(19) United States
(12) Patent Application Publication
(10) Pub. No.: US 2024/0285795 A1
(43) Pub. Date: Aug. 29, 2024

(54) TARGETS FOR RNA THERAPEUTICS

(71) Applicant: CureVac SE, Tübingen (DE)

(72) Inventor: Ingmar HOERR, Tübingen (DE)

(73) Assignee: CureVac SE, Tübingen (DE)

(21) Appl. No.: 17/199,120

(22) Filed: Mar. 11, 2021

Related U.S. Application Data
(62) Division of application No. 15/585,561, filed on May 3, 2017, now abandoned.

(30) Foreign Application Priority Data
May 4, 2016 (EP) 16001039.3

(51) Int. Cl. A61K 48/00; A61K 9/00; A61K 39/00; C07K 14/00

(52) U.S. Cl. CPC A61K 48/00; A61K 9/0014; A61K 9/0019; A61K 48/005; C07K 14/00; A61K 39/00; A61K 48/0075

(57) ABSTRACT

The present invention relates to a method for treating or preventing a disease, disorder or condition by administration of a polynucleotide, e.g. a modified RNA, encoding a peptide or protein related to this disease, disorder or condition. The present invention also relates to pharmaceutical compositions for use in such method.

This application is a divisional of U.S. application Ser. No. 15/585,561, filed May 3, 2017, which claims priority to European Application No. 16001039.3, filed May 4, 2016, the entire contents of each of which are hereby incorporated by reference.

### INCORPORATION OF SEQUENCE LISTING

The instant application contains a Sequence Listing, which has been submitted in ASCII format in U.S. Application Ser. No. 15/585,561, filed May 3, 2017 and is hereby incorporated by reference in its entirety. Said ASCII copy, submitted on Jan. 8, 2018 on compact discs by mail, is named CRVCO220US_ST25.txt and is 895 MB (as measured in Microsoft Windows®). The file was separated into three concatenated files for copy to two CD-ROMs.

### FIELD OF THE INVENTION

The present invention relates to a method for treating or preventing a disease, disorder or condition by administration of a polynucleotide, e.g. a modified RNA, encoding a peptide or protein related to this disease, disorder or condition. The present invention also relates to pharmaceutical compositions for use in such method.

### BACKGROUND OF THE INVENTION

Gene therapy and genetic vaccination belong to the most promising and quickly developing methods of modern medicine. Both methods are based on the introduction of nucleic acids into cells or into tissues of the patient and on subsequent processing of the information encoded by the nucleic acids introduced, i.e. expression of the desired protein or peptide.

While DNA is known to be relatively stable and easy to handle, the use of DNA in therapy bears the risk of undesired insertion of the administered DNA fragments into the patient's genome potentially resulting in loss of function of the genes. As a further risk, the undesired generation of anti-DNA antibodies has emerged. Another drawback is the limited expression level of the encoded peptide or protein that can be achieved by DNA administration and its subsequent transcription/translation.

By using RNA molecules instead of DNA molecules for gene therapy or genetic vaccination, the risk of undesired genomic integration and generation of anti-DNA antibodies is minimized or is even prevented completely. For many years it was generally accepted that mRNA is too unstable to be efficiently used for gene therapy purposes. In the last decade, however, several research groups faced this challenge and not only proved the feasibility of mRNA-mediated transfection with surprising results regarding transfection efficiency and duration of protein expression, but were also able to demonstrate major advantages over the use of DNA. One of these advantages is that mRNA does not need to cross the nuclear barrier for its encoded proteins to be expressed (reviewed in Tavernier et al., J Control Release. (2011): 150(3):238-47.

Although progress has been made in this field, there is a continuing need for methods of specifically treating diseases, disorders or conditions by using a polynucleotide encoding a specific protein or peptide as medicament.

### SUMMARY OF THE INVENTION

The present invention relates to a method for treating or preventing a disease, disorder or condition in a subject in need thereof by increasing the level of at least one peptide or protein related to this disease, disorder or condition comprising administering to the subject a pharmaceutical composition comprising at least one polynucleotide encoding said at least one peptide or protein.

wherein said at least one peptide or protein is selected from the group consisting of peptides and proteins listed in column 1 (c1) of Table 1 and wherein the disease, disorder or condition to which said at least one peptide or protein relates is selected from the group consisting of diseases, disorders and conditions listed in column 7 (c7) of Table 1.

wherein the at least one peptide or protein and the disease, disorder or condition to which said at least one peptide or protein relates are listed in Table 1 in the following order: c1(Peptide or protein or gene) c2(NCBI RefSeq ID) c3(NCBI RefSeq description) c4(Protein SEQ ID NO) c5(DNA SEQ ID NOs) c6(RNA SEQ ID NOs) c7(Related disease, disorder or condition); proteins are separated by #.

The present invention further relates to a method for reducing and/or ameliorating at least one symptom of a disease, disorder or condition in a subject in need thereof by increasing the level of at least one peptide or protein related to this disease, disorder or condition comprising administering to the subject a pharmaceutical composition comprising a polynucleotide encoding said at least one peptide or protein,

wherein said at least one peptide or protein is selected from the group consisting of peptides and proteins listed in column 1 (c1) of Table 1 and wherein the disease, disorder or condition to which said peptide or protein relates is selected from the group consisting of diseases, disorders and conditions listed in column 7 (c7) of Table 1.

wherein the at least one peptide or protein and the disease, disorder or condition to which said at least one peptide or protein relates are listed in Table 1 in the following order: c1(Peptide or protein or gene) c2(NCBI RefSeq ID) c3(NCBI RefSeq description) c4(Protein SEQ ID NO) c5(DNA SEQ ID NOs) c6(RNA SEQ ID NOs) c7(Related disease, disorder or condition); proteins are separated by #. As Table 1 is depicted as running text the term “a row” is used herein for all information associated to one peptide or protein. Consequently, an entry beginning with a number sign and ending with a semicolon resembles a “row” presenting a specific peptide or protein or gene and also its specific features like f.e. NCBI RefSeq ID, NCBI RefSeq description, Protein SEQ ID NO, DNA SEQ ID NOS, RNA SEQ ID NOs. and related disease(s), disorder(s) or condition(s).

In other words, all information comprised in column 1 (c1) (Peptide or protein or gene), column 2 (c2)(NCBI RefSeq ID), column 3 (c3) (NCBI RefSeq description), column 4 (c4) (Protein SEQ ID NO), column 5 (c5) (DNA SEQ ID NOs), column 6 (c6) (RNA SEQ ID NOs) and column 7 (c7) (Related disease, disorder or condition) relating to one peptide or protein (as named in column 1 (c1)) corresponds to the information which would have been disclosed in one row if depicted as table. The term “column (“accordingly equals the term “c1”. “column 2” equals the term “c2”. “column 3” equals the term “c3”. “column 4” equals the term “c4”. “column 5” equals the term “c5”. “column 6” equals the term “c6”, and “column 7” equals the term “c7” and vice versa. As Table 1 is depicted as a running text, each peptide or protein or gene entry (i.e. peptide or protein or gene of the invention) is separated by a semicolon and, for ease of readability, each entry is preluded by the number sign (#).

Table 1accordingly starts with entry “c1(A1BG) c2(NP_570602) c3(alpha-1B-glycoprotein precursor) c4(234076) c5(234077, 234078, 234079, 234080, 234081, 234082) c6( ) c7(b, d, c, e, h, g, i, f)” and ends with entry “c1(ZZZ3) c2(XP_011539507) c3(ZZ-type zinc finger-containing protein 3 isoform X3) c4(337706) c5(337707, 337708, 337709, 337710, 337711, 337712, 337713) c6( ) c7(v)” (all in accordance with the formula and the Abbreviation Dictionary as described above; number signs (#) and semicolons were introduced for ease of readability to separate the different entries from another).

According to this, exemplarily the third entry of a peptide or protein gene as disclosed in Table 1 i.e. “#c1(A1CF) c2(NP_001185749) c3(APOBEC1 complementation factor isoform 3) c4(76200, 76201) c5(76202, 76203, 76204, 76205, 76206, 76207, 76208, 76209, 76210, 76211, 76212, 76213) c6( ) c7(x, y, l, A, c, s, u, w, z, r, h, n, v, j, m, k, g, t, q, o, p)” resembles the third entry (peptide or protein or gene) shown in Table A1.

TABLE A1
Exemplary depiction from the third entry as disclosed in Table 1 (c1, c2, c3, c4, c5, c6 and c7 correspond to the
corresponding entry under column 1, column 2, column 3, column 4, column 5, column 6 and column 7) in accordance with formula
column 1 (cl) (Peptide or protein or gene), column 2 (c2)(NCBI RefSeq ID), column 3 (c3) (NCBI RefSeq description), column 4 (c4)
(Protein SEQ ID NO), column 5 (c5) (DNA SER ID NOs), column 6 (c6) (RNA SEQ ID NOs) and column 7 (c7) (Related disease, disorder
or condition) and the Abbreviation Dictionary as disclosed in Table C - emphasis added for corresponding entry data (italics and
underlined). Each of the recited sequences are also provided in the sequence listing of the priority document European Application No.
16001039.3, filed May 4, 2016, the entire contents of which is hereby incorporated by reference.

| column 1 | | | | | | column 7 |
|---|---|---|---|---|---|---|
| (i.e. feature cl) | column 2 | column 3 | column 4 | column 5 | column 6 | (i.e. feature 7) |
| Peptide or | (i.e. feature c2) | (i.e. feature c3) | (i.e. feature c4) | (i.e. feature c5) | (i.e. feature c6) | Related disease, |
| protein or | NCBI | NCBI RefSeq | Protein SEQ ID | DNA SEQ ID | RNA SEQ ID | disorder or |
| gene | RefSeq ID | description | NO | NOs | NOs | condition |
| AICF | NP 001185749 | APOBECI | 76200, 76201 | 76202, 76203, | | coronary artery |
| | | complementation | | 76204, 76205, | | disease; precursor |
| | | factor isoform | | 76206, 76207, | | cell lymphoblastic |
| | | 3 | | 76208, 76209, | | leukemia-lymphoma; |
| | | | | 76210, 76211, | | cholelithiasis; ovarian |
| | | | | 76212, 76213 | | carcinoma; recurrent |
| | | | | | | malignant neoplasm; |
| | | | | | | juvenile rheumatoid |
| | | | | | | arthritis; |
| | | | | | | mitochondrial |
| | | | | | | cardiomyopathy; large |
| | | | | | | intestine cancer; |
| | | | | | | amyotrophyic lateral |
| | | | | | | sclerosis; colorectal |
| | | | | | | cancer; adenoma; |
| | | | | | | ovarian cancer; |
| | | | | | | cholecystolithiasis; |
| | | | | | | obesity; fibrodysplasia |
| | | | | | | ossificans |
| | | | | | | progressiva; |
| | | | | | | adenocarcinoma; |
| | | | | | | human |
| | | | | | | immunodeficiency |
| | | | | | | virus infectious |
| | | | | | | disease; acute |
| | | | | | | lymphocytic leukemia; |
| | | | | | | rheumatoid arthritis; |
| | | | | | | allergic asthma; type I |
| | | | | | | diabetes mellitus |

Moreover, the invention relates to a pharmaceutical composition comprising a polynucleotide encoding at least one peptide or protein for use in treating or preventing a disease, disorder or condition in a subject, wherein the at least one peptide or protein is related to said disease, disorder or condition and is selected from the group consisting of peptides and proteins listed in column 1 (c1) of Table 1 and wherein the disease, disorder or condition to which said at least one peptide or protein relates is selected from the group consisting of diseases, disorders and conditions listed in column 7 (c7) of Table 1, wherein the disease-, disorder- or condition-related peptide or protein and the disease, disorder or condition to which the at least one peptide or protein relates are listed in Table 1 in the following order: c1(Peptide or protein or gene) c2(NCBI RefSeq ID) c3(NCBI RefSeq description) c4(Protein SEQ ID NO) c5(DNA SEQ ID NOs) c6(RNA SEQ ID NOs) c7(Related disease, disorder or condition); proteins are separated by #. In a further aspect, the invention relates to a pharmaceutical composition comprising a polynucleotide encoding at least one peptide or protein for use in reducing and/or ameliorating at least one symptom of a disease, disorder or condition a disease, disorder or condition in a subject, wherein the at least one peptide or protein is related to said disease and is selected from the group consisting of peptides and proteins listed in column 1 (c1) of Table 1 and wherein the disease, disorder or condition to which said at least one peptide or protein relates is selected from the group consisting of diseases, disorders and conditions listed in column 7 (c7) of Table 1, wherein the at least one peptide or protein and the disease, disorder or condition to which the peptide or protein relates are listed in Table 1 in the following order: c1(Peptide or protein or gene) c2(NCBI RefSeq ID) c3(NCBI RefSeq description) c4(Protein SEQ ID NO) c5(DNA SEQ ID NOs) c6(RNA SEQ ID NOs) c7(Related disease, disorder or condition); proteins are separated by #.

In one aspect, the invention relates to a pharmaceutical composition comprising a polynucleotide encoding a disease-, disorder- or condition-related peptide or protein, wherein the disease-, disorder- or condition-related peptide or protein is selected from the group consisting of peptides and proteins listed in column 1 (c1) of Table 1.

In a further aspect, the invention relates to a polynucleotide encoding a disease-, disorder- or condition-related peptide or protein, wherein the disease-, disorder- or condition-related peptide or protein is selected from the group consisting of peptides and proteins listed in column 1 (c1) of Table 1.

The disease-, disorder- or condition-related peptide or protein may have an amino acid sequence of any of SEQ ID NOs listed in column 4 (c4) of Table 1, and/or may be encoded by a nucleic acid sequence of any of SEQ ID NOs listed in column 5 (c5) and column 6 (c6) of Table 1, wherein the disease-, disorder- or condition-related peptide or protein and its corresponding SEQ ID NO are listed in Table 1 in the following order: c1(Peptide or protein or gene) c2(NCBI RefSeq 10) c3(NCBI RefSeq description) c4(Protein SEQ ID NO) c5(DNA SEQ ID NOs) c6(RNA SEQ ID NOs) c7(Related disease, disorder or condition); proteins are separated by #.

The pharmaceutical composition may comprise a lipid which is selected from the group consisting of DLin-DMA, DLin-K-DMA, DLin-KC2-DMA, 98N12-5, C12-200, DLin-MC3-DMA, PLGA, PEG, PEG-DMG and PEGylated lipids and mixtures thereof.

The pharmaceutical composition may further comprise a pharmaceutically acceptable excipient.

The pharmaceutically acceptable excipient may be selected from the group consisting of a solvent, aqueous solvent, nonaqueous solvent, dispersion media, diluent, dispersion, suspension aid, surface active agent, isotonic agent, thickening or emulsifying agent, preservative, lipid, lipidoid, liposome, lipid nanoparticle, core-shell nanoparticles, polymer, lipoplex, peptide, protein, cell, hyaluronidase, and mixtures thereof.

The pharmaceutical composition may be administered by injection, topical administration, intranasal administration or ophthalmic administration. The injection may include intradermal injection, subcutaneous injection and intramuscular injection. The pharmaceutical composition administered by topical administration may further comprise a penetration enhancer. This penetration enhancer may be selected from the group consisting of surfactants, fatty acids, bile salts, chelating agents, non-chelating non-surfactants, polyoxyethylene-9-lauryl ether, polyoxyethylene-20-cetyl ether, fatty acids and/or salts in combination with bile acids and/or salts, sodium salt in combination with lauric acid, capric acid and UDCA, and the like.

The pharmaceutical composition may be administered at a total daily dose of between 0.001 μg and 150 ug. Further, the pharmaceutical composition may be administered two or more times.

The polynucleotide according to the present invention may comprise at least one modification.

The at least one modification may be a modification selected from the group consisting of codon optimization, UTR modification, addition of a poly(A) tail, addition of a 5′-CAP structure, and incorporation of at least one chemically modified nucleotide.

The 5′-CAP structure may be selected from the group consisting of Cap1. ARCA, inosine, Ni-methyl-guanosine, 2′-fluoro-guanosine, 7-deaza-guanosine, 8-oxo-guanosine, 2-amino-guanosine, LNA-guanosine, and 2-azido-guanosine. The at least one modification may be the addition of a chemically modified ARCA-CAP. The ARCA-CAP may be modified with phosphorothioate.

The chemically modified nucleotide may be selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, I-methyl-pseudouridine, 4-thio-1-methyl-pseudouridine, 2-thio-1-methyl-pseudouridine, 1-methyl-1-deaza-pseudouridine, 2-thio-1-methyl-I-deaza-pseudouridine, dihydrouridine, dihydropseudouridine, 2-thia-dihydrouridine, 2-thia-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonylcarbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In one embodiment, the polynucleotide comprises at least two modifications.

In a further embodiment, the polynucleotide is substantially purified.

### DETAILED DESCRIPTION OF THE INVENTION

The present invention as illustratively described in the following may suitably be practiced in the absence of any element or elements, limitation or limitations, not specifically disclosed herein.

The present invention will be described with respect to particular aspects and embodiments, but the invention is not limited thereto. Terms as set forth hereinafter are generally to be understood in their common sense unless indicated otherwise.

Where the term “comprising” is used in the present description and claims, it does not exclude other elements. For the purposes of the present invention, the term “consisting of” is considered to be a preferred embodiment of the term “comprising of”. If hereinafter e.g. a group is defined to comprise at least a certain number of elements, this is to be understood to always also disclose a group which preferably consists only of these elements. If an aspect or embodiment of the invention is defined to comprise at least certain compositional, structural and/or functional features, this is to be understood to always also disclose an aspect and embodiment, which preferably consists only of these features.

Where an indefinite or definite article is used when referring to a singular noun, e.g. “a”. “an” or “the”, this includes a plural of that noun unless something else is specifically stated.

All documents (including all patents, patent applications, scientific publications, manufacturer's specifications, instructions, etc.) specifically mentioned herein are incorporated by reference in their entirety for all purposes including describing and disclosing the chemicals, instruments, and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The present inventors have related more than 29 000 human proteins and peptides to specific diseases, disorders or conditions (see Table 1). Thus, the present invention provides pharmaceutical compositions comprising a polynucleotide or a plurality of polynucleotides encoding a specific disease-, disorder- and condition-related peptide or protein that provide highly specific and individual treatments of a large variety of diseases, disorders and conditions, e.g. in the field of gene therapy or genetic vaccination. In addition, administering to a subject pharmaceutical compositions comprising polynucleotides encoding such specific disease -, disorder- and condition-related peptides and proteins may prevent (early) onset of such specific diseases, disorders and conditions. Further, such compositions may be used for reducing and/or ameliorating at least one symptom of such specific diseases, disorders or conditions.

The peptides and proteins and the diseases, disorders and/or conditions related thereto are shown in Table 1 below wherein each row of the Table provides information for one specific protein, i.e. information on its function (column 3 (c3), the amino acid sequence (column 4 (c4)), the DNA sequence encoding the protein (column 5 (c5) and the sequence of the corresponding RNA (column 6 (c6). Finally and importantly, column 7 (c7) lists diseases, disorders and/or conditions related to the protein of column 1 (c1).

Based on this information, it is within the ordinary skills of those in the art to select a protein or peptide which relates to a particular disease, disorder or condition and therefore can be used to treat this disease, disorder or condition and to create a polynucleotide, preferably an RNA, encoding this protein or peptide based on the sequence information in columns 5 (c5) and 6 (c6) of Table 1. If several different proteins or peptides are related to the same disease, disorder or condition, a mixture of different polynucleotides can be created, wherein each type of polynucleotide encodes one of said proteins or peptides related to the specific disease, disorder or condition.

For instance, according to entry 1 of Table 1, i.e. “#c1(A1BG) c2(NP_570602) c3(alpha-1B-glycoprotein precursor) c4(234076) c5(234077, 234078, 234079, 234080, 234081, 234082) c6( ) c7(b, d, c, e, h, g, i, f)”, the alpha-1B-glycoprotein precursor (A1BG) (see columns 1 (c1) and 3 (c3) of Table 1) is related to the following abbreviated diseases, disorders or conditions which are indicated in column 7 (c7) of Table 1): “b, d, c, e, h, g, i, f”. The abbreviations “b, d, c, e, h, g, i, f” as disclosed under feature c7 are to be read from the Abbreviation Dictionary for the “Related disease, disorder or condition” as shown in Table C, whereby each abbreviation is depicted with a specific “Related disease, disorder or condition” in written form, f.e, prostate cancer corresponds to “A”. Accordingly. Table C disclosed the following “Related diseases, disorders or conditions” for entry 1 i.e. alpha-1B-glycoprotein precursor: spots on skin, amyotrophyic lateral sclerosis, glioblastoma multiforme, cardiovascular system disease, down syndrome, adenocarcinoma, human immunodeficiency virus infectious disease, and exanthema. The SEQ ID Nos of the amino acid sequences of the disease-, disorder- or condition-related peptide or protein are listed in column 4 (c4) of Table 1, while columns 5 (c5) and 6 (c6) show the corresponding DNA and RNA sequences, respectively. Additionally, the NCBI RefSeq IDs of the disease-, disorder- or condition-related peptide or protein and information on the function of the protein are shown in columns 2 (c2) and 3 (c3) of Table 1, respectively.

Likewise, the following entries 2 to 29710 of Table 1 (as described above, each entry is preluded by the number sign # and ends with a semicolon) represent the other peptides and proteins that were found and the related specific diseases, disorders and conditions. Accordingly, if one wishes to match a specific peptide or protein with a disease, disorder or condition, he may find this peptide or protein in column 1 (cf) of Table 1 and the related disease, disorder or condition in column 7 (c7) in the same entry of Table 1. The nucleic acid sequences needed to construct the polynucleotide therapeutic can be identified in columns 5 (c5) and/or 6 (c6) of that entry in Table 1.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered as disclosed by the description of the present application, unless the context indicates otherwise.

Where nucleic acid sequences are reported in the context of the present invention, these sequences generally comprise both, the specific RNA or DNA sequence as well as its corresponding DNA or RNA counterpart, respectively. For example, where a DNA sequence is provided, the skilled person knows that the corresponding RNA sequence is obtained by exchange of thymine by uracil residues and vice versa.

Generally, the nomenclature used herein and the laboratory procedures are those well known and commonly employed in the art. Standard techniques are used for nucleic acid synthesis. The techniques and procedures are generally performed according to conventional methods in the art and various general references (e.g., Sambrook et al., 1989, Molecular Cloning: A Laboratory Manual, 2d ed. Cold Spring Harbor Laboratory Press, Cold Spring Harbor, NY), which are provided throughout this document.

Gene therapy: Gene therapy may typically be understood to mean a treatment of a patient's body or isolated elements of a patient's body, for example isolated tissues/cells, by nucleic acids encoding a peptide or protein. It typically may comprise at least one of the steps of a) administration of a nucleic acid, preferably a polynucleotide molecule as defined herein, directly to the patient—by whatever administration route—or in vitro to isolated cells/tissues of the patient, which results in transfection of the patient's cells either in vivo/ex vivo or in vitro; b) transcription and/or translation of the introduced nucleic acid molecule; and optionally c) re-administration of isolated, transfected cells to the patient, if the nucleic acid has not been administered directly to the patient.

Genetic vaccination: Genetic vaccination may typically be understood to be vaccination by administration of a nucleic acid molecule encoding an antigen or an immunogen or fragments thereof. The nucleic acid molecule may be administered to a subject's body or to isolated cells of a subject. Upon transfection of certain cells of the body or upon transfection of the isolated cells, the antigen or immunogen may be expressed by those cells and subsequently presented to the immune system, eliciting an adaptive, i.e. antigen-specific immune response. Accordingly, genetic vaccination typically comprises at least one of the steps of a) administration of a nucleic acid, preferably a polynucleotide molecule as defined herein, to a subject, preferably a patient, or to isolated cells of a subject, preferably a patient, which usually results in transfection of the subject's cells either in vivo or in vitro, b) transcription and/or translation of the introduced nucleic acid molecule; and optionally c) re-administration of isolated, transfected cells to the subject, preferably the patient, if the nucleic acid has not been administered directly to the patient.

According to the invention, the polynucleotide is designed to encode one or more disease-, disorder- or condition-related peptides or proteins as listed in column 1 (c1) of Table 1 and is used to treat or prevent a related disease, disorder or condition as listed in column 7 (c7) of the same entry in Table 1. Further, such polynucleotide can be used for reducing and/or ameliorating a symptom of a disease, disorder or condition.

According to the invention, the term “protein” refers to a polypeptide having at least 20 amino acids. A “peptide” is understood to contain typically less than 20 amino acids. Fragments, functional protein domains and variants are also considered to be within the scope of peptides or proteins of this invention.

The term “fragment” as used herein refers to a peptide or protein which has the identical amino acid sequence, i.e. the identical succession of amino acid residues, as the reference sequence, but the amino acid sequence is at least one amino acid residue shorter than a native or reference sequence. Generally, fragments of a peptide or protein will have at least about 10%, 20%, 30%, 40%, 45%, 50%, 55%, 60%, 65%, 70%, 75%, 80%, 85%, 90%, 91%, 92%, 93%, 94%, 95%, 96%, 97%, 98%, 99% but less than 100% of the length of the disease-, disorder- or condition-related peptide or protein as listed in column 1 (c1) of Table 1 or the amino acid sequence of the peptide or protein as shown in column 4 (c4) of Table 1.

The term “variant” as used herein refers to proteins or peptides which differ in their amino acid sequence from a native or reference sequence. The variants may possess substitutions, deletions, and/or insertions at certain positions within the amino acid sequence, as compared to a native or reference sequence. Generally, variants of a disease-, disorder- or condition-related peptide or protein as listed in column 1 (c1) of Table 1 will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity (homology) to the reference sequence, i.e. the amino acid sequence of the peptide or protein as shown in column 4 (c4) of Table 1. Methods and computer programs for calculating sequence identity and homology, respectively, are well known in the art.

The term “polynucleotide” as used herein refers to a polymer of nucleotides. Examples for polynucleotides according to the invention are ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) and hybrids and combinations thereof. Preferably, the polynucleotide to be used in the present invention is RNA.

RNA: RNA is the usual abbreviation for ribonucleic acid. It is a nucleic acid molecule, i.e. a polymer consisting of nucleotide monomers. These nucleotides are usually adenosine-monophosphate (AMP), uridine-monophosphate (UMP), guanosine-monophosphate (GMP) and cytidine-monophosphate (CMP) monomers or analogs thereof, which are connected to each other along a so-called backbone. The backbone is formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA sequence. Usually RNA may be obtainable by transcription of a DNA sequence, e.g., inside a cell. In eukaryotic cells, transcription is typically performed inside the nucleus or the mitochondria. In vivo, transcription of DNA usually results in the so-called premature RNA which has to be processed into so-called messenger-RNA, usually abbreviated as mRNA. Processing of the premature RNA, e.g. in eukaryotic organisms, comprises a variety of different post-transcriptional modifications such as splicing. 5′-capping, polyadenylation, export from the nucleus or the mitochondria and the like. The sum of these processes is also called maturation of RNA. The mature messenger RNA usually provides the nucleotide sequence that may be translated into an amino acid sequence of a particular peptide or protein. Typically, a mature mRNA comprises a 5′-cap, optionally a 5′UTR, a coding sequence, optionally a 3′UTR and a poly(A) sequence.

Suitable RNA sequences for use in the present invention are shown in column 6 (c6) of Table 1. Further, it is possible to create a suitable RNA sequence based on the sequence of the corresponding DNA which is shown in column 5 (c5) of Table 1.

Fragments and variants of the RNA sequences are also considered to be within the scope of RNA sequences of this invention.

The term “fragment” as used herein refers to an RNA which has the identical nucleic acid sequence, i.e. the identical succession of nucleotides, as the reference sequence, but the nucleic acid sequence is at least one nucleotide shorter than a native or reference sequence. Generally, fragments of an RNA will have at least about 10%, 20%, 30%, 40%, 45%, 50%, 55%, 60%, 65%, 70%, 75%, 80%, 85%, 90%, 91%, 92%, 93%, 94%, 95%, 96%, 97%, 98%, 99% but less than 100% of the length of the RNA sequence listed in column 6 (c6) of Table 1 or of the corresponding DNA sequence listed in column 5 (c5) of Table 1.

The term “variant” as used herein refers to RNAs which differ in their nucleic acid sequence from a native or reference sequence. The variants may possess substitutions, deletions, and/or insertions at certain positions within the nucleic acid sequence, as compared to a native or reference sequence. Generally, variants of an RNA sequence as listed in column 6 (c6) of Table 1 or of the corresponding DNA sequence listed in column 5 (c5) of Table 1 will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity (homology) to the reference sequence. i.e. the RNA sequence as listed in column 6 (c6) of Table 1 or of the corresponding DNA sequence listed in column 5 (c5) of Table 1. Methods and computer programs for calculating sequence identity and homology, respectively, are well known in the art.

Bicistronic RNA, multicistronic RNA: A bicistronic or multicistronic RNA is typically an RNA, that typically may have two (bicistronic) or more (multicistronic) open reading frames (ORF). An open reading frame in this context is a sequence of codons that is translatable into a peptide or protein.

Pharmaceutical composition: In one aspect, the present invention relates to the use of a polynucleotide encoding a disease-, disorder- or condition-related peptide or protein as defined herein for the preparation of a pharmaceutical composition. In a particular preferred embodiment, a pharmaceutical composition according to the invention comprises a plurality of polynucleotides as defined herein. As discussed above, the pharmaceutical composition may also comprise two or more different polynucleotides encoding two or more different peptides or proteins wherein the different peptides or proteins relate to the same disease, disorder or condition.

Pharmaceutical compositions according to the invention comprise a polynucleotide as described herein and additionally may comprise other components. Components suitable for the manufacture of pharmaceutical compositions comprising a polynucleotide as described herein are known in the art and are e.g. described in WO 2013/052523 A1, WO 2013/090648, WO 2013/142958 A1, WO 2013/15/736 A1, WO 2014/081507 A1, WO 2014/093924 A1, WO 2014/164253 A1, WO 2013/151663 A1, WO 2013/151664 A1, WO 2013/151665 A2, WO 2013/151666 A1, WO 2013/151667 A1, WO 2013/151668 A2, WO 2013/151669 A1, WO 2013/151670 A2. WO 2013/151671 A1, and WO 2013/151672 A1.

In a preferred aspect, the present invention provides a pharmaceutical composition comprising a polynucleotide as defined herein, or a pharmaceutical composition comprising a plurality of polynucleotides as defined herein and optionally a pharmaceutically acceptable lipid. In a particular preferred embodiment the lipid is selected from the group consisting of DLin-DMA, DLin-K-DMA, DLin-KC2-DMA, 98N12-5, C12-200. DLin-MC3-DMA, PLGA poly(lactic acid-co-glycolic acid, PEG (polyethyleneglycol). PEG-DMG (1,2-dimyristoyl-sn-glycero-3 -phosphoethanol-amine-N-methoxy(polyethyleneglycol) and PEGylated lipids and mixtures thereof. Synthesis and structures of DLin-DMA, DLin-K-DMA, DLin-KC2-DMA, 98N12-5. C12-200 and DLin-MC3-DMA are described in WO 2013/151672 A2.

In a further preferred embodiment, the present invention also provides a pharmaceutical composition, comprising a polynucleotide as defined herein or a composition comprising a plurality of polynucleotides as defined herein and a pharmaceutically acceptable carrier for the polynucleotide.

Carrier/polymeric carrier: A carrier in the context of the invention may typically be a compound that facilitates transport and/or complexation of another compound (cargo). A polymeric carrier is typically a carrier that is formed of a polymer. A carrier may be associated to its cargo by covalent or non-covalent interaction. A carrier may transport nucleic acids, e.g. a polynucleotide or DNA, to the target cells.

As a first ingredient, the pharmaceutical composition comprises at least one polynucleotide as defined herein.

As a second ingredient the pharmaceutical composition may optionally comprise at least one additional pharmaceutically active component. A pharmaceutically active component in this connection is a compound that has a therapeutic effect to treat, ameliorate or prevent a particular indication or disease as mentioned herein. Such compounds include, without implying any limitation, peptides or proteins, preferably as defined herein, nucleic acids, preferably as defined herein, (therapeutically active) low molecular weight organic or inorganic compounds (molecular weight less than 5000, preferably less than 1000), sugars, antigens or antibodies, preferably as defined herein, therapeutic agents already known in the prior art, antigenic cells, antigenic cellular fragments, cellular fractions; cell wall components (e.g. polysaccharides), attenuated or de-activated (e.g. chemically or by irradiation) pathogens (virus, bacteria etc.), adjuvants, preferably as defined herein, etc.

Furthermore, the pharmaceutical composition may comprise a pharmaceutically acceptable excipient. In the context of the present invention, a pharmaceutically acceptable excipient includes a solvent, aqueous solvent, nonaqueous solvent, dispersion media, diluent, dispersion, suspension aid, surface active agent, isotonic agent, thickening or emulsifying agent, preservative, lipid, lipidoid, liposome, lipid nanoparticle, core-shell nanoparticles, polymer, lipoplex, peptide, protein, cell, hyaluronidase, etc.

The pharmaceutical composition according to the present invention may be administered to a subject in need thereof, for example, a patient, by any suitable administration route, such as by oral, topical, rectal, vaginal, dermal, intra-tumoural, nasal, lingual, ophthalmic, parenteral administration or administration by inhalation, insufflation, injection, infusion or by enema. Thus, pharmaceutical composition according to the present invention may be adapted, for example, for oral, topical, rectal, vaginal, dermal, intra-tumoural, nasal, lingual, ophthalmic, parenteral administration or administration by inhalation, insufflation, injection, infusion or by enema. Furthermore, the method of treatment according to the present invention may comprise oral, topical, rectal, vaginal, dermal, intra-tumoural, nasal, lingual, parenteral administration or administration by inhalation, insufflation, injection, infusion or by enema. Preferred administration routes are oral and parenteral administration, such as intravenous, intramuscular, subcutaneous, intranodal, intralymphatic, intra-tumoural or intraperitoneal injection or transdermal delivery.

The polynucleotide of the present invention may be administered at a total daily dose of between 0.001 μg and 150 ug.

The pharmaceutical composition for topical administration may be a cream, lotion, ointment, gel, spray, solution and the like. The pharmaceutical composition for topical administration may further include a penetration enhancer such as surfactants, fatty acids, bile salts, chelating agents, non-chelating non-surfactants, polyoxyethylene-9-lauryl ether, polyoxyethylene-20-cetyl ether, fatty acids and/or salts in combination with bile acids and/or salts, sodium salt in combination with lauric acid, capric acid and UDCA, and the like.

In the context of the present invention, a total daily dose is an amount given or prescribed in a 24 hour period. It may be administered as a single unit dose. In one embodiment, the polynucleotides used in the present invention are administered to a subject in split doses. In the context of the present invention, a split dose is the division of single unit dose or total daily dose into two or more doses, e.g. two or more administrations of the single unit dose. In the context of the present invention, a single unit dose is a dose of any therapeutic administered in one dose/at one time/ single route/ single point of contact, i.e., in a single administration event.

The term subject as used herein refers to any organism to which a composition in accordance with the invention may be administered. Typical subjects include animals, preferably mammals such as mice, rats, rabbits, non-human primates, and humans. Preferably, the subject is a human.

The term “symptom” as used herein is a signal or a sign of a disease, disorder or condition. Examples of symptoms include, but are not limited to, weakness, aches and pains, fever, fatigue, weight loss, blood clots, increased blood calcium levels, etc.

The term “treating” as used herein refers to partially or completely alleviating, ameliorating, improving, relieving, inhibiting progression of, and/or reducing severity of a disease, disorder, or condition.

The term “preventing” as used herein refers to partially or completely delaying onset of and/or reducing incidence of a disease, disorder, or condition

The term “purified” as used herein is understood to mean that the desired polynucleotide in a sample is separated and/or isolated from impurities, intermediates, byproducts and/or reaction components present therein or that the impurities, intermediates, byproducts and/or reaction components are at least depleted from the sample comprising the polynucleotide. Non-limiting examples of undesired constituents of polynucleotide-containing samples which therefore need to be depleted may comprise degraded fragments or fragments which have arisen as a result of premature termination of transcription, or also excessively long transcripts if plasmids are not completely linearized. Furthermore, intermediates may be depleted from the sample such as e.g. template DNA. Additionally, reaction components such as enzymes, proteins, bacterial DNA and RNA, small molecules such as spermidine, buffer components etc. may have to be depleted from the polynucleotide sample. In addition, impurities such as organic solvents and nucleotides or other small molecules may be separated. The purity may be determined by methods commonly known to the skilled person, e.g. by gas chromatography, quantitative PCR, analytical HPLC or gel electrophoresis. By “substantially purified” is meant that the polynucleotide is substantially separated from the environment in which it was formed. Substantial purification may include samples containing at least about 50%, at least about 60%, at least about 70%, at least about 80%, at least about 90%, at least about 95%, at least about 97%, or at least about 99% by weight of the polynucleotide of the present invention or salt thereof.

In a particularly preferred aspect of the present invention, the polynucleotide as defined herein is a modified polynucleotide comprising at least one modification. In an even more preferred embodiment, the modified polynucleotide is a modified RNA and in a most preferred embodiment the modified polynucleotide is chemically modified RNA.

Suitable modifications of polynucleotide are known in the art and e.g. described in WO 2013/052523 A1, WO 2013/090648, WO 2013/142958 A1, WO 2013/15/736 A1, WO2014/081507 A1, WO 2014/093924 A1, WO 2014/164253 A1, WO 2013/151663 A1, WO 2013/151664 A1. WO 2013/151665 A2, WO 2013/151666 A1, WO 2013/151667 A1, WO 2013/151668 A2, WO 2013/151669 A1, WO 2013/151670 A2, WO 2013/151671 A1, and WO 2013/151672 A1.

Chemical modifications: The term “chemical modification” as used herein refer to modification with respect to adenosine (A), guanosine (G), uridine (U), thymidine (T) or cytidine (C) ribo- or deoxyribonucleotides in one or more of their position, pattern, percent or population. Generally, herein, these terms are not intended to refer to the ribonucleotide modifications in naturally occurring 5′-terminal mRNA cap moieties.

The modifications may be various distinct modifications. In some embodiments, the polynucleotides may contain one, two, or more (optionally different) nucleoside or nucleotide modifications. In some embodiments, a modified polynucleotide, introduced to a cell may exhibit reduced degradation in the cell, as compared to an unmodified polynucleotide.

Modifications which are useful in the present invention include, but are not limited to, those in Table A. Noted in the table are the symbol of the modification, the nucleobase type and whether the modification is naturally occurring or not.

TABLE A
Modifications

| Name | Symbol | Base | Naturally occouring |
|---|---|---|---|
| 2-methylthio-N6-(cishydroxyisopentenyl)adenosine | ms2i6A | A | Yes |
| 2-methylthio-N6-methyladenosine | ms2m6A | A | Yes |
| 2-methylthio-N6-threonyl-carbamoyladenosine | ms2t6A | A | Yes |
| N6-glycinylcarbamoyladenosine | g6A | A | Yes |
| N6-isopentenyladenosine | i6A | A | Yes |
| N6-methyladenosine | m6A | A | Yes |
| N6-threonylcarbamoyladenosine | t6A | A | Yes |
| 1,2′-O-dimethyladenosine | mlAm | A | Yes |
| 1-methyladenosine | mlA | A | Yes |
| 2′-O-methyladenosine | Am | A | Yes |
| 2′-O-ribosyladenosine(phosphate) | Ar(p) | A | Yes |
| 2-methyladenosine | m2A | A | Yes |
| 2-methylthio-N6 isopentenyladenosine | ms2i6A | A | Yes |
| 2-methylthio-N6-hydroxynorvalyl carbamoyladenosine | ms2hn6A | A | Yes |
| 2′-O-methyladenosine | m6A | A | Yes |
| isopentenyladenosine | Iga | A | Yes |
| N6-(cis-hydroxyisopentenyl)adenosine | io6A | A | Yes |
| N6,2′-O-dimethyladenosine | m6Am | A | Yes |
| N⁶,2′-O-dimethyladenosine | m⁶Am | A | Yes |
| N6,N6,2′-O-trimethyladenosine | m62Am | A | Yes |
| N6,N6-dimethyladenosine | m62A | A | Yes |
| N6-acetyladenosine | ac6A | A | Yes |
| N6-hydroxynorvalylcarbamoyladenosine | hn6A | A | Yes |
| N6-methyl-N6-threonylcarbamoyladenosine | m6t6A | A | Yes |
| 2-methyladenosine | m²A | | |
| 2-methylthio-N⁶-isopentenyladenosine | ms²i⁶A | A | Yes |
| N1-methyl-adenosine | — | A | Yes |
| N6,N6-(dimethyl)adenine | — | A | Yes |
| N6-cis-hydroxy-isopentenyladenosine | — | A | Yes |
| α-thio-adenosine | — | A | Yes |
| 2-(amino)adenine | — | A | Yes |
| 2-(aminopropyl)adenine | — | A | Yes |
| 2-(methylthio)-N6-(isopentenyl)adenine | — | A | Yes |
| 2-(alkyl)adenine | — | A | Yes |
| 2-(aminoalkyl)adenine | — | A | Yes |
| 2-(aminopropyl)adenine | — | A | Yes |
| 2-(halo)adenine | — | A | Yes |
| 2-(propyl)adenine | — | A | Yes |
| 2′-Amino-2′-deoxy-ATP | — | A | Yes |
| 2′-Azido-2′-deoxy-ATP | — | A | Yes |
| 2′-Deoxy-2′-a-aminoadenosine TP | — | A | Yes |
| 2′-Deoxy-2′-a-azidoadenosine TP | — | A | Yes |
| 6-(alkyl)adenine | — | A | Yes |
| 6-(methyl)adenine | — | A | Yes |
| 6-(alkyl)adenine | — | A | Yes |
| 6-(methyl)adenine | — | A | Yes |
| 7-(deaza)adenine | — | A | Yes |
| 8-(alkenyl)adenine | — | A | Yes |
| 8-(amino)adenine | — | A | Yes |
| 8-(halo)adenine | — | A | Yes |
| 8-(hydroxyl)adenine | — | A | Yes |
| 8-(thioalkyl)adenine | — | A | Yes |
| 8-(thiol)adenine | — | A | Yes |
| 8-azido-adenosine | — | A | Yes |
| aza adenine | — | A | Yes |
| deaza adenine | — | A | Yes |
| N6-(methyl)adenine | — | A | Yes |
| N6-(isopentyl)adenine | — | A | Yes |
| 7-deaza-8-aza-adenosine | — | A | Yes |
| 7-methyladenine | — | A | NO |
| 1-Deazaadenosine TP | — | A | NO |
| 2′-Fluoro-N6-Bz-deoxyadenosine TP | — | A | NO |
| 2′-OMe-2-Amino-ATP | — | A | NO |
| 2′O-methyl-N6-Bz-deoxyadenosine TP | — | A | NO |
| 2′-a-Ethynyladenosine TP | — | A | NO |
| 2-aminoadenine | — | A | NO |
| 2-Aminoadenosine TP | — | A | NO |
| 2-Amino-ATP | — | A | NO |
| 2′-a-Trifluoromethyladenosine TP | — | A | NO |
| 2-Azidoadenosine TP | — | A | NO |
| 2′-b-Ethynyladenosine TP | — | A | VO |
| 2-Bromoadenosine TP | — | A | NO |
| 2′-b-Trifluoromethyladenosine TP | — | A | NO |
| 2-Chloroadenosine TP | — | A | NO |
| 2′-Deoxy-2′,2′-difluoroadenosine TP | — | A | NO |
| 2′-Deoxy-2′-a-mercaptoadenosine TP | — | A | NO |
| 2′-Deoxy-2′-a-thiomethoxyadenosine TP | — | A | NO |
| 2′-Deoxy-2′-b-aminoadenosine TP | — | A | NO |
| 2′-Deoxy-2′-b-azidoadenosine TP | — | A | NO |
| 2′-Deoxy-2′-b-bromoadenosine TP | — | A | NO |
| 2′-Deoxy-2′-b-chloroadenosine TP | — | A | NO |
| 2′-Deoxy-2′-b-fluoroadenosine TP | — | A | NO |
| 2′-Deoxy-2′-b-iodoadenosine TP | — | A | NO |
| 2′-Deoxy-2′-b-mercaptoadenosine TP | — | A | NO |
| 2′-Deoxy-2′-b-thiomethoxyadenosine TP | — | A | NO |
| 2-Fluoroadenosine TP | — | A | NO |
| 2-lodoadenosine TP | — | A | NO |
| 2-Mercaptoadenosine TP | — | A | NO |
| 2-methoxy-adenine | — | A | NO |
| 2-methylthio-adenine | — | A | NO |
| 2-Trifluoromethyladenosine TP | — | A | NO |
| 3-Deaza-3-bromoadenosine TP | — | A | NO |
| 3-Deaza-3-chloroadenosine TP | — | A | NO |
| 3-Deaza-3-fluoroadenosine TP | — | A | NO |
| 3-Deaza-3-iodoadenosine TP | — | A | NO |
| 3-Deazaadenosine TP | — | A | NO |
| 4′-Azidoadenosine TP | — | A | NO |
| 4′-Carbocyclic adenosine TP | — | A | NO |
| 4′-Ethynyladenosine TP | — | A | NO |
| 5′-Homo-adenosine TP | — | A | NO |
| 8-Aza-ATP | — | A | NO |
| 8-bromo-adenosine TP | — | A | NO |
| 8-Trifluoromethyladenosine TP | — | A | NO |
| 9-Deazaadenosine TP | — | A | NO |
| 2-aminopurine | — | A/G | NO |
| 7-deaza-2,6-diaminopurine | — | A/G | NO |
| 7-deaza-8-aza-2,6-diaminopurine | — | A/G | NO |
| 7-deaza-8-aza-2-aminopurine | — | A/G | NO |
| 2,6-diaminopurine | — | A/G | NO |
| 7-deaza-8-aza-adenine | — | A/G | NO |
| 7-deaza-2-aminopurine | — | A/G | NO |
| 2-thiocytidine | S2C | C | YES |
| 3-methylcytidine | m3C | C | YES |
| 5-formylcytidine | f5C | C | YES |
| 5-hydroxymethylcytidine | hm5C | C | YES |
| 5-methylcytidine | m5C | C | YES |
| N4-acetylcytidine | ac4C | C | YES |
| 2′-O-methylcytidine | Cm | C | YES |
| 2′-O-methylcytidine | Cm | C | YES |
| 5,2′-O-dimethylcytidine | m5Cm | C | YES |
| 5-formyl-2′-O-methylcytidine | f5Cm | C | YES |
| lysidine | k2C | C | YES |
| N4,2′-O-dimethylcytidine | m4Cm | C | YES |
| N4-acetyl-2′-O-methylcytidine | ac4Cm | C | YES |
| N4-methylcytidine | m4C | C | YES |
| N4,N4-dimethyl-2′-OMe-cytidine TP | — | C | YES |
| 4-methylcytidine | — | C | NO |
| 5-aza-cytidine | — | C | NO |
| Pseudo-iso-cytidine | — | C | NO |
| pyrrolo-cytidine | — | C | NO |
| α-thio-cytidine | — | C | NO |
| 2-(thio)cytosine | — | C | NO |
| 2′-Amino-2′-deoxy-CTP | — | C | NO |
| 2′-Azida-2′-deoxy-CTP | — | C | NO |
| 2′-Deoxy-2′-a-aminocytidine TP | — | C | NO |
| 2′-Deoxy-2′-a-azidocytidine TP | — | C | NO |
| 3-(deaza)-5-(aza)cytosine | — | C | NO |
| 3-(methyl)cytosine | — | C | NO |
| 3-(alkyl)cytosine | — | C | NO |
| 3-(deaza)-5-(aza)cytosine | — | C | NO |
| 3-(methyl)cytidine | — | C | NO |
| 4,2′-O-dimethylcytidine | — | C | NO |
| 5-(halo)cytosine | — | C | NO |
| 5-(methyl)cytosine | — | C | NO |
| 5-(propynyl)cytosine | — | C | NO |
| 5-(trifluoromethyl)cytosine | — | C | NO |
| 5-(alkyl)cytosine | — | C | NO |
| 5-(alkynyl)cytosine | — | C | NO |
| 5-(halo)cytosine | — | C | NO |
| 5-(propynyl)cytosine | — | C | NO |
| 5-(trifluoromethyl)cytosine | — | C | NO |
| 5-bromo-cytidine | — | C | NO |
| 5-iodo-cytidine | — | C | NO |
| 5-propynyl cytosine | — | C | NO |
| 6-(azo)cytosine | — | C | NO |
| 6-aza-cytidine | — | C | NO |
| aza cytosine | — | C | NO |
| deaza cytosine | — | C | NO |
| N4 (acetyl)cytosine | — | C | NO |
| 1-methyl-1-deaza-pseudoisocytidine | — | C | NO |
| 1-methyl-pseudoisocytidine | — | C | NO |
| 2-methoxy-5-methyl-cytidine | — | C | NO |
| 2-methoxy-cytidine | — | C | NO |
| 2-thio-5-methyl-cytidine | — | C | NO |
| 4-methoxy-1-methyl-pseudoisocytidine | — | C | NO |
| 4-methoxy-pseudoisocytidine | — | C | NO |
| 4-thio-1-methyl-1-deaza-pseudoisocytidine | — | C | NO |
| 4-thio-1-methyl-pseudoisocytidine | — | C | NO |
| 4-thio-pseudoisocytidine | — | C | NO |
| 5-aza-zebularine | — | C | NO |
| 5-methyl-zebularine | — | C | NO |
| pyrrolo-pseudoisocytidine | — | C | NO |
| zebularine | — | C | NO |
| (E)-5-(2-Broma-vinyl)cytidine TP | — | C | NO |
| 2,2′-anhydro-cytidine-TP-hydrochloride | — | C | NO |
| 2′Fluor-N4-Bz-cytidine TP | — | C | NO |
| 2′Fluoro-N4-Acetyl-cytidine TP | — | C | NO |
| 2′-O-Methyl-N4-Acetyl-cytidine TP | — | C | NO |
| 2′-O-methyl-N4-Bz-cytidine TP | — | C | NO |
| 2′-a-Ethynylcytidine TP | — | C | NO |
| 2′-a-Trifluoromethylcytidine TP | — | C | NO |
| 2′-b-Ethynylcytidine TP | — | C | NO |
| 2′-b-Trifluoromethylcytidine TP | — | C | NO |
| 2′-Deoxy-2′,2′-difluorocytidine TP | — | C | NO |
| 2′-Deoxy-2′-a-mercaptocytidine TP | — | C | NO |
| 2′-Deoxy-2′-a-thiomethoxycytidine TP | — | C | NO |
| 2′-Deoxy-2′-b-aminocytidine TP | — | C | NO |
| 2′-Deoxy-2′-b-azidocytidine TP | — | C | NO |
| 2′-Deoxy-2′-b-bromocytidine TP | — | C | NO |
| 2′-Deoxy-2′-b-chlorocytidine TP | — | C | NO |
| 2′-Deoxy-2′-b-fluorocytidine TP | — | C | NO |
| 2′-Deoxy-2′-b-iodocytidine TP | — | C | NO |
| 2′-Deoxy-2′-b-mercaptocytidine TP | — | C | NO |
| 2′-Deoxy-2′-b-thiomethoxycytidine TP | — | C | NO |
| 2′-O-Methyl-5-(1-propynyl)cytidine TP | — | C | NO |
| 3′-Ethynylcytidine TP | — | C | NO |
| 4′-Azidocytidine TP | — | C | NO |
| 4′-Carbocyclic cytidine TP | — | C | NO |
| 4′-Ethynylcytidine TP | — | C | NO |
| 5-(1-Propynyl)ara-cytidine TP | — | C | NO |
| 5-(2-Chloro-phenyl)-2-thiocytidine TP | — | C | NO |
| 5-(4-Amino-phenyl)-2-thiocytidine TP | — | C | NO |
| 5-Aminoallyl-CTP | — | C | NO |
| 5-Cyanocytidine TP | — | C | NO |
| 5-Ethynylara-cytidine TP | — | C | NO |
| 5-Ethynylcytidine TP | — | C | NO |
| 5′-Homo-cytidine TP | — | C | NO |
| 5-Methoxycytidine TP | — | C | NO |
| 5-Trifluoromethyl-cytidine TP | — | C | NO |
| N4-Amino-cytidine TP | — | C | NO |
| N4-Benzoyl-cytidine TP | — | C | NO |
| 7-methylguanosine | m7G | G | YES |
| N2,2′-O-dimethylguanosine | m2Gm | G | YES |
| N2-methylguanosine | m26 | G | YES |
| wyosme | imG | G | YES |
| 1,2′-O-dimethylguanosine | m1Gm | G | YES |
| 1-methylguanosine | m1G | G | YES |
| 2′-O-ribosylguanosine (phosphate) | Gr(p) | G | YES |
| 2′-O-methylguanosine | Gm | G | YES |
| 7-aminomethyl-7-deazaguanosine | preQ1 | G | YES |
| 7-cyano-7-deazaguanosine | preQ0 | G | YES |
| archaeosine | G+ | G | YES |
| methylwyosine | mimG | G | YES |
| N2,7-dimethylguanosine | m2,7G | G | YES |
| N2,N2,2′-O-trimethylguanosine | m22Gm | G | YES |
| N2,N2,7-trimethylguanosine | m2,2,7G | G | YES |
| N2,N2-dimethylguanosine | m22G | G | YES |
| N²,7,2′-O-trimethylguanosine | m^{2,7}Gm | G | YES |
| 6-thio-guanosine | — | G | NO |
| 7-deaza-guanosine | — | G | NO |
| 8-oxo-guanosine | — | G | NO |
| N1-methyl-guanosine | — | G | NO |
| α-thio-guanosine | — | G | NO |
| 2-(propyl)guanine | — | G | NO |
| 2-(alkyl)guanine | — | G | NO |
| 2′-Amino-2′-deoxy-6TP | — | G | NO |
| 2′-Azido-2′-deoxy-6TP | — | G | NO |
| 2′-Deoxy-2′-a-aminoguanosine TP | — | G | NO |
| 2′-Deoxy-2′-a-azidoguanosine TP | — | G | NO |
| 6-(methyl)guanine | — | G | NO |
| 6-(alkyl)guanine | — | G | NO |
| 6-(methyl)guanine | — | G | NO |
| 6-methyl-guanosine | — | G | NO |
| 7-(alkyl)guanine | — | G | NO |
| 7-(deaza)guanine | — | G | NO |
| 7-(methyl)guanine | — | G | NO |
| 7-(alkyl)guanine | — | G | NO |
| 7-(deaza)guanine | — | G | NO |
| 7-(methyl)guanine | — | G | NO |
| 8-(alkyl)guanine | — | G | NO |
| 8-(alkynyl)guanine | — | G | NO |
| 8-(halo)guanine | — | G | NO |
| 8-(thioalkyl)guanine | — | G | NO |
| 8-(alkenyl)guanine | — | G | NO |
| 8-(alkyl)guanine | — | G | NO |
| 8-(alkynyl)guanine | — | G | NO |
| 8-(amino)guanine | — | G | NO |
| 8-(halo)guanine | — | G | NO |
| 8-(hydroxyl)guanine | — | G | NO |
| 8-(thioalkyl)guanine | — | G | NO |
| 8-(thiol)guanine | — | G | NO |
| azaguamne | — | G | NO |
| deaza guanine | — | G | NO |
| N (methyl)guanine | — | G | NO |
| N-(methyl)guanine | — | G | NO |
| 1-methyl-6-thio-guanosine | — | G | NO |
| 6-methoxy-guanosine | — | G | NO |
| 6-thio-7-deaza-8-aza-guanosine | — | G | NO |
| 6-thio-7-deaza-guanosine | — | G | NO |
| 6-thio-7-methyl-guanosine | — | G | NO |
| 7-deaza-8-aza-guanosine | — | G | NO |
| 7-methyl-8-oxo-guanosine | — | G | NO |
| N2,N2-dimethyl-6-thio-guanosine | — | G | NO |
| N2-methyl-6-thio-guanosine | — | G | VO |
| 1-Me-GTP | — | G | NO |
| 2′Fluoro-N2-isobutyl-guanosine TP | — | G | NO |
| 2′O-methyl-N2-isobutyl-guanosine TP | — | G | NO |
| 2′-a-Ethynylguanosine TP | — | G | NO |
| 2′-a-Trifluoromethylguanosine TP | — | G | NO |
| 2′-b-Ethynylguanosine TP | — | G | NO |
| 2′-b-Trifluoromethylguanosine TP | — | G | NO |
| 2′-Deoxy-2′,2′-difluoroguanosine TP | — | G | NO |
| 2′-Deoxy-2′-a-mercaptoguanosine TP | — | G | NO |
| 2′-Deoxy-2′-a-thiomethoxyguanosine TP | — | G | NO |
| 2′-Deoxy-2′-b-aminoguanosine TP | — | G | NO |
| 2′-Deoxy-2′-b-azidoguanosine TP | — | G | NO |
| 2′-Deoxy-2′-b-bromoguanosine TP | — | G | NO |
| 2′-Deoxy-2′-b-chloroguanosine TP | — | G | NO |
| 2′-Deoxy-2′-b-fluoroguanosine TP | — | G | NO |
| 2′-Deoxy-2′-b-iodoguanosine TP | — | G | NO |
| 2′-Deoxy-2′-b-mercaptoguanosine TP | — | G | NO |
| 2′-Deoxy-2′-b-thiomethoxyguanosine TP | — | G | NO |
| 4′-Azidoguanosine TP | — | G | NO |
| 4′-Carbocyclic guanosine TP | — | G | NO |
| 4′-Ethynylguanosine TP | — | G | NO |
| 5′-Homo-guanosine TP | — | G | NO |
| 8-bromo-guanosine TP | — | G | NO |
| 9-Deazaguanosine TP | — | G | NO |
| N2-isobutyl-guanosin TP | — | G | NO |
| 1-methylinosine | m1l | I | YES |
| mosme | I | I | YES |
| 1,2′-O-dimethylinosine | m1lm | I | YES |
| 7-methylinosine | — | I | NO |
| 2′-O-methylinosine | 1m | I | YES |
| epoxyqueuosme | oQ | Q | YES |
| galactosyl-queuosine | galQ | Q | YES |
| mannosylqueuosine | manQ | Q | YES |
| queuosme | Q | Q | YES |
| allyamino-thymidine | — | T | NO |
| aza thymidine | — | T | NO |
| deaza thymidine | — | T | NO |
| deoxy-thymidine | — | T | NO |
| 2-thiouridine | s2U | U | YES |
| 3-methyluridine | m3U | U | YES |
| 5-carboxymethyluridine | cm5U | U | YES |
| 5-hydroxyuridine | ho5L | U | YES |
| 5-methyluridine | m5U | U | YES |
| 5-taurinomethyl-2-thiouridine | τm5s2U | U | YES |
| 5-taurinomethyluridine | τm5U | U | YES |
| dihydrouridine | D | U | YES |
| pseudouridine | Ψ | U | YES |
| (3-(3-amino-5-carboxypropyl)uridine | acp3U | | |
| 1-methyl-3-(3-amino-5-carboxypropyl)pseudouridine | mlacp3Ψ | U | YES |
| 1-methylpseudouridine | mlΨ | U | YES |
| 1-methyl-pseudouridine | mlΨ | U | YES |
| 2′-O-methylpseudouridine | Ψm | U | YES |
| 2′-O-methyluridine | Ψm | U | YES |
| 2-thio-2′-O-methyluridine | s2Um | U | YES |
| 3-(3-amino-3-carboxypropyl)uridine | acp3U | U | YES |
| 3,2′-O-dimethyluridine | m3Um | U | YES |
| 3-methyl-pseudouridine TP | — | U | YES |
| 4-thiouridine | s4U | U | YES |
| 5-(carboxyhydroxymethyl)uridine | chm5U | U | YES |
| 5-(carboxyhydroxymethyl)uridine methyl ester | mchm5U | U | YES |
| 5,2′-O-dimethyluridine | m5Um | U | YES |
| 5,6-dihydro-uridine | — | U | YES |
| 5-aminomethyl-2-thiouridine | nm5s2U | U | YES |
| 5-carbamoylmethyl-2′-O-methyluridine | ncm5Um | U | YES |
| 5-carbamoylmethyluridine | ncm5U | U | YES |
| 5-carboxyhydroxymethyluridine | — | U | YES |
| 5-carboxyhydroxymethyluridine methyl ester | — | U | YES |
| 5-carboxymethylaminomethyl-2′-O-methyluridine | cmnm5Um | U | YES |
| 5-carboxymethylaminomethyl-2-thiouridine | cmnm5s2U | U | YES |
| 5-carboxymethylaminomethyluridine | cmnm5U | U | YES |
| 5-Carbamoylmethyluridine TP | — | U | YES |
| 5-methoxycarbonylmethyl-2′-O-methyluridine | mcm5Um | U | YES |
| 5-methoxycarbonylmethyl-2-thiouridine | mcm5s2U | U | YES |
| 5-methoxycarbonylmethyluridine | mcm5U | U | YES |
| 5-methoxyuridine | mo5U | U | YES |
| 5-methyl-2-thiouridine | m5s2U | U | YES |
| 5-methylaminomethyl-2-selenouridine | mnm5se2U | U | YES |
| 5-methylaminomethyl-2-thiouridine | mnm5s2U | U | YES |
| 5-methylaminomethyluridine | mnm5U | U | YES |
| 5-Methyldihydrouridine | — | U | YES |
| 5-Oxyacetic acid-uridine TP | — | U | YES |
| 5-Oxyacetic acid-methyl ester-uridine TP | — | U | YES |
| N1-methyl-pseudo-uridine | — | U | YES |
| Uridine-5-oxyacetic acid | cmo5U | U | YES |
| uridine 5-oxyacetic acid methyl ester | mcmo5U | U | YES |
| 3-(3-Amino-3-carboxypropyl)-uridine TP | — | U | YES |
| 5-(iso-Pentenylaminomethyl)-2-thiouridine TP | — | U | YES |
| 5-(iso-Pentenylaminomethyl)-2′-O-methyluridine TP | — | U | YES |
| 5-(iso-Pentenylaminomethyl)uridine TP | — | U | YES |
| 5-propynyluracil | — | U | NO |
| α-thio-uridine | — | U | NO |
| 1-(aminoalkylamino-carbonylethylenyl)-2(thio)-pseudouracil | — | U | NO |
| 1-(aminoalkylaminocarbonylethylenyl)-2,4-(dithio)pseudouracil | — | U | NO |
| 1-(aminoalkylaminocarbonylethylenyl)-4(thio)pseudouracil | — | U | NO |
| 1-(aminoalkylaminocarbonylethylenyl)-pseudouracil | — | U | NO |
| 1-(aminocarbonylethylenyl)-2(thio)-pseudouracil | — | U | NO |
| 1-(aminocarbonylethylenyl)-2,4-(dithio)pseudouracil | — | U | NO |
| 1-(aminocarbonylethylenyl)-4(thio)pseudouracil | — | U | NO |
| 1-(aminocarbonylethylenyl)-pseudouracil | — | U | NO |
| 1 substituted 2(thio)-pseudouracil | — | U | NO |
| 1 substituted 2,4-(dithio)pseudouracil | — | U | NO |
| 1 substituted 4 (thio)pseudouracil | — | U | NO |
| 1 substituted pseudouracil | — | U | NO |
| 1-(aminoalkylamino-carbonylethylenyl)-2-(thio)-pseudouracil | — | U | NO |
| 1-Methyl-3-(3-amino-3-carboxypropyl)pseudouridine TP | — | U | NO |
| 1-Methyl-3-(3-amino-3-carboxypropyl)pseudo-UTP | — | U | NO |
| 1-Methyl-pseudo-UTP | — | U | NO |
| 2-(thio)pseudouracil | — | U | NO |
| 2′-deoxyuridine | 2′dU | U | NO |
| 2′-fluorouridine | — | U | NO |
| 2-(thia)uracil | — | U | NO |
| 2,4-(dithio)psuedouracil | — | U | NO |
| 2′-methyl-2′-amino-2′-azido-2′fluro-guanosine | — | U | NO |
| 2′-Amino-2′-deoxy-UTP | — | U | NO |
| 2′-Azido-2′-deoxy-UTP | — | U | NO |
| 2′-Azido-deoxyuridine TP | — | U | NO |
| 2′-O-methylpseudouridine | — | U | NO |
| 2′-Deoxy-2′-a-aminouridine TP | — | U | NO |
| 2′-Deoxy-2′-a-azidouridine TP | — | U | NO |
| 2-methylpseudouridine | m3Ψ | U | VO |
| 3-(3-amino-3-carboxypropyl)uracil | — | U | NO |
| 4-(thia)pseudouracil | — | U | NO |
| 4-(thio)pseudouracil | — | U | NO |
| 4-(thio)uracil | — | U | NO |
| 4-thiouracil | — | U | NO |
| 5-(1,3-diazole-1-alkyl)uracil | — | U | NO |
| 5-(2-aminopropyl)uracil | — | U | NO |
| 5-(aminoalkyl)uracil | — | U | NO |
| 5-(dimethylaminoalkyl)uracil | — | U | NO |
| 5-(guanidiniumalkyl)uracil | — | U | NO |
| 5-(methoxycarbonylmethyl)-2-(thia)uracil | — | U | NO |
| 5-(methoxycarbonyl-methyl)uracil | — | U | NO |
| 5-(methyl)-2-(this)uracil | — | U | NO |
| 5-(methyl)-2,4-(dithio)uracil | — | U | NO |
| 5-(methyl)-4-(thia)uracil | — | U | NO |
| 5-(methylaminomethyl)-2-(thin)uracil | — | U | NO |
| 5-(methylaminomethyl)-2,4-(dithio)uracil | — | U | NO |
| 5-(methylaminomethyl)-4-(thio)uracil | — | U | NO |
| 5-(propynyl)uracil | — | U | NO |
| 5-(trifluoromethyl)uracil | — | U | NO |
| 5-(2-aminopropyl)uracil | — | U | VO |
| 5-(alkyl)-2-(this)pseudouracil | — | U | NO |
| 5-(alkyl)-2,4-(dithio)pseudouracil | — | U | NO |
| 5-(alkyl)-4-(thio)pseudouracil | — | U | NO |
| 5-(alkyl)pseudouracil | — | U | NO |
| 5-(alkyl)uracil | — | U | NO |
| 5-(alkynyl)uracil | — | U | NO |
| 5-(allylamino)uracil | — | U | NO |
| 5-(cyanoalkyl)uracil | — | U | NO |
| 5-(dialkylaminoalkyl)uracil | — | U | NO |
| 5-(dimethylaminoalkyl)uracil | — | U | NO |
| 5-(guanidiniumalkyl)uracil | — | U | NO |
| 5-(halo)uracil | — | U | NO |
| 5-(1,3-diazole-1-alkyl)uracil | — | U | NO |
| 5-(methoxy)uracil | — | U | NO |
| 5-(methoxycarbonylmethyl)-2-(thio)uracil | — | U | NO |
| 5-(methoxycarbony-1-methyl)uracil | — | U | NO |
| 5-(methyl)-2(thia)uracil | — | U | NO |
| 5-(methyl)-2,4-(dithio)uracil | — | U | NO |
| 5-(methyl)-4-(thia)uracil | — | U | NO |
| 5-(methyl)-2-(thia)pseudouracil | — | U | NO |
| 5-(methyl)-2,4-(dithio)pseudouracil | — | U | NO |
| 5-(methyl)-4-(thio)pseudouracil | — | U | NO |
| 5-(methyl)pseudouracil | — | U | NO |
| 5-(methylaminomethyl)-2-(thio)uracil | — | U | NO |
| 5-(methylaminomethyl)-2,4(dithia)uracil | — | U | NO |
| 5-(methylaminomethyl)-4-(this)uracil | — | U | NO |
| 5-(propynyl)uracil | — | U | NO |
| 5-(trifluoromethyl)uracil | — | U | NO |
| 5-aminoally-1-uridine | — | U | NO |
| 5-bromo-uridine | — | U | NO |
| 5-iodo-uridine | — | U | NO |
| 5-uracil | — | U | NO |
| 6-(azo)uracil | — | U | NO |
| 6-(azo)uracil | — | U | NO |
| 6-aza-uridine | — | U | NO |
| allyamino-uracil | — | U | NO |
| aza uracil | — | U | NO |
| deaza uracil | — | U | NO |
| N3-(methyl)uracil | — | U | NO |
| Pseudo-UTP-1-2-ethanoic | — | U | NO |
| pseudouracil | — | U | NO |
| 4-Thio-pseudo-UTP | — | U | NO |
| 1-carboxymethyl-pseudouridine | — | U | NO |
| 1-methyl-1-deaza-pseudouridine | — | U | NO |
| 1-propynyl-uridine | — | U | NO |
| 1-taurinomethyl-1-methyl-uridine | — | U | NO |
| 1-taurinomethyl-4-thio-uridine | — | U | NO |
| 1-taurinomethy-l-pseudouridine | — | U | NO |
| 2-methoxy-4-thio-pseudouridine | — | U | NO |
| 2-thio-1-methyl-1-deaza-pseudouridine | — | U | VO |
| 2-thio-1-methyl-pseudouridine | — | U | NO |
| 2-thio-5-aza-uridine | — | U | NO |
| 2-thio-dihydropseudouridine | — | U | NO |
| 2-thin-dihydrouridine | — | U | NO |
| 2-thio-pseudouridine | — | U | NO |
| 4-methoxy-2-thio-pseudouridine | — | U | NO |
| 4-methoxy-pseudouridine | — | U | NO |
| 4-thio-1-methyl-pseudouridine | — | U | NO |
| 4-thin-pseudouridine | — | U | NO |
| 5-aza-uridine | — | U | NO |
| dihydropseudouridine | — | U | NO |
| (±)1-(2-Hydroxypropyl)pseudouridine TP | — | U | NO |
| (2R)-1-(2-Hydroxypropyl)pseudouridine TP | — | U | NO |
| (2S)-1-(2-Hydroxypropyl)pseudouridine TP | — | U | NO |
| €-5-(2-Bromo-vinyl)ara-uridine TP | — | U | NO |
| €-5-(2-Bromo-vinyl)uridine TP | — | U | NO |
| (2)-5-(2-Bromo-vinyl)ara-uridine TP | — | U | NO |
| (Z)-5-(2-Bromo-vinyl)uridine TP | — | U | NO |
| 1-(2,2,2-Trifluoroethyl)-pseudo-UTP | — | U | NO |
| 1-(2,2,3,3,3-Pentafluoropropyl)pseudouridine TP | — | U | NO |
| 1-(2,2-Diethoxyethyl)pseudouridine TP | — | U | NO |
| 1-(2,4,6-Trimethylbenzyl)pseudouridine TP | — | U | NO |
| 1-(2,4,6-Trimethyl-benzyl)pseudo-UTP | — | U | NO |
| 1-(2,4,6-Trimethyl-phenyl)pseudo-UTP | — | U | NO |
| 1-(2-Amino-2-carboxyethyl)pseudo-UTP | — | U | NO |
| 1-(2-Amino-ethyl)pseudo-UTP | — | U | NO |
| 1-(2-Hydroxyethyl)pseudouridine TP | — | U | NO |
| 1-(2-Methoxyethyl)pseudouridine TP | — | U | NO |
| 1-(3,4-Bis-trifluoromethoxybenzyl)pseudouridine TP | — | U | NO |
| 1-(3,4-Dimethoxybenzyl)pseudouridine TP | — | U | NO |
| 1-(3-Amino-3-carboxypropyl)pseudo-UTP | — | U | NO |
| 1-(3-Amino-propyl)pseudo-UTP | — | U | NO |
| 1-(3-Cyclopropyl-prop-2-ynyl)pseudouridine TP | — | U | NO |
| 1-(4-Amino-4-carboxybutyl)pseudo-UTP | — | U | NO |
| 1-(4-Amino-benzyl)pseudo-UTP | — | U | NO |
| 1-(4-Amino-butyl)pseudo-UTP | — | U | NO |
| 1-(4-Amino-phenyl)pseudo-UTP | — | U | NO |
| 1-(4-Azidobenzyl)pseudouridine TP | — | U | NO |
| 1-(4-Bromobenzyl)pseudouridine TP | — | U | NO |
| 1-(4-Chlorobenzyl)pseudouridine TP | — | U | NO |
| 1-(4-Fluorobenzyl)pseudouridine TP | — | U | NO |
| 1-(4-lodobenzyl)pseudouridine TP | — | U | NO |
| 1-(4-Methanesulfonylbenzyl)pseudouridine TP | — | U | NO |
| 1-(4-Methoxybenzyl)pseudouridine TP | — | U | NO |
| 1-(4-Methoxy-benzyl)pseudo-UTP | — | U | NO |
| 1-(4-Methoxy-phenyl)pseudo-UTP | — | U | NO |
| 1-(4-Methylbenzyl)pseudouridine TP | — | U | NO |
| 1-(4-Methyl-benzyl)pseudo-UTP | — | U | NO |
| 1-(4-Nitrobenzyl)pseudouridine TP | — | U | NO |
| 1-(4-Nitro-benzyl)pseudo-UTP | — | U | NO |
| 1-(4-Nitro-phenyl)pseudo-UTP | — | U | NO |
| 1-(4-Thiomethoxybenzyl)pseudouridine TP | — | U | NO |
| 1-(4-Trifluoromethoxybenzyl)pseudouridine TP | — | U | NO |
| 1-(4-Trifluoromethyibenzyl)pseudouridine TP | — | U | NO |
| 1-(5-Amino-pentyl)pseudo-UTP | — | U | NO |
| 1-(6-Amino-hexyl)pseudo-UTP | — | U | NO |
| 1,6-Dimethyl-pseudo-UTP | — | U | NO |
| 1-[3-(2-{2-[2-(2-Aminoethoxy)-ethoxy]-ethoxy}-ethoxy)- | — | U | NO |
| propiony]pseudouridine TP | — | U | NO |
| 1-{3-[2-(2-Aminoethoxy)-ethoxy]-propiony/}pseudouridine TP | — | U | NO |
| 1-Acetylpseudouridine TP | — | U | NO |
| 1-Alkyl-6-(1-propynyl)-pseudo-UTP | — | U | NO |
| 1-Alkyl-6-(2-propynyl)-pseudo-UTP | — | U | NO |
| 1-Alkyl-6-allyl-pseudo-UTP | — | U | NO |
| 1-Alkyl-6-ethynyl-pseudo-UTP | — | U | NO |
| 1-Alkyl-6-homoallyl-pseudo-UTP | — | U | NO |
| 1-Alkyl-6-vinyl-pseudo-UTP | — | U | NO |
| 1-Allylpseudouridine TP | — | U | NO |
| 1-Aminornethyl-pseudo-UTP | — | U | NO |
| 1-Benzoylpseudouridine TP | — | U | NO |
| 1-Benzyloxymethylpseudouridine TP | — | U | NO |
| 1-Benzyl-pseudo-UTP | — | U | NO |
| 1-Biotinyl-PE62-pseudouridine TP | — | U | NO |
| 1-Biotinylpseudouridine TP | — | U | NO |
| 1-Butyl-pseudo-UTP | — | U | NO |
| 1-Cyanomethylpseudouridine TP | — | U | NO |
| 1-Cyclobutylmethyl-pseudo-UTP | — | U | NO |
| 1-Cyclobutyl-pseudo-UTP | — | U | NO |
| 1-Cycloheptylmethyl-pseudo-UTP | — | U | NO |
| 1-Cycloheptyl-pseudo-UTP | — | U | NO |
| 1-Cyclohexylmethyl-pseudo-UTP | — | U | NO |
| 1-Cyclohexyl-pseudo-UTP | — | U | NO |
| 1-Cyclooctylmethyl-pseudo-UTP | — | U | NO |
| 1-Cyclooctyl-pseudo-UTP | — | U | NO |
| 1-Cyclopentylmethyl-pseudo-UTP | — | U | NO |
| 1-Cyclopentyl-pseudo-UTP | — | U | NO |
| 1-Cyciopropyimethyl-pseudo-UTP | — | U | NO |
| 1-Cyclopropyl-pseudo-UTP | — | U | NO |
| 1-Ethyl-pseudo-UTP | — | U | NO |
| 1-Hexyl-pseudo-UTP | — | U | NO |
| 1-Homoallylpseudouridine TP | — | D | NO |
| 1-Hydroxymethylpseudouridine TP | — | D | NO |
| 1-iso-propyl-pseudo-UTP | — | D | NO |
| 1-Me-2-thio-pseudo-UTP | — | D | NO |
| 1-Me-4-thio-pseudo-UTP | — | D | NO |
| 1-Me-alpha-thio-pseudo-UTP | — | D | NO |
| 1-Methanesuifonyimethyipseudouridine TP | — | D | NO |
| 1-Methoxymethylpseudouridine TP | — | D | NO |
| 1-Methyl-6-(2,2,2-Trifluoroethyl)pseudo-UTP | — | D | NO |
| 1-Methyl-6-(4-morphoiino)-pseudo-UTP | — | D | NO |
| 1-Methyl-6-(4-thiomorphoiino)-pseudo-UTP | — | D | NO |
| 1-Methyl-6-(substituted phenyl)pseudo-UTP | — | D | NO |
| 1-Methyl-6-amino-pseudo-UTP | — | D | NO |
| 1Methyl-6-azido-pseudo-UTP | — | D | VO |
| 1-Methyl-6-bromo-pseudo-UTP | — | D | NO |
| 1-Methyl-6-butyl-pseudo-UTP | — | D | NO |
| 1-Methyl-6-chioro-pseudo-UTP | — | D | NO |
| 1-Methyl-6-cyano-pseudo-UTP | — | D | NO |
| 1-Methyl-6-dimethyiamino-pseudo-UTP | — | D | NO |
| 1-Methyl-6-ethoxy-pseudo-UTP | — | D | NO |
| 1-Methyl-6-ethylcarboxyiate-pseudo-UTP | — | D | NO |
| 1-Methyl-6-ethyl-pseudo-UTP | — | D | NO |
| 1-Methyl-6-fluoro-pseudo-UTP | — | D | NO |
| 1-Methyl-6-formyl-pseudo-UTP | — | D | NO |
| 1-Methyl-6-hydroxyamino-pseudo-UTP | — | D | NO |
| 1-Methyl-6-hydroxy-pseudo-UTP | — | D | NO |
| 1-Methyl-6-iodo-pseudo-UTP | — | D | NO |
| 1-Methyl-6-iso-propyl-pseudo-UTP | — | D | NO |
| 1-Methyl-6-methoxy-pseudo-UTP | — | D | NO |
| 1-Methyl-6-methylamino-pseudo-UTP | — | D | NO |
| 1-Methyl-6-phenyl-pseudo-UTP | — | D | NO |
| 1-Methyl-o-propyl-pseudo-UTP | — | D | NO |
| 1-Methyl-6-tert-butyl-pseudo-UTP | — | D | NO |
| 1-Methyl-6-trifluoromethoxy-pseudo-UTP | — | D | NO |
| 1-Methyl-6-trifluoromethyl-pseudo-UTP | — | D | NO |
| 1-Morphoiinomethyipseudouridine TP | — | D | NO |
| 1-Pentyl-pseudo-UTP | — | D | VO |
| 1-Phenyl-pseudo-UTP | — | D | NO |
| 1-Pivaloylpseudouridine TP | — | D | NO |
| 1-Propargylpseudouridine TP | — | D | NO |
| 1-Propyl-pseudo-UTP | — | D | NO |
| 1-propynyl-pseudouridine | — | D | NO |
| 1-p-tolyl-pseudo-UTP | — | D | NO |
| 1-tert-Butyl-pseudo-UTP | — | U | NO |
| 1-Thiomethoxymethyipseudouridine TP | — | U | NO |
| 1-Thiomorphoiinomethyipseudouridine TP | — | U | NO |
| 1-Trifluoroacetylpseudouridine TP | — | U | NO |
| 1-Trifluoromethyl-pseudo-UTP | — | U | NO |
| 1-Vinylpseudouridine TP | — | U | NO |
| 2,2′-anhydro-uridine TP | — | U | NO |
| 2′-bromo-deoxyuridine TP | — | U | NO |
| 2′-F-5-Methyl-2′-deoxy-UTP | — | U | NO |
| 2′-Ome-5-Me-UTP | — | U | VO |
| 2′-Ome-pseudo-UTP | — | U | NO |
| 2′-a-Ethynyiuridine TP | — | U | NO |
| 2′-a-Trifluoromethyiuridine TP | — | U | NO |
| 2′-b-Ethynyiuridine TP | — | U | NO |
| 2′-b-Trifluoromethyiuridine TP | — | U | NO |
| 2′-Deoxy-2′,2′-difluorouridine TP | — | U | NO |
| 2′-Deoxy-2′-a-mercaptouridine TP | — | U | NO |
| 2′-Deoxy-2′-a-thiomethoxyuridine TP | — | U | NO |
| 2′-Deoxy-2′-b-aminouridine TP | — | U | NO |
| 2′-Deoxy-2′-b-azidouridine TP | — | U | NO |
| 2′-Deoxy-2′-b-bromouridine TP | — | U | NO |
| 2′-Deoxy-2′-b-chiorouridine TP | — | U | NO |
| 2′-Deoxy-2′-b-fluorouridine TP | — | U | NO |
| 2′-Deoxy-2′-b-iodouridine TP | — | U | NO |
| 2′-Deoxy-2′-b-mercaptouridine TP | — | U | NO |
| 2′-Deoxy-2′-b-thiomethoxyuridine TP | — | U | NO |
| 2-methoxy-4-thio-uridine | — | U | NO |
| 2-methoxyuridine | — | U | NO |
| 2′-O-Methyl-5-(1-propynyl)uridine TP | — | U | NO |
| 3-Alkyl-pseudo-UTP | — | U | NO |
| 4′-Azidouridine TP | — | U | NO |
| 4′-Carbocyclic uridine TP | — | U | NO |
| 4′-Ethynyiuridine TP | — | U | NO |
| 5-(1-Propynyl)ara-uridine TP | — | U | NO |
| 5-(2-Furanyl)uridine TP | — | U | NO |
| 5-Cyanouridine TP | — | U | NO |
| 5-Dimethylaminouridine TP | — | U | NO |
| 5′-Homo-uridine TP | — | U | NO |
| 5-iodo-2′-fluoro-deoxyuridine TP | — | U | NO |
| 5-Phenyiethynyiuridine TP | — | U | NO |
| 5-Trideuteromethyl-6-deuterouridine TP | — | U | NO |
| 5-Trifluoromethyl-Uridine TP | — | U | NO |
| 5-Vinyiarauridine TP | — | U | NO |
| 6-(4-Morpholino)-pseudo-UTP | — | D | NO |
| 6-(4-Thinmorpholina)-pseudo-UTP | — | D | NO |
| 6-(Substituted-Phenyl)-pseudo-UTP | — | D | NO |
| 6-Amino-pseudo-UTP | — | D | NO |
| 6-Azido-pseudo-UTP | — | D | NO |
| 6-Bromo-pseudo-UTP | — | D | NO |
| 6-Butyl-pseudo-UTP | — | D | NO |
| 6-Chloro-pseudo-UTP | — | D | NO |
| 6-Cyano-pseudo-UTP | — | D | NO |
| 6-Dimethylamino-pseudo-UTP | — | D | NO |
| 6-Ethoxy-pseudo-UTP | — | D | NO |
| 6-Ethylcarboxylate-pseudo-UTP | — | D | NO |
| 6-Ethyl-pseudo-UTP | — | D | NO |
| 6-Fluoro-pseudo-UTP | — | D | NO |
| 6-Formyl-pseudo-UTP | — | D | NO |
| 6-Hydroxyamino-pseudo-UTP | — | D | NO |
| 6-Hydroxy-pseudo-UTP | — | D | NO |
| 6-lodo-pseudo-UTP | — | D | NO |
| 6-iso-Propyl-pseudo-UTP | — | D | NO |
| 6-Methoxy-pseudo-UTP | — | D | NO |
| 6-Methylamino-pseudo-UTP | — | D | NO |
| 6-Methyl-pseudo-UTP | — | D | NO |
| 6-Phenyl-pseudo-UTP | — | D | NO |
| 6-Phenyl-pseudo-UTP | — | D | NO |
| 6-Propyl-pseudo-UTP | — | D | NO |
| 6-tert-Butyl-pseudo-UTP | — | D | NO |
| 6-Trifluoromethoxy-pseudo-UTP | — | D | NO |
| 6-Trifluoromethyl-pseudo-UTP | — | D | NO |
| Alpha-thio-pseudo-UTP | — | D | NO |
| Pseudouridine-1-(4-methylbenzenesulfonic acid)TP | — | D | NO |
| Pseudouridine-1-(4-methylbenzoic acid)TP | — | D | NO |
| Pseudouridine-TP-1-[3-(2-ethoxy)]propionic acid | — | D | NO |
| Pseudouridine-TP-1-[3-{2-(2-[2-(2-ethoxy)-ethoxv]-ethoxv)- | — | D | NO |
| ethoxy}]propionic acid | | | |
| Pseudouridine-TP-1-[3-{2-(2-[2-{2(2-ethoxy)-ethoxy}-ethoxy]-ethoxy)- | — | D | NO |
| ethoxy}]propionic acid | | | |
| Pseudouridine TP-1-[3-{2-(2-[2-ethoxy]-ethoxvr-ethoxy}]propionic acid | — | D | NO |
| Pseudouridine-TP-1-[3-{2-(2-ethoxy)-ethoxy}]propionic acid | — | D | NO |
| Pseudouridine-TP-1-methylphosphonic acid | — | D | NO |
| Pseudouridine-TP-1-methylphosphonic acid diethyl ester | — | U | NO |
| Pseudo-UTP-N1-3-propionic acid | — | U | NO |
| Pseudo-UTP-N1-4-butanoicacid | — | U | NO |
| Pseudo-UTP-N1-5-pentanoic acid | — | U | NO |
| Pseudo-UTP-N1-6-hexanoicacid | — | U | NO |
| Pseudo-UTP-N1-7-heptanoic acid | — | U | NO |
| Pseudo-UTP-N1-methyl-p-benzoic acid | — | U | NO |
| Pseudo-UTP-N1-p-benzoic acid | — | U | NO |
| wybutosine | yW | W | Yes |
| hydroxywybutosine | OhyW | W | Yes |
| Isowyosine | imG2 | W | Yes |
| peroxywybutosine | o2yW | W | Yes |
| undermodified hydroxywybutosine | OHyW* | W | Yes |
| 4-demethylwyosine | imG-14 | W | Yes |

Other modifications which may be useful in the polynucleotides of the present invention are listed in Table B1

TABLE B1
Additional modification types

| Name | Type |
|---|---|
| 2,6-(diamino)purine | Other |
| 1-(aza)-2-(thio)-3-(aza)-phenoxazin-1-yl | Other |
| 1,3-(diaza)-2-(oxo)-phenthiazin-1-yl | Other |
| 1,3-(diaza)-2-(oxo)-phenoxazin-1-yl | Other |
| 1,3,5-(triaza)-2,6-(dioxa)-naphthalene | Other |
| 2-(amino)purine | Other |
| 2,4,5-(trimethyl)phenyl | Other |
| 2′ methyl, 2′amino, 2′azido, 2′fluro-cytidine | Other |
| 2′ methyl, 2′amino, 2′azido, 2′fluro-adenine | Other |
| 2′methyl, 2′amino, 2′azido, 2′fluro-uridine | Other |
| 2′-amino-2′-deoxyribose | Other |
| 2-amino-6-chloro-purine | Other |
| 2-aza-inosinyl | Other |
| 2′-azido-2′-deoxyribose | Other |
| 2′-fluoro-Z′-deoxyribose | Other |
| 2′-fluoro-modified bases | Other |
| 2′-O-methyl-ribose | Other |
| 2-oxo-7-aminopyridopyrimidin-3-yl | Other |
| 2-oxo-pyridopyrimidine-3-yl | Other |
| 2-pyridinone | Other |
| 3-nitropyrrole | Other |
| 3-(methyl)-7-(propynyl)isocarbostyrilyl | Other |
| 3-(methyl)isocarbostyrilyl | Other |
| 4-(fluoro)-6-(methyl)benzimidazole | Other |
| 4-(methyl)benzimidazole | Other |
| 4-(methyl)indalyl | Other |
| 4,6-(dimethyl)indalyl | Other |
| 5-nitroindole | Other |
| 5-substituted pyrimidines | Other |
| 5-(methyl)isocarbostyrilyl | Other |
| 5-nitroindole | Other |
| 6-(aza)pyrimidine | Other |
| 6-(azo)thymine | Other |
| 6-(methyl)-7-(aza)indolyl | Other |
| 6-chloro-purine | Other |
| 6-phenyl-pyrrolo-pyrimidin-2-on-3-yl | Other |
| 7-(aminoalkylhydroxy)-1-(aza)-2-(thio)-3-(aza)-phenthiazin-1-yl | Other |
| 7-(aminoalkylhydroxy)-1-(aza)-2-(this)-3-(aza)-phenoxazin-1-yl | Other |
| 7-(aminoalkylhydroxy)-1,3-(diaza)-2-(oxo)-phenoxazin-1-yl | Other |
| 7-(aminoalkylhydroxy)-1,3-(diaza)-2-(oxo)-phenthiazin-1-yl | Other |
| 7-(aza)indolyl | Other |
| 7-(guanidiniumalkylhydroxy)-1-(aza)-2-(this)-3-(aza)-phenoxazin-1-yl | Other |
| 7-(guanidiniumalkylhydroxy)-1-(aza)-2-(thio)-3-(aza)-phenthiazin-1-yl | Other |
| 7-(guanidiniumalkylhydroxy)-1-(aza)-2-(thio)-3-(aza)-phenoxazin-1-yl | Other |
| 7-(guanidiniumalkylhydroxy)-1,3-(diaza)-2-(oxo)-phenoxazin-1-yl | Other |
| 7-(guanidiniumalkyl-hydroxy)-1,3-(diaza)-2-(oxo)-phenthiazin-1-yl | Other |
| 7-(guanidiniumalkylhydroxy)-1,3-(diaza)-2-(oxo)-phenoxazin-1-yl | Other |
| 7-(propynyl)isocarbostyrilyl | Other |
| 7-(propynyl)isocarbostyrilyl, propynyl-7-(aza)indolyl | Other |
| 7-deaza-inosinyl | Other |
| 7-substituted 1-(aza)-2-(thio)-3-(aza)-phenoxazin-1-yl | Other |
| 7-substituted 1,3-(diaza)-2-(oxo)-phenoxazin-1-yl | Other |
| 9-(methyl)-imidizopyridinyl | Other |
| aminoindolyl | Other |
| anthracenyl | Other |
| bis-ortho-(aminoalkylhydroxy)-6-phenyl-pyrrolo-pyrimidin-2-on-3-yl | Other |
| bis-ortho-substituted-6-phenyl-pyrrolo-pyrimidin-2-on-3-yl | Other |
| difluorotolyl | Other |
| hypoxanthine | Other |
| imidizopyridinyl | Other |
| inosinyl | Other |
| isocarbostyrilyl | Other |
| isoguamsme | Other |
| N2-substituted purines | Other |
| N6-methyl-2-amino-purine | Other |
| 6-substituted purines | Other |
| N-alkylated derivative | Other |
| napthalenyl | Other |
| nitrobenzimidazolyl | Other |
| nitroimidazolyl | Other |
| nitroindazolyl | Other |
| nitropyrazolyl | Other |
| nubularine | Other |
| O6-substituted purines | Other |
| O-alkylated derivative | Other |
| ortho-(aminoalkylhydroxy)-6-phenyl-pyrrolo-pyrimidin-2-on-3-yl | Other |
| ortho-substituted-6-phenyl-pyrrolo-pyrimidin-2-on-3-yl | Other |
| Oxoformycin TP | Other |
| para-(aminoalkylhydroxy)-6-phenyl-pyrrolo-pyrimidin-2-on-3-yl | Other |
| para-substituted-6-phenyl-pyrrolo-pyrimidin-2-on-3-yl | Other |
| pentacenyl | Other |
| phenanthracenyl | Other |
| phenyl | Other |
| propynyl-7-(aza)indolyl | Other |
| pyrenyl | Other |
| pyridopyrimidin-3-yl | Other |
| pyridopyrimidin-3-yl, 2-oxo-7-amino-pyridopyrimidin-3-yl | Other |
| pyrrolo-pyrimidin-2-on-3-yl | Other |
| pyrrolopyrimidinyl | Other |
| pyrrolopyrizinyl | Other |
| stilbenzyl | Other |
| substituted 1,2,4-triazoles | Other |
| tetracenyl | Ither |
| tubercidine | Other |
| xanthine | Other |
| Xanthosine-5′-TP | Other |
| 2-thio-zebularine | Other |
| 5-aza-2-thio-zebularine | Other |
| 7-deaza-2-amino-purine | Other |
| pyridin-4-one ribonucleoside | Other |
| 2-Amino-riboside TP | Other |
| Formycin A TP | Other |
| Formycin B TP | Other |
| Pyrrolosine TP | Other |
| 2′-OH-ara-adenosine TP | Other |
| 2′-OH-ara-cytidine TP | Other |
| 2′-OH-ara-uridine TP | Other |
| 2′-OH-ara-guanosine TP | Other |
| 5-(2-carbomethoxyvinyl)uridine TP | Other |
| N6-(19-Amino-pentaoxanonadecyl)adenosine TP | Other |

The polynucleotides can include any useful linker between the nucleosides. Such linkers, including backbone modifications are given in Table B2.

TABLE B2
Linker modifications

| Name | Type |
|---|---|
| 3′-alkylene phosphonates | Linker |
| 3′-amino phosphoramidate | Linker |
| alkene containing backbones | Linker |
| aminoalkylphosphoramidates | Linker |
| aminoalkylphosphotriesters | Linker |
| boranophosphates | Linker |
| —CH2—O—N(CH3)—CH2— | Linker |
| —CH2—N(CH3)—N(CH3)—CH2— | Linker |
| —CH2—NH—CH2— | Linker |
| chiral phosphonates | Linker |
| chiral phosphorothioates | Linker |
| formacetyl and thioformacetyl backbones | Linker |
| methylene (methylimin) | Linker |
| methylene formacetyl and thioformacetyl backbones | Linker |
| methyleneimino and methylenehydrazino backbones | Linker |
| morpholino linkages | Linker |
| —N(CH3)—CH2—CH2— | Linker |
| oligonucleosides with heteroatom intemucleoside linkage | Linker |
| phosphinates | Linker |
| phosphoramidates | Linker |
| phosphorodithioates | Linker |
| phosphorothioate intemucleoside linkages | Linker |
| phosphorothioatesphosphotriesters | Linker |
| PNA | Linker |
| siloxane backbones | Linker |
| sulfamate backbones | Linker |
| sulfide sulfoxide and sulfone backbones | Linker |
| sulfonate and sulfonamide backbones | Linker |
| thionoalkylphosphonates | Linker |
| thionoalkylphosphotriesters | Linker |
| thionophosphoramidates | Linker |

The polynucleotide can include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present invention may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modified polynucleotides to be used in the present invention do not substantially induce an innate immune response of a cell into which the mRNA is introduced. Features of an induced innate immune response include 1) increased expression of pro-inflammatory cytokines, 2) activation of intracellular PRRs (RIG-I. MDAS, etc., and/or 3) termination or reduction in protein translation.

In certain embodiments, it may be desirable to intracellularly degrade a polynucleotide introduced into the cell. For example, degradation of a polynucleotide may be preferable if precise timing of protein production is desired. Thus, in some embodiments, the invention provides a polynucleotide containing a degradation domain, which is capable of being acted on in a directed manner within a cell.

Any of the regions of the polynucleotides may be chemically modified as taught herein or as taught in International Publication Number WO2013052523 and International Publication Number WO2014093924 the contents of each of which are incorporated herein by reference in its entirety.

Sugar Modifications: The modified nucleosides and nucleotides, which may be incorporated into a modified polynucleotide molecule as described herein, can be modified in the sugar moiety. For example, the 2′ hydroxyl group (OH) can be modified or replaced with a number of different substituents.

The modified nucleosides and nucleotides (e.g., building block molecules), which may be incorporated into a polynucleotide (e.g., RNA or mRNA, as described herein), can be modified on the sugar of the ribonucleic acid. For example, the 2′ hydroxyl group (OH) can be modified or replaced with a number of different substituents. Exemplary substitutions at the 2′-position include, but are not limited to, H, halo, optionally substituted C1-6 alkyl; optionally substituted C1-6 alkoxy; optionally substituted C6-10 aryloxy; optionally substituted C3-8 cycloalkyl; optionally substituted C3-8 cycloalkoxy; optionally substituted C6-10 aryloxy; optionally substituted C6-10 aryl-C1-6 alkoxy, optionally substituted C1-12 (heterocyclyl)oxy; a sugar (e.g., ribose, pentose, or any described herein): a polyethyleneglycol (PEG). - O(CH2CH20)nCH2CH20R, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20): “locked” nucleic acids (LNA) in which the 2′-hydroxyl is connected by a C1-6 alkylene or C1-6 heteroalkylene bridge to the 4′-carbon of the same ribose sugar, where exemplary bridges included methylene, propylene, ether, or amino bridges; aminoalkyl, as defined herein; aminoalkoxy, as defined herein; amino as defined herein; and amino acid, as defined herein.

Generally. RNA includes the sugar group ribose, which is a 5-membered ring having an oxygen. Exemplary, non-limiting modified nucleotides include replacement of the oxygen in ribose (e.g., with S. Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g. R-GNA or S-GNA, where ribose is replaced by glycol units attached to phosphodiester bonds), threose nucleic acid (TNA, where ribose is replace with α-L-threofuranosyl-(3′→2′), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar. Such sugar modifications are taught in International Publication No. WO 2013/052523 and International Publication No. WO 2014/093924 the contents of each of which are incorporated herein by reference in its entirety.

Building blocks, e.g., modified ribonucleotides, and modified ribonucleotides, of polynucleotide molecules useful for preparing the modified polynucleotides for use in the present invention are taught in International Publication Number WO 2013/052523 and International Publication Number WO 2014/093924 the contents of each of which are incorporated herein by reference in its entirety.

### Base Modifications:

The present disclosure provides for modified nucleosides and nucleotides. As described herein “nucleoside” is defined as a compound containing a sugar molecule (e.g., a pentose or ribose) or a derivative thereof in combination with an organic base (e.g., a purine or pyrimidine) or a derivative thereof (also referred to herein as “nucleobase”). As described herein, “nucleotide” is defined as a nucleoside including a phosphate group. The modified nucleotides may be synthesized by any useful method, as described herein (e.g., chemically, enzymatically, or recombinantly to include one or more modified or non-natural nucleosides). The polynucleotides may comprise a region or regions of linked nucleosides. Such regions may have variable backbone linkages. The linkages may be standard phosphoester linkages, in which case the polynucleotides would comprise regions of nucleotides.

The modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil.

The modified nucleosides and nucleotides can include a modified nucleobase. Examples of nucleobases found in RNA include, but are not limited to, adenine, guanine, cytosine, and uracil. Examples of nucleobase found in DNA include, but are not limited to, adenine, guanine, cytosine, and thymine. Such modified nucleobases (including the distinctions between naturally occurring and non-naturally occurring) are taught in International Publication No. WO 2013/052523 and International Publication No. WO 2014/093924 the contents of each of which are incorporated herein by reference in its entirety.

The polynucleotides of the invention can include a combination of modifications to the sugar, the nucleobase, and/or the internucleoside linkage. These combinations can include any one or more modifications described herein.

Examples of modified nucleotides and modified nucleotide combinations are provided below in Tables D and E. These combinations of modified nucleotides can be used to form the modified polynucleotides for use in the present invention. Unless otherwise noted, the modified nucleotides may be completely substituted for the natural nucleotides of the polynucleotides of the invention. As a non-limiting example, the natural nucleotide uridine may be substituted with a modified nucleoside described herein. In another non-limiting example, the natural nucleotide uridine may be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9%) with at least one of the modified nucleoside disclosed herein. Any combination of base/sugar or linker may be incorporated into the modified polynucleotides for use in the present invention and such modifications are taught in International Publication Number WO 2013/052523 and in International Publication Number WO 2014/093924 the contents of each of which are incorporated herein by reference in its entirety.

TABLE D
Combinations

| Modified | |
|---|---|
| Nucleotide | Modified Nucleotide Combination |
| α-thio- | α-thia-cytidine/5-iodo-uridine |
| cytidine | α-thia-cytidine/N1-methyl-pseudouridine |
| | α-thin-cytidine/α-thio-uridine |
| | α-thia-cytidine/5-methyl-uridine |
| | α-thio-cytidine/pseudo-uridine |
| | about 50% of the cytosines are α-thio-cytidine |
| pseudoisocytidine | pseudoisocytidine/5-iodo-uridine |
| | pseudoisocytidine/N1-methyl-pseudouridine |
| | pseudoisocytidine/α-thio-uridine |
| | pseudoisocytidine/5-methyl-uridine |
| | pseudoisocytidine/pseudouridine |
| | about 25% of cytosines are pseudoisocytidine |
| | pseudoisocytidine/about 50% ofuridines are |
| | N1-methyl-pseudouridine and about 50% of |
| | uridine are pseudouridine |
| | pseudoisocytidine/about 25% of uridines are |
| | N1-methyl-pseudouridine and about 25% |
| | ofuridines are pseudouridine |
| pyrrolo- | pyrrolo-cytidine/5-iodo-uridine |
| cytidine | pyrrolo-cytidine/N1-methyl-pseudouridine |
| | pyrrolo-cytidine/α-thio-uridine |
| | pyrrolo-cytidine/5-methyl-uridine |
| | pyrrolo-cytidine/pseudouridine |
| | about 50% of the cytosines are pyrrolo-cytidine |
| 5-methyl- | 5-methyl-cytidine/5-iodo-uridine |
| cytidine | 5-methyl-cytidine/N1-methyl-pseudouridine |
| | 5-methyl-cytidine/α-thio-uridine |
| | 5-methyl-cytidine/5-methyl-uridine |
| | 5-methyl-cytidine/pseudouridine |
| | about 25% of cytosines are 5-methyl-cytidine |
| | about 50% of cytosines are 5-methyl-cytidine |
| | 5-methyl-cytidine/5-methoxy-uridine |
| | 5-methyl-cytidine/5-bromo-uridine |
| | 5-methyl-cytidine/2-thio-uridine |
| | 5-methyl-cytidine/about 50% of uridines are |
| | 2-thio-uridine |
| | about 50% of uridines are 5-methyl-cytidine/about |
| | 50% of uridines are 2-thio-uridine |
| N4-acetyl- | N4-acetyl-cytidine/5-iodo-uridine |
| cytidine | N4-acetyl-cytidine/N1-methyl-pseudouridine |
| | N4-acetyl-cytidine/α-thio-uridine |
| | N4-acetyl-cytidine/5-methyl-uridine |
| | N4-acetvl-cytidine/pseudouridine |
| | about 50% of cytosines are N4-acetyl-cytidine |
| | about 25% of cytosines are N4-acetyl-cytidine |
| | N4-acetyl-cytidine/5-methoxy-uridine |
| | N4-acetyl-cytidine/5-bromo-uridine |
| | N4-acetyl-cytidine/2-thio-uridine |
| | about 50% of cytosines are N4-acetyl-cytidine/about |
| | 50% of uridines are 2-thio-uridine |

TABLE E
Combinations
Combination

1-(2,2,2-Trifluoroethyl)pseudo-UTP
1-Ethyl-pseudo-UTP
1-Methyl-pseudo-U-alpha-thio-TP
1-methyl-pseudouridine TP, ATP, GTP, CTP
1-methyl-pseudo-UTP/5-methyl-CTP/ATP/GTP
1-Propyl-pseudo-UTP
25% 5-Aminoallyl-CTP + 75% CTP/25% 5-Methoxy-UTP + 75% UTP
25% 5-Aminoallyl-CTP + 75% CTP/75% 5-Methoxy-UTP + 25% UTP
25% 5-Bromo-CTP + 75% CTP/25% 5-Methoxy-UTP + 75% UTP
25% 5-Bromo-CTP + 75% CTP/75% 5-Methoxy-UTP + 25% UTP
25% 5-Bromo-CTP + 75% CTP/1-Methyl-pseudo-UTP
25% 5-Carboxy-CTP + 75% CTP/25% 5-Methoxy-UTP + 75% UTP
25% 5-Carboxy-CTP + 75% CTP/75% 5-Methoxy-UTP + 25% UTP
25% 5-Ethyl-CTP + 75% CTP/25% 5-Methoxy-UTP + 75% UTP
25% 5-Ethyl-CTP + 75% CTP/75% 5-Methoxy-UTP + 25% UTP
25% 5-Ethynyl-CTP + 75% CTP/25% 5-Methoxy-UTP + 75% UTP
25% 5-Ethynyl-CTP + 75% CTP/75% 5-Methoxy-UTP + 25% UTP
25% 5-Fluoro-CTP + 75% CTP/25% 5-Methoxy-UTP + 75% UTP
25% 5-Fluoro-CTP + 75% CTP/75% 5-Methoxy-UTP + 25% UTP
25% 5-Formyl-CTP + 75% CTP/25% 5-Methoxy-UTP + 75% UTP
25% 5-Formyl-CTP + 75% CTP/75% 5-Methoxy-UTP + 25% UTP
25% 5-Hydroxymethyl-CTP + 75% CTP/25% 5-Methoxy-UTP + 75% UTP
25% 5-Hydroxymethyl-CTP + 75% CTP/75% 5-Methoxy-UTP + 25% UTP
25% 5-Iodo-CTP + 75% CTP/25% 5-Methoxy-UTP + 75% UTP
25% 5-Iodo-CTP + 75% CTP/75% 5-Methoxy-UTP + 25% UTP
25% 5-Methoxy-CTP + 75% CTP/25% 5-Methoxy-UTP + 75% UTP
25% 5-Methoxy-CTP + 75% CTP/75% 5-Methoxy-UTP + 25% UTP
25% 5-Methyl-CTP + 75% CTP/25% 5-Methoxy-UTP + 75% 1-Methyl-pseudo-UTP
25% 5-Methyl-CTP + 75% CTP/25% 5-Methoxy-UTP + 75% UTP
25% 5-Methyl-CTP + 75% CTP/50% 5-Methoxy-UTP + 50% 1-Methyl-pseudo-UTP
25% 5-Methyl-CTP + 75% CTP/50% 5-Methoxy-UTP + 50% UTP
25% 5-Methyl-CTP + 75% CTP/5-Methoxy-UTP
25% 5-Methyl-CTP + 75% CTP/75% 5-Methoxy-UTP + 25% 1-Methyl-pseudo-UTP
25% 5-Methyl-CTP + 75% CTP/75% 5-Methoxy-UTP + 25% UTP
25% 5-Phenyl-CTP + 75% CTP/25% 5-Methoxy-UTP + 75% UTP
25% 5-Phenyl-CTP + 75% CTP/75% 5-Methoxy-UTP + 25% UTP
25% 5-Trifluoromethyl-CTP + 75% CTP/25% 5-Methoxy-UTP + 75% UTP
25% 5-Trifluoromethyl-CTP + 75% CTP/75% 5-Methoxy-UTP + 25% UTP
25% 5-Trifluoromethyl-CTP + 75% CTP/1-Methyl-pseudo-UTP
25% N4—Ac—CTP + 75% CTP/25% 5-Methoxy-UTP + 75% UTP
25% N4—Ac—CTP + 75% CTP/75% 5-Methoxy-UTP + 25% UTP
25% N4—Bz—CTP + 75% CTP/25% 5-Methoxy-UTP + 75% UTP
25% N4—Bz—CTP + 75% CTP/75% 5-Methoxy-UTP + 25% UTP
25% N4-Methyl-CTP + 75% CTP /25% 5-Methoxy-UTP + 75% UTP
25% N4-Methyl-CTP + 75% CTP/75% 5-Methoxy-UTP + 25% UTP
25% Pseudo-iso-CTP + 75% CTP/25% 5-Methoxy-UTP + 75% UTP
25% Pseudo-iso-CTP + 75% CTP/75% 5-Methoxy-UTP + 25% UTP
25% 5-Bromo-CTP/75% CTP/Pseudo-UTP
25% 5-methoxy-UTP/25% 5-methyl-CTP/ATP/GTP
25% 5-methoxy-UTP/5-methyl-CTP/ATP/GTP
25% 5-methoxy-UTP/75% 5-methyl-CTP/ATP/GTP
25% 5-methoxy-UTP/CTP/ATP/GTP
25% 5-metoxy-UTP/50% 5-methyl-CTP/ATP/GTP
2-Amino-ATP
2-Thio-CTP
2-thio-pseudouridine TP, ATP, GTP, CTP
2-Thio-pseudo-UTP
2-Thio-UTP
3-Methyl-CTP
3-Methyl-pseudo-UTP
4-Thio-UTP
50% 5-Bromo-CTP + 50% CTP/1-Methyl-pseudo-UTP
50% 5-Hydroxymethyl-CTP + 50% CTP/1-Methyl-pseudo-UTP
50% 5-methoxy-UTP/5-methyl-CTP/ATP/GTP
50% 5-Methyl-CTP + 50% CTP/25% 5-Methoxy-UTP + 75% 1-Methyl-pseudo-UTP
50% 5-Methyl-CTP + 50% CTP/25% 5-Methoxy-UTP + 75% UTP
50% 5-Methyl-CTP + 50% CTP/50% 5-Methoxy-UTP + 50% 1-Methyl-pseudo-UTP
50% 5-Methyl-CTP + 50% CTP/50% 5-Methoxy-UTP + 50% UTP
50% 5-Methyl-CTP + 50% CTP/5-Methoxy-UTP
50% 5-Methyl-CTP + 50% CTP/75% 5-Methoxy-UTP + 25% 1-Methyl-pseudo-UTP
50% 5-Methyl-CTP + 50% CTP/75% 5-Methoxy-UTP + 25% UTP
50% 5-Trifluoromethyl-CTP + 50% CTP/1-Methyl-pseudo-UTP
50% 5-Bromo-CTP/50% CTP/Pseudo-UTP
50% 5-methoxy-UTP/50% 5-methyl-CTP/ATP/GTP
50% 5-methoxy-UTP/75% 5-methyl-CTP/ATP/GTP
5-Aminoallyl-CTP
5-Aminoallyl-CTP/5-Methoxy-UTP
5-Aminoallyl-UTP
5-Bromo-CTP
5-Bromo-CTP/5-Methoxy-UTP
5-Bromo-CTP/1-Methyl-pseudo-UTP
5-Bromo-CTP/Pseudo-UTP
5-bromocytidine TP, ATP, GTP, UTP
5-Bromo-UTP
5-Carboxy-CTP/5-Methoxy-UTP
5-Ethyl-CTP/5-Methoxy-UTP
5-Ethynyl-CTP/5-Methoxy-UTP
5-Fluoro-CTP/5-Methoxy-UTP
5-Formyl-CTP/5-Methoxy-UTP
5-Hydroxy-methyl-CTP/5-Methoxy-UTP
5-Hydroxymethyl-CTP
5-Hydroxymethyl-CTP/1-Methyl-pseudo-UTP
5-Hydroxymethyl-CTP/5-Methoxy-UTP
5-hydroxymethyl-cytidine TP, ATP, GTP, UTP
5-Iodo-CTP/5-Methoxy-UTP
5-Me—CTP/5-Methoxy-UTP
5-Methoxy carbonyl methyl-UTP
5-Methoxy-CTP/5-Methoxy-UTP
5-methoxy-uridine TP, ATP, GTP, UTP
5-methoxy-UTP
5-Methoxy-UTP
5-Methoxy-UTP/NG-Isopentenyl-ATP
5-methoxy-UTP/25% 5-methyl-CTP/ATP/GTP
5-methoxy-UTP/5-methyl-CTP/ATP/GTP
5-methoxy-UTP/75% 5-methyl-CTP/ATP/GTP
5-methoxy-UTP/CTP/ATP/GTP
5-Methyl-2-thio-UTP
5-Methylaminomethyl-UTP
5-Methyl-CTP/5-Methoxy-UTP
5-Methyl-CTP/5-Methoxy-UTP(cap O)
5-Methyl-CTP/5-Methoxy-UTP(No cap)
5-Methyl-CTP/25% 5-Methoxy-UTP + 75% 1-Methyl-pseudo-UTP
5-Methyl-CTP/25% 5-Methoxy-UTP + 75% UTP
5-Methyl-CTP/50% 5-Methoxy-UTP + 50% 1-Methyl-pseudo-UTP
5-Methyl-CTP/50% 5-Methoxy-UTP + 50% UTP
5-Methyl-CTP/5-Methoxy-UTP/N6—Me—ATP
5-Methyl-CTP/75% 5-Methoxy-UTP + 25% 1-Methyl-pseudo-UTP
5-Methyl-CTP/75% 5-Methoxy-UTP + 25% UTP
5-Phenyl-CTP/5-Methoxy-UTP
5-Trifluoro-methyl-CTP/5-Methoxy-UTP
5-Trifluoromethyl-CTP
5-Trifluoromethyl-CTP/5-Methoxy-UTP
5-Trifluoromethyl-CTP/1-Methyl-pseudo-UTP
5-Trifluoromethyl-CTP/Pseudo-UTP
5-Trifluoromethyl-UTP
5-trifluromethylcytidine TP, ATP, GTP, UTP
75% 5-Aminoallyl-CTP + 25% CTP/25% 5-Methoxy-UTP + 75% UTP
75% 5-Aminoallyl-CTP + 25% CTP/75% 5-Methoxy-UTP + 25% UTP
75% 5-Bromo-CTP + 25% CTP/25% 5-Methoxy-UTP + 75% UTP
75% 5-Bromo-CTP + 25% CTP/75% 5-Methoxy-UTP + 25% UTP
75% 5-Carboxy-CTP + 25% CTP/25% 5-Methoxy-UTP + 75% UTP
75% 5-Carboxy-CTP + 25% CTP/75% 5-Methoxy-UTP + 25% UTP
75% 5-Ethyl-CTP + 25% CTP/25% 5-Methoxy-UTP + 75% UTP
75% 5-Ethyl-CTP + 25% CTP/75% 5-Methoxy-UTP + 25% UTP
75% 5-Ethynyl-CTP + 25% CTP/25% 5-Methoxy-UTP + 75% UTP
75% 5-Ethynyl-CTP + 25% CTP/75% 5-Methoxy-UTP + 25% UTP
75% 5-Fluoro-CTP + 25% CTP/25% 5-Methoxy-UTP + 75% UTP
75% 5-Fluoro-CTP + 25% CTP/75% 5-Methoxy-UTP + 25% UTP
75% 5-Formyl-CTP + 25% CTP/25% 5-Methoxy-UTP + 75% UTP
75% 5-Formyl-CTP + 25% CTP/75% 5-Methoxy-UTP + 25% UTP
75% 5-Hydroxymethyl-CTP + 25% CTP/25% 5-Methoxy-UTP + 75% UTP
75% 5-Hydroxymethyl-CTP + 25% CTP/75% 5-Methoxy-UTP + 25% UTP
75% 5-Iodo-CTP + 25% CTP/25% 5-Methoxy-UTP + 75% UTP
75% 5-Iodo-CTP + 25% CTP/75% 5-Methoxy-UTP + 25% UTP
75% 5-Methoxy-CTP + 25% CTP/25% 5-Methoxy-UTP + 75% UTP
75% 5-Methoxy-CTP + 25% CTP/75% 5-Methoxy-UTP + 25% UTP
75% 5-methoxy-UTP/5-methyl-CTP/ATP/GTP
75% 5-Methyl-CTP + 25% CTP/25% 5-Methoxy-UTP + 75% 1-Methyl-pseudo-UTP
75% 5-Methyl-CTP + 25% CTP/25% 5-Methoxy-UTP + 75% UTP
75% 5-Methyl-CTP + 25% CTP/50% 5-Methoxy-UTP + 50% 1-Methyl-pseudo-UTP
75% 5-Methyl-CTP + 25% CTP/50% 5-Methoxy-UTP + 50% UTP
75% 5-Methyl-CTP + 25% CTP/5-Methoxy-UTP
75% 5-Methyl-CTP + 25% CTP/75% 5-Methoxy-UTP + 25% 1-Methyl-pseudo-UTP
75% 5-Methyl-CTP + 25% CTP/75% 5-Methoxy-UTP + 25% UTP
75% 5-Phenyl-CTP + 25% CTP/25% 5-Methoxy-UTP + 75% UTP
75% 5-Phenyl-CTP + 25% CTP/75% 5-Methoxy-UTP + 25% UTP
75% 5-Trifluoromethyl-CTP + 25% CTP/25% 5-Methoxy-UTP + 75% UTP
75% 5-Trifluoromethyl-CTP + 25% CTP/75% 5-Methoxy-UTP + 25% UTP
75% 5-Trifluoromethyl-CTP + 25% CTP/1-Methyl-pseudo-UTP
75% N4-Ac-CTP + 25% CTP/25% 5-Methoxy-UTP + 75% UTP
75% N4-Ac-CTP + 25% CTP/75% 5-Methoxy-UTP + 25% UTP
75% N4-Bz-CTP + 25% CTP/25% 5-Methoxy-UTP + 75% UTP
75% N4-Bz-CTP + 25% CTP/75% 5-Methoxy-UTP + 25% UTP
75% N4-Methyl-CTP + 25% CTP/25% 5-Methoxy-UTP + 75% UTP
75% N4-Methyl-CTP + 25% CTP/75% 5-Methoxy-UTP + 25% UTP
75% Pseudo-iso-CTP + 25% CTP/25% 5-Methoxy-UTP + 75% UTP
75% Pseudo-iso-CTP + 25% CTP/75% 5-Methoxy-UTP + 25% UTP
75% 5-Bromo-CTP/25% CTP/1-Methyl-pseudo-UTP
75% 5-Bromo-CTP/25% CTP/Pseudo-UTP
75% 5-methoxy-UTP/25% 5-methyl-CTP/ATP/GTP
75% 5-methoxy-UTP/50% 5-methyl-CTP/ATP/GTP
75% 5-methoxy-UTP/75% 5-methyl-CTP/ATP/GTP
75% 5-methoxy-UTP/CTP/ATP/GTP
8-Aza-ATP
Alpha-thio-CTP
CTP/25% 5-Methoxy-UTP + 75% 1-Methyl-pseudo-UTP
CTP/25% 5-Methoxy-UTP + 75% UTP
CTP/50% 5-Methoxy-UTP + 50% 1-Methyl-pseudo-UTP
CTP/50% 5-Methoxy-UTP + 50% UTP CTP/5-Methoxy-UTP
CTP/5-Methoxy-UTP(cap O)
CTP/5-Methoxy-UTP(No cap)
CTP/75% 5-Methoxy-UTP + 25% 1-Methyl-pseudo-UTP
CTP/75% 5-Methoxy-UTP + 25% UTP
CTP/UTP (No cap)
N1—Me—GTP
N4—Ac—CTP
N4Ac—CTP/1-Methyl-pseudo-UTP
N4Ac—CTP/5-Methoxy-UTP
N4-acetyl-cytidine TP, ATP, GTP, UTP
N4—Bz—CTP/5-Methoxy-UTP
N4-methyl CTP
N4-Methyl-CTP/5-Methoxy-UTP
Pseudo-iso-CTP/5-Methoxy-UTP
PseudoU-alpha-thio-TP
pseudouridine TP, ATP, GTP, CTP
pseudo-UTP/5-methyl-CTP/ATP/GTP
UTP-5-oxyacetic acid Me ester
Xanthosine

According to the invention, the modified polynucleotides for use in the present invention may be synthesized to comprise the combinations or single modifications of Tables D or E.

Where a single modification is listed, the listed nucleoside or nucleotide represents 100 percent of that A. U. G or C nucleotide or nucleoside having been modified. Where percentages are listed, these represent the percentage of that particular A, U. G or C nucleobase triphosphate of the total amount of A. U. G, or C triphosphate present. For example, the combination: 25% 5-Aminoallyl-CTP+75% CTP/ 25% 5-Methoxy-UTP+75% UTP refers to a polynucleotide where 25% of the cytosine triphosphates are 5-Aminoallyl-CTP while 75% of the cytosines are CTP; whereas 25% of the uracils are 5-methoxy UTP while 75% of the uracils are UTP. Where no modified UTP is listed then the naturally occurring ATP, UTP, GTP and/or CTP is used at 100% of the sites of those nucleotides found in the polynucleotide. In this example all of the GTP and ATP nucleotides are left unmodified.

In a preferred embodiment, the modifications are selected from base modifications, which are preferably selected from pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl)adenosine, N6-glycinylcarbamoyladenosine. N6-threonylcarbamoyladenosine, 2-methylthio-N6-threonylcarbamoyladenosine, NG, N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2.N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

Lipid modification: According to a further embodiment, the modified polynucleotide as defined herein can contain a lipid modification. Such a lipid-modified polynucleotide typically comprises a polynucleotide as defined herein. Such a lipid-modified polynucleotide as defined herein typically further comprises at least one linker covalently linked with that polynucleotide, and at least one lipid covalently linked with the respective linker. Alternatively, the lipid-modified polynucleotide comprises at least one polynucleotide as defined herein and at least one (bifunctional) lipid covalently linked (without a linker) with that polynucleotide. According to a third alternative, the lipid-modified polynucleotide comprises an polynucleotide as defined herein, at least one linker covalently linked with that polynucleotide, and at least one lipid covalently linked with the respective linker, and also at least one (bifunctional) lipid covalently linked (without a linker) with that polynucleotide molecule. In this context, it is particularly preferred that the lipid modification is present at the terminal ends of a linear polynucleotide sequence.

Modification of the 5-end of the modified polynucleotide: According to another preferred embodiment of the invention, the modified polynucleotide as defined herein can be modified by the addition of a so-called “5′ CAP” structure.

A 5′-cap is an entity, typically a modified nucleotide entity, which generally “caps” the 5′-end of polynucleotide. A 5′-cap may typically be formed by a modified nucleotide, particularly by a derivative of a guanine nucleotide. Preferably, the 5′-cap is linked to the 5′-terminus via a 5′-5′-triphosphate linkage. A 5′-cap may be methylated, e.g. m7GpppN, wherein N is the terminal 5′ nucleotide of the nucleic acid carrying the 5′-cap, typically the 5′-end of a polynucleotide, m7GpppN is the 5′-CAP structure which naturally occurs in mRNA transcribed by polymerase II and is therefore not considered as modification comprised in the modified polynucleotide according to the invention. This means that the modified polynucleotide to be used according to the present invention may comprise a m7GpppN as 5′-CAP, but additionally the modified polynucleotide comprises at least one further modification as defined herein. Further examples of 5′cap structures include glyceryl, inverted deoxy abasic residue (moiety). 4′, 5′-methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranos 1 nucleotide, acyclic 3′,4′-seco nucleotide, acyclic 3,4-dihydroxybutyl nucleotide, acyclic 3,5-dihydroxypentyl nucleotide, 3′-3′-inverted nucleotide moiety, 3′-3′-inverted abasic moiety, 3′-2′-inverted nucleotide moiety, 3′-2′-inverted abasic moiety. 1,4-butanediol phosphate, 3′-phosphoramidate, hexylphosphate, aminohexyl phosphate. 3′-phosphate, 3′phosphorothioate, phosphorodithioate, or bridging or non-bridging methylphosphonate moiety. These modified 5′-CAP structures are regarded as at least one modification comprised in the modified polynucleotide for use according to the present invention.

Particularly preferred modified 5′-CAP structures are CAP1 (methylation of the ribose of the adjacent nucleotide of m7G). CAP2 (methylation of the ribose of the 2nd nucleotide downstream of the m7G), CAP3 (methylation of the ribose of the 3rd nucleotide downstream of the m7G), CAP4 (methylation of the ribose of the 4th nucleotide downstream of the m7G). ARCA (anti-reverse CAP analogue, modified ARCA (e.g. phosphothioate modified ARCA), inosine, NI-methyl-guanosine, 2′-fluoro-guanosine, 7-deaza-guanosine, 8-oxo-guanosine, 2-amino-guanosine, LNA-guanosine, and 2-azido-guanosine.

Codon optimization: A further preferred modification of the coding region encoding at least one peptide or protein of the polynucleotide as defined herein is based on the finding that the translation efficiency is also determined by a different frequency in the occurrence of tRNAs in cells. Thus, if so-called “rare codons” are present in the coding region of the wild type RNA sequence to an increased extent, the mRNA is translated to a significantly poorer degree than in the case where codons coding for relatively “frequent” tRNAs are present.

In this context, the coding region of the polynucleotide to be used in the present invention is preferably modified compared to the corresponding wild-type coding region such that at least one codon of the wild-type sequence, which codes for a tRNA which is relatively rare in the cell, is exchanged for a codon, which codes for a tRNA which is relatively frequent in the cell and carries the same amino acid as the relatively rare tRNA. By this modification, the coding region of the polynucleotide as defined herein, is modified such that codons, for which frequently occurring tRNAs are available, are inserted. In other words, according to the invention, by this modification all codons of the wild type coding region, which code for a tRNA which is relatively rare in the cell, can in each case be exchanged for a codon, which codes for a tRNA which is relatively frequent in the cell and which, in each case, carries the same amino acid as the relatively rare tRNA.

Which tRNAs occur relatively frequently in the cell and which, in contrast, occur relatively rarely is known to a person skilled in the art; cf. e.g. Akashi, Curr. Opin. Genet. Dev. 2001, 11 (6): 660-666. The codons which use for the particular amino acid the tRNA which occurs most frequently in the (human) cell, are particularly preferred.

In a preferred embodiment, the polynucleotide as defined herein may include at least two stop codons before the 3′ untranslated region (UTR). In a more preferred embodiment, the polynucleotide comprises two stop codons. In an even more preferred embodiment, the polynucleotide comprises a first stop codon “TGA” and a second stop codon selected from the group consisting of “TAA.” “TGA” and “TAG.”

Modification of the 3′-end of the modified polynucleotide: Furthermore, in the modified polynucleotide, the region 3′ of the coding region may be modified, e.g. by insertion or addition of stretches of multiple repeats of adenine or cytosine residues. In a preferred embodiment of the invention, the 3′ end of a polynucleotide molecule is modified by addition of a series of adenine nucleotides (“poly(A) tail”).

The length of the poly(A) tail has a major impact on translation efficiency of the respective construct. To be translated efficiently, the poly(A) tail of exogenously delivered mRNAs should consist of at least 20 adenosine residues. Moreover, it has been described that mRNA expression positively correlates with poly(A) tail length (reviewed in Tavernier et al., J Control Release. 2011 Mar. 30; 1 50(3): 238-47. PMID: 20970469). In this context Holtkamp et al, showed that a poly(A) tail measuring 120 nucleotides compared with a shorter one, enhanced mRNA stability and translational efficiency (Holtkamp et al., Blood. 2006 Dec. 15:108(13):4009-1 7. PMID: 16940422). In one embodiment of the invention, the modified polynucleotide comprises (optionally in combination with other modifications) a poly(A) tail, wherein the poly(A) tail comprises at least 30, preferably more than 50, more preferably more than 100, even more preferably more than 200 adenine nucleotides. Most preferably the polynucleotide comprises a poly(A) tail consisting of 64 adenine nucleotides.

In specific embodiments a poly(A) tail is only considered as at least one modification comprised in the modified polynucleotide according to the present invention if the poly(A) tail is longer than 30 adenosines, preferably longer than 50 adenosines, more preferably longer than 100 adenosines and even more preferably longer than 200 adenosines.

In a preferred embodiment, the polynucleotide may comprise a poly (A)-G quartet as a 3′ tailing sequence. The G-quartet is a cyclic hydrogen bonded array of four guanine nucleotides that can be formed by G-rich sequences. In a particular preferred embodiment, the 3′ tailing sequence is selected from the group consisting of a poly(A) tail of approximately 160 nucleotides and a poly(A)-G quartet.

UTR modification: Preferably, the modified polynucleotide according to the invention has at least one modified 5′ and/or 3′ UTR sequence (UTR modification). These modifications in the 5′ and/or 3′ untranslated regions (UTR) may have the effect of increasing the half-life of the polynucleotide in the cytosol or may increase the translational efficiency and may thus enhance the expression of the encoded protein or peptide. These UTR sequences can have 100% sequence identity to naturally occurring sequences which occur in viruses, bacteria and eukaryotes, but can also be partly or completely synthetic. The untranslated sequences (UTR) of the (alpha-) or beta globin gene, e.g. from Homo sapiens or Xenopus laevis may be mentioned as an example of stabilizing sequences which can be used for a stabilized polynucleotide. Another example of a stabilizing sequence has the general formula (C/U)CCANxCCC(U/A)PyxUC(C/U)CC which is contained in the 3′UTR of the very stable RNA which codes for (alpha-)globin, type(I)-collagen, 1 5-lipoxygenase or for tyrosine hydroxylase (cf. Holcik et al., Proc. Natl. Acad. Sci. USA 1997, 94: 2410 to 2414). Such introduced UTR sequences can, of course, be used individually or in combination with one another and also in combination with other sequence modifications known to a person skilled in the art.

Preferably, decay signals, such as Adenylate Uridylate Rich Elements (AREs) are removed from the 3′ untranslated regions (3′ UTRs) of a polynucleotide for use in the invention. In a preferred embodiment, a combination of two or more of the modifications described above are present in the modified polynucleotide according to the invention.

In a preferred embodiment of the present invention, the modified polynucleotide comprises at least two modifications. In a more preferred embodiment, the modifications are located on one or more of a nucleoside and/or the backbone of the modified polynucleotide. In an even more preferred embodiment, the modifications are located on both a nucleoside and a backbone linkage.

In another preferred embodiment, the at least two modifications are located on the backbone linkage. In a more preferred embodiment, the at least two modifications of the backbone linkage comprise replacing of one or more oxygen atoms. In another preferred embodiment, the at least two modifications of the backbone linkage comprise replacing at least one phosphodiester linkage with a phosphorothioate linkage.

In another preferred embodiment, the at least two modifications are located on a nucleoside.

In yet another preferred embodiment, the at least two modifications are located on a sugar of the nucleoside.

In still another preferred embodiment, the at least two modifications are located on a nucleobase. In a particular preferred embodiment, the modifications on the nucleobase are selected from the group consisting of cytosine, guanine, adenine, thymine and uracil.

### Example

The following example is intended to illustrate the invention in a further way. It is merely illustrative and not intended to limit the subject matter of the invention.

### 1. Synthesis of Polynucleotide

The person skilled in the art is aware of methods to produce a polynucleotide for use in accordance with the present invention. The polynucleotide may be prepared according to any available technique including, but not limited to chemical synthesis, enzymatic synthesis (in vitro transcription) or enzymatic or chemical cleavage of a longer precursor.

In the following synthesis of RNA by using RNA in vitro transcription is described in more detail:

### a) Providing Template DNA

A DNA template for RNA in vitro transcription may be obtained by cloning of a nucleic acid, in particular cDNA corresponding to the respective RNA to be transcribed in vitro, and introducing it into an appropriate vector for in vitro transcription, for example into plasmid DNA. The cDNA may be obtained by reverse transcription of mRNA or chemical synthesis. Moreover, the DNA template for in vitro RNA synthesis may also be obtained by gene synthesis.

The template DNA comprises all elements necessary for RNA in vitro transcription, particularly a promoter element for binding of a DNA dependent RNA polymerase as e.g. T3, T7 and SPG RNA polymerases 5′ of the DNA sequence encoding the target RNA sequence. Further, the template DNA may comprise primer binding sites 5′ and/or 3′ of the DNA sequence encoding the target RNA sequence to determine the identity of the DNA sequence encoding the target RNA sequence e.g. by PCR or DNA sequencing.

### b) In Vitro Transcription

Methods for in vitro transcription are known in the art (Geall et al. (2013) Semin. Immunol. 25(2): 152-159; Brunelle et al. (2013) Methods Enzymol. 530:101-14).

Reagents used in said method typically include:
1) a linearized DNA template with a promoter sequence that has a high binding affinity for its respective RNA polymerase such as bacteriophage-encoded RNA polymerases;
2) ribonucleoside triphosphates (NTPs) for the four bases (adenine, cytosine, guanine and uracil);
3) optionally a cap analog as defined below (e.g. m7G(5′)ppp(5′)G (m7G));
4) a DNA-dependent RNA polymerase capable of binding to the promoter sequence within the linearized DNA template (e.g. 17, T3 or SPG RNA polymerase);
5) optionally a ribonuclease (RNase) inhibitor to inactivate any contaminating RNase;
6) optionally a pyrophosphatase to degrade pyrophosphate, which may inhibit transcription;
7) MgCl2, which supplies Mg2+ ions as a co-factor for the polymerase;
8) a buffer to maintain a suitable pH value, which can also contain antioxidants (e.g. DTT), and/or polyamines such as spermidine at optimal concentrations.

If only one template DNA is present in the in vitro transcription reaction, the sample comprising in vitro transcribed RNA will only comprise one RNA species. If two or more different template DNAs are present in the in vitro transcription reaction, the sample comprising in vitro transcribed RNA will comprise two or more different RNA species.

### c) Purification

After in vitro transcription, the desired RNA is purified. Preferably, the RNA is purified by a HPLC procedure as described in WO 2008/077592 A1. Ideally, the RNA has a higher purity and/or integrity after purification than the starting material. The purity may be determined by methods commonly known to the skilled person, e.g. by gas chromatography, quantitative PCR, analytical HPLC or gel electrophoresis.

Table 1: shows characteristic features of each peptide or protein of the invention, whereby each peptide or protein is represented by the above defined formula “c1(Peptide or protein or gene) c2(NCBI RefSeq ID) c3(NCBI RefSeq description) c4(Protein SEQ ID NO) c5(DNA SEQ ID NOs) c6(RNA SEQ ID NOs) c7(Related disease, disorder or condition)”- for feature c7 specific reference is made to Table C, i.e. the Abbreviation Dictionary for the feature “Related disease, disorder or condition” (corresponding to feature “c7”), whereby each Related disease, disorder or condition is depicted with a specific abbreviation and its written form as described herein (cf, detailed explanation of Table 1 and its corresponding formula above). Each of the recited sequences are also provided in the sequence listing of the priority document European Application No. 16001039.3, filed May 4, 2016, the entire contents of which are hereby incorporated by reference.

#c1(A1BG) c2(NP_570602) c3(alpha-1B-glycoprotein precursor) c4(234076) c5(234077, 234078, 234079, 234080, 234081, 234082) c6( ) c7(b, d, c, e, h, g, i, f); #c1(A1CF) c2(NP_001185747) c3(APOBEC1 complementation factor isoform 1) c4(76183) c5(76184, 76185, 76186, 76187, 76188, 76189, 76190, 76191, 76192, 76193, 76194, 76195, 76196, 76197, 76198, 76199) c6( ) c7(x, y, l, A, c, s, u, w, z, r, h, n, v, j, m, k, g, t, q, o, p); #c1(A1CF) c2(NP_001185749) c3(APOBEC1 complementation factor isoform 3) c4(76200, 76201) c5(76202, 76203, 76204, 76205, 76206, 76207, 76208, 76209, 76210, 76211, 76212, 76213) c6( ) c7(x, y, l, A, c, s, u, w, z, r, h, n, v, j, m, k, g, t, q, o, p); #c1(A1CF) c2(XP_005269777) c3(APOBEC1 complementation factor isoform X1) c4(282807, 282808, 282809) c5(282810, 282811, 282812, 282813, 282814, 282815, 282816, 282817, 282818, 282819, 282820, 282821, 282822, 282823, 282824, 282825) c6( ) c7(x, y, l, A, c, s, u, w, z, r, h, n, v, j, m, k, g, t, q, o, p); #c1(A1CF) c2(XP_011538031) c3(APOBEC1 complementation factor isoform X2) c4(335589) c5(335590, 335591, 335592, 335593, 335594, 335595) c6( ) c7(x, y, l, A, c, s, u, w, z, r, h, n, v, j, m, k, g, t, q, o, p); #c1(A1CF) c2(XP_011538032) c3(APOBEC1 complementation factor isoform X3) c4( ) c5(335596) c6( ) c7(x, y, l, A, c, s, u, w, z, r, h, n, v, j, m, k, g, t, q, o, p); #c1(AZM) c2(NP_000005) c3(alpha-2-macroglobulin precursor) c4(1, 2, 3) c5(4, 5, 6, 7, 8, 9, 10, 11, 12, 13, 14, 15, 16, 17, 18, 19, 20, 21) c6( ) c7(P, G, T, am, aw, al, l, O, aj, ah, C, V, ai, J, ad, av, F, M, ab, K, an, H, ae, af, N, aa, ak, Y, ap, aq, S, as, Z, ar, D, W, ac, ag, au, X, U, at, E, R, Q, y, B, ao, L); #c1(A2MLI) c2(NP_653271) c3(alpha-2-macroglobulin-like protein 1 isoform 1 precursor) c4(239450, 239451, 239452) c5(239453, 239454, 239455, 239456, 239457, 239458, 239459, 239460, 239461, 239462, 239463, 239464, 239465, 239466, 239467, 239468, 239469, 239470) c6( ) c7(ax, af, ay); #c1(A4GNT) c2(NP_057245) c3(alpha-1,4-N-acetylglucosaminyltransferase) c4(189701) c5(189702, 189703, 189704, 189705, 189706, 189707) c6( ) c7(T, aC, aE, aD, az, aA, n, aF, aB); #c1(AAAS) c2(NP_001166937) c3(aladin isoform 2) c4(68679) c5(68680, 68681, 68682, 68683, 68684, 68685) c6( ) c7(T, aG, aO, e, aZ, l, aj, v, j, bh, C, bd, aY, aR, J, aV, aX, aN, aT, aU, h, bj, aQ, K, bl, aH, af, bc, ak, bf, bn, aJ, Y, aM, aW, aP, bg, aq, bp, ba, bb, bo, U, bm, bk, aL, aS, be, bi, al, B); #c1(AAAS) c2(NP_056480) c3(aladin isoform 1) c4(187708) c5(187709, 187710, 187711, 187712, 187713, 187714) c60) c7(T, aG, aO, e, aZ, l, aj, v, j, bh, C, bd, aY, aR, J, aV, aX, aN, aT, aU, h, bj, aQ, K, bl, aH, af, bc, ak, bf, bn, aJ, Y, aM, aW, aP, bg, aq, bp, ba, bb, bo, U, bm, bk, aL, aS, be, bi, al, B); #c1(AAAS) c2(XP_006719682) c3(aladin isoform X6) c4( ) c5(292882) c6( ) c7(T, aG, aO, e, aZ, l, aj, v, j, bh, C, bd, aY, aR, J, aV, aX, aN, aT, aU, h, bj, aQ, K, bl, aH, af, bc, ak, bf, bn, aJ, Y, aM, aW, aP, bg, aq, bp, ba, bb, bo, U, bm, bk, aL, aS, be, bi, al, B); #c1(AACS) c2(NP_076417) c3(acetoacetyl-CoA synthetase) c4(214/05) c5(214106, 214107, 214108, 214109, 214110, 214111) c6( ) c7(v, bq); #c1(AACS) c2(XP_005253667) c3(acetoacetyl-CoA synthetase isoform X2) c4(272615) c5(272616, 272617, 272618, 272619, 272620, 272621, 272622) c6( ) c7(v, bq); #c1(AACS) c2(XP_011536994) c3(acetoacetyl-CoA synthetase isoform X3) c4(334738) c5(334739, 334740, 334741, 334742, 334743) c6( ) c7(v, bq); #c1(AADAC) c2(NP_001077) c3(arylacetamide deacetylase) c4(37900) c5( ) c6( ) c7(bv, d, aW, aT, bs, bw, m, bu, t, bt, br); #c1(AAGAB) c2(NP_078942) c3(alpha- and gamma-adaptin-binding protein p34 isoform 1) c4(216202) c5(216203, 216204, 216205, 216206, 216207, 216208) c6( ) c7(bF, bE, by, bx, bB, bC, bD, bz, bG, bA); #c1(AAKI) c2(NP_055726) c3(AP2-associated protein kinase 1) c4(183130, 183131) c5(183132, 183133, 183134, 183135, 183136, 183137, 183138, 183139, 183140, 183141, 183142, 183143, 183144) c6( ) c7(bH, C, an); #c1(AANAT) c2(NP_001160051) c3(serotonin N-acetyltransferase isoform 1) c4(65057) c5(65058, 65059, 65060, 65061, 65062, 65063) c6( ) c7(bL, bM, bP, bl, aW, Y, bO, aT, r, bQ, bk, bq, Z, q, bN, bJ); #c1(AANAT) c2(XP_011522723) c3(serotonin N-acetyltransferase isoform X2) c4(317253) c5( ) c6( ) c7(bL, bM, bP, bl, aW, Y, bo, aT, r, bQ, bk, bq, Z, q, bN, bJ); #c1(AANAT) c2(XP_011522724) c3(serotonin N-acetyltransferase isoform X3) c4( ) c5(317254) c6( ) c7(bL, bM, bP, bl, aW, Y, bO, aT, r, bQ, bK, bq, Z, q, bN, bJ); #c1(AANAT) c2(XP_011522725) c3(serotonin N-acetyltransferase isoform X1) c4( ) c5(317255, 317256, 317257, 317258, 317259) c6( ) c7(bL, bM, bP, bl, aW, Y, b, aT, r, bQ, bk, bq, Z, q, bN, bJ); #c1(AARD) c2(NP_001020528) c3(alanine and arginine-rich domain-containing protein) c4(28835) c5(28836, 28837, 28838, 28839, 28840, 28841) c6( ) c7(bM); #c1(AARS) c2(NP_001596) c3(alanine-tRNA ligase, cytoplasmic) c4(119003) c5(119004, 119005, 119006, 119007, 119008, 119009) c6( ) c7(bT, bR, bW, bS, bX, bU, bV, p); #c1(AARS2) c2(NP_065796) c3(alanine-tRNA ligase, mitochondrial) c4(205487) c5(205488, 205489, 205490, 205491, 205492, 205493) c6( ) c7(cc, bU, bY, cb, bW, aw, bZ, ca, bV, p); #c1(AASDH) c2(NP_001273597) c3 (acyl-CoA synthetase family member 4 isoform 2) c4(105405, 105406) c5( ) c6( ) c7(g); #c1(AASDH) c2(NP_001273599) c3(acyl-CoA synthetase family member 4 isoform 4) c4( ) c5(105407, 105408, 105409, 105410, 105411, 105412) c6( ) c7(g); #c1(AASDH) c2(NP_001273600) c3(acyl-CoA synthetase family member 4 isoform 5) c4(105413) c5(105414, 105415, 105416, 105417, 105418, 105419) c6( ) c7(g); #c1(AASDH) c2(NP_001273601) c3(acyl-CoA synthetase family member 4 isoform 6) c4(105420) c5(105421, 105422, 105423, 105424, 105425, 105426) c6( ) c7(g); #c1(AASDH) c2(XP_005265778) c3(acyl-CoA synthetase family member 4 isoform X1) c4(279718) c5(279719, 279720, 279721, 279722, 279723, 279724, 279725, 279726, 279727, 279728, 279729, 279730, 279731) c6( ) c7(g); #c1(AASDH) c2(XP_005265784) c3(acyl-CoA synthetase family member 4 isoform X5) c4( ) c5(279732) c6( ) c7(g); #c1(AASDHPPT) c2(NP_056238) c3(L-aminoadipate-semialdehyde dehydrogenase-phosphopantetheinyl transferase) c4(186653) c5(186654, 186655, 186656, 186657, 186658, 186659) c6( ) c7(h, cd, ac); #c1(AASS) c2(NP_005754) c3(alpha-aminoadipic semialdehyde synthase, mitochondrial) c4(159041) c5(159042, 159043, 159044, 159045, 159046, 159047, 159048, 159049, 159050, 159051) c6( ) c7(ce, cf, cg); #c1(AATF) c2(NP_036270) c3(protein AATF) c4(172739) c5(172740, 172741, 172742, 172743, 172744, 172745, 172746) c6( ) c7(ck, co, T, bE, aW, cj, aT, cm, v, ci, cl, ch, cn); #c1(AATK) c2(NP_001073864) c3(serine/threonine-protein kinase LMTKI isoform 1) c4(36168) c5(36169, 36170, 36/71, 36172, 36173, 36174) c6( ) c7(cd, Y, cq, cr, cp); #c1(AATK) c2(XP_005257912) c3(serine/threonine-protein kinase LMTKI isoform X7) c4(275417) c5(275418, 275419, 275420, 275421, 275422, 275423) c6( ) c7(cd, Y, cq, cr, cp); #c1(AATK) c2(XP_006722259) c3(serine/threonine-protein kinase LMTKI isoform X2) c4(294670) c5( ) c6( ) c7(cd, Y, cq, cr, cp); #c1(AATK) c2(XP_011523811) c3(serine/threonine-protein kinase LMTKI isoform X1) c4( ) c5(318349, 318350, 318351, 318352, 318353, 318354) c6( ) c7(cd, Y, cq, cr, cp); #c1(ABAT) c2(XP_011520703) c3(4-aminobutyrate aminotransferase, mitochondrial isoform X1) c4(315740) c5(315741, 315742, 315743, 315744, 315745, 315746, 315747, 315748, 315749, 315750, 315751, 315752, 315753, 315754) c6( ) c7(cw, cF, cs, cA, cE, cC, cz, cD, cy, cu, ct, bq, cx, cB, bN, cv); #c1(ABCA1) c2(NP_005493) c3(ATP-binding cassette sub-family A member 1) c4(156773, 156774) c5(156775, 156776, 156777, 156778, 156779, 156780, 156781, 156782, 156783, 156784, 156785, 156786) c6( ) c7(bM, T, ef, ea, e, eb, bB, fd, cL, ci, aR, x, dY, aE, dE, dv, M, dV, K, dQ, H, cN, dF, ex, eL, eN, dj, eG, dH, aW, az, fc, da, dg, aB, cJ, g, eE, eF, cm, eY, eM, dc, cj, cX, dW, m, du, eg, de, cZ, dt, dD, aw, cV, ez, fg, cQ, aj, ct, ee, eh, ai, eW, do, ec, ep, eT, cU, h, eV, dL, du, ey, dd, db, dx, cY, A, fe, aq, dw, cM, bJ, ej, W, dC, cO, dB, eD, fa, at, eD, eo, eQ, dl, B, ek, L, bL, ed, dA, eR, dk, eX, ew, aA, cR, cz, v, j, bX, dp, V, q, J, o, er, eA, eJ, di, eO, dn, aT, dz, bK, ae, af, dl, eu, eC, Y, ev, r, eS, en, t, el, fi, dS, ck, cT, ck, cl, ac, dh, cG, dT, cS, dy, bt, eP, dM, df, em, bS, dG, ah, eq, ad, dZ, dP, av, ds, dk, dm, fb, et, cH, cW, ff, eH, eZ, dN, dq, aF, Z, bz, dr, ek, dR, es, eU, bE, dJ, d, eB, dX, aS, cP, ei, y, fh); #c1(ABCA1) c2(XP_005251833) c3(ATP-binding cassette sub-family A member 1 isoform X4) c4(271201) c5( ) c6( ) c7(bM, T, ef, ea, e, eb, bB, fd, cL, ci, aR, x, dY, aE, dE, dv, M, dV, K, dQ, H, cN, dF, ex, eL, eN, dj, eG, dH, aW, az, fc, da, dg, aB, cJ, g, eE, eF, cm, eY, eM, dc, cj, cx, dW, m, du, eg, de, cZ, dt, dD, aw, cV, ez, fg, cQ, aj, ct, ee, eh, ai, eW, do, ec, ep, eT, cU, h, eV, dL, du, ey, dd, db, dx, cY, A, fe, aq, dw, cM, bJ, ej, W, dC, cO, dB, eD, fa, at, el, eo, eQ, dl, B, ek, L, bL, ed, dA, eR, dk, eX, ew, aA, CR, cz, v, j, bX, dp, V, q, J, o, er, eA, eJ, di, eO, dn, aT, dz, bK, ae, af, dl, eu, eC, Y, ev, r, eS, en, t, el, fi, dS, ck, cT, cK, cl, ac, dh, cG, dT, cS, dy, bt, eP, dM, df, em, bS, dG, ah, eq, ad, dZ, dP, av, ds, dk, dm, fb, et, cH, cW, ff, eH, eZ, dN, dq, aF, Z, bz, dr, ek, dR, es, eU, bE, dJ, do, eB, dX, aS, cP, ei, y, fh); #c1(ABCA1) c2(XP_011516642) c3(ATP-binding cassette sub-family A member 1 isoform X1) c4( ) c5(310070, 310071, 310072, 310073, 310074) c6( ) c7(bM, T, ef, ea, e, eb, bB, fd, cl, ci, aR, x, dY, aE, dE, dv, M, dV, K, dQ, H, cN, dF, ex, eL, eN, dj, eG, dH, aW, az, fc, da, dg, aB, cJ, g, eE, eF, cm, eY, eM, dc, cj, cx, dW, m, du, eg, de, cZ, dt, dD, aw, cV, ez, fg, cQ, aj, ct, ee, eh, ai, eW, do, ec, ep, eT, cU, h, eV, dL, dU, ey, dd, db, dx, cY, A, fe, aq, dw, cM, bJ, ej, W, dC, cO, dB, eD, fa, at, el, eo, eQ, dl, B, ek, L, bL, ed, dA, eR, dk, eX, ew, aA, cR, cz, v, j, bX, dp, V, q, J, o, er, eA, eJ, di, eO, dn, aT, dz, bK, ae, af, dl, eu, eC, Y, ev, r, eS, en, t, el, fi, dS, ck, cT, ck, cl, ac, dh, cG, dT, cS, dy, bt, eP, dM, df, em, bS, dG, ah, eq, ad, dZ, dP, av, ds, dk, dm, fb, et, cH, cW, ff, eH, eZ, dN, dq, af, Z, bz, dr, ek, dR, es, eU, bE, dJ, dO, eB, dX, aS, cP, ei, y, fh); #c1(ABCA1) c2(XP_011516646) c3(ATP-binding cassette sub-family A member 1 isoform X7) c4( ) c5(310075) c6( ) c7(bM, T, ef, ea, e, eb, bB, fd, cl, ci, aR, x, dY, aE, dE, dv, M, dV, K, dQ, H, cN, dF, ex, eL, eN, dj, eG, dH, aW, az, fc, da, dg, aB, cJ, g, eE, eF, cm, eY, eM, dc, cj, cX, dW, m, du, eg, de, cZ, dt, dD, aw, cV, ez, fg, cQ, aj, ct, ee, eh, ai, eW, do, ec, ep, eT, cU, h, eV, dL, du, ey, dd, db, dx, cY, A, fe, aq, dw, cM, bJ, ej, W, dC, cO, dB, eD, fa, at, el, eo, eQ, dl, B, ek, L, bL, ed, dA, eR, dk, eX, ew, aA, cR, cz, v, j, bX, dp, V, q, J, o, er, eA, eJ, di, eO, dn, aT, dz, bk, ae, af, dl, eu, eC, Y, ev, r, eS, en, t, el, fi, dS, ck, cT, cK, cl, ac, dh, cG, dT, cS, dy, bt, eP, dM, df, em, bS, dG, ah, eq, ad, dZ, dP, av, ds, dk, dm, fb, et, cH, cW, ff, eH, eZ, dN, dq, aF, Z, bz, dr, ek, dR, es, eU, bE, dJ, d, eB, dX, aS, cP, ei, y, fh); #c1(ABCA12) c2(NP_056472) c3(ATP-binding cassette sub-family A member 12 isoform b) c4(187662) c5(187663, 187664, 187665, 187666, 187667, 187668) c6( ) c7(fj, dD, fk, fm, Y, aW, ea, aT, cm, fn, fo, eq, K, fp, fl, af, dr); #c1(ABCA12) c2(NP_775099) c3(ATP-binding cassette sub-family A member 12 isoform a) c4(249508) c5(249509, 249510, 249511, 249512, 249513, 249514) c6( ) c7(fj, dD, fk, fm, Y, aW, ea, aT, cm, fn, fo, eq, K, fp, fl, af, dr); #c1(ABCA13) c2(NP_689914) c3(ATP-binding cassette sub-family A member 13) c4(244350) c5(244351, 244352, 244353, 244354, 244355, 244356) c6( ) c7(bS, fr, bK, ct, Z, fq); #c1(ABCA13) c2(XP_011513433) c3(ATP-binding cassette sub-family A member 13 isoform X2) c4( ) c5(305696) c6( ) c7(bS, fr, bK, ct, Z, fq); #c1(ABCA13) c2(XP_011513434) c3(ATP-binding cassette sub-family A member 13 isoform X3) c4(305697) c5(305698, 305699, 305700, 305701, 305702, 305703) c6( ) c7(bS, fr, bk, ct, Z, fq); #c1(ABCA2) c2(NP_001597) c3(ATP-binding cassette sub-family A member 2 isoform a) c4(119010, 119011, 119012) c5(119013, 119014, 119015, 119016, 119017, 119018, 119019, 119020, 119021, 119022, 119023, 119024) c6( ) c7(T, ft, af, dB, eZ, K, m, fu, cm, fs, B, aR); #c1(ABCA2) c2(NP_997698) c3(ATP-binding cassette sub-family A member 2 isoform b) c4(265689) c5(265690, 265691, 265692, 265693, 265694, 265695, 265696, 265697, 265698, 265699, 265700, 265701) c6( ) c7(T, ft, af, dB, eZ, K, m, fu, cm, fs, B, aR); #c1(ABCA2) c2(XP_006717059) c3(ATP-binding cassette sub-family A member 2 isoform X2) c4(290657) c5(290658, 290659, 290660, 290661, 290662, 290663) c6( ) c7(T, ft, af, dB, eZ, K, m, fu, cm, fs, B, aR); #c1(ABCA3) c2(NP_001080) c3(ATP-binding cassette sub-family A member 3) c4(38164, 38165) c5(38166, 38167, 38168, 38169, 38170, 38171, 38172, 38173, 38174, 38175, 38176, 38177) c6( ) c7(fE, fx, eM, bW, fG, bt, fz, fF, fH, fo, fw, fC, fl, fD, fA, fy, fv, fj, fB, aa); #c1(ABCA5) c2(NP_758424) c3(ATP-binding cassette sub-family A member 5) c4(249129, 249130) c5(249131, 249132, 249133, 249134, 249135, 249136, 249137, 249138, 249139, 249140, 249141, 249142) c6( ) c7(at, dL, X, an); #c1(ABCA6) c2(NP_525023) c3(ATP-binding cassette sub-family A member 6) c4(231172, 231173) c5(231174, 231175, 231/76, 231177, 231178, 231179, 231180) c6( ) c7(at, dL, fJ); #c1(ABCAG) c2(XP_006721856) c3(ATP-binding cassette sub-family A member 6 isoform X3) c4( ) c5(294322, 294323, 294324, 294325, 294326) c6( ) c7(at, dl, fJ); #c1(ABCA7) c2(NP_061985) c3(ATP-binding cassette sub-family A member 7) c4(201763) c5(201764, 201765, 201766, 201767, 201768, 201769, 201770, 201771, 201772, 201773) c6( ) c7(fM, W, af, fN, at, dL, j, fL, y, V, fK); #c1(ABCA7) c2(XP_006722679) c3(ATP-binding cassette sub-family A member 7 isoform X3) c4(294827) c5( ) c6( ) c7(fM, W, af, fN, at, dL, j, fL, y, V, fK); #c1(ABCA7) c2(XP_011525937) c3(ATP-binding cassette sub-family A member 7 isoform X9) c4( ) c5(320342, 320343, 320344, 320345, 320346, 320347) c6( ) c7(fM, W, af, fN, at, dL, j, fL, y, V, fK); #c1(ABCA9) c2(NP_525022) c3(ATP-binding cassette sub-family A member 9) c4(231166) c5(231167, 231168, 231169, 231170, 231171) c6( ) c7(t, ac, m); #c1(ABCA9) c2(XP_005256994) c3(ATP-binding cassette sub-family A member 9 isoform X5) c4(274633) c5(274634, 274635, 274636, 274637, 274638, 274639) c6( ) c7(t, ac, m); #c1(ABCA9) c2(XP_006721696) c3(ATP-binding cassette sub-family A member 9 isoform X3) c4( ) c5(294242) c6( ) c7(t, ac, m); #c1(ABCA9) c2(XP_011522489) c3(ATP-binding cassette sub-family A member 9 isoform X4) c4(317127) c5(317128, 317129, 317130, 317131, 317132, 317133) c6( ) c7(t, ac, m); #c1(ABCB1) c2(NP_000918) c3(multidrug resistance protein 1) c4(17181, 17182, 17183, 17184, 17185) c5(17186, 17187, 17188, 17189, 17190, 17191, 17192, 17193, 17194, 17195, 17196, 17197, 17198, 17199, 17200, 17201, 17202, 17203, 17204, 17205, 17206, 17207, 17208, 17209, 17210, (7211, 17212, 17213, 17214) c6( ) c7(gh, T, bM, aC, FR, hV, fV, if, e, al, bO, fd, bB, cL, gf, gA, ci, aY, gF, aR, ip, x, bv, gJ, aE, gH, il, ix, fS, M, dV, iv, ja, gs, K, hu, de, hH, eL, bc, hm, gr, eG, iE, aW, hC, az, hl, ih, hN, aB, g, ir, hJ, eF, cm, bw, iP, ix, y, fT, gt, eM, ii, iA, iT, go, cj, iS, k, m, gn, hh, eg, in, ik, gD, iu, fW, gL, gU, iB, gp, aw, ht, n, aj, fr, ct, cd, br, eW, ft, hw, ec, gd, ic, h, eV, hn, gl, hg, dL, hB, is, he, dx, fY, hi, ia, hT, gi, iw, ge, fQ, hQ, aq, fe, gG, cM, lN, cv, hq, iO, gY, dB, hk, hL, hj, gw, hs, hR, at, gv, gg, bD, jd, it, B, bL, jc, fM, dA, iH, eR, hU, hx, eX, fZ, aA, cz, hy, v, j, hz, bX, il, V, q, o, er, eA, ga, gM, hW, hb, aT, dz, gz, bk, jj, bq, hK, an, gS, ae, af, jf, gc, hM, iF, gP, s, iU, iZ, Y, ev, fO, r, iy, gm, en, hf, cl, gl, t, ho, f, ik, bN, dS, ck, hY, iM, ac, iC, hA, ig, hc, au, gX, fP, hr, hO, dy, ij, im, gT, bt, gO, ao, hS, gx, gV, hl, id, ie, aD, iW, ha, gN, bs, jh, iD, bS, ah, fu, iG, hF, gb, iJ, ad, hX, iC, fU, av, hd, fX, ib, ds, io, hD, iY, hP, ji, fb, iL, iz, hZ, gj, d, je, hG, cH, et, ff, gK, gC, eH, eZ, gl, gE, hp, gu, aF, iV, gW, Z, iR, bz jb, jg, iq, bF, es, gk, bE, gR, dJ, X, eB, gZ, gy, gB, dX, hE, gq, hv); #c1(ABCB11) c2(NP_003733) c3(bile salt export pump) c4(140518) c5(140519, 140520, 140521, 140522, 140523, 140524, 140525) c6( ) c7(T, jv, jn, jl, jp, al, jx, n, jA, j, bX, jw, l, jm, F, u, jz, ju, jk, aT, jy, dL, jo, dF, bc, aW, fc, hl, dq, hN, aq, hJ, jt, js, t, dr, jq, gk, at, m, ei, B, jr); #c1(ABCB4) c2(NP_000434) c3(multidrug resistance protein 3 isoform A) c4(8945) c5(8946, 8947, 8948, 8949, 8950, 8951, 8952, 8953, 8954, 8955, 8956) c6( ) c7(jD, T, jv, jl, al, jC, jN, fd, n, j, jE, jG, bX, jO, hF, il, jw, jF, bv, x, l, jm, F, u, jL, aT, jH, jl, bK, bq, jJ, bc, jK, d, aW, hl, jB, hQ, hN, aq, hJ, t, ho, cm, jq, ck, gk, dB, cj, m, jM, hE, B, ao); #c1(ABCB4) c2(NP_061337) c3(multidrug resistance protein 3 isoform B) c4(200145) c5(200146, 200147, 200148, 200149, 200150, 200151, 200152, 200153, 200154, 200155, 200156, 200157, 200158, 200159, 200160, 200161, 200162, 200163, 200164, 200165, 200166, 200167, 200168, 200169, 200170, 200171, 200172, 200173, 200174, 200175, 200176, 200177, 200178, 200179, 200180, 200181, 200182, 200183, 200184, 200185, 200186, 200187, 200188, 200189, 200190, 200191, 200192, 200193, 200194, 200195, 200196, 200197, 200198, 200199, 200200, 200201, 200202, 200203, 200204, 200205, 200206, 200207, 200208, 200209, 200210, 200211, 200212, 200213, 200214, 200215, 200216, 200217, 200218, 200219, 200220, 200221, 200222, 200223, 200224, 200225, 200226, 200227, 200228, 200229, 200230, 200231, 200232, 200233, 200234, 200235, 200236, 200237, 200238, 200239, 200240, 200241, 200242, 200243, 200244, 200245, 200246, 200247, 200248, 200249, 200250, 200251, 200252, 200253, 200254, 200255, 200256, 200257, 200258, 200259, 200260, 200261, 200262, 200263, 200264, 200265, 200266, 200267, 200268, 200269, 200270, 200271, 200272, 200273, 200274, 200275, 200276, 200277, 200278, 200279, 200280, 200281, 200282, 200283, 200284, 200285, 200286, 200287, 200288, 200289, 200290, 200291, 200292, 200293, 200294, 200295, 200296, 200297, 200298, 200299, 200300, 200301, 200302, 200303, 200304, 200305, 200306, 200307, 200308, 200309, 200310, 200311, 200312, 200313, 200314, 200315, 200316, 200317, 200318, 200319, 200320, 200321, 200322, 200323) c6( ) c7(jD, T, jv, jl, al, jC, jN, fd, n, j, jE, jG, bX, jO, hF, il, jw, jF, bv, x, l, jm, F, u, jL, aT, jH, jl, bK, bq, jJ, bc, jK, d, aW, hl, jB, hQ, hN, aq, hJ, t, ho, cm, jq, ck, gk, dB, cj, m, jM, hE, B, ao); #c1(ABCB4) c2(NP_061338) c3(multidrug resistance protein 3 isoform C) c4(200324, 200325) c5(200326, 200327, 200328, 200329, 200330, 200331, 200332, 200333, 200334, 200335, 200336, 200337, 200338, 200339, 200340, 200341, 200342) c6( ) c7(jD, T, jv, jl, al, jC, jN, fd, n, j, jE, jG, bX, jO, hF, il, jw, jF, bv, x, l, jm, F, u, jL, aT, jH, jl, bK, bq, jJ, bc, jK, d, aW, hl, jB, hQ, hN, aq, hJ, t, ho, cm, jq, ck, gk, dB, cj, m, jM, hE, B, ao); #c1(ABCB4) c2(XP_011514616) c3 (multidrug resistance protein 3 isoform X7) c4( ) c5(307306, 307307) c6( ) c7(jD, T, jv, jl, al, jC, jN, fd, n, j, jE, jG, bX, jO, hF, il, jw, jF, bv, x, l, jm, F, u, jL, aT, jH, jl, bK, bq, jJ, bc, jK, d, aW, hl, jB, hQ, hN, aq, hJ, t, ho, cm, jq, ck, gk, dB, cj, m, jM, hE, B, ao); #c1(ABCB5) c2(NP_001157413) c3(ATP-binding cassette sub-family B member 5 isoform 1) c4(62171) c5(62172, 62173, 62174, 62175, 62176, 62177, 62178, 62179, 62180, 62181, 62182, 62183) c6( ) c7(T, ha, jh, fd, n, v, j, ee, hF, x, aT, hH, iU, aW, Y, aq, cJ, iR, ck, cj, k, hE, bt, B); #c1(ABCB5) c2(NP_001157414) c3(ATP-binding cassette sub-family B member 5 isoform 3) c4( ) c5(62184, 62185, 62186, 62187, 62188) c6( ) c7(T, ha, jh, fd, n, v, j, ee, hF, x, aT, hH, iU, aW, Y, aq, cJ, iR, ck, cj, k, hE, bt, B); #c1(ABCB5) c2(NP_001157465) c3(ATP-binding cassette sub-family B member 5 isoform 4) c4(62224) c5(62225, 62226, 62227, 62228, 62229) c6( ) c7(T, ha, jh, fd, n, v, j, ee, hF, x, aT, hH, iU, aW, Y, aq, cJ, iR, ck, cj, k, hE, bt, B); #c1(ABCB5) c2(NP_848654) c3(ATP-binding cassette sub-family B member 5 isoform 2) c4(254088) c5( ) c6( ) c7(T, ha, jh, fd, n, v, j, ee, hF, x, aT, hH, iU, aW, Y, aq, cJ, iR, ck, cj, k, hE, bt, B); #c1(ABCB5) c2(XP_006715785) c3(ATP-binding cassette sub-family B member 5 isoform X2) c4(289789) c5(289790, 289791) c6( ) c7(T, ha, jh, fd, n, v, j, ee, hF, x, aT, hH, iU, aW, Y, aq, cJ, iR, ck, cj, k, hE, bt, B); #c1(ABCB6) c2(NP_005680) c3(ATP-binding cassette sub-family B member 6, mitochondrial) c4(158477, 158478, 158479) c5(158480, 158481, 158482, 158483, 158484, 158485, 158486, 158487, 158488, 158489, 158490, 158491, 158492, 158493, 158494, 158495, 158496, 158497) c6( ) c7(T, jW, ea, al, bB, fo, kb, ci, jY, ku, dV, aU, K, dF, eL, aW, hl, kt, cJ, eE, cm, jV, ki, m, cZ, dt, dD, gp, n, ko, kv, jX, ke, cc, h, dl, dd, kj, kw, jP, ks, aq, kp, kk, jT, kn, dB, B, fl, bL, ka, jx, aA, eA, eJ, hb, jZ, aT, kf, kr, af, dl, kl, iU, Y, en, t, jU, dS, ck, kc, km, jQ, ao, jR, kh, ha, eq, hX, dP, ds, ff, aF, hp, iV, iR, bz, dr, jS, gk, kg, bE, kq, ei, hE, kd); #c1(ABCB7) c2(NP_001258625) c3(ATP-binding cassette sub-family B member 7, mitochondrial isoform 2) c4(96199, 96200) c5(96201, 96202, 96203, 96204, 96205, 96206) c6( ) c7(kx, ky, kB, kA, kz, kD, kC); #c1(ABCB7) c2(NP_001258626) c3(ATP-binding cassette sub-family B member 7, mitochondrial isoform 3) c4(96207) c5(96208, 96209, 96210, 96211, 96212, 96213) c6( ) c7(kx, ky, kB, kA, kz, kD, kC); #c1(ABCB7) c2(NP_001258627) c3(ATP-binding cassette sub-family B member 7, mitochondrial isoform 4) c4(96214, 96215) c5(96216, 96217, 96218, 96219, 96220, 96221, 96222, 96223, 96224, 96225, 96226, 96227) c6( ) c7(kx, ky, kB, KA, kz, kD, KC); #c1(ABCB7) c2(NP_004290) c3(ATP-binding cassette sub-family B member 7, mitochondrial isoform 1) c4(144887) c5(144888, 144889, 144890, 144891, 144892, 144893) c6( ) c7(kx, ky, kB, kA, kz, kD, kC); #c1(ABCB8) c2(NP_001269220) c3(ATP-binding cassette sub-family B member 8, mitochondrial isoform a) c4(100801, 100802) c5(100803, 100804, 100805, 100806, 100807, 100808, 100809, 100810, 100811, 100812, 100813, 100814) c6( ) c7(T, Y); #c1(ABCB8) c2(NP_001269221) c3(ATP-binding cassette sub-family B member 8, mitochondrial isoform c) c4(100815, 100816) c5(100817, 100818, 100819, 100820, 100821, 100822, 100823, 100824, 100825, 100826, 100827, 100828) c6( ) c7(T, Y); #c1(ABCB8) c2(NP_001269222) c3(ATP-binding cassette sub-family B member 8, mitochondrial isoform d) c4(100829) c5(100830, 100831, 100832, 100833, 100834, 100835) c6( ) c7(T, Y); #c1(ABCB8) c2(NP_009119) c3(ATP-binding cassette sub-family B member 8, mitochondrial isoform b) c4(170823, 170824) c5(170825, 170826, 170827, 170828, 170829, 170830, 170831, 170832, 170833, 170834, 170835, 170836) c6( ) c7(T, Y); #c1(ABCB9) c2(NP_001229942) c3(ATP-binding cassette sub-family B member 9 isoform 6) c4(85520) c5(85521, 85522, 85523, 85524, 85525, 85526) c6( ) c7(eM); #c1(ABCB9) c2(NP_001229943) c3(ATP-binding cassette sub-family B member 9 isoform 5) c4( ) c5(85527, 85528, 85529, 85530, 85531, 85532) c6( ) c7(eM); #c1(ABCB9) c2(NP_062570) c3(ATP-binding cassette sub-family B member 9 isoform 2) c4(202005) c5(202006, 202007, 202008, 202009, 202010, 202011) c6( ) c7(eM); #c1(ABCB9) c2(NP_062571) c3(ATP-binding cassette sub-family B member 9 isoform 1) c4(202012, 202013, 202014) c5(202015, 202016, 202017, 202018, 202019, 202020, 202021, 202022, 202023, 202024, 202025, 202026, 202027, 202028, 202029, 202030, 202031, 202032, 202033, 202034) c6( ) c7(BM); #c1(ABCB9) c2(NP_982269) c3(ATP-binding cassette sub-family B member 9 isoform 4) c4(263241) c5(263242, 263243, 263244, 263245, 263246, 263247) c6( ) c7(BM); #c1(ABCC1) c2(NP_004987) c3(multidrug resistance-associated protein 1) c4(151717, 151718, 15/719, 15/720, 15/721) c5(151722, 151723, 151724, 151725, 15/726, 15/727, 151728, 151729, 151730, 151731, 151732, 151733, 151734, 151735, 151736, 151737, 151738, 151739, 151740, 151741, 151742, 151743, 151744, 151745, 151746, 151747, 151748, 151749, 151750, 151751, 151752, 151753, 151754, 151755, 151756, 151757, 151758, 151759, 151760, 15/761, 15/762, 15/763, 151764) c6( ) c7(bM, T, aC, bO, fd, bB, fo, ci, x, bv, aE, dV, K, hu, eL, bc, kF, aW, as, g, eF, kJ, ir, cm, eM, cj, iS, m, k, gn, ik, gD, iu, dD, iB, ht, n, kM, fr, kO, ft, kl, kH, h, dL, ia, kE, kK, hQ, aq, cM, dB, hL, at, B, fl, aA, cz, hy, bX, q, er, eA, aT, ae, af, jf, gc, iU, iZ, Y, ev, r, en, hf, cl, t, kC, ho, hY, ck, iM, ac, gX, bt, ao, kG, hS, gV, hl, ha, jh, eq, hF, iJ, ad, kL, av, ib, fb, d, je, hG, ff, eH, cr, eZ, kD, aF, iR, bz, kN, dr, bE, gZ, y, X); #c1(ABCC1) c2(XP_011520799) c3(multidrug resistance-associated protein 1 isoform X1) c4(315808, 315809) c5( ) c6( ) c7(bM, T, aC, bO, fd, bB, fo, ci, x, bv, aE, dV, K, hu, eL, bc, kF, aW, as, g, eF, kJ, ir, cm, eM, cj, iS, m, k, gn, ik, gD, iu, dD, iB, ht, n, kM, fr, KO, ft, kl, kH, h, dL, ia, kE, kK, hQ, aq, cM, dB, hL, at, B, fl, aA, cz, hy, bX, q, er, eA, aT, ae, af, jf, gc, iU, iZ, Y, ev, r, en, hf, cl, t, kC, ho, hY, ck, iM, ac, gX, bt, ao, kG, hS, gV, hl, ha, jh, eq, hF, iJ, ad, kL, av, ib, fb, d, je, hG, ff, eH, cr, eZ, kD, aF, iR, bz, kN, dr, bE, gZ, y, X); #c1(ABCC10) c2(NP_001185863) c3(multidrug resistance-associated protein 7 isoform MRP7) c4(76594) c5(76595, 76596, 76597, 76598, 76599, 76600) c6( ) c7(av, kP, eM, aW, kQ, aT, h, gA, hy, K, g); #c1(ABCC10) c2(NP_258261) c3(multidrug resistance-associated protein 7 isoform MRP7A) c4(228690) c5(228691, 228692, 228693, 228694, 228695, 228696) c6( ) c7(av, kP, eM, aW, kQ, aT, h, gA, hy, K, g); #c1(ABCC10) c2(XP_011513282) c3(multidrug resistance-associated protein 7 isoform X6) c4( ) c5(305551) c6( ) c7(av, kP, eM, aW, kQ, aT, h, gA, hy, K, g); #c1(ABCC11) c2(NP_115972) c3(ATP-binding cassette sub-family C member 11 isoform a) c4(225013) c5(225014, 225015, 225016, 225017, 225018, 225019, 225020, 225021, 225022, 225023, 225024, 225025, 225026, 225027) c6( ) c7(kV, bL, aj, v, C, KW, KY, J, ft, lg, ld, aT, K, jj, kU, H, ao, li, le, A, hG, aW, kX, lb, lh, la, eS, lc, bz, kR, ck, kT, y, iT, dh, cj, cx, dy, kZ, lf, bt, kS, hv, L); #c1(ABCC11) c2(NP_660187) c3(ATP-binding cassette sub-family C member II isoform b) c4(240489, 240490) c5(240491, 240492, 240493, 240494, 240495, 240496, 240497, 240498, 240499, 240500, 240501, 240502) c6( ) c7(kV, bL, aj, v, C, kW, KY, J, ft, lg, ld, aT, K, jj, kU, H, ao, li, le, A, hG, aW, kX, lb, lh, la, eS, lc, bz, kR, ck, KT, y, iT, dh, cj, cX, dy, kZ, lf, bt, kS, hv, L); #c1(ABCC12) c2(NP_150229) c3(multidrug resistance-associated protein 9) c4(227795, 227796, 227797, 227798, 227799, 227800) c5(227801, 227802, 227803, 227804, 227805, 227806, 227807, 227808, 227809, 227810, 227811, 227812, 227813, 227814, 227815, 227816, 227817, 227818, 227819, 227820, 227821, 227822, 227823, 227824, 227825, 227826, 227827, 227828, 227829, 227830, 227831, 227832, 227833, 227834, 227835, 227836) c6( ) c7(aW, aT); #c1(ABCC2) c2(NP_000383) c3(canalicular multispecific organic anion transporter 1) c4(8350) c5(8351, 8352, 8353, 8354, 8355, 8356) c6( ) c7(bL, gV, T, jv, fM, lk, aC, eR, fV, ha, jx, lr, it, ll, n, lj, jh, fr, ci, bX, Iu, aY, jw, q, aR, av, gJ, F, ft, ib, aE, lt, jz, fS, jL, aT, h, dL, hg, jo, bq, bc, ln, ls, eG, hG, aW, Y, KK, hC, hl, eZ, r, fc, ff, lo, af, lq, en, hf, lm, t, hJ, bz, g, iK, jq, es, iO, eM, iA, lp, go, kQ, X, gZ, at, dy, m, hE, bt, cB, B, y, hS); #c1(ABCC2) c2(XP_006717694) c3(canalicular multispecific organic anion transporter 1 isoform X3) c4( ) c5(291299, 291300, 291301, 291302, 291303, 291304) c6( ) c7(bL, gV, T, jv, fM, lk, aC, eR, fV, ha, jx, lr, it, ll, n, lj, jh, fr, ci, bX, lu, aY, jw, q, aR, av, gJ, F, ft, ib, aE, lt, jz, fS, jL, aT, h, dl, hg, jo, bq, bc, ln, ls, eG, hG, aW, Y, kK, hC, hl, eZ, r, fc, ff, lo, aF, lq, en, hf, lm, t, hJ, bz, g, ik, jq, es, iO, eM, iA, lp, go, kQ, X, gZ, at, dy, m, hE, bt, cB, B, y, hS); #c1(ABCC2) c2(XP_011537593) c3(canalicular multispecific organic anion transporter 1 isoform X2) c4(335186) c5( ) c6( ) c7(bL, gV, T, jv, fM, lk, aC, eR, fV, ha, jx, lr, it, Il, n, lj, jh, fr, ci, bX, lu, aY, jw, q, aR, av, gJ, F, ft, ib, aE, lt, jz, fS, jL, aT, h, dl, hg, jo, bq, bc, ln, ls, eG, hG, aW, Y, kK, hC, hl, eZ, r, fc, ff, lo, aF, Iq, en, hf, lm, t, hJ, bz, g, iK, jq, es, iO, eM, iA, lp, go, kQ, X, gZ, at, dy, m, hE, bt, cB, B, y, hS); #c1(ABCC3) c2(NP_001137542) c3(canalicular multispecific organic anion transporter 2 isoform 2) c4(56007) c5(56008, 56009, 56010, 56011, 56012, 56013) c6( ) c7(T, aC, eR, al, jh, aA, bB, n, cL, ci, bX, hF, q, o, eA, x, av, l, ft, ib, u, aE, lv, jL, aT, dL, hP, K, eL, bc, ls, d, je, s, hG, iE, aW, Y, kK, hC, hl, eH, r, eZ, aq, fc, aF, g, as, hJ, t, eF, bz, cm, ik, jq, ck, gk, iM, eM, dB, bE, cj, dy, k, lw, hE, bt, B, y); #c1(ABCC3) c2(NP_003777) c3(canalicular multispecific organic anion transporter 2 isoform 1) c4(140748, 140749) c5(140750, 140751, 140752, 140753, 140754, 140755, 140756, 140757, 140758, 140759, 140760, 140761) c6( ) c7(T, aC, eR, al, jh, aA, bB, n, cL, ci, bX, hF, q, o, eA, x, av, l, ft, ib, u, aE, lv, jL, aT, dl, hP, K, eL, bc, ls, d, je, s, hG, iE, aW, Y, kK, hC, hl, eH, r, eZ, aq, fc, aF, g, as, hJ, t, eF, bz, cm, ik, jq, ck, gk, iM, eM, dB, bE, cj, dy, k, lw, hE, bt, B, y); #c1(ABCC3) c2(XP_011523725) c3(canalicular multispecific organic anion transporter 2 isoform X3) c4( ) c5(318184) c6( ) c7(T, aC, eR, al, jh, aA, bB, n, cl, ci, bX, hF, q, o, eA, x, av, l, ft, ib, u, aE, lv, jL, aT, dL, hP, K, eL, bc, ls, d, je, s, hG, iE, aW, Y, kK, hC, hl, eH, r, eZ, aq, fc, aF, g, as, hJ, t, eF, bz, cm, ik, jq, ck, gk, iM, eM, dB, bE, cj, dy, k, lw, hE, bt, B, y); #c1(ABCC4) c2(NP_001098985) c3(multidrug resistance-associated protein 4 isoform 2) c4(41569) c5(41570, 41571, 41572, 41573, 41574, 41575) c6( ) c7(ID, aC, aA, n, gA, bX, aY, q, ad, dP, av, x, ft, ib, lz, aT, h, dL, H, ae, bc, ly, d, B, lE, hG, ev, ge, r, aF, g, iV, hJ, lA, cm, bz, lx, ck, kQ, bt, lF, at, aS, k, dy, lC, lB, it, X, L); #c1(ABCC4) c2(NP_001288758) c3(multidrug resistance-associated protein 4 isoform 3) c4(113979) c5(113980, 113981, 113982, 113983, 113984, 113985) c6( ) c7(ID, aC, aA, n, gA, bX, aY, q, ad, dP, av, x, ft, ib, lz, aT, h, dL, H, ae, bc, ly, d, B, lE, hG, ev, ge, r, aF, g, iV, hJ, lA, cm, bz, lx, ck, kQ, bt, lF, at, aS, k, dy, IC, lB, it, X, L); #c1(ABCC4) c2(NP_001288759) c3(multidrug resistance-associated protein 4 isoform 4) c4(113986, 113987) c5(113988, (13989, 113990, 113991, 113992, 113993) c6( ) c7(ID, aC, aA, n, gA, bX, aY, q, ad, dP, av, x, ft, ib, lz, aT, h, dL, H, ae, bc, ly, d, B, lE, hG, ev, ge, r, aF, g, iV, hJ, lA, cm, bz, lx, ck, kQ, bt, lF, at, aS, k, dy, lC, lB, it, X, L); #c1(ABCC4) c2(NP_005836) c3(multidrug resistance-associated protein 4 isoform 1) c4(159778) c5(159779, 159780, 159781, 159782, 159783, 159784, 159785, 159786, 159787, 159788, 159789, 159790) c6( ) c7(ID, aC, aA, n, gA, bX, aY, q, ad, dP, av, x, ft, ib, lz, aT, h, dL, H, ae, bc, ly, d, B, lE, hG, ev, ge, r, aF, g, iV, hJ, lA, cm, bz, lx, ck, kQ, bt, IF, at, aS, k, dy, lC, lB, it, X, L); #c1(ABCC5) c2(NP_001018881) c3(multidrug resistance-associated protein 5 isoform 2) c4(27570) c5(27571, 27572, 27573, 27574, 27575, 27576, 27577, 27578, 27579, 27580, 27581, 27582, 27583, 27584, 27585, 27586, 27587, 27588, 27589, 27590, 27591, 27592, 27593, 27594, 27595, 27596, 27597, 27598, 27599, 27600) c6( ) c7(aC, lH, ha, ci, bX, eA, eJ, ip, x, ft, cc, aE, gd, aT, lG, K, bc, d, aW, g, t, bz, eM, ll, bt, m, k, lJ, X); #c1(ABCC5) c2(NP_005679) c3(multidrug resistance-associated protein 5 isoform 1) c4(158453, 158454, 158455, 158456, 158457, 158458) c5(158459, 158460, 158461, 158462, 158463, 158464, 158465, 158466, 158467, 158468, 158469, 158470, 158471, 158472, 158473, 158474, 158475, 158476) c6( ) c7(aC, lH, ha, ci, bX, eA, eJ, ip, x, ft, cc, aE, gd, aT, IG, K, bc, d, aW, g, t, bz, eM, ll, bt, m, k, lJ, X); #c1(ABCC5) c2(XP_011510617) c3(multidrug resistance-associated protein 5 isoform X2) c4(301075) c5( ) c6( ) c7(aC, lH, ha, ci, bX, eA, eJ, ip, x, ft, cc, aE, gd, aT, lG, K, bc, d, aW, g, t, bz, eM, ll, bt, m, k, lJ, X); #c1(ABCC6) c2(NP_001072996) c3(URG7 protein isoform 2) c4(35547) c5(35548, 35549, 35550, 35551, 35552, 35553) c6( ) c7(bL, e, cz, aj, fo, j, jE, bX, eA, J, lK, lT, M, K, lQ, bc, hm, gc, lR, lL, ih, lP, eZ, jP, ax, dq, g, dr, lU, ck, lN, lO, dx, aS, lS, ei, lM, y, bt, fl); #c1(ABCC6) c2(XP_011520781) c3(multidrug resistance-associated protein 6 isoform X2) c4(315792) c5( ) c6( ) c7(bL, e, cz, aj, fo, j, jE, bX, eA, J, lK, IT, M, K, lQ, bc, hm, gc, lR, lL, ih, lP, eZ, jP, ax, dq, g, dr, lU, ck, lN, lO, dx, aS, lS, ei, lM, y, bt, fl); #c1(ABCC6) c2(XP_011520783) c3(multidrug resistance-associated protein 6 isoform X1) c4(315793) c5( ) c6( ) c7(bL, e, cz, aj, fo, j, jE, bX, eA, J, lK, lT, M, K, lQ, bc, hm, gc, lR, lL, ih, lP, eZ, jP, ax, dq, g, dr, lU, ck, lN, lO, dX, aS, lS, ei, lM, y, bt, fl); #c1(ABCC6) c2(XP_011520784) c3(multidrug resistance-associated protein 6 isoform X3) c4(315794) c5(315795, 315796, 315797, 315798, 315799, 315800) c6( ) c7(bL, e, cz, aj, fo, j, jE, bX, eA, J, lK, lT, M, K, lQ, bc, hm, gc, lR, lL, ih, lP, eZ, jP, ax, dq, g, dr, lU, ck, lN, lO, dx, aS, lS, ei, lM, y, bt, fl); #c1(ABCC8) c2(NP_000343) c3(ATP-binding cassette sub-family C member 8 isoform 2) c4(7431, 7432, 7433) c5(7434, 7435, 7436, 7437, 7438, 7439, 7440, 7441, 7442, 7443, 7444, 7445, 7446, 7447, 7448, 7449, 7450, 7451) c6( ) c7(kV, bL, eR, lV, e, ha, ml, aj, dG, v, mc, C, kY, cd, J, me, md, mj, mh, lg, ld, aT, jy, K, jo, jj, kU, H, af, ao, li, le, A, s, hG, aW, lW, kX, lb, lh, la, lY, aq, kp, mb, bz, dr, ek, ck, kT, y, ac, mi, ma, iT, bo, dh, mg, lZ, cj, lX, mf, dy, mk, ei, kZ, lf, KS, B, hv, L); #c1(ABCC8) c2(NP_001274103) c3(ATP-binding cassette sub-family C member 8 isoform 1) c4(106022) c5(106023, 106024, 106025, 106026, 106027, 106028) c6( ) c7(kV, bL, eR, lV, e, ha, ml, aj, dG, v, mc, C, kY, cd, J, me, md, mj, mh, lg, ld, aT, jy, K, jo, jj, kU, H, af, ao, li, le, A, s, hG, aW, lW, kX, lb, lh, la, lY, aq, kp, mb, bz, dr, ek, ck, kT, y, ac, mi, ma, iT, bo, dh, mg, lZ, cj, lX, mf, dy, mk, ei, kZ, lf, KS, B, hv, L); #c1(ABCC9) c2(NP_005682) c3(ATP-binding cassette sub-family C member 9 isoform SURZA) c4(158512, 158513) c5(158514, 158515, 158516, 158517, 158518, 158519, 158520, 158521, 158522, 158523, 158524, 158525) c6( ) c7(mr, ms, e, aw, cV, cz, mw, bS, gf, ct, mp, cd, dP, cc, mm, af, hm, mu, mq, mo, Z, mv, mx, hL, gZ, mt, mn); #c1(ABCC9) c2(NP_064693) c3(ATP-binding cassette sub-family C member 9 isoform SUR2B) c4(203465, 203466) c5(203467, 203468, 203469, 203470, 203471, 203472) c6( ) c7(mr, ms, e, aw, cV, cz, mw, bS, gf, ct, mp, cd, dP, cc, mm, af, hm, mu, mq, mo, Z, mv, mx, hL, gZ, mt, mn); #c1(ABCC9) c2(XP_005253347) c3(ATP-binding cassette sub-family C member 9 isoform X5) c4( ) c5(272430, 272431, 272432, 272433, 272434, 272435) c6( ) c7(mr, ms, e, aw, cV, cz, mw, bS, gf, ct, mp, cd, dP, cc, mm, af, hm, mu, mq, mo, Z, mv, mx, hL, gZ, mt, mn); #c1(ABCD1) c2(NP_000024) c3(ATP-binding cassette sub-family D member 1) c4(225, 226, 227) c5(228, 229, 230, 231, 232, 233, 234, 235, 236, 237, 238, 239, 240, 241, 242, 243, 244, 245) c6( ) c7(cZ, T, dD, jx, cz, eq, ml, bX, mF, ee, mC, aR, x, aX, h, mL, K, dd, he, dF, af, mJ, mH, ao, iU, mD, mA, eZ, dq, mB, dr, mK, dR, ck, mG, U, mE, mz, my, fl, hh, jr); #c1(ABCD2) c2(XP_011536329) c3(ATP-binding cassette sub-family D member 2 isoform X1) c4(333673) c5(333674, 333675, 333676, 333677, 333678, 333679) c6( ) c7(mD, aW, U, aT, mL, dF, B); #c1(ABCD3) c2(XP_011540179) c3(ATP-binding cassette sub-family D member 3 isoform X3) c4( ) c5(338557, 338558, 338559, 338560, 338561, 338562) c6( ) c7(mD, dF, mL, ci); #c1(ABCD4) c2(NP_005041) c3(ATP-binding cassette sub-family D member 4) c4(152388) c5(152389, 152390, 152391, 152392, 152393, 152394, 152395, 152396, 152397, 152398, 152399, 152400) c6( ) c7(mD, mN, dF, mO, mM); #c1(ABCD4) c2(XP_005268010) c3(ATP-binding cassette sub-family D member 4 isoform X6) c4(281413) c5(281414, 281415, 281416, 281417, 281418, 281419) c6( ) c7(mD, mN, dF, mO, mM); #c1(ABCD4) c2(XP_006720286) c3(ATP-binding cassette sub-family D member 4 isoform X1) c4(293343) c5( ) c6( ) c7(mD, mN, dF, mO, mM); #c1(ABCE1) c2(NP_002931) c3(ATP-binding cassette sub-family E member 1) c4(133693, 133694) c5(133695, 133696, 133697, 133698, 133699, 133700, 133701, 133702, 133703, 133704, 133705, 133706) c6( ) c7(T, F, dA, hl, jh, h, ey, X, ad); #c1(ABCF1) c2(NP_001020262) c3(ATP-binding cassette sub-family F member 1 isoform a) c4(28399, 28400, 28401, 28402, 28403) c5(28404, 28405, 28406, 28407, 28408, 28409, 28410, 28411, 28412, 28413, 28414, 28415, 28416, 28417, 28418, 28419, 28420, 28421, 28422, 28423, 28424, 28425, 28426, 28427, 28428, 28429, 28430, 28431, 28432, 28433, 28434, 28435, 28436, 28437, 28438, 28439, 28440, 28441, 28442, 28443, 28444, 28445, 28446, 28447, 28448, 28449, 28450, 28451, 28452, 28453, 28454, 28455, 28456, 28457, 28458, 28459, 28460, 28461, 28462, 28463, 28464, 28465, 28466, 28467, 28468, 28469, 28470, 28471, 28472, 28473, 28474, 28475, 28476, 28477, 28478, 28479, 28480) c6( ) c7(ar, mR, mP, mQ, eZ, HD, fD); #c1(ABCF1) c2(NP_001081) c3(ATP-binding cassette sub-family F member 1 isoform b) c4(38178) c5(38179, 38180, 38181, 38182, 38183, 38184, 38185, 38186, 38187, 38188, 38189, 38190, 38191, 38192, 38193, 38194, 38195, 38196, 38197, 38198, 38199, 38200, 38201, 38202, 38203, 38204, 38205, 38206, 38207, 38208, 38209, 38210, 38211, 38212, 38213, 38214, 38215, 38216, 38217, 38218, 38219, 38220, 38221, 38222, 38223, 38224, 38225, 38226) c6( ) c7(ar, mR, mP, mQ, eZ, hD, fD); #c1(ABCF2) c2(NP_005683) c3(ATP-binding cassette sub-family F member 2 isoform b) c4(158526) c5(158527, 158528, 158529, 158530, 158531, 158532, 158533) c6( ) c7(T, hw, hl, g, ir, lu, c1); #c1(ABCF2) c2(NP_009120) c3(ATP-binding cassette sub-family F member 2 isoform a) c4(170837, 170838, 170839) c5(170840, 170841, 170842, 170843, 170844, 170845, 170846, 170847, 170848, 170849, 170850, 170851, 170852, 170853, 170854, 170855, 170856) c6( ) c7(T, hw, hl, g, ir, lu, c1); #c1(ABCG1) c2(NP_004906) c3(ATP-binding cassette sub-family G member 1 isoform 4) c4(150684, 150685) c5(150686, 150687, 150688, 150689, 150690, 150691, 150692, 150693, 150694, 150695, 150696, 150697, 150698, 150699, 150700, 150701, 150702, 150703) c6( ) c7(bL, dD, eR, ea, e, aw, cR, mS, j, eq, bX, ad, cF, ib, mW, dn, aT, dz, bK, da, mY, mV, ae, af, na, aa, mU, et, cH, aW, kp, f, mZ, dr, mX, W, nb, dT, aS, mT); #c1(ABCG1) c2(NP_058198) c3(ATP-binding cassette sub-family G member 1 isoform 2) c4(192667) c5(192668, 192669, 192670, 192671, 192672, 192673) c6( ) c7(bL, dD, eR, ea, e, aw, cR, mS, j, eq, bX, ad, cF, ib, mW, dn, aT, dz, bK, dQ, mY, mV, ae, af, na, aa, ml, et, cH, aW, kp, f, mZ, dr, mX, W, nb, dT, aS, mT); #c1(ABCG1) c2(NP_997057) c3(ATP-binding cassette sub-family G member 1 isoform 3) c4(265005) c5(265006, 265007, 265008, 265009, 265010, 265011) c6( ) c7(bL, dD, eR, ea, e, aw, cR, mS, j, eq, bX, ad, cF, ib, mW, dn, aT, dz, bk, dl, mY, mV, ae, af, na, aa, mU, et, cH, aW, kp, f, mZ, dr, mx, W, nb, dT, aS, mT); #c1(ABCG1) c2(NP_997510) c3(ATP-binding cassette sub-family G member 1 isoform 5) c4(265572) c5(265573, 265574, 265575, 265576, 265577, 265578) c6( ) c7(bL, dD, eR, ea, e, aw, cR, mS, j, eq, bX, ad, cF, ib, mW, dn, aT, dz, bK, dl, mY, mV, ae, af, na, aa, mU, et, cH, aW, kp, f, mZ, dr, mX, W, nb, dT, aS, mT); #c1(ABCG1) c2(NP_997512) c3(ATP-binding cassette sub-family G member 1 isoform 7) c4(265579) c5(265580, 265581, 265582, 265583, 265584, 265585) c6( ) c7(bL, dD, eR, ea, e, aw, cR, mS, j, eq, bX, ad, cF, ib, mW, dn, aT, dz, bK, dQ, mY, mV, ae, af, na, aa, mU, et, cH, aW, kp, f, mZ, dr, mx, W, nb, dT, aS, mT); #c1(ABCG1) c2(XP_011528108) c3(ATP-binding cassette sub-family G member 1 isoform X1) c4(323199) c5( ) c6( ) c7(bL, dD, eR, ea, e, aw, cR, mS, j, eq, bX, ad, cF, ib, mW, dn, aT, dz, bK, de, mY, mV, ae, af, na, aa, mU, et, cH, aW, kp, f, mZ, dr, mX, W, nb, dT, aS, mT); #c1(ABCG2) c2(NP_001244315) c3(ATP-binding cassette sub-family G member 2 isoform 2) c4(92132) c5(92133, 92134, 92135, 92136, 92137, 92138) c6( ) c7(ng, gh, T, aC, fV, ea, e, al, fd, nq, bB, gA, fo, ci, mF, aR, x, gJ, aE, M, K, H, dF, bc, eG, aW, g, lm, nr, hJ, cm, eM, nB, iT, nF, cj, nw, k, m, gD, iu, nD, dD, aw, n, fr, nA, nE, ee, cd, ft, no, cc, eT, gd, h, lG, nm, ni, cY, nP, ny, kK, fe, aq, kp, nN, jq, nu, W, bP, dB, nj, gw, mE, nL, nz, cB, B, fl, it, L, bL, fM, nx, aA, hy, v, j, V, nt, q, eA, kx, nc, nv, hb, ns, aT, cy, bq, ae, ne, af, eu, nn, s, nO, iU, Y, fO, r, lo, gm, en, hf, t, nC, f, ik, ck, nM, ac, nG, np, im, bt, ao, gV, nl, ha, jh, iD, eq, hF, ad, KL, iC, av, nH, l, ib, nd, nl, iY, lQ, fb, nJ, iL, iz, d, lR, hG, ff, nh, nK, nf, cr, aF, nk, iV, bz, dr, iq, es, gk, nb, bE, aS, i, HE, X, nQ); #c1(ABCG2) c2(NP_004818) c3(ATP-binding cassette sub-family G member 2 isoform 1) c4(150033) c5(150034, 150035, 150036, 150037, 150038, 150039) c6( ) c7(ng, gh, T, aC, fV, ea, e, al, fd, nq, bB, gA, fo, ci, mF, aR, x, gJ, aE, M, K, H, dF, bc, eG, aW, g, lm, nr, hJ, cm, eM, nB, iT, nF, cj, nw, k, m, gD, iu, nD, dD, aw, n, fr, nA, nE, ee, cd, ft, no, cc, eT, gd, h, lG, nm, ni, cY, nP, ny, kK, fe, aq, kp, nN, jq, nu, W, bP, dB, nj, gw, mE, nL, nz, cB, B, fl, it, L, bL, fM, nx, aA, hy, v, j, V, nt, q, eA, kx, nc, nv, hb, ns, aT, cy, bq, ae, ne, af, eu, nn, s, no, iU, Y, fO, r, lo, gm, en, hf, t, nC, f, iK, ck, nM, ac, nG, np, im, bt, ao, gV, nl, ha, jh, iD, eq, hF, ad, kL, iC, av, nH, l, ib, nd, nl, iY, lQ, fb, nJ, iL, iz, d, lR, hG, ff, nh, nk, nf, cr, aF, nk, iV, bz, dr, iq, es, gk, nb, bE, aS, i, hE, X, nQ); #c1(ABCG4) c2(NP_001135977) c3(ATP-binding cassette sub-family G member 4) c4(53916) c5(53917, 53918, 53919, 53920, 53921, 53922, 53923, 53924, 53925, 53926, 53927, 53928, 53929, 53930) c6( ) c7(cH, eq, dD, af); #c1(ABCG5) c2(NP_071881) c3(ATP-binding cassette sub-family G member 5) c4(212049) c5(212050, 212051, 212052, 212053, 212054, 212055) c6( ) c7(dD, ea, e, n, j, eq, nS, ad, jF, l, nV, u, ec, bq, dQ, af, nU, cH, Y, da, hJ, dC, nR, nT, ei); #c1(ABCG5) c2(XP_011531328) c3(ATP-binding cassette sub-family G member 5 isoform X4) c4(327327, 327328) c5(327329, 327330, 327331, 327332, 327333, 327334) c6( ) c7(dD, ea, e, n, j, eq, nS, ad, jF, l, nV, u, ec, bq, dQ, af, nU, cH, Y, da, hJ, dC, nR, nT, ei); #c1(ABCG5) c2(XP_011531330) c3(ATP-binding cassette sub-family G member 5 isoform X7) [4( ) c5(327335, 327336, 327337, 327338, 327339, 327340) c6( ) c7(dD, ea, e, n, j, eq, nS, ad, jF, l, nV, u, ec, bq, de, af, nU, cH, Y, da, hJ, dC, nR, nT, ei); #c1(ABCG8) c2(NP_071882) c3(ATP-binding cassette sub-family G member 8) c4(212056) c5(212057, 212058, 212059, 212060, 212061, 212062) c6( ) c7(bL, nZ, dD, ea, e, n, dG, j, eq, nS, jF, l, u, ec, lt, nX, io, dl, bq, dQ, hB, eu, cH, nY, da, S, hJ, W, dC, nR, nW, nT, ei); #c1(ABHD(2) c2(NP_001035937) c3(monoacylglycerol lipase ABHD(2 isoform a) c4(33485, 33486) c5(33487, 33488, 33489, 33490, 33491, 33492, 33493, 33494, 33495, 33496, 33497, 33498) c6( ) c7(nk, ob, oa, U, nw, oc); #c1(ABHD(2) c2(NP_056415) c3(monoacylglycerol lipase ABHD12 isoform b) c4(187504) c5(187505, 187506, 187507, 187508, 187509, 187510) c6( ) c7(nk, ob, oa, U, nw, oc); #c1(ABHD12) c2(XP_005260756) c3(monoacylglycerol lipase ABHD12 isoform X3) c4( ) c5(276862, 276863, 276864, 276865, 276866, 276867) c6( ) c7(nk, ob, oa, U, nw, oc); #c1(ABHD12) c2(XP_011527516) c3(monoacylglycerol lipase ABHD12 isoform X2) c4(322337) c5( ) c6( ) c7(nk, ob, oa, U, nw, oc); #c1(ABHD12B) c2(NP_001193602) c3(abhydrolase domain-containing protein 128 isoform a) c4(82348) c5(82349, 82350, 82351, 82352, 82353, 82354) c6( ) c7(fq); #c1(ABHD12B) c2(NP_861535) c3(abhydrolase domain-containing protein (2B isoform c) c4(255664) c5(255665, 255666, 255667, 255668, 255669, 255670) c6( ) c7(fq); #c1(ABHD12B) c2(XP_005267413) c3(abhydrolase domain-containing protein 128 isoform X3) c4( ) c5(280972, 280973, 280974, 280975, 280976) c6( ) c7(fq); #c1(ABHD12B) c2(XP_011534776) c3(abhydrolase domain-containing protein (28 isoform X5) c4(331223) c5(331224) c6( ) c7(fq); #c1(ABHDIGA) c2(NP_001170986) c3(abhydrolase domain-containing protein 16A isoform b) c4(69648, 69649) c5(69650, 69651, 69652, 69653, 69654, 69655, 69656, 69657, 69658, 69659, 69660, 69661, 69662, 69663, 69664, 69665, 69666, 69667, 69668, 69669, 69670, 69671, 69672, 69673, 69674, 69675, 69676, 69677, 69678, 69679, 69680, 69681, 69682, 69683, 69684, 69685, 69686, 69687, 69688, 69689, 69690, 69691, 69692, 69693, 69694, 69695, 69696, 69697, 69698, 69699, 69700, 69701, 69702, 69703, 69704, 69705, 69706, 69707, 69708, 69709, 69710, 69711, 69712, 69713, 69714, 69715, 69716, 69717, 69718, 69719, 69720, 69721, 69722, 69723, 69724, 69725) c6( ) c7(If, y, iQ, od, eZ); #c1(ABHD16A) c2(NP_066983) c3(abhydrolase domain-containing protein 16A isoform a) c4(207966, 207967, 207968) c5(207969, 207970, 207971, 207972, 207973, 207974, 207975, 207976, 207977, 207978, 207979, 207980, 207981, 207982, 207983, 207984, 207985, 207986, 207987, 207988, 207989, 207990, 207991, 207992, 207993, 207994, 207995, 207996, 207997, 207998, 207999, 208000, 208001, 208002, 208003, 208004, 208005, 208006, 208007, 208008, 208009, 208010, 208011, 208012, 208013, 208014, 208015, 208016, 208017) c6( ) c7(If, y, iQ, od, eZ); #c1(ABHD17B) c2(NP_001020951) c3(alpha/beta hydrolase domain-containing protein 178 isoform 2 precursor) c4(29116) c5(29117, 29118, 29119, 29120, 29121, 29122, 29123, 29124) c6(29125, 29126) c7(oe); #c1(ABHD17B) c2(NP_057098) c3(alpha/beta hydrolase domain-containing protein (78 isoform 1 precursor) c4(188898) c5(188899, 188900, 188901, 188902, 188903, 188904, 188905, 188906) c6(188907, 188908) c7(oe); #c1(ABHD2) c2(XP_005254890) c3(abhydrolase domain-containing protein 2 isoform X1) c4(273369) c5(273370, 273371, 273372, 273373, 273374, 273375, 273376, 273377, 273378, 273379) c6( ) c7(of, eZ); #c1(ABHD5) c2(NP_057090) c3(1-acylglycerol-3-phosphate O-acyltransferase ABHD5) c4(188883) c5(188884, 188885, 188886, 188887, 188888, 188889) c6( ) c7(ok, d, ol, fc, oj, fn, oh, v, cP, du, oi, og, cd, dr); #c1(ABHD6) c2(NP_065727) c3(monoacylglycerol lipase ABHD6) c4(205077, 205078) c5(205079, 205080, 205081, 205082, 205083, 205084, 205085, 205086, 205087, 205088, 205089, 205090, 205091, 205092, 205093, 205094, 205095, 205096, 205097, 205098, 205099, 205100, 205101, 205102, 205103, 205104, 205105, 205106) c6( ) c7(bL, om, v); #c1(ABHD6) c2(XP_005265392) c3(monoacylglycerol lipase ABHDG isoform X2) c4(279525, 279526) c5( ) c6( ) c7(bL, om, v); #c1(AB1I) c2(NP_001012768) c3(abl interactor 1 isoform b) c4(25268, 25269) c5(25270, 25271, 25272, 25273, 25274, 25275, 25276, 25277, 25278, 25279, 25280, 25281) c6( ) c7(ck, ac, iZ, oo, aW, bt, aT, r, n, ey, ci, m, t, on, q); #c1(ABI1) c2(NP_001012769) c3(abl interactor 1 isoform c) c4(25282) c5(25283, 25284, 25285, 25286, 25287, 25288) c6( ) c7(ck, ac, iZ, oo, aW, bt, aT, r, n, ey, ci, m, t, on, q); #c1(ABI1) c2(NP_001012770) c3(abl interactor 1 isoform d) c4(25289) c5(25290, 25291, 25292, 25293, 25294, 25295) c6( ) c7(ck, ac, iZ, oo, aW, bt, aT, r, n, ey, ci, m, t, on, q); #c1(ABI1) c2(NP_001171587) c3(abl interactor 1 isoform e) c4(70656) c5(70657, 70658, 70659, 70660, 70661, 70662) c6( ) c7(ck, ac, iZ, oo, aW, bt, aT, r, n, ey, ci, m, t, on, q); #c1(ABI1) c2(NP_001171590) c3(abl interactor 1 isoform f) c4(70670) c5(70671, 70672, 70673, 70674, 70675, 70676) c6( ) c7(ck, ac, iZ, oo, aW, bt, aT, r, n, ey, ci, m, t, on, q); #c1(ABI1) c2(NP_001171591) c3(abl interactor 1 isoform g) c4(70677) c5(70678, 70679, 70680, 70681, 70682, 70683) c6( ) c7(ck, ac, iZ, oo, aW, bt, aT, r, n, ey, ci, m, t, on, q); #c1(ABI1) c2(NP_001171592) c3(abl interactor 1 isoform h) c4(70684) c5(70685, 70686, 70687, 70688, 70689, 70690) c6( ) c7(ck, ac, iZ, oo, aW, bt, aT, r, n, ey, ci, m, t, on, q); #c1(ABI1) c2(NP_001171593) c3(abl interactor 1 isoform i) c4(70691) c5(70692, 70693, 70694, 70695, 70696, 70697) c6( ) c7(ck, ac, iZ, oo, aW, bt, aT, r, n, ey, ci, m, t, on, q); #c1(ABI1) c2(NP_001171594) c3(abl interactor 1 isoform j) c4(70698) c5(70699, 70700, 70701, 70702, 70703, 70704) c6( ) c7(ck, ac, iZ, oo, aW, bt, aT, r, n, ey, ci, m, t, on, q); #c1(ABI1) c2(NP_001171596) c3(abl interactor 1 isoform 1) c4(70705) c5(70706, 70707, 70708, 70709, 70710, 70711) c6( ) c7(ck, ac, iZ, oo, aW, bt, aT, r, n, ey, ci, m, t, on, q); #c1(ABI1) c2(NP_005461) c3(abl interactor 1 isoform a) c4(156369, 156370) c5(156371, 156372, 156373, 156374, 156375, 156376, 156377, 156378, 156379, 156380, 156381, 156382) c6( ) c7(ck, ac, iZ, oo, aW, bt, aT, r, n, ey, ci, m, t, on, q); #c1(ABI1) c2(XP_005252390) c3(abl interactor 1 isoform X4) c4(271683) c5(271684, 271685, 271686, 271687, 271688, 271689) c6( ) c7(ck, ac, iZ, oo, aW, bt, aT, r, n, ey, ci, m, t, on, q); #c1(ABI2) c2(NP_001269854) c3(abl interactor 2 isoform a) c4(102955, 102956, 102957) c5(102958, 102959, 102960, 102961, 102962, 102963, 102964, 102965, 102966, 102967, 102968, 102969, 102970, 102971, 102972, 102973, 102974, 102975) c6( ) c7(dY, op, il); #c1(ABI2) c2(NP_001269855) c3(abl interactor 2 isoform b) c4(102976) c5(102977, 102978, 102979, 102980, 102981, 102982) c6( ) c7(dY, op, iU); #c1(ABI2) c2(NP_001269856) c3(abl interactor 2 isoform d) c4(102983) c5(102984, 102985, 102986, 102987, 102988, 102989) c6( ) c7(dY, op, il); #c1(ABI2) c2(NP_005750) c3(abl interactor 2 isoform c) c4(158989) c5(158990, 158991, 158992, 158993, 158994, 158995) c6( ) c7(dY, op, il); #c1(ABI2) c2(XP_005246282) c3(abl interactor 2 isoform X11) c4(267008, 267009) c5(267010, 267011, 267012, 267013, 267014, 267015, 267016, 267017, 267018, 267019, 267020, 267021) c6( ) c7(dY, op, iU); #c1(ABI3) c2(NP_001128658) c3(ABI gene family member 3 isoform 2) c4(50454) c5(50455, 50456, 50457, 50458, 50459, 50460) c6( ) c7(bE, T, bz, ci, nB); #c1(ABI3) c2(NP_057512) c3(ABI gene family member 3 isoform 1) c4(191193) c5(191194, 191195, 191196, 191197, 191198, 191199) c6( ) c7(bE, T, bz, ci, nB); #c1(ABI3BP) c2(NP_056244) c3(target of Nesh-SH3 precursor) c4(186683) c5(186684, 186685, 186686, 186687, 186688, 186689, 186690, 186691) c6(186692, 186693) c7(dP, av, oq, hd, T, cA, nB, aw, j, ci, bz); #c1(ABI3BP) c2(XP_005247340) c3(target of Nesh-SH3 isoform X4) c4(267812) c5(267813, 267814, 267815, 267816, 267817, 267818, 267819, 267820) c6(267821, 267822) c7(dP, av, oq, hd, T, cA, nB, aw, j, ci, bz); #c1(ABI3BP) c2(XP_005247366) c3(target of Nesh-SH3 isoform X50) c4(267823) c5(267824, 267825, 267826, 267827, 267828, 267829, 267830) c6(267831, 267832) c7(dP, av, oq, hd, T, cA, nB, aw, j, ci, bz); #c1(ABI3BP) c2(XP_011510949) c3(target of Nesh-SH3 isoform X22) c4( ) c5(301415) c6( ) c7(dP, av, oq, hd, T, cA, nB, aw, j, ci, bz); #c1(ABI3BP) c2(XP_011510959) c3(target of Nesh-SH3 isoform X44) c4(301416) c5(301417, 301418, 301419, 301420, 301421, 301422, 301423, 301424) c6(301425, 301426) c7(dP, av, oq, hd, T, cA, nB, aw, j, ci, bz); #c1(ABI3BP) c2(XP_011510961) c3(target of Nesh-SH3 isoform X46) c4( ) c5(301427) c6( ) c7(dP, av, oq, hd, T, cA, nB, aw, j, ci, bz); #c1(ABL1) c2(NP_005148) c3(tyrosine-protein kinase ABL1 isoform a) c4(153388, 153389, 153390) c5(153391, 153392, 153393, 153394, 153395, 153396, 153397, 153398, 153399, 153400, 153401, 153402, 153403, 153404, 153405, 153406, 153407, 153408) c6( ) c7(oO, T, os, fR, pf, fd, oJ, x, oB, pp, oG, K, hu, or, pk, oP, pr, aW, g, bw, ou, iT, oy, U, cj, k, m, gn, gp, ht, DE, n, oY, pn, eW, br, pe, oC, oN, h, hn, ox, pj, on, pi, oS, aq, po, pm, oU, bP, pg, ph, oM, fM, dA, oD, ol, v, pd, q, er, eA, oT, aT, dz, oF, an, ae, af, iU, oV, r, lo, hf, t, f, pb, ck, ov, pa, nM, iM, dT, oX, bt, ps, hl, ow, aD, oA, jh, OR, oz, pL, oH, ol, hF, ad, av, hd, fx, ot, oz, oW, hD, fb, pq, iL, d, je, et, kD, bz, ok, eU, pc, oQ, kS, iu); #c1(ABL1) c2(NP_009297) c3(tyrosine-protein kinase ABL1 isoform b) c4(171806, 171807) c5(171808, 171809, 171810, 171811, 171812, 171813, 171814, 171815, 171816, 171817, 171818, 171819) c6( ) c7(oO, T, os, fR, pf, fd, oJ, x, OB, pp, oG, K, hu, or, pk, oP, pr, aW, g, bw, ou, iT, ay, U, cj, k, m, gn, gp, ht, oE, n, oY, pn, eW, br, pe, oC, oN, h, hn, ox, pj, on, pi, oS, aq, po, pm, oU, bP, pg, ph, oM, fM, dA, oD, ol, v, pd, q, er, eA, oT, aT, dz, oF, an, ae, af, iU, oV, r, lo, hf, t, f, pb, ck, ov, pa, nM, iM, dT, oX, bt, ps, hl, ow, aD, oA, jh, oR, oZ, pl, OH, oL, hF, ad, av, hd, fx, ot, oz, oW, hD, fb, pq, iL, d, je, et, kD, bz, ok, eU, pc, ol, kS, iu); #c1(ABL2) c2(NP_001129472) c3(Abelson tyrosine-protein kinase 2 isoform d) c4(51540) c5(51541, 51542, 51543, 51544, 51545, 51546) c6( ) c7(T, j, ci, pz, ad, py, aT, h, K, jj, ae, af, iU, aW, ff, kp, en, px, pt, fq, ck, pv, pu, z, pw, bt); #c1(ABL2) c2(NP_001129473) c3(Abelson tyrosine-protein kinase 2 isoform e) c4(51547) c5(51548, 51549, 51550, 51551, 51552, 51553) c6( ) c7(T, j, ci, pz, ad, py, aT, h, K, jj, ae, af, iU, aW, ff, kp, en, px, pt, fq, ck, pv, pu, z, pw, bt); #c1(ABL2) c2(NP_001161708) c3(Abelson tyrosine-protein kinase 2 isoform f) c4(65835) c5(65836, 65837, 65838, 65839, 65840, 65841) c6( ) c7(T, j, ci, pz, ad, py, aT, h, K, jj, ae, af, iU, aW, ff, kp, en, px, pt, fq, ck, pv, pu, z, pw, bt); #c1(ABL2) c2(NP_001161709) c3(Abelson tyrosine-protein kinase 2 isoform g) c4(65842) c5(65843, 65844, 65845, 65846, 65847, 65848) c6( ) c7(T, j, ci, pz, ad, py, aT, h, K, jj, ae, af, iU, aW, ff, kp, en, px, pt, fq, ck, pv, pu, z, pw, bt); #c1(ABL2) c2(NP_001161710) c3(Abelson tyrosine-protein kinase 2 isoform h) c4(65849) c5(65850, 65851, 65852, 65853, 65854, 65855) c6( ) c7(T, j, ci, pz, ad, py, aT, h, K, jj, ae, af, iU, aW, ff, kp, en, px, pt, fq, ck, pv, pu, z, pw, bt); #c1(ABL2) c2(NP_001161711) c3(Abelson tyrosine-protein kinase 2 isoform i) c4(65856) c5(65857, 65858, 65859, 65860, 65861, 65862) c6( ) c7(T, j, ci, pz, ad, py, aT, h, K, jj, ae, af, iU, aW, ff, kp, en, px, pt, fq, ck, pv, pu, z, pw, bt); #c1(ABL2) c2(NP_005149) c3 (Abelson tyrosine-protein kinase 2 isoform c) c4(153409) c5(153410, 153411, 153412, 153413, 153414, 153415) c6( ) c7(T, j, ci, pz, ad, py, aT, h, K, jj, ae, af, iU, aW, ff, kp, en, px, pt, fq, ck, pv, pu, z, pw, bt); #c1(ABL2) c2(NP_009298) c3(Abelson tyrosine-protein kinase 2 isoform b) c4(171820) c5(171821, 171822, 171823, 171824, 171825, (71826) c6( ) c7(T, j, ci, pz, ad, py, aT, h, K, jj, ae, af, iU, aW, ff, kp, en, px, pt, fq, ck, pv, pu, z, pw, bt); #c1(ABL2) c2(XP_005245145) c3 (Abelson tyrosine-protein kinase 2 isoform X1) c4(266325) c5(266326, 266327, 266328, 266329, 266330, 266331) c6( ) c7(T, j, ci, pz, ad, py, aT, h, K, jj, ae, af, iU, aW, ff, kp, en, px, pt, fq, ck, pv, pu, z, pw, bt); #c1(ABL1MI) c2(NP_001003407) c3(actin-binding LIM protein 1 isoform b) c4(19823, 19824, 19825) c5(19826, 19827, 19828, 19829, 19830, 19831, 19832, 19833, 19834, 19835, 19836, 19837, 19838, 19839, 19840, 19841, 19842) c6( ) c7(bL, T, A, cz, j, y, lB); #c1(ABL1MI) c2(NP_001003408) c3(actin-binding LIM protein 1 isoform c) c4(19843) c5(19844, 19845, 19846, 19847, 19848, 19849, 19850, 19851) c6( ) c7(bL, T, A, cz, j, y, lB); #c1(ABL1MI) c2(NP_002304) c3(actin-binding LIM protein 1 isoform a) c4(127425, 127426, 127427) c5(127428, 127429, 127430, 127431, 127432, 127433, 127434, 127435, 127436, 127437, 127438, 127439, 127440) c6( ) c7(bL, T, A, cz, j, y, lB); #c1(ABL1MI) c2(NP_006711) c3(actin-binding LIM protein 1 isoform s) c4(167330, 167331) c5(167332, 167333, 167334, 167335, 167336, 167337, 167338, 167339, 167340, 167341) c6( ) c7(bL, T, A, cz, j, y, lB); #c1(ABL1MI) c2(XP_005269881) c3(actin-binding LIM protein 1 isoform X10) c4(282954) c5(282955, 282956, 282957, 282958, 282959, 282960) c6( ) c7(bL, T, A, cz, j, y, lB); #c1(ABL1MI) c2(XP_005269885) c3(actin-binding LIM protein 1 isoform X14) c4(282961) c5(282962, 282963, 282964, 282965, 282966) c6( ) c7(bL, T, A, cz, j, y, lB); #c1(ABL1MI) c2(XP_005269887) c3(actin-binding LIM protein 1 isoform X15) c4( ) c5(282967, 282968, 282969, 282970, 282971, 282972, 282973, 282974, 282975, 282976, 282977) c6( ) c7(bL, T, A, cz, j, y, lB); #c1(ABL1MI) c2(XP_006717901) c3(actin-binding LIM protein 1 isoform X4) c4(291386, 291387) c5( ) c6( ) c7(bL, T, A, cz, j, y, lB); #c1(ABL1MI) c2(XP_006717909) c3(actin-binding LIM protein 1 isoform X11) c4( ) c5(291388, 291389, 291390, 291391, 291392) c6( ) c7(bL, T, A, cz, j, y, lB); #c1(ABL1MI) c2(XP_011538103) c3(actin-binding LIM protein 1 isoform X1) c4(335668) c5(335669, 335670, 335671, 335672, 335673, 335674, 335675, 335676, 335677, 335678, 335679, 335680) c6( ) c7(bL, T, A, cz, j, y, lB); #c1(ABL1MI) c2(XP_011538105) c3(actin-binding LIM protein 1 isoform X5) c4( ) c5(335681, 335682, 335683, 335684, 335685, 335686) c6( ) c7(bL, T, A, cz, j, y, lB); #c1(ABL1MI) c2(XP_011538106) c3(actin-binding LIM protein 1 isoform X7) c4(335687) c5(335688) c6( ) c7(bL, T, A, cz, j, y, lB); #c1(ABL1MI) c2(XP_011538107) c3(actin-binding LIM protein 1 isoform X8) c4( ) c5(335689) c6( ) c7(bL, T, A, cz, j, y, lB); #c1(ABL1MI) c2(XP_011538108) c3(actin-binding LIM protein 1 isoform X9) c4(335690) c5(335691) c6( ) c7(bL, T, A, cz, j, y, lB); #c1(ABL1M2) c2(NP_001123555) c3(actin-binding LIM protein 2 isoform 1) c4(47537, 47538) c5(47539, 47540, 47541, 47542, 47543, 47544, 47545, 47546, 47547, 47548, 47549, 47550) c6( ) c7(j, ad); #c1(ABL1M2) c2(NP_001123556) c3(actin-binding LIM protein 2 isoform 2) c4(47551) c5(47552, 47553, 47554, 47555, 47556, 47557) c6( ) c7(j, ad); #c1(ABL1M2) c2(NP_001123557) c3(actin-binding LIM protein 2 isoform 3) c4(47558, 47559, 47560) c5(47561, 47562, 47563, 47564, 47565, 47566, 47567, 47568, 47569, 47570, 47571, 47572, 47573, 47574, 47575, 47576, 47577, 47578) c6( ) c7(j, ad); #c1(ABL1M2) c2(NP_001123558) c3(actin-binding LIM protein 2 isoform 4) c4(47579) c5(47580, 47581, 47582, 47583, 47584, 47585) c6( ) c7(j, ad); #c1(ABL1M2) c2(NP_001123559) c3(actin-binding LIM protein 2 isoform 5) c4(47586, 47587) c5(47588, 47589, 47590, 47591, 47592, 47593, 47594, 47595, 47596, 47597, 47598, 47599) c6( ) c7(j, ad); #c1(ABL1M2) c2(NP_001123560) c3(actin-binding LIM protein 2 isoform 7) c4(47600) c5(47601, 47602, 47603, 47604, 47605, 47606) c6( ) c7(j, ad); #c1(ABL1M2) c2(NP_001273617) c3(actin-binding LIM protein 2 isoform 8) c4(105449) c5(105450, 105451, 105452, 105453, 105454, 105455, 105456, 105457, 105458) c6( ) c7(j, ad); #c1(ABL1M2) c2(NP_115808) c3(actin-binding LIM protein 2 isoform 6) c4(224010) c5(224011, 224012, 224013, 224014, 224015, 224016) c6( ) c7(j, ad); #c1(ABL1M2) c2(XP_011511887) c3(actin-binding LIM protein 2 isoform X10) c4( ) c5(302603) c6( ) c7(j, ad); #c1(ABO) c2(NP_065202) c3(histo-blood group ABO system transferase) c4(204568) c5(204569, 204570, 204571, 204572, 204573, 204574) c6( ) c7(bM, T, qn, qo, aC, ea, e, fd, qj, ci, aE, pX, M, dV, K, eL, aa, dH, aW, hC, az, fc, kt, pD, aB, pl, bw, fT, qr, cj, m, eg, pJ, qm, dD, pG, pA, aw, aj, pM, pU, cd, qg, pN, ql, h, dL, pl, A, aq, mo, cA, lf, mg, pV, qq, it, qc, bL, pF, qd, pl, aA, cz, v, j, qf, J, jZ, qp, aT, qv, qk, dl, eu, qw, pR, en, t, qs, bN, ck, pH, pa, pl, ac, pZ, pS, dy, ao, hl, id, pY, qb, jN, eq, qi, hF, pW, ad, kL, hX, oz, qh, oW, hD, nJ, cH, cW, qt, ff, aF, pT, Z, bz, qa, qe, pE, ag, pP, gZ, dX, aS, qu, ei, pK, pB, y, pC); #c1(ABR) c2(NP_001153218) c3(active breakpoint cluster region-related protein isoform c) c4(60550) c5( ) c6( ) c7(jf, T, dD, qz, ea, mG, qx, hl, qy, aq, eq, K, aR, ad); #c1(ABR) c2(NP_001243776) c3(active breakpoint cluster region-related protein isoform d) c4(91696) c5(91697, 91698, 91699, 91700, 91701, 91702) c6( ) c7(jf, T, dD, qz, ea, mG, qx, hl, qy, aq, eq, K, aR, ad); #c1(ABR) c2(NP_001269078) c3(active breakpoint cluster region-related protein isoform e) c4(100389) c5(100390, 100391, 100392, 100393, 100394, 100395) c6( ) c7(jf, T, dD, qz, ea, mG, qx, hl, qy, aq, eq, K, aR, ad); #c1(ABR) c2(NP_068781) c3(active breakpoint cluster region-related protein isoform a) c4(210541) c5(210542, 210543, 210544, 210545, 210546, 210547, 210548, 210549, 210550, 210551, 210552, 210553, 210554) c6( ) c7(jf, T, dD, qz, ea, mG, qx, hl, qy, aq, eq, K, aR, ad); #c1(ABR) c2(XP_011522113) c3(active breakpoint cluster region-related protein isoform X1) c4( ) c5(316747, 316748, 316749, 316750, 316751) c6( ) c7(jf, T, dD, qz, ea, mG, qx, hl, qy, aq, eq, K, aR, ad); #c1(ABRA) c2(NP_631905) c3(actin-binding Rho-activating protein) c4(238282) c5(238283, 238284, 238285, 238286, 238287, 238288) c6( ) c7(W, fq, K, mx); #c1(ABT1) c2(NP_037507) c3(activator of basal transcription 1) c4(175910) c5(175911, 175912, 175913, 175914, 175915, 175916, 175917, 175918) c6( ) c7(eZ, aA); #c1(ACAA1) c2(NP_001123882) c3(3-ketoacyl-CoA thiolase, peroxisomal isoform b) c4(47955) c5(47956, 47957, 47958, 47959, 47960, 47961, 47962, 47963, 47964, 47965, 47966, 47967, 47968) c6( ) c7(bL, z, r, cS, aL, qA, bq, dF, q); #c1(ACAA1) c2(NP_001598) c3(3-ketoacyl-CoA thiolase, peroxisomal isoform a) c4(119025, 119026, 119027) c5(119028, 119029, 119030, 119031, 119032, 119033, 119034, 119035, 119036, 119037, 119038, 119039) c6( ) c7(bL, z, r, cS, aL, qA, bq, dF, q); #c1(ACAA1) c2(XP_006713186) c3(3-ketoacyl-CoA thiolase, peroxisomal isoform X3) c4( ) c5(287051, 287052, 287053, 287054, 287055) c6( ) c7(bL, z, r, cS, aL, qA, bq, dF, q); #c1(ACAA1) c2(XP_011531952) c3(3-ketoacyl-CoA thiolase, peroxisomal isoform X2) c4(327982) c5( ) c6( ) c7(bL, z, r, cS, aL, qA, bq, dF, q); #c1(ACAA2) c2(NP_006102) c3(3-ketoacyl-CoA thiolase, mitochondrial) c4(161894) c5(161895, 161896, 161897, 161898, 161899, 161900) c6( ) c7(bL, dF, qA); #c1(ACACA) c2(NP_942131) c3(acetyl-CoA carboxylase 1 isoform 1) c4(260494, 260495, 260496) c5(260497, 260498, 260499, 260500, 260501, 260502, 260503, 260504, 260505, 260506, 260507, 260508, 260509, 260510, 260511, 260512, 260513, 260514, 260515, 260516, 260517, 260518, 260519, 260520, 260521, 260522, 260523, 260524, 260525, 260526, 260527, 260528, 260529, 260530, 260531, 260532, 260533, 260534, 260535) c6( ) c7(bL, T, eR, aD, qB, fF, bB, n, cL, bS, j, ci, v, aY, o, ad, hX, qC, dP, qH, ft, ib, qK, aT, iv, jy, qG, dQ, qD, ae, qE, hm, iU, nO, cH, aW, fc, iw, cr, qL, eZ, fe, mv, t, fq, ck, qJ, ql, cO, bE, m, qF, i, B, hS); #c1(ACACA) c2(NP_942134) c3(acetyl-CoA carboxylase 1 isoform 3) c4(260536) c5(260537, 260538, 260539, 260540, 260541, 260542, 260543, 260544) c6( ) c7(bL, T, eR, aD, qB, fF, bB, n, cL, bS, j, ci, v, aY, o, ad, hX, qC, dP, qH, ft, ib, qK, aT, iv, jy, qG, dQ, qD, ae, qE, hm, iU, nO, cH, aW, fc, iw, cr, qL, eZ, fe, mv, t, fq, ck, qJ, ql, cO, bE, m, qF, i, B, hS); #c1(ACACA) c2(XP_006721916) c3(acetyl-CoA carboxylase 1 isoform X1) c4(294398, 294399) c5(294400, 294401) c6( ) c7(bL, T, eR, aD, qB, fF, bB, n, cL, bS, j, ci, v, aY, o, ad, hX, qC, dP, qH, ft, ib, qK, aT, iv, jy, qG, dQ, qD, ae, qE, hm, iU, nO, cH, aW, fc, iw, cr, qL, eZ, fe, mv, t, fq, ck, qJ, ql, cO, bE, m, qF, i, B, hS); #c1(ACACA) c2(XP_006725383) c3(acetyl-CoA carboxylase 1 isoform X2) c4(296804, 296805) c5( ) c6( ) c7(bL, T, eR, aD, qB, fF, bB, n, cL, bS, j, ci, v, aY, o, ad, hX, qC, dP, qH, ft, ib, qK, aT, iv, jy, qG, dQ, qD, ae, qE, hm, iU, nO, cH, aW, fc, iw, cr, qL, eZ, fe, mv, t, fq, ck, qJ, ql, cO, bE, m, qF, i, B, hS); #c1(ACACA) c2(XP_011523003) c3(acetyl-CoA carboxylase 1 isoform X3) c4(317462) c5( ) c6( ) c7(bL, T, eR, aD, qB, fF, bB, n, cL, bS, j, ci, v, aY, o, ad, hX, qC, dP, qH, ft, ib, qK, aT, iv, jy, qG, dQ, qD, ae, qE, hm, iU, n, cH, aW, fc, iw, cr, qL, eZ, fe, mv, t, fq, ck, qJ, ql, cO, bE, m, qF, i, B, hS); #c1(ACACA) c2(XP_011523005) c3 (acetyl-CoA carboxylase 1 isoform X4) c4(317463, 317464, 317465) c5(317466, 317467, 317468, 317469, 317470) c6( ) c7(bL, T, eR, aD, qB, fF, bB, n, cL, bS, j, ci, v, aY, o, ad, hX, qC, dP, qH, ft, ib, qK, aT, iv, jy, qG, dQ, qD, ae, qE, hm, iU, nO, cH, aW, fc, iw, cr, qL, eZ, fe, mv, t, fq, ck, qJ, ql, cO, bE, m, qF, i, B, hS); #c1(ACACA) c2(XP_011523006) c3(acetyl-CoA carboxylase 1 isoform X5) c4( ) c5(317471, 317472, 317473, 317474, 317475, 317476, 317477, 317478, 317479, 317480, 317481, 317482, 317483, 317484, 317485, 317486, 317487, 317488, 317489, 317490, 317491) c6( ) c7(bL, T, eR, aD, qB, fF, bB, n, cL, bS, j, ci, v, aY, o, ad, hX, qC, dP, qH, ft, ib, qK, aT, iv, jy, qG, dQ, qD, ae, qE, hm, iU, nO, cH, aW, fc, iw, cr, qL, eZ, fe, mv, t, fq, ck, qJ, ql, cO, bE, m, qF, i, B, hS); #c1(ACACB) c2(XP_011536564) c3(acetyl-CoA carboxylase 2 isoform X1) c4(334039, 334040, 334041) c5(334042, 334043, 334044, 334045, 334046, 334047, 334048, 334049, 334050, 334051) c6( ) c7(bL, cH, if, qK, fc, aj, v, dy, ci, qM, ei, dQ, qN, af, J); #c1(ACACB) c2(XP_011536565) c3(acetyl-CoA carboxylase 2 isoform X2) c4(334052, 334053) c5(334054, 334055, 334056, 334057, 334058, 334059) c6( ) c7(bL, cH, if, qK, fc, aj, v, dy, ci, qM, ei, de, qN, af, J); #c1(ACACB) c2(XP_011536566) c3(acetyl-CoA carboxylase 2 isoform X4) c4( ) c5(334060, 334061, 334062, 334063, 334064, 334065, 334066, 334067, 334068, 334069, 334070, 334071) c6( ) c7(bL, cH, if, qK, fc, aj, v, dy, ci, qM, ei, dQ, qN, af, J); #c1(ACACB) c2(XP_011536567) c3(acetyl-CoA carboxylase 2 isoform X5) c4( ) c5(334072, 334073, 334074, 334075, 334076, 334077) c6( ) c7(bL, cH, if, qK, fc, aj, v, dy, ci, qM, ei, dQ, qN, af, J); #c1(ACAD10) c2(NP_001130010) c3(acyl-CoA dehydrogenase family member 10 isoform a) c4(52419, 52420) c5(52421, 52422, 52423, 52424, 52425, 52426, 52427, 52428, 52429, 52430, 52431, 52432) c6( ) c7(bL, qO, ht, aT): #c1(ACAD10) c2(NP_079523) c3(acyl-CoA dehydrogenase family member 10 isoform b) c4(218576, 218577, 218578) c5(218579, 218580, 218581, 218582, 218583, 218584, 218585, 218586, 218587, 218588, 218589, 218590, 218591, 218592, 218593, 218594, 218595, 218596) c6( ) c7(bL, qO, ht, aT); #c1(ACAD8) c2(NP_055199) c3(isobutyryl-CoA dehydrogenase, mitochondrial) c4(179733, 179734, 179735) c5(179736, 179737, 179738, 179739, 179740, 179741, 179742, 179743, 179744, 179745, 179746, 179747, 179748, 179749, 179750, 179751, 179752) c6( ) c7(bL, T, qX, qP, qT, qQ, aA, aj, v, qW, ai, q, J, oz, ju, qV, qU, ji, K, qk, af, A, qZ, qS, qY, cr, r, fe, hp, iV, dr, bN, ag, gX, ra, y, qR); #c1(ACAD8) c2(XP_005271558) c3 (isobutyryl-CoA dehydrogenase, mitochondrial isoform X2) c4(283473) c5(283474, 283475, 283476, 283477, 283478, 283479) c6( ) c7(bL, T, qX, qP, qT, qQ, aA, aj, v, qW, ai, q, J, oz, ju, qV, qU, ji, K, qk, af, A, qZ, qS, qY, cr, r, fe, hp, iV, dr, bN, ag, gX, ra, y, qR); #c1(ACAD8) c2(XP_011541052) c3(isobutyryl-CoA dehydrogenase, mitochondrial isoform X1) c4( ) c5(339576) c6( ) c7(bL, T, qX, qP, qT, qQ, aA, aj, v, qW, ai, q, J, oz, ju, qV, qU, ji, K, qk, af, A, qZ, qS, qY, cr, r, fe, hp, iV, dr, bN, ag, gX, ra, y, qR); #c1(ACAD9) c2(NP_054768) c3(acyl-CoA dehydrogenase family member 9, mitochondrial) c4(177711) c5(177712, 177713, 177714, 177715, 177716, 177717, 177718, 177719, 177720, 177721, 177722, 177723, 177724) c6( ) c7(dd, cZ, aM, rb); #c1(ACAD9) c2(XP_011511044) c3(acyl-CoA dehydrogenase family member 9, mitochondrial isoform X1) c4(301467) c5( ) c6( ) c7(dd, cZ, aM, rb); #c1(ACADL) c2(NP_001599) c3(long-chain specific acyl-CoA dehydrogenase, mitochondrial precursor) c4(119040) c5(119041, 119042, 119043, 119044, 119045, 119046) c6( ) c7(bo, K); #c1(ACADS) c2(NP_000008) c3(short-chain specific acyl-CoA dehydrogenase, mitochondrial isoform 1 precursor) c4(22) c5(23, 24, 25, 26, 27, 28) c6( ) c7(bL, b, oj, bk, K, ei, i, rc); #c1(ACADSB) c2(NP_001600) c3(short/branched chain specific acyl-CoA dehydrogenase, mitochondrial precursor) c4(119047, 119048) c5(119049, 119050, 119051, 119052, 119053, 119054, 119055, 119056, 119057, (19058, 119059, 119060) c6( ) c7(aW, cz, aT, aj, re, dQ, rd, B, qa, bN); #c1(ACADVL) c2(NP_000009) c3(very long-chain specific acyl-CoA dehydrogenase, mitochondrial isoform 1 precursor) c4(29, 30) c5(31, 32, 33, 34, 35, 36, 37, 38, 39, 40, 41, 42, 43, 44, 45, 46) c6( ) c7(iv, cc, hl, rf); #c1(ACADVL) c2(NP_001029031) c3(very long-chain specific acyl-CoA dehydrogenase, mitochondrial isoform 2 precursor) c4(30736) c5(30737, 30738, 30739, 30740, 30741, 30742) c6( ) c7(iv, cc, hl, rf); #c1(ACADVL) c2(NP_001257376) c3 (very long-chain specific acyl-CoA dehydrogenase, mitochondrial isoform 3) c4(95268) c5(95269, 95270, 95271, 95272, 95273, 95274) c6( ) c7(iv, cc, hl, rf); #c1(ACAN) c2(NP_001126) c3(aggrecan core protein isoform 1 precursor) c4(49214) c5(49215, 49216, 49217, 49218, 49219, 49220, 49221, 49222) c6( ) c7(rt, oq, kh, dD, pG, ea, rr, eq, rm, rj, ai, rw, rn, rl, ld, ry, rC, rg, rD, rB, K, rE, rz, rv, af, A, rh, rs, bQ, pL, bN, rq, ru, ri, rx, ro, R, rF, y, rA, ik, rk, rp); #c1(ACAN) c2(NP_037359) c3 (aggrecan core protein isoform 2 precursor) c4(174979, (74980, 174981) c5(174982, 174983, 174984, 174985, 174986, 174987, 174988, 174989, 174990, 174991, 174992, 174993, 174994, 174995, 174996, 174997, 174998, 174999, 175000, 175001, 175002, 175003, 175004) c6( ) c7(rt, oq, kh, dD, pG, ea, rr, eq, rm, rj, ai, rw, rn, rl, ld, ry, rC, rg, rD, rB, K, rE, rz, rv, af, A, rh, rs, bl, pl, bN, rq, ru, ri, rx, ro, R, rF, y, rA, ik, rk, rp); #c1(ACAN) c2(XP_006720482) c3(aggrecan core protein isoform X1) c4( ) c5(293523) c6( ) c7(rt, oq, kh, dD, pG, ea, rr, eq, rm, rj, ai, rw, rn, rl, ld, ry, rC, rg, rD, rB, K, rE, rz, rv, af, A, rh, rs, bQ, pl, bN, rq, ru, ri, rx, ro, R, rF, y, rA, ik, rk, rp); #c1(ACAN) c2(XP_011519616) c3 (aggrecan core protein isoform X3) c4(314276) c5(314277, 314278, 314279, 314280, 314281, 314282, 314283, 314284) c6( ) c7(rt, oq, kh, dD, pG, ea, rr, eq, rm, rj, ai, rw, rn, rl, ld, ry, rC, rg, rD, rB, K, rE, rz, rv, af, A, ch, rs, bQ, pl, bN, rq, ru, ri, rx, ro, R, rF, y, rA, ik, rk, rp); #c1(ACAT1) c2(NP_000010) c3 (acetyl-CoA acetyltransferase, mitochondrial precursor) c4(47) c5(48, 49, 50, 51, 52, 53) c6( ) c7(T, dD, rG, eR, ea, rd, rl, cz, ah, ci, eq, rP, eA, ad, nV, rJ, aT, rN, rO, dQ, ae, af, rL, rM, cm, mO, ac, co, qA, rH, rk, bC, B); #c1(ACAT2) c2(NP_001290182) c3(acetyl-CoA acetyltransferase, cytosolic isoform 2) c4(115/07) c5(115108, 115109, 115110, 115/11, 115112, 115113) c6( ) c7(iu, l, u, cH, aq, v, qA, j, fr, iV, dQ, B, ad); #c1(ACAT2) c2(NP_005882) c3(acetyl-CoA acetyltransferase, cytosolic isoform 1) c4(160092) c5(160093, 160094, 160095, 160096, 160097, 160098) c6( ) c7(iu, l, u, cH, aq, v, qA, j, fr, iV, dQ, B, ad); #c1(ACBD3) c2(NP_073572) c3(Golgi resident protein GCP60) c4(212896) c5(212897, 212898, 212899, 212900, 212901, 212902) c6( ) c7(eV, iW, rR, eX); #c1(ACBD4) c2(NP_001129176) c3(acyl-CoA-binding domain-containing protein 4 isoform 1) c4(51108) c5(51109, 51110, 51111, 51112, 51113, 51114, 51115, 51116, 51117, 51118) c6( ) c7(ce); #c1(ACBD4) c2(NP_001129/79) c3(acyl-CoA-binding domain-containing protein 4 isoform 3) c4(51119) c5(51120, 51121, 51122, 51123, 51124, 51125, 51126) c6( ) c7(de); #c1(ACBD4) c2(NP_078998) c3(acyl-CoA-binding domain-containing protein 4 isoform 2) c4(216523) c5(216524, 216525, 216526, 216527, 216528, 216529) c6( ) c7(De); #c1(ACBD4) c2(XP_011523558) c3(acyl-CoA-binding domain-containing protein 4 isoform X2) c4( ) c5(318064) c6( ) c7(oe); #c1(ACBD5) c2(NP_001035938) c3(acyl-CoA-binding domain-containing protein 5 isoform 2) c4(33499) c5(33500) c6( ) c7(nx, hq); #c1(ACBD5) c2(NP_001258441) c3(acyl-CoA-binding domain-containing protein 5 isoform 3) c4( ) c5(96075, 96076, 96077, 96078, 96079, 96080) c6( ) c7(nx, hq); #c1(ACBD5) c2(NP_001288182) c3(acyl-CoA-binding domain-containing protein 5 isoform 4) c4(113682) c5(113683, 113684, 113685, 113686, 113687) c6( ) c7(nx, hq); #c1(ACBD5) c2(NP_001288183) c3(acyl-CoA-binding domain-containing protein 5 isoform 5) c4(113688) c5(113689, 113690, 113691, 113692, 113693) c6( ) c7(nx, hq); #c1(ACBD5) c2(NP_663736) c3(acyl-CoA-binding domain-containing protein 5 isoform 1) c4(241523) c5(241524, 241525, 241526, 241527, 241528, 241529) c6( ) c7(nx, hq); #c1(ACBD5) c2(XP_006717593) c3(acyl-CoA-binding domain-containing protein 5 isoform X2) c4(291217) c5(291218, 291219, 291220, 291221, 291222, 291223) c6( ) c7(nx, hq); #c1(ACBD5) c2(XP_006717594) c3(acyl-CoA-binding domain-containing protein 5 isoform X3) c4(291224, 29/225) c5(291226, 291227, 291228, 291229, 291230, 291231, 291232, 291233, 291234, 291235, 291236, 291237, 291238) c6( ) c7(nx, hq); #c1(ACBD5) c2(XP_006717598) c3(acyl-CoA-binding domain-containing protein 5 isoform X1) c4(291239) c5(291240, 291241, 291242, 291243, 291244) c6( ) c7(nx, hq); #c1(ACBD5) c2(XP_011518061) c3(acyl-CoA-binding domain-containing protein 5 isoform X5) c4( ) c5(312106) c6( ) c7(nx, hq); #c1(ACBD6) c2(NP_115736) c3(acyl-CoA-binding domain-containing protein 6) c4(223752) c5(223753, 223754, 223755, 223756, 223757, 223758) c6( ) c7(oe, nA); #c1(ACCS) c2(NP_115981) c3(1-aminocyclopropane-1-carboxylate synthase-like protein 1) c4(225071, 225072, 225073, 225074) c5(225075, 225076, 225077, 225078, 225079, 225080, 225081, 225082, 225083, 225084, 225085, 225086, 225087, 225088, 225089, 225090, 225091, 225092, 225093, 225094, 225095) c6( ) c7(T, rS, qK, ff, h, j, fr, en, hm); #c1(ACCS) c2(XP_005253227) c3(1-aminocyclopropane-1-carboxylate synthase-like protein 1 isoform X1) c4( ) c5(272287, 272288) c6( ) c7(T, rS, qK, ff, h, j, fr, en, hm); #c1(ACCS) c2(XP_011518708) c3(1-aminocyclopropane-1-carboxylate synthase-like protein 1 isoform X2) c4( ) c5(313127) c6( ) c7(T, rS, qK, ff, h, j, fr, en, hm); #c1(ACD) c2(NP_001075955) c3 (adrenocortical dysplasia protein homolog isoform 1) c4(37279) c5(37280, 37281, 37282, 37283, 37284, 37285) c6( ) c7(bL, sc, T, rV, aG, e, fr, M, aT, rX, h, aa, sb, Y, rU, rY, rZ, ac, rT, rW, cj, sa, B); #c1(ACD) c2(NP_001075956) c3(adrenocortical dysplasia protein homolog isoform 3) c4( ) c5(37286, 37287) c6( ) c7(bL, sc, T, rV, aG, e, fr, M, aT, rx, h, aa, sb, Y, rU, rY, rZ, ac, rT, rW, cj, sa, B); #c1(ACD) c2(NP_075065) c3(adrenocortical dysplasia protein homolog isoform 2) c4(213641) c5(213642, 213643, 213644, 213645, 213646, 213647) c6( ) c7(bL, sc, T, rV, aG, e, fr, M, aT, rX, h, aa, sb, Y, rU, rY, rZ, ac, rT, rW, cj, sa, B); #c1(ACE) c2(NP_000780) c3(angiotensin-converting enzyme isoform 1 precursor) c4(15665, 15666, 15667) c5(15668, 15669, 15670, 15671, 15672, 15673, 15674, 15675, 15676, 15677, 15678, 15679, 15680, 15681, 15682, 15683, 15684, 15685) c6( ) c7(uB, T, uK, ea, bO, ci, aY, ui, aE, rw, dV, hu, hH, aa, wp, uY, vs, tu, iA, wE, lS, vF, fy, uL, cE, sr, wJ, vE, vX, ht, sh, cQ, aj, ww, cd, tD, td, vM, sW, lG, ey, uc, mR, sp, vu, ge, sm, fe, hq, vU, se, vL, E, mT, jv, cz, aA, bX, wy, nS, q, oT, tL, vz, tF, cy, jj, sR, sX, dl, eu, bA, wr, su, bf, fO, iy, sV, lo, uU, pb, tl, tR, sk, cT, rR, vK, tW, ua, ac, ig, hr, tz, sx, bt, ao, gV, wi, tS, iW, uG, jh, sJ, mw, dG, uu, fu, sg, tO, ad, wD, tX, w, vJ, tT, oW, lQ, uN, wm, vP, qZ, ue, cH, cW, ff, tZ, tv, iV, bz, uP, ag, od, lC, lf, tJ, bM, vS, if, e, bB, cL, ur, ss, sD, ub, vV, ws, vh, fS, bc, UM, eG, up, dH, sP, hl, kt, pD, sz, mZ, vA, mx, vy, tV, tG, wx, dD, gU, wu, n, uE, md, sd, do, ec, eV, mV, hi, mU, A, fJ, ty, vo, sE, sf, jq, dC, cA, hl, tl, at, ut, eo, tm, fl, sZ, B, tq, vQ, ul, tf, dA, mr, wL, eX, fF, ti, w, v, V, gM, wN, rS, sy, sl, ld, ae, to, tN, hM, tn, tA, vT, vR, uh, sK, vk, ck, vW, gX, hO, sL, vi, dM, vG, uj, eq, od, vc, uD, sj, mu, d, wc, th, un, Z, dr, bF, D, gk, ll, wz, gZ, y, kS, sC, sH, pC, fR, vf, al, l, qj, gF, wa, tg, wM, bW, M, vg, ta, K, wt, wf, aB, g, ve, uC, uy, U, uo, vn, uf, uH, sQ, sw, aw, pM, eh, fK, tB, vC, vd, wb, uV, sq, gi, tx, wd, cM, ar, kT, W, uk, wl, wk, bo, mg, gg, lB, L, wj, ug, fG, oe, jx, wo, uZ, mS, j, vp, sl, il, uW, J, o, hb, nX, bq, kl, Y, r, wl, tu, en, wA, tj, sn, tY, hc, dh, tM, uS, cS, dy, fD, sY, aZ, uD, bS, ah, gb, vN, sB, dP, fU, sN, vB, tt, vl, tH, uX, lR, tb, vj, cr, si, iR, qa, dR, sG, ei, sT, vt, st, tQ, wHl, eb, sA, fw, aR, dY, il, qM, de, eN, tE, tw, hm, aW, eM, wK, wB, sU, sv, uJ, pw, vl, tp, wh, ct, qN, vY, ai, cc, wG, ql, h, jy, dL, ux, mY, db, he, vx, wn, wC, aq, mo, gG, dw, op, mX, tk, vD, sF, uw, vb, bm, uz, te, va, bD, um, bL, eR, dK, uv, sM, cR, tc, tC, C, so, ga, uT, qp, aT, vH, bK, an, af, uA, ak, s, wf, sS, wg, pZ, vZ, fP, wq, mf, ud, vq, uq, qb, aD, vr, vm, tK, mC, dZ, av, F, ot, oz, mW, sO, ul, ts, vw, jo, vO, uQ, eZ, aF, uF, eK, es, bE, pE, dJ, uR, aS, tr, be, pK, tP, we, gq, us); #c1(ACE) c2(NP_001171528) c3(angiotensin-converting enzyme isoform 3 precursor) c4(70458, 70459) c5(70460, 70461, 70462, 70463, 70464, 70465, 70466, 70467, 70468, 70469, 70470) c6( ) c7(uB, T, uk, ea, bO, ci, aY, ui, aE, rw, dV, hu, hH, aa, wp, uY, vs, tU, iA, wE, lS, vF, fy, ul, cE, sr, wJ, vE, vX, ht, sh, cd, aj, ww, cd, tD, td, vM, sW, lG, ey, uc, mR, sp, vu, ge, sm, fe, hq, vU, se, vL, E, mT, jv, cz, aA, bX, wy, nS, q, oT, tl, vz, tF, cy, jj, sR, sX, dl, eu, bA, wr, su, bf, fO, iy, sV, lo, ul, pb, tl, tR, sk, cT, rR, vK, tW, ua, ac, ig, hr, tz, sx, bt, ao, gV, wi, tS, iW, uG, jh, sJ, mw, dG, uu, fu, sg, tO, ad, wD, tX, www, vJ, tT, oW, lQ, uN, wm, vP, qZ, ue, cH, cW, ff, tZ, tv, iV, bz, uP, ag, od, lC, lf, tJ, bM, vS, if, e, bB, cL, ur, ss, sD, ub, vV, ws, vh, fS, bc, UM, eG, up, dH, sP, hl, kt, pD, sz, mZ, vA, mx, vy, tV, tG, wx, dD, gU, wu, n, uE, md, sd, do, ec, eV, mV, hi, mU, A, fJ, ty, vo, sE, sf, jq, dC, cA, hL, tl, at, ut, eo, tm, fl, sZ, B, tq, vl, ul, tf, dA, mr, wl, eX, fF, ti, vv, v, V, gM, wN, rS, sy, sl, ld, ae, to, tN, hM, tn, tA, vT, vR, uh, sK, vk, ck, vW, gX, hO, sL, vi, dM, vG, uj, eq, od, vc, uD, sj, mu, d, wc, th, un, Z, dr, bF, D, gk, ll, wz, gZ, y, kS, sC, sH, pC, fR, vf, al, l, qj, gF, wa, tg, wM, bW, M, vg, ta, K, wt, wf, aB, g, ve, uC, uy, U, uo, vn, uf, uH, sQ, sw, aw, pM, eh, fK, tB, vC, vd, wb, uV, sq, gi, tx, wd, cM, ar, kT, W, uk, wl, wk, bo, mg, gg, lB, L, wj, ug, fG, oe, jx, wo, uZ, mS, j, vp, sl, il, uW, J, o, hb, nX, bq, kl, Y, r, wl, tu, en, wA, tj, sn, tY, hc, dh, tM, uS, cS, dy, fD, sY, aZ, uD, bS, ah, gb, vN, sB, dP, fU, sN, vB, tt, vl, tH, uX, lR, tb, vj, cr, si, iR, qa, dR, sG, ei, sT, vt, st, tQ, wHl, eb, sA, fw, aR, dY, il, qM, dQ, eN, tE, tw, hm, aW, eM, wK, wB, sU, sv, uJ, pw, vl, tp, wh, ct, qN, vY, ai, cc, wG, ql, h, jy, dL, ux, mY, db, he, vx, wn, wC, aq, mo, gG, dw, op, mX, tk, vD, sf, uw, vb, bm, uz, te, va, bD, um, bL, eR, dk, uv, sM, cR, tc, tC, C, so, ga, uT, qp, aT, vH, bK, an, af, uA, ak, s, wf, sS, wg, pZ, vZ, fP, wq, mf, ud, vq, uq, qb, aD, vr, vm, tK, mC, dZ, av, F, ot, oz, mW, sO, ul, ts, vw, jo, vO, ul, eZ, af, uF, ek, es, bE, pE, dJ, uR, aS, tr, be, pk, tP, we, gq, us); #c1(ACE) c2(NP_690043) c3(angiotensin-converting enzyme isoform 2 precursor) c4(244807) c5(244808, 244809, 244810, 244811, 244812, 244813) c6( ) c7(uB, T, uK, ea, bO, ci, aY, ui, aE, rw, dV, hu, hH, aa, wp, uY, vs, tU, iA, wE, lS, vF, fy, ul, cE, sr, wJ, vE, vX, ht, sh, cQ, aj, ww, cd, tD, td, vM, sW, lG, ey, uc, mR, sp, vu, ge, sm, fe, hq, vU, se, vL, E, mT, jv, cz, aA, bX, wy, nS, q, oT, tl, vz tF, cy, jj, sR, sX, dl, eu, bA, wr, su, bf, fO, iy, sV, lo, uU, pb, tl, tR, sk, cT, rR, vK, tW, ua, ac, ig, hr, tz, sx, bt, ao, gV, wi, tS, iW, uG, jh, sJ, mw, dG, uu, fu, sg, tO, ad, wD, tX, www, vJ, tT, oW, lQ, uN, wm, vP, qZ, ue, cH, cW, ff, tZ, tv, iV, bz, uP, ag, od, lC, lf, tJ, bM, vS, if, e, bB, cL, ur, ss, sD, ub, vV, ws, vh, fS, bc, uM, eG, up, dH, sP, hl, kt, pD, sz, mZ, vA, mx, vy, tV, tG, wx, dD, gU, wu, n, uE, md, sd, do, ec, eV, mV, hi, mU, A, fJ, ty, vo, sE, sf, jq, dC, cA, hL, tl, at, ut, eo, tm, fl, sZ, B, tq, vQ, ul, tf, dA, mr, wL, ex, fF, ti, vv, v, V, gM, wN, rS, sy, sl, ld, ae, to, tN, hM, tn, tA, vT, vR, uh, sK, vk, ck, vW, gX, hO, sL, vi, dM, vG, uj, eq, od, vc, uD, sj, mu, d, wc, th, un, Z, dr, bF, D, gk, ll, wz, gZ, y, kS, sC, sH, pC, fR, vf, al, l, qj, gF, wa, tg, wM, bW, M, vg, ta, K, wt, wf, aB, g, ve, uC, uy, U, uo, vn, uf, uH, sQ, sw, aw, pM, eh, fK, tB, vC, vd, wb, uV, sq, gi, tx, wd, cM, ar, kT, W, uk, wl, wk, bo, mg, gg, lB, L, wj, ug, fG, oe, jx, wo, uZ, mS, j, vp, sl, il, uW, J, o, hb, nX, bq, kl, Y, r, wl, tu, en, wA, tj, sn, tY, hc, dh, tM, uS, cS, dy, fD, sY, aZ, uD, bS, ah, gb, vN, sB, dP, fU, sN, vB, tt, vl, tH, uX, lR, tb, vj, cr, si, iR, qa, dR, sG, ei, sT, vt, st, tQ, wH, eb, sA, fw, aR, dY, il, qM, dQ, eN, tE, tw, hm, aW, eM, wK, wB, sU, sv, uJ, pw, vl, tp, wh, ct, qN, vY, ai, cc, wG, ql, h, jy, dL, ux, mY, db, he, vx, wn, wC, aq, mo, gG, dw, op, mX, tk, vD, sF, uw, vb, bm, uz, te, va, bD, um, bL, eR, dk, uv, sM, cR, tc, tC, C, so, ga, uT, qp, aT, vH, bK, an, af, uA, ak, s, wf, sS, wg, pZ, vZ, fP, wq, mf, ud, vq, uq, qb, aD, vr, vm, tK, mC, dZ, av, F, ot, oz, mW, sO, ul, ts, vw, jo, vO, ul, eZ, aF, uF, ek, es, bE, pE, dJ, uR, aS, tr, be, pk, tP, we, gq, us); #c1(ACE) c2(XP_006721800) c3 (angiotensin-converting enzyme isoform X2) c4( ) c5(294278) c6( ) c7(uB, T, uk, ea, bO, ci, aY, ui, aE, rw, dV, hu, hH, aa, wp, uY, vs, tU, iA, wE, lS, vF, fy, ul, cE, sr, wJ, vE, vX, ht, sh, cl, aj, ww, cd, tD, td, vM, sW, lG, ey, uc, mR, sp, vu, ge, sm, fe, hq, vU, se, vL, E, mT, jv, cz, aA, bX, wy, nS, q, oT, tl, vz, tF, cy, jj, sR, sX, dl, eu, bA, wr, su, bf, fO, iy, sV, lo, uU, pb, tl, tR, sk, cT, rR, vK, tW, ua, ac, ig, hr, tz, sx, bt, ao, gV, wi, tS, iW, uG, jh, sJ, mw, dG, uu, fu, sg, tO, ad, wD, tX, w, vJ, tT, oW, lQ, uN, wm, vP, qZ, ue, cH, cW, ff, tZ, tv, iV, bz, uP, ag, od, lC, lf, tJ, bM, vS, if, e, bB, cL, ur, ss, sD, ub, vV, ws, vh, fS, bc, UM, eG, up, dH, sP, hl, kt, pD, sz, mZ, vA, mx, vy, tV, tG, wx, dD, gU, wu, n, uE, md, sd, do, ec, eV, mV, hi, mu, A, fJ, ty, vo, sE, sf, jq, dC, cA, hl, tl, at, ut, eo, tm, fl, sZ, B, tq, vQ, ul, tf, dA, mr, wL, eX, fF, ti, w, v, V, gM, wN, rS, sy, sl, ld, ae, to, tN, hM, tn, tA, vT, vR, uh, sk, vk, ck, vW, gX, hO, sL, vi, dM, vG, uj, eq, od, vc, uD, sj, mu, d, wc, th, un, Z, dr, bF, D, gk, ll, wz, gZ, y, kS, sC, sH, pC, fR, vf, al, l, qj, gF, wa, tg, wM, bW, M, vg, ta, K, wt, wf, aB, g, ve, uC, uy, U, uo, vn, uf, uH, sQ, sw, aw, pM, eh, fK, tB, vC, vd, wb, uV, sq, gi, tx, wd, cM, ar, kT, W, uk, wl, wk, bo, mg, gg, lB, L, wj, ug, fG, oe, jx, wo, uZ, mS, j, vp, sl, il, uW, J, o, hb, nX, bq, kl, Y, r, wl, tu, en, wA, tj, sn, tY, hc, dh, tM, uS, cS, dy, fD, sY, aZ, uD, bS, ah, gb, vN, sB, dP, fU, sN, vB, tt, vl, tH, uX, lR, tb, vj, cr, si, iR, qa, dR, sG, ei, sT, vt, st, tl, wH, eb, sA, fw, aR, dY, il, qM, de, eN, tE, tw, hm, aW, eM, wK, wB, sU, sv, uJ, pw, vl, tp, wh, ct, qN, vY, ai, cc, wG, ql, h, jy, dL, ux, mY, db, he, vx, wn, wC, aq, mo, gG, dw, op, mX, tk, vD, sf, uw, vb, bm, uz, te, va, bD, um, bL, eR, dk, uv, sM, cR, tc, tC, C, so, ga, uT, qp, aT, vH, bK, an, af, uA, ak, s, wf, sS, wg, pZ, vZ, fP, wq, mf, ud, vq, uq, qb, aD, vr, vm, tK, mC, dZ, av, F, ot, oz, mW, sO, ul, ts, vw, jo, vO, uQ, eZ, aF, uF, ek, es, bE, pE, dJ, uR, aS, tr, be, pk, tP, we, gq, us); #c1(ACE2) c2(NP_068576) c3(angiotensin-converting enzyme 2 precursor) c4(209554, 209555) c5(209556, 209557, 209558, 209559, 209560, 209561, 209562, 209563, 209564, 209565, 209566, 209567, 209568, 209569, 209570, 209571, 209572, 209573, 209574, 209575, 209576, 209577, 209578, 209579, 209580, 209581, 209582, 209583, 209584, 209585, 209586, 209587, 209588, 209589, 209590, 209591, 209592, 209593, 209594, 209595, 209596, 209597, 209598, 209599, 209600, 209601, 209602, 209603, 209604, 209605, 209606, 209607, 209608, 209609, 209610, 209611, 209612, 209613, 209614, 209615, 209616, 209617, 209618, 209619, 209620, 209621, 209622, 209623, 209624, 209625, 209626, 209627, 209628, 209629, 209630, 209631, 209632, 209633, 209634, 209635, 209636, 209637, 209638, 209639, 209640, 209641, 209642, 209643, 209644, 209645, 209646, 209647, 209648, 209649, 209650, 209651, 209652) c6(209653) c7(bL, eq, T, dD, wi, fR, mr, wJ, ea, fG, oe, e, aw, wl, cz, cR, tc, fF, dG, uu, aj, C, sl, j, v, vp, eh, cd, J, cc, aE, do, ws, sN, wG, sy, M, dV, wP, cy, us, vc, K, vB, bq, wt, bc, hm, aa, ul, A, wc, sP, hl, cr, fl, si, mo, dw, uC, bz, wU, qa, ek, wT, wl, eM, wO, ll, gZ, te, wR, aS, dy, fD, wV, hh, wS, jr, vQ); #c1(ACER2) c2(NP_001010887) c3(alkaline ceramidase 2) c4(23975) c5(23976, 23977, 23978, 23979, 23980, 23981) c6( ) c7(T); #c1(ACER2) c2(XP_011516160) c3(alkaline ceramidase 2 isoform X3) c4(309401) c5(309402, 309403, 309404, 309405, 309406, 309407) c6( ) c7(T); #c1(ACER3) c2(NP_001287882) c3(alkaline ceramidase 3 isoform b) c4(113125) c5(113126, 113127, 113128, 113129, 113130, 113131) c6( ) c7(Y); #c1(ACER3) c2(NP_001287884) c3(alkaline ceramidase 3 isoform c) c4( ) c5(113132, 113133, 113134, 113135, 113136, 113137, 113138, 113139, 113140, 113141, 113142) c6( ) c7(Y); #c1(ACER3) c2(NP_060837) c3(alkaline ceramidase 3 isoform a) c4(198059, 198060) c5(198061, 198062, 198063, 198064, 198065, 198066, 198067, 198068) c6( ) c7(Y): #c1(ACER3) c2(XP_011543454) c3(alkaline ceramidase 3 isoform X2) c4(342349, 342350) c5(342351, 342352, 342353, 342354, 342355) c6( ) c7(Y); #c1(ACHE) c2(NP_001269378) c3(acetylcholinesterase isoform 3 precursor) c4(101363) c5(101364, 101365, 101366, 101367, 101368, 101369, 101370, 101371, 101372, 101373, 101374) c6( ) c7(xo, al, bO, qj, aR, xm, xy, dV, K, H, xa, aW, as, cm, bw, xd, eM, U, m, R, xh, cZ, xn, xu, aw, n, fr, xr, xC, ke, ft, no, xc, dd, he, xf, wX, xi, xq, fl, fe, wZ, xe, hq, xA, ky, cA, dB, kz, xj, L, bL, wY, xk, j, eA, xx, mP, aT, cy, bq, an, af, jf, qw, hM, Y, xl, t, xw, wA, dS, ck, ac, xB, gT, bt, xb, ao, xt, ad, av, ot, rN, xp, xz, xs, eZ, xv, wW, es, gk, kq, xg, X); #c1(ACHE) c2(NP_001289550) c3(acetylcholinesterase isoform E4-E5 precursor) c4(114231, 114232) c5(114233, 114234, 114235, 114236, 114237, 114238, 114239, 114240, 114241, 114242, 114243, 114244, 114245, 114246, 114247, 114248) c6( ) c7(xo, al, bO, qj, aR, xm, xy, dV, K, H, xa, aW, as, cm, bw, xd, eM, U, m, R, xh, cZ, xn, xu, aw, n, fr, xr, xC, ke, ft, no, xc, dd, he, xf, wX, xi, xq, fQ, fe, wZ, xe, hq, xA, ky, cA, dB, kz, xj, L, bL, wY, xk, j, eA, xx, mP, aT, cy, bq, an, af, jf, qw, hM, Y, xl, t, xw, wA, dS, ck, ac, xB, gT, bt, xb, ao, xt, ad, av, ot, rN, xp, xz, xs, eZ, xv, wW, es, gk, kq, xg, X); #c1(ACHE) c2(NP_001289551) c3(acetylcholinesterase isoform E4-E6 precursor) c4(114249, 114250, 114251) c5(114252, 114253, 114254, 114255, 114256, 114257, 114258, 114259, 114260, 114261, 114262, 114263, 114264, 114265, 114266) c6( ) c7(xo, al, bO, qj, aR, xm, xy, dV, K, H, xa, aW, as, cm, bw, xd, eM, U, m, R, xh, cZ, xn, xu, aw, n, fr, xr, xC, ke, ft, no, xc, dd, he, xf, wX, xi, xq, fQ, fe, wZ, xe, hq, xA, ky, cA, dB, kz, xj, L, bL, wY, xk, j, eA, xx, mP, aT, cy, bq, an, af, jf, qw, hM, Y, xl, t, xw, wA, dS, ck, ac, xB, gT, bt, xb, ao, xt, ad, av, ot, rN, xp, xz, xs, eZ, xv, wW, es, gk, kq, xg, X); #c1(ACHE) c2(XP_011514527) c3 (acetylcholinesterase isoform X2) c4(307226) c5( ) c6( ) c7(xo, al, bO, qj, aR, xm, xy, dV, K, H, xa, aW, as, cm, bw, xd, eM, U, m, R, xh, cZ, xn, xu, aw, n, fr, xr, xC, ke, ft, no, xc, dd, he, xf, wX, xi, xq, fQ, fe, wZ, xe, hq, xA, ky, cA, dB, kz, xj, L, bL, wY, xk, j, eA, xx, mP, aT, cy, bq, an, af, jf, qw, hM, Y, xl, t, xw, wA, dS, ck, ac, xB, gT, bt, xb, ao, xt, ad, av, ot, rN, xp, xz, xs, eZ, xv, wW, es, gk, kq, xg, X); #c1(ACHE) c2(XP_011514528) c3(acetylcholinesterase isoform X3) c4( ) c5(307227, 307228, 307229, 307230, 307231, 307232) c6( ) c7(xo, al, bO, qj, aR, xm, xy, dV, K, H, xa, aW, as, cm, bw, xd, eM, U, m, R, xh, cZ, xn, xu, aw, n, fr, xr, xC, ke, ft, no, xc, dd, he, xf, wX, xi, xq, fl, fe, wZ, xe, hq, xA, ky, cA, dB, kz, xj, L, bL, wY, xk, j, eA, xx, mP, aT, cy, bq, an, af, jf, qw, hM, Y, xl, t, xw, wA, dS, ck, ac, xB, gT, bt, xb, ao, xt, ad, av, ot, rN, xp, xz, xs, eZ, xv, wW, es, gk, kq, xg, X); #c1(ACIN1) c2(NP_001158286) c3(apoptotic chromatin condensation inducer in the nucleus isoform 2) c4(63544) c5(63545, 63546, 63547, 63548, 63549, 63550) c6( ) c7(cm, oV, iu, g, iE); #c1(ACIN1) c2(NP_001158287) c3(apoptotic chromatin condensation inducer in the nucleus isoform 3) c4(63551) c5(63552, 63553, 63554, 63555, 63556, 63557) c6( ) c7(cm, oV, iu, g, iE); #c1(ACIN1) c2(NP_001158288) c3(apoptotic chromatin condensation inducer in the nucleus isoform 4) c4(63558) c5(63559, 63560, 63561, 63562, 63563, 63564) c6( ) c7(cm, oV, iu, g, iE); #c1(ACIN1) c2(NP_055792) c3(apoptotic chromatin condensation inducer in the nucleus isoform 1) c4(183533) c5(183534, 183535, 183536, 183537, 183538, 183539) c6( ) c7(cm, oV, iu, g, iE); #c1(ACIN1) c2(XP_005267475) c3(apoptotic chromatin condensation inducer in the nucleus isoform X1) c4(281004) c5(281005, 281006, 281007, 281008, 281009, 281010, 281011, 281012, 281013, 281014, 281015) c6( ) c7(cm, oV, iu, g, iE); #c1(ACKR1) c2(NP_001116423) c3(atypical chemokine receptor 1 isoform a) c4(43804, 43805) c5(43806, 43807, 43808, 43809, 43810, 43811, 43812, 43813, 43814, 43815, 43816, 43817) c6( ) c7(gV, bM, qb, xF, aw, qd, xM, gA, xJ, fu, V, eA, aR, eW, xE, xl, dV, aT, h, mL, lQ, bU, xL, hH, wt, aa, hG, aW, cr, fe, xH, wT, xK, ql, xD, dc, qu, dy, xG, fD, oQ, lf); #c1(ACKR1) c2(NP_002027) c3(atypical chemokine receptor 1 isoform b) c4(123434) c5(123435, 123436, 123437, 123438, 123439, 123440, 123441, 123442, 123443, 123444, 123445, 123446, 123447, 123448) c6( ) c7(gV, bM, qb, xF, aw, qd, xM, gA, xJ, fu, V, eA, aR, eW, xE, xl, dV, aT, h, mL, IQ, bu, xL, hH, wt, aa, hG, aW, cr, fe, xH, wT, xK, ql, xD, dc, qu, dy, xG, fD, oQ, lf); #c1(ACKR2) c2(NP_001287) c3(atypical chemokine receptor 2) c4(113358) c5( ) c6( ) c7(bM, xN, T, dD, xS, ea, iW, al, j, eq, ad, dP, x, F, kH, aT, qU, h, lQ, ae, xP, xQ, je, xR, iZ, aW, Y, fJ, eZ, dN, hp, t, ck, xT, eU, iM, z, xO, hk, cj, m, k, y, vi); #c1(ACKR3) c2(XP_005246155) c3 (atypical chemokine receptor 3 isoform X1) c4(266949, 266950) c5(266951, 266952, 266953, 266954, 266955, 266956, 266957, 266958, 266959, 266960, 266961, 266962) c6( ) c7(bM, T, ya, ea, fd, bB, ci, ye, xY, ip, x, bv, rJ, K, eL, yo, aW, eF, cm, iP, yd, eM, iA, cj, k, m, ym, dD, n, fr, cd, yn, ql, h, dL, yi, A, yc, aq, yq, jq, ok, yf, bP, hj, tl, B, bL, iH, yk, hU, cz, v, yp, q, eA, eJ, hb, xW, yg, aT, xV, bK, yb, yj, ae, xU, s, oV, Y, r, t, nC, iK, ck, nM, yl, dy, ha, eq, ad, av, yh, xX, d, eH, eZ, iR, bz, dR, es, xZ, bE, y); #c1(ACKR4) c2(NP_848540) c3(atypical chemokine receptor 4) c4(253785, 253786) c5(253787, 253788, 253789, 253790, 253791, 253792, 253793, 253794, 253795, 253796, 253797, 253798) c6( ) c7(xS, yr, eU, iM, je, xR, xT, xO, Y, aW, ha, nl, nh, aT, dN, fJ, h, lQ, xP); #c1(ACLY) c2(NP_001290203) c3(ATP-citrate synthase isoform 3) c4(115146) c5(115147, 115148, 115149, 115150, 115151, 115152, 115153, 115154) c6( ) c7(av, T, iu, eM, fc, r, v, ci, K, q, bz, B); #c1(ACLY) c2(NP_001290204) c3(ATP-citrate synthase isoform 4) c4(115155) c5(115156, 115157, 115158, 115159, 115160, 115161, 115162, 115163, 115164, 115165, 115166, 115167, 115168, 115169, 115170, 115/71) c6( ) c7(av, T, iu, eM, fc, r, v, ci, K, q, bz, B); #c1(ACLY) c2(NP_942127) c3(ATP-citrate synthase isoform 2) c4(260480) c5( ) c6( ) c7(av, T, iu, eM, fc, r, v, ci, K, q, bz, B); #c1(ACLY) c2(XP_005257452) c3(ATP-citrate synthase isoform X1) c4(275127) c5(275128, 275129, 275130, 275131, 275132, 275133, 275134, 275135) c6( ) c7(av, T, iu, eM, fc, r, v, ci, K, q, bz, B); #c1(ACMSD) c2(NP_612199) c3(2-amino-3-carboxymuconate-6-semialdehyde decarboxylase) c4(236182) c5(236183, 236184, 236185, 236186, 236187, 236188) c6( ) c7(ys, ha, aq, an, B, aR); #c1(AC01) c2(NP_002188) c3(cytoplasmic aconitate hydratase) c4(126402, 126403, 126404, 126405) c5(126406, 126407, 126408, 126409, 126410, 126411, 126412, 126413, 126414, 126415, 126416, 126417, 126418, 126419, 126420, 126421, 126422, 126423, 126424, 126425, 126426, 126427, 126428, 126429, 126430, 126431, 126432, 126433) c6( ) c7(T, py, yw, af, yt, hk, Y, yx, aw, r, yy, yu, cd, j, f, q, ad, yv); #c1(AC02) c2(NP_001089) c3(aconitate hydratase, mitochondrial precursor) c4(38324, 38325, 38326, 38327) c5(38328, 38329, 38330, 38331, 38332, 38333, 38334, 38335, 38336, 38337, 38338, 38339, 38340, 38341, 38342, 38343, 38344, 38345, 38346, 38347, 38348, 38349, 38350, 38351) c6( ) c7(B, yz); #c1(ACOT1) c2(NP_001032238) c3(acyl-coenzyme A thioesterase 1) c4(31118) c5(31119, 31120, 31121, 31122, 31123) c6( ) c7(h, aq, if, qN, bq); #c1(ACOT13) c2(NP_001153566) c3(acyl-coenzyme A thioesterase 13 isoform 2) c4(60650) c5(60651, 60652, 60653, 60654, 60655, 60656) c6( ) c7(aj, yA, J, aT); #c1(ACOT13) c2(NP_060943) c3(acyl-coenzyme A thioesterase 13 isoform 1) c4(198833) c5(198834, 198835, 198836, 198837, 198838, 198839) c6( ) c7(aj, yA, J, aT); #c1(ACOT2) c2(NP_006812) c3(acyl-coenzyme A thioesterase 2, mitochondrial) c4(168299) c5(168300, 168301, 168302, 168303, 168304, 168305, 168306) c6( ) c7(bL, j, bq); #c1(ACOT9) c2(NP_001028755) c3(acyl-coenzyme A thioesterase 9, mitochondrial isoform b precursor) c4(30641) c5(30642, 30643, 30644, 30645, 30646, 30647, 30648) c6( ) c7(fc); #c1(ACOT9) c2(NP_001032248) c3(acyl-coenzyme A thioesterase 9, mitochondrial isoform a precursor) c4(31168) c5(31169, 31170, 31171, 31172, 31173, 31174) c6( ) c7(fc); #c1(ACOT9) c2(XP_005274529) c3(acyl-coenzyme A thioesterase 9, mitochondrial isoform X2) c4(285122) c5(285123, 285124, 285125, 285126, 285127) c6( ) c7(fc); #c1(ACOX1) c2(NP_001171968) c3(peroxisomal acyl-coenzyme A oxidase 1 isoform c) c4(72104) c5(72105, 72106, 72107, 72108, 72109) c6( ) c7(yD, fc, U, yB, K, cP, yC, B, bq); #c1(ACOX1) c2(NP_004026) c3(peroxisomal acyl-coenzyme A oxidase 1 isoform a) c4(142930) c5(142931, 142932, 142933, 142934, 142935, 142936, 142937, 142938, 142939, 142940, 142941) c6( ) c7(yD, fc, U, yB, K, cP, yC, B, bq); #c1(ACOX1) c2(NP_009223) c3(peroxisomal acyl-coenzyme A oxidase 1 isoform b) c4(171570) c5(171571, 171572, 171573, 171574, 171575, (71576) c6( ) c7(yD, fc, U, yB, K, cP, yC, B, bq); #c1(ACOX1) c2(XP_011523170) c3(peroxisomal acyl-coenzyme A oxidase 1 isoform X1) c4(317704) c5(317705, 317706, 317707, 317708, 317709, 317710) c6( ) c7(yD, fc, U, yB, K, cP, yC, B, bq); #c1(ACOX2) c2(NP_003491) c3(peroxisomal acyl-coenzyme A oxidase 2) c4(138740, 138741) c5(138742, 138743, 138744, 138745, 138746, 138747, 138748, 138749, 138750, 138751) c6( ) c7(mL, e, B, K); #c1(ACOX2) c2(XP_011532449) c3 (peroxisomal acyl-coenzyme A oxidase 2 isoform X2) c4( ) c5(328618, 328619) c6( ) c7(ml, e, B, K); #c1(ACOX3) c2(NP_001095137) c3(peroxisomal acyl-coenzyme A oxidase 3 isoform b) c4(40808) c5(40809, 40810, 40811, 40812, 40813, 40814) c6( ) c7(cF, ae, ad); #c1(ACOX3) c2(NP_003492) c3(peroxisomal acyl-coenzyme A oxidase 3 isoform a) c4(138752) c5(138753, 138754, 138755, 138756, 138757, 138758, 138759, 138760, 138761, 138762) c6( ) c7(cF, ae, ad); #c1(ACOXL) c2(NP_001136279) c3(acyl-coenzyme A oxidase-like protein) c4(54798, 54799) c5(54800, 54801, 54802, 54803, 54804, 54805, 54806, 54807, 54808, 54809, 54810, 54811) c6( ) c7(ey, aw, cj, ad, pN); #c1(ACOXL) c2(XP_011509712) c3(acyl-coenzyme A oxidase-like protein isoform X5) c4(300285) c5( ) c6( ) c7(ey, aw, cj, ad, pN); #c1(ACOXL) c2(XP_011509717) c3(acyl-coenzyme A oxidase-like protein isoform X10) c4( ) c5(300286) c6( ) c7(By, aw, cj, ad, pN); #c1(ACOXL) c2(XP_011509718) c3(acyl-coenzyme A oxidase-like protein isoform X11) c4(300287) c5(300288, 300289, 300290, 300291, 300292) c6( ) c7(ey, aw, cj, ad, pN); #c1(ACOXL) c2(XP_011509737) c3(acyl-coenzyme A oxidase-like protein isoform X25) c4(300293) c5(300294, 300295, 300296, 300297, 300298, 300299, 300300, 300301, 300302, 300303, 300304, 300305) c6( ) c7(ey, aw, cj, ad, pN); #c1(ACPI) c2(NP_001035739) c3(low molecular weight phosphotyrosine protein phosphatase isoform d) c4(33011, 33012) c5(33013, 33014, 33015, 33016, 33017, 33018, 33019, 33020, 33021, 33022, 33023) c6( ) c7(tJ, T, bM, yS, e, yY, fd, bB, cL, qj, ci, yM, aE, fm, dE, dV, K, aW, az, yK, mQ, kt, g, pl, cm, yX, yl, eM, vA, cj, m, yJ, yG, aj, ko, fr, hw, h, yT, yZ, bl, hg, A, fe, op, yR, yN, yP, mg, fL, bD, B, bL, aA, hz, v, j, y, yV, yU, V, q, o, J, eA, mj, nv, aT, dz, ae, iU, Y, yL, r, en, cl, iK, pb, ck, cT, yW, gX, mf, yH, OH, hF, mC, ad, kL, dP, py, yQ, hD, bj, IQ, pq, yF, d, lR, et, ff, cr, eZ, aF, iV, dr, qa, es, yE, bE, qu, ei, y, X, pC); #c1(ACPI) c2(NP_004291) c3(low molecular weight phosphotyrosine protein phosphatase isoform c) c4(144894) c5(144895, 144896, 144897, 144898, 144899, 144900) c6( ) c7(tJ, T, bM, yS, e, yY, fd, bB, cL, qj, ci, yM, aE, fm, dE, dV, K, aW, az, yK, mQ, kt, g, pl, cm, yX, yl, eM, vA, cj, m, yJ, yG, aj, ko, fr, hw, h, yT, yZ, bl, hg, A, fe, op, yR, yN, yP, mg, fL, bD, B, bL, aA, hz, v, j, yO, yV, yU, V, q, o, J, eA, mj, nv, aT, dz, ae, iU, Y, yL, r, en, cl, iK, pb, ck, cT, yW, gX, mf, yH, OH, hF, mC, ad, kL, dP, py, yQ, hD, bj, lQ, pq, yF, d, lR, et, ff, cr, eZ, aF, iV, dr, qa, es, yE, bE, qu, ei, y, X, pC); #c1(ACPI) c2(NP_009030) c3(low molecular weight phosphotyrosine protein phosphatase isoform b) c4(170318) c5(170319, 170320, 170321, 170322, 170323, 170324, 170325) c6( ) c7(tJ, T, bM, yS, e, yY, fd, bB, cl, qj, ci, yM, aE, fm, dE, dV, K, aW, az, yK, md, kt, g, pl, cm, yX, yl, eM, vA, cj, m, yJ, yG, aj, ko, fr, hw, h, yT, yZ, bl, hg, A, fe, op, yR, yN, yP, mg, fL, bD, B, bL, aA, hz, v, j, yO, yV, yU, V, q, o, J, eA, mj, nv, aT, dz, ae, iU, Y, yL, r, en, cl, iK, pb, ck, cT, yW, gX, mf, yH, OH, hF, mC, ad, kL, dP, py, yQ, hD, bj, lQ, pq, yF, d, lR, et, ff, cr, eZ, aF, iV, dr, qa, es, yE, bE, qu, ei, y, X, pC); #c1(ACP2) c2(NP_001289419) c3(lysosomal acid phosphatase isoform 4 precursor) c4(114185) c5(114186, 114187, 114188, 114189, 114190, 114191) c6( ) c7(ck, za, dA, pc, cj, ab, aT, ji, bt, ad); #c1(ACP2) c2(NP_001289420) c3(lysosomal acid phosphatase isoform 5 precursor) [4( ) c5(114192, 114193, 114194, 114195, 114196, 114197, 114198) c6( ) c7(ck, za, dA, pc, cj, ab, aT, ji, bt, ad); #c1(ACP2) c2(NP_001289421) c3(lysosomal acid phosphatase isoform 6) c4(114199) c5(114200, 114201, 114202, 114203, 114204) c6( ) c7(ck, za, dA, pc, cj, ab, aT, ji, bt, ad); #c1(ACP2) c2(NP_001601) c3(lysosomal acid phosphatase isoform 1 precursor) c4(119061, 119062, 119063) c5(119064, 119065, 119066, 119067, 119068, 119069, 119070, 119071, 119072, 119073, 119074, 119075, 119076) c6( ) c7(ck, za, dA, pc, cj, ab, aT, ji, bt, ad); #c1(ACP5) c2(NP_001104506) c3(tartrate-resistant acid phosphatase type 5 precursor) c4(42133) c5(42134, 42135, 42136, 42137, 42138, 42139, 42140, 42141, 42142, 42143, 42144, 42145, 42146, 42147, 42148, 42149, 42150, 42151) c6( ) c7(T, zm, zs, zt, iW, aA, zj, fd, v, zh, jE, ze, fr, zd, zo, ad, ai, zn, ft, ib, xE, zi, aE, zw, zf, dV, aT, zg, zp, bq, zu, ae, hi, d, kj, Y, zb, vj, eH, ff, zq, aF, en, pL, t, ve, bz, eF, vU, zr, yE, zl, zv, zc, k, eg, zk, B, y, vQ); #c1(ACP6) c2(XP_011507903) c3(lysophosphatidic acid phosphatase type 6 isoform X1) c4(298170) c5(298/71, 298172, 298/73, 298174, 298175, 298176, 298177, 298178) c6(298179, 298180) c7(dY); #c1(ACPP) c2(NP_001090) c3(prostatic acid phosphatase isoform PAP precursor) c4(38352) c5(38353, 38354, 38355, 38356, 38357, 38358) c6( ) c7(T, dA, iW, aA, cz, bB, aj, v, zx, zF, cd, ad, ai, eA, b, i, aT, h, zy, ae, d, zH, aW, zD, r, zC, S, uF, g, zE, bw, zz, ck, bE, cj, wq, dT, zG, m, zl, zB, bt, zA, ao); #c1(ACPP) c2(NP_001127666) c3(prostatic acid phosphatase isoform TM-PAP precursor) c4(49223) c5(49224, 49225, 49226, 49227, 49228, 49229, 49230) c6( ) c7(T, dA, iW, aA, cz, bB, aj, v, zx, zF, cd, ad, ai, eA, b, i, aT, h, zy, ae, d, zH, aW, zD, r, zC, S, uF, g, zE, bw, zz, ck, bE, cj, wq, dT, zG, m, zl, zB, bt, zA, ao); #c1(ACR) c2(NP_001088) c3(acrosin precursor) c4(38317) c5(38318, 38319, 38320, 38321, 38322, 38323) c6( ) c7(ck, iU, oW, eZ, jh, oY, cl, dy, S, fL, pb, y, bt, mC); #c1(ACRBP) c2(NP_115878) c3(acrosin-binding protein precursor) c4(224432) c5(224433, 224434, 224435, 224436, 224437, 224438, 224439, 224440) c6(224441, 224442) c7(T, bB, m, bu, t, ad); #c1(ACRC) c2(XP_011529367) c3(acidic repeat-containing protein isoform X1) c4(324746) c5( ) c6( ) c7(all); #c1(ACRC) c2(XP_011529369) c3(acidic repeat-containing protein isoform X4) c4( ) c5(324747, 324748, 324749, 324750, 324751, 324752, 324753, 324754, 324755, 324756) c6( ) c7(aU); #c1(ACRV1) c2(NP_001603) c3(acrosomal protein SP-10 isoform a precursor) c4(119077, 119078) c5(119079, 119080, 119081, 119082, 119083, 119084) c6( ) c7(wc); #c1(ACRV1) c2(NP_064454) c3(acrosomal protein SP-10 isoform b precursor) c4(202554, 202555) c5(202556, 202557, 202558, 202559, 202560, 202561, 202562, 202563, 202564, 202565, 202566, 202567, 202568, 202569, 202570, 202571) c6( ) c7(wc); #c1(ACRV1) c2(NP_064492) c3(acrosomal protein SP-10 isoform c precursor) c4(202580, 202581) c5(202582, 202583, 202584, 202585, 202586, 202587, 202588, 202589, 202590, 202591, 202592, 202593, 202594, 202595, 202596, 202597) c6( ) c7(wc); #c1(ACRV1) c2(NP_064493) c3(acrosomal protein SP-10 isoform d precursor) c4(202598, 202599, 202600, 202601) c5(202602, 202603, 202604, 202605, 202606, 202607, 202608, 202609, 202610, 202611, 202612, 202613, 202614, 202615, 202616, 202617, 202618, 202619, 202620, 202621, 202622, 202623, 202624, 202625, 202626, 202627, 202628, 202629, 202630, 202631, 202632, 202633, 202634, 202635) c6( ) c7(wc); #c1(ACSBG1) c2(NP_055977) c3(long-chain-fatty-acid-CoA ligase ACSBG1 isoform 1) c4(184850) c5(184851, 184852, 184853, 184854, 184855, 184856) c6( ) c7(dA, jh, eW, eA, zn, F, zK, aT, hH, dF, aW, lo, zL, zM, bz, zJ, bE, U, cj, pw, im, y, B); #c1(ACSBG1) c2(XP_011519691) c3(long-chain-fatty-acid-CoA ligase ACSBG1 isoform X1) c4(314369) c5( ) c6( ) c7(dA, jh, eW, eA, zn, F, zK, aT, hH, dF, aW, lo, zL, zM, bz, zJ, bE, U, cj, pw, im, y, B); #c1(ACSBG1) c2(XP_011519693) c3(long-chain-fatty-acid-CoA ligase ACSBG1 isoform X3) c4( ) c5(314370, 314371, 314372, 314373, 314374, 314375) c6( ) c7(dA, jh, eW, eA, zn, F, zK, aT, hH, dF, aW, lo, zL, zM, bz, zJ, bE, U, cj, pw, im, y, B); #c1(ACSBG2) c2(NP_001276107) c3(long-chain-fatty-acid-CoA ligase ACSBG2 isoform a) c4(108011) c5(108012, 108013, 108014, 108015, 108016, 108017) c6( ) c7(dF, gU); #c1(ACSF3) c2(NP_777577) c3(acyl-CoA synthetase family member 3, mitochondrial isoform 1 precursor) c4(251223) c5(251224, 251225, 251226, 251227, 251228, 251229, 251230, 251231, 251232, 251233) c6( ) c7(zN, zO, dq, fM, jh); #c1(ACSF3) c2(XP_011521245) c3(acyl-CoA synthetase family member 3, mitochondrial isoform X1) c4(316155) c5( ) c6( ) c7(zN, zO, dq, fM, jh); #c1(ACSF3) c2(XP_011521246) c3(acyl-CoA synthetase family member 3, mitochondrial isoform X2) c4( ) c5(316156, 316157, 316158, 316159, 316160, 316161) c6( ) c7(zN, zO, dq, fM, jh); #c1(ACSL1) c2(NP_001273640) c3(long-chain-fatty-acid-CoA ligase 1 isoform c) c4(105519) c5(105520, 105521, 105522, 105523, 105524, 105525) c6( ) c7(zQ, ac, uT, fO, zR, aj, v, ey, K, ci, bq, ei, zP, J); #c1(ACSL1) c2(NP_001273641) c3(long-chain-fatty-acid-CoA ligase 1 isoform d) c4(105526) c5( ) c6( ) c7(zQ, ac, uT, fO, zR, aj, v, ey, K, ci, bq, ei, zP, J); #c1(ACSL1) c2(XP_011530044) c3(long-chain-fatty-acid--CoA ligase 1 isoform X1) c4(325822) c5(325823, 325824, 325825, 325826, 325827, 325828, 325829, 325830, 325831, 325832, 325833, 325834, 325835, 325836, 325837, 325838, 325839, 325840, 325841, 325842, 325843, 325844, 325845, 325846) c6( ) c7(zQ, ac, uT, fO, zR, aj, v, ey, K, ci, bq, ei, zP, J); #c1(ACSL3) c2(NP_976251) c3(long-chain-fatty-acid-CoA ligase 3) c4(263070, 263071, 263072) c5(263073, 263074, 263075, 263076, 263077, 263078, 263079, 263080, 263081, 263082, 263083, 263084, 263085, 263086, 263087, 263088, 263089, 263090, 263091, 263092, 263093, 263094) c6( ) c7(zT, ae, bM, rV, dQ, zS, jx, aq, j, zV, bz, pM, zU, eA, zW, fq, B, ad); #c1(ACSL5) c2(NP_057318) c3(long-chain-fatty-acid-CoA ligase 5 isoform a) c4(190085) c5(190086, 190087, 190088, 190089, 190090, 190091, 190092, 190093, 190094, 190095) c6( ) c7(T, zY, s, zX, fO, eZ, r, ci, fl, g, dQ, eA, q); #c1(ACSL5) c2(NP_976314) c3(long-chain-fatty-acid-CoA ligase 5 isoform b) c4(263103, 263104, 263105) c5(263106, 263107, 263108, 263109, 263110, 263111, 263112, 263113, 263114, 263115, 263116, 263117, 263118, 263119, 263120, 263121, 263122, 263123, 263124, 263125, 263126, 263127) c6( ) c7(T, zY, s, zX, fO, eZ, r, ci, fl, g, dQ, eA, q); #c1(ACSL6) c2(NP_001009185) c3(long-chain-fatty-acid-CoA ligase 6 isoform b) c4(23564, 23565) c5(23566, 23567, 23568, 23569, 23570, 23571, 23572) c6( ) c7(iH, zZ, il, pm, bq, he, xa, ad); #c1(ACSL6) c2(NP_001192177) c3(long-chain-fatty-acid-CoA ligase 6 isoform d) c4( ) c5(81959, 81960, 81961, 81962, 81963, 81964, 81965, 81966, 81967, 81968, 81969, 81970, 81971) c6( ) c7(iH, zZ, il, pm, bq, he, xa, ad); #c1(ACSL6) c2(NP_001192179) c3(long-chain-fatty-acid-CoA ligase 6 isoform e) c4(81972, 81973, 81974) c5(81975, 81976, 81977, 81978, 81979, 81980, 81981, 81982, 81983, 81984, 81985, 81986, 81987, 81988, 81989, 81990, 81991, 81992, 81993, 81994, 81995, 81996, 81997) c6( ) c7(iH, zZ, il, pm, bq, he, xa, ad); #c1(ACSL6) c2(NP_001192180) c3(long-chain-fatty-acid-CoA ligase 6 isoform f) c4(81998) c5(81999, 82000, 82001, 82002, 82003, 82004) c6( ) c7(iH, zZ, il, pm, bq, he, xa, ad); #c1(ACSL6) c2(NP_056071) c3(long-chain-fatty-acid-CoA ligase 6 isoform a) c4(185506, 185507, 185508) c5(185509, 185510, 185511, 185512, 185513, 185514, 185515, 185516, 185517) c6( ) c7(iH, zZ, il, pm, bq, he, xa, ad); #c1(ACSM1) c2(XP_005255141) c3(acyl-coenzyme A synthetase ACSM1, mitochondrial isoform X1) c4(273550, 273551) c5(273552, 273553, 273554, 273555, 273556, 273557, 273558, 273559, 273560, 273561, 273562) c6( ) c7(qa, av, ci, ei, cz); #c1(ACSM1) c2(XP_011544032) c3(acyl-coenzyme A synthetase ACSM1, mitochondrial isoform X5) c4(343197) c5(343198, 343199, 343200, 343201, 343202, 343203) c6( ) c7(qa, av, ci, ei, cz); #c1(ACSM1) c2(XP_011544034) c3(acyl-coenzyme A synthetase ACSM1, mitochondrial isoform X7) c4( ) c5(343204, 343205, 343206, 343207, 343208) c6( ) c7(qa, av, ci, ei, cz); #c1(ACSM2B) c2(NP_001098539) c3(acyl-coenzyme A synthetase ACSM2B, mitochondrial) c4(41350, 41351) c5(41352, 41353, 41354, 41355, 41356, 41357) c6( ) c7(om); #c1(ACSM2B) c2(XP_005255361) c3(acyl-coenzyme A synthetase ACSM2B, mitochondrial isoform X2) c4( ) c5(273614, 273615, 273616, 273617, 273618, 273619, 273620, 273621, 273622, 273623, 273624, 273625) c6( ) c7(om); #c1(ACSM3) c2(NP_005613) c3(acyl-coenzyme A synthetase ACSM3, mitochondrial isoform 1) c4(157942) c5(157943, 157944, 157945, 157946, 157947, 157948) c6( ) c7(qa, T, ac, eR, oe, wf, cz, v, aS, Aa, dy, ei, dQ, nS, uC, ad, bJ); #c1(ACSM3) c2(NP_973729) c3(acyl-coenzyme A synthetase ACSM3, mitochondrial isoform 2) c4(262694) c5(262695, 262696, 262697, 262698, 262699, 262700) c6( ) c7(qa, T, ac, eR, oe, wf, cz, v, aS, Aa, dy, ei, dl, nS, uC, ad, bJ); #c1(ACSM3) c2(XP_011544213) c3(acyl-coenzyme A synthetase ACSM3, mitochondrial isoform X1) c4(343343) c5(343344, 343345, 343346, 343347, 343348, 343349) c6( ) c7(qa, T, ac, eR, oe, wf, cz, v, aS, Aa, dy, ei, dQ, nS, uC, ad, bJ); #c1(ACSS1) c2(NP_001239605) c3(acetyl-coenzyme A synthetase 2-like, mitochondrial isoform 3) c4(88569) c5(88570, 88571, 88572, 88573, 88574, 88575, 88576, 88577, 88578, 88579, 88580, 88581) c6( ) c7(dy, ac); #c1(ACSS1) c2(NP_001239606) c3(acetyl-coenzyme A synthetase 2-like, mitochondrial isoform 4 precursor) c4(88582) c5(88583, 88584, 88585, 88586, 88587, 88588) c6( ) c7(dy, ac); #c1(ACSS1) c2(NP_115890) c3(acetyl-coenzyme A synthetase 2-like, mitochondrial isoform 1 precursor) c4(224540, 224541, 224542) c5(224543, 224544, 224545, 224546, 224547, 224548) c6( ) c7(dy, ac); #c1(ACSS1) c2(XP_006723722) c3(acetyl-coenzyme A synthetase 2-like, mitochondrial isoform X1) c4(295306) c5(295307, 295308, 295309, 295310, 295311, 295312, 295313, 295314, 295315, 295316, 295317, 295318) c6( ) c7(dy, ac); #c1(ACSS2) c2(NP_001070020) c3(acetyl-coenzyme A synthetase, cytoplasmic isoform 2) c4(34580) c5(34581, 34582, 34583, 34584, 34585, 34586) c6( ) c7(bL, T, rS, h, fr, j, B, hm); #c1(ACSS2) c2(NP_001229322) c3 (acetyl-coenzyme A synthetase, cytoplasmic isoform 3) c4(84199) c5(84200, 84201, 84202, 84203, 84204, 84205) c6( ) c7(bL, T, rS, h, fr, j, B, hm); #c1(ACSS2) c2(NP_061147) c3(acetyl-coenzyme A synthetase, cytoplasmic isoform 1) c4(199545, 199546, 199547) c5(199548, 199549, 199550, 199551, 199552, 199553, 199554, 199555, 199556, 199557, 199558, 199559, 199560) c6( ) c7(bL, T, rS, h, fr, j, B, hm); #c1(ACSS2) c2(XP_011527211) c3(acetyl-coenzyme A synthetase, cytoplasmic isoform X7) c4( ) c5(321755, 321756) c6( ) c7(bL, T, rS, h, fr, j, B, hm); #c1(ACSS2) c2(XP_011527213) c3(acetyl-coenzyme A synthetase, cytoplasmic isoform X9) c4( ) c5(321757, 321758, 321759, 321760, 321761, 321762) c6( ) c7(bL, T, rS, h, fr, j, B, hm); #c1(ACSS3) c2(NP_078836) c3(acyl-CoA synthetase short-chain family member 3, mitochondrial precursor) c4(215654) c5(215655, 215656, 215657, 215658, 215659, 215660) c6( ) c7(fq, dy); #c1(ACSS3) c2(XP_005269207) c3(acyl-CoA synthetase short-chain family member 3, mitochondrial isoform X1) c4(282484) c5(282485) c6( ) c7(fq, dy); #c1(ACSS3) c2(XP_011537042) c3(acyl-CoA synthetase short-chain family member 3, mitochondrial isoform X3) c4( ) c5(334825, 334826, 334827, 334828, 334829) c6( ) c7(fq, dy); #c1(ACTA1) c2(NP_001091) c3(actin, alpha skeletal muscle) c4( ) c5( ) c6( ) c7(bM, dD, Ah, if, Am, aw, aj, eq, qN, Ac, Aj, cc, no, Ae, Ag, vJ, il, oj, Ak, vc, bl, rz, af, Af, Ad, An, Ab, Ai, Al); #c1(ACTA2) c2(XP_011538318) c3(actin, aortic smooth muscle isoform X1) c4(335996, 335997, 335998) c5(335999, 336000, 336001, 336002, 336003, 336004, 336005, 336006, 336007, 336008, 336009, 336010, 336011, 336012, 336013, 336014, 336015, 336016, 336017, 336018, 336019, 336020, 336021, 336022, 336023, 336024) c6( ) c7(T, Ap, vl, Am, al, Aq, Av, j, fr, bd, Ao, dP, av, iu, cc, M, vw, cy, vc, At, rz, bf, As, aJ, hl, Au, g, eF, ac, Ar, gY, mx, aS, dy, be, vF, X); #c1(ACTB) c2(NP_001092) c3(actin, cytoplasmic 1) c4(39786, 39787, 39788, 39789, 39790, 39791, 39792, 39793) c5( ) c6( ) c7(bM, T, pf, al, bB, ci, zW, ip, x, aE, Al, aU, eL, li, zQ, AP, aW, Az, g, as, bw, eM, AM, vA, mx, iS, k, wu, ht, nA, zP, eW, tD, co, ft, Ax, h, dL, ey, AT, AD, fe, op, kn, iO, bP, sZ, AN, B, AB, fM, dA, AL, AS, Aw, aA, v, j, AE, il, AD, q, eJ, AK, aT, kf, AJ, bK, ae, kl, s, iU, Y, r, lo, en, f, AA, mG, AH, hr, AU, AC, gT, bt, vi, yv, jh, Ay, ad, dP, av, yt, AD, ds, qh, ab, ji, wm, AF, ff, aF, bz, dr, bF, gk, bE, AR, gZ, bC, ei, hE, AG); #c1(ACTBL2) c2(NP_001017992) c3(beta-actin-like protein 2) c4(26739) c5(26740, 26741, 26742, 26743, 26744, 26745) c6( ) c7(bL, bM, T, aw, ct, eA, ad, qH, AW, sO, gd, aT, dV, iv, bK, an, af, A, aW, cm, AV, at, eg, qc); #c1(ACTC1) c2(NP_005150) c3(actin, alpha cardiac muscle 1 proprotein) c4(153416, 153417) c5(153418, 153419, 153420, 153421, 153422, 153423, 153424, 153425, 153426, 153427, 153428, 153429) c6( ) c7(AY, oe, Am, aA, cz, j, cd, cc, Bc, vc, Ba, K, jo, rz, Bb, AX, mo, AZ, cL, gY, mx, Be, bD, Bd); #c1(ACTG1) c2(NP_001605) c3(actin, cytoplasmic 2) c4(119085, 119086, 119087) c5(119088, 119089, 119090, 119091, 119092, 119093, 119094, 119095, 119096, 119097, 119098, 119099, 119100, 119101, 119102, 119103, 119104, 119105, 119106, 119107, 119108, 119109, 119110, 119111, 119112, 119113, 119114, 119115, 119116) c6( ) c7(bL, bM, T, aw, j, ct, nA, ci, zW, zP, eA, qH, x, AW, AD, sO, Bj, gd, aT, dV, iv, Bi, h, bK, an, af, bA, A, zQ, Bf, aW, Az, cm, Bg, ck, kn, dc, AV, mG, at, k, eg, Bh); #c1(ACTG2) c2(NP_001186822) c3(actin, gamma-enteric smooth muscle isoform 2 precursor) c4(78686) c5(78687, 78688, 78689, 78690, 78691, 78692) c6( ) c7(bL, bM, T, Bl, Bk, aw, Bm, ct, fu, eA, qH, AW, hW, sO, gd, Bn, aT, dV, iv, bK, an, af, A, aW, Bo, hJ, uC, AV, at, eg, X); #c1(ACTG2) c2(NP_001606) c3(actin, gamma-enteric smooth muscle isoform 1 precursor) c4(119117) c5(119118, 119119, 119120, 119121, 119122, 119123, 119124, 119125, 119126, 119127) c6( ) c7(bL, bM, T, BI, Bk, aw, Bm, ct, fu, eA, qH, AW, hW, sO, gd, Bn, aT, dV, iv, bK, an, af, A, aW, Bo, hJ, uC, AV, at, eg, X); #c1(ACTL6A) c2(NP_004292) c3(actin-like protein GA isoform 1) c4(144901) c5(144902, 144903, 144904, 144905, 144906, 144907, 144908, 144909, 144910) c6( ) c7(bv, T, aF, Bp, aA): #c1(ACTL6A) c2(NP_817 (26) c3(actin-like protein 6A isoform 2) c4(253121, 253122) c5(253123, 253124, 253125, 253126, 253127, 253128, 253129, 253130, 253131, 253132, 253133, 253134, 253135) c6( ) c7(bv, T, aF, Bp, aA); #c1(ACTL6B) c2(NP_057272) c3(actin-like protein 6B) c4(189783) c5(189784, 189785, 189786, 189787, 189788, 189789, 189790, 189791) c6(189792, 189793) c7(Bp); #c1(ACTL8) c2(XP_011540513) c3(actin-like protein 8 isoform X2) c4(338987) c5(338988) c6( ) c7(cd, bX); #c1(ACTL9) c2(NP_848620) c3(actin- like protein 9) c4(254001) c5(254002, 254003, 254004, 254005, 254006, 254007) c6( ) c7(eE); #c1(ACTN1) c2(NP_001093) c3(alpha-actinin-1 isoform b) c4(40559) c5( ) c6( ) c7(dP, Br, dX, j, K, cl, y, Bq); #c1(ACTN1) c2(NP_001123476) c3(alpha-actinin-1 isoform a) c4(47121) c5(47122, 47123, 47/24, 47125, 47126, 47127) c6( ) c7(dP, Br, dX, j, K, cl, y, Bq); #c1(ACTN1) c2(NP_001123477) c3(alpha-actinin-1 isoform c) c4(47128) c5(47129, 47130, 47131, 47132, 47133, 47134) c6( ) c7(dP, Br, dX, j, K, cl, y, Bq); #c1(ACTN2) c2(NP_001094) c3(alpha-actinin-2 isoform 1) c4(40786) c5( ) c6( ) c7(AY, cc, Bs, mx, vc, bS, bl, he, Bt); #c1(ACTN2) c2(NP_001265272) c3(alpha-actinin-2 isoform 2) c4(98106) c5(98107, 98108, 98109, 98110, 98111, 98112, 98113) c6( ) c7(AY, cc, Bs, mx, vc, bS, bl, he, Bt); #c1(ACTN2) c2(NP_001265273) c3(alpha-actinin-2 isoform 3) c4(98114, 98115) c5(98116, 98117, 98118, 98119, 98120, 98121, 98122, 98123, 98124, 98125, 98126) c6( ) c7(AY, cc, Bs, mx, vc, bS, bl, he, Bt); #c1(ACTN3) c2(NP_001095) c3(alpha-actinin-3 isoform 1) c4(40966, 40967) c5( ) c6( ) c7(Bv, sF, lb, Bw, m, t, Bu, af); #c1(ACTN3) c2(NP_001245300) c3(alpha-actinin-3 isoform 2) c4(93032) c5(93033, 93034) c6( ) c7(Bv, sF, lb, Bw, m, t, Bu, af); #c1(ACTN4) c2(NP_004915) c3(alpha-actinin-4) c4(150729, 150730) c5(150731, 150732, 150733, 150734, 150735, 150736, 150737, 150738, 150739, 150740, 150741, 150742, 150743, 150744, 150745, 150746, 150747, 150748, 150749, 150750, 150751, 150752, 150753, 150754) c6( ) c7(T, dA, eR, dk, cz, n, ah, ci, lu, zP, aY, ye, q, cd, uW, er, dP, BB, ft, aE, aT, vw, h, By, dL, Bz, wt, eL, A, zQ, qt, aW, Y, gi, r, g, t, cm, bz, Bx, sk, vW, go, dy, m, ul, iu, BA); #c1(ACTN4) c2(XP_005259338) c3 (alpha-actinin-4 isoform X2) c4(276067) c5( ) c6( ) c7(T, dA, eR, dk, cz, n, ah, ci, lu, zP, aY, ye, q, cd, uW, er, dP, BB, ft, aE, aT, vw, h, By, dL, Bz, wt, eL, A, zQ, qt, aW, Y, gi, r, g, t, cm, bz, Bx, sk, vW, go, dy, m, ul, iu, BA); #c1(ACTN4) c2(XP_006723469) c3(alpha-actinin-4 isoform X1) c4(295221) c5( ) c6( ) c7(T, dA, eR, dk, cz, n, ah, ci, lu, zP, aY, ye, q, cd, uW, er, dP, BB, ft, aE, aT, vw, h, By, dL, Bz, wt, eL, A, zQ, qt, aW, Y, gi, r, g, t, cm, bz, Bx, sk, vW, go, dy, m, ul, iu, BA); #c1(ACTR1A) c2(NP_005727) c3(alpha-centractin) c4(158896, 158897, 158898) c5(158899, 158900, 158901, 158902, 158903, 158904, 158905, 158906, 158907, 158908, 158909, 158910, 158911, 158912, 158913, 158914, 158915, 158916) c6( ) c7(ck, BC, BD, ib, oV, fd, aq, hF, B); #c1(ACTR1B) c2(NP_005726) c3(beta-centractin) c4(158889) c5(158890, 158891, 158892, 158893, 158894, 158895) c6( ) c7(dL, B, ac); #c1(ACTR2) c2(NP_001005386) c3(actin-related protein 2 isoform a) c4(21603, 21604, 21605) c5(21606, 21607, 21608, 21609, 21610, 21611, 21612) c6( ) c7(T, iQ, dA, aE, r, aA, h, By, lo, wR, BF, ey, BE, BG, t, ae, q, ad); #c1(ACTR2) c2(NP_005713) c3(actin-related protein 2 isoform b) c4(158780) c5(158781, 158782, 158783, 158784, 158785, 158786, 158787, 158788, 158789, 158790, 158791, 158792, 158793, 158794, 158795, 158796, 158797) c6( ) c7(T, iQ, dA, aE, r, aA, h, By, lo, wR, BF, ey, BE, BG, t, ae, q, ad); #c1(ACTR3) c2(NP_001264069) c3(actin-related protein 3 isoform 2) c4(97475) c5(97476, 97477, 97478, 97479, 97480, 97481, 97482, 97483, 97484, 97485, 97486, 97487, 97488, 97489, 97490, 97491, 97492) c6( ) c7(h, t, BG); #c1(ACTR3) c2(NP_005712) c3(actin-related protein 3 isoform 1) c4(158770, 158771, 158772) c5(158773, 158774, 158775, 158776, 158777, 158778, 158779) c6( ) c7(h, t, BG); #c1(ACTR3B) c2(NP_001035225) c3(actin-related protein 3B isoform 2) c4(32638, 32639) c5(32640, 32641, 32642, 32643, 32644, 32645) c6( ) c7(aw, cd, aF, bK, aA); #c1(ACTR3B) c2(NP_065178) c3 (actin-related protein 38 isoform 1) c4(204469, 204470) c5(204471, 204472, 204473, 204474, 204475, 204476, 204477, 204478, 204479, 204480, 204481, 204482, 204483, 204484, 204485, 204486) c6( ) c7(aw, cd, aF, bK, aA); #c1(ACTR3B) c2(XP_006716128) c3(actin-related protein 38 isoform X1) c4( ) c5(290088) c6( ) c7(aw, cd, aF, bK, aA); #c1(ACTR3B) c2(XP_011514729) c3(actin-related protein 3B isoform X6) c4( ) c5(307385) c6( ) c7(aw, cd, aF, bK, aA); #c1(ACTR5) c2(NP_079131) c3(actin-related protein 5) c4(217194) c5(217195, 217196, 217197, 217198, 217199, 217200) c6( ) c7(c); #c1(ACTRT1) c2(NP_612146) c3(actin-related protein T1) c4(236057) c5(236058, 236059, 236060, 236061, 236062, 236063) c6( ) c7(h, ea, Y, bz); #c1(ACVR1) c2(NP_001096) c3 (activin receptor type-1 precursor) c4(4/277, 41278, 41279, 41280, 41281) c5( ) c6( ) c7(bM, T, kh, aC, BH, ci, BM, q, ad, eA, BI, cF, BJ, aT, dz, ey, rz, ae, kl, et, ff, r, en, as, t, dr, sn, ac, BL, gY, w, BK, m, bD, us); #c1(ACVR1B) c2(NP_004293) c3(activin receptor type-18 isoform a precursor) c4(144911, 144912, 144913, 144914) c5(144915, 144916, 144917, 144918, 144919, 144920, 144921, 144922, 144923, 144924, 144925, 144926, 144927, 144928, 144929, 144930, 144931, 144932, 144933) c6( ) c7(Bl, hd, BN, aE, nB, aW, hR, hy, j, qF, ne); #c1(ACVR1B) c2(NP_064732) c3(activin receptor type-18 isoform b) c4( ) c5(203566, 203567, 203568, 203569, 203570) c6( ) c7(Bl, hd, BN, aE, nB, aW, hR, hy, j, qF, ne); #c1(ACVR1B) c2(NP_064733) c3(activin receptor type-18 isoform c precursor) c4(203571) c5(203572, 203573, 203574, 203575, 203576, 203577) c6( ) c7(Bl, hd, BN, aE, nB, aW, hR, hy, j, qF, ne); #c1(ACVR1C) c2(NP_001104501) c3(activin receptor type-1C isoform 2) c4( ) c5(42114, 42115, 42116, 42117, 42118) c6( ) c7(t, v, B, m, aq); #c1(ACVR1C) c2(NP_001104502) c3(activin receptor type-1C isoform 3 precursor) c4(42119) c5(42120, 42121, 42122, 42123, 42124, 42125) c6( ) c7(t, v, B, m, aq); #c1(ACVR1C) c2(NP_001104503) c3(activin receptor type-1C isoform 4 precursor) c4(42126) c5(42127, 42128, 42129, 42130, 42131, 42132) c6( ) c7(t, v, B, m, aq); #c1(ACVR1C) c2(NP_660302) c3(activin receptor type-1C isoform 1 precursor) c4(240799, 240800) c5(240801, 240802, 240803, 240804, 240805, 240806, 240807) c6( ) c7(t, v, B, m, aq); #c1(ACVR2A) c2(NP_001265508) c3(activin receptor type-2A isoform 1 precursor) c4(98715) c5(98716, 98717, 98718, 98719, 98720, 98721, 98722, 98723, 98724, 98725, 98726) c6( ) c7(T, wu, aZ, eb, bB, q, ad, aE, oW, aT, wm, cp, kl, BO, vj, r, uC, bE, BP, gX, m, X); #c1(ACVR2A) c2(NP_001265509) c3(activin receptor type-2A isoform 2) c4(98727) c5(98728, 98729, 98730, 98731, 98732) c6( ) c7(T, wu, aZ, eb, bB, q, ad, aE, oW, aT, wm, cp, kl, BO, vj, r, uC, bE, BP, gX, m, X); #c1(ACVR2B) c2(NP_001097) c3(activin receptor type-28 precursor) c4(41282) c5(41283, 41284, 41285, 41286, 41287, 41288) c6( ) c7(bL, BI, T, BU, BS, BT, BQ, BV, BR, vj, fc, aj, mf, bl, er, J); #c1(ACVRL1) c2(NP_001070869) c3(serine/threonine-protein kinase receptor R3 precursor) c4(34872, 34873) c5(34874, 34875, 34876, 34877, 34878, 34879, 34880, 34881, 34882, 34883, 34884, 34885, 34886, 34887, 34888, 34889) c6( ) c7(ID, T, aC, Cl, e, al, jh, cz, Cg, fr, BW, Cd, Cb, BY, Cj, aE, Cc, pX, kl, Cf, lC, aT, BZ, wC, ly, Ck, xR, aW, vj, hl, Ce, kp, Ca, Ch, Ci, pb, lx, rR, ac, uo, gZ, hR, aS, BX, bC, pB, bt); #c1(ACVRL1) c2(XP_011537310) c3(serine/threonine-protein kinase receptor R3 isoform X2) c4(335009, 335010) c5(335011, 335012, 335013, 335014, 335015, 335016, 335017, 335018, 335019, 335020, 335021, 335022) c6( ) c7(ID, T, aC, Cl, e, al, jh, cz, Cg, fr, BW, Cd, Cb, BY, Cj, aE, Cc, pX, kl, Cf, lC, aT, BZ, wC, ly, Ck, xR, aW, vj, hl, Ce, kp, Ca, Ch, Ci, pb, lx, rR, ac, uo, gZ, hR, aS, BX, bC, pB, bt); #c1(ACY1) c2(NP_001185824) c3(aminoacylase-1 isoform a) c4(76460) c5(76461, 76462, 76463, 76464, 76465) c6( ) c7(mO, ft, bP, eM, Cn, Cm, dq, fr, cm, eC, X, bN); #c1(ACY1) c2(NP_001185825) c3(aminoacylase-1 isoform b) c4(76466) c5(76467, 76468, 76469, 76470, 76471, 76472) c6( ) c7(mO, ft, bP, eM, Cn, Cm, dq, fr, cm, eC, X, bN); #c1(ACY1) c2(NP_001185826) c3(aminoacylase-1 isoform c) c4(76473) c5(76474, 76475, 76476, 76477, 76478, 76479) c6( ) c7(mO, ft, bP, eM, Cn, Cm, dq, fr, cm, eC, X, bN); #c1(ACY1) c2(NP_001185827) c3(aminoacylase-1 isoform d) c4(76480) c5(76481, 76482, 76483, 76484, 76485, 76486) c6( ) c7(mO, ft, bP, eM, Cn, Cm, dq, fr, cm, eC, X, bN); #c1(ACY3) c2(NP_542389) c3(N-acyl-aromatic-L-amino acid amidohydrolase (carboxylate-forming) c4(232323) c5(232324, 232325, 232326, 232327, 232328, 232329) c6( ) c7(cm, aq, T, B, fr); #c1(ACYP2) c2(NP_612457) c3(acylphosphatase-2) c4(236550, 236551) c5(236552, 236553, 236554, 236555, 236556, 236557, 236558, 236559, 236560, 236561, 236562, 236563) c6( ) c7(ff, ht, ac, en); #c1(ADA) c2(NP_000013) c3(adenosine deaminase) c4(54) c5(55, 56, 57, 58, 59, 60) c6( ) c7(bM, T, CR, CH, x, Bc, Co, dV, mH, aa, CG, aW, az, CT, aB, nr, uC, CC, tV, cj, CD, zv, k, Cl, Cx, aj, nA, h, Cr, A, CN, Cs, Cw, Cu, CM, CO, bL, dA, eR, jx, qQ, cz, ti, v, j, J, zn, aT, cy, bq, Cy, ae, CB, iU, oV, Y, r, bN, Cv, ck, CU, iQ, c, CP, Cz, mf, oX, fD, gT, bt, zA, ps, CS, qb, iW, oA, dG, ah, Ct, hF, ad, av, CJ, F, dk, rN, CL, CA, CK, wc, eZ, CF, CE, S, jg, dr, qa, Cq, z, uR, eB, CO, ei, y, xg, X, jr, Cp); #c1(ADAD1) c2(NP_001152757) c3(adenosine deaminase domain-containing protein 1 isoform 2) c4(60143, 60144) c5(60145, 60146, 60147, 60148, 60149, 60150, 60151, 60152, 60153, 60154, 60155, 60156) c6( ) c7(bL, A, cz, hp, j, iV, y, V); #c1(ADAD1) c2(NP_001152767) c3(adenosine deaminase domain-containing protein 1 isoform 3) c4(60173) c5(60174, 60175, 60176, 60177, 60178) c6( ) c7(bL, A, cz, hp, j, iV, y, V); #c1(ADAD1) c2(NP_640336) c3(adenosine deaminase domain-containing protein 1 isoform 1) c4(238436) c5(238437, 238438, 238439, 238440, 238441, 238442, 238443) c6( ) c7(bL, A, cz, hp, j, iV, y, V); #c1(ADAD1) c2(XP_005262802) c3(adenosine deaminase domain-containing protein 1 isoform X3) c4(278094) c5(278095, 278096, 278097, 278098, 278099, 278100) c6( ) c7(bL, A, cz, hp, j, iV, y, V); #c1(ADAM10) c2(NP_001101) c3(disintegrin and metalloproteinase domain-containing protein 10 precursor) c4(41818, 41819) c5(41820, 41821, 41822, 41823, 41824, 41825) c6( ) c7(CV, CX, T, dD, ea, wL, ex, fZ, aA, aj, j, ci, eq, fr, vN, J, ad, eA, cF, db, qK, aT, h, eV, dL, ey, K, H, ae, af, eL, CB, yo, A, d, B, CY, Y, aW, ff, hl, pR, en, t, cm, ek, ck, es, pu, ac, iA, iT, dh, X, U, CZ, dy, m, be, Da, rf, cB, CW, Dc, L); #c1(ADAM10) c2(XP_005254/74) c3(disintegrin and metalloproteinase domain-containing protein 10 isoform X1) c4( ) c5(273043, 273044, 273045, 273046, 273047, 273048) c6( ) c7(CV, CX, T, dD, ea, wL, eX, fZ, aA, aj, j, ci, eq, fr, vN, J, ad, eA, cF, db, qK, aT, h, eV, dL, ey, K, H, ae, af, eL, CB, yo, A, d, B, CY, Y, aW, ff, hl, pR, en, t, cm, ek, ck, es, pu, ac, iA, iT, dh, X, U, CZ, dy, m, be, Da, rF, cB, CW, Dc, L); #c1(ADAM11) c2(NP_002381) c3(disintegrin and metalloproteinase domain-containing protein II preproprotein) c4(127994) c5(127995, 127996, 127997, 127998, 127999, 128000) c6( ) c7(T, iM, ac, je, aW, aw, eX, M, aT, kH, cz, h, eV, i, eA, wD); #c1(ADAM11) c2(XP_005257430) c3 (disintegrin and metalloproteinase domain-containing protein II isoform X1) c4(275106) c5(275107, 275108, 275109, 275110, 275111, 275112) c6( ) c7(T, iM, ac, je, aW, aw, eX, M, aT, kH, cz, h, eV, i, eA, wD); #c1(ADAM11) c2(XP_011523119) c3(disintegrin and metalloproteinase domain-containing protein II isoform X4) c4(317623) c5(317624, 317625, 317626, 317627, 317628, 317629) c6( ) c7(T, iM, ac, je, aW, aw, ex, M, aT, kH, cz, h, eV, i, eA, wD); #c1(ADAM12) c2(NP_001275904) c3(disintegrin and metalloproteinase domain-containing protein 12 isoform 5 preproprotein) c4(107311) c5(107312, 107313, 107314, 107315, 107316, 107317) c6( ) c7(T, Dd, ht, al, aA, eb, v, ci, av, aT, vc, dL, ey, Df, af, cY, d, De, qt, aW, ff, aq, aF, en, g, f, uC, ik, D, kP, eM, m, B); #c1(ADAM12) c2(NP_003465) c3(disintegrin and metalloproteinase domain-containing protein 12 isoform 1 preproprotein) c4(138555) c5(138556, 138557, 138558, 138559, 138560, 138561) c6( ) c7(T, Dd, ht, al, aA, eb, v, ci, av, aT, vc, dl, ey, Df, af, cY, d, De, qt, aW, ff, aq, af, en, g, f, uC, ik, D, KP, eM, m, B); #c1(ADAM12) c2(NP_067673) c3(disintegrin and metalloproteinase domain-containing protein 12 isoform 2 preproprotein) c4(209254) c5(209255, 209256, 209257, 209258, 209259, 209260) c6( ) c7(T, Dd, ht, al, aA, eb, v, ci, av, aT, vc, dl, ey, Df, af, cY, d, De, qt, aW, ff, aq, aF, en, g, f, uC, iK, D, kP, eM, m, B); #c1(ADAM15) c2(NP_001248393) c3(disintegrin and metalloproteinase domain-containing protein 15 isoform 7 preproprotein) c4(93950) c5(93951, 93952, 93953, 93954, 93955, 93956) c6( ) c7(CX, T, kh, dD, Dh, ea, wL, aA, eq, ci, q, ad, aT, Dj, K, ae, Dg, Di, aW, Y, ff, fe, aF, en, g, t, bz, bE, y); #c1(ADAM15) c2(NP_001248394) c3(disintegrin and metalloproteinase domain-containing protein 15 isoform & preproprotein) c4(93957) c5(93958, 93959, 93960, 93961, 93962, 93963) c6( ) c7(CX, T, kh, dD, Dh, ea, wL, aA, eq, ci, q, ad, aT, Dj, K, ae, Dg, Di, aW, Y, ff, fe, aF, en, g, t, bz, bE, y); #c1(ADAM15) c2(NP_003806) c3(disintegrin and metalloproteinase domain-containing protein 15 isoform 2 preproprotein) c4(141022, 141023) c5(141024, 141025, 141026, 141027, 141028, 141029, 141030, 141031, 141032, 141033, 141034, 141035) c6( ) c7(CX, T, kh, dD, Dh, ea, wl, aA, eq, ci, q, ad, aT, Dj, K, ae, Dg, Di, aW, Y, ff, fe, aF, en, g, t, bz, bE, y); #c1(ADAM15) c2(NP_997074) c3(disintegrin and metalloproteinase domain-containing protein 15 isoform 1 preproprotein) c4(265026) c5(265027, 265028, 265029, 265030, 265031, 265032) c6( ) c7(CX, T, kh, dD, Dh, ea, wL, aA, eq, ci, q, ad, aT, Dj, K, ae, Dg, Di, aW, Y, ff, fe, aF, en, g, t, bz, bE, y); #c1(ADAM15) c2(NP_997077) c3(disintegrin and metalloproteinase domain-containing protein 15 isoform 3 preproprotein) c4(265033) c5(265034, 265035, 265036, 265037, 265038, 265039) c6( ) c7(CX, T, kh, dD, Dh, ea, wL, aA, eq, ci, q, ad, aT, Dj, K, ae, Dg, Di, aW, Y, ff, fe, af, en, g, t, bz, bE, y); #c1(ADAM15) c2(NP_997078) c3(disintegrin and metalloproteinase domain-containing protein 15 isoform 4 preproprotein) c4(265040) c5(265041, 265042, 265043, 265044, 265045, 265046) c6( ) c7(CX, T, kh, dD, Dh, ea, wL, aA, eq, ci, q, ad, aT, Dj, K, ae, Dg, Di, aW, Y, ff, fe, af, en, g, t, bz, bE, y); #c1(ADAM15) c2(NP_997079) c3(disintegrin and metalloproteinase domain-containing protein 15 isoform 5 preproprotein) c4(265047) c5(265048, 265049, 265050, 265051, 265052, 265053) c6( ) c7(CX, T, kh, dD, Dh, ea, wL, aA, eq, ci, q, ad, aT, Dj, K, ae, Dg, Di, aW, Y, ff, fe, aF, en, g, t, bz, bE, y); #c1(ADAM15) c2(NP_997080) c3(disintegrin and metalloproteinase domain-containing protein 15 isoform 6 preproprotein) c4(265054) c5(265055, 265056, 265057, 265058, 265059, 265060) c6( ) c7(CX, T, kh, dD, Dh, ea, wl, aA, eq, ci, q, ad, aT, Dj, K, ae, Dg, Di, aW, Y, ff, fe, aF, en, g, t, bz, bE, y); #c1(ADAM17) c2(NP_003174) c3(disintegrin and metalloproteinase domain-containing protein 17 preproprotein) c4(135885, 135886, 135887) c5(135888, 135889, 135890, 135891, 135892, 135893, 135894, 135895, 135896, 135897, 135898, 135899) c6( ) c7(Dk, gh, T, bL, dD, tp, ea, aw, iW, fd, aA, cz, tc, aj, v, j, ci, C, bB, eq, wV, V, fr, q, cd, ad, ai, eA, aE, do, Do, ju, aT, dz, vc, K, Dn, fb, ae, af, hm, DI, B, aW, oV, ff, wc, hl, cr, r, fe, kp, aF, en, eE, t, cm, bz, Dm, pb, ck, es, kP, ac, eM, dB, hk, bE, iA, dh, mx, uz, CZ, dy, m, fD, be, fy, gq, y); #c1(ADAM19) c2(NP_150377) c3(disintegrin and metalloproteinase domain-containing protein 19 preproprotein) c4(228096, 228097) c5(228098, 228099, 228100, 228101, 228102, 228103, 228104, 228105, 228106, 228107, 228108, 228109) c6( ) c7(D, Bv, dB, Dq, jh, fZ, at, Dp, he, dy, m, bK, eA, t, fy, cd, aa); #c1(ADAM19) c2(XP_005266060) c3(disintegrin and metalloproteinase domain-containing protein 19 isoform X1) c4(279899, 279900) c5(279901, 279902, 279903, 279904, 279905, 279906, 279907) c6( ) c7(D, Bv, dB, Dq, jh, fZ, at, Dp, he, dy, m, bK, eA, t, fy, cd, aa); #c1(ADAM19) c2(XP_011532984) c3(disintegrin and metalloproteinase domain-containing protein 19 isoform X2) c4( ) c5(329211, 329212, 329213, 329214, 329215) c6( ) c7(D, Bv, dB, Dq, jh, fZ, at, Dp, he, dy, m, bK, eA, t, fy, cd, aa); #c1(ADAM2) c2(NP_001265042) c3(disintegrin and metalloproteinase domain-containing protein 2 isoform 2 precursor) c4(97713) c5(97714, 97715, 97716, 97717, 97718, 97719) c6( ) c7(es, T, iA, bu); #c1(ADAM2) c2(NP_001455) c3(disintegrin and metalloproteinase domain-containing protein 2 isoform 1 preproprotein) c4(117956) c5(117957, 117958, 117959, 117960, 117961, 117962) c6( ) c7(es, T, iA, bu); #c1(ADAM20) c2(XP_005268209) c3(disintegrin and metalloproteinase domain-containing protein 20 isoform X1) c4( ) c5(281631, 281632, 281633, 281634, 281635) c6( ) c7(aF); #c1(ADAM20) c2(XP_011535574) c3(disintegrin and metalloproteinase domain-containing protein 20 isoform X2) c4(332717) c5(332718) c6( ) c7(aF); #c1(ADAM21) c2(NP_003804) c3(disintegrin and metalloproteinase domain-containing protein 21 preproprotein) c4(14/015) c5(141016, 141017, 141018, 141019, 141020, 141021) c6( ) c7(je, iM, KH); #c1(ADAM22) c2(NP_057435) c3(disintegrin and metalloproteinase domain-containing protein 22 isoform 3 preproprotein) c4(190780) c5(190781, 190782, 190783, 190784, 190785, 190786) c6( ) c7(aW, ha, eA, aT); #c1(ADAM22) c2(NP_068368) c3(disintegrin and metalloproteinase domain-containing protein 22 isoform 2 preproprotein) c4(209332, 209333) c5(209334, 209335, 209336, 209337, 209338, 209339, 209340, 209341, 209342, 209343, 209344, 209345) c6( ) c7(aW, ha, eA, aT); #c1(ADAM22) c2(NP_068369) c3(disintegrin and metalloproteinase domain-containing protein 22 isoform 1 preproprotein) c4(209346) c5(209347, 209348, 209349, 209350, 209351, 209352) c6( ) c7(aW, ha, eA, aT); #c1(ADAM22) c2(XP_011514624) c3(disintegrin and metalloproteinase domain-containing protein 22 isoform X7) c4(307309) c5(307310, 307311, 307312, 307313, 307314, 307315) c6( ) c7(aW, ha, eA, aT); #c1(ADAM23) c2(NP_003803) c3(disintegrin and metalloproteinase domain-containing protein 23 preproprotein) c4(141008) c5(141009, 141010, 141011, 141012, 141013, 141014) c6( ) c7(bM, T, dB, aW, oV, aT, r, aA, fd, aF, q, sj); #c1(ADAM23) c2(XP_005246989) c3 (disintegrin and metalloproteinase domain-containing protein 23 isoform X1) c4(267574, 267575, 267576) c5(267577, 267578, 267579, 267580, 267581, 267582, 267583, 267584, 267585, 267586, 267587, 267588) c6( ) c7(bM, T, dB, aW, oV, aT, r, aA, fd, aF, q, sj); #c1(ADAM23) c2(XP_011510388) c3(disintegrin and metalloproteinase domain-containing protein 23 isoform X2) c4(300919) c5(300920, 300921, 300922, 300923, 300924, 300925, 300926, 300927, 300928, 300929, 300930, 300931, 300932, 300933) c6( ) c7(bM, T, dB, aW, oV, aT, r, aA, fd, aF, q, sj); #c1(ADAM28) c2(NP_055080) c3(disintegrin and metalloproteinase domain-containing protein 28 isoform 1 preproprotein) c4(178846) c5(178847, 178848, 178849, 178850, 178851, 178852) c6( ) c7(bL, T, fd, aA, n, v, ci, q, Dr, aT, aW, oV, ff, r, aF, en, g, bz, eM, dB, cj, ei, B); #c1(ADAM28) c2(NP_068547) c3(disintegrin and metalloproteinase domain-containing protein 28 isoform 3 preproprotein) c4(209393, 209394) c5(209395, 209396, 209397, 209398, 209399, 209400, 209401, 209402, 209403, 209404, 209405, 209406) c6( ) c7(bL, T, fd, aA, n, v, ci, q, Dr, aT, aW, oV, ff, r, aF, en, g, bz, eM, dB, cj, ei, B); #c1(ADAM28) c2(XP_005273437) c3(disintegrin and metalloproteinase domain-containing protein 28 isoform X4) c4(284588) c5(284589, 284590, 284591, 284592, 284593, 284594) c6( ) c7(bL, T, fd, aA, n, v, ci, q, Dr, aT, aW, oV, ff, r, aF, en, g, bz, eM, dB, cj, ei, B); #c1(ADAM28) c2(XP_011542668) c3 (disintegrin and metalloproteinase domain-containing protein 28 isoform X1) c4(341226) c5( ) c6( ) c7(bL, T, fd, aA, n, v, ci, q, Dr, aT, aW, oV, ff, r, aF, en, g, bz, eM, dB, cj, ei, B); #c1(ADAM29) c2(NP_001265056) c3(disintegrin and metalloproteinase domain-containing protein 29 preproprotein) c4(97720, 97721, 97722, 97723) c5(97724, 97725, 97726, 97727, 97728, 97729, 97730, 97731, 97732, 97733, 97734, 97735, 97736, 97737, 97738, 97739, 97740, 97741, 97742, 97743, 97744, 97745, 97746, 97747, 97748, 97749, 97750, 97751, 97752, 97753, 97754, 97755, 97756) c6( ) c7(Y, ff, en, cj); #c1(ADAM33) c2(NP_001269376) c3(disintegrin and metalloproteinase domain-containing protein 33 isoform gamma preproprotein) c4(101353, 101354, 101355, 101356) c5(101357, 101358, 101359, 101360, 101361, 101362) c6( ) c7(bM, Dw, dD, ea, aA, v, j, eq, pM, eA, aT, Ds, A, aW, sp, Du, cr, af, mo, eE, cT, dB, z, at, Dt, k, Dv); #c1(ADAM33) c2(NP_079496) c3(disintegrin and metalloproteinase domain-containing protein 33 isoform alpha preproprotein) c4(218393) c5(218394, 218395, 218396, 218397, 218398, 218399) c6( ) c7(bM, Dw, dD, ea, aA, v, j, eq, pM, eA, aT, Ds, A, aW, sp, Du, cr, af, mo, eE, cT, dB, z, at, Dt, k, Dv); #c1(ADAM33) c2(NP_694882) c3(disintegrin and metalloproteinase domain-containing protein 33 isoform beta preproprotein) c4(245420) c5(245421, 245422, 245423, 245424, 245425, 245426) c6( ) c7(bM, Dw, dD, ea, aA, v, j, eq, pM, eA, aT, Ds, A, aW, sp, Du, cr, aF, mo, eE, cT, dB, z, at, Dt, k, Dv); #c1(ADAM33) c2(XP_006723702) c3(disintegrin and metalloproteinase domain-containing protein 33 isoform X2) c4( ) c5(295298, 295299, 295300, 295301, 295302, 295303) c6( ) c7(bM, Dw, dD, ea, aA, v, j, eq, pM, eA, aT, Ds, A, aW, sp, Du, cr, aF, mo, eE, cT, dB, z, at, Dt, k, Dv); #c1(ADAM33) c2(XP_006723703) c3(disintegrin and metalloproteinase domain-containing protein 33 isoform X4) c4(295304) c5( ) c6( ) c7(bM, Dw, dD, ea, aA, v, j, eq, pM, eA, aT, Ds, A, aW, sp, Du, cr, aF, mo, eE, cT, dB, z, at, Dt, k, Dv); #c1(ADAM33) c2(XP_006723707) c3(disintegrin and metalloproteinase domain-containing protein 33 isoform X9) c4(295305) c5( ) c6( ) c7(bM, Dw, dD, ea, aA, v, j, eq, pM, eA, aT, Ds, A, aW, sp, Du, cr, af, mo, eE, cT, dB, z, at, Dt, k, Dv); #c1(ADAM33) c2(XP_011527669) c3(disintegrin and metalloproteinase domain-containing protein 33 isoform X5) c4(322566) c5(322567, 322568, 322569, 322570, 322571, 322572) c6( ) c7(bM, Dw, dD, ea, aA, v, j, eq, pM, eA, aT, Ds, A, aW, sp, Du, cr, aF, mo, eE, cT, dB, z, at, Dt, k, Dv); #c1(ADAM33) c2(XP_011527673) c3(disintegrin and metalloproteinase domain-containing protein 33 isoform X8) c4(322573, 322574) c5( ) c6( ) c7(bM, Dw, dD, ea, aA, v, j, eq, pM, eA, aT, Ds, A, aW, sp, Du, cr, af, mo, eE, cT, dB, z, at, Dt, k, Dv); #c1(ADAM33) c2(XP_011527674) c3(disintegrin and metalloproteinase domain-containing protein 33 isoform X10) c4( ) c5(322575, 322576, 322577, 322578, 322579, 322580, 322581, 322582, 322583, 322584, 322585, 322586, 322587, 322588, 322589, 322590, 322591, 322592, 322593, 322594, 322595, 322596, 322597, 322598, 322599, 322600) c6( ) c7(bM, Dw, dD, ea, aA, v, j, eq, pM, eA, aT, Ds, A, aW, sp, Du, cr, af, mo, eE, cT, dB, z, at, Dt, k, Dv); #c1(ADAM33) c2(XP_011527675) c3(disintegrin and metalloproteinase domain-containing protein 33 isoform X11) c4( ) c5(322601, 322602, 322603, 322604, 322605, 322606, 322607, 322608, 322609, 322610) c6( ) c7(bM, Dw, dD, ea, aA, v, j, eq, pM, eA, aT, Ds, A, aW, sp, Du, cr, aF, mo, eE, cT, dB, z, at, Dt, k, Dv); #c1(ADAM7) c2(NP_003808) c3(disintegrin and metalloproteinase domain-containing protein 7 preproprotein) c4(141043) c5(141044, 141045, 141046, 141047, 141048, 141049, 141050) c6( ) c7(Y); #c1(ADAM7) c2(XP_005273727) c3(disintegrin and metalloproteinase domain-containing protein 7 isoform X3) c4(284821) c5( ) c6( ) c7(Y); #c1(ADAM7) c2(XP_005273730) c3(disintegrin and metalloproteinase domain-containing protein 7 isoform X6) c4( ) c5(284822, 284823, 284824, 284825, 284826) c6( ) c7(Y); #c1(ADAM8) c2(NP_001100) c3(disintegrin and metalloproteinase domain-containing protein 8 isoform 1 precursor) c4(41811) c5(41812, 41813, 41814, 41815, 41816, 41817) c6( ) c7(bM, T, dD, aC, ea, lH, aw, n, zh, j, eq, eA, ad, ke, av, aE, aT, ae, cW, ff, md, kp, en, g, pl, t, bz, cT, kP, zr, dB, y); #c1(ADAM8) c2(NP_001157961) c3(disintegrin and metalloproteinase domain-containing protein 8 isoform 2 precursor) c4(62959) c5(62960, 62961, 62962, 62963, 62964, 62965) c6( ) c7(bM, T, dD, aC, ea, lH, aw, n, zh, j, eq, eA, ad, ke, av, aE, aT, ae, cW, ff, mQ, kp, en, g, pl, t, bz, cT, kP, zr, dB, y); #c1(ADAM8) c2(NP_001157962) c3(disintegrin and metalloproteinase domain-containing protein 8 isoform 3 precursor) c4(62966) c5(62967, 62968, 62969, 62970, 62971, 62972) c6( ) c7(bM, T, dD, aC, ea, lH, aw, n, zh, j, eq, eA, ad, ke, av, aE, aT, ae, cW, ff, mQ, kp, en, g, pL, t, bz, cT, kP, zr, dB, y); #c1(ADAM8) c2(XP_011537418) c3(disintegrin and metalloproteinase domain-containing protein 8 isoform X1) c4( ) c5(335071) c6( ) c7(bM, T, dD, aC, ea, lH, aw, n, zh, j, eq, eA, ad, ke, av, aE, aT, ae, cW, ff, mQ, kp, en, g, pl, t, bz, cT, kP, zr, dB, y); #c1(ADAM9) c2(NP_003807) c3(disintegrin and metalloproteinase domain-containing protein 9 precursor) c4(141036) c5(141037, 141038, 141039, 141040, 141041, 141042) c6( ) c7(T, dD, id, ea, aA, bB, eq, ci, fr, ad, av, iu, aE, qK, aT, dl, ey, K, Dz, ae, af, eL, lR, aW, Y, ff, Dx, aq, af, en, g, nC, bz, es, Dy, eM, iA, bE, nw, cB, B); #c1(ADAMDEC1) c2(NP_001138743) c3(ADAM DEC1 isoform 2) c4( ) c5(57511, 57512, 57513, 57514, 57515, 57516, 57517, 57518) c6( ) c7(fe, T, vB, ey, uQ); #c1(ADAMDEC1) c2(NP_055294) c3(ADAM DEC1 isoform 1 precursor) c4(180336, 180337) c5(180338, 180339, 180340, 180341, 180342, 180343, 180344, 180345) c6( ) c7(fe, T, vB, ey, uQ); #c1(ADAMTS1) c2(NP_008919) c3(A disintegrin and metalloproteinase with thrombospondin motifs 1 preproprotein) c4(169562) c5(169563, 169564, 169565, 169566, 169567, 169568) c6( ) c7(T, dD, aZ, ea, e, aw, aA, j, ci, eq, V, ad, av, fX, aE, yt, aT, cy, ey, jo, ae, eu, DA, A, aW, eH, vs, aF, cl, eF, f, ac, eM, AM, wO, hL, B, DB); #c1(ADAMTS10) c2(NP_112219) c3(A disintegrin and metalloproteinase with thrombospondin motifs 10 isoform 1 preproprotein) c4(220876, 220877) c5(220878, 220879, 220880, 220881, 220882, 220883, 220884, 220885, 220886, 220887, 220888, 220889) c6( ) c7(dH, DD, eE, DC, DE, K, dw); #c1(ADAMTS10) c2(XP_011526635) c3(A disintegrin and metalloproteinase with thrombospondin motifs 10 isoform X1) c4(320935) c5(320936, 320937, 320938, 320939, 320940, 320941) c6( ) c7(dH, DD, eE, DC, DE, K, dw); #c1(ADAMTS12) c2(NP_112217) c3(A disintegrin and metalloproteinase with thrombospondin motifs 12 preproprotein) c4(220862, 220863) c5(220864, 220865, 220866, 220867, 220868, 220869, 220870, 220871, 220872, 220873, 220874, 220875) c6( ) c7(ai, T, bM, bE, aW, sp, oV, aT, bB, ci, y, bz, cd, DF); #c1(ADAMTS12) c2(XP_011512449) c3(A disintegrin and metalloproteinase with thrombospondin motifs 12 isoform X3) c4(303135) c5( ) c6( ) c7(ai, T, bM, bE, aW, sp, oV, aT, bB, ci, y, bz, cd, DF); #c1(ADAMTS12) c2(XP_011512451) c3(A disintegrin and metalloproteinase with thrombospondin motifs 12 isoform X5) c4( ) c5(303136, 303137, 303138, 303139, 303140, 303141) c6( ) c7(ai, T, bM, bE, aW, sp, oV, aT, bB, ci, y, bz, cd, DF); #c1(ADAMTS13) c2(NP_620594) c3(A disintegrin and metalloproteinase with thrombospondin motifs 13 isoform 1 preproprotein) c4(237864) c5(237865, 237866, 237867, 237868, 237869, 237870, 237871, 237872, 237873, 237874, 237875, 237876, 237877, 237878, 237879, 237880, 237881, 237882, 237883, 237884, 237885, 237886, 237887, 237888, 237889, 237890, 237891, 237892, 237893, 237894, 237895, 237896, 237897, 237898, 237899, 237900, 237901, 237902, 237903, 237904, 237905, 237906, 237907, 237908, 237909, 237910, 237911, 237912, 237913, 237914, 237915, 237916, 237917, 237918, 237919, 237920, 237921, 237922, 237923, 237924, 237925, 237926, 237927, 237928, 237929, 237930, 237931, 237932, 237933, 237934, 237935, 237936, 237937, 237938, 237939, 237940, 237941, 237942, 237943, 237944, 237945, 237946, 237947, 237948, 237949, 237950, 237951, 237952, 237953, 237954, 237955, 237956, 237957, 237958, 237959, 237960, 237961, 237962, 237963, 237964, 237965, 237966, 237967) c6(237968, 237969, 237970, 237971, 237972) c7(P, DS, oO, DM, T, uH, pA, sQ, eR, e, al, DV, jx, aw, n, oY, bS, j, DP, C, v, fr, aY, eh, zo, ai, DJ, DK, ql, M, aT, oW, nm, DO, dV, DU, DG, K, h, DY, DX, jj, qk, vl, eu, s, aW, Y, DN, ev, aq, DR, S, as, iR, cm, uC, DL, ik, DI, dr, ek, ck, ar, DT, ac, xK, iA, dB, ag, DW, AH, X, fP, DO, ei, eg, pJ, kS, B, DH); #c1(ADAMTS13) c2(NP_620595) c3(A disintegrin and metalloproteinase with thrombospondin motifs 13 isoform 3 preproprotein) c4(237973, 237974) c5(237975, 237976, 237977, 237978, 237979, 237980, 237981, 237982, 237983) c6(237984, 237985) c7(P, DS, oO, DM, T, uH, pA, sQ, eR, e, al, DV, jx, aw, n, oY, bS, j, DP, C, v, fr, aY, eh, zo, ai, DJ, DK, ql, M, aT, oW, nm, DO, dV, DU, DG, K, h, DY, DX, jj, qk, vl, eu, s, a, Y, DN, ev, aq, DR, S, as, iR, cm, uC, DL, ik, DI, dr, ek, ck, ar, DT, ac, xK, iA, dB, ag, DW, AH, X, fP, DO, ei, eg, pJ, kS, B, DH); #c1(ADAMTS13) c2(NP_620596) c3(A disintegrin and metalloproteinase with thrombospondin motifs 13 isoform 2 preproprotein) c4(237986) c5(237987, 237988, 237989, 237990, 237991, 237992, 237993, 237994, 237995) c6(237996, 237997) c7(P, DS, oO, DM, T, uH, pA, sQ, eR, e, al, DV, jx, aw, n, oY, bS, j, DP, C, v, fr, aY, eh, zo, ai, DJ, DK, ql, M, aT, oW, nm, DO, dV, DU, DG, K, h, DY, DX, jj, qk, vl, eu, s, aW, Y, DN, ev, aq, DR, S, as, iR, cm, uC, DL, ik, DI, dr, ek, ck, ar, DT, ac, xK, iA, dB, ag, DW, AH, X, fP, DQ, ei, eg, pJ, kS, B, DH); #c1(ADAMTS13) c2(XP_011516477) c3(A disintegrin and metalloproteinase with thrombospondin motifs 13 isoform X2) c4( ) c5(309830, 309831, 309832, 309833, 309834, 309835, 309836, 309837, 309838) c6(309839, 309840) c7(P, DS, oO, DM, T, uH, pA, s, eR, e, al, DV, jx, aw, n, oY, bS, j, DP, C, v, fr, aY, eh, zo, ai, DJ, DK, ql, M, aT, oW, nm, DO, dV, DU, DG, K, h, DY, DX, jj, qk, vl, eu, s, aW, Y, DN, ev, aq, DR, S, as, iR, cm, uC, DL, ik, DI, dr, ek, ck, ar, DT, ac, xK, iA, dB, ag, DW, AH, X, fP, DQ, ei, eg, pJ, kS, B, DH); #c1(ADAMTS13) c2(XP_011516478) c3(A disintegrin and metalloproteinase with thrombospondin motifs 13 isoform X3) c4(309841) c5(309842, 309843, 309844, 309845, 309846, 309847, 309848, 309849, 309850) c6(309851, 309852) c7(P, DS, oO, DM, T, uH, pA, sQ, eR, e, al, DV, jx, aw, n, oY, bS, j, DP, C, v, fr, aY, eh, zo, ai, DJ, DK, ql, M, aT, oW, nm, DO, dV, DU, DG, K, h, DY, DX, jj, qk, vl, eu, s, aW, Y, DN, ev, aq, DR, S, as, iR, cm, uC, DL, ik, DI, dr, ek, ck, ar, DT, ac, xK, iA, dB, ag, DW, AH, X, fP, DQ, ei, eg, pJ, kS, B, DH); #c1(ADAMTS13) c2(XP_011516479) c3(A disintegrin and metalloproteinase with thrombospondin motifs 13 isoform X4) c4( ) c5(309853) c6( ) c7(P, DS, oO, DM, T, uH, pA, s, eR, e, al, DV, jx, aw, n, oY, bS, j, DP, C, v, fr, aY, eh, zo, ai, DJ, DK, ql, M, aT, oW, nm, DO, dV, DU, DG, K, h, DY, DX, jj, qk, vl, eu, s, aW, Y, DN, ev, aq, DR, S, as, iR, cm, uC, DL, ik, DI, dr, ek, ck, ar, DT, ac, xK, iA, dB, ag, DW, AH, X, fP, DQ, ei, eg, pJ, kS, B, DH); #c1(ADAMTS13) c2(XP_011516482) c3(A disintegrin and metalloproteinase with thrombospondin motifs 13 isoform X7) c4(309854, 309855, 309856) c5(309857, 309858, 309859, 309860, 309861, 309862, 309863, 309864, 309865, 309866, 309867, 309868, 309869, 309870, 309871, 309872, 309873, 309874, 309875, 309876, 309877, 309878, 309879, 309880, 309881) c6(309882, 309883, 309884, 309885, 309886, 309887) c7(P, DS, oO, DM, T, uH, pA, sQ, eR, e, al, DV, jx, aw, n, oY, bS, j, DP, C, v, fr, aY, eh, zo, ai, DJ, DK, ql, M, aT, oW, nm, DO, dV, DU, DG, K, h, DY, DX, jj, qk, vl, eu, s, aW, Y, DN, ev, aq, DR, S, as, iR, cm, uC, DL, ik, DI, dr, ek, ck, ar, DT, ac, xK, iA, dB, ag, DW, AH, X, fP, DO, ei, eg, pJ, kS, B, DH); #c1(ADAMTS14) c2(NP_542453) c3(A disintegrin and metalloproteinase with thrombospondin motifs 14 isoform 2 preproprotein) c4(232986) c5(232987, 232988, 232989, 232990, 232991, 232992, 232993, 232994) c6(232995, 232996) c7(V, dt, bK, c, DZ); #c1(ADAMTS14) c2(NP_631894) c3(A disintegrin and metalloproteinase with thrombospondin motifs 14 isoform 1 preproprotein) c4(238191) c5(238192, 238193, 238194, 238195, 238196, 238197, 238198, 238199) c6(238200, 238201) c7(V, dt, bK, c, DZ); #c1(ADAMTS14) c2(XP_011537611) c3(A disintegrin and metalloproteinase with thrombospondin motifs 14 isoform X7) c4( ) c5(335189) c6( ) c7(V, dt, bK, c, DZ); #c1(ADAMTS15) c2(NP_620686) c3(A disintegrin and metalloproteinase with thrombospondin motifs 15 preproprotein) c4(238011) c5(238012, 238013, 238014, 238015, 238016, 238017, 238018, 238019) c6(238020, 238021) c7(bE, aW, oV, r, aT, bB, ci, Ea, q, ad); #c1(ADAMTS16) c2(NP_620687) c3(A disintegrin and metalloproteinase with thrombospondin motifs 16 preproprotein) c4(238022, 238023) c5(238024, 238025, 238026, 238027, 238028, 238029, 238030, 238031, 238032, 238033, 238034, 238035) c6( ) c7(kh, c, ht, cz, hu, an, it); #c1(ADAMTS17) c2(NP_620688) c3(A disintegrin and metalloproteinase with thrombospondin motifs (7 preproprotein) c4(238036) c5(238037, 238038, 238039, 238040, 238041, 238042, 238043) c6( ) c7(Ec, W, DE, aq, Eb); #c1(ADAMTS18) c2(NP_955387) c3(A disintegrin and metalloproteinase with thrombospondin motifs 18 preproprotein) c4(261646, 261647) c5(261648, 261649, 261650, 261651, 261652, 261653, 261654, 261655, 261656, 261657, 261658, 261659) c6( ) c7(T, Y, Be, aT, Ee, ci, j, Ed, fq, bA); #c1(ADAMTS19) c2(NP_598377) c3(A disintegrin and metalloproteinase with thrombospondin motifs 19 preproprotein) c4(235464) c5(235465, 235466, 235467, 235468, 235469, 235470, 235471, 235472) c6(235473, 235474) c7(at, zZ, il, Ef); #c1(ADAMTS2) c2(NP_055059) c3(A disintegrin and metalloproteinase with thrombospondin motifs 2 isoform 1 preproprotein) c4(178653) c5(178654, 178655, 178656, 178657, 178658, 178659, 178660) c6( ) c7(ck, Eg, aW, ds, Ei, aT, cj, fd, gf, bS, K, jj, ct, Z, Eh, DZ); #c1(ADAMTS2) c2(NP_067610) c3(A disintegrin and metalloproteinase with thrombospondin motifs 2 isoform 2 preproprotein) c4(209045) c5(209046, 209047, 209048, 209049, 209050, 209051) c6( ) c7(ck, Eg, aW, ds, Ei, aT, cj, fd, gf, bS, K, jj, ct, Z, Eh, DZ); #c1(ADAMTS20) c2(NP_079279) c3(A disintegrin and metalloproteinase with thrombospondin motifs 20 preproprotein) c4(217666, 217667, 217668, 217669) c5(217670, 217671, 217672, 217673, 217674, 217675, 217676, 217677, 217678, 217679, 217680, 217681, 217682, 217683, 217684, 217685, 217686, 217687, 217688, 217689, 217690, 217691, 217692, 217693) c6( ) c7(Y); #c1(ADAMTS3) c2(NP_055058) c3(A disintegrin and metalloproteinase with thrombospondin motifs 3 preproprotein) c4(178646) c5(178647, 178648, 178649, 178650, 178651, 178652) c6( ) c7(bL, dD, pG, ea, Ej, eo, eq, V, ai, DZ); #c1(ADAMTS4) c2(NP_005090) c3(A disintegrin and metalloproteinase with thrombospondin motifs 4 preproprotein) c4(152805, 152806) c5(152807, 152808, 152809, 152810, 152811, 152812, 152813, 152814, 152815, 152816, 152817, 152818) c6( ) c7(ai, T, dD, d, pG, ea, Eh, Ej, eo, eq, eA, pl, y, V, eN, af, Ek, DZ); #c1(ADAMTS5) c2(NP_008969) c3(A disintegrin and metalloproteinase with thrombospondin motifs 5 preproprotein) c4(169971) c5(169972, 169973, 169974, 169975, 169976, 169977) c6( ) c7(rt, T, dD, d, ea, Ej, eq, eA, y, rA, ai, ad); #c1(ADAMTS6) c2(NP_922932) c3(A disintegrin and metalloproteinase with thrombospondin motifs 6 preproprotein) c4(258654) c5(258655, 258656, 258657, 258658, 258659, 258660, 258661, 258662, 258663, 258664, 258665, 258666, 258667) c6( ) c7(aw); #c1(ADAMTS6) c2(XP_011541418) c3(A disintegrin and metalloproteinase with thrombospondin motifs 6 isoform X3) c4(340023) c5(340024, 340025, 340026, 340027, 340028, 340029) c6( ) c7(aw): #c1(ADAMTS6) c2(XP_011541422) c3(A disintegrin and metalloproteinase with thrombospondin motifs 6 isoform X7) c4(340030) c5( ) c6( ) c7(aw); #c1(ADAMTS7) c2(NP_055087) c3(A disintegrin and metalloproteinase with thrombospondin motifs 7 preproprotein) c4(178867) c5(178868, 178869, 178870, 178871, 178872, 178873, 178874, 178875, 178876, 178877, 178878, 178879) c6( ) c7(dD, kl, W, BL, cW, ea, ds, e, aw, j, eq, y, ai); #c1(ADAMTS7) c2(XP_005254194) c3(A disintegrin and metalloproteinase with thrombospondin motifs 7 isoform X1) c4(273051) c5( ) c6( ) c7(dD, kl, W, BL, cW, ea, ds, e, aw, j, eq, y, ai); #c1(ADAMTS7) c2(XP_011519468) c3(A disintegrin and metalloproteinase with thrombospondin motifs 7 isoform X2) c4( ) c5(314137, 314138) c6( ) c7(dD, kl, W, BL, cW, ea, ds, e, aw, j, eq, y, ai); #c1(ADAMTS8) c2(NP_008968) c3(A disintegrin and metalloproteinase with thrombospondin motifs 8 preproprotein) c4(169957, 169958) c5(169959, 169960, 169961, 169962, 169963, 169964, 169965, 169966, 169967, 169968, 169969, 169970) c6( ) c7(B, aE, dB, aW, aT, ci, K, eA, q); #c1(ADAMTS9) c2(NP_891550) c3(A disintegrin and metalloproteinase with thrombospondin motifs 9 preproprotein) c4(257274) c5(257275, 257276, 257277, 257278, 257279, 257280, 257281, 257282, 257283, 257284, 257285, 257286, 257287, 257288, 257289, 257290) c6( ) c7(bL, T, EI, ht, aw, ja, aA, h, fd, aj, v, aF, ci, hu, hH, it, af, J, ad); #c1(ADAMTS9) c2(XP_005265386) c3(A disintegrin and metalloproteinase with thrombospondin motifs 9 isoform X1) c4(279473, 279474) c5(279475, 279476, 279477, 279478, 279479, 279480, 279481, 279482, 279483) c6( ) c7(bL, T, EI, ht, aw, ja, aA, h, fd, aj, v, af, ci, hu, hH, it, af, J, ad); #c1(ADAMTSL1) c2(NP_001035362) c3(ADAMTS-like protein 1 isoform 4 precursor) c4(32787, 32788, 32789) c5(32790, 32791, 32792, 32793, 32794, 32795, 32796, 32797, 32798, 32799, 32800, 32801, 32802, 32803, 32804, 32805, 32806, 32807, 32808, 32809, 32810, 32811, 32812, 32813, 32814) c6( ) c7(bM, aw, an); #c1(ADAMTSL1) c2(NP_443098) c3(ADAMTS-like protein 1 isoform 2 precursor) c4(229409) c5(229410, 229411, 229412, 229413, 229414, 229415, 229416, 229417, 229418, 229419, 229420, 229421, 229422, 229423, 229424, 229425, 229426, 229427) c6( ) c7(bM, aw, an); #c1(ADAMTSL1) c2(XP_011516366) c3(ADAMTS-like protein 1 isoform X2) c4(309587) c5( ) c6( ) c7(bM, aw, an); #c1(ADAMTSL1) c2(XP_011516368) c3(ADAMTS-like protein 1 isoform X4) c4(309588) c5( ) c6( ) c7(bM, aw, an); #c1(ADAMTSL1) c2(XP_011516372) c3(ADAMTS-like protein 1 isoform X6) c4( ) c5(309589, 309590, 309591, 309592, 309593, 309594, 309595, 309596, 309597) c6( ) c7(bM, aw, an); #c1(ADAMTSL2) c2(NP_001138792) c3(ADAMTS-like protein 2 precursor) c4( ) c5(57755, 57756, 57757, 57758, 57759, 57760, 57761, 57762, 57763) c6( ) c7(mO, xb, DT, up, Em, Eo, DE, Ep, En, rv); #c1(ADAMTSL2) c2(XP_005272294) c3(ADAMTS-like protein 2 isoform X2) c4(283992, 283993) c5(283994, 283995, 283996, 283997, 283998, 283999, 284000, 284001) c6( ) c7(mO, xb, DT, up, Em, Eo, DE, Ep, En, rv); #c1(ADAMTSL2) c2(XP_005272295) c3(ADAMTS-like protein 2 isoform X5) c4(284002) c5( ) c6( ) c7(mO, xb, DT, up, Em, Eo, DE, Ep, En, rv); #c1(ADAMTSL2) c2(XP_011517543) c3(ADAMTS-like protein 2 isoform X3) c4( ) c5(311341, 311342, 311343, 311344, 311345, 311346, 311347) c6( ) c7(mO, xb, DT, up, Em, Eo, DE, Ep, En, rv); #c1(ADAMTSL3) c2(NP_997400) c3(ADAMTS-like protein 3 isoform a precursor) c4(265500) c5(265501, 265502, 265503, 265504, 265505, 265506) c6( ) c7(T, dH, bE, hk, aw, r, bB, ti, bk, q); #c1(ADAMTSL3) c2(XP_011520127) c3(ADAMTS-like protein 3 isoform X5) c4( ) c5(315017, 315018, 315019, 315020) c6( ) c7(T, dH, bE, hk, aw, r, bB, ti, bk, q); #c1(ADAMTSL4) c2(XP_011507946) c3(ADAMTS-like protein 4 isoform X2) c4(298299) c5(298300, 298301) c6( ) c7(x, Et, DT, Eu, Er, Eq, DE, m, k, Ep, t, dr, Es); #c1(ADAMTSL4) c2(XP_011507947) c3(ADAMTS-like protein 4 isoform X3) c4(298302) c5(298303, 298304, 298305) c6( ) c7(x, Et, DT, Eu, Er, Eq, DE, m, k, Ep, t, dr, Es); #c1(ADAMTSL4) c2(XP_011507952) c3(ADAMTS-like protein 4 isoform X5) c4( ) c5(298306) c6( ) c7(x, Et, DT, Eu, Er, Eq, DE, m, k, Ep, t, dr, Es); #c1(ADAMTSL4) c2(XP_011507954) c3(ADAMTS-like protein 4 isoform X6) c4(298307) c5(298308, 298309, 298310, 298311, 298312, 298313, 298314) c6( ) c7(x, Et, DT, Eu, Er, Eq, DE, m, k, Ep, t, dr, Es); #c1(ADAMTSL5) c2(NP_998769) c3(ADAMTS-like protein 5 precursor) c4(265882) c5(265883, 265884, 265885, 265886, 265887, 265888, 265889, 265890, 265891) c6(265892, 265893) c7(ac); #c1(ADAMTSL5) c2(XP_005259606) c3(ADAMTS-like protein 5 isoform X2) c4(276189) c5(276190, 276191, 276192, 276193, 276194, 276195, 276196, 276197) c6(276198, 276199) c7(ac); #c1(ADAMTSL5) c2(XP_011526264) c3(ADAMTS-like protein 5 isoform X3) c4(320536) c5( ) c6( ) c7(ac); #c1(ADAMTSL5) c2(XP_011526265) c3(ADAMTS-like protein 5 isoform X4) c4( ) c5(320537, 320538, 320539, 320540, 320541, 320542, 320543) c6(320544, 320545) c7(ac); #c1(ADAP1) c2(NP_001271237) c3(arf-GAP with dual PH domain-containing protein 1 isoform 2) c4(103693) c5(103694, 103695, 103696, 103697, 103698, 103699) c6( ) c7(cm, af, L); #c1(ADAP1) c2(NP_001271239) c3(arf-GAP with dual PH domain-containing protein 1 isoform 3) c4( ) c5(103700, 103701, 103702, 103703, 103704, 103705) c6( ) c7(cm, af, L); #c1(ADAP1) c2(NP_001271240) c3(arf-GAP with dual PH domain-containing protein 1 isoform 4) c4(103706) c5(103707, 103708, 103709, 103710, 103711, 103712, 103713, 103714, 103715, 103716, 103717, 103718) c6( ) c7(cm, af, L); #c1(ADAP1) c2(NP_006860) c3(arf-GAP with dual PH domain-containing protein 1 isoform 1) c4(168686, 168687, 168688) c5(168689, 168690, 168691, 168692, 168693, 168694) c6( ) c7(cm, af, L); #c1(ADAP2) c2(NP_060874) c3(arf-GAP with dual PH domain-containing protein 2) c4(198223) c5(198224, 198225, 198226, 198227, 198228, 198229) c6( ) c7(T, Ew, nX, Az, Ev, hP, bD, xb, xt); #c1(ADAR) c2(NP_001020278) c3(double-stranded RNA-specific adenosine deaminase isoform d) c4(28529) c5(28530, 28531, 28532, 28533, 28534) c6( ) c7(bM, T, Ez, lk, fR, al, jx, pl, jE, EA, hX, EB, F, ds, aU, h, lQ, cp, kw, Y, eZ, r, lo, CQ, as, Ey, f, ps, EC, c, dB, AH, Da, rF, it, B, Ex); #c1(ADAR) c2(NP_001102) c3(double-stranded RNA-specific adenosine deaminase isoform a) c4(41826) c5(41827, 41828, 41829, 41830, 41831, 41832) c6( ) c7(bM, T, Ez, lk, fR, al, jx, pl, jE, EA, hX, EB, F, ds, aU, h, lQ, cp, kw, Y, eZ, r, lo, CQ, as, Ey, f, ps, EC, c, dB, AH, Da, rF, it, B, Ex); #c1(ADAR) c2(NP_056655) c3(double-stranded RNA-specific adenosine deaminase isoform b) c4(188112) c5( ) c6( ) c7(bM, T, Ez, lk, fR, al, jx, pl, jE, EA, hX, EB, F, ds, aU, h, lQ, cp, kw, Y, eZ, r, lo, CO, as, Ey, f, ps, EC, c, dB, AH, Da, rF, it, B, Ex); #c1(ADAR) c2(XP_011507362) c3(double-stranded RNA-specific adenosine deaminase isoform X2) c4(297193) c5(297194, 297195, 297196, 297197, 297198, 297199, 297200) c6( ) c7(bM, T, Ez, lk, fR, al, jx, pl, jE, EA, hX, EB, F, ds, aU, h, lQ, cp, kw, Y, eZ, r, lo, CO, as, Ey, f, ps, EC, c, dB, AH, Da, rF, it, B, Ex); #c1(ADAR) c2(XP_011507363) c3(double-stranded RNA-specific adenosine deaminase isoform X3) c4( ) c5(297201, 297202, 297203, 297204, 297205, 297206) c6( ) c7(bM, T, Ez, lk, fR, al, jx, pl, jE, EA, hX, EB, F, ds, aU, h, lQ, cp, kw, Y, eZ, r, lo, CO, as, Ey, f, ps, EC, c, dB, AH, Da, rF, it, B, Ex); #c1(ADARB1) c2(NP_056649) c3(double-stranded RNA-specific editase 1 isoform 3) c4(188099) c5(188100, 188101, 188102, 188103, 188104) c6( ) c7(T, al, ex, bO, n, eV, bK, oi, he, hH, kl, d, eZ, aq, as, bz, c, cA, dB, eM, B, EB); #c1(ADARB1) c2(XP_006724017) c3(double-stranded RNA-specific editase 1 isoform X4) c4( ) c5(295599, 295600, 295601, 295602, 295603, 295604, 295605) c6( ) c7(T, al, eX, bO, n, eV, bK, oi, he, hH, kl, d, eZ, aq, as, bz, c, cA, dB, eM, B, EB); #c1(ADARB1) c2(XP_006724019) c3(double-stranded RNA-specific editase 1 isoform X2) c4(295606) c5(295607, 295608, 295609, 295610, 295611, 295612, 295613, 295614, 295615, 295616, 295617, 295618, 295619, 295620, 295621, 295622) c6( ) c7(T, al, ex, b, n, eV, bK, oi, he, hH, kl, d, eZ, aq, as, bz, c, cA, dB, eM, B, EB); #c1(ADARB1) c2(XP_011527727) c3(double-stranded RNA-specific editase 1 isoform X3) c4(322685) c5(322686, 322687, 322688, 322689) c6( ) c7(T, al, eX, bO, n, eV, bK, oi, he, hH, kl, d, eZ, aq, as, bz, c, cA, dB, eM, B, EB); #c1(ADARB1) c2(XP_011527729) c3 (double-stranded RNA-specific editase 1 isoform X1) c4(322690, 322691) c5(322692, 322693, 322694, 322695, 322696, 322697) c6( ) c7(T, al, ex, bO, n, eV, bK, oi, he, hH, kl, d, eZ, aq, as, bz, c, cA, dB, eM, B, EB); #c1(ADARB1) c2(XP_011527733) c3(double-stranded RNA-specific editase 1 isoform X7) c4( ) c5(322698, 322699, 322700, 322701, 322702) c6( ) c7(T, al, eX, bO, n, eV, bK, oi, he, hH, kl, d, eZ, aq, as, bz, c, cA, dB, eM, B, EB); #c1(ADARB2) c2(NP_061172) c3(double-stranded RNA-specific editase B2) c4(199733, 199734, 199735) c5(199736, 199737, 199738, 199739, 199740, 199741, 199742, 199743, 199744, 199745, 199746, 199747, 199748, 199749, 199750, 199751, 199752, 199753) c6( ) c7(T, d, ac, c, dB, fq, Ea, lB, sD); #c1(ADAT2) c2(NP_001273188) c3(tRNA-specific adenosine deaminase 2 isoform b) c4( ) c5(104526, 104527, 104528, 104529, 104530) c6( ) c7(nM); #c1(ADAT2) c2(NP_872309) c3(tRNA-specific adenosine deaminase 2 isoform a) c4(256079, 256080) c5(256081, 256082, 256083, 256084, 256085, 256086) c6( ) c7(nM); #c1(ADAT2) c2(XP_011533741) c3(tRNA-specific adenosine deaminase 2 isoform X2) c4( ) c5(329846, 329847) c6( ) c7(nM); #c1(ADAT2) c2(XP_011533745) c3(tRNA-specific adenosine deaminase 2 isoform X3) c4(329848) c5(329849, 329850, 329851, 329852, 329853) c6( ) c7(nM); #c1(ADAT3) c2(NP_612431) c3(probable inactive tRNA-specific adenosine deaminase-like protein 3) c4(236453, 236454) c5(236455, 236456, 236457, 236458, 236459, 236460, 236461, 236462, 236463, 236464, 236465, 236466, 236467) c6( ) c7(ED); #c1(ADCK3) c2(NP_064632) c3(chaperone activity of bel complex-like, mitochondrial) c4(203405, 203406, 203407, 203408, 203409, 203410, 203411) c5(203412, 203413, 203414, 203415, 203416, 203417, 203418, 203419, 203420, 203421, 203422, 203423, 203424, 203425, 203426, 203427, 203428, 203429, 203430, 203431, 203432, 203433, 203434, 203435, 203436, 203437, 203438, 203439, 203440, 203441, 203442, 203443, 203444, 203445, 203446, 203447, 203448, 203449, 203450, 203451, 203452, 203453, 203454, 203455, 203456, 203457) c6( ) c7(EG, EF, EH, ky, EE); #c1(ADCK4) c2(NP_001136027) c3(aarf domain-containing protein kinase 4 isoform b) c4(54058) c5(54059, 54060, 54061, 54062, 54063, 54064, 54065, 54066, 54067, 54068) c6( ) c7(dy, ah, gi, EI, lf); #c1(ADCK4) c2(XP_005259327) c3(aarF domain-containing protein kinase 4 isoform X2) c4( ) c5(276061) c6( ) c7(dy, ah, gi, EI, lf); #c1(ADCK4) c2(XP_006723456) c3(aarF domain-containing protein kinase 4 isoform X1) c4(295208) c5(295209, 295210, 295211, 295212, 295213) c6( ) c7(dy, ah, gi, EI, lf); #c1(ADCY1) c2(NP_066939) c3(adenylate cyclase type 1 isoform 1) c4(207601, 207602) c5(207603, 207604, 207605, 207606, 207607, 207608, 207609, 207610, 207611, 207612, 207613, 207614, 207615) c6( ) c7(ft, cA, hr, hR, bS, ct, Z); #c1(ADCY10) c2(NP_001161221) c3(adenylate cyclase type 10 isoform 2) c4( ) c5(65514, 65515, 65516, 65517, 65518) c6( ) c7(T, s, DD, zs, EK, cr, r, cz, Ej, ci, lQ, EL, eA, q, bA, EJ); #c1(ADCY10) c2(NP_001284701) c3(adenylate cyclase type 10 isoform 3) c4(112342, 112343) c5(112344, 112345, 112346, 112347, 112348, 112349) c6( ) c7(T, s, DD, zs, EK, cr, r, cz, Ej, ci, lQ, EL, eA, q, bA, EJ); #c1(ADCY10) c2(NP_060887) c3(adenylate cyclase type 10 isoform 1) c4(198396) c5(198397, 198398, 198399, 198400, 198401, 198402, 198403, 198404, 198405, 198406, 198407, 198408) c6( ) c7(T, s, DD, zs, EK, cr, r, cz, Ej, ci, lQ, EL, eA, q, bA, EJ); #c1(ADCY10) c2(XP_006711512) c3(adenylate cyclase type 10 isoform X6) c4(286046) c5( ) c6( ) c7(T, s, DD, zs, EK, cr, r, cz, Ej, ci, lQ, EL, eA, q, bA, EJ); #c1(ADCY10) c2(XP_011508066) c3(adenylate cyclase type 10 isoform X7) c4( ) c5(298533) c6( ) c7(T, s, DD, zs, EK, cr, r, cz, Ej, ci, lQ, EL, eA, q, bA, EJ); #c1(ADCY2) c2(NP_065433) c3(adenylate cyclase type 2) c4(204759, 204760, 204761, 204762) c5(204763, 204764, 204765, 204766, 204767, 204768, 204769, 204770, 204771, 204772, 204773, 204774, 204775, 204776, 204777, 204778, 204779, 204780, 204781, 204782, 204783) c6( ) c7(bM, T, EM, ac, bE, bB, at, pM, cm, eN, eA); #c1(ADCY2) c2(XP_011512244) c3(adenylate cyclase type 2 isoform X1) c4( ) c5(302818, 302819, 302820, 302821, 302822) c6( ) c7(bM, T, EM, ac, bE, bB, at, pM, cm, eN, eA); #c1(ADCY3) c2(NP_004027) c3(adenylate cyclase type 3) c4(142942, 142943) c5(142944, 142945, 142946, 142947, 142948, 142949, 142950, 142951, 142952, 142953, 142954, 142955) c6( ) c7(EN, bL, T, bO, aA, dq, v, bK, aF, fq); #c1(ADCY3) c2(XP_005264161) c3 (adenylate cyclase type 3 isoform X7) c4(278677) c5( ) c6( ) c7(EN, bL, T, bO, aA, dq, v, bK, aF, fq); #c1(ADCY3) c2(XP_011530796) c3(adenylate cyclase type 3 isoform X9) c4( ) c5(326850, 326851, 326852, 326853, 326854, 326855) c6( ) c7(EN, bL, T, bO, aA, dq, v, bK, aF, fq); #c1(ADCY5) c2(NP_001186571) c3(adenylate cyclase type 5 isoform 2) c4(77869) c5(77870, 77871, 77872, 77873, 77874, 77875) c6( ) c7(bL, cc, zQ, mg, cz, EO, aU, bO, bS, v, ct, K, ci, Z, zP, xw, bA); #c1(ADCY5) c2(NP_899200) c3(adenylate cyclase type 5 isoform 1) c4(258012) c5(258013, 258014, 258015, 258016, 258017, 258018) c6( ) c7(bL, cc, zQ, mg, cz, EO, aU, bO, bS, v, ct, K, ci, Z, zP, xw, bA); #c1(ADCY5) c2(XP_006713546) c3(adenylate cyclase type 5 isoform X6) c4(287223) c5(287224, 287225, 287226, 287227, 287228, 287229) c6( ) c7(bL, cc, zQ, mg, cz, ED, aU, bO, bS, v, ct, K, ci, Z, zP, xw, bA); #c1(ADCY6) c2(NP_056085) c3(adenylate cyclase type 6 isoform a) c4(185603, 185604) c5(185605, 185606, 185607, 185608, 185609, 185610, 185611, 185612, 185613, 185614, 185615, 185616, 185617, 185618, 185619, 185620, 185621) c6( ) c7(qb, EP, cd); #c1(ADCY6) c2(XP_011536115) c3 (adenylate cyclase type 6 isoform X4) c4( ) c5(333433) c6( ) c7(qb, EP, cd); #c1(ADCY7) c2(NP_001105) c3(adenylate cyclase type 7 isoform 1) c4(42311) c5(42312, 42313, 42314, 42315, 42316, 42317, 42318, 42319, 42320, 42321) c6( ) c7(cA, bO, bS, ct, Z, he, bt); #c1(ADCY7) c2(XP_005255843) c3(adenylate cyclase type 7 isoform X1) c4(273886, 273887) c5(273888, 273889, 273890, 273891, 273892, 273893) c6( ) c7(cA, bO, bS, ct, Z, he, bt); #c1(ADCY7) c2(XP_011521146) c3 (adenylate cyclase type 7 isoform X5) c4( ) c5(316102, 316103, 316104, 316105, 316106, 316107) c6( ) c7(cA, bO, bS, ct, Z, he, bt); #c1(ADCY8) c2(NP_001106) c3(adenylate cyclase type 8) c4(42787) c5( ) c6( ) c7(kT, dD, cF, cA, ea, aw, EQ, bS, bK, ct, eq, Z, af); #c1(ADCY9) c2(NP_001107) c3(adenylate cyclase type 9) c4( ) c5( ) c6( ) c7(bM, gj, ac, qe, cA, nX, eh, dV, cz, fF, lr, bK, ER, eg, fq, cd); #c1(ADCYAP1) c2(NP_001093203) c3(pituitary adenylate cyclase-activating polypeptide preproprotein) c4( ) c5(39919, 39920, 39921, 39922, 39923, 39924, 39925, 39926, 39927, 39928, 39929, 39930, 39931, 39932) c6( ) c7(bL, gh, T, aw, fd, aj, uu, aY, V, eA, J, ad, av, mj, cc, dY, aE, lG, cy, bk, K, bq, he, ae, hM, EX, EV, ES, EU, KD, EZ, cm, bz, hq, ET, EW, ac, c, bP, dB, Ei, U, dX, EY, ei, it, X); #c1(ADCYAP1) c2(XP_005258138) c3(pituitary adenylate cyclase-activating polypeptide isoform X1) c4(275518, 275519) c5(275520, 275521, 275522, 275523, 275524, 275525) c6( ) c7(bL, gh, T, aw, fd, aj, uu, aY, V, eA, J, ad, av, mj, cc, dY, aE, lG, cy, bK, K, bq, he, ae, hM, EX, EV, ES, EU, KD, EZ, cm, bz, hq, ET, EW, ac, c, bP, dB, Ei, U, dX, EY, ei, it, X); #c1(ADCYAP1RI) c2(NP_001109) c3(pituitary adenylate cyclase-activating polypeptide type 1 receptor isoform 3 precursor) c4(43278, 43279) c5(43280, 43281, 43282, 43283, 43284, 43285) c6( ) c7(bM, ul, EQ, fd, bS, uu, ct, Fb, pM, hF, Fc, eA, ad, av, pX, lG, bK, K, he, as, Fa, kp, Z, cm, bz, hq, W, bP, Ei, m, EY, pk, X); #c1(ADCYAP1RI) c2(NP_001186564) c3(pituitary adenylate cyclase-activating polypeptide type 1 receptor isoform 1 precursor) c4(77835) c5(77836, 77837, 77838, 77839, 77840, 77841) c6( ) c7(bM, ul, EQ, fd, bS, uu, ct, Fb, pM, hF, Fc, eA, ad, av, pX, lG, bK, K, he, ae, Fa, kp, Z, cm, bz, hq, W, bP, Ei, m, EY, pk, X); #c1(ADCYAP1RI) c2(NP_001186566) c3 (pituitary adenylate cyclase-activating polypeptide type 1 receptor isoform 4 precursor) c4(77842) c5(77843, 77844, 77845, 77846, 77847, 77848, 77849, 77850, 77851, 77852, 77853, 77854) c6( ) c7(bM, ul, EQ, fd, bS, uu, ct, Fb, pM, hF, Fc, eA, ad, av, pX, lG, bK, K, he, ae, Fa, kp, Z, cm, bz, hq, W, bP, Ei, m, EY, pK, X); #c1(ADCYAP1RI) c2(XP_006715708) c3(pituitary adenylate cyclase-activating polypeptide type 1 receptor isoform X4) c4(289733) c5(289734, 289735, 289736, 289737, 289738, 289739) c6( ) c7(bM, ul, EQ, fd, bS, uu, ct, Fb, pM, hF, Fc, eA, ad, av, pX, lG, bK, K, he, ae, Fa, kp, Z, cm, bz, hq, W, bP, Ei, m, EY, pk, X); #c1(ADCYAP1RI) c2(XP_011513419) c3(pituitary adenylate cyclase-activating polypeptide type 1 receptor isoform X1) c4(305659) c5(305660, 305661, 305662, 305663, 305664, 305665, 305666) c6( ) c7(bM, ul, EQ, fd, bS, uu, ct, Fb, pM, hF, Fc, eA, ad, av, pX, lG, bK, K, he, ae, Fa, kp, Z, cm, bz, hq, W, bP, Ei, m, EY, pK, X); #c1(ADCYAP1RI) c2(XP_011513422) c3(pituitary adenylate cyclase-activating polypeptide type 1 receptor isoform X5) c4(305667) c5(305668, 305669, 305670, 305671, 305672) c6( ) c7(bM, ul, EQ, fd, bS, uu, ct, Fb, pM, hF, Fc, eA, ad, av, pX, lG, bK, K, he, ae, Fa, kp, Z, cm, bz, hq, W, bP, Ei, m, EY, pK, X); #c1(ADD1) c2(NP_001110) c3(alpha-adducin isoform a) c4(43286) c5(43287, 43288, 43289, 43290, 43291, 43292, 43293, 43294, 43295, 43296) c6( ) c7(bL, dD, Fj, eR, ea, De, e, uv, aw, cz, eX, oR, aj, v, j, eq, il, aY, cd, J, dY, ec, M, eV, ey, Fg, eu, sj, FI, A, Fe, Ff, pD, cM, uC, qa, D, kT, rR, ac, Fd, c, Fh, Fi, wB, Fk, aS, dy, bt, B); #c1(ADD1) c2(NP_001273574) c3(alpha-adducin isoform e) c4(105324) c5(105325, 105326, 105327, 105328, 105329, 105330) c6( ) c7(bL, dD, Fj, eR, ea, oe, e, uv, aw, cz, ex, OR, aj, v, j, eq, il, aY, cd, J, dY, ec, M, eV, ey, Fg, eu, sj, FI, A, Fe, Ff, pD, cM, uC, qa, D, KT, rR, ac, Fd, c, Fh, Fi, wB, Fk, aS, dy, bt, B); #c1(ADD1) c2(NP_054908) c3(alpha-adducin isoform b) c4(178279, 178280) c5(178281, 178282, 178283, 178284, 178285, 178286, 178287, 178288, 178289, 178290, 178291, 178292) c6( ) c7(bL, dD, Fj, eR, ea, oe, e, uv, aw, cz, ex, oR, aj, v, j, eq, il, aY, cd, J, dY, ec, M, eV, ey, Fg, eu, sj, Fl, A, Fe, Ff, pD, cM, uC, qa, D, kT, rR, ac, Fd, c, Fh, Fi, wB, Fk, aS, dy, bt, B); #c1(ADD1) c2(NP_054909) c3(alpha-adducin isoform c) c4(178293) c5(178294, 178295, 178296, 178297, 178298, 178299, 178300, 178301, 178302, 178303, 178304, 178305, 178306, 178307, 178308, 178309, 178310, 178311) c6( ) c7(bL, dD, Fj, eR, ea, oe, e, uv, aw, cz, eX, oR, aj, v, j, eq, il, aY, cd, J, dY, ec, M, eV, ey, Fg, eu, sj, FI, A, Fe, Ff, pD, cM, uC, qa, D, KT, rR, ac, Fd, c, Fh, Fi, wB, Fk, aS, dy, bt, B); #c1(ADD1) c2(NP_789771) c3(alpha-adducin isoform d) c4(252232) c5(252233, 252234, 252235, 252236, 252237, 252238, 252239, 252240, 252241, 252242) c6( ) c7(bL, dD, Fj, eR, ea, oe, e, uv, aw, cz, eX, oR, aj, v, j, eq, il, aY, cd, J, dY, ec, M, eV, ey, Fg, eu, sj, FI, A, Fe, Ff, pD, cM, uC, qa, D, kT, rR, ac, Fd, c, Fh, Fi, wB, Fk, aS, dy, bt, B); #c1(ADD1) c2(XP_005247992) c3(alpha-adducin isoform X2) c4(268172) c5( ) c6( ) c7(bL, dD, Fj, eR, ea, oe, e, uv, aw, cz, ex, oR, aj, v, j, eq, il, aY, cd, J, dY, ec, M, eV, ey, Fg, eu, sj, FI, A, Fe, Ff, pD, cM, uC, qa, D, KT, rR, ac, Fd, c, Fh, Fi, wB, Fk, aS, dy, bt, B); #c1(ADD1) c2(XP_005247993) c3(alpha-adducin isoform X3) c4(268173) c5( ) c6( ) c7(bL, dD, Fj, eR, ea, oe, e, uv, aw, cz, ex, OR, aj, v, j, eq, il, aY, cd, J, dY, ec, M, eV, ey, Fg, eu, sj, FI, A, Fe, Ff, pD, cM, uC, qa, D, kT, rR, ac, Fd, c, Fh, Fi, wB, Fk, aS, dy, bt, B); #c1(ADD2) c2(NP_001171984) c3(beta-adducin isoform f) c4(72110) c5(72111, 72112, 72113, 72114, 72115, 72116, 72117, 72118, 72119, 72120, 72121, 72122) c6( ) c7(Fl, Fp, A, Fn, Fd, Fh, Fm, oe, Fo, yL, cz, ey, S, bt, qa); #c1(ADD2) c2(NP_001608) c3(beta-adducin isoform a) c4(119128, 119129, 119130, 119131, 119132, 119133) c5(119134, 119135, 119136, 119137, 119138, 119139, 119140, 119141, 119142, 119143, 119144, 119145, 119146, 119147, 119148, 119149, 119150, 119151, 119152) c6( ) c7(FI, Fp, A, Fn, Fd, Fh, Fm, oe, Fo, yL, cz, ey, S, bt, qa); #c1(ADD2) c2(NP_059516) c3(beta-adducin isoform b) c4(193338) c5(193339, 193340, 193341, 193342, 193343, 193344, 193345, 193346, 193347, 193348, 193349, 193350, 193351, 193352, 193353, 193354, 193355, 193356, 193357, 193358, 193359, 193360, 193361, 193362, 193363) c6( ) c7(FI, Fp, A, Fn, Fd, Fh, Fm, oe, Fo, yL, cz, ey, S, bt, qa); #c1(ADD2) c2(NP_059522) c3(beta-adducin isoform e) c4(193364, 193365) c5(193366, 193367, 193368, 193369, 193370, 193371, 193372, 193373, 193374, 193375, 193376, 193377) c6( ) c7(FI, Fp, A, Fn, Fd, Fh, Fm, oe, Fo, yL, cz, ey, S, bt, qa); #c1(ADD3) c2(NP_058432) c3(gamma-adducin isoform a) c4(192702) c5(192703, 192704, 192705, 192706, 192707, 192708) c6( ) c7(Fp, d, W, Fr, Fh, Ft, U, cz, oR, Fq, eu, iq, Fs); #c1(ADGB) c2(NP_078970) c3 (androglobin) c4(216347) c5(216348, 216349, 216350, 216351, 216352, 216353, 216354, 216355, 216356, 216357, 216358, 216359) c6( ) c7(AW); #c1(ADGB) c2(XP_011534433) c3 (androglobin isoform X7) c4(330935) c5( ) c6( ) c7(AW); #c1(ADGB) c2(XP_011534436) c3(androglobin isoform X10) c4(330936) c5( ) c6( ) c7(AW); #c1(ADGB) c2(XP_011534437) c3(androglobin isoform X11) c4( ) c5(330937, 330938, 330939, 330940, 330941, 330942) c6( ) c7(AW); #c1(ADGRA1) c2(NP_001077378) c3(probable G-protein coupled receptor (23 isoform 1) c4(37788, 37789) c5(37790, 37791, 37792, 37793, 37794, 37795, 37796, 37797, 37798, 37799, 37800, 37801) c6( ) c7(bM, mQ); #c1(ADGRA1) c2(XP_005252752) c3(probable G-protein coupled receptor (23 isoform X1) c4(272039, 272040) c5(272041, 272042, 272043, 272044, 272045, 272046, 272047, 272048, 272049, 272050, 272051, 272052) c6( ) c7(bM, mQ); #c1(ADGRA2) c2(NP_116166) c3(G-protein coupled receptor 124 precursor) c4(225501, 225502, 225503) c5(225504, 225505, 225506, 225507, 225508, 225509, 225510, 225511, 225512, 225513, 225514, 225515) c6( ) c7(gv, cY); #c1(ADGRA2) c2(XP_005273528) c3(G-protein coupled receptor (24 isoform X4) c4( ) c5(284669, 284670, 284671, 284672, 284673, 284674) c6( ) c7(gv, cY); #c1(ADGRA2) c2(XP_011542783) c3(G-protein coupled receptor 124 isoform X1) c4( ) c5(341391) c6( ) c7(gv, cY); #c1(ADGRA3) c2(NP_660333) c3(probable G-protein coupled receptor (25 precursor) c4(240972, 240973) c5(240974, 240975, 240976, 240977, 240978, 240979, 240980, 240981, 240982, 240983, 240984, 240985) c6( ) c7(aj, cF, Fu); #c1(ADGRA3) c2(XP_011512113) c3 (probable G-protein coupled receptor 125 isoform X2) c4(302726) c5(302727, 302728, 302729, 302730, 302731, 302732) c6( ) c7(aj, cF, Fu); #c1(ADGRB1) c2(XP_011515501) c3(brain-specific angiogenesis inhibitor 1 isoform X1) c4(308342) c5(308343, 308344, 308345, 308346, 308347, 308348, 308349, 308350, 308351, 308352) c6( ) c7(T, d, dB, r, aA, cy, aF, aS, K, q, eA, B); #c1(ADGRB1) c2(XP_011515503) c3(brain-specific angiogenesis inhibitor 1 isoform X3) c4( ) c5(308353) c6( ) c7(T, d, dB, r, aA, cy, aF, aS, K, q, eA, B); #c1(ADGRB1) c2(XP_011515504) c3(brain-specific angiogenesis inhibitor 1 isoform X4) c4( ) c5(308354, 308355, 308356, 308357) c6( ) c7(T, d, dB, r, aA, cy, af, aS, K, q, eA, B); #c1(ADGRB2) c2(NP_001281264) c3(brain-specific angiogenesis inhibitor 2 isoform 1 preproprotein) c4(111614) c5(1/1615, 111616, 111617, 111618, 111619, 111620) c6( ) c7(dB, cy, cF, ek); #c1(ADGRB2) c2(NP_001281265) c3(brain-specific angiogenesis inhibitor 2 isoform 2 preproprotein) c4(111621) c5(111622, 111623, 111624, 111625, 111626, 111627, 111628, 111629, 111630, 111631) c6( ) c7(dB, cy, cF, ek); #c1(ADGRB2) c2(XP_011540150) c3(brain-specific angiogenesis inhibitor 2 isoform X1) c4(338505) c5(338506, 338507, 338508, 338509, 338510, 338511) c6( ) c7(dB, cy, cF, ek); #c1(ADGRB2) c2(XP_011540152) c3(brain-specific angiogenesis inhibitor 2 isoform X3) c4(338512) c5(338513, 338514, 338515, 338516, 338517, 338518) c6( ) c7(dB, cy, cF, ek); #c1(ADGRB2) c2(XP_011540154) c3(brain-specific angiogenesis inhibitor 2 isoform X5) c4(338519, 338520) c5(338521, 338522, 338523, 338524, 338525, 338526, 338527, 338528, 338529, 338530, 338531, 338532) c6( ) c7(dB, cy, cF, ek); #c1(ADGRB2) c2(XP_011540156) c3(brain-specific angiogenesis inhibitor 2 isoform X7) c4(338533) c5(338534, 338535, 338536, 338537, 338538, 338539) c6( ) c7(dB, cy, cF, ek); #c1(ADGRB2) c2(XP_011540157) c3(brain-specific angiogenesis inhibitor 2 isoform X8) c4(338540) c5(338541, 338542, 338543, 338544, 338545, 338546) c6( ) c7(dB, cy, cF, ek); #c1(ADGRB2) c2(XP_011540158) c3(brain-specific angiogenesis inhibitor 2 isoform X9) c4(338547) c5(338548, 338549, 338550, 338551, 338552, 338553) c6( ) c7(dB, cy, cF, ek); #c1(ADGRB3) c2(NP_001695) c3(brain-specific angiogenesis inhibitor 3) c4( ) c5(120101, 120102, 120103, 120104, 120105, 120106, 120107, 120108, 120109, 120110, 120111, (20112) c6( ) c7(Fv, bM, cF, ft, ac, dB, cy, ci, cm, eA); #c1(ADGRB3) c2(XP_005248809) c3(brain-specific angiogenesis inhibitor 3 isoform X1) c4(268587) c5(268588, 268589, 268590, 268591, 268592, 268593) c6( ) c7(Fv, bM, cF, ft, ac, dB, cy, ci, cm, eA); #c1(ADGRB3) c2(XP_011534313) c3(brain-specific angiogenesis inhibitor 3 isoform X5) c4(330681, 330682) c5( ) c6( ) c7(Fv, bM, cF, ft, ac, dB, cy, ci, cm, eA); #c1(ADGRB3) c2(XP_011534315) c3(brain-specific angiogenesis inhibitor 3 isoform X7) c4( ) c5(330683) c6( ) c7(Fv, bM, cF, ft, ac, dB, cy, ci, cm, eA); #c1(ADGRD1) c2(NP_942122) c3(probable G-protein coupled receptor 133 precursor) c4(260466) c5(260467, 260468, 260469, 260470, 260471, 260472) c6( ) c7(EQ, mu); #c1(ADGRD1) c2(XP_011536505) c3(probable G-protein coupled receptor 133 isoform X1) c4(333865) c5(333866, 333867, 333868, 333869, 333870, 333871) c6( ) c7(EQ, mu); #c1(ADGRD1) c2(XP_011536510) c3(probable G-protein coupled receptor 133 isoform X7) c4( ) c5(333872) c6( ) c7(EQ, mu); #c1(ADGRD1) c2(XP_011536511) c3(probable G-protein coupled receptor 133 isoform X8) c4(333873) c5(333874, 333875, 333876, 333877, 333878, 333879) c6( ) c7(EQ, mu); #c1(ADGRD2) c2(XP_006717411) c3(probable G-protein coupled receptor 144) c4(291043, 291044, 291045) c5(291046, 291047, 291048, 291049, 291050, 291051, 291052, 291053, 291054, 291055, 291056, 291057, 291058, 291059, 291060, 291061, 291062, 291063, 291064, 291065, 291066, 291067, 291068, 291069) c6( ) c7(kl); #c1(ADGRE1) c2(NP_001243181) c3(EGF-like module-containing mucin-like hormone receptor-like 1 isoform 2 precursor) c4(90055) c5(90056, 90057, 90058, 90059, 90060, 90061) c6( ) c7(vj, oW, ao, Fw, eh, cd, ad); #c1(ADGRE1) c2(NP_001243182) c3(EGF-like module-containing mucin-like hormone receptor-like 1 isoform 3 precursor) c4(90062) c5(90063, 90064, 90065, 90066, 90067, 90068, 90069, 90070, 90071, 90072) c6( ) c7(vj, oW, ao, Fw, eh, cd, ad); #c1(ADGRE1) c2(NP_001243183) c3(EGF-like module-containing mucin-like hormone receptor-like 1 isoform 4 precursor) c4(90073) c5(90074, 90075, 90076, 90077, 90078, 90079) c6( ) c7(vj, oW, ao, Fw, eh, cd, ad); #c1(ADGRE1) c2(NP_001243184) c3(EGF-like module-containing mucin-like hormone receptor-like 1 isoform 5 precursor) c4(90080) c5(90081, 90082, 90083, 90084, 90085, 90086) c6( ) c7(vj, oW, ao, Fw, eh, cd, ad); #c1(ADGRE1) c2(NP_001965) c3(EGF-like module-containing mucin-like hormone receptor-like 1 isoform 1 precursor) c4(122686) c5(122687, 122688, 122689, 122690, 122691, 122692) c6( ) c7(vj, oW, ao, Fw, eh, cd, ad); #c1(ADGRE2) c2(NP_001257981) c3(EGF-like module-containing mucin-like hormone receptor-like 2 isoform h precursor) c4(95881) c5(95882, 95883, 95884, 95885, 95886, 95887, 95888, 95889, 95890, 95891, 95892, 95893) c6( ) c7(d, q, ci, g); #c1(ADGRE2) c2(NP_038475) c3(EGF-like module-containing mucin-like hormone receptor-like 2 isoform a precursor) c4(176656) c5(176657, 176658, 176659, 176660, 176661, 176662, 176663, 176664, 176665, 176666) c6( ) c7(d, q, ci, g); #c1(ADGRE2) c2(XP_011526251) c3(EGF-like module-containing mucin-like hormone receptor-like 2 isoform X2) c4(320493) c5(320494, 320495, 320496, 320497, 320498, 320499) c6( ) c7(d, q, ci, g); #c1(ADGRE2) c2(XP_011526253) c3(EGF-like module-containing mucin-like hormone receptor-like 2 isoform X4) c4(320500, 320501) c5(320502, 320503, 320504, 320505, 320506, 320507, 320508, 320509, 320510, 320511, 320512, 320513, 320514) c6( ) c7(d, q, ci, g); #c1(ADGRE2) c2(XP_011526254) c3(EGF-like module-containing mucin-like hormone receptor-like 2 isoform X5) c4(320515) c5( ) c6( ) c7(d, q, ci, g); #c1(ADGRE2) c2(XP_011526255) c3(EGF-like module-containing mucin-like hormone receptor-like 2 isoform X6) c4(320516, 320517, 320518) c5(320519, 320520, 320521, 320522, 320523, 320524, 320525, 320526, 320527, 320528, 320529, 320530) c6( ) c7(d, q, ci, g); #c1(ADGRE2) c2(XP_011526257) c3(EGF-like module-containing mucin-like hormone receptor-like 2 isoform X8) c4( ) c5(320531, 320532, 320533, 320534, 320535) c6( ) c7(d, q, ci, g); #c1(ADGRE5) c2(NP_001020331) c3(CD97 antigen isoform 3 preproprotein) c4(28610, 28611) c5(28612, 28613, 28614, 28615, 28616, 28617, 28618, 28619, 28620, 28621, 28622, 28623) c6( ) c7(ai, eG, hd, d, nB, tT, gX, aA, aF, Fx, ci, g, eA, y, q, er, aa); #c1(ADGRE5) c2(NP_001775) c3(CD97 antigen isoform 2 preproprotein) c4(121019) c5(121020, 121021, 121022, 121023, 121024, 121025) c6( ) c7(ai, eG, hd, d, nB, tT, gX, aA, aF, Fx, ci, g, eA, y, q, er, aa); #c1(ADGRE5) c2(NP_510966) c3(CD97 antigen isoform 1 preproprotein) c4(231093) c5(231094, 231095, 231096, 231097, 231098, 231099) c6( ) c7(ai, eG, hd, d, nB, tT, gX, aA, aF, Fx, ci, g, eA, y, q, er, aa); #c1(ADGRF5) c2(NP_056049) c3(probable G-protein coupled receptor 116 precursor) c4(185301, 185302, 185303) c5(185304, 185305, 185306, 185307, 185308, 185309, 185310, 185311, 185312, 185313, 185314, 185315, 185316, 185317, 185318, 185319, 185320, 185321, 185322, 185323, 185324, 185325, 185326, 185327, 185328, 185329, 185330, 185331, 185332, 185333, 185334, 185335) c6( ) c7(gZ, aW, K, aT); #c1(ADGRF5) c2(XP_005248951) c3(probable G-protein coupled receptor 116 isoform X2) c4(268804) c5( ) c6( ) c7(gZ, aW, K, aT); #c1(ADGRF5) c2(XP_011512655) c3(probable G-protein coupled receptor (16 isoform X4) c4( ) c5(303628, 303629, 303630, 303631, 303632, 303633) c6( ) c7(gZ, aW, K, aT); #c1(ADGRF5) c2(XP_011512656) c3(probable G-protein coupled receptor 116 isoform X5) c4(303634) c5( ) c6( ) c7(gZ, aW, K, aT); #c1(ADGRG1) c2(NP_001277071) c3(G-protein coupled receptor 56 isoform d precursor) c4(109004) c5(109005, 109006, 109007, 109008, 109009, 109010) c6( ) c7(T, aC, no, yf, FA, hb, Y, FB, Fz, vj, FD, ff, FC, Fy, en, it, cp); #c1(ADGRG1) c2(NP_001277073) c3(G-protein coupled receptor 56 isoform f) c4(109011) c5( ) c6( ) c7(T, aC, no, yf, FA, hb, Y, FB, Fz, vj, FD, ff, FC, Fy, en, it, cp); #c1(ADGRG1) c2(XP_005256312) c3(G-protein coupled receptor 56 isoform X2) c4(274065) c5(274066, 274067, 274068, 274069, 274070, 274071, 274072, 274073, 274074, 274075, 274076, 274077, 274078, 274079, 274080, 274081, 274082, 274083, 274084, 274085, 274086, 274087, 274088, 274089, 274090, 274091, 274092, 274093) c6( ) c7(T, aC, no, yf, FA, hb, Y, FB, Fz, vj, FD, ff, FC, Fy, en, it, cp); #c1(ADGRG1) c2(XP_006721409) c3(G-protein coupled receptor 56 isoform X1) c4(293979) c5(293980, 293981, 293982, 293983, 293984, 293985, 293986, 293987, 293988, 293989, 293990, 293991, 293992, 293993) c6( ) c7(T, aC, no, yf, FA, hb, Y, FB, Fz, vj, FD, ff, FC, Fy, en, it, cp); #c1(ADGRG2) c2(NP_001073327) c3(G-protein coupled receptor 64 isoform 1 precursor) c4(35852, 35853) c5(35854, 35855, 35856, 35857, 35858) c6( ) c7(af, ci); #c1(ADGRG2) c2(NP_001073328) c3(G-protein coupled receptor 64 isoform 2 precursor) c4(35859) c5(35860, 35861, 35862, 35863, 35864) c6( ) c7(af, ci); #c1(ADGRG2) c2(NP_001073329) c3(G-protein coupled receptor 64 isoform 3 precursor) c4(35865) c5(35866, 35867, 35868, 35869, 35870) c6( ) c7(af, ci); #c1(ADGRG2) c2(NP_001171762) c3(G-protein coupled receptor 64 isoform 5 precursor) c4(71619, 71620) c5(71621, 71622, 71623, 71624, 71625, 71626, 71627, 71628, 71629, 71630) c6( ) c7(af, ci); #c1(ADGRG2) c2(NP_001171763) c3(G-protein coupled receptor 64 isoform 6 precursor) c4(71631) c5(71632, 71633, 71634, 71635, 71636, 71637, 71638, 71639, 71640, 71641, 71642) c6( ) c7(af, ci); #c1(ADGRG2) c2(NP_001171764) c3(G-protein coupled receptor 64 isoform 7 precursor) c4( ) c5(71643, 71644, 71645, 71646, 71647, 71648) c6( ) c7(af, ci); #c1(ADGRG2) c2(NP_001171765) c3(G-protein coupled receptor 64 isoform & precursor) c4(71649) c5(71650, 71651, 71652, 71653, 71654) c6( ) c7(af, ci); #c1(ADGRG2) c2(NP_001171766) c3(G-protein coupled receptor 64 isoform 9 precursor) c4(71655, 71656) c5(71657, 71658, 71659, 71660, 71661) c6( ) c7(af, ci); #c1(ADGRG2) c2(NP_005747) c3(G-protein coupled receptor 64 isoform 4 precursor) c4(158983) c5(158984, 158985, 158986, 158987, 158988) c6( ) c7(af, ci); #c1(ADGRG2) c2(XP_011543735) c3(G-protein coupled receptor 64 isoform X2) c4( ) c5(342787, 342788, 342789, 342790, 342791, 342792, 342793, 342794) c6( ) c7(af, ci); #c1(ADGRG2) c2(XP_011543738) c3(G-protein coupled receptor 64 isoform X3) c4( ) c5(342795) c6( ) c7(af, ci); #c1(ADGRG3) c2(NP_740746) c3(probable G-protein coupled receptor 97 precursor) c4(247437, 247438, 247439) c5(247440, 247441, 247442, 247443, 247444, 247445, 247446, 247447) c6( ) c7(FE, ac); #c1(ADGRG6) c2(NP_001027566) c3(G-protein coupled receptor 126 isoform alpha 2 precursor) c4(30294) c5(30295, 30296, 30297, 30298, 30299, 30300) c6( ) c7(FF, Dp, cF, FG, aa); #c1(ADGRG6) c2(NP_001027567) c3(G-protein coupled receptor 126 isoform beta 2 precursor) c4(30301) c5(30302, 30303, 30304, 30305, 30306, 30307) c6( ) c7(FF, Dp, cF, FG, aa); #c1(ADGRG6) c2(NP_065188) c3(G-protein coupled receptor 126 isoform alpha 1 precursor) c4(204494) c5(204495, 204496, 204497, 204498, 204499, 204500) c6( ) c7(FF, Dp, cF, FG, aa); #c1(ADGRG6) c2(NP_940971) c3(G-protein coupled receptor (26 isoform beta 1 precursor) c4(260216) c5(260217, 260218, 260219, 260220, 260221, 260222) c6( ) c7(FF, Dp, cF, FG, aa); #c1(ADGRG7) c2(NP_116/76) c3(probable G-protein coupled receptor 128 precursor) c4(225614) c5(225615, 225616, 225617, 225618, 225619, 225620) c6( ) c7(jh, nB); #c1(ADGRL1) c2(NP_001008701) c3(latrophilin-1 isoform 1 precursor) c4(23363) c5(23364, 23365, 23366, 23367, 23368, 23369) c6( ) c7(IG, FH, e); #c1(ADGRL1) c2(NP_055736) c3 (latrophilin-1 isoform 2 precursor) c4(183181) c5(183182, 183183, 183184, 183185, 183186, 183187) c6( ) c7(IG, FH, e); #c1(ADGRL3) c2(NP_056051) c3(latrophilin-3 precursor) c4(185370, 185371, 185372) c5(185373, 185374, 185375, 185376, 185377, 185378, 185379, 185380, 185381, 185382, 185383, 185384, 185385, 185386, 185387, 185388, 185389, 185390, 185391, 185392, 185393, 185394, 185395, 185396, 185397) c6( ) c7(IG, cF, FI, K); #c1(ADGRL3) c2(XP_011530086) c3(latrophilin-3 isoform X2) c4(325920) c5( ) c6( ) c7(IG, cF, Fl, K); #c1(ADGRL3) c2(XP_011530087) c3(latrophilin-3 isoform X3) c4(325921, 325922, 325923) c5(325924, 325925, 325926, 325927, 325928, 325929, 325930, 325931, 325932, 325933, 325934, 325935, 325936, 325937, 325938, 325939, 325940, 325941, 325942, 325943, 325944, 325945, 325946, 325947) c6( ) c7(IG, cF, FI, K); #c1(ADGRL3) c2(XP_011530088) c3(latrophilin-3 isoform X4) c4(325948, 325949, 325950, 325951) c5(325952, 325953, 325954, 325955, 325956, 325957, 325958, 325959, 325960, 325961, 325962, 325963, 325964, 325965, 325966, 325967, 325968, 325969, 325970, 325971, 325972, 325973, 325974, 325975) c6( ) c7(IG, cF, FI, K); #c1(ADGRL3) c2(XP_011530092) c3(latrophilin-3 isoform X8) c4(325976) c5(325977, 325978, 325979, 325980, 325981, 325982) c6( ) c7(IG, cF, Fl, K); #c1(ADGRL3) c2(XP_011530093) c3(latrophilin-3 isoform X9) c4(325983) c5(325984, 325985, 325986, 325987, 325988, 325989) c6( ) c7(IG, cF, FI, K); #c1(ADGRL3) c2(XP_011530095) c3(latrophilin-3 isoform X10) c4(325990, 325991, 325992, 325993, 325994) c5(325995, 325996, 325997, 325998, 325999, 326000, 326001, 326002, 326003, 326004, 326005, 326006, 326007, 326008, 326009, 326010, 326011, 326012, 326013, 326014, 326015, 326016, 326017, 326018, 326019, 326020, 326021, 326022, 326023, 326024) c6( ) c7(IG, cF, FI, K); #c1(ADGRV1) c2(NP_115495) c3(G-protein coupled receptor 98 precursor) c4(222901, 222902, 222903) c5(222904, 222905, 222906, 222907, 222908, 222909, 222910, 222911, 222912, 222913, 222914, 222915) c6( ) c7(ha, vr, j, oc, FR, dY, FL, FO, FN, jy, FQ, d, lR, An, FS, nk, nC, op, FK, vA, FP, FJ, FM, Bh); #c1(ADGRV1) c2(XP_011541979) c3(G-protein coupled receptor 98 isoform X3) c4( ) c5(340461) c6( ) c7(ha, vr, j, oc, FR, dY, FL, FO, FN, jy, FQ, d, lR, An, FS, nk, nC, op, FK, vA, FP, FJ, FM, Bh); #c1(ADGRV1) c2(XP_011541981) c3(G-protein coupled receptor 98 isoform X5) c4( ) c5(340462, 340463, 340464, 340465, 340466) c6( ) c7(ha, vr, j, oc, FR, dY, FL, FO, FN, jy, FQ, d, lR, An, FS, nk, nC, op, FK, vA, FP, FJ, FM, Bh); #c1(ADH1A) c2(NP_000658) c3(alcohol dehydrogenase (A) c4(14270) c5(14271, 14272, 14273, 14274, 14275, 14276) c6( ) c7(T, cl, aE, eM, FV, r, aA, FU, na, m, j, an, aF, cm, af, FT); #c1(ADH1B) c2(NP_000659) c3 (alcohol dehydrogenase 18 isoform 1) c4(14277, 14278, 14279) c5(14280, 14281, 14282, 14283, 14284, 14285, 14286, 14287, 14288, 14289, 14290, 14291, 14292) c6( ) c7(Gk, T, uk, fR, e, al, ci, Gt, aE, fS, Gb, Gr, dV, K, hu, aW, mQ, hl, da, Gs, uo, cX, uJ, FW, dD, sQ, aw, ht, aj, FY, Gm, Gw, gl, Go, wb, Gc, hi, ny, FZ, Gh, aq, Gi, hq, ar, ok, Gq, FV, te, ut, jd, it, B, bL, oe, aA, cz, hy, v, j, Ge, q, J, Gj, qp, aT, r, eS, en, iK, FT, ac, Cz, mf, Gg, rA, GI, ob, Gd, bS, eq, fu, uu, Gn, iG, tK, KL, nH, ib, lQ, lR, Gf, Gv, ff, aF, FX, Ga, ek, D, gk, Gu, Gp); #c1(ADH1B) c2(NP_001273579) c3 (alcohol dehydrogenase (B isoform 2) c4( ) c5(105360, 105361, 105362, 105363, 105364) c6( ) c7(Gk, T, uk, fR, e, al, ci, Gt, aE, fS, Gb, Gr, dV, K, hu, aW, mQ, hl, da, Gs, uo, cX, uJ, FW, dD, sQ, aw, ht, aj, FY, Gm, Gw, gl, Go, wb, Gc, hi, ny, FZ, Gh, aq, Gi, hq, ar, ok, Gq, FV, te, ut, jd, it, B, bL, oe, aA, cz, hy, v, j, Ge, q, J, Gj, qp, aT, r, eS, en, iK, FT, ac, Cz, mf, Gg, rA, GI, ob, Gd, bS, eq, fu, uu, Gn, iG, tK, KL, nH, ib, lQ, lR, Gf, Gv, ff, aF, FX, Ga, ek, D, gk, Gu, Gp); #c1(ADH4) c2(NP_000661) c3(alcohol dehydrogenase 4) c4(14293) c5(14294, 14295, 14296, 14297, 14298, 14299) c6( ) c7(ar, T, Gx, FV, ht, an, Gs, fQ, da, Gd, hy, gl, m, Go, hu, iG, t, B, FT); #c1(ADH5) c2(NP_000662) c3(alcohol dehydrogenase class-3) c4(14300) c5(14301, 14302, 14303, 14304, 14305, 14306) c6( ) c7(bM, T, aC, iW, hy, Gn, av, cF, aT, K, Gz, dF, B, Gy, BN, aW, GA, da, hJ, bz, FT, rA, ne); #c1(ADH6) c2(NP_000663) c3(alcohol dehydrogenase 6 isoform 2) c4(14307) c5(14308, 14309, 14310, 14311, 14312, 14313) c6( ) c7(FV, hy, ne, BN, FT); #c1(ADH6) c2(NP_001095940) c3(alcohol dehydrogenase 6 isoform 1) c4(40952, 40953) c5(40954, 40955, 40956, 40957, 40958, 40959, 40960, 40961, 40962, 40963, 40964, 40965) c6( ) c7(FV, hy, ne, BN, FT); #c1(ADH7) c2(NP_000664) c3(alcohol dehydrogenase class 4 mu/sigma chain isoform 2) c4(14314, 14315, 14316) c5(14317, 14318, 14319, 14320, 14321, 14322, 14323, 14324, 14325, 14326, 14327, 14328, 14329, 14330, 14331, 14332, 14333, 14334) c6( ) c7(GI, T, aZ, oe, nx, ht, aA, cz, Gd, aj, hy, j, GC, q, ad, Gw, ae, Gv, ff, r, af, en, cl, iK, fq, Ga, GB, FT, Gs, hL, m, Gu, lB, it); #c1(ADH7) c2(NP_001159976) c3(alcohol dehydrogenase class 4 mu/sigma chain isoform 1) c4(65014) c5(65015, 65016, 65017, 65018, 65019, 65020) c6( ) c7(GI, T, aZ, oe, nx, ht, aA, cz, Gd, aj, hy, j, GC, q, ad, Gw, ae, Gv, ff, r, aF, en, cl, iK, fq, Ga, GB, FT, Gs, hL, m, Gu, lB, it); #c1(ADHFE1) c2(NP_653251) c3(hydroxyacid-oxoacid transhydrogenase, mitochondrial) c4(239343, 239344) c5(239345, 239346, 239347, 239348, 239349, 239350, 239351, 239352, 239353, 239354, 239355, 239356) c6( ) c7(T, s, GD, r, qG, q); #c1(ADI1) c2(NP_060739) c3(1,2-dihydroxy-3-keto-5-methylthiopentene dioxygenase) c4(197555) c5(197556, 197557, 197558, 197559, 197560, 197561) c6( ) c7(aM, dV, t, ae, y, ad); #c1(AD1G) c2(NP_001018092) c3(adipogenin) c4(27312, 27313) c5(27314, 27315, 27316, 27317, 27318, 27319, 27320, 27321, 27322, 27323, 27324, 27325) c6( ) c7(GE); #c1(AD1POQ) c2(NP_00117 (271) c3(adiponectin precursor) c4(70013) c5(70014, 70015, 70016, 70017, 70018, 70019, 70020, 70021, 70022, 70023, 70024, 70025, 70026, 70027) c6( ) c7(T, fR, pf, ea, e, al, bB, aY, ip, GK, aE, Bc, rw, iX, M, K, GO, qM, dQ, hH, wt, aa, aW, fc, hl, g, uC, GR, mx, vy, dD, wh, aw, ht, lN, aj, fr, qN, zP, cd, rc, td, ec, h, lG, jy, GT, uV, A, fQ, aq, bJ, ar, KT, GG, Fi, bo, mg, hl, at, bm, GN, jd, B, bL, jv, eR, oe, qQ, aA, cR, tc, GP, cz, j, fF, C, sl, v, nS, GS, q, J, GL, aT, GQ, cy, bq, Fg, ae, af, kl, wr, iU, fO, GJ, r, iy, GM, cl, sk, ck, wg, ac, hr, fP, mf, dy, bt, aZ, jh, GH, dG, eq, ad, dP, av, nH, F, ts, hD, ji, jo, fb, d, cW, eZ, dq, aF, bz, Gl, qa, bE, aS, cP, ei, GF, hE, y); #c1(ADIPOR1) c2(NP_001277558) c3(adiponectin receptor protein 1) c4(109355, 109356, 109357, 109358) c5(109359, 109360, 109361, 109362, 109363, 109364, 109365, 109366, 109367, 109368, 109369, 109370, 109371, 109372, 109373, 109374, 109375, 109376, 109377, 109378, 109379, 109380, 109381, 109382, 109383, 109384, 109385, 109386) c6( ) c7(bL, T, GV, dD, eR, pf, aZ, ea, e, al, wh, aA, cz, bB, aj, dG, j, ci, v, sl, eb, nS, aY, eq, cd, q, J, ad, rc, F, py, aT, jy, fb, ae, af, kl, d, iU, cW, aW, fc, hl, r, dq, aF, g, cl, fr, GU, bE, go, hl, at, mf, cP, ei, y); #c1(ADIPOR2) c2(XP_006719081) c3(adiponectin receptor protein 2 isoform X1) c4(292203) c5(292204, 292205, 292206, 292207, 292208, 292209) c6( ) c7(bL, T, dD, eR, pf, ea, e, al, aA, bB, tc, aj, v, j, eq, nS, cd, q, J, ad, rc, F, iX, aT, jy, K, bq, fb, ae, af, kl, d, iU, aW, fc, hl, r, aF, g, cl, bE, hL, at, mf, ei, y, ao); #c1(ADIRF) c2(NP_006820) c3(adipogenesis regulatory factor) c4(168386, 168387, 168388) c5(168389, 168390, 168391, 168392, 168393, 168394, 168395, 168396, 168397, 168398, 168399, 168400, 168401, 168402, 168403, 168404, 168405, 168406) c6( ) c7(T, af); #c1(ADK) c2(NP_001114) c3(adenosine kinase isoform a) c4(43361) c5(43362, 43363, 43364, 43365, 43366, 43367) c6( ) c7(T, A, GX, wg, GY, GZ, fO, ha, fd, GW, nA, bq, il, Cr, gD, cB); #c1(ADK) c2(NP_001189378) c3(adenosine kinase isoform c) c4(79305) c5(79306, 79307, 79308, 79309, 79310, 79311) c6( ) c7(T, A, GX, wg, GY, GZ, fO, ha, fd, GW, nA, bq, il, Cr, gD, cB); #c1(ADK) c2(NP_001189379) c3 (adenosine kinase isoform d) c4(79312) c5(79313, 79314, 79315, 79316, 79317, 79318) c6( ) c7(T, A, GX, wg, GY, GZ, fO, ha, fd, GW, nA, bq, il, Cr, gD, cB); #c1(ADK) c2(NP_006712) c3(adenosine kinase isoform b) c4(167342) c5(167343, 167344, 167345, 167346, 167347, 167348) c6( ) c7(T, A, GX, wg, GY, GZ, fO, ha, fd, GW, nA, bq, il, Cr, gD, cB); #c1(ADM) c2(NP_001115) c3(ADM precursor) c4(43368) c5(43369, 43370, 43371, 43372, 43373, 43374, 43375, 43376, 43377, 43378, 43379, 43380, 43381, 43382) c6( ) c7(bM, T, aC, fR, ea, if, e, eb, bB, fd, zh, ci, aY, Hr, aE, bW, M, iv, K, wt, dQ, eL, hm, Hd, DA, aW, g, as, eF, cm, uC, y, iT, ki, uo, Hb, m, hh, fy, Ht, dD, uH, Hm, aw, qy, aj, Ea, qN, qW, cd, BI, Hf, jL, lG, dL, ey, ux, vx, Ha, A, lb, fQ, mo, W, dB, hk, lf, hl, at, sa, B, bL, ka, dA, AL, Hj, oe, He, cz, tc, v, j, C, GS, q, J, eA, eJ, ns, rS, aT, cy, bK, bq, ae, dl, Hi, r, en, cl, t, bN, ck, ac, wl, hc, fP, mf, dy, bt, Hc, Hn, OR, dG, eq, Fc, ad, dZ, Ho, av, F, Hk, vc, hP, ji, nJ, vP, uX, d, Hq, Hh, ff, eH, zR, eZ, uF, HI, bz, qa, ek, Hp, Ew, bE, pP, gZ, Hs, aS, Hg, ei, lC, X, AG); #c1(ADM2) c2(NP_001240774) c3(ADM2 preproprotein) c4(88777) c5(88778, 88779, 88780, 88781, 88782, 88783, 88784, 88785, 88786, 88787, 88788, 88789, 88790, 88791, 88792, 88793, 88794, 88795, 88796) c6(88797, 88798, 88799, 88800) c7(T, B, tc, Hu, aS, j, eE, cd); #c1(ADNP) c2(XP_011527049) c3 (activity-dependent neuroprotector homeobox protein isoform X1) c4(321365) c5(321366, 321367, 321368, 321369, 321370, 321371, 321372) c6(321373, 321374) c7(Hx, Hw, wg, cy, nA, xg, Hv, bN); #c1(ADNP) c2(XP_011527050) c3 (activity-dependent neuroprotector homeobox protein isoform X2) c4(321375) c5(321376, 321377, 321378, 321379, 321380, 321381, 321382, 321383, 321384, 321385, 321386, 321387, 321388, 321389, 321390, 321391, 321392, 321393, 321394, 321395, 321396, 321397, 321398, 321399, 321400, 321401, 321402, 321403, 321404, 321405, 321406, 321407, 321408, 321409, 321410, 321411, 321412, 321413, 321414, 321415, 321416, 321417, 321418, 321419, 321420, 321421, 321422, 321423, 321424, 321425, 321426, 321427, 321428, 321429, 321430, 321431, 321432, 321433, 321434, 321435, 321436, 321437, 321438, 321439, 321440, 321441, 321442, 321443, 321444, 321445, 321446, 321447, 321448, 321449, 321450, 321451, 321452, 321453, 321454, 321455, 321456, 321457, 321458, 321459, 321460, 321461, 321462, 321463, 321464, 321465, 321466, 321467, 321468, 321469, 321470, 321471, 321472, 321473, 321474, 321475, 321476, 321477, 321478, 321479, 321480, 321481, 321482, 321483, 321484, 321485, 321486, 321487, 321488, 321489, 321490) c6(321491, 321492, 321493, 321494, 321495, 321496, 321497, 321498, 321499) c7(Hx, Hw, wg, cy, nA, xg, Hv, bN); #c1(ADO) c2(NP_116193) c3(2-aminoethanethiol dioxygenase) c4(225673) c5(225674, 225675, 225676, 225677, 225678, 225679) c6( ) c7(zw, zs, Hz, HA, HB, Hy, ji, dr); #c1(ADORA1) c2(NP_000665) c3(adenosine receptor A1) c4(14335, 14336) c5(14337, 14338, 14339, 14340, 14341, 14342, 14343, 14344, 14345, 14346, 14347, 14348, 14349, 14350, 14351, 14352, 14353, 14354, 14355) c6( ) c7(bM, FW, wi, dA, fV, aw, cz, aj, v, j, ww, J, cc, fS, HC, aT, cy, bK, K, bq, HD, Cr, aa, A, iU, aW, vs, qa, ek, hq, bb, mx); #c1(ADORA1) c2(XP_005244959) c3(adenosine receptor A1 isoform X1) c4( ) c5(266166) c6( ) c7(bM, FW, wi, dA, fV, aw, cz, aj, v, j, ww, J, cc, fS, HC, aT, cy, bk, K, bq, HD, Cr, aa, A, iU, aW, vs, qa, ek, hq, bb, mx); #c1(ADORA2A) c2(NP_001265429) c3(adenosine receptor A2a) c4(98540, 98541, 98542, 98543, 98544) c5(98545, 98546, 98547, 98548, 98549, 98550, 98551, 98552, 98553, 98554, 98555, 98556, 98557, 98558, 98559, 98560, 98561, 98562, 98563, 98564, 98565, 98566, 98567, 98568, 98569, 98570, 98571, 98572, 98573, 98574, 98575, 98576, 98577, 98578, 98579, 98580, 98581, 98582) c6( ) c7(bM, dD, fV, ex, cz, HE, ur, uu, eq, ww, HH, cd, HF, cc, w, HJ, hb, fS, HG, lG, cy, eV, bq, an, Cr, xa, aa, DA, tb, Di, HI, fQ, HL, wA, pb, qa, lx, hq, bN, pa, rR, cA, mx, Be, HK, at, sv, y, cB); #c1(ADORA2B) c2(NP_000667) c3(adenosine receptor A2b) c4(14356) c5(14357, 14358, 14359, 14360, 14361, 14362) c6( ) c7(bL, bM, T, dD, qb, cz, bB, aj, dG, j, eq, q, J, fS, aT, cy, K, s, Y, fe, g, uC, bE, mg, at, fD, lC); #c1(ADPGK) c2(NP_112574) c3(ADP-dependent glucokinase precursor) c4(221449, 221450) c5(221451, 221452, 221453, 221454, 221455, 221456, 221457, 221458, 221459, 221460, 221461, 221462, 221463) c6( ) c7(Ea); #c1(ADPGK) c2(XP_006720775) c3(ADP-dependent glucokinase isoform X1) c4(293679) c5(293680, 293681, 293682, 293683, 293684, 293685, 293686) c6( ) c7(Ea); #c1(ADPGK) c2(XP_006720776) c3(ADP-dependent glucokinase isoform X2) c4(293687) c5(293688, 293689, 293690, 293691, 293692) c6( ) c7(Ea); #c1(ADPRH) c2(NP_001278878) c3(ADP-ribosylarginine hydrolase isoform 1) c4(110750, 110751) c5(110752, 110753, 110754, 110755, 110756, 110757, 110758, 110759, 110760, 110761, 110762, 110763, 110764, 110765, 110766, 110767, 110768, 110769, 110770, 110771, 110772, 110773, 110774, 110775) c6( ) c7(cH, HM); #c1(ADPRHL1) c2(NP_612439) c3([Protein ADP-ribosylarginine] hydrolase-like protein 1 isoform 1) c4(236505, 236506, 236507) c5(236508, 236509, 236510, 236511, 236512, 236513, 236514, 236515) c6( ) c7(HN); #c1(ADPRHL1) c2(NP_954631) c3([Protein ADP-ribosylarginine] hydrolase-like protein 1 isoform 2) c4( ) c5(261093, 261094, 261095, 261096, 261097, 261098, 261099, 261100, 261101, 261102) c6( ) c7(HN); #c1(ADPRHL2) c2(XP_011539938) c3(poly(ADP-ribose) glycohydrolase ARH3 isoform X1) c4(338296) c5(338297, 338298, 338299, 338300, 338301) c6( ) c7(aT); #c1(ADRA1A) c2(NP_000671) c3(alpha-1A adrenergic receptor isoform 1) c4(14377) c5(14378, 14379, 14380, 14381, 14382, 14383, 14384, 14385, 14386, 14387) c6( ) c7(bL, bM, G, T, HQ, qX, vS, iH, eR, yk, oe, ha, e, EQ, cz, hx, al, hU, v, j, fd, yp, xY, ye, cd, ad, eA, yn, av, ot, ec, DK, gH, nX, xW, sy, aT, h, xV, bK, K, yb, bq, yh, HR, yj, ae, af, HO, an, yi, xX, d, xU, kl, aW, sp, Y, yL, yc, yq, t, nC, bz, iK, qa, iP, cv, jq, dR, D, ok, yf, bP, yl, xZ, HP, cj, tl, m, ei, y, B); #c1(ADRA1A) c2(NP_150645) c3(alpha-1A adrenergic receptor isoform 3) c4(228225) c5(228226, 228227, 228228, 228229, 228230, 228231, 228232, 228233, 228234, 228235, 228236) c6( ) c7(bL, bM, G, T, HQ, qX, vS, iH, eR, yk, oe, ha, e, EQ, cz, hx, al, hU, v, j, fd, yp, xY, ye, cd, ad, eA, yn, av, ot, ec, DK, gH, nX, xW, sy, aT, h, xV, bK, K, yb, bq, yh, HR, yj, ae, af, HO, an, yi, xX, d, xU, kl, aW, sp, Y, yL, yc, yq, t, nC, bz, ik, qa, iP, cv, jq, dR, D, ok, yf, bP, yl, xZ, HP, cj, tl, m, ei, y, B); #c1(ADRA1A) c2(NP_150646) c3(alpha-1A adrenergic receptor isoform 2) c4(228237, 228238, 228239) c5(228240, 228241, 228242, 228243, 228244, 228245, 228246) c6( ) c7(bL, bM, G, T, HQ, qX, vS, iH, eR, yk, oe, ha, e, EQ, cz, hx, al, hU, v, j, fd, yp, xY, ye, cd, ad, eA, yn, av, ot, ec, DK, gH, nX, xW, sy, aT, h, xV, bK, K, yb, bq, yh, HR, yj, ae, af, HO, an, yi, xx, d, xU, kl, aW, sp, Y, yL, yc, yq, t, nC, bz, iK, qa, iP, cv, jq, dR, D, ok, yf, bP, yl, xZ, HP, cj, tl, m, ei, y, B); #c1(ADRA1A) c2(NP_150647) c3(alpha-LA adrenergic receptor isoform 4) c4(228247) c5(228248, 228249, 228250, 228251, 228252, 228253) c6( ) c7(bL, bM, G, T, HQ, qX, vS, iH, eR, yk, oe, ha, e, EQ, cz, hx, al, hU, v, j, fd, yp, xY, ye, cd, ad, eA, yn, av, ot, ec, DK, gH, nX, xW, sy, aT, h, xV, bK, K, yb, bq, yh, HR, yj, ae, af, HO, an, yi, xX, d, xU, kl, aW, sp, Y, yL, yc, yq, t, nC, bz, ik, qa, iP, cv, jq, dR, D, ok, yf, bP, yl, xZ, HP, cj, tl, m, ei, y, B); #c1(ADRA1A) c2(XP_011542713) c3(alpha-1A adrenergic receptor isoform X4) c4(34/261) c5(341262, 34/263, 341264, 341265, 341266, 341267, 341268, 341269, 341270, 341271, 341272, 341273) c6( ) c7(bL, bM, G, T, HQ, qX, vS, iH, eR, yk, oe, ha, e, EQ, cz, hx, al, hU, v, j, fd, yp, xY, ye, cd, ad, eA, yn, av, ot, ec, DK, gH, nX, xW, sy, aT, h, xV, bK, K, yb, bq, yh, HR, yj, ae, af, HO, an, yi, xX, d, xU, kl, aW, sp, Y, yL, yc, yq, t, nC, bz, iK, qa, iP, cv, jq, dR, D, ok, yf, bP, yl, xZ, HP, cj, tl, m, ei, y, B); #c1(ADRA1A) c2(XP_011542714) c3(alpha-1A adrenergic receptor isoform X5) c4(341274, 341275) c5(341276, 341277, 341278, 341279, 341280, 341281, 341282, 341283, 341284, 341285, 341286, 341287) c6( ) c7(bL, bM, G, T, HQ, qX, vS, iH, eR, yk, oe, ha, e, EQ, cz, hx, al, hU, v, j, fd, yp, xY, ye, cd, ad, eA, yn, av, ot, ec, DK, gH, nX, xW, sy, aT, h, xV, bK, K, yb, bq, yh, HR, yj, ae, af, HO, an, yi, xX, d, xU, kl, aW, sp, Y, yL, yc, yq, t, nC, bz, ik, qa, iP, cv, jq, dR, D, ok, yf, bP, yl, xZ, HP, cj, tl, m, ei, y, B); #c1(ADRA1B) c2(NP_000670) c3(alpha-1B adrenergic receptor) c4(14370) c5(14371, 14372, 14373, 14374, 14375, 14376) c6( ) c7(bM, kl, eR, cA, oe, hx, HC, r, aA, cz, v, dy, K, bK, aF, q, qa, iq); #c1(ADRA1D) c2(NP_000669) c3(alpha-1D adrenergic receptor) c4(14363) c5(14364, 14365, 14366, 14367, 14368, 14369) c6( ) c7(bM, T, uk, lP, ea, al, lL, bB, cL, zh, HS, ci, ur, lK, lN, HU, lR, aR, lS, lF, bv, lE, HV, aE, lu, dv, HR, K, lQ, N, lZ, UM, KF, lB, lh, aW, lW, lh, az, hl, ih, g, as, eF, cm, lk, DL, ly, lt, lq, mx, cj, wx, m, lc, cZ, dD, lG, lT, lo, aw, cV, lb, n, aj, BF, ld, cd, tD, sd, h, jy, HX, ey, pj, dd, A, HY, aq, Hu, xe, HT, lJ, vU, iO, bP, cA, lX, ln, Be, hR, ER, lA, cB, B, lf, L, ll, lp, fM, eR, fG, hx, jx, DV, aA, lj, cz, j, C, Eh, pd, q, J, o, lO, mj, hW, lg, aT, zp, kU, lM, ae, af, lr, bA, hM, kl, s, rh, HZ, Y, yL, ev, lC, r, lH, ls, bQ, t, qs, ik, pb, lv, vk, lY, ck, sS, BU, c, kB, ll, lV, HW, hr, wq, dy, EY, lw, gT, Fl, ao, am, kh, id, ob, ha, li, ID, w, bS, uu, jE, dG, ah, eq, tK, ad, hX, F, le, oj, oW, ab, vw, lm, ji, lz, d, lx, lU, Fz, eH, eZ, hp, la, S, uF, Z, iV, bz, dr, Ek, qa, dR, gk, DT, qe, mi, bE, dJ, aS, ei, y, fh, wS, us); #c1(ADRA2A) c2(NP_000672) c3(alpha-2A adrenergic receptor) c4(14388) c5(14389, 14390, 14391, 14392, 14393, 14394) c6( ) c7(bL, HQ, oe, ha, al, bO, cz, jh, BH, Ja, j, ct, C, bS, Jd, aj, v, ww, J, xt, mj, l, DK, Jb, Fo, io, K, jo, qM, hB, an, ae, af, Jc, yL, gC, iV, Z, bJ, hq, cA, HP, gZ, qq, ei, ra, Fl, Je); #c1(ADRA2B) c2(NP_000673) c3(alpha-28 adrenergic receptor) c4(14395) c5(14396, 14397, 14398, 14399, 14400, 14401) c6( ) c7(bL, bM, T, dD, HQ, iH, ea, yk, oe, ha, e, EQ, cz, hU, aw, aj, v, j, eq, nA, yp, fd, Jf, ye, xY, J, cd, ad, eA, yn, av, uT, xW, aT, h, xV, bK, K, yb, yh, yj, ae, hm, aa, yi, xX, d, xU, kl, aW, Y, yc, yq, t, nC, bz, ik, qa, iP, bJ, jq, dR, ok, yf, pO, yl, bP, xZ, Ei, cj, tl, mf, m, ei, y, gq); #c1(ADRA2C) c2(NP_000674) c3(alpha-2C adrenergic receptor) c4(14402) c5(14403, 14404, 14405, 14406, 14407, 14408) c6( ) c7(bL, HQ, oe, bO, cz, bS, uj, ct, cd, xt, DK, he, mu, Jj, Jg, Z, jb, bN, Jh, cA, HP, Ji, mx, sv, ra); #c1(ADRB1) c2(NP_000675) c3(beta- adrenergic receptor) c4(14409) c5(14410, 14411, 14412, 14413, 14414, 14415) c6( ) c7(HQ, T, dD, eR, mr, ea, wL, oe, e, aw, EQ, aA, cz, tc, aj, dG, j, v, vp, bO, eq, uW, Cb, ye, cd, Jm, cc, ec, Bc, lC, HD, lG, Jn, K, qM, bq, da, vP, af, hm, mu, A, fQ, pD, mo, Jk, JI, dw, qa, bJ, ac, pZ, cA, mx, ll, gZ, uz, at, pK, fy, iu, rk); #c1(ADRB2) c2(NP_000015) c3(beta-2 adrenergic receptor) c4(69) c5(70, 71, 72, 73, 74, 75) c6( ) c7(bM, T, aC, vS, JN, FR, Js, pf, ea, e, al, EQ, fd, cl, gf, lN, Jx, Jt, aY, aR, x, aE, Bc, JD, Gb, M, Ds, K, qM, dQ, eN, aa, dH, JI, aW, mQ, fc, JF, pD, g, eE, cm, uC, Jr, JA, Jp, mx, U, cj, uo, wx, k, gn, FW, dD, sQ, JC, JE, aw, aj, ct, pM, cd, JB, cc, ec, Jv, cU, lG, jy, Jn, h, A, sp, xi, fQ, po, dw, CM, fq, bJ, kT, GG, cA, cO, bo, JM, at, E, Dp, ut, eo, JJ, sZ, B, Jo, bL, Dw, jv, wj, eR, DD, wL, oe, Ju, qQ, uv, aA, cz, tc, v, j, C, bX, uW, V, eA, J, o, mP, lg, Jw, nX, aT, cy, bK, sR, af, eu, hM, kl, Y, r, en, f, bN, ck, JG, cT, ac, c, pZ, ll, hr, uS, JL, mf, dy, JH, fD, gT, JO, ao, HQ, sY, qX, qb, aZ, iW, uG, Jy, OR, wV, bS, Jz, uu, eq, Fb, ad, Jm, fU, av, Jq, hD, JK, lQ, sj, mu, gj, lR, cH, ff, dq, aF, un, iV, Z, bz, qa, DF, D, gk, z, ag, uR, ei, ol, pk, y, iu, rk); #c1(ADRB3) c2(NP_000016) c3(beta-3 adrenergic receptor) c4(76) c5(77, 78, 79, 80, 81, 82) c6( ) c7(T, e, bO, bB, lN, st, GK, wM, u, iX, qM, dQ, aW, fc, hl, uC, ba, vy, wx, aw, aj, cd, cc, ec, jy, uV, vx, A, ES, fQ, dw, cM, fq, JR, JP, bJ, KT, GG, cA, JA, mg, hl, at, bl, eR, wL, oe, qQ, cz, v, j, nS, J, jF, nX, aT, cy, bq, af, dl, kl, bg, r, cl, c, ll, JL, mf, HQ, qX, aZ, iW, uG, dG, tK, Jm, nH, l, ji, JS, sj, eZ, dq, qa, bE, ei, y, vt, X); #c1(ADRBK1) c2(NP_001610) c3(beta-adrenergic receptor kinase 1) c4(119185) c5(119186, 119187, 119188, 119189, 119190, 119191, 119192) c6( ) c7(T, JC, aw, uG, cz, cR, tc, aj, dG, j, ci, v, Cb, JW, cd, aR, J, ad, Hx, hd, cc, oz, JT, aT, cy, bK, K, yb, an, ae, af, ak, yo, JU, s, aW, Y, JV, fJ, fQ, dq, aq, iR, cm, ac, nB, xZ, mx, ll, tV, gZ, hR, Da, lC, B, y); #c1(ADRBK2) c2(NP_005151) c3(beta-adrenergic receptor kinase 2) c4(153430, 153431) c5(153432, 153433, 153434, 153435, 153436, 153437, 153438, 153439, 153440, 153441, 153442, 153443) c6( ) c7(gV, ai, JX, d, bP, hG, mx, ll, gZ, he, bk, K, an, gT, cd); #c1(ADRM1) c2(XP_011526804) c3(proteasomal ubiquitin receptor ADRM1 isoform X1) c4(321101, 321102) c5( ) c6( ) c7(T, y, aW, aT, aA, n, h, af, ci, m, t, ae, ad); #c1(ADRM1) c2(XP_011526805) c3(proteasomal ubiquitin receptor ADRM1 isoform X2) c4( ) c5(321103, 321104, 321105, 321106, 321107, 321108, 321109, 321110, 321111, 321112, 321113, 321114) c6( ) c7(T, y, aW, aT, aA, n, h, af, ci, m, t, ae, ad); #c1(ADSL) c2(NP_000017) c3(adenylosuccinate lyase isoform a) c4(83, 84) c5(85, 86, 87, 88, 89, 90, 91, 92, 93, 94, 95, 96) c6( ) c7(T, mO, d, JZ, jp, JY, aT, dq, Ka, aq, K, nA, bq, aR, bN); #c1(ADSL) c2(NP_001116850) c3(adenylosuccinate lyase isoform b) c4(43993) c5(43994, 43995, 43996, 43997, 43998, 43999, 44000, 44001, 44002, 44003, 44004, 44005) c6( ) c7(T, mO, d, JZ, jp, JY, aT, dq, Ka, aq, K, nA, bq, aR, bN); #c1(ADSL) c2(XP_011528281) c3(adenylosuccinate lyase isoform X4) c4(323354) c5( ) c6( ) c7(T, mO, d, JZ, jp, JY, aT, dq, Ka, aq, K, nA, bq, aR, bN); #c1(ADSS) c2(NP_001117) c3 (adenylosuccinate synthetase isozyme 2) c4(44033, 44034) c5(44035, 44036, 44037, 44038, 44039, 44040, 44041, 44042, 44043, 44044, 44045, 44046) c6( ) c7(eE, lo, ey, ac, Kb); #c1(ADSSL1) c2(NP_689541) c3(adenylosuccinate synthetase isozyme 1 isoform 2) c4(243041) c5(243042, 243043, 243044, 243045, 243046, 243047) c6( ) c7(bz, bq); #c1(ADSSL1) c2(NP_954634) c3(adenylosuccinate synthetase isozyme 1 isoform 1) c4(261103) c5(261104, 261105, 261106, 261107, 261108, 261109) c6( ) c7(bz, bq); #c1(ADTRP) c2(NP_001137420) c3 (androgen-dependent TFPI-regulating protein isoform 1) c4(55672) c5(55673, 55674, 55675, 55676, 55677, 55678) c6( ) c7(cd, j); #c1(ADTRP) c2(NP_116133) c3(androgen-dependent TFPI-regulating protein isoform 2) c4(225465) c5(225466, 225467, 225468, 225469, 225470, 225471) c6( ) c7(cd, j); #c1(ADTRP) c2(XP_011513258) c3(androgen-dependent TFPI-regulating protein isoform X2) c4(305503) c5(305504, 305505, 305506, 305507, 305508, 305509) c6( ) c7(cd, j); #c1(ADTRP) c2(XP_011513260) c3(androgen-dependent TFPI-regulating protein isoform X3) c4(305510) c5( ) c6( ) c7(cd, j); #c1(ADTRP) c2(XP_011513261) c3(androgen-dependent TFPI-regulating protein isoform X4) c4(305511) c5(305512, 305513, 305514, 305515, 305516, 305517, 305518, 305519, 305520, 305521, 305522, 305523) c6( ) c7(cd, j); #c1(AEBP2) c2(NP_001107648) c3(zinc finger protein AEBP2 isoform b) c4(42946, 42947, 42948, 42949) c5(42950, 42951, 42952, 42953, 42954, 42955, 42956, 42957, 42958) c6( ) c7(Kc); #c1(AEBP2) c2(NP_001253972) c3(zinc finger protein AEBP2 isoform c) c4( ) c5(94335, 94336, 94337, 94338, 94339, 94340) c6( ) c7(Kc); #c1(AEBP2) c2(NP_694939) c3(zinc finger protein AEBP2 isoform a) c4( ) c5(245434, 245435, 245436, 245437, 245438, 245439, 245440, 245441, 245442, 245443) c6( ) c7(Kc); #c1(AEN) c2(NP_073604) c3(apoptosis-enhancing nuclease) c4(213037, 213038) c5(213039, 213040, 213041, 213042, 213043, 213044, 213045, 213046, 213047, 213048, 213049, 213050) c6( ) c7(af); #c1(AES) c2(NP_945320) c3(amino-terminal enhancer of split isoform a) c4(260811) c5(260812, 260813, 260814, 260815, 260816, 260817) c6( ) c7(Kd, Ke, j, yb, bt, iu); #c1(AFAP1) c2(NP_001128119) c3(actin filament-associated protein 1 isoform A) c4( ) c5(49551, 49552, 49553, 49554, 49555, 49556) c6( ) c7(bF, cZ, dH, aW, aT, r, Kf, ci, K, dd, ae, ad); #c1(AFAP1) c2(XP_006713971) c3(actin filament-associated protein 1 isoform X2) c4(287464) c5( ) c6( ) c7(bF, cZ, dH, aW, aT, r, Kf, ci, K, dd, ae, ad); #c1(AFAP1) c2(XP_006713972) c3(actin filament-associated protein 1 isoform X4) c4(287465) c5(287466, 287467, 287468, 287469, 287470, 287471, 287472, 287473, 287474, 287475) c6( ) c7(bF, cZ, dH, aW, aT, r, Kf, ci, K, dd, ae, ad); #c1(AFAP1L1) c2(NP_001139809) c3(actin filament-associated protein 1-like 1 isoform 2) c4(59990) c5(59991, 59992, 59993, 59994, 59995, 59996) c6( ) c7(Kg); #c1(AFAP1L1) c2(NP_689619) c3(actin filament-associated protein 1-like 1 isoform 1) c4(243310) c5(243311, 243312, 243313, 243314, 243315, 243316) c6( ) c7(Kg); #c1(AFAP1L2) c2(NP_001001936) c3(actin filament-associated protein 1-like 2 isoform 1) c4(19178) c5(19179, 19180, 19181, 19182, 19183, 19184) c6( ) c7(T, hd, nB, aA, it, n, aF, bz); #c1(AFAP1L2) c2(NP_001274753) c3(actin filament-associated protein 1-like 2 isoform 3) c4(106773) c5(106774, 106775, 106776, 106777, 106778, 106779) c6( ) c7(T, hd, nB, aA, it, n, aF, bz); #c1(AFAP1L2) c2(NP_115939) c3(actin filament-associated protein 1-like 2 isoform 2) c4(224794, 224795) c5(224796, 224797, 224798, 224799, 224800, 224801, 224802, 224803, 224804, 224805, 224806, 224807) c6( ) c7(T, hd, nB, aA, it, n, aF, bz); #c1(AFAP1L2) c2(XP_005270290) c3(actin filament-associated protein 1-like 2 isoform X9) c4(283322) c5(283323, 283324, 283325, 283326, 283327, 283328, 283329, 283330, 283331, 283332) c6( ) c7(T, hd, nB, aA, it, n, aF, bz); #c1(AFF1) c2(NP_001160165) c3(AF4/FMR2 family member 1 isoform 1) c4(65171, 65172) c5(65173) c6( ) c7(ck, x, T, ex, eZ, eV, j, k, fl, hF, bt, mC); #c1(AFF1) c2(NP_005926) c3(AF4/FMR2 family member 1 isoform 2) c4(160486) c5(160487, 160488, 160489, 160490, 160491, 160492, 160493) c6( ) c7(ck, x, T, ex, eZ, eV, j, k, fl, hF, bt, mC); #c1(AFF1) c2(XP_005263066) c3(AF4/FMR2 family member 1 isoform X2) c4(278301, 278302) c5( ) c6( ) c7(ck, x, T, ex, eZ, eV, j, k, fL, hF, bt, mC); #c1(AFF1) c2(XP_005263070) c3(AF4/FMR2 family member 1 isoform X5) c4( ) c5(278303, 278304, 278305, 278306, 278307, 278308, 278309, 278310, 278311, 278312, 278313, 278314, 278315, 278316, 278317, 278318, 278319) c6( ) c7(ck, x, T, ex, eZ, eV, j, k, fL, hF, bt, mC); #c1(AFF2) c2(NP_001162593) c3(AF4/FMR2 family member 2 isoform 2) c4(66435, 66436) c5(66437, 66438, 66439, 66440, 66441, 66442, 66443, 66444, 66445, 66446, 66447, 66448, 66449, 66450, 66451) c6( ) c7(oe, Kk, Kn, Kh, Kw, nA, hF, Kx, aR, xt, x, Ks, il, nd, yZ, Ko, rz, Kq, Kt, nf, Ku, zZ, Kp, Kl, dR, ck, Ki, Kv, Kr, Km, Kj); #c1(AFF2) c2(NP_001162596) c3(AF4/FMR2 family member 2 isoform 5) c4(66452) c5(66453, 66454, 66455, 66456, 66457, 66458) c6( ) c7(oe, Kk, Kn, Kh, Kw, nA, hF, Kx, aR, xt, x, Ks, il, nd, yZ, Ko, rz, Kq, Kt, nf, Ku, zZ, Kp, Kl, dR, ck, Ki, Kv, Kr, Km, Kj); #c1(AFF2) c2(NP_001164099) c3(AF4/FMR2 family member 2 isoform 6) c4(66584) c5(66585, 66586, 66587, 66588, 66589, 66590) c6( ) c7(De, Kk, Kn, Kh, Kw, nA, hF, Kx, aR, xt, x, Ks, il, nd, yZ, Ko, rz, Kq, Kt, nf, Ku, zZ, Kp, Kl, dR, ck, Ki, Kv, Kr, Km, Kj); #c1(AFF2) c2(NP_002016) c3(AF4/FMR2 family member 2 isoform 1) c4(123324) c5(123325, 123326, 123327, 123328, 123329, 123330, 123331, 123332, 123333) c6( ) c7(oe, Kk, Kn, Kh, Kw, nA, hF, Kx, aR, xt, x, Ks, il, nd, yZ, Ko, rz, Kq, Kt, nf, Ku, zZ, Kp, Kl, dR, ck, Ki, Kv, Kr, Km, Kj); #c1(AFF3) c2(NP_001020279) c3(AF4/FMR2 family member 3 isoform 2) c4(28535) c5(28536, 28537, 28538, 28539, 28540, 28541, 28542, 28543, 28544, 28545) c6( ) c7(T, eR, IB, oe, cL, Ea, nA, hF, aY, o, wD, x, aT, A, aW, ck, ou, iT, Jr, k, y, bt); #c1(AFF3) c2(NP_002276) c3(AF4/FMR2 family member 3 isoform 1) c4(127171, 127172, 127173, 127174, 127175, 127176, 127177, 127178) c5(127179, 127180, 127181, 127182, 127183, 127184, 127185, 127186, 127187, 127188, 127189, 127190, 127191, 127192, 127193, 127194, 127195, 127196, 127197, 127198, 127199, 127200, 127201, 127202, 127203, 127204, 127205, 127206, 127207, 127208, 127209, 127210, 127211, 127212, 127213, 127214, 127215, 127216, 127217, 127218, 127219, 127220, 127221) c6( ) c7(T, eR, lB, oe, cL, Ea, nA, hF, aY, o, wD, x, aT, A, aW, ck, ou, iT, Jr, k, y, bt); #c1(AFF3) c2(XP_011509471) c3(AF4/FMR2 family member 3 isoform X3) c4(300046) c5( ) c6( ) c7(T, eR, lB, oe, cL, Ea, nA, hF, aY, o, wD, x, aT, A, aW, ck, ou, iT, Jr, k, y, bt); #c1(AFF3) c2(XP_011509472) c3(AF4/FMR2 family member 3 isoform X4) c4(300047) c5(300048, 300049, 300050, 300051, 300052, 300053, 300054, 300055, 300056, 300057, 300058, 300059, 300060, 300061, 300062, 300063, 300064) c6( ) c7(T, eR, lB, oe, cL, Ea, nA, hF, aY, o, wD, x, aT, A, aW, ck, ou, iT, Jr, k, y, bt); #c1(AFF4) c2(NP_055238) c3(AF4/FMR2 family member 4) c4(179996) c5(179997, 179998, 179999, 180000, 180001, 180002, 180003) c6( ) c7(ck, x, ou, T, h, hn, k); #c1(AFF4) c2(XP_006714650) c3(AF4/FMR2 family member 4 isoform X2) c4(287933) c5(287934, 287935, 287936, 287937, 287938, 287939) c6( ) c7(ck, x, ou, T, h, hn, k); #c1(AFG3L2) c2(NP_006787) c3(AFG3-like protein 2) c4(168069, 168070) c5(168071, 168072, 168073, 168074, 168075, 168076, 168077, 168078, 168079, 168080, 168081, 168082) c6( ) c7(KC, KG, yr, KE, KB, ky, lu, KD, KB, aV, Ky, aN, U, Kz, KA, yZ, rf, fu, yU, KF); #c1(AFM) c2(NP_001124) c3(afamin precursor) c4( ) c5( ) c6( ) c7(T, Ki, jx, hl, aq, m, nA, t, B, hX); #c1(AFP) c2(NP_001125) c3(alpha-fetoprotein precursor) c4(49207) c5(49208, 49209, 49210, 49211, 49212, 49213) c6( ) c7(T, KK, al, bB, ci, aE, pX, KP, K, JI, aW, hl, KX, La, g, DL, lp, dc, m, gn, Le, wu, fg, n, aj, zP, eW, KL, ft, jz, KN, KV, Df, Lg, Ck, KH, Lh, Ff, fQ, aq, LI, cM, Ld, iO, ky, eD, bo, KT, B, Kl, jp, KR, jx, aA, bX, aT, rO, s, vT, Lf, lo, Lc, bu, f, KQ, xw, vW, Li, KQ, KA, dy, KY, ao, vi, ml, KW, jh, Hn, KO, jE, fu, hX, F, tT, wm, KU, CH, KS, Lk, aF, xH, Fq, KJ, iR, KM, dr, bF, gk, mi, bE, Lj, KZ, Lb, bC, be, hE, KF); #c1(AGA) c2(NP_000018) c3(N(4)-(beta-N-acetylglucosaminyl)-L-asparaginase isoform 1 preproprotein) c4(97) c5(98, 99, 100, 101, 102, 103) c6( ) c7(bM, aA, ad, Lr, dv, h, nm, jy, ae, mR, Ln, Lm, aF, hp, cl, ac, of, hL, Lp, hO, Lq, Lo); #c1(AGAP1) c2(NP_001032208) c3(arf-GAP with GTPase, ANK repeat and PH domain-containing protein 1 isoform 1) c4(31085, 31086) c5(31087, 31088, 31089, 31090, 31091, 31092) c6( ) c7(fq, bN); #c1(AGAP1) c2(NP_001231817) c3(arf-GAP with GTPase, ANK repeat and PH domain-containing protein 1 isoform 3) c4(87557) c5(87558, 87559, 87560, 87561, 87562, 87563) c6( ) c7(fq, bN); #c1(AGAP1) c2(NP_055729) c3(arf-GAP with GTPase, ANK repeat and PH domain-containing protein 1 isoform 2) c4(183152) c5(183153, 183154, 183155, 183156, 183157, 183158) c6( ) c7(fq, bN); #c1(AGAP1) c2(XP_011508849) c3(arf-GAP with GTPase, ANK repeat and PH domain-containing protein 1 isoform X3) c4( ) c5(299437, 299438, 299439, 299440, 299441, 299442) c6( ) c7(fq, bN); #c1(AGAP1) c2(XP_011508851) c3(arf-GAP with GTPase, ANK repeat and PH domain-containing protein 1 isoform X5) c4(299443) c5(299444, 299445, 299446, 299447, 299448, 299449) c6( ) c7(fq, bN); #c1(AGAP2) c2(NP_001116244) c3(arf-GAP with GTPase, ANK repeat and PH domain-containing protein 2 isoform PIKE-L) c4(43711) c5(43712, 43713, 43714, 43715, 43716, 43717) c6( ) c7(T, d, Ew, Ls, ae, ad); #c1(AGAP2) c2(NP_055585) c3(arf-GAP with GTPase, ANK repeat and PH domain-containing protein 2 isoform PIKE-A) c4(182115) c5(182116, 182117, 182118, 182119, 182120, 182121) c6( ) c7(T, d, Ew, Ls, ae, ad); #c1(AGAP2) c2(XP_005268682) c3(arf-GAP with GTPase, ANK repeat and PH domain-containing protein 2 isoform X1) c4( ) c5(282053) c6( ) c7(T, d, Ew, Ls, ae, ad); #c1(AGAP3) c2(NP_001036000) c3(arf-GAP with GTPase, ANK repeat and PH domain-containing protein 3 isoform b) c4(33649) c5(33650, 33651, 33652, 33653, 33654, 33655) c6( ) c7(fq); #c1(AGAP3) c2(NP_001268229) c3(arf-GAP with GTPase, ANK repeat and PH domain-containing protein 3 isoform c) c4(99705) c5(99706, 99707, 99708, 99709, 99710, 99711, 99712, 99713, 99714, 99715, 99716) c6( ) c7(fq); #c1(AGAP3) c2(NP_114152) c3(arf-GAP with GTPase, ANK repeat and PH domain-containing protein 3 isoform a) c4(222367, 222368, 222369) c5(222370, 222371, 222372, 222373, 222374, 222375, 222376, 222377, 222378, 222379, 222380, 222381, 222382) c6( ) c7(fq); #c1(AGAP3) c2(XP_005250000) c3(arf-GAP with GTPase, ANK repeat and PH domain-containing protein 3 isoform X3) c4( ) c5(269991, 269992, 269993) c6( ) c7(fq); #c1(AGAP4) c2(NP_597703) c3(arf-GAP with GTPase, ANK repeat and PH domain-containing protein 4 isoform 2) c4(235058, 235059, 235060) c5(235061, 235062, 235063, 235064, 235065, 235066, 235067, 235068, 235069, 235070, 235071, 235072, 235073, 235074, 235075, 235076, 235077, 235078) c6( ) c7(T, B); #c1(AGAP4) c2(XP_005271857) c3(arf-GAP with GTPase, ANK repeat and PH domain-containing protein 4 isoform X2) c4(283610, 283611) c5(283612, 283613) c6( ) c7(T, B); #c1(AGAP4) c2(XP_011537571) c3(arf-GAP with GTPase, ANK repeat and PH domain-containing protein 4 isoform X1) c4( ) c5(335154, 335155, 335156, 335157, 335158) c6( ) c7(T, B); #c1(AGBL1) c2(NP_689549) c3(cytosolic carboxypeptidase 4) c4(243083, 243084) c5(243085, 243086, 243087, 243088, 243089, 243090, 243091, 243092, 243093, 243094, 243095, 243096) c6( ) c7(hb, vo, Lu, gv, Lt); #c1(AGBL1) c2(XP_011519528) c3(cytosolic carboxypeptidase 4 isoform X1) c4(314178) c50) c6( ) c7(hb, vo, Lu, gv, Lt); #c1(AGBL1) c2(XP_011519529) c3(cytosolic carboxypeptidase 4 isoform X2) c4( ) c5(314179, 314180, 314181, 314182, 314183, 314184) c6( ) c7(hb, vo, Lu, gv, Lt); #c1(AGBL2) c2(NP_079059) c3(cytosolic carboxypeptidase 2) c4(216823, 216824) c5(216825, 216826, 216827, 216828, 216829, 216830, 216831, 216832, 216833, 216834, 216835, 216836, 216837, 216838, 216839, 216840) c6( ) c7(nv, y, ai, CQ); #c1(AGBL2) c2(XP_006718390) c3(cytosolic carboxypeptidase 2 isoform X4) c4(291816) c5(291817) c6( ) c7(nv, y, ai, CQ); #c1(AGBL2) c2(XP_006718391) c3(cytosolic carboxypeptidase 2 isoform X8) c4( ) c5(291818, 291819, 291820, 291821, 291822) c6( ) c7(nv, y, ai, CQ); #c1(AGBL3) c2(NP_848658) c3(cytosolic carboxypeptidase 3) c4(254096, 254097, 254098) c5(254099, 254100, 254101, 254102, 254103, 254104, 254105, 254106, 254107, 254108, 254109, 254/10) c6( ) c7(Hr); #c1(AGBL3) c2(XP_011514424) c3(cytosolic carboxypeptidase 3 isoform X3) c4( ) c5(307127, 307128, 307129, 307130, 307131, 307132) c6( ) c7(Hr); #c1(AGBL4) c2(XP_011540611) c3(cytosolic carboxypeptidase 6 isoform X2) c4(339108) c5( ) c6( ) c7(IB, W); #c1(AGBL4) c2(XP_011540613) c3(cytosolic carboxypeptidase 6 isoform X5) c4( ) c5(339109, 339110, 339111, 339112, 339113, 339114) c6( ) c7(IB, W); #c1(AGER) c2(NP_001127) c3(advanced glycosylation end product-specific receptor isoform 1 precursor) c4( ) c5( ) c6( ) c7(bM, T, aC, ea, e, ci, Lw, bv, dY, Bc, M, K, H, aa, aW, vs, g, y, eM, vy, Hb, R, m, vF, dD, Ly, Hm, aw, fN, BH, cQ, aj, Lv, eV, A, kT, mg, at, uz, Dp, B, bL, eR, fG, Lx, eX, cz, cR, fF, wo, v, j, sl, V, J, uT, ns, aT, cy, bq, ae, af, eu, bA, ak, bf, iU, Y, r, en, bu, wA, rY, bN, rR, ac, mf, dy, fD, ao, kh, wi, dG, eq, ad, av, sN, LA, sj, uQ, uX, lR, cW, ff, cr, eZ, aF, iV, bz, LB, Bv, aS, Lz, lC, X, us); #c1(AGER) c2(NP_001193858) c3(advanced glycosylation end product-specific receptor isoform 2 precursor) c4(82771, 82772, 82773, 82774) c5( ) c6( ) c7(bM, T, aC, ea, e, ci, Lw, bv, dY, Bc, M, K, H, aa, aW, vs, g, y, eM, vy, Hb, R, m, vF, dD, Ly, Hm, aw, fN, BH, cQ, aj, Lv, eV, A, kT, mg, at, uz, Dp, B, bL, eR, fG, Lx, eX, cz, cR, fF, wo, v, j, sl, V, J, uT, ns, aT, cy, bq, ae, af, eu, bA, ak, bf, iU, Y, r, en, bu, wA, rY, bN, rR, ac, mf, dy, fD, ao, kh, wi, dG, eq, ad, av, sN, LA, sj, uQ, uX, lR, cW, ff, cr, eZ, aF, iV, bz, LB, Bv, aS, Lz, lC, X, us); #c1(AGER) c2(NP_001193861) c3(advanced glycosylation end product-specific receptor isoform 3 precursor) c4( ) c5(82775, 82776, 82777, 82778, 82779, 82780, 82781, 82782, 82783, 82784, 82785, 82786, 82787, 82788, 82789, 82790, 82791, 82792, 82793, 82794, 82795, 82796, 82797, 82798, 82799, 82800, 82801, 82802, 82803, 82804, 82805, 82806, 82807, 82808, 82809, 82810, 82811, 82812, 82813, 82814, 82815, 82816, 82817, 82818, 82819, 82820, 82821, 82822) c6( ) c7(bM, T, aC, ea, e, ci, Lw, bv, dY, Bc, M, K, H, aa, aW, vs, g, y, eM, vy, Hb, R, m, vF, dD, Ly, Hm, aw, fN, BH, cQ, aj, Lv, eV, A, kT, mg, at, uz, Dp, B, bL, eR, fG, Lx, eX, cz, cR, fF, wo, v, j, sl, V, J, uT, ns, aT, cy, bq, ae, af, eu, bA, ak, bf, iU, Y, r, en, bu, wA, rY, bN, rR, ac, mf, dy, fD, ao, kh, wi, dG, eq, ad, av, sN, LA, sj, ul, uX, lR, cW, ff, cr, eZ, aF, iV, bz, LB, Bv, aS, Lz, lC, X, us); #c1(AGER) c2(NP_001193863) c3(advanced glycosylation end product-specific receptor isoform 4 precursor) c4(82823, 82824, 82825, 82826, 82827, 82828) c5(82829, 82830, 82831, 82832, 82833, 82834, 82835, 82836, 82837, 82838, 82839, 82840, 82841, 82842, 82843, 82844, 82845, 82846, 82847, 82848, 82849, 82850, 82851, 82852, 82853, 82854, 82855, 82856, 82857, 82858, 82859, 82860, 82861, 82862, 82863, 82864, 82865, 82866, 82867, 82868, 82869, 82870, 82871, 82872, 82873, 82874, 82875, 82876, 82877, 82878, 82879, 82880, 82881, 82882, 82883, 82884) c6( ) c7(bM, T, aC, ea, e, ci, Lw, bv, dY, Bc, M, K, H, aa, aW, vs, g, y, eM, vy, Hb, R, m, vF, dD, Ly, Hm, aw, fN, BH, c, aj, Lv, eV, A, kT, mg, at, uz, Dp, B, bL, eR, fG, Lx, eX, cz, CR, fF, wo, v, j, sl, V, J, uT, ns, aT, cy, bq, ae, af, eu, bA, ak, bf, iU, Y, r, en, bu, wA, rY, bN, rR, ac, mf, dy, fD, ao, kh, wi, dG, eq, ad, av, sN, LA, sj, uQ, uX, lR, cW, ff, cr, eZ, aF, iV, bz, LB, Bv, aS, Lz, lC, X, us); #c1(AGER) c2(NP_001193883) c3(advanced glycosylation end product-specific receptor isoform & precursor) c4( ) c5(82910, 82911, 82912, 82913, 82914, 82915, 82916, 82917, 82918, 82919, 82920, 82921, 82922, 82923, 82924, 82925, 82926, 82927, 82928, 82929, 82930, 82931, 82932, 82933, 82934, 82935, 82936, 82937, 82938, 82939, 82940, 82941, 82942, 82943, 82944, 82945, 82946, 82947, 82948, 82949, 82950, 82951, 82952, 82953, 82954, 82955, 82956, 82957, 82958, 82959, 82960, 82961, 82962, 82963, 82964, 82965, 82966, 82967, 82968, 82969, 82970, 82971, 82972, 82973, 82974, 82975, 82976, 82977, 82978, 82979, 82980, 82981, 82982, 82983, 82984, 82985, 82986, 82987, 82988, 82989, 82990, 82991, 82992, 82993, 82994, 82995, 82996, 82997, 82998, 82999, 83000, 83001, 83002, 83003, 83004, 83005, 83006, 83007, 83008, 83009, 83010, 83011, 83012, 83013, 83014, 83015, 83016, 83017, 83018, 83019, 83020, 83021) c6( ) c7(bM, T, aC, ea, e, ci, Lw, bv, dY, Bc, M, K, H, aa, aW, vs, g, y, eM, vy, Hb, R, m, vF, dD, Ly, Hm, aw, fN, BH, cQ, aj, Lv, eV, A, kT, mg, at, uz, Dp, B, bL, eR, fG, Lx, eX, cz, cR, fF, wo, v, j, sl, V, J, uT, ns, aT, cy, bq, ae, af, eu, bA, ak, bf, iU, Y, r, en, bu, wA, rY, bN, rR, ac, mf, dy, fD, ao, kh, wi, dG, eq, ad, av, sN, LA, sj, uQ, uX, lR, cW, ff, cr, eZ, aF, iV, bz, LB, Bv, aS, Lz, lC, X, us); #c1(AGER) c2(NP_001193895) c3(advanced glycosylation end product-specific receptor isoform 6 precursor) c4(83069, 83070, 83071) c5(83072, 83073, 83074, 83075, 83076, 83077, 83078, 83079, 83080, 83081, 83082, 83083, 83084, 83085, 83086, 83087, 83088, 83089, 83090, 83091, 83092, 83093, 83094, 83095, 83096, 83097, 83098, 83099) c6( ) c7(bM, T, aC, ea, e, ci, Lw, bv, dY, Bc, M, K, H, aa, aW, vs, g, y, eM, vy, Hb, R, m, vF, dD, Ly, Hm, aw, fN, BH, cQ, aj, Lv, eV, A, kT, mg, at, uz, Dp, B, bL, eR, fG, Lx, eX, cz, CR, fF, wo, v, j, sl, V, J, uT, ns, aT, cy, bq, ae, af, eu, bA, ak, bf, iU, Y, r, en, bu, wA, rY, bN, rR, ac, mf, dy, fD, ao, kh, wi, dG, eq, ad, av, sN, LA, sj, uQ, uX, lR, cW, ff, cr, eZ, aF, iV, bz, LB, Bv, aS, Lz, lC, X, us); #c1(AGER) c2(NP_751947) c3(advanced glycosylation end product-specific receptor isoform 7 precursor) c4(248573, 248574) c5(248575, 248576, 248577, 248578, 248579, 248580, 248581, 248582, 248583, 248584, 248585, 248586, 248587, 248588, 248589, 248590, 248591, 248592, 248593, 248594, 248595, 248596, 248597, 248598, 248599, 248600, 248601, 248602) c6( ) c7(bM, T, aC, ea, e, ci, Lw, bv, dY, Bc, M, K, H, aa, aW, vs, g, y, eM, vy, Hb, R, m, vF, dD, Ly, Hm, aw, fN, BH, cQ, aj, Lv, eV, A, kT, mg, at, uz, Dp, B, bL, eR, fG, Lx, eX, cz, cR, fF, wo, v, j, sl, V, J, uT, ns, aT, cy, bq, ae, af, eu, bA, ak, bf, iU, Y, r, en, bu, wA, rY, bN, rR, ac, mf, dy, fD, ao, kh, wi, dG, eq, ad, av, sN, LA, sj, uQ, uX, lR, cW, ff, cr, eZ, aF, iV, bz, LB, Bv, aS, Lz, lC, X, us); #c1(AGFG1) c2(NP_001128659) c3(arf-GAP domain and FG repeat-containing protein 1 isoform 1) c4(50461, 50462) c5(50463, 50464, 50465, 50466, 50467, 50468, 50469, 50470, 50471, 50472, 50473, 50474) c6( ) c7(AT, T, dA, eM, aW, nx, yG, bo, aT, aj, dL, k, nA, EA, y, eL, J, oU); #c1(AGFG1) c2(NP_001128660) c3(arf-GAP domain and FG repeat-containing protein 1 isoform 3) c4(50475) c5(50476, 50477, 50478, 50479, 50480, 50481) c6( ) c7(AT, T, dA, eM, aW, nx, yG, bo, aT, aj, dL, k, nA, EA, y, eL, J, oU); #c1(AGFG1) c2(NP_001128661) c3(arf-GAP domain and FG repeat-containing protein 1 isoform 4) c4(50482) c5(50483, 50484, 50485, 50486, 50487, 50488) c6( ) c7(AT, T, dA, eM, aW, nx, yG, bo, aT, aj, dL, k, nA, EA, y, eL, J, oU); #c1(AGFG1) c2(NP_004495) c3(arf-GAP domain and FG repeat-containing protein 1 isoform 2) c4(146929) c5(146930, 146931, 146932, 146933, 146934, 146935) c6( ) c7(AT, T, dA, eM, aW, nx, yG, bo, aT, aj, dL, k, nA, EA, y, eL, J, oU); #c1(AGFG2) c2(NP_006067) c3(arf-GAP domain and FG repeat-containing protein 2) c4(161629) c5(161630, 161631, 161632, 161633, 161634, 161635, 161636, 161637, 161638, 161639, 161640, 161641) c6( ) c7(ck, T, iT, iU, h, bw, m, hf, hF, bt, br); #c1(AGFG2) c2(XP_005250363) c3(arf-GAP domain and FG repeat-containing protein 2 isoform X1) c4(270267) c5( ) c6( ) c7(ck, T, iT, iU, h, bw, m, hf, hF, bt, br); #c1(AGGF1) c2(NP_060516) c3(angiogenic factor with G patch and FHA domains 1) c4(196321, 196322) c5(196323, 196324, 196325, 196326, 196327, 196328, 196329, 196330, 196331, 196332, 196333, 196334) c6( ) c7(bE, LC, bB, aS, LD, Cd, Cb); #c1(AGK) c2(XP_005250080) c3(acylglycerol kinase, mitochondrial isoform X2) c4( ) c5(270085, 270086, 270087, 270088, 270089, 270090, 270091, 270092, 270093, 270094, 270095, 270096, 270097, 270098, 270099, 270100) c6( ) c7(ae, T, cc, aW, aT, it, h, LE, LF, og, ad); #c1(AGK) c2(XP_011514699) c3(acylglycerol kinase, mitochondrial isoform X1) c4(307339, 307340, 307341) c5(307342, 307343, 307344, 307345, 307346, 307347) c6( ) c7(ae, T, cc, aW, aT, it, h, LE, LF, og, ad); #c1(AGM0) c2(NP_001004320) c3 (alkylglycerol monooxygenase) c4(20584) c5(20585, 20586, 20587, 20588, 20589, 20590) c6( ) c7(bL, us); #c1(AG02) c2(NP_001158095) c3(protein argonaute-2 isoform 2) c4(63134) c5(63135, 63136, 63137, 63138, 63139, 63140) c6( ) c7(gh, T, e, jx, aA, fd, fr, ci, zU, LI, ad, LH, hX, aE, ec, aT, vw, hP, K, LG, ae, eL, aW, Y, r, aq, cu, LJ, t, bz, es, cA, ig, iT, zG, m, bt, B); #c1(AG02) c2(NP_036286) c3(protein argonaute-2 isoform 1) c4(172840) c5(172841, 172842, 172843, 172844, 172845, 172846) c6( ) c7(gh, T, e, jx, aA, fd, fr, ci, zU, LI, ad, LH, hX, aE, ec, aT, vw, hP, K, LG, ae, eL, aW, Y, r, aq, cu, LJ, t, bz, es, cA, ig, iT, zG, m, bt, B); #c1(AGPAT1) c2(XP_005248862) c3(1-acyl-sn-glycerol-3-phosphate acyltransferase alpha isoform X1) c4(268672, 268673, 268674) c5(268675, 268676, 268677, 268678, 268679, 268680, 268681, 268682, 268683, 268684, 268685, 268686, 268687, 268688, 268689, 268690, 268691, 268692, 268693, 268694, 268695, 268696, 268697, 268698) c6( ) c7(bL, T, A, aE, W, fc, cr, eZ, cP, GR); #c1(AGPAT1) c2(XP_011512536) c3(1-acyl-sn-glycerol-3-phosphate acyltransferase alpha isoform X2) c4( ) c5(303200, 303201, 303202, 303203, 303204, 303205, 303206, 303207, 303208, 303209, 303210, 303211, 303212, 303213, 303214, 303215, 303216, 303217, 303218, 303219, 303220, 303221, 303222, 303223, 303224, 303225, 303226, 303227, 303228, 303229, 303230, 303231, 303232, 303233, 303234, 303235, 303236, 303237, 303238, 303239, 303240, 303241, 303242, 303243, 303244, 303245, 303246, 303247, 303248, 303249, 303250, 303251, 303252, 303253, 303254, 303255, 303256, 303257, 303258, 303259, 303260, 303261, 303262, 303263, 303264, 303265, 303266, 303267, 303268, 303269, 303270, 303271, 303272, 303273, 303274, 303275, 303276, 303277, 303278, 303279, 303280, 303281, 303282, 303283, 303284, 303285, 303286, 303287, 303288, 303289, 303290, 303291, 303292, 303293, 303294, 303295, 303296, 303297, 303298, 303299, 303300, 303301, 303302, 303303, 303304, 303305, 303306, 303307, 303308, 303309, 303310, 303311, 303312, 303313, 303314, 303315, 303316, 303317, 303318, 303319, 303320, 303321, 303322, 303323, 303324, 303325, 303326, 303327, 303328, 303329, 303330, 303331, 303332, 303333, 303334, 303335, 303336, 303337, 303338, 303339, 303340, 303341, 303342, 303343, 303344, 303345, 303346, 303347, 303348, 303349, 303350, 303351, 303352, 303353, 303354, 303355, 303356, 303357, 303358, 303359, 303360, 303361, 303362, 303363, 303364, 303365, 303366, 303367, 303368, 303369, 303370, 303371, 303372, 303373, 303374, 303375) c6( ) c7(bL, T, A, aE, W, fc, cr, eZ, cP, GR); #c1(AGPAT2) c2(NP_001012745) c3(1-acyl-sn-glycerol-3-phosphate acyltransferase beta isoform b precursor) c4(25253) c5(25254, 25255, 25256, 25257, 25258, 25259) c6( ) c7(T, aj, v, nA, eJ, J, LL, LN, vc, iU, LO, eH, fc, cl, eF, LK, t, GR, dr, JP, LM, hL, lZ, m, cP); #c1(AGPAT2) c2(NP_006403) c3(1-acyl-sn-glycerol-3-phosphate acyltransferase beta isoform a precursor) c4(164746) c5(164747, 164748, 164749, 164750, 164751, 164752, 164753, 164754, 164755, 164756) c6( ) c7(T, aj, v, nA, eJ, J, LL, LN, vc, iU, LO, eH, fc, cl, eF, LK, t, GR, dr, JP, LM, hL, lZ, m, cP); #c1(AGPAT3) c2(XP_006724093) c3(1-acyl-sn-glycerol-3-phosphate acyltransferase gamma isoform X1) c4(295705, 295706, 295707) c5(295708, 295709, 295710, 295711, 295712, 295713, 295714, 295715, 295716, 295717, 295718, 295719, 295720, 295721, 295722, 295723, 295724, 295725, 295726, 295727, 295728, 295729, 295730, 295731, 295732, 295733, 295734, 295735, 295736, 295737, 295738, 295739, 295740, 295741, 295742, 295743, 295744, 295745, 295746, 295747, 295748, 295749, 295750) c6( ) c7(U); #c1(AGPAT3) c2(XP_006724094) c3(1-acyl-sn-glycerol-3-phosphate acyltransferase gamma isoform X7) c4( ) c5(295751) c6( ) c7(U); #c1(AGPAT3) c2(XP_011527963) c3(1-acyl-sn-glycerol-3-phosphate acyltransferase gamma isoform X3) c4( ) c5(323020, 323021, 323022) c6( ) c7(U); #c1(AGPAT3) c2(XP_011527964) c3(1-acyl-sn-glycerol-3-phosphate acyltransferase gamma isoform X4) c4(323023, 323024, 323025) c5(323026, 323027, 323028, 323029, 323030, 323031, 323032, 323033, 323034, 323035, 323036, 323037, 323038) c6( ) c7(U); #c1(AGPAT3) c2(XP_011527965) c3(1-acyl-sn-glycerol-3-phosphate acyltransferase gamma isoform X5) c4( ) c5(323039, 323040) c6( ) c7(U); #c1(AGPAT3) c2(XP_011527966) c3(l-acyl-sn-glycerol-3-phosphate acyltransferase gamma isoform X6) c4( ) c5(323041) c6( ) c7(U); #c1(AGPAT4) c2(NP_064518) c3(1-acyl-sn-glycerol-3-phosphate acyltransferase delta) c4(202838, 202839, 202840, 202841) c5(202842, 202843, 202844, 202845, 202846, 202847, 202848) c6( ) c7(pN); #c1(AGPAT4) c2(XP_005267111) c3(1-acyl-sn-glycerol-3-phosphate acyltransferase delta isoform X3) c4(280755, 280756) c5(280757, 280758, 280759, 280760, 280761, 280762, 280763, 280764, 280765, 280766, 280767, 280768) c6( ) c7(pN); #c1(AGPAT4) c2(XP_006715577) c3(1-acyl-sn-glycerol-3-phosphate acyltransferase delta isoform X2) c4(289616) c5( ) c6( ) c7(pN); #c1(AGPAT4) c2(XP_006715578) c3(1-acyl-sn-glycerol-3-phosphate acyltransferase delta isoform X4) c4( ) c5(289617, 289618, 289619, 289620, 289621, 289622, 289623, 289624, 289625, 289626, 289627, 289628, 289629, 289630, 289631, 289632, 289633, 289634, 289635, 289636, 289637, 289638, 289639) c6( ) c7(pN); #c1(AGPAT9) c2(NP_001243350) c3(glycerol-3-phosphate acyltransferase 3) c4(90471) c5(90472, 90473, 90474, 90475, 90476, 90477, 90478, 90479, 90480, 90481) c6( ) c7(aW, yx, aw, al, v, j, K); #c1(AGPS) c2(NP_003650) c3(alkyldihydroxyacetonephosphate synthase, peroxisomal precursor) c4(139932, 139933) c5(139934, 139935, 139936, 139937, 139938, 139939) c6( ) c7(LA, jp, sO, LP, ey, LR, af); #c1(AGPS) c2(XP_011510344) c3(alkyldihydroxyacetonephosphate synthase, peroxisomal isoform X1) c4( ) c5(300893, 300894, 300895, 300896, 300897, 300898) c6( ) c7(LQ, jp, sO, LP, ey, LR, af); #c1(AGR2) c2(NP_006399) c3 (anterior gradient protein 2 homolog precursor) c4(164718, 164719, 164720) c5(164721, 164722, 164723, 164724, 164725, 164726, 164727, 164728, 164729, 164730, 164731, 164732, 164733, 164734, 164735, 164736, 164737, 164738) c6( ) c7(bM, T, aC, LX, n, Fb, j, ci, q, ad, dP, Bl, aE, LY, LW, aT, K, ae, s, aW, LZ, r, fe, kp, LU, g, iV, LV, t, LS, DF, ar, gX, m, cE, B, LT); #c1(AGR3) c2(NP_789783) c3(anterior gradient protein 3 homolog precursor) c4(252287) c5(252288, 252289, 252290, 252291, 252292, 252293) c6( ) c7(Mb, Ma, aT, fe, ci, m, t, ae, B, ad); #c1(AGRN) c2(XP_011539733) c3(agrin isoform X5) c4( ) c5(337955) c6( ) c7(xA, Mc, Md, bt, fq, af, bN); #c1(AGRP) c2(XP_011521229) c3 (agouti-related protein isoform X1) c4(316146) c5(316147, 316148, 316149, 316150, 316151, 316152) c6( ) c7(bL, T, gU, jx, Me, ml, cz, aj, gf, v, rc, J, Mg, iX, dV, h, qM, rz, d, oV, LO, gC, Mf, Lk, LI, GR, bJ, xK, dc, mf, ei, eg, bt); #c1(AGT) c2(NP_000020) c3(angiotensinogen preproprotein) c4(104, 105) c5(106, 107, 108, 109, 110, 111, 112, 113, 114, 115, 116, 117) c6( ) c7(Mh, bM, T, Fj, tQ, wH, ea, if, e, al, Av, eb, MF, ur, ci, aY, GK, wM, dY, aE, ws, il, Bc, M, dV, Mx, iv, K, qM, wt, dQ, eL, bc, eN, hm, tw, zQ, Mq, HI, aW, Fe, az, hl, fc, vt, vs, pD, zC, aB, g, ve, uC, cm, mZ, fT, wE, mx, vy, wB, uo, Hb, m, lS, fy, MI, dD, uH, Ly, vl, wJ, My, aw, cQ, aj, Mi, ct, Ms, qN, fr, zP, ww, eh, cd, JW, Mo, cc, no, Mm, do, Mv, ql, ec, we, wG, zf, sW, jy, dl, ey, wo, bl, mV, mY, mU, A, MD, fl, aq, wd, kp, Hu, fe, dw, ty, cM, sf, mX, hq, kT, W, dC, vb, FV, wk, bm, bo, tl, uz, at, va, gv, eo, gg, bD, mT, B, um, vQ, bL, jv, tf, eR, mr, wL, dk, oe, uv, fG, yk, aA, cR, tc, sM, mS, j, cz, C, sl, fF, uW, V, v, Mk, J, q, vp, il, Mp, ga, uT, hb, nX, qp, rS, aT, yu, cy, bK, bq, sR, Fg, ae, af, eu, bA, DX, kl, su, Mr, s, bf, Y, r, iy, wl, uU, en, cl, wA, Mu, t, pb, sk, wg, ac, c, pZ, jx, hc, MB, dh, fP, hO, cS, Mn, dy, ud, Mt, Mj, vi, vq, sY, wi, qb, aZ, dM, uG, vr, OR, vG, bS, uu, jE, dG, ah, MA, eq, tK, MC, ad, dZ, kL, dP, fU, hX, F, sN, mW, oz, vJ, lC, ts, oW, vw, vc, uD, nJ, vP, vl, sj, mu, ul, ux, ME, d, lR, tb, cH, cW, ff, wc, gC, eZ, Mz, dq, yy, aF, si, Z, iR, bz, qa, ek, LB, D, gk, pE, ag, gZ, Mw, tr, cP, aS, bC, hE, tP, be, ei, us); #c1(AGTPBP1) c2(NP_001273644) c3 (cytosolic carboxypeptidase 1 isoform a) c4( ) c5(105527, 105528, 105529, 105530, 105531, 105532, 105533) c6( ) c7(bM, HC, n, CQ, cm, nC, fq, MG); #c1(AGTPBP1) c2(NP_001273646) c3(cytosolic carboxypeptidase 1 isoform c) c4(105534, 105535) c5(105536, 105537, 105538, 105539, 105540, 105541, 105542) c6( ) c7(bM, HC, n, CQ, cm, nC, fq, MG); #c1(AGTPBP1) c2(NP_056054) c3(cytosolic carboxypeptidase 1 isoform b) c4(185405) c5(185406, 185407, 185408, 185409, 185410, 185411) c6( ) c7(bM, HC, n, CQ, cm, nC, fq, MG); #c1(AGTPBP1) c2(XP_005251905) c3(cytosolic carboxypeptidase 1 isoform X1) c4(271276) c5(271277, 271278, 271279, 271280, 271281, 271282) c6( ) c7(bM, HC, n, CQ, cm, nC, fq, MG); #c1(AGTPBP1) c2(XP_011516721) c3(cytosolic carboxypeptidase 1 isoform X4) c4(310179) c5( ) c6( ) c7(bM, HC, n, CQ, cm, nC, fq, MG); #c1(AGTPBP1) c2(XP_011516723) c3(cytosolic carboxypeptidase 1 isoform X6) c4(310180) c5(310181, 310182, 310183, 310184, 310185, 310186, 310187, 310188, 310189, 310190, 310191, 310192, 310193, 310194) c6( ) c7(bM, HC, n, CQ, cm, nC, fq, MG); #c1(AGTR1) c2(NP_004826) c3(type-l angiotensin 11 receptor isoform 2) c4( ) c5(150101, 150102, 150103) c6( ) c7(ID, bM, T, Fj, ea, e, al, eb, ur, ci, Lw, aY, dY, aE, il, Bc, M, iv, K, wt, dQ, MO, eL, eN, hm, tw, ly, aW, fc, Au, pD, aB, g, uC, wT, AM, wE, mx, vy, wB, uo, m, sr, dD, vl, sQ, wJ, aw, cQ, aj, fr, ct, uE, zP, JW, cd, Bl, b, cc, ec, wG, ql, jy, dL, ey, A, MQ, aq, wd, kp, Hu, dw, ty, CM, ML, lx, sf, KT, W, vb, bo, lf, hL, MM, hR, uz, at, bm, gg, bD, sZ, B, um, bL, tf, eR, mr, wL, oe, uv, aA, cR, tc, cz, fF, j, v, MN, wo, AO, J, MK, ga, uT, nX, rS, MP, tF, MI, aT, cy, sR, Fg, ae, af, eu, su, tA, s, bf, Y, r, wl, sV, uU, cl, Mu, t, f, pb, wg, ac, c, ig, hc, fP, cS, dy, sY, MH, aZ, dM, aD, uG, vr, OR, vG, bS, uu, dG, ah, eq, tK, ad, tX, fU, dP, F, oz, vJ, vw, vc, vB, tt, uD, vP, sj, mu, uQ, uX, lR, qZ, cH, cW, eZ, dq, aF, Z, iR, qa, eK, LB, D, gk, ag, uR, gZ, nT, MJ, aS, tr, ei, ME, i, lC, vt, BA); #c1(AGTR1) c2(NP_033611) c3(type-1 angiotensin 11 receptor isoform 1) c4( ) c5(172267, 172268, 172269, 172270, 172271, 172272, 172273, 172274, 172275, 172276, 172277, 172278, 172279, 172280, 172281, 172282, 172283, 172284, 172285, 172286, 172287, 172288, 172289, 172290, 172291, 172292, 172293, 172294, 172295) c6( ) c7(ID, bM, T, Fj, ea, e, al, eb, ur, ci, Lw, aY, dY, aE, il, Bc, M, iv, K, wt, dQ, MO, eL, eN, hm, tw, ly, aW, fc, Au, pD, aB, g, uC, wT, AM, wE, mx, vy, wB, uo, m, sr, dD, vl, sQ, wJ, aw, cQ, aj, fr, ct, uE, zP, JW, cd, Bl, b, cc, ec, wG, ql, jy, dl, ey, A, MQ, aq, wd, kp, Hu, dw, ty, cM, ML, lx, sf, kT, W, vb, bo, lf, hL, MM, hR, uz, at, bm, gg, bD, sZ, B, um, bL, tf, eR, mr, wL, oe, uv, aA, cR, tc, cz, fF, j, v, MN, wo, AD, J, MK, ga, uT, nX, rS, MP, tF, MI, aT, cy, sR, Fg, ae, af, eu, su, tA, s, bf, Y, r, wl, sV, uU, cl, Mu, t, f, pb, wg, ac, c, ig, hc, fP, cS, dy, sY, MH, aZ, dM, aD, uG, vr, oR, vG, bS, uu, dG, ah, eq, tK, ad, tx, fU, dP, F, oz, vJ, vw, vc, vB, tt, uD, vP, sj, mu, uQ, uX, lR, qZ, cH, cW, eZ, dq, aF, Z, iR, qa, ek, LB, D, gk, ag, uR, gZ, nT, MJ, aS, tr, ei, ME, i, lC, vt, BA); #c1(AGTR1) c2(NP_114438) c3(type-1 angiotensin 11 receptor isoform 3) c4(222795) c5(222796, 222797, 222798, 222799, 222800, 222801) c6( ) c7(ID, bM, T, Fj, ea, e, al, eb, ur, ci, Lw, aY, dY, aE, il, Bc, M, iv, K, wt, dQ, MO, eL, eN, hm, tw, ly, aW, fc, Au, pD, aB, g, uC, wT, AM, wE, mx, vy, wB, uo, m, sr, dD, vl, sQ, wJ, aw, cQ, aj, fr, ct, uE, zP, JW, cd, BL, b, cc, ec, wG, ql, jy, dL, ey, A, MQ, aq, wd, kp, Hu, dw, ty, cM, ML, lx, sf, KT, W, vb, bo, lf, hL, MM, hR, uz, at, bm, gg, bD, sZ, B, um, bl, tf, eR, mr, wl, oe, uv, aA, cR, tc, cz, fF, j, v, MN, wo, AO, J, MK, ga, uT, nX, rS, MP, tF, MI, aT, cy, sR, Fg, ae, af, eu, su, tA, s, bf, Y, r, wl, sV, ul, cl, Mu, t, f, pb, wg, ac, c, ig, hc, fP, cS, dy, sY, MH, aZ, dM, aD, uG, vr, OR, vG, bS, uu, dG, ah, eq, tK, ad, tX, fU, dP, F, oz, vJ, vw, vc, vB, tt, uD, vP, sj, mu, uQ, uX, lR, qZ, cH, cW, eZ, dq, aF, Z, iR, qa, ek, LB, D, gk, ag, uR, gZ, nT, MJ, aS, tr, ei, ME, i, lC, vt, BA); #c1(AGTR2) c2(XP_011535835) c3(type-2 angiotensin 1 receptor isoform X1) c4(333040) c5(333041, 333042, 333043, 333044, 333045, 333046) c6( ) c7(bM, T, ea, e, ci, il, K, dQ, aW, zE, uC, vA, mx, vy, fy, in, dD, aw, aj, nA, zP, MT, cd, wG, h, lG, ey, uV, A, sm, Hu, dw, zZ, op, hq, bo, gg, B, vQ, bL, eR, oe, aA, cz, v, j, C, sl, il, MR, tl, aT, cy, jj, bq, ae, af, kl, s, Mu, ac, ig, hc, dy, ao, HQ, MH, aZ, uO, dG, eq, ad, dP, fU, av, fX, MS, vw, jo, vP, sj, uQ, uX, nf, Z, Hv, qa, LB, D, ei, kS); #c1(AGXT) c2(NP_000021) c3(serine-pyruvate aminotransferase) c4(118) c5(119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210) c6( ) c7(Mh, bL, T, bM, eR, wH, wJ, aZ, yk, e, if, aw, cz, ff, eb, cQ, aj, j, ci, ur, uu, ah, aY, tK, v, vm, aR, J, hX, qN, no, hW, vJ, il, M, aT, MU, K, Cy, hm, kl, d, Mq, xU, lR, cH, cW, aW, MD, MX, fe, dq, uU, g, MV, fr, bz, dr, qa, hq, mO, Dy, ac, dC, z, MB, uz, MW, cS, aS, dy, ei, hE, tP, B); #c1(AGXT2) c2(NP_114/06) c3(alanine-glyoxylate aminotransferase 2, mitochondrial precursor) c4(222176) c5(222177, 222178, 222179, 222180, 222181, 222182) c6( ) c7(mO, cz); #c1(AHCY) c2(NP_000678) c3(adenosylhomocysteinase isoform 1) c4(14416, 14417) c5(14418, 14419, 14420, 14421, 14422, 14423, 14424) c6( ) c7(T, dD, ea, jx, cz, bB, fd, aj, eq, J, Na, MZ, Nc, DY, MY, Cr, eu, rL, bf, fO, hl, r, cm, ac, lp, bE, wE, Nb, bm, at, X); #c1(AHCY) c2(XP_011526962) c3(adenosylhomocysteinase isoform X1) c4( ) c5(321263, 321264, 321265, 321266, 321267) c6( ) c7(T, dD, ea, jx, cz, bB, fd, aj, eq, J, Na, MZ, Nc, DY, MY, Cr, eu, rL, bf, fO, hl, r, cm, ac, lp, bE, wE, Nb, bm, at, X); #c1(AHCYL2) c2(NP_001124192) c3(putative adenosylhomocysteinase 3 isoform b) c4(48489) c5(48490, 48491, 48492, 48493, 48494, 48495) c6( ) c7(cd, ci); #c1(AHCYL2) c2(NP_001124194) c3(putative adenosylhomocysteinase 3 isoform c) c4(48496) c5(48497, 48498, 48499, 48500, 48501, 48502) c6( ) c7(cd, ci); #c1(AHCYL2) c2(NP_001124195) c3(putative adenosylhomocysteinase 3 isoform d) c4(48503) c5(48504, 48505, 48506, 48507, 48508, 48509) c6( ) c7(cd, ci); #c1(AHCYL2) c2(NP_056143) c3 (putative adenosylhomocysteinase 3 isoform a) c4(185931) c5(185932, 185933, 185934, 185935, 185936, 185937) c6( ) c7(cd, ci); #c1(AHCYL2) c2(XP_011514288) c3(putative adenosylhomocysteinase 3 isoform X1) c4(306896) c5(306897, 306898, 306899, 306900, 306901, 306902) c6( ) c7(cd, ci); #c1(AHCYL2) c2(XP_011514290) c3(putative adenosylhomocysteinase 3 isoform X2) c4(306903, 306904) c5(306905, 306906, 306907, 306908, 306909, 306910, 306911, 306912, 306913, 306914, 306915, 306916) c6( ) c7(cd, ci); #c1(AHI1) c2(NP_001128304) c3(jouberin isoform b) c4(49815, 49816) c5(49817, 49818, 49819, 49820, 49821, 49822, 49823, 49824, 49825, 49826, 49827, 49828, 49829, 49830, 49831, 49832, 49833, 49834) c6( ) c7(bL, fM, dA, nx, jh, Hn, vm, v, nA, V, pd, Nh, hb, Ne, h, By, Nf, bK, K, Nj, rz, Nd, Ng, eG, je, lR, iU, Ni, gl, pR, nC, bN, ck, y, eU, iM, iT, Nk, FB, nw, dy, hE, xg, jr); #c1(AHI1) c2(XP_011534213) c3(jouberin isoform X1) c4(330548, 330549) c5(330550, 330551, 330552, 330553, 330554, 330555, 330556, 330557, 330558, 330559, 330560, 330561, 330562, 330563) c6( ) c7(bL, fM, dA, nx, jh, Hn, vm, v, nA, V, pd, Nh, hb, Ne, h, By, Nf, bK, K, Nj, rz, Nd, Ng, eG, je, lR, iU, Ni, gl, pR, nC, bN, ck, y, eU, iM, iT, Nk, FB, nw, dy, hE, xg, jr); #c1(AHI1) c2(XP_011534215) c3(jouberin isoform X2) c4( ) c5(330564, 330565, 330566, 330567, 330568) c6( ) c7(bL, fM, dA, nx, jh, Hn, vm, v, nA, V, pd, Nh, hb, Ne, h, By, Nf, bK, K, Nj, rz, Nd, Ng, eG, je, lR, iU, Ni, gl, pR, nC, bN, ck, y, eU, iM, iT, Nk, FB, nw, dy, hE, xg, jr); #c1(AHI1) c2(XP_011534217) c3(jouberin isoform X4) c4( ) c5(330569) c6( ) c7(bL, fM, dA, nx, jh, Hn, vm, v, nA, V, pd, Nh, hb, Ne, h, By, Nf, bK, K, Nj, rz, Nd, Ng, eG, je, lR, iU, Ni, gl, pR, nC, bN, ck, y, eU, iM, iT, Nk, FB, nw, dy, hE, xg, jr); #c1(AHI1) c2(XP_011534218) c3(jouberin isoform X5) c4(330570) c5( ) c6( ) c7(bL, fM, dA, nx, jh, Hn, vm, v, nA, V, pd, Nh, hb, Ne, h, By, Nf, bK, K, Nj, rz, Nd, Ng, eG, je, lR, iU, Ni, gl, pR, nC, bN, ck, y, eU, iM, iT, Nk, FB, nw, dy, hE, xg, jr); #c1(AHNAK) c2(NP_076965) c3(neuroblast differentiation-associated protein AHNAK isoform 2) c4(214496, 214497) c5(214498, 214499, 214500, 214501, 214502, 214503, 214504, 214505, 214506, 214507, 214508, 214509, 214510, 214511, 214512, 214513, 214514, 214515, 214516, 214517, 214518, 214519, 214520, 214521) c6( ) c7(bM, ib, NI, fr, K, Nm); #c1(AHNAK) c2(XP_005274298) c3(neuroblast differentiation-associated protein AHNAK isoform X1) c4(285040, 285041) c5(285042, 285043, 285044, 285045, 285046, 285047, 285048, 285049, 285050, 285051, 285052, 285053) c6( ) c7(bM, ib, NI, fr, K, Nm); #c1(AHNAK) c2(XP_005274300) c3(neuroblast differentiation-associated protein AHNAK isoform X4) c4(285054, 285055) c5( ) c6( ) c7(bM, ib, NI, fr, K, Nm); #c1(AHNAK) c2(XP_011543552) c3(neuroblast differentiation-associated protein AHNAK isoform X7) c4(342464) c5(342465, 342466, 342467, 342468, 342469, 342470) c6( ) c7(bM, ib, NI, fr, K, Nm); #c1(AHR) c2(NP_001612) c3(aryl hydrocarbon receptor precursor) c4(119193) c5(119194, 119195, 119196, 119197, 119198, 119199, 119200, 119201, 119202, 119203, 119204) c6( ) c7(bM, T, aC, ea, if, fd, bB, qj, ci, NF, x, xE, il, dV, K, aa, li, xQ, yo, aW, Nz, fc, Np, hl, g, eE, cm, y, eM, iA, nF, vA, NI, uJ, k, m, dD, gU, NA, zX, aw, n, Nv, ai, ft, Nn, No, Lv, dL, Nr, Nu, fe, aq, po, zZ, op, W, dB, hL, hR, at, bD, NB, it, B, oe, Nt, aA, cz, hy, Ns, bX, eA, jZ, Nw, aT, vH, NG, ae, af, jf, ND, NE, iU, Y, fO, r, cl, t, ik, ck, ov, iM, KP, dh, gX, Nx, fD, xb, LS, Gl, id, iW, uO, jh, dG, Jz, eq, ad, dP, av, py, iY, ji, lQ, nJ, uX, d, BN, je, cr, dN, Ej, aF, iV, iR, bz, Nq, es, NC, bE, Ny, pP, cP, ei, hE, iu, rk, pC); #c1(AHRR) c2(NP_001229341) c3(aryl hydrocarbon receptor repressor isoform 2) c4(84240) c5(84241, 84242, 84243, 84244, 84245, 84246) c6( ) c7(ck, dP, T, av, gU, ND, aW, NH, aT, vH, at, dL, Ns, bz); #c1(AHRR) c2(NP_065782) c3(aryl hydrocarbon receptor repressor isoform 1) c4(205413) c5(205414, 205415, 205416, 205417, 205418, 205419) c6( ) c7(ck, dP, T, av, gU, ND, aW, NH, aT, vH, at, dL, Ns, bz); #c1(AHSA1) c2(NP 036243) c3(activator of 90 kDa heat shock protein ATPase homolog 1) c4(172602, 172603) c5(172604, 172605, 172606, 172607, 172608, 172609, 172610, 172611, 172612, 172613, 172614, 172615) c6( ) c7(T, aC, ea, if, fd, bB, gf, lR, Lw, bv, xE, aE, rw, dV, K, aW, zC, g, as, eF, cm, bw, eM, cj, NO, Hb, m, NN, NK, dD, NJ, aj, BF, ct, qN, fr, cd, ai, Hx, jz, cU, h, fe, aq, NL, w, hR, B, bL, Dw, NO, fM, yk, lH, jx, aA, CR, cz, v, C, V, q, eA, eJ, yw, aT, kf, cy, ae, af, hM, wr, iU, Y, r, lo, en, t, iK, pb, ck, im, bt, ao, kh, jh, dG, eq, NI, iJ, ad, dP, av, CJ, F, MS, ds, ji, lQ, NM, NP, d, ff, eH, cr, af, bz, dr, bE, y, iu); #c1(AHSA2) c2(NP_689605) c3(activator of 90 kDa heat shock protein ATPase homolog 2) c4( ) c5(243237, 243238, 243239, 243240, 243241, 243242, 243243, 243244) c6( ) c7(hp); #c1(AHSA2) c2(XP_006712006) c3 (activator of 90 kDa heat shock protein ATPase homolog 2 isoform X3) c4(286336, 286337) c5(286338, 286339, 286340, 286341, 286342, 286343, 286344, 286345) c6( ) c7(hp); #c1(AHSG) c2(NP_001613) c3(alpha-2-HS-glycoprotein preproprotein) c4(119205, 119206) c5(119207, 119208, 119209, 119210, 119211, 119212, 119213, 119214, 119215, 119216, 119217, 119218) c6( ) c7(bL, T, dD, eR, fR, ea, e, aw, NT, ex, aj, v, j, eq, fo, q, J, dP, fU, NS, M, aT, dV, eV, K, af, bA, d, cW, aW, wc, ih, fc, r, aq, NR, rR, ac, U, mg, aS, dy, lS, ei, B, fl); #c1(AICDA) c2(NP_065712) c3(single-stranded DNA cytosine deaminase) c4(205034) c5(205035, 205036, 205037, 205038, 205039, 205040) c6( ) c7(bM, T, FR, pf, al, fd, aR, x, aE, u, K, qD, pk, aW, lW, hC, az, ih, kt, g, fT, Kv, Da, cj, k, pw, jM, NU, wu, BF, oe, jz, ec, kH, h, eV, By, hn, NX, NV, Cw, sp, zb, fe, aq, Og, BE, hk, B, Ob, fM, dA, hU, jx, eX, aA, bX, V, q, eA, NW, aT, ae, iU, oV, Y, r, lo, en, t, ck, cT, iM, iQ, wR, zA, vi, gV, aD, jh, ad, hX, l, F, NY, oW, NZ, wm, Od, ji, d, je, hG, Oc, xs, Of, ff, aF, BG, jg, iV, bz, z, bC, y, iu, jr); #c1(AIDA) c2(NP_073742) c3(axin interactor, dorsalization-associated protein) c4(213405, 213406, 213407) c5(213408, 213409, 213410, 213411, 213412, 213413, 213414, 213415, 213416, 213417, 213418, 213419, 213420, 213421, 213422, 213423, 213424, 213425) c6( ) c7(jj); #c1(AIF1) c2(NP_001614) c3(allograft inflammatory factor 1 isoform 3) c4(119219, 119220) c5(119221, 119222, 119223, 119224, 119225, 119226, 119227, 119228, 119229, 119230, 119231, 119232, 119233, 119234, 119235, 119236, 119237, 119238, 119239, 119240, 119241, 119242, 119243, 119244, 119245) c6( ) c7(cZ, T, dD, On, MH, Ol, ea, iW, oR, v, eq, uE, V, qW, eA, eW, dP, b, dV, aT, lG, Oi, dd, an, H, af, eu, Ok, ak, A, d, LC, Oj, aW, eZ, as, pb, Hv, Oh, dS, wg, jV, c, W, Om, yW, hc, i, y, cB, ao, L); #c1(AIF1) c2(NP_116573) c3(allograft inflammatory factor 1 isoform 1) c4( ) c5(226264, 226265, 226266, 226267, 226268, 226269, 226270, 226271, 226272, 226273, 226274, 226275, 226276, 226277, 226278, 226279, 226280, 226281, 226282, 226283, 226284, 226285, 226286) c6( ) c7(cZ, T, dD, On, MH, Ol, ea, iW, oR, v, eq, uE, V, qW, eA, eW, dP, b, dV, aT, lG, Oi, dd, an, H, af, eu, Ok, ak, A, d, LC, Oj, aW, eZ, as, pb, Hv, Oh, dS, wg, jV, c, W, Om, yW, hc, i, y, cB, ao, L); #c1(AIF1) c2(XP_005248928) c3(allograft inflammatory factor 1 isoform X2) c4(268782) c5(268783, 268784, 268785, 268786, 268787, 268788, 268789, 268790, 268791, 268792, 268793, 268794, 268795, 268796, 268797, 268798, 268799, 268800, 268801, 268802, 268803) c6( ) c7(cZ, T, dD, On, MH, Ol, ea, iW, OR, v, eq, uE, V, qW, eA, eW, dP, b, dV, aT, lG, Oi, dd, an, H, af, eu, Ok, ak, A, d, LC, Oj, aW, eZ, as, pb, Hv, Oh, dS, wg, jV, c, W, Om, yW, hc, i, y, cB, ao, L); #c1(AIFM1) c2(NP_001124318) c3(apoptosis-inducing factor 1, mitochondrial isoform 4) c4( ) c5(48548, 48549, 48550, 48551, 48552, 48553, 48554, 48555, 48556, 48557, 48558, 48559, 48560, 48561, 48562, 48563, 48564, 48565, 48566, 48567, 48568, 48569, 48570, 48571, 48572, 48573, 48574, 48575, 48576, 48577, 48578, 48579, 48580, 48581, 48582, 48583, 48584, 48585, 48586, 48587, 48588, 48589, 48590, 48591, 48592, 48593, 48594, 48595, 48596, 48597, 48598, 48599, 48600, 48601, 48602, 48603, 48604, 48605, 48606, 48607, 48608, 48609, 48610, 48611, 48612, 48613, 48614, 48615, 48616, 48617, 48618, 48619, 48620, 48621, 48622, 48623, 48624, 48625, 48626, 48627, 48628, 48629, 48630, 48631, 48632, 48633, 48634, 48635, 48636, 48637, 48638, 48639, 48640, 48641, 48642, 48643, 48644, 48645) c6(48646) c7(T, sw, Or, fu, eA, ad, av, aT, cy, bU, bq, ae, eu, Oq, A, B, iU, Op, bT, Y, Oo, aW, as, LK, bz, vk, rR, ac, ql, bE, bk, bt, it, X); #c1(AIFM1) c2(NP_001124319) c3(apoptosis-inducing factor 1, mitochondrial isoform 5 precursor) c4(48647, 48648) c5(48649, 48650, 48651, 48652, 48653, 48654, 48655, 48656, 48657, 48658, 48659) c6( ) c7(T, sw, Or, fu, eA, ad, av, aT, cy, bU, bq, ae, eu, Oq, A, B, iU, Op, bT, Y, Do, aW, as, LK, bz, vk, rR, ac, ql, bE, bk, bt, it, X); #c1(AIFM1) c2(NP_004199) c3(apoptosis-inducing factor 1, mitochondrial isoform 1 precursor) c4(144294, 144295, 144296) c5(144297, 144298, 144299, 144300, 144301, 144302) c6( ) c7(T, sw, Or, fu, eA, ad, av, aT, cy, bu, bq, ae, eu, Oq, A, B, iU, Op, bT, Y, Do, aW, as, LK, bz, vk, rR, ac, ql, bE, bk, bt, it, X); #c1(AIFM1) c2(NP_665811) c3(apoptosis-inducing factor 1, mitochondrial isoform 2 precursor) c4(241730, 24/731, 241732, 241733) c5(241734, 241735, 24/736, 241737, 241738, 24/739, 241740, 241741, 241742, 241743, 24/744, 241745, 241746, 241747) c6( ) c7(T, sw, Or, fu, eA, ad, av, aT, cy, bU, bq, ae, eu, Oq, A, B, iU, Op, bT, Y, Do, aW, as, LK, bz, vk, rR, ac, ql, bE, bk, bt, it, X); #c1(AIFM2) c2(NP_001185625) c3(apoptosis-inducing factor 2) c4(75997, 75998) c5(75999, 76000, 76001, 76002, 76003, 76004, 76005, 76006, 76007, 76008, 76009, 76010, 76011, 76012, 76013, 76014) c6( ) c7(t, m); #c1(AIFM3) c2(NP_001018070) c3(apoptosis-inducing factor 3 isoform 2) c4(27062, 27063, 27064) c5(27065, 27066, 27067, 27068, 27069, 27070, 27071, 27072, 27073, 27074, 27075, 27076, 27077, 27078, 27079, 27080, 27081, 27082, 27083, 27084, 27085, 27086, 27087, 27088, 27089, 27090, 27091, 27092) c6( ) c7(X); #c1(AIFM3) c2(NP_001139760) c3(apoptosis-inducing factor 3 isoform 3) c4(59901) c5(59902, 59903, 59904, 59905, 59906, 59907) c6( ) c7(X); #c1(AIFM3) c2(NP_653305) c3(apoptosis-inducing factor 3 isoform 1) c4(239538, 239539) c5(239540, 239541, 239542, 239543, 239544, 239545, 239546, 239547, 239548, 239549) c6( ) c7(X); #c1(AIG1) c2(NP_001273516) c3(androgen-induced gene 1 protein isoform b) c4( ) c5(105111, 105112, 105113, 105114, 105115, 105116) c6( ) c7(aw); #c1(AIG1) c2(NP_001273517) c3(androgen-induced gene 1 protein isoform c) c4(105117) c5(105118, 105119, 105120, 105121, 105122, 105123, 105124, 105125, 105126, 105127, 105128, 105129) c6( ) c7(aw); #c1(AIG1) c2(NP_001273518) c3(androgen-induced gene 1 protein isoform d) c4(105130) c5( ) c6( ) c7(aw); #c1(AIG1) c2(NP_057192) c3 (androgen-induced gene 1 protein isoform a) c4(189405, 189406, 189407, 189408, 189409) c5(189410, 189411, 189412, 189413, 189414, 189415, 189416, 189417, 189418, 189419, 189420, 189421, 189422, 189423, 189424, 189425, 189426, 189427) c6( ) c7(aw); #c1(AIG1) c2(XP_005267067) c3(androgen-induced gene 1 protein isoform X2) c4(280661) c5(280662, 280663, 280664, 280665, 280666, 280667) c6( ) c7(aw); #c1(AIG1) c2(XP_011534185) c3(androgen-induced gene 1 protein isoform X4) c4(330485) c5( ) c6( ) c7(aw); #c1(AIG1) c2(XP_011534186) c3(androgen-induced gene 1 protein isoform X5) c4( ) c5(330486, 330487, 330488, 330489, 330490, 330491, 330492, 330493, 330494, 330495, 330496, 330497) c6( ) c7(aw); #c1(AIMI) c2(NP_001615) c3(absent in melanoma 1 protein) c4(119246) c5(119247, 119248, 119249, 119250, 119251, 119252) c6( ) c7(ac, Y, eu, n, ae, ad, cp); #c1(AIMI) c2(XP_005266896) c3(absent in melanoma 1 protein isoform X1) c4(280376, 280377, 280378) c5(280379, 280380, 280381, 280382, 280383, 280384, 280385, 280386, 280387, 280388, 280389, 280390, 280391, 280392, 280393, 280394, 280395, 280396) c6( ) c7(ac, Y, eu, n, ae, ad, cp); #c1(AIM2) c2(NP_004824) c3(interferon-inducible protein AIM2) c4(150079, 150080, 150081) c5(150082, 150083, 150084, 150085, 150086, 150087, 150088, 150089, 150090, 150091, 150092, 150093) c6( ) c7(T, bB, ci, q, mC, ad, Ot, dV, Os, aT, Ou, ey, K, ae, d, bf, aW, Y, eZ, r, cT, pu, bE, fL); #c1(AIM2) c2(XP_005245673) c3(interferon-inducible protein AIM2 isoform X1) c4( ) c5(266876) c6( ) c7(T, bB, ci, q, mC, ad, Ot, dV, Os, aT, Ou, ey, K, ae, d, bf, aW, Y, eZ, r, cT, pu, bE, fL); #c1(AIM2) c2(XP_005245674) c3(interferon-inducible protein AIM2 isoform X2) c4( ) c5(266877, 266878, 266879, 266880, 266881) c6( ) c7(T, bB, ci, q, mC, ad, Ot, dV, Os, aT, Ou, ey, K, ae, d, bf, aW, Y, eZ, r, cT, pu, bE, fL); #c1(AIMP1) c2(NP_001135887) c3(aminoacyl tRNA synthase complex-interacting multifunctional protein 1 isoform a precursor) c4( ) c5(53499, 53500, 53501, 53502, 53503, 53504, 53505, 53506, 53507, 53508, 53509, 53510, 53511, 53512, 53513, 53514, 53515, 53516) c6( ) c7(T, dD, ea, oe, aw, eq, ci, qW, hX, aE, Ov, aT, h, an, yo, d, aW, Y, kp, fq, U, cj, uJ, y); #c1(AIMP1) c2(NP_001135888) c3(aminoacyl tRNA synthase complex-interacting multifunctional protein 1 isoform b precursor) c4(53517, 53518, 53519, 53520) c5(53521, 53522, 53523, 53524, 53525, 53526, 53527, 53528, 53529, 53530) c6( ) c7(T, dD, ea, oe, aw, eq, ci, qW, hX, aE, Ov, aT, h, an, yo, d, aW, Y, kp, fq, U, cj, uJ, y); #c1(AIMP2) c2(NP_006294) c3(aminoacyl tRNA synthase complex-interacting multifunctional protein 2) c4(163759) c5(163760, 163761, 163762, 163763, 163764, 163765) c6( ) c7(T, aC, ea, if, fd, bB, gf, lR, Lw, bv, xE, aE, rw, dV, K, aW, zC, g, as, eF, cm, bw, eM, cj, NO, Hb, m, NN, NK, dD, NJ, aj, BF, ct, qN, fr, cd, ai, Hx, jz, cU, h, dl, fe, aq, NL, w, hR, at, B, bL, Dw, NO, fM, yk, lH, jx, aA, cR, cz, v, C, V, q, eA, eJ, yw, aT, kf, cy, ae, af, hM, wr, iU, Y, r, lo, en, t, ik, pb, ck, im, bt, ao, kh, jh, dG, eq, iJ, ad, dP, av, CJ, F, ds, ji, lQ, NM, NP, d, ff, eH, cr, af, iR, bz, bE, y, iu); #c1(AIP) c2(NP_003968) c3(AH receptor-interacting protein isoform 1) c4(142509) c5(142510, 142511, 142512, 142513, 142514, 142515) c6( ) c7(T, OF, pY, Oy, lT, OE, n, DA, OH, JW, ad, hX, Do, ae, yo, Oz, BO, s, Ow, Ox, aq, OB, HI, cm, OC, OG, W, nB, iS, hR, OD, B); #c1(AIPL1) c2(NP_001028226) c3(aryl-hydrocarbon-interacting protein-like 1 isoform 2) c4(30389) c5(30390, 30391, 30392, 30393, 30394, 30395) c6( ) c7(li, lR, bP, Ol, nx, U, ab, nw, eQ, OL, Nh, OJ, OK, nC, de); #c1(AIPL1) c2(NP_001028227) c3(aryl-hydrocarbon-interacting protein-like 1 isoform 3) c4(30396) c5(30397, 30398, 30399, 30400, 30401, 30402, 30403, 30404, 30405, 30406) c6( ) c7(li, lR, bP, Ol, nx, U, ab, nw, eQ, OL, Nh, OJ, OK, nC, de); #c1(AIPL1) c2(NP_055151) c3(aryl-hydrocarbon-interacting protein-like 1 isoform 1) c4(179470, 179471) c5(179472, 179473, 179474, 179475, 179476, 179477, 179478, 179479, 179480, 179481, 179482, 179483) c6( ) c7(li, lR, bP, Ol, nx, U, ab, nw, eQ, OL, Nh, OJ, OK, nC, de); #c1(AIRE) c2(NP_000374) c3(autoimmune regulator) c4(8242, 8243, 8244, 8245) c5(8246, 8247, 8248, 8249, 8250, 8251, 8252, 8253, 8254, 8255, 8256, 8257, 8258, 8259, 8260, 8261, 8262, 8263, 8264, 8265, 8266, 8267, 8268, 8269, 8270, 8271, 8272, 8273) c6( ) c7(dA, e, aw, fN, n, OY, ON, j, ml, bX, qj, BF, bh, pd, eW, OO, xy, mP, vJ, il, nX, OP, Bh, lQ, jj, OM, hK, bc, hM, OV, A, oV, Y, OX, OQ, hl, OS, OZ, kt, eZ, aF, OR, zZ, f, OU, dr, pb, mk, OW, Pa, Lj, OT, CZ, yH, bD, hE, kS, y, pC); #c1(AIRE) c2(XP_011527853) c3(autoimmune regulator isoform X1) c4( ) c5(322815) c6( ) c7(dA, e, aw, fN, n, OY, ON, j, ml, bX, qj, BF, bh, pd, eW, OO, xy, mP, vJ, il, nX, OP, Bh, lQ, jj, OM, hk, bc, hM, OV, A, oV, Y, OX, OQ, hl, OS, OZ, kt, eZ, aF, OR, zZ, f, OU, dr, pb, mk, OW, Pa, Lj, OT, CZ, yH, bD, hE, kS, y, pC); #c1(AJAP1) c2(XP_011540088) c3(adherens junction-associated protein 1 isoform X1) c4(338428) c5(338429, 338430, 338431, 338432, 338433, 338434, 338435, 338436, 338437, 338438) c6( ) c7(ka, d, ki, De, aA, aj, cm, Fc, eA); #c1(AJUBA) c2(NP_116265) c3(LIM domain-containing protein ajuba isoform 1) c4(226034, 226035, 226036, 226037) c5(226038, 226039, 226040, 226041, 226042, 226043, 226044, 226045, 226046, 226047, 226048, 226049, 226050, 226051) c6( ) c7(it, q, r, aT); #c1(AJUBA) c2(NP_932352) c3(LIM domain-containing protein ajuba isoform 2) c4( ) c5(258979, 258980, 258981, 258982, 258983, 258984, 258985, 258986, 258987, 258988, 258989, 258990, 258991, 258992, 258993, 258994, 258995, 258996) c6( ) c7(it, q, r, aT); #c1(AKI) c2(NP_000467) c3(adenylate kinase isoenzyme 1) c4(9286) c5(9287, 9288, 9289, 9290, 9291, 9292, 9293, 9294, 9295, 9296) c6( ) c7(bL, wi, sF, Pb, j, S, Pc); #c1(AK3) c2(NP_001186781) c3(GTP:AMP phosphotransferase AK3, mitochondrial isoform b) c4(78565) c5(78566, 78567, 78568, 78569, 78570, 78571) c6( ) c7(NK, ag, qh, oh, dX, Pd); #c1(AK3) c2(NP_001186785) c3(GTP:AMP phosphotransferase AK3, mitochondrial isoform c) c4( ) c5(78572, 78573, 78574, 78575, 78576, 78577, 78578, 78579) c6( ) c7(NK, ag, qh, oh, dx, Pd); #c1(AK3) c2(NP_057366) c3(GTP:AMP phosphotransferase AK3, mitochondrial isoform a) c4(190380, 190381) c5(190382, 190383, 190384, 190385, 190386, 190387, 190388, 190389) c6( ) c7(NK, ag, qh, oh, dX, Pd); #c1(AKG) c2(NP_001015891) c3(adenylate kinase isoenzyme 6 isoform c) c4(26245) c5(26246, 26247, 26248, 26249, 26250, 26251) c6( ) c7(LB, sb, pc, iT, cj, Pe, dz, K, ra, hF, ae, ad); #c1(AKG) c2(NP_057367) c3(adenylate kinase isoenzyme 6 isoform b) c4(190390) c5(190391, 190392, 190393, 190394, 190395, 190396) c6( ) c7(LB, sb, pc, iT, cj, Pe, dz, K, ra, hF, ae, ad); #c1(AK7) c2(NP_689540) c3 (adenylate kinase 7) c4(243034) c5(243035, 243036, 243037, 243038, 243039, 243040) c6( ) c7(Pf, MG); #c1(AK8) c2(NP_689785) c3(adenylate kinase 8) c4(243946) c5(243947, 243948, 243949, 243950, 243951, 243952) c6( ) c7(fr); #c1(AK9) c2(NP_659462) c3(adenylate kinase 9 isoform 2) c4( ) c5(240064, 240065, 240066, 240067, 240068, 240069, 240070, 240071, 240072, 240073, 240074, 240075) c6( ) c7(vi); #c1(AK9) c2(XP_006715439) c3(adenylate kinase 9 isoform X2) c4(289559) c5(289560, 289561, 289562, 289563, 289564, 289565) c6( ) c7(vi); #c1(AK9) c2(XP_011533856) c3(adenylate kinase 9 isoform X5) c4(330087) c5( ) c6( ) c7(vi); #c1(AK9) c2(XP_011533858) c3(adenylate kinase 9 isoform X7) c4( ) c5(330088) c6( ) c7(vi); #c1(AK9) c2(XP_011533859) c3(adenylate kinase 9 isoform X8) c4(330089) c5( ) c6( ) c7(vi); #c1(AK9) c2(XP_011533860) c3(adenylate kinase 9 isoform X9) c4( ) c5(330090, 330091, 330092, 330093, 330094) c6( ) c7(vi); #c1(AK9) c2(XP_011533861) c3(adenylate kinase 9 isoform X10) c4(330095, 330096) c5(330097, 330098, 330099, 330100, 330101, 330102) c6( ) c7(vi); #c1(AKAP1) c2(NP_003479) c3(A-kinase anchor protein 1, mitochondrial precursor) c4(138677, 138678) c5(138679, 138680, 138681, 138682, 138683, 138684, 138685, 138686, 138687, 138688, 138689, 138690, 138691, 138692, 138693, 138694) c6( ) c7(cF, aW, at, v, ae, eu, ad); #c1(AKAP1) c2(XP_011523620) c3(A-kinase anchor protein 1, mitochondrial isoform X2) c4(318107) c5(318108, 318109, 318110, 318111, 318112, 318113) c6( ) c7(cF, aW, aT, v, ae, eu, ad); #c1(AKAP10) c2(NP_009133) c3(A-kinase anchor protein 10, mitochondrial precursor) c4(171001) c5(171002, 171003, 171004, 171005, 171006, 171007) c6( ) c7(eR, aW, aw, cz, r, aT, K, ei, q); #c1(AKAP10) c2(XP_006721495) c3(A-kinase anchor protein 10, mitochondrial isoform X2) c4(294003) c5(294004, 294005, 294006, 294007, 294008, 294009) c6( ) c7(eR, aW, aw, cz, r, aT, K, ei, q); #c1(AKAPI2) c2(NP_005091) c3(A-kinase anchor protein 12 isoform 1) c4(152819) c5(152820, 152821, 152822, 152823, 152824, 152825, 152826, 152827, 152828, 152829) c6( ) c7(G, T, pf, ht, aA, ci, hF, q, ad, aT, hu, ae, eG, iU, ff, r, kp, af, en, bw, ac, bP, dy, bt, it, B); #c1(AKAPI2) c2(NP_653080) c3(A-kinase anchor protein 12 isoform 2) c4(238824) c5(238825, 238826, 238827, 238828, 238829, 238830) c6( ) c7(G, T, pf, ht, aA, ci, hF, q, ad, aT, hu, ae, eG, iU, ff, r, kp, aF, en, bw, ac, bP, dy, bt, it, B); #c1(AKAP12) c2(XP_005267292) c3 (A-kinase anchor protein 12 isoform X2) c4(280875) c5(280876, 280877, 280878, 280879, 280880, 280881) c6( ) c7(G, T, pf, ht, aA, ci, hF, q, ad, aT, hu, ae, eG, iU, ff, r, kp, aF, en, bw, ac, bP, dy, bt, it, B); #c1(AKAP13) c2(NP_001257475) c3(A-kinase anchor protein 13 isoform 4) c4(95477) c5(95478, 95479, 95480, 95481, 95482, 95483, 95484, 95485, 95486, 95487, 95488) c6( ) c7(dP, bM, T, iV, ra, aW, hF, ht, Pg, r, aT, fe, bw, fr, m, bu, t, bt, a, q, ad); #c1(AKAP13) c2(NP_006729) c3(A-kinase anchor protein 13 isoform 1) c4(167492, 167493, 167494, 167495, 167496, 167497) c5(167498, 167499, 167500, 167501, 167502, 167503, 167504, 167505, 167506, 167507, 167508, 167509, 167510, 167511, 167512, 167513, 167514, 167515, 167516, 167517, 167518, 167519, 167520, 167521, 167522, 167523, 167524, 167525) c6( ) c7(dP, bM, T, iV, ra, aW, hF, ht, Pg, r, aT, fe, bw, fr, m, bu, t, bt, ae, q, ad); #c1(AKAP13) c2(NP_009131) c3(A-kinase anchor protein 13 isoform 2) c4(170970, 170971, 170972, 170973) c5(170974, 170975, 170976, 170977, 170978, 170979, 170980, 170981, 170982, 170983, 170984, 170985, 170986, 170987, 170988, 170989, 170990, 170991, 170992, 170993, 170994, 170995, 170996, 170997, 170998, 170999, 171000) c6( ) c7(dP, bM, T, iV, ra, aW, hF, ht, Pg, r, aT, fe, bw, fr, m, bu, t, bt, ae, q, ad); #c1(AKAPI7A) c2(NP_005079) c3(A-kinase anchor protein 17A isoform 1) c4(152706, 152707) c5(152708, 152709, 152710, 152711, 152712, 152713, 152714, 152715, 152716, 152717, 152718, 152719, 152720) c6( ) c7(T, fM); #c1(AKAP2) c2(NP_001004065) c3(A-kinase anchor protein 2 isoform 1) c4(20415) c5(20416, 20417, 20418, 20419, 20420, 20421) c6( ) c7(ff, ht, en); #c1(AKAP2) c2(NP_001185585) c3(A-kinase anchor protein 2 isoform 3) c4(75965) c5(75966, 75967, 75968, 75969, 75970, 75971) c6( ) c7(ff, ht, en); #c1(AKAP3) c2(NP_001265238) c3(A-kinase anchor protein 3) c4( ) c5(98026, 98027, 98028, 98029, 98030, 98031) c6( ) c7(t, n, m, bu); #c1(AKAP3) c2(XP_011519212) c3(A-kinase anchor protein 3 isoform X1) c4(313791, 313792) c5(313793, 313794, 313795, 313796, 313797, 313798, 313799, 313800, 313801, 313802) c6( ) c7(t, n, m, bu); #c1(AKAP4) c2(NP_003877) c3(A-kinase anchor protein 4 isoform 1) c4(141719, 14/720, 14/721, 141722, 14/723) c5(141724, 141725, 141726, 141727, 141728, 141729, 141730, 141731, 141732, 141733, 141734, 141735, 141736, 141737, 141738, 141739, 141740, 141741, 141742) c6( ) c7(aW, if, aT, Ph, qN, bu, in); #c1(AKAP4) c2(NP_647450) c3(A-kinase anchor protein 4 isoform 2) c4( ) c5(238602, 238603, 238604, 238605, 238606, 238607, 238608, 238609, 238610, 238611, 238612, 238613, 238614, 238615, 238616, 238617, 238618, 238619) c6( ) c7(aW, if, aT, Ph, qN, bu, in); #c1(AKAP5) c2(NP_004848) c3(A-kinase anchor protein 5) c4(150292) c5(150293, 150294, 150295, 150296, 150297, 150298, 150299, 150300, 150301, 150302) c6( ) c7(zB, ot, Pi); #c1(AKAP6) c2(NP_004265) c3(A-kinase anchor protein 6) c4(144712, 144713) c5(144714, 144715, 144716, 144717, 144718, 144719, 144720) c6( ) c7(bM, cF, wK, iX, lr, j, fq); #c1(AKAP6) c2(XP_005268276) c3(A-kinase anchor protein 6 isoform X1) c4( ) c5(281660, 281661, 281662, 281663, 281664, 281665, 281666, 281667, 281668) c6( ) c7(bM, cF, wK, iX, lr, j, fq); #c1(AKAP7) c2(NP_004833) c3(A-kinase anchor protein 7 isoform gamma isoform alpha) c4(150151) c5(150152, 150153, 150154, 150155, 150156, 150157) c6( ) c7(vr, lB, cd, bA); #c1(AKAP7) c2(NP_057461) c3(A-kinase anchor protein 7 isoform gamma isoform gamma) c4(191047, 191048) c5(191049, 191050, 191051, 191052, 191053, 191054, 191055, 191056, 191057, 191058, 191059, 191060) c6( ) c7(vr, IB, cd, bA); #c1(AKAP7) c2(NP_619539) c3(A-kinase anchor protein 7 isoform gamma isoform beta) c4(236867) c5(236868, 236869, 236870, 236871, 236872, 236873) c6( ) c7(vr, lB, cd, bA); #c1(AKAP9) c2(NP_005742) c3(A-kinase anchor protein 9 isoform 2) c4(158967) c5(158968, 158969, 158970, 158971, 158972, 158973) c6( ) c7(T, hd, Pj, nB, db, aW, aT, r, vc, lQ, Pk, q, mu); #c1(AKAP9) c2(XP_011514014) c3(A-kinase anchor protein 9 isoform X4) c4(306426) c5( ) c6( ) c7(T, hd, Pj, nB, db, aW, aT, r, vc, lQ, Pk, q, mu); #c1(AKAP9) c2(XP_011514018) c3(A-kinase anchor protein 9 isoform X7) c4( ) c5(306427) c6( ) c7(T, hd, Pj, nB, db, aW, aT, r, vc, lQ, Pk, q, mu); #c1(AKAP9) c2(XP_011514020) c3(A-kinase anchor protein 9 isoform X9) c4(306428, 306429) c5(306430, 306431, 306432, 306433, 306434, 306435, 306436, 306437, 306438, 306439, 306440, 306441) c6( ) c7(T, hd, Pj, nB, db, aW, aT, r, vc, lQ, Pk, q, mu); #c1(AKAP9) c2(XP_011514021) c3(A-kinase anchor protein 9 isoform X10) c4(306442) c5(306443, 306444, 306445, 306446, 306447) c6( ) c7(T, hd, Pj, nB, db, aW, aT, r, vc, lQ, Pk, q, mu); #c1(AKAP9) c2(XP_011514022) c3(A-kinase anchor protein 9 isoform X12) c4( ) c5(306448, 306449, 306450, 306451, 306452, 306453) c6( ) c7(T, hd, Pj, nB, db, aW, aT, r, vc, lQ, Pk, q, mu); #c1(AKAP9) c2(XP_011514024) c3(A-kinase anchor protein 9 isoform X14) c4(306454) c5(306455, 306456, 306457, 306458, 306459, 306460) c6( ) c7(T, hd, Pj, nB, db, aW, aT, r, vc, lO, Pk, q, mu); #c1(AKIP1) c2(NP_001193575) c3(A-kinase-interacting protein 1 isoform b) c4(82264) c5(82265, 82266, 82267, 82268, 82269, 82270, 82271, 82272, 82273, 82274) c6( ) c7(aW, if, Pl, qN, aT); #c1(AKIP1) c2(NP_001193576) c3(A-kinase-interacting protein 1 isoform c) c4(82275) c5(82276, 82277, 82278, 82279, 82280, 82281) c6( ) c7(aW, if, Pl, qN, aT); #c1(AKIP1) c2(NP_065693) c3(A-kinase-interacting protein 1 isoform a) c4(204986) c5(204987, 204988, 204989, 204990, 204991, 204992, 204993, 204994, 204995, 204996) c6( ) c7(aW, if, Pl, qN, aT); #c1(AKIRIN2) c2(NP_060534) c3(akirin-2) c4(196448) c5(196449, 196450, 196451, 196452, 196453, 196454) c6( ) c7(DI, ff, aq, en, y, a, B, Pm, ad); #c1(AKNA) c2(NP_110394) c3(AT-hook-containing transcription factor) c4(220014, 220015) c5(220016, 220017, 220018, 220019, 220020, 220021, 220022, 220023, 220024, 220025, 220026, 220027, 220028, 220029, 220030, 220031, 220032) c6( ) c7(T, if, qN); #c1(AKNA) c2(XP_005252305) c3(AT-hook-containing transcription factor isoform X2) c4(271654) c5( ) c6( ) c7(T, if, qN); #c1(AKNA) c2(XP_006717358) c3(AT-hook-containing transcription factor isoform X10) c4( ) c5(290973, 290974, 290975, 290976, 290977, 290978) c6( ) c7(T, if, qN); #c1(AKNA) c2(XP_011517362) c3(AT-hook-containing transcription factor isoform X3) c4( ) c5(311090) c6( ) c7(T, if, qN); #c1(AKNA) c2(XP_011517364) c3(AT-hook-containing transcription factor isoform X5) c4(311091) c5(311092, 311093, 311094, 311095, 311096) c6( ) c7(T, if, qN); #c1(AKNA) c2(XP_011517366) c3(AT-hook-containing transcription factor isoform X7) c4(311097) c5( ) c6( ) c7(T, if, qN); #c1(AKNA) c2(XP_011517367) c3(AT-hook-containing transcription factor isoform X8) c4(311098) c5( ) c6( ) c7(T, if, qN); #c1(AKRIB1) c2(NP_001619) c3(aldose reductase) c4(119273, 119274) c5(119275, 119276, 119277, 119278, 119279, 119280, 119281, 119282, 119283, 119284, 119285, 119286) c6( ) c7(bM, T, ea, if, e, bB, ci, aY, x, Pq, fm, iv, Pn, K, de, aW, mQ, cm, vA, vy, m, k, cZ, dD, cQ, aj, qN, pM, cd, ft, ju, dd, vx, A, iw, aq, Pp, op, kT, FV, hL, Pr, Po, B, tq, bL, aA, CR, cz, v, j, bX, eA, J, uT, aT, cy, bq, s, ev, cl, t, ho, ck, cT, ac, fP, dy, fD, bt, HQ, gV, Pt, dG, eq, ah, fu, fU, ts, Ps, tt, ux, lR, hG, aF, iR, bz, DF, bE, aS, ra, X, hv); #c1(AKR1B10) c2(NP_064695) c3(aldo-keto reductase family 1 member B10) c4(203473) c5(203474, 203475, 203476, 203477, 203478, 203479) c6( ) c7(Kd, T, dD, pf, if, bB, eq, ci, qN, q, Pu, eJ, av, iu, Pv, F, aE, jz, aT, K, Pw, s, Gv, Y, ff, iw, LZ, r, aq, kp, en, cl, g, LV, iR, bz, eM, bE, vy, hL, dy, cP, B); #c1(AKR1C1) c2(NP_001344) c3(aldo-keto reductase family 1 member CI) c4(117150, 117151, 117152) c5(117153, 117154, 117155, 117156, 117157, 117158, 117159, 117160, 117161, 117162, 117163, 117164, 117165, 117166, 117167, 117168, 117169, 117170, 117171, 117172, 117173, 117174, 117175, 117/76, 117/77, 117178) c6( ) c7(dP, av, T, kl, eM, vT, aW, aZ, iW, ff, hL, aT, r, gE, m, q, en, Px, bz, B, c1); #c1(AKR1C2) c2(NP_001128713) c3(aldo-keto reductase family 1 member C2 isoform 2) c4(50572) c5(50573, 50574, 50575, 50576, 50577, 50578) c6( ) c7(T, pf, v, Pz, ad, dP, Py, aT, dL, ae, kl, vT, aW, ff, PA, NH, LZ, aq, en, g, iR, Px, eM, it, B); #c1(AKR1C2) c2(NP_001345) c3(aldo-keto reductase family 1 member C2 isoform 1) c4(117179) c5(117180, 117181, 117182, 117183, 117184, 117185, 117186, 117187, 117188, 117189, 117190, 117191, 117192) c6( ) c7(T, pf, v, Pz, ad, dP, Py, aT, dL, ae, kl, vT, aW, ff, PA, NH, LZ, aq, en, g, iR, Px, eM, it, B); #c1(AKR1C2) c2(XP_011517644) c3(aldo-keto reductase family 1 member C2 isoform X1) c4(311484) c5(311485, 311486, 311487, 311488, 311489, 311490) c6( ) c7(T, pf, v, Pz, ad, dP, Py, aT, dL, ae, kl, vT, aW, ff, PA, NH, LZ, aq, en, g, iR, Px, eM, it, B); #c1(AKR1C3) c2(NP_001240837) c3(aldo-keto reductase family 1 member C3 isoform 2) c4(88924) c5(88925, 88926, 88927, 88928, 88929, 88930) c6( ) c7(G, T, aZ, if, iW, aA, n, v, oH, ci, Fb, qN, AO, PB, eA, ad, PC, dP, av, Bj, aT, iv, PD, dL, K, uV, ae, eL, kl, iU, vT, aW, ff, cr, dN, dq, en, cl, g, t, Px, bz, as, eE, ck, kP, JQ, hl, at, k, bt); #c1(AKR1C3) c2(NP_001240838) c3(aldo-keto reductase family 1 member C3 isoform 3) c4( ) c5(88931, 88932, 88933, 88934, 88935, 88936) c6( ) c7(G, T, aZ, if, iW, aA, n, v, oH, ci, Fb, qN, AD, PB, eA, ad, PC, dP, av, Bj, aT, iv, PD, dL, K, uV, ae, eL, kl, iU, vT, aW, ff, cr, dN, dq, en, cl, g, t, Px, bz, as, eE, ck, kP, JQ, hL, at, k, bt); #c1(AKR1C3) c2(NP_003730) c3(aldo-keto reductase family 1 member C3 isoform 1) c4(140487) c5(140488, 140489, 140490, 140491, 140492, 140493, 140494, 140495, 140496) c6( ) c7(G, T, aZ, if, iW, aA, n, v, oH, ci, Fb, qN, AO, PB, eA, ad, PC, dP, av, Bj, aT, iv, PD, dL, K, uV, ae, eL, kl, iU, vT, aW, ff, cr, dN, dq, en, cl, g, t, Px, bz, as, BE, ck, kP, JQ, hL, at, k, bt); #c1(AKR1C4) c2(NP_001809) c3(aldo-keto reductase family 1 member C4) c4(121304, 121305) c5(121306, 121307, 121308, 121309, 121310, 121311, 121312, 121313, 121314, 121315, 121316, 121317, 121318, 121319, 121320, 121321, 121322, 121323, 121324, 121325, 121326, 121327, 121328, 121329, 121330, 121331, 121332, 121333) c6( ) c7(oO, T, aw, fd, br, ad, Py, sO, aT, h, dL, bK, ae, el, MG, PE, aW, lo, PG, pm, bw, PF, ac, pc, cj, at, rH, bt, L); #c1(AKR1C4) c2(XP_011517605) c3(aldo-keto reductase family 1 member C4 isoform X1) c4(311396, 311397) c5( ) c6( ) c7(oO, T, aw, fd, br, ad, Py, sO, aT, h, dL, bK, ae, eL, MG, PE, aW, lo, PG, pm, bw, PF, ac, pc, cj, at, rH, bt, L); #c1(AKR1D1) c2(NP_001177835) c3(3-oxo-5-beta-steroid 4-dehydrogenase isoform 2) c4(73075) c5(73076, 73077, 73078, 73079, 73080, 73081) c6( ) c7(yy, PH); #c1(AKR1D1) c2(NP_001177836) c3(3-oxo-5-beta-steroid 4-dehydrogenase isoform 3) c4(73082, 73083) c5(73084, 73085, 73086, 73087, 73088, 73089, 73090, 73091, 73092, 73093, 73094, 73095) c6( ) c7(yy, PH); #c1(AKR1D1) c2(NP_005980) c3(3-oxo-5-beta-steroid 4-dehydrogenase isoform 1) c4(160953) c5(160954, 160955, 160956, 160957, 160958, 160959) c6( ) c7(yy, PH); #c1(AKR1E2) c2(NP_001035267) c3(1,5-anhydro-D-fructose reductase isoform 1) c4(32726) c5(32727, 32728, 32729, 32730, 32731, 32732) c6( ) c7(aR, Pl, B, gP, vT); #c1(AKR1E2) c2(NP_001257950) c3(1,5-anhydro-D-fructose reductase isoform 2) c4(95843, 95844) c5(95845, 95846, 95847, 95848, 95849, 95850, 95851, 95852, 95853, 95854, 95855, 95856) c6( ) c7(aR, PI, B, gP, vT); #c1(AKR1E2) c2(NP_001257954) c3(1,5-anhydro-D-fructose reductase isoform 3) c4(95865) c5(95866, 95867, 95868, 95869, 95870, 95871) c6( ) c7(aR, PI, B, gP, vT); #c1(AKR7A3) c2(XP_011539349) c3(aflatoxin BI aldehyde reductase member 3 isoform X2) c4( ) c5(337437) c6( ) c7(T); #c1(AKT1) c2(NP_001014431) c3(RAC-alpha serine/threonine-protein kinase) c4(25825, 25826) c5(25827, 25828, 25829, 25830, 25831, 25832, 25833, 25834, 25835, 25836, 25837, 25838, 25839, 25840, 25841, 25842, 25843, 25844, 25845, 25846, 25847, 25848, 25849, 25850, 25851, 25852, 25853, 25854, 25855, 25856, 25857, 25858, 25859, 25860, 25861) c6( ) c7(oO, gh, T, lD, aC, fR, ea, if, al, fd, bB, ci, Qp, QG, aR, ip, x, bv, aE, Bc, fm, pX, Fo, Qk, iv, DO, HR, K, hu, pk, eL, xa, cp, Qe, li, ly, xl, yo, zQ, PP, iE, LC, aW, Ls, az, hl, QB, Qv, Qq, CT, Qb, Qm, PQ, g, as, eF, cm, bw, Qx, Qt, On, QA, iP, qJ, eM, nB, iA, iT, go, vA, mx, U, cj, PV, Qw, sv, nw, PO, m, uJ, QI, QF, ik, iu, dD, Qj, ht, qy, n, aj, oY, fr, PY, Qh, PJ, qN, kv, PR, cd, ai, eW, co, Bl, ft, jz, hw, cc, zf, gd, kH, h, jy, QE, dl, bl, eV, Qc, dx, WC, PT, MD, PX, hQ, aq, kp, yq, CQ, pm, PU, cM, fs, wU, op, lx, cv, Ld, iO, ok, Qy, Qf, cA, bP, dB, hk, Qu, hL, hR, at, Az, it, B, AN, vQ, bL, fM, As, yk, jx, eX, rf, aA, cR, zN, cz, v, j, yO, Qo, lu, AO, pd, q, J, qH, eA, eJ, PW, lg, hW, PS, nv, Qg, BJ, yg, aT, kf, cy, bK, jj, an, ch, ae, af, eu, jf, kl, PK, s, vT, Qr, oV, Y, PL, iU, r, lo, HL, en, hf, bu, t, ho, f, pb, cl, bN, iK, ck, Qi, sS, rR, kP, c, ac, ig, nM, iM, au, dh, mf, dy, PN, im, bt, KG, LS, On, nZ, hl, kh, id, Mb, qb, ha, Me, jh, KO, bS, dG, eq, Fb, hF, ad, hX, Jm, dP, HF, hd, ib, av, py, db, NS, oj, hD, bj, iY, fb, PZ, Qd, uX, PM, d, je, cW, ff, eH, cr, gl, aF, iR, jb, bz, Hp, bF, es, gk, eU, Qa, bE, X, ll, QC, QD, cP, bC, ei, y, xg, ne, jr); #c1(AKT1) c2(XP_011534846) c3(RAC-alpha serine/threonine-protein kinase isoform X2) c4(331362) c5(331363, 331364, 331365, 331366, 331367, 331368) c6( ) c7(oO, gh, T, lD, aC, fR, ea, if, al, fd, bB, ci, Qp, QG, aR, ip, x, bv, aE, Bc, fm, pX, Fo, Ok, iv, DO, HR, K, hu, pk, eL, xa, cp, de, li, ly, xQ, yo, zQ, PP, iE, LC, aW, Ls, az, hl, QB, Qv, Qq, CT, Qb, Qm, PQ, g, as, eF, cm, bw, Qx, Qt, Qn, QA, iP, qJ, eM, nB, iA, iT, go, vA, mx, U, cj, PV, Qw, sv, nw, PO, m, uJ, QI, QF, ik, iu, dD, Qj, ht, qy, n, aj, oY, fr, PY, Ch, PJ, qN, kv, PR, cd, ai, eW, co, Bl, ft, jz, hw, cc, zf, gd, KH, h, jy, QE, dL, bl, eV, Qc, dx, wC, PT, MD, PX, hQ, aq, kp, yq, CQ, pm, PU, cM, fs, wU, op, lx, cv, Ld, iO, ok, By, Of, cA, bP, dB, hk, Qu, hL, hR, at, Az, it, B, AN, vQ, bL, fM, Qs, yk, jx, eX, rf, aA, CR, zN, cz, v, j, yO, No, lu, AO, pd, q, J, qH, eA, eJ, PW, lg, hW, PS, nv, Qg, BJ, yg, aT, kf, cy, bK, jj, an, ch, ae, af, eu, jf, kl, PK, s, vT, Ar, oV, Y, PL, iU, r, lo, HL, en, hf, bu, t, ho, f, pb, cl, bN, iK, ck, Qi, sS, rR, kP, c, ac, ig, nM, iM, au, dh, mf, dy, PN, im, bt, KG, LS, On, nZ, hl, kh, id, Mb, qb, ha, Me, jh, KO, bS, dG, eq, Fb, hF, ad, hX, Jm, dP, HF, hd, ib, av, py, db, NS, oj, HD, bj, iY, fb, PZ, Qd, ux, PM, d, je, cW, ff, eH, cr, gl, aF, iR, jb, bz, Hp, bF, es, gk, eU, Qa, bE, X, ll, QC, QD, cP, bC, ei, y, xg, ne, jr); #c1(AKT1S1) c2(NP_001092103) c3(proline-rich AKT1 substrate 1 isoform b) c4( ) c5(39355, 39356, 39357, 39358, 39359, 39360, 39361, 39362, 39363, 39364, 39365, 39366, 39367, 39368, 39369, 39370, 39371, 39372, 39373, 39374, 39375, 39376) c6( ) c7(es, iA, Y, aw, lG, eA, dx, ek); #c1(AKT1S1) c2(NP_115751) c3(proline-rich AKT1 substrate 1 isoform a) c4(223816, 223817, 223818) c5(223819, 223820, 223821, 223822, 223823, 223824, 223825, 223826, 223827, 223828, 223829, 223830) c6( ) c7(es, iA, Y, aw, lG, eA, dx, ek); #c1(AKT2) c2(NP_001229957) c3(RAC-beta serine/threonine-protein kinase isoform 2) c4(85533) c5(85534, 85535, 85536, 85537, 85538, 85539) c6( ) c7(bL, gh, T, dD, fM, aC, qb, aZ, ea, jh, aA, cz, bB, aj, dG, v, ci, Qh, eq, eA, J, ad, QI, fu, iu, ft, LL, hw, aE, Bc, QJ, ju, aT, h, cy, jy, dL, bK, K, fb, ae, eL, QH, yo, kl, d, aW, Y, Ls, AQ, ff, Qq, dq, kp, aF, en, cl, g, t, cm, bz, GR, sS, QG, eM, nB, bE, bo, dh, X, hL, QC, m, ei, B); #c1(AKT2) c2(XP_011524923) c3(RAC-beta serine/threonine-protein kinase isoform X1) c4(319321, 319322, 319323, 319324, 319325, 319326, 319327, 319328, 319329, 319330) c5(319331, 319332, 319333, 319334, 319335, 319336, 319337, 319338, 319339, 319340, 319341, 319342, 319343, 319344, 319345, 319346) c6( ) c7(bL, gh, T, dD, fM, aC, qb, aZ, ea, jh, aA, cz, bB, aj, dG, v, ci, Qh, eq, eA, J, ad, QI, fU, iu, ft, LL, hw, aE, Bc, QJ, ju, aT, h, cy, jy, dL, bK, K, fb, ae, eL, QH, yo, kl, d, aW, Y, Ls, AQ, ff, Qq, dq, kp, aF, en, cl, g, t, cm, bz, GR, sS, QG, eM, nB, bE, bo, dh, X, hL, QC, m, ei, B); #c1(AKT2) c2(XP_011524924) c3(RAC-beta serine/threonine-protein kinase isoform X2) c4(319347) c5(319348, 319349, 319350, 319351, 319352, 319353, 319354, 319355, 319356, 319357, 319358, 319359, 319360, 319361, 319362, 319363, 319364, 319365, 319366, 319367, 319368, 319369, 319370, 319371, 319372, 319373, 319374, 319375, 319376, 319377, 319378, 319379, 319380, 319381, 319382, 319383, 319384, 319385, 319386, 319387, 319388, 319389, 319390, 319391, 319392, 319393, 319394, 319395, 319396, 319397, 319398, 319399, 319400, 319401, 319402, 319403, 319404, 319405) c6( ) c7(bL, gh, T, dD, fM, aC, qb, aZ, ea, jh, aA, cz, bB, aj, dG, v, ci, Qh, eq, eA, J, ad, QI, fU, iu, ft, LL, hw, aE, Bc, QJ, ju, aT, h, cy, jy, dL, bK, K, fb, ae, eL, QH, yo, kl, d, aW, Y, Ls, AQ, ff, Qq, dq, kp, aF, en, cl, g, t, cm, bz, GR, sS, QG, eM, nB, bE, bo, dh, X, hL, QC, m, ei, B); #c1(AKT3) c2(NP_001193658) c3(RAC-gamma serine/threonine-protein kinase isoform 2) c4(82419) c5(82420, 82421, 82422, 82423, 82424, 82425, 82426, 82427, 82428, 82429) c6( ) c7(gh, T, ha, EQ, QL, fr, PY, ci, PJ, V, eA, ad, hw, pN, aT, h, dL, ae, ud, PM, d, aW, Y, ff, Qq, aq, en, iR, t, QK, kP, c, nB, Dy, eM, FB, Gs, bo, m, B); #c1(AKT3) c2(XP_006725023) c3(RAC-gamma serine/threonine-protein kinase isoform X1) c4(296526) c5(296527, 296528, 296529, 296530, 296531, 296532, 296533, 296534, 296535, 296536) c6( ) c7(gh, T, ha, EQ, QL, fr, PY, ci, PJ, V, eA, ad, hw, pN, aT, h, dL, ae, ul, PM, d, aW, Y, ff, Qq, aq, en, iR, t, QK, kP, c, nB, Dy, eM, FB, Gs, bo, m, B); #c1(AKT3) c2(XP_006725026) c3(RAC-gamma serine/threonine-protein kinase isoform X7) c4( ) c5(296537, 296538, 296539, 296540, 296541, 296542) c6( ) c7(gh, T, ha, EQ, QL, fr, PY, ci, PJ, V, eA, ad, hw, pN, aT, h, dL, ae, ul, PM, d, aW, Y, ff, Qq, aq, en, iR, t, OK, kP, c, nB, Dy, eM, FB, Gs, bo, m, B); #c1(AKT3) c2(XP_011544728) c3(RAC-gamma serine/threonine-protein kinase isoform X5) c4(343807) c5( ) c6( ) c7(gh, T, ha, EQ, QL, fr, PY, ci, PJ, V, eA, ad, hw, pN, aT, h, dL, ae, ul, PM, d, aW, Y, ff, Qq, aq, en, iR, t, QK, kP, c, nB, Dy, eM, FB, Gs, bo, m, B); #c1(AKT1P) c2(NP_071921) c3(AKT-interacting protein) c4(212317) c5(212318, 212319, 212320, 212321, 212322, 212323, 212324, 212325, 212326, 212327) c6( ) c7(QO, if, QM, bK, ci, qN, t, QN); #c1(ALAD) c2(NP_000022) c3(delta-aminolevulinic acid dehydratase) c4(211) c5(212, 213, 214, 215, 216) c6( ) c7(T, QR, fR, QW, cz, ww, fr, eA, ad, ip, PW, Gt, QU, dl, bq, ae, B, tb, Ow, QP, OC, hq, c, dB, QQ, yP, Pk, QV, QT, at, E, dy, gT, cB, QS); #c1(ALAD) c2(XP_01516665) c3(delta-aminolevulinic acid dehydratase isoform X1) c4( ) c5(310076, 310077, 310078, 310079, 310080, 310081, 310082) c6( ) c7(T, QR, fR, QW, cz, ww, fr, eA, ad, ip, PW, Gt, QU, dl, bq, ae, B, tb, Ow, QP, OC, hq, c, dB, QQ, yP, Pk, QV, QT, at, E, dy, gT, cB, QS); #c1(ALAD) c2(XP_011516666) c3(delta-aminolevulinic acid dehydratase isoform X3) c4(310083, 310084) c5(310085, 310086, 310087, 310088, 310089, 310090, 310091, 310092, 310093, 310094) c6( ) c7(T, QR, fR, QW, cz, ww, fr, eA, ad, ip, PW, Gt, QU, dl, bq, ae, B, tb, Ow, QP, OC, hq, c, dB, QQ, yP, Pk, QV, QT, at, E, dy, gT, cB, QS); #c1(ALAS1) c2(NP_001291372) c3(5-aminolevulinate synthase, nonspecific, mitochondrial isoform 1 precursor) c4(115762, 115763) c5(115764, 115765, 115766, 115767, 115768, 115769, 115770, 115771, 115772, 115773, 115774, 115775, 115776, 115777, 115778) c6( ) c7(QX, kA, QV, aq, aj, C, bq, nE, lC, J, ae, B, ad); #c1(ALAS1) c2(NP_001291373) c3(5-aminolevulinate synthase, nonspecific, mitochondrial isoform 2) c4( ) c5(115779, 115780, 115781, 115782) c6( ) c7(QX, kA, QV, aq, aj, C, bq, nE, lC, J, ae, B, ad); #c1(ALAS1) c2(XP_011531780) c3(5-aminolevulinate synthase, nonspecific, mitochondrial isoform X2) c4( ) c5(327867, 327868, 327869, 327870, 327871) c6( ) c7(QX, kA, QV, aq, aj, C, bq, nE, lC, J, ae, B, ad); #c1(ALAS2) c2(NP_000023) c3(5-aminolevulinate synthase, erythroid-specific, mitochondrial isoform a precursor) c4(217, 218) c5(219, 220, 221, 222, 223, 224) c6( ) c7(jp, kA, QZ, nE, Rc, Ra, nd, oW, yu, K, hl, QY, nf, KX, Rb, kD, CE, S, yy, dr, Gq, Rd, kB, kg, kz, Lq); #c1(ALAS2) c2(NP_001033056) c3(5-aminolevulinate synthase, erythroid-specific, mitochondrial isoform b precursor) c4(31531) c50) c6( ) c7(jp, kA, QZ, nE, Rc, Ra, nd, oW, yu, K, hl, QY, nf, KX, Rb, kD, CE, S, yy, dr, Gq, Rd, KB, kg, kz, Lq); #c1(ALAS2) c2(NP_001033057) c3(5-aminolevulinate synthase, erythroid-specific, mitochondrial isoform c precursor) c4(31532) c5(31533, 31534, 31535, 31536, 31537, 31538, 31539, 31540, 31541, 31542, 31543, 31544) c6( ) c7(jp, kA, QZ, nE, Rc, Ra, nd, oW, yu, K, hl, QY, nf, KX, Rb, KD, CE, S, yy, dr, Gq, Rd, kB, kg, kz, Lq); #c1(ALAS2) c2(XP_005262052) c3(5-aminolevulinate synthase, erythroid-specific, mitochondrial isoform X1) c4( ) c5(277728, 277729, 277730, 277731, 277732, 277733) c6( ) c7(jp, kA, QZ, nE, Rc, Ra, nd, oW, yu, K, hl, QY, nf, KX, Rb, kD, CE, S, yy, dr, Gq, Rd, kB, kg, kz, Lq); #c1(ALB) c2(NP_000468) c3(serum albumin preproprotein) c4(9297, 9298, 9299, 9300, 9301, 9302, 9303, 9304) c5(9305, 9306, 9307, 9308, 9309, 9310, 9311, 9312, 9313, 9314, 9315, 9316, 9317, 9318, 9319, 9320, 9321, 9322, 9323, 9324, 9325, 9326, 9327, 9328, 9329, 9330, 9331, 9332, 9333, 9334, 9335, 9336, 9337, 9338, 9339, 9340, 9341, 9342, 9343, 9344, 9345, 9346, 9347, 9348, 9349, 9350, 9351, 9352, 9353, 9354, 9355, 9356, 9357, 9358, 9359, 9360, 9361, 9362, 9363, 9364, 9365, 9366, 9367, 9368, 9369, 9370, 9371, 9372, 9373, 9374, 9375, 9376, 9377, 9378, 9379, 9380, 9381, 9382, 9383, 9384, 9385, 9386, 9387, 9388, 9389, 9390, 9391, 9392, 9393, 9394, 9395, 9396, 9397, 9398, 9399, 9400, 9401, 9402, 9403, 9404, 9405, 9406, 9407, 9408, 9409, 9410, 9411, 9412, 9413, 9414, 9415, 9416) c6( ) c7(xS, T, ef, Rz, fV, ea, e, al, fd, bB, ci, aY, fw, RL, lv, Bc, iv, K, wt, de, H, RG, bc, Rw, Rh, aW, RK, Rr, hl, mQ, fc, dg, g, eE, y, ii, dc, vy, wx, m, Re, fy, Rg, Ro, dD, Rx, RC, Ri, fN, n, BH, cd, aj, ct, uE, fr, qW, cd, RD, eW, co, jz, no, pe, ec, Ru, VM, ql, RN, CU, lG, jy, h, DU, yZ, RM, bV, RA, db, RE, cY, A, Rj, iw, fl, aq, RI, jT, cM, Rn, ar, iO, ok, RB, W, dB, Fi, lf, mg, RJ, E, va, B, tq, L, Rf, bL, dA, eR, dK, jx, Rt, aA, cz, fF, Ry, v, j, hz, OY, nS, V, uW, q, J, RI, uT, hb, aT, GQ, cy, af, eu, kl, s, vT, oV, Y, ev, RF, lo, wA, Rp, t, ck, cl, ac, vW, hc, DW, mf, dy, vi, Ke, ob, RH, ha, dG, ah, jE, fu, eq, pz, tK, Rk, hX, fU, F, Rs, Rq, od, oz, ds, qh, oj, oW, vw, uD, fb, sj, uU, uX, lR, cH, yy, aF, xH, iV, iR, HI, KM, dr, qa, LB, gk, Fd, Rv, ag, uR, gZ, Rm, dX, aS, ei, hE, we, iu, rk); #c1(ALCAM) c2(NP_001230209) c3(CD166 antigen isoform 2 precursor) c4(86117) c5(86118, 86119, 86120, 86121, 86122, 86123) c6( ) c7(T, ht, NT, aA, bB, n, aj, ci, zP, V, AO, q, J, ad, eJ, nv, aT, dL, hu, ae, de, zQ, d, s, aW, Y, ff, r, aq, aF, en, iR, t, ik, iP, bF, nM, eM, nB, dB, bE, m, it, iu, hS); #c1(ALCAM) c2(NP_001618) c3(CD166 antigen isoform 1 precursor) c4(119253, 119254, 119255) c5(119256, 119257, 119258, 119259, 119260, 119261, 119262, 119263, 119264, 119265, 119266, 119267, 119268, 119269, 119270, 119271, 119272) c6( ) c7(T, ht, NT, aA, bB, n, aj, ci, zP, V, AO, q, J, ad, eJ, nv, aT, dl, hu, ae, de, zQ, d, s, aW, Y, ff, r, aq, aF, en, iR, t, ik, iP, bF, nM, eM, nB, dB, bE, m, it, iu, hS); #c1(ALDH16A1) c2(NP_001138868) c3(aldehyde dehydrogenase family 16 member A1 isoform 2) c4(57963) c5(57964, 57965, 57966, 57967, 57968, 57969, 57970) c6( ) c7(RO, nD, nH); #c1(ALDH16A1) c2(NP_699160) c3(aldehyde dehydrogenase family 16 member A1 isoform 1) c4(245728, 245729, 245730) c5(245731, 245732, 245733, 245734, 245735, 245736, 245737, 245738, 245739, 245740, 245741, 245742, 245743, 245744, 245745, 245746, 245747, 245748) c6( ) c7(RO, nD, nH); #c1(ALDH18A1) c2(NP_001017423) c3(delta-l-pyrroline-5-carboxylate synthase isoform 2) c4(26479) c5(26480, 26481, 26482, 26483, 26484, 26485) c6( ) c7(RQ, RR, BN, RP, uo, dq, hy, ne); #c1(ALDH18AI) c2(NP_002851) c3(delta-l-pyrroline-5-carboxylate synthase isoform 1) c4(132786) c5(132787, 132788, 132789, 132790, 132791, 132792) c6( ) c7(RQ, RR, BN, RP, uo, dq, hy, ne); #c1(ALDH1A1) c2(NP_000680) c3(retinal dehydrogenase 1) c4(14425, 14426, 14427, 14428, 14429) c5(14430, 14431, 14432, 14433, 14434, 14435, 14436, 14437, 14438, 14439, 14440, 14441, 14442, 14443, 14444, 14445, 14446, 14447, 14448, 14449, 14450, 14451, 14452, 14453, 14454, 14455, 14456, 14457, 14458, 14459) c6( ) c7(T, aC, dA, zX, ht, NT, fd, n, aj, v, fr, ci, bX, lu, q, J, ad, GK, RU, ft, hd, aE, aT, cy, K, hu, ae, RS, xl, RT, MD, aW, hl, r, fQ, aq, g, cl, eE, t, cm, bz, ik, op, ck, RV, kP, eM, nB, vA, Gs, RW, PV, Rm, Lb, m, ik, B); #c1(ALDH1A2) c2(NP_001193826) c3(retinal dehydrogenase 2 isoform 4) c4(82695) c5(82696, 82697, 82698, 82699, 82700, 82701, 82702) c6( ) c7(Mg, jW, rq, s, nB, nO, RX, cz, bD, n, ti, Fx, K, m, ci, dy, ae, ad, lU); #c1(ALDH1A2) c2(NP_003879) c3(retinal dehydrogenase 2 isoform 1) c4(141750, 141751, 141752, 141753, 141754) c5(141755, 141756, 141757, 141758, 141759, 141760, 14/761, 14/762, 141763, 14/764, 141765, 141766, 14/767, 141768, 14/769, 14/770, 14/771, 141772, 14/773, 141774, 141775, 141776, 141777, 14/778) c6( ) c7(Mg, jW, rq, s, nB, nO, RX, cz, bD, n, ti, Fx, K, m, ci, dy, ae, ad, lU); #c1(ALDH1A2) c2(NP_733797) c3(retinal dehydrogenase 2 isoform 2) c4(247139, 247140) c5(247141, 247142, 247143, 247144, 247145, 247146, 247147, 247148) c6( ) c7(Mg, jW, rq, s, nB, nO, RX, cz, bD, n, ti, Fx, K, m, ci, dy, ae, ad, lU); #c1(ALDH1A2) c2(NP_733798) c3(retinal dehydrogenase 2 isoform 3) c4( ) c5(247149, 247150, 247151, 247152, 247153, 247154, 247155, 247156, 247157, 247158) c6( ) c7(Mg, jW, rq, s, nB, nO, RX, cz, bD, n, ti, Fx, K, m, ci, dy, ae, ad, lU); #c1(ALDH1A3) c2(NP_000684) c3(aldehyde dehydrogenase family 1 member A3 isoform 1) c4(14526, 14527) c5(14528, 14529, 14530, 14531, 14532, 14533, 14534, 14535, 14536, 14537, 14538, 14539) c6( ) c7(T, d, kj, RY, nO, aW, Y, aZ, eL, aT, aA, n, RZ, dL, bD, bz); #c1(ALDH1B1) c2(NP_000683) c3(aldehyde dehydrogenase X, mitochondrial precursor) c4(14512, 14513) c5(14514, 14515, 14516, 14517, 14518, 14519, 14520, 14521, 14522, 14523, 14524, 14525) c6( ) c7(fQ, aj, fS, j); #c1(ALDH1L1) c2(NP_001257293) c3(cytosolic 10-formyltetrahydrofolate dehydrogenase isoform 1) c4(95081, 95082, 95083, 95084, 95085) c5(95086, 95087, 95088, 95089, 95090, 95091, 95092, 95093, 95094) c6( ) c7(T, fM, iW, oe, v, ad, Na, av, M, aT, Nc, dL, K, r, fQ, as, bz, Hp, ac, eM, lp, at); #c1(ALDH1L1) c2(NP_001257294) c3(cytosolic 10-formyltetrahydrofolate dehydrogenase isoform 3) c4(95095, 95096) c5(95097, 95098, 95099, 95100, 95101, 95102, 95103, 95104, 95105, 95106, 95107) c6( ) c7(T, fM, iW, oe, v, ad, Na, av, M, aT, Nc, dL, K, r, fQ, as, bz, Hp, ac, eM, lp, at); #c1(ALDH1L1) c2(NP_036322) c3(cytosolic 10-formyltetrahydrofolate dehydrogenase isoform 2) c4(172989) c5(172990, 172991, 172992, 172993, 172994, 172995, 172996, 172997, 172998, 172999, 173000, 173001, 173002, 173003, 173004, 173005, 173006, 173007, 173008) c6( ) c7(T, fM, iW, oe, v, ad, Na, av, M, aT, Nc, dL, K, r, fQ, as, bz, Hp, ac, eM, lp, at); #c1(ALDH1L1) c2(XP_011510658) c3(cytosolic 10-formyltetrahydrofolate dehydrogenase isoform X2) c4(301094) c5(301095, 301096, 301097, 301098, 301099, 301100, 301101, 301102, 301103, 301104, 301105, 301106, 301107, 301108, 301109, 301110, 301111, 301112, 301113) c6( ) c7(T, fM, iW, oe, v, ad, Na, av, M, aT, Nc, dl, K, r, fQ, as, bz, Hp, ac, eM, lp, at); #c1(ALDH2) c2(NP_000681) c3(aldehyde dehydrogenase, mitochondrial isoform 1 precursor) c4(14460) c5(14461, 14462, 14463, 14464, 14465, 14466) c6( ) c7(Gk, bM, T, os, uk, aC, ea, Sg, e, al, ci, aE, Bc, fS, Gb, Gr, K, hu, oi, Se, aW, az, hl, md, fc, kt, da, aB, g, ba, fT, nF, Gs, mx, vy, cX, dD, gU, aw, ht, NT, n, aj, cd, md, FY, Gm, cc, jy, Gw, dL, wb, Go, RS, hi, ny, rk, FZ, A, Sa, fQ, aq, Gi, ar, Sc, Gq, Sk, at, uz, te, bD, jd, it, B, bL, eR, oe, He, aA, cz, hy, v, j, Ge, q, J, Mg, Si, qp, aT, cy, bK, an, af, bA, Sh, s, bg, r, eS, Sf, en, Sj, SI, KC, ik, bN, tR, ac, Cz, gX, mf, cS, np, oX, vq, GI, ob, Gd, oZ, bS, dG, fu, eq, iG, Sd, tK, ad, kL, av, nH, ib, yt, NS, Sb, na, Jc, BN, lR, Gv, ff, Sn, aF, Sm, bz, qa, D, gk, wz, gZ, aS, tr, ei, Gu, Gp, zk, vt, X, us); #c1(ALDH2) c2(NP_001191818) c3(aldehyde dehydrogenase, mitochondrial isoform 2 precursor) c4(81841) c5(81842, 81843, 81844, 81845, 81846, 81847) c6( ) c7(Gk, bM, T, os, uk, aC, ea, Sg, e, al, ci, aE, Bc, fS, Gb, Gr, K, hu, oi, Se, aW, az, hl, md, fc, kt, da, aB, g, ba, fT, nF, Gs, mx, vy, cx, dD, gU, aw, ht, NT, n, aj, cd, md, FY, Gm, cc, jy, Gw, dL, wb, Go, RS, hi, ny, rk, FZ, A, Sa, fQ, aq, Gi, ar, Sc, Gq, Sk, at, uz, te, bD, jd, it, B, bL, eR, oe, He, aA, cz, hy, v, j, Ge, q, J, Mg, Si, qp, aT, cy, bK, an, af, bA, Sh, s, bg, r, eS, Sf, en, Sj, SI, kC, iK, bN, tR, ac, Cz, gX, mf, cS, np, oX, vq, Gl, ob, Gd, oZ, bS, dG, fu, eq, iG, Sd, tK, ad, KL, av, nH, ib, yt, NS, Sb, na, Jc, BN, lR, Gv, ff, Sn, aF, Sm, bz, qa, D, gk, wz, gZ, aS, tr, ei, Gu, Gp, zk, vt, X, us); #c1(ALDH3AI) c2(NP_000682) c3(aldehyde dehydrogenase, dimeric NADP-preferring) c4(14467, 14468, 14469, 14470, 14471, 14472) c5(14473, 14474, 14475, 14476, 14477, 14478, 14479, 14480, 14481, 14482, 14483, 14484, 14485, 14486, 14487, 14488, 14489, 14490, 14491, 14492, 14493, 14494, 14495, 14496, 14497, 14498, 14499, 14500, 14501, 14502, 14503, 14504, 14505, 14506, 14507, 14508, 14509, 14510, 14511) c6( ) c7(av, T, iu, d, eM, aT, n, aq, Lb, ci, im, t, ae, B, ad, bN); #c1(ALDH3A1) c2(XP_011522033) c3(aldehyde dehydrogenase, dimeric NADP-preferring isoform X2) c4( ) c5(316710, 316711, 316712, 316713, 316714) c6( ) c7(av, T, iu, d, eM, aT, n, aq, Lb, ci, im, t, ae, B, ad, bN); #c1(ALDH3A2) c2(NP_000373) c3(fatty aldehyde dehydrogenase isoform 2) c4(8225) c5(8226, 8227, 8228, 8229, 8230, 8231, 8232, 8233, 8234, 8235, 8236, 8237, 8238, 8239, 8240, 8241) c6( ) c7(cF, Gy, W, lg, Sr, KA, So, fn, Sq, K, nA, Ss, Gz, Sp, nC, vN, Nh, hX); #c1(ALDH3A2) c2(NP_001026976) c3(fatty aldehyde dehydrogenase isoform 1) c4(30025, 30026) c5(30027, 30028, 30029, 30030, 30031, 30032) c6( ) c7(CF, Gy, W, lg, Sr, KA, So, fn, Sq, K, nA, Ss, Gz, Sp, nC, vN, Nh, hX); #c1(ALDH3BI) c2(NP_000685) c3(aldehyde dehydrogenase family 3 member B1 isoform a) c4( ) c5(14540, 14541) c6( ) c7(bL, qi, fr, K); #c1(ALDH4A1) c2(XP_011540654) c3(delta-l-pyrroline-5-carboxylate dehydrogenase, mitochondrial isoform X1) c4(339213) c5(339214, 339215, 339216, 339217, 339218, 339219) c6( ) c7(St); #c1(ALDH4A1) c2(XP_011540655) c3(delta-1-pyrroline-5-carboxylate dehydrogenase, mitochondrial isoform X2) c4(339220) c5(339221, 339222, 339223, 339224, 339225, 339226, 339227, 339228, 339229, 339230, 339231, 339232, 339233, 339234, 339235, 339236, 339237, 339238, 339239, 339240, 339241, 339242, 339243) c6( ) c7(St); #c1(ALDHSA1) c2(NP_733936) c3(succinate-semialdehyde dehydrogenase, mitochondrial isoform 1 precursor) c4(247285) c5(247286, 247287, 247288, 247289, 247290, 247291) c6( ) c7(Sw, nX, GD, ha, aU, Sz, Sv, K, nA, Sx, Sy, dr, xt, Su); #c1(ALDH6AI) c2(NP_005580) c3(methylmalonate-semialdehyde dehydrogenase [acylating], mitochondrial isoform 1 precursor) c4(157622) c5(157623, 157624, 157625, 157626, 157627, 157628) c6( ) c7(rL, v, SA, j, SB); #c1(ALDH7A1) c2(NP_001173) c3(alpha-aminoadipic semialdehyde dehydrogenase isoform 1 precursor) c4(72416) c5(72417, 72418, 72419, 72420, 72421, 72422) c6( ) c7(bL, bM, T, lD, Dd, ha, bO, aA, cz, n, bS, ct, Lw, ad, BY, zw, aT, SD, he, ae, bA, aW, Fm, Y, nk, Z, nC, bz, Ld, lx, ck, kn, cT, rR, eM, SE, bo, SC, pP, cj, U, nw, fD); #c1(ALDH7A1) c2(NP_001189333) c3(alpha-aminoadipic semialdehyde dehydrogenase isoform 3) c4(79276) c5(79277, 79278, 79279, 79280, 79281, 79282) c6( ) c7(bL, bM, T, lD, Dd, ha, bO, aA, cz, n, bS, ct, Lw, ad, BY, zw, aT, SD, he, ae, bA, aW, Fm, Y, nk, Z, nC, bz, Ld, lx, ck, kn, cT, rR, eM, SE, bo, SC, pP, cj, U, nw, fD); #c1(ALDH7A1) c2(XP_011541719) c3(alpha-aminoadipic semialdehyde dehydrogenase isoform X1) c4(340271) c5(340272, 340273, 340274, 340275, 340276, 340277) c6( ) c7(bL, bM, T, lD, Dd, ha, bO, aA, cz, n, bS, ct, Lw, ad, BY, zw, aT, SD, he, ae, bA, aW, Fm, Y, nk, Z, nC, bz, Ld, lx, ck, kn, cT, rR, eM, SE, bo, SC, pP, cj, U, nw, fD); #c1(ALDHSA1) c2(NP_000687) c3(4-trimethylaminobutyraldehyde dehydrogenase) c4(14542, 14543) c5(14544, 14545, 14546, 14547, 14548, 14549, 14550) c6( ) c7(fr, cz, bA, cv); #c1(ALDHSA1) c2(XP_011507596) c3(4-trimethylaminobutyraldehyde dehydrogenase isoform X1) c4( ) c5(297506, 297507, 297508, 297509, 297510) c6( ) c7(fr, cz, bA, cv); #c1(ALDOA) c2(NP_001230106) c3(fructose-bisphosphate aldolase A isoform 2) c4(85920, 85921) c5(85922, 85923, 85924, 85925, 85926, 85927, 85928, 85929, 85930, 85931, 85932, 85933, 85934, 85935, 85936, 85937) c6(85938) c7(yt, ld, eH, oW, aT, SF, lG, j, CI, jo, eF, iu, bN); #c1(ALDOA) c2(NP_908930) c3(fructose-bisphosphate aldolase A isoform 1) c4( ) c5(258109, 258110, 258111, 258112, 258113, 258114, 258115, 258116, 258117, 258118, 258119, 258120, 258121, 258122, 258123, 258124, 258125, 258126, 258127, 258128, 258129, 258130, 258131, 258132, 258133, 258134, 258135, 258136, 258137, 258138, 258139, 258140, 258141, 258142, 258143, 258144, 258145, 258146, 258147, 258148, 258149, 258150, 258151, 258152, 258153, 258154, 258155, 258156, 258157, 258158, 258159, 258160, 258161, 258162, 258163, 258164, 258165, 258166, 258167, 258168, 258169, 258170, 258171, 258/72, 258173, 258174, 258175, 258176, 258177, 258178, 258179, 258180, 258181, 258182, 258183, 258184, 258185, 258186, 258187, 258188, 258189, 258190, 258191, 258192, 258193, 258194, 258195, 258196, 258197, 258198, 258199, 258200, 258201, 258202, 258203, 258204, 258205, 258206, 258207, 258208, 258209, 258210, 258211) c6( ) c7(yt, ld, eH, oW, aT, SF, lG, j, Cl, jo, eF, iu, bN); #c1(ALDOB) c2(NP_000026) c3(fructose-bisphosphate aldolase B) c4(246, 247, 248) c5(249, 250, 251, 252, 253, 254, 255, 256, 257, 258, 259, 260, 261, 262, 263, 264, 265, 266) c6( ) c7(bL, iV, ht, jx, aA, aq, SG, bh, bq, B); #c1(ALDOC) c2(NP_005156) c3(fructose-bisphosphate aldolase C) c4(153466, 153467) c5(153468, 153469, 153470, 153471, 153472, 153473, 153474, 153475, 153476, 153477, 153478, 153479, 153480, 153481, 153482, 153483) c6( ) c7(cm, SH, B); #c1(ALG1) c2(NP_061982) c3(chitobiosyldiphosphodolichol beta-mannosyltransferase) c4(201609, 201610) c5(201611, 201612, 201613, 201614, 201615, 201616, 201617) c6( ) c7(bL, eM, aW, iW, dv, aT, aU, Km, PJ, SI, X, dS); #c1(ALG1) c2(XP_011520867) c3(chitobiosyldiphosphodolichol beta-mannosyltransferase isoform X1) c4( ) c5(315886, 315887, 315888, 315889, 315890) c6( ) c7(bL, eM, aW, iW, dv, aT, aU, Km, PJ, SI, X, dS); #c1(ALG10) c2(NP_116223) c3(dol-P-Glc:Glc(2)Man(9)GlcNAc(2)-PP-Dol alpha-1,2-glucosyltransferase) c4(225851) c5(225852, 225853, 225854, 225855, 225856, 225857) c6( ) c7(SJ, Mk, af, mu); #c1(ALG10B) c2(NP_001013642) c3(putative Dol-P-Glc:Glc(2)Man(9)GlcNAc(2)-PP-Dol alpha-1,2-glucosyltransferase) c4(25639) c5(25640, 25641, 25642, 25643, 25644, 25645) c6( ) c7(Mk, mu); #c1(ALG11) c2(NP_001004127) c3(GDP-Man:Man(3)GlcNAc(2)-PP-Dol alpha-1,2-mannosyltransferase) c4(20448, 20449) c5(20450, 20451, 20452, 20453, 20454, 20455, 20456, 20457, 20458, 20459, 20460) c6( ) c7(SK, SL); #c1(ALG12) c2(NP_077010) c3(dol-P-Man:Man(7)GlcNAc(2)-PP-Dol alpha-1,6-mannosyltransferase) c4(214729) c5(214730, 214731, 214732, 214733, 214734, 214735, 214736, 214737, 214738, 214739, 214740) c6( ) c7(SN, Lo, Km, SM); #c1(ALG13) c2(NP_001093392) c3 (putative bifunctional UDP-N-acetylglucosamine transferase and deubiquitinase ALG13 isoform 1) c4(40258) c5(40259, 40260, 40261, 40262, 40263, 40264) c6( ) c7(lm, SO); #c1(ALG13) c2(NP_001244163) c3(putative bifunctional UDP-N-acetylglucosamine transferase and deubiquitinase ALG13 isoform 5) c4( ) c5(92020, 92021, 92022, 92023, 92024) c6( ) c7(lm, SO); #c1(ALG13) c2(NP_060936) c3(putative bifunctional UDP-N-acetylglucosamine transferase and deubiquitinase ALG13 isoform 2) c4(198777) c5(198778, 198779, 198780, 198781, 198782, 198783) c6( ) c7(lm, SO); #c1(ALG13) c2(XP_006724758) c3(putative bifunctional UDP-N-acetylglucosamine transferase and deubiquitinase ALG13 isoform X6) c4(296323) c5(296324) c6( ) c7(lm, SO); #c1(ALG1L) c2(NP_001015050) c3(putative glycosyltransferase ALG1-like isoform 2) c4( ) c5(26152, 26153, 26154, 26155, 26156) c6( ) c7(iR, aq, cd); #c1(ALG1L) c2(NP_001182152) c3(putative glycosyltransferase ALG1-like isoform 1) c4( ) c5(74623) c6( ) c7(iR, aq, cd); #c1(ALG1L) c2(XP_011510831) c3(putative glycosyltransferase ALG1-like isoform X1) c4(301293) c5( ) c6( ) c7(iR, aq, cd); #c1(ALG2) c2(NP_149078) c3(alpha-1,3/1,6-mannosyltransferase ALG2) c4(227034, 227035) c5(227036, 227037, 227038, 227039, 227040, 227041, 227042, 227043, 227044, 227045, 227046, 227047) c6( ) c7(xA, T, Y, SP); #c1(ALG3) c2(NP_001006942) c3(dol-P-Man:Man(5)GlcNAc(2)-PP-Dol alpha-1,3-mannosyltransferase isoform d) c4(22359) c5(22360, 22361, 22362, 22363, 22364, 22365, 22366, 22367, 22368, 22369, 22370, 22371) c6( ) c7(Lo, SQ, SL); #c1(ALG3) c2(NP_005778) c3(dol-P-Man:Man(5)GlcNAc(2)-PP-Dol alpha-1,3-mannosyltransferase isoform a) c4(159260) c5(159261, 159262, 159263, 159264, 159265, 159266) c6( ) c7(Lo, SQ, SL); #c1(ALG3) c2(XP_011510624) c3(dol-P-Man:Man(5)GlcNAc(2)-PP-Dol alpha-1,3-mannosyltransferase isoform X1) c4(301082) c5( ) c6( ) c7(Lo, SQ, SL); #c1(ALG8) c2(NP_001007028) c3(probable dolichyl pyrophosphate GlclMan9GlcNAc2 alpha-1,3-glucosyltransferase isoform b) c4(22436) c5(22437, 22438, 22439, 22440, 22441) c6( ) c7(bL, Lo, SR); #c1(ALG8) c2(NP_076984) c3(probable dolichyl pyrophosphate GlcMan9GlcNAc2 alpha-1,3-glucosyltransferase isoform a) c4(214593) c5(214594, 214595, 214596, 214597, 214598, 214599, 214600) c6( ) c7(bL, Lo, SR); #c1(ALG9) c2(NP_001071158) c3(alpha-1,2-mannosyltransferase ALG9 isoform b) c4(35320, 35321) c5(35322, 35323, 35324, 35325, 35326, 35327, 35328) c6( ) c7(SS, yL, gl, K, dv); #c1(ALG9) c2(NP_001071159) c3(alpha-1,2-mannosyltransferase ALG9 isoform c precursor) c4( ) c5(35329, 35330, 35331, 35332, 35333) c6( ) c7(SS, yL, gl, K, dv); #c1(ALG9) c2(NP_001071160) c3(alpha-1,2-mannosyltransferase ALG9 isoform d precursor) c4( ) c5(35334, 35335, 35336, 35337, 35338) c6( ) c7(SS, yL, gl, K, dv); #c1(ALG9) c2(NP_079016) c3(alpha-1,2-mannosyltransferase ALG9 isoform a) c4(216592) c5(216593) c6( ) c7(SS, yL, gl, K, dv); #c1(ALK) c2(NP_004295) c3(ALK tyrosine kinase receptor precursor) c4(144934, 144935) c5(144936, 144937, 144938, 144939, 144940, 144941, 144942, 144943, 144944, 144945, 144946, 144947, 144948, 144949, 144950, 144951) c6( ) c7(T, aC, Tb, bB, ci, x, bv, aE, Td, Tm, pX, SV, K, iE, aW, Om, g, cm, Qx, Qt, eM, nB, cj, zv, k, m, Th, n, aj, fr, pU, TI, SW, b, ft, Tf, ju, Nn, h, eV, hn, dx, Fp, MD, aq, kp, Ca, fq, Qy, Of, bP, dB, Qu, Tk, SZ, Te, B, fM, ex, fZ, ST, j, V, q, eA, aT, Tj, ae, ne, jf, Or, NE, iU, oV, Y, xR, r, en, t, ck, Ta, sS, nM, iM, QG, iW, jh, Gd, SY, hF, ad, Tg, av, hd, fx, SU, Ti, lQ, d, je, ff, kD, zL, bz, SX, i, iu, jr, Tc); #c1(ALKBH1) c2(NP_006011) c3(alkylated DNA repair protein alkB homolog 1) c4(161144) c5(161145, 161146, 161147, 161148, 161149, 161150) c6( ) c7(T, aR, id, Om, eL, mA, e, az, jN, wf, aj, dl, aB, ci, ln, qW, J, dR); #c1(ALKBH2) c2(NP_001001655) c3 (alpha-ketoglutarate-dependent dioxygenase alkB homolog 2 isoform 1) c4(18890, 18891, 18892, 18893) c5(18894, 18895, 18896, 18897, 18898, 18899, 18900, 18901, 18902, 18903, 18904, 18905, 18906, 18907, 18908, 18909, 18910, 18911, 18912, 18913, 18914, 18915, 18916, 18917, 18918, 18919, 18920, 18921, 18922, 18923, 18924, 18925) c6( ) c7(eL, Bl, dL, id, ci); #c1(ALKBH2) c2(NP_001192109) c3(alpha-ketoglutarate-dependent dioxygenase alkB homolog 2 isoform 2) c4(81917) c5(81918, 81919, 81920, 81921, 81922, 81923) c6( ) c7(eL, Bl, dL, id, ci); #c1(ALKBH3) c2(NP_631917) c3(alpha-ketoglutarate-dependent dioxygenase alkB homolog 3) c4(238335, 238336, 238337) c5(238338, 238339, 238340, 238341, 238342, 238343, 238344, 238345, 238346, 238347, 238348, 238349, 238350, 238351, 238352, 238353, 238354, 238355) c6( ) c7(ae, T, id, aE, eM, eL, ff, oW, dV, fd, n, dl, ci, en, g, bz, iu, ad, zn); #c1(ALKBH7) c2(NP_115682) c3(alpha-ketoglutarate-dependent dioxygenase alkB homolog 7, mitochondrial precursor) c4(223534) c5(223535, 223536, 223537, 223538, 223539, 223540) c6( ) c7(GE); #c1(ALKBH8) c2(NP_001287939) c3(alkylated DNA repair protein alkB homolog 8 isoform 1) c4(113247) c5(113248, 113249, 113250, 113251, 113252, 113253, 113254, 113255, 113256, 113257, 113258, 113259, 113260, 113261, 113262, 113263) c6( ) c7(BI, T, id, dL, ci, el); #c1(ALKBH8) c2(NP_620130) c3 (alkylated DNA repair protein alkB homolog 8 isoform 2) c4(237323) c5( ) c6( ) c7(BI, T, id, dL, ci, el); #c1(ALKBH8) c2(XP_005271789) c3(alkylated DNA repair protein alkB homolog 8 isoform X4) c4( ) c5(283591, 283592, 283593, 283594, 283595, 283596) c6( ) c7(BI, T, id, dl, ci, el); #c1(ALKBH8) c2(XP_011541369) c3(alkylated DNA repair protein alkB homolog 8 isoform X3) c4(339981) c5( ) c6( ) c7(BI, T, id, dl, ci, el); #c1(ALLC) c2(NP_060906) c3(probable allantoicase) c4(198573) c5(198574, 198575, 198576, 198577, 198578, 198579) c6( ) c7(uk, Y, jh, cj, h, To); #c1(ALMS1) c2(NP_055935) c3(Alstrom syndrome protein 1) c4(184518, 184519) c5(184520, 184521, 184522, 184523, 184524, 184525, 184526, 184527, 184528, 184529, 184530, 184531, 184532) c6( ) c7(bL, A, cc, lR, eR, aw, mx, aj, v, Tp, Tq, J); #c1(ALOX12) c2(NP_000688) c3(arachidonate 12-lipoxygenase, 12S-type) c4(14551) c5(14552, 14553, 14554, 14555, 14556, 14557) c6( ) c7(bL, bM, T, dD, ea, e, aw, NT, aA, cz, v, eq, ci, bX, fr, cd, q, ad, aT, lG, dl, bK, lQ, is, ae, af, eu, bA, lR, s, aW, Y, Tr, yx, ff, r, af, en, bu, t, cm, qa, ac, hk, at, gv, m, y, bt, it); #c1(ALOX12B) c2(NP_001130) c3(arachidonate 12-lipoxygenase, 12R-type) c4(52509) c5( ) c6( ) c7(ac, lt, Ts, e, aw, ff, aT, fn, j, m, bz, en, fq, eu); #c1(ALOX15) c2(NP_001131) c3(arachidonate 15-lipoxygenase) c4(52637, 52638) c5( ) c6( ) c7(bL, bM, T, dD, aC, eX, ea, e, ht, NT, oA, aA, cz, bB, aj, v, j, ci, uE, bX, eq, hF, AO, fr, q, J, cd, ad, dP, ai, av, aw, dY, uT, aE, sN, sO, M, aT, h, cy, dz, dL, eV, K, hu, is, ae, af, eu, bA, A, lR, s, et, iU, LC, sp, Y, aW, bg, r, aF, g, t, cm, bz, ck, ba, es, bF, ac, bE, wO, hc, hk, dh, at, m, y, in, it, X, ao); #c1(ALOX15B) c2(NP_001034219) c3 (arachidonate 15-lipoxygenase B isoform a) c4(31588) c5(31589, 31590, 31591, 31592, 31593, 31594) c6( ) c7(ae, G, T, dD, cF, s, ea, ht, r, aT, iv, eq, m, hu, lC, ik, bz, q, ad); #c1(ALOX15B) c2(NP_001034220) c3(arachidonate 15-lipoxygenase B isoform b) c4(31595) c5(31596, 31597, 31598, 31599, 31600, 31601, 31602, 31603, 31604, 31605) c6( ) c7(ae, G, T, dD, cF, s, ea, ht, r, aT, iv, eq, m, hu, lC, iK, bz, q, ad); #c1(ALOX5) c2(NP_000689) c3(arachidonate 5-lipoxygenase isoform 1) c4(14558) c5(14559, 14560, 14561, 14562, 14563, 14564) c6( ) c7(ID, bM, T, aC, fR, tQ, ea, e, al, l, fd, bB, ci, aY, Tz, dY, aE, Bc, bW, M, Ds, K, dQ, aW, g, as, eF, ou, U, m, R, dD, aw, ht, NT, aj, pM, fK, ai, ec, Lv, lG, eV, dL, sp, lb, fe, aq, dB, JM, bm, at, E, B, vQ, bL, Dw, eR, oe, eX, aA, cz, v, j, C, q, eA, cF, aT, cy, jj, ae, af, eu, bf, s, iU, Tv, r, bu, t, pb, ck, rR, nM, ac, Ty, c, ig, hc, gX, fP, Da, cS, Tu, dy, fD, bt, wi, uG, jh, ah, eq, ad, BY, hX, yt, oz, Tx, ji, jo, gj, d, lR, eH, si, TA, es, gk, Bv, bE, uR, aS, Tw, lC, KS, X, y); #c1(ALOX5) c2(NP_001243083) c3(arachidonate 5-lipoxygenase isoform 3) c4(89743) c5(89744, 89745, 89746, 89747, 89748, 89749) c6( ) c7(ID, bM, T, aC, fR, tQ, ea, e, al, l, fd, bB, ci, aY, Tz, dY, aE, Bc, bW, M, Ds, K, dQ, aW, g, as, eF, ou, U, m, R, dD, aw, ht, NT, aj, pM, fK, ai, ec, Lv, lG, eV, dl, sp, lb, fe, aq, dB, JM, bm, at, E, B, vQ, bL, Dw, eR, oe, eX, aA, cz, v, j, C, q, eA, cF, aT, cy, jj, ae, af, eu, bf, s, iU, Tv, r, bu, t, pb, ck, rR, nM, ac, Ty, c, ig, hc, gX, fP, Da, cS, Tu, dy, fD, bt, wi, uG, jh, ah, eq, ad, BY, hX, yt, oz, Tx, ji, jo, gj, d, lR, eH, si, TA, es, gk, Bv, bE, uR, aS, Tw, lC, KS, X, y); #c1(ALOX5AP) c2(NP_001191335) c3(arachidonate 5-lipoxygenase-activating protein isoform 2) c4( ) c5(81419) c6( ) c7(bM, dD, ea, e, aw, NT, cz, OR, bS, j, ct, v, eq, zP, TB, Mp, nV, dY, ec, M, aT, Lv, h, ey, wt, af, eu, TC, bf, s, cW, aW, mQ, hl, iy, kp, Z, sK, sE, ek, iq, kT, cT, ac, hc, JM, ei, lC); #c1(ALOX5AP) c2(NP_001620) c3(arachidonate 5-lipoxygenase-activating protein isoform 1) c4(119287) c5(119288, 119289, 119290, 119291, 119292) c6( ) c7(bM, dD, ea, e, aw, NT, cz, oR, bS, j, ct, v, eq, zP, TB, Mp, nV, dY, ec, M, aT, Lv, h, ey, wt, af, eu, TC, bf, s, cW, aW, mQ, hl, iy, kp, Z, sk, sE, ek, iq, KT, cT, ac, hc, JM, ei, lC); #c1(ALOXE3) c2(NP_001159432) c3(hydroperoxide isomerase ALOXE3 isoform 1 precursor) c4(63997, 63998) c5(63999, 64000, 64001, 64002, 64003, 64004, 64005, 64006, 64007, 64008, 64009, 64010, 64011) c6( ) c7(Ts, TD, fn, TE, m, ad); #c1(ALOXE3) c2(NP_067641) c3 (hydroperoxide isomerase ALOXE3 isoform 2) c4(209/70) c5(209171, 209172, 209/73, 209/74, 209175) c6( ) c7(Ts, TD, fn, TE, m, ad); #c1(ALP1) c2(NP_001622) c3(intestinal-type alkaline phosphatase precursor) c4(119293) c5(119294, 119295, 119296, 119297, 119298, 119299) c6( ) c7(T, if, eX, jh, fd, ci, qN, PJ, ai, ad, aE, dv, aT, kf, vH, eV, K, ae, cp, d, MD, nO, aW, Y, hl, aq, iV, iR, bw, bz, LB, ck, kP, iT, bE, HN, bt, B); #c1(ALPK1) c2(NP_001095876) c3(alpha-protein kinase 1 isoform 1) c4(40875) c5(40876, 40877, 40878, 40879, 40880, 40881, 40882, 40883, 40884, 40885, 40886, 40887, 40888, 40889, 40890, 40891) c6( ) c7(fU, nZ, iU, hG, ht, fe, aj, bS, v, ct, g, Z); #c1(ALPK1) c2(NP_001240813) c3(alpha-protein kinase 1 isoform 2) c4(88873) c5( ) c6( ) c7(fU, nZ, iU, hG, ht, fe, aj, bS, v, ct, g, Z); #c1(ALPK1) c2(XP_006714389) c3(alpha-protein kinase 1 isoform X2) c4(287819) c5(287820, 287821, 287822, 287823, 287824, 287825) c6( ) c7(fU, nZ, iU, hG, ht, fe, aj, bS, v, ct, g, Z); #c1(ALPK2) c2(NP_443179) c3(alpha-protein kinase 2) c4(229740) c5(229741, 229742, 229743, 229744, 229745, 229746) c6( ) c7(T, B, eM); #c1(ALPK3) c2(NP_065829) c3(alpha-protein kinase 3) c4(205713) c5(205714, 205715, 205716, 205717, 205718, 205719) c6( ) c7(TF, nC, nw, lR); #c1(ALPP) c2(NP_001623) c3(alkaline phosphatase, placental type preproprotein) c4(119300) c5(119301, 119302, 119303, 119304, 119305, 119306) c6( ) c7(T, wu, fd, bB, Tl, h, wm, nJ, bc, vT, HY, TH, eH, aq, eF, TJ, bE, TG, au, rx, y, B); #c1(ALPPL2) c2(NP_112603) c3(alkaline phosphatase, placental-like preproprotein) c4(221540) c5(221541, 221542, 221543, 221544, 221545, 221546) c6( ) c7(nJ, Bl, ac, lR, vT, bE, wu, ev, TK, bB, wm, an, on, nC); #c1(ALS2) c2(NP_001129217) c3(alsin isoform 2) c4(51217, 51218, 51219) c5(51220, 51221, 51222, 51223, 51224, 51225, 51226, 51227, 51228, 51229, 51230, 51231, 51232, 51233, 51234, 51235, 51236, 51237) c6( ) c7(c, lu, TL, TN, TP, aV, DI, TO, U, TR, aU, TM, K, PJ, xw, ln); #c1(ALS2) c2(NP_065970) c3(alsin isoform 1) c4(206506) c5(206507, 206508, 206509, 206510, 206511, 206512, 206513, 206514, 206515, 206516, 206517, 206518, 206519, 206520, 206521, 206522, 206523) c6( ) c7(c, lu, TL, TN, TP, aV, OI, TO, U, TQ, aU, TM, K, PJ, xw, Qn); #c1(ALS2) c2(XP_011509832) c3(alsin isoform X3) c4(300434, 300435) c5( ) c6( ) c7(c, lu, TL, TN, TP, aV, 0I, TO, U, TQ, aU, TM, K, PJ, xw, In); #c1(ALS2CL) c2(NP_001177636) c3(ALS2 C-terminal-like protein isoform 1) c4(72799) c5(72800, 72801, 72802, 72803, 72804, 72805, 72806, 72807, 72808, 72809) c6( ) c7(AW, c); #c1(ALS2CL) c2(NP_877576) c3(ALS2 C-terminal-like protein isoform 3) c4( ) c5(256912, 256913, 256914, 256915, 256916) c6( ) c7(AW, c); #c1(ALS2CL) c2(XP_005265082) c3(ALS2 C-terminal-like protein isoform X6) c4(279425) c5(279426) c6( ) c7(AW, c); #c1(ALS2CR12) c2(NP_001120863) c3(amyotrophic lateral sclerosis 2 chromosomal region candidate gene 12 protein isoform b) c4(44858) c5(44859, 44860, 44861, 44862, 44863, 44864) c6( ) c7(Y, dL, ht, ff, en); #c1(ALSZCR12) c2(NP_631902) c3(amyotrophic lateral sclerosis 2 chromosomal region candidate gene 12 protein isoform a) c4(238264) c5(238265, 238266, 238267, 238268, 238269, 238270, 238271, 238272, 238273, 238274) c6( ) c7(Y, dL, ht, ff, en); #c1(ALS2CR12) c2(XP_006712336) c3(amyotrophic lateral sclerosis 2 chromosomal region candidate gene 12 protein isoform X5) c4( ) c5(286597, 286598, 286599, 286600, 286601, 286602) c6( ) c7(Y, dL, ht, ff, en); #c1(ALS2CR12) c2(XP_011508908) c3(amyotrophic lateral sclerosis 2 chromosomal region candidate gene 12 protein isoform X1) c4(299483) c5( ) c6( ) c7(Y, dl, ht, ff, en); #c1(ALX1) c2(XP_005269222) c3(ALX homeobox protein 1 isoform X1) c4(282493) c5(282494, 282495, 282496, 282497, 282498, 282499, 282500) c6( ) c7(T, kj, lp, TU, TR, aW, Az, TS, aT, m, t, TT); #c1(ALX4) c2(NP_068745) c3 (homeobox protein aristaless-like 4) c4(210156) c5(210157, 210158, 210159, 210160, 210161, 210162) c6( ) c7(bL, T, TV, aw, bB, aj, TZ, j, q, jz, pN, h, pl, jf, TW, s, TY, Az, r, aq, g, bz, bE, gX, TX, zG); #c1(ALYREF) c2(NP_005773) c3(THO complex subunit 4) c4(159235, 159236) c5(159237, 159238, 159239, 159240, 159241, 159242, 159243, 159244, 159245, 159246, 159247, 159248) c6( ) c7(dA, c, ds, ff, h, en, cd); #c1(AMACR) c2(NP_001161067) c3(alpha-methylacyl-CoA racemase isoform 3) c4(65258) c5(65259, 65260, 65261, 65262, 65263, 65264) c6( ) c7(Ua, G, T, fM, Uc, LX, bO, aA, bB, n, v, Fb, ci, fr, rP, iJ, q, ad, fx, NS, aT, dL, AN, bV, ae, B, s, Ub, vT, aW, hl, LZ, r, fQ, fe, aF, g, cm, bz, Qx, Qi, es, ql, iA, bE, ln, nG, hj, mE, gZ, at, X); #c1(AMACR) c2(NP_055139) c3(alpha-methylacyl-CoA racemase isoform 1) c4(179242) c5(179243, 179244, 179245, 179246, 179247, 179248, 179249, 179250, 179251, 179252, 179253, 179254, 179255, 179256, 179257, 179258, 179259, 179260, 179261, 179262, 179263, 179264, 179265, 179266, 179267, 179268, 179269, 179270, 179271, 179272, 179273, 179274, 179275, 179276, 179277, 179278, 179279, 179280, 179281, 179282, 179283, 179284, 179285, 179286, 179287, 179288, 179289, 179290, 179291, 179292, 179293, 179294, 179295, 179296, 179297, 179298, 179299, 179300, 179301, 179302, 179303, 179304, 179305, 179306, 179307, 179308, 179309, 179310, 179311, 179312, 179313, 179314, 179315, 179316, 179317, 179318, 179319, 179320, 179321, 179322, 179323, 179324, 179325, 179326, 179327, 179328, 179329, 179330, 179331, 179332, 179333, 179334, 179335, 179336, 179337, 179338, 179339, 179340, 179341, 179342, 179343, 179344, 179345, 179346, 179347, 179348, 179349, 179350, 179351, 179352, 179353, 179354, 179355, 179356, 179357, 179358, 179359, 179360, 179361, 179362, 179363, 179364, 179365, 179366, 179367, 179368, 179369, 179370, 179371, 179372, 179373, 179374, 179375, 179376, 179377, 179378, 179379, 179380, 179381, 179382, 179383, 179384, 179385, 179386, 179387) c6(179388) c7(Ua, G, T, fM, Uc, LX, bO, aA, bB, n, v, Fb, ci, fr, rP, iJ, q, ad, fx, NS, aT, dL, AN, bV, ae, B, s, Ub, vT, aW, hl, LZ, r, fQ, fe, aF, g, cm, bz, Qx, Qi, es, ql, iA, bE, ln, nG, hj, mE, gZ, at, X); #c1(AMACR) c2(NP_976316) c3(alpha-methylacyl-CoA racemase isoform 2) c4(263128, 263129) c5(263130, 263131, 263132, 263133, 263134, 263135, 263136, 263137, 263138, 263139, 263140, 263141) c6( ) c7(Ua, G, T, fM, Uc, LX, bO, aA, bB, n, v, Fb, ci, fr, rP, iJ, q, ad, fx, NS, aT, dL, AN, bV, ae, B, s, Ub, vT, aW, hl, LZ, r, fQ, fe, aF, g, cm, bz, [x, Qi, es, ql, iA, bE, ln, nG, hj, mE, gZ, at, X); #c1(AMBN) c2(NP_057603) c3(ameloblastin precursor) c4(191586, 191587, 191588) c5(191589, 191590, 191591, 191592, 191593, 191594, 191595, 191596, 191597, 191598, 191599, 191600, 191601, 191602, 191603, 191604, 191605, 191606, 191607, 191608, 191609, 191610, 191611, 191612, 191613, 191614, 191615, 191616, 191617, 191618, 191619, 191620, 191621, 191622, 191623, 191624, 191625, 191626, 191627, 191628, 191629, 191630, 191631, 191632, 191633, 191634, 191635, 191636, 191637, 191638, 191639, 191640, 191641, 191642, 191643, 191644, 191645, 191646, 191647, 191648, 191649, 191650, 191651, 191652, 191653, 191654, 191655, 191656, 191657, 191658, 191659, 191660, 191661, 191662, 191663, 191664, 191665, 191666, 191667, 191668, 191669, 191670, 191671, 191672, 191673, 191674, 191675, 191676, 191677, 191678, 191679, 191680, 191681, 191682, 191683, 191684, 191685, 191686, 191687, 191688, 191689, 191690, 191691, 191692, 191693, 191694, 191695, 191696, 191697, 191698, 191699) c6(191700, 191701, 191702, 191703, 191704, 191705, 191706) c7(ki, Ud); #c1(AMBP) c2(NP_001624) c3(protein AMBP preproprotein) c4(119307, 119308) c5(119309, 119310, 119311, 119312, 119313, 119314, 119315, 119316, 119317, 119318, 119319, 119320) c6( ) c7(T, dD, id, qb, ea, e, rf, cz, fd, n, eq, ci, fr, eA, ad, wG, yt, Uh, aT, Uf, aW, Ue, ls, uF, nN, t, fq, dr, Ta, Ui, dB, Nk, Ug, wq, E, m, EY, Tc); #c1(AMBRA1) c2(NP_001254711) c3(activating molecule in BECN1-regulated autophagy protein 1 isoform 1) c4(94805, 94806, 94807, 94808, 94809, 94810, 94811, 94812, 94813, 94814, 94815, 94816, 94817, 94818, 94819, 94820, 94821, 94822, 94823) c5(94824, 94825, 94826, 94827, 94828, 94829, 94830, 94831, 94832, 94833, 94834, 94835, 94836, 94837, 94838, 94839, 94840, 94841, 94842, 94843, 94844, 94845, 94846, 94847, 94848, 94849, 94850, 94851, 94852, 94853, 94854, 94855, 94856, 94857, 94858, 94859, 94860, 94861, 94862, 94863, 94864, 94865, 94866, 94867, 94868, 94869, 94870, 94871, 94872, 94873, 94874, 94875, 94876, 94877, 94878, 94879, 94880, 94881, 94882) c6( ) c7(q, r); #c1(AMBRA1) c2(NP_001254712) c3(activating molecule in BECN1-regulated autophagy protein 1 isoform 3) c4(94883) c5(94884, 94885, 94886, 94887, 94888, 94889) c6( ) c7(q, r); #c1(AMBRA1) c2(NP_001287660) c3(activating molecule in BECN1-regulated autophagy protein 1 isoform 4) c4(112466) c5(112467, 112468, 112469, 112470, 112471, 112472, 112473, 112474, 112475, 112476) c6( ) c7(q, r); #c1(AMBRA1) c2(XP_005253068) c3(activating molecule in BECN1-regulated autophagy protein 1 isoform X3) c4(272115) c5(272116, 272117, 272118, 272119, 272120, 272121, 272122, 272123, 272124, 272125) c6( ) c7(q, r); #c1(AMBRA1) c2(XP_005253071) c3(activating molecule in BECN1-regulated autophagy protein 1 isoform X1) c4(272126) c5(272127, 272128, 272129, 272130, 272131, 272132) c6( ) c7(q, r); #c1(AMBRA1) c2(XP_006718322) c3(activating molecule in BECN1-regulated autophagy protein 1 isoform X2) c4(291705) c5(29/706, 291707, 29/708, 29/709, 29/710, 29/711) c6( ) c7(q, r); #c1(AMBRA1) c2(XP_011518516) c3(activating molecule in BECN1-regulated autophagy protein 1 isoform X5) c4(312629) c5( ) c6( ) c7(q, r); #c1(AMBRA1) c2(XP_011518517) c3(activating molecule in BECN1-regulated autophagy protein 1 isoform X6) c4( ) c5(312630) c6( ) c7(q, r); #c1(AMD1) c2(NP_001274143) c3(S-adenosylmethionine decarboxylase proenzyme isoform 3) c4(106062) c5(106063, 106064, 106065, 106066, 106067, 106068) c6( ) c7(T, qQ, v, ad, Ul, aT, bk, K, vd, Um, ae, cY, li, lR, aW, r, Un, Uk, W, Uj, lp, nw, lC); #c1(AMD1) c2(NP_001274144) c3(S-adenosylmethionine decarboxylase proenzyme isoform 4) c4( ) c5(106069, 106070, 106071, 106072, 106073, 106074, 106075, 106076, 106077, 106078, 106079) c6( ) c7(T, qQ, v, ad, Ul, aT, bK, K, vd, Um, ae, cY, li, lR, aW, r, Un, Uk, W, Uj, lp, nw, lC); #c1(AMD1) c2(NP_001274145) c3(S-adenosylmethionine decarboxylase proenzyme isoform 5) c4(106080) c5(106081, 106082, 106083, 106084, 106085, 106086) c6( ) c7(T, qQ, v, ad, Ul, aT, bk, K, vd, Um, ae, cY, li, lR, aW, r, Un, Uk, W, Uj, lp, nw, lC); #c1(AMD1) c2(NP_001625) c3(S-adenosylmethionine decarboxylase proenzyme isoform 1) c4(119321, 119322, 119323) c5(119324, 119325, 119326, 119327, 119328, 119329, 119330) c6( ) c7(T, qQ, v, ad, Ul, aT, bK, K, vd, Um, ae, cY, li, lR, aW, r, Un, Uk, W, Uj, lp, nw, lC); #c1(AMELX) c2(NP_001133) c3(amelogenin, X isoform isoform 1 precursor) c4(52814) c5(52815, 52816, 52817, 52818, 52819, 52820, 52821, 52822) c6(52823, 52824) c7(Uo, ka, T, dA, ec, pN, aW, ki, aT, bh, Ud, bt, ae, ai, ad, bc); #c1(AMELX) c2(NP_872622) c3 (amelogenin, X isoform isoform 2 precursor) c4(256539) c5(256540, 256541, 256542, 256543, 256544, 256545, 256546, 256547, 256548) c6(256549, 256550) c7(Uo, ka, T, dA, ec, pN, aW, ki, aT, bh, Ud, bt, ae, ai, ad, bc); #c1(AMELX) c2(XP_011543791) c3(amelogenin, X isoform isoform X1) c4(342897) c5(342898, 342899, 342900, 342901, 342902, 342903, 342904, 342905) c6(342906, 342907) c7(Uo, ka, T, dA, ec, pN, aW, ki, aT, bh, Ud, bt, ae, ai, ad, bc); #c1(AMELY) c2(NP_001134) c3(amelogenin, Y isoform precursor) c4(52825) c5(52826, 52827, 52828, 52829, 52830, 52831) c6( ) c7(Uo, B); #c1(AMELY) c2(XP_011529774) c3(amelogenin, Y isoform isoform X1) c4(325463, 325464) c5(325465, 325466, 325467, 325468, 325469, 325470, 325471, 325472, 325473, 325474, 325475, 325476, 325477, 325478, 325479, 325480, 325481, 325482, 325483, 325484, 325485, 325486, 325487, 325488, 325489, 325490, 325491, 325492, 325493, 325494, 325495, 325496, 325497, 325498, 325499, 325500, 325501, 325502, 325503, 325504, 325505, 325506, 325507, 325508, 325509, 325510, 325511, 325512, 325513, 325514, 325515, 325516, 325517, 325518, 325519, 325520, 325521, 325522, 325523, 325524, 325525, 325526, 325527, 325528, 325529, 325530, 325531, 325532, 325533, 325534, 325535, 325536, 325537, 325538, 325539, 325540, 325541, 325542, 325543, 325544, 325545, 325546, 325547, 325548, 325549, 325550, 325551, 325552, 325553, 325554, 325555, 325556, 325557, 325558, 325559, 325560, 325561, 325562, 325563) c6(325564) c7(Uo, B); #c1(AMER1) c2(NP_689637) c3(APC membrane recruitment protein 1) c4(243376, 243377) c5(243378, 243379, 243380, 243381, 243382, 243383, 243384, 243385, 243386, 243387, 243388, 243389, 243390, 243391, 243392) c6( ) c7(bF, es, T, aC, B, iO, Rq, Or, iA, ml, r, Up, nA, bt, q, er); #c1(AMER2) c2(NP_689917) c3(APC membrane recruitment protein 2 isoform 1) c4(244357) c5(244358, 244359, 244360, 244361, 244362, 244363) c6( ) c7(bF, er); #c1(AMER2) c2(XP_006719833) c3(APC membrane recruitment protein 2 isoform X2) c4(293019) c5(293020, 293021, 293022, 293023, 293024, 293025, 293026, 293027, 293028, 293029) c6( ) c7(bF, er); #c1(AMER3) c2(NP_001098663) c3(APC membrane recruitment protein 3) c4(41388, 41389, 41390) c5(41391, 41392, 41393, 41394, 41395, 41396, 41397, 41398, 41399, 41400, 41401, 41402, 41403, 41404, 41405, 41406, 41407, 41408, 41409, 41410, 41411, 41412) c6( ) c7(bF, q, r); #c1(AMFR) c2(NP_001135) c3(E3 ubiquitin-protein ligase AMFR) c4(53966) c5( ) c6( ) c7(av, T, eG, af, Y, Uq, dL, K, g, bz, B); #c1(AMFR) c2(XP_005255946) c3(E3 ubiquitin-protein ligase AMFR isoform X1) c4(273902) c5(273903, 273904, 273905, 273906, 273907, 273908) c6( ) c7(av, T, eG, af, Y, Uq, dL, K, g, bz, B); #c1(AMH) c2(NP_000470) c3(muellerian-inhibiting factor precursor) c4(9417) c5(9418, 9419, 9420, 9421, 9422, 9423) c6( ) c7(T, Ut, UH, ea, if, cL, bh, Ul, ip, gJ, il, Pl, dV, K, hH, eL, bc, Us, Ur, hm, UC, yo, aW, az, HA, UJ, pD, cm, eM, U, cj, QF, uJ, m, Ux, eg, Uz, Uw, UA, dD, aw, aj, nA, qN, yn, PD, UD, dL, yi, A, fe, Pp, dB, Uv, at, eo, Pr, bL, pF, pl, aA, fF, v, j, C, Ns, V, o, J, UE, er, lg, BJ, aT, cy, ae, kl, s, vT, Y, bu, t, pb, ua, ac, fD, UF, aZ, dM, aD, jh, eq, ah, Uy, ad, qC, dP, vJ, UG, hD, ji, ul, d, cr, eZ, aF, hp, Fq, iV, js, Uu, UB, be, lf, y); #c1(AMHR2) c2(NP_001158162) c3(anti-Muellerian hormone type-2 receptor isoform 2 precursor) c4(63225) c5(63226, 63227, 63228, 63229, 63230, 63231) c6( ) c7(Le, T, pY, aZ, if, nA, qN, ad, dP, hW, vJ, il, BJ, aT, PD, UK, Ur, yi, kl, UL, aW, UJ, g, t, bE, UB, Uv, m, Th); #c1(AMHR2) c2(NP_001158163) c3(anti-Muellerian hormone type-2 receptor isoform 3 precursor) c4(63232) c5(63233, 63234, 63235, 63236, 63237, 63238) c6( ) c7(Le, T, pY, aZ, if, nA, qN, ad, dP, hW, vJ, il, BJ, aT, PD, UK, Ur, yi, kl, UL, aW, UJ, g, t, bE, UB, Uv, m, Th); #c1(AMHR2) c2(NP_065434) c3(anti-Muellerian hormone type-2 receptor isoform 1 precursor) c4(204784) c5(204785, 204786, 204787, 204788, 204789, 204790) c6( ) c7(Le, T, pY, aZ, if, nA, qN, ad, dP, hW, vJ, il, BJ, aT, PD, UK, Ur, yi, kl, UL, aW, UJ, g, t, bE, UB, Uv, m, Th); #c1(AMHR2) c2(XP_011536483) c3(anti-Muellerian hormone type-2 receptor isoform X9) c4( ) c5(333829) c6( ) c7(Le, T, pY, aZ, if, nA, qN, ad, dP, hW, vJ, il, BJ, aT, PD, UK, Ur, yi, kl, UL, aW, UJ, g, t, bE, UB, Uv, m, Th); #c1(AMICA1) c2(NP_001091996) c3(junctional adhesion molecule-like isoform 1 precursor) c4(39215) c5(39216, 39217, 39218, 39219, 39220, 39221) c6( ) c7(IT); #c1(AMICA1) c2(NP_694938) c3(junctional adhesion molecule-like isoform 2) c4(245427) c5(245428, 245429, 245430, 245431, 245432, 245433) c6( ) c7(IT); #c1(AMICA1) c2(XP_005271464) c3(junctional adhesion molecule-like isoform X2) c4(283429) c5( ) c6( ) c7(IT); #c1(AMICA1) c2(XP_011540909) c3 (junctional adhesion molecule-like isoform X3) c4(339451) c5(339452, 339453, 339454, 339455, 339456, 339457, 339458, 339459, 339460, 339461, 339462, 339463) c6( ) c7(IT); #c1(AMIGO2) c2(NP_862830) c3(amphoterin-induced protein 2 precursor) c4(255811) c5(255812, 255813, 255814, 255815, 255816, 255817, 255818, 255819, 255820, 255821, 255822, 255823, 255824, 255825, 255826, 255827, 255828, 255829) c6( ) c7(JI, af, g, an); #c1(AMMECR1) c2(NP_001020751) c3(AMME syndrome candidate gene 1 protein isoform 2) c4(28989) c5(28990, 28991, 28992, 28993, 28994, 28995, 28996) c6( ) c7(UM); #c1(AMMECR1) c2(NP_001165160) c3(AMME syndrome candidate gene 1 protein isoform 3) c4( ) c5(67523, 67524, 67525, 67526, 67527) c6( ) c7(UM); #c1(AMMECR1) c2(NP_056180) c3(AMME syndrome candidate gene 1 protein isoform 1) c4(186169, 186170) c5(186171, 186172, 186173, 186174, 186175, 186176, 186177) c6( ) c7(UM); #c1(AMN) c2(NP_112205) c3(protein amnionless precursor) c4(220782) c5(220783, 220784, 220785, 220786, 220787, 220788) c6( ) c7(ck, x, UO, UP, iU, mD, mA, E, mz, my, UN, dF, ao, mH); #c1(AMOT) c2(XP_005262147) c3(angiomotin isoform X2) c4(277754, 277755) c5(277756, 277757, 277758, 277759, 277760, 277761, 277762, 277763, 277764, 277765, 277766) c6( ) c7(T, d, py, aW, aT, GQ, aj, ci, fq, af); #c1(AMOT) c2(XP_011529177) c3 (angiomotin isoform X1) c4(324553, 324554, 324555) c5(324556, 324557, 324558, 324559, 324560, 324561, 324562, 324563, 324564, 324565, 324566, 324567, 324568, 324569, 324570, 324571, 324572, 324573, 324574, 324575, 324576, 324577, 324578, 324579) c6( ) c7(T, d, py, aW, aT, GQ, aj, ci, fq, af); #c1(AMOTL1) c2(NP_001287936) c3(angiomotin-like protein 1 isoform 2) c4(113240) c5(113241, 113242, 113243, 113244, 113245, 113246) c6( ) c7(aT); #c1(AMOTL1) c2(NP_570899) c3(angiomotin-like protein 1 isoform 1) c4(234372, 234373, 234374) c5(234375, 234376, 234377, 234378, 234379, 234380, 234381, 234382, 234383, 234384, 234385, 234386, 234387, 234388, 234389, 234390, 234391, 234392) c6( ) c7(aT); #c1(AMPD2) c2(XP_011539550) c3(AMP deaminase 2 isoform X3) c4( ) c5(337758) c6( ) c7(dD, UQ, fc, U, UR, eq, C); #c1(AMPD3) c2(NP_000471) c3(AMP deaminase 3 isoform (A) c4(9424, 9425, 9426, 9427) c5(9428, 9429, 9430, 9431, 9432, 9433, 9434) c6( ) c7(UT, US, X, K); #c1(AMPD3) c2(NP_001020560) c3(AMP deaminase 3 isoform 1B) c4(28920) c5(28921, 28922, 28923, 28924, 28925, 28926, 28927, 28928, 28929, 28930) c6( ) c7(UT, US, X, K); #c1(AMPD3) c2(NP_001020561) c3(AMP deaminase 3 isoform (C) c4(28931) c5(28932, 28933, 28934, 28935, 28936, 28937, 28938, 28939, 28940, 28941, 28942, 28943, 28944, 28945, 28946, 28947, 28948, 28949, 28950, 28951, 28952, 28953, 28954) c6( ) c7(UT, US, X, K); #c1(AMPH) c2(NP_001626) c3(amphiphysin isoform 1) c4(119331) c5(119332, 119333, 119334, 119335, 119336, 119337) c6( ) c7(IF, T, UW, av, aW, UU, xz, oj, aT, aA, bK, UX, f, fb, UV); #c1(AMPH) c2(NP_647477) c3(amphiphysin isoform 2) c4(238649, 238650) c5(238651, 238652, 238653, 238654, 238655, 238656, 238657, 238658, 238659, 238660, 238661, 238662) c6( ) c7(IF, T, UW, av, aW, UU, xz, oj, aT, aA, bK, UX, f, fb, UV); #c1(AMPH) c2(XP_006715752) c3(amphiphysin isoform X1) c4(289757) c5( ) c6( ) c7(IF, T, UW, av, aW, UU, xz, oj, aT, aA, bK, UX, f, fb, UV); #c1(AMPH) c2(XP_006715753) c3(amphiphysin isoform X3) c4(289758, 289759) c5(289760, 289761, 289762, 289763, 289764, 289765, 289766, 289767, 289768, 289769, 289770, 289771) c6( ) c7(IF, T, UW, av, aW, UU, xz, oj, aT, aA, bK, UX, f, fb, UV); #c1(AMPH) c2(XP_006715754) c3(amphiphysin isoform X4) c4( ) c5(289772, 289773, 289774, 289775, 289776, 289777) c6( ) c7(IF, T, UW, av, aW, UU, xz, oj, aT, aA, bK, UX, f, fb, UV); #c1(AMT) c2(NP_000472) c3 (aminomethyltransferase, mitochondrial isoform 1 precursor) c4(9435) c5(9436, 9437, 9438, 9439, 9440, 9441, 9442) c6( ) c7(ar, hl, UY, lp, ha, jh, vG, at, y, UZ); #c1(AMT) c2(NP_001158182) c3(aminomethyltransferase, mitochondrial isoform 2 precursor) c4(63267) c5(63268, 63269, 63270, 63271, 63272, 63273) c6( ) c7(ar, hl, UY, lp, ha, jh, vG, at, y, UZ); #c1(AMT) c2(NP_001158183) c3(aminomethyltransferase, mitochondrial isoform 3 precursor) c4(63274, 63275) c5(63276, 63277, 63278, 63279, 63280, 63281, 63282, 63283, 63284, 63285, 63286) c6( ) c7(ar, hl, UY, lp, ha, jh, vG, at, y, UZ); #c1(AMT) c2(NP_001158184) c3(aminomethyltransferase, mitochondrial isoform 4 precursor) c4(63287, 63288) c5(63289, 63290, 63291, 63292, 63293, 63294, 63295, 63296, 63297, 63298, 63299, 63300) c6( ) c7(ar, hl, UY, lp, ha, jh, vG, at, y, UZ); #c1(AMZ1) c2(XP_011513452) c3(archaemetzincin-1 isoform X1) c4(305704) c5(305705, 305706, 305707, 305708, 305709, 305710) c6( ) c7(Va); #c1(ANAPC1) c2(NP_073153) c3(anaphase-promoting complex subunit 1) c4(212829) c5(212830, 212831, 212832, 212833, 212834, 212835) c6( ) c7(X, Vb); #c1(ANAPC1) c2(XP_011509939) c3(anaphase-promoting complex subunit 1 isoform X5) c4( ) c5(300544) c6( ) c7(X, Vb); #c1(ANAPC10) c2(NP_001243638) c3 (anaphase-promoting complex subunit 10 isoform 2) c4( ) c5(91273, 91274, 91275, 91276) c6( ) c7(T, s, r, m, t, q); #c1(ANAPC10) c2(NP_001243641) c3(anaphase-promoting complex subunit 10 isoform 4) c4( ) c5(91277, 91278, 91279, 91280, 91281) c6( ) c7(T, s, r, m, t, q); #c1(ANAPC10) c2(NP_055700) c3(anaphase-promoting complex subunit 10 isoform 1) c4( ) c5(182861, 182862, 182863, 182864, 182865, 182866, 182867, 182868, 182869, 182870, 182871, 182872, 182873, 182874, 182875, 182876, 182877, 182878, 182879) c6( ) c7(T, s, r, m, t, q); #c1(ANAPC10) c2(XP_011529823) c3(anaphase-promoting complex subunit 10 isoform X3) c4( ) c5(325629, 325630) c6( ) c7(T, s, r, m, t, q); #c1(ANAPC10) c2(XP_011529834) c3(anaphase-promoting complex subunit 10 isoform X12) c4( ) c5(325631, 325632, 325633, 325634, 325635) c6( ) c7(T, s, r, m, t, q); #c1(ANAPC1I) c2(NP_001002244) c3(anaphase-promoting complex subunit 11 isoform 1) c4(19441) c5(19442, 19443, 19444, 19445, 19446, 19447) c6( ) c7(iR, aq, T, bt); #c1(ANAPC1I) c2(NP_001276346) c3(anaphase-promoting complex subunit 11 isoform 2) c4(108203) c5(108204, 108205, 108206, 108207, 108208, 108209, 108210, 108211, 108212, 108213) c6( ) c7(iR, aq, T, bt); #c1(ANAPC1I) c2(NP_001276349) c3(anaphase-promoting complex subunit II isoform 4) c4( ) c5(108214) c6( ) c7(iR, aq, T, bt); #c1(ANAPC13) c2(NP_001229304) c3(anaphase-promoting complex subunit 13) c4(84/72) c5(84173, 84174, 84175, 84176, 84177, 84178, 84179, 84180, 84181, 84182, 84183, 84184, 84185, 84186, 84187, 84188, 84189, 84190) c6( ) c7(xb); #c1(ANAPC2) c2(NP_037498) c3(anaphase-promoting complex subunit 2) c4(175859) c5(175860, 175861, 175862, 175863, 175864, 175865) c6( ) c7(bF, T, K, m, g, t, iu); #c1(ANAPC4) c2(NP_001273685) c3(anaphase-promoting complex subunit 4 isoform 1) c4(105615, 105616) c5(105617, 105618, 105619, 105620, 105621, 105622) c6( ) c7(aW, y, aT); #c1(ANAPC4) c2(NP_037499) c3 (anaphase-promoting complex subunit 4 isoform 2) c4(175866) c5(175867, 175868, 175869, 175870, 175871, 175872) c6( ) c7(aW, y, aT); #c1(ANAPC4) c2(XP_011512141) c3(anaphase-promoting complex subunit 4 isoform X2) c4( ) c5(302733, 302734, 302735, 302736, 302737, 302738) c6( ) c7(aW, y, aT); #c1(ANAPC5) c2(NP_001131031) c3(anaphase-promoting complex subunit 5 isoform b) c4(52545) c5(52546, 52547, 52548, 52549, 52550, 52551, 52552, 52553, 52554, 52555, 52556) c6( ) c7(K); #c1(ANAPC5) c2(NP_057321) c3(anaphase-promoting complex subunit 5 isoform a) c4(190096, 190097, 190098, 190099) c5(190100, 190101, 190102, 190103, 190104, 190105, 190106) c6( ) c7(K); #c1(ANAPC5) c2(XP_005253957) c3(anaphase-promoting complex subunit 5 isoform X1) c4(272772) c5(272773, 272774, 272775, 272776, 272777, 272778, 272779, 272780, 272781, 272782, 272783, 272784, 272785, 272786, 272787, 272788, 272789) c6( ) c7(K); #c1(ANAPC5) c2(XP_006719512) c3(anaphase-promoting complex subunit 5 isoform X2) c4( ) c5(292627, 292628, 292629, 292630, 292631) c6( ) c7(K); #c1(ANAPC7) c2(NP_001131136) c3(anaphase-promoting complex subunit 7 isoform b) c4(52599) c5(52600, 52601, 52602, 52603, 52604, 52605) c6( ) c7(aW, AD, Dz, aT); #c1(ANAPC7) c2(NP_057322) c3(anaphase-promoting complex subunit 7 isoform a) c4(190107) c5(190108, 190109, 190110, 190/11, 190112, 190113) c6( ) c7(aW, AD, Dz, aT); #c1(ANG) c2(NP_001091046) c3(angiogenin precursor) c4(38359) c5(38360, 38361, 38362, 38363, 38364, 38365, 38366, 38367, 38368, 38369) c6( ) c7(sY, Ve, T, eR, DI, iW, fd, jh, aA, cz, n, bB, Fb, Vd, ci, fr, PJ, nE, aY, Vh, q, ad, ai, ip, Jm, dP, av, iu, no, aE, Vf, aT, lG, Jn, Vc, K, an, nJ, ae, eG, Di, JI, aW, Y, ev, Vg, r, TO, aq, Qm, g, cl, cm, uC, Vi, bw, ac, c, zr, bE, U, cj, m, y, bt, Vj, B); #c1 (ANGPT1) c2(NP_001137) c3(angiopoietin-1 isoform 1 precursor) c4(56038, 56039, 56040, 56041) c5( ) c6( ) c7(ID, bM, T, aC, ea, if, al, eb, bB, fd, ci, Vp, xE, aE, lv, fm, pX, M, dV, K, hu, hm, ly, aW, Ls, hl, g, as, bw, Vm, y, eM, iT, uJ, m, dD, Vo, aw, ht, n, aj, fr, qN, cd, ai, Vs, Bl, gd, Jn, cY, wC, A, fQ, aq, op, lx, dB, lf, va, B, tq, jv, fM, fG, yk, jx, aA, cz, fF, v, j, C, q, o, J, eA, eJ, hb, Vr, jZ, aT, cy, bq, ae, dl, nO, oV, r, t, Vk, Vq, Vn, ck, ac, wO, dh, dy, fD, bt, gx, kh, eq, hF, ad, Jm, dP, av, py, yt, iY, uD, fb, d, lR, xs, cr, aF, VI, Fq, bz, ek, gk, bE, ll, aS, bC, ei, lC, iu, us); #c1(ANGPT1) c2(NP_001186788) c3(angiopoietin-1 isoform 2 precursor) c4(78580, 78581, 78582) c5(78583, 78584, 78585, 78586, 78587, 78588, 78589, 78590, 78591, 78592, 78593) c6(78594, 78595) c7(ID, bM, T, aC, ea, if, al, eb, bB, fd, ci, Vp, xE, aE, lv, fm, pX, M, dV, K, hu, hm, ly, aW, Ls, hl, g, as, bw, Vm, y, eM, iT, uJ, m, dD, Vo, aw, ht, n, aj, fr, qN, cd, ai, Vs, Bl, gd, Jn, cY, wC, A, fQ, aq, op, lx, dB, lf, va, B, tq, jv, fM, fG, yk, jx, aA, cz, fF, v, j, C, q, o, J, eA, eJ, hb, Vr, jZ, aT, cy, bq, ae, dl, nO, oV, r, t, Vk, Vq, Vn, ck, ac, wO, dh, dy, fD, bt, gx, kh, eq, hF, ad, Jm, dP, av, py, yt, iY, uD, fb, d, lR, xs, cr, aF, VI, Fq, bz, ek, gk, bE, ll, aS, bC, ei, lC, iu, us); #c1(ANGPT2) c2(NP_001112359) c3(angiopoietin-2 isoform b precursor) c4(43312) c5(43313, 43314, 43315, 43316, 43317, 43318) c6( ) c7(ID, T, aC, UH, ea, e, fd, ci, bv, xE, aE, lv, Bc, pX, M, dV, Vv, K, eL, tw, ly, aW, Ls, lm, g, as, cm, uC, bw, xK, eM, nB, AM, vA, mx, vy, cj, Hb, m, eg, fy, dD, vl, sw, aw, n, aj, ww, Mi, fr, eh, cd, br, EI, ai, Vs, ft, jz, do, wG, gd, h, dl, ey, bl, cY, A, fQ, Vt, aq, kp, yq, mo, cM, op, lx, hq, Vu, Vy, lf, bm, at, va, bD, B, fM, jx, aA, cR, tc, cz, fF, j, v, C, wo, AD, q, J, eA, eJ, mj, rS, aT, cy, bq, ae, dl, eu, fO, r, cl, t, Vn, ov, ck, wg, kP, ac, nM, wO, DW, dh, dy, bt, gx, sY, kh, uG, Vz, dG, eq, jE, tK, ad, wD, hX, Jm, dP, av, py, Cj, sN, vw, fb, d, lR, tb, Vx, cr, aF, Fq, bz, qa, Hp, bE, ll, Vw, ei, lC, vt, y); #c1(ANGPT2) c2(NP_001112360) c3(angiopoietin-2 isoform c precursor) c4(43319) c5(43320, 43321, 43322, 43323, 43324, 43325) c6( ) c7(ID, T, aC, UH, ea, e, fd, ci, bv, xE, aE, lv, Bc, pX, M, dV, Vv, K, eL, tw, ly, aW, Ls, Um, g, as, cm, uC, bw, xK, eM, nB, AM, vA, mx, vy, cj, Hb, m, eg, fy, dD, vl, sw, aw, n, aj, ww, Mi, fr, eh, cd, br, EI, ai, Vs, ft, jz, do, wG, gd, h, dL, ey, bl, cY, A, fQ, Vt, aq, kp, yq, mo, cM, op, lx, hq, Vu, Vy, lf, bm, at, va, bD, B, fM, jx, aA, cR, tc, cz, fF, j, v, C, wo, AD, q, J, eA, eJ, mj, rS, aT, cy, bq, ae, dl, eu, fO, r, cl, t, Vn, ov, ck, wg, kP, ac, nM, wJ, DW, dh, dy, bt, gx, sY, kh, uG, Vz, dG, eq, jE, tK, ad, wD, hX, Jm, dP, av, py, Cj, sN, vw, fb, d, lR, tb, Vx, cr, aF, Fq, bz, qa, Hp, bE, ll, Vw, ei, lC, vt, y); #c1(ANGPT2) c2(NP_001138) c3(angiopoietin-2 isoform a precursor) c4(58597) c5( ) c6( ) c7(ID, T, aC, UH, ea, e, fd, ci, bv, xE, aE, lv, Bc, pX, M, dV, Vv, K, eL, tw, ly, aW, Ls, Am, g, as, cm, uC, bw, xK, eM, nB, AM, vA, mx, vy, cj, Hb, m, eg, fy, dD, vl, sw, aw, n, aj, ww, Mi, fr, eh, cd, br, EI, ai, Vs, ft, jz, do, wG, gd, h, dL, ey, bl, cY, A, fQ, Vt, aq, kp, yq, mo, cM, op, lx, hq, Vu, Vy, lf, bm, at, va, bD, B, fM, jx, aA, cR, tc, cz, fF, j, v, C, wo, AO, q, J, eA, eJ, mj, rS, aT, cy, bq, ae, dl, eu, f, r, cl, t, Vn, ov, ck, wg, kP, ac, nM, wO, DW, dh, dy, bt, gx, sY, kh, uG, Vz, dG, eq, jE, tK, ad, wD, hX, Jm, dP, av, py, Cj, sN, vw, fb, d, lR, tb, Vx, cr, af, Fq, bz, qa, Hp, bE, ll, Vw, ei, lC, vt, y); #c1(ANGPT4) c2(NP_057069) c3(angiopoietin-4 precursor) c4(188761) c5(188762, 188763, 188764, 188765, 188766, 188767) c6( ) c7(eG, d, BN, af, HI, hc, Ls, hy, yq, aS, ci, eA, fr, wA, fb, ne, op, EI); #c1(ANGPT4) c2(XP_005260777) c3(angiopoietin-4 isoform X3) c4(276871) c5(276872, 276873, 276874, 276875, 276876, 276877) c6( ) c7(eG, d, BN, af, HI, hc, Ls, hy, yq, aS, ci, eA, fr, wA, fb, ne, op, EI); #c1(ANGPTL1) c2(NP_004664) c3(angiopoietin-related protein 1 precursor) c4(148644, 148645, 148646) c5(148647, 148648, 148649, 148650, 148651, 148652, 148653, 148654, 148655, 148656, 148657, 148658, 148659, 148660, 148661, 148662, 148663, 148664, 148665, 148666, 148667, 148668, 148669, 148670, 148671, 148672, 148673, 148674, 148675, 148676) c6(148677, 148678, 148679, 148680, 148681, 148682, 148683, 148684) c7(T, BC, BD, fd, hy, ci, hF, eA, EI, av, aT, eG, B, BN, aW, Y, oV, aq, bz, ek, ck, ac, VA, ne); #c1(ANGPTL2) c2(NP_036230) c3 (angiopoietin-related protein 2 precursor) c4(172476, 172477) c5(172478, 172479, 172480, 172481, 172482, 172483, 172484, 172485, 172486, 172487, 172488, 172489, 172490, 172491, 172492, 172493) c6(172494, 172495, 172496, 172497) c7(T, dD, dA, ea, e, aA, cR, v, j, BF, eq, q, Pu, ad, Pv, aE, h, By, ae, Pw, eH, r, lo, BG, BE, eF, t, iQ, dh, bm, wR, m); #c1(ANGPTL4) c2(NP_001034756) c3(angiopoietin-related protein 4 isoform b precursor) c4(32256) c5(32257, 32258, 32259, 32260, 32261, 32262) c6( ) c7(bL, T, dD, aC, nz, sw, ea, e, aw, aA, cR, zN, v, j, ci, ah, bX, eq, fr, q, Pu, ai, eA, Pv, py, xE, RI, yt, nX, kl, aT, cy, jy, K, Pw, PK, NE, aW, ev, eH, fc, r, dq, aq, aF, yq, g, eF, t, iK, es, c, dh, mf, m, cP, ei, gT, B, VB); #c1(ANGPTL4) c2(NP_647475) c3 (angiopoietin-related protein 4 isoform a precursor) c4(238635, 238636) c5(238637, 238638, 238639, 238640, 238641, 238642, 238643, 238644, 238645, 238646, 238647, 238648) c6( ) c7(bL, T, dD, aC, nz, sw, ea, e, aw, aA, cR, zN, v, j, ci, ah, bX, eq, fr, q, Pu, ai, eA, Pv, py, xE, RI, yt, nX, kl, aT, cy, jy, K, Pw, PK, NE, aW, ev, eH, fc, r, dq, aq, af, yq, g, eF, t, iK, es, c, dh, mf, m, cP, ei, gT, B, VB); #c1(ANGPTL6) c2(NP_114123) c3 (angiopoietin-related protein 6 precursor) c4( ) c5(222253, 222254, 222255, 222256, 222257) c6( ) c7(LC, om, v, q, r); #c1(ANGPTL6) c2(XP_011526651) c3 (angiopoietin-related protein 6 isoform X3) c4(320960) c5(320961) c6( ) c7(LC, om, v, q, r); #c1(ANK1) c2(NP_000028) c3(ankyrin-1 isoform 3) c4(267, 268, 269) c5(270, 271, 272, 273, 274, 275, 276, 277, 278, 279, 280, 281) c6( ) c7(bL, bM, T, ai, sg, ad, Bc, rw, VI, aT, VD, af, VF, VC, mR, Fm, zD, vs, VE, t, VH, VJ, c, VG, m, lS, VK); #c1(ANK1) c2(NP_001135917) c3 (ankyrin-1 isoform 10 precursor) c4(53545) c5(53546, 53547, 53548, 53549, 53550, 53551) c6( ) c7(bL, bM, T, ai, sg, ad, Bc, rw, VI, aT, VD, af, VF, VC, mR, Fm, zD, vs, VE, t, VH, VJ, c, VG, m, lS, VK); #c1(ANK1) c2(NP_001135918) c3(ankyrin-1 isoform 9) c4(53552) c5(53553, 53554, 53555, 53556, 53557, 53558) c6( ) c7(bL, bM, T, ai, sg, ad, Bc, rw, VI, aT, VD, af, VF, VC, mR, Fm, zD, vs, VE, t, VH, VJ, c, VG, m, lS, VK); #c1(ANK1) c2(NP_065208) c3 (ankyrin-1 isoform 4) c4( ) c5(204595, 204596, 204597, 204598, 204599) c6( ) c7(bL, bM, T, ai, sg, ad, Bc, rw, VI, aT, VD, af, VF, VC, mR, Fm, zD, vs, VE, t, VH, VJ, c, VG, m, lS, VK); #c1(ANK1) c2(NP_065209) c3(ankyrin-1 isoform 1) c4(204600, 204601, 204602) c5(204603, 204604, 204605, 204606, 204607, 204608, 204609, 204610, 204611, 204612, 204613, 204614, 204615, 204616, 204617, 204618, 204619, 204620, 204621) c6( ) c7(bL, bM, T, ai, sg, ad, Bc, rw, VI, aT, VD, af, VF, VC, mR, Fm, zD, vs, VE, t, VH, VJ, c, VG, m, lS, VK); #c1(ANK1) c2(NP_065211) c3(ankyrin-1 isoform 5 precursor) c4(204622) c5(204623, 204624, 204625, 204626, 204627, 204628) c6( ) c7(bL, bM, T, ai, sg, ad, Bc, rw, VI, aT, VD, af, VF, VC, mR, Fm, zD, vs, VE, t, VH, VJ, c, VG, m, lS, VK); #c1(ANK1) c2(NP_065213) c3(ankyrin-1 isoform 7 precursor) c4(204629) c5(204630, 204631, 204632, 204633, 204634, 204635, 204636, 204637, 204638, 204639) c6( ) c7(bL, bM, T, ai, sg, ad, Bc, rw, VI, aT, VD, af, VF, VC, mR, Fm, zD, vs, VE, t, VH, VJ, c, VG, m, lS, VK); #c1(ANK1) c2(XP_011542799) c3(ankyrin-1 isoform X8) c4(341431) c5(341432, 341433, 341434, 341435, 341436, 341437) c6( ) c7(bL, bM, T, ai, sg, ad, Bc, rw, VI, aT, VD, af, VF, VC, mR, Fm, zD, vs, VE, t, VH, VJ, c, VG, m, lS, VK); #c1(ANK2) c2(NP_001120965) c3(ankyrin-2 isoform 3) c4(44996, 44997) c5(44998, 44999, 45000, 45001, 45002, 45003, 45004, 45005, 45006, 45007, 45008, 45009, 45010, 45011, 45012, 45013, 45014, 45015, 45016) c6( ) c7(VJ, aw, Bc, Fm, oe, VL, ha, HC, cV, gZ, e, cy, vs, j, pw, VM, cm, cd, HO, ad, mu); #c1(ANK2) c2(NP_001139) c3(ankyrin-2 isoform 1) c4( ) c5( ) c6( ) c7(VJ, aw, Bc, Fm, oe, VL, ha, HC, cV, gZ, e, cy, vs, j, pw, VM, cm, cd, HO, ad, mu); #c1(ANK2) c2(NP_066187) c3(ankyrin-2 isoform 2) c4(206810) c5(206811, 206812, 206813, 206814, 206815, 206816, 206817) c6( ) c7(VJ, aw, Bc, Fm, oe, VL, ha, HC, cV, gZ, e, cy, vs, j, pw, VM, cm, cd, HO, ad, mu); #c1(ANK2) c2(XP_005263002) c3(ankyrin-2 isoform X4) c4(278198) c5(278199, 278200, 278201, 278202, 278203, 278204) c6( ) c7(VJ, aw, Bc, Fm, oe, VL, ha, HC, cV, gZ, e, cy, vs, j, pw, VM, cm, cd, HO, ad, mu); #c1(ANK2) c2(XP_006714255) c3(ankyrin-2 isoform X9) c4(287664) c5(287665, 287666, 287667, 287668, 287669, 287670) c6( ) c7(VJ, aw, Bc, Fm, oe, VL, ha, HC, cV, gZ, e, cy, vs, j, pw, VM, cm, cd, HO, ad, mu); #c1(ANK3) c2(NP_001191332) c3(ankyrin-3 isoform 3) c4(81405) c5(81406, 81407, 81408, 81409, 81410, 81411) c6( ) c7(uk, bO, bS, uu, xg, aR, rC, bK, K, he, VN, VP, af, gl, vo, bN, hq, sk, VO, cA, Ei, uJ, lB, cB); #c1(ANK3) c2(NP_001191333) c3 (ankyrin-3 isoform 4) c4(81412) c5(81413, 81414, 81415, 81416, 81417, 81418) c6( ) c7(uk, bO, bS, uu, xg, aR, rC, bK, K, he, VN, VP, af, gl, vo, bN, hq, sk, VO, cA, Ei, uJ, lB, cB); #c1(ANK3) c2(NP_066267) c3(ankyrin-3 isoform 1) c4(206849, 206850, 206851, 206852, 206853) c5(206854, 206855, 206856, 206857, 206858, 206859) c6( ) c7(uk, bO, bS, uu, xg, aR, rC, bK, K, he, VN, VP, af, gl, vo, bN, hq, sk, VO, cA, Ei, uJ, lB, cB); #c1(ANK3) c2(XP_005269772) c3(ankyrin-3 isoform X23) c4(282787, 282788, 282789) c5(282790, 282791, 282792, 282793, 282794, 282795) c6( ) c7(uk, bO, bS, uu, xg, aR, rC, bK, K, he, VN, VP, af, gl, vo, bN, hq, sk, VO, cA, Ei, uJ, lB, cB); #c1(ANK3) c2(XP_005269773) c3(ankyrin-3 isoform X25) c4(282796) c5(282797, 282798, 282799, 282800, 282801, 282802) c6( ) c7(uk, bO, bS, uu, xg, aR, rC, bK, K, he, VN, VP, af, gl, vo, bN, hq, sk, VO, cA, Ei, uJ, lB, cB); #c1(ANK3) c2(XP_011538017) c3(ankyrin-3 isoform X20) c4( ) c5(335563, 335564, 335565, 335566, 335567, 335568) c6( ) c7(uk, bO, bS, uu, xg, aR, rC, bK, K, he, VN, VP, af, gl, vo, bN, hq, sk, VO, cA, Ei, uJ, lB, cB); #c1(ANK3) c2(XP_011538020) c3(ankyrin-3 isoform X26) c4( ) c5(335569, 335570, 335571, 335572, 335573, 335574, 335575, 335576, 335577, 335578, 335579, 335580, 335581, 335582, 335583, 335584, 335585, 335586) c6( ) c7(uk, bO, bS, uu, xg, aR, rC, bK, K, he, VN, VP, af, gl, vo, bN, hq, sk, VO, cA, Ei, uJ, lB, cB); #c1(ANKFN1) c2(NP_694960) c3(ankyrin repeat and fibronectin type-11l domain-containing protein 1) c4(245492) c5(245493, 245494, 245495, 245496, 245497, 245498) c6( ) c7(jb, V, Nc); #c1(ANKFN1) c2(XP_011522734) c3(ankyrin repeat and fibronectin type-III domain-containing protein 1 isoform X7) c4( ) c5(317267) c6( ) c7(jb, V, Nc); #c1(ANKFY1) c2(NP_057460) c3(rabankyrin-5 isoform 1) c4(191036) c5(191037, 191038, 191039, 191040, 191041, 191042, 191043, 191044, 191045, 191046) c6( ) c7(eR); #c1(ANKFY1) c2(XP_005256736) c3(rabankyrin-5 isoform X3) c4(274305) c5( ) c6( ) c7(eR); #c1(ANKFY1) c2(XP_011522233) c3(rabankyrin-5 isoform X5) c4(316856) c5(316857, 316858, 316859, 316860, 316861, 316862) c6( ) c7(eR); #c1(ANKFY1) c2(XP_011522234) c3(rabankyrin-5 isoform X6) c4( ) c5(316863, 316864, 316865, 316866, 316867, 316868) c6( ) c7(eR); #c1(ANKFY1) c2(XP_011522235) c3(rabankyrin-5 isoform X7) c4( ) c5(316869) c6( ) c7(eR); #c1(ANKH) c2(NP_473368) c3(progressive ankylosis protein homolog) c4(230451, 230452) c5(230453, 230454, 230455, 230456, 230457, 230458, 230459, 230460, 230461, 230462, 230463, 230464) c6( ) c7(dP, VC, bA, ai, mR, cc, rw, ha, Az, VI, U, v, VG, VD, lS, VK, VQ, VH, sg, VF); #c1(ANKHD1) c2(NP_060217) c3(ankyrin repeat and KH domain-containing protein 1 isoform 1) c4(194781, 194782, 194783) c5(194784, 194785, 194786, 194787, 194788, 194789, 194790, 194791, 194792, 194793, 194794, 194795, 194796, 194797, 194798, 194799, 194800, 194801, 194802, 194803, 194804, 194805, 194806, 194807, 194808, 194809, 194810, 194811, 194812, 194813, 194814, 194815, 194816, 194817, 194818) c6( ) c7(ck, T, fd, hF, ae, ad); #c1(ANKHD1) c2(NP_060448) c3(ankyrin repeat and KH domain-containing protein 1 isoform 2) c4(195986) c5(195987, 195988, 195989, 195990, 195991, 195992) c6( ) c7(ck, T, fd, hF, ae, ad); #c1(ANKHD1) c2(NP_078944) c3(ankyrin repeat and KH domain-containing protein 1 isoform 3) c4(216216) c5(216217, 216218, 216219, 216220, 216221, 216222) c6( ) c7(ck, T, fd, hF, ae, ad); #c1(ANKHD1-EIF4EBP3) c2(NP_065741) c3(ANKHD1-EIF4EBP3 protein) c4(205137, 205138, 205139, 205140) c5(205141, 205142, 205143, 205144, 205145, 205146, 205147, 205148, 205149, 205150, 205151, 205152, 205153) c6( ) c7(hF, T, ae, ad, fd); #c1(ANKK1) c2(NP_848605) c3(ankyrin repeat and protein kinase domain-containing protein 1) c4(253949) c5(253950, 253951, 253952, 253953, 253954, 253955) c6( ) c7(Gl, uk, VR, rG, jd, He, ht, VT, bO, Gd, hy, bS, uu, ct, v, W, aR, no, il, Gw, dL, bK, K, oi, he, na, hZ, Jj, kl, Gv, Y, Z, jb, tl, FT, hq, pH, VS, ag, Gs, VU, sv, Gu, lB); #c1(ANKLE1) c2(NP_001265373) c3(ankyrin repeat and LEM domain-containing protein 1 isoform 3) c4(98383) c5(98384, 98385, 98386, 98387, 98388, 98389, 98390, 98391, 98392, 98393, 98394, 98395, 98396, 98397, 98398) c6( ) c7(t, aT); #c1(ANKLE1) c2(NP_689576) c3(ankyrin repeat and LEM domain-containing protein 1 isoform 1) c4(243/11, 243112) c5(243113, 243114, 243115, 243116, 243117, 243118, 243119, 243120, 243121, 243122) c6( ) c7(t, aT); #c1(ANKLE2) c2(NP_055929) c3(ankyrin repeat and LEM domain-containing protein 2) c4(184497) c5(184498, 184499, 184500, 184501, 184502, 184503, 184504) c6( ) c7(y, TC); #c1(ANKLE2) c2(XP_005266216) c3(ankyrin repeat and LEM domain-containing protein 2 isoform X1) c4(279950) c5(279951, 279952, 279953, 279954, 279955) c6( ) c7(y, TC); #c1(ANKLE2) c2(XP_011533090) c3(ankyrin repeat and LEM domain-containing protein 2 isoform X3) c4(329281) c5(329282, 329283, 329284, 329285, 329286, 329287) c6( ) c7(y, TC); #c1(ANKMY1) c2(NP_001269700) c3(ankyrin repeat and MYND domain-containing protein 1 isoform 3) c4( ) c5(102602) c6( ) c7(af); #c1(ANKMY1) c2(NP_001269710) c3(ankyrin repeat and MYND domain-containing protein 1 isoform 5) c4(102676) c5(102677, 102678, 102679, 102680, 102681) c6( ) c7(af); #c1(ANKMY1) c2(NP_057636) c3(ankyrin repeat and MYND domain-containing protein 1 isoform 1) c4( ) c5(191994, 191995, 191996, 191997, 191998, 191999, 192000, 192001) c6( ) c7(af); #c1(ANKMY1) c2(NP_060314) c3(ankyrin repeat and MYND domain-containing protein 1 isoform 2) c4( ) c5(195372, 195373, 195374, 195375, 195376, 195377, 195378, 195379, 195380, 195381, 195382, 195383, 195384, 195385, 195386, 195387) c6( ) c7(af); #c1(ANKMY1) c2(XP_011509603) c3(ankyrin repeat and MYND domain-containing protein 1 isoform X2) c4(300177) c5(300178) c6( ) c7(af); #c1(ANKMY1) c2(XP_011509615) c3(ankyrin repeat and MYND domain-containing protein 1 isoform X12) c4(300179, 300180) c5( ) c6( ) c7(af); #c1(ANKMY1) c2(XP_011509620) c3(ankyrin repeat and MYND domain-containing protein 1 isoform X18) c4(300181) c5(300182, 300183) c6( ) c7(af); #c1(ANKMY1) c2(XP_011509621) c3 (ankyrin repeat and MYND domain-containing protein 1 isoform X19) c4( ) c5(300184, 300185, 300186, 300187, 300188, 300189, 300190, 300191, 300192, 300193, 300194, 300195, 300196) c6( ) c7(af); #c1(ANKMY1) c2(XP_011509624) c3 (ankyrin repeat and MYND domain-containing protein 1 isoform X22) c4(300197, 300198) c5(300199, 300200, 300201, 300202, 300203, 300204) c6( ) c7(af); #c1(ANKRD1) c2(NP_055206) c3(ankyrin repeat domain-containing protein 1) c4(179819) c5(179820, 179821, 179822, 179823, 179824, 179825) c6( ) c7(T, nA, cd, dP, bv, cc, Bc, dE, vc, K, aq, VW, g, t, c, wK, hc, mx, ll, m, im, bD, B); #c1(ANKRD10) c2(NP_001273650) c3(ankyrin repeat domain-containing protein 10 isoform 2) c4(105550) c5(105551, 105552, 105553, 105554, 105555) c6( ) c7(ey); #c1(ANKRD10) c2(NP_060134) c3(ankyrin repeat domain-containing protein 10 isoform 1) c4(194347) c5(194348, 194349, 194350, 194351, 194352, 194353, 194354) c6( ) c7(ey); #c1(ANKRD11) c2(NP_037407) c3(ankyrin repeat domain-containing protein 11) c4(175341, 175342) c5(175343, 175344, 175345, 175346, 175347, 175348, 175349, 175350, 175351, 175352) c6( ) c7(VY, VX, aW, aT, nA, rz, cd, bN); #c1(ANKRD11) c2(XP_006721247) c3(ankyrin repeat domain-containing protein 11 isoform X5) c4( ) c5(293896, 293897, 293898, 293899, 293900, 293901, 293902, 293903, 293904) c6( ) c7(VY, VX, aW, aT, nA, rz, cd, bN); #c1(ANKRD11) c2(XP_011521359) c3(ankyrin repeat domain-containing protein 11 isoform X4) c4(316188) c5(316189, 316190, 316191, 316192, 316193, 316194) c6( ) c7(VY, VX, aW, aT, nA, rz, cd, bN); #c1(ANKRD12) c2(NP_001190985) c3(ankyrin repeat domain-containing protein 12 isoform 2) c4(80400, 80401) c5(80402, 80403, 80404, 80405, 80406, 80407, 80408, 80409, 80410, 80411, 80412, 80413) c6( ) c7(aW, aC, aT); #c1(ANKRD12) c2(NP_056023) c3(ankyrin repeat domain-containing protein 12 isoform 1) c4(185207) c5(185208, 185209, 185210, 185211, 185212, 185213, 185214) c6( ) c7(aW, aC, aT); #c1(ANKRD12) c2(XP_005258153) c3(ankyrin repeat domain-containing protein 12 isoform X3) c4(275526) c5( ) c6( ) c7(aW, aC, aT); #c1(ANKRD12) c2(XP_011523940) c3(ankyrin repeat domain-containing protein 12 isoform X4) c4(318465) c5(318466, 318467, 318468, 318469, 318470, 318471) c6( ) c7(aW, aC, aT); #c1(ANKRD12) c2(XP_011523941) c3(ankyrin repeat domain-containing protein 12 isoform X5) c4(318472) c5(318473, 318474, 318475, 318476, 318477, 318478, 318479, 318480, 318481, 318482, 318483) c6( ) c7(aW, aC, aT); #c1(ANKRD18A) c2(NP_671728) c3(ankyrin repeat domain-containing protein 18A) c4(242167) c5(242168, 242169, 242170, 242171, 242172, 242173) c6( ) c7(av); #c1(ANKRD18A) c2(XP_011516135) c3(ankyrin repeat domain-containing protein 18A isoform X4) c4(309355) c5( ) c6( ) c7(av); #c1(ANKRD18A) c2(XP_011516142) c3(ankyrin repeat domain-containing protein 18A isoform X11) c4(309356) c5( ) c6( ) c7(av); #c1(ANKRD18A) c2(XP_011516143) c3(ankyrin repeat domain-containing protein 18A isoform X12) c4( ) c5(309357, 309358, 309359, 309360, 309361, 309362, 309363, 309364, 309365, 309366, 309367, 309368) c6( ) c7(av); #c1(ANKRD2) c2(NP_001123453) c3(ankyrin repeat domain-containing protein 2 isoform b) c4(47100, 47101) c5(47102, 47103, 47104, 47105, 47/06, 47107, 47108, 47109, 47110, 47111, 47112) c6( ) c7(bv, VZ, c, oe, aj, fr, J); #c1(ANKRD2) c2(NP_001278147) c3(ankyrin repeat domain-containing protein 2 isoform c) c4( ) c5(109484, 109485) c6( ) c7(bv, VZ, c, oe, aj, fr, J); #c1(ANKRD2) c2(NP_001278148) c3(ankyrin repeat domain-containing protein 2 isoform d) c4( ) c5(109486) c6( ) c7(bv, VZ, c, oe, aj, fr, J); #c1(ANKRD2) c2(NP_065082) c3(ankyrin repeat domain-containing protein 2 isoform a) c4(203764, 203765) c5(203766, 203767, 203768, 203769, 203770, 203771, 203772, 203773, 203774, 203775) c6( ) c7(bv, VZ, c, oe, aj, fr, J); #c1(ANKRD23) c2(NP_659431) c3(ankyrin repeat domain-containing protein 23) c4(239842, 239843) c5(239844, 239845, 239846, 239847, 239848, 239849, 239850, 239851, 239852, 239853, 239854, 239855, 239856, 239857, 239858, 239859) c6( ) c7(bL); #c1(ANKRD26) c2(NP_055730) c3(ankyrin repeat domain-containing protein 26 isoform 1) c4(183159, 183160) c5(183161, 183162, 183163, 183164, 183165, 183166) c6( ) c7(ck, hF, dX, v, Wa); #c1(ANKRD26) c2(XP_0067/7491) c3(ankyrin repeat domain-containing protein 26 isoform X10) c4(291147) c5(291148, 291149, 291150, 291151, 291152, 291153) c6( ) c7(ck, hF, dX, v, Wa); #c1(ANKRD26) c2(XP_011517725) c3(ankyrin repeat domain-containing protein 26 isoform X9) c4(311508) c5( ) c6( ) c7(ck, hF, dX, v, Wa); #c1(ANKRD26) c2(XP_011517726) c3(ankyrin repeat domain-containing protein 26 isoform X12) c4( ) c5(311509, 311510, 311511, 311512, 311513, 311514, 311515, 311516, 311517, 311518, 311519, 311520) c6( ) c7(ck, hF, dX, v, Wa); #c1(ANKRD28) c2(NP_056014) c3(serine/threonine-protein phosphatase 6 regulatory ankyrin repeat subunit A isoform a) c4(185140) c5(185141, 185142, 185143, 185144, 185145, 185146, 185147, 185148, 185149, 185150) c6( ) c7(bt); #c1(ANKRD28) c2(XP_011531842) c3(serine/threonine-protein phosphatase 6 regulatory ankyrin repeat subunit A isoform X2) c4(327911) c5(327912, 327913, 327914, 327915, 327916, 327917) c6( ) c7(bt); #c1(ANKRD30A) c2(NP_443723) c3(ankyrin repeat domain-containing protein 30A) c4(230017) c5(230018, 230019, 230020, 230021, 230022, 230023) c6( ) c7(T, aW, aT, ci, AO, ad); #c1(ANKRD36) c2(NP_001157787) c3(ankyrin repeat domain-containing protein 36A) c4(62652, 62653, 62654) c5(62655, 62656, 62657, 62658, 62659, 62660, 62661, 62662, 62663, 62664, 62665, 62666, 62667, 62668, 62669, 62670, 62671) c6( ) c7(fq, VM); #c1(ANKRD36) c2(XP_006712577) c3(ankyrin repeat domain-containing protein 36A isoform X1) c4(286729) c5(286730) c6( ) c7(fq, VM); #c1(ANKRD36) c2(XP_006712579) c3(ankyrin repeat domain-containing protein 36A isoform X2) c4(286731) c5( ) c6( ) c7(fq, VM); #c1(ANKRD36) c2(XP_011509437) c3(ankyrin repeat domain-containing protein 36A isoform X6) c4(300020) c5( ) c6( ) c7(fq, VM); #c1(ANKRD36) c2(XP_011509438) c3(ankyrin repeat domain-containing protein 36A isoform X7) c4(300021) c5( ) c6( ) c7(fq, VM); #c1(ANKRD36) c2(XP_011509439) c3(ankyrin repeat domain-containing protein 36A isoform X8) c4(300022) c5( ) c6( ) c7(fq, VM); #c1(ANKRD36) c2(XP_011509447) c3(ankyrin repeat domain-containing protein 36A isoform X13) c4( ) c5(300023, 300024, 300025, 300026, 300027, 300028, 300029, 300030, 300031, 300032, 300033, 300034, 300035, 300036, 300037, 300038) c6( ) c7(fq, VM); #c1(ANKRD36B) c2(NP_079466) c3(ankyrin repeat domain-containing protein 36B) c4( ) c5(218208) c6( ) c7(T, dA, v, ci, eA, q, hd, aT, d, aW, Y, oV, ff, r, aq, en, bu, bz, iP, eM, nB, eD, B); #c1(ANKRD36B) c2(XP_011509847) c3(ankyrin repeat domain-containing protein 368 isoform X4) c4(300436) c5( ) c6( ) c7(T, dA, v, ci, eA, q, hd, aT, d, aW, Y, oV, ff, r, aq, en, bu, bz, iP, eM, nB, eD, B); #c1(ANKRD36B) c2(XP_011509854) c3 (ankyrin repeat domain-containing protein 36B isoform X11) c4( ) c5(300437, 300438, 300439, 300440, 300441, 300442, 300443, 300444, 300445, 300446, 300447) c6( ) c7(T, dA, v, ci, eA, q, hd, aT, d, aW, Y, oV, ff, r, aq, en, bu, bz, iP, eM, nB, eD, B); #c1(ANKRD37) c2(NP_859077) c3(ankyrin repeat domain-containing protein 37) c4(255515, 255516) c5(255517, 255518, 255519, 255520, 255521, 255522, 255523, 255524, 255525, 255526, 255527, 255528) c6( ) c7(rx); #c1(ANKRD44) c2(NP_001182073) c3(serine/threonine-protein phosphatase 6 regulatory ankyrin repeat subunit B isoform A) c4(74559, 74560) c5(74561, 74562, 74563, 74564, 74565, 74566, 74567, 74568, 74569, 74570, 74571, 74572, 74573, 74574, 74575, 74576, 74577, 74578, 74579, 74580, 74581, 74582, 74583, 74584, 74585, 74586, 74587, 74588, 74589, 74590, 74591, 74592, 74593, 74594) c6( ) c7(Wb, Ea); #c1(ANKRD44) c2(NP_710181) c3(serine/threonine-protein phosphatase 6 regulatory ankyrin repeat subunit B isoform B) c4( ) c5(246548, 246549, 246550, 246551, 246552, 246553) c6( ) c7(Wb, Ea); #c1(ANKRD44) c2(XP_005247002) c3(serine/threonine-protein phosphatase 6 regulatory ankyrin repeat subunit B isoform X1) c4(267589) c5( ) c6( ) c7(Wb, Ea); #c1(ANKRD44) c2(XP_005247003) c3(serine/threonine-protein phosphatase 6 regulatory ankyrin repeat subunit B isoform X2) c4(267590, 267591, 267592) c5( ) c6( ) c7(Wb, Ea); #c1(ANKRD45) c2(XP_011507772) c3(ankyrin repeat domain-containing protein 45 isoform X1) c4(297836) c5(297837, 297838, 297839, 297840, 297841, 297842) c6( ) c7(W); #c1(ANKRD46) c2(NP_001257308) c3(ankyrin repeat domain-containing protein 46 isoform 2) c4(95114, 95115, 95116, 95117, 95118) c5(95119, 95120, 95121, 95122, 95123, 95124) c6( ) c7(T); #c1(ANKRD46) c2(XP_006716572) c3(ankyrin repeat domain-containing protein 46 isoform X1) c4(290380) c5(290381, 290382, 290383, 290384, 290385, 290386, 290387, 290388, 290389, 290390, 290391, 290392, 290393, 290394, 290395, 290396, 290397, 290398, 290399, 290400, 290401, 290402, 290403, 290404, 290405, 290406, 290407, 290408, 290409, 290410, 290411, 290412, 290413, 290414, 290415, 290416, 290417, 290418) c6( ) c7(T); #c1(ANKRD50) c2(NP_001161354) c3(ankyrin repeat domain-containing protein 50 isoform 2) c4(65631) c5(65632, 65633, 65634, 65635, 65636, 65637) c6( ) c7(fq); #c1(ANKRD50) c2(NP_065070) c3(ankyrin repeat domain-containing protein 50 isoform 1) c4(203609) c5(203610, 203611, 203612, 203613, 203614, 203615, 203616, 203617) c6( ) c7(fq); #c1(ANKRD55) c2(NP_078945) c3(ankyrin repeat domain-containing protein 55) c4(216223, 216224) c5(216225, 216226, 216227, 216228, 216229, 216230, 216231, 216232, 216233, 216234) c6( ) c7(bL, cz, NI, y, V, ad); #c1(ANKRD55) c2(XP_006714754) c3(ankyrin repeat domain-containing protein 55 isoform X1) c4( ) c5(288099, 288100, 288101, 288102, 288103, 288104) c6( ) c7(bL, cz, NI, y, V, ad); #c1(ANKRD6) c2(NP_001229738) c3(ankyrin repeat domain-containing protein 6 isoform a) c4(85008, 85009, 85010) c5(85011, 85012, 85013, 85014, 85015, 85016, 85017, 85018, 85019, 85020) c6( ) c7(aw, ac, eM); #c1(ANKRD6) c2(NP_001229742) c3(ankyrin repeat domain-containing protein 6 isoform c) c4(85021) c5(85022, 85023, 85024, 85025, 85026, 85027, 85028, 85029, 85030, 85031, 85032, 85033, 85034, 85035, 85036, 85037, 85038, 85039) c6( ) c7(aw, ac, eM); #c1(ANKRD6) c2(NP_001229743) c3(ankyrin repeat domain-containing protein 6 isoform d) c4(85040, 85041) c5(85042, 85043, 85044, 85045, 85046, 85047, 85048, 85049, 85050, 85051, 85052, 85053, 85054, 85055, 85056, 85057, 85058, 85059) c6( ) c7(aw, ac, eM); #c1(ANKRD6) c2(NP_055757) c3(ankyrin repeat domain-containing protein 6 isoform b) c4(183322) c5(183323, 183324, 183325, 183326, 183327, 183328) c6( ) c7(aw, ac, eM); #c1(ANKRD6) c2(XP_011533901) c3(ankyrin repeat domain-containing protein 6 isoform X9) c4(330103) c5( ) c6( ) c7(aw, ac, eM); #c1(ANKRD7) c2(NP_062618) c3(ankyrin repeat domain-containing protein 7) c4(202035, 202036, 202037) c5(202038, 202039, 202040, 202041, 202042, 202043, 202044, 202045, 202046, 202047, 202048, 202049, 202050, 202051, 202052, 202053, 202054, 202055) c6( ) c7(aC, fq, j, Ea, sj); #c1(ANKS1A) c2(NP_056060) c3(ankyrin repeat and SAM domain-containing protein (A) c4(185439, 185440, 185441, 185442) c5(185443, 185444, 185445, 185446, 185447, 185448, 185449, 185450, 185451, 185452, 185453, 185454, 185455, 185456, 185457, 185458, 185459, 185460, 185461, 185462, 185463, 185464, 185465) c6( ) c7(j, eZ); #c1(ANKS1A) c2(XP_011512739) c3(ankyrin repeat and SAM domain-containing protein IA isoform X9) c4( ) c5(303768) c6( ) c7(j, eZ); #c1(ANKS1B) c2(NP_001190995) c3(ankyrin repeat and sterile alpha motif domain-containing protein 18 isoform e) c4(80455, 80456) c5(80457, 80458, 80459, 80460, 80461, 80462, 80463, 80464, 80465, 80466, 80467) c6( ) c7(ck, bF, ou, FO, e, aw, FP, EQ, r, aj, k, jj, fq); #c1(ANKS1B) c2(NP_001190996) c3(ankyrin repeat and sterile alpha motif domain-containing protein (B isoform f) c4( ) c5(80468, 80469, 80470, 80471, 80472, 80473) c6( ) c7(ck, bF, ou, FO, e, aw, FP, EQ, r, aj, k, jj, fq); #c1(ANKS1B) c2(NP_001190997) c3(ankyrin repeat and sterile alpha motif domain-containing protein 1B isoform g) c4(80474) c5(80475, 80476, 80477, 80478, 80479, 80480) c6( ) c7(ck, bF, ou, FO, e, aw, FP, EQ, r, aj, k, jj, fq); #c1(ANKS1B) c2(NP_001190998) c3(ankyrin repeat and sterile alpha motif domain-containing protein (B isoform h) c4(80481, 80482) c5(80483, 80484, 80485, 80486, 80487, 80488, 80489, 80490, 80491, 80492, 80493, 80494, 80495) c6( ) c7(ck, bF, ou, FO, e, aw, FP, EQ, r, aj, k, jj, fq); #c1(ANKS1B) c2(NP_001190999) c3(ankyrin repeat and sterile alpha motif domain-containing protein 18 isoform i) c4(80496) c5(80497, 80498, 80499, 80500, 80501, 80502) c6( ) c7(ck, bF, ou, FO, e, aw, FP, EQ, r, aj, k, jj, fq); #c1(ANKS1B) c2(NP_001191010) c3(ankyrin repeat and sterile alpha motif domain-containing protein 1B isoform I) c4( ) c5(80544, 80545, 80546, 80547, 80548) c6( ) c7(ck, bF, ou, FO, e, aw, FP, EQ, r, aj, k, jj, fq); #c1(ANKS1B) c2(NP_064525) c3(ankyrin repeat and sterile alpha motif domain-containing protein (B isoform c) c4(202864) c5(202865, 202866, 202867, 202868, 202869, 202870) c6( ) c7(ck, bF, ou, FO, e, aw, FP, EQ, r, aj, k, jj, fq); #c1(ANKS1B) c2(NP_690001) c3(ankyrin repeat and sterile alpha motif domain-containing protein 18 isoform a) c4(244688, 244689, 244690) c5(244691, 244692, 244693, 244694, 244695, 244696, 244697, 244698, 244699, 244700, 244701, 244702, 244703) c6( ) c7(ck, bF, ou, FO, e, aw, FP, EQ, r, aj, k, jj, fq); #c1(ANKS1B) c2(NP_858056) c3(ankyrin repeat and sterile alpha motif domain-containing protein IB isoform b) c4(255319) c5(255320, 255321, 255322, 255323, 255324, 255325) c6( ) c7(ck, bF, ou, FO, e, aw, FP, EQ, r, aj, k, jj, fq); #c1(ANKS1B) c2(XP_006719576) c3(ankyrin repeat and sterile alpha motif domain-containing protein (B isoform X16) c4(292639) c5( ) c6( ) c7(ck, bF, ou, FO, e, aw, FP, EQ, r, aj, k, jj, fq); #c1(ANKS1B) c2(XP_006719577) c3(ankyrin repeat and sterile alpha motif domain-containing protein 18 isoform X17) c4(292640, 292641) c5(292642, 292643, 292644, 292645, 292646, 292647, 292648, 292649, 292650, 292651, 292652, 292653) c6( ) c7(ck, bF, ou, FO, e, aw, FP, EQ, r, aj, k, jj, fq); #c1(ANKS4B) c2(NP_665872) c3(ankyrin repeat and SAM domain-containing protein 4B) c4(241878) c5(241879, 241880, 241881, 241882, 241883, 241884) c6( ) c7(Pw, Pv, Pu); #c1(ANKS6) c2(NP_775822) c3(ankyrin repeat and SAM domain-containing protein 6) c4(250425, 250426) c5(250427, 250428, 250429, 250430, 250431, 250432, 250433, 250434, 250435, 250436, 250437, 250438) c6( ) c7(Na, eR, BV, al, vm, Nc, aY, Wc); #c1(ANKS6) c2(XP_006717061) c3(ankyrin repeat and SAM domain-containing protein 6 isoform X3) c4(290671) c5(290672, 290673, 290674, 290675, 290676, 290677) c6( ) c7(Na, eR, BV, al, vm, Nc, aY, Wc); #c1(ANLN) c2(NP_001271230) c3(actin-binding protein anillin isoform 2) c4(103666) c5(103667, 103668, 103669, 103670, 103671, 103672) c6( ) c7(eM, kl, fq, B, bz); #c1(ANLN) c2(NP_061155) c3(actin-binding protein anillin isoform 1) c4(199631) c5(199632, 199633, 199634, 199635, 199636, 199637) c6( ) c7(eM, kl, fq, B, bz); #c1(ANO1) c2(NP_060513) c3(anoctamin-1) c4(196302, 196303) c5(196304, 196305, 196306, 196307, 196308, 196309, 196310, 196311, 196312, 196313, 196314, 196315) c6( ) c7(bM, T, pa, QG, hW, op, aW, iW, Wf, Ls, vA, aT, aA, Wd, ao, aF, ci, fb, ik, iJ, We); #c1(ANDI) c2(XP_006718668) c3(anoctamin-1 isoform X12) c4(291951) c5(291952, 291953, 291954, 291955, 291956, 291957, 291958, 291959, 291960, 291961, 291962, 291963, 291964, 291965) c6( ) c7(bM, T, pa, QG, hW, op, aW, iW, Wf, Ls, vA, aT, aA, Wd, ao, aF, ci, fb, ik, iJ, We): #c1(ANO1) c2(XP_011543429) c3(anoctamin-1 isoform X7) c4(342320) c5(342321, 342322, 342323, 342324, 342325, 342326) c6( ) c7(bM, T, pa, QG, hW, op, aW, iW, Wf, Ls, vA, aT, aA, Wd, ao, aF, ci, fb, ik, iJ, We); #c1(ANO1) c2(XP_011543430) c3(anoctamin-1 isoform X8) c4( ) c5(342327, 342328) c6( ) c7(bM, T, pa, QG, hW, op, aW, iW, Wf, Ls, vA, aT, aA, Wd, ao, af, ci, fb, iK, iJ, We); #c1(ANO1) c2(XP_011543432) c3 (anoctamin-1 isoform X11) c4(342329) c5( ) c6( ) c7(bM, T, pa, QG, hW, op, aW, iW, Wf, Ls, vA, aT, aA, Wd, ao, aF, ci, fb, ik, iJ, We); #c1(ANO1) c2(XP_011543433) c3(anoctamin-1 isoform X13) c4(342330) c5(342331, 342332, 342333, 342334, 342335, 342336) c6( ) c7(bM, T, pa, QG, hW, op, aW, iW, Wf, Ls, vA, aT, aA, Wd, ao, aF, ci, fb, ik, iJ, We); #c1(ANO10) c2(NP_001191760) c3(anoctamin-10 isoform 2) c4(81670) c5(81671, 81672, 81673, 81674, 81675, 81676) c6( ) c7(Wg, ac, ky); #c1(ANO10) c2(NP_001191761) c3(anoctamin-10 isoform 3) c4(81677) c5(81678, 81679, 81680, 81681, 81682, 81683) c6( ) c7(Wg, ac, ky); #c1(ANO10) c2(NP_001191762) c3(anoctamin-10 isoform 4) c4(81684) c5(81685, 81686, 81687, 81688, 81689, 81690) c6( ) c7(Wg, ac, ky); #c1(ANO10) c2(NP_00119 (763) c3(anoctamin-10 isoform 5) c4(81691, 81692, 81693, 81694, 81695, 81696) c5(81697, 81698, 81699, 81700, 81701, 81702, 81703, 81704, 81705, 81706, 81707, 81708, 81709, 81710, 81711, 81712, 81713, 81714, 81715, 81716, 81717, 81718, 81719, 81720, 81721, 81722, 81723, 81724, 81725, 81726, 81727, 81728, 81729, 81730, 81731, 81732, 81733, 81734, 81735, 81736, 81737, 81738) c6( ) c7(Wg, ac, ky); #c1(ANO10) c2(NP_060545) c3(anoctamin-10 isoform 1) c4(196492) c5(196493, 196494, 196495, 196496, 196497, 196498) c6( ) c7(Wg, ac, ky); #c1(ANO10) c2(XP_011532185) c3(anoctamin-10 isoform X1) c4(328232) c5( ) c6( ) c7(Wg, ac, ky); #c1(ANO2) c2(NP_001265525) c3(anoctamin-2 isoform 1) c4(98733) c5(98734, 98735, 98736, 98737, 98738, 98739) c6( ) c7(qk, Wh, ww); #c1(ANO2) c2(NP_001265526) c3(anoctamin-2 isoform 2) c4(98740, 98741, 98742) c5(98743, 98744, 98745, 98746, 98747, 98748, 98749, 98750, 98751, 98752, 98753, 98754, 98755, 98756, 98757, 98758, 98759, 98760) c6( ) c7(qk, Wh, ww); #c1(ANO2) c2(XP_011519280) c3(anoctamin-2 isoform X5) c4(313920) c5( ) c6( ) c7(qk, Wh, ww); #c1(ANO2) c2(XP_011519287) c3(anoctamin-2 isoform X11) c4( ) c5(313921, 313922, 313923, 313924, 313925, 313926) c6( ) c7(qk, Wh, ww); #c1(ANO3) c2(NP_113606) c3(anoctamin-3) c4(221689, 221690, 221691) c5(221692, 221693, 221694, 221695, 221696, 221697) c6( ) c7(Gt, Wi, Wk, aU, v, j, WI, Wm, Wj, fq, aH); #c1(ANO4) c2(NP_001273545) c3(anoctamin-4 isoform 1) c4( ) c5(105153, 105154, 105155, 105156, 105157, 105158, 105159, 105160, 105161, 105162, 105163, 105164, 105165, 105166) c6( ) c7(dP, lB, fq, cd); #c1(ANO4) c2(NP_849148) c3(anoctamin-4 isoform 2) c4(254294) c5(254295, 254296, 254297, 254298, 254299) c6( ) c7(dP, lB, fq, cd); #c1(ANO4) c2(XP_011536214) c3(anoctamin-4 isoform X2) c4(333527) c5(333528, 333529, 333530, 333531) c6( ) c7(dP, lB, fq, cd); #c1(ANO4) c2(XP_011536217) c3(anoctamin-4 isoform X4) c4(333532, 333533) c5( ) c6( ) c7(dP, lB, fq, cd); #c1(ANO4) c2(XP_011536218) c3 (anoctamin-4 isoform X5) c4( ) c5(333534, 333535, 333536, 333537) c6( ) c7(dP, lB, fq, cd); #c1(ANO4) c2(XP_011536220) c3(anoctamin-4 isoform X8) c4( ) c5(333538, 333539, 333540, 333541, 333542) c6( ) c7(dP, lB, fq, cd); #c1(ANO5) c2(NP_998764) c3(anoctamin-5 isoform a) c4(265875) c5(265876, 265877, 265878, 265879, 265880, 265881) c6( ) c7(cc, Rk, Wo, Wn, Ws, FB, xs, mx, oj, Ak, km, Wd, Wp, R, Wr, H, Wq, lf); #c1(ANO6) c2(NP_001020527) c3(anoctamin-6 isoform a) c4(28828) c5(28829, 28830, 28831, 28832, 28833, 28834) c6( ) c7(dY, mR, jC); #c1(ANO6) c2(NP_001136150) c3(anoctamin-6 isoform b) c4(54425) c5(54426, 54427, 54428, 54429, 54430, 54431) c6( ) c7(dY, mR, jC); #c1(ANO6) c2(NP_001136151) c3(anoctamin-6 isoform c) c4(54432) c5(54433, 54434, 54435, 54436, 54437, 54438, 54439, 54440, 54441, 54442) c6( ) c7(dY, mR, jC); #c1(ANO6) c2(NP_001191732) c3(anoctamin-6 isoform d) c4(81627) c5(81628, 81629, 81630, 81631, 81632, 81633, 81634) c6( ) c7(dY, mR, jC); #c1(ANO7) c2(NP_001001666) c3(anoctamin-7 isoform NGEP-short) c4(18943) c5(18944, 18945, 18946, 18947, 18948, 18949) c6( ) c7(T, ae, ad, g); #c1(ANO7) c2(NP_001001891) c3 (anoctamin-7 isoform NGEP-long) c4(19105) c5(19106, 19107, 19108, 19109, 19110, 19111, 19112) c6( ) c7(T, ae, ad, g); #c1(ANO7) c2(XP_011509558) c3(anoctamin-7 isoform X1) c4( ) c5(300136) c6( ) c7(T, ae, ad, g); #c1(ANO7) c2(XP_011509565) c3(anoctamin-7 isoform X8) c4(300137) c5(300138, 300139, 300140, 300141, 300142, 300143) c6( ) c7(T, ae, ad, g); #c1(ANO7) c2(XP_011509572) c3(anoctamin-7 isoform X15) c4( ) c5(300144) c6( ) c7(T, ae, ad, g); #c1(AN07) c2(XP_011509575) c3(anoctamin-7 isoform X18) c4( ) c5(300145, 300146, 300147, 300148, 300149) c6( ) c7(T, ae, ad, g); #c1(ANP32A) c2(NP_006296) c3(acidic leucine-rich nuclear phosphoprotein 32 family member A) c4(163773, 163774) c5(163775, 163776, 163777, 163778, 163779, 163780, 163781, 163782, 163783, 163784, 163785, 163786, 163787) c6( ) c7(ae, T, av, aE, aW, aT, aq, Kh, ci, AD, KM, B, ad); #c1(ANP32B) c2(NP_006392) c3(acidic leucine-rich nuclear phosphoprotein 32 family member B) c4(164649, 164650) c5(164651, 164652, 164653, 164654, 164655, 164656, 164657, 164658, 164659, 164660, 164661, 164662) c6( ) c7(bM, T, fM, aw, ex, fd, j, V, eA, q, Wu, h, eV, ji, d, s, cW, ff, eZ, r, aF, en, eE, ve, Wt, bE, cj, eo, lf, y); #c1(ANP32D) c2(NP_036536) c3(acidic leucine-rich nuclear phosphoprotein 32 family member D) c4(174271) c5(174272, 174273, 174274, 174275, 174276, 174277) c6( ) c7(ae, ci, ad); #c1(ANPEP) c2(NP_001141) c3(aminopeptidase N precursor) c4(60068, 60069, 60070) c5(60071, 60072, 60073, 60074, 60075, 60076) c6( ) c7(Wx, T, dD, dA, fR, Wz, ea, rd, WA, DA, bs, cz, bB, bO, v, eq, ci, Ww, pl, fd, aY, hF, cd, br, ad, ip, x, av, eW, hd, GL, Wy, WB, aT, aU, cy, iY, ji, ox, jj, ae, bc, iL, cY, iU, xR, aW, Y, xs, hl, eH, aq, lo, jh, hp, g, Fu, hJ, eF, iR, bw, Wv, ck, eM, nB, bE, cj, m, k, y, bt, B, ps); #c1(ANPEP) c2(XP_011519776) c3(aminopeptidase N isoform X2) c4( ) c5(314455, 314456, 314457, 314458, 314459, 314460, 314461, 314462, 314463, 314464, 314465, 314466) c6( ) c7(Wx, T, dD, dA, FR, Wz, ea, rQ, WA, oA, bs, cz, bB, bO, v, eq, ci, Ww, pl, fd, aY, hF, cd, br, ad, ip, x, av, eW, hd, GL, Wy, WB, aT, aU, cy, iY, ji, ox, jj, ae, bc, iL, cY, iU, xR, aW, Y, xs, hl, eH, aq, lo, jh, hp, g, Fu, hJ, eF, iR, bw, Wv, ck, eM, nB, bE, cj, m, k, y, bt, B, ps); #c1(ANTXR1) c2(NP_060623) c3(anthrax toxin receptor 1 isoform 3 precursor) c4(196877) c5(196878, 196879, 196880, 196881, 196882, 196883) c6( ) c7(DS, T, os, dD, aC, fR, id, ea, aw, ew, aA, bB, WD, oZ, jh, j, eq, nA, WC, q, ad, hb, WG, aT, h, By, K, WF, cY, sb, d, kj, wr, aW, WE, Y, ff, r, nf, kp, aF, WJ, en, t, bz, WH, W, qe, bE, WK, cj, Km, m, ER, np, WI, bC, WL); #c1(ANTXR1) c2(NP_115584) c3(anthrax toxin receptor 1 isoform 1 precursor) c4(223281) c5(223282, 223283, 223284, 223285, 223286, 223287) c6( ) c7(DS, T, os, dD, aC, fR, id, ea, aw, ew, aA, bB, WD, oZ, jh, j, eq, nA, WC, q, ad, hb, WG, aT, h, ey, K, WF, cY, sb, d, kj, wr, aW, WE, Y, ff, r, nf, kp, aF, WJ, en, t, bz, WH, W, qe, bE, WK, cj, Km, m, ER, np, WI, bC, WL); #c1(ANTXR1) c2(NP_444262) c3 (anthrax toxin receptor 1 isoform 2 precursor) c4(230168) c5(230169, 230170, 230171, 230172, 230173, 230174) c6( ) c7(DS, T, os, dD, aC, fR, id, ea, aw, ew, aA, bB, WD, oZ, jh, j, eq, nA, WC, q, ad, hb, WG, aT, h, ey, K, WF, cY, sb, d, kj, wr, aW, WE, Y, ff, r, nf, kp, aF, WJ, en, t, bz, WH, W, qe, bE, WK, cj, Km, m, ER, np, WI, bC, WL); #c1(ANTXR2) c2(NP_001139266) c3(anthrax toxin receptor 2 isoform 2 precursor) c4(59036, 59037) c5(59038, 59039, 59040, 59041, 59042, 59043) c6( ) c7(DS, T, if, jh, aA, cz, qN, hX, lg, aT, pk, qk, bA, cY, mR, je, aW, lo, aF, WM, iM, wR, WN, bt); #c1(ANTXR2) c2(NP_477520) c3(anthrax toxin receptor 2 isoform 1 precursor) c4(230800, 230801) c5(230802, 230803, 230804, 230805, 230806, 230807, 230808, 230809, 230810, 230811, 230812, 230813, 230814, 230815, 230816, 230817, 230818, 230819) c6( ) c7(DS, T, if, jh, aA, cz, qN, hX, lg, aT, pk, qk, bA, cY, mR, je, aW, lo, aF, WM, iM, wR, WN, bt); #c1(ANXA1) c2(XP_011516910) c3(annexin A1 isoform X2) c4(310403, 310404) c5(310405, 310406, 310407, 310408, 310409, 310410, 310411, 310412, 310413) c6( ) c7(T, aC, pf, W, ci, aR, Bc, dV, hu, eL, aa, zQ, aW, Nz, Az, hl, Ku, g, as, eF, cm, WO, nB, WS, U, sw, ht, NT, fr, zP, qW, cd, BI, ft, ju, h, dL, A, fQ, fe, fq, kT, E, it, B, ek, bL, fM, Dh, fG, oe, WT, fZ, aA, cz, eA, zn, hb, aT, Oi, cy, bq, ae, af, eu, jf, Y, ev, fO, WQ, Pg, en, ck, ac, WP, hr, WU, dy, gT, rA, bt, ao, Gl, hl, id, jh, vr, Fb, Gn, Fc, ad, hX, dP, av, iC, hd, Db, yt, WR, ji, d, ff, xs, eH, eZ, Ej, Sm, iV, bz, ek, LB, D, y, xg, iu); #c1(ANXA1) c2(XP_011516911) c3(annexin A1 isoform X1) c4(310414) c5(310415, 310416, 310417, 310418, 310419, 310420, 310421, 310422, 310423, 310424, 310425, 310426, 310427) c6( ) c7(T, aC, pf, WV, ci, aR, Bc, dV, hu, eL, aa, zQ, aW, Nz, Az, hl, Ku, g, as, eF, cm, WO, nB, WS, U, sw, ht, NT, fr, zP, qW, cd, Bl, ft, ju, h, dL, A, fQ, fe, fq, KT, E, it, B, ek, bL, fM, Dh, fG, oe, WT, fZ, aA, cz, eA, zn, hb, aT, Oi, cy, bq, ae, af, eu, jf, Y, ev, fO, WQ, Pg, en, ck, ac, WP, hr, WU, dy, gT, rA, bt, ao, Gl, hl, id, jh, vr, Fb, Gn, Fc, ad, hX, dP, av, iC, hd, Db, yt, WR, ji, d, ff, xs, eH, eZ, Ej, Sm, iV, bz, ek, LB, D, y, xg, iu); #c1(ANXA10) c2(NP_009124) c3(annexin A10) c4(170882, 170883) c5(170884, 170885, 170886, 170887, 170888, 170889, 170890, 170891, 170892, 170893, 170894, 170895) c6( ) c7(B, BO, s, bE, pf, aA, bB, n, aF, ci, g, q, WW); #c1(ANXA10) c2(XP_011529873) c3(annexin A10 isoform X1) c4( ) c5(325647) c6( ) c7(B, BO, s, bE, pf, aA, bB, n, aF, ci, g, q, WW); #c1(ANXA11) c2(NP_001265336) c3(annexin A11 isoform 1) c4(98282) c5( ) c6( ) c7(A, cL, hp, m, t, gb, o, uQ); #c1(ANXA11) c2(NP_001265338) c3(annexin A11 isoform 2) c4(98283, 98284) c5(98285, 98286, 98287, 98288, 98289, 98290, 98291, 98292, 98293, 98294, 98295, 98296, 98297, 98298, 98299, 98300, 98301, 98302, 98303, 98304, 98305, 98306, 98307, 98308, 98309, 98310, 98311, 98312, 98313, 98314, 98315, 98316, 98317, 98318, 98319, 98320, 98321, 98322, 98323, 98324, 98325, 98326, 98327, 98328, 98329, 98330, 98331, 98332, 98333, 98334, 98335, 98336, 98337, 98338, 98339, 98340, 98341, 98342, 98343, 98344, 98345) c6( ) c7(A, cL, hp, m, t, gb, D, uQ); #c1(ANXA11) c2(XP_005269798) c3(annexin A11 isoform X2) c4(282866, 282867) c5(282868) c6( ) c7(A, cL, hp, m, t, gb, o, uQ); #c1(ANXA11) c2(XP_005269799) c3(annexin A11 isoform X3) c4(282869, 282870) c5(282871, 282872, 282873, 282874, 282875, 282876) c6( ) c7(A, cl, hp, m, t, gb, o, uQ); #c1(ANXA13) c2(NP_001003954) c3(annexin A13 isoform b) c4(20352) c5(20353, 20354, 20355, 20356, 20357, 20358) c6( ) c7(T, aC, ac, dV, kk, ad); #c1(ANXA13) c2(NP_004297) c3(annexin A13 isoform a) c4(144959) c5(144960, 144961, 144962, 144963, 144964, 144965) c6( ) c7(T, aC, ac, dV, kk, ad); #c1(ANXA2) c2(NP_001002858) c3(annexin A2 isoform 1) c4(19651, 19652, 19653, 19654, 19655) c5(19656, 19657, 19658, 19659, 19660, 19661, 19662, 19663, 19664, 19665, 19666, 19667, 19668, 19669, 19670, 19671, 19672, 19673, 19674, 19675, 19676, 19677, 19678) c6( ) c7(bM, T, aC, ea, fd, bB, ci, x, AW, aE, dV, ja, K, hu, dF, zQ, aW, Du, hl, g, as, eF, cm, bw, eM, iA, iT, vy, k, m, dD, zt, ht, qy, ez, n, aj, fr, uE, cd, ft, cU, db, Um, pi, hi, ge, aq, kp, mo, dB, Vy, bm, qq, Xa, fL, B, Dw, dA, aA, j, eA, J, eJ, Mp, WY, aT, jj, bq, ae, bA, iZ, Y, en, cl, t, ik, ck, kP, WP, bt, vi, id, qb, eq, mC, ad, hX, dP, av, py, yt, pq, WZ, ul, gj, d, lR, cH, ff, xs, eH, cr, eZ, aF, bz, bE, ag, dX, aS, y, WX); #c1(ANXA2) c2(XP_011519778) c3(annexin A2 isoform X1) c4(314467, 314468, 314469, 314470, 314471, 314472, 314473, 314474) c5(314475, 314476, 314477, 314478, 314479, 314480, 314481, 314482, 314483, 314484, 314485, 314486, 314487, 314488, 314489, 314490, 314491, 314492, 314493, 314494, 314495, 314496, 314497, 314498, 314499, 314500, 314501, 314502, 314503, 314504, 314505, 314506, 314507, 314508, 314509, 314510, 314511, 314512, 314513, 314514, 314515, 314516, 314517, 314518, 314519, 314520, 314521, 314522, 314523, 314524, 314525, 314526, 314527, 314528, 314529, 314530, 314531, 314532, 314533, 314534, 314535, 314536, 314537, 314538, 314539, 314540, 314541, 314542, 314543, 314544, 314545, 314546, 314547, 314548, 314549) c6( ) c7(bM, T, aC, ea, fd, bB, ci, x, AW, aE, dV, ja, K, hu, dF, zQ, aW, Du, hl, g, as, eF, cm, bw, eM, iA, iT, vy, k, m, dD, zt, ht, qy, ez, n, aj, fr, uE, cd, ft, cU, db, Um, pi, hi, ge, aq, kp, mo, dB, Vy, bm, qq, Xa, fL, B, Dw, dA, aA, j, eA, J, eJ, Mp, WY, aT, jj, bq, ae, bA, iZ, Y, en, cl, t, ik, ck, kP, WP, bt, vi, id, qb, eq, mC, ad, hX, dP, av, py, yt, pq, WZ, ul, gj, d, lR, cH, ff, xs, eH, cr, eZ, aF, bz, bE, ag, dX, aS, y, WX); #c1(ANXA2R) c2(NP_001014301) c3(annexin-2 receptor) c4(25797) c5(25798, 25799, 25800, 25801, 25802, 25803) c6( ) c7(ad, fd); #c1(ANXA3) c2(NP_005130) c3(annexin A3) c4(153099, 153100, 153101, 153102) c5(153103, 153104, 153105, 153106, 153107, 153108, 153109, 153110, 153111, 153112, 153113, 153114, 153115, 153116, 153117, 153118, 153119, 153120, 153121, 153122, 153123, 153124, 153125, 153126) c6( ) c7(T, y, s, aW, eZ, aT, aA, HD, aF, m, dw, t, ad); #c1(ANXA4) c2(XP_011531107) c3(annexin A4 isoform X1) c4(327070, 327071, 327072) c5(327073, 327074, 327075, 327076, 327077, 327078, 327079, 327080, 327081, 327082, 327083, 327084, 327085, 327086, 327087, 327088, 327089, 327090) c6( ) c7(T, Xd, Hc, aA, fr, ci, lu, zP, ad, jz, Xc, ds, dV, cy, Xb, zQ, aq, aF, g, bz, xK, m, bt, B); #c1(ANXA5) c2(NP_001145) c3(annexin A5) c4(60091) c5(60092, 60093, 60094, 60095, 60096, 60097) c6( ) c7(T, Js, ea, fd, eb, ci, x, K, aW, g, eF, uC, eY, iA, cj, k, Xf, m, pw, dD, aw, ez, n, qN, cd, jz, Xe, h, lG, dL, ey, cY, OV, A, aq, kT, qq, at, it, B, Jo, bL, jv, fM, eR, aA, j, q, eA, zn, aT, cy, Y, r, en, cl, t, iK, kP, hc, rA, bt, ao, kh, qb, jN, Jy, jh, oR, Gn, eq, gb, hX, fU, fb, d, ff, bz, es, pc, pE, ag, gZ, ei, y, iu); #c1(ANXA6) c2(NP_001146) c3 (annexin A6 isoform 1) c4( ) c5( ) c6( ) c7(bL, bM, T, hl, Xi, bB, n, gb, cd, mC, ad, eA, x, ds, aT, Xh, ae, uQ, iU, aW, Y, ff, iw, Xg, cr, eZ, aq, Xj, en, iR, ik, bE, Vy, k, fl, ym, bt); #c1(ANXA6) c2(NP_001180473) c3(annexin A6 isoform 2) c4(74153, 74154) c5(74155, 74156, 74157, 74158, 74159, 74160, 74161, 74162, 74163, 74164, 74165) c6( ) c7(bL, bM, T, hl, Xi, bB, n, gb, cd, mC, ad, eA, x, ds, aT, Xh, ae, uQ, iU, aW, Y, ff, iw, Xg, cr, eZ, aq, Xj, en, iR, ik, bE, Vy, k, fl, ym, bt); #c1(ANXA6) c2(XP_005268489) c3(annexin A6 isoform X1) c4(281842) c5(281843, 281844, 281845, 281846, 281847, 281848, 281849, 281850, 281851, 281852, 281853, 281854) c6( ) c7(bL, bM, T, hl, Xi, bB, n, gb, cd, mC, ad, eA, x, ds, aT, Xh, ae, uQ, iU, aW, Y, ff, iw, Xg, cr, eZ, aq, Xj, en, iR, iK, bE, Vy, k, fl, ym, bt); #c1(ANXA7) c2(NP_001147) c3 (annexin A7 isoform 1) c4(60105, 60106, 60107) c5( ) c6( ) c7(T, d, iU, iR, dB, aW, aT, cj, r, By, aA, aq, q, g, eA, aF, ae, B, LS, ad); #c1(ANXA7) c2(NP_004025) c3(annexin A7 isoform 2) c4(142929) c5( ) c6( ) c7(T, d, iU, iR, dB, aW, aT, cj, r, By, aA, aq, q, g, eA, aF, ae, B, LS, ad); #c1(ANXA7) c2(XP_005269795) c3(annexin A7 isoform X1) c4( ) c5(282849, 282850, 282851, 282852, 282853, 282854) c6( ) c7(T, d, iU, iR, dB, aW, aT, cj, r, By, aA, aq, q, g, eA, aF, ae, B, LS, ad); #c1(ANXA7) c2(XP_005269796) c3(annexin A7 isoform X2) c4( ) c5(282855, 282856, 282857, 282858, 282859, 282860, 282861, 282862, 282863, 282864, 282865) c6( ) c7(T, d, iU, iR, dB, aW, aT, cj, r, By, aA, aq, q, g, eA, aF, ae, B, LS, ad); #c1(ANXA8) c2(NP_001035173) c3(annexin A8 isoform 2) c4(32545) c5(32546, 32547, 32548, 32549, 32550, 32551, 32552, 32553, 32554, 32555) c6( ) c7(ck, T, aE, ff, jh, aT, ci, Xk, en, g, hF, bt, kS, jj); #c1(ANXA8) c2(NP_001258632) c3(annexin A8 isoform 3) c4(96228, 96229) c5(96230, 96231, 96232, 96233, 96234, 96235, 96236, 96237, 96238, 96239, 96240, 96241, 96242, 96243, 96244, 96245) c6( ) c7(ck, T, aE, ff, jh, aT, ci, Xk, en, g, hF, bt, kS, j); #c1(ANXA8) c2(XP_011538398) c3(annexin A8 isoform X5) c4(336062) c5(336063, 336064, 336065, 336066) c6( ) c7(ck, T, aE, ff, jh, aT, ci, Xk, en, g, hF, bt, kS, jj); #c1(ANXA8) c2(XP_011538399) c3(annexin A8 isoform X6) c4( ) c5(336067) c6( ) c7(ck, T, aE, ff, jh, aT, ci, Xk, en, g, hF, bt, kS, jj); #c1(ANXA8) c2(XP_011538402) c3(annexin A8 isoform X9) c4( ) c5(336068) c6( ) c7(ck, T, aE, ff, jh, aT, ci, Xk, en, g, hF, bt, kS, jj); #c1(ANXA8) c2(XP_011538403) c3(annexin A8 isoform X11) c4(336069, 336070) c5(336071, 336072, 336073, 336074, 336075, 336076, 336077, 336078, 336079, 336080, 336081, 336082, 336083, 336084, 336085) c6( ) c7(ck, T, aE, ff, jh, aT, ci, Xk, en, g, hF, bt, kS, jj); #c1(ANXA8L1) c2(NP_001092315) c3(annexin A8-like protein 1 isoform 1) c4(39550, 39551) c5(39552, 39553, 39554, 39555, 39556) c6( ) c7(ck, T, aE, ff, jh, ci, Xk, en, g, hF, bt, kS, jj); #c1(ANXA8L1) c2(NP_001265853) c3(annexin A8-like protein 1 isoform 2) c4(99492, 99493, 99494) c5(99495, 99496, 99497, 99498, 99499, 99500, 99501, 99502, 99503, 99504, 99505, 99506, 99507, 99508, 99509, 99510, 99511, 99512) c6( ) c7(ck, T, aE, ff, jh, ci, Xk, en, g, hF, bt, kS, jj); #c1(ADAH) c2(NP_001170977) c3 (acyloxyacyl hydrolase isoform 2 preproprotein) c4(69620, 69621) c5(69622, 69623, 69624, 69625, 69626, 69627, 69628, 69629, 69630, 69631, 69632, 69633, 69634) c6( ) c7(bM, oe, cz, ey, C, an, xa); #c1(ADAH) c2(NP_001170978) c3(acyloxyacyl hydrolase isoform 3 preproprotein) c4(69635) c5(69636, 69637, 69638, 69639, 69640, 69641, 69642, 69643, 69644, 69645, 69646, 69647) c6( ) c7(bM, oe, cz, ey, C, an, xa); #c1(ADAH) c2(NP_001628) c3(acyloxyacyl hydrolase isoform 1 preproprotein) c4(119352, 119353) c5(119354, 119355, 119356, 119357, (19358, 119359) c6( ) c7(bM, oe, cz, ey, C, an, xa); #c1(ADC1) c2(XP_011514310) c3(amiloride-sensitive amine oxidase [copper-containing] isoform X2) c4(306999, 307000, 307001) c5(307002, 307003, 307004, 307005, 307006, 307007) c6( ) c7(bM, T, EQ, cz, ti, Fo, aT, dL, bK, an, bq, oi, rz, XI, eL, Nr, aW, Xm, iV, Nq, cT, JM); #c1(AOC1) c2(XP_011514311) c3(amiloride-sensitive amine oxidase [copper-containing] isoform X1) c4(307008, 307009, 307010) c5(307011, 307012, 307013, 307014, 307015, 307016, 307017, 307018, 307019, 307020, 307021, 307022, 307023, 307024, 307025, 307026, 307027, 307028, 307029, 307030, 307031, 307032, 307033, 307034, 307035, 307036, 307037, 307038, 307039, 307040, 307041, 307042, 307043, 307044, 307045, 307046, 307047, 307048) c6( ) c7(bM, T, EQ, cz, ti, Fo, aT, dL, bk, an, bq, oi, rz, XI, eL, Nr, aW, Xm, iV, Nq, cT, JM); #c1(ADC2) c2(NP_001149) c3(retina-specific copper amine oxidase isoform a precursor) c4( ) c5( ) c6( ) c7(bL, A, oz, cz, dq, aj, aS, fD, J); #c1(ADC2) c2(NP_033720) c3(retina-specific copper amine oxidase isoform b precursor) c4(172316) c5(172317, 172318, 172319, 172320, 172321, 172322) c6( ) c7(bL, A, oz, cz, dq, aj, aS, fD, J); #c1(ADC3) c2(NP_003725) c3(membrane primary amine oxidase isoform 1) c4(140465) c5(140466, 140467, 140468, 140469, 140470, 140471) c6( ) c7(bL, cz, wo, aj, v, qW, ai, J, oz, lG, bq, vx, uX, DI, A, lx, bf, Y, fO, hl, dq, W, WP, aS, dy, fD, GF, us); #c1(AOX1) c2(NP_001150) c3(aldehyde oxidase) c4(60108) c5(60109, 60110, 60111, 60112, 60113, 60114, 60115, 60116, 60117, 60118, 60119, 60120) c6( ) c7(Xn, fe, dq, ci, ad, bA); #c1(AOX1) c2(XP_011509364) c3(aldehyde oxidase isoform X1) c4(299953) c5( ) c6( ) c7(Xn, fe, dq, ci, ad, bA); #c1(AP1AR) c2(NP_001121898) c3(AP-1 complex-associated regulatory protein isoform b) c4(46279) c5(46280, 46281, 46282, 46283, 46284, 46285) c6( ) c7(ey); #c1(AP1AR) c2(NP_061039) c3(AP-1 complex-associated regulatory protein isoform a) c4(199097) c5(199098, 199099, 199100, 199101, 199102, 199103) c6( ) c7(ey); #c1(AP1B1) c2(NP_001159491) c3(AP-1 complex subunit beta-1 isoform c) c4(64/70) c5(64171, 64/72, 64173, 64174, 64175, 64/76) c6( ) c7(jx, ip); #c1(AP1B1) c2(NP_663782) c3(AP-1 complex subunit beta-1 isoform b) c4(241562, 241563) c5(241564, 241565, 241566, 241567, 241568, 241569, 241570, 241571, 241572, 241573, 241574, 241575, 241576, 241577) c6( ) c7(jx, ip); #c1(AP1G1) c2(NP_001025178) c3(AP-1 complex subunit gamma-1 isoform a) c4(29430, 29431, 29432, 29433) c5(29434, 29435, 29436, 29437, 29438, 29439, 29440, 29441, 29442, 29443, 29444, 29445, 29446, 29447, 29448, 29449, 29450, 29451) c6( ) c7(vi); #c1(AP1M1) c2(NP_001123996) c3(AP-1 complex subunit mu-l isoform 1) c4(48221) c5(48222, 48223, 48224, 48225, 48226, 48227) c6( ) c7(an); #c1(AP1M1) c2(NP_115882) c3(AP-1 complex subunit mu-l isoform 2) c4(224464, 224465, 224466) c5(224467, 224468, 224469, 224470, 224471, 224472, 224473, 224474, 224475, 224476, 224477, 224478) c6( ) c7(an); #c1(AP1M1) c2(XP_011526702) c3(AP-1 complex subunit mu-1 isoform X1) c4( ) c5(321015, 321016, 321017, 321018, 321019, 321020) c6( ) c7(an); #c1(AP1M2) c2(NP_005489) c3(AP-1 complex subunit mu-2 isoform 2) c4(156745) c5(156746, 156747, 156748, 156749, 156750, 156751) c6( ) c7(Uc, w, fr); #c1(AP1S1) c2(NP_001274) c3(AP-1 complex subunit sigma-(A) c4(106780) c5( ) c6( ) c7(du, Xp, nA, Xa); #c1(AP1S2) c2(NP_001259000) c3(AP-1 complex subunit sigma-2 isoform 1) c4( ) c5(96914, 96915, 96916, 96917, 96918, 96919, 96920, 96921, 96922, 96923, 96924) c6( ) c7(ul, aT, nf, Xr, nA, Xq, bN); #c1(AP1S2) c2(NP_003907) c3(AP-1 complex subunit sigma-2 isoform 2) c4(142085) c5(142086, 142087, 142088, 142089, 142090, 142091) c6( ) c7(ul, aT, nf, Xr, nA, Xq, bN); #c1(AP1S2) c2(XP_005274671) c3(AP-1 complex subunit sigma-2 isoform X2) c4(285314, 285315, 285316) c5(285317, 285318, 285319, 285320, 285321, 285322, 285323, 285324, 285325, 285326, 285327, 285328, 285329) c6( ) c7(ul, aT, nf, Xr, nA, Xq, bN); #c1(AP1S2) c2(XP_011543901) c3(AP-1 complex subunit sigma-2 isoform X1) c4(343077, 343078, 343079) c5(343080, 343081, 343082, 343083, 343084, 343085, 343086, 343087, 343088, 343089, 343090, 343091) c6( ) c7(ul, aT, nf, Xr, nA, Xq, bN); #c1(AP1S3) c2(NP_001034658) c3(AP-1 complex subunit sigma-3) c4(31983, 31984, 31985) c5(31986, 31987, 31988, 31989, 31990, 31991, 31992, 31993, 31994, 31995, 31996, 31997, 31998, 31999, 32000, 32001, 32002, 32003) c6( ) c7(Xs, aw, lQ); #c1(AP1S3) c2(XP_011508902) c3(AP-1 complex subunit sigma-3 isoform X1) c4(299476) c5(299477, 299478, 299479, 299480, 299481, 299482) c6( ) c7(Xs, aw, lQ); #c1(AP2A1) c2(NP_055018) c3(AP-2 complex subunit alpha-1 isoform 1) c4(178320) c5(178321, 178322, 178323, 178324, 178325, 178326) c6( ) c7(bv, T, aE, JI, aW, Y, ff, aT, dN, kp, ci, en, Xt, bt); #c1(AP2A1) c2(NP_570603) c3(AP-2 complex subunit alpha-l isoform 2) c4(234083) c5(234084, 234085, 234086, 234087, 234088, 234089) c6( ) c7(bv, T, aE, JI, aW, Y, ff, aT, dN, kp, ci, en, Xt, bt); #c1(AP2B1) c2(NP_001025177) c3(AP-2 complex subunit beta isoform a) c4(29412, 29413) c5(29414, 29415, 29416, 29417, 29418, 29419, 29420, 29421, 29422, 29423, 29424, 29425, 29426, 29427, 29428, 29429) c6( ) c7(gf, bS, ct, Xu, Z, ip); #c1(AP2MI) c2(NP_001020376) c3(AP-2 complex subunit mu isoform b) c4(28693, 28694) c5(28695, 28696, 28697, 28698, 28699, 28700, 28701, 28702, 28703, 28704, 28705, 28706, 28707, 28708, 28709, 28710, 28711, 28712, 28713, 28714, 28715, 28716) c6( ) c7(eR, Qu, ff, jx, wV, en); #c1(AP2M1) c2(NP_004059) c3(AP-2 complex subunit mu isoform a) c4(143208, 143209, 143210) c5(143211, 143212, 143213, 143214, 143215, 143216, 143217, 143218, 143219, 143220, 143221, 143222) c6( ) c7(eR, Qu, ff, jx, wV, en); #c1(APZS1) c2(NP_004060) c3(AP-2 complex subunit sigma isoform AP17) c4(143223) c5(143224, 143225, 143226, 143227, 143228, 143229) c6( ) c7(Xw, OX, TG, HI, Xv); #c1(AP2S1) c2(NP_067586) c3(AP-2 complex subunit sigma isoform AP17delta) c4(209034) c5(209035, 209036, 209037, 209038, 209039, 209040, 209041, 209042, 209043, 209044) c6( ) c7(Xw, OX, TG, HI, Xv); #c1(AP3B1) c2(NP_001258698) c3(AP-3 complex subunit beta-1 isoform 2) c4(96368) c5(96369, 96370, 96371, 96372, 96373, 96374) c6( ) c7(gV, fG, e, aw, Xx, j, pz, q, XB, TI, dY, XD, bW, h, eu, aa, lE, hG, XA, oV, Y, XE, fc, XC, r, aq, Xz, Xy, KM, ac, hk, lF, cP, B); #c1(AP3BI) c2(NP_003655) c3(AP-3 complex subunit beta-1 isoform 1) c4(139995) c5(139996, 139997, 139998, 139999, 140000, 140001, 140002, 140003, 140004, 140005, 140006, 140007) c6( ) c7(gV, fG, e, aw, Xx, j, pz, q, XB, TI, dY, XD, bW, h, eu, aa, lE, hG, XA, oV, Y, XE, fc, XC, r, aq, Xz, Xy, KM, ac, hk, lF, cP, B); #c1(AP3B1) c2(XP_005248676) c3(AP-3 complex subunit beta-1 isoform X2) c4(268511) c5( ) c6( ) c7(gV, fG, e, aw, Xx, j, pz, q, XB, TI, dY, XD, bW, h, eu, aa, lE, hG, XA, oV, Y, XE, fc, XC, r, aq, Xz, Xy, KM, ac, hk, lF, cP, B); #c1(AP3D1) c2(NP_001248755) c3(AP-3 complex subunit delta-l isoform 3) c4(93995, 93996) c5(93997, 93998, 93999, 94000, 94001, 94002, 94003, 94004, 94005, 94006, 94007, 94008) c6( ) c7(IE, ac, fR, XF, lF, Xx, fq); #c1(AP3D1) c2(NP_003929) c3(AP-3 complex subunit delta-1 isoform 2) c4(142240, 142241) c5(142242, 142243, 142244, 142245, 142246, 142247, 142248, 142249, 142250, 142251, 142252, 142253) c6( ) c7(IE, ac, fR, XF, lF, Xx, fq); #c1(AP3DI) c2(XP_006722995) c3(AP-3 complex subunit delta-1 isoform X1) c4(294957) c5(294958, 294959, 294960, 294961, 294962, 294963) c6( ) c7(IE, ac, fR, XF, lF, Xx, fq); #c1(AP3M2) c2(NP_006794) c3(AP-3 complex subunit mu-2) c4(168107, 168108, 168109) c5(168110, 168111, 168112, 168113, 168114, 168115, 168116, 168117, 168118, 168119, 168120, 168121, 168122, 168123, 168124, 168125, 168126, 168127, 168128, 168129, 168130, 168131, 168132, 168133, 168134, 168135, 168136, 168137, 168138, 168139) c6( ) c7(ha); #c1(AP3SI) c2(NP_001275) c3(AP-3 complex subunit sigma-1) c4(107711) c5( ) c6( ) c7(bL); #c1(AP3S2) c2(NP_005820) c3(AP-3 complex subunit sigma-2) c4(159681, 159682) c5(159683, 159684, 159685, 159686, 159687, 159688, 159689, 159690, 159691, 159692, 159693, 159694, 159695, 159696, 159697, 159698) c6( ) c7(bL, dY); #c1(AP4B1) c2(XP_011538826) c3(AP-4 complex subunit beta-1 isoform X1) c4(336792) c5(336793, 336794, 336795, 336796, 336797, 336798) c6( ) c7(sc, T, e, ha, jN, fN, XJ, jh, n, ci, nA, XM, XH, Fc, br, ft, aE, lg, aT, dL, K, ae, eL, hM, Jg, XK, d, A, aW, ff, lo, en, g, cm, XI, bz, pb, dr, bw, es, XG, XO, XN, X, XL, L); #c1(AP4B1) c2(XP_011538827) c3(AP-4 complex subunit beta-1 isoform X2) c4(336799) c5(336800, 336801, 336802, 336803, 336804, 336805) c6( ) c7(sc, T, e, ha, N, fN, XJ, jh, n, ci, nA, XM, XH, Fc, br, ft, aE, lg, aT, dL, K, ae, eL, hM, Jg, XK, d, A, aW, ff, lo, en, g, cm, XI, bz, pb, dr, bw, es, XG, XO, XN, X, XL, L); #c1(AP4E1) c2(NP_031373) c3(AP-4 complex subunit epsilon-1 isoform 1) c4(172031) c5(172032, 172033, 172034, 172035, 172036, 172037) c6( ) c7(WI, oR, XP, aV, nA); #c1(AP4MI) c2(NP_004713) c3(AP-4 complex subunit mu-1) c4(149011) c5(149012, 149013, 149014, 149015, 149016, 149017, 149018, 149019, 149020, 149021) c6( ) c7(WI, oR, XQ, nA); #c1(AP4SI) c2(NP_001121598) c3(AP-4 complex subunit sigma-l isoform 2) c4(45804, 45805, 45806) c5(45807, 45808, 45809, 45810, 45811, 45812, 45813, 45814, 45815, 45816, 45817, 45818, 45819, 45820, 45821, 45822, 45823, 45824) c6( ) c7(XR); #c1(AP4SI) c2(NP_001241655) c3(AP-4 complex subunit sigma-1 isoform 3) c4(88980) c5(88981, 88982, 88983, 88984, 88985, 88986, 88987, 88988, 88989, 88990, 88991, 88992) c6( ) c7(XR); #c1(AP4SI) c2(NP_001241656) c3(AP-4 complex subunit sigma-l isoform 4) c4(88993) c5(88994, 88995, 88996, 88997, 88998, 88999) c6( ) c7(XR); #c1(AP4SI) c2(NP_009008) c3(AP-4 complex subunit sigma-1 isoform 1) c4(170214, 170215) c5(170216, 170217, 170218, 170219, 170220, 170221) c6( ) c7(XR); #c1(AP5MI) c2(NP_060699) c3(AP-5 complex subunit mu-1) c4(197286) c5(197287, 197288, 197289, 197290, 197291, 197292) c6( ) c7(hF, bt, fX); #c1(AP5Z1) c2(NP_055670) c3(AP-5 complex subunit zeta-1) c4(182634, 182635) c5(182636, 182637, 182638, 182639, 182640, 182641, 182642, 182643, 182644, 182645, 182646, 182647) c6( ) c7(x, T, d, qe, aV, XS, eZ, k, ER, as, Fc); #c1(APAF1) c2(NP_001151) c3(apoptotic protease-activating factor 1 isoform b) c4(60128) c5( ) c6( ) c7(T, id, jx, bO, aA, bB, n, fd, jh, fr, ci, zP, hF, q, ad, eA, XX, XW, x, py, lG, dL, bl, K, XU, pk, ae, eL, eu, cp, d, lx, s, vT, iU, Y, eH, Az, XV, r, g, eF, t, cm, bz, XT, vk, ck, es, Dy, eM, iA, dB, Ip, bE, iT, U, cj, at, oh, k, m, bt); #c1(APAF1) c2(NP_037361) c3(apoptotic protease-activating factor 1 isoform a) c4(175005) c5(175006, 175007, 175008, 175009, 175010, 175011) c6( ) c7(T, id, jx, bO, aA, bB, n, fd, jh, fr, ci, zP, hF, q, ad, eA, XX, XW, x, py, lG, dL, bl, K, XU, pk, ae, el, eu, cp, d, lx, s, vT, iU, Y, eH, Az, XV, r, g, eF, t, cm, bz, XT, vk, ck, es, Dy, eM, iA, dB, lp, bE, iT, U, cj, at, oh, k, m, bt); #c1(APAF1) c2(NP_863651) c3(apoptotic protease-activating factor 1 isoform c) c4(255830) c5(255831, 255832, 255833, 255834, 255835, 255836) c6( ) c7(T, id, jx, bO, aA, bB, n, fd, jh, fr, ci, zP, hF, q, ad, eA, XX, XW, x, py, lG, dL, bl, K, XU, pk, ae, eL, eu, cp, d, lx, s, vT, iU, Y, eH, Az, XV, r, g, eF, t, cm, bz, XT, vk, ck, es, Dy, eM, iA, dB, lp, bE, iT, U, cj, at, oh, k, m, bt); #c1(APAF1) c2(NP_863658) c3 (apoptotic protease-activating factor 1 isoform d) c4(255837, 255838) c5(255839, 255840, 255841, 255842, 255843, 255844, 255845, 255846, 255847, 255848, 255849, 255850, 255851, 255852, 255853, 255854, 255855, 255856, 255857, 255858) c6( ) c7(T, id, jx, bO, aA, bB, n, fd, jh, fr, ci, zP, hF, q, ad, eA, XX, XW, x, py, lG, dL, bl, K, XU, pk, ae, eL, eu, cp, d, lx, s, vT, iU, Y, eH, Az, XV, r, g, eF, t, cm, bz, XT, vk, ck, es, Dy, eM, iA, dB, lp, bE, iT, U, cj, at, oh, k, m, bt); #c1(APAF1) c2(NP_863659) c3(apoptotic protease-activating factor 1 isoform e) c4(255859) c5(255860, 255861, 255862, 255863, 255864, 255865, 255866, 255867, 255868, 255869) c6( ) c7(T, id, jx, bO, aA, bB, n, fd, jh, fr, ci, zP, hF, q, ad, eA, XX, XW, x, py, lG, dL, bl, K, XU, pk, ae, eL, eu, cp, d, lx, s, vT, iU, Y, eH, Az, XV, r, g, eF, t, cm, bz, XT, vk, ck, es, Dy, eM, iA, dB, Ip, bE, iT, U, cj, at, oh, k, m, bt); #c1(APBA1) c2(XP_011516919) c3(amyloid beta A4 precursor protein-binding family A member 1 isoform X1) c4(310434) c5(310435, 310436, 310437, 310438, 310439, 310440) c6( ) c7(bF, s, dJ, r, q, g, iV, fb, af); #c1(APBA2) c2(XP_006725606) c3(amyloid beta A4 precursor protein-binding family A member 2 isoform X1) c4(296914) c5(296915, 296916, 296917, 296918, 296919, 296920, 296921, 296922, 296923, 296924) c6( ) c7(bF, T, s, rz, yL, ha, hE, aA, kt, uJ, aF, ci, g, H, fb, bN, L); #c1(APBA2) c2(XP_011545700) c3(amyloid beta A4 precursor protein-binding family A member 2 isoform X2) c4(344613, 344614, 344615, 344616) c5(344617, 344618, 344619, 344620, 344621, 344622, 344623, 344624, 344625, 344626, 344627, 344628, 344629, 344630, 344631, 344632, 344633, 344634, 344635, 344636, 344637, 344638, 344639, 344640) c6( ) c7(bF, T, s, rz, yL, ha, hE, aA, kt, uJ, aF, ci, g, H, fb, bN, L); #c1(APBA2) c2(XP_011545702) c3(amyloid beta A4 precursor protein-binding family A member 2 isoform X4) c4(344641, 344642) c5(344643, 344644, 344645, 344646, 344647, 344648, 344649, 344650, 344651, 344652, 344653, 344654, 344655, 344656, 344657, 344658, 344659, 344660, 344661) c6( ) c7(bF, T, s, rz, yL, ha, hE, aA, kt, uJ, aF, ci, g, H, fb, bN, L); #c1(APBA3) c2(NP_004877) c3(amyloid beta A4 precursor protein-binding family A member 3) c4(150488) c5(150489, 150490, 150491, 150492, 150493, 150494) c6( ) c7(cF, af); #c1(APBB1) c2(NP_001244248) c3(amyloid beta A4 precursor protein-binding family B member 1 isoform g) c4( ) c5(92060) c6( ) c7(aW, XY, lb, U, aT, kS, af, L); #c1(APBB1) c2(NP_001244252) c3(amyloid beta A4 precursor protein-binding family B member 1 isoform f) c4( ) c5(92061, 92062, 92063, 92064, 92065, 92066) c6( ) c7(aW, XY, lb, U, aT, kS, af, L); #c1(APBB1) c2(NP_001244254) c3(amyloid beta A4 precursor protein-binding family B member 1 isoform e) c4(92067) c5(92068, 92069, 92070, 92071, 92072, 92073) c6( ) c7(aW, XY, lb, U, aT, kS, af, L); #c1(APBB1) c2(NP_663722) c3(amyloid beta A4 precursor protein-binding family B member 1 isoform b) c4(241445) c5(241446, 241447, 241448, 241449, 241450, 241451, 241452, 241453, 241454, 241455) c6( ) c7(aW, XY, lb, U, aT, kS, af, L); #c1(APBB1) c2(XP_006718277) c3(amyloid beta A4 precursor protein-binding family B member 1 isoform X6) c4(291653) c5( ) c6( ) c7(aW, XY, lb, U, aT, kS, af, L); #c1(APBB1) c2(XP_006718278) c3(amyloid beta A4 precursor protein-binding family B member 1 isoform X1) c4(291654, 291655, 291656) c5(291657, 291658, 291659, 291660, 291661, 291662, 291663, 291664, 291665, 291666, 291667, 291668) c6( ) c7(aW, XY, lb, U, aT, kS, af, L); #c1(APBB1) c2(XP_011518343) c3(amyloid beta A4 precursor protein-binding family B member 1 isoform X2) c4( ) c5(312344, 312345, 312346, 312347, 312348) c6( ) c7(aW, XY, lb, U, aT, kS, af, L); #c1(APBB1IP) c2(XP_0067/7514) c3(amyloid beta A4 precursor protein-binding family B member 1-interacting protein isoform X1) c4(291161, 291162) c5(291163, 29/164, 291165, 291166, 291167, 291168, 291169, 291170, 291171, 291172, 291173, 291174) c6( ) c7(dA, W, Y, ae, af, ad); #c1(APBB1IP) c2(XP_011517816) c3(amyloid beta A4 precursor protein-binding family B member 1-interacting protein isoform X2) c4(311753) c5(311754, 311755, 311756, 311757, 311758, 311759) c6( ) c7(dA, W, Y, ae, af, ad); #c1(APBB2) c2(NP_001159522) c3(amyloid beta A4 precursor protein-binding family B member 2 isoform b) c4(64203, 64204) c5(64205, 64206, 64207, 64208, 64209, 64210, 64211, 64212, 64213, 64214, 64215, 64216) c6( ) c7(cF, fK, af, L); #c1(APBB2) c2(NP_001159525) c3(amyloid beta A4 precursor protein-binding family B member 2 isoform d) c4( ) c5(64217, 64218, 64219, 64220, 64221, 64222, 64223, 64224, 64225, 64226, 64227) c6( ) c7(cF, fK, af, L); #c1(APBB2) c2(NP_004298) c3(amyloid beta A4 precursor protein-binding family B member 2 isoform a) c4(144966) c5(144967, 144968, 144969, 144970, 144971, 144972) c6( ) c7(cF, fK, af, L); #c1(APBB2) c2(NP_775098) c3(amyloid beta A4 precursor protein-binding family B member 2 isoform c) c4(249501) c5(249502, 249503, 249504, 249505, 249506, 249507) c6( ) c7(cF, fK, af, L); #c1(APBB2) c2(XP_005248158) c3(amyloid beta A4 precursor protein-binding family B member 2 isoform X6) c4(268255, 268256) c5(268257, 268258, 268259, 268260, 268261, 268262, 268263) c6( ) c7(cF, fK, af, L); #c1(APBB2) c2(XP_006714072) c3(amyloid beta A4 precursor protein-binding family B member 2 isoform X1) c4( ) c5(287526, 287527, 287528, 287529, 287530, 287531, 287532, 287533, 287534) c6( ) c7(cF, fK, af, L); #c1(APBB2) c2(XP_006714073) c3(amyloid beta A4 precursor protein-binding family B member 2 isoform X2) c4(287535) c5( ) c6( ) c7(cF, fK, af, L); #c1(APBB2) c2(XP_006714075) c3(amyloid beta A4 precursor protein-binding family B member 2 isoform X4) c4( ) c5(287536, 287537) c6( ) c7(cF, fK, af, L); #c1(APBB2) c2(XP_011511989) c3(amyloid beta A4 precursor protein-binding family B member 2 isoform X5) c4(302656) c5(302657, 302658, 302659, 302660, 302661, 302662) c6( ) c7(cF, fK, af, L); #c1(APBB2) c2(XP_011511990) c3(amyloid beta A4 precursor protein-binding family B member 2 isoform X7) c4(302663) c5(302664, 302665, 302666, 302667, 302668, 302669, 302670) c6( ) c7(cF, fK, af, L); #c1(APBB3) c2(NP_006042) c3(amyloid beta A4 precursor protein-binding family B member 3 isoform d) c4(161418, 161419) c5(161420, 161421, 161422, 161423, 161424, 161425, 161426, 161427, 161428, 161429, 161430, 161431, 161432) c6( ) c7(aW, aT, aq, mf, XZ, ae, af, ad); #c1(APBB3) c2(NP_573418) c3(amyloid beta A4 precursor protein-binding family B member 3 isoform c) c4(234497, 234498, 234499) c5(234500, 234501, 234502, 234503, 234504, 234505) c6( ) c7(aW, aT, aq, mf, XZ, ae, af, ad); #c1(APBB3) c2(NP_573419) c3(amyloid beta A4 precursor protein-binding family B member 3 isoform b) c4(234506, 234507) c5(234508, 234509, 234510, 234511, 234512, 234513, 234514, 234515, 234516, 234517, 234518, 234519, 234520) c6( ) c7(aW, aT, aq, mf, XZ, ae, af, ad); #c1(APBB3) c2(NP_573420) c3(amyloid beta A4 precursor protein-binding family B member 3 isoform a) c4(234521) c5(234522, 234523, 234524, 234525, 234526, 234527, 234528, 234529, 234530, 234531, 234532, 234533, 234534, 234535, 234536, 234537, 234538) c6( ) c7(aW, aT, aq, mf, XZ, ae, af, ad); #c1(APC) c2(NP_001120982) c3(adenomatous polyposis coli protein isoform b) c4(45049, 45050, 45051, 45052) c5(45053, 45054, 45055, 45056, 45057, 45058, 45059, 45060, 45061, 45062, 45063, 45064, 45065, 45066, 45067, 45068, 45069, 45070, 45071, 45072, 45073, 45074, 45075, 45076, 45077, 45078, 45079, 45080, 45081, 45082, 45083, 45084) c6( ) c7(uB, T, aC, Fj, pf, YU, if, al, Yb, bB, ci, QG, YD, ip, aE, pX, Yy, Yk, iv, gs, K, hu, dQ, eL, DQ, Ys, YL, yo, zQ, JI, aW, YC, nY, LZ, YG, Yp, NR, g, eF, zE, bw, iP, qJ, y, fT, eM, nB, AM, cj, iS, YP, m, YS, YK, cE, P, Qj, iB, ht, NT, fN, n, aj, fr, nA, qN, eh, eW, BI, ft, no, jz, Yc, lt, By, dL, he, KV, YT, pl, YI, YO, A, MD, sq, Yd, iw, ge, fe, aq, YF, Yr, hq, Ye, Yf, YI, iO, YE, bP, dB, se, hk, hL, at, pg, YH, it, B, YJ, YN, aA, cR, j, C, AO, q, J, Yh, YA, er, eJ, Yn, Yv, nX, yg, aT, yu, rz, ae, jf, Uf, Yw, s, vT, Ya, Y, NE, r, lo, en, Yx, cl, t, Ym, bN, YW, Yq, Dy, ac, VJ, nM, iM, Li, au, YV, gX, Yz, LS, VB, hS, Mb, hl, Yu, df, YM, ml, jh, KO, Fc, iJ, ad, hX, av, Yt, hd, ib, NS, Yo, qV, lC, YB, ji, YQ, fb, nJ, iL, Yi, WW, MG, Yj, d, je, BO, qt, Fm, ff, xs, eH, Kf, hp, aF, iV, bz, dr, YR, bF, es, gk, bE, ag, AR, pP, Yg, MC, HE, X, jr); #c1(APC2) c2(XP_005259532) c3(adenomatous polyposis coli protein 2 isoform X3) c4(276121) c5(276122, 276123) c6( ) c7(bF, T, bE, aT, bB, Hn, fe, K, m, g, t, iu); #c1(APC2) c2(XP_006722672) c3(adenomatous polyposis coli protein 2 isoform X1) c4( ) c5(294797, 294798, 294799, 294800, 294801, 294802, 294803, 294804, 294805, 294806, 294807, 294808, 294809, 294810) c6( ) c7(bF, T, bE, aT, bB, Hn, fe, K, m, g, t, iu); #c1(APC2) c2(XP_006722673) c3(adenomatous polyposis coli protein 2 isoform X2) c4(294811) c5(294812) c6( ) c7(bF, T, bE, aT, bB, Hn, fe, K, m, g, t, iu); #c1(APCDD1) c2(NP_694545) c3(protein APCDD1 precursor) c4(245097, 245098) c5(245099, 245100, 245101, 245102, 245103, 245104, 245105, 245106, 245107, 245108, 245109, 245110) c6( ) c7(ck, T, ac, bE, YX, bB, oX, Df); #c1(APCDD1L) c2(NP_001291716) c3(protein APCDD1-like isoform 2) c4(116376) c5(116377, 116378, 116379, 116380, 116381, 116382) c6( ) c7(af); #c1(APCDD1L) c2(NP_699191) c3(protein APCDD1-like isoform 1 precursor) c4(245842) c5(245843, 245844, 245845, 245846, 245847, 245848) c6( ) c7(af); #c1(APCS) c2(NP_001630) c3(serum amyloid P-component precursor) c4(119382, 119383) c5(119384, 119385, 119386, 119387, 119388, 119389, 119390, 119391, 119392, 119393, 119394, 119395, 119396, 119397, 119398, 119399, 119400, 119401, 119402, 119403, 119404, 119405, 119406, 119407, 119408, 119409, 119410, 119411, 119412, 119413, 119414, 119415, 119416, 119417, 119418, 119419, 119420, 119421, 119422, 119423, 119424, 119425, 119426, 119427, 119428, 119429, 119430, 119431, 119432, 119433, 119434, 119435, 119436, 119437, 119438, 119439, 119440, 119441, 119442, 119443, 119444, 119445, 119446, 119447, 119448, 119449, 119450, 119451, 119452, 119453, 119454, 119455, 119456, 119457, 119458, 119459, 119460, 119461, 119462, 119463, 119464, 119465, 119466, 119467, 119468, 119469, 119470, 119471, 119472, 119473, 119474, 119475, 119476) c6(119477) c7(oO, T, dt, fM, dD, fR, lT, e, aw, jh, aj, v, j, eq, C, pz, Za, mC, J, cd, hX, F, ql, ds, iY, H, pk, Zd, hm, yo, YY, oV, Zc, Zf, ih, eZ, aq, lo, YZ, Zb, dr, Wt, ck, OG, Zg, hc, CZ, yH, fl, Lz, R, pJ, Ze, B, L); #c1(APEH) c2(NP_001631) c3(acylamino-acid-releasing enzyme) c4(119478) c5(119479, 119480, 119481, 119482, 119483, 119484) c6( ) c7(T, ft, nB, aT, Zh, fr, iV, cm, bz); #c1(APEX1) c2(XP_005267638) c3(DNA-(apurinic or apyrimidinic site) lyase isoform X1) c4(281136, 281137, 281138, 281139, 281140) c5(281141, 281142, 281143, 281144, 281145, 281146, 281147, 281148, 281149, 281150, 281151, 281152, 281153, 281154, 281155, 281156, 281157, 281158, 281159, 281160, 281161, 281162, 281163, 281164, 281165, 281166, 281167, 281168, 281169, 281170, 281171, 281172, 281173, 281174, 281175, 281176, 281177, 281178) c6( ) c7(T, aC, fR, Zq, ea, if, fd, bB, Vd, ci, qj, ip, aE, u, Bc, Qk, K, hu, eL, bc, Zm, aa, JI, aW, az, hl, nY, Zr, uY, La, g, eF, uC, fT, bb, eM, nB, iT, wE, vA, U, Zn, m, Xu, Vj, Zo, dD, gU, wu, sw, aw, ht, n, fr, qN, h, lG, eV, dL, Gw, Lh, aq, Zj, op, Sc, bP, dB, hk, hl, at, el, cB, B, it, eX, aA, sM, cz, hy, j, il, Zk, V, q, eA, Zp, hW, aT, ae, af, eu, bA, Sh, s, vT, Or, Y, Lf, RF, r, en, bu, cl, t, iK, ck, rR, VJ, Dy, c, ac, dy, JH, fD, bt, Kd, GI, ZI, aD, eq, ad, dP, av, l, Yo, hD, rN, wm, NM, d, Fm, ff, eH, aF, iV, iR, bz, qa, bF, Zi, bE, gZ, Gu, Pk, iu, pC); #c1(APEX2) c2(NP_055296) c3(DNA-(apurinic or apyrimidinic site) lyase 2 isoform 1) c4(180346) c5(180347, 180348, 180349, 180350, 180351, 180352) c6( ) c7(f, V, U); #c1(APH1B) c2(NP_001139118) c3(gamma-secretase subunit APH-1B isoform 2) c4(58712, 58713) c5(58714, 58715, 58716, 58717, 58718, 58719, 58720, 58721, 58722, 58723, 58724) c6( ) c7(cW, j, af, h); #c1(APH1B) c2(NP_112591) c3(gamma-secretase subunit APH-IB isoform 1) c4(221518) c5(221519, 221520, 221521, 221522, 221523, 221524, 221525) c6( ) c7(cW, j, af, h); #c1(API5) c2(NP_001136402) c3(apoptosis inhibitor 5 isoform a) c4(54880, 54881) c5(54882, 54883, 54884, 54885, 54886, 54887, 54888, 54889, 54890, 54891, 54892, 54893) c6( ) c7(T, eM, iR, if, ff, eZ, aq, qN, en, g, B); #c1(API5) c2(NP_001136403) c3(apoptosis inhibitor 5 isoform c) c4(54894) c5(54895, 54896, 54897, 54898, 54899, 54900) c6( ) c7(T, eM, iR, if, ff, eZ, aq, qN, en, g, B); #c1(API5) c2(NP_006586) c3(apoptosis inhibitor 5 isoform b) c4(166418) c5(166419, 166420, 166421, 166422, 166423, 166424) c6( ) c7(T, eM, iR, if, ff, eZ, aq, qN, en, g, B); #c1(APIP) c2(NP_057041) c3(methylthioribulose-1-phosphate dehydratase) c4(188606) c5(188607, 188608, 188609, 188610, 188611, 188612) c6( ) c7(T, oe, eM); #c1(APIP) c2(XP_011518456) c3(methylthioribulose-I-phosphate dehydratase isoform X1) c4(312525) c5(312526, 312527, 312528, 312529, 312530, 312531) c6( ) c7(T, oe, eM); #c1(APLN) c2(NP_059109) c3(apelin preproprotein) c4(193055) c5(193056, 193057, 193058, 193059, 193060, 193061, 193062, 193063) c6( ) c7(bL, T, dB, al, mx, cz, aj, v, j, ci, g, eA, ji, uC, cd, J); #c1(APLNR) c2(NP_005152) c3(apelin receptor) c4(153444, 153445, 153446) c5(153447, 153448, 153449, 153450, 153451, 153452, 153453, 153454, 153455, 153456, 153457, 153458, 153459, 153460, 153461, 153462, 153463, 153464, 153465) c6( ) c7(bL, dD, ea, cz, aj, v, j, eq, cd, J, eA, Zs, JD, h, K, vx, cY, mo, uC, qa, wg, ac, dB, mx, mg, eo, lC, vQ); #c1(APLP1) c2(NP_001019978) c3(amyloid-like protein 1 isoform 1 precursor) c4(27972) c5(27973, 27974, 27975, 27976, 27977, 27978) c6( ) c7(vW, pX, U, cm, K, H, Yi, L); #c1(APLP1) c2(NP_005157) c3(amyloid-like protein 1 isoform 2 precursor) c4(153484) c5(153485, 153486, 153487, 153488, 153489, 153490) c6( ) c7(vW, pX, U, cm, K, H, Yi, L); #c1(APLP2) c2(NP_001135748) c3(amyloid-like protein 2 isoform 2 precursor) c4(53026, 53027) c5(53028, 53029, 53030, 53031, 53032, 53033, 53034, 53035, 53036, 53037, 53038) c6( ) c7(dx, T, d, aE, el, fc, rU, HC, cm, QL, j, K, cP, qF, eA, H, af, Yi, L); #c1(APLP2) c2(NP_001135749) c3(amyloid-like protein 2 isoform 3 precursor) c4(53039, 53040, 53041, 53042) c5(53043, 53044, 53045, 53046, 53047, 53048, 53049, 53050, 53051, 53052, 53053, 53054) c6( ) c7(dx, T, d, aE, el, fc, ru, HC, cm, QL, j, K, cP, qF, eA, H, af, Yi, L); #c1(APLP2) c2(NP_001135750) c3(amyloid-like protein 2 isoform 4 precursor) c4(53055) c5(53056, 53057, 53058, 53059, 53060, 53061, 53062, 53063, 53064, 53065, 53066, 53067, 53068, 53069, 53070, 53071, 53072, 53073) c6( ) c7(dx, T, d, aE, el, fc, rU, HC, cm, QL, j, K, cP, qF, eA, H, af, Yi, L); #c1(APLP2) c2(NP_001230228) c3(amyloid-like protein 2 isoform 5) c4(86137) c5(86138, 86139, 86140, 86141, 86142, 86143) c6( ) c7(dx, T, d, aE, el, fc, rU, HC, cm, QL, j, K, cP, qF, eA, H, af, Yi, L); #c1(APLP2) c2(NP_001633) c3(amyloid-like protein 2 isoform 1 precursor) c4(119485) c5(119486, 119487, 119488, 119489, 119490, 119491) c6( ) c7(dx, T, d, aE, el, fc, rU, HC, cm, QL, j, K, cP, qF, eA, H, af, Yi, L); #c1(APOA1BP) c2(NP_658985) c3(NAD(P)H-hydrate epimerase precursor) c4(239620, 239621, 239622, 239623) c5(239624, 239625, 239626, 239627, 239628, 239629, 239630, 239631, 239632, 239633, 239634, 239635, 239636, 239637, 239638, 239639, 239640, 239641, 239642, 239643, 239644, 239645, 239646, 239647, 239648, 239649, 239650, 239651, 239652, 239653, 239654, 239655) c6(239656, 239657, 239658, 239659, 239660, 239661, 239662, 239663) c7(eq, dD, j); #c1(APOA2) c2(NP_001634) c3(apolipoprotein A-Il preproprotein) c4(119492, 119493) c5(119494, 119495, 119496, 119497, 119498, 119499) c6( ) c7(bL, dD, ef, ea, iW, oe, jx, aw, Zv, dG, j, fu, aj, v, ee, nS, eq, J, GK, Zu, ec, jy, dL, rB, bU, dQ, cW, Zt, Px, bJ, cl, cO, Fi, hr, at, R, ei, hE, B); #c1(APOA4) c2(NP_000473) c3(apolipoprotein A-IV precursor) c4(9443) c5(9444, 9445, 9446, 9447, 9448, 9449) c6( ) c7(bL, Kd, T, dD, jp, ea, iW, oe, e, aw, aj, bS, j, ct, ah, v, eq, nS, J, fU, nH, l, u, ec, aT, dL, ji, K, dQ, H, ex, RE, uX, A, cH, cW, aW, fO, fe, aq, iV, Z, YZ, cM, bJ, kT, ac, c, cO, Fi, ag, at, wx, cS, dy, ei, Zw, cE, B); #c1(APOA5) c2(NP_001160070) c3(apolipoprotein A-V precursor) c4(65064, 65065, 65066) c5(65067, 65068, 65069, 65070, 65071, 65072, 65073, 65074, 65075, 65076, 65077, 65078, 65079, 65080, 65081, 65082, 65083) c6( ) c7(bL, T, jv, dD, eR, Zz, ea, e, jx, aw, cz, vG, aj, v, j, eq, J, ad, dZ, dY, ec, M, h, jy, K, de, ae, af, Zx, hm, RE, eu, cH, Zy, fc, dq, aq, Z, ek, bJ, ar, ac, dC, cO, Fi, cS, aS, cP, ei, gT, B); #c1(APOB) c2(NP_000375) c3(apolipoprotein 8-100 precursor) c4(8274, 8275) c5(8276, 8277, 8278, 8279, 8280, 8281, 8282, 8283, 8284, 8285, 8286, 8287) c6( ) c7(T, ef, fR, Zz, ea, e, al, ci, GK, Gt, dY, u, vh, ZX, M, ZQ, K, dQ, H, hH, ex, eN, Bp, hm, ZH, aW, fc, nY, da, cJ, ZJ, uC, ZB, ZT, sU, k, pw, lS, dD, gU, ZP, ZF, ZK, aw, ZA, Wb, aj, ko, ee, ZS, cc, Zu, ec, lt, h, jy, dL, hB, ZN, db, RE, hi, CN, ZC, fe, aq, cM, KT, ZL, W, dC, cO, Fi, at, ZR, ZG, B, L, bL, vQ, ZV, eR, oe, jx, aA, cz, v, j, bX, so, nS, J, jF, ga, uT, aT, vH, cy, bK, DG, bq, sR, ZM, af, eu, ND, r, lo, wA, nC, fi, cl, ac, c, ZO, hr, tz, mf, cS, dy, Zl, gT, nZ, sY, qb, iW, HM, jN, vr, vG, ah, eq, gb, kL, hX, l, F, oz, ts, io, ZU, jo, vl, ux, ZE, cH, cW, cr, si, hp, Z, Hv, dr, qa, ek, gk, Ew, ZD, gZ, aS, cP, ei, y, ZW); #c1(APOBEC1) c2(NP_001291495) c3(C->U-editing enzyme APOBEC-1 isoform a) c4(116288) c5(116289, 116290, 116291, 116292, 116293, 116294) c6( ) c7(dD, ea, jx, bB, v, eq, ci, q, l, u, aT, h, dQ, Qe, B, s, cH, aW, r, aq, aF, ek, ck, Ew, bE, wD, gX, X); #c1(APOBEC2) c2(NP_006780) c3(probable C->U-editing enzyme APOBEC-2) c4(167999, 168000) c5(168001, 168002, 168003, 168004, 168005, 168006, 168007, 168008, 168009, 168010, 168011, 168012) c6( ) c7(ck, av, BC, BD, oV, e, fd, aq, hF, B); #c1(APOBEC3A_B) c2(XP_011545347) c3(probable DNA dC->du-editing enzyme APOBEC-3A isoform X2) c4(344117) c5(344118, 344119, 344120, 344121, 344122, 344123, 344124, 344125, 344126, 344127, 344128, 344129, 344130, 344131) c6( ) c7(T, aW, yk, aT, ZQ, gl, h, t); #c1(APOBEC3B) c2(NP_001257340) c3(DNA dC->du-editing enzyme APOBEC-38 isoform b) c4(95230) c5(95231, 95232, 95233, 95234, 95235, 95236) c6( ) c7(T, if, jh, aA, hy, jE, hX, iu, aE, dV, aT, h, dL, ey, aW, Y, ff, aq, af, en, iR, f, t, ck, eM, gn, m, eg, bt, B, hS); #c1(APOBEC3B) c2(NP_004891) c3(DNA dC->du-editing enzyme APOBEC-3B isoform a) c4(150601) c5(150602, 150603, 150604, 150605, 150606, 150607) c6( ) c7(T, if, jh, aA, hy, jE, hX, iu, aE, dV, aT, h, dl, ey, aW, Y, ff, aq, af, en, iR, f, t, ck, eM, gn, m, eg, bt, B, hS); #c1(APOBEC3C) c2(NP_055323) c3(DNA dC->dU-editing enzyme APOBEC-3C) c4(180483) c5(180484, 180485, 180486, 180487, 180488, 180489) c6( ) c7(ck, gl, fR, h); #c1(APOBEC3F) c2(NP_001006667) c3(DNA dC->du-editing enzyme APOBEC-3F isoform b) c4(22345) c5(22346, 22347, 22348, 22349, 22350, 22351) c6( ) c7(ck, Kd, fR, cz, h, hX); #c1(APOBEC3F) c2(NP_660341) c3(DNA dC->du-editing enzyme APOBEC-3F isoform a) c4(241014) c5(241015, 241016, 241017, 241018, 241019, 241020) c6( ) c7(ck, Kd, FR, cz, h, hX); #c1(APOBEC3G) c2(NP_068594) c3(DNA dC->du-editing enzyme APOBEC-3G) c4(209750) c5(209751, 209752, 209753, 209754, 209755, 209756, 209757, 209758, 209759, 209760) c6( ) c7(ck, Kd, iQ, fR, hX, gN, r, h, aq, lo, jh, gn, jE, q, iR, bt, B, vi); #c1(APOBEC3H) c2(NP_001159474) c3(DNA dC->du-editing enzyme APOBEC-3H isoform SV-182) c4(64148) c5(64149, 64150, 64151, 64152, 64153, 64154) c6( ) c7(h, fR); #c1(APOBEC3H) c2(NP_001159476) c3(DNA dC->dU-editing enzyme APOBEC-3H isoform SV-154) c4(64155) c5(64156, 64157, 64158, 64159, 64160, 64161) c6( ) c7(h, fR); #c1(APOBEC3H) c2(NP_861438) c3(DNA dC->du-editing enzyme APOBEC-3H isoform SV-183) c4(255565) c5(255566, 255567, 255568, 255569, 255570, 255571) c6( ) c7(h, fR); #c1(APOBEC3H) c2(XP_011528294) c3(DNA dC->du-editing enzyme APOBEC-3H isoform X1) c4(323366) c5(323367, 323368, 323369, 323370, 323371, 323372) c6( ) c7(h, fR); #c1(APOBR) c2(NP_061160) c3(apolipoprotein B receptor) c4(199653, 199654, 199655) c5(199656, 199657, 199658, 199659, 199660, 199661, 199662, 199663, 199664, 199665, 199666, 199667, 199668, 199669, 199670, 199671, 199672, 199673) c6( ) c7(bM, dD, cH, ea, v, eq); #c1(APOC1) c2(NP_001636) c3(apolipoprotein C-1 precursor) c4(119500, 119501) c5(119502, 119503, 119504, 119505, 119506, 119507, 119508, 119509, 119510, 119511, 119512, 119513) c6( ) c7(bL, aC, dD, fR, Zz, e, jx, cz, Zv, v, j, eq, C, fr, nS, V, fK, l, aE, u, jy, iY, ey, dQ, af, li, A, qt, fc, kp, eE, dR, es, bb, tm, cP, ei, ZY, ZZ); #c1(APOC2) c2(NP_000474) c3(apolipoprotein C-Il precursor) c4(9450) c5(9451, 9452, 9453, 9454, 9455, 9456) c6( ) c7(bL, T, dD, ZV, rG, Zz, jp, ea, aaa, e, jx, vG, j, eq, nS, V, eA, iu, mj, ec, sO, K, dQ, aab, af, Zx, aae, aad, aq, g, t, aac, dr, dR, bF, ar, cl, mf, m, ei, ZG, B); #c1(APOC3) c2(NP_000031) c3 (apolipoprotein C-Ill precursor) c4(282, 283, 284) c5(285, 286, 287, 288, 289, 290, 291, 292, 293, 294, 295, 296, 297, 298, 299, 300, 301, 302, 303, 304, 305, 306) c6(307, 308) c7(bL, sY, T, dD, eR, Zz, aag, ea, oe, e, jx, aw, cz, vG, Zv, dG, j, aj, v, eq, J, GK, td, hX, nH, l, F, Zu, Mm, u, ec, TB, av, aah, dv, aT, h, cy, dQ, af, A, lR, aai, cH, aW, fc, hl, r, aaf, aq, cJ, cM, uC, GR, bJ, iO, kT, GG, W, c, ac, cO, Fi, hr, gZ, cS, aS, cP, dy, bC, gT, ei, B); #c1(APOC4) c2(NP_001637) c3 (apolipoprotein C-IV precursor) c4(119514) c5(119515, 119516, 119517, 119518, 119519, 119520, 119521, 119522, 119523, 119524, 119525, 119526, 119527, 119528, 119529, 119530, 119531, 119532) c6( ) c7(bL, e, jx, fc, aq, j, cP, ei, nS, B); #c1(APOD) c2(NP_001638) c3(apolipoprotein D precursor) c4(119533, 119534) c5(119535, 119536, 119537, 119538, 119539, 119540, 119541, 119542, 119543, 119544, 119545, 119546) c6( ) c7(bL, T, rG, qy, v, j, ci, V, cd, xt, q, fK, aR, st, ad, Hx, eA, aT, PD, jy, aaj, K, an, he, ae, af, Uf, RT, s, Vx, aW, eH, fc, r, eF, cm, Hp, ac, PI, U, cP, hh, cB, B); #c1(APOE) c2(NP_001289617) c3(apolipoprotein E isoform a precursor) c4(114316) c5(114317, 114318, 114319, 114320, 114321, 114322, (14323, 114324, 114325, 114326, 114327) c6( ) c7(T, ef, abr, ea, abM, bO, Vd, aY, aao, fm, dV, hH, nY, abn, vs, GR, jV, wE, cj, lS, act, eg, nD, dt, aaJ, cQ, aj, cd, sW, KN, Ak, acb, aaQ, ey, dd, aak, sm, fe, hq, iO, abl, dB, Fi, Ei, Be, abR, WI, abW, jv, abc, cz, aA, bX, abC, q, PW, aas, kf, yu, cy, aaw, eu, bA, bf, abs, abo, bg, iy, lo, aby, pb, sk, rR, iQ, ac, mG, tz, aan, aaX, wi, acv, aag, iW, jN, jh, Hn, dG, uu, fu, xt, ad, hX, abZ, HF, Dq, Ot, abq, ZU, Aa, HD, cH, cW, aaQ, ff, gC, dq, EZ, iV, acx, bz, abA, ag, dX, lC, iu, oO, bM, aaM, Zz, xo, e, bB, ur, abD, sD, vh, Gb, bc, KC, up, dH, fc, hl, as, Eg, cm, eY, bb, Kv, xD, abS, mx, vy, wx, hh, abh, dD, n, QL, nA, ee, Za, jw, acf, Mo, b, no, Hx, ec, acm, RN, Xe, acA, eV, abK, acd, abf, A, Ib, ax, yq, vo, aap, abV, dC, cA, cO, fa, aar, at, eo, tm, sa, B, ek, tq, vQ, aba, wL, ex, GC, ti, v, aco, V, di, DK, WY, wN, rS, wP, ae, lr, hM, HL, uh, acB, aaQ, t, sk, acp, el, ck, cl, xb, vi, HQ, nZ, aat, acI, vG, OR, eq, abe, aaq, abP, sj, Jc, abz, d, abB, kD, xH, Z, Hv, dr, gk, yE, aam, aaG, gZ, MJ, y, aax, CW, sH, abT, abY, aC, fR, aaH, al, l, gf, aby, Ul, abH, u, lu, acc, wM, M, K, GO, wt, mH, aci, aaC, Zc, abx, g, aaD, uC, Uk, aaS, acw, aau, ZB, abw, Gs, U, m, abX, FW, ZF, Ol, aw, NT, fN, Zv, abd, JW, fK, nV, xc, lt, acg, aaA, vd, wb, ZN, RE, tx, ack, cM, ar, kT, W, bo, abt, gv, abd, ZG, L, ach, fM, ZV, oe, jx, qQ, Nt, abN, wo, j, acn, il, J, jF, Mg, MR, lg, hb, nX, jZ, bq, abu, kl, Y, r, en, wA, bN, Qi, tj, sn, c, hc, dh, Da, cS, dy, gT, sY, aaT, Pt, aaz, aaQ, bS, ah, iG, vN, dP, fU, acu, oj, xp, abm, cx, abU, vl, uX, lR, cr, FX, qa, dR, nb, sG, ei, xg, tQ, acz, abJ, eb, aR, acs, dY, aU, abj, aaW, qM, dQ, H, eN, Bp, hm, EN, acr, zQ, aW, ace, aaN, da, PG, abE, aaQ, abL, abl, nw, mk, pw, abp, abG, abO, R, gD, cZ, QR, wh, My, xM, Wb, mN, Mi, ct, fr, acy, eW, abb, ke, Gm, Zu, kl, h, jy, dL, hB, he, db, Df, Um, wC, Fn, ZC, aq, aay, dw, bJ, aaR, vD, bm, el, bD, bL, G, eR, dk, sM, cR, hy, OY, C, so, aaV, ga, aaf, acj, uT, aaY, HC, aT, Wk, HG, acq, bK, an, af, ak, s, abi, la, aaE, f, dS, pH, wg, pZ, fP, mf, Kd, qb, abF, ha, vr, dZ, kL, av, abk, l, F, ot, aaU, aaZ, oz, sO, NS, nH, ts, abg, vw, aak, aca, aaB, aav, aaP, eZ, yy, gl, aF, eK, es, bE, pE, dJ, nT, aS, tr, pk, Zw, X, us); #c1(APOE) c2(NP_001289618) c3(apolipoprotein E isoform b precursor) c4(114328, 114329, 114330) c5(114331, 114332, 114333, 114334, 114335, 114336, 114337, 114338, 114339, 114340, 114341, 114342, 114343, 114344, 114345, 114346, 114347, 114348, 114349, 114350, 114351) c6( ) c7(T, ef, abr, ea, abM, bO, Vd, aY, aao, fm, dV, hH, nY, abn, vs, GR, jV, wE, cj, lS, act, eg, nD, dt, aaQ, cQ, aj, cd, sW, KN, Ak, acb, aaQ, ey, dd, aak, sm, fe, hq, iO, abl, dB, Fi, Ei, Be, abR, WI, abW, jv, abc, cz, aA, bX, abC, q, PW, aas, kf, yu, cy, aaw, eu, bA, bf, abs, abo, bg, iy, lo, aby, pb, sk, rR, iQ, ac, mG, tz, aan, aaX, wi, acv, aag, iW, jN, jh, Hn, dG, uu, fu, xt, ad, hX, abZ, HF, Dq, Ot, abq, ZU, Aa, HD, cH, cW, aaQ, ff, gC, dq, EZ, iV, acx, bz, abA, ag, dX, lC, iu, oO, bM, aaM, Zz, xo, e, bB, ur, abD, sD, vh, Gb, bc, KC, up, dH, fc, hl, as, Eg, cm, eY, bb, Kv, xD, abS, mx, vy, wx, hh, abh, dD, n, QL, nA, ee, Za, jw, acf, Mo, b, no, Hx, ec, acm, RN, Xe, acA, eV, abK, acd, abf, A, lb, ax, yq, vo, aap, abV, dC, cA, cO, fa, aar, at, eo, tm, sa, B, ek, tq, vQ, aba, wL, eX, GC, ti, v, aco, V, di, DK, WY, wN, rS, wP, ae, lr, hM, HL, uh, acB, aaD, t, sk, acp, el, ck, cl, xb, vi, HQ, nZ, aat, acI, vG, oR, eq, abe, aaq, abP, sj, Jc, abz, d, abB, kD, xH, Z, Hv, dr, gk, yE, aam, aaG, gZ, MJ, y, aax, CW, sH, abT, abY, aC, fR, aaH, al, l, gf, abv, Ul, abH, u, lu, acc, wM, M, K, GO, wt, mH, aci, aaC, Zc, abx, g, aaD, uC, Uk, aaS, acw, aau, ZB, abw, Gs, U, m, abX, FW, ZF, DI, aw, NT, fN, Zv, abQ, JW, fK, nV, xc, lt, acg, aaA, vd, wb, ZN, RE, tx, ack, cM, ar, kT, W, bo, abt, gv, abd, ZG, L, ach, fM, ZV, oe, jx, qQ, Nt, abN, wo, j, acn, il, J, jF, Mg, MR, lg, hb, nX, jZ, bq, abu, kl, Y, r, en, wA, bN, Qi, tj, sn, c, hc, dh, Da, cS, dy, gT, sY, aaT, Pt, aaz, aaQ, bS, ah, iG, vN, dP, fU, acu, oj, xp, abm, cx, abU, vl, uX, lR, cr, FX, qa, dR, nb, sG, ei, xg, tQ, acz, abJ, eb, aR, acs, dY, aU, abj, aaW, qM, d, H, eN, Bp, hm, EN, acr, zQ, aW, ace, aaN, da, PG, abE, aaQ, abL, abl, nw, mk, pw, abp, abG, abO, R, gD, cZ, QR, wh, My, xM, Wb, mN, Mi, ct, fr, acy, eW, abb, ke, Gm, Zu, kl, h, jy, dL, hB, he, db, Df, Um, WC, Fn, ZC, aq, aay, dw, bJ, aaR, vD, bm, el, bD, bL, rG, eR, dk, sM, cR, hy, OY, C, so, aaV, ga, aaF, acj, uT, aaY, HC, aT, Wk, HG, acq, bK, an, af, ak, s, abi, la, aaE, f, dS, pH, wg, pZ, fP, mf, Kd, qb, abF, ha, vr, dZ, KL, av, abk, l, F, ot, aaU, aaZ, oz, sO, NS, nH, ts, abg, vw, aak, aca, aaB, aav, aaP, eZ, yy, gl, aF, ek, es, bE, pE, dJ, nT, aS, tr, pK, Zw, X, us); #c1(APOF) c2(NP_001629) c3(apolipoprotein F precursor) c4(119360, 119361) c5(119362, 119363, 119364, 119365, 119366, 119367, 119368, 119369, 119370, 119371, 119372, 119373, 119374, 119375, 119376, 119377) c6(119378, 119379, 119380, 119381) c7(W, cH, j, dQ, af, vi); #c1(APOH) c2(NP_000033) c3(beta-2-glycoprotein 1 precursor) c4(309) c5(310, 311, 312, 313, 314, 315) c6( ) c7(dD, eR, fR, ea, iW, Hc, e, DV, al, cz, cR, fN, aj, vr, eq, ez, mC, J, aw, ec, K, jj, hH, OV, Y, sq, eZ, kD, aq, YG, iV, kk, acD, ek, acC, ac, cj, dX, aS, fl, ei, B); #c1(APOL1) c2(NP_001130013) c3(apolipoprotein L1 isoform c) c4(52433) c5(52434, 52435, 52436, 52437, 52438, 52439, 52440, 52441, 52442) c6( ) c7(bL, T, dD, eR, qb, ea, e, cz, vG, cQ, dG, j, ah, aj, eq, fr, aY, ye, J, fU, acE, vw, h, ey, XZ, CG, acF, eZ, lo, qa, sk, cO, MM, dy, DQ); #c1(APOL1) c2(NP_003652) c3(apolipoprotein L1 isoform a precursor) c4(139940, 139941) c5(139942, 139943, 139944, 139945, 139946, 139947, 139948, 139949, 139950, 139951, 139952, 139953, 139954, 139955, 139956, 139957, 139958, 139959, 139960, 139961, 139962, 139963, 139964, 139965, 139966, 139967, 139968, 139969, 139970, 139971, 139972, 139973, 139974, 139975, 139976, 139977, 139978, 139979, 139980, 139981, 139982, 139983) c6( ) c7(bL, T, dD, eR, qb, ea, e, cz, vG, cQ, dG, j, ah, aj, eq, fr, aY, ye, J, fU, acE, vw, h, ey, XZ, CG, acF, eZ, lo, qa, sk, co, MM, dy, DQ); #c1(APOL1) c2(NP_663318) c3(apolipoprotein Li isoform b precursor) c4(241198, 24/199, 241200) c5(241201, 241202, 241203, 241204, 241205, 241206, 241207, 241208, 241209, 24/210, 241211, 241212, 241213, 241214, 241215, 241216, 241217, 241218, 241219, 241220, 24/221, 241222, 241223) c6( ) c7(bL, T, dD, eR, qb, ea, e, cz, vG, cQ, dG, j, ah, aj, eq, fr, aY, ye, J, fU, acE, vw, h, ey, XZ, CG, acF, eZ, lo, qa, sk, co, MM, dy, DQ); #c1(APOL1) c2(XP_011528780) c3(apolipoprotein L1 isoform X2) c4(324084) c5(324085, 324086, 324087, 324088, 324089, 324090, 324091) c6( ) c7(bL, T, dD, eR, qb, ea, e, cz, vG, c, dG, j, ah, aj, eq, fr, aY, ye, J, fU, acE, vw, h, ey, XZ, CG, acF, eZ, lo, qa, sk, cO, MM, dy, DQ); #c1(APOL2) c2(NP_663612) c3(apolipoprotein L2) c4(241224, 241225, 24/226, 241227) c5(241228, 241229, 241230, 241231, 241232, 241233, 241234, 241235, 241236, 241237, 241238, 241239, 241240, 241241, 241242, 241243, 241244, 241245, 241246, 241247, 241248, 241249, 241250, 241251, 241252, 241253, 241254, 241255, 241256, 241257, 241258, 241259, 241260, 241261, 241262, 241263) c6(24/264) c7(jb, cu, acH, acI, acG); #c1(APOL3) c2(NP_663615) c3(apolipoprotein L3 isoform 1) c4(241317) c5(241318, 241319, 241320, 241321, 241322, 241323, 241324, 241325, 241326, 241327) c6( ) c7(T, ae, ad); #c1(APOL3) c2(NP_663617) c3(apolipoprotein L3 isoform 3) c4( ) c5(241328, 241329, 241330, 241331, 241332, 241333, 241334, 241335, 241336, 241337, 241338) c6( ) c7(T, ae, ad); #c1(APOL4) c2(NP_085146) c3(apolipoprotein L4 isoform 1) c4(219417, 219418, 219419, 219420, 219421, 219422) c5(219423, 219424, 219425, 219426, 219427, 219428, 219429, 219430, 219431, 219432, 219433, 219434, 219435, 219436, 219437, 219438, 219439, 219440, 219441, 219442, 219443, 219444, 219445, 219446, 219447, 219448, 219449, 219450, 219451, 219452, 219453, 219454, 219455, 219456, 219457, 219458, 219459, 219460, 219461, 219462, 219463, 219464, 219465, 219466, 219467, 219468, 219469, 219470, 219471, 219472, 219473, 219474, 219475, 219476) c6(219477, 219478, 219479, 219480) c7(oe, eR); #c1(APOL4) c2(NP_663693) c3(apolipoprotein L4 isoform 2 precursor) c4(241403, 241404) c5(241405, 241406, 241407, 241408, 241409, 241410, 241411, 241412, 241413, 241414, 241415, 241416, 241417, 241418, 241419, 241420) c6( ) c7(oe, eR); #c1(APOL6) c2(NP_085144) c3 (apolipoprotein L6) c4(219409) c5(219410, 219411, 219412, 219413, 219414, 219415, 219416) c6( ) c7(ds, e); #c1(APOL01) c2(NP_001123887) c3(apolipoprotein L domain-containing protein 1 isoform 1) c4(47969) c5(47970, 47971, 47972, 47973, 47974, 47975) c6( ) c7(g); #c1(APOLD1) c2(NP_110444) c3(apolipoprotein L domain-containing protein 1 isoform 2) c4(220390) c5(220391, 220392, 220393, 220394, 220395, 220396) c6( ) c7(g); #c1(APOM) c2(NP_001243098) c3(apolipoprotein M isoform 2) c4( ) c5(89869, 89870, 89871, 89872, 89873, 89874, 89875, 89876, 89877, 89878, 89879, 89880, 89881, 89882, 89883, 89884, 89885, 89886, 89887, 89888, 89889, 89890, 89891, 89892, 89893, 89894, 89895, 89896, 89897, 89898, 89899, 89900, 89901, 89902, 89903, 89904, 89905, 89906, 89907, 89908, 89909, 89910, 89911, 89912, 89913, 89914, 89915, 89916, 89917, 89918, 89919, 89920, 89921, 89922, 89923, 89924, 89925, 89926, 89927, 89928, 89929, 89930, 89931, 89932, 89933, 89934, 89935, 89936, 89937, 89938, 89939, 89940, 89941) c6(89942, 89943, 89944, 89945, 89946, 89947, 89948, 89949, 89950, 89951, 89952, 89953, 89954, 89955, 89956, 89957, 89958, 89959, 89960, 89961, 89962, 89963, 89964, 89965, 89966, 89967, 89968) c7(bL, iu, dD, A, fR, af, ea, e, M, lG, aj, dG, j, eq, y, B, J, vi); #c1(APOM) c2(NP_061974) c3(apolipoprotein M isoform 1) c4(201363, 201364, 201365) c5(201366, 201367, 201368, 201369, 201370, 201371, 201372, 201373, 201374, 201375, 201376, 201377, 201378, 201379, 201380, 201381, 201382, 201383, 201384, 201385, 201386, 201387, 201388, 201389, 201390, 201391, 201392, 201393, 201394, 201395, 201396, 201397, 201398, 201399, 201400, 201401, 201402, 201403, 201404, 201405, 201406, 201407, 201408) c6(201409, 201410, 201411, 201412, 201413, 201414, 201415, 201416, 201417, 201418, 201419, 201420, 201421, 201422) c7(bL, iu, dD, A, fR, af, ea, e, M, lG, aj, dG, j, eq, y, B, J, vi); #c1(APOD) c2(NP_077027) c3 (apolipoprotein 0 precursor) c4(214813, 214814) c5(214815, 214816, 214817, 214818, 214819, 214820, 214821, 214822, 214823, 214824, 214825, 214826, 214827, 214828, 214829, 214830, 214831, 214832) c6( ) c7(v, af, cc, EQ); #c1(APO0) c2(XP_011543888) c3(apolipoprotein 0 isoform X1) c4(343062) c5( ) c6( ) c7(v, af, cc, EQ); #c1(APOPT1) c2(NP_115750) c3(apoptogenic protein 1, mitochondrial isoform 1) c4(223802, 223803) c5(223804, 223805, 223806, 223807, 223808, 223809, 223810, 223811, 223812, 223813, 223814, 223815) c6( ) c7(kC, acJ); #c1(APP) c2(NP_000475) c3(amyloid beta A4 protein isoform a precursor) c4(9457) c5(9458, 9459, 9460, 9461, 9462, 9463, 9464, 9465, 9466, 9467, 9468, 9469) c6( ) c7(T, fR, aaH, ea, e, l, ci, bh, aY, aR, aao, dE, Fo, aU, K, GO, H, bc, mH, acN, aW, acO, bp, Ku, cm, ba, bb, acS, U, aL, R, bi, hh, dt, gU, dD, acT, aO, Ol, aj, bd, PJ, fK, eW, aN, h, OV, A, acR, aJ, aq, kk, acM, hq, W, fa, bm, al, B, ek, L, acI, aA, cz, v, j, C, J, di, MR, hb, WY, HC, aT, vH, aaw, bK, UN, an, aH, lr, af, ak, bf, vT, Y, aM, bg, acP, wA, f, bN, ck, ac, c, hr, mf, gT, bt, hl, aZ, aaQ, acu, eq, xt, ack, ot, aV, sO, Dq, oj, bj, aQ, ach, ji, d, BO, aP, cr, gl, aF, KM, wl, dJ, X, aS, be, ei, Gp, y, xg, CW); #c1(APP) c2(NP_001129488) c3(amyloid beta A4 protein isoform d) c4(51595) c5(51596, 51597, 51598, 51599, 51600, 51601, 51602, 51603, 51604, 51605, 51606, 51607) c6( ) c7(T, fR, aaH, ea, e, I, ci, bh, aY, aR, aao, dE, Fo, aU, K, GO, H, bc, mH, acN, aW, acO, bp, Ku, cm, ba, bb, acS, U, aL, R, bi, hh, dt, gU, dD, acT, aO, Ol, aj, bd, PJ, fK, eW, aN, h, OV, A, acR, aJ, aq, kk, acM, hq, W, fa, bm, al, B, ek, L, acI, aA, cz, v, j, C, J, di, MR, hb, WY, HC, aT, vH, aaw, bK, UN, an, aH, lr, af, ak, bf, vT, Y, aM, bg, acP, wA, f, bN, ck, ac, c, hr, mf, gT, bt, hl, aZ, aaQ, acU, eq, xt, ack, ot, aV, sO, Dq, oj, bj, aQ, ach, ji, d, BO, aP, cr, gl, aF, KM, wl, dJ, X, aS, be, ei, Gp, y, xg, CW); #c1(APP) c2(NP_001129601) c3(amyloid beta A4 protein isoform e precursor) c4(51984) c5(51985, 51986, 51987, 51988, 51989, 51990, 51991, 51992, 51993, 51994, 51995, 51996, 51997, 51998, 51999, 52000, 52001, 52002) c6( ) c7(T, FR, aaH, ea, e, l, ci, bh, aY, aR, aao, dE, Fo, aU, K, GO, H, bc, mH, acN, aW, acO, bp, Ku, cm, ba, bb, acS, U, aL, R, bi, hh, dt, gU, dD, acT, aO, Ol, aj, bd, PJ, fK, eW, aN, h, OV, A, acR, aJ, aq, kk, acM, hq, W, fa, bm, al, B, ek, L, acI, aA, cz, v, j, C, J, di, MR, hb, WY, HC, aT, vH, aaw, bK, UN, an, aH, lr, af, ak, bf, vT, Y, aM, bg, acP, wA, f, bN, ck, ac, c, hr, mf, gT, bt, hl, aZ, aaQ, acu, eq, xt, ack, ot, aV, sO, Dq, oj, bj, aQ, acQ, ji, d, BO, aP, cr, gl, aF, KM, wQ, dJ, X, aS, be, ei, Gp, y, xg, CW); #c1(APP) c2(NP_001129602) c3(amyloid beta A4 protein isoform f precursor) c4(52003, 52004) c5(52005, 52006, 52007, 52008, 52009, 52010) c6( ) c7(T, fR, aaH, ea, e, l, ci, bh, aY, aR, aao, dE, Fo, aU, K, GO, H, bc, mH, acN, aW, acO, bp, Ku, cm, ba, bb, acS, U, aL, R, bi, hh, dt, gU, dD, acT, aO, Ol, aj, bd, PJ, fK, eW, aN, h, OV, A, acR, aJ, aq, kk, acM, hq, W, fa, bm, al, B, ek, L, acI, aA, cz, v, j, C, J, di, MR, hb, WY, HC, aT, vH, aaw, bK, UN, an, aH, lr, af, ak, bf, vT, Y, aM, bg, acP, wA, f, bN, ck, ac, c, hr, mf, gT, bt, hl, aZ, aaQ, acu, eq, xt, ack, ot, aV, sO, Dq, oj, bj, aQ, acd, ji, d, BO, aP, cr, gl, aF, KM, wl, dJ, X, aS, be, ei, Gp, y, xg, CW); #c1(APP) c2(NP_001129603) c3(amyloid beta A4 protein isoform g) c4(52011) c5( ) c6( ) c7(T, fR, aaH, ea, e, l, ci, bh, aY, aR, aao, dE, Fo, aU, K, GO, H, bc, mH, acN, aW, acO, bp, Ku, cm, ba, bb, acS, U, aL, R, bi, hh, dt, gU, dD, acT, aO, Ol, aj, bd, PJ, fK, eW, aN, h, OV, A, acR, aJ, aq, kk, acM, hq, W, fa, bm, al, B, ek, L, acI, aA, cz, v, j, C, J, di, MR, hb, WY, HC, aT, vH, aaw, bK, UN, an, aH, lr, af, ak, bf, vT, Y, aM, bg, acP, wA, f, bN, ck, ac, c, hr, mf, gT, bt, hl, aZ, aaQ, acU, eq, xt, ack, ot, aV, sO, Dq, oj, bj, aQ, acQ, ji, d, BO, aP, cr, gl, aF, KM, wl, du, X, aS, be, ei, Gp, y, xg, CW); #c1(APP) c2(NP_001191230) c3(amyloid beta A4 protein isoform h precursor) c4(81170) c5(81171, 81172, 81173, 81174, 81175, 81176) c6( ) c7(T, fR, aaH, ea, e, l, ci, bh, aY, aR, aao, dE, Fo, aU, K, GO, H, bc, mH, acN, aW, acO, bp, Ku, cm, ba, bb, acS, U, aL, R, bi, hh, dt, gU, dD, acT, aO, Ol, aj, bd, PJ, fK, eW, aN, h, OV, A, acR, aJ, aq, kk, acM, hq, W, fa, bm, al, B, ek, L, acI, aA, cz, v, j, C, J, di, MR, hb, WY, HC, aT, vH, aaw, bK, UN, an, aH, lr, af, ak, bf, vT, Y, aM, bg, acP, wA, f, bN, ck, ac, c, hr, mf, gT, bt, hl, aZ, aaQ, acU, eq, xt, ack, ot, aV, sO, Dq, oj, bj, aQ, acQ, ji, d, BO, aP, cr, gl, aF, KM, wQ, dJ, X, aS, be, ei, Gp, y, xg, CW); #c1(APP) c2(NP_958816) c3(amyloid beta A4 protein isoform b precursor) c4(262218) c5( ) c6( ) c7(T, fR, aaH, ea, e, l, ci, bh, aY, aR, aao, dE, Fo, aU, K, GO, H, bc, mH, acN, aW, acO, bp, Ku, cm, ba, bb, acS, U, aL, R, bi, hh, dt, gU, dD, acT, aO, Ol, aj, bd, PJ, fK, eW, aN, h, OV, A, acR, aJ, aq, kk, acM, hq, W, fa, bm, al, B, ek, L, acL, aA, cz, v, j, C, J, di, MR, hb, WY, HC, aT, vH, aaw, bK, UN, an, aH, lr, af, ak, bf, vT, Y, aM, bg, acP, wA, f, bN, ck, ac, c, hr, mf, gT, bt, hl, aZ, aal, acU, eq, xt, ack, ot, aV, sO, Dq, oj, bj, aQ, acI, ji, d, BO, aP, cr, gl, aF, KM, wl, dJ, X, aS, be, ei, Gp, y, xg, CW); #c1(APP) c2(NP_958817) c3(amyloid beta A4 protein isoform c precursor) c4(262219) c5( ) c6( ) c7(T, fR, aaH, ea, e, l, ci, bh, aY, aR, aao, dE, Fo, aU, K, GO, H, bc, mH, acN, aW, acO, bp, Ku, cm, ba, bb, acS, U, aL, R, bi, hh, dt, gU, dD, acT, aO, Ol, aj, bd, PJ, fK, eW, aN, h, OV, A, acR, aJ, aq, kk, acM, hq, W, fa, bm, al, B, ek, L, acI, aA, cz, v, j, C, J, di, MR, hb, WY, HC, aT, vH, aaw, bK, UN, an, aH, lr, af, ak, bf, vT, Y, aM, bg, acP, wA, f, bN, ck, ac, c, hr, mf, gT, bt, hl, aZ, aaQ, acu, eq, xt, ack, ot, aV, sO, Dq, oj, bj, aQ, ach, ji, d, BO, aP, cr, gl, aF, KM, wl, dJ, X, aS, be, ei, Gp, y, xg, CW); #c1(APPBP2) c2(NP_006371) c3(amyloid protein-binding protein 2 isoform 1) c4(164447) c5(164448, 164449, 164450, 164451, 164452, 164453, 164454, 164455) c6( ) c7(aW, cz, aT, lu, cd, ad); #c1(APPL1) c2(NP_036228) c3(DCC-interacting protein 13-alpha) c4(172460) c5(172461, 172462, 172463, 172464, 172465, 172466) c6( ) c7(bL, dD, ea, fc, cR, Mz, aj, dG, v, m, cP, eq, t, J); #c1(APPL2) c2(NP_001238833) c3(DCC-interacting protein (3-beta isoform 2) c4(87942) c5(87943, 87944, 87945, 87946, 87947, 87948) c6( ) c7(d, fc, ey, qF, cP, lB, eA, ad); #c1(APPL2) c2(NP_060641) c3(DCC-interacting protein 13-beta isoform 1) c4(196952) c5(196953, 196954, 196955, 196956, 196957, 196958) c6( ) c7(d, fc, ey, qF, cP, lB, eA, ad); #c1(APRT) c2(NP_000476) c3(adenine phosphoribosyltransferase isoform a) c4(9470, 9471) c5(9472, 9473, 9474, 9475, 9476, 9477) c6( ) c7(bM, T, ea, if, bO, bB, fo, ci, aY, add, x, acX, aE, ws, dv, K, EL, zy, adn, aW, Bw, fc, Ku, g, eE, eF, cm, DL, GR, iP, eM, nB, iT, wB, cj, k, m, lS, dD, Ol, vX, adk, aj, fr, ct, qN, adb, cd, adf, cc, A, fe, aq, CQ, Ld, JR, hq, bP, acY, bo, hR, HB, acZ, Po, B, it, tq, bL, fM, mr, jx, aA, cR, adi, v, ada, j, q, J, eA, nv, adl, aT, adm, jj, bq, ae, af, acV, kl, s, nO, RO, oV, Y, r, lo, t, kC, bN, EJ, ck, acW, ac, c, iM, MB, HK, fP, fD, ao, Kd, hl, iW, jh, Hn, bS, dG, jE, ade, eq, Fb, adg, MC, ad, hX, fU, hd, ib, vc, ji, lQ, nJ, d, je, eH, cr, dq, aF, Z, adj, HI, bz, dr, ek, adc, Ew, kg, bE, cP, ei, pk, lC, adh, kS, iu); #c1(APRT) c2(NP_001025189) c3(adenine phosphoribosyltransferase isoform b) c4( ) c5(29485, 29486, 29487, 29488, 29489, 29490) c6( ) c7(bM, T, ea, if, bO, bB, fo, ci, aY, add, x, acX, aE, ws, dv, K, EL, zy, adn, aW, Bw, fc, Ku, g, eE, eF, cm, DL, GR, iP, eM, nB, iT, wB, cj, k, m, lS, dD, Ol, vX, adk, aj, fr, ct, qN, adb, cd, adf, cc, A, fe, aq, CQ, Ld, JR, hq, bP, acY, bo, hR, HB, acZ, Po, B, it, tq, bL, fM, mr, jx, aA, cR, adi, v, ada, j, q, J, eA, nv, adl, aT, adm, j, bq, ae, af, acV, kl, s, nO, RO, oV, Y, r, lo, t, kC, bN, EJ, ck, acW, ac, c, iM, MB, HK, fP, fD, ao, Kd, hl, iW, jh, Hn, bS, dG, jE, ade, eq, Fb, adg, MC, ad, hX, fU, hd, ib, vc, ji, lQ, nJ, d, je, eH, cr, dq, aF, Z, adj, HI, bz, dr, ek, adc, Ew, kg, bE, cP, ei, pk, lC, adh, kS, iu); #c1(APTX) c2(NP_001182177) c3(aprataxin isoform e) c4(74640, 74641, 74642) c5(74643, 74644, 74645, 74646, 74647, 74648, 74649, 74650, 74651, 74652, 74653, 74654, 74655, 74656, 74657, 74658, 74659) c6( ) c7(aC, ads, qy, GC, EF, n, fu, bX, ado, aR, ad, aU, Sq, DG, K, KU, bV, EG, adt, d, adr, cH, KH, adp, EH, kC, adq, wT, ky, kB, U, KA, bC, bt, KF); #c1(APTX) c2(NP_001182179) c3(aprataxin isoform f) c4(74660) c5(74661, 74662, 74663, 74664, 74665, 74666) c6( ) c7(aC, ads, qy, GC, EF, n, fu, bX, ado, aR, ad, aU, Sq, DG, K, KU, bV, EG, adt, d, adr, cH, KH, adp, EH, kC, adq, wT, ky, kB, U, KA, bC, bt, KF); #c1(APTX) c2(NP_001182180) c3(aprataxin isoform g) c4( ) c5(74667, 74668, 74669, 74670, 74671) c6( ) c7(aC, ads, qy, GC, EF, n, fu, bX, ado, aR, ad, aU, Sq, DG, K, KU, bV, EG, adt, d, adr, cH, KH, adp, EH, kC, adq, wT, ky, kB, U, KA, bC, bt, KF); #c1(APTX) c2(NP_001182181) c3 (aprataxin isoform i) c4(74672) c5(74673, 74674, 74675, 74676, 74677, 74678) c6( ) c7(aC, ads, qy, GC, EF, n, fu, bX, ado, aR, ad, aU, Sq, DG, K, KU, bV, EG, adt, d, adr, cH, KH, adp, EH, kC, adq, wT, ky, kB, U, KA, bC, bt, KF); #c1(APTX) c2(NP_778239) c3(aprataxin isoform b) c4(251505, 251506) c5(251507, 251508, 251509, 251510, 251511, 251512, 251513, 251514, 251515) c6( ) c7(aC, ads, qy, GC, EF, n, fu, bX, ado, aR, ad, aU, Sq, DG, K, KU, bV, EG, adt, d, adr, cH, KH, adp, EH, kC, adq, wT, ky, kB, U, KA, bC, bt, KF); #c1(APTX) c2(XP_006716854) c3(aprataxin isoform X1) c4( ) c5(290521, 290522, 290523, 290524, 290525, 290526, 290527, 290528, 290529, 290530, 290531) c6( ) c7(aC, ads, qy, GC, EF, n, fu, bX, ado, aR, ad, aU, Sq, DG, K, KU, bV, EG, adt, d, adr, cH, KH, adp, EH, kC, adq, wT, ky, kB, U, KA, bC, bt, KF); #c1(AQP1) c2(NP_001171989) c3(aquaporin-1 isoform 2) c4( ) c5(72150, 72151, 72152, 72153, 72154) c6( ) c7(cZ, T, dD, ul, ea, adv, ha, al, cz, fF, bB, aj, eq, ci, fr, zP, Rk, q, ad, qW, eA, ip, ady, aaF, ib, RI, jz, Qk, qK, aT, K, dd, bq, yj, ae, af, adx, ak, nP, zQ, d, nO, aW, Y, vj, xs, adw, cr, ff, kD, en, cl, g, t, as, bz, adz, adA, dS, adu, gk, pZ, zr, dB, Fh, bE, eM, uR, hr, cj, hL, dy, mk, y, cB); #c1(AQP1) c2(NP_001171990) c3(aquaporin-1 isoform 3) c4(72155, 72156) c5(72157, 72158, 72159, 72160, 72161, 72162, 72163) c6( ) c7(cZ, T, dD, ul, ea, adv, ha, al, cz, fF, bB, aj, eq, ci, fr, zP, Rk, q, ad, qW, eA, ip, ady, aaF, ib, RI, jz, Qk, qK, aT, K, dd, bq, yj, ae, af, adx, ak, nP, zQ, d, nO, aW, Y, vj, xs, adw, cr, ff, kD, en, cl, g, t, as, bz, adz, adA, dS, adu, gk, pZ, zr, dB, Fh, bE, eM, uR, hr, cj, hL, dy, mk, y, cB); #c1(AQP1) c2(NP_00171991) c3(aquaporin-1 isoform 4) c4(72164) c5(72165, 72166, 72167, 72168, 72169, 72170) c6( ) c7(cZ, T, dD, ul, ea, adv, ha, al, cz, fF, bB, aj, eq, ci, fr, zP, Rk, q, ad, qW, eA, ip, ady, aaF, ib, RI, jz, Qk, qK, aT, K, dd, bq, yj, ae, af, adx, ak, nP, zQ, d, nO, aW, Y, vj, xs, adw, cr, ff, kD, en, cl, g, t, as, bz, adz, adA, dS, adu, gk, pZ, zr, dB, Fh, bE, eM, uR, hr, cj, hL, dy, mk, y, cB); #c1(AQP1) c2(NP_932766) c3(aquaporin-1 isoform 1) c4(258997, 258998, 258999, 259000, 259001) c5(259002, 259003, 259004, 259005, 259006, 259007, 259008, 259009, 259010, 259011, 259012, 259013, 259014, 259015, 259016, 259017, 259018, 259019, 259020, 259021, 259022, 259023, 259024, 259025, 259026, 259027, 259028, 259029, 259030, 259031, 259032) c6(259033, 259034) c7(cZ, T, dD, ul, ea, adv, ha, al, cz, fF, bB, aj, eq, ci, fr, zP, Rk, q, ad, qW, eA, ip, ady, aaF, ib, RI, jz, Dk, qK, aT, K, dd, bq, yj, ae, af, adx, ak, nP, zQ, d, nO, aW, Y, vj, xs, adw, cr, ff, kD, en, cl, g, t, as, bz, adz, adA, dS, adu, gk, pZ, zr, dB, Fh, bE, eM, uR, hr, cj, hL, dy, mk, y, cB); #c1(AQP10) c2(NP_536354) c3(aquaporin-10) c4(231281, 231282) c5(231283, 231284, 231285, 231286, 231287, 231288, 231289, 231290, 231291, 231292, 231293, 231294) c6( ) c7(ac, aw, e, j, af, eu); #c1(AQP2) c2(NP_000477) c3(aquaporin-2) c4(9478) c5(9479, 9480, 9481, 9482, 9483, 9484) c6( ) c7(bL, sY, eR, Hm, al, cz, nA, cd, dP, cc, adC, adD, adB, bq, yj, adF, dq, adE, cl, Rp, uC, Fh, mG, dy, fy, adG); #c1(AQP3) c2(NP_004916) c3(aquaporin-3) c4(150755) c5(150756, 150757, 150758, 150759, 150760, 150761) c6( ) c7(T, sw, yk, Hc, ht, aA, dG, v, ci, qj, Lw, ad, co, ft, hd, adC, nv, qK, oW, aT, dV, dL, K, lQ, yj, ae, eu, Jl, gT, Y, aW, az, AQ, ff, aF, en, Sm, g, t, eE, iV, fy, eM, nB, Fh, hk, adH, uz, adg, nz, iu, pC); #c1(AQP4) c2(NP_001641) c3(aquaporin-4 isoform a) c4( ) c5(119574, 119575, 119576, 119577, 119578, 119579, 119580, 119581, 119582, 119583) c6( ) c7(ul, wi, adP, dA, AS, sC, ha, lr, cz, aj, uu, adK, mF, il, V, Rk, qW, aR, eA, pd, ip, ke, Mo, aaF, Vp, hd, adL, db, nv, adN, Ok, hD, lG, cy, abW, K, adJ, bq, H, adO, NM, Wq, bc, hm, adx, eu, ak, hM, nP, d, lR, xs, adM, uY, EZ, g, as, dw, f, bN, dS, adu, Hp, Hw, lt, ac, c, nB, dB, wl, Fh, eM, km, adl, vy, mk, hh, cB, gD); #c1(AQP5) c2(NP_001642) c3(aquaporin-5) c4(119584) c5(119585, 119586, 119587, 119588, 119589, 119590, 119591, 119592, 119593, 119594, 119595, 119596, 119597, 119598, 119599, 119600, 119601, 119602, 119603, 119604, 119605, 119606, 119607, 119608, 119609, 119610, 119611, 119612, 119613, 119614, 119615, 119616, 119617, 119618, 119619, 119620, 119621, 119622, 119623, 119624, 119625, 119626, 119627, 119628, 119629, 119630, 119631, 119632, 119633, 119634, 119635, 119636, 119637, 119638, 119639, 119640, 119641, 119642, 119643, 119644, 119645, 119646, 119647, 119648, 119649, 119650, 119651, 119652, 119653, 119654, 119655, 119656, 119657, 119658, 119659, 119660, 119661, 119662, 119663, 119664, 119665, 119666, 119667, 119668, 119669, 119670, 119671, 119672, 119673, 119674, 119675, 119676, 119677, 119678, 119679) c6(119680, 119681, 119682) c7(T, bB, j, C, q, ip, co, qK, aT, ab, add, K, eu, iU, iE, aW, ff, r, en, t, bz, eM, bE, wk, at, adR, mk, m, it); #c1(AQP6) c2(NP_001643) c3(aquaporin-6) c4(119683) c5(119684, 119685, 119686, (19687, 119688, 119689) c6( ) c7(rE, Qk, qc, adS); #c1(AQP7) c2(NP_001161) c3(aquaporin-7) c4(66316, 66317, 66318, 66319) c5( ) c6( ) c7(bL, d, dB, aZ, Y, Kk, cz, fl, nm, mf, dG, qM, v, eA, jj, adT); #c1(AQP7) c2(XP_011516167) c3(aquaporin-7 isoform X2) c4( ) c5(309413) c6( ) c7(bL, d, dB, aZ, Y, Kk, cz, fQ, nm, mf, dG, qM, v, eA, jj, adT); #c1(AQP7) c2(XP_011516169) c3(aquaporin-7 isoform X3) c4( ) c5(309414, 309415, 309416, 309417, 309418, 309419) c6( ) c7(bL, d, dB, aZ, Y, Kk, cz, fl, nm, mf, dG, qM, v, eA, jj, adT); #c1(AQP7) c2(XP_011516170) c3 (aquaporin-7 isoform X4) c4(309420, 309421) c5( ) c6( ) c7(bL, d, dB, aZ, Y, Kk, cz, fQ, nm, mf, dG, qM, v, eA, jj, adT); #c1(AQP8) c2(NP_001160) c3(aquaporin-8) c4( ) c5( ) c6( ) c7(kl, aZ, vj, qN, iV, eu); #c1(AQP9) c2(NP_066190) c3(aquaporin-9) c4(206831) c5(206832, 206833, 206834, 206835, 206836, 206837, 206838, 206839, 206840, 206841) c6( ) c7(bL, aZ, adU, dG, v, eA, nm, hn, jj, uV, adT, kl, d, iU, Y, vj, aq, dw, bz, ck, Hw, iT, dB, mf, Pk, bt, B); #c1(AR) c2(NP_000035) c3(androgen receptor isoform 1) c4(331, 332, 333, 334, 335, 336) c5(337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374) c6( ) c7(gh, T, aey, ea, bO, fd, aeu, ci, YD, xy, aex, aE, lv, hu, li, nY, uY, PQ, afg, nB, aeB, lq, cX, ht, aj, ml, aeX, aeI, adX, Ak, ey, ox, Dz, bl, on, yi, mR, Ln, pm, zZ, hq, dB, JA, aA, cz, AD, q, aew, aeh, kf, cy, jj, eu, bA, bf, aek, aeS, eS, Lc, cl, pt, rR, acW, ac, iQ, bt, aag, iW, KW, jh, adW, KO, uu, sg, ad, afb, hX, vJ, lQ, wm, aeT, Ub, cW, PE, ff, afi, aeV, bz, DF, aeg, aeH, aed, iu, ne, jr, Fv, if, bB, cL, bh, Ul, ip, ws, aeI, aeN, xQ, aeR, cm, qJ, Kv, AV, vA, mx, vy, in, ik, dD, gU, wu, n, oY, nA, no, Do, aes, eV, aeJ, A, RT, AQ, aeI, po, yq, aeP, bP, GU, CA, PI, hL, aez, FL, NB, B, NO, dA, eX, v, yU, Kx, aeD, UE, KB, ae, zH, vT, ev, Lf, bu, t, aff, ck, kP, df, eq, hd, vc, uD, mu, d, aek, KD, Z, dr, aeb, bF, yE, afd, gZ, aef, Kj, y, hV, al, gf, Ap, aej, xE, u, gH, Qk, M, K, dF, eL, aea, UL, Ku, g, uC, AM, aer, aem, U, QF, dW, m, Uw, FW, Ol, aw, NT, aeo, ld, KL, KH, yZ, uV, aev, aeF, Pp, Ld, afc, kz, mg, lB, afj, jx, aee, j, pd, eA, J, o, er, aeZ, qv, bq, aeY, kl, Y, r, en, bN, sn, c, kB, aeM, dy, UF, LS, afa, id, aZ, bS, hF, dP, aet, oj, aeI, xs, PA, nf, gE, BG, jg, iR, FX, dR, Nq, pu, eB, ei, G, acz, afh, aei, aR, il, zQ, LC, aeC, aW, eM, aep, Qw, zv, aeA, Bm, fr, ct, qN, ai, eW, BI, cc, hw, adY, PD, h, dL, Go, SD, Lg, aeG, aeI, Nr, Fn, CM, aq, aeW, mo, RI, op, bJ, adZ, JJ, lp, bL, rG, aec, rf, hy, Kh, Ns, aT, vH, dz, adV, af, qw, iU, s, ND, oV, hf, pH, nM, yW, im, aen, lD, Fb, mC, qC, av, l, ib, aeI, aeE, pq, na, ul, afe, qt, aeq, eZ, aF, pc, bE, Lb, XO, X); #c1(AR) c2(NP_001011645) c3(androgen receptor isoform 2) c4(24654) c5(24655, 24656, 24657, 24658, 24659, 24660) c6( ) c7(gh, T, aey, ea, bO, fd, aeu, ci, YD, xy, aex, aE, lv, hu, li, nY, uY, PQ, afg, nB, aeB, lq, cx, ht, aj, ml, aex, aeI, adX, Ak, ey, ox, Dz, bl, on, yi, mR, Ln, pm, zZ, hq, dB, JQ, aA, cz, AD, q, aew, aeh, kf, cy, jj, eu, bA, bf, aek, aeS, eS, Lc, cl, pt, rR, acW, ac, iQ, bt, aag, iW, KW, jh, adW, KO, uu, sg, ad, afb, hX, vJ, lQ, wm, aeT, Ub, cW, PE, ff, afi, aeV, bz, DF, aeg, acH, aed, iu, ne, jr, Fv, if, bB, cL, bh, Ul, ip, ws, aeO, aeN, xQ, aeR, cm, qJ, Kv, AV, vA, mx, vy, in, ik, dD, gU, wu, n, oY, nA, no, Do, aes, eV, aeJ, A, RT, AQ, aeU, po, yq, aeP, bP, GU, cA, PI, hL, aez, fL, NB, B, NO, dA, eX, v, yU, Kx, aeD, UE, KB, ae, zH, vT, ev, Lf, bu, t, aff, ck, kP, df, eq, hd, vc, uD, mu, d, aek, KD, Z, dr, aeb, bF, yE, afd, gZ, aef, Kj, y, hV, al, gf, Qp, aej, xE, u, gH, Qk, M, K, dF, eL, aea, UL, Ku, g, uC, AM, aer, aem, U, QF, dW, m, Uw, FW, Ol, aw, NT, aeo, ld, KL, KH, yZ, uV, aev, aef, Pp, Ld, afc, kz, mg, lB, afj, jx, aee, j, pd, eA, J, o, er, aeZ, qv, bq, aeY, kl, Y, r, en, bN, sn, c, KB, aeM, dy, UF, LS, afa, id, aZ, bS, hF, dP, aet, oj, aeI, xs, PA, nf, gE, BG, jg, iR, FX, dR, Nq, pu, eB, ei, G, acz, afh, aei, aR, il, zQ, LC, aeC, aW, eM, aep, Qw, zv, aeA, Bm, fr, ct, qN, ai, eW, Bl, cc, hw, adY, PD, h, dL, Go, SD, Lg, aeG, aeI, Nr, Fn, CM, aq, aeW, mo, RI, op, bJ, adZ, JJ, lp, bL, rG, aec, rf, hy, Kh, Ns, aT, vH, dz, adV, af, qw, iU, s, ND, oV, hf, pH, nM, yW, im, aen, lD, Fb, mC, qC, av, l, ib, aeI, aeE, pq, na, ul, afe, qt, aeq, eZ, af, pc, bE, Lb, XO, X); #c1(ARAF) c2(NP_001243126) c3(serine/threonine-protein kinase A-Raf isoform 3) c4( ) c5(89976, 89977, 89978, 89979, 89980, 89981, 89982, 89983, 89984, 89985, 89986, 89987) c6( ) c7(iu, T, Y, aT, Ld, OL, g, [p, B, bN); #c1(ARAF) c2(NP_001645) c3(serine/threonine-protein kinase A-Raf isoform 1) c4(119690, 119691, 119692) c5(119693, 119694, 119695, 119696, 119697, 119698) c6( ) c7(iu, T, Y, aT, Ld, OL, g, Qp, B, bN); #c1(ARAP1) c2(NP_001035207) c3(arf-GAP with Rho-GAP domain, ANK repeat and PH domain-containing protein 1 isoform c) c4(32619, 32620) c5(32621, 32622, 32623, 32624, 32625, 32626, 32627, 32628, 32629, 32630, 32631, 32632, 32633, 32634, 32635, 32636, 32637) c6( ) c7(bL, aW, y, aT); #c1(ARAP1) c2(NP_001128662) c3(arf-GAP with Rho-GAP domain, ANK repeat and PH domain-containing protein 1 isoform d) c4(50489) c5(50490, 50491, 50492, 50493, 50494, 50495) c6( ) c7(bL, aW, y, aT); #c1(ARAP1) c2(NP_056057) c3(arf-GAP with Rho-GAP domain, ANK repeat and PH domain-containing protein 1 isoform a) c4(185412, 185413, 185414) c5(185415, 185416, 185417, 185418, 185419, 185420, 185421, 185422, 185423, 185424, 185425, 185426, 185427, 185428, 185429, 185430, 185431) c6( ) c7(bL, aW, y, aT); #c1(ARAP3) c2(NP_071926) c3(arf-GAP with Rho-GAP domain, ANK repeat and PH domain-containing protein 3) c4(212361) c5(212362, 212363, 212364, 212365, 212366, 212367) c6( ) c7(DS, T, aF, j, h); #c1(ARAP3) c2(XP_005268554) c3(arf-GAP with Rho-GAP domain, ANK repeat and PH domain-containing protein 3 isoform X1) c4(281894, 281895) c5(281896, 281897, 281898, 281899, 281900, 281901) c6( ) c7(DS, T, aF, j, h); #c1(ARAP3) c2(XP_005268557) c3(arf-GAP with Rho-GAP domain, ANK repeat and PH domain-containing protein 3 isoform X6) c4( ) c5(281902, 281903, 281904, 281905, 281906, 281907) c6( ) c7(DS, T, aF, j, h); #c1(ARAP3) c2(XP_006714855) c3(arf-GAP with Rho-GAP domain, ANK repeat and PH domain-containing protein 3 isoform X3) c4(288264) c5(288265, 288266, 288267, 288268, 288269, 288270) c6( ) c7(DS, T, aF, j, h); #c1(ARC) c2(NP_056008) c3(activity-regulated cytoskeleton-associated protein) c4(185097) c5(185098, 185099, 185100, 185101, 185102, 185103, 185104) c6( ) c7(aw, afk, bB, j, fr, afl, jw, q, gM, h, K, af, Y, r, cu, xw, es, sS, iA, bE, mx, R, y); #c1(ARCN1) c2(NP_001135753) c3(coatomer subunit delta isoform 2) c4( ) c5(53081, 53082, 53083, 53084, 53085, 53086) c6( ) c7(oO, bM, T, dD, am, lD, ea, fG, e, cz, n, bS, gf, ct, Jz, j, eq, cd, zo, eA, afo, afm, oz, afn, ds, XY, cU, bl, K, NM, N, aa, ly, sp, ap, afq, Z, bz, DF, lx, ou, TA, cT, ig, at, dy, fD, lC, afp, cE, ao); #c1(ARCN1) c2(NP_001646) c3(coatomer subunit delta isoform 1) c4(119699, 119700, 119701) c5(119702, 119703, 119704, 119705, 119706, 119707, 119708, 119709, 119710, 119711, 119712, 119713) c6( ) c7(oO, bM, T, dD, am, lD, ea, fG, e, cz, n, bS, gf, ct, Jz, j, eq, cd, zo, eA, afo, afm, oz, afn, ds, XY, cU, bl, K, NM, N, aa, ly, sp, ap, afq, Z, bz, DF, lx, OU, TA, cT, ig, at, dy, fD, lC, afp, cE, ao); #c1(ARCN1) c2(XP_005271599) c3(coatomer subunit delta isoform X1) c4(283501) c5(283502, 283503, 283504, 283505, 283506, 283507) c6( ) c7(oO, bM, T, dD, am, ID, ea, fG, e, cz, n, bS, gf, ct, Jz, j, eq, cd, zo, eA, afo, afm, oz, afn, ds, XY, cU, bl, K, NM, N, aa, ly, sp, ap, afq, Z, bz, DF, lx, OU, TA, cT, ig, at, dy, fD, lC, afp, cE, ao); #c1(AREG) c2(NP_001648) c3(amphiregulin preproprotein) c4(119726) c5(119727, 119728, 119729, 119730, 119731, 119732, 119733, 119734, 119735, 119736, 119737, 119738, 119739, 119740, 119741, 119742) c6(119743, 119744) c7(bM, T, aZ, fG, yk, al, iB, aA, bB, fd, hy, Fb, j, ci, pM, nE, zP, AD, q, ad, er, eA, dP, av, afr, iu, wD, aE, db, gd, aT, h, ji, lO, fb, ae, aa, zQ, d, s, aW, sp, oV, az, ff, cr, r, kt, aq, fe, kp, aF, en, g, iR, t, iK, KM, TA, gk, zr, eM, bE, gX, m, i, y, B, vi); #c1(ARF1) c2(NP_001019398) c3(ADP-ribosylation factor 1) c4(27762, 27763) c5(27764, 27765, 27766, 27767, 27768, 27769, 27770, 27771, 27772, 27773, 27774, 27775, 27776, 27777, 27778, 27779, 27780, 27781, 27782, 27783) c6( ) c7(T, jv, aW, jx, aA, aT, h, af, af, rk); #c1(ARF3) c2(NP_001650) c3(ADP-ribosylation factor 3) c4(119745, 119746, 119747, 119748) c5(119749, 119750, 119751, 119752, 119753, 119754, 119755, 119756, 119757, 119758, 119759, 119760, 119761, 119762, 119763, 119764) c6( ) c7(T, vT, aW, aT, K, afs, cm, dR); #c1(ARF3) c2(XP_006719454) c3(ADP-ribosylation factor 3 isoform X2) c4(292473) c5(292474, 292475, 292476, 292477, 292478, 292479, 292480, 292481, 292482, 292483, 292484, 292485, 292486, 292487, 292488, 292489, 292490, 292491) c6( ) c7(T, vT, aW, aT, K, afs, cm, dR); #c1(ARF4) c2(NP_001651) c3(ADP-ribosylation factor 4) c4(119765, 119766, 119767) c5(119768, 119769, 119770, 119771, 119772, 119773, 119774, 119775, 119776, 119777, 119778, 119779, 119780, 119781, 119782, 119783, 119784, 119785) c6( ) c7(aW, T, d, K, aT); #c1(ARF6) c2(NP_001654) c3(ADP-ribosylation factor 6) c4(119793) c5(119794, 119795, 119796, 119797, 119798, 119799) c6( ) c7(T, jv, aW, Y, aT, aft, cy, By, r, aq, eA, h, pz, B, ek, afu); #c1(ARFGAP1) c2(NP_001268411) c3(ADP-ribosylation factor GTPase-activating protein 1 isoform c) c4(99851) c5(99852, 99853, 99854, 99855, 99856, 99857, 99858, 99859, 99860, 99861, 99862, 99863, 99864) c6( ) c7(aR, r); #c1(ARFGAP1) c2(NP_001268412) c3(ADP-ribosylation factor GTPase-activating protein 1 isoform d) c4(99865) c5(99866, 99867, 99868, 99869, 99870, 99871) c6( ) c7(aR, r); #c1(ARFGAP1) c2(NP_001268413) c3(ADP-ribosylation factor GTPase-activating protein 1 isoform e) c4(99872) c5(99873, 99874, 99875, 99876, 99877, 99878) c6( ) c7(aR, r); #c1(ARFGAP1) c2(NP_060679) c3(ADP-ribosylation factor GTPase-activating protein 1 isoform a) c4(197129, 197130) c5(197131, 197132, 197133, 197134, 197135, 197136) c6( ) c7(aR, r); #c1(ARFGAP1) c2(NP_783202) c3(ADP-ribosylation factor GTPase-activating protein 1 isoform b) c4(251572) c5(251573, 251574, 251575, 251576, 251577, 251578) c6( ) c7(aR, r); #c1(ARFGAP1) c2(XP_006723886) c3(ADP-ribosylation factor GTPase-activating protein 1 isoform X2) c4(295431, 295432) c5( ) c6( ) c7(aR, r); #c1(ARFGAP1) c2(XP_011527204) c3(ADP-ribosylation factor GTPase-activating protein 1 isoform X3) c4( ) c5(321742, 321743, 321744, 321745, 321746, 321747, 321748, 321749, 321750, 321751, 321752, 321753, 321754) c6( ) c7(aR, r); #c1(ARFGAP2) c2(NP_001229761) c3(ADP-ribosylation factor GTPase-activating protein 2 isoform 2) c4(85/77) c5(85178, 85179, 85180, 85181, 85182, 85183, 85184, 85185, 85186, 85187, 85188, 85189, 85190, 85191, 85192, 85193, 85194, 85195) c6( ) c7(aA); #c1(ARFGAP2) c2(NP_115765) c3(ADP-ribosylation factor GTPase-activating protein 2 isoform 1) c4(223859) c5(223860, 223861, 223862, 223863, 223864, 223865, 223866) c6( ) c7(aA); #c1(ARFGAP2) c2(XP_005253225) c3(ADP-ribosylation factor GTPase-activating protein 2 isoform X4) c4(272286) c5( ) c6( ) c7(aA); #c1(ARFGAP2) c2(XP_011518707) c3(ADP-ribosylation factor GTPase-activating protein 2 isoform X5) c4(313126) c5( ) c6( ) c7(aA); #c1(ARFGAP3) c2(NP_001135765) c3(ADP-ribosylation factor GTPase-activating protein 3 isoform 2) c4(53165, 53166, 53167) c5(53168, 53169, 53170, 53171, 53172, 53173, 53174, 53175, 53176, 53177, 53178, 53179, 53180, 53181, 53182, 53183, 53184, 53185) c6( ) c7(ae, ad, r); #c1(ARFGAP3) c2(NP_055385) c3(ADP-ribosylation factor GTPase-activating protein 3 isoform 1) c4(180712) c5(180713, 180714, 180715, 180716, 180717, 180718) c6( ) c7(ae, ad, r); #c1(ARFGAP3) c2(XP_005261582) c3(ADP-ribosylation factor GTPase-activating protein 3 isoform X1) c4(277497) c5(277498, 277499, 277500, 277501, 277502, 277503) c6( ) c7(ae, ad, r); #c1(ARFGEF1) c2(NP_006412) c3(brefeldin A-inhibited guanine nucleotide-exchange protein 1) c4(164809) c5(164810, 164811, 164812, 164813, 164814, 164815) c6( ) c7(bM, T, aW, afi, aT, afv, ey); #c1(ARFGEF2) c2(NP_006411) c3(brefeldin A-inhibited guanine nucleotide-exchange protein 2) c4(164795, 164796) c5(164797, 164798, 164799, 164800, 164801, 164802, 164803, 164804, 164805, 164806, 164807, 164808) c6( ) c7(afy, rf, afw, afz, bK, ey, QK, afx, fh, cC); #c1(ARFGEF3) c2(NP_065073) c3(brefeldin A-inhibited guanine nucleotide-exchange protein 3) c4(203633, 203634) c5(203635, 203636, 203637, 203638, 203639, 203640, 203641, 203642, 203643, 203644, 203645, 203646) c6( ) c7(aW, aT); #c1(ARFIP1) c2(NP_001020766) c3(arfaptin-1 isoform 1) c4(29010) c5(29011, 29012, 29013, 29014, 29015, 29016, 29017, 29018, 29019, 29020) c6( ) c7(eG, jN); #c1(ARFIP1) c2(NP_055262) c3(arfaptin-1 isoform 2) c4(180130) c5(180131, 180132, 180133, 180134, 180135, 180136, 180137, 180138, 180139, 180140) c6( ) c7(eG, jN); #c1(ARFRP1) c2(NP_001128230) c3(ADP-ribosylation factor-related protein 1 isoform b) c4(49737, 49738, 49739) c5(49740, 49741, 49742, 49743, 49744, 49745) c6( ) c7(ck, BC, BD, oV, fd, aq, K, hF, B); #c1(ARFRP1) c2(NP_001254474) c3(ADP-ribosylation factor-related protein 1 isoform d) c4( ) c5(94372, 94373, 94374, 94375, 94376, 94377, 94378, 94379, 94380, 94381, 94382, 94383) c6( ) c7(ck, BC, BD, oV, fd, aq, K, hF, B); #c1(ARFRP1) c2(NP_001254475) c3(ADP-ribosylation factor-related protein 1 isoform e) c4( ) c5(94384, 94385, 94386, 94387, 94388, 94389) c6( ) c7(ck, BC, BD, oV, fd, aq, K, hF, B); #c1(ARFRP1) c2(NP_001254477) c3(ADP-ribosylation factor-related protein 1 isoform a) c4(94390, 94391) c5(94392, 94393, 94394, 94395, 94396, 94397, 94398, 94399, 94400, 94401) c6( ) c7(ck, BC, BD, oV, fd, aq, K, hF, B); #c1(ARG1) c2(NP_000036) c3(arginase-1 isoform 2) c4(375, 376) c5(377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475) c6(476) c7(bM, T, dD, Dw, afA, ea, fG, e, ht, jx, aw, aA, cR, cz, v, j, eq, bh, pM, fr, afD, qW, ad, ady, jz, oj, Lv, lG, Ds, cy, bq, hu, ae, rL, afC, Y, EU, hl, cr, afB, fQ, dq, hJ, LB, iO, D, fT, eM, z, Vy, Ny, E, lC, B, y); #c1(ARG1) c2(NP_001231367) c3(arginase-1 isoform 1) c4(87252) c5(87253, 87254, 87255, 87256, 87257, 87258) c6( ) c7(bM, T, dD, Dw, afA, ea, fG, e, ht, jx, aw, aA, CR, cz, v, j, eq, bh, pM, fr, afD, qW, ad, ady, jz, oj, Lv, lG, Ds, cy, bq, hu, ae, rL, afC, Y, EU, hl, cr, afB, fQ, dq, hJ, LB, iO, D, fT, eM, z, Vy, Ny, E, IC, B, y); #c1(ARGLU1) c2(NP_060481) c3(arginine and glutamate-rich protein 1) c4(196166, 196167, 196168) c5(196169, 196170, 196171, 196172, 196173, 196174, 196175, 196176, 196177, 196178, 196179, 196180, 196181, 196182, 196183, 196184, 196185, 196186, 196187) c6( ) c7(Z, aW, aj, aT); #c1(ARHGAP1) c2(XP_011518397) c3(rho GTPase-activating protein 1 isoform X1) c4(312363, 312364, 312365) c5(312366, 312367, 312368, 312369, 312370, 312371, 312372, 312373, 312374, 312375, 312376, 312377, 312378, 312379, 312380, 312381, 312382, 312383) c6( ) c7(iu, T, aE, aW, KW, aT, nf, cz, aq, K, nA, iR, yj, ae, B, ad); #c1(ARHGAP10) c2(NP_078881) c3(rho GTPase-activating protein 10) c4(215876) c5(215877, 215878, 215879, 215880, 215881, 215882) c6( ) c7(aW, oe, EQ, aT); #c1(ARHGAP10) c2(XP_005263272) c3(rho GTPase-activating protein 10 isoform X1) c4(278380) c5(278381, 278382, 278383, 278384, 278385, 278386) c6( ) c7(aW, oe, EQ, aT); #c1(ARHGAP11A) c2(NP_001273408) c3(rho GTPase-activating protein (IA isoform 3) c4(104846) c5(104847, 104848, 104849, 104850, 104851) c6( ) c7(bB, T, v, ey, K); #c1(ARHGAP11A) c2(NP_055598) c3(rho GTPase-activating protein (IA isoform 1) c4(182177) c5(182178, 182179, 182180, 182181, 182182, 182183, 182184) c6( ) c7(bB, T, v, ey, K); #c1(ARHGAP11A) c2(NP_955389) c3(rho GTPase-activating protein (A isoform 2) c4(261660) c5(261661, 261662, 261663, 261664, 261665, 261666) c6( ) c7(bB, T, v, ey, K); #c1(ARHGAP11B) c2(NP_001034930) c3(rho GTPase-activating protein 11B) c4(32397) c5(32398, 32399, 32400, 32401, 32402, 32403, 32404, 32405, 32406, 32407) c6( ) c7(ac); #c1(ARHGAP15) c2(NP_060930) c3(rho GTPase-activating protein 15) c4(198749) c5(198750, 198751, 198752, 198753, 198754, 198755) c6( ) c7(cd, K); #c1(ARHGAP18) c2(NP_277050) c3(rho GTPase-activating protein 18) c4(228814) c5(228815, 228816, 228817, 228818, 228819, 228820, 228821) c6( ) c7(Lu, c, dL, yH, K, eL, af); #c1(ARHGAP18) c2(XP_005267269) c3(rho GTPase-activating protein 18 isoform X1) c4(280843) c5(280844, 280845, 280846, 280847, 280848) c6( ) c7(Lu, c, dL, yH, K, el, af); #c1(ARHGAP20) c2(NP_001245344) c3(rho GTPase-activating protein 20 isoform 2) c4(93165) c5(93166, 93167, 93168, 93169, 93170, 93171) c6( ) c7(cj); #c1(ARHGAP20) c2(NP_001245345) c3(rho GTPase-activating protein 20 isoform 3) c4(93/72) c5(93173, 93174, 93175, 93176, 93177, 93178, 93179, 93180, 93181, 93182) c6( ) c7(cj); #c1(ARHGAP20) c2(XP_005271685) c3(rho GTPase-activating protein 20 isoform X2) c4(283528) c5(283529, 283530, 283531, 283532, 283533) c6( ) c7(cj); #c1(ARHGAP20) c2(XP_006718956) c3(rho GTPase-activating protein 20 isoform X1) c4(292119, 292120) c5(292121, 292122, 292123, 292124, 292125, 292126, 292127, 292128, 292129, 292130, 292131, 292132, 292133, 292134, 292135, 292136, 292137) c6( ) c7(cj); #c1(ARHGAP21) c2(XP_005252599) c3(rho GTPase-activating protein 21 isoform X1) c4(271916, 271917) c5(271918, 271919, 271920, 271921, 271922, 271923, 271924, 271925, 271926, 271927, 271928, 271929, 271930) c6( ) c7(aW, d, aT); #c1(ARHGAP21) c2(XP_005252601) c3(rho GTPase-activating protein 21 isoform X4) c4(271931, 271932, 271933) c5(271934, 271935, 271936, 271937, 271938, 271939, 271940, 271941, 271942, 271943, 271944, 271945, 271946, 271947, 271948, 271949, 271950, 271951, 271952, 271953, 271954, 271955) c6( ) c7(aW, d, aT); #c1(ARHGAP21) c2(XP_011517906) c3(rho GTPase-activating protein 21 isoform X2) c4( ) c5(311830, 311831, 311832, 311833, 311834, 311835) c6( ) c7(aW, d, aT); #c1(ARHGAP21) c2(XP_011517907) c3(rho GTPase-activating protein 21 isoform X3) c4(311836) c5( ) c6( ) c7(aW, d, aT); #c1(ARHGAP21) c2(XP_011517908) c3(rho GTPase-activating protein 21 isoform X5) c4( ) c5(311837, 311838, 311839, 311840, 311841, 311842) c6( ) c7(aW, d, aT); #c1(ARHGAP21) c2(XP_011517909) c3(rho GTPase-activating protein 21 isoform X6) c4(311843, 311844, 311845) c5(311846, 311847, 311848, 311849, 311850, 311851, 311852) c6( ) c7(aW, d, aT); #c1(ARHGAP22) c2(NP_001242953) c3(rho GTPase-activating protein 22 isoform 1) c4(89432) c5(89433, 89434, 89435, 89436, 89437, 89438, 89439, 89440, 89441, 89442, 89443, 89444) c6( ) c7(oe, vy, aj, ey, Gp, J); #c1(ARHGAP22) c2(NP_001242954) c3(rho GTPase-activating protein 22 isoform 2) c4(89445) c5(89446, 89447, 89448, 89449, 89450, 89451) c6( ) c7(oe, vy, aj, ey, Gp, J); #c1(ARHGAP22) c2(NP_001242955) c3(rho GTPase-activating protein 22 isoform 4) c4(89452) c5(89453, 89454, 89455, 89456, 89457, 89458) c6( ) c7(oe, vy, aj, ey, Gp, J); #c1(ARHGAP22) c2(NP_067049) c3(rho GTPase-activating protein 22 isoform 3) c4(208731) c5(208732, 208733, 208734, 208735, 208736, 208737) c6( ) c7(oe, vy, aj, ey, Gp, J); #c1(ARHGAP22) c2(XP_011538309) c3(rho GTPase-activating protein 22 isoform X5) c4(335994) c5( ) c6( ) c7(oe, vy, aj, ey, Gp, J); #c1(ARHGAP22) c2(XP_011538314) c3(rho GTPase-activating protein 22 isoform X10) c4( ) c5(335995) c6( ) c7(oe, vy, aj, ey, Gp, J); #c1(ARHGAP23) c2(NP_001186346) c3(rho GTPase-activating protein 23) c4(77500) c5(77501, 77502, 77503, 77504, 77505, 77506) c6( ) c7(kl); #c1(ARHGAP23) c2(XP_006722054) c3(rho GTPase-activating protein 23 isoform X2) [4( ) c5(294560) c6( ) c7(kl); #c1(ARHGAP23) c2(XP_006722057) c3(rho GTPase-activating protein 23 isoform X8) c4(294561) c5(294562, 294563, 294564, 294565, 294566, 294567) c6( ) c7(kl); #c1(ARHGAP23) c2(XP_011523376) c3(rho GTPase-activating protein 23 isoform X3) c4(317907) c5(317908, 317909, 317910, 317911, 317912) c6( ) c7(kl); #c1(ARHGAP23) c2(XP_011523379) c3(rho GTPase-activating protein 23 isoform X9) c4( ) c5(317913) c6( ) c7(kl); #c1(ARHGAP24) c2(NP_112595) c3(rho GTPase-activating protein 24 isoform 2) c4(221526) c5(221527, 221528, 221529, 221530, 221531, 221532) c6( ) c7(gh, T, os, ea, if, al, EQ, fd, bB, ci, Hr, ip, x, aE, Ma, K, hu, eL, JI, aW, g, as, eF, ir, cm, bw, eM, nB, iA, iT, cj, iS, k, m, dD, ht, NT, n, afE, fr, qN, br, co, jz, hn, dL, hB, MD, GA, aq, YE, bP, dB, eD, hL, pV, eo, it, B, fM, jx, aA, V, q, eA, qK, aT, j, ae, af, jf, qw, s, iU, Y, r, en, hf, cl, t, ik, ck, sk, cT, gX, dy, bt, aD, iW, jh, oZ, OH, eq, hF, Fc, ad, afb, hX, iC, hd, io, vw, yF, d, aff, ff, eH, aF, bz, Hp, es, gk, bE, Km, aS, adg, iu); #c1(ARHGAP24) c2(XP_005263320) c3(rho GTPase-activating protein 24 isoform X2) c4(278415, 278416) c5(278417, 278418, 278419, 278420, 278421, 278422, 278423, 278424, 278425, 278426, 278427, 278428, 278429, 278430) c6( ) c7(gh, T, os, ea, if, al, EQ, fd, bB, ci, Hr, ip, x, aE, Ma, K, hu, eL, JI, aW, g, as, eF, ir, cm, bw, eM, nB, iA, iT, cj, iS, k, m, dD, ht, NT, n, afE, fr, qN, br, co, jz, hn, dL, hB, MD, GA, aq, YE, bP, dB, eD, hL, pV, eo, it, B, fM, jx, aA, V, q, eA, qK, aT, j, ae, af, jf, qw, s, iU, Y, r, en, hf, cl, t, iK, ck, sk, cT, gX, dy, bt, aD, iW, jh, oZ, OH, eq, hF, Fc, ad, afb, hX, iC, hd, io, vw, yF, d, aff, ff, eH, aF, bz, Hp, es, gk, bE, Km, aS, adg, iu); #c1(ARHGAP24) c2(XP_011530602) c3(rho GTPase-activating protein 24 isoform X1) c4(326503, 326504) c5(326505, 326506, 326507, 326508, 326509, 326510, 326511, 326512, 326513, 326514) c6( ) c7(gh, T, os, ea, if, al, EQ, fd, bB, ci, Hr, ip, x, aE, Ma, K, hu, eL, JI, aW, g, as, eF, ir, cm, bw, eM, nB, iA, iT, cj, iS, k, m, dD, ht, NT, n, afE, fr, qN, br, co, jz, hn, dL, hB, MD, GA, aq, YE, bP, dB, eD, hL, pV, eo, it, B, fM, jx, aA, V, q, eA, qK, aT, j, ae, af, jf, qw, s, iU, Y, r, en, hf, cl, t, iK, ck, sk, cT, gX, dy, bt, aD, iW, jh, oZ, OH, eq, hF, Fc, ad, afb, hX, iC, hd, io, vw, yF, d, aff, ff, eH, aF, bz, Hp, es, gk, bE, Km, aS, adg, iu); #c1(ARHGAP25) c2(NP_001007232) c3(rho GTPase-activating protein 25 isoform a) c4(22579) c5(22580, 22581, 22582, 22583, 22584, 22585, 22586, 22587) c6( ) c7(EQ); #c1(ARHGAP25) c2(NP_001159748) c3(rho GTPase-activating protein 25 isoform c) c4(64665, 64666, 64667, 64668) c5(64669, 64670, 64671, 64672, 64673, 64674, 64675, 64676, 64677, 64678, 64679, 64680, 64681, 64682, 64683, 64684, 64685, 64686, 64687, 64688, 64689, 64690, 64691, 64692) c6( ) c7(EQ); #c1(ARHGAP25) c2(NP_001159749) c3(rho GTPase-activating protein 25 isoform d) c4(64693) c5(64694, 64695, 64696, 64697, 64698, 64699) c6( ) c7(EQ); #c1(ARHGAP25) c2(NP_055697) c3(rho GTPase-activating protein 25 isoform b) c4(182847) c5(182848, 182849, 182850, 182851, 182852, 182853) c6( ) c7(EQ); #c1(ARHGAP25) c2(XP_005264732) c3(rho GTPase-activating protein 25 isoform X1) c4(279220) c5(279221, 279222, 279223, 279224, 279225, 279226) c6( ) c7(EQ); #c1(ARHGAP25) c2(XP_011531510) c3(rho GTPase-activating protein 25 isoform X2) c4(327662) c5(327663, 327664, 327665, 327666, 327667, 327668, 327669, 327670) c6( ) c7(EQ); #c1(ARHGAP26) c2(NP_001129080) c3(rho GTPase-activating protein 26 isoform b) c4(50757, 50758) c5(50759, 50760, 50761, 50762, 50763, 50764, 50765, 50766, 50767, 50768, 50769, 50770) c6( ) c7(bL, ck, T, pq, iT, iU, iZ, bw, nA, cm, bt, br); #c1(ARHGAP26) c2(NP_055886) c3(rho GTPase-activating protein 26 isoform a) c4(184167, 184168) c5(184169, 184/70, 184171, 184172, 184/73, 184/74) c6( ) c7(bL, ck, T, pq, iT, iU, iZ, bw, nA, cm, bt, br); #c1(ARHGAP26) c2(XP_005268459) c3(rho GTPase-activating protein 26 isoform X5) c4(281822) c5(281823, 281824, 281825, 281826, 281827, 281828, 281829, 281830, 281831, 281832, 281833, 281834) c6( ) c7(bL, ck, T, pq, iT, iU, iZ, bw, nA, cm, bt, br); #c1(ARHGAP27) c2(NP_777579) c3(rho GTPase-activating protein 27 isoform c) c4(251234, 251235) c5(251236, 251237, 251238, 251239, 251240, 251241, 251242, 251243, 251244, 251245, 251246, 251247, 251248, 251249, 251250, 251251) c6( ) c7(m); #c1(ARHGAP27) c2(NP_954976) c3(rho GTPase-activating protein 27 isoform a) c4(261496, 261497) c5( ) c6( ) c7(m); #c1(ARHGAP27) c2(XP_005257185) c3(rho GTPase-activating protein 27 isoform X8) c4(274733) c5(274734, 274735, 274736, 274737, 274738, 274739) c6( ) c7(m); #c1(ARHGAP27) c2(XP_006721808) c3(rho GTPase-activating protein 27 isoform X3) c4(294279, 294280) c5(294281, 294282, 294283, 294284, 294285, 294286, 294287, 294288, 294289, 294290, 294291, 294292, 294293, 294294, 294295, 294296, 294297, 294298, 294299, 294300, 294301, 294302, 294303, 294304, 294305, 294306, 294307, 294308, 294309, 294310, 294311, 294312, 294313, 294314) c6( ) c7(m); #c1(ARHGAP27) c2(XP_006725322) c3(rho GTPase-activating protein 27 isoform X3) c4(296762) c5(296763, 296764, 296765, 296766, 296767, 296768) c6( ) c7(m); #c1(ARHGAP27) c2(XP_011522774) c3(rho GTPase-activating protein 27 isoform X7) c4( ) c5(317301) c6( ) c7(m); #c1(ARHGAP28) c2(NP_001010000) c3(rho GTPase-activating protein 28) c4(23815) c5(23816, 23817, 23818, 23819, 23820, 23821, 23822, 23823) c6( ) c7(dP, j); #c1(ARHGAP28) c2(XP_005258201) c3(rho GTPase-activating protein 28 isoform X1) c4( ) c5(275542, 275543) c6( ) c7(dP, j); #c1(ARHGAP28) c2(XP_005258202) c3(rho GTPase-activating protein 28 isoform X2) c4(275544, 275545, 275546, 275547, 275548, 275549) c5(275550, 275551, 275552, 275553, 275554, 275555, 275556, 275557, 275558, 275559, 275560, 275561, 275562, 275563, 275564, 275565, 275566, 275567, 275568, 275569, 275570, 275571, 275572, 275573, 275574, 275575, 275576, 275577, 275578, 275579) c6( ) c7(dP, j); #c1(ARHGAP28) c2(XP_005258203) c3(rho GTPase-activating protein 28 isoform X3) c4( ) c5(275580, 275581, 275582, 275583, 275584, 275585) c6( ) c7(dP, j); #c1(ARHGAP30) c2(NP_001020769) c3(rho GTPase-activating protein 30 isoform 1) c4(29021, 29022) c5(29023, 29024, 29025, 29026, 29027, 29028) c6( ) c7(kl); #c1(ARHGAP30) c2(NP_859071) c3(rho GTPase-activating protein 30 isoform 2) c4(255488) c5(255489, 255490, 255491, 255492, 255493, 255494, 255495, 255496, 255497, 255498, 255499, 255500) c6( ) c7(kl); #c1(ARHGAP31) c2(NP_065805) c3(rho GTPase-activating protein 31) c4(205617, 205618) c5(205619, 205620, 205621, 205622, 205623, 205624, 205625, 205626, 205627, 205628, 205629, 205630) c6( ) c7(afG, aW, hp, j, aT); #c1(ARHGAP32) c2(NP_001136157) c3(rho GTPase-activating protein 32 isoform 1) c4(54456, 54457, 54458) c5(54459, 54460, 54461, 54462, 54463, 54464, 54465, 54466, 54467, 54468, 54469, 54470, 54471) c6( ) c7(K); #c1(ARHGAP32) c2(XP_011541376) c3(rho GTPase-activating protein 32 isoform X4) c4(339997) c5(339998, 339999, 340000, 340001, 340002) c6( ) c7(K); #c1(ARHGAP32) c2(XP_011541377) c3(rho GTPase-activating protein 32 isoform X5) c4( ) c5(340003, 340004, 340005, 340006, 340007, 340008) c6( ) c7(K); #c1(ARHGAP35) c2(NP_004482) c3(rho GTPase-activating protein 35) c4(146778, 146779, 146780) c5(146781, 146782, 146783, 146784, 146785, 146786, 146787, 146788, 146789, 146790, 146791, 146792, 146793) c6( ) c7(Y, r, eA, q, ci, Fc, ai); #c1(ARHGAP35) c2(XP_011525175) c3(rho GTPase-activating protein 35 isoform X1) c4( ) c5(319619, 319620, 319621, 319622, 319623, 319624) c6( ) c7(Y, r, eA, q, ci, Fc, ai); #c1(ARHGAP4) c2(NP_001158213) c3(rho GTPase-activating protein 4 isoform 1) c4(63403) c5(63404, 63405, 63406, 63407, 63408, 63409, 63410, 63411, 63412, 63413, 63414, 63415) c6( ) c7(h, yj, adB, j); #c1(ARHGAP4) c2(NP_001657) c3(rho GTPase-activating protein 4 isoform 2) c4(119800, 119801, 119802, 119803) c5(119804, 119805, 119806, 119807, 119808, 119809, 119810, 119811, 119812, 119813, 119814, 119815, 119816, 119817, 119818, 119819, 119820, (19821) c6( ) c7(h, yj, adB, j); #c1(ARHGAP42) c2(NP_689645) c3(rho GTPase-activating protein 42) c4(243418) c5(243419, 243420, 243421, 243422, 243423, 243424) c6( ) c7(oe, j, cz); #c1(ARHGAP5) c2(NP_001164) c3(rho GTPase-activating protein 5 isoform b) c4(67260, 67261) c5( ) c6( ) c7(av, bz, B); #c1(ARHGAP5) c2(XP_005267692) c3(rho GTPase-activating protein 5 isoform X1) c4(281201, 281202, 281203, 281204, 281205) c5(281206, 281207, 281208, 281209, 281210, 281211, 281212, 281213, 281214, 281215, 281216, 281217, 281218, 281219, 281220, 281221) c6( ) c7(av, bz, B); #c1(ARHGAP6) c2(NP_001274171) c3(rho GTPase-activating protein 6 isoform 6) c4( ) c5(106101) c6( ) c7(Ud, afH, cz); #c1(ARHGAP6) c2(NP_006116) c3(rho GTPase-activating protein 6 isoform 3) c4(162188, 162189) c5(162190, 162191, 162192, 162193, 162194, 162195, 162196, 162197, 162198, 162199, 162200, 162201, 162202, 162203, 162204, 162205, 162206, 162207, 162208, 162209, 162210, 162211, 162212, 162213) c6( ) c7(Ud, afH, cz); #c1(ARHGAP6) c2(NP_038267) c3(rho GTPase-activating protein 6 isoform 4) c4(176341) c5(176342, 176343, 176344, 176345, 176346, 176347, 176348, 176349, 176350) c6( ) c7(Ud, afH, cz); #c1(ARHGAP6) c2(NP_038286) c3(rho GTPase-activating protein 6 isoform 1) c4(176351, 176352, 176353, 176354) c5(176355, 176356, 176357, 176358, 176359, 176360, 176361, 176362, 176363, 176364, 176365, 176366, 176367, 176368) c6( ) c7(Ud, afH, cz): #c1(ARHGAP9) c2(NP_001073625) c3(rho GTPase-activating protein 9 isoform 3) c4(36091, 36092, 36093) c5(36094, 36095, 36096, 36097, 36098, 36099, 36100, 36101, 36102, 36103, 36104, 36105, 36106, 36107, 36108) c6( ) c7(cR, afl, mw, lu); #c1(ARHGAP9) c2(NP_001073626) c3(rho GTPase-activating protein 9 isoform 2) c4( ) c5(36109, 36110, 36111, 36112, 36113, 36114, 36115) c6( ) c7(cR, afl, mw, lu); #c1(ARHGAP9) c2(NP_115885) c3(rho GTPase-activating protein 9 isoform) c4(224503, 224504, 224505, 224506, 224507) c5(224508, 224509, 224510, 224511, 224512, 224513, 224514, 224515, 224516, 224517, 224518, 224519, 224520, 224521) c6( ) c7(cR, afl, mw, lu); #c1(ARHGAP9) c2(XP_005269142) c3(rho GTPase-activating protein 9 isoform X8) c4( ) c5(282358, 282359, 282360, 282361, 282362, 282363) c6( ) c7(cR, afl, mw, lu); #c1(ARHGAP9) c2(XP_006719623) c3(rho GTPase-activating protein 9 isoform X4) c4(292712, 292713, 292714) c5(292715, 292716, 292717, 292718, 292719, 292720, 292721, 292722, 292723, 292724, 292725, 292726) c6( ) c7(cR, afl, mw, lu); #c1(ARHGAP9) c2(XP_011536965) c3(rho GTPase-activating protein 9 isoform X11) c4( ) c5(334650, 334651, 334652, 334653, 334654, 334655, 334656, 334657, 334658, 334659, 334660, 334661, 334662) c6( ) c7(cR, afl, mw, lu); #c1(ARHGD1A) c2(NP_001288171) c3(rho GDP-dissociation inhibitor 1 isoform d) c4( ) c5(113678, 113679, 113680, 113681) c6( ) c7(d, aW, aT, cz, n, afk, h, ah, afL, ci, iV, Fc, afJ); #c1(ARHGD1A) c2(NP_004300) c3(rho GDP-dissociation inhibitor 1 isoform a) c4(144973, 144974) c5(144975, 144976, 144977, 144978, 144979, 144980, 144981, 144982, 144983, 144984, 144985, 144986, 144987, 144988, 144989) c6( ) c7(d, aW, aT, cz, n, afK, h, ah, afL, ci, iV, Fc, afJ); #c1(ARHGD1B) c2(XP_011518968) c3(rho GDP-dissociation inhibitor 2 isoform X1) c4(313392, 313393, 313394, 313395) c5(313396, 313397, 313398, 313399, 313400, 313401, 313402, 313403, 313404, 313405, 313406, 313407, 313408, 313409, 313410, 313411, 313412, 313413, 313414, 313415, 313416, 313417, 313418, 313419, 313420, 313421, 313422, 313423, 313424, 313425, 313426, 313427) c6( ) c7(T, ex, aA, at, h, afK, dL, aF, eV, eL, q, ad); #c1(ARHGEF1) c2(NP_945328) c3(rho guanine nucleotide exchange factor 1 isoform 3) c4(260829) c5(260830, 260831, 260832, 260833, 260834, 260835) c6( ) c7(ck, T, afN, Qy, bP, iU, aW, afM, aT, K, g, bt); #c1(ARHGEF1) c2(NP_945353) c3(rho guanine nucleotide exchange factor 1 isoform 1) c4(260879, 260880) c5(260881, 260882, 260883, 260884, 260885, 260886, 260887, 260888, 260889, 260890, 260891, 260892) c6( ) c7(ck, T, afN, Qy, bP, iU, aW, afM, aT, K, g, bt); #c1(ARHGEF1) c2(XP_005259446) c3(rho guanine nucleotide exchange factor 1 isoform X4) c4(276096) c5( ) c6( ) c7(ck, T, afN, Qy, bP, iU, aW, afM, aT, K, g, bt); #c1(ARHGEF1) c2(XP_005259447) c3(rho guanine nucleotide exchange factor 1 isoform X6) c4(276097) c5(276098, 276099, 276100, 276101, 276102, 276103) c6( ) c7(ck, T, afN, Qy, bP, iU, aW, afM, aT, K, g, bt); #c1(ARHGEF1) c2(XP_006723526) c3(rho guanine nucleotide exchange factor 1 isoform X7) c4( ) c5(295228, 295229, 295230, 295231, 295232, 295233) c6( ) c7(ck, T, afN, Qy, bP, iU, aW, afM, aT, K, g, bt); #c1(ARHGEF10) c2(NP_055444) c3(rho guanine nucleotide exchange factor 10) c4(181173) c5(181174, 181175, 181176, 181177, 181178) c6( ) c7(afP, afO, EQ, bU); #c1(ARHGEF1D) c2(XP_006725170) c3(rho guanine nucleotide exchange factor 10 isoform X5) c4(296702) c5( ) c6( ) c7(afP, afO, EQ, bU); #c1(ARHGEF10) c2(XP_011544865) c3(rho guanine nucleotide exchange factor 10 isoform X1) c4(343866) c5( ) c6( ) c7(afP, afO, EQ, bu); #c1(ARHGEF10L) c2(XP_011539993) c3(rho guanine nucleotide exchange factor 10-like protein isoform X4) c4(338313) c5( ) c6( ) c7(hk, Y, afQ, ff, en); #c1(ARHGEF10L) c2(XP_011539996) c3(rho guanine nucleotide exchange factor 10-like protein isoform X7) c4( ) c5(338314) c6( ) c7(hk, Y, afQ, ff, en); #c1(ARHGEF10L) c2(XP_011539997) c3(rho guanine nucleotide exchange factor 10-like protein isoform X10) c4( ) c5(338315, 338316, 338317, 338318, 338319) c6( ) c7(hk, Y, afQ, ff, en); #c1(ARHGEF11) c2(NP_055599) c3(rho guanine nucleotide exchange factor II isoform 1) c4(182185) c5(182186, 182187, 182188, 182189, 182190, 182191) c6( ) c7(bL, av, T, aT, jy, mf, dy, bz, us); #c1(ARHGEF11) c2(NP_937879) c3(rho guanine nucleotide exchange factor 11 isoform 2) c4(259381) c5(259382, 259383, 259384, 259385, 259386, 259387) c6( ) c7(bL, av, T, aT, jy, mf, dy, bz, us); #c1(ARHGEF11) c2(XP_011508492) c3(rho guanine nucleotide exchange factor 11 isoform X15) c4(299211) c5(299212, 299213, 299214, 299215, 299216, 299217) c6( ) c7(bL, av, T, aT, jy, mf, dy, bz, us); #c1(ARHGEF12) c2(NP_001185594) c3(rho guanine nucleotide exchange factor 12 isoform 2) c4(75972) c5(75973, 75974, 75975, 75976, 75977, 75978) c6( ) c7(bL, ck, aW, ff, r, aT, ei, en, bt, ae, q, ad); #c1(ARHGEF12) c2(NP_056128) c3(rho guanine nucleotide exchange factor 12 isoform 1) c4(185808) c5(185809, 185810, 185811, 185812, 185813, 185814) c6( ) c7(bL, ck, aW, ff, r, aT, ei, en, bt, ae, q, ad); #c1(ARHGEF15) c2(NP_776089) c3(rho guanine nucleotide exchange factor 15) c4(250845, 250846) c5(250847, 250848, 250849, 250850, 250851, 250852, 250853, 250854, 250855, 250856, 250857, 250858, 250859, 250860, 250861, 250862) c6( ) c7(ha); #c1(ARHGEF16) c2(XP_011539555) c3(rho guanine nucleotide exchange factor 16 isoform X1) c4(337776, 337777) c5(337778, 337779, 337780, 337781, 337782, 337783, 337784, 337785, 337786, 337787, 337788, 337789) c6( ) c7(Fc); #c1(ARHGEF17) c2(NP_055601) c3(rho guanine nucleotide exchange factor 17) c4(182192) c5(182193, 182194, 182195, 182196, 182197, 182198) c6( ) c7(bB, bE, Y); #c1(ARHGEF2) c2(NP_001155855) c3(rho guanine nucleotide exchange factor 2 isoform 1) c4(61588, 61589) c5(61590, 61591, 61592, 61593, 61594, 61595, 61596, 61597, 61598, 61599, 61600, 61601) c6( ) c7(bM, T, jv, aC, yS, ha, Hn, bS, nA, V, q, ad, eA, afR, dV, aT, cU, h, afU, K, bU, afx, ae, A, aW, Y, afS, cr, r, eZ, fe, g, eE, cm, nC, bz, dr, bN, ck, bF, Jr, z, HK, eo, yA, fD, afT, y, bt, B); #c1(ARHGEF2) c2(NP_001155856) c3(rho guanine nucleotide exchange factor 2 isoform 2) c4(61602) c5(61603, 61604, 61605, 61606, 61607, 61608, 61609, 61610, 61611, 61612, 61613, 61614, 61615, 61616) c6( ) c7(bM, T, jv, aC, yS, ha, Hn, bS, nA, V, q, ad, eA, afR, dV, aT, cU, h, afU, K, bU, afx, ae, A, aW, Y, afS, cr, r, eZ, fe, g, eE, cm, nC, bz, dr, bN, ck, bF, Jr, z, HK, eo, yA, fD, afT, y, bt, B); #c1(ARHGEF2) c2(XP_005245646) c3(rho guanine nucleotide exchange factor 2 isoform X8) c4(266836) c5( ) c6( ) c7(bM, T, jv, aC, yS, ha, Hn, bS, nA, V, q, ad, eA, afR, dV, aT, cU, h, afU, K, bU, afx, ae, A, aW, Y, afS, cr, r, eZ, fe, g, eE, cm, nC, bz, dr, bN, ck, bF, Jr, z, HK, eo, yA, fD, afT, y, bt, B); #c1(ARHGEF2) c2(XP_005245651) c3(rho guanine nucleotide exchange factor 2 isoform X11) c4( ) c5(266837, 266838, 266839, 266840, 266841, 266842) c6( ) c7(bM, T, jv, aC, yS, ha, Hn, bS, nA, V, q, ad, eA, afR, dV, aT, cU, h, afU, K, bU, afx, ae, A, aW, Y, afS, cr, r, eZ, fe, g, eE, cm, nC, bz, dr, bN, ck, bF, Jr, z, HK, eo, yA, fD, afT, y, bt, B); #c1(ARHGEF2) c2(XP_011508441) c3(rho guanine nucleotide exchange factor 2 isoform X4) c4( ) c5(299158, 299159) c6( ) c7(bM, T, jv, aC, yS, ha, Hn, bS, nA, V, q, ad, eA, afR, dV, aT, cU, h, afU, K, bU, afx, ae, A, aW, Y, afS, cr, r, eZ, fe, g, eE, cm, nC, bz, dr, bN, ck, bF, Jr, z, HK, eo, yA, fD, afT, y, bt, B); #c1(ARHGEF2) c2(XP_011508442) c3(rho guanine nucleotide exchange factor 2 isoform X3) c4(299160) c5(299161, 299162, 299163, 299164, 299165) c6( ) c7(bM, T, jv, aC, yS, ha, Hn, bS, nA, V, q, ad, eA, afR, dV, aT, c, h, afU, K, bU, afx, ae, A, aW, Y, afS, cr, r, eZ, fe, g, eE, cm, nC, bz, dr, bN, ck, bF, Jr, z, HK, eo, yA, fD, afT, y, bt, B); #c1(ARHGEF2) c2(XP_011508444) c3(rho guanine nucleotide exchange factor 2 isoform X13) c4(299166) c5(299167) c6( ) c7(bM, T, jv, aC, yS, ha, Hn, bS, nA, V, q, ad, eA, afR, dV, aT, cu, h, afU, K, bU, afx, ae, A, aW, Y, afS, cr, r, eZ, fe, g, eE, cm, nC, bz, dr, bN, ck, bF, Jr, z, HK, eo, yA, fD, afT, y, bt, B); #c1(ARHGEF2) c2(XP_011508445) c3(rho guanine nucleotide exchange factor 2 isoform X14) c4( ) c5(299168, 299169) c6( ) c7(bM, T, jv, aC, yS, ha, Hn, bS, nA, V, q, ad, eA, afR, dV, aT, cU, h, afU, K, bU, afx, ae, A, aW, Y, afS, cr, r, eZ, fe, g, BE, cm, nC, bz, dr, bN, ck, bF, Jr, z, HK, eo, yA, fD, afT, y, bt, B); #c1(ARHGEF25) c2(NP_001104740) c3(rho guanine nucleotide exchange factor 25 isoform 3) c4(42220, 42221) c5(42222, 42223, 42224, 42225, 42226, 42227) c6( ) c7(bv, aW, cz, aT, afV, By); #c1(ARHGEF25) c2(NP_891992) c3(rho guanine nucleotide exchange factor 25 isoform 1) c4(257430) c5(257431, 257432, 257433, 257434, 257435, 257436, 257437, 257438, 257439, 257440, 257441, 257442) c6( ) c7(bv, aW, cz, aT, afV, By); #c1(ARHGEF26) c2(NP_001238892) c3(rho guanine nucleotide exchange factor 26 isoform 2) c4(88002, 88003) c5(88004, 88005, 88006, 88007, 88008, 88009, 88010, 88011, 88012, 88013, 88014, 88015) c6( ) c7(dD, d, ea, eq, ae, eA, ad); #c1(ARHGEF26) c2(NP_056410) c3(rho guanine nucleotide exchange factor 26 isoform 1) c4(187450, 187451) c5(187452, 187453, 187454, 187455, 187456, 187457, 187458, 187459, 187460, 187461) c6( ) c7(dD, d, ea, eq, ae, eA, ad); #c1(ARHGEF28) c2(NP_001073948) c3(rho guanine nucleotide exchange factor 28 isoform 1) c4(36667, 36668, 36669) c5(36670, 36671, 36672, 36673, 36674, 36675, 36676, 36677, 36678, 36679, 36680, 36681, 36682, 36683, 36684, 36685, 36686, 36687, 36688, 36689, 36690, 36691, 36692) c6( ) c7(oO, bM, AW, dD, iV, c, ky, Ol, ea, oe, aw, ff, eZ, cz, cd, eq, ji, en, PJ, bz, B); #c1(ARHGEF28) c2(NP_001171164) c3(rho guanine nucleotide exchange factor 28 isoform 2) c4(69962, 69963) c5(69964, 69965, 69966, 69967, 69968, 69969, 69970, 69971, 69972, 69973, 69974) c6( ) c7(oO, bM, AW, dD, iV, c, ky, Ol, ea, oe, aw, ff, eZ, cz, cd, eq, ji, en, PJ, bz, B); #c1(ARHGEF3) c2(NP_001122087) c3(rho guanine nucleotide exchange factor 3 isoform 1) c4(46420) c5(46421, 46422, 46423, 46424, 46425, 46426) c6( ) c7(yE, y, fq, bA, afW); #c1(ARHGEF3) c2(NP_001122088) c3(rho guanine nucleotide exchange factor 3 isoform 2) c4(46427) c5(46428, 46429, 46430, 46431, 46432, 46433) c6( ) c7(yE, y, fq, bA, afW); #c1(ARHGEF3) c2(NP_062455) c3(rho guanine nucleotide exchange factor 3 isoform 3) c4(201795) c5(201796, 201797, 201798, 201799, 201800, 201801) c6( ) c7(yE, y, fq, bA, afW); #c1(ARHGEF38) c2(NP_001229658) c3(rho guanine nucleotide exchange factor 38 isoform 1) c4(84706, 84707) c5(84708, 84709, 84710, 84711, 84712, 84713) c6( ) c7(at, cz); #c1(ARHGEF38) c2(NP_060170) c3(rho guanine nucleotide exchange factor 38 isoform 2) c4( ) c5(194613, 194614, 194615, 194616, 194617, 194618) c6( ) c7(at, cz); #c1(ARHGEF4) c2(NP_056135) c3(rho guanine nucleotide exchange factor 4 isoform a) c4(185870) c5(185871, 185872, 185873, 185874, 185875, 185876, 185877, 185878, 185879, 185880, 185881, 185882, 185883, 185884, 185885, 185886, 185887) c6( ) c7(T, s); #c1(ARHGEF4) c2(NP_127462) c3(rho guanine nucleotide exchange factor 4 isoform b) c4(226495) c5(226496, 226497, 226498, 226499, 226500, 226501, 226502, 226503, 226504, 226505, 226506, 226507, 226508) c6( ) c7(T, s); #c1(ARHGEF4) c2(XP_005263738) c3(rho guanine nucleotide exchange factor 4 isoform X1) c4(278517, 278518) c5( ) c6( ) c7(T, s); #c1(ARHGEF4) c2(XP_005263739) c3(rho guanine nucleotide exchange factor 4 isoform X4) c4(278519) c5( ) c6( ) c7(T, s); #c1(ARHGEF4) c2(XP_011509576) c3(rho guanine nucleotide exchange factor 4 isoform X2) c4( ) c5(300150, 300151) c6( ) c7(T, s); #c1(ARHGEF4) c2(XP_011509577) c3(rho guanine nucleotide exchange factor 4 isoform X3) c4( ) c5(300152, 300153) c6( ) c7(T, s); #c1(ARHGEF5) c2(NP_005426) c3(rho guanine nucleotide exchange factor 5) c4(156009, 156010, 156011, 156012) c5(156013, 156014, 156015, 156016, 156017, 156018, 156019, 156020, 156021, 156022, 156023, 156024, 156025, 156026, 156027, 156028, 156029, 156030, 156031, 156032, 156033, 156034, 156035, 156036, 156037, 156038, 156039) c6( ) c7(bM, V, ai, eA, ad, tT, aT, h, nJ, ul, iU, hl, eZ, lo, eE, f, cT, z, U, EY, fD, bC, hE, B, y, afX); #c1(ARHGEF6) c2(NP_004831) c3(rho guanine nucleotide exchange factor 6) c4(150132, 150133, 150134) c5(150135, 150136, 150137, 150138, 150139, 150140, 150141, 150142, 150143) c6( ) c7(nf, lC, eA, afY, nA); #c1(ARHGEF6) c2(XP_011529717) c3(rho guanine nucleotide exchange factor 6 isoform X4) c4(325348) c5(325349) c6( ) c7(nf, lC, eA, afY, nA); #c1(ARHGEF6) c2(XP_011529718) c3(rho guanine nucleotide exchange factor 6 isoform X6) c4( ) c5(325350, 325351, 325352, 325353, 325354) c6( ) c7(nf, lC, eA, afY, nA); #c1(ARHGEF7) c2(NP_001106983) c3(rho guanine nucleotide exchange factor 7 isoform c) c4(42738, 42739, 42740) c5(42741, 42742, 42743, 42744, 42745, 42746) c6( ) c7(T, pf, abM, fd, bB, ci, aE, K, zy, cp, eG, aW, Ls, az, g, as, cm, eM, U, cj, m, Uw, fy, aw, Kk, fr, nA, eW, afR, cU, h, By, he, MD, gi, agb, aq, aga, kT, cA, dB, w, at, B, rf, fZ, aA, ti, j, eA, oT, aT, cy, bK, ae, Y, tu, t, nC, bN, ac, agc, afZ, agf, fD, UF, bt, aZ, ha, ah, ad, agd, nd, xx, d, Gv, nf, gl, tv, aF, nk, iR, iV, ek, LB, age, eU, bE, dO, ei, y, xg); #c1(ARHGEF7) c2(NP_001106984) c3(rho guanine nucleotide exchange factor 7 isoform d) c4(42747, 42748) c5(42749, 42750, 42751, 42752, 42753, 42754, 42755) c6( ) c7(T, pf, abM, fd, bB, ci, aE, K, zy, cp, eG, aW, Ls, az, g, as, cm, eM, U, cj, m, Uw, fy, aw, Kk, fr, nA, eW, afR, cU, h, By, he, MD, gi, agb, aq, aga, KT, cA, dB, w, at, B, rf, fZ, aA, ti, j, eA, oT, aT, cy, bK, ae, Y, tu, t, nC, bN, ac, agc, afZ, agf, fD, UF, bt, aZ, ha, ah, ad, agd, nd, xX, d, Gv, nf, gl, tv, aF, nk, iR, iV, ek, LB, age, eU, bE, dO, ei, y, xg); #c1(ARHGEF7) c2(NP_001106985) c3(rho guanine nucleotide exchange factor 7 isoform a) c4( ) c5(42756, 42757, 42758, 42759, 42760, 42761, 42762, 42763) c6( ) c7(T, pf, abM, fd, bB, ci, aE, K, zy, cp, eG, aW, Ls, az, g, as, cm, eM, U, cj, m, Uw, fy, aw, Kk, fr, nA, eW, afR, cU, h, By, he, MD, gi, agb, aq, aga, kT, cA, dB, w, at, B, rf, fZ, aA, ti, j, eA, oT, aT, cy, bK, ae, Y, tu, t, nC, bN, ac, agc, afZ, agf, fD, UF, bt, aZ, ha, ah, ad, agd, nd, xX, d, Gv, nf, gl, tv, aF, nk, iR, iV, ek, LB, age, eU, bE, d, ei, y, xg); #c1(ARHGEF7) c2(NP_663788) c3(rho guanine nucleotide exchange factor 7 isoform b) c4(241613) c5(241614, 241615, 241616, 241617, 241618, 241619, 241620, 241621, 241622, 241623, 241624, 241625, 241626, 241627, 241628, 241629, 241630, 241631, 241632, 241633, 241634, 241635, 241636, 241637) c6( ) c7(T, pf, abM, fd, bB, ci, aE, K, zy, cp, eG, aW, Ls, az, g, as, cm, eM, U, cj, m, Uw, fy, aw, Kk, fr, nA, eW, afR, cU, h, By, he, MD, gi, agb, aq, aga, KT, cA, dB, w, at, B, rf, fZ, aA, ti, j, eA, oT, aT, cy, bK, ae, Y, tu, t, nC, bN, ac, agc, afZ, agf, fD, UF, bt, aZ, ha, ah, ad, agd, nd, xX, d, Gv, nf, gl, tv, aF, nk, iR, iV, ek, LB, age, eU, bE, dO, ei, y, xg); #c1(ARHGEF7) c2(XP_005254142) c3(rho guanine nucleotide exchange factor 7 isoform X2) c4( ) c5(273010) c6( ) c7(T, pf, abM, fd, bB, ci, aE, K, zy, cp, eG, aW, Ls, az, g, as, cm, eM, U, cj, m, Uw, fy, aw, Kk, fr, nA, eW, afR, cU, h, By, he, MD, gi, agb, aq, aga, KT, cA, dB, w, at, B, rf, fZ, aA, ti, j, eA, oT, aT, cy, bK, ae, Y, tu, t, nC, bN, ac, agc, afZ, agf, fD, UF, bt, aZ, ha, ah, ad, agd, nd, xX, d, Gv, nf, gl, tv, aF, nk, iR, iV, ek, LB, age, eU, bE, dO, ei, y, xg); #c1(ARHGEF7) c2(XP_005254143) c3(rho guanine nucleotide exchange factor 7 isoform X3) c4(273011, 273012) c5(273013) c6( ) c7(T, pf, abM, fd, bB, ci, aE, K, zy, cp, eG, aW, Ls, az, g, as, cm, eM, U, cj, m, Uw, fy, aw, Kk, fr, nA, eW, afR, cU, h, By, he, MD, gi, agb, aq, aga, kT, cA, dB, w, at, B, rf, fZ, aA, ti, j, eA, oT, aT, cy, bK, ae, Y, tu, t, nC, bN, ac, agc, afZ, agf, fD, UF, bt, aZ, ha, ah, ad, agd, nd, xX, d, Gv, nf, gl, tv, af, nk, iR, iV, ek, LB, age, eU, bE, do, ei, y, xg); #c1(ARHGEF7) c2(XP_005254144) c3(rho guanine nucleotide exchange factor 7 isoform X7) c4( ) c5(273014) c6( ) c7(T, pf, abM, fd, bB, ci, aE, K, zy, cp, eG, aW, Ls, az, g, as, cm, eM, U, cj, m, Uw, fy, aw, Kk, fr, nA, eW, afR, cU, h, By, he, MD, gi, agb, aq, aga, kT, cA, dB, w, at, B, rf, fZ, aA, ti, j, eA, oT, aT, cy, bK, ae, Y, tu, t, nC, bN, ac, agc, afZ, agf, fD, UF, bt, aZ, ha, ah, ad, agd, nd, xX, d, Gv, nf, gl, tv, aF, nk, iR, iV, ek, LB, age, eU, bE, d, ei, y, xg); #c1(ARHGEF7) c2(XP_005254146) c3(rho guanine nucleotide exchange factor 7 isoform X9) c4(273015) c5( ) c6( ) c7(T, pf, abM, fd, bB, ci, aE, K, zy, cp, eG, aW, Ls, az, g, as, cm, eM, U, cj, m, Uw, fy, aw, Kk, fr, nA, eW, afR, cU, h, By, he, MD, gi, agb, aq, aga, KT, cA, dB, w, at, B, rf, fZ, aA, ti, j, eA, oT, aT, cy, bK, ae, Y, tu, t, nC, bN, ac, agc, afZ, agf, fD, UF, bt, aZ, ha, ah, ad, agd, nd, xX, d, Gv, nf, gl, tv, aF, nk, iR, lV, ek, LB, age, eU, bE, do, ei, y, xg); #c1(ARHGEF7) c2(XP_005254147) c3(rho guanine nucleotide exchange factor 7 isoform X10) c4(273016) c5(273017, 273018, 273019, 273020, 273021, 273022) c6( ) c7(T, pf, abM, fd, bB, ci, aE, K, zy, cp, eG, aW, Ls, az g, as, cm, eM, U, cj, m, Uw, fy, aw, Kk, fr, nA, eW, afR, cU, h, By, he, MD, gi, agb, aq, aga, kT, cA, dB, w, at, B, rf, fZ, aA, ti, j, eA, oT, aT, cy, bK, ae, Y, tu, t, nC, bN, ac, agc, afZ, agf, fD, UF, bt, aZ, ha, ah, ad, agd, nd, xX, d, Gv, nf, gl, tv, af, nk, iR, iV, ek, LB, age, eU, bE, dO, ei, y, xg); #c1 (ARHGEF7) c2(XP_005254148) c3(rho guanine nucleotide exchange factor 7 isoform X11) c4(273023, 273024) c5(273025, 273026, 273027, 273028, 273029, 273030, 273031) c6( ) c7(T, pf, abM, fd, bB, ci, aE, K, zy, cp, eG, aW, Ls, az, g, as, cm, eM, U, cj, m, Uw, fy, aw, Kk, fr, nA, eW, afR, cU, h, By, he, MD, gi, agb, aq, aga, kT, cA, dB, w, at, B, rf, fZ, aA, ti, j, eA, oT, aT, cy, bK, ae, Y, tu, t, nC, bN, ac, agc, afZ, agf, fD, UF, bt, aZ, ha, ah, ad, agd, nd, xX, d, Gv, nf, gl, tv, aF, nk, iR, iV, ek, LB, age, eU, bE, do, ei, y, xg); #c1(ARHGEF7) c2(XP_011519433) c3(rho guanine nucleotide exchange factor 7 isoform X5) c4( ) c5(314090, 314091, 314092, 314093, 314094) c6( ) c7(T, pf, abM, fd, bB, ci, aE, K, zy, cp, eG, aW, Ls, az, g, as, cm, eM, U, cj, m, Uw, fy, aw, Kk, fr, nA, eW, afR, cU, h, By, he, MD, gi, agb, aq, aga, kT, cA, dB, w, at, B, rf, fZ, aA, ti, j, eA, oT, aT, cy, bK, ae, Y, tu, t, nC, bN, ac, agc, afZ, agf, fD, UF, bt, aZ, ha, ah, ad, agd, nd, xX, d, Gv, nf, gl, tv, aF, nk, iR, iV, ek, LB, age, eU, bE, dO, ei, y, xg); #c1(ARHGEF7) c2(XP_011519435) c3(rho guanine nucleotide exchange factor 7 isoform X14) c4(314095, 314096) c5(314097, 314098, 314099, 314100, 314101, 314102, 314103, 314104, 314105, 314106, 314107, 314108, 314109, 314110, 314111, 314112, 314113, 314114, 314115, 314116, 314117, 314118) c6( ) c7(T, pf, abM, fd, bB, ci, aE, K, zy, cp, eG, aW, Ls, az, g, as, cm, eM, U, cj, m, Uw, fy, aw, Kk, fr, nA, eW, afR, cU, h, By, he, MD, gi, agb, aq, aga, kT, cA, dB, w, at, B, rf, fZ, aA, ti, j, eA, oT, aT, cy, bK, ae, Y, tu, t, nC, bN, ac, agc, afZ, agf, fD, UF, bt, aZ, ha, ah, ad, agd, nd, xX, d, Gv, nf, gl, tv, aF, nk, iR, iV, ek, LB, age, eU, bE, dO, ei, y, xg); #c1(ARHGEF9) c2(NP_001166950) c3(rho guanine nucleotide exchange factor 9 isoform 2) c4(68712) c5(68713, 68714, 68715, 68716, 68717, 68718) c6( ) c7(Il, lF, ha, agg, nf, nA, Ku, B); #c1(ARHGEF9) c2(NP_001166951) c3(rho guanine nucleotide exchange factor 9 isoform 3) c4(68719) c5(68720, 68721, 68722, 68723, 68724) c6( ) c7(Il, lF, ha, agg, nf, nA, Ku, B); #c1(ARHGEF9) c2(NP_056000) c3(rho guanine nucleotide exchange factor 9 isoform 1) c4(185054, 185055) c5(185056, 185057, 185058, 185059, 185060, 185061, 185062, 185063, 185064, 185065, 185066, 185067) c6( ) c7(U, lF, ha, agg, nf, nA, Ku, B); #c1(ARHGEF9) c2(XP_005262306) c3(rho guanine nucleotide exchange factor 9 isoform X1) c4( ) c5(277821, 277822, 277823, 277824, 277825, 277826) c6( ) c7(Il, lF, ha, agg, nf, nA, Ku, B); #c1(ARHGEF9) c2(XP_011529192) c3(rho guanine nucleotide exchange factor 9 isoform X2) c4(324593) c5( ) c6( ) c7(Il, lF, ha, agg, nf, nA, Ku, B); #c1(ARHGEF9) c2(XP_011529193) c3(rho guanine nucleotide exchange factor 9 isoform X4) c4(324594) c5(324595, 324596, 324597, 324598, 324599, 324600) c6( ) c7(Il, lF, ha, agg, nf, nA, Ku, B); #c1(ARHGEF9) c2(XP_011529194) c3(rho guanine nucleotide exchange factor 9 isoform X5) c4( ) c5(324601) c6( ) c7(Il, lF, ha, agg, nf, nA, Ku, B); #c1(ARID1B) c2(NP_059989) c3(AT-rich interactive domain-containing protein 18 isoform 1) c4(193510, 193511) c5(193512, 193513, 193514, 193515, 193516, 193517, 193518, 193519, 193520, 193521, 193522, 193523, 193524) c6( ) c7(T, aE, agh, agi, cz, cm, cy, agj, nA, mv, WI, agk, B, zV, bN); #c1(ARID1B) c2(NP_065783) c3(AT-rich interactive domain-containing protein (B isoform 2) c4(205420) c5(205421, 205422, 205423, 205424, 205425, 205426, 205427) c6( ) c7(T, aE, agh, agi, cz, cm, cy, agj, nA, mv, Wl, agk, B, zV, bN); #c1(ARID1B) c2(XP_005267126) c3(AT-rich interactive domain-containing protein 1B isoform X1) c4(280769, 280770, 280771) c5(280772, 280773, 280774, 280775, 280776, 280777, 280778, 280779, 280780, 280781, 280782, 280783, 280784, 280785, 280786, 280787, 280788, 280789, 280790) c6( ) c7(T, aE, agh, agi, cz, cm, cy, agj, nA, mv, Wl, agk, B, zV, bN); #c1(ARID1B) c2(XP_011534288) c3(AT-rich interactive domain-containing protein IB isoform X4) c4(330660) c5(330661, 330662, 330663, 330664, 330665, 330666, 330667, 330668, 330669, 330670) c6( ) c7(T, aE, agh, agi, cz, cm, cy, agj, nA, mv, Wl, agk, B, zV, bN); #c1(ARID2) c2(NP_689854) c3(AT-rich interactive domain-containing protein 2) c4(244164, 244165) c5(244166, 244167, 244168, 244169, 244170, 244171, 244172, 244173, 244174, 244175, 244176, 244177, 244178, 244179, 244180, 244181, 244182, 244183, 244184, 244185, 244186, 244187) c6( ) c7(eM, Y, jx, afi, aq, afv, iR, B); #c1(ARID2) c2(XP_011536327) c3(AT-rich interactive domain-containing protein 2 isoform X2) c4(333672) c5( ) c6( ) c7(eM, Y, jx, afi, aq, afv, iR, B); #c1(ARID3A) c2(NP_005215) c3(AT-rich interactive domain-containing protein 3A) c4(154089) c5(154090, 154091, 154092, 154093, 154094, 154095, 154096) c6( ) c7(LB, ck, A, fG, cz, MC); #c1(ARID3B) c2(NP_006456) c3(AT-rich interactive domain-containing protein 3B) c4(165361) c5(165362, 165363, 165364, 165365, 165366, 165367, 165368, 165369, 165370, 165371, 165372, 165373, 165374, 165375, 165376, 165377, 165378, 165379, 165380) c6( ) c7(cm, t, m, bP); #c1(ARID4A) c2(NP_002883) c3(AT-rich interactive domain-containing protein 4A isoform 1) c4(133173) c5(133174, 133175, 133176, 133177, 133178, 133179) c6( ) c7(ck, Mb, bP, aW, agl, r, aT, K, eA, bt, q); #c1(ARID4A) c2(NP_075376) c3(AT-rich interactive domain-containing protein 4A isoform II) c4(213774) c5(213775, 213776, 213777, 213778, 213779, 213780) c6( ) c7(ck, Mb, bP, aW, agl, r, aT, K, eA, bt, q); #c1(ARID4A) c2(NP_075377) c3(AT-rich interactive domain-containing protein 4A isoform Ill) c4(213781) c5(213782, 213783, 213784, 213785, 213786, 213787, 213788, 213789, 213790, 213791, 213792, 213793, 213794, 213795, 213796, 213797) c6( ) c7(ck, Mb, bP, aW, agl, r, aT, K, eA, bt, q); #c1(ARID4A) c2(XP_011535356) c3(AT-rich interactive domain-containing protein 4A isoform X1) c4(332409) c5( ) c6( ) c7(ck, Mb, bP, aW, agl, r, aT, K, eA, bt, q); #c1(ARID4B) c2(NP_057458) c3(AT-rich interactive domain-containing protein 48 isoform 1) c4(191011, 191012, 191013) c5(191014, 191015, 191016, 191017, 191018, 191019, 191020, 191021, 191022, 191023, 191024, 191025, 191026, 191027, 191028, 191029, 191030, 191031, 191032, 191033, 191034, 191035) c6( ) c7(T, B, d, c, bP, aW, aZ, qK, aT, aq, v, agm, m, eA, iR, t, rL); #c1(ARID4B) c2(NP_112739) c3(AT-rich interactive domain-containing protein 48 isoform 2) c4(221599, 221600) c5(221601, 221602, 221603, 221604, 221605, 221606) c6( ) c7(T, B, d, c, bP, aW, aZ, qK, aT, aq, v, agm, m, eA, iR, t, rL); #c1(ARID4B) c2(XP_005273217) c3 (AT-rich interactive domain-containing protein 48 isoform X3) c4( ) c5(284364, 284365, 284366, 284367, 284368, 284369) c6( ) c7(T, B, d, c, bP, aW, aZ, qK, aT, aq, v, agm, m, eA, iR, t, rL); #c1(ARID4B) c2(XP_011542515) c3(AT-rich interactive domain-containing protein 4B isoform X4) c4( ) c5(341059) c6( ) c7(T, B, d, c, bP, aW, aZ, qK, aT, aq, v, agm, m, eA, iR, t, rL); #c1(ARID5B) c2(NP_001231567) c3(AT-rich interactive domain-containing protein 5B isoform 2) c4(87299) c5(87300, 87301, 87302, 87303, 87304, 87305, 87306) c6( ) c7(bL, ck, x, dD, ea, WL, Bj, qK, eZ, hD, dq, hn, j, k, eq, y, bt, agn); #c1(ARID5B) c2(NP_115575) c3(AT-rich interactive domain-containing protein 5B isoform 1) c4(223262) c5(223263, 223264, 223265, 223266, 223267, 223268) c6( ) c7(bL, ck, x, dD, ea, WL, Bj, qK, eZ, HD, dq, hn, j, k, eq, y, bt, agn); #c1(ARIH1) c2(NP_005735) c3(E3 ubiquitin-protein ligase ARIH1) c4(158953, 158954) c5(158955, 158956, 158957, 158958, 158959, 158960, 158961, 158962, 158963, 158964, 158965, 158966) c6( ) c7(cU, pR, ae, ad, an); #c1(ARL1) c2(NP_001168) c3(ADP-ribosylation factor-like protein 1 isoform 1) c4(69489, 69490) c5(69491, 69492, 69493, 69494, 69495, 69496, 69497, 69498, 69499, 69500, 69501, 69502, 69503, 69504, 69505, 69506) c6( ) c7(aq); #c1(ARL1) c2(NP_001287997) c3(ADP-ribosylation factor-like protein 1 isoform 2) c4(113343) c5(113344, 113345, 113346, 113347, 113348, 113349) c6( ) c7(aq); #c1(ARL1I) c2(NP_612459) c3(ADP-ribosylation factor-like protein (1) c4(236564) c5(236565, 236566, 236567, 236568, 236569, 236570) c6( ) c7(GI, T, ae, av, qw, Ub, aW, Y, aT, cj, r, hn, Gn, ci, m, t, rA, bz, q, ad); #c1(ARL13A) c2(NP_001155963) c3(ADP-ribosylation factor-like protein 13A) c4(61648) c5(61649, 61650, 61651, 61652, 61653, 61654) c6( ) c7(ago); #c1(ARL13A) c2(XP_011529252) c3(ADP-ribosylation factor-like protein 13A isoform X1) c4(324700) c5(324701, 324702, 324703, 324704, 324705, 324706) c6( ) c7(ago); #c1(ARL13B) c2(NP_001167621) c3(ADP-ribosylation factor-like protein 13B isoform 1) c4(69428) c5(69429, 69430, 69431, 69432, 69433, 69434, 69435, 69436, 69437, 69438) c6( ) c7(Ng, agp); #c1(ARL13B) c2(NP_001167622) c3(ADP-ribosylation factor-like protein 13B isoform 3) c4( ) c5(69439, 69440, 69441, 69442, 69443) c6( ) c7(Ng, agp); #c1(ARL13B) c2(NP_659433) c3(ADP-ribosylation factor-like protein 13B isoform 2) c4(239860) c5(239861, 239862, 239863, 239864, 239865, 239866, 239867, 239868, 239869, 239870, 239871, 239872) c6( ) c7(Ng, agp); #c1(ARL13B) c2(XP_011510835) c3(ADP-ribosylation factor-like protein 13B isoform X2) c4(301294, 301295) c5(301296) c6( ) c7(Ng, agp); #c1(ARL14) c2(NP_079323) c3(ADP-ribosylation factor-like protein 14) c4(217741) c5(217742, 217743, 217744, 217745, 217746, 217747) c6( ) c7(hb); #c1(ARL14EP) c2(NP_689529) c3(ARL14 effector protein) c4(243001, 243002) c5(243003, 243004, 243005, 243006, 243007, 243008, 243009, 243010, 243011, 243012, 243013, 243014) c6( ) c7(gv, nA); #c1(ARL15) c2(NP_061960) c3(ADP-ribosylation factor-like protein 15) c4(201318) c5(201319, 201320, 201321, 201322, 201323, 201324) c6( ) c7(bL, W, aw, v, j, y); #c1(ARL2) c2(NP_001186674) c3(ADP-ribosylation factor-like protein 2 isoform 2) c4(78308) c5(78309, 78310, 78311, 78312, 78313, 78314) c6( ) c7(T, aE, aW, Y, aT, K, g); #c1(ARL2) c2(NP_001658) c3(ADP-ribosylation factor-like protein 2 isoform 1) c4(119822) c5(119823, 119824, 119825, 119826, 119827, 119828, 119829, 119830, 119831, 119832) c6( ) c7(T, aE, aW, Y, aT, K, g); #c1(ARL2BP) c2(NP_036238) c3(ADP-ribosylation factor-like protein 2-binding protein) c4(172577) c5(172578, 172579, 172580, 172581, 172582, 172583) c6( ) c7(T, aE, eX, h, eV, By, agq, aF, ci, K); #c1(ARL3) c2(NP_004302) c3(ADP-ribosylation factor-like protein 3) c4(144990) c5(144991, 144992, 144993, 144994, 144995, 144996) c6( ) c7(j); #c1(ARL4A) c2(NP_001182325) c3(ADP-ribosylation factor-like protein 4A) c4(74774, 74775) c5(74776, 74777, 74778, 74779, 74780, 74781, 74782, 74783, 74784, 74785, 74786, 74787, 74788, 74789, 74790, 74791, 74792, 74793, 74794, 74795, 74796, 74797, 74798, 74799, 74800, 74801, 74802, 74803) c6( ) c7(eA); #c1(ARL4C) c2(NP_001269360) c3(ADP-ribosylation factor-like protein 4C isoform 1) c4(101282) c5(101283, 101284, 101285, 101286, 101287, 101288) c6( ) c7(nZ, iU, ht, bS, ct, g, Z); #c1(ARL4C) c2(NP_005728) c3(ADP-ribosylation factor-like protein 4C isoform 2) c4(158917) c5(158918, 158919, 158920, 158921, 158922, 158923) c6( ) c7(nZ, iU, ht, bS, ct, g, Z); #c1(ARL4D) c2(NP_001652) c3(ADP-ribosylation factor-like protein 4D) c4(119786) c5(119787, 119788, 119789, 119790, 119791, 119792) c6( ) c7(dB, eA, Tp); #c1(ARL5A) c2(NP_001032251) c3(ADP-ribosylation factor-like protein 5A isoform 2) c4(31175) c5(31176, 31177, 31178, 31179, 31180, 31181, 31182, 31183) c6( ) c7(q, r); #c1(ARL5A) c2(NP_036229) c3(ADP-ribosylation factor-like protein 5A isoform 1) c4(172467) c5(172468, 172469, 172470, 172471, 172472, 172473, 172474, 172475) c6( ) c7(q, r); #c1(ARL5B) c2(NP_848930) c3(ADP-ribosylation factor-like protein 5B) c4(254230) c5(254231, 254232, 254233, 254234, 254235, 254236) c6( ) c7(Y, af); #c1(ARL6) c2(NP_816931) c3(ADP-ribosylation factor-like protein 6) c4(253048, 253049) c5(253050, 253051, 253052, 253053, 253054, 253055, 253056, 253057, 253058, 253059, 253060, 253061, 253062, 253063, 253064, 253065, 253066, 253067) c6( ) c7(ags, gU, lR, agr, v, Tp); #c1(ARL6) c2(XP_011511532) c3(ADP-ribosylation factor-like protein 6 isoform X3) c4( ) c5(302217, 302218, 302219, 302220, 302221, 302222) c6( ) c7(ags, gU, lR, agr, v, Tp); #c1(ARL6IP1) c2(NP_055976) c3 (ADP-ribosylation factor-like protein 6-interacting protein 1) c4(184840, 184841, 184842) c5(184843, 184844, 184845, 184846, 184847, 184848, 184849) c6( ) c7(ea, Y, if, h, agt, qN, bz, ad); #c1(ARL6IP5) c2(NP_006398) c3(PRA1 family protein 3) c4(164704, 164705) c5(164706, 164707, 164708, 164709, 164710, 164711, 164712, 164713, 164714, 164715, 164716, 164717) c6( ) c7(ck, T, Y, ff, aA, r, it, dL, ey, aF, en, el); #c1(ARMC1) c2(NP_001273631) c3(armadillo repeat-containing protein 1 isoform 2) c4(105505) c5(105506, 105507, 105508, 105509, 105510, 105511) c6( ) c7(ey); #c1(ARMC1) c2(NP_060590) c3(armadillo repeat-containing protein 1 isoform 1) c4(196705) c5(196706, 196707, 196708, 196709, 196710, 196711) c6( ) c7(ey); #c1(ARMC10) c2(NP_001154481) c3(armadillo repeat-containing protein 10 isoform b) c4(61045) c5(61046, 61047, 61048, 61049, 61050, 61051) c6( ) c7(aq, B); #c1(ARMC10) c2(NP_001154482) c3(armadillo repeat-containing protein 10 isoform c) c4(61052) c5(61053, 61054, 61055, 61056, 61057, 61058) c6( ) c7(aq, B); #c1(ARMC10) c2(NP_001154483) c3(armadillo repeat-containing protein 10 isoform e) c4(61059) c5(61060, 61061, 61062, 61063, 61064, 61065) c6( ) c7(aq, B); #c1(ARMC10) c2(NP_001154484) c3(armadillo repeat-containing protein 10 isoform d) c4(61066) c5(61067, 61068, 61069, 61070, 61071, 61072) c6( ) c7(aq, B); #c1(ARMC10) c2(NP_001154485) c3(armadillo repeat-containing protein 10 isoform f) c4(61073) c5(61074, 61075, 61076, 61077, 61078, 61079) c6( ) c7(aq, B); #c1(ARMC10) c2(NP_114/11) c3(armadillo repeat-containing protein 10 isoform a) c4(222183) c5(222184, 222185, 222186, 222187, 222188, 222189) c6( ) c7(aq, B); #c1(ARMC2) c2(NP_115507) c3(armadillo repeat-containing protein 2 isoform a) c4(222969, 222970) c5(222971, 222972, 222973, 222974, 222975, 222976, 222977, 222978, 222979, 222980, 222981, 222982) c6( ) c7(aj, ad); #c1(ARMC2) c2(XP_011534471) c3(armadillo repeat-containing protein 2 isoform X4) c4(330943) c5( ) c6( ) c7(aj, ad); #c1(ARMC2) c2(XP_011534478) c3(armadillo repeat-containing protein 2 isoform X10) c4( ) c5(330944, 330945, 330946, 330947, 330948, 330949) c6( ) c7(aj, ad); #c1(ARMC3) c2(NP_001269674) c3(armadillo repeat-containing protein 3 isoform 2) c4(102495) c5(102496, 102497, 102498, 102499, 102500, 102501) c6( ) c7(cl, T, aE, hL, bu); #c1(ARMC3) c2(NP_001269675) c3(armadillo repeat-containing protein 3 isoform 3) c4(102502) c5(102503, 102504, 102505, 102506, 102507, 102508) c6( ) c7(cl, T, aE, hL, bu); #c1(ARMC3) c2(NP_001269676) c3(armadillo repeat-containing protein 3 isoform 4) c4(102509) c5( ) c6( ) c7(cl, T, aE, hL, bu); #c1(ARMC3) c2(NP_775104) c3(armadillo repeat-containing protein 3 isoform 1) c4(249515) c5(249516, 249517, 249518, 249519, 249520, 249521) c6( ) c7(cl, T, aE, hL, bu); #c1(ARMC3) c2(XP_005252438) c3(armadillo repeat-containing protein 3 isoform X2) c4( ) c5(271807, 271808, 271809, 271810, 271811, 271812) c6( ) c7(cl, T, aE, hL, bu); #c1(ARMC4) c2(NP_001276950) c3(armadillo repeat-containing protein 4 isoform 2) c4(108696) c5(108697, 108698, 108699, 108700, 108701, 108702) c6( ) c7(agu, BV, Pf, MG); #c1(ARMC4) c2(NP_060546) c3(armadillo repeat-containing protein 4 isoform 1) c4(196499, 196500, 196501) c5(196502, 196503, 196504, 196505, 196506, 196507) c6( ) c7(agu, BV, Pf, MG); #c1(ARMC4) c2(XP_011517831) c3(armadillo repeat-containing protein 4 isoform X1) c4(31/760) c5(311761) c6( ) c7(agu, BV, Pf, MG); #c1(ARMC4) c2(XP_011517832) c3(armadillo repeat-containing protein 4 isoform X2) c4( ) c5(311762, 311763, 311764, 311765, 311766, 311767) c6( ) c7(agu, BV, Pf, MG); #c1(ARMC4) c2(XP_011517838) c3(armadillo repeat-containing protein 4 isoform X8) c4( ) c5(31/768, 311769, 311770, 311771, 311772, 311773, 311774, 311775, 311776, 311777, 311778, 311779, 311780, 311781, 311782, 311783) c6( ) c7(agu, BV, Pf, MG); #c1(ARMC5) c2(NP_001098717) c3(armadillo repeat-containing protein 5 isoform a precursor) c4( ) c5(41538, 41539, 41540, 41541, 41542, 41543) c6( ) c7(Ja, agv); #c1(ARMC5) c2(NP_001275696) c3(armadillo repeat-containing protein 5 isoform c) c4(107095, 107096, 107097) c5(107098, 107099, 107100, 107101, 107102, 107103, 107104, 107105, 107106, 107107, 107108, 107109, 107110, 107/11, 107112, 107113, 107114, 107115) c6( ) c7(Ja, agv); #c1(ARMC5) c2(NP_001288749) c3(armadillo repeat-containing protein 5 isoform d) c4(113967) c5( ) c6( ) c7(Ja, agv); #c1(ARMC5) c2(NP_079018) c3(armadillo repeat-containing protein 5 isoform b precursor) c4(216594) c5(216595, 216596, 216597, 216598, 216599, 216600, 216601) c6( ) c7(Ja, agv); #c1(ARMC8) c2(NP_001253970) c3(armadillo repeat-containing protein 8 isoform 3) c4(94321) c5(94322, 94323, 94324, 94325, 94326, 94327) c6( ) c7(T, bz, eM); #c1(ARMC8) c2(NP_001253971) c3(armadillo repeat-containing protein 8 isoform 4) c4(94328) c5(94329, 94330, 94331, 94332, 94333, 94334) c6( ) c7(T, bz, eM); #c1(ARMCB) c2(NP_054873) c3(armadillo repeat-containing protein 8 isoform 1) c4(178144) c5(178145, 178146, 178147, 178148, 178149, 178150, 178151, 178152, 178153, 178154, 178155, 178156, 178157, 178158, 178159, 178160, 178161) c6( ) c7(T, bz, eM); #c1(ARMC8) c2(NP_056211) c3(armadillo repeat-containing protein 8 isoform 2) c4(186380, 186381, 186382) c5(186383, 186384, 186385, 186386, 186387, 186388, 186389, 186390, 186391, 186392, 186393, 186394, 186395, 186396, 186397, 186398, 186399, 186400, 186401, 186402, 186403, 186404, 186405) c6( ) c7(T, bz, eM); #c1(ARMC8) c2(XP_006713627) c3(armadillo repeat-containing protein 8 isoform X2) c4(287242, 287243) c5(287244, 287245, 287246, 287247, 287248, 287249, 287250, 287251, 287252, 287253, 287254, 287255) c6( ) c7(T, bz, eM); #c1(ARMC8) c2(XP_006713629) c3(armadillo repeat-containing protein 8 isoform X3) c4(287256, 287257) c5( ) c6( ) c7(T, bz, eM); #c1(ARMC8) c2(XP_011510936) c3(armadillo repeat-containing protein 8 isoform X5) c4( ) c5(301408) c6( ) c7(T, bz, eM); #c1(ARMC9) c2(NP_001258395) c3(lisH domain-containing protein ARMC9 isoform 1) c4( ) c5(96045, 96046, 96047, 96048, 96049, 96050, 96051, 96052, 96053, 96054, 96055, 96056) c6( ) c7(li, afR, agx, T, W, lE, Pf, aW, HA, dV, aT, agw, h, fr, agz, agy, hH, iJ, MG); #c1(ARMC9) c2(XP_011510213) c3(lisH domain-containing protein ARMC9 isoform X1) c4(300790) c5( ) c6( ) c7(li, afR, agx, T, W, lE, Pf, aW, HA, dV, aT, agw, h, fr, agz, agy, hH, iJ, MG); #c1(ARMC9) c2(XP_011510216) c3(lisH domain-containing protein ARMC9 isoform X10) c4(300791) c5( ) c6( ) c7(li, afR, agx, T, W, lE, Pf, aW, HA, dV, aT, agw, h, fr, agz, agy, hH, iJ, MG); #c1(ARMC9) c2(XP_011510221) c3(lisH domain-containing protein ARMC9 isoform X15) c4( ) c5(300792) c6( ) c7(li, afR, agx, T, W, lE, Pf, aW, HA, dV, aT, agw, h, fr, agz, agy, hH, iJ, MG); #c1(ARMC9) c2(XP_011510222) c3(lisH domain-containing protein ARMC9 isoform X16) c4(300793) c5(300794, 300795, 300796, 300797, 300798, 300799) c6( ) c7(li, afR, agx, T, W, lE, Pf, aW, HA, dV, aT, agw, h, fr, agz, agy, hH, iJ, MG); #c1(ARMCX1) c2(NP_057692) c3(armadillo repeat-containing X-linked protein 1) c4(192370, 192371) c5(192372, 192373, 192374, 192375, 192376, 192377, 192378, 192379, 192380, 192381, 192382, 192383) c6( ) c7(q); #c1(ARMS2) c2(NP_001093137) c3(age-related maculopathy susceptibility protein 2) c4(39816) c5(39817, 39818, 39819, 39820, 39821, 39822) c6( ) c7(agA, bL, dD, aw, agC, cz, j, eq, cd, ad, agB, agD, acd, db, Um, af, lR, aaP, fe, da, Uk, W, nb, oh, oQ, GF); #c1(ARMT1) c2(NP_001273491) c3(UPF0364 protein C6orf211 isoform b) c4( ) c5(105047, 105048, 105049, 105050, 105051) c6( ) c7(ey, ac); #c1(ARMT1) c2(NP_078849) c3(UPF0364 protein C6orf211 isoform a) c4(215710, 215711) c5(215712, 215713, 215714, 215715, 215716, 215717, 215718) c6( ) c7(ey, ac); #c1(ARNT) c2(XP_011507849) c3(aryl hydrocarbon receptor nuclear translocator isoform X9) c4(297993) c5(297994, 297995, 297996, 297997, 297998, 297999) c6( ) c7(bL, T, al, aA, n, Ns, pz, ad, dP, aT, cy, dL, ji, K, an, ae, yo, aW, Y, aq, aF, Xk, iR, t, op, ov, vA, bo, mg, cj, at, mf, m, k, bt, B); #c1(ARNT2) c2(NP_055677) c3(aryl hydrocarbon receptor nuclear translocator 2) c4(182686, 182687, 182688, 182689) c5(182690, 182691, 182692, 182693, 182694, 182695, 182696, 182697, 182698, 182699, 182700, 182701, 182702, 182703, 182704, 182705, 182706, 182707, 182708, 182709, 182710, 182711, 182712, 182713, 182714, 182715, 182716, 182717, 182718) c6( ) c7(T, aW, aw, agE, EQ, aT, cm, Pp, bK, v, rz, cd, bN); #c1(ARNTL) c2(NP_001025443) c3(aryl hydrocarbon receptor nuclear translocator-like protein 1 isoform a) c4(29535, 29536) c5(29537, 29538, 29539, 29540, 29541, 29542, 29543, 29544, 29545, 29546, 29547, 29548, 29549, 29550, 29551, 29552, 29553, 29554, 29555, 29556, 29557, 29558, 29559, 29560, 29561, 29562, 29563, 29564, 29565, 29566, 29567, 29568, 29569, 29570, 29571, 29572, 29573, 29574, 29575, 29576, 29577, 29578) c6( ) c7(bL, T, gU, Nt, cz, aj, bS, j, ct, Ct, ci, hy, v, q, J, cd, ad, eA, eW, MR, Os, aT, dl, bk, oi, ae, eL, lr, xQ, d, BN, B, nO, iU, aW, Y, r, agF, Z, cm, bz, ck, qJ, nM, iT, mg, ei, ne); #c1(ARNTL) c2(NP_001025444) c3(aryl hydrocarbon receptor nuclear translocator-like protein 1 isoform b) c4(29579) c5(29580, 29581, 29582, 29583, 29584, 29585, 29586, 29587, 29588, 29589, 29590, 29591, 29592, 29593, 29594, 29595, 29596, 29597, 29598, 29599, 29600, 29601, 29602, 29603) c6( ) c7(bL, T, gU, Nt, cz, aj, bS, j, ct, Ct, ci, hy, v, q, J, cd, ad, eA, eW, MR, Os, aT, dL, bK, oi, ae, eL, lr, xQ, d, BN, B, nO, iU, aW, Y, r, agF, Z, cm, bz, ck, qJ, nM, iT, mg, ei, ne); #c1(ARNTL) c2(NP_001284651) c3(aryl hydrocarbon receptor nuclear translocator-like protein 1 isoform c) c4(112243, 112244, 112245, 112246, 112247) c5(112248, 112249, 112250, 112251, 112252, 112253, 112254, 112255, 112256, 112257, 112258, 112259) c6( ) c7(bL, T, gU, Nt, cz, aj, bS, j, ct, Ct, ci, hy, v, q, J, cd, ad, eA, eW, MR, Os, aT, dL, bk, oi, ae, eL, lr, xQ, d, BN, B, nO, iU, aW, Y, r, agF, Z, cm, bz, ck, qJ, nM, iT, mg, ei, ne); #c1(ARNTL) c2(NP_001284653) c3 (aryl hydrocarbon receptor nuclear translocator-like protein 1 isoform d) c4(112260, 112261) c5(112262, 112263, 112264, 112265, 112266, 112267, 112268, 112269, 112270, 112271, 112272, 112273) c6( ) c7(bL, T, gU, Nt, cz, aj, bS, j, ct, Ct, ci, hy, v, q, J, cd, ad, eA, eW, MR, Os, aT, dL, bK, oi, ae, eL, lr, xQ, d, BN, B, nO, iU, aW, Y, r, agF, Z, cm, bz, ck, qJ, nM, iT, mg, ei, ne); #c1(ARNTL) c2(XP_011518407) c3(aryl hydrocarbon receptor nuclear translocator-like protein 1 isoform X5) c4(312386) c5( ) c6( ) c7(bL, T, gU, Nt, cz, aj, bS, j, ct, Ct, ci, hy, v, q, J, cd, ad, eA, eW, MR, Os, aT, dL, bK, oi, ae, eL, lr, xQ, d, BN, B, nO, iU, aW, Y, r, agF, Z, cm, bz, ck, qJ, nM, iT, mg, ei, ne); #c1(ARNTL) c2(XP_011518411) c3(aryl hydrocarbon receptor nuclear translocator-like protein 1 isoform X6) c4(312387) c5(312388, 312389, 312390, 312391, 312392) c6( ) c7(bL, T, gU, Nt, cz, aj, bS, j, ct, Ct, ci, hy, v, q, J, cd, ad, eA, eW, MR, Os, aT, dL, bK, oi, ae, eL, lr, xQ, d, BN, B, nO, iU, aW, Y, r, agF, Z, cm, bz, ck, qJ, nM, iT, mg, ei, ne); #c1(ARNTL) c2(XP_011518413) c3 (aryl hydrocarbon receptor nuclear translocator-like protein 1 isoform X7) c4(312393, 312394, 312395) c5( ) c6( ) c7(bL, T, gU, Nt, cz, aj, bS, j, ct, Ct, ci, hy, v, q, J, cd, ad, eA, eW, MR, Os, aT, dL, bK, oi, ae, eL, lr, xQ, d, BN, B, nO, iU, aW, Y, r, agF, Z, cm, bz, ck, qJ, nM, iT, mg, ei, ne); #c1(ARNTL2) c2(NP_001234931) c3(aryl hydrocarbon receptor nuclear translocator-like protein 2 isoform 2) c4(87832) c5(87833, 87834, 87835, 87836, 87837, 87838) c6( ) c7(bE, aW, QU, Gs, r, aT, Gd, bB, agG, bK, ey, ei, Z, q); #c1(ARNTL2) c2(NP_001234932) c3(aryl hydrocarbon receptor nuclear translocator-like protein 2 isoform 3) c4(87839, 87840) c5( ) c6( ) c7(bE, aW, QU, Gs, r, aT, Gd, bB, agG, bK, ey, ei, Z, q); #c1(ARNTL2) c2(NP_001234933) c3(aryl hydrocarbon receptor nuclear translocator-like protein 2 isoform 4) c4(87841) c5(87842, 87843, 87844, 87845, 87846, 87847, 87848, 87849, 87850, 87851, 87852, 87853, 87854, 87855, 87856, 87857, 87858, 87859) c6( ) c7(bE, aW, QU, Gs, r, aT, Gd, bB, agG, bK, ey, ei, Z, q); #c1(ARNTL2) c2(NP_064568) c3 (aryl hydrocarbon receptor nuclear translocator-like protein 2 isoform 1) c4(203019) c5(203020, 203021, 203022, 203023, 203024, 203025, 203026) c6( ) c7(bE, aW, QU, Gs, r, aT, Gd, bB, agG, bK, ey, ei, Z, q); #c1(ARNTL2) c2(XP_011519071) c3(aryl hydrocarbon receptor nuclear translocator-like protein 2 isoform X5) c4(313665) c5(313666, 313667, 313668, 313669, 313670, 313671) c6( ) c7(bE, aW, QU, Gs, r, aT, Gd, bB, agG, bK, ey, ei, Z, q); #c1(ARPC1A) c2(NP_001177925) c3(actin-related protein 2/3 complex subunit IA isoform 2) c4(73180) c5(73181, 73182, 73183, 73184, 73185, 73186) c6( ) c7(aC, aE); #c1(ARPC1A) c2(NP_006400) c3(actin-related protein 2/3 complex subunit IA isoform 1) c4(164739) c5(164740, 164741, 164742, 164743, 164744, 164745) c6( ) c7(aC, aE); #c1(ARPC1B) c2(NP_005711) c3(actin-related protein 2/3 complex subunit (B) c4(158703, 158704, 158705, 158706, 158707, 158708, 158709, 158710) c5(158711, 158712, 158713, 158714, 158715, 158716, 158717, 158718, 158719, 158720, 158721, 158722, 158723, 158724, 158725, 158726, 158727, 158728, 158729, 158730, 158731, 158732, 158733, 158734, 158735, 158736, 158737, 158738, 158739, 158740, 158741, 158742, 158743, 158744, 158745, 158746, 158747, 158748, 158749, 158750, 158751, 158752, 158753, 158754, 158755, 158756, 158757, 158758, 158759, 158760, 158761, 158762, 158763, 158764, 158765, 158766, 158767, 158768, 158769) c6( ) c7(Y, aF, aA); #c1(ARPC2) c2(NP_005722) c3(actin-related protein 2/3 complex subunit 2) c4(158829, 158830) c5(158831, 158832, 158833, 158834, 158835, 158836, 158837, 158838, 158839, 158840, 158841, 158842, 158843, 158844, 158845, 158846) c6( ) c7(fe, ha, iV); #c1(ARPC3) c2(NP_001265485) c3(actin-related protein 2/3 complex subunit 3 isoform 1) c4(98690, 98691) c5(98692, 98693, 98694, 98695, 98696, 98697, 98698, 98699, 98700, 98701, 98702, 98703) c6( ) c7(agH); #c1(ARPC5) c2(NP_001257368) c3(actin-related protein 2/3 complex subunit 5 isoform 2) c4(95261) c5(95262, 95263, 95264, 95265, 95266, 95267) c6( ) c7(Qu, bv, T, iK); #c1(ARPC5) c2(NP_005708) c3(actin-related protein 2/3 complex subunit 5 isoform 1) c4(158689, 158690) c5(158691, 158692, 158693, 158694, 158695, 158696, 158697, 158698, 158699, 158700, 158701, 158702) c6( ) c7(Qu, bv, T, iK); #c1(ARPIN) c2(NP_872422) c3(arpin isoform 1) c4( ) c5(256324, 256325, 256326, 256327, 256328, 256329) c6( ) c7(na); #c1(ARPP21) c2(NP_001020240) c3(cAMP-regulated phosphoprotein 21 isoform 2) c4( ) c5(28275, 28276, 28277, 28278, 28279, 28280, 28281, 28282, 28283, 28284, 28285, 28286, 28287, 28288, 28289, 28290, 28291, 28292, 28293, 28294, 28295, 28296, 28297, 28298, 28299, 28300, 28301, 28302, 28303, 28304, 28305, 28306, 28307, 28308, 28309, 28310, 28311, 28312, 28313, 28314, 28315, 28316, 28317, 28318, 28319, 28320, 28321, 28322, 28323, 28324, 28325, 28326, 28327, 28328, 28329, 28330, 28331, 28332) c6( ) c7(aW, nC, dV, aT); #c1(ARPP21) c2(NP_001254546) c3(CAMP-regulated phosphoprotein 21 isoform 3) c4(94637, 94638, 94639) c5(94640, 94641, 94642, 94643, 94644, 94645, 94646, 94647, 94648, 94649, 94650, 94651, 94652, 94653, 94654, 94655) c6( ) c7(aW, nC, dV, aT); #c1(ARPP21) c2(NP_001254548) c3(cAMP-regulated phosphoprotein 21 isoform 4) c4(94656) c5(94657, 94658, 94659, 94660, 94661, 94662, 94663, 94664, 94665, 94666) c6( ) c7(aW, nC, dV, aT); #c1(ARPP21) c2(NP_057384) c3(CAMP-regulated phosphoprotein 21 isoform 1) c4(190498) c5(190499, 190500, 190501, 190502, 190503, 190504) c6( ) c7(aW, nC, dV, aT); #c1(ARPP21) c2(XP_011531605) c3(CAMP-regulated phosphoprotein 21 isoform X4) c4(327744) c5( ) c6( ) c7(aW, nC, dV, aT); #c1(ARPP21) c2(XP_011531607) c3(CAMP-regulated phosphoprotein 21 isoform X7) c4(327745) c5( ) c6( ) c7(aW, nC, dV, aT); #c1(ARPP21) c2(XP_011531610) c3(CAMP-regulated phosphoprotein 21 isoform X10) c4(327746) c5( ) c6( ) c7(aW, nC, dV, aT); #c1(ARPP21) c2(XP_011531611) c3(cAMP-regulated phosphoprotein 21 isoform X11) c4(327747) c5( ) c6( ) c7(aW, nC, dV, aT); #c1(ARPP21) c2(XP_011531613) c3(cAMP-regulated phosphoprotein 21 isoform X13) c4( ) c5(327748) c6( ) c7(aW, nC, dV, aT); #c1(ARR3) c2(NP_004303) c3(arrestin-C) c4(144997, 144998, 144999) c5(145000, 145001, 145002, 145003, 145004, 145005, 145006, 145007, 145008, 145009, 145010, 145011, 145012, 145013, 145014, 145015, 145016, 145017) c6( ) c7(gh, gV, T, dA, qb, jh, fd, v, ci, q, ad, eA, eW, aT, dL, ae, af, eL, PP, s, hG, aW, Y, OX, oV, r, lo, t, cm, ck, kT, bP, mx, cj, iS, MJ, m, agl, gT, bt, B); #c1(ARRB1) c2(NP_004032) c3(beta-arrestin-1 isoform A) c4(142963, 142964) c5(142965, 142966, 142967, 142968, 142969, 142970, 142971, 142972, 142973, 142974, 142975, 142976) c6( ) c7(jh, JW, ai, eA, cd, bv, av, xE, aT, ji, yo, d, s, aW, Gv, fO, aq, iR, t, bz, eM, m, y); #c1(ARRB1) c2(NP_064647) c3(beta-arrestin-1 isoform B) c4(203458) c5(203459, 203460, 203461, 203462, 203463, 203464) c6( ) c7(jh, JW, ai, eA, cd, bv, av, xE, aT, ji, yo, d, s, aW, Gv, fO, aq, iR, t, bz, eM, m, y); #c1(ARRB2) c2(NP_001244258) c3(beta-arrestin-2 isoform 4) c4(92074) c5(92075, 92076, 92077, 92078, 92079, 92080) c6( ) c7(bL, T, jp, jh, ai, cd, ad, eW, HF, aT, K, ae, xa, yo, lR, iU, aW, Gv, fO, aq, HL, g, iR, GR, hq, cA, ao, de); #c1(ARRB2) c2(NP_001244259) c3 (beta-arrestin-2 isoform 5) c4(92081) c5(92082, 92083, 92084, 92085, 92086, 92087) c6( ) c7(bL, T, jp, jh, ai, cd, ad, eW, HF, aT, K, ae, xa, yo, lR, iU, aW, Gv, fO, aq, HL, g, iR, GR, hq, cA, ao, de); #c1(ARRB2) c2(NP_004304) c3(beta-arrestin-2 isoform 1) c4(145018) c5(145019, 145020, 145021, 145022, 145023, 145024, 145025, 145026, 145027, 145028, 145029, 145030) c6( ) c7(bL, T, jp, jh, ai, cd, ad, eW, HF, aT, K, ae, xa, yo, lR, iU, aW, Gv, fO, aq, HL, g, iR, GR, hq, cA, ao, de); #c1(ARRB2) c2(NP_945355) c3(beta-arrestin-2 isoform 2) c4(260899) c5(260900, 260901, 260902, 260903, 260904, 260905) c6( ) c7(bL, T, jp, jh, ai, cd, ad, eW, HF, aT, K, ae, xa, yo, lR, iU, aW, Gv, fO, aq, HL, g, iR, GR, hq, cA, ao, de); #c1(ARRB2) c2(XP_011522160) c3(beta-arrestin-2 isoform X1) c4(316761) c5( ) c6( ) c7(bL, T, jp, jh, ai, cd, ad, eW, HF, aT, K, ae, xa, yo, lR, iU, aW, Gv, fO, aq, HL, g, iR, GR, hq, cA, ao, de); #c1(ARRDC2) c2(NP_001020775) c3(arrestin domain-containing protein 2 isoform 2) c4(29109) c5(29110, 29111, 29112, 29113, 29114, 29115) c6( ) c7(j); #c1(ARRDC2) c2(NP_056498) c3(arrestin domain-containing protein 2 isoform 1) c4(187786) c5(187787, 187788, 187789, 187790, 187791, 187792) c6( ) c7(j); #c1(ARRDC3) c2(NP_065852) c3(arrestin domain-containing protein 3) c4(205905) c5(205906, 205907, 205908, 205909, 205910, 205911) c6( ) c7(aW, T, v, aT); #c1(ARRDC4) c2(NP_899232) c3(arrestin domain-containing protein 4) c4(258040, 258041) c5(258042, 258043, 258044, 258045, 258046, 258047, 258048, 258049, 258050, 258051, 258052, 258053) c6( ) c7(agJ, T, v, j, SE); #c1(ARSA) c2(NP_001078895) c3(arylsulfatase A isoform a precursor) c4(37966, 37967, 37968) c5(37969, 37970, 37971, 37972, 37973, 37974, 37975, 37976, 37977, 37978, 37979, 37980, 37981, 37982, 37983, 37984, 37985, 37986, 37987, 37988, 37989, 37990, 37991, 37992, 37993, 37994, 37995, 37996, 37997) c6( ) c7(bM, agO, agQ, T, dA, ob, ZF, iH, zs, agU, aw, NT, bO, bB, Ka, aj, j, bX, yU, V, q, aR, J, AK, agR, yZ, agV, K, ji, he, af, cp, agK, tN, yr, BO, agM, agP, agL, ge, r, dq, fe, agN, iV, f, bz, zV, agS, ac, Rd, bE, U, Lq, gT, agT); #c1(ARSA) c2(XP_011528992) c3(arylsulfatase A isoform X1) c4( ) c5(324358, 324359, 324360, 324361, 324362) c6( ) c7(bM, agO, agQ, T, dA, ob, ZF, iH, zs, agU, aw, NT, bO, bB, Ka, aj, j, bX, yU, V, q, aR, J, AK, agR, yZ, agV, K, ji, he, af, cp, agK, tN, yr, BO, agM, agP, agL, ge, r, dq, fe, agN, iV, f, bz, zV, agS, ac, Rd, bE, U, Lq, gT, agT); #c1(ARSB) c2(NP_000037) c3(arylsulfatase B isoform 1 precursor) c4(477, 478) c5(479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578) c6(579, 580, 581, 582, 583) c7(Rd, kg, iU, oe, ao, dq, Lq, j, ade, agW, dr); #c1(ARSB) c2(NP_942002) c3(arylsulfatase B isoform 2 precursor) c4(260405) c5(260406, 260407, 260408, 260409, 260410, 260411, 260412, 260413) c6(260414, 260415) c7(Rd, kg, iU, oe, ao, dq, Lq, j, ade, agW, dr); #c1(ARSB) c2(XP_006714678) c3(arylsulfatase B isoform X6) c4(287996) c5( ) c6( ) c7(Rd, kg, iU, oe, ao, dq, Lq, j, ade, agW, dr); #c1(ARSB) c2(XP_011541695) c3(arylsulfatase B isoform X5) c4(340220) c5( ) c6( ) c7(Rd, kg, iU, oe, ao, dq, Lq, j, ade, agW, dr); #c1(ARSD) c2(NP_001660) c3(arylsulfatase D precursor) c4(119833) c5(119834, 119835, 119836, 119837, 119838, 119839) c6( ) c7(hl, ahf, ahc, acI, RX, ha, VT, fF, bS, j, ct, nA, agY, aR, xt, agd, no, KN, h, adm, bK, ahg, jj, rE, rz, ahb, ahd, Gv, ahe, nf, gl, Ku, aha, Z, QK, bN, hq, ac, agX, agZ, yA, bD, jr, qc); #c1(ARSE) c2(NP_001269557) c3(arylsulfatase E isoform 1) c4(101953) c5(101954, 101955, 101956, 101957, 101958, 101959) c6( ) c7(es, Lf, ahj, ahh, bu, ahi); #c1(ARSE) c2(NP_001269560) c3(arylsulfatase E isoform 3) c4(101979) c5(101980, 101981, 101982, 101983, 101984, 101985) c6( ) c7(es, Lf, ahj, ahh, bu, ahi); #c1(ARSE) c2(XP_005274575) c3(arylsulfatase E isoform X2) c4(285159, 285160) c5(285161, 285162) c6( ) c7(es, Lf, ahj, ahh, bu, ahi); #c1(ARSE) c2(XP_005274576) c3(arylsulfatase E isoform X1) c4( ) c5(285163, 285164, 285165, 285166, 285167, 285168, 285169, 285170, 285171, 285172) c6( ) c7(es, Lf, ahj, ahh, bu, ahi); #c1(ARSF) c2(NP_004033) c3(arylsulfatase F precursor) c4( ) c5(142977, 142978, 142979, 142980, 142981, 142982, 142983, 142984, 142985, 142986, 142987, 142988) c6( ) c7(es, T, av, lg, eM, iA, aW, ds, iU, ahk, lb, ff, aT, kf, lo, en, cm, on); #c1(ARSF) c2(XP_011543824) c3(arylsulfatase F isoform X1) c4(342959) c5(342960, 342961) c6( ) c7(es, T, av, lg, eM, iA, aW, ds, iU, ahk, lb, ff, aT, kf, lo, en, cm, on); #c1(ARSG) c2(NP_055775) c3(arylsulfatase G) c4(183406, 183407) c5(183408, 183409, 183410, 183411, 183412, 183413, 183414, 183415, 183416, 183417, 183418, 183419, 183420, 183421, 183422, 183423) c6( ) c7(cd, c, cz); #c1(ARSH) c2(NP_001011719) c3(arylsulfatase H) c4(24800) c5(24801, 24802, 24803, 24804, 24805, 24806) c6( ) c7(T, ahj, agG, agV, ci, cm); #c1(ARSI) c2(NP_001012301) c3(arylsulfatase 1 precursor) c4(24854, 24855, 24856) c5(24857, 24858, 24859, 24860, 24861, 24862, 24863, 24864, 24865, 24866, 24867, 24868, 24869, 24870, 24871, 24872, 24873, 24874) c6( ) c7(ww, BO, tb, hq); #c1(ARSJ) c2(NP_078866) c3(arylsulfatase J precursor) c4(215839, 215840) c5(215841, 215842, 215843, 215844, 215845, 215846) c6( ) c7(oe, ac); #c1(ARSJ) c2(XP_005263269) c3(arylsulfatase J isoform X1) c4(278373) c5(278374, 278375, 278376, 278377, 278378, 278379) c6( ) c7(oe, ac); #c1(ARSJ) c2(XP_011530540) c3 (arylsulfatase J isoform X2) c4( ) c5(326475) c6( ) c7(oe, ac); #c1(ARSJ) c2(XP_011530544) c3(arylsulfatase J isoform X4) c4( ) c5(326476, 326477, 326478, 326479, 326480, 326481) c6( ) c7(oe, ac); #c1(ARSK) c2(NP_937793) c3 (arylsulfatase K precursor) c4(259083, 259084) c5(259085, 259086, 259087, 259088, 259089, 259090, 259091, 259092, 259093, 259094, 259095, 259096) c6( ) c7(Lq); #c1(ART1) c2(NP_004305) c3(GPI-linked NAD(P)(+)-arginine ADP-ribosyltransferase 1 precursor) c4(145031) c5(145032, 145033, 145034, 145035, 145036, 145037) c6( ) c7(Y, ff, aE, en); #c1(ART3) c2(NP_001123488) c3(ecto-ADP-ribosyltransferase 3 isoform a precursor) c4(47175) c5(47/76, 47177, 47/78, 47/79, 47180, 47181) c6( ) c7(vH); #c1(ART3) c2(NP_001123489) c3(ecto-ADP-ribosyltransferase 3 isoform c precursor) c4(47182) c5(47183, 47184, 47185, 47186, 47187, 47188) c6( ) c7(vH); #c1(ART3) c2(XP_005263061) c3(ecto-ADP-ribosyltransferase 3 isoform X11) c4( ) c5(278277, 278278, 278279, 278280, 278281, 278282, 278283, 278284, 278285, 278286, 278287, 278288, 278289, 278290, 278291, 278292, 278293, 278294, 278295, 278296, 278297, 278298, 278299, 278300) c6( ) c7(vH); #c1(ART3) c2(XP_006714281) c3(ecto-ADP-ribosyltransferase 3 isoform X6) c4(287671, 287672, 287673, 287674) c5( ) c6( ) c7(vH); #c1(ART4) c2(NP_066549) c3(ecto-ADP-ribosyltransferase 4 precursor) c4(207353, 207354) c5(207355, 207356, 207357, 207358, 207359, 207360, 207361, 207362, 207363, 207364, 207365, 207366) c6( ) c7(ck, T, kP, jh, cj, aA, aF, m, B); #c1(ARVCF) c2(XP_005261300) c3(armadillo repeat protein deleted in velo-cardio-facial syndrome isoform X5) c4(277288) c5(277289, 277290, 277291, 277292, 277293, 277294) c6( ) c7(ae, ac, iX, dL, dy, bX, fq, cd, gF, ad); #c1(ARVCF) c2(XP_006724310) c3(armadillo repeat protein deleted in velo-cardio-facial syndrome isoform X9) c4(295919) c5(295920, 295921, 295922, 295923, 295924, 295925, 295926, 295927, 295928, 295929) c6( ) c7(ae, ac, iX, dL, dy, bX, fq, cd, gF, ad); #c1(ARVCF) c2(XP_006724311) c3(armadillo repeat protein deleted in velo-cardio-facial syndrome isoform X10) c4(295930) c5(295931, 295932, 295933, 295934, 295935, 295936) c6( ) c7(ae, ac, iX, dl, dy, bX, fq, cd, gF, ad); #c1(ARVCF) c2(XP_011528482) c3(armadillo repeat protein deleted in velo-cardio-facial syndrome isoform X1) c4(323590) c5(323591, 323592, 323593, 323594, 323595, 323596) c6( ) c7(ae, ac, iX, dl, dy, bX, fq, cd, gF, ad); #c1(ARX) c2(NP_620689) c3(homeobox protein ARX) c4(238044) c5(238045, 238046, 238047, 238048, 238049, 238050, 238051) c6( ) c7(bL, ul, qx, ha, aht, nA, aR, ahs, nd, aU, ahr, aho, lm, ahu, cC, yr, ahl, ahm, aeq, FD, nf, ahq, xw, bN, ahn, ahp, WO, FB); #c1(AS3MT) c2(NP_065733) c3(arsenite methyltransferase) c4(205123) c5(205124, 205125, 205126, 205127, 205128, 205129) c6( ) c7(T, dD, hk, ea, e, eL, aj, dL, cS, K, lQ, j, dQ, Pk, ahv, eq, J); #c1(ASAHI) c2(NP_001120977) c3(acid ceramidase isoform c) c4(45030) c5(45031, 45032, 45033, 45034, 45035, 45036, 45037) c6( ) c7(T, AS, ml, adi, bS, v, ct, nA, yU, ad, acX, ahy, hd, mh, aE, db, RN, xW, aT, kf, yZ, Fx, K, EL, ae, ahA, cp, aW, Y, OX, aq, ahx, Z, bz, dr, EJ, ck, nB, aem, gX, ahw, ahz, Lq, du, agT, B); #c1(ASAHI) c2(NP_004306) c3 (acid ceramidase isoform b) c4(145038, 145039) c5(145040, 145041, 145042, 145043, 145044, 145045, 145046) c6( ) c7(T, AS, ml, adi, bS, v, ct, nA, yU, ad, acX, ahy, hd, mh, aE, db, RN, xW, aT, kf, yZ, Fx, K, EL, ae, ahA, cp, aW, Y, OX, aq, ahx, Z, bz, dr, EJ, ck, nB, aem, gX, ahw, ahz, Lq, du, agT, B); #c1(ASAH1) c2(NP_808592) c3(acid ceramidase isoform a preproprotein) c4(252738, 252739) c5(252740, 252741, 252742, 252743, 252744, 252745, 252746, 252747, 252748, 252749, 252750, 252751, 252752, 252753, 252754, 252755, 252756, 252757, 252758, 252759, 252760, 252761, 252762, 252763, 252764, 252765, 252766, 252767, 252768, 252769, 252770, 252771, 252772, 252773, 252774, 252775, 252776, 252777, 252778, 252779, 252780, 252781, 252782, 252783, 252784, 252785, 252786, 252787, 252788, 252789, 252790, 252791, 252792, 252793, 252794, 252795, 252796, 252797, 252798, 252799, 252800, 252801, 252802, 252803, 252804, 252805, 252806, 252807, 252808, 252809, 252810, 252811, 252812, 252813, 252814, 252815, 252816, 252817, 252818, 252819, 252820, 252821, 252822, 252823, 252824, 252825, 252826, 252827, 252828, 252829, 252830, 252831, 252832, 252833, 252834, 252835, 252836, 252837, 252838, 252839, 252840, 252841, 252842, 252843, 252844, 252845, 252846, 252847, 252848, 252849, 252850, 252851, 252852, 252853, 252854, 252855, 252856, 252857, 252858, 252859, 252860, 252861, 252862, 252863, 252864, 252865, 252866, 252867, 252868, 252869, 252870, 252871, 252872, 252873, 252874, 252875, 252876, 252877, 252878, 252879, 252880, 252881, 252882, 252883, 252884, 252885, 252886, 252887, 252888, 252889, 252890, 252891) c6( ) c7(T, AS, ml, adi, bS, v, ct, nA, yU, ad, acX, ahy, hd, mh, aE, db, RN, xW, aT, kf, yZ, Fx, K, EL, ae, ahA, cp, aW, Y, OX, aq, ahx, Z, bz, dr, EJ, ck, nB, aem, gX, ahw, ahz, Lq, du, agT, B); #c1(ASAHI) c2(XP_005273561) c3(acid ceramidase isoform X1) c4( ) c5(284675, 284676, 284677, 284678, 284679, 284680) c6( ) c7(T, AS, ml, adi, bS, v, ct, nA, yU, ad, acX, ahy, hd, mh, aE, db, RN, xW, aT, kf, yZ, Fx, K, EL, ae, ahA, cp, aW, Y, OX, aq, ahx, Z, bz, dr, EJ, ck, nB, aem, gX, ahw, ahz, Lq, du, agT, B); #c1(ASAH2) c2(NP_001137446) c3(neutral ceramidase isoform b) c4(55765, 55766) c5(55767, 55768, 55769, 55770, 55771, 55772, 55773, 55774, 55775, 55776, 55777, 55778) c6( ) c7(IZ, fm, oe, hl, EQ, fe, ao); #c1(ASAH2) c2(XP_011538272) c3(neutral ceramidase isoform X1) c4(335909, 335910, 335911, 335912, 335913) c5(335914, 335915, 335916, 335917, 335918, 335919, 335920, 335921, 335922, 335923, 335924, 335925, 335926, 335927, 335928, 335929, 335930, 335931, 335932, 335933, 335934, 335935, 335936, 335937) c6( ) c7(IZ, fm, oe, hl, EQ, fe, ao); #c1(ASAH2) c2(XP_011538274) c3(neutral ceramidase isoform X3) c4(335938) c5(335939, 335940, 335941, 335942, 335943, 335944, 335945, 335946) c6( ) c7(IZ, fm, oe, hl, EQ, fe, ao); #c1(ASAP1) c2(NP_060952) c3(arf-GAP with SH3 domain, ANK repeat and PH domain-containing protein 1 isoform 1) c4(198860) c5(198861, 198862, 198863, 198864, 198865, 198866, 198867, 198868, 198869, 198870) c6( ) c7(T, dA, iW, aj, v, V, cd, q, ad, aT, ae, d, aW, Y, r, uF, iK, iP, ck, wq, dT, zG, zl, ao); #c1(ASAP1) c2(XP_011515354) c3(arf-GAP with SH3 domain, ANK repeat and PH domain-containing protein 1 isoform X1) c4(308195) c5(308196, 308197, 308198, 308199, 308200, 308201) c6( ) c7(T, dA, iW, aj, v, V, cd, q, ad, aT, ae, d, aW, Y, r, uF, iK, iP, ck, wq, dT, zG, zl, ao); #c1(ASAP2) c2(NP_001128663) c3(arf-GAP with SH3 domain, ANK repeat and PH domain-containing protein 2 isoform b) c4(50496) c5(50497, 50498, 50499, 50500, 50501, 50502) c6( ) c7(ck, d, dA, iW, r, wq, dT, aj, v, zG, uF, zl, V, cd, ao); #c1(ASAP2) c2(NP 003878) c3(arf-GAP with SH3 domain, ANK repeat and PH domain-containing protein 2 isoform a) c4(141743) c5(141744, 141745, 141746, 141747, 141748, 141749) c6( ) c7(ck, d, dA, iW, r, wq, dT, aj, v, zG, uF, zl, V, cd, ao); #c1(ASAP2) c2(XP_011508705) c3(arf-GAP with SH3 domain, ANK repeat and PH domain-containing protein 2 isoform X1) c4( ) c5(299318) c6( ) c7(ck, d, dA, iW, r, wq, dT, aj, v, zG, uF, zl, V, cd, ao); #c1(ASB1) c2(NP_001035535) c3(ankyrin repeat and SOCS box protein 1) c4(32880) c5(32881, 32882, 32883, 32884, 32885, 32886) c6( ) c7(hH, bK); #c1(ASB10) c2(NP_001135931) c3(ankyrin repeat and SOCS box protein 10 isoform 1) c4(53577, 53578) c5(53579, 53580, 53581, 53582, 53583, 53584, 53585, 53586, 53587, 53588, 53589, 53590) c6( ) c7(cZ, pZ, dH, dd, dw, ahB); #c1(ASB10) c2(NP_001135932) c3(ankyrin repeat and SOCS box protein 10 isoform 2) c4(53591, 53592) c5(53593, 53594, 53595, 53596, 53597, 53598, 53599, 53600, 53601, 53602, 53603) c6( ) c7(cZ, pZ, dH, dd, dw, ahB); #c1(ASB10) c2(NP_543147) c3(ankyrin repeat and SOCS box protein 10 isoform 3) c4(233458) c5(233459, 233460, 233461, 233462, 233463, 233464) c6( ) c7(cZ, pZ, dH, dd, dw, ahB); #c1(ASB13) c2(NP_078977) c3(ankyrin repeat and SOCS box protein 13) c4(216367) c5(216368, 216369, 216370, 216371, 216372, 216373) c6( ) c7(cF); #c1(ASB15) c2(XP_005250203) c3(ankyrin repeat and SOCS box protein 15 isoform X1) c4(270173, 270174) c5(270175, 270176, 270177, 270178, 270179, 270180, 270181, 270182, 270183, 270184, 270185, 270186, 270187, 270188, 270189, 270190, 270191, 270192, 270193, 270194, 270195, 270196, 270197, 270198, 270199) c6( ) c7(fr); #c1(ASB15) c2(XP_011514118) c3(ankyrin repeat and SOCS box protein 15 isoform X2) c4(306704, 306705) c5(306706, 306707, 306708, 306709) c6( ) c7(fr); #c1(ASB15) c2(XP_011514123) c3 (ankyrin repeat and SOCS box protein 15 isoform X3) c4( ) c5(306710, 306711, 306712, 306713, 306714, 306715) c6( ) c7(fr); #c1(ASB15) c2(XP_011514124) c3(ankyrin repeat and SOCS box protein 15 isoform X4) c4( ) c5(306716, 306717, 306718, 306719, 306720) c6( ) c7(fr); #c1(ASB18) c2(NP_997721) c3(ankyrin repeat and SOCS box protein 18) c4(265769, 265770) c5(265771, 265772, 265773, 265774, 265775, 265776, 265777, 265778, 265779, 265780, 265781, 265782) c6( ) c7(fq); #c1(ASB2) c2(NP_057234) c3(ankyrin repeat and SOCS box protein 2 isoform 2) c4(189627) c5(189628, 189629, 189630, 189631, 189632, 189633) c6( ) c7(ck, kS, j, jj, iT); #c1(ASB2) c2(XP_006725228) c3(ankyrin repeat and SOCS box protein 2 isoform X1) c4(296732, 296733, 296734) c5(296735, 296736, 296737, 296738, 296739, 296740, 296741, 296742, 296743, 296744, 296745, 296746) c6( ) c7(ck, kS, j, jj, lT); #c1(ASB2) c2(XP_011544948) c3 (ankyrin repeat and SOCS box protein 2 isoform X3) c4( ) c5(343939, 343940, 343941, 343942, 343943, 343944) c6( ) c7(ck, kS, j, jj, iT); #c1(ASB6) c2(NP_060343) c3(ankyrin repeat and SOCS box protein 6 isoform 1) c4(195507, 195508) c5(195509, 195510, 195511, 195512, 195513, 195514, 195515, 195516, 195517, 195518, 195519, 195520, 195521) c6( ) c7(cz); #c1(ASB6) c2(NP_821066) c3(ankyrin repeat and SOCS box protein 6 isoform 2) c4(253158) c5(253159, 253160, 253161, 253162, 253163) c6( ) c7(cz); #c1(ASB7) c2(NP_078984) c3(ankyrin repeat and SOCS box protein 7 isoform 1) c4(216424) c5(216425, 216426, 216427, 216428, 216429, 216430) c6( ) c7(j); #c1(ASB7) c2(NP_937886) c3(ankyrin repeat and SOCS box protein 7 isoform 2) c4(259443) c5(259444, 259445, 259446, 259447, 259448, 259449) c6( ) c7(j); #c1(ASCC1) c2(NP_001185727) c3(activating signal cointegrator 1 complex subunit 1 isoform b) c4(76120, 76121, 76122, 76123) c5(76124, 76125, 76126, 76127, 76128, 76129, 76130, 76131, 76132, 76133, 76134, 76135, 76136, 76137, 76138, 76139, 76140, 76141, 76142, 76143, 76144, 76145, 76146) c6( ) c7(T, pf, aw, abM, rf, aA, bB, ti, j, eA, eW, oT, agd, aE, aT, cU, cy, h, bK, K, zy, he, ae, cp, UF, eG, aW, Y, Gv, az, gi, LZ, agb, gl, aga, aF, as, iV, t, cm, ek, LB, KT, age, eU, agc, eM, bE, w, dO, at, m, fD, Uw, fy, bt, y); #c1(ASCC1) c2(NP_00185728) c3(activating signal cointegrator 1 complex subunit 1 isoform a) c4(76147) c5(76148, 76149, 76150, 76151, 76152, 76153, 76154, 76155, 76156, 76157) c6( ) c7(T, pf, aw, abM, rf, aA, bB, ti, j, eA, eW, oT, agd, aE, aT, cU, cy, h, bK, K, zy, he, ae, cp, UF, eG, aW, Y, Gv, az, gi, LZ, agb, gl, aga, aF, as, iV, t, cm, ek, LB, KT, age, eU, agc, eM, bE, w, do, at, m, fD, Uw, fy, bt, y); #c1(ASCC1) c2(XP_011538144) c3(activating signal cointegrator 1 complex subunit 1 isoform X3) c4(335745, 335746) c5(335747, 335748, 335749, 335750, 335751, 335752, 335753, 335754, 335755, 335756, 335757, 335758) c6( ) c7(T, pf, aw, abM, rf, aA, bB, ti, j, eA, eW, oT, agd, aE, aT, cU, cy, h, bK, K, zy, he, ae, cp, UF, eG, aW, Y, Gv, az, gi, LZ, agb, gl, aga, aF, as, iV, t, cm, ek, LB, KT, age, eU, agc, eM, bE, w, do, at, m, fD, Uw, fy, bt, y); #c1(ASCC1) c2(XP_011538145) c3(activating signal cointegrator 1 complex subunit 1 isoform X4) c4(335759, 335760) c5(335761) c6( ) c7(T, pf, aw, abM, rf, aA, bB, ti, j, eA, eW, oT, agd, aE, aT, cU, cy, h, bK, K, zy, he, ae, cp, UF, eG, aW, Y, Gv, az, gi, LZ, agb, gl, aga, aF, as, iV, t, cm, ek, LB, KT, age, eU, agc, eM, bE, w, do, at, m, fD, Uw, fy, bt, y); #c1(ASCC1) c2(XP_011538146) c3 (activating signal cointegrator 1 complex subunit 1 isoform X5) c4( ) c5(335762, 335763, 335764, 335765, 335766, 335767, 335768, 335769, 335770, 335771, 335772, 335773, 335774, 335775, 335776, 335777, 335778, 335779, 335780, 335781, 335782, 335783, 335784, 335785) c6( ) c7(T, pf, aw, abM, rf, aA, bB, ti, j, eA, eW, oT, agd, aE, aT, cU, cy, h, bK, K, zy, he, ae, cp, UF, eG, aW, Y, Gv, az, gi, LZ, agb, gl, aga, aF, as, iV, t, cm, ek, LB, kT, age, eU, agc, eM, bE, w, do, at, m, fD, Uw, fy, bt, y); #c1(ASCC2) c2(NP_001229835) c3(activating signal cointegrator 1 complex subunit 2 isoform 2) c4(85396) c5(85397, 85398, 85399, 85400, 85401, 85402) c6( ) c7(iM, je, jh, kH, ahC, bB, fd, qN, ae, ad); #c1(ASCC2) c2(NP_115580) c3(activating signal cointegrator 1 complex subunit 2 isoform 1) c4(223269, 223270) c5(223271, 223272, 223273, 223274, 223275, 223276, 223277, 223278, 223279, 223280) c6( ) c7(iM, je, jh, kH, ahC, bB, fd, qN, ae, ad); #c1(ASCC2) c2(XP_011528749) c3(activating signal cointegrator 1 complex subunit 2 isoform X3) c4( ) c5(324054, 324055, 324056, 324057, 324058, 324059) c6( ) c7(iM, je, jh, kH, ahC, bB, fd, qN, ae, ad); #c1(ASCC3) c2(NP_001271200) c3(activating signal cointegrator 1 complex subunit 3 isoform c) c4(103571) c5(103572, 103573, 103574, 103575, 103576, 103577) c6( ) c7(Kc, T, fM, ac, r, ey, nA, fq, q, ahD); #c1(ASCC3) c2(NP_006819) c3(activating signal cointegrator 1 complex subunit 3 isoform a) c4(168379) c5(168380, 168381, 168382, 168383, 168384, 168385) c6( ) c7(Kc, T, fM, ac, r, ey, nA, fq, q, ahD); #c1(ASCC3) c2(NP_071374) c3(activating signal cointegrator 1 complex subunit 3 isoform b) c4(211230) c5(211231, 211232, 211233, 211234, 211235, 211236) c6( ) c7(Kc, T, fM, ac, r, ey, nA, fq, q, ahD); #c1(ASCL1) c2(NP_004307) c3(achaete-scute homolog 1) c4(145047) c5(145048, 145049, 145050, 145051, 145052, 145053, 145054) c6( ) c7(T, ha, qy, DE, BH, ci, nA, yp, pz, Fc, eA, aR, ad, av, ft, hd, nv, pX, ahl, aT, Ou, an, ahk, ahE, Yi, jf, s, ahG, aW, Y, AD, ff, ahF, ahJ, ahI, en, g, as, cm, bz, ac, eM, nB, nG, ahH, cB, iu); #c1(ASCL2) c2(NP_005161) c3(achaete-scute homolog 2) c4(153528) c5(153529, 153530, 153531, 153532, 153533, 153534, 153535) c6( ) c7(T, B, CN, s, X, hL, r, aA, aF, ci, cl, nJ, q); #c1(ASCL4) c2(NP_982260) c3(achaete-scute homolog 4) c4(263234) c5(263235, 263236, 263237, 263238, 263239, 263240) c6( ) c7(aj); #c1(ASF1A) c2(NP_054753) c3 (histone chaperone ASF1A) c4(177619) c5(177620, 177621, 177622, 177623, 177624, 177625) c6( ) c7(zw, xE, hA, Y, aT, y, fb, ai); #c1(ASF1B) c2(NP_060624) c3(histone chaperone ASF1B) c4(196884) c5(196885, 196886, 196887, 196888, 196889, 196890) c6( ) c7(aW, aT); #c1(ASH1L) c2(XP_006711513) c3(histone-lysine N-methyltransferase ASH1L isoform X1) c4(286047, 286048) c5(286049, 286050, 286051, 286052, 286053, 286054, 286055, 286056, 286057, 286058, 286059, 286060, 286061) c6( ) c7(ft, ff, rx, en, cm, bz); #c1(ASH2L) c2(NP_001098684) c3(set1/Ash2 histone methyltransferase complex subunit ASH2 isoform b) c4( ) c5(41485, 41486, 41487, 41488, 41489, 41490) c6( ) c7(aW, gv, B, ci, aT); #c1(ASH2L) c2(NP_001269201) c3(set1/Ash2 histone methyltransferase complex subunit ASH2 isoform d) c4( ) c5(100759, 100760, 100761, 100762, 100763) c6( ) c7(aW, gv, B, ci, aT); #c1(ASH2L) c2(NP_004665) c3(set1/Ash2 histone methyltransferase complex subunit ASH2 isoform a) c4(148685, 148686, 148687) c5(148688, 148689, 148690, 148691, 148692, 148693, 148694, 148695) c6( ) c7(aW, gv, B, ci, aT); #c1(ASH2L) c2(XP_006716476) c3(set1/Ash2 histone methyltransferase complex subunit ASH2 isoform X3) c4(290348) c5(290349, 290350, 290351, 290352, 290353, 290354, 290355, 290356, 290357, 290358) c6( ) c7(aW, gv, B, ci, aT); #c1(ASIC1) c2(NP_001086) c3(acid-sensing ion channel 1 isoform b) c4(38241) c5(38242, 38243, 38244, 38245, 38246, 38247) c6( ) c7(Mo, ahN, d, ac, ll, Kv, qK, ahM, cz, cy, lN, eA, lC, abC, V, cd, eu); #c1(ASIC1) c2(NP_001243759) c3(acid-sensing ion channel 1 isoform c) c4(91674) c5(91675, 91676, 91677, 91678, 91679, 91680) c6( ) c7(Mo, ahN, d, ac, ll, Kv, qK, ahM, cz, cy, lN, eA, lC, abC, V, cd, eu); #c1(ASIC1) c2(NP_064423) c3(acid-sensing ion channel 1 isoform a) c4(202415) c5(202416, 202417, 202418, 202419, 202420, 202421) c6( ) c7(Mo, ahN, d, ac, ll, Kv, qK, ahM, cz, cy, lN, eA, lC, abC, V, cd, eu); #c1(ASIC2) c2(NP_001085) c3(acid-sensing ion channel 2 isoform MDEG1) c4(38227, 38228) c5(38229, 38230, 38231, 38232, 38233, 38234, 38235, 38236, 38237, 38238, 38239, 38240) c6( ) c7(nD, T, cF, d, ac, tb, aE, dM, cm, ww, j, bK, an, gT, V, fq, eA, bN); #c1(ASIC2) c2(NP_899233) c3(acid-sensing ion channel 2 isoform MDEG2) c4(258054) c5(258055, 258056, 258057, 258058, 258059, 258060) c6( ) c7(nD, T, cF, d, ac, tb, aE, dM, cm, ww, j, bK, an, gT, V, fq, eA, bN); #c1(ASIC3) c2(NP_004760) c3(acid-sensing ion channel 3 isoform a) c4(149379) c5(149380, 149381, 149382, 149383, 149384, 149385) c6( ) c7(qX, ahO, xZ, ahP, cz, ahQ, mf); #c1(ASIC3) c2(NP_064717) c3(acid-sensing ion channel 3 isoform b) c4(203552) c5(203553, 203554, 203555, 203556, 203557, 203558) c6( ) c7(qX, ahO, xZ, ahP, cz, ahQ, mf); #c1(ASIC3) c2(NP_064718) c3(acid-sensing ion channel 3 isoform c) c4(203559) c5(203560, 203561, 203562, 203563, 203564, 203565) c6( ) c7(qX, ahO, xZ, ahP, cz, ahQ, mf); #c1(ASIC4) c2(NP_878267) c3(acid-sensing ion channel 4) c4(257064) c5(257065, 257066, 257067, 257068, 257069, 257070) c6( ) c7(ahR, xw, aR); #c1(ASIC4) c2(XP_005246728) c3(acid-sensing ion channel 4 isoform X1) c4(267390) c5(267391, 267392, 267393, 267394, 267395, 267396) c6( ) c7(ahR, xw, aR); #c1(ASIC5) c2(NP_059115) c3(acid-sensing ion channel 5) c4(193113) c5(193114, 193115, 193116, 193117, 193118, 193119) c6( ) c7(ao, MF, ahS, qa, cz); #c1(ASIP) c2(XP_005260469) c3(agouti-signaling protein isoform X2) c4( ) c5(276566) c6( ) c7(sc, T, yk, n, v, j, o, x, l, u, afd, h, ahU, ahT, A, Y, ff, cr, en, t, ahV, sS, Dy, c, hk, w, m, k, y, B, p); #c1(ASIP) c2(XP_011527125) c3(agouti-signaling protein isoform X1) c4(321628) c5(321629, 321630, 321631, 321632, 321633) c6( ) c7(sc, T, yk, n, v, j, o, x, l, u, afQ, h, ahU, ahT, A, Y, ff, cr, en, t, ahV, sS, Dy, c, hk, w, m, k, y, B, p); #c1(ASL) c2(NP_000039) c3(argininosuccinate lyase isoform 1) c4(584) c5(585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732) c6(733) c7(Na, ahW, T, afC, d, JZ, wz, Lv, Ka, aq, Nc, K, jo, nA, iR, B, bN); #c1(ASL) c2(NP_001020115) c3(argininosuccinate lyase isoform 2) c4(28217) c5(28218, 28219, 28220, 28221, 28222, 28223) c6( ) c7(Na, ahW, T, afC, d, JZ, wz, Lv, Ka, aq, Nc, K, jo, nA, iR, B, bN); #c1(ASL) c2(NP_001020117) c3(argininosuccinate lyase isoform 3) c4(28224, 28225) c5(28226, 28227, 28228, 28229, 28230, 28231, 28232, 28233, 28234, 28235, 28236, 28237) c6( ) c7(Na, ahW, T, afC, d, JZ, wz, Lv, Ka, aq, Nc, K, jo, nA, iR, B, bN); #c1(ASMT) c2(NP_001164510) c3(acetylserotonin O-methyltransferase isoform 2) c4(66980) c5(66981, 66982, 66983, 66984, 66985, 66986, 66987, 66988, 66989, 66990, 66991, 66992) c6( ) c7(bP, rz, ahX, MU, bS, bK, ct, Z, he, bN); #c1(ASMT) c2(NP_004034) c3(acetylserotonin O-methyltransferase isoform 1) c4(142989, 142990, 142991) c5(142992, 142993, 142994, 142995, 142996, 142997, 142998, 142999, 143000, 143001, 143002, 143003) c6( ) c7(bP, rz, ahX, MU, bS, bK, ct, Z, he, bN); #c1(ASMTL) c2(NP_001166945) c3(N-acetylserotonin O-methyltransferase-like protein isoform 3) c4(68693) c5(68694, 68695, 68696, 68697, 68698, 68699) c6( ) c7(bt); #c1(ASMTL) c2(NP_004183) c3(N-acetylserotonin O-methyltransferase-like protein isoform 1) c4(144142, 144143, 144144) c5(144145, 144146, 144147, 144148, 144149, 144150, 144151, 144152, 144153, 144154, 144155, 144156, 144157, 144158, 144159, 144160, 144161, 144162) c6( ) c7(bt); #c1(ASNA1) c2(NP_004308) c3(ATPase ASNA1) c4(145055) c5(145056, 145057, 145058, 145059, 145060, 145061, 145062, 145063, 145064, 145065) c6( ) c7(AK, he); #c1(ASNS) c2(NP_001171546) c3(asparagine synthetase [glutamine-hydrolyzing] isoform b) c4(70554) c5(70555, 70556, 70557, 70558, 70559) c6( ) c7(ck, x, T, aE, hb, NT, aq, ahY, K, m, k, du, t, B, ahZ); #c1(ASNS) c2(NP_001171548) c3(asparagine synthetase [glutamine-hydrolyzing] isoform c) c4(70560) c5(70561, 70562, 70563, 70564, 70565, 70566, 70567, 70568, 70569) c6( ) c7(ck, x, T, aE, hb, NT, aq, ahY, K, m, k, du, t, B, ahZ); #c1(ASNS) c2(NP_597680) c3(asparagine synthetase [glutamine-hydrolyzing] isoform a) c4(234983, 234984, 234985, 234986, 234987, 234988, 234989) c5(234990, 234991, 234992, 234993, 234994, 234995, 234996, 234997, 234998, 234999, 235000, 235001, 235002, 235003, 235004, 235005, 235006, 235007, 235008, 235009, 235010, 235011, 235012, 235013, 235014, 235015, 235016, 235017, 235018, 235019, 235020, 235021, 235022, 235023, 235024, 235025, 235026, 235027, 235028, 235029, 235030, 235031, 235032, 235033, 235034, 235035, 235036, 235037, 235038, 235039, 235040, 235041) c6( ) c7(ck, x, T, aE, hb, NT, aq, ahY, K, m, k, du, t, B, ahZ); #c1(ASPA) c2(XP_006721590) c3(aspartoacylase isoform X1) c4(294012) c5(294013, 294014, 294015, 294016, 294017, 294018, 294019, 294020, 294021, 294022, 294023, 294024, 294025, 294026, 294027, 294028, 294029, 294030, 294031, 294032, 294033, 294034, 294035, 294036, 294037, 294038, 294039, 294040, 294041, 294042, 294043, 294044, 294045, 294046, 294047, 294048, 294049, 294050, 294051, 294052, 294053, 294054, 294055, 294056, 294057, 294058, 294059, 294060, 294061, 294062, 294063, 294064, 294065, 294066, 294067, 294068, 294069, 294070, 294071, 294072, 294073, 294074, 294075, 294076, 294077, 294078, 294079, 294080, 294081, 294082, 294083, 294084, 294085, 294086, 294087, 294088, 294089, 294090, 294091, 294092, 294093, 294094, 294095, 294096, 294097, 294098, 294099, 294100, 294101, 294102, 294103, 294104, 294105, 294106, 294107, 294108) c6( ) c7(aib, n, v, j, eA, o, x, l, u, h, cb, A, aia, cm, t, dr, c, w, U, aic, k, m, y, sH, p); #c1(ASPG) c2(NP_001073933) c3(60 kDa lysophospholipase) c4(36574, 36575) c5(36576, 36577, 36578, 36579, 36580, 36581, 36582, 36583, 36584, 36585, 36586, 36587, 36588, 36589, 36590, 36591) c6(36592, 36593, 36594, 36595) c7(ck, x, cT, l, fX, T, ac, hG, oe, oW, cz, jh, aid, DU, k, B); #c1(ASPG) c2(XP_005267647) c3(60 kDa lysophospholipase isoform X4) c4(281179, 281180) c5(281181, 281182, 281183, 281184, 281185, 281186, 281187, 281188, 281189, 281190, 281191, 281192, 281193, 281194, 281195, 281196) c6(281197, 281198, 281199, 281200) c7(ck, x, cT, l, fX, T, ac, hG, oe, oW, cz, jh, aid, DU, k, B); #c1(ASPH) c2(NP_001158222) c3(aspartyl/asparaginyl beta-hydroxylase isoform f) c4(63450) c5(63451, 63452, 63453, 63454, 63455, 63456) c6( ) c7(gx, T, nZ, Mb, aA, bB, n, ci, bh, aif, jz, aE, lt, Du, aie, jf, B, aig, ack, aq, iR, cm, bE, iu); #c1(ASPH) c2(NP_001158223) c3(aspartyl/asparaginyl beta-hydroxylase isoform g) c4( ) c5(63457, 63458, 63459, 63460, 63461, 63462) c6( ) c7(gx, T, nZ, Mb, aA, bB, n, ci, bh, aif, jz, aE, lt, Ou, aie, jf, B, aig, ack, aq, iR, cm, bE, iu); #c1(ASPH) c2(NP_001158225) c3(aspartyl/asparaginyl beta-hydroxylase isoform i) c4( ) c5(63463, 63464, 63465, 63466, 63467, 63468) c6( ) c7(gx, T, nZ, Mb, aA, bB, n, ci, bh, aif, jz, aE, lt, Ou, aie, jf, B, aig, ack, aq, iR, cm, bE, iu); #c1(ASPH) c2(NP_001158227) c3(aspartyl/asparaginyl beta-hydroxylase isoform k) c4( ) c5(63469, 63470, 63471, 63472, 63473, 63474) c6( ) c7(gx, T, nZ, Mb, aA, bB, n, ci, bh, aif, jz, aE, lt, Ou, aie, jf, B, aig, ack, aq, iR, cm, bE, iu); #c1(ASPH) c2(NP_001158228) c3(aspartyl/asparaginyl beta-hydroxylase isoform I) c4(63475) c5(63476, 63477, 63478, 63479, 63480, 63481) c6( ) c7(gx, T, nZ, Mb, aA, bB, n, ci, bh, aif, jz, aE, lt, Ou, aie, jf, B, aig, ack, aq, iR, cm, bE, iu); #c1(ASPH) c2(NP_004309) c3(aspartyl/asparaginyl beta-hydroxylase isoform a) c4(145066, 145067) c5(145068, 145069, 145070, 145071, 145072, 145073) c6( ) c7(gx, T, nZ, Mb, aA, bB, n, ci, bh, aif, jz, aE, lt, Ou, aie, jf, B, aig, ack, aq, iR, cm, bE, iu); #c1(ASPH) c2(NP_064549) c3(aspartyl/asparaginyl beta-hydroxylase isoform e) c4(202937) c5(202938, 202939, 202940, 202941, 202942, 202943) c6( ) c7(gx, T, nZ, Mb, aA, bB, n, ci, bh, aif, jz, aE, lt, Ou, aie, jf, B, aig, ack, aq, iR, cm, bE, iu); #c1(ASPH) c2(NP_115855) c3(aspartyl/asparaginyl beta-hydroxylase isoform c) c4( ) c5(224315, 224316, 224317, 224318, 224319, 224320) c6( ) c7(gx, T, nZ, Mb, aA, bB, n, ci, bh, aif, jz, aE, lt, Ou, aie, jf, B, aig, ack, aq, iR, cm, bE, iu); #c1(ASPH) c2(NP_115856) c3(aspartyl/asparaginyl beta-hydroxylase isoform d) c4(224321) c5(224322, 224323, 224324, 224325, 224326, 224327) c6( ) c7(gx, T, nZ, Mb, aA, bB, n, ci, bh, aif, jz, aE, lt, Ou, aie, jf, B, aig, ack, aq, iR, cm, bE, iu); #c1(ASPH) c2(NP_115857) c3(aspartyl/asparaginyl beta-hydroxylase isoform b) c4( ) c5(224328, 224329, 224330, 224331, 224332, 224333, 224334, 224335, 224336, 224337) c6( ) c7(gx, T, nZ, Mb, aA, bB, n, ci, bh, aif, jz, aE, lt, Ou, aie, jf, B, aig, ack, aq, iR, cm, bE, iu); #c1(ASPH) c2(XP_005251297) c3 (aspartyl/asparaginyl beta-hydroxylase isoform X6) c4(270871) c5( ) c6( ) c7(gx, T, nZ, Mb, aA, bB, n, ci, bh, aif, jz, aE, lt, Ou, aie, jf, B, aig, ack, aq, iR, cm, bE, iu); #c1(ASPH) c2(XP_005251298) c3(aspartyl/asparaginyl beta-hydroxylase isoform X7) c4(270872) c5( ) c6( ) c7(gx, T, nZ, Mb, aA, bB, n, ci, bh, aif, jz, aE, lt, Ou, aie, jf, B, aig, ack, aq, iR, cm, bE, iu); #c1(ASPH) c2(XP_005251299) c3 (aspartyl/asparaginyl beta-hydroxylase isoform X8) c4(270873) c5(270874, 270875, 270876, 270877, 270878, 270879) c6( ) c7(gx, T, nZ, Mb, aA, bB, n, ci, bh, aif, jz, aE, lt, Ou, aie, jf, B, aig, ack, aq, iR, cm, bE, iu); #c1(ASPH) c2(XP_005251303) c3 (aspartyl/asparaginyl beta-hydroxylase isoform X11) c4(270880) c5( ) c6( ) c7(gx, T, nZ, Mb, aA, bB, n, ci, bh, aif, jz, aE, lt, Ou, aie, jf, B, aig, ack, aq, iR, cm, bE, iu); #c1(ASPH) c2(XP_005251306) c3(aspartyl/asparaginyl beta-hydroxylase isoform X14) c4(270881) c5( ) c6( ) c7(gx, T, nZ, Mb, aA, bB, n, ci, bh, aif, jz, aE, lt, Ou, aie, jf, B, aig, ack, aq, iR, cm, bE, iu); #c1(ASPM) c2(NP_001193775) c3(abnormal spindle-like microcephaly-associated protein isoform 2) c4(82586, 82587) c5(82588, 82589, 82590, 82591, 82592, 82593, 82594, 82595, 82596, 82597, 82598, 82599) c6( ) c7(T, aC, p, n, v, j, nA, eA, o, x, l, u, HJ, qK, aT, h, ey, aij, aii, d, kl, A, aih, kp, QK, t, kP, c, W, w, FB, agl, m, k, y, B, ahZ); #c1(ASPM) c2(NP_060606) c3(abnormal spindle-like microcephaly-associated protein isoform 1) c4(196808, 196809) c5(196810, 196811, 196812, 196813, 196814, 196815, 196816, 196817, 196818, 196819, 196820, 196821, 196822, 196823, 196824, 196825, 196826) c6( ) c7(T, aC, p, n, v, j, nA, eA, o, x, l, u, HJ, qK, aT, h, ey, aij, aii, d, kl, A, aih, kp, QK, t, kP, c, W, w, FB, agl, m, k, y, B, ahZ); #c1(ASPN) c2(NP_001180264) c3(asporin isoform 2 preproprotein) c4(73582, 73583) c5(73584, 73585, 73586, 73587, 73588, 73589, 73590, 73591, 73592, 73593, 73594, 73595, 73596, 73597, 73598, 73599, 73600, 73601) c6( ) c7(aik, mR, rw, ail, rE, y, rp); #c1(ASPN) c2(NP_060150) c3(asporin isoform 1 preproprotein) c4(194504, 194505) c5(194506, 194507, 194508, 194509, 194510, 194511) c6( ) c7(aik, mR, rw, ail, rE, y, rp); #c1(ASPRV1) c2(NP_690005) c3(retroviral-like aspartic protease 1) c4(244711) c5(244712, 244713, 244714, 244715, 244716, 244717) c6( ) c7(aW, ff, aT, K, en, eF); #c1(ASPSCR1) c2(NP_001238817) c3(tether containing UBX domain for GLUT4 isoform 2) c4(87935) c5(87936, 87937, 87938, 87939, 87940, 87941) c6( ) c7(bL, es, T, QG, fr, ci, g, Vi, aim); #c1(ASPSCR1) c2(NP_076988) c3(tether containing UBX domain for GLUT4 isoform 1) c4(214635, 214636) c5(214637, 214638, 214639, 214640, 214641, 214642, 214643, 214644, 214645, 214646, 214647, 214648) c6( ) c7(bL, es, T, QG, fr, ci, g, Vi, aim); #c1(ASRGL1) c2(NP_001077395) c3(isoaspartyl peptidase/L-asparaginase) c4(37816, 37817) c5(37818, 37819, 37820, 37821, 37822, 37823, 37824, 37825, 37826, 37827) c6( ) c7(T, ul, aio, jh, fd, bB, aeu, dG, nA, ain, x, TI, aE, aT, h, dQ, nJ, bc, afy, vT, HY, aW, TH, fJ, eH, aq, eF, t, ck, iT, TJ, bE, TG, au, rx, m, k, y, bt, B); #c1(ASRGL1) c2(XP_005274363) c3(isoaspartyl peptidase/L-asparaginase isoform X5) c4( ) c5(285061, 285062, 285063, 285064, 285065) c6( ) c7(T, ul, aio, jh, fd, bB, aeu, dG, nA, ain, x, TI, aE, aT, h, dQ, nJ, bc, afy, vT, HY, aW, TH, fJ, eH, aq, eF, t, ck, iT, TJ, bE, TG, au, rx, m, k, y, bt, B); #c1(ASRGL1) c2(XP_011543566) c3(isoaspartyl peptidase/L-asparaginase isoform X1) c4( ) c5(342485) c6( ) c7(T, ul, aio, jh, fd, bB, aeu, dG, nA, ain, x, Tl, aE, aT, h, dQ, nJ, bc, afy, vT, HY, aW, TH, fJ, eH, aq, eF, t, ck, iT, TJ, bE, TG, au, rx, m, k, y, bt, B); #c1(ASS1) c2(XP_005272257) c3(argininosuccinate synthase isoform X1) c4(283794, 283795, 283796, 283797, 283798, 283799) c5(283800, 283801, 283802, 283803, 283804, 283805, 283806, 283807, 283808, 283809, 283810, 283811, 283812, 283813, 283814, 283815, 283816, 283817, 283818, 283819, 283820, 283821, 283822, 283823, 283824, 283825, 283826, 283827, 283828, 283829, 283830, 283831, 283832, 283833, 283834, 283835, 283836, 283837, 283838, 283839, 283840, 283841, 283842, 283843, 283844, 283845, 283846, 283847, 283848, 283849, 283850, 283851, 283852, 283853, 283854, 283855, 283856, 283857, 283858, 283859, 283860, 283861, 283862, 283863, 283864, 283865, 283866, 283867, 283868, 283869, 283870, 283871, 283872, 283873, 283874, 283875, 283876, 283877, 283878, 283879, 283880, 283881, 283882, 283883, 283884, 283885, 283886, 283887, 283888, 283889, 283890, 283891, 283892, 283893, 283894, 283895, 283896, 283897, 283898, 283899, 283900, 283901, 283902, 283903, 283904, 283905, 283906, 283907, 283908, 283909, 283910, 283911, 283912, 283913, 283914, 283915, 283916, 283917, 283918, 283919, 283920) c6(283921) c7(oO, ahW, T, Ve, jh, aip, aA, n, fr, C, pn, qW, ai, ad, Na, ft, aE, hb, aT, Lv, Nc, dl, jo, bq, ais, Wm, ae, eL, ux, jf, d, iU, nO, aW, Y, aiq, eH, eZ, fQ, aq, aF, eF, t, dr, ck, mO, RV, oh, m, air, bt, B, hS); #c1(ASS1) c2(XP_011517007) c3(argininosuccinate synthase isoform X2) c4(310492) c5(310493, 310494, 310495, 310496, 310497, 310498) c6( ) c7(oO, ahW, T, Ve, jh, aip, aA, n, fr, C, pn, qW, ai, ad, Na, ft, aE, hb, aT, Lv, Nc, dL, jo, bq, ais, Wm, ae, eL, uX, jf, d, iU, nO, aW, Y, aiq, eH, eZ, fQ, aq, aF, eF, t, dr, ck, mO, RV, oh, m, air, bt, B, hS); #c1(ASTN2) c2(NP_001171663) c3(astrotactin-2 isoform e) c4( ) c5(71229, 71230, 71231, 71232, 71233, 71234) c6( ) c7(ac, rz, cz, bK, j, vo, fq, xa, eu, bN); #c1(ASTN2) c2(NP_001171664) c3(astrotactin-2 isoform f) c4(71235) c5(71236, 71237, 71238, 71239, 71240, 71241, 71242) c6( ) c7(ac, rz, cz, bK, j, vo, fq, xa, eu, bN); #c1(ASTN2) c2(NP_054729) c3(astrotactin-2 isoform a precursor) c4(177394, 177395, 177396) c5(177397, 177398, 177399, 177400, 177401, 177402, 177403, 177404, 177405, 177406, 177407, 177408, 177409, 177410, 177411, 177412, 177413, 177414, 177415) c6( ) c7(ac, rz, cz, bK, j, vo, fq, xa, eu, bN); #c1(ASTN2) c2(NP_937829) c3(astrotactin-2 isoform b) c4(259235, 259236) c5(259237, 259238, 259239, 259240, 259241, 259242, 259243) c6( ) c7(ac, rz, cz, bk, j, vo, fq, xa, eu, bN); #c1(ASTN2) c2(NP_937831) c3(astrotactin-2 isoform d) c4(259244) c5(259245, 259246, 259247, 259248, 259249, 259250, 259251, 259252) c6( ) c7(ac, rz, cz, bK, j, vo, fq, xa, eu, bN); #c1(ASUN) c2(NP_060634) c3(protein asunder homolog) c4(196926, 196927) c5(196928, 196929, 196930, 196931, 196932, 196933, 196934, 196935, 196936, 196937, 196938, 196939, 196940) c6( ) c7(wu, PI, BJ, NI, wm, KY); #c1(ASUN) c2(XP_005253479) c3(protein asunder homolog isoform X3) c4( ) c5(272529, 272530, 272531, 272532, 272533) c6( ) c7(wu, PI, BJ, NI, wm, KY); #c1(ASUN) c2(XP_011519053) c3(protein asunder homolog isoform X2) c4(313556, 313557, 313558, 313559) c5(313560) c6( ) c7(wu, PI, BJ, NI, wm, KY); #c1(ASUN) c2(XP_011519054) c3(protein asunder homolog isoform X4) c4(313561) c5(313562, 313563, 313564, 313565, 313566, 313567, 313568, 313569, 313570, 313571, 313572, 313573, 313574, 313575, 313576, 313577, 313578, 313579) c6( ) c7(wu, PI, BJ, NI, wm, KY); #c1(ASUN) c2(XP_011519055) c3 (protein asunder homolog isoform X5) c4( ) c5(313580, 313581, 313582, 313583, 313584) c6( ) c7(wu, PI, BJ, NI, wm, KY); #c1(ASXL1) c2(NP_001158075) c3(putative Polycomb group protein ASXL1 isoform 2) c4(63083) c5(63084, 63085, 63086, 63087, 63088, 63089, 63090, 63091, 63092, 63093, 63094, 63095, 63096, 63097, 63098, 63099, 63100) c6( ) c7(gV, aiu, T, Js, if, qx, qy, fZ, bs, n, ol, aeu, oY, fd, qN, jh, abd, hF, br, ad, x, OB, oT, oW, aT, aU, vw, dz, h, j, pq, ae, Nr, aiw, oP, iU, aix, hG, aW, Y, et, gK, pr, r, Mz, kD, po, gm, pm, bw, aiv, bz, yR, Nq, yd, ck, Ta, pc, UY, ait, iT, kz, cj, pg, dW, k, y, bt, Jo, Tc); #c1(ASXL1) c2(NP_056153) c3(putative Polycomb group protein ASXL1 isoform 1) c4(185987, 185988) c5(185989, 185990, 185991, 185992, 185993, 185994, 185995, 185996, 185997, 185998, 185999, 186000, 186001, 186002) c6( ) c7(gV, aiu, T, Js, if, qx, qy, fZ, bs, n, ol, aeu, oY, fd, qN, jh, abQ, hF, br, ad, x, OB, oT, oW, aT, aU, vw, dz, h, j, pq, ae, Nr, aiw, oP, iU, aix, hG, aW, Y, et, gK, pr, r, Mz, kD, po, gm, pm, bw, aiv, bz, yR, Nq, yd, ck, Ta, pc, UY, ait, iT, kz, cj, pg, dW, k, y, bt, Jo, Tc); #c1(ASXL1) c2(XP_006723790) c3 (putative Polycomb group protein ASXL1 isoform X5) c4(295398, 295399) c5(295400) c6( ) c7(gV, aiu, T, Js, if, qx, qy, fZ, bs, n, ol, aeu, oY, fd, qN, jh, abQ, hF, br, ad, x, oB, oT, oW, aT, aU, vw, dz, h, jj, pq, ae, Nr, aiw, oP, iU, aix, hG, aW, Y, et, gK, pr, r, Mz, kD, po, gm, pm, bw, aiv, bz, yR, Nq, yd, ck, Ta, pc, UY, ait, iT, kz, cj, pg, dW, k, y, bt, Jo, Tc); #c1(ASXL1) c2(XP_006723795) c3(putative Polycomb group protein ASXL1 isoform X9) c4(295401) c5(295402, 295403, 295404, 295405, 295406) c6( ) c7(gV, aiu, T, Js, if, qx, qy, fZ, bs, n, ol, aeu, oY, fd, qN, jh, abd, hF, br, ad, x, oB, oT, oW, aT, aU, vw, dz, h, j, pq, ae, Nr, aiw, oP, iU, aix, hG, aW, Y, et, gK, pr, r, Mz, kD, po, gm, pm, bw, aiv, bz, yR, Nq, yd, ck, Ta, pc, UY, ait, iT, kz, cj, pg, dW, k, y, bt, Jo, Tc); #c1(ASXL1) c2(XP_011526950) c3(putative Polycomb group protein ASXL1 isoform X2) c4( ) c5(321238, 321239, 321240, 321241, 321242, 321243) c6( ) c7(gV, aiu, T, Js, if, qx, qy, fZ, bs, n, ol, aeu, oY, fd, qN, jh, abd, hF, br, ad, x, oB, oT, oW, aT, aU, vw, dz, h, j, pq, ae, Nr, aiw, oP, iU, aix, hG, aW, Y, et, gK, pr, r, Mz, kD, po, gm, pm, bw, aiv, bz, yR, Nq, yd, ck, Ta, pc, UY, ait, iT, kz, cj, pg, dW, k, y, bt, Jo, Tc); #c1(ASXL1) c2(XP_011526952) c3(putative Polycomb group protein ASXL1 isoform X4) c4( ) c5(321244) c6( ) c7(gV, aiu, T, Js, if, qx, qy, fZ, bs, n, ol, aeu, oY, fd, qN, jh, abQ, hF, br, ad, x, OB, oT, oW, aT, aU, vw, dz, h, j, pq, ae, Nr, aiw, oP, iU, aix, hG, aW, Y, et, gK, pr, r, Mz, kD, po, gm, pm, bw, aiv, bz, yR, Nq, yd, ck, Ta, pc, UY, ait, iT, kz, cj, pg, dW, k, y, bt, Jo, Tc); #c1(ASXL2) c2(NP_060733) c3(putative Polycomb group protein ASXL2) c4(197514, 197515) c5(197516, 197517, 197518, 197519, 197520, 197521, 197522, 197523, 197524, 197525, 197526, 197527, 197528, 197529, 197530, 197531, 197532, 197533, 197534, 197535, 197536, 197537) c6( ) c7(iM, je, Y, aT, gZ, r, dL, mo, ae, ad); #c1(ASXL2) c2(XP_006712103) c3(putative Polycomb group protein ASXL2 isoform X4) c4( ) c5(286401) c6( ) c7(iM, je, Y, aT, gZ, r, dL, mo, ae, ad); #c1(ASXL2) c2(XP_011531253) c3 (putative Polycomb group protein ASXL2 isoform X2) c4(327231) c5( ) c6( ) c7(iM, je, Y, aT, gZ, r, d, mo, ae, ad); #c1(ASXL3) c2(NP_085135) c3(putative Polycomb group protein ASXL3) c4(219395) c5(219396, 219397, 219398, 219399, 219400, 219401) c6( ) c7(aiy, aW, Y, Az, r, aT, cm, QK); #c1(ASZ1) c2(NP_570124) c3(ankyrin repeat, SAM and basic leucine zipper domain-containing protein 1 isoform 1) c4(233983) c5(233984, 233985, 233986, 233987, 233988, 233989) c6( ) c7(dA, DK, e, Nf, lo, j, BF, ad, vi); #c1(ATAD2) c2(NP_054828) c3(ATPase family AAA domain-containing protein 2) c4(178103) c5(178104, 178105, 178106, 178107, 178108, 178109) c6( ) c7(iu, T, eW, MD, aW, aT, h, n, aq, ey, qF, cl, iR, ae, B, ad); #c1(ATAD3B) c2(XP_011540545) c3(ATPase family AAA domain-containing protein 38 isoform X3) c4( ) c5(339040, 339041, 339042, 339043, 339044, 339045, 339046) c6( ) c7(aW, T, Fc, as, aT); #c1(ATAD3B) c2(XP_011540546) c3(ATPase family AAA domain-containing protein 3B isoform X5) c4(339047) c5(339048, 339049) c6( ) c7(aW, T, Fc, as, aT); #c1(ATAD3C) c2(XP_011539310) c3(ATPase family AAA domain-containing protein 3C isoform X2) c4( ) c5(337370) c6( ) c7(iu, d); #c1(ATAD3C) c2(XP_011539311) c3(ATPase family AAA domain-containing protein 3C isoform X1) c4(337371) c5(337372, 337373, 337374, 337375, 337376, 337377, 337378, 337379, 337380, 337381) c6( ) c7(iu, d); #c1(ATAD5) c2(NP_079133) c3(ATPase family AAA domain-containing protein 5) c4(217201) c5(217202, 217203, 217204, 217205, 217206, 217207) c6( ) c7(T, os, nX, eH, Az, ml, oZ, aiz, hP, K, g, eF, xb, dr, xt); #c1(ATAT1) c2(NP_001026892) c3(alpha-tubulin N-acetyltransferase 1 isoform 1 precursor) c4(29891) c5(29892, 29893, 29894, 29895, 29896, 29897, 29898, 29899, 29900, 29901, 29902, 29903, 29904, 29905, 29906, 29907, 29908, 29909, 29910, 29911, 29912, 29913, 29914, 29915) c6( ) c7(bL, T, DM, fM, Ke, e, DV, jh, aiC, bS, gf, ct, C, aiA, fr, ai, ad, eA, eJ, ip, x, eW, aE, pX, aT, h, cy, K, ae, pi, mu, le, aiB, qt, aW, oV, CM, cr, eZ, yq, gu, as, Z, kk, cm, ek, ck, pH, ok, iQ, c, eM, bE, Uq, U, QF, aiD, mf, m, k, EY, be, ym, bt, kS, y); #c1(ATAT1) c2(NP_001241881) c3 (alpha-tubulin N-acetyltransferase 1 isoform 4) c4(89149) c5(89150, 89151, 89152, 89153, 89154, 89155, 89156, 89157, 89158, 89159, 89160, 89161, 89162, 89163, 89164, 89165, 89166, 89167, 89168, 89169, 89170, 89171, 89172, 89173, 89174, 89175, 89176, 89177, 89178, 89179, 89180, 89181, 89182, 89183, 89184, 89185, 89186, 89187, 89188, 89189, 89190, 89191, 89192, 89193, 89194, 89195, 89196, 89197, 89198, 89199, 89200, 89201) c6( ) c7(bL, T, DM, fM, Ke, e, DV, jh, aiC, bS, gf, ct, C, aiA, fr, ai, ad, eA, eJ, ip, x, eW, aE, pX, aT, h, cy, K, ae, pi, mu, le, aiB, qt, aW, oV, CM, cr, eZ, yq, gu, as, Z, kk, cm, ek, ck, pH, ok, iQ, c, eM, bE, Uq, U, QF, aiD, mf, m, k, EY, be, ym, bt, kS, y); #c1(ATAT1) c2(NP_079185) c3(alpha-tubulin N-acetyltransferase 1 isoform 2) c4(217348) c5(217349, 217350, 217351, 217352, 217353, 217354, 217355, 217356, 217357, 217358, 217359, 217360, 217361, 217362, 217363, 217364, 217365, 217366, 217367, 217368, 217369, 217370, 217371, 217372, 217373, 217374, 217375, 217376, 217377, 217378, 217379, 217380, 217381, 217382, 217383, 217384, 217385, 217386, 217387, 217388, 217389, 217390, 217391, 217392, 217393, 217394, 217395, 217396, 217397, 217398, 217399, 217400) c6( ) c7(bL, T, DM, fM, Ke, e, DV, jh, aiC, bS, gf, ct, C, aiA, fr, ai, ad, eA, eJ, ip, x, eW, aE, pX, aT, h, cy, K, ae, pi, mu, le, aiB, qt, aW, oV, CM, cr, eZ, yq, gu, as, Z, kk, cm, ek, ck, pH, ok, iQ, c, eM, bE, Uq, U, QF, aiD, mf, m, k, EY, be, ym, bt, kS, y); #c1(ATAT1) c2(XP_005249473) c3(alpha-tubulin N-acetyltransferase 1 isoform X3) c4(269445) c5( ) c6( ) c7(bL, T, DM, fM, Ke, e, DV, jh, aiC, bS, gf, ct, C, aiA, fr, ai, ad, eA, eJ, ip, x, eW, aE, pX, aT, h, cy, K, ae, pi, mu, le, aiB, qt, aW, oV, CM, cr, eZ, yq, gu, as, Z, kk, cm, ek, ck, pH, ok, iQ, c, eM, bE, Uq, U, QF, aiD, mf, m, k, EY, be, ym, bt, kS, y); #c1(ATAT1) c2(XP_005249474) c3(alpha-tubulin N-acetyltransferase 1 isoform X4) c4(269446, 269447, 269448) c5(269449, 269450, 269451, 269452, 269453, 269454, 269455, 269456, 269457, 269458, 269459, 269460, 269461, 269462, 269463, 269464, 269465, 269466, 269467, 269468, 269469, 269470, 269471, 269472, 269473, 269474, 269475, 269476, 269477, 269478, 269479, 269480, 269481, 269482, 269483, 269484, 269485, 269486, 269487, 269488, 269489, 269490, 269491, 269492, 269493, 269494, 269495, 269496) c6( ) c7(bL, T, DM, fM, Ke, e, DV, jh, aiC, bS, gf, ct, C, aiA, fr, ai, ad, eA, eJ, ip, x, eW, aE, pX, aT, h, cy, K, ae, pi, mu, le, aiB, qt, aW, oV, CM, cr, eZ, yq, gu, as, Z, kk, cm, ek, ck, pH, ok, iQ, c, eM, bE, Uq, U, QF, aiD, mf, m, k, EY, be, ym, bt, kS, y); #c1(ATCAY) c2(NP_149053) c3(caytaxin) c4(226933, 226934, 226935, 226936, 226937) c5(226938, 226939, 226940, 226941, 226942, 226943, 226944, 226945, 226946, 226947, 226948, 226949, 226950, 226951, 226952, 226953, 226954, 226955, 226956, 226957, 226958, 226959, 226960, 226961, 226962, 226963, 226964, 226965, 226966, 226967) c6( ) c7(Ng, ky, lF, dr, aU, EH, he, ci, aiE, nA, H, aR, L); #c1(ATE1) c2(NP_001001976) c3(arginyl-tRNA-protein transferase 1 isoform 1) c4(19264, 19265) c5(19266, 19267, 19268, 19269, 19270, 19271, 19272, 19273, 19274, 19275, 19276, 19277, 19278) c6( ) c7(cd); #c1(ATE1) c2(NP_001275663) c3(arginyl-tRNA --protein transferase 1 isoform 3) c4(106961, 106962) c5(106963, 106964, 106965, 106966, 106967, 106968, 106969, 106970, 106971, 106972, 106973) c6( ) c7(cd); #c1(ATE1) c2(NP_001275664) c3(arginyl-tRNA-protein transferase 1 isoform 4) c4(106974) c5(106975, 106976, 106977, 106978, 106979) c6( ) c7(cd); #c1(ATE1) c2(NP_001275665) c3(arginyl-tRNA-protein transferase 1 isoform 5) c4(106980) c5(106981, 106982, 106983, 106984, 106985, 106986, 106987, 106988, 106989, 106990, 106991, 106992, 106993) c6( ) c7(cd); #c1(ATE1) c2(NP_008972) c3(arginyl-tRNA-protein transferase 1 isoform 2) c4(169980) c5(169981, 169982, 169983, 169984, 169985, 169986) c6( ) c7(cd); #c1(ATE1) c2(XP_005269516) c3 (arginyl-tRNA-protein transferase 1 isoform X2) c4(282612) c5( ) c6( ) c7(cd); #c1(ATF1) c2(XP_011536689) c3(cyclic AMP-dependent transcription factor ATF-1 isoform X1) c4(334196, 334197, 334198, 334199) c5(334200, 334201, 334202, 334203, 334204, 334205, 334206, 334207, 334208, 334209, 334210, 334211, 334212, 334213, 334214, 334215, 334216, 334217, 334218) c6( ) c7(T, aiF, aiH, ci, lu, eA, nv, aT, K, dx, cp, kl, d, Y, hQ, mo, adj, t, QA, nM, Dy, wB, gX, aiG, bt); #c1(ATF1) c2(XP_011536690) c3(cyclic AMP-dependent transcription factor ATF-1 isoform X2) c4( ) c5(334219, 334220, 334221, 334222, 334223, 334224) c6( ) c7(T, aif, aiH, ci, lu, eA, nv, aT, K, dx, cp, kl, d, Y, hl, mo, adj, t, [A, nM, Dy, wB, gX, aiG, bt); #c1(ATF2) c2(NP_001243019) c3(cyclic AMP-dependent transcription factor ATF-2 isoform 1) c4(89541, 89542, 89543, 89544, 89545, 89546) c5(89547, 89548, 89549, 89550, 89551, 89552, 89553, 89554, 89555, 89556, 89557, 89558, 89559, 89560, 89561, 89562, 89563, 89564, 89565, 89566, 89567, 89568, 89569, 89570, 89571, 89572, 89573, 89574, 89575, 89576, 89577) c6( ) c7(ail, T, jh, bS, ct, iJ, ad, av, afQ, aT, K, jo, an, ae, af, cp, je, iE, aW, Y, ff, lo, en, hf, Z, cm, ik, bz, rR, iM, hk, U, Kg, y); #c1(ATF2) c2(NP_001243020) c3(cyclic AMP-dependent transcription factor ATF-2 isoform 2) c4(89578, 89579) c5(89580, 89581, 89582, 89583, 89584, 89585, 89586, 89587, 89588, 89589) c6( ) c7(ail, T, jh, bS, ct, iJ, ad, av, afQ, aT, K, jo, an, ae, af, cp, je, iE, aW, Y, ff, lo, en, hf, Z, cm, ik, bz, rR, iM, hk, U, Kg, y); #c1(ATF2) c2(NP_001243021) c3(cyclic AMP-dependent transcription factor ATF-2 isoform 3) c4(89590, 89591) c5(89592, 89593, 89594, 89595, 89596, 89597, 89598, 89599, 89600, 89601, 89602, 89603, 89604, 89605, 89606, 89607) c6( ) c7(ail, T, jh, bS, ct, iJ, ad, av, afQ, aT, K, jo, an, ae, af, cp, je, iE, aW, Y, ff, lo, en, hf, Z, cm, ik, bz, rR, iM, hk, U, Kg, y); #c1(ATF2) c2(NP_001243023) c3(cyclic AMP-dependent transcription factor ATF-2 isoform 5) c4( ) c5(89608, 89609, 89610, 89611, 89612, 89613, 89614, 89615, 89616, 89617, 89618, 89619, 89620, 89621, 89622, 89623, 89624) c6( ) c7(ail, T, jh, bS, ct, iJ, ad, av, afQ, aT, K, jo, an, ae, af, cp, je, iE, aW, Y, ff, lo, en, hf, Z, cm, ik, bz, rR, iM, hk, U, Kg, y); #c1(ATF3) c2(NP_001035709) c3(cyclic AMP-dependent transcription factor ATF-3 isoform 2) c4(32976) c5(32977, 32978, 32979, 32980, 32981, 32982, 32983, 32984, 32985, 32986) c6( ) c7(bL, bM, G, T, wi, aik, yk, if, bB, aj, dG, Sv, ci, C, bX, j, v, V, qN, q, aR, J, ad, er, fU, av, cc, aE, ds, kH, aT, lG, cy, dL, bK, K, bj, bq, he, KV, ae, eL, aiJ, JU, d, yi, iU, A, CH, aW, Y, je, ff, CT, r, fe, aq, Pp, zC, en, La, t, cm, bz, op, DF, iP, iM, bP, eM, bE, wl, wE, vA, wR, fD, ei, adg, hS); #c1(ATF3) c2(NP_001193413) c3(cyclic AMP-dependent transcription factor ATF-3 isoform 3) c4(82167) c5(82168, 82169, 82170, 82171, 82172) c6( ) c7(bL, bM, G, T, wi, aik, yk, if, bB, aj, dG, Sv, ci, C, bX, j, v, V, qN, q, aR, J, ad, er, fU, av, cc, aE, ds, kH, aT, lG, cy, dL, bK, K, bj, bq, he, KV, ae, eL, aiJ, JU, d, yi, iU, A, cH, aW, Y, je, ff, CT, r, fe, aq, Pp, zC, en, La, t, cm, bz, op, DF, iP, iM, bP, eM, bE, wl, wE, vA, wR, fD, ei, adg, hS); #c1(ATF3) c2(NP_001193415) c3(cyclic AMP-dependent transcription factor ATF-3 isoform 5) c4(82173) c5(82174, 82175, 82176, 82177, 82178, 82179) c6( ) c7(bL, bM, G, T, wi, aik, yk, if, bB, aj, dG, Sv, ci, C, bX, j, v, V, qN, q, aR, J, ad, er, fU, av, cc, aE, ds, kH, aT, lG, cy, dL, bK, K, bj, bq, he, KV, ae, eL, aiJ, JU, d, yi, iU, A, CH, aW, Y, je, ff, CT, r, fe, aq, Pp, zC, en, La, t, cm, bz, op, DF, iP, iM, bP, eM, bE, wl, wE, vA, wR, fD, ei, adg, hS); #c1(ATF3) c2(XP_011507881) c3(cyclic AMP-dependent transcription factor ATF-3 isoform X1) c4(298075, 298076) c5(298077, 298078, 298079, 298080, 298081, 298082, 298083, 298084, 298085, 298086, 298087, 298088, 298089, 298090, 298091, 298092, 298093, 298094) c6( ) c7(bL, bM, G, T, wi, aik, yk, if, bB, aj, dG, Sv, ci, C, bX, j, v, V, qN, q, aR, J, ad, er, fU, av, cc, aE, ds, kH, aT, lG, cy, dL, bK, K, bj, bq, he, KV, ae, eL, aiJ, JU, d, yi, iU, A, CH, aW, Y, je, ff, CT, r, fe, aq, Pp, zC, en, La, t, cm, bz, op, DF, iP, iM, bP, eM, bE, wO, wE, vA, wR, fD, ei, adg, hS); #c1(ATF4) c2(NP_001666) c3(cyclic AMP-dependent transcription factor ATF-4) c4(119840) c5(19841, 119842, 119843, 119844, 119845, 119846, 119847, 119848, 119849, 119850, 119851, 119852, 119853, 119854, 119855) c6( ) c7(ID, T, aA, q, aT, bK, K, he, dF, af, ly, lR, qt, aW, r, ail, aq, aF, cm, bz, lx, LB, eM, SE, at, B); #c1(ATF5) c2(NP_001277675) c3(cyclic AMP-dependent transcription factor ATF-5) c4(109387) c5(109388, 109389, 109390, 109391, 109392, 109393, 109394) c6( ) c7(ck, T, d, fR, aW, aT, bK, K, eA, pz, cm, B); #c1(ATF6) c2(NP_031374) c3(cyclic AMP-dependent transcription factor ATF-6 alpha) c4(172038) c5(172039, 172040, 172041, 172042, 172043, 172044, 172045) c6( ) c7(bL, lD, am, BD, e, jh, fd, aj, cd, ad, aT, cy, K, an, ae, ly, lR, aW, Y, fc, Mz, fe, cm, DF, lx, bN, c, Fi, aiM, xh, KS, B); #c1(ATF6B) c2(NP_001129625) c3(cyclic AMP-dependent transcription factor ATF-6 beta isoform b) c4(52023, 52024) c5(52025, 52026, 52027, 52028, 52029, 52030, 52031, 52032, 52033, 52034, 52035, 52036, 52037, 52038, 52039, 52040, 52041, 52042, 52043, 52044, 52045, 52046, 52047, 52048, 52049, 52050, 52051, 52052, 52053, 52054, 52055, 52056, 52057, 52058, 52059, 52060) c6( ) c7(eZ); #c1(ATF6B) c2(NP_004372) c3(cyclic AMP-dependent transcription factor ATF-6 beta isoform a) c4(145843, 145844) c5(145845, 145846, 145847, 145848, 145849, 145850, 145851, 145852, 145853, 145854, 145855, 145856, 145857, 145858, 145859, 145860, 145861, 145862, 145863, 145864, 145865, 145866, 145867, 145868, 145869, 145870, 145871, 145872, 145873, 145874, 145875, 145876) c6( ) c7(eZ); #c1(ATF7) c2(NP_001123532) c3(cyclic AMP-dependent transcription factor ATF-7 isoform 3) c4(47429) c5(47430, 47431, 47432, 47433, 47434, 47435, 47436, 47437, 47438, 47439, 47440, 47441) c6( ) c7(K, jh); #c1(ATF7) c2(NP_001193611) c3(cyclic AMP-dependent transcription factor ATF-7 isoform 4) c4(82355) c5(82356, 82357, 82358, 82359, 82360, 82361, 82362, 82363, 82364, 82365) c6( ) c7(K, jh); #c1(ATF7) c2(XP_005268644) c3(cyclic AMP-dependent transcription factor ATF-7 isoform X1) c4(282003) c5(282004, 282005, 282006, 282007, 282008, 282009, 282010, 282011, 282012, 282013) c6( ) c7(K, jh); #c1(ATF7) c2(XP_011536080) c3(cyclic AMP-dependent transcription factor ATF-7 isoform X2) c4(333393, 333394) c5(333395, 333396, 333397, 333398, 333399, 333400, 333401, 333402, 333403, 333404) c6( ) c7(K, jh); #c1(ATF7IP) c2(NP_001273444) c3(activating transcription factor 7-interacting protein 1 isoform 4) c4(104903, 104904, 104905, 104906, 104907, 104908, 104909, 104910) c5(104911, 104912, 104913, 104914, 104915, 104916) c6( ) c7(bL, T, aiR, jh, gf, bS, ct, mF, aR, aiN, rw, kH, Ne, h, rB, K, he, mH, ail, je, xR, Lf, Ce, lo, bu, Z, Ld, op, aiO, hq, ou, lt, iM, cA, aiP, vA, U, Lb, aiS, ph, zu, bt, ao); #c1(ATF7IP) c2(NP_851997) c3(activating transcription factor 7-interacting protein 1 isoform 1) c4(254756) c5(254757, 254758, 254759, 254760, 254761, 254762, 254763, 254764, 254765, 254766, 254767, 254768) c6( ) c7(bL, T, aiR, jh, gf, bS, ct, mF, aR, aiN, rw, kH, Ne, h, rB, K, he, mH, ail, je, xR, Lf, Ce, lo, bu, Z, Ld, op, aiO, hq, ou, lt, iM, cA, aiP, vA, U, Lb, aiS, ph, zu, bt, ao); #c1(ATF7IP) c2(XP_011519057) c3(activating transcription factor 7-interacting protein 1 isoform X1) c4(313585, 313586, 313587, 313588) c5(313589, 313590, 313591, 313592, 313593, 313594, 313595, 313596, 313597) c6( ) c7(bL, T, aiR, jh, gf, bS, ct, mF, aR, aiN, rw, kH, Ne, h, rB, K, he, mH, aiQ, je, xR, Lf, Ce, lo, bu, Z, Ld, op, aiO, hq, ou, lt, iM, cA, aiP, vA, U, Lb, aiS, ph, zu, bt, ao); #c1(ATF7IP) c2(XP_011519058) c3(activating transcription factor 7-interacting protein 1 isoform X3) c4(313598) c5( ) c6( ) c7(bL, T, aiR, jh, gf, bS, ct, mF, aR, aiN, rw, KH, Ne, h, rB, K, he, mH, ail, je, xR, Lf, Ce, lo, bu, Z, Ld, op, aiO, hq, ou, lt, iM, cA, aiP, vA, U, Lb, aiS, ph, zu, bt, ao); #c1(ATF7IP) c2(XP_011519060) c3(activating transcription factor 7-interacting protein 1 isoform X5) c4( ) c5(313599, 313600, 313601, 313602, 313603, 313604, 313605, 313606, 313607, 313608, 313609, 313610) c6( ) c7(bL, T, aiR, jh, gf, bS, ct, mF, aR, aiN, rw, kH, Ne, h, rB, K, he, mH, ail, je, xR, Lf, Ce, lo, bu, Z, Ld, op, aiO, hq, ou, lt, iM, cA, aiP, vA, U, Lb, aiS, ph, zu, bt, ao); #c1(ATF7IP) c2(XP_011519061) c3(activating transcription factor 7-interacting protein 1 isoform X6) c4( ) c5(313611, 313612, 313613, 313614, 313615, 313616, 313617, 313618, 313619, 313620, 313621, 313622, 313623, 313624, 313625, 313626, 313627, 313628, 313629, 313630, 313631, 313632, 313633, 313634, 313635, 313636, 313637, 313638, 313639, 313640, 313641, 313642, 313643, 313644, 313645, 313646, 313647, 313648, 313649, 313650, 313651, 313652, 313653, 313654, 313655, 313656, 313657, 313658, 313659, 313660, 313661, 313662, 313663) c6( ) c7(bL, T, aiR, jh, gf, bS, ct, mF, aR, aiN, rw, kH, Ne, h, rB, K, he, mH, ail, je, xR, Lf, Ce, lo, bu, Z, Ld, op, aiO, hq, ou, lt, iM, cA, aiP, vA, U, Lb, aiS, ph, zu, bt, ao); #c1(ATG10) c2(XP_005248667) c3(ubiquitin-like-conjugating enzyme ATG10 isoform X1) c4(268493, 268494) c5(268495, 268496, 268497, 268498, 268499, 268500, 268501, 268502, 268503, 268504, 268505, 268506, 268507, 268508, 268509, 268510) c6( ) c7(T, q, r); #c1(ATG12) c2(NP_001264712) c3(ubiquitin-like protein ATG12 isoform 2) c4(97661) c5(97662, 97663, 97664, 97665, 97666) c6( ) c7(aW, eZ, r, aT, aiT, h, ci, fL, mC); #c1(ATG12) c2(NP_004698) c3(ubiquitin-like protein ATG12 isoform 1) c4(148891, 148892) c5(148893, 148894, 148895, 148896, 148897, 148898, 148899, 148900, 148901, 148902, 148903, 148904, 148905) c6( ) c7(aW, eZ, r, aT, aiT, h, ci, fL, mC); #c1(ATG16L1) c2(NP_060444) c3(autophagy-related protein 16-1 isoform 2) c4(195969) c5(195970, 195971, 195972, 195973, 195974, 195975) c6( ) c7(aiU, T, rR, lk, ra, Ht, az, ju, ge, HG, n, lo, Ni, hp, K, fe, iV, ji, xb); #c1(ATG16L1) c2(NP_110430) c3 (autophagy-related protein 16-1 isoform 1) c4(220326) c5(220327, 220328, 220329, 220330, 220331, 220332) c6( ) c7(ail, T, rR, lk, ra, Ht, az, ju, ge, HG, n, lo, Ni, hp, K, fe, iV, ji, xb); #c1(ATG16L1) c2(NP_942593) c3(autophagy-related protein 16-1 isoform 3) c4(260576) c5(260577, 260578, 260579, 260580, 260581, 260582, 260583, 260584, 260585, 260586, 260587) c6( ) c7(aiU, T, rR, lk, ra, Ht, az, ju, ge, HG, n, lo, Ni, hp, K, fe, iV, ji, xb); #c1(ATG16L1) c2(XP_011547155) c3 (autophagy-related protein 16-1 isoform X1) c4(345883, 345884) c5(345885, 345886, 345887, 345888, 345889, 345890) c6( ) c7(aiU, T, rR, lk, ra, Ht, az, ju, ge, HG, n, lo, Ni, hp, K, fe, iV, ji, xb); #c1(ATG16L1) c2(XP_011547158) c3(autophagy-related protein 16-1 isoform X4) c4(345891) c5(345892, 345893, 345894, 345895, 345896, 345897, 345898) c6( ) c7(ail, T, rR, lk, ra, Ht, az, ju, ge, HG, n, lo, Ni, hp, K, fe, iV, ji, xb); #c1(ATG2B) c2(NP_060506) c3(autophagy-related protein 2 homolog B) c4(196288) c5(196289, 196290, 196291, 196292, 196293, 196294) c6( ) c7(r); #c1(ATG3) c2(NP_071933) c3(ubiquitin-like-conjugating enzyme ATG3 isoform 1) c4(212387) c5(212388, 212389, 212390, 212391, 212392, 212393) c6( ) c7(HG, NQ, bz, iu, rR); #c1(ATG4A) c2(NP_443168) c3(cysteine protease ATG4A isoform a) c4(229691, 229692) c5(229693, 229694, 229695, 229696, 229697, 229698, 229699, 229700, 229701, 229702, 229703, 229704) c6( ) c7(t, hy, m, ne, BN); #c1(ATG4A) c2(NP_840054) c3(cysteine protease ATG4A isoform b) c4(253584, 253585) c5(253586, 253587, 253588, 253589, 253590, 253591, 253592) c6( ) c7(t, hy, m, ne, BN); #c1(ATG4A) c2(XP_011529147) c3(cysteine protease ATG4A isoform X4) c4( ) c5(324493, 324494, 324495, 324496, 324497) c6( ) c7(t, hy, m, ne, BN); #c1(ATG4B) c2(NP_037457) c3(cysteine protease ATG4B isoform a) c4(175588, 175589, 175590, 175591) c5(175592, 175593, 175594, 175595, 175596, 175597, 175598, 175599, 175600, 175601, 175602, 175603, 175604, 175605, 175606, 175607, 175608, 175609, 175610, 175611) c6( ) c7(B, il); #c1(ATG4B) c2(NP_847896) c3(cysteine protease ATG4B isoform b) c4(253656) c5(253657, 253658, 253659, 253660, 253661, 253662, 253663, 253664, 253665) c6( ) c7(B, iU); #c1(ATG4B) c2(XP_005247050) c3(cysteine protease ATG4B isoform X6) c4(267599) c5(267600, 267601, 267602, 267603, 267604, 267605) c6( ) c7(B, iU); #c1(ATG4B) c2(XP_005247052) c3(cysteine protease ATG4B isoform X2) c4(267606, 267607, 267608, 267609) c5( ) c6( ) c7(B, iU); #c1(ATG4B) c2(XP_005247053) c3(cysteine protease ATG4B isoform X4) c4( ) c5(267610, 267611, 267612, 267613, 267614, 267615, 267616, 267617, 267618, 267619, 267620, 267621, 267622, 267623, 267624, 267625, 267626, 267627, 267628, 267629, 267630, 267631, 267632, 267633, 267634, 267635) c6( ) c7(B, il); #c1(ATG4C) c2(XP_011540615) c3(cysteine protease ATG4C isoform X2) c4(339115) c5(339116, 339117, 339118, 339119, 339120, 339121, 339122, 339123) c6(339124, 339125) c7(T, oe, fq); #c1(ATG4C) c2(XP_011540616) c3(cysteine protease ATG4C isoform X3) c4( ) c5(339126, 339127, 339128, 339129, 339130, 339131, 339132, 339133, 339134, 339135, 339136, 339137, 339138, 339139, 339140, 339141) c6(339142, 339143, 339144, 339145) c7(T, oe, fq); #c1(ATG5) c2(NP_001273035) c3(autophagy protein 5 isoform a) c4(104223) c5(104224, 104225, 104226, 104227, 104228, 104229, 104230, 104231, 104232, 104233) c6( ) c7(bM, T, n, v, j, ci, pM, V, eA, o, ad, x, l, hd, u, sN, ds, HG, h, K, ae, EG, A, mR, aiV, iU, oV, Y, eZ, r, aiT, kp, EZ, t, rR, Dy, c, w, wR, dy, k, m, y, B, p); #c1(ATG5) c2(NP_001273036) c3(autophagy protein 5 isoform b precursor) c4(104234) c5(104235, 104236, 104237, 104238, 104239, 104240, 104241) c6( ) c7(bM, T, n, v, j, ci, pM, V, eA, o, ad, x, l, hd, u, sN, ds, HG, h, K, ae, EG, A, mR, aiV, iU, oV, Y, eZ, r, aiT, kp, EZ, t, rR, Dy, c, w, wR, dy, k, m, y, B, p); #c1(ATG5) c2(NP_001273040) c3(autophagy protein 5 isoform d) c4(104254) c5(104255, 104256, 104257, 104258, 104259) c6( ) c7(bM, T, n, v, j, ci, pM, V, eA, o, ad, x, l, hd, u, sN, ds, HG, h, K, ae, EG, A, mR, aiV, iU, oV, Y, eZ, r, aiT, kp, EZ, t, rR, Dy, c, w, wR, dy, k, m, y, B, p); #c1(ATG7) c2(NP_001129503) c3(ubiquitin-like modifier-activating enzyme ATG7 isoform b) c4(51745) c5(51746, 51747, 51748, 51749, 51750, 51751, 51752, 51753, 51754, 51755, 51756, 51757) c6( ) c7(bM, T, pM, ad, aT, HG, aiW, K, jj, ae, ak, d, aW, eH, kp, g, eF, ik, bz, GB, rR, eM, wz, ei, KS, B); #c1(ATG7) c2(NP_001138384) c3(ubiquitin-like modifier-activating enzyme ATG7 isoform c) c4(56463, 56464) c5(56465, 56466, 56467, 56468, 56469, 56470, 56471, 56472, 56473, 56474, 56475, 56476, 56477, 56478, 56479, 56480, 56481, 56482, 56483, 56484, 56485, 56486, 56487, 56488, 56489, 56490, 56491, 56492, 56493, 56494) c6( ) c7(bM, T, pM, ad, aT, HG, aiW, K, j, ae, ak, d, aW, eH, kp, g, eF, ik, bz, GB, rR, eM, wz, ei, kS, B); #c1(ATG7) c2(NP_006386) c3(ubiquitin-like modifier-activating enzyme ATG7 isoform a) c4(164560) c5(164561, 164562, 164563, 164564, 164565, 164566) c6( ) c7(bM, T, pM, ad, aT, HG, aiW, K, jj, ae, ak, d, aW, eH, kp, g, eF, ik, bz, GB, rR, eM, wz, ei, KS, B); #c1(ATG7) c2(XP_011531587) c3 (ubiquitin-like modifier-activating enzyme ATG7 isoform X9) c4(327709, 327710, 327711, 327712) c5( ) c6( ) c7(bM, T, pM, ad, aT, HG, aiW, K, jj, ae, ak, d, aW, eH, kp, g, eF, iK, bz, GB, rR, eM, wz, ei, kS, B); #c1(ATG9A) c2(NP_001070666) c3(autophagy-related protein 9A) c4(34696, 34697, 34698, 34699, 34700, 34701, 34702, 34703, 34704) c5(34705, 34706, 34707, 34708, 34709, 34710, 34711, 34712, 34713, 34714, 34715, 34716, 34717, 34718, 34719, 34720, 34721, 34722, 34723, 34724, 34725, 34726, 34727, 34728, 34729, 34730, 34731, 34732, 34733, 34734, 34735, 34736, 34737, 34738, 34739, 34740, 34741, 34742, 34743, 34744, 34745, 34746, 34747, 34748, 34749, 34750, 34751, 34752, 34753, 34754, 34755, 34756, 34757, 34758, 34759, 34760, 34761, 34762, 34763, 34764, 34765, 34766) c6( ) c7(BN, aiV, hy, wR, m, t, ne); #c1(ATIC) c2(NP_004035) c3(bifunctional purine biosynthesis protein PURH) c4(143004, 143005) c5(143006, 143007, 143008, 143009, 143010, 143011, 143012, 143013, 143014, 143015, 143016, 143017) c6( ) c7(JY, if, iW, jh, fd, cl, bS, v, ct, qN, fr, o, x, K, bA, nO, fO, cr, r, Z, ck, aix, lp, k, y, B); #c1(ATL1) c2(NP_001121185) c3(atlastin-1 isoform b) c4(45698, 45699, 45700) c5(45701, 45702, 45703, 45704, 45705, 45706, 45707, 45708, 45709, 45710, 45711, 45712, 45713, 45714, 45715, 45716, 45717) c6( ) c7(aiZ, aiY, Pt, OR, fu, aje, aV, lu, aja, afu, je, ajb, xw, fq, Qn, LB, iM, ac, c, ajc, ajd, U, bk, dy); #c1(ATL1) c2(NP_056999) c3(atlastin-1 isoform a) c4(188390, 188391) c5(188392, 188393, 188394, 188395, 188396, 188397, 188398, 188399, 188400, 188401, 188402, 188403, 188404, 188405, 188406, 188407, 188408) c6( ) c7(aiZ, aiY, Pt, oR, fu, aje, aV, lu, aja, afu, je, ajb, xw, fq, Qn, LB, iM, ac, c, ajc, ajd, U, bk, dy); #c1(ATL2) c2(NP_001129145) c3(atlastin-2 isoform 2) c4(50910) c5(50911, 50912, 50913, 50914, 50915, 50916, 50917, 50918) c6( ) c7(je, iM, kH); #c1(ATL2) c2(NP_071769) c3(atlastin-2 isoform 1) c4(212022) c5(212023, 212024, 212025, 212026, 212027, 212028) c6( ) c7(je, iM, kH); #c1(ATL2) c2(XP_006712135) c3(atlastin-2 isoform X1) c4(286425) c5( ) c6( ) c7(je, iM, kH); #c1(ATL2) c2(XP_011531321) c3(atlastin-2 isoform X4) c4(327313) c5(327314, 327315, 327316, 327317, 327318, 327319) c6( ) c7(je, iM, KH); #c1(ATL2) c2(XP_011531323) c3 (atlastin-2 isoform X6) c4( ) c5(327320) c6( ) c7(je, iM, kH); #c1(ATL2) c2(XP_011531324) c3(atlastin-2 isoform X7) c4(327321) c5(327322, 327323, 327324, 327325, 327326) c6( ) c7(je, iM, kH); #c1(ATL3) c2(NP_001276977) c3(atlastin-3 isoform 2) c4(108889) c5( ) c6( ) c7(ajf, lH, Pt); #c1(ATL3) c2(NP_056274) c3(atlastin-3 isoform 1) c4(186856) c5(186857, 186858, 186859, 186860, 186861, 186862, 186863, 186864, 186865, 186866, 186867, 186868) c6( ) c7(ajf, lH, Pt); #c1(ATM) c2(XP_006718906) c3(serine-protein kinase ATM isoform X1) c4(292060) c5(292061, 292062, 292063, 292064, 292065, 292066, 292067, 292068, 292069, 292070, 292071, 292072, 292073, 292074, 292075, 292076, 292077, 292078, 292079, 292080) c6( ) c7(bM, T, os, aC, fR, ea, if, e, al, hE, fd, ajp, bB, lR, ci, Lw, aY, aR, ip, x, bv, aji, aE, pX, ajv, M, aU, iv, K, hu, eL, bc, N, tw, zQ, iE, aW, WE, hl, adp, g, as, pl, eF, cm, bw, aj, qJ, y, eM, nB, iT, U, cj, k, m, pw, jM, iu, Ro, FW, dD, ajm, iB, aw, ht, ajg, n, cQ, aj, ct, qN, cd, br, ai, eW, b, ft, wG, i, gd, kH, h, eV, hn, dl, on, pl, Ck, OV, OS, RT, A, aq, kp, Hu, ajo, QK, OU, kT, W, ky, dB, hk, ajn, hL, MM, at, oh, WL, cB, B, it, bL, fM, dA, AS, eR, jx, eX, ew, aA, cz, v, j, bX, Vh, Zk, AO, pd, q, J, qH, eA, MR, mP, qK, aT, cy, qG, qv, Fx, ajh, Fg, ae, eu, qw, aii, aju, s, iU, OV, Y, Lf, ajx, r, lo, uU, en, bu, cl, pt, Mu, t, ho, pb, bN, ajq, ck, ajl, ik, BU, VJ, kP, nM, ajs, iM, gX, Da, dy, oX, fD, bt, am, ZI, aZ, aD, iW, VT, jh, acU, oZ, bS, oH, eq, hF, ad, dP, fU, av, hd, F, Cj, py, db, ajk, qh, oW, vc, vP, iL, Ng, d, je, ajr, BO, qt, Fm, ff, cr, eZ, dq, EH, xH, aF, EZ, Z, bz, dr, qa, adq, ek, bF, gk, bE, afd, ajw, MJ, rH, bC, lM, ajt, X, jr, BA); #c1(ATM) c2(XP_011541147) c3(serine-protein kinase ATM isoform X5) c4(339684) c5( ) c6( ) c7(bM, T, os, aC, fR, ea, if, e, al, hE, fd, ajp, bB, lR, ci, Lw, aY, aR, ip, x, bv, aji, aE, pX, ajv, M, aU, iv, K, hu, eL, bc, N, tw, zQ, iE, aW, WE, hl, adp, g, as, pl, eF, cm, bw, aj, qJ, y, eM, nB, iT, U, cj, k, m, pw, jM, iu, Ro, FW, dD, ajm, iB, aw, ht, ajg, n, cQ, aj, ct, qN, cd, br, ai, eW, b, ft, wG, i, gd, kH, h, eV, hn, dl, on, pl, Ck, OV, OS, RT, A, aq, kp, Hu, ajo, QK, OU, kT, W, ky, dB, hk, ajn, hL, MM, at, oh, WL, cB, B, it, bL, fM, dA, AS, eR, jx, eX, ew, aA, cz, v, j, bX, Vh, Zk, AO, pd, q, J, qH, eA, MR, mP, qK, aT, cy, qG, qv, Fx, ajh, Fg, ae, eu, qw, aii, aju, s, iU, oV, Y, Lf, ajx, r, lo, ul, en, bu, cl, pt, Mu, t, ho, pb, bN, ajq, ck, ajl, iK, BU, VJ, kP, nM, ajs, iM, gX, Da, dy, oX, fD, bt, am, ZI, aZ, aD, iW, VT, jh, acU, oZ, bS, OH, eq, hF, ad, dP, fU, av, hd, F, Cj, py, db, ajk, qh, oW, vc, vP, iL, Ng, d, je, ajr, BO, qt, Fm, ff, cr, eZ, dq, EH, xH, aF, EZ, Z, bz, dr, qa, adq, ek, bF, gk, bE, afd, ajw, MJ, rH, bC, lM, ajt, X, jr, BA); #c1(ATM) c2(XP_011541148) c3(serine-protein kinase ATM isoform X6) c4(339685, 339686) c5(339687, 339688, 339689, 339690, 339691, 339692, 339693, 339694) c6( ) c7(bM, T, os, aC, fR, ea, if, e, al, hE, fd, ajp, bB, lR, ci, Lw, aY, aR, ip, x, bv, aji, aE, pX, ajv, M, aU, iv, K, hu, eL, bc, N, tw, zQ, iE, aW, WE, hl, adp, g, as, pl, eF, cm, bw, aj, qJ, y, BM, nB, iT, U, cj, k, m, pw, jM, iu, Ro, FW, dD, ajm, iB, aw, ht, ajg, n, cd, aj, ct, qN, cd, br, ai, eW, b, ft, wG, i, gd, kH, h, eV, hn, dl, on, pl, Ck, OV, oS, RT, A, aq, kp, Hu, ajo, QK, OU, kT, W, ky, dB, hk, ajn, hL, MM, at, oh, WL, cB, B, it, bL, fM, dA, AS, eR, jx, eX, ew, aA, cz, v, j, bX, Vh, Zk, AO, pd, q, J, qH, eA, MR, mP, qK, aT, cy, qG, qv, Fx, ajh, Fg, ae, eu, qw, aii, aju, s, iU, oV, Y, Lf, ajx, r, lo, ul, en, bu, cl, pt, Mu, t, ho, pb, bN, ajq, ck, ajl, ik, BU, VJ, kP, nM, ajs, iM, gX, Da, dy, oX, fD, bt, am, ZI, aZ, aD, iW, VT, jh, acU, oZ, bS, OH, eq, hF, ad, dP, fU, av, hd, F, Cj, py, db, ajk, qh, oW, vc, vP, iL, Ng, d, je, ajr, BO, qt, Fm, ff, cr, eZ, dq, EH, xH, aF, EZ, Z, bz, dr, qa, adq, ek, bF, gk, bE, afd, ajw, MJ, rH, bC, lM, ajt, X, jr, BA); #c1(ATN1) c2(NP_001007027) c3(atrophin-1) c4(22405, 22406) c5(22407, 22408, 22409, 22410, 22411, 22412, 22413, 22414, 22415, 22416, 22417, 22418, 22419, 22420, 22421, 22422, 22423, 22424, 22425, 22426, 22427, 22428, 22429, 22430, 22431, 22432, 22433, 22434, 22435) c6( ) c7(oO, T, uk, if, fd, bB, ci, ajC, x, xy, aE, dV, K, ajK, ajA, dF, bc, eL, aW, Ls, eE, cm, bw, iP, ajH, ou, iT, Kv, vA, mx, U, cj, k, m, ajJ, hh, ik, ajy, yG, n, aj, Wb, ct, qN, eW, br, ajL, zw, ju, ajE, kH, h, eV, dL, yZ, on, HO, A, oo, Cu, lb, fe, aq, pm, RI, mo, ajz, ajB, op, NL, hL, pg, ajl, B, bL, Ob, fM, dA, jx, eX, rf, ajD, v, ajM, yU, q, J, er, eA, Kx, aT, rO, XU, ae, iU, oV, Y, r, en, hf, cl, ajG, ajF, t, f, ik, ck, cT, rR, iM, KB, lV, hc, AH, wR, mf, bt, ow, qb, aD, jN, jh, w, bS, dG, hF, Fc, iJ, ad, ds, ji, cb, fb, pq, je, lx, ff, eZ, Mz, dq, S, jg, iV, Z, iR, bz, dR, Ew, bE, Lj, uR, eB, Kj, rH, y); #c1(ATOH1) c2(NP_005163) c3(protein atonal homolog 1) c4(153536) c5(153537, 153538, 153539, 153540, 153541, 153542) c6( ) c7(jf, T, dD, s, bE, fm, ea, ev, X, aw, ajN, r, aA, bB, aF, eq, ci, g, iV, q, Yi); #c1(ATOH7) c2(NP_660161) c3(protein atonal homolog 7) c4(240461) c5(240462, 240463, 240464, 240465, 240466, 240467) c6( ) c7(HQ, cZ, T, yG, fd, ajQ, ad, ajO, ady, dV, aT, dd, ae, IR, dH, aW, Y, xs, ajP, cr, dw, cm, bw, bz, ac, U, cj, oQ, B); #c1(ATOX1) c2(NP_004036) c3(copper transport protein ATOX1) c4(143018) c5(143019, 143020, 143021, 143022, 143023, 143024, 143025, 143026, 143027, 143028, 143029, 143030, 143031, 143032) c6( ) c7(eY, T, aal, Js, ep, aS, m, Jo); #c1(ATP10A) c2(NP_077816) c3(probable phospholipid-transporting ATPase VA) c4(215393) c5(215394, 215395, 215396, 215397, 215398, 215399) c6( ) c7(bL, ac, dH, agl, fq, rc, bN); #c1(ATP10B) c2(NP_079429) c3(probable phospholipid-transporting ATPase VB) c4(218086) c5(218087, 218088, 218089, 218090, 218091, 218092) c6( ) c7(fq); #c1(ATP10D) c2(NP_065186) c3(probable phospholipid-transporting ATPase VD) c4(204487) c5(204488, 204489, 204490, 204491, 204492, 204493) c6( ) c7(ajR, aw, j); #c1(ATP10D) c2(XP_005248176) c3(probable phospholipid-transporting ATPase VD isoform X1) c4(268264) c5(268265, 268266, 268267, 268268, 268269, 268270) c6( ) c7(ajR, aw, j); #c1(ATP11A) c2(NP_056020) c3(probable phospholipid-transporting ATPase IH isoform a) c4(185179, 185180) c5(185181, 185182, 185183, 185184, 185185, 185186, 185187, 185188, 185189, 185190, 185191, 185192, 185193, 185194, 185195, 185196, 185197, 185198, 185199) c6( ) c7(eY, LB, ajS, ajT, aW, Y, mG, aT, kt, cd, bz, B, ajU); #c1(ATP11A) c2(NP_115565) c3(probable phospholipid-transporting ATPase IH isoform b) c4(223208) c5(223209, 223210, 223211, 223212, 223213, 223214) c6( ) c7(BY, LB, ajS, ajT, aW, Y, mG, aT, kt, cd, bz, B, ajU); #c1(ATP11A) c2(XP_005268356) c3(probable phospholipid-transporting ATPase IH isoform X4) c4( ) c5(281748, 281749) c6( ) c7(eY, LB, ajS, ajT, aW, Y, mG, aT, kt, cd, bz, B, ajU); #c1(ATP11A) c2(XP_011535781) c3(probable phospholipid-transporting ATPase IH isoform X3) c4( ) c5(332979) c6( ) c7(BY, LB, ajS, ajT, aW, Y, mG, aT, kt, cd, bz, B, ajU); #c1(ATP11A) c2(XP_011535783) c3 (probable phospholipid-transporting ATPase IH isoform X7) c4(332980, 332981) c5(332982, 332983, 332984, 332985, 332986, 332987, 332988, 332989, 332990, 332991, 332992) c6( ) c7(eY, LB, ajS, ajT, aW, Y, mG, aT, kt, cd, bz, B, ajU); #c1(ATP11AUN) c2(NP_997323) c3(putative protein ATP11AUN) c4(265404) c5(265405, 265406, 265407, 265408, 265409, 265410) c6( ) c7(bM); #c1(ATP11B) c2(NP_055431) c3(probable phospholipid-transporting ATPase IF) c4(181099) c5(181100, 181101, 181102, 181103, 181104, 181105) c6( ) c7(t, T, m); #c1(ATP11B) c2(XP_011510895) c3 (probable phospholipid-transporting ATPase IF isoform X2) c4(301363) c5(301364, 301365, 301366, 301367, 301368, 301369) c6( ) c7(t, T, m); #c1(ATP12A) c2(NP_001172014) c3(potassium-transporting ATPase alpha chain 2 isoform 1) c4(72236) c5(72237, 72238, 72239, 72240, 72241, 72242) c6( ) c7(T, akc, akd, aD, zs, ha, aA, aj, ci, ad, zw, yu, K, ajZ, ae, dl, ajV, VC, Nr, Mq, aiV, Y, az, kt, po, aF, aB, g, akb, ik, Nq, ajX, aka, mG, wR, ajY, VG, ajW, cE, B); #c1(ATP12A) c2(NP_001667) c3(potassium-transporting ATPase alpha chain 2 isoform 2) c4(119856) c5(119857, 119858, 119859, 119860, 119861, (19862) c6( ) c7(T, akc, akd, aD, zs, ha, aA, aj, ci, ad, zw, yu, K, ajZ, ae, dl, ajV, VC, Nr, Mq, aiV, Y, az, kt, po, aF, aB, g, akb, iK, Nq, ajX, aka, mG, wR, ajY, VG, ajW, cE, B); #c1(ATP13A3) c2(NP_078800) c3(probable cation-transporting ATPase 13A3) c4(215534, 215535) c5(215536, 215537, 215538, 215539, 215540, 215541, 215542, 215543, 215544, 215545, 215546, 215547, 215548, 215549, 215550, 215551) c6( ) c7(ci); #c1(ATP13A3) c2(XP_005269417) c3(probable cation-transporting ATPase 13A3 isoform X5) c4(282556) c5(282557, 282558, 282559, 282560, 282561, 282562, 282563) c6( ) c7(ci); #c1(ATP13A3) c2(XP_011511423) c3(probable cation-transporting ATPase 13A3 isoform X2) c4( ) c5(302066, 302067, 302068, 302069, 302070) c6( ) c7(ci); #c1(ATP13A3) c2(XP_011511424) c3(probable cation-transporting ATPase 13A3 isoform X3) c4(302071) c5( ) c6( ) c7(ci); #c1(ATP13A4) c2(NP_115655) c3(probable cation-transporting ATPase 13A4) c4(223456, 223457) c5(223458, 223459, 223460, 223461, 223462, 223463, 223464, 223465, 223466, 223467, 223468, 223469, 223470, 223471, 223472, 223473, 223474, 223475, 223476) c6( ) c7(akf, aC, ake, Kr); #c1(ATP13A4) c2(XP_011511534) c3(probable cation-transporting ATPase 13A4 isoform X2) c4(302249) c5( ) c6( ) c7(akf, aC, ake, Kr); #c1(ATP1A2) c2(NP_000693) c3(sodium/potassium-transporting ATPase subunit alpha-2 proprotein) c4(14565, 14566, 14567, 14568, 14569) c5(14570, 14571, 14572, 14573, 14574, 14575, 14576, 14577, 14578, 14579, 14580, 14581, 14582, 14583, 14584, 14585, 14586, 14587, 14588, 14589, 14590, 14591, 14592, 14593, 14594, 14595, 14596, 14597, 14598, 14599) c6( ) c7(akn, ha, cz, n, ur, uu, nA, akg, akk, ako, bK, akr, akl, XK, la, akh, vo, akf, akq, akp, aki, akm, akj, lw); #c1(ATP1A3) c2(NP_001243142) c3(sodium/potassium-transporting ATPase subunit alpha-3 isoform 2) c4(89988) c5(89989, 89990, 89991, 89992, 89993, 89994) c6( ) c7(xw, akv, akx, aki, Wk, akt, aU, bK, K, akh, aks, Wm, akw, aen, cd, aR, aku, dR); #c1(ATP1A3) c2(NP_001243143) c3(sodium/potassium-transporting ATPase subunit alpha-3 isoform 3) c4(89995, 89996, 89997, 89998) c5(89999, 90000, 90001, 90002, 90003, 90004, 90005, 90006, 90007, 90008, 90009, 90010, 90011, 90012, 90013, 90014, 90015, 90016, 90017, 90018, 90019, 90020, 90021, 90022, 90023) c6( ) c7(xw, akv, akx, aki, Wk, akt, aU, bK, K, akh, aks, Wm, akw, aen, cd, aR, aku, dR); #c1(ATP1A3) c2(NP_689509) c3(sodium/potassium-transporting ATPase subunit alpha-3 isoform 1) c4(242961, 242962) c5( ) c6( ) c7(xw, akv, akx, aki, Wk, akt, aU, bK, K, akh, aks, Wm, akw, aen, cd, aR, aku, dR); #c1(ATP1A3) c2(XP_011525293) c3(sodium/potassium-transporting ATPase subunit alpha-3 isoform X1) c4( ) c5(319759, 319760, 319761, 319762, 319763, 319764, 319765, 319766, 319767, 319768, 319769) c6( ) c7(xw, akv, akx, aki, Wk, akt, aU, bK, K, akh, aks, Wm, akw, aen, cd, aR, aku, dR); #c1(ATP1A4) c2(NP_001001734) c3(sodium/potassium-transporting ATPase subunit alpha-4 isoform 2) c4( ) c5(19007, 19008, 19009, 19010, 19011, 19012) c6( ) c7(aky, uT, MM, CR, cz, oJ, qa); #c1(ATP1A4) c2(NP_653300) c3(sodium/potassium-transporting ATPase subunit alpha-4 isoform 1) c4(239530, 239531) c5(239532, 239533, 239534, 239535, 239536, 239537) c6( ) c7(aky, uT, MM, CR, cz, oJ, qa); #c1(ATP1B1) c2(NP_001668) c3(sodium/potassium-transporting ATPase subunit beta-1) c4(119863, 119864, 119865) c5(119866, 119867, 119868, 119869, 119870, 119871, 119872, 119873, 119874, 119875, 119876, 119877, 119878, 119879, 119880, 119881, 119882, 119883, 119884, 119885, 119886, 119887) c6( ) c7(mG, bt, cz, EQ, lB, qa, bA, mu); #c1(ATP1B2) c2(NP_001669) c3(sodium/potassium-transporting ATPase subunit beta-2 isoform 1) c4(119888) c5(119889, 119890, 119891, 119892, 119893, 119894) c6( ) c7(T, aC, d, aW, qy, aT, at, dL, m, it, eA); #c1(ATPZA1) c2(NP_001273004) c3(sarcoplasmic/endoplasmic reticulum calcium ATPase 1 isoform c) c4( ) c5(104151, 104152, 104153, 104154, 104155) c6( ) c7(wY, yi, vX, lW, oj, akz, akB, akA, xk, K, lQ, fq, cd); #c1(ATPZA1) c2(NP_004311) c3(sarcoplasmic/ endoplasmic reticulum calcium ATPase 1 isoform a) c4(145074, 145075) c5(145076, 145077, 145078, 145079, 145080) c6( ) c7(wY, yi, vX, lW, oj, akz, akB, akA, xk, K, lL, fq, cd); #c1(ATPZA1) c2(NP_775293) c3(sarcoplasmic/endoplasmic reticulum calcium

ATPase 1 isoform b) c4(249879, 249880) c5(249881, 249882, 249883, 249884, 249885, 249886, 249887, 249888, 249889, 249890, 249891, 249892, 249893, 249894, 249895, 249896) c6( ) c7(wY, yi, vX, lW, oj, akz, akB, akA, xk, K, lQ, fq, cd); #c1(ATPZA2) c2(NP_001672) c3(sarcoplasmic/endoplasmic reticulum calcium ATPase 2 isoform a) c4(119902) c5(119903, 119904, 119905, 119906, (19907, 119908, 119909) c6( ) c7(bL, T, akD, cz, aj, akC, sl, agY, akG, cd, J, akE, cc, Bc, JD, vc, bK, K, lQ, bq, ff, wl, dN, en, mo, bQ, iK, akF, cl, cA, nF, ll, gZ, at, eo, bD, gT, cB, X, ao); #c1(ATP2A2) c2(NP_733765) c3(sarcoplasmic/endoplasmic reticulum calcium ATPase 2 isoform b) c4(247004, 247005, 247006) c5(247007, 247008, 247009, 247010, 247011, 247012, 247013, 247014, 247015, 247016, 247017, 247018, 247019, 247020, 247021, 247022, 247023, 247024, 247025) c6( ) c7(bL, T, akD, cz, aj, akC, sl, agY, akG, cd, J, akE, cc, Bc, JD, vc, bK, K, lQ, bq, ff, wl, dN, en, mo, bQ, ik, akF, cl, cA, nF, ll, gZ, at, eo, bD, gT, cB, X, ao); #c1(ATP2A3) c2(NP_777613) c3(sarcoplasmic/endoplasmic reticulum calcium ATPase 3 isoform e) c4(251326, 251327) c5(251328, 251329, 251330, 251331, 251332, 251333) c6( ) c7(bL, aA, cz, bB, aj, dG, ci, J, av, K, bq, s, ff, aF, en, g, bQ, ik, bF, ac, bE, akH, cE, X); #c1(ATPZA3) c2(NP_777615) c3(sarcoplasmic/endoplasmic reticulum calcium ATPase 3 isoform b) c4(251334) c5(251335, 251336, 251337, 251338, 251339, 251340) c6( ) c7(bL, aA, cz, bB, aj, dG, ci, J, av, K, bq, s, ff, aF, en, g, bQ, ik, bF, ac, bE, akH, cE, X); #c1(ATPZA3) c2(NP_777618) c3(sarcoplasmic/endoplasmic reticulum calcium ATPase 3 isoform c) c4(251341) c5(251342, 251343, 251344, 251345, 251346, 251347, 251348, 251349, 251350, 251351) c6( ) c7(bL, aA, cz, bB, aj, dG, ci, J, av, K, bq, s, ff, aF, en, g, bQ, ik, bF, ac, bE, akH, cE, X); #c1(ATP2A3) c2(XP_011522188) c3(sarcoplasmic/ endoplasmic reticulum calcium ATPase 3 isoform X9) c4(316792) c5(316793, 316794, 316795, 316796, 316797, 316798) c6( ) c7(bL, aA, cz, bB, aj, dG, ci, J, av, K, bq, s, ff, aF, en, g, bQ, ik, bF, ac, bE, akH, cE, X); #c1(ATP2A3) c2(XP_011522192) c3(sarcoplasmic/endoplasmic reticulum calcium ATPase 3 isoform X1) c4(316799) c5(316800, 316801, 316802, 316803, 316804, 316805) c6( ) c7(bL, aA, cz, bB, aj, dG, ci, J, av, K, bq, s, ff, aF, en, g, bQ, ik, bF, ac, bE, akH, cE, X); #c1(ATPZA3) c2(XP_011522193) c3(sarcoplasmic/endoplasmic reticulum calcium ATPase 3 isoform X2) c4(316806) c5(316807, 316808, 316809, 316810, 316811, 316812, 316813, 316814, 316815, 316816, 316817, 316818) c6( ) c7(bL, aA, cz, bB, aj, dG, ci, J, av, K, bq, s, ff, aF, en, g, bQ, ik, bF, ac, bE, akH, cE, X); #c1(ATP2B1) c2(XP_011536710) c3(plasma membrane calcium-transporting ATPase 1 isoform X1) c4(334271, 334272) c5(334273, 334274, 334275, 334276, 334277, 334278, 334279, 334280, 334281, 334282, 334283, 334284, 334285, 334286, 334287, 334288) c6( ) c7(bv, yi, aW, oe, cz, aT, j, K, ey, qa, dR); #c1(ATP2B1) c2(XP_011536712) c3(plasma membrane calcium-transporting ATPase 1 isoform X5) c4(334289, 334290) c5(334291, 334292, 334293, 334294, 334295, 334296) c6( ) c7(bv, yi, aW, oe, cz, aT, j, K, ey, qa, dR); #c1(ATP2B1) c2(XP_011536713) c3(plasma membrane calcium-transporting ATPase 1 isoform X2) c4(334297) c5(334298, 334299, 334300, 334301, 334302, 334303) c6( ) c7(bv, yi, aW, oe, cz, aT, j, K, ey, qa, dR); #c1(ATP2B1) c2(XP_011536714) c3(plasma membrane calcium-transporting ATPase 1 isoform X6) c4( ) c5(334304, 334305, 334306, 334307, 334308, 334309) c6( ) c7(bv, yi, aW, oe, cz, aT, j, K, ey, qa, dR); #c1(ATP2B2) c2(NP_001001331) c3(plasma membrane calcium-transporting ATPase 2 isoform 1) c4(18195, 18196) c5(18197, 18198, 18199, 18200, 18201, 18202, 18203, 18204, 18205, 18206, 18207, 18208, 18209, 18210, 18211, 18212) c6( ) c7(ac, KD, bE, aW, akJ, VB, cz, aT, bB, fe, akl, qa, bN); #c1(ATP2B2) c2(NP_001674) c3(plasma membrane calcium-transporting ATPase 2 isoform 2) c4(119910) c5(119911, 119912, 119913, 119914, 119915, 119916, 119917, 119918, 119919, 119920) c6( ) c7(ac, KD, bE, aW, akJ, VB, cz, aT, bB, fe, akl, qa, bN); #c1(ATP2B2) c2(XP_011532055) c3(plasma membrane calcium-transporting ATPase 2 isoform X3) c4(328111) c5( ) c6( ) c7(ac, KD, bE, aW, akJ, VB, cz, aT, bB, fe, akl, qa, bN); #c1(ATP2B2) c2(XP_011532056) c3(plasma membrane calcium-transporting ATPase 2 isoform X4) c4(328112) c5(328113, 328114, 328115, 328116, 328117, 328118) c6( ) c7(ac, KD, bE, aW, akJ, VB, cz, aT, bB, fe, akl, qa, bN); #c1(ATP2B2) c2(XP_011532057) c3 (plasma membrane calcium-transporting ATPase 2 isoform X5) c4(328119) c5( ) c6( ) c7(ac, KD, bE, aW, akJ, VB, cz, aT, bB, fe, akl, qa, bN); #c1(ATP2B2) c2(XP_011532058) c3(plasma membrane calcium-transporting ATPase 2 isoform X6) c4( ) c5(328120, 328121, 328122, 328123, 328124, 328125, 328126) c6( ) c7(ac, KD, bE, aW, akJ, VB, cz, aT, bB, fe, akl, qa, bN); #c1(ATP2B2) c2(XP_011532061) c3 (plasma membrane calcium-transporting ATPase 2 isoform X8) c4( ) c5(328127, 328128, 328129, 328130, 328131) c6( ) c7(ac, KD, bE, aW, akJ, VB, cz, aT, bB, fe, akl, qa, bN); #c1(ATP2B3) c2(XP_005274747) c3(plasma membrane calcium-transporting ATPase 3 isoform X2) c4(285363) c5(285364, 285365, 285366, 285367, 285368, 285369, 285370, 285371, 285372, 285373, 285374, 285375, 285376) c6( ) c7(qa, Fj, s, ky, bE, aW, xz, cz, aT, MM, bB, akL, akk); #c1(ATP2B3) c2(XP_005274749) c3(plasma membrane calcium-transporting ATPase 3 isoform X1) c4(285377, 285378, 285379) c5(285380, 285381, 285382, 285383, 285384, 285385, 285386, 285387, 285388, 285389, 285390, 285391, 285392, 285393, 285394, 285395, 285396, 285397, 285398, 285399, 285400, 285401) c6( ) c7(qa, Fj, s, ky, bE, aW, xz, cz, aT, MM, bB, akL, akK); #c1(ATP2B3) c2(XP_011529479) c3(plasma membrane calcium-transporting ATPase 3 isoform X5) c4( ) c5(324924, 324925) c6( ) c7(qa, Fj, s, ky, bE, aW, xz, cz, aT, MM, bB, akL, akK); #c1(ATP2B4) c2(NP_001001396) c3(plasma membrane calcium-transporting ATPase 4 isoform 4a) c4(18324, 18325, 18326, 18327, 18328) c5(18329, 18330, 18331, 18332, 18333, 18334, 18335, 18336, 18337, 18338, 18339, 18340, 18341, 18342, 18343, 18344, 18345, 18346, 18347, 18348, 18349, 18350, 18351, 18352, 18353, 18354, 18355, 18356) c6( ) c7(T, FG, aW, bE, dV, aT, aU, cz, bB, bQ, cd, bN); #c1(ATP2B4) c2(NP_001675) c3(plasma membrane calcium-transporting ATPase 4 isoform 4b) c4(119921) c5(119922, 119923, 119924, 119925, 119926, 119927, 119928, 119929, 119930, 119931, 119932, 119933) c6( ) c7(T, FG, aW, bE, dV, aT, aU, cz, bB, bQ, cd, bN); #c1(ATP2CI) c2(NP_001001486) c3(calcium-transporting ATPase type 2C member 1 isoform Id) c4(18509, 18510) c5(18511, 18512, 18513, 18514, 18515, 18516, 18517, 18518, 18519, 18520, 18521, 18522, 18523, 18524, 18525, 18526, 18527, 18528, 18529, 18530, 18531, 18532, 18533, 18534, 18535, 18536, 18537, 18538, 18539, 18540, 18541, 18542, 18543, 18544, 18545) c6( ) c7(T, A, dA, cA, bE, aW, ds, Y, aT, dN, h, gA, aj, K, lQ, pL, akG, bz, iu, J, akE); #c1(ATP2CI) c2(NP_001001487) c3(calcium-transporting ATPase type 2C member 1 isoform (b) c4(18546) c5(18547, 18548, 18549, 18550, 18551, 18552) c6( ) c7(T, A, dA, cA, bE, aW, ds, Y, aT, dN, h, gA, aj, K, lQ, pl, akG, bz, iu, J, akE); #c1(ATP2C1) c2(NP_001186109) c3(calcium-transporting ATPase type 2C member 1 isoform 2a) c4(77151) c5(77152, 77153, 77154, 77155, 77156, 77157, 77158, 77159) c6( ) c7(T, A, dA, cA, bE, aW, ds, Y, aT, dN, h, gA, aj, K, lQ, pl, akG, bz, iu, J, akE); #c1(ATP2C1) c2(NP_001186110) c3(calcium-transporting ATPase type 2C member 1 isoform 2b) c4( ) c5(77160) c6( ) c7(T, A, dA, cA, bE, aW, ds, Y, aT, dN, h, gA, aj, K, lQ, pl, akG, bz, iu, J, akE); #c1(ATP2CI) c2(NP_001186 μl) c3(calcium-transporting ATPase type 2C member 1 isoform 2c) c4(77161) c5(77162, 77163, 77164, 77165, 77166, 77167) c6( ) c7(T, A, dA, cA, bE, aW, ds, Y, aT, dN, h, gA, aj, K, lQ, pl, akG, bz, iu, J, akE); #c1(ATP2CI) c2(NP_001186112) c3(calcium-transporting ATPase type 2C member 1 isoform 1e) c4(77168) c5(77169, 77170, 77/71, 77172, 77173, 77174, 77175, 77176) c6( ) c7(T, A, dA, cA, bE, aW, ds, Y, aT, dN, h, gA, aj, K, lQ, pl, akG, bz, iu, J, akE); #c1(ATP2CI) c2(NP_001186114) c3(calcium-transporting ATPase type 2C member 1 isoform Ic) c4(77/77) c5(77178, 77179, 77180, 77181, 77182, 77183) c6( ) c7(T, A, dA, cA, bE, aW, ds, Y, aT, dN, h, gA, aj, K, lQ, pL, akG, bz, iu, J, akE); #c1(ATP2CI) c2(NP_055197) c3(calcium-transporting ATPase type 2C member 1 isoform 1a) c4(179718, 179719, 179720, 179721) c5( ) c6( ) c7(T, A, dA, cA, bE, aW, ds, Y, aT, dN, h, gA, aj, K, lQ, pl, akG, bz, iu, J, akE); #c1(ATP2CI) c2(XP_011510989) c3(calcium-transporting ATPase type 2C member 1 isoform X7) c4(301432) c5(301433, 301434, 301435, 301436, 301437, 301438, 301439, 301440, 301441, 301442, 301443, 301444, 301445, 301446, 301447, 301448, 301449, 301450, 301451) c6( ) c7(T, A, dA, cA, bE, aW, ds, Y, aT, dN, h, gA, aj, K, lQ, pl, akG, bz, iu, J, akE); #c1(ATP2C2) c2(NP_001273456) c3(calcium-transporting ATPase type 2C member 2 isoform 1) c4(104958) c5(104959, 104960, 104961, 104962, 104963, 104964) c6( ) c7(aW, VT, aT, yA, aha, vo); #c1(ATP2C2) c2(NP_001278383) c3(calcium-transporting ATPase type 2C member 2 isoform 3) c4(109604) c5(109605, 109606, 109607, 109608, 109609, 109610) c6( ) c7(aW, VT, aT, yA, aha, vo); #c1(ATP2C2) c2(NP_055676) c3(calcium-transporting ATPase type 2C member 2 isoform 2) c4(182679) c5(182680, 182681, 182682, 182683, 182684, 182685) c6( ) c7(aW, VT, aT, yA, aha, vo); #c1(ATP4A) c2(NP_000695) c3(potassium-transporting ATPase alpha chain 1) c4(14600) c5(14601, 14602, 14603, 14604, 14605, 14606) c6( ) c7(T, akc, akd, aD, zs, ha, aA, aj, zw, yu, K, ajZ, dl, ajV, VC, Nr, Mq, aiV, JI, Y, az, kt, po, aF, aB, akb, Nq, ajX, aka, mG, wR, ajY, VG, ajW, cE, B, Ex); #c1(ATP4B) c2(NP_000696) c3(potassium-transporting ATPase subunit beta) c4(14607) c5(14608, 14609, 14610, 14611, 14612, 14613) c6( ) c7(aF, e, j, bK, aA); #c1(ATP5A1) c2(NP_001001937) c3(ATP synthase subunit alpha, mitochondrial isoform a precursor) c4(19185, 19186) c5(19187, 19188, 19189, 19190, 19191, 19192, 19193, 19194, 19195, 19196, 19197, 19198, 19199) c6( ) c7(BN, Z, af, cA, r, bZ, bS, hy, ct, jo, K, pM, q, v, ne, ad, akM); #c1(ATP5A1) c2(NP_001244263) c3(ATP synthase subunit alpha, mitochondrial isoform b precursor) c4(92088, 92089) c5(92090, 92091, 92092, 92093, 92094, 92095, 92096, 92097, 92098, 92099, 92100) c6( ) c7(BN, Z, af, cA, r, bZ, bS, hy, ct, jo, K, pM, q, v, ne, ad, akM); #c1(ATP5A1) c2(XP_011524320) c3(ATP synthase subunit alpha, mitochondrial isoform X1) c4( ) c5(318809, 318810, 318811, 318812, 318813, 318814, 318815, 318816) c6( ) c7(BN, Z, af, cA, r, bZ, bS, hy, ct, jo, K, pM, q, v, ne, ad, akM); #c1(ATP5B) c2(NP_001677) c3(ATP synthase subunit beta, mitochondrial precursor) c4(119934) c5(119935, 119936, 119937, 119938, 119939, 119940) c6( ) c7(akN, cz); #c1(ATP5CI) c2(NP_001001973) c3(ATP synthase subunit gamma, mitochondrial isoform L (liver) precursor) c4(19230) c5(19231, 19232, 19233, 19234, 19235, 19236) c6( ) c7(q, ad, r); #c1(ATP5C1) c2(NP_005165) c3(ATP synthase subunit gamma, mitochondrial isoform H (heart) precursor) c4(153543, 153544) c5(153545, 153546, 153547, 153548, 153549, 153550, 153551) c6( ) c7(q, ad, r); #c1(ATP5CI) c2(XP_011517792) c3(ATP synthase subunit gamma, mitochondrial isoform X1) c4( ) c5(311670, 311671, 311672, 311673, 311674) c6( ) c7(q, ad, r); #c1(ATP5D) c2(NP_001001975) c3(ATP synthase subunit delta, mitochondrial precursor) c4(19253) c5(19254, 19255, 19256, 19257, 19258, 19259, 19260, 19261, 19262, 19263) c6( ) c7(g); #c1(ATP5E) c2(NP_008817) c3(ATP synthase subunit epsilon, mitochondrial) c4(168786, 168787) c5(168788, 168789, 168790, 168791, 168792, 168793, 168794, 168795, 168796, 168797, 168798, 168799, 168800, 168801, 168802, 168803, 168804, 168805, 168806, 168807) c6( ) c7(ajS, akQ, iU, hb, akO, oW, ahz, v, ci, cb, kC, X, ajU, akP); #c1(ATP5G1) c2(NP_005166) c3(ATP synthase F(0) complex subunit C1, mitochondrial precursor) c4(153552) c5(153553, 153554, 153555, 153556, 153557, 153558, 153559, 153560, 153561, 153562) c6( ) c7(rY); #c1(ATP5G2) c2(NP_001002031) c3(ATP synthase F(0) complex subunit C2, mitochondrial isoform a precursor) c4(19400) c5(19401, 19402, 19403, 19404, 19405, 19406, 19407, 19408, 19409, 19410, 19411) c6( ) c7(fr, ad); #c1(ATP5G2) c2(NP_005167) c3(ATP synthase F(0) complex subunit CZ, mitochondrial isoform b precursor) c4(153563) c5(153564, 153565, 153566, 153567, 153568, 153569, 153570) c6( ) c7(fr, ad); #c1(ATP5G2) c2(XP_011536788) c3(ATP synthase F(0) complex subunit CZ, mitochondrial isoform X1) c4(334466) c5( ) c6( ) c7(fr, ad); #c1(ATP5G3) c2(NP_001002258) c3(ATP synthase F(0) complex subunit C3, mitochondrial isoform A precursor) c4(19455) c5(19456, 19457, 19458, 19459, 19460, 19461, 19462, 19463, 19464, 19465, 19466, 19467, 19468, 19469) c6( ) c7(bv, iO, ahy, s, aW, dc, eH, aT, eF, B, bN); #c1(ATP5H) c2(NP_001003785) c3(ATP synthase subunit d, mitochondrial isoform b) c4(20086) c5(20087, 20088, 20089, 20090, 20091, 20092) c6( ) c7(iu, B); #c1(ATP5H) c2(NP_006347) c3(ATP synthase subunit d, mitochondrial isoform a) c4(164208, 164209) c5(164210, 164211, 164212, 164213, 164214, 164215, 164216, 164217, 164218, 164219, 164220, 164221) c6( ) c7(iu, B); #c1(ATP5J) c2(NP_001003696) c3(ATP synthase-coupling factor 6, mitochondrial isoform a precursor) c4(19964) c5(19965, 19966, 19967, 19968, 19969, 19970, 19971, 19972, 19973, 19974, 19975, 19976, 19977, 19978, 19979, 19980, 19981) c6( ) c7(eR, ajU, aw, cz, gZ, r, aj, j, q, J, ad); #c1(ATP5J) c2(NP_001003701) c3(ATP synthase-coupling factor 6, mitochondrial isoform b precursor) c4(19989) c5(19990, 19991, 19992, 19993, 19994, 19995, 19996, 19997, 19998, 19999, 20000) c6( ) c7(eR, aju, aw, cz, gZ, r, aj, j, q, J, ad); #c1(ATP5J2) c2(NP_001003713) c3(ATP synthase subunit f, mitochondrial isoform 2b) c4(20027, 20028) c5(20029, 20030, 20031, 20032, 20033, 20034, 20035, 20036, 20037, 20038, 20039, 20040) c6( ) c7(ad); #c1(ATP5J2) c2(NP_001003714) c3(ATP synthase subunit f, mitochondrial isoform 2c) c4(20041) c5(20042, 20043, 20044, 20045, 20046, 20047) c6( ) c7(ad); #c1(ATP5J2) c2(NP_001034267) c3(ATP synthase subunit f, mitochondrial isoform 2d) c4(31643) c5(31644, 31645, 31646, 31647, 31648, 31649) c6( ) c7(ad); #c1(ATP5J2) c2(NP_004880) c3(ATP synthase subunit f, mitochondrial isoform 2a) c4(150516) c5(150517, 150518, 150519, 150520, 150521, 150522) c6( ) c7(ad); #c1(ATP5L) c2(NP_006467) c3(ATP synthase subunit g, mitochondrial) c4(165417) c5(165418, 165419, 165420, 165421, 165422, 165423) c6( ) c7(hy, ne, BN); #c1(ATP50) c2(NP_001688) c3(ATP synthase subunit 0, mitochondrial precursor) c4(119994) c5(119995, 119996, 119997, 119998, 119999, 120000) c6( ) c7(bL, ad); #c1(ATP6API) c2(NP_001174) c3(V-type proton ATPase subunit SI precursor) c4(72423, 72424, 72425, 72426) c5(72427, 72428, 72429, 72430, 72431, 72432, 72433, 72434, 72435, 72436, 72437, 72438, 72439, 72440, 72441, 72442, 72443, 72444, 72445, 72446, 72447, 72448, 72449, 72450) c6( ) c7(T, Ke, lP, if, fd, ci, qN, LI, akW, eW, ad, hX, akR, acu, akU, ds, qp, eT, cU, SH, h, K, zy, ae, akS, akX, akT, akV, iU, ff, wc, Pg, lo, yq, xH, en, ck, kP, xD, Nk, i, bt); #c1(ATP6AP1L) c2(XP_011542025) c3(V-type proton ATPase subunit S1-like protein isoform X1) c4(340560) c5(340561, 340562, 340563, 340564, 340565, 340566, 340567, 340568, 340569, 340570) c6( ) c7(akY, c); #c1(ATP6AP2) c2(NP_005756) c3(renin receptor precursor) c4(159052, 159053, 159054, 159055, 159056, 159057, 159058, 159059) c5(159060, 159061, 159062, 159063, 159064, 159065, 159066, 159067, 159068, 159069, 159070, 159071, 159072, 159073, 159074, 159075, 159076, 159077, 159078, 159079, 159080, 159081, 159082, 159083, 159084, 159085, 159086, 159087, 159088, 159089, 159090, 159091, 159092, 159093, 159094, 159095, 159096, 159097, 159098, 159099, 159100, 159101, 159102, 159103, 159104, 159105, 159106, 159107) c6( ) c7(bL, T, eR, e, cz, aj, j, sl, aY, J, hX, fU, ws, sN, aT, ey, lQ, j, tw, ala, aW, Az, cr, eZ, fe, alb, si, eE, wU, W, Pa, vy, wR, akZ, dy, Hb, tr, aS, CZ, y, kS, vQ); #c1(ATP6VOA1) c2(NP_001123492) c3(V-type proton ATPase 116 kDa subunit a isoform 1 isoform a) c4(47189) c5(47190, 47191, 47192, 47193, 47194, 47195) c6( ) c7(bL, dD, aiV, ec, ea, dQ, hh, cz, aq, wR, j, eq, C, Z, B); #c1(ATP6VOA1) c2(NP_001123493) c3(V-type proton ATPase 116 kDa subunit a isoform 1 isoform b) c4(47196, 47197) c5(47198, 47199, 47200, 47201, 47202, 47203, 47204, 47205, 47206, 47207, 47208, 47209) c6( ) c7(bL, dD, aiV, ec, ea, dQ, hh, cz, aq, wR, j, eq, C, Z, B); #c1(ATP6VOA1) c2(NP_005168) c3(V-type proton ATPase 116 kDa subunit a isoform 1 isoform c) c4(153571, 153572) c5(153573, 153574, 153575, 153576, 153577, 153578, 153579, 153580, 153581, 153582, 153583, 153584) c6( ) c7(bL, dD, aiV, ec, ea, dQ, hh, cz, aq, wR, j, eq, C, Z, B); #c1(ATP6VOA1) c2(XP_005257516) c3(V-type proton ATPase 116 kDa subunit a isoform 1 isoform X1) c4(275192) c5(275193, 275194, 275195, 275196, 275197, 275198) c6( ) c7(bL, dD, aiV, ec, ea, dQ, hh, cz, aq, wR, j, eq, C, Z, B); #c1(ATP6VOA2) c2(NP_036595) c3(V-type proton ATPase 116 kDa subunit a isoform 2) c4(174685) c5(174686, 174687, 174688, 174689, 174690, 174691) c6( ) c7(gU, d, aiV, Uj, aW, ha, aT, alc, wR, ajW, C, ald, hh, Un, eA, dr); #c1(ATP6VOA2) c2(XP_005253620) c3(V-type proton ATPase 116 kDa subunit a isoform 2 isoform X1) c4(272599) c5(272600, 272601, 272602, 272603, 272604, 272605) c6( ) c7(gU, d, aiV, Uj, aW, ha, aT, alc, wR, ajW, C, ald, hh, Un, eA, dr); #c1(ATP6VOA4) c2(XP_005250451) c3(V-type proton ATPase 116 kDa subunit a isoform 4 isoform X1) c4(270308) c5(270309, 270310, 270311, 270312, 270313, 270314, 270315, 270316, 270317, 270318, 270319, 270320, 270321, 270322) c6( ) c7(py, W, aiV, qb, alf, mG, wR, alg, aj, C, hh, ale, Hp); #c1(ATP6VOC) c2(NP_001185498) c3(V-type proton ATPase 16 kDa proteolipid subunit) c4(75754) c5(75755, 75756, 75757, 75758, 75759, 75760) c6( ) c7(aW, T, B, aT); #c1(ATP6VOD1) c2(NP_004682) c3(V-type proton ATPase subunit d 1) c4(148806, 148807, 148808) c5(148809, 148810, 148811, 148812, 148813, 148814, 148815, 148816, 148817, 148818, 148819, 148820, 148821) c6( ) c7(hb, Pg); #c1(ATP6VOD1) c2(XP_011521749) c3(V-type proton ATPase subunit d 1 isoform X1) c4( ) c5(316456, 316457, 316458, 316459, 316460) c6( ) c7(hb, Pg); #c1(ATP6VOE1) c2(NP_003936) c3(V-type proton ATPase subunit e 1) c4(142321) c5(142322, 142323, 142324, 142325, 142326, 142327, 142328, 142329, 142330, 142331) c6( ) c7(aiW, akd, oj); #c1(ATP6VOE2) c2(NP_001094062) c3(V-type proton ATPase subunit e 2 isoform 2) c4(40571, 40572) c5(40573, 40574, 40575, 40576, 40577, 40578, 40579, 40580, 40581, 40582, 40583, 40584, 40585, 40586, 40587) c6( ) c7(de); #c1(ATP6VOE2) c2(NP_001276919) c3(V-type proton ATPase subunit e 2 isoform 3) c4(108625) c5(108626, 108627, 108628, 108629, 108630, 108631, 108632, 108633, 108634, 108635) c6( ) c7(De); #c1(ATP6VOE2) c2(NP_660265) c3(V-type proton ATPase subunit e 2 isoform 1) c4(240573, 240574) c5(240575, 240576, 240577, 240578, 240579, 240580, 240581, 240582, 240583, 240584) c6( ) c7(ce); #c1(ATP6V1B1) c2(NP_001683) c3(V-type proton ATPase subunit B, kidney isoform) c4(119941, 119942, 119943) c5(119944, 119945, 119946, 119947, 119948, 119949, 119950, 119951, 119952, 119953, 119954, 119955, 119956, 119957, 119958, 119959, 119960, 119961) c6( ) c7(ajT, alf, zs, mG, ali, cz, alh, ale); #c1(ATP6VIB2) c2(NP_001684) c3(V-type proton ATPase subunit B, brain isoform) c4(19962, 119963) c5(119964, 119965, 119966, 119967, 119968, 119969, 119970, 119971, 119972, 119973, 119974, 119975) c6( ) c7(kf, he, ey); #c1(ATP6VICI) c2(NP_001686) c3(V-type proton ATPase subunit C 1) c4(119976) c5(119977, 119978, 119979, 119980, 119981, 119982, 119983, 119984, 119985, 119986) c6( ) c7(T, ac, BN, aW, ff, aT, hy, en, ne); #c1(ATP6VID) c2(NP_057078) c3(V-type proton ATPase subunit D) c4(188829, 188830) c5(188831, 188832, 188833, 188834, 188835, 188836, 188837, 188838, 188839, 188840, 188841, 188842, 188843, 188844, 188845, 188846) c6( ) c7(at); #c1(ATP6V1E1) c2(NP_001034455) c3(V-type proton ATPase subunit E 1 isoform b) c4(31732) c5(31733, 31734, 31735, 31736, 31737, 31738) c6( ) c7(ib, bk, hX); #c1(ATP6V1E1) c2(NP_001034456) c3(V-type proton ATPase subunit E 1 isoform c) c4(31739) c5(31740, 31741, 31742, 31743, 31744, 31745) c6( ) c7(ib, bk, hX); #c1(ATP6V1E1) c2(NP_001687) c3(V-type proton ATPase subunit E 1 isoform a) c4(119987) c5(119988, 119989, 119990, 119991, 119992, 119993) c6( ) c7(ib, bK, hX); #c1(ATP6V1F) c2(NP_001185838) c3(V-type proton ATPase subunit F isoform 2) c4(76563) c5(76564, 76565, 76566, 76567, 76568, 76569) c6( ) c7(ae); #c1(ATP6V1F) c2(NP_004222) c3(V-type proton ATPase subunit F isoform 1) c4(144417) c5(144418, 144419, 144420, 144421, 144422, 144423) c6( ) c7(ae); #c1(ATP6V1G1) c2(NP_004879) c3(V-type proton ATPase subunit G 1) c4(150509) c5(150510, 150511, 150512, 150513, 150514, 150515) c6( ) c7(y, ac); #c1(ATP6VIG2) c2(NP_001191007) c3(V-type proton ATPase subunit G 2 isoform c) c4(80518) c5(80519, 80520, 80521, 80522, 80523, 80524, 80525, 80526, 80527, 80528, 80529, 80530, 80531, 80532, 80533, 80534, 80535, 80536, 80537, 80538, 80539, 80540, 80541, 80542, 80543) c6( ) c7(dV, eZ, aiJ, Kh, hH, y); #c1(ATP6VIG2) c2(NP_569730) c3(V-type proton ATPase subunit G 2 isoform a) c4(233840, 233841, 233842, 233843) c5(233844, 233845, 233846, 233847, 233848, 233849, 233850, 233851, 233852, 233853, 233854, 233855, 233856, 233857, 233858, 233859, 233860, 233861, 233862, 233863, 233864, 233865, 233866, 233867, 233868, 233869, 233870, 233871, 233872, 233873, 233874, 233875, 233876, 233877, 233878, 233879, 233880, 233881, 233882) c6( ) c7(dV, eZ, aiJ, Kh, hH, y); #c1(ATP6VIG2) c2(NP_612139) c3(V-type proton ATPase subunit G 2 isoform b) c4( ) c5(236008, 236009, 236010, 236011, 236012) c6( ) c7(dV, eZ, aiJ, Kh, hH, y); #c1(ATP6VIG3) c2(XP_006725021) c3(V-type proton ATPase subunit G 3 isoform X2) c4(296515) c5(296516, 296517, 296518, 296519, 296520, 296521, 296522, 296523, 296524, 296525) c6( ) c7(py); #c1(ATP6VIG3) c2(XP_011545238) c3(V-type proton ATPase subunit G 3 isoform X1) c4(344034) c5(344035, 344036, 344037, 344038, 344039, 344040, 344041, 344042, 344043, 344044, 344045) c6( ) c7(py); #c1(ATP6VIG3) c2(XP_011545239) c3(V-type proton ATPase subunit G 3 isoform X3) c4(344046) c5(344047, 344048, 344049, 344050, 344051) c6( ) c7(py); #c1(ATP7A) c2(NP_000043) c3(copper-transporting ATPase 1 isoform 1) c4(734, 735) c5(736, 737, 738, 739, 740, 741, 742) c6( ) c7(T, dD, Ol, ea, yk, ha, fd, bB, Mi, eq, Eh, q, alp, ep, Ak, aln, bV, alk, r, sm, aF, t, alj, cm, eY, alm, eM, aU, bE, Xp, U, wR, m, alo, Mj, X); #c1(ATP7A) c2(NP_001269153) c3(copper-transporting ATPase 1 isoform 2) c4(100713, 100714, 100715, 100716) c5(100717, 100718, 100719, 100720, 100721, 100722, 100723, 100724, 100725, 100726, 100727, 100728, 100729, 100730, 100731, 100732, 100733, 100734, 100735, 100736, 100737, 100738, 100739, 100740, 100741, 100742, 100743, 100744, 100745) c6( ) c7(T, dD, Ol, ea, yk, ha, fd, bB, Mi, eq, Eh, q, alp, ep, Ak, aln, bV, alk, r, sm, aF, t, alj, cm, eY, alm, eM, aU, bE, Xp, U, wR, m, alo, Mj, X); #c1(ATP7B) c2(NP_001005918) c3(copper-transporting ATPase 2 isoform b) c4(22167) c5(22168, 22169, 22170, 22171, 22172, 22173, 22174, 22175) c6(22176, 22177) c7(T, am, al, bO, ci, bX, q, aR, alr, av, ep, aT, aU, K, aln, als, ae, N, na, d, ff, fc, hl, r, alt, aq, en, iR, t, xw, bz, iK, dr, eY, es, iO, aaQ, Xp, m, alq, B, jr, ek); #c1(ATP7B) c2(NP_001230111) c3(copper-transporting ATPase 2 isoform c) c4(85939) c5(85940, 85941, 85942, 85943, 85944, 85945, 85946, 85947) c6(85948, 85949) c7(T, am, al, bO, ci, bX, q, aR, alr, av, ep, aT, aU, K, aln, als, ae, N, na, d, ff, fc, hl, r, alt, aq, en, iR, t, xw, bz, iK, dr, eY, es, iO, aaQ, Xp, m, alq, B, jr, ek); #c1(ATP7B) c2(XP_005266484) c3(copper-transporting ATPase 2 isoform X6) c4(280070) c5(280071, 280072, 280073, 280074, 280075, 280076, 280077, 280078) c6(280079, 280080) c7(T, am, al, bO, ci, bX, q, aR, alr, av, ep, aT, aU, K, aln, als, ae, N, na, d, ff, fc, hl, r, alt, aq, en, iR, t, xw, bz, iK, dr, eY, es, iO, aaQ, Xp, m, alq, B, jr, ek); #c1(ATP7B) c2(XP_005266485) c3(copper-transporting ATPase 2 isoform X7) c4(280081) c5( ) c6( ) c7(T, am, al, bO, ci, bX, q, aR, alr, av, ep, aT, aU, K, aln, als, ae, N, na, d, ff, fc, hl, r, alt, aq, en, iR, t, xw, bz, iK, dr, eY, es, iU, aaQ, Xp, m, alq, B, jr, ek); #c1(ATP7B) c2(XP_005266487) c3(copper-transporting ATPase 2 isoform X1) c4(280082) c5(280083, 280084, 280085, 280086, 280087, 280088, 280089, 280090, 280091, 280092, 280093, 280094, 280095, 280096, 280097, 280098, 280099, 280100, 280101, 280102, 280103, 280104, 280105, 280106, 280107, 280108, 280109, 280110, 280111, 280112, 280113, 280114, 280115, 280116, 280117, 280118, 280119, 280120, 280121, 280122, 280123, 280124, 280125, 280126, 280127, 280128, 280129, 280130, 280131, 280132, 280133, 280134, 280135, 280136, 280137, 280138, 280139, 280140, 280141, 280142, 280143, 280144 280145, 280146, 280147, 280148, 280149, 280150, 280151, 280152, 280153, 280154, 280155, 280156, 280157, 280158, 280159, 280160, 280161, 280162, 280163, 280164, 280165, 280166, 280167, 280168, 280169, 280170, 280171, 280172, 280173, 280174, 280175, 280176, 280177) c6(280178, 280179, 280180) c7(T, am, al, bO, ci, bX, q, aR, alr, av, ep, aT, aU, K, aln, als, ae, N, na, d, ff, fc, hl, r, alt, aq, en, iR, t, xw, bz, iK, dr, eY, es, iU, aaQ, Xp, m, alq, B, jr, ek); #c1(ATP7B) c2(XP_005266489) c3(copper-transporting ATPase 2 isoform X9) c4(280181) c5(280182, 280183, 280184, 280185, 280186, 280187, 280188, 280189) c6(280190, 280191) c7(T, am, al, bO, ci, bX, q, aR, alr, av, ep, aT, aU, K, aln, als, ae, N, na, d, ff, fc, hl, r, alt, aq, en, iR, t, xw, bz, iK, dr, eY, es, iO, aaQ, Xp, m, alq, B, jr, ek); #c1(ATP7B) c2(XP_006719902) c3(copper-transporting ATPase 2 isoform X13) c4( ) c5(293059, 293060, 293061, 293062, 293063, 293064, 293065) c6(293066, 293067) c7(T, am, al, bO, ci, bX, q, aR, alr, av, ep, aT, aU, K, aln, als, ae, N, na, d, ff, fc, hl, r, alt, aq, en, iR, t, xw, bz, ik, dr, eY, es, iO, aaQ, Xp, m, alq, B, jr, ek); #c1(ATP7B) c2(XP_011533420) c3(copper-transporting ATPase 2 isoform X4) c4(329515) c5(329516, 329517, 329518, 329519, 329520, 329521, 329522, 329523) c6(329524, 329525) c7(T, am, al, bO, ci, bX, q, aR, alr, av, ep, aT, aU, K, aln, als, ae, N, na, d, ff, fc, hl, r, alt, aq, en, iR, t, xw, bz, iK, dr, eY, es, iO, aaQ, Xp, m, alq, B, jr, ek); #c1(ATP7B) c2(XP_011533421) c3(copper-transporting ATPase 2 isoform X5) c4(329526) c5(329527) c6( ) c7(T, am, al, bO, ci, bX, q, aR, alr, av, ep, aT, aU, K, aln, als, ae, N, na, d, ff, fc, hl, r, alt, aq, en, iR, t, xw, bz, iK, dr, eY, es, iO, aaU, Xp, m, alq, B, jr, ek); #c1(ATP7B) c2(XP_011533423) c3(copper-transporting ATPase 2 isoform X10) c4( ) c5(329528, 329529, 329530, 329531, 329532, 329533, 329534, 329535) c6(329536, 329537) c7(T, am, al, bO, ci, bX, q, aR, alr, av, ep, aT, aU, K, aln, als, ae, N, na, d, ff, fc, hl, r, alt, aq, en, iR, t, xw, bz, iK, dr, eY, es, iO, aaQ, Xp, m, alq, B, jr, ek); #c1(ATP8A1) c2(NP_001098999) c3(phospholipid-transporting ATPase IA isoform b) c4(41588) c5(41589, 41590, 41591, 41592, 41593, 41594, 41595) c6( ) c7(bL, aj, hJ, agl, aw); #c1(ATP8A1) c2(NP_006086) c3(phospholipid-transporting ATPase IA isoform a) c4(161802) c5(161803, 161804, 161805, 161806, 161807, 161808) c6( ) c7(bL, aj, hJ, agl, aw); #c1(ATP8A2) c2(NP_057613) c3(phospholipid-transporting ATPase IB) c4(191754, 191755) c5(191756, 191757, 191758, 191759, 191760, 191761, 191762, 191763, 191764, 191765, 19/766, 191767) c6( ) c7(alu, nA, iX); #c1(ATP8A2) c2(XP_011533405) c3(phospholipid-transporting ATPase IB isoform X1) c4(329505) c5(329506) c6( ) c7(alu, nA, iX); #c1(ATP8A2) c2(XP_011533415) c3(phospholipid-transporting ATPase IB isoform X9) c4(329507) c5( ) c6( ) c7(alu, nA, iX); #c1(ATP8A2) c2(XP_011533416) c3(phospholipid-transporting ATPase IB isoform X10) c4( ) c5(329508, 329509, 329510, 329511, 329512, 329513) c6( ) c7(alu, nA, iX); #c1(ATP8B1) c2(XP_011524324) c3(phospholipid-transporting ATPase IC isoform X1) c4(318817) c5(318818, 318819, 318820, 318821, 318822, 318823, 318824, 318825, 318826, 318827, 318828, 318829, 318830, 318831, 318832, 318833, 318834, 318835, 318836, 318837, 318838, 318839, 318840, 318841, 318842, 318843, 318844, 318845, 318846, 318847, 318848, 318849, 318850, 318851, 318852, 318853, 318854, 318855, 318856, 318857, 318858, 318859, 318860, 318861, 318862, 318863, 318864, 318865, 318866, 318867, 318868, 318869, 318870, 318871, 318872, 318873, 318874, 318875, 318876, 318877, 318878, 318879, 318880, 318881, 318882, 318883, 318884, 318885, 318886, 318887, 318888, 318889, 318890, 318891, 318892, 318893, 318894, 318895, 318896, 318897, 318898, 318899, 318900, 318901, 318902, 318903, 318904, 318905, 318906, 318907, 318908, 318909, 318910, 318911, 318912, 318913, 318914, 318915, 318916, 318917, 318918, 318919, 318920, 318921, 318922, 318923, 318924, 318925, 318926, 318927, 318928, 318929, 318930, 318931, 318932, 318933, 318934, 318935, 318936, 318937, 318938, 318939, 318940, 318941, 318942, 318943, 318944, 318945, 318946, 318947, 318948, 318949, 318950, 318951, 318952, 318953, 318954, 318955, 318956, 318957, 318958, 318959, 318960, 318961, 318962, 318963, 318964, 318965, 318966, 318967, 318968, 318969, 318970, 318971, 318972, 318973, 318974, 318975, 318976, 318977, 318978, 318979, 318980, 318981, 318982, 318983, 318984, 318985, 318986, 318987, 318988, 318989, 318990, 318991, 318992, 318993, 318994, 318995, 318996, 318997, 318998, 318999, 319000) c6( ) c7(jq, ar, aC, iO, acw, hl, jN, jH, alv, j, alw, qj, hJ, jw, alx, ao, jK); #c1(ATP8B3) c2(NP_001171473) c3(phospholipid-transporting ATPase IK isoform 2) c4(70233) c5(70234, 70235, 70236, 70237, 70238) c6( ) c7(bK); #c1(ATP8B3) c2(NP_620168) c3(phospholipid-transporting ATPase IK isoform 1) c4(237444, 237445) c5(237446, 237447, 237448, 237449, 237450, 237451) c6( ) c7(bK); #c1(ATP8B3) c2(XP_006722718) c3(phospholipid-transporting ATPase IK isoform X1) c4(294854) c5(294855, 294856, 294857, 294858, 294859, 294860, 294861) c6( ) c7(bK); #c1(ATP8B3) c2(XP_006722720) c3(phospholipid-transporting ATPase IK isoform X8) c4( ) c5(294862, 294863, 294864, 294865, 294866, 294867) c6( ) c7(bK); #c1(ATP8B4) c2(NP_079113) c3 (probable phospholipid-transporting ATPase IM) c4( ) c5(217084, 217085, 217086, 217087, 217088, 217089, 217090, 217091) c6( ) c7(af, ac, j); #c1(ATP8B4) c2(XP_006720756) c3(probable phospholipid-transporting ATPase IM isoform X2) c4(293676) c5(293677, 293678) c6( ) c7(af, ac, j); #c1(ATP884) c2(XP_011520348) c3(probable phospholipid-transporting ATPase IM isoform X1) c4( ) c5(315193, 315194, 315195, 315196, 315197) c6( ) c7(af, ac, j); #c1(ATP8B4) c2(XP_011520356) c3(probable phospholipid-transporting ATPase IM isoform X4) c4(315198) c5( ) c6( ) c7(af, ac, j); #c1(ATP9B) c2(NP_940933) c3(probable phospholipid-transporting ATPase IIB) c4(260130) c5(260131, 260132, 260133, 260134, 260135, 260136) c6( ) c7(cu); #c1(ATP9B) c2(XP_011524263) c3(probable phospholipid-transporting ATPase IIB isoform X6) c4(318755) c5( ) c6( ) c7(cu); #c1(ATP9B) c2(XP_011524266) c3(probable phospholipid-transporting ATPase IIB isoform X9) c4(318756) c5( ) c6( ) c7(cu); #c1(ATP9B) c2(XP_011524270) c3(probable phospholipid-transporting ATPase IB isoform X14) c4(318757) c5( ) c6( ) c7(cu); #c1(ATP9B) c2(XP_011524278) c3(probable phospholipid-transporting ATPase IIB isoform X22) c4( ) c5(318758, 318759, 318760, 318761, 318762, 318763, 318764, 318765, 318766, 318767) c6( ) c7(cu); #c1(ATP9B) c2(XP_011524279) c3 (probable phospholipid-transporting ATPase IIB isoform X23) c4( ) c5(318768, 318769, 318770, 318771, 318772) c6( ) c7(cu); #c1(ATPAF2) c2(NP_663729) c3(ATP synthase mitochondrial Fl complex assembly factor 2) c4(241456) c5(241457, 241458, 241459, 241460, 241461, 241462) c6( ) c7(aly, alz, akP); #c1(ATPAF2) c2(XP_005256905) c3(ATP synthase mitochondrial Fl complex assembly factor 2 isoform X5) c4( ) c5(274565, 274566, 274567, 274568, 274569, 274570, 274571, 274572, 274573, 274574, 274575, 274576) c6( ) c7(aly, alz, akP); #c1(ATPAF2) c2(XP_011522365) c3(ATP synthase mitochondrial Fl complex assembly factor 2 isoform X2) c4(317046, 317047) c5( ) c6( ) c7(aly, alz, akP); #c1(ATR) c2(NP_001175) c3(serine/threonine-protein kinase ATR) c4(72451) c5(72452, 72453, 72454, 72455, 72456, 72457) c6( ) c7(T, os, dD, aC, id, eR, ea, WL, aw, ew, aA, bB, jh, oZ, j, eq, nA, WC, q, eA, eW, hb, KN, kH, aT, h, ey, K, WF, aij, alA, sb, d, kj, wr, aW, WE, Y, ff, alB, r, nf, kp, aF, en, cl, gW, t, QK, g, dr, op, ck, alC, WH, qe, eM, bE, vA, hL, cj, Km, m, ER, bC, WI, bt, X, BA); #c1(ATR) c2(XP_011511227) c3(serine/threonine-protein kinase ATR isoform X2) c4(30/706) c5(301707, 30/708, 301709, 301710, 301711, 301712) c6( ) c7(T, os, dD, aC, id, eR, ea, WL, aw, ew, aA, bB, jh, oZ, j, eq, nA, WC, q, eA, eW, hb, KN, KH, aT, h, ey, K, WF, aij, alA, sb, d, kj, wr, aW, WE, Y, ff, alB, r, nf, kp, aF, en, cl, gW, t, QK, g, dr, op, ck, alC, WH, qe, eM, bE, vA, hL, cj, Km, m, ER, bC, WI, bt, X, BA); #c1(ATRA1D) c2(NP_00164266) c3(all-trans retinoic acid-induced differentiation factor isoform c precursor) c4( ) c5(66893) c6( ) c7(ck, eM, Y, cn, iS, fd, bw, v, ci, K, hF, vt, B, ip); #c1(ATRA1D) c2(NP_057169) c3(all-trans retinoic acid-induced differentiation factor isoform a) c4( ) c5(189282, 189283, 189284, 189285, 189286, 189287, 189288, 189289, 189290, 189291) c6( ) c7(ck, eM, Y, cn, iS, fd, bw, v, ci, K, hF, vt, B, ip); #c1(ATRA1D) c2(NP_542159) c3(all-trans retinoic acid-induced differentiation factor isoform b) c4(231459, 231460, 231461) c5(231462, 231463, 231464, 231465, 231466, 231467, 231468, 231469) c6( ) c7(ck, eM, Y, cn, iS, fd, bw, v, ci, K, hF, vt, B, ip); #c1(ATR1P) c2(NP_001257952) c3(ATR-interacting protein isoform 4) c4(95857, 95858, 95859) c5(95860, 95861, 95862, 95863, 95864) c6( ) c7(T, Ez, dt, WE, aT, wR, K, m, t, Ex); #c1(ATR1P) c2(NP_115542) c3(ATR-interacting protein isoform 2) c4(223129) c5(223130, 223131, 223132, 223133, 223134, 223135, 223136) c6( ) c7(T, Ez, dt, WE, aT, wR, K, m, t, Ex); #c1(ATR1P) c2(NP_569055) c3(ATR-interacting protein isoform 1) c4(233578, 233579) c5(233580, 233581, 233582, 233583, 233584, 233585, 233586, 233587, 233588, 233589, 233590, 233591, 233592, 233593, 233594, 233595, 233596, 233597, 233598, 233599, 233600, 233601, 233602) c6( ) c7(T, Ez, dt, WE, aT, wR, K, m, t, Ex); #c1(ATRN) c2(NP_647537) c3(attractin isoform 1 preproprotein) c4(238693, 238694) c5(238695, 238696, 238697, 238698, 238699, 238700, 238701) c6( ) c7(alD, sS, cc, hk, Y, yk, afQ, ff, qy, v, en, as, eA); #c1(ATRN) c2(NP_647538) c3(attractin isoform 2 preproprotein) c4(238702) c5(238703, 238704, 238705, 238706, 238707, 238708) c6( ) c7(alD, sS, cc, hk, Y, yk, afQ, ff, qy, v, en, as, eA); #c1(ATRN) c2(XP_005260917) c3(attractin isoform X1) c4( ) c5(276967, 276968, 276969, 276970, 276971) c6( ) c7(alD, sS, cc, hk, Y, yk, afQ, ff, qy, v, en, as, eA); #c1(ATRNL1) c2(NP_997186) c3(attractin-like protein 1 isoform 1 precursor) c4(265133, 265134) c5(265135, 265136, 265137, 265138, 265139, 265140) c6( ) c7(TI, vT, HY, TH, TG, TJ, bE, eH, au, fd, bB, aq, rx, aaw, h, eF, y, nJ, B, bc); #c1(ATRNL1) c2(XP_011537889) c3(attractin-like protein 1 isoform X2) c4(335411) c5(335412, 335413, 335414, 335415, 335416, 335417, 335418) c6( ) c7(TI, vT, HY, TH, TG, TJ, bE, eH, au, fd, bB, aq, rx, aaw, h, eF, y, nJ, B, bc); #c1(ATRNL1) c2(XP_011537893) c3(attractin-like protein 1 isoform X5) c4( ) c5(335419, 335420, 335421, 335422, 335423) c6( ) c7(TI, vT, HY, TH, TG, TJ, bE, eH, au, fd, bB, aq, rx, aaw, h, eF, y, nJ, B, bc); #c1(ATRX) c2(NP_000480) c3(transcriptional regulator ATRX isoform 1) c4(9645) c5(9646, 9647, 9648, 9649, 9650, 9651, 9652) c6( ) c7(T, aC, pY, ha, bs, alH, ci, nA, q, br, eA, ip, eW, aE, pX, qK, aT, K, jj, alF, alG, WF, alk, yi, d, alJ, kS, aW, eH, Az, fc, r, nf, as, eF, t, cm, bw, aU, bN, ck, alE, Qi, BU, YE, WH, qe, dB, U, cj, iS, m, ER, cP, xb, st); #c1(ATRX) c2(NP_612114) c3 (transcriptional regulator ATRX isoform 2) c4(235871) c5(235872, 235873, 235874, 235875, 235876, 235877) c6( ) c7(T, aC, pY, ha, bs, alH, ci, nA, q, br, eA, ip, eW, aE, pX, qK, aT, K, jj, alF, alG, WF, alk, yi, d, alJ, kS, aW, eH, Az, fc, r, nf, as, eF, t, cm, bw, aU, bN, ck, alE, Qi, BU, YE, WH, qe, dB, U, cj, iS, m, ER, cP, xb, st); #c1(ATRX) c2(XP_005262211) c3(transcriptional regulator ATRX isoform X2) c4( ) c5(277785) c6( ) c7(T, aC, pY, ha, bs, alH, ci, nA, q, br, eA, ip, eW, aE, pX, qK, aT, K, jj, alF, alG, WF, alk, yi, d, alJ, kS, aW, eH, Az, fc, r, nf, as, eF, t, cm, bw, aU, bN, ck, alE, Qi, BU, YE, WH, qe, dB, U, cj, iS, m, ER, cP, xb, st); #c1(ATRX) c2(XP_005262214) c3(transcriptional regulator ATRX isoform X6) c4(277786) c5(277787, 277788, 277789, 277790, 277791, 277792) c6( ) c7(T, aC, pY, ha, bs, alH, ci, nA, q, br, eA, ip, eW, aE, pX, qK, aT, K, jj, alF, alG, WF, alk, yi, d, alJ, kS, aW, eH, Az, fc, r, nf, as, eF, t, cm, bw, aU, bN, ck, alE, Qi, BU, YE, WH, qe, dB, U, cj, iS, m, ER, cP, xb, st); #c1(ATRX) c2(XP_006724731) c3 (transcriptional regulator ATRX isoform X8) c4(296278, 296279, 296280) c5(296281, 296282, 296283, 296284, 296285, 296286, 296287, 296288, 296289, 296290, 296291, 296292, 296293, 296294, 296295, 296296, 296297) c6( ) c7(T, aC, pY, ha, bs, aU, ci, nA, q, br, eA, ip, eW, aE, pX, qK, aT, K, jj, alF, alG, WF, alk, yi, d, alJ, kS, aW, eH, Az, fc, r, nf, as, eF, t, cm, bw, aU, bN, ck, alE, Qi, BU, YE, WH, qe, dB, U, cj, iS, m, ER, cP, xb, st); #c1(ATXN1) c2(NP_001121636) c3(ataxin-1) c4(45939) c5(45940, 45941, 45942, 45943, 45944, 45945, 45946, 45947, 45948, 45949, 45950, 45951, 45952, 45953) c6( ) c7(T, os, iH, Js, aw, yY, rf, fd, lb, GC, oZ, v, j, Kh, akg, nA, Vd, fu, pd, cd, aR, oT, Yc, fm, aT, Ak, aU, cy, yZ, bK, K, jj, an, he, on, aU, af, aU, alM, iU, akL, dg, EH, RI, fq, dr, dR, ck, alN, KE, rR, W, ky, kB, c, dB, ac, Kv, U, nw, aS, Kj, bt, Jo); #c1(ATXN10) c2(NP_001161093) c3(ataxin-10 isoform 2) c4(65314) c5(65315, 65316, 65317, 65318, 65319, 65320) c6( ) c7(alP, ky, kB, ha, U, rf, vm, aan, aU, dr, wT, dR); #c1(ATXN10) c2(NP_037368) c3(ataxin-10 isoform 1) c4(175071, (75072) c5(175073, 175074, 175075, 175076, 175077, 175078, 175079, 175080, 175081, 175082, 175083, 175084) c6( ) c7(alP, ky, kB, ha, U, rf, vm, aan, aU, dr, wT, dR); #c1(ATXN1L) c2(NP_001131147) c3(ataxin-I-like) c4(52629) c5(52630, 52631, 52632, 52633, 52634, 52635, 52636) c6( ) c7(Kh, kB); #c1(ATXN2) c2(NP_002964) c3(ataxin-2) c4(134012, 134013) c5(134014, 134015, 134016, 134017, 134018, 134019, 134020, 134021, 134022, 134023, 134024, 134025, 134026, 134027, 134028) c6( ) c7(eR, alW, Ol, oe, e, yY, rf, Nt, lb, cz, cl, v, Kh, alS, bX, ez, GC, V, Kx, o, Gt, KB, aV, pN, WY, kf, aU, yZ, K, aU, an, bV, af, aU, cC, Ng, A, rU, alR, akL, eZ, aaE, EH, adE, hp, RI, t, nC, cm, alT, bb, ky, kB, c, Kv, U, nw, m, Kj, alV, act, X); #c1(ATXN2L) c2(NP_009176) c3(ataxin-2-like protein isoform A) c4(171310) c5(171311, 171312, 171313, 171314, 171315, 171316) c6( ) c7(fe); #c1(ATXN2L) c2(NP_663760) c3(ataxin-2-like protein isoform B) c4(241530) c5(241531, 241532, 241533, 241534, 241535, 241536) c6( ) c7(fe); #c1(ATXN2L) c2(NP_680780) c3(ataxin-2-like protein isoform C) c4(242332) c5(242333, 242334, 242335, 242336, 242337, 242338) c6( ) c7(fe); #c1(ATXN2L) c2(NP_680781) c3(ataxin-2-like protein isoform D) c4(242339) c5(242340, 242341, 242342, 242343, 242344, 242345) c6( ) c7(fe); #c1(ATXN2L) c2(NP_680782) c3(ataxin-2-like protein isoform E) c4(242346, 242347) c5(242348, 242349, 242350, 242351, 242352, 242353, 242354, 242355, 242356, 242357, 242358, 242359, 242360, 242361) c6( ) c7(fe); #c1(ATXN2L) c2(XP_006721071) c3(ataxin-2-like protein isoform X8) c4( ) c5(293839) c6( ) c7(fe); #c1(ATXN2L) c2(XP_011544024) c3(ataxin-2-like protein isoform X18) c4( ) c5(343192, 343193) c6( ) c7(fe); #c1(ATXN3) c2(NP_001121168) c3(ataxin-3 isoform ad) c4(45310, 45311) c5(45312, 45313, 45314, 45315, 45316, 45317, 45318, 45319, 45320, 45321, 45322, 45323) c6( ) c7(T, ob, yY, ht, rf, Nt, lb, aiC, GC, bS, Kh, ct, akg, PJ, fu, bX, ame, Kx, alZ, Na, lF, cF, amc, aV, HC, aU, alX, yZ, ama, K, hu, an, on, af, alY, aU, Ng, sq, xs, rU, TO, YG, EH, RI, Z, cm, nC, xw, dr, dR, rR, amd, ky, kB, c, se, ag, Kv, U, alV, Km, Kj, aan, amb, aen, ao); #c1(ATXN3) c2(NP_001158252) c3(ataxin-3 isoform u) c4( ) c5(63515, 63516, 63517, 63518, 63519) c6( ) c7(T, ob, yY, ht, rf, Nt, lb, aiC, GC, bS, Kh, ct, akg, PJ, fu, bX, ame, Kx, alZ, Na, lF, cF, amc, aV, HC, aU, alX, yZ, ama, K, hu, an, on, af, alY, aU, Ng, sq, xs, rU, TO, YG, EH, RI, Z, cm, nC, xw, dr, dR, rR, amd, ky, kB, c, se, ag, Kv, U, alV, Km, Kj, aan, amb, aen, ao); #c1(ATXN3) c2(NP_001158253) c3(ataxin-3 isoform y) c4(63520) c5(63521) c6( ) c7(T, ob, yY, ht, rf, Nt, lb, aiC, GC, bS, Kh, ct, akg, PJ, fu, bX, ame, Kx, alZ, Na, lF, cF, amc, aV, HC, aU, alX, yZ, ama, K, hu, an, on, af, alY, aU, Ng, sq, xs, rU, TO, YG, EH, RI, Z, cm, nC, xw, dr, dR, rR, amd, ky, kB, c, se, ag, Kv, U, alV, Km, Kj, aan, amb, aen, ao); #c1(ATXN3) c2(NP_004984) c3(ataxin-3 reference isoform) c4(151626, 151627, 151628, 151629, 151630, 151631, 151632, 151633, 151634, 151635) c5(151636, 151637, 151638, 151639, 151640, 151641, 151642, 151643, 151644, 151645, 151646, 151647, 151648, 151649, 151650, 151651, 151652, 151653, 151654, 151655, 151656, 151657, 151658, 151659, 151660, 151661, 151662, 151663, 151664, 151665, 151666, 151667, 151668, 151669, 151670, 151671, 151672, 151673, 151674, 151675, 151676, 151677, 151678, 151679, 151680, 151681, 151682, 151683, 151684, 151685, 151686, 151687, 151688, 151689, 151690, 151691, 151692, 151693, 151694, 151695) c6( ) c7(T, ob, yY, ht, rf, Nt, lb, aiC, GC, bS, Kh, ct, akg, PJ, fu, bX, ame, Kx, alZ, Na, lF, cF, amc, aV, HC, aU, alX, yZ, ama, K, hu, an, on, af, alY, aU, Ng, sq, xs, rU, TO, YG, EH, RI, Z, cm, nC, xw, dr, dR, rR, amd, ky, kB, c, se, ag, Kv, U, alV, Km, Kj, aan, amb, aen, ao); #c1(ATXN3) c2(NP_109376) c3(ataxin-3 isoform h) c4(219639) c5(219640, 219641, 219642, 219643, 219644, 219645) c6( ) c7(T, ob, yY, ht, rf, Nt, lb, aiC, GC, bS, Kh, ct, akg, PJ, fu, bX, ame, Kx, alZ, Na, lF, cF, amc, aV, HC, aU, alX, yZ, ama, K, hu, an, on, af, alY, alL, Ng, sq, xs, rU, TO, YG, EH, RI, Z, cm, nC, xw, dr, dR, rR, amd, ky, kB, c, se, ag, Kv, U, alV, Km, Kj, aan, amb, aen, ao); #c1(ATXN7) c2(NP_000324) c3 (ataxin-7 isoform a) c4(7121) c5(7122, 7123, 7124, 7125, 7126, 7127, 7128, 7129, 7130, 7131, 7132, 7133, 7134, 7135, 7136, 7137) c6( ) c7(yY, rf, Kh, C, bX, Sr, yZ, K, lQ, rU, EH, RI, XI, fq, nC, rR, amf, W, ky, kB, nb, Kv, U, nw, Pk); #c1(ATXN7) c2(NP_001121621) c3(ataxin-7 isoform c) c4(45885) c5(45886, 45887, 45888, 45889, 45890, 45891) c6( ) c7(yY, rf, Kh, C, bX, Sr, yZ, K, lQ, rU, EH, RI, XI, fq, nC, rR, amf, W, ky, kB, nb, Kv, U, nw, Pk); #c1(ATXN7) c2(NP_001170858) c3(ataxin-7 isoform b) c4(69547, 69548, 69549) c5(69550, 69551, 69552, 69553, 69554, 69555, 69556, 69557, 69558, 69559, 69560, 69561, 69562, 69563, 69564, 69565) c6( ) c7(yY, rf, Kh, C, bX, Sr, yZ, K, lQ, rU, EH, RI, XI, fq, nC, rR, amf, W, ky, kB, nb, Kv, U, nw, Pk); #c1(ATXN7L3B) c2(NP_001129734) c3(putative ataxin-7-like protein 3B) c4(52264) c5(52265, 52266, 52267, 52268, 52269, 52270) c6( ) c7(bA, pN); #c1(AUH) c2(NP_001689) c3(methylglutaconyl-CoA hydratase, mitochondrial precursor) c4(120001) c5(120002, 120003, 120004, 120005, 120006, 120007) c6( ) c7(dq, aw, my, eZ, EQ); #c1(AUH) c2(XP_005252129) c3(methylglutaconyl-CoA hydratase, mitochondrial isoform X7) c4(271560) c5(271561, 271562, 271563, 271564, 271565, 271566) c6( ) c7(dq, aw, my, eZ, EQ); #c1(AURKA) c2(XP_006723935) c3(aurora kinase A isoform X1) c4(295483, 295484, 295485, 295486, 295487, 295488, 295489) c5(295490, 295491, 295492, 295493, 295494, 295495, 295496, 295497, 295498, 295499, 295500, 295501, 295502, 295503, 295504, 295505, 295506, 295507, 295508, 295509, 295510, 295511, 295512, 295513, 295514, 295515, 295516, 295517, 295518, 295519, 295520, 295521, 295522, 295523, 295524, 295525, 295526, 295527, 295528, 295529, 295530, 295531, 295532, 295533, 295534, 295535) c6( ) c7(T, aC, pf, if, fd, bB, ci, x, aE, aeO, DO, K, hu, eL, aeR, aW, g, eF, cm, bw, eM, nB, cj, k, m, gn, wu, ht, n, fr, qN, zP, aje, br, ft, hw, gd, dl, ey, A1, aq, agF, kp, cM, dB, hk, at, it, B, fM, yk, aA, hy, bX, q, eA, qH, amg, lg, aT, ae, ne, jf, s, vT, iU, Y, r, en, cl, t, iK, ck, RW, gX, np, rA, bt, iW, jh, ad, av, hd, ib, iY, hP, wm, fb, d, BN, BO, qt, ff, eH, Ej, aF, iR, bz, es, bE, y, X); #c1(AURKA) c2(XP_011527323) c3(aurora kinase A isoform X2) c4( ) c5(321930, 321931, 321932, 321933, 321934, 321935, 321936, 321937, 321938, 321939, 321940, 321941, 321942, 321943, 321944, 321945, 321946, 321947, 321948, 321949, 321950, 321951, 321952, 321953) c6( ) c7(T, aC, pf, if, fd, bB, ci, x, aE, aeO, DO, K, hu, eL, aeR, aW, g, eF, cm, bw, eM, nB, cj, k, m, gn, wu, ht, n, fr, qN, zP, aje, br, ft, hw, gd, dL, ey, AI, aq, agF, kp, cM, dB, hk, at, it, B, fM, yk, aA, hy, bX, q, eA, qH, amg, lg, aT, ae, ne, jf, s, vT, iU, Y, r, en, cl, t, ik, ck, RW, gX, np, rA, bt, iW, jh, ad, av, hd, ib, iY, hP, wm, fb, d, BN, BO, qt, ff, eH, Ej, aF, iR, bz, es, bE, y, X); #c1(AURKB) c2(NP_001271455) c3(aurora kinase B isoform 3) c4(103974) c5(103975, 103976, 103977, 103978, 103979) c6( ) c7(T, ht, al, aA, bB, n, fd, ci, hF, pd, q, br, ad, eA, x, av, ft, hd, aE, gd, aT, ey, K, ae, dx, cp, d, iU, aW, Y, ff, r, agF, en, cl, g, t, cm, bz, bw, ck, gk, Dy, eM, dB, bE, gX, cj, hL, np, m, k, bt, it, B, vi); #c1(AURKC) c2(NP_001015878) c3(aurora kinase C isoform 1) c4(26198, 26199) c5(26200, 26201, 26202, 26203, 26204, 26205) c6( ) c7(T, ami, gU, if, aT, r, j, qN, amh, q); #c1(AURKC) c2(NP_001015879) c3(aurora kinase C isoform 2) c4(26206) c5(26207, 26208, 26209, 26210, 26211, 26212, 26213) c6( ) c7(T, ami, gU, if, aT, r, j, qN, amh, q); #c1(AURKC) c2(NP_003151) c3(aurora kinase C isoform 3) c4( ) c5(135725, 135726, 135727, 135728, 135729) c6( ) c7(T, ami, gU, if, aT, r, j, qN, amh, q); #c1(AUTS2) c2(NP_001120703) c3 (autism susceptibility gene 2 protein isoform 2) c4(44688) c5(44689, 44690, 44691, 44692, 44693, 44694) c6( ) c7(x, QK, oe, ha, amj, bK, k, nA, j, he, aR, bN); #c1(AUTS2) c2(NP_001120704) c3(autism susceptibility gene 2 protein isoform 3) c4(44695) c5(44696, 44697, 44698, 44699, 44700, 44701) c6( ) c7(x, QK, oe, ha, amj, bK, k, nA, j, he, aR, bN); #c1(AUTS2) c2(NP_056385) c3(autism susceptibility gene 2 protein isoform 1) c4(187378) c5(187379, 187380, 187381, 187382, 187383, 187384) c6( ) c7(x, QK, oe, ha, amj, bK, k, nA, j, he, aR, bN); #c1(AVEN) c2(NP_065104) c3(cell death regulator Aven) c4(203940) c5(203941, 203942, 203943, 203944, 203945, 203946, 203947) c6( ) c7(x, T, aC, ib, eM, iG, Ea, hf, hF); #c1(AVP) c2(NP_000481) c3(vasopressin-neurophysin 2-copeptin preproprotein) c4(9653) c5(9654, 9655, 9656, 9657, 9658, 9659) c6( ) c7(T, uk, e, al, bO, bB, Ja, alS, ci, amn, aE, M, adB, K, hu, aW, Fe, hl, g, hJ, cm, eM, Fh, Gs, amm, ht, amk, aj, ams, ct, ww, cd, ft, cc, amq, adN, Gc, adF, aml, aq, amo, hq, amt, cA, vL, E, it, B, adG, bL, amp, aA, cz, GC, j, il, q, J, qp, aT, cy, bK, an, rz, Fg, yj, qk, amw, amx, r, amr, wA, Rp, iK, FT, bN, amv, ac, amu, lw, rA, uG, Gd, bS, Sv, dG, Gn, kL, av, ot, oz, tT, adD, Gf, dq, aF, Z, bz, dr, gk, bE, dJ, pK, agZ); #c1(AVP11) c2(NP_068378) c3(arginine vasopressin-induced protein 1) c4(209373) c5(209374, 209375, 209376, 209377, 209378, 209379) c6( ) c7(De); #c1(AVPR1A) c2(NP_000697) c3(vasopressin V1a receptor) c4( ) c5(14614, 14615, 14616, 14617, 14618) c6( ) c7(bL, aw, EQ, cz, aj, v, ww, cd, J, ft, cc, bk, K, vP, VX, aml, gu, amr, qa, bN, hq, cO, xZ, E, Rm, mk, agZ); #c1(AVPR1A) c2(XP_005269059) c3(vasopressin V1a receptor isoform X1) c4(282268) c5(282269) c6( ) c7(bL, aw, EQ, cz, aj, v, ww, cd, J, ft, cc, bk, K, vP, VX, aml, gu, amr, qa, bN, hq, cO, xZ, E, Rm, mk, agZ); #c1(AVPR1B) c2(NP_000698) c3(vasopressin V1b receptor) c4(14619) c5(14620, 14621, 14622, 14623, 14624, 14625, 14626) c6( ) c7(ft, tb, cA, amy, r, vL, bS, bK, K, ct, ww, Z, q, bN, hq); #c1(AVPR2) c2(NP_001139623) c3(vasopressin V2 receptor isoform 2) c4(59559) c5(59560, 59561, 59562, 59563, 59564, 59565) c6( ) c7(AB, Ay, cz, n, amk, amC, nA, cd, amB, cc, adN, adD, adB, Nj, yj, amA, adF, Hu, dr, vU, amz, xb, adG); #c1(AVPR2) c2(XP_006724891) c3(vasopressin V2 receptor isoform X1) c4(296478) c5(296479, 296480, 296481, 296482, 296483, 296484, 296485, 296486, 296487, 296488, 296489, 296490, 296491) c6( ) c7(AB, Ay, cz, n, amk, amC, nA, cd, amB, cc, adN, adD, adB, Nj, yj, amA, adF, Hu, dr, vU, amz, xb, adG); #c1(AWAT1) c2(NP_001013597) c3(acyl-CoA wax alcohol acyltransferase 1) c4(25623) c5(25624, 25625, 25626, 25627, 25628, 25629) c6( ) c7(amD); #c1(AXDND1) c2(NP_653297) c3(axonemal dynein light chain domain-containing protein 1) c4(239523) c5(239524, 239525, 239526, 239527, 239528, 239529) c6( ) c7(V, cz); #c1(AXDND1) c2(XP_011507470) c3(axonemal dynein light chain domain-containing protein 1 isoform X1) c4( ) c5(297357) c6( ) c7(V, cz); #c1(AXDND1) c2(XP_011507472) c3(axonemal dynein light chain domain-containing protein 1 isoform X3) c4(297358, 297359, 297360) c5( ) c6( ) c7(V, cz); #c1(AXDND1) c2(XP_011507477) c3(axonemal dynein light chain domain-containing protein 1 isoform X6) c4(297361) c5(297362, 297363, 297364, 297365, 297366, 297367) c6( ) c7(V, cz); #c1(AXDND1) c2(XP_011507481) c3(axonemal dynein light chain domain-containing protein 1 isoform X10) c4(297368) c5( ) c6( ) c7(V, cz); #c1(AXDND1) c2(XP_011507482) c3(axonemal dynein light chain domain-containing protein 1 isoform X11) c4( ) c5(297369, 297370, 297371, 297372, 297373, 297374, 297375, 297376, 297377, 297378, 297379, 297380, 297381, 297382, 297383, 297384, 297385, 297386) c6( ) c7(V, cz); #c1(AXDND1) c2(XP_011507483) c3(axonemal dynein light chain domain-containing protein 1 isoform X12) c4( ) c5(297387, 297388, 297389, 297390, 297391) c6( ) c7(V, cz); #c1(AXIN1) c2(NP_003493) c3(axin-1 isoform a) c4(138763) c5(138764, 138765, 138766, 138767, 138768) c6( ) c7(T, al, aA, bB, ci, q, ad, av, amE, aT, K, an, ae, jf, aW, Y, ff, r, aq, aF, si, en, g, bz, amF, bF, gk, bE, gX, bD, B); #c1(AXIN1) c2(NP_851393) c3(axin-1 isoform b) c4(254569) c5(254570, 254571, 254572, 254573, 254574) c6( ) c7(T, al, aA, bB, ci, q, ad, av, amE, aT, K, an, ae, jf, aW, Y, ff, r, aq, aF, si, en, g, bz, amF, bF, gk, bE, gX, bD, B); #c1(AXIN1) c2(XP_011520984) c3(axin-1 isoform X1) c4( ) c5(315951) c6( ) c7(T, al, aA, bB, ci, q, ad, av, amE, aT, K, an, ae, jf, aW, Y, ff, r, aq, aF, si, en, g, bz, amF, bF, gk, bE, gX, bD, B); #c1(AXIN1) c2(XP_011520985) c3 (axin-1 isoform X2) c4( ) c5(315952, 315953) c6( ) c7(T, al, aA, bB, ci, q, ad, av, amE, aT, K, an, ae, jf, aW, Y, ff, r, aq, aF, si, en, g, bz, amF, bF, gk, bE, gX, bD, B); #c1(AXIN1) c2(XP_011520989) c3(axin-1 isoform X6) c4(315954) c5(315955, 315956, 315957, 315958, 315959) c6( ) c7(T, al, aA, bB, ci, q, ad, av, amE, aT, K, an, ae, jf, aW, Y, ff, r, aq, aF, si, en, g, bz, amF, bF, gk, bE, gX, bD, B); #c1(AXIN2) c2(XP_011523623) c3(axin-2 isoform X1) c4(318114) c5(318115, 318116, 318117, 318118, 318119, 318/20) c6( ) c7(T, aA, bB, fr, aml, ci, V, q, amH, Na, av, amJ, aT, Nc, dL, Qe, jf, A, B, s, aW, Y, r, aq, ahl, Z, t, amG, bz, cm, bF, iO, kP, eM, bE, gX, at, m, X); #c1(AXIN2) c2(XP_011523624) c3(axin-2 isoform X2) c4(318121, 318122) c5(318123, 318124, 318125, 318126, 318127, 318128, 318129, 318130, 318131, 318132, 318133, 318134) c6( ) c7(T, aA, bB, fr, aml, ci, V, q, amH, Na, av, amJ, aT, Nc, dL, Qe, jf, A, B, s, aW, Y, r, aq, ahl, Z, t, amG, bz, cm, bF, iO, kP, eM, bE, gX, at, m, X); #c1(AXL) c2(NP_001690) c3(tyrosine-protein kinase receptor UFO isoform 2 precursor) c4(120008) c5(120009, 120010, 120011, 120012, 120013, 120014) c6( ) c7(bM, T, dD, pf, ht, jh, eq, fr, cd, ad, eA, yn, dP, ib, aE, aT, dl, hu, fb, ae, eL, d, aW, Y, Ls, eH, AQ, kp, yq, g, mo, as, t, pt, bz, eF, Qt, iK, ck, kP, ac, eM, dB, hk, hc, iT, mx, ll, cS, UF, bt, B); #c1(AXL) c2(NP_068713) c3(tyrosine-protein kinase receptor UFO isoform 1 precursor) c4(210112) c5(210113, 210114, 210115, 210116, 210117, 210118) c6( ) c7(bM, T, dD, pf, ht, jh, eq, fr, cd, ad, eA, yn, dP, ib, aE, aT, dL, hu, fb, ae, eL, d, aW, Y, Ls, eH, AQ, kp, yq, g, mo, as, t, pt, bz, eF, Qt, iK, ck, kP, ac, eM, dB, hk, hc, iT, mx, ll, cS, UF, bt, B); #c1(AZGP1) c2(NP_001176) c3(zinc-alpha-2-glycoprotein precursor) c4(72458) c5(72459, 72460, 72461, 72462, 72463, 72464) c6( ) c7(T, al, aA, n, ko, v, ci, ad, av, iu, aE, aT, ae, UF, kl, JI, aW, kp, aF, g, f, bz, amK, hr, Uw, B); #c1(AZI2) c2(NP_001127904) c3(5-azacytidine-induced protein 2 isoform b) c4( ) c5(49464, 49465, 49466, 49467, 49468, 49469, 49470, 49471) c6( ) c7(iM, je, cr, n, tv, tu, C, af, amL); #c1(AZI2) c2(NP_001127905) c3(5-azacytidine-induced protein 2 isoform c) c4( ) c5(49472, 49473, 49474, 49475, 49476, 49477, 49478, 49479, 49480, 49481, 49482, 49483, 49484, 49485, 49486) c6( ) c7(iM, je, cr, n, tv, tu, C, af, amL); #c1(AZIZ) c2(NP_071906) c3(5-azacytidine-induced protein 2 isoform a) c4( ) c5(212219, 212220, 212221, 212222, 212223) c6( ) c7(iM, je, cr, n, tv, tu, C, af, aml); #c1(AZI2) c2(XP_005265450) c3(5-azacytidine-induced protein 2 isoform X4) c4( ) c5(279557) c6( ) c7(iM, je, cr, n, tv, tu, C, af, amL); #c1(AZI2) c2(XP_011532311) c3(5-azacytidine-induced protein 2 isoform X2) c4(328474) c5(328475) c6( ) c7(iM, je, cr, n, tv, tu, C, af, amL); #c1(AZI2) c2(XP_011532312) c3(5-azacytidine-induced protein 2 isoform X3) c4(328476, 328477) c5(328478, 328479) c6( ) c7(iM, je, cr, n, tv, tu, C, af, amL); #c1(AZI2) c2(XP_011532313) c3(5-azacytidine-induced protein 2 isoform X5) c4(328480, 328481) c5(328482, 328483, 328484) c6( ) c7(iM, je, cr, n, tv, tu, C, af, amL); #c1(AZIN1) c2(NP_001288597) c3(antizyme inhibitor 1 isoform 2) c4(113801) c5(113802, 113803, 113804, 113805, 113806, 113807, 113808, 113809, 113810, 113811, 113812, 113813) c6( ) c7(T, jx, ey, it, B, ad); #c1(AZIN1) c2(XP_011515429) c3(antizyme inhibitor 1 isoform X1) c4(308238, 308239) c5(308240, 308241, 308242, 308243, 308244, 308245, 308246, 308247, 308248, 308249) c6( ) c7(T, jx, ey, it, B, ad); #c1(AZIN2) c2(NP_001288754) c3(antizyme inhibitor 2 isoform 3) c4(113968) c5(113969, 113970, 113971, 113972, 113973, 113974, 113975, 113976, 113977, 113978) c6( ) c7(amN, av, Qy, d, eM, Tf, Y, ff, U, aT, cz, amM, m, en, g, as, t, bz); #c1(AZIN2) c2(XP_011538866) c3 (antizyme inhibitor 2 isoform X3) c4(336922, 336923) c5(336924, 336925, 336926, 336927, 336928, 336929, 336930, 336931, 336932, 336933, 336934, 336935, 336936, 336937, 336938, 336939) c6( ) c7(amN, av, Qy, d, eM, Tf, Y, ff, U, aT, cz, amM, m, en, g, as, t, bz); #c1(AZU1) c2(NP_001691) c3(azurocidin preproprotein) c4(120015, 120016, 120017, 120018, 120019, 120020, 120021, 120022, 120023, 120024, 120025, 120026, 120027, 120028, 120029, 120030, 120031, 120032, 120033, 120034, 120035, 120036, 120037, 120038, 120039, 120040, 120041, 120042, 120043, 120044, 120045, 120046, 120047, 120048, 120049, 120050, 120051, 120052, 120053, 120054, 120055, 120056, 120057, 120058, 120059, 120060, 120061, 120062, 120063, 120064, 120065, 120066, 120067, 120068, 120069, 120070, 120071, 120072, 120073, 120074, 120075, 120076, 120077) c5(120078, 120079, 120080, 120081, 120082, 120083, 120084, 120085, 120086, 120087, 120088, 120089) c6( ) c7(T, dc, oe, aT, h, ad); #c1(B2M) c2(NP_004039) c3(beta-2-microglobulin precursor) c4(143033, 143034) c5(143035, 143036, 143037, 143038, 143039, 143040, 143041, 143042, 143043, 143044, 143045, 143046, 143047, 143048, 143049, 143050, 143051, 143052, 143053, 143054, 143055, 143056, 143057, 143058, 143059, 143060, 143061) c6(143062) c7(bL, T, fM, dA, Ke, aD, amd, yG, jh, aA, bB, n, it, fd, fr, ci, aY, V, q, ad, eA, amR, dP, rw, ld, aT, h, yu, rB, K, ji, an, H, nJ, ae, amO, dx, mJ, A, je, s, iU, aW, Y, Zf, ff, eZ, ge, fl, r, CF, yy, Ej, uF, en, as, iV, YZ, cm, bz, ck, es, bF, amS, iM, abV, eM, iA, bE, eD, cj, gZ, E, amP, dy, Xa, R, Re, y, bt, WX, X, vi); #c1(B2M) c2(XP_006725245) c3(beta-2-microglobulin isoform X1) c4(296747) c5(296748, 296749, 296750, 296751, 296752, 296753, 296754, 296755, 296756, 296757, 296758, 296759, 296760, 296761) c6( ) c7(bL, T, fM, dA, Ke, aD, amd, yG, jh, aA, bB, n, it, fd, fr, ci, aY, V, q, ad, eA, amR, dP, rw, ld, aT, h, yu, rB, K, ji, an, H, nJ, ae, amO, dx, mJ, A, je, s, iU, aW, Y, Zf, ff, eZ, ge, fQ, r, CF, yy, Ej, uF, en, as, iV, YZ, cm, bz, ck, es, bF, amS, iM, abV, eM, iA, bE, eD, cj, gZ, E, amP, dy, Xa, R, Re, y, bt, WX, X, vi); #c1(B3GALNT1) c2(XP_005247921) c3(UDP-GalNAc:beta-1, 3-N-acetylgalactosaminyltransferase 1 isoform X1) c4(268079, 268080, 268081, 268082, 268083, 268084, 268085, 268086) c5(268087, 268088, 268089, 268090, 268091, 268092, 268093, 268094, 268095, 268096, 268097, 268098, 268099, 268100, 268101, 268102, 268103, 268104, 268105, 268106, 268107, 268108, 268109, 268110, 268111, 268112, 268113, 268114, 268115, 268116, 268117, 268118, 268119, 268120, 268121, 268122, 268123, 268124, 268125, 268126, 268127, 268128, 268129, 268130, 268131, 268132, 268133, 268134, 268135, 268136, 268137, 268138, 268139, 268140, 268141, 268142, 268143, 268144, 268145, 268146, 268147, 268148, 268149, 268150, 268151, 268152) c6( ) c7(amT, dB, aE, kp, Ro); #c1(B3GALNT1) c2(XP_011511564) c3(UDP-GalNAc:beta-1, 3-N-acetylgalactosaminyltransferase 1 isoform X2) c4( ) c5(302299, 302300) c6( ) c7(amT, dB, aE, kp, Ro); #c1(B3GALNT2) c2(NP_001264084) c3(UDP-GalNAc:beta-1, 3-N-acetylgalactosaminyltransferase 2 isoform 2 precursor) c4(97500, 97501) c5(97502, 97503, 97504, 97505, 97506, 97507, 97508, 97509, 97510, 97511, 97512) c6( ) c7(aW, FB, amU, aT); #c1(B3GALNT2) c2(NP_689703) c3(UDP-GalNAc:beta-1, 3-N-acetylgalactosaminyltransferase 2 isoform 1 precursor) c4(243616) c5(243617, 243618, 243619, 243620, 243621, 243622, 243623) c6( ) c7(aW, FB, amU, aT); #c1(B3GALT2) c2(NP_003774) c3(beta-1,3-galactosyltransferase 2) c4(140734) c5(140735, 140736, 140737, 140738, 140739, 140740) c6( ) c7(bA, an); #c1(B3GALT4) c2(NP_003773) c3(beta-1,3-galactosyltransferase 4) c4(140714) c5(140715, 140716, 140717, 140718, 140719, 140720, 140721, 140722, 140723, 140724, 140725, 140726, 140727, 140728, 140729, 140730, 140731, 140732, 140733) c6( ) c7(eZ); #c1(B3GALT5) c2(NP_149361) c3(beta-1,3-galactosyltransferase 5 isoform a) c4(227500) c5(227501, 227502, 227503, 227504, 227505, 227506, 227507, 227508, 227509, 227510, 227511, 227512, 227513, 227514) c6( ) c7(bE, T, bK, aE, g); #c1(B3GALT5) c2(NP_149362) c3(beta-1,3-galactosyltransferase 5 isoform b) c4( ) c5(227515, 227516, 227517, 227518) c6( ) c7(bE, T, bK, aE, g); #c1(B3GALTL) c2(NP_919299) c3(beta-1,3-glucosyltransferase precursor) c4(258486) c5(258487, 258488, 258489, 258490, 258491, 258492) c6( ) c7(W, HZ, amJ, amV, j, rz); #c1(B3GAT1) c2(XP_011541055) c3(galactosylgalactosylxylosylprotein 3-beta-glucuronosyltransferase 1 isoform X2) c4(339577) c5(339578, 339579, 339580, 339581, 339582, 339583, 339584, 339585, 339586) c6( ) c7(gV, T, bM, Dw, eR, Ke, DD, Or, aA, cz, fd, bS, j, ct, v, q, amW, ad, afb, x, ft, zw, ds, pX, SV, cU, h, amX, hn, K, lQ, amY, bc, dx, UM, hM, s, hG, Y, r, fe, gl, as, iV, Z, jT, zM, bz, cm, mK, iP, hq, Cv, ck, KT, acW, NL, Ji, mg, ME, k, y); #c1(B3GAT1) c2(XP_011541057) c3(galactosylgalactosylxylosylprotein 3-beta-glucuronosyltransferase 1 isoform X1) c4(339587) c5(339588, 339589, 339590, 339591, 339592, 339593, 339594, 339595, 339596, 339597, 339598, 339599) c6( ) c7(gV, T, bM, Dw, eR, Ke, DD, Or, aA, cz, fd, bS, j, ct, v, q, amW, ad, afb, x, ft, zw, ds, pX, SV, cu, h, amX, hn, K, lQ, amY, bc, dx, uM, hM, s, hG, Y, r, fe, gl, as, iV, Z, jT, zM, bz, cm, mk, iP, hq, Cv, ck, KT, acW, NL, Ji, mg, ME, k, y); #c1(B3GAT2) c2(NP_542780) c3(galactosylgalactosylxylosylprotein 3-beta-glucuronosyltransferase 2) c4(233049) c5(233050, 233051, 233052, 233053, 233054, 233055) c6( ) c7(qb); #c1(B3GAT3) c2(NP_001275652) c3(galactosylgalactosylxylosylprotein 3-beta-glucuronosyltransferase 3 isoform 4) c4(106906) c5(106907, 106908, 106909, 106910, 106911, 106912) c6( ) c7(LB, amF, fG, Az, bD, si, amZ); #c1(B3GAT3) c2(NP_036332) c3(galactosylgalactosylxylosylprotein 3-beta-glucuronosyltransferase 3 isoform 1) c4(173040) c5(173041, 173042, 173043, 173044, 173045, 173046) c6( ) c7(LB, amF, fG, Az, bD, si, amZ); #c1(B3GNT2) c2(XP_011530788) c3(N-acetyllactosaminide beta-1,3-N-acetylglucosaminyltransferase 2 isoform X1) c4(326839) c5(326840, 326841, 326842, 326843, 326844, 326845, 326846, 326847, 326848, 326849) c6( ) c7(aC, mR, fq, eZ, HD, y, ae, ad); #c1(B3GNT3) c2(XP_011525929) c3(N-acetyllactosaminide beta-1,3-N-acetylglucosaminyltransferase 3 isoform X1) c4(320335) c5(320336, 320337, 320338, 320339, 320340, 320341) c6( ) c7(cm, vj, ds, T, iW); #c1(B3GNT5) c2(XP_011511531) c3(lactosylceramide 1,3-N-acetyl-beta-D-glucosaminyltransferase isoform X1) c4(302181, 302182, 302183, 302184) c5(302185, 302186, 302187, 302188, 302189, 302190, 302191, 302192, 302193, 302194, 302195, 302196, 302197, 302198, 302199, 302200, 302201, 302202, 302203, 302204, 302205, 302206, 302207, 302208, 302209, 302210, 302211, 302212, 302213, 302214, 302215, 302216) c6( ) c7(d); #c1(B3GNT6) c2(NP_619651) c3(UDP-GlcNAc:betaGal beta-1,3-N-acetylglucosaminyltransferase 6) c4(237021, 237022, 237023, 237024) c5(237025, 237026, 237027, 237028, 237029, 237030, 237031, 237032, 237033, 237034, 237035, 237036, 237037, 237038, 237039, 237040, 237041, 237042, 237043, 237044, 237045, 237046, 237047, 237048, 237049) c6( ) c7(bF, T, aC, aE, s, bE, ci); #c1(B3GNT8) c2(NP_940942) c3(UDP-GlcNAc:betaGal beta-1,3-N-acetylglucosaminyltransferase 8 precursor) c4(260167) c5(260168, 260169, 260170, 260171, 260172, 260(73) c6( ) c7(bB, bE, X); #c1(B3GNTL1) c2(XP_006722335) c3(UDP-GlcNAc:betaGal beta-1,3-N-acetylglucosaminyltransferase-like protein 1 isoform X3) c4(294675) c5(294676, 294677, 294678, 294679, 294680, 294681) c6( ) c7(bB, bE); #c1(B4GALNT1) c2(NP_001263398) c3(beta-1,4 N-acetylgalactosaminyltransferase 1 isoform 3 precursor) c4( ) c5(97246, 97247, 97248, 97249, 97250, 97251, 97252, 97253, 97254, 97255, 97256, 97257) c6( ) c7(T, A, Y, gX, aT, cm, ci, ana, B, Qe); #c1(B4GALNT1) c2(NP_001469) c3(beta-1,4 N-acetylgalactosaminyltransferase 1 isoform 1 precursor) c4(117980) c5(117981, 117982, 117983, 117984, 117985, 117986) c6( ) c7(T, A, Y, gX, aT, cm, ci, ana, B, De); #c1(B4GALNT1) c2(XP_005268830) c3(beta-1,4 N-acetylgalactosaminyltransferase 1 isoform X2) c4(282110) c5( ) c6( ) c7(T, A, Y, gX, aT, cm, ci, ana, B, de); #c1(B4GALNT1) c2(XP_011536449) c3(beta-1,4 N-acetylgalactosaminyltransferase 1 isoform X1) c4(333814) c5( ) c6( ) c7(T, A, Y, gX, aT, cm, ci, ana, B, Qe); #c1(B4GALNT2) c2(NP_001152859) c3(beta-1,4 N-acetylgalactosaminyltransferase 2 isoform b) c4(60192) c5(60193, 60194, 60195, 60196, 60197, 60198) c6( ) c7(bL, T, dD, ZV, Zz, ea, e, aw, aA, sM, cz, vr, bS, j, ct, ci, eq, q, ad, av, ec, rS, aT, h, j, dQ, eN, hm, VC, bf, cW, aW, xs, Zt, r, uY, aF, Z, ck, ac, nB, wE, aka, vb, gX, hE, B); #c1(B4GALNT2) c2(NP_001152860) c3(beta-1,4 N-acetylgalactosaminyltransferase 2 isoform c) c4(60199) c5( ) c6( ) c7(bL, T, dD, ZV, Zz, ea, e, aw, aA, sM, cz, vr, bS, j, ct, ci, eq, q, ad, av, ec, rS, aT, h, j, dQ, eN, hm, VC, bf, cW, aW, xs, Zt, r, uY, aF, Z, ck, ac, nB, wE, aka, vb, gX, hE, B); #c1(B4GALNT2) c2(NP_703147) c3(beta-1,4 N-acetylgalactosaminyltransferase 2 isoform a) c4(245991) c5(245992, 245993, 245994, 245995, 245996, 245997, 245998, 245999, 246000, 246001, 246002, 246003) c6( ) c7(bL, T, dD, ZV, Zz, ea, e, aw, aA, sM, cz, vr, bS, j, ct, ci, eq, q, ad, av, ec, rS, aT, h, j, dQ, eN, hm, VC, bf, cW, aW, xs, Zt, r, uY, aF, Z, ck, ac, nB, wE, aka, vb, gX, hE, B); #c1(B4GALNT3) c2(NP_775864) c3(beta-1,4-N-acetylgalactosaminyltransferase 3) c4(250510) c5(250511, 250512, 250513, 250514, 250515, 250516) c6( ) c7(gZ); #c1(B4GALT1) c2(NP_001488) c3(beta-1,4-galactosyltransferase 1) c4(118136, 118137, 118138) c5(118139, 118140, 118141, 118142, 118143, 118144, 118145, 118146, 118147, 118148, 118149, 118150, 118151, 118152, 118153, 118154, 118155, 118156) c6( ) c7(ck, bM, T, ul, B, av, af, oj, r, vr, q, g, anb, anc); #c1(B4GALT1) c2(XP_005251497) c3(beta-1,4-galactosyltransferase 1 isoform X1) c4(270967) c5(270968, 270969, 270970, 270971, 270972, 270973) c6( ) c7(ck, bM, T, ul, B, av, af, oj, r, vr, q, g, anb, anc); #c1(B4GALT3) c2(NP_003770) c3(beta-1,4-galactosyltransferase 3) c4(140685, 140686, 140687, 140688, 140689) c5(140690, 140691, 140692, 140693, 140694, 140695, 140696, 140697, 140698, 140699, 140700, 140701, 140702, 140703, 140704, 140705, 140706, 140707, 140708, 140709, 140710, 140711, 140712, 140713) c6( ) c7(cm, q, r); #c1(B4GALT3) c2(XP_011508395) c3(beta-1,4-galactosyltransferase 3 isoform X2) c4( ) c5(299087, 299088, 299089, 299090, 299091, 299092, 299093, 299094, 299095, 299096, 299097) c6( ) c7(cm, q, r); #c1(B4GALT4) c2(NP_997708) c3(beta-1,4-galactosyltransferase 4) c4(265731, 265732, 265733, 265734, 265735) c5(265736, 265737, 265738, 265739, 265740, 265741, 265742, 265743, 265744, 265745, 265746, 265747, 265748, 265749, 265750, 265751, 265752, 265753, 265754) c6( ) c7(ad); #c1(B4GALT4) c2(XP_005247914) c3(beta-1,4-galactosyltransferase 4 isoform X2) c4(268053) c5(268054, 268055, 268056, 268057, 268058, 268059, 268060, 268061, 268062, 268063, 268064, 268065, 268066, 268067, 268068, 268069, 268070, 268071, 268072, 268073, 268074, 268075, 268076, 268077, 268078) c6( ) c7(ad); #c1(B4GALT5) c2(NP_004767) c3(beta-1,4-galactosyltransferase 5) c4(149438) c5(149439, 149440, 149441, 149442, 149443, 149444) c6( ) c7(ck, T, q); #c1(B4GALT6) c2(NP_004766) c3(beta-1,4-galactosyltransferase 6) c4(149417, 149418, 149419) c5(149420, 149421, 149422, 149423, 149424, 149425, 149426, 149427, 149428, 149429, 149430, 149431, 149432, 149433, 149434, 149435, 149436, 149437) c6( ) c7(c); #c1(B4GALT7) c2(NP_009186) c3(beta-1,4-galactosyltransferase 7) c4(171373) c5(171374, 171375, 171376, 171377, 171378, 171379, 171380, (71381, 171382) c6( ) c7(Az, uo, aq, K, Eh, B, and, anc); #c1(B4GALT7) c2(XP_011532723) c3(beta-1,4-galactosyltransferase 7 isoform X1) c4(328792) c5(328793, 328794, 328795, 328796, 328797) c6( ) c7(Az, uo, aq, K, Eh, B, and, anc); #c1(B4GAT1) c2(NP_006867) c3(beta-1,4-glucuronyltransferase 1) c4(168738, 168739) c5(168740, 168741, 168742, 168743, 168744, 168745, 168746, 168747, 168748, 168749, 168750, 168751) c6( ) c7(ane); #c1(B9DI) c2(NP_001230404) c3(B9 domain-containing protein 1 isoform c) c4(86281) c5(86282, 86283, 86284, 86285, 86286) c6( ) c7(anf, zG, ago); #c1(B9D1) c2(NP_056496) c3(B9 domain-containing protein 1 isoform b) c4(187775) c5(187776, 187777, 187778, 187779, 187780, 187781, 187782, 187783, 187784, 187785) c6( ) c7(anf, zG, ago); #c1(B9DI) c2(XP_005256664) c3(B9 domain-containing protein 1 isoform X4) c4(274267) c5(274268, 274269, 274270, 274271, 274272, 274273) c6( ) c7(anf, zG, ago); #c1(B9D1) c2(XP_005256666) c3(89 domain-containing protein 1 isoform X6) c4(274274) c5(274275, 274276, 274277, 274278, 274279, 274280) c6( ) c7(anf, zG, ago); #c1(B9D1) c2(XP_005256667) c3(B9 domain-containing protein 1 isoform X7) c4(274281) c5(274282, 274283, 274284, 274285, 274286, 274287) c6( ) c7(anf, zG, ago); #c1(B9D1) c2(XP_011522095) c3(B9 domain-containing protein 1 isoform X1) c4(316739) c5(316740, 316741, 316742, 316743, 316744, 316745) c6( ) c7(anf, zG, ago); #c1(B9DI) c2(XP_011522096) c3(B9 domain-containing protein 1 isoform X3) c4( ) c5(316746) c6( ) c7(anf, zG, ago); #c1(B902) c2(NP_085055) c3(B9 domain-containing protein 2) c4(219228) c5(219229, 219230, 219231, 219232, 219233, 219234) c6( ) c7(dy, ang, ago, dl, sj); #c1(BAALC) c2(NP_001019543) c3(brain and acute leukemia cytoplasmic protein isoform 2) c4( ) c5(27784, 27785, 27786, 27787, 27788, 27789) c6( ) c7(ck, x, T, GU, gm, hn, k, jj, hF, bt, er); #c1(BAALC) c2(NP_079088) c3(brain and acute leukemia cytoplasmic protein isoform 1) c4(216946, 216947, 216948) c5(216949, 216950, 216951, 216952, 216953, 216954, 216955, 216956, 216957, 216958, 216959, 216960) c6( ) c7(ck, x, T, GU, gm, hn, k, jj, hF, bt, er); #c1(BAAT) c2(NP_001692) c3(bile acid-CoA:amino acid N-acyltransferase) c4(120090) c5(120091, 120092, 120093, 120094, 120095, 120096, 120097, 120098, 120099, 120100) c6( ) c7(T, aA, bB, v, ci, C, q, cF, hd, aT, Yi, A, BO, s, aW, r, Ph, aF, cl, cl, nB, bE, bt, anh, ani); #c1(BABAM1) c2(NP_001275685) c3(BRISC and BRCA1-A complex member 1 isoform 1) c4(107047, 107048, 107049) c5(107050, 107051, 107052, 107053, 107054, 107055, 107056, 107057, 107058, 107059, 107060, 107061, 107062, 107063, 107064, 107065, 107066, 107067, 107068, 107069, 107070, 107071) c6( ) c7(aW, t, m, aT); #c1(BABAM1) c2(NP_001275686) c3(BRISC and BRCA1-A complex member 1 isoform 2) c4(107072) c5(107073, 107074, 107075, 107076, 107077, 107078) c6( ) c7(aW, t, m, aT); #c1(BACE1) c2(NP_001193978) c3(beta-secretase 1 isoform F) c4(83399) c5(83400, 83401, 83402, 83403, 83404, 83405, 83406, 83407, 83408, 83409, 83410) c6( ) c7(bL, e, ha, l, aj, uu, V, fK, J, anj, akN, sO, ds, K, H, af, ak, aaD, f, alj, cm, ek, acS, eD, dJ, U, oh, aS, R, xg, L); #c1(BACE1) c2(NP_036236) c3(beta-secretase 1 isoform A preproprotein) c4(172554, 172555) c5(172556, 172557, 172558, 172559, 172560, 172561, 172562) c6( ) c7(bL, e, ha, l, aj, uu, V, fK, J, anj, akN, sO, ds, K, H, af, ak, aaO, f, alj, cm, ek, acS, eD, dJ, U, oh, aS, R, xg, L); #c1(BACE1) c2(NP_620427) c3(beta-secretase 1 isoform C preproprotein) c4(237625) c5(237626, 237627, 237628, 237629, 237630, 237631) c6( ) c7(bL, e, ha, l, aj, uu, V, fK, J, anj, akN, sO, ds, K, H, af, ak, aaO, f, alj, cm, ek, acS, eD, dJ, U, oh, aS, R, xg, L); #c1(BACE1) c2(NP_620428) c3(beta-secretase 1 isoform B preproprotein) c4(237632) c5(237633, 237634, 237635, 237636, 237637, 237638) c6( ) c7(bL, e, ha, l, aj, uu, V, fK, J, anj, akN, sO, ds, K, H, af, ak, aaO, f, alj, cm, ek, acS, eD, dJ, U, oh, aS, R, xg, L); #c1(BACE1) c2(NP_620429) c3(beta-secretase 1 isoform D preproprotein) c4(237639) c5(237640, 237641, 237642, 237643, 237644, 237645) c6( ) c7(bL, e, ha, l, aj, uu, V, fK, J, anj, akN, sO, ds, K, H, af, ak, aaO, f, alj, cm, ek, acS, eD, dJ, U, oh, aS, R, xg, L); #c1(BACE2) c2(NP_036237) c3(beta-secretase 2 isoform A preproprotein) c4(172563, 172564) c5(172565, 172566, 172567, 172568, 172569, 172570, 172571, 172572, 172573, 172574, 172575, 172576) c6( ) c7(anj, T, hq, aW, aw, aT, FS, K, aaQ, f, alj, af, L); #c1(BACE2) c2(NP_620476) c3(beta-secretase 2 isoform C preproprotein) c4(237680) c5(237681, 237682, 237683, 237684, 237685, 237686) c6( ) c7(anj, T, hq, aW, aw, aT, FS, K, aaO, f, alj, af, L); #c1(BACE2) c2(NP_620477) c3(beta-secretase 2 isoform B preproprotein) c4(237687) c5(237688, 237689, 237690, 237691, 237692, 237693) c6( ) c7(anj, T, hq, aW, aw, aT, FS, K, aaO, f, alj, af, L); #c1 (BACH1) c2(NP_996749) c3(transcription regulator protein BACH1) c4(264424, 264425, 264426, 264427, 264428) c5(264429, 264430, 264431, 264432, 264433, 264434, 264435, 264436, 264437, 264438, 264439, 264440, 264441, 264442, 264443, 264444, 264445, 264446, 264447, 264448, 264449, 264450, 264451, 264452, 264453, 264454, 264455, 264456, 264457, 264458, 264459, 264460, 264461, 264462, 264463, 264464, 264465, 264466, 264467, 264468, 264469, 264470, 264471, 264472, 264473, 264474, 264475, 264476, 264477, 264478, 264479) c6( ) c7(T, os, aC, jx, n, oZ, F, aT, h, ji, K, af, aW, aq, zE, f, bz, t, fq, dc, m, bt, B); #c1(BACH2) c2(XP_005248815) c3(transcription regulator protein BACH2 isoform X2) c4(268594) c5(268595, 268596, 268597, 268598) c6( ) c7(bL, bM, T, aC, fM, EQ, aA, jh, n, cl, aj, j, qj, V, J, hD, h, hn, K, pk, A, iU, Of, Og, aF, hp, iV, t, cm, ck, ac, hr, cj, m, y, bt); #c1(BACH2) c2(XP_005248816) c3(transcription regulator protein BACH2 isoform X1) c4(268599) c5(268600, 268601, 268602, 268603, 268604, 268605, 268606, 268607, 268608, 268609, 268610) c6( ) c7(bL, bM, T, aC, fM, EQ, aA, jh, n, cl, aj, j, qj, V, J, hD, h, hn, K, pk, A, iU, Of, Og, aF, hp, iV, t, cm, ck, ac, hr, cj, m, y, bt); #c1(BACH2) c2(XP_011534344) c3(transcription regulator protein BACH2 isoform X3) c4( ) c5(330720, 330721) c6( ) c7(bL, bM, T, aC, fM, EQ, aA, jh, n, cl, aj, j, qj, V, J, hD, h, hn, K, pk, A, iU, Of, Og, aF, hp, iV, t, cm, ck, ac, hr, cj, m, y, bt); #c1(BACH2) c2(XP_011534345) c3(transcription regulator protein BACH2 isoform X4) c4( ) c5(330722, 330723, 330724, 330725, 330726, 330727, 330728, 330729) c6( ) c7(bL, bM, T, aC, fM, EQ, aA, jh, n, cl, aj, j, qj, V, J, hD, h, hn, K, pk, A, iU, Of, Og, aF, hp, iV, t, cm, ck, ac, hr, cj, m, y, bt); #c1 (BAD) c2(NP_004313) c3(bcl2 antagonist of cell death) c4(145081, 145082) c5(145083, 145084, 145085, 145086, 145087, 145088, 145089, 145090, 145091, 145092, 145093, 145094, 145095, 145096, 145097, 145098) c6( ) c7(T, fM, fR, fG, iW, eX, aA, bB, v, ci, q, cd, ad, eA, av, ft, Bc, aT, lG, cy, eV, K, bq, an, ae, eu, B, aW, Y, r, vs, g, cl, dw, t, cm, iP, ck, hL, wR, m, bt, X); #c1(BAG1) c2(NP_001165886) c3(BAG family molecular chaperone regulator 1 isoform BAG-IS) c4( ) c5(67920, 67921, 67922, 67923, 67924, 67925, 67926, 67927, 67928, 67929) c6( ) c7(T, anl, iH, aD, if, iW, aA, bB, n, hy, ci, qN, q, ad, eA, iu, tg, cF, aE, WY, ds, aT, lG, DO, bK, K, oi, ae, af, d, BN, B, s, qt, aW, ff, Rr, r, gl, aaE, aF, en, g, bz, ck, bP, c, eM, bE, ank, X, bt, ne); #c1(BAG1) c2(NP_004314) c3(BAG family molecular chaperone regulator 1 isoform BAG-IL) c4(145099, 145100, 145101) c5(145102, 145103, 145104, 145105, 145106, 145107, 145108, 145109) c6( ) c7(T, anl, iH, aD, if, iW, aA, bB, n, hy, ci, qN, q, ad, eA, iu, tg, cF, aE, WY, ds, aT, lG, DO, bK, K, oi, ae, af, d, BN, B, s, qt, aW, ff, Rr, r, gl, aaE, aF, en, g, bz, ck, bP, c, eM, bE, ank, X, bt, ne); #c1(BAG3) c2(NP_004272) c3(BAG family molecular chaperone regulator 3) c4(144757) c5(144758, 144759, 144760, 144761, 144762, 144763) c6( ) c7(T, aC, fR, iW, bB, hy, anm, fu, lu, cd, ad, hd, aE, oj, aT, h, lG, K, oi, ae, ul, d, BN, B, aW, ff, xs, wc, lo, en, mo, t, cm, hq, ck, kP, ac, nB, bE, mx, gX, bk, m, ano, bt, ann, ne); #c1(BAG3) c2(XP_005270344) c3(BAG family molecular chaperone regulator 3 isoform X1) c4(283355) c5(283356, 283357, 283358, 283359, 283360, 283361) c6( ) c7(T, aC, fR, iW, bB, hy, anm, fu, lu, cd, ad, hd, aE, oj, aT, h, lG, K, oi, ae, ul, d, BN, B, aW, ff, xs, wc, lo, en, mo, t, cm, hq, ck, kP, ac, nB, bE, mx, gX, bk, m, ano, bt, ann, ne); #c1(BAG4) c2(NP_001191807) c3(BAG family molecular chaperone regulator 4 isoform 2) c4(81834) c5(81835, 81836, 81837, 81838, 81839, 81840) c6( ) c7(T, ci, iW); #c1(BAG4) c2(NP_004865) c3(BAG family molecular chaperone regulator 4 isoform 1) c4(150428) c5(150429, 150430, 150431, 150432, 150433, 150434) c6( ) c7(T, ci, iW); #c1(BAG5) c2(NP_001015049) c3(BAG family molecular chaperone regulator 5 isoform a) c4(26142) c5(26143, 26144, 26145, 26146, 26147, 26148, 26149, 26150, 26151) c6( ) c7(BN, iW, hy, K, ae, ne, ad); #c1(BAG5) c2(NP_004864) c3(BAG family molecular chaperone regulator 5 isoform b) c4(150413, 150414) c5(150415, 150416, 150417, 150418, 150419, 150420, 150421, 150422, 150423, 150424, 150425, 150426, 150427) c6( ) c7(BN, iW, hy, K, ae, ne, ad); #c1(BAG6) c2(NP_001186626) c3(large proline-rich protein BAG6 isoform c) c4(78003, 78004) c5(78005, 78006, 78007, 78008, 78009, 78010, 78011, 78012, 78013, 78014, 78015, 78016, 78017, 78018, 78019, 78020, 78021, 78022, 78023, 78024, 78025, 78026, 78027, 78028, 78029, 78030, 78031, 78032, 78033, 78034, 78035, 78036, 78037, 78038, 78039, 78040, 78041, 78042, 78043, 78044, 78045, 78046, 78047, 78048, 78049, 78050, 78051, 78052, 78053, 78054, 78055, 78056, 78057, 78058, 78059, 78060, 78061, 78062, 78063, 78064, 78065, 78066, 78067, 78068, 78069, 78070, 78071, 78072, 78073, 78074, 78075, 78076, 78077, 78078, 78079, 78080, 78081, 78082, 78083, 78084, 78085, 78086, 78087, 78088, 78089, 78090, 78091, 78092, 78093, 78094, 78095, 78096, 78097, 78098, 78099, 78100, 78101, 78102, 78103, 78104, 78105, 78106, 78107, 78108, 78109, 78110, 78111, 78112, 78113, 78114, 78115, 78116, 78117, 78118, 78119, 78120, 78121, 78122, 78123, 78124, 78125, 78126, 78127, 78128, 78129, 78130, 78131, 78132, 78133, 78134, 78135, 78136, 78137, 78138, 78139, 78140, 78141, 78142, 78143, 78144, 78145, 78146, 78147, 78148, 78149, 78150, 78151, 78152, 78153, 78154, 78155, 78156, 78157, 78158, 78159, 78160, 78161, 78162, 78163, 78164, 78165, 78166, 78167, 78168, 78169, 78170, 78171, 78172, 78173, 78174, 78175, 78176, 78177, 78178, 78179, 78180, 78181, 78182, 78183, 78184, 78185, 78186, 78187, 78188, 78189, 78190, 78191, 78192, 78193, 78194, 78195, 78196, 78197, 78198, 78199, 78200, 78201) c6( ) c7(T, iQ, od, eF, ht, cE, eZ, cj, fd, pD, k, C, g, bD, lf, bz, iu, y); #c1 (BAG6) c2(NP_00186627) c3(large proline-rich protein BAG6 isoform d) c4(78202) c5(78203, 78204, 78205, 78206, 78207, 78208, 78209, 78210, 78211, 78212, 78213, 78214, 78215, 78216, 78217, 78218, 78219, 78220, 78221, 78222, 78223, 78224, 78225, 78226, 78227, 78228, 78229, 78230, 78231, 78232, 78233, 78234, 78235, 78236, 78237, 78238, 78239, 78240, 78241, 78242, 78243, 78244, 78245, 78246, 78247, 78248, 78249, 78250, 78251, 78252, 78253, 78254, 78255, 78256, 78257, 78258, 78259, 78260, 78261, 78262, 78263, 78264, 78265, 78266, 78267, 78268, 78269, 78270, 78271, 78272, 78273, 78274, 78275, 78276, 78277, 78278, 78279, 78280, 78281, 78282, 78283, 78284) c6( ) c7(T, iQ, od, eF, ht, cE, eZ, cj, fd, pD, k, C, g, bD, lf, bz, iu, y); #c1(BAG6) c2(NP_004630) c3(large proline-rich protein BAG6 isoform a) c4(148296, 148297, 148298) c5(148299, 148300, 148301, 148302, 148303, 148304, 148305, 148306, 148307, 148308, 148309, 148310, 148311, 148312, 148313, 148314, 148315, 148316, 148317, 148318, 148319, 148320, 148321, 148322, 148323, 148324, 148325, 148326, 148327, 148328, 148329, 148330, 148331, 148332, 148333, 148334, 148335, 148336, 148337, 148338, 148339, 148340, 148341, 148342, 148343, 148344, 148345, 148346, 148347, 148348, 148349, 148350) c6( ) c7(T, iQ, od, eF, ht, cE, eZ, cj, fd, pD, k, C, g, bD, lf, bz, iu, y); #c1(BAG6) c2(NP_542433) c3 (large proline-rich protein BAG6 isoform b) c4(232877, 232878, 232879, 232880, 232881, 232882, 232883) c5(232884, 232885, 232886, 232887, 232888, 232889, 232890, 232891, 232892, 232893, 232894, 232895, 232896, 232897, 232898, 232899, 232900, 232901, 232902, 232903, 232904, 232905, 232906, 232907, 232908, 232909, 232910, 232911, 232912, 232913, 232914, 232915, 232916, 232917, 232918, 232919, 232920, 232921, 232922, 232923, 232924, 232925, 232926, 232927, 232928, 232929, 232930, 232931, 232932, 232933, 232934, 232935, 232936, 232937, 232938, 232939) c6( ) c7(T, iQ, od, eF, ht, cE, eZ, cj, fd, pD, k, C, g, bD, lf, bz, iu, y); #c1(BAG6) c2(XP_006715255) c3(large proline-rich protein BAG6 isoform X6) c4(289090, 289091, 289092) c5(289093, 289094, 289095, 289096, 289097, 289098, 289099, 289100, 289101, 289102, 289103, 289104, 289105, 289106, 289107, 289108, 289109, 289110, 289111, 289112, 289113, 289114, 289115, 289116, 289117, 289118, 289119, 289120) c6( ) c7(T, iQ, od, eF, ht, cE, eZ, cj, fd, pD, k, C, g, bD, lf, bz, iu, y); #c1(BAG6) c2(XP_006715264) c3(large proline-rich protein BAG6 isoform X15) c4( ) c5(289121, 289122, 289123, 289124, 289125, 289126, 289127, 289128, 289129, 289130, 289131, 289132, 289133, 289134, 289135, 289136, 289137, 289138, 289139, 289140, 289141, 289142, 289143, 289144, 289145, 289146) c6( ) c7(T, iQ, od, eF, ht, cE, eZ, cj, fd, pD, k, C, g, bD, lf, bz, iu, y); #c1(BAG6) c2(XP_006725986) c3(large proline-rich protein BAG6 isoform X6) c4(297037, 297038) c5( ) c6( ) c7(T, iQ, od, eF, ht, cE, eZ, cj, fd, pD, k, C, g, bD, lf, bz, iu, y); #c1(BAG6) c2(XP_011513199) c3(large proline-rich protein BAG6 isoform X4) c4(305231) c5( ) c6( ) c7(T, iQ, od, eF, ht, cE, eZ, cj, fd, pD, k, C, g, bD, lf, bz, iu, y); #c1(BAG6) c2(XP_011513202) c3(large proline-rich protein BAG6 isoform X7) c4(305232) c5( ) c6( ) c7(T, iQ, od, eF, ht, cE, eZ, cj, fd, pD, k, C, g, bD, lf, bz, iu, y); #c1(BAG6) c2(XP_011513203) c3(large proline-rich protein BAG6 isoform X8) c4(305233, 305234) c5( ) c6( ) c7(T, iQ, od, eF, ht, cE, eZ, cj, fd, pD, k, C, g, bD, lf, bz, iu, y); #c1(BAG6) c2(XP_011513205) c3 (large proline-rich protein BAG6 isoform X11) c4(305235) c5( ) c6( ) c7(T, iQ, od, eF, ht, cE, eZ, cj, fd, pD, k, C, g, bD, lf, bz, iu, y); #c1(BAG6) c2(XP_011513207) c3(large proline-rich protein BAG6 isoform X13) c4(305236, 305237) c5(305238, 305239, 305240, 305241, 305242, 305243, 305244, 305245, 305246, 305247, 305248, 305249, 305250, 305251, 305252, 305253, 305254, 305255, 305256, 305257, 305258, 305259, 305260, 305261) c6( ) c7(T, iQ, od, eF, ht, cE, eZ, cj, fd, pD, k, C, g, bD, lf, bz, iu, y); #c1(BAG6) c2(XP_011513209) c3(large proline-rich protein BAG6 isoform X16) c4(305262) c5(305263, 305264, 305265, 305266, 305267, 305268, 305269, 305270, 305271, 305272, 305273, 305274, 305275, 305276, 305277, 305278, 305279, 305280, 305281, 305282, 305283, 305284, 305285, 305286, 305287, 305288, 305289, 305290, 305291, 305292, 305293, 305294, 305295, 305296, 305297, 305298, 305299, 305300, 305301, 305302, 305303, 305304, 305305, 305306, 305307, 305308, 305309, 305310, 305311, 305312, 305313, 305314, 305315, 305316, 305317, 305318) c6( ) c7(T, iQ, od, eF, ht, cE, eZ, cj, fd, pD, k, C, g, bD, lf, bz, iu, y); #c1(BAG6) c2(XP_011513210) c3(large proline-rich protein BAG6 isoform X17) c4( ) c5(305319, 305320, 305321, 305322, 305323, 305324, 305325, 305326, 305327, 305328, 305329, 305330, 305331, 305332, 305333, 305334, 305335, 305336, 305337, 305338, 305339, 305340, 305341, 305342, 305343, 305344, 305345, 305346, 305347, 305348, 305349, 305350, 305351, 305352, 305353, 305354, 305355, 305356, 305357, 305358, 305359, 305360, 305361, 305362, 305363, 305364, 305365, 305366, 305367, 305368, 305369, 305370, 305371, 305372, 305373, 305374, 305375, 305376, 305377, 305378, 305379, 305380, 305381, 305382, 305383, 305384, 305385, 305386, 305387, 305388, 305389, 305390, 305391, 305392, 305393, 305394, 305395, 305396, 305397, 305398, 305399, 305400, 305401, 305402, 305403, 305404, 305405, 305406, 305407, 305408, 305409, 305410, 305411, 305412, 305413, 305414, 305415, 305416, 305417, 305418, 305419, 305420, 305421, 305422, 305423, 305424, 305425, 305426, 305427, 305428, 305429, 305430, 305431, 305432, 305433, 305434, 305435, 305436) c6( ) c7(T, iQ, od, eF, ht, cE, eZ, cj, fd, pD, k, C, g, bD, lf, bz, iu, y); #c1(BAG6) c2(XP_011546389) c3(large proline-rich protein BAG6 isoform X5) c4(345168) c5( ) c6( ) c7(T, iQ, od, eF, ht, cE, eZ, cj, fd, pD, k, C, g, bD, lf, bz, iu, y); #c1(BAG6) c2(XP_011546397) c3(large proline-rich protein BAG6 isoform X14) c4(345169) c5( ) c6( ) c7(T, iQ, od, eF, ht, cE, eZ, cj, fd, pD, k, C, g, bD, lf, bz, iu, y); #c1(BAIAPZL1) c2(NP_061330) c3(brain-specific angiogenesis inhibitor 1-associated protein 2-like protein 1) c4(200043) c5(200044, 200045, 200046, 200047, 200048, 200049, 200050, 200051) c6( ) c7(y, ad); #c1(BAIAP3) c2(NP_001186025) c3(BAI1-associated protein 3 isoform 2) c4(76887) c5(76888, 76889, 76890, 76891, 76892, 76893, 76894, 76895, 76896, 76897, 76898, 76899) c6( ) c7(anp, T, ac, hq); #c1(BAIAP3) c2(NP_001186026) c3(BAI1-associated protein 3 isoform 3) c4(76900, 76901) c5( ) c6( ) c7(anp, T, ac, hq); #c1(BAIAP3) c2(NP_003924) c3(BAI1-associated protein 3 isoform 1) c4(142212) c5(142213, 142214, 142215, 142216, 142217, 142218, 142219, 142220, 142221, 142222, 142223, 142224) c6( ) c7(anp, T, ac, hq); #c1(BAIAP3) c2(XP_011521030) c3(BAI1-associated protein 3 isoform X1) c4(315973) c5(315974, 315975, 315976, 315977, 315978, 315979, 315980, 315981, 315982, 315983) c6( ) c7(anp, T, ac, hq); #c1(BAK1) c2(NP_001179) c3(bcl-2 homologous antagonist/killer) c4(73317, 73318, 73319) c5(73320, 73321, 73322, 73323, 73324, 73325, 73326, 73327, 73328, 73329, 73330, 73331, 73332, 73333, 73334, 73335, 73336, 73337, 73338, 73339, 73340, 73341) c6( ) c7(T, if, iW, aA, ci, bX, qN, eA, av, ib, HC, KH, aT, hD, iv, dL, cp, je, bf, s, vT, aW, Y, ff, r, fQ, aF, hp, Hu, en, bu, dw, bz, Ld, op, iM, eM, nB, vA, gX, cj, at, bm, m, y, X); #c1(BAMBI) c2(NP_036474) c3(BMP and activin membrane-bound inhibitor homolog precursor) c4(174126, 174127) c5(174128, 174129, 174130, 174131, 174132, 174133, 174134, 174135, 174136, 174137, 174138, 174139) c6( ) c7(T, B, NE, bE, aW, fc, r, aT, bB, aq, dL, v, ci, cm, eL, q); #c1(BANF1) c2(NP_001137457) c3(barrier-to-autointegration factor) c4(55807, 55808) c5(55809, 55810, 55811, 55812, 55813, 55814, 55815, 55816, 55817, 55818, 55819, 55820, 55821, 55822, 55823, 55824, 55825, 55826, 55827, 55828, 55829, 55830, 55831, 55832) c6( ) c7(T, anr, xs, anq, La, nA, agk, bt); #c1(BANK1) c2(NP_001077376) c3(B-cell scaffold protein with ankyrin repeats isoform 2) c4(37775) c5(37776, 37777, 37778, 37779, 37780) c6( ) c7(ans, ai, pb, OV, mj, A, yW, eZ, ez, lR, fl, an, y, lM, Pr, mC, Pm); #c1(BANK1) c2(NP_001120979) c3(B-cell scaffold protein with ankyrin repeats isoform 3) c4(45038) c5(45039, 45040, 45041, 45042, 45043, 45044, 45045, 45046, 45047, 45048) c6( ) c7(ans, ai, pb, OV, mj, A, yW, eZ, ez, lR, fl, an, y, lM, Pr, mC, Pm); #c1(BANK1) c2(NP_060405) c3(B-cell scaffold protein with ankyrin repeats isoform 1) c4(195829, 195830) c5(195831, 195832, 195833, 195834, 195835, 195836, 195837, 195838, 195839, 195840, 195841, 195842, 195843) c6( ) c7(ans, ai, pb, OV, mj, A, yW, eZ, ez, lR, fl, an, y, IM, Pr, mC, Pm); #c1(BANP) c2(NP_001167010) c3(protein BANP isoform c) c4(68821, 68822, 68823, 68824) c5(68825, 68826, 68827, 68828, 68829, 68830) c6( ) c7(ant, T, ac, aW, aT, la, ab, h, gv, K); #c1(BANP) c2(NP_001167011) c3(protein BANP isoform d) c4(68831, 68832) c5(68833, 68834, 68835, 68836, 68837, 68838, 68839, 68840, 68841, 68842, 68843, 68844) c6( ) c7(ant, T, ac, aW, aT, la, ab, h, gv, K); #c1(BANP) c2(NP_001167012) c3(protein BANP isoform e) c4(68845) c5(68846, 68847, 68848, 68849, 68850, 68851, 68852, 68853, 68854, 68855, 68856, 68857, 68858, 68859, 68860, 68861, 68862, 68863, 68864, 68865, 68866, 68867, 68868, 68869) c6( ) c7(ant, T, ac, aW, aT, la, ab, h, gv, K); #c1(BANP) c2(NP_001167014) c3(protein BANP isoform g) c4(68870) c5(68871, 68872, 68873, 68874, 68875, 68876, 68877, 68878, 68879, 68880) c6( ) c7(ant, T, ac, aW, aT, la, ab, h, gv, K); #c1(BANP) c2(NP_060339) c3(protein BANP isoform a) c4(195473, 195474) c5(195475, 195476, 195477, 195478, 195479, 195480, 195481, 195482, 195483, 195484, 195485, 195486, 195487) c6( ) c7(ant, T, ac, aW, aT, la, ab, h, gv, K); #c1(BANP) c2(NP_524576) c3(protein BANP isoform b) c4(231153, 231154, 231155) c5(231156, 231157, 231158, 231159, 231160, 231161, 231162, 231163, 231164, 231165) c6( ) c7(ant, T, ac, aW, aT, la, ab, h, gv, K); #c1(BANP) c2(XP_005256066) c3(protein BANP isoform X1) c4(273930) c5(273931, 273932, 273933, 273934, 273935, 273936) c6( ) c7(ant, T, ac, aW, aT, la, ab, h, gv, K); #c1(BANP) c2(XP_011521482) c3(protein BANP isoform X4) c4( ) c5(316229, 316230, 316231, 316232, 316233) c6( ) c7(ant, T, ac, aW, aT, la, ab, h, gv, K); #c1(BANP) c2(XP_011521488) c3(protein BANP isoform X11) c4( ) c5(316234, 316235, 316236, 316237, 316238, 316239) c6( ) c7(ant, T, ac, aW, aT, la, ab, h, gv, K); #c1(BAPI) c2(NP_004647) c3(ubiquitin carboxyl-terminal hydrolase BAP1) c4(148488, 148489) c5(148490, 148491, 148492, 148493, 148494, 148495, 148496, 148497, 148498, 148499, 148500, 148501) c6( ) c7(sc, Mb, T, dA, aA, n, fr, pd, anu, ip, jz, ib, py, Nn, aT, K, anw, anv, nO, aW, Y, aF, t, bw, bz, XT, iP, es, Ew, Dy, iA, hk, go, m, anx, y, VB, hS); #c1(BARD1) c2(NP_000456) c3(BRCA1-associated RING domain protein 1 isoform 1) c4(9249) c5(9250, 9251, 9252, 9253, 9254, 9255) c6( ) c7(T, if, bB, j, ci, qN, lu, hw, aT, dL, K, qw, aW, cl, cm, t, bz, eM, bE, at, m, X); #c1(BARD1) c2(NP_001269472) c3(BRCA1-associated RING domain protein 1 isoform 2) c4(101734) c5( ) c6( ) c7(T, if, bB, j, ci, qN, lu, hw, aT, dL, K, qw, aW, cl, cm, t, bz, eM, bE, at, m, X); #c1(BARD1) c2(XP_011509869) c3(BRCA1-associated RING domain protein 1 isoform X1) c4( ) c5(300473, 300474, 300475, 300476, 300477, 300478) c6( ) c7(T, if, bB, j, ci, qN, lu, hw, aT, dL, K, qw, aW, cl, cm, t, bz, eM, bE, at, m, X); #c1(BARHL1) c2(NP_064448) c3(barH-like 1 homeobox protein) c4(202514) c5(202515, 202516, 202517, 202518, 202519, 202520, 202521, 202522, 202523, 202524, 202525, 202526) c6( ) c7(jf); #c1(BARX1) c2(NP_067545) c3(homeobox protein BarH-like 1) c4(208988, 208989) c5(208990, 208991, 208992, 208993, 208994, 208995, 208996, 208997, 208998, 208999, 209000, 209001, 209002) c6( ) c7(Nc, any, ac, pf); #c1(BARX2) c2(NP_003649) c3(homeobox protein BarH-like 2) c4(139924) c5(139925, 139926, 139927, 139928, 139929, 139930, 139931) c6( ) c7(anA, aW, aT, lm, anz, m, g, t); #c1(BASP1) c2(NP_006308) c3(brain acid soluble protein 1) c4(163871, 163872) c5(163873, 163874, 163875, 163876, 163877, 163878, 163879, 163880, 163881, 163882, 163883, 163884) c6( ) c7(anB, zQ, iT, ci, zP, B); #c1(BATF) c2(NP_006390) c3(basic leucine zipper transcriptional factor ATF-like) c4(164620) c5(164621, 164622, 164623, 164624, 164625, 164626, 164627) c6( ) c7(h, V, fq, z, A); #c1(BATF2) c2(NP_612465) c3(basic leucine zipper transcriptional factor ATF-like 2 isoform 1) c4(236585) c5(236586, 236587, 236588, 236589, 236590, 236591) c6( ) c7(ck, iu, T, Y, ds, cU, ci, B); #c1(BAX) c2(NP_001278357) c3(apoptosis regulator BAX isoform 1) c4( ) c5(109598, 109599, 109600, 109601, 109602, 109603) c6( ) c7(bM, G, T, aC, fR, if, al, bB, ci, x, Vp, aE, il, Bc, pX, ajv, K, pk, dF, eL, aiJ, cp, de, eG, aW, az, g, eF, eM, nB, AM, iT, vA, U, cj, k, m, wu, zX, aw, yG, ht, n, aj, fr, qN, JW, eW, ft, no, jz, gd, h, lG, eV, hn, dl, ox, on, pl, afu, wC, Lh, aq, Hu, zZ, op, iO, bP, anC, hL, hR, E, at, it, B, fM, eX, aA, cz, hy, bX, V, q, J, eA, HC, aT, cy, rO, jj, bq, an, ae, af, ne, kl, s, iU, Y, r, en, cl, t, iK, ck, sS, iQ, hc, anD, fD, im, bt, LS, id, qb, iW, jh, dG, Fb, ad, av, hd, hD, wm, uD, d, BN, BO, ff, eH, kD, aF, iV, iR, bz, bF, bE, gZ, bC, y, X); #c1(BAX) c2(NP_004315) c3(apoptosis regulator BAX isoform beta) c4(145110) c5(145111, 145112, 145113, 145114, 145115, 145116) c6( ) c7(bM, G, T, aC, fR, if, al, bB, ci, x, Vp, aE, il, Bc, pX, ajv, K, pk, dF, eL, aiJ, cp, Qe, eG, aW, az, g, eF, eM, nB, AM, iT, vA, U, cj, k, m, wu, zX, aw, yG, ht, n, aj, fr, qN, JW, eW, ft, no, jz, gd, h, lG, eV, hn, dL, ox, on, pl, afu, wC, Lh, aq, Hu, zZ, op, iO, bP, anC, hL, hR, E, at, it, B, fM, eX, aA, cz, hy, bX, V, q, J, eA, HC, aT, cy, rO, jj, bq, an, ae, af, ne, kl, s, iU, Y, r, en, cl, t, ik, ck, sS, iQ, hc, anD, fD, im, bt, LS, id, qb, iW, jh, dG, Fb, ad, av, hd, hD, wm, uD, d, BN, BO, ff, eH, kD, aF, iV, iR, bz, bF, bE, gZ, bC, y, X); #c1(BAX) c2(NP_620116) c3(apoptosis regulator BAX isoform alpha) c4(237287) c5(237288, 237289, 237290, 237291, 237292, 237293, 237294) c6( ) c7(bM, G, T, aC, fR, if, al, bB, ci, x, Vp, aE, il, Bc, pX, ajv, K, pk, dF, eL, aiJ, cp, Qe, eG, aW, az, g, eF, eM, nB, AM, iT, vA, U, cj, k, m, wu, zX, aw, yG, ht, n, aj, fr, qN, JW, eW, ft, no, jz, gd, h, lG, eV, hn, dl, ox, on, pl, afu, wC, Lh, aq, Hu, zZ, op, iO, bP, anC, hL, hR, E, at, it, B, fM, eX, aA, cz, hy, bX, V, q, J, eA, HC, aT, cy, rO, jj, bq, an, ae, af, ne, kl, s, iU, Y, r, en, cl, t, ik, ck, sS, iQ, hc, anD, fD, im, bt, LS, id, qb, iW, jh, dG, Fb, ad, av, hd, hD, wm, uD, d, BN, BO, ff, eH, KD, aF, iV, iR, bz, bF, bE, gZ, bC, y, X); #c1(BAX) c2(NP_620118) c3(apoptosis regulator BAX isoform delta) c4(237295) c5(237296, 237297, 237298, 237299, 237300, 237301) c6( ) c7(bM, G, T, aC, fR, if, al, bB, ci, x, Vp, aE, il, Bc, pX, ajv, K, pk, dF, eL, aiJ, cp, Qe, eG, aW, az, g, eF, eM, nB, AM, iT, vA, U, cj, k, m, wu, zX, aw, yG, ht, n, aj, fr, qN, JW, eW, ft, no, jz, gd, h, lG, eV, hn, dL, ox, on, pl, afu, wC, Lh, aq, Hu, zZ, op, iO, bP, anC, hL, hR, E, at, it, B, fM, eX, aA, cz, hy, bX, V, q, J, eA, HC, aT, cy, rO, jj, bq, an, ae, af, ne, kl, s, iU, Y, r, en, cl, t, ik, ck, sS, iQ, hc, anD, fD, im, bt, LS, id, qb, iW, jh, dG, Fb, ad, av, hd, hD, wm, uD, d, BN, BO, ff, eH, kD, aF, iV, iR, bz, bF, bE, gZ, bC, y, X); #c1(BAX) c2(NP_620119) c3(apoptosis regulator BAX isoform sigma) c4(237302, 237303) c5(237304, 237305, 237306, 237307, 237308, 237309, 237310, 237311, 237312, 237313, 237314) c6( ) c7(bM, G, T, aC, fR, if, al, bB, ci, x, Vp, aE, il, Bc, pX, ajv, K, pk, dF, eL, aiJ, cp, de, eG, aW, az, g, eF, eM, nB, AM, iT, vA, U, cj, k, m, wu, zX, aw, yG, ht, n, aj, fr, qN, JW, eW, ft, no, jz, gd, h, lG, eV, hn, dl, ox, on, pl, afu, wC, Lh, aq, Hu, zZ, op, iO, bP, anC, hL, hR, E, at, it, B, fM, eX, aA, cz, hy, bX, V, q, J, eA, HC, aT, cy, rO, jj, bq, an, ae, af, ne, kl, s, iU, Y, r, en, cl, t, ik, ck, sS, iQ, hc, anD, fD, im, bt, LS, id, qb, iW, jh, dG, Fb, ad, av, hd, hD, wm, uD, d, BN, BO, ff, eH, KD, aF, iV, iR, bz, bF, bE, gZ, bC, y, X); #c1(BAZ1A) c2(NP_038476) c3(bromodomain adjacent to zinc finger domain protein IA isoform a) c4(176667) c5(176668, 176669, 176670, 176671, 176672, 176673, 176674, 176675, 176676, 176677, 176678) c6( ) c7(fr); #c1(BAZ1A) c2(NP_872589) c3(bromodomain adjacent to zinc finger domain protein IA isoform b) c4(256443, 256444) c5(256445, 256446, 256447, 256448, 256449, 256450, 256451, 256452, 256453, 256454, 256455, 256456) c6( ) c7(fr); #c1(BAZ1B) c2(NP_115784) c3(tyrosine-protein kinase BAZ1B) c4(223881) c5(223882, 223883, 223884, 223885, 223886, 223887, 223888, 223889, 223890, 223891, 223892, 223893, 223894, 223895) c6( ) c7(bL, alC, cO, e, dr, bN); #c1(BAZ2A) c2(NP_001287834) c3(bromodomain adjacent to zinc finger domain protein ZA isoform 2) c4(112986) c5(112987, 112988, 112989, 112990, 112991, 112992) c6( ) c7(ad); #c1(BAZ2A) c2(NP_038477) c3(bromodomain adjacent to zinc finger domain protein 2A isoform 1) c4(176679) c5(176680, 176681, 176682, 176683, 176684, 176685, 176686) c6( ) c7(ad); #c1(BAZ2A) c2(XP_011536101) c3(bromodomain adjacent to zinc finger domain protein 2A isoform X3) c4(333426) c5(333427, 333428, 333429, 333430, 333431, 333432) c6( ) c7(ad); #c1(BAZ2B) c2(NP_001276904) c3(bromodomain adjacent to zinc finger domain protein 28 isoform b) c4(108590, 108591) c5(108592, 108593, 108594, 108595, 108596, 108597, 108598, 108599, 108600, 108601, 108602, 108603) c6( ) c7(fq); #c1(BAZ2B) c2(NP_038478) c3(bromodomain adjacent to zinc finger domain protein 28 isoform a) c4(176687) c5(176688, 176689, 176690, 176691, 176692, 176693, 176694) c6( ) c7(fq); #c1(BAZ2B) c2(XP_011509339) c3(bromodomain adjacent to zinc finger domain protein 28 isoform X1) c4( ) c5(299918) c6( ) c7(fq); #c1(BAZ2B) c2(XP_011509350) c3(bromodomain adjacent to zinc finger domain protein 28 isoform X12) c4(299919) c5(299920, 299921, 299922, 299923, 299924, 299925) c6( ) c7(fq); #c1(BAZ2B) c2(XP_011509351) c3(bromodomain adjacent to zinc finger domain protein 2B isoform X13) c4(299926) c5( ) c6( ) c7(fq); #c1(BAZ2B) c2(XP_011509352) c3(bromodomain adjacent to zinc finger domain protein 28 isoform X14) c4(299927) c5(299928, 299929, 299930, 299931, 299932, 299933) c6( ) c7(fq); #c1(BAZ2B) c2(XP_011509357) c3(bromodomain adjacent to zinc finger domain protein 28 isoform X19) c4( ) c5(299934, 299935, 299936, 299937, 299938) c6( ) c7(fq); #c1(BBC3) c2(NP_001120712) c3(bcl-2-binding component 3 isoform 1) c4(44702) c5(44703) c6( ) c7(T, fM, fR, if, ht, jh, aA, bB, n, hy, fr, ci, C, qN, q, ad, eA, aE, aT, io, K, hB, ZN, nJ, ae, pk, eu, d, aW, Y, ff, r, GA, aq, vs, kp, aF, en, g, cm, iK, bz, KM, y, Kv, bE, cj, hE, bt, B, Ex); #c1(BBC3) c2(NP_001120713) c3(bcl-2-binding component 3 isoform 2) c4(44704) c5(44705, 44706, 44707, 44708, 44709, 44710) c6( ) c7(T, fM, FR, if, ht, jh, aA, bB, n, hy, fr, ci, C, qN, q, ad, eA, aE, aT, io, K, hB, ZN, nJ, ae, pk, eu, d, aW, Y, ff, r, GA, aq, vs, kp, aF, en, g, cm, ik, bz, KM, y, Kv, bE, cj, hE, bt, B, Ex); #c1(BBC3) c2(NP_001120714) c3(bcl-2-binding component 3 isoform 3) c4(44711) c5(44712, 44713, 44714, 44715, 44716, 44717, 44718, 44719, 44720, 44721, 44722) c6( ) c7(T, fM, fR, if, ht, jh, aA, bB, n, hy, fr, ci, C, qN, q, ad, eA, aE, aT, io, K, hB, ZN, nJ, ae, pk, eu, d, aW, Y, ff, r, GA, aq, vs, kp, aF, en, g, cm, ik, bz, KM, y, Kv, bE, cj, hE, bt, B, Ex); #c1(BBC3) c2(XP_006723204) c3(bcl-2-binding component 3 isoform X1) c4( ) c5(295036, 295037, 295038, 295039, 295040, 295041) c6( ) c7(T, fM, fR, if, ht, jh, aA, bB, n, hy, fr, ci, C, qN, q, ad, eA, aE, aT, io, K, hB, ZN, nJ, ae, pk, eu, d, aW, Y, ff, r, GA, aq, vs, kp, aF, en, g, cm, iK, bz, KM, y, Kv, bE, cj, hE, bt, B, Ex); #c1(BBC3) c2(XP_011525024) c3(bcl-2-binding component 3 isoform X2) c4(319485) c5(319486, 319487) c6( ) c7(T, fM, fR, if, ht, jh, aA, bB, n, hy, fr, ci, C, qN, q, ad, eA, aE, aT, io, K, hB, ZN, nJ, ae, pk, eu, d, aW, Y, ff, r, GA, aq, vs, kp, aF, en, g, cm, ik, bz, KM, y, Kv, bE, cj, hE, bt, B, Ex); #c1(BBC3) c2(XP_011525025) c3(bcl-2-binding component 3 isoform X3) c4( ) c5(319488) c6( ) c7(T, fM, fR, if, ht, jh, aA, bB, n, hy, fr, ci, C, qN, q, ad, eA, aE, aT, io, K, hB, ZN, nJ, ae, pk, eu, d, aW, Y, ff, r, GA, aq, vs, kp, aF, en, g, cm, iK, bz, KM, y, Kv, bE, cj, hE, bt, B, Ex); #c1(BBIP1) c2(NP_001182233) c3(BBSome-interacting protein 1 isoform 1) c4(74732) c5(74733, 74734, 74735, 74736, 74737, 74738) c6( ) c7(Tp); #c1(BBIP1) c2(NP_001182235) c3(BBSome-interacting protein 1 isoform 2) c4(74739, 74740) c5(74741, 74742, 74743, 74744, 74745, 74746, 74747, 74748) c6( ) c7(Tp); #c1(BBIP1) c2(NP_001182236) c3(BBSome-interacting protein 1 isoform 3) c4(74749) c5(74750, 74751, 74752, 74753, 74754, 74755) c6( ) c7(Tp); #c1(BBIP1) c2(NP_001230712) c3(BBSome-interacting protein 1 isoform 4) c4( ) c5(86683, 86684, 86685, 86686, 86687, 86688, 86689, 86690) c6( ) c7(Tp); #c1(BBOX1) c2(XP_011518705) c3(gamma-butyrobetaine dioxygenase isoform X1) c4(313079, 313080) c5(313081, 313082, 313083, 313084, 313085, 313086, 313087, 313088, 313089, 313090, 313091, 313092, 313093, 313094, 313095, 313096, 313097, 313098, 313099, 313100, 313101, 313102, 313103, 313104) c6( ) c7(ac); #c1(BBS1) c2(NP_078925) c3(Bardet-Biedl syndrome 1 protein) c4(216134, 216135, 216136) c5(216137, 216138, 216139, 216140, 216141, 216142, 216143, 216144, 216145, 216146, 216147, 216148, 216149, 216150, 216151, 216152, 216153, 216154) c6( ) c7(nk, BC, IR, rT, anE, nw, v, anF, Tp, Uw, nC, UF); #c1(BBS10) c2(NP_078961) c3(Bardet-Biedl syndrome 10 protein) c4(216285) c5(216286, 216287, 216288, 216289, 216290, 216291) c6( ) c7(bL, lR, anG, EP, v, zG, Tp); #c1(BBS(2) c2(NP_689831) c3(Bardet-Biedl syndrome 12 protein) c4(244082, 244083) c5(244084, 244085, 244086, 244087, 244088, 244089, 244090, 244091, 244092, 244093, 244094, 244095, 244096, 244097, 244098, 244099) c6( ) c7(dy, Tp, anH); #c1(BBS2) c2(XP_005256137) c3(Bardet-Biedl syndrome 2 protein isoform X1) c4(273997) c5(273998, 273999, 274000, 274001, 274002, 274003) c6( ) c7(qQ, v, fr, nA, ad, anl, qM, Nj, ae, lR, ev, Tp, nk, cl, cm, nC, es, iA, bE, anJ, hl, ank, zG); #c1(BBS4) c2(NP_149017) c3(Bardet-Biedl syndrome 4 protein isoform 1) c4(226747, 226748, 226749) c5(226750, 226751, 226752, 226753, 226754, 226755, 226756, 226757, 226758, 226759, 226760, 226761, 226762, 226763, 226764, 226765, 226766, 226767, 226768) c6( ) c7(IR, e, qQ, aT, anl, nw, v, qM, Tp, nC, de); #c1(BBS4) c2(XP_011520152) c3(Bardet-Biedl syndrome 4 protein isoform X1) c4(315023) c5(315024, 315025, 315026, 315027, 315028) c6( ) c7(IR, e, qQ, aT, anl, nw, v, qM, Tp, nC, de); #c1(BBS5) c2(NP_689597) c3(Bardet-Biedl syndrome 5 protein) c4(243161, 243162, 243163) c5(243164, 243165, 243166, 243167, 243168, 243169, 243/70, 243171, 243172, 243173, 243174, 243175, 243176, 243177, 243178, 243179, 243180, 243181) c6( ) c7(v, lR, Tp); #c1(BBS7) c2(NP_060660) c3(Bardet-Biedl syndrome 7 protein isoform b) c4( ) c5(197029, 197030, 197031, 197032, 197033, 197034) c6( ) c7(anM, lR, v, Tp, nA, anN); #c1(BBS7) c2(NP_789794) c3(Bardet-Biedl syndrome 7 protein isoform a) c4(252334, 252335) c5(252336, 252337, 252338, 252339, 252340, 252341) c6( ) c7(anM, lR, v, Tp, nA, anN); #c1(BBS7) c2(XP_011530383) c3(Bardet-Biedl syndrome 7 protein isoform X4) c4(326220) c5(326221, 326222, 326223, 326224, 326225, 326226) c6( ) c7(anM, lR, v, Tp, nA, anN); #c1(BBS9) c2(NP_001028776) c3(protein PTHB1 isoform 3) c4(30657, 30658) c5(30659, 30660, 30661, 30662, 30663, 30664, 30665, 30666, 30667, 30668, 30669, 30670) c6( ) c7(ul, lR, pN, il, oe, and, v, zV, Tp, zZ, nC, er); #c1(BBS9) c2(NP_001028777) c3(protein PTHB1 isoform 4) c4(30671, 30672) c5(30673, 30674, 30675, 30676, 30677, 30678) c6( ) c7(ul, lR, pN, il, oe, and, v, zV, Tp, zZ, nC, er); #c1(BBS9) c2(NP_055266) c3(protein PTHB1 isoform 1) c4(180154, 180155) c5(180156, 180157, 180158, 180159, 180160, 180161, 180162, 180163, 180164, 180165, 180166, 180167) c6( ) c7(ul, lR, pN, il, oe, and, v, zV, Tp, zZ, nC, er); #c1(BBS9) c2(NP_940820) c3 (protein PTHB1 isoform 2) c4(259920, 259921) c5(259922, 259923, 259924, 259925, 259926, 259927, 259928, 259929, 259930, 259931, 259932, 259933) c6( ) c7(ul, lR, pN, il, oe, and, v, zV, Tp, zZ, nC, er); #c1(BBS9) c2(XP_005249757) c3(protein PTHB1 isoform X3) c4(269797, 269798) c5( ) c6( ) c7(ul, lR, pN, il, oe, and, v, zV, Tp, zZ, nC, er); #c1(BBS9) c2(XP_005249758) c3(protein PTHB1 isoform X8) c4( ) c5(269799, 269800, 269801, 269802, 269803, 269804, 269805, 269806, 269807, 269808, 269809, 269810, 269811, 269812, 269813, 269814, 269815) c6( ) c7(ul, lR, pN, il, oe, and, v, zV, Tp, zZ, nC, er); #c1(BBS9) c2(XP_011513567) c3(protein PTHB1 isoform X1) c4( ) c5(305876) c6( ) c7(ul, lR, pN, il, oe, and, v, zV, Tp, zZ, nC, er); #c1(BBS9) c2(XP_011513571) c3(protein PTHB1 isoform X6) c4(305877) c5(305878, 305879, 305880, 305881, 305882, 305883) c6( ) c7(ul, lR, pN, il, oe, and, v, zV, Tp, zZ, nC, er); #c1(BBX) c2(NP_001136040) c3(HMG box transcription factor BBX isoform 1) c4(54133) c5(54134, 54135, 54136, 54137, 54138, 54139) c6( ) c7(bw, Y, fq, Ea); #c1(BBX) c2(NP_001263215) c3(HMG box transcription factor BBX isoform 3) c4(97042) c5( ) c6( ) c7(bw, Y, fq, Ea); #c1(BBX) c2(NP_064620) c3(HMG box transcription factor BBX isoform 2) c4(203320, 203321) c5(203322, 203323, 203324, 203325, 203326, 203327, 203328, 203329, 203330, 203331) c6( ) c7(bw, Y, fq, Ea); #c1(BBX) c2(XP_011511300) c3(HMG box transcription factor BBX isoform X3) c4(301823) c5(301824, 301825, 301826, 301827, 301828, 301829, 301830, 301831, 301832, 301833, 301834, 301835, 301836, 301837, 301838, 301839, 301840, 301841, 301842) c6( ) c7(bw, Y, fq, Ea); #c1(BBX) c2(XP_011511301) c3(HMG box transcription factor BBX isoform X4) c4(301843, 301844, 301845, 301846) c5( ) c6( ) c7(bw, Y, fq, Ea); #c1(BBX) c2(XP_011511304) c3(HMG box transcription factor BBX isoform X5) c4(301847, 301848) c5( ) c6( ) c7(bw, Y, fq, Ea); #c1(BBX) c2(XP_011511307) c3(HMG box transcription factor BBX isoform X7) c4(301849, 301850, 301851, 301852, 301853) c5(301854, 301855, 301856, 301857, 301858, 301859, 301860, 301861, 301862, 301863) c6( ) c7(bw, Y, fq, Ea); #c1(BBX) c2(XP_011511311) c3(HMG box transcription factor BBX isoform X11) c4( ) c5(301864) c6( ) c7(bw, Y, fq, Ea); #c1(BBX) c2(XP_011511312) c3(HMG box transcription factor BBX isoform X12) c4(301865) c5(301866, 301867, 301868, 301869, 301870, 301871, 301872, 301873, 301874, 301875, 301876, 301877, 301878, 301879, 301880, 301881, 301882, 301883, 301884, 301885, 301886, 301887, 301888, 301889, 301890, 301891, 301892, 301893, 301894, 301895, 301896, 301897, 301898, 301899, 301900, 301901, 301902, 301903, 301904, 301905, 301906, 301907, 301908, 301909, 301910, 301911, 301912, 301913, 301914, 301915, 301916, 301917, 301918, 301919, 301920, 301921, 301922, 301923, 301924, 301925, 301926) c6( ) c7(bw, Y, fq, Ea); #c1(BCAM) c2(NP_001013275) c3(basal cell adhesion molecule isoform 2 precursor) c4(25501) c5(25502, 25503, 25504, 25505, 25506, 25507) c6( ) c7(T, anP, qb, bE, e, bB, aq, and, pm, fq, B); #c1(BCAM) c2(NP_005572) c3(basal cell adhesion molecule isoform 1 precursor) c4(157571) c5(157572, 157573, 157574, 157575, 157576, 157577) c6( ) c7(T, anP, qb, bE, e, bB, aq, and, pm, fq, B); #c1(BCAN) c2(NP_940819) c3(brevican core protein isoform 2 precursor) c4(259865, 259866, 259867, 259868, 259869) c5(259870, 259871, 259872, 259873, 259874, 259875, 259876, 259877, 259878, 259879, 259880, 259881, 259882, 259883, 259884, 259885, 259886, 259887, 259888, 259889, 259890, 259891, 259892, 259893, 259894, 259895, 259896, 259897, 259898, 259899, 259900, 259901, 259902, 259903, 259904, 259905, 259906, 259907, 259908, 259909) c6(259910, 259911, 259912, 259913, 259914, 259915, 259916, 259917, 259918, 259919) c7(d, ac, dB, dL, Fc, eL, eA, eu); #c1(BCAN) c2(XP_011508168) c3(brevican core protein isoform X1) c4(298708) c5(298709, 298710, 298711, 298712, 298713, 298714, 298715, 298716, 298717, 298718) c6(298719, 298720) c7(d, ac, dB, dL, Fc, eL, eA, eu); #c1(BCAP29) c2(NP_001008405) c3(B-cell receptor-associated protein 29 isoform a) c4(23165) c5(23166, 23167, 23168, 23169, 23170, 23171, 23172, 23173, 23174, 23175, 23176, 23177, 23178) c6( ) c7(cF, j); #c1(BCAP29) c2(NP_061332) c3(B-cell receptor-associated protein 29 isoform b) c4(200059, 200060) c5(200061, 200062, 200063, 200064, 200065, 200066, 200067, 200068, 200069, 200070, 200071, 200072, 200073, 200074, 200075, 200076, 200077, 200078, 200079, 200080, 200081, 200082, 200083, 200084, 200085, 200086, 200087, 200088, 200089, 200090, 200091, 200092, 200093, 200094, 200095, 200096, 200097, 200098, 200099) c6(200100, 200101) c7(cF, j); #c1(BCAP29) c2(XP_011514709) c3(B-cell receptor-associated protein 29 isoform X2) c4(307348, 307349, 307350, 307351) c5( ) c6( ) c7(cF, j); #c1(BCAP29) c2(XP_011514711) c3(B-cell receptor-associated protein 29 isoform X4) c4(307352, 307353) c5(307354, 307355, 307356, 307357, 307358, 307359, 307360, 307361, 307362, 307363, 307364) c6( ) c7(cF, j); #c1(BCAP31) c2(NP_001132929) c3(B-cell receptor-associated protein 31 isoform a) c4(52651, 52652, 52653, 52654, 52655, 52656) c5(52657, 52658, 52659, 52660, 52661, 52662, 52663, 52664, 52665, 52666, 52667, 52668, 52669, 52670, 52671, 52672, 52673) c6( ) c7(T, anR, anS, anT, fM, iM, aW, je, mG, oj, aT, aU, Be, hu, dF, B, hX); #c1(BCAP31) c2(NP_001243376) c3(B-cell receptor-associated protein 31 isoform b) c4(90592) c5(90593, 90594, 90595, 90596, 90597, 90598, 90599, 90600, 90601, 90602, 90603, 90604, 90605, 90606, 90607, 90608, 90609, 90610, 90611, 90612, 90613, 90614, 90615, 90616, 90617, 90618, 90619, 90620, 90621, 90622, 90623, 90624, 90625, 90626, 90627, 90628, 90629, 90630, 90631) c6( ) c7(T, anR, anS, anT, fM, iM, aW, je, mG, oj, aT, aU, Be, hu, dF, B, hX); #c1(BCAR1) c2(NP_001164185) c3(breast cancer anti-estrogen resistance protein 1 isoform 1) c4(66720) c5(66721, 66722, 66723, 66724, 66725, 66726) c6( ) c7(bL, T, aC, sw, jh, bB, n, j, ci, fr, AD, q, ad, eA, anU, yt, anV, aT, h, dL, ae, dx, d, aiB, s, anX, aW, Y, vj, qt, ff, r, ks, BG, en, anW, g, t, HI, cm, ck, kP, eM, bE, anY, X, iS, np, m, y, B); #c1(BCAR1) c2(NP_001164186) c3(breast cancer anti-estrogen resistance protein 1 isoform 2) c4(66727) c5(66728, 66729, 66730, 66731, 66732, 66733) c6( ) c7(bL, T, aC, sw, jh, bB, n, j, ci, fr, AD, q, ad, eA, anU, yt, anV, aT, h, dL, ae, dx, d, aiB, s, anX, aW, Y, vj, qt, ff, r, ks, BG, en, anW, g, t, HI, cm, ck, kP, eM, bE, anY, X, iS, np, m, y, B); #c1(BCAR1) c2(NP_001164187) c3(breast cancer anti-estrogen resistance protein 1 isoform 3) c4(66734) c5(66735, 66736, 66737, 66738, 66739, 66740) c6( ) c7(bL, T, aC, sw, jh, bB, n, j, ci, fr, AO, q, ad, eA, anU, yt, anV, aT, h, dL, ae, dx, d, aiB, s, anX, aW, Y, vj, qt, ff, r, ks, BG, en, anW, g, t, HI, cm, ck, kP, eM, bE, anY, X, iS, np, m, y, B); #c1(BCAR1) c2(NP_001164188) c3(breast cancer anti-estrogen resistance protein 1 isoform 4) c4(66741) c5(66742, 66743, 66744, 66745, 66746, 66747) c6( ) c7(bL, T, aC, sw, jh, bB, n, j, ci, fr, AO, q, ad, eA, anU, yt, anV, aT, h, dL, ae, dx, d, aiB, s, anX, aW, Y, vj, qt, ff, r, ks, BG, en, anW, g, t, HI, cm, ck, kP, eM, bE, anY, X, iS, np, m, y, B); #c1(BCAR1) c2(NP_001164189) c3(breast cancer anti-estrogen resistance protein 1 isoform 5) c4(66748) c5(66749, 66750, 66751, 66752, 66753, 66754) c6( ) c7(bL, T, aC, sw, jh, bB, n, j, ci, fr, AO, q, ad, eA, anU, yt, anV, aT, h, dL, ae, dx, d, aiB, s, anX, aW, Y, vj, qt, ff, r, ks, BG, en, anW, g, t, HI, cm, ck, kP, eM, bE, anY, X, iS, np, m, y, B); #c1(BCAR1) c2(NP_001164190) c3(breast cancer anti-estrogen resistance protein 1 isoform 7) c4(66755) c5(66756, 66757, 66758, 66759, 66760, 66761) c6( ) c7(bL, T, aC, sw, jh, bB, n, j, ci, fr, AO, q, ad, eA, anU, yt, anV, aT, h, dL, ae, dx, d, aiB, s, anX, aW, Y, vj, qt, ff, r, ks, BG, en, anW, g, t, HI, cm, ck, kP, eM, bE, anY, X, iS, np, m, y, B); #c1(BCAR1) c2(NP_001164191) c3(breast cancer anti-estrogen resistance protein 1 isoform 8) c4(66762) c5(66763, 66764, 66765, 66766, 66767, 66768) c6( ) c7(bL, T, aC, sw, jh, bB, n, j, ci, fr, AD, q, ad, eA, anU, yt, anV, aT, h, dL, ae, dx, d, aiB, s, anX, aW, Y, vj, qt, ff, r, ks, BG, en, anW, g, t, HI, cm, ck, kP, eM, bE, anY, X, iS, np, m, y, B); #c1(BCAR1) c2(NP_055382) c3(breast cancer anti-estrogen resistance protein 1 isoform 6) c4(180694) c5(180695, 180696, 180697, 180698, 180699, 180700, 180701, 180702) c6(180703, 180704) c7(bL, T, aC, sw, jh, bB, n, j, ci, fr, AD, q, ad, eA, anU, yt, anV, aT, h, dL, ae, dx, d, aiB, s, anX, aW, Y, vj, qt, ff, r, ks, BG, en, anW, g, t, HI, cm, ck, kP, eM, bE, anY, X, iS, np, m, y, B); #c1(BCAR1) c2(XP_005256315) c3(breast cancer anti-estrogen resistance protein 1 isoform X2) c4( ) c5(274094) c6( ) c7(bL, T, aC, sw, jh, bB, n, j, ci, fr, AO, q, ad, eA, anU, yt, anV, aT, h, dL, ae, dx, d, aiB, s, anX, aW, Y, vj, qt, ff, r, ks, BG, en, anW, g, t, HI, cm, ck, kP, eM, bE, anY, X, iS, np, m, y, B); #c1(BCAR1) c2(XP_005256316) c3(breast cancer anti-estrogen resistance protein 1 isoform X3) c4(274095) c5( ) c6( ) c7(bL, T, aC, sw, jh, bB, n, j, ci, fr, AO, q, ad, eA, anU, yt, anV, aT, h, dL, ae, dx, d, aiB, s, anX, aW, Y, vj, qt, ff, r, ks, BG, en, anW, g, t, HI, cm, ck, kP, eM, bE, anY, X, iS, np, m, y, B); #c1 (BCAR1) c2(XP_011521777) c3(breast cancer anti-estrogen resistance protein 1 isoform X5) c4( ) c5(316468, 316469, 316470, 316471, 316472) c6( ) c7(bL, T, aC, sw, jh, bB, n, j, ci, fr, AO, q, ad, eA, anU, yt, anV, aT, h, dL, ae, dx, d, aiB, s, anX, aW, Y, vj, qt, ff, r, ks, BG, en, anW, g, t, HI, cm, ck, kP, eM, bE, anY, X, iS, np, m, y, B); #c1(BCAS1) c2(NP_003648) c3(breast carcinoma-amplified sequence 1) c4(139916, 139917) c5(139918, 139919, 139920, 139921, 139922, 139923) c6( ) c7(T, c, aW, aw, aT, j, ad); #c1(BCAS1) c2(XP_005260648) c3(breast carcinoma-amplified sequence 1 isoform X4) c4(276772) c5(276773, 276774, 276775, 276776, 276777, 276778) c6( ) c7(T, c, aW, aw, aT, j, ad); #c1(BCAS1) c2(XP_005260652) c3(breast carcinoma-amplified sequence 1 isoform X8) c4(276779) c5(276780, 276781, 276782, 276783, 276784, 276785) c6( ) c7(T, c, aW, aw, aT, j, ad); #c1(BCAS3) c2(NP_001092902) c3(breast carcinoma-amplified sequence 3 isoform 1) c4(39742, 39743) c5(39744, 39745, 39746, 39747, 39748, 39749) c6( ) c7(T, Mo, ac, eR, Cc, aW, anZ, dB, aT, aj); #c1(BCAS3) c2(NP_060149) c3(breast carcinoma-amplified sequence 3 isoform 2) c4(194496) c5(194497, 194498, 194499, 194500, 194501, 194502, 194503) c6( ) c7(T, Mo, ac, eR, Cc, aW, anZ, dB, aT, aj); #c1(BCAS3) c2(XP_005257529) c3(breast carcinoma-amplified sequence 3 isoform X7) c4( ) c5(275213, 275214, 275215, 275216, 275217, 275218, 275219, 275220, 275221, 275222, 275223, 275224, 275225, 275226, 275227, 275228, 275229, 275230) c6( ) c7(T, Mo, ac, eR, Cc, aW, anZ, dB, aT, aj); #c1(BCAS3) c2(XP_005257532) c3(breast carcinoma-amplified sequence 3 isoform X9) c4(275231) c5( ) c6( ) c7(T, Mo, ac, eR, Cc, aW, anZ, dB, aT, aj); #c1 (BCAS3) c2(XP_011523242) c3(breast carcinoma-amplified sequence 3 isoform X2) c4(317764) c5( ) c6( ) c7(T, Mo, ac, eR, Cc, aW, anZ, dB, aT, aj); #c1(BCAS3) c2(XP_011523247) c3(breast carcinoma-amplified sequence 3 isoform X8) c4(317765) c5( ) c6( ) c7(T, Mo, ac, eR, Cc, aW, anZ, dB, aT, aj); #c1(BCAS3) c2(XP_011523253) c3(breast carcinoma-amplified sequence 3 isoform X15) c4( ) c5(317766, 317767, 317768, 317769, 317770) c6( ) c7(T, Mo, ac, eR, Cc, aW, anZ, dB, aT, aj); #c1(BCAS3) c2(XP_011523254) c3(breast carcinoma-amplified sequence 3 isoform X16) c4(317771) c5(317772, 317773, 317774, 317775, 317776, 317777) c6( ) c7(T, Mo, ac, eR, Cc, aW, anZ, dB, aT, aj); #c1(BCAS4) c2(NP_001010974) c3(breast carcinoma-amplified sequence 4 isoform c) c4(24381) c5(24382, 24383, 24384, 24385, 24386, 24387) c6( ) c7(aW, j, nA, aT); #c1(BCAS4) c2(NP_060313) c3(breast carcinoma-amplified sequence 4 isoform a) c4(195358, 195359) c5(195360, 195361, 195362, 195363, 195364, 195365, 195366, 195367, 195368, 195369, 195370, 195371) c6( ) c7(aW, j, nA, aT); #c1(BCAS4) c2(XP_011527189) c3(breast carcinoma-amplified sequence 4 isoform X2) c4(321719) c5(321720, 321721, 321722, 321723, 321724, 321725) c6( ) c7(aW, j, nA, aT); #c1(BCAT1) c2(NP_001171562) c3(branched-chain-amino-acid aminotransferase, cytosolic isoform 2) c4(70588) c5(70589, 70590, 70591, 70592, 70593, 70594, 70595) c6( ) c7(jf, A, d, ac, ey, ci, eA); #c1(BCAT1) c2(NP_001171563) c3(branched-chain-amino-acid aminotransferase, cytosolic isoform 3) c4(70596) c5(70597, 70598, 70599, 70600, 70601) c6( ) c7(jf, A, d, ac, ey, ci, eA); #c1(BCAT1) c2(NP_001171565) c3(branched-chain-amino-acid aminotransferase, cytosolic isoform 5) c4(70602) c5(70603, 70604, 70605, 70606, 70607, 70608) c6( ) c7(jf, A, d, ac, ey, ci, eA); #c1(BCAT1) c2(NP_005495) c3(branched-chain-amino-acid aminotransferase, cytosolic isoform 1) c4(156787) c5(156788, 156789, 156790, 156791, 156792, 156793) c6( ) c7(jf, A, d, ac, ey, ci, eA); #c1(BCAT1) c2(XP_011519112) c3(branched-chain-amino-acid aminotransferase, cytosolic isoform X1) c4(313717) c5(313718, 313719, 313720, 313721, 313722, 313723) c6( ) c7(jf, A, d, ac, ey, ci, eA); #c1(BCAT2) c2(NP_001158245) c3(branched-chain-amino-acid aminotransferase, mitochondrial isoform b) c4(63508) c5(63509, 63510, 63511, 63512, 63513, 63514) c6( ) c7(qb, v, agm, pm); #c1(BCAT2) c2(NP_001271254) c3(branched-chain-amino-acid aminotransferase, mitochondrial isoform c) c4( ) c5(103753, 103754, 103755, 103756, 103757) c6( ) c7(qb, v, agm, pm); #c1(BCCIP) c2(NP_057651) c3(BRCA2 and CDKN1A-interacting protein isoform BCCIPalpha) c4(192056) c5(192057, 192058, 192059, 192060, 192061, 192062) c6( ) c7(T, d, NE, dB, aW, Y, go, hk, afQ, afd, aT, r, ff, j, K, m, en, as, t, q, ad); #c1 (BCCIP) c2(NP_510868) c3(BRCA2 and CDKN1A-interacting protein isoform BCCIPbeta) c4(231039, 231040) c5(231041, 231042, 231043, 231044, 231045, 231046, 231047, 231048, 231049, 231050, 231051, 231052) c6( ) c7(T, d, NE, dB, aW, Y, go, hk, afl, afd, aT, r, ff, j, K, m, en, as, t, q, ad); #c1(BCCIP) c2(NP_510869) c3(BRCA2 and CDKN1A-interacting protein isoform C) c4(231053, 231054) c5(231055, 231056, 231057, 231058, 231059, 231060, 231061, 231062, 231063, 231064, 231065, 231066) c6( ) c7(T, d, NE, dB, aW, Y, go, hk, afQ, afd, aT, r, ff, j, K, m, en, as, t, q, ad); #c1(BCDIN3D) c2(NP_859059) c3(pre-miRNA 5′-monophosphate methyltransferase) c4(255441) c5(255442, 255443, 255444, 255445, 255446, 255447) c6( ) c7(bL, bM, fq, v); #c1(BCHE) c2(NP_000046) c3(cholinesterase precursor) c4(743) c5(744, 745, 746, 747, 748, 749, 750, 751, 752, 753, 754, 755, 756, 757, 758, 759, 760, 761, 762, 763, 764, 765) c6(766) c7(T, fV, al, l, aoj, ci, W, xy, wM, dv, K, H, CG, aoa, ZH, aW, lW, hl, g, bw, cm, yX, wE, U, cj, m, akA, aj, fr, aoc, aoh, on, Jj, CN, wZ, bJ, KT, ok, xA, Qu, kz, FV, fL, L, bL, eR, jx, v, j, ane, V, q, J, ga, aod, aT, bq, af, ak, hM, qw, cu, t, aoi, ck, ac, bt, ao, aof, gx, sO, abq, rN, cx, d, aog, xs, eZ, kD, aob, iq, es, bE, dJ, ei, xg); #c1(BCHE) c2(XP_005247742) c3(cholinesterase isoform X1) c4(267953, 267954) c5(267955) c6( ) c7(T, fV, al, l, aoj, ci, VV, xy, wM, dv, K, H, CG, aoa, ZH, aW, lW, hl, g, bw, cm, yX, wE, U, cj, m, akA, aj, fr, aoc, aoh, on, Jj, CN, wZ, bJ, KT, ok, xA, Qu, kz, FV, FL, L, bL, eR, jx, v, j, ane, V, q, J, ga, aod, aT, bq, af, ak, hM, qw, cu, t, aoi, ck, ac, bt, ao, aof, gx, sO, abq, rN, cx, d, aog, xs, eZ, kD, aob, iq, es, bE, dJ, ei, xg); #c1(BCKDHA) c2(NP_000700) c3(2-oxoisovalerate dehydrogenase subunit alpha, mitochondrial isoform 1 precursor) c4(14627, 14628, 14629, 14630) c5(14631, 14632, 14633, 14634, 14635, 14636, 14637, 14638, 14639, 14640, 14641, 14642, 14643, 14644, 14645, 14646, 14647, 14648, 14649, 14650, 14651, 14652, 14653, 14654, 14655, 14656, 14657, 14658, 14659, 14660, 14661, 14662, 14663, 14664, 14665, 14666, 14667, 14668, 14669, 14670, 14671, 14672, 14673, 14674, 14675, 14676, 14677, 14678, 14679, 14680, 14681, 14682, 14683, 14684, 14685, 14686, 14687, 14688, 14689, 14690, 14691, 14692, 14693, 14694, 14695, 14696, 14697, 14698, 14699, 14700, 14701, 14702, 14703, 14704, 14705, 14706, 14707, 14708, 14709, 14710, 14711, 14712, 14713, 14714, 14715, 14716, 14717, 14718, 14719, 14720, 14721, 14722, 14723, 14724, 14725, 14726, 14727, 14728, 14729) c6(14730) c7(aok, CT, agm, bq); #c1(BCKDHB) c2(NP_000047) c3(2-oxoisovalerate dehydrogenase subunit beta, mitochondrial precursor) c4(767, 768) c5(769, 770, 771, 772, 773, 774, 775, 776, 777, 778, 779, 780) c6( ) c7(T, aE, lg, il, aW, eZ, aT, agm, bq, hX); #c1(BCKDHB) c2(XP_011534328) c3(2-oxoisovalerate dehydrogenase subunit beta, mitochondrial isoform X5) c4( ) c5(330698, 330699, 330700, 330701, 330702) c6( ) c7(T, aE, lg, il, aW, eZ, aT, agm, bq, hX); #c1(BCKDHB) c2(XP_011534329) c3(2-oxoisovalerate dehydrogenase subunit beta, mitochondrial isoform X6) c4(330703) c5(330704, 330705, 330706, 330707, 330708, 330709, 330710, 330711, 330712) c6( ) c7(T, aE, lg, il, aW, eZ, aT, agm, bq, hX); #c1(BCKDK) c2(NP_001116429) c3([3-methyl-2-oxobutanoate dehydrogenase [lipoamide] kinase, mitochondrial isoform b precursor) c4( ) c5(43877, 43878, 43879, 43880, 43881) c6( ) c7(bN, nA, ha, aol); #c1 (BCKDK) c2(NP_001258855) c3([3-methyl-2-oxobutanoate dehydrogenase [lipoamide] kinase, mitochondrial isoform c) c4(96590) c5(96591, 96592, 96593, 96594, 96595) c6( ) c7(bN, nA, ha, aol); #c1(BCKDK) c2(NP_005872) c3([3-methyl-2-oxobutanoate dehydrogenase [lipoamide]] kinase, mitochondrial isoform a precursor) c4(160046, 160047) c5(160048, 160049) 160050, 160051, 160052, 160053, 160054, 160055, 160056, 160057, 160058, 160059) c6( ) c7(bN, nA, ha, aol); #c1(BCL10) c2(XP_011540701) c3(B-cell lymphoma/leukemia 10 isoform X4) c4( ) c5(339270, 339271) c6(339272, 339273) c7(T, fM, aoq, aD, if, ht, jh, aA, bB, n, aj, fd, ci, qN, aon, V, q, J, ad, eA, x, AW, ft, aop, xy, ib, aE, pe, fm, ajv, aT, kf, h, iY, K, hu, ae, YT, iU, vT, aW, Y, aoo, az, oV, ff, r, aq, af, en, aom, bu, t, cl, KM, bz, g, Ld, iK, ck, es, eU, amS, c, bE, cj, aor, m, bt, it, B, hS); #c1(BCL11A) c2(NP_060484) c3(B-cell lymphoma/leukemia 11A isoform 2) c4(196202, 196203, 196204) c5(196205, 196206, 196207, 196208, 196209, 196210, 196211, 196212, 196213, 196214, 196215, 196216, 196217, 196218, 196219, 196220, 196221, 196222, 196223) c6( ) c7(bL, T, fM, qb, eX, jh, rf, x, qK, oW, h, eV, aos, bK, pk, pq, kl, aot, ck, BU, qe, eM, iT, cj, k, ER, pw, bt); #c1(BCL11A) c2(NP_075044) c3(B-cell lymphoma/leukemia 11A isoform 1) c4(213543) c5(213544, 213545, 213546, 213547, 213548, 213549) c6( ) c7(bL, T, fM, qb, eX, jh, rf, x, qK, oW, h, eV, aos, bK, pk, pq, kl, aot, ck, BU, qe, eM, iT, cj, k, ER, pw, bt); #c1(BCL11A) c2(NP_612569) c3(B-cell lymphoma/leukemia 11A isoform 3) c4(236704) c5(236705, 236706, 236707, 236708, 236709, 236710) c6( ) c7(bL, T, fM, qb, eX, jh, rf, x, qK, oW, h, eV, aos, bK, pk, pq, kl, aot, ck, BU, qe, eM, iT, cj, k, ER, pw, bt); #c1(BCL11A) c2(XP_011531213) c3(B-cell lymphoma/leukemia 1IA isoform X3) c4(327180) c5(327181, 327182, 327183, 327184, 327185, 327186) c6( ) c7(bL, T, fM, qb, ex, jh, rf, x, qK, oW, h, eV, aos, bK, pk, pq, kl, aot, ck, BU, qe, eM, iT, cj, k, ER, pw, bt); #c1 (BCL11A) c2(XP_011531214) c3(B-cell lymphoma/leukemia 11A isoform X4) c4(327187) c5(327188, 327189, 327190, 327191, 327192, 327193) c6( ) c7(bL, T, fM, qb, ex, jh, rf, x, qK, oW, h, eV, aos, bK, pk, pq, kl, aot, ck, BU, qe, eM, iT, cj, k, ER, pw, bt); #c1(BCL11B) c2(NP_075049) c3(B-cell lymphoma/leukemia 11B isoform 2) c4(213578) c5(213579, 213580, 213581, 213582, 213583, 213584) c6( ) c7(T, aiY, Pt, yS, e, jh, oY, ci, hF, aje, x, kH, h, dx, je, iU, Az, dN, fe, hf, eE, iK, ck, iM, cj, k, bD, bt); #c1(BCL11B) c2(NP_612808) c3(B-cell lymphoma/leukemia 11B isoform 1) c4(236769) c5(236770, 236771, 236772, 236773, 236774, 236775) c6( ) c7(T, aiY, Pt, yS, e, jh, oY, ci, hF, aje, x, kH, h, dx, je, iU, Az, dN, fe, hf, eE, ik, ck, iM, cj, k, bD, bt); #c1(BCL2) c2(NP_000624) c3(apoptosis regulator Bcl-2 alpha isoform) c4(14035) c5(14036, 14037, 14038, 14039, 14040, 14041, 14042, 14043, 14044, 14045, 14046, 14047, 14048, 14049) c6(14050, 14051, 14052, 14053) c7(gh, T, ea, bO, fd, Vd, ci, aY, YD, bv, xy, aE, ajv, aol, aok, dV, hu, hH, aiJ, cp, aa, li, ly, ZH, az, zC, zE, lk, iA, nB, cj, cX, k, fy, de, ht, cd, aj, PJ, br, aoc, aow, apG, Ak, lG, du, ey, Dz, dd, YT, pl, apo, MD, Lh, ajz, hq, iO, dB, aoB, apE, AN, jp, fZ, aA, ol, cz, bX, lu, AO, q, zn, akU, kf, cy, jj, rv, eu, gP, NE, nO, lo, eS, cl, Cv, rR, iM, ac, iQ, nG, aor, aps, bt, rA, ao, gV, iW, jN, jh, dG, Sv, Gn, ape, xt, ad, hX, aoH, afb, le, oW, wm, amY, ahK, aoZ, apw, iz, WZ, gj, cH, ff, Of, aph, dq, app, iV, bz, abA, lC, ne, jr, iu, Tc, oO, bM, lD, anl, ya, if, bB, aon, ip, x, Ma, ja, bc, Qe, YL, xQ, eG, Mq, JI, hl, kt, aoD, as, eE, eF, cm, bw, Qt, go, vA, apy, hh, Vj, dD, gU, wu, n, oY, aoQ, no, pe, jz, Bj, gd, eV, hn, gl, apD, cY, apc, oS, A, kj, fJ, GA, po, yq, YE, bP, apf, hL, at, SZ, fL, eo, cB, B, aoT, dA, eX, v, aoN, Qo, V, eJ, qH, xx, hW, BJ, api, ae, jf, Lu, vT, ev, bu, apx, t, ho, aoy, ck, kP, wO, au, gX, vi, Dd, OR, eq, Fc, aoz, BY, Tg, hd, CJ, py, db, LA, hD, nJ, d, je, hG, et, zR, aoF, aoW, dr, wW, bF, gk, wz, apv, rH, y, kS, hv, ng, aC, fR, hV, al, Qp, apF, Bc, pX, DO, K, aoV, apd, dF, eL, apl, aoo, apn, aB, aoY, apm, g, apB, aoS, iP, ou, AM, nF, U, dW, m, aov, QO, sw, OI, aw, NT, fN, qy, apt, apz, KL, ft, kH, cU, CN, aoX, ks, Og, YF, yR, apl, yP, it, aox, aoU, fM, aDE, jx, hz, j, il, pd, eA, er, XX, amg, lg, bq, aoP, kl, Y, r, aou, en, aoG, apg, iK, bN, QG, Dy, c, apu, hc, np, dy, fD, LS, gx, kh, id, aZ, bS, hF, iJ, vN, dP, fX, oj, io, apH, Yi, lR, xs, eH, cr, hp, iR, Nq, pu, hE, G, apr, pf, qx, aoM, XW, apj, oi, H, pk, Zm, hm, akV, aW, eM, iT, uJ, nw, zv, cZ, ajm, fr, kO, qN, ai, eW, ke, Bl, co, TK, h, dL, pj, hB, mY, Df, afu, Nr, aiB, iw, aq, apk, kp, Hu, mo, Ca, op, lx, aoO, apa, Qu, hk, anC, hR, ajW, YH, bL, yk, hy, C, ze, aoC, Zp, ga, mP, nv, Vr, aoA, HC, aT, vH, dz, bK, an, af, aol, ak, qw, s, iU, oV, hf, f, Ta, sS, apq, wg, nM, Zg, lV, aoJ, ij, hS, Kd, Ke, aD, ha, Me, vm, Fb, OH, apC, mC, KL, av, F, ot, MS, ib, sO, zK, NS, nl, Od, fb, iL, apA, TC, qt, eZ, apb, Ej, aF, ek, es, aoR, bE, dJ, uR, aS, XO, X, Ex); #c1(BCL2) c2(NP_000648) c3(apoptosis regulator Bcl-2 beta isoform) c4(14157) c5(14158, 14159, 14160, 14161, 14162, 14163, 14164, 14165, 14166, 14167) c6(14168, 14169) c7(gh, T, ea, bO, fd, Vd, ci, aY, YD, bv, xy, aE, ajv, aol, aok, dV, hu, hH, aiJ, cp, aa, li, ly, ZH, az, zC, zE, lk, iA, nB, cj, cX, k, fy, de, ht, cd, aj, PJ, br, aoc, aow, apG, Ak, lG, dU, ey, Dz, dd, YT, pl, apo, MD, Lh, ajz, hq, iO, dB, aoB, apE, AN, jp, fZ, aA, ol, cz, bX, lu, A, q, zn, akU, kf, cy, jj, rv, eu, gP, NE, nO, lo, eS, cl, Cv, rR, iM, ac, iQ, nG, aor, aps, bt, rA, ao, gV, iW, jN, jh, dG, Sv, Gn, ape, xt, ad, hX, aoH, afb, le, oW, wm, amY, ahK, aoZ, apw, iz, WZ, gj, cH, ff, Of, aph, dq, app, iV, bz, abA, lC, ne, jr, iu, Tc, oO, bM, lD, anl, ya, if, bB, aon, ip, x, Ma, ja, bc, Qe, YL, xQ, eG, Mq, JI, hl, kt, aoD, as, eE, eF, cm, bw, Qt, go, vA, apy, hh, Vj, dD, gU, wu, n, oY, aoQ, no, pe, jz, Bj, gd, eV, hn, gl, apD, cY, apc, oS, A, kj, fJ, GA, po, yq, YE, bP, apf, hL, at, SZ, fl, eo, cB, B, aoT, dA, eX, v, aoN, Qo, V, eJ, qH, xx, hW, BJ, api, ae, jf, Lu, vT, ev, bu, apx, t, ho, aoy, ck, kP, wD, au, gX, vi, Dd, OR, eq, Fc, aoz, BY, Tg, hd, CJ, py, db, LA, hD, nJ, d, je, hG, et, zR, aoF, aoW, dr, wW, bF, gk, wz, apv, rH, y, kS, hv, ng, aC, fR, hV, al, Qp, apF, Bc, pX, DO, K, aoV, apd, dF, eL, apl, aoo, apn, aB, aoY, apm, g, apB, aoS, iP, ou, AM, nF, U, dW, m, aov, QO, sw, OI, aw, NT, fN, qy, apt, apz, KL, ft, KH, CU, CN, aoX, ks, Og, YF, yR, apl, yP, it, aox, aoU, fM, aDE, jx, hz, j, il, pd, eA, er, XX, amg, lg, bq, aoP, kl, Y, r, aou, en, aoG, apg, iK, bN, QG, Dy, c, apu, hc, np, dy, fD, LS, gx, kh, id, aZ, bS, hF, iJ, vN, dP, fX, oj, io, apH, Yi, lR, xs, eH, cr, hp, iR, Nq, pu, hE, G, apr, pf, qx, aoM, XW, apj, oi, H, pk, Zm, hm, akV, aW, eM, iT, uJ, nw, zv, cZ, ajm, fr, KO, qN, ai, eW, ke, Bl, co, TK, h, dL, pj, hB, mY, Df, afu, Nr, aiB, iw, aq, apk, kp, Hu, mo, Ca, op, lx, aol, apa, Qu, hk, anC, hR, ajW, YH, bL, yk, hy, C, ze, aoC, Zp, ga, mP, nv, Vr, aoA, HC, aT, vH, dz, bK, an, af, aol, ak, qw, s, iU, oV, hf, f, Ta, sS, apq, wg, nM, Zg, lV, aoJ, ij, hS, Kd, Ke, aD, ha, Me, vm, Fb, OH, apC, mC, KL, av, F, ot, MS, ib, sO, zK, NS, nl, Od, fb, iL, apA, TC, qt, eZ, apb, Ej, aF, ek, es, aoR, bE, dJ, uR, aS, XO, X, Ex); #c1(BCL2A1) c2(NP_001108207) c3(bcl-2-related protein A1 isoform 2) c4(43154) c5(43155, 43156, 43157, 43158, 43159, 43160) c6( ) c7(bL, T, fM, FR, if, e, ht, iW, jh, aA, cz, fd, aj, j, ci, qN, Qp, q, J, ad, er, eA, apJ, qK, M, kH, aT, cy, h, dL, qD, ae, eL, je, aW, Y, r, aF, hf, eE, bz, ck, iM, eM, mg, cj, apv, cS, np, y, vQ); #c1(BCL2A1) c2(NP_004040) c3(bcl-2-related protein A1 isoform 1) c4(143063) c5(143064, 143065, 143066, 143067, 143068, 143069) c6( ) c7(bL, T, fM, fR, if, e, ht, iW, jh, aA, cz, fd, aj, j, ci, qN, Qp, q, J, ad, er, eA, apJ, qK, M, kH, aT, cy, h, dL, qD, ae, eL, je, aW, Y, r, aF, hf, eE, bz, ck, iM, eM, mg, cj, apv, cS, np, y, vQ); #c1(BCL2LI) c2(NP_001182) c3(bcl-2-like protein 1 isoform 2) c4( ) c5( ) c6( ) c7(bM, T, aC, fR, fd, bB, ci, aon, bv, xy, xE, aE, fm, K, hu, eL, cp, eG, aW, aoo, wf, hl, g, as, BF, BM, iT, U, cj, m, RX, ht, NT, aw, n, aj, fr, PJ, ft, jz, pe, Lv, cU, h, dL, KV, YT, A, CM, aq, pm, dw, iO, it, B, bL, fM, jp, aA, cR, hy, V, q, J, eA, oT, BJ, aT, kf, cy, bq, an, ae, af, eu, ak, s, nO, iU, oV, Y, apK, r, en, aom, t, ik, ck, c, gX, bt, hS, kh, id, iW, jh, dG, Fc, ad, av, aop, ib, iY, hP, d, ff, wc, eH, aF, iR, KM, bz, iq, es, pc, bE, dJ, gZ, Fk, y, X, BA, Tc); #c1(BCL2L1) c2(XP_011527268) c3(bcl-2-like protein 1 isoform X3) c4( ) c5(321863, 321864, 321865, 321866, 321867, 321868, 321869, 321870, 321871, 321872, 321873, 321874, 321875, 321876, 321877, 321878, 321879, 321880, 321881, 321882, 321883, 321884, 321885, 321886, 321887, 321888, 321889, 321890) c6(321891, 321892, 321893, 321894, 321895, 321896, 321897, 321898) c7(bM, T, aC, FR, fd, bB, ci, aon, bv, xy, xE, aE, fm, K, hu, eL, cp, eG, aW, aoo, wf, hl, g, as, eF, eM, iT, U, cj, m, RX, ht, NT, aw, n, aj, fr, PJ, ft, jz, pe, Lv, cU, h, dL, KV, YT, A, CM, aq, pm, dw, iO, it, B, bL, fM, jp, aA, cR, hy, V, q, J, eA, oT, BJ, aT, kf, cy, bq, an, ae, af, eu, ak, s, nO, iU, oV, Y, apk, r, en, aom, t, iK, ck, c, gX, bt, hS, kh, id, iW, jh, dG, Fc, ad, av, aop, ib, iY, hP, d, ff, wc, eH, aF, iR, KM, bz, iq, es, pc, bE, dJ, gZ, Fk, y, X, BA, Tc); #c1(BCL2L10) c2(NP_065129) c3(bcl-2-like protein 10) c4(204078) c5(204079, 204080, 204081, 204082, 204083, 204084) c6( ) c7(ck, T, ft, fM, aW, iW, qx, r, aT, aA, h, aF, fd, ci, bw, hF, bt, ae, ad); #c1(BCL2L11) c2(NP_001191036) c3(bcl-2-like protein 11 isoform (2) c4(80859) c5(80860, 80861, 80862, 80863, 80864) c6( ) c7(T, fM, FR, lT, sw, iW, jh, aA, bB, fd, hy, j, ci, fr, q, ad, x, av, ft, vJ, pX, aT, cU, K, pk, fb, ae, Yi, apL, gj, d, BN, s, B, iU, aW, Y, oV, eH, ff, r, hN, en, cl, g, t, eF, bz, cm, mK, ck, bF, eM, iA, aeB, bE, hL, cj, hR, k, m, bt, ne); #c1(BCL2LII) c2(NP_001191037) c3(bcl-2-like protein 11 isoform 13) c4(80865) c5(80866, 80867, 80868, 80869, 80870, 80871, 80872) c6( ) c7(T, fM, fR, lT, sw, iW, jh, aA, bB, fd, hy, j, ci, fr, q, ad, x, av, ft, vJ, pX, aT, cU, K, pk, fb, ae, Yi, apL, gj, d, BN, s, B, iU, aW, Y, oV, eH, ff, r, hN, en, cl, g, t, eF, bz, cm, mK, ck, bF, eM, iA, aeB, bE, hL, cj, hR, k, m, bt, ne); #c1(BCL2L11) c2(NP_001191038) c3(bcl-2-like protein II isoform 14) c4(80873) c5(80874, 80875, 80876, 80877, 80878, 80879) c6( ) c7(T, fM, FR, lT, sw, iW, jh, aA, bB, fd, hy, j, ci, fr, q, ad, x, av, ft, vJ, pX, aT, cU, K, pk, fb, ae, Yi, apL, gj, d, BN, s, B, iU, aW, Y, oV, eH, ff, r, hN, en, cl, g, t, eF, bz, cm, mK, ck, bF, eM, iA, aeB, bE, hL, cj, hR, k, m, bt, ne); #c1(BCL2LII) c2(NP_001191039) c3(bcl-2-like protein II isoform 15) c4(80880) c5(80881, 80882, 80883, 80884, 80885, 80886) c6( ) c7(T, fM, fR, lT, sw, iW, jh, aA, bB, fd, hy, j, ci, fr, q, ad, x, av, ft, vJ, pX, aT, cU, K, pk, fb, ae, Yi, apL, gj, d, BN, s, B, iU, aW, Y, oV, eH, ff, r, hN, en, cl, g, t, eF, bz, cm, mk, ck, bF, eM, iA, aeB, bE, hL, cj, hR, k, m, bt, ne); #c1(BCL2L11) c2(NP_001191040) c3(bcl-2-like protein II isoform 16) c4( ) c5(80887) c6( ) c7(T, fM, FR, lT, sw, iW, jh, aA, bB, fd, hy, j, ci, fr, q, ad, x, av, ft, vJ, pX, aT, cU, K, pk, fb, ae, Yi, apL, gj, d, BN, s, B, iU, aW, Y, oV, eH, ff, r, hN, en, cl, g, t, eF, bz, cm, mk, ck, bF, eM, iA, aeB, bE, hL, cj, hR, k, m, bt, ne); #c1(BCL2L11) c2(NP_001191041) c3(bcl-2-like protein 11 isoform (7) c4(80888) c5(80889, 80890, 80891, 80892, 80893, 80894) c6( ) c7(T, fM, fR, lT, sw, iW, jh, aA, bB, fd, hy, j, ci, fr, q, ad, x, av, ft, vJ, pX, aT, cU, K, pk, fb, ae, Yi, apL, gj, d, BN, s, B, iU, aW, Y, oV, eH, ff, r, hN, en, cl, g, t, eF, bz, cm, mk, ck, bF, eM, iA, aeB, bE, hL, cj, hR, k, m, bt, ne); #c1(BCL2LII) c2(NP_001191042) c3(bcl-2-like protein II isoform 18) c4( ) c5(80895, 80896, 80897, 80898, 80899, 80900) c6( ) c7(T, fM, FR, lT, sw, iW, jh, aA, bB, fd, hy, j, ci, fr, q, ad, x, av, ft, vJ, pX, aT, cU, K, pk, fb, ae, Yi, apl, gj, d, BN, s, B, iU, aW, Y, oV, eH, ff, r, hN, en, cl, g, t, eF, bz, cm, mk, ck, bF, eM, iA, aeB, bE, hL, cj, hR, k, m, bt, ne); #c1(BCL2L11) c2(NP_006529) c3(bcl-2-like protein II isoform 6) c4( ) c5(166039, 166040, 166041, 166042, 166043) c6( ) c7(T, fM, FR, lT, sw, iW, jh, aA, bB, fd, hy, j, ci, fr, q, ad, x, av, ft, vJ, pX, aT, cU, K, pk, fb, ae, Yi, apl, gj, d, BN, s, B, iU, aW, Y, oV, eH, ff, r, hN, en, cl, g, t, eF, bz, cm, mk, ck, bF, eM, iA, aeB, bE, hL, cj, hR, k, m, bt, ne); #c1(BCL2L11) c2(NP_619527) c3(bcl-2-like protein II isoform 1) c4( ) c5(236824, 236825, 236826, 236827, 236828) c6( ) c7(T, fM, FR, lT, sw, iW, jh, aA, bB, fd, hy, j, ci, fr, q, ad, x, av, ft, vJ, pX, aT, cU, K, pk, fb, ae, Yi, apL, gj, d, BN, s, B, iU, aW, Y, oV, eH, ff, r, hN, en, cl, g, t, eF, bz, cm, mK, ck, bF, eM, iA, aeB, bE, hL, cj, hR, k, m, bt, ne); #c1 (BCL2L11) c2(NP_619530) c3(bcl-2-like protein II isoform 4) c4(236829) c5(236830, 236831, 236832, 236833, 236834) c6( ) c7(T, fM, FR, lT, sw, iW, jh, aA, bB, fd, hy, j, ci, fr, q, ad, x, av, ft, vJ, pX, aT, cU, K, pk, fb, ae, Yi, apL, gj, d, BN, s, B, iU, aW, Y, oV, eH, ff, r, hN, en, cl, g, t, eF, bz, cm, mK, ck, bF, eM, iA, aeB, bE, hL, cj, hR, k, m, bt, ne); #c1(BCL2L11) c2(NP_619531) c3(bcl-2-like protein II isoform 5) c4(236835) c5(236836) c6( ) c7(T, fM, FR, lT, sw, iW, jh, aA, bB, fd, hy, j, ci, fr, q, ad, x, av, ft, vJ, pX, aT, cU, K, pk, fb, ae, Yi, apL, gj, d, BN, s, B, iU, aW, Y, oV, eH, ff, r, hN, en, cl, g, t, eF, bz, cm, mK, ck, bF, eM, iA, aeB, bE, hL, cj, hR, k, m, bt, ne); #c1(BCL2LII) c2(NP_619532) c3(bcl-2-like protein 11 isoform 7) c4( ) c5(236837, 236838, 236839, 236840, 236841, 236842, 236843, 236844, 236845, 236846) c6( ) c7(T, fM, fR, lT, sw, iW, jh, aA, bB, fd, hy, j, ci, fr, q, ad, x, av, ft, vJ, pX, aT, cU, K, pk, fb, ae, Yi, apL, gj, d, BN, s, B, iU, aW, Y, oV, eH, ff, r, hN, en, cl, g, t, eF, bz, cm, mk, ck, bF, eM, iA, aeB, bE, hL, cj, hR, k, m, bt, ne); #c1(BCL2L11) c2(NP_996885) c3(bcl-2-like protein II isoform 9) c4( ) c5(264805, 264806, 264807, 264808, 264809) c6( ) c7(T, fM, FR, lT, sw, iW, jh, aA, bB, fd, hy, j, ci, fr, q, ad, x, av, ft, vJ, pX, aT, cU, K, pk, fb, ae, Yi, apL, gj, d, BN, s, B, iU, aW, Y, oV, eH, ff, r, hN, en, cl, g, t, eF, bz, cm, mk, ck, bF, eM, iA, aeB, bE, hL, cj, hR, k, m, bt, ne); #c1(BCL2L11) c2(XP_005263607) c3(bcl-2-like protein II isoform X1) c4(278484) c5( ) c6( ) c7(T, fM, FR, lT, sw, iW, jh, aA, bB, fd, hy, j, ci, fr, q, ad, x, av, ft, vJ, pX, aT, cU, K, pk, fb, ae, Yi, apl, gj, d, BN, s, B, iU, aW, Y, oV, eH, ff, r, hN, en, cl, g, t, eF, bz, cm, mK, ck, bF, eM, iA, aeB, bE, hL, cj, hR, k, m, bt, ne); #c1(BCL2L11) c2(XP_005263608) c3(bcl-2-like protein II isoform X2) c4(278485) c5(278486) c6( ) c7(T, fM, FR, lT, sw, iW, jh, aA, bB, fd, hy, j, ci, fr, q, ad, x, av, ft, vJ, pX, aT, cU, K, pk, fb, ae, Yi, apL, gj, d, BN, s, B, iU, aW, Y, oV, eH, ff, r, hN, en, cl, g, t, eF, bz, cm, mk, ck, bF, eM, iA, aeB, bE, hL, cj, hR, k, m, bt, ne); #c1(BCL2LII) c2(XP_005263610) c3(bcl-2-like protein 11 isoform X6) c4(278487) c5(278488) c6( ) c7(T, fM, FR, lT, sw, iW, jh, aA, bB, fd, hy, j, ci, fr, q, ad, x, av, ft, vJ, pX, aT, cU, K, pk, fb, ae, Yi, apl, gj, d, BN, s, B, iU, aW, Y, oV, eH, ff, r, hN, en, cl, g, t, eF, bz, cm, mK, ck, bF, eM, iA, aeB, bE, hL, cj, hR, k, m, bt, ne); #c1(BCL2LII) c2(XP_005263611) c3(bcl-2-like protein 11 isoform X7) c4( ) c5(278489, 278490) c6( ) c7(T, fM, fR, lT, sw, iW, jh, aA, bB, fd, hy, j, ci, fr, q, ad, x, av, ft, vJ, pX, aT, cU, K, pk, fb, ae, Yi, apL, gj, d, BN, s, B, iU, aW, Y, oV, eH, ff, r, hN, en, cl, g, t, eF, bz, cm, mk, ck, bF, eM, iA, aeB, bE, hL, cj, hR, k, m, bt, ne); #c1(BCL2L11) c2(XP_005263616) c3(bcl-2-like protein II isoform X12) c4( ) c5(278491) c6( ) c7(T, fM, FR, lT, sw, iW, jh, aA, bB, fd, hy, j, ci, fr, q, ad, x, av, ft, vJ, pX, aT, cU, K, pk, fb, ae, Yi, apL, gj, d, BN, s, B, iU, aW, Y, oV, eH, ff, r, hN, en, cl, g, t, eF, bz, cm, mK, ck, bF, eM, iA, aeB, bE, hL, cj, hR, k, m, bt, ne); #c1(BCL2L11) c2(XP_005263618) c3(bcl-2-like protein II isoform X14) c4( ) c5(278492, 278493, 278494, 278495, 278496, 278497) c6( ) c7(T, fM, fR, lT, sw, iW, jh, aA, bB, fd, hy, j, ci, fr, q, ad, x, av, ft, vJ, pX, aT, cU, K, pk, fb, ae, Yi, apL, gj, d, BN, s, B, iU, aW, Y, oV, eH, ff, r, hN, en, cl, g, t, eF, bz, cm, mk, ck, bF, eM, iA, aeB, bE, hL, cj, hR, k, m, bt, ne); #c1(BCL2L(1) c2(XP_011508765) c3(bcl-2-like protein II isoform X8) c4(299366, 299367) c5( ) c6( ) c7(T, fM, FR, lT, sw, iW, jh, aA, bB, fd, hy, j, ci, fr, q, ad, x, av, ft, vJ, pX, aT, cU, K, pk, fb, ae, Yi, apl, gj, d, BN, s, B, iU, aW, Y, oV, eH, ff, r, hN, en, cl, g, t, eF, bz, cm, mk, ck, bF, eM, iA, aeB, bE, hL, cj, hR, k, m, bt, ne); #c1 (BCL2L11) c2(XP_01508766) c3(bcl-2-like protein II isoform X11) c4(299368) c5(299369) c6( ) c7(T, fM, fR, lT, sw, iW, jh, aA, bB, fd, hy, j, ci, fr, q, ad, x, av, ft, vJ, pX, aT, cU, K, pk, fb, ae, Yi, apL, gj, d, BN, s, B, iU, aW, Y, oV, eH, ff, r, hN, en, cl, g, t, eF, bz, cm, mk, ck, bF, eM, iA, aeB, bE, hL, cj, hR, k, m, bt, ne); #c1(BCL2L11) c2(XP_011508767) c3(bcl-2-like protein 11 isoform X13) c4(299370) c5( ) c6( ) c7(T, fM, FR, lT, sw, iW, jh, aA, bB, fd, hy, j, ci, fr, q, ad, x, av, ft, vJ, pX, aT, cU, K, pk, fb, ae, Yi, apl, gj, d, BN, s, B, iU, aW, Y, oV, eH, ff, r, hN, en, cl, g, t, eF, bz, cm, mk, ck, bF, eM, iA, aeB, bE, hL, cj, hR, k, m, bt, ne); #c1 (BCL2L12) c2(NP_001035758) c3(bcl-2-like protein 12 isoform 3) c4(33049) c5(33050, 33051, 33052, 33053, 33054, 33055, 33056) c6( ) c7(gk, T, d, JI, aW, bE, iW, al, jx, aT, cj, n, aA, dL, bB, m, eA, aF, t, eL, X); #c1(BCL2L12) c2(NP_001269446) c3(bcl-2-like protein (2 isoform 2) c4(101683) c5(101684, 101685, 101686, 101687, 101688) c6( ) c7(gk, T, d, JI, aW, bE, iW, al, jx, aT, cj, n, aA, dL, bB, m, eA, aF, t, eL, X); #c1(BCL2L12) c2(NP_619580) c3(bcl-2-like protein 12 isoform 1) c4(236887) c5(236888, 236889, 236890, 236891, 236892, 236893) c6( ) c7(gk, T, d, JI, aW, bE, iW, al, jx, aT, cj, n, aA, dL, bB, m, eA, aF, t, eL, X); #c1(BCL2L13) c2(NP_001257656) c3(bcl-2-like protein 13 isoform c) c4( ) c5(95549, 95550) c6( ) c7(aq, iW, B, ad); #c1(BCL2L13) c2(NP_001257660) c3(bcl-2-like protein 13 isoform e) c4( ) c5(95551, 95552, 95553, 95554, 95555, 95556, 95557, 95558, 95559, 95560, 95561, 95562, 95563, 95564, 95565) c6( ) c7(aq, iW, B, ad); #c1(BCL2L13) c2(NP_001257663) c3(bcl-2-like protein 13 isoform h) c4(95566, 95567) c5(95568, 95569, 95570, 95571, 95572) c6( ) c7(aq, iW, B, ad); #c1(BCL2L13) c2(NP_001257664) c3(bcl-2-like protein 13 isoform i) c4( ) c5(95573, 95574, 95575, 95576, 95577) c6( ) c7(aq, iW, B, ad); #c1(BCL2L13) c2(XP_011544423) c3(bcl-2-like protein 13 isoform X1) c4(343513, 343514, 343515, 343516) c5(343517, 343518, 343519, 343520, 343521, 343522, 343523, 343524, 343525, 343526, 343527, 343528, 343529, 343530, 343531, 343532) c6( ) c7(aq, iW, B, ad); #c1(BCL2L13) c2(XP_011544424) c3(bcl-2-like protein 13 isoform X4) c4( ) c5(343533) c6( ) c7(aq, iW, B, ad); #c1(BCL2L14) c2(NP_110393) c3(apoptosis facilitator Bcl-2-like protein 14 isoform 2) c4(219996) c5(219997, 219998, 219999, 220000, 220001, 220002, 220003, 220004, 220005, 220006, 220007, 220008, 220009, 220010, 220011, 220012, 220013) c6( ) c7(cF, id, aW, eL, iW, aT, dL, ae, ad); #c1(BCL2L14) c2(XP_011519151) c3(apoptosis facilitator Bcl-2-like protein 14 isoform X1) c4(313741, 313742, 313743) c5(313744, 313745, 313746, 313747, 313748, 313749, 313750, 313751, 313752, 313753, 313754) c6( ) c7(cF, id, aW, eL, iW, aT, dL, ae, ad); #c1(BCL2L2) c2(NP_004041) c3(bcl-2-like protein 2) c4(143070, 143071) c5(143072, 143073, 143074, 143075, 143076, 143077, 143078, 143079, 143080, 143081, 143082, 143083, 143084, 143085, 143086, 143087, 143088, 143089, 143090, 143091, 143092, 143093, 143094, 143095, 143096, 143097, 143098, 143099, 143100, 143101) c6( ) c7(T, gU, fM, iW, hy, V, eA, ft, aT, vH, ne, af, d, BN, ND, iU, B, aW, cm, ck, eM, iu); #c1(BCL2L2-PABPN1) c2(NP_001186793) c3(BCL2L2-PABPN1 protein) c4(78596, 78597, 78598) c5( ) c6( ) c7(ck, iu, T, ft, d, eM, aW, aT, cm, V, eA); #c1(BCL3) c2(XP_011525499) c3(B-cell lymphoma 3 protein isoform X1) c4(319891) c5(319892, 319893, 319894, 319895, 319896, 319897, 319898, 319899) c6( ) c7(T, fM, iW, e, eX, jh, ad, Na, aT, KH, h, dz, eV, Nc, ji, ae, apN, bc, je, aW, Y, fe, iV, eE, t, dR, ck, apM, iM, cj, m); #c1(BCL3) c2(XP_011525501) c3(B-cell lymphoma 3 protein isoform X2) c4(319900) c5(319901, 319902, 319903, 319904, 319905, 319906) c6( ) c7(T, fM, iW, e, ex, jh, ad, Na, aT, kH, h, dz, eV, Nc, ji, ae, apN, bc, je, aW, Y, fe, iV, eE, t, dR, ck, apM, iM, cj, m); #c1(BCL6) c2(NP_001124317) c3(B-cell lymphoma 6 protein isoform 1) c4(48537) c5(48538, 48539, 48540, 48541, 48542, 48543, 48544, 48545, 48546, 48547) c6( ) c7(gx, T, os, fM, dA, ajm, id, iW, jx, eX, oA, aA, Bm, fd, oZ, apR, jh, ci, hF, iJ, ip, x, apS, F, apl, hW, zK, aT, io, h, eV, hn, hP, ox, jj, pk, fb, jJ, Df, aol, Fp, Uf, d, iU, kS, aW, Y, eH, aph, Of, Og, aF, aoD, Ca, eF, f, fs, op, Wt, ck, kn, ik, bP, iQ, apO, dB, Zg, vA, cj, at, SZ, k, ij, zv, i, y, apE, apP, jr); #c1(BCL6) c2(NP_001128210) c3(B-cell lymphoma 6 protein isoform 2) c4(49692, 49693, 49694) c5(49695, 49696, 49697, 49698, 49699, 49700, 49701, 49702, 49703, 49704, 49705, 49706, 49707, 49708, 49709, 49710, 49711, 49712) c6( ) c7(gx, T, os, fM, dA, ajm, id, iW, jx, eX, oA, aA, Bm, fd, oZ, apR, jh, ci, hF, iJ, ip, x, apS, F, apl, hW, zK, aT, io, h, eV, hn, hP, ox, jj, pk, fb, jJ, Df, aol, Fp, Uf, d, iU, kS, aW, Y, eH, aph, Of, Og, af, aoD, Ca, eF, f, fs, op, Wt, ck, kn, ik, bP, iQ, apO, dB, Zg, vA, cj, at, SZ, k, ij, zv, i, y, apE, apP, jr); #c1(BCL6B) c2(NP_862827) c3(B-cell CLL/lymphoma 6 member B protein) c4(255789, 255790) c5(255791, 255792, 255793, 255794, 255795, 255796, 255797, 255798, 255799, 255800, 255801, 255802, 255803) c6( ) c7(ds, T, aF, aA); #c1(BCL7A) c2(NP_001019979) c3(B-cell CLL/lymphoma 7 protein family member A isoform b) c4(27979) c5(27980, 27981, 27982, 27983, 27984, 27985) c6( ) c7(apT, pk, iW, fM, xR); #c1(BCL7A) c2(NP_066273) c3(B-cell CLL/lymphoma 7 protein family member A isoform a) c4(206891) c5(206892, 206893, 206894, 206895, 206896, 206897) c6( ) c7(apT, pk, iW, fM, xR); #c1(BCL7B) c2(NP_001184/73) c3(B-cell CLL/lymphoma 7 protein family member B isoform 2) c4(75506) c5(75507, 75508, 75509, 75510, 75511, 75512) c6( ) c7(bL, g, ei); #c1(BCL7B) c2(NP_001698) c3(B-cell CLL/lymphoma 7 protein family member B isoform 1) c4(120113) c5(120114, 120115, 120116, 120117, (20118, (2019) c6( ) c7(bL, g, ei); #c1(BCL7C) c2(NP_001273455) c3(B-cell CLL/lymphoma 7 protein family member C isoform 1) c4(104951) c5(104952, 104953, 104954, 104955, 104956, 104957) c6( ) c7(fM); #c1(BCL7C) c2(NP_004756) c3(B-cell CLL/lymphoma 7 protein family member C isoform 2) c4(149343) c5(149344, 149345, 149346, 149347, 149348, 149349, 149350) c6( ) c7(fM); #c1(BCL9L) c2(NP_872363) c3(B-cell CLL/lymphoma 9-like protein) c4(256223, 256224, 256225) c5(256226, 256227, 256228, 256229, 256230, 256231, 256232, 256233, 256234, 256235, 256236, 256237, 256238, 256239, 256240, 256241, 256242, 256243) c6( ) c7(ac, s, X, ha, aj, lQ, cd, Qe); #c1(BCLAF1) c2(NP_001070908) c3(bcl-2-associated transcription factor 1 isoform 2) c4(34905) c5(34906, 34907, 34908, 34909, 34910, 34911, 34912, 34913, 34914, 34915) c6( ) c7(iW, ft, Fw); #c1(BCLAF1) c2(NP_001070909) c3(bcl-2-associated transcription factor 1 isoform 3) c4(34916, 34917) c5(34918, 34919, 34920, 34921, 34922, 34923) c6( ) c7(iW, ft, Fw); #c1(BCLAF1) c2(NP_001287967) c3(bcl-2-associated transcription factor 1 isoform 4) c4(113279) c5(113280, 113281, 113282, 113283, 113284, 113285, 113286, 113287, 113288, 113289, 113290, 113291) c6( ) c7(iW, ft, Fw); #c1(BCLAF1) c2(NP_055554) c3(bcl-2-associated transcription factor 1 isoform 1) c4(181914) c5( ) c6( ) c7(iW, ft, Fw); #c1(BCLAF1) c2(XP_005267296) c3(bcl-2-associated transcription factor 1 isoform X3) c4( ) c5(280882, 280883, 280884, 280885, 280886, 280887, 280888, 280889) c6( ) c7(iW, ft, Fw); #c1(BCO1) c2(NP_059125) c3(beta,beta-carotene 15,15′-monooxygenase) c4(193149, 193150) c5(193151, 193152, 193153, 193154, 193155, 193156, 193157, 193158, 193159, 193160, 193161, 193162) c6( ) c7(bL, T, lR, W, bE, aW, aT, bB, yy, h, apU, ae, q, ad); #c1(BCO2) c2(NP_001243326) c3(beta,beta-carotene 9′, 10′-oxygenase isoform c) c4(90410) c5(90411, 90412, 90413, 90414, 90415) c6( ) c7(K); #c1(BCO2) c2(NP_114144) c3(beta,beta-carotene 9′, 10′-oxygenase isoform a) c4(222323, 222324) c5(222325, 222326, 222327, 222328, 222329, 222330, 222331, 222332, 222333, 222334, 222335, 222336, 222337, 222338, 222339, 222340, 222341, 222342, 222343, 222344, 222345) c6( ) c7(K); #c1(BCOR) c2(NP_001116855) c3(BCL-6 corepressor isoform a) c4(44006, 44007, 44008) c5(44009, 44010, 44011, 44012, 44013, 44014, 44015, 44016, 44017, 44018) c6( ) c7(ka, T, bs, br, qK, KN, jj, dx, apV, iF, jf, kj, gm, eF, bw, ck, kn, bP, ki, apW, cj, Km, Th, bt, B); #c1(BCOR) c2(XP_005272677) c3(BCL-6 corepressor isoform X1) c4(284122, 284123) c5(284124, 284125, 284126, 284127, 284128, 284129, 284130, 284131, 284132, 284133, 284134, 284135, 284136, 284137, 284138, 284139, 284140, 284141, 284142) c6( ) c7(ka, T, bs, br, qK, KN, jj, dx, apV, iF, jf, kj, gm, eF, bw, ck, kn, bP, ki, apW, cj, Km, Th, bt, B); #c1(BCOR) c2(XP_011542233) c3(BCL-6 corepressor isoform X2) c4(340727) c5(340728, 340729, 340730, 340731, 340732, 340733, 340734, 340735, 340736, 340737, 340738) c6( ) c7(ka, T, bs, br, qK, KN, jj, dx, apV, iF, jf, kj, gm, eF, bw, ck, kn, bP, ki, apW, cj, Km, Th, bt, B); #c1(BCORL1) c2(NP_068765) c3(BCL-6 corepressor-like protein 1) c4(210275) c5(210276, 210277, 210278, 210279, 210280, 210281, 210282, 210283, 210284, 210285) c6( ) c7(ka, T, bP, aW, ki, qK, aT, bs, jj, bw, bt, B, br); #c1(BCORL1) c2(XP_005262513) c3(BCL-6 corepressor-like protein 1 isoform X3) c4(277976, 277977) c5(277978, 277979, 277980, 277981, 277982, 277983, 277984, 277985, 277986, 277987, 277988, 277989, 277990, 277991) c6( ) c7(ka, T, bP, aW, ki, qK, aT, bs, jj, bw, bt, B, br); #c1(BCORL1) c2(XP_006724842) c3(BCL-6 corepressor-like protein 1 isoform X2) c4(296452) c5( ) c6( ) c7(ka, T, bP, aW, ki, qK, aT, bs, jj, bw, bt, B, br); #c1(BCR) c2(NP_004318) c3(breakpoint cluster region protein isoform 1) c4(145117, 145118, 145119, 145120, 145121, 145122) c5(145123, 145124, 145125, 145126, 145127, 145128, 145129, 145130, 145131, 145132, 145133, 145134) c6( ) c7(T, os, qx, al, bO, fd, ip, x, OG, K, or, mH, pr, fc, Ku, bw, Qt, ou, iT, vA, cj, k, R, qm, gp, RX, Or, DE, n, ko, oY, br, oC, KH, eV, hn, dL, ox, pi, dx, fe, pm, apX, op, Wt, bP, at, apY, pg, B, fM, dA, eX, oD, fZ, ol, apZ, v, q, er, zn, oT, fh, dz, oF, bK, jj, af, jf, iU, Y, r, hf, ho, ck, ov, iM, oX, bt, Ze, vi, ow, DA, bs, jh, oZ, oH, hF, ad, afb, fx, oW, iY, fb, pq, iL, aqa, d, je, et, eZ, kD, bz, eU, Ew, pc, cP, y, kS, jr); #c1(BCR) c2(NP_067585) c3(breakpoint cluster region protein isoform 2) c4(209003) c5(209004, 209005, 209006, 209007, 209008, 209009, 209010, 209011, 209012, 209013, 209014, 209015, 209016, 209017, 209018, 209019, 209020, 209021, 209022, 209023, 209024, 209025, 209026, 209027, 209028, 209029, 209030, 209031, 209032, 209033) c6( ) c7(T, os, qx, al, bO, fd, ip, x, OG, K, or, mH, pr, fc, Ku, bw, Qt, ou, iT, vA, cj, k, R, qm, gp, RX, Or, DE, n, ko, oY, br, oC, kH, eV, hn, dl, ox, pi, dx, fe, pm, apX, op, Wt, bP, at, apY, pg, B, fM, dA, eX, oD, fZ, ol, apZ, v, q, er, zn, oT, fh, dz, oF, bK, jj, af, jf, iU, Y, r, hf, ho, ck, ov, iM, oX, bt, Ze, vi, ow, oA, bs, jh, oZ, OH, hF, ad, afb, fx, oW, iY, fb, pq, iL, aga, d, je, et, eZ, KD, bz, eU, Ew, pc, cP, y, kS, jr); #c1(BCS1L) c2(NP_001073335) c3 (mitochondrial chaperone BCS1) c4(35896, 35897, 35898, 35899) c5(35900, 35901, 35902, 35903, 35904, 35905, 35906, 35907, 35908, 35909, 35910, 35911, 35912, 35913, 35914, 35915, 35916, 35917, 35918, 35919, 35920, 35921, 35922, 35923, 35924, 35925, 35926, 35927, 35928, 35929, 35930, 35931, 35932, 35933, 35934, 35935, 35936, 35937, 35938, 35939, 35940, 35941, 35942, 35943, 35944, 35945, 35946, 35947, 35948, 35949, 35950, 35951) c6( ) c7(T, DM, aqd, aqf, aqb, eA, hb, h, K, aqe, aH, ajU, KE, ev, cm, kC, ek, cT, SE, aqc, mG, bt); #c1(BDH1) c2(NP_976059) c3(D-beta-hydroxybutyrate dehydrogenase, mitochondrial precursor) c4(262904, 262905, 262906, 262907) c5(262908, 262909, 262910, 262911, 262912, 262913, 262914, 262915, 262916, 262917, 262918, 262919, 262920, 262921, 262922, 262923, 262924, 262925, 262926, 262927, 262928, 262929, 262930, 262931, 262932, 262933) c6( ) c7(aW, hr, aT, fQ, bq, Kp, eA); #c1(BDH1) c2(XP_011511370) c3(D-beta-hydroxybutyrate dehydrogenase, mitochondrial isoform X3) c4( ) c5(301943, 301944, 301945, 301946, 301947, 301948) c6( ) c7(aW, hr, aT, fQ, bq, Kp, eA); #c1(BDH2) c2(XP_005263197) c3(3-hydroxybutyrate dehydrogenase type 2 isoform X3) c4( ) c5(278346, 278347, 278348, 278349, 278350, 278351, 278352, 278353, 278354, 278355, 278356, 278357) c6( ) c7(d, yw, m, as, Rc, t, bt); #c1(BDH2) c2(XP_011530429) c3(3-hydroxybutyrate dehydrogenase type 2 isoform X2) c4(326293, 326294) c5( ) c6( ) c7(d, yw, m, as, Rc, t, bt); #c1(BDH2) c2(XP_011530430) c3(3-hydroxybutyrate dehydrogenase type 2 isoform X1) c4(326295) c5(326296, 326297, 326298, 326299, 326300, 326301) c6( ) c7(d, yw, m, as, Rc, t, bt); #c1 (BDKRB1) c2(NP_000701) c3(B1 bradykinin receptor) c4(14731) c5(14732, 14733, 14734, 14735, 14736, 14737) c6( ) c7(bM, T, eR, e, ha, My, aw, aA, GP, cz, aj, v, j, eb, il, aY, V, eA, iu, uT, aT, ey, K, bq, he, dl, uX, s, ES, aW, sm, fe, aF, iV, qa, xZ, vy, JM, dy, eo, st); #c1(BDKRB2) c2(NP_000614) c3(B2 bradykinin receptor) c4(13780, 13781) c5(13782, 13783, 13784, 13785, 13786, 13787, 13788, 13789, 13790, 13791, 13792, 13793, 13794, 13795, 13796, 13797, 13798, 13799, 13800, 13801) c6( ) c7(bL, bM, T, Fj, eR, oe, ha, e, uG, aA, GP, cz, My, aj, j, v, bO, ww, il, aY, eA, aw, cc, ql, bk, bq, he, sR, dl, af, aa, UO, tb, sm, fe, aF, uC, qa, ar, RJ, vy, at, lC, iu, y, qc); #c1(BDNF) c2(NP_001137280) c3(brain-derived neurotrophic factor isoform a preproprotein) c4( ) c5(55198, 55199, 55200, 55201, 55202, 55203, 55204, 55205, 55206, 55207, 55208, 55209, 55210, 55211, 55212, 55213, 55214, 55215, 55216, 55217, 55218, 55219, 55220, 55221, 55222, 55223, 55224, 55225, 55226, 55227, 55228, 55229, 55230, 55231, 55232, 55233, 55234, 55235, 55236, 55237, 55238, 55239, 55240, 55241) c6( ) c7(ID, tJ, T, uk, bM, Fv, xo, ago, e, lr, bO, fd, gf, HH, aR, ku, il, fm, bW, pX, Fo, Qk, M, iX, aU, abj, K, qM, aqD, oi, H, xa, aqn, KC, ZH, amy, LC, aqB, aW, wf, fc, abn, vt, Ku, Eg, as, eE, cm, mZ, yX, aqr, bb, eM, aqz, nB, ZT, U, vy, sv, nw, uJ, m, act, Zo, cZ, FW, gU, DI, My, fN, aqy, aj, ww, ct, nA, aqL, Vs, ke, Mo, ft, Hx, alq, aqJ, rl, Lv, lG, By, aqK, h, eV, dd, mV, mY, he, dx, aqi, mU, abf, aqg, PT, CN, A, aqA, dw, cM, fq, aqG, Jh, cv, mX, bJ, hq, ok, amt, aqp, cA, bP, dB, Ei, Be, hL, vL, at, JM, aqm, eo, lB, cB, mT, WI, B, L, bL, qc, it, jd, fM, iH, fG, lH, He, qQ, eX, aA, cR, mS, ti, cz, j, v, bX, aql, il, GC, V, q, J, aqs, aqF, er, eA, PW, aqt, di, aqN, yw, HJ, nv, WY, hb, uT, rS, aql, HC, aT, kf, cy, bK, an, rE, ae, apN, lr, aqC, af, eu, ak, aqv, iF, jf, Y, yL, abi, ahe, r, cu, aaE, HL, en, cl, t, f, ik, FT, tl, ck, Qi, pH, cT, bN, ac, c, rR, yW, wO, mG, aqH, Da, JH, EY, fD, gT, hl, kh, qX, VR, id, wi, agh, iW, ha, jN, Gd, bS, aqu, fu, uu, qi, hF, ad, BY, dP, HF, aqq, hd, ot, mW, sO, aqk, aqx, xp, ahK, nJ, na, aqE, d, VX, aqj, tb, aaQ, aog, et, Gv, ff, gC, zR, eZ, gl, aF, Z, FX, jb, bz, dr, DF, ek, Hw, EW, aqM, z, dJ, aaG, MJ, cP, ei, pK, lC, aqw, xg, iu, y); #c1(BDNF) c2(NP_001137281) c3(brain-derived neurotrophic factor isoform d preproprotein) c4( ) c5(55242, 55243, 55244) c6( ) c7(ID, tJ, T, uk, bM, Fv, xo, ago, e, lr, bO, fd, gf, HH, aR, ku, il, fm, bW, pX, Fo, Qk, M, iX, aU, abj, K, qM, aqD, oi, H, xa, aqn, KC, ZH, amy, LC, aqB, aW, wf, fc, abn, vt, Ku, Eg, as, eE, cm, mZ, yX, aqr, bb, eM, aqz, nB, ZT, U, vy, sv, nw, uJ, m, act, Zo, cZ, FW, gU, DI, My, fN, aqy, aj, ww, ct, nA, aqL, Vs, ke, Mo, ft, Hx, alq, aqJ, rl, Lv, lG, By, aqK, h, eV, dd, mV, mY, he, dx, aqi, mU, abf, aqg, PT, CN, A, aqA, dw, cM, fq, aqG, Jh, cv, mX, bJ, hq, ok, amt, aqp, cA, bP, dB, Ei, Be, hL, vL, at, JM, aqm, eo, lB, cB, mT, WI, B, L, bL, qc, it, jd, fM, iH, fG, lH, He, qQ, eX, aA, cR, mS, ti, cz, j, v, bX, aql, il, GC, V, q, J, aqs, aqF, er, eA, PW, aqt, di, aqN, yw, HJ, nv, WY, hb, uT, rS, aql, HC, aT, kf, cy, bK, an, rE, ae, apN, lr, aqC, af, eu, ak, aqv, iF, jf, Y, yL, abi, ahe, r, cu, aaE, HL, en, cl, t, f, ik, FT, tl, ck, Qi, pH, cT, bN, ac, c, rR, yW, wl, mG, aqH, Da, JH, EY, fD, gT, hl, kh, qX, VR, id, wi, aqh, iW, ha, jN, Gd, bS, aqu, fu, uu, qi, hF, ad, BY, dP, HF, aqq, hd, ot, mW, sO, aqk, aqx, xp, ahK, nJ, na, aqE, d, VX, aqj, tb, aaQ, aog, et, Gv, ff, gC, zR, eZ, gl, aF, Z, FX, jb, bz, dr, DF, eK, Hw, EW, aqM, z, dJ, aaG, MJ, cP, ei, pK, lC, aqw, xg, iu, y); #c1(BDNF) c2(NP_001137282) c3(brain-derived neurotrophic factor isoform e) c4(55245, 55246) c5(55247, 55248, 55249, 55250, 55251, 55252, 55253, 55254, 55255, 55256, 55257, 55258) c6( ) c7(ID, tJ, T, uk, bM, Fv, xo, aqo, e, lr, bO, fd, gf, HH, aR, ku, il, fm, bW, pX, Fo, Qk, M, iX, aU, abj, K, qM, aqD, oi, H, xa, aqn, KC, ZH, amy, LC, aqB, aW, wf, fc, abn, vt, Ku, Eg, as, eE, cm, mZ, yX, aqr, bb, eM, aqz, nB, ZT, U, vy, sv, nw, uJ, m, act, Zo, cZ, FW, gU, Ol, My, fN, aqy, aj, ww, ct, nA, aqL, Vs, ke, Mo, ft, Hx, alq, aqJ, rl, Lv, lG, By, aqK, h, eV, dd, mV, mY, he, dx, aqi, mU, abf, aqg, PT, CN, A, aqA, dw, cM, fq, aqG, Jh, cv, mX, bJ, hq, ok, amt, aqp, cA, bP, dB, Ei, Be, hl, vl, at, JM, aqm, eo, lB, cB, mT, WI, B, L, bL, qc, it, jd, FM, iH, fG, IH, He, qQ, eX, aA, cR, mS, ti, cz, j, v, bX, aql, il, GC, V, q, J, aqs, aqF, er, eA, PW, aqt, di, aqN, yw, HJ, nv, WY, hb, uT, rS, aql, HC, aT, kf, cy, bk, an, rE, ae, apN, lr, aqC, af, eu, ak, aqv, iF, jf, Y, yL, abi, ahe, r, cu, aaE, HL, en, cl, t, f, ik, FT, tl, ck, Qi, pH, cT, bN, ac, c, rR, yW, w, mG, aqH, Da, JH, EY, fD, gT, hl, kh, qX, VR, id, wi, agh, iW, ha, jN, Gd, bS, aqu, fu, uu, qi, hF, ad, BY, dP, HF, aqq, hd, ot, mW, s, aqk, aqx, xp, ahK, nJ, na, aqE, d, VX, aqj, tb, aaQ, aog, et, Gv, ff, gC, zR, eZ, gl, aF, Z, FX, jb, bz, dr, DF, eK, Hw, EW, aqM, z, dJ, aaG, MJ, cP, ei, pK, lC, aqw, xg, iu, y); #c1(BDNF) c2(NP_733927) c3(brain-derived neurotrophic factor isoform b preproprotein) c4(247246, 247247) c5(247248, 247249, 247250, 247251, 247252, 247253, 247254, 247255, 247256, 247257, 247258, 247259, 247260, 247261, 247262) c6( ) c7(ID, tJ, T, uK, bM, Fv, xo, ago, e, lr, bO, fd, gf, HH, aR, ku, il, fm, bW, pX, Fo, Qk, M, iX, aU, abj, K, qM, aqD, oi, H, xa, aqn, KC, ZH, amy, LC, aqB, aW, wf, fc, abn, vt, Ku, Eg, as, eE, cm, mZ, yX, aqr, bb, eM, aqz, nB, ZT, U, vy, sv, nw, uJ, m, act, Zo, cZ, FW, gU, OI, My, fN, aqy, aj, ww, ct, nA, aqL, Vs, ke, Mo, ft, Hx, alq, aqJ, d, Lv, lG, By, aqK, h, eV, dd, mV, mY, he, dx, aqi, mU, abf, aqg, PT, CN, A, aqA, dw, cM, fq, agG, Jh, cv, mX, bJ, hq, ok, amt, aqp, cA, bP, dB, Ei, Be, hl, vL, at, JM, aqm, eo, lB, cB, mT, WI, B, L, bL, qc, it, jd, fM, iH, fG, lH, He, qQ, eX, aA, cR, mS, ti, cz, j, v, bX, aql, il, GC, V, q, J, aqs, aqF, er, eA, PW, aqt, di, aqN, yw, HJ, nv, WY, hb, uT, rS, aql, HC, aT, kf, cy, bK, an, rE, ae, apN, lr, aqC, af, eu, ak, aqv, if, jf, Y, yL, abi, ahe, r, cu, aaE, HL, en, cl, t, f, iK, FT, tl, ck, Qi, pH, cT, bN, ac, c, rR, yW, wl, mG, aqH, Da, JH, EY, fD, gT, hl, kh, qX, VR, id, wi, agh, iW, ha, jN, Gd, bS, aqu, fu, uu, qi, hF, ad, BY, dP, HF, aqq, hd, ot, mW, sO, ak, aqx, xp, ahK, nJ, na, aqE, d, VX, aqj, tb, aaQ, aog, et, Gv, ff, gC, zR, eZ, gl, aF, Z, FX, jb, bz, dr, DF, ek, Hw, EW, aqM, z, dJ, aaG, MJ, cP, ei, pk, lC, aqw, xg, iu, y); #c1(BDNF) c2(NP_733930) c3(brain-derived neurotrophic factor isoform c preproprotein) c4(247263) c5(247264, 247265, 247266, 247267, 247268, 247269, 247270) c6( ) c7(ID, tJ, T, uk, bM, Fv, xo, ago, e, lr, bO, fd, gf, HH, aR, ku, il, fm, bW, pX, Fo, Qk, M, iX, aU, abj, K, qM, aqD, oi, H, xa, aqn, KC, ZH, amy, LC, aqB, aW, wF, fc, abn, vt, Ku, Eg, as, eE, cm, mZ, yX, aqr, bb, eM, aqz, nB, ZT, U, vy, sv, nw, uJ, m, act, Zo, cZ, FW, gU, OI, My, fN, aqy, aj, ww, ct, nA, aqL, Vs, ke, Mo, ft, Hx, alq, aqJ, rl, Lv, lG, By, aqK, h, eV, dd, mV, mY, he, dx, aqi, mU, abf, aqg, PT, CN, A, aqA, dw, cM, fq, aqG, Jh, cv, mX, bJ, hq, ok, amt, aqp, cA, bP, dB, Ei, Be, hL, vl, at, JM, aqm, eo, lB, cB, mT, WI, B, L, bL, qc, it, jd, FM, iH, fG, lH, He, qQ, eX, aA, cR, mS, ti, cz, j, v, bX, aql, il, GC, V, q, J, aqs, aqF, er, eA, PW, aqt, di, aqN, yw, HJ, nv, WY, hb, uT, rS, aql, HC, aT, kf, cy, bK, an, rE, ae, apN, lr, aqC, af, eu, ak, aqv, if, jf, Y, yL, abi, ahe, r, cu, aaE, HL, en, cl, t, f, ik, FT, tl, ck, Qi, pH, cT, bN, ac, c, rR, yW, wD, mG, agH, Da, JH, EY, fD, gT, hl, kh, qX, VR, id, wi, aqh, iW, ha, jN, Gd, bS, aqu, fu, uu, qi, hF, ad, BY, dP, HF, aqq, hd, ot, mW, sO, aqk, aqx, xp, ahK, nJ, na, aqE, d, VX, aqj, tb, aaI, aog, et, Gv, ff, gC, zR, eZ, gl, aF, Z, FX, jb, bz, dr, DF, ek, Hw, EW, aqM, z, dJ, aaG, MJ, cP, ei, pk, lC, aqw, xg, iu, y); #c1 (BDP1) c2(NP_060899) c3 (transcription factor TFIIIB component B″ homolog) c4(198491) c5(198492, 198493, 198494, 198495, 198496, 198497, 198498, 198499, 198500, 198501, 198502, 198503, 198504, 198505, 198506, 198507, 198508, 198509) c6( ) c7(aW, T, ff, en, aT); #c1(BDP1) c2(XP_006714722) c3(transcription factor TFIIIB component B″ homolog isoform X3) c4( ) c5(288049, 288050, 288051, 288052, 288053, 288054) c6( ) c7(aW, T, ff, en, al); #c1(BDP1) c2(XP_006725523) c3(transcription factor TFIIIB component B″ homolog isoform X1) c4(296824) c5(296825, 296826, 296827, 296828, 296829, 296830, 296831, 296832, 296833) c6( ) c7(aW, T, ff, en, aT); #c1(BDP1) c2(XP_006725524) c3(transcription factor TFIIIB component B″ homolog isoform X3) c4(296834) c5( ) c6( ) c7(aW, T, ff, en, aT); #c1(BDP1) c2(XP_006726279) c3(transcription factor TFIIIB component B″ homolog isoform X1) c4(297140) c5(297141) c6( ) c7(aW, T, ff, en, aT); #c1(BDP1) c2(XP_011541815) c3(transcription factor TFIIIB component B″ homolog isoform X5) c4(340391) c5( ) c6( ) c7(aW, T, ff, en, aT); #c1(BEAN1) c2(NP_001184154) c3(protein BEAN1 isoform 3) c4(75430) c5(75431, 75432, 75433, 75434, 75435, 75436) c6( ) c7(bL, ky, kB, U, yZ, aqP, aqO, aqQ, dR); #c1(BEAN1) c2(XP_011521186) c3(protein BEAN1 isoform X1) c4(316116, 316117) c5(316118, 316119, 316120, 316121, 316122, 316123, 316124, 316125, 316126, 316127, 316128, 316129) c6( ) c7(bL, ky, kB, U, yZ, aqP, aqO, aql, dR); #c1(BEGAIN) c2(NP_001153003) c3(brain-enriched guanylate kinase-associated protein) c4( ) c5(60299, 60300, 60301, 60302, 60303, 60304, 60305, 60306, 60307, 60308, 60309, 60310, 60311, 60312, 60313, 60314, 60315, 60316) c6( ) c7(v); #c1(BEGAIN) c2(XP_005267976) c3(brain-enriched guanylate kinase-associated protein isoform X4) c4(281406, 281407) c5(281408, 281409, 281410) c6( ) c7(v); #c1(BEGAIN) c2(XP_005267977) c3(brain-enriched guanylate kinase-associated protein isoform X5) c4(281411) c5(281412) c6( ) c7(v); #c1(BEND3) c2(NP_001073919) c3(BEN domain-containing protein 3) c4(36531) c5(36532, 36533, 36534, 36535, 36536, 36537, 36538, 36539, 36540, 36541) c6( ) c7(aqR, H, Cb, L); #c1(BEND4) c2(NP_001153019) c3(BEN domain-containing protein 4 isoform b) c4(60329) c5(60330, 60331, 60332, 60333, 60334) c6( ) c7(r); #c1(BEND4) c2(NP_997289) c3(BEN domain-containing protein 4 isoform a) c4(265375, 265376) c5(265377, 265378, 265379, 265380, 265381, 265382, 265383, 265384, 265385, 265386, 265387, 265388, 265389) c6( ) c7(r); #c1(BEST1) c2(NP_001132915) c3(bestrophin-1 isoform 2) c4( ) c5(52646, 52647, 52648, 52649, 52650) c6( ) c7(bL, bM, T, dD, ef, arb, Ol, nx, ea, arh, Ri, cz, n, aj, jA, eq, aqS, sl, afl, cd, J, Nh, cc, ard, aE, aqT, vJ, il, Sr, ari, oj, adX, kf, Ak, Ps, arc, bl, aqX, zp, afV, sa, bA, ara, lR, aqZ, kj, are, xz, ahk, xs, Hz, lb, uY, hp, JI, nC, dr, qa, eY, Bv, W, nb, adZ, aqU, yE, aqW, mx, U, km, aqV, rx, arf, mf, m, ei, ol, y, arg, aqY, ao); #c1(BEST1) c2(NP_001287715) c3(bestrophin-1 isoform 3) c4(112623) c5(112624, 112625, 112626, 112627, 112628) c6( ) c7(bL, bM, T, dD, ef, arb, Ol, nx, ea, arh, Ri, cz, n, aj, jA, eq, aqS, sl, afl, cd, J, Nh, cc, ard, aE, aqT, vJ, il, Sr, ari, oj, adX, kf, Ak, Ps, arc, bl, aqX, zp, afV, sa, bA, ara, lR, aqZ, kj, are, xz, ahk, xs, Hz, lb, uY, hp, JI, nC, dr, qa, eY, Bv, W, nb, adZ, aqU, yE, aqW, mx, U, km, aqV, rx, arf, mf, m, ei, ol, y, arg, aqY, ao); #c1(BEST1) c2(NP_004174) c3(bestrophin-1 isoform 1) c4(144092) c5(144093, 144094, 144095, 144096, 144097, 144098, 144099) c6( ) c7(bL, bM, T, dD, ef, arb, Ol, nx, ea, arh, Ri, cz, n, aj, jA, eq, aqS, sl, afl, cd, J, Nh, cc, ard, aE, aqT, vJ, il, Sr, ari, oj, adX, kf, Ak, Ps, arc, bl, aqX, zp, afV, sa, bA, ara, lR, aqZ, kj, are, xz, ahk, xs, Hz, lb, uY, hp, Jl, nC, dr, qa, eY, Bv, W, nb, adZ, aqu, yE, aqW, mx, U, km, aqV, rx, arf, mf, m, ei, oQ, y, arg, aqY, ao); #c1(BEST1) c2(XP_005274275) c3(bestrophin-1 isoform X8) c4( ) c5(285032) c6( ) c7(bL, bM, T, dD, ef, arb, Ol, nx, ea, arh, Ri, cz, n, aj, jA, eq, aqS, sl, afl, cd, J, Nh, cc, ard, aE, aqT, vJ, il, Sr, ari, oj, adX, kf, Ak, Ps, arc, bl, aqX, zp, afV, sa, bA, ara, lR, aqZ, kj, are, xz, ahk, xs, Hz, lb, uY, hp, JI, nC, dr, qa, eY, Bv, W, nb, adZ, aqu, yE, aqW, mx, U, km, aqV, rx, arf, mf, m, ei, ol, y, arg, aqY, ao); #c1(BEST1) c2(XP_005274278) c3(bestrophin-1 isoform X10) c4(285033) c5(285034, 285035, 285036, 285037, 285038, 285039) c6( ) c7(bL, bM, T, dD, ef, arb, Ol, nx, ea, arh, Ri, cz, n, aj, jA, eq, aqS, sl, afl, cd, J, Nh, cc, ard, aE, aqT, vJ, il, Sr, ari, oj, adX, kf, Ak, Ps, arc, bl, aqX, zp, afV, sa, bA, ara, lR, aqZ, kj, are, xz, ahk, xs, Hz, lb, uY, hp, JI, nC, dr, qa, eY, Bv, W, nb, adZ, aqu, yE, aqW, mx, U, km, aqV, rx, arf, mf, m, ei, ol, y, arg, aqY, ao); #c1(BEST1) c2(XP_011543531) c3(bestrophin-1 isoform X1) c4(342448, 342449) c5( ) c6( ) c7(bL, bM, T, dD, ef, arb, Ol, nx, ea, arh, Ri, cz, n, aj, jA, eq, aqS, sl, afl, cd, J, Nh, cc, ard, aE, aqT, vJ, il, Sr, ari, oj, adX, kf, Ak, Ps, arc, bl, aqX, zp, afV, sa, bA, ara, lR, aqZ, kj, are, xz, ahk, xs, Hz, lb, uY, hp, Jl, nC, dr, qa, eY, Bv, W, nb, adZ, aqu, yE, aqW, mx, U, km, aqV, rx, arf, mf, m, ei, od, y, arg, aqY, ao); #c1(BEST1) c2(XP_011543532) c3(bestrophin-1 isoform X2) c4( ) c5(342450) c6( ) c7(bL, bM, T, dD, ef, arb, Ol, nx, ea, arh, Ri, cz, n, aj, jA, eq, aqS, sl, afl, cd, J, Nh, cc, ard, aE, aqT, vJ, il, Sr, ari, oj, adX, kf, Ak, Ps, arc, bl, aqX, zp, afV, sa, bA, ara, lR, aqZ, kj, are, xz, ahk, xs, Hz, lb, uY, hp, JI, nC, dr, qa, eY, Bv, W, nb, adZ, aqu, yE, aqW, mx, U, km, aqV, rx, arf, mf, m, ei, ol, y, arg, aqY, ao); #c1 (BEST1) c2(XP_011543535) c3(bestrophin-1 isoform X6) c4( ) c5(342451, 342452, 342453, 342454, 342455, 342456) c6( ) c7(bL, bM, T, dD, ef, arb, Ol, nx, ea, arh, Ri, cz, n, aj, jA, eq, aqS, sl, afl, cd, J, Nh, cc, ard, aE, aqT, vJ, il, Sr, ari, oj, adX, kf, Ak, Ps, arc, bl, aqX, zp, afV, sa, bA, ara, lR, aqZ, kj, are, xz, ahk, xs, Hz, lb, uY, hp, JI, nC, dr, qa, eY, Bv, W, nb, adZ, aqu, yE, aqW, mx, U, km, aqV, rx, arf, mf, m, ei, ol, y, arg, aqY, ao); #c1(BEST2) c2(XP_005260020) c3(bestrophin-2 isoform X1) c4(276385) c5(276386, 276387, 276388, 276389, 276390, 276391, 276392, 276393, 276394, 276395, 276396, 276397, 276398, 276399, 276400, 276401) c6( ) c7(IV, DD); #c1(BET1) c2(NP_005859) c3(BET1 homolog) c4(159990) c5(159991, 159992, 159993, 159994, 159995, 159996) c6( ) c7(vA); #c1(BET1L) c2(NP_001092257) c3(BET1-like protein isoform 1) c4(39474) c5(39475, 39476, 39477, 39478, 39479, 39480) c6( ) c7(op, vA, us, afW); #c1(BET1L) c2(NP_057610) c3 (BET1-like protein isoform 2) c4(191747) c5(191748, 191749, 191750, 191751, 191752, 191753) c6( ) c7(op, vA, us, afW); #c1(BEX1) c2(NP_060946) c3(protein BEX1) c4(198854) c5(198855, 198856, 198857, 198858, 198859) c6( ) c7(ck, iC, T, d, aW, ff, aT, en, Fc, eA); #c1(BEX2) c2(NP_001161871) c3(protein BEX2 isoform 1) c4(66250) c5(66251, 66252, 66253, 66254, 66255, 66256) c6( ) c7(ck, iC, T, d, aW, ff, aT, en, Fc, eA); #c1(BEX2) c2(NP_001161872) c3(protein BEX2 isoform 2) c4(66257) c5(66258, 66259, 66260, 66261) c6( ) c7(ck, iC, T, d, aW, ff, aT, en, Fc, eA); #c1(BEX2) c2(NP_001161873) c3(protein BEX2 isoform 3) c4(66262) c5(66263, 66264, 66265, 66266, 66267, 66268, 66269, 66270, 66271, 66272, 66273, 66274, 66275) c6( ) c7(ck, iC, T, d, aW, ff, aT, en, Fc, eA); #c1(BEX4) c2(NP_001073894) c3(protein BEX4) c4(36344) c5(36345, 36346, 36347, 36348, 36349, 36350, 36351, 36352, 36353, 36354) c6( ) c7(t, m); #c1(BFAR) c2(NP_057645) c3(bifunctional apoptosis regulator) c4(192019) c5(192020, 192021, 192022, 192023, 192024, 192025, 192026) c6( ) c7(bL, T, pf, JD, arj, U, cj, uS, dq, agD, v, eo, K, qM, GF); #c1(BFAR) c2(XP_005255407) c3(bifunctional apoptosis regulator isoform X2) c4(273631) c5(273632, 273633, 273634, 273635, 273636, 273637) c6( ) c7(bL, T, pf, JD, arj, U, cj, uS, dq, agD, v, eo, K, qM, GF); #c1(BFSP1) c2(NP_001155/77) c3(filensin isoform 2) c4(61492) c5(61493, 61494, 61495, 61496, 61497) c6( ) c7(arl, dY, ark); #c1(BFSP1) c2(NP_001265536) c3(filensin isoform 4) c4( ) c5(98761, 98762) c6( ) c7(arl, dY, ark); #c1(BFSP1) c2(NP_001265537) c3(filensin isoform 3) c4(98763) c5(98764, 98765, 98766, 98767, 98768, 98769, 98770, 98771) c6( ) c7(arl, dY, ark); #c1(BFSP2) c2(NP_003562) c3(phakinin) c4(139195) c5(139196, 139197, 139198, 139199, 139200, 139201) c6( ) c7(kj, arm, arn, aro, Zh, Hr); #c1(BGLAP) c2(NP_954642) c3(osteocalcin preproprotein) c4(261148) c5(261149, 261150, 261151, 261152, 261153, 261154) c6( ) c7(gh, T, bM, AB, dA, aC, id, eR, zs, zt, Ay, cz, bB, aj, dG, j, ci, v, fr, fd, aY, amC, eW, J, ad, afb, amB, zw, RN, rC, ari, aT, afV, zp, bq, rv, ae, ars, af, bA, hi, A, s, up, cW, aW, Hz, eH, arp, arq, eF, arv, arr, acW, nM, yE, TJ, bE, wB, aru, wR, mf, k, lS, art, bi, y, LS); #c1(BGN) c2(NP_001702) c3(biglycan preproprotein) c4(120216, 120217, 120218) c5(120219, 120220, 120221, 120222, 120223, 120224, 120225, 120226, 120227, 120228, 120229, 120230, 120231, 120232, 120233, 120234, 120235, 120236, 120237, 120238, 120239, 120240) c6( ) c7(ID, rt, T, dD, aC, pG, id, ea, fG, e, aw, aA, lj, arw, v, eq, kv, uW, mC, q, hd, aE, hW, vJ, db, dv, lC, aT, Ak, cy, dl, ey, K, zp, H, vO, eL, VC, ly, eG, bf, anP, LC, aW, eH, eZ, r, aq, aF, si, zC, pL, eF, wU, qa, lx, bF, GU, bE, bm, cj, ro, aS, arx, fL, R, dy, fy, B, y, vQ); #c1(BHLHA15) c2(NP_803238) c3(class A basic helix-loop-helix protein 15) c4(252586) c5(252587, 252588, 252589, 252590, 252591, 252592) c6( ) c7(es, pe, aA, fd, kp, aF, ci, ary, aom, fr, VB); #c1(BHLHA9) c2(NP_001157877) c3(class A basic helix-loop-helix protein 9) c4(62850) c5(62851, 62852, 62853, 62854, 62855, 62856, 62857) c6( ) c7(arz, arB, arA); #c1(BHLHB9) c2(NP_001135999) c3(protein BHLHb9) c4(53943) c5(53944, 53945, 53946, 53947, 53948, 53949, 53950, 53951, 53952, 53953, 53954, 53955, 53956, 53957, 53958, 53959, 53960, 53961, 53962, 53963, 53964, 53965) c6( ) c7(af, U); #c1(BHLHE22) c2(NP_689627) c3(class E basic helix-loop-helix protein 22) c4(243331) c5(243332, 243333, 243334, 243335, 243336, 243337, 243338) c6( ) c7(T, ea, if, al, fd, ci, Na, bv, ta, DY, eL, aW, nY, g, as, pl, eF, cm, DL, eM, mx, aiM, m, cZ, dt, dD, vl, aw, aj, fr, qN, ai, rc, zw, h, By, dL, dd, A, arG, arf, dB, vb, agl, XN, B, bL, aA, arD, zN, cz, v, j, ajM, Eh, q, J, eA, arH, aT, ae, arl, PK, HZ, Y, r, lo, t, arJ, ck, acW, AH, Uq, rA, kh, uu, eq, ad, dP, Dq, ji, lQ, Jg, d, eH, aF, qa, gk, arE, arC, HN, ll, gZ, dX, XO, ei, y); #c1(BHLHE23) c2(NP_542173) c3(class E basic helix-loop-helix protein 23) c4(232176) c5(232177, 232178, 232179, 232180, 232181, 232182, 232183) c6( ) c7(sc, T, arM, dt, aC, Dh, e, ha, jN, l, XJ, aA, n, bB, fN, fd, ci, jh, arN, Eh, XM, q, aR, br, ad, Fc, ft, aE, lg, aT, h, dL, K, lQ, akr, H, arL, ae, eL, HO, arO, hM, Jg, XK, d, eG, A, aW, ev, Ls, xs, Gv, ff, r, lo, ark, aF, en, g, cm, XI, bz, pb, dr, bw, es, sS, eM, bE, X, HN, wB, PV, XG, XO, XN, it, B, XL, L); #c1(BHLHE40) c2(NP_003661) c3(class E basic helix-loop-helix protein 40) c4(140034) c5(140035, 140036, 140037, 140038, 140039, 140040) c6( ) c7(T, aC, ht, aA, ajM, j, ci, fr, py, aE, Ae, aT, jy, bK, K, hu, bA, aW, Y, ff, eZ, aq, aF, en, Z, iK, bz, NC, eM, bE, it, B); #c1(BHLHE41) c2(NP_110389) c3(class E basic helix-loop-helix protein 41) c4(219857, 219858) c5(219859, 219860, 219861, 219862, 219863, 219864, 219865, 219866, 219867, 219868, 219869, 219870, 219871, 219872, 219873, 219874, 219875, 219876, 219877, 219878, 219879, 219880, 219881, 219882, 219883, 219884, 219885, 219886, 219887, 219888, 219889, 219890, 219891, 219892, 219893, 219894, 219895, 219896, 219897, 219898, 219899, 219900, 219901, 219902, 219903, 219904, 219905, 219906, 219907, 219908, 219909, 219910, 219911, 219912, 219913, 219914, 219915, 219916, 219917, 219918, 219919, 219920, 219921, 219922, 219923, 219924, 219925, 219926, 219927, 219928, 219929, 219930, 219931, 219932, 219933, 219934, 219935, 219936, 219937, 219938, 219939, 219940, 219941, 219942, 219943, 219944, 219945, 219946, 219947, 219948, 219949, 219950, 219951, 219952, 219953, 219954, 219955, 219956, 219957, 219958, 219959) c6( ) c7(av, T, kP, aW, arP, aT, kp, np, ci, bK, g, Z, bz, iu, pl); #c1(BHMT) c2(NP_001704) c3(betaine-homocysteine S-methyltransferase 1) c4(120255, 120256) c5(120257, 120258, 120259, 120260, 120261, 120262, 120263, 120264, 120265, 120266, 120267, 120268) c6( ) c7(bL, dD, iW, e, aT, ard, sM, v, j, eq, md, av, Na, amJ, aT, Nc, dL, eu, wC, aW, fc, r, LI, Lk, f, ac, eM, lp, pE, wE, bm, at, aS, cP, bD, B); #c1(BHMT2) c2(NP_001171476) c3(S-methylmethionine-homocysteine S-methyltransferase BHMT2 isoform 2) c4(70264) c5(70265, 70266, 70267, 70268, 70269, 70270) c6( ) c7(Na, dD, ac, lp, wE, bm, Nc, eq, j, bD, eu); #c1(BHMT2) c2(NP_060084) c3(S-methylmethionine-homocysteine S-methyltransferase BHMT2 isoform 1) c4(194078) c5(194079, 194080, 194081, 194082, 194083, 194084) c6( ) c7(Na, dD, ac, lp, wE, bm, Nc, eq, j, bD, eu); #c1(BICC1) c2(NP_001073981) c3(protein bicaudal C homolog 1) c4(36769) c5(36770, 36771, 36772, 36773, 36774, 36775) c6( ) c7(MS, arT, arS, arV, wB, arR, arU, bO, aj, bS, dy, ct, Nj, Z, J); #c1 (BICC1) c2(XP_005270226) c3(protein bicaudal C homolog 1 isoform X4) c4(283290) c5( ) c6( ) c7(MS, arT, arS, arV, wB, arR, arl, bO, aj, bS, dy, ct, Nj, Z, J); #c1 (BICC1) c2(XP_011538493) c3(protein bicaudal C homolog 1 isoform X8) c4( ) c5(336242, 336243, 336244, 336245, 336246, 336247, 336248) c6( ) c7(MS, arT, arS, arV, wB, arR, arU, bO, aj, bS, dy, ct, Nj, Z, J); #c1(BICD1) c2(NP_001003398) c3(protein bicaudal D homolog 1 isoform 2) c4(19816) c5(19817, 19818, 19819, 19820, 19821, 19822) c6( ) c7(aC, at, y, V, sD, ad); #c1(BICD1) c2(NP_001705) c3(protein bicaudal D homolog 1 isoform 1) c4(120269) c5(120270, 120271, 120272, 120273, 120274, 120275) c6( ) c7(aC, at, y, V, sD, ad); #c1 (BICD1) c2(XP_005253522) c3(protein bicaudal D homolog 1 isoform X6) c4(272544) c5(272545, 272546, 272547, 272548, 272549) c6( ) c7(aC, at, y, V, sD, ad); #c1(BICD1) c2(XP_011519116) c3(protein bicaudal D homolog 1 isoform X3) c4( ) c5(313724) c6( ) c7(aC, at, y, V, sD, ad); #c1(BICD2) c2(NP_001003800) c3(protein bicaudal D homolog 2 isoform 1) c4(20148) c5(20149, 20150, 20151, 20152, 20153, 20154) c6( ) c7(kf, arW, aV); #c1(BICD2) c2(NP_056065) c3(protein bicaudal D homolog 2 isoform 2) c4(185488) c5(185489, 185490, 185491, 185492, 185493, 185494) c6( ) c7(kf, arW, aV); #c1(BID) c2(NP_001187) c3(BH3-interacting domain death agonist isoform 2) c4(78964, 78965) c5(78966, 78967, 78968, 78969, 78970, 78971, 78972, 78973, 78974, 78975, 78976, 78977, 78978, 78979, 78980) c6( ) c7(T, dD, fM, sw, ea, if, jx, iW, aA, j, ci, eq, qN, ad, XW, av, Bc, aT, h, lG, bK, K, fb, ae, af, dx, A, B, iU, aW, Y, r, lo, aF, g, bz, cj, bt, X); #c1(BID) c2(NP_001231499) c3(BH3-interacting domain death agonist isoform 3) c4( ) c5(87259, 87260, 87261, 87262, 87263, 87264, 87265, 87266) c6( ) c7(T, dD, fM, sw, ea, if, jx, iW, aA, j, ci, eq, qN, ad, XW, av, Bc, aT, h, lG, bK, K, fb, ae, af, dx, A, B, iU, aW, Y, r, lo, aF, g, bz, cj, bt, X); #c1(BID) c2(NP_932070) c3(BH3-interacting domain death agonist isoform 1) c4(258793, 258794, 258795, 258796) c5(258797, 258798, 258799, 258800, 258801, 258802, 258803, 258804, 258805) c6( ) c7(T, dD, fM, sw, ea, if, jx, iW, aA, j, ci, eq, qN, ad, XW, av, Bc, aT, h, lG, bK, K, fb, ae, af, dx, A, B, iU, aW, Y, r, lo, aF, g, bz, cj, bt, X); #c1(BIN1) c2(NP_004296) c3(myc box-dependent-interacting protein 1 isoform 8) c4(144952) c5(144953, 144954, 144955, 144956, 144957, 144958) c6( ) c7(T, UU, Or, bB, ci, UX, cd, fK, ad, cc, oj, aT, bU, ae, af, aW, Y, xz, aq, cm, dR, ac, bE, X, B, UV); #c1(BINI) c2(NP_647593) c3(myc box-dependent-interacting protein 1 isoform 1) c4(238734) c5(238735, 238736, 238737, 238738, 238739, 238740) c6( ) c7(T, UU, Or, bB, ci, UX, cd, fK, ad, cc, oj, aT, bU, ae, af, aW, Y, xz, aq, cm, dR, ac, bE, X, B, UV); #c1(BINI) c2(NP_647594) c3(myc box-dependent-interacting protein 1 isoform 2) c4(238741) c5(238742, 238743, 238744, 238745, 238746, 238747) c6( ) c7(T, UU, Or, bB, ci, UX, cd, fK, ad, cc, oj, aT, bU, ae, af, aW, Y, xz, aq, cm, dR, ac, bE, X, B, UV); #c1(BINI) c2(NP_647595) c3(myc box-dependent-interacting protein 1 isoform 3) c4(238748) c5(238749, 238750, 238751, 238752, 238753, 238754) c6( ) c7(T, UU, Or, bB, ci, UX, cd, fK, ad, cc, oj, aT, bU, ae, af, aW, Y, xz, aq, cm, dR, ac, bE, X, B, UV); #c1(BIN1) c2(NP_647596) c3(myc box-dependent-interacting protein 1 isoform 4) c4(238755) c5(238756, 238757, 238758, 238759, 238760, 238761) c6( ) c7(T, UU, Or, bB, ci, UX, cd, fK, ad, cc, oj, aT, bU, ae, af, aW, Y, xz, aq, cm, dR, ac, bE, X, B, UV); #c1(BIN1) c2(NP_647597) c3(myc box-dependent-interacting protein 1 isoform 5) c4(238762) c5(238763, 238764, 238765, 238766, 238767, 238768) c6( ) c7(T, UU, Or, bB, ci, UX, cd, fk, ad, cc, oj, aT, bu, ae, af, aW, Y, xz, aq, cm, dR, ac, bE, X, B, UV); #c1(BIN1) c2(NP_647598) c3(myc box-dependent-interacting protein 1 isoform 6) c4(238769) c5(238770, 238771, 238772, 238773, 238774, 238775) c6( ) c7(T, UU, Or, bB, ci, UX, cd, fK, ad, cc, oj, aT, bU, ae, af, aW, Y, xz, aq, cm, dR, ac, bE, X, B, UV); #c1(BINI) c2(NP_647599) c3(myc box-dependent-interacting protein 1 isoform 7) c4(238776) c5(238777, 238778, 238779, 238780, 238781, 238782) c6( ) c7(T, UU, Or, bB, ci, UX, cd, fK, ad, cc, oj, aT, bU, ae, af, aW, Y, xz, aq, cm, dR, ac, bE, X, B, UV); #c1(BINI) c2(NP_647600) c3(myc box-dependent-interacting protein 1 isoform 9) c4(238783) c5(238784, 238785, 238786, 238787, 238788, 238789) c6( ) c7(T, UU, Or, bB, ci, UX, cd, fK, ad, cc, oj, aT, bU, ae, af, aW, Y, xz, aq, cm, dR, ac, bE, X, B, UV); #c1(BINI) c2(NP_647601) c3(myc box-dependent-interacting protein 1 isoform 10) c4(238790) c5(238791, 238792, 238793, 238794, 238795, 238796) c6( ) c7(T, UU, Or, bB, ci, UX, cd, fK, ad, cc, oj, aT, bU, ae, af, aW, Y, xz, aq, cm, dR, ac, bE, X, B, UV); #c1(BIN2) c2(NP_001276936) c3(bridging integrator 2 isoform 2) c4( ) c5(108683, 108684, 108685, 108686, 108687, 108688) c6( ) c7(T); #c1(BIN2) c2(NP_001276937) c3(bridging integrator 2 isoform 3) c4(108689) c5(108690, 108691, 108692, 108693, 108694, 108695) c6( ) c7(T); #c1(BIN2) c2(NP_057377) c3(bridging integrator 2 isoform 1) c4(190469, 190470) c5(190471, 190472, 190473, 190474, 190475, 190476) c6( ) c7(T); #c1(BIN3) c2(NP_061158) c3(bridging integrator 3) c4(199645, 199646) c5(199647, 199648, 199649, 199650, 199651, 199652) c6( ) c7(cd); #c1(BIN3) c2(XP_005273630) c3(bridging integrator 3 isoform X1) c4( ) c5(284721) c6( ) c7(cd); #c1(BIN3) c2(XP_011542888) c3(bridging integrator 3 isoform X2) c4( ) c5(341484, 341485, 341486, 341487, 341488) c6( ) c7(cd); #c1(BIRC2) c2(NP_001243092) c3(baculoviral IAP repeat-containing protein 2 isoform 1) c4(89750, 89751, 89752) c5(89753, 89754, 89755, 89756, 89757, 89758, 89759, 89760, 89761, 89762, 89763, 89764, 89765, 89766, 89767, 89768, 89769, 89770, 89771, 89772, 89773, 89774, 89775, 89776, 89777, 89778, 89779, 89780, 89781, 89782, 89783, 89784, 89785, 89786, 89787, 89788, 89789, 89790, 89791, 89792, 89793, 89794, 89795, 89796, 89797, 89798, 89799, 89800, 89801, 89802, 89803, 89804, 89805, 89806, 89807, 89808, 89809, 89810, 89811, 89812, 89813, 89814, 89815, 89816, 89817, 89818, 89819, 89820, 89821, 89822, 89823, 89824, 89825, 89826, 89827, 89828, 89829, 89830, 89831, 89832, 89833, 89834, 89835, 89836, 89837, 89838, 89839, 89840, 89841, 89842, 89843, 89844, 89845, 89846, 89847, 89848, 89849, 89850, 89851, 89852, 89853, 89854, 89855, 89856, 89857, 89858, 89859, 89860, 89861) c6( ) c7(gh, T, fM, qb, if, aA, fd, n, fr, ci, qN, V, arX, q, ad, afb, x, av, oj, kH, aT, h, dL, K, ae, je, apl, nO, aW, Y, ff, xs, eH, uY, en, eF, t, Bu, bz, ik, bw, ck, arY, iM, bE, hL, cj, k, bC, bt, it, B, cn); #c1(BIRC2) c2(NP_001243095) c3(baculoviral IAP repeat-containing protein 2 isoform 2) c4(89862) c5(89863, 89864, 89865, 89866, 89867, 89868) c6( ) c7(gh, T, fM, qb, if, aA, fd, n, fr, ci, qN, V, arX, q, ad, afb, x, av, oj, kH, aT, h, dL, K, ae, je, apl, nO, aW, Y, ff, xs, eH, uY, en, eF, t, Bu, bz, ik, bw, ck, arY, iM, bE, hL, cj, k, bC, bt, it, B, cn); #c1(BIRC3) c2(NP_892007) c3(baculoviral IAP repeat-containing protein 3) c4(257471) c5(257472, 257473, 257474, 257475, 257476, 257477, 257478, 257479, 257480, 257481, 257482, 257483, 257484, 257485, 257486, 257487, 257488, 257489, 257490, 257491, 257492, 257493, 257494, 257495, 257496, 257497, 257498, 257499, 257500, 257501, 257502, 257503, 257504, 257505, 257506, 257507, 257508, 257509, 257510, 257511, 257512, 257513, 257514, 257515, 257516, 257517, 257518, 257519, 257520, 257521, 257522, 257523, 257524, 257525, 257526, 257527, 257528, 257529, 257530, 257531, 257532, 257533, 257534, 257535, 257536, 257537, 257538, 257539, 257540, 257541, 257542, 257543, 257544, 257545, 257546, 257547, 257548, 257549, 257550, 257551, 257552, 257553, 257554, 257555, 257556, 257557, 257558, 257559, 257560, 257561, 257562, 257563, 257564, 257565, 257566, 257567, 257568, 257569) c6( ) c7(gh, T, fM, fR, qb, ea, if, df, iW, jh, asa, bB, fd, fr, ci, qN, V, asb, q, ad, afb, av, ft, ib, aE, ds, kH, aT, h, dL, NM, ae, aa, akV, d, B, iU, nO, aW, Y, oV, az, eH, fc, ff, aq, lo, aF, en, g, eF, t, iV, bz, bw, arZ, ck, asc, eM, bE, cj, at, SZ, m, bC, X); #c1(BIRC5) c2(NP_001012270) c3 (baculoviral IAP repeat-containing protein 5 isoform 2) c4(24825) c5(24826, 24827, 24828, 24829, 24830, 24831) c6( ) c7(bM, T, aC, fR, if, fd, bB, ci, ip, x, aE, rw, hu, eL, aW, hl, apm, g, as, cm, bw, eM, nB, AM, nF, iA, iT, cj, k, m, iu, sw, aw, ht, n, fr, qN, cd, Bl, ft, jz, hw, cc, kH, h, lG, dl, wb, ey, hB, EU, AQ, fQ, aq, cM, bP, dB, apl, hL, it, B, fM, jp, aA, hy, q, eA, Vr, aT, rO, Tj, ae, ne, jf, s, iU, Y, r, en, cl, t, ck, nM, kP, iM, im, rA, bt, GI, kh, id, LX, iW, jh, Fc, ase, ad, asd, hX, dP, av, hd, ib, NS, io, iY, hP, d, BN, lU, je, qt, ff, Ej, aF, gW, iR, bz, bF, es, arY, Ew, bE, Lb, lC, X, y); #c1(BIRC5) c2(NP_001012271) c3(baculoviral IAP repeat-containing protein 5 isoform 3) c4(24832, 24833) c5(24834, 24835, 24836, 24837, 24838, 24839, 24840, 24841, 24842, 24843, 24844, 24845) c6( ) c7(bM, T, aC, fR, if, fd, bB, ci, ip, x, aE, rw, hu, eL, aW, hl, apm, g, as, cm, bw, eM, nB, AM, nF, iA, iT, cj, k, m, iu, sw, aw, ht, n, fr, qN, cd, Bl, ft, jz, hw, cc, kH, h, lG, dL, wb, ey, hB, EU, AQ, fQ, aq, cM, bP, dB, apl, hL, it, B, fM, jp, aA, hy, q, eA, Vr, aT, rO, Tj, ae, ne, jf, s, iU, Y, r, en, cl, t, ck, nM, kP, iM, im, rA, bt, GI, kh, id, LX, iW, jh, Fc, ase, ad, asd, hX, dP, av, hd, ib, NS, io, iY, hP, d, BN, lU, je, qt, ff, Ej, aF, gW, iR, bz, bF, es, arY, Ew, bE, Lb, lC, X, y); #c1(BIRC5) c2(NP_001159) c3(baculoviral IAP repeat-containing protein 5 isoform 1) c4(65021) c5( ) c6( ) c7(bM, T, aC, fR, if, fd, bB, ci, ip, x, aE, rw, hu, eL, aW, hl, apm, g, as, cm, bw, eM, nB, AM, nF, iA, iT, cj, k, m, iu, sw, aw, ht, n, fr, qN, cd, Bl, ft, jz, hw, cc, kH, h, lG, dl, wb, ey, hB, EU, AQ, fQ, aq, cM, bP, dB, apl, hL, it, B, fM, jp, aA, hy, q, eA, Vr, aT, rO, Tj, ae, ne, jf, s, iU, Y, r, en, cl, t, ck, nM, kP, iM, im, rA, bt, Gl, kh, id, LX, iW, jh, Fc, ase, ad, asd, hX, dP, av, hd, ib, NS, io, iY, hP, d, BN, lU, je, qt, ff, Ej, aF, gW, iR, bz, bF, es, arY, Ew, bE, Lb, lC, X, y); #c1(BIRC6) c2(NP_057336) c3(baculoviral IAP repeat-containing protein 6) c4(190178) c5(190179, 190180, 190181, 190182, 190183, 190184) c6( ) c7(T, BN, eM, dH, bE, aW, Y, Dd, Qu, aT, bB, hy, cm, ae, ne, ad); #c1 (BIRC7) c2(NP_071444) c3(baculoviral IAP repeat-containing protein 7 isoform beta) c4(211702, 211703) c5(211704, 211705, 211706, 211707, 211708, 211709, 21/710, 211711, 211712, 211713, 211714, 211715) c6( ) c7(T, iW, bB, n, fr, ci, hF, av, Vr, aT, dL, cp, DA, asf, nO, Y, as, eF, cm, t, bz, eM, bE, Lb, m, k); #c1(BIRC7) c2(NP_647478) c3(baculoviral IAP repeat-containing protein 7 isoform alpha) c4(238663) c5(238664, 238665, 238666, 238667, 238668, 238669) c6( ) c7(T, iW, bB, n, fr, ci, hF, av, Vr, aT, dL, cp, DA, asf, nO, Y, as, eF, cm, t, bz, eM, bE, Lb, m, k); #c1(BIVM) c2(NP_001153068) c3(basic immunoglobulin-like variable motif-containing protein isoform b) c4(60402) c5(60403, 60404, 60405, 60406, 60407) c6( ) c7(cd, bk, ac); #c1(BIVM) c2(NP_060163) c3(basic immunoglobulin-like variable motif-containing protein isoform a) c4(194539) c5(194540, 194541, 194542, 194543, 194544, 194545, 194546, 194547, 194548, 194549, 194550, 194551, 194552) c6( ) c7(cd, bK, ac); #c1(BLCAP) c2(NP_001161292) c3(bladder cancer-associated protein) c4(65519, 65520, 65521) c5(65522, 65523, 65524, 65525, 65526, 65527, 65528, 65529, 65530, 65531, 65532, 65533, 65534, 65535, 65536, 65537, 65538, 65539, 65540, 65541, 65542, 65543, 65544, 65545, 65546, 65547, 65548, 65549, 65550, 65551, 65552, 65553, 65554, 65555, 65556, 65557, 65558, 65559, 65560, 65561, 65562, 65563) c6( ) c7(es, T, iA, aW, if, eH, aT, dL, ci, qN, eF, eL, er); #c1(BLID) c2(NP_001001786) c3(BH3-like motif-containing cell death inducer) c4(19020) c5(19021, 19022, 19023, 19024, 19025, 19026) c6( ) c7(x, T, asg, ac, aW, e, aT, k); #c1(BLK) c2(NP_001706) c3(tyrosine-protein kinase Blk) c4(120276, 120277) c5(120278, 120279, 120280, 120281, 120282, 120283, 120284, 120285, 120286, (20287, 120288, 120289) c6( ) c7(bL, fN, ez, aj, v, V, mC, J, Bc, bW, ey, OV, A, iU, eZ, pb, Bv, yW, ash, lf, cj, aS, fL, y, Pr); #c1(BLM) c2(NP_001274175) c3(Bloom syndrome protein isoform 1) c4(106109, 106110, 106111) c5(106112, 106113, 106114, 106115, 106116, 106117, 106118, 106119, 106120, 106121, 106122, 106123) c6( ) c7(T, os, fM, dD, eR, ea, iW, NT, ew, oZ, eq, ci, q, av, asi, ft, MR, aT, h, dL, K, jj, ask, eL, asj, cp, Qe, wr, BO, aW, Y, r, La, lk, dr, ck, Qi, gX, at, bC, bt, kS, sH); #c1(BLM) c2(XP_006720695) c3(Bloom syndrome protein isoform X3) c4( ) c5(293611, 293612, 293613, 293614, 293615, 293616) c6( ) c7(T, os, fM, dD, eR, ea, iW, NT, ew, oZ, eq, ci, q, av, asi, ft, MR, aT, h, dL, K, jj, ask, eL, asj, cp, Qe, wr, BO, aW, Y, r, La, lk, dr, ck, Qi, gX, at, bC, bt, KS, sH); #c1(BLMH) c2(NP_000377) c3(bleomycin hydrolase) c4(8288, 8289) c5(8290, 8291, 8292, 8293, 8294, 8295, 8296, 8297, 8298, 8299, 8300, 8301) c6( ) c7(af, aW, fG, Lf, U, aT, dN, Lb, asl, bu, eE, cM, B); #c1(BLNK) c2(NP_001107566) c3(B-cell linker protein isoform 2) c4(42865, 42866, 42867) c5(42868, 42869, 42870, 42871, 42872, 42873, 42874) c6( ) c7(oO, Kd, T, fM, BD, dA, ra, aD, jx, jh, fd, bB, C, uE, asr, V, mC, ai, x, F, hD, h, hn, ey, ji, pk, aso, hM, asn, asp, A, oV, asm, bg, Pg, eZ, Of, Og, CQ, Wt, ck, ba, ou, iQ, zr, bE, asq, lf, cj, uz, oX, k, rH, fL, hh, fD, Ze, y); #c1(BLNK) c2(NP_001245369) c3(B-cell linker protein isoform 3) c4(93312, 93313, 93314, 93315) c5(93316, 93317, 93318, 93319, 93320, 93321, 93322, 93323, 93324, 93325, 93326, 93327) c6( ) c7(oO, Kd, T, fM, BD, dA, ra, aD, jx, jh, fd, bB, C, uE, asr, V, mC, ai, x, F, hD, h, hn, ey, ji, pk, aso, hM, asn, asp, A, oV, asm, bg, Pg, eZ, Of, Og, CO, Wt, ck, ba, ou, iQ, zr, bE, asq, lf, cj, uz, oX, k, rH, FL, hh, fD, Ze, y); #c1(BLNK) c2(NP_001245370) c3(B-cell linker protein isoform 4) c4( ) c5(93328, 93329, 93330, 93331, 93332, 93333, 93334, 93335, 93336, 93337, 93338, 93339, 93340, 93341, 93342, 93343, 93344, 93345) c6( ) c7(oO, Kd, T, fM, BD, dA, ra, aD, jx, jh, fd, bB, C, uE, asr, V, mC, ai, x, F, hD, h, hn, ey, ji, pk, aso, hM, asn, asp, A, oV, asm, bg, Pg, eZ, Of, Og, CQ, Wt, ck, ba, ou, iQ, zr, bE, asq, lf, cj, uz, oX, k, rH, fL, hh, fD, Ze, y); #c1 (BLNK) c2(NP_001245371) c3(B-cell linker protein isoform 5) c4(93346) c5(93347, 93348, 93349, 93350, 93351, 93352, 93353, 93354, 93355, 93356, 93357) c6( ) c7(oO, Kd, T, fM, BD, dA, ra, aD, jx, jh, fd, bB, C, uE, asr, V, mC, ai, x, F, hD, h, hn, ey, ji, pk, aso, hM, asn, asp, A, oV, asm, bg, Pg, eZ, Of, Og, CO, Wt, ck, ba, ou, iQ, zr, bE, asq, lf, cj, uz, oX, k, rH, fL, hh, fD, Ze, y); #c1(BLNK) c2(NP_037446) c3(B-cell linker protein isoform 1) c4(175558) c5(175559, 175560, 175561, 175562, 175563, 175564) c6( ) c7(oO, Kd, T, fM, BD, dA, ra, aD, jx, jh, fd, bB, C, uE, asr, V, mC, ai, x, F, hD, h, hn, ey, ji, pk, aso, hM, asn, asp, A, oV, asm, bg, Pg, eZ, Of, Og, CA, Wt, ck, ba, ou, il, zr, bE, asq, lf, cj, uz, oX, k, rH, fl, hh, fD, Ze, y); #c1(BLNK) c2(XP_011538030) c3(B-cell linker protein isoform X1) c4( ) c5(335588) c6( ) c7(oO, Kd, T, fM, BD, dA, ra, aD, jx, jh, fd, bB, C, uE, asr, V, mC, ai, x, F, hD, h, hn, ey, ji, pk, aso, hM, asn, asp, A, oV, asm, bg, Pg, eZ, Of, Og, CA, Wt, ck, ba, ou, iQ, zr, bE, asq, lf, cj, uz, oX, k, rH, fL, hh, fD, Ze, y); #c1(BLOC1S2) c2(NP_001269365) c3(biogenesis of lysosome-related organelles complex 1 subunit 2 isoform 3) c4( ) c5(101289, 101290, 101291, 101292, 101293, 101294, 101295, 101296) c6( ) c7(et, HP, ag, dz, Hr, ass, aiJ); #c1(BLOC1S2) c2(NP_001269367) c3(biogenesis of lysosome-related organelles complex 1 subunit 2 isoform 2) c4( ) c5(101297, 101298, 101299, 101300, 101301) c6( ) c7(et, HP, ag, dz, Hr, ass, aiJ); #c1(BLOC1S2) c2(NP_001269368) c3(biogenesis of lysosome-related organelles complex 1 subunit 2 isoform 4) c4( ) c5(101302, 101303, 101304, 101305, 101306, 101307, 101308, 101309) c6( ) c7(et, HP, ag, dz, Hr, ass, aiJ); #c1(BLOC1S2) c2(NP_776170) c3(biogenesis of lysosome-related organelles complex 1 subunit 2 isoform 1) c4(250916, 250917, 250918) c5( ) c6( ) c7(et, HP, ag, dz, Hr, ass, aiJ); #c1(BLOC1S3) c2(NP_997715) c3(biogenesis of lysosome-related organelles complex 1 subunit 3) c4(265755) c5(265756, 265757, 265758, 265759, 265760, 265761) c6( ) c7(ast, kl, af, XF, lF); #c1(BLOC1S4) c2(NP_060836) c3(biogenesis of lysosome-related organelles complex 1 subunit 4) c4(198052) c5(198053, 198054, 198055, 198056, 198057, 198058) c6( ) c7(aC, asu, XF, lF); #c1(BLOC1S5) c2(NP_001186251) c3(biogenesis of lysosome-related organelles complex 1 subunit 5 isoform 2) c4(77451) c5(77452, 77453, 77454, 77455, 77456) c6( ) c7(IE); #c1(BLOC1S5) c2(NP_958437) c3(biogenesis of lysosome-related organelles complex 1 subunit 5 isoform 1) c4(262091) c5(262092, 262093, 262094, 262095, 262096, 262097, 262098) c6( ) c7(IE); #c1(BLOC1S6) c2(NP_036520) c3(biogenesis of lysosome-related organelles complex 1 subunit 6) c4(174220) c5(174221, 174222, 174223, 174224, 174225, 174226) c6( ) c7(DS, lE, iU, ds, lF, bw, fD, asv, Df, eA, asw); #c1(BLVRA) c2(XP_011513776) c3(biliverdin reductase A isoform X1) c4(306170, 306/71) c5(306172, 306173, 306174, 306175, 306176, 306177, 306178, 306179, 306180, 306181, 306182, 306183, 306184, 306185, 306186, 306187) c6( ) c7(dD, sO, al, U, j, K, asx, C, hJ, eq, af); #c1(BLVRB) c2(NP_000704) c3(flavin reductase (NADPH)) c4(14738) c5(14739, 14740, 14741, 14742, 14743, 14744) c6( ) c7(Ew, aoq, asy, aA, hD, aq, asA, m, asz, bw, bt, it, B); #c1(BLZF1) c2(NP_003657) c3(Golgin-45) c4(140008) c5(140009, 140010, 140011, 140012, 140013, 140014, 140015, 140016, 140017, 140018, 140019) c6( ) c7(bL, T, os, fM, Ke, asB, cz, fd, afK, oZ, fu, asF, AO, ad, bv, WY, aT, h, vc, K, jj, asC, MG, bA, hM, iU, aW, asD, rU, hl, Pg, aq, lo, kp, BG, t, asE, ck, Kc, ac, c, ll, cj, U, rK, m, cP, MC, zA, B); #c1(BLZF1) c2(XP_005245618) c3(golgin-45 isoform X2) c4(266785, 266786, 266787) c5(266788, 266789, 266790, 266791, 266792, 266793, 266794, 266795, 266796, 266797, 266798, 266799, 266800, 266801, 266802, 266803, 266804, 266805) c6( ) c7(bL, T, os, fM, Ke, asB, cz, fd, afK, oZ, fu, asF, AD, ad, bv, WY, aT, h, vc, K, jj, asC, MG, bA, hM, iU, aW, asD, rU, hl, Pg, aq, lo, kp, BG, t, asE, ck, Kc, ac, c, ll, cj, U, rK, m, cP, MC, zA, B); #c1 (BMF) c2(NP_001003942) c3(bcl-2-modifying factor isoform bmf-2) c4(20324) c5(20325, 20326, 20327, 20328, 20329, 20330) c6( ) c7(l, BN, u, eM, cj, aj, hy, ne, J); #c1(BMF) c2(NP_001003943) c3(bcl-2-modifying factor isoform bmf-3) c4(20331) c5(20332, 20333, 20334, 20335, 20336, 20337, 20338, 20339, 20340, 20341, 20342, 20343, 20344, 20345, 20346, 20347, 20348, 20349, 20350, 20351) c6( ) c7(1, BN, u, eM, cj, aj, hy, ne, J); #c1(BMF) c2(NP_277038) c3(bcl-2-modifying factor isoform bmf-1) c4(228759, 228760) c5(228761, 228762, 228763, 228764, 228765, 228766, 228767, 228768, 228769, 228770, 228771, 228772, 228773, 228774, 228775, 228776, 228777, 228778) c6( ) c7(l, BN, u, eM, cj, aj, hy, ne, J); #c1(BMI1) c2(NP_005171) c3(polycomb complex protein BMI-1) c4( ) c5(153585, 153586, 153587, 153588, 153589, 153590, 153591, 153592, 153593, 153594, 153595, 153596, 153597, 153598, 153599, 153600, 153601, 153602, 153603, 153604, 153605, 153606, 153607, 153608, 153609, 153610, 153611, 153612, 153613, 153614, 153615, 153616, 153617, 153618, 153619, 153620, 153621) c6( ) c7(T, if, fd, bB, ci, x, aE, SV, ja, K, hu, DY, pk, eL, bc, yo, aW, ark, as, eF, cm, bw, eM, m, k, ht, n, qN, br, Bl, ft, jz, eV, hn, dL, asH, dx, pl, oS, A, aq, kp, cM, ol, Qy, bP, asl, dB, hk, Qu, hL, hR, nz, it, B, bL, ul, fM, dA, yk, eX, aA, q, eA, asG, MR, aoA, aT, cy, ae, jf, Uf, iU, oV, Y, r, en, cl, t, iK, ck, QG, gX, rA, bt, kG, Mb, jh, Gn, hF, ad, av, db, d, qt, ff, eH, cr, Ej, aF, iR, bz, bE, bC, iu); #c1(BMP1) c2(NP_001190) c3(bone morphogenetic protein 1 isoform 1 precursor) c4(80503) c5( ) c6( ) c7(T, NT, bO, asM, bS, Jz, ct, j, V, eJ, le, aT, h, zp, anN, ae, af, asL, rU, ask, r, gl, Z, t, bw, eM, lp, dc, dO, asJ, pg, fD, bC, bt); #c1(BMP1) c2(NP_006120) c3(bone morphogenetic protein 1 isoform 3 precursor) c4(162214, 162215, 162216) c5(162217, 162218, 162219, 162220, 162221, 162222, 162223, 162224) c6( ) c7(T, NT, bO, asM, bS, Jz, ct, j, V, eJ, le, aT, h, zp, anN, ae, af, asl, rU, ask, r, gl, Z, t, bw, eM, lp, dc, do, asJ, pg, fD, bC, bt); #c1(BMP1) c2(XP_006716449) c3(bone morphogenetic protein 1 isoform X1) c4( ) c5(290308, 290309, 290310, 290311, 290312, 290313, 290314) c6( ) c7(T, NT, bO, asM, bS, Jz, ct, j, V, eJ, le, aT, h, zp, anN, ae, af, asL, rU, ask, r, gl, Z, t, bw, eM, lp, dc, dO, asJ, pg, fD, bC, bt); #c1 (BMP1) c2(XP_011542919) c3(bone morphogenetic protein 1 isoform X2) c4(341520) c5(341521, 341522, 341523, 341524, 341525, 341526, 341527, 341528, 341529, 341530, 341531) c6( ) c7(T, NT, bO, asM, bS, Jz, ct, j, V, eJ, le, aT, h, zp, anN, ae, af, asl, rU, ask, r, gl, Z, t, bw, eM, lp, dc, do, asJ, pg, fD, bC, bt); #c1(BMP10) c2(NP_055297) c3(bone morphogenetic protein 10 preproprotein) c4(180353) c5(180354, 180355, 180356, 180357, 180358, 180359, 180360, 180361) c6(180362, 180363) c7(aW, mx, cz, aT, ae, ad); #c1(BMP15) c2(NP_005439) c3(bone morphogenetic protein 15 precursor) c4(156184) c5(156185, 156186, 156187, 156188, 156189, 156190) c6( ) c7(UH, Ut, kl, vJ, asP, AV, aZ, il, asQ, aeS, Uv, UG, asN, asO, zZ); #c1(BMP2) c2(NP_001191) c3(bone morphogenetic protein 2 preproprotein) c4(81907, 81908, 81909) c5( ) c6( ) c7(ID, T, ea, hE, fd, bB, amj, ci, Na, bv, aE, gH, iv, zy, ly, up, HI, aW, Ls, g, eF, bw, zV, eM, acw, vA, asR, m, lS, asW, dD, wu, NT, fr, cd, asS, ai, zw, h, asT, A, iw, aq, fq, op, lx, eD, Be, B, bL, eR, aA, wo, v, j, V, pd, q, cF, afQ, ld, aT, Nc, zg, jj, ae, bA, jf, vT, oV, r, en, t, sS, ac, nG, hO, KY, hS, kh, eq, zU, xg, ad, afb, BY, abk, Ae, hP, wm, asU, d, vj, ff, eH, yy, asV, aF, iR, bz, aU, Ew, bE, adh, lC, KS, iu); #c1 (BMP2) c2(XP_011527625) c3(bone morphogenetic protein 2 isoform X1) c4(322515) c5( ) c6( ) c7(ID, T, ea, hE, fd, bB, amj, ci, Na, bv, aE, gH, iv, zy, ly, up, HI, aW, Ls, g, eF, bw, zV, eM, acw, vA, asR, m, lS, asW, dD, wu, NT, fr, cd, asS, ai, zw, h, asT, A, iw, aq, fq, op, lx, eD, Be, B, bL, eR, aA, wo, v, j, V, pd, q, cF, afQ, ld, aT, Nc, zg, jj, ae, bA, jf, vT, oV, r, en, t, sS, ac, nG, hO, KY, hS, kh, eq, zu, xg, ad, afb, BY, abk, Ae, hP, wm, asU, d, vj, ff, eH, yy, asV, aF, iR, bz, aU, Ew, bE, adh, lC, KS, iu); #c1(BMP2K) c2(NP_060063) c3(BMP-2-inducible protein kinase isoform b) c4(193962, 193963) c5(193964, 193965, 193966, 193967, 193968, 193969, 193970, 193971, 193972, 193973, 193974, 193975) c6( ) c7(dy, bA); #c1(BMP2K) c2(NP_942595) c3(BMP-2-inducible protein kinase isoform a) c4(260588) c5( ) c6( ) c7(dy, bA); #c1(BMP2K) c2(XP_011530403) c3(BMP-2-inducible protein kinase isoform X2) c4( ) c5(326248, 326249, 326250, 326251, 326252, 326253, 326254) c6( ) c7(dy, bA); #c1(BMP3) c2(XP_006714354) c3(bone morphogenetic protein 3 isoform X1) c4(287787) c5(287788, 287789, 287790, 287791, 287792, 287793) c6( ) c7(af, Ls, ln, r, Fb, q); #c1(BMP4) c2(NP_001193) c3(bone morphogenetic protein 4 preproprotein) c4(83497, 83498) c5( ) c6( ) c7(T, aC, ea, atj, bB, ci, wa, x, bv, Na, aE, ata, asY, ath, K, asX, cp, mH, li, yo, aW, Ls, hl, LZ, g, eF, iT, lp, vy, k, m, lS, fy, dD, lT, wJ, NT, zw, Lv, atc, A, kj, fl, atf, aq, Pp, dw, W, hk, w, wk, ajd, Be, hR, ati, zG, eo, aiS, B, bL, ul, eR, nx, aA, cR, cz, V, q, eA, tL, afQ, aT, Nc, zg, jj, an, ae, af, bA, kl, s, Y, ev, ate, r, t, dS, ck, Dy, ac, gX, sx, hl, qb, atk, ha, ul, eq, zU, OH, ad, dP, MS, oW, lR, xs, eH, yy, asZ, aF, bz, atb, Hv, LB, apM, yE, bE, atg, aS, y, X, atd); #c1(BMP5) c2(NP_066551) c3(bone morphogenetic protein 5 preproprotein) c4(207374) c5(207375, 207376, 207377, 207378, 207379, 207380) c6( ) c7(aW, Dd, Ls, aw, iw, ff, aT, iv, eo, en, y, V, ae, ad, dS); #c1(BMP5) c2(XP_011513118) c3(bone morphogenetic protein 5 isoform X1) c4(304909) c5(304910, 304911, 304912, 304913, 304914, 304915) c6( ) c7(aW, Dd, Ls, aw, iw, ff, aT, iv, eo, en, y, V, ae, ad, dS); #c1(BMP6) c2(NP_001709) c3(bone morphogenetic protein 6 preproprotein) c4(120305, 120306, 120307) c5(120308, 120309, 120310, 120311, 120312, 120313, 120314, 120315, 120316, 120317, 120318, 120319, 120320, 120321, 120322, 120323, 120324, 120325) c6( ) c7(T, kh, pG, aZ, jh, aA, cz, fd, aj, Fb, fr, ci, cd, J, ad, x, ib, ld, oW, kH, aT, lG, Nc, ji, K, zg, hu, ae, eu, hi, kl, A, je, aW, fJ, eH, atl, r, yy, aq, aF, eF, bz, ck, atm, iM, W, eM, cj, pg, k, lC, bt, it, B, y); #c1(BMP7) c2(NP_001710) c3(bone morphogenetic protein 7 precursor) c4(120326, 120327) c5(120328, 120329, 120330, 120331, 120332, 120333, 120334, 120335, 120336, 120337, 120338, 120339, 120340, 120341, 120342, 120343, 120344, 120345, 120346, 120347, 120348, 120349, 120350, 120351, 120352, 120353, 120354, 120355, 120356, 120357, 120358, 120359, 120360, 120361, 120362, 120363, 120364, 120365, 120366, 120367, 120368, 120369, 120370, 120371, 120372, 120373, 120374, 120375, 120376, 120377, 120378, 120379, 120380, 120381, 120382, 120383, 120384, 120385, 120386, 120387, 120388, 120389, 120390, 120391, 120392, 120393, 120394, 120395, 120396, 120397, 120398, 120399, 120400, 120401, 120402, 120403, 120404, 120405, 120406, 120407, 120408, 120409, 120410, 120411, 120412, 120413, 120414, 120415, 120416, 120417, 120418, 120419, 120420, 120421, 120422, 120423, 120424, 120425, 120426, 120427, 120428, 120429, 120430, 120431, 120432) c6(120433, 120434, 120435, 120436, 120437) c7(bL, T, kh, eR, hV, atk, fG, if, al, bO, aA, jh, aj, bX, qN, atp, aY, V, AD, q, J, ad, er, dP, fU, wa, afb, cF, aE, zw, fm, rw, lC, aT, lG, cy, ji, K, SD, atq, ae, atn, af, cp, atr, ato, A, lU, aW, aeC, Y, vj, Np, eH, r, fQ, fe, ff, Pp, uF, en, g, eF, bz, ek, iP, gk, ac, wl, X, pg, dy, Hb, lS, fy, in, B); #c1(BMP8B) c2(XP_011540326) c3(bone morphogenetic protein 88 isoform X3) c4(338764) c5(338765, 338766, 338767, 338768, 338769, 338770, 338771, 338772) c6(338773, 338774) c7(ci, vi); #c1(BMPER) c2(NP_597725) c3(BMP-binding endothelial regulator protein precursor) c4(235120) c5(235121, 235122, 235123, 235124, 235125, 235126, 235127, 235128, 235129, 235130) c6( ) c7(gk, F, VR, dc, dh, hl, oW, al, jx, h, ats, cd, vi); #c1(BMPR1A) c2(NP_004320) c3(bone morphogenetic protein receptor type-1A precursor) c4(145135) c5(145136, 145137, 145138, 145139, 145140, 145141, 145142, 145143, 145144, 145145) c6( ) c7(bL, lD, T, jW, aZ, atv, al, NT, aA, v, Cd, Cb, eA, ad, er, Cj, rw, BJ, bj, atw, K, ae, Ur, wC, ly, B, Yd, Az, atu, atf, aF, att, op, lx, bF, nM, vA, gX, adh, lC, X); #c1(BMPR1B) c2(NP_001243721) c3(bone morphogenetic protein receptor type-18 isoform b precursor) c4(91547) c5(91548, 91549, 91550, 91551, 91552, 91553, 91554, 91555, 91556, 91557, 91558, 91559, 91560, 91561, 91562, 91563, 91564, 91565, 91566, 91567, 91568, 91569, 91570, 91571, 91572, 91573, 91574, 91575) c6( ) c7(bA, bM, atx, T, aty, d, lD, aW, BJ, ln, Az, NT, aT, cz, zU, bX, lC, Qx, atb, ad, BY); #c1(BMPR1B) c2(NP_001243722) c3(bone morphogenetic protein receptor type-18 isoform a precursor) c4(91576, 91577, 91578) c5(91579, 91580, 91581, 91582, 91583, 91584, 91585, 91586, 91587, 91588, 91589, 91590) c6( ) c7(bA, bM, atx, T, aty, d, lD, aW, BJ, ln, Az, NT, aT, cz, zU, bX, lC, Qx, atb, ad, BY); #c1(BMPR2) c2(NP_001195) c3(bone morphogenetic protein receptor type-2 precursor) c4(83499) c5(83500, 83501, 83502, 83503, 83504, 83505) c6( ) c7(ID, bM, T, atB, atC, e, al, NT, cz, bB, v, j, ci, lR, fr, q, ad, BY, XX, Cj, Zs, lC, yg, aT, h, lG, BZ, atD, K, uV, bq, rz, ae, wC, ly, ue, atA, eH, r, eF, pb, lx, bE, wl, gZ, uo, aS, atz, jr); #c1(BMPR2) c2(XP_011509989) c3(bone morphogenetic protein receptor type-2 isoform X1) c4(300604) c5( ) c6( ) c7(ID, bM, T, atB, atC, e, al, NT, cz, bB, v, j, ci, lR, fr, q, ad, BY, XX, Cj, Zs, lC, yg, aT, h, lG, BZ, atD, K, uV, bq, rz, ae, wC, ly, ue, atA, eH, r, eF, pb, lx, bE, wl, gZ, uo, aS, atz, jr); #c1(BMPR2) c2(XP_011509990) c3(bone morphogenetic protein receptor type-2 isoform X2) c4( ) c5(300605, 300606, 300607, 300608, 300609, 300610) c6( ) c7(ID, bM, T, atB, atC, e, al, NT, cz, bB, v, j, ci, lR, fr, q, ad, BY, XX, Cj, Zs, lC, yg, aT, h, lG, BZ, atD, K, uV, bq, rz, ae, wC, ly, ue, atA, eH, r, eF, pb, lx, bE, wl, gZ, uo, aS, atz, jr); #c1(BMS1) c2(NP_055568) c3(ribosome biogenesis protein BMS1 homolog) c4(181975) c5(181976, 181977, 181978, 181979, 181980, 181981) c6( ) c7(bL, T, eR, aD, qB, cz, fF, n, cL, bS, j, ci, v, aY, ad, o, qH, qC, dP, ft, ib, qK, iv, jy, qG, ae, af, hm, qE, iU, no, iw, cr, qL, eZ, mv, cm, qJ, ql, act, qF, i, hS); #c1(BMX) c2(NP_001712) c3 (cytoplasmic tyrosine-protein kinase BMX) c4(120438) c5(120439, 120440, 120441, 120442, 120443, 120444, 120445, 120446, 120447, 120448, 120449, 120450, 120451, 120452) c6( ) c7(ae, T, ft, aW, aT, h, bw, fr, y, bz, ad); #c1(BNC1) c2(NP_001288135) c3(zinc finger protein basonuclin-1 isoform b) c4(113594) c5(113595, 113596, 113597, 113598, 113599, 113600) c6( ) c7(T, sS, aE, ff, aq, en, B); #c1(BNC1) c2(NP_001708) c3(zinc finger protein basonuclin-1 isoform a) c4(120297) c5(120298, 120299, 120300, 120301, 120302, 120303, 120304) c6( ) c7(T, sS, aE, ff, aq, en, B); #c1(BNC1) c2(XP_011520196) c3(zinc finger protein basonuclin-1 isoform X2) c4( ) c5(315081) c6( ) c7(T, sS, aE, ff, aq, en, B); #c1(BNC2) c2(NP_060107) c3(zinc finger protein basonuclin-2) c4(194189, 194190) c5(194191, 194192, 194193, 194194, 194195, 194196, 194197, 194198, 194199, 194200, 194201, 194202, 194203, 194204, 194205, 194206, 194207, 194208, 194209, 194210, 194211, 194212, 194213, 194214, 194215, 194216, 194217, 194218, 194219) c6( ) c7(dP, Na, A, pf, bo, Az, aj, Pp, m, t, J); #c1(BNC2) c2(XP_011516223) c3(zinc finger protein basonuclin-2 isoform X1) c4(309441, 309442, 309443) c5(309444) c6( ) c7(dP, Na, A, pf, bo, Az, aj, Pp, m, t, J); #c1(BNC2) c2(XP_011516224) c3(zinc finger protein basonuclin-2 isoform X2) c4( ) c5(309445, 309446, 309447, 309448, 309449, 309450) c6( ) c7(dP, Na, A, pf, bo, Az, aj, Pp, m, t, J); #c1(BNC2) c2(XP_011516226) c3(zinc finger protein basonuclin-2 isoform X4) c4(309451) c5( ) c6( ) c7(dP, Na, A, pf, bo, Az, aj, Pp, m, t, J); #c1(BNC2) c2(XP_011516229) c3(zinc finger protein basonuclin-2 isoform X7) c4(309452) c5( ) c6( ) c7(dP, Na, A, pf, bo, Az, aj, Pp, m, t, J); #c1(BNC2) c2(XP_011516230) c3(zinc finger protein basonuclin-2 isoform X8) c4( ) c5(309453, 309454, 309455, 309456, 309457, 309458, 309459, 309460, 309461, 309462, 309463, 309464, 309465, 309466, 309467, 309468) c6( ) c7(dP, Na, A, pf, bo, Az, aj, Pp, m, t, J); #c1(BNC2) c2(XP_011516234) c3(zinc finger protein basonuclin-2 isoform X9) c4( ) c5(309469, 309470, 309471, 309472, 309473, 309474) c6( ) c7(dP, Na, A, pf, bo, Az, aj, Pp, m, t, J); #c1(BNIP1) c2(NP_001196) c3(vesicle transport protein SEC20 isoform BNIP1) c4(83506) c5(83507, 83508, 83509, 83510, 83511, 83512) c6( ) c7(aeP, aW, cz, ad, aT); #c1(BNIP1) c2(NP_053581) c3(vesicle transport protein SEC20 isoform BNIP1-a) c4(177216) c5(177217, 177218, 177219, 177220, 177221, 177222) c6( ) c7(aeP, aW, cz, ad, aT); #c1(BNIP1) c2(NP_053582) c3(vesicle transport protein SEC20 isoform BNIP1-b) c4(177223) c5(177224, 177225, 177226, 177227, 177228, 177229) c6( ) c7(aeP, aW, cz, ad, aT); #c1(BNIP1) c2(NP_053583) c3(vesicle transport protein SEC20 isoform BNIP1-c) c4(177230) c5(177231, 177232, 177233, 177234, 177235, 177236) c6( ) c7(aeP, aW, cz, ad, aT); #c1(BNIP2) c2(NP_004321) c3(BCL2/adenovirus E1B 19 kDa protein-interacting protein 2) c4(145146, 145147) c5(145148, 145149, 145150, 145151, 145152, 145153, 145154, 145155, 145156, 145157, 145158, 145159) c6( ) c7(cm, iW, ae, cF, ad); #c1(BNIP3) c2(NP_004043) c3(BCL2/adenovirus E1B 19 kDa protein-interacting protein 3) c4(143102, 143103) c5(143104, 143105, 143106, 143107, 143108, 143109, 143110, 143111, 143112, 143113, 143114, 143115) c6( ) c7(T, aC, fM, rQ, iW, aw, aA, fd, n, fr, ci, q, cd, ad, eA, x, av, aE, aT, Lv, cy, K, ae, d, aW, Y, r, QP, aq, vs, aF, g, ck, es, ac, eM, iA, bE, gX, wz, gZ, pg, qF, B); #c1(BNIP3L) c2(NP_004322) c3(BCL2/adenovirus E1B 19 kDa protein-interacting protein 3-like) c4(145160, 145161) c5(145162, 145163, 145164, 145165, 145166, 145167, 145168, 145169, 145170, 145171, 145172, 145173, 145174, 145175, 145176) c6( ) c7(ar, T, BN, pc, aE, aW, aT, gZ, cy, cd, aj, hy, K, m, an, t, ae, ne, J, ad); #c1(BNIP3L) c2(XP_011542932) c3(BCL2/adenovirus E1B 19 kDa protein-interacting protein 3-like isoform X1) c4(341568) c5(341569, 341570, 341571, 341572, 341573, 341574, 341575, 341576, 341577, 341578, 341579) c6( ) c7(ar, T, BN, pc, aE, aW, aT, gZ, cy, cd, aj, hy, K, m, an, t, ae, ne, J, ad); #c1(BNIPL) c2(XP_011507538) c3(bcl-2/adenovirus E1B 19 kDa-interacting protein 2-like protein isoform X1) c4( ) c5(297468, 297469) c6( ) c7(B); #c1(BOC) c2(NP_001288790) c3(brother of CDO isoform 1 precursor) c4(114044) c5(114045, 114046, 114047, 114048, 114049, 114050, 114051, 114052, 114053, 114054, 114055) c6( ) c7(jf, gV, aiw, hG); #c1(BOC) c2(NP_001288796) c3(brother of CDO isoform 3 precursor) c4(114056) c5(114057, 114058, 114059, 114060, 114061, 114062, 114063, 114064, 114065, 114066, 114067, 114068, 114069, 114070, 114071, 114072, 114073, 114074, 114075, 114076, 114077, 114078, 114079, 114080, 114081, 114082) c6( ) c7(jf, gV, aiw, hG); #c1(BOC) c2(XP_005247954) c3(brother of CDO isoform X5) c4(268153, 268154, 268155) c5( ) c6( ) c7(jf, gV, aiw, hG); #c1(BOC) c2(XP_011511607) c3(brother of CDO isoform X2) c4(302332) c5(302333, 302334, 302335, 302336, 302337, 302338, 302339, 302340, 302341, 302342) c6( ) c7(jf, gV, aiw, hG); #c1(BOC) c2(XP_011511608) c3(brother of CDO isoform X3) c4( ) c5(302343, 302344) c6( ) c7(jf, gV, aiw, hG); #c1(BOD1L2) c2(NP_001244893) c3(biorientation of chromosomes in cell division protein 1-like 2) c4(92259, 92260) c5(92261, 92262, 92263, 92264, 92265, 92266, 92267, 92268, 92269, 92270, 92271, 92272) c6( ) c7(j); #c1(BOK) c2(NP_115904) c3(bcl-2-related ovarian killer protein) c4(224595) c5(224596, 224597, 224598, 224599, 224600, 224601) c6( ) c7(hb, uC, ul, id); #c1(BOLA3) c2(NP_001030582) c3(bolA-like protein 3 isoform 2) c4(31027) c5(31028, 31029, 31030, 31031, 31032, 31033) c6( ) c7(atE, UZ, atF); #c1(BOLA3) c2(NP_997717) c3(bolA-like protein 3 isoform 1) c4(265762) c5(265763, 265764, 265765, 265766, 265767, 265768) c6( ) c7(atE, UZ, atF); #c1(BOLL) c2(NP_001271287) c3(protein boule-like isoform 3) c4(103848) c5(103849, 103850, 103851, 103852, 103853, 103854) c6( ) c7(gU, ND, aec, vH, g, us, dR); #c1(BOLL) c2(NP_001271291) c3(protein boule-like isoform 5) c4( ) c5(103855) c6( ) c7(gU, ND, aec, vH, g, us, dR); #c1(BOLL) c2(NP_149019) c3(protein boule-like isoform 2) c4( ) c5(226776, 226777, 226778, 226779, 226780, 226781, 226782, 226783) c6( ) c7(gU, ND, aec, vH, g, us, dR); #c1(BOLL) c2(NP_932074) c3(protein boule-like isoform 1) c4(258806, 258807) c5(258808, 258809, 258810, 258811, 258812, 258813, 258814) c6( ) c7(gU, ND, aec, vH, g, us, dR); #c1(BOPI) c2(NP_056016) c3(ribosome biogenesis protein BOP1) c4(185151, 185152) c5(185153, 185154, 185155, 185156, 185157, 185158, 185159, 185160, 185161, 185162, 185163, 185164) c6( ) c7(NT, aA); #c1(BORA) c2(NP_001273675) c3(protein aurora borealis isoform 2) c4(105595) c5(105596, 105597, 105598, 105599, 105600) c6( ) c7(fq); #c1(BORA) c2(NP_001273676) c3(protein aurora borealis isoform 3) c4(105601) c5(105602, 105603, 105604, 105605, 105606, 105607) c6( ) c7(fq); #c1(BORA) c2(NP_079084) c3(protein aurora borealis isoform 1) c4( ) c5(216929) c6( ) c7(fq); #c1(BPGM) c2(NP_001280014) c3(bisphosphoglycerate mutase) c4(111236, 111237) c5(111238, 111239, 111240, 111241, 111242, 111243, 111244, 111245, 111246, 111247, 111248, 111249, 111250, 111251, 111252, 111253, 111254, 111255, 111256, 111257) c6( ) c7(atG, atH, bK, atl); #c1(BPI) c2(NP_001716) c3(bactericidal permeability-increasing protein precursor) c4(120460, 120461, 120462, 120463, 120464) c5(120465, 120466, 120467, 120468, 120469, 120470) c6( ) c7(oO, bL, am, fR, aw, al, mS, bS, ct, C, mW, dL, bK, ji, mY, he, mV, N, aa, mU, atJ, bg, hl, fe, aga, iV, Z, mZ, pb, DF, mX, gk, eM, at, Zn, uJ, mf, y, mT, ao); #c1(BPI) c2(XP_005260579) c3 (bactericidal permeability-increasing protein isoform X1) c4(276640) c5(276641, 276642, 276643, 276644, 276645, 276646, 276647, 276648, 276649, 276650, 276651, 276652, 276653, 276654, 276655, 276656, 276657, 276658, 276659, 276660, 276661, 276662, 276663, 276664, 276665, 276666, 276667, 276668, 276669, 276670, 276671, 276672, 276673, 276674, 276675, 276676, 276677, 276678, 276679, 276680, 276681, 276682, 276683, 276684, 276685, 276686, 276687, 276688, 276689, 276690, 276691, 276692, 276693, 276694, 276695, 276696, 276697, 276698, 276699, 276700, 276701, 276702, 276703, 276704, 276705, 276706, 276707, 276708, 276709, 276710, 276711, 276712, 276713, 276714, 276715, 276716, 276717, 276718, 276719, 276720, 276721, 276722, 276723, 276724, 276725, 276726, 276727) c6(276728) c7(oO, bL, am, fR, aw, al, mS, bS, ct, C, mW, dL, bK, ji, mY, he, mV, N, aa, mU, atJ, bg, hl, fe, aga, iV, Z, mZ, pb, DF, mX, gk, eM, at, Zn, uJ, mf, y, mT, ao); #c1(BPIFA1) c2(NP_057667) c3(BPI fold-containing family A member 1 precursor) c4(192226, 192227) c5(192228, 192229, 192230, 192231, 192232, 192233, 192234, 192235, 192236, 192237, 192238, 192239, 192240, 192241, 192242, 192243, 192244, 192245, 192246, 192247, 192248, 192249, 192250, 192251, 192252, 192253, 192254, 192255) c6(192256, 192257, 192258, 192259, 192260, 192261, 192262, 192263) c7(oO, av, eM, afm, df, ff, n, zV, ci, en, g, zy, B, ao, aa); #c1(BPIFA2) c2(NP_542141) c3(BPI fold-containing family A member 2 precursor) c4(231396) c5(231397, 231398, 231399, 231400, 231401, 231402, 231403, 231404, 231405, 231406, 231407, 231408, 231409, 231410, 231411) c6(231412, 231413, 231414, 231415) c7(ar, kp, act, lB, ae, ad); #c1(BPIFA3) c2(NP_001035904) c3(BPI fold-containing family A member 3 isoform 2 precursor) c4(33358) c5(33359, 33360, 33361, 33362, 33363, 33364, 33365, 33366) c6(33367, 33368) c7(oe); #c1(BPIFA3) c2(NP_848561) c3(BPI fold-containing family A member 3 isoform 1 precursor) c4(253860) c5(253861, 253862, 253863, 253864, 253865, 253866, 253867, 253868) c6(253869, 253870) c7(de); #c1(BPIFB1) c2(NP_149974) c3(BPI fold-containing family B member 1 precursor) c4(227681, 227682, 227683) c5(227684, 227685, 227686, 227687, 227688, 227689, 227690, 227691, 227692, 227693, 227694, 227695, 227696, 227697, 227698, 227699, 227700, 227701, 227702, 227703, 227704, 227705, 227706, 227707) c6(227708, 227709, 227710, 227711, 227712, 227713) c7(bW, jN, uo, ci, yb, OM, atK, ao); #c1(BPIFB2) c2(NP_079503) c3(BPI fold-containing family B member 2 precursor) c4(218418) c5(218419, 218420, 218421, 218422, 218423, 218424, 218425, 218426) c6(218427, 218428) c7(atL); #c1(BPIFC) c2(NP_777592) c3(BPI fold-containing family C protein precursor) c4(251264) c5(251265, 251266, 251267, 251268, 251269, 251270, 251271, 251272, 251273, 251274, 251275, 251276, 251277, 251278, 251279) c6(251280, 251281, 251282, 251283) c7(m); #c1(BPIFC) c2(XP_011528393) c3(BPI fold-containing family C protein isoform X2) c4(323480) c5(323481) c6( ) c7(m); #c1(BPIFC) c2(XP_011528395) c3(BPI fold-containing family C protein isoform X3) c4(323482) c5(323483, 323484, 323485, 323486, 323487, 323488, 323489, 323490, 323491, 323492, 323493, 323494, 323495, 323496) c6(323497, 323498, 323499, 323500) c7(m); #c1(BPNT1) c2(NP_001273078) c3(3′(2′),5′-bisphosphate nucleotidase 1 isoform 2) c4( ) c5(104294, 104295, 104296, 104297, 104298) c6( ) c7(T, KW, Fb, fr, zF, ci, nA, atN, ai, ad, aT, DO, K, ae, aW, Ue, zD, Pp, t, atM, dB, zB); #c1(BPNT1) c2(NP_001273079) c3(3′(2′),5′-bisphosphate nucleotidase 1 isoform 3) c4(104299) c5(104300, 104301, 104302, 104303, 104304, 104305) c6( ) c7(T, KW, Fb, fr, zF, ci, nA, atN, ai, ad, aT, DO, K, ae, aW, Ue, zD, Pp, t, atM, dB, zB); #c1(BPNT1) c2(NP_006076) c3(3′(2′),5′-bisphosphate nucleotidase 1 isoform 1) c4(161694, 161695, 161696) c5(161697, 161698, 161699, 161700, 161701, 161702, 16/703, 161704, 161705, 161706, 161707, 161708, 161709, 161710, 161711, 161712, 16/713) c6( ) c7(T, KW, Fb, fr, zF, ci, nA, atN, ai, ad, aT, DO, K, ae, aW, Ue, zD, Pp, t, atM, dB, zB); #c1(BPNT1) c2(XP_005273056) c3(3′(2′),5′-bisphosphate nucleotidase 1 isoform X2) c4(284284) c5( ) c6( ) c7(T, KW, Fb, fr, zF, ci, nA, atN, ai, ad, aT, DO, K, ae, aW, Ue, zD, Pp, t, atM, dB, zB); #c1(BPNT1) c2(XP_011507367) c3(3′(2′),5′-bisphosphate nucleotidase 1 isoform X7) c4( ) c5(297207, 297208, 297209, 297210, 297211, 297212) c6( ) c7(T, KW, Fb, fr, zF, ci, nA, atN, ai, ad, aT, DO, K, ae, aW, Ue, zD, Pp, t, atM, dB, zB); #c1(BPTF) c2(NP_004450) c3(nucleosome-remodeling factor subunit BPTF isoform 2) c4(146411) c5(146412, 146413, 146414, 146415, 146416, 146417, 146418, 146419, 146420, 146421, 146422, 146423, 146424) c6( ) c7(aW, dL, aT); #c1 (BPTF) c2(NP_872579) c3(nucleosome-remodeling factor subunit BPTF isoform 1) c4(256411) c5(256412, 256413, 256414, 256415, 256416, 256417) c6( ) c7(aW, dL, aT); #c1(BPTF) c2(XP_005257207) c3(nucleosome-remodeling factor subunit BPTF isoform X1) c4( ) c5(274756, 274757, 274758, 274759, 274760, 274761, 274762, 274763, 274764, 274765, 274766, 274767) c6( ) c7(aW, dl, aT); #c1(BPTF) c2(XP_005257210) c3(nucleosome-remodeling factor subunit BPTF isoform X5) c4( ) c5(274768, 274769, 274770, 274771, 274772, 274773) c6( ) c7(aW, dL, aT); #c1(BPTF) c2(XP_005257211) c3(nucleosome-remodeling factor subunit BPTF isoform X6) c4(274774, 274775) c5( ) c6( ) c7(aW, dL, aT); #c1(BPTF) c2(XP_005257212) c3(nucleosome-remodeling factor subunit BPTF isoform X8) c4(274776) c5(274777, 274778, 274779, 274780, 274781, 274782) c6( ) c7(aW, dL, aT); #c1(BPTF) c2(XP_005257217) c3 (nucleosome-remodeling factor subunit BPTF isoform X15) c4( ) c5(274783) c6( ) c7(aW, dL, aT); #c1(BPTF) c2(XP_005257218) c3(nucleosome-remodeling factor subunit BPTF isoform X16) c4(274784) c5( ) c6( ) c7(aW, dL, aT); #c1(BPTF) c2(XP_005257219) c3(nucleosome-remodeling factor subunit BPTF isoform X18) c4( ) c5(274785, 274786) c6( ) c7(aW, dL, aT); #c1(BPTF) c2(XP_011522826) c3(nucleosome-remodeling factor subunit BPTF isoform X17) c4(317314, 317315, 317316) c5(317317, 317318, 317319, 317320, 317321, 317322) c6( ) c7(aW, dL, aT); #c1 (BPTF) c2(XP_011522828) c3(nucleosome-remodeling factor subunit BPTF isoform X20) c4( ) c5(317323, 317324, 317325, 317326, 317327) c6( ) c7(aW, dL, aT); #c1(BPY2) c2(NP_004669) c3(testis-specific basic protein Y 2) c4(148696) c5(148697, 148698, 148699, 148700, 148701, 148702, 148703, 148704, 148705, 148706, 148707, 148708, 148709, 148710, 148711, 148712) c6( ) c7(ae, gU, ad, ND); #c1(BRAF) c2(NP_004324) c3(serine/threonine-protein kinase B-raf) c4(145177) c5(145178, 145179, 145180, 145181, 145182, 145183) c6( ) c7(DS, atT, T, aC, apr, aiR, yS, if, WV, fd, bB, ci, aub, aon, auu, Qp, ip, Vp, afr, bv, xy, aE, pX, aeO, Qk, auB, iv, auf, K, MO, Zm, cp, YL, xQ, DA, eG, yo, auQ, aW, Ls, QB, Az, XV, nY, Qq, Qv, CT, NR, apm, g, as, cm, Qt, iP, qJ, eY, atU, auD, eM, nB, aep, cj, [w, k, m, ik, iu, atO, sc, Uc, wu, sw, zX, RX, ht, NT, iB, n, kv, fr, qN, rP, auA, atV, atW, au, auv, ft, jz, hw, auj, i, Nn, gd, KN, h, aul, By, dL, aux, yT, auS, Xh, Lg, Fp, kj, aua, auM, PX, iw, ks, aq, agF, kp, po, jT, cM, auC, XT, Zj, ar, VZ, bP, ky, aul, dB, hk, eD, hL, auh, aum, aur, hR, nL, auR, bD, it, B, AN, fM, AS, aA, hz, Qo, auG, q, eA, zn, RU, aud, auf, aup, aul, nv, nX, atQ, aT, aut, Fx, atZ, ae, ne, jf, Uf, s, vT, NE, oV, Y, auc, ev, iU, r, auk, en, bu, cl, LV, t, ho, apx, iK, ck, aug, sS, nM, Dy, auo, auz, aui, np, auN, gT, bt, On, Mb, id, atY, qz, iW, atS, VT, jh, KO, auq, atR, ah, Fb, Fc, anu, aue, ad, atP, dP, av, hd, agd, ib, db, yt, oz, ds, NS, aun, hP, auE, wm, fb, pq, atX, Yj, d, lx, BO, asf, aus, ff, nK, auP, aF, VI, iV, adj, bz, Hp, bF, es, auJ, amS, Ew, bE, Lj, auw, auH, Lb, auk, adg, y, xg, X, jr, auy); #c1(BRAP) c2(NP_006759) c3(BRCAl-associated protein) c4(167798, 167799) c5(167800, 167801, 167802, 167803, 167804, 167805, 167806, 167807, 167808, 167809, 167810, 167811) c6( ) c7(bL, Si, T, dD, pF, ck, aW, e, aw, aT, auT, j, bK, ei, eq, add); #c1(BRAT1) c2(NP_689956) c3(BRCA1-associated ATM activator 1) c4(244524) c5(244525, 244526, 244527, 244528, 244529, 244530) c6( ) c7(ha, auU, lQ); #c1(BRCA1) c2(NP_009225) c3(breast cancer type 1 susceptibility protein isoform 1) c4(171607, 171608, 171609, 171610, 171611) c5(171612, 171613, 171614, 171615, 171616, 171617, 171618, 171619, 171620, 171621, 171622, 171623, 171624, 171625, 171626, 171627, 171628, 171629, 171630, 171631, 171632, 171633, 171634, 171635, 171636, 171637, 171638, 171639, 171640, 171641, 171642, 171643, 171644, 171645, 171646, 171647, 171648, 171649, 171650, 171651, 171652, 171653, 171654, 171655) c6( ) c7(ng, atT, os, T, aC, auW, ea, xo, e, if, fd, bB, aeu, gf, ci, Jt, ip, gJ, aE, Ma, Xc, il, ajv, avd, gs, K, hu, MO, eL, avn, bc, avq, ave, xQ, zQ, wp, LC, aW, Qm, g, ir, eF, cm, aj, iP, eM, lp, mx, cj, cX, avo, m, in, iu, dD, sw, aw, ht, n, To, aj, fr, ct, bd, qN, zP, cd, ajL, Bl, no, hw, zf, gd, KN, cU, h, eV, dL, asT, on, pl, Yl, ia, avc, hT, aeU, hl, agF, kp, mo, ajB, avf, zZ, QK, hq, Qy, auV, bP, dB, hk, anZ, PI, avk, Ei, hL, Vy, at, avr, qF, nz, WL, it, B, DM, dA, Dh, afl, avm, eX, ew, Nt, cR, aA, cz, hy, v, C, bX, AD, V, q, J, er, eA, eJ, mj, cF, amg, MR, hW, afQ, BJ, eD, qK, aT, qv, avt, avl, ae, af, qw, avg, arl, wr, s, iU, oV, Y, PL, aeS, r, aux, avh, eS, en, avb, cl, t, iK, aoy, ck, nM, kP, Dy, Li, hr, fD, im, bt, ava, avi, ao, gV, GI, kh, hl, id, avj, aZ, iW, jh, oZ, bS, Gn, eq, MC, avv, ad, afb, hX, dP, avu, avp, ib, db, yt, vJ, avs, qh, oW, bj, xp, ji, aeI, auY, d, BN, lx, Ub, BO, PE, Hh, hG, qt, ff, aF, Z, bz, dr, lU, bF, Ew, bE, auZ, X, WK, afd, aS, bC, be, ei, hE, aed, kS, ne); #c1(BRCA1) c2(NP_009228) c3(breast cancer type 1 susceptibility protein isoform 3) c4(171656, 171657, (71658) c5(171659, 171660, 171661, 171662, 171663, 171664) c6( ) c7(ng, atT, os, T, aC, auW, ea, xo, e, if, fd, bB, aeu, gf, ci, Jt, ip, gJ, aE, Ma, Xc, il, ajv, avd, gs, K, hu, MO, eL, avn, bc, avq, ave, xQ, zQ, wp, LC, aW, Qm, g, ir, eF, cm, aj, iP, eM, lp, mx, cj, cX, avo, m, in, iu, dD, sw, aw, ht, n, To, aj, fr, ct, bd, qN, zP, cd, ajL, Bl, no, hw, zf, gd, KN, cU, h, eV, dL, asT, on, pl, Yl, ia, avc, hT, aeI, hl, agF, kp, mo, ajB, avf, zZ, QK, hq, Qy, auV, bP, dB, hk, anZ, PI, avk, Ei, hL, Vy, at, avr, qF, nz, WL, it, B, DM, dA, Dh, afl, avm, eX, ew, Nt, CR, aA, cz, hy, v, C, bX, AO, V, q, J, er, eA, eJ, mj, cF, amg, MR, hW, afQ, BJ, eO, qK, aT, qv, avt, avl, ae, af, qw, avg, arl, wr, s, iU, oV, Y, PL, aeS, r, aux, avh, eS, en, avb, cl, t, ik, aoy, ck, nM, kP, Dy, Li, hr, fD, im, bt, ava, avi, ao, gV, Gl, kh, hl, id, avj, aZ, iW, jh, oZ, bS, Gn, eq, MC, avv, ad, afb, hX, dP, avu, avp, ib, db, yt, vJ, avs, qh, oW, bj, xp, ji, aeI, auY, d, BN, lx, Ub, BO, PE, Hh, hG, qt, ff, aF, Z, bz, dr, lU, bF, Ew, bE, auZ, X, WK, afd, aS, bC, be, ei, hE, aed, kS, ne); #c1(BRCA1) c2(NP_009229) c3(breast cancer type 1 susceptibility protein isoform 4) c4(171665, 171666, 171667, 171668, 171669, 171670, 171671) c5(171672, 171673, 171674, 171675, 171676, 171677, 171678, 171679, 171680, 171681, 171682, 171683, 171684, 171685, 171686, 171687, 171688, 171689, 171690, 171691, 171692, 171693, 171694, 171695) c6( ) c7(ng, atT, os, T, aC, auW, ea, xo, e, if, fd, bB, aeu, gf, ci, Jt, ip, gJ, aE, Ma, Xc, il, ajv, avd, gs, K, hu, MO, eL, avn, bc, avq, ave, xQ, zQ, wp, LC, aW, Qm, g, ir, eF, cm, aj, iP, eM, lp, mx, cj, cX, avo, m, in, iu, dD, sw, aw, ht, n, To, aj, fr, ct, bd, qN, zP, cd, ajL, Bl, no, hw, zf, gd, KN, cU, h, eV, dL, asT, on, pl, Yl, ia, avc, hT, aeI, hQ, agF, kp, mo, ajB, avf, zZ, QK, hq, Qy, auV, bP, dB, hk, anZ, PI, avk, Ei, hL, Vy, at, avr, qF, nz, WL, it, B, DM, dA, Dh, afl, avm, eX, ew, Nt, CR, aA, cz, hy, v, C, bX, AO, V, q, J, er, eA, eJ, mj, cF, amg, MR, hW, afQ, BJ, eO, qK, aT, qv, avt, avl, ae, af, qw, avg, arl, wr, s, iU, oV, Y, PL, aeS, r, aux, avh, eS, en, avb, cl, t, iK, aoy, ck, nM, kP, Dy, Li, hr, fD, im, bt, ava, avi, ao, gV, GI, kh, hl, id, avj, aZ, iW, jh, oZ, bS, Gn, eq, MC, avv, ad, afb, hX, dP, avu, avp, ib, db, yt, vJ, avs, qh, oW, bj, xp, ji, aeI, auY, d, BN, lx, Ub, BO, PE, Hh, hG, qt, ff, aF, Z, bz, dr, lU, bF, Ew, bE, auZ, X, WK, afd, aS, bC, be, ei, hE, aed, kS, ne); #c1(BRCA1) c2(NP_009230) c3(breast cancer type 1 susceptibility protein isoform 5) c4(171696) c5(171697, 171698, 171699, 171700, 171701, 171702, 171703, 171704, 171705, 171706, 171707, 171708, 171709, 171710, 171711, 171712, 171713, 171714, 171715, 171716, 171717, 171718, 171719, 171720, 171721, 17/722, 171723, 171724, 171725, 171726, 171727, 171728, 171729, 171730, 171731, 171732, 171733, 171734, 171735, 171736, 171737, 171738, 171739, 171740, 171741, 171742, 171743, 171744, 17/745) c6( ) c7(ng, atT, os, T, aC, auW, ea, xo, e, if, fd, bB, aeu, gf, ci, Jt, ip, gJ, aE, Ma, Xc, il, ajv, avd, gs, K, hu, MO, eL, avn, bc, avq, ave, xQ, zQ, wp, LC, aW, Qm, g, ir, eF, cm, aj, iP, eM, lp, mx, cj, cX, avo, m, in, iu, dD, sw, aw, ht, n, To, aj, fr, ct, bd, qN, zP, cd, ajL, Bl, no, hw, zf, gd, KN, cU, h, eV, dL, asT, on, pl, Yl, ia, avc, hT, aeU, hQ, agF, kp, mo, ajB, avf, zZ, QK, hq, Qy, auV, bP, dB, hk, anZ, PI, avk, Ei, hL, Vy, at, avr, qF, nz, WL, it, B, DM, dA, Dh, afl, avm, eX, ew, Nt, CR, aA, cz, hy, v, C, bX, AD, V, q, J, er, eA, eJ, mj, cF, amg, MR, hW, afQ, BJ, eO, qK, aT, qv, avt, avl, ae, af, qw, avg, arl, wr, s, iU, oV, Y, PL, aeS, r, aux, avh, eS, en, avb, cl, t, ik, aby, ck, nM, kP, Dy, Li, hr, fD, im, bt, ava, avi, ao, gV, GI, kh, hl, id, avj, aZ, iW, jh, oZ, bS, Gn, eq, MC, avv, ad, afb, hX, dP, avu, avp, ib, db, yt, vJ, avs, qh, oW, bj, xp, ji, aeI, auY, d, BN, lx, Ub, BO, PE, Hh, hG, qt, ff, aF, Z, bz, dr, lU, bF, Ew, bE, auZ, X, WK, afd, aS, bC, be, ei, hE, aed, KS, ne); #c1 (BRCA1) c2(NP_009231) c3(breast cancer type 1 susceptibility protein isoform 2) c4(171746, 171747, 171748, 171749, 171750, 171751, 171752) c5(171753, 171754, 171755, 171756, 171757, 171758, 171759, 171760, 171761, 171762, 171763, 171764, 171765, 171766, 171767, (71768, 17/769, 171770, 171771) c6( ) c7(ng, atT, os, T, aC, auW, ea, xo, e, if, fd, bB, aeu, gf, ci, Jt, ip, gJ, aE, Ma, Xc, il, ajv, avd, gs, K, hu, MO, eL, avn, bc, avq, ave, xQ, zQ, wp, LC, aW, Qm, g, ir, eF, cm, aj, iP, eM, lp, mx, cj, cX, avo, m, in, iu, dD, sw, aw, ht, n, To, aj, fr, ct, bd, qN, zP, cd, ajL, Bl, no, hw, zf, gd, KN, cU, h, eV, dL, asT, on, pl, Yl, ia, avc, hT, aeI, hQ, agF, kp, mo, ajB, avf, zZ, QK, hq, Dy, auV, bP, dB, hk, anZ, PI, avk, Ei, hL, Vy, at, avr, qF, nz, WL, it, B, DM, dA, Dh, afl, avm, eX, ew, Nt, CR, aA, cz, hy, v, C, bX, AD, V, q, J, er, eA, eJ, mj, cF, amg, MR, hW, afQ, BJ, eO, qK, aT, qv, avt, avl, ae, af, qw, avg, arl, wr, s, iU, oV, Y, PL, aeS, r, aux, avh, eS, en, avb, cl, t, ik, aoy, ck, nM, kP, Dy, Li, hr, fD, im, bt, ava, avi, ao, gV, Gl, kh, hl, id, avj, aZ, iW, jh, oZ, bS, Gn, eq, MC, avv, ad, afb, hX, dP, avu, avp, ib, db, yt, vJ, avs, qh, oW, bj, xp, ji, aeQ, auY, d, BN, lx, Ub, BO, PE, Hh, hG, qt, ff, aF, Z, bz, dr, lU, bF, Ew, bE, auZ, X, WK, afd, aS, bC, be, ei, hE, aed, KS, ne); #c1(BRCA2) c2(NP_000050) c3(breast cancer type 2 susceptibility protein) c4(781) c5(782, 783, 784, 785, 786, 787) c6( ) c7(ng, atT, os, T, aC, avE, auW, aaH, e, bB, aeu, gf, ci, Jt, ip, gJ, aE, Ma, avA, ajv, avd, SV, Vv, ta, gs, K, hu, H, avq, xQ, zQ, aW, g, eF, bw, avx, aj, iP, nB, nF, U, cj, cX, avo, m, in, avB, iu, gU, ht, avy, n, aj, qN, br, Bl, avC, hw, TK, KN, eV, dL, dx, ia, avc, hT, aeU, agF, kp, avf, hq, YE, bP, dB, hk, Vy, hl, at, pg, avF, avr, it, B, L, bL, fM, avm, eX, ew, aA, bX, lu, AD, V, avD, q, J, er, eA, eJ, cF, afQ, BJ, eO, aT, avw, cy, vH, qv, bK, adV, ae, ne, jf, ak, iF, Uf, qw, ND, Y, aeS, r, aux, avh, eS, en, avb, cl, LV, t, ik, avz, ck, kP, Dy, Li, gX, wR, aan, fD, bt, ava, gV, pY, aZ, iW, jh, oZ, MC, Gn, ad, avu, dP, av, avp, hd, ib, db, yt, avs, bj, hP, aed, auY, BN, BO, Ub, hG, qt, Hh, ff, dq, Ej, aF, xH, KM, bz, dr, bF, es, Ew, bE, ag, auZ, X, afd, bC, be, aed, CW, jr, BA); #c1(BRCA2) c2(XP_011533505) c3(breast cancer type 2 susceptibility protein isoform X1) c4( ) c5(329596, 329597, 329598, 329599, 329600, 329601, 329602, 329603, 329604, 329605) c6( ) c7(ng, atT, os, T, aC, avE, auW, aaH, e, bB, aeu, gf, ci, Jt, ip, gJ, aE, Ma, avA, ajv, avd, SV, Vv, ta, gs, K, hu, H, avq, xQ, zQ, aW, g, eF, bw, avx, aj, iP, nB, nF, U, cj, cX, avo, m, in, avB, iu, gU, ht, avy, n, aj, qN, br, Bl, avC, hw, TK, KN, eV, dL, dx, ia, avc, hT, aeU, agF, kp, avf, hq, YE, bP, dB, hk, Vy, hL, at, pg, avF, avr, it, B, L, bL, fM, avm, eX, ew, aA, bX, lu, AD, V, avD, q, J, er, eA, eJ, cF, afQ, BJ, eO, aT, avw, cy, vH, qv, bK, adV, ae, ne, jf, ak, iF, Uf, qw, ND, Y, aeS, r, auX, avh, eS, en, avb, cl, LV, t, iK, avz, ck, kP, Dy, Li, gX, wR, aan, fD, bt, ava, gV, pY, aZ, iW, jh, oZ, MC, Gn, ad, avu, dP, av, avp, hd, ib, db, yt, avs, bj, hP, aeI, auY, BN, BO, Ub, hG, qt, Hh, ff, dq, Ej, aF, xH, KM, bz, dr, bF, es, Ew, bE, ag, auZ, X, afd, bC, be, aed, CW, jr, BA); #c1(BRCA2) c2(XP_011533507) c3(breast cancer type 2 susceptibility protein isoform X3) c4(329606) c5( ) c6( ) c7(ng, atT, os, T, aC, avE, auW, aaH, e, bB, aeu, gf, ci, Jt, ip, gJ, aE, Ma, avA, ajv, avd, SV, Vv, ta, gs, K, hu, H, avq, xQ, zQ, aW, g, eF, bw, avx, aj, iP, nB, nF, U, cj, cX, avo, m, in, avB, iu, gU, ht, avy, n, aj, qN, br, Bl, avC, hw, TK, KN, eV, dL, dx, ia, avc, hT, aeU, agF, kp, avf, hq, YE, bP, dB, hk, Vy, hL, at, pg, avF, avr, it, B, L, bL, fM, avm, eX, ew, aA, bX, Iu, AD, V, avD, q, J, er, eA, eJ, cF, afQ, BJ, eO, aT, avw, cy, vH, qv, bK, adV, ae, ne, jf, ak, iF, Uf, qw, ND, Y, aeS, r, aux, avh, eS, en, avb, cl, LV, t, iK, avz, ck, kP, Dy, Li, gX, wR, aan, fD, bt, ava, gV, pY, aZ, iW, jh, oZ, MC, Gn, ad, avu, dP, av, avp, hd, ib, db, yt, avs, bj, hP, aeI, auY, BN, BO, Ub, hG, qt, Hh, ff, dq, Ej, aF, xH, KM, bz, dr, bF, es, Ew, bE, ag, auZ, X, afd, bC, be, aed, CW, jr, BA); #c1(BRCC3) c2(NP_001018065) c3(lys-63-specific deubiquitinase BRCC36 isoform 2) c4(27031, 27032) c5(27033, 27034, 27035, 27036, 27037, 27038, 27039, 27040, 27041, 27042, 27043, 27044) c6( ) c7(ck, aW, Kk, qh, aT, atf, aS, bC, bd); #c1(BRCC3) c2(NP_001229569) c3(lys-63-specific deubiquitinase BRCC36 isoform 3) c4(84656) c5(84657, 84658, 84659, 84660, 84661, 84662) c6( ) c7(ck, aW, Kk, qh, aT, atf, aS, bC, bd); #c1(BRCC3) c2(NP_077308) c3(lys-63-specific deubiquitinase BRCC36 isoform 1) c4(215165, 215166) c5(215167, 215168, 215169, 215170, 215/71, 215172, 215173, 215174, 215175, 215176, 215177, 215/78) c6( ) c7(ck, aW, Kk, qh, aT, atf, aS, bC, bd); #c1(BRD1) c2(NP_001291737) c3(bromodomain-containing protein 1 isoform 1) c4(116459) c5(116460, 116461, 116462, 116463, 116464, 116465) c6( ) c7(ml, hS, mW, mZ, aq, qK, cz, mS, cd, mY, mT, K, ci, avG, he, mV, bK, uJ, B, ad, mX); #c1(BRD1) c2(NP_001291738) c3 (bromodomain-containing protein 1 isoform 2) c4(116466) c5(116467, 116468, 116469, 116470, 116471, 116472, 116473, 116474, 116475, 116476, 116477) c6( ) c7(mU, hS, mW, mZ, aq, qK, cz, mS, cd, mY, mT, K, ci, avG, he, mV, bK, uJ, B, ad, mX); #c1(BRD1) c2(XP_011528367) c3(bromodomain-containing protein 1 isoform X3) c4(323398) c5(323399, 323400, 323401, 323402, 323403, 323404) c6( ) c7(mU, hS, mW, mZ, aq, qK, cz, mS, cd, mY, mT, K, ci, avG, he, mV, bK, uJ, B, ad, mX); #c1(BRD1) c2(XP_011528372) c3(bromodomain-containing protein 1 isoform X7) c4( ) c5(323405, 323406, 323407, 323408, 323409, 323410) c6( ) c7(mU, hS, mW, mZ, aq, qK, cz, mS, cd, mY, mT, K, ci, avG, he, mV, bK, uJ, B, ad, mX); #c1(BRD1) c2(XP_011528373) c3(bromodomain-containing protein 1 isoform X2) c4( ) c5(323411) c6( ) c7(mU, hS, mW, mZ, aq, qK, cz, mS, cd, mY, mT, K, ci, avG, he, mV, bK, uJ, B, ad, mX); #c1(BRD1) c2(XP_011528375) c3(bromodomain-containing protein 1 isoform X9) c4(323412, 323413) c5(323414, 323415, 323416, 323417, 323418) c6( ) c7(mU, hS, mW, mZ, aq, qK, cz, mS, cd, mY, mT, K, ci, avG, he, mV, bK, uJ, B, ad, mX); #c1(BRD2) c2(NP_001106653) c3(bromodomain-containing protein 2 isoform 1) c4(42462, 42463, 42464, 42465, 42466, 42467, 42468, 42469) c5(42470, 42471, 42472, 42473, 42474, 42475, 42476, 42477, 42478, 42479, 42480, 42481, 42482, 42483, 42484, 42485, 42486, 42487, 42488, 42489, 42490, 42491, 42492, 42493, 42494, 42495, 42496, 42497, 42498, 42499, 42500, 42501, 42502, 42503, 42504, 42505, 42506, 42507, 42508, 42509, 42510, 42511, 42512, 42513, 42514, 42515, 42516, 42517, 42518, 42519, 42520, 42521, 42522, 42523, 42524, 42525, 42526, 42527, 42528, 42529, 42530, 42531, 42532, 42533, 42534, 42535, 42536, 42537, 42538, 42539, 42540, 42541, 42542, 42543, 42544, 42545, 42546, 42547, 42548, 42549, 42550, 42551, 42552, 42553, 42554, 42555, 42556, 42557, 42558, 42559, 42560, 42561, 42562, 42563, 42564, 42565, 42566, 42567, 42568, 42569, 42570, 42571, 42572, 42573) c6( ) c7(bM, T, Ut, UH, acz, amj, ci, Ul, il, qM, EL, eL, avM, yo, avH, aW, eM, aiM, m, Uw, Ro, OF, gU, Uc, lT, n, aj, nA, b, PD, adX, h, dL, hg, uV, avP, yq, zZ, avK, avN, bP, adZ, hL, Uv, hR, rx, zG, bL, fM, pQ, v, avO, q, J, BJ, aT, kf, vH, af, bA, kl, s, ND, aeS, r, avJ, lo, cl, pt, t, EJ, ck, pa, mf, UF, gx, Ke, aZ, iW, ha, jh, OE, iD, dG, ad, hX, dP, vJ, avL, Ps, avQ, yr, d, eZ, bz, YR, Cq, OG, Uu, avl, i); #c1(BRD2) c2(NP_001186384) c3(bromodomain-containing protein 2 isoform 2) c4(77520, 77521, 77522, 77523) c5(77524, 77525, 77526, 77527, 77528, 77529, 77530, 77531, 77532, 77533, 77534, 77535, 77536, 77537, 77538, 77539, 77540, 77541, 77542, 77543, 77544, 77545, 77546, 77547, 77548, 77549, 77550, 77551, 77552, 77553, 77554, 77555, 77556, 77557, 77558, 77559, 77560, 77561, 77562, 77563, 77564, 77565, 77566, 77567, 77568, 77569, 77570, 77571, 77572, 77573, 77574, 77575, 77576, 77577) c6( ) c7(bM, T, Ut, UH, acz, amj, ci, Ul, il, qM, EL, eL, avM, yo, avH, aW, eM, aiM, m, Uw, Ro, OF, gU, Uc, lT, n, aj, nA, b, PD, adX, h, dL, hg, uV, avP, yq, zZ, avK, avN, bP, adZ, hL, Uv, hR, rx, zG, bL, fM, pQ, v, avO, q, J, BJ, aT, kf, vH, af, bA, kl, s, ND, aeS, r, avJ, lo, cl, pt, t, EJ, ck, pa, mf, UF, gx, Ke, aZ, iW, ha, jh, DE, iD, dG, ad, hX, dP, vJ, avL, Ps, avl, yr, d, eZ, bz, YR, Cq, OG, Uu, avl, i); #c1(BRD2) c2(NP_001186385) c3(bromodomain-containing protein 2 isoform 3) c4(77578, 77579, 77580) c5(77581, 77582, 77583, 77584, 77585, 77586, 77587, 77588, 77589, 77590, 77591, 77592, 77593, 77594, 77595, 77596, 77597, 77598, 77599, 77600, 77601, 77602, 77603) c6( ) c7(bM, T, Ut, UH, acz, amj, ci, Ul, il, qM, EL, eL, avM, yo, avH, aW, eM, aiM, m, Uw, Ro, OF, gU, Uc, lT, n, aj, nA, b, PD, adX, h, dL, hg, uV, avP, yq, zZ, avK, avN, bP, adZ, hL, Uv, hR, rx, zG, bL, fM, pl, v, avO, q, J, BJ, aT, kf, vH, af, bA, kl, s, ND, aeS, r, avJ, lo, cl, pt, t, EJ, ck, pa, mf, UF, gx, Ke, aZ, iW, ha, jh, DE, iD, dG, ad, hX, dP, vJ, avL, Ps, avQ, yr, d, eZ, bz, YR, Cq, OG, Uu, avl, i); #c1(BRD2) c2(NP_001278915) c3(bromodomain-containing protein 2 isoform 4) c4(110916, 110917) c5(110918, 110919, 110920, 110921, 110922, 110923, 110924, 110925, 110926, 110927, 110928, 110929, 110930, 110931, 110932, 110933, 110934, 110935, 110936, 110937, 110938, 110939, 110940, 110941, 110942, 110943, 110944, 110945, 110946, 110947, 110948, 110949, 110950, (10951) c6( ) c7(bM, T, Ut, UH, acz, amj, ci, Ul, il, qM, EL, eL, avM, yo, avH, aW, eM, aiM, m, Uw, Ro, OF, gU, Uc, lT, n, aj, nA, b, PD, adX, h, dL, hg, uV, avP, yq, zZ, avK, avN, bP, adZ, hL, Uv, hR, rx, zG, bL, fM, pl, v, avO, q, J, BJ, aT, kf, vH, af, bA, kl, s, ND, aeS, r, avJ, lo, cl, pt, t, EJ, ck, pa, mf, UF, gx, Ke, aZ, iW, ha, jh, OE, iD, dG, ad, hX, dP, vJ, avL, Ps, avQ, yr, d, eZ, bz, YR, Cq, OG, Uu, avl, i); #c1(BRD3) c2(NP_031397) c3(bromodomain-containing protein 3) c4(172231, 172232, 172233) c5(172234, 172235, 172236, 172237, 172238, 172239, 172240, 172241, 172242, 172243, 172244, 172245, 172246, 172247, 172248, 172249, 172250, 172251) c6( ) c7(T, c); #c1(BRD3) c2(XP_005272271) c3(bromodomain-containing protein 3 isoform X2) c4(283951, 283952) c5(283953, 283954, 283955, 283956, 283957, 283958, 283959, 283960, 283961, 283962, 283963, 283964, 283965, 283966, 283967, 283968, 283969) c6( ) c7(T, c); #c1(BRD4) c2(XP_011526156) c3(bromodomain-containing protein 4 isoform X1) c4(320421) c5(320422, 320423, 320424, 320425, 320426, 320427, 320428, 320429) c6( ) c7(bM, T, Dw, Ke, jx, jh, fd, bB, ci, q, eW, F, afn, aT, h, hP, cp, jf, avR, Y, aW, r, tv, aq, tu, avT, ck, cT, rR, avS, bE, bt, avl, B); #c1(BRD4) c2(XP_011526157) c3(bromodomain-containing protein 4 isoform X3) c4(320430) c5(320431, 320432, 320433, 320434, 320435, 320436) c6( ) c7(bM, T, Dw, Ke, jx, jh, fd, bB, ci, q, eW, F, afn, aT, h, hP, cp, jf, avR, Y, aW, r, tv, aq, tu, avT, ck, cT, rR, avS, bE, bt, avU, B); #c1(BRD4) c2(XP_011526158) c3(bromodomain-containing protein 4 isoform X2) c4(320437) c5(320438, 320439, 320440, 320441, 320442, 320443) c6( ) c7(bM, T, Dw, Ke, jx, jh, fd, bB, ci, q, eW, F, afn, aT, h, hP, cp, jf, avR, Y, aW, r, tv, aq, tu, avT, ck, cl, rR, avS, bE, bt, avU, B); #c1(BRD7) c2(NP_001167455) c3(bromodomain-containing protein 7 isoform 1) c4(68934) c5(68935, 68936, 68937, 68938, 68939, 68940, 68941, 68942, 68943) c6( ) c7(T, kP, eH, fe, ci, m, k, eF, t, bt, eA, c1); #c1(BRD7) c2(NP_037395) c3(bromodomain-containing protein 7 isoform 2) c4(175244) c5(175245, 175246, 175247, 175248, 175249, 175250, (75251) c6( ) c7(T, kP, eH, fe, ci, m, k, eF, t, bt, eA, c1); #c1(BRD8) c2(NP_001157798) c3(bromodomain-containing protein 8 isoform 4) c4(62706, 62707) c5(62708, 62709, 62710, 62711, 62712, 62713, 62714, 62715, 62716, 62717, 62718, 62719) c6( ) c7(dP, T, aE, af, qt, aW, X, gd, aT, r, ci, eA, zy, AD, q); #c1(BRD8) c2(NP_001287891) c3(bromodomain-containing protein 8 isoform 6) c4(113143) c5(113144, 113145, 113146, 113147, 113148, 113149) c6( ) c7(dP, T, aE, af, qt, aW, X, gd, aT, r, ci, eA, zy, AO, q); #c1(BRD8) c2(NP_001287895) c3(bromodomain-containing protein & isoform 7) c4(113178) c5( ) c6( ) c7(dP, T, aE, af, qt, aW, X, gd, aT, r, ci, eA, zy, AD, q); #c1(BRD8) c2(NP_006687) c3(bromodomain-containing protein 8 isoform 1) c4(167157) c5(167158, 167159, 167160, 167161, 167162, 167163) c6( ) c7(dP, T, aE, af, qt, aW, X, gd, aT, r, ci, eA, zy, AO, q); #c1(BRD8) c2(NP_631938) c3(bromodomain-containing protein 8 isoform 2) c4(238356) c5(238357, 238358, 238359, 238360, 238361, 238362) c6( ) c7(dP, T, aE, af, qt, aW, X, gd, aT, r, ci, eA, zy, AO, q); #c1(BRD8) c2(XP_006714588) c3(bromodomain-containing protein 8 isoform X9) c4(287914) c5(287915, 287916, 287917, 287918, 287919, 287920, 287921, 287922, 287923, 287924, 287925, 287926) c6( ) c7(dP, T, aE, af, qt, aW, X, gd, aT, r, ci, eA, zy, AD, q); #c1(BRE) c2(NP_004890) c3(BRCA1-A complex subunit BRE isoform 1) c4(150594) c5(150595, 150596, 150597, 150598, 150599, 150600) c6( ) c7(DA, iZ, pN, aW, aT, ci, bt, B); #c1(BRE) c2(NP_954662) c3(BRCA1-A complex subunit BRE isoform 3) c4(261197, 261198) c5(261199, 261200, 26/201, 261202, 261203, 261204, 261205, 261206, 261207, 261208, 261209, 261210, 261211, 261212) c6( ) c7(DA, iZ, pN, aW, aT, ci, bt, B); #c1(BRE) c2(NP_954664) c3(BRCA1-A complex subunit BRE isoform 2) c4(261213, 261214) c5(261215, 261216, 261217, 261218, 261219, 261220, 261221, 261222, 261223, 261224, 261225, 261226, 261227, 261228, 261229, 261230, 261231, 261232) c6( ) c7(DA, iZ, pN, aW, aT, ci, bt, B); #c1(BRF1) c2(NP_001229715) c3 (transcription factor IIIB 90 kDa subunit isoform 4) c4(84863) c5(84864, 84865, 84866, 84867, 84868) c6( ) c7(ae, ad); #c1(BRF1) c2(NP_001229716) c3(transcription factor IIIB 90 kDa subunit isoform 5) c4(84869) c5( ) c6( ) c7(ae, ad); #c1(BRF1) c2(NP_001229717) c3(transcription factor IIIB 90 kDa subunit isoform 6) c4(84870) c5(84871, 84872, 84873, 84874, 84875, 84876) c6( ) c7(ae, ad); #c1(BRF1) c2(NP_001229718) c3(transcription factor IIIB 90 kDa subunit isoform 7) c4( ) c5(84877, 84878, 84879, 84880, 84881, 84882, 84883, 84884, 84885, 84886, 84887) c6( ) c7(ae, ad); #c1(BRF1) c2(NP_001229719) c3(transcription factor IIIB 90 kDa subunit isoform 8) c4(84888, 84889) c5(84890, 84891, 84892, 84893, 84894, 84895, 84896, 84897, 84898, 84899, 84900, 84901) c6( ) c7(ae, ad); #c1(BRF1) c2(NP_001510) c3(transcription factor IIIB 90 kDa subunit isoform 1) c4(118386, (18387) c5(118388, 118389, 118390, 118391, 118392, 118393, 118394, 118395, 118396, 118397, 118398, 118399, 118400, 118401, 118402, 118403, 118404, 118405, 118406) c6( ) c7(ae, ad); #c1(BRF1) c2(XP_005267618) c3 (transcription factor IIIB 90 kDa subunit isoform X2) c4(281100, 281101) c5( ) c6( ) c7(ae, ad); #c1(BRF1) c2(XP_005267620) c3(transcription factor IIIB 90 kDa subunit isoform X1) c4(281102, 281103, 281104, 281105) c5(281106, 281107, 281108, 281109, 281110, 281111, 281112, 281113, 281114, 281115, 281116, 281117, 281118, 281119, 281120, 281121, 281122, 281123, 281124, 281125, 281126, 281127, 281128, 281129, 281130, 281131, 281132, 281133, 281134, 281135) c6( ) c7(ae, ad); #c1(BRF1) c2(XP_006720186) c3(transcription factor IIIB 90 kDa subunit isoform X6) c4( ) c5(293309) c6( ) c7(ae, ad); #c1(BRF1) c2(XP_011534974) c3(transcription factor IIIB 90 kDa subunit isoform X3) c4( ) c5(331532) c6( ) c7(ae, ad); #c1(BRF1) c2(XP_011534975) c3(transcription factor IIIB 90 kDa subunit isoform X4) c4(331533, 331534) c5( ) c6( ) c7(ae, ad); #c1(BRF1) c2(XP_011534976) c3(transcription factor IIIB 90 kDa subunit isoform X5) c4( ) c5(331535, 331536, 331537, 331538, 331539, 331540) c6( ) c7(ae, ad); #c1(BRF2) c2(NP_060780) c3(transcription factor (IIB 50 kDa subunit) c4(197747, 197748) c5(197749, 197750, 197751, 197752, 197753, 197754, 197755, 197756, 197757, 197758, 197759, 197760) c6( ) c7(Qu, T, eM, ff, en); #c1(BRI3) c2(NP_001152963) c3(brain protein 13 isoform b) c4(60285) c5(60286, 60287, 60288, 60289, 60290, 60291) c6( ) c7(bL, Qu, T, Qj, Tb, QO, if, avX, aA, bB, n, aeu, Gn, ci, bh, qN, q, ad, eA, av, iu, wG, awa, Xe, awb, pX, aT, kf, h, Ou, dL, fb, nJ, ae, af, bc, eL, jf, iF, A, avV, d, aW, Y, sq, kK, FD, ff, r, qL, YG, aF, bp, en, cl, g, t, f, bz, pb, avW, bN, as, mK, nM, kP, Dy, eM, dB, se, bE, go, JA, mx, hL, Om, avZ, AM, m, act, hh, rA, it, YK, avY); #c1(BRI3) c2(NP_056194) c3(brain protein 13 isoform a) c4(186253) c5(186254, 186255, 186256, 186257, 186258, 186259) c6( ) c7(bL, Qu, T, Qj, Tb, QO, if, avX, aA, bB, n, aeu, Gn, ci, bh, qN, q, ad, eA, av, iu, wG, awa, Xe, awb, pX, aT, kf, h, Ou, dL, fb, nJ, ae, af, bc, eL, jf, iF, A, avV, d, aW, Y, sq, KK, FD, ff, r, qL, YG, aF, bp, en, cl, g, t, f, bz, pb, avW, bN, as, mK, nM, kP, Dy, eM, dB, se, bE, go, JA, mx, hL, Om, avZ, AM, m, act, hh, rA, it, YK, avY); #c1(BRI3BP) c2(NP_542193) c3(BRI3-binding protein precursor) c4(232266) c5(232267, 232268, 232269, 232270, 232271, 232272) c6( ) c7(ck, T, aW, if, r, aT, ci, qN, q); #c1(BRINP1) c2(NP_055433) c3(BMP/retinoic acid-inducible neural-specific protein 1 precursor) c4(181106, 181107) c5(181108, 181109, 181110, 181111, 181112, 181113, 181114, 181115, 181116, 181117, 181118, 181119, 181120, 181121, 181122, 181123) c6( ) c7(x, bM, T, ant, id, eM, aW, aw, ff, jh, aT, la, n, dL, Da, aF, en, an, V, eL, cd); #c1(BRINP2) c2(NP_066988) c3(BMP/retinoic acid-inducible neural-specific protein 2 precursor) c4(208043, 208044) c5(208045, 208046, 208047, 208048, 208049, 208050, 208051, 208052, 208053, 208054, 208055, 208056, 208057, 208058, 208059, 208060) c6(208061, 208062, 208063, 208064) c7(oe, fq); #c1(BRINP3) c2(NP_950252) c3(BMP/retinoic acid-inducible neural-specific protein 3 precursor) c4(260938, 260939) c5(260940, 260941, 260942, 260943, 260944, 260945, 260946, 260947, 260948, 260949, 260950, 260951, 260952, 260953, 260954) c6(260955, 260956, 260957) c7(ba, dD, ac, aw, bg, EQ, fe, j, eq, Tz); #c1(BRIP1) c2(NP_114432) c3(Fanconi anemia group J protein) c4(222760) c5(222761, 222762, 222763, 222764, 222765, 222766) c6( ) c7(T, os, aC, yk, if, n, oZ, ci, qN, q, ad, eA, ip, F, Dk, awd, oW, aT, h, aed, dL, ji, K, ae, sb, aW, CT, r, aq, g, zE, t, bz, dr, dB, dc, afd, at, m, bt, B, awc); #c1(BRK1) c2(NP_060932) c3(protein BRICK1) c4(198756, 198757) c5(198758, 198759, 198760, 198761, 198762, 198763, 198764, 198765, 198766, 198767, 198768, 198769) c6( ) c7(Jm, es, T, py, RI, auo, awe, Qu, By, Hu, fr, K, Qz, bz, VB); #c1(BRMS1) c2(NP_001020128) c3(breast cancer metastasis-suppressor 1 isoform 2) c4(28267) c5(28268, 28269, 28270, 28271, 28272, 28273, 28274) c6( ) c7(T, ci, eA, av, aT, dL, Dz, nJ, eL, B, CN, aW, Y, ff, en, g, zE, t, bz, Dy, eM, m, iu); #c1(BRMS1) c2(NP_056214) c3(breast cancer metastasis-suppressor 1 isoform 1) c4(186443, 186444) c5(186445, 186446, 186447, 186448, 186449, 186450, 186451, 186452, 186453, 186454, 186455) c6( ) c7(T, ci, eA, av, aT, dL, Dz, nJ, eL, B, CN, aW, Y, ff, en, g, zE, t, bz, Dy, eM, m, iu); #c1(BRMS1L) c2(NP_115728) c3(breast cancer metastasis-suppressor 1-like protein) c4(223722, 223723) c5(223724, 223725, 223726, 223727, 223728, 223729, 223730) c6( ) c7(T, aw, ci, Ea, eA, av, iu, aT, dL, Dz, nJ, eL, af, CN, aW, Y, ff, en, g, zE, t, bz, Dy, eM, nB, cj, m, B); #c1(BRMS1L) c2(XP_006720338) c3(breast cancer metastasis-suppressor 1-like protein isoform X3) c4( ) c5(293497, 293498, 293499, 293500, 293501) c6( ) c7(T, aw, ci, Ea, eA, av, iu, aT, dL, Dz, nJ, eL, af, CN, aW, Y, ff, en, g, zE, t, bz, Dy, eM, nB, cj, m, B); #c1(BRS3) c2(NP_001718) c3(bombesin receptor subtype-3) c4(120471) c5(120472, 120473, 120474, 120475, 120476, 120477) c6( ) c7(bL, bM, T, iH, yk, hU, ha, cz, fd, aj, v, ci, yp, ye, xY, J, cd, ad, eA, yn, av, xW, pX, aT, h, xV, bK, K, yb, yh, yj, ae, yi, xX, d, xU, aW, Y, yc, yq, t, nC, bz, ik, iP, dR, jq, ok, yf, bP, yl, xZ, cj, tl, m, y); #c1(BRSK1) c2(NP_115806) c3(serine/threonine-protein kinase BRSK1) c4(223971, 223972) c5(223973, 223974, 223975, 223976, 223977, 223978) c6( ) c7(il, aW, zZ, gv, aT); #c1(BRSK1) c2(XP_005259384) c3(serine/threonine-protein kinase BRSK1 isoform X2) c4( ) c5(276083, 276084, 276085, 276086, 276087, 276088, 276089, 276090, 276091, 276092) c6( ) c7(il, aW, zZ, gv, aT); #c1(BRSK2) c2(NP_001243556) c3(serine/threonine-protein kinase BRSK2 isoform 1) c4(91039) c5(91040, 91041, 91042, 91043, 91044, 91045) c6( ) c7(ft); #c1(BRSK2) c2(NP_001243558) c3(serine/threonine-protein kinase BRSK2 isoform 3) c4(91046, 91047) c5(91048, 91049, 91050, 91051, 91052, 91053, 91054, 91055, 91056, 91057, 91058, 91059, 91060, 91061, 91062, 91063, 91064) c6( ) c7(ft); #c1(BRSK2) c2(NP_001243559) c3(serine/threonine-protein kinase BRSK2 isoform 4) c4(91065) c5(91066, 91067, 91068, 91069, 91070, 91071) c6( ) c7(ft); #c1(BRSK2) c2(NP_001269147) c3(serine/threonine-protein kinase BRSK2 isoform 5) c4( ) c5(100708, 100709, 100710, 100711, 100712) c6( ) c7(ft); #c1(BRSK2) c2(NP_003948) c3(serine/threonine-protein kinase BRSK2 isoform 2) c4(142391) c5(142392, 142393, 142394, 142395, 142396, 142397) c6( ) c7(ft); #c1(BRSK2) c2(XP_005253277) c3(serine/threonine-protein kinase BRSK2 isoform X9) c4( ) c5(272366) c6(272367) c7(ft); #c1(BRSK2) c2(XP_006718437) c3(serine/threonine-protein kinase BRSK2 isoform X2) c4(291873) c5( ) c6( ) c7(ft); #c1(BRWD1) c2(NP_001007247) c3(bromodomain and WD repeat-containing protein 1 isoform C) c4(22643) c5(22644, 22645, 22646, 22647, 22648, 22649) c6( ) c7(fe, f, bK, ND, iV); #c1(BRWD1) c2(NP_061836) c3(bromodomain and WD repeat-containing protein 1 isoform A) c4(200702) c5(200703, 200704, 200705, 200706, 200707, 200708) c6( ) c7(fe, f, bK, ND, iV); #c1(BRWD1) c2(NP_387505) c3(bromodomain and WD repeat-containing protein 1 isoform B) c4(229006) c5(229007, 229008, 229009, 229010, 229011, 229012) c6( ) c7(fe, f, bK, ND, iV); #c1(BRWD1) c2(XP_011527913) c3(bromodomain and WD repeat-containing protein 1 isoform X1) c4(323010) c5(323011, 323012, 323013, 323014, 323015, 323016) c6( ) c7(fe, f, bK, ND, iV); #c1(BRWD3) c2(NP_694984) c3(bromodomain and WD repeat-containing protein 3) c4(245555) c5(245556, 245557, 245558, 245559, 245560, 245561) c6( ) c7(nf, awf); #c1(BRWD3) c2(XP_011529206) c3(bromodomain and WD repeat-containing protein 3 isoform X3) c4( ) c5(324623) c6( ) c7(nf, awf); #c1(BSCL2) c2(NP_001116427) c3(seipin isoform 1) c4(43818, 43819, 43820, 43821, 43822, 43823, 43824, 43825) c5(43826, 43827, 43828, 43829, 43830, 43831, 43832, 43833, 43834, 43835, 43836, 43837, 43838, 43839, 43840, 43841, 43842, 43843, 43844, 43845, 43846, 43847, 43848, 43849, 43850, 43851, 43852, 43853, 43854, 43855, 43856, 43857, 43858, 43859, 43860, 43861, 43862, 43863, 43864, 43865, 43866, 43867, 43868, 43869) c6( ) c7(Pt, Ol, aj, fu, nA, awg, J, LL, LN, lu, kf, jy, vc, bl, K, bU, bV, iU, LO, fc, as, LK, GR, dr, JP, LM, c, cP); #c1(BSCL2) c2(NP_001124/74) c3(seipin isoform 3) c4(48417, 48418) c5(48419, 48420, 48421, 48422, 48423, 48424, 48425, 48426, 48427, 48428, 48429, 48430, 48431, 48432, 48433, 48434) c6( ) c7(Pt, Ol, aj, fu, nA, awg, J, LL, LN, lu, kf, jy, vc, bl, K, bU, bV, iU, LO, fc, as, LK, GR, dr, JP, LM, c, cP); #c1(BSCL2) c2(NP_116056) c3(seipin isoform 2) c4( ) c5(225300, 225301, 225302, 225303, 225304, 225305, 225306, 225307, 225308, 225309, 225310) c6( ) c7(Pt, Ol, aj, fu, nA, awg, J, LL, LN, lu, kf, jy, vc, bl, K, bU, bV, iU, LO, fc, as, LK, GR, dr, JP, LM, c, cP); #c1(BSG) c2(NP_001719) c3(basigin isoform 1 precursor) c4(120478) c5(120479, 120480, 120481, 120482, 120483, 120484) c6( ) c7(T, ea, if, Xi, fd, bB, ci, ip, aE, K, hu, eL, hm, yo, aW, zC, g, as, eF, cm, qJ, ba, nB, vA, m, dD, ht, n, fr, qN, PJ, ai, ft, IG, eV, dL, aq, W, dB, aar, uz, it, AN, B, cn, vQ, AS, lH, eX, aA, awh, q, eA, eJ, yw, qK, aT, cy, ae, af, s, oV, Y, bg, en, t, Px, Vi, iK, ck, ov, zu, GI, id, eq, ad, dP, yt, fb, d, ff, eH, cr, eZ, aF, anW, bz, bE, ll, gZ, lC, y); #c1(BSG) c2(NP_940992) c3(basigin isoform 3) c4( ) c5(260274, 260275, 260276, 260277, 260278) c6( ) c7(T, ea, if, Xi, fd, bB, ci, ip, aE, K, hu, eL, hm, yo, aW, zC, g, as, eF, cm, qJ, ba, nB, vA, m, dD, ht, n, fr, qN, PJ, ai, ft, lG, eV, dL, aq, W, dB, aar, uz, it, AN, B, cn, vQ, AS, lH, eX, aA, awh, q, eA, eJ, yw, qK, aT, cy, ae, af, s, oV, Y, bg, en, t, Px, Vi, iK, ck, ov, zu, Gl, id, eq, ad, dP, yt, fb, d, ff, eH, cr, eZ, aF, anW, bz, bE, ll, gZ, lC, y); #c1(BSG) c2(NP_940993) c3(basigin isoform 4) c4(260279) c5(260280, 260281, 260282, 260283, 260284, 260285) c6( ) c7(T, ea, if, Xi, fd, bB, ci, ip, aE, K, hu, eL, hm, yo, aW, zC, g, as, eF, cm, qJ, ba, nB, vA, m, dD, ht, n, fr, qN, PJ, ai, ft, lG, eV, dL, aq, W, dB, aar, uz, it, AN, B, cn, vQ, AS, lH, eX, aA, awh, q, eA, eJ, yw, qK, aT, cy, ae, af, s, oV, Y, bg, en, t, Px, Vi, iK, ck, ov, zu, Gl, id, eq, ad, dP, yt, fb, d, ff, eH, cr, eZ, aF, anW, bz, bE, ll, gZ, lC, y); #c1(BSG) c2(XP_005259676) c3(basigin isoform X1) c4(276222, 276223) c5(276224, 276225, 276226, 276227, 276228, 276229, 276230) c6( ) c7(T, ea, if, Xi, fd, bB, ci, ip, aE, K, hu, eL, hm, yo, aW, zC, g, as, eF, cm, qJ, ba, nB, vA, m, dD, ht, n, fr, qN, PJ, ai, ft, lG, eV, dL, aq, W, dB, aar, uz, it, AN, B, cn, vQ, AS, lH, eX, aA, awh, q, eA, eJ, yw, qK, aT, cy, ae, af, s, oV, Y, bg, en, t, Px, Vi, iK, ck, ov, zu, Gl, id, eq, ad, dP, yt, fb, d, ff, eH, cr, eZ, aF, anW, bz, bE, ll, gZ, lC, y); #c1(BSN) c2(NP_003449) c3(protein bassoon) c4(138430) c5(138431, 138432, 138433, 138434, 138435, 138436, 138437, 138438) c6( ) c7(bL, A, U, cz, gZ, GC, fe, j, bK, iV, y, V, xb); #c1(BSPH1) c2(NP_001121798) c3(binder of sperm protein homolog 1 precursor) c4(46251) c5(46252, 46253, 46254, 46255, 46256, 46257) c6( ) c7(iU, bt, iT); #c1(BSPRY) c2(NP_060158) c3(B box and SPRY domain-containing protein) c4(194525) c5(194526, 194527, 194528, 194529, 194530, 194531) c6( ) c7(Na, Nc); #c1(BST1) c2(NP_004325) c3(ADP-ribosyl cyclase/cyclic ADP-ribose hydrolase 2 precursor) c4(145184) c5(145185, 145186, 145187, 145188, 145189, 145190) c6( ) c7(bM, m, K, an, y, bN, aa); #c1(BST2) c2(NP_004326) c3(bone marrow stromal antigen 2 precursor) c4(145191, 145192) c5(145193, 145194, 145195, 145196, 145197, 145198, 145199, 145200, 145201, 145202, 145203, 145204) c6( ) c7(ck, T, dB, aW, Y, AH, hL, aT, h, fd, fr, gn, m, cl, f, t, as, awi); #c1(BSX) c2(NP_001091639) c3(brain-specific homeobox protein homolog) c4(38525) c5(38526, 38527, 38528, 38529, 38530, 38531) c6( ) c7(v); #c1(BTBD1) c2(NP_001011885) c3(BTB/POZ domain-containing protein 1 isoform 2) c4(24807) c5(24808, 24809, 24810, 24811, 24812, 24813) c6( ) c7(If, awj); #c1(BTBD1) c2(NP_079514) c3(BTB/POZ domain-containing protein 1 isoform 1) c4(218502) c5(218503, 218504, 218505, 218506, 218507, 218508, 218509) c6( ) c7(If, awj); #c1(BTBD10) c2(XP_011518706) c3(BTB/POZ domain-containing protein 10 isoform X1) c4(313105, 313106, 313107) c5(313108, 313109, 313110, 313111, 313112, 313113, 313114, 313115, 313116, 313117, 313118, 313119, 313120, 313121, 313122, 313123, 313124, 313125) c6( ) c7(c); #c1(BTBD11) c2(NP_001017523) c3(ankyrin repeat and BTB/POZ domain-containing protein BTBD11 isoform b) c4(26546) c5(26547, 26548, 26549, 26550, 26551, 26552, 26553, 26554, 26555, 26556, 26557, 26558) c6( ) c7(aq, aw, c); #c1(BTBD11) c2(NP_001018082) c3(ankyrin repeat and BTB/POZ domain-containing protein BTBD11 isoform a) c4(27147, 27148, 27149) c5(27150, 27151, 27152, 27153, 27154, 27155, 27156, 27157, 27158, 27159, 27160, 27161) c6( ) c7(aq, aw, c); #c1(BTBD11) c2(XP_011536210) c3(ankyrin repeat and BTB/POZ domain-containing protein BTBD11 isoform X1) c4(333520) c5(333521, 333522, 333523, 333524, 333525, 333526) c6( ) c7(aq, aw, c); #c1(BTBD16) c2(NP_653188) c3(BTB/POZ domain-containing protein 16) c4(238986) c5(238987, 238988, 238989, 238990, 238991, 238992) c6( ) c7(bK, W); #c1(BTBD2) c2(NP_060267) c3(BTB/POZ domain-containing protein 2) c4(195138) c5(195139, 195140, 195141, 195142, 195143, 195144, 195145) c6( ) c7(dB, eA, W); #c1(BTBD3) c2(NP_001269480) c3(BTB/POZ domain-containing protein 3 isoform b) c4( ) c5(101735, 101736, 101737, 101738, 101739) c6( ) c7(cd, j, ac); #c1(BTBD3) c2(NP_055777) c3(BTB/POZ domain-containing protein 3 isoform a) c4(183432, 183433) c5(183434, 183435, 183436, 183437, 183438, 183439, 183440, 183441, 183442, 183443, 183444, 183445, 183446, 183447, 183448, 183449, 183450, 183451, 183452, 183453, 183454, 183455) c6( ) c7(cd, j, ac); #c1(BTBD3) c2(NP_852108) c3(BTB/POZ domain-containing protein 3 isoform d) c4(254851) c5(254852) c6( ) c7(cd, j, ac); #c1(BTBD9) c2(NP_001165889) c3(BTB/POZ domain-containing protein 9 isoform c) c4(67937) c5(67938, 67939, 67940, 67941, 67942, 67943) c6( ) c7(eR, Fo, yL, yY, awl, awk, aY, awm, qc); #c1(BTBD9) c2(NP_689946) c3(BTB/POZ domain-containing protein 9 isoform b) c4(244449) c5( ) c6( ) c7(eR, Fo, yL, yY, awl, awk, aY, awm, qc); #c1(BTBD9) c2(XP_005248898) c3(BTB/POZ domain-containing protein 9 isoform X1) c4(268754, 268755) c5(268756, 268757, 268758, 268759, 268760, 268761, 268762, 268763, 268764, 268765, 268766, 268767, 268768, 268769, 268770, 268771, 268772, 268773, 268774, 268775) c6( ) c7(eR, Fo, yL, yY, aw, awk, aY, awm, qc); #c1(BTBD9) c2(XP_011512584) c3(BTB/POZ domain-containing protein 9 isoform X4) c4( ) c5(303495, 303496, 303497, 303498, 303499, 303500) c6( ) c7(eR, Fo, yL, yY, awl, awk, aY, awm, qc); #c1(BTC) c2(NP_001720) c3(probetacellulin precursor) c4(120485) c5(120486, 120487, 120488, 120489, 120490, 120491, 120492, 120493) c6(120494, 120495) c7(bL, T, mj, A, hV, aW, aT, zo, aj, mf, dG, im, nE, AO, B, J, auD); #c1(BTD) c2(NP_000051) c3(biotinidase isoform 3) c4(788) c5(789, 790, 791, 792, 793, 794, 795) c6( ) c7(bN, cZ, b, T, PK, Cn, awn, aW, oe, Bw, i, lZ, aT, zN, awo, j, dd, ex, mH, awp); #c1(BTD) c2(NP_001268652) c3(biotinidase isoform 1) c4(100071) c5(100072, 100073, 100074, 100075, 100076, 100077, 100078, 100079, 100080, 100081, 100082) c6( ) c7(bN, cZ, b, T, PK, Cn, awn, aW, oe, Bw, i, lZ, aT, zN, awo, j, dd, ex, mH, awp); #c1(BTD) c2(NP_001268653) c3(biotinidase isoform 2) c4(100083, 100084) c5(100085, 100086, 100087, 100088, 100089, 100090) c6( ) c7(bN, cZ, b, T, PK, Cn, awn, aW, oe, Bw, i, lZ, aT, zN, awo, j, dd, ex, mH, awp); #c1(BTF3) c2(NP_001032726) c3(transcription factor BTF3 isoform A) c4(31440) c5(31441, 31442, 31443, 31444, 31445, 31446) c6( ) c7(T, d, aE, r, kp, g, q); #c1(BTF3) c2(NP_001198) c3(transcription factor BTF3 isoform B) c4(83520) c5(83521, 83522, 83523, 83524, 83525, 83526, 83527) c6( ) c7(T, d, aE, r, kp, g, q); #c1(BTG1) c2(NP_001722) c3(protein BTG1) c4(120513) c5(120514, 120515, 120516, 120517, 120518, 120519) c6( ) c7(ck, ae, T, x, eM, aW, bt, cj, aT, hL, fd, k, ajo, cl, jj, f, cM, V, awq, ad); #c1(BTG2) c2(NP_006754) c3(protein BTG2) c4(167731) c5(167732, 167733, 167734, 167735, 167736, 167737) c6( ) c7(T, awr, ci, ad, py, Bc, aT, lG, dL, K, ae, eL, eu, li, jf, aW, EU, cm, bz, es, iA, NI, aS, aws, y, rA, B); #c1(BTG3) c2(NP_001124386) c3(protein BTG3 isoform a) c4(48769, 48770) c5(48771, 48772, 48773, 48774, 48775, 48776) c6( ) c7(ae, T, es, B, av, iu, iA, lu, y, eZ, oX, m, fl, pb, fr, t, bz, mC, ad, bc, hq); #c1(BTG3) c2(NP_006797) c3 (protein BTG3 isoform b) c4(168161) c5(168162, 168163, 168164, 168165, 168166, 168167, 168168, 168169, 168170, 168171, 168172) c6( ) c7(ae, T, es, B, av, iu, iA, lu, y, eZ, oX, m, fl, pb, fr, t, bz, mC, ad, bc, hq); #c1 (BTG3) c2(XP_011527743) c3(protein BTG3 isoform X1) c4( ) c5(322726) c6( ) c7(ae, T, es, B, av, iu, iA, lu, y, eZ, oX, m, fl, pb, fr, t, bz, mC, ad, bc, hq); #c1 (BTG4) c2(NP_060059) c3(protein BTG4) c4(193924) c5(193925, 193926, 193927, 193928, 193929, 193930, 193931, 193932, 193933, 193934, 193935, 193936) c6( ) c7(yg, r, cj, aq, q, B); #c1(BTG4) c2(XP_011541181) c3(protein BTG4 isoform X4) c4(339786) c5( ) c6( ) c7(yg, r, cj, aq, q, B); #c1(BTK) c2(NP_000052) c3(tyrosine-protein kinase BTK isoform 1) c4(796, 797, 798, 799, 800, 801) c5(802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835) c6( ) c7(gV, T, fM, dA, ajm, fF, fd, eW, zn, h, zA, ey, Wm, amA, d, hG, Cw, oV, Rj, Of, aw, awu, Og, bw, aww, Wt, asE, ck, eM, awt, cj, hO, k, bt, Ze, afX); #c1 (BTK) c2(NP_001274273) c3(tyrosine-protein kinase BTK isoform 3) c4(106196) c5(106197, 106198, 106199, 106200, 106201, 106202, 106203) c6( ) c7(gV, T, fM, dA, ajm, fF, fd, eW, zn, h, zA, ey, Wm, amA, d, hG, Cw, oV, Rj, Of, aw, awu, Og, bw, aww, Wt, asE, ck, eM, awt, cj, hO, k, bt, Ze, afX); #c1(BTK) c2(NP_001274274) c3(tyrosine-protein kinase BTK isoform 2) c4(106204, 106205) c5(106206, 106207, 106208, 106209, 106210, 106211, 106212, 106213, 106214, 106215, 106216, 106217, 106218) c6( ) c7(gV, T, fM, dA, ajm, fF, fd, eW, zn, h, zA, ey, Wm, amA, d, hG, Cw, oV, Rj, Of, aw, awu, Og, bw, aww, Wt, asE, ck, eM, awt, cj, hO, k, bt, Ze, afX); #c1(BTLA) c2(NP_001078826) c3(B- and T-lymphocyte attenuator isoform 2) c4(37912) c5(37913, 37914, 37915, 37916, 37917, 37918) c6( ) c7(qw, A, fM, xE, qZ, aW, jx, eZ, cj, aT, h, jh, y, eW); #c1 (BTLA) c2(NP_861445) c3(B- and T-lymphocyte attenuator isoform 1 precursor) c4(255580) c5(255581, 255582, 255583, 255584, 255585, 255586) c6( ) c7(qw, A, fM, xE, qZ, aW, jx, eZ, cj, aT, h, jh, y, eW); #c1(BTN1A1) c2(NP_001723) c3(butyrophilin subfamily 1 member A1 precursor) c4( ) c5(120520, 120521, 120522, 120523, 120524, 120525, 120526, 120527, 120528, 120529) c6( ) c7(y, nM, K, U); #c1(BTN1A1) c2(XP_005249397) c3(butyrophilin subfamily 1 member A1 isoform X1) c4(269293, 269294) c5(269295, 269296) c6( ) c7(y, nM, K, U); #c1 (BTN2A1) c2(NP_001184162) c3(butyrophilin subfamily 2 member A1 isoform 3) c4( ) c5(75437, 75438, 75439, 75440, 75441) c6( ) c7(bL, aw, eZ, cz, aj, pl, ei, J); #c1(BTN2A1) c2(NP_001184163) c3(butyrophilin subfamily 2 member A1 isoform 4 precursor) c4(75442) c5(75443, 75444, 75445, 75446, 75447, 75448) c6( ) c7(bL, aw, eZ, cz, aj, pl, ei, J); #c1(BTN2A1) c2(NP_008980) c3(butyrophilin subfamily 2 member A1 isoform 1 precursor) c4(170017, 170018, 170019) c5(170020, 170021, 170022, 170023, 170024, 170025, 170026, 170027, 170028, 170029, 170030, 170031, 170032) c6( ) c7(bL, aw, eZ, cz, aj, pl, ei, J); #c1(BTN2A1) c2(NP_510961) c3(butyrophilin subfamily 2 member A1 isoform 2 precursor) c4(231076) c5(231077, 231078, 231079, 231080, 231081, 231082) c6( ) c7(bL, aw, eZ, cz, aj, pl, ei, J); #c1(BTN2A2) c2(NP_001184166) c3(butyrophilin subfamily 2 member A2 isoform a precursor) c4(75449) c5(75450, 75451, 75452, 75453, 75454, 75455, 75456, 75457, 75458, 75459) c6( ) c7(CT); #c1(BTN2A2) c2(NP_001184167) c3(butyrophilin subfamily 2 member A2 isoform c precursor) c4(75460, 75461, 75462) c5(75463, 75464, 75465, 75466, 75467, 75468, 75469, 75470, 75471, 75472, 75473, 75474, 75475, 75476, 75477, 75478, 75479, 75480, 75481, 75482, 75483, 75484, 75485, 75486) c6( ) c7(CT); #c1(BTN2A2) c2(NP_001184168) c3(butyrophilin subfamily 2 member A2 isoform d precursor) c4(75487) c5(75488, 75489, 75490, 75491, 75492, 75493) c6( ) c7(CT); #c1(BTN2A2) c2(NP_001184169) c3(butyrophilin subfamily 2 member AZ isoform e precursor) c4(75494) c5(75495, 75496, 75497, 75498, 75499, 75500, 75501, 75502, 75503, 75504, 75505) c6( ) c7(CT); #c1(BTN2A2) c2(NP_853509) c3(butyrophilin subfamily 2 member A2 isoform b precursor) c4(255132, 255133, 255134) c5( ) c6( ) c7(CT); #c1(BTN2A2) c2(XP_006715017) c3(butyrophilin subfamily 2 member A2 isoform X5) c4( ) c5(288515, 288516, 288517, 288518, 288519, 288520) c6( ) c7(CT); #c1(BTN2A2) c2(XP_011512531) c3(butyrophilin subfamily 2 member A2 isoform X4) c4( ) c5(303192) c6( ) c7(CT); #c1(BTN3A1) c2(NP_001138480) c3(butyrophilin subfamily 3 member A1 isoform c precursor) c4(56882) c5(56883, 56884, 56885, 56886, 56887, 56888) c6( ) c7(t, m); #c1(BTN3A1) c2(NP_001138481) c3(butyrophilin subfamily 3 member A1 isoform d precursor) c4(56889, 56890, 56891) c5(56892, 56893, 56894, 56895, 56896, 56897) c6( ) c7(t, m); #c1(BTN3Al) c2(NP_008979) c3(butyrophilin subfamily 3 member A1 isoform a precursor) c4(170008, 170009) c5(170010, 170011, 170012, 170013, 170014, 170015, 170016) c6( ) c7(t, m); #c1(BTN3A1) c2(NP_919423) c3(butyrophilin subfamily 3 member A1 isoform b precursor) c4(258568) c5(258569, 258570, 258571, 258572, 258573, 258574, 258575, 258576, 258577, 258578, 258579, 258580, 258581, 258582, 258583, 258584, 258585, 258586, 258587, 258588, 258589, 258590, 258591, 258592) c6( ) c7(t, m); #c1(BTN3A2) c2(NP_001184175) c3(butyrophilin subfamily 3 member A2 isoform a precursor) c4(75513, 75514, 75515) c5(75516, 75517, 75518, 75519, 75520, 75521, 75522, 75523, 75524, 75525, 75526, 75527, 75528, 75529, 75530, 75531, 75532, 75533, 75534, 75535, 75536, 75537, 75538) c6( ) c7(T, A, aE, af, eZ, r, kp, m, t, q); #c1(BTN3A2) c2(NP_001184/77) c3(butyrophilin subfamily 3 member A2 isoform b) c4(75539) c5(75540, 75541, 75542, 75543, 75544, 75545, 75546) c6( ) c7(T, A, aE, af, eZ, r, kp, m, t, q); #c1(BTN3A2) c2(NP_001184178) c3(butyrophilin subfamily 3 member A2 isoform c) c4( ) c5(75547, 75548, 75549, 75550, 75551) c6( ) c7(T, A, aE, af, eZ, r, kp, m, t, q); #c1(BTN3A2) c2(XP_011512573) c3(butyrophilin subfamily 3 member A2 isoform X5) c4( ) c5(303482, 303483, 303484, 303485, 303486, 303487) c6( ) c7(T, A, aE, af, eZ, r, kp, m, t, q); #c1(BTN3A3) c2(NP_008925) c3(butyrophilin subfamily 3 member A3 isoform a precursor) c4(169569, 169570, 169571, 169572) c5(169573, 169574, 169575, 169576, 169577, 169578, 169579, 169580, 169581, 169582, 169583, 169584, 169585, 169586, 169587, 169588, 169589, 169590, 169591) c6( ) c7(T, aE, awx, r, kp, q); #c1(BTN3A3) c2(NP_932078) c3(butyrophilin subfamily 3 member A3 isoform b) c4(258820) c5(258821, 258822, 258823, 258824, 258825) c6( ) c7(T, aE, awx, r, kp, q); #c1(BTNL2) c2(NP_001291490) c3(butyrophilin-like protein 2) c4(116146, 116147, 116148, 116149, 116150, 116151, 116152, 116153, 116154, 116155, 116156, 116157) c5(116158, 116159, 116160, 116161, 116162, 116163, 116164, 116165, 116166, 116167, 116168, 116169, 116170, 116171, 116172, 116173, 116174, 116175, 116176, 116177, 116178, 116179, 116180, 116181, 116182, 116183, 116184, 116185, 116186, 116187, 116188, 116189, 116190, 116191, 116192, 116193, 116194, 116195, 116196, 116197, 116198, 116199, 116200, 116201, 116202, 116203, 116204, 116205, 116206, 116207, 116208, 116209, 116210, 116211, 116212, 116213, 116214, 116215, 116216, 116217, 116218, 116219, 116220, 116221, 116222, 116223, 116224, 116225, 116226, 116227, 116228, 116229, 116230, 116231, 116232, 116233, 116234, 116235, 116236, 116237, 116238, 116239, 116240, 116241, 116242, 116243, 116244, 116245, 116246, 116247, 116248, 116249, 116250, 116251, 116252, 116253, 116254, 116255, 116256, 116257, 116258, 116259, 116260, 116261, 116262, 116263, 116264, 116265, 116266, 116267, 116268, 116269, 116270, 116271, 116272, 116273, 116274, 116275, 116276, 116277, 116278, 116279, 116280, 116281, 116282, 116283, 116284, 116285, 116286, 116287) c6( ) c7(oe, awz, qj, V, ad, AW, hD, ji, an, ae, bc, ul, A, awy, cr, eZ, fe, iV, aal, pt, ig, uy, lf, y); #c1(BTNL2) c2(XP_011513057) c3 (butyrophilin-like protein 2 isoform X1) c4( ) c5(304650, 304651, 304652, 304653, 304654, 304655, 304656, 304657, 304658, 304659, 304660, 304661, 304662, 304663, 304664) c6( ) c7(oe, awz, qj, V, ad, AW, hD, ji, an, ae, bc, ul, A, awy, cr, eZ, fe, iV, aaD, pt, ig, uy, lf, y); #c1(BTNL2) c2(XP_011513058) c3(butyrophilin-like protein 2 isoform X2) c4(304665, 304666, 304667) c5(304668, 304669, 304670, 304671, 304672, 304673, 304674, 304675, 304676, 304677, 304678, 304679, 304680, 304681, 304682, 304683, 304684, 304685, 304686, 304687, 304688, 304689, 304690, 304691, 304692, 304693, 304694, 304695, 304696, 304697, 304698, 304699, 304700, 304701, 304702, 304703, 304704, 304705, 304706, 304707, 304708, 304709, 304710, 304711, 304712, 304713, 304714, 304715, 304716, 304717, 304718, 304719, 304720, 304721, 304722, 304723, 304724, 304725, 304726, 304727, 304728, 304729, 304730, 304731, 304732, 304733, 304734, 304735, 304736, 304737, 304738) c6( ) c7(oe, awz, qj, V, ad, AW, hD, ji, an, ae, bc, ul, A, awy, cr, eZ, fe, iV, aaQ, pt, ig, uy, lf, y); #c1(BTNL2) c2(XP_011546755) c3(butyrophilin-like protein 2 isoform X1) c4(345672) c5(345673, 345674, 345675, 345676, 345677, 345678, 345679, 345680, 345681, 345682, 345683, 345684) c6( ) c7(oe, awz, qj, V, ad, AW, hD, ji, an, ae, bc, ul, A, awy, cr, eZ, fe, iV, aaO, pt, ig, uy, lf, y); #c1(BTNL2) c2(XP_011546879) c3(butyrophilin-like protein 2 isoform X2) c4(345739) c5(345740, 345741, 345742, 345743, 345744, 345745, 345746, 345747, 345748, 345749, 345750, 345751) c6( ) c7(oe, awz, qj, V, ad, AW, hD, ji, an, ae, bc, ul, A, awy, cr, eZ, fe, iV, aaD, pt, ig, uy, lf, y); #c1(BTRC) c2(NP_001243785) c3(F-box/WD repeat-containing protein IA isoform 3) c4(91719) c5(91720, 91721, 91722, 91723, 91724, 91725) c6( ) c7(T, aC, aA, KO, q, ad, av, aE, aT, jf, d, aW, Y, r, aF, as, cm, bz, bF, iO, nM, bt, B); #c1(BTRC) c2(NP_003930) c3(F-box/WD repeat-containing protein (A isoform 2) c4(142254, 142255) c5(142256, 142257, 142258, 142259, 142260, 142261, 142262, 142263, 142264, 142265, 142266, 142267) c6( ) c7(T, aC, aA, KO, q, ad, av, aE, aT, jf, d, aW, Y, r, aF, as, cm, bz, bF, iO, nM, bt, B); #c1(BTRC) c2(NP_378663) c3(F-box/WD repeat-containing protein (A isoform 1) c4(228926) c5(228927, 228928, 228929, 228930, 228931, 228932, 228933, 228934, 228935, 228936, 228937) c6( ) c7(T, aC, aA, KO, q, ad, av, aE, aT, jf, d, aW, Y, r, aF, as, cm, bz, bF, iO, nM, bt, B); #c1(BTRC) c2(XP_006718117) c3(F-box/WD repeat-containing protein IA isoform X1) c4(291521) c5(291522) c6( ) c7(T, aC, aA, KO, q, ad, av, aE, aT, jf, d, aW, Y, r, aF, as, cm, bz, bF, iO, nM, bt, B); #c1(BUB1) c2(NP_001265545) c3(mitotic checkpoint serine/threonine-protein kinase BUB1 isoform 2) c4(98803) c5(98804, 98805, 98806, 98807, 98808, 98809) c6( ) c7(gh, T, aC, jh, aA, bB, fd, aj, ci, aon, auA, q, ad, x, av, hd, aE, aT, hn, dL, ey, ae, d, je, aW, LZ, r, agF, aF, g, t, iK, bz, dR, ck, iM, eM, nB, bE, m, k, MC, bt, B); #c1(BUB1) c2(NP_001265546) c3(mitotic checkpoint serine/threonine-protein kinase BUB1 isoform 3) c4(98810) c5(98811, 98812, 98813, 98814, 98815, 98816, 98817, 98818, 98819, 98820, 98821, 98822) c6( ) c7(gh, T, aC, jh, aA, bB, fd, aj, ci, aon, auA, q, ad, x, av, hd, aE, aT, hn, dL, ey, ae, d, je, aW, LZ, r, agF, aF, g, t, iK, bz, dR, ck, iM, eM, nB, bE, m, k, MC, bt, B); #c1(BUB1) c2(NP_004327) c3(mitotic checkpoint serine/threonine-protein kinase BUB1 isoform 1) c4(145205, 145206) c5(145207, 145208, 145209, 145210, 145211, 145212) c6( ) c7(gh, T, aC, jh, aA, bB, fd, aj, ci, aon, auA, q, ad, x, av, hd, aE, aT, hn, dl, ey, ae, d, je, aW, LZ, r, agF, aF, g, t, iK, bz, dR, ck, iM, eM, nB, bE, m, k, MC, bt, B); #c1(BUB1B) c2(NP_001202) c3(mitotic checkpoint serine/threonine-protein kinase BUB1 beta) c4(83545, 83546, 83547) c5(83548, 83549, 83550, 83551, 83552, 83553, 83554, 83555, 83556, 83557, 83558, 83559, 83560, 83561, 83562, 83563, 83564, 83565) c6( ) c7(T, fM, aA, it, n, ci, Xr, aon, bv, awA, hd, aE, MR, arV, aT, iv, hP, K, ey, d, je, aiB, Or, qt, Ya, Y, aW, ff, iw, r, aF, aiz, en, cl, g, t, QK, bz, iV, iM, nB, dB, bE, wB, hL, cj, m, bt, xb, B); #c1(BUB3) c2(NP_001007794) c3(mitotic checkpoint protein BUB3 isoform b) c4(22862) c5(22863, 22864, 22865, 22866, 22867, 22868) c6( ) c7(T, eU, d, aW, r, aT, aA, dL, aF, q, eF, B); #c1(BUB3) c2(NP_004716) c3(mitotic checkpoint protein BUB3 isoform a) c4(149036, 149037) c5(149038, 149039, 149040, 149041, 149042, 149043, 149044, 149045, 149046, 149047, 149048, 149049) c6( ) c7(T, el, d, aW, r, aT, aA, dL, aF, q, eF, B); #c1(BUD13) c2(NP_001153208) c3(BUD13 homolog isoform 2) c4(60543) c5(60544, 60545, 60546, 60547, 60548, 60549) c6( ) c7(bL, ae, om, e, ad); #c1(BUD13) c2(NP_116114) c3(BUD13 homolog isoform 1) c4(225360) c5(225361, 225362, 225363, 225364, 225365, 225366) c6( ) c7(bL, ae, om, e, ad); #c1(BUD31) c2(NP_003901) c3(protein BUD31 homolog) c4(142007, 142008) c5(142009, 142010, 142011, 142012, 142013, 142014, 142015, 142016, 142017, 142018, 142019, 142020, 142021, 142022, 142023, 142024) c6( ) c7(T, aW, oe, eH, K, m, bU, eF, t); #c1(BVES) c2(XP_011533700) c3(blood vessel epicardial substance isoform X1) c4(329768) c5(329769, 329770, 329771, 329772, 329773, 329774, 329775, 329776, 329777, 329778, 329779, 329780, 329781, 329782, 329783) c6( ) c7(bE, awB, bB, q, B, adz); #c1(BYSL) c2(NP_004044) c3(bystin) c4(143116) c5(143117, 143118, 143119, 143120, 143121, 143122) c6( ) c7(awC); #c1(BZRAP1) c2(NP_001248764) c3(peripheral-type benzodiazepine receptor-associated protein 1 isoform c) c4( ) c5(94016) c6( ) c7(bP, Mb, eA, K, bN); #c1(BZRAP1) c2(NP_004749) c3(peripheral-type benzodiazepine receptor-associated protein 1 isoform a) c4(149247) c5(149248, 149249, 149250, 149251, 149252, 149253, 149254) c6( ) c7(bP, Mb, eA, K, bN); #c1(BZRAP1) c2(NP_077729) c3(peripheral-type benzodiazepine receptor-associated protein 1 isoform b) c4(215314) c5(215315, 215316, 215317, 215318, 215319, 215320, 215321) c6( ) c7(bP, Mb, eA, K, bN); #c1(BZRAP1) c2(XP_006722236) c3(peripheral-type benzodiazepine receptor-associated protein 1 isoform X1) c4(294661) c5(294662, 294663, 294664, 294665, 294666, 294667, 294668) c6( ) c7(bP, Mb, eA, K, bN); #c1(BZRAP1) c2(XP_011523774) c3(peripheral-type benzodiazepine receptor-associated protein 1 isoform X8) c4( ) c5(318268, 318269) c6( ) c7(bP, Mb, eA, K, bN); #c1(BZW1) c2(NP_001193996) c3(basic leucine zipper and W2 domain-containing protein 1 isoform 1) c4(83463) c5(83464, 83465, 83466, 83467, 83468, 83469, 83470, 83471, 83472, 83473) c6( ) c7(Y); #c1(BZW1) c2(NP_001193997) c3(basic leucine zipper and W2 domain-containing protein 1 isoform 2) c4(83474, 83475) c5(83476, 83477, 83478, 83479, 83480, 83481, 83482, 83483) c6( ) c7(Y); #c1(BZW1) c2(NP_001193998) c3(basic leucine zipper and W2 domain-containing protein 1 isoform 3) c4(83484, 83485, 83486, 83487) c5(83488, 83489, 83490, 83491, 83492, 83493, 83494, 83495, 83496) c6( ) c7(Y); #c1(BZW1) c2(NP_055485) c3(basic leucine zipper and W2 domain-containing protein 1 isoform 4) c4(181412) c5(181413, 181414, 181415, 181416, 181417, 181418, 181419, 181420, 181421, 181422, 181423, 181424, 181425, 181426, 181427, 181428, 181429, 181430, 181431, 181432, 181433) c6( ) c7(Y); #c1(C10orf10) c2(NP_008952) c3(protein DEPP) c4(169727, 169728, 169729) c5(169730, 169731, 169732, 169733, 169734, 169735, 169736, 169737, 169738, 169739, 169740, 169741, 169742, 169743, 169744, 169745, 169746, 169747) c6( ) c7(dP, d, aW, bo, aT, awD, v, aS, K, du); #c1(C10orf107) c2(XP_005269657) c3(uncharacterized protein C10orf107 isoform X1) c4(282687, 282688) c5(282689, 282690, 282691, 282692, 282693) c6( ) c7(oe, cz); #c1(C10orf107) c2(XP_0067/7753) c3(uncharacterized protein C10orf107 isoform X2) c4( ) c5(291314) c6( ) c7(oe, cz); #c1(C10orf107) c2(XP_0067/7755) c3(uncharacterized protein C10orf107 isoform X4) c4( ) c5(291315, 291316, 291317, 291318, 291319, 291320) c6( ) c7(oe, cz); #c1(C10orf11) c2(NP_114413) c3(leucine-rich repeat-containing protein C10orf11) c4(222661) c5(222662, 222663, 222664, 222665, 222666, 222667) c6( ) c7(aW, awE, aT, n, j, lB); #c1(C10orf113) c2(NP_001010896) c3(putative uncharacterized protein C10orf113 isoform 1) c4(24014, 24015) c5(24016, 24017, 24018, 24019, 24020, 24021, 24022, 24023, 24024, 24025, 24026, 24027, 24028) c6( ) c7(aw, j); #c1(C10orf113) c2(NP_001170954) c3(putative uncharacterized protein C10orf113 isoform 2) c4(69614) c5(69615, 69616, 69617, 69618, 69619) c6( ) c7(aw, j); #c1(C10orf2) c2(NP_001157284) c3(twinkle protein, mitochondrial isoform B) c4(62142) c5(62143, 62144, 62145, 62146, 62147, 62148) c6( ) c7(awH, awG, awf, qx, amp, ha, rf, fr, ci, fu, V, aoz, cc, no, KB, oj, yZ, bq, bV, awL, an, af, Ng, KG, A, gl, EH, ir, RI, awJ, t, kC, cm, bw, dR, akQ, ky, kB, Kv, awN, aaG, U, awM, awk, bk, m, aan, awl, Al); #c1(C10orf2) c2(NP_068602) c3(twinkle protein, mitochondrial isoform A) c4(209775) c5(209776, 209777, 209778, 209779, 209780, 209781) c6( ) c7(awH, awG, awf, qx, amp, ha, rf, fr, ci, fu, V, aoz, cc, no, KB, oj, yZ, bq, bV, awL, an, af, Ng, KG, A, gl, EH, ir, RI, awJ, t, kC, cm, bw, dR, akl, ky, KB, Kv, awN, aaG, U, awM, awK, bk, m, aan, awl, A1); #c1(C10orf32) c2(NP_001129672) c3(UPF0693 protein C10orf32) c4(52183) c5(52184, 52185, 52186, 52187, 52188, 52189, 52190, 52191, 52192, 52193) c6( ) c7(an); #c1(C10orf35) c2(NP_660349) c3(uncharacterized protein C10orf35) c4( ) c5(241042, 241043, 241044, 241045, 241046) c6( ) c7(af); #c1(C10orf35) c2(XP_005269663) c3(uncharacterized protein C10orf35 isoform X2) c4(282694, 282695) c5(282696, 282697, 282698, 282699, 282700, 282701, 282702) c6( ) c7(af); #c1(C10orf54) c2(NP_071436) c3(platelet receptor Gi24 precursor) c4(211659, 211660) c5(211661, 211662, 211663, 211664, 211665, 211666, 211667, 211668, 211669, 211670, 211671, 211672) c6( ) c7(bM); #c1(C10orf67) c2(NP_714925) c3 (uncharacterized protein C10orf67) c4( ) c5(246632, 246633, 246634, 246635, 246636, 246637) c6( ) c7(dB, iV, aC, uQ); #c1(C10orf67) c2(XP_011517743) c3(uncharacterized protein C10orf67 isoform X2) c4(311551, 311552) c5( ) c6( ) c7(dB, iV, aC, uQ); #c1(C10orf67) c2(XP_011517744) c3(uncharacterized protein C10orf67 isoform X3) c4( ) c5(311553, 311554, 311555, 311556, 311557, 311558) c6( ) c7(dB, iV, aC, uQ); #c1(C10orf67) c2(XP_011517745) c3(uncharacterized protein C10orf67 isoform X4) c4(311559) c5(311560, 311561, 311562, 311563, 311564, 311565, 311566, 311567, 311568, 311569) c6( ) c7(dB, iV, aC, uQ); #c1(C10orf82) c2(NP_653262) c3(uncharacterized protein C10orf82) c4( ) c5(239386, 239387, 239388, 239389, 239390) c6( ) c7(IB); #c1(C10orf82) c2(XP_011537642) c3 (uncharacterized protein C10orf82 isoform X1) c4(335200) c5( ) c6( ) c7(IB); #c1(C10orf82) c2(XP_011537648) c3 (uncharacterized protein C10orf82 isoform X4) c4(335201) c5(335202, 335203, 335204, 335205, 335206, 335207, 335208) c6( ) c7(IB); #c1(C10orf88) c2(NP_079218) c3(uncharacterized protein C10orf88) c4(217481) c5(217482, 217483, 217484, 217485, 217486, 217487) c6( ) c7(aW, eA, aT); #c1(C10orf90) c2(NP_001004298) c3(centrosomal protein C10orf90) c4(20491, 20492, 20493) c5(20494, 20495, 20496, 20497, 20498, 20499, 20500, 20501, 20502, 20503, 20504, 20505, 20506, 20507, 20508, 20509, 20510, 20511, 20512, 20513, 20514, 20515, 20516, 20517) c6( ) c7(T, fq, j, ac); #c1(C10orf90) c2(XP_005269548) c3(centrosomal protein C10orf90 isoform X2) c4( ) c5(282633) c6( ) c7(T, fq, j, ac); #c1(C10orf90) c2(XP_011537518) c3(centrosomal protein C10orf90 isoform X1) c4(335120, 335121, 335122) c5(335123, 335124, 335125, 335126, 335127, 335128, 335129) c6( ) c7(T, fq, j, ac); #c1(C10orf90) c2(XP_011537531) c3(centrosomal protein C10orf90 isoform X5) c4( ) c5(335130, 335131, 335132, 335133, 335134, 335135, 335136, 335137) c6( ) c7(T, fq, j, ac); #c1(C10orf90) c2(XP_011537539) c3(centrosomal protein C10orf90 isoform X8) c4(335138) c5(335139, 335140, 335141, 335142, 335143, 335144) c6( ) c7(T, fq, j, ac); #c1(C11orf21) c2(NP_001136418) c3(uncharacterized protein C11orf21) c4(54939, 54940) c5(54941, 54942, 54943, 54944, 54945, 54946, 54947, 54948, 54949, 54950, 54951, 54952) c6( ) c7(cj); #c1(C11orf30) c2(NP_064578) c3(protein EMSY isoform 4) c4(203066, 203067) c5(203068, 203069, 203070, 203071, 203072, 203073, 203074, 203075, 203076, 203077, 203078, 203079, 203080, 203081, 203082, 203083) c6( ) c7(bM, T, kl, qw, iV, nB, aW, aT, fe, ci, m, cl, eE, t, ae, ad); #c1(C11orf30) c2(XP_006718700) c3(protein EMSY isoform X9) c4(291986) c5( ) c6( ) c7(bM, T, kl, qw, iV, nB, aW, aT, fe, ci, m, cl, eE, t, ae, ad); #c1(C11orf30) c2(XP_006718702) c3(protein EMSY isoform X12) c4( ) c5(291987, 291988, 291989, 291990, 291991, 291992) c6( ) c7(bM, T, kl, qw, iV, nB, aW, aT, fe, ci, m, cl, eE, t, ae, ad); #c1(C11orf31) c2(NP_734467) c3(selenoprotein H) c4(247299, 247300) c5(247301, 247302, 247303, 247304, 247305, 247306, 247307, 247308, 247309, 247310, 247311, 247312, 247313, 247314, 247315, 247316) c6( ) c7(anN); #c1(C11orf53) c2(NP_940900) c3(uncharacterized protein C11orf53) c4( ) c5(260071, 260072, 260073, 260074, 260075) c6( ) c7(q, r); #c1(C11orf53) c2(XP_011541105) c3(uncharacterized protein C11orf53 isoform X2) c4(339619) c5(339620) c6( ) c7(q, r); #c1(C11orf65) c2(NP_689800) c3(uncharacterized protein C11orf65) c4(243984) c5(243985, 243986, 243987, 243988, 243989, 243990, 243991, 243992, 243993, 243994) c6( ) c7(dl); #c1(C11orf68) c2(NP_001129107) c3(UPF0696 protein C11orf68 isoform 1) c4(50771) c5(50772, 50773, 50774, 50775, 50776, 50777) c6( ) c7(Y); #c1(C11orf68) c2(NP_113638) c3(UPF0696 protein C11orf68 isoform 2) c4(221843) c5(221844, 221845, 221846, 221847, 221848, 221849) c6( ) c7(Y); #c1(C11orf73) c2(NP_057485) c3(protein Hikeshi) c4(191150) c5(191151, 191152, 191153, 191154, 191155, 191156) c6( ) c7(Y, aA); #c1(C11orf74) c2(NP_001263656) c3(uncharacterized protein C11orf74 isoform b) c4(97384, 97385) c5(97386, 97387, 97388, 97389, 97390, 97391, 97392, 97393, 97394, 97395, 97396, 97397, 97398, 97399, 97400, 97401) c6( ) c7(Kc, fq, pN); #c1(C11orf74) c2(XP_011518187) c3(uncharacterized protein C11orf74 isoform X1) c4(312202, 312203, 312204, 312205, 312206) c5(312207, 312208, 312209, 312210, 312211, 312212, 312213, 312214, 312215, 312216, 312217, 312218, 312219, 312220, 312221, 312222, 312223, 312224, 312225, 312226, 312227, 312228, 312229, 312230, 312231, 312232, 312233, 312234, 312235, 312236) c6( ) c7(Kc, fq, pN); #c1(C11orf80) c2(NP_001289013) c3(uncharacterized protein C11orf80 isoform b) c4(114116) c5(114117, 114118, 114119, 114120, 114121, 114122) c6( ) c7(bK); #c1(C11orf80) c2(NP_078926) c3(uncharacterized protein C11orf80 isoform a) c4(216155, 216156) c5(216157, 216158, 216159, 216160, 216161, 216162, 216163, 216164, 216165, 216166, 216167, 216168) c6( ) c7(bk); #c1(C11orf87) c2(XP_011541119) c3 (uncharacterized protein C11orf87 isoform X2) c4(339636) c5(339637) c6( ) c7(oe, ac); #c1(C11orf87) c2(XP_011541120) c3(uncharacterized protein C11orf87 isoform X1) c4( ) c5(339638, 339639, 339640, 339641, 339642) c6( ) c7(oe, ac); #c1(C11orf95) c2(NP_001138408) c3(uncharacterized protein C11orf95) c4( ) c5(56576) c6( ) c7(awl, awP, hj); #c1(C12orf10) c2(NP_067653) c3(UPF0160 protein MYG1, mitochondrial precursor) c4(209240, 209241) c5(209242, 209243, 209244, 209245, 209246, 209247, 209248, 209249, 209250, 209251, 209252, 209253) c6( ) c7(yl, aW, aT, awQ, qj, pC); #c1(C12orf42) c2(NP_940923) c3 (uncharacterized protein C12orf42 isoform 1) c4( ) c5(260117, 260118, 260119, 260120, 260121, 260122, 260123, 260124, 260125, 260126, 260127, 260128, 260129) c6( ) c7(iy); #c1(C12orf42) c2(XP_011536593) c3(uncharacterized protein C12orf42 isoform X2) c4(334092, 334093) c5(334094, 334095, 334096) c6( ) c7(iy); #c1(C12orf42) c2(XP_011536616) c3(uncharacterized protein C12orf42 isoform X15) c4(334097) c5( ) c6( ) c7(iy); #c1(C12orf42) c2(XP_011536619) c3(uncharacterized protein C12orf42 isoform X17) c4( ) c5(334098, 334099, 334100, 334101, 334102, 334103) c6( ) c7(iy); #c1(C12orf43) c2(NP_001273127) c3(uncharacterized protein C12orf43 isoform g) c4(104329) c5(104330, 104331, 104332, 104333, 104334, 104335) c6( ) c7(j); #c1(C12orf43) c2(NP_075046) c3 (uncharacterized protein C12orf43 isoform c) c4(213557, 213558) c5(213559, 213560, 213561, 213562, 213563, 213564, 213565, 213566, 213567, 213568, 213569, 213570) c6( ) c7(j); #c1(C12orf5) c2(NP_065108) c3(fructose-2,6-bisphosphatase TIGAR) c4(203959) c5(203960, 203961, 203962, 203963, 203964, 203965) c6( ) c7(T, d, bP, X, ja, cj, aq, ey, K, eA, iV, KV, B); #c1(C12orf50) c2(NP_689802) c3(uncharacterized protein C12orf50) c4(244002, 244003) c5(244004, 244005, 244006, 244007, 244008) c6( ) c7(j); #c1(C12orf50) c2(XP_011536284) c3(uncharacterized protein C12orf50 isoform X1) c4( ) c5(333620) c6( ) c7(j); #c1(C12orf50) c2(XP_011536287) c3(uncharacterized protein C12orf50 isoform X4) c4( ) c5(333621, 333622, 333623, 333624, 333625, 333626) c6( ) c7(j); #c1(C12orf57) c2(NP_001288763) c3(protein CIO isoform 1) c4(114012) c5(114013, 114014, 114015, 114016, 114017, 114018) c6( ) c7(awR, af, afy, nA, awS); #c1(C12orf65) c2(NP_001181924) c3(probable peptide chain release factor C12orf65, mitochondrial) c4(74393, 74394, 74395, 74396) c5(74397, 74398, 74399, 74400, 74401, 74402, 74403, 74404, 74405, 74406, 74407, 74408, 74409, 74410, 74411, 74412, 74413, 74414, 74415, 74416, 74417, 74418, 74419, 74420, 74421, 74422, 74423, 74424, 74425, 74426, 74427, 74428) c6( ) c7(awT, awk, px, fu, awl); #c1(C12orf66) c2(NP_001287869) c3(UPF0536 protein C12orf66 isoform 1) c4(113102) c5( ) c6( ) c7(kl); #c1(C12orf66) c2(NP_689653) c3(UPF0536 protein C12orf66 isoform 2) c4( ) c5(243480, 243481, 243482, 243483, 243484, 243485) c6( ) c7(kl); #c1(C12orf75) c2(NP_001138671) c3(overexpressed in colon carcinoma 1 protein) c4(57336) c5(57337, 57338, 57339, 57340, 57341, 57342) c6( ) c7(t, awV, e, m, pN); #c1(C12orf77) c2(NP_001094809) c3(putative uncharacterized protein C12orf77) c4(40758) c5(40759, 40760, 40761, 40762, 40763, 40764) c6( ) c7(aw); #c1(C14orf1) c2(NP_009107) c3(probable ergosterol biosynthetic protein 28) c4(170700) c5(170701, 170702, 170703, 170704, 170705, 170706) c6( ) c7(aE); #c1(C14orf166) c2(NP_057123) c3(UPF0568 protein C14orf166) c4(189002) c5(189003, 189004, 189005, 189006, 189007, 189008, 189009) c6( ) c7(dB, aC); #c1(C14orf177) c2(NP_872366) c3(putative uncharacterized protein C14orf177) c4(256244, 256245) c5(256246, 256247, 256248, 256249, 256250, 256251, 256252, 256253, 256254, 256255, 256256, 256257, 256258) c6( ) c7(cd); #c1(C15orf32) c2(NP_694585) c3(uncharacterized protein C15orf32 isoform a) c4(245330) c5(245331, 245332, 245333, 245334, 245335, 245336) c6( ) c7(bK); #c1(C15orf41) c2(XP_011520412) c3(uncharacterized protein C15orf41 isoform X1) c4(315298, 315299) c5(315300, 315301, 315302, 315303, 315304, 315305, 315306, 315307) c6( ) c7(awW); #c1(C15orf41) c2(XP_011520414) c3(uncharacterized protein C15orf41 isoform X2) c4( ) c5(315308, 315309, 315310, 315311, 315312, 315313, 315314, 315315) c6( ) c7(awW); #c1(C15orf48) c2(NP_922946) c3(normal mucosa of esophagus-specific gene 1 protein) c4(258713, 258714) c5(258715, 258716, 258717, 258718, 258719, 258720, 258721, 258722, 258723, 258724, 258725) c6( ) c7(T, it, ff, en); #c1(C15orf53) c2(NP_997327) c3(uncharacterized protein C15orf53) c4(265411) c5(265412, 265413, 265414, 265415, 265416, 265417) c6( ) c7(cd, oe, bK, ac, ad); #c1(C15orf59) c2(XP_005254426) c3(UPF0583 protein C15orf59 isoform X1) c4(273197) c5(273198, 273199, 273200, 273201, 273202, 273203, 273204, 273205, 273206, 273207) c6( ) c7(fq, ad); #c1(C16orf72) c2(NP_054836) c3(UPF0472 protein C16orf72) c4(178110) c5(178111, 178112, 178113, 178114, 178115, 178116) c6( ) c7(Ea); #c1(C16orf74) c2(NP_996850) c3(uncharacterized protein C16orf74) c4(264782) c5(264783, 264784, 264785, 264786, 264787, 264788) c6( ) c7(eL, dL); #c1(C16orf78) c2(NP_653203) c3(uncharacterized protein C16orf78) c4(239050) c5(239051, 239052, 239053, 239054, 239055, 239056) c6( ) c7(EQ); #c1(C16orf95) c2(NP_001182053) c3(uncharacterized protein C16orf95 isoform 1) c4(74514) c5(74515, 74516, 74517, 74518, 74519, 74520) c6( ) c7(awX); #c1(C16orf95) c2(NP_001182054) c3(uncharacterized protein C16orf95 isoform 2) c4(74521) c5(74522, 74523, 74524, 74525, 74526, 74527) c6( ) c7(awX); #c1(C17orf51) c2(NP_001106905) c3(uncharacterized protein C17orf51) c4(42678) c5(42679, 42680, 42681, 42682, 42683, 42684) c6( ) c7(cr, an); #c1(C17orf53) c2(NP_076937) c3(uncharacterized protein C17orf53 isoform 1) c4(214306) c5(214307, 214308, 214309, 214310, 214311, 214312) c6( ) c7(D); #c1(C17orf53) c2(XP_011523493) c3(uncharacterized protein C17orf53 isoform X5) c4(318019) c5( ) c6( ) c7(D); #c1(C17orf53) c2(XP_011523498) c3(uncharacterized protein C17orf53 isoform X10) c4(318020) c5(318021, 318022, 318023, 318024, 318025, 318026,

318027, 318028, 318029, 318030, 318031, 318032) c6( ) c7(D); #c1(C17orf64) c2(NP_859058) c3(uncharacterized protein C17orf64) c4(255434) c5(255435, 255436, 255437, 255438, 255439, 255440) c6( ) c7(W); #c1(C17orf96) c2(NP_001124149) c3 (uncharacterized protein C17orf96) c4(48291) c5(48292, 48293, 48294, 48295, 48296, 48297, 48298) c6( ) c7(bX); #c1(C18orf54) c2(NP_001275909) c3(lung adenoma susceptibility protein 2 isoform a) c4(107369) c5(107370, 107371, 107372, 107373, 107374, 107375) c6( ) c7(av, auM, ac, g); #c1(C18orf54) c2(NP_775800) c3(lung adenoma susceptibility protein 2 isoform b precursor) c4(250341) c5(250342, 250343, 250344, 250345, 250346, 250347) c6( ) c7(av, auM, ac, g); #c1(C18orf8) c2(NP_001263271) c3(uncharacterized protein C18orf8 isoform 2) c4( ) c5(97124, 97125) c6( ) c7(bL, T, dD, aiR, ea, e, aw, aA, bB, n, v, eq, q, ad, eA, aE, sO, aT, dL, K, KV, ae, eL, cp, d, aW, Y, aF, pl, ar, pu, ac, bE, y, LS, awY); #c1(C18orf8) c2(NP_037458) c3(uncharacterized protein C18orf8 isoform 1) c4(175612) c5(175613, 175614, 175615, 175616, 175617, 175618, 175619, 175620, 175621, 175622, 175623, 175624, 175625) c6( ) c7(bL, T, dD, aiR, ea, e, aw, aA, bB, n, v, eq, q, ad, eA, aE, sO, aT, dL, K, KV, ae, eL, cp, d, aW, Y, aF, pl, ar, pu, ac, bE, y, LS, awY); #c1(C19 orf12) c2(NP_001026896) c3(protein C19orf12 isoform 1) c4(29924, 29925) c5(29926, 29927, 29928, 29929, 29930, 29931, 29932) c6( ) c7(axa, Pw, aV, axb, dL, awZ, t); #c1(C19orf(2) c2(NP_001242975) c3(protein C19orf12 isoform 3) c4(89459) c5(89460, 89461, 89462, 89463, 89464, 89465) c6( ) c7(axa, Pw, aV, axb, dL, awZ, t); #c1(C19orf12) c2(NP_113636) c3 (protein C19orf12 isoform 2) c4( ) c5(221838, 221839, 221840, 221841, 221842) c6( ) c7(axa, Pw, aV, axb, dL, awZ, t); #c1(C19orf18) c2(NP_689687) c3(uncharacterized protein C19orf18 precursor) c4(243574) c5(243575, 243576, 243577, 243578, 243579, 243580) c6( ) c7(IB); #c1(C19orf24) c2(NP_060384) c3(uncharacterized membrane protein C19orf24 precursor) c4(195736) c5(195737, 195738, 195739, 195740, 195741, 195742, 195743, 195744, 195745) c6(195746, 195747) c7(aw); #c1(C19orf26) c2(NP_689982) c3(protein Dos) c4(244645, 244646) c5(244647, 244648, 244649, 244650, 244651, 244652, 244653, 244654, 244655, 244656, 244657, 244658) c6( ) c7(T, aog, axc); #c1(C19orf33) c2(NP_277055) c3(immortalization up-regulated protein) c4(228881) c5(228882, 228883, 228884, 228885, 228886, 228887) c6( ) c7(n, ji, hL, eb); #c1(C19orf40) c2(NP_689479) c3(Fanconi anemia-associated protein of 24 kDa isoform 1) c4(242830) c5(242831, 242832, 242833, 242834, 242835, 242836, 242837, 242838, 242839, 242840) c6( ) c7(oZ, os, K); #c1(C19orf45) c2(XP_011526294) c3(uncharacterized protein C19orf45 isoform X1) c4(320566, 320567) c5(320568, 320569, 320570, 320571, 320572, 320573) c6( ) c7(ik); #c1(C19orf45) c2(XP_011526296) c3 (uncharacterized protein C19orf45 isoform X3) c4(320574) c5( ) c6( ) c7(iK); #c1(C19orf45) c2(XP_011526297) c3(uncharacterized protein C19orf45 isoform X4) c4( ) c5(320575, 320576, 320577, 320578, 320579, 320580, 320581, 320582, 320583, 320584, 320585, 320586) c6( ) c7(iK); #c1(C19orf48) c2(NP_001277083) c3(uncharacterized protein C19orf48) c4(109012) c5(109013, 109014, 109015, 109016, 109017, 109018, 109019, 109020, 109021, 109022) c6( ) c7(es, T, bE, aW, Y, iB, cj, aT, bB, fr, bt, B, er); #c1(C19orf57) c2(NP_077299) c3(uncharacterized protein C19orf57) c4(215101, 215102) c5(215103, 215104, 215105, 215106, 215107, 215108, 215109, 215110, 215111, 215112, 215113, 215114) c6( ) c7(ac); #c1(C19orf57) c2(XP_011526589) c3 (uncharacterized protein C19orf57 isoform X2) c4( ) c5(320895) c6( ) c7(ac); #c1(C19orf68) c2(NP_955373) c3(uncharacterized protein C19orf68) c4(261590) c5(261591, 261592, 261593, 261594, 261595, 261596) c6( ) c7(kl); #c1(CID) c2(NP_775269) c3(nuclear nucleic acid-binding protein CID) c4(249724, 249725) c5(249726, 249727, 249728, 249729, 249730, 249731, 249732, 249733, 249734, 249735, 249736, 249737, 249738, 249739, 249740, 249741, 249742, 249743, 249744, 249745) c6( ) c7(bL, T, dD, vl, ea, axd, aw, cz, v, uu, ci, j, eq, ip, av, aT, af, bA, bf, cW, aW, Y, g, acD, bz, Be, aS, iu, L); #c1 (C1GALT1) c2(XP_005249869) c3 (glycoprotein-N-acetylgalactosamine 3-beta-galactosyltransferase 1 isoform X2) c4(269937, 269938) c5(269939, 269940, 269941, 269942) c6( ) c7(T, HZ, bE, dM, bB, bK, dy, uE, ey, hH, V, B); #c1(C1GALT1) c2(XP_011513758) c3(glycoprotein-N-acetylgalactosamine 3-beta-galactosyltransferase 1 isoform X1) c4(306106, 306107) c5(306108, 306109, 306110, 306111, 306112, 306113, 306114, 306115, 306116, 306117, 306118, 306119, 306120, 306121, 306122, 306123, 306124, 306125, 306126, 306127, 306128, 306129, 306130, 306131) c6( ) c7(T, HZ, bE, dM, bB, bK, dy, uE, ey, hH, V, B); #c1(C1GALT1C1) c2(NP_689905) c3(C1GALT1-specific chaperone 1) c4(244332) c5(244333, 244334, 244335, 244336, 244337, 244338, 244339, 244340, 244341, 244342) c6( ) c7(HZ, ey, Jn, X, Jm); #c1(C1orf106) c2(NP_001136041) c3(uncharacterized protein C1orf106 isoform 2) c4(54140, 54141) c5(54142, 54143, 54144, 54145, 54146, 54147, 54148, 54149, 54150, 54151, 54152) c6( ) c7(fe, iV); #c1(C1orf106) c2(NP_060735) c3(uncharacterized protein C1orf106 isoform 1) c4(197538) c5(197539, 197540, 197541, 197542, 197543, 197544, 197545, 197546) c6( ) c7(fe, iV); #c1(C1orf106) c2(XP_011508058) c3(uncharacterized protein C1orf106 isoform X2) c4(298525) c5(298526, 298527, 298528, 298529, 298530, 298531, 298532) c6( ) c7(fe, iV); #c1(C1orf109) c2(NP_001289959) c3(uncharacterized protein C1orf109 isoform 2) c4(114870) c5( ) c6( ) c7(aW, T, np, aT); #c1(C1orf109) c2(NP_001289960) c3(uncharacterized protein C1orf109 isoform 1) c4( ) c5(114871) c6( ) c7(aW, T, np, aT); #c1(C1orf110) c2(NP_848645) c3(coiled-coil domain-containing protein C1orf110) c4( ) c5(254069, 254070, 254071, 254072, 254073) c6( ) c7(bL, fe, cd, cz); #c1(C1orf110) c2(XP_005245182) c3(coiled-coil domain-containing protein C1orf110 isoform X2) c4(266339) c5(266340) c6( ) c7(bL, fe, cd, cz); #c1(C1orf110) c2(XP_006711352) c3(coiled-coil domain- containing protein C1orf111 isoform X3) c4( ) c5(285896, 285897, 285898, 285899, 285900) c6( ) c7(bL, fe, cd, cz); #c1(C1orf110) c2(XP_011507779) c3(coiled-coil domain-containing protein C1orf110 isoform X1) c4(297843) c5(297844) c6( ) c7(bL, fe, cd, cz); #c1(C1orf112) c2(XP_005245374) c3(uncharacterized protein C1orf112 isoform X2) c4(266504) c5(266505, 266506, 266507, 266508, 266509, 266510, 266511, 266512) c6( ) c7(T, bA, cz); #c1(C1orf112) c2(XP_011508033) c3(uncharacterized protein C1orf112 isoform X1) c4(298495) c5(298496, 298497, 298498, 298499, 298500, 298501, 298502, 298503, 298504) c6( ) c7(T, bA, cz); #c1(C1orf112) c2(XP_011508036) c3(uncharacterized protein C1orf112 isoform X3) c4(298505) c5(298506, 298507, 298508, 298509, 298510) c6( ) c7(T, bA, cz); #c1(C1orf115) c2(NP_078985) c3(uncharacterized protein C1orf115) c4(216431) c5(216432, 216433, 216434, 216435, 216436, 216437) c6( ) c7(c); #c1(C1orf127) c2(XP_011539059) c3(uncharacterized protein C1orf(27 isoform X5) c4( ) c5(337137, 337138) c6( ) c7(dx); #c1(C1orf141) c2(XP_011539768) c3 (uncharacterized protein C1orf141 isoform X1) c4(337977, 337978) c5(337979, 337980, 337981, 337982, 337983, 337984, 337985, 337986, 337987, 337988) c6( ) c7(AW, HA); #c1(C1orf141) c2(XP_011539769) c3(uncharacterized protein C1orf141 isoform X2) c4(337989) c5( ) c6( ) c7(AW, HA); #c1(C1orf141) c2(XP_011539772) c3(uncharacterized protein C1orf141 isoform X5) c4( ) c5(337990) c6( ) c7(AW, HA); #c1(C1orf141) c2(XP_011539777) c3(uncharacterized protein C1orf141 isoform X10) c4( ) c5(337991, 337992, 337993, 337994, 337995, 337996, 337997, 337998, 337999, 338000, 338001) c6( ) c7(AW, HA); #c1(C1orf167) c2(XP_011539574) c3(uncharacterized protein C1orf167 isoform X3) c4(337801) c5(337802, 337803, 337804, 337805, 337806, 337807) c6( ) c7(bL); #c1(C1orf167) c2(XP_011539579) c3(uncharacterized protein C1orf167 isoform X6) c4(337808) c5(337809) c6( ) c7(bL); #c1(C1orf167) c2(XP_011539581) c3(uncharacterized protein C1orf167 isoform X8) c4( ) c5(337810, 337811, 337812, 337813, 337814, 337815) c6( ) c7(bL); #c1(C1orf204) c2(NP_001127705) c3(uncharacterized protein LOC284677) c4(49258) c5(49259, 49260, 49261, 49262, 49263, 49264) c6( ) c7(bL); #c1(C1orf226) c2(NP_001078844) c3(uncharacterized protein C1orf226 isoform 2) c4( ) c5(37919, 37920, 37921, 37922, 37923, 37924, 37925, 37926, 37927, 37928) c6( ) c7(cd); #c1(C1orf226) c2(NP_001128712) c3(uncharacterized protein C1orf226 isoform 1) c4(50561, 50562, 50563) c5(50564, 50565, 50566, 50567, 50568, 50569, 50570, 50571) c6( ) c7(cd); #c1(C1orf228) c2(XP_011539646) c3(uncharacterized protein C1orf228 isoform X1) c4(337913) c5(337914, 337915, 337916, 337917, 337918, 337919, 337920, 337921, 337922, 337923) c6( ) c7(bM, A, Jr, z, Y, eE, afS, dV, r, cU, Hn, h, cr, Bo, eZ, fe, afT, y, V, q); #c1(C1orf27) c2(NP_001157717) c3(protein odr-4 homolog isoform 2) c4(62456) c5(62457, 62458, 62459, 62460, 62461, 62462) c6( ) c7(x); #c1(C1orf27) c2(NP_001157718) c3(protein odr-4 homolog isoform 3) c4(62463) c5(62464, 62465, 62466, 62467, 62468, 62469) c6( ) c7(x); #c1(C1orf27) c2(XP_011507962) c3(protein odr-4 homolog isoform X1) c4(298385, 298386) c5(298387, 298388, 298389, 298390, 298391, 298392) c6( ) c7(x); #c1(C1orf27) c2(XP_011507964) c3(protein odr-4 homolog isoform X3) c4( ) c5(298393, 298394, 298395, 298396, 298397, 298398) c6( ) c7(x); #c1(C1orf61) c2(XP_005244889) c3(protein CROC-4 isoform X6) c4(266143, 266144) c5(266145, 266146, 266147, 266148, 266149, 266150, 266151, 266152, 266153, 266154, 266155) c6( ) c7(al, T, B, hl); #c1(C1orf61) c2(XP_011507370) c3(protein CROC-4 isoform X3) c4( ) c5(297213) c6( ) c7(al, T, B, hl); #c1(C1orf61) c2(XP_011507374) c3(protein CROC-4 isoform X1) c4(297214, 297215, 297216) c5(297217, 297218, 297219, 297220, 297221, 297222, 297223, 297224, 297225, 297226, 297227, 297228, 297229, 297230, 297231, 297232, 297233, 297234) c6( ) c7(al, T, B, hl); #c1(C1orf86) c2(XP_011539224) c3(Fanconi anemia-associated protein of 20 kDa isoform X1) c4(337333) c5( ) c6( ) c7(oZ, os); #c1(C1QB) c2(XP_011540361) c3(complement C1q subcomponent subunit B isoform X1) c4(338824, 338825, 338826, 338827) c5(338828, 338829, 338830, 338831, 338832, 338833, 338834, 338835, 338836, 338837, 338838, 338839, 338840, 338841, 338842, 338843, 338844, 338845, 338846, 338847, 338848, 338849, 338850, 338851) c6( ) c7(axe, oV, Y, iW, wf, eZ, j, fL, ey, mC); #c1 (C1QBP) c2(NP_001203) c3(complement component 1 Q subcomponent-binding protein, mitochondrial precursor) c4(83566) c5(83567, 83568, 83569, 83570, 83571, 83572) c6( ) c7(T, dA, fR, aW, if, qp, jx, cn, aT, h, jh, fD, qN, cm, bz, B, ip); #c1(C1QL1) c2(NP_006679) c3(C1q-related factor precursor) c4(167096) c5(167097, 167098, 167099, 167100, 167101, 167102) c6( ) c7(T, eR, bO, aj, bS, ct, aY, J, fU, ft, iX, aT, h, K, nJ, af, s, aW, Z, cm, hq, ac, cA, vL, hR); #c1(C1QL3) c2(NP_001010908) c3(complement C1q-like protein 3 precursor) c4(24064, 24065) c5(24066, 24067, 24068, 24069, 24070, 24071, 24072, 24073, 24074, 24075, 24076, 24077, 24078, 24079, 24080, 24081) c6(24082, 24083, 24084, 24085) c7(v); #c1(C1QTNF1) c2(NP_112230) c3(complement C1q tumor necrosis factor-related protein 1 isoform 1 precursor) c4( ) c5(220981, 220982, 220983, 220984, 220985, 220986, 220987, 220988, 220989, 220990, 220991, 220992, 220993) c6(220994, 220995, 220996, 220997) c7(bL, W, pX, fc, hl, cz, Ja, dG, v, cP, ei, Hu); #c1 (C1QTNF1) c2(NP_940996) c3(complement C1q tumor necrosis factor-related protein 1 isoform 2) c4( ) c5(260286, 260287, 260288, 260289, 260290, 260291, 260292) c6(260293, 260294) c7(bL, W, pX, fc, hl, cz, Ja, dG, v, cP, ei, Hu); #c1(C1QTNF1) c2(XP_006721726) c3(complement C1q tumor necrosis factor-related protein 1 isoform X2) c4(294245) c5( ) c6( ) c7(bL, W, pX, fc, hl, cz, Ja, dG, v, cP, ei, Hu); #c1(C1QTNF1) c2(XP_006721727) c3(complement C1q tumor necrosis factor-related protein 1 isoform X4) c4(294246, 294247, 294248, 294249) c5(294250) c6( ) c7(bL, W, pX, fc, hl, cz, Ja, dG, v, cP, ei, Hu); #c1(C1QTNF1) c2(XP_006721729) c3(complement C1q tumor necrosis factor-related protein 1 isoform X3) c4( ) c5(294251, 294252, 294253, 294254, 294255, 294256, 294257, 294258, 294259, 294260, 294261, 294262, 294263, 294264, 294265, 294266, 294267) c6(294268, 294269, 294270, 294271) c7(bL, W, pX, fc, hl, cz, Ja, dG, v, cP, ei, Hu); #c1(C1QTNF3) c2(NP_112207) c3(complement C1q tumor necrosis factor-related protein 3 isoform a precursor) c4(220789, 220790) c5(220791, 220792, 220793, 220794, 220795, 220796, 220797, 220798, 220799, 220800, 220801, 220802, 220803, 220804, 220805, 220806, 220807, 220808, 220809, 220810, 220811, 220812, 220813) c6(220814, 220815, 220816, 220817) c7(bL, bM, kl, aZ, cz, v); #c1(C1QTNF3) c2(NP_852100) c3(complement C1q tumor necrosis factor-related protein 3 isoform b precursor) c4(254839, 254840) c5(254841, 254842, 254843, 254844, 254845, 254846, 254847, 254848) c6(254849, 254850) c7(bL, bM, kl, aZ, cz, v); #c1(C1QTNF5) c2(NP_001265360) c3(complement C1q tumor necrosis factor-related protein 5 precursor) c4(98367) c5(98368, 98369, 98370, 98371, 98372, 98373, 98374, 98375, 98376) c6( ) c7(W, lR, nb, axf, om, aqV, nw, v, nC, aqY); #c1(C1QTNF6) c2(NP_872292) c3 (complement C1q tumor necrosis factor-related protein 6) c4(256011) c5(256012, 256013, 256014, 256015, 256016, 256017, 256018, 256019, 256020, 256021, 256022, 256023, 256024, 256025) c6(256026, 256027, 256028, 256029) c7(bL, A, Y, hD, v, qj, gT, cd, y); #c1 (C1QTNF6) c2(XP_011528159) c3(complement C1q tumor necrosis factor-related protein 6 isoform X1) c4(323276) c5(323277, 323278, 323279, 323280, 323281, 323282, 323283, 323284) c6(323285, 323286) c7(bL, A, Y, HD, v, qj, gT, cd, y); #c1(C1QTNF7) c2(NP_001128642) c3(complement C1q tumor necrosis factor-related protein 7 isoform a precursor) c4(50426, 50427) c5(50428, 50429, 50430, 50431, 50432, 50433, 50434, 50435, 50436, 50437, 50438) c6(50439, 50440) c7(Gp, iW, v, EQ); #c1(C1QTNF7) c2(NP_114117) c3(complement C1q tumor necrosis factor-related protein 7 isoform b precursor) c4(222201) c5(222202, 222203, 222204, 222205, 222206, 222207, 222208, 222209, 222210, 222211, 222212, 222213, 222214, 222215, 222216, 222217, 222218, 222219, 222220) c6(222221, 222222, 222223, 222224, 222225, 222226) c7(Gp, iW, v, EQ); #c1(C1QTNF9B-AS1) c2(NP_001014442) c3(prostate collagen triple helix protein isoform 1) c4( ) c5(25922, 25923, 25924, 25925, 25926) c6( ) c7(ae, ad); #c1(C1QTNF9B-AS1) c2(NP_001129288) c3 (prostate collagen triple helix protein isoform 2) c4(51362) c5( ) c6( ) c7(ae, ad); #c1(C1R) c2(NP_001724) c3(complement C1r subcomponent precursor) c4(120530, 120531, 120532) c5(120533, 120534, 120535, 120536, 120537, 120538, 120539, 120540, 120541, 120542, 120543, 120544, 120545, 120546, 120547, 120548, 120549, 120550) c6( ) c7(dP, WP, af); #c1(C1RL) c2(NP_001284572) c3(complement C1r subcomponent-like protein isoform 4 precursor) c4( ) c5(112061) c6( ) c7(iW); #c1(C1RL) c2(NP_057630) c3(complement C1r subcomponent-like protein isoform 1 precursor) c4(191980, 191981) c5(191982, 191983, 191984, 191985, 191986, 191987, 191988, 191989, 191990, 191991, 191992, 191993) c6( ) c7(iW); #c1(CIS) c2(XP_005253817) c3(complement Cis subcomponent isoform X1) c4(272695, 272696, 272697, 272698, 272699) c5(272700, 272701, 272702, 272703, 272704, 272705, 272706, 272707, 272708, 272709, 272710, 272711, 272712, 272713, 272714, 272715, 272716, 272717, 272718, 272719, 272720, 272721, 272722, 272723, 272724, 272725, 272726, 272727, 272728, 272729, 272730, 272731, 272732, 272733, 272734, 272735, 272736, 272737, 272738, 272739, 272740, 272741, 272742, 272743) c6( ) c7(axh, iW, RJ, eZ, bh, axg); #c1(C2) c2(NP_000054) c3(complement CZ isoform I preproprotein) c4(1032, 1033, 1034) c5(1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064) c6( ) c7(bL, dD, axi, fR, iW, aj, j, eq, V, ad, od, rw, agD, h, ey, aaW, acd, lM, af, mu, hM, A, lR, ZC, cr, eZ, fe, da, dq, as, Uk, D, Bv, W, arj, RJ, axj, Br, GF, lf, B, y); #c1(C2) c2(NP_001139375) c3(complement C2 isoform 2 precursor) c4(59227, 59228, 59229, 59230) c5(59231, 59232, 59233, 59234, 59235, 59236, 59237, 59238, 59239, 59240, 59241, 59242, 59243, 59244, 59245, 59246, 59247, 59248, 59249, 59250, 59251, 59252, 59253, 59254, 59255, 59256, 59257, 59258, 59259, 59260, 59261, 59262, 59263, 59264, 59265, 59266, 59267, 59268, 59269, 59270, 59271, 59272, 59273, 59274, 59275, 59276, 59277, 59278, 59279, 59280) c6( ) c7(bL, dD, axi, fR, iW, aj, j, eq, V, ad, od, rw, agD, h, ey, aaW, acd, lM, af, mu, hM, A, lR, ZC, cr, eZ, fe, da, dq, as, Uk, D, Bv, W, arj, RJ, axj, Br, GF, lf, B, y); #c1(CZ) c2(NP_001171534) c3(complement CZ isoform 3) c4(70471, 70472, 70473, 70474) c5(70475, 70476, 70477, 70478, 70479, 70480, 70481, 70482, 70483, 70484, 70485, 70486, 70487, 70488, 70489, 70490, 70491, 70492, 70493, 70494, 70495, 70496, 70497, 70498, 70499, 70500, 70501, 70502, 70503, 70504, 70505, 70506, 70507, 70508, 70509, 70510, 70511, 70512, 70513, 70514, 70515, 70516, 70517, 70518, 70519, 70520, 70521, 70522, 70523, 70524, 70525, 70526, 70527, 70528, 70529, 70530, 70531, 70532, 70533, 70534, 70535, 70536, 70537, 70538, 70539) c6( ) c7(bL, dD, axi, fR, iW, aj, j, eq, V, ad, od, rw, agD, h, ey, aaW, acd, lM, af, mu, hM, A, lR, ZC, cr, eZ, fe, da, dq, as, Uk, D, Bv, W, arj, RJ, axj, Br, GF, lf, B, y); #c1(C2) c2(NP_001269386) c3(complement CZ isoform 4) c4(101403, 101404, 101405) c5(101406, 101407, 101408, 101409, 101410, 101411, 101412, 101413, 101414, 101415, 101416, 101417, 101418, 101419, 101420, 101421, 101422, 101423, 101424, 101425, 101426, 101427, 101428, 101429, 101430, 101431, 101432, 101433, 101434, 101435, 101436, 101437, 101438, 101439, 101440, 101441, 101442, 101443, 101444, 101445, 101446, 101447, 101448, 101449, 101450, 101451, 101452, 101453, 101454, 101455, 101456, 101457, 101458, 101459, 101460, 101461, 101462, 101463, 101464, 101465, 101466, 101467, 101468, 101469, 101470, 101471, 101472, 101473, 101474, 101475, 101476, 101477, 101478, 101479, 101480, 101481) c6( ) c7(bL, dD, axi, fR, iW, aj, j, eq, V, ad, od, rw, agD, h, ey, aaW, acd, lM, af, mu, hM, A, lR, ZC, cr, eZ, fe, da, dq, as, Uk, D, Bv, W, arj, RJ, axj, Br, GF, lf, B, y); #c1(C2) c2(NP_001269388) c3(complement C2 isoform 6 precursor) c4( ) c5(101482, 101483, 101484, 101485, 101486, 101487, 101488, 101489, 101490, 101491, 101492, 101493, 101494, 101495, 101496, 101497, 101498, 101499, 101500, 101501, 101502, 101503, 101504, 101505, 101506, 101507, 101508, 101509, 101510, 101511, 101512, 101513, 101514, 101515, 101516, 101517, 101518, 101519, 101520, 101521, 101522, 101523, 101524, 101525, 101526, 101527, 101528, 101529, 101530, 101531, 101532, 101533, 101534, 101535, 101536, 101537, 101538, 101539, 101540, 101541, 101542, 101543, 101544, 101545, 101546, 101547, 101548, 101549, 101550, 101551, 101552, 101553) c6( ) c7(bL, dD, axi, fR, iW, aj, j, eq, V, ad, od, rw, agD, h, ey, aaW, acd, lM, af, mu, hM, A, lR, ZC, cr, eZ, fe, da, dq, as, Uk, D, Bv, W, arj, RJ, axj, Br, GF, lf, B, y); #c1(C20orf194) c2(NP_001009984) c3(uncharacterized protein C20orf194) c4(23776) c5(23777, 23778, 23779, 23780, 23781, 23782) c6( ) c7(F, rf); #c1(C20orf194) c2(XP_005260741) c3(uncharacterized protein C20orf194 isoform X1) c4( ) c5(276824, 276825) c6( ) c7(F, rf); #c1(C20orf194) c2(XP_005260743) c3(uncharacterized protein C20orf194 isoform X4) c4(276826) c5(276827, 276828, 276829, 276830, 276831) c6( ) c7(F, rf); #c1(C20orf196) c2(NP_001290406) c3(uncharacterized protein C20orf196 isoform 1) c4( ) c5(115345, 115346, 115347, 115348, 115349, 115350, 115351, 115352, 115353, 115354, 115355, 115356, 115357, 115358, 115359) c6( ) c7(aaw, dY); #c1(C20orf196) c2(NP_001290408) c3(uncharacterized protein C20orf196 isoform 3) c4(115360) c5(115361, 115362, 115363, 115364, 115365) c6( ) c7(aaw, dY); #c1(C20orf196) c2(XP_006723612) c3(uncharacterized protein C20orf196 isoform X2) c4(295248, 295249, 295250, 295251, 295252) c5(295253, 295254, 295255, 295256, 295257, 295258, 295259) c6( ) c7(aaw, dY): #c1(C20orf196) c2(XP_011527479) c3(uncharacterized protein C20orf196 isoform X1) c4( ) c5(322276, 322277, 322278) c6( ) c7(aaw, dY); #c1(C20orf196) c2(XP_011527482) c3(uncharacterized protein C20orf196 isoform X5) c4( ) c5(322279) c6( ) c7(aaw, dY); #c1(C20orf196) c2(XP_011527484) c3(uncharacterized protein C20orf196 isoform X7) c4( ) c5(322280, 322281, 322282, 322283, 322284) c6( ) c7(aaw, dY); #c1(C20orf27) c2(NP_001034229) c3(UPF0687 protein C20orf27 isoform 1) c4(31613) c5(31614, 31615, 31616, 31617, 31618, 31619) c6( ) c7(j); #c1(C20orf27) c2(NP_001245359) c3(UPF0687 protein C20orf27 isoform 2) c4(93241) c5(93242, 93243, 93244, 93245, 93246, 93247, 93248, 93249, 93250, 93251) c6( ) c7(j); #c1(C20orf27) c2(XP_011527568) c3(UPF0687 protein C20orf27 isoform X1) c4(322416, 322417) c5(322418, 322419, 322420, 322421, 322422, 322423, 322424, 322425, 322426, 322427, 322428, 322429) c6( ) c7(j); #c1(C20orf85) c2(NP_848551) c3 (uncharacterized protein C20orf85) c4(253827) c5(253828, 253829, 253830, 253831, 253832, 253833) c6( ) c7(av, DA, eM, ff, en, bz, iu); #c1(C21orf2) c2(NP_001258369) c3(nuclear encoded mitochondrial protein C21orf2 isoform 2) c4(96018) c5(96019, 96020, 96021, 96022, 96023) c6( ) c7(cc, OM, f, Bh, af, wT); #c1(C21orf2) c2(NP_001258370) c3(nuclear encoded mitochondrial protein C21orf2 isoform 3) c4(96024) c5(96025, 96026, 96027, 96028, 96029, 96030) c6( ) c7(cc, OM, f, Bh, af, wT); #c1(C21orf2) c2(NP_004919) c3(nuclear encoded mitochondrial protein C21orf2 isoform 1) c4(150786) c5(150787, 150788, 150789, 150790, 150791, 150792, 150793) c6( ) c7(cc, OM, f, Bh, af, wT); #c1(C21orf33) c2(NP_004640) c3(ESI protein homolog, mitochondrial isoform 1a precursor) c4(148432) c5(148433, 148434, 148435, 148436, 148437, 148438) c6( ) c7(Fv, T, os, fM, aC, iB, aA, bB, n, axk, oZ, ci, il, q, ad, eA, ip, iC, bv, av, ft, x, aE, ahl, KH, aT, bl, Tj, fb, ae, Qe, jf, axl, A, bf, s, iU, As, aW, Y, eH, cr, r, nY, hN, aq, aF, g, cl, as, t, f, bz, eF, cm, iV, ck, iO, c, bE, m, be, bt, B); #c1(C21orf33) c2(NP_937798) c3(ESI protein homolog, mitochondrial isoform 1b precursor) c4(259125) c5(259126, 259127, 259128, 259129, 259130, 259131) c6( ) c7(Fv, T, os, fM, aC, iB, aA, bB, n, axk, oZ, ci, il, q, ad, eA, ip, iC, bv, av, ft, x, aE, ahL, KH, aT, bl, Tj, fb, ae, Qe, jf, axl, A, bf, s, iU, As, aW, Y, eH, cr, r, nY, hN, aq, aF, g, cl, as, t, f, bz, eF, cm, iV, ck, iO, c, bE, m, be, bt, B); #c1(C21orf59) c2(NP_067077) c3(UPF0769 protein C21orf59) c4(208917, 208918, 208919) c5(208920, 208921, 208922, 208923, 208924, 208925, 208926, 208927, 208928, 208929, 208930, 208931, 208932, 208933, 208934, 208935, 208936, 208937) c6( ) c7(Pf, axm, MG); #c1(C21orf62) c2(XP_011545334) c3(uncharacterized protein C21orf62 isoform X1) c4(344097) c5(344098, 344099, 344100, 344101, 344102, 344103, 344104, 344105, 344106, 344107, 344108, 344109, 344110, 344111, 344112, 344113, 344114, 344115, 344116) c6( ) c7(RI); #c1(C21orf91) c2(NP_001093890) c3(protein EURL homolog isoform 1) c4(40504, 40505) c5(40506, 40507, 40508, 40509, 40510, 40511) c6( ) c7(ey); #c1(C21orf91) c2(NP_001093891) c3(protein EURL homolog isoform 3) c4( ) c5(40512, 40513, 40514, 40515, 40516) c6( ) c7(ey); #c1(C21orf91) c2(NP_059143) c3(protein EURL homolog isoform 2) c4(193262, 193263) c5(193264, 193265, 193266, 193267, 193268, 193269, 193270, 193271, 193272, 193273, 193274, 193275, 193276) c6( ) c7(ey); #c1(C22orf29) c2(NP_078903) c3(protein Bop) c4(216003) c5(216004, 216005, 216006, 216007, 216008, 216009, 216010, 216011, 216012, 216013, 216014, 216015, 216016, 216017) c6( ) c7(ae, T, fM, asp, py, aW, Y, jh, cj, aT, fd, kp, fr, K, bz, iu, ad); #c1(C2CD3) c2(NP_001273506) c3(C2 domain-containing protein 3 isoform 1) c4(105095, 105096, 105097) c5(105098, 105099, 105100, 105101, 105102, 105103) c6( ) c7(ahD); #c1(C2CD3) c2(NP_056346) c3(C2 domain-containing protein 3 isoform 2) c4(187186) c5(187187, 187188, 187189, 187190, 187191, 187192, 187193, 187194, 187195, 187196, 187197, 187198, 187199) c6( ) c7(ahD); #c1(C2CD3) c2(XP_011543209) c3(CZ domain-containing protein 3 isoform X7) c4( ) c5(342019, 342020, 342021, 342022, 342023) c6( ) c7(ahD); #c1(C2CD4A) c2(NP_997205) c3(CZ calcium-dependent domain-containing protein 4A) c4(265173) c5(265174, 265175, 265176, 265177, 265178, 265179) c6( ) c7(bl, aj, J, c); #c1(C2CD4B) c2(NP_001007596) c3(C2 calcium-dependent domain-containing protein 48) c4(22848) c5(22849, 22850, 22851, 22852, 22853, 22854) c6( ) c7(bL, aj, e, J); #c1(C2CD5) c2(NP_001273104) c3(C2 domain-containing protein 5 isoform b) c4(104321) c5(104322, 104323, 104324, 104325, 104326, 104327) c6( ) c7(e); #c1(C2CD5) c2(NP_055617) c3(C2 domain-containing protein 5 isoform e) c4(182308) c5(182309, 182310, 182311, 182312, 182313, 182314) c6( ) c7(e); #c1(C2orf16) c2(NP_115642) c3(uncharacterized protein C2orf16) c4(223412) c5(223413, 223414, 223415, 223416, 223417, 223418, 223419, 223420, 223421) c6( ) c7(om); #c1(C2orf40) c2(NP_115787) c3(augurin precursor) c4(223903) c5(223904, 223905, 223906, 223907, 223908, 223909) c6( ) c7(T, aW, ff, ht, aT, eA, en, hu, it, q); #c1(C2orf43) c2(NP_001269649) c3(UPF0554 protein C2orf43 isoform c) c4(102406) c5(102407, 102408, 102409, 102410, 102411, 102412) c6( ) c7(om, ae, j, ad); #c1(C2orf43) c2(NP_001269650) c3(UPF0554 protein C2orf43 isoform d) c4( ) c5(102413, 102414, 102415, 102416, 102417) c6( ) c7(om, ae, j, ad); #c1(C2orf43) c2(NP_001269652) c3(UPF0554 protein C2orf43 isoform e) c4(102418) c5(102419, 102420, 102421, 102422, 102423, 102424) c6( ) c7(om, ae, j, ad); #c1(C2orf43) c2(NP_068744) c3(UPF0554 protein C2orf43 isoform a) c4(210148) c5(210149, 210150, 210151, 210152, 210153, 210154, 210155) c6( ) c7(om, ae, j, ad); #c1(C2orf43) c2(XP_011531316) c3(UPF0554 protein C2orf43 isoform X1) c4(327312) c5( ) c6( ) c7(om, ae, j, ad); #c1(C2orf47) c2(NP_078796) c3(uncharacterized protein C2orf47, mitochondrial precursor) c4(215516) c5(215517, 215518, 215519, 215520, 215521, 215522, 215523, 215524, 215525, 215526) c6( ) c7(k); #c1(C2orf57) c2(NP_689827) c3(uncharacterized protein C2orf57) c4(244051) c5(244052, 244053, 244054, 244055, 244056, 244057) c6( ) c7(fq); #c1(C2orf61) c2(NP_001157033) c3(uncharacterized protein C2orf61 isoform 1) c4(62054) c5(62055, 62056, 62057, 62058, 62059, 62060) c6( ) c7(fq); #c1(C2orf61) c2(NP_775920) c3 (uncharacterized protein C2orf61 isoform 2) c4(250704) c5(250705, 250706, 250707, 250708, 250709, 250710) c6( ) c7(fq); #c1(C3) c2(NP_000055) c3(complement C3 precursor) c4(1065) c5(1066, 1067, 1068, 1069, 1070, 1071) c6( ) c7(bM, ea, bB, ci, x, aE, u, Bc, Ds, dd, aW, axu, md, wf, awv, da, vs, as, eE, lk, axp, uC, Uk, iA, nw, Dt, k, m, qR, p, dD, My, aw, n, fr, uE, ai, ql, h, lG, ey, Um, A, sp, fe, cM, kT, W, w, Fi, afc, mg, JM, uz, eo, fL, bL, Dw, axt, dk, v, j, C, so, il, V, o, aT, agD, axn, an, ae, af, eu, kl, bf, oV, t, axo, ac, c, WP, hc, of, DW, JH, fD, dM, iW, uu, eq, mC, dP, l, axq, akJ, JK, ul, axr, cr, eZ, Z, DF, dR, Hw, gk, axs, nb, ag, do, arj, ei, GF, y, vt, fh); #c1 (C3ARI) c2(NP_004045) c3(C3a anaphylatoxin chemotactic receptor) c4(143123, 143124) c5(143125, 143126, 143127, 143128, 143129, 143130, 143131, 143132, 143133, 143134, 143135, 143136) c6( ) c7(bM, W, sp, iW, at, lG, v, JH, ey, C, fD, mF, V, aa); #c1(C3orf17) c2(NP_056227) c3(uncharacterized protein C3orf17) c4(186599, 186600) c5(186601, 186602, 186603, 186604, 186605, 186606, 186607) c6( ) c7(e, af); #c1(C3orf17) c2(XP_005247330) c3(uncharacterized protein C3orf17 isoform X1) c4(267799) c5(267800, 267801, 267802, 267803, 267804, 267805) c6( ) c7(e, af); #c1(C3orf17) c2(XP_005247333) c3(uncharacterized protein C3orf17 isoform X2) c4( ) c5(267806, 267807, 267808, 267809, 267810) c6( ) c7(e, af); #c1(C3orf17) c2(XP_005247335) c3(uncharacterized protein C3orf17 isoform X3) c4(267811) c5( ) c6( ) c7(e, af); #c1(C3orf17) c2(XP_011510940) c3(uncharacterized protein C3orf17 isoform X4) c4( ) c5(301409, 301410, 301411, 301412, 301413, 301414) c6( ) c7(e, af); #c1(C3orf18) c2(NP_001165212) c3 (uncharacterized protein C3orf18 isoform 1) c4(67539) c5(67540, 67541, 67542, 67543, 67544, 67545, 67546, 67547, 67548, 67549, 67550) c6( ) c7(h); #c1(C3orf18) c2(XP_011532091) c3 (uncharacterized protein C3orf18 isoform X4) c4( ) c5(328140, 328141, 328142, 328143) c6( ) c7(h); #c1(C3orf18) c2(XP_011532092) c3(uncharacterized protein C3orf18 isoform X1) c4(328144) c5(328145, 328146, 328147, 328148, 328149) c6( ) c7(h); #c1(C3orf20) c2(NP_001171887) c3(uncharacterized protein C3orf20 isoform 2) c4( ) c5(71958, 71959, 71960, 71961, 71962, 71963, 71964, 71965) c6( ) c7(an); #c1(C3orf20) c2(NP_115513) c3(uncharacterized protein C3orf20 isoform 1) c4(223018, 223019) c5(223020, 223021, 223022, 223023, 223024, 223025, 223026, 223027) c6( ) c7(an); #c1(C3orf35) c2(NP_848029) c3(AP20 region protein 1 isoform B) c4(253716, 253717) c5(253718, 253719, 253720, 253721, 253722, 253723, 253724, 253725, 253726, 253727, 253728, 253729) c6( ) c7(T, ft, aW, aT, n, iK); #c1(C3orf35) c2(NP_848032) c3(AP20 region protein 1 isoform E) c4(253730) c5(253731, 253732, 253733, 253734, 253735, 253736, 253737, 253738, 253739, 253740, 253741, 253742, 253743, 253744) c6( ) c7(T, ft, aW, aT, n, iK); #c1(C3orf56) c2(NP_001007535) c3(putative uncharacterized protein C3orf56) c4(22781) c5(22782, 22783, 22784, 22785, 22786, 22787) c6( ) c7(af); #c1(C3orf58) c2(NP_001127942) c3(deleted in autism protein 1 isoform b) c4(49519) c5(49520, 49521, 49522, 49523, 49524, 49525) c6( ) c7(ck, T, VX, Y, axv, aw, cy, nA, ad, bN); #c1(C3orf58) c2(NP_775823) c3(deleted in autism protein 1 isoform a precursor) c4(250439) c5(250440, 250441, 250442, 250443, 250444, 250445) c6( ) c7(ck, T, VX, Y, axv, aw, cy, nA, ad, bN); #c1(C3orf67) c2(NP_940865) c3(uncharacterized protein C3orf67) c4(259983) c5( ) c6( ) c7(gZ, af, ac); #c1(C3orf67) c2(XP_006713076) c3(uncharacterized protein C3orf67 isoform X1) c4(287017) c5(287018) c6( ) c7(gZ, af, ac); #c1(C3orf67) c2(XP_011531755) c3(uncharacterized protein C3orf67 isoform X6) c4( ) c5(327838) c6( ) c7(gZ, af, ac); #c1(C3orf67) c2(XP_011531756) c3(uncharacterized protein C3orf67 isoform X7) c4(327839) c5( ) c6( ) c7(gZ, af, ac); #c1(C3orf67) c2(XP_011531761) c3(uncharacterized protein C3orf67 isoform X10) c4( ) c5(327840, 327841, 327842, 327843, 327844, 327845) c6( ) c7(gZ, af, ac); #c1(C3orf67) c2(XP_011531765) c3(uncharacterized protein C3orf67 isoform X13) c4( ) c5(327846, 327847, 327848, 327849, 327850) c6( ) c7(gZ, af, ac); #c1(C3orf67) c2(XP_011531766) c3 (uncharacterized protein C3orf67 isoform X9) c4( ) c5(327851, 327852, 327853, 327854, 327855) c6( ) c7(gZ, af, ac); #c1(C3orf79) c2(NP_001094807) c3(putative uncharacterized protein C3orf79) c4(40751) c5(40752, 40753, 40754, 40755, 40756, 40757) c6( ) c7(aeP, bM); #c1(C4A) c2(NP_001239133) c3(complement C4-A isoform 2 preproprotein) c4(88265, 88266, 88267, 88268, 88269, 88270, 88271) c5(88272, 88273, 88274, 88275, 88276, 88277, 88278, 88279, 88280, 88281, 88282, 88283, 88284, 88285, 88286, 88287, 88288, 88289, 88290, 88291, 88292, 88293, 88294, 88295, 88296, 88297, 88298, 88299, 88300, 88301, 88302, 88303, 88304, 88305, 88306, 88307, 88308, 88309, 88310, 88311, 88312, 88313, 88314, 88315, 88316, 88317, 88318, 88319, 88320, 88321, 88322, 88323, 88324, 88325, 88326, 88327, 88328, 88329, 88330, 88331, 88332, 88333, 88334, 88335, 88336, 88337, 88338, 88339, 88340, 88341, 88342, 88343, 88344) c6(88345, 88346, 88347, 88348) c7(bL, bM, fR, dM, e, al, aw, fN, iW, cL, aj, qj, bh, mC, J, vV, axy, adX, hD, lM, af, bc, hm, vl, wC, A, axz, oV, lC, cr, eZ, aq, axx, axw, EZ, cM, fq, bN, lY, ac, adZ, hc, Ny, at, Vw, oh, JH, fL, y, B, pC); #c1(C4A) c2(NP_009224) c3(complement C4-A isoform 1 preproprotein) c4(171577, 171578, 171579, 171580) c5(171581, 171582, 171583, 171584, 171585, 171586, 171587, 171588, 171589, 171590, 171591, 171592, 171593, 171594, 171595, 171596, 171597, 171598, 171599, 171600, 171601, 171602, 171603, 171604, 171605, 171606) c6( ) c7(bL, bM, fR, dM, e, al, aw, fN, iW, cL, aj, qj, bh, mC, J, vV, axy, adX, HD, lM, af, bc, hm, vl, wC, A, axz, oV, lC, cr, eZ, aq, axx, axw, EZ, cM, fq, bN, lY, ac, adZ, hc, Ny, at, Vw, oh, JH, fL, y, B, pC); #c1(C4B) c2(NP_001002029) c3(complement C4-B preproprotein) c4(19377, 19378, 19379, 19380, 19381, 19382) c5(19383, 19384, 19385, 19386, 19387, 19388, 19389, 19390, 19391, 19392, 19393, 19394, 19395, 19396, 19397, 19398, 19399) c6( ) c7(bL, e, aw, fN, aj, qj, bh, ai, mC, J, vV, axy, adX, HD, DX, af, bc, hm, vl, A, axz, lC, cr, eZ, aq, axx, axw, EZ, cM, bN, lY, ac, adZ, hc, Ny, oh, JH, fL, y, B, pC); #c1(C4B_2) c2(NP_001229752) c3(complement C4-B-like preproprotein) c4(85060, 85061) c5(85062, 85063, 85064, 85065, 85066, 85067, 85068, 85069, 85070, 85071, 85072, 85073, 85074, 85075, 85076, 85077, 85078, 85079, 85080, 85081, 85082, 85083, 85084, 85085, 85086, 85087, 85088, 85089, 85090, 85091, 85092, 85093, 85094, 85095, 85096, 85097, 85098, 85099, 85100, 85101, 85102, 85103, 85104, 85105) c6(85106, 85107, 85108, 85109, 85110, 85111) c7(bL, e, aw, fN, aj, qj, bh, mC, J, vV, axy, adX, hD, af, bc, hm, vl, A, axz, lC, cr, eZ, aq, axx, axw, EZ, bN, lY, ac, adZ, hc, Ny, oh, fl, y, B, pC); #c1(C4BPA) c2(XP_005273308) c3(C4b-binding protein alpha chain isoform X1) c4(284434, 284435) c5(284436, 284437, 284438, 284439, 284440, 284441, 284442, 284443, 284444, 284445, 284446, 284447) c6( ) c7(ka, dt, kh, iW, axA, jX, hF, q, jY, hX, av, anj, ec, hb, ql, ds, aT, h, hH, aW, BG, Xk, kk, dr, dS, ck, jV, W, eM, ki, kq, jQ, it); #c1(C4BPB) c2(NP_001017365) c3(C4b-binding protein beta chain isoform 1 precursor) c4(26259, 26260) c5(26261, 26262, 26263, 26264, 26265, 26266, 26267, 26268, 26269, 26270, 26271, 26272, 26273, 26274, 26275, 26276, 26277, 26278, 26279, 26280) c6( ) c7(W, eM, ec, iW, YG, hH, dr); #c1(C4BPB) c2(NP_001017366) c3(C4b-binding protein beta chain isoform 2 precursor) c4(26281) c5(26282, 26283, 26284, 26285, 26286, 26287) c6( ) c7(W, eM, ec, iW, YG, hH, dr); #c1(C4BPB) c2(XP_005273312) c3(C4b-binding protein beta chain isoform X2) c4(284448) c5(284449, 284450, 284451, 284452, 284453, 284454, 284455, 284456, 284457, 284458, 284459, 284460, 284461, 284462, 284463, 284464, 284465, 284466, 284467, 284468, 284469, 284470, 284471, 284472, 284473, 284474, 284475, 284476, 284477, 284478) c6( ) c7(W, eM, ec, iW, YG, hH, dr); #c1(C4orf22) c2(NP_001193926) c3 (uncharacterized protein C4orf22 isoform 1) c4(83188) c5(83189, 83190, 83191, 83192, 83193, 83194) c6( ) c7(oe, pN); #c1(C4orf22) c2(NP_689983) c3(uncharacterized protein C4orf22 isoform 2) c4(244659) c5(244660, 244661, 244662, 244663, 244664, 244665) c6( ) c7(oe, pN); #c1(C4orf26) c2(NP_001193910) c3(uncharacterized protein C4orf26 isoform 1 precursor) c4(83143) c5(83144, 83145, 83146, 83147, 83148, 83149, 83150, 83151, 83152, 83153) c6(83154, 83155) c7(Ud, axB); #c1(C4orf26) c2(NP_848592) c3(uncharacterized protein C4orf26 isoform 2 precursor) c4(253917) c5(253918, 253919, 253920, 253921, 253922, 253923, 253924) c6(253925, 253926) c7(Ud, axB); #c1(C4orf27) c2(NP_060337) c3(UPF0609 protein C4orf27) c4(195458) c5(195459, 195460, 195461, 195462, 195463, 195464, 195465) c6( ) c7(ey); #c1(C4orf32) c2(NP_689613) c3(uncharacterized protein C4orf32) c4(243260) c5(243261, 243262, 243263, 243264, 243265, 243266) c6( ) c7(bL); #c1(C4orf33) c2(NP_001093253) c3(UPF0462 protein C4orf33) c4(40177) c5(40178, 40179, 40180, 40181, 40182, 40183, 40184, 40185, 40186, 40187) c6( ) c7(v, oe, j, ac, cz); #c1(C4orf33) c2(XP_011529896) c3(UPF0462 protein C4orf33 isoform X2) c4(325664) c5(325665, 325666, 325667, 325668, 325669, 325670) c6( ) c7(v, oe, j, ac, cz); #c1(C4orf36) c2(XP_011529917) c3(uncharacterized protein C4orf36 isoform X1) c4(325677, 325678, 325679) c5(325680, 325681, 325682, 325683, 325684, 325685, 325686, 325687, 325688, 325689, 325690, 325691, 325692, 325693, 325694, 325695, 325696, 325697, 325698, 325699, 325700, 325701) c6( ) c7(c); #c1(C4orf48) c2(NP_001135408) c3(neuropeptide-like protein C4orf48 isoform 2 precursor) c4( ) c5(52868, 52869, 52870, 52871, 52872, 52873, 52874) c6(52875) c7(axC); #c1(C4orf48) c2(NP_001161715) c3(neuropeptide-like protein C4orf48 isoform 1) c4(65863, 65864) c5(65865, 65866, 65867, 65868, 65869, 65870, 65871, 65872, 65873, 65874, 65875, 65876, 65877, 65878, 65879, 65880) c6(65881, 65882, 65883, 65884) c7(axC); #c1(C4orf48) c2(XP_011511770) c3(neuropeptide-like protein C4orf48 isoform X2) c4(302470) c5( ) c6( ) c7(axC); #c1(C4orf51) c2(XP_011530499) c3(uncharacterized protein C4orf51 isoform X1) c4(326386, 326387) c5(326388, 326389, 326390, 326391, 326392, 326393, 326394, 326395, 326396, 326397, 326398, 326399) c6( ) c7(ac); #c1(C5) c2(NP_001726) c3(complement C5 preproprotein) c4(120551) c5(120552, 120553, 120554, 120555, 120556, 120557) c6( ) c7(ng, gV, bL, ajy, dD, bM, Dw, iW, al, fF, cL, j, eq, C, Jx, ai, dP, ke, F, Bc, axE, lG, Ds, cy, JK, K, ey, ji, db, eu, ul, A, oV, sp, DN, fO, axu, eZ, lo, ajz, cM, axD, OW, W, ac, afc, mk, Hb, y, qR, B); #c1(C5ARI) c2(NP_001727) c3(CSa anaphylatoxin chemotactic receptor 1) c4(120558) c5(120559, 120560, 120561, 120562, 120563, 120564) c6( ) c7(ng, gV, T, bM, dk, iW, axF, dG, v, kO, C, yV, Jx, ai, ke, F, afn, axE, lG, cy, ey, K, ji, H, af, aa, lR, fO, ih, aq, lo, ajz, wA, bz, axG, W, c, eM, wl, vy, at, dy, Hb, fD, y, B, L); #c1(C5AR2) c2(NP_001258679) c3(C5a anaphylatoxin chemotactic receptor 2) c4(96317) c5(96318, 96319, 96320, 96321, 96322, 96323, 96324, 96325, 96326, 96327) c6( ) c7(bL, Fi, v, j, ji, dQ); #c1(C5orf22) c2(NP_060826) c3(UPF0489 protein C5orf22) c4(198012) c5(198013, 198014, 198015, 198016, 198017, 198018, 198019) c6( ) c7(c); #c1(C5orf22) c2(XP_006714542) c3(UPF0489 protein C5orf22 isoform X2) c4(287901, 287902) c5( ) c6( ) c7(c); #c1(C5orf22) c2(XP_006714543) c3(UPF0489 protein C5orf22 isoform X3) c4( ) c5(287903, 287904, 287905, 287906, 287907, 287908, 287909, 287910, 287911, 287912, 287913) c6( ) c7(c); #c1(C5orf30) c2(NP_149988) c3(UNC119-binding protein C5orf30) c4(227721) c5(227722, 227723, 227724, 227725, 227726, 227727, 227728, 227729, 227730, 227731, 227732, 227733, 227734, 227735, 227736, 227737, 227738) c6( ) c7(y); #c1(C5orf34) c2(NP_940968) c3(uncharacterized protein C5orf34) c4(260209) c5(260210, 260211, 260212, 260213, 260214, 260215) c6( ) c7(j); #c1(C5orf38) c2(NP_001281266) c3(protein CEI isoform 2 precursor) c4(1/1632) c5(111633, 111634, 111635, 111636, 111637, (11638, 111639) c6(111640) c7(cd, ac, c); #c1(C5orf38) c2(NP_848664) c3(protein CEI isoform 1 precursor) c4(254147) c5(254148, 254149, 254150, 254151, 254152, 254153, 254154, 254155) c6(254156, 254157) c7(cd, ac, c); #c1(C5orf38) c2(XP_005248313) c3 (protein CEI isoform X1) c4( ) c5(268302) c6( ) c7(cd, ac, c); #c1(C5orf42) c2(NP_075561) c3(uncharacterized protein C5orf42) c4(213948) c5(213949, 213950, 213951, 213952, 213953, 213954, 213955, 213956, 213957, 213958) c6( ) c7(axl, axH); #c1(C5orf42) c2(XP_005248403) c3(uncharacterized protein C5orf42 isoform X3) c4(268380) c5(268381, 268382, 268383, 268384, 268385, 268386) c6( ) c7(axl, axH); #c1(C5orf42) c2(XP_005248406) c3(uncharacterized protein C5orf42 isoform X7) c4(268387) c5(268388, 268389, 268390, 268391, 268392, 268393) c6( ) c7(axl, axH); #c1(C5orf63) c2(NP_001157950) c3(glutaredoxin-like protein C5orf63 isoform 2) c4(62945) c5(62946, 62947, 62948, 62949, 62950, 62951) c6( ) c7(IB); #c1(C5orf63) c2(NP_001157951) c3(glutaredoxin-like protein C5orf63 isoform 1) c4(62952) c5(62953, 62954, 62955, 62956, 62957, 62958) c6( ) c7(IB); #c1(C6) c2(NP_000056) c3(complement component C6 precursor) c4(1072) c5( ) c6( ) c7(W, fR, up, eg, eR, hc, iW, axJ, lS, axk, C, wt, xe); #c1(C6) c2(XP_005248414) c3(complement component C6 isoform X5) c4( ) c5(268394, 268395, 268396, 268397, 268398, 268399, 268400, 268401, 268402, 268403) c6( ) c7(W, fR, up, eg, eR, hc, iW, axJ, lS, axk, C, wt, xe); #c1(C6orf10) c2(XP_011512542) c3(uncharacterized protein C6orf10 isoform X8) c4(303376, 303377, 303378, 303379, 303380) c5(303381, 303382, 303383, 303384, 303385, 303386, 303387, 303388, 303389, 303390, 303391, 303392, 303393, 303394, 303395, 303396, 303397, 303398, 303399, 303400, 303401, 303402) c6( ) c7(bL, bM, A, cr, cz, gZ, HD, cl, eZ, j, pt, V, bc, y); #c1(C6orf10) c2(XP_011512544) c3 (uncharacterized protein C6orf10 isoform X9) c4(303403) c5( ) c6( ) c7(bL, bM, A, cr, cz, gZ, HD, cl, eZ, j, pt, V, bc, y); #c1(C6orf10) c2(XP_011512545) c3(uncharacterized protein C6orf10 isoform X18) c4( ) c5(303404, 303405, 303406, 303407, 303408, 303409, 303410, 303411, 303412, 303413) c6( ) c7(bL, bM, A, cr, cz, gZ, hD, cL, eZ, j, pt, V, bc, y); #c1(C6orf10) c2(XP_011512548) c3(uncharacterized protein C6orf10 isoform X11) c4(303414, 303415) c5(303416, 303417, 303418, 303419, 303420, 303421, 303422, 303423, 303424, 303425, 303426, 303427) c6( ) c7(bL, bM, A, cr, cz, gZ, HD, cl, eZ, j, pt, V, bc, y); #c1(C6orf10) c2(XP_011545475) c3(uncharacterized protein C6orf10 isoform X25) c4( ) c5(344156, 344157, 344158, 344159, 344160, 344161) c6( ) c7(bL, bM, A, cr, cz, gZ, HD, cl, eZ, j, pt, V, bc, y); #c1(C6orf10) c2(XP_011545478) c3(uncharacterized protein C6orf10 isoform X3) c4(344162, 344163, 344164, 344165, 344166, 344167, 344168) c5(344169, 344170, 344171, 344172, 344173, 344174, 344175, 344/76, 344177, 344178, 344179, 344180, 344181, 344182, 344183, 344184, 344185, 344186, 344187, 344188, 344189, 344190, 344191, 344192, 344193, 344194, 344195, 344196, 344197, 344198, 344199, 344200, 344201, 344202, 344203, 344204, 344205, 344206, 344207, 344208, 344209, 344210, 344211, 344212, 344213, 344214, 344215, 344216, 344217) c6( ) c7(bL, bM, A, cr, cz, gZ, HD, cl, eZ, j, pt, V, bc, y); #c1(C6orf10) c2(XP_011546134) c3(uncharacterized protein C6orf10 isoform X10) c4(344849) c5(344850, 344851, 344852, 344853, 344854, 344855, 344856, 344857, 344858, 344859) c6( ) c7(bL, bM, A, cr, cz, gZ, hD, cL, eZ, j, pt, V, bc, y); #c1(C6orf10) c2(XP_011546684) c3(uncharacterized protein C6orf10 isoform X25) c4(345454) c5(345455, 345456, 345457, 345458, 345459, 345460, 345461, 345462, 345463, 345464, 345465, 345466, 345467, 345468, 345469, 345470) c6( ) c7(bL, bM, A, cr, cz, gZ, HD, cl, eZ, j, pt, V, bc, y); #c1(C6orf106) c2(NP_073595) c3(uncharacterized protein C6orf106 isoform b) c4(213022) c5(213023, 213024, 213025, 213026, 213027, 213028) c6( ) c7(xb); #c1(C6orf106) c2(NP_077270) c3(uncharacterized protein C6orf106 isoform a) c4(214840, 214841) c5(214842, 214843, 214844, 214845, 214846, 214847, 214848, 214849, 214850, 214851, 214852, 214853) c6( ) c7(xb); #c1(C6orf15) c2(NP_054789) c3(uncharacterized protein C6orf15 precursor) c4(177929, 177930, 177931) c5(177932, 177933, 177934, 177935, 177936, 177937, 177938, 177939, 177940, 177941, 177942, 177943, 177944, 177945, 177946, 177947, 177948, 177949, 177950, 177951, 177952, 177953, 177954, 177955, 177956, 177957, 177958, 177959, 177960, 177961, 177962, 177963, 177964, 177965, 177966) c6(177967, 177968, 177969, 177970, 177971, 177972, 177973, 177974) c7(hH, AW, y, eZ); #c1(C6orf25) c2(NP_079536) c3(protein G6b isoform G6b-A precursor) c4(218875, 218876) c5(218877, 218878, 218879, 218880, 218881, 218882, 218883, 218884, 218885, 218886, 218887, 218888, 218889, 218890, 218891, 218892, 218893, 218894, 218895, 218896, 218897, 218898, 218899, 218900, 218901, 218902, 218903, 218904, 218905) c6( ) c7(A, ac, qb, jx, eZ, fl, iV, mC, aoH); #c1(C6orf25) c2(NP_612116) c3(protein G6b isoform G6b-B precursor) c4(235878, 235879) c5(235880, 235881, 235882, 235883, 235884, 235885, 235886, 235887, 235888, 235889, 235890, 235891, 235892, 235893, 235894, 235895, 235896, 235897, 235898, 235899, 235900, 235901, 235902, 235903, 235904, 235905, 235906, 235907, 235908) c6( ) c7(A, ac, qb, jx, eZ, fL, iV, mC, aoH); #c1(C6orf25) c2(NP_612117) c3(protein G6b isoform G6b-C precursor) c4(235909, 235910) c5(235911, 235912, 235913, 235914, 235915, 235916, 235917, 235918, 235919, 235920, 235921, 235922, 235923, 235924, 235925, 235926, 235927, 235928, 235929, 235930, 235931, 235932, 235933, 235934, 235935, 235936, 235937, 235938, 235939) c6( ) c7(A, ac, qb, jx, eZ, fL, iV, mC, aoH); #c1(C6orf25) c2(NP_612118) c3(protein G6b isoform G6b-D precursor) c4(235940) c5(235941, 235942, 235943, 235944, 235945, 235946, 235947, 235948, 235949, 235950, 235951, 235952, 235953, 235954, 235955, 235956, 235957, 235958, 235959, 235960, 235961, 235962, 235963, 235964, 235965, 235966) c6( ) c7(A, ac, qb, jx, eZ, fL, iV, mC, aoH); #c1(C6orf25) c2(NP_612119) c3(protein G6b isoform G6b-E precursor) c4(235967) c5(235968, 235969, 235970, 235971, 235972, 235973, 235974, 235975, 235976, 235977, 235978, 235979, 235980, 235981, 235982, 235983, 235984, 235985, 235986, 235987, 235988, 235989, 235990, 235991, 235992, 235993) c6( ) c7(A, ac, qb, jx, eZ, fl, iV, mC, aoH); #c1(C6orf25) c2(NP_612121) c3(protein G6b isoform G6b-G precursor) c4(235994) c5(235995, 235996, 235997, 235998, 235999, 236000) c6( ) c7(A, ac, qb, jx, eZ, fL, iV, mC, aoH); #c1(C6orf47) c2(NP_067007) c3 (uncharacterized protein C6orf47) c4(208263, 208264, 208265) c5(208266, 208267, 208268, 208269, 208270, 208271, 208272, 208273, 208274, 208275, 208276, 208277, 208278, 208279, 208280, 208281, 208282, 208283, 208284, 208285, 208286, 208287, 208288, 208289, 208290, 208291, 208292, 208293, 208294, 208295, 208296, 208297, 208298, 208299, 208300, 208301) c6( ) c7(y, A, eZ); #c1(C6orf48) c2(NP_001274413) c3 (protein G8) c4(106381) c5(106382, 106383, 106384, 106385, 106386, 106387, 106388, 106389, 106390, 106391, 106392, 106393, 106394, 106395, 106396, 106397, 106398, 106399, 106400, 106401, 106402, 106403, 106404, 106405, 106406, 106407, 106408, 106409, 106410, 106411, 106412, 106413, 106414, 106415, 106416, 106417, 106418, 106419, 106420, 106421, 106422, 106423, 106424, 106425, 106426, 106427, 106428, 106429, 106430, 106431, 106432, 106433, 106434, 106435, 106436, 106437, 106438, 106439, 106440, 106441, 106442, 106443, 106444, 106445, 106446, 106447, 106448, 106449, 106450, 106451, 106452, 106453, 106454, 106455, 106456, 106457, 106458, 106459, 106460, 106461, 106462, 106463, 106464, 106465, 106466, 106467, 106468, 106469, 106470, 106471, 106472, 106473, 106474, 106475, 106476, 106477, 106478, 106479, 106480, 106481, 106482, 106483, 106484, 106485, 106486, 106487, 106488, 106489, 106490, 106491, 106492, 106493, 106494, 106495, 106496, 106497, 106498, 106499, 106500, 106501, 106502, 106503, 106504, 106505, 106506, 106507, 106508, 106509, 106510, 106511, 106512, 106513, 106514, 106515, 106516, 106517, 106518, 106519, 106520, 106521, 106522, 106523, 106524, 106525, 106526, 106527, 106528, 106529, 106530, 106531, 106532, 106533) c6( ) c7(eZ); #c1(C6orf89) c2(NP_001273564) c3(bombesin receptor-activated protein C6orf89 isoform b) c4(105294) c5(105295, 105296, 105297, 105298, 105299, 105300) c6( ) c7(cW, aw, j, ei); #c1(C6orf89) c2(NP_001273565) c3(bombesin receptor-activated protein C6orf89 isoform c) c4( ) c5(105301, 105302, 105303, 105304, 105305, 105306, 105307, 105308) c6( ) c7(cW, aw, j, ei); #c1(C6orf89) c2(NP_689947) c3(bombesin receptor-activated protein C6orf89 isoform a) c4(244450, 244451, 244452, 244453) c5(244454, 244455, 244456, 244457, 244458, 244459, 244460, 244461, 244462, 244463, 244464, 244465, 244466, 244467, 244468, 244469, 244470, 244471, 244472, 244473) c6( ) c7(cW, aw, j, ei); #c1(C7) c2(NP_000578) c3(complement component C7 precursor) c4(13132, 13133, 13134) c5(13135, 13136, 13137, 13138, 13139, 13140) c6( ) c7(W, axL, Cw, dk, aw, iW, n, pD, V, cd); #c1(C7) c2(XP_011512424) c3(complement component C7 isoform X1) c4( ) c5(303115, 303116, 303117, 303118, 303119, 303120, 303121, 303122, 303123, 303124, 303125, 303126) c6( ) c7(W, axL, Cw, dk, aw, iW, n, pD, V, cd); #c1(C7orf49) c2(NP_001230683) c3(modulator of retrovirus infection homolog isoform 3) c4(86559) c5(86560, 86561, 86562, 86563, 86564, 86565, 86566, 86567) c6( ) c7(FR); #c1(C7orf49) c2(NP_076938) c3(modulator of retrovirus infection homolog isoform 1) c4(214313, 214314) c5(214315, 214316, 214317, 214318, 214319, 214320, 214321, 214322, 214323, 214324, 214325, 214326, 214327, 214328) c6( ) c7(fR); #c1(C7orf57) c2(NP_001254795) c3(uncharacterized protein C7orf57 isoform 3) c4( ) c5(94951, 94952, 94953, 94954, 94955) c6( ) c7(c); #c1(C7orf57) c2(XP_011513423) c3(uncharacterized protein C7orf57 isoform X1) c4(305673, 305674) c5(305675, 305676, 305677, 305678,

305679, 305680, 305681, 305682, 305683, 305684, 305685, 305686) c6( ) c7(c); #c1(C7orf57) c2(XP_011513425) c3(uncharacterized protein C7orf57 isoform X4) c4(305687) c5(305688) c6( ) c7(c); #c1(C7orf60) c2(NP_689769) c3(probable methyltransferase BTM2 homolog) c4(243902, 243903) c5(243904, 243905, 243906, 243907, 243908, 243909, 243910, 243911, 243912, 243913, 243914, 243915) c6( ) c7(alv); #c1(C7orf62) c2(XP_011514206) c3(uncharacterized protein C7orf62 isoform X1) c4(306816) c5(306817, 306818, 306819, 306820, 306821, 306822) c6( ) c7(aw, ac, bA); #c1(C7orf65) c2(NP_001116537) c3(uncharacterized protein C7orf65) c4(43901) c5(43902, 43903, 43904, 43905, 43906, 43907) c6( ) c7(aw, af); #c1(C7orf69) c2(NP_079307) c3(uncharacterized protein C7orf69 isoform a precursor) c4(217730) c5(217731, 217732, 217733, 217734, 217735, 217736, 217737, 217738) c6(217739, 217740) c7(AW); #c1(C7orf72) c2(NP_001155306) c3(uncharacterized protein C7orf72) c4(61581) c5(61582, 61583, 61584, 61585, 61586, 61587) c6( ) c7(af, eZ); #c1(C8orf34) c2(NP_001182568) c3(uncharacterized protein C8orf34 isoform 2) c4(75066) c5(75067, 75068, 75069, 75070, 75071, 75072) c6( ) c7(oe, aw); #c1(C8orf34) c2(NP_443190) c3(uncharacterized protein C8orf34 isoform 1) c4(229767, 229768, 229769) c5(229770, 229771, 229772, 229773, 229774, 229775, 229776, 229777, 229778, 229779, 229780, 229781, 229782, 229783, 229784, 229785, 229786, 229787) c6( ) c7(oe, aw); #c1(C8orf34) c2(XP_011515749) c3(uncharacterized protein C8orf34 isoform X3) c4(308553) c5( ) c6( ) c7(oe, aw); #c1(C8orf34) c2(XP_011515750) c3 (uncharacterized protein C8orf34 isoform X4) c4(308554) c5(308555, 308556, 308557, 308558, 308559, 308560, 308561, 308562, 308563, 308564, 308565, 308566) c6( ) c7(oe, aw); #c1(C8orf37) c2(NP_808880) c3(protein C8orf37) c4(252988) c5(252989, 252990, 252991, 252992, 252993, 252994) c6( ) c7(aw, W, nx, cd, axM); #c1(C8orf4) c2(NP_064515) c3(uncharacterized protein C8orf4) c4(202831) c5(202832, 202833, 202834, 202835, 202836, 202837) c6( ) c7(hd, iU, nB, bE, aW, PX, aA, aT, bB, aF); #c1(C8orf46) c2(NP_689978) c3(uncharacterized protein C8orf46) c4(244638) c5(244639, 244640, 244641, 244642, 244643, 244644) c6( ) c7(dy); #c1(C8orf48) c2(NP_001007091) c3(uncharacterized protein C8orf48) c4(22484) c5(22485, 22486, 22487, 22488, 22489, 22490) c6( ) c7(oe, af, dY); #c1(C8orf86) c2(NP_997295) c3(uncharacterized protein C8orf86 isoform 1) c4(265390) c5(265391, 265392, 265393, 265394, 265395, 265396) c6( ) c7(an); #c1(C9) c2(NP_001728) c3(complement component C9 precursor) c4(120565) c5(120566, 120567, 120568, 120569, 120570, 120571) c6( ) c7(dP, ajy, Bv, VR, W, axP, axO, oV, afc, iW, jE, axN, an, B); #c1(C9orf106) c2(NP_001012733) c3(putative uncharacterized protein C9orf106) c4( ) c5(25192) c6( ) c7(aw); #c1(C9orf135) c2(NP_001010940) c3(uncharacterized protein C9orf(35) c4(24267) c5(24268, 24269, 24270, 24271, 24272, 24273) c6( ) c7(Kc); #c1(C9orf152) c2(NP_001013011) c3(uncharacterized protein C9orf152) c4(25429) c5(25430, 25431, 25432, 25433, 25434, 25435) c6( ) c7(aw); #c1(C9orf156) c2(NP_057565) c3(nef-associated protein 1) c4(191370, 191371, 191372) c5(191373, 191374, 191375, 191376, 191377, 191378, 191379) c6( ) c7(Na, cr, n, tv, Nc, tu, C, af, amL); #c1(C9orf156) c2(XP_005252107) c3(nef-associated protein 1 isoform X4) c4(271554) c5(271555, 271556, 271557, 271558, 271559) c6( ) c7(Na, cr, n, tv, Nc, tu, C, af, amL); #c1(C9orf156) c2(XP_011517078) c3(nef-associated protein 1 isoform X2) c4( ) c5(310593, 310594, 310595, 310596, 310597, 310598, 310599, 310600, 310601, 310602, 310603) c6( ) c7(Na, cr, n, tv, Nc, tu, C, af, amL); #c1(C9orf156) c2(XP_011517081) c3(nef-associated protein 1 isoform X3) c4(310604) c5(310605, 310606, 310607, 310608, 310609, 310610, 310611) c6( ) c7(Na, cr, n, tv, Nc, tu, C, af, aml); #c1(C9orf170) c2(NP_001001709) c3(uncharacterized protein C9orf170) c4(18959) c5(18960, 18961, 18962, 18963, 18964, 18965) c6( ) c7(fe, he); #c1(C9orf171) c2(NP_001269886) c3(uncharacterized protein C9orf171 isoform 2) c4(103035) c5(103036, 103037, 103038, 103039, 103040, 103041) c6( ) c7(aw, cd); #c1(C9orf171) c2(NP_997300) c3(uncharacterized protein C9orf17] isoform 1) c4(265397) c5(265398, 265399, 265400, 265401, 265402, 265403) c6( ) c7(aw, cd); #c1(C9orf3) c2(NP_001180260) c3(aminopeptidase 0 isoform 4) c4(73568, 73569) c5(73570, 73571, 73572, 73573, 73574, 73575, 73576, 73577, 73578, 73579, 73580, 73581) c6( ) c7(wL, c); #c1(C9orf3) c2(NP_116212) c3(aminopeptidase 0 isoform 2) c4(225770, 225771, 225772, 225773, 225774, 225775, 225776, 225777) c5(225778, 225779, 225780, 225781, 225782, 225783, 225784, 225785, 225786, 225787, 225788, 225789, 225790, 225791, 225792, 225793, 225794, 225795, 225796, 225797, 225798, 225799, 225800, 225801, 225802, 225803, 225804, 225805, 225806, 225807) c6( ) c7(wL, c); #c1(C9orf3) c2(XP_005252341) c3(aminopeptidase 0 isoform X5) c4( ) c5(271662, 271663, 271664, 271665, 271666, 271667) c6( ) c7(wL, c); #c1(C9orf3) c2(XP_006717369) c3(aminopeptidase 0 isoform X1) c4(291016, 291017, 291018) c5(291019, 291020, 291021, 291022, 291023, 291024, 291025, 291026, 291027, 291028, 291029, 291030) c6( ) c7(wL, c); #c1(C9orf3) c2(XP_011517425) c3 (aminopeptidase 0 isoform X7) c4(311138) c5(311139) c6( ) c7(wL, c); #c1(C9orf3) c2(XP_011517434) c3(aminopeptidase 0 isoform X16) c4( ) c5(311140, 311141, 311142, 311143, 311144, 311145, 311146, 311147, 311148, 311149, 311150) c6( ) c7(wL, c); #c1(C9orf3) c2(XP_011517437) c3(aminopeptidase 0 isoform X19) c4( ) c5(311151, 311152, 311153, 311154, 311155, 311156) c6( ) c7(wL, c); #c1(C9orf43) c2(NP_001265558) c3(uncharacterized protein C9orf43 isoform 1) c4(98835) c5(98836, 98837, 98838, 98839, 98840, 98841, 98842, 98843) c6( ) c7(Na, Nc); #c1(C9orf43) c2(NP_001265559) c3(uncharacterized protein C9orf43 isoform 2) c4( ) c5(98844, 98845) c6( ) c7(Na, Nc); #c1(C9orf66) c2(NP_689782) c3 (uncharacterized protein C9orf66) c4(243939) c5(243940, 243941, 243942, 243943, 243944, 243945) c6( ) c7(cd); #c1(C9orf72) c2(NP_060795) c3(protein C9orf72 isoform a) c4(197873, 197874) c5(197875, 197876, 197877, 197878, 197879, 197880) c6( ) c7(iH, Tb, Ol, oe, yY, GC, n, Vd, j, nA, PJ, V, PW, aV, WY, sO, axU, bK, K, an, he, af, axR, axW, tx, TO, gl, aaE, axl, axT, xw, avz, axS, bb, c, aaG, U, axV, act, y, gD); #c1(C9orf72) c2(NP_659442) c3(protein C9orf72 isoform b) c4( ) c5(239915, 239916, 239917, 239918, 239919, 239920, 239921, 239922, 239923, 239924) c6( ) c7(iH, Tb, Ol, oe, yY, GC, n, Vd, j, nA, PJ, V, PW, aV, WY, s, axU, bK, K, an, he, af, axR, axW, tx, TO, gl, aaE, axl, axT, xw, avz, axS, bb, c, aaG, U, axV, act, y, gD); #c1(C9orf85) c2(NP_872311) c3 (uncharacterized protein C9orf85) c4(256087, 256088) c5(256089, 256090, 256091, 256092, 256093, 256094, 256095, 256096, 256097, 256098, 256099, 256100) c6( ) c7(De); #c1(C9orf85) c2(XP_011516529) c3(uncharacterized protein C9orf85 isoform X2) c4(309946) c5( ) c6( ) c7(De); #c1(C9orf85) c2(XP_011516530) c3(uncharacterized protein C9orf85 isoform X3) c4( ) c5(309947, 309948, 309949, 309950, 309951, 309952) c6( ) c7(oe); #c1(C9orf9) c2(NP_061829) c3(uncharacterized protein C9orf9) c4(200638) c5(200639, 200640, 200641, 200642, 200643, 200644) c6( ) c7(Xd, ok, ao); #c1(C9orf91) c2(NP_694590) c3(transmembrane protein C9orf91) c4(245337) c5(245338, 245339, 245340, 245341, 245342, 245343) c6( ) c7(AW); #c1(C9orf91) c2(XP_006717060) c3(transmembrane protein C9orf91 isoform X4) c4(290664) c5(290665, 290666, 290667, 290668, 290669, 290670) c6( ) c7(AW); #c1(CAI) c2(XP_011515886) c3(carbonic anhydrase 1 isoform X1) c4(308904, 308905, 308906, 308907, 308908, 308909, 308910, 308911) c5(308912, 308913, 308914, 308915, 308916, 308917, 308918, 308919, 308920, 308921, 308922, 308923, 308924, 308925, 308926, 308927, 308928, 308929, 308930, 308931, 308932, 308933, 308934, 308935, 308936, 308937, 308938, 308939, 308940, 308941, 308942, 308943, 308944, 308945, 308946, 308947, 308948, 308949, 308950, 308951, 308952, 308953, 308954, 308955, 308956, 308957, 308958, 308959, 308960, 308961, 308962, 308963, 308964, 308965, 308966, 308967, 308968, 308969, 308970, 308971, 308972, 308973, 308974, 308975, 308976, 308977, 308978, 308979, 308980, 308981, 308982, 308983) c6( ) c7(bE, NT, hr, aA, mE, r, bB, vy, dy, ci, g, iV, y, it, q); #c1(CA10) c2(NP_001076003) c3(carbonic anhydrase-related protein 10 precursor) c4(37288, 37289) c5(37290, 37291, 37292, 37293, 37294, 37295, 37296, 37297, 37298, 37299, 37300, 37301, 37302, 37303, 37304, 37305, 37306, 37307, 37308, 37309) c6( ) c7(qw, ac, aW, aw, aT, v, Gp, fb, fq, bA); #c1(CA11) c2(NP_001208) c3(carbonic anhydrase-related protein II precursor) c4(83732) c5(83733, 83734, 83735, 83736, 83737, 83738, 83739, 83740, 83741) c6( ) c7(T, Kv, aW, if, aA, aT, aF, qN, cm, fb); #c1(CA12) c2(NP_001209) c3(carbonic anhydrase 12 isoform 1 precursor) c4(83742) c5(83743, 83744, 83745, 83746, 83747, 83748) c6( ) c7(T, ac, nB, ig, aW, if, aT, axX, n, axY, cd, fr, qN, as, dw, AO, aed, B); #c1(CA12) c2(NP_001280571) c3(carbonic anhydrase (2 isoform 3 precursor) c4(111536) c5(111537, 111538, 111539, 111540, 111541, 111542) c6( ) c7(T, ac, nB, ig, aW, if, aT, axX, n, axY, cd, fr, qN, as, dw, AO, aed, B); #c1(CA12) c2(NP_996808) c3(carbonic anhydrase 12 isoform 2 precursor) c4(264605) c5(264606, 264607, 264608, 264609, 264610, 264611) c6( ) c7(T, ac, nB, ig, aW, if, aT, axX, n, axY, cd, fr, qN, as, dw, AO, aed, B); #c1(CA13) c2(NP_940986) c3(carbonic anhydrase 13) c4(260260) c5(260261, 260262, 260263, 260264, 260265, 260266) c6( ) c7(W, aW, r, aT, fd, y, q); #c1(CA2) c2(NP_000058) c3(carbonic anhydrase 2 isoform 1) c4(1073) c5(1074, 1075, 1076, 1077, 1078, 1079) c6( ) c7(T, aC, id, DD, zs, e, Kk, aoE, aA, cR, j, nA, eJ, qH, dP, aE, hb, dL, K, eL, af, dx, bA, ao, jf, xd, iF, A, s, ajT, cW, cr, ge, axZ, g, cl, eE, f, iV, dr, OC, ar, mO, pg, cS, dy, pw, be, jM, y, it, B, jr, aya); #c1(CA3) c2(NP_005172) c3(carbonic anhydrase 3) c4(153622, 153623) c5(153624, 153625, 153626, 153627, 153628, 153629, 153630, 153631, 153632, 153633, 153634, 153635) c6( ) c7(jp, aw, fQ, uY, aq, v, iR, B); #c1(CA4) c2(NP_000708) c3(carbonic anhydrase 4 precursor) c4(14745) c5(14746, 14747, 14748, 14749, 14750, 14751) c6( ) c7(xX, ayb, lR, DD, wV, fr, K, m, t, nC, X, de); #c1(CASA) c2(NP_001730) c3(carbonic anhydrase 5A, mitochondrial precursor) c4(120572) c5(120573, 120574, 120575, 120576, 120577, 120578) c6( ) c7(T, oW, bK, dw, ayc, qW, is); #c1(CA6) c2(XP_011540385) c3(carbonic anhydrase 6 isoform X1) c4( ) c5(338880, 338881, 338882, 338883, 338884, 338885, 338886, 338887) c6( ) c7(ka, ki, rl, r, aye, xY, ayd); #c1(CAB) c2(NP_004047) c3 (carbonic anhydrase-related protein) c4(143143) c5(143144, 143145, 143146, 143147, 143148, 143149) c6( ) c7(bv, T, cc, ky, s, bE, eM, Y, X, mx, adl, r, bB, im, nA, g, q, MG, bA, ayf); #c1(CAB39) c2(NP_001124322) c3(calcium-binding protein 39) c4(48667) c5(48668, 48669, 48670, 48671, 48672, 48673, 48674, 48675, 48676, 48677, 48678, 48679, 48680, 48681) c6( ) c7(T, ey, eA, MC); #c1(CAB39L) c2(NP_001274268) c3(calcium-binding protein 39-like) c4(106149, 106150, 106151, 106152, 106153) c5(106154, 106155, 106156, 106157, 106158, 106159, 106160, 106161, 106162, 106163, 106164, 106165, 106166, 106167, 106168, 106169, 106170, 106171, 106172, 106173, 106174, 106175, 106176, 106177, 106178, 106179, 106180, 106181, 106182, 106183, 106184, 106185, 106186, 106187, 106188, 106189, 106190, 106191, 106192, 106193, 106194, 106195) c6( ) c7(aC, iZ); #c1(CABIN1) c2(NP_036427) c3(calcineurin-binding protein cabin-1 isoform a) c4(173784) c5(173785, 173786, 173787, 173788, 173789, 173790, 173791, 173792, 173793, 173794) c6( ) c7(fU, ai, eR, c, cH, aW, eE, jx, aT, cz, n, cd, m, Hb, ci, t, fy, EA, ayg, B, vi); #c1(CABIN1) c2(XP_005261477) c3(calcineurin-binding protein cabin-1 isoform X6) c4(277368) c5(277369, 277370, 277371, 277372, 277373, 277374) c6( ) c7(fU, ai, eR, c, cH, aW, eE, jx, aT, cz, n, cd, m, Hb, ci, t, fy, EA, ayg, B, vi); #c1 (CABIN1) c2(XP_006724245) c3(calcineurin-binding protein cabin-1 isoform X8) c4(295874) c5( ) c6( ) c7(fU, ai, eR, c, cH, aW, eE, jx, aT, cz, n, cd, m, Hb, ci, t, fy, EA, ayg, B, vi); #c1(CABIN1) c2(XP_006724247) c3(calcineurin-binding protein cabin-1 isoform X18) c4( ) c5(295875, 295876, 295877, 295878, 295879, 295880) c6( ) c7(fU, ai, eR, c, cH, aW, eE, jx, aT, cz, n, cd, m, Hb, ci, t, fy, EA, ayg, B, vi); #c1(CABLES1) c2(NP_001094089) c3(CDK5 and ABL1 enzyme substrate 1 isoform 2) c4(40634, 40635) c5(40636, 40637, 40638, 40639, 40640, 40641) c6( ) c7(T, ac, bE, X, r, bB, m, cl, t, Yr, q, de); #c1(CABLES1) c2(NP_001243367) c3(CDK5 and ABL1 enzyme substrate 1 isoform 3) c4( ) c5(90580, 90581, 90582, 90583, 90584) c6( ) c7(T, ac, bE, X, r, bB, m, cl, t, Yr, q, le); #c1(CABLES1) c2(NP_612384) c3(CDK5 and ABL1 enzyme substrate 1 isoform 1) c4(236320) c5(236321, 236322, 236323, 236324, 236325, 236326, 236327) c6( ) c7(T, ac, bE, X, r, bB, m, cl, t, Yr, q, de); #c1(CABP2) c2(NP_057450) c3(calcium-binding protein 2) c4(190946) c5(190947, 190948, 190949, 190950, 190951, 190952) c6( ) c7(cm, ayh); #c1(CABP4) c2(NP_001287824) c3(calcium-binding protein 4 isoform b) c4(112924) c5(112925, 112926, 112927, 112928, 112929, 112930) c6( ) c7(cm, ayj, ayi, lR); #c1(CABP4) c2(NP_660201) c3(calcium-binding protein 4 isoform a) c4(240503) c5(240504, 240505, 240506, 240507, 240508, 240509) c6( ) c7(cm, ayj, ayi, lR); #c1(CABS1) c2(NP_149113) c3(calcium-binding and spermatid-specific protein 1) c4(227218) c5(227219, 227220, 227221, 227222, 227223, 227224) c6( ) c7(aw); #c1(CABYR) c2(NP_036321) c3(calcium-binding tyrosine phosphorylation-regulated protein isoform a) c4(172981, 172982) c5(172983, 172984, 172985, 172986, 172987, 172988) c6( ) c7(dB, T, bz, bu); #c1(CABYR) c2(NP_619584) c3(calcium-binding tyrosine phosphorylation-regulated protein isoform d) c4( ) c5(236894, 236895, 236896, 236897, 236898) c6( ) c7(dB, T, bz, bu); #c1(CABYR) c2(NP_722452) c3(calcium-binding tyrosine phosphorylation-regulated protein isoform b) c4(246731) c5(246732, 246733, 246734, 246735, 246736, 246737, 246738) c6( ) c7(dB, T, bz, bu); #c1(CABYR) c2(NP_722453) c3(calcium-binding tyrosine phosphorylation-regulated protein isoform c) c4(246739, 246740) c5(246741, 246742, 246743, 246744, 246745, 246746, 246747, 246748, 246749, 246750) c6( ) c7(dB, T, bz, bu); #c1(CABYR) c2(NP_722454) c3(calcium-binding tyrosine phosphorylation-regulated protein isoform e) c4(246751) c5(246752, 246753, 246754, 246755, 246756, 246757) c6( ) c7(dB, T, bz, bu); #c1(CABYR) c2(XP_005258304) c3(calcium-binding tyrosine phosphorylation-regulated protein isoform X3) c4( ) c5(275626) c6( ) c7(dB, T, bz, bu); #c1(CACNA1A) c2(NP_001120693) c3(voltage-dependent P/Q-type calcium channel subunit alpha-1A isoform 3) c4(44603) c5(44604, 44605, 44606, 44607, 44608, 44609, 44610, 44611, 44612, 44613, 44614, 44615) c6( ) c7(dt, yY, ha, cV, rf, lb, n, acU, OY, Kh, ur, akg, nA, uu, auu, yU, aR, aym, ad, av, no, aV, ayk, aU, ayp, yZ, dL, bK, hg, an, akr, ae, af, eL, aIL, Ng, yr, ayl, XK, Ub, ayn, akL, EH, la, akh, RI, aqP, vo, nC, ayj, dr, akp, Cq, KE, ayq, ky, aki, kB, rR, Kv, akm, HN, U, nw, mk, ayo); #c1(CACNA1A) c2(NP_001120694) c3(voltage-dependent P/Q-type calcium channel subunit alpha-1A isoform 4) c4(44616, 44617, 44618) c5(44619, 44620, 44621, 44622, 44623, 44624, 44625, 44626, 44627, 44628, 44629, 44630) c6( ) c7(dt, yY, ha, cV, rf, lb, n, acu, OY, Kh, ur, akg, nA, uu, auu, yU, aR, aym, ad, av, no, aV, ayk, aU, ayp, yZ, dL, bK, hg, an, akr, ae, af, eL, aIL, Ng, yr, ayl, XK, Ub, ayn, akL, EH, la, akh, RI, aqP, vo, nC, ayj, dr, akp, Cq, KE, ayq, ky, aki, kB, rR, Kv, akm, HN, U, nw, mk, ayo); #c1(CACNA1A) c2(NP_075461) c3(voltage-dependent P/Q-type calcium channel subunit alpha-1A isoform 2) c4(213881) c5(213882, 213883, 213884, 213885, 213886, 213887) c6( ) c7(dt, yY, ha, cV, rf, lb, n, acI, OY, Kh, ur, akg, nA, uu, auu, yU, aR, aym, ad, av, no, aV, ayk, aU, ayp, yZ, dL, bK, hg, an, akr, ae, af, eL, aIL, Ng, yr, ayl, XK, Ub, ayn, akL, EH, la, akh, RI, aqP, vo, nC, ayj, dr, akp, Cq, KE, ayq, ky, aki, kB, rR, Kv, akm, HN, U, nw, mk, ayo); #c1(CACNA1B) c2(NP_000709) c3(voltage-dependent N-type calcium channel subunit alpha-18 isoform 1) c4(14752) c5(14753, 14754, 14755, 14756, 14757, 14758) c6( ) c7(Fg, s, bX); #c1(CACNA1B) c2(NP_001230741) c3(voltage-dependent N-type calcium channel subunit alpha-1B isoform 2) c4(86745) c5(86746, 86747, 86748, 86749, 86750, 86751) c6( ) c7(Fg, s, bX); #c1(CACNA1B) c2(XP_011517292) c3(voltage-dependent N-type calcium channel subunit alpha-18 isoform X1) c4( ) c5(310893, 310894, 310895, 310896, 310897, 310898, 310899) c6( ) c7(Fg, s, bX); #c1(CACNA1B) c2(XP_011517293) c3(voltage-dependent N-type calcium channel subunit alpha-1B isoform X2) c4(310900) c5(310901, 310902, 310903, 310904, 310905, 310906) c6( ) c7(Fg, s, bX); #c1(CACNA1B) c2(XP_011517295) c3(voltage-dependent N-type calcium channel subunit alpha-18 isoform X4) c4(310907, 310908) c5(310909, 310910, 310911, 310912, 310913, 310914) c6( ) c7(Fg, s, bX); #c1(CACNA1B) c2(XP_011517296) c3(voltage-dependent N-type calcium channel subunit alpha-18 isoform X5) c4(310915) c5(310916, 310917, 310918, 310919, 310920, 310921, 310922, 310923, 310924, 310925, 310926, 310927) c6( ) c7(Fg, s, bX); #c1(CACNA1B) c2(XP_011517297) c3(voltage-dependent N-type calcium channel subunit alpha-1B isoform X6) c4(310928, 310929) c5(310930, 310931, 310932, 310933, 310934, 310935, 310936, 310937, 310938, 310939, 310940, 310941) c6( ) c7(Fg, s, bX); #c1(CACNA1B) c2(XP_011517298) c3(voltage-dependent N-type calcium channel subunit alpha-1B isoform X7) c4( ) c5(310942, 310943, 310944, 310945, 310946, 310947, 310948, 310949, 310950, 310951, 310952, 310953, 310954) c6( ) c7(Fg, s, bX); #c1(CACNA1B) c2(XP_011517299) c3(voltage-dependent N-type calcium channel subunit alpha-1B isoform X8) c4(310955, 310956, 310957) c5( ) c6( ) c7(Fg, s, bX); #c1(CACNA1C) c2(NP_000710) c3(voltage-dependent L-type calcium channel subunit alpha-1C isoform 18) c4(14759) c5(14760, 14761, 14762, 14763, 14764, 14765, 14766) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1C) c2(NP_001123299) c3(voltage-dependent L-type calcium channel subunit alpha-IC isoform 2) c4(46869) c5(46870, 46871, 46872, 46873, 46874, 46875, 46876) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, OV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1C) c2(NP_001123301) c3(voltage-dependent L-type calcium channel subunit alpha-1C isoform 3) c4(46884) c5(46885, 46886, 46887, 46888, 46889, 46890, 46891) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1C) c2(NP_001123302) c3(voltage-dependent L-type calcium channel subunit alpha-1C isoform 4) c4(46892) c5(46893, 46894, 46895, 46896, 46897, 46898, 46899) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1C) c2(NP_001123303) c3(voltage-dependent L-type calcium channel subunit alpha-1C isoform 5) c4(46900) c5(46901, 46902, 46903, 46904, 46905, 46906, 46907) c6( ) c7(bL, T, dD, fR, ms, aw, ays, cV, cz, arD, bO, ajM, bS, gf, ct, nA, zu, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1C) c2(NP_001123304) c3(voltage-dependent L-type calcium channel subunit alpha-1C isoform 6) c4(46908) c5(46909, 46910, 46911, 46912, 46913, 46914, 46915) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1C) c2(NP_001123305) c3(voltage-dependent L-type calcium channel subunit alpha-1C isoform 7) c4(46916) c5(46917, 46918, 46919, 46920, 46921, 46922, 46923) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1C) c2(NP_001123306) c3(voltage-dependent L-type calcium channel subunit alpha-1C isoform 8) c4(46924) c5(46925, 46926, 46927, 46928, 46929, 46930, 46931) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, OV, vj, lW, Az, fe, ayr, gl, Z, vo, dr, atb, HT, hq, qa, bN, TA, ayt, ac, cA, bo, Kr, wB, vL, gZ, uJ, xg); #c1 (CACNA1C) c2(NP_001123307) c3(voltage-dependent L-type calcium channel subunit alpha-1C isoform 9) c4(46932, 46933) c5(46934, 46935, 46936, 46937, 46938, 46939, 46940) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1C) c2(NP_001123308) c3(voltage-dependent L-type calcium channel subunit alpha-1C isoform 10) c4(46941) c5(46942, 46943, 46944, 46945, 46946, 46947, 46948) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1C) c2(NP_001123309) c3(voltage-dependent L-type calcium channel subunit alpha-1C isoform 11) c4(46949) c5(46950, 46951, 46952, 46953, 46954, 46955, 46956) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1C) c2(NP_001123310) c3(voltage-dependent L-type calcium channel subunit alpha-1C isoform 12) c4(46957) c5(46958, 46959, 46960, 46961, 46962, 46963, 46964) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1 (CACNA1C) c2(NP_001123311) c3(voltage-dependent L-type calcium channel subunit alpha-1C isoform 13) c4(46965) c5(46966, 46967, 46968, 46969, 46970, 46971, 46972) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1C) c2(NP_001123312) c3(voltage-dependent L-type calcium channel subunit alpha-1C isoform 14) c4(46973) c5(46974, 46975, 46976, 46977, 46978, 46979, 46980) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, OV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1C) c2(NP_001123313) c3(voltage-dependent L-type calcium channel subunit alpha-1C isoform 15) c4(46981) c5(46982, 46983, 46984, 46985, 46986, 46987, 46988, 46989, 46990, 46991, 46992, 46993, 46994) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1 (CACNA1C) c2(NP_001123314) c3(voltage-dependent L-type calcium channel subunit alpha-1C isoform 16) c4(46995) c5(46996, 46997, 46998, 46999, 47000, 47001, 47002) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1C) c2(NP_001123315) c3(voltage-dependent L-type calcium channel subunit alpha-1C isoform (7) c4(47003) c5(47004, 47005, 47006, 47007, 47008, 47009, 47010) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1C) c2(NP_001123316) c3(voltage-dependent L-type calcium channel subunit alpha-IC isoform 19) c4( ) c5(47011) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1C) c2(NP_001123318) c3(voltage-dependent L-type calcium channel subunit alpha-1C isoform 20) c4( ) c5(47012) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1C) c2(NP_001161095) c3(voltage-dependent L-type calcium channel subunit alpha-1C isoform 21) c4(65321) c5(65322, 65323, 65324, 65325, 65326, 65327, 65328) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1C) c2(NP_001161096) c3(voltage-dependent L-type calcium channel subunit alpha-1C isoform 22) c4(65329) c5(65330, 65331, 65332, 65333, 65334, 65335, 65336) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1C) c2(NP_001161097) c3(voltage-dependent L-type calcium channel subunit alpha-1C isoform 23) c4(65337, 65338) c5(65339, 65340, 65341, 65342, 65343, 65344, 65345, 65346, 65347, 65348, 65349, 65350, 65351) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1 (CACNA1C) c2(NP_955630) c3(voltage-dependent L-type calcium channel subunit alpha-1C isoform 1) c4( ) c5(261910) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, OV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1C) c2(XP_011519324) c3(voltage-dependent L-type calcium channel subunit alpha-1C isoform X7) c4(313973) c5(313974, 313975, 313976, 313977, 313978, 313979, 313980) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1C) c2(XP_011519325) c3(voltage-dependent L-type calcium channel subunit alpha-1C isoform X8) c4(313981) c5(313982, 313983, 313984, 313985, 313986, 313987) c6( ) c7(bL, T, dD, fR, ms, aw, ays, cV, cz, arD, bO, ajM, bS, gf, ct, nA, zU, uu, eq, aR, ke, cF, pN, Fo, vc, bK, K, he, Fg, rz, VP, mu, Cw, oV, vj, lW, Az, fe, ayr, gl, Z, vo, dr, atb, HT, hq, qa, bN, TA, ayt, ac, cA, bo, Kr, wB, vL, gZ, uJ, xg); #c1(CACNA1D) c2(NP_000711) c3(voltage-dependent L-type calcium channel subunit alpha-1D isoform a) c4(14767) c5(14768, 14769, 14770, 14771, 14772, 14773) c6( ) c7(bL, mj, ot, s, Fj, vj, My, Ak, ayu, cz, cv, dG, ayv, Fg, qa, mu); #c1(CACNA1D) c2(NP_001122311) c3(voltage-dependent L-type calcium channel subunit alpha-1D isoform c) c4(46489, 46490) c5(46491, 46492, 46493, 46494, 46495, 46496, 46497, 46498, 46499, 46500, 46501, 46502) c6( ) c7(bL, mj, ot, s, Fj, vj, My, Ak, ayu, cz, cv, dG, ayv, Fg, qa, mu); #c1(CACNA1D) c2(NP_001122312) c3(voltage-dependent L-type calcium channel subunit alpha-1D isoform b) c4(46503) c5(46504, 46505, 46506, 46507, 46508, 46509) c6( ) c7(bL, mj, ot, s, Fj, vj, My, Ak, ayu, cz, cv, dG, ayv, Fg, qa, mu); #c1(CACNA1D) c2(XP_005265505) c3(voltage-dependent L-type calcium channel subunit alpha-1D isoform X4) c4(279568) c5( ) c6( ) c7(bL, mj, at, s, Fj, vj, My, Ak, ayu, cz, cv, dG, ayv, Fg, qa, mu); #c1(CACNA1D) c2(XP_011532396) c3(voltage-dependent L-type calcium channel subunit alpha-1D isoform X1) c4(328581) c5(328582, 328583, 328584, 328585, 328586, 328587, 328588) c6( ) c7(bL, mj, ot, s, Fj, vj, My, Ak, ayu, cz, cv, dG, ayv, Fg, qa, mu); #c1(CACNA1D) c2(XP_011532401) c3(voltage-dependent L-type calcium channel subunit alpha-1D isoform X6) c4( ) c5(328589, 328590, 328591, 328592, 328593, 328594) c6( ) c7(bL, mj, ot, s, Fj, vj, My, Ak, ayu, cz, cv, dG, ayv, Fg, qa, mu); #c1(CACNA1E) c2(NP_000712) c3(voltage-dependent R-type calcium channel subunit alpha-1E isoform 3) c4(14774, 14775) c5(14776, 14777, 14778, 14779, 14780, 14781, 14782, 14783, 14784, 14785, 14786, 14787) c6( ) c7(bL, vj, jN, mf, ur, vo); #c1(CACNA1E) c2(NP_001192222) c3(voltage-dependent R-type calcium channel subunit alpha-1E isoform 1) c4(82084, 82085, 82086) c5(82087, 82088, 82089, 82090, 82091, 82092, 82093, 82094, 82095, 82096, 82097, 82098, 82099) c6( ) c7(bL, vj, jN, mf, ur, vo); #c1(CACNA1E) c2(NP_001192223) c3(voltage-dependent R-type calcium channel subunit alpha-1E isoform 2) c4(82100, 82101) c5(82102, 82103, 82104, 82105, 82106, 82107, 82108, 82109, 82110, 82111, 82112, 82113, 82114, 82115, 82116, 82117, 82118, 82119, 82120) c6( ) c7(bL, vj, jN, mf, ur, vo); #c1(CACNA1E) c2(XP_011508273) c3(voltage-dependent R-type calcium channel subunit alpha-1E isoform X1) c4(298965) c5(298966, 298967, 298968, 298969, 298970, 298971) c6( ) c7(bL, vj, jN, mf, ur, vo); #c1(CACNA1F) c2(NP_001243718) c3(voltage-dependent L-type calcium channel subunit alpha-1F isoform 2) c4(91533) c5(91534, 91535, 91536, 91537, 91538, 91539) c6( ) c7(ayx, cZ, ayw, cV, HU, ayA, dd, d, lR, ayB, lW, qL, ayr, as, ayy, nC, ayj, KE, Be, ayz, awK, oQ); #c1(CACNA1F) c2(NP_001243719) c3(voltage-dependent L-type calcium channel subunit alpha-1F isoform 3) c4(91540) c5(91541, 91542, 91543, 91544, 91545, 91546) c6( ) c7(ayx, cZ, ayw, cV, HU, ayA, dd, d, lR, ayB, lW, qL, ayr, as, ayy, nC, ayj, KE, Be, ayz, awk, oQ); #c1(CACNA1F) c2(NP_005174) c3(voltage-dependent L-type calcium channel subunit alpha-1F isoform 1) c4(153636) c5(153637, 153638, 153639, 153640, 153641, 153642) c6( ) c7(ayx, cZ, ayw, cV, HU, ayA, dd, d, lR, ayB, lW, qL, ayr, as, ayy, nC, ayj, KE, Be, ayz, awk, oQ); #c1(CACNA1G) c2(NP_001243253) c3(voltage-dependent T-type calcium channel subunit alpha-1G isoform 16) c4(90252) c5(90253, 90254, 90255, 90256, 90257, 90258, 90259, 90260, 90261, 90262, 90263, 90264) c6( ) c7(bL, T, Cq, A, s, ha, HN, EQ, r, aA, n, bB, hg, nA, g, bN); #c1(CACNA1G) c2(NP_001243254) c3(voltage-dependent T-type calcium channel subunit alpha-1G isoform (7) c4(90265) c5(90266, 90267, 90268, 90269, 90270, 90271) c6( ) c7(bL, T, Cq, A, s, ha, HN, EQ, r, aA, n, bB, hg, nA, g, bN); #c1(CACNA1G) c2(NP_001243255) c3(voltage-dependent T-type calcium channel subunit alpha-1G isoform 18) c4(90272) c5(90273, 90274, 90275, 90276, 90277, 90278) c6( ) c7(bL, T, Cq, A, s, ha, HN, EQ, r, aA, n, bB, hg, nA, g, bN); #c1(CACNA1G) c2(NP_001243256) c3(voltage-dependent T-type calcium channel subunit alpha-1G isoform 19) c4(90279) c5(90280, 90281, 90282, 90283, 90284, 90285) c6( ) c7(bL, T, Cq, A, s, ha, HN, EQ, r, aA, n, bB, hg, nA, g, bN); #c1(CACNA1G) c2(NP_001243257) c3(voltage-dependent T-type calcium channel subunit alpha-1G isoform 20) c4(90286) c5(90287, 90288, 90289, 90290, 90291, 90292) c6( ) c7(bL, T, Cq, A, s, ha, HN, EQ, r, aA, n, bB, hg, nA, g, bN); #c1(CACNA1G) c2(NP_001243258) c3(voltage-dependent T-type calcium channel subunit alpha-16 isoform 21) c4(90293) c5(90294, 90295, 90296, 90297, 90298, 90299) c6( ) c7(bL, T, Cq, A, s, ha, HN, EQ, r, aA, n, bB, hg, nA, g, bN); #c1(CACNA1G) c2(NP_001243259) c3(voltage-dependent T-type calcium channel subunit alpha-1G isoform 22) c4(90300) c5(90301, 90302, 90303, 90304, 90305, 90306) c6( ) c7(bL, T, Cq, A, s, ha, HN, EQ, r, aA, n, bB, hg, nA, g, bN); #c1(CACNA1G) c2(NP_001243260) c3(voltage-dependent T-type calcium channel subunit alpha-1G isoform 23) c4(90307) c5(90308, 90309, 90310, 90311, 90312, 90313) c6( ) c7(bL, T, Cq, A, s, ha, HN, EQ, r, aA, n, bB, hg, nA, g, bN); #c1(CACNA1G) c2(NP_001243261) c3(voltage-dependent T-type calcium channel subunit alpha-1G isoform 24) c4(90314) c5(90315, 90316, 90317, 90318, 90319, 90320) c6( ) c7(bL, T, Cq, A, s, ha, HN, EQ, r, aA, n, bB, hg, nA, g, bN); #c1(CACNA1G) c2(NP_001243262) c3(voltage-dependent T-type calcium channel subunit alpha-1G isoform 25) c4(90321) c5(90322, 90323, 90324, 90325, 90326, 90327) c6( ) c7(bL, T, Cq, A, s, ha, HN, EI, r, aA, n, bB, hg, nA, g, bN); #c1(CACNA1G) c2(NP_001243263) c3(voltage-dependent T-type calcium channel subunit alpha-1G isoform 26) c4(90328, 90329) c5(90330, 90331, 90332, 90333, 90334, 90335) c6( ) c7(bL, T, Cq, A, s, ha, HN, EQ, r, aA, n, bB, hg, nA, g, bN); #c1(CACNA1G) c2(NP_061496) c3(voltage-dependent T-type calcium channel subunit alpha-1G isoform 1) c4(200363, 200364) c5(200365, 200366, 200367, 200368, 200369, 200370, 200371, 200372, 200373, 200374, 200375, 200376) c6( ) c7(bL, T, Cq, A, s, ha, HN, EQ, r, aA, n, bB, hg, nA, g, bN); #c1(CACNA1G) c2(NP_938190) c3(voltage-dependent T-type calcium channel subunit alpha-1G isoform 14) c4(259660) c5(259661, 259662, 259663, 259664, 259665, 259666) c6( ) c7(bL, T, Cq, A, s, ha, HN, EQ, r, aA, n, bB, hg, nA, g, bN); #c1(CACNA1G) c2(NP_938191) c3(voltage-dependent T-type calcium channel subunit alpha-1G isoform 2) c4(259667, 259668, 259669) c5(259670, 259671, 259672, 259673, 259674, 259675) c6( ) c7(bL, T, Cq, A, s, ha, HN, EQ, r, aA, n, bB, hg, nA, g, bN); #c1(CACNA1G) c2(NP_938192) c3(voltage-dependent T-type calcium channel subunit alpha-1G isoform 9) c4(259676) c5(259677, 259678, 259679, 259680, 259681, 259682, 259683, 259684, 259685, 259686, 259687, 259688) c6( ) c7(bL, T, Cq, A, s, ha, HN, EQ, r, aA, n, bB, hg, nA, g, bN); #c1 (CACNA1G) c2(NP_938193) c3(voltage-dependent T-type calcium channel subunit alpha-1G isoform 7) c4(259689) c5(259690, 259691, 259692, 259693, 259694, 259695) c6( ) c7(bL, T, Cq, A, s, ha, HN, EQ, r, aA, n, bB, hg, nA, g, bN); #c1(CACNA1G) c2(NP_938194) c3(voltage-dependent T-type calcium channel subunit alpha-1G isoform 5) c4(259696) c5(259697, 259698, 259699, 259700, 259701, 259702) c6( ) c7(bL, T, Cq, A, s, ha, HN, EQ, r, aA, n, bB, hg, nA, g, bN); #c1(CACNA1G) c2(NP_938196) c3(voltage-dependent T-type calcium channel subunit alpha-1G isoform 11) c4(259713) c5(259714, 259715, 259716, 259717, 259718, 259719, 259720, 259721, 259722, 259723, 259724, 259725, 259726, 259727, 259728, 259729, 259730, 259731) c6( ) c7(bL, T, Cq, A, s, ha, HN, EQ, r, aA, n, bB, hg, nA, g, bN); #c1(CACNA1G) c2(NP_938197) c3(voltage-dependent T-type calcium channel subunit alpha-1G isoform 6) c4(259732, 259733, 259734) c5(259735, 259736, 259737, 259738, 259739, 259740) c6( ) c7(bL, T, Cq, A, s, ha, HN, EQ, r, aA, n, bB, hg, nA, g, bN); #c1 (CACNA1G) c2(NP_938198) c3(voltage-dependent T-type calcium channel subunit alpha-1G isoform 8) c4(259741) c5(259742, 259743, 259744, 259745, 259746, 259747) c6( ) c7(bL, T, Cq, A, s, ha, HN, EQ, r, aA, n, bB, hg, nA, g, bN); #c1(CACNA1G) c2(NP_938199) c3(voltage-dependent T-type calcium channel subunit alpha-1G isoform 4) c4(259748) c5(259749, 259750, 259751, 259752, 259753, 259754) c6( ) c7(bL, T, Cq, A, s, ha, HN, EQ, r, aA, n, bB, hg, nA, g, bN); #c1(CACNA1G) c2(NP_938200) c3(voltage-dependent T-type calcium channel subunit alpha-1G isoform 10) c4(259755) c5(259756, 259757, 259758, 259759, 259760, 259761) c6( ) c7(bL, T, Cq, A, s, ha, HN, EQ, r, aA, n, bB, hg, nA, g, bN); #c1(CACNA1G) c2(NP_938201) c3(voltage-dependent T-type calcium channel subunit alpha-1G isoform 12) c4(259762, 259763) c5(259764, 259765, 259766, 259767, 259768, 259769) c6( ) c7(bL, T, Cq, A, s, ha, HN, EQ, r, aA, n, bB, hg, nA, g, bN); #c1(CACNA1G) c2(NP_938202) c3(voltage-dependent T-type calcium channel subunit alpha-1G isoform 13) c4(259770) c5(259771, 259772, 259773, 259774, 259775, 259776, 259777, 259778, 259779, 259780, 259781, 259782) c6( ) c7(bL, T, Cq, A, s, ha, HN, EQ, r, aA, n, bB, hg, nA, g, bN); #c1(CACNA1G) c2(NP_938406) c3(voltage-dependent T-type calcium channel subunit alpha-1G isoform 3) c4(259815) c5(259816, 259817, 259818, 259819, 259820, 259821) c6( ) c7(bL, T, Cq, A, s, ha, HN, EQ, r, aA, n, bB, hg, nA, g, bN); #c1(CACNA1G) c2(XP_011523742) c3(voltage-dependent T-type calcium channel subunit alpha-1G isoform X1) c4( ) c5(318238, 318239, 318240, 318241, 318242, 318243) c6( ) c7(bL, T, Cq, A, s, ha, HN, EQ, r, aA, n, bB, hg, nA, g, bN); #c1(CACNA1H) c2(NP_001005407) c3(voltage-dependent T-type calcium channel subunit alpha-1H isoform b) c4(21647) c5(21648, 21649, 21650, 21651, 21652, 21653) c6( ) c7(ae, Cq, dD, s, xZ, ha, HN, aT, cz, ayD, eq, gu, hg, rz, Fg, ayC, ad, bN); #c1 (CACNA1H) c2(NP_066921) c3(voltage-dependent T-type calcium channel subunit alpha-1H isoform a) c4(207478) c5(207479, 207480, 207481, 207482, 207483, 207484) c6( ) c7(ae, Cq, dD, s, xZ, ha, HN, aT, cz, ayD, eq, gu, hg, rz, Fg, ayC, ad, bN); #c1(CACNA1H) c2(XP_006721028) c3(voltage-dependent T-type calcium channel subunit alpha-1H isoform X4) c4( ) c5(293821) c6( ) c7(ae, Cq, dD, s, xZ, ha, HN, aT, cz, ayD, eq, gu, hg, rz, Fg, ayC, ad, bN); #c1(CACNA1H) c2(XP_011521028) c3(voltage-dependent T-type calcium channel subunit alpha-1H isoform X10) c4( ) c5(315972) c6( ) c7(ae, Cq, dD, s, xZ, ha, HN, aT, cz, ayD, eq, gu, hg, rz, Fg, ayC, ad, bN); #c1(CACNA11) c2(NP_066919) c3(voltage-dependent T-type calcium channel subunit alpha-1H isoform a) c4(207457, 207458, 207459) c5(207460, 207461, 207462, 207463, 207464, 207465, 207466, 207467, 207468, 207469, 207470, 207471, 207472, 207473, 207474, 207475, 207476, 207477) c6( ) c7(pN); #c1 (CACNA11) c2(XP_011528783) c3(voltage-dependent T-type calcium channel subunit alpha-1l isoform X1) c4(324092, 324093, 324094) c5(324095, 324096, 324097, 324098, 324099, 324100, 324101, 324102, 324103, 324104, 324105, 324106, 324107, 324108, 324109, 324110, 324111, 324/12) c6( ) c7(pN); #c1(CACNA1S) c2(NP_000060) c3(voltage-dependent L-type calcium channel subunit alpha-IS) c4(1080) c5(1081, 1082, 1083, 1084, 1085, 1086) c6( ) c7(IJ, DT, GY, c, lg, ayl, dR, vj, xs, ayH, cV, lW, akB, ayr, ayG, ayE, cd, HT, ayF); #c1(CACNA1S) c2(XP_005245535) c3(voltage-dependent L-type calcium channel subunit alpha-1S isoform X1) c4(266697) c5(266698, 266699, 266700, 266701, 266702, 266703) c6( ) c7(IJ, DT, GY, c, lg, ayl, dR, vj, xs, ayH, cV, lW, akB, ayr, ayG, ayE, cd, HT, ayF); #c1(CACNA2D1) c2(NP_000713) c3(voltage-dependent calcium channel subunit alpha-2/delta-1 preproprotein) c4(14788) c5(14789, 14790, 14791, 14792, 14793, 14794) c6( ) c7(alC, ms, lW, oe, cV, ajM, j, fq, X); #c1(CACNA2D1) c2(XP_005250627) c3(voltage-dependent calcium channel subunit alpha-2/delta-l isoform X4) c4(270468) c5(270469, 270470, 270471, 270472, 270473, 270474) c6( ) c7(alC, ms, lW, oe, cV, ajM, j, fq, X); #c1(CACNA2D1) c2(XP_006716181) c3(voltage-dependent calcium channel subunit alpha-2/delta-1 isoform X1) c4(290136) c5(290137, 290138, 290139, 290140, 290141, 290142) c6( ) c7(alC, ms, lW, oe, cV, ajM, j, fq, X); #c1(CACNA2D1) c2(XP_006716183) c3(voltage-dependent calcium channel subunit alpha-2/delta-1 isoform X10) c4(290143) c5( ) c6( ) c7(alC, ms, lW, oe, cV, ajM, j, fq, X); #c1(CACNA2D1) c2(XP_006716184) c3(voltage-dependent calcium channel subunit alpha-2/delta-1 isoform X12) c4( ) c5(290144, 290145, 290146, 290147, 290148, 290149, 290150) c6( ) c7(alC, ms, lW, oe, cV, ajM, j, fq, X); #c1(CACNA202) c2(NP_001005505) c3(voltage-dependent calcium channel subunit alpha-2/delta-2 isoform a precursor) c4(21802, 21803) c5(21804, 21805, 21806, 21807, 21808, 21809, 21810, 21811, 21812, 21813, 21814, 21815) c6( ) c7(av, ky, eM, dB, aW, HN, aT, ayJ, lm, qN, bz, eA); #c1(CACNA2D2) c2(NP_001167522) c3(voltage-dependent calcium channel subunit alpha-2/delta-2 isoform c precursor) c4(68994) c5(68995, 68996, 68997, 68998, 68999, 69000) c6( ) c7(av, ky, eM, dB, aW, HN, aT, ayJ, lm, qN, bz, eA); #c1(CACNA2D2) c2(NP_001278030) c3(voltage-dependent calcium channel subunit alpha-2/delta-2 isoform d) c4(109420) c5(109421, 109422, 109423, 109424, 109425, 109426) c6( ) c7(av, ky, eM, dB, aW, HN, aT, ayJ, lm, qN, bz, eA); #c1(CACNA2D2) c2(NP_006021) c3(voltage-dependent calcium channel subunit alpha-2/delta-2 isoform b precursor) c4(161225) c5(16/226, 161227, 161228, 161229, 161230, 161231) c6( ) c7(av, ky, eM, dB, aW, HN, aT, ayJ, lm, qN, bz, eA); #c1(CACNA2D2) c2(XP_011532544) c3(voltage-dependent calcium channel subunit alpha-2/delta-2 isoform X1) c4(328641) c5(328642, 328643, 328644, 328645, 328646, 328647) c6( ) c7(av, ky, eM, dB, aW, HN, aT, ayJ, lm, qN, bz, eA); #c1(CACNA2D3) c2(NP_060868) c3(voltage-dependent calcium channel subunit alpha-2/delta-3 precursor) c4(198177, 198178, 198179) c5(198180, 198181, 198182, 198183, 198184, 198185, 198186, 198187, 198188, 198189, 198190, 198191, 198192, 198193, 198194, 198195, 198196, 198197, 198198, 198199, 198200, 198201, 198202, 198203, 198204, 198205) c6( ) c7(T, dY, aW, aw, aT, aA, aF, it, ayL, ayK); #c1(CACNA2D3) c2(XP_011532248) c3(voltage-dependent calcium channel subunit alpha-2/delta-3 isoform X2) c4(328308) c5( ) c6( ) c7(T, dY, aW, aw, aT, aA, aF, it, ayL, ayK); #c1(CACNA2D3) c2(XP_011532253) c3(voltage-dependent calcium channel subunit alpha-2/delta-3 isoform X7) c4(328309) c5(328310, 328311, 328312, 328313, 328314, 328315) c6( ) c7(T, dY, aW, aw, aT, aA, aF, it, ayL, ayK); #c1(CACNA2D3) c2(XP_011532255) c3(voltage-dependent calcium channel subunit alpha-2/delta-3 isoform X9) c4(328316) c5(328317, 328318, 328319, 328320, 328321, 328322, 328323, 328324, 328325, 328326, 328327, 328328) c6( ) c7(T, dY, aW, aw, aT, aA, aF, it, ayL, ayK); #c1(CACNA2D4) c2(NP_758952) c3(voltage-dependent calcium channel subunit alpha-2/delta-4) c4(249356, 249357, 249358, 249359, 249360) c5(249361, 249362, 249363, 249364, 249365, 249366, 249367, 249368, 249369, 249370, 249371, 249372, 249373, 249374, 249375, 249376, 249377, 249378, 249379, 249380, 249381, 249382, 249383, 249384, 249385, 249386, 249387, 249388, 249389, 249390, 249391, 249392, 249393, 249394, 249395, 249396, 249397) c6( ) c7(IR, ayM, uJ, bK, he, ayj); #c1(CACNB1) c2(NP_000714) c3(voltage-dependent L-type calcium channel subunit beta-1 isoform 1) c4(14795, 14796) c5(14797, 14798, 14799, 14800, 14801, 14802, 14803, 14804, 14805, 14806, 14807, 14808) c6( ) c7(aT); #c1(CACNB1) c2(NP_954855) c3(voltage-dependent L-type calcium channel subunit beta-1 isoform 2) c4(261407) c5(261408, 261409, 261410, 261411, 261412, 261413) c6( ) c7(aT); #c1(CACNB1) c2(NP_954856) c3(voltage-dependent L-type calcium channel subunit beta-1 isoform 3) c4(261414, 261415) c5(261416, 261417, 261418, 261419, 261420, 261421, 261422, 261423, 261424, 261425, 261426, 261427) c6( ) c7(aT); #c1(CACNB2) c2(NP_000715) c3(voltage-dependent L-type calcium channel subunit beta-2 isoform 1) c4(14809) c5(14810, 14811, 14812, 14813, 14814, 14815) c6( ) c7(cF, ac, rz, ms, oe, aA, cz, ayN, ajM, bK, vo, bN); #c1(CACNB2) c2(NP_963864) c3(voltage-dependent L-type calcium channel subunit beta-2 isoform 7) c4(262458) c5(262459, 262460, 262461, 262462, 262463, 262464) c6( ) c7(cF, ac, rz, ms, oe, aA, cz, ayN, ajM, bK, vo, bN); #c1(CACNB2) c2(NP_963865) c3(voltage-dependent L-type calcium channel subunit beta-2 isoform 6) c4(262465) c5(262466, 262467, 262468, 262469, 262470, 262471) c6( ) c7(cF, ac, rz, ms, oe, aA, cz, ayN, ajM, bK, vo, bN); #c1(CACNB2) c2(NP_963866) c3(voltage-dependent L-type calcium channel subunit beta-2 isoform 8) c4(262472) c5(262473, 262474, 262475, 262476, 262477, 262478) c6( ) c7(cF, ac, rz, ms, oe, aA, cz, ayN, ajM, bK, vo, bN); #c1(CACNB2) c2(NP_963884) c3(voltage-dependent L-type calcium channel subunit beta-2 isoform 3) c4(262496) c5(262497, 262498, 262499, 262500, 262501, 262502) c6( ) c7(cF, ac, rz, ms, oe, aA, cz, ayN, ajM, bK, vo, bN); #c1(CACNB2) c2(NP_963890) c3(voltage-dependent L-type calcium channel subunit beta-2 isoform 2) c4(262542) c5(262543, 262544, 262545, 262546, 262547, 262548) c6( ) c7(cF, ac, rz, ms, oe, aA, cz, ayN, ajM, bK, vo, bN); #c1(CACNB2) c2(NP_963891) c3(voltage-dependent L-type calcium channel subunit beta-2 isoform 4) c4(262549) c5(262550, 262551, 262552, 262553, 262554, 262555) c6( ) c7(cF, ac, rz, ms, oe, aA, cz, ayN, ajM, bK, vo, bN); #c1(CACNB2) c2(XP_011517962) c3(voltage-dependent L-type calcium channel subunit beta-2 isoform X4) c4(312001) c5(312002, 312003, 312004, 312005, 312006, 312007) c6( ) c7(cF, ac, rz, ms, oe, aA, cz, ayN, ajM, bK, vo, bN); #c1(CACNB3) c2(NP_000716) c3(voltage-dependent L-type calcium channel subunit beta-3 isoform 1) c4(14816) c5(14817, 14818, 14819, 14820, 14821, 14822) c6( ) c7(bL, bK, mu); #c1(CACNB3) c2(NP_001193844) c3(voltage-dependent L-type calcium channel subunit beta-3 isoform 2) c4(82710) c5(82711, 82712, 82713, 82714, 82715, 82716) c6( ) c7(bL, bK, mu); #c1(CACNB3) c2(NP_001193845) c3(voltage-dependent L-type calcium channel subunit beta-3 isoform 3) c4(82717) c5(82718, 82719, 82720, 82721, 82722, 82723) c6( ) c7(bL, bK, mu); #c1(CACNB3) c2(NP_001193846) c3(voltage-dependent L-type calcium channel subunit beta-3 isoform 4) c4(82724) c5(82725, 82726, 82727, 82728, 82729, 82730) c6( ) c7(bL, bK, mu); #c1(CACNB4) c2(NP_000717) c3(voltage-dependent L-type calcium channel subunit beta-4 isoform b) c4(14823, 14824) c5(14825, 14826, 14827, 14828, 14829, 14830, 14831, 14832, 14833, 14834, 14835, 14836) c6( ) c7(yr, Cq, XK, ayO, ha, iD, hg, akr); #c1(CACNB4) c2(NP_001005746) c3(voltage-dependent L-type calcium channel subunit beta-4 isoform c) c4(22023) c5(22024, 22025, 22026, 22027, 22028, 22029) c6( ) c7(yr, Cq, XK, ayO, ha, iD, hg, akr); #c1(CACNB4) c2(NP_001005747) c3(voltage-dependent L-type calcium channel subunit beta-4 isoform a) c4(22030, 22031) c5(22032, 22033, 22034, 22035, 22036, 22037, 22038, 22039, 22040, 22041, 22042, 22043) c6( ) c7(yr, Cq, XK, ayO, ha, iD, hg, akr); #c1(CACNB4) c2(NP_001139270) c3(voltage-dependent L-type calcium channel subunit beta-4 isoform d) c4(59059, 59060) c5(59061, 59062, 59063, 59064, 59065, 59066, 59067, 59068, 59069, 59070, 59071, 59072) c6( ) c7(yr, Cq, XK, ayO, ha, iD, hg, akr); #c1(CACNG2) c2(NP_006069) c3(voltage-dependent calcium channel gamma-2 subunit) c4(161649) c5(161650, 161651, 161652, 161653, 161654, 161655, 161656, 161657, 161658, 161659, 161660, 161661, 161662, 161663, 161664, 161665) c6( ) c7(alP, T, fX, ayP, Fo, ha, bk); #c1(CACNG3) c2(NP_006530) c3(voltage-dependent calcium channel gamma-3 subunit) c4(166044) c5(166045, 166046, 166047, 166048, 166049, 166050, 166051, 166052) c6( ) c7(W, HN, ha, ayQ, j); #c1(CACNG4) c2(NP_055220) c3(voltage-dependent calcium channel gamma-4 subunit) c4(179912) c5(179913, 179914, 179915, 179916, 179917, 179918) c6( ) c7(V); #c1(CACNG5) c2(XP_011522915) c3(voltage-dependent calcium channel gamma-5 subunit isoform X1) c4(317401, 317402) c5(317403, 317404, 317405, 317406, 317407, 317408, 317409, 317410, 317411, 317412, 317413, 317414, 317415, 317416, 317417, 317418, 317419, 317420) c6( ) c7(ayR); #c1(CACNG6) c2(NP_114103) c3(voltage-dependent calcium channel gamma-6 subunit isoform c) c4(222162) c5(222163, 222164, 222165, 222166, 222167, 222168) c6( ) c7(bM); #c1(CACNG6) c2(NP_665813) c3(voltage-dependent calcium channel gamma-6 subunit isoform a) c4(241748) c5(241749, 24/750, 24/751, 24/752, 241753, 24/754) c6( ) c7(bM); #c1(CACNG6) c2(NP_665814) c3(voltage-dependent calcium channel gamma-6 subunit isoform b) c4(24/755) c5(24/756, 241757, 241758, 241759, 241760, 24/761) c6( ) c7(bM); #c1(CACUL1) c2(NP_722517) c3(CDK2-associated and cullin domain-containing protein 1) c4(246758, 246759, 246760) c5(246761, 246762, 246763, 246764, 246765, 246766, 246767, 246768, 246769, 246770, 246771, 246772, 246773, 246774, 246775, 246776, 246777, 246778) c6( ) c7(bM, T, af, AM, aW, apx, aT, nf, r, aA, aF, fd, K, qN, g, q, dx, cn); #c1(CACUL1) c2(XP_011537650) c3(CDK2-associated and cullin domain-containing protein 1 isoform X1) c4(335209) c5(335210, 335211, 335212, 335213, 335214, 335215) c6( ) c7(bM, T, af, AM, aW, apx, aT, nf, r, aA, aF, fd, K, qN, g, q, dx, cn); #c1(CACYBP) c2(NP_001007215) c3(calcyclin-binding protein isoform 2) c4( ) c5(22558, 22559, 22560, 22561, 22562, 22563, 22564, 22565, 22566, 22567, 22568, 22569, 22570, 22571) c6( ) c7(es, aC, cc, aE, iA, bE, aW, aw, aA, bB, aT, aF, fr, eA); #c1(CACYBP) c2(NP_055227) c3(calcyclin-binding protein isoform 1) c4(179938, 179939, 179940, 179941, 179942) c5(179943, 179944, 179945, 179946, 179947, 179948, 179949, 179950, 179951, 179952, 179953, 179954, 179955, 179956, 179957, 179958, 179959, 179960, 179961, 179962) c6( ) c7(es, aC, cc, aE, iA, bE, aW, aw, aA, bB, aT, aF, fr, eA); #c1(CAD) c2(NP_004332) c3(CAD protein) c4(145221) c5(145222, 145223, 145224, 145225, 145226, 145227) c6( ) c7(bL, T, dD, ZV, Zz, jp, qb, ea, e, aw, cz, sM, vr, bS, j, ct, eq, zP, q, ad, av, ec, rS, aT, h, j, bq, de, eN, hm, CK, bf, cW, aW, Zt, r, aq, Z, VJ, ac, nB, wE, aka, ayS, vb, aS, pw, HE, B); #c1(CADM1) c2(NP_001091987) c3(cell adhesion molecule 1 isoform E precursor) c4(39150, 39151) c5(39152, 39153, 39154, 39155, 39156, 39157, 39158, 39159, 39160, 39161, 39162, 39163) c6( ) c7(T, sw, if, iW, WT, ex, jh, aA, n, Gn, ct, C, ci, nA, qN, q, ad, eA, eJ, dP, ip, av, ft, ib, aE, iu, cF, akU, rA, kH, aT, eV, dL, K, rz, eL, jf, ayT, je, iZ, aW, Y, ff, ayU, r, aq, Ej, kp, en, bu, g, cm, akw, bz, iR, bN, iM, Dy, eM, Ew, WU, Zn, adg, bt, it, B, ayV); #c1(CADM1) c2(NP_001287973) c3(cell adhesion molecule 1 isoform B precursor) c4(113292) c5(113293) c6( ) c7(T, sw, if, iW, WT, ex, jh, aA, n, Gn, ct, C, ci, nA, qN, q, ad, eA, eJ, dP, ip, av, ft, ib, aE, iu, cF, akU, rA, kH, aT, eV, dL, K, rz, eL, jf, ayT, je, iZ, aW, Y, ff, ayU, r, aq, Ej, kp, en, bu, g, cm, akw, bz, iR, bN, iM, Dy, eM, Ew, WU, Zn, adg, bt, it, B, ayV); #c1(CADM1) c2(NP_055148) c3(cell adhesion molecule 1 isoform D precursor) c4(179449) c5(179450, 179451, 179452, 179453, 179454, 179455) c6( ) c7(T, sw, if, iW, WT, ex, jh, aA, n, Gn, ct, C, ci, nA, qN, q, ad, eA, eJ, dP, ip, av, ft, ib, aE, iu, cF, akU, rA, kH, aT, eV, dL, K, rz, eL, jf, ayT, je, iZ, aW, Y, ff, ayU, r, aq, Ej, kp, en, bu, g, cm, akw, bz, iR, bN, iM, Dy, eM, Ew, WU, Zn, adg, bt, it, B, ayV); #c1(CADM1) c2(XP_005271551) c3(cell adhesion molecule 1 isoform X1) c4( ) c5(283468, 283469, 283470, 283471, 283472) c6( ) c7(T, sw, if, iW, WT, ex, jh, aA, n, Gn, ct, C, ci, nA, qN, q, ad, eA, eJ, dP, ip, av, ft, ib, aE, iu, cF, akU, rA, kH, aT, eV, dL, K, rz, eL, jf, ayT, je, iZ, aW, Y, ff, ayU, r, aq, Ej, kp, en, bu, g, cm, akw, bz, iR, bN, iM, Dy, eM, Ew, WU, Zn, adg, bt, it, B, ayV); #c1 (CADM2) c2(NP_001161146) c3(cell adhesion molecule 2 isoform 1 precursor) c4(65376) c5(65377, 65378, 65379, 65380, 65381, 65382) c6( ) c7(ae, T, hb, n, Jd, j, v, iV, fq, B, ad); #c1(CADM2) c2(NP_001161147) c3(cell adhesion molecule 2 isoform 2 precursor) c4(65383) c5(65384, 65385, 65386, 65387, 65388, 65389) c6( ) c7(ae, T, hb, n, Jd, j, v, iV, fq, B, ad); #c1(CADM2) c2(NP_694854) c3(cell adhesion molecule 2 isoform 3 precursor) c4(245390) c5(245391, 245392, 245393, 245394, 245395, 245396) c6( ) c7(ae, T, hb, n, Jd, j, v, iV, fq, B, ad); #c1(CADM3) c2(NP_001120645) c3(cell adhesion molecule 3 isoform 2 precursor) c4(44337, 44338) c5(44339, 44340, 44341, 44342, 44343, 44344, 44345, 44346, 44347, 44348, 44349, 44350) c6( ) c7(T, ae, eA, ad); #c1(CADM3) c2(NP_067012) c3(cell adhesion molecule 3 isoform 1 precursor) c4(208351) c5(208352, 208353, 208354, 208355, 208356, 208357) c6( ) c7(T, ae, eA, ad); #c1(CADM4) c2(NP_660339) c3(cell adhesion molecule 4 precursor) c4(241007) c5(241008, 241009, 241010, 241011, 241012, 241013) c6( ) c7(av, T, py, ae, eA, ad); #c1(CADPS) c2(NP_003707) c3(calcium-dependent secretion activator 1 isoform 1) c4(140312, 140313) c5(140314, 140315, 140316, 140317, 140318, 140319, 140320, 140321, 140322, 140323, 140324, 140325) c6( ) c7(ayW, bX, hH, ae, af, ad); #c1(CADPS) c2(NP_899630) c3(calcium-dependent secretion activator 1 isoform 3) c4(258075) c5(258076, 258077, 258078, 258079, 258080, 258081) c6( ) c7(ayW, bX, hH, ae, af, ad); #c1(CADPS) c2(NP_899631) c3(calcium-dependent secretion activator 1 isoform 2) c4(258082) c5(258083, 258084, 258085, 258086, 258087, 258088) c6( ) c7(ayW, bX, hH, ae, af, ad); #c1(CADPS) c2(XP_011532484) c3(calcium-dependent secretion activator 1 isoform X12) c4(328627) c5( ) c6( ) c7(ayW, bX, hH, ae, af, ad); #c1(CADPS) c2(XP_011532503) c3(calcium-dependent secretion activator 1 isoform X32) c4( ) c5(328628, 328629, 328630, 328631, 328632, 328633) c6( ) c7(ayW, bX, hH, ae, af, ad); #c1(CADPS2) c2(NP_001009571) c3(calcium-dependent secretion activator 2 isoform b) c4(23631, 23632, 23633) c5(23634, 23635, 23636, 23637, 23638, 23639, 23640, 23641, 23642, 23643, 23644, 23645, 23646, 23647, 23648, 23649, 23650, 23651) c6( ) c7(ac, rz, hb, ha, nA, he, lB, bN); #c1(CADPS2) c2(NP_001161412) c3(calcium-dependent secretion activator 2 isoform c) c4(65777) c5(65778, 65779, 65780, 65781, 65782, 65783) c6( ) c7(ac, rz, hb, ha, nA, he, lB, bN); #c1(CADPS2) c2(NP_060424) c3(calcium-dependent secretion activator 2 isoform a) c4(195891, 195892) c5(195893, 195894, 195895, 195896, 195897, 195898, 195899, 195900, 195901, 195902, 195903, 195904, 195905, 195906, 195907, 195908, 195909) c6( ) c7(ac, rz, hb, ha, nA, he, lB, bN); #c1(CADPS2) c2(XP_005250755) c3(calcium-dependent secretion activator 2 isoform X4) c4(270518) c5( ) c6( ) c7(ac, rz, hb, ha, nA, he, lB, bN); #c1(CADPS2) c2(XP_005250757) c3(calcium-dependent secretion activator 2 isoform X6) c4(270519) c5(270520, 270521, 270522, 270523, 270524, 270525) c6( ) c7(ac, rz, hb, ha, nA, he, lB, bN); #c1(CADPS2) c2(XP_005250759) c3(calcium-dependent secretion activator 2 isoform X8) c4( ) c5(270526) c6( ) c7(ac, rz, hb, ha, nA, he, lB, bN); #c1(CADPS2) c2(XP_005250765) c3(calcium-dependent secretion activator 2 isoform X17) c4(270527) c5(270528, 270529, 270530, 270531, 270532, 270533) c6( ) c7(ac, rz, hb, ha, nA, he, lB, bN); #c1(CAGE1) c2(NP_001164163) c3(cancer-associated gene 1 protein isoform 1) c4(66657, 66658) c5(66659, 66660, 66661, 66662, 66663, 66664, 66665, 66666, 66667, 66668, 66669, 66670, 66671, 66672, 66673, 66674, 66675, 66676, 66677) c6( ) c7(ci, g); #c1(CAGE1) c2(NP_001164164) c3(cancer-associated gene 1 protein isoform 2) c4(66678, 66679) c5(66680, 66681, 66682, 66683, 66684, 66685) c6( ) c7(ci, g); #c1(CAGE1) c2(NP_995586) c3(cancer-associated gene 1 protein isoform 3) c4(263971) c5(263972, 263973, 263974, 263975, 263976) c6( ) c7(ci, g); #c1(CALB1) c2(NP_004920) c3(calbindin) c4(150794, 150795) c5(150796, 150797, 150798, 150799, 150800, 150801, 150802, 150803, 150804, 150805, 150806, 150807) c6( ) c7(jf, es, rR, c, pY, hC, U, lb, n, dy, gn, bk, an, fr, he, nJ, af); #c1(CALB2) c2(NP_001731) c3 (calretinin isoform 1) c4(120579) c5(120580, 120581, 120582, 120583, 120584, 120585) c6( ) c7(T, arY, ib, nO, bE, ev, ayX, bO, r, bB, q, ci, PJ, g, Nm, ayY, qW, X, hS); #c1(CALB2) c2(NP_009019) c3 (calretinin isoform 22k) c4(170289) c5(170290, 170291, 170292, 170293, 170294, 170295) c6( ) c7(T, arY, ib, nO, bE, ev, ayX, bQ, r, bB, q, ci, PJ, g, Nm, ayY, qW, X, hS); #c1(CALCA) c2(NP_001029125) c3(calcitonin gene-related peptide 1 isoform alpha-CGRP preproprotein) c4(30764) c5(30765, 30766, 30767, 30768, 30769, 30770, 30771, 30772, 30773, 30774) c6( ) c7(oO, T, vS, fR, azf, azi, aza, fd, ci, aR, ip, x, Vp, azb, EL, H, azh, aW, g, eE, eF, uC, cm, bw, eM, nB, iA, iT, TG, cj, QF, azg, m, k, cZ, gp, My, yG, zt, n, aj, nA, ml, ai, ft, Do, azj, cU, dd, he, aze, A, sp, OX, AQ, zZ, vo, ar, kT, ok, Qy, iO, cA, B, bL, zm, fG, cz, adi, hz, v, C, AO, pd, rd, q, J, er, mj, ga, nv, akU, Qg, aT, cy, dz, bK, UN, bq, an, yj, ahE, ae, bA, hM, wr, s, iU, fO, r, ahl, en, t, f, bN, EJ, ck, alC, azc, Gg, bt, VB, ayZ, vi, df, ha, ml, jh, Hn, azd, uu, hF, ad, iC, av, hd, Rq, Aa, ji, yh, nJ, Yi, DI, aus, et, ff, eH, eZ, Mz, la, Sm, iV, HI, bz, dr, qa, es, D, yE, bE, rH, lC, X); #c1(CALCA) c2(NP_001732) c3(calcitonin isoform CT preproprotein) c4(120586, 120587, 120588) c5(120589, 120590, 120591, 120592, 120593, 120594, 120595, 120596, 120597, 120598, 120599, 120600, 120601, 120602, 120603, 120604, 120605, 120606, 120607, 120608, 120609, 120610) c6( ) c7(oO, T, vS, fR, azf, azi, aza, fd, ci, aR, ip, x, Vp, azb, EL, H, azh, aW, g, eE, eF, uC, cm, bw, eM, nB, iA, lT, TG, cj, QF, azg, m, k, cZ, gp, My, yG, zt, n, aj, nA, ml, ai, ft, Do, azj, cU, dd, he, aze, A, sp, OX, AQ, zZ, vo, ar, kT, ok, Qy, iO, cA, B, bL, zm, fG, cz, adi, hz, v, C, AO, pd, rd, q, J, er, mj, ga, nv, akU, Qg, aT, cy, dz, bK, UN, bq, an, yj, ahE, a, bA, hM, wr, s, iU, fO, r, ahl, en, t, f, bN, EJ, ck, alC, azc, Gg, bt, VB, ayZ, vi, df, ha, ml, jh, Hn, azd, uu, hF, ad, iC, av, hd, Rq, Aa, ji, yh, nJ, Yi, DI, aus, et, ff, eH, eZ, Mz, la, Sm, iV, HI, bz, dr, qa, es, D, yE, bE, rH, lC, X); #c1(CALCB) c2(NP_000719) c3(calcitonin gene-related peptide 2 precursor) c4(14837, 14838) c5(14839, 14840, 14841, 14842, 14843, 14844, 14845, 14846, 14847, 14848, 14849, 14850, 14851, 14852, 14853, 14854, 14855, 14856, 14857, 14858, 14859, 14860) c6( ) c7(nv); #c1(CALCOCO1) c2(NP_001137154) c3(calcium-binding and coiled-coil domain-containing protein 1 isoform 2) c4(55053) c5(55054, 55055, 55056, 55057, 55058, 55059) c6( ) c7(ww, cd, Fq, aT); #c1(CALCOCO1) c2(NP_065949) c3(calcium-binding and coiled-coil domain-containing protein 1 isoform 1) c4(206429, 206430, 206431, 206432) c5(206433, 206434, 206435, 206436, 206437, 206438, 206439, 206440, 206441, 206442, 206443, 206444, 206445, 206446, 206447, 206448, 206449, 206450, 206451, 206452, 206453, 206454, 206455, 206456) c6( ) c7(ww, cd, Fq, aT); #c1(CALCOCO1) c2(XP_011536906) c3(calcium-binding and coiled-coil domain-containing protein 1 isoform X1) c4(334572, 334573) c5(334574, 334575, 334576, 334577, 334578, 334579, 334580, 334581, 334582, 334583, 334584, 334585) c6( ) c7(ww, cd, Fq, aT); #c1(CALCOCO2) c2(NP_001248319) c3(calcium-binding and coiled-coil domain-containing protein 2 isoform 1) c4(93672) c5(93673, 93674, 93675, 93676, 93677, 93678) c6( ) c7(cm, BF, lo, bc); #c1(CALCOCO2) c2(NP_005822) c3(calcium-binding and coiled-coil domain-containing protein 2 isoform 3) c4(159706) c5(159707, 159708, 159709, 159710, 159711, 159712) c6( ) c7(cm, BF, lo, bc); #c1(CALCOCO2) c2(XP_011522483) c3(calcium-binding and coiled-coil domain-containing protein 2 isoform X1) c4(317120) c5(317121, 317122, 317123, 317124, 317125, 317126) c6( ) c7(cm, BF, lo, bc); #c1(CALCR) c2(NP_001158209) c3(calcitonin receptor isoform 1) c4(63353, 63354, 63355, 63356) c5(63357, 63358, 63359, 63360, 63361, 63362, 63363, 63364, 63365, 63366, 63367, 63368, 63369, 63370, 63371, 63372, 63373, 63374, 63375, 63376, 63377, 63378, 63379, 63380, 63381) c6( ) c7(T, zt, if, aw, NT, aA, bB, n, cl, fd, ml, qN, azk, cd, o, ad, eW, nv, aT, h, dL, K, ae, ake, af, bA, azh, eL, hM, Or, cH, aW, vj, nf, af, as, t, ve, bz, cm, bJ, eM, yE, at, uz, m); #c1 (CALCR) c2(NP_001158210) c3(calcitonin receptor isoform 2 precursor) c4(63382, 63383) c5(63384, 63385, 63386, 63387, 63388, 63389, 63390, 63391, 63392, 63393, 63394, 63395, 63396, 63397, 63398, 63399, 63400, 63401, 63402) c6( ) c7(T, zt, if, aw, NT, aA, bB, n, cl, fd, ml, qN, azk, cd, o, ad, eW, nv, aT, h, dL, K, ae, ake, af, bA, azh, eL, hM, Qr, cH, aW, vj, nf, aF, as, t, ve, bz, cm, bJ, eM, yE, at, uz, m); #c1(CALCRL) c2(NP_005786) c3 (calcitonin gene-related peptide type 1 receptor precursor) c4(159339, 159340) c5(159341, 159342, 159343, 159344, 159345, 159346, 159347, 159348, 159349, 159350, 159351, 159352, 159353, 159354, 159355, 159356, 159357, 159358, 159359, 159360, 159361) c6( ) c7(nJ, gx, T, d, aE, vj, fJ, sa, cz, cy, v, j, ci, m, azl, y, lJ, qa, bN); #c1(CALCRL) c2(XP_005246291) c3(calcitonin gene-related peptide type 1 receptor isoform X1) c4(267022) c5(267023, 267024, 267025, 267026, 267027) c6( ) c7(nJ, gx, T, d, aE, vj, fJ, sa, cz, cy, v, j, ci, m, azl, y, lJ, qa, bN); #c1(CALD1) c2(NP_004333) c3 (caldesmon isoform 2) c4(145228, 145229) c5( ) c6( ) c7(bL, T, dD, ZV, Zz, ea, e, aw, aA, sM, Bm, cz, bS, j, ct, bB, vr, eq, q, ad, eA, Zp, av, ec, hW, rS, oj, aT, h, By, K, jj, bq, dQ, pk, fb, eN, cY, hm, A, anR, bf, cW, aW, Zt, r, aF, Z, ac, nB, bE, wE, aka, vb, bo, dy, hE, B, y); #c1(CALD1) c2(NP_149129) c3(caldesmon isoform 1) c4(227348) c5( ) c6( ) c7(bL, T, dD, ZV, Zz, ea, e, aw, aA, sM, Bm, cz, bS, j, ct, bB, vr, eq, q, ad, eA, Zp, av, ec, hW, rS, oj, aT, h, By, K, jj, bq, dl, pk, fb, eN, cY, hm, A, anR, bf, cW, aW, Zt, r, aF, Z, ac, nB, bE, wE, aka, vb, bo, dy, hE, B, y); #c1(CALD1) c2(NP_149130) c3(caldesmon isoform 4) c4(227349) c5( ) c6( ) c7(bL, T, dD, ZV, Zz, ea, e, aw, aA, sM, Bm, cz, bS, j, ct, bB, vr, eq, q, ad, eA, Zp, av, ec, hW, rS, oj, aT, h, By, K, jj, bq, de, pk, fb, eN, cY, hm, A, anR, bf, cW, aW, Zt, r, aF, Z, ac, nB, bE, wE, aka, vb, bo, dy, hE, B, y); #c1(CALD1) c2(NP_149131) c3(caldesmon isoform 5) c4(227350, 227351) c5(227352, 227353, 227354, 227355, 227356, 227357, 227358, 227359, 227360, 227361, 227362, 227363, 227364, 227365, 227366, 227367, 227368, 227369, 227370, 227371, 227372, 227373, 227374, 227375, 227376, 227377, 227378, 227379, 227380, 227381, 227382, 227383, 227384, 227385, 227386, 227387, 227388, 227389, 227390, 227391) c6( ) c7(bL, T, dD, ZV, Zz, ea, e, aw, aA, sM, Bm, cz, bS, j, ct, bB, vr, eq, q, ad, eA, Zp, av, ec, hW, rS, oj, aT, h, By, K, jj, bq, dQ, pk, fb, eN, cY, hm, A, anR, bf, cW, aW, Zt, r, af, Z, ac, nB, bE, wE, aka, vb, bo, dy, HE, B, y); #c1(CALD1) c2(NP_149347) c3(caldesmon isoform 3) c4(227432, 227433) c5(227434) c6( ) c7(bL, T, dD, ZV, Zz, ea, e, aw, aA, sM, Bm, cz, bS, j, ct, bB, vr, eq, q, ad, eA, Zp, av, ec, hW, rS, oj, aT, h, By, K, jj, bq, dl, pk, fb, eN, cY, hm, A, anR, bf, cW, aW, Zt, r, aF, Z, ac, nB, bE, wE, aka, vb, bo, dy, hE, B, y); #c1(CALD1) c2(XP_006716202) c3(caldesmon isoform X3) c4( ) c5(290165, 290166, 290167, 290168, 290169, 290170, 290171, 290172, 290173, 290174, 290175, 290176, 290177, 290178, 290179, 290180, 290181, 290182, 290183, 290184, 290185, 290186, 290187, 290188, 290189, 290190, 290191) c6( ) c7(bL, T, dD, ZV, Zz, ea, e, aw, aA, sM, Bm, cz, bS, j, ct, bB, vr, eq, q, ad, eA, Zp, av, ec, hW, rS, oj, aT, h, By, K, jj, bq, dl, pk, fb, eN, cY, hm, A, anR, bf, cW, aW, Zt, r, aF, Z, ac, nB, bE, wE, aka, vb, bo, dy, hE, B, y); #c1(CALD1) c2(XP_011514887) c3(caldesmon isoform X2) c4(307638, 307639) c5( ) c6( ) c7(bL, T, dD, ZV, Zz, ea, e, aw, aA, sM, Bm, cz, bS, j, ct, bB, vr, eq, q, ad, eA, Zp, av, ec, hW, rS, oj, aT, h, By, K, jj, bq, dQ, pk, fb, eN, cY, hm, A, anR, bf, cW, aW, Zt, r, aF, Z, ac, nB, bE, wE, aka, vb, bo, dy, hE, B, y); #c1(CALHM1) c2(NP_001001412) c3(calcium homeostasis modulator protein 1) c4(18357) c5(18358, 18359, 18360, 18361, 18362, 18363) c6( ) c7(il, fk, af); #c1(CALHM2) c2(XP_006717946) c3(calcium homeostasis modulator protein 2 isoform X1) c4(291394, 291395) c5(291396, 291397, 291398, 291399, 291400, 291401, 291402, 291403, 291404, 291405, 291406, 291407, 291408, 291409, 291410, 291411) c6( ) c7(af); #c1(CALHM3) c2(NP_001123214) c3(calcium homeostasis modulator protein 3) c4(46836) c5(46837, 46838, 46839, 46840, 46841, 46842) c6( ) c7(af); #c1(CALM2) c2(NP_001734) c3(calmodulin) c4( ) c5(120611, 120612, 120613, 120614, 120615, 120616, 120617, 120618, 120619, 120620, 120621, 120622, 120623, 120624, 120625, 120626, 120627) c6( ) c7(bM, T, if, e, fd, ci, Sp, add, ip, aE, azm, K, eL, cp, eG, aW, lW, as, g, cm, eM, iA, iT, vA, U, cj, iS, m, xh, FG, fr, ct, qN, cd, ft, jz, h, dL, hB, azp, HO, CQ, mo, op, hq, ok, agl, azr, B, azo, bL, mr, rf, cz, arD, ajM, v, j, il, eA, azn, nv, aT, rO, XZ, ae, eu, bA, jf, s, iU, Y, Pg, ayr, cu, en, hf, t, ck, cl, ac, nM, gX, acl, gT, bt, ao, hl, qb, jh, rr, bS, hF, ad, Xv, ib, py, azq, io, vc, hP, mu, d, ff, gE, Z, jb, bz, qa, ek, es, bE, gZ, pK, lC, y); #c1(CALML3) c2(NP_005176) c3(calmodulin-like protein 3) c4(153643) c5(153644, 153645, 153646, 153647, 153648, 153649) c6( ) c7(Na, azs, amJ, eH, Nc, aml, eF); #c1(CALML5) c2(NP_059118) c3(calmodulin-like protein 5) c4(193135) c5(193136, 193137, 193138, 193139, 193140, 193141) c6( ) c7(IQ, cr); #c1(CALN1) c2(NP_001017440) c3(calcium-binding protein 8 isoform 2) c4(26522, 26523) c5(26524, 26525, 26526, 26527, 26528, 26529, 26530, 26531, 26532, 26533, 26534, 26535, 26536, 26537, 26538) c6( ) c7(aF, aA); #c1(CALN1) c2(NP_113656) c3(calcium-binding protein 8 isoform 1) c4(221964) c5(221965, 221966, 221967, 221968, 221969, 221970, 221971, 221972, 221973, 221974) c6( ) c7(aF, aA); #c1(CALN1) c2(XP_011514896) c3(calcium-binding protein 8 isoform X2) c4( ) c5(307657, 307658, 307659) c6( ) c7(aF, aA); #c1(CALN1) c2(XP_011514900) c3(calcium-binding protein 8 isoform X3) c4( ) c5(307660, 307661, 307662, 307663, 307664, 307665) c6( ) c7(aF, aA); #c1(CALR) c2(NP_004334) c3(calreticulin precursor) c4(145230, 145231) c5(145232, 145233, 145234, 145235, 145236, 145237, 145238, 145239, 145240, 145241, 145242, 145243, 145244) c6( ) c7(DS, T, if, al, eb, bB, qj, ci, xy, aE, rw, bW, pX, K, hu, azx, ake, eL, cp, azu, yo, up, aW, NR, g, as, cm, uC, mx, cj, m, NT, n, qN, cd, eW, azw, b, eT, azt, h, dL, he, agF, pm, cA, hR, pg, fL, it, B, iH, mr, aA, j, q, eA, MK, aT, yu, dz, bK, atZ, azv, ae, dl, s, oV, Y, r, ck, au, bt, vi, qi, mC, ad, yt, NS, HD, lQ, Yj, d, et, cH, afi, eZ, nf, gl, kD, yy, aF, YZ, bF, gk, pc, bE, azy, i, y, X, pC); #c1 (CALR3) c2(NP_659483) c3(calreticulin-3 precursor) c4(240165) c5(240166, 240167, 240168, 240169, 240170, 240171) c6( ) c7(azz); #c1(CALU) c2(NP_001124146) c3(calumenin isoform b precursor) c4(48269) c5(48270, 48271, 48272, 48273, 48274, 48275, 48276, 48277, 48278, 48279, 48280, 48281, 48282, 48283, 48284, 48285, 48286, 48287, 48288, 48289, 48290) c6( ) c7(T, dD, d, eM, yt, ea, bE, bB, j, lS, eq, aaD, iu); #c1(CALU) c2(NP_001186600) c3(calumenin isoform c precursor) c4(77918, 77919, 77920, 77921, 77922) c5(77923, 77924, 77925, 77926, 77927, 77928, 77929, 77930, 77931, 77932, 77933, 77934, 77935, 77936, 77937, 77938, 77939, 77940) c6( ) c7(T, dD, d, eM, yt, ea, bE, bB, j, lS, eq, aaD, iu); #c1 (CALU) c2(NP_001186601) c3(calumenin isoform d precursor) c4(77941, 77942, 77943, 77944, 77945) c5(77946, 77947, 77948, 77949, 77950, 77951, 77952, 77953, 77954, 77955, 77956) c6( ) c7(T, dD, d, eM, yt, ea, bE, bB, j, lS, eq, aaD, iu); #c1(CALU) c2(NP_001186602) c3(calumenin isoform e precursor) c4(77957) c5(77958, 77959, 77960, 77961, 77962, 77963, 77964, 77965, 77966, 77967, 77968, 77969, 77970, 77971, 77972) c6( ) c7(T, dD, d, eM, yt, ea, bE, bB, j, lS, eq, aaD, iu); #c1(CALU) c2(NP_001210) c3(calumenin isoform a precursor) c4( ) c5(83749, 83750, 83751, 83752, 83753, 83754, 83755, 83756, 83757, 83758, 83759, 83760, 83761, 83762) c6( ) c7(T, dD, d, eM, yt, ea, bE, bB, j, lS, eq, aaD, iu); #c1(CALU) c2(XP_011514890) c3(calumenin isoform X1) c4( ) c5(307640, 307641, 307642, 307643, 307644, 307645, 307646, 307647, 307648, 307649, 307650, 307651, 307652, 307653, 307654, 307655, 307656) c6( ) c7(T, dD, d, eM, yt, ea, bE, bB, j, lS, eq, aaD, iu); #c1(CALY) c2(NP_056537) c3(neuron-specific vesicular protein calcyon) c4(188007, 188008) c5(188009, 188010, 188011, 188012, 188013, 188014) c6( ) c7(Gx); #c1(CALY) c2(XP_011538141) c3(neuron-specific vesicular protein calcyon isoform X4) c4(335728) c5(335729, 335730, 335731, 335732, 335733, 335734, 335735, 335736, 335737, 335738, 335739, 335740, 335741, 335742, 335743, 335744) c6( ) c7(Gx); #c1(CAMK1) c2(NP_003647) c3(calcium/calmodulin-dependent protein kinase type 1) c4(139909) c5(139910, 139911, 139912, 139913, 139914, 139915) c6( ) c7(jf, eM, aW, aT, ae, ad); #c1(CAMK1D) c2(NP_065130) c3(calcium/calmodulin-dependent protein kinase type ID isoform 1) c4(204085) c5(204086, 204087, 204088, 204089, 204090, 204091) c6( ) c7(bL, Bl, T, cF, pN, aW, e, aT, h, aj, v, ad); #c1(CAMK1D) c2(NP_705718) c3(calcium/calmodulin-dependent protein kinase type ID isoform 2) c4(246282) c5(246283, 246284, 246285, 246286, 246287, 246288) c6( ) c7(bL, Bl, T, cF, pN, aW, e, aT, h, aj, v, ad); #c1(CAMK1G) c2(NP_065172) c3(calcium/calmodulin-dependent protein kinase type 1G) c4(204377) c5(204378, 204379, 204380, 204381, 204382, 204383, 204384, 204385, 204386, 204387, 204388, 204389) c6(204390, 204391) c7(c); #c1(CAMK2A) c2(NP_057065) c3(calcium/calmodulin-dependent protein kinase type II subunit alpha isoform 1) c4(188735, 188736) c5(188737, 188738, 188739, 188740, 188741, 188742) c6( ) c7(ff, Xg, cu, bK, en, nA, eF, af); #c1(CAMK2A) c2(NP_741960) c3(calcium/calmodulin-dependent protein kinase type II subunit alpha isoform 2) c4(247468) c5(247469, 247470, 247471, 247472, 247473, 247474, 247475, 247476, 247477, 247478, 247479, 247480) c6( ) c7(ff, Xg, cu, bK, en, nA, eF, af); #c1(CAMK2B) c2(NP_001211) c3(calcium/calmodulin-dependent protein kinase type II subunit beta isoform 1) c4(83763) c5(83764, 83765, 83766, 83767, 83768, 83769) c6( ) c7(Y, cu, sv, acI, ei, jb, eA, cp); #c1(CAMK2B) c2(NP_742075) c3(calcium/calmodulin-dependent protein kinase type II subunit beta isoform 2) c4(247968, 247969) c5(247970, 247971, 247972, 247973, 247974, 247975, 247976, 247977, 247978, 247979, 247980, 247981, 247982, 247983, 247984, 247985) c6( ) c7(Y, cu, sv, acI, ei, jb, eA, cp); #c1(CAMK2B) c2(NP_742076) c3(calcium/calmodulin-dependent protein kinase type II subunit beta isoform 3) c4(247986) c5(247987, 247988, 247989, 247990, 247991, 247992, 247993, 247994, 247995, 247996) c6( ) c7(Y, cu, sv, acI, ei, jb, eA, cp); #c1(CAMK2B) c2(NP_742077) c3(calcium/calmodulin-dependent protein kinase type 1 subunit beta isoform 4) c4(247997) c5(247998, 247999, 248000, 248001, 248002, 248003) c6( ) c7(Y, cu, sv, acI, ei, jb, eA, cp); #c1(CAMK2B) c2(NP_742078) c3(calcium/calmodulin-dependent protein kinase type II subunit beta isoform 5) c4(248004) c5(248005, 248006, 248007, 248008, 248009, 248010) c6( ) c7(Y, cu, sv, acI, ei, jb, eA, cp); #c1(CAMK2B) c2(NP_742079) c3(calcium/calmodulin-dependent protein kinase type II subunit beta isoform 6) c4(248011) c5(248012, 248013, 248014, 248015, 248016, 248017) c6( ) c7(Y, cu, sv, acI, ei, jb, eA, cp); #c1(CAMK2B) c2(NP_742080) c3(calcium/calmodulin-dependent protein kinase type II subunit beta isoform 7) c4(248018) c5(248019, 248020, 248021, 248022, 248023, 248024) c6( ) c7(Y, cu, sv, acI, ei, jb, eA, cp); #c1(CAMK2B) c2(NP_742081) c3(calcium/calmodulin-dependent protein kinase type II subunit beta isoform 8) c4(248025) c5(248026, 248027, 248028, 248029, 248030, 248031) c6( ) c7(Y, cu, sv, acI, ei, jb, eA, cp); #c1(CAMK2D) c2(NP_001212) c3(calcium/calmodulin-dependent protein kinase type II subunit delta isoform 3) c4(83770) c5(83771, 83772, 83773, 83774, 83775, 83776) c6( ) c7(kl, ac, ha, j, m, lf, cd); #c1(CAMK2D) c2(NP_742112) c3(calcium/calmodulin-dependent protein kinase type II subunit delta isoform 5) c4(248174) c5(248175, 248176, 248177, 248178, 248179, 248180) c6( ) c7(kl, ac, ha, j, m, lf, cd); #c1(CAMK2D) c2(NP_742113) c3(calcium/calmodulin-dependent protein kinase type II subunit delta isoform 1) c4(248181) c5(248182, 248183, 248184, 248185, 248186, 248187, 248188, 248189, 248190, 248191, 248192, 248193, 248194, 248195, 248196, 248197) c6( ) c7(kl, ac, ha, j, m, lf, cd); #c1(CAMK2D) c2(NP_742126) c3(calcium/calmodulin-dependent protein kinase type II subunit delta isoform 2) c4(248198) c5(248199, 248200, 248201, 248202, 248203, 248204) c6( ) c7(kl, ac, ha, j, m, lf, cd); #c1(CAMK2D) c2(NP_742127) c3(calcium/calmodulin-dependent protein kinase type II subunit delta isoform 4) c4(248205) c5(248206, 248207, 248208, 248209, 248210, 248211) c6( ) c7(kl, ac, ha, j, m, lf, cd); #c1(CAMK2D) c2(XP_005263308) c3(calcium/calmodulin-dependent protein kinase type II subunit delta isoform X3) c4(278394, 278395) c5(278396, 278397, 278398, 278399, 278400, 278401, 278402, 278403, 278404, 278405, 278406, 278407) c6( ) c7(kl, ac, ha, j, m, lf, cd); #c1(CAMK2D) c2(XP_005263312) c3(calcium/calmodulin-dependent protein kinase type II subunit delta isoform X7) c4(278408) c5(278409, 278410, 278411, 278412, 278413, 278414) c6( ) c7(kl, ac, ha, j, m, lf, cd); #c1(CAMK2D) c2(XP_006714393) c3(calcium/calmodulin-dependent protein kinase type II subunit delta isoform X15) c4(287826) c5( ) c6( ) c7(kl, ac, ha, j, m, lf, cd); #c1(CAMK2D) c2(XP_011530591) c3(calcium/calmodulin-dependent protein kinase type II subunit delta isoform X11) c4(326495) c5(326496, 326497, 326498, 326499, 326500, 326501) c6( ) c7(kl, ac, ha, j, m, lf, cd); #c1(CAMK2G) c2(NP_001213) c3(calcium/calmodulin-dependent protein kinase type II subunit gamma isoform 4) c4(83777) c5(83778, 83779, 83780, 83781, 83782, 83783) c6( ) c7(af, iT); #c1(CAMK2G) c2(NP_751909) c3(calcium/calmodulin-dependent protein kinase type II subunit gamma isoform 2) c4(248515) c5(248516, 248517, 248518, 248519, 248520, 248521) c6( ) c7(af, iT); #c1(CAMK2G) c2(NP_751910) c3(calcium/calmodulin-dependent protein kinase type II subunit gamma isoform 3) c4(248522) c5(248523, 248524, 248525, 248526, 248527, 248528) c6( ) c7(af, iT); #c1(CAMK2G) c2(NP_751911) c3(calcium/calmodulin-dependent protein kinase type II subunit gamma isoform 1) c4(248529, 248530) c5(248531, 248532, 248533, 248534, 248535, 248536, 248537, 248538, 248539, 248540, 248541, 248542, 248543) c6( ) c7(af, iT); #c1(CAMK2G) c2(XP_005270259) c3(calcium/calmodulin-dependent protein kinase type II subunit gamma isoform X16) c4(283301) c5(283302, 283303, 283304, 283305, 283306, 283307) c6( ) c7(af, iT); #c1(CAMK2G) c2(XP_005270260) c3(calcium/calmodulin-dependent protein kinase type II subunit gamma isoform X17) c4(283308) c5(283309, 283310, 283311, 283312, 283313, 283314) c6( ) c7(af, iT); #c1(CAMK2G) c2(XP_005270262) c3(calcium/calmodulin-dependent protein kinase type II subunit gamma isoform X18) c4(283315) c5(283316, 283317, 283318, 283319, 283320, 283321) c6( ) c7(af, iT); #c1(CAMK2G) c2(XP_006718056) c3(calcium/calmodulin-dependent protein kinase type II subunit gamma isoform X1) c4(291482) c5(291483, 291484, 291485, 291486, 291487, 291488) c6( ) c7(af, lT); #c1(CAMK2G) c2(XP_006718059) c3(calcium/calmodulin-dependent protein kinase type II subunit gamma isoform X6) c4(291489, 291490) c5(291491, 291492, 291493, 291494, 291495, 291496) c6( ) c7(af, iT); #c1(CAMK2G) c2(XP_011538527) c3(calcium/calmodulin-dependent protein kinase type II subunit gamma isoform X4) c4( ) c5(336284) c6( ) c7(af, iT); #c1(CAMK2G) c2(XP_011538533) c3(calcium/calmodulin-dependent protein kinase type II subunit gamma isoform X22) c4( ) c5(336285, 336286, 336287, 336288, 336289) c6( ) c7(af, iT); #c1(CAMK4) c2(NP_001735) c3(calcium/calmodulin-dependent protein kinase type IV) c4(120628, 120629) c5(120630, 120631, 120632, 120633, 120634, 120635, 120636, 120637, 120638, 120639, 120640, 120641, 120642, 120643, 120644, 120645) c6( ) c7(cF, ND, oe, XY, eZ, pk, vH, cu, fl, lQ, Ea, uE, t, mC, Hr, hq); #c1(CAMKK1) c2(NP_757343) c3(calcium/calmodulin-dependent protein kinase kinase 1 isoform a) c4(248619, 248620) c5(248621, 248622, 248623, 248624, 248625, 248626, 248627, 248628, 248629, 248630, 248631, 248632) c6( ) c7(T, bz, aR); #c1(CAMKK1) c2(XP_006721651) c3(calcium/calmodulin-dependent protein kinase kinase 1 isoform X2) c4(294140) c5(294141, 294142, 294143, 294144, 294145, 294146, 294147, 294148, 294149, 294150, 294151, 294152, 294153, 294154) c6(294155, 294156) c7(T, bz, aR); #c1(CAMKK1) c2(XP_011522333) c3(calcium/calmodulin-dependent protein kinase kinase 1 isoform X1) c4(317016) c5( ) c6( ) c7(T, bz, aR); #c1(CAMKK2) c2(NP_001257414) c3(calcium/calmodulin-dependent protein kinase kinase 2 isoform 1) c4(95390) c5(95391, 95392, 95393, 95394, 95395, 95396, 95397, 95398, 95399, 95400) c6( ) c7(dA, M, mf, bK, m, v, azA, t, ae, ad, bA, hq); #c1(CAMKK2) c2(NP_001257415) c3(calcium/calmodulin-dependent protein kinase kinase 2 isoform 7) c4(95401) c5(95402, 95403, 95404, 95405, 95406, 95407) c6( ) c7(dA, M, mf, bk, m, v, azA, t, ae, ad, bA, hq); #c1(CAMKK2) c2(NP_757364) c3(calcium/calmodulin-dependent protein kinase kinase 2 isoform 6) c4(248712) c5(248713, 248714, 248715, 248716, 248717, 248718, 248719, 248720, 248721, 248722, 248723, 248724, 248725, 248726, 248727, 248728, 248729, 248730, 248731, 248732, 248733, 248734, 248735) c6( ) c7(dA, M, mf, bK, m, v, azA, t, ae, ad, bA, hq); #c1(CAMKK2) c2(NP_757380) c3(calcium/calmodulin-dependent protein kinase kinase 2 isoform 2) c4(249088, 249089) c5(249090, 249091, 249092, 249093, 249094, 249095, 249096, 249097, 249098, 249099, 249100, 249101, 249102, 249103, 249104, 249105) c6( ) c7(dA, M, mf, bk, m, v, azA, t, ae, ad, bA, hq); #c1(CAMKK2) c2(XP_005253879) c3(calcium/calmodulin-dependent protein kinase kinase 2 isoform X3) c4(272751) c5(272752, 272753, 272754, 272755, 272756, 272757) c6( ) c7(dA, M, mf, bK, m, v, azA, t, ae, ad, bA, hq); #c1(CAMKK2) c2(XP_005253880) c3(calcium/calmodulin-dependent protein kinase kinase 2 isoform X1) c4( ) c5(272758, 272759, 272760, 272761, 272762, 272763) c6( ) c7(dA, M, mf, bK, m, v, azA, t, ae, ad, bA, hq); #c1(CAMKK2) c2(XP_005253881) c3(calcium/calmodulin-dependent protein kinase kinase 2 isoform X2) c4(272764) c5(272765, 272766, 272767, 272768, 272769, 272770) c6( ) c7(dA, M, mf, bK, m, v, azA, t, ae, ad, bA, hq); #c1(CAMKK2) c2(XP_011536064) c3(calcium/calmodulin-dependent protein kinase kinase 2 isoform X5) c4(333389, 333390, 333391, 333392) c5( ) c6( ) c7(dA, M, mf, bK, m, v, azA, t, ae, ad, bA, hq); #c1(CAMKMT) c2(NP_079042) c3(calmodulin-lysine N-methyltransferase) c4(216717) c5(216718, 216719, 216720, 216721, 216722, 216723) c6( ) c7(bM, T, qb, jh, fd, bS, fr, ct, ci, pK, eA, ad, ip, ft, ib, pN, azB, lC, aT, io, h, dL, bK, K, hB, ae, eL, cp, d, s, iU, aW, Y, ff, CO, en, Z, t, cm, azC, op, es, iA, vA, m, azr, xh, gT, B); #c1(CAMKMT) c2(XP_011531414) c3(calmodulin-lysine N-methyltransferase isoform X2) c4(327500) c5(327501, 327502, 327503, 327504, 327505, 327506) c6( ) c7(bM, T, qb, jh, fd, bS, fr, ct, ci, pK, eA, ad, ip, ft, ib, pN, azB, lC, aT, io, h, dL, bK, K, hB, ae, eL, cp, d, s, iU, aW, Y, ff, CO, en, Z, t, cm, azC, op, es, iA, vA, m, azr, xh, gT, B); #c1(CAMLG) c2(NP_001736) c3(calcium signal-modulating cyclophilin ligand) c4(120646) c5(120647, 120648, 120649, (20650, (20651, (20652) c6( ) c7(ck, T, dD, aW, Y, jh, aT, eq, bt); #c1(CAMP) c2(NP_004336) c3(cathelicidin antimicrobial peptide preproprotein) c4(145252, 145253, 145254, 145255, 145256, 145257, 145258, 145259, 145260, 145261, 145262, 145263, 145264, 145265, 145266, 145267, 145268, 145269, 145270, 145271, 145272, 145273, 145274, 145275, 145276, 145277, 145278, 145279, 145280, 145281, 145282, 145283, 145284) c5(145285, 145286, 145287, 145288, 145289, 145290, 145291, 145292, 145293, 145294, 145295, 145296, 145297, 145298, 145299, 145300, 145301, 145302, 145303, 145304, 145305, 145306, 145307, 145308, 145309, 145310, 145311, 145312, 145313, 145314, 145315, 145316, 145317, 145318, 145319, 145320, 145321, 145322, 145323, 145324, 145325, 145326, 145327, 145328, 145329, 145330, 145331, 145332, 145333, 145334, 145335, 145336, 145337, 145338, 145339, 145340, 145341, 145342, 145343, 145344, 145345, 145346, 145347, 145348, 145349, 145350, 145351, 145352, 145353, 145354, 145355, 145356, 145357, 145358, 145359, 145360, 145361, 145362, 145363, 145364, 145365, 145366, 145367, 145368, 145369, 145370, 145371, 145372, 145373, 145374, 145375, 145376, 145377, 145378, 145379, 145380, 145381, 145382, 145383, 145384, 145385, 145386, 145387, 145388, 145389, 145390, 145391, 145392, 145393, 145394, 145395, 145396, 145397, 145398, 145399, 145400, 145401, 145402, 145403, 145404, 145405, 145406, 145407, 145408, 145409, 145410, 145411, 145412, 145413, 145414, 145415, 145416, 145417, 145418, 145419, 145420, 145421, 145422, 145423, 145424, 145425, 145426, 145427, 145428, 145429, 145430, 145431, 145432, 145433, 145434, 145435, 145436, 145437, 145438, 145439, 145440, 145441, 145442, 145443, 145444, 145445, 145446, 145447, 145448, 145449, 145450, 145451, 145452, 145453, 145454, 145455, 145456, 145457, 145458, 145459, 145460, 145461, 145462, 145463, 145464, 145465, 145466, 145467) c6(145468, 145469, 145470, 145471) c7(oO, gV, ka, T, dD, dA, fR, bM, ea, azE, wh, aA, bB, afK, azF, BF, eq, C, pM, asS, ad, eA, x, av, hd, azD, aT, hD, h, lQ, DY, ae, aa, RT, XE, LC, aW, bg, cr, eZ, Kb, aF, g, ajz, t, f, bz, eE, zV, EJ, ck, ajl, ba, iT, Jr, xD, nB, ki, bE, mx, at, Zn, JH, k, m, bt, kS, ao); #c1(CAMSAP1) c2(NP_056262) c3(calmodulin-regulated spectrin-associated protein 1) c4(186791) c5(186792, 186793, 186794, 186795, 186796, 186797, 186798, 186799, 186800, 186801, 186802, 186803) c6( ) c7(Ej, T); #c1(CAMSAP1) c2(XP_005263453) c3(calmodulin-regulated spectrin-associated protein 1 isoform X1) c4(278477) c5(278478, 278479, 278480, 278481, 278482, 278483) c6( ) c7(Ej, T); #c1(CAMSAP1) c2(XP_011516574) c3 (calmodulin-regulated spectrin-associated protein 1 isoform X2) c4(310031) c5( ) c6( ) c7(Ej, T); #c1(CAMSAP2) c2(NP_001284636) c3(calmodulin-regulated spectrin-associated protein 2 isoform 1) c4(112198) c5(112199, 112200, 112201, 112202, 112203, 112204) c6( ) c7(ha); #c1(CAMSAP2) c2(NP_001284637) c3(calmodulin-regulated spectrin-associated protein 2 isoform 3) c4(112205) c5(112206, 112207, 112208, 112209, 112210, 112211) c6( ) c7(ha); #c1(CAMSAP2) c2(NP_982284) c3(calmodulin-regulated spectrin-associated protein 2 isoform 2) c4(263279) c5(263280, 263281, 263282, 263283, 263284, 263285) c6( ) c7(ha); #c1(CAMTA1) c2(XP_011539388) c3(calmodulin-binding transcription activator 1 isoform X4) c4(337493, 337494) c5(337495, 337496, 337497, 337498, 337499, 337500, 337501, 337502, 337503, 337504, 337505, 337506) c6( ) c7(azH, bL, d, ac, azG, j, eA, nA, cm, Fc, cd); #c1(CAMTA1) c2(XP_011539389) c3(calmodulin-binding transcription activator 1 isoform X5) c4(337507, 337508) c5( ) c6( ) c7(azH, bL, d, ac, azG, j, eA, nA, cm, Fc, cd); #c1(CAMTA1) c2(XP_011539390) c3(calmodulin-binding transcription activator 1 isoform X6) c4( ) c5(337509, 337510, 337511, 337512, 337513, 337514) c6( ) c7(azH, bL, d, ac, azG, j, eA, nA, cm, Fc, cd); #c1(CAND1) c2(NP_060918) c3(cullin-associated NEDD8-dissociated protein 1) c4(198616, 198617) c5(198618, 198619, 198620, 198621, 198622, 198623, 198624, 198625, 198626, 198627, 198628, 198629, 198630, 198631, 198632, 198633, 198634) c6( ) c7(T, g, Ea, ae, ad, bA); #c1(CAND2) c2(NP_001155971) c3(cullin-associated NEDD8-dissociated protein 2 isoform 1) c4(61666) c5(61667, 61668, 61669, 61670, 61671, 61672) c6( ) c7(XW); #c1(CAND2) c2(NP_036430) c3(cullin-associated NEDD8-dissociated protein 2 isoform 2) c4(173808) c5(173809, 173810, 173811, 173812, 173813, 173814) c6( ) c7(XW); #c1(CANT1) c2(XP_005257079) c3(soluble calcium-activated nucleotidase 1 isoform X1) c4(274661, 274662, 274663) c5(274664, 274665, 274666, 274667, 274668, 274669, 274670, 274671, 274672, 274673, 274674, 274675, 274676, 274677, 274678, 274679, 274680, 274681, 274682, 274683, 274684, 274685, 274686, 274687, 274688, 274689, 274690, 274691) c6( ) c7(dD, kB, KH, U, eq, azl, bC, kC, aR); #c1(CANX) c2(XP_011532966) c3(calnexin isoform X2) c4(329127, 329128, 329129, 329130, 329131, 329132, 329133, 329134, 329135, 329136, 329137) c5(329138, 329139, 329140, 329141, 329142, 329143, 329144, 329145, 329146, 329147, 329148, 329149, 329150, 329151, 329152, 329153, 329154, 329155, 329156, 329157) c6( ) c7(T, dD, hl, aio, ea, if, fd, aj, pl, j, fu, qj, eq, qN, pC, bW, azt, aT, ey, K, bU, yj, cp, aW, Y, jT, nC, ik, kP, asl, cj, nw, de); #c1 (CANX) c2(XP_011532967) c3(calnexin isoform X1) c4( ) c5(329158, 329159, 329160, 329161, 329162, 329163, 329164, 329165, 329166, 329167, 329168, 329169, 329170, 329171, 329172, 329173, 329174, 329175, 329176, 329177, 329178, 329179, 329180, 329181, 329182, 329183, 329184, 329185, 329186, 329187, 329188, 329189, 329190, 329191, 329192, 329193, 329194, 329195, 329196, 329197, 329198, 329199, 329200, 329201, 329202, 329203, 329204, 329205, 329206, 329207, 329208, 329209, 329210) c6( ) c7(T, dD, hl, aio, ea, if, fd, aj, pl, j, fu, qj, eq, qN, pC, bW, azt, aT, ey, K, bU, yj, cp, aW, Y, jT, nC, ik, kP, asl, cj, nw, de); #c1(CAP1) c2(XP_011538812) c3(adenylyl cyclase-associated protein 1 isoform X1) c4(336599, 336600, 336601, 336602, 336603, 336604, 336605) c5(336606, 336607, 336608, 336609, 336610, 336611, 336612, 336613, 336614, 336615, 336616, 336617, 336618, 336619, 336620, 336621, 336622, 336623, 336624, 336625, 336626, 336627, 336628, 336629, 336630, 336631, 336632, 336633, 336634, 336635, 336636, 336637, 336638, 336639, 336640, 336641, 336642, 336643, 336644, 336645, 336646, 336647, 336648, 336649, 336650, 336651, 336652, 336653, 336654, 336655, 336656, 336657, 336658, 336659, 336660, 336661, 336662, 336663, 336664, 336665, 336666, 336667, 336668, 336669, 336670, 336671, 336672, 336673, 336674, 336675, 336676, 336677, 336678, 336679, 336680, 336681, 336682, 336683, 336684, 336685, 336686, 336687, 336688, 336689, 336690, 336691, 336692, 336693, 336694, 336695, 336696) c6(336697, 336698, 336699, 336700, 336701, 336702, 336703, 336704, 336705, 336706, 336707, 336708, 336709, 336710, 336711, 336712, 336713, 336714, 336715, 336716, 336717, 336718, 336719) c7(bL, bM, T, Ke, jx, ci, q, ad, eW, F, afn, aT, h, K, ae, bA, avR, aW, r, tv, aq, tu, avT, cT, rR, avS, CZ, B); #c1(CAP1) c2(XP_011538817) c3(adenylyl cyclase-associated protein 1 isoform X2) c4(336720, 336721, 336722) c5(336723, 336724, 336725, 336726, 336727, 336728, 336729, 336730, 336731, 336732, 336733, 336734, 336735, 336736, 336737, 336738, 336739, 336740, 336741, 336742, 336743, 336744, 336745, 336746, 336747, 336748, 336749, 336750, 336751, 336752, 336753, 336754, 336755, 336756, 336757, 336758, 336759, 336760, 336761, 336762, 336763, 336764) c6(336765, 336766, 336767, 336768, 336769, 336770, 336771, 336772, 336773, 336774, 336775, 336776) c7(bL, bM, T, Ke, jx, ci, q, ad, eW, F, afn, aT, h, K, ae, bA, avR, aW, r, tv, aq, tu, avT, cT, rR, avS, CZ, B); #c1(CAP2) c2(NP_006357) c3(adenylyl cyclase-associated protein 2) c4(164329, 164330) c5(164331, 164332, 164333, 164334, 164335, 164336) c6( ) c7(wR, B, W); #c1(CAP2) c2(XP_011512535) c3 (adenylyl cyclase-associated protein 2 isoform X1) c4( ) c5(303194, 303195, 303196, 303197, 303198, 303199) c6( ) c7(wR, B, W); #c1(CAPG) c2(NP_001243069) c3(macrophage-capping protein isoform 2) c4(89722) c5(89723, 89724, 89725, 89726, 89727, 89728) c6( ) c7(T, eR, kA, oA, aA, n, fr, aY, hF, ai, cd, dY, ec, ql, oj, aT, h, GQ, K, af, Uf, cH, aW, lo, aF, CQ, g, cm, wT, W, eD, Hb, lM, pJ, bt, iu); #c1(CAPG) c2(XP_011531425) c3(macrophage-capping protein isoform X1) c4(327520, 327521, 327522, 327523) c5(327524, 327525, 327526, 327527, 327528, 327529, 327530, 327531, 327532, 327533, 327534, 327535, 327536, 327537, 327538, 327539, 327540, 327541, 327542, 327543, 327544, 327545, 327546, 327547, 327548, 327549, 327550, 327551, 327552, 327553, 327554, 327555, 327556, 327557, 327558, 327559, 327560, 327561, 327562, 327563) c6( ) c7(T, eR, kA, oA, aA, n, fr, aY, hF, ai, cd, dY, ec, ql, oj, aT, h, GQ, K, af, Uf, cH, aW, lo, aF, CQ, g, cm, wT, W, eD, Hb, lM, pJ, bt, iu); #c1(CAPN1) c2(XP_011543594) c3(calpain-1 catalytic subunit isoform X1) c4(342518, 342519, 342520, 342521, 342522, 342523, 342524, 342525, 342526, 342527, 342528) c5(342529, 342530, 342531, 342532, 342533, 342534, 342535, 342536, 342537, 342538, 342539, 342540, 342541, 342542, 342543, 342544, 342545, 342546, 342547, 342548, 342549, 342550, 342551, 342552, 342553, 342554, 342555, 342556, 342557, 342558, 342559, 342560, 342561, 342562, 342563, 342564, 342565, 342566, 342567, 342568, 342569, 342570, 342571, 342572, 342573, 342574, 342575, 342576, 342577, 342578, 342579, 342580, 342581, 342582, 342583, 342584, 342585, 342586, 342587, 342588, 342589, 342590, 342591, 342592, 342593, 342594, 342595, 342596, 342597, 342598, 342599, 342600, 342601, 342602, 342603, 342604, 342605, 342606, 342607, 342608, 342609, 342610, 342611, 342612, 342613, 342614) c6( ) c7(bL, sS, kB, Y, ff, rf, uY, en, af, ad); #c1(CAPN10) c2(NP_075571) c3(calpain-10 isoform a) c4(213986, 213987, 213988) c5(213989, 213990, 213991, 213992, 213993, 213994) c6( ) c7(bL, Gl, aC, dD, iH, eR, aZ, ea, e, aA, cz, n, aj, dG, j, ci, nA, Gn, v, eq, q, J, ad, aex, jy, K, uV, bq, qM, af, aev, kl, cH, azJ, aeS, r, Mz, dq, aF, uC, qa, bJ, W, bE, Fi, mg, vy, mf, ei, rA, ao); #c1(CAPN10) c2(NP_075573) c3(calpain-10 isoform c) c4(213995, 213996) c5(213997, 213998, 213999, 214000, 214001, 214002, 214003, 214004, 214005, 214006, 214007, 214008, 214009, 214010, 214011, 214012, 214013, 214014, 214015, 214016, 214017, 214018, 214019, 214020) c6( ) c7(bL, Gl, aC, dD, iH, eR, aZ, ea, e, aA, cz, n, aj, dG, j, ci, nA, Gn, v, eq, q, J, ad, aex, jy, K, uV, bq, qM, af, aev, kl, cH, azJ, aeS, r, Mz, dq, aF, uC, qa, bJ, W, bE, Fi, mg, vy, mf, ei, rA, ao); #c1(CAPN13) c2(NP_653176) c3(calpain-13) c4(238951, 238952) c5(238953, 238954, 238955, 238956, 238957, 238958, 238959, 238960, 238961, 238962, 238963, 238964) c6( ) c7(cz); #c1(CAPN14) c2(NP_001138594) c3(calpain-14) c4(57134, 57135) c5(57136, 57137, 57138, 57139, 57140, 57141, 57142, 57143, 57144, 57145, 57146, 57147) c6( ) c7(zo, akc); #c1(CAPN2) c2(NP_001139540) c3(calpain-2 catalytic subunit isoform 2) c4(59471) c5(59472, 59473, 59474, 59475, 59476, 59477) c6( ) c7(aW, U, cj, aT, uY, an, bt); #c1(CAPN2) c2(NP_001739) c3(calpain-2 catalytic subunit isoform 1) c4(120653, 120654, 120655) c5(120656, 120657, 120658, 120659, 120660, 120661, 120662, 120663, 120664, 120665, 120666, 120667) c6( ) c7(aW, U, cj, aT, uY, an, bt); #c1(CAPN2) c2(XP_011542588) c3(calpain-2 catalytic subunit isoform X1) c4( ) c5(341154, 341155, 341156, 341157, 341158, 341159) c6( ) c7(aW, U, cj, aT, uY, an, bt); #c1(CAPN3) c2(NP_000061) c3(calpain-3 isoform a) c4(1087) c5(1088, 1089, 1090, 1091, 1092, 1093, 1094, 1095) c6( ) c7(bL, aiu, T, aeu, aj, v, Rk, J, no, azl, Wn, Ae, Pq, oj, Ak, bl, atO, li, VC, aqZ, Y, xs, dr, adu, ac, au, azK, km, cj, azM, oh, rx, Fw, B, jr); #c1(CAPN3) c2(NP_077320) c3(calpain-3 isoform b) c4(215216, 215217, 215218, 215219) c5(215220, 215221, 215222, 215223, 215224, 215225, 215226, 215227, 215228, 215229, 215230, 215231, 215232, 215233, 215234, 215235, 215236, 215237, 215238, 215239, 215240) c6( ) c7(bL, aiu, T, aeu, aj, v, Rk, J, no, azL, Wn, Ae, Pq, oj, Ak, bl, atO, li, VC, aqZ, Y, xs, dr, adu, ac, au, azK, km, cj, azM, oh, rx, Fw, B, jr); #c1(CAPN3) c2(NP_775110) c3(calpain-3 isoform c) c4(249599) c5(249600, 249601, 249602, 249603, 249604, 249605, 249606) c6( ) c7(bL, aiu, T, aeu, aj, v, Rk, J, no, azl, Wn, Ae, Pq, oj, Ak, bl, atO, li, VC, aqZ, Y, xs, dr, adu, ac, au, azk, km, cj, azM, oh, rx, Fw, B, jr); #c1(CAPN3) c2(NP_775111) c3(calpain-3 isoform d) c4(249607, 249608) c5(249609, 249610, 249611, 249612, 249613, 249614, 249615, 249616, 249617, 249618, 249619, 249620, 249621, 249622) c6( ) c7(bL, aiu, T, aeu, aj, v, Rk, J, no, azl, Wn, Ae, Pq, oj, Ak, bl, atO, li, VC, aqZ, Y, xs, dr, adu, ac, au, azk, km, cj, azM, oh, rx, Fw, B, jr); #c1(CAPN3) c2(NP_775112) c3(calpain-3 isoform e) c4( ) c5(249623, 249624, 249625, 249626, 249627, 249628, 249629, 249630, 249631) c6( ) c7(bL, aiu, T, aeu, aj, v, Rk, J, no, azl, Wn, Ae, Pq, oj, Ak, bl, atO, li, VC, aqZ, Y, xs, dr, adu, ac, au, azK, km, cj, azM, oh, rx, Fw, B, jr); #c1 (CAPN5) c2(NP_004046) c3(calpain-5) c4(143137) c5(143138, 143139, 143140, 143141, 143142) c6( ) c7(bL, T, dD, aZ, e, cz, v, eq, qW, av, Pq, BJ, K, Ha, cY, kl, cl, t, bz, nB, hL, m, oQ); #c1(CAPN5) c2(XP_011543527) c3(calpain-5 isoform X1) c4(342421, 342422, 342423) c5(342424, 342425, 342426, 342427, 342428, 342429, 342430, 342431, 342432, 342433, 342434, 342435, 342436, 342437, 342438, 342439, 342440, 342441, 342442, 342443, 342444, 342445, 342446) c6( ) c7(bL, T, dD, aZ, e, cz, v, eq, qW, av, Pq, BJ, K, Ha, cY, kl, cl, t, bz, nB, hL, m, oQ); #c1 (CAPN6) c2(NP_055104) c3(calpain-6) c4(178963, 178964) c5(178965, 178966, 178967, 178968, 178969, 178970, 178971, 178972, 178973, 178974, 178975, 178976) c6( ) c7(bK); #c1(CAPN7) c2(NP_055111) c3(calpain-7) c4(179005) c5(179006, 179007, 179008, 179009, 179010, 179011) c6( ) c7(W); #c1(CAPN9) c2(NP_006606) c3(calpain-9 isoform 1) c4(166581) c5(166582, 166583, 166584, 166585, 166586, 166587) c6( ) c7(cl, aF, aA, hL, cj); #c1(CAPN9) c2(NP_057536) c3(calpain-9 isoform 2) c4(191293) c5(191294, 191295, 191296, 191297, 191298, 191299) c6( ) c7(cl, aF, aA, hL, cj); #c1(CAPN9) c2(XP_005273067) c3(calpain-9 isoform X4) c4(284285) c5(284286, 284287, 284288, 284289, 284290, 284291) c6( ) c7(cl, aF, aA, hl, cj); #c1(CAPN9) c2(XP_011542319) c3(calpain-9 isoform X1) c4( ) c5(340912) c6( ) c7(cl, aF, aA, hL, cj); #c1(CAPNS1) c2(NP_001289561) c3(calpain small subunit 1 isoform 1) c4(114274) c5(114275, 114276, 114277, 114278, 114279, 114280) c6( ) c7(Mb, T, d, Dh, sw, aW, aT, n, aq, v, eA, B, ad); #c1(CAPRIN1) c2(NP_005889) c3(caprin-1 isoform 1) c4(160137) c5( ) c6( ) c7(ey, K); #c1(CAPRIN1) c2(NP_976240) c3(caprin-1 isoform 2) c4(263041) c5(263042, 263043, 263044, 263045, 263046, 263047, 263048, 263049, 263050, 263051, 263052, 263053, 263054, 263055, 263056, 263057) c6( ) c7(ey, K); #c1(CAPRIN2) c2(NP_001002259) c3(caprin-2 isoform 1) c4(19470) c5(19471, 19472, 19473, 19474, 19475, 19476) c6( ) c7(iO, j, EQ); #c1(CAPRIN2) c2(NP_001193785) c3(caprin-2 isoform 4) c4(82613) c5(82614, 82615, 82616, 82617, 82618, 82619) c6( ) c7(iO, j, EQ); #c1 (CAPRIN2) c2(NP_076414) c3(caprin-2 isoform 2) c4(214091) c5(214092, 214093, 214094, 214095, 214096, 214097) c6( ) c7(iO, j, EQ); #c1(CAPRIN2) c2(NP_115532) c3(caprin-2 isoform 3) c4(223108) c5(223109, 223110, 223111, 223112, 223113, 223114) c6( ) c7(iO, j, EQ); #c1(CAPRIN2) c2(XP_006719200) c3(caprin-2 isoform X1) c4(292298) c5(292299, 292300, 292301, 292302, 292303, 292304) c6( ) c7(iO, j, EQ); #c1(CAPRIN2) c2(XP_006719202) c3(caprin-2 isoform X3) c4( ) c5(292305, 292306, 292307, 292308, 292309, 292310) c6( ) c7(iO, j, EQ); #c1(CAPRIN2) c2(XP_011519131) c3(caprin-2 isoform X15) c4(313732) c5( ) c6( ) c7(iO, j, EQ); #c1(CAPS) c2(NP_004049) c3(calcyphosin isoform a) c4(143150, 143151) c5(143152, 143153, 143154, 143155, 143156, 143157, 143158, 143159, 143160, 143161, 143162, 143163) c6( ) c7(hH, ae, ih, ad, awP); #c1(CAPS) c2(NP_542157) c3(calcyphosin isoform b) c4(231445) c5(231446, 231447, 231448, 231449, 231450, 231451) c6( ) c7(hH, ae, ih, ad, awP); #c1(CAPS2) c2(NP_001273476) c3(calcyphosin-2 isoform 2) c4(104975) c5( ) c6( ) c7(rz, bN); #c1(CAPS2) c2(NP_001273478) c3(calcyphosin-2 isoform 4) c4( ) c5(104976, 104977, 104978, 104979, 104980) c6( ) c7(rz, bN); #c1(CAPS2) c2(NP_115995) c3(calcyphosin-2 isoform 1) c4(225154) c5( ) c6( ) c7(rz, bN); #c1(CAPS2) c2(XP_005269251) c3(calcyphosin-2 isoform X12) c4( ) c5(282501, 282502, 282503, 282504, 282505, 282506) c6( ) c7(rz, bN); #c1(CAPS2) c2(XP_006719711) c3(calcyphosin-2 isoform X2) c4( ) c5(292904, 292905, 292906, 292907, 292908, 292909) c6( ) c7(rz, bN); #c1(CAPS2) c2(XP_006719712) c3(calcyphosin-2 isoform X5) c4(292910) c5( ) c6( ) c7(rz, bN); #c1(CAPS2) c2(XP_006719713) c3(calcyphosin-2 isoform X11) c4( ) c5(292911) c6( ) c7(rz, bN); #c1(CAPS2) c2(XP_011537190) c3(calcyphosin-2 isoform X8) c4(334953) c5( ) c6( ) c7(rz, bN); #c1(CAPS2) c2(XP_011537193) c3(calcyphosin-2 isoform X3) c4( ) c5(334954) c6( ) c7(rz, bN); #c1(CAPS2) c2(XP_011537194) c3(calcyphosin-2 isoform X1) c4( ) c5(334955, 334956, 334957, 334958, 334959) c6( ) c7(rz, bN); #c1(CAPSL) c2(NP_001036090) c3(calcyphosin-like protein) c4( ) c5(33963, 33964, 33965, 33966, 33967, 33968, 33969, 33970, 33971, 33972, 33973, 33974, 33975) c6( ) c7(A, bc); #c1(CAPSL) c2(XP_006714507) c3(calcyphosin-like protein isoform X1) c4(287876, 287877) c5(287878, 287879, 287880) c6( ) c7(A, bc); #c1(CAPZA2) c2(NP_006127) c3(F-actin-capping protein subunit alpha-2) c4(162260, 162261) c5(162262, 162263, 162264, 162265, 162266, 162267, 162268, 162269, 162270, 162271, 162272, 162273) c6( ) c7(ey); #c1(CAPZA3) c2(NP_201585) c3(F-actin-capping protein subunit alpha-3) c4(228298) c5(228299, 228300, 228301, 228302, 228303, 228304) c6( ) c7(cd, EQ); #c1(CARDIO) c2(NP_055365) c3(caspase recruitment domain-containing protein 10) c4(180612, 180613, 180614) c5(180615, 180616, 180617, 180618, 180619, 180620, 180621, 180622, 180623, 180624, 180625, 180626, 180627, 180628, 180629, 180630, 180631, 180632, 180633, 180634, 180635, 180636) c6( ) c7(bM, T, eM, dH, aW, aT, j, bh, fr, t, bz); #c1(CARD11) c2(NP_115791) c3(caspase recruitment domain-containing protein 11) c4(223910, 223911) c5(223912, 223913, 223914, 223915, 223916, 223917) c6( ) c7(aox, Cv, T, fM, azN, oe, eE, jh, fr, C, fD, ij, Wt); #c1(CARD11) c2(XP_011513889) c3(caspase recruitment domain-containing protein II isoform X2) c4( ) c5(306310, 306311, 306312, 306313, 306314, 306315) c6( ) c7(aox, Cv, T, fM, azN, oe, eE, jh, fr, C, fD, ij, Wt); #c1(CARD14) c2(NP_001244899) c3(caspase recruitment domain-containing protein 14 isoform 3) c4(92315, 92316) c5(92317, 92318, 92319, 92320, 92321, 92322) c6( ) c7(ka, dt, kh, axA, jX, V, wD, hX, jY, hb, ds, aT, hD, azR, aW, azP, cr, azO, eE, kk, dS, jV, ac, ki, azl, kq, jQ); #c1(CARD14) c2(NP_077015) c3(caspase recruitment domain-containing protein 14 isoform 1) c4(214741) c5(214742, 214743, 214744, 214745, 214746, 214747, 214748, 214749, 214750, 214751, 214752) c6( ) c7(ka, dt, kh, axA, jX, V, wD, hX, jY, hb, ds, aT, hD, azR, aW, azP, cr, azO, eE, kk, dS, jV, ac, ki, azQ, kq, jQ); #c1(CARD14) c2(NP_438170) c3(caspase recruitment domain-containing protein 14 isoform 2) c4(229118) c5(229119, 229120, 229121, 229122, 229123, 229124, 229125, 229126, 229127, 229128, 229129) c6( ) c7(ka, dt, kh, axA, jX, V, wD, hX, jY, hb, ds, aT, hD, azR, aW, azP, cr, azO, eE, kk, dS, jV, ac, ki, azl, kq, jQ); #c1(CARD16) c2(NP_001017534) c3(caspase recruitment domain-containing protein 16 isoform 1) c4(26586) c5(26587, 26588, 26589, 26590, 26591, 26592, 26593, 26594) c6( ) c7(T, rR, aE, c, qt, aW, jx, aT, aA, n, oY, aF, K, g, ir, bt, B); #c1(CARD16) c2(NP_443121) c3(caspase recruitment domain-containing protein 16 isoform 2) c4(229540) c5(229541, 229542, 229543, 229544, 229545, 229546, 229547, 229548, 229549, 229550, 229551, 229552, 229553) c6( ) c7(T, rR, aE, c, qt, aW, jx, aT, aA, n, oY, aF, K, g, ir, bt, B); #c1(CARD6) c2(NP_115976) c3(caspase recruitment domain-containing protein 6) c4(225028, 225029, 225030) c5(225031, 225032, 225033, 225034, 225035, 225036, 225037, 225038, 225039, 225040, 225041, 225042, 225043, 225044, 225045, 225046, 225047, 225048, 225049) c6( ) c7(cd); #c1(CARD8) c2(NP_001171829) c3(caspase recruitment domain-containing protein 8 isoform a) c4(71923, 71924) c5(71925, 71926, 71927, 71928, 71929, 71930) c6( ) c7(oO, T, dD, ea, aw, cz, bB, cl, eq, ai, o, M, aT, af, NX, azl, mR, azS, bf, aW, r, fe, iV, ac, bE, dc, azT, bm, cj, y); #c1(CARD8) c2(NP_001171830) c3(caspase recruitment domain-containing protein 8 isoform b) c4(71931) c5(71932, 71933, 71934, 71935, 71936, 71937) c6( ) c7(oO, T, dD, ea, aw, cz, bB, cl, eq, ai, o, M, aT, af, NX, azU, mR, azS, bf, aW, r, fe, iV, ac, bE, dc, azT, bm, cj, y); #c1(CARD8) c2(NP_001171833) c3(caspase recruitment domain-containing protein 8 isoform d) c4( ) c5(71938, 71939, 71940, 71941, 71942, 71943) c6( ) c7(oO, T, dD, ea, aw, cz, bB, cl, eq, ai, o, M, aT, af, NX, azU, mR, azS, bf, aW, r, fe, iV, ac, bE, dc, azT, bm, cj, y); #c1 (CARD8) c2(XP_006723167) c3(caspase recruitment domain-containing protein 8 isoform X10) c4(295015) c5(295016, 295017, 295018, 295019, 295020) c6( ) c7(oO, T, dD, ea, aw, cz, bB, cL, eq, ai, o, M, aT, af, NX, azU, mR, azS, bf, aW, r, fe, iV, ac, bE, dc, azT, bm, cj, y); #c1(CARD8) c2(XP_011524949) c3(caspase recruitment domain-containing protein 8 isoform X6) c4( ) c5(319432) c6( ) c7(oO, T, dD, ea, aw, cz, bB, cl, eq, ai, o, M, aT, af, NX, azU, mR, azS, bf, aW, r, fe, iV, ac, bE, dc, azT, bm, cj, y); #c1(CARD8) c2(XP_011524950) c3(caspase recruitment domain-containing protein 8 isoform X7) c4( ) c5(319433) c6( ) c7(oO, T, dD, ea, aw, cz, bB, cl, eq, ai, o, M, aT, af, NX, azl, mR, azS, bf, aW, r, fe, iV, ac, bE, dc, azT, bm, cj, y); #c1(CARD8) c2(XP_011524953) c3(caspase recruitment domain-containing protein 8 isoform X12) c4(319434) c5(319435, 319436, 319437, 319438, 319439) c6( ) c7(oO, T, dD, ea, aw, cz, bB, cL, eq, ai, o, M, aT, af, NX, azl, mR, azS, bf, aW, r, fe, iV, ac, bE, dc, azT, bm, cj, y); #c1(CARDS) c2(NP_434700) c3(caspase recruitment domain-containing protein 9 isoform 1) c4(229084, 229085) c5(229086, 229087, 229088, 229089, 229090, 229091) c6( ) c7(azV, fM, azW, azX, Pa, py, mR, hl, fe, n, wR, CZ, ey, iV); #c1(CARD9) c2(NP_434701) c3(caspase recruitment domain-containing protein 9 isoform 2) c4(229092) c5(229093, 229094, 229095, 229096, 229097, 229098, 229099, 229100, 229101, 229102, 229103, 229104) c6( ) c7(azV, fM, azW, azX, Pa, py, mR, hl, fe, n, wR, CZ, ey, iV); #c1(CARF) c2(NP_001098056) c3(calcium-responsive transcription factor isoform 1) c4(41315, 41316) c5(41317, 41318, 41319, 41320, 41321, 41322, 41323, 41324, 41325, 41326, 41327, 41328, 41329, 41330, 41331, 41332, 41333, 41334, 41335) c6( ) c7(T, K, nA); #c1(CARF) c2(NP_001269839) c3(calcium-responsive transcription factor isoform 2) c4(102927) c5(102928, 102929, 102930, 102931, 102932, 102933) c6( ) c7(T, K, nA); #c1 (CARF) c2(NP_001269841) c3(calcium-responsive transcription factor isoform 3) c4(102934) c5(102935, 102936, 102937, 102938, 102939, 102940) c6( ) c7(T, K, nA); #c1(CARF) c2(XP_005246920) c3(calcium-responsive transcription factor isoform X4) c4(267494) c5( ) c6( ) c7(T, K, nA); #c1(CARF) c2(XP_006712822) c3(calcium-responsive transcription factor isoform X5) c4(286844, 286845, 286846) c5( ) c6( ) c7(T, K, nA); #c1(CARF) c2(XP_006712823) c3(calcium-responsive transcription factor isoform X7) c4(286847) c5(286848, 286849, 286850, 286851, 286852, 286853, 286854, 286855, 286856, 286857, 286858, 286859, 286860, 286861, 286862, 286863, 286864, 286865, 286866, 286867, 286868, 286869, 286870, 286871, 286872, 286873, 286874, 286875, 286876, 286877, 286878, 286879, 286880, 286881) c6( ) c7(T, K, nA); #c1(CARKD) c2(NP_001229810) c3(ATP-dependent (S)-NAD(P)H-hydrate dehydratase isoform b) c4(85293) c5( ) c6( ) c7(aA); #c1(CARKD) c2(NP_001229811) c3(ATP-dependent (S)-NAD (P)H-hydrate dehydratase isoform c) c4(85294, 85295) c5(85296, 85297, 85298, 85299, 85300, 85301, 85302, 85303, 85304, 85305, 85306, 85307) c6( ) c7(aA); #c1(CARKD) c2(NP_001229812) c3(ATP-dependent (S)-NAD(P)H-hydrate dehydratase isoform d) c4(85308) c5(85309, 85310, 85311, 85312, 85313, 85314, 85315, 85316, 85317, 85318, 85319, 85320) c6( ) c7(aA); #c1(CARKD) c2(NP_060680) c3(ATP-dependent (S)-NAD(P)H-hydrate dehydratase isoform a) c4(197137) c5(197138, 197139, 197140, 197141, 197142, 197143) c6( ) c7(aA); #c1(CARMI) c2(NP_954592) c3(histone-arginine methyltransferase CARMI) c4(261079) c5(261080, 261081, 261082, 261083, 261084, 261085) c6( ) c7(ae, T, bM, B, bv, eM, lp, aW, Y, vy, aT, kf, r, cr, ci, bz, q, ad); #c1(CARMI) c2(XP_005259765) c3(histone-arginine methyltransferase CARMI isoform X1) c4(276291) c5(276292, 276293, 276294, 276295, 276296, 276297) c6( ) c7(ae, T, bM, B, bv, eM, lp, aW, Y, vy, aT, kf, r, cr, ci, bz, q, ad); #c1(CARMI) c2(XP_011525939) c3(histone-arginine methyltransferase CARMI isoform X2) c4( ) c5(320348) c6( ) c7(ae, T, bM, B, bv, eM, lp, aW, Y, vy, aT, kf, r, cr, ci, bz, q, ad); #c1(CARS) c2(NP_001014437) c3(cysteine-tRNA ligase, cytoplasmic isoform c) c4(25903) c5(25904, 25905, 25906, 25907, 25908, 25909) c6( ) c7(bL, T, A, fM, ahc, VT, aj, dy, m, C, eA, B, J, bN); #c1(CARS) c2(NP_001742) c3(cysteine-tRNA ligase, cytoplasmic isoform b) c4(120668) c5(120669, 120670, 120671, 120672, 120673, 120674) c6( ) c7(bL, T, A, fM, ahc, VT, aj, dy, m, C, eA, B, J, bN); #c1(CARS) c2(NP_644802) c3(cysteine-tRNA ligase, cytoplasmic isoform a) c4(238535, 238536) c5(238537, 238538, 238539, 238540, 238541, 238542, 238543, 238544, 238545, 238546, 238547, 238548) c6( ) c7(bL, T, A, fM, ahc, VT, aj, dy, m, C, eA, B, J, bN); #c1(CARS) c2(XP_006718404) c3(cysteine-tRNA ligase, cytoplasmic isoform X1) c4(291824) c5(291825, 291826, 291827, 291828, 291829, 291830) c6( ) c7(bL, T, A, fM, ahc, VT, aj, dy, m, C, eA, B, J, bN); #c1(CARTPT) c2(NP_004282) c3(cocaine- and amphetamine-regulated transcript protein preproprotein) c4(144816) c5(144817, 144818, 144819, 144820, 144821, 144822, 144823, 144824) c6(144825, 144826) c7(bL, T, dD, ea, oe, cz, Gd, bS, v, ct, eq, cd, azY, PW, pN, ql, Fo, pX, XY, aql, M, dV, io, cy, bK, he, Yi, Qe, gl, cu, Z, FT, hq, ek, kT, ok, ac, wl, awl, hr, aqm, st); #c1(CASC1) c2(NP_001076441) c3 (protein CASC1 isoform c) c4(37432, 37433) c5(37434, 37435, 37436, 37437, 37438, 37439, 37440, 37441, 37442, 37443, 37444, 37445, 37446, 37447, 37448, 37449, 37450, 37451) c6( ) c7(av, auM, bz, iu, g); #c1(CASC1) c2(NP_060742) c3(protein CASC1 isoform a) c4(197588) c5( ) c6( ) c7(av, auM, bz, iu, g); #c1(CASC1) c2(XP_005253466) c3(protein CASC1 isoform X5) c4(272517) c5( ) c6( ) c7(av, auM, bz, iu, g); #c1(CASC1) c2(XP_006719165) c3(protein CASC1 isoform X8) c4( ) c5(292238, 292239, 292240, 292241, 292242, 292243) c6( ) c7(av, auM, bz, iu, g); #c1(CASC1) c2(XP_011519025) c3(protein CASC1 isoform X2) c4(313466, 313467, 313468) c5(313469, 313470, 313471, 313472, 313473, 313474, 313475) c6( ) c7(av, auM, bz, iu, g); #c1(CASC1) c2(XP_011519026) c3 (protein CASC1 isoform X3) c4(313476) c5( ) c6( ) c7(av, auM, bz, iu, g); #c1(CASC1) c2(XP_011519027) c3(protein CASC1 isoform X1) c4( ) c5(313477, 313478, 313479, 313480, 313481, 313482, 313483, 313484, 313485, 313486, 313487, 313488, 313489, 313490, 313491, 313492, 313493, 313494) c6( ) c7(av, auM, bz, iu, g); #c1(CASC1) c2(XP_011519028) c3(protein CASC1 isoform X6) c4(313495) c5(313496, 313497, 313498, 313499, 313500) c6( ) c7(av, auM, bz, iu, g); #c1(CASC3) c2(XP_005257220) c3(protein CASC3 isoform X1) c4(274787, 274788) c5(274789, 274790, 274791, 274792, 274793, 274794, 274795, 274796, 274797, 274798, 274799, 274800, 274801, 274802, 274803, 274804, 274805) c6( ) c7(aW, T, K, y, aT); #c1(CASC4) c2(NP_612432) c3(protein CASC4 isoform a) c4(236468, 236469, 236470, 236471, 236472) c5(236473, 236474, 236475, 236476, 236477, 236478, 236479, 236480, 236481, 236482, 236483, 236484, 236485, 236486, 236487, 236488, 236489, 236490, 236491, 236492, 236493, 236494, 236495, 236496) c6( ) c7(ac); #c1(CASC4) c2(NP_816929) c3(protein CASC4 isoform b) c4(253040) c5(253041, 253042, 253043, 253044, 253045, 253046, 253047) c6( ) c7(ac); #c1(CASC4) c2(XP_011519472) c3(protein CASC4 isoform X1) c4( ) c5(314139, 314140, 314141, 314142, 314143) c6( ) c7(ac); #c1(CASC5) c2(NP_653091) c3(protein CASC5 isoform 2) c4(238872) c5(238873, 238874, 238875, 238876, 238877, 238878) c6( ) c7(x, av, T, aii, Y, azZ, k, bX, bu, bt, aij); #c1(CASC5) c2(NP_733468) c3(protein CASC5 isoform 1) c4(246895) c5(246896, 246897, 246898, 246899, 246900, 246901) c6( ) c7(x, av, T, aii, Y, azZ, k, bX, bu, bt, aij); #c1(CASC5) c2(XP_011520119) c3(protein CASC5 isoform X2) c4(315010) c5(315011, 315012, 315013, 315014, 315015, 315016) c6( ) c7(x, av, T, aii, Y, azZ, k, bX, bu, bt, aij); #c1(CASD1) c2(NP_075051) c3(CAS1 domain-containing protein 1 precursor) c4(213585) c5(213586, 213587, 213588, 213589, 213590, 213591) c6( ) c7(aW, aAa, NT, aT); #c1(CASK) c2(NP_001119526) c3(peripheral plasma membrane protein CASK isoform 2) c4(44077, 44078) c5(44079, 44080, 44081, 44082, 44083, 44084, 44085, 44086, 44087, 44088, 44089, 44090) c6( ) c7(UR, ci, nA, zP, q, Na, aAc, aAd, hb, NG, hu, aAe, Yl, Ng, zQ, r, nf, aF, aha, OK, KE, bE, FB, aAb); #c1(CASK) c2(NP_001119527) c3(peripheral plasma membrane protein CASK isoform 3) c4(44091, 44092) c5(44093, 44094, 44095, 44096, 44097, 44098, 44099, 44100, 44101, 44102, 44103, 44104) c6( ) c7(UR, ci, nA, zP, q, Na, aAc, aAd, hb, NG, hu, aAe, YI, Ng, zQ, r, nf, aF, aha, QK, KE, bE, FB, aAb); #c1(CASK) c2(NP_003679) c3(peripheral plasma membrane protein CASK isoform 1) c4(140133, 140134, 140135, 140136, 140137) c5(140138, 140139, 140140, 140141, 140142, 140143, 140144, 140145, 140146, 140147, 140148, 140149, 140150, 140151, 140152, 140153, 140154, 140155, 140156, 140157, 140158, 140159, 140160, 140161, 140162, 140163, 140164, 140165, 140166, 140167) c6( ) c7(UR, ci, nA, zP, q, Na, aAc, aAd, hb, NG, hu, aAe, YI, Ng, zQ, r, nf, aF, aha, QK, KE, bE, FB, aAb); #c1(CASK) c2(XP_011542296) c3 (peripheral plasma membrane protein CASK isoform X3) c4(340888) c5(340889, 340890, 340891, 340892, 340893, 340894) c6( ) c7(UR, ci, nA, zP, q, Na, aAc, aAd, hb, NG, hu, aAe, YI, Ng, zQ, r, nf, aF, aha, QK, KE, bE, FB, aAb); #c1(CASP1) c2(NP_001244048) c3(caspase-l isoform beta precursor) c4(91844) c5(91845, 91846, 91847, 91848, 91849, 91850, 91851, 91852, 91853, 91854, 91855, 91856, 91857, 91858, 91859) c6( ) c7(oO, bM, T, fR, ea, al, ci, UI, xE, aE, Bc, dV, Ds, K, zy, akX, aAg, aW, Ls, mQ, ih, CT, aoD, g, eE, cm, iA, wE, mx, U, cj, hh, ajy, dD, aw, fr, ct, PJ, zP, cd, eW, aAh, h, lG, fe, Ca, ajz, Zb, hq, ar, Ei, E, eo, cB, Dw, aAf, fG, aA, azF, v, j, C, V, eA, aT, cy, bq, ae, af, aAi, le, bf, s, iU, Y, lo, bu, pb, c, AH, Uq, wR, Da, dy, fD, bt, hS, kV, qX, ZI, iW, bS, eq, ad, sO, NY, ds, abq, jo, fb, NM, sj, d, BO, yx, nh, eZ, dq, aF, Fq, iV, Z, bz, bF, es, gk, DT, bE, y); #c1(CASP1) c2(NP_150634) c3(caspase-l isoform alpha precursor) c4(228157, 228158, 228159) c5(228160, 228161, 228162, 228163, 228164, 228165, 228166, 228167, 228168, 228169, 228170, 228171, 228172, 228173, 228174) c6( ) c7(oO, bM, T, fR, ea, al, ci, Ul, xE, aE, Bc, dV, Ds, K, zy, akX, aAg, aW, Ls, mQ, ih, CT, aoD, g, eE, cm, iA, wE, mx, U, cj, hh, ajy, dD, aw, fr, ct, PJ, zP, cd, eW, aAh, h, lG, fe, Ca, ajz, Zb, hq, ar, Ei, E, eo, cB, Dw, aAf, fG, aA, azf, v, j, C, V, eA, aT, cy, bq, ae, af, aAi, le, bf, s, iU, Y, lo, bu, pb, c, AH, Uq, wR, Da, dy, fD, bt, hS, kV, qX, ZI, iW, bS, eq, ad, sO, NY, ds, abq, jo, fb, NM, sj, d, BO, yx, nh, eZ, dq, aF, Fq, iV, Z, bz, bF, es, gk, DT, bE, y); #c1 (CASP1) c2(NP_150635) c3(caspase-1 isoform gamma) c4(228175) c5(228176, 228177, 228178, 228179, 228180, 228181) c6( ) c7(oO, bM, T, fR, ea, al, ci, Ul, xE, aE, Bc, dV, Ds, K, zy, akX, aAg, aW, Ls, mQ, ih, CT, aoD, g, eE, cm, iA, wE, mx, U, cj, hh, ajy, dD, aw, fr, ct, PJ, zP, cd, eW, aAh, h, IG, fe, Ca, ajz, Zb, hq, ar, Ei, E, eo, cB, Dw, aAf, fG, aA, azF, v, j, C, V, eA, aT, cy, bq, ae, af, aAi, le, bf, s, iU, Y, lo, bu, pb, c, AH, Uq, wR, Da, dy, fD, bt, hS, kV, qX, ZI, iW, bS, eq, ad, sO, NY, ds, abq, jo, fb, NM, sj, d, BO, yx, nh, eZ, dq, aF, Fq, iV, Z, bz, bF, es, gk, DT, bE, y); #c1(CASP1) c2(NP_150636) c3(caspase-1 isoform delta) c4(228182) c5(228183, 228184, 228185, 228186, 228187, 228188, 228189, 228190, 228191, 228192, 228193, 228194, 228195) c6( ) c7(oO, bM, T, fR, ea, al, ci, UI, xE, aE, Bc, dV, Ds, K, zy, akX, aAg, aW, Ls, mQ, ih, CT, aoD, g, eE, cm, iA, wE, mx, U, cj, hh, ajy, dD, aw, fr, ct, PJ, zP, cd, eW, aAh, h, lG, fe, Ca, ajz, Zb, hq, ar, Ei, E, eo, cB, Dw, aAf, fG, aA, azF, v, j, C, V, eA, aT, cy, bq, ae, af, aAi, le, bf, s, iU, Y, lo, bu, pb, c, AH, Uq, wR, Da, dy, fD, bt, hS, kV, qX, ZI, iW, bS, eq, ad, sO, NY, ds, abq, jo, fb, NM, sj, d, BO, yx, nh, eZ, dq, aF, Fq, iV, Z, bz, bF, es, gk, DT, bE, y); #c1(CASP1) c2(NP_150637) c3(caspase-1 isoform epsilon) c4(228196) c5(228197, 228198, 228199, 228200, 228201, 228202, 228203, 228204, 228205, 228206, 228207, 228208, 228209) c6( ) c7(oO, bM, T, fR, ea, al, ci, Ul, xE, aE, Bc, dV, Ds, K, zy, akX, aAg, aW, Ls, mQ, ih, CT, aoD, g, eE, cm, iA, wE, mx, U, cj, hh, ajy, dD, aw, fr, ct, PJ, zP, cd, eW, aAh, h, lG, fe, Ca, ajz, Zb, hq, ar, Ei, E, eo, cB, Dw, aAf, fG, aA, azF, v, j, C, V, eA, aT, cy, bq, ae, af, aAi, le, bf, s, iU, Y, lo, bu, pb, c, AH, Uq, wR, Da, dy, fD, bt, hS, kV, qX, ZI, iW, bS, eq, ad, sO, NY, ds, abq, jo, fb, NM, sj, d, BO, yx, nh, eZ, dq, aF, Fq, iV, Z, bz, bF, es, gk, DT, bE, y); #c1 (CASP1) c2(XP_011541319) c3(caspase-1 isoform X1) c4( ) c5(339917) c6( ) c7(oO, bM, T, FR, ea, al, ci, UI, xE, aE, Bc, dV, Ds, K, zy, akX, aAg, aW, Ls, ml, ih, CT, aoD, g, eE, cm, iA, wE, mx, U, cj, hh, ajy, dD, aw, fr, ct, PJ, zP, cd, eW, aAh, h, lG, fe, Ca, ajz, Zb, hq, ar, Ei, E, eo, cB, Dw, aAf, fG, aA, azF, v, j, C, V, eA, aT, cy, bq, ae, af, aAi, le, bf, s, iU, Y, lo, bu, pb, c, AH, Uq, wR, Da, dy, fD, bt, hS, kV, qX, ZI, iW, bS, eq, ad, sO, NY, ds, abq, jo, fb, NM, sj, d, BO, yx, nh, eZ, dq, aF, Fq, iV, Z, bz, bF, es, gk, DT, bE, y); #c1(CASP10) c2(NP_001193453) c3(caspase-10 isoform 6 preproprotein) c4(82187) c5(82188, 82189, 82190, 82191, 82192, 82193) c6( ) c7(bM, T, iW, ht, NT, aAk, aA, bB, eX, fd, ci, qN, hF, V, ad, x, XW, av, aT, h, eV, hn, dL, j, hu, fb, nJ, ae, ZH, s, CN, iU, aW, sp, Y, eZ, r, aF, g, t, cm, mK, aAj, ck, Dy, c, eM, bE, hk, cj, at, k, m, B); #c1(CASP10) c2(NP_001193471) c3(caspase-10 isoform 5 preproprotein) c4(82217) c5(82218, 82219, 82220, 82221, 82222, 82223) c6( ) c7(bM, T, iW, ht, NT, aAk, aA, bB, eX, fd, ci, qN, hF, V, ad, x, XW, av, aT, h, eV, hn, dL, j, hu, fb, nJ, ae, ZH, s, CN, iU, aW, sp, Y, eZ, r, aF, g, t, cm, mK, aAj, ck, Dy, c, eM, bE, hk, cj, at, k, m, B); #c1 (CASP10) c2(NP_001221) c3(caspase-10 isoform 3 preproprotein) c4(83935) c5(83936, 83937, 83938, 83939, 83940, 83941) c6( ) c7(bM, T, iW, ht, NT, aAk, aA, bB, eX, fd, ci, qN, hF, V, ad, x, XW, av, aT, h, eV, hn, dl, j, hu, fb, nJ, ae, ZH, s, CN, iU, aW, sp, Y, eZ, r, aF, g, t, cm, mK, aAj, ck, Dy, c, eM, bE, hk, cj, at, k, m, B); #c1 (CASP10) c2(NP_116756) c3(caspase-10 isoform 2 preproprotein) c4(226397) c5(226398, 226399, 226400, 226401, 226402, 226403) c6( ) c7(bM, T, iW, ht, NT, aAk, aA, bB, eX, fd, ci, qN, hF, V, ad, x, XW, av, aT, h, eV, hn, dL, j, hu, fb, nJ, ae, ZH, s, CN, iU, aW, sp, Y, eZ, r, aF, g, t, cm, mK, aAj, ck, Dy, c, eM, bE, hk, cj, at, k, m, B); #c1(CASP10) c2(NP_116758) c3(caspase-10 isoform 4) c4(226418) c5(226419, 226420, 226421, 226422, 226423) c6( ) c7(bM, T, iW, ht, NT, aAk, aA, bB, eX, fd, ci, qN, hF, V, ad, x, XW, av, aT, h, eV, hn, dL, j, hu, fb, nJ, ae, ZH, s, CN, iU, aW, sp, Y, eZ, r, aF, g, t, cm, mK, aAj, ck, Dy, c, eM, bE, hk, cj, at, k, m, B); #c1(CASP10) c2(NP_116759) c3(caspase-10 isoform 1 preproprotein) c4(226424) c5(226425, 226426, 226427, 226428, 226429, 226430, 226431) c6( ) c7(bM, T, iW, ht, NT, aAk, aA, bB, eX, fd, ci, qN, hF, V, ad, x, XW, av, aT, h, eV, hn, dL, j, hu, fb, nJ, ae, ZH, s, CN, iU, aW, sp, Y, eZ, r, aF, g, t, cm, mK, aAj, ck, Dy, c, eM, bE, hk, cj, at, k, m, B); #c1(CASP14) c2(NP_036246) c3(caspase-14 precursor) c4(172616, 172617) c5(172618, 172619, 172620, 172621, 172622, 172623, 172624, 172625, 172626, 172627, 172628, 172629, 172630, 172631, 172632, 172633) c6( ) c7(bM, T, dD, dA, Zz, ea, fG, aaQ, yG, zs, if, aA, bB, iW, aj, eq, ci, qN, vY, eh, q, J, cd, ad, ai, eA, av, ft, lg, ds, BJ, hh, qh, aT, h, ji, K, jj, fb, qE, asp, bf, s, oV, xs, r, yc, fe, kp, La, g, as, t, bN, hq, sS, jV, bP, hk, dh, AH, U, gZ, hR, agf, MW, rK, m, aAl, oQ, aS, dy, fD, B); #c1(CASP2) c2(NP_001215) c3(caspase-2 isoform 2) c4(83784) c5(83785, 83786, 83787, 83788, 83789) c6( ) c7(gh, T, iW, jh, aA, bB, hy, fr, nA, V, q, ad, x, aE, aT, K, fb, ae, eL, B, BN, BO, aW, Y, r, g, ac, bE, cj, m, k, bt, cB, ne); #c1(CASP2) c2(NP_116764) c3(caspase-2 isoform 1 preproprotein) c4(226447) c5(226448, 226449, 226450, 226451, 226452, 226453, 226454, 226455) c6( ) c7(gh, T, iW, jh, aA, bB, hy, fr, nA, V, q, ad, x, aE, aT, K, fb, ae, eL, B, BN, BO, aW, Y, r, g, ac, bE, cj, m, k, bt, cB, ne); #c1(CASP2) c2(NP_116765) c3(caspase-2 isoform 3) c4(226456) c5(226457, 226458, 226459, 226460, 226461) c6( ) c7(gh, T, iW, jh, aA, bB, hy, fr, nA, V, q, ad, x, aE, aT, K, fb, ae, eL, B, BN, BO, aW, Y, r, g, ac, bE, cj, m, k, bt, cB, ne); #c1(CASP3) c2(XP_011530603) c3(caspase-3 isoform X1) c4(326515, 326516, 326517, 326518, 326519) c5(326520, 326521, 326522, 326523, 326524, 326525, 326526, 326527, 326528, 326529, 326530, 326531, 326532, 326533, 326534, 326535, 326536, 326537, 326538, 326539, 326540, 326541, 326542, 326543, 326544, 326545, 326546, 326547, 326548, 326549, 326550, 326551, 326552, 326553, 326554, 326555, 326556, 326557, 326558, 326559, 326560, 326561, 326562, 326563, 326564, 326565, 326566, 326567, 326568, 326569, 326570, 326571, 326572, 326573, 326574, 326575, 326576, 326577, 326578, 326579, 326580, 326581, 326582, 326583, 326584, 326585, 326586, 326587, 326588, 326589, 326590, 326591, 326592, 326593, 326594, 326595, 326596, 326597, 326598, 326599, 326600, 326601, 326602, 326603, 326604, 326605, 326606, 326607, 326608, 326609, 326610, 326611, 326612, 326613, 326614, 326615, 326616, 326617, 326618, 326619, 326620, 326621, 326622, 326623, 326624, 326625, 326626, 326627, 326628, 326629, 326630, 326631, 326632, 326633, 326634, 326635, 326636, 326637, 326638, 326639, 326640, 326641, 326642, 326643, 326644, 326645, 326646, 326647, 326648, 326649, 326650, 326651, 326652, 326653, 326654, 326655, 326656, 326657, 326658, 326659, 326660, 326661, 326662, 326663, 326664, 326665, 326666, 326667, 326668, 326669, 326670, 326671, 326672, 326673, 326674, 326675, 326676, 326677, 326678, 326679, 326680, 326681, 326682, 326683, 326684, 326685, 326686, 326687, 326688, 326689, 326690, 326691, 326692, 326693, 326694, 326695, 326696, 326697, 326698, 326699, 326700, 326701, 326702, 326703, 326704, 326705, 326706, 326707, 326708, 326709, 326710, 326711, 326712, 326713, 326714, 326715, 326716, 326717, 326718) c6( ) c7(gh, T, os, fR, apr, if, fd, bB, ci, akg, aAo, Ul, aR, ip, x, XW, bv, zi, aE, Bc, fm, us, K, hu, zy, H, pk, dF, eL, Qe, up, aAu, aW, fc, XV, hN, g, eE, eF, cm, bw, iP, y, eM, vA, mx, aiM, cj, U, vy, zv, k, m, lS, hh, fy, in, iu, Ro, dD, FG, sw, ht, lb, n, aj, fr, asF, bd, PJ, qN, cd, eW, ft, jz, Yc, pN, kH, h, lG, eV, dL, bl, yZ, Dz, A, aJ, EU, CM, hl, aq, yq, Rl, dw, ajz, alj, cM, fs, op, Ld, Ye, ar, bP, ky, dB, hk, Qu, lf, ajd, hl, at, uz, cB, B, L, bL, fM, dA, aAs, eX, rf, aA, hy, C, il, AO, V, q, J, eA, mj, Yn, uT, hb, HC, aT, aAn, cy, vH, rO, aAv, aAt, bq, an, jj, ae, af, eu, hM, iU, Y, ev, r, aAm, akY, en, cl, xl, t, kC, f, pb, ik, rY, dS, ck, vk, Ta, rR, iM, ac, c, kB, gX, adR, aAp, bt, rA, LS, ao, hl, kh, id, qb, iW, jh, oZ, eq, aoz, ad, aAq, hX, av, hd, F, ib, sO, ds, NS, nl, oW, abP, lC, vc, fb, d, lR, je, hG, ff, eH, rU, eZ, akL, aF, bz, aAr, Hv, ek, iq, LB, bF, DT, bE, Ny, aS, cP, hE, KS, X, Ex, Tc); #c1(CASP4) c2(NP_001216) c3(caspase-4 isoform alpha precursor) c4(83790, 83791, 83792, 83793) c5(83794, 83795, 83796, 83797, 83798, 83799, 83800, 83801, 83802, 83803, 83804, 83805, 83806) c6( ) c7(T, fb, id, af, aW, Y, iW, aT, BF, K, aAw, PJ, ci, fy, bz, NM, q, ad, rY, g); #c1(CASP4) c2(NP_150649) c3(caspase-4 isoform gamma precursor) c4( ) c5(228261, 228262, 228263, 228264, 228265, 228266, 228267, 228268, 228269, 228270, 228271) c6( ) c7(T, fb, id, af, aW, Y, iW, aT, BF, K, aAw, PJ, ci, fy, bz, NM, q, ad, rY, g); #c1(CASP5) c2(NP_001129581) c3(caspase-5 isoform b precursor) c4(51872) c5(51873, 51874, 51875, 51876, 51877, 51878) c6( ) c7(T, iW, jh, ci, V, q, ad, av, F, aT, fb, aW, g, t, bz, ck, es, bP, iA, gX, cj, fy); #c1(CASP5) c2(NP_001129582) c3(caspase-5 isoform c) c4( ) c5(51879, 51880, 51881, 51882, 51883, 51884, 51885, 51886, 51887, 51888, 51889) c6( ) c7(T, iW, jh, ci, V, q, ad, av, F, aT, fb, aW, g, t, bz, ck, es, bP, iA, gX, cj, fy); #c1(CASP5) c2(NP_001129584) c3(caspase-5 isoform f precursor) c4(51890, 51891) c5(51892, 51893, 51894, 51895, 51896, 51897, 51898, 51899, 51900, 51901) c6( ) c7(T, iW, jh, ci, V, q, ad, av, F, aT, fb, aW, g, t, bz, ck, es, bP, iA, gX, cj, fy); #c1(CASP5) c2(NP_004338) c3(caspase-5 isoform a precursor) c4(145472, 145473) c5(145474, 145475, 145476, 145477, 145478, 145479) c6( ) c7(T, iW, jh, ci, V, q, ad, av, F, aT, fb, aW, g, t, bz, ck, es, bP, iA, gX, cj, fy); #c1(CASP5) c2(XP_011541323) c3(caspase-5 isoform X2) c4( ) c5(339918, 339919, 339920, 339921, 339922) c6( ) c7(T, iW, jh, ci, V, q, ad, av, F, aT, fb, aW, g, t, bz, ck, es, bP, iA, gX, cj, fy); #c1(CASP6) c2(NP_001217) c3(caspase-6 isoform alpha preproprotein) c4(83807, 83808) c5(83809, 83810, 83811, 83812, 83813, 83814, 83815, 83816, 83817, 83818, 83819, 83820, 83821, 83822, 83823, 83824, 83825, 83826, 83827, 83828, 83829, 83830, 83831, 83832, 83833, 83834, 83835, 83836, 83837, 83838, 83839, 83840, 83841, 83842, 83843, 83844, 83845, 83846, 83847, 83848, 83849, 83850, 83851, 83852, 83853, 83854, 83855, 83856, 83857, 83858, 83859, 83860, 83861, 83862, 83863, 83864, 83865, 83866, 83867, 83868, 83869, 83870, 83871, 83872, 83873, 83874, 83875, 83876, 83877, 83878, 83879, 83880, 83881, 83882, 83883, 83884, 83885, 83886, 83887, 83888, 83889, 83890, 83891, 83892, 83893, 83894, 83895, 83896, 83897, 83898, 83899, 83900, 83901, 83902, 83903) c6( ) c7(T, fM, sw, iW, aA, bB, ci, C, eA, ad, abg, h, lG, bq, kU, fb, af, eu, Y, lh, CW, r, g, iK, Bv, rR, ac, bE, U, cB, X); #c1(CASP6) c2(NP_116787) c3(caspase-6 isoform beta) c4(226488) c5(226489, 226490, 226491, 226492, 226493, 226494) c6( ) c7(T, fM, sw, iW, aA, bB, ci, C, eA, ad, abg, h, lG, bq, kU, fb, af, eu, Y, lh, CW, r, g, ik, Bv, rR, ac, bE, U, cB, X); #c1(CASP7) c2(NP_001218) c3(caspase-7 isoform alpha precursor) c4(83904, 83905, 83906) c5(83907, 83908, 83909, 83910, 83911, 83912, 83913, 83914, 83915, 83916, 83917, 83918, 83919, 83920, 83921, 83922, 83923, 83924, 83925, 83926, 83927) c6( ) c7(T, pf, sw, iW, jh, aA, n, hy, qj, ci, V, q, ad, eW, Bc, aT, lG, K, fb, ae, af, A, B, BN, iU, JI, aW, Y, rU, r, aq, aF, g, cl, ik, bz, eM, dJ, hL, y, bt, ne); #c1(CASP7) c2(NP_001253986) c3(caspase-7 isoform e) c4( ) c5(94362, 94363, 94364) c6( ) c7(T, pf, sw, iW, jh, aA, n, hy, qj, ci, V, q, ad, eW, Bc, aT, IG, K, fb, ae, af, A, B, BN, iU, JI, aW, Y, rU, r, aq, aF, g, cl, ik, bz, eM, dJ, hL, y, bt, ne); #c1(CASP7) c2(NP_001253987) c3(caspase-7 isoform f) c4(94365) c5(94366, 94367, 94368, 94369, 94370, 94371) c6( ) c7(T, pf, sw, iW, jh, aA, n, hy, qj, ci, V, q, ad, eW, Bc, aT, lG, K, fb, ae, af, A, B, BN, iU, JI, aW, Y, rU, r, aq, aF, g, cl, ik, bz, eM, dJ, hL, y, bt, ne); #c1(CASP7) c2(NP_203124) c3(caspase-7 isoform delta) c4(228315) c5(228316, 228317, 228318, 228319, 228320, 228321, 228322, 228323, 228324, 228325, 228326) c6( ) c7(T, pf, sw, iW, jh, aA, n, hy, qj, ci, V, q, ad, eW, Bc, aT, lG, K, fb, ae, af, A, B, BN, iU, JI, aW, Y, rU, r, aq, aF, g, cl, ik, bz, eM, dJ, hL, y, bt, ne); #c1(CASP7) c2(NP_203126) c3(caspase-7 isoform beta) c4(228327, 228328, 228329) c5(228330, 228331, 228332, 228333, 228334, 228335, 228336, 228337, 228338, 228339, 228340, 228341, 228342, 228343, 228344, 228345, 228346, 228347) c6( ) c7(T, pf, sw, iW, jh, aA, n, hy, qj, ci, V, q, ad, eW, Bc, aT, lG, K, fb, ae, af, A, B, BN, iU, JI, aW, Y, rU, r, aq, aF, g, cl, ik, bz, eM, dJ, hl, y, bt, ne); #c1(CASP8) c2(NP_001073594) c3(caspase-8 isoform G precursor) c4(36081, 36082) c5(36083, 36084, 36085, 36086, 36087, 36088, 36089, 36090) c6( ) c7(bM, T, os, aC, fR, pf, hV, if, aAx, fd, bB, ci, Qp, ip, x, XW, bv, aE, Bc, Qk, Ou, K, eL, eG, zQ, aW, az, g, pl, eE, lk, eF, cm, mk, aAB, eM, nB, aiM, cj, U, k, m, gn, hh, gU, pG, aAz, aw, ht, NT, n, aj, fr, qN, eW, ft, h, dL, hB, pi, sp, fJ, aq, yq, cM, Zj, bP, dB, hk, hL, at, fl, cB, B, aAE, ek, it, bL, fM, dA, jx, aA, hy, C, V, q, er, eA, mj, cF, aAF, aT, aAn, kf, cy, bK, aAD, jj, bq, aAC, ae, af, ne, jf, s, nO, iU, oV, Y, xR, r, lo, en, cl, t, ik, ck, ahN, rR, nM, Dy, c, ac, iM, wR, ro, im, bt, LS, pY, iW, aAk, jh, KO, oZ, mC, ad, av, hd, db, io, hD, ji, fb, nJ, Yi, aAy, aca, d, BN, aAA, je, ff, eH, eZ, Ej, aF, bz, eU, bE, Rv, Ny, hE, kS, X, y); #c1(CASP8) c2(NP_001219) c3(caspase-8 isoform A precursor) c4(83928) c5(83929, 83930, 83931, 83932, 83933, 83934) c6( ) c7(bM, T, os, aC, fR, pf, hV, if, aAx, fd, bB, ci, Qp, ip, x, XW, bv, aE, Bc, Ok, Ou, K, eL, eG, zQ, aW, az, g, pl, eE, lk, eF, cm, mk, aAB, eM, nB, aiM, cj, U, k, m, gn, hh, gU, pG, aAz, aw, ht, NT, n, aj, fr, qN, eW, ft, h, dL, hB, pi, sp, fJ, aq, yq, cM, Zj, bP, dB, hk, hL, at, fL, cB, B, aAE, ek, it, bL, fM, dA, jx, aA, hy, C, V, q, er, eA, mj, cF, aAF, aT, aAn, kf, cy, bK, aAD, jj, bq, aAC, ae, af, ne, jf, s, no, iU, oV, Y, xR, r, lo, en, cl, t, ik, ck, ahN, rR, nM, Dy, c, ac, iM, wR, ro, im, bt, LS, pY, iW, aAk, jh, KO, oZ, mC, ad, av, hd, db, io, hD, ji, fb, nJ, Yi, aAy, aca, d, BN, aAA, je, ff, eH, eZ, Ej, aF, bz, eU, bE, Rv, Ny, hE, KS, X, y); #c1(CASP8) c2(NP_203519) c3(caspase-8 isoform B precursor) c4(228356) c5(228357, 228358, 228359, 228360, 228361) c6( ) c7(bM, T, os, aC, fR, pf, hV, if, aAx, fd, bB, ci, Ap, ip, x, XW, bv, aE, Bc, Qk, Ou, K, eL, eG, zQ, aW, az, g, pl, BE, lk, eF, cm, mK, aAB, eM, nB, aiM, cj, U, k, m, gn, hh, gU, pG, aAz, aw, ht, NT, n, aj, fr, qN, eW, ft, h, dL, hB, pi, sp, fJ, aq, yq, cM, Zj, bP, dB, hk, hl, at, fL, cB, B, aAE, ek, it, bL, fM, dA, jx, aA, hy, C, V, q, er, eA, mj, cF, aAF, aT, aAn, kf, cy, bK, aAD, jj, bq, aAC, ae, af, ne, jf, s, nO, iU, oV, Y, xR, r, lo, en, cl, t, ik, ck, ahN, rR, nM, Dy, c, ac, iM, wR, ro, im, bt, LS, pY, iW, aAk, jh, KO, oZ, mC, ad, av, hd, db, io, hD, ji, fb, nJ, Yi, aAy, aca, d, BN, aAA, je, ff, eH, eZ, Ej, aF, bz, eU, bE, Rv, Ny, hE, KS, X, y); #c1(CASP8) c2(NP_203520) c3(caspase-8 isoform C precursor) c4(228362, 228363) c5(228364, 228365, 228366, 228367, 228368, 228369, 228370, 228371, 228372, 228373, 228374, 228375, 228376) c6( ) c7(bM, T, os, aC, fR, pf, hV, if, aAx, fd, bB, ci, Op, ip, x, XW, bv, aE, Bc, Qk, Ou, K, eL, eG, zQ, aW, az, g, pl, BE, lk, eF, cm, mK, aAB, eM, nB, aiM, cj, U, k, m, gn, hh, gU, pG, aAz, aw, ht, NT, n, aj, fr, qN, eW, ft, h, dL, hB, pi, sp, fJ, aq, yq, cM, Zj, bP, dB, hk, hL, at, fL, cB, B, aAE, ek, it, bL, fM, dA, jx, aA, hy, C, V, q, er, eA, mj, cF, aAF, aT, aAn, kf, cy, bK, aAD, jj, bq, aAC, ae, af, ne, jf, s, nO, iU, oV, Y, xR, r, lo, en, cl, t, ik, ck, ahN, rR, nM, Dy, c, ac, iM, wR, ro, im, bt, LS, pY, iW, aAk, jh, KO, oZ, mC, ad, av, hd, db, io, hD, ji, fb, nJ, Yi, aAy, aca, d, BN, aAA, je, ff, eH, eZ, Ej, af, bz, eU, bE, Rv, Ny, HE, KS, X, y); #c1(CASP8) c2(NP_203522) c3(caspase-8 isoform E) c4(228377) c5(228378, 228379, 228380, 228381, 228382, 228383, 228384, 228385, 228386, 228387, 228388, 228389, 228390, 228391, 228392, 228393, 228394, 228395, 228396, 228397, 228398, 228399, 228400, 228401) c6( ) c7(bM, T, os, aC, fR, pf, hV, if, aAx, fd, bB, ci, Ap, ip, x, XW, bv, aE, Bc, Qk, Ou, K, eL, eG, zQ, aW, az, g, pl, eE, lk, eF, cm, mK, aAB, eM, nB, aiM, cj, U, k, m, gn, hh, gU, pG, aAz, aw, ht, NT, n, aj, fr, qN, eW, ft, h, dL, hB, pi, sp, fJ, aq, yq, cM, Zj, bP, dB, hk, hL, at, fL, cB, B, aAE, ek, it, bL, fM, dA, jx, aA, hy, C, V, q, er, eA, mj, cF, aAF, aT, aAn, kf, cy, bK, aAD, jj, bq, aAC, ae, af, ne, jf, s, nO, iU, oV, Y, xR, r, lo, en, cl, t, ik, ck, ahN, rR, nM, Dy, c, ac, iM, wR, ro, im, bt, LS, pY, iW, aAk, jh, KO, oZ, mC, ad, av, hd, db, io, hD, ji, fb, nJ, Yi, aAy, aca, d, BN, aAA, je, ff, eH, eZ, Ej, aF, bz, eU, bE, Rv, Ny, HE, KS, X, y); #c1(CASP8) c2(XP_005246942) c3(caspase-8 isoform X4) c4(267534) c5(267535, 267536, 267537, 267538) c6( ) c7(bM, T, os, aC, fR, pf, hV, if, aAx, fd, bB, ci, Ap, ip, x, XW, bv, aE, Bc, Qk, Ou, K, eL, eG, zQ, aW, az, g, pl, eE, lk, eF, cm, mK, aAB, eM, nB, aiM, cj, U, k, m, gn, hh, gU, pG, aAz, aw, ht, NT, n, aj, fr, qN, eW, ft, h, dL, hB, pi, sp, fJ, aq, yq, cM, Zj, bP, dB, hk, hL, at, fL, cB, B, aAE, ek, it, bL, fM, dA, jx, aA, hy, C, V, q, er, eA, mj, cF, aAF, aT, aAn, kf, cy, bK, aAD, jj, bq, aAC, ae, af, ne, jf, s, nO, iU, oV, Y, xR, r, lo, en, cl, t, ik, ck, ahN, rR, nM, Dy, c, ac, iM, wR, ro, im, bt, LS, pY, iW, aAk, jh, KO, oZ, mC, ad, av, hd, db, io, HD, ji, fb, nJ, Yi, aAy, aca, d, BN, aAA, je, ff, eH, eZ, Ej, aF, bz, eU, bE, Rv, Ny, HE, KS, X, y); #c1(CASP8) c2(XP_005246952) c3(caspase-8 isoform X10) c4(267539, 267540) c5(267541, 267542, 267543, 267544, 267545, 267546, 267547, 267548, 267549, 267550, 267551, 267552) c6( ) c7(bM, T, os, aC, fR, pf, hV, if, aAx, fd, bB, ci, Qp, ip, x, XW, bv, aE, Bc, Qk, Ou, K, eL, eG, zQ, aW, az, g, pl, eE, lk, eF, cm, mk, aAB, eM, nB, aiM, cj, U, k, m, gn, hh, gU, pG, aAz, aw, ht, NT, n, aj, fr, qN, eW, ft, h, dL, hB, pi, sp, fJ, aq, yq, cM, Zj, bP, dB, hk, hl, at, fL, cB, B, aAE, ek, it, bL, fM, dA, jx, aA, hy, C, V, q, er, eA, mj, cF, aAF, aT, aAn, kf, cy, bK, aAD, jj, bq, aAC, ae, af, ne, jf, s, nO, iU, oV, Y, xR, r, lo, en, cl, t, iK, ck, ahN, rR, nM, Dy, c, ac, iM, wR, ro, im, bt, LS, pY, iW, aAk, jh, KO, oZ, mC, ad, av, hd, db, io, hD, ji, fb, nJ, Yi, aAy, aca, d, BN, aAA, je, ff, eH, eZ, Ej, aF, bz, eU, bE, Rv, Ny, hE, KS, X, y); #c1(CASP8) c2(XP_006712854) c3(caspase-8 isoform X5) c4(286895) c5(286896, 286897, 286898, 286899, 286900, 286901) c6( ) c7(bM, T, os, aC, fR, pf, hV, if, aAx, fd, bB, ci, Qp, ip, x, XW, bv, aE, Bc, Qk, Ou, K, eL, eG, zQ, aW, az, g, pl, eE, lk, eF, cm, mk, aAB, eM, nB, aiM, cj, U, k, m, gn, hh, gU, pG, aAz, aw, ht, NT, n, aj, fr, qN, eW, ft, h, dL, hB, pi, sp, fJ, aq, yq, cM, Zj, bP, dB, hk, hL, at, fL, cB, B, aAE, ek, it, bL, fM, dA, jx, aA, hy, C, V, q, er, eA, mj, cF, aAF, aT, aAn, kf, cy, bK, aAD, jj, bq, aAC, ae, af, ne, jf, s, nO, iU, oV, Y, xR, r, lo, en, cl, t, ik, ck, ahN, rR, nM, Dy, c, ac, iM, wR, ro, im, bt, LS, pY, iW, aAk, jh, KO, oZ, mC, ad, av, hd, db, io, hD, ji, fb, nJ, Yi, aAy, aca, d, BN, aAA, je, ff, eH, eZ, Ej, aF, bz, eU, bE, Rv, Ny, hE, kS, X, y); #c1(CASP8) c2(XP_011510272) c3(caspase-8 isoform X8) c4(300868) c5(300869, 300870) c6( ) c7(bM, T, os, aC, fR, pf, hV, if, aAx, fd, bB, ci, Ap, ip, x, XW, bv, aE, Bc, Qk, Ou, K, eL, eG, zQ, aW, az, g, pl, eE, lk, eF, cm, mk, aAB, eM, nB, aiM, cj, U, k, m, gn, hh, gU, pG, aAz, aw, ht, NT, n, aj, fr, qN, eW, ft, h, dL, hB, pi, sp, fJ, aq, yq, cM, Zj, bP, dB, hk, hL, at, fL, cB, B, aAE, ek, it, bL, fM, dA, jx, aA, hy, C, V, q, er, eA, mj, cF, aAF, aT, aAn, kf, cy, bK, aAD, jj, bq, aAC, ae, af, ne, jf, s, nO, iU, oV, Y, xR, r, lo, en, cl, t, iK, ck, ahN, rR, nM, Dy, c, ac, iM, wR, ro, im, bt, LS, pY, iW, aAk, jh, KO, oZ, mC, ad, av, hd, db, io, hD, ji, fb, nJ, Yi, aAy, aca, d, BN, aAA, je, ff, eH, eZ, Ej, aF, bz, eU, bE, Rv, Ny, hE, kS, X, y); #c1(CASP8AP2) c2(NP_001131140) c3(CASP8-associated protein 2) c4(52606) c5(52607, 52608, 52609, 52610, 52611, 52612, 52613, 52614) c6( ) c7(oL, T, fX, vT, iW, r, h, ci, XU, aAG, q); #c1(CASP9) c2(XP_011540572) c3(caspase-9 isoform X2) c4( ) c5(339058, 339059, 339060, 339061, 339062, 339063) c6( ) c7(bL, T, aC, fM, fR, pf, sw, if, iW, jx, jh, aA, lD, n, fd, bS, fr, ci, C, bX, qN, zP, V, asr, q, aR, ad, eA, eW, XW, av, jz, aE, Bc, aT, ab, lG, nm, cy, dL, K, hD, bq, an, fb, ae, eL, eu, jf, d, iU, vT, aW, Y, eH, hl, r, vs, aF, g, xl, t, eF, bz, cm, ik, vk, ac, c, eM, asq, aiM, cj, gZ, U, at, m, bC, bt, ik, B, Ex, rp); #c1(CASP9) c2(XP_011540574) c3(caspase-9 isoform X1) c4( ) c5(339064, 339065, 339066, 339067, 339068, 339069, 339070, 339071, 339072, 339073, 339074, 339075, 339076, 339077, 339078) c6( ) c7(bL, T, aC, fM, fR, pf, sw, if, iW, jx, jh, aA, lD, n, fd, bS, fr, ci, C, bX, qN, zP, V, asr, q, aR, ad, eA, eW, XW, av, jz, aE, Bc, aT, ab, lG, nm, cy, dL, K, hD, bq, an, fb, ae, eL, eu, jf, d, iU, vT, aW, Y, eH, hl, r, vs, aF, g, xl, t, eF, bz, cm, ik, vk, ac, c, eM, asq, aiM, cj, gZ, U, at, m, bC, bt, ik, B, Ex, rp); #c1(CASP9) c2(XP_011540575) c3(caspase-9 isoform X3) c4(339079) c5(339080, 339081, 339082, 339083, 339084, 339085) c6( ) c7(bL, T, aC, fM, fR, pf, sw, if, iW, jx, jh, aA, lD, n, fd, bS, fr, ci, C, bX, qN, zP, V, asr, q, aR, ad, eA, eW, XW, av, jz, aE, Bc, aT, ab, lG, nm, cy, dL, K, hD, bq, an, fb, ae, eL, eu, jf, d, iU, vT, aW, Y, eH, hl, r, vs, aF, g, xl, t, eF, bz, cm, ik, vk, ac, c, eM, asq, aiM, cj, gZ, U, at, m, bC, bt, ik, B, Ex, rp); #c1(CASQ1) c2(NP_001222) c3(calsequestrin-1 precursor) c4(83942, 83943, 83944) c5(83945, 83946, 83947, 83948, 83949, 83950, 83951, 83952, 83953, 83954, 83955, 83956, 83957, 83958, 83959, 83960, 83961, 83962) c6( ) c7(bL, ac, mP, lW, aj, mf, K, Jk, bq, J); #c1(CASR) c2(NP_001171536) c3(extracellular calcium-sensing receptor isoform 1 precursor) c4(70547) c5(70548, 70549, 70550, 70551, 70552, 70553) c6( ) c7(T, ea, al, fd, bB, ci, aY, aE, Bc, eL, azh, yo, PP, aW, aAT, Lm, dg, as, eF, cm, TG, mx, wB, cj, iS, azg, m, lS, aAK, dD, aAP, aw, NT, n, aj, fr, ml, eW, cc, afV, dL, aAG, OM, aAl, OX, aAH, cM, ar, kT, YE, Qu, at, eo, aAQ, B, vQ, bL, aAM, dA, eR, cz, v, j, ON, sl, q, J, eA, Ql, mj, TI, XD, nv, aT, aAR, bq, ae, af, bA, s, oV, Y, EK, r, lo, t, ck, c, dy, gT, bt, aAD, gV, pY, qb, ha, jh, eq, ad, Xv, fU, hd, LL, arV, yh, aAL, sj, ul, hG, vj, eH, oO, dq, HI, bz, gk, Cq, aAS, bE, AR, avl, MJ, aAJ, lC, aAN, X); #c1(CASR) c2(XP_005247893) c3(extracellular calcium-sensing receptor isoform X1) c4(268021) c5(268022, 268023, 268024, 268025, 268026, 268027, 268028, 268029, 268030, 268031) c6( ) c7(T, ea, al, fd, bB, ci, aY, aE, Bc, eL, azh, yo, PP, aW, aAT, Lm, dg, as, eF, cm, TG, mx, wB, cj, iS, azg, m, lS, aAK, dD, aAP, aw, NT, n, aj, fr, ml, eW, cc, afV, dL, aAG, OM, aAl, OX, aAH, cM, ar, KT, YE, Qu, at, eo, aAQ, B, vQ, bL, aAM, dA, eR, cz, v, j, ON, sl, q, J, eA, QI, mj, TI, XD, nv, aT, aAR, bq, ae, af, bA, s, oV, Y, EK, r, lo, t, ck, c, dy, gT, bt, aAD, gV, pY, qb, ha, jh, eq, ad, Xv, fU, hd, LL, arV, yh, aAL, sj, ul, hG, vj, eH, oO, dq, HI, bz, gk, Cq, aAS, bE, AR, avl, MJ, aAJ, lC, aAN, X); #c1(CASR) c2(XP_005247894) c3(extracellular calcium-sensing receptor isoform X2) c4(268032) c5( ) c6( ) c7(T, ea, al, fd, bB, ci, aY, aE, Bc, eL, azh, yo, PP, aW, aAT, Lm, dg, as, eF, cm, TG, mx, wB, cj, iS, azg, m, lS, aAK, dD, aAP, aw, NT, n, aj, fr, ml, eW, cc, afV, dL, aAG, OM, aAl, OX, aAH, cM, ar, kT, YE, Qu, at, eo, aAQ, B, vQ, bL, aAM, dA, eR, cz, v, j, ON, sl, q, J, eA, QI, mj, TI, XD, nv, aT, aAR, bq, ae, af, bA, s, oV, Y, EK, r, lo, t, ck, c, dy, gT, bt, aAD, gV, pY, qb, ha, jh, eq, ad, Xv, fU, hd, LL, arV, yh, aAL, sj, ul, hG, vj, eH, oO, dq, HI, bz, gk, Cq, aAS, bE, AR, avl, MJ, aAJ, lC, aAN, X); #c1(CASS4) c2(NP_001157587) c3(cas scaffolding protein family member 4 isoform c) c4(62304) c5(62305, 62306, 62307, 62308, 62309, 62310) c6( ) c7(T); #c1(CASS4) c2(NP_001157588) c3(cas scaffolding protein family member 4 isoform a) c4(62311) c5(62312, 62313, 62314, 62315, 62316, 62317, 62318, 62319, 62320, 62321) c6( ) c7(T); #c1(CAST) c2(NP_001035905) c3(calpastatin isoform f) c4(33369) c5(33370, 33371, 33372, 33373, 33374, 33375) c6( ) c7(Kd, dD, ea, fG, eX, jh, cz, v, eq, C, uE, V, q, aR, mC, cd, Bc, aT, h, lG, cy, eV, bl, ji, an, zy, H, af, eu, akX, d, aW, Fm, Y, FD, cr, eZ, fe, t, cm, Hv, iO, VJ, ac, c, hc, wB, U, uo, nw, eo, m, fl, aAU, y, X, hv, L); #c1(CAST) c2(NP_001177371) c3(calpastatin isoform m) c4(72716) c5(72717, 72718, 72719, 72720, 72721) c6( ) c7(Kd, dD, ea, fG, eX, jh, cz, v, eq, C, uE, V, q, aR, mC, cd, Bc, aT, h, lG, cy, eV, bl, ji, an, zy, H, af, eu, akX, d, aW, Fm, Y, FD, cr, eZ, fe, t, cm, Hv, iO, VJ, ac, c, hc, wB, U, uo, nw, eo, m, fl, aAU, y, X, hv, L); #c1(CAST) c2(NP_775083) c3(calpastatin isoform b) c4(249488) c5(249489, 249490, 249491, 249492, 249493, 249494) c6( ) c7(Kd, dD, ea, fG, eX, jh, cz, v, eq, C, uE, V, q, aR, mC, cd, Bc, aT, h, lG, cy, eV, bl, ji, an, zy, H, af, eu, akX, d, aW, Fm, Y, FD, cr, eZ, fe, t, cm, Hv, iO, VJ, ac, c, hc, wB, U, uo, nw, eo, m, fl, aAU, y, X, hv, L); #c1(CAST) c2(XP_006714759) c3(calpastatin isoform X2) c4(288105) c5(288106, 288107, 288108, 288109, 288110, 288111, 288112, 288113, 288114, 288115) c6( ) c7(Kd, dD, ea, fG, eX, jh, cz, v, eq, C, uE, V, q, aR, mC, cd, Bc, aT, h, lG, cy, eV, bl, ji, an, zy, H, af, eu, akX, d, aW, Fm, Y, FD, cr, eZ, fe, t, cm, Hv, iO, VJ, ac, c, hc, wB, U, uo, nw, eo, m, fl, aAU, y, X, hv, L); #c1(CAST) c2(XP_006714761) c3(calpastatin isoform X4) c4( ) c5(288116) c6( ) c7(Kd, dD, ea, fG, eX, jh, cz, v, eq, C, uE, V, q, aR, mC, cd, Bc, aT, h, lG, cy, eV, bl, ji, an, zy, H, af, eu, akX, d, aW, Fm, Y, FD, cr, eZ, fe, t, cm, Hv, iO, VJ, ac, c, hc, wB, U, uo, nw, eo, m, fl, aAU, y, X, hv, L); #c1(CAST) c2(XP_006714762) c3(calpastatin isoform X5) c4(288117, 288118, 288119) c5(288120) c6( ) c7(Kd, dD, ea, fG, eX, jh, cz, v, eq, C, uE, V, q, aR, mC, cd, Bc, aT, h, lG, cy, eV, bl, ji, an, zy, H, af, eu, akX, d, aW, Fm, Y, FD, cr, eZ, fe, t, cm, Hv, iO, VJ, ac, c, hc, wB, U, uo, nw, eo, m, fl, aAU, y, X, hv, L); #c1(CAST) c2(XP_006714763) c3(calpastatin isoform X6) c4(288121) c5(288122, 288123, 288124, 288125, 288126, 288127) c6( ) c7(Kd, dD, ea, fG, eX, jh, cz, v, eq, C, uE, V, q, aR, mC, cd, Bc, aT, h, lG, cy, eV, bl, ji, an, zy, H, af, eu, akX, d, aW, Fm, Y, FD, cr, eZ, fe, t, cm, Hv, iO, VJ, ac, c, hc, wB, U, uo, nw, eo, m, fL, aAU, y, X, hv, L); #c1(CAST) c2(XP_006714766) c3(calpastatin isoform X11) c4(288128) c5(288129, 288130, 288131, 288132, 288133, 288134) c6( ) c7(Kd, dD, ea, fG, eX, jh, cz, v, eq, C, uE, V, q, aR, mC, cd, Bc, aT, h, lG, cy, eV, bl, ji, an, zy, H, af, eu, akX, d, aW, Fm, Y, FD, cr, eZ, fe, t, cm, Hv, iO, VJ, ac, c, hc, wB, U, uo, nw, eo, m, fl, aAU, y, X, hv, L); #c1(CAST) c2(XP_006714767) c3(calpastatin isoform X12) c4(288135) c5(288136, 288137, 288138, 288139, 288140, 288141) c6( ) c7(Kd, dD, ea, fG, eX, jh, cz, v, eq, C, uE, V, q, aR, mC, cd, Bc, aT, h, IG, cy, eV, bl, ji, an, zy, H, af, eu, akX, d, aW, Fm, Y, FD, cr, eZ, fe, t, cm, Hv, iO, VJ, ac, c, hc, wB, U, uo, nw, eo, m, fl, aAU, y, X, hv, L); #c1(CAST) c2(XP_006714770) c3(calpastatin isoform X17) c4( ) c5(288142, 288143, 288144, 288145, 288146, 288147, 288148, 288149, 288150, 288151, 288152, 288153, 288154, 288155, 288156, 288157, 288158) c6( ) c7(Kd, dD, ea, fG, eX, jh, cz, v, eq, C, uE, V, q, aR, mC, cd, Bc, aT, h, lG, cy, eV, bl, ji, an, zy, H, af, eu, akX, d, aW, Fm, Y, FD, cr, eZ, fe, t, cm, Hv, iO, VJ, ac, c, hc, wB, U, uo, nw, eo, m, fl, aAU, y, X, hv, L); #c1(CASZ1) c2(XP_011539937) c3(zinc finger protein castor homolog 1 isoform X3) c4( ) c5(338290, 338291, 338292, 338293, 338294, 338295) c6( ) c7(T, qo, d, aAV, F, aW, Y, oe, oW, aT, cz, eb, jh, ur, K, eA, eg, cm, bz, B); #c1(CAT) c2(NP_001743) c3(catalase) c4(120675) c5(120676, 120677, 120678, 120679, 120680, 120681) c6( ) c7(bM, T, os, aC, lP, ea, if, e, bB, fo, Vd, ci, qj, aBm, fw, ku, aBb, aE, Bc, Pl, aBg, K, qD, hH, N, Zm, aa, aAX, aW, az, fc, CT, aBd, La, aoD, Ku, hJ, aBa, g, eF, cm, aBh, wT, aAW, qJ, yl, aBr, eM, nB, Gs, U, vy, wx, m, aBn, pw, aBt, dD, aBe, aBf, My, aw, RC, BH, aj, ko, nA, PJ, aBl, Rc, qN, cd, zo, eW, JB, ft, cc, pe, qg, cU, lG, h, dl, wb, on, pi, Gc, hi, rk, A, Cw, ap, ge, Ni, aq, QK, cv, ej, ar, iO, aBs, yf, W, lX, dB, hk, eD, mg, hR, at, oh, lB, cB, B, abW, bL, eR, yk, qQ, rf, aA, cR, cz, aBo, v, j, Kh, C, AD, V, BA, J, er, eJ, mj, uT, HJ, nv, afQ, MI, aT, cy, bK, aAD, bq, DX, lM, ae, af, dl, bA, eu, azU, wr, s, nO, aBq, Y, ajx, fO, ate, r, en, cl, t, f, iK, bN, hY, ck, akF, rR, nM, ac, c, kB, iM, mG, hr, wR, mf, dy, aBp, ao, hS, am, aBj, aD, iW, jh, Hn, acU, oZ, dG, eq, atK, aAY, ad, kL, hX, dP, fU, hd, ib, yt, ds, Hk, ts, ab, mL, ji, nJ, sj, ul, NP, uX, je, lR, anv, qt, ff, eH, cr, eZ, dN, dq, Mz, aF, iV, bz, aBi, qa, iq, xT, aAZ, yE, bE, aBc, uR, eB, atd, aBk, aS, bC, be, ei, y, X, jr, pC); #c1(CATSPER1) c2(NP_444282) c3(cation channel sperm-associated protein 1) c4(230288) c5(230289, 230290, 230291, 230292, 230293, 230294, 230295) c6( ) c7(gU, aBu); #c1(CATSPER2) c2(NP_001269238) c3(cation channel sperm-associated protein 2 isoform 1) c4(100849) c5(100850, 100851, 100852, 100853, 100854, 100855, 100856, 100857, 100858, 100859, 100860, 100861, 100862, 100863, 100864, 100865, 100866, 100867, 100868, 100869, 100870, 100871, 100872) c6( ) c7(gU); #c1(CATSPER2) c2(NP_001269239) c3(cation channel sperm-associated protein 2 isoform 5) c4(100873) c5(100874, 100875, 100876, 100877, 100878) c6( ) c7(gU); #c1(CATSPER2) c2(NP_742093) c3(cation channel sperm-associated protein 2 isoform 2) c4(248069, 248070, 248071, 248072) c5(248073, 248074, 248075, 248076, 248077, 248078, 248079, 248080, 248081, 248082, 248083, 248084, 248085, 248086, 248087) c6( ) c7(gU); #c1(CAV1) c2(NP_001166366) c3(caveolin-1 isoform beta) c4( ) c5(68561, 68562, 68563, 68564, 68565, 68566, 68567, 68568) c6( ) c7(ID, T, aC, fR, ea, if, e, al, EQ, fd, bB, lR, ci, aY, ip, bv, aE, Qk, ajv, K, hu, eL, bc, cp, ly, eG, dH, JI, aW, az, LZ, vs, PQ, g, as, eF, GR, qJ, eM, nB, iA, go, cj, iS, m, Xu, fy, Th, iu, dD, Uc, My, cV, aBx, qy, n, aj, fr, uE, qN, qW, eW, aBy, ft, kH, h, IG, jy, dL, dx, wC, MD, hT, PX, aq, kp, dw, lx, bP, GU, dB, hj, at, uz, aBv, zl, it, B, bL, dA, eR, mr, wL, fG, aA, cR, cz, hy, v, j, C, AD, V, q, J, eA, XX, lg, LY, aah, aT, Oi, bK, bq, ae, af, ne, eu, qw, s, Y, ev, r, en, t, pb, iM, c, pZ, mG, nG, au, km, wR, np, fD, gT, aBw, bt, Kd, id, aZ, aBz, eq, Fc, iJ, ad, KL, BY, hX, av, iC, hd, QJ, oW, bj, nJ, d, BN, je, qt, ff, eH, aeV, aF, iR, bz, LB, es, gk, bF, bE, z, aS, ei, lC, X, hv); #c1(CAV1) c2(NP_001744) c3(caveolin-1 isoform alpha) c4(120682, 120683) c5(120684, 120685, 120686, 120687, 120688, 120689, 120690, (20691) c6( ) c7(ID, T, aC, fR, ea, if, e, al, EQ, fd, bB, lR, ci, aY, ip, bv, aE, Qk, ajv, K, hu, eL, bc, cp, ly, eG, dH, JI, aW, az, LZ, vs, PQ, g, as, eF, GR, qJ, eM, nB, iA, go, cj, iS, m, Xu, fy, Th, iu, dD, Uc, My, cV, aBx, qy, n, aj, fr, uE, qN, qW, eW, aBy, ft, kH, h, lG, jy, dL, dx, wC, MD, hT, PX, aq, kp, dw, lx, bP, GU, dB, hj, at, uz, aBv, zl, it, B, bL, dA, eR, mr, wL, fG, aA, cR, cz, hy, v, j, C, AO, V, q, J, eA, XX, lg, LY, aah, aT, Oi, bK, bq, ae, af, ne, eu, qw, s, Y, ev, r, en, t, pb, iM, c, pZ, mG, nG, au, km, wR, np, fD, gT, aBw, bt, Kd, id, aZ, aBz, eq, Fc, iJ, ad, KL, BY, hX, av, iC, hd, QJ, oW, bj, nJ, d, BN, je, qt, ff, eH, aeV, aF, iR, bz, LB, es, gk, bF, bE, z, aS, ei, lC, X, hv); #c1(CAV2) c2(NP_001224) c3(caveolin-2 isoform a) c4(83963, 83964) c5(83965, 83966, 83967, 83968, 83969, 83970, 83971, 83972, 83973, 83974, 83975, 83976) c6( ) c7(cZ, T, dD, fR, mr, wL, ha, al, hy, v, eq, ci, fr, qW, eA, ad, oW, aT, bK, dd, ne, bc, wC, d, BN, s, dH, aW, ff, eH, en, dw, eF, qJ, pZ, HN, Lq, X); #c1(CAV2) c2(NP_937855) c3(caveolin-2 isoform c) c4(259301) c5(259302, 259303, 259304, 259305, 259306, 259307) c6( ) c7(cZ, T, dD, fR, mr, wL, ha, al, hy, v, eq, ci, fr, qW, eA, ad, oW, aT, bK, dd, ne, bc, wC, d, BN, s, dH, aW, ff, eH, en, dw, eF, qJ, pZ, HN, Lq, X); #c1(CAV3) c2(NP_001225) c3(caveolin-3) c4(83977) c5(83978, 83979, 83980, 83981, 83982, 83983, 83984, 83985, 83986, 83987) c6( ) c7(aBD, AY, e, aBE, al, jN, aw, aeu, j, bX, pd, aBF, cc, no, fm, Ws, oj, aT, Ak, vc, bl, aBA, bq, HO, mu, wC, hM, xs, Ad, uY, EZ, avK, aBG, aBB, gY, mx, km, aBH, gZ, oh, bD, aBC); #c1(CBFA2T2) c2(NP_001028171) c3(protein CBFA2T2 isoform MTGR1c) c4(30330, 30331) c5(30332, 30333, 30334, 30335, 30336, 30337, 30338, 30339, 30340, 30341, 30342, 30343) c6( ) c7(T, oT, ac, af, cA, bE, aW, aZ, eM, aT, kH, aq, fd, ji, ci, ei, eA, bt, B); #c1(CBFA2T2) c2(XP_006723949) c3(protein CBFA2T2 isoform X1) c4(295552, 295553, 295554, 295555, 295556) c5(295557, 295558, 295559, 295560, 295561, 295562, 295563, 295564, 295565, 295566, 295567, 295568, 295569, 295570, 295571, 295572, 295573) c6( ) c7(T, oT, ac, af, cA, bE, aW, aZ, eM, aT, kH, aq, fd, ji, ci, ei, eA, bt, B); #c1(CBFA2T2) c2(XP_011527403) c3(protein CBFA212 isoform X3) c4(322118) c5(322119) c6( ) c7(T, oT, ac, af, cA, bE, aW, aZ, eM, aT, KH, aq, fd, ji, ci, ei, eA, bt, B); #c1(CBFA2T2) c2(XP_011527406) c3(protein CBFA2T2 isoform X2) c4( ) c5(322120, 322121, 322122, 322123, 322124, 322125, 322126, 322127, 322128, 322129, 322130) c6( ) c7(T, oT, ac, af, cA, bE, aW, aZ, eM, aT, kH, aq, fd, ji, ci, ei, eA, bt, B); #c1(CBFA2T2) c2(XP_011527407) c3(protein CBFA2T2 isoform X5) c4( ) c5(322131, 322132, 322133, 322134, 322135) c6( ) c7(T, oT, ac, af, cA, bE, aW, aZ, eM, aT, kH, aq, fd, ji, ci, ei, eA, bt, B); #c1(CBFA2T2) c2(XP_011527409) c3(protein CBFA2T2 isoform X6) c4( ) c5(322136, 322137, 322138, 322139, 322140, 322141) c6( ) c7(T, oT, ac, af, cA, bE, aW, aZ, eM, aT, kH, aq, fd, ji, ci, ei, eA, bt, B); #c1(CBFA2T3) c2(NP_005178) c3(protein CBFA213 isoform 1) c4(153650) c5(153651, 153652, 153653, 153654, 153655, 153656, 153657) c6( ) c7(ck, oB, T, fM, aW, jh, aT, iY, ji, K, iV, AO, bt); #c1(CBFA2T3) c2(XP_005256380) c3(protein CBFA2T3 isoform X1) c4(274138, 274139, 274140) c5(274141, 274142, 274143, 274144, 274145, 274146, 274147, 274148, 274149, 274150, 274151, 274152, 274153, 274154, 274155, 274156, 274157, 274158, 274159, 274160, 274161, 274162) c6( ) c7(ck, oB, T, fM, aW, jh, aT, iY, ji, K, iV, AO, bt); #c1(CBFB) c2(NP_001746) c3 (core-binding factor subunit beta isoform 2) c4(120713) c5(120714, 120715, 120716, 120717, 120718, 120719) c6( ) c7(aA, hF, cd, q, x, Na, fX, aT, h, ey, aBl, iU, aW, r, aF, g, Fu, t, ck, iT, aBJ, m, k, bD, bt); #c1(CBFB) c2(NP_074036) c3(core-binding factor subunit beta isoform 1) c4(213497) c5(213498, 213499, 213500, 213501, 213502, 213503) c6( ) c7(aA, hF, cd, q, x, Na, fx, aT, h, ey, aBl, iU, aW, r, aF, g, Fu, t, ck, iT, aBJ, m, k, bD, bt); #c1(CBL) c2(NP_005179) c3(E3 ubiquitin-protein ligase CBL) c4(153658) c5(153659, 153660, 153661, 153662, 153663, 153664) c6( ) c7(bL, T, fM, yS, qx, ew, bs, aA, jh, hF, mC, br, ad, amR, x, acA, KN, h, dz, fb, pq, dx, aBl, aBK, yi, oP, et, gK, eZ, kD, aF, pm, bw, Qt, yR, Cv, ck, pc, kz, gX, mg, pg, aBL, Km, k, fl, agy, bt, xb, hv); #c1(CBLB) c2(NP_733762) c3(E3 ubiquitin-protein ligase CBL-B) c4(246969, 246970, 246971) c5(246972, 246973, 246974, 246975, 246976, 246977, 246978, 246979, 246980, 246981, 246982, 246983, 246984, 246985, 246986, 246987, 246988, 246989) c6( ) c7(x, T, A, fO, hD, aj, eo, k, bl, jb, bt, V, J, uQ); #c1(CBLB) c2(XP_011511560) c3(E3 ubiquitin-protein ligase CBL-B isoform X2) c4(302278, 302279) c5(302280, 302281, 302282, 302283, 302284, 302285) c6( ) c7(x, T, A, fO, hD, aj, eo, k, bl, jb, bt, V, J, uQ); #c1(CBLB) c2(XP_011511561) c3(E3 ubiquitin-protein ligase CBL-B isoform X3) c4(302286) c5(302287, 302288, 302289, 302290, 302291, 302292, 302293, 302294, 302295, 302296, 302297, 302298) c6( ) c7(x, T, A, fO, hD, aj, eo, k, bl, jb, bt, V, J, uQ); #c1(CBLL1) c2(NP_001271220) c3(E3 ubiquitin-protein ligase Hakai isoform 2) c4(103641) c5(103642, 103643, 103644, 103645, 103646, 103647, 103648, 103649) c6( ) c7(lo, aiM); #c1(CBLL1) c2(NP_079090) c3(E3 ubiquitin-protein ligase Hakai isoform 1) c4(216961, 216962) c5(216963, 216964, 216965, 216966, 216967, 216968) c6( ) c7(lo, aiM); #c1(CBLL1) c2(XP_011514882) c3(E3 ubiquitin-protein ligase Hakai isoform X1) c4( ) c5(307633, 307634, 307635, 307636, 307637) c6( ) c7(lo, aiM); #c1(CBLN1) c2(NP_004343) c3(cerebellin-1 precursor) c4(145519) c5(145520, 145521, 145522, 145523, 145524, 145525, 145526, 145527, 145528, 145529, 145530, 145531, 145532, 145533) c6(145534, 145535, 145536, 145537) c7(Z, bS, ct, ad); #c1(CBLN2) c2(XP_011524128) c3(cerebellin-2 isoform X1) c4(318685) c5(318686, 318687, 318688, 318689, 318690, 318691) c6( ) c7(ID, ly, yL, lC, lx); #c1(CBLN4) c2(NP_542184) c3(cerebellin-4 precursor) c4(232255) c5(232256, 232257, 232258, 232259, 232260, 232261, 232262, 232263) c6(232264, 232265) c7(cm); #c1(CBR1) c2(NP_001748) c3(carbonyl reductase [NADPH] 1 isoform 1) c4(120734) c5(120735, 120736, 120737, 120738, 120739, 120740) c6( ) c7(T, hl, aA, bB, q, ad, jz, cc, nv, aT, h, dL, K, jj, A, B, aW, aBN, az, aBM, r, aF, iV, f, bz, ck, EW, bE, gX, at, lC, bt, X); #c1(CBR3) c2(NP_001227) c3(carbonyl reductase [NADPH] 3) c4(83988) c5(83989, 83990, 83991, 83992, 83993, 83994) c6( ) c7(bL, T, cc, aT, at, n, aeu, dL, K, t, bt, cd); #c1(CBR4) c2(NP_116172) c3(carbonyl reductase family member 4) c4(225550, 225551) c5(225552, 225553, 225554, 225555, 225556, 225557, 225558, 225559, 225560, 225561, 225562, 225563) c6( ) c7(aw); #c1(CBS) c2(XP_011528084) c3(cystathionine beta-synthase isoform X3) c4(323160) c5(323161, 323162, 323163, 323164, 323165, 323166) c6( ) c7(ng, T, FR, ea, e, al, fd, bB, ci, mF, aY, ip, Na, M, K, dF, eL, eN, aW, fc, g, eM, lp, wE, aBO, uo, m, dD, sQ, aw, aj, nA, aBP, aaQ, dL, aBR, ux, is, aBQ, wC, MX, sq, PX, fe, aq, kT, W, gY, dB, ayS, bm, at, ut, bD, B, bL, fM, aA, sM, CR, cz, v, j, C, q, J, aT, HG, Nc, an, ae, af, eu, Uf, oV, r, en, t, f, ck, rR, wg, ac, Nb, gX, fP, wR, bt, iW, jh, OR, eq, ad, av, iY, lQ, Zf, ff, eZ, dN, aF, bz, dr, ek, lU, bF, mO, bE, ag, gZ, aS, cP, Tw, us); #c1(CBX1) c2(NP_006798) c3(chromobox protein homolog 1) c4(168173, 168174) c5(168175, 168176, 168177, 168178, 168179, 168180, 168181, 168182, 168183, 168184, 168185, 168186, 168187, 168188, 168189, 168190, 168191) c6( ) c7(T, hd, AS, bP, ae, ad); #c1(CBX2) c2(NP_005180) c3(chromobox protein homolog 2 isoform 1) c4(153665) c5(153666, 153667, 153668, 153669, 153670, 153671, 153672) c6( ) c7(aBS, KZ, T, av, UL); #c1(CBX2) c2(NP_116036) c3(chromobox protein homolog 2 isoform 2) c4(225273) c5(225274, 225275, 225276, 225277, 225278, 225279) c6( ) c7(aBS, KZ, T, av, UL); #c1(CBX3) c2(NP_009207) c3(chromobox protein homolog 3) c4(171505, 171506) c5(171507, 171508, 171509, 171510, 171511, 171512, 171513, 171514, 171515, 171516, 171517, 171518, 171519, 171520, 171521, 171522, 171523, 171524) c6( ) c7(sb, ae, T, wu, eD, n, rO, wm, bz, iu, ad); #c1(CBX3) c2(XP_005249669) c3(chromobox protein homolog 3 isoform X2) c4(269771) c5(269772, 269773, 269774, 269775, 269776) c6( ) c7(sb, ae, T, wu, eD, n, rO, wm, bz, iu, ad); #c1(CBX4) c2(NP_003646) c3(E3 SUMO-protein ligase CBX4) c4(139895) c5(139896, 139897, 139898, 139899, 139900, 139901, 139902, 139903, 139904, 139905, 139906, 139907, 139908) c6( ) c7(n, ae, B, ad, Ub); #c1(CBX4) c2(XP_011523701) c3(E3 SUMO-protein ligase CBX4 isoform X1) c4(318175) c5(318176) c6( ) c7(n, ae, B, ad, Ub); #c1(CBX5) c2(NP_001120793) c3(chromobox protein homolog 5) c4(44760, 44761) c5(44762, 44763, 44764, 44765, 44766, 44767, 44768, 44769, 44770, 44771, 44772, 44773, 44774, 44775, 44776, 44777, 44778, 44779, 44780, 44781, 44782) c6( ) c7(ck, ae, T, hd, iU, wu, aW, aBT, eD, dV, aT, bO, rO, ci, wm, bz, ad); #c1(CBX6) c2(NP_001290423) c3(chromobox protein homolog 6 isoform 2) c4(115396) c5(115397, 115398, 115399, 115400, 115401, 115402, 115403) c6( ) c7(dl, as); #c1(CBX6) c2(NP_055107) c3(chromobox protein homolog 6 isoform 1) c4(178987) c5( ) c6( ) c7(dL, as); #c1(CBX6) c2(XP_011528326) c3(chromobox protein homolog 6 isoform X1) c4( ) c5(323382, 323383, 323384, 323385, 323386, 323387, 323388) c6( ) c7(dL, as); #c1(CBX7) c2(NP_783640) c3(chromobox protein homolog 7) c4(251698, 251699) c5(25/700, 251701, 251702, 251703, 25/704, 25/705, 25/706, 25/707, 251708, 251709, 251710, 251711, 251712) c6( ) c7(T, fM, jh, aA, bB, fd, ci, eJ, q, ad, hd, aE, ae, d, s, qt, Y, r, aF, g, oU, nB, bE, eD); #c1(CBX7) c2(XP_005261470) c3(chromobox protein homolog 7 isoform X7) c4(277361) c5(277362, 277363, 277364, 277365, 277366, 277367) c6( ) c7(T, fM, jh, aA, bB, fd, ci, eJ, q, ad, hd, aE, ae, d, s, qt, Y, r, aF, g, oU, nB, bE, eD); #c1(CBX8) c2(NP_065700) c3(chromobox protein homolog 8) c4(205004) c5(205005, 205006, 205007, 205008, 205009, 205010) c6( ) c7(aE, Bj, aW, aw, aT, dL); #c1(CBY1) c2(NP_001002880) c3 (protein chibby homolog 1 isoform b) c4( ) c5(19724, 19725, 19726, 19727) c6( ) c7(fR, sQ, aBU, awP, r, uo, ut, lQ, q); #c1(CBY1) c2(NP_056188) c3(protein chibby homolog 1 isoform a) c4(186192, 186193, 186194) c5(186195, 186196, 186197, 186198, 186199, 186200, 186201, 186202, 186203, 186204, 186205, 186206, 186207, 186208, 186209, 186210, 186211, 186212) c6( ) c7(fR, sQ, aBU, awP, r, uo, ut, lQ, q); #c1(CC2D1A) c2(NP_060191) c3(coiled-coil and C2 domain-containing protein (A) c4(194698, 194699) c5(194700, 194701, 194702, 194703, 194704, 194705, 194706) c6( ) c7(W, aBV, bO, h, K, nA, bz); #c1(CC2D1A) c2(XP_005260030) c3(coiled-coil and C2 domain-containing protein (A isoform X2) c4(276402, 276403) c5(276404, 276405, 276406, 276407, 276408, 276409, 276410, 276411, 276412, 276413, 276414, 276415) c6( ) c7(W, aBV, bO, h, K, nA, bz); #c1(CC2D1A) c2(XP_005260032) c3(coiled-coil and CZ domain-containing protein IA isoform X4) c4( ) c5(276416, 276417, 276418, 276419, 276420) c6( ) c7(W, aBV, bO, h, K, nA, bz); #c1(CC2DIB) c2(XP_011539232) c3(coiled-coil and C2 domain-containing protein 1B isoform X5) c4( ) c5(337334, 337335, 337336, 337337, 337338, 337339) c6( ) c7(h); #c1(CC2D2A) c2(NP_001073991) c3(coiled-coil and C2 domain-containing protein 2A isoform a) c4(36811, 36812) c5(36813, 36814, 36815, 36816, 36817, 36818, 36819, 36820, 36821, 36822, 36823, 36824, 36825, 36826, 36827, 36828, 36829, 36830, 36831, 36832, 36833, 36834, 36835, 36836, 36837, 36838, 36839, 36840, 36841, 36842, 36843, 36844, 36845, 36846) c6( ) c7(aBW, ul, ago, Gp, hl, vm, aBZ, Nj, aBY, nA, aBX, nC); #c1(CC2D2A) c2(NP_065836) c3(coiled-coil and C2 domain-containing protein 2A isoform b) c4(205777) c5(205778, 205779, 205780, 205781, 205782, 205783, 205784, 205785, 205786, 205787, 205788, 205789, 205790, 205791, 205792, 205793) c6( ) c7(aBW, ul, ago, Gp, hl, vm, aBZ, Nj, aBY, nA, aBX, nC); #c1(CC2D2A) c2(XP_011512174) c3(coiled-coil and CZ domain-containing protein 2A isoform X5) c4(302739) c5( ) c6( ) c7(aBW, ul, ago, Gp, hl, vm, aBZ, Nj, aBY, nA, aBX, nC); #c1(CC2D2A) c2(XP_011512176) c3(coiled-coil and C2 domain-containing protein 2A isoform X2) c4(302740) c5(302741, 302742, 302743, 302744, 302745, 302746) c6( ) c7(aBW, ul, ago, Gp, hl, vm, aBZ, Nj, aBY, nA, aBX, nC); #c1(CCAR1) c2(NP_001269889) c3(cell division cycle and apoptosis regulator protein 1 isoform b) c4(103042, 103043) c5(103044, 103045, 103046, 103047, 103048, 103049, 103050, 103051, 103052, 103053, 103054, 103055, 103056, 103057, 103058, 103059, 103060, 103061, 103062, 103063, 103064, 103065, 103066, 103067, 103068, 103069, 103070, 103071, 103072, 103073) c6( ) c7(T, ok, dA, bE, jh, aT, bB, he); #c1(CCAR1) c2(NP_060707) c3(cell division cycle and apoptosis regulator protein 1 isoform a) c4(197313, 197314, 197315) c5( ) c6( ) c7(T, ok, dA, bE, jh, aT, bB, he); #c1(CCAR2) c2(NP_066997) c3(cell cycle and apoptosis regulator protein 2) c4(208081) c5(208082, 208083, 208084, 208085, 208086, 208087, 208088, 208089, 208090, 208091) c6( ) c7(x, T, id, Qu, aW, eL, fc, jh, aT, n, dL, v, ci, K, cP, kp, aF, ae, iu, ad); #c1(CCBE1) c2(NP_597716) c3(collagen and calcium-binding EGF domain-containing protein 1 precursor) c4(235089, 235090) c5(235091, 235092, 235093, 235094, 235095, 235096, 235097, 235098, 235099, 235100, 235101, 235102) c6( ) c7(aW, aCa, aT, m, nA, hJ, t, fq, y); #c1(CCBL1) c2(NP_001116144) c3(kynurenine-oxoglutarate transaminase 1 isoform b) c4(43654, 43655) c5(43656, 43657, 43658, 43659, 43660, 43661, 43662, 43663, 43664, 43665, 43666, 43667, 43668, 43669, 43670) c6( ) c7(es, fO, ci); #c1(CCBL1) c2(NP_001274319) c3(kynurenine-oxoglutarate transaminase 1 isoform c) c4(106230) c5(106231, 106232, 106233, 106234, 106235, 106236) c6( ) c7(es, fO, ci); #c1(CCBL1) c2(XP_0067/7374) c3(kynurenine-oxoglutarate transaminase 1 isoform X4) c4(291038, 291039) c5(291040) c6( ) c7(es, fl, ci); #c1 (CCBL1) c2(XP_011517467) c3(kynurenine-oxoglutarate transaminase 1 isoform X2) c4(311186) c5(311187, 311188, 311189) c6( ) c7(es, fO, ci); #c1(CCBL1) c2(XP_011517468) c3(kynurenine-oxoglutarate transaminase 1 isoform X3) c4( ) c5(311190, 311191, 311192, 311193, 311194, 311195, 311196, 311197, 311198, 311199) c6( ) c7(es, fO, ci); #c1(CCBL1) c2(XP_011517472) c3(kynurenine-oxoglutarate transaminase 1 isoform X1) c4( ) c5(311200, 311201, 311202, 311203, 311204) c6( ) c7(es, fO, ci); #c1(CCDC101) c2(NP_612423) c3(SAGA-associated factor 29 homolog) c4(236435) c5(236436, 236437, 236438, 236439, 236440, 236441) c6( ) c7(dy, K); #c1(CCDC102B) c2(NP_001087198) c3(coiled-coil domain-containing protein 102B) c4(38257) c5(38258, 38259, 38260, 38261, 38262, 38263, 38264, 38265, 38266, 38267) c6( ) c7(fq); #c1(CCDC102B) c2(XP_011524476) c3(coiled-coil domain-containing protein 1028 isoform X4) c4(319065) c5(319066, 319067, 319068, 319069, 319070, 319071, 319072) c6( ) c7(fq); #c1(CCDC103) c2(NP_001245326) c3(coiled-coil domain-containing protein 103 isoform 2) c4( ) c5(93123) c6( ) c7(Pf, aCb, MG); #c1(CCDC103) c2(NP_998772) c3(coiled-coil domain-containing protein 103 isoform 1) c4(265922, 265923) c5(265924, 265925, 265926, 265927, 265928, 265929, 265930, 265931, 265932, 265933, 265934, 265935, 265936, 265937, 265938, 265939) c6( ) c7(Pf, aCb, MG); #c1(CCDC105) c2(NP_775753) c3(coiled-coil domain-containing protein 105) c4(250168) c5(250169, 250170, 250171, 250172, 250173, 250174) c6( ) c7(oe); #c1(CCDC108) c2(NP_001265224) c3(coiled-coil domain-containing protein 108 isoform 3) c4(97957, 97958) c5(97959, 97960, 97961, 97962, 97963, 97964) c6( ) c7(cd); #c1(CCDC108) c2(NP_001265225) c3(coiled-coil domain-containing protein 108 isoform 4) c4( ) c5(97965, 97966, 97967, 97968, 97969, 97970) c6( ) c7(cd); #c1(CCDC108) c2(NP_689602) c3(coiled-coil domain-containing protein 108 isoform 2) c4(243210) c5(243211, 243212, 243213, 243214, 243215, 243216, 243217, 243218, 243219, 243220) c6( ) c7(cd); #c1(CCDC108) c2(NP_919278) c3(coiled-coil domain-containing protein 108 isoform 1) c4( ) c5(258449, 258450, 258451, 258452, 258453, 258454, 258455, 258456, 258457, 258458, 258459) c6( ) c7(cd); #c1(CCDC108) c2(XP_011509211) c3(coiled-coil domain-containing protein 108 isoform X2) c4( ) c5(299866) c6( ) c7(cd); #c1(CCDC108) c2(XP_011509212) c3(coiled-coil domain-containing protein 108 isoform X3) c4(299867) c5(299868, 299869, 299870) c6( ) c7(cd); #c1(CCDC114) c2(NP_653/78) c3(coiled-coil domain-containing protein 114) c4(238965) c5(238966, 238967, 238968, 238969, 238970) c6( ) c7(Pf, aCc, MG, bD); #c1(CCDC114) c2(XP_005259472) c3(coiled-coil domain-containing protein 114 isoform X2) c4( ) c5(276112) c6( ) c7(Pf, aCc, MG, bD); #c1(CCDC115) c2(NP_115733) c3(coiled-coil domain-containing protein 115) c4(223738) c5(223739, 223740, 223741, 223742, 223743, 223744) c6( ) c7(cm, n, CQ); #c1 (CCDC115) c2(XP_005263882) c3(coiled-coil domain-containing protein (15 isoform X1) c4(278569) c5(278570, 278571, 278572, 278573, 278574, 278575) c6( ) c7(cm, n, CQ); #c1(CCDC121) c2(NP_001136155) c3(coiled-coil domain-containing protein (21 isoform 2) c4(54443) c5(54444, 54445, 54446, 54447, 54448, 54449, 54450) c6( ) c7(om); #c1(CCDC121) c2(NP_078860) c3(coiled-coil domain-containing protein (21 isoform 3) c4(215826) c5(215827, 215828, 215829, 215830, 215831) c6( ) c7(om); #c1(CCDC122) c2(NP_659411) c3(coiled-coil domain-containing protein (22) c4(239771) c5(239772, 239773, 239774, 239775, 239776, 239777) c6( ) c7(acD, fq, AW, acC); #c1(CCDC122) c2(XP_011533245) c3(coiled-coil domain-containing protein 122 isoform X1) c4(329378, 329379, 329380, 329381, 329382, 329383, 329384) c5(329385, 329386, 329387, 329388, 329389, 329390, 329391, 329392, 329393, 329394, 329395, 329396, 329397, 329398, 329399, 329400, 329401, 329402, 329403, 329404, 329405, 329406, 329407, 329408, 329409, 329410, 329411, 329412) c6( ) c7(acD, fq, AW, acC); #c1(CCDC129) c2(NP_001244896) c3(coiled-coil domain-containing protein 129 isoform 1) c4(92293, 92294) c5(92295, 92296, 92297, 92298, 92299, 92300, 92301, 92302, 92303) c6( ) c7(Ea); #c1(CCDC129) c2(NP_001244897) c3(coiled-coil domain-containing protein (29 isoform 3) c4(92304, 92305, 92306, 92307) c5(92308, 92309, 92310, 92311, 92312, 92313, 92314) c6( ) c7(Ea); #c1(CCDC129) c2(NP_919276) c3(coiled-coil domain-containing protein 129 isoform 2) c4(258414) c5(258415, 258416, 258417, 258418, 258419, 258420, 258421, 258422, 258423, 258424, 258425, 258426, 258427, 258428, 258429, 258430, 258431, 258432, 258433, 258434, 258435, 258436, 258437, 258438, 258439, 258440) c6( ) c7(Ea); #c1(CCDC129) c2(XP_011513518) c3(coiled-coil domain-containing protein 129 isoform X2) c4( ) c5(305803) c6( ) c7(Ea); #c1(CCDC130) c2(NP_110445) c3(coiled-coil domain-containing protein 130) c4(220397, 220398) c5(220399, 220400, 220401, 220402, 220403, 220404) c6( ) c7(zx); #c1(CCDC130) c2(XP_011526628) c3(coiled-coil domain-containing protein 130 isoform X2) c4( ) c5(320928, 320929, 320930, 320931, 320932, 320933) c6( ) c7(zx); #c1(CCDC134) c2(NP_001291726) c3(coiled-coil domain-containing protein 134 isoform 2 precursor) c4(116419) c5(116420, 116421, 116422, 116423, 116424, 116425, 116426) c6(116427) c7(aq, af); #c1(CCDC134) c2(XP_005261805) c3(coiled-coil domain-containing protein 134 isoform X1) c4(277616) c5(277617, 277618, 277619, 277620, 277621, 277622, 277623, 277624) c6(277625, 277626) c7(aq, af); #c1(CCDC136) c2(NP_001188301) c3(coiled-coil domain-containing protein 136 isoform 2) c4(79030) c5(79031, 79032, 79033, 79034, 79035, 79036, 79037, 79038, 79039, 79040, 79041, 79042, 79043, 79044, 79045, 79046, 79047, 79048, 79049, 79050, 79051, 79052, 79053, 79054, 79055) c6( ) c7(ci); #c1(CCDC136) c2(NP_073579) c3(coiled-coil domain-containing protein 136 isoform 1) c4(212945, 212946, 212947) c5(212948, 212949, 212950, 212951, 212952, 212953) c6( ) c7(ci); #c1(CCDC136) c2(XP_005250591) c3(coiled-coil domain-containing protein 136 isoform X5) c4(270430) c5(270431, 270432, 270433, 270434, 270435, 270436) c6( ) c7(ci); #c1(CCDC136) c2(XP_005250595) c3(coiled-coil domain-containing protein 136 isoform X9) c4(270437) c5(270438, 270439, 270440, 270441, 270442, 270443) c6( ) c7(ci); #c1(CCDC136) c2(XP_006716154) c3(coiled-coil domain-containing protein 136 isoform X13) c4(290099) c5(290100, 290101, 290102, 290103, 290104, 290105) c6( ) c7(ci); #c1(CCDC136) c2(XP_011514786) c3(coiled-coil domain-containing protein 136 isoform X2) c4(307438) c5( ) c6( ) c7(ci); #c1(CCDC140) c2(NP_694583) c3(coiled-coil domain-containing protein 140) c4(245316, 245317) c5(245318, 245319, 245320, 245321, 245322, 245323, 245324, 245325, 245326, 245327, 245328, 245329) c6( ) c7(bM); #c1(CCDC141) c2(NP_775919) c3(coiled-coil domain-containing protein 141) c4(250683, 250684, 250685) c5(250686, 250687, 250688, 250689, 250690, 250691, 250692, 250693, 250694, 250695, 250696, 250697, 250698, 250699, 250700, 250701, 250702, 250703) c6( ) c7(cz); #c1(CCDC141) c2(XP_011509296) c3(coiled-coil domain-containing protein 141 isoform X4) c4( ) c5(299906) c6( ) c7(cz); #c1(CCDC148) c2(NP_001288613) c3(coiled-coil domain-containing protein 148 isoform 3) c4( ) c5(113839, 113840, 113841, 113842, 113843, 113844) c6( ) c7(bN, bA); #c1(CCDC148) c2(NP_620158) c3(coiled-coil domain-containing protein 148 isoform 1) c4(237388) c5(237389, 237390, 237391, 237392, 237393, 237394, 237395, 237396, 237397, 237398, 237399, 237400) c6( ) c7(bN, bA); #c1(CCDC148) c2(XP_011508940) c3(coiled-coil domain-containing protein 148 isoform X1) c4(299502) c5( ) c6( ) c7(bN, bA); #c1(CCDC148) c2(XP_011508941) c3(coiled-coil domain-containing protein 148 isoform X3) c4(299503) c5( ) c6( ) c7(bN, bA); #c1(CCDC151) c2(NP_001289382) c3(coiled-coil domain-containing protein 151 isoform 2) c4(114164) c5(114165, 114166, 114167, 114168, 114169, 114/70) c6( ) c7(Pf, MG, bD); #c1(CCDC151) c2(NP_659482) c3(coiled-coil domain-containing protein 151 isoform 1) c4(240151, 240152) c5(240153, 240154, 240155, 240156, 240157, 240158, 240159, 240160, 240161, 240162, 240163, 240164) c6( ) c7(Pf, MG, bD); #c1(CCDC167) c2(NP_612502) c3(coiled-coil domain-containing protein 167) c4(236648, 236649, 236650) c5(236651, 236652, 236653, 236654, 236655, 236656, 236657, 236658, 236659, 236660, 236661, 236662, 236663, 236664, 236665, 236666, 236667, 236668) c6( ) c7(aw, cd, af); #c1(CCDC169-SOHLH2) c2(NP_001185839) c3(CCDC169-SOHLH2 protein) c4(76570) c5(76571, 76572, 76573, 76574, 76575, 76576, 76577, 76578, 76579) c6( ) c7(il, t, T, zZ, m); #c1(CCDC170) c2(NP_079335) c3(coiled-coil domain-containing protein (70) c4(217755, 217756) c5(217757, 217758, 217759, 217760, 217761, 217762, 217763, 217764, 217765, 217766, 217767, 217768) c6( ) c7(aT); #c1(CCDC171) c2(NP_775821) c3(coiled-coil domain-containing protein (71) c4(250411, 250412) c5(250413, 250414, 250415, 250416, 250417, 250418, 250419, 250420, 250421, 250422, 250423, 250424) c6( ) c7(dY); #c1(CCDC171) c2(XP_011516092) c3(coiled-coil domain-containing protein 171 isoform X3) c4(309297) c5(309298, 309299, 309300, 309301, 309302, 309303) c6( ) c7(dY); #c1(CCDC171) c2(XP_011516094) c3(coiled-coil domain-containing protein (71 isoform X5) c4(309304) c5( ) c6( ) c7(dY); #c1(CCDC171) c2(XP_011516095) c3(coiled-coil domain-containing protein 171 isoform X6) c4(309305, 309306) c5(309307) c6( ) c7(dY); #c1(CCDC171) c2(XP_011516096) c3(coiled-coil domain-containing protein (71 isoform X7) c4(309308) c5( ) c6( ) c7(dY); #c1(CCDC(71) c2(XP_011516097) c3(coiled-coil domain-containing protein 171 isoform X8) c4( ) c5(309309, 309310, 309311, 309312, 309313, 309314) c6( ) c7(dY); #c1(CCDC(71) c2(XP_011516098) c3(coiled-coil domain-containing protein 171 isoform X9) c4( ) c5(309315, 309316, 309317, 309318, 309319, 309320, 309321, 309322, 309323, 309324, 309325, 309326, 309327, 309328, 309329, 309330, 309331) c6( ) c7(dY); #c1(CCDC175) c2(NP_001157871) c3(coiled-coil domain-containing protein (75) c4(62836, 62837) c5(62838, 62839, 62840, 62841, 62842, 62843, 62844, 62845, 62846, 62847, 62848, 62849) c6( ) c7(af); #c1(CCDC176) c2(NP_079333) c3(basal body-orientation factor 1) c4(217748) c5(217749, 217750, 217751, 217752, 217753, 217754) c6( ) c7(kl); #c1(CCDC176) c2(XP_011535474) c3(basal body-orientation factor 1 isoform X3) c4(332541) c5(332542, 332543, 332544, 332545, 332546, 332547) c6( ) c7(kl); #c1(CCDC178) c2(NP_001098998) c3(coiled-coil domain-containing protein (78 isoform 1) c4(41576, 41577) c5(41578, 41579, 41580, 41581, 41582, 41583, 41584, 41585, 41586, 41587) c6( ) c7(ad); #c1(CCDC178) c2(NP_945346) c3(coiled-coil domain-containing protein 178 isoform 2) c4(260850) c5(260851, 260852, 260853, 260854, 260855, 260856) c6( ) c7(ad); #c1(CCDC178) c2(XP_011524253) c3(coiled-coil domain-containing protein 178 isoform X7) c4(318742) c5(318743, 318744, 318745, 318746, 318747, 318748) c6( ) c7(ad); #c1(CCDC(78) c2(XP_011524257) c3(coiled-coil domain-containing protein 178 isoform X10) c4( ) c5(318749, 318750, 318751, 318752, 318753, 318754) c6( ) c7(ad); #c1(CCDC180) c2(NP_065944) c3(coiled-coil domain-containing protein 180) c4(206360, 206361, 206362, 206363, 206364) c5(206365, 206366, 206367, 206368, 206369, 206370, 206371, 206372, 206373, 206374, 206375, 206376, 206377, 206378, 206379, 206380, 206381, 206382, 206383, 206384, 206385, 206386, 206387, 206388, 206389, 206390, 206391, 206392, 206393, 206394, 206395, 206396, 206397, 206398, 206399) c6( ) c7(hH); #c1(CCDC181) c2(NP_067002) c3(coiled-coil domain-containing protein 181 isoform a) c4(208239) c5(208240, 208241, 208242, 208243, 208244, 208245, 208246, 208247, 208248, 208249, 208250, 208251, 208252, 208253, 208254, 208255) c6( ) c7(n, cz); #c1(CCDC181) c2(XP_011508129) c3(coiled-coil domain-containing protein 181 isoform X1) c4(298616, 298617) c5(298618, 298619, 298620, 298621, 298622, 298623) c6( ) c7(n, cz); #c1(CCDC185) c2(NP_689823) c3(coiled-coil domain-containing protein 185) c4(244030) c5(244031, 244032, 244033, 244034, 244035, 244036, 244037, 244038, 244039, 244040, 244041, 244042, 244043) c6( ) c7(j); #c1(CCDC22) c2(NP_054727) c3(coiled-coil domain-containing protein 22) c4(177367, 177368) c5(177369, 177370, 177371, 177372, 177373, 177374, 177375, 177376, 177377, 177378, 177379, 177380) c6( ) c7(af); #c1(CCDC28A) c2(NP_056254) c3(coiled-coil domain-containing protein 28A) c4(186748, 186749) c5(186750, 186751, 186752, 186753, 186754, 186755, 186756, 186757, 186758, 186759, 186760, 186761, 186762, 186763) c6( ) c7(iY, hF, dz, iT, et); #c1(CCDC3) c2(NP_001269587) c3(coiled-coil domain-containing protein 3 isoform 2) c4(102076) c5(102077, 102078, 102079, 102080, 102081, 102082, 102083, 102084) c6(102085, 102086) c7(v); #c1(CCDC3) c2(NP_113643) c3(coiled-coil domain-containing protein 3 isoform 1 precursor) c4(221900) c5(221901, 221902, 221903, 221904, 221905, 221906, 221907, 221908) c6(221909, 221910) c7(v); #c1(CCDC39) c2(NP_852091) c3(coiled-coil domain-containing protein 39) c4(254813, 254814) c5(254815, 254816, 254817, 254818, 254819, 254820, 254821, 254822, 254823, 254824, 254825, 254826) c6( ) c7(Pf, aCd, MG); #c1(CCDC40) c2(NP_001230271) c3(coiled-coil domain-containing protein 40 isoform 2) c4(86177) c5(86178, 86179, 86180, 86181, 86182, 86183) c6( ) c7(Pf, aCe, MG); #c1(CCDC40) c2(NP_060420) c3(coiled-coil domain-containing protein 40 isoform 1) c4(195883, 195884) c5(195885, 195886, 195887, 195888, 195889, 195890) c6( ) c7(Pf, aCe, MG); #c1(CCDC40) c2(XP_005257549) c3(coiled-coil domain-containing protein 40 isoform X4) c4(275249) c5(275250, 275251, 275252, 275253, 275254, 275255, 275256, 275257, 275258, 275259, 275260, 275261) c6( ) c7(Pf, aCe, MG); #c1(CCDC42) c2(NP_001151733) c3(coiled-coil domain-containing protein 42A isoform 2) c4(60121) c5(60122, 60123, 60124, 60125, 60126, 60127) c6( ) c7(hH); #c1(CCDC42) c2(NP_653282) c3(coiled-coil domain-containing protein 42A isoform 1) c4(239497) c5(239498, 239499, 239500, 239501, 239502, 239503) c6( ) c7(hH); #c1(CCDC42B) c2(NP_001138344) c3(coiled-coil domain-containing protein 428) c4(56192) c5(56193, 56194, 56195, 56196, 56197, 56198) c6( ) c7(F, B, hl); #c1(CCDC50) c2(NP_777568) c3(coiled-coil domain-containing protein 50 short isoform) c4(251150) c5(251151, 251152, 251153, 251154, 251155, 251156) c6( ) c7(aCf, cj); #c1(CCDC50) c2(NP_848018) c3(coiled-coil domain-containing protein 50 long isoform) c4(253699) c5(253700, 253701, 253702, 253703, 253704, 253705) c6( ) c7(aCf, cj); #c1(CCDC54) c2(NP_115989) c3(coiled-coil domain-containing protein 54) c4(225119) c5(225120, 225121, 225122, 225123, 225124, 225125) c6( ) c7(Kc, T, eM, if, ff, ht, bu, hL, fd, m, en, hu, qN, t, g, c1); #c1(CCDC6) c2(NP_005427) c3(coiled-coil domain-containing protein 6) c4(156040) c5(156041, 156042, 156043, 156044, 156045, 156046) c6( ) c7(T, aCh, AS, wu, LX, Dd, n, hz, ci, aon, mF, xY, aCi, Vp, iu, hd, xy, azj, db, nv, Qg, aT, dz, Fx, K, wm, pi, jf, VC, d, aCg, vT, aW, r, bz, aAW, qJ, sS, nM, nB, hk, YH, aeH, AN, LT); #c1(CCDC60) c2(NP_848594) c3(coiled-coil domain-containing protein 60) c4(253935) c5(253936, 253937, 253938, 253939, 253940, 253941) c6( ) c7(bL, j); #c1(CCDC62) c2(NP_958843) c3(coiled-coil domain-containing protein 62) c4(262265, 262266) c5(262267, 262268, 262269, 262270, 262271, 262272) c6( ) c7(aiZ, Gt, ajx, an, U, al, bu, ae, ad); #c1(CCDC62) c2(XP_006719707) c3(coiled-coil domain-containing protein 62 isoform X3) c4(292897) c5(292898, 292899, 292900, 292901, 292902, 292903) c6( ) c7(aiZ, Gt, ajx, an, U, aU, bu, ae, ad); #c1(CCDC62) c2(XP_011537160) c3(coiled-coil domain-containing protein 62 isoform X5) c4( ) c5(334920, 334921, 334922, 334923, 334924, 334925) c6( ) c7(aiZ, Gt, ajx, an, U, aU, bu, ae, ad); #c1(CCDC63) c2(XP_011536303) c3(coiled-coil domain-containing protein 63 isoform X1) c4(333641) c5(333642, 333643, 333644, 333645, 333646, 333647) c6( ) c7(Y); #c1(CCDC65) c2(NP_149115) c3(coiled-coil domain-containing protein 65 isoform 1) c4(227245, 227246) c5(227247, 227248, 227249, 227250, 227251, 227252, 227253, 227254, 227255, 227256, 227257, 227258) c6( ) c7(Pf, aCj, MG); #c1(CCDC66) c2(NP_001012524) c3(coiled-coil domain-containing protein 66 isoform 2) c4(25027) c5(25028, 25029, 25030, 25031, 25032) c6( ) c7(nC, aCk); #c1(CCDC66) c2(NP_001135419) c3(coiled-coil domain-containing protein 66 isoform 1) c4(52876, 52877) c5(52878, 52879, 52880, 52881, 52882, 52883, 52884, 52885, 52886, 52887, 52888, 52889, 52890) c6( ) c7(nC, aCk); #c1(CCDC66) c2(XP_005265138) c3(coiled-coil domain-containing protein 66 isoform X1) c4( ) c5(279441) c6( ) c7(nC, aCk); #c1(CCDC66) c2(XP_005265144) c3(coiled-coil domain-containing protein 66 isoform X11) c4( ) c5(279442, 279443, 279444, 279445, 279446, 279447, 279448) c6( ) c7(nC, aCk); #c1(CCDC66) c2(XP_006713178) c3(coiled-coil domain-containing protein 66 isoform X3) c4(287050) c5( ) c6( ) c7(nC, aCk); #c1(CCDC67) c2(NP_857596) c3(deuterosome protein 1) c4(255265) c5(255266, 255267, 255268, 255269, 255270, 255271, 255272, 255273, 255274, 255275) c6( ) c7(bM, aF, aA); #c1(CCDC67) c2(XP_011540937) c3(deuterosome protein 1 isoform X6) c4(339464) c5( ) c6( ) c7(bM, aF, aA); #c1(CCDC67) c2(XP_011540939) c3(deuterosome protein 1 isoform X8) c4( ) c5(339465, 339466, 339467, 339468, 339469, 339470) c6( ) c7(bM, aF, aA); #c1(CCDC68) c2(NP_001137301) c3(coiled-coil domain-containing protein 68) c4(55387) c5(55388, 55389, 55390, 55391, 55392, 55393, 55394, 55395, 55396, 55397, 55398, 55399, 55400) c6( ) c7(j, Eg); #c1(CCDC78) c2(NP_001026907) c3(coiled-coil domain-containing protein 78) c4(29969) c5(29970, 29971, 29972, 29973, 29974, 29975) c6( ) c7(aCl, oj, Ad); #c1(CCDC78) c2(XP_005255163) c3(coiled-coil domain-containing protein 78 isoform X19) c4( ) c5(273570) c6( ) c7(aCl, oj, Ad); #c1(CCDC78) c2(XP_006720906) c3(coiled-coil domain-containing protein 78 isoform X16) c4(293779) c5( ) c6( ) c7(aCl, oj, Ad); #c1(CCDC78) c2(XP_011520673) c3(coiled-coil domain-containing protein 78 isoform X18) c4( ) c5(315603, 315604, 315605, 315606, 315607) c6( ) c7(aCl, oj, Ad); #c1(CCDC8) c2(NP_114429) c3(coiled-coil domain-containing protein 8) c4(222733, 222734) c5(222735, 222736, 222737, 222738, 222739, 222740, 222741, 222742, 222743, 222744, 222745, 222746) c6( ) c7(T, dD, hl, kP, asl, aW, Y, ea, aT, aCm, nw, aj, eq, fr, pl, aCo, ik, aCn, de); #c1(CCDC80) c2(NP_955805) c3(coiled-coil domain-containing protein 80 precursor) c4(261980, 261981) c5(261982, 261983, 261984, 261985, 261986, 261987, 261988, 261989, 261990, 261991, 261992, 261993, 261994, 261995, 261996, 261997, 261998, 261999, 262000, 262001, 262002, 262003) c6(262004, 262005, 262006, 262007) c7(T, hd, db, nB, eF, eH, eE, ad); #c1(CCDC83) c2(NP_001273088) c3(coiled-coil domain-containing protein 83 isoform 2) c4(104313) c5(104314, 104315, 104316, 104317, 104318, 104319) c6( ) c7(bB, bE, bu); #c1(CCDC83) c2(XP_011543143) c3(coiled-coil domain-containing protein 83 isoform X1) c4(341957) c5(341958, 341959, 341960, 341961, 341962, 341963) c6( ) c7(bB, bE, bu); #c1(CCDC83) c2(XP_011543145) c3(coiled-coil domain-containing protein 83 isoform X3) c4(341964) c5(341965, 341966, 341967, 341968, 341969, 341970) c6( ) c7(bB, bE, bu); #c1(CCDC85A) c2(NP_001073902) c3(coiled-coil domain-containing protein 85A) c4(36412) c5(36413, 36414, 36415, 36416, 36417, 36418) c6( ) c7(v, j, pN); #c1(CCDC85A) c2(XP_005264182) c3(coiled-coil domain-containing protein 85A isoform X1) c4( ) c5(278699) c6( ) c7(v, j, pN); #c1(CCDC86) c2(NP_077003) c3(coiled-coil domain-containing protein 86) c4(214680, 214681, 214682) c5(214683, 214684, 214685, 214686, 214687, 214688, 214689, 214690, 214691, 214692, 214693, 214694, 214695, 214696, 214697, 214698, 214699, 214700) c6( ) c7(ac); #c1(CCDC88A) c2(NP_001129069) c3(girdin isoform 1) c4(50723) c5(50724, 50725, 50726, 50727, 50728, 50729) c6( ) c7(T, ac, c, aE, bE, aW, aT, r, pR, bB, K, q); #c1(CCDC88A) c2(NP_001241872) c3(girdin isoform 3) c4(89122) c5(89123, 89124, 89125, 89126, 89127, 89128, 89129, 89130, 89131, 89132, 89133, 89134, 89135, 89136, 89137, 89138, 89139, 89140, 89141) c6(89142, 89143, 89144, 89145, 89146, 89147, 89148) c7(T, ac, c, aE, bE, aW, aT, r, pR, bB, K, q); #c1(CCDC88A) c2(XP_005264478) c3(girdin isoform X5) c4(278955) c5(278956, 278957, 278958, 278959, 278960, 278961) c6( ) c7(T, ac, c, aE, bE, aW, aT, r, pR, bB, K, q); #c1(CCDC88A) c2(XP_011531268) c3 (girdin isoform X4) c4(327241) c5(327242, 327243, 327244, 327245, 327246, 327247) c6( ) c7(T, ac, c, aE, bE, aW, aT, r, pR, bB, K, q); #c1(CCDC88A) c2(XP_011531269) c3(girdin isoform X1) c4(327248, 327249) c5(327250, 327251, 327252, 327253, 327254, 327255, 327256, 327257, 327258, 327259, 327260, 327261) c6( ) c7(T, ac, c, aE, bE, aW, aT, r, pR, bB, K, q); #c1(CCDC88C) c2(NP_001073883) c3(protein Daple) c4(36287, 36288) c5(36289, 36290, 36291, 36292, 36293, 36294) c6( ) c7(Nq, ul, Nr, aW, aT, dz, Xm, fq); #c1(CCDC88C) c2(XP_005267748) c3(protein Daple isoform X2) c4(281229) c5(281230, 281231, 281232, 281233, 281234, 281235) c6( ) c7(Nq, ul, Nr, aW, aT, dz, Xm, fq); #c1(CCDC88C) c2(XP_011535098) c3(protein Daple isoform X1) c4(331835) c5(331836, 331837, 331838, 331839, 331840, 331841, 331842, 331843, 331844, 331845, 331846, 331847) c6( ) c7(Nq, ul, Nr, aW, aT, dz, Xm, fq); #c1(CCDC91) c2(NP_060788) c3(coiled-coil domain-containing protein 91) c4(197816, 197817, 197818, 197819, 197820) c5(197821, 197822, 197823, 197824, 197825, 197826, 197827, 197828, 197829, 197830, 197831, 197832, 197833, 197834, 197835, 197836, 197837, 197838, 197839, 197840, 197841, 197842, 197843, 197844, 197845, 197846, 197847, 197848, 197849, 197850, 197851, 197852, 197853, 197854, 197855, 197856, 197857, 197858) c6( ) c7(IB, aCp, abV, Be); #c1(CCDC91) c2(XP_011519034) c3(coiled-coil domain-containing protein 91 isoform X4) c4(313501) c5(313502, 313503, 313504, 313505, 313506, 313507) c6( ) c7(IB, aCp, abV, Be); #c1(CCDC94) c2(NP_060544) c3(coiled-coil domain-containing protein 94) c4(196485) c5(196486, 196487, 196488, 196489, 196490, 196491) c6( ) c7(bt); #c1(CCDC97) c2(NP_443080) c3(coiled-coil domain-containing protein 97) c4(229308) c5(229309, 229310, 229311, 229312, 229313, 229314) c6( ) c7(dl); #c1(CCHCR1) c2(NP_001099033) c3(coiled-coil alpha-helical rod protein 1 isoform 2) c4(41672) c5(41673, 41674, 41675, 41676, 41677, 41678, 41679, 41680, 41681, 41682) c6( ) c7(bL, bM, T, yk, e, NT, cz, aj, v, j, C, J, ad, wD, afV, K, lQ, hu, hH, xa, aCr, Nr, aCq, ff, cr, eZ, po, pD, en, iV, Nq, vK, sS, Jr, z, hk, Gs, ei); #c1(CCHCR1) c2(NP_001099034) c3(coiled-coil alpha-helical rod protein 1 isoform 1) c4(41683) c5(41684, 41685, 41686, 41687, 41688, 41689, 41690, 41691, 41692, 41693, 41694, 41695, 41696, 41697, 41698, 41699, 41700, 41701, 41702, 41703, 41704, 41705, 41706, 41707, 41708, 41709, 4/710, 4/711, 41712, 41713, 41714) c6( ) c7(bL, bM, T, yk, e, NT, cz, aj, v, j, C, J, ad, wD, afV, K, lQ, hu, hH, xa, aCr, Nr, aCq, ff, cr, eZ, po, pD, en, iV, Nq, vK, sS, Jr, z, hk, Gs, ei); #c1(CCHCR1) c2(XP_006725551) c3(coiled-coil alpha-helical rod protein 1 isoform X9) c4(296884, 296885, 296886, 296887, 296888, 296889) c5(296890, 296891, 296892, 296893, 296894, 296895, 296896, 296897, 296898, 296899, 296900, 296901, 296902, 296903, 296904, 296905, 296906, 296907, 296908, 296909, 296910, 296911) c6( ) c7(bL, bM, T, yk, e, NT, cz, aj, v, j, C, J, ad, wD, afV, K, lQ, hu, hH, xa, aCr, Nr, aCq, ff, cr, eZ, po, pD, en, iV, Nq, vK, sS, Jr, z, hk, Gs, ei); #c1 (CCHCR1) c2(XP_011513004) c3(coiled-coil alpha-helical rod protein 1 isoform X8) c4( ) c5(304453, 304454) c6( ) c7(bL, bM, T, yk, e, NT, cz, aj, v, j, C, J, ad, wD, afV, K, lQ, hu, hH, xa, aCr, Nr, aCq, ff, cr, eZ, po, pD, en, iV, Nq, vK, sS, Jr, z, hk, Gs, ei); #c1(CCHCR1) c2(XP_011513005) c3(coiled-coil alpha-helical rod protein 1 isoform X9) c4(304455, 304456, 304457, 304458, 304459, 304460, 304461, 304462, 304463, 304464, 304465) c5(304466, 304467, 304468, 304469, 304470, 304471, 304472, 304473, 304474, 304475, 304476, 304477, 304478, 304479, 304480, 304481) c6( ) c7(bL, bM, T, yk, e, NT, cz, aj, v, j, C, J, ad, wD, afV, K, lQ, hu, hH, xa, aCr, Nr, aCq, ff, cr, eZ, po, pD, en, iV, Nq, vK, sS, Jr, z, hk, Gs, ei); #c1(CCHCR1) c2(XP_011513007) c3(coiled-coil alpha-helical rod protein 1 isoform X10) c4(304482, 304483, 304484) c5(304485, 304486, 304487, 304488, 304489, 304490, 304491, 304492, 304493, 304494, 304495, 304496, 304497, 304498, 304499) c6( ) c7(bL, bM, T, yk, e, NT, cz, aj, v, j, C, J, ad, wD, afV, K, lQ, hu, hH, xa, aCr, Nr, aCq, ff, cr, eZ, po, pD, en, iV, Nq, vK, sS, Jr, z, hk, Gs, ei); #c1(CCHCR1) c2(XP_011513008) c3(coiled-coil alpha-helical rod protein 1 isoform X2) c4( ) c5(304500, 304501, 304502, 304503, 304504, 304505, 304506, 304507, 304508, 304509, 304510, 304511, 304512, 304513, 304514, 304515, 304516, 304517, 304518, 304519, 304520, 304521, 304522, 304523, 304524, 304525, 304526, 304527, 304528, 304529, 304530, 304531, 304532, 304533, 304534, 304535, 304536, 304537, 304538, 304539, 304540, 304541, 304542, 304543, 304544, 304545, 304546, 304547, 304548, 304549, 304550, 304551, 304552, 304553, 304554, 304555, 304556, 304557, 304558, 304559, 304560, 304561, 304562, 304563, 304564, 304565, 304566, 304567, 304568, 304569, 304570, 304571, 304572, 304573, 304574, 304575, 304576, 304577, 304578, 304579, 304580, 304581, 304582, 304583, 304584, 304585, 304586, 304587, 304588, 304589, 304590, 304591, 304592, 304593, 304594, 304595, 304596, 304597, 304598, 304599, 304600, 304601, 304602, 304603, 304604, 304605, 304606, 304607, 304608, 304609, 304610, 304611, 304612, 304613, 304614, 304615, 304616, 304617, 304618, 304619, 304620, 304621, 304622, 304623, 304624, 304625, 304626) c6( ) c7(bL, bM, T, yk, e, NT, cz, aj, v, j, C, J, ad, wD, afV, K, lQ, hu, hH, xa, aCr, Nr, aCq, ff, cr, eZ, po, pD, en, iV, Nq, vK, sS, Jr, z, hk, Gs, ei); #c1(CCHCR1) c2(XP_011545560) c3(coiled-coil alpha-helical rod protein 1 isoform X10) c4(344329, 344330) c5(344331, 344332, 344333, 344334, 344335, 344336, 344337, 344338) c6( ) c7(bL, bM, T, yk, e, NT, cz, aj, v, j, C, J, ad, wD, afV, K, lQ, hu, hH, xa, aCr, Nr, aCq, ff, cr, eZ, po, pD, en, iV, Nq, vK, sS, Jr, z, hk, Gs, ei); #c1(CCHCR1) c2(XP_011545561) c3(coiled-coil alpha-helical rod protein 1 isoform X2) c4( ) c5(344339, 344340, 344341, 344342, 344343, 344344, 344345, 344346, 344347, 344348, 344349, 344350, 344351, 344352, 344353, 344354) c6( ) c7(bL, bM, T, yk, e, NT, cz, aj, v, j, C, J, ad, wD, afV, K, lQ, hu, hH, xa, aCr, Nr, aCq, ff, cr, eZ, po, pD, en, iV, Nq, vK, sS, Jr, z, hk, Gs, ei); #c1(CCHCR1) c2(XP_011545562) c3(coiled-coil alpha-helical rod protein 1 isoform X1) c4( ) c5(344355, 344356, 344357, 344358, 344359, 344360, 344361, 344362, 344363) c6( ) c7(bL, bM, T, yk, e, NT, cz, aj, v, j, C, J, ad, wD, afV, K, lQ, hu, hH, xa, aCr, Nr, aCq, ff, cr, eZ, po, pD, en, iV, Nq, vK, sS, Jr, z, hk, Gs, ei); #c1(CCHCR1) c2(XP_011546361) c3(coiled-coil alpha-helical rod protein 1 isoform X9) c4(345076, 345077, 345078, 345079, 345080, 345081, 345082) c5(345083, 345084, 345085, 345086, 345087, 345088, 345089, 345090, 345091, 345092, 345093, 345094, 345095, 345096, 345097, 345098, 345099, 345100, 345101, 345102, 345103, 345104) c6( ) c7(bL, bM, T, yk, e, NT, cz, aj, v, j, C, J, ad, wD, afV, K, lQ, hu, hH, xa, aCr, Nr, aCq, ff, cr, eZ, po, pD, en, iV, Nq, vK, sS, Jr, z, hk, Gs, ei); #c1(CCHCR1) c2(XP_011546363) c3(coiled-coil alpha-helical rod protein 1 isoform X10) c4(345105) c5(345106, 345107, 345108, 345109, 345110, 345111, 345112, 345113) c6( ) c7(bL, bM, T, yk, e, NT, cz, aj, v, j, C, J, ad, wD, afV, K, lQ, hu, hH, xa, aCr, Nr, aCq, ff, cr, eZ, po, pD, en, iV, Nq, vK, sS, Jr, z, hk, Gs, ei); #c1 (CCHCR1) c2(XP_011546547) c3(coiled-coil alpha-helical rod protein 1 isoform X6) c4(345384) c5(345385, 345386, 345387, 345388, 345389, 345390, 345391, 345392, 345393, 345394) c6( ) c7(bL, bM, T, yk, e, NT, cz, aj, v, j, C, J, ad, wD, afV, K, lQ, hu, hH, xa, aCr, Nr, aCq, ff, cr, eZ, po, pD, en, iV, Nq, vK, sS, Jr, z, hk, Gs, ei); #c1(CCHCR1) c2(XP_011546548) c3(coiled-coil alpha-helical rod protein 1 isoform X7) c4(345395, 345396) c5(345397, 345398, 345399, 345400, 345401, 345402, 345403, 345404, 345405, 345406, 345407, 345408, 345409, 345410, 345411, 345412, 345413, 345414, 345415, 345416, 345417, 345418, 345419, 345420, 345421, 345422, 345423, 345424, 345425, 345426, 345427, 345428, 345429, 345430, 345431, 345432, 345433) c6( ) c7(bL, bM, T, yk, e, NT, cz, aj, v, j, C, J, ad, wD, afV, K, IQ, hu, hH, xa, aCr, Nr, aCq, ff, cr, eZ, po, pD, en, iV, Nq, vK, sS, Jr, z, hk, Gs, ei); #c1(CCK) c2(NP_000720) c3(cholecystokinin preproprotein) c4(14861) c5(14862, 14863, 14864, 14865, 14866, 14867, 14868, 14869, 14870, 14871, 14872, 14873, 14874, 14875) c6( ) c7(bL, cZ, T, aC, dA, ya, jh, aA, ti, aj, bS, v, ci, iG, ww, Wb, q, ad, eA, eJ, jF, bv, ip, l, ft, hd, u, hW, aE, av, mj, pX, io, Sb, Ou, gl, bK, dd, bq, he, hB, ae, dl, dx, na, an, li, fb, kl, lx, tb, s, Gv, vj, aCt, AQ, hl, aCs, r, nY, aF, hp, g, as, LV, FX, fq, t, cm, Jh, cv, hq, ar, iq, eM, dB, bE, Gs, gX, sG, m, ra, cE, Ex, On); #c1 (CCKAR) c2(NP_000721) c3(cholecystokinin receptor type A) c4(14876) c5(14877, 14878, 14879, 14880, 14881, 14882) c6( ) c7(bL, aC, bO, aA, aj, v, ww, qi, eA, J, ip, l, aE, u, hW, iX, aql, Sb, dL, bK, an, na, tb, vj, aF, g, FX, hq, ZT, On); #c1(CCKBR) c2(NP_795344) c3(gastrin/cholecystokinin type B receptor) c4(252416) c5(252417, 252418, 252419, 252420, 252421, 252422) c6( ) c7(bL, T, aC, fM, aCw, On, aD, aA, bB, ww, ci, q, ad, eA, eJ, ip, av, ft, hd, aE, hW, nv, ji, aCv, bq, an, fb, ae, B, tb, s, JI, Y, aCt, az, r, cu, aF, g, hq, ck, aCu, nB, bE, gX, X, Ex, qc); #c1(CCKBR) c2(XP_005253267) c3(gastrin/cholecystokinin type B receptor isoform X1) c4(272352) c5(272353, 272354, 272355, 272356, 272357, 272358) c6( ) c7(bL, T, aC, fM, aCw, On, aD, aA, bB, ww, ci, q, ad, eA, eJ, ip, av, ft, hd, aE, hW, nv, ji, aCv, bq, an, fb, ae, B, tb, s, JI, Y, aCt, az, r, cu, aF, g, hq, ck, aCu, nB, bE, gX, X, Ex, qc); #c1 (CCL1) c2(NP_002972) c3(C-C motif chemokine 1 precursor) c4(134087) c5(134088, 134089, 134090, 134091, 134092, 134093) c6( ) c7(bM, T, Dw, iW, V, dP, dY, dV, aT, h, cU, kH, Ds, hu, uD, je, ff, r, yq, en, adz, iM, hc, at, va, lC, it); #c1(CCL11) c2(NP_002977) c3(eotaxin precursor) c4(134141) c5(134142, 134143, 134144, 134145, 134146, 134147) c6( ) c7(bL, bM, T, kh, dD, aCA, Dw, akc, eR, fG, aw, aj, v, j, ci, C, pM, eq, fr, V, J, dP, fS, M, h, lG, Ds, JK, ji, eu, Nr, A, zH, sp, aCx, mQ, aCB, fe, akY, po, iV, eE, t, cM, DF, Nq, TA, kT, cT, ac, aCz, Jp, JM, at, aCy, fD, ei, ra); #c1 (CCL13) c2(NP_005399) c3(C-C motif chemokine 13 precursor) c4(155709, 155710, 155711) c5(155712, 155713, 155714, 155715, 155716, 155717) c6( ) c7(Nq, aCC, bM, Nr, Dw, z, hc, U, cj, bt, h, Ds, va, Xm, fD, eE, t, V, y); #c1(CCL14) c2(NP_116738) c3(C-C motif chemokine 14 isoform 2 precursor) c4(226315) c5(226316, 226317, 226318, 226319, 226320, 226321, 226322, 226323, 226324, 226325, 226326) c6( ) c7(dP, bM, gk, Dw, B, eJ, eR, aW, al, eZ, aT, fe, Ds, fl, hF, V, bz, mC, hX); #c1(CCL14) c2(NP_116739) c3(C-C motif chemokine 14 isoform 1 precursor) c4(226327) c5(226328, 226329, 226330, 226331, 226332, 226333) c6( ) c7(dP, bM, gk, Dw, B, eJ, eR, aW, al, eZ, aT, fe, Ds, fL, hF, V, bz, mC, hX); #c1(CCL15) c2(NP_116741) c3(C-C motif chemokine 15 preproprotein) c4(226334, 226335) c5(226336, 226337, 226338, 226339, 226340, 226341, 226342, 226343, 226344, 226345, 226346, 226347, 226348, 226349, 226350, 226351, 226352, 226353, 226354, 226355, 226356, 226357, 226358, 226359, 226360, 226361) c6(226362) c7(bM, Dw, eR, fe, Ds, B); #c1(CCL16) c2(XP_005258077) c3(C-C motif chemokine 16 isoform X1) c4(275480) c5(275481, 275482, 275483, 275484, 275485, 275486) c6( ) c7(bM, Dw, aaQ, aE, iU, sw, Y, aW, aT, hl, aT, dN, aq, Ds, ci, aAo, adz, hE, B, ad); #c1(CCL17) c2(NP_002978) c3(C-C motif chemokine 17 precursor) c4(134148) c5(134149, 134150, 134151, 134152, 134153) c6( ) c7(bM, T, dD, fM, Dw, afA, ZI, ea, fG, jx, eX, jh, aA, eq, Qp, V, aR, o, dP, aCF, lg, od, ds, kH, cU, eV, Ds, lQ, amY, aCG, Nr, je, Y, fJ, md, hl, cr, eZ, dN, aCE, po, ff, en, bu, eE, Nq, LB, cT, ck, iM, eU, xT, vZ, xO, z, ME, fD, aCD, i, lf, bt, ao); #c1(CCL17) c2(XP_011521558) c3(C-C motif chemokine 17 isoform X1) c4( ) c5(316292) c6( ) c7(bM, T, dD, fM, Dw, afA, ZI, ea, fG, jx, ex, jh, aA, eq, Qp, V, aR, o, dP, aCF, lg, od, ds, kH, cU, eV, Ds, lQ, amY, aCG, Nr, je, Y, fJ, md, hl, cr, eZ, dN, aCE, po, ff, en, bu, eE, Nq, LB, cT, ck, iM, eU, xT, vZ, xO, z, ME, fD, aCD, i, lf, bt, ao); #c1 (CCL18) c2(NP_002979) c3(C-C motif chemokine 18 precursor) c4(134154) c5(134155, 134156, 134157, 134158, 134159, 134160, 134161) c6(134162) c7(bM, T, dD, Dw, akc, afA, ea, fG, j, eq, hX, x, av, F, dY, lg, aT, h, Ds, vB, lQ, bA, aa, je, aW, aCH, eE, cm, pb, LB, ck, ou, vU, iM, iQ, z, cG, hs, aCl, XO, k, aCD, y); #c1 (CCL19) c2(NP_006265) c3(C-C motif chemokine 19 precursor) c4(163422, 163423) c5(163424, 163425, 163426, 163427, 163428, 163429, 163430, 163431, 163432, 163433, 163434, 163435) c6( ) c7(bM, Dw, dD, fR, ea, cl, v, j, eq, V, cd, o, aCJ, kH, aT, h, Ds, vB, KU, ul, je, iU, aW, lh, lb, mQ, fJ, cr, bz, Wt, xT, Bv, iM, Dy, eU, xO, cj, eo, fD, iu); #c1(CCL2) c2(NP_002973) c3(C-C motif chemokine 2 precursor) c4(134094) c5(134095, 134096, 134097, 134098, 134099, 134100) c6( ) c7(T, ea, bO, fd, ci, aY, aDI, bv, xy, aE, fm, rw, dV, Ds, aCL, aCU, LG, hH, aCN, cp, aa, li, ly, az, uY, vs, PQ, zC, GR, ba, nB, aCT, lp, wE, cj, k, lS, aDi, de, vE, cQ, aj, qW, cd, zo, TB, aDo, lG, ey, mJ, mR, anP, sp, EU, fQ, fe, aDu, dB, aoB, aA, cz, bX, q, aCX, agD, Oi, cy, aCR, rz, dl, eu, bf, fO, bg, lo, cl, pb, tR, Vn, OW, ac, iQ, ig, CP, DW, MB, bt, On, aCA, wi, iW, KO, dG, fu, ad, hX, oW, lQ, DI, cH, cW, ff, tv, iV, bz, DF, anY, ME, lf, lC, iu, lD, bM, aiR, if, e, bB, cL, bh, Tz, ip, x, Na, eG, up, fc, hl, aDn, kt, as, ir, eE, eF, cm, vA, vy, tV, aCy, hh, aCS, dD, zt, n, aDc, aCZ, uE, md, Mo, aDa, DU, A, fJ, lb, hQ, bP, dC, cA, hL, at, pg, ut, eo, aDb, fL, cB, B, NO, dA, mr, Lx, aDq, v, V, WY, Nw, ld, aDr, aDt, aDh, ae, CB, jf, iZ, t, vk, ck, wl, zu, vi, hl, wV, eq, qi, BY, ds, aCJ, JK, uD, nJ, sj, d, wc, NH, dN, aCK, Hv, D, gk, Hs, agl, aCP, y, aC, fR, atj, al, CH, lR, wa, AW, Bc, bW, M, K, GO, ajK, dF, eL, Us, aCM, mQ, g, uC, Vm, aDs, U, uo, Hb, m, NK, QO, sQ, aw, BH, apt, aCQ, pU, JW, ft, gq, cU, Ne, By, Jn, wb, aDp, Ha, ar, kT, W, mg, gg, Pr, it, L, iH, fG, jx, hz, j, vp, eA, J, o, hb, nX, aCV, Nc, bq, Y, Pg, r, ls, tu, en, iK, c, hc, dh, azk, tM, Da, cS, dy, fD, amL, sY, id, ah, SY, iG, dP, fU, sN, vB, ji, adx, eH, cr, hp, Fq, Bu, qa, Bv, nb, Lj, cP, ei, G, aR, dY, aCW, aDk, YX, aCO, bU, oi, H, dl, hm, amN, aW, HA, eM, nw, My, ez, fr, qN, ai, eW, ke, cc, jL, zf, h, jy, Nf, dL, db, wC, OV, aDe, aq, kp, mo, op, lx, aCz, bm, uz, bD, bL, Dw, eR, sM, tc, C, cF, aCY, acj, mP, Vr, aT, bk, an, af, oV, aDf, aaE, aDm, f, sS, vZ, AH, wq, aDj, Kd, ha, vr, Fb, jE, pz, mC, KL, LH, F, ib, sO, Os, jo, iL, yF, ul, qt, yx, eZ, yy, aF, aDd, LB, es, axs, bE, uR, aDg, aS, X, us); #c1 (CCL20) c2(NP_001123518) c3(C-C motif chemokine 20 isoform 2 precursor) c4(47294) c5(47295, 47296, 47297, 47298, 47299, 47300) c6( ) c7(bM, T, id, fR, aD, oe, eX, fd, cl, aj, ci, V, q, o, J, ad, eA, hd, aE, lg, aT, hD, eV, ji, K, lQ, bq, pk, ae, bc, eu, DA, A, B, je, xR, aW, az, hl, mQ, r, cr, aq, fe, vs, yq, iV, BE, bz, ck, fT, aDv, iM, zr, nB, AM, at, uz, wR, fD, Pk, X, y); #c1(CCL20) c2(NP_004582) c3(C-C motif chemokine 20 isoform 1 precursor) c4(147880) c5(147881, 147882, 147883, 147884, 147885, 147886) c6( ) c7(bM, T, id, fR, aD, oe, eX, fd, cl, aj, ci, V, q, o, J, ad, eA, hd, aE, lg, aT, hD, eV, ji, K, lQ, bq, pk, ae, bc, eu, DA, A, B, je, xR, aW, az, hl, mQ, r, cr, aq, fe, vs, yq, iV, eE, bz, ck, fT, aDv, iM, zr, nB, AM, at, uz, wR, fD, Pk, X, y); #c1(CCL21) c2(NP_002980) c3(C-C motif chemokine 21 precursor) c4(134163, 134164) c5(134165, 134166, 134167, 134168, 134169, 134170, 134/71, 134/72, 134173, 134174, 134175) c6( ) c7(oO, bM, T, dD, fR, JC, ea, e, fN, cz, bB, jh, cl, j, eq, V, Uy, o, q, cd, dP, ai, eW, hd, F, wD, KH, aT, h, azm, ey, hu, KU, hm, hM, A, je, s, iU, xR, aW, Y, lh, lb, ev, cr, r, iV, t, cm, bz, pb, Wt, aDw, LB, Bv, iM, Dy, eM, nB, bE, eU, ajd, tV, cj, aDx, aS, m, yH, y, B); #c1(CCL21) c2(XP_011516306) c3(C-C motif chemokine 21 isoform X1) c4( ) c5(309522) c6( ) c7(oO, bM, T, dD, fR, JC, ea, e, fN, cz, bB, jh, cL, j, eq, V, Uy, o, q, cd, dP, ai, eW, hd, F, wD, kH, aT, h, azm, ey, hu, KU, hm, hM, A, je, s, iU, xR, aW, Y, lh, lb, ev, cr, r, iV, t, cm, bz, pb, Wt, aDw, LB, Bv, iM, Dy, eM, nB, bE, eU, ajd, tV, cj, aDx, aS, m, yH, y, B); #c1(CCL22) c2(NP_002981) c3(C-C motif chemokine 22 precursor) c4(134/76) c5(134/77, 134178, 134179, 134180, 134181, 134182) c6( ) c7(bM, T, Dw, ZI, fG, aw, jx, eX, jh, aA, cz, eq, Qp, V, aR, wD, av, lg, kH, cU, aT, Ds, dL, h, eV, amY, eL, uQ, je, anP, aW, hl, cr, af, eE, asE, iP, LB, cT, fT, iM, ac, hk, hc, cj, ME, fD, aCD, i, y, bt); #c1(CCL23) c2(NP_005055) c3(C-C motif chemokine 23 isoform CKbeta8-1 precursor) c4(152482) c5(152483, 152484, 152485, 152486, 152487, 152488) c6( ) c7(bM, Dw, h, Ds, eE, y); #c1(CCL23) c2(NP_665905) c3(C-C motif chemokine 23 isoform CKbeta8 precursor) c4(241970) c5(241971, 241972, 241973, 241974, 241975, 241976) c6( ) c7(bM, Dw, h, Ds, eE, y); #c1(CCL24) c2(XP_011514762) c3(C-C motif chemokine 24 isoform X1) c4(307406) c5(307407, 307408, 307409, 307410, 307411, 307412, 307413, 307414, 307415, 307416) c6( ) c7(Nq, bM, Dw, Nr, tk, fR, aDy, z, sp, fS, mQ, JM, dN, h, Ds, Xm, fD, iV, eE); #c1(CCL25) c2(NP_001188288) c3(C-C motif chemokine 25 isoform 2 precursor) c4(79010) c5(79011, 79012, 79013, 79014, 79015, 79016, 79017, 79018, 79019, 79020, 79021, 79022) c6( ) c7(bM, xN, dD, Dw, ea, v, eq, ad, dP, aT, h, Ds, ae, xQ, aW, Y, vj, t, Dy, z, cj, m, y); #c1(CCL25) c2(XP_011526479) c3(C-C motif chemokine 25 isoform X1) c4(320776, 320777) c5(320778, 320779, 320780, 320781, 320782, 320783) c6( ) c7(bM, xN, dD, Dw, ea, v, eq, ad, dP, aT, h, Ds, ae, xQ, aW, Y, vj, t, Dy, z, cj, m, y); #c1(CCL26) c2(NP_006063) c3(C-C motif chemokine 26 precursor) c4(161568) c5(161569, 161570, 161571, 161572, 161573, 161574, 161575, 161576, 161577, 161578, 161579, 161580, 161581, 161582) c6(161583) c7(bM, Dw, hl, akc, fR, aoj, oz, cE, h, Ds, Nr, Du, dN, akY, po, fe, iV, eE, fq, DF, Nq, Ta, z, fD, y, it, hv, Tc); #c1(CCL27) c2(NP_006655) c3(C-C motif chemokine 27 precursor) c4(166946) c5(166947, 166948, 166949, 166950, 166951, 166952) c6( ) c7(bM, T, Dw, xS, fd, bB, TI, aT, h, Ds, lQ, nJ, bc, xP, avg, je, xR, vT, HY, aW, TH, fJ, eH, cr, dN, aq, eE, eF, pb, xT, iM, TJ, xO, TG, bE, au, cj, rx, i, y, in, B); #c1(CCL28) c2(NP_001288802) c3(C-C motif chemokine 28 isoform a precursor) c4(114083, 114084, 114085) c5(114086, 114087, 114088, 114089, 114090, 114091, 114092, 114093, 114094, 114095, 114096, 114097, 114098, 114099, 114100, 114101, 114102, 114/03, 114104, 114105, 114106, 114107, 114108) c6( ) c7(bM, T, Dw, aC, fR, LX, iB, cz, v, ci, afb, aT, kH, h, Ds, lQ, aCt, CM, bw, Ji, cj, dy, bt, ra, LT); #c1(CCL28) c2(NP_001288804) c3(C-C motif chemokine 28 isoform b) c4( ) c5(114109, 114110, 114/11, 114/12, 114113, 114114) c6(114115) c7(bM, T, Dw, aC, fR, LX, iB, cz, v, ci, afb, aT, kH, h, Ds, lQ, aCt, CM, bw, Ji, cj, dy, bt, ra, LT); #c1(CCL3) c2(NP_002974) c3(C-C motif chemokine 3 precursor) c4(134101) c5(134102, 134103, 134104, 134105, 134106, 134107) c6( ) c7(bM, T, fR, ea, fd, cL, AW, dV, K, mQ, zC, g, eE, oy, cj, uo, Hb, hh, dD, sQ, aik, n, uE, ai, zw, h, lG, jy, aDp, mJ, Nr, aq, po, dB, ut, cB, B, DD, fG, Lx, j, V, eA, o, rS, aT, cy, an, af, eu, kl, bf, iU, axW, aCH, fO, en, ck, iQ, iM, ac, ig, aDA, wl, hc, AH, wR, fD, bt, ao, Ke, jh, eq, aDz, ds, aCJ, vB, lQ, yF, ul, d, je, ff, gE, iV, ek, Nq, LB, axs, aDB, aS, y, X); #c1(CCL3L3) c2(NP_001001437) c3(C-C motif chemokine 3-like 1 precursor) c4(18444, 18445, 18446, 18447, 18448) c5(18449, 18450, 18451, 18452, 18453, 18454, 18455, 18456, 18457, 18458, 18459, 18460, 18461, 18462, 18463, 18464, 18465, 18466, 18467, 18468, 18469, 18470, 18471, 18472, 18473, 18474, 18475, 18476, 18477, 18478, 18479, 18480, 18481, 18482, 18483, 18484, 18485, 18486) c6(18487, 18488, 18489, 18490) c7(bM, A, F, iU, axW, jx, lf, oW, dV, h, cr, gA, eZ, IQ, Pk, bt, y, hX); #c1 (CCL4) c2(NP_002975) c3(C-C motif chemokine 4 precursor) c4(134108, 134109, 134110, 134/11, 134112, 134113, 134114) c5(134115, 134116, 134117, 134118, 134119, 134120, 134121, 134122, 134123, 134124, 134125, 134126) c6( ) c7(bL, bM, T, dD, Dw, Ke, ea, fG, Lx, al, hE, jx, fd, jh, gA, j, ci, C, bX, eq, pz, amW, KL, LH, zn, hX, F, xy, ds, dV, kH, aT, h, Ds, K, aDC, lQ, ajK, ZN, af, eu, yF, d, je, s, aW, aCH, mQ, hl, cr, aq, iV, f, KM, dr, ck, gk, iM, ac, iQ, ig, hc, lf, cj, wR, fD, be, ei, Pk, bt, B, y); #c1(CCL4LI) c2(NP_996890) c3(C-C motif chemokine 4-like precursor) c4(264826) c5(264827, 264828, 264829, 264830, 264831, 264832, 264833, 264834, 264835, 264836, 264837, 264838, 264839, 264840, 264841, 264842, 264843, 264844, 264845, 264846, 264847, 264848, 264849, 264850, 264851, 264852, 264853) c6(264854, 264855, 264856) c7(bL, T, dD, Ke, ea, al, jx, jh, BH, gA, v, eq, C, bX, kL, LH, zn, hX, F, xy, ds, dV, kH, aT, h, K, aDC, ZN, yF, d, je, s, aW, aCH, hl, cr, aq, iV, f, KM, dr, ck, gk, iM, ac, lf, cj, wR, ME, be, ei, hE, bt, cB, B, y); #c1(CCL4L2) c2(NP_001278397) c3(C-C motif chemokine 4-like isoform 1 precursor) c4( ) c5(109667) c6( ) c7(bL, T, dD, Ke, ea, al, jx, jh, gA, eq, C, bX, KL, LH, zn, hX, F, xy, ds, dV, kH, aT, h, K, aDC, ZN, yF, d, je, s, aW, aCH, hl, cr, aq, iV, f, KM, dr, ck, gk, iM, ac, lf, cj, wR, be, ei, hE, bt, B, y); #c1(CCL4L2) c2(NP_001278400) c3(C-C motif chemokine 4-like isoform 3 precursor) c4(109668) c5(109669, 109670, 109671, 109672, 109673, 109674, 109675, 109676) c6(109677, 109678) c7(bL, T, dD, Ke, ea, al, jx, jh, gA, eq, C, bX, KL, LH, zn, hX, F, xy, ds, dV, kH, aT, h, K, aDC, ZN, yF, d, je, s, aW, aCH, hl, cr, aq, iV, f, KM, dr, ck, gk, iM, ac, lf, cj, wR, be, ei, hE, bt, B, y); #c1(CCL4L2) c2(NP_001278402) c3(C-C motif chemokine 4-like isoform 5 precursor) c4(109679) c5(109680, 109681, 109682, 109683, 109684, 109685) c6( ) c7(bL, T, dD, Ke, ea, al, jx, jh, gA, eq, C, bX, KL, LH, zn, hX, F, xy, ds, dV, kH, aT, h, K, aDC, ZN, yF, d, je, s, aW, aCH, hl, cr, aq, iV, f, KM, dr, ck, gk, iM, ac, lf, cj, wR, be, ei, hE, bt, B, y); #c1(CCL4L2) c2(NP_001278403) c3(C-C motif chemokine 4-like isoform 6 precursor) c4(109686, 109687) c5(109688, 109689, 109690, 109691, 109692, 109693, 109694, 109695, 109696, 109697, 109698, 109699, 109700) c6(109701) c7(bL, T, dD, Ke, ea, al, jx, jh, gA, eq, C, bX, kL, LH, zn, hX, F, xy, ds, dV, kH, aT, h, K, aDC, ZN, yF, d, je, s, aW, aCH, hl, cr, aq, iV, f, KM, dr, ck, gk, iM, ac, lf, cj, wR, be, ei, hE, bt, B, y); #c1(CCL5) c2(NP_001265665) c3(C-C motif chemokine 5 isoform 2 precursor) c4( ) c5(99188, 99189) c6( ) c7(oO, bM, T, lD, FR, ea, atj, e, al, fd, bB, cL, bh, aY, fw, sD, amW, aR, wa, xy, aE, rw, bW, dV, Ds, K, da, hH, bc, aa, ly, eG, aiV, aW, az, hl, mQ, aDn, hN, aB, zC, Ku, g, as, ve, axp, uC, eE, y, fT, aDD, aiM, dW, Dt, Dv, aDM, fy, aDi, qR, iu, dD, My, aw, n, aDI, aj, fr, pM, qW, ai, eW, aDF, zw, ec, wG, ju, cU, lG, h, dl, ey, eV, aDp, Nr, A, sp, fe, aq, po, cM, lx, ar, lf, JM, at, eo, fl, aDE, gg, Pr, B, bL, Dw, dA, eR, aDK, fG, jx, eX, YN, aA, rf, hz, v, j, ek, C, bX, aaV, aDO, V, q, J, o, eA, aCY, apJ, Nw, qp, afQ, aT, Qi, jj, an, ae, af, hM, Uf, s, xR, oV, Y, fO, apk, r, lo, aDL, en, bu, pb, Cv, cT, OW, ac, iQ, ig, hc, aDG, AH, MB, wq, mf, dy, Gg, fD, Da, bt, Kd, aCA, Ke, qb, aD, dM, df, iW, jh, vr, dG, eq, jE, Fb, ad, wD, hX, dP, fU, av, F, ds, aCJ, oW, Os, hD, vB, jo, Od, adx, yF, sj, ul, d, cH, ff, wc, cr, eZ, aF, hp, uF, iV, bz, aDH, DF, aDw, Nq, D, gk, aDN, es, zr, axs, bE, atM, eU, z, X, aDJ, ME, agl, ei, ol, lC, gq, hv); #c1(CCL5) c2(NP_002976) c3(C-C motif chemokine 5 isoform 1 precursor) c4(134127, 134128) c5(134129, 134130, 134131, 134132, 134133, 134134, 134135, 134136, 134137, 134138) c6(134139, 134140) c7(oO, bM, T, lD, fR, ea, atj, e, al, fd, bB, cL, bh, aY, fw, sD, amW, aR, wa, xy, aE, rw, bW, dV, Ds, K, dQ, hH, bc, aa, ly, eG, aiV, aW, az, hl, mQ, aDn, hN, aB, zC, Ku, g, as, ve, axp, uC, eE, y, fT, aDD, aiM, dW, Dt, Dv, aDM, fy, aDi, qR, iu, dD, My, aw, n, aDI, aj, fr, pM, qW, ai, eW, aDF, zw, ec, wG, ju, cU, lG, h, dL, ey, eV, aDp, Nr, A, sp, fe, aq, po, cM, lx, ar, lf, JM, at, eo, fl, aDE, gg, Pr, B, bL, Dw, dA, eR, aDK, fG, jx, eX, YN, aA, rf, hz, v, j, ek, C, bX, aaV, aDO, V, q, J, o, eA, aCY, apJ, Nw, qp, afQ, aT, Oi, j, an, ae, af, hM, Uf, s, xR, oV, Y, fO, apk, r, lo, aDL, en, bu, pb, Cv, cT, OW, ac, iQ, ig, hc, aDG, AH, MB, wq, mf, dy, Gg, fD, Da, bt, Kd, aCA, Ke, qb, aD, dM, df, iW, jh, vr, dG, eq, jE, Fb, ad, wD, hX, dP, fU, av, F, ds, aCJ, oW, Os, hD, vB, jo, Od, adx, yF, sj, ul, d, cH, ff, wc, cr, eZ, aF, hp, uF, iV, bz, aDH, DF, aDw, Nq, D, gk, aDN, es, zr, axs, bE, atM, eU, z, X, aDJ, ME, agl, ei, oQ, lC, gq, hv); #c1(CCL7) c2(NP_006264) c3(C-C motif chemokine 7 precursor) c4(163404, 163405) c5(163406, 163407, 163408, 163409, 163410, 163411, 163412, 163413, 163414, 163415, 163416, 163417, 163418, 163419, 163420, 163421) c6( ) c7(bM, T, NK, Dw, wi, bB, fr, V, ai, eA, wa, aE, h, Ds, jj, uD, rz, ff, mQ, fQ, fe, en, aDP, hJ, t, cm, pb, LB, bE, hc, Ny, ajd, va, fD, y, X); #c1(CCL8) c2(NP_005614) c3(C-C motif chemokine 8 precursor) c4(157949, 157950) c5(157951, 157952, 157953, 157954, 157955, 157956, 157957, 157958, 157959, 157960, 157961, 157962, 157963) c6( ) c7(bM, Dw, fR, sQ, iW, ex, CH, ol, v, V, q, ai, eA, ady, F, WY, ds, h, eV, Ds, lQ, af, CB, ff, mQ, eZ, r, yy, aaE, en, iV, eE, t, aDQ, eM, Jp, AH, uo, ut, aDR, m, fD, hh, y); #c1(CCM2) c2(NP_001025006) c3(malcavernin isoform 1) c4(29180, 29181) c5(29182, 29183, 29184, 29185, 29186, 29187, 29188, 29189, 29190, 29191, 29192) c6( ) c7(bL, jf, y, d, nO, dB, Ot, aDT, cj, aT, cl, v, cm, o, ae, aDS, ad); #c1(CCM2) c2(NP_001161406) c3(malcavernin isoform 3) c4(65757) c5(65758, 65759, 65760, 65761, 65762, 65763) c6( ) c7(bL, jf, y, d, nO, dB, Ot, aDT, cj, aT, cl, v, cm, o, ae, aDS, ad); #c1(CCM2) c2(NP_001161407) c3(malcavernin isoform 4) c4(65764) c5(65765, 65766, 65767, 65768, 65769, 65770, 65771, 65772, 65773, 65774, 65775, 65776) c6( ) c7(bL, jf, y, d, nO, dB, Ot, aDT, cj, aT, cl, v, cm, o, ae, aDS, ad); #c1(CCM2) c2(NP_113631) c3(malcavernin isoform 2) c4(221824) c5(221825, 221826, 221827, 221828, 221829, 221830) c6( ) c7(bL, jf, y, d, nO, dB, Ot, aDT, cj, aT, cL, v, cm, o, ae, aDS, ad); #c1 (CCM2) c2(XP_011513864) c3(malcavernin isoform X2) c4(306280) c5( ) c6( ) c7(bL, jf, y, d, nO, dB, Ot, aDT, cj, aT, cl, v, cm, o, ae, aDS, ad); #c1 (CCNA1) c2(NP_001104515) c3(cyclin-A1 isoform b) c4(42153) c5(42154, 42155, 42156, 42157, 42158, 42159, 42160) c6( ) c7(T, gU, if, al, n, oY, fr, ci, qN, Ul, q, ad, x, pp, aoA, aT, vH, h, dL, ae, eL, iU, aW, Lf, ff, r, en, bu, t, iK, bz, Zj, ck, gk, gn, k, bt, B); #c1(CCNA1) c2(NP_003905) c3(cyclin-A1 isoform a) c4(142043) c5(142044, 142045, 142046, 142047, 142048, 142049, 142050) c6( ) c7(T, gU, if, al, n, oY, fr, ci, qN, Ul, q, ad, x, pp, aoA, aT, vH, h, dL, ae, eL, iU, aW, Lf, ff, r, en, bu, t, ik, bz, Zj, ck, gk, gn, k, bt, B); #c1 (CCNA1) c2(XP_011533599) c3(cyclin-A1 isoform X1) c4(329702) c5(329703, 329704, 329705, 329706, 329707, 329708, 329709, 329710) c6( ) c7(T, gU, if, al, n, oY, fr, ci, qN, Ul, q, ad, x, pp, aDA, aT, vH, h, dL, ae, eL, iU, aW, Lf, ff, r, en, bu, t, iK, bz, Zj, ck, gk, gn, k, bt, B); #c1 (CCNA2) c2(NP_001228) c3(cyclin-A2) c4(83995) c5(83996, 83997, 83998, 83999, 84000, 84001) c6( ) c7(gh, T, dD, id, ea, aA, fd, wV, eq, ci, nA, C, bX, fr, eW, ad, er, hX, dP, afb, cc, zw, aT, cy, dL, K, jj, vx, aDU, ae, Lg, lR, aW, ev, aDV, eH, LZ, aq, vt, aF, g, cl, eF, t, iR, LB, ck, kT, sS, lt, eM, eD, ll, gZ, at, np, m, k, zu, bt, B, y); #c1(CCNB1) c2(NP_114172) c3(G2/mitotic-specific cyclin-B1) c4(222475, 222476, 222477) c5(222478, 222479, 222480, 222481, 222482, 222483, 222484, 222485, 222486, 222487, 222488, 222489, 222490, 222491, 222492, 222493, 222494, 222495) c6( ) c7(T, fM, jp, aZ, if, aA, bB, n, fd, BF, ci, qN, fr, q, ad, eA, dP, jz, kH, aT, lG, ey, K, bq, hu, fb, aDW, ae, nJ, Lg, jf, kl, d, je, s, qt, aW, ff, eH, r, aq, aF, en, afs, cl, t, eF, bz, KM, iR, ck, bF, KT, iM, bP, eM, dB, bE, hL, m, bt, it, B); #c1(CCNB2) c2(NP_004692) c3(G2/mitotic-specific cyclin-B2) c4(148851) c5(148852, 148853, 148854, 148855, 148856, 148857) c6( ) c7(av, yo, iu, s, eM, qt, aW, ff, aT, hR, aF, ey, ci, en, mo, g, t, bz, eA); #c1(CCNB3) c2(NP_391990) c3(G2/mitotic-specific cyclin-B3 isoform 1) c4(229052) c5(229053, 229054, 229055, 229056, 229057, 229058, 229059, 229060, 229061, 229062, 229063, 229064, 229065, 229066, 229067, 229068, 229069, 229070, 229071, 229072, 229073, 229074) c6( ) c7(aiG, T, eF, dx); #c1(CCNB3) c2(XP_011529134) c3(G2/mitotic-specific cyclin-B3 isoform X1) c4(324458, 324459) c5(324460, 324461, 324462, 324463, 324464, 324465, 324466, 324467, 324468, 324469, 324470, 324471, 324472, 324473, 324474, 324475, 324476, 324477, 324478, 324479, 324480, 324481) c6( ) c7(aiG, T, eF, dx); #c1(CCNB3) c2(XP_011529135) c3(G2/mitotic-specific cyclin-B3 isoform X3) c4( ) c5(324482, 324483, 324484, 324485, 324486, 324487) c6( ) c7(aiG, T, eF, dx); #c1(CCNC) c2(NP_001013417) c3(cyclin-C isoform b) c4( ) c5(25519, 25520, 25521, 25522, 25523, 25524, 25525, 25526, 25527, 25528, 25529, 25530, 25531) c6( ) c7(x, T, af, bE, Y, vA, ev, eH, bB, ey, fr, eF, B); #c1(CCNC) c2(NP_005181) c3(cyclin-C isoform a) c4(153673, 153674, 153675) c5(153676, 153677, 153678, 153679, 153680, 153681, 153682, 153683) c6( ) c7(x, T, af, bE, Y, vA, ev, eH, bB, ey, fr, eF, B); #c1(CCNC) c2(XP_006715657) c3(cyclin-C isoform X1) c4( ) c5(289700) c6( ) c7(x, T, af, bE, Y, vA, ev, eH, bB, ey, fr, eF, B); #c1(CCNC) c2(XP_011534534) c3(cyclin-C isoform X2) c4(331052) c5(331053, 331054, 331055, 331056, 331057, 331058) c6( ) c7(x, T, af, bE, Y, vA, ev, eH, bB, ey, fr, eF, B); #c1(CCND1) c2(NP_444284) c3(Gl/S-specific cyclin-DI) c4(230303) c5(230304, 230305, 230306, 230307, 230308, 230309, 230310, 230311, 230312, 230313, 230314, 230315) c6( ) c7(Fv, gh, G, T, bM, aC, Tb, aEn, pf, if, al, aEf, fd, bB, aeu, ci, auu, ale, ip, x, Vp, afr, aE, OG, axy, dE, aEg, aol, pX, SV, aok, Qk, ja, iT, K, aDZ, hu, pk, dF, eL, Zm, hm, de, eG, yo, zQ, iE, JI, aW, aEI, Ls, az, fc, LZ, Qm, aB, g, as, eF, cm, bw, Qx, fT, aDX, nB, avZ, nF, AM, go, vA, Gs, eM, cj, PV, iS, cx, azg, k, m, gn, Th, Le, YS, iu, cE, aEj, lT, sw, zt, Or, ht, aDT, NT, qy, iB, n, awr, aw, fr, qN, zP, Bl, ft, jz, hw, Yc, pe, Nn, gd, kH, h, lG, jy, hn, dL, aEo, eV, pi, dx, apc, aoX, PX, aAH, AQ, ge, alt, aq, fe, kp, yq, Og, mo, Ca, XT, op, Ld, hq, Ye, iO, WA, ok, YE, bP, GU, dB, hk, anZ, PI, eD, Qu, hL, Vy, gw, hR, at, YH, nz, aEc, it, B, cn, L, fM, AS, eR, jp, yk, eX, aA, hy, v, j, aoN, Qo, aEb, AO, pd, q, eA, qH, zn, eJ, aeZ, XX, RI, lg, hW, nv, LW, qK, aT, gz ae, aqC, jf, s, vT, iU, oV, Y, ev, aEk, xR, r, eS, en, aom, aDY, t, hf, ho, aoy, f, cl, ik, Cv, ov, ck, sS, rR, Dy, nM, iM, aEp, QG, au, RW, wR, aor, zu, bt, rA, VB, hS, iW, GI, hl, kh, aEd, id, pY, Mb, aEa, Dd, aEh, aZ, aD, YM, rQ, vG, oR, DE, dG, ah, Gn, jh, adg, OH, iJ, ad, afb, hX, Jm, dP, aEi, hd, fX, aop, ib, db, oz, av, py, qh, hP, Od, yh, fb, iL, aAL, iA, d, lx, BO, aus, qt, je, ff, eH, Of, cr, aeV, Ej, aoW, aF, anW, gW, aEm, HI, KM, bz, iR, YR, iV, LB, es, gk, eU, RV, pc, amS, bF, bE, auZ, X, cP, bC, ei, y, aed, KS, ne, Ex); #c1(CCND1) c2(XP_006718716) c3(GI/S-specific cyclin-D1 isoform X1) c4(291993) c5( ) c6( ) c7(Fv, gh, G, T, bM, aC, Tb, aEn, pf, if, al, aEf, fd, bB, aeu, ci, auu, ale, ip, x, Vp, afr, aE, OG, axy, dE, aEg, aoL, pX, SV, aok, Qk, ja, iT, K, aDZ, hu, pk, dF, eL, Zm, hm, de, eG, yo, zQ, iE, JI, aW, aEI, Ls, az, fc, LZ, Qm, aB, g, as, eF, cm, bw, Qx, fT, aDX, nB, avZ, nF, AM, go, vA, Gs, eM, cj, PV, iS, cX, azg, k, m, gn, Th, Le, YS, iu, cE, aEj, lT, sw, zt, Or, ht, aDT, NT, qy, iB, n, awr, aw, fr, qN, zP, Bl, ft, jz, hw, Yc, pe, Nn, gd, kH, h, lG, jy, hn, dl, aEo, eV, pi, dx, apc, aoX, PX, aAH, AQ, ge, alt, aq, fe, kp, yq, Og, mo, Ca, XT, op, Ld, hq, Ye, iO, WA, ok, YE, bP, GU, dB, hk, anZ, PI, eD, Qu, hL, Vy, gw, hR, at, YH, nz, aEc, it, B, cn, L, fM, AS, eR, jp, yk, eX, aA, hy, v, j, aoN, Qo, aEb, AO, pd, q, eA, qH, zn, eJ, aeZ, XX, RI, lg, hW, nv, LW, qK, aT, gz, ae, aqC, jf, s, vT, iU, oV, Y, ev, aEk, xR, r, eS, en, aom, aDY, t, hf, ho, aoy, f, cl, ik, Cv, ov, ck, sS, rR, Dy, nM, iM, aEp, QG, au, RW, wR, aor, zu, bt, rA, VB, hS, iW, GI, hl, kh, aEd, id, pY, Mb, aEa, Dd, aEh, aZ, aD, YM, rQ, vG, oR, DE, dG, ah, Gn, jh, adg, OH, iJ, ad, afb, hX, Jm, dP, aEi, hd, fX, aop, ib, db, oz, av, py, qh, hP, Od, yh, fb, iL, aAL, iA, d, lx, BO, aus, qt, je, ff, eH, Of, cr, aeV, Ej, aoW, aF, anW, gW, aEm, HI, KM, bz, iR, YR, iV, LB, es, gk, eU, RV, pc, amS, bF, bE, auZ, X, cP, bC, ei, y, aed, KS, ne, Ex); #c1(CCND2) c2(NP_001750) c3(Gl/S-specific cyclin-D2) c4(120741) c5(120742, 120743, 120744, 120745, 120746, 120747) c6( ) c7(bL, T, id, dA, qy, aA, fd, n, jh, j, ci, uE, q, ad, er, hX, eA, bv, Bl, ft, hd, eJ, aE, av, pe, mj, cF, BJ, kH, aT, x, hn, Od, nJ, ae, jf, PM, B, iU, vT, aW, ff, Of, iw, r, nY, aq, Og, kp, aF, en, hf, dw, t, zE, bu, g, cm, Ld, iP, ck, qJ, eM, nB, dB, eD, cj, aor, m, k, MC, bt, X, hS); #c1(CCND3) c2(NP_001129489) c3(Gl/S-specific cyclin-D3 isoform 1) c4(51608, 51609, 51610) c5(51611, 51612, 51613, 51614, 51615, 51616, 51617, 51618, 51619, 51620, 51621, 51622, 51623, 51624) c6( ) c7(T, fM, jh, fd, n, fr, ci, Ea, zP, q, ad, eA, hd, cq, aE, QJ, aT, dL, bq, pk, fb, ae, af, dx, el, d, s, aW, Y, ff, az, r, Ej, kp, en, cl, g, t, bw, bz, fq, ck, bP, awC, nB, hL, at, bt, B); #c1(CCND3) c2(NP_001129597) c3(G1/S-specific cyclin-D3 isoform 3) c4(51968) c5(51969, 51970, 51971, 51972, 51973, 51974, 51975) c6( ) c7(T, fM, jh, fd, n, fr, ci, Ea, zP, q, ad, eA, hd, cq, aE, QJ, aT, dL, bq, pk, fb, ae, af, dx, eL, d, s, aW, Y, ff, az, r, Ej, kp, en, cl, g, t, bw, bz, fq, ck, bP, awC, nB, hL, at, bt, B); #c1 (CCND3) c2(NP_001274356) c3(Gl/S-specific cyclin-D3 isoform 5) c4(106257, 106258) c5(106259, 106260, 106261, 106262, 106263, 106264, 106265, 106266, 106267, 106268) c6( ) c7(T, fM, jh, fd, n, fr, ci, Ea, zP, q, ad, eA, hd, cq, aE, QJ, aT, dL, bq, pk, fb, ae, af, dx, eL, d, s, aW, Y, ff, az, r, Ej, kp, en, cl, g, t, bw, bz, fq, ck, bP, awC, nB, hL, at, bt, B); #c1(CCND3) c2(NP_001274363) c3(G1/S-specific cyclin-D3 isoform 4) c4( ) c5(106276, 106277, 106278, 106279, 106280, 106281, 106282, 106283, 106284) c6( ) c7(T, fM, jh, fd, n, fr, ci, Ea, zP, q, ad, eA, hd, cq, aE, QJ, aT, dL, bq, pk, fb, ae, af, dx, eL, d, s, aW, Y, ff, az, r, Ej, kp, en, cl, g, t, bw, bz, fq, ck, bP, awC, nB, hL, at, bt, B); #c1(CCND3) c2(NP_001751) c3(GI/S-specific cyclin-D3 isoform 2) c4(120748) c5(120749, 120750, 120751, 120752, 120753, 120754) c6( ) c7(T, fM, jh, fd, n, fr, ci, Ea, zP, q, ad, eA, hd, cq, aE, QJ, aT, dL, bq, pk, fb, a, af, dx, eL, d, s, aW, Y, ff, az, r, Ej, kp, en, cl, g, t, bw, bz, fq, ck, bP, awC, nB, hL, at, bt, B); #c1(CCND3) c2(XP_011513274) c3(G1/S-specific cyclin-D3 isoform X2) c4( ) c5(305546, 305547, 305548, 305549, 305550) c6( ) c7(T, fM, jh, fd, n, fr, ci, Ea, zP, q, ad, eA, hd, cq, aE, QJ, aT, dL, bq, pk, fb, ae, af, dx, eL, d, s, aW, Y, ff, az, r, Ej, kp, en, cl, g, t, bw, bz, fq, ck, bP, awC, nB, hL, at, bt, B); #c1 (CCNDBP1) c2(NP_036274) c3(cyclin-Di-binding protein 1) c4(172802, 172803, 172804) c5(172805, 172806, 172807, 172808, 172809, 172810, 172811, 172812, 172813, 172814, 172815, 172816, 172817, 172818, 172819, 172820, 172821, 172822) c6( ) c7(B, bP, s, jp, bE, aT, bB, aq, ci, bz, X); #c1(CCNE1) c2(NP_001229) c3(Gl/S-specific cyclin-E1) c4(85756, 85757, 85758) c5( ) c6( ) c7(T, aC, jh, cz, fd, n, ci, zP, eA, pX, aT, lG, dL, K, eL, jf, x, d, B, aW, ff, eH, fe, aEq, en, cl, g, t, cM, eF, cm, ck, ac, nB, m, avr, X); #c1 (CCNE1) c2(XP_006723520) c3(G1/S-specific cyclin-E1 isoform X2) c4( ) c5(295223, 295224, 295225, 295226, 295227) c6( ) c7(T, aC, jh, cz, fd, n, ci, zP, eA, pX, aT, lG, dL, K, eL, jf, xQ, d, B, aW, ff, eH, fe, aEq, en, cl, g, t, cM, eF, cm, ck, ac, nB, m, avr, X); #c1(CCNE2) c2(NP_477097) c3(Gl/S-specific cyclin-E2) c4(230743, 230744, 230745) c5(230746, 230747, 230748, 230749, 230750, 230751, 230752, 230753, 230754, 230755, 230756, 230757, 230758) c6( ) c7(ck, dP, T, kl, Dh, aW, bE, aT, bB, lo, m, t, bt, ae, ad); #c1(CCNE2) c2(XP_011515667) c3(Gl/S-specific cyclin-E2 isoform X1) c4( ) c5(308514, 308515, 308516, 308517, 308518, 308519, 308520, 308521, 308522) c6( ) c7(ck, dP, T, kl, Dh, aW, bE, aT, bB, lo, m, t, bt, ae, ad); #c1(CCNF) c2(NP_001752) c3(cyclin-F) c4(120755, 120756) c5(120757, 120758, 120759, 120760, 120761, 120762, 120763, 120764, 120765, 120766, 120767, 120768) c6( ) c7(aW, T, aT); #c1(CCNG1) c2(NP_954854) c3(cyclin-G1) c4(261368, 261369, 261370, 261371, 261372, 261373) c5(261374, 261375, 261376, 261377, 261378, 261379, 261380, 261381, 261382, 261383, 261384, 261385, 261386, 261387, 261388, 261389, 261390, 261391, 261392, 261393, 261394, 261395, 261396, 261397, 261398, 261399, 261400, 261401, 261402, 261403, 261404, 261405, 261406) c6( ) c7(ck, av, T, vA, eH, Az, Xg, lG, n, aq, np, ci, cl, eF, t, B, op, hX); #c1 (CCNG1) c2(XP_011532987) c3(cyclin-Gl isoform X1) c4( ) c5(329216, 329217, 329218, 329219, 329220, 329221, 329222, 329223, 329224, 329225, 329226, 329227, 329228, 329229) c6( ) c7(ck, av, T, vA, eH, Az, Xg, lG, n, aq, np, ci, cl, eF, t, B, op, hX); #c1(CCNG2) c2(XP_011530701) c3(cyclin-G2 isoform X1) c4(326774, 326775, 326776) c5(326777, 326778, 326779, 326780, 326781, 326782, 326783, 326784, 326785, 326786, 326787, 326788, 326789, 326790, 326791, 326792, 326793, 326794, 326795, 326796, 326797, 326798, 326799, 326800, 326801, 326802) c6( ) c7(es, T, cF, d, iA, aW, ht, mg, aT, cm, fd, fr, m, hu, t, eA, y); #c1(CCNH) c2(NP_00186118) c3(cyclin-H isoform 2) c4(77184) c5(77185, 77186, 77187, 77188, 77189) c6( ) c7(bL, T, fM, eR, bB, ci, zP, V, eA, ip, av, Qk, aT, dl, fb, eL, zQ, ff, Pg, nY, vs, en, bz, fq, ek, bJ, ck, bF, eM, dB, bE, cj, at, bC, bD, bt); #c1(CCNH) c2(NP_001230) c3(cyclin-H isoform 1) c4( ) c5( ) c6( ) c7(bL, T, fM, eR, bB, ci, zP, V, eA, ip, av, Qk, aT, dL, fb, eL, zQ, ff, Pg, nY, vs, en, bz, fq, ek, bJ, ck, bF, eM, dB, bE, cj, at, bC, bD, bt); #c1(CCNH) c2(XP_005248686) c3(cyclin-H isoform X3) c4(268512) c5(268513, 268514, 268515, 268516, 268517, 268518) c6( ) c7(bL, T, fM, eR, bB, ci, zP, V, eA, ip, av, Qk, aT, dl, fb, eL, zQ, ff, Pg, nY, vs, en, bz, fq, ek, bJ, ck, bF, eM, dB, bE, cj, at, bC, bD, bt); #c1(CCN1) c2(NP_006826) c3(cyclin-l) c4(168460, 168461, 168462, 168463) c5(168464, 168465, 168466, 168467, 168468, 168469, 168470, 168471, 168472, 168473, 168474, 168475, 168476, 168477, 168478, 168479, 168480, 168481) c6( ) c7(eF, kl, af, ci, eH); #c1(CCN1) c2(XP_011529857) c3(cyclin-1 isoform X1) c4( ) c5(325640, 325641, 325642, 325643, 325644, 325645) c6( ) c7(eF, kl, af, ci, eH); #c1(CCNJ) c2(NP_001127847) c3(cyclin-J isoform 1) c4(49374) c5(49375, 49376, 49377, 49378, 49379, 49380) c6( ) c7(aW, aT); #c1(CCNJ) c2(NP_001127848) c3(cyclin-J isoform 3) c4(49381) c5(49382, 49383, 49384, 49385, 49386, 49387) c6( ) c7(aW, aT); #c1 (CCNJ) c2(NP_061957) c3(cyclin-J isoform 2) c4(201300) c5(201301, 201302, 201303, 201304, 201305, 201306, 201307, 201308, 201309, 201310) c6( ) c7(aW, aT); #c1(CCNJL) c2(NP_078841) c3(cyclin-J-like protein) c4(215668) c5(215669, 215670, 215671, 215672, 215673, 215674) c6( ) c7(cd); #c1(CCNJL) c2(XP_011532950) c3(cyclin-J-like protein isoform X5) c4(329113) c5(329114, 329115, 329116, 329117, 329118, 329119) c6( ) c7(cd); #c1(CCNK) c2(XP_011535577) c3(cyclin-K isoform X1) c4(332719, 332720, 332721, 332722, 332723, 332724) c5(332725, 332726, 332727, 332728, 332729, 332730, 332731, 332732, 332733, 332734, 332735, 332736, 332737, 332738, 332739, 332740, 332741, 332742, 332743, 332744, 332745, 332746, 332747, 332748, 332749, 332750, 332751, 332752, 332753, 332754, 332755, 332756, 332757, 332758, 332759, 332760, 332761, 332762, 332763) c6( ) c7(fd); #c1(CCNL1) c2(NP_064703) c3(cyclin-L1) c4(203480, 203481) c5(203482, 203483, 203484, 203485, 203486, 203487) c6( ) c7(bL, ff, aql, ey, ci, K, en, iK); #c1(CCNL1) c2(XP_005247707) c3(cyclin-LI isoform X5) c4( ) c5(267946) c6( ) c7(bL, ff, aql, ey, ci, K, en, ik); #c1(CCNL1) c2(XP_005247708) c3(cyclin-LI isoform X6) c4( ) c5(267947, 267948, 267949, 267950, 267951, 267952) c6( ) c7(bL, ff, aql, ey, ci, K, en, iK); #c1(CCNL2) c2(XP_011540521) c3(cyclin-L2 isoform X6) c4( ) c5(338989) c6( ) c7(B); #c1(CCNL2) c2(XP_011540523) c3(cyclin-L2 isoform X7) c4(338990) c5(338991, 338992, 338993, 338994, 338995) c6( ) c7(B); #c1(CCNO) c2(NP_066970) c3(cyclin-0) c4(207885) c5(207886, 207887, 207888, 207889, 207890, 207891) c6( ) c7(De, T, d, dB, zb, ap, aT, h, gA, zA, aa); #c1(CCNT1) c2(NP_001231) c3(cyclin-Tl isoform a) c4(87759) c5( ) c6( ) c7(cc, fM, ds, jh, h, as, bq, cm); #c1(CCNY) c2(NP_001269781) c3(cyclin-Y isoform 3) c4(102752) c5(102753, 102754, 102755, 102756, 102757, 102758) c6( ) c7(T, iV, A, eM, ff, ge, fe, en, an, g, bz, af); #c1(CCNY) c2(NP_001269783) c3(cyclin-Y isoform 5) c4( ) c5(102759, 102760, 102761, 102762, 102763, 102764) c6( ) c7(T, iV, A, eM, ff, ge, fe, en, an, g, bz, af); #c1 (CCNY) c2(NP_659449) c3(cyclin-Y isoform 1) c4(239965) c5(239966, 239967, 239968, 239969, 239970, 239971, 239972, 239973, 239974, 239975, 239976, 239977) c6( ) c7(T, iV, A, eM, ff, ge, fe, en, an, g, bz, af); #c1(CCNY) c2(XP_006717455) c3(cyclin-Y isoform X3) c4(291145, 291146) c5( ) c6( ) c7(T, iV, A, eM, ff, ge, fe, en, an, g, bz, af); #c1(CCNY) c2(XP_011517670) c3(cyclin-Y isoform X1) c4( ) c5(311500, 311501, 311502, 311503, 311504, 311505, 311506, 311507) c6( ) c7(T, iV, A, eM, ff, ge, fe, en, an, g, bz, af); #c1(CCP110) c2(NP_055526) c3(centriolar coiled-coil protein of 110 kDa isoform 2) c4(181755) c5(181756, 181757, 181758, 181759, 181760, 18/761, 181762, 181763, 181764, 181765) c6( ) c7(dz, et); #c1(CCP110) c2(XP_011544294) c3(centriolar coiled-coil protein of 110 kDa isoform X1) c4(343446) c5(343447, 343448, 343449, 343450, 343451, 343452) c6( ) c7(dz, et); #c1(CCR1) c2(NP_001286) c3(C-C chemokine receptor type 1) c4(112413) c5(112414, 112415, 112416, 1124/7, 112418, 112419) c6( ) c7(bL, bM, T, Dw, lD, dA, fR, aik, eR, fd, bB, fu, uE, V, mC, ad, dP, zw, F, afm, dV, cU, h, ey, ji, hH, nJ, ae, af, aa, ly, Nr, d, A, yx, fO, hl, mQ, eZ, ff, aq, po, hp, en, t, DF, lx, Nq, W, nb, axs, bE, z, AH, wR, Hb, m, yH, fL, zB, fD, B, y); #c1(CCR10) c2(NP_057686) c3(C-C chemokine receptor type 10) c4(192333) c5(192334, 192335, 192336, 192337, 192338, 192339) c6( ) c7(xS, xT, eU, iM, je, xR, xO, Y, fJ, dN, h, lQ, cB, xP); #c1(CCR2) c2(NP_001116513) c3(C-C chemokine receptor type 2 isoform A) c4(43890) c5(43891, 43892, 43893, 43894, 43895, 43896, 43897, 43898, 43899, 43900) c6( ) c7(bM, T, ea, if, fd, gA, Vd, ur, mF, aY, aDI, wa, AW, xE, aCW, dY, aE, aEu, M, Ds, K, GO, dQ, dF, eU, eN, li, aW, mQ, hl, fc, aik, U, cj, m, hh, aDi, qR, de, Ht, dD, aw, cl, aj, fr, qN, cd, zo, ai, eW, h, By, dL, ey, A, anP, sp, fJ, fe, vo, cM, KT, W, aCz, at, qF, cB, B, bL, Dw, eR, fG, jx, aA, cz, v, j, V, eA, J, ga, hb, nX, aCV, aT, ae, af, eu, aCC, oV, fO, en, t, awi, pb, OC, iQ, ac, c, ig, dh, wR, cS, dy, fD, aEr, aEt, jh, vr, uu, fu, eq, ad, wD, dP, F, aEv, acu, Dq, ds, qh, Os, JK, ul, d, cH, cW, ff, cr, eZ, aF, hp, Bu, bz, Hv, LB, Bv, nb, aEs, aS, cP, ei, lf, y); #c1(CCR2) c2(NP_001116868) c3(C-C chemokine receptor type 2 isoform B) c4(44019, 44020) c5(44021, 44022, 44023, 44024, 44025, 44026, 44027, 44028, 44029, 44030, 44031, 44032) c6( ) c7(bM, T, ea, if, fd, gA, Vd, ur, mF, aY, aDI, wa, AW, xE, aCW, dY, aE, aEu, M, Ds, K, GO, dQ, dF, eL, eN, li, aW, mQ, hl, fc, aik, U, cj, m, hh, aDi, qR, de, Ht, dD, aw, cQ, aj, fr, qN, cd, zo, ai, eW, h, By, dl, ey, A, anP, sp, fJ, fe, vo, cM, KT, W, aCz, at, qF, cB, B, bL, Dw, eR, fG, jx, aA, cz, v, j, V, eA, J, ga, hb, nX, aCV, aT, ae, af, eu, aCC, oV, f, en, t, awi, pb, OC, iQ, ac, c, ig, dh, wR, cS, dy, fD, aEr, aEt, jh, vr, uu, fu, eq, ad, wD, dP, F, aEv, acu, Dq, ds, qh, Os, JK, ul, d, cH, cW, ff, cr, eZ, aF, hp, Bu, bz, Hv, LB, Bv, nb, aEs, aS, cP, ei, lf, y); #c1(CCR3) c2(NP_001158152) c3(C-C chemokine receptor type 3 isoform 3) c4(63203) c5(63204, 63205, 63206, 63207, 63208) c6( ) c7(bM, T, kh, dD, Dw, Ke, akc, aDy, ea, fG, aw, ex, iW, cl, v, j, ci, eq, fr, F, hb, dV, h, eV, Ds, dL, JK, hH, eu, aa, Nr, A, je, sp, dN, po, lo, fe, hp, iV, eE, DF, Nq, TA, cT, y, iM, W, ac, aDu, aCT, z, Jp, dh, JM, at, aS, Tu, fD, lf, bt, cB, aEw); #c1(CCR3) c2(NP_001828) c3(C-C chemokine receptor type 3 isoform 1) c4(121534, 121535) c5(121536, 121537, 121538, 121539, 121540, 121541, 121542, 121543, 121544, 121545, 121546, 121547, 121548, 121549, 121550, 121551, 121552, 121553, 121554) c6( ) c7(bM, T, kh, dD, Dw, Ke, akc, aDy, ea, fG, aw, ex, iW, cl, v, j, ci, eq, fr, F, hb, dV, h, eV, Ds, dL, JK, hH, eu, aa, Nr, A, je, sp, dN, po, lo, fe, hp, iV, eE, DF, Nq, TA, cT, y, iM, W, ac, aDu, aCT, z, Jp, dh, JM, at, aS, Tu, fD, lf, bt, cB, aEw); #c1(CCR3) c2(NP_847898) c3(C-C chemokine receptor type 3 isoform 2) c4(253666) c5(253667, 253668, 253669, 253670, 253671, 253672, 253673, 253674, 253675, 253676, 253677, 253678) c6( ) c7(bM, T, kh, dD, Dw, Ke, akc, aDy, ea, fG, aw, ex, iW, cl, v, j, ci, eq, fr, F, hb, dV, h, eV, Ds, dL, JK, hH, eu, aa, Nr, A, je, sp, dN, po, lo, fe, hp, iV, eE, DF, Nq, TA, cT, y, iM, W, ac, aDu, aCT, z, Jp, dh, JM, at, aS, Tu, fD, lf, bt, cB, aEw); #c1(CCR4) c2(NP_005499) c3(C-C chemokine receptor type 4) c4(156844) c5(156845, 156846, 156847, 156848, 156849, 156850) c6( ) c7(bM, fG, ex, jh, CH, cL, V, eA, aR, o, apJ, kH, aT, h, eV, vB, eu, Nr, B, A, xR, je, Y, cr, eZ, aEz, aq, po, hp, eE, aEx, DF, Nq, cT, eU, iM, Jr, vZ, hc, mx, i, y, aEy); #c1(CCR5) c2(NP_000570) c3(C-C chemokine receptor type 5) c4(12795, 12796) c5(12797, 12798, 12799, 12800, 12801, 12802, 12803, 12804, 12805, 12806, 12807, 12808, 12809, 12810, 12811, 12812, 12813, 12814, 12815, 12816) c6( ) c7(ID, bM, T, aC, fR, ea, if, e, al, fd, bB, cL, gA, qj, ci, aY, aR, aEI, aEQ, AW, dY, aE, Bc, rw, aEJ, dE, dV, Ds, azm, aaW, dQ, hH, eL, bc, cp, aa, Ok, ly, aEV, zQ, aW, aEH, asD, hl, aDn, hN, vs, aoD, g, pl, ve, as, eE, cm, aEP, ba, ou, y, aDs, nB, dc, uy, aiM, hh, qR, iu, dD, aw, fN, n, cQ, qN, qW, cd, ai, eW, ke, cc, zw, gd, aEB, cU, h, dL, ey, eV, ux, aDp, aEL, afu, A, anP, sp, fJ, lb, ge, fe, aq, CA, kk, cM, lx, KT, aCz, dB, NL, hk, aES, bm, at, uz, aEG, eo, aDE, agy, gg, Pr, B, abW, aEC, vQ, bL, Dw, cB, dA, eR, oe, jx, ex, cz, hy, v, j, aDO, V, q, o, eA, eJ, ady, ga, aEE, jZ, aT, cy, aDt, sX, ae, af, eu, aEF, oV, aEM, aCH, fO, Y, bg, r, iy, lo, aER, iK, vk, ck, iQ, aEA, ac, ig, AH, fP, auT, wR, rK, yH, dy, bt, Kd, Gl, kh, Ke, qb, uG, gN, aEK, vr, jh, aET, ah, jE, aDz, fu, EA, eq, ad, wD, LH, dP, hX, F, fX, od, aEv, oz, acu, ds, qh, HD, JK, vB, ji, uD, nJ, ul, d, yx, cr, aEO, rM, Mf, hv, eZ, hp, aEW, Fq, iV, Hv, zr, axs, bE, atM, z, do, uR, aED, aDg, aS, aEU, aEN, ei, lf, ne, rk); #c1(CCR6) c2(NP_004358) c3(C-C chemokine receptor type 6) c4(145640) c5(145641, 145642, 145643, 145644, 145645, 145646, 145647, 145648, 145649, 145650, 145651, 145652, 145653, 145654) c6( ) c7(bM, T, aC, aAz, aD, jh, fd, v, qj, fu, Qp, q, ad, ai, eA, ip, av, iu, hd, aE, pN, aT, h, ey, ji, hH, ae, DA, je, xR, aW, md, hl, HA, r, cr, fe, vs, aF, yq, iV, eE, cm, bz, pb, fT, lt, aDv, zr, nB, AM, hk, hc, DW, eM, iM, at, wR, fD, y, Pr, B); #c1(CCR7) c2(NP_001829) c3(C-C chemokine receptor type 7 precursor) c4(121555) c5(121556, 121557, 121558, 121559, 121560, 121561) c6( ) c7(ID, T, os, aC, fR, ea, fd, bB, cL, ci, Qp, x, aE, aEX, dV, K, hH, bc, ly, Jl, aW, aoo, g, bw, iP, lt, eM, nB, iT, tV, cj, dW, k, m, iu, dD, JC, gp, fN, n, cd, br, eW, BI, kH, h, eV, KV, A, MD, lb, pm, lx, Wt, ajd, cB, B, it, dA, eX, aA, v, j, V, pd, q, o, eA, aT, AJ, dz, jj, ae, iU, iZ, Y, Pg, r, en, cl, t, ik, pb, ck, nM, iM, oX, bt, iW, DA, bs, jh, oZ, eq, hF, ad, iC, av, hd, F, oW, pq, je, lx, ff, cr, eZ, aF, iV, bz, LB, eU, bE, eB, lC, KS, X); #c1(CCR8) c2(NP_005192) c3(C-C chemokine receptor type 8) c4(153798, 153799) c5(153800, 153801, 153802, 153803, 153804, 153805, 153806, 153807, 153808, 153809, 153810, 153811) c6( ) c7(dP, bM, JZ, fG, cj, h, vB, fD, eE, cB, DF); #c1(CCR9) c2(NP_001243298) c3(C-C chemokine receptor type 9 isoform B) c4(90386) c5(90387, 90388, 90389, 90390, 90391, 90392, 90393, 90394) c6( ) c7(bM, xN, T, dD, ea, iW, j, eq, ad, dP, x, aT, kH, qU, h, ae, xQ, je, iZ, aW, Y, mQ, eZ, hp, t, ck, iM, z, m, k, fD, y, cB); #c1(CCR9) c2(XP_011531614) c3(C-C chemokine receptor type 9 isoform X1) c4(327749) c5(327750, 327751, 327752, 327753, 327754, 327755, 327756, 327757) c6( ) c7(bM, xN, T, dD, ea, iW, j, eq, ad, dP, x, aT, kH, qU, h, ae, xQ, je, iZ, aW, Y, mQ, eZ, hp, t, ck, iM, z, m, k, fD, y, cB); #c1(CCRL2) c2(NP_001124382) c3(C-C chemokine receptor-like 2 isoform 2) c4(48741, 48742, 48743) c5(48744, 48745, 48746, 48747, 48748, 48749, 48750, 48751, 48752, 48753) c6( ) c7(bM, T, aD, e, NT, fd, v, fu, q, ad, ai, wD, av, iu, hd, cF, aE, h, ey, lQ, ji, hu, hH, ae, iM, iQ, DA, aCr, je, aCq, xR, HA, hl, cr, r, fe, yq, hp, iV, eE, bz, pb, fT, yl, lt, zr, Jr, nB, hk, hc, do, AM, aDv, at, wR, Pk, Pr, B, y); #c1(CCRL2) c2(NP_003956) c3(C-C chemokine receptor-like 2 isoform 1) c4( ) c5(142428, 142429, 142430, 142431, 142432, 142433, 142434, 142435, 142436, 142437, 142438, 142439, 142440, 142441, 142442, 142443) c6( ) c7(bM, T, aD, e, NT, fd, v, fu, q, ad, ai, wD, av, iu, hd, cF, aE, h, ey, lQ, ji, hu, hH, ae, iM, iQ, DA, aCr, je, aCq, xR, HA, hl, cr, r, fe, yq, hp, iV, eE, bz, pb, fT, yl, lt, zr, Jr, nB, hk, hc, do, AM, aDv, at, wR, Pk, Pr, B, y); #c1 (CCRN4L) c2(NP_036250) c3(nocturnin) c4(172634) c5(172635, 172636, 172637, 172638, 172639, 172640) c6( ) c7(bM, T, ex, CH, cL, v, V, q, aR, o, av, kH, h, eV, vB, Nr, A, je, xR, Y, fc, cr, r, aEz, eZ, po, eE, aEx, DF, Nq, cT, eU, iM, Jr, vZ, hc, mx, pP, cP, i, y, aEy); #c1 (CCS) c2(NP_005116) c3(copper chaperone for superoxide dismutase) c4(153024) c5(153025, 153026, 153027, 153028, 153029, 153030, 153031) c6( ) c7(eY, ac, c, Y, bW, aEY, r, acJ, PJ, q, aya, Qt); #c1(CCSER1) c2(NP_001138537) c3 (serine-rich coiled-coil domain-containing protein 1 isoform 1) c4(56971, 56972) c5(56973, 56974, 56975, 56976, 56977, 56978, 56979, 56980, 56981, 56982) c6( ) c7(T, aE, oe, cu, Ea, sj); #c1(CCSER1) c2(NP_997374) c3(serine-rich coiled-coil domain-containing protein 1 isoform 2) c4(265473) c5(265474, 265475, 265476, 265477, 265478, 265479, 265480, 265481, 265482, 265483, 265484, 265485) c6( ) c7(T, aE, oe, cu, Ea, sj); #c1(CCT2) c2(NP_006422) c3(T-complex protein 1 subunit beta isoform 1) c4(164906, 164907) c5(164908, 164909, 164910, 164911, 164912, 164913, 164914, 164915, 164916, 164917, 164918, 164919) c6( ) c7(ac, bA); #c1(CCT3) c2(NP_001008800) c3(T-complex protein 1 subunit gamma isoform c) c4(23423, 23424) c5(23425, 23426, 23427, 23428, 23429, 23430, 23431, 23432, 23433, 23434, 23435, 23436) c6( ) c7(B, U, r, h, q, dr, dR); #c1(CCT3) c2(NP_005989) c3(T-complex protein 1 subunit gamma isoform a) c4(161018, 161019, 161020, 161021) c5(161022, 161023, 161024, 161025, 161026, 161027, 161028, 161029, 161030, 161031, 161032, 161033, 161034, 161035, 161036, 161037, 161038, 161039, 161040, 161041, 161042, 161043, 16104, 161045, 161046, 161047, 161048, 161049) c6( ) c7(B, U, r, h, q, dr, dR); #c1(CCT4) c2(NP_001243650) c3(T-complex protein 1 subunit delta isoform b) c4(91308) c5(91309, 91310, 91311, 91312, 91313, 91314, 91315, 91316, 91317, 91318, 91319, 91320) c6( ) c7(cm, Pt); #c1(CCT4) c2(NP_006421) c3(T-complex protein 1 subunit delta isoform a) c4(164891, 164892, 164893) c5(164894, 164895, 164896, 164897, 164898, 164899, 164900, 164901, 164902, 164903, 164904, 164905) c6( ) c7(cm, Pt); #c1(CCT5) c2(NP_036205) c3(T-complex protein 1 subunit epsilon) c4(172358, 172359) c5(172360, 172361, 172362, 172363, 172364, 172365, 172366, 172367, 172368, 172369, 172370, 172371, 172372, 172373, 172374, 172375, 172376, 172377) c6( ) c7(cZ, lu, xZ, aFa, Pt, Be, aT, r, h, ey, aEZ, q, Qn); #c1(CCT6A) c2(NP_001009186) c3(T-complex protein 1 subunit zeta isoform b) c4(23573) c5(23574, 23575, 23576, 23577, 23578, 23579, 23580, 23581, 23582, 23583, 23584) c6( ) c7(ff, q, en, r); #c1(CCT6A) c2(NP_001753) c3(T-complex protein 1 subunit zeta isoform a) c4(120769, 120770, 120771) c5(120772, 120773, 120774, 120775, 120776, 120777, 120778, 120779, 120780, 120781, 120782, 120783, 120784) c6( ) c7(ff, q, en, r); #c1(CCT6B) c2(NP_001180458) c3(T-complex protein 1 subunit zeta-2 isoform 2) c4(74117) c5(74118, 74119, 74120, 74121, 74122, 74123) c6( ) c7(pk, T); #c1(CCT6B) c2(NP_001180459) c3(T-complex protein 1 subunit zeta-2 isoform 3) c4(74/24) c5(74125, 74126, 74127, 74128, 74129, 74130) c6( ) c7(pk, T); #c1(CCT6B) c2(NP_006575) c3(T-complex protein 1 subunit zeta-2 isoform 1) c4(166336) c5(166337, 166338, 166339, 166340, 166341, 166342) c6( ) c7(pk, T); #c1(CCT7) c2(NP_001009570) c3(T-complex protein 1 subunit eta isoform b) c4( ) c5(23625, 23626, 23627, 23628, 23629, 23630) c6( ) c7(aw, aA, h, r, mq, mJ, q); #c1(CCT7) c2(NP_001159756) c3(T-complex protein 1 subunit eta isoform c) c4(64747) c5(64748, 64749, 64750, 64751, 64752, 64753, 64754, 64755, 64756, 64757, 64758, 64759, 64760, 64761, 64762, 64763, 64764) c6( ) c7(aw, aA, h, r, mq, mJ, q); #c1(CCT7) c2(NP_001159757) c3(T-complex protein 1 subunit eta isoform d) c4(64765) c5( ) c6( ) c7(aw, aA, h, r, mq, mJ, q); #c1(CCT7) c2(NP_006420) c3(T-complex protein 1 subunit eta isoform a) c4(164874, 164875, 164876, 164877) c5(164878, 164879, 164880, 164881, 164882, 164883, 164884, 164885, 164886, 164887, 164888, 164889, 164890) c6( ) c7(aw, aA, h, r, mq, mJ, q); #c1(CD109) c2(NP_001153059) c3(CD109 antigen isoform 2 preproprotein) c4(60388) c5(60389, 60390, 60391, 60392, 60393, 60394) c6( ) c7(T, ft, oe, eH, AQ, RW, aT, h, ff, ci, en, g, eF, y, Wq, X); #c1(CD109) c2(NP_001153060) c3(CD109 antigen isoform 3 preproprotein) c4(60395) c5(60396, 60397, 60398, 60399, 60400, 60401) c6( ) c7(T, ft, oe, eH, AQ, RW, aT, h, ff, ci, en, g, eF, y, Wq, X); #c1(CD109) c2(NP_598000) c3(CD109 antigen isoform 1 preproprotein) c4(235250) c5(235251, 235252, 235253, 235254, 235255, 235256) c6( ) c7(T, ft, oe, eH, AQ, RW, aT, h, ff, ci, en, g, eF, y, Wq, X); #c1(CD14) c2(NP_001167576) c3(monocyte differentiation antigen CD14 precursor) c4(69319) c5(69320, 69321, 69322, 69323, 69324, 69325, 69326, 69327, 69328, 69329) c6( ) c7(oO, bM, T, JN, fR, Js, aFg, ea, hV, e, qx, al, fd, cl, Vd, ci, Jx, Ap, fw, aDI, Tz, ip, NF, AW, xE, aE, rw, Gb, dV, Ds, K, dQ, aFk, dF, eL, eN, hm, aa, yo, aAg, aW, az, hl, asD, md, g, hJ, tU, uC, eE, bw, ba, fT, eM, xD, dc, JA, AV, ki, cj, uo, afp, Dt, eg, Le, aFf, qR, Wx, FW, dD, sQ, zt, aw, ht, aFc, n, fr, jG, pM, aCQ, TI, cd, zo, ai, TB, eW, ec, jL, aFq, cU, h, eV, dL, aaA, aFi, wb, ey, aDp, wC, A, mR, oo, sp, fe, nN, jT, cM, aFj, Wt, asE, ar, kT, aBs, JM, at, uz, Dp, aEG, ut, fl, aDE, eo, B, Jo, bL, Dw, fM, dA, eR, jx, eX, aA, tc, ol, v, j, OY, C, aFn, V, afv, q, o, er, cF, apJ, sl, ld, rS, aT, yu, Oi, cy, jj, an, aFm, Cy, ae, af, eu, aFs, s, iU, bg, Pg, r, aFo, lo, ahl, pb, ck, cT, ac, c, ig, wD, hc, gX, tM, mf, cS, dy, JH, fD, Da, zu, bt, ao, Kd, sY, aFe, akd, aD, iW, jh, vr, Jz, jE, ah, eq, iG, ad, kL, hX, Jm, aFu, F, ib, aFl, aFh, Dq, Os, hD, LA, JK, vB, lQ, jo, XI, NM, ul, d, cW, qt, yx, wc, aft, eZ, aFr, gE, hp, aF, Fq, iV, aFd, ek, D, Cq, gk, pc, zr, yE, z, aka, aFb, eB, wz, sG, aS, ei, lf, fh, y); #c1(CD151) c2(NP_001034579) c3(CD151 antigen) c4(31810, 31811, 31812, 31813, 31814, 31815, 31816, 31817) c5(31818, 31819, 31820, 31821, 31822, 31823, 31824, 31825, 31826, 31827, 31828, 31829, 31830, 31831, 31832, 31833, 31834, 31835, 31836, 31837, 31838, 31839, 31840, 31841, 31842, 31843, 31844, 31845, 31846, 31847, 31848, 31849, 31850, 31851, 31852, 31853, 31854, 31855, 31856, 31857, 31858, 31859, 31860, 31861, 31862, 31863, 31864, 31865, 31866, 31867, 31868, 31869, 31870, 31871, 31872, 31873, 31874, 31875, 31876, 31877, 31878, 31879, 31880, 31881) c6( ) c7(T, eR, fG, aw, aA, bB, n, axk, ci, aFw, eA, ad, kH, aT, cy, ae, d, je, LC, Y, aW, ff, aF, en, bz, LB, ck, iM, eM, bE, uo, dy, B); #c1 (CD163) c2(NP_004235) c3(scavenger receptor cysteine-rich type 1 protein M130 isoform a precursor) c4(144501, 144502) c5(144503, 144504, 144505, 144506, 144507, 144508) c6( ) c7(oO, bL, T, EM, dD, eR, ea, aw, qy, aA, n, aj, v, eq, azF, C, pz, q, J, eA, ip, tg, AW, ib, dY, hW, ld, dV, aT, h, dL, ey, K, lM, eL, hZ, nP, iU, iZ, aW, Y, eH, cr, r, fe, aFx, aF, iV, eF, iP, LB, c, zr, Vy, hc, sG, at, m, hh, y, bt); #c1(CD163) c2(NP_981961) c3(scavenger receptor cysteine-rich type 1 protein M130 isoform b precursor) c4(263205) c5(263206, 263207, 263208, 263209, 263210, 263211, 263212, 263213, 263214, 263215, 263216, 263217, 263218) c6( ) c7(oO, bL, T, EM, dD, eR, ea, aw, qy, aA, n, aj, v, eq, azF, C, pz, q, J, eA, ip, tg, AW, ib, dY, hW, ld, dV, aT, h, dL, ey, K, lM, eL, hZ, nP, iU, iZ, aW, Y, eH, cr, r, fe, aFx, aF, iV, eF, iP, LB, c, zr, Vy, hc, sG, at, m, hh, y, bt); #c1(CD163) c2(XP_005253585) c3 (scavenger receptor cysteine-rich type 1 protein M130 isoform X1) c4(272574) c5(272575, 272576, 272577, 272578, 272579, 272580) c6( ) c7(oO, bL, T, EM, dD, eR, ea, aw, qy, aA, n, aj, v, eq, azF, C, pz, q, J, eA, ip, tg, AW, ib, dY, hW, ld, dV, aT, h, dL, ey, K, lM, el, hZ, nP, iU, iZ, aW, Y, eH, cr, r, fe, aFx, aF, iV, eF, iP, LB, c, zr, Vy, hc, sG, at, m, hh, y, bt); #c1(CD163L1) c2(NP_777601) c3(scavenger receptor cysteine-rich type 1 protein M160 isoform 2 precursor) c4(251307) c5(251308, 251309, 251310, 251311, 251312, 251313, 251314, 251315) c6(251316, 251317) c7(Y); #c1(CD163L1) c2(XP_011518918) c3(scavenger receptor cysteine-rich type 1 protein M160 isoform X1) c4(313344) c5(313345, 313346, 313347, 313348, 313349, 313350, 313351, 313352) c6(313353, 313354) c7(Y); #c1(CD163L1) c2(XP_011518919) c3(scavenger receptor cysteine-rich type 1 protein M160 isoform X2) c4(313355) c5( ) c6( ) c7(Y); #c1(CD163L1) c2(XP_011518923) c3(scavenger receptor cysteine-rich type 1 protein M160 isoform X5) c4( ) c5(313356, 313357, 313358, 313359, 313360, 313361, 313362, 313363) c6(313364, 313365) c7(Y); #c1(CD164) c2(NP_001135873) c3(sialomucin core protein 24 isoform 2 precursor) c4(53448) c5(53449, 53450, 53451, 53452, 53453, 53454) c6( ) c7(eU, pg, m, g, t, ad); #c1 (CD164) c2(NP_001135874) c3(sialomucin core protein 24 isoform 3 precursor) c4(53455, 53456) c5(53457, 53458, 53459, 53460, 53461, 53462, 53463, 53464, 53465, 53466, 53467, 53468) c6( ) c7(eU, pg, m, g, t, ad); #c1(CD164) c2(NP_001135875) c3(sialomucin core protein 24 isoform 4 precursor) c4(53469) c5(53470, 53471, 53472, 53473, 53474, 53475) c6( ) c7(eU, pg, m, g, t, ad); #c1(CD164) c2(NP_001135876) c3(sialomucin core protein 24 isoform 5 precursor) c4(53476) c5(53477, 53478, 53479, 53480, 53481, 53482) c6( ) c7(eU, pg, m, g, t, ad); #c1(CD164) c2(NP_006007) c3(sialomucin core protein 24 isoform 1 precursor) c4(16/123) c5(161124, 161125, 161126, 161127, 161128, 16/129) c6( ) c7(eU, pg, m, g, t, ad); #c1(CD(77) c2(NP_065139) c3(CD177 antigen precursor) c4(204223, 204224, 204225) c5(204226, 204227, 204228, 204229, 204230, 204231, 204232, 204233, 204234, 204235, 204236, 204237, 204238, 204239, 204240, 204241, 204242, 204243, 204244, 204245, 204246, 204247) c6( ) c7(bL, gV, T, lD, Ol, jx, jh, aA, bB, fd, ci, C, iJ, eA, ad, eW, oT, Cj, dE, ds, aFy, aT, oW, h, dz, ae, cY, ul, ly, VC, A, d, lx, iU, et, aW, eZ, r, kD, aFC, aq, aF, yq, g, pm, cm, aFB, aFz, iK, mK, lx, dR, aFA, bw, L, BU, pc, age, eM, bE, AH, cj, hR, pg, aS, pw, bt, it, B, aeA); #c1(CD180) c2(NP_005573) c3 (CD180 antigen precursor) c4(157578) c5(157579, 157580, 157581, 157582, 157583, 157584) c6( ) c7(jx, eZ, cj, dq, v, bc, ad); #c1(CD19) c2(NP_001171569) c3(B-lymphocyte antigen CD19 isoform 1 precursor) c4(70632) c5(70633, 70634, 70635, 70636, 70637, 70638) c6( ) c7(bM, T, aFG, fM, dA, iW, jx, eX, jh, fd, aeu, lR, aFF, hF, V, mC, eW, tD, zn, x, aFD, ke, pe, lg, OG, ds, h, eV, hn, ey, ox, jj, ajA, bc, azh, nP, iU, oV, Y, axu, aFE, bg, eZ, Of, bw, Og, lo, hp, aom, aot, kk, cM, cm, pb, iV, Wt, asE, Cv, ck, ou, ba, eM, iT, of, cj, dW, k, aDb, fl, y, bt, Ze, abW); #c1(CD19) c2(NP_001761) c3(B-lymphocyte antigen CD19 isoform 2 precursor) c4(120875, (20876, 120877) c5(120878, 120879, 120880, 120881, 120882, 120883, 120884, 120885, 120886, 120887, 120888, 120889, 120890, 120891, 120892, 120893, 120894, 120895) c6( ) c7(bM, T, aFG, fM, dA, iW, jx, eX, jh, fd, aeu, lR, aFF, hF, V, mC, eW, tD, zn, x, aFD, ke, pe, lg, OG, ds, h, eV, hn, ey, ox, jj, ajA, bc, azh, nP, iU, oV, Y, axu, aFE, bg, eZ, Of, bw, Og, lo, hp, aom, aot, kk, cM, cm, pb, iV, Wt, asE, Cv, ck, ou, ba, eM, iT, of, cj, dW, k, aDb, fl, y, bt, Ze, abW); #c1(CD1A) c2(NP_001754) c3(T-cell surface glycoprotein CD1a precursor) c4(120785) c5(120786, 120787, 120788, 120789, 120790, 120791) c6( ) c7(T, qb, bS, OY, ci, ct, fu, Qp, pd, V, ds, aT, h, rO, dF, A, iU, fJ, Z, BE, aFH, ck, xT, nM, yW, Qw, at, i, bt); #c1(CD1B) c2(NP_001755) c3(T-cell surface glycoprotein CD1b precursor) c4(120792) c5(120793, 120794, 120795, 120796, 120797, 120798) c6( ) c7(T, qb, bO, bB, bS, OY, ct, ci, fu, Qp, V, wD, AW, ds, aT, h, aFl, dF, A, iU, ev, fJ, cr, aw, Z, aFH, eE, ck, xT, BU, nM, yW, bE, Qw, at, eo, i, bt, B); #c1(CD1B) c2(XP_011508421) c3(T-cell surface glycoprotein CD1b isoform X1) c4(299137) c5(299138, 299139, 299140, 299141, 299142, 299143) c6( ) c7(T, qb, bO, bB, bS, OY, ct, ci, fu, Qp, V, wD, AW, ds, aT, h, aFl, dF, A, iU, ev, fJ, cr, aw, Z, aFH, eE, ck, xT, BU, nM, yW, bE, Qw, at, eo, i, bt, B); #c1(CD1C) c2(NP_001756) c3(T-cell surface glycoprotein CD1c precursor) c4(120799, 120800) c5(120801, 120802, 120803, 120804, 120805, 120806, 120807, 120808, 120809, 120810, 120811, 120812) c6( ) c7(T, qb, bS, OY, ci, ct, fu, Qp, hF, uE, V, ds, aT, h, dF, bc, A, iU, oV, fJ, Z, aFH, eE, ck, xT, BU, nM, yW, Qw, cj, at, i, bt); #c1(CD1C) c2(XP_005245636) c3(T-cell surface glycoprotein CD1c isoform X1) c4(266822, 266823) c5(266824, 266825, 266826, 266827, 266828, 266829, 266830, 266831, 266832, 266833, 266834, 266835) c6( ) c7(T, qb, bS, OY, ci, ct, fu, Qp, hF, uE, V, ds, aT, h, dF, bc, A, iU, oV, fJ, Z, aFH, eE, ck, xT, BU, nM, yW, Qw, cj, at, i, bt); #c1(CD1D) c2(NP_001757) c3(antigen-presenting glycoprotein CD1d precursor) c4(120813) c5(120814, 120815, 120816, 120817, 120818, 120819) c6( ) c7(oO, bM, T, JZ, FR, yG, al, jx, fd, jh, n, aj, bS, v, ct, ci, bX, aFJ, mF, V, OY, Qp, aR, J, hX, x, ds, akJ, aT, h, ey, lQ, ji, OM, NM, dF, jf, akV, A, iU, mD, aW, fJ, aFK, cr, fe, aq, lo, yq, hp, aFH, Z, eE, cm, Wt, ck, vU, y, nM, yW, xD, Qw, cj, at, uz, oX, k, i, hE, bt, fh, B, ao); #c1(CD1E) c2(NP_001036048) c3(T-cell surface glycoprotein CD1e, membrane-associated isoform b precursor) c4(33850) c5(33851, 33852, 33853, 33854, 33855, 33856, 33857, 33858, 33859, 33860, 33861, 33862, 33863) c6( ) c7(T, bS, OY, ci, ct, Qp, V, ds, aT, h, A, iU, fJ, eE, Z, aFH, ck, nM, c, yW, Qw, at, i, bt, hv); #c1(CD1E) c2(NP_001036050) c3(T-cell surface glycoprotein CD1e, membrane-associated isoform d precursor) c4(33864) c5(33865, 33866, 33867, 33868, 33869, 33870) c6( ) c7(T, bS, OY, ci, ct, Qp, V, ds, aT, h, A, iU, fJ, eE, Z, aFH, ck, nM, c, yW, Qw, at, i, bt, hv); #c1(CD1E) c2(NP_001036051) c3(T-cell surface glycoprotein CD1e, membrane-associated isoform e precursor) c4(33871) c5(33872, 33873, 33874, 33875, 33876, 33877) c6( ) c7(T, bS, OY, ci, ct, Qp, V, ds, aT, h, A, iU, fJ, eE, Z, aFH, ck, nM, c, yW, Qw, at, i, bt, hv); #c1(CD1E) c2(NP_001036052) c3(T-cell surface glycoprotein CD1e, membrane-associated isoform f precursor) c4(33878) c5(33879, 33880, 33881, 33882, 33883, 33884, 33885, 33886, 33887, 33888, 33889) c6( ) c7(T, bS, OY, ci, ct, Qp, V, ds, aT, h, A, iU, fJ, eE, Z, aFH, ck, nM, c, yW, Qw, at, i, bt, hv); #c1(CD1E) c2(NP_001172036) c3(T-cell surface glycoprotein CD1e, membrane-associated isoform g precursor) c4(72305) c5(72306, 72307, 72308, 72309, 72310, 72311) c6( ) c7(T, bS, OY, ci, ct, Qp, V, ds, aT, h, A, iU, fJ, eE, Z, aFH, ck, nM, c, yW, Ww, at, i, bt, hv); #c1(CD1E) c2(NP_001172037) c3(T-cell surface glycoprotein CD1e, membrane-associated isoform i precursor) c4(72312) c5(72313, 72314, 72315, 72316, 72317, 72318) c6( ) c7(T, bS, OY, ci, ct, Qp, V, ds, aT, h, A, iU, fJ, eE, Z, aFH, ck, nM, c, yW, Qw, at, i, bt, hv); #c1(CD1E) c2(NP_001172039) c3(T-cell surface glycoprotein CD1e, membrane-associated isoform 1 precursor) c4(72319) c5(72320, 72321, 72322, 72323, 72324, 72325) c6( ) c7(T, bS, OY, ci, ct, Op, V, ds, aT, h, A, iU, fJ, eE, Z, aFH, ck, nM, c, yW, Qw, at, i, bt, hv); #c1(CD1E) c2(NP_001172041) c3(T-cell surface glycoprotein CD1e, membrane-associated isoform j precursor) c4(72326) c5(72327, 72328, 72329, 72330, 72331, 72332, 72333) c6( ) c7(T, bS, OY, ci, ct, Qp, V, ds, aT, h, A, iU, fJ, eE, Z, aFH, ck, nM, c, yW, Qw, at, i, bt, hv); #c1(CD1E) c2(NP_001172042) c3(T-cell surface glycoprotein CD1e, membrane-associated isoform k precursor) c4(72334) c5(72335, 72336, 72337, 72338, 72339, 72340) c6( ) c7(T, bS, OY, ci, ct, Qp, V, ds, aT, h, A, iU, fJ, eE, Z, aFH, ck, nM, c, yW, Ww, at, i, bt, hv); #c1(CD1E) c2(NP_001172043) c3(T-cell surface glycoprotein CD1e, membrane-associated isoform m precursor) c4(72341) c5(72342, 72343, 72344, 72345, 72346, 72347) c6( ) c7(T, bS, OY, ci, ct, Qp, V, ds, aT, h, A, iU, fJ, eE, Z, aFH, ck, nM, c, yW, Qw, at, i, bt, hv); #c1(CD1E) c2(NP_001172044) c3(T-cell surface glycoprotein CD1e, membrane-associated isoform h precursor) c4(72348) c5(72349, 72350, 72351, 72352, 72353, 72354) c6( ) c7(T, bS, OY, ci, ct, Qp, V, ds, aT, h, A, iU, fJ, eE, Z, aFH, ck, nM, c, yW, Qw, at, i, bt, hv); #c1(CD1E) c2(NP_112155) c3(T-cell surface glycoprotein CD1e, membrane-associated isoform a precursor) c4(220508, 220509) c5(220510, 220511, 220512, 220513, 220514, 220515) c6( ) c7(T, bS, OY, ci, ct, Op, V, ds, aT, h, A, iU, fJ, eE, Z, aFH, ck, nM, c, yW, Qw, at, i, bt, hv); #c1(CD1E) c2(XP_011508436) c3(T-cell surface glycoprotein CD1e, membrane-associated isoform X1) c4(299150, 299151) c5(299152, 299153, 299154, 299155, 299156, 299157) c6( ) c7(T, bS, OY, ci, ct, Qp, V, ds, aT, h, A, iU, fJ, eE, Z, aFH, ck, nM, c, yW, Qw, at, i, bt, hv); #c1(CD200) c2(NP_001004196) c3(0X-2 membrane glycoprotein isoform b) c4(20484) c5(20485, 20486, 20487, 20488, 20489, 20490) c6( ) c7(ck, bM, T, zw, dA, aW, Y, aw, wL, bt, cj, aT, fd, lo, eo, an, hF, V, X, cp); #c1(CD200) c2(NP_005935) c3(0X-2 membrane glycoprotein isoform a precursor) c4(160533) c5(160534, 160535, 160536, 160537, 160538, 160539) c6( ) c7(ck, bM, T, zw, dA, aW, Y, aw, wL, bt, cj, aT, fd, lo, eo, an, hF, V, X, cp); #c1(CD200) c2(XP_005247547) c3(OX-2 membrane glycoprotein isoform X9) c4( ) c5(267865, 267866) c6( ) c7(ck, bM, T, zw, dA, aW, Y, aw, wL, bt, cj, aT, fd, lo, eo, an, hF, V, X, cp); #c1(CD200R1) c2(NP_620161) c3(cell surface glycoprotein CD200 receptor 1 isoform a precursor) c4(237423) c5(237424, 237425, 237426, 237427, 237428, 237429) c6( ) c7(Ye, ae, T, cF, zw, oz, aW, U, Nt, aT, alX, bt, cl, uu, an, abC, V, alY, ad); #c1(CD200RI) c2(NP_620385) c3(cell surface glycoprotein CD200 receptor 1 isoform b precursor) c4(237536) c5(237537, 237538, 237539, 237540, 237541, 237542) c6( ) c7(Ye, ae, T, cF, zw, oz, aW, U, Nt, aT, alX, bt, cl, uu, an, abC, V, alY, ad); #c1(CD200R1) c2(NP_620386) c3(cell surface glycoprotein CD200 receptor 1 isoform c precursor) c4(237543) c5(237544, 237545, 237546, 237547, 237548, 237549) c6( ) c7(Ye, ae, T, cF, zw, oz, aW, U, Nt, aT, alX, bt, cl, uu, an, abC, V, alY, ad); #c1(CD200RI) c2(NP_740750) c3(cell surface glycoprotein CD200 receptor 1 isoform d precursor) c4(247448) c5(247449, 247450, 247451, 247452, 247453, 247454) c6( ) c7(Ye, ae, T, cF, zw, oz, aW, U, Nt, aT, alX, bt, cl, uu, an, abC, V, alY, ad); #c1(CD207) c2(NP_056532) c3(C-type lectin domain family 4 member K) c4(187971) c5(187972, 187973, 187974, 187975, 187976, 187977) c6( ) c7(Qp, et, dz, fe, aFL); #c1(CD209) c2(NP_001138365) c3(CD209 antigen isoform 5) c4(56383) c5(56384, 56385, 56386, 56387, 56388, 56389) c6( ) c7(bM, Dw, fR, jx, al, fr, aBl, q, aFM, AW, F, kH, h, Ds, wc, hl, ge, r, lo, aDm, hp, yq, aCK, eE, iV, iQ, Jr, ig, AH, y); #c1(CD209) c2(NP_001138366) c3(CD209 antigen isoform 6) c4(56390) c5(56391, 56392, 56393, 56394, 56395, 56396) c6( ) c7(bM, Dw, fR, jx, al, fr, aBl, q, aFM, AW, F, kH, h, Ds, wc, hl, ge, r, lo, aDm, hp, yq, aCK, eE, iV, iQ, Jr, ig, AH, y); #c1(CD209) c2(NP_00138367) c3(CD209 antigen isoform 7) c4(56397) c5(56398, 56399, 56400, 56401, 56402, 56403, 56404, 56405, 56406, 56407) c6( ) c7(bM, Dw, fR, jx, al, fr, aBl, q, aFM, AW, F, KH, h, Ds, wc, hl, ge, r, lo, aDm, hp, yq, aCK, eE, iV, iQ, Jr, ig, AH, y); #c1(CD209) c2(NP_001138368) c3(CD209 antigen isoform 3) c4(56408, 56409) c5(56410, 56411, 56412, 56413, 56414, 56415) c6( ) c7(bM, Dw, fR, jx, al, fr, aBl, q, aFM, AW, F, kH, h, Ds, wc, hl, ge, r, lo, aDm, hp, yq, aCK, eE, iV, iQ, Jr, ig, AH, y); #c1(CD209) c2(NP_001138369) c3(CD209 antigen isoform 4) c4(56416, 56417, 56418) c5(56419, 56420, 56421, 56422, 56423, 56424, 56425, 56426, 56427, 56428, 56429) c6( ) c7(bM, Dw, fR, jx, al, fr, aBl, q, aFM, AW, F, kH, h, Ds, wc, hl, ge, r, lo, aDm, hp, yq, aCK, eE, iV, il, Jr, ig, AH, y); #c1 (CD209) c2(NP_001138371) c3(CD209 antigen isoform 8) c4( ) c5(56430, 56431, 56432, 56433, 56434) c6( ) c7(bM, Dw, fR, jx, al, fr, aBl, q, aFM, AW, F, kH, h, Ds, wc, hl, ge, r, lo, aDm, hp, yq, aCK, eE, iV, iQ, Jr, ig, AH, y); #c1 (CD209) c2(NP_066978) c3(CD209 antigen isoform 1) c4(207951) c5(207952, 207953, 207954, 207955, 207956, 207957, 207958) c6( ) c7(bM, Dw, fR, jx, al, fr, aBl, q, aFM, AW, F, kH, h, Ds, wc, hl, ge, r, lo, aDm, hp, yq, aCK, eE, iV, iQ, Jr, ig, AH, y); #c1(CD22) c2(NP_001172028) c3(B-cell receptor CD22 isoform 2 precursor) c4(72277) c5(72278, 72279, 72280, 72281, 72282, 72283) c6( ) c7(T, fM, iW, jh, fd, C, hF, ai, zn, x, b, fx, oG, ql, eZ, pb, ck, ou, iT, cj, uo, XO, k, i, bt); #c1(CD22) c2(NP_001172029) c3(B-cell receptor CD22 isoform 3 precursor) c4(72284) c5(72285, 72286, 72287, 72288, 72289, 72290) c6( ) c7(T, fM, iW, jh, fd, C, hF, ai, zn, x, b, fX, OG, ql, eZ, pb, ck, ou, iT, cj, uo, XO, k, i, bt); #c1(CD22) c2(NP_001172030) c3(B-cell receptor CD22 isoform 4 precursor) c4(72291) c5(72292, 72293, 72294, 72295, 72296, 72297) c6( ) c7(T, fM, iW, jh, fd, C, hF, ai, zn, x, b, fx, OG, ql, eZ, pb, ck, ou, iT, cj, uo, XO, k, i, bt); #c1(CD22) c2(NP_001265346) c3(B-cell receptor CD22 isoform 5) c4(98346) c5(98347, 98348, 98349, 98350, 98351, 98352) c6( ) c7(T, fM, iW, jh, fd, C, hF, ai, zn, x, b, fX, OG, ql, eZ, pb, ck, ou, iT, cj, uo, XO, k, i, bt); #c1(CD22) c2(NP_001762) c3(B-cell receptor CD22 isoform 1 precursor) c4(120896, 120897) c5(120898, 120899, 120900, 120901, 120902, 120903, 120904, 120905, 120906, 120907, 120908, 120909, 120910, 120911) c6(120912, 120913) c7(T, fM, iW, jh, fd, C, hF, ai, zn, x, b, fx, OG, ql, eZ, pb, ck, ou, iT, cj, uo, XO, k, i, bt); #c1(CD226) c2(NP_001290547) c3(CD226 antigen isoform a precursor) c4(115513) c5(115514, 115515, 115516, 115517, 115518, 115519) c6( ) c7(bM, T, lo, jh, fd, cl, aoj, ci, V, q, ad, o, dP, yF, OV, A, Y, aFE, cr, r, eZ, ve, pb, afW, y, Pr, B, hv); #c1(CD24) c2(XP_011533677) c3 (signal transducer CD24 isoform X1) c4( ) c5(329740) c6( ) c7(T, kh, dA, jh, aA, n, fr, jE, ci, V, q, ad, er, hX, AK, abZ, eA, ft, av, aE, afb, jz, yt, aT, By, dL, K, asT, ae, eL, iL, ul, li, d, aW, oV, eZ, r, fe, aq, kp, aF, g, as, iR, t, ahv, bz, uC, op, pb, cm, ck, uP, ik, kP, iT, vA, gX, dy, np, m, k, eo, Pr, B); #c1(CD244) c2(NP_001160135) c3(natural killer cell receptor 284 isoform 2 precursor) c4(65105) c5(65106, 65107, 65108, 65109, 65110, 65111) c6( ) c7(ck, Zg, eZ, fl, pb, aaV, y, bt, mC, aR); #c1(CD244) c2(NP_001160136) c3(natural killer cell receptor 284 isoform 3 precursor) c4(65/12) c5(65113, 65114, 65115, 65116, 65117, 65118) c6( ) c7(ck, Zg, eZ, fL, pb, aaV, y, bt, mC, aR); #c1(CD244) c2(NP_057466) c3(natural killer cell receptor 284 isoform 1 precursor) c4(191109) c5(191110, 191111, 191112, 191113, 191114, 191115) c6( ) c7(ck, Zg, eZ, fl, pb, aaV, y, bt, mC, aR); #c1(CD244) c2(XP_011507924) c3(natural killer cell receptor 284 isoform X3) c4(298240) c5(298241, 298242, 298243, 298244, 298245, 298246) c6( ) c7(ck, Zg, eZ, fl, pb, aaV, y, bt, mC, aR); #c1(CD247) c2(NP_000725) c3(T-cell surface glycoprotein CD3 zeta chain isoform 2 precursor) c4(14904) c5(14905, 14906, 14907, 14908, 14909, 14910) c6( ) c7(T, aA, cz, cl, ci, eA, q, mC, o, hX, h, K, aDU, bA, iU, eZ, r, aF, hp, bw, pb, ig, cj, dW, m, fl, y, aFN); #c1(CD247) c2(NP_932170) c3(T-cell surface glycoprotein CD3 zeta chain isoform 1 precursor) c4(258856) c5(258857, 258858, 258859, 258860, 258861, 258862) c6( ) c7(T, aA, cz, cl, ci, eA, q, mC, o, hX, h, K, aDU, bA, iU, eZ, r, aF, hp, bw, pb, ig, cj, dW, m, fl, y, aFN); #c1(CD248) c2(NP_065137) c3(endosialin precursor) c4(204146) c5(204147, 204148, 204149, 204150, 204151, 204152) c6( ) c7(T, ci, ai, ad, wD, fU, ft, afm, ds, cY, awV, d, aFP, MD, Y, eH, eF, kk, cm, aFO, dB, bE, Zn, y); #c1(CD27) c2(NP_001233) c3(CD27 antigen precursor) c4(87760) c5(87761, 87762, 87763, 87764, 87765, 87766) c6( ) c7(aox, bM, T, aov, fM, fR, aFQ, RX, jh, fd, zN, aj, aFR, bX, mC, J, ai, LH, aoH, zn, eA, agd, fx, CJ, pe, x, Oe, ds, aT, h, yu, cy, rB, bc, qw, A, PK, iU, xR, aW, sp, zb, Y, CM, eZ, Of, CF, Og, lo, bz, pb, Wt, Cv, ck, amS, ig, anY, eB, cj, k, aDb, fl, be, y); #c1(CD274) c2(NP_001254635) c3(programmed cell death 1 ligand 1 isoform b precursor) c4(94736) c5(94737, 94738, 94739, 94740, 94741, 94742) c6( ) c7(T, fR, al, fd, ci, bh, aE, hu, hH, eL, bc, cp, aa, JI, aW, m, hl, g, bw, iP, eM, iA, cj, m, ht, fN, n, aj, fr, eW, jz, h, eV, dL, A, mR, lb, fe, aq, yq, jT, cM, bP, eo, fl, aCD, lB, B, fM, jx, eX, aA, V, q, J, eA, cF, lg, aT, hK, ae, aFS, iU, oV, Y, aCH, r, lo, en, t, ck, iM, kP, Uq, fD, bt, Kd, Ke, jh, wV, jE, mC, ad, hX, av, F, oz, ds, hD, amY, d, qZ, je, ff, eZ, dN, aF, es, y, iu); #c1(CD274) c2(NP_054862) c3(programmed cell death 1 ligand 1 isoform a precursor) c4(178126) c5(178127, 178128, 178129, 178130, 178131, 178132) c6( ) c7(T, fR, al, fd, ci, bh, aE, hu, hH, eL, bc, cp, aa, JI, aW, mQ, hl, g, bw, iP, eM, iA, cj, m, ht, fN, n, aj, fr, eW, jz, h, eV, dL, A, mR, lb, fe, aq, yq, jT, cM, bP, eo, fl, aCD, lB, B, fM, jx, eX, aA, V, q, J, eA, cF, lg, aT, hK, ae, aFS, iU, oV, Y, aCH, r, lo, en, t, ck, iM, kP, Uq, fD, bt, Kd, Ke, jh, wV, jE, mC, ad, hX, av, F, oz, ds, hD, amY, d, qZ, je, ff, eZ, dN, aF, es, y, iu); #c1(CD276) c2(NP_079516) c3(CD276 antigen isoform b precursor) c4(218532) c5(218533, 218534, 218535, 218536, 218537, 218538, 218539, 218540, 218541, 218542, 218543, 218544, 218545, 218546, 218547, 218548, 218549, 218550, 218551, 218552, 218553, 218554, 218555, 218556, 218557, 218558, 218559, 218560, 218561, 218562, 218563, 218564, 218565, 218566, 218567, 218568) c6( ) c7(T, qz, if, aw, im, bB, aj, fr, ci, ze, qN, asr, V, q, mC, J, ad, eA, ib, aE, auj, aT, HD, h, Ou, bk, ae, ul, Uf, d, A, aW, Y, ff, eH, aFT, eZ, r, fe, en, aFU, t, eF, cm, pb, Dy, bE, asq, m, fl, fD, y, B, hS); #c1(CD276) c2(XP_011520398) c3(CD276 antigen isoform X1) c4(315255, 315256, 315257, 315258, 315259, 315260, 315261, 315262) c5(315263, 315264, 315265, 315266, 315267, 315268, 315269, 315270, 315271, 315272, 315273, 315274, 315275, 315276, 315277, 315278, 315279, 315280) c6( ) c7(T, qz, if, aw, im, bB, aj, fr, ci, ze, qN, asr, V, q, mC, J, ad, eA, ib, aE, auj, aT, hD, h, Ou, bK, ae, ul, Uf, d, A, aW, Y, ff, eH, aFT, eZ, r, fe, en, aFU, t, eF, cm, pb, Dy, bE, asq, m, fl, fD, y, B, hS); #c1(CD28) c2(NP_001230007) c3(T-cell-specific surface glycoprotein CD28 isoform 3 precursor) c4(85766) c5(85767, 85768, 85769, 85770, 85771, 85772) c6( ) c7(bM, T, if, fd, aFV, wa, x, AW, XE, Ds, K, hH, bc, axu, g, aFU, eE, eM, uy, cj, NI, k, abh, aw, aj, aFF, uE, qN, ai, Ne, h, aFX, A, mR, anP, oo, CM, kk, jT, W, bm, at, aEG, bk, fL, cn, bL, Dw, DM, FM, dA, DD, jx, j, V, pd, q, J, mP, lg, rS, aT, yu, Oi, qw, hM, xR, oV, Y, O, aFT, Pg, r, lo, zM, pb, ck, cT, cl, yW, Zg, hr, wR, gx, Kd, Ke, iW, axd, jh, auq, oH, fu, mC, wD, aFW, hX, fX, ds, hD, ji, lQ, yF, cr, eZ, hp, BG, iV, Bv, z, y, hv); #c1(CD28) c2(NP_006130) c3(T-cell-specific surface glycoprotein CD28 isoform 1 precursor) c4(162289) c5(162290, 162291, 162292, 162293, 162294, 162295) c6( ) c7(bM, T, if, fd, aFV, wa, x, AW, xE, Ds, K, hH, bc, axu, g, aFU, eE, eM, uy, cj, NI, k, abh, aw, aj, aFF, uE, qN, ai, Ne, h, aFX, A, mR, anP, oo, CM, kk, jT, W, bm, at, aEG, bk, fL, cn, bL, Dw, DM, fM, dA, DD, jx, j, V, pd, q, J, mP, lg, rS, aT, yu, Oi, qw, hM, xR, oV, Y, O, aFT, Pg, r, lo, zM, pb, ck, cT, cl, yW, Zg, hr, wR, gx, Kd, Ke, iW, axd, jh, auq, oH, fu, mC, wD, aFW, hX, fX, ds, hD, ji, lQ, yF, cr, eZ, hp, BG, iV, Bv, z, y, hv); #c1(CD28) c2(XP_011510496) c3(T-cell-specific surface glycoprotein CD28 isoform X1) c4(301016) c5(301017, 301018, 301019, 301020, 301021, 301022) c6( ) c7(bM, T, if, fd, aFV, wa, x, AW, xE, Ds, K, hH, bc, axu, g, aFU, eE, eM, uy, cj, NI, k, abh, aw, aj, aFF, uE, qN, ai, Ne, h, aFX, A, mR, anP, oo, CM, kk, jT, W, bm, at, aEG, bk, fl, cn, bL, Dw, DM, fM, dA, DD, jx, j, V, pd, q, J, mP, lg, rS, aT, yu, Oi, qw, hM, xR, oV, Y, fO, aFT, Pg, r, lo, zM, pb, ck, cT, c1, yW, Zg, hr, wR, gx, Kd, Ke, iW, axd, jh, auq, oH, fu, mC, wD, aFW, hX, fx, ds, hD, ji, lQ, yF, cr, eZ, hp, BG, iV, Bv, z, y, hv); #c1(CD28) c2(XP_011510499) c3(T-cell-specific surface glycoprotein CD28 isoform X4) c4(301023) c5(301024, 301025, 301026, 301027, 301028, 301029) c6( ) c7(bM, T, if, fd, aFV, wa, x, AW, xE, Ds, K, hH, bc, axu, g, aFU, eE, eM, uy, cj, NI, k, abh, aw, aj, aFF, uE, qN, ai, Ne, h, aFX, A, mR, anP, oo, CM, kk, jT, W, bm, at, aEG, bk, fl, cn, bL, Dw, DM, fM, dA, DD, jx, j, V, pd, q, J, mP, lg, rS, aT, yu, Oi, qw, hM, xR, oV, Y, fO, aFT, Pg, r, lo, zM, pb, ck, cT, cl, yW, Zg, hr, wR, gx, Kd, Ke, iW, axd, jh, auq, oH, fu, mC, wD, aFW, hX, fx, ds, hD, ji, lQ, yF, cr, eZ, hp, BG, iV, Bv, z, y, hv); #c1(CD2AP) c2(NP_036252) c3(CD2-associated protein) c4(172655) c5(172656, 172657, 172658, 172659, 172660, 172661) c6( ) c7(fM, eR, aFY, aj, ah, aY, aFZ, J, aGa, no, oj, vw, lG, af, A, aGb, cH, gi, f, sk, xA, vW, xj, hc, U, dy); #c1(CD300A) c2(NP_001243770) c3(CMRF35-like molecule 8 isoform 2 precursor) c4(91688, 91689) c5(91690, 91691, 91692, 91693, 91694, 91695) c6( ) c7(lo, aw, jx); #c1(CD300A) c2(NP_009192) c3(CMRF35-like molecule 8 isoform 1 precursor) c4(171416) c5(171417, 171418, 171419, 171420, 171421, 171422) c6( ) c7(lo, aw, jx); #c1(CD300A) c2(XP_005257049) c3(CMRF35-like molecule 8 isoform X3) c4( ) c5(274640, 274641, 274642, 274643, 274644, 274645) c6( ) c7(lo, aw, jx); #c1(CD300A) c2(XP_011522556) c3(CMRF35-like molecule 8 isoform X4) c4(317138) c5(317139, 317140, 317141, 317142, 317143, 317144) c6( ) c7(lo, aw, jx); #c1(CD300C) c2(NP_006669) c3(CMRF35-like molecule 6 precursor) c4(167013) c5(167014, 167015, 167016, 167017, 167018, 167019) c6( ) c7(T); #c1(CD300LF) c2(NP_001276011) c3(CMRF35-like molecule 1 isoform 2) c4(107736) c5(107737, 107738, 107739, 107740, 107741) c6( ) c7(Nq, Nr, jx, lo, Xm, DF); #c1(CD300LF) c2(NP_001276012) c3(CMRF35-like molecule 1 isoform 3 precursor) c4(107742) c5(107743, 107744, 107745, 107746, 107747, 107748) c6( ) c7(Nq, Nr, jx, lo, Xm, DF); #c1(CD300LF) c2(NP_001276014) c3(CMRF35-like molecule 1 isoform 5) c4( ) c5(107749) c6( ) c7(Nq, Nr, jx, lo, Xm, DF); #c1(CD300LF) c2(NP_001276015) c3(CMRF35-like molecule 1 isoform 6) c4(107750) c5(107751, 107752, 107753, 107754, 107755) c6( ) c7(Nq, Nr, jx, lo, Xm, DF); #c1(CD300LF) c2(NP_620587) c3(CMRF35-like molecule 1 isoform 1 precursor) c4(237818) c5(237819, 237820, 237821, 237822, 237823, 237824) c6( ) c7(Nq, Nr, jx, lo, Xm, DF); #c1(CD300LF) c2(XP_011522677) c3(CMRF35-like molecule 1 isoform X7) c4( ) c5(317223) c6( ) c7(Nq, Nr, jx, lo, Xm, DF); #c1(CD302) c2(NP_001185692) c3(CD302 antigen isoform 2 precursor) c4(76059) c5(76060, 76061, 76062, 76063, 76064, 76065) c6( ) c7(eV, ae, eX, ci); #c1(CD302) c2(NP_055695) c3(CD302 antigen isoform 1 precursor) c4(182829) c5(182830, 182831, 182832, 182833, 182834, 182835) c6( ) c7(eV, ae, eX, ci); #c1(CD320) c2(NP_001159367) c3(CD320 antigen isoform 2 precursor) c4(63876) c5(63877, 63878, 63879, 63880, 63881, 63882, 63883, 63884, 63885, 63886) c6( ) c7(aGc, Lk, mN, Ll, bu); #c1(CD320) c2(NP_057663) c3(CD320 antigen isoform 1 precursor) c4(192208, 192209) c5(192210, 192211, 192212, 192213, 192214, 192215, 192216, 192217) c6( ) c7(aGc, Lk, mN, LI, bu); #c1(CD33) c2(NP_001076087) c3(myeloid cell surface antigen CD33 isoform 2 precursor) c4(37377) c5(37378, 37379, 37380, 37381, 37382, 37383, 37384, 37385, 37386, 37387, 37388, 37389) c6( ) c7(T, os, fM, FR, jh, bs, oZ, v, hF, br, x, aT, h, hn, K, ox, jj, H, af, iL, iU, hG, aW, ff, en, iV, Fu, bw, ck, ou, eM, iT, dc, cj, PV, k, bt, L); #c1(CD33) c2(NP_001171079) c3(myeloid cell surface antigen CD33 isoform 3 precursor) c4(69811) c5(69812, 69813, 69814, 69815, 69816, 69817, 69818, 69819, 69820, 69821, 69822) c6( ) c7(T, os, fM, fR, jh, bs, oZ, v, hF, br, x, aT, h, hn, K, ox, jj, H, af, iL, iU, hG, aW, ff, en, iV, Fu, bw, ck, ou, eM, iT, dc, cj, PV, k, bt, L); #c1(CD33) c2(XP_011525833) c3(myeloid cell surface antigen CD33 isoform X2) c4(320236, 320237, 320238, 320239) c5(320240, 320241) c6( ) c7(T, os, fM, fR, jh, bs, oZ, v, hF, br, x, aT, h, hn, K, ox, jj, H, af, iL, iU, hG, aW, ff, en, iV, Fu, bw, ck, ou, eM, iT, dc, cj, PV, k, bt, L); #c1(CD33) c2(XP_011525834) c3(myeloid cell surface antigen CD33 isoform X1) c4( ) c5(320242, 320243, 320244, 320245, 320246) c6( ) c7(T, os, fM, fR, jh, bs, oZ, v, hF, br, x, aT, h, hn, K, ox, jj, H, af, iL, iU, hG, aW, ff, en, iV, Fu, bw, ck, ou, eM, iT, dc, cj, PV, k, bt, L); #c1(CD34) c2(NP_001020280) c3(hematopoietic progenitor cell antigen CD34 isoform a precursor) c4(28546, 28547, 28548, 28549) c5(28550, 28551, 28552, 28553, 28554, 28555, 28556, 28557, 28558, 28559, 28560, 28561, 28562, 28563, 28564, 28565, 28566, 28567, 28568, 28569, 28570, 28571, 28572, 28573) c6( ) c7(bM, T, os, aC, Js, ea, if, e, qx, al, fd, gf, ci, Ap, ip, x, xy, aGm, aE, OG, Co, SV, dV, DO, K, hu, DY, dF, ln, li, yo, aW, Ls, hl, KX, g, cm, bw, ajH, eM, nB, iT, vA, aGe, mx, cj, dW, k, m, pw, ym, in, Cx, dD, xu, vl, aw, ht, n, aj, fr, qN, eh, eW, br, Bl, no, KH, Ne, h, By, hn, eV, ox, dx, cY, afu, Lg, XE, Cu, Lh, aGd, yq, pm, mo, jT, apX, op, yR, vU, Of, hk, kz, Vy, ajd, pg, aEG, Lq, eo, ajW, agy, B, Jo, aAE, aGk, aGn, dA, jx, DV, ex, fZ, aA, hz, afK, hy, j, v, V, q, J, er, eA, mP, jZ, aT, cy, dz, rO, jj, qk, af, sb, arl, s, vT, iU, oV, Y, aGf, r, lo, en, t, f, ck, Ta, BU, iM, ac, QG, aGo, aGi, RW, hr, oX, zu, bt, id, ow, qb, iW, oA, bs, jh, oZ, OH, aGl, eq, hF, iJ, hX, Jm, hd, fx, CJ, ib, Ot, oW, LA, HD, iY, aGh, fb, nJ, pq, iL, d, BN, ue, et, cW, qt, afe, je, ff, nK, eZ, gl, Ej, xv, aF, BG, jg, iV, aGj, bz, Hp, LB, age, pc, kg, yE, z, eB, auH, aCl, CO, aS, aGg, i, y, fh, ne, jr, Cp, Tc); #c1(CD34) c2(NP_001764) c3(hematopoietic progenitor cell antigen CD34 isoform b precursor) c4(120914) c5(120915, 120916, 120917, (20918, 120919, 120920) c6( ) c7(bM, T, os, aC, Js, ea, if, e, qx, al, fd, gf, ci, Ap, ip, x, xy, aGm, aE, OG, Co, SV, dV, DO, K hu, DY, dF, ln, li, yo, aW, Ls, hl, KX, g, cm, bw, ajH, eM, nB, lT, vA, aGe, mx, cj, dW, k, m, pw, ym, in, Cx, dD, xu, vl, aw, ht, n, aj, fr, qN, eh, eW, br, Bl, no, kH, Ne, h, By, hn, eV, ox, dx, cY, afu, Lg, XE, Cu, Lh, aGd, yq, pm, mo, jT, apX, op, yR, vU, Qf, hk, kz, Vy, ajd, pg, aEG, Lq, eo, ajW, agy, B, Jo, aAE, aGk, aGn, dA, jx, DV, ex, fZ, aA, hz, afK, hy, j, v, V, q, J, er, eA, mP, jZ, aT, cy, dz, rO, jj, qk, af, sb, arl, s, vT, iU, oV, Y, aGf, r, lo, en, t, f, ck, Ta, BU, iM, ac, QG, aGo, aGi, RW, hr, oX, zu, bt, id, ow, qb, iW, oA, bs, jh, oZ, OH, aGl, eq, hF, iJ, hX, Jm, hd, fx, CJ, ib, Ot, oW, LA, hD, iY, aGh, fb, nJ, pq, iL, d, BN, ue, et, cW, qt, afe, je, ff, nK, eZ, gl, Ej, xv, aF, BG, jg, iV, aGj, bz, Hp, LB, age, pc, kg, yE, z, eB, auH, aCI, CO, aS, aGg, i, y, fh, ne, jr, Cp, Tc); #c1(CD36) c2(NP_001001547) c3(platelet glycoprotein 4 isoform 1) c4(18631, 18632, 18633, 18634) c5(18635, 18636, 18637, 18638, 18639, 18640, 18641, 18642, 18643, 18644, 18645, 18646, 18647, 18648, 18649, 18650, 18651, 18652, 18653, 18654, 18655, 18656, 18657, 18658, 18659, 18660, 18661, 18662, 18663, 18664, 18665, 18666, 18667, 18668, 18669) c6( ) c7(T, fR, ea, e, al, bB, axk, dV, ta, K, dQ, aW, fc, hl, eE, eF, bw, mx, vy, nw, eg, aGv, Ro, dD, xu, aw, aj, afE, ct, eh, cd, br, rc, cc, h, lG, DU, ey, ZN, Um, A, fJ, aq, aGq, pm, acD, bJ, W, Fi, va, B, tq, bL, eR, fG, oe, aGt, aGu, cz, v, j, nS, q, J, aT, kf, bK, bq, aFm, af, kl, oV, Y, aGp, r, en, ck, aGs, ac, aGr, mf, dy, bt, qb, axd, bS, dG, eq, dP, F, Dq, oW, vc, iY, cH, PE, cW, ff, dq, Z, qa, LB, bE, ll, gZ, dX, cP, ei, y, xg, gq); #c1(CD36) c2(NP_001276837) c3(platelet glycoprotein 4 isoform 2) c4(108394, 108395, 108396 108397, 108398, 108399) c5(108400, 108401, 108402, 108403, 108404, 108405, 108406, 108407, 108408, 108409, 108410, 108411, 108412, 108413, 108414, 108415, 108416, 108417) c6( ) c7(T, fR, ea, e, al, bB, axk, dV, ta, K, dQ, aW, fc, hl, BE, eF, bw, mx, vy, nw, eg, aGv, Ro, dD, xu, aw, aj, afE, ct, eh, cd, br, rc, cc, h, lG, DU, ey, ZN, Um, A, fJ, aq, aGq, pm, acD, bJ, W, Fi, va, B, tq, bL, eR, fG, oe, aGt, aGu, cz, v, j, nS, q, J, aT, kf, bK, bq, aFm, af, kl, oV, Y, aGp, r, en, ck, aGs, ac, aGr, mf, dy, bt, qb, axd, bS, dG, eq, dP, F, Dq, oW, vc, iY, cH, PE, cW, ff, dq, Z, qa, LB, bE, ll, gZ, dX, cP, ei, y, xg, gq); #c1(CD36) c2(NP_001276838) c3(platelet glycoprotein 4 isoform 3) c4(108418) c5(108419, 108420, 108421, 108422, 108423, 108424, 108425, 108426, 108427, 108428, 108429, 108430, 108431, 108432, 108433, 108434, 108435, 108436, 108437, 108438, 108439, 108440, 108441, 108442, 108443, 108444, 108445, 108446, 108447, 108448, 108449, 108450, 108451) c6( ) c7(T, fR, ea, e, al, bB, axk, dV, ta, K, dQ, aW, fc, hl, eE, eF, bw, mx, vy, nw, eg, aGv, Ro, dD, xu, aw, aj, afE, ct, eh, cd, br, rc, cc, h, lG, DU, ey, ZN, Um, A, fJ, aq, aGq, pm, acD, bJ, W, Fi, va, B, tq, bL, eR, fG, oe, aGt, aGu, cz, v, j, nS, q, J, aT, kf, bK, bq, aFm, af, kl, oV, Y, aGp, r, en, ck, aGs, ac, aGr, mf, dy, bt, qb, axd, bS, dG, eq, dP, F, Dq, oW, vc, iY, cH, PE, cW, ff, dq, Z, qa, LB, bE, ll, gZ, dX, cP, ei, y, xg, gq); #c1(CD36) c2(NP_001276840) c3(platelet glycoprotein 4 isoform 4) c4(108458) c5(108459, 108460, 108461, 108462, 108463, 108464) c6( ) c7(T, fR, ea, e, al, bB, axk, dV, ta, K, dQ, aW, fc, hl, eE, eF, bw, mx, vy, nw, eg, aGv, Ro, dD, xu, aw, aj, afE, ct, eh, cd, br, rc, cc, h, lG, DU, ey, ZN, Um, A, fJ, aq, aGq, pm, acD, bJ, W, Fi, va, B, tq, bL, eR, fG, oe, aGt, aGu, cz, v, j, nS, q, J, aT, kf, bK, bq, aFm, af, kl, oV, Y, aGp, r, en, ck, aGs, ac, aGr, mf, dy, bt, qb, axd, bS, dG, eq, dP, F, Dq, oW, vc, iY, cH, PE, cW, ff, dq, Z, qa, LB, bE, ll, gZ, dX, cP, ei, y, xg, gq); #c1(CD37) c2(NP_001765) c3(leukocyte antigen CD37 isoform A) c4(120921, 120922, 120923) c5(120924, 120925, 120926, 120927, 120928, 120929, 120930, 120931, 120932, 120933, 120934, 120935, 120936, 120937, 120938, 120939, 120940, 120941) c6( ) c7(ck, x, T, ds, iW, cj, ey, k, rm); #c1(CD37) c2(XP_005259492) c3(leukocyte antigen CD37 isoform X2) c4(276113) c5( ) c6( ) c7(ck, x, T, ds, iW, cj, ey, k, rm); #c1(CD37) c2(XP_011525846) c3(leukocyte antigen CD37 isoform X1) c4( ) c5(320258, 320259, 320260, 320261, 320262, 320263) c6( ) c7(ck, x, T, ds, iW, cj, ey, k, rm); #c1(CD38) c2(NP_001766) c3(ADP-ribosyl cyclase/cyclic ADP-ribose hydrolase 1) c4(120942, 120943) c5(120944, 120945, 120946, 120947, 120948, 120949, 120950, 120951, 120952, 120953, 120954, 120955) c6( ) c7(bL, bM, T, fM, dA, fR, qb, aDT, oA, fZ, bs, aGw, aj, v, j, fd, jh, hF, br, J, x, CJ, fX, cq, nX, oW, kH, Ne, h, hn, j, rz, pk, amO, af, bA, sj, A, asp, qZ, iU, je, oV, Pg, eZ, fQ, dq, hp, aom, f, cm, bz, GR, bw, DF, Wt, Cv, zJ, ck, bN, ar, iM, dJ, cj, k, fl, ei, hh, y, bt, fh, agZ); #c1(CD3D) c2(NP_000723) c3(T-cell surface glycoprotein CD3 delta chain isoform A precursor) c4(14883) c5(14884, 14885, 14886, 14887, 14888, 14889) c6( ) c7(ck, T, aGz, dA, iq, fJ, CM, hl, jh, aGy, aGx, dW, hp, anx, aDU, bc, aGA); #c1(CD3D) c2(NP_001035741) c3(T-cell surface glycoprotein CD3 delta chain isoform B precursor) c4(33024) c5(33025, 33026, 33027, 33028, 33029, 33030) c6( ) c7(ck, T, aGz, dA, iq, fJ, CM, hl, jh, aGy, aGx, dW, hp, anx, aDU, bc, aGA); #c1(CD3E) c2(NP_000724) c3(T-cell surface glycoprotein CD3 epsilon chain precursor) c4(14890, 14891) c5(14892, 14893, 14894, 14895, 14896, 14897, 14898, 14899, 14900, 14901, 14902, 14903) c6( ) c7(aGB, id, dA, aGC, aGx, fr, ci, hF, cd, ad, aGA, azq, ae, A, je, oV, Y, hp, g, Z, sS, iM, dW, m, y); #c1(CD3EAP) c2(NP_036231) c3(DNA-directed RNA polymerase 1 subunit RPA34 isoform 2) c4(172498) c5(172499, 172500, 172501, 172502, 172503, 172504, 172505, 172506, 172507) c6( ) c7(Kd, dD, ea, jh, cz, fd, eq, Qo, ai, dL, ji, an, akX, s, Fm, Y, r, bz, sS, VJ, eM, wB); #c1(CD3G) c2(NP_000064) c3(T-cell surface glycoprotein CD3 gamma chain precursor) c4(1096, 1097) c5(1098, 1099, 1100, 1101, 1102, 1103, 1104, 1105, 1106, 1107, 1108, 1109) c6( ) c7(ck, aGC, dA, CM, jh, h, dW, hp, ey, aGD, y, bt); #c1(CD4) c2(NP_000607) c3(T-cell surface glycoprotein CD4 isoform 1 precursor) c4(13694) c5(13695, 13696, 13697, 13698, 13699, 13700, 13701, 13702, 13703, 13704, 13705, 13706, 13707) c6(13708, 13709) c7(bL, FR, jx, fN, fd, hy, qj, V, HC, aT, h, dL, ey, Nr, aGF, BN, A, gT, ff, eZ, uY, lo, po, aoD, en, Nq, ar, eU, iQ, akx, bE, aGE, at, aDE, hE, ne, y); #c1(CD40) c2(NP_001241) c3(tumor necrosis factor receptor superfamily member 5 isoform 1 precursor) c4(89202) c5( ) c6( ) c7(bM, T, fR, pf, ea, e, abM, fd, bB, cL, zh, ci, aY, x, AW, xy, xE, aE, aDk, M, Ds, K, zy, dQ, hH, eL, bc, pk, cp, eG, aW, axu, az, hl, HA, aGQ, g, as, eE, cm, bw, aGl, gt, nB, eM, iA, mx, U, cj, zl, aGN, k, m, Uw, fy, dD, aik, aw, fN, n, aj, fr, uE, ai, eW, Oe, aGR, jz, no, cU, lG, By, hn, dL, h, eV, he, aGL, A, Cw, zb, gi, aGG, aGM, agb, aga, fe, Og, xe, cM, Wt, asE, KT, bP, aGH, w, aGO, Lp, at, Pr, B, cB, aox, bL, Dw, fM, dA, eR, wL, dk, eX, rf, aA, fF, ti, hz, v, j, aGK, V, eA, J, eJ, zn, ady, oT, mP, aT, Oi, cy, bK, ae, aH, af, bA, eu, qw, hM, aGP, iU, xR, oV, Y, en, t, pb, aFA, ck, rR, OW, ac, agc, wg, of, hr, fP, auT, aor, oX, fD, UF, bt, Ze, ZA, id, iW, jh, eq, jE, ad, hX, dP, av, abk, hd, aop, agd, LA, HD, NZ, lQ, ji, MG, lx, cH, cW, Gv, ff, aFE, Of, cr, eZ, gl, aF, hp, jg, iV, bz, aGJ, eK, LB, Bv, age, eU, zr, amS, bE, do, wz, aS, ei, lf, X, y); #c1(CD40) c2(NP_690593) c3(tumor necrosis factor receptor superfamily member 5 isoform 2 precursor) c4(244871) c5(244872, 244873, 244874, 244875, 244876, 244877) c6( ) c7(bM, T, fR, pf, ea, e, abM, fd, bB, cL, zh, ci, aY, x, AW, xy, xE, aE, aDk, M, Ds, K, zy, dQ, hH, eL, bc, pk, cp, eG, aW, axu, az, hl, HA, aGQ, g, as, eE, cm, bw, aGl, gt, nB, eM, iA, mx, U, cj, zl, aGN, k, m, Uw, fy, dD, aik, aw, fN, n, aj, fr, uE, ai, eW, Oe, aGR, jz, no, cU, lG, By, hn, dL, h, eV, he, aGL, A, Cw, zb, gi, aGG, aGM, agb, aga, fe, Og, xe, cM, Wt, asE, kT, bP, aGH, w, aGO, Lp, at, Pr, B, cB, aox, bL, Dw, fM, dA, eR, wL, dk, eX, rf, aA, fF, ti, hz, v, j, aGK, V, eA, J, eJ, zn, ady, oT, mP, aT, Oi, cy, bK, ae, aH, af, bA, eu, qw, hM, aGP, iU, xR, oV, Y, en, t, pb, aFA, ck, rR, OW, ac, agc, wg, of, hr, fP, auT, aor, oX, fD, UF, bt, Ze, ZA, id, iW, jh, eq, jE, ad, hX, dP, av, abk, hd, aop, agd, LA, HD, NZ, lQ, ji, MG, lx, cH, cW, Gv, ff, aFE, Of, cr, eZ, gl, af, hp, jg, iV, bz, aGJ, eK, LB, Bv, age, eU, zr, amS, bE, d, wz, aS, ei, lf, X, y); #c1(CD40) c2(XP_011527411) c3(tumor necrosis factor receptor superfamily member 5 isoform X3) c4( ) c5(322142, 322143) c6( ) c7(bM, T, fR, pf, ea, e, abM, fd, bB, cL, zh, ci, aY, x, AW, xy, xE, aE, aDk, M, Ds, K, zy, de, hH, eL, bc, pk, cp, eG, aW, axu, az, hl, HA, aGQ, g, as, eE, cm, bw, aGl, gt, nB, eM, iA, mx, U, cj, zl, aGN, k, m, Uw, fy, dD, aik, aw, fN, n, aj, fr, uE, ai, eW, Oe, aGR, jz, no, CU, lG, By, hn, dL, h, eV, he, aGL, A, Cw, zb, gi, aGG, aGM, agb, aga, fe, Og, xe, cM, Wt, asE, kT, bP, aGH, w, aGO, Lp, at, Pr, B, cB, aox, bL, Dw, fM, dA, eR, wL, dk, eX, rf, aA, fF, ti, hz, v, j, aGK, V, eA, J, eJ, zn, ady, oT, mP, aT, Oi, cy, bK, ae, aH, af, bA, eu, qw, hM, aGP, iU, xR, oV, Y, en, t, pb, aFA, ck, rR, OW, ac, agc, wg, of, hr, fP, auT, aor, oX, fD, UF, bt, Ze, zA, id, iW, jh, eq, jE, ad, hX, dP, av, abk, hd, aop, agd, LA, HD, NZ, lQ, ji, MG, lx, cH, cW, Gv, ff, aFE, Of, cr, eZ, gl, aF, hp, jg, iV, bz, aGJ, ek, LB, Bv, age, eU, zr, amS, bE, dO, wz, aS, ei, lf, X, y); #c1(CD40LG) c2(NP_000065) c3(CD40 ligand) c4(1110, lift) c5(1112, 1113, 1114, 1115, 1116, 1117, 1118, 1119, 1120, 1121, 1122, 1123) c6( ) c7(oO, bM, T, lD, ea, e, al, fd, bB, zh, ci, bh, aGX, x, AW, aE, il, dV, Ds, DY, pk, eL, bc, eN, hm, Ok, ly, DA, aW, axu, md, hl, aGQ, aw, g, aFU, ve, cm, bw, aGl, eM, nB, mx, aiM, cj, U, zl, dW, k, m, R, eg, iu, Ro, dD, aw, n, aj, aGU, fr, uE, eh, zo, ai, Oe, ft, jz, kH, h, lG, jy, dL, eV, A, Cw, zb, ask, fe, aq, jT, xe, lx, Wt, asE, aGH, dB, lf, eo, fL, B, bL, Dw, fM, dA, eR, wL, eX, aA, fF, v, j, C, ze, aGK, abC, V, q, J, aGT, eA, ady, mP, rS, aT, Oi, zg, ae, af, eu, kl, aGP, xR, oV, Y, kX, bg, lC, r, awu, en, aoG, t, pb, lY, aFA, ck, ac, c, hc, hr, aGV, wR, dy, fD, bt, Ze, ZA, ao, hS, gV, id, iW, axd, jh, dG, eq, hF, mC, aGW, ad, av, abk, hd, ib, tT, ds, lC, NZ, lQ, vl, d, ue, hG, ff, aFE, aGS, cr, eZ, dN, aF, jg, iV, bz, dr, Bv, eU, amS, zr, bE, dO, wz, aS, ME, bC, ei, zk, X, y); #c1(CD44) c2(NP_000601) c3(CD44 antigen isoform 1 precursor) c4(13671) c5(13672, 13673, 13674, 13675, 13676, 13677) c6( ) c7(bM, T, aC, UH, Tb, fR, Zz, ea, if, e, fd, bB, qj, ci, lR, ip, x, afr, aE, il, rJ, pX, aeO, ajv, K, DY, H, pk, eL, aiJ, cp, aa, eG, yo, aeR, dH, JI, aW, Ls, adw, XV, fc, nY, g, as, pl, hJ, eF, cm, aHd, aAW, ale, lt, gt, nB, avZ, AV, eM, iA, iT, cj, k, m, iu, dD, vl, ajm, aEj, wu, sw, zX, QO, NT, fg, n, aj, kv, fr, qN, zP, cd, SW, ai, Bl, ft, jz, gd, kH, h, lG, By, hn, dL, ey, SH, hB, Ha, pl, yi, aiB, PX, aGZ, AQ, ahJ, aq, fe, Pp, kp, Hu, Ca, cM, Qy, bP, dB, hk, anZ, vb, hL, bm, E, fL, bD, it, B, bL, fM, dA, Dh, fG, yk, jx, aA, hy, v, j, aHa, bX, acn, aHc, V, pd, q, J, er, eA, eJ, Zp, aEE, mP, Vr, qK, HC, aHb, aT, j, Fg, ae, ne, af, bA, kl, s, vT, nO, oV, Y, ev, fO, iU, iZ, r, en, cl, t, ik, pb, ck, sS, nM, kP, Dy, ac, iM, nG, aGY, gX, wR, fD, kZ, bt, rA, gx, Gl, nZ, id, Mb, qz, iW, jN, jh, Fb, Gn, eq, EA, Fc, mC, ad, afb, aoH, abZ, dP, hX, hd, ib, av, Tg, db, NS, akJ, LA, io, hP, lQ, wm, fb, Yi, d, BN, TY, je, ff, eH, eZ, aF, VI, iV, iR, bz, LB, es, bF, apM, Ew, zr, bE, AR, Hs, cP, adg, Pk, X, y); #c1(CD44) c2(NP_001001389) c3(CD44 antigen isoform 2 precursor) c4(18213) c5(18214, 18215, 18216, 18217, 18218, 18219) c6( ) c7(bM, T, aC, UH, Tb, FR, Zz, ea, if, e, fd, bB, qj, ci, lR, ip, x, afr, aE, il, rJ, pX, aeI, ajv, K, DY, H, pk, eL, aiJ, cp, aa, eG, yo, aeR, dH, JI, aW, Ls, adw, XV, fc, nY, g, as, pl, hJ, eF, cm, aHd, aAW, ale, lt, gt, nB, avZ, AV, eM, iA, iT, cj, k, m, iu, dD, vl, ajm, aEj, wu, sw, zX, oO, NT, fg, n, aj, kv, fr, qN, zP, cd, SW, ai, Bl, ft, jz, gd, KH, h, lG, By, hn, dL, ey, SH, hB, Ha, pl, yi, aiB, PX, aGZ, AQ, ahJ, aq, fe, Pp, kp, Hu, Ca, CM, Qy, bP, dB, hk, anZ, vb, hL, bm, E, fL, bD, it, B, bL, fM, dA, Dh, fG, yk, jx, aA, hy, v, j, aHa, bX, acn, aHc, V, pd, q, J, er, eA, eJ, Zp, aEE, mP, Vr, qK, HC, aHb, aT, j, Fg, ae, ne, af, bA, kl, s, vT, nO, oV, Y, ev, fO, iU, iZ, r, en, cl, t, ik, pb, ck, sS, nM, kP, Dy, ac, iM, nG, aGY, gX, wR, fD, kZ, bt, rA, gx, GI, nZ, id, Mb, qz, iW, jN, jh, Fb, Gn, eq, EA, Fc, mC, ad, afb, aoH, abZ, dP, hX, hd, ib, av, Tg, db, NS, akJ, LA, io, hP, lQ, wm, fb, Yi, d, BN, TY, je, ff, eH, eZ, aF, VI, iV, iR, bz, LB, es, bF, apM, Ew, zr, bE, AR, Hs, cP, adg, Pk, X, y); #c1(CD44) c2(NP_001001390) c3(CD44 antigen isoform 3 precursor) c4(18220) c5(18221, 18222, 18223, 18224, 18225, 18226) c6( ) c7(bM, T, aC, UH, Tb, fR, Zz, ea, if, e, fd, bB, qj, ci, lR, ip, x, afr, aE, il, rJ, pX, aeO, ajv, K, DY, H, pk, eL, aiJ, cp, aa, eG, yo, aeR, dH, JI, aW, Ls, adw, XV, fc, nY, g, as, pl, hJ, eF, cm, aHd, aAW, ale, lt, gt, nB, avZ, AV, eM, iA, iT, cj, k, m, iu, dD, vl, ajm, aEj, wu, sw, zX, QO, NT, fg, n, aj, kv, fr, qN, zP, cd, SW, ai, Bl, ft, jz, gd, KH, h, lG, By, hn, dL, ey, SH, hB, Ha, pl, yi, aiB, PX, aGZ, AQ, ahJ, aq, fe, Pp, kp, Hu, Ca, CM, Qy, bP, dB, hk, anZ, vb, hL, bm, E, fL, bD, it, B, bL, fM, dA, Dh, fG, yk, jx, aA, hy, v, j, aHa, bX, acn, aHc, V, pd, q, J, er, eA, eJ, Zp, aEE, mP, Vr, qK, HC, aHb, aT, j, Fg, ae, ne, af, bA, kl, s, vT, nO, oV, Y, ev, fO, iU, iZ, r, en, cl, t, ik, pb, ck, sS, nM, kP, Dy, ac, iM, nG, aGY, gX, wR, fD, kZ, bt, rA, gx, Gl, nZ, id, Mb, qz, iW, jN, jh, Fb, Gn, eq, EA, Fc, mC, ad, afb, aoH, abZ, dP, hX, hd, ib, av, Tg, db, NS, akJ, LA, io, hP, lQ, wm, fb, Yi, d, BN, TY, je, ff, eH, eZ, aF, VI, iV, iR, bz, LB, es, bF, apM, Ew, zr, bE, AR, Hs, cP, adg, Pk, X, y); #c1(CD44) c2(NP_001001391) c3(CD44 antigen isoform 4 precursor) c4(18227, 18228) c5(18229, 18230, 18231, 18232, 18233, 18234, 18235) c6( ) c7(bM, T, aC, UH, Tb, fR, Zz, ea, if, e, fd, bB, qj, ci, lR, ip, x, afr, aE, il, rJ, pX, aeI, ajv, K, DY, H, pk, eL, aiJ, cp, aa, eG, yo, aeR, dH, JI, aW, Ls, adw, XV, fc, nY, g, as, pl, hJ, eF, cm, aHd, aAW, aHe, lt, gt, nB, avZ, AV, eM, iA, iT, cj, k, m, iu, dD, vl, ajm, aEj, wu, sw, zX, QO, NT, fg, n, aj, kv, fr, qN, zP, cd, SW, ai, Bl, ft, jz, gd, kH, h, lG, By, hn, dL, ey, SH, hB, Ha, pl, yi, aiB, PX, aGZ, AQ, ahJ, aq, fe, Pp, kp, Hu, Ca, CM, Qy, bP, dB, hk, anZ, vb, hL, bm, E, fL, bD, it, B, bL, fM, dA, Dh, fG, yk, jx, aA, hy, v, j, aHa, bX, acn, aHc, V, pd, q, J, er, eA, eJ, Zp, aEE, mP, Vr, qK, HC, aHb, aT, j, Fg, ae, ne, af, bA, kl, s, vT, nO, oV, Y, ev, fO, iU, iZ, r, en, cl, t, iK, pb, ck, sS, nM, kP, Dy, ac, iM, nG, aGY, gX, wR, fD, kZ, bt, rA, gx, GI, nZ, id, Mb, qz, iW, jN, jh, Fb, Gn, eq, EA, Fc, mC, ad, afb, aoH, abZ, dP, hX, hd, ib, av, Tg, db, NS, akJ, LA, io, hP, lQ, wm, fb, Yi, d, BN, TY, je, ff, eH, eZ, aF, VI, iV, iR, bz, LB, es, bF, apM, Ew, zr, bE, AR, Hs, cP, adg, Pk, X, y); #c1(CD44) c2(NP_001001392) c3(CD44 antigen isoform 5 precursor) c4(18236) c5(18237, 18238, 18239, 18240, 18241, 18242, 18243, 18244, 18245, 18246, 18247, 18248, 18249, 18250, 18251, 18252, 18253) c6( ) c7(bM, T, aC, UH, Tb, fR, Zz, ea, if, e, fd, bB, qj, ci, lR, ip, x, afr, aE, il, rJ, pX, ae, ajv, K, DY, H, pk, eL, aiJ, cp, aa, eG, yo, aeR, dH, JI, aW, Ls, adw, XV, fc, nY, g, as, pl, hJ, eF, cm, aHd, aAW, aHe, lt, gt, nB, avZ, AV, eM, iA, iT, cj, k, m, iu, dD, vl, ajm, aEj, wu, sw, zX, oO, NT, fg, n, aj, kv, fr, qN, zP, cd, SW, ai, Bl, ft, jz, gd, kH, h, lG, By, hn, dL, ey, SH, hB, Ha, pl, yi, aiB, PX, aGZ, AQ, ahJ, aq, fe, Pp, kp, Hu, Ca, cM, Qy, bP, dB, hk, anZ, vb, hL, bm, E, fL, bD, it, B, bL, fM, dA, Dh, fG, yk, jx, aA, hy, v, j, aHa, bX, acn, aHc, V, pd, q, J, er, eA, eJ, Zp, aEE, mP, Vr, qK, HC, aHb, aT, j, Fg, ae, ne, af, bA, kl, s, vT, nO, oV, Y, ev, fO, iU, iZ, r, en, cl, t, iK, pb, ck, sS, nM, kP, Dy, ac, iM, nG, aGY, gX, wR, fD, kZ, bt, rA, gx, Gl, nZ, id, Mb, qz, iW, jN, jh, Fb, Gn, eq, EA, Fc, mC, ad, afb, aoH, abZ, dP, hX, hd, ib, av, Tg, db, NS, akJ, LA, io, hP, lQ, wm, fb, Yi, d, BN, TY, je, ff, eH, eZ, aF, VI, iV, iR, bz, LB, es, bF, apM, Ew, zr, bE, AR, Hs, cP, adg, Pk, X, y); #c1(CD44) c2(NP_001189484) c3(CD44 antigen isoform 6 precursor) c4(80235, 80236, 80237) c5(80238, 80239, 80240, 80241, 80242, 80243) c6( ) c7(bM, T, aC, UH, Tb, fR, Zz, ea, if, e, fd, bB, qj, ci, lR, ip, x, afr, aE, il, rJ, pX, aeO, ajv, K, DY, H, pk, eL, aiJ, cp, aa, eG, yo, aeR, dH, JI, aW, Ls, adw, XV, fc, nY, g, as, pl, hJ, eF, cm, aHd, aAW, ale, lt, gt, nB, avZ, AV, eM, iA, iT, cj, k, m, iu, dD, vl, ajm, aEj, wu, sw, zX, QO, NT, fg, n, aj, kv, fr, qN, zP, cd, SW, ai, Bl, ft, jz, gd, kH, h, lG, By, hn, dL, ey, SH, hB, Ha, pl, yi, aiB, PX, aGZ, AQ, ahJ, aq, fe, Pp, kp, Hu, Ca, CM, Qy, bP, dB, hk, anZ, vb, hL, bm, E, fL, bD, it, B, bL, fM, dA, Dh, fG, yk, jx, aA, hy, v, j, aHa, bX, acn, aHc, V, pd, q, J, er, eA, eJ, Zp, aEE, mP, Vr, qK, HC, aHb, aT, j, Fg, ae, ne, af, bA, kl, s, vT, nO, oV, Y, ev, fO, iU, iZ, r, en, cl, t, ik, pb, ck, sS, nM, kP, Dy, ac, iM, nG, aGY, gX, wR, fD, kZ, bt, rA, gx, Gl, nZ, id, Mb, qz, iW, jN, jh, Fb, Gn, eq, EA, Fc, mC, ad, afb, aoH, abZ, dP, hX, hd, ib, av, Tg, db, NS, akJ, LA, io, hP, lQ, wm, fb, Yi, d, BN, TY, je, ff, eH, eZ, aF, VI, iV, iR, bz, LB, es, bF, apM, Ew, zr, bE, AR, Hs, cP, adg, Pk, X, y); #c1(CD44) c2(NP_001189485) c3(CD44 antigen isoform 7 precursor) c4(80244) c5(80245, 80246, 80247, 80248, 80249, 80250) c6( ) c7(bM, T, aC, UH, Tb, fR, Zz, ea, if, e, fd, bB, qj, ci, lR, ip, x, afr, aE, il, rJ, pX, aeO, ajv, K, DY, H, pk, eL, aiJ, cp, aa, eG, yo, aeR, dH, JI, aW, Ls, adw, XV, fc, nY, g, as, pL, hJ, eF, cm, aHd, aAW, ale, lt, gt, nB, avZ, AV, eM, iA, iT, cj, k, m, iu, dD, vl, ajm, aEj, wu, sw, zX, QO, NT, fg, n, aj, kv, fr, qN, zP, cd, SW, ai, Bl, ft, jz, gd, kH, h, lG, By, hn, dL, ey, SH, hB, Ha, pl, yi, aiB, PX, aGZ, AQ, ahJ, aq, fe, Pp, kp, Hu, Ca, cM, Qy, bP, dB, hk, anZ, vb, hL, bm, E, fL, bD, it, B, bL, fM, dA, Dh, fG, yk, jx, aA, hy, v, j, aHa, bX, acn, aHc, V, pd, q, J, er, eA, eJ, Zp, aEE, mP, Vr, qK, HC, aHb, aT, j, Fg, ae, ne, af, bA, kl, s, vT, nO, oV, Y, ev, fO, iU, iZ, r, en, cl, t, iK, pb, ck, sS, nM, kP, Dy, ac, iM, nG, aGY, gX, wR, fD, kZ, bt, rA, gx, Gl, nZ, id, Mb, qz, iW, jN, jh, Fb, Gn, eq, EA, Fc, mC, ad, afb, aoH, abZ, dP, hX, hd, ib, av, Tg, db, NS, akJ, LA, io, hP, lQ, wm, fb, Yi, d, BN, TY, je, ff, eH, eZ, aF, VI, iV, iR, bz, LB, es, bF, apM, Ew, zr, bE, AR, Hs, cP, adg, Pk, X, y); #c1(CD44) c2(NP_001189486) c3(CD44 antigen isoform & precursor) c4( ) c5(80251, 80252, 80253, 80254, 80255, 80256) c6( ) c7(bM, T, aC, UH, Tb, fR, Zz, ea, if, e, fd, bB, qj, ci, lR, ip, x, afr, aE, il, rJ, pX, aeO, ajv, K, DY, H, pk, eL, aiJ, cp, aa, eG, yo, aeR, dH, JI, aW, Ls, adw, XV, fc, nY, g, as, pl, hJ, eF, cm, aHd, aAW, ale, lt, gt, nB, avZ, AV, eM, iA, iT, cj, k, m, iu, dD, vl, ajm, aEj, wu, sw, zX, QO, NT, fg, n, aj, kv, fr, qN, zP, cd, SW, ai, Bl, ft, jz, gd, kH, h, lG, By, hn, dL, ey, SH, hB, Ha, pl, yi, aiB, PX, aGZ, AQ, ahJ, aq, fe, Pp, kp, Hu, Ca, cM, Qy, bP, dB, hk, anZ, vb, hL, bm, E, fL, bD, it, B, bL, fM, dA, Dh, fG, yk, jx, aA, hy, v, j, aHa, bX, acn, aHc, V, pd, q, J, er, eA, eJ, Zp, aEE, mP, Vr, qK, HC, aHb, aT, j, Fg, ae, ne, af, bA, kl, s, vT, nO, oV, Y, ev, fO, iU, iZ, r, en, cl, t, ik, pb, ck, sS, nM, kP, Dy, ac, iM, nG, aGY, gX, wR, fD, kZ, bt, rA, gx, Gl, nZ, id, Mb, qz, iW, jN, jh, Fb, Gn, eq, EA, Fc, mC, ad, afb, aoH, abZ, dP, hX, hd, ib, av, Tg, db, NS, akJ, LA, io, hP, lQ, wm, fb, Yi, d, BN, TY, je, ff, eH, eZ, aF, VI, iV, iR, bz, LB, es, bF, apM, Ew, zr, bE, AR, Hs, cP, adg, Pk, X, y); #c1(CD44) c2(XP_005253288) c3(CD44 antigen isoform X1) c4(272410) c5(272411, 272412, 272413, 272414, 272415, 272416) c6( ) c7(bM, T, aC, UH, Tb, fR, Zz, ea, if, e, fd, bB, qj, ci, lR, ip, x, afr, aE, il, rJ, pX, aeO, ajv, K, DY, H, pk, eL, aiJ, cp, aa, eG, yo, aeR, dH, JI, aW, Ls, adw, XV, fc, nY, g, as, pl, hJ, eF, cm, aHd, aAW, ale, lt, gt, nB, avZ, AV, eM, iA, iT, cj, k, m, iu, dD, vl, ajm, aEj, wu, sw, zX, QO, NT, fg, n, aj, kv, fr, qN, zP, cd, SW, ai, Bl, ft, jz, gd, kH, h, lG, By, hn, dL, ey, SH, hB, Ha, pl, yi, aiB, PX, aGZ, AQ, ahJ, aq, fe, Pp, kp, Hu, Ca, cM, Qy, bP, dB, hk, anZ, vb, hL, bm, E, fL, bD, it, B, bL, fM, dA, Dh, fG, yk, jx, aA, hy, v, j, aHa, bX, acn, aHc, V, pd, q, J, er, eA, eJ, Zp, aEE, mP, Vr, qK, HC, aHb, aT, j, Fg, ae, ne, af, bA, kl, s, vT, nO, oV, Y, ev, fO, iU, iZ, r, en, cl, t, ik, pb, ck, sS, nM, kP, Dy, ac, iM, nG, aGY, gX, wR, fD, kZ, bt, rA, gx, Gl, nZ, id, Mb, qz, iW, jN, jh, Fb, Gn, eq, EA, Fc, mC, ad, afb, aoH, abZ, dP, hX, hd, ib, av, Tg, db, NS, akJ, LA, io, hP, lQ, wm, fb, Yi, d, BN, TY, je, ff, eH, eL, aF, VI, iV, iR, bz, LB, es, bF, apM, Ew, zr, bE, AR, Hs, cP, adg, Pk, X, y); #c1(CD44) c2(XP_005253291) c3(CD44 antigen isoform X5) c4( ) c5(272417, 272418, 272419, 272420, 272421, 272422) c6( ) c7(bM, T, aC, UH, Tb, fR, Zz, ea, if, e, fd, bB, qj, ci, lR, ip, x, afr, aE, il, rJ, pX, aeI, ajv, K, DY, H, pk, eL, aiJ, cp, aa, eG, yo, aeR, dH, JI, aW, Ls, adw, XV, fc, nY, g, as, pl, hJ, eF, cm, aHd, aAW, aHe, lt, gt, nB, avZ, AV, eM, iA, lT, cj, k, m, iu, dD, vl, ajm, aEj, wu, sw, zX, QO, NT, fg, n, aj, kv, fr, qN, zP, cd, SW, ai, Bl, ft, jz, gd, kH, h, lG, By, hn, dL, ey, SH, hB, Ha, pl, yi, aiB, PX, aGZ, AQ, ahJ, aq, fe, Pp, kp, Hu, Ca, cM, Qy, bP, dB, hk, anZ, vb, hL, bm, E, fL, bD, it, B, bL, fM, dA, Dh, fG, yk, jx, aA, hy, v, j, aHa, bX, acn, aHc, V, pd, q, J, er, eA, eJ, Zp, aEE, mP, Vr, qK, HC, aHb, aT, j, Fg, ae, ne, af, bA, kl, s, vT, nO, oV, Y, ev, fO, iU, iZ, r, en, cl, t, ik, pb, ck, sS, nM, kP, Dy, ac, iM, nG, aGY, gX, wR, fD, kZ, bt, rA, gx, GI, nZ, id, Mb, qz, iW, jN, jh, Fb, Gn, eq, EA, Fc, mC, ad, afb, aoH, abZ, dP, hX, hd, ib, av, Tg, db, NS, akJ, LA, io, hP, lQ, wm, fb, Yi, d, BN, TY, je, ff, eH, eZ, aF, VI, lV, iR, bz, LB, es, bF, apM, Ew, zr, bE, AR, Hs, cP, adg, Pk, X, y); #c1 (CD44) c2(XP_006718451) c3(CD44 antigen isoform X3) c4(291875) c5( ) c6( ) c7(bM, T, aC, UH, Tb, fR, Zz, ea, if, e, fd, bB, qj, ci, lR, ip, x, afr, aE, il, rJ, pX, aeO, ajv, K, DY, H, pk, eL, aiJ, cp, aa, eG, yo, aeR, dH, JI, aW, Ls, adw, XV, fc, nY, g, as, pl, hJ, eF, cm, aHd, aAW, aHe, lt, gt, nB, avZ, AV, eM, iA, iT, cj, k, m, iu, dD, vl, ajm, aEj, wu, sw, zX, oO, NT, fg, n, aj, kv, fr, qN, zP, cd, SW, ai, Bl, ft, jz, gd, kH, h, lG, By, hn, dL, ey, SH, hB, Ha, pl, yi, aiB, PX, aGZ, AQ, ahJ, aq, fe, Pp, kp, Hu, Ca, cM, Qy, bP, dB, hk, anZ, vb, hL, bm, E, fL, bD, it, B, bL, fM, dA, Dh, fG, yk, jx, aA, hy, v, j, aHa, bX, acn, aHc, V, pd, q, J, er, eA, eJ, Zp, aEE, mP, Vr, qK, HC, aHb, aT, j, Fg, ae, ne, af, bA, kl, s, vT, nO, oV, Y, ev, fO, iU, iZ, r, en, cl, t, iK, pb, ck, sS, nM, kP, Dy, ac, iM, nG, aGY, gX, wR, fD, kZ, bt, rA, gx, GI, nZ, id, Mb, qz, iW, jN, jh, Fb, Gn, eq, EA, Fc, mC, ad, afb, aoH, abZ, dP, hX, hd, ib, av, Tg, db, NS, akJ, LA, io, hP, lQ, wm, fb, Yi, d, BN, TY, je, ff, eH, eZ, aF, VI, iV, iR, bz, LB, es, bF, apM, Ew, zr, bE, AR, Hs, cP, adg, Pk, X, y); #c1(CD44) c2(XP_011518784) c3(CD44 antigen isoform X7) c4(313181) c5(313182, 313183, 313184, 313185, 313186, 313187) c6( ) c7(bM, T, aC, UH, Tb, fR, Zz, ea, if, e, fd, bB, qj, ci, lR, ip, x, afr, aE, il, rJ, pX, aeO, ajv, K, DY, H, pk, eL, aiJ, cp, aa, eG, yo, aeR, dH, JI, aW, Ls, adw, XV, fc, nY, g, as, pl, hJ, eF, cm, aHd, aAW, ale, lt, gt, nB, avZ, AV, eM, iA, iT, cj, k, m, iu, dD, vl, ajm, aEj, wu, sw, zX, oO, NT, fg, n, aj, kv, fr, qN, zP, cd, SW, ai, Bl, ft, jz, gd, kH, h, lG, By, hn, dL, ey, SH, hB, Ha, pl, yi, aiB, PX, aGZ, AQ, ahJ, aq, fe, Pp, kp, Hu, Ca, cM, Ly, bP, dB, hk, anZ, vb, hL, bm, E, fL, bD, it, B, bL, fM, dA, Dh, fG, yk, jx, aA, hy, v, j, aHa, bX, acn, aHc, V, pd, q, J, er, eA, eJ, Zp, aEE, mP, Vr, qK, HC, aHb, aT, j, Fg, ae, ne, af, bA, kl, s, vT, nO, oV, Y, ev, fO, iU, iZ, r, en, cl, t, ik, pb, ck, sS, nM, kP, Dy, ac, iM, nG, aGY, gX, wR, fD, kZ, bt, rA, gx, Gl, nZ, id, Mb, qz, iW, jN, jh, Fb, Gn, eq, EA, Fc, mC, ad, afb, aoH, abZ, dP, hX, hd, ib, av, Tg, db, NS, akJ, LA, io, hP, lQ, wm, fb, Yi, d, BN, TY, je, ff, eH, eZ, aF, VI, iV, iR, bz, LB, es, bF, apM, Ew, zr, bE, AR, Hs, cP, adg, Pk, X, y); #c1(CD44) c2(XP_011518790) c3(CD44 antigen isoform X16) c4(313188) c5(313189, 313190, 313191, 313192, 313193, 313194) c6( ) c7(bM, T, aC, UH, Tb, fR, Zz, ea, if, e, fd, bB, qj, ci, lR, ip, x, afr, aE, il, rJ, pX, aeO, ajv, K, DY, H, pk, eL, aiJ, cp, aa, eG, yo, aeR, dH, JI, aW, Ls, adw, XV, fc, nY, g, as, pl, hJ, eF, cm, aHd, aAW, aHe, lt, gt, nB, avZ, AV, eM, iA, iT, cj, k, m, iu, dD, vl, ajm, aEj, wu, sw, zX, QO, NT, fg, n, aj, kv, fr, qN, zP, cd, SW, ai, Bl, ft, jz, gd, kH, h, lG, By, hn, dL, ey, SH, hB, Ha, pl, yi, aiB, PX, aGZ, AQ, ahJ, aq, fe, Pp, kp, Hu, Ca, CM, Qy, bP, dB, hk, anZ, vb, hL, bm, E, fL, bD, it, B, bL, fM, dA, Dh, fG, yk, jx, aA, hy, v, j, aHa, bX, acn, aHc, V, pd, q, J, er, eA, eJ, Zp, aEE, mP, Vr, qK, HC, aHb, aT, j, Fg, ae, ne, af, bA, kl, s, vT, nO, oV, Y, ev, fO, iU, iZ, r, en, cl, t, iK, pb, ck, sS, nM, kP, Dy, ac, iM, nG, aGY, gX, wR, fD, kZ, bt, rA, gx, Gl, nZ, id, Mb, qz, iW, jN, jh, Fb, Gn, eq, EA, Fc, mC, ad, afb, aoH, abZ, dP, hX, hd, ib, av, Tg, db, NS, akJ, LA, io, hP, lQ, wm, fb, Yi, d, BN, TY, je, ff, eH, eZ, aF, VI, iV, iR, bz, LB, es, bF, apM, Ew, zr, bE, AR, Hs, cP, adg, Pk, X, y); #c1(CD46) c2(NP_002380) c3(membrane cofactor protein isoform 1 precursor) c4(127987) c5(127988, 127989, 127990, 127991, 127992, (27993) c6( ) c7(T, FR, if, ci, aY, K, aHf, aHg, aW, uY, pD, g, as, eF, cm, wT, eM, acw, Hb, m, hh, pJ, n, uE, qN, cd, ai, ke, ql, h, db, Cw, EU, aq, CQ, iO, ok, W, dB, eD, at, eo, B, EM, eR, AL, KA, V, eA, zT, Yn, aT, GQ, cy, ae, af, Uf, Y, lo, en, t, ik, ali, sn, hc, DW, dy, JH, bH, hS, iW, oA, jh, hF, ad, ib, oj, aHh, aGF, qZ, ff, eH, eZ, bz, LB, lM, DH); #c1(CD46) c2(NP_722548) c3(membrane cofactor protein isoform 4 precursor) c4(246821, 246822) c5(246823, 246824, 246825, 246826, 246827, 246828) c6( ) c7(T, fR, if, ci, aY, K, aHf, aHg, aW, uY, pD, g, as, eF, cm, wT, eM, acw, Hb, m, hh, pJ, n, uE, qN, cd, ai, ke, ql, h, db, Cw, EU, aq, CO, iO, ok, W, dB, eD, at, eo, B, EM, eR, AL, KA, V, eA, zT, Yn, aT, GQ, cy, ae, af, Uf, Y, lo, en, t, ik, aHi, sn, hc, DW, dy, JH, bH, hS, iW, oA, jh, hF, ad, ib, oj, aHh, aGF, qZ, ff, eH, eZ, bz, LB, lM, DH); #c1(CD46) c2(NP_758860) c3(membrane cofactor protein isoform 14 precursor) c4(249294) c5(249295, 249296, 249297, 249298, 249299, 249300, 249301, 249302, 249303, 249304, 249305, 249306) c6( ) c7(T, FR, if, ci, aY, K, aHf, aHg, aW, uY, pD, g, as, eF, cm, wT, eM, acw, Hb, m, hh, pJ, n, uE, qN, cd, ai, ke, ql, h, db, Cw, EU, aq, CO, iO, ok, W, dB, eD, at, eo, B, EM, eR, AL, KA, V, eA, zT, Yn, aT, GQ, cy, ae, af, Uf, Y, lo, en, t, ik, aHi, sn, hc, DW, dy, JH, bH, hS, iW, oA, jh, hF, ad, ib, oj, aHh, aGF, qZ, ff, eH, eZ, bz, LB, lM, DH); #c1(CD46) c2(NP_758861) c3(membrane cofactor protein isoform 3 precursor) c4(249307) c5(249308, 249309, 249310, 249311, 249312, 249313) c6( ) c7(T, FR, if, ci, aY, K, aHf, aHg, aW, uY, pD, g, as, eF, cm, wT, eM, acw, Hb, m, hh, pJ, n, uE, qN, cd, ai, ke, ql, h, db, Cw, EU, aq, CQ, iO, ok, W, dB, eD, at, eo, B, EM, eR, AL, KA, V, eA, zT, Yn, aT, GQ, cy, ae, af, Uf, Y, lo, en, t, ik, aHi, sn, hc, DW, dy, JH, bH, hS, iW, oA, jh, hF, ad, ib, oj, aHh, aGF, qZ, ff, eH, eZ, bz, LB, lM, DH); #c1(CD46) c2(NP_758862) c3(membrane cofactor protein isoform 5 precursor) c4(249314) c5(249315, 249316, 249317, 249318, 249319, 249320) c6( ) c7(T, fR, if, ci, aY, K, aHf, aHg, aW, uY, pD, g, as, eF, cm, wT, eM, acw, Hb, m, hh, pJ, n, uE, qN, cd, ai, ke, ql, h, db, Cw, EU, aq, CO, iO, ok, W, dB, eD, at, eo, B, EM, eR, AL, KA, V, eA, zT, Yn, aT, GQ, cy, ae, af, Uf, Y, lo, en, t, ik, aHi, sn, hc, DW, dy, JH, bH, hS, iW, oA, jh, hF, ad, ib, oj, aHh, aGF, qZ, ff, eH, eZ, bz, LB, lM, DH); #c1(CD46) c2(NP_758863) c3(membrane cofactor protein isoform 6 precursor) c4(249321) c5(249322, 249323, 249324, 249325, 249326, 249327) c6( ) c7(T, FR, if, ci, aY, K, aHf, aHg, aW, uY, pD, g, as, eF, cm, wT, eM, acw, Hb, m, hh, pJ, n, uE, qN, cd, ai, ke, ql, h, db, Cw, EU, aq, CO, iO, ok, W, dB, eD, at, eo, B, EM, eR, AL, KA, V, eA, zT, Yn, aT, GQ, cy, ae, af, Uf, Y, lo, en, t, ik, ali, sn, hc, DW, dy, JH, bH, hS, iW, oA, jh, hF, ad, ib, oj, aHh, aGF, qZ, ff, eH, eZ, bz, LB, lM, DH); #c1(CD46) c2(NP_758869) c3(membrane cofactor protein isoform 2 precursor) c4(249328, 249329) c5(249330, 249331, 249332, 249333, 249334, 249335, 249336, 249337, 249338, 249339, 249340, 249341) c6( ) c7(T, fR, if, ci, aY, K, aHf, aHg, aW, uY, pD, g, as, eF, cm, wT, eM, acw, Hb, m, hh, pJ, n, uE, qN, cd, ai, ke, ql, h, db, Cw, EU, aq, CO, iO, ok, W, dB, eD, at, eo, B, EM, eR, AL, KA, V, eA, zT, Yn, aT, GQ, cy, ae, af, Uf, Y, lo, en, t, ik, ali, sn, hc, DW, dy, JH, bH, hS, iW, oA, jh, hF, ad, ib, oj, aHh, aGF, qZ, ff, eH, eZ, bz, LB, lM, DH); #c1(CD46) c2(NP_758871) c3(membrane cofactor protein isoform 12 precursor) c4(249342) c5(249343, 249344, 249345, 249346, 249347, 249348) c6( ) c7(T, FR, if, ci, aY, K, aHf, aHg, aW, uY, pD, g, as, eF, cm, wT, eM, acw, Hb, m, hh, pJ, n, uE, qN, cd, ai, ke, ql, h, db, Cw, EU, aq, CO, iO, ok, W, dB, eD, at, eo, B, EM, eR, AL, KA, V, eA, zT, Yn, aT, GQ, cy, ae, af, Uf, Y, lo, en, t, ik, aHi, sn, hc, DW, dy, JH, bH, hS, iW, oA, jh, hF, ad, ib, oj, aHh, aGF, qZ, ff, eH, eZ, bz, LB, lM, DH); #c1(CD46) c2(XP_011507865) c3(membrane cofactor protein isoform X1) c4(298029) c5(298030, 298031, 298032, 298033, 298034, 298035) c6( ) c7(T, fR, if, ci, aY, K, aHf, aHg, aW, uY, pD, g, as, eF, cm, wT, eM, acw, Hb, m, hh, pJ, n, uE, qN, cd, ai, ke, ql, h, db, Cw, EU, aq, CO, iO, ok, W, dB, eD, at, eo, B, EM, eR, AL, KA, V, eA, zT, Yn, aT, GQ, cy, ae, af, Uf, Y, lo, en, t, ik, aHi, sn, hc, DW, dy, JH, bH, hS, iW, oA, jh, hF, ad, ib, oj, aHh, aGF, qZ, ff, eH, eZ, bz, LB, lM, DH); #c1(CD46) c2(XP_011507866) c3(membrane cofactor protein isoform X2) c4(298036) c5(298037, 298038, 298039, 298040, 298041, 298042) c6( ) c7(T, FR, if, ci, aY, K, alf, alg, aW, uY, pD, g, as, eF, cm, wT, eM, acw, Hb, m, hh, pJ, n, uE, qN, cd, ai, ke, ql, h, db, Cw, EU, aq, CQ, iO, ok, W, dB, eD, at, eo, B, EM, eR, AL, KA, V, eA, zT, Yn, aT, GQ, cy, ae, af, Uf, Y, lo, en, t, ik, ali, sn, hc, DW, dy, JH, bH, hS, iW, DA, jh, hF, ad, ib, oj, aHh, aGF, qZ, ff, eH, eZ, bz, LB, lM, DH); #c1(CD47) c2(NP_001768) c3(leukocyte surface antigen CD47 isoform 1 precursor) c4(120964) c5(120965, 120966, 120967, 120968, 120969, 120970) c6( ) c7(ID, T, if, eX, jh, fd, n, ci, qN, PJ, pz, V, aHj, ai, ad, aE, Dq, dv, aT, kf, cy, vH, dL, eV, K, jj, DY, ae, eL, cp, ly, d, MD, nO, aW, Y, oV, hl, aq, S, as, pm, aaO, iR, ik, bz, bw, lx, yR, LB, ck, VJ, kP, ac, zr, bE, lT, HN, cj, eo, k, hh, bt, kS, B); #c1(CD47) c2(NP_942088) c3(leukocyte surface antigen CD47 isoform 2 precursor) c4(260452) c5(260453, 260454, 260455, 260456, 260457, 260458) c6( ) c7(ID, T, if, eX, jh, fd, n, ci, qN, PJ, pz, V, aHj, ai, ad, aE, Dq, dv, aT, kf, cy, vH, dL, eV, K, jj, DY, ae, eL, cp, ly, d, MD, nO, aW, Y, oV, hl, aq, S, as, pm, aaO, iR, ik, bz, bw, lx, yR, LB, ck, VJ, kP, ac, zr, bE, iT, HN, cj, eo, k, hh, bt, kS, B); #c1(CD48) c2(NP_001769) c3(CD48 antigen isoform 1 precursor) c4(120971) c5(120972, 120973, 120974, 120975, 120976, 120977) c6( ) c7(T, jh, mC, ad, x, aT, ae, HZ, aW, Pg, eZ, bw, mk, ck, kz, wR, dW, fL, k, y, bt, fh); #c1(CD48) c2(XP_005245682) c3(CD48 antigen isoform X2) c4(266901) c5(266902, 266903, 266904, 266905, 266906, 266907) c6( ) c7(T, jh, mC, ad, x, aT, ae, HZ, aW, Pg, eZ, bw, mK, ck, kz, wR, dW, fl, k, y, bt, fh); #c1(CD5) c2(NP_055022) c3(T-cell surface glycoprotein CDS precursor) c4(178334) c5(178335, 178336, 178337, (78338, 178339, 178340) c6( ) c7(Cv, ck, T, fM, iM, je, iW, jh, cj, fd, hn, aHk, y); #c1(CD55) c2(NP_000565) c3(complement decay-accelerating factor isoform 1 preproprotein) c4(12713, 12714) c5(12715, 12716, 12717, 12718, 12719, 12720, 12721, 12722, 12723, 12724, 12725, 12726, 12727, 12728, 12729, 12730, 12731, 12732, 12733, 12734, 12735, 12736, 12737) c6( ) c7(oO, sY, T, FR, iW, jN, jh, bB, mN, j, ci, hF, aHj, q, br, ad, ai, dP, aT, lG, ji, K, wt, xa, aHo, aHp, hM, iU, aW, r, aq, lo, aF, as, iV, t, Bu, uC, f, cm, bw, LB, yd, ck, aHm, W, aHn, bE, kz, X, cj, uJ, dW, m, y, fh, B, aHI); #c1 (CD55) c2(NP_001108224) c3(complement decay-accelerating factor isoform 2 precursor) c4(43161, 43162) c5(43163, 43164, 43165, 43166, 43167, 43168) c6( ) c7(oO, sY, T, FR, iW, jN, jh, bB, mN, j, ci, hF, aHj, q, br, ad, ai, dP, aT, lG, ji, K, wt, xa, aHo, aHp, hM, iU, aW, r, aq, lo, aF, as, iV, t, Bu, uC, f, cm, bw, LB, yd, ck, aHm, W, aHn, bE, kz, X, cj, uJ, dW, m, y, fh, B, aHI); #c1(CD55) c2(NP_001287831) c3(complement decay-accelerating factor isoform 5 precursor) c4( ) c5(112967) c6( ) c7(oO, sY, T, FR, iW, jN, jh, bB, mN, j, ci, hF, aHj, q, br, ad, ai, dP, aT, lG, ji, K, wt, xa, aHo, aHp, hM, iU, aW, r, aq, lo, aF, as, iV, t, Bu, uC, f, cm, bw, LB, yd, ck, aHm, W, aHn, bE, kz, X, cj, uJ, dW, m, y, fh, B, aHI); #c1(CD55) c2(NP_001287832) c3(complement decay-accelerating factor isoform 6 precursor) c4(112968) c5(112969, 112970, 112971, 112972, 112973, 112974, 112975, 112976, 112977) c6( ) c7(oO, sY, T, FR, iW, jN, jh, bB, mN, j, ci, hF, aHj, q, br, ad, ai, dP, aT, lG, ji, K, wt, xa, aHo, aHp, hM, iU, aW, r, aq, lo, aF, as, iV, t, Bu, uC, f, cm, bw, LB, yd, ck, aHm, W, aHn, bE, kz, X, cj, uJ, dW, m, y, fh, B, aHI); #c1(CD55) c2(NP_001287833) c3(complement decay-accelerating factor isoform 7 precursor) c4(112978, 112979) c5(112980, 112981, 112982, 112983, 112984, 112985) c6( ) c7(oO, sY, T, FR, iW, jN, jh, bB, mN, j, ci, hF, aHj, q, br, ad, ai, dP, aT, lG, ji, K, wt, xa, aHo, aHp, hM, iU, aW, r, aq, lo, af, as, iV, t, Bu, uC, f, cm, bw, LB, yd, ck, aHm, W, aHn, bE, kz, X, cj, ud, dW, m, y, fh, B, aHI); #c1(CD59) c2(NP_001120695) c3(CD59 glycoprotein preproprotein) c4(44631) c5(44632, 44633, 44634, 44635, 44636, 44637, 44638, 44639, 44640, 44641, 44642, 44643, 44644, 44645, 44646, 44647, 44648, 44649, 44650, 44651, 44652, 44653, 44654, 44655, 44656, 44657, 44658, 44659, 44660, 44661, 44662, 44663, 44664, 44665, 44666, 44667, 44668, 44669, 44670, 44671, 44672, 44673, 44674, 44675, 44676, 44677, 44678, 44679, 44680, 44681, 44682, 44683, 44684, 44685, 44686, 44687) c6( ) c7(aHq, T, fR, ea, e, ci, aY, gH, K, aHo, azh, aa, xQ, EV, aW, zC, g, cm, bw, GR, Uk, yd, eM, TG, mx, vy, cj, dW, m, dD, aw, cQ, aj, ct, nA, aHj, cd, br, ai, eW, b, cc, lG, jy, ey, alY, A, MD, EU, aq, mo, W, Fi, kz, ajn, mg, bm, nl, va, B, tq, bL, aHI, eR, aA, cz, v, j, nS, q, J, er, aT, yu, Oi, ae, af, iU, Y, fO, r, en, t, f, ck, cl, hc, fP, mf, dy, bt, ao, qb, aZ, iW, jh, Hn, bS, dG, jE, eq, hF, ad, hX, fU, oW, vc, aHp, aGF, Gy, lR, cW, ff, aF, hp, avG, Z, HI, bz, axs, ei, i, y, fh, X); #c1 (CD5L) c2(NP_005885) c3(CD5 antigen-like precursor) c4(160112) c5(160113, 160114, 160115, 160116, 160117, 160118) c6( ) c7(aE, pZ, WE, EQ, hD, fe, dw, Fg, af); #c1(CD6) c2(NP_001241679) c3(T-cell differentiation antigen CD6 isoform 2 precursor) c4(89041) c5(89042, 89043, 89044, 89045, 89046, 89047) c6( ) c7(aiu, aiF, T, fM, YU, FG, aHA, aHt, aHx, jh, aHv, oY, aHC, jE, ci, aHy, aY, V, hF, avD, br, ip, afb, lS, iJ, x, b, fX, aHu, Rq, pe, i, Bj, zK, aT, aHs, dz, hn, rO, K, in, jj, pk, dx, cp, Nr, iU, et, pr, aW, Y, TH, FD, bw, po, aHw, hf, eF, f, adj, aHr, QA, atb, op, iN, Nq, ck, ou, Dy, iT, vA, au, aHB, cj, hj, pg, Fk, k, bC, azr, aiG, aHz, bt, y); #c1(CD6) c2(NP_001241680) c3(T-cell differentiation antigen CD6 isoform 3 precursor) c4(89048) c5(89049, 89050, 89051, 89052, 89053, 89054) c6( ) c7(aiu, aiF, T, fM, YU, FG, aHA, aHt, aHx, jh, aHv, oY, aHC, jE, ci, aHy, aY, V, hF, avD, br, ip, afb, lS, iJ, x, b, fX, aHu, Rq, pe, i, Bj, zK, aT, als, dz, hn, rO, K, in, jj, pk, dx, cp, Nr, iU, et, pr, aW, Y, TH, FD, bw, po, aHw, hf, eF, f, adj, aHr, QA, atb, op, iN, Nq, ck, ou, Dy, iT, vA, au, aHB, cj, hj, pg, Fk, k, bC, azr, aiG, aHz, bt, y); #c1(CD6) c2(NP_006716) c3(T-cell differentiation antigen CDG isoform 1 precursor) c4(167378) c5(167379, 167380, 167381, 167382, 167383, 167384) c6( ) c7(aiu, aiF, T, fM, YU, FG, aHA, aHt, aHx, jh, aHv, oY, aHC, jE, ci, aHy, aY, V, hF, avD, br, ip, afb, lS, iJ, x, b, fX, aHu, Rq, pe, i, Bj, zK, aT, aHs, dz, hn, rO, K, in, jj, pk, dx, cp, Nr, iU, et, pr, aW, Y, TH, FD, bw, po, aHw, hf, eF, f, adj, aHr, QA, atb, op, iN, Nq, ck, ou, Dy, iT, vA, au, aHB, cj, hj, pg, Fk, k, bC, azr, aiG, aHz, bt, y); #c1(CD6) c2(XP_006718801) c3(T-cell differentiation antigen CD6 isoform X2) c4(292004) c5(292005, 292006, 292007, 292008, 292009, 292010) c6( ) c7(aiu, aiF, T, fM, YU, FG, aHA, aHt, aHx, jh, aHv, oY, aHC, jE, ci, aHy, aY, V, hF, avD, br, ip, afb, lS, iJ, x, b, fx, aHu, Rq, pe, i, Bj, zK, aT, als, dz, hn, rO, K, in, jj, pk, dx, cp, Nr, iU, et, pr, aW, Y, TH, FD, bw, po, aHw, hf, eF, f, adj, aHr, QA, atb, op, iN, Nq, ck, ou, Dy, iT, vA, au, aHB, cj, hj, pg, Fk, k, bC, azr, aiG, aHz, bt, y); #c1(CD6) c2(XP_006718804) c3(T-cell differentiation antigen CD6 isoform X6) c4(292011) c5(292012, 292013, 292014, 292015, 292016, 292017) c6( ) c7(aiu, aiF, T, fM, YU, FG, aHA, aHt, aHx, jh, aHv, oY, aHC, jE, ci, aHy, aY, V, hF, avD, br, ip, afb, lS, iJ, x, b, fx, aHu, Rq, pe, i, Bj, zK, aT, aHs, dz, hn, rO, K, in, jj, pk, dx, cp, Nr, iU, et, pr, aW, Y, TH, FD, bw, po, aHw, hf, eF, f, adj, aHr, DA, atb, op, iN, Nq, ck, ou, Dy, iT, vA, au, aHB, cj, hj, pg, Fk, k, bC, azr, aiG, aHz, bt, y); #c1(CD63) c2(NP_001244321) c3(CD63 antigen isoform C) c4(92156) c5(92157, 92158, 92159, 92160, 92161, 92162) c6( ) c7(T, fR, jp, bB, Fc, eA, aT, h, By, aHD, Cy, cp, aHE, tN, d, aW, Y, oV, g, as, pm, t, Vk, Hp, cT, nM, Dy, pc, aki, bE, at, pg, m, y); #c1 (CD63) c2(NP_001244330) c3(CD63 antigen isoform D precursor) c4( ) c5(92181, 92182, 92183, 92184, 92185, 92186, 92187, 92188) c6( ) c7(T, fR, jp, bB, Fc, eA, aT, h, By, aHD, Cy, cp, aHE, tN, d, aW, Y, oV, g, as, pm, t, Vk, Hp, cT, nM, Dy, pc, aki, bE, at, pg, m, y); #c1 (CD63) c2(NP_001254627) c3(CD63 antigen isoform A) c4(94674, 94675, 94676, 94677, 94678, 94679) c5(94680, 94681, 94682, 94683, 94684, 94685, 94686, 94687, 94688, 94689, 94690, 94691, 94692, 94693, 94694, 94695, 94696, 94697, 94698, 94699, 94700, 94701, 94702, 94703, 94704, 94705, 94706, 94707, 94708, 94709, 94710, 94711, 94712, 94713, 94714, 94715, 94716, 94717, 94718, 94719) c6( ) c7(T, fR, jp, bB, Fc, eA, aT, h, By, aHD, Cy, cp, aHE, tN, d, aW, Y, oV, g, as, pm, t, Vk, Hp, cT, nM, Dy, pc, aki, bE, at, pg, m, y); #c1(CD68) c2(NP_001035148) c3(macrosialin isoform B precursor) c4(32531) c5(32532, 32533, 32534, 32535, 32536, 32537) c6( ) c7(bM, T, apr, ea, l, fd, bB, ci, aY, aR, ip, aao, xE, rw, K, de, H, li, aW, lh, pl, bw, GR, iP, y, Qw, m, cZ, dD, zt, fr, PJ, TI, cd, b, h, By, ey, dd, Xh, nP, mR, anP, fe, aFx, JM, bm, at, L, bL, eR, jx, aA, v, j, mP, hW, aT, cy, bq, KU, af, bf, iU, oV, Y, r, lo, t, pb, aHG, dS, sS, iM, mf, fD, zu, vi, kh, atY, aZ, eq, atK, iJ, ad, dP, aHF, av, aaZ, F, ib, ach, ul, hZ, je, cW, cr, yy, aF, Fq, iV, bz, aS, i, hE, jr); #c1(CD68) c2(NP_001242) c3(macrosialin isoform A precursor) c4(89513) c5( ) c6( ) c7(bM, T, apr, ea, l, fd, bB, ci, aY, aR, ip, aao, xE, rw, K, de, H, li, aW, lh, pl, bw, GR, iP, y, Qw, m, cZ, dD, zt, fr, PJ, TI, cd, b, h, By, ey, dd, Xh, nP, mR, anP, fe, aFx, JM, bm, at, L, bL, eR, jx, aA, v, j, mP, hW, aT, cy, bq, KU, af, bf, iU, oV, Y, r, lo, t, pb, aHG, dS, sS, iM, mf, fD, zu, vi, kh, atY, aZ, eq, atK, iJ, ad, dP, aHF, av, aaZ, F, ib, ach, ul, hZ, je, cW, cr, yy, aF, Fq, iV, bz, aS, i, hE, jr); #c1(CD69) c2(NP_001772) c3(early activation antigen CD69) c4(120978, 120979) c5(120980, 120981, 120982, 120983, 120984, 120985, 120986, 120987, 120988, 120989, 120990, 120991) c6( ) c7(gx, bM, dA, aHH, jh, wV, lR, jE, wy, ai, cd, eA, ip, x, cc, F, xE, g, aE, h, hK, hH, Fg, ae, hM, A, mR, bf, oV, WE, yL, fO, CM, eZ, fe, BG, dw, bw, ik, pb, Wt, pZ, cj, k, be, y, B); #c1(CD7) c2(NP_006128) c3(T-cell antigen CD7 precursor) c4(162274) c5(162275, 162276, 162277, 162278, 162279, 162280) c6( ) c7(x, xR, auT, k, PJ, hF, bt, hS); #c1(CD72) c2(NP_001773) c3(B-cell differentiation antigen CD72) c4(120992, 120993, 120994, 120995) c5(120996, 120997, 120998, 120999, 121000, 121001, 121002, 121003, 121004, 121005, 121006, 121007, 121008, 121009, 121010, 121011) c6( ) c7(x, fl, DY, k, eZ); #c1(CD72) c2(XP_011516377) c3(B-cell differentiation antigen CD72 isoform X3) c4( ) c5(309598, 309599, 309600, 309601, 309602, 309603, 309604, 309605, 309606, 309607, 309608, 309609, 309610) c6( ) c7(x, fL, DY, k, eZ); #c1(CD74) c2(NP_001020329) c3(HLA class 11 histocompatibility antigen gamma chain isoform c) c4(28574) c5(28575, 28576, 28577, 28578, 28579, 28580, 28581, 28582, 28583, 28584, 28585, 28586, 28587, 28588, 28589, 28590, 28591) c6( ) c7(T, dD, ea, e, qy, fd, vr, jh, eq, ci, bh, fr, cd, mC, ad, eA, dP, av, ib, aE, py, aEu, aT, h, cy, bc, d, s, nO, Y, ff, lb, eZ, dN, dq, lo, hp, en, g, iV, cM, bz, es, eM, iA, cj, aEs, at, fL, B); #c1(CD74) c2(NP_001020330) c3(HLA class II histocompatibility antigen gamma chain isoform a) c4(28592, 28593) c5(28594, 28595, 28596, 28597, 28598, 28599, 28600, 28601, 28602, 28603, 28604, 28605, 28606, 28607, 28608, 28609) c6( ) c7(T, dD, ea, e, qy, fd, vr, jh, eq, ci, bh, fr, cd, mC, ad, eA, dP, av, ib, aE, py, aEu, aT, h, cy, bc, d, s, nO, Y, ff, lb, eZ, dN, dq, lo, hp, en, g, iV, cM, bz, es, eM, iA, cj, aEs, at, fL, B); #c1(CD74) c2(NP_004346) c3(HLA class II histocompatibility antigen gamma chain isoform b) c4(145538, 145539, 145540) c5(145541, 145542, 145543, 145544, 145545, 145546) c6( ) c7(T, dD, ea, e, qy, fd, vr, jh, eq, ci, bh, fr, cd, mC, ad, eA, dP, av, ib, aE, py, aEu, aT, h, cy, bc, d, s, no, Y, ff, lb, eZ, dN, dq, lo, hp, en, g, iV, cM, bz, es, eM, iA, cj, aEs, at, fL, B); #c1(CD79A) c2(NP_001774) c3(B-cell antigen receptor complex-associated protein alpha chain isoform 1 precursor) c4(121012) c5(121013, 121014, 121015, 121016, (21017, 121018) c6( ) c7(T, aov, fM, aBe, iW, jh, fd, aHI, hF, x, CJ, pe, pp, OG, ds, aT, h, ey, bc, Of, Og, bz, ck, amS, Zg, cj, k, bt, Ze); #c1(CD79A) c2(NP_067612) c3(B-cell antigen receptor complex-associated protein alpha chain isoform 2 precursor) c4(209052) c5(209053, 209054, 209055, 209056, 209057, 209058) c6( ) c7(T, aov, fM, aBe, iW, jh, fd, aHI, hF, x, CJ, pe, pp, OG, ds, aT, h, ey, bc, Of, Og, bz, ck, amS, Zg, cj, k, bt, Ze); #c1(CD79B) c2(NP_000617) c3(B-cell antigen receptor complex-associated protein beta chain isoform 1 precursor) c4(13963) c5(13964, 13965, 13966, 13967, 13968, 13969, 13970, 13971) c6( ) c7(gV, T, fM, iW, ex, jh, fd, apZ, aHK, zn, x, afm, aHJ, eV, hG, hl, eZ, hf, ck, ou, cj, Ze); #c1(CD79B) c2(NP_001035022) c3(B-cell antigen receptor complex-associated protein beta chain isoform 3 precursor) c4(32430) c5(32431, 32432, 32433, 32434, 32435, 32436) c6( ) c7(gV, T, fM, iW, eX, jh, fd, apZ, aHK, zn, x, afm, aHJ, eV, hG, hl, eZ, hf, ck, ou, cj, Ze); #c1(CD79B) c2(NP_067613) c3(B-cell antigen receptor complex-associated protein beta chain isoform 2 precursor) c4(209059) c5(209060, 209061, 209062, 209063, 209064, 209065) c6( ) c7(gV, T, fM, iW, eX, jh, fd, apZ, aHK, zn, x, afm, aHJ, eV, hG, hl, eZ, hf, ck, ou, cj, Ze); #c1(CD80) c2(NP_005182) c3(T-lymphocyte activation antigen CD80 precursor) c4(153684) c5(153685, 153686, 153687, 153688, 153689, 153690, 153691, 153692, 153693, 153694) c6( ) c7(bM, T, fR, if, fd, qj, ci, aR, x, xy, Ds, hu, eL, bc, aW, g, cm, iP, ba, eM, cj, k, m, ul, ht, aj, fr, qN, eW, aHN, h, eV, dL, ey, xP, Nr, A, po, dB, at, bk, FL, B, Dw, fM, jx, eX, aA, zN, j, C, aHM, V, q, J, eA, mP, aT, PK, iU, Y, bg, r, lo, en, ck, iM, yW, fP, bt, gx, id, axd, jh, ah, fu, mC, Pm, wD, hX, ds, lQ, ul, je, qZ, ff, eZ, aF, xH, hp, iV, bz, Nq, aHL, ME, y, iu); #c1(CD80) c2(XP_011511628) c3(T-lymphocyte activation antigen CD80 isoform X1) c4(302377, 302378) c5(302379, 302380, 302381, 302382, 302383, 302384, 302385, 302386, 302387, 302388, 302389, 302390) c6( ) c7(bM, T, fR, if, fd, qj, ci, aR, x, xy, Ds, hu, eL, bc, aW, g, cm, iP, ba, eM, cj, k, m, ul, ht, aj, fr, qN, eW, aHN, h, eV, dL, ey, xP, Nr, A, po, dB, at, bk, fL, B, Dw, fM, jx, eX, aA, zN, j, C, aHM, V, q, J, eA, mP, aT, PK, iU, Y, bg, r, lo, en, ck, iM, yW, fP, bt, gx, id, axd, jh, ah, fu, mC, Pm, wD, hX, ds, lQ, ul, je, qZ, ff, eZ, aF, xH, hp, iV, bz, Nq, aHL, ME, y, iu); #c1 (CD81) c2(NP_004347) c3(CD81 antigen isoform 1) c4(145547) c5(145548, 145549, 145550, 145551, 145552, 145553) c6( ) c7(fR, qb, yk, jx, ml, fd, v, aHO, eA, F, jZ, dV, h, dL, on, Y, aq, lo, cm, Wt, Gs, at, XO, B, vi); #c1(CD82) c2(NP_001020015) c3(CD82 antigen isoform 2) c4(28037, 28038, 28039) c5(28040, 28041, 28042, 28043, 28044, 28045, 28046, 28047, 28048, 28049, 28050, 28051, 28052, 28053, 28054) c6( ) c7(T, id, dA, Uc, rQ, if, WV, aA, cz, n, bB, v, fr, ci, qN, rP, fd, AO, q, ad, aHR, dP, aE, db, yt, aT, h, Du, dL, ae, eL, cp, pl, atX, RT, s, qt, aW, Y, ff, r, aq, Ej, af, en, cl, g, t, cm, bz, aHQ, iR, ck, es, bF, eM, nB, bE, gX, hL, m, aHP, y, it, B); #c1(CD82) c2(XP_006718285) c3(CD82 antigen isoform X2) c4(291669, 291670) c5(291671, 291672, 291673, 291674, 291675, 291676) c6( ) c7(T, id, dA, Uc, rd, if, WV, aA, cz, n, bB, v, fr, ci, qN, rP, fd, AO, q, ad, aHR, dP, aE, db, yt, aT, h, Ou, dL, ae, eL, cp, pl, atX, RT, s, qt, aW, Y, ff, r, aq, Ej, aF, en, cl, g, t, cm, bz, aHQ, iR, ck, es, bF, eM, nB, bE, gX, hL, m, aHP, y, it, B); #c1(CD82) c2(XP_006718286) c3(CD82 antigen isoform X1) c4(291677) c5(291678, 291679, 291680, 291681, 291682, 291683, 291684, 291685, 291686, 291687, 291688, 291689, 291690, 291691, 291692) c6( ) c7(T, id, dA, Uc, rd, if, WV, aA, cz, n, bB, v, fr, ci, qN, rP, fd, AO, q, ad, aHR, dP, aE, db, yt, aT, h, Ou, dL, ae, eL, cp, pl, atX, RT, s, qt, aW, Y, ff, r, aq, Ej, aF, en, cl, g, t, cm, bz, aHQ, iR, ck, es, bF, eM, nB, bE, gX, hL, m, aHP, y, it, B); #c1 (CD83) c2(NP_004224) c3(CD83 antigen isoform a precursor) c4(144424) c5(144425, 144426, 144427, 144428, 144429, 144430) c6( ) c7(T, dt, BD, FR, aEj, if, fd, hz, v, ame, ci, qN, Qp, V, q, cd, ad, wD, x, av, ib, aE, yt, aT, hD, h, lQ, Tj, KU, ae, Qe, iU, aAu, aW, Y, lh, lb, ev, eZ, ff, en, hf, g, t, cm, Zj, asE, Kc, sS, Bv, cj, k, m, Pk, bt, B, y); #c1(CD84) c2(NP_001171808) c3(SLAM family member 5 isoform 1 precursor) c4(7/784, 7/785) c5(7/786, 71787, 71788, 71789, 71790, 71791, 71792, 71793, 71794, 71795, 71796, 71797) c6( ) c7(If, y); #c1(CD84) c2(NP_001171810) c3(SLAM family member 5 isoform 3 precursor) c4(71805) c5(71806, 71807, 71808, 71809, 71810, 71811, 71812, 71813, 71814, 71815, 71816, 71817) c6( ) c7(If, y); #c1(CD84) c2(NP_001171811) c3(SLAM family member 5 isoform 4 precursor) c4(71818) c5(71819, 71820, 71821, 71822, 71823, 71824) c6( ) c7(lf, y); #c1(CD84) c2(NP_003865) c3(SLAM family member 5 isoform 2 precursor) c4(141614, 141615) c5(141616, 141617, 141618, 141619, 141620, 141621) c6( ) c7(If, y); #c1(CD84) c2(XP_011508396) c3(SLAM family member 5 isoform X1) c4(299098) c5(299099, 299100, 299101, 299102, 299103, 299104) c6( ) c7(If, y); #c1(CD86) c2(NP_001193853) c3(T-lymphocyte activation antigen CD86 isoform 4 precursor) c4(82731) c5(82732, 82733, 82734, 82735, 82736, 82737, 82738, 82739, 82740, 82741, 82742, 82743) c6( ) c7(Ob, gx, T, dD, fM, bM, FR, Dw, id, aHS, wi, axd, jx, jh, fd, n, aj, OY, j, ci, C, fu, eq, abC, V, q, Pm, J, hX, wa, eA, x, eW, wD, aE, mP, ds, aT, h, Ds, dL, ey, lQ, eL, ul, Nr, A, qZ, je, axu, Y, hl, eZ, la, r, aDn, lo, yq, po, fe, iV, cm, cM, pb, Wt, Nq, ck, cT, iM, eM, nB, yW, hr, cj, at, bk, k, y, bt, ne); #c1(CD86) c2(NP_001193854) c3(T-lymphocyte activation antigen CD86 isoform 5) c4(82744) c5(82745, 82746, 82747, 82748, 82749) c6( ) c7(Ob, gx, T, dD, fM, bM, FR, Dw, id, aHS, wi, axd, jx, jh, fd, n, aj, OY, j, ci, C, fu, eq, abC, V, q, Pm, J, hX, wa, eA, x, eW, wD, aE, mP, ds, aT, h, Ds, dL, ey, lQ, eL, ul, Nr, A, qZ, je, axu, Y, hl, eZ, la, r, aDn, lo, yq, po, fe, iV, cm, cM, pb, Wt, Nq, ck, cT, iM, eM, nB, yW, hr, cj, at, bk, k, y, bt, ne); #c1(CD86) c2(NP_008820) c3(T-lymphocyte activation antigen CD86 isoform 2 precursor) c4(168817, 168818) c5(168819, 168820, 168821, 168822, 168823, 168824) c6( ) c7(Ob, gx, T, dD, fM, bM, FR, Dw, id, aHS, wi, axd, jx, jh, fd, n, aj, OY, j, ci, C, fu, eq, abC, V, q, Pm, J, hX, wa, eA, x, eW, wD, aE, mP, ds, aT, h, Ds, dL, ey, lQ, eL, ul, Nr, A, qZ, je, axu, Y, hl, eZ, la, r, aDn, lo, yq, po, fe, iV, cm, cM, pb, Wt, Nq, ck, cT, iM, eM, nB, yW, hr, cj, at, bk, k, y, bt, ne); #c1(CD86) c2(NP_787058) c3(T-lymphocyte activation antigen CD86 isoform 1 precursor) c4(252039) c5(252040, 252041, 252042, 252043, 252044, 252045, 252046) c6( ) c7(Ob, gx, T, dD, fM, bM, fR, Dw, id, aHS, wi, axd, jx, jh, fd, n, aj, OY, j, ci, C, fu, eq, abC, V, q, Pm, J, hX, wa, eA, x, eW, wD, aE, mP, ds, aT, h, Ds, dl, ey, lQ, eL, ul, Nr, A, qZ, je, axu, Y, hl, eZ, la, r, aDn, lo, yq, po, fe, iV, cm, cM, pb, Wt, Nq, ck, cT, iM, eM, nB, yW, hr, cj, at, bk, k, y, bt, ne); #c1(CD86) c2(NP_795711) c3(T-lymphocyte activation antigen CD86 isoform 3 precursor) c4(252455) c5(252456, 252457, 252458, 252459, 252460, 252461) c6( ) c7(Ob, gx, T, dD, fM, bM, fR, Dw, id, aHS, wi, axd, jx, jh, fd, n, aj, OY, j, ci, C, fu, eq, abC, V, q, Pm, J, hX, wa, eA, x, eW, wD, aE, mP, ds, aT, h, Ds, dL, ey, lQ, eL, ul, Nr, A, qZ, je, axu, Y, hl, eZ, la, r, aDn, lo, yq, po, fe, iV, cm, cM, pb, Wt, Nq, ck, cT, iM, eM, nB, yW, hr, cj, at, bk, k, y, bt, ne); #c1(CD8A) c2(NP_001759) c3(T-cell surface glycoprotein CD8 alpha chain isoform 1 precursor) c4(120820, 120821, 120822, 120823) c5(120824, 120825, 120826, 120827, 120828, 120829, 120830, 120831, 120832, 120833, 120834, 120835, 120836, 120837, 120838, 120839, 120840, 120841, 120842, 120843, 120844, 120845, 120846, 120847, 120848, 120849, 120850, 120851) c6( ) c7(T, aHV, if, ht, eX, jh, aA, v, ci, V, aHT, ip, akU, qp, dV, aT, h, eV, iY, hu, lZ, aW, Y, eZ, aq, aHU, aF, t, f, cm, ik, ck, gX, cj, at, m, k, y, bt, B); #c1 (CD8A) c2(NP_741969) c3(T-cell surface glycoprotein CD8 alpha chain isoform 2 precursor) c4(247481) c5(247482, 247483, 247484, 247485, 247486, 247487, 247488, 247489, 247490, 247491) c6( ) c7(T, aHV, if, ht, eX, jh, aA, v, ci, V, aHT, ip, akU, qp, dV, aT, h, eV, iY, hu, lZ, aW, Y, eZ, aq, aHU, aF, t, f, cm, ik, ck, gX, cj, at, m, k, y, bt, B); #c1(CD8B) c2(NP_001171571) c3(T-cell surface glycoprotein CD8 beta chain isoform 6 precursor) c4(70639) c5(70640, 70641, 70642, 70643, 70644, 70645) c6( ) c7(ck, Pg, eZ, cj, jh, fl, act, mC); #c1(CD8B) c2(NP_004922) c3(T-cell surface glycoprotein CD8 beta chain isoform 5 precursor) c4(150808, 150809) c5(150810, 150811, 150812, 150813, 150814, 150815, 150816, 150817, 150818, 150819, 150820, 150821) c6( ) c7(ck, Pg, eZ, cj, jh, fl, act, mC); #c1(CD8B) c2(NP_742099) c3(T-cell surface glycoprotein CD8 beta chain isoform 3 precursor) c4(248088) c5(248089, 248090, 248091, 248092, 248093) c6( ) c7(ck, Pg, eZ, cj, jh, fl, act, mC); #c1(CD8B) c2(NP_742100) c3(T-cell surface glycoprotein CD8 beta chain isoform 4 precursor) c4(248094, 248095) c5(248096, 248097, 248098, 248099, 248100, 248101, 248102, 248103, 248104, 248105, 248106, 248107) c6( ) c7(ck, Pg, eZ, cj, jh, fl, act, mC); #c1(CD8B) c2(NP_757362) c3(T-cell surface glycoprotein CD8 beta chain isoform 2 precursor) c4(248703, 248704) c5(248705, 248706, 248707, 248708, 248709, 248710, 248711) c6( ) c7(ck, Pg, eZ, cj, jh, fl, act, mC); #c1 (CD9) c2(NP_001760) c3(CD9 antigen) c4(120852, 120853, 120854, 120855) c5(120856, 120857, 120858, 120859, 120860, 120861, 120862, 120863, 120864, 120865, 120866, 120867, 120868, 120869, 120870, 120871, 120872, 120873, 120874) c6( ) c7(T, dt, id, Uc, if, iW, aA, bB, n, fd, ci, qN, AO, ad, hX, ft, ds, jZ, HC, kH, aT, dV, SH, dL, h, he, nJ, ae, af, bc, CB, je, iU, aHW, aW, Y, Tr, kD, lo, aF, g, kk, cm, bz, ck, es, iM, iQ, cA, eM, bE, NL, cj, pg, aEG, Lb, zv, gn, aEN, bt, iu, EB); #c1(CD9) c2(XP_005253871) c3(CD9 antigen isoform X1) c4(272744) c5(272745, 272746, 272747, 272748, 272749, 272750) c6( ) c7(T, dt, id, Uc, if, iW, aA, bB, n, fd, ci, qN, AO, ad, hX, ft, ds, jZ, HC, kH, aT, dV, SH, dL, h, he, nJ, ae, af, bc, CB, je, iU, aHW, aW, Y, Tr, KD, lo, aF, g, kk, cm, bz, ck, es, iM, iQ, cA, eM, bE, NL, cj, pg, aEG, Lb, zv, gn, aEN, bt, iu, EB); #c1(CD9) c2(XP_011519342) c3(CD9 antigen isoform X2) c4( ) c5(313996, 313997, 313998, 313999, 314000, 314001, 314002, 314003, 314004, 314005, 314006) c6( ) c7(T, dt, id, Uc, if, iW, aA, bB, n, fd, ci, qN, AD, ad, hX, ft, ds, jZ, HC, KH, aT, dV, SH, dL, h, he, nJ, ae, af, bc, CB, je, iU, aHW, aW, Y, Tr, KD, lo, aF, g, kk, cm, bz, ck, es, iM, iQ, cA, eM, bE, NL, cj, pg, aEG, Lb, zv, gn, aEN, bt, iu, EB); #c1(CD93) c2(NP_036204) c3(complement component C1q receptor precursor) c4(172344, 172345) c5(172346, 172347, 172348, 172349, 172350, 172351, 172352, 172353, 172354, 172355, 172356, 172357) c6( ) c7(ac, W, ec, iW, aw, aT, dy, j, an); #c1(CD96) c2(NP_005807) c3(T-cell surface protein tactile isoform 2 precursor) c4(159610) c5(159611, 159612, 159613, 159614, 159615, 159616) c6( ) c7(ait, aHX, nX, Az, oA, atS, hF, bt, B); #c1(CD96) c2(NP_937839) c3(T-cell surface protein tactile isoform 1 precursor) c4(259272) c5(259273, 259274, 259275, 259276, 259277, 259278) c6( ) c7(ait, aHX, nX, Az, oA, atS, hF, bt, B); #c1(CD99) c2(NP_001116370) c3(CD99 antigen isoform b precursor) c4(43797) c5(43798, 43799, 43800, 43801, 43802, 43803) c6( ) c7(T, iH, afA, jh, ci, aHZ, iJ, ad, er, amR, ft, pX, ahj, SV, aT, dz, Ou, ahK, ae, bc, dx, jf, iF, JI, aW, oV, yx, eH, AQ, aF, g, Fu, eF, cm, adj, bw, asE, aHY, ck, EW, GU, eM, dB, apy, k, y, bt); #c1(CD99) c2(NP_002405) c3(CD99 antigen isoform a precursor) c4(128135, 128136) c5(128137, 128138, 128139, 128140, 128141, 128142) c6( ) c7(T, iH, afA, jh, ci, aHZ, iJ, ad, er, amR, ft, pX, ahj, SV, aT, dz, Ou, ahK, ae, bc, dx, jf, iF, JI, aW, oV, yx, eH, AQ, aF, g, Fu, eF, cm, adj, bw, asE, aHY, ck, EW, GU, eM, dB, apy, k, y, bt); #c1(CD99) c2(XP_005274851) c3(CD99 antigen isoform X1) c4(285491, 285492) c5(285493, 285494, 285495, 285496, 285497, 285498) c6( ) c7(T, iH, afA, jh, ci, aHZ, iJ, ad, er, amR, ft, pX, ahj, SV, aT, dz, Ou, ahK, ae, bc, dx, jf, iF, JI, aW, oV, yx, eH, AQ, aF, g, Fu, eF, cm, adj, bw, asE, aHY, ck, EW, GU, eM, dB, apy, k, y, bt); #c1(CD99) c2(XP_005274853) c3(CD99 antigen isoform X2) c4( ) c5(285499, 285500, 285501, 285502, 285503, 285504) c6( ) c7(T, iH, afA, jh, ci, aHZ, iJ, ad, er, amR, ft, pX, ahj, SV, aT, dz, Ou, ahK, ae, bc, dx, jf, iF, JI, aW, oV, yx, eH, AQ, aF, g, Fu, eF, cm, adj, bw, asE, aHY, ck, EW, GU, eM, dB, apy, k, y, bt); #c1(CD99) c2(XP_005274856) c3(CD99 antigen isoform X5) c4( ) c5(285505, 285506, 285507, 285508, 285509, 285510) c6( ) c7(T, iH, afA, jh, ci, aHZ, iJ, ad, er, amR, ft, pX, ahj, SV, aT, dz, Ou, ahK, ae, bc, dx, jf, iF, JI, aW, oV, yx, eH, AQ, aF, g, Fu, eF, cm, adj, bw, asE, aHY, ck, EW, GU, eM, dB, apy, k, y, bt); #c1(CDADC1) c2(NP_001180407) c3(cytidine and dCMP deaminase domain-containing protein 1 isoform 2) c4(73965) c5(73966, 73967, 73968, 73969, 73970, 73971) c6( ) c7(ce); #c1(CDADC1) c2(NP_112173) c3(cytidine and dCMP deaminase domain-containing protein 1 isoform 1) c4(220602, 220603) c5(220604, 220605, 220606, 220607, 220608, 220609) c6( ) c7(De); #c1 (CDADC1) c2(XP_006719934) c3(cytidine and dCMP deaminase domain-containing protein 1 isoform X5) c4( ) c5(293085) c6( ) c7(De); #c1(CDADC1) c2(XP_006719937) c3(cytidine and dCMP deaminase domain-containing protein 1 isoform X6) c4( ) c5(293086, 293087, 293088, 293089, 293090) c6( ) c7(oe); #c1(CDAN1) c2(NP_612486) c3(codanin-1) c4(236629) c5(236630, 236631, 236632, 236633, 236634, 236635, 236636) c6( ) c7(NU, Gq, ac, alc, alb, aBT, adw, lT, oW, fH, fe, ala, fC, aiS, BE, Bh, ald); #c1(CDAN1) c2(XP_005254233) c3(codanin-1 isoform X4) c4(273129) c5(273130, 273131, 273132, 273133, 273134, 273135) c6( ) c7(NU, Gq, ac, alc, alb, aBT, adw, lT, oW, fH, fe, ala, fC, aiS, BE, Bh, ald); #c1(CDAN1) c2(XP_011519572) c3(codanin-1 isoform X1) c4( ) c5(314237) c6( ) c7(NU, Gq, ac, alc, alb, aBT, adw, lT, oW, fH, fe, ala, fC, aiS, BE, Bh, ald); #c1(CDAN1) c2(XP_011519575) c3(codanin-1 isoform X5) c4( ) c5(314238, 314239, 314240) c6( ) c7(NU, Gq, ac, alc, alb, aBT, adw, lT, oW, fH, fe, ala, fC, aiS, BE, Bh, ald); #c1(CDC123) c2(NP_006014) c3(cell division cycle protein 123 homolog) c4(161166, 161167, 161168, 161169) c5(16/70, 16/171, 161172, 161173, 161174, 161175, 161176, 161177, 161178, 161179, 161180, 161181, 161182, 161183, 161184, 161185, 161186, 161187) c6( ) c7(bL, kh, aj, v, ale, qN, iJ, J, bv, aE, aT, h, ae, eL, dx, eG, Uf, d, aW, Y, eH, aq, aF, eF, bz, au, B); #c1(CDC(23) c2(XP_005252695) c3(cell division cycle protein 123 homolog isoform X1) c4(271993, 271994) c5(271995, 271996, 271997, 271998, 271999, 272000, 272001, 272002, 272003, 272004, 272005, 272006, 272007, 272008, 272009, 272010, 272011, 272012) c6( ) c7(bL, kh, aj, v, ale, qN, iJ, J, bv, aE, aT, h, ae, eL, dx, eG, Uf, d, aW, Y, eH, aq, aF, eF, bz, au, B); #c1(CDC14A) c2(XP_011540643) c3(dual specificity protein phosphatase CDC14A isoform X3) c4( ) c5(339179, 339180) c6( ) c7(aW, T, aA, aT); #c1(CDC14A) c2(XP_011540644) c3(dual specificity protein phosphatase CDC14A isoform X4) c4(339181) c5(339182, 339183, 339184, 339185, 339186, 339187) c6( ) c7(aW, T, aA, aT); #c1(CDC14A) c2(XP_011540646) c3(dual specificity protein phosphatase CDC14A isoform X7) c4( ) c5(339188, 339189, 339190, 339191, 339192) c6( ) c7(aW, T, aA, aT); #c1(CDC14A) c2(XP_011540648) c3(dual specificity protein phosphatase CDC14A isoform X9) c4( ) c5(339193, 339194, 339195, 339196, 339197, 339198) c6( ) c7(aW, T, aA, aT); #c1(CDC14B) c2(NP_001070649) c3(dual specificity protein phosphatase CDC14B isoform 3) c4(34651) c5( ) c6( ) c7(alf, n, py, d, as); #c1(CDC14B) c2(NP_003662) c3(dual specificity protein phosphatase CDC14B isoform 1) c4(140041) c5(140042, 140043, 140044, 140045, 140046, 140047) c6( ) c7(alf, n, py, d, as); #c1(CDC14B) c2(NP_201588) c3(dual specificity protein phosphatase CDC14B isoform 2) c4(228305, 228306) c5(228307) c6( ) c7(alf, n, py, d, as); #c1(CDC14B) c2(XP_011517453) c3(dual specificity protein phosphatase CDC14B isoform X5) c4( ) c5(311158, 311159, 311160, 311161, 311162, 311163, 311164, 311165, 311166, 311167) c6( ) c7(alf, n, py, d, as); #c1(CDC14B) c2(XP_011517457) c3(dual specificity protein phosphatase CDC14B isoform X9) c4( ) c5(311168, 311169, 311170, 311171, 311172) c6( ) c7(alf, n, py, d, as); #c1(CDC14B) c2(XP_011517459) c3(dual specificity protein phosphatase CDC14B isoform X11) c4( ) c5(311173, 311174, 311175, 311176, 311177, 311178) c6( ) c7(alf, n, py, d, as); #c1(CDC14B) c2(XP_011517460) c3(dual specificity protein phosphatase CDC14B isoform X12) c4(311179) c5(311180, 311181, 311182, 311183, 311184, 311185) c6( ) c7(alf, n, py, d, as); #c1(CDC16) c2(NP_003894) c3(cell division cycle protein 16 homolog) c4(141921) c5(141922, 141923, 141924, 141925, 141926, 141927, 141928, 141929, 141930, 141931) c6( ) c7(X, B, aT); #c1(CDC16) c2(XP_011533153) c3(cell division cycle protein 16 homolog isoform X2) c4(329300) c5(329301) c6( ) c7(X, B, aT); #c1(CDC20B) c2(NP_001139206) c3(cell division cycle protein 20 homolog B isoform 2) c4(58956, 58957) c5(58958, 58959, 58960, 58961, 58962, 58963, 58964, 58965, 58966, 58967, 58968) c6( ) c7(10); #c1(CDC20B) c2(NP_001163873) c3(cell division cycle protein 20 homolog B isoform 3) c4(66459) c5(66460, 66461, 66462, 66463, 66464, 66465, 66466) c6( ) c7(i0); #c1(CDC20B) c2(NP_689836) c3(cell division cycle protein 20 homolog B isoform 1) c4(244107) c5(244108, 244109, 244110, 244111, 244112, 244113) c6( ) c7(in); #c1(CDC20B) c2(XP_011541520) c3(cell division cycle protein 20 homolog B isoform X1) c4(340096) c5(340097, 340098, 340099, 340100, 340101, 340102) c6( ) c7(10); #c1(CDC23) c2(NP_004652) c3(cell division cycle protein 23 homolog) c4(148527, 148528) c5(148529, 148530, 148531, 148532, 148533, 148534, 148535, 148536, 148537, 148538, 148539, 148540) c6( ) c7(ck, T, hd, nM, iT, nB, X); #c1(CDC25A) c2(NP_001780) c3(M-phase inducer phosphatase 1 isoform a) c4(121043) c5(121044, 121045, 121046, 121047, 121048, 121049) c6( ) c7(T, gU, jh, aA, bB, ci, qN, q, ad, eA, cq, aE, arV, oW, aT, dL, K, ae, Qe, jf, zQ, s, aW, ff, hl, r, aq, aF, en, g, t, cm, ik, eM, bE, ayS, wB, at, m, bt, B); #c1(CDC25A) c2(NP_963861) c3(M-phase inducer phosphatase 1 isoform b) c4(262451) c5(262452, 262453, 262454, 262455, 262456, 262457) c6( ) c7(T, gU, jh, aA, bB, ci, qN, q, ad, eA, cq, aE, arV, oW, aT, dL, K, ae, Qe, jf, zQ, s, aW, ff, hl, r, aq, aF, en, g, t, cm, ik, eM, bE, ayS, wB, at, m, bt, B); #c1(CDC25A) c2(XP_006713499) c3(M-phase inducer phosphatase 1 isoform X4) c4(287170) c5(287171, 287172, 287173, 287/74, 287175, 287176) c6( ) c7(T, gU, jh, aA, bB, ci, qN, q, ad, eA, cq, aE, arV, oW, aT, dL, K, ae, de, jf, zQ, s, aW, ff, hl, r, aq, aF, en, g, t, cm, ik, eM, bE, ayS, wB, at, m, bt, B); #c1(CDC25B) c2(NP_001274445) c3(M-phase inducer phosphatase 2 isoform 4) c4(106541) c5(106542, 106543, 106544, 106545, 106546, 106547) c6( ) c7(T, ht, jh, ci, eA, ad, xy, aE, aT, dL, bK, K, hu, ae, d, ff, kp, en, t, bz, eM, hl, at, bt, it, B); #c1(CDC25B) c2(NP_001274447) c3(M-phase inducer phosphatase 2 isoform 6) c4(106548) c5(106549, 106550, 106551, 106552, 106553, 106554) c6( ) c7(T, ht, jh, ci, eA, ad, xy, aE, aT, dl, bk, K, hu, ae, d, ff, kp, en, t, bz, eM, hl, at, bt, it, B); #c1(CDC25B) c2(NP_004349) c3(M-phase inducer phosphatase 2 isoform 2) c4(145554) c5(145555, 145556, 145557, 145558, 145559, 145560) c6( ) c7(T, ht, jh, ci, eA, ad, xy, aE, aT, dL, bK, K, hu, ae, d, ff, kp, en, t, bz, eM, hl, at, bt, it, B); #c1 (CDC25B) c2(NP_068658) c3(M-phase inducer phosphatase 2 isoform 3) c4(209952) c5(209953, 209954, 209955, 209956, 209957, 209958) c6( ) c7(T, ht, jh, ci, eA, ad, xy, aE, aT, dL, bK, K, hu, ae, d, ff, kp, en, t, bz, eM, hl, at, bt, it, B); #c1(CDC25B) c2(NP_068659) c3(M-phase inducer phosphatase 2 isoform 1) c4(209959) c5(209960, 209961, 209962, 209963, 209964, 209965, 209966) c6( ) c7(T, ht, jh, ci, eA, ad, xy, aE, aT, dL, bK, K, hu, ae, d, ff, kp, en, t, bz, eM, hl, at, bt, it, B); #c1(CDC25C) c2(NP_001274511) c3(M-phase inducer phosphatase 3 isoform a) c4(106563, 106564) c5(106565, 106566, 106567, 106568, 106569, 106570, 106571, 106572, 106573, 106574, 106575, 106576, 106577, 106578, 106579) c6( ) c7(T, yS, zt, jh, aA, bB, n, fd, q, ad, eA, aE, dV, aT, h, ja, dL, K, ae, A, aW, ff, aq, af, en, cl, iR, t, bw, bz, ck, bE, wB, hL, cj, hR, at, m, B); #c1(CDC25C) c2(NP_001274512) c3(M-phase inducer phosphatase 3 isoform c) c4(106580) c5(106581, 106582, 106583, 106584, 106585, 106586, 106587, 106588, 106589, 106590) c6( ) c7(T, yS, zt, jh, aA, bB, n, fd, q, ad, eA, aE, dV, aT, h, ja, dL, K, ae, A, aW, ff, aq, aF, en, cl, iR, t, bw, bz, ck, bE, wB, hL, cj, hR, at, m, B); #c1(CDC25C) c2(NP_073720) c3(M-phase inducer phosphatase 3 isoform b) c4(213248) c5(213249, 213250, 213251, 213252, 213253, 213254, 213255, 213256) c6( ) c7(T, yS, zt, jh, aA, bB, n, fd, q, ad, eA, aE, dV, aT, h, ja, dL, K, ae, A, aW, ff, aq, af, en, cl, iR, t, bw, bz, ck, bE, wB, hL, cj, hR, at, m, B); #c1(CDC25C) c2(XP_011542061) c3(M-phase inducer phosphatase 3 isoform X3) c4(340588) c5(340589) c6( ) c7(T, yS, zt, jh, aA, bB, n, fd, q, ad, eA, aE, dV, aT, h, ja, dL, K, ae, A, aW, ff, aq, aF, en, cl, iR, t, bw, bz, ck, bE, wB, hL, cj, hR, at, m, B); #c1(CDC25C) c2(XP_011542063) c3(M-phase inducer phosphatase 3 isoform X6) c4( ) c5(340590, 340591) c6( ) c7(T, yS, zt, jh, aA, bB, n, fd, q, ad, eA, aE, dV, aT, h, ja, dL, K, ae, A, aW, ff, aq, af, en, cl, iR, t, bw, bz, ck, bE, wB, hL, cj, hR, at, m, B); #c1(CDC27) c2(NP_001107563) c3(cell division cycle protein 27 homolog isoform 1) c4(42858) c5(42859, 42860, 42861, 42862, 42863, 42864) c6( ) c7(aW, aT); #c1(CDC27) c2(NP_001280018) c3(cell division cycle protein 27 homolog isoform 3) c4(111258) c5(111259, 111260, 111261, 111262, 11/263, 111264) c6( ) c7(aW, aT); #c1(CDC27) c2(NP_001280020) c3(cell division cycle protein 27 homolog isoform 4) c4(111265) c5(111266, 111267, 111268, 111269, 111270, 111271) c6( ) c7(aW, aT); #c1(CDC34) c2(NP_004350) c3(ubiquitin-conjugating enzyme EZ RI) c4(145561) c5(145562, 145563, 145564, 145565, 145566, 145567) c6( ) c7(T, ht, alg, aT, hu, rP); #c1(CDC37) c2(NP_008996) c3(hsp90 co-chaperone Cdc37) c4(170134) c5(170135, 170136, 170137, 170138, 170139, 170140) c6( ) c7(T, azY, G, bE, cj, bB, fd, ci, K, MC, ae, B, ad); #c1 (CDC37LI) c2(NP_060383) c3(hsp90 co-chaperone Cdc37-like 1) c4(195722, 195723) c5(195724, 195725, 195726, 195727, 195728, 195729, 195730, 195731, 195732, 195733, 195734, 195735) c6( ) c7(ci); #c1(CDC42BPA) c2(NP_003598) c3(serine/threonine-protein kinase MRCK alpha isoform B) c4(139461, 139462) c5(139463, 139464, 139465, 139466, 139467, 139468, 139469, 139470, 139471, 139472, 139473, 139474, 139475, 139476, 139477, 139478, 139479, 139480) c6( ) c7(aBD, aC, aW, aBH, aT, nA, cd, dR); #c1(CDC42BPA) c2(NP_055641) c3(serine/threonine-protein kinase MRCK alpha isoform A) c4(182458) c5(182459, 182460, 182461, 182462, 182463, 182464) c6( ) c7(aBD, aC, aW, aBH, aT, nA, cd, dR); #c1(CDC42BPA) c2(XP_005273374) c3(serine/threonine-protein kinase MRCK alpha isoform X2) c4(284493, 284494) c5(284495, 284496, 284497, 284498, 284499, 284500, 284501, 284502, 284503, 284504, 284505) c6( ) c7(aBD, aC, aW, aBH, aT, nA, cd, dR); #c1(CDC42BPA) c2(XP_005273378) c3(serine/threonine-protein kinase MRCK alpha isoform X9) c4(284506, 284507) c5(284508, 284509, 284510, 284511, 284512, 284513, 284514, 284515, 284516, 284517, 284518, 284519) c6( ) c7(aBD, aC, aW, aBH, aT, nA, cd, dR); #c1 (CDC42BPA) c2(XP_005273379) c3(serine/threonine-protein kinase MRCK alpha isoform X11) c4(284520, 284521) c5(284522, 284523, 284524, 284525, 284526, 284527, 284528, 284529, 284530, 284531, 284532, 284533, 284534) c6( ) c7(aBD, aC, aW, aBH, aT, nA, cd, dR); #c1(CDC42BPA) c2(XP_005273381) c3(serine/threonine-protein kinase MRCK alpha isoform X12) c4(284535, 284536) c5(284537, 284538, 284539, 284540, 284541, 284542, 284543, 284544, 284545, 284546, 284547, 284548) c6( ) c7(aBD, aC, aW, aBH, aT, nA, cd, dR); #c1(CDC42BPA) c2(XP_011542610) c3(serine/threonine-protein kinase MRCK alpha isoform X8) c4(341180) c5( ) c6( ) c7(aBD, aC, aW, aBH, aT, nA, cd, dR); #c1(CDC42BPB) c2(NP_006026) c3(serine/threonine-protein kinase MRCK beta) c4(161253) c5(161254, 161255, 161256, 161257, 161258, 161259) c6( ) c7(dR, aW, T, jh, aT); #c1(CDC42BPB) c2(XP_005268286) c3(serine/threonine-protein kinase MRCK beta isoform X3) c4(281669) c5(281670, 281671, 281672, 281673, 281674, 281675) c6( ) c7(dR, aW, T, jh, aT); #c1(CDC42BPB) c2(XP_011535689) c3(serine/threonine-protein kinase MRCK beta isoform X5) c4( ) c5(332920) c6( ) c7(dR, aW, T, jh, aT); #c1(CDC42BPG) c2(NP_059995) c3(serine/threonine-protein kinase MRCK gamma) c4(193568) c5(193569, 193570, 193571, 193572, 193573, 193574) c6( ) c7(AW, aC); #c1(CDC42BPG) c2(XP_011543460) c3(serine/threonine-protein kinase MRCK gamma isoform X4) c4( ) c5(342356) c6( ) c7(AW, aC); #c1(CDC42EP1) c2(NP_689449) c3(cdc42 effector protein 1) c4(242740, 242741, 242742, 242743) c5(242744, 242745, 242746, 242747, 242748, 242749, 242750, 242751, 242752, 242753, 242754, 242755, 242756, 242757, 242758, 242759, 242760, 242761, 242762, 242763, 242764, 242765, 242766, 242767) c6( ) c7(y, eA); #c1(CDC42EP3) c2(NP_006440) c3(cdc42 effector protein 3) c4(165232, 165233, 165234, 165235) c5(165236, 165237, 165238, 165239, 165240, 165241, 165242, 165243, 165244, 165245, 165246, 165247, 165248, 165249, 165250, 165251, 165252, 165253, 165254, 165255, 165256, 165257) c6( ) c7(id, aj, j, g, Gp, bz, af, ad, iP); #c1(CDC42SE2) c2(NP_001033791) c3(CDC42 small effector protein 2) c4(31552) c5(31553, 31554, 31555, 31556, 31557, 31558, 31559, 31560, 31561, 31562, 31563, 31564, 31565, 31566) c6( ) c7(AM, oV, hL, c1); #c1(CDC45) c2(XP_005261342) c3(cell division control protein 45 homolog isoform X1) c4(277295, 277296) c5(277297, 277298, 277299, 277300, 277301, 277302) c6( ) c7(bz, RX, cj); #c1(CDC45) c2(XP_011528717) c3(cell division control protein 45 homolog isoform X3) c4(323986) c5(323987, 323988, 323989, 323990, 323991, 323992, 323993, 323994, 323995, 323996, 323997, 323998, 323999, 324000, 324001, 324002) c6( ) c7(bz, RX, cj); #c1(CDC45) c2(XP_011528720) c3(cell division control protein 45 homolog isoform X2) c4(324003) c5(324004, 324005, 324006, 324007, 324008, 324009) c6( ) c7(bz, RX, cj); #c1(CDC5L) c2(NP_001244) c3(cell division cycle 5-like protein) c4(92538) c5( ) c6( ) c7(eF, av, eH, aoM, c); #c1(CDC6) c2(NP_001245) c3(cell division control protein 6 homolog) c4(93495) c5( ) c6( ) c7(T, alh, if, iW, ci, bX, qN, ad, aoA, aT, ali, ae, kl, aW, ff, aq, en, QK, cj, at, B, cn); #c1(CDC7) c2(XP_011540528) c3(cell division cycle 7-related protein kinase isoform X1) c4(338996, 338997, 338998) c5(338999, 339000, 339001, 339002, 339003, 339004, 339005, 339006, 339007, 339008, 339009, 339010, 339011, 339012, 339013, 339014, 339015) c6( ) c7(T, BN, kP, c, dH, WY, Y, oe, aW, alR, ff, aT, aaE, hy, K, m, en, t, bt, fq, ne); #c1(CDC7) c2(XP_011540529) c3(cell division cycle 7-related protein kinase isoform X3) c4( ) c5(339016, 339017, 339018, 339019, 339020) c6( ) c7(T, BN, kP, c, dH, WY, Y, oe, aW, alR, ff, aT, aaE, hy, K, m, en, t, bt, fq, ne); #c1(CDC73) c2(NP_078805) c3(parafibromin) c4(215560) c5(215561, 215562, 215563, 215564, 215565, 215566) c6( ) c7(T, aC, pY, aw, n, fr, ci, ad, Do, yh, de, alk, ae, azh, bA, aAL, s, aAH, dg, HI, dr, es, YE, TG, iS, aru, azg, alj); #c1(CDC73) c2(XP_006711600) c3 (parafibromin isoform X1) c4(286177) c5(286178, 286179, 286180, 286181, 286182, 286183) c6( ) c7(T, aC, pY, aw, n, fr, ci, ad, Do, yh, dQ, alk, ae, azh, bA, aAL, s, aAH, dg, HI, dr, es, YE, TG, iS, aru, azg, alj); #c1(CDCA2) c2(NP_689775) c3(cell division cycle-associated protein 2) c4(243923, 243924) c5(243925, 243926, 243927, 243928, 243929, 243930) c6( ) c7(T, Y, aw, ff, en, bX); #c1(CDCA2) c2(XP_011542720) c3(cell division cycle-associated protein 2 isoform X2) c4(341288) c5( ) c6( ) c7(T, Y, aw, ff, en, bX); #c1(CDCA2) c2(XP_011542721) c3(cell division cycle-associated protein 2 isoform X4) c4( ) c5(341289, 341290, 341291, 341292, 341293) c6( ) c7(T, Y, aw, ff, en, bX); #c1(CDCA3) c2(NP_112589) c3(cell division cycle-associated protein 3 isoform 1) c4(221484) c5(221485, 221486, 221487, 221488, 221489, 221490, 221491, 221492, 221493, 221494, 221495, 221496, 221497) c6( ) c7(bL, T); #c1(CDCA5) c2(NP_542399) c3(sororin) c4(232337) c5(232338, 232339, 232340, 232341, 232342, 232343) c6( ) c7(T, hl, ci, aR, hX, hb, dV, af, A, Y, eH, Pg, eZ, as, eF, cm, bz, kT, WO, ac, WP, U, xg, ek); #c1(CDCA5) c2(XP_011543047) c3(sororin isoform X5) c4(341848) c5( ) c6( ) c7(T, hl, ci, aR, hX, hb, dV, af, A, Y, eH, Pg, eZ, as, eF, cm, bz, kT, WO, ac, WP, U, xg, ek); #c1(CDCA5) c2(XP_011543049) c3(sororin isoform X7) c4( ) c5(341849, 341850, 341851, 341852, 341853, 341854) c6( ) c7(T, hl, ci, aR, hX, hb, dV, af, A, Y, eH, Pg, eZ, as, eF, cm, bz, KT, WO, ac, WP, U, xg, ek); #c1(CDCA7) c2(NP_114148) c3(cell division cycle-associated protein 7 isoform 1) c4(222346) c5(222347, 222348, 222349, 222350, 222351, 222352) c6( ) c7(jf, T, W, iU, c, fq); #c1(CDCA7) c2(NP_665809) c3(cell division cycle-associated protein 7 isoform 2) c4(24/716, 241717) c5(241718, 24/719, 24/720, 24/721, 24/722, 241723, 24/724, 241725, 241726, 24/727, 24/728, 24/729) c6( ) c7(jf, T, W, iU, c, fq); #c1(CDCA7L) c2(NP_001120842) c3(cell division cycle-associated 7-like protein isoform 2) c4(44825, 44826) c5(44827, 44828, 44829, 44830, 44831, 44832, 44833, 44834, 44835, 44836) c6( ) c7(h, jf, B, an); #c1(CDCA7L) c2(NP_001120843) c3(cell division cycle-associated 7-like protein isoform 3) c4(44837) c5(44838, 44839, 44840, 44841, 44842, 44843) c6( ) c7(h, jf, B, an); #c1(CDCA7L) c2(NP_061189) c3(cell division cycle-associated 7-like protein isoform 1) c4(199892) c5(199893, 199894, 199895, 199896, 199897, 199898, 199899, 199900) c6( ) c7(h, jf, B, an); #c1(CDCP1) c2(NP_073753) c3(CUB domain-containing protein 1 isoform 1 precursor) c4(213466) c5(213467, 213468, 213469, 213470, 213471, 213472) c6( ) c7(T, aw, bB, n, fr, ci, q, ad, av, py, aE, aT, ey, dQ, ae, cY, iU, aW, r, aF, g, t, cM, bz, ck, bE, m, lB); #c1(CDCP1) c2(NP_835488) c3(CUB domain-containing protein 1 isoform 2 precursor) c4(253563) c5(253564, 253565, 253566, 253567, 253568, 253569) c6( ) c7(T, aw, bB, n, fr, ci, q, ad, av, py, aE, aT, ey, de, ae, cY, iU, aW, r, aF, g, t, cM, bz, ck, bE, m, lB); #c1(CDH1) c2(NP_004351) c3(cadherin-1 preproprotein) c4(145568, 145569, 145570) c5(145571, 145572, 145573, 145574, 145575, 145576, 145577, 145578, 145579, 145580, 145581, 145582, 145583, 145584, 145585, 145586, 145587, 145588) c6( ) c7(bM, T, aC, fR, pf, if, fd, bB, axk, ci, ip, Na, aE, ajv, ja, Ds, gs, hu, zy, eL, alo, YL, yo, zQ, aW, aEI, az, wf, nY, g, as, eF, ir, fT, eM, nB, iA, cj, m, alm, in, iu, aln, dD, Uc, QO, lT, sw, iB, wu, ht, n, fr, qN, pM, zP, Bl, jz, hw, gd, Gw, dL, hB, pl, YO, aq, kp, mo, cM, fs, op, dB, Qu, hL, hR, at, it, B, Dw, fM, jp, fG, aA, hy, AO, pd, q, er, eA, XX, ami, hW, Vr, eO, qK, HC, aT, AJ, Nc, ae, ne, jf, qw, avg, s, iU, Y, r, en, avb, cl, t, Px, iK, zJ, ck, nM, pZ, gX, aU, im, Kg, bt, hS, HQ, GI, vi, Mb, id, aD, iW, eq, Fc, iJ, ad, hX, dP, av, iC, hd, ib, py, db, yt, NS, wm, fb, BO, Ub, qt, ff, eH, aF, iV, iR, bz, YR, bF, es, apM, bE, AR, Gu, ra, X); #c1(CDH10) c2(NP_006718) c3(cadherin-10 preproprotein) c4(167392) c5(167393, 167394, 167395, 167396, 167397, 167398) c6( ) c7(bN, cd, j, ac, c); #c1(CDH11) c2(NP_001788) c3(cadherin-Il preproprotein) c4(121117, 121118, 121119) c5(121120, 121121, 121122, 121123, 121124, 121125, 121126, 121127, 121128, 121129, 121130, 121131, 121132) c6( ) c7(GI, T, fG, oe, ht, aw, bB, n, aj, hy, j, zh, ci, fr, cd, ad, ai, er, eA, afb, aT, Gw, ae, af, d, aW, fO, eH, as, eF, t, cM, fq, alp, LB, arY, acW, W, ac, zr, bE, iA, bP, Gu, zu, y); #c1(CDH13) c2(NP_001207417) c3(cadherin-13 isoform 2) c4(83647) c5(83648, 83649, 83650, 83651, 83652, 83653) c6( ) c7(T, pf, if, e, al, EQ, bB, ci, x, aE, dQ, xa, yo, aW, LZ, g, lk, eM, iT, m, k, aw, ht, n, fr, qN, alq, cd, h, hn, hB, cY, A, aq, bP, hL, hR, lB, it, B, fM, oe, aA, cz, v, j, q, eA, XX, GL, aT, ae, s, alr, iU, Y, r, iy, en, cl, t, ik, bN, ck, pH, kP, aps, bt, iW, jh, ad, av, Sb, na, d, ff, cr, af, iV, Z, bz, bF, gk, pc, Rm, ei, adg, y, aqD); #c1(CDH13) c2(NP_001207418) c3(cadherin-13 isoform 3 precursor) c4(83654, 83655) c5(83656, 83657, 83658, 83659, 83660, 83661) c6( ) c7(T, pf, if, e, al, EQ, bB, ci, x, aE, dQ, xa, yo, aW, LZ, g, lk, eM, iT, m, k, aw, ht, n, fr, qN, alq, cd, h, hn, hB, cY, A, aq, bP, hL, hR, IB, it, B, fM, oe, aA, cz, v, j, q, eA, XX, GL, aT, ae, s, alr, iU, Y, r, iy, en, cl, t, iK, bN, ck, pH, kP, aps, bt, iW, jh, ad, av, Sb, na, d, ff, cr, aF, lV, Z, bz, bF, gk, pc, Rm, ei, adg, y, aqD); #c1(CDH13) c2(NP_001207419) c3(cadherin-13 isoform 4) c4(83662, 83663) c5(83664, 83665, 83666, 83667, 83668, 83669, 83670, 83671, 83672, 83673, 83674, 83675, 83676, 83677, 83678, 83679, 83680, 83681) c6( ) c7(T, pf, if, e, al, EQ, bB, ci, x, aE, de, xa, yo, aW, LZ, g, lk, eM, iT, m, k, aw, ht, n, fr, qN, alq, cd, h, hn, hB, cY, A, aq, bP, hL, hR, lB, it, B, fM, oe, aA, cz, v, j, q, eA, XX, GL, aT, ae, s, alr, iU, Y, r, iy, en, cl, t, ik, bN, ck, pH, kP, aps, bt, iW, jh, ad, av, Sb, na, d, ff, cr, aF, iV, Z, bz, bF, gk, pc, Rm, ei, adg, y, aqD); #c1(CDH13) c2(NP_001207420) c3(cadherin-13 isoform 5 precursor) c4(83682) c5(83683, 83684, 83685, 83686, 83687, 83688, 83689) c6( ) c7(T, pf, if, e, al, EQ, bB, ci, x, aE, dQ, xa, yo, aW, LZ, g, lk, eM, iT, m, k, aw, ht, n, fr, qN, alq, cd, h, hn, hB, cY, A, aq, bP, hL, hR, lB, it, B, fM, oe, aA, cz, v, j, q, eA, XX, GL, aT, ae, s, alr, iU, Y, r, iy, en, cl, t, ik, bN, ck, pH, kP, aps, bt, iW, jh, ad, av, Sb, na, d, ff, cr, aF, iV, Z, bz, bF, gk, pc, Rm, ei, adg, y, aqD); #c1(CDH13) c2(NP_001207421) c3(cadherin-13 isoform 6 precursor) c4(83690) c5(83691, 83692, 83693, 83694, 83695, 83696, 83697, 83698, 83699) c6( ) c7(T, pf, if, e, al, EQ, bB, ci, x, aE, dQ, xa, yo, aW, LZ, g, lk, BM, iT, m, k, aw, ht, n, fr, qN, alq, cd, h, hn, hB, cY, A, aq, bP, hL, hR, lB, it, B, fM, oe, aA, cz, v, j, q, eA, XX, GL, aT, ae, s, alr, iU, Y, r, iy, en, c1, t, iK, bN, ck, pH, kP, aps, bt, iW, jh, ad, av, Sb, na, d, ff, cr, aF, iV, Z, bz, bF, gk, pc, Rm, ei, adg, y, aqD); #c1(CDH15) c2(NP_004924) c3(cadherin-15 preproprotein) c4(150822) c5(150823, 150824, 150825, 150826, 150827, 150828) c6( ) c7(T, fr, ci, nA, cd, q, alt, b, aE, arA, i, Sr, aT, qw, kl, aW, r, EZ, W, ac, alu, bo, als, m, WI); #c1(CDH16) c2(NP_001191673) c3(cadherin-16 isoform 2 precursor) c4(81585) c5(81586, 81587, 81588, 81589, 81590, 81591) c6( ) c7(es, hd, Uc, WA, rQ, fr, ci, ahv); #c1(CDH16) c2(NP_001191674) c3(cadherin-16 isoform 3 precursor) c4(81592) c5(81593, 81594, 81595, 81596, 81597, 81598) c6( ) c7(es, hd, Uc, WA, rl, fr, ci, ahv); #c1(CDH16) c2(NP_001191675) c3(cadherin-16 isoform 4 precursor) c4(81599) c5(81600, 81601, 81602, 81603, 81604, 81605) c6( ) c7(es, hd, Uc, WA, rQ, fr, ci, ahv); #c1(CDH16) c2(NP_004053) c3(cadherin-16 isoform 1 precursor) c4(143164, 143165) c5(143166, 143167, 143168, 143169, 143170, 143171, 143172, 143173, 143174, 143175, 143176, 143177) c6( ) c7(es, hd, Uc, WA, rQ, fr, ci, ahv); #c1(CDH17) c2(NP_001138135) c3(cadherin-17 precursor) c4(56042) c5(56043, 56044, 56045, 56046, 56047, 56048, 56049, 56050, 56051, 56052, 56053, 56054) c6( ) c7(T, pf, sw, by, aA, ci, q, ad, hX, hd, aE, alv, bK, ae, JI, Y, yL, ff, r, alw, aq, af, en, g, Z, t, iR, bz, bN, pH, bP, B, qc); #c1(CDH17) c2(XP_011515092) c3(cadherin-17 isoform X1) c4(307946) c5(307947, 307948, 307949, 307950, 307951, 307952) c6( ) c7(T, pf, sw, by, aA, ci, q, ad, hX, hd, aE, alv, bK, ae, JI, Y, yL, ff, r, alw, aq, aF, en, g, Z, t, iR, bz, bN, pH, bP, B, qc); #c1(CDH18) c2(NP_001161139) c3(cadherin-18 isoform 2 preproprotein) c4(65352) c5(65353, 65354, 65355, 65356, 65357, 65358) c6( ) c7(om, fq); #c1(CDH18) c2(NP_001278885) c3(cadherin-18 isoform 1 preproprotein) c4(110793) c5(110794, 110795, 110796, 110797, 110798, 110799, 110800, 110801, 110802, 110803, 110804, 110805, 110806, 110807, 110808, 110809, 110810, 110811) c6( ) c7(om, fq); #c1(CDH19) c2(NP_001257957) c3(cadherin-19 isoform 2 preproprotein) c4(95872) c5(95873, 95874, 95875, 95876, 95877, 95878) c6( ) c7(aw, b0); #c1(CDH19) c2(NP_066976) c3(cadherin-19 isoform 1 preproprotein) c4(207936, 207937) c5(207938, 207939, 207940, 207941, 207942, 207943) c6( ) c7(aw, b0); #c1(CDH19) c2(XP_011524234) c3(cadherin-19 isoform X2) c4( ) c5(318736, 318737, 318738, 318739, 318740, 318741) c6( ) c7(aw, b0); #c1(CDH2) c2(NP_001783) c3(cadherin-2 preproprotein) c4(121050) c5(121051, 121052, 121053, 121054, 121055, 121056) c6( ) c7(GI, T, aw, ht, aA, bB, fd, hy, v, j, ci, AD, cd, ad, eA, Na, iu, alv, HC, aT, Nc, dL, Gw, rE, ae, af, eL, YO, d, B, nO, aW, Y, yL, ff, en, g, bz, cA, eM, bE, mx, Gu, zl, cB, X, qc); #c1(CDH20) c2(XP_005266739) c3(cadherin-20 isoform X1) c4(280290) c5(280291, 280292, 280293, 280294, 280295, 280296, 280297, 280298, 280299, 280300, 280301, 280302, 280303, 280304) c6( ) c7(g, s, b0); #c1(CDH22) c2(XP_011527296) c3 (cadherin-22 isoform X1) c4(321919) c5(321920, 321921, 321922, 321923, 321924, 321925, 321926, 321927, 321928, 321929) c6( ) c7(bL); #c1(CDH23) c2(NP_001165401) c3(cadherin-23 isoform 3 precursor) c4(67653) c5( ) c6( ) c7(bM, pH, alx, alz, lR, aW, aw, alA, aly, aT, akl, nw, oc, nC, Bh, na); #c1(CDH23) c2(NP_001165402) c3(cadherin-23 isoform 4 precursor) c4(67654) c5( ) c6( ) c7(bM, pH, alx, alz, lR, aW, aw, alA, aly, aT, akl, nw, oc, nC, Bh, na); #c1(CDH23) c2(NP_071407) c3(cadherin-23 isoform 1 precursor) c4(211486) c5(211487, 211488, 211489, 211490, 211491, 211492, 211493, 211494, 211495, 211496, 211497, 211498, 211499, 211500, 211501, 211502, 211503) c6( ) c7(bM, pH, alx, alz, lR, aW, aw, alA, aly, aT, akl, nw, oc, nC, Bh, na); #c1(CDH23) c2(NP_443068) c3(cadherin-23 isoform 2 precursor) c4(229198, 229199, 229200) c5(229201, 229202, 229203, 229204, 229205, 229206, 229207, 229208, 229209, 229210, 229211, 229212, 229213, 229214, 229215, 229216) c6( ) c7(bM, pH, alx, alz, lR, aW, aw, alA, aly, aT, akl, nw, oc, nC, Bh, na); #c1(CDH23) c2(XP_006718005) c3(cadherin-23 isoform X5) c4(291426, 291427) c5(291428, 291429, 291430, 291431, 291432, 291433, 291434, 291435, 291436, 291437, 291438, 291439) c6( ) c7(bM, pH, alx, alz, lR, aW, aw, alA, aly, aT, akl, nw, oc, nC, Bh, na); #c1(CDH23) c2(XP_011538341) c3(cadherin-23 isoform X2) c4(336026) c5( ) c6( ) c7(bM, pH, alx, alz, lR, aW, aw, alA, aly, aT, akl, nw, oc, nC, Bh, na); #c1(CDH23) c2(XP_011538343) c3(cadherin-23 isoform X4) c4( ) c5(336027) c6( ) c7(bM, pH, alx, alz, lR, aW, aw, alA, aly, aT, akl, nw, oc, nC, Bh, na); #c1(CDH23) c2(XP_011538345) c3(cadherin-23 isoform X7) c4(336028) c5(336029, 336030, 336031, 336032, 336033, 336034) c6( ) c7(bM, pH, alx, alz, lR, aW, aw, alA, aly, aT, akl, nw, oc, nC, Bh, na); #c1(CDH23) c2(XP_011538348) c3(cadherin-23 isoform X10) c4(336035) c5( ) c6( ) c7(bM, pH, alx, alz, lR, aW, aw, alA, aly, aT, akl, nw, oc, nC, Bh, na); #c1(CDH23) c2(XP_011538355) c3(cadherin-23 isoform X16) c4( ) c5(336036, 336037, 336038, 336039, 336040, 336041) c6( ) c7(bM, pH, alx, alz, lR, aW, aw, alA, aly, aT, akl, nw, oc, nC, Bh, na); #c1(CDH23) c2(XP_011538356) c3(cadherin-23 isoform X17) c4( ) c5(336042, 336043, 336044, 336045, 336046, 336047, 336048, 336049) c6( ) c7(bM, pH, alx, alz, lR, aW, aw, alA, aly, aT, akl, nw, oc, nC, Bh, na); #c1(CDH26) c2(NP_068582) c3(cadherin-like protein 26 isoform b) c4(209710) c5(209711, 209712, 209713, 209714, 209715, 209716, 209717) c6( ) c7(bM, de); #c1(CDH26) c2(NP_817089) c3(cadherin-like protein 26 isoform a precursor) c4(253082) c5(253083, 253084, 253085, 253086, 253087, 253088) c6( ) c7(bM, oe); #c1(CDH26) c2(XP_006723910) c3(cadherin-like protein 26 isoform X5) c4(295441) c5(295442, 295443, 295444, 295445, 295446, 295447) c6( ) c7(bM, oe); #c1(CDH26) c2(XP_011527271) c3(cadherin-like protein 26 isoform X2) c4(321899, 321900) c5(321901, 321902, 321903, 321904, 321905, 321906) c6( ) c7(bM, oe); #c1(CDH26) c2(XP_011527272) c3(cadherin-like protein 26 isoform X3) c4( ) c5(321907, 321908, 321909, 321910, 321911, 321912) c6( ) c7(bM, oe); #c1(CDH3) c2(NP_001784) c3(cadherin-3 preproprotein) c4(121057, 121058) c5(121059, 121060, 121061, 121062, 121063, 121064, 121065) c6( ) c7(T, iB, aj, fr, ci, q, dP, aE, arA, Sr, aT, alB, alC, s, aW, Y, ff, r, en, g, iV, W, alu, als, m, hS); #c1(CDH4) c2(NP_001239267) c3(cadherin-4 isoform 2) c4( ) c5(88398, 88399, 88400, 88401, 88402, 88403) c6( ) c7(T, ac, gX, aA, aF, dy, ci); #c1(CDH4) c2(NP_001239268) c3(cadherin-4 isoform 3) c4(88404) c5( ) c6( ) c7(T, ac, gX, aA, aF, dy, ci); #c1(CDH4) c2(NP_001785) c3(cadherin-4 isoform 1 preproprotein) c4(121066, 121067) c5(121068, 121069, 121070, 121071, 121072, 121073, 121074, 121075, 121076, 121077, 121078, 121079) c6( ) c7(T, ac, gX, aA, af, dy, ci); #c1(CDH5) c2(NP_001786) c3(cadherin-5 preproprotein) c4(121080, 121081) c5(121082, 121083, 121084, 121085, 121086, 121087, 121088, 121089, 121090, 121091, 121092, 121093, 121094, 121095, 121096, 121097, 121098, 121099, 121100, 121101, 121102, 121103, 121104, 121105) c6( ) c7(alD, d, hd, ue, nB, bf, aW, Y, qK, mx, aT, cR, cm, dG, K, m, ml, ym, eA, bc); #c1(CDH5) c2(XP_011521103) c3(cadherin-5 isoform X1) c4(316032, 316033) c5( ) c6( ) c7(alD, d, hd, ue, nB, bf, aW, Y, qK, mx, aT, cR, cm, dG, K, m, ml, ym, eA, bc); #c1(CDH6) c2(XP_011512223) c3(cadherin-6 isoform X1) c4(302786) c5(302787, 302788, 302789, 302790, 302791, 302792) c6( ) c7(hd, iA, nB, aw, n, Qm, fr, ci, m, bz, t, fq, B); #c1(CDH7) c2(NP_387450) c3(cadherin-7 preproprotein) c4(228969) c5(228970, 228971, 228972, 228973, 228974, 228975, 228976, 228977, 228978, 228979, 228980, 228981, 228982, 228983) c6( ) c7(Y, bK, b0); #c1(CDH8) c2(NP_001787) c3(cadherin-8 preproprotein) c4(121106) c5(121107, 121108, 121109, 12/110, 121111, 121112, 121113, 121114, 121115, 121116) c6( ) c7(bN, aw, fr, an, rz, ao, xt); #c1(CDH9) c2(XP_011512224) c3(cadherin-9 isoform X1) c4(302793, 302794, 302795) c5(302796, 302797, 302798, 302799, 302800, 302801, 302802, 302803, 302804, 302805, 302806, 302807, 302808, 302809, 302810, 302811, 302812, 302813) c6( ) c7(pN, iX, oe, cz, aj, fr, Hb, rE, fq, bN, qc); #c1(CDHR1) c2(NP_001165442) c3(cadherin-related family member 1 isoform 2 precursor) c4(67727) c5(67728, 67729, 67730, 67731, 67732, 67733) c6( ) c7(alF, W, lR, nb, nx, nw, nC, alE); #c1(CDHR1) c2(NP_149091) c3(cadherin-related family member 1 isoform 1 precursor) c4(227080, 227081) c5(227082, 227083, 227084, 227085, 227086, 227087, 227088, 227089, 227090, 227091, 227092, 227093) c6( ) c7(alF, W, lR, nb, nx, nw, nC, alE); #c1(CDHR2) c2(NP_001165447) c3(cadherin-related family member 2 precursor) c4(67734) c5(67735, 67736, 67737, 67738, 67739, 67740, 67741, 67742, 67743, 67744, 67745, 67746, 67747, 67748) c6( ) c7(bB, bE, T, aq); #c1(CDHR3) c2(NP_689963) c3(cadherin-related family member 3 isoform 1 precursor) c4(244595, 244596) c5(244597, 244598, 244599, 244600, 244601, 244602, 244603, 244604, 244605, 244606, 244607, 244608) c6( ) c7(bM, hH, pM); #c1(CDHR3) c2(XP_011514257) c3(cadherin-related family member 3 isoform X2) c4(306824) c5( ) c6( ) c7(bM, hH, pM); #c1(CDHR3) c2(XP_011514260) c3(cadherin-related family member 3 isoform X7) c4(306825, 306826) c5(306827, 306828, 306829, 306830, 306831, 306832, 306833, 306834, 306835, 306836, 306837, 306838, 306839, 306840, 306841, 306842, 306843, 306844) c6( ) c7(bM, hH, pM); #c1(CDHR5) c2(NP_001165439) c3(cadherin-related family member 5 isoform 4 precursor) c4(67726) c5( ) c6( ) c7(aj, l, q, r); #c1(CDHR5) c2(NP_068743) c3(cadherin-related family member 5 isoform 1 precursor) c4(210137) c5(210138, 210139, 210140, 210141, 210142, 210143, 210144, 210145, 210146, 210147) c6( ) c7(aj, l, q, r); #c1(CDHR5) c2(NP_112554) c3(cadherin-related family member 5 isoform 3 precursor) c4(221374) c5(221375, 221376, 221377, 221378, 221379, 221380) c6( ) c7(aj, I, q, r); #c1(CDHR5) c2(XP_005253047) c3(cadherin-related family member 5 isoform X5) c4( ) c5(272114) c6( ) c7(aj, l, q, r); #c1(CDHR5) c2(XP_011518492) c3(cadherin-related family member 5 isoform X5) c4( ) c5(312562) c6( ) c7(aj, l, q, r); #c1(CDHR5) c2(XP_011518493) c3(cadherin-related family member 5 isoform X1) c4( ) c5(312563, 312564, 312565, 312566, 312567, 312568) c6( ) c7(aj, l, q, r); #c1(CDIPT) c2(NP_006310) c3(CDP-diacylglycerol-inositol 3-phosphatidyltransferase isoform 1) c4(163885, 163886) c5(163887, 163888, 163889, 163890, 163891, 163892, 163893, 163894, 163895, 163896, 163897, 163898) c6( ) c7(am, aek, N, alG); #c1(CDK1) c2(NP_203698) c3(cyclin-dependent kinase 1 isoform 2) c4(228457) c5(228458, 228459, 228460, 228461, 228462, 228463, 228464, 228465, 228466, 228467) c6( ) c7(T, os, hl, pf, zt, ha, jh, aA, bB, n, oZ, fd, j, ci, fK, q, ad, eA, dP, aE, aT, h, dL, K, jj, ae, af, eL, Lg, jf, d, nO, aW, Y, ff, r, aq, Xj, af, en, cl, t, ik, bz, ek, ck, bF, ac, eM, nB, bE, bP, do, U, hL, hR, np, m, bC, bt, KS, B, L); #c1(CDK1) c2(XP_006718145) c3(cyclin-dependent kinase 1 isoform X1) c4(291555, 291556, 291557) c5(291558, 291559, 291560, 291561, 291562, 291563, 291564, 291565, 291566, 291567, 291568, 291569, 291570, 291571, 291572, 291573, 291574, 291575) c6( ) c7(T, os, hl, pf, zt, ha, jh, aA, bB, n, oZ, fd, j, ci, fK, q, ad, eA, dP, aE, aT, h, dL, K, jj, ae, af, eL, Lg, jf, d, nO, aW, Y, ff, r, aq, Xj, aF, en, cl, t, ik, bz, ek, ck, bF, ac, eM, nB, bE, bP, d, U, hL, hR, np, m, bC, bt, KS, B, L); #c1(CDK10) c2(NP_443713) c3(cyclin-dependent kinase 10 isoform b) c4( ) c5(229950, 229951, 229952, 229953, 229954) c6( ) c7(T, os, aW, Y, aT, cd, oZ, B); #c1(CDK10) c2(NP_443714) c3(cyclin-dependent kinase 10 isoform a) c4(229955, 229956) c5(229957, 229958, 229959, 229960, 229961, 229962, 229963, 229964, 229965, 229966, 229967, 229968) c6( ) c7(T, os, aW, Y, aT, cd, oZ, B); #c1(CDK10) c2(XP_006721373) c3(cyclin-dependent kinase 10 isoform X7) c4(293953) c5(293954) c6( ) c7(T, os, aW, Y, aT, cd, oZ, B); #c1(CDK11B) c2(NP_001778) c3(cyclin-dependent kinase (IB isoform 1) c4(121026, 121027) c5(121028, 121029, (21030, 121031, 121032) c6( ) c7(asp, Y, au, eH, U, aT, eF, cm); #c1(CDK11B) c2(XP_011540794) c3(cyclin-dependent kinase 11B isoform X1) c4( ) c5(339354, 339355, 339356, 339357, 339358) c6( ) c7(asp, Y, au, eH, U, aT, eF, cm); #c1(CDK12) c2(NP_055898) c3(cyclin-dependent kinase 12 isoform 2) c4(184309) c5(184310, 184311, 184312, 184313, 184314, 184315) c6( ) c7(t, m); #c1(CDK12) c2(NP_057591) c3 (cyclin-dependent kinase 12 isoform 1) c4(191477) c5( ) c6( ) c7(t, m); #c1(CDK12) c2(XP_005257513) c3(cyclin- dependent kinase 12 isoform X1) c4( ) c5(275185, 275186, 275187, 275188, 275189, 275190, 275191) c6( ) c7(t, m); #c1(CDK13) c2(NP_003709) c3(cyclin-dependent kinase 13 isoform 1) c4(140337) c5(140338, 140339, 140340, 140341, 140342, 140343) c6( ) c7(aU, alH, q, r, B); #c1(CDK13) c2(NP_112557) c3(cyclin-dependent kinase 13 isoform 2) c4(221382) c5(221383, 221384, 221385, 221386, 221387, 221388) c6( ) c7(aU, aU, q, r, B); #c1(CDK13) c2(XP_011513900) c3(cyclin-dependent kinase 13 isoform X2) c4( ) c5(306335) c6( ) c7(aU, aU, q, r, B); #c1(CDK14) c2(NP_001274064) c3(cyclin-dependent kinase 14 isoform a) c4(105992) c5(105993, 105994, 105995, 105996, 105997, 105998) c6( ) c7(oe, B, ff, en); #c1(CDK14) c2(NP_001274066) c3(cyclin-dependent kinase 14 isoform d) c4(105999) c5(106000, 106001, 106002, 106003, 106004, 106005) c6( ) c7(oe, B, ff, en); #c1(CDK14) c2(NP_036527) c3(cyclin-dependent kinase 14 isoform b) c4(174232) c5(174233, 174234, 174235, 174236, 174237, 174238) c6( ) c7(oe, B, ff, en); #c1(CDK15) c2(NP_001248364) c3(cyclin-dependent kinase 15 isoform 1) c4(93809) c5(93810, 93811, 93812, 93813, 93814, 93815) c6( ) c7(T, Y, alJ, K, MC, eW); #c1(CDK15) c2(NP_001248365) c3(cyclin-dependent kinase 15 isoform 2) c4(93816) c5(93817, 93818, 93819, 93820, 93821, 93822) c6( ) c7(T, Y, alJ, K, MC, eW); #c1(CDK15) c2(NP_631897) c3(cyclin-dependent kinase 15 isoform 3) c4( ) c5(238256, 238257, 238258, 238259, 238260, 238261, 238262, 238263) c6( ) c7(T, Y, alJ, K, MC, eW); #c1(CDK15) c2(XP_006712759) c3(cyclin-dependent kinase 15 isoform X2) c4(286822, 286823) c5(286824, 286825, 286826, 286827, 286828, 286829, 286830, 286831) c6( ) c7(T, Y, alJ, K, MC, eW); #c1(CDK16) c2(NP_001163931) c3(cyclin-dependent kinase 16 isoform 3) c4(66508, 66509) c5(66510, 66511, 66512, 66513, 66514, 66515, 66516) c6( ) c7(T, lR, Y, alJ, MC, K, oQ, QK, eW); #c1(CDK16) c2(NP_006192) c3(cyclin-dependent kinase 16 isoform 1) c4( ) c5(162747, 162748, 162749, 162750, 162751, 162752, 162753, 162754, 162755, 162756, 162757, 162758, 162759, 162760, 162761, 162762, 162763, 162764, 162765, 162766, 162767, 162768, 162769, 162770, 162771) c6( ) c7(T, lR, Y, alJ, MC, K, oQ, QK, eW); #c1(CDK16) c2(NP_148978) c3(cyclin-dependent kinase 16 isoform 2) c4(226668, 226669, 226670, 226671, 226672, 226673, 226674) c5(226675, 226676, 226677, 226678, 226679, 226680, 226681, 226682, 226683, 226684, 226685, 226686, 226687, 226688, 226689, 226690, 226691, 226692, 226693, 226694, 226695, 226696, 226697) c6( ) c7(T, lR, Y, alJ, MC, K, oQ, QK, eW); #c1(CDK16) c2(XP_011542223) c3(cyclin-dependent kinase 16 isoform X2) c4( ) c5(340724) c6( ) c7(T, lR, Y, alJ, MC, K, oQ, QK, eW); #c1(CDK16) c2(XP_011542224) c3(cyclin-dependent kinase 16 isoform X3) c4( ) c5(340725, 340726) c6( ) c7(T, lR, Y, alJ, MC, K, oQ, QK, eW); #c1(CDK17) c2(NP_002586) c3(cyclin-dependent kinase 17 isoform 1) c4(130080) c5(130081, 130082, 130083, 130084, 130085, 130086) c6( ) c7(Ew, an); #c1(CDK17) c2(XP_011536737) c3(cyclin-dependent kinase 17 isoform X1) c4(334369) c5(334370, 334371, 334372, 334373, 334374, 334375) c6( ) c7(Ew, an); #c1(CDK17) c2(XP_011536738) c3(cyclin-dependent kinase 17 isoform X2) c4(334376, 334377, 334378) c5(334379, 334380, 334381, 334382, 334383, 334384, 334385, 334386, 334387, 334388, 334389, 334390, 334391, 334392, 334393, 334394, 334395, 334396) c6( ) c7(Ew, an); #c1(CDK18) c2(NP_002587) c3(cyclin-dependent kinase 18 isoform b) c4( ) c5(130087, 130088, 130089, 130090, 130091, 130092) c6( ) c7(eA); #c1(CDK18) c2(NP_997668) c3(cyclin-dependent kinase 18 isoform a) c4(265675, 265676) c5(265677, 265678, 265679, 265680, 265681, 265682, 265683, 265684, 265685, 265686, 265687, 265688) c6( ) c7(eA); #c1(CDK18) c2(XP_011507904) c3(cyclin-dependent kinase 18 isoform X1) c4(298181, 298182) c5(298183, 298184, 298185, 298186, 298187, 298188, 298189, 298190, 298191, 298192) c6( ) c7(eA); #c1(CDK19) c2(NP_001287892) c3(cyclin-dependent kinase 19 isoform 3) c4( ) c5(113150, 113151, 113152, 113153, 113154, 113155, 113156, 113157, 113158, 113159, 113160, 113161, 113162, 113163, 113164, 113165) c6( ) c7(au, vA, eH, r, eF, q); #c1(CDK19) c2(NP_055891) c3(cyclin-dependent kinase 19 isoform 1) c4(184217, 184218, 184219, 184220) c5(184221, 184222, 184223, 184224, 184225, 184226, 184227, 184228) c6( ) c7(au, vA, eH, r, eF, q); #c1(CDK19) c2(XP_005266928) c3(cyclin-dependent kinase 19 isoform X4) c4(280406) c5(280407, 280408, 280409, 280410, 280411, 280412) c6( ) c7(au, vA, eH, r, eF, q); #c1(CDK2) c2(NP_001789) c3(cyclin-dependent kinase 2 isoform 1) c4(121133) c5(121134, 121135, 121136, 121137, 121138, 121139) c6( ) c7(bM, T, aC, if, fd, bB, ci, ip, x, aE, dE, ajv, K, hu, eL, xQ, JI, aW, nY, g, alk, as, eF, cm, iP, eM, nB, iA, vA, cj, PV, m, k, sc, wu, ht, qy, awr, qN, ft, h, dL, A, kj, fQ, aq, op, bP, dB, azr, it, B, fM, dA, aA, pd, q, eA, Vr, qK, aT, vH, ae, jf, qw, s, vT, iU, Y, r, lo, en, cl, t, iK, ck, iM, Dy, DW, wR, np, bt, hS, kh, id, aHA, jh, OE, Fc, ad, hd, F, ib, Rq, wm, fb, Yi, d, je, ff, eH, aF, iR, bz, es, Hw, bE, afd, bC, y, X, jr); #c1(CDK2) c2(NP_439892) c3(cyclin-dependent kinase 2 isoform 2) c4(229130) c5(229131, 229132, 229133, 229134, 229135, 229136) c6( ) c7(bM, T, aC, if, fd, bB, ci, ip, x, aE, dE, ajv, K, hu, eL, xQ, JI, aW, nY, g, alk, as, eF, cm, iP, eM, nB, iA, vA, cj, PV, m, k, sc, wu, ht, qy, awr, qN, ft, h, dL, A, kj, fQ, aq, op, bP, dB, azr, it, B, fM, dA, aA, pd, q, eA, Vr, qK, aT, vH, ae, jf, qw, s, vT, iU, Y, r, lo, en, cl, t, ik, ck, iM, Dy, DW, wR, np, bt, hS, kh, id, aHA, jh, OE, Fc, ad, hd, F, ib, Rq, wm, fb, Yi, d, je, ff, eH, aF, iR, bz, es, Hw, bE, afd, bC, y, X, jr); #c1(CDK20) c2(NP_001034892) c3(cyclin-dependent kinase 20 isoform 3) c4(32383) c5(32384, 32385, 32386, 32387, 32388, 32389) c6( ) c7(T, d, yw, kB, h, m, hH); #c1(CDK20) c2(NP_001164111) c3(cyclin-dependent kinase 20 isoform 5) c4(66629) c5(66630, 66631, 66632, 66633, 66634, 66635) c6( ) c7(T, d, yw, kB, h, m, hH); #c1(CDK20) c2(NP_036251) c3(cyclin-dependent kinase 20 isoform 2) c4(172641, 172642) c5(172643, 172644, 172645, 172646, 172647, 172648, 172649, 172650, 172651, 172652, 172653, 172654) c6( ) c7(T, d, yw, kB, h, m, hH); #c1(CDK20) c2(NP_848519) c3(cyclin-dependent kinase 20 isoform 1) c4(253760) c5(253761, 253762, 253763, 253764, 253765, 253766) c6( ) c7(T, d, yw, kB, h, m, hH); #c1(CDK2AP1) c2(NP_004633) c3(cyclin-dependent kinase 2-associated protein 1 isoform 1) c4(148382) c5(148383, 148384, 148385, 148386, 148387, 148388) c6( ) c7(T, ff, r, ey, ci, m, en, t, V, ae, q, ad); #c1(CDK2AP2) c2(NP_005842) c3(cyclin-dependent kinase 2-associated protein 2 isoform 1) c4(159806) c5(159807, 159808, 159809, 159810, 159811, 159812) c6( ) c7(sc, jf, T, ft, aEj, aW, Y, jh, aT, cj, r, dL, aF, ci, K, q, EY, g, fr, B, ad); #c1(CDK3) c2(NP_001249) c3(cyclin-dependent kinase 3) c4(94024) c5(94025, 94026, 94027, 94028, 94029, 94030, 94031, 94032, 94033, 94034) c6( ) c7(T, d, if, iw, qN, B); #c1(CDK4) c2(NP_000066) c3(cyclin-dependent kinase 4) c4(1124, 1125, 1126, 1127, 1128, 1129) c5(1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165) c6( ) c7(G, T, pf, hV, YU, if, alN, fd, bB, axk, ci, ip, x, bv, aE, u, pX, SV, K, eL, avq, de, eG, yo, zQ, aW, hl, CT, Qm, g, as, eF, cm, QA, iP, eM, cj, k, m, Xu, iu, alM, sc, iB, qy, n, aj, kv, fr, qN, zP, pe, aeX, Lv, dL, dx, Lg, A, kj, iw, ks, fl, aq, kp, iN, bJ, bP, dB, hk, eD, hL, hj, at, it, B, bL, fM, yk, fZ, aA, hy, v, q, J, er, eA, lg, hW, qK, yg, aT, als, cy, ae, af, jf, iF, qw, s, vT, Or, nO, Y, iU, r, en, hf, bu, t, cl, ik, ck, ov, QG, kP, Dy, ac, au, aU, bt, hS, On, kh, iW, jh, KO, hF, Fc, ad, afb, av, l, hd, ib, vJ, hP, fb, DI, d, BN, ff, eH, bz, ek, bF, bE, QC, y, ne); #c1(CDK5) c2(NP_001157882) c3(cyclin-dependent-like kinase 5 isoform 2) c4(62858) c5(62859, 62860, 62861, 62862, 62863, 62864) c6( ) c7(bL, T, dD, ea, fd, bB, GC, aj, eq, bX, aR, ad, hd, aE, nv, hb, M, aT, aU, cy, dL, K, an, ae, af, eu, Lg, aii, xX, kl, aW, eH, cr, as, eF, cm, bz, FT, ck, ok, rR, WO, c, nB, ac, bE, eM, bP, U, at, act, ei, xg, gD, ek); #c1(CDK5) c2(NP_004926) c3(cyclin-dependent-like kinase 5 isoform 1) c4(150829) c5(150830, 150831, 150832, 150833, 150834, 150835) c6( ) c7(bL, T, dD, ea, fd, bB, GC, aj, eq, bX, aR, ad, hd, aE, nv, hb, M, aT, aU, cy, dL, K, an, ae, af, eu, Lg, aii, xX, kl, aW, eH, cr, as, eF, cm, bz, FT, ck, ok, rR, WO, c, nB, ac, bE, eM, bP, U, at, act, ei, xg, gD, ek); #c1(CDK5R1) c2(XP_011523740) c3 (cyclin-dependent kinase 5 activator 1 isoform X1) c4(318212) c5(318213, 318214, 318215, 318216, 318217, 318218) c6( ) c7(T, hl, aU, fr, nA, alP, alR, aR, ad, hX, Hx, hb, dV, aT, h, K, ae, af, Lg, eG, A, Y, eH, Pg, eZ, as, eF, cm, kT, WO, c, ac, bP, WP, U, auT, xg, gD, ek); #c1(CDK5R2) c2(NP_003927) c3(cyclin-dependent kinase 5 activator 2) c4(142233) c5(142234, 142235, 142236, 142237, 142238, 142239) c6( ) c7(hb, af, Pg); #c1(CDK5RAP1) c2(NP_001265096) c3(CDK5 regulatory subunit-associated protein 1 isoform c) c4( ) c5(97773, 97774, 97775, 97776, 97777, 97778) c6( ) c7(cF); #c1(CDK5RAP1) c2(NP_001265097) c3(CDK5 regulatory subunit-associated protein 1 isoform d) c4(97779, 97780) c5(97781, 97782, 97783, 97784, 97785, 97786, 97787) c6( ) c7(cF); #c1(CDK5RAP1) c2(NP_057166) c3(CDK5 regulatory subunit-associated protein 1 isoform b) c4( ) c5(189273) c6( ) c7(cF); #c1(CDK5RAP1) c2(NP_057492) c3(CDK5 regulatory subunit-associated protein 1 isoform a) c4(191157, 191158, 191159, 19/160) c5(191161, 191162, 191163, 191164, 191165, 191166, 191167, 191168, 191169, 191170, 191171, 19/172, 19/173, 191174, 191175, 191176, 191177) c6( ) c7(cF); #c1(CDK5RAP1) c2(XP_011527157) c3(CDK5 regulatory subunit-associated protein 1 isoform X1) c4( ) c5(321674, 321675, 321676, 321677, 321678) c6( ) c7(cF); #c1(CDK5RAP2) c2(NP_001011649) c3(CDK5 regulatory subunit-associated protein 2 isoform b) c4(24661) c5(24662, 24663, 24664, 24665, 24666, 24667) c6( ) c7(QK, aii, aij, alS); #c1(CDK5RAP2) c2(NP_001258968) c3(CDK5 regulatory subunit-associated protein 2 isoform c) c4(96841) c5(96842, 96843, 96844, 96845, 96846, 96847) c6( ) c7(QK, aii, aij, alS); #c1(CDK5RAP2) c2(NP_060719) c3(CDK5 regulatory subunit-associated protein 2 isoform a) c4(197461, 197462) c5(197463, 197464, 197465, 197466, 197467, 197468, 197469, 197470, 197471, 197472, 197473, 197474) c6( ) c7(QK, aii, aij, alS); #c1(CDK5RAP2) c2(XP_006717245) c3(CDK5 regulatory subunit-associated protein 2 isoform X3) c4(290820) c5(290821, 290822, 290823, 290824, 290825, 290826) c6( ) c7(QK, aii, aij, alS); #c1(CDK5RAP3) c2(NP_788276) c3(CDK5 regulatory subunit-associated protein 3 isoform b) c4(252174) c5(252175, 252176, 252177, 252178, 252179, 252180) c6( ) c7(T, B, bE, ev, X, r, bB, K, g, q, oU); #c1(CDK5RAP3) c2(XP_011523599) c3(CDK5 regulatory subunit-associated protein 3 isoform X1) c4(318078) c5(318079, 318080, 318081, 318082, 318083, 318084) c6( ) c7(T, B, bE, ev, X, r, bB, K, g, q, oU); #c1(CDK6) c2(XP_006715898) c3(cyclin-dependent kinase 6 isoform X1) c4(289892) c5(289893, 289894, 289895, 289896, 289897, 289898, 289899, 289900, 289901, 289902) c6( ) c7(sc, T, fM, pf, wu, jh, aA, it, fd, cl, aon, eA, o, ad, x, BI, aE, pX, aT, h, dL, wm, fb, ae, eL, aij, jf, A, d, aW, Y, ff, hl, kp, aF, yq, en, hf, g, t, ve, bz, pb, cm, iP, ck, Dy, eM, nB, bP, hL, cj, NI, m, k, y, bt, xb, B); #c1(CDK7) c2(NP_001790) c3(cyclin-dependent kinase 7) c4(121140) c5(121141, 121142, 121143, 121144, 121145, 121146) c6( ) c7(bL, T, ci, bX, V, eA, ad, av, ft, aE, NS, aT, dL, ae, d, aW, nY, t, bz, bJ, ck, at, m, jQ, X); #c1(CDK8) c2(NP_001251) c3(cyclin-dependent kinase 8) c4( ) c5( ) c6( ) c7(T, bE, Y, vA, eH, NT, gd, r, bB, hL, h, fr, Ea, cl, eF, q); #c1(CDK9) c2(NP_001252) c3(cyclin-dependent kinase 9) c4( ) c5( ) c6( ) c7(Dw, T, aC, alT, jh, C, cd, ad, iu, cc, ds, aT, cU, h, K, bq, ae, dx, cp, aa, jf, iF, d, aW, aq, lo, as, cm, ck, eM, bt, avU, B); #c1(CDKAL1) c2(NP_060244) c3(threonylcarbamoyladenosine tRNA methylthiotransferase) c4(194960) c5(194961, 194962, 194963, 194964, 194965, 194966, 194967, 194968, 194969, 194970) c6( ) c7(bL, T, dD, aZ, ea, aw, aj, dG, j, v, eq, cd, q, J, mj, h, dL, eL, sj, kl, A, zR, r, cr, fe, iV, fq, ac, mg, mf, lS, ei); #c1(CDKAL1) c2(XP_011513020) c3(threonylcarbamoyladenosine tRNA methylthiotransferase isoform X1) c4(304629) c5(304630, 304631, 304632, 304633, 304634, 304635) c6( ) c7(bL, T, dD, aZ, ea, aw, aj, dG, j, v, eq, cd, q, J, mj, h, dL, eL, sj, kl, A, zR, r, cr, fe, iV, fq, ac, mg, mf, lS, ei); #c1(CDKL1) c2(NP_004187) c3(cyclin-dependent kinase-like 1 isoform 1) c4(144/70) c5(144171, 144172, 144173, 144174, 144175, 144176) c6( ) c7(T, yw, kB, aA, h, aF); #c1 (CDKL2) c2(NP_003939) c3(cyclin-dependent kinase-like 2) c4(142339) c5(142340, 142341, 142342, 142343, 142344, 142345) c6( ) c7(aCp, Be); #c1(CDKL3) c2(NP_001107047) c3(cyclin-dependent kinase-like 3 isoform 1) c4(42807) c5(42808, 42809, 42810, 42811, 42812, 42813) c6( ) c7(bw, nA); #c1(CDKL3) c2(NP_057592) c3(cyclin-dependent kinase-like 3 isoform 2) c4( ) c5(191478, 191479, 191480, 191481, 191482, 191483, 191484, 191485, 191486, 191487) c6( ) c7(bw, nA); #c1(CDKL3) c2(XP_006714708) c3(cyclin-dependent kinase-like 3 isoform X4) c4( ) c5(288033, 288034, 288035, 288036, 288037, 288038) c6( ) c7(bw, nA); #c1(CDKL3) c2(XP_011541732) c3(cyclin-dependent kinase-like 3 isoform X1) c4( ) c5(340337) c6( ) c7(bw, nA); #c1(CDKL3) c2(XP_011541733) c3(cyclin-dependent kinase-like 3 isoform X2) c4(340338, 340339) c5( ) c6( ) c7(bw, nA); #c1(CDKL3) c2(XP_011541734) c3(cyclin-dependent kinase-like 3 isoform X5) c4(340340) c5(340341, 340342, 340343, 340344, 340345, 340346) c6( ) c7(bw, nA); #c1(CDKL3) c2(XP_011541749) c3(cyclin-dependent kinase-like 3 isoform X12) c4(340347) c5(340348, 340349, 340350, 340351, 340352) c6( ) c7(bw, nA); #c1(CDKL4) c2(NP_001009565) c3(cyclin-dependent kinase-like 4) c4(23618) c5(23619, 23620, 23621, 23622, 23623, 23624) c6( ) c7(li, agl); #c1(CDKL5) c2(NP_001032420) c3(cyclin-dependent kinase-like 5) c4(31340) c5(31341, 31342, 31343, 31344, 31345, 31346, 31347, 31348, 31349, 31350) c6( ) c7(ha, Nt, nA, aR, alY, hb, alX, lm, he, alV, cC, yr, alW, alX, ahm, nf, QK, f, dr, bN, alU, Qi, WO, mG, agl); #c1(CDKN1A) c2(NP_000380) c3(cyclin-dependent kinase inhibitor 1 isoform 1) c4(8316) c5(8317, 8318, 8319, 8320, 8321, 8322, 8323, 8324, 8325, 8326, 8327) c6( ) c7(gh, T, os, bM, aC, pf, ea, if, al, fd, bB, ci, aJa, ip, x, bv, afr, aE, aEX, Qk, ajv, K, hu, eL, bc, de, aa, yo, Jl, aW, wF, hl, CT, g, as, pl, eE, eF, cm, aAB, eM, nB, AM, iA, iT, vA, wE, cj, k, m, jM, iu, dD, afA, Or, aw, ht, NT, n, alZ, fr, Qh, qN, asS, ai, eW, co, Bl, zw, hw, lt, kH, Lv, lG, aEo, dL, h, bl, eV, Gw, alb, pl, aua, ks, fe, aq, kp, Hu, YF, dw, cM, Ld, Zj, op, Ye, bP, dB, ln, hL, pV, hR, rx, at, fL, WL, it, B, fM, AS, jp, yk, uv, aoE, jx, eX, aA, sM, bZ, hy, v, atp, q, eA, eJ, MR, lg, akU, Vr, ahl, aT, kf, cy, Fx, jj, yb, bq, ae, ne, Lu, ls, wr, s, NE, iU, Y, ev, aEk, Lf, r, en, bu, cl, t, f, ik, ck, QG, nM, iM, pZ, ll, nG, agf, np, dy, im, bt, rA, hS, HQ, GI, Mb, kh, id, pY, iW, jh, Hn, oZ, dG, eq, pz, aFJ, iJ, mC, ad, hX, dP, av, Tg, hd, ib, Rq, oW, ahk, fb, ul, DI, d, je, qt, vj, ff, eH, cr, eZ, Ej, gE, VI, aF, iV, iR, KM, bz, Hp, LB, bF, gk, bE, bC, Gu, y, kS, X, rk); #c1(CDKN1A) c2(NP_001278478) c3(cyclin-dependent kinase inhibitor 1 isoform 2) c4(109756) c5(109757, 109758, 109759, 109760, 109761, 109762, 109763, 109764, 109765, 109766) c6( ) c7(gh, T, os, bM, aC, pf, ea, if, al, fd, bB, ci, aJa, ip, x, bv, afr, aE, aEX, Qk, ajv, K, hu, eL, bc, de, aa, yo, JI, aW, wf, hl, CT, g, as, pl, eE, eF, cm, aAB, eM, nB, AM, iA, iT, vA, wE, cj, k, m, jM, iu, dD, afA, Or, aw, ht, NT, n, alZ, fr, Qh, qN, asS, ai, eW, co, Bl, zw, hw, lt, KH, Lv, lG, aEo, dL, h, bl, eV, Gw, aJb, pl, aua, ks, fe, aq, kp, Hu, YF, dw, cM, Ld, Zj, op, Ye, bP, dB, ln, hL, pV, hR, rx, at, fL, WL, it, B, fM, AS, jp, yk, uv, aDE, jx, eX, aA, sM, bZ, hy, v, atp, q, eA, eJ, MR, lg, akU, Vr, ahL, aT, kf, cy, Fx, jj, yb, bq, ae, ne, Lu, ls, wr, s, NE, iU, Y, ev, aEk, Lf, r, en, bu, cl, t, f, ik, ck, QG, nM, iM, pZ, ll, nG, agf, np, dy, im, bt, rA, hS, HQ, GI, Mb, kh, id, pY, iW, jh, Hn, oZ, dG, eq, pz, aFJ, iJ, mC, ad, hX, dP, av, Tg, hd, ib, Rq, oW, ahK, fb, ul, DI, d, je, qt, vj, ff, eH, cr, eZ, Ej, gE, VI, aF, iV, iR, KM, bz, Hp, LB, bF, gk, bE, bC, Gu, y, kS, X, rk); #c1 (CDKN1B) c2(NP_004055) c3(cyclin-dependent kinase inhibitor (B) c4(143178, 143179) c5(143180, 143181, 143182, 143183, 143184, 143185, 143186, 143187, 143188, 143189, 143190, 143191) c6( ) c7(gh, G, T, bM, aC, ea, if, fd, bB, ci, x, Vp, Na, aE, il, pX, Qk, K, hu, eL, Qe, yo, aeR, iE, aW, az, nY, Qv, g, as, eF, cm, alg, bw, iP, qJ, ou, fT, eM, nB, iA, alh, iT, cj, NI, iS, cX, azg, k, m, OD, iu, cZ, dD, afA, lT, aw, ht, NT, n, aj, fr, qN, zP, JW, eW, aJe, aJf, ft, jz, hw, Do, Bj, KH, Ne, lG, h, dL, ey, eV, dd, pl, Oz, kj, aJd, AQ, fe, aq, yq, PU, dw, cM, SE, zZ, op, iO, KT, YE, bP, dB, Fi, eD, PI, hL, hR, E, at, HB, avr, qF, anx, B, cn, fM, jx, eX, aA, cz, hy, j, atp, q, J, eA, zn, aHy, nv, Vr, aT, j, ae, af, jf, s, NE, nO, iU, Y, r, iy, lo, eS, en, hf, cl, t, f, ck, rR, nM, ac, iM, DW, nG, bt, aJc, hS, id, pY, iW, YM, jh, DE, Fb, eq, Fc, iJ, ad, hX, dP, fU, av, hd, ib, py, db, ds, NS, oW, hP, nJ, d, BN, je, Ub, vj, ff, eH, aF, VI, HI, bz, es, RV, bE, aS, aed, ne); #c1(CDKN1C) c2(NP_000067) c3(cyclin-dependent kinase inhibitor IC isoform a) c4(1166, 1167) c5(1168, 1169, 1170, 1171, 1172, 1173, 1174, 1175, 1176, 1177, 1178, 1179, 1180) c6( ) c7(T, aC, ea, ci, x, bv, Na, aE, aX, iv, K, hu, eL, ZH, aW, uY, g, cm, bw, eM, nB, k, m, ali, dD, aw, ht, fg, aj, rc, br, aoc, kH, jy, hn, dl, ey, CN, iw, aq, LI, Hu, bJ, iO, bP, hL, at, eo, B, bL, ul, fM, AL, aA, V, q, J, er, eA, mj, aT, ae, s, Or, Y, LO, r, en, t, ik, ck, alC, iM, dy, bt, pY, iW, ml, eq, ad, Jm, av, fX, ib, nJ, iL, gj, d, je, ff, Lk, aF, bz, aJj, dr); #c1(CDKN1C) c2(NP_001116103) c3(cyclin-dependent kinase inhibitor IC isoform b) c4( ) c5(43419, 43420, 43421, 43422, 43423, 43424, 43425, 43426) c6( ) c7(T, aC, ea, ci, x, bv, Na, aE, aX, iv, K, hu, eL, ZH, aW, uY, g, cm, bw, eM, nB, k, m, ali, dD, aw, ht, fg, aj, rc, br, aoc, kH, jy, hn, dl, ey, CN, iw, aq, LI, Hu, bJ, iO, bP, hL, at, eo, B, bL, ul, fM, AL, aA, V, q, J, er, eA, mj, aT, ae, s, Or, Y, LO, r, en, t, iK, ck, alC, iM, dy, bt, pY, iW, ml, eq, ad, Jm, av, fX, ib, nJ, iL, gj, d, je, ff, Lk, aF, bz, adj, dr); #c1(CDKN2A) c2(NP_000068) c3(cyclin-dependent kinase inhibitor 2A isoform p16INK4a) c4(1181) c5(1182, 1183, 1184, 1185, 1186, 1187, 1188, 1189) c6( ) c7(T, ea, bO, fd, aeu, aJN, ci, YD, bv, aE, ajv, auB, SV, iv, hu, hH, cp, aa, li, yo, Ls, az, QB, CT, nY, iA, Jr, nB, avZ, cj, cX, k, lS, fy, YK, aln, iB, ht, aj, br, aex, KN, Gw, Dz, asH, aEL, YT, pl, ge, ahJ, pm, hq, iO, dB, eD, aAE, aJC, aJJ, fZ, aA, cz, bX, AO, aJs, q, zn, akU, qK, yu, rO, jj, aJA, amw, eu, if, bf, NE, nO, lo, eS, cl, rR, iM, ac, nG, WU, aor, aps, bt, rA, On, Mb, aEd, iW, jh, Hn, KO, dG, Sv, Gn, ad, afb, hX, iC, oW, lQ, wm, apw, DI, BO, ff, gK, Of, aeV, alv, iV, bz, aux, amS, aJm, lC, ne, rk, jr, BA, iu, bM, Fv, if, e, bB, bh, aon, ye, Tz, ip, aJO, x, Ma, aeI, ja, aJu, bc, avq, YL, xQ, eG, auQ, aeR, ahG, JI, dH, fc, hl, kt, Qm, as, eF, cm, bw, qJ, atU, vA, iS, CD, OD, ik, Vj, sc, dD, Qj, wu, aDT, n, uE, aJr, b, no, pe, ec, jz, eV, hn, A, kj, AQ, hQ, GA, aJP, bP, hL, at, pg, SZ, B, AS, dA, eX, v, Qo, V, GS, eJ, hW, wN, BJ, eO, AJ, ae, jf, Lu, iZ, vT, auc, aJD, aJq, bu, apx, LV, t, ck, kP, au, gX, vi, nZ, hl, pY, eq, Fc, aGW, hd, aop, db, hD, aJR, YQ, auY, d, je, zR, aJk, anW, Z, adj, bF, gk, Ny, afd, pP, gZ, rH, y, aJV, atT, aC, FR, hV, atj, al, alN, Jt, afr, alp, Xc, rJ, pX, Qk, M, K, eL, aJE, aEI, aoo, mQ, XV, LZ, hN, g, pl, iP, aAW, aJX, ou, alw, aJH, U, PV, m, QO, sw, zX, aw, NT, fN, qy, zP, ft, lt, Nn, kH, dx, aoX, aJL, aJG, ks, Og, fs, Zj, ar, mg, azr, zl, WL, it, fM, aDE, jx, j, pd, eA, J, er, XX, MR, lg, aAF, aJn, xR, Y, ayU, r, en, ik, QG, Dy, c, aJS, dy, EY, GI, kh, id, aHA, bS, OH, hF, iJ, dP, ajk, io, hP, Yi, BN, anv, eH, cr, aJK, aJW, aJI, iR, Ew, AR, bC, hE, tQ, pf, WV, aJM, aJo, MO, pk, Zm, ayT, akV, zQ, aAu, aW, ark, arZ, eM, iT, aep, avo, zv, jM, Xu, qR, OF, aEj, Or, aBx, Bm, Wb, awr, fr, ct, qN, ai, eW, axC, Bl, hw, h, dL, hB, SD, Lg, wC, iw, aq, kp, Hu, mo, dw, op, oU, Qy, Qu, hk, Vy, hR, uz, oh, anx, cn, bl, yk, WT, hy, mj, Vr, aoA, afQ, aT, aly, dz, an, aJB, af, aol, qw, s, iU, aU, gm, hf, Vq, aJU, sS, nM, alt, aJY, aJd, ij, hS, aD, aJF, OH, aFJ, av, F, ib, Rq, fb, iL, yF, UT, qt, eZ, auP, Ej, aF, aJT, Hp, LB, es, auJ, eU, bE, aJz, aS, MC, adg, X, us); #c1(CDKN2A) c2(NP_001182061) c3 (cyclin-dependent kinase inhibitor ZA isoform p16gamma) c4(74528) c5(74529, 74530, 74531, 74532, 74533, 74534, 74535, 74536, 74537, 74538) c6( ) c7(T, ea, bO, fd, aeu, aJN, ci, YD, bv, aE, ajv, auB, SV, iv, hu, hH, cp, aa, li, yo, Ls, az, QB, CT, nY, iA, Jr, nB, avZ, cj, cX, k, lS, fy, YK, aln, iB, ht, aj, br, aex, KN, Gw, Dz, asH, aEL, YT, pl, ge, ahJ, pm, hq, iO, dB, eD, aAE, aJC, aJJ, fZ, aA, cz, bX, AO, aJs, q, zn, akU, qK, yu, rO, jj, aJA, amw, eu, iF, bf, NE, nO, lo, eS, cl, rR, iM, ac, nG, WU, aor, aps, bt, rA, On, Mb, aEd, iW, jh, Hn, KO, dG, Sv, Gn, ad, afb, hX, iC, oW, lQ, wm, apw, DI, BO, ff, gk, Of, aeV, aJv, iV, bz, alx, amS, aJm, lC, ne, rk, jr, BA, iu, bM, Fv, if, e, bB, bh, aon, ye, Tz, ip, aJO, x, Ma, aeO, ja, aJu, bc, avq, YL, xQ, eG, auQ, aeR, ahG, JI, dH, fc, hl, kt, Qm, as, eF, cm, bw, qJ, atU, vA, iS, CD, OD, ik, Vj, sc, dD, Qj, wu, aDT, n, uE, aJr, b, no, pe, ec, jz, eV, hn, A, kj, AQ, hQ, GA, aJP, bP, hL, at, pg, SZ, B, AS, dA, eX, v, Qo, V, GS, eJ, hW, wN, BJ, eO, AJ, ae, jf, Lu, iZ, vT, auc, aJD, alq, bu, apx, LV, t, ck, kP, au, gX, vi, nZ, hl, pY, eq, Fc, aGW, hd, aop, db, hD, aJR, YQ, auY, d, je, zR, aJk, anW, Z, adj, bF, gk, Ny, afd, pP, gZ, rH, y, aJV, atT, aC, fR, hV, atj, al, alN, Jt, afr, alp, Xc, rJ, pX, Qk, M, K, eL, aJE, aEI, aoo, mQ, XV, LZ, hN, g, pl, iP, aAW, aJX, ou, aJw, aJH, U, PV, m, oO, sw, zX, aw, NT, fN, qy, zP, ft, lt, Nn, kH, dx, aoX, aJL, aJG, ks, Og, fs, Zj, ar, mg, azr, zl, WL, it, fM, aDE, jx, j, pd, eA, J, er, XX, MR, lg, aAF, aJn, xR, Y, ayU, r, en, ik, QG, Dy, c, aJS, dy, EY, Gl, kh, id, aHA, bS, OH, hF, iJ, dP, ajk, io, hP, Yi, BN, anv, eH, cr, aJK, aJW, aU, iR, Ew, AR, bC, hE, tQ, pf, WV, aJM, aJo, MO, pk, Zm, ayT, akV, zQ, aAu, aW, ark, arZ, eM, iT, aep, avo, zv, jM, Xu, qR, OF, aEj, Or, aBx, Bm, Wb, awr, fr, ct, qN, ai, eW, axC, Bl, hw, h, dL, hB, SD, Lg, wC, iw, aq, kp, Hu, mo, dw, op, oU, Qy, Qu, hk, Vy, hR, uz, oh, anx, cn, bL, yk, WT, hy, mj, Vr, aoA, afQ, aT, aly, dz, an, aJB, af, aol, qw, s, iU, aU, gm, hf, Vq, aJU, sS, nM, alt, aJY, aJQ, ij, hS, aD, aJF, OH, aFJ, av, F, ib, Rq, fb, iL, yF, UT, qt, eZ, auP, Ej, aF, aJT, Hp, LB, es, auJ, eU, bE, alz, aS, MC, adg, X, us); #c1(CDKN2A) c2(NP_478102) c3(cyclin-dependent kinase inhibitor ZA isoform p14ARF) c4(230894, 230895) c5(230896, 230897, 230898, 230899, 230900) c6( ) c7(T, ea, bO, fd, aeu, aJN, ci, YD, bv, aE, ajv, auB, SV, iv, hu, hH, cp, aa, li, yo, Ls, az, QB, CT, nY, iA, Jr, nB, avZ, cj, cX, k, lS, fy, YK, aln, iB, ht, aj, br, aex, KN, Gw, Dz, asH, aEL, YT, pl, ge, ahJ, pm, hq, iO, dB, eD, aAE, aJC, aJJ, fZ, aA, cz, bX, AO, aJs, q, zn, akU, qK, yu, rO, jj, aJA, amw, eu, if, bf, NE, nO, lo, eS, cl, rR, iM, ac, nG, WU, aor, aps, bt, rA, On, Mb, aEd, iW, jh, Hn, KO, dG, Sv, Gn, ad, afb, hX, iC, oW, lQ, wm, apw, DI, BO, ff, gK, Of, aeV, alv, iV, bz, alx, amS, aJm, lC, ne, rk, jr, BA, iu, bM, Fv, if, e, bB, bh, aon, ye, Tz, ip, aJO, x, Ma, aeO, ja, aJu, bc, avq, YL, xQ, eG, auQ, aeR, ahG, JI, dH, fc, hl, kt, Qm, as, eF, cm, bw, qJ, atU, vA, iS, CD, OD, ik, Vj, sc, dD, Qj, wu, aDT, n, uE, aJr, b, no, pe, ec, jz, eV, hn, A, kj, AL, hQ, GA, aJP, bP, hL, at, pg, SZ, B, AS, dA, eX, v, Qo, V, GS, eJ, hW, wN, BJ, eO, AJ, ae, jf, Lu, iZ, vT, auc, aJD, alq, bu, apx, LV, t, ck, kP, au, gX, vi, nZ, hl, pY, eq, Fc, aGW, hd, aop, db, hD, aJR, YQ, auY, d, je, zR, aJk, anW, Z, adj, bF, gk, Ny, afd, pP, gZ, rH, y, aJV, atT, aC, fR, hV, atj, al, alN, Jt, afr, alp, Xc, rJ, pX, Qk, M, K, eL, aJE, aEI, aoo, mQ, XV, LZ, hN, g, pl, iP, aAW, aJX, ou, aJw, aJH, U, PV, m, oO, sw, zX, aw, NT, fN, qy, zP, ft, lt, Nn, kH, dx, aoX, aJL, aJG, ks, Og, fs, Zj, ar, mg, azr, zl, WL, it, fM, aDE, jx, j, pd, eA, J, er, XX, MR, lg, aAF, aJn, xR, Y, ayU, r, en, ik, QG, Dy, c, aJS, dy, EY, Gl, kh, id, aHA, bS, OH, hF, iJ, dP, ajk, io, hP, Yi, BN, anv, eH, cr, aJK, aJW, aU, iR, Ew, AR, bC, hE, tQ, pf, WV, aJM, aJo, MO, pk, Zm, ayT, akV, zQ, aAu, aW, ark, arZ, eM, iT, aep, avo, zv, jM, Xu, qR, OF, aEj, Or, aBx, Bm, Wb, awr, fr, ct, qN, ai, eW, axC, Bl, hw, h, dL, hB, SD, Lg, wC, iw, aq, kp, Hu, mo, dw, op, oU, Qy, Qu, hk, Vy, hR, uz, oh, anx, cn, bL, yk, WT, hy, mj, Vr, aoA, afd, aT, aly, dz, an, aJB, af, aol, qw, s, iU, aU, gm, hf, Vq, aJU, sS, nM, alt, aJY, aJQ, ij, hS, aD, aJF, OH, aFJ, av, F, ib, Rq, fb, iL, yF, UT, qt, eZ, auP, Ej, aF, aJT, Hp, LB, es, auJ, eU, bE, aJz, aS, MC, adg, X, us); #c1(CDKN2A) c2(XP_011515979) c3(cyclin-dependent kinase inhibitor 2A isoform X3) c4( ) c5(309150, 309151, 309152, 309153, 309154) c6( ) c7(T, ea, bO, fd, aeu, aJN, ci, YD, bv, aE, ajv, auB, SV, iv, hu, hH, cp, aa, li, yo, Ls, az, QB, CT, nY, iA, Jr, nB, avZ, cj, cX, k, lS, fy, YK, aln, iB, ht, aj, br, aex, KN, Gw, Dz, asH, aEL, YT, pl, ge, ahJ, pm, hq, iO, dB, eD, aAE, aJC, aJJ, fZ, aA, cz, bX, AO, aJs, q, zn, akU, qK, yu, r, jj, aJA, amw, eu, iF, bf, NE, nO, lo, eS, cl, rR, iM, ac, nG, WU, aor, aps, bt, rA, On, Mb, aEd, iW, jh, Hn, KO, dG, Sv, Gn, ad, afb, hX, iC, oW, lQ, wm, apw, DI, BO, ff, gK, Of, aeV, alv, iV, bz, alx, amS, alm, lC, ne, rk, jr, BA, iu, bM, Fv, if, e, bB, bh, aon, ye, Tz, ip, aJO, x, Ma, aeO, ja, aJu, bc, avq, YL, xQ, eG, auQ, aeR, ahG, JI, dH, fc, hl, kt, Qm, as, eF, cm, bw, qJ, atU, vA, iS, CD, OD, ik, Vj, sc, dD, Qj, wu, aDT, n, uE, aJr, b, no, pe, ec, jz, eV, hn, A, kj, AQ, hQ, GA, aJP, bP, hL, at, pg, SZ, B, AS, dA, eX, v, Qo, V, GS, eJ, hW, wN, BJ, eO, AJ, ae, jf, Lu, iZ, vT, auc, aJD, aJq, bu, apx, LV, t, ck, kP, au, gX, vi, nZ, hl, pY, eq, Fc, aGW, hd, aop, db, hD, aJR, YQ, auY, d, je, zR, aJk, anW, Z, adj, bF, gk, Ny, afd, pP, gZ, rH, y, aJV, atT, aC, fR, hV, atj, al, alN, Jt, afr, adp, Xc, rJ, pX, Qk, M, K, eL, aJE, aEI, aoo, mQ, XV, LZ, hN, g, pl, iP, aAW, aJX, ou, alw, aJH, U, PV, m, oO, sw, zX, aw, NT, fN, qy, zP, ft, lt, Nn, kH, dx, aoX, aJL, aJG, ks, Og, fs, Zj, ar, mg, azr, zl, WL, it, fM, aoE, jx, j, pd, eA, J, er, XX, MR, lg, aAF, aJn, xR, Y, ayU, r, en, ik, QG, Dy, c, aJS, dy, EY, GI, kh, id, aHA, bS, OH, hF, iJ, dP, ajk, io, hP, Yi, BN, anv, eH, cr, aJK, aJW, aU, iR, Ew, AR, bC, hE, tQ, pf, WV, aJM, aJo, MO, pk, Zm, ayT, akV, zQ, aAu, aW, ark, arZ, eM, iT, aep, avo, zv, jM, Xu, qR, OF, aEj, Or, aBx, Bm, Wb, awr, fr, ct, qN, ai, eW, axC, Bl, hw, h, dL, hB, SD, Lg, wC, iw, aq, kp, Hu, mo, dw, op, oU, By, Qu, hk, Vy, hR, uz, oh, anx, cn, bL, yk, WT, hy, mj, Vr, aoA, afQ, aT, aly, dz, an, aJB, af, aol, qw, s, iU, aU, gm, hf, Vq, aJU, sS, nM, alt, aJY, aJA, ij, hS, aD, aJF, OH, aFJ, av, F, ib, Rq, fb, iL, yF, UT, qt, eZ, auP, Ej, aF, aJT, Hp, LB, es, auJ, eU, bE, aJz, aS, MC, adg, X, us); #c1(CDKN2AIP) c2(NP_060102) c3(CDKN2A-interacting protein) c4(194133) c5(194134, 194135, 194136, 194137, 194138, 194139) c6( ) c7(T, K); #c1(CDKN2B) c2(NP_004927) c3(cyclin-dependent kinase 4 inhibitor B isoform 1) c4(150836) c5(150837, 150838, 150839, 150840, 150841, 150842, 150843) c6( ) c7(bM, T, aC, tQ, ea, e, qx, al, fd, bB, ci, Tz, ip, x, bv, aE, aeI, M, K, hu, pk, eL, Zm, xQ, yo, aeR, dH, JI, aW, mQ, nY, g, as, eF, cm, bw, iP, qJ, ou, eM, nB, iT, cj, PV, CD, k, m, lS, vF, ik, sc, cZ, dD, aEj, Or, aw, ht, fN, qy, n, aj, oY, fr, qN, br, ft, ec, KH, h, hn, dl, dd, A, aix, ks, dw, oU, iO, bP, dB, hk, kz, Fi, mg, hL, pg, uz, it, B, bL, fM, AS, jx, aA, cz, v, j, GS, als, q, J, eA, zn, eJ, mj, lg, nv, afQ, aT, dz, jj, XZ, ae, eu, aol, jf, bf, s, nO, iU, xR, Y, r, en, cl, t, ik, ck, cT, sS, iM, Dy, ac, pZ, gX, aor, bt, vi, HQ, hS, kh, id, pY, aHA, iW, jh, bs, dG, eq, hF, Fc, ad, iC, av, hd, fX, ib, db, aun, hP, fb, pq, iL, d, je, ff, zR, aJK, Ej, aF, bz, bF, gk, eU, Ew, amS, aJm, AR, gZ, aS, y, iu, us); #c1(CDKN2B) c2(NP_511042) c3(cyclin-dependent kinase 4 inhibitor B isoform 2) c4(231122) c5(231123, 231124, 231125, 231126, 231127, 231128, 231129, 231130, 231131) c6( ) c7(bM, T, aC, tl, ea, e, qx, al, fd, bB, ci, Tz, ip, x, bv, aE, aeI, M, K, hu, pk, eL, Zm, xQ, yo, aeR, dH, JI, aW, md, nY, g, as, eF, cm, bw, iP, qJ, ou, eM, nB, iT, cj, PV, CD, k, m, lS, vF, ik, sc, cZ, dD, aEj, Or, aw, ht, fN, qy, n, aj, oY, fr, qN, br, ft, ec, kH, h, hn, dL, dd, A, aix, ks, dw, oU, iO, bP, dB, hk, kz, Fi, mg, hl, pg, uz, it, B, bL, fM, AS, jx, aA, cz, v, j, GS, aJs, q, J, eA, zn, eJ, mj, lg, nv, afQ, aT, dz, jj, XZ, ae, eu, aol, jf, bf, s, nO, iU, xR, Y, r, en, cl, t, iK, ck, cT, sS, iM, Dy, ac, pZ, gX, aor, bt, vi, HQ, hS, kh, id, pY, aHA, iW, jh, bs, dG, eq, hF, Fc, ad, iC, av, hd, fX, ib, db, aun, hP, fb, pq, iL, d, je, ff, zR, aJK, Ej, aF, bz, bF, gk, eU, Ew, amS, aJm, AR, gZ, aS, y, iu, us); #c1(CDKN2C) c2(NP_001253) c3(cyclin-dependent kinase 4 inhibitor C) c4( ) c5( ) c6( ) c7(T, pY, al, eX, fd, hy, v, hF, Fc, eA, ip, bv, Do, aT, h, eV, K, jf, yo, d, BN, s, B, aW, Y, t, cm, bw, ck, iO, gk, amS, YE, Dy, eM, nB, Vy, iS, hR, OD, be, y, vt, ne); #c1 (CDKN2D) c2(NP_001791) c3(cyclin-dependent kinase 4 inhibitor D) c4(121147) c5(121148, 121149, 121150, 121151, 121152, 121153, 121154, 121155, 121156, 121157) c6( ) c7(T, aC, pY, aAx, aA, Hn, ci, hF, V, eA, mj, kH, aT, io, K, hH, pq, eL, Lg, jf, je, aW, aoo, cr, lo, eF, t, bw, bz, aJx, iM, bP, c, Jr, eD, AR, U, m, y, qR); #c1(CDKN3) c2(NP_001124323) c3(cyclin-dependent kinase inhibitor 3 isoform 2) c4(48682) c5(48683, 48684, 48685, 48686, 48687, 48688) c6( ) c7(bM, T, ea, if, fd, ci, xE, aE, aEX, M, K, hu, eL, Qe, yo, dH, aW, uY, g, as, eF, cm, axp, bw, eM, cj, iS, azg, m, k, dD, ht, aj, qN, ai, eW, aDF, co, pe, kH, h, dl, ey, aq, dw, iO, KT, bP, hk, eD, hR, rx, it, B, bL, jx, aA, eA, J, er, cF, Vr, ahL, aT, j, ae, jf, s, nO, oV, Y, en, apx, t, iK, ck, nM, Dy, iM, im, bt, hS, id, pY, ml, jh, Fb, eq, hF, iJ, ad, hX, dP, d, je, ff, eH, aF, iR, bF, aS, bC, y, kS); #c1(CDKN3) c2(NP_005183) c3(cyclin-dependent kinase inhibitor 3 isoform 1) c4(153695, 153696, 153697, 153698) c5(153699, 153700, 153701, 153702, 153703, 153704, 153705, 153706, 153707, 153708, 153709, 153710, 153711, 153712, 153713, 153714, 153715, 153716) c6( ) c7(bM, T, ea, if, fd, ci, xE, aE, aEX, M, K, hu, eL, de, yo, dH, aW, uY, g, as, eF, cm, axp, bw, eM, cj, iS, azg, m, k, dD, ht, aj, qN, ai, eW, aDF, co, pe, kH, h, dL, ey, aq, dw, iO, kT, bP, hk, eD, hR, rx, it, B, bL, jx, aA, eA, J, er, cf, Vr, ahl, aT, j, ae, jf, s, nO, oV, Y, en, apx, t, iK, ck, nM, Dy, iM, im, bt, hS, id, pY, ml, jh, Fb, eq, hF, iJ, ad, hX, dP, d, je, ff, eH, aF, iR, bF, aS, bC, y, kS); #c1(CDKN3) c2(XP_011534655) c3(cyclin-dependent kinase inhibitor 3 isoform X1) c4( ) c5(331160, 331161, 331162, 331163, 331164, 331165) c6( ) c7(bM, T, ea, if, fd, ci, xE, aE, aEX, M, K, hu, eL, Qe, yo, dH, aW, uY, g, as, eF, cm, axp, bw, eM, cj, iS, azg, m, k, dD, ht, aj, qN, ai, eW, aDF, co, pe, kH, h, dL, ey, aq, dw, iO, kT, bP, hk, eD, hR, rx, it, B, bL, jx, aA, eA, J, er, cF, Vr, ahl, aT, j, ae, jf, s, nO, oV, Y, en, apx, t, iK, ck, nM, Dy, iM, im, bt, hS, id, pY, ml, jh, Fb, eq, hF, iJ, ad, hX, dP, d, je, ff, eH, aF, iR, bF, aS, bC, y, kS); #c1(CDNF) c2(NP_001025125) c3(cerebral dopamine neurotrophic factor precursor) c4(29347, 29348) c5(29349, 29350, 29351, 29352, 29353, 29354, 29355, 29356, 29357, 29358, 29359, 29360, 29361, 29362, 29363) c6(29364, 29365, 29366) c7(an); #c1(CD01) c2(NP_001792) c3(cysteine dioxygenase type 1) c4(121158) c5(121159, 121160, 121161, 121162, 121163, 121164) c6( ) c7(bL, T, jz, d, eU, xR, aW, hl, gX, aT, fQ, r, cz, BV, q, MG); #c1(CDON) c2(XP_011541167) c3(cell adhesion molecule-related/down-regulated by oncogenes isoform X2) c4(339748) c5(339749, 339750, 339751, 339752, 339753, 339754) c6( ) c7(BV, ZW, MG, aJZ); #c1(CDON) c2(XP_011541168) c3(cell adhesion molecule-related/down-regulated by oncogenes isoform X1) c4(339755) c5(339756, 339757, 339758, 339759, 339760, 339761) c6( ) c7(BV, ZW, MG, aJZ); #c1(CDR1) c2(NP_004056) c3(cerebellar degeneration-related antigen 1) c4(143192) c5(143193, 143194, 143195, 143196, 143197, 143198) c6( ) c7(oO, T, aw, fd, mC, br, sO, aT, h, MG, aW, eZ, lo, PG, pm, bw, ac, pc, cj, rH, fl, bt, L); #c1(CDR2) c2(NP_001793) c3(cerebellar degeneration-related protein 2) c4(121165) c5(121166, 121167, 121168, 121169, 121170, 121171) c6( ) c7(Qi, T, akJ, aT, cj, asa, By, h, m, t, V, it, MG); #c1(CDS2) c2(NP_003809) c3(phosphatidate cytidylyltransferase 2) c4(141051, 141052, 141053) c5(141054, 141055, 141056, 141057, 141058, 141059) c6( ) c7(cd, oQ); #c1(CDS2) c2(XP_006723723) c3(phosphatidate cytidylyltransferase 2 isoform X1) c4(295319) c5(295320, 295321, 295322, 295323, 295324, 295325) c6( ) c7(cd, oQ); #c1(CDS2) c2(XP_011527692) c3(phosphatidate cytidylyltransferase 2 isoform X2) c4( ) c5(322617, 322618, 322619, 322620, 322621, 322622, 322623, 322624, 322625, 322626, 322627, 322628) c6( ) c7(cd, o); #c1(CDT1) c2(NP_112190) c3(DNA replication factor Cdt1) c4(220717) c5(220718, 220719, 220720, 220721, 220722, 220723) c6( ) c7(T, fM, dE, aT, aKa, ali, m, k, t); #c1(CDV3) c2(NP_001127894) c3(protein CDV3 homolog isoform a) c4(49433) c5(49434, 49435, 49436, 49437, 49438, 49439) c6( ) c7(q, r); #c1(CDV3) c2(NP_001127895) c3 (protein CDV3 homolog isoform c) c4( ) c5(49440, 49441, 49442, 49443, 49444) c6( ) c7(q, r); #c1(CDV3) c2(NP_060018) c3(protein CDV3 homolog isoform b) c4(193682) c5(193683, 193684, 193685, 193686, 193687, 193688) c6( ) c7(q, r); #c1(CDV3) c2(XP_011511263) c3(protein CDV3 homolog isoform X4) c4(301768) c5(30/769) c6( ) c7(q, r); #c1(CDX1) c2(NP_001795) c3(homeobox protein CDX-1) c4(121172, 121173, 121174) c5(121175, 121176, 121177, 121178, 121179, 121180, 121181, 121182, 121183, 121184, 121185, 12186, 121187, 121188, 121189, 121190, 121191, 121192, 121193) c6( ) c7(T, jW, aD, aA, bB, ci, q, dP, aE, Tj, pl, aKb, s, ev, az, r, kt, aF, g, bP, bE, gX, apy, bt, cE, X); #c1(CDX2) c2(NP_001256) c3(homeobox protein CDX-2) c4( ) c5( ) c6( ) c7(Fv, T, aC, pf, atj, bB, ci, x, aE, gH, pX, auB, hu, cp, Qe, JI, aW, az, LZ, kt, NR, g, eM, aep, m, k, cE, ht, NT, n, ft, jz, Yc, gd, hB, pl, AQ, fe, aq, kp, hL, at, Te, ZZ, B, yk, aA, AD, V, q, akc, eJ, Yn, aT, ae, bA, s, NE, iU, Y, ev, r, akY, en, cl, t, ik, ake, ck, sS, aJt, gX, bt, aD, oH, hF, ad, akd, av, abk, l, NS, io, BO, qt, ff, aF, bz, bF, bE, X, MC, iu); #c1(CDX4) c2(NP_005184) c3(homeobox protein CDX-4) c4(153717) c5(153718, 153719, 153720, 153721, 153722, 153723, 153724) c6( ) c7(ck, hF, bt, gX); #c1(CDY1B) c2(NP_001003894) c3(testis-specific chromodomain protein Y 1 isoform a) c4(20250) c5(20251, 20252, 20253, 20254, 20255, 20256, 20257, 20258, 20259, 20260) c6( ) c7(vH, UI, ND, fd); #c1(CDY1B) c2(NP_001003895) c3(testis-specific chromodomain protein Y 1 isoform b) c4(20261, 20262) c5(20263, 20264, 20265, 20266, 20267, 20268, 20269, 20270, 20271, 20272, 20273, 20274, 20275, 20276, 20277, 20278, 20279, 20280, 20281) c6( ) c7(vH, UI, ND, fd); #c1(CDY2B) c2(NP_001001722) c3(testis-specific chromodomain protein Y 2) c4(18996) c5(18997, 18998, 18999, 19000, 19001, 19002, 19003, 19004, 19005, 19006) c6( ) c7(vH, pt, UI); #c1(CDYL) c2(NP_001137442) c3(chromodomain Y-like protein isoform d) c4( ) c5(55750, 55751, 55752, 55753, 55754, 55755, 55756, 55757) c6( ) c7(at, ft); #c1(CDYL) c2(NP_004815) c3(chromodomain Y-like protein isoform a) c4(150009, 150010, 150011) c5(150012, 150013, 150014, 150015, 150016, 150017, 150018, 150019, 150020, 150021, 150022, 150023, 150024, 150025) c6( ) c7(at, ft); #c1(CDYL2) c2(NP_689555) c3(chromodomain Y-like protein 2) c4(243097) c5(243098, 243099, 243100, 243101, 243102, 243103) c6( ) c7(dy, ac); #c1(CEACAM1) c2(NP_001020083) c3(carcinoembryonic antigen-related cell adhesion molecule 1 isoform 2 precursor) c4(28111) c5(28112, 28113, 28114, 28115, 28116, 28117, 28118, 28119, 28120, 28121, 28122, 28123, 28124, 28125, 28126, 28127, 28128, 28129, 28130, 28131, 28132, 28133, 28134) c6( ) c7(oO, bL, T, fR, wu, aA, bB, v, fr, ci, q, ad, eJ, av, Bl, hd, amB, aE, Vr, M, aT, dl, wm, ae, eL, bA, B, s, aW, Y, r, aq, pD, g, cl, ek, ac, eM, nB, bE, AM, hL, wR, X); #c1(CEACAM1) c2(NP_001171742) c3(carcinoembryonic antigen-related cell adhesion molecule 1 isoform 4 precursor) c4(71470) c5(71471, 71472, 71473, 71474, 71475, 71476) c6( ) c7(oO, bL, T, FR, wu, aA, bB, v, fr, ci, q, ad, eJ, av, Bl, hd, amB, aE, Vr, M, aT, dl, wm, ae, eL, bA, B, s, aW, Y, r, aq, pD, g, cl, ek, ac, eM, nB, bE, AM, hL, wR, X); #c1(CEACAM1) c2(NP_001171744) c3(carcinoembryonic antigen-related cell adhesion molecule I isoform 3 precursor) c4(71477, 71478, 71479) c5(71480, 71481, 71482, 71483, 71484, 71485) c6( ) c7(oO, bL, T, FR, wu, aA, bB, v, fr, ci, q, ad, eJ, av, Bl, hd, amB, aE, Vr, M, aT, dl, wm, ae, eL, bA, B, s, aW, Y, r, aq, pD, g, cl, ek, ac, eM, nB, bE, AM, hL, wR, X); #c1(CEACAM1) c2(NP_001171745) c3(carcinoembryonic antigen-related cell adhesion molecule 1 isoform 5 precursor) c4(71486) c5(71487, 71488, 71489, 71490, 71491, 71492, 71493, 71494, 71495, 71496, 71497, 71498) c6( ) c7(oO, bL, T, fR, wu, aA, bB, v, fr, ci, q, ad, eJ, av, Bl, hd, amB, aE, Vr, M, aT, dL, wm, ae, eL, bA, B, s, aW, Y, r, aq, pD, g, cl, ek, ac, eM, nB, bE, AM, hL, wR, X); #c1(CEACAM1) c2(NP_001703) c3(carcinoembryonic antigen-related cell adhesion molecule 1 isoform 1 precursor) c4(120241, 120242, 120243, 120244, 120245) c5(120246, 120247, 120248, 120249, 120250, 120251, (20252, 120253, 120254) c6( ) c7(oO, bL, T, fR, wu, aA, bB, v, fr, ci, q, ad, eJ, av, Bl, hd, amB, aE, Vr, M, aT, dL, wm, ae, eL, bA, B, s, aW, Y, r, aq, pD, g, cl, ek, ac, eM, nB, bE, AM, hL, wR, X); #c1(CEACAM1) c2(XP_011525508) c3(carcinoembryonic antigen-related cell adhesion molecule 1 isoform X1) c4(319909) c5(319910, 319911, 319912, 319913, 319914, 319915, 319916, 319917, 319918, 319919, 319920, 319921, 319922, 319923, 319924, 319925, 319926, 319927, 319928) c6( ) c7(oO, bL, T, fR, wu, aA, bB, v, fr, ci, q, ad, eJ, av, Bl, hd, amB, aE, Vr, M, aT, dl, wm, ae, eL, bA, B, s, aW, Y, r, aq, pD, g, cl, ek, ac, eM, nB, bE, AM, hL, wR, X); #c1(CEACAM16) c2(NP_001034302) c3(carcinoembryonic antigen-related cell adhesion molecule 16 precursor) c4(31650, 31651) c5(31652, 31653, 31654, 31655, 31656, 31657, 31658, 31659, 31660, 31661, 31662, 31663) c6( ) c7(akf, akg, fM); #c1(CEACAM19) c2(NP_001121365) c3(carcinoembryonic antigen-related cell adhesion molecule 19 isoform 1 precursor) c4(45777) c5(45778, 45779, 45780, 45781, 45782, 45783) c6( ) c7(aW, aT); #c1(CEACAM19) c2(NP_064604) c3(carcinoembryonic antigen-related cell adhesion molecule 19 isoform 2 precursor) c4(203251) c5(203252, 203253, 203254, 203255, 203256, 203257) c6( ) c7(aW, aT); #c1(CEACAM21) c2(XP_006725457) c3(carcinoembryonic antigen-related cell adhesion molecule 21 isoform X1) c4(296808) c5(296809, 296810, 296811, 296812, 296813, 296814, 296815) c6( ) c7(ao); #c1(CEACAM21) c2(XP_006725458) c3(carcinoembryonic antigen-related cell adhesion molecule 21 isoform X2) c4(296816) c5(296817, 296818, 296819, 296820, 296821, 296822) c6( ) c7(ao); #c1(CEACAM3) c2(NP_001264092) c3(carcinoembryonic antigen-related cell adhesion molecule 3 isoform 2 precursor) c4(97513) c5(97514, 97515, 97516, 97517, 97518, 97519, 97520, 97521, 97522, 97523, 97524, 97525, 97526, 97527, 97528) c6( ) c7(T, dA, if, NT, aA, bB, n, ci, ze, qN, q, eW, x, Vp, bv, av, ib, aE, iu, jz, nv, Qg, gd, aT, cU, dL, ox, Tj, eL, de, B, BO, s, JI, aW, ff, r, af, CQ, en, YF, cl, t, aoG, bz, g, cm, ck, bP, eM, bE, dc, gX, m, X, ao); #c1(CEACAM3) c2(NP_001806) c3(carcinoembryonic antigen-related cell adhesion molecule 3 isoform 1 precursor) c4(121279) c5(121280, 121281, 121282, 121283, 121284, 121285) c6( ) c7(T, dA, if, NT, aA, bB, n, ci, ze, qN, q, eW, x, Vp, bv, av, ib, aE, iu, jz, nv, Qg, gd, aT, cU, dL, ox, Tj, eL, de, B, BO, s, JI, aW, ff, r, af, CO, en, YF, cl, t, aoG, bz, g, cm, ck, bP, eM, bE, dc, gX, m, X, ao); #c1(CEACAM4) c2(NP_001808) c3(carcinoembryonic antigen-related cell adhesion molecule 4 precursor) c4(121297) c5(121298, 121299, 121300, 121301, 121302, 121303) c6( ) c7(x, T, eJ, s, iU, bE, r, bB, ji, ci, K, g, ho, q); #c1(CEACAM5) c2(NP_001278413) c3(carcinoembryonic antigen-related cell adhesion molecule 5 preproprotein) c4(109702) c5(109703, 109704, 109705, 109706, 109707, 109708, 109709, 109710, 109711, 109712) c6( ) c7(T, aC, if, bB, ci, x, Vp, bv, aE, K, hu, eL, Qe, JI, aW, az, g, cm, qJ, eM, nB, AM, dc, m, iu, aki, oO, ht, NT, n, qN, eW, b, ft, jz, zw, Xe, gd, cU, ic, dL, hB, aDW, dx, pl, Yl, ia, aq, CO, YF, kk, cM, ar, iO, bP, Pl, pg, it, B, fM, dA, aA, ze, q, eA, Yn, nv, Qg, aT, cy, Tj, ae, s, NE, iU, oV, Y, ev, r, lo, en, cl, aoG, t, ck, pa, gX, nZ, Dd, ad, hX, av, ib, NS, ji, d, BO, afe, ff, akh, aF, iV, bz, bF, bE, i, X); #c1(CEACAM6) c2(NP_002474) c3(carcinoembryonic antigen-related cell adhesion molecule 6 preproprotein) c4(129143, 129144) c5(129145, 129146, 129147, 129148, 129149, 129150) c6( ) c7(oO, Mb, T, aC, aA, bB, n, fd, ci, q, ad, eJ, x, iu, jz, aE, aT, ji, K, ox, ae, aa, s, aW, Y, r, fe, aF, pD, g, iV, ho, t, iK, ck, eM, bE, pg, k, X, ao); #c1(CEACAM6) c2(XP_011525292) c3(carcinoembryonic antigen-related cell adhesion molecule 6 isoform X1) c4( ) c5(319758) c6( ) c7(oO, Mb, T, aC, aA, bB, n, fd, ci, q, ad, eJ, x, iu, jz, aE, aT, ji, K, ox, ae, aa, s, aW, Y, r, fe, aF, pD, g, iV, ho, t, ik, ck, eM, bE, pg, k, X, ao); #c1(CEACAM7) c2(NP_001278414) c3(carcinoembryonic antigen-related cell adhesion molecule 7 preproprotein) c4(109713, 109714, 109715) c5(109716, 109717, 109718, 109719, 109720, 109721, 109722, 109723, 109724, 109725, 109726, 109727, 109728, 109729, 109730, 109731, 109732, 109733, 109734, 109735, 109736, 109737, 109738, 109739) c6(109740, 109741) c7(T, dA, if, NT, aA, bB, n, ci, ze, qN, q, eW, x, Vp, bv, av, ib, aE, iu, nv, Qg, gd, aT, c, dL, Tj, eL, Qe, B, BO, s, JI, aW, ev, ff, r, aF, CQ, en, YF, cl, t, aoG, bz, g, cm, bP, eM, bE, dc, gX, m, X, BA); #c1(CEACAM8) c2(NP_001807) c3(carcinoembryonic antigen-related cell adhesion molecule & precursor) c4(121286) c5(121287, 121288, 121289, 121290, 121291, 121292, 121293, 121294) c6(121295, 121296) c7(x, akc, bw, n, t, fh); #c1(CEBPA) c2(NP_001274353) c3(CCAAT/enhancer-binding protein alpha isoform c) c4( ) c5(106237, 106238) c6( ) c7(bL, T, Ve, fM, xu, aC, id, yk, e, iW, jh, fd, aj, hy, j, v, hF, br, ad, av, aT, dz, hn, K, jj, dQ, ae, af, iU, hG, et, aW, ff, eH, fc, aq, gm, yq, en, akk, akj, cM, bz, eF, cm, ik, iR, ck, iO, bw, iT, hk, bo, k, cP, ei, bt, B); #c1(CEBPA) c2(NP_004355) c3(CCAAT/enhancer-binding protein alpha isoform a) c4(145618) c5(145619, 145620, 145621, 145622, 145623) c6( ) c7(bL, T, Ve, fM, xu, aC, id, yk, e, iW, jh, fd, aj, hy, j, v, hF, br, ad, av, aT, dz, hn, K, jj, de, ae, af, iU, hG, et, aW, ff, eH, fc, aq, gm, yq, en, akk, akj, cM, bz, eF, cm, ik, iR, ck, iO, bw, iT, hk, bo, k, cP, ei, bt, B); #c1(CEBPB) c2(NP_005185) c3(CCAAT/enhancer-binding protein beta isoform a) c4(153725) c5(153726, 153727, 153728, 153729, 153730, 153731) c6( ) c7(bL, T, dA, fR, fG, e, zt, jh, bB, aj, v, ci, C, q, aR, J, ad, akl, ai, eA, av, eW, aE, azq, aT, h, bl, K, ji, jj, ae, eu, d, s, aW, ff, fc, eZ, r, akB, aq, lo, en, dw, iR, bz, op, ck, iD, cT, nM, pc, GU, c, nB, bE, eD, vA, AH, cj, uo, zu, bt, KS, B, y); #c1(CEBPD) c2(NP_005186) c3(CCAAT/enhancer-binding protein delta) c4(153732, 153733) c5(153734, 153735, 153736, 153737, 153738, 153739, 153740, 153741, 153742, 153743, 153744, 153745, 153746) c6( ) c7(T, id, if, cz, v, j, ci, C, qN, q, aR, ad, Kx, XX, cc, aT, h, bl, ae, af, qw, d, aW, ff, r, dq, en, dR, ac, bP, hk, U, Kj, ei, y, bt, B); #c1(CEBPE) c2(NP_001796) c3(CCAAT/enhancer-binding protein epsilon) c4(121194) c5(121195, 121196, 121197, 121198, 121199, 121200, 121201) c6( ) c7(ck, x, aKm, hn, k, jj, bw, bt); #c1(CEBPG) c2(NP_001239225) c3(CCAAT/enhancer-binding protein gamma) c4(88383) c5(88384, 88385, 88386, 88387, 88388, 88389, 88390) c6( ) c7(av, T, co, ci, bt); #c1(CEBPZ) c2(NP_005751) c3(CCAAT/enhancer-binding protein zeta) c4(158996, 158997) c5(158998, 158999, 159000, 159001, 159002, 159003, 159004, 159005, 159006, 159007, 159008, 159009, 159010) c6( ) c7(ck, T, fX, cm, bS, lR, ct, Z, hF, bt, pb, aBl, yR); #c1(CECR1) c2(NP_803124) c3(adenosine deaminase CECR1 isoform b) c4(252491) c5(252492, 252493, 252494, 252495, 252496, 252497, 252498, 252499) c6(252500, 252501) c7(Cx, gV, aC, eG, dA, ac, hG, aKn, dV, kp, j, y, eA, hv); #c1(CECR1) c2(XP_011544435) c3(adenosine deaminase CECR1 isoform X1) c4(343541, 343542, 343543, 343544) c5(343545, 343546, 343547, 343548, 343549, 343550, 343551, 343552, 343553, 343554, 343555, 343556, 343557, 343558, 343559, 343560, 343561, 343562, 343563, 343564, 343565, 343566, 343567, 343568, 343569, 343570, 343571, 343572, 343573, 343574, 343575, 343576, 343577, 343578, 343579, 343580, 343581, 343582, 343583, 343584, 343585, 343586, 343587, 343588, 343589, 343590, 343591) c6(343592, 343593, 343594, 343595, 343596, 343597, 343598, 343599, 343600, 343601, 343602, 343603) c7(Cx, gV, aC, eG, dA, ac, hG, aKn, dV, kp, j, y, eA, hv); #c1(CECR2) c2(NP_001276976) c3(cat eye syndrome critical region protein 2 isoform 1) c4( ) c5(108883, 108884, 108885, 108886, 108887, 108888) c6( ) c7(anN, ako, RX, agd); #c1(CECR2) c2(XP_006724141) c3(cat eye syndrome critical region protein 2 isoform X3) c4(295817) c5( ) c6( ) c7(anN, ako, RX, agd); #c1(CECR2) c2(XP_006724142) c3(cat eye syndrome critical region protein 2 isoform X7) c4( ) c5(295818, 295819, 295820, 295821, 295822, 295823) c6( ) c7(anN, ako, RX, agd); #c1(CECR2) c2(XP_011544430) c3(cat eye syndrome critical region protein 2 isoform X1) c4(343534) c5(343535, 343536, 343537, 343538, 343539, 343540) c6( ) c7(anN, ako, RX, agd); #c1(CEL) c2(NP_001798) c3(bile salt-activated lipase precursor) c4(121202, 121203, 121204) c5(121205, 121206, 121207, 121208, 121209, 121210, 121211, 121212, 121213, 121214, 121215, 121216, 121217, 121218, 121219, 121220, 121221, 121222) c6( ) c7(oO, bM, T, aC, ea, aku, e, bB, aKs, ci, Jt, akq, bW, dV, K, zy, H, aKy, YC, g, Im, dc, oy, cj, m, R, aKr, dD, aw, NT, aj, akt, alZ, akz, akx, h, mJ, ks, aq, pm, aKv, ej, Yl, ar, aKA, bP, at, Br, B, fM, fG, asB, cR, v, j, alx, J, q, bq, s, aKD, r, en, t, pb, ig, akp, dT, fD, aKB, ob, df, akw, jh, eq, fu, iG, hF, hX, aHF, vB, MG, qE, uQ, BO, ff, Kf, anW, dr, LB, bF, bE, dO, uR, afd, akC, aEN, ei); #c1(CELA1) c2(NP_001962) c3(chymotrypsin-like elastase family member 1) c4(122662) c5(122663, 122664, 122665, 122666, 122667, 122668, 122669, 122670) c6(122671, 122672) c7(al, ao, OC, wC); #c1(CELA3B) c2(XP_011539434) c3(chymotrypsin-like elastase family member 3B isoform X1) c4(337586) c5( ) c6( ) c7(ar, akF, aKE, aKG, hJ, ao); #c1(CELA3B) c2(XP_011539435) c3(chymotrypsin-like elastase family member 38 isoform X2) c4(337587) c5(337588, 337589, 337590, 337591, 337592, 337593, 337594, 337595) c6(337596, 337597) c7(ar, aKF, aKE, akG, hJ, ao); #c1(CELF1) c2(NP_001166110) c3(CUGBP Elav-like family member 1 isoform 4) c4(68173) c5(68174, 68175, 68176, 68177, 68178, 68179, 68180, 68181, 68182, 68183, 68184, 68185, 68186, 68187, 68188, 68189, 68190, 68191, 68192, 68193) c6( ) c7(wK, h, aq, v, bl, iR, dR); #c1(CELF1) c2(NP_001166111) c3(CUGBP Elav-like family member 1 isoform 5) c4(68194, 68195) c5(68196, 68197, 68198, 68199, 68200) c6( ) c7(wK, h, aq, v, bl, iR, dR); #c1(CELF1) c2(NP_006551) c3(CUGBP Elav-like family member 1 isoform 1) c4(166170) c5(166171, 166172, 166173, 166174, 166175, 166176, 166177, 166178, 166179, 166180, 166181) c6( ) c7(wK, h, aq, v, bl, iR, dR); #c1(CELF1) c2(XP_011518153) c3(CUGBP Elav-like family member 1 isoform X1) c4(312179) c5(312180, 312181, 312182, 312183, 312184, 312185) c6( ) c7(wK, h, aq, v, bl, iR, dR); #c1(CELF1) c2(XP_011518154) c3(CUGBP Elav-like family member 1 isoform X4) c4(312186) c5(312187, 312188, 312189, 312190, 312191) c6( ) c7(wK, h, aq, v, bl, iR, dR); #c1(CELF1) c2(XP_011518155) c3(CUGBP Elav-like family member 1 isoform X5) c4(312192) c5( ) c6( ) c7(wK, h, aq, v, bl, iR, dR); #c1(CELF1) c2(XP_011518161) c3 (CUGBP Elav-like family member 1 isoform X2) c4(312193, 312194) c5(312195, 312196, 312197, 312198, 312199, 312200) c6( ) c7(wK, h, aq, v, bl, iR, dR); #c1(CELF2) c2(NP_001020247) c3(CUGBP Elav-like family member 2 isoform 1) c4( ) c5(28352, 28353, 28354, 28355, 28356, 28357, 28358, 28359, 28360, 28361, 28362, 28363, 28364, 28365) c6( ) c7(D, cF, aE, wK, aW, RX, aT, kf, akl, cd, j, ey, aKH, X, dR); #c1(CELF2) c2(NP_001077060) c3(CUGBP Elav-like family member 2 isoform 4) c4(37629) c5(37630, 37631, 37632, 37633, 37634, 37635, 37636, 37637, 37638, 37639) c6( ) c7(D, cF, aE, wK, aW, RX, aT, kf, akl, cd, j, ey, aKH, X, dR); #c1(CELF2) c2(NP_006552) c3(CUGBP Elav-like family member 2 isoform 2) c4(166182) c5(166183, 166184, 166185, 166186, 166187, 166188, 166189, 166190, 166191, 166192, 166193) c6( ) c7(D, cF, aE, wK, aW, RX, aT, kf, akl, cd, j, ey, aKH, X, dR); #c1(CELF2) c2(XP_005252411) c3(CUGBP Elav-like family member 2 isoform X4) c4(271721) c5(271722, 271723, 271724) c6( ) c7(D, cF, aE, wK, aW, RX, aT, kf, akl, cd, j, ey, aKH, X, dR); #c1(CELF2) c2(XP_006717432) c3(CUGBP Elav-like family member 2 isoform X2) c4( ) c5(291083) c6( ) c7(D, cF, aE, wK, aW, RX, aT, kf, akl, cd, j, ey, aKH, X, dR); #c1(CELF2) c2(XP_0067 (7433) c3(CUGBP Elav-like family member 2 isoform X3) c4(291084) c5(291085) c6( ) c7(D, cF, aE, wK, aW, RX, aT, kf, akl, cd, j, ey, aKH, X, dR); #c1(CELF2) c2(XP_006717436) c3(CUGBP Elav-like family member 2 isoform X10) c4(291086) c5(291087, 291088, 291089, 291090, 291091, 291092) c6( ) c7(D, cF, aE, wK, aW, RX, aT, kf, akl, cd, j, ey, aKH, X, dR); #c1(CELF2) c2(XP_011517596) c3(CUGBP Elav-like family member 2 isoform X1) c4(311355, 311356) c5(311357, 311358, 311359, 311360, 311361, 311362, 311363, 311364, 311365, 311366) c6( ) c7(D, cF, aE, wK, aW, RX, aT, kf, akl, cd, j, ey, aKH, X, dR); #c1(CELF2) c2(XP_011517599) c3(CUGBP Elav-like family member 2 isoform X7) c4( ) c5(311367, 311368, 311369, 311370, 311371, 311372) c6( ) c7(D, cF, aE, wK, aW, RX, aT, kf, akl, cd, j, ey, aKH, X, dR); #c1(CELF4) c2(NP_001020258) c3(CUGBP Elav-like family member 4 isoform 2) c4(28373) c5(28374, 28375, 28376, 28377, 28378, 28379) c6( ) c7(bL, aw, ha, r, v, Fl, aR); #c1(CELF4) c2(NP_001020259) c3(CUGBP Elav-like family member 4 isoform 3) c4(28380) c5(28381, 28382, 28383, 28384, 28385, 28386) c6( ) c7(bL, aw, ha, r, v, Fl, aR); #c1(CELF4) c2(NP_001020260) c3(CUGBP Elav-like family member 4 isoform 4) c4(28387) c5(28388, 28389, 28390, 28391, 28392, 28393) c6( ) c7(bL, aw, ha, r, v, Fl, aR); #c1(CELF4) c2(NP_064565) c3(CUGBP Elav-like family member 4 isoform 1) c4(202997) c5(202998, 202999, 203000, 203001, 203002, 203003) c6( ) c7(bL, aw, ha, r, v, Fl, aR); #c1(CELF4) c2(XP_011524388) c3(CUGBP Elav-like family member 4 isoform X7) c4( ) c5(319018) c6( ) c7(bL, aw, ha, r, v, Fl, aR); #c1(CELF4) c2(XP_011524394) c3(CUGBP Elav-like family member 4 isoform X14) c4(319019) c5( ) c6( ) c7(bL, aw, ha, r, v, Fl, aR); #c1(CELF4) c2(XP_011524398) c3(CUGBP Elav-like family member 4 isoform X18) c4( ) c5(319020, 319021, 319022, 319023, 319024) c6( ) c7(bL, aw, ha, r, v, Fl, aR); #c1(CELF5) c2(NP_001166144) c3(CUGBP Elav-like family member 5 isoform 2) c4(68324, 68325) c5(68326, 68327, 68328, 68329, 68330, 68331, 68332, 68333, 68334, 68335, 68336, 68337, 68338) c6( ) c7(pN); #c1(CELF5) c2(XP_006722895) c3(CUGBP Elav-like family member 5 isoform X1) c4(294920) c5(294921, 294922, 294923, 294924, 294925, 294926) c6( ) c7(pN); #c1(CELF6) c2(NP_001166155) c3(CUGBP Elav-like family member 6 isoform 2) c4(68353, 68354) c5(68355, 68356, 68357, 68358, 68359, 68360, 68361, 68362, 68363, 68364, 68365, 68366, 68367, 68368, 68369, 68370) c6( ) c7(bN); #c1(CELF6) c2(NP_001166156) c3(CUGBP Elav-like family member 6 isoform 3) c4(68371) c5(68372, 68373, 68374, 68375, 68376, 68377) c6( ) c7(bN); #c1(CELF6) c2(NP_443072) c3(CUGBP Elav-like family member 6 isoform 1) c4(229242, 229243, 229244) c5(229245, 229246, 229247, 229248, 229249, 229250, 229251, 229252, 229253, 229254, 229255, 229256, 229257, 229258, 229259, 229260, 229261, 229262) c6( ) c7(bN); #c1(CELSR1) c2(NP_055061) c3(cadherin EGF LAG seven-pass G-type receptor 1 precursor) c4(178668) c5(178669, 178670, 178671, 178672, 178673, 178674) c6( ) c7(lp, eu, ac, M); #c1(CELSR1) c2(XP_006724446) c3(cadherin EGF LAG seven-pass G-type receptor 1 isoform X1) c4(295982, 295983) c5( ) c6( ) c7(lp, eu, ac, M); #c1(CELSR1) c2(XP_011528855) c3(cadherin EGF LAG seven-pass G-type receptor 1 isoform X2) c4(324228) c5(324229, 324230, 324231, 324232, 324233, 324234, 324235, 324236, 324237, 324238, 324239, 324240) c6( ) c7(lp, eu, ac, M); #c1(CELSR3) c2(NP_001398) c3(cadherin EGF LAG seven-pass G-type receptor 3 precursor) c4(117577, 117578) c5(117579, 117580, 117581, 117582, 117583, 117584, 117585, 117586, 117587, 117588, 117589, 117590, 117591, 117592) c6( ) c7(li, c, lm, pk, ae, ad); #c1(CEMIP) c2(NP_001280227) c3(cell migration-inducing and hyaluronan-binding protein precursor) c4(111496, 111497) c5(111498, 111499, 111500, 111501, 111502, 111503, 111504, 111505, 111506, 111507, 111508, 111509, 111510, 111511, 111512, 111513, 111514, 111515, 111516, 111517) c6( ) c7(T, s, bE, aW, aw, aT, bB, n, fr, g, Bh); #c1(CEMP1) c2(NP_001041677) c3(cementoblastoma-derived protein 1) c4(34279) c5(34280, 34281, 34282, 34283, 34284, 34285, 34286, 34287, 34288, 34289) c6( ) c7(eJ); #c1(CEND1) c2(NP_057648) c3(cell cycle exit and neuronal differentiation protein 1) c4(192048) c5(192049, 192050, 192051, 192052, 192053, 192054, 192055) c6( ) c7(cm, dr, ml); #c1(CENPA) c2(NP_001035891) c3(histone H3-like centromeric protein A isoform b) c4(33339) c5(33340, 33341, 33342, 33343, 33344, 33345) c6( ) c7(iu, T, B, kP, aW, r, aT, ey, m, g, pb, lR, q); #c1(CENPA) c2(NP_001800) c3(histone H3-like centromeric protein A isoform a) c4(121223) c5(121224, 121225, 121226, 121227, 121228, 121229) c6( ) c7(iu, T, B, kP, aW, r, aT, ey, m, g, pb, lR, q); #c1(CENPB) c2(NP_001801) c3(major centromere autoantigen B) c4(121230, 121231) c5(121232, 121233, 121234, 121235, 121236, 121237, 121238, 121239, 121240, 121241, (21242, (21243) c6( ) c7(uo, pb); #c1(CENPC) c2(NP_001803) c3(centromere protein C) c4(121244) c5(121245, 121246, 121247, 121248, (21249, 121250) c6( ) c7(pb, bK, jh, an); #c1(CENPE) c2(NP_001273663) c3(centromere-associated protein E isoform 2 proprotein) c4(105577) c5(105578, 105579, 105580, 105581, 105582, 105583) c6( ) c7(pb, T, ac, aT, cm, bu, QK, avx, B); #c1(CENPE) c2(NP_001804) c3(centromere-associated protein E isoform 1 proprotein) c4(121251, 121252) c5(121253, 121254, 121255, 121256, 121257, 121258, 121259, 121260, 121261, 121262, 121263, 121264) c6( ) c7(pb, T, ac, aT, cm, bu, QK, avx, B); #c1(CENPF) c2(NP_057427) c3(centromere protein F) c4(190752, 190753) c5(190754, 190755, 190756, 190757, 190758, 190759, 190760, 190761, 190762, 190763, 190764, 190765) c6( ) c7(T, ac, yw, aW, iB, ff, qK, afb, cj, aT, YP, ey, en, iK, ae, ad, YI); #c1(CENPH) c2(NP_075060) c3(centromere protein H) c4(213600, 213601) c5(213602, 213603, 213604, 213605, 213606, 213607, 213608, 213609, 213610, 213611, 213612, 213613) c6( ) c7(T, aA, r, aF, ci, nz, q); #c1(CENPJ) c2(NP_060921) c3(centromere protein J) c4(198693) c5(198694, 198695, 198696, 198697, 198698, 198699) c6( ) c7(ck, y, aii, hd, dA, aKK, pc, nM, AS, aKJ, WE, aT, cj, MC, ji, aon, QK, bt, avx, AN, aij); #c1(CENPJ) c2(XP_011533451) c3(centromere protein J isoform X1) c4(329567, 329568) c5(329569, 329570, 329571, 329572, 329573, 329574) c6( ) c7(ck, y, aii, hd, dA, aKK, pc, nM, AS, aKJ, WE, aT, cj, MC, ji, aon, QK, bt, avx, AN, aij); #c1 (CENPJ) c2(XP_011533452) c3(centromere protein J isoform X2) c4( ) c5(329575, 329576, 329577, 329578, 329579, 329580) c6( ) c7(ck, y, aii, hd, dA, aKK, pc, nM, AS, aKJ, WE, aT, cj, MC, ji, aon, QK, bt, avx, AN, aij); #c1 (CENPK) c2(NP_001253967) c3(centromere protein K) c4(94274, 94275) c5(94276, 94277, 94278, 94279, 94280, 94281, 94282, 94283, 94284, 94285, 94286, 94287, 94288, 94289, 94290, 94291, 94292, 94293, 94294, 94295) c6( ) c7(T, kP); #c1(CENPN) c2(NP_001094095) c3(centromere protein N isoform 1) c4(40642) c5(40643, 40644, 40645, 40646, 40647, 40648) c6( ) c7(ey); #c1(CENPN) c2(NP_001257402) c3(centromere protein N isoform 4) c4(95362) c5(95363, 95364, 95365, 95366, 95367, 95368) c6( ) c7(ey); #c1(CENPN) c2(NP_001257403) c3(centromere protein N isoform 5) c4(95369) c5(95370, 95371, 95372, 95373, 95374, 95375) c6( ) c7(ey); #c1(CENPN) c2(NP_060925) c3(centromere protein N isoform 3) c4(198707) c5(198708, 198709, 198710, 198711, 198712, 198713) c6( ) c7(ey); #c1(CENPN) c2(XP_006721299) c3(centromere protein N isoform X1) c4(293926) c5(293927, 293928, 293929, 293930, 293931, 293932) c6( ) c7(ey); #c1(CENPO) c2(NP_001186732) c3(centromere protein 0 isoform 2) c4(78442) c5(78443, 78444, 78445, 78446, 78447, 78448) c6( ) c7(bk); #c1(CENPO) c2(XP_006712165) c3(centromere protein 0 isoform X1) c4(286482) c5(286483, 286484, 286485, 286486, 286487, 286488, 286489, 286490, 286491, 286492) c6( ) c7(bK); #c1(CENPQ) c2(NP_060602) c3(centromere protein Q) c4(196797) c5(196798, 196799, 196800, 196801, 196802, 196803, 196804, 196805, 196806, 196807) c6( ) c7(j); #c1(CENPU) c2(NP_078905) c3(centromere protein U) c4(216018, 216019, 216020, 216021) c5(216022, 216023, 216024, 216025, 216026, 216027, 216028, 216029, 216030, 216031, 216032, 216033, 216034, 216035, 216036, 216037, 216038, 216039, 216040, 216041, 216042, 216043, 216044, 216045) c6( ) c7(If, d, eA); #c1(CENPV) c2(NP_859067) c3(centromere protein V) c4(255481) c5(255482, 255483, 255484, 255485, 255486, 255487) c6( ) c7(iM, je, au, Ne, By, EY, bC, zy, bt, hq); #c1(CENPW) c2(NP_001012525) c3 (centromere protein W isoform b) c4(25033) c5(25034, 25035, 25036, 25037, 25038, 25039) c6( ) c7(T, A, hb, bE, bB, xb, afu); #c1(CENPW) c2(NP_001273453) c3(centromere protein W isoform a) c4(104937) c5(104938, 104939, 104940, 104941, 104942, 104943) c6( ) c7(T, A, hb, bE, bB, xb, afu); #c1 (CENPW) c2(NP_001273454) c3(centromere protein W isoform c) c4(104944) c5(104945, 104946, 104947, 104948, 104949, 104950) c6( ) c7(T, A, hb, bE, bB, xb, afu); #c1(CEP112) c2(NP_001032402) c3(centrosomal protein of 112 kDa isoform b) c4(31303) c5(31304, 31305, 31306, 31307, 31308, 31309) c6( ) c7(V, aw); #c1(CEP112) c2(NP_001289820) c3(centrosomal protein of 112 kDa isoform c) c4(114673) c5(114674, 114675, 114676, 114677, 114678, 114679) c6( ) c7(V, aw); #c1(CEP112) c2(XP_011522763) c3(centrosomal protein of 112 kDa isoform X1) c4(317283) c5(317284, 317285, 317286, 317287, 317288, 317289, 317290, 317291, 317292, 317293) c6( ) c7(V, aw); #c1(CEP112) c2(XP_011522766) c3(centrosomal protein of 112 kDa isoform X6) c4(317294) c5(317295, 317296, 317297, 317298, 317299, 317300) c6( ) c7(V, aw); #c1(CEP120) c2(NP_001159698) c3(centrosomal protein of 120 kDa isoform 2) c4(64608) c5( ) c6( ) c7(e); #c1(CEP120) c2(NP_694955) c3(centrosomal protein of 120 kDa isoform 1) c4(245467, 245468) c5(245469, 245470, 245471, 245472, 245473, 245474, 245475, 245476, 245477, 245478, 245479, 245480, 245481, 245482, 245483, 245484) c6( ) c7(e); #c1(CEP120) c2(XP_005271958) c3(centrosomal protein of 120 kDa isoform X2) c4( ) c5(283647, 283648, 283649, 283650, 283651, 283652) c6( ) c7(e); #c1(CEP128) c2(XP_006720119) c3(centrosomal protein of 128 kDa isoform X1) c4(293251, 293252, 293253) c5(293254, 293255, 293256, 293257, 293258, 293259, 293260, 293261, 293262, 293263, 293264, 293265, 293266, 293267, 293268, 293269) c6( ) c7(j); #c1(CEP128) c2(XP_011534794) c3(centrosomal protein of 128 kDa isoform X2) c4( ) c5(331253, 331254, 331255, 331256, 331257) c6( ) c7(j); #c1(CEP128) c2(XP_011534797) c3(centrosomal protein of 128 kDa isoform X5) c4(331258, 331259, 331260, 331261) c5(331262) c6( ) c7(j); #c1(CEP(28) c2(XP_011534798) c3(centrosomal protein of 128 kDa isoform X6) c4( ) c5(331263, 331264, 331265, 331266, 331267, 331268, 331269, 331270, 331271, 331272, 331273, 331274, 331275, 331276, 331277, 331278, 331279, 331280, 331281, 331282, 331283, 331284, 331285, 331286, 331287) c6( ) c7(j); #c1(CEP131) c2(NP_001009811) c3(centrosomal protein of 131 kDa isoform b) c4(23687) c5(23688, 23689, 23690, 23691, 23692, 23693) c6( ) c7(gU); #c1(CEP131) c2(NP_055799) c3(centrosomal protein of 131 kDa isoform a) c4(183577) c5(183578, 183579, 183580, 183581, 183582, 183583) c6( ) c7(gU); #c1(CEP131) c2(XP_011522851) c3(centrosomal protein of 131 kDa isoform X3) c4(317334) c5(317335, 317336, 317337, 317338, 317339, 317340) c6( ) c7(gU); #c1(CEP135) c2(NP_079285) c3(centrosomal protein of 135 kDa) c4(217694, 217695) c5(217696, 217697, 217698, 217699, 217700, 217701) c6( ) c7(aKL, aii, aij); #c1(CEP135) c2(XP_006714118) c3(centrosomal protein of 135 kDa isoform X1) c4( ) c5(287574, 287575, 287576, 287577, 287578, 287579) c6( ) c7(aKL, aii, aij); #c1(CEP152) c2(NP_055800) c3(centrosomal protein of 152 kDa isoform 2) c4(183584) c5(183585, 183586, 183587, 183588, 183589, 183590) c6( ) c7(T, aii, WE, aKM, azZ, aKO, K, aKN, aij); #c1(CEP152) c2(XP_006720500) c3(centrosomal protein of 152 kDa isoform X1) c4(293527) c5(293528, 293529, 293530, 293531, 293532, 293533) c6( ) c7(T, aii, WE, aKM, azZ, ako, K, aKN, aij); #c1(CEP152) c2(XP_011519676) c3(centrosomal protein of 152 kDa isoform X3) c4(314339) c5( ) c6( ) c7(T, aii, WE, aKM, azZ, aKO, K, aKN, aij); #c1(CEP152) c2(XP_011519680) c3(centrosomal protein of 152 kDa isoform X7) c4(314340, 314341) c5(314342, 314343, 314344, 314345, 314346, 314347) c6( ) c7(T, aii, WE, aKM, azZ, aKO, K, aKN, aij); #c1(CEP152) c2(XP_011519681) c3(centrosomal protein of 152 kDa isoform X8) c4( ) c5(314348, 314349, 314350, 314351, 314352, 314353, 314354, 314355, 314356, 314357, 314358, 314359) c6( ) c7(T, aii, WE, aKM, azZ, aKO, K, aKN, aij); #c1(CEP162) c2(NP_055710) c3(centrosomal protein of 162 kDa isoform a) c4(182987) c5(182988, 182989, 182990, 182991, 182992, 182993) c6( ) c7(j); #c1(CEP164) c2(NP_001258862) c3(centrosomal protein of 164 kDa isoform 2) c4(96596, 96597) c5( ) c6( ) c7(akP, vm, ahD); #c1(CEP164) c2(NP_055771) c3(centrosomal protein of 164 kDa isoform 1) c4(183399) c5(183400, 183401, 183402, 183403, 183404, 183405) c6( ) c7(aKP, vm, ahD); #c1(CEP164) c2(XP_011540977) c3(centrosomal protein of 164 kDa isoform X6) c4(339505) c5( ) c6( ) c7(aKP, vm, ahD); #c1(CEP164) c2(XP_011540981) c3(centrosomal protein of 164 kDa isoform X11) c4(339506) c5(339507, 339508, 339509, 339510, 339511, 339512) c6( ) c7(akP, vm, ahD); #c1(CEP164) c2(XP_011540992) c3(centrosomal protein of 164 kDa isoform X20) c4( ) c5(339513, 339514, 339515, 339516, 339517, 339518, 339519, 339520, 339521, 339522, 339523, 339524, 339525, 339526, 339527, 339528, 339529, 339530, 339531, 339532, 339533, 339534) c6( ) c7(aKP, vm, ahD); #c1(CEP170) c2(NP_001035863) c3(centrosomal protein of 170 kDa isoform beta) c4(33300) c5(33301, 33302, 33303, 33304, 33305, 33306) c6( ) c7(V); #c1(CEP170) c2(NP_001035864) c3(centrosomal protein of 170 kDa isoform gamma) c4(33307) c5(33308, 33309, 33310, 33311, 33312, 33313, 33314, 33315, 33316, 33317, 33318, 33319, 33320) c6( ) c7(V); #c1(CEP170) c2(NP_055627) c3(centrosomal protein of 170 kDa isoform alpha) c4(182346) c5(182347, 182348, 182349, 182350, 182351, 182352, 182353, 182354, 182355, 182356, 182357, 182358) c6( ) c7(V); #c1(CEP170) c2(XP_011544742) c3(centrosomal protein of 170 kDa isoform X1) c4( ) c5(343815, 343816, 343817, 343818, 343819, 343820, 343821, 343822, 343823, 343824, 343825, 343826, 343827, 343828, 343829, 343830, 343831) c6( ) c7(V); #c1(CEP170) c2(XP_011544745) c3(centrosomal protein of 170 kDa isoform X4) c4(343832) c5( ) c6( ) c7(V); #c1(CEP(70) c2(XP_011544746) c3(centrosomal protein of 170 kDa isoform X5) c4(343833) c5( ) c6( ) c7(V); #c1(CEP170) c2(XP_011544747) c3(centrosomal protein of 170 kDa isoform X6) c4(343834) c5( ) c6( ) c7(V); #c1(CEP170) c2(XP_011544749) c3(centrosomal protein of 170 kDa isoform X8) c4(343835, 343836) c5( ) c6( ) c7(V); #c1(CEP19) c2(NP_116287) c3(centrosomal protein of 19 kDa) c4(226087) c5(226088, 226089, 226090, 226091, 226092, 226093) c6( ) c7(akQ, v, qM); #c1(CEP192) c2(NP_115518) c3(centrosomal protein of 192 kDa) c4(223035, 223036, 223037) c5(223038, 223039, 223040, 223041, 223042, 223043, 223044, 223045, 223046, 223047, 223048, 223049, 223050, 223051, 223052, 223053, 223054, 223055) c6( ) c7(T); #c1(CEP192) c2(XP_006722390) c3(centrosomal protein of 192 kDa isoform X7) c4(294689, 294690) c5(294691, 294692, 294693, 294694, 294695, 294696) c6( ) c7(T); #c1(CEP192) c2(XP_011523978) c3(centrosomal protein of 192 kDa isoform X11) c4( ) c5(318523, 318524, 318525, 318526, 318527, 318528) c6( ) c7(T); #c1(CEP250) c2(NP_009117) c3(centrosome-associated protein CEP250) c4(170810) c5(170811, 170812, 170813, 170814, 170815, 170816, 170817, 170818, 170819, 170820, 170821, 170822) c6( ) c7(T, et, mG, aT, dz, nw, bX, oc); #c1(CEP250) c2(XP_006723757) c3(centrosome-associated protein CEP250 isoform X2) c4(295360) c5( ) c6( ) c7(T, et, mG, aT, dz, nw, bX, oc); #c1(CEP250) c2(XP_006723758) c3(centrosome-associated protein CEP250 isoform X3) c4(295361, 295362, 295363) c5(295364, 295365, 295366, 295367, 295368, 295369) c6( ) c7(T, et, mG, aT, dz, nw, bX, oc); #c1(CEP250) c2(XP_011526819) c3(centrosome-associated protein CEP250 isoform X6) c4(321136, 321137) c5( ) c6( ) c7(T, et, mG, aT, dz, nw, bX, oc); #c1(CEP250) c2(XP_011526822) c3(centrosome-associated protein CEP250 isoform X10) c4( ) c5(321138, 321139, 321140, 321141, 321142, 321143, 321144, 321145, 321146, 321147, 321148, 321149, 321150, 321151, 321152, 321153, 321154, 321155, 321156, 321157, 321158, 321159, 321160, 321161) c6( ) c7(T, et, mG, aT, dz, nw, bX, oc); #c1(CEP290) c2(NP_079390) c3(centrosomal protein of 290 kDa) c4(217885, 217886) c5(217887, 217888, 217889, 217890, 217891, 217892, 217893, 217894, 217895, 217896, 217897, 217898, 217899, 217900, 217901, 217902, 217903, 217904) c6( ) c7(aKU, arS, nx, vm, Jz, nA, Sp, aKT, cd, Nh, aKV, rz, aKR, ago, Gy, lR, aKS, Tp, nC, bN, U, nw, dy, eQ, de); #c1(CEP290) c2(XP_011537060) c3(centrosomal protein of 290 kDa isoform X2) c4( ) c5(334831) c6( ) c7(aKU, arS, nx, vm, Jz, nA, Sp, aKT, cd, Nh, aKV, rz, aKR, ago, Gy, lR, aKS, Tp, nC, bN, U, nw, dy, eQ, de); #c1(CEP290) c2(XP_011537061) c3(centrosomal protein of 290 kDa isoform X3) c4(334832) c5(334833, 334834, 334835, 334836, 334837, 334838, 334839, 334840, 334841) c6( ) c7(aKU, arS, nx, vm, Jz, nA, Sp, aKT, cd, Nh, aKV, rz, aKR, ago, Gy, lR, aKS, Tp, nC, bN, U, nw, dy, eQ, de); #c1(CEP290) c2(XP_011537064) c3(centrosomal protein of 290 kDa isoform X6) c4( ) c5(334842) c6( ) c7(aKU, arS, nx, vm, Jz, nA, Sp, aKT, cd, Nh, aKV, rz, aKR, ago, Gy, lR, aKS, Tp, nC, bN, U, nw, dy, eQ, de); #c1(CEP290) c2(XP_011537065) c3(centrosomal protein of 290 kDa isoform X7) c4(334843, 334844) c5(334845, 334846, 334847, 334848, 334849, 334850) c6( ) c7(aKU, arS, nx, vm, Jz, nA, Sp, aKT, cd, Nh, aKV, rz, aKR, ago, Gy, lR, aKS, Tp, nC, bN, U, nw, dy, eQ, de); #c1(CEP41) c2(NP_001244087) c3(centrosomal protein of 41 kDa isoform 2) c4(91893) c5(91894, 91895, 91896, 91897, 91898, 91899) c6( ) c7(VX, aKW); #c1(CEP41) c2(NP_001244088) c3(centrosomal protein of 41 kDa isoform 3) c4(91900) c5(91901, 91902, 91903, 91904, 91905, 91906) c6( ) c7(VX, aKW); #c1(CEP41) c2(NP_001244089) c3(centrosomal protein of 41 kDa isoform 4) c4(91907) c5(91908, 91909, 91910, 91911, 91912, 91913, 91914, 91915) c6( ) c7(VX, aKW); #c1(CEP41) c2(NP_061188) c3(centrosomal protein of 41 kDa isoform 1) c4(199885) c5(199886, 199887, 199888, 199889, 199890, 199891) c6( ) c7(VX, aKW); #c1(CEP55) c2(NP_001120654) c3(centrosomal protein of 55 kDa) c4(44395, 44396, 44397) c5(44398, 44399, 44400, 44401, 44402, 44403, 44404, 44405, 44406, 44407, 44408, 44409, 44410, 44411, 44412, 44413, 44414, 44415) c6( ) c7(iu, T, kl, qt, aW, X, r, aT, n, aA, bB, ey, ci, aF, ik, bz, B); #c1(CEP57) c2(NP_001230705) c3(centrosomal protein of 57 kDa isoform b) c4(86618) c5(86619, 86620, 86621, 86622, 86623, 86624, 86625, 86626, 86627, 86628, 86629, 86630) c6( ) c7(akX, T, bP, Ya, aW, aT, ae, ad); #c1(CEP57) c2(NP_001230706) c3(centrosomal protein of 57 kDa isoform c) c4(86631, 86632) c5(86633, 86634, 86635, 86636, 86637, 86638, 86639, 86640, 86641, 86642, 86643, 86644) c6( ) c7(akX, T, bP, Ya, aW, aT, ae, ad); #c1(CEP57) c2(NP_055494) c3(centrosomal protein of 57 kDa isoform a) c4(181504) c5( ) c6( ) c7(akx, T, bP, Ya, aW, aT, ae, ad); #c1(CEP57) c2(XP_006719008) c3(centrosomal protein of 57 kDa isoform X1) c4( ) c5(292158) c6(292159) c7(akX, T, bP, Ya, aW, aT, ae, ad); #c1(CEP63) c2(NP_001035842) c3(centrosomal protein of 63 kDa isoform c) c4(33216) c5(33217, 33218, 33219, 33220, 33221, 33222) c6( ) c7(QK, aKY, xb, id, aii); #c1(CEP63) c2(NP_001035843) c3(centrosomal protein of 63 kDa isoform d) c4(33223) c5(33224, 33225, 33226, 33227, 33228, 33229, 33230, 33231, 33232, 33233, 33234, 33235, 33236, 33237, 33238, 33239, 33240, 33241) c6( ) c7(QK, aKY, xb, id, aii); #c1(CEP63) c2(NP_001035859) c3(centrosomal protein of 63 kDa isoform b) c4(33277) c5(33278, 33279, 33280, 33281, 33282, 33283) c6( ) c7(QK, aKY, xb, id, aii); #c1(CEP63) c2(XP_005247854) c3(centrosomal protein of 63 kDa isoform X1) c4(268008, 268009, 268010) c5(268011, 268012, 268013, 268014, 268015, 268016, 268017, 268018, 268019, 268020) c6( ) c7(QK, aKY, xb, id, aii); #c1(CEP68) c2(NP_055962) c3(centrosomal protein of 68 kDa) c4(184701, 184702, 184703, 184704) c5(184705, 184706, 184707, 184708, 184709, 184710, 184711, 184712, 184713, 184714, 184715, 184716) c6( ) c7(bM, tk); #c1(CEP68) c2(XP_011531006) c3(centrosomal protein of 68 kDa isoform X3) c4( ) c5(326966, 326967, 326968, 326969, 326970, 326971, 326972, 326973, 326974, 326975, 326976, 326977) c6( ) c7(bM, tk); #c1(CEP72) c2(NP_060610) c3(centrosomal protein of 72 kDa) c4(196834, 196835) c5(196836, 196837, 196838, 196839, 196840, 196841, 196842, 196843, 196844, 196845, 196846) c6( ) c7(y, iV); #c1(CEP72) c2(XP_005248380) c3(centrosomal protein of 72 kDa isoform X3) c4( ) c5(268372) c6( ) c7(y, iV); #c1(CEP76) c2(NP_001258918) c3(centrosomal protein of 76 kDa isoform b) c4(96793) c5(96794, 96795, 96796, 96797, 96798, 96799) c6( ) c7(T); #c1(CEP76) c2(NP_079 (75) c3(centrosomal protein of 76 kDa isoform a) c4(217312) c5(217313, 217314, 217315, 217316, 217317, 217318) c6( ) c7(T); #c1(CEP83) c2(NP_057206) c3(centrosomal protein of 83 kDa) c4(189478, 189479) c5(189480, 189481, 189482, 189483, 189484, 189485, 189486, 189487, 189488, 189489, 189490, 189491, 189492, 189493, 189494, 189495) c6( ) c7(aKZ, nA); #c1(CEP85L) c2(NP_001035940) c3(centrosomal protein of 85 kDa-like isoform a) c4(33501) c5(33502, 33503, 33504, 33505, 33506, 33507) c6( ) c7(Nq, Nr, et, e, EQ, dz, Xm, fr); #c1(CEP85L) c2(NP_001171506) c3(centrosomal protein of 85 kDa-like isoform c) c4(70345) c5(70346, 70347, 70348, 70349, 70350, 70351) c6( ) c7(Nq, Nr, et, e, EQ, dz, Xm, fr); #c1(CEP85L) c2(NP_996804) c3(centrosomal protein of 85 kDa-like isoform b) c4(264583) c5(264584, 264585, 264586, 264587, 264588, 264589) c6( ) c7(Nq, Nr, et, e, EI, dz, Xm, fr); #c1(CEP89) c2(NP_116205) c3(centrosomal protein of 89 kDa) c4(225745) c5(225746, 225747, 225748, 225749, 225750, 225751) c6( ) c7(dy); #c1(CER1) c2(NP_005445) c3(cerberus precursor) c4(156269) c5(156270, 156271, 156272, 156273, 156274, 156275) c6( ) c7(dA); #c1(CERK) c2(NP_073603) c3(ceramide kinase) c4(213029) c5(213030, 213031, 213032, 213033, 213034, 213035, 213036) c6( ) c7(aW, aT, n, nk, cm, iu); #c1(CERK) c2(XP_011528617) c3(ceramide kinase isoform X1) c4(323879) c5(323880, 323881, 323882, 323883, 323884) c6( ) c7(aW, aT, n, nk, cm, iu); #c1(CERKL) c2(NP_001025482) c3(ceramide kinase-like protein isoform 2) c4(29604) c5(29605, 29606, 29607, 29608, 29609, 29610) c6( ) c7(cF, lR, nx, nw, nk, nC, ala); #c1(CERKL) c2(NP_001025483) c3(ceramide kinase-like protein isoform 3) c4(29611) c5(29612, 29613, 29614, 29615, 29616, 29617) c6( ) c7(cF, lR, nx, nw, nk, nC, ala); #c1(CERKL) c2(NP_001025484) c3(ceramide kinase-like protein isoform 4) c4(29618) c5(29619, 29620, 29621, 29622, 29623, 29624) c6( ) c7(cF, IR, nx, nw, nk, nC, ala); #c1(CERKL) c2(NP_001153749) c3(ceramide kinase-like protein isoform 7) c4(60978) c5(60979, 60980, 60981, 60982, 60983, 60984) c6( ) c7(cF, lR, nx, nw, nk, nC, ala); #c1(CERKL) c2(NP_963842) c3(ceramide kinase-like protein isoform 1) c4(262419) c5(262420, 262421, 262422, 262423, 262424, 262425) c6( ) c7(cF, lR, nx, nw, nk, nC, aLa); #c1(CERS1) c2(NP_001277194) c3(ceramide synthase 1 isoform 3) c4( ) c5(109217, 109218, 109219, 109220, 109221) c6( ) c7(T, h, fe, K, cm, iK); #c1(CERS1) c2(NP_067090) c3(ceramide synthase 1 isoform 1) c4(208952, 208953, 208954) c5(208955, 208956, 208957, 208958, 208959) c6( ) c7(T, h, fe, K, cm, ik); #c1(CERS1) c2(NP_937850) c3(ceramide synthase 1 isoform 2) c4( ) c5(259294, 259295, 259296, 259297, 259298, 259299, 259300) c6( ) c7(T, h, fe, K, cm, ik); #c1(CERS2) c2(XP_011507753) c3(ceramide synthase 2 isoform X2) c4(297816, 297817, 297818, 297819) c5(297820, 297821, 297822, 297823, 297824, 297825, 297826, 297827, 297828, 297829, 297830) c6( ) c7(BR, aW, Y, aT, aq, dy, ae, B, ad); #c1(CERS3) c2(NP_001277270) c3(ceramide synthase 3 isoform 1) c4(109308, 109309, 109310, 109311) c5(109312, 109313, 109314, 109315, 109316, 109317, 109318, 109319, 109320, 109321, 109322) c6( ) c7(alb, fn, Eb); #c1(CERS3) c2(NP_849164) c3(ceramide synthase 3 isoform 2) c4( ) c5(254378, 254379, 254380, 254381, 254382, 254383, 254384, 254385, 254386, 254387, 254388, 254389, 254390, 254391, 254392, 254393, 254394, 254395, 254396, 254397, 254398) c6( ) c7(alb, fn, Eb); #c1(CERS4) c2(NP_078828) c3(ceramide synthase 4) c4(215633, 215634, 215635, 215636, 215637, 215638) c5(215639, 215640, 215641, 215642, 215643, 215644, 215645, 215646) c6( ) c7(ad); #c1(CERS4) c2(XP_011526590) c3(ceramide synthase 4 isoform X2) c4( ) c5(320896, 320897, 320898, 320899, 320900) c6( ) c7(ad); #c1(CERS4) c2(XP_011526598) c3(ceramide synthase 4 isoform X4) c4( ) c5(320901, 320902, 320903, 320904, 320905, 320906, 320907, 320908, 320909, 320910, 320911, 320912, 320913, 320914, 320915, 320916, 320917, 320918, 320919, 320920, 320921, 320922, 320923, 320924, 320925, 320926, 320927) c6( ) c7(ad); #c1(CERS6) c2(NP_001243055) c3(ceramide synthase 6 isoform 1) c4(89694) c5(89695, 89696, 89697, 89698, 89699, 89700) c6( ) c7(aW, lB, K, aT); #c1(CERS6) c2(NP_982288) c3(ceramide synthase 6 isoform 2) c4(263286) c5(263287, 263288, 263289, 263290, 263291, 263292) c6( ) c7(aW, lB, K, aT); #c1(CESI) c2(NP_001020365) c3(liver carboxylesterase 1 isoform b precursor) c4(28646, 28647) c5(28648, 28649, 28650, 28651, 28652, 28653, 28654, 28655, 28656, 28657, 28658, 28659) c6( ) c7(bL, T, dD, VR, ea, iW, jx, cz, v, j, eq, bX, q, x, av, gd, on, LG, dF, aKB, iU, cH, hl, r, cu, ck, ej, mO, k, bC, bt, B); #c1(CESI) c2(NP_001020366) c3(liver carboxylesterase 1 isoform a precursor) c4(28660) c5(28661, 28662, 28663, 28664, 28665, 28666) c6( ) c7(bL, T, dD, VR, ea, iW, jx, cz, v, j, eq, bX, q, x, av, gd, on, LG, dF, aKB, iU, cH, hl, r, cu, ck, ej, mO, k, bC, bt, B); #c1(CES2) c2(NP_003860) c3(cocaine esterase isoform 1) c4(141574) c5(141575, 141576, 141577, 141578, 141579, 141580) c6( ) c7(gV, T, qX, fM, fV, q, eA, hd, NY, gd, aT, h, LG, md, eH, CT, r, fQ, cu, g, bu, eF, cm, hq, nB, bE, Ei, X); #c1(CES2) c2(NP_932327) c3(cocaine esterase isoform 2) c4(258959) c5(258960, 258961, 258962, 258963, 258964, 258965) c6( ) c7(gV, T, qX, fM, fV, q, eA, hd, NY, gd, aT, h, LG, mQ, eH, CT, r, fQ, cu, g, bu, eF, cm, hq, nB, bE, Ei, X); #c1(CES3) c2(NP_001172105) c3(carboxylesterase 3 isoform 3) c4( ) c5(72396, 72397, 72398, 72399, 72400) c6( ) c7(fQ, ea, dD, j, eq); #c1(CES3) c2(NP_001172106) c3(carboxylesterase 3 isoform 2 precursor) c4(72401) c5(72402, 72403, 72404, 72405, 72406, 72407) c6( ) c7(fQ, ea, dD, j, eq); #c1(CES3) c2(NP_079198) c3(carboxylesterase 3 isoform 1 precursor) c4(217451, 217452) c5(217453, 217454, 217455, 217456, 217457, 217458) c6( ) c7(fQ, ea, dD, j, eq); #c1(CES3) c2(XP_011521277) c3(carboxylesterase 3 isoform X1) c4( ) c5(316174) c6( ) c7(fQ, ea, dD, j, eq); #c1(CETN1) c2(NP_004057) c3(centrin-1) c4(143199) c5(143200, 143201, 143202, 143203, 143204, 143205) c6( ) c7(bM, T, Vo, aA, bB, fr, ci, q, ad, Hx, hd, jz, aE, hW, aT, HD, dL, lQ, ae, eL, aW, Y, ev, lb, cr, r, dN, aF, Xk, dw, eE, bz, ar, Dy, bE, z, U, aps, bt, B, L); #c1(CETN2) c2(NP_004335) c3(centrin-2) c4(145245) c5(145246, 145247, 145248, 145249, 145250, 145251) c6( ) c7(ul, bP, CT, aT, hR, as, ip); #c1 (CETN3) c2(NP_004356) c3(centrin-3 isoform 2) c4(145624) c5(145625, 145626, 145627, 145628, 145629, 145630) c6( ) c7(hR, ip, K, as, aT); #c1(CETP) c2(NP_000069) c3(cholesteryl ester transfer protein isoform 1 precursor) c4(1190) c5(1191, 1192, 1193, 1194, 1195, 1196) c6( ) c7(bM, Zz, acz, ea, e, GK, dY, u, dQ, eN, fc, nY, Au, uC, ZB, dD, wh, aw, Wb, aj, alc, TB, Zu, ec, h, jy, dl, aq, cM, bJ, kT, W, dC, cO, xO, Fi, bm, at, B, L, bL, eR, wL, oe, cz, cR, v, j, bX, nS, J, jF, af, eu, bf, r, wA, ac, c, hr, mf, cS, dy, gT, sY, iW, jh, vG, ah, eq, tK, l, ot, ts, vl, uX, nU, cH, cW, eZ, dq, xT, nb, ZD, gZ, aS, ei, vt); #c1(CETP) c2(NP_001273014) c3(cholesteryl ester transfer protein isoform 2 precursor) c4(104/78) c5(104179, 104180, 104181, 104182, 104183, 104184) c6( ) c7(bM, Zz, acz, ea, e, GK, dY, u, dQ, eN, fc, nY, Au, uC, ZB, dD, wh, aw, Wb, aj, alc, TB, Zu, ec, h, jy, dl, aq, cM, bJ, kT, W, dC, cO, xO, Fi, bm, at, B, L, bL, eR, wL, oe, cz, cR, v, j, bX, nS, J, jF, af, eu, bf, r, wA, ac, c, hr, mf, cS, dy, gT, sY, iW, jh, vG, ah, eq, tK, l, ot, ts, vl, uX, nU, cH, cW, eZ, dq, xT, nb, ZD, gZ, aS, ei, vt); #c1(CFAP126) c2(NP_001013647) c3(protein Flattop) c4(25674) c5(25675, 25676, 25677, 25678, 25679, 25680) c6( ) c7(IV); #c1(CFAP36) c2(NP_001269690) c3(cilia- and flagella-associated protein 36 isoform a) c4(102574) c5(102575, 102576, 102577, 102578, 102579, 102580) c6( ) c7(T); #c1(CFAP36) c2(NP_542398) c3(cilia- and flagella-associated protein 36 isoform b) c4(232330) c5(232331, 232332, 232333, 232334, 232335, 232336) c6( ) c7(T); #c1(CFAP44) c2(NP_060808) c3(cilia- and flagella-associated protein 44 isoform 2) c4( ) c5(197921, 197922, 197923, 197924, 197925, 197926, 197927, 197928, 197929, 197930) c6( ) c7(aw); #c1(CFAP44) c2(XP_005247674) c3(cilia- and flagella-associated protein 44 isoform X11) c4(267925) c5(267926, 267927, 267928, 267929, 267930, 267931) c6( ) c7(aw); #c1(CFAP44) c2(XP_011511277) c3(cilia- and flagella-associated protein 44 isoform X2) c4(301770) c5(301771) c6( ) c7(aw); #c1(CFAP44) c2(XP_011511278) c3(cilia- and flagella-associated protein 44 isoform X1) c4(301772, 301773) c5(301774, 301775, 301776, 301777, 301778) c6( ) c7(aw); #c1(CFAP44) c2(XP_011511286) c3(cilia- and flagella-associated protein 44 isoform X12) c4( ) c5(301779, 301780) c6( ) c7(aw); #c1(CFAP52) c2(NP_001074025) c3(cilia- and flagella-associated protein 52 isoform c) c4(36980) c5(36981, 36982, 36983, 36984, 36985, 36986) c6( ) c7(B); #c1(CFAP52) c2(NP_659491) c3(cilia- and flagella-associated protein 52 isoform b) c4(240200, 240201) c5(240202, 240203, 240204, 240205, 240206, 240207, 240208, 240209, 240210, 240211, 240212, 240213) c6( ) c7(B); #c1(CFAP53) c2(NP_659457) c3(cilia- and flagella-associated protein 53) c4(240057) c5(240058, 240059, 240060, 240061, 240062, 240063) c6( ) c7(ald, ha, lQ); #c1(CFAP57) c2(XP_011539101) c3(cilia- and flagella-associated protein 57 isoform X5) c4(337197) c5( ) c6( ) c7(ale); #c1(CFAP58) c2(NP_001008723) c3(coiled-coil domain-containing protein 147) c4(23388, 23389) c5(23390, 23391, 23392, 23393, 23394, 23395, 23396) c6( ) c7(j); #c1(CFAP58) c2(XP_011537679) c3(coiled-coil domain-containing protein 147 isoform X2) c4( ) c5(335222, 335223, 335224, 335225, 335226, 335227, 335228, 335229, 335230) c6( ) c7(j); #c1(CFAP61) c2(NP_056400) c3(cilia- and flagella-associated protein 61 isoform 1) c4(187409, 187410, 187411) c5(187412, 187413, 187414, 187415, 187416, 187417, 187418, 187419, 187420, 187421, 187422, 187423, 187424, 187425, 187426, 187427, 187428, 187429, 187430, 187431) c6( ) c7(cd); #c1(CFAP61) c2(XP_005260749) c3(cilia- and flagella-associated protein 61 isoform X6) c4(276832) c5(276833, 276834, 276835, 276836, 276837, 276838) c6( ) c7(cd); #c1(CFAP61) c2(XP_005260751) c3(cilia- and flagella-associated protein 61 isoform X10) c4(276839) c5(276840, 276841, 276842, 276843, 276844) c6( ) c7(cd); #c1(CFAP61) c2(XP_005260752) c3(cilia- and flagella-associated protein 61 isoform X11) c4(276845, 276846) c5( ) c6( ) c7(cd); #c1(CFAP6I) c2(XP_005260753) c3(cilia- and flagella-associated protein 61 isoform X12) c4( ) c5(276847, 276848, 276849, 276850, 276851, 276852, 276853, 276854, 276855, 276856, 276857, 276858, 276859, 276860, 276861) c6( ) c7(cd); #c1(CFAP61) c2(XP_011527514) c3(cilia- and flagella-associated protein 61 isoform X9) c4(322312, 322313) c5( ) c6( ) c7(cd); #c1(CFAP61) c2(XP_011527515) c3(cilia- and flagella-associated protein 61 isoform X2) c4(322314) c5(322315, 322316, 322317, 322318, 322319, 322320, 322321, 322322, 322323, 322324, 322325, 322326, 322327, 322328, 322329, 322330, 322331, 322332, 322333, 322334, 322335, 322336) c6( ) c7(cd); #c1(CFAP69) c2(NP_001034795) c3(cilia- and flagella-associated protein 69 isoform 1) c4(32288) c5(32289, 32290, 32291, 32292, 32293, 32294, 32295, 32296, 32297, 32298, 32299, 32300, 32301, 32302, 32303, 32304, 32305, 32306) c6( ) c7(fM); #c1(CFAP69) c2(NP_001153610) c3(cilia- and flagella-associated protein 69 isoform 2) c4(60756, 60757) c5(60758, 60759, 60760, 60761, 60762, 60763) c6( ) c7(fM); #c1(CFAP69) c2(XP_011514879) c3(cilia- and flagella-associated protein 69 isoform X2) c4(307632) c5( ) c6( ) c7(fM); #c1(CFAP97) c2(NP_001278962) c3(cilia- and flagella-associated protein 97 isoform 2) c4(111142) c5(111143, 111144, 111145, 111146, 111147, 111148) c6( ) c7(kh, aW, ds, jh, aT, at, zo, K, eA, ae, cd); #c1(CFAP97) c2(XP_011530455) c3(cilia- and flagella-associated protein 97 isoform X1) c4(326319) c5(326320, 326321, 326322, 326323, 326324, 326325) c6( ) c7(kh, aW, ds, jh, aT, at, zo, K, eA, ae, cd); #c1(CFB) c2(NP_001701) c3(complement factor B preproprotein) c4(120127, 120128, 120129, 120130, 120131, (20132) c5(120133, 120134, 120135, 120136, 120137, 120138, 120139, 120140, 120141, 120142, 120143, 120144, 120145, 120146, 120147, 120148, 120149, 120150, 120151, 120152, 120153, 120154, 120155, 120156, 120157, 120158, 120159, 120160, 120161, 120162, 120163, 120164, 120165, 120166, 120167, 120168, 120169, 120170, 120171, 120172, 120173, 120174, 120175, 120176, 120177, 120178, 120179, 120180, 120181, 120182, 120183, 120184, 120185, 120186, 120187, 120188, 120189, 120190, 120191, 120192, 120193, 120194, 120195, 120196, 120197, 120198, 120199, 120200, 120201, 120202, 120203, 120204, 120205, 120206, 120207, 120208, 120209, 120210, 120211, 120212, 120213, 120214, 120215) c6( ) c7(bL, axi, iW, agC, v, alg, j, C, alh, V, x, ql, agD, h, an, acd, db, Um, af, hM, A, Fn, lR, aAg, ZC, cr, eZ, fe, da, pD, iV, Z, Uk, W, nb, axs, alf, dc, bC, GF, y); #c1(CFC1) c2(NP_115934) c3(cryptic protein isoform 1 precursor) c4(224787) c5(224788, 224789, 224790, 224791, 224792, 224793) c6( ) c7(qC, ali, T, jh, hl, dV, bw, gZ, alj, dq, at, dy, K, alk, alm, hp, j, Fq, aln, eA, aLI); #c1(CFC1B) c2(NP_001072998) c3(cryptic family protein (B precursor) c4(35554) c5(35555, 35556, 35557, 35558, 35559, 35560, 35561, 35562) c6(35563, 35564) c7(ali, alo, BT); #c1(CFD) c2(NP_001919) c3(complement factor D preproprotein) c4(122210) c5(122211, 122212, 122213, 122214, 122215, 122216) c6( ) c7(dP, W, alq, hc, oV, iW, aw, alr, alp, pD, j, v, bq); #c1(CFDP1) c2(NP_006315) c3(craniofacial development protein 1) c4(163929) c5(163930, 163931, 163932, 163933, 163934, 163935) c6( ) c7(T, bb, mR, c, Kv, Y, if, oj, U, aU, j, ci, K, qN, y, af); #c1(CFDP1) c2(XP_011521117) c3(craniofacial development protein 1 isoform X2) c4( ) c5(316037) c6( ) c7(T, bb, mR, c, Kv, Y, if, oj, U, aU, j, ci, K, qN, y, af); #c1(CFH) c2(NP_000177) c3(complement factor H isoform a precursor) c4(3853, 3854) c5(3855, 3856, 3857, 3858, 3859, 3860, 3861, 3862, 3863, 3864) c6(3865, 3866, 3867) c7(oO, lD, T, fR, yS, ea, e, alv, aly, bB, fo, HS, ci, aY, bv, AW, dY, M, dV, azm, K, wt, aHg, xa, UM, li, aW, aLH, hl, da, uY, pD, g, uC, Uk, ba, eM, iA, U, cj, vy, nw, aDM, pJ, iu, aLL, aLC, ajy, dD, alx, aw, n, aj, BF, fr, uE, qW, ft, als, cc, ec, ql, jL, h, lG, aLB, aaA, dL, DU, ey, bl, acd, db, Um, A, Ad, ks, fe, ax, aLE, cM, aBs, alw, W, Be, at, oh, va, B, ani, bL, lk, alt, AL, eR, dk, aA, cz, v, j, C, V, aLK, eA, J, agD, aLM, an, ae, af, aLD, s, oV, bg, Pg, aLJ, DR, ac, wO, hc, DW, AH, dy, Kd, gV, Ke, iW, Hc, agC, Jy, eq, DP, adg, ad, BY, aLF, Ae, alu, tT, alz, agB, aLG, oj, oW, vw, vc, lQ, vl, ul, d, lR, hG, aaB, xs, aU, aaP, eZ, dq, aF, S, bz, dr, DT, nb, aHL, axs, bE, arj, aLA, Fw, bC, oQ, GF, lC, vt, rF, X, y, DH); #c1(CFH) c2(NP_001014975) c3(complement factor H isoform b precursor) c4(26077) c5(26078, 26079, 26080, 26081, 26082, 26083, 26084, 26085, 26086, 26087, 26088, 26089, 26090, 26091, 26092, 26093, 26094) c6(26095, 26096, 26097, 26098) c7(oO, ID, T, fR, yS, ea, e, alv, aly, bB, fo, HS, ci, aY, bv, AW, dY, M, dV, azm, K, wt, aHg, xa, UM, li, aW, aLH, hl, da, uY, pD, g, uC, Uk, ba, eM, iA, U, cj, vy, nw, aDM, pJ, iu, aLL, aLC, ajy, dD, alx, aw, n, aj, BF, fr, uE, qW, ft, als, cc, ec, ql, jL, h, lG, aLB, aaA, dL, DU, ey, bl, acd, db, Um, A, Ad, ks, fe, ax, aLE, cM, aBs, alw, W, Be, at, oh, va, B, ani, bL, lk, alt, AL, eR, dk, aA, cz, v, j, C, V, aLK, eA, J, agD, aLM, an, ae, af, aLD, s, oV, bg, Pg, aLJ, DR, ac, wl, hc, DW, AH, dy, Kd, gV, Ke, iW, Hc, agC, Jy, eq, DP, adg, ad, BY, aLF, Ae, alu, tT, alz, agB, aLG, oj, oW, vw, vc, lQ, vl, ul, d, IR, hG, aaB, xs, aU, aaP, eZ, dq, aF, S, bz, dr, DT, nb, aHL, axs, bE, arj, aLA, Fw, bC, oQ, GF, lC, vt, rF, X, y, DH); #c1(CFHR1) c2(NP_002104) c3(complement factor H-related protein 1 precursor) c4(124598) c5(124599, 124600, 124601, 124602, 124603, 124604) c6( ) c7(T, W, ac, eR, ql, ZC, DW, iW, eZ, cz, n, lo, hn, ey, pT, pJ, bt, Uk); #c1(CFHR2) c2(NP_005657) c3(complement factor H-related protein 2 precursor) c4(158321) c5(158322, 158323, 158324, 158325, 158326, 158327, 158328, 158329, 158330, 158331) c6( ) c7(W); #c1(CFHR3) c2(NP_001160096) c3(complement factor H-related protein 3 isoform 2 precursor) c4(65084) c5(65085, 65086, 65087, 65088, 65089, 65090) c6( ) c7(T, W, eR, ql, ZC, DW, eZ, cz, pD, dy, ey, pJ, Uk); #c1(CFHR3) c2(NP_066303) c3(complement factor H-related protein 3 isoform 1 precursor) c4(207153, 207154) c5(207155, 207156, 207157, 207158, 207159, 207160) c6( ) c7(T, W, eR, ql, ZC, DW, eZ, cz, pD, dy, ey, pJ, Uk); #c1(CFHR3) c2(XP_011507398) c3(complement factor H-related protein 3 isoform X1) c4( ) c5(297261, 297262, 297263, 297264, 297265, 297266) c6( ) c7(T, W, eR, ql, ZC, DW, eZ, cz, pD, dy, ey, pJ, Uk); #c1(CFHR4) c2(NP_001188480) c3(complement factor H-related protein 4 isoform 2 precursor) c4(79211) c5(79212, 79213, 79214, 79215, 79216, 79217) c6( ) c7(W, eZ); #c1(CFHR4) c2(NP_006675) c3(complement factor H-related protein 4 isoform 3 precursor) c4(167049, 167050, 167051) c5(167052, 167053, 167054, 167055, 167056, 167057, 167058, 167059, 167060, 167061, 167062, 167063, 167064, 167065, 167066, 167067, 167068, 167069) c6( ) c7(W, eZ); #c1(CFHR5) c2(NP_110414) c3(complement factor H-related protein 5 precursor) c4(220134, 220135) c5(220136, 220137, 220138, 220139, 220140, 220141, 220142, 220143, 220144, 220145, 220146, 220147, 220148, 220149, 220150, 220151) c6(220152, 220153, 220154, 220155) c7(fU, aLO, acF, W, eR, ql, hc, DW, iW, eZ, n, dy, rF, aLN, aY, aLP); #c1(CFI) c2(NP_000195) c3(complement factor 1 preproprotein) c4(4401, 4402, 4403) c5(4404, 4405, 4406, 4407, 4408, 4409, 4410, 4411, 4412, 4413, 4414, 4415, 4416, 4417, 4418, 4419, 4420, 4421) c6( ) c7(T, eR, AL, aA, n, aU, dP, av, aLS, alu, ql, h, aq, afq, Uk, W, afc, Be, aLR, GF, pJ, B, ao, aLL); #c1(CFI) c2(XP_005263033) c3(complement factor 1 isoform X3) c4(278216) c5(278217, 278218, 278219, 278220, 278221, 278222) c6( ) c7(T, eR, AL, aA, n, aU, dP, av, aLS, alu, ql, h, aq, afq, Uk, W, afc, Be, aLR, GF, pJ, B, ao, aLP); #c1(CFL1) c2(NP_005498) c3(cofilin-1) c4(156826, 156827) c5(156828, 156829, 156830, 156831, 156832, 156833, 156834, 156835, 156836, 156837, 156838, 156839, 156840, 156841, 156842, 156843) c6( ) c7(bM, fd, bB, v, ci, hF, ad, ip, dP, yt, aT, By, K, ae, B, aW, Y, g, cm, bw, bz, ck, eM, lp, bE, iS, vt, it, X); #c1(CFL2) c2(NP_068733) c3(cofilin-2 isoform 1) c4(210119) c5(210120, 210121, 210122, 210123, 210124, 210125, 210126, 210127, 210128, 210129) c6( ) c7(Bm, Am, oj, Ad, aLT); #c1(CFLAR) c2(NP_001120656) c3(CASP8 and FADD-like apoptosis regulator isoform 2) c4(44416) c5(44417, 44418, 44419, 44420, 44421, 44422, 44423, 44424, 44425, 44426, 44427, 44428, 44429, 44430, 44431, 44432, 44433, 44434, 44435, 44436, 44437, 44438, 44439, 44440, 44441, 44442, 44443, 44444, 44445, 44446) c6( ) c7(oO, gh, T, bM, fM, aC, if, ha, eX, jh, aA, bB, n, fd, fr, ci, qN, Qp, V, q, br, ad, eA, XW, iu, ib, aE, afQ, aT, Lv, cy, h, dl, eV, nJ, ae, eL, cp, d, apo, je, nO, aW, sp, Y, ayU, eH, ff, r, hQ, aF, en, g, eF, t, aEx, bz, cm, iV, pm, bw, LB, ck, iM, eM, iA, bE, X, ajd, cj, km, hL, m, y, bt, B, hS); #c1(CFLAR) c2(NP_001189445) c3(CASP8 and FADD-like apoptosis regulator isoform 4) c4(79394) c5(79395, 79396, 79397, 79398, 79399, 79400) c6( ) c7(oO, gh, T, bM, fM, aC, if, ha, eX, jh, aA, bB, n, fd, fr, ci, qN, Qp, V, q, br, ad, eA, XW, iu, ib, aE, afQ, aT, Lv, cy, h, dL, eV, nJ, ae, eL, cp, d, apo, je, nO, aW, sp, Y, ayU, eH, ff, r, hQ, aF, en, g, eF, t, aEx, bz, cm, iV, pm, bw, LB, ck, iM, eM, iA, bE, X, ajd, cj, km, hL, m, y, bt, B, hS); #c1(CFLAR) c2(NP_001189446) c3(CASP8 and FADD-like apoptosis regulator isoform 5) c4( ) c5(79401, 79402, 79403, 79404, 79405) c6( ) c7(oO, gh, T, bM, fM, aC, if, ha, eX, jh, aA, bB, n, fd, fr, ci, qN, Qp, V, q, br, ad, eA, XW, iu, ib, aE, afQ, aT, Lv, cy, h, dl, eV, nJ, ae, eL, cp, d, apo, je, nO, aW, sp, Y, ayU, eH, ff, r, hQ, aF, en, g, eF, t, aEx, bz, cm, iV, pm, bw, LB, ck, iM, eM, iA, bE, X, ajd, cj, km, hL, m, y, bt, B, hS); #c1(CFLAR) c2(NP_001189447) c3(CASP& and FADD-like apoptosis regulator isoform 6) c4( ) c5(79406, 79407, 79408, 79409, 79410) c6( ) c7(oO, gh, T, bM, fM, aC, if, ha, ex, jh, aA, bB, n, fd, fr, ci, qN, Ap, V, q, br, ad, eA, XW, iu, ib, aE, afQ, aT, Lv, cy, h, dl, eV, nJ, ae, eL, cp, d, apo, je, nO, aW, sp, Y, ayU, eH, ff, r, hQ, aF, en, g, eF, t, aEx, bz, cm, iV, pm, bw, LB, ck, iM, eM, iA, bE, X, ajd, cj, km, hL, m, y, bt, B, hS); #c1(CFLAR) c2(NP_003870) c3(CASP8 and FADD-like apoptosis regulator isoform 1) c4(141653, 141654) c5(141655, 141656, 141657, 141658, 141659, 141660, 141661, 141662, 141663, 141664, 141665) c6( ) c7(oO, gh, T, bM, fM, aC, if, ha, ex, jh, aA, bB, n, fd, fr, ci, qN, Qp, V, q, br, ad, eA, XW, iu, ib, aE, afQ, aT, Lv, cy, h, dL, eV, nJ, ae, eL, cp, d, apo, je, nO, aW, sp, Y, ayU, eH, ff, r, hQ, aF, en, g, eF, t, aEx, bz, cm, iV, pm, bw, LB, ck, iM, eM, iA, bE, X, ajd, cj, km, hl, m, y, bt, B, hS); #c1(CFLAR) c2(XP_011510402) c3(CASP8 and FADD-like apoptosis regulator isoform X2) c4(300966, 300967, 300968, 300969, 300970) c5(300971, 300972, 300973, 300974, 300975, 300976) c6( ) c7(oO, gh, T, bM, fM, aC, if, ha, eX, jh, aA, bB, n, fd, fr, ci, qN, Qp, V, q, br, ad, eA, XW, iu, ib, aE, afd, aT, Lv, cy, h, dl, eV, nJ, ae, eL, cp, d, apo, je, nO, aW, sp, Y, ayU, eH, ff, r, hQ, aF, en, g, eF, t, aEx, bz, cm, iV, pm, bw, LB, ck, iM, eM, iA, bE, X, ajd, cj, km, hL, m, y, bt, B, hS); #c1(CFP) c2(NP_002612) c3(properdin precursor) c4(130286) c5(130287, 130288, 130289, 130290, 130291, 130292, 130293, 130294, 130295, 130296, 130297, 130298, 130299, 130300) c6(130301, 130302, 130303, 130304) c7(ajy, aly, iD, aLU, bS, aLX, rP, il, iu, AW, h, K, alV, af, B, aq, alW, pD, W, ig, aS, xg, gD); #c1(CFTR) c2(NP_000483) c3(cystic fibrosis transmembrane conductance regulator) c4(9660, 9661) c5(9662, 9663, 9664, 9665, 9666, 9667, 9668, 9669, 9670, 9671, 9672, 9673, 9674, 9675, 9676, 9677, 9678, 9679, 9680, 9681, 9682, 9683, 9684, 9685, 9686, 9687, 9688, 9689, 9690, 9691, 9692, 9693, 9694, 9695, 9696, 9697, 9698, 9699, 9700, 9701, 9702, 9703, 9704, 9705, 9706, 9707, 9708, 9709, 9710, 9711, 9712, 9713, 9714, 9715, 9716, 9717, 9718, 9719, 9720, 9721, 9722, 9723, 9724, 9725, 9726, 9727, 9728, 9729, 9730, 9731, 9732, 9733, 9734, 9735, 9736, 9737, 9738, 9739, 9740, 9741, 9742, 9743, 9744, 9745, 9746, 9747, 9748, 9749, 9750, 9751, 9752, 9753, 9754, 9755, 9756, 9757, 9758, 9759, 9760, 9761, 9762, 9763, 9764, 9765, 9766, 9767, 9768, 9769, 9770, 9771, 9772, 9773, 9774, 9775, 9776) c6(9777, 9778, 9779, 9780, 9781, 9782, 9783) c7(oO, bM, T, aC, fR, if, aMq, aMv, ci, fw, aMB, aMu, aE, lz, aMh, K, aCO, dQ, dF, bc, azh, eL, aa, aEV, aW, qS, mQ, hl, hN, aMF, g, hJ, zV, aLZ, eM, AV, ki, wB, QF, zl, jM, aKr, avB, gU, n, aj, qN, pM, aMd, awq, KL, aMp, b, jz, aME, ju, aMD, adX, cU, aMj, h, dL, wb, aKE, aMi, ajV, aMC, yi, aMe, A, aMm, sp, aMs, ap, aae, fe, kp, aKv, cM, aMg, ar, wk, JM, at, aMc, aAl, lB, B, aMk, bL, Dw, pF, fG, oe, aA, aBo, j, alx, J, q, aMx, eJ, aMb, qp, aT, kf, vH, aMI, yb, aKG, ae, af, le, ND, r, aMA, afq, en, aMf, OC, EJ, dS, cT, YW, aMz, aMr, fD, UF, xb, ao, aMt, ayZ, KV, Kd, am, Me, atS, VT, jN, aMw, dG, Jz, iG, ad, dP, afm, aMn, ds, arV, vB, Yi, MG, mu, ul, ZE, ff, aF, HI, bz, aLY, aMo, DF, dr, TA, aMa, bE, uR, aMy, ei, asz, i, y, sH); #c1(CFTR) c2(XP_011514056) c3(cystic fibrosis transmembrane conductance regulator isoform X3) c4(306591, 306592) c5(306593, 306594, 306595, 306596, 306597, 306598, 306599, 306600, 306601, 306602) c6(306603, 306604) c7(oO, bM, T, aC, fR, if, aMq, aMv, ci, fw, aMB, aMu, aE, lz, aMh, K, aCO, de, dF, bc, azh, eL, aa, aEV, aW, qS, mQ, hl, hN, aMF, g, hJ, zV, aLZ, eM, AV, ki, wB, QF, zl, jM, aKr, avB, gU, n, aj, qN, pM, aMd, awq, KL, aMp, b, jz, aME, ju, aMD, adX, cU, aMj, h, dL, wb, aKE, aMi, ajV, aMC, yi, aMe, A, aMm, sp, aMs, ap, aae, fe, kp, aKv, cM, aMg, ar, wk, JM, at, aMc, aAl, lB, B, aMk, bL, Dw, pF, fG, oe, aA, aBo, j, alx, J, q, aMx, eJ, aMb, qp, aT, kf, vH, aMl, yb, aKG, ae, af, le, ND, r, aMA, afq, en, aMf, OC, EJ, dS, cT, YW, aMz, aMr, fD, UF, xb, ao, aMt, ayZ, kV, Kd, am, Me, atS, VT, jN, aMw, dG, Jz, iG, ad, dP, afm, aMn, ds, arV, vB, Yi, MG, mu, uQ, ZE, ff, aF, HI, bz, aLY, aMo, DF, dr, TA, aMa, bE, uR, aMy, ei, asz, i, y, sH); #c1(CGA) c2(NP_000726) c3 (glycoprotein hormones alpha chain isoform 2 precursor) c4(14911) c5(14912, 14913, 14914, 14915, 14916, 14917, 14918, 14919, 14920, 14921, 14922, 14923, 14924, 14925, 14926, 14927, 14928, 14929, 14930, 14931, 14932, 14933, 14934, 14935, 14936, 14937, 14938, 14939, 14940, 14941, 14942, 14943, 14944, 14945, 14946, 14947, 14948, 14949, 14950, 14951, 14952, 14953, 14954, 14955, 14956, 14957, 14958, 14959, 14960, 14961, 14962, 14963, 14964, 14965, 14966, 14967, 14968, 14969, 14970, 14971, 14972, 14973, 14974, 14975, 14976, 14977, 14978, 14979, 14980, 14981, 14982, 14983, 14984, 14985, 14986, 14987, 14988, 14989, 14990, 14991, 14992, 14993, 14994, 14995, 14996, 14997, 14998, 14999, 15000, 15001, 15002, 15003, 15004, 15005, 15006) c6(15007, 15008, 15009) c7(Zo, aMK, cZ, gV, gU, T, afa, UH, AS, aZ, axd, l, KY, aiC, n, ci, Eh, ad, ft, aew, db, aT, dL, j, dd, fb, nJ, ae, Df, qk, WZ, eL, aeQ, yo, bf, s, aMG, hG, aW, ND, sq, vT, aML, Lm, NH, Pr, aMH, Hu, aMJ, S, Pd, cl, t, KQ, bz, f, cm, bN, qJ, Ew, bP, eM, HK, hl, at, aez, MW, m, be, aMl, Po, AN); #c1(CGB1) c2(NP_203695) c3(choriogonadotropin subunit beta variant 1 precursor) c4(228433) c5(228434, 228435, 228436, 228437, 228438, 228439, 228440) c6(228441, 228442) c7(CN); #c1(CGB2) c2(NP_203696) c3(choriogonadotropin subunit beta variant 2 precursor) c4(228443, 228444) c5(228445, 228446, 228447, 228448, 228449, 228450, 228451, 228452, 228453, 228454, 228455, 228456) c6( ) c7(CN); #c1(CGB5) c2(NP_149032) c3(chorionic gonadotropin, beta polypeptide 5 precursor) c4(226815, 226816, 226817, 226818, 226819) c5(226820, 226821, 226822, 226823, 226824, 226825, 226826, 226827, 226828, 226829, 226830, 226831, 226832, 226833, 226834, 226835, 226836, 226837, 226838, 226839, 226840, 226841, 226842, 226843, 226844, 226845, 226846, 226847, 226848, 226849, 226850, 226851, 226852, 226853, 226854, 226855, 226856, 226857, 226858, 226859, 226860, 226861, 226862, 226863, 226864) c6(226865, 226866, 226867, 226868, 226869, 226870, 226871, 226872, 226873, 226874) c7(cZ, T, afa, UH, if, fr, ci, qN, ad, ft, aT, dL, j, dd, nJ, Df, ae, eL, aed, kl, CN, vT, aW, ff, Hu, en, cl, f, t, KQ, sn, hL, hR, m, KY); #c1(CGB7) c2(XP_011525821) c3(chorionic gonadotropin, beta polypeptide 7 isoform X1) c4(320206, 320207, 320208) c5(320209, 320210, 320211, 320212, 320213, 320214, 320215, 320216, 320217, 320218, 320219, 320220) c6( ) c7(cZ, T, afa, UH, if, fr, ci, qN, ad, ft, aT, dL, jj, dd, nJ, Df, ae, eL, aeQ, CN, vT, aW, ff, Hu, en, cl, f, t, KQ, hL, hR, m, KY); #c1(CGN) c2(XP_005245422) c3(cingulin isoform X1) c4(266540, 266541, 266542) c5(266543, 266544, 266545, 266546, 266547, 266548, 266549, 266550, 266551, 266552, 266553, 266554, 266555, 266556, 266557, 266558, 266559, 266560, 266561, 266562, 266563, 266564) c6( ) c7(bM, ff, en); #c1(CGNL1) c2(NP_116255) c3(cingulin-like protein 1) c4(225998) c5(225999, 226000, 226001, 226002, 226003, 226004) c6( ) c7(bK); #c1(CGRRF1) c2(NP_006559) c3(cell growth regulator with RING finger domain protein 1) c4(166229) c5(166230, 166231, 166232, 166233, 166234, 166235) c6( ) c7(cl, cF, v, hL, an); #c1(CH25H) c2(NP_003947) c3(cholesterol 25-hydroxylase) c4(142384) c5(142385, 142386, 142387, 142388, 142389, 142390) c6( ) c7(at, ea, af); #c1(CHAC1) c2(NP_001136248) c3(glutathione-specific gamma-glutamylcyclotransferase 1 isoform b) c4(54682) c5(54683, 54684, 54685, 54686, 54687, 54688) c6( ) c7(T, aW, Dd, aT, m, t); #c1 (CHAC1) c2(NP_077016) c3(glutathione-specific gamma-glutamylcyclotransferase 1 isoform a) c4(214753) c5(214754, 214755, 214756, 214757, 214758, 214759) c6( ) c7(T, aW, Dd, aT, m, t); #c1(CHAD) c2(XP_011522516) c3(chondroadherin isoform X1) c4( ) c5(317137) c6( ) c7(ff, en); #c1(CHAF1A) c2(NP_005474) c3(chromatin assembly factor 1 subunit A) c4(156506) c5( ) c6( ) c7(T, dB, aW, bE, ps, aT, bB, pl, m, eA, cm, cM, avn, ad); #c1(CHAF1A) c2(XP_011525907) c3(chromatin assembly factor 1 subunit A isoform X1) c4(320315, 320316) c5( ) c6( ) c7(T, dB, aW, bE, ps, aT, bB, pl, m, eA, cm, cM, avn, ad); #c1(CHAF1A) c2(XP_011525908) c3(chromatin assembly factor 1 subunit A isoform X2) c4( ) c5(320317, 320318, 320319, 320320, 320321, 320322, 320323, 320324, 320325, 320326, 320327, 320328) c6( ) c7(T, dB, aW, bE, ps, aT, bB, pl, m, eA, cm, cM, avn, ad); #c1(CHAF1A) c2(XP_011525909) c3(chromatin assembly factor 1 subunit A isoform X3) c4( ) c5(320329, 320330, 320331, 320332, 320333, 320334) c6( ) c7(T, dB, aW, bE, ps, aT, bB, pl, m, eA, cm, cM, avn, ad); #c1(CHAF1B) c2(NP_005432) c3(chromatin assembly factor 1 subunit B) c4(156129) c5(156130, 156131, 156132, 156133, 156134, 156135) c6( ) c7(f, avn, af, aT); #c1(CHAMP1) c2(NP_001157617) c3(chromosome alignment-maintaining phosphoprotein 1) c4(62322) c5(62323, 62324, 62325, 62326, 62327, 62328, 62329, 62330) c6( ) c7(oO, ck, bM, hd, x, c, nB, mx, hD, h, k, g, pM, eE, Kg, kS, eA, EJ); #c1(CHAT) c2(NP_001136401) c3(choline O-acetyltransferase isoform 1) c4(54878, 54879) c5( ) c6( ) c7(rt, bM, xn, aw, bB, aj, bS, ct, ci, zW, abe, J, xC, xm, zT, ib, xc, sO, ahL, Ak, cy, H, af, Gc, Gv, ff, EZ, en, Z, f, cm, wA, hY, xA, akx, bE, xj, dJ, aMM, R, y, xg, L); #c1(CHAT) c2(NP_001136405) c3(choline 0-acetyltransferase isoform 3) c4(54901) c5(54902, 54903, 54904, 54905, 54906, 54907, 54908, 54909, 54910, 54911, 54912, 54913, 54914, 54915, 54916, 54917, 54918, 54919, 54920, 54921, 54922, 54923, 54924, 54925) c6( ) c7(rt, bM, xn, aw, bB, aj, bS, ct, ci, zW, abe, J, xC, xm, zT, ib, xc, sO, ahL, Ak, cy, H, af, Gc, Gv, ff, EZ, en, Z, f, cm, wA, hY, xA, akx, bE, xj, dJ, aMM, R, y, xg, L); #c1 (CHAT) c2(NP_065574) c3(choline O-acetyltransferase isoform 2) c4(204806) c5(204807, 204808, 204809, 204810, 204811, 204812, 204813, 204814) c6( ) c7(rt, bM, xn, aw, bB, aj, bS, ct, ci, zW, abe, J, xC, xm, zT, ib, xc, sO, ahL, Ak, cy, H, af, Gc, Gv, ff, EZ, en, Z, f, cm, wA, hY, xA, akx, bE, xj, dJ, aMM, R, y, xg, L); #c1(CHCHD1) c2(NP_976043) c3(coiled-coil-helix-coiled-coil-helix domain-containing protein 1) c4(262865, 262866) c5(262867, 262868, 262869, 262870, 262871, 262872, 262873, 262874, 262875, 262876, 262877, 262878) c6( ) c7(kC); #c1(CHCHD1D) c2(NP_998885) c3(coiled-coil-helix-coiled-coil-helix domain-containing protein 10, mitochondrial isoform b precursor) c4(266071) c5(266072, 266073, 266074, 266075, 266076, 266077) c6( ) c7(axR, kC, bb, c); #c1(CHCHD3) c2(NP_060282) c3(MICOS complex subunit MIC19) c4(195188) c5(195189, 195190, 195191, 195192, 195193, 195194) c6( ) c7(rz, lB); #c1(CHCHD3) c2(XP_011514666) c3(MICOS complex subunit MIC19 isoform X2) c4(307321) c5(307322, 307323, 307324, 307325, 307326, 307327) c6( ) c7(rz, lB); #c1(CHCHD5) c2(NP_115685) c3(coiled-coil-helix-coiled-coil-helix domain-containing protein 5 isoform a) c4(223541) c5(223542, 223543, 223544, 223545, 223546, 223547) c6( ) c7(Ea); #c1(CHCHD6) c2(NP_115719) c3(MICOS complex subunit MIC25) c4(223708, 223709) c5(223710, 223711, 223712, 223713, 223714, 223715, 223716, 223717, 223718, 223719, 223720, 223721) c6( ) c7(T); #c1(CHCHD7) c2(NP_001011668) c3(coiled-coil-helix-coiled-coil-helix domain-containing protein 7 isoform b) c4(24683, 24684) c5(24685, 24686, 24687, 24688, 24689, 24690, 24691, 24692, 24693, 24694, 24695, 24696, 24697, 24698) c6( ) c7(afb); #c1(CHCHD7) c2(NP_001011671) c3(coiled-coil-helix-coiled-coil-helix domain-containing protein 7 isoform f) c4( ) c5(24699, 24700, 24701, 24702, 24703, 24704, 24705, 24706, 24707, 24708) c6( ) c7(afb); #c1(CHCHD7) c2(NP_077276) c3(coiled-coil-helix-coiled-coil-helix domain-containing protein 7 isoform d) c4(214878, 214879, 214880) c5(214881, 214882, 214883, 214884, 214885, 214886, 214887) c6( ) c7(afb); #c1(CHD1) c2(XP_005271923) c3(chromodomain-helicase-DNA-binding protein 1 isoform X2) c4(283630) c5(283631) c6( ) c7(T, aE, aW, aT, aA, aj, ci, ae, J, ad); #c1(CHD1) c2(XP_005271924) c3(chromodomain-helicase-DNA-binding protein 1 isoform X1) c4(283632) c5(283633, 283634, 283635, 283636, 283637, 283638, 283639, 283640, 283641, 283642, 283643, 283644, 283645, 283646) c6( ) c7(T, aE, aW, aT, aA, aj, ci, ae, J, ad); #c1(CHD1) c2(XP_011541414) c3(chromodomain-helicase-DNA-binding protein 1 isoform X3) c4(340016) c5(340017, 340018, 340019, 340020, 340021, 340022) c6( ) c7(T, aE, aW, aT, aA, aj, ci, ae, J, ad); #c1(CHD2) c2(NP_001036037) c3(chromodomain-helicase-DNA-binding protein 2 isoform 2) c4(33764, 33765) c5(33766, 33767, 33768, 33769, 33770, 33771, 33772, 33773, 33774, 33775, 33776, 33777, 33778, 33779, 33780, 33781, 33782, 33783) c6( ) c7(ha, cz, vH, aMN, lm, nA, avQ); #c1(CHD2) c2(NP_001262) c3(chromodomain-helicase-DNA-binding protein 2 isoform 1) c4(96969, 96970, 96971) c5(96972, 96973, 96974, 96975, 96976, 96977, 96978, 96979, 96980, 96981, 96982, 96983, 96984) c6( ) c7(ha, cz, vH, aMN, Im, nA, avQ); #c1(CHD3) c2(NP_001005271) c3(chromodomain-helicase-DNA-binding protein 3 isoform 3) c4(21259) c5(21260, 21261, 21262, 21263, 21264, 21265) c6( ) c7(nf, ail, aE); #c1(CHD3) c2(NP_001005273) c3(chromodomain-helicase-DNA-binding protein 3 isoform 1) c4(21266) c5(21267, 21268, 21269, 21270, 21271, 21272) c6( ) c7(nf, ail, aE); #c1(CHD3) c2(NP_005843) c3(chromodomain-helicase-DNA-binding protein 3 isoform 2) c4(159813) c5(159814, 159815, 159816, 159817, 159818, 159819, 159820) c6( ) c7(nf, ail, aE); #c1(CHD4) c2(NP_001284482) c3(chromodomain-helicase-DNA-binding protein 4 isoform 2) c4(111874, 111875) c5(111876, 111877, 111878, 111879, 111880, 111881, 111882, 111883, 111884, 111885, 111886, 111887) c6( ) c7(d, hL); #c1(CHD4) c2(XP_006719021) c3(chromodomain-helicase-DNA-binding protein 4 isoform X2) c4(292160, 292161) c5(292162, 292163, 292164, 292165, 292166, 292167, 292168, 292169, 292170, 292171, 292172, 292173, 292174, 292175, 292176, 292177, 292178, 292179, 292180, 292181) c6( ) c7(d, hL); #c1(CHD4) c2(XP_006719025) c3(chromodomain-helicase-DNA-binding protein 4 isoform X6) c4(292182) c5(292183) c6( ) c7(d, hL); #c1(CHD6) c2(NP_115597) c3(chromodomain-helicase-DNA-binding protein 6) c4(223318, 223319, 223320) c5(223321, 223322, 223323, 223324, 223325, 223326, 223327, 223328, 223329, 223330, 223331, 223332, 223333, 223334, 223335, 223336, 223337, 223338) c6( ) c7(sc, T, id, aA, ci, q, ad, eA, dP, ds, aT, dl, aW, Y, ff, r, Ej, EH, aF, en, t, cm, bz, ck, Dy, ky, m, B); #c1(CHD6) c2(XP_005260633) c3(chromodomain-helicase-DNA-binding protein 6 isoform X8) c4( ) c5(276754) c6( ) c7(sc, T, id, aA, ci, q, ad, eA, dP, ds, aT, dL, aW, Y, ff, r, Ej, EH, aF, en, t, cm, bz, ck, Dy, ky, m, B); #c1(CHD6) c2(XP_006723946) c3(chromodomain-helicase-DNA-binding protein 6 isoform X7) c4(295536, 295537) c5(295538, 295539, 295540, 295541, 295542, 295543, 295544, 295545, 295546, 295547, 295548, 295549, 295550) c6( ) c7(sc, T, id, aA, ci, q, ad, eA, dP, ds, aT, dL, aW, Y, ff, r, Ej, EH, aF, en, t, cm, bz, ck, Dy, ky, m, B); #c1(CHD7) c2(NP_060250) c3(chromodomain-helicase-DNA-binding protein 7) c4(195023) c5(195024, 195025, 195026, 195027, 195028, 195029, 195030, 195031, 195032, 195033) c6( ) c7(aMU, dA, aMQ, acz, aMW, RX, atS, bB, n, rr, q, zo, yn, UE, md, aMV, aE, amJ, nX, aMS, aMT, Gc, ahb, avQ, kp, aMO, BE, bQ, dr, kn, y, aMP, aMR, gY, bE, gZ, Km, UF, bD, gT, sH); #c1(CHD7) c2(XP_011515856) c3(chromodomain-helicase-DNA-binding protein 7 isoform X1) c4( ) c5(308834, 308835, 308836, 308837, 308838) c6( ) c7(aMU, dA, aMQ, acz, aMW, RX, atS, bB, n, rr, q, zo, yn, UE, md, aMV, aE, amJ, nX, aMS, aMT, Gc, ahb, avl, kp, aMO, BE, bQ, dr, kn, y, aMP, aMR, gY, bE, gZ, Km, UF, bD, gT, sH); #c1(CHD7) c2(XP_011515858) c3(chromodomain-helicase-DNA-binding protein 7 isoform X3) c4(308839) c5( ) c6( ) c7(aMU, dA, aMQ, acz, aMW, RX, atS, bB, n, rr, q, zo, yn, UE, md, aMV, aE, amJ, nX, aMS, aMT, Gc, ahb, avl, kp, aMO, BE, b, dr, kn, y, aMP, aMR, gY, bE, gZ, Km, UF, bD, gT, SH); #c1(CHD8) c2(NP_001164100) c3(chromodomain-helicase-DNA-binding protein 8 isoform 1) c4(66591) c5(66592, 66593, 66594, 66595, 66596, 66597, 66598, 66599, 66600, 66601, 66602) c6( ) c7(VX, aMX, avl, nA, rz, bN); #c1(CHD8) c2(NP_065971) c3(chromodomain-helicase-DNA-binding protein 8 isoform 2) c4(206524, 206525) c5(206526, 206527, 206528, 206529, 206530, 206531, 206532, 206533, 206534, 206535, 206536) c6( ) c7(VX, aMX, avQ, nA, rz, bN); #c1(CHDH) c2(NP_060867) c3(choline dehydrogenase, mitochondrial) c4(198169, 198170) c5(198171, 198172, 198173, 198174, 198175, 198176) c6( ) c7(bL, T, dD, ahf, eR, atk, ea, acl, e, aw, ard, cz, sM, n, cR, Zv, j, aj, v, bB, eq, nS, cd, J, Na, agd, ec, M, aT, Nc, K, dQ, rz, aW, hl, QK, dr, ac, gY, bE, wE, aMY, gZ, ei, bD, ZW); #c1(CHDH) c2(XP_006713315) c3(choline dehydrogenase, mitochondrial isoform X2) c4( ) c5(287071, 287072, 287073, 287074, 287075, 287076, 287077, 287078, 287079, 287080, 287081, 287082) c6( ) c7(bL, T, dD, ahf, eR, atk, ea, acI, e, aw, ard, cz, sM, n, cR, Zv, j, aj, v, bB, eq, nS, cd, J, Na, agd, ec, M, aT, Nc, K, dQ, rz, aW, hl, QK, dr, ac, gY, bE, wE, aMY, gZ, ei, bD, ZW); #c1(CHDH) c2(XP_011532241) c3(choline dehydrogenase, mitochondrial isoform X1) c4(328295) c5( ) c6( ) c7(bL, T, dD, ahf, eR, atk, ea, acI, e, aw, ard, cz, sM, n, cR, Zv, j, aj, v, bB, eq, nS, cd, J, Na, agd, ec, M, aT, Nc, K, dQ, rz, aW, hl, QK, dr, ac, gY, bE, wE, aMY, gZ, ei, bD, ZW); #c1(CHEK1) c2(NP_001231775) c3(serine/threonine-protein kinase Chkl isoform 2) c4(87483, 87484, 87485) c5(87486, 87487, 87488, 87489, 87490, 87491, 87492, 87493, 87494, 87495, 87496, 87497, 87498, 87499, 87500, 87501, 87502, 87503, 87504, 87505, 87506, 87507, 87508, 87509, 87510, 87511, 87512, 87513, 87514, 87515, 87516, 87517, 87518, 87519, 87520, 87521) c6( ) c7(T, os, aC, if, iW, jh, aA, bB, fd, oZ, ci, bX, qN, q, ad, eA, ip, iu, ft, aE, aEX, Ok, ajv, oW, aT, CO, dL, ey, K, ae, aii, B, aW, WE, Y, aBq, CT, ff, r, af, La, en, cl, g, t, QK, bz, eF, aj, cm, bw, ck, kP, eM, bP, dB, bE, bt, hL, at, CZ, m, bC, aed, X, BA); #c1(CHEK2) c2(NP_001244316) c3(serine/threonine-protein kinase Chk2 isoform d) c4( ) c5(92139, 92140, 92141, 92142, 92143, 92144, 92145, 92146, 92147, 92148, 92149, 92150, 92151, 92152, 92153, 92154, 92155) c6( ) c7(atT, T, aC, bB, ci, ip, bv, aE, Qk, ajv, K, aNc, MO, eL, xQ, zQ, aW, aNb, LZ, g, eF, cm, bw, aj, eM, nF, cj, cX, m, in, dD, ht, n, cd, ft, aMZ, eV, dL, pl, avc, aeU, aq, dB, hk, anZ, hL, tl, aNa, WL, it, AN, B, bL, AS, eR, ex, ew, aA, cz, hy, pd, q, eA, aT, qv, jj, ae, qw, hM, iU, vT, Y, r, aux, eS, en, cl, t, ik, ck, nM, kP, gX, np, dy, rA, bt, HQ, GI, jh, Gn, eq, ad, av, fb, d, BN, BO, Ub, ff, eH, aF, bz, bF, es, bE, afd, bC, oQ, y, aed, X, BA); #c1(CHEK2) c2(NP_009125) c3(serine/threonine-protein kinase Chk2 isoform a) c4(170896, 170897) c5(170898, 170899, 170900, 170901, 170902, 170903, 170904, 170905, 170906, 170907, 170908, 170909, 170910, 170911, 170912, 170913, 170914, 170915, 170916, 170917, 170918, 170919, 170920, 170921, 170922) c6( ) c7(atT, T, aC, bB, ci, ip, bv, aE, Qk, ajv, K, aNc, MO, eL, xQ, zQ, aW, aNb, LZ, g, eF, cm, bw, aj, eM, nF, cj, cX, m, in, dD, ht, n, cd, ft, aMZ, eV, dL, pl, avc, aeU, aq, dB, hk, anZ, hl, tl, aNa, WL, it, AN, B, bL, AS, eR, eX, ew, aA, cz, hy, pd, q, eA, aT, qv, jj, ae, qw, hM, iU, vT, Y, r, aux, eS, en, cl, t, iK, ck, nM, kP, gX, np, dy, rA, bt, HQ, Gl, jh, Gn, eq, ad, av, fb, d, BN, BO, Ub, ff, eH, aF, bz, bF, es, bE, afd, bC, ol, y, aed, X, BA); #c1(CHEK2) c2(NP_665861) c3(serine/threonine-protein kinase Chk2 isoform b) c4(241856, 241857) c5(241858, 241859, 241860, 241861, 241862, 241863, 241864, 241865, 241866, 241867, 241868, 241869, 241870, 241871, 241872, 241873, 241874, 241875, 241876, 241877) c6( ) c7(atT, T, aC, bB, ci, ip, bv, aE, Qk, ajv, K, aNc, MO, eL, xQ, zQ, aW, aNb, LZ, g, eF, cm, bw, aj, eM, nF, cj, cX, m, in, dD, ht, n, cd, ft, aMZ, eV, dL, pl, avc, aeU, aq, dB, hk, anZ, hL, tl, aNa, WL, it, AN, B, bL, AS, eR, eX, ew, aA, cz, hy, pd, q, eA, aT, qv, jj, ae, qw, hM, iU, vT, Y, r, aux, eS, en, cl, t, ik, ck, nM, kP, gX, np, dy, rA, bt, HQ, GI, jh, Gn, eq, ad, av, fb, d, BN, BO, Ub, ff, eH, aF, bz, bF, es, bE, afd, bC, ol, y, aed, X, BA); #c1(CHEK2) c2(XP_006724179) c3(serine/threonine-protein kinase Chk2 isoform X9) c4(295850) c5( ) c6( ) c7(atT, T, aC, bB, ci, ip, bv, aE, Qk, ajv, K, aNc, MO, eL, xQ, zQ, aW, aNb, LZ, g, eF, cm, bw, aj, eM, nF, cj, cX, m, in, dD, ht, n, cd, ft, aMZ, eV, dL, pl, avc, aeU, aq, dB, hk, anZ, hL, tl, aNa, WL, it, AN, B, bL, AS, eR, eX, ew, aA, cz, hy, pd, q, eA, aT, qv, jj, ae, qw, hM, iU, vT, Y, r, aux, eS, en, cl, t, ik, ck, nM, kP, gX, np, dy, rA, bt, HQ, GI, jh, Gn, eq, ad, av, fb, d, BN, BO, Ub, ff, eH, aF, bz, bF, es, bE, afd, bC, ol, y, aed, X, BA); #c1(CHEK2) c2(XP_011528141) c3(serine/threonine-protein kinase Chk2 isoform X2) c4(323258, 323259) c5(323260, 323261, 323262, 323263, 323264, 323265) c6( ) c7(atT, T, aC, bB, ci, ip, bv, aE, Qk, ajv, K, aNc, MO, eL, xQ, zQ, aW, aNb, LZ, g, eF, cm, bw, aj, eM, nF, cj, cX, m, in, dD, ht, n, cd, ft, aMZ, eV, dL, pl, avc, aeU, aq, dB, hk, anZ, hL, tl, aNa, WL, it, AN, B, bL, AS, eR, eX, ew, aA, cz, hy, pd, q, eA, aT, qv, jj, ae, qw, hM, iU, vT, Y, r, aux, eS, en, cl, t, iK, ck, nM, kP, gX, np, dy, rA, bt, HQ, Gl, jh, Gn, eq, ad, av, fb, d, BN, BO, Ub, ff, eH, aF, bz, bF, es, bE, afd, bC, ol, y, aed, X, BA); #c1(CHEK2) c2(XP_011528144) c3(serine/threonine-protein kinase Chk2 isoform X5) c4(323266) c5(323267) c6( ) c7(atT, T, aC, bB, ci, ip, bv, aE, Ok, ajv, K, aNc, MO, eL, xQ, zQ, aW, aNb, LZ, g, eF, cm, bw, aj, eM, nF, cj, cX, m, in, dD, ht, n, cd, ft, aMZ, eV, dL, pl, avc, aeU, aq, dB, hk, anZ, hL, tl, aNa, WL, it, AN, B, bL, AS, eR, eX, ew, aA, cz, hy, pd, q, eA, aT, qv, j, ae, qw, hM, iU, vT, Y, r, aux, eS, en, cl, t, iK, ck, nM, kP, gX, np, dy, rA, bt, HQ, GI, jh, Gn, eq, ad, av, fb, d, BN, BO, Ub, ff, eH, aF, bz, bF, es, bE, afd, bC, oQ, y, aed, X, BA); #c1(CHEK2) c2(XP_011528146) c3(serine/threonine-protein kinase Chk2 isoform X7) c4(323268) c5(323269) c6( ) c7(atT, T, aC, bB, ci, ip, bv, aE, Qk, ajv, K, aNc, MO, eL, xl, zQ, aW, aNb, LZ, g, eF, cm, bw, aj, eM, nF, cj, cX, m, in, dD, ht, n, cd, ft, aMZ, eV, dL, pl, avc, aeI, aq, dB, hk, anZ, hL, tl, aNa, WL, it, AN, B, bL, AS, eR, eX, ew, aA, cz, hy, pd, q, eA, aT, qv, jj, ae, qw, hM, iU, vT, Y, r, aux, eS, en, cl, t, ik, ck, nM, kP, gX, np, dy, rA, bt, HQ, Gl, jh, Gn, eq, ad, av, fb, d, BN, BO, Ub, ff, eH, aF, bz, bF, es, bE, afd, bC, oQ, y, aed, X, BA); #c1(CHERP) c2(NP_006378) c3(calcium homeostasis endoplasmic reticulum protein) c4(164491) c5(164492, 164493, 164494, 164495, 164496, 164497, 164498) c6( ) c7(ck, pr, aNd, axd, jj, aNe, acD, kS, aR); #c1(CHFR) c2(NP_001154816) c3(E3 ubiquitin-protein ligase CHFR isoform 1) c4(61108, 61109) c5(61110, 61111, 6/112, 61113, 61114, 61115) c6( ) c7(T, if, ht, aA, bB, n, ci, qN, q, av, aT, K, hu, aW, ff, r, aF, en, cl, g, t, apx, bz, eM, bE, Qu, hL, m); #c1 (CHFR) c2(NP_00154817) c3(E3 ubiquitin-protein ligase CHFR isoform 2) c4(61116) c5(61117, 61118, 61119, 61120, 61121, 61122) c6( ) c7(T, if, ht, aA, bB, n, ci, qN, q, av, aT, K, hu, aW, ff, r, af, en, cl, g, t, apx, bz, eM, bE, Qu, hL, m); #c1(CHFR) c2(NP_001154818) c3(E3 ubiquitin-protein ligase CHFR isoform 3) c4(61/23, 61124) c5(61125, 61126, 61127, 61128, 61129, 61130, 61131, 61132, 61133, 61134, 61135, 61136) c6( ) c7(T, if, ht, aA, bB, n, ci, qN, q, av, aT, K, hu, aW, ff, r, aF, en, cl, g, t, apx, bz, eM, bE, Qu, hL, m); #c1 (CHFR) c2(NP_001154819) c3(E3 ubiquitin-protein ligase CHFR isoform 5) c4(61137) c5(61138, 61139, 61140, 61141, 61142, 61143, 61144, 61145, 61146, 61147, 61148) c6( ) c7(T, if, ht, aA, bB, n, ci, qN, q, av, aT, K, hu, aW, ff, r, aF, en, cl, g, t, apx, bz, eM, bE, Qu, hL, m); #c1(CHFR) c2(NP_060693) c3(E3 ubiquitin-protein ligase CHFR isoform 4) c4(197214) c5(197215, 197216, 197217, 197218, 197219, 197220, 197221) c6( ) c7(T, if, ht, aA, bB, n, ci, qN, q, av, aT, K, hu, aW, ff, r, af, en, cl, g, t, apx, bz, eM, bE, Qu, hL, m); #c1(CHGA) c2(NP_001288619) c3(chromogranin-A isoform 2 preproprotein) c4(113845, 113846, 113847) c5(113848, 113849, 113850, 113851, 113852, 113853) c6( ) c7(aMK, T, e, l, aiC, ci, aY, aE, pX, SV, K, eL, azh, yo, aW, Lm, g, lm, cm, qJ, eM, Ji, iS, QF, Zo, gU, lT, n, aj, cd, ft, aNj, dl, dx, ajV, A, sq, AQ, ahJ, fe, aMH, aMJ, YE, bP, bo, tl, aez, hR, MW, anx, Pr, AN, bL, AS, eR, uv, cz, tc, v, Eh, J, aew, nv, aT, ae, af, qk, jf, if, bf, s, aMG, aNi, en, dS, aJU, c, aNh, nG, HK, bt, VB, gV, axd, aNk, jh, aNf, Fb, ad, av, db, ji, fb, nJ, Yi, WZ, hG, ff, dq, aF, S, Pd, HI, bz, qa, Ew, AR, sG, aNg, be, ei, y); #c1(CHGA) c2(XP_011544942) c3(chromogranin-A isoform X1) c4(343908, 343909, 343910, 343911, 343912) c5(343913, 343914, 343915, 343916, 343917, 343918, 343919, 343920) c6( ) c7(aMK, T, e, l, aiC, ci, aY, aE, pX, SV, K, eL, azh, yo, aW, Lm, g, lm, cm, qJ, eM, Ji, iS, QF, Zo, gU, lT, n, aj, cd, ft, aNj, dL, dx, ajV, A, sq, AQ, ahJ, fe, aMH, aMJ, YE, bP, bo, tl, aez, hR, MW, anx, Pr, AN, bL, AS, eR, uv, cz, tc, v, Eh, J, aew, nv, aT, ae, af, qk, jf, if, bf, s, aMG, aNi, en, dS, aJU, c, aNh, nG, HK, bt, VB, gV, axd, aNk, jh, aNf, Fb, ad, av, db, ji, fb, nJ, Yi, WZ, hG, ff, dq, aF, S, Pd, HI, bz, qa, Ew, AR, sG, aNg, be, ei, y); #c1(CHGB) c2(NP_001810) c3 (secretogranin-1 precursor) c4(121334, 121335) c5(121336, 121337, 121338, 121339, 121340, 121341, 121342, 121343, 121344, 121345, 121346, 121347) c6( ) c7(T, yo, CN, c, MD, aW, e, cj, aT, cz, hR, kp, bu, cm, bt, af, dS); #c1(CHI3L1) c2(XP_011507410) c3(chitinase-3-like protein 1 isoform X4) c4(297321) c5(297322, 297323, 297324, 297325, 297326, 297327) c6( ) c7(bM, T, ea, e, al, bB, ci, bh, aY, aE, Ma, rw, M, K, aa, aW, az, hl, zC, g, as, Zn, m, hh, aNm, dD, aw, qy, n, aNl, pM, ai, Oe, ft, zw, sp, zb, fe, vU, W, dB, at, eo, B, bL, jx, cz, v, j, C, V, q, eA, aT, cy, yb, r, en, t, wg, ac, wR, cS, ao, aDj, eq, ad, iC, av, F, yt, ji, lQ, ul, d, cH, vj, ff, cr, iV, bz, LB, gk, bE, z, aS, y); #c1(CHIC1) c2(NP_001034929) c3 (cysteine-rich hydrophobic domain-containing protein 1 isoform 1) c4(32390) c5(32391, 32392, 32393, 32394, 32395, 32396) c6( ) c7(t, m); #c1(CHIC1) c2(NP_001287813) c3(cysteine-rich hydrophobic domain-containing protein 1 isoform 2) c4(112917) c5(112918, 112919, 112920, 112921, 112922, 112923) c6( ) c7(t, m); #c1(CHIC2) c2(NP_036242) c3(cysteine-rich hydrophobic domain-containing protein 2) c4(172595) c5(172596, 172597, 172598, 172599, 172600, 172601) c6( ) c7(Nq, ck, Nr, Xm, bt, yR); #c1(CHIT1) c2(NP_001243054) c3(chitotriosidase-1 isoform 2 precursor) c4(89683) c5(89684, 89685, 89686, 89687, 89688, 89689, 89690, 89691) c6(89692, 89693) c7(oO, bM, G, EM, dD, fR, Ju, cz, cl, aj, j, eq, C, pM, J, ad, dV, aT, aNp, aAt, ul, aNo, aW, mQ, aFd, vU, z, cG, at, wR, pw, aNn, bt); #c1(CHIT1) c2(NP_003456) c3(chitotriosidase-1 isoform 1 precursor) c4(138446) c5(138447, 138448, 138449, 138450, 138451, 138452, 138453, 138454) c6(138455, 138456) c7(oO, bM, G, EM, dD, fR, Ju, cz, cl, aj, j, eq, C, pM, J, ad, dV, aT, aNp, aAt, ul, aNo, aW, mQ, aFd, vU, z, cG, at, wR, pw, aNn, bt); #c1(CHKA) c2(NP_997634) c3(choline kinase alpha isoform b) c4(265617) c5(265618, 265619, 265620, 265621, 265622, 265623) c6( ) c7(T, jh, bB, q, eA, ad, Na, aE, aT, h, Nc, K, ae, B, aW, aNq, r, cm, t, bz, eM, lp, bE, PV, m, X); #c1(CHKB) c2(NP_005189) c3(choline/ethanolamine kinase) c4(153780) c5(153781, 153782, 153783, 153784, 153785, 153786) c6( ) c7(ck, cF, eM, yY, xs, aaN, aNr, oY, X, alY); #c1(CHL1) c2(NP_001240317) c3(neural cell adhesion molecule Li-like protein isoform 3 precursor) c4(88602, 88603, 88604) c5(88605, 88606, 88607, 88608, 88609, 88610) c6( ) c7(T, ul, FG, aio, if, Kk, jh, fd, bB, ci, nA, qN, eA, er, ft, hd, cc, nv, K, Xh, aBl, Yi, s, Y, g, Fu, f, cm, bz, bN, avK, iP, es, iA, iT, bE, Qu, ll, m, Xu, adg, gT, bt, xg); #c1(CHL1) c2(XP_006713003) c3(neural cell adhesion molecule LI-like protein isoform X1) c4(286953, 286954, 286955) c5(286956, 286957, 286958, 286959, 286960, 286961) c6( ) c7(T, ul, FG, aio, if, Kk, jh, fd, bB, ci, nA, qN, eA, er, ft, hd, cc, nv, K, Xh, aBl, Yi, s, Y, g, Fu, f, cm, bz, bN, avK, iP, es, iA, iT, bE, Qu, ll, m, Xu, adg, gT, bt, xg); #c1(CHL1) c2(XP_011531597) c3(neural cell adhesion molecule LI-like protein isoform X2) c4(327713) c5(327714, 327715, 327716, 327717, 327718, 327719) c6( ) c7(T, ul, FG, aio, if, Kk, jh, fd, bB, ci, nA, qN, eA, er, ft, hd, cc, nv, K, Xh, aBl, Yi, s, Y, g, Fu, f, cm, bz, bN, avk, iP, es, iA, iT, bE, Qu, ll, m, Xu, adg, gT, bt, xg); #c1(CHL1) c2(XP_011531599) c3(neural cell adhesion molecule Li-like protein isoform X4) c4( ) c5(327720, 327721, 327722, 327723, 327724, 327725, 327726, 327727, 327728, 327729, 327730, 327731, 327732, 327733, 327734, 327735, 327736, 327737, 327738, 327739, 327740, 327741, 327742, 327743) c6( ) c7(T, ul, FG, aio, if, Kk, jh, fd, bB, ci, nA, qN, eA, er, ft, hd, cc, nv, K, Xh, aBl, Yi, s, Y, g, Fu, f, cm, bz, bN, avK, iP, es, iA, iT, bE, Qu, ll, m, Xu, adg, gT, bt, xg); #c1(CHM) c2(NP_000381) c3(rab proteins geranylgeranyltransferase component A 1 isoform a) c4(8328, 8329) c5(8330, 8331, 8332, 8333, 8334, 8335) c6( ) c7(yY, ahX, Gd, bS, ct, eW, q, Gt, an, bA, hZ, AT, xX, lR, eH, r, eF, Z, cm, nC, W, lF, nw, oQ); #c1(CHM) c2(NP_001138886) c3(rab proteins geranylgeranyltransferase component A 1 isoform b) c4(58079) c5(58080, 58081, 58082, 58083, 58084, 58085) c6( ) c7(yY, ahX, Gd, bS, ct, eW, q, Gt, an, bA, hZ, AT, xX, lR, eH, r, eF, Z, cm, nC, W, lF, nw, oQ); #c1(CHM) c2(XP_006724678) c3(rab proteins geranylgeranyltransferase component A 1 isoform X1) c4( ) c5(296250) c6( ) c7(yY, ahX, Gd, bS, ct, eW, q, Gt, an, bA, hZ, AT, xX, lR, eH, r, eF, Z, cm, nC, W, lF, nw, oQ); #c1(CHM) c2(XP_011529141) c3(rab proteins geranylgeranyltransferase component A 1 isoform X2) c4( ) c5(324488, 324489, 324490, 324491, 324492) c6( ) c7(yY, ahX, Gd, bS, ct, eW, q, Gt, an, bA, hZ, AT, xX, lR, eH, r, eF, Z, cm, nC, W, lF, nw, oQ); #c1(CHML) c2(NP_001812) c3(rab proteins geranylgeranyltransferase component A 2) c4(121348) c5(121349, 121350, 121351, 121352, 121353, 121354) c6( ) c7(AT, bM, z, dc, FQ, nw, oc, V); #c1(CHMP1A) c2(NP_001076783) c3(charged multivesicular body protein 1a isoform 1) c4( ) c5(37541, 37542, 37543, 37544, 37545, 37546) c6( ) c7(es, T, aNs, os, aC, UR, oZ, fr, QK); #c1(CHMP1A) c2(NP_002759) c3(charged multivesicular body protein 1a isoform 2) c4(131758) c5(131759, 131760, 131761, 131762, 131763) c6( ) c7(es, T, aNs, os, aC, UR, oZ, fr, QK); #c1(CHMP1A) c2(XP_011521400) c3(charged multivesicular body protein 1a isoform X2) c4( ) c5(316212, 316213) c6( ) c7(es, T, aNs, os, aC, UR, oZ, fr, QK); #c1(CHMP1A) c2(XP_011521401) c3(charged multivesicular body protein 1a isoform X3) c4(316214) c5(316215) c6( ) c7(es, T, aNs, os, aC, UR, oZ, fr, QK); #c1(CHMP1B) c2(NP_065145) c3(charged multivesicular body protein 1b) c4(204255) c5(204256, 204257, 204258, 204259, 204260, 204261) c6( ) c7(awG, qx, oj, bw, EH, bK, fu, fr, awL, kC); #c1(CHMP2B) c2(NP_001231573) c3(charged multivesicular body protein 2b isoform 2) c4( ) c5(87307, 87308, 87309, 87310, 87311, 87312) c6( ) c7(01, ci, PJ, Rc, yw, WY, oW, yu, cy, ey, af, eu, aNu, aaE, cm, BU, bb, ac, c, aNv, wl, U, aNt); #c1(CHMP2B) c2(NP_054762) c3(charged multivesicular body protein 2b isoform 1) c4(177632) c5(177633, 177634, 177635, 177636, 177637, (77638) c6( ) c7(01, ci, PJ, Rc, yw, WY, oW, yu, cy, ey, af, eu, aNu, aaE, cm, BU, bb, ac, c, aNv, wO, U, aNt); #c1(CHMP2B) c2(XP_011531878) c3(charged multivesicular body protein 2b isoform X1) c4(327930) c5( ) c6( ) c7(01, ci, PJ, Rc, yw, WY, oW, yu, cy, ey, af, eu, aNu, aaE, cm, BU, bb, ac, c, aNv, wl, U, aNt); #c1(CHMP3) c2(NP_001005753) c3(charged multivesicular body protein 3 isoform 2) c4( ) c5(22044, 22045, 22046, 22047, 22048) c6( ) c7(av, h, f, ae, eW, ad); #c1(CHMP3) c2(NP_001180446) c3(charged multivesicular body protein 3 isoform 3) c4(74096) c5(74097, 74098, 74099, 74100, 74101, 74102) c6( ) c7(av, h, f, ae, eW, ad); #c1(CHMP3) c2(NP_057163) c3(charged multivesicular body protein 3 isoform 1) c4(189231) c5(189232, 189233, 189234, 189235, 189236, 189237, 189238, 189239) c6( ) c7(av, h, f, ae, eW, ad); #c1(CHMP4A) c2(NP_054888) c3(charged multivesicular body protein 4a) c4(178213) c5(178214, 178215, 178216, 178217, 178218, 178219) c6( ) c7(h, RB); #c1(CHMP4B) c2(NP_789782) c3(charged multivesicular body protein 4b) c4(252280) c5(252281, 252282, 252283, 252284, 252285, 252286) c6( ) c7(h, RB, aNw); #c1(CHMP4C) c2(NP_689497) c3(charged multivesicular body protein 4c) c4(242940) c5(242941, 242942, 242943, 242944, 242945, 242946) c6( ) c7(t, m); #c1(CHMP5) c2(NP_001182465) c3(charged multivesicular body protein 5 isoform 2) c4(74954) c5(74955, 74956, 74957, 74958, 74959, 74960) c6( ) c7(ck, hF, bt); #c1(CHMP5) c2(NP_057494) c3(charged multivesicular body protein 5 isoform 1) c4(191178) c5(191179, 191180, 191181, 191182, 191183, 191184) c6( ) c7(ck, hF, bt); #c1(CHN1) c2(NP_001020372) c3(N-chimaerin isoform 2) c4(28678) c5(28679, 28680, 28681, 28682, 28683, 28684) c6( ) c7(aNx, aNA, dt, aNz, kh, QG, Nk, arH, aHA, aNy, cm); #c1(CHN1) c2(NP_001193531) c3(N-chimaerin isoform 3) c4(82246) c5(82247, 82248, 82249, 82250, 82251, 82252) c6( ) c7(aNx, aNA, dt, aNz, kh, QG, Nk, arH, aHA, aNy, cm); #c1(CHN1) c2(NP_001813) c3(N-chimaerin isoform 1) c4(121355) c5(121356, 121357, 121358, 121359, 121360, 121361) c6( ) c7(aNx, aNA, dt, aNz, kh, QG, Nk, arH, aHA, aNy, cm); #c1(CHN2) c2(NP_001035025) c3(beta-chimaerin isoform 1) c4(32437) c5(32438, 32439, 32440, 32441, 32442, 32443, 32444, 32445, 32446, 32447, 32448, 32449) c6( ) c7(bL, bM, T, aaF, dA, Y, vy, aT, aq, cm); #c1(CHN2) c2(NP_001279998) c3(beta-chimaerin isoform 3) c4(111187) c5(111188, 111189, 111190, 111191, 111192, 111193) c6( ) c7(bL, bM, T, aaF, dA, Y, vy, aT, aq, cm); #c1(CHN2) c2(NP_001279999) c3(beta-chimaerin isoform 4) c4(111194, 111195) c5(111196, 111197, 111198, 111199, 111200, 11/201) c6( ) c7(bL, bM, T, aaF, dA, Y, vy, aT, aq, cm); #c1(CHN2) c2(NP_001280001) c3(beta-chimaerin isoform 6) c4( ) c5(11/203, 111204, 111205, 11/206, 11/207) c6( ) c7(bL, bM, T, aaF, dA, Y, vy, aT, aq, cm); #c1(CHN2) c2(NP_001280002) c3(beta-chimaerin isoform 7) c4(111208, 111209) c5(111210, 111211, 111212, 111213, 111214, 111215) c6( ) c7(bL, bM, T, aaF, dA, Y, vy, aT, aq, cm); #c1(CHN2) c2(NP_001280005) c3(beta-chimaerin isoform 9) c4(111216) c5(111217, 111218, 111219, (11220, 111221, 111222) c6( ) c7(bL, bM, T, aaF, dA, Y, vy, aT, aq, cm); #c1(CHN2) c2(NP_001280009) c3(beta-chimaerin isoform 13) c4(111223) c5(111224, 111225, 111226, 111227, 111228, 111229) c6( ) c7(bL, bM, T, aaF, dA, Y, vy, aT, aq, cm); #c1(CHN2) c2(NP_001280010) c3(beta-chimaerin isoform 14) c4( ) c5(111230, 111231, 111232, 111233, 111234, 111235) c6( ) c7(bL, bM, T, aaF, dA, Y, vy, aT, aq, cm); #c1(CHN2) c2(NP_004058) c3(beta-chimaerin isoform 2) c4(143206) c5(143207) c6( ) c7(bL, bM, T, aaF, dA, Y, vy, aT, aq, cm); #c1(CHN2) c2(XP_011513408) c3(beta-chimaerin isoform X3) c4( ) c5(305639, 305640, 305641, 305642, 305643, 305644) c6( ) c7(bL, bM, T, aaF, dA, Y, vy, aT, aq, cm); #c1(CHN2) c2(XP_011513409) c3(beta-chimaerin isoform X4) c4(305645) c5(305646, 305647, 305648, 305649, 305650, 305651) c6( ) c7(bL, bM, T, aaF, dA, Y, vy, aT, aq, cm); #c1(CHN2) c2(XP_011513411) c3(beta-chimaerin isoform X6) c4(305652) c5( ) c6( ) c7(bL, bM, T, aaF, dA, Y, vy, aT, aq, cm); #c1(CHODL) c2(NP_001191103) c3(chondrolectin isoform b) c4(81001) c5(81002, 81003, 81004, 81005, 81006, 81007, 81008, 81009) c6( ) c7(kf, T, bz, eM); #c1(CHODL) c2(NP_001191104) c3(chondrolectin isoform c) c4(81010) c5(81011, 81012, 81013, 81014, 81015, 81016, 81017, 81018, 81019, 81020, 81021, 81022, 81023, 81024, 81025, 81026, 81027, 81028, 81029) c6( ) c7(kf, T, bz, eM); #c1(CHODL) c2(NP_001191107) c3(chondrolectin isoform d) c4( ) c5(81030, 81031, 81032, 81033, 81034, 81035, 81036, 81037, 81038, 81039, 81040) c6( ) c7(kf, T, bz, eM); #c1(CHODL) c2(NP_079220) c3(chondrolectin isoform a precursor) c4(217488, 217489) c5(217490, 217491, 217492, 217493, 217494, 217495, 217496) c6( ) c7(kf, T, bz, eM); #c1(CHODL) c2(XP_011527755) c3(chondrolectin isoform X3) c4(322742) c5(322743) c6( ) c7(kf, T, bz, eM); #c1(CHODL) c2(XP_011527756) c3(chondrolectin isoform X4) c4( ) c5(322744, 322745, 322746, 322747) c6( ) c7(kf, T, bz, eM); #c1(CHORDC1) c2(NP_001137545) c3(cysteine and histidine-rich domain-containing protein 1 isoform b) c4(56021) c5(56022, 56023, 56024, 56025, 56026, 56027) c6( ) c7(ac, oe, j, an, fq, af); #c1(CHORDC1) c2(NP_036256) c3(cysteine and histidine-rich domain-containing protein 1 isoform a) c4(172669) c5(172670, 172671, 172672, 172673, 172674, 172675) c6( ) c7(ac, oe, j, an, fq, af); #c1(CHP1) c2(NP_009167) c3(calcineurin B homologous protein 1) c4(171228) c5(171229, 171230, 171231, 171232, 171233, 171234) c6( ) c7(T, ya, if, iW, jh, qN, hX, DK, nv, ds, jZ, dV, kH, h, SH, Ou, an, he, CB, je, aHW, Pg, lo, kk, cm, ck, sk, iM, iQ, aNB, cA, bE, NL, aEG, XO, zv, aEN, B, EB); #c1(CHP2) c2(NP_071380) c3(calcineurin B homologous protein 2) c4(211278) c5(211279, 211280, 211281, 211282, 211283, 211284) c6( ) c7(t, ci, T, m); #c1(CHPF) c2(NP_078812) c3(chondroitin sulfate synthase 2 isoform 1) c4(215594, 215595) c5(215596, 215597, 215598, 215599, 215600, 215601, 215602, 215603, 215604, 215605, 215606, 215607) c6( ) c7(B); #c1(CHPT1) c2(NP_064629) c3(cholinephosphotransferase 1) c4(203375, 203376) c5(203377, 203378, 203379, 203380, 203381, 203382, 203383, 203384, 203385, 203386, 203387, 203388) c6( ) c7(gV, T, dA, eP, aNE, aA, fF, n, ti, KO, v, ci, bB, aNG, aNH, fr, q, ad, eA, eW, bv, co, ft, Do, nv, gd, aT, K, aNC, jf, je, iU, hG, aNl, aW, Y, eH, fc, r, gl, dq, aq, aF, en, g, eF, jT, aNF, t, bz, ir, cm, iR, ck, jb, bw, iM, agc, auo, eM, dB, bE, iA, iT, aND, mx, bo, aNJ, m, kS, X); #c1(CHRAC1) c2(NP_059140) c3(chromatin accessibility complex protein 1) c4(193255) c5(193256, 193257, 193258, 193259, 193260, 193261) c6( ) c7(aW, aT); #c1(CHRD) c2(NP_001291401) c3(chordin isoform 2 precursor) c4(115883) c5(115884, 115885, 115886, 115887, 115888, 115889) c6( ) c7(T, agd, w, RX, Kk, Az, aNL, m, t, agY, ZW, B, aNK); #c1(CHRD) c2(NP_003732) c3(chordin isoform 1 precursor) c4(140504, 140505) c5(140506, 140507, 140508, 140509, 140510, 140511, 140512, 140513, 140514, 140515, 140516, 140517) c6( ) c7(T, agd, w, RX, Kk, Az, aNL, m, t, agY, ZW, B, aNK); #c1(CHRD) c2(XP_011511556) c3(chordin isoform X2) c4(302263) c5(302264, 302265, 302266, 302267, 302268, 302269) c6( ) c7(T, agd, w, RX, Kk, Az, aNL, m, t, agY, ZW, B, aNK); #c1(CHRD) c2(XP_011511557) c3(chordin isoform X3) c4(302270) c5(302271, 302272, 302273, 302274, 302275, 302276) c6( ) c7(T, agd, w, RX, Kk, Az, aNL, m, t, agY, ZW, B, aNK); #c1(CHRD) c2(XP_011511558) c3(chordin isoform X1) c4( ) c5(302277) c6( ) c7(T, agd, w, RX, Kk, Az, aNL, m, t, agY, ZW, B, aNK); #c1(CHRDL1) c2(NP_001137455) c3(chordin-like protein 1 isoform 4 precursor) c4(55785, 55786) c5(55787, 55788, 55789, 55790, 55791, 55792, 55793, 55794, 55795, 55796, 55797, 55798) c6( ) c7(d, aNM, h, cy, j, zL); #c1(CHRDL1) c2(NP_660277) c3(chordin-like protein 1 isoform 3 precursor) c4(240600, 240601) c5(240602, 240603, 240604, 240605, 240606, 240607, 240608, 240609, 240610, 240611, 240612, 240613, 240614, 240615, 240616, 240617) c6( ) c7(d, aNM, h, cy, j, zL); #c1(CHRDL1) c2(XP_005262278) c3(chordin-like protein 1 isoform X1) c4(277807) c5(277808, 277809, 277810, 277811, 277812, 277813) c6( ) c7(d, aNM, h, cy, j, zL); #c1(CHRDL1) c2(XP_005262279) c3(chordin-like protein 1 isoform X2) c4(277814) c5(277815, 277816, 277817, 277818, 277819, 277820) c6( ) c7(d, aNM, h, cy, jj, zl); #c1(CHRFAM7A) c2(XP_011520455) c3(CHRNA7-FAM7A fusion protein isoform X3) c4(315349) c5(315350, 315351, 315352, 315353, 315354, 315355, 315356, 315357, 315358, 315359, 315360, 315361) c6( ) c7(ha, VT, aNN, ti, iD, nA, rc, agd, ib, sO, XY, bK, K, rz, af, ak, gl, aga, wA, jb, akq, bN, Cq, bb, aNO, xg); #c1(CHRFAM7A) c2(XP_011520456) c3(CHRNA7-FAM7A fusion protein isoform X1) c4( ) c5(315362, 315363, 315364, 315365, 315366, 315367, 315368, 315369, 315370, 315371, 315372) c6( ) c7(ha, VT, aNN, ti, iD, nA, rc, agd, ib, sO, XY, bK, K, rz, af, ak, gl, aga, wA, jb, akq, bN, Cq, bb, aNO, xg); #c1(CHRM1) c2(XP_011543044) c3(muscarinic acetylcholine receptor M1 isoform X1) c4(341826, 341827, 341828) c5(341829, 341830, 341831, 341832, 341833, 341834, 341835, 341836, 341837, 341838, 341839, 341840, 341841, 341842, 341843, 341844, 341845, 341846, 341847) c6( ) c7(bM, ot, Dy, ac, DK, HP, af, ha, Be, aNP, aNQ, X, eu); #c1(CHRM2) c2(XP_011514073) c3(muscarinic acetylcholine receptor M2 isoform X1) c4(306619) c5(306620, 306621, 306622, 306623, 306624, 306625, 306626, 306627, 306628, 306629, 306630, 306631, 306632, 306633, 306634, 306635, 306636, 306637, 306638, 306639, 306640, 306641, 306642, 306643, 306644, 306645) c6( ) c7(bM, mr, aw, ha, bO, bS, ct, V, uT, XY, bK, bq, he, af, hm, KC, A, anR, Z, FT, aaS, cA, Gs, Ug, Be, mx, gZ, vL, lB); #c1(CHRM3) c2(NP_000731) c3(muscarinic acetylcholine receptor M3) c4(15010, 15011) c5(15012, 15013, 15014, 15015, 15016, 15017, 15018, 15019, 15020, 15021, 15022, 15023, 15024) c6( ) c7(bL, bM, Bl, ha, al, fN, cz, bB, bO, aj, dG, v, V, q, J, bK, aNR, yi, sp, Y, r, jb, bP, cA, bE, Be, gZ, at, sv, X); #c1(CHRM4) c2(NP_000732) c3(muscarinic acetylcholine receptor M4) c4(15025) c5(15026, 15027, 15028, 15029, 15030, 15031) c6( ) c7(aNN, he, cd); #c1(CHRM5) c2(XP_005254198) c3(muscarinic acetylcholine receptor M5 isoform X1) c4(273052, 273053) c5(273054, 273055, 273056, 273057, 273058, 273059, 273060, 273061, 273062, 273063, 273064, 273065, 273066, 273067, 273068, 273069) c6( ) c7(bP, cu, ac); #c1(CHRNA1) c2(NP_000070) c3(acetylcholine receptor subunit alpha isoform b precursor) c4(1197) c5(1198, 1199, 1200, 1201, 1202, 1203) c6( ) c7(hM, xA, e, aFY, XY, aNS, at, aNU, aNT, xl, pd, bz); #c1(CHRNA1) c2(NP_001034612) c3(acetylcholine receptor subunit alpha isoform a precursor) c4(31911) c5(31912, 31913, 31914, 31915, 31916, 31917) c6( ) c7(hM, xA, e, aFY, XY, aNS, at, aNU, aNT, xl, pd, bz); #c1(CHRNA1D) c2(NP_065135) c3(neuronal acetylcholine receptor subunit alpha-10 isoform a precursor) c4(204/28) c5(204129, 204130, 204131, 204132, 204133, 204134) c6( ) c7(XY); #c1(CHRNA2) c2(NP_000733) c3(neuronal acetylcholine receptor subunit alpha-2 isoform 1 precursor) c4(15032) c5(15033, 15034, 15035, 15036, 15037, 15038, 15039, 15040, 15041, 15042) c6( ) c7(XY, ha, uJ, v, bK, aNV, oi, lB, aNX, aNW, FT, hq); #c1(CHRNA3) c2(NP_000734) c3(neuronal acetylcholine receptor subunit alpha-3 isoform 1 precursor) c4(15043) c5(15044, 15045, 15046, 15047, 15048, 15049, 15050, 15051, 15052, 15053) c6( ) c7(T, EM, am, VR, agh, Ol, aA, v, VV, av, XY, aNU, dL, gl, aNZ, af, N, ap, cu, aF, g, f, jb, bz, aNY, eM, Qu, PV, at, lw, lB, iu); #c1(CHRNA3) c2(NP_001160166) c3(neuronal acetylcholine receptor subunit alpha-3 isoform 2 precursor) c4(65174) c5(65175, 65176, 65177, 65178, 65179, 65180) c6( ) c7(T, EM, am, VR, agh, Ol, aA, v, VV, av, XY, aNU, dL, gl, aNZ, af, N, ap, cu, aF, g, f, jb, bz, aNY, eM, Qu, PV, at, lw, lB, iu); #c1(CHRNA3) c2(XP_006720445) c3(neuronal acetylcholine receptor subunit alpha-3 isoform X2) c4( ) c5(293511, 293512, 293513, 293514, 293515, 293516) c6( ) c7(T, EM, am, VR, agh, Ol, aA, v, VV, av, XY, aNU, dL, gl, aNZ, af, N, ap, cu, aF, g, f, jb, bz, aNY, eM, Qu, PV, at, lw, lB, iu); #c1(CHRNA3) c2(XP_011519475) c3(neuronal acetylcholine receptor subunit alpha-3 isoform X1) c4(314144) c5( ) c6( ) c7(T, EM, am, VR, agh, Ol, aA, v, VV, av, XY, aNU, dL, gl, aNZ, af, N, ap, cu, aF, g, f, jb, bz, aNY, eM, Qu, PV, at, lw, lB, iu); #c1(CHRNA4) c2(NP_000735) c3(neuronal acetylcholine receptor subunit alpha-4 isoform 1 precursor) c4(15054, 15055) c5(15056, 15057, 15058, 15059, 15060, 15061, 15062) c6( ) c7(bL, T, FW, agh, Ol, ha, cV, aA, bB, lr, aj, bS, Vd, ct, Jd, arN, iD, ww, cd, J, aR, ft, no, sO, Gb, aT, aU, h, aNU, dL, gl, hg, an, he, eL, xa, af, ak, hM, bO, aOb, agT, Gv, ff, ala, aF, gu, en, g, Z, aOd, f, bz, eE, akq, bN, hq, pH, Cq, ok, xA, eM, bE, Qu, xj, aNX, alc, at, sv, avl, EZ, xg, aNW, L); #c1(CHRNA4) c2(NP_001243502) c3(neuronal acetylcholine receptor subunit alpha-4 isoform 2) c4( ) c5(90972) c6( ) c7(bL, T, FW, agh, Ol, ha, cV, aA, bB, lr, aj, bS, Vd, ct, Jd, arN, iD, ww, cd, J, aR, ft, no, sO, Gb, aT, aU, h, aNU, dL, gl, hg, an, he, eL, xa, af, ak, hM, bO, alb, agT, Gv, ff, aDa, aF, gu, en, g, Z, aOd, f, bz, eE, akq, bN, hq, pH, Cq, ok, xA, eM, bE, Qu, xj, aNX, alc, at, sv, avl, EZ, xg, aNW, L); #c1(CHRNA4) c2(XP_011526826) c3(neuronal acetylcholine receptor subunit alpha-4 isoform X1) c4(321162) c5(321163, 321164, 321165, 321166, 321167, 321168, 321169, 321170, 321171, 321172, 321173, 321174) c6( ) c7(bL, T, FW, agh, Ol, ha, cV, aA, bB, lr, aj, bS, Vd, ct, Jd, arN, iD, ww, cd, J, aR, ft, no, sO, Gb, aT, aU, h, aNU, dL, gl, hg, an, he, eL, xa, af, ak, hM, bO, alb, agT, Gv, ff, ala, aF, gu, en, g, Z, aOd, f, bz, eE, akq, bN, hq, pH, Cq, ok, xA, eM, bE, Qu, xj, aNX, alc, at, sv, avl, EZ, xg, aNW, L); #c1(CHRNA5) c2(NP_000736) c3(neuronal acetylcholine receptor subunit alpha-5 precursor) c4(15063) c5(15064, 15065, 15066, 15067, 15068, 15069) c6( ) c7(T, VR, agh, Gd, v, ci, VV, av, cF, aU, aNU, Jc, Gv, ff, cu, en, g, jb, bz, hq, pH, aNY, at, lB, it, iu); #c1 (CHRNA6) c2(NP_00186208) c3(neuronal acetylcholine receptor subunit alpha-6 isoform 2 precursor) c4(77421) c5(77422, 77423, 77424, 77425, 77426, 77427) c6( ) c7(av, FW, agh, XY, Gb, aNU, cd); #c1(CHRNA6) c2(NP_004189) c3(neuronal acetylcholine receptor subunit alpha-6 isoform 1 precursor) c4(144234) c5(144235, 144236, 144237, 144238, 144239, 144240) c6( ) c7(av, FW, ach, XY, Gb, aNU, cd); #c1(CHRNA7) c2(NP_000737) c3(neuronal acetylcholine receptor subunit alpha-7 isoform 1 precursor) c4(15070, 15071, 15072) c5(15073, 15074, 15075, 15076, 15077, 15078) c6( ) c7(ha, VT, aNN, ti, iD, ci, nA, rc, av, agd, ot, yt, sO, HC, aaw, bK, K, hg, rz, af, na, Bp, ak, axu, Gv, gl, aga, Lk, LI, ale, EZ, wA, f, uC, cm, jb, akq, bN, Cq, aNO, xg, X); #c1(CHRNA7) c2(NP_001177384) c3(neuronal acetylcholine receptor subunit alpha-7 isoform 2 precursor) c4(72727) c5(72728, 72729, 72730, 72731, 72732, 72733) c6( ) c7(ha, VT, aNN, ti, iD, ci, nA, rc, av, agd, ot, yt, sO, HC, aaw, bK, K, hg, rz, af, na, Bp, ak, axu, Gv, gl, aga, Lk, LI, aDe, EZ, wA, f, uC, cm, jb, akq, bN, Cq, aNO, xg, X): #c1(CHRNA7) c2(XP_011545684) c3(neuronal acetylcholine receptor subunit alpha-7 isoform X1) c4(344599) c5(344600, 344601, 344602, 344603, 344604, 344605) c6( ) c7(ha, VT, aNN, ti, iD, ci, nA, rc, av, agd, ot, yt, sO, HC, aaw, bK, K, hg, rz, af, na, Bp, ak, axu, Gv, gl, aga, Lk, Ll, ale, EZ, wA, f, uC, cm, jb, akq, bN, Cq, aNO, xg, X); #c1(CHRNA7) c2(XP_011545686) c3(neuronal acetylcholine receptor subunit alpha-7 isoform X3) c4( ) c5(344606) c6( ) c7(ha, VT, aNN, ti, iD, ci, nA, rc, av, agd, ot, yt, sO, HC, aaw, bK, K, hg, rz, af, na, Bp, ak, axu, Gv, gl, aga, Lk, Ll, ale, EZ, wA, f, uC, cm, jb, akq, bN, Cq, aNO, xg, X); #c1(CHRNA7) c2(XP_011545688) c3(neuronal acetylcholine receptor subunit alpha-7 isoform X4) c4( ) c5(344607, 344608, 344609, 344610, 344611, 344612) c6( ) c7(ha, VT, aNN, ti, iD, ci, nA, rc, av, agd, ot, yt, sO, HC, aaw, bK, K, hg, rz, af, na, Bp, ak, axu, Gv, gl, aga, Lk, LI, ale, EZ, wA, f, uC, cm, jb, akq, bN, Cq, aNO, xg, X); #c1(CHRNB1) c2(NP_000738) c3(acetylcholine receptor subunit beta precursor) c4(15079, 15080) c5(15081, 15082, 15083, 15084, 15085, 15086, 15087, 15088, 15089, 15090, 15091, 15092) c6( ) c7(aNT, alf, aFY, hM, XY); #c1(CHRNB2) c2(NP_000739) c3(neuronal acetylcholine receptor subunit beta-2 precursor) c4(15093) c5(15094, 15095, 15096, 15097, 15098, 15099) c6( ) c7(cw, gD, cF, alg, af, XY, ha, HC, sv, avl, EZ, f, cM, aNX, xa, aNW, bN, hq); #c1(CHRNB3) c2(NP_000740) c3(neuronal acetylcholine receptor subunit beta-3 precursor) c4(15100) c5(15101, 15102, 15103, 15104, 15105, 15106) c6( ) c7(av, Cq, agh, oe, XY, aNU, VV); #c1(CHRNB4) c2(NP_000741) c3(neuronal acetylcholine receptor subunit beta-4 isoform 1 precursor) c4(15107) c5(15108, 15109, 15110, 15/11, 15112, 15113) c6( ) c7(av, Ol, XY, at, aNU, cu, bS, ct, Rm, v, Z, lB, bz, iu); #c1(CHRNB4) c2(NP_001243496) c3(neuronal acetylcholine receptor subunit beta-4 isoform 2 precursor) c4(90942) c5(90943, 90944, 90945, 90946, 90947, 90948) c6( ) c7(av, DI, XY, at, aNU, cu, bS, ct, Rm, v, Z, lB, bz, iu); #c1(CHRND) c2(NP_000742) c3(acetylcholine receptor subunit delta isoform 1 precursor) c4(15114, 15115) c5(15116, 15117, 15118, 15119, 15120, 15121, 15122, 15123, 15124, 15125, 15126, 15127, 15128, 15129, 15130, 15131) c6( ) c7(xA, hM, e, aFY, XY, aNS, aNT); #c1(CHRND) c2(NP_001243586) c3(acetylcholine receptor subunit delta isoform 2 precursor) c4(91100) c5(91101, 91102, 91103, 91104, 91105, 91106) c6( ) c7(xA, hM, e, aFY, XY, aNS, aNT); #c1(CHRNE) c2(NP_000071) c3(acetylcholine receptor subunit epsilon precursor) c4(1204) c5(1205, 1206, 1207, 1208, 1209, 1210) c6( ) c7(hM, alf, xA, alh, Dq, aFY, pd, aNS, aNT, xl, XM); #c1(CHRNG) c2(NP_005190) c3(acetylcholine receptor subunit gamma precursor) c4(153787) c5(153788, 153789, 153790, 153791, 153792, 153793, 153794, 153795, 153796, 153797) c6( ) c7(bv, xA, ali, e, XY, aDx, Vj); #c1(CHST1) c2(NP_003645) c3(carbohydrate sulfotransferase 1 precursor) c4(139888) c5(139889, 139890, 139891, 139892, 139893, 139894) c6( ) c7(jb, sv, rv); #c1(CHST1D) c2(NP_004845) c3(carbohydrate sulfotransferase 10 precursor) c4(150232, 150233, 150234, 150235, 150236, 150237, 150238) c5(150239, 150240, 150241, 150242, 150243, 150244, 150245, 150246, 150247, 150248, 150249, 150250, 150251, 150252, 150253, 150254, 150255, 150256, 150257, 150258, 150259, 150260, 150261, 150262, 150263, 150264, 150265, 150266, 150267, 150268, 150269, 150270, 150271, 150272, 150273, 150274, 150275, 150276, 150277, 150278, 150279, 150280, 150281, 150282, 150283, 150284) c6( ) c7(Y); #c1(CHST11) c2(NP_001167453) c3(carbohydrate sulfotransferase 11 isoform 2) c4(68927) c5(68928, 68929, 68930, 68931, 68932, 68933) c6( ) c7(jv, alj, aW, cj, aT, aj, kv, eg, jb, ad); #c1(CHST11) c2(NP_060883) c3(carbohydrate sulfotransferase 11 isoform 1) c4(198359) c5(198360, 198361, 198362, 198363, 198364, 198365) c6( ) c7(jv, alj, aW, cj, aT, aj, kv, eg, jb, ad); #c1(CHST12) c2(NP_001230724) c3(carbohydrate sulfotransferase (2) c4(86712, 86713) c5(86714, 86715, 86716, 86717, 86718, 86719, 86720, 86721, 86722, 86723, 86724, 86725) c6( ) c7(jv, eg, alj); #c1(CHST13) c2(NP_690849) c3(carbohydrate sulfotransferase 13 precursor) c4(244961, 244962) c5(244963, 244964, 244965, 244966, 244967, 244968, 244969, 244970, 244971, 244972, 244973, 244974) c6( ) c7(jv, eg, alj); #c1(CHST14) c2(NP_569735) c3(carbohydrate sulfotransferase 14) c4(233918) c5(233919, 233920, 233921, 233922, 233923, 233924, 233925, 233926) c6( ) c7(alm, aU, Eh, Az, aDk); #c1(CHST15) c2(NP_001257694) c3(carbohydrate sulfotransferase 15 isoform 2) c4(95578, 95579) c5(95580, 95581, 95582, 95583, 95584, 95585) c6( ) c7(d, j, m, as); #c1(CHST15) c2(NP_056976) c3 (carbohydrate sulfotransferase 15 isoform 1) c4(188283, 188284) c5(188285, 188286, 188287, 188288, 188289, 188290, 188291, 188292, 188293, 188294, 188295, 188296, 188297, 188298, 188299, 188300) c6( ) c7(d, j, m, as); #c1(CHST15) c2(XP_011538159) c3(carbohydrate sulfotransferase 15 isoform X5) c4( ) c5(335793, 335794, 335795, 335796, 335797, 335798) c6( ) c7(d, j, m, as); #c1(CHST2) c2(NP_004258) c3(carbohydrate sulfotransferase 2) c4(144653) c5(144654, 144655, 144656, 144657, 144658, 144659) c6( ) c7(cF, c, eH, sv, ey, g, iV, eF, jb); #c1(CHST3) c2(XP_006718138) c3(carbohydrate sulfotransferase 3 isoform X1) c4(291548) c5(291549, 291550, 291551, 291552, 291553, 291554) c6( ) c7(bL, T, aC, aZ, e, aw, jN, cz, cR, ci, aOr, azl, aOn, ad, Hr, qC, dP, aex, alq, alp, aT, iv, nJ, ae, dF, bA, eG, s, aMG, aW, g, cl, dr, qa, op, vK, vA, ln, hL, aez, m, B, alo); #c1(CHST4) c2(NP_005760) c3(carbohydrate sulfotransferase 4) c4(159135) c5(159136, 159137, 159138, 159139, 159140, 159141, 159142, 159143, 159144, 159145) c6( ) c7(T, als, ac, g, NE); #c1(CHST5) c2(NP_078809) c3(carbohydrate sulfotransferase 5) c4(215574, 215575) c5(215576, 215577, 215578, 215579, 215580, 215581, 215582, 215583, 215584, 215585, 215586, 215587) c6( ) c7(g, bX, NE); #c1(CHST6) c2(XP_011521387) c3(carbohydrate sulfotransferase 6 isoform X1) c4(316196) c5(316197, 316198, 316199, 316200, 316201, 316202, 316203, 316204, 316205, 316206) c6( ) c7(zN, alv, alu, PK, alt); #c1(CHST8) c2(XP_011525528) c3(carbohydrate sulfotransferase 8 isoform X1) c4(319941) c5(319942, 319943, 319944, 319945, 319946, 319947, 319948, 319949, 319950, 319951, 319952, 319953, 319954, 319955) c6( ) c7(v, bM, fq, aFQ); #c1(CHST9) c2(NP_001243245) c3(carbohydrate sulfotransferase 9 isoform 2) c4(90237) c5(90238, 90239, 90240, 90241, 90242, 90243) c6(90244) c7(bt); #c1(CHST9) c2(NP_113610) c3(carbohydrate sulfotransferase 9 isoform 1) c4(221724) c5(221725, 221726, 221727, 221728, 221729, 221730, 221731, 221732) c6(221733, 221734) c7(bt); #c1(CHST9) c2(XP_011524527) c3(carbohydrate sulfotransferase 9 isoform X2) c4( ) c5(319098) c6( ) c7(bt); #c1(CHSY1) c2(NP_055733) c3(chondroitin sulfate synthase 1 precursor) c4(183/74) c5(183175, 183176, 183177, 183178, 183179, 183180) c6( ) c7(aOw, rV); #c1(CHSY3) c2(NP_787052) c3(chondroitin sulfate synthase 3) c4(252019) c5(252020, 252021, 252022, 252023, 252024, 252025) c6( ) c7(aly, alx); #c1(CHTF18) c2(NP_071375) c3(chromosome transmission fidelity protein 18 homolog) c4(211237, 211238) c5(211239, 211240, 211241, 211242, 211243, 211244, 211245, 211246, 211247, 211248, 211249, 211250) c6( ) c7(K); #c1(CHTF18) c2(XP_005255528) c3(chromosome transmission fidelity protein 18 homolog isoform X1) c4(273712) c5(273713, 273714, 273715, 273716, 273717, 273718) c6( ) c7(K); #c1(CHTOP) c2(NP_001193541) c3(chromatin target of PRMT1 protein isoform 2) c4(82255) c5( ) c6( ) c7(pg, w, dz, aw, et); #c1(CHTOP) c2(NP_001231593) c3(chromatin target of PRMT1 protein isoform 3) c4( ) c5(87313, 87314, 87315, 87316, 87317) c6( ) c7(pg, w, dz, aw, et); #c1(CHTOP) c2(NP_056422) c3(chromatin target of PRMT1 protein isoform 1) c4(187531, 187532) c5(187533, 187534, 187535, 187536, 187537, 187538, 187539, 187540, 187541, 187542, 187543, 187544) c6( ) c7(pg, w, dz, aw, et); #c1(CHURC1) c2(NP_001190992) c3(protein Churchill isoform 2) c4(80441) c5(80442, 80443, 80444, 80445, 80446, 80447) c6( ) c7(bN); #c1(CHURC1) c2(NP_001190993) c3(protein Churchill isoform 3) c4(80448) c5(80449, 80450, 80451, 80452, 80453, 80454) c6( ) c7(bN); #c1(CHURC1) c2(NP_660148) c3(protein Churchill isoform 1) c4(240395, 240396) c5(240397, 240398, 240399, 240400, 240401, 240402, 240403, 240404, 240405, 240406, 240407, 240408) c6( ) c7(bN); #c1(CIAD1) c2(NP_004795) c3(probable cytosolic iron-sulfur protein assembly protein CIAO1) c4(149921) c5(149922, 149923, 149924, 149925, 149926, 149927) c6( ) c7(y, aj, J); #c1(CIAPIN1) c2(NP_064709) c3(anamorsin) c4(203521) c5(203522, 203523, 203524, 203525, 203526, 203527) c6( ) c7(ck, T, py, aE, JI, aA, aF, g, B, pl); #c1(CIB1) c2(NP_006375) c3(calcium and integrin-binding protein 1 isoform b) c4(164463) c5(164464, 164465, 164466, 164467, 164468, 164469) c6( ) c7(bM, T, if, fd, bB, ci, Na, Vp, aE, K, hu, eL, Qe, yo, aeR, aW, az, g, as, eF, cm, alg, bw, iP, eM, nB, iA, aJh, cj, m, afA, lT, n, fr, qN, JW, eW, ft, Do, Bj, KH, Ne, h, eV, dL, ey, pl, Oz, kj, aq, yq, PU, iO, KT, bP, hR, avr, B, cn, fM, eX, aA, oc, atp, q, eA, aHy, zn, Vr, aT, j, ae, jf, s, nO, iU, Y, r, lo, en, hf, cl, t, f, ck, rR, iM, bt, Bh, aJc, hS, id, pY, iW, YM, jh, DE, Fb, Fc, iJ, ad, dP, hd, py, db, oW, hP, d, je, ff, eH, aF, VI, bz, es, RV, bE, aS, iu); #c1(CIB2) c2(NP_006374) c3(calcium and integrin-binding family member 2 isoform 1) c4(164456) c5(164457, 164458, 164459, 164460, 164461, 164462) c6( ) c7(T, fM, iW, ml, aj, oc, br, J, ad, er, bv, aOz, kH, aT, jy, hn, dL, K, ey, rz, nJ, ae, eL, gj, je, ZH, s, Or, CN, aW, ff, en, t, bw, bz, aDA, iO, iM, eM, at, m, Bh, B); #c1(CIC) c2(NP_055940) c3(protein capicua homolog isoform CIC-S) c4(184579) c5(184580, 184581, 184582, 184583, 184584, 184585, 184586, 184587) c6( ) c7(T, zX, dk, RX, nA, Fc, q, er, tD, eA, bv, agd, aT, aDh, dx, jf, d, aW, eZ, r, as, cl, aLY, QG, KB, hc, RW, aDB, aiG, y); #c1 (CIC) c2(XP_005258732) c3(protein capicua homolog isoform X11) c4( ) c5(275719) c6( ) c7(T, zX, dk, RX, nA, Fc, q, er, tD, eA, bv, agd, aT, aDh, dx, jf, d, aW, eZ, r, as, cl, aLY, QG, kB, hc, RW, aOB, aiG, y); #c1(CIC) c2(XP_011524967) c3(protein capicua homolog isoform X6) c4( ) c5(319461) c6( ) c7(T, zX, dk, RX, nA, Fc, q, er, tD, eA, bv, agd, aT, aDh, dx, jf, d, aW, eZ, r, as, cl, aLY, QG, kB, hc, RW, aDB, aiG, y); #c1(CIC) c2(XP_011524969) c3(protein capicua homolog isoform X8) c4( ) c5(319462) c6( ) c7(T, zX, dk, RX, nA, Fc, q, er, tD, eA, bv, agd, aT, aDh, dx, jf, d, aW, eZ, r, as, cl, aLY, QG, kB, hc, RW, aOB, aiG, y); #c1 (CIDEB) c2(XP_005267597) c3(cell death activator CIDE-B isoform X1) c4(281081) c5(281082, 281083, 281084, 281085, 281086, 281087, 281088, 281089, 281090, 281091, 281092, 281093, 281094, 281095, 281096, 281097, 281098, 281099) c6( ) c7(ad); #c1(CIDEC) c2(NP_001186480) c3(cell death activator CIDE-3 isoform 2) c4(77683, 77684) c5(77685, 77686, 77687, 77688, 77689, 77690, 77691, 77692, 77693, 77694, 77695) c6( ) c7(LL, fJ, fc, GR, aq, aj, v, cP, aOC, B, J, QI); #c1(CIDEC) c2(NP_001186481) c3(cell death activator CIDE-3 isoform 3) c4( ) c5(77696, 77697, 77698, 77699, 77700, 77701, 77702) c6( ) c7(LL, fJ, fc, GR, aq, aj, v, cP, aOC, B, J, QI); #c1 (CIDEC) c2(NP_001186552) c3(cell death activator CIDE-3 isoform 1) c4(77818, 77819) c5(77820) c6( ) c7(LL, fJ, fc, GR, aq, aj, v, cP, aOC, B, J, QI); #c1(CIDEC) c2(XP_005265433) c3(cell death activator CIDE-3 isoform X2) c4( ) c5(279541, 279542, 279543, 279544, 279545, 279546, 279547, 279548, 279549) c6( ) c7(LL, fJ, fc, GR, aq, aj, v, cP, aOC, B, J, QI); #c1(CIITA) c2(NP_000237) c3(MHC class II transactivator isoform 2) c4(5321, 5322) c5(5323, 5324, 5325, 5326, 5327, 5328, 5329, 5330, 5331, 5332, 5333, 5334, 5335) c6( ) c7(T, fM, dA, fR, eR, aw, eX, jh, cz, fd, n, cl, cd, jE, ci, V, pd, aDI, eA, hX, bv, py, aX, nX, apJ, NS, aT, h, eV, hK, af, eu, bA, ul, A, aW, Y, oV, ff, eZ, r, fe, hp, NR, en, eE, cm, iV, iP, ac, aOD, gX, eo, fl, ei, Xa, y, WX, B); #c1(CIITA) c2(NP_001273332) c3(MHC class II transactivator isoform 3) c4(104697) c5(104698, 104699, 104700, 104701, 104702, 104703) c6( ) c7(T, fM, dA, fR, eR, aw, eX, jh, cz, fd, n, cl, cl, jE, ci, V, pd, aDI, eA, hX, bv, py, aX, nX, apJ, NS, aT, h, eV, hK, af, eu, bA, ul, A, aW, Y, oV, ff, eZ, r, fe, hp, NR, en, eE, cm, iV, iP, ac, aOD, gX, eo, fl, ei, Xa, y, WX, B); #c1(CIITA) c2(XP_011520797) c3(MHC class II transactivator isoform X11) c4(315801) c5(315802, 315803, 315804, 315805, 315806, 315807) c6( ) c7(T, fM, dA, fR, eR, aw, eX, jh, cz, fd, n, cl, cl, jE, ci, V, pd, aDI, eA, hX, bv, py, aX, nX, apJ, NS, aT, h, eV, hK, af, eu, bA, ul, A, aW, Y, oV, ff, eZ, r, fe, hp, NR, en, eE, cm, iV, iP, ac, aOD, gX, eo, fl, ei, Xa, y, WX, B); #c1(CILP) c2(NP_003604) c3(cartilage intermediate layer protein 1 preproprotein) c4(139529) c5(139530, 139531, 139532, 139533, 139534, 139535) c6( ) c7(aik, y, aef, rp); #c1(CILP2) c2(NP_694953) c3(cartilage intermediate layer protein 2 precursor) c4(245460) c5(245461, 245462, 245463, 245464, 245465, 245466) c6( ) c7(bL, M); #c1(CINP) c2(NP_116019) c3(cyclin-dependent kinase 2-interacting protein) c4(225220) c5(225221, 225222, 225223, 225224, 225225, 225226, 225227, 225228, 225229, 225230, 225231) c6( ) c7(bP); #c1(CINP) c2(XP_005267810) c3(cyclin-dependent kinase 2-interacting protein isoform X3) c4(281259) c5(281260, 281261, 281262, 281263, 281264) c6( ) c7(bP); #c1(CINP) c2(XP_011535132) c3(cyclin-dependent kinase 2-interacting protein isoform X1) c4(331978) c5(331979, 331980, 331981, 331982, 331983, 331984) c6( ) c7(bP); #c1(CIPC) c2(NP_219494) c3(CLOCK-interacting pacemaker) c4(228609, 228610, 228611, 228612, 228613) c5(228614, 228615, 228616, 228617, 228618, 228619, 228620, 228621, 228622, 228623, 228624, 228625, 228626, 228627, 228628, 228629, 228630, 228631, 228632, 228633, 228634, 228635, 228636, 228637, 228638, 228639, 228640, 228641, 228642, 228643) c6( ) c7(ce); #c1(CIR1) c2(NP_004873) c3(corepressor interacting with RBPJ 1) c4(150452) c5(150453, 150454, 150455, 150456, 150457, 150458) c6( ) c7(cm, aj, J); #c1(CIRBP) c2(NP_001287744) c3(cold-inducible RNA-binding protein isoform 2) c4( ) c5(112778, 112779, 112780, 112781, 112782, 112783, 112784, 112785, 112786, 112787) c6( ) c7(T, iV, VX, iZ, aW, eu, hL, aT, cy, cl, j, K, UN, C, ae, ad, Oh); #c1(CIRBP) c2(NP_001287758) c3(cold-inducible RNA-binding protein isoform 3) c4(112816, 112817) c5(112818, 112819, 112820, 112821, 112822, 112823, 112824, 112825, 112826, 112827, 112828, 112829) c6( ) c7(T, iV, VX, iZ, aW, eu, hL, aT, cy, cl, j, K, UN, C, ae, ad, Oh); #c1(CIRBP) c2(XP_006722700) c3(cold-inducible RNA-binding protein isoform X1) c4(294846) c5( ) c6( ) c7(T, iV, VX, iZ, aW, eu, hl, aT, cy, cl, j, K, UN, C, ae, ad, Oh); #c1 (CIRH1A) c2(NP_116219) c3(cirhin) c4(225823, 225824) c5(225825, 225826, 225827, 225828, 225829, 225830, 225831, 225832, 225833, 225834, 225835, 225836) c6( ) c7(hJ, aDE, jw, aOF); #c1(CISD1) c2(NP_060934) c3(CDGSH iron-sulfur domain-containing protein 1) c4(198770) c5(198771, 198772, 198773, 198774, 198775, 198776) c6( ) c7(bL, aj, aw, J); #c1(CISD2) c2(NP_001008389) c3(CDGSH iron-sulfur domain-containing protein 2) c4(23138) c5(23139, 23140, 23141, 23142, 23143, 23144) c6( ) c7(MR, Ae, adF, aOH, awk, aj, aOG, aOI); #c1(CISH) c2(NP_037456) c3(cytokine-inducible SH2-containing protein isoform 1) c4(175581) c5(175582, 175583, 175584, 175585, 175586, 175587) c6( ) c7(bL, T, dD, id, QO, pf, wu, ea, if, aFc, jh, v, eq, ci, qN, fr, aDJ, AD, V, qW, cd, q, ad, hX, co, Bl, jz, aLS, dV, aT, h, dz, dL, abW, wm, ae, eL, pi, Ft, tw, je, vT, aOK, aW, Y, et, fJ, eH, ff, r, lo, en, aCK, eF, Qt, hq, iM, eD, aOL, aS, m, Hb, eo, y, kS, B, VB, cn); #c1(CISH) c2(NP_659508) c3(cytokine-inducible SH2-containing protein isoform 2) c4(240281) c5(240282, 240283, 240284, 240285, 240286, 240287) c6( ) c7(bL, T, dD, id, QO, pf, wu, ea, if, aFc, jh, v, eq, ci, qN, fr, aDJ, AO, V, qW, cd, q, ad, hX, co, Bl, jz, aLS, dV, aT, h, dz, dL, abW, wm, ae, eL, pi, Ft, tw, je, vT, aOK, aW, Y, et, fJ, eH, ff, r, lo, en, aCK, eF, Qt, hq, iM, eD, aOL, aS, m, Hb, eo, y, kS, B, VB, cn); #c1(CIT) c2(NP_001193928) c3(citron Rho-interacting kinase isoform 1) c4(83222) c5(83223, 83224, 83225, 83226, 83227, 83228) c6( ) c7(anj, akN, aji, Gt, A, an, bO, ta, bS, bK, m, ct, ci, Z, aIL); #c1(CIT) c2(NP_009105) c3(citron Rho-interacting kinase isoform 2) c4(170681) c5(170682, 170683, 170684, 170685, 170686, 170687) c6( ) c7(anj, akN, aji, Gt, A, an, bO, ta, bS, bK, m, ct, ci, Z, aIL); #c1(CITED1) c2(NP_001138357) c3(cbp/p300-interacting transactivator 1 isoform 2) c4(56315, 56316, 56317, 56318, 56319, 56320, 56321) c5(56322, 56323, 56324, 56325, 56326, 56327, 56328, 56329, 56330, 56331, 56332, 56333, 56334, 56335, 56336, 56337, 56338, 56339, 56340, 56341, 56342, 56343, 56344, 56345, 56346, 56347, 56348, 56349, 56350, 56351, 56352, 56353) c6( ) c7(qJ, iO, nM, MS, s, nB, aW, Y, MP, Nn, aT, aOM, aq, ci, er); #c1 (CITED1) c2(NP_001138359) c3(cbp/p300-interacting transactivator 1 isoform 1) c4( ) c5(56354, 56355, 56356, 56357, 56358, 56359, 56360, 56361, 56362, 56363, 56364, 56365, 56366, 56367, 56368, 56369, 56370, 56371, 56372, 56373, 56374, 56375) c6( ) c7(qJ, iO, nM, MS, s, nB, aW, Y, MP, Nn, aT, aOM, aq, ci, er); #c1(CITED2) c2(NP_001161860) c3(cbp/p300-interacting transactivator 2 isoform 1) c4( ) c5(66189, 66190, 66191, 66192, 66193, 66194, 66195, 66196, 66197, 66198, 66199, 66200, 66201, 66202, 66203, 66204, 66205) c6( ) c7(bL, bM, oe, EQ, aA, bB, wo, j, md, Mg, il, aT, iv, bl, eu, aW, iw, aF, eF, zZ, aOO, aON, awC, gY, lp, bE, m, bD, Po, X); #c1(CITED2) c2(NP_001161861) c3(cbp/p300-interacting transactivator 2 isoform 2) c4(66206, 66207) c5(66208, 66209, 66210) c6( ) c7(bL, bM, oe, EQ, aA, bB, wo, j, md, Mg, il, aT, iv, bl, eu, aW, iw, aF, eF, zZ, aOO, aON, awC, gY, lp, bE, m, bD, Po, X); #c1(CIZ1) c2(NP_001124490) c3(cip1-interacting zinc finger protein isoform 4) c4(49149) c5(49150) c6( ) c7(T, aU, aT, Wk, aU, Wm, WI, dx, aOP); #c1(CIZ1) c2(NP_001244904) c3(cip1-interacting zinc finger protein isoform 5) c4(92323) c5(92324, 92325, 92326, 92327, 92328, 92329) c6( ) c7(T, aOQ, aT, Wk, aU, Wm, WI, dx, aOP); #c1(CIZ1) c2(XP_005251945) c3(cip1-interacting zinc finger protein isoform X3) c4(271317, 271318, 271319, 271320, 271321) c5(271322, 271323, 271324, 271325, 271326, 271327, 271328, 271329, 271330, 271331, 271332, 271333, 271334, 271335, 271336, 271337, 271338, 271339, 271340, 271341, 271342, 271343, 271344, 271345, 271346, 271347, 271348, 271349, 271350, 271351, 271352, 271353) c6( ) c7(T, aOQ, aT, Wk, aU, Wm, WI, dx, aOP); #c1(CIZ1) c2(XP_005251948) c3(cip1-interacting zinc finger protein isoform X1) c4(271354, 271355) c5(271356, 271357, 271358, 271359, 271360, 271361, 271362, 271363, 271364, 271365, 271366, 271367) c6( ) c7(T, aU, aT, Wk, aU, Wm, Wl, dx, aDP); #c1(CIZ1) c2(XP_005251949) c3(cip1-interacting zinc finger protein isoform X2) c4( ) c5(271368, 271369, 271370, 271371, 271372, 271373) c6( ) c7(T, aU, aT, Wk, aU, Wm, WI, dx, aOP); #c1(CIZ1) c2(XP_005251950) c3(cip1-interacting zinc finger protein isoform X8) c4( ) c5(271374, 271375, 271376, 271377, 271378, 271379) c6( ) c7(T, aU, aT, Wk, aU, Wm, Wl, dx, aOP); #c1(CIZ1) c2(XP_0067/7102) c3(cip1-interacting zinc finger protein isoform X7) c4(290719, 290720) c5(290721, 290722, 290723, 290724, 290725, 290726, 290727, 290728, 290729, 290730, 290731, 290732) c6( ) c7(T, aOQ, aT, Wk, aU, Wm, Wl, dx, aDP); #c1(CIZ1) c2(XP_011516785) c3(cip1-interacting zinc finger protein isoform X9) c4(310230) c5(310231, 310232, 310233, 310234, 310235, 310236) c6( ) c7(T, aOQ, aT, Wk, aU, Wm, WI, dx, aOP); #c1(CKAP2) c2(NP_001091995) c3(cytoskeleton-associated protein 2 isoform 2) c4(39201, 39202) c5(39203, 39204, 39205, 39206, 39207, 39208, 39209, 39210, 39211, 39212, 39213, 39214) c6( ) c7(T, s, aW, aA, aT, aF, g); #c1(CKAP2) c2(NP_001273616) c3(cytoskeleton-associated protein 2 isoform 4) c4(105442) c5(105443, 105444, 105445, 105446, 105447, 105448) c6( ) c7(T, s, aW, aA, aT, aF, g); #c1(CKAP2) c2(NP_060674) c3(cytoskeleton-associated protein 2 isoform 1) c4(197081) c5(197082, 197083, 197084, 197085, 197086, 197087) c6( ) c7(T, s, aW, aA, aT, aF, g); #c1(CKAP2L) c2(NP_689728) c3(cytoskeleton-associated protein 2-like isoform 1) c4(243687, 243688) c5(243689, 243690, 243691, 243692, 243693, 243694, 243695, 243696, 243697, 243698, 243699, 243700) c6( ) c7(dP, Tz); #c1(CKAP4) c2(NP_006816) c3(cytoskeleton-associated protein 4) c4(168340) c5(168341, 168342, 168343, 168344, 168345, 168346) c6( ) c7(aDR, bM, T, Fv, if, ci, Na, aDS, aE, arA, pX, K, hu, eL, li, aW, aEI, az, Az, LZ, aOW, OZ, ark, g, eF, eM, nB, aPc, m, sw, zt, iB, ht, n, qN, aPb, ft, amJ, dL, Dz, aDU, aOX, MD, ks, aOT, fM, yk, arN, AO, akG, q, er, aOY, qK, aT, rO, Nc, Tj, avt, aOV, ae, xR, Y, Lf, r, en, bu, t, iK, sS, QG, kP, zu, rA, Mb, zU, ad, aDZ, hd, db, lQ, ff, eH, cr, asV, Ej, bz, atb, dr, LB, adg, aPa); #c1(CKAP5) c2(NP_001008938) c3 (cytoskeleton-associated protein 5 isoform a) c4(23488, 23489, 23490) c5(23491, 23492, 23493, 23494, 23495, 23496, 23497, 23498, 23499, 23500, 23501, 23502, 23503, 23504, 23505, 23506, 23507, 23508, 23509) c6( ) c7(aT); #c1(CKAP5) c2(NP_055571) c3(cytoskeleton-associated protein 5 isoform b) c4(181991, 181992) c5(181993, 181994, 181995, 181996, 181997, 181998, 181999, 182000, 182001, 182002, 182003, 182004, 182005, 182006, 182007) c6( ) c7(aT); #c1(CKB) c2(NP_001814) c3(creatine kinase B-type) c4(121362, 121363, 121364) c5(121365, 121366, 121367, 121368, 121369, 121370, 121371, 121372, 121373, 121374, 121375, 121376, 121377, 121378, 121379, 121380, 121381, 121382) c6( ) c7(T, aw, jx, aA, bB, oY, qW, q, b, ft, af, r, g, t, bz, ck, W, bE, bo, mx, uS, m, i, X); #c1(CKLF) c2(NP_001035228) c3(chemokine-like factor isoform e) c4(32646) c5(32647, 32648, 32649, 32650, 32651, 32652, 32653, 32654) c6(32655, 32656) c7(eE, bM, T, V, iT); #c1 (CKLF) c2(NP_057410) c3(chemokine-like factor isoform c) c4(190603) c5(190604, 190605, 190606, 190607, 190608, 190609, 190610) c6(190611) c7(eE, bM, T, V, lT); #c1(CKLF) c2(NP_058647) c3(chemokine-like factor isoform a) c4(192991, 192992) c5(192993, 192994, 192995, 192996, 192997, 192998, 192999, 193000, 193001, 193002, 193003, 193004, 193005, 193006, 193007, 193008) c6(193009, 193010, 193011, 193012) c7(eE, bM, T, V, iT); #c1(CKLF) c2(NP_857591) c3(chemokine-like factor isoform b) c4(255245) c5(255246, 255247, 255248, 255249, 255250, 255251, 255252) c6(255253) c7(eE, bM, T, V, lT); #c1(CKLF) c2(NP_857592) c3(chemokine-like factor isoform d) c4(255254) c5(255255, 255256, 255257, 255258, 255259, 255260, 255261, 255262) c6(255263, 255264) c7(eE, bM, T, V, lT); #c1(CKM) c2(NP_001815) c3(creatine kinase M-type) c4(121383) c5(121384, 121385, 121386, 121387, 121388, 121389) c6( ) c7(dR, aw, cy, aPe, j, bX, X, aPd); #c1(CKMT1A) c2(XP_005254555) c3(creatine kinase U-type, mitochondrial isoform X2) c4(273245) c5(273246, 273247, 273248, 273249, 273250) c6( ) c7(ev); #c1(CKMT1A) c2(XP_011520030) c3(creatine kinase U-type, mitochondrial isoform X1) c4(314906, 314907, 314908, 314909, 314910, 314911, 314912) c5(314913, 314914, 314915, 314916, 314917, 314918, 314919, 314920, 314921, 314922, 314923, 314924, 314925, 314926, 314927, 314928, 314929, 314930, 314931, 314932, 314933, 314934, 314935, 314936, 314937, 314938, 314939, 314940, 314941, 314942, 314943, 314944, 314945, 314946, 314947, 314948, 314949, 314950, 314951, 314952, 314953, 314954, 314955, 314956, 314957, 314958, 314959, 314960) c6( ) c7(ev); #c1(CKMT1B) c2(XP_011519498) c3(creatine kinase U-type, mitochondrial isoform X2) c4(314145, 314146) c5(314147, 314148, 314149, 314150, 314151, 314152, 314153, 314154, 314155, 314156, 314157, 314158, 314159) c6( ) c7(at, ev); #c1(CKMT1B) c2(XP_011519500) c3(creatine kinase U-type, mitochondrial isoform X3) c4(314160) c5(314161, 314162, 314163, 314164, 314165, 314166, 314167, 314168, 314169, 314170, 314/71) c6( ) c7(at, ev); #c1(CKMT1B) c2(XP_011519501) c3(creatine kinase U-type, mitochondrial isoform X5) c4( ) c5(314/72) c6( ) c7(at, ev); #c1(CKMT2) c2(NP_001093206) c3(creatine kinase S-type, mitochondrial precursor) c4(39933, 39934) c5(39935, 39936, 39937, 39938, 39939, 39940, 39941, 39942, 39943, 39944, 39945, 39946, 39947, 39948, 39949, 39950, 39951, 39952, 39953, 39954) c6( ) c7(T, bt); #c1(CKS1B) c2(NP_001817) c3(cyclin-dependent kinases regulatory subunit 1) c4(121390, 121391, 121392) c5(121393, 121394, 121395, 121396, 121397, 121398, 121399, 121400, 121401, 121402, 121403, 121404, 121405, 121406, 121407, 121408, 121409, 121410) c6( ) c7(T, atC, jh, fd, fr, ci, ad, dP, x, aT, pk, aW, Y, ff, aq, aF, en, iR, alg, bz, amS, aJh, k, B); #c1 (CKS2) c2(NP_001818) c3(cyclin-dependent kinases regulatory subunit 2) c4(121411) c5(121412, 121413, 121414, 121415, 121416, 121417) c6( ) c7(x, T, kl, nM, jz, aA, r, dl, aF, k, eL, q, ad, ip); #c1 (CLASP1) c2(NP_001135745) c3(CLIP-associating protein 1 isoform 2) c4(52991, 52992) c5(52993, 52994, 52995, 52996, 52997, 52998, 52999, 53000, 53001, 53002, 53003, 53004) c6( ) c7(j, aT); #c1(CLASP1) c2(NP_001135746) c3(CLIP-associating protein 1 isoform 3) c4(53005) c5(53006, 53007, 53008, 53009, 53010, 53011) c6( ) c7(j, aT); #c1(CLASP1) c2(NP_001193980) c3(CLIP-associating protein 1 isoform 4) c4(83411) c5(83412, 83413, 83414, 83415, 83416, 83417, 83418) c6( ) c7(j, aT); #c1(CLASP1) c2(NP_056097) c3(CLIP-associating protein 1 isoform 1) c4(185692) c5(185693, 185694, 185695, 185696, 185697, 185698) c6( ) c7(j, aT); #c1(CLASP1) c2(XP_006712448) c3(CLIP-associating protein 1 isoform X20) c4(286659) c5(286660, 286661, 286662, 286663, 286664, 286665) c6( ) c7(j, aT); #c1(CLASP1) c2(XP_011509150) c3(CLIP-associating protein 1 isoform X9) c4(299779, 299780) c5(299781, 299782, 299783, 299784, 299785, 299786, 299787, 299788, 299789, 299790, 299791) c6( ) c7(j, aT); #c1(CLASP2) c2(NP_001193973) c3(CLIP-associating protein 2 isoform 2) c4(83392) c5(83393, 83394, 83395, 83396, 83397, 83398) c6( ) c7(Z, hH); #c1(CLASP2) c2(NP_055912) c3(CLIP-associating protein 2 isoform 1) c4(184459) c5(184460, 184461, 184462, 184463, 184464, 184465) c6( ) c7(Z, hH); #c1(CLASP2) c2(XP_006713115) c3(CLIP-associating protein 2 isoform X21) c4(287019) c5(287020, 287021, 287022, 287023, 287024, 287025) c6( ) c7(Z, hH); #c1(CLASP2) c2(XP_006713116) c3(CLIP-associating protein 2 isoform X22) c4(287026, 287027) c5(287028, 287029, 287030, 287031, 287032, 287033, 287034, 287035, 287036, 287037, 287038, 287039) c6( ) c7(Z, hH); #c1(CLASP2) c2(XP_006713121) c3(CLIP-associating protein 2 isoform X29) c4( ) c5(287040, 287041, 287042, 287043, 287044, 287045) c6( ) c7(Z, hH); #c1(CLASP2) c2(XP_011531823) c3(CLIP-associating protein 2 isoform X27) c4(327888, 327889) c5(327890, 327891, 327892, 327893, 327894, 327895) c6( ) c7(Z, hH); #c1(CLC) c2(NP_001819) c3(galectin-10) c4(121418) c5(121419, 121420, 121421, 121422, 121423, 121424) c6( ) c7(T, XD, aAN, ha, r, cu, ab, aOB, dy, hp, aDp, bt, aLY, q, dr); #c1(CLCA2) c2(XP_011540750) c3(calcium-activated chloride channel regulator 2 isoform X1) c4( ) c5(339327, 339328, 339329, 339330, 339331, 339332) c6( ) c7(T, Qu, aW, aT, aPf, K, ae, ad); #c1(CLCA4) c2(XP_011539317) c3(calcium-activated chloride channel regulator 4 isoform X1) c4(337395) c5(337396, 337397, 337398, 337399, 337400, 337401, 337402, 337403) c6(337404, 337405) c7(aPf, aW, ao, aT); #c1(CLCF1) c2(NP_001159684) c3(cardiotrophin-like cytokine factor 1 isoform 2 precursor) c4(64573) c5(64574, 64575, 64576, 64577, 64578, 64579, 64580, 64581) c6( ) c7(T, aPg, aAN, XD, aPh, ha, r, aOB, dy, aLY, q, dr); #c1(CLCF1) c2(NP_037378) c3(cardiotrophin-like cytokine factor 1 isoform 1 precursor) c4(175152) c5(175153, 175154, 175155, 175156, 175157, 175158, 175159, 175160) c6( ) c7(T, aPg, aAN, XD, aPh, ha, r, aOB, dy, aLY, q, dr); #c1(CLCN1) c2(NP_000074) c3(chloride channel protein 1) c4(1227) c5(1228, 1229, 1230, 1231, 1232, 1233) c6( ) c7(no, zs, aPi, aPk, ha, oj, ao, aOB, avl, aPj, akA, aPl, aPm, aLY, dr, dR); #c1(CLCN2) c2(NP_001164558) c3(chloride channel protein 2 isoform 2) c4(66993) c5(66994, 66995, 66996, 66997, 66998, 66999) c6( ) c7(iV, Cq, aPn, aPo, ha, aPp, aCs, aT, lr, ao, avl, HN, Ji, hg, eA, iD, cb, ji, akq, aR); #c1(CLCN2) c2(NP_001164559) c3(chloride channel protein 2 isoform 3) c4(67000) c5(67001, 67002, 67003, 67004, 67005, 67006, 67007) c6( ) c7(iV, Cq, aPn, aPo, ha, aPp, aCs, aT, lr, ao, avl, HN, Ji, hg, eA, iD, cb, ji, akq, aR); #c1(CLCN2) c2(NP_001164560) c3(chloride channel protein 2 isoform 4) c4(67008) c5(67009, 67010, 67011, 67012, 67013, 67014) c6( ) c7(iV, Cq, aPn, aPo, ha, aPp, aCs, aT, lr, ao, avl, HN, Ji, hg, eA, iD, cb, ji, akq, aR); #c1(CLCN2) c2(NP_004357) c3(chloride channel protein 2 isoform 1) c4(145631) c5(145632, 145633, 145634, 145635, 145636, 145637, 145638, 145639) c6( ) c7(iV, Cq, aPn, aPo, ha, aPp, aCs, aT, lr, ao, avl, HN, Ji, hg, eA, iD, cb, ji, akq, aR); #c1(CLCN2) c2(XP_011510704) c3(chloride channel protein 2 isoform X3) c4(301142) c5(301143, 301144, 301145, 301146, 301147) c6( ) c7(iV, Cq, aPn, aPo, ha, aPp, aCs, aT, lr, ao, avl, HN, Ji, hg, eA, iD, cb, ji, akq, aR); #c1(CLCN3) c2(NP_001230301) c3(H(+)/CI(−) exchange transporter 3 isoform a) c4(86192) c5(86193, 86194, 86195, 86196, 86197, 86198, 86199, 86200, 86201, 86202, 86203, 86204, 86205, 86206, 86207, 86208, 86209, 86210, 86211, 86212, 86213, 86214, 86215, 86216) c6( ) c7(T, d, dh, aA, aq, eA, ae, B, ad, rY); #c1(CLCN3) c2(NP_001230303) c3(H(+)/CI(−) exchange transporter 3 isoform c) c4( ) c5(86217, 86218, 86219, 86220, 86221, 86222) c6( ) c7(T, d, dh, aA, aq, eA, a, B, ad, rY); #c1 (CLCN3) c2(NP_001820) c3(H(+)/CI(−) exchange transporter 3 isoform b) c4(121425, 121426, 121427, (21428, 121429) c5(121430, 121431, 121432, 121433, 121434, 121435) c6( ) c7(T, d, dh, aA, aq, eA, ae, B, ad, rY); #c1(CLCN3) c2(NP_776297) c3(H(+)/CI(−) exchange transporter 3 isoform e) c4(251073) c5(251074, 251075, 251076, 251077, 251078, 251079) c6( ) c7(T, d, dh, aA, aq, eA, ae, B, ad, rY); #c1(CLCN4) c2(NP_001243873) c3(H(+)/CI(−) exchange transporter 4 isoform 2) c4(91821) c5( ) c6( ) c7(bB, bE, ha, afH); #c1(CLCN4) c2(NP_001821) c3(H(+)/CI(−) exchange transporter 4 isoform 1) c4(121436, 121437, 121438) c5(121439, 121440, 121441, 121442, 121443, 121444, 121445, 121446, 121447, 121448, 121449, 121450, 121451, 121452, 121453, 121454, 121455, 121456, 121457, 121458, 121459, 121460, 121461, 121462) c6( ) c7(bB, bE, ha, afH); #c1 (CLCN5) c2(NP_001121370) c3(H(+)/CI(−) exchange transporter 5 isoform a) c4(45791, 45792, 45793) c5(45794, 45795, 45796, 45797, 45798, 45799, 45800, 45801, 45802, 45803) c6( ) c7(bL, aPr, Vp, aPt, TI, aAP, kQ, Rj, dK, cV, Ay, KW, n, aPs, dy, amC, aPq, wt, aY, amA, aAN); #c1 (CLCN5) c2(NP_001269092) c3(H(+)/CI(−) exchange transporter 5 isoform d) c4( ) c5(100408, 100409, 100410, 100411, 100412, 100413, 100414, 100415, 100416, 100417, 100418, 100419, 100420, 100421, 100422, 100423) c6( ) c7(bL, aPr, Vp, aPt, TI, aAP, KQ, Rj, dk, cV, Ay, KW, n, aPs, dy, amC, aPq, wt, aY, amA, aAN); #c1(CLCN6) c2(NP_001277) c3(chloride transport protein 6 isoform 1) c4( ) c5( ) c6( ) c7(bL, rY, cz); #c1(CLCN7) c2(NP_001107803) c3(H(+)/CI(−) exchange transporter 7 isoform b) c4(43007) c5(43008, 43009, 43010, 43011, 43012, 43013) c6( ) c7(zw, aPu, aPv, zs, Lq, nw, ajW, eA, bA); #c1(CLCN7) c2(NP_001278) c3(H(+)/CI(−) exchange transporter 7 isoform a) c4(111185, 111186) c5( ) c6( ) c7(zw, aPu, aPv, zs, Lq, nw, ajW, eA, bA); #c1(CLCN7) c2(XP_011520656) c3(H(+)/CI(−) exchange transporter 7 isoform X1) c4(315597) c5(315598, 315599, 315600, 315601, 315602) c6( ) c7(zw, aPu, aPv, zs, Lq, nw, ajW, eA, bA); #c1(CLCNKB) c2(XP_011538921) c3(chloride channel protein CIC-Kb isoform X1) c4(336989) c5( ) c6( ) c7(qa, Tl, ajT, XD, eR, mG, Rj, aPy, yj, cz, aPx, afV, Xy, adB, aPz, dy, Fg, aPw); #c1(CLCNKB) c2(XP_011538923) c3(chloride channel protein CIC-Kb isoform X3) c4( ) c5(336990, 336991, 336992, 336993, 336994, 336995) c6( ) c7(qa, Tl, ajT, XD, eR, mG, Rj, aPy, yj, cz, aPx, afV, Xy, adB, aPz, dy, Fg, aPw); #c1(CLDN1) c2(NP_066924) c3(claudin-1) c4(207501, 207502) c5(207503, 207504, 207505, 207506, 207507, 207508, 207509, 207510, 207511, 207512, 207513, 207514) c6( ) c7(T, aC, fR, aEj, ht, jx, aA, bB, n, ci, fu, q, aOF, ip, auO, dP, aE, gd, aT, aPA, aPB, aDE, qw, d, s, aW, ev, ff, r, fe, hN, lo, aq, aF, en, cl, hJ, t, g, bz, iV, iO, bE, vZ, AM, X, aqH, m, azr, in, ra, B); #c1(CLDN10) c2(NP_001153572) c3(claudin-10 isoform a_i1) c4(60657) c5(60658, 60659, 60660, 60661, 60662, 60663, 60664) c6( ) c7(n, cM, T, B, eZ); #c1(CLDN10) c2(NP_008915) c3(claudin-10 isoform b precursor) c4(169555) c5(169556, 169557, 169558, 169559, 169560, 169561) c6( ) c7(n, cM, T, B, eZ); #c1(CLDN10) c2(NP_878268) c3(claudin-10 isoform a) c4(257071) c5(257072, 257073, 257074, 257075, 257076, 257077) c6( ) c7(n, cM, T, B, eZ); #c1(CLDN11) c2(NP_005593) c3(claudin-Il isoform 1) c4(157751, 157752) c5(157753, 157754, 157755, 157756, 157757, 157758, 157759, 157760, 157761, 157762, 157763, 157764, 157765, 157766, 157767, 157768, 157769, 157770) c6( ) c7(T, Y, aA, aF, m, t, he, V, eW); #c1(CLDN14) c2(NP_036262) c3(claudin-14) c4(172676) c5(172677, 172678, 172679, 172680, 172681, 172682, 172683, 172684, 172685, 172686, 172687, 172688, 172689, 172690, 172691, 172692, 172693, 172694, 172695, 172696, 172697, 172698) c6( ) c7(aAN, alA, aPC); #c1(CLDN15) c2(NP_001172009) c3(claudin-15) c4(72215) c5(72216, 72217, 72218, 72219, 72220, 72221, 72222, 72223, 72224, 72225) c6( ) c7(arY); #c1(CLDN16) c2(NP_006571) c3(claudin-16) c4(166315, 166316) c5(166317, 166318, 166319, 166320, 166321, 166322, 166323, 166324, 166325, 166326, 166327, 166328) c6( ) c7(T, aPD, TI, eR, aW, aT, fP, cz, aPs, dy, m, Hb, t, aY); #c1(CLDN18) c2(NP_001002026) c3(claudin-18 isoform 2) c4(19370) c5(19371, 19372, 19373, 19374, 19375, 19376) c6( ) c7(uB, YL, T, av, Qy, aE, AO, eM, qt, iE, Yo, aA, kt, aF, ji, ci, g, iV, LV, bz, hX); #c1(CLDN18) c2(NP_057453) c3(claudin-18 isoform 1 precursor) c4(190961, 190962) c5(190963, 190964, 190965, 190966, 190967, 190968, 190969, 190970, 190971, 190972, 190973, 190974) c6( ) c7(uB, YL, T, av, Qy, aE, AO, eM, qt, iE, Yo, aA, kt, aF, ji, ci, g, iV, LV, bz, hX); #c1(CLDN2) c2(NP_001164566) c3(claudin-2) c4(67015) c5(67016, 67017, 67018, 67019, 67020, 67021, 67022, 67023, 67024, 67025, 67026, 67027, 67028, 67029) c6( ) c7(oO, T, lk, aA, bB, n, ci, jO, q, jF, l, u, aT, ji, s, aW, ff, r, fe, en, g, iV, ar, iO, eM, bE, gX, wz); #c1(CLDN23) c2(NP_919260) c3(claudin-23) c4(258326) c5(258327, 258328, 258329, 258330, 258331, 258332) c6( ) c7(mG, ac, aF, aA); #c1 (CLDN5) c2(NP_001124333) c3(claudin-5) c4(48705) c5(48706, 48707, 48708, 48709, 48710, 48711, 48712, 48713, 48714, 48715, 48716, 48717, 48718, 48719) c6( ) c7(ck, UH, agd, ac, c, aEj, aE, aW, yW, aw, ff, aFT, aT, cR, h, ci, m, en, dw, t, g); #c1 (CLDN6) c2(NP_067018) c3(claudin-6 precursor) c4(208396) c5(208397, 208398, 208399, 208400, 208401, 208402, 208403, 208404, 208405, 208406) c6( ) c7(jf, iu, T, Qy, eM, aW, Or, aT, aq, K, g, bz, B); #c1(CLDN7) c2(NP_001171952) c3 (claudin-7 isoform 2 precursor) c4(72098) c5(72099, 72100, 72101, 72102, 72103) c6( ) c7(uB, T, aC, Uc, aEj, al, jx, aA, bB, n, fr, ci, fu, AO, iJ, q, aOF, ad, ip, aE, gd, aT, aPB, ae, qw, avg, s, NE, qt, aW, ev, ff, r, hN, aq, aF, en, cl, hJ, t, g, bz, iV, iO, gk, eM, nB, bE, AM, X, m, azr, in, ra, B); #c1(CLDN8) c2(NP_955360) c3(claudin-8) c4(261538, 261539) c5(261540, 261541, 261542, 261543, 261544, 261545, 261546, 261547, 261548, 261549, 261550, 261551, 261552, 261553, 261554, 261555) c6( ) c7(Uc, fr, aE); #c1(CLDN9) c2(NP_066192) c3(claudin-9) c4(206842) c5(206843, 206844, 206845, 206846, 206847, 206848) c6( ) c7(ad); #c1(CLEC10A) c2(NP_006335) c3(C-type lectin domain family 10 member A isoform 2) c4(164/21) c5(164122, 164123, 164124, 164125, 164126, 164127, 164128, 164129, 164130, 164131) c6( ) c7(iu, T, fM, c, vT, yW, aW, Y, jh, aT, h, bK, ci, hp, j, y, Tl, aPE); #c1(CLEC10A) c2(NP_878910) c3(C-type lectin domain family 10 member A isoform 1) c4(257138) c5(257139, 257140, 257141, 257142, 257143, 257144) c6( ) c7(iu, T, fM, c, vT, yW, aW, Y, jh, aT, h, bK, ci, hp, j, y, TI, aPE); #c1 (CLEC11A) c2(NP_002966) c3(C-type lectin domain family 11 member A precursor) c4(134029, 134030) c5(134031, 134032, 134033, 134034, 134035, 134036, 134037, 134038, 134039, 134040, 134041, 134042, 134043, 134044, 134045, 134046) c6(134047, 134048, 134049, 134050) c7(bM, T, iV, ra, wu, oW, fe, qj, m, wm, aPF, t, fb, aPG, pC); #c1(CLEC12A) c2(NP_001193939) c3(C-type lectin domain family 12 member A isoform 3) c4(83250) c5(83251, 83252, 83253, 83254, 83255, 83256, 83257) c6( ) c7(T, fX, Of, jh, Og, y, bt); #c1(CLEC12A) c2(NP_001287659) c3(C-type lectin domain family 12 member A isoform 4) c4(112448, 112449) c5(112450, 112451, 112452, 112453, 112454, 112455, 112456, 112457, 112458, 112459, 112460, 112461, 112462, 112463, 112464, 112465) c6( ) c7(T, fX, Of, jh, Og, y, bt); #c1(CLEC12A) c2(NP_612210) c3(C-type lectin domain family 12 member A isoform 1) c4(236224) c5(236225, 236226, 236227, 236228, 236229) c6( ) c7(T, fX, Of, jh, Og, y, bt); #c1(CLEC12A) c2(NP_963917) c3(C-type lectin domain family 12 member A isoform 2) c4(262556, 262557) c5(262558, 262559, 262560, 262561, 262562, 262563) c6( ) c7(T, fX, Of, jh, Og, y, bt); #c1(CLEC12A) c2(XP_006719099) c3(C-type lectin domain family 12 member A isoform X7) c4( ) c5(292210, 292211) c6( ) c7(T, fx, Of, jh, Og, y, bt); #c1(CLEC14A) c2(NP_778230) c3(C-type lectin domain family 14 member A precursor) c4(251441, 251442) c5(251443, 251444, 251445, 251446, 251447, 251448, 251449, 251450, 251451, 251452, 251453, 251454) c6( ) c7(bL, mw, K, Ea, cd, cY); #c1(CLEC16A) c2(NP_001230332) c3(protein CLEC16A isoform 2) c4(86237) c5(86238, 86239, 86240, 86241, 86242, 86243) c6( ) c7(bL, eR, lo, yG, aw, hz, cl, aj, V, o, rw, aT, hK, bc, yF, A, lC, eZ, fe, hp, lY, OW, ac, y); #c1 (CLEC16A) c2(NP_056041) c3(protein CLEC16A isoform 1) c4(185259, 185260) c5(185261, 185262, 185263, 185264, 185265, 185266, 185267, 185268, 185269, 185270, 185271, 185272) c6( ) c7(bL, eR, lo, yG, aw, hz, cl, aj, V, o, rw, aT, hK, bc, yF, A, lC, eZ, fe, hp, lY, OW, ac, y); #c1(CLEC16A) c2(XP_011520736) c3(protein CLEC16A isoform X4) c4(315755) c5(315756, 315757, 315758, 315759, 315760, 315761) c6( ) c7(bL, eR, lo, yG, aw, hz, cl, aj, V, o, rw, aT, hK, bc, yF, A, lC, eZ, fe, hp, lY, OW, ac, y); #c1(CLEC1B) c2(NP_001092901) c3(C-type lectin domain family 1 member B isoform b) c4(39724, 39725) c5(39726, 39727, 39728, 39729, 39730, 39731, 39732, 39733, 39734, 39735, 39736, 39737, 39738, 39739, 39740, 39741) c6( ) c7(bL, T, A, cz, dX, j, y, sj); #c1(CLEC1B) c2(NP_057593) c3(C-type lectin domain family 1 member B isoform a) c4(191488) c5(191489, 191490, 191491, 191492, 191493, 191494) c6( ) c7(bL, T, A, cz, dX, j, y, sj); #c1(CLEC2A) c2(NP_001124183) c3(C-type lectin domain family 2 member A isoform 1) c4(48447) c5(48448, 48449, 48450, 48451, 48452, 48453) c6( ) c7(ai); #c1(CLEC2B) c2(NP_005118) c3(C-type lectin domain family 2 member B) c4(153046) c5(153047, 153048, 153049, 153050, 153051, 153052) c6( ) c7(aq); #c1(CLEC2D) c2(NP_001004419) c3(C-type lectin domain family 2 member D isoform 2) c4(20676) c5(20677, 20678, 20679, 20680, 20681, 20682) c6( ) c7(T, A, yE, cU, hK, eA, aDH); #c1(CLEC2D) c2(NP_001184246) c3(C-type lectin domain family 2 member D isoform 3) c4(75578) c5(75579, 75580, 75581, 75582, 75583, 75584) c6( ) c7(T, A, yE, cU, hK, eA, aDH); #c1(CLEC2D) c2(NP_001184247) c3(C-type lectin domain family 2 member D isoform 4) c4(75585) c5(75586, 75587, 75588, 75589, 75590, 75591) c6( ) c7(T, A, yE, cU, hK, eA, aDH); #c1(CLEC2D) c2(NP_001184248) c3(C-type lectin domain family 2 member D isoform 5) c4(75592) c5(75593, 75594, 75595, 75596, 75597, 75598) c6( ) c7(T, A, yE, cU, hK, eA, aDH); #c1(CLEC2D) c2(NP_037401) c3(C-type lectin domain family 2 member D isoform 1) c4(175302) c5(175303, 175304, 175305, 175306, 175307, 175308) c6( ) c7(T, A, yE, cU, hK, eA, aDH); #c1(CLEC2L) c2(NP_001073980) c3(C-type lectin domain family 2 member L) c4(36762) c5(36763, 36764, 36765, 36766, 36767, 36768) c6( ) c7(bL); #c1(CLEC3B) c2(NP_003269) c3(tetranectin precursor) c4(136952) c5(136953, 136954, 136955, 136956, (36957, 136958, 136959, 136960) c6(136961, 136962) c7(aPH, bE, r, bB, qN, g, q); #c1(CLEC4A) c2(NP_057268) c3(C-type lectin domain family 4 member A isoform 1) c4(189769) c5(189770, 189771, 189772, 189773, 189774, 189775) c6( ) c7(h, y, Ke); #c1(CLEC4A) c2(NP_919429) c3(C-type lectin domain family 4 member A isoform 3) c4(258593) c5(258594, 258595, 258596, 258597, 258598, 258599) c6( ) c7(h, y, Ke); #c1(CLEC4A) c2(NP_919430) c3(C-type lectin domain family 4 member A isoform 4) c4(258600) c5(258601, 258602, 258603, 258604, 258605, 258606) c6( ) c7(h, y, Ke); #c1(CLEC4A) c2(NP_919432) c3(C-type lectin domain family 4 member A isoform 2) c4(258621) c5(258622, 258623, 258624, 258625, 258626, 258627) c6( ) c7(h, y, Ke); #c1(CLEC4C) c2(NP_569708) c3(C-type lectin domain family 4 member C isoform 1) c4(233720) c5(233721, 233722, 233723, 233724, 233725, 233726, 233727, 233728, 233729, 233730) c6( ) c7(eZ); #c1(CLEC4C) c2(NP_987099) c3(C-type lectin domain family 4 member C isoform 2) c4(263406) c5(263407, 263408, 263409, 263410, 263411, 263412, 263413, 263414, 263415, 263416) c6( ) c7(eZ); #c1(CLEC4D) c2(NP_525126) c3(C-type lectin domain family 4 member D) c4(231203, 231204) c5(231205, 231206, 231207, 231208, 231209, 231210, 231211, 231212, 231213, 231214, 231215, 231216) c6( ) c7(ck, x, T, ady, fM, zn, azW, iU, iW, bt, cj, h, jh, ji, k, ho, Zb, aa, yR, aHE); #c1(CLEC4E) c2(NP_055173) c3 (C-type lectin domain family 4 member E) c4(179570) c5(179571, 179572, 179573, 179574, 179575, 179576) c6( ) c7(v, aa); #c1(CLEC4G) c2(NP_001231785) c3(C-type lectin domain family 4 member G isoform 2) c4(87522) c5(87523, 87524, 87525, 87526, 87527) c6( ) c7(wc); #c1(CLEC4G) c2(NP_940894) c3(C-type lectin domain family 4 member G isoform 1) c4(260063) c5(260064, 260065, 260066, 260067, 260068, 260069, 260070) c6( ) c7(wc); #c1(CLEC4M) c2(NP_001138376) c3(C-type lectin domain family 4 member M isoform 2) c4(56435) c5(56436, 56437, 56438, 56439, 56440, 56441) c6( ) c7(pA, fR, wc, jx, gC, h, j); #c1(CLEC4M) c2(NP_001138379) c3(C-type lectin domain family 4 member M isoform 11) c4(56442) c5(56443, 56444, 56445, 56446, 56447, 56448) c6( ) c7(pA, fR, wc, jx, gC, h, j); #c1 (CLEC4M) c2(NP_001138382) c3(C-type lectin domain family 4 member M isoform 8) c4(56449) c5(56450, 56451, 56452, 56453, 56454, 56455) c6( ) c7(pA, fR, wc, jx, gC, h, j); #c1(CLEC4M) c2(NP_001138383) c3(C-type lectin domain family 4 member M isoform 3) c4(56456) c5(56457, 56458, 56459, 56460, 56461, 56462) c6( ) c7(pA, fR, wc, jx, gC, h, j); #c1(CLEC4M) c2(NP_055072) c3(C-type lectin domain family 4 member M isoform 1) c4(178761, 178762) c5(178763, 178764, 178765, 178766, 178767, 178768, 178769, 178770, 178771, 178772, 178773, 178774) c6( ) c7(pA, fR, wc, jx, gC, h, j); #c1(CLEC4M) c2(XP_006722677) c3(C-type lectin domain family 4 member M isoform X4) c4(294813) c5(294814, 294815, 294816, 294817, 294818, 294819) c6( ) c7(pA, fR, wc, jx, gC, h, j); #c1(CLEC4M) c2(XP_006722678) c3(C-type lectin domain family 4 member M isoform X5) c4(294820) c5(294821, 294822, 294823, 294824, 294825, 294826) c6( ) c7(pA, fR, wc, jx, gC, h, j); #c1(CLEC5A) c2(NP_037384) c3(C-type lectin domain family 5 member A isoform 1) c4(175180) c5(175181, 175182, 175183, 175184, 175185, 175186) c6( ) c7(AH, Uq, aDm, pL, lf, ai, y); #c1(CLEC6A) c2(NP_001007034) c3 (C-type lectin domain family 6 member A) c4(22442) c5(22443, 22444, 22445, 22446, 22447, 22448) c6( ) c7(y, wR); #c1(CLEC7A) c2(NP_072092) c3(C-type lectin domain family 7 member A isoform b) c4(212670) c5(212671, 212672, 212673, 212674, 212675, 212676) c6( ) c7(gU, azW, aPJ, lH, aBl, azY, h, ji, aPl, aGP, Zf, cr, eZ, fe, iV, eE, azV, wR, CZ, m, lf, y); #c1(CLEC7A) c2(NP_922938) c3(C-type lectin domain family 7 member A isoform a) c4(258668) c5(258669, 258670, 258671, 258672, 258673, 258674, 258675, 258676, 258677, 258678, 258679) c6( ) c7(gU, azW, aPJ, lH, aBl, azY, h, ji, aPl, aGP, Zf, cr, eZ, fe, iV, eE, azV, wR, CZ, m, lf, y); #c1 (CLEC7A) c2(NP_922939) c3(C-type lectin domain family 7 member A isoform c) c4(258680) c5(258681, 258682, 258683, 258684, 258685, 258686) c6( ) c7(gU, azW, aPJ, lH, aBl, azY, h, ji, aPl, aGP, Zf, cr, eZ, fe, iV, eE, azV, wR, CZ, m, lf, y); #c1(CLEC7A) c2(NP_922940) c3(C-type lectin domain family 7 member A isoform d) c4(258687) c5(258688, 258689, 258690, 258691, 258692, 258693) c6( ) c7(gU, azW, aPJ, lH, aBl, azY, h, ji, aPl, aGP, Zf, cr, eZ, fe, iV, eE, azV, wR, CZ, m, lf, y); #c1(CLEC7A) c2(NP_922941) c3(C-type lectin domain family 7 member A isoform e) c4(258694) c5(258695, 258696, 258697, 258698, 258699, 258700) c6( ) c7(gU, azW, aPJ, lH, aBl, azY, h, ji, aPl, aGP, Zf, cr, eZ, fe, iV, eE, azV, wR, CZ, m, lf, y); #c1(CLEC7A) c2(NP_922945) c3(C-type lectin domain family 7 member A isoform f) c4(258701) c5(258702, 258703, 258704, 258705, 258706, 258707, 258708, 258709, 258710, 258711, 258712) c6( ) c7(gU, azW, aPJ, lH, aBl, azY, h, ji, aPl, aGP, Zf, cr, eZ, fe, iV, eE, azV, wR, CZ, m, lf, y); #c1(CLEC7A) c2(XP_006719198) c3(C-type lectin domain family 7 member A isoform X2) c4(292297) c5( ) c6( ) c7(gU, azW, aPJ, lH, aBl, azY, h, ji, aPl, aGP, Zf, cr, eZ, fe, iV, eE, azV, wR, CZ, m, lf, y); #c1(CLEC9A) c2(NP_997228) c3(C-type lectin domain family 9 member A) c4(265231) c5(265232, 265233, 265234, 265235, 265236, 265237) c6( ) c7(eh); #c1(CLECL1) c2(NP_001240679) c3(C-type lectin-like domain family 1 isoform 3) c4(88697) c5(88698, 88699, 88700, 88701, 88702, 88703) c6( ) c7(V); #c1(CLECL1) c2(NP_742001) c3(C-type lectin-like domain family 1 isoform 1) c4(247583) c5(247584, 247585, 247586, 247587, 247588, 247589) c6( ) c7(V); #c1(CLGN) c2(NP_004353) c3(calmegin precursor) c4(145589, 145590, 145591) c5(145592, 145593, 145594, 145595, 145596, 145597, 145598, 145599, 145600, 145601, 145602, 145603, 145604, 145605, 145606, 145607, 145608, 145609, 145610, 145611, 145612, 145613, 145614, 145615, 145616, 145617) c6( ) c7(np); #c1(CLIC1) c2(NP_001279) c3(chloride intracellular channel protein 1) c4(11/202) c5( ) c6( ) c7(Ye, T, A, JI, eZ, aA, aT, io, h, aF, eA, lV, it, X); #c1(CLIC3) c2(NP_004660) c3(chloride intracellular channel protein 3) c4(148597, 148598) c5(148599, 148600, 148601, 148602, 148603, 148604, 148605, 148606, 148607, 148608, 148609, 148610) c6( ) c7(el, dl, eb); #c1(CLIC5) c2(NP_001107558) c3(chloride intracellular channel protein 5 isoform a) c4(42829) c5(42830, 42831, 42832, 42833, 42834, 42835) c6( ) c7(vo); #c1(CLIC5) c2(NP_001242952) c3(chloride intracellular channel protein 5 isoform c) c4(89425) c5(89426, 89427, 89428, 89429, 89430, 89431) c6( ) c7(vo); #c1(CLIC5) c2(NP_058625) c3(chloride intracellular channel protein 5 isoform b) c4(192826) c5(192827, 192828, 192829, 192830, 192831, 192832) c6( ) c7(va); #c1 (CLIC6) c2(NP_444507) c3(chloride intracellular channel protein 6) c4(230334) c5(230335, 230336, 230337, 230338, 230339, 230340) c6( ) c7(aPK); #c1(CLINT1) c2(NP_001182484) c3(clathrin interactor 1 isoform 1) c4(74961) c5(74962, 74963, 74964, 74965, 74966, 74967, 74968, 74969, 74970, 74971, 74972, 74973, 74974, 74975, 74976, 74977, 74978, 74979, 74980, 74981) c6( ) c7(gl, he, bA); #c1(CLINT1) c2(NP_001182485) c3(clathrin interactor 1 isoform 3) c4(74982) c5( ) c6( ) c7(gl, he, bA); #c1(CLINT1) c2(NP_055481) c3(clathrin interactor 1 isoform 2) c4(181370) c5(181371, 181372, 181373, 181374, 181375, 181376) c6( ) c7(gl, he, bA); #c1(CLIP1) c2(NP_001234926) c3(CAP-Gly domain-containing linker protein 1 isoform c) c4(87782, 87783) c5(87784, 87785, 87786, 87787, 87788, 87789, 87790, 87791, 87792, 87793, 87794, 87795, 87796, 87797, 87798, 87799, 87800, 87801) c6( ) c7(bL, A, alC, c, jx, eX, aT, eV, aj, dG, t, J, bN, hX); #c1(CLIP1) c2(NP_002947) c3(CAP-Gly domain-containing linker protein 1 isoform a) c4(133825, 133826) c5(133827, 133828, 133829, 133830, 133831, 133832, 133833, 133834, 133835, 133836) c6( ) c7(bL, A, alC, c, jx, eX, aT, eV, aj, dG, t, J, bN, hX); #c1 (CLIP1) c2(NP_937883) c3(CAP-Gly domain-containing linker protein 1 isoform b) c4(259402, 259403) c5(259404, 259405, 259406, 259407, 259408, 259409, 259410, 259411, 259412, 259413, 259414, 259415, 259416, 259417, 259418, 259419, 259420, 259421, 259422) c6( ) c7(bL, A, alC, c, jx, eX, aT, eV, aj, dG, t, J, bN, hX); #c1(CLIP1) c2(XP_005253650) c3(CAP-Gly domain-containing linker protein 1 isoform X1) c4(272606, 272607) c5(272608, 272609, 272610, 272611, 272612, 272613) c6( ) c7(bL, A, alC, c, jx, eX, aT, eV, aj, dG, t, J, bN, hX); #c1(CLIP1) c2(XP_006719615) c3(CAP-Gly domain-containing linker protein 1 isoform X2) c4(292706) c5(292707, 292708, 292709, 292710, 292711) c6( ) c7(bL, A, alC, c, jx, eX, aT, eV, aj, dG, t, J, bN, hX); #c1(CLIP2) c2(NP_003379) c3(CAP-Gly domain-containing linker protein 2 isoform 1) c4(138037, 138038) c5(138039, 138040, 138041, 138042, 138043, 138044, 138045, 138046, 138047, 138048, 138049, 138050, 138051, 138052, 138053, 138054, 138055, 138056, 138057, 138058) c6( ) c7(bL, OV, alC, A, c, jx, aj, dG, J, bN, hX); #c1(CLIP2) c2(NP_115797) c3(CAP-Gly domain-containing linker protein 2 isoform 2) c4(223936, 223937) c5(223938, 223939, 223940, 223941, 223942, 223943, 223944, 223945, 223946, 223947, 223948, 223949) c6( ) c7(bL, OV, alC, A, c, jx, aj, dG, J, bN, hX); #c1(CLK1) c2(NP_001155879) c3(dual specificity protein kinase CLK1 isoform 2) c4(61617, 61618) c5(61619, 61620, 61621, 61622, 61623, 61624) c6( ) c7(ds, asp, U); #c1(CLK1) c2(NP_004062) c3(dual specificity protein kinase CLK1 isoform 1) c4(143230) c5(143231, 143232, 143233, 143234, 143235, 143236, 143237, 143238, 143239, 143240, 143241, 143242, 143243) c6( ) c7(ds, asp, U); #c1(CLK2) c2(NP_001281267) c3(dual specificity protein kinase CLK2 isoform 1) c4(111641, 111642) c5( ) c6( ) c7(it, aF, nA, aA); #c1(CLK2) c2(NP_001281268) c3(dual specificity protein kinase CLK2 isoform 3) c4( ) c5(111643, 111644, 111645, 111646, 111647) c6( ) c7(it, aF, nA, aA); #c1(CLK2) c2(NP_003984) c3(dual specificity protein kinase CLK2 isoform 2) c4(142644) c5( ) c6( ) c7(it, aF, nA, aA); #c1(CLK2) c2(XP_005276801) c3(dual specificity protein kinase CLK2 isoform X2) c4(285721) c5(285722, 285723, 285724, 285725, 285726, 285727, 285728, 285729, 285730, 285731, 285732, 285733, 285734) c6( ) c7(it, aF, nA, aA); #c1(CLK2) c2(XP_011545227) c3(dual specificity protein kinase CLK2 isoform X1) c4( ) c5(344009, 344010, 344011, 344012, 344013, 344014, 344015, 344016, 344017, 344018) c6( ) c7(it, aF, nA, aA); #c1(CLLU1) c2(NP_001020404) c3(chronic lymphocytic leukemia up-regulated protein 1) c4(28732) c5(28733, 28734, 28735, 28736, 28737, 28738) c6( ) c7(ad, cj); #c1(CLMN) c2(NP_079010) c3(calmin) c4(216538) c5(216539, 216540, 216541, 216542, 216543, 216544) c6( ) c7(cm, j); #c1(CLMP) c2(NP_079045) c3(CXADR-like membrane protein precursor) c4(216737) c5(216738, 216739, 216740, 216741, 216742, 216743) c6( ) c7(j, aPL, bk, cj); #c1(CLN3) c2(NP_000077) c3(battenin isoform a) c4(1234, 1235, 1236, 1237) c5(1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270) c6( ) c7(aPO, T, aA, bB, ci, aR, Sw, mP, hb, aT, aiW, K, aPN, d, lR, aW, aPP, cm, nC, rY, bE, U, Lp, Lq, nw, aPM, X, de); #c1(CLN3) c2(NP_001273033) c3 (battenin isoform b) c4(104201) c5(104202, 104203, 104204, 104205, 104206, 104207) c6( ) c7(aPO, T, aA, bB, ci, aR, Sw, mP, hb, aT, aiW, K, aPN, d, lR, aW, aPP, cm, nC, rY, bE, U, Lp, Lq, nw, aPM, X, de); #c1(CLN3) c2(NP_001273034) c3(battenin isoform c) c4(104208, 104209) c5(104210, 104211, 104212, 104213, 104214, 104215, 104216, 104217, 104218, 104219, 104220, 104221, 104222) c6( ) c7(aPO, T, aA, bB, ci, aR, Sw, mP, hb, aT, aiW, K, aPN, d, lR, aW, aPP, cm, nC, rY, bE, U, Lp, Lq, nw, aPM, X, de); #c1(CLN3) c2(NP_001273038) c3(battenin isoform d) c4(104242) c5(104243, 104244, 104245, 104246, 104247) c6( ) c7(aPO, T, aA, bB, ci, aR, Sw, mP, hb, aT, aiW, K, aPN, d, lR, aW, aPP, cm, nC, rY, bE, U, Lp, Lq, nw, aPM, X, de); #c1(CLN3) c2(NP_001273039) c3(battenin isoform e) c4(104248) c5(104249, 104250, 104251, 104252, 104253) c6( ) c7(aPO, T, aA, bB, ci, aR, Sw, mP, hb, aT, aiW, K, aPN, d, lR, aW, aPP, cm, nC, rY, bE, U, Lp, Lq, nw, aPM, X, de); #c1(CLN5) c2(NP_006484) c3(ceroid-lipofuscinosis neuronal protein 5) c4(165597, 165598, 165599) c5(165600, 165601, 165602, 165603, 165604, 165605, 165606, 165607, 165608, 165609, 165610, 165611, 165612, 165613, 165614, 165615, 165616, 165617) c6( ) c7(T, apM, aPQ, eM, aPR, hb, aW, ha, aPS, aT, U, Lq, aPT, ci, nA, aR, rY, alX); #c1(CLN6) c2(NP_060352) c3(ceroid-lipofuscinosis neuronal protein 6) c4(195543, 195544, 195545) c5(195546, 195547, 195548, 195549, 195550, 195551, 195552, 195553, 195554, 195555, 195556, 195557, 195558, 195559, 195560, 195561, 195562, 195563) c6( ) c7(aPQ, aPU, U, yZ, Lq, K, aPT, yU, rY); #c1(CLN8) c2(NP_061764) c3(protein CLN8) c4(200454) c5(200455, 200456, 200457, 200458, 200459, 200460) c6( ) c7(vU, aPV, ha, HN, aPS, nA, rY); #c1(CLNK) c2(NP_443196) c3(cytokine-dependent hematopoietic cell linker) c4( ) c5(229795, 229796, 229797, 229798, 229799, 229800, 229801, 229802, 229803, 229804, 229805, 229806, 229807, 229808, 229809, 229810, 229811, 229812, 229813, 229814) c6( ) c7(gk, c, al, EQ, qj); #c1(CLNK) c2(XP_011512076) c3(cytokine-dependent hematopoietic cell linker isoform X1) c4(302721) c5( ) c6( ) c7(gk, c, al, EQ, qj); #c1(CLNK) c2(XP_011512077) c3(cytokine-dependent hematopoietic cell linker isoform X2) c4(302722, 302723) c5(302724, 302725) c6( ) c7(gk, c, al, EQ, qj); #c1 (CLNS1A) c2(NP_001284) c3(methylosome subunit pICln) c4(112396) c5( ) c6( ) c7(Y); #c1(CLOCK) c2(NP_004889) c3(circadian locomoter output cycles protein kaput) c4(150578) c5(150579, 150580, 150581, 150582, 150583, 150584, 150585, 150586, 150587, 150588, 150589, 150590, 150591, 150592, 150593) c6( ) c7(bM, T, ea, e, bO, gf, iX, ta, hu, aW, fc, cm, mZ, iA, cj, uJ, dD, gU, alc, n, aj, fr, ct, Nv, mY, mV, he, mU, Jj, aPZ, fe, cM, alb, mX, hq, cA, hk, mg, vL, aPX, jd, mT, bL, jx, Nt, cz, mS, ti, v, q, J, eA, aPW, MR, hb, aql, aT, bK, ae, af, il, nO, Y, r, en, ik, bN, aPY, pH, Dy, and, jh, Gd, bS, eq, Fc, ad, dP, oz, mW, sO, Os, ala, na, d, aaB, ff, dq, Z, es, bE, ei, y, aqD); #c1 (CLP1) c2(NP_001136069) c3(polyribonucleotide 5′-hydroxyl-kinase Clp1 isoform 2) c4(54253) c5(54254, 54255, 54256, 54257, 54258, 54259, 54260, 54261, 54262, 54263, 54264, 54265, 54266, 54267, 54268, 54269, 54270, 54271) c6( ) c7(Ae, QK, jW, oj, Ak); #c1(CLP1) c2(NP_006822) c3 (polyribonucleotide 5′-hydroxyl-kinase Clp1 isoform 1) c4(168407, 168408, 168409, 168410) c5(168411, 168412, 168413, 168414, 168415, 168416, 168417, 168418, 168419, 168420, 168421, 168422, 168423, 168424, 168425, 168426) c6( ) c7(Ae, QK, jW, aj, Ak); #c1(CLPP) c2(NP_006003) c3(ATP-dependent Clp protease proteolytic subunit, mitochondrial precursor) c4(161/16) c5(161117, 161118, 161119, 16/120, 161121, 161122) c6( ) c7(K, Bh, alf, alg, ale); #c1(CLPS) c2(NP_001239527) c3(colipase isoform 3 preproprotein) c4(88470) c5(88471, 88472, 88473, 88474, 88475, 88476) c6( ) c7(bL, aj, v, K, bq, J); #c1(CLPS) c2(NP_001823) c3(colipase isoform 1 preproprotein) c4(121504) c5(121505, 121506, 121507, 121508, 121509, 121510) c6( ) c7(bL, aj, v, K, bq, J); #c1(CLPTM1) c2(NP_001269104) c3(cleft lip and palate transmembrane protein 1 isoform 3) c4(100443) c5(100444, 100445, 100446, 100447, 100448, 100449) c6( ) c7(Na, T, apM, eM, Y, Nc, bz, ad); #c1 (CLPTM1) c2(NP_001269105) c3(cleft lip and palate transmembrane protein 1 isoform 4) c4( ) c5(100450, 100451, 100452, 100453, 100454) c6( ) c7(Na, T, apM, eM, Y, Nc, bz, ad); #c1(CLPTM1) c2(NP_001285) c3(cleft lip and palate transmembrane protein 1 isoform 2) c4(112397, 112398, 112399) c5(112400, 112401, 112402, 112403, 112404, 112405, 112406, 112407, 112408, 112409, 112410, 112411, 112412) c6( ) c7(Na, T, apM, eM, Y, Nc, bz, ad); #c1(CLPTM1L) c2(NP_110409) c3(cleft lip and palate transmembrane protein 1-like protein) c4(220120, 220121) c5(220122, 220123, 220124, 220125, 220126, 220127, 220128, 220129, 220130, 220131, 220132, 220133) c6( ) c7(T, aC, yk, if, NT, bB, aj, hy, j, qN, q, ad, eA, av, aE, amJ, afQ, aT, hn, dL, af, vT, aW, Y, ff, r, kp, en, bu, g, t, ik, bz, sS, eM, bE, hk, Lb, m, it, iu); #c1 (CLRN1) c2(NP_001182723) c3(clarin-1 isoform d) c4(75154) c5(75155, 75156, 75157, 75158, 75159, 75160) c6( ) c7(mG, ali, alj, oc, nw, nk, nC, alh); #c1(CLRN1) c2(NP_001243748) c3(clarin-1 isoform e) c4( ) c5(91640) c6( ) c7(mG, ali, alj, oc, nw, nk, nC, alh); #c1 (CLRN1) c2(NP_443721) c3(clarin-1 isoform c) c4(230004, 230005) c5(230006, 230007, 230008, 230009, 230010, 230011, 230012, 230013, 230014, 230015, 230016) c6( ) c7(mG, ali, alj, oc, nw, nk, nC, alh); #c1(CLRN1) c2(NP_777367) c3(clarin-1 isoform a) c4(251108) c5(251109, 251110, 251111, 251112, 251113, 251114) c6( ) c7(mG, ali, alj, oc, nw, nk, nC, alh); #c1(CLSPN) c2(XP_011540241) c3(claspin isoform X2) c4(338641) c5(338642, 338643, 338644, 338645, 338646, 338647) c6( ) c7(T, aW, if, aT, bK, ci, K, m, bC, qN); #c1(CLSPN) c2(XP_011540244) c3(claspin isoform X5) c4(338648) c5(338649, 338650, 338651, 338652, 338653, 338654) c6( ) c7(T, aW, if, aT, bk, ci, K, m, bC, qN); #c1(CLSTN1) c2(NP_001289812) c3(calsyntenin-1 isoform 3 precursor) c4(114637, 114638) c5(114639, 114640, 114641, 114642, 114643, 114644, 114645, 114646, 114647, 114648, 114649, 114650, 114651, 114652, 114653, 114654, 114655, 114656, 114657, 114658, 114659, 114660) c6(114661, 114662, 114663, 114664, 114665, 114666) c7(T, d, oV, zb, r, fd, q, cm, ae, B, ad, De); #c1(CLSTN2) c2(NP_071414) c3 (calsyntenin-2 precursor) c4(211544) c5(211545, 211546, 211547, 211548, 211549, 211550) c6( ) c7(fe, aq, FW, ot); #c1(CLTA) c2(NP_001070145) c3(clathrin light chain A isoform c) c4(34587) c5(34588, 34589, 34590, 34591, 34592, 34593) c6( ) c7(nO, eZ, jh, uu, ci, y, nC, Nh); #c1(CLTA) c2(NP_001171689) c3(clathrin light chain A isoform d) c4(7/296) c5(71297, 71298, 71299, 71300, 71301, 71302, 71303, 71304, 71305, 71306) c6( ) c7(nO, eZ, jh, uu, ci, y, nC, Nh); #c1(CLTA) c2(NP_001171691) c3(clathrin light chain A isoform f) c4(71307) c5(71308, 71309, 71310, 71311, 71312, 71313) c6( ) c7(nO, eZ, jh, uu, ci, y, nC, Nh); #c1(CLTA) c2(NP_001824) c3(clathrin light chain A isoform a) c4(121511) c5(121512, 121513, 121514, 121515, 121516, 121517) c6( ) c7(nO, eZ, jh, uu, ci, y, nC, Nh); #c1(CLTA) c2(NP_009027) c3(clathrin light chain A isoform b) c4(170296) c5(170297, 170298, 170299, 170300, 170301, 170302, 170303, 170304, 170305, 170306) c6( ) c7(nO, eZ, jh, uu, ci, y, nC, Nh); #c1(CLTC) c2(NP_004850) c3(clathrin heavy chain 1 isoform 1) c4(150303) c5(150304, 150305, 150306, 150307, 150308, 150309) c6( ) c7(bv, T, agd, F, fM, d, aE, iA, dc, aW, HM, RX, jh, aT, h, fr, eA, ae, B, ad); #c1 (CLTCL1) c2(NP_001826) c3(clathrin heavy chain 2 isoform 2) c4(121518) c5(121519, 121520, 121521, 121522, 121523, 121524) c6( ) c7(bv, ip); #c1(CLTCL1) c2(NP_009029) c3(clathrin heavy chain 2 isoform 1) c4(170307) c5(170308, 170309, 170310, 170311, 170312, 170313, 170314, 170315, 170316, 170317) c6( ) c7(bv, ip); #c1(CLU) c2(NP_001822) c3(clusterin preproprotein) c4(121463, 121464, 121465) c5(121466, 121467, 121468, 121469, 121470, 121471, 121472, 121473, 121474, 121475, 121476, 121477, 121478, 121479, 121480, 121481, 121482, 121483, 121484, 121485, 121486, 121487, 121488, 121489, 121490, 121491, 121492, 121493, 121494, 121495, 121496, 121497, 121498, 121499, 121500, 121501, 121502, 121503) c6( ) c7(gh, G, T, fV, ea, e, al, if, bB, lN, ci, aR, aE, dV, K, wt, oi, H, MO, eL, cp, li, eG, dH, aW, aNb, hl, zC, g, as, hJ, eF, uC, cm, eY, ba, y, eM, AM, U, nw, Hb, m, R, iu, dt, dD, n, aj, ww, fr, qN, PJ, fK, cd, Mo, amB, ql, eV, dL, ia, aq, dw, cM, avN, W, vD, dB, hk, hL, E, uz, sa, it, B, L, bL, dA, DD, dk, eX, aA, cz, j, q, J, eA, RI, lg, aT, kf, cy, bK, bq, an, aFm, ae, af, eu, CB, Lu, alk, s, oV, Y, Lf, bg, r, TQ, en, bu, cl, t, nC, ik, Qi, kP, pZ, ll, cS, np, dy, LS, VB, iW, aaQ, ha, jh, iD, oH, ah, eq, ad, aoH, Jm, fU, av, sO, abP, d, cH, ff, eH, eZ, aF, iV, bz, bF, D, Hw, gk, zr, bE, ME, lC, X, EB); #c1(CLU) c2(XP_006716347) c3(clusterin isoform X1) c4(290228, 290229, 290230, 290231) c5(290232, 290233, 290234, 290235, 290236, 290237, 290238, 290239, 290240, 290241, 290242, 290243, 290244, 290245, 290246, 290247, 290248, 290249, 290250, 290251, 290252, 290253, 290254, 290255, 290256, 290257, 290258, 290259, 290260, 290261) c6( ) c7(gh, G, T, fV, ea, e, al, if, bB, lN, ci, aR, aE, dV, K, wt, oi, H, MO, eL, cp, li, eG, dH, aW, aNb, hl, zC, g, as, hJ, eF, uC, cm, eY, ba, y, eM, AM, U, nw, Hb, m, R, iu, dt, dD, n, aj, ww, fr, qN, PJ, fK, cd, Mo, amB, ql, eV, dL, ia, aq, dw, cM, avN, W, vD, dB, hk, hL, E, uz, sa, it, B, L, bL, dA, DD, dk, eX, aA, cz, j, q, J, eA, RI, lg, aT, kf, cy, bK, bq, an, aFm, ae, af, eu, CB, Lu, alk, s, oV, Y, Lf, bg, r, TQ, en, bu, cl, t, nC, ik, Qi, kP, pZ, ll, cS, np, dy, LS, VB, iW, aal, ha, jh, iD, oH, ah, eq, ad, aoH, Jm, fU, av, sO, abP, d, cH, ff, eH, eZ, aF, iV, bz, bF, D, Hw, gk, zr, bE, ME, lC, X, EB); #c1(CLUAP1) c2(NP_055856) c3(clusterin-associated protein 1 isoform 1) c4(183942) c5(183943, 183944, 183945, 183946, 183947, 183948, 183949, 183950, 183951, 183952, 183953) c6( ) c7(av, T, bE, bB, eF, X); #c1(CLUAP1) c2(NP_079069) c3(clusterin-associated protein 1 isoform 2) c4( ) c5(216883, 216884, 216885, 216886, 216887, 216888, 216889, 216890, 216891, 216892, 216893) c6( ) c7(av, T, bE, bB, eF, X); #c1(CLUAP1) c2(XP_006720930) c3(clusterin-associated protein 1 isoform X1) c4(293791, 293792) c5( ) c6( ) c7(av, T, bE, bB, eF, X); #c1(CLUL1) c2(NP_001275965) c3(clusterin-like protein 1 isoform a) c4(107547) c5(107548, 107549, 107550, 107551, 107552, 107553, 107554, 107555, 107556, 107557, 107558, 107559, 107560, 107561, 107562, 107563, 107564, 107565, 107566, 107567, 107568) c6(107569, 107570, 107571, 107572, 107573, 107574) c7(aT); #c1(CLUL1) c2(NP_055225) c3(clusterin-like protein 1 isoform b precursor) c4(179937) c5( ) c6( ) c7(aT); #c1(CLUL1) c2(XP_005258160) c3(clusterin-like protein 1 isoform X3) c4( ) c5(275535) c6( ) c7(aT); #c1(CLVS1) c2(NP_775790) c3(clavesin-1) c4(250317) c5(250318, 250319, 250320, 250321, 250322, 250323, 250324, 250325, 250326, 250327, 250328, 250329, 250330, 250331, 250332, 250333) c6( ) c7(B, af); #c1(CLVS1) c2(XP_011515773) c3(clavesin-1 isoform X2) c4(308575) c5( ) c6( ) c7(B, af); #c1(CLYBL) c2(NP_996531) c3(citrate lyase subunit beta-like protein, mitochondrial precursor) c4(264041, 264042) c5(264043, 264044, 264045, 264046, 264047, 264048, 264049, 264050, 264051, 264052, 264053, 264054, 264055, 264056, 264057, 264058) c6( ) c7(h, qs, bz, fh); #c1(CLYBL) c2(XP_005254088) c3(citrate lyase subunit beta-like protein, mitochondrial isoform X2) c4(272882, 272883, 272884, 272885, 272886) c5(272887, 272888, 272889, 272890, 272891, 272892, 272893, 272894, 272895, 272896, 272897, 272898, 272899, 272900, 272901, 272902, 272903, 272904, 272905, 272906, 272907, 272908, 272909, 272910, 272911, 272912, 272913, 272914, 272915, 272916, 272917, 272918, 272919, 272920) c6( ) c7(h, qs, bz, fh); #c1(CMA1) c2(NP_001827) c3(chymase preproprotein) c4(121525, 121526) c5(121527, 121528, 121529, 121530, 121531, 121532, 121533) c6( ) c7(bL, bM, T, dD, fM, fG, aw, jh, aA, cz, fd, j, ci, eq, pM, eA, ad, mj, cc, aU, apJ, aT, DO, vc, vB, jy, K, hg, bq, fb, ae, eu, hm, ul, uX, eG, d, aW, vj, eH, Ce, fe, aF, aB, aiz, dw, eE, eF, bz, cm, ac, aQm, z, bo, vy, uo, gZ, dy, bD, lC, vQ); #c1(CMAS) c2(NP_061156) c3(N-acylneuraminate cytidylyltransferase) c4(199638) c5(199639, 199640, 199641, 199642, 199643, 199644) c6( ) c7(T, d, py, oV, fc, cP, anx, fb, q, hv); #c1(CMC1) c2(NP_872329) c3(COX assembly mitochondrial protein homolog) c4(256132) c5(256133, 256134, 256135, 256136, 256137) c6( ) c7(Ck, Dg, aln, aFy, Dj, fq); #c1(CMC2) c2(NP_064573) c3(COX assembly mitochondrial protein 2 homolog) c4(203041) c5(203042, 203043, 203044, 203045, 203046, 203047) c6( ) c7(dV, aT); #c1(CMC4) c2(NP_001018024) c3(cx9C motif-containing protein 4) c4(26873) c5(26874, 26875, 26876, 26877, 26878, 26879, 26880, 26881, 26882, 26883, 26884, 26885, 26886) c6( ) c7(ck, fM, Kk, qh, kH, atf, bC, bd, ho); #c1(CMIP) c2(NP_085132) c3(C-Maf-inducing protein isoform Tc-Mip) c4(219359, 219360, 219361) c5(219362, 219363, 219364, 219365, 219366, 219367, 219368, 219369, 219370, 219371, 219372, 219373) c6( ) c7(bL, Y, aw, VT, ah, yA, aha, Kq); #c1(CMIP) c2(NP_938204) c3(C-Maf-inducing protein isoform C-Mip) c4(259783) c5(259784, 259785, 259786, 259787, 259788, 259789) c6( ) c7(bL, Y, aw, VT, ah, yA, aha, Kq); #c1(CMIP) c2(XP_005256236) c3(C-Maf-inducing protein isoform X2) c4( ) c5(274013) c6( ) c7(bL, Y, aw, VT, ah, yA, aha, Kq); #c1(CMIP) c2(XP_011521655) c3(C-Maf-inducing protein isoform X3) c4( ) c5(316431, 316432, 316433, 316434, 316435) c6( ) c7(bL, Y, aw, VT, ah, yA, aha, Kq); #c1(CMKLR1) c2(NP_001135815) c3(chemokine-like receptor 1 isoform a) c4(53310, 53311, 53312, 53313) c5(53314, 53315, 53316, 53317, 53318, 53319, 53320, 53321, 53322, 53323, 53324, 53325, 53326, 53327, 53328, 53329, 53330, 53331, 53332, 53333, 53334, 53335, 53336, 53337, 53338, 53339, 53340) c6( ) c7(cZ, eM, aw, fc, cr, Vv, v, dd, y, alo); #c1 (CMKLR1) c2(NP_004063) c3(chemokine-like receptor 1 isoform b) c4( ) c5(143244, 143245, 143246, 143247, 143248, 143249, 143250, 143251) c6( ) c7(cZ, eM, aw, fc, cr, Vv, v, dd, y, alo); #c1(CMPK2) c2(NP_001243406) c3(UMP-CMP kinase 2, mitochondrial isoform 2 precursor) c4( ) c5(90700, 90701, 90702, 90703, 90704, 90705) c6( ) c7(ck, x, T, hd, gU, aE, iU, afm, aW, if, hl, aT, h, hn, K, k, qN, eA, dr, MG); #c1(CMPK2) c2(NP_001243407) c3(UMP-CMP kinase 2, mitochondrial isoform 3 precursor) c4(90706) c5(90707, 90708, 90709, 90710, 90711, 90712) c6( ) c7(ck, x, T, hd, gU, aE, iU, afm, aW, if, hl, aT, h, hn, K, k, qN, eA, dr, MG); #c1(CMPK2) c2(NP_997198) c3(UMP-CMP kinase 2, mitochondrial isoform 1 precursor) c4(265158, 265159) c5(265160, 265161, 265162, 265163, 265164, 265165) c6( ) c7(ck, x, T, hd, gU, aE, iU, afm, aW, if, hl, aT, h, hn, K, k, qN, eA, dr, MG); #c1(CMSS1) c2(NP_001161396) c3 (protein CMSS1 isoform 2) c4(65693) c5(65694, 65695, 65696, 65697, 65698, 65699) c6( ) c7(EQ); #c1(CMSS1) c2(NP_115735) c3(protein CMSS1 isoform 1) c4(223745) c5(223746, 223747, 223748, 223749, 223750, 223751) c6( ) c7(EQ); #c1(CMTM3) c2(NP_853531) c3(CKLF-like MARVEL transmembrane domain-containing protein 3) c4(255155, 255156) c5(255157, 255158, 255159, 255160, 255161, 255162, 255163, 255164, 255165, 255166, 255167, 255168, 255169, 255170) c6( ) c7(Lf, aF, ci, bu, aA); #c1(CMTM3) c2(XP_011521163) c3(CKLF-like MARVEL transmembrane domain-containing protein 3 isoform X2) c4( ) c5(316109, 316110, 316111, 316112, 316113, 316114) c6( ) c7(Lf, aF, ci, bu, aA); #c1(CMTM5) c2(NP_001032365) c3(CKLF-like MARVEL transmembrane domain-containing protein 5 isoform c) c4(31273) c5(31274, 31275, 31276, 31277, 31278, 31279) c6( ) c7(ck, T, ci, iT); #c1(CMTM5) c2(NP_001275673) c3(CKLF-like MARVEL transmembrane domain-containing protein 5 isoform d) c4(107018) c5(107019, 107020, 107021, 107022, 107023, 107024) c6( ) c7(ck, T, ci, iT); #c1(CMTM5) c2(NP_001275674) c3(CKLF-like MARVEL transmembrane domain-containing protein 5 isoform e) c4(107025) c5(107026, 107027, 107028, 107029, 107030, 107031) c6( ) c7(ck, T, ci, iT); #c1(CMTM5) c2(NP_001275675) c3(CKLF-like MARVEL transmembrane domain-containing protein 5 isoform f) c4(107032) c5(107033, 107034, 107035, 107036, 107037, 107038) c6( ) c7(ck, T, ci, iT); #c1(CMTM5) c2(NP_612469) c3(CKLF-like MARVEL transmembrane domain-containing protein 5 isoform a) c4(236614) c5(236615, 236616, 236617, 236618, 236619, 236620) c6( ) c7(ck, T, ci, lT); #c1(CMTM7) c2(NP_612419) c3(CKLF-like MARVEL transmembrane domain-containing protein 7 isoform a) c4(236421) c5(236422, 236423, 236424, 236425, 236426, 236427) c6( ) c7(if, cd, ci); #c1(CMTM7) c2(NP_852137) c3(CKLF-like MARVEL transmembrane domain-containing protein 7 isoform b) c4(254917) c5(254918, 254919, 254920, 254921, 254922, 254923) c6( ) c7(if, cd, ci); #c1(CMTM8) c2(NP_849199) c3(CKLF-like MARVEL transmembrane domain-containing protein 8) c4(254513) c5(254514, 254515, 254516, 254517, 254518, 254519) c6( ) c7(fq, bK, ac, pN); #c1(CMTR1) c2(NP_055865) c3(cap-specific mRNA (nucleoside-2′-0-)-methyltransferase 1) c4(184018) c5(184019, 184020, 184021, 184022, 184023, 184024) c6( ) c7(bv, aW, aw, g, aT); #c1(CMTR1) c2(XP_005249012) c3(cap-specific mRNA (nucleoside-2′-0-)-methyltransferase 1 isoform X1) c4(268850, 268851) c5(268852, 268853, 268854, 268855, 268856, 268857, 268858, 268859, 268860, 268861, 268862, 268863) c6( ) c7(bv, aW, aw, g, aT); #c1(CMTR1) c2(XP_006715091) c3(cap-specific mRNA (nucleoside-2′-0-)-methyltransferase 1 isoform X2) c4(288627, 288628) c5(288629, 288630, 288631, 288632, 288633, 288634, 288635, 288636, 288637, 288638, 288639, 288640, 288641, 288642, 288643, 288644) c6( ) c7(bv, aW, aw, g, aT); #c1(CMTR2) c2(NP_060818) c3(cap-specific mRNA (nucleoside-2′-0-)-methyltransferase 2) c4(197968) c5(197969, 197970, 197971, 197972, 197973, 197974, 197975, 197976, 197977, 197978) c6( ) c7(Y, kl, qK); #c1(CMYA5) c2(NP_705838) c3(cardiomyopathy-associated protein 5) c4(246319) c5(246320, 246321, 246322, 246323, 246324, 246325) c6( ) c7(oe, azk, km, cz, fq, af); #c1(CNBD1) c2(NP_775809) c3(cyclic nucleotide-binding domain-containing protein 1) c4(250382) c5(250383, 250384, 250385, 250386, 250387, 250388) c6( ) c7(bL); #c1(CNBP) c2(NP_001120664) c3(cellular nucleic acid-binding protein isoform 1) c4(44447) c5(44448, 44449, 44450, 44451, 44452, 44453) c6( ) c7(bL, T, dD, fM, qX, ea, axd, cz, bB, dG, j, aPl, v, eq, UX, GK, aT, jy, K, dQ, iU, xz, xs, fc, aPm, dq, aq, aaQ, aac, GR, dR, nT, cP, ei, lM, B, vQ); #c1 (CNBP) c2(NP_001120665) c3(cellular nucleic acid-binding protein isoform 2) c4(44454) c5(44455, 44456, 44457, 44458, 44459, 44460) c6( ) c7(bL, T, dD, fM, qX, ea, axd, cz, bB, dG, j, aPl, v, eq, UX, GK, aT, jy, K, dQ, iU, xz, xs, fc, aPm, dq, aq, aaQ, aac, GR, dR, nT, cP, ei, lM, B, vQ); #c1(CNBP) c2(NP_001120666) c3(cellular nucleic acid-binding protein isoform 4) c4(44461) c5(44462, 44463, 44464, 44465, 44466, 44467) c6( ) c7(bL, T, dD, fM, qX, ea, axd, cz, bB, dG, j, aPl, v, eq, UX, GK, aT, jy, K, dQ, iU, xz, xs, fc, aPm, dq, aq, aaQ, aac, GR, dR, nT, cP, ei, IM, B, vQ); #c1(CNBP) c2(NP_001120668) c3(cellular nucleic acid-binding protein isoform 6) c4(44468) c5(44469, 44470, 44471, 44472, 44473, 44474) c6( ) c7(bL, T, dD, fM, qX, ea, axd, cz, bB, dG, j, aPl, v, eq, UX, GK, aT, jy, K, de, iU, xz, xs, fc, aPm, dq, aq, aaO, aac, GR, dR, nT, cP, ei, lM, B, vQ); #c1(CNBP) c2(NP_003409) c3(cellular nucleic acid-binding protein isoform 3) c4(138238, 138239) c5(138240, 138241, 138242, 138243, 138244, 138245, 138246, 138247, 138248, 138249, 138250, 138251, 138252) c6( ) c7(bL, T, dD, fM, qX, ea, axd, cz, bB, dG, j, aPl, v, eq, UX, GK, aT, jy, K, dQ, iU, xz, xs, fc, aPm, dq, aq, aaO, aac, GR, dR, nT, cP, ei, lM, B, vQ); #c1(CNDP1) c2(NP_116038) c3(beta-Ala-His dipeptidase precursor) c4(225280) c5(225281, 225282, 225283, 225284, 225285, 225286) c6( ) c7(bL, T, A, eR, c, alp, al, fP, aj, dG, dy, j, nA, aY); #c1(CNDP2) c2(NP_001161971) c3(cytosolic non-specific dipeptidase isoform 2) c4(66309) c5(66310, 66311, 66312, 66313, 66314, 66315) c6( ) c7(bL, ck, T, A, aE, cA, eR, iL, rA, aA, aj, aF, dy, bt, Df, B, J); #c1(CNDP2) c2(XP_011524374) c3(cytosolic non-specific dipeptidase isoform X1) c4(319011) c5(319012, 319013, 319014, 319015, 319016, 319017) c6( ) c7(bL, ck, T, A, aE, cA, eR, iL, rA, aA, aj, aF, dy, bt, Df, B, J); #c1 (CNGA1) c2(NP_001136036) c3(cGMP-gated cation channel alpha- isoform 1) c4(54097, 54098, 54099) c5(54100, 54101, 54102, 54103, 54104, 54105, 54106, 54107, 54108, 54109, 54110, 54111, 54112) c6( ) c7(aQq, nC, lR); #c1(CNGA1) c2(XP_005248106) c3(cGMP-gated cation channel alpha-1 isoform X1) c4(268184) c5(268185, 268186, 268187, 268188, 268189, 268190, 268191, 268192, 268193, 268194, 268195, 268196, 268197, 268198, 268199, 268200, 268201, 268202, 268203, 268204, 268205, 268206, 268207, 268208, 268209, 268210, 268211) c6( ) c7(alq, nC, lR); #c1(CNGA3) c2(NP_001073347) c3(cyclic nucleotide-gated cation channel alpha-3 isoform 2) c4(35973) c5(35974, 35975, 35976, 35977, 35978, 35979) c6( ) c7(als, KE, lR, mA, nx, alr, nw, aQt, aQu, aCi); #c1(CNGB1) c2(NP_001129111) c3(cyclic nucleotide-gated cation channel beta-1 isoform b) c4(50799) c5(50800, 50801, 50802, 50803, 50804, 50805) c6( ) c7(sb, bM, alv, alx, lR, aDR, alw, nk, nC, oc, aDQ); #c1(CNGB1) c2(XP_006721197) c3(cyclic nucleotide-gated cation channel beta-1 isoform X1) c4(293889) c5(293890, 293891, 293892, 293893, 293894, 293895) c6( ) c7(sb, bM, alv, alx, lR, aDR, alw, nk, nC, oc, aDQ); #c1(CNGB3) c2(NP_061971) c3(cyclic nucleotide-gated cation channel beta-3) c4(201356) c5(201357, 201358, 201359, 201360, 201361, 201362) c6( ) c7(IR, nb, W, mA, alr, alx, nw, aQu, cd); #c1(CNIH3) c2(NP_689708) c3(protein cornichon homolog 3) c4(243653) c5(243654, 243655, 243656, 243657, 243658, 243659) c6( ) c7(dP, bM); #c1(CNKSR1) c2(NP_001284576) c3(connector enhancer of kinase suppressor of ras 1 isoform 2) c4(112070, 112071) c5(112072, 112073, 112074, 112075, (12076, 112077) c6( ) c7(Sw, Bl, T, aW, if, ha, aT, m, nA, ci, t, gT, qN, aly); #c1(CNKSR2) c2(NP_001162118) c3(connector enhancer of kinase suppressor of ras 2 isoform 2) c4(66320) c5(66321, 66322, 66323, 66324, 66325, 66326) c6( ) c7(W, v, aR); #c1(CNKSR2) c2(NP_001162119) c3(connector enhancer of kinase suppressor of ras 2 isoform 3) c4( ) c5(66327, 66328, 66329, 66330, 66331, 66332) c6( ) c7(W, v, aR); #c1(CNKSR2) c2(NP_001162120) c3(connector enhancer of kinase suppressor of ras 2 isoform 4) c4( ) c5(66333, 66334, 66335, 66336, 66337) c6( ) c7(W, v, aR); #c1(CNKSR2) c2(NP_055742) c3(connector enhancer of kinase suppressor of ras 2 isoform 1) c4(183221, 183222) c5(183223, 183224, 183225, 183226, 183227, 183228) c6( ) c7(W, v, aR); #c1(CNKSR2) c2(XP_011543774) c3(connector enhancer of kinase suppressor of ras 2 isoform X2) c4(342888) c5(342889) c6( ) c7(W, v, aR); #c1(CNKSR3) c2(NP_775786) c3(connector enhancer of kinase suppressor of ras 3) c4(250302, 250303) c5(250304, 250305, 250306, 250307, 250308, 250309, 250310) c6( ) c7(sk, bM, iM, je, aDy, Y, oe, Ne, bK, an, B, iP); #c1(CNKSR3) c2(XP_011533787) c3(connector enhancer of kinase suppressor of ras 3 isoform X1) c4(329927) c5(329928, 329929, 329930, 329931, 329932, 329933, 329934, 329935, 329936, 329937, 329938) c6( ) c7(sk, bM, iM, je, aDy, Y, oe, Ne, bK, an, B, iP); #c1(CNN1) c2(NP_001290) c3(calponin-1) c4( ) c5( ) c6( ) c7(B, X, mx, eH, K, uE, eF, cd); #c1(CNN1) c2(XP_011525991) c3(calponin-1 isoform X1) c4( ) c5(320363) c6( ) c7(B, X, mx, eH, K, uE, eF, cd); #c1(CNN1) c2(XP_011525992) c3(calponin-1 isoform X2) c4(320364, 320365) c5(320366) c6( ) c7(B, X, mx, eH, K, uE, eF, cd); #c1(CNN2) c2(NP_004359) c3(calponin-2 isoform a) c4(145655, 145656) c5(145657, 145658, 145659, 145660, 145661, 145662, 145663, 145664, 145665, 145666, 145667, 145668) c6( ) c7(By, ae, fr, ad); #c1(CNN2) c2(NP_958434) c3(calponin-2 isoform b) c4(262070) c5(262071, 262072, 262073, 262074, 262075, 262076) c6( ) c7(By, ae, fr, ad); #c1(CNNM1) c2(NP_065081) c3(metal transporter CNNM1) c4(203743, 203744, 203745) c5(203746, 203747, 203748, 203749, 203750, 203751, 203752, 203753, 203754, 203755, 203756, 203757, 203758, 203759, 203760, 203761, 203762, 203763) c6( ) c7(Ae, Y, ac, oj); #c1(CNNM1) c2(XP_005269749) c3(metal transporter CNNM1 isoform X3) c4(282746) c5(282747, 282748, 282749, 282750, 282751, 282752) c6( ) c7(Ae, Y, ac, oj): #c1(CNNM1) c2(XP_011537931) c3(metal transporter CNNM1 isoform X1) c4(335437) c5(335438, 335439, 335440, 335441, 335442, 335443) c6( ) c7(Ae, Y, ac, oj); #c1(CNNM2) c2(NP_060119) c3(metal transporter CNNM2 isoform 1) c4(194248, 194249) c5(194250, 194251, 194252, 194253, 194254, 194255, 194256, 194257, 194258, 194259, 194260, 194261) c6( ) c7(alz, Kk, cz, dr, nA, an, WI, ae, agZ, ad); #c1(CNNM2) c2(NP_951058) c3(metal transporter CNNM2 isoform 2) c4(261016, 261017) c5(261018, 261019, 261020, 261021, 261022, 261023, 261024, 261025, 261026, 261027, 261028, 261029) c6( ) c7(alz, Kk, cz, dr, nA, an, WI, ae, agZ, ad); #c1(CNNM2) c2(NP_951059) c3(metal transporter CNNM2 isoform 3) c4(261030, 261031) c5(261032, 261033, 261034, 261035, 261036, 261037, 261038, 261039, 261040, 261041, 261042, 261043) c6( ) c7(alz, Kk, cz, dr, nA, an, WI, ae, agZ, ad); #c1(CNNM4) c2(NP_064569) c3(metal transporter CNNM4 precursor) c4(203027) c5(203028, 203029, 203030, 203031, 203032, 203033) c6( ) c7(aQA, Rz, aQB, Ry, ol, nC, Ud); #c1(CNNM4) c2(XP_005263972) c3(metal transporter CNNM4 isoform X2) c4(278630) c5(278631, 278632, 278633, 278634, 278635, 278636) c6( ) c7(aQA, Rz, aQB, Ry, od, nC, Ud); #c1(CNOT1) c2(NP_001252541) c3(CCR4-NOT transcription complex subunit 1 isoform c) c4(94226) c5(94227, 94228, 94229, 94230, 94231, 94232, 94233, 94234, 94235, 94236, 94237) c6( ) c7(T, alC, d, EQ, fd, h, eA, hX); #c1(CNOT1) c2(NP_057368) c3(CCR4-NOT transcription complex subunit 1 isoform a) c4(190397, 190398, 190399) c5(190400, 190401, 190402, 190403, 190404, 190405, 190406, 190407, 190408, 190409, 190410, 190411, 190412, 190413, 190414, 190415, 190416, 190417, 190418) c6( ) c7(T, alC, d, EQ, fd, h, eA, hX); #c1(CNOT1) c2(NP_996882) c3(CCR4-NOT transcription complex subunit 1 isoform b) c4(264797, 264798) c5(264799, 264800, 264801, 264802, 264803, 264804) c6( ) c7(T, alC, d, EQ, fd, h, eA, hX); #c1(CNOT2) c2(NP_055330) c3(CCR4-NOT transcription complex subunit 2) c4(180521, 180522, 180523) c5(180524, 180525, 180526, 180527, 180528, 180529, 180530, 180531, 180532, 180533, 180534, 180535, 180536, 180537, 180538, 180539, 180540, 180541, 180542, 180543) c6( ) c7(bB, cd, bE, j, abW); #c1(CNOT2) c2(XP_006719492) c3(CCR4-NOT transcription complex subunit 2 isoform X2) c4(292568) c5( ) c6( ) c7(bB, cd, bE, j, abW); #c1(CNOT2) c2(XP_006719496) c3(CCR4-NOT transcription complex subunit 2 isoform X5) c4( ) c5(292569, 292570, 292571, 292572, 292573, 292574) c6( ) c7(bB, cd, bE, j, abW); #c1(CNOT2) c2(XP_011536703) c3(CCR4-NOT transcription complex subunit 2 isoform X6) c4(334247) c5(334248, 334249, 334250, 334251, 334252, 334253, 334254, 334255, 334256, 334257, 334258, 334259) c6( ) c7(bB, cd, bE, j, abW); #c1(CNOT3) c2(NP_055331) c3(CCR4-NOT transcription complex subunit 3) c4(180544, 180545) c5(180546, 180547, 180548, 180549, 180550, 180551, 180552, 180553, 180554, 180555, 180556, 180557, 180558, 180559, 180560, 180561, 180562, 180563, 180564, 180565, 180566, 180567, 180568, 180569, 180570, 180571, 180572, 180573, 180574, 180575, 180576, 180577, 180578) c6( ) c7(mR, s, eM, bj, nw, K, m, nC, cd); #c1(CNOT3) c2(XP_011545832) c3(CCR4-NOT transcription complex subunit 3 isoform X3) c4(344762) c5(344763, 344764, 344765, 344766, 344767, 344768) c6( ) c7(mR, s, eM, bj, nw, K, m, nC, cd); #c1(CNOT3) c2(XP_011545833) c3(CCR4-NOT transcription complex subunit 3 isoform X5) c4( ) c5(344769, 344770, 344771, 344772, 344773, 344774) c6( ) c7(mR, s, eM, bj, nw, K, m, nC, cd); #c1(CNOT4) c2(NP_001008226) c3(CCR4-NOT transcription complex subunit 4 isoform b) c4(22930) c5(22931, 22932, 22933, 22934, 22935, 22936) c6( ) c7(bz); #c1(CNOT4) c2(NP_001177776) c3(CCR4-NOT transcription complex subunit 4 isoform c) c4(73006) c5(73007, 73008, 73009, 73010, 73011, 73012) c6( ) c7(bz); #c1(CNOT4) c2(NP_001177777) c3(CCR4-NOT transcription complex subunit 4 isoform d) c4(73013, 73014) c5(73015, 73016, 73017, 73018, 73019, 73020, 73021, 73022, 73023, 73024, 73025, 73026, 73027, 73028, 73029, 73030) c6( ) c7(bz); #c1(CNOT4) c2(NP_001177778) c3(CCR4-NOT transcription complex subunit 4 isoform e) c4(73031) c5(73032, 73033, 73034, 73035, 73036, 73037) c6( ) c7(bz); #c1(CNOT4) c2(NP_001177779) c3(CCR4-NOT transcription complex subunit 4 isoform f) c4(73038, 73039) c5(73040, 73041, 73042, 73043, 73044, 73045, 73046, 73047, 73048, 73049, 73050, 73051) c6( ) c7(bz); #c1(CNOT4) c2(NP_037448) c3(CCR4-NOT transcription complex subunit 4 isoform a) c4(175565) c5(175566, 175567, 175568, 175569, 175570, 175571, 175572) c6( ) c7(bz); #c1 (CNOT6) c2(NP_001290170) c3(CCR4-NOT transcription complex subunit 6) c4(115010) c5(115011, 115012, 115013, 115014, 115015, 115016, 115017, 115018, 115019, 115020) c6( ) c7(bM, eX, CH, CL, V, aR, o, kH, h, eV, vB, bA, Nr, A, je, xR, Y, cr, eZ, aEz, po, eE, aEx, DF, Nq, cT, eU, iM, Jr, vZ, hc, mx, i, y, aEy); #c1(CNOT6L) c2(NP_653172) c3(CCR4-NOT transcription complex subunit 6-like) c4( ) c5(238935, 238936, 238937, 238938, 238939, 238940, 238941, 238942, 238943) c6( ) c7(bM); #c1(CNOT6L) c2(XP_011530109) c3(CCR4-NOT transcription complex subunit 6-like isoform X2) c4(326025) c5(326026) c6( ) c7(bM); #c1(CNOT7) c2(NP_037486) c3(CCR4-NOT transcription complex subunit 7 isoform 1) c4(175808, 175809, 175810) c5(175811, 175812, 175813, 175814, 175815, 175816) c6( ) c7(f, q, r, aT); #c1(CNOT7) c2(NP_473367) c3(CCR4-NOT transcription complex subunit 7 isoform 2) c4(230432) c5(230433, 230434, 230435, 230436, 230437, 230438, 230439, 230440, 230441, 230442, 230443, 230444, 230445, 230446, 230447, 230448, 230449, 230450) c6( ) c7(f, q, r, aT); #c1(CNOT8) c2(NP_001288003) c3(CCR4-NOT transcription complex subunit 8 isoform 2) c4(113366, 113367, 113368) c5(11369, 113370, 113371, 113372, 113373, 113374, 113375, 113376, 113377, 113378, 113379, 113380, 113381, 113382, 113383, 113384, 113385, 113386, 113387, 113388, 113389, 113390, 113391, 113392, 113393, 113394, 113395, 113396, 113397) c6( ) c7(lo, aT); #c1(CNOT8) c2(NP_001288011) c3(CCR4-NOT transcription complex subunit 8 isoform 3) c4(113398) c5(113399, 113400, 113401, 113402, 113403, 113404, 113405, 113406, 113407, 113408, 113409, 113410, 113411, 113412, 113413, 113414, 113415, 113416, 113417) c6( ) c7(lo, aT); #c1 (CNOT8) c2(NP_001288015) c3(CCR4-NOT transcription complex subunit 8 isoform 4) c4( ) c5(113425, 113426, 113427, 113428, 113429) c6( ) c7(lo, aT); #c1(CNOT8) c2(NP_004770) c3(CCR4-NOT transcription complex subunit 8 isoform 1) c4(149452, 149453, 149454, 149455, 149456, 149457, 149458, 149459) c5(149460, 149461, 149462, 149463, 149464, 149465, 149466, 149467, 149468, 149469, 149470, 149471, 149472, 149473, 149474, 149475, 149476, 149477, 149478, 149479, 149480, 149481, 149482, 149483, 149484, 149485, 149486, 149487, 149488, 149489, 149490, 149491, 149492, 149493, 149494, 149495, 149496, 149497, 149498, 149499, 149500, 149501, 149502, 149503) c6( ) c7(lo, aT); #c1(CNP) c2(NP_149124) c3(2′, 3′-cyclic-nucleotide 3′-phosphodiesterase) c4(227319, 227320, 227321) c5(227322, 227323, 227324, 227325, 227326, 227327, 227328, 227329) c6( ) c7(bM, dD, aQC, ea, cz, aNN, vr, GC, bS, ah, ct, eq, mF, V, Fc, cd, fm, rD, hP, he, af, d, aq, si, as, Z, Hv, B, vQ); #c1(CNP) c2(XP_011522642) c3(2′, 3′-cyclic-nucleotide 3′-phosphodiesterase isoform X1) c4(317180) c5(317181, 317182, 317183, 317184, 317185, 317186, 317187, 317188, 317189, 317190, 317191, 317192, 317193, 317194, 317195, 317196) c6( ) c7(bM, dD, aQC, ea, cz, aNN, vr, GC, bS, ah, ct, eq, mF, V, Fc, cd, fm, rD, hP, he, af, d, aq, si, as, Z, Hv, B, vQ); #c1(CNPY2) c2(NP_055070) c3(protein canopy homolog 2 isoform 1 precursor) c4(178747, 178748) c5(178749, 178750, 178751, 178752, 178753, 178754, 178755, 178756, 178757, 178758, 178759, 178760) c6( ) c7(aA); #c1(CNPY3) c2(NP_006577) c3(protein canopy homolog 3 precursor) c4(166343, 166344) c5(166345, 166346, 166347, 166348, 166349, 166350, 166351, 166352, 166353, 166354, 166355) c6( ) c7(U, kB); #c1(CNPY3) c2(XP_005248874) c3(protein canopy homolog 3 isoform X4) c4( ) c5(268710) c6( ) c7(U, kB); #c1(CNR1) c2(NP_001153731) c3(cannabinoid receptor 1 isoform a) c4(60917, 60918) c5(60919, 60920, 60921, 60922, 60923, 60924, 60925, 60926, 60927, 60928, 60929, 60930, 60931, 60932, 60933, 60934, 60935, 60936, 60937, 60938, 60939, 60940, 60941, 60942, 60943, 60944, 60945, 60946, 60947, 60948, 60949, 60950) c6( ) c7(T, uk, aC, ea, e, al, lr, bO, gf, VV, aQF, bv, Bc, iX, K, qM, dQ, aW, fc, hl, aB, as, cm, eM, Gs, U, vy, sv, Xu, dD, aw, aj, ct, Vs, lG, jy, eV, he, ge, fQ, fe, kp, hq, ar, aap, ok, hk, Ei, lB, cB, abW, B, jd, bL, Dh, hU, jx, eX, cz, v, j, il, V, q, J, eA, eJ, uT, DK, rS, aql, HC, aT, bk, bq, an, ae, bA, Y, yL, r, cu, HL, xw, FT, bN, pH, rR, vK, ac, mf, jh, Gd, bS, uu, jE, eq, qi, ad, tX, dP, HF, aDE, F, afm, Sb, Gv, gC, gl, dq, iV, Z, jb, HP, aQG, gZ, Rm, ei, ra, y, aqw, xg, iu, aQD); #c1(CNR1) c2(NP_149421) c3(cannabinoid receptor 1 isoform b) c4(227653, 227654) c5(227655, 227656, 227657, 227658, 227659, 227660, 227661, 227662, 227663, 227664, 227665, 227666) c6( ) c7(T, uk, aC, ea, e, al, lr, bO, gf, VV, aQF, bv, Bc, iX, K, qM, dQ, aW, fc, hl, aB, as, cm, eM, Gs, U, vy, sv, Xu, dD, aw, aj, ct, Vs, lG, jy, eV, he, ge, fQ, fe, kp, hq, ar, aap, ok, hk, Ei, lB, cB, abW, B, jd, bL, Dh, hU, jx, eX, cz, v, j, il, V, q, J, eA, eJ, uT, DK, rS, aql, HC, aT, bK, bq, an, ae, bA, Y, yL, r, cu, HL, xw, FT, bN, pH, rR, vK, ac, mf, jh, Gd, bS, uu, jE, eq, qi, ad, tX, dP, HF, aDE, F, afm, Sb, Gv, gC, gl, dq, iV, Z, jb, HP, aQG, gZ, Rm, ei, ra, y, aqw, xg, iu, aQD); #c1(CNR2) c2(XP_011538931) c3(cannabinoid receptor 2 isoform X1) c4(337003) c5(337004, 337005, 337006, 337007, 337008, 337009, 337010, 337011, 337012, 337013) c6( ) c7(bL, aC, dD, ea, e, My, al, jx, aw, bS, eq, ct, V, cd, ad, ai, eJ, ke, F, Bc, aT, cy, bK, K, ji, DY, af, bc, bA, aW, hl, zD, fe, gu, Z, jT, cm, jb, pb, ck, ar, kT, ok, rR, c, nB, yE, hk, Gs, U, bt, eo, y, lB, aen, B, vi); #c1(CNRIP1) c2(NP_001104571) c3(CBI cannabinoid receptor-interacting protein 1 isoform CRIP1b) c4(42186) c5(42187, 42188, 42189, 42190, 42191, 42192) c6( ) c7(bM, T, s, aW, eH, aT, j, eF); #c1(CNRIP1) c2(NP_056278) c3(CBI cannabinoid receptor-interacting protein 1 isoform CRIP1a) c4(186900) c5(186901, 186902, 186903, 186904, 186905, 186906) c6( ) c7(bM, T, s, aW, eH, aT, j, eF); #c1(CNST) c2(NP_001132931) c3(consortin isoform 2) c4(52674, 52675) c5(52676, 52677, 52678, 52679, 52680, 52681, 52682, 52683, 52684, 52685, 52686, 52687) c6( ) c7(hz); #c1(CNST) c2(NP_689822) c3(consortin isoform 1) c4(244023) c5(244024, 244025, 244026, 244027, 244028, 244029) c6( ) c7(hz); #c1(CNTF) c2(NP_000605) c3(ciliary neurotrophic factor) c4(13678) c5(13679, 13680, 13681, 13682, 13683, 13684, 13685) c6(13686) c7(cZ, lT, Ol, eX, ti, aj, bS, Vd, ct, gf, PJ, fu, dG, V, bX, v, J, eA, eW, ke, fm, aql, HC, eV, aaw, bu, dd, yo, d, lR, s, aq, as, dw, Z, nC, cm, bJ, ck, rR, W, c, U, nw, eo, B, de); #c1(CNTFR) c2(NP_001193940) c3(ciliary neurotrophic factor receptor subunit alpha preproprotein) c4(83258, 83259) c5(83260, 83261, 83262, 83263, 83264, 83265, 83266, 83267, 83268, 83269) c6( ) c7(hM, af, c, aql, aQH, eA, bJ); #c1(CNTFR) c2(XP_011516009) c3(ciliary neurotrophic factor receptor subunit alpha isoform X1) c4( ) c5(309221, 309222, 309223, 309224, 309225, 309226) c6( ) c7(hM, af, c, aql, aQH, eA, bJ); #c1(CNTLN) c2(NP_001107867) c3(centlein isoform 2) c4(43054) c5(43055, 43056, 43057, 43058, 43059, 43060) c6( ) c7(bM, T, nD, ago, ac, QK, et, dB, aW, aT, gZ, dz, aj, m, Nh, t, ik, B, J); #c1(CNTLN) c2(NP_060208) c3(centlein isoform 1) c4(194747, 194748) c5(194749, 194750, 194751, 194752, 194753, 194754, 194755, 194756, 194757, 194758, 194759, 194760) c6( ) c7(bM, T, nD, ago, ac, QK, et, dB, aW, aT, gZ, dz, aj, m, Nh, t, iK, B, J); #c1(CNTLN) c2(XP_011516242) c3(centlein isoform X2) c4(309475) c5(309476, 309477, 309478, 309479, 309480, 309481) c6( ) c7(bM, T, nD, ago, ac, QK, et, dB, aW, aT, gZ, dz, aj, m, Nh, t, ik, B, J); #c1(CNTN1) c2(NP_001242993) c3(contactin-1 isoform 3 precursor) c4(89466, 89467) c5(89468, 89469, 89470, 89471, 89472, 89473, 89474, 89475, 89476, 89477, 89478, 89479, 89480, 89481, 89482, 89483, 89484, 89485, 89486, 89487, 89488, 89489, 89490, 89491, 89492, 89493, 89494, 89495, 89496, 89497, 89498, 89499, 89500, 89501, 89502, 89503, 89504, 89505) c6( ) c7(iu, aw, ht, ff, oj, Ad, en, hu, g, aU, bz, eA); #c1(CNTN1) c2(NP_778203) c3(contactin-1 isoform 2 precursor) c4(251375) c5(251376, 251377, 251378, 251379, 251380, 251381) c6( ) c7(iu, aw, ht, ff, oj, Ad, en, hu, g, aU, bz, eA); #c1(CNTN1) c2(XP_011536229) c3(contactin-1 isoform X1) c4(333544, 333545, 333546, 333547) c5(333548, 333549, 333550, 333551, 333552, 333553, 333554, 333555, 333556, 333557) c6( ) c7(iu, aw, ht, ff, oj, Ad, en, hu, g, aU, bz, eA); #c1(CNTN2) c2(NP_005067) c3(contactin-2 precursor) c4(152573) c5(152574, 152575, 152576, 152577, 152578, 152579) c6( ) c7(T, fM, dA, ha, fN, Fc, pd, ai, Pm, aQJ, eA, rw, kH, Ne, h, rB, mH, d, je, Y, An, as, QK, t, alx, ans, ck, iM, ac, Ew, apO, yW, aiS, m, y); #c1(CNTN3) c2(NP_065923) c3(contactin-3 precursor) c4(206279) c5(206280, 206281, 206282, 206283, 206284, 206285) c6( ) c7(ck, cc, anR, aW, fJ, aT, fd, j, ae, ad, bN, mu); #c1(CNTN4) c2(NP_001193884) c3(contactin-4 isoform a precursor) c4(83022, 83023, 83024) c5(83025, 83026, 83027, 83028, 83029, 83030, 83031, 83032, 83033, 83034, 83035, 83036, 83037, 83038, 83039, 83040, 83041, 83042, 83043, 83044, 83045, 83046, 83047, 83048, 83049, 83050, 83051) c6( ) c7(c, kB, dH, oe, Az, fq, Vd, bK, nA, hB, rz, xb, bN); #c1 (CNTN4) c2(NP_783302) c3(contactin-4 isoform c) c4(251593) c5(251594, 251595, 251596, 251597, 251598, 251599, 251600, 251601) c6( ) c7(c, kB, dH, oe, Az, fq, Vd, bK, nA, hB, rz, xb, bN); #c1(CNTN4) c2(XP_006713067) c3(contactin-4 isoform X2) c4( ) c5(287009, 287010, 287011, 287012, 287013, 287014, 287015) c6( ) c7(c, kB, dH, oe, Az, fq, Vd, bK, nA, hB, rz, xb, bN); #c1(CNTN4) c2(XP_011531733) c3(contactin-4 isoform X3) c4(327824) c5( ) c6( ) c7(c, kB, dH, oe, Az, fq, Vd, bK, nA, hB, rz, xb, bN); #c1(CNTN5) c2(NP_001230199) c3(contactin-5 isoform 1 precursor) c4(86095) c5(86096, 86097, 86098, 86099, 86100, 86101, 86102, 86103, 86104, 86105, 86106, 86107, 86108, 86109) c6( ) c7(eW, ac, af, rz, hb, wL, aw, Ak, vr, aur, nA, he, KI, cd, cY); #c1(CNTN5) c2(NP_001230200) c3(contactin-5 isoform 3 precursor) c4(86110) c5(86111, 86112, 86113, 86114, 86115, 86116) c6( ) c7(eW, ac, af, rz, hb, wL, aw, Ak, vr, aur, nA, he, KI, cd, cY); #c1(CNTN5) c2(XP_011541173) c3(contactin-5 isoform X1) c4(339779) c5(339780, 339781, 339782, 339783, 339784, 339785) c6( ) c7(eW, ac, af, rz, hb, wL, aw, Ak, vr, aur, nA, he, Kl, cd, cY); #c1(CNTN6) c2(NP_001276010) c3(contactin-6 isoform 2) c4(107735) c5( ) c6( ) c7(aw, qK, Ak, bK, nA, he, Kl, ad, cY); #c1(CNTN6) c2(NP_055276) c3(contactin-6 isoform 1 precursor) c4(180212) c5(180213, 180214, 180215, 180216, 180217, 180218, 180219, 180220, 180221, 180222, 180223, 180224, 180225, 180226, 180227, 180228, 180229) c6( ) c7(aw, qK, Ak, bK, nA, he, Kl, ad, cY); #c1(CNTNAP1) c2(NP_003623) c3(contactin-associated protein 1 precursor) c4(139695) c5(139696, 139697, 139698, 139699, 139700, 139701) c6( ) c7(T, ow, DE, oD, jh, ol, hF, Fc, eA, br, x, aE, oC, OG, dV, aT, oF, hn, ox, iL, iU, bw, bN, ck, pc, pg, k, eg, bt); #c1(CNTNAP2) c2(NP_054860) c3(contactin-associated protein-like 2 precursor) c4(178117, 178118) c5(178119, 178120, 178121, 178122, 178123, 178124, 178125) c6( ) c7(aQP, T, wY, ahc, FG, Or, ha, al, VT, cz, Ir, bO, aj, aQN, nA, aQK, cd, aR, eA, hX, ft, dY, hb, aQQ, Ko, aQM, bK, he, rz, af, na, bA, VX, yL, aOO, aha, JI, aQL, fq, bN, pH, gk, ac, vD, Kr, k, yA, hh, qc); #c1(CNTNAP2) c2(XP_006715982) c3(contactin-associated protein-like 2 isoform X1) c4( ) c5(290002, 290003, 290004, 290005, 290006) c6( ) c7(aQP, T, wY, ahc, FG, Or, ha, al, VT, cz, lr, bO, aj, aQN, nA, aQK, cd, aR, eA, hX, ft, dY, hb, aQQ, Ko, aQM, bK, he, rz, af, na, bA, VX, yL, aQO, aha, JI, aQL, fq, bN, pH, gk, ac, vD, Kr, k, yA, hh, qc); #c1(CNTNAP3) c2(NP_387504) c3(contactin-associated protein-like 3 precursor) c4(228984, 228985) c5(228986, 228987, 228988, 228989, 228990, 228991, 228992, 228993, 228994, 228995, 228996, 228997, 228998, 228999, 229000, 229001) c6(229002, 229003, 229004, 229005) c7(T); #c1(CNTNAP3) c2(XP_006716934) c3(contactin-associated protein-like 3 isoform X1) c4(290565, 290566) c5(290567, 290568, 290569, 290570, 290571, 290572, 290573, 290574, 290575, 290576, 290577, 290578, 290579, 290580, 290581, 290582) c6(290583, 290584, 290585, 290586) c7(T); #c1(CNTNAP3) c2(XP_006716936) c3(contactin-associated protein-like 3 isoform X4) c4(290587) c5(290588, 290589, 290590, 290591, 290592, 290593, 290594, 290595) c6(290596, 290597) c7(T); #c1(CNTNAP3) c2(XP_011516335) c3(contactin-associated protein-like 3 isoform X1) c4( ) c5(309544, 309545, 309546, 309547, 309548, 309549, 309550, 309551, 309552, 309553, 309554, 309555) c6( ) c7(T); #c1(CNTNAP4) c2(NP_207837) c3(contactin-associated protein-like 4 isoform 1 precursor) c4(228512, 228513) c5(228514, 228515, 228516, 228517, 228518, 228519, 228520, 228521, 228522, 228523, 228524, 228525) c6( ) c7(he, aj, j, dH, aT); #c1(CNTNAP4) c2(NP_620481) c3(contactin-associated protein-like 4 isoform 2) c4(237694) c5(237695, 237696, 237697, 237698, 237699, 237700) c6( ) c7(he, aj, j, dH, aT); #c1(CNTNAP5) c2(NP_570129) c3(contactin-associated protein-like 5 precursor) c4(234025) c5(234026, 234027, 234028, 234029, 234030, 234031) c6( ) c7(ac, hb, Y, oe, n, bK, yA, fq, bN); #c1(CNTRL) c2(XP_005251736) c3(centriolin isoform X1) c4(271104, 271105, 271106, 271107, 271108) c5(271109, 271110, 271111, 271112, 271113, 271114, 271115, 271116, 271117, 271118, 271119, 271120, 271121, 271122, 271123, 271124, 271125, 271126, 271127, 271128, 271129, 271130, 271131, 271132, 271133, 271134, 271135, 271136, 271137) c6( ) c7(T, et, Y, aT, dz, y, eA); #c1(CNTRL) c2(XP_006717003) c3(centriolin isoform X6) c4( ) c5(290631, 290632, 290633, 290634, 290635, 290636) c6( ) c7(T, et, Y, aT, dz, y, eA); #c1(CNTRL) c2(XP_006717005) c3(centriolin isoform X8) c4(290637) c5( ) c6( ) c7(T, et, Y, aT, dz, y, eA); #c1(CNTRL) c2(XP_006717007) c3(centriolin isoform X10) c4(290638) c5(290639, 290640, 290641, 290642, 290643, 290644, 290645, 290646, 290647, 290648, 290649) c6( ) c7(T, et, Y, aT, dz, y, eA); #c1(CNTRL) c2(XP_011516469) c3(centriolin isoform X2) c4( ) c5(309802) c6( ) c7(T, et, Y, aT, dz, y, eA); #c1(CNTRL) c2(XP_011516471) c3(centriolin isoform X4) c4(309803) c5( ) c6( ) c7(T, et, Y, aT, dz, y, eA); #c1(CNTRL) c2(XP_011516473) c3(centriolin isoform X7) c4(309804) c5( ) c6( ) c7(T, et, Y, aT, dz, y, eA); #c1(CNTRL) c2(XP_011516474) c3(centriolin isoform X9) c4(309805, 309806) c5(309807, 309808, 309809, 309810, 309811, 309812, 309813, 309814, 309815, 309816, 309817, 309818) c6( ) c7(T, et, Y, aT, dz, y, eA); #c1(CNTRL) c2(XP_011516475) c3(centriolin isoform X1) c4( ) c5(309819, 309820, 309821, 309822, 309823, 309824, 309825, 309826, 309827, 309828, 309829) c6( ) c7(T, et, Y, aT, dz, y, eA); #c1(CNTROB) c2(NP_001032221) c3(centrobin isoform beta) c4(31104) c5(31105, 31106, 31107, 31108, 31109, 31110) c6( ) c7(aW, kl, aiB, aT); #c1(CNTROB) c2(NP_444279) c3(centrobin isoform alpha) c4(230252, 230253) c5(230254, 230255, 230256, 230257, 230258, 230259) c6( ) c7(aW, kl, aiB, aT); #c1(CNTROB) c2(XP_006721502) c3(centrobin isoform X1) c4( ) c5(294010, 294011) c6( ) c7(aW, kl, aiB, aT); #c1(CNTROB) c2(XP_011521936) c3(centrobin isoform X6) c4( ) c5(316638, 316639, 316640, 316641, 316642, 316643) c6( ) c7(aW, kl, aiB, aT); #c1(COA1) c2(NP_060694) c3(cytochrome c oxidase assembly factor 1 homolog) c4(197222, 197223, 197224) c5(197225, 197226, 197227, 197228, 197229, 197230, 197231, 197232, 197233, 197234, 197235, 197236, 197237, 197238, 197239, 197240, 197241, 197242, 197243, 197244, 197245, 197246, 197247, 197248, 197249, 197250, 197251, 197252, 197253, 197254, 197255, 197256, 197257, 197258, 197259, 197260, 197261, 197262, 197263, 197264, 197265, 197266, 197267, 197268, 197269, 197270) c6( ) c7(avQ, BE); #c1(COA5) c2(NP_001008216) c3(cytochrome c oxidase assembly factor 5) c4(22890) c5(22891, 22892, 22893, 22894, 22895, 22896) c6( ) c7(vc, cc, acJ, p); #c1(COASY) c2(NP_001035997) c3(bifunctional coenzyme A synthase isoform c) c4(33635, 33636) c5(33637, 33638, 33639, 33640, 33641, 33642, 33643, 33644, 33645, 33646, 33647, 33648) c6( ) c7(if, adi, ml, qN, adf, axa, acX, zw, aQS, aT, dL, K, EL, aQR, avH, aq, cm, iP, EJ, acW, bt, xg, AN, ao); #c1(COASY) c2(NP_079509) c3(bifunctional coenzyme A synthase isoform a precursor) c4(218429) c5(218430, 218431, 218432, 218433, 218434, 218435, 218436, 218437, 218438, 218439, 218440, 218441, 218442, 218443, 218444, 218445) c6( ) c7(if, adi, ml, qN, adf, axa, acX, zw, aQS, aT, dL, K, EL, aQR, avH, aq, cm, iP, EJ, acW, bt, xg, AN, ao); #c1(COASY) c2(XP_011523603) c3(bifunctional coenzyme A synthase isoform X3) c4(318085) c5(318086, 318087, 318088, 318089) c6( ) c7(if, adi, ml, qN, adf, axa, acX, zw, aQS, aT, dL, K, EL, aQR, avH, aq, cm, iP, EJ, acW, bt, xg, AN, ao); #c1(COBL) c2(NP_001274367) c3(protein cordon-bleu isoform c) c4(106292) c5(106293, 106294, 106295, 106296, 106297, 106298, 106299, 106300, 106301, 106302, 106303, 106304) c6( ) c7(A, EQ, cz, cd, bX, cm, sD, af); #c1(COBL) c2(NP_056013) c3(protein cordon-bleu isoform b) c4(185133) c5(185134, 185135, 185136, 185137, 185138, 185139) c6( ) c7(A, EI, cz, cd, bX, cm, sD, af); #c1(COBL) c2(XP_005271813) c3(protein cordon-bleu isoform X10) c4(283604) c5( ) c6( ) c7(A, EQ, cz, cd, bX, cm, sD, af); #c1(COBL) c2(XP_011513537) c3(protein cordon-bleu isoform X2) c4(305811, 305812) c5(305813, 305814, 305815, 305816, 305817, 305818, 305819, 305820, 305821, 305822, 305823, 305824) c6( ) c7(A, EQ, cz, cd, bX, cm, sD, af); #c1(COBL) c2(XP_011513539) c3(protein cordon-bleu isoform X6) c4(305825) c5( ) c6( ) c7(A, EQ, cz, cd, bX, cm, sD, af); #c1(COBL) c2(XP_011513543) c3(protein cordon-bleu isoform X11) c4( ) c5(305826, 305827, 305828, 305829, 305830, 305831) c6( ) c7(A, EQ, cz, cd, bX, cm, sD, af); #c1(COBLL1) c2(NP_001265387) c3(cordon-bleu protein-like 1 isoform 1) c4(98413, 98414) c5(98415, 98416, 98417, 98418, 98419, 98420, 98421, 98422, 98423, 98424, 98425, 98426) c6( ) c7(v, j, ac); #c1(COBLL1) c2(NP_001265389) c3(cordon-bleu protein-like 1 isoform 3) c4(98427, 98428, 98429) c5(98430, 98431, 98432, 98433, 98434, 98435, 98436, 98437, 98438, 98439, 98440, 98441) c6( ) c7(v, j, ac); #c1(COBLL1) c2(NP_001265390) c3(cordon-bleu protein-like 1 isoform 4) c4(98442) c5(98443, 98444, 98445, 98446, 98447, 98448, 98449, 98450, 98451, 98452, 98453, 98454) c6( ) c7(v, j, ac); #c1(COBLL1) c2(NP_055715) c3(cordon-bleu protein-like 1 isoform 2) c4(183016) c5(183017, 183018, 183019, 183020, 183021, 183022) c6( ) c7(v, j, ac); #c1(COCH) c2(XP_011534841) c3(cochlin isoform X1) c4(331337, 331338, 331339) c5(331340, 331341, 331342, 331343, 331344, 331345, 331346, 331347, 331348, 331349, 331350, 331351, 331352, 331353, 331354, 331355, 331356, 331357, 331358, 331359, 331360, 331361) c6( ) c7(aQU, qk, b, alX, aQT, Fh, i, mG, aRa, U, aQV, K, aQW, dw, akv, Bh, aQY, alZ); #c1(COG1) c2(NP_061184) c3(conserved oligomeric Golgi complex subunit 1) c4(199837, 199838) c5(199839, 199840, 199841, 199842, 199843, 199844, 199845, 199846, 199847, 199848, 199849, 199850) c6( ) c7(aRd, SP, aRb, aRc, pk, af); #c1(COG2) c2(NP_001138508) c3(conserved oligomeric Golgi complex subunit 2 isoform 2) c4(56912) c5(56913, 56914, 56915, 56916, 56917, 56918) c6( ) c7(ZV, ac, cH, e, aw, j, af, eu); #c1(COG2) c2(NP_031383) c3(conserved oligomeric Golgi complex subunit 2 isoform 1) c4(172124, 172125, 172126, 172127) c5(172128, 172129, 172130, 172131, 172132, 172133, 172134, 172135, 172136, 172137, 172138, 172139, 172140, 172141, 172142, 172143, 172144, 172145, 172146, 172147, 172148, 172149, 172150, 172151) c6( ) c7(ZV, ac, cH, e, aw, j, af, eu); #c1(COG3) c2(NP_113619) c3(conserved oligomeric Golgi complex subunit 3) c4(221760) c5(221761, 221762, 22/763, 221764, 221765, 221766) c6( ) c7(atO); #c1(COG4) c2(NP_001182068) c3(conserved oligomeric Golgi complex subunit 4 isoform 2) c4(74539) c5(74540, 74541, 74542, 74543, 74544, 74545, 74546, 74547, 74548, 74549, 74550, 74551) c6( ) c7(aRe, nC, OL, SP); #c1(COG4) c2(NP_056201) c3(conserved oligomeric Golgi complex subunit 4 isoform 1) c4(186293, 186294) c5(186295, 186296, 186297, 186298, 186299, 186300, 186301) c6( ) c7(aRe, nC, OL, SP); #c1(COG4) c2(XP_011521283) c3(conserved oligomeric Golgi complex subunit 4 isoform X1) c4(316/77) c5(316178, 316179, 316180, 316181, 316182) c6( ) c7(aRe, nC, OL, SP); #c1(COG5) c2(NP_006339) c3(conserved oligomeric Golgi complex subunit 5 isoform 1) c4(164154, 164155) c5(164156, 164157, 164158, 164159, 164160, 164161, 164162, 164163, 164164, 164165, 164166, 164167) c6( ) c7(aRf, SL); #c1(COG5) c2(NP_859422) c3(conserved oligomeric Golgi complex subunit 5 isoform 2) c4(255529) c5(255530, 255531, 255532, 255533, 255534, 255535) c6( ) c7(aRf, SL); #c1(COG6) c2(NP_001138551) c3(conserved oligomeric Golgi complex subunit 6 isoform 2) c4(56990) c5(56991, 56992, 56993, 56994, 56995, 56996) c6( ) c7(ant, iV, Jr, aRg, oe, dv, aRh, cr, la, Lo, OL, Xk); #c1(COG6) c2(NP_065802) c3(conserved oligomeric Golgi complex subunit 6 isoform 1) c4(205596, 205597) c5(205598, 205599, 205600, 205601, 205602, 205603, 205604, 205605, 205606, 205607, 205608, 205609) c6( ) c7(ant, iV, Jr, aRg, oe, dv, aRh, cr, la, Lo, OL, Xk); #c1(COG7) c2(NP_705831) c3(conserved oligomeric Golgi complex subunit 7) c4(246289) c5(246290, 246291, 246292, 246293, 246294, 246295) c6( ) c7(aRi, Lo, Km, QK); #c1(COG8) c2(NP_115758) c3(conserved oligomeric Golgi complex subunit 8) c4(223838) c5(223839, 223840, 223841, 223842, 223843, 223844) c6( ) c7(SL, aRj, SP); #c1(COIL) c2(NP_004636) c3(coilin) c4(148403) c5(148404, 148405, 148406, 148407, 148408, 148409) c6( ) c7(ck, b, XE, i, eH, aRk, jh, kf, m, eF, t, nJ); #c1(COL10A1) c2(XP_011533735) c3(collagen alpha-f(X) chain isoform X1) c4(329818, 329819, 329820) c5(329821, 329822, 329823, 329824, 329825, 329826, 329827, 329828, 329829, 329830, 329831, 329832, 329833, 329834, 329835, 329836, 329837, 329838, 329839, 329840, 329841, 329842, 329843, 329844) c6( ) c7(kF, W, rl, ln, aRm, aT, aRl, rD, ade, rv, aRn, cY, aRo); #c1(COL11A1) c2(XP_011539021) c3(collagen alpha-1(X1) chain isoform X1) c4( ) c5(337085, 337086, 337087, 337088, 337089, 337090, 337091, 337092, 337093) c6(337094, 337095) c7(T, aRu, n, aRt, q, Na, aE, rl, By, Nc, sa, rv, aRs, s, ff, Az, r, en, mo, dw, aRq, t, g, iK, Ek, bF, aik, aRp, eM, BP, ln, ro, aRr, m, lJ); #c1(COL11A1) c2(XP_011539023) c3(collagen alpha-1(X1) chain isoform X3) c4(337096) c5(337097, 337098, 337099, 337100, 337101, 337102, 337103, 337104) c6(337105, 337106) c7(T, aRu, n, aRt, q, Na, aE, rl, By, Nc, sa, rv, aRs, s, ff, Az, r, en, mo, dw, aRq, t, g, ik, Ek, bF, aik, aRp, eM, BP, ln, ro, aRr, m, lJ); #c1 (COL11A2) c2(NP_001157243) c3(collagen alpha-2(X1) chain isoform 4 precursor) c4(62093, 62094, 62095) c5(62096, 62097, 62098, 62099, 62100, 62101, 62102, 62103, 62104, 62105, 62106, 62107, 62108, 62109, 62110, 62/11, 62112, 62113, 62114, 62115, 62116, 62117, 62118, 62119, 62120, 62121, 62122, 62123, 62124, 62125, 62126, 62127, 62128, 62129) c6(62130, 62131, 62132, 62133) c7(eW, cZ, os, DM, T, WL, aRx, NT, fd, bB, jh, aj, oZ, fr, ci, q, J, cd, ad, aRo, Na, aRy, hd, no, aE, rl, aT, h, Nc, K, dd, hS, rv, ae, af, vl, A, d, aW, eH, Az, eZ, r, aq, aRw, kp, eF, t, cm, bz, atb, kP, eM, nB, bE, BP, mG, aRz, ln, afd, cj, aef, asR, ro, aRA, m, y, bt, Bh, B, aRv); #c1(COL11A2) c2(NP_542410) c3(collagen alpha-2(X1) chain isoform 3 preproprotein) c4(232408, 232409, 232410, 232411, 232412, 232413, 232414, 232415) c5(232416, 232417, 232418, 232419, 232420, 232421, 232422, 232423, 232424, 232425, 232426, 232427, 232428, 232429, 232430, 232431, 232432, 232433, 232434, 232435, 232436, 232437, 232438, 232439, 232440, 232441, 232442, 232443, 232444, 232445, 232446, 232447, 232448, 232449, 232450, 232451, 232452, 232453, 232454) c6(232455, 232456, 232457, 232458) c7(eW, cZ, os, DM, T, WL, aRx, NT, fd, bB, jh, aj, oZ, fr, ci, q, J, cd, ad, aRo, Na, aRy, hd, no, aE, rl, aT, h, Nc, K, dd, hS, rv, ae, af, vl, A, d, aW, eH, Az, eZ, r, aq, aRw, kp, ef, t, cm, bz, atb, kP, eM, nB, bE, BP, mG, aRz, ln, afd, cj, aef, asR, ro, aRA, m, y, bt, Bh, B, aRv); #c1(COL11A2) c2(NP_542411) c3(collagen alpha-2(X1) chain isoform 1 preproprotein) c4(232459, 232460, 232461, 232462, 232463, 232464, 232465, 232466, 232467, 232468, 232469, 232470) c5(232471, 232472, 232473, 232474, 232475, 232476, 232477, 232478, 232479, 232480, 232481, 232482, 232483, 232484, 232485, 232486, 232487, 232488, 232489, 232490, 232491, 232492, 232493, 232494, 232495, 232496, 232497, 232498, 232499, 232500, 232501, 232502, 232503, 232504, 232505, 232506, 232507, 232508, 232509, 232510, 232511, 232512, 232513, 232514, 232515, 232516, 232517, 232518, 232519, 232520, 232521, 232522, 232523, 232524, 232525, 232526, 232527, 232528, 232529, 232530, 232531, 232532, 232533, 232534, 232535, 232536, 232537, 232538, 232539, 232540, 232541, 232542, 232543, 232544, 232545, 232546, 232547, 232548, 232549, 232550, 232551, 232552, 232553, 232554, 232555, 232556, 232557, 232558, 232559, 232560, 232561, 232562, 232563, 232564, 232565, 232566, 232567, 232568, 232569, 232570, 232571, 232572, 232573, 232574, 232575, 232576, 232577, 232578, 232579, 232580, 232581, 232582, 232583, 232584, 232585, 232586, 232587, 232588, 232589, 232590, 232591, 232592, 232593, 232594, 232595, 232596, 232597, 232598, 232599, 232600, 232601, 232602, 232603, 232604, 232605, 232606, 232607, 232608, 232609, 232610, 232611, 232612, 232613, 232614, 232615, 232616, 232617, 232618, 232619, 232620, 232621, 232622, 232623, 232624, 232625, 232626, 232627, 232628, 232629, 232630, 232631, 232632, 232633, 232634, 232635, 232636, 232637, 232638, 232639, 232640, 232641, 232642, 232643, 232644, 232645, 232646, 232647, 232648, 232649) c6(232650, 232651, 232652, 232653, 232654, 232655, 232656, 232657, 232658, 232659, 232660, 232661, 232662, 232663, 232664, 232665, 232666, 232667, 232668, 232669) c7(eW, cZ, os, DM, T, WL, aRx, NT, fd, bB, jh, aj, oZ, fr, ci, q, J, cd, ad, aRo, Na, aRy, hd, no, aE, rl, aT, h, Nc, K, dd, hS, rv, ae, af, vl, A, d, aW, eH, Az, eZ, r, aq, aRw, kp, eF, t, cm, bz, atb, kP, eM, nB, bE, BP, mG, aRz, ln, afd, cj, aef, asR, ro, aRA, m, y, bt, Bh, B, aRv); #c1(COL11A2) c2(NP_542412) c3 (collagen alpha-2(X1) chain isoform 2 preproprotein) c4(232670, 232671, 232672, 232673) c5(232674, 232675, 232676, 232677, 232678, 232679, 232680, 232681, 232682, 232683, 232684, 232685, 232686, 232687, 232688, 232689, 232690, 232691, 232692, 232693, 232694, 232695, 232696, 232697, 232698, 232699, 232700, 232701, 232702, 232703, 232704, 232705, 232706, 232707, 232708, 232709, 232710, 232711, 232712, 232713, 232714, 232715, 232716, 232717, 232718, 232719, 232720, 232721, 232722, 232723, 232724, 232725, 232726, 232727, 232728, 232729, 232730, 232731, 232732, 232733, 232734, 232735, 232736, 232737, 232738, 232739, 232740, 232741, 232742, 232743, 232744, 232745, 232746, 232747, 232748, 232749, 232750, 232751, 232752, 232753, 232754, 232755, 232756, 232757, 232758, 232759, 232760, 232761, 232762, 232763, 232764, 232765, 232766, 232767, 232768, 232769, 232770, 232771, 232772, 232773, 232774, 232775, 232776, 232777, 232778, 232779, 232780, 232781, 232782) c6(232783, 232784, 232785, 232786, 232787, 232788, 232789, 232790, 232791, 232792, 232793, 232794) c7(eW, cZ, os, DM, T, WL, aRx, NT, fd, bB, jh, aj, oZ, fr, ci, q, J, cd, ad, aRo, Na, aRy, hd, no, aE, rl, aT, h, Nc, K, dd, hS, rv, ae, af, vl, A, d, aW, eH, Az, eZ, r, aq, aRw, kp, eF, t, cm, bz, atb, kP, eM, nB, bE, BP, mG, aRz, ln, afd, cj, aef, asR, ro, aRA, m, y, bt, Bh, B, aRv); #c1(COL11A2) c2(XP_011512600) c3(collagen alpha-2(X1) chain isoform X1) c4(303519, 303520, 303521, 303522) c5( ) c6( ) c7(eW, cZ, os, DM, T, WL, aRx, NT, fd, bB, jh, aj, oZ, fr, ci, q, J, cd, ad, aRo, Na, aRy, hd, no, aE, rl, aT, h, Nc, K, dd, hS, rv, ae, af, vl, A, d, aW, eH, Az, eZ, r, aq, aRw, kp, eF, t, cm, bz, atb, kP, eM, nB, bE, BP, mG, aRz, ln, afd, cj, aef, asR, ro, aRA, m, y, bt, Bh, B, aRv); #c1(COL11A2) c2(XP_011512601) c3(collagen alpha-2(X1) chain isoform X2) c4(303523, 303524, 303525) c5( ) c6( ) c7(eW, cZ, os, DM, T, WL, aRx, NT, fd, bB, jh, aj, oZ, fr, ci, q, J, cd, ad, aRo, Na, aRy, hd, no, aE, rl, aT, h, Nc, K, dd, hS, rv, ae, af, vl, A, d, aW, eH, Az, eZ, r, aq, aRw, kp, eF, t, cm, bz, atb, kP, eM, nB, bE, BP, mG, aRz, ln, afd, cj, aef, asR, ro, aRA, m, y, bt, Bh, B, aRv); #c1(COL11A2) c2(XP_011512602) c3(collagen alpha-2(X1) chain isoform X3) c4(303526, 303527, 303528, 303529) c5( ) c6( ) c7(eW, cZ, os, DM, T, WL, aRx, NT, fd, bB, jh, aj, oZ, fr, ci, q, J, cd, ad, aRo, Na, aRy, hd, no, aE, rl, aT, h, Nc, K, dd, hS, rv, ae, af, vl, A, d, aW, eH, Az, eZ, r, aq, aRw, kp, eF, t, cm, bz, atb, kP, eM, nB, bE, BP, mG, aRz, ln, afd, cj, aef, asR, ro, aRA, m, y, bt, Bh, B, aRv); #c1(COL11A2) c2(XP_011512604) c3(collagen alpha-2(X1) chain isoform X5) c4(303530, 303531) c5(303532, 303533, 303534, 303535, 303536, 303537, 303538, 303539, 303540, 303541, 303542, 303543, 303544, 303545, 303546, 303547, 303548, 303549, 303550, 303551, 303552, 303553, 303554, 303555, 303556, 303557, 303558, 303559, 303560, 303561, 303562, 303563, 303564, 303565, 303566, 303567, 303568, 303569, 303570, 303571, 303572) c6(303573, 303574, 303575, 303576) c7(eW, cZ, os, DM, T, WL, aRx, NT, fd, bB, jh, aj, oZ, fr, ci, q, J, cd, ad, aRo, Na, aRy, hd, no, aE, rl, aT, h, Nc, K, dd, hS, rv, ae, af, vl, A, d, aW, eH, Az, eZ, r, aq, aRw, kp, eF, t, cm, bz, atb, kP, eM, nB, bE, BP, mG, aRz, ln, afd, cj, aef, asR, ro, aRA, m, y, bt, Bh, B, aRv); #c1(COL12A1) c2(NP_004361) c3(collagen alpha-1(XII) chain long isoform precursor) c4(145681, 145682) c5(145683, 145684, 145685, 145686, 145687, 145688, 145689, 145690, 145691, 145692, 145693, 145694, 145695, 145696, 145697, 145698, 145699, 145700, 145701, 145702, 145703, 145704, 145705) c6(145706, 145707, 145708, 145709, 145710, 145711) c7(eW, T, no, aW, rn, aRB, oj, aT, r, n, aRC, Eh, q); #c1(COL12A1) c2(NP_542376) c3(collagen alpha-1(XII) chain short isoform precursor) c4(232287) c5(232288) c6( ) c7(eW, T, no, aW, rn, aRB, oj, aT, r, n, aRC, Eh, q); #c1(COL12A1) c2(XP_011533738) c3(collagen alpha-1(XII) chain isoform X3) c4( ) c5(329845) c6( ) c7(eW, T, no, aW, rn, aRB, oj, aT, r, n, aRC, Eh, q); #c1(COL13A1) c2(NP_001123575) c3(collagen alpha-1(XIII) chain isoform 1) c4(47664, 47665, 47666, 47667, 47668, 47669, 47670) c5(47671, 47672, 47673, 47674, 47675, 47676, 47677, 47678, 47679, 47680, 47681, 47682, 47683, 47684, 47685, 47686, 47687, 47688, 47689, 47690, 47691, 47692, 47693, 47694, 47695, 47696, 47697, 47698, 47699, 47700, 47701, 47702, 47703, 47704, 47705, 47706) c6( ) c7(fc, ac, BE); #c1(COL13A1) c2(NP_542988) c3(collagen alpha-1(XIII) chain isoform 21) c4(233144, 233145) c5(233146, 233147, 233148, 233149, 233150, 233151, 233152, 233153, 233154, 233155, 233156, 233157) c6( ) c7(fc, ac, BE); #c1(COL13A1) c2(NP_542990) c3(collagen alpha-1(XIII) chain isoform 11) c4(233158, 233159) c5(233160, 233161, 233162, 233163, 233164, 233165, 233166, 233167, 233168, 233169, 233170, 233171) c6( ) c7(fc, ac, BE); #c1(COL13A1) c2(NP_542991) c3(collagen alpha-1(XIII) chain isoform 5) c4(233172, 233173, 233174) c5(233175, 233176, 233177, 233178, 233179, 233180, 233181, 233182, 233183, 233184, 233185, 233186, 233187, 233188, 233189, 233190, 233191, 233192) c6( ) c7(fc, ac, BE); #c1(COL13A1) c2(NP_542992) c3(collagen alpha-1(XIII) chain isoform 15) c4(233193) c5(233194, 233195, 233196, 233197, 233198, 233199, 233200, 233201, 233202, 233203, 233204, 233205, 233206) c6( ) c7(fc, ac, BE); #c1(COL13A1) c2(NP_542995) c3(collagen alpha-1(XIII) chain isoform 19) c4(233207) c5(233208, 233209, 233210, 233211, 233212, 233213, 233214) c6( ) c7(fc, ac, BE); #c1(COL14A1) c2(XP_005251116) c3(collagen alpha-1(XIV) chain isoform X2) c4(270818, 270819) c5(270820, 270821, 270822, 270823, 270824, 270825, 270826, 270827, 270828, 270829, 270830, 270831) c6( ) c7(rn, fr, j, g, Eg, bG); #c1(COL14A1) c2(XP_006716714) c3(collagen alpha-1(XIV) chain isoform X1) c4(290426, 290427) c5(290428, 290429, 290430, 290431, 290432, 290433, 290434, 290435, 290436, 290437, 290438, 290439) c6( ) c7(rn, fr, j, g, Eg, bG); #c1(COL15A1) c2(XP_011516516) c3(collagen alpha-1(XV) chain isoform X1) c4(309920, 309921) c5(309922, 309923, 309924, 309925, 309926, 309927, 309928, 309929, 309930, 309931, 309932, 309933) c6( ) c7(cZ, dD, dH, ea, eX, eV, eq, fr, dd, aRD, pb, bA); #c1 (COL15A1) c2(XP_011539026) c3(collagen alpha-1(XVI) chain isoform X4) c4( ) c5(337107) c6( ) c7(d); #c1(COL16A1) c2(XP_011539030) c3(collagen alpha-1(XVI) chain isoform X8) c4( ) c5(337108) c6( ) c7(d); #c1(COL16A1) c2(XP_011539032) c3(collagen alpha-1(XVI) chain isoform X10) c4( ) c5(337109, 337110, 337111, 337112, 337113, 337114, 337115, 337116, 337117, 337118, 337119, 337120) c6( ) c7(d); #c1(COL17A1) c2(NP_000485) c3(collagen alpha-1(XVII) chain) c4(9784, 9785, 9786, 9787) c5(9788, 9789, 9790, 9791, 9792, 9793, 9794, 9795, 9796, 9797, 9798, 9799, 9800, 9801, 9802, 9803, 9804, 9805, 9806, 9807, 9808, 9809, 9810, 9811, 9812, 9813, 9814, 9815, 9816, 9817, 9818, 9819, 9820) c6(9821, 9822, 9823, 9824, 9825, 9826, 9827) c7(sc, T, Ol, aGX, aRE, XM, aue, aRG, aRM, lQ, on, aRF, li, aRK, aRL, ff, aCB, ks, en, g, aRl, arJ, aRJ, aRH, ck, ay, sS, Qu, lV, uz, XO, XN, adg, arO); #c1(COL18A1) c2(NP_085059) c3(collagen alpha-1(XVIII) chain isoform 1 preproprotein) c4(219249) c5(219250, 219251, 219252, 219253, 219254, 219255) c6( ) c7(gh, T, aC, al, fd, bB, ci, aR, x, aE, eL, DA, up, JI, aW, dH, lh, ark, zC, g, as, eF, cm, qJ, eM, nB, iA, cj, k, m, lS, n, fr, nA, zP, eh, eW, yn, EI, BI, Dr, Xe, dL, hB, cY, wC, aq, CO, W, dB, Vy, Be, at, B, bL, ul, aRN, fZ, aA, wo, hy, v, j, q, eA, aT, KU, ae, af, dl, ne, jf, qw, s, oV, r, en, t, f, iK, ck, ov, bt, ha, jh, Fb, ad, dP, av, Rq, DI, d, BN, ff, eH, aF, Ed, iV, iR, bz, LB, es, bF, bE, GF, y, vt, X); #c1(COL18A1) c2(NP_569711) c3(collagen alpha-1(XVIII) chain isoform 3 preproprotein) c4(233792, 233793) c5(233794, 233795, 233796, 233797, 233798, 233799, 233800, 233801, 233802, 233803, 233804, 233805) c6( ) c7(gh, T, aC, al, fd, bB, ci, aR, x, aE, eL, DA, up, JI, aW, dH, Ih, ark, zC, g, as, eF, cm, qJ, eM, nB, iA, cj, k, m, lS, n, fr, nA, zP, eh, eW, yn, EI, BI, Dr, Xe, dL, hB, cY, wC, aq, CO, W, dB, Vy, Be, at, B, bL, ul, aRN, fZ, aA, wo, hy, v, j, q, eA, aT, KU, ae, af, dl, ne, jf, qw, s, oV, r, en, t, f, ik, ck, ov, bt, ha, jh, Fb, ad, dP, av, Rq, DI, d, BN, ff, eH, aF, Ed, iV, jR, bz, LB, es, bF, bE, GF, y, vt, X); #c1(COL18A1) c2(NP_569712) c3(collagen alpha-1(XVIII) chain isoform 2 preproprotein) c4(233806, 233807) c5(233808, 233809, 233810, 233811, 233812, 233813, 233814, 233815, 233816, 233817, 233818, 233819) c6( ) c7(gh, T, aC, al, fd, bB, ci, aR, x, aE, eL, DA, up, JI, aW, dH, lh, ark, zC, g, as, eF, cm, qJ, eM, nB, iA, cj, k, m, lS, n, fr, nA, zP, eh, eW, yn, EI, BI, Dr, Xe, dL, hB, cY, wC, aq, CO, W, dB, Vy, Be, at, B, bL, ul, aRN, fZ, aA, wo, hy, v, j, q, eA, aT, KU, ae, af, dl, ne, jf, qw, s, oV, r, en, t, f, ik, ck, ov, bt, ha, jh, Fb, ad, dP, av, Rq, DI, d, BN, ff, eH, aF, Ed, iV, iR, bz, LB, es, bF, bE, GF, y, vt, X); #c1(COL1A1) c2(NP_000079) c3(collagen alpha-1(l) chain preproprotein) c4(1271) c5( ) c6( ) c7(bM, T, asg, fR, al, lL, fd, lR, ci, XW, ws, rw, rn, ari, HR, bc, aRY, eG, Rh, up, pr, aW, aRO, aRW, aRS, hJ, eF, uC, aSb, iP, iA, lq, uo, pw, lS, gU, pG, vl, sQ, aRX, aw, n, zw, aSa, rl, aaj, aRd, aRP, asl, fQ, aq, op, lX, hk, Be, at, uz, ut, B, rp, tf, fG, cz, v, j, Eh, aT, rD, zp, lM, eC, af, bA, aRR, bQ, pb, aDQ, ck, BU, aGo, aRZ, ro, sY, li, ah, jE, azk, wD, hX, nH, sN, lQ, ul, lU, aRV, Fz, eH, LB, es, yE, aRU, aRT); #c1(COL1A1) c2(XP_005257115) c3 (collagen alpha-1(I) chain isoform X2) c4( ) c5(274692, 274693) c6( ) c7(bM, T, asg, fR, al, lL, fd, lR, ci, XW, ws, rw, rn, ari, HR, bc, aRY, eG, Rh, up, pr, aW, aRO, aRW, aRS, hJ, eF, uC, aSb, iP, iA, lq, uo, pw, lS, gU, pG, vl, sQ, aRX, aw, n, zw, aSa, rl, aaj, aRd, aRP, asL, fQ, aq, op, lX, hk, Be, at, uz, ut, B, rp, tf, fG, cz, v, j, Eh, aT, rD, zp, lM, eC, af, bA, aRR, bQ, pb, aDQ, ck, BU, aGo, aRZ, ro, sY, li, ah, jE, azk, wD, hX, nH, sN, lQ, uQ, lU, aRV, Fz, eH, LB, es, yE, aRU, aRT); #c1(COL1A1) c2(XP_005257116) c3(collagen alpha-1(1) chain isoform X3) c4( ) c5(274694, 274695, 274696, 274697, 274698, 274699, 274700, 274701, 274702) c6( ) c7(bM, T, asg, fR, al, lL, fd, lR, ci, XW, ws, rw, rn, ari, HR, bc, aRY, eG, Rh, up, pr, aW, aRO, aRW, aRS, hJ, eF, uC, aSb, iP, iA, lq, uo, pw, lS, gU, pG, vl, sQ, aRX, aw, n, zw, aSa, rl, aaj, aRQ, aRP, asL, fQ, aq, op, lX, hk, Be, at, uz, ut, B, rp, tf, fG, cz, v, j, Eh, aT, rD, zp, lM, eC, af, bA, aRR, bQ, pb, aDQ, ck, BU, aGo, aRZ, ro, sY, li, ah, jE, azk, wD, hX, nH, sN, lQ, ul, lU, aRV, Fz, eH, LB, es, yE, aRU, aRT); #c1(COL1A2) c2(NP_000080) c3(collagen alpha-2(l) chain precursor) c4(1272, 1273) c5(1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378) c6(1379, 1380, 1381, 1382, 1383) c7(sY, T, vl, eR, sQ, FG, fG, e, al, lL, vr, lR, aml, azk, Eh, DZ, aSe, awa, aSd, rl, aAh, aT, vc, dL, aSf, lQ, zp, bl, pj, aRP, eL, aRY, ao, bA, jf, lU, Rh, aPH, up, ff, Fz, fQ, aRW, arq, en, uC, pb, dr, arv, sS, ac, aSc, yE, bm, uo, at, ut, ajY, gT, B, us); #c1(COL20A1) c2(NP_065933) c3(collagen alpha-(XX) chain precursor) c4(206311) c5(206312, 206313, 206314, 206315, 206316, 206317, 206318, 206319) c6(206320, 206321) c7(aW, aT); #c1(COL20A1) c2(XP_011527239) c3(collagen alpha-1(XX) chain isoform X2) c4( ) c5(321792) c6( ) c7(aW, aT); #c1(COL20A1) c2(XP_011527241) c3(collagen alpha-1(XX) chain isoform X4) c4(321793) c5(321794, 321795, 321796, 321797, 321798, 321799, 321800, 321801) c6(321802, 321803) c7(aW, aT); #c1(COL20A1) c2(XP_011527242) c3(collagen alpha-1(XX) chain isoform X5) c4(321804) c5(321805, 321806, 321807, 321808, 321809, 321810, 321811, 321812, 321813, 321814, 321815, 321816, 321817, 321818) c6(321819, 321820, 321821, 321822) c7(aW, aT); #c1(COL21A1) c2(NP_110447) c3(collagen alpha-1(XX1) chain precursor) c4(220405, 220406, 220407, 220408, 220409) c5(220410, 220411, 220412, 220413, 220414, 220415, 220416, 220417, 220418, 220419, 220420, 220421, 220422, 220423, 220424, 220425, 220426, 220427, 220428, 220429, 220430, 220431, 220432) c6( ) c7(ey, cu, bK, ac, ad); #c1(COL21A1) c2(XP_006715286) c3(collagen alpha-1(XX1) chain isoform X2) c4( ) c5(289480, 289481, 289482, 289483, 289484, 289485) c6( ) c7(ey, cu, bk, ac, ad); #c1(COL21A1) c2(XP_006715288) c3(collagen alpha-1(XX1) chain isoform X3) c4( ) c5(289486, 289487, 289488, 289489, 289490) c6( ) c7(ey, cu, bK, ac, ad); #c1(COL22A1) c2(NP_690848) c3(collagen alpha-1(XXII) chain precursor) c4(244959) c5(244960) c6( ) c7(ft, ac, aSg, e, xs, EQ, Ea, an, af); #c1(COL22A1) c2(XP_011515185) c3(collagen alpha-1(XXII) chain isoform X1) c4(308025) c5( ) c6( ) c7(ft, ac, aSg, e, xs, EQ, Ea, an, af); #c1(COL22A1) c2(XP_011515187) c3(collagen alpha-1(XXII) chain isoform X3) c4(308026) c5( ) c6( ) c7(ft, ac, aSg, e, xs, EQ, Ea, an, af); #c1(COL22A1) c2(XP_011515190) c3(collagen alpha-1(XXII) chain isoform X6) c4( ) c5(308027, 308028, 308029, 308030, 308031, 308032, 308033, 308034, 308035, 308036, 308037, 308038) c6( ) c7(ft, ac, aSg, e, xs, EQ, Ea, an, af); #c1(COL22A1) c2(XP_011515192) c3(collagen alpha-1(XXII) chain isoform X8) c4( ) c5(308039) c6( ) c7(ft, ac, aSg, e, xs, EQ, Ea, an, af); #c1(COL22A1) c2(XP_011515193) c3(collagen alpha-1(XXII) chain isoform X9) c4( ) c5(308040, 308041, 308042, 308043, 308044, 308045) c6( ) c7(ft, ac, aSg, e, xs, EQ, Ea, an, af); #c1(COL23A1) c2(NP_775736) c3(collagen alpha-1(XXIII) chain) c4(250118, 250119) c5(250120, 250121, 250122, 250123, 250124, 250125) c6( ) c7(dy, ac); #c1(COL23A1) c2(XP_011532994) c3(collagen alpha-1(XXIII) chain isoform X9) c4( ) c5(329230, 329231, 329232, 329233, 329234, 329235, 329236) c6( ) c7(dy, ac); #c1(COL24A1) c2(XP_011539493) c3(collagen alpha-1(XXIV) chain isoform X2) c4( ) c5(337692, 337693, 337694, 337695, 337696, 337697) c6( ) c7(bA); #c1(COL25Al) c2(NP_001243003) c3(collagen alpha-1(XXV) chain isoform 3) c4(89528) c5(89529, 89530, 89531, 89532, 89533, 89534) c6( ) c7(uk, L, B, aq, f, H, af, FT, hq); #c1(COL25Al) c2(NP_115907) c3(collagen alpha-(XXV) chain isoform 2) c4(224613) c5(224614, 224615, 224616, 224617, 224618, 224619, 224620, 224621, 224622, 224623, 224624, 224625) c6( ) c7(uk, L, B, aq, f, H, af, FT, hq); #c1(COL25A1) c2(NP_942014) c3(collagen alpha-1(XXV) chain isoform 1) c4(260416) c5(260417, 260418, 260419, 260420, 260421, 260422, 260423, 260424, 260425, 260426, 260427, 260428) c6( ) c7(uk, L, B, aq, f, H, af, FT, hq); #c1(COL25A1) c2(XP_011530646) c3(collagen alpha-1(XXV) chain isoform X10) c4(326728, 326729) c5(326730, 326731, 326732, 326733, 326734, 326735) c6( ) c7(uk, L, B, aq, f, H, af, FT, hq); #c1(COL25Al) c2(XP_011530651) c3(collagen alpha-1(XXV) chain isoform X15) c4(326736) c5( ) c6( ) c7(uk, L, B, aq, f, H, af, FT, hq); #c1(COL25A1) c2(XP_011530652) c3(collagen alpha-1(XXV) chain isoform X16) c4(326737) c5( ) c6( ) c7(uk, L, B, aq, f, H, af, FT, hq); #c1(COL25Al) c2(XP_011530655) c3(collagen alpha-1(XXV) chain isoform X19) c4(326738) c5( ) c6( ) c7(uk, L, B, aq, f, H, af, FT, hq); #c1(COL25Al) c2(XP_011530661) c3(collagen alpha-1(XXV) chain isoform X25) c4( ) c5(326739, 326740, 326741, 326742, 326743, 326744) c6( ) c7(uk, L, B, aq, f, H, af, FT, hq); #c1(COL26A1) c2(NP_001265492) c3(collagen alpha-1(XXVI) chain isoform 1 precursor) c4(98704) c5(98705, 98706, 98707, 98708, 98709, 98710, 98711, 98712) c6(98713, 98714) c7(bM, aw); #c1(COL26A1) c2(NP_597714) c3(collagen alpha-1(XXVI) chain isoform 2 precursor) c4( ) c5(235087, 235088) c6( ) c7(bM, aw); #c1(COL27A1) c2(NP_116277) c3(collagen alpha-1(XXVII) chain preproprotein) c4(226052, 226053) c5(226054, 226055, 226056, 226057, 226058, 226059, 226060, 226061, 226062, 226063, 226064, 226065) c6( ) c7(ro, ru); #c1(COL27A1) c2(XP_006717371) c3(collagen alpha-1(XXVII) chain isoform X4) c4(291031) c5(291032, 291033, 291034, 291035, 291036, 291037) c6( ) c7(ro, ru); #c1(COL27A1) c2(XP_011517447) c3(collagen alpha-1(XXVII) chain isoform X10) c4( ) c5(311157) c6( ) c7(ro, ru); #c1(COL28Al) c2(XP_011513662) c3(collagen alpha-1(XXVIII) chain isoform X1) c4(305964, 305965) c5(305966, 305967, 305968, 305969, 305970, 305971) c6( ) c7(xa, bk); #c1(COL28A1) c2(XP_011513663) c3(collagen alpha-1(XXVIII) chain isoform X2) c4(305972) c5( ) c6( ) c7(xa, bk); #c1(COL28A1) c2(XP_011513664) c3(collagen alpha-1(XXVIII) chain isoform X3) c4( ) c5(305973, 305974, 305975, 305976, 305977, 305978, 305979) c6( ) c7(xa, bK); #c1(COL28A1) c2(XP_011513665) c3(collagen alpha-1(XXVIII) chain isoform X4) c4(305980) c5(305981, 305982, 305983, 305984, 305985, 305986) c6( ) c7(xa, bK); #c1(COL28A1) c2(XP_011513668) c3(collagen alpha-1(XXVIII) chain isoform X7) c4( ) c5(305987, 305988, 305989, 305990, 305991, 305992, 305993) c6( ) c7(xa, bK); #c1(COL2A1) c2(NP_001835) c3(collagen alpha-1(II) chain isoform 1 precursor) c4(121562) c5( ) c6( ) c7(T, aSz, fR, rV, aSy, bB, aSl, aSn, Na, aRy, HV, rw, Fo, aRY, aSj, alk, lh, Az, aSt, aRq, rq, BP, apy, fy, aSs, lc, aSq, vl, n, aRt, aSv, ai, ajO, rl, ey, Wr, rk, Dg, aSr, aSm, vU, ln, Be, aSx, lA, Tr, aSh, Eh, q, aRo, aSo, ld, rD, Nc, an, aSi, rv, jr, rh, Y, ev, fO, r, rs, Ci, aDA, vk, vK, mG, HW, aSu, aDR, aSk, gT, kh, ade, aSw, gb, Dj, ji, aFl, aGS, zr, bE, ag, aSp, y, X, aCR); #c1(COL2A1) c2(NP_149162) c3(collagen alpha-1(Il) chain isoform 2 precursor) c4(227430, 227431) c5( ) c6( ) c7(T, aSz, fR, rV, aSy, bB, aSl, aSn, Na, aRy, HV, rw, Fo, aRY, aSj, alk, lh, Az, aSt, aRq, rq, BP, apy, fy, aSs, lc, aSq, vl, n, aRt, aSv, ai, ajO, rl, ey, Wr, rk, Dg, aSr, aSm, vU, ln, Be, aSx, lA, Tr, aSh, Eh, q, aRo, aSo, ld, rD, Nc, an, aSi, rv, jr, rh, Y, ev, fO, r, rs, Ci, aDQ, vk, vK, mG, HW, aSu, aDR, aSk, gT, kh, ade, aSw, gb, Dj, ji, aFl, aGS, zr, bE, ag, aSp, y, X, aCR); #c1(COL2A1) c2(XP_011536236) c3(collagen alpha-1(II) chain isoform X2) c4( ) c5(333558) c6( ) c7(T, aSz, fR, rV, aSy, bB, aSl, aSn, Na, aRy, HV, rw, Fo, aRY, aSj, alk, lh, Az, aSt, aRq, rq, BP, apy, fy, aSs, lc, aSq, vl, n, aRt, aSv, ai, ajO, rl, ey, Wr, rk, Dg, aSr, aSm, vU, ln, Be, aSx, lA, Tr, aSh, Eh, q, aRo, aSo, ld, rD, Nc, an, aSi, rv, jr, rh, Y, ev, fO, r, rs, Ci, aDO, vk, vK, mG, HW, aSu, aDR, aSk, gT, kh, ade, aSw, gb, Dj, ji, aFl, aGS, zr, bE, ag, aSp, y, X, aCR); #c1(COL2A1) c2(XP_011536237) c3(collagen alpha-l(Il) chain isoform X3) c4(333559) c5(333560, 333561, 333562, 333563, 333564, 333565, 333566, 333567, 333568, 333569, 333570, 333571, 333572, 333573, 333574, 333575, 333576, 333577, 333578, 333579) c6( ) c7(T, aSz, fR, rV, aSy, bB, aSl, aSn, Na, aRy, HV, rw, Fo, aRY, aSj, alk, lh, Az, aSt, aRq, rq, BP, apy, fy, aSs, lc, aSq, vl, n, aRt, aSv, ai, aj0, rl, ey, Wr, rk, Dg, aSr, aSm, vU, ln, Be, aSx, lA, Tr, aSh, Eh, q, aRo, aSo, ld, rD, Nc, an, aSi, rv, jr, rh, Y, ev, fO, r, rs, Ci, aDO, vk, vK, mG, HW, aSu, aDR, aSk, gT, kh, ade, aSw, gb, Dj, ji, aFl, aGS, zr, bE, ag, aSp, y, X, aCR); #c1(COL3A1) c2(NP_000081) c3(collagen alpha-l(III) chain preproprotein) c4(1384, 1385) c5(1386, 1387, 1388, 1389, 1390, 1391, 1392, 1393, 1394, 1395, 1396, 1397, 1398, 1399, 1400) c6( ) c7(aSB, aSH, sY, vl, fR, sQ, lP, eR, wJ, aw, al, Av, lL, aSA, cz, n, aSE, dG, j, jE, Mi, vp, Cd, Eh, aSG, kL, cc, ws, rl, le, aSF, jy, HR, K, lQ, zp, lz, eN, af, ul, aSC, bf, cH, Fz, hl, fc, fQ, Au, aSD, uC, dr, op, LB, ac, FB, bm, uo, ut, rx, aS, tr, be, ei, cE, JO, us); #c1(COL4A1) c2(NP_001290039) c3(collagen alpha-l(IV) chain isoform 2 precursor) c4(114948) c5(114949, 114950, 114951, 114952, 114953, 114954, 114955, 114956, 114957) c6(114958, 114959) c7(lp, sY, Ez, T, dD, aib, aw, cz, j, HS, ah, aSL, eq, ld, eA, ad, aJr, aSM, sd, oj, aT, vw, us, K, an, he, aSl, ae, vl, rz, d, cH, aW, Y, aSK, ack, aSJ, S, uC, D, aLO, ac, uz, aS, dy, lM, Ex, lf); #c1(COL4A1) c2(NP_001836) c3(collagen alpha-l(IV) chain isoform 1 preproprotein) c4(121563, 121564, 121565, 121566) c5(121567, 121568, 121569, 121570, 121571, 121572, 121573, 121574, 121575, 121576, 121577, 121578, 121579, 121580, 121581, 121582, 121583, 121584, 121585, 121586, 121587, 121588, 121589, 121590) c6(121591, 121592, 121593, 121594, 121595, 121596) c7(lp, sY, Ez, T, dD, aib, aw, cz, j, HS, ah, aSL, eq, ld, eA, ad, aJr, aSM, sd, oj, aT, vw, us, K, an, he, aSl, ae, vl, rz, d, cH, aW, Y, aSK, ack, aSJ, S, uC, D, aLO, ac, uz, aS, dy, lM, Ex, lf); #c1(COL4A1) c2(XP_011519350) c3(collagen alpha-1(IV) chain isoform X1) c4( ) c5(314008, 314009, 314010) c6( ) c7(lp, sY, Ez, T, dD, aib, aw, cz, j, HS, ah, aSL, eq, ld, eA, ad, aJr, aSM, sd, oj, aT, vw, us, K, an, he, aSl, ae, vl, rz, d, cH, aW, Y, aSK, ack, aSJ, S, uC, D, aLO, ac, uz, aS, dy, lM, Ex, lf); #c1(COL4A2) c2(NP_001837) c3(collagen alpha-2(IV) chain preproprotein) c4(121597, 121598, 121599) c5(121600, 121601, 121602, 121603, 121604, 121605, 121606, 121607, 121608, 121609, 121610, 121611, 121612, 121613, 121614, 121615, 121616, 121617, 121618, 121619, 121620, (21621) c6( ) c7(lp, sY, T, e, n, j, ci, uE, ah, ld, ad, ft, sd, aE, oj, aT, ey, K, he, uM, aW, Y, aSK, uC, bz, fq, aLO, ac, aSN, dy); #c1(COL4A3) c2(NP_000082) c3(collagen alpha-3(IV) chain precursor) c4(1401, 1402) c5( ) c6( ) c7(bM, sY, eR, aSP, sw, ah, HS, uE, fr, aY, aSR, eA, ad, fU, aSU, ab, vw, K, aSS, ae, aST, UM, DA, acF, Y, ff, and, aSO, en, uC, sk, aLO, eM, iA, bE, hc, fP, PV, at, dy, Hb, ME, aSQ, aU, B, jr); #c1(COL4A3) c2(XP_005246333) c3(collagen alpha-3(IV) chain isoform X1) c4(267051, 267052, 267053) c5( ) c6( ) c7(bM, sY, eR, aSP, sw, ah, HS, uE, fr, aY, aSR, eA, ad, fU, aSU, ab, vw, K, aSS, ae, aST, UM, DA, acF, Y, ff, and, aSO, en, uC, sk, aLO, eM, iA, bE, hc, fP, PV, at, dy, Hb, ME, aSQ, aU, B, jr); #c1(COL4A3) c2(XP_005246334) c3(collagen alpha-3(IV) chain isoform X2) c4( ) c5(267054) c6( ) c7(bM, sY, eR, aSP, sw, ah, HS, uE, fr, aY, aSR, eA, ad, fU, aSU, ab, vw, K, aSS, ae, aST, uM, DA, acF, Y, ff, and, aSO, en, uC, sk, aLO, eM, iA, bE, hc, fP, PV, at, dy, Hb, ME, aSQ, aU, B, jr); #c1(COL4A3) c2(XP_005246337) c3(collagen alpha-3(IV) chain isoform X6) c4( ) c5(267055, 267056, 267057, 267058, 267059, 267060, 267061, 267062, 267063, 267064, 267065, 267066, 267067, 267068, 267069, 267070, 267071, 267072, 267073, 267074, 267075, 267076, 267077, 267078) c6( ) c7(bM, sY, eR, aSP, sw, ah, HS, uE, fr, aY, aSR, eA, ad, fU, aSU, ab, vw, K, aSS, ae, aST, UM, DA, acF, Y, ff, anQ, aSO, en, uC, sk, aLO, eM, iA, bE, hc, fP, PV, at, dy, Hb, ME, aSQ, all, B, jr); #c1(COL4A3) c2(XP_011508858) c3(collagen alpha-3(IV) chain isoform X4) c4( ) c5(299450, 299451, 299452, 299453, 299454, 299455, 299456) c6( ) c7(bM, sY, eR, aSP, sw, ah, HS, uE, fr, aY, aSR, eA, ad, fU, aSU, ab, vw, K, aSS, ae, aST, uM, DA, acF, Y, ff, and, aSO, en, uC, sk, aLO, eM, iA, bE, hc, fP, PV, at, dy, Hb, ME, aSQ, aU, B, jr); #c1(COL4A3BP) c2(NP_001123577) c3(collagen type IV alpha-3-binding protein isoform 3) c4(47707, 47708, 47709) c5(47710, 47711, 47712, 47713, 47714, 47715, 47716, 47717, 47718, 47719, 47720, 47721, 47722) c6( ) c7(T, af, eM, aW, hc, Y, eZ, aT, ME, m, fl, t, H, mC, L); #c1(COL4A3BP) c2(NP_005704) c3(collagen type IV alpha-3-binding protein isoform 1) c4(158665) c5( ) c6( ) c7(T, af, eM, aW, hc, Y, eZ, aT, ME, m, fl, t, H, mC, L); #c1(COL4A3BP) c2(NP_112729) c3(collagen type IV alpha-3-binding protein isoform 2) c4(221547, 221548, 221549) c5(221550, 221551, 221552, 221553, 221554, 221555, 221556, 221557, 221558, 221559, 221560, 221561, 221562, 221563, 221564, 221565, 221566, 221567, 221568) c6( ) c7(T, af, eM, aW, hc, Y, eZ, aT, ME, m, fl, t, H, mC, L); #c1(COL4A4) c2(XP_005246338) c3(collagen alpha-4(IV) chain isoform X1) c4(267079, 267080) c5(267081) c6( ) c7(sY, eR, dk, HS, aSV, aY, aSR, fU, aSU, ab, vw, aSS, UM, aST, acF, uC, sk, aLO, mG, fP, dy, aSQ, jr); #c1(COL4A4) c2(XP_011508873) c3(collagen alpha-4(IV) chain isoform X15) c4( ) c5(299457, 299458, 299459, 299460, 299461, 299462, 299463, 299464, 299465, 299466, 299467, 299468) c6( ) c7(sY, eR, dk, HS, aSV, aY, aSR, fU, aSU, ab, vw, aSS, uM, aST, acF, uC, sk, aLO, mG, fP, dy, aSQ, jr); #c1(COL4A4) c2(XP_011508874) c3(collagen alpha-4(IV) chain isoform X16) c4( ) c5(299469, 299470) c6( ) c7(sY, eR, dk, HS, aSV, aY, aSR, fU, aSU, ab, vw, aSS, uM, aST, acF, uC, sk, aLO, mG, fP, dy, aSQ, jr); #c1 (COL4A5) c2(NP_000486) c3(collagen alpha-5(IV) chain isoform 1 precursor) c4(9828) c5( ) c6( ) c7(bM, sY, T, kh, eR, dM, ha, ah, HS, nA, uE, aY, aSR, rc, Zp, lS, bW, nd, aT, ey, aSX, UM, acF, lR, aW, nf, g, aTa, aRC, uC, bz, aTb, dr, op, aSZ, LB, sk, aLO, Bv, afZ, hc, mG, vA, agl, aSW, dy, vF, aSQ, aSY, iu, jr); #c1(COL4A5) c2(XP_005262127) c3(collagen alpha-5(IV) chain isoform X2) c4( ) c5(277751) c6( ) c7(bM, sY, T, kh, eR, dM, ha, ah, HS, nA, uE, aY, aSR, rc, Zp, lS, bW, nd, aT, ey, aSX, UM, acF, lR, aW, nf, g, aTa, aRC, uC, bz, aTb, dr, op, aSZ, LB, sk, aLO, Bv, afZ, hc, mG, vA, agl, aSW, dy, vF, aSQ, aSY, iu, jr); #c1(COL4A5) c2(XP_005262129) c3(collagen alpha-5(IV) chain isoform X5) c4( ) c5(277752) c6( ) c7(bM, sY, T, kh, eR, dM, ha, ah, HS, nA, uE, aY, aSR, rc, Zp, lS, bW, nd, aT, ey, aSX, uM, acF, lR, aW, nf, g, aTa, aRC, uC, bz, aTb, dr, op, aSZ, LB, sk, aLO, Bv, afZ, hc, mG, vA, agl, aSW, dy, vF, aSQ, aSY, iu, jr); #c1(COL4A5) c2(XP_006724679) c3(collagen alpha-5(IV) chain isoform X1) c4(296251, 296252) c5(296253) c6( ) c7(bM, sY, T, kh, eR, dM, ha, ah, HS, nA, uE, aY, aSR, rc, Zp, lS, bW, nd, aT, ey, aSX, UM, acF, lR, aW, nf, g, aTa, aRC, uC, bz, aTb, dr, op, aSZ, LB, sk, aLO, Bv, afZ, hc, mG, vA, agl, aSW, dy, vF, aSQ, aSY, iu, jr); #c1(COL4A5) c2(XP_011529153) c3(collagen alpha-5(IV) chain isoform X6) c4( ) c5(324498, 324499, 324500, 324501, 324502, 324503, 324504, 324505, 324506, 324507, 324508, 324509, 324510, 324511, 324512, 324513, 324514) c6( ) c7(bM, sY, T, kh, eR, dM, ha, ah, HS, nA, uE, aY, aSR, rc, Zp, lS, bW, nd, aT, ey, aSX, UM, acF, lR, aW, nf, g, aTa, aRC, uC, bz, aTb, dr, op, aSZ, LB, sk, aLO, Bv, afZ, hc, mG, vA, agl, aSW, dy, vF, aSQ, aSY, iu, jr); #c1(COL4A6) c2(NP_001274688) c3(collagen alpha-6(IV) chain isoform 4 precursor) c4(106660, 106661) c5( ) c6( ) c7(sY, T, alc, s, op, bE, Zp, vA, ff, bB, dy, aTd, HS, en, g, aY, uC, Bh, aTb, dr, lS); #c1(COL4A6) c2(NP_001274689) c3(collagen alpha-6(IV) chain isoform 5 precursor) c4(106662) c5(106663, 106664, 106665, 106666, 106667, 106668, 106669, 106670, 106671, 106672, 106673, 106674) c6( ) c7(sY, T, alc, s, op, bE, Zp, vA, ff, bB, dy, aTd, HS, en, g, aY, uC, Bh, aTb, dr, lS); #c1(COL4A6) c2(NP_001838) c3(collagen alpha-6(IV) chain isoform A precursor) c4(121622) c5( ) c6( ) c7(sY, T, aTc, s, op, bE, Zp, vA, ff, bB, dy, aTd, HS, en, g, aY, uC, Bh, aTb, dr, lS); #c1(COL4A6) c2(NP_378667) c3(collagen alpha-6(IV) chain isoform B precursor) c4(228938, 228939, 228940) c5( ) c6( ) c7(sY, T, alc, s, op, bE, Zp, vA, ff, bB, dy, aTd, HS, en, g, aY, uC, Bh, aTb, dr, lS); #c1(COL4A6) c2(XP_011529154) c3(collagen alpha-6(IV) chain isoform X2) c4( ) c5(324515, 324516, 324517, 324518, 324519, 324520) c6( ) c7(sY, T, alc, s, op, bE, Zp, vA, ff, bB, dy, aTd, HS, en, g, aY, uC, Bh, aTb, dr, lS); #c1(COL4A6) c2(XP_011529155) c3(collagen alpha-6(IV) chain isoform X3) c4( ) c5(324521, 324522, 324523, 324524, 324525, 324526, 324527, 324528, 324529, 324530, 324531, 324532, 324533, 324534, 324535, 324536, 324537, 324538, 324539, 324540, 324541, 324542, 324543, 324544, 324545) c6( ) c7(sY, T, aTc, s, op, bE, Zp, vA, ff, bB, dy, aTd, HS, en, g, aY, uC, Bh, aTb, dr, lS); #c1 (COL4A6) c2(XP_011529156) c3(collagen alpha-6(IV) chain isoform X4) c4( ) c5(324546) c6( ) c7(sY, T, alc, s, op, bE, Zp, vA, ff, bB, dy, aTd, HS, en, g, aY, uC, Bh, aTb, dr, lS); #c1(COL5A1) c2(NP_000084) c3(collagen alpha-1(V) chain isoform 1 preproprotein) c4(1403) c5(1404, 1405, 1406, 1407, 1408, 1409, 1410, 1411, 1412, 1413) c6(1414, 1415) c7(sY, pG, fR, sQ, lL, ci, zP, Eh, pn, ip, HV, aTf, Cj, rl, ab, lQ, aDp, ant, zQ, la, alg, aTh, uC, dr, uo, ut, ro, aTe); #c1(COL5A1) c2(NP_001265003) c3(collagen alpha-1(V) chain isoform 2 preproprotein) c4(97691, 97692) c5(97693, 97694, 97695, 97696, 97697, 97698, 97699, 97700, 97701, 97702, 97703, 97704, 97705, 97706, 97707, 97708) c6(97709, 97710, 97711, 97712) c7(sY, pG, fR, sQ, lL, ci, zP, Eh, pn, ip, HV, aTf, Cj, rl, ab, lQ, aDp, ant, zQ, la, aTg, aTh, uC, dr, uo, ut, ro, ale); #c1(COL5A2) c2(NP_000384) c3(collagen alpha-2(V) chain preproprotein) c4(8357, 8358) c5(8359, 8360, 8361, 8362, 8363, 8364, 8365, 8366, 8367, 8368, 8369, 8370) c6( ) c7(sY, T, HV, uC, fR, sQ, rl, Eh, e, lL, r, uo, aTg, ut, aaQ, j, lQ, ei, mo, ako, q); #c1(COL5A2) c2(XP_011508875) c3(collagen alpha-2(V) chain isoform X1) c4( ) c5(299471, 299472, 299473) c6( ) c7(sY, T, HV, uC, fR, sQ, rl, Eh, e, lL, r, uo, alg, ut, aaQ, j, lQ, ei, mo, ako, q); #c1(COL5A3) c2(NP_056534) c3(collagen alpha-3(V) chain preproprotein) c4(187987) c5(187988, 187989, 187990, 187991, 187992, 187993) c6( ) c7(Eh); #c1(COL5A3) c2(XP_011526344) c3(collagen alpha-3(V) chain isoform X1) c4( ) c5(320648) c6( ) c7(Eh); #c1(COL6A1) c2(NP_001839) c3(collagen alpha-1(VI) chain precursor) c4(121623, 121624, 121625) c5(121626, 121627, 121628, 121629, 121630, 121631, 121632, 121633, 121634, 121635, 121636, 121637, 121638, 121639, 121640, 121641, 121642, 121643) c6( ) c7(av, mR, Wn, aBq, xs, oj, bD, arw, asR, j, v, as, akb, f, aTi, qW, af); #c1 (COL6A2) c2(NP_478054) c3(collagen alpha-2(VI) chain isoform 2C2a precursor) c4(230820) c5(230821, 230822, 230823, 230824, 230825, 230826, 230827, 230828, 230829, 230830) c6( ) c7(ly, aTj, Wn, eE, xs, aTk, oj, mg, fQ, yU, arw, yZ, bD, f, aTi, af); #c1(COL6A2) c2(NP_478055) c3(collagen alpha-2(VI) chain isoform 2C2a′ precursor) c4(230831, 230832) c5(230833, 230834, 230835, 230836, 230837, 230838, 230839, 230840, 230841, 230842, 230843, 230844, 230845, 230846, 230847, 230848) c6( ) c7(ly, aTj, Wn, eE, xs, aTk, oj, mg, fQ, yU, arw, yZ, bD, f, aTi, af); #c1(COL6A2) c2(XP_011527754) c3(collagen alpha-2(VI) chain isoform X1) c4(322735) c5(322736, 322737, 322738, 322739, 322740, 322741) c6( ) c7(ly, aTj, Wn, eE, xs, aTk, oj, mg, fQ, yU, arw, yZ, bD, f, aTi, af); #c1(COL6A3) c2(NP_004360) c3(collagen alpha-3(VI) chain isoform 1 precursor) c4(145669, 145670) c5(145671, 145672, 145673, 145674, 145675, 145676, 145677, 145678) c6(145679, 145680) c7(VC, ly, T, ac, aE, Wn, Y, eE, xs, oj, arw, aTl, v, kp, zu, f, ali, ad, aTm); #c1(COL6A3) c2(NP_476505) c3(collagen alpha-3(VI) chain isoform 2 precursor) c4(230623) c5(230624, 230625, 230626, 230627, 230628, 230629, 230630, 230631) c6(230632, 230633) c7(VC, ly, T, ac, aE, Wn, Y, eE, xs, oj, arw, aTl, v, kp, zu, f, ali, ad, aTm); #c1(COL6A3) c2(NP_476506) c3(collagen alpha-3(VI) chain isoform 3 precursor) c4(230634) c5(230635, 230636, 230637, 230638, 230639, 230640) c6( ) c7(VC, ly, T, ac, aE, Wn, Y, eE, xs, oj, arw, aTl, v, kp, zu, f, ali, ad, aTm); #c1(COL6A3) c2(NP_476507) c3(collagen alpha-3(VI) chain isoform 4 precursor) c4(230641) c5(230642, 230643, 230644, 230645, 230646, 230647) c6( ) c7(VC, ly, T, ac, aE, Wn, Y, eE, xs, oj, arw, aTl, v, kp, zu, f, ali, ad, aTm); #c1(COL6A3) c2(NP_476508) c3(collagen alpha-3(VI) chain isoform 5 precursor) c4(230648, 230649) c5(230650, 230651, 230652, 230653, 230654, 230655, 230656, 230657, 230658, 230659, 230660, 230661, 230662, 230663, 230664, 230665, 230666, 230667, 230668, 230669, 230670, 230671) c6(230672, 230673, 230674, 230675, 230676, 230677) c7(VC, ly, T, ac, aE, Wn, Y, eE, xs, oj, arw, aTl, v, kp, zu, f, aTi, ad, aTm); #c1(COL6A3) c2(XP_005246122) c3(collagen alpha-3(VI) chain isoform X3) c4(266938) c5(266939, 266940, 266941, 266942, 266943, 266944, 266945, 266946) c6(266947, 266948) c7(VC, ly, T, ac, aE, Wn, Y, eE, xs, oj, arw, aTl, v, kp, zu, f, aTi, ad, aTm); #c1(COL6A3) c2(XP_011508877) c3(collagen alpha-3(VI) chain isoform X5) c4( ) c5(299474) c6( ) c7(VC, ly, T, ac, aE, Wn, Y, eE, xs, oj, arw, aTl, v, kp, zu, f, aTi, ad, aTm); #c1(COL6A5) c2(NP_001265227) c3(collagen alpha-5(VI) chain isoform 1 precursor) c4(97971, 97972, 97973, 97974, 97975) c5(97976, 97977, 97978, 97979, 97980, 97981, 97982, 97983, 97984, 97985, 97986, 97987, 97988, 97989, 97990, 97991, 97992, 97993, 97994, 97995, 97996, 97997, 97998, 97999) c6(98000, 98001, 98002, 98003, 98004, 98005) c7(Wn, aTi, arw, eE, bM); #c1(COL6A5) c2(NP_694996) c3(collagen alpha-5(VI) chain isoform 2 precursor) c4( ) c5(245620, 245621, 245622, 245623, 245624, 245625, 245626, 245627) c6(245628, 245629) c7(Wn, aTi, arw, eE, bM); #c1(COL6A5) c2(XP_011510923) c3(collagen alpha-5(VI) chain isoform X1) c4( ) c5(301398, 301399, 301400, 301401, 301402, 301403, 301404, 301405) c6(301406, 301407) c7(Wn, aTi, arw, eE, bM); #c1(COL7A1) c2(NP_000085) c3(collagen alpha-1(VII) chain precursor) c4(1416, 1417) c5(1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519) c6(1520, 1521, 1522, 1523, 1524) c7(T, yk, n, aTy, iD, ci, aTs, XM, aTr, alt, aT, rO, aTx, lQ, aIn, afu, aTp, aTv, aTq, aTo, ff, as, ark, en, dr, aTw, aAW, aTu, XN, adg, it); #c1(COL7A1) c2(XP_011531644) c3(collagen alpha-1(VII) chain isoform X7) c4( ) c5(327784, 327785) c6( ) c7(T, yk, n, aTy, iD, ci, als, XM, aTr, alt, aT, rO, aTx, lQ, aIn, afu, aTp, aTv, alq, aTo, ff, asl, ark, en, dr, aTw, aAW, aTu, XN, adg, it); #c1(COL8A1) c2(NP_065084) c3(collagen alpha-1(VIII) chain precursor) c4(203776, 203777) c5(203778, 203779, 203780, 203781, 203782, 203783, 203784, 203785, 203786, 203787, 203788, 203789, 203790, 203791, 203792, 203793) c6( ) c7(bL, Lu, alu, W, nb, pZ, lR, yL, EQ, Be); #c1(COL8A2) c2(NP_001281276) c3(collagen alpha-2(VIII) chain isoform 2) c4(111683) c5(111684, 111685, 111686, 111687, 111688, 111689, 111690, 111691, 111692, 111693, 111694, 111695, 111696, 111697, 111698, 111699, 11/700, 111701, 111702, 111703, 111704, 111705, 111706, 111707, 111708, 111709, 111710, 111711) c6( ) c7(Lu, alu, lR, pZ, aSP, ab, aou, K, aTz, B); #c1(COL9A1) c2(NP_001842) c3(collagen alpha-1(IX) chain isoform 1 precursor) c4(121644) c5(121645, 121646, 121647, 121648, 121649, 121650, 121651, 121652, 121653, 121654, 121655, 121656) c6( ) c7(eA, aR, aTB, XW, ec, ry, rl, h, By, rv, jf, lR, ih, dq, lv, aDQ, vk, BU, qe, mG, ln, Be, aDR, ER, aTA, R, zB); #c1(COL9A1) c2(NP_511040) c3(collagen alpha-1(IX) chain isoform 2 precursor) c4(231108, 231109) c5(231110, 231111, 231112, 231113, 231114, 231115, 231116, 231117, 231118, 231119, 231120, 231121) c6( ) c7(eA, aR, aTB, XW, ec, ry, rl, h, By, rv, jf, lR, ih, dq, lv, aDQ, vk, BU, qe, mG, ln, Be, aDR, ER, aTA, R, zB); #c1(COL9A1) c2(XP_011533731) c3(collagen alpha-1(IX) chain isoform X1) c4(329817) c5( ) c6( ) c7(eA, aR, aTB, XW, ec, ry, rl, h, By, rv, jf, lR, ih, dq, lv, aDQ, vK, BU, qe, mG, ln, Be, aDR, ER, aTA, R, zB); #c1 (COL9A2) c2(XP_011539020) c3(collagen alpha-2(IX) chain isoform X5) c4( ) c5(337084) c6( ) c7(aTD, aTE, aR, eA, ec, aTF, rl, ry, h, rv, jf, ih, dq, bQ, cM, lv, aik, vK, BU, qe, HP, ln, uo, aef, aTC, ER, R, zB, rp); #c1(COL9A3) c2(NP_001844) c3(collagen alpha-3(IX) chain precursor) c4(121657) c5(121658, 121659, 121660) c6(121661, 121662) c7(aj, bX, aTE, aR, J, eA, ec, aTF, rl, ry, h, bc, jf, A, Y, ih, dq, aTG, lv, aik, vk, BU, qe, ln, aef, ER, R, zB, rp); #c1(COL9A3) c2(XP_011526845) c3(collagen alpha-3(IX) chain isoform X1) c4(321198) c5( ) c6( ) c7(aj, bX, aTE, aR, J, eA, ec, aTF, rl, ry, h, bc, jf, A, Y, ih, dq, aTG, lv, aik, vK, BU, qe, ln, aef, ER, R, zB, rp); #c1(COL9A3) c2(XP_011526846) c3(collagen alpha-3(IX) chain isoform X2) c4( ) c5(321199, 321200, 321201, 321202, 321203, 321204, 321205, 321206, 321207, 321208) c6(321209, 321210) c7(aj, bX, aTE, aR, J, eA, ec, aTF, rl, ry, h, bc, jf, A, Y, ih, dq, aTG, lv, aik, vK, BU, qe, ln, aef, ER, R, zB, rp); #c1(COL9A3) c2(XP_011526849) c3(collagen alpha-3(IX) chain isoform X5) c4( ) c5(321211, 321212, 321213, 321214, 321215, 321216, 321217, 321218) c6(321219, 321220) c7(aj, bX, aTE, aR, J, eA, ec, aTF, rl, ry, h, bc, jf, A, Y, ih, dq, aTG, Iv, aik, vK, BU, qe, ln, aef, ER, R, zB, rp); #c1(COLCA1) c2(NP_001289574) c3(colorectal cancer-associated protein 1) c4(114281) c5(114282, 114283, 114284, 114285, 114286, 114287, 114288, 114289, 114290, 114291, 114292, 114293, 114294, 114295) c6( ) c7(bB, bE, q, r); #c1(COLCA2) c2(NP_001258386) c3(colorectal cancer-associated protein 2 isoform 2) c4( ) c5(96031, 96032, 96033, 96034, 96035, 96036, 96037, 96038, 96039, 96040, 96041, 96042, 96043) c6( ) c7(bB, bE, q, r); #c1(COLCA2) c2(NP_001258387) c3(colorectal cancer-associated protein 2 isoform 1) c4( ) c5(96044) c6( ) c7(bB, bE, q, r); #c1(COLCA2) c2(XP_011540891) c3(colorectal cancer-associated protein 2 isoform X1) c4(339434, 339435) c5(339436, 339437) c6( ) c7(bB, bE, q, r); #c1(COLEC10) c2(NP_006429) c3(collectin-10 precursor) c4(165141) c5(165142, 165143, 165144, 165145, 165146, 165147) c6( ) c7(0g, T, Of, jh, aj); #c1(COLEC11) c2(NP_001242912) c3(collectin-11 isoform d precursor) c4( ) c5(89241, 89242, 89243, 89244, 89245) c6( ) c7(asp, ac, aTl, Az, U, aTH, h, aTJ, dy); #c1(COLEC11) c2(NP_001242914) c3(collectin-11 isoform f precursor) c4(89246, 89247) c5(89248, 89249, 89250, 89251, 89252, 89253, 89254) c6( ) c7(asp, ac, aTl, Az, U, aTH, h, aTJ, dy); #c1(COLEC11) c2(NP_001242916) c3(collectin-11 isoform h) c4(89255) c5(89256, 89257, 89258, 89259, 89260, 89261) c6( ) c7(asp, ac, aTl, Az, U, aTH, h, aTJ, dy); #c1(COLEC11) c2(NP_001242917) c3(collectin-11 isoform i) c4(89262) c5(89263, 89264, 89265, 89266, 89267, 89268) c6( ) c7(asp, ac, aTl, Az, U, aTH, h, aTJ, dy); #c1(COLEC11) c2(NP_001242918) c3(collectin-11 isoform j) c4(89269) c5(89270, 89271, 89272, 89273, 89274, 89275) c6( ) c7(asp, ac, aTl, Az, U, aTH, h, aTJ, dy); #c1(COLEC11) c2(NP_076932) c3(collectin-11 isoform a precursor) c4( ) c5(214301, 214302, 214303, 214304, 214305) c6( ) c7(asp, ac, aTl, Az, U, aTH, h, aTJ, dy); #c1 (COLEC11) c2(NP_954705) c3(collectin-11 isoform b) c4(261326) c5(261327, 261328, 261329, 261330, 261331, 261332) c6( ) c7(asp, ac, aTl, Az, U, aTH, h, aTJ, dy); #c1(COLEC11) c2(XP_006711960) c3(collectin-11 isoform X2) c4(286308) c5(286309) c6( ) c7(asp, ac, aTl, Az, U, aTH, h, aTJ, dy); #c1(COLEC12) c2(NP_569057) c3(collectin-12) c4(233623) c5(233624, 233625, 233626, 233627, 233628, 233629) c6( ) c7(jW, Ae, oj, Ak, QK, lB); #c1(COLGALT2) c2(NP_001290349) c3(procollagen galactosyltransferase 2 isoform 2 precursor) c4(115221) c5(115222, 115223, 115224, 115225, 115226, 115227) c6( ) c7(an); #c1(COLGALT2) c2(NP_001290350) c3(procollagen galactosyltransferase 2 isoform 3) c4( ) c5(115228, 115229, 115230, 115231, 115232, 115233) c6( ) c7(an); #c1(COLGALT2) c2(NP_055916) c3(procollagen galactosyltransferase 2 isoform 1precursor) c4(184466, 184467) c5(184468, 184469, 184470, 184471, 184472, 184473) c6( ) c7(an); #c1(COLGALT2) c2(XP_011507640) c3(procollagen galactosyltransferase 2 isoform X4) c4(297580) c5(297581, 297582, 297583, 297584, 297585, 297586) c6( ) c7(an); #c1(COLQ) c2(NP_005668) c3(acetylcholinesterase collagenic tail peptide isoform 1 precursor) c4(158385, 158386, 158387) c5(158388, 158389, 158390, 158391, 158392, 158393, 158394, 158395, 158396, 158397, 158398) c6( ) c7(xl, xA, wX); #c1(COLQ) c2(NP_536799) c3(acetylcholinesterase collagenic tail peptide isoform 11 precursor) c4(231332, 231333) c5(231334) c6( ) c7(xl, xA, wX); #c1(COLQ) c2(NP_536800) c3(acetylcholinesterase collagenic tail peptide isoform 111 precursor) c4(231335, 231336) c5(231337, 231338, 231339, 231340, 231341, 231342, 231343, 231344, 231345, 231346, 231347, 231348, 231349, 231350, 231351, 231352, 231353, 231354, 231355, 231356, 231357, 231358, 231359, 231360, 231361, 231362, 231363, 231364, 231365, 231366, 231367, 231368, 231369, 231370, 231371, 231372) c6( ) c7(xl, xA, wX); #c1(COMMD1) c2(NP_689729) c3(COMM domain-containing protein 1) c4(243701, 243702, 243703, 243704, 243705) c5(243706, 243707, 243708, 243709, 243710, 243711, 243712, 243713, 243714, 243715, 243716, 243717, 243718, 243719, 243720, 243721, 243722, 243723, 243724, 243725, 243726, 243727, 243728, 243729, 243730, 243731, 243732, 243733, 243734, 243735, 243736, 243737, 243738, 243739, 243740, 243741, 243742, 243743, 243744, 243745, 243746, 243747, 243748, 243749, 243750, 243751, 243752, 243753, 243754, 243755, 243756, 243757, 243758, 243759, 243760, 243761, 243762, 243763, 243764, 243765, 243766, 243767, 243768, 243769, 243770, 243771, 243772, 243773, 243774, 243775, 243776, 243777, 243778, 243779, 243780, 243781, 243782, 243783, 243784, 243785, 243786, 243787, 243788, 243789, 243790, 243791, 243792, 243793, 243794, 243795, 243796, 243797, 243798, 243799, 243800) c6(243801) c7(eY, T, aaQ, aTK, al, ep, aTL, h, dq, fe, ji, iV, als, ao, vi); #c1(COMMD1) c2(XP_011530860) c3(COMM domain-containing protein 1 isoform X1) c4( ) c5(326856, 326857, 326858, 326859, 326860, 326861, 326862) c6( ) c7(eY, T, aaQ, aTK, al, ep, aTL, h, dq, fe, ji, iV, als, ao, vi); #c1(COMMD1) c2(XP_011530861) c3(COMM domain-containing protein 1 isoform X2) c4(326863, 326864, 326865) c5( ) c6( ) c7(eY, T, aaQ, aTK, al, ep, aTL, h, dq, fe, ji, iV, als, ao, vi); #c1(COMMD1) c2(XP_011530862) c3(COMM domain-containing protein 1 isoform X3) c4( ) c5(326866, 326867, 326868, 326869, 326870, 326871, 326872, 326873, 326874, 326875, 326876, 326877, 326878, 326879, 326880) c6( ) c7(eY, T, aaQ, aTK, al, ep, aTL, h, dq, fe, ji, iV, als, ao, vi); #c1(COMMD10) c2(NP_057228) c3(COMM domain-containing protein 10) c4(189599) c5(189600, 189601, 189602, 189603, 189604, 189605) c6( ) c7(c); #c1(COMMD3-BMI1) c2(NP_001190991) c3(COMMD3-BMI1 read-through protein) c4(80414, 80415, 80416, 80417, 80418, 80419, 80420, 80421, 80422) c5(80423, 80424, 80425, 80426, 80427, 80428, 80429, 80430, 80431, 80432, 80433, 80434, 80435, 80436, 80437, 80438, 80439, 80440) c6( ) c7(T, if, fd, bB, ci, x, aE, SV, ja, K, hu, DY, pk, eL, yo, aW, ark, as, eF, cm, bw, eM, m, k, ht, n, qN, br, Bl, ft, jz, eV, hn, dL, asH, dx, pl, oS, A, aq, kp, oU, Qy, bP, asl, dB, hk, Qu, hR, nz, it, B, bL, ul, fM, dA, yk, eX, aA, q, eA, asG, MR, aoA, aT, cy, ae, jf, Uf, iU, oV, Y, r, en, cl, t, ik, ck, QG, gX, rA, bt, KG, Mb, jh, Gn, hF, ad, d, qt, ff, eH, cr, Ej, aF, iR, bz, bE, bC, iu); #c1(COMMD5) c2(NP_054785) c3(COMM domain-containing protein 5) c4(177818) c5(177819, 177820, 177821, 177822, 177823, 177824, 177825, 177826, 177827, 177828, 177829, 177830, 177831, 177832) c6( ) c7(bL, cy, cz, dy, aA); #c1(COMMD7) c2(NP_001092809) c3(COMM domain-containing protein 7 isoform 2) c4(39654) c5(39655, 39656, 39657, 39658, 39659, 39660) c6( ) c7(aw, B); #c1(COMMD7) c2(NP_444269) c3(COMM domain-containing protein 7 isoform 1) c4(230215) c5(230216, 230217, 230218, 230219, 230220, 230221) c6( ) c7(aw, B); #c1(COMP) c2(NP_000086) c3(cartilage oligomeric matrix protein precursor) c4(1525, 1526, 1527) c5(1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551) c6( ) c7(dD, ea, aTN, bB, j, ade, eq, lR, ai, aR, Pm, aRo, eA, aHF, xE, ec, rw, ry, h, pj, rv, af, jf, DI, lU, ih, dq, pl, aOm, pb, lv, LB, vK, sS, BU, qe, bE, ln, aoJ, ER, R, zB, aTM, y); #c1 (COMT) c2(NP_001128634) c3(catechol O-methyltransferase isoform MB-COMT) c4(50391, 50392) c5(50393, 50394, 50395, 50396, 50397, 50398, 50399, 50400, 50401, 50402, 50403, 50404, 50405, 50406, 50407, 50408, 50409, 50410, 50411) c6( ) c7(aUh, T, aUk, ea, aqo, abM, e, bO, eb, bB, yY, gf, Jd, ci, aTS, ur, HH, Jt, qj, Gp, aQF, gF, aR, lS, VV, aEd, acc, iX, M, ta, K, oi, eL, xa, gr, zQ, aW, hl, g, sz, Eg, uC, mZ, aaS, fT, yl, eM, nB, lp, iA, wE, vA, ZT, Gs, U, Ji, sv, cX, aTV, m, abl, uJ, xh, aTW, aCS, yA, gL, nD, FW, dD, RX, ht, NT, aw, n, To, aj, fr, ct, nA, ml, ww, aUf, agJ, eW, hw, axc, aNU, aUb, dL, aUm, Gw, mV, mY, aTX, aTO, alY, aUi, he, mJ, mU, abf, PT, rk, Fn, ny, sp, aq, vo, cM, Ld, op, cv, mX, hq, aUn, ok, abV, cA, anZ, Pl, yP, Ei, hL, at, ut, gv, lB, it, B, mT, jd, qc, bL, jc, jv, iH, eR, He, jx, aOH, aTP, aA, mS, ti, cz, j, hy, v, aql, wy, V, q, aqN, PW, gM, cF, acj, HJ, aTT, aTZ, aql, aT, wP, bK, bq, an, rz, rE, ae, af, bA, qw, kl, yL, zS, abi, r, cu, eS, HL, en, cl, t, aU, xw, tl, bN, aUc, pH, azK, mf, aTU, FI, GI, qX, VR, ob, akd, aZ, aD, iW, ha, jh, bS, uu, eq, qi, agY, ad, aUld, aUe, dP, HF, agd, F, ot, ib, mW, sO, aqk, Sb, xp, aUj, na, hZ, WZ, aU, aog, tb, BO, PE, Gv, ff, gC, gl, aTY, aF, aTR, gu, Z, FX, iR, bz, jb, Ek, es, D, gk, aHg, bE, HP, pP, aHa, Rm, ei, ra, pk, Gu, rF, xg, X, agZ, pC); #c1(COMT) c2(NP_009294) c3(catechol 0-methyltransferase isoform S-COMT) c4(171797) c5(171798, 171799, 171800, 171801, 171802, 171803, 171804, 171805) c6( ) c7(aUh, T, aUk, ea, aqo, abM, e, bO, eb, bB, yY, gf, Jd, ci, aTS, ur, HH, Jt, qj, Gp, aQF, gF, aR, lS, VV, aEQ, acc, ix, M, ta, K, oi, eL, xa, gr, zQ, aW, hl, g, sz, Eg, uC, mZ, aaS, fT, yl, eM, nB, lp, iA, WE, vA, ZT, Gs, U, Ji, sv, cX, aTV, m, abl, uJ, xh, aTW, aCS, yA, gL, nD, FW, dD, RX, ht, NT, aw, n, To, aj, fr, ct, nA, ml, ww, aUf, agJ, eW, hw, axc, aNU, aUb, dL, aUm, Gw, mV, mY, aTX, aTO, alY, aUi, he, mJ, mU, abf, PT, rk, Fn, ny, sp, aq, vo, cM, Ld, op, cv, mX, hq, aUn, ok, abV, cA, anZ, Pl, yP, Ei, hL, at, ut, gv, IB, it, B, mT, jd, qc, bL, jc, jv, iH, eR, He, jx, aOH, aTP, aA, mS, ti, cz, j, hy, v, aql, wy, V, q, aqN, PW, gM, cF, acj, HJ, aTT, aTZ, aql, aT, wP, bK, bq, an, rz, rE, ae, af, bA, qw, kl, yL, zS, abi, r, cu, eS, HL, en, cl, t, aTQ, xw, tl, bN, aUc, pH, azK, mf, aTU, FI, GI, qX, VR, ob, akd, aZ, aD, iW, ha, jh, bS, uu, eq, qi, agY, ad, aUld, ale, dP, HF, agd, F, ot, ib, mW, sO, aqk, Sb, xp, aUj, na, hZ, WZ, aU, aog, tb, BO, PE, Gv, ff, gC, gl, aTY, aF, aTR, gu, Z, FX, iR, bz, jb, Ek, es, D, gk, aHg, bE, HP, pP, aHa, Rm, ei, ra, pk, Gu, rF, xg, X, agZ, pC); #c1(COMT) c2(XP_011528188) c3(catechol O-methyltransferase isoform X2) c4( ) c5(323287, 323288, 323289) c6( ) c7(aUh, T, aUk, ea, aqo, abM, e, bO, eb, bB, yY, gf, Jd, ci, aTS, ur, HH, Jt, qj, Gp, aQF, gF, aR, lS, W, aEQ, acc, iX, M, ta, K, oi, eL, xa, gr, zQ, aW, hl, g, sz, Eg, uC, mZ, aaS, fT, yl, eM, nB, lp, iA, wE, vA, ZT, Gs, U, Ji, sv, cX, aTV, m, abl, uJ, xh, aTW, aCS, yA, gL, nD, FW, dD, RX, ht, NT, aw, n, To, aj, fr, ct, nA, ml, ww, aUf, agJ, eW, hw, axc, aNU, aUb, dL, aUm, Gw, mV, mY, aTX, aTO, alY, ali, he, mJ, mu, abf, PT, rk, Fn, ny, sp, aq, vo, cM, Ld, op, cv, mX, hq, aUn, ok, abV, cA, anZ, Pl, yP, Ei, hL, at, ut, gv, lB, it, B, mT, jd, qc, bL, jc, jv, iH, eR, He, jx, aOH, aTP, aA, mS, ti, cz, j, hy, v, aql, wy, V, q, aqN, PW, gM, cF, acj, HJ, aTT, aTZ, aql, aT, wP, bK, bq, an, rz, rE, ae, af, bA, qw, kl, yL, zS, abi, r, cu, eS, HL, en, cl, t, aU, xw, tl, bN, aUc, pH, azk, mf, aTU, FI, GI, qX, VR, ob, akd, aZ, aD, iW, ha, jh, bS, uu, eq, qi, agY, ad, aUd, ale, dP, HF, agd, F, ot, ib, mW, sO, aqk, Sb, xp, alj, na, hZ, WZ, aU, aog, tb, BO, PE, Gv, ff, gC, gl, aTY, aF, aTR, gu, Z, FX, iR, bz, jb, Ek, es, D, gk, aHg, bE, HP, pP, aHa, Rm, ei, ra, pk, Gu, rf, xg, X, agZ, pC); #c1 (COPA) c2(NP_001091868) c3(coatomer subunit alpha isoform 1) c4(38734) c5(38735, 38736, 38737, 38738, 38739, 38740) c6( ) c7(bL, T, ib, kf, dG, ae, B, ad); #c1(COPA) c2(NP_004362) c3(coatomer subunit alpha isoform 2) c4(145712) c5(145713, 145714, 145715, 145716, 145717, 145718) c6( ) c7(bL, T, ib, kf, dG, ae, B, ad); #c1(COPB1) c2(NP_057535) c3(coatomer subunit beta) c4(191282) c5(191283, 191284, 191285, 191286, 191287, 191288, 191289, 191290, 191291, 191292) c6( ) c7(y); #c1(COPB2) c2(NP_004757) c3(coatomer subunit beta′) c4(149351, 149352) c5(149353, 149354, 149355, 149356, 149357, 149358, 149359, 149360, 149361, 149362, 149363, 149364) c6( ) c7(NK, d, h, K, lf, y); #c1(COPE) c2(NP_009194) c3(coatomer subunit epsilon isoform a) c4(171423, (71424) c5(171425, 171426, 171427, 171428, 171429, 171430, 171431, 171432, 171433, 171434, 171435, 171436) c6( ) c7(ad); #c1(COPE) c2(NP_955474) c3(coatomer subunit epsilon isoform b) c4(261835) c5(261836, 261837, 261838, 261839, 261840, 261841) c6( ) c7(ad); #c1(COPE) c2(NP_955476) c3(coatomer subunit epsilon isoform c) c4(261849) c5(261850, 261851, 261852, 261853, 261854, 261855) c6( ) c7(ad); #c1(COPG2) c2(NP_001276962) c3(coatomer subunit gamma-2 isoform 2) c4(108844) c5(108845, 108846, 108847, 108848, 108849, 108850, 108851, 108852, 108853, 108854) c6( ) c7(LO); #c1(COPG2) c2(NP_036265) c3(coatomer subunit gamma-2 isoform 1) c4(172699) c5(172700, 172701, 172702, 172703, 172704, 172705) c6( ) c7(LO); #c1(COPRS) c2(NP_060875) c3(coordinator of PRMTS and differentiation stimulator) c4(198230) c5(198231, 198232, 198233, 198234, 198235, 198236) c6( ) c7(hP, rY); #c1(COPRS) c2(XP_005258057) c3(coordinator of PRMTS and differentiation stimulator isoform X1) c4(275472) c5(275473, 275474, 275475, 275476, 275477, 275478, 275479) c6( ) c7(hP, rY); #c1(COPS2) c2(NP_001137359) c3(COP9 signalosome complex subunit 2 isoform 2) c4(55606) c5(55607, 55608, 55609, 55610, 55611, 55612) c6( ) c7(dP, aU); #c1(COPS2) c2(NP_004227) c3(COP9 signalosome complex subunit 2 isoform 1) c4(144457) c5(144458, 144459, 144460, 144461, 144462, 144463, 144464) c6( ) c7(dP, aU); #c1(COPS3) c2(NP_001186054) c3(COP9 signalosome complex subunit 3 isoform 2) c4( ) c5(77000, 77001, 77002, 77003, 77004) c6( ) c7(gk, al, eH, eF, V, B); #c1(COPS3) c2(NP_003644) c3(COP9 signalosome complex subunit 3 isoform 1) c4(139871, 139872, 139873, 139874) c5(139875, 139876, 139877, 139878, 139879, 139880, 139881, 139882, 139883, 139884, 139885, 139886, 139887) c6( ) c7(gk, al, eH, eF, V, B); #c1(COPS4) c2(NP_001244935) c3(COP9 signalosome complex subunit 4 isoform 2) c4(92335) c5(92336, 92337, 92338, 92339, 92340, 92341) c6( ) c7(cm, f); #c1(COPS4) c2(NP_057213) c3(COP9 signalosome complex subunit 4 isoform 1) c4(189523) c5(189524, 189525, 189526, 189527, 189528, 189529) c6( ) c7(cm, f); #c1(COPS5) c2(NP_006828) c3(COP9 signalosome complex subunit 5) c4(168489) c5(168490, 168491, 168492, 168493, 168494, 168495, 168496) c6( ) c7(T, aC, al, jh, n, ci, q, hX, aE, db, aT, on, pl, iU, aW, Y, ff, r, en, t, nC, es, gk, bP, iA, m, B, vi); #c1(COPS6) c2(NP_006824) c3(COP9 signalosome complex subunit 6) c4(168439, 168440) c5(168441, 168442, 168443, 168444, 168445, 168446, 168447, 168448, 168449, 168450, 168451, 168452) c6( ) c7(h, aW, nB, aT); #c1(COPS7A) c2(XP_005253752) c3(COP9 signalosome complex subunit 7a isoform X1) c4(272641) c5(272642, 272643, 272644, 272645, 272646, 272647, 272648, 272649, 272650, 272651, 272652, 272653, 272654, 272655, 272656, 272657, 272658, 272659, 272660, 272661, 272662, 272663, 272664, 272665, 272666, 272667) c6( ) c7(aA); #c1(COPS8) c2(NP_006701) c3(COP9 signalosome complex subunit 8 isoform 1) c4(167245, 167246) c5(167247, 167248, 167249, 167250, 167251, 167252, 167253) c6( ) c7(aW, T, nB, aT); #c1(COPS8) c2(NP_937832) c3(COP9 signalosome complex subunit 8 isoform 2) c4( ) c5(259253, 259254, 259255, 259256, 259257) c6( ) c7(aW, T, nB, aT); #c1(COPZ2) c2(NP_057513) c3(coatomer subunit zeta-2) c4(191200) c5(191201, 191202, 191203, 191204, 191205, 191206, 191207, 191208) c6( ) c7(hL, c1); #c1(C002) c2(NP_056512) c3(4-hydroxybenzoate polyprenyltransferase, mitochondrial) c4(187851, 187852, 187853) c5(187854, 187855, 187856, 187857, 187858, 187859, 187860, 187861, 187862, 187863, 187864, 187865, 187866, 187867, 187868, 187869, 187870, 187871) c6( ) c7(EG, eR, ky, kB, mG, Ju, oj, dV, aUo, EF, GC, dy, ah, eh, ajU, dR); #c1(COQ2) c2(XP_011530157) c3(4-hydroxybenzoate polyprenyltransferase, mitochondrial isoform X1) c4( ) c5(326050) c6( ) c7(EG, eR, ky, kB, mG, Ju, oj, dV, aUo, EF, GC, dy, ah, eh, aju, dR); #c1(C003) c2(NP_059117) c3(hexaprenyldihydroxybenzoate methyltransferase, mitochondrial) c4(193128) c5(193129, 193130, 193131, 193132, 193133, 193134) c6( ) c7(lp); #c1(C003) c2(XP_005267075) c3(hexaprenyldihydroxybenzoate methyltransferase, mitochondrial isoform X3) c4(280669) c5(280670, 280671, 280672, 280673, 280674, 280675, 280676, 280677, 280678, 280679) c6( ) c7(lp); #c1(C003) c2(XP_011534200) c3(hexaprenyldihydroxybenzoate methyltransferase, mitochondrial isoform X2) c4( ) c5(330503, 330504, 330505) c6( ) c7(lp); #c1(C044) c2(NP_057119) c3(ubiquinone biosynthesis protein COQ4 homolog, mitochondrial precursor) c4(188987, 188988) c5(188989, 188990, 188991, 188992, 188993, 188994) c6( ) c7(EG); #c1(COQ4) c2(XP_011517063) c3(ubiquinone biosynthesis protein COQ4 homolog, mitochondrial isoform X1) c4( ) c5(310556, 310557, 310558, 310559, 310560, 310561) c6( ) c7(EG); #c1(C005) c2(NP_115690) c3(2-methoxy-6-polyprenyl-1,4-benzoquinol methylase, mitochondrial precursor) c4(223564, 223565) c5(223566, 223567, 223568, 223569, 223570, 223571, 223572, 223573, 223574, 223575, 223576, 223577) c6( ) c7(nA); #c1(C006) c2(NP_872282) c3(ubiquinone biosynthesis monooxygenase COQ6 isoform a) c4(255996, 255997) c5(255998, 255999, 256000, 256001, 256002, 256003) c6( ) c7(ah, mG, gi, aUp); #c1(C006) c2(NP_872286) c3(ubiquinone biosynthesis monooxygenase COQ6 isoform b) c4(256004) c5(256005, 256006, 256007, 256008, 256009, 256010) c6( ) c7(ah, mG, gi, aUp); #c1(COQ6) c2(XP_005267773) c3 (ubiquinone biosynthesis monooxygenase COQ6 isoform X2) c4(281236) c5(281237, 281238, 281239, 281240, 281241, 281242, 281243, 281244, 281245) c6( ) c7(ah, mG, gi, aUp); #c1(COQ6) c2(XP_011535111) c3(ubiquinone biosynthesis monooxygenase COQ6 isoform X3) c4(331926, 331927, 331928) c5(331929, 331930, 331931, 331932, 331933, 331934, 331935, 331936, 331937, 331938, 331939, 331940, 331941, 331942, 331943) c6( ) c7(ah, mG, gi, aUp); #c1(C006) c2(XP_011535112) c3(ubiquinone biosynthesis monooxygenase COQ6 isoform X5) c4( ) c5(331944, 331945, 331946, 331947, 331948, 331949) c6( ) c7(ah, mG, gi, aUp); #c1(C007) c2(NP_001177912) c3(ubiquinone biosynthesis protein COQ7 homolog isoform 2) c4( ) c5(73134, 73135, 73136, 73137, 73138) c6( ) c7(asp, gi, U); #c1(COQ7) c2(NP_057222) c3(ubiquinone biosynthesis protein COQ7 homolog isoform 1) c4(189554, 189555) c5(189556, 189557, 189558, 189559, 189560, 189561, 189562) c6( ) c7(asp, gi, U); #c1(C009) c2(NP_064708) c3(ubiquinone biosynthesis protein COQ9, mitochondrial precursor) c4(203514) c5(203515, 203516, 203517, 203518, 203519, 203520) c6( ) c7(EG, EF, aUq, kC, aUr); #c1(CORIN) c2(NP_006578) c3(atrial natriuretic peptide-converting enzyme isoform 1) c4(166356, 166357, 166358) c5(166359, 166360, 166361, 166362, 166363, 166364, 166365, 166366, 166367, 166368, 166369, 166370, 166371, 166372, 166373, 166374, 166375, 166376) c6( ) c7(gx, jv, oe, aDT, cz, eb, j, zU, cd, ft, zw, aUu, h, aUs, bA, hM, iV, aUv, xA, xj, cj, aUt); #c1(CORDIA) c2(XP_011544016) c3(coronin-IA isoform X1) c4(343180) c5(343181, 343182, 343183, 343184, 343185, 343186, 343187, 343188, 343189, 343190, 343191) c6( ) c7(T, ul, dA, jh, aA, aGx, ci, mC, LH, x, aT, iL, zQ, ZH, CN, aW, ff, eZ, uY, aF, en, t, ck, aUw, k, m, fl, bt, B); #c1(CORO1B) c2(NP_001018080) c3(coronin-(B) c4(27107, 27108, 27109, 27110) c5(27111, 27112, 27113, 27114, 27115, 27116, 27117, 27118, 27119, 27120, 27121, 27122, 27123, 27124, 27125, 27126, 27127, 27128, 27129, 27130, 27131, 27132, 27133, 27134, 27135, 27136, 27137, 27138) c6( ) c7(By); #c1 (CORO1C) c2(NP_001098707) c3(coronin-IC isoform a) c4(41491, 41492, 41493) c5(41494, 41495, 41496, 41497, 41498, 41499, 41500, 41501, 41502, 41503, 41504, 41505, 41506, 41507, 41508, 41509, 41510, 41511, 41512, 41513, 41514, 41515, 41516) c6( ) c7(bL, T, d, eM, dB, aA, af, ey, eA, B); #c1 (CORD1C) c2(NP_001263400) c3(coronin-IC isoform b) c4(97258, 97259, 97260, 97261, 97262, 97263) c5( ) c6( ) c7(bL, T, d, eM, dB, aA, aF, ey, eA, B); #c1(CORO1C) c2(XP_011536426) c3(coronin-IC isoform X1) c4( ) c5(333779, 333780, 333781, 333782, 333783, 333784, 333785, 333786, 333787, 333788, 333789, 333790, 333791, 333792, 333793, 333794, 333795, 333796, 333797, 333798, 333799, 333800, 333801, 333802, 333803, 333804, 333805, 333806, 333807) c6( ) c7(bL, T, d, eM, dB, aA, aF, ey, eA, B); #c1(CORO1C) c2(XP_011536427) c3(coronin-IC isoform X2) c4( ) c5(333808, 333809, 333810, 333811, 333812, 333813) c6( ) c7(bL, T, d, eM, dB, aA, aF, ey, eA, B); #c1(CORO2A) c2(XP_011517288) c3(coronin-2A isoform X1) c4(310882) c5(310883, 310884, 310885, 310886, 310887, 310888, 310889, 310890, 310891, 310892) c6( ) c7(q, qj, r); #c1(CORO2B) c2(NP_001177385) c3(coronin-2B isoform 2) c4( ) c5(72734, 72735, 72736, 72737, 72738, 72739, 72740, 72741, 72742) c6( ) c7(ht, EQ); #c1(CORO2B) c2(NP_006082) c3(coronin-28 isoform 1) c4(161759, 161760) c5(161761, 16/762, 16/763, 161764, 161765, 16/766, 161767, 16/768, 161769, 161770) c6( ) c7(ht, EQ); #c1(CORO2B) c2(XP_005254181) c3(coronin-28 isoform X2) c4( ) c5(273050) c6( ) c7(ht, EQ); #c1(CORO6) c2(NP_116243) c3(coronin-6) c4(225919, 225920) c5(225921, 225922, 225923, 225924, 225925, 225926, 225927, 225928, 225929, 225930, 225931, 225932) c6( ) c7(fr); #c1(CORO6) c2(XP_005258113) c3(coronin-6 isoform X9) c4(275502) c5(275503, 275504, 275505, 275506, 275507, 275508) c6( ) c7(fr); #c1(CORO6) c2(XP_011523687) c3(coronin-6 isoform X3) c4(318166) c5(318167, 318168, 318169, 318170, 318171, 318172) c6( ) c7(fr); #c1(CORO7) c2(NP_001188402) c3(coronin-7 isoform 3) c4(79120) c5(79121, 79122, 79123, 79124, 79125, 79126, 79127, 79128, 79129, 79130, 79131, 79132, 79133, 79134, 79135, 79136, 79137, 79138, 79139, 79140, 79141) c6( ) c7(iv, iw); #c1(CORO7) c2(NP_078811) c3(coronin-7 isoform 1) c4( ) c5(215588, 215589, 215590, 215591, 215592, 215593) c6( ) c7(iv, iw); #c1(CORO7-PAM16) c2(NP_001188408) c3(CORO7-PAM16 protein) c4(79142, 79143, 79144, 79145) c5(79146, 79147, 79148, 79149, 79150, 79151, 79152) c6( ) c7(iv, iw); #c1(COTL1) c2(NP_066972) c3(coactosin-like protein) c4(207915) c5(207916, 207917, 207918, 207919, 207920, 207921) c6( ) c7(Na, azs, ft, eM, amJ, eH, eZ, aT, Nc, aml, eF, y); #c1(COX10) c2(XP_005256515) c3(protoheme IX farnesyltransferase, mitochondrial isoform X3) c4( ) c5(274189, 274190, 274191, 274192, 274193, 274194) c6( ) c7(bL, oW, vc, aUx, aNG, acJ, cb, afO, af, ajU); #c1(COX10) c2(XP_011521960) c3(protoheme IX farnesyltransferase, mitochondrial isoform X2) c4(316678) c5(316679, 316680, 316681, 316682, 316683, 316684, 316685, 316686, 316687, 316688) c6( ) c7(bL, oW, vc, aUx, aNG, acJ, cb, afO, af, ajU); #c1(COX11) c2(NP_001156333) c3(cytochrome c oxidase assembly protein COX11, mitochondrial isoform 2) c4( ) c5(61704) c6( ) c7(aT); #c1(COX11) c2(NP_001156334) c3(cytochrome c oxidase assembly protein COX11, mitochondrial isoform 3) c4(61705) c5(61706, 61707, 61708, 61709, 61710, 61711) c6( ) c7(aT); #c1(COX11) c2(XP_011522644) c3(cytochrome c oxidase assembly protein COX11, mitochondrial isoform X1) c4(317197) c5(317198, 317199, 317200, 317201, 317202, 317203) c6( ) c7(aT); #c1(COX(4) c2(NP_001244062) c3(cytochrome c oxidase assembly protein COX14) c4(91860) c5(91861, 91862, 91863, 91864, 91865, 91866, 91867, 91868, 91869, 91870, 91871, 91872, 91873, 91874, 91875, 91876, 91877, 91878) c6( ) c7(acJ); #c1(COX15) c2(NP_004367) c3(cytochrome c oxidase assembly protein COX15 homolog isoform 2) c4(145776) c5(145777, 145778, 145779, 145780, 145781, 145782) c6( ) c7(cc, vc, aUx, acJ, ally, af, ajU); #c1(COX15) c2(NP_510870) c3(cytochrome c oxidase assembly protein COX15 homolog isoform 1) c4(231067) c5(231068, 231069, 231070, 231071, 231072, 231073, 231074, 231075) c6( ) c7(cc, vc, aUx, acJ, aUy, af, ajU); #c1(COX16) c2(NP_057552) c3(cytochrome c oxidase assembly protein COX16 homolog, mitochondrial isoform 1 precursor) c4(191356) c5(191357, 191358, 191359, 191360, 191361, 191362) c6( ) c7(acJ); #c1(COX17) c2(NP_005685) c3(cytochrome c oxidase copper chaperone) c4(158550, 158551) c5(158552, 158553, 158554, 158555, 158556, 158557, 158558, 158559, 158560, 158561, 158562, 158563, 158564, 158565, 158566, 158567, 158568, 158569, 158570, 158571) c6( ) c7(T, eM); #c1(COX18) c2(NP_001284661) c3(mitochondrial inner membrane protein COX18 isoform 1) c4(112274) c5(112275, 112276, 112277, 112278, 112279, 112280) c6( ) c7(hy, ne, BN); #c1(COX18) c2(NP_776188) c3(mitochondrial inner membrane protein COX18 isoform 2) c4(250957) c5(250958, 250959, 250960, 250961, 250962, 250963) c6( ) c7(hy, ne, BN); #c1(COX19) c2(NP_001026788) c3(cytochrome c oxidase assembly protein COX19) c4(29626) c5(29627, 29628, 29629, 29630, 29631, 29632) c6( ) c7(acJ); #c1(COX411) c2(NP_001852) c3(cytochrome c oxidase subunit 4 isoform 1, mitochondrial precursor) c4( ) c5(121684, 121685, 121686, 121687, 121688, 121689) c6( ) c7(acJ, v, awq, oj, h); #c1(COX411) c2(XP_011521173) c3(cytochrome c oxidase subunit 4 isoform 1, mitochondrial isoform X2) c4(316115) c5( ) c6( ) c7(acJ, v, awq, oj, h); #c1(COX412) c2(NP_115998) c3(cytochrome c oxidase subunit 4 isoform 2, mitochondrial) c4(225183) c5(225184, 225185, 225186, 225187, 225188, 225189) c6( ) c7(oW, oj, h, v, acJ, aUz, aKG, ad); #c1(COX5A) c2(NP_004246) c3(cytochrome c oxidase subunit 5A, mitochondrial precursor) c4(144574) c5(144575, 144576, 144577, 144578, 144579, 144580) c6( ) c7(cw, bM, T, aC, BD, aDy, sw, bB, n, aNG, j, q, eA, iu, aE, aT, cy, bl, K, aUA, af, ajU, Nr, s, aW, ff, eH, aNq, r, Ej, po, kp, aB, en, eF, t, kC, bz, Nq, sS, eM, SE, xZ, wD, bE, at, acJ, m, y, it, B); #c1(COX5B) c2(NP_001853) c3(cytochrome c oxidase subunit 58, mitochondrial precursor) c4(121690) c5(121691, 121692, 121693, 121694, 121695, 121696) c6( ) c7(bL, ac, cz, j, ci, anW); #c1(COX6A1) c2(NP_004364) c3(cytochrome c oxidase subunit GA1, mitochondrial) c4(145719, 145720) c5(145721, 145722, 145723, 145724, 145725, 145726, 145727, 145728, 145729, 145730, 145731, 145732) c6( ) c7(acJ, bU); #c1(COX6A2) c2(NP_005196) c3(cytochrome c oxidase subunit 6A2, mitochondrial precursor) c4(153812) c5(153813, 153814, 153815, 153816, 153817, 153818) c6( ) c7(acJ); #c1(COX6B1) c2(NP_001854) c3(cytochrome c oxidase subunit 681) c4(121697, 121698) c5(121699, 121700, 121701, 121702, 121703, 12/704, 121705, 121706, 121707, 121708, 121709, 121710) c6( ) c7(acJ); #c1(COX6C) c2(NP_004365) c3(cytochrome c oxidase subunit 6C proprotein) c4(145733) c5(145734, 145735, 145736, 145737, 145738, 145739, 145740, 145741, 145742, 145743, 145744, 145745, 145746, 145747, 145748, 145749, 145750, 145751, 145752, 145753, 145754, 145755, 145756, 145757, 145758, 145759, 145760, 145761, 145762, 145763, 145764, 145765, 145766, 145767, 145768, 145769, 145770, 145771, 145772, 145773, 145774, 145775) c6( ) c7(vA, ad); #c1(COX7A1) c2(NP_001855) c3(cytochrome c oxidase subunit 7A1, mitochondrial precursor) c4(121711, 121712) c5(121713, 121714, 121715, 121716, 121717, 121718, 121719, 121720, 121721, 121722, 121723, 121724) c6( ) c7(bL, iK, acJ, wm, wu); #c1(COX7A2) c2(XP_011533748) c3(cytochrome c oxidase subunit 7A2, mitochondrial isoform X1) c4(329854, 329855) c5(329856, 329857, 329858, 329859, 329860, 329861, 329862, 329863, 329864, 329865, 329866, 329867, 329868, 329869, 329870, 329871) c6( ) c7(ad); #c1(COX7A2L) c2(XP_011531460) c3(cytochrome c oxidase subunit 7A-related protein, mitochondrial isoform X1) c4(327585) c5(327586, 327587, 327588, 327589, 327590, 327591, 327592, 327593, 327594, 327595) c6( ) c7(bF, ou, ad, r); #c1(COX7B) c2(NP_001857) c3(cytochrome c oxidase subunit 78, mitochondrial precursor) c4(12/725) c5(121726, 121727, 121728, 121729, 121730, 121731) c6( ) c7(kC, aUB, kj); #c1(COX7B2) c2(XP_005248113) c3(cytochrome c oxidase subunit 782, mitochondrial isoform X1) c4(268212, 268213, 268214) c5(268215, 268216, 268217, 268218, 268219, 268220, 268221, 268222, 268223, 268224, 268225, 268226, 268227, 268228, 268229, 268230, 268231, 268232, 268233, 268234, 268235, 268236, 268237, 268238, 268239, 268240) c6( ) c7(ci, ad); #c1(COX7C) c2(NP_001858) c3(cytochrome c oxidase subunit 7C, mitochondrial precursor) c4(121732) c5(121733, 121734, 121735, 121736, 121737, 121738, 121739, 121740, 121741, 121742) c6( ) c7(aw, v, cd); #c1(COX8A) c2(NP_004065) c3(cytochrome c oxidase subunit 8A, mitochondrial) c4(143252, 143253) c5(143254, 143255, 143256, 143257, 143258, 143259, 143260, 143261) c6( ) c7(bM, T, aC, aSz, al, fd, bB, aiC, ame, ip, x, aE, rw, Qk, M, dV, adB, K, dF, aW, hl, axJ, aNq, YG, aB, aUG, Ku, g, as, kJ, eF, DL, aiv, bw, aUF, ba, wE, uo, wx, k, m, Ro, P, NU, NK, aUL, aUK, Zo, pA, sw, Ol, n, oY, jX, nA, eW, br, h, dL, bl, aUH, Vb, Nr, sq, po, aUP, kp, aMJ, SE, se, Vy, hL, at, uz, oh, acJ, it, B, agx, AB, eR, aU, jx, sM, aNG, v, j, C, AD, Eh, q, eA, aT, cy, yj, qk, af, ajU, Lu, s, oV, Y, bg, r, en, cl, t, kC, ck, BU, sS, ac, c, alC, aUJ, wl, dy, cw, BD, aDy, qb, aUM, Jy, Ay, bs, bS, aSw, agz, hF, vJ, qh, qV, lC, aUA, aDh, aUE, MG, ff, eH, cr, kD, yy, aUD, Ej, uF, aUD, Pd, BG, aUC, bz, DI, dr, Nq, gk, aUN, xZ, bE, ag, dX, Fw, hE, iu, y); #c1(CP) c2(NP_000087) c3(ceruloplasmin precursor) c4(1552, 1553, 1554) c5(1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650) c6(1651, 1652, 1653) c7(T, ea, aUT, e, aR, aE, aU, K, az, hl, uC, eY, nB, U, aUS, nw, m, mk, aUU, dD, aw, aj, aUX, rP, cd, b, gq, ep, DU, ey, A, dw, aLO, W, ky, aUZ, B, ek, mr, j, nS, eA, J, axa, yw, hb, rS, HC, yu, cy, bq, aUld, an, DX, af, eu, aUY, fO, en, t, nC, xw, f, bN, rR, c, aUW, hr, dy, aUR, aen, aya, rQ, ha, eq, alr, py, oW, aln, aUV, sj, ff, cr, yy, S, Z, es, nb, gZ, ei, i, y, alq, X, hv); #c1(CPA1) c2(NP_001859) c3(carboxypeptidase A1 preproprotein) c4(121743) c5(121744, 121745, 121746, 121747, 121748, 121749, 121750, 121751) c6( ) c7(ar, tg, s, aW, vf, aT, vg, K, Fg, afb); #c1(CPA2) c2(NP_001860) c3(carboxypeptidase A2 precursor) c4(121752) c5(121753, 121754, 121755, 121756, 121757, 121758) c6( ) c7(bN); #c1(CPA3) c2(NP_001861) c3(mast cell carboxypeptidase A precursor) c4(121759) c5(121760, 121761, 121762, 12/763, (21764, 121765) c6( ) c7(bM, T, ae, ad); #c1(CPA4) c2(NP_001156918) c3(carboxypeptidase A4 isoform 2 preproprotein) c4(62026, 62027) c5(62028, 62029, 62030, 62031, 62032, 62033, 62034, 62035, 62036, 62037, 62038, 62039) c6( ) c7(bM, T, LO, r, PQ, ae, q, ad); #c1(CPA4) c2(NP_057436) c3(carboxypeptidase A4 preproprotein) c4(190787) c5(190788, 190789, 190790, 190791, 190792, 190793) c6( ) c7(bM, T, LO, r, PQ, ae, q, ad); #c1(CPA6) c2(NP_065094) c3(carboxypeptidase A6 preproprotein) c4(203860, 203861, 203862) c5(203863, 203864, 203865, 203866, 203867, 203868, 203869, 203870, 203871, 203872, 203873, 203874, 203875) c6( ) c7(aVa, arH, ha, lr, EQ, aNy, aVb, il); #c1(CPA6) c2(XP_011515872) c3(carboxypeptidase AG isoform X2) c4( ) c5(308847, 308848, 308849, 308850, 308851) c6( ) c7(aVa, arH, ha, lr, EQ, aNy, aVb, il); #c1(CPAMD8) c2(NP_056507) c3(C3 and PZP-like alpha-2-macroglobulin domain-containing protein 8) c4(187822, 187823) c5(187824, 187825, 187826, 187827, 187828, 187829, 187830, 187831, 187832, 187833, 187834, 187835, 187836, 187837, 187838, 187839) c6(187840, 187841, 187842, 187843) c7(ID, gh, gx, bB, V, ad, BY, aT, ji, ae, Qe, aW, hp, cm, bz, qa, c, eM, bE, vy, EY, y); #c1(CPAMD8) c2(XP_011526221) c3(C3 and PZP-like alpha-2-macroglobulin domain-containing protein 8 isoform X3) c4(320480) c5( ) c6( ) c7(ID, gh, gx, bB, V, ad, BY, aT, ji, ae, Qe, aW, hp, cm, bz, qa, c, eM, bE, vy, EY, y); #c1(CPAMD8) c2(XP_011526226) c3(C3 and PZP-like alpha-2-macroglobulin domain-containing protein 8 isoform X5) c4( ) c5(320481, 320482, 320483, 320484, 320485, 320486, 320487, 320488) c6(320489, 320490) c7(ID, gh, gx, bB, V, ad, BY, aT, ji, ae, de, aW, hp, cm, bz, qa, c, eM, bE, vy, EY, y); #c1(CPB1) c2(NP_001862) c3(carboxypeptidase B preproprotein) c4(121766, 121767, 121768) c5(12/769, 121770, 121771, 121772, 121773, 121774, 121775, 121776, 121777, 121778, 121779, 121780, 121781, 121782, 121783, 121784, 121785, 121786, 121787, 121788, 121789, 121790, 121791, 121792, 121793, 121794, 121795, 121796) c6(121797, 121798, 121799, 121800, 121801, 121802) c7(y, wb, ai); #c1(CPB2) c2(NP_001265470) c3(carboxypeptidase B2 isoform 2 preproprotein) c4(98621) c5(98622, 98623, 98624, 98625, 98626, 98627) c6( ) c7(P, bL, T, Ve, jv, dD, eR, oe, e, DV, aw, cz, sM, eb, aj, v, j, eq, ez, ai, av, ft, rS, M, Yk, hH, adx, eu, Vb, OV, d, azS, kl, cH, anX, sq, DN, AQ, aeS, ff, kD, aq, pD, en, g, pT, t, cM, aVc, bz, uC, eK, sn, ac, wE, ag, dh, hr, qq, dX, aS, m, ei, wx, B, amL); #c1(CPB2) c2(NP_001863) c3(carboxypeptidase B2 isoform 1 preproprotein) c4(121803) c5(121804, 121805, 121806, 121807, 121808, 121809) c6( ) c7(P, bL, T, Ve, jv, dD, eR, oe, e, DV, aw, cz, sM, eb, aj, v, j, eq, ez, ai, av, ft, rS, M, Yk, hH, adx, eu, Vb, OV, d, azS, kl, cH, anX, sq, DN, AQ, aeS, ff, kD, aq, pD, en, g, pT, t, cM, aVc, bz, uC, eK, sn, ac, wE, ag, dh, hr, qq, dX, aS, m, ei, wx, B, aml); #c1(CPD) c2(NP_00186704) c3(carboxypeptidase D isoform 2) c4(78371) c5(78372, 78373, 78374, 78375, 78376, 78377) c6( ) c7(oO, ae, T, hV, aW, yk, XY, CT, aT, ci, fl, cm, aed, bz, ad, hX); #c1(CPE) c2(NP_001864) c3(carboxypeptidase E preproprotein) c4(121810, 121811) c5(121812, 121813, 121814, 121815, 121816, 121817, 121818, 121819, 121820, 121821, 121822, 121823) c6( ) c7(bL, T, dD, al, aj, v, j, eq, ml, q, J, eA, qC, mj, ft, pX, K, qM, dQ, af, eG, A, d, aVd, cW, r, lo, t, uR, m, B, amL); #c1 (CPEB1) c2(NP_001073001) c3(cytoplasmic polyadenylation element-binding protein 1 isoform 2) c4(35568) c5(35569, 35570, 35571, 35572, 35573) c6( ) c7(ck, T, d, gU, eM, cj, Ku, g, eA); #c1(CPEB1) c2(NP_001073003) c3(cytoplasmic polyadenylation element-binding protein 1 isoform 3) c4( ) c5(35574, 35575, 35576, 35577, 35578, 35579) c6( ) c7(ck, T, d, gU, eM, cj, Ku, g, eA); #c1(CPEB1) c2(NP_001275749) c3(cytoplasmic polyadenylation element-binding protein 1 isoform 4) c4( ) c5(107237, 107238, 107239, 107240, 107241) c6( ) c7(ck, T, d, gU, eM, cj, Ku, g, eA); #c1(CPEB1) c2(NP_085097) c3(cytoplasmic polyadenylation element-binding protein 1 isoform 1) c4(219270, 219271, 219272, 219273, 219274) c5(219275, 219276, 219277, 219278, 219279, 219280, 219281, 219282, 219283, 219284, 219285, 219286, 219287, 219288, 219289, 219290, 219291, 219292, 219293, 219294, 219295, 219296, 219297, 219298) c6( ) c7(ck, T, d, gU, eM, cj, Ku, g, eA); #c1(CPEB3) c2(NP_001171608) c3(cytoplasmic polyadenylation element-binding protein 3 isoform 2) c4(70772) c5(70773, 70774, 70775, 70776, 70777, 70778, 70779, 70780, 70781, 70782) c6( ) c7(fq); #c1(CPEB3) c2(NP_055727) c3(cytoplasmic polyadenylation element-binding protein 3 isoform 1) c4(183145) c5(183146, 183147, 183148, 183149, 183150, 183151) c6( ) c7(fq); #c1(CPEB3) c2(XP_011537825) c3(cytoplasmic polyadenylation element-binding protein 3 isoform X12) c4( ) c5(335329) c6( ) c7(fq); #c1(CPEB4) c2(NP_085130) c3(cytoplasmic polyadenylation element-binding protein 4) c4(219345) c5(219346, 219347, 219348, 219349, 219350, 219351) c6( ) c7(iR, aq, dt, B); #c1(CPED1) c2(NP_001099003) c3(cadherin-like and PC-esterase domain-containing protein 1 isoform 2 precursor) c4(41603) c5(41604, 41605, 41606, 41607, 41608, 41609, 41610, 41611, 41612, 41613, 41614, 41615) c6( ) c7(v, agk); #c1(CPED1) c2(NP_079189) c3(cadherin-like and PC-esterase domain-containing protein 1 isoform 1 precursor) c4(217401, 217402, 217403) c5(217404, 217405, 217406, 217407, 217408, 217409, 217410, 217411, 217412, 217413, 217414, 217415) c6( ) c7(v, agk); #c1(CPLX1) c2(NP_006642) c3(complexin-1) c4(166848) c5(166849, 166850, 166851, 166852, 166853, (66854) c6( ) c7(hH, bK); #c1(CPLX2) c2(XP_011532721) c3(complexin-2 isoform X1) c4(328742, 328743, 328744, 328745, 328746, 328747) c5(328748, 328749, 328750, 328751, 328752, 328753, 328754, 328755, 328756, 328757, 328758, 328759, 328760, 328761, 328762, 328763, 328764, 328765, 328766, 328767, 328768, 328769, 328770, 328771, 328772, 328773, 328774, 328775, 328776, 328777, 328778, 328779, 328780, 328781, 328782, 328783, 328784, 328785, 328786, 328787, 328788, 328789, 328790, 328791) c6( ) c7(ac, aw, bS, bK, ct, Z, aR); #c1(CPLX3) c2(NP_001025176) c3(complexin-3 precursor) c4(29405) c5(29406, 29407, 29408, 29409, 29410, 29411) c6( ) c7(oe, Gx); #c1(CPLX4) c2(NP_857637) c3(complexin-4) c4(255297) c5(255298, 255299, 255300, 255301, 255302, 255303) c6( ) c7(Gx); #c1(CPM) c2(NP_001005502) c3(carboxypeptidase M precursor) c4(21769, 21770, 21771) c5(21772, 21773, 21774, 21775, 21776, 21777, 21778, 21779, 21780, 21781, 21782, 21783, 21784, 21785, 21786, 21787, 21788, 21789, 21790, 21791, 21792, 21793, 21794, 21795, 21796, 21797, 21798, 21799, 21800, 21801) c6( ) c7(dP, iu, T, oe, au, hj, kf, aHs, iN, QC, cd); #c1(CPN1) c2(XP_011537601) c3 (carboxypeptidase N catalytic chain isoform X1) c4(335187) c5(335188) c6( ) c7(af, fc, U, Fb, K, ei, aVe, ai); #c1(CPN2) c2(NP_001278917) c3(carboxypeptidase N subunit 2 precursor) c4(110952) c5(110953, 110954, 110955, 110956, 110957, 110958, 110959, 110960, 110961, 110962, 110963, 110964, 110965, 110966) c6(110967, 110968, 110969, 110970) c7(uz, Fb); #c1(CPNE1) c2(NP_001185792) c3(copine-1 isoform c) c4(76411, 76412) c5(76413, 76414, 76415, 76416, 76417, 76418, 76419, 76420, 76421, 76422, 76423, 76424, 76425, 76426, 76427, 76428, 76429, 76430, 76431, 76432, 76433, 76434, 76435, 76436, 76437, 76438, 76439, 76440, 76441, 76442, 76443, 76444, 76445, 76446, 76447, 76448, 76449, 76450, 76451, 76452, 76453, 76454, 76455, 76456, 76457, 76458, 76459) c6( ) c7(pZ); #c1(CPNE1) c2(NP_003906) c3(copine-1 isoform b) c4(142051, 142052, 142053, 142054, 142055) c5(142056, 142057, 142058, 142059, 142060, 142061, 142062, 142063, 142064, 142065, 142066, 142067, 142068, 142069, 142070, 142071, 142072, 142073, 142074, 142075, 142076, 142077, 142078, 142079, 142080, 142081, 142082, 142083, 142084) c6( ) c7(pZ); #c1(CPNE1) c2(NP_690905) c3(copine-1 isoform a) c4(245061, 245062, 245063) c5( ) c6( ) c7(pZ); #c1(CPNE2) c2(NP_689940) c3(copine-2) c4(244408, 244409) c5(244410, 244411, 244412, 244413, 244414, 244415, 244416, 244417, 244418, 244419, 244420, 244421, 244422, 244423, 244424, 244425) c6( ) c7(uz); #c1(CPNE3) c2(NP_003900) c3(copine-3) c4(141961, 141962, 141963, 141964, 141965, 141966) c5(141967, 141968, 141969, 141970, 141971, 141972, 141973, 141974, 141975, 141976, 141977, 141978, 141979, 141980, 141981, 141982, 141983, 141984, 141985, 141986, 141987, 141988, 141989, 141990, 141991, 141992, 141993, 141994, 141995, 141996, 141997, 141998, 141999, 142000, 142001, 142002, 142003, 142004, 142005, 142006) c6( ) c7(t, T, bz, ad, eM); #c1(CPNE4) c2(NP_001276041) c3(copine-4 isoform 1) c4(107808, 107809, 107810) c5(107811, 107812, 107813, 107814, 107815, 107816, 107817, 107818, 107819, 107820, 107821, 107822, 107823, 107824, 107825, 107826, 107827, 107828, 107829, 107830) c6( ) c7(bM, fq, aw, EQ); #c1(CPNE4) c2(NP_570720) c3(copine-4 isoform 2) c4( ) c5(234231, 234232, 234233, 234234, 234235, 234236) c6( ) c7(bM, fq, aw, EQ); #c1(CPNE7) c2(NP_055242) c3(copine-7 isoform b) c4(180011) c5(180012, 180013, 180014, 180015, 180016, 180017) c6( ) c7(aW, aC, cd, aT); #c1(CPNE7) c2(NP_705900) c3(copine-7 isoform a) c4(246403) c5(246404, 246405, 246406, 246407, 246408, 246409) c6( ) c7(aW, aC, cd, aT); #c1(CPNE8) c2(NP_705898) c3(copine-8) c4(246396) c5(246397, 246398, 246399, 246400, 246401, 246402) c6( ) c7(oe, dt); #c1(CPO) c2(XP_011508929) c3(carboxypeptidase 0 isoform X1) c4(299484) c5(299485, 299486, 299487, 299488, 299489, 299490, 299491, 299492) c6(299493, 299494) c7(bM, T, aVf, fd, aUA, ae, ad); #c1(CPOX) c2(NP_000088) c3(oxygen-dependent coproporphyrinogen-Ill oxidase, mitochondrial precursor) c4(1654) c5(1655, 1656, 1657, 1658, 1659, 1660) c6( ) c7(cw, bM, T, aC, BD, aDy, sw, QW, fd, bB, n, aNG, aHv, j, aVi, q, ad, eA, iu, aE, aT, h, cy, bl, K, aJA, aVg, ae, af, ajU, Nr, s, Ow, aW, aVf, ff, fO, eH, aNq, r, Ej, po, kp, aB, en, nN, eF, t, kC, bz, dr, Nq, y, sS, aBr, SE, xZ, wO, bE, aVh, at, acJ, m, QV, it, B, ao); #c1(CPOX) c2(XP_011510739) c3(oxygen-dependent coproporphyrinogen-Ill oxidase, mitochondrial isoform X2) c4( ) c5(301157) c6( ) c7(cw, bM, T, aC, BD, aDy, sw, QW, fd, bB, n, aNG, aHv, j, aVi, q, ad, eA, iu, aE, aT, h, cy, bl, K, aUA, aVg, ae, af, ajU, Nr, s, Ow, aW, aVf, ff, fO, eH, aNq, r, Ej, po, kp, aB, en, nN, eF, t, kC, bz, dr, Nq, y, sS, aBr, SE, xZ, wO, bE, aVh, at, acJ, m, QV, it, B, ao); #c1(CPPED1) c2(NP_001092925) c3(serine/threonine-protein phosphatase CPPED1 isoform b) c4(39771) c5(39772, 39773, 39774, 39775, 39776, 39777) c6( ) c7(n, Y, dL, Nn, el); #c1(CPPED1) c2(NP_060810) c3(serine/threonine-protein phosphatase CPPED1 isoform a) c4(197938) c5(197939, 197940, 197941, 197942, 197943, 197944) c6( ) c7(n, Y, dL, Nn, el); #c1(CPQ) c2(NP_057218) c3(carboxypeptidase [preproprotein) c4(189537) c5(189538, 189539, 189540, 189541, 189542, 189543, 189544, 189545) c6( ) c7(T, mR, Wz, bE, asD, hl, EQ, aVj, cz, bB, aq, h, uu, ox, fr, zy, y, B, adz); #c1(CPSI) c2(NP_001116105) c3(carbamoyl-phosphate synthase [ammonia], mitochondrial isoform a precursor) c4(43427, 43428, 43429, 43430) c5(43431, 43432, 43433, 43434, 43435, 43436, 43437, 43438, 43439, 43440, 43441, 43442, 43443, 43444, 43445, 43446, 43447, 43448, 43449, 43450, 43451, 43452, 43453, 43454, 43455, 43456, 43457, 43458, 43459, 43460, 43461, 43462, 43463, 43464, 43465, 43466, 43467, 43468, 43469, 43470, 43471, 43472, 43473, 43474, 43475, 43476, 43477, 43478, 43479, 43480, 43481, 43482, 43483, 43484, 43485, 43486, 43487, 43488, 43489, 43490, 43491, 43492, 43493, 43494, 43495, 43496, 43497, 43498, 43499, 43500, 43501, 43502, 43503, 43504, 43505, 43506, 43507, 43508, 43509, 43510, 43511, 43512, 43513, 43514, 43515, 43516, 43517, 43518, 43519, 43520, 43521, 43522, 43523, 43524, 43525, 43526, 43527, 43528, 43529, 43530, 43531, 43532, 43533, 43534, 43535, 43536, 43537, 43538, 43539, 43540, 43541, 43542, 43543, 43544, 43545, 43546, 43547, 43548, 43549, 43550, 43551, 43552, 43553, 43554, 43555, 43556, 43557, 43558, 43559, 43560, 43561, 43562, 43563, 43564, 43565, 43566, 43567, 43568, 43569, 43570, 43571, 43572, 43573, 43574, 43575, 43576, 43577, 43578, 43579, 43580, 43581, 43582, 43583, 43584, 43585, 43586, 43587, 43588, 43589, 43590, 43591, 43592, 43593, 43594, 43595) c6( ) c7(eR, jp, ha, aip, cz, v, afD, md, acu, jo, bq, aiV, fc, fQ, fH, aq, xH, iR, f, wz, aUS, aS, lC, B); #c1 (CPSI) c2(NP_001116106) c3(carbamoyl-phosphate synthase [ammonia], mitochondrial isoform c) c4(43596) c5(43597, 43598, 43599, 43600, 43601, 43602) c6( ) c7(eR, jp, ha, aip, cz, v, afD, md, acu, jo, bq, aiV, fc, fQ, fH, aq, xH, iR, f, wz, aUS, aS, lC, B); #c1(CPSI) c2(NP_001866) c3(carbamoyl-phosphate synthase [ammonia], mitochondrial isoform b precursor) c4( ) c5(121824, 121825, 121826, 121827, 121828) c6( ) c7(eR, jp, ha, aip, cz, v, afD, md, acu, jo, bq, aiV, fc, fQ, fH, aq, xH, iR, f, wz, aUS, aS, lC, B); #c1(CPSI) c2(XP_011508942) c3(carbamoyl-phosphate synthase [ammonia], mitochondrial isoform X2) c4(299504, 299505) c5(299506, 299507, 299508, 299509, 299510, 299511, 299512, 299513, 299514, 299515, 299516) c6( ) c7(eR, jp, ha, aip, cz, v, afD, md, acu, jo, bq, aiV, fc, fQ, fH, aq, xH, iR, f, wz, aUS, aS, lC, B); #c1(CPSF1) c2(NP_037423) c3(cleavage and polyadenylation specificity factor subunit 1) c4(175481) c5(175482, 175483, 175484, 175485, 175486, 175487) c6( ) c7(aA); #c1(CPSF2) c2(NP_059133) c3(cleavage and polyadenylation specificity factor subunit 2) c4(193209, 193210) c5(193211, 193212, 193213, 193214, 193215, 193216, 193217, 193218, 193219, 193220, 193221, 193222) c6( ) c7(bL, T, hd, nM, nB); #c1(CPSF3) c2(NP_057291) c3(cleavage and polyadenylation specificity factor subunit 3) c4(189918, 189919, 189920) c5(189921, 189922, 189923, 189924, 189925, 189926, 189927, 189928, 189929, 189930, 189931, 189932, 189933) c6( ) c7(aVk); #c1(CPSF3) c2(XP_011508664) c3(cleavage and polyadenylation specificity factor subunit 3 isoform X2) c4( ) c5(299285, 299286, 299287, 299288, 299289) c6( ) c7(aVk); #c1(CPSF3L) c2(XP_011539949) c3(integrator complex subunit II isoform X1) c4(338302) c5( ) c6( ) c7(sO, aVk); #c1(CPSF3L) c2(XP_011539950) c3(integrator complex subunit II isoform X2) c4(338303) c5(338304, 338305) c6( ) c7(sO, aVk); #c1(CPSF4) c2(NP_001075028) c3(cleavage and polyadenylation specificity factor subunit 4 isoform 2) c4(37187) c5(37188, 37189, 37190, 37191, 37192, 37193) c6( ) c7(bz, iu, g); #c1(CPSF4) c2(NP_006684) c3(cleavage and polyadenylation specificity factor subunit 4 isoform 1) c4(167128) c5(167129, 167130, 167131, 167132, 167133, 167134) c6( ) c7(bz, iu, g); #c1(CPSF6) c2(NP_001287876) c3(cleavage and polyadenylation specificity factor subunit 6 isoform 2) c4(113104) c5(113105, 113106, 113107, 113108, 113109, 113110) c6( ) c7(h, ey); #c1(CPSF6) c2(NP_008938) c3(cleavage and polyadenylation specificity factor subunit 6 isoform 1) c4(169676, 169677) c5(169678, 169679, 169680, 169681, 169682, 169683, 169684, 169685, 169686, 169687, 169688, 169689) c6( ) c7(h, ey); #c1(CPSF7) c2(NP_001129512) c3(cleavage and polyadenylation specificity factor subunit 7 isoform 2) c4( ) c5(51813, 51814, 51815, 51816, 51817) c6( ) c7(aW, n, T, aT); #c1(CPSF7) c2(NP_001136037) c3(cleavage and polyadenylation specificity factor subunit 7 isoform 3) c4(54113, 54114) c5(54115, 54116, 54117, 54118, 54119, 54120, 54121, 54122, 54123, 54124, 54125, 54126, 54127, 54128, 54129, 54130, 54131, 54132) c6( ) c7(aW, n, T, aT); #c1(CPSF7) c2(NP_079087) c3(cleavage and polyadenylation specificity factor subunit 7 isoform 1) c4(216937, 216938) c5(216939, 216940, 216941, 216942, 216943, 216944, 216945) c6( ) c7(aW, n, T, aT); #c1(CPSF7) c2(XP_005274355) c3(cleavage and polyadenylation specificity factor subunit 7 isoform X3) c4(285056) c5(285057, 285058, 285059, 285060) c6( ) c7(aW, n, T, aT); #c1(CPSF7) c2(XP_011543559) c3(cleavage and polyadenylation specificity factor subunit 7 isoform X4) c4(342471, 342472) c5(342473, 342474, 342475, 342476, 342477, 342478, 342479, 342480, 342481, 342482, 342483, 342484) c6( ) c7(aW, n, T, aT); #c1(CPT1A) c2(XP_005273819) c3(carnitine 0-palmitoyltransferase 1, liver isoform isoform X1) c4(284835) c5(284836, 284837, 284838, 284839, 284840, 284841, 284842, 284843, 284844, 284845, 284846, 284847, 284848, 284849) c6( ) c7(bL, cZ, dD, eR, al, aj, v, eq, ci, J, F, aT, jy, dd, bq, af, d, nU, fc, dq, EZ, bw, bo, mx, Xu, it); #c1(CPT1A) c2(XP_005273820) c3(carnitine 0-palmitoyltransferase 1, liver isoform isoform X2) c4(284850) c5(284851, 284852, 284853, 284854, 284855, 284856, 284857, 284858, 284859, 284860) c6( ) c7(bL, cZ, dD, eR, al, aj, v, eq, ci, J, F, aT, jy, dd, bq, af, d, nU, fc, dq, EZ, bw, bo, mx, Xu, it); #c1(CPT1B) c2(NP_001138606) c3(carnitine 0-palmitoyltransferase 1, muscle isoform isoform c) c4(57162, 57163) c5(57164, 57165, 57166, 57167, 57168, 57169, 57170, 57171, 57172, 57173, 57174, 57175) c6( ) c7(bL, cf, cj, aaN, aq, v, j, ei, bq, af, alY); #c1(CPT1B) c2(NP_004368) c3(carnitine 0-palmitoyltransferase 1, muscle isoform isoform a) c4(145783, 145784, 145785) c5(145786, 145787, 145788, 145789, 145790, 145791, 145792, 145793, 145794, 145795, 145796, 145797, 145798, 145799, 145800, 145801, 145802, 145803, 145804, 145805, 145806, 145807, 145808, 145809, 145810, 145811, 145812, 145813, 145814, 145815) c6( ) c7(bL, cF, cj, aaN, aq, v, j, ei, bq, af, alY); #c1(CPT1C) c2(NP_001129524) c3(carnitine 0-palmitoyltransferase 1, brain isoform isoform 1) c4(51846) c5(51847, 51848, 51849, 51850, 51851, 51852) c6( ) c7(av, cZ, d, Y, fd, K, bX, dd); #c1(CPT1C) c2(NP_001186682) c3(carnitine 0-palmitoyltransferase 1, brain isoform isoform 2) c4(78331) c5(78332, 78333, 78334, 78335, 78336, 78337, 78338, 78339, 78340, 78341, 78342, 78343, 78344, 78345) c6( ) c7(av, cZ, d, Y, fd, K, bX, dd); #c1(CPT1C) c2(XP_006723072) c3(carnitine 0-palmitoyltransferase 1, brain isoform isoform X3) c4(295000) c5(295001, 295002, 295003, 295004, 295005, 295006) c6( ) c7(av, cZ, d, Y, fd, K, bX, dd); #c1(CPT1C) c2(XP_011524742) c3(carnitine 0-palmitoyltransferase 1, brain isoform isoform X2) c4(319208) c5(319209, 319210, 319211, 319212, 319213, 319214) c6( ) c7(av, cZ, d, Y, fd, K, bX, dd); #c1(CPVL) c2(XP_011513739) c3(probable serine carboxypeptidase CPVL isoform X1) c4(306056, 306057, 306058, 306059) c5(306060, 306061, 306062, 306063, 306064, 306065, 306066, 306067, 306068, 306069, 306070, 306071, 306072, 306073, 306074, 306075, 306076, 306077, 306078, 306079, 306080, 306081, 306082, 306083, 306084, 306085, 306086, 306087, 306088, 306089, 306090, 306091) c6( ) c7(bL, ck, BI, T, RB, aE, QO, aVl, ds, oe, if, aiM, vy, cU, h, lo, EA, qN, hH, fq, aMk); #c1(CPXCR1) c2(NP_001171700) c3(CPX chromosomal region candidate gene 1 protein) c4(71321) c5(71322, 71323, 71324, 71325, 71326, 71327, 71328, 71329, 71330, 71331) c6( ) c7(aA); #c1(CPZ) c2(NP_001014447) c3(carboxypeptidase Z isoform 1 precursor) c4(25943) c5(25944, 25945, 25946, 25947, 25948, 25949, 25950, 25951, 25952, 25953, 25954, 25955, 25956, 25957, 25958, 25959, 25960) c6(25961, 25962, 25963, 25964) c7(cm); #c1(CPZ) c2(NP_001014448) c3(carboxypeptidase Z isoform 3) c4(25965, 25966) c5(25967, 25968, 25969, 25970, 25971, 25972, 25973) c6(25974, 25975) c7(cm); #c1(CPZ) c2(NP_003643) c3(carboxypeptidase Z isoform 2 precursor) c4(139860) c5(139861, 139862, 139863, 139864, 139865, 139866, 139867, 139868) c6(139869, 139870) c7(cm); #c1(CRI) c2(NP_000564) c3(complement receptor type 1 isoform F precursor) c4(12688, 12689, 12690) c5(12691, 12692, 12693, 12694, 12695, 12696, 12697, 12698, 12699, 12700, 12701, 12702, 12703, 12704, 12705, 12706, 12707, 12708, 12709, 12710, 12711, 12712) c6( ) c7(bL, bM, dD, eR, ea, fG, e, al, hE, aw, cz, iW, eq, ci, atK, eh, aHj, F, ld, dV, aT, alr, h, dL, aVn, af, ul, A, aW, Y, wc, hl, eZ, dr, asE, ck, LB, W, qe, ig, hc, aVm, dy, fl, eg, aTW, bt, fh, y, vi); #c1(CR1) c2(NP_000642) c3(complement receptor type 1 isoform S precursor) c4(14142, 14143) c5(14144, 14145, 14146, 14147, 14148, 14149) c6( ) c7(bL, bM, dD, eR, ea, fG, e, al, hE, aw, cz, iW, eq, ci, atK, eh, aHj, F, ld, dV, aT, alr, h, dL, aVn, af, ul, A, aW, Y, wc, hl, eZ, dr, asE, ck, LB, W, qe, ig, hc, aVm, dy, fl, eg, aTW, bt, fh, y, vi); #c1(CRI) c2(XP_006711229) c3(complement receptor type 1 isoform X2) c4( ) c5(285796, 285797, 285798, 285799, 285800, 285801) c6( ) c7(bL, bM, dD, eR, ea, fG, e, al, hE, aw, cz, iW, eq, ci, atK, eh, aHj, F, ld, dV, aT, alr, h, dL, aVn, af, ul, A, aW, Y, wc, hl, eZ, dr, asE, ck, LB, W, qe, ig, hc, aVm, dy, fl, eg, aTW, bt, fh, y, vi); #c1(CR2) c2(NP_001006659) c3(complement receptor type 2 isoform 1 precursor) c4(22338) c5(22339, 22340, 22341, 22342, 22343, 22344) c6( ) c7(bL, T, fM, aVo, iW, eX, jh, cz, fd, aj, ci, aVp, V, ai, mC, J, aoH, mj, hW, aVq, ja, h, eV, an, zy, hM, ff, eZ, aq, lo, BG, aoD, en, op, Wt, ck, iQ, W, vA, cj, eo, fl, y, Ze); #c1(CR2) c2(NP_001868) c3(complement receptor type 2 isoform 2 precursor) c4(121829, 121830, 121831) c5(121832, 121833, 121834, 121835, 121836, 121837, 121838, 121839, 121840, 121841, 121842, 121843, 121844, 121845, 121846, 121847, 121848, 121849) c6( ) c7(bL, T, fM, aVo, iW, ex, jh, cz, fd, aj, ci, aVp, V, ai, mC, J, aoH, mj, hW, aVq, ja, h, eV, an, zy, hM, ff, eZ, aq, lo, BG, aoD, en, op, Wt, ck, iQ, W, vA, cj, eo, fl, y, Ze); #c1(CRABP1) c2(NP_004369) c3(cellular retinoic acid-binding protein 1) c4(145816) c5(145817, 145818, 145819, 145820, 145821, 145822) c6( ) c7(T, fr, ci, bd, q, mP, j, s, ff, r, aq, en, cm, ik, op, sS, nM, nB, AM, vA, y, bt, it); #c1(CRABP2) c2(NP_00186652) c3(cellular retinoic acid-binding protein 2) c4(78285, 78286) c5(78287, 78288, 78289, 78290, 78291, 78292, 78293, 78294, 78295, 78296, 78297, 78298, 78299, 78300, 78301, 78302) c6( ) c7(bL, T, ci, eA, er, ad, LL, ec, aT, ae, jf, cH, aW, oV, ff, kp, en, g, as, cm, cM, bz, GR, op, ik, Fi, vA, aRD, iu); #c1(CRACR2A) c2(NP_001138430) c3(EF-hand calcium-binding domain-containing protein 4B isoform a) c4(56675) c5(56676, 56677, 56678, 56679, 56680, 56681, 56682, 56683, 56684, 56685, 56686, 56687) c6( ) c7(aw, gU, fc, an); #c1(CRACR2A) c2(NP_116069) c3(EF-hand calcium-binding domain-containing protein 48 isoform c) c4(225318) c5(225319, 225320, 225321, 225322, 225323, 225324) c6( ) c7(aw, gU, fc, an); #c1(CRACR2A) c2(XP_011519338) c3(EF-hand calcium-binding domain-containing protein 48 isoform X1) c4(313988, 313989) c5( ) c6( ) c7(aw, gU, fc, an); #c1(CRACR2A) c2(XP_011519340) c3(EF-hand calcium-binding domain-containing protein 48 isoform X2) c4( ) c5(313990, 313991, 313992, 313993, 313994, 313995) c6( ) c7(aw, gU, fc, an); #c1(CRADD) c2(NP_003796) c3(death domain-containing protein CRADD) c4(140945, 140946) c5(140947, 140948, 140949, 140950, 140951, 140952, 140953, 140954, 140955, 140956) c6( ) c7(cF, aw, eH, aVr, fr, eF, fq); #c1(CRADD) c2(XP_005269268) c3(death domain-containing protein CRADD isoform X1) c4(282507) c5(282508, 282509, 282510, 282511, 282512, 282513, 282514, 282515, 282516, 282517, 282518, 282519) c6( ) c7(cF, aw, eH, aVr, fr, eF, fq); #c1(CRAMP1L) c2(NP_065876) c3(protein cramped-like) c4(206016, 206017, 206018) c5(206019, 206020, 206021, 206022, 206023, 206024, 206025, 206026, 206027, 206028, 206029, 206030, 206031, 206032, 206033, 206034, 206035, 206036, 206037, 206038, 206039, 206040, 206041) c6( ) c7(ac); #c1(CRAT) c2(NP_000746) c3(carnitine 0-acetyltransferase isoform 1 precursor) c4(15132, 15133) c5(15134, 15135, 15136, 15137, 15138, 15139, 15140, 15141, 15142, 15143, 15144, 15145) c6( ) c7(bM, T, lP, if, bB, Vd, ci, qj, aE, K, aW, CT, La, aoD, Ku, eF, cm, bw, aAW, qJ, eM, nB, m, nA, qN, cd, eW, ft, pe, cU, h, on, pi, A, aq, ej, iO, lX, eD, hR, at, B, bL, rf, Kh, AD, q, er, mj, cF, nv, aT, bK, an, lM, ae, s, Y, r, en, cl, t, qs, ik, hY, nM, iM, c, kB, ig, mf, jh, Hn, ad, hX, hd, ds, nJ, je, qt, ff, eH, eZ, bz, qa, bE, aS, be, ei, pC); #c1(CRB1) c2(NP_001180569) c3(protein crumbs homolog 1 isoform 2 precursor) c4(74260, 74261) c5(74262, 74263, 74264, 74265, 74266, 74267) c6( ) c7(bM, nx, v, Nh, ab, li, lR, aVs, aVu, nk, nC, aVt, vk, W, aVv, ajw, hR, nw, eQ, oQ, aVw, de); #c1(CRB1) c2(NP_001244894) c3(protein crumbs homolog 1 isoform 3) c4(92273) c5(92274, 92275, 92276, 92277, 92278, 92279) c6( ) c7(bM, nx, v, Nh, ab, li, lR, aVs, aVu, nk, nC, aVt, vk, W, aVv, ajw, hR, nw, eQ, oQ, aVw, de); #c1(CRB1) c2(NP_001244895) c3(protein crumbs homolog 1 isoform 4 precursor) c4(92280) c5(92281, 92282, 92283, 92284, 92285, 92286, 92287, 92288, 92289, 92290, 92291, 92292) c6( ) c7(bM, nx, v, Nh, ab, li, lR, aVs, aVu, nk, nC, aVt, vk, W, aVv, ajw, hR, nw, eQ, oQ, aVw, de); #c1(CRB1) c2(NP_957705) c3(protein crumbs homolog 1 isoform 1 precursor) c4(262008) c5(262009, 262010, 262011, 262012, 262013, 262014, 262015) c6( ) c7(bM, nx, v, Nh, ab, li, lR, aVs, aVu, nk, nC, aVt, vk, W, aVv, ajw, hR, nw, eQ, oQ, aVw, de); #c1(CRB1) c2(XP_011507668) c3(protein crumbs homolog 1 isoform X2) c4(297654) c5(297655, 297656, 297657, 297658, 297659) c6( ) c7(bM, nx, v, Nh, ab, li, lR, aVs, aVu, nk, nC, aVt, vk, W, aVv, ajw, hR, nw, eQ, oQ, aVw, de); #c1(CRB1) c2(XP_011507670) c3(protein crumbs homolog 1 isoform X4) c4(297660) c5(297661, 297662, 297663, 297664, 297665, 297666, 297667, 297668) c6( ) c7(bM, nx, v, Nh, ab, li, lR, aVs, aVu, nk, nC, aVt, vk, W, aVv, ajw, hR, nw, eQ, oQ, aVw, de); #c1(CRB1) c2(XP_011507671) c3(protein crumbs homolog 1 isoform X5) c4(297669, 297670) c5(297671, 297672, 297673, 297674, 297675, 297676, 297677, 297678, 297679, 297680) c6( ) c7(bM, nx, v, Nh, ab, li, lR, aVs, aVu, nk, nC, aVt, vk, W, aVv, ajw, hR, nw, eQ, oQ, aVw, de); #c1(CRB2) c2(NP_775960) c3(protein crumbs homolog 2 precursor) c4(250824, 250825) c5(250826, 250827, 250828, 250829, 250830, 250831, 250832, 250833, 250834, 250835, 250836, 250837) c6( ) c7(Na, nC, nx, Nc, Nh); #c1(CRB2) c2(XP_005251991) c3(protein crumbs homolog 2 isoform X3) c4(271411) c5(271412, 271413, 271414, 271415, 271416, 271417) c6( ) c7(Na, nC, nx, Nc, Nh); #c1(CRB3) c2(NP_777377) c3(protein crumbs homolog 3 isoform b precursor) c4(251115) c5(251116, 251117, 251118, 251119, 251120, 251121) c6( ) c7(T, MU); #c1(CRB3) c2(XP_005259737) c3(protein crumbs homolog 3 isoform X1) c4(276280) c5(276281, 276282, 276283, 276284, 276285, 276286, 276287, 276288, 276289, 276290) c6( ) c7(T, MU); #c1(CRBN) c2(NP_001166953) c3(protein cereblon isoform 2) c4(68725) c5(68726, 68727, 68728, 68729, 68730, 68731, 68732) c6( ) c7(aC, oe, fd, bK, nA, ae, ad, aVx); #c1(CRBN) c2(NP_057386) c3(protein cereblon isoform 1) c4(190505, 190506) c5(190507, 190508, 190509, 190510, 190511, 190512) c6( ) c7(aC, oe, fd, bK, nA, ae, ad, aVx); #c1(CRBN) c2(XP_005265259) c3(protein cereblon isoform X2) c4( ) c5(279450, 279451, 279452, 279453, 279454) c6( ) c7(aC, oe, fd, bK, nA, ae, ad, aVx); #c1(CRCP) c2(NP_001035737) c3(DNA-directed RNA polymerase Ill subunit RPC9 isoform b) c4(32992, 32993) c5(32994, 32995, 32996, 32997, 32998, 32999, 33000, 33001, 33002, 33003, 33004) c6( ) c7(h, aW, My, aT); #c1(CRCP) c2(NP_001035738) c3(DNA-directed RNA polymerase III subunit RPC9 isoform c) c4(33005) c5(33006, 33007, 33008, 33009, 33010) c6( ) c7(h, aW, My, aT); #c1(CRCP) c2(NP_001135886) c3(DNA-directed RNA polymerase Ill subunit RPC9 isoform d) c4(53490) c5(53491, 53492, 53493, 53494, 53495, 53496, 53497, 53498) c6( ) c7(h, aW, My, aT); #c1(CRCP) c2(NP_055293) c3(DNA-directed RNA polymerase III subunit RPC9 isoform a) c4(180329) c5(180330, 180331, 180332, 180333, 180334, 180335) c6( ) c7(h, aW, My, aT); #c1(CRCT1) c2(NP_061933) c3(cysteine-rich C-terminal protein 1) c4(201209) c5(201210, 201211, 201212, 201213, 201214, 201215) c6( ) c7(bM); #c1(CREB1) c2(XP_011508947) c3(cyclic AMP-responsive element-binding protein 1 isoform X1) c4(299517, 299518, 299519) c5(299520, 299521, 299522, 299523, 299524, 299525, 299526, 299527) c6( ) c7(bM, T, ea, bO, fd, ci, aR, rw, K, cp, Jl, aW, Ls, fc, cm, bw, iT, xD, m, dD, iB, aw, aj, ct, eW, adf, aGR, ft, kH, h, ask, abf, aq, hq, ok, cA, hR, eo, B, bL, aGn, fG, aA, v, V, q, eA, di, aT, kf, cy, dz, bK, jj, ae, af, eu, ak, s, Y, r, cu, lo, t, f, FT, ck, Qi, acW, Dy, iM, gX, Yz, bt, hS, iW, ha, jh, bS, dG, eq, aVy, hF, ad, hX, av, db, ds, pq, d, je, aCt, eZ, aF, Z, adj, iR, bz, aU, ek, Ew, bE, dJ, rH, y); #c1(CREB1) c2(XP_011508948) c3(cyclic AMP-responsive element-binding protein 1 isoform X3) c4(299528) c5( ) c6( ) c7(bM, T, ea, bO, fd, ci, aR, rw, K, cp, JI, aW, Ls, fc, cm, bw, iT, xD, m, dD, iB, aw, aj, ct, eW, adf, aGR, ft, kH, h, ask, abf, aq, hq, ok, cA, hR, eo, B, bL, aGn, fG, aA, v, V, q, eA, di, aT, kf, cy, dz, bK, jj, ae, af, eu, ak, s, Y, r, cu, lo, t, f, FT, ck, Qi, acW, Dy, iM, gX, Yz, bt, hS, iW, ha, jh, bS, dG, eq, aVy, hF, ad, hX, av, db, ds, pq, d, je, aCt, eZ, aF, Z, adj, iR, bz, aU, ek, Ew, bE, dJ, rH, y); #c1(CREB1) c2(XP_011508949) c3(cyclic AMP-responsive element-binding protein 1 isoform X2) c4(299529, 299530) c5(299531, 299532, 299533, 299534, 299535, 299536, 299537, 299538, 299539, 299540, 299541, 299542, 299543, 299544, 299545, 299546, 299547) c6( ) c7(bM, T, ea, bO, fd, ci, aR, rw, K, cp, JI, aW, Ls, fc, cm, bw, iT, xD, m, dD, iB, aw, aj, ct, eW, adf, aGR, ft, KH, h, ask, abf, aq, hq, ok, cA, hR, eo, B, bL, aGn, fG, aA, v, V, q, eA, di, aT, kf, cy, dz, bK, jj, ae, af, eu, ak, s, Y, r, cu, lo, t, f, FT, ck, Qi, acW, Dy, iM, gX, Yz, bt, hS, iW, ha, jh, bS, dG, eq, aVy, hF, ad, hX, av, db, ds, pq, d, je, aCt, eZ, aF, Z, adj, iR, bz, aU, ek, Ew, bE, dJ, rH, y); #c1(CREB1) c2(XP_011508954) c3(cyclic AMP-responsive element-binding protein 1 isoform X7) c4(299548, 299549) c5(299550, 299551, 299552, 299553, 299554, 299555, 299556, 299557, 299558, 299559, 299560, 299561, 299562, 299563, 299564, 299565, 299566, 299567, 299568, 299569, 299570, 299571, 299572, 299573, 299574, 299575, 299576, 299577, 299578) c6( ) c7(bM, T, ea, bO, fd, ci, aR, rw, K, cp, Jl, aW, Ls, fc, cm, bw, iT, xD, m, dD, iB, aw, aj, ct, eW, adf, aGR, ft, kH, h, ask, abf, aq, hq, ok, cA, hR, eo, B, bL, aGn, fG, aA, v, V, q, eA, di, aT, kf, cy, dz, bK, jj, ae, af, eu, ak, s, Y, r, cu, lo, t, f, FT, ck, Qi, acW, Dy, iM, gX, Yz, bt, hS, iW, ha, jh, bS, dG, eq, aVy, hF, ad, hX, av, db, ds, pq, d, je, aCt, eZ, aF, Z, adj, iR, bz, aU, ek, Ew, bE, dJ, rH, y); #c1(CREB1) c2(XP_011508955) c3(cyclic AMP-responsive element-binding protein 1 isoform X8) c4(299579) c5(299580, 299581, 299582, 299583, 299584) c6( ) c7(bM, T, ea, bO, fd, ci, aR, rw, K, cp, JI, aW, Ls, fc, cm, bw, iT, xD, m, dD, iB, aw, aj, ct, eW, adf, aGR, ft, kH, h, ask, abf, aq, hq, ok, cA, hR, eo, B, bL, aGn, fG, aA, v, V, q, eA, di, aT, kf, cy, dz, bK, jj, ae, af, eu, ak, s, Y, r, cu, lo, t, f, FT, ck, Qi, acW, Dy, iM, gX, Yz, bt, hS, iW, ha, jh, bS, dG, eq, aVy, hF, ad, hX, av, db, ds, pq, d, je, aCt, eZ, aF, Z, adj, iR, bz, aU, ek, Ew, bE, dJ, rH, y); #c1(CREB3) c2(NP_006359) c3(cyclic AMP-responsive element-binding protein 3) c4(164337) c5(164338, 164339, 164340, 164341, 164342, 164343) c6( ) c7(dD, kj, aE, xD, aW, ea, zs, jx, aT, eq, y, af, MG); #c1(CREB3LI) c2(NP_443086) c3(cyclic AMP-responsive element-binding protein 3-like protein 1) c4(229323, 229324) c5(229325, 229326, 229327, 229328, 229329, 229330, 229331, 229332, 229333, 229334, 229335, 229336, 229337, 229338, 229339, 229340, 229341) c6( ) c7(eG, aw, zp, K, eA, aVz); #c1(CREB3L2) c2(NP_001240704) c3(cyclic AMP-responsive element-binding protein 3-like protein 2 isoform 2) c4(88704) c5(88705, 88706, 88707, 88708, 88709, 88710, 88711, 88712, 88713, 88714, 88715, 88716, 88717, 88718, 88719, 88720, 88721) c6( ) c7(T, hd, eG, ac, QG, nB, ci, QA, eA); #c1(CREB3L2) c2(NP_919047) c3(cyclic AMP-responsive element-binding protein 3-like protein 2 isoform 1) c4(258229, 258230, 258231, 258232) c5(258233, 258234, 258235, 258236, 258237, 258238, 258239, 258240, 258241, 258242, 258243, 258244, 258245, 258246) c6( ) c7(T, hd, eG, ac, QG, nB, ci, QA, eA); #c1(CREB3L3) c2(NP_001258924) c3(cyclic AMP-responsive element-binding protein 3-like protein 3 isoform b) c4(96800) c5(96801, 96802, 96803, 96804, 96805, 96806) c6( ) c7(aq, B, K, s); #c1(CREB3L3) c2(NP_115996) c3(cyclic AMP-responsive element-binding protein 3-like protein 3 isoform a) c4(225155, 225156) c5(225157, 225158, 225159, 225160, 225161, 225162, 225163, 225164, 225165, 225166, 225167, 225168, 225169, 225170, 225171) c6( ) c7(aq, B, K, s); #c1(CREB3L4) c2(NP_001242908) c3(cyclic AMP-responsive element-binding protein 3-like protein 4 isoform 1) c4(89211, 89212, 89213) c5(89214, 89215, 89216, 89217, 89218, 89219, 89220, 89221, 89222, 89223, 89224, 89225, 89226, 89227, 89228, 89229, 89230, 89231, 89232, 89233, 89234, 89235, 89236, 89237, 89238, 89239, 89240) c6( ) c7(JI, aW, qd, aT, K, g, ae, ad); #c1(CREB3L4) c2(XP_006711235) c3(cyclic AMP-responsive element-binding protein 3-like protein 4 isoform X1) c4(285802, 285803, 285804) c5(285805, 285806, 285807, 285808, 285809, 285810, 285811, 285812, 285813, 285814, 285815, 285816, 285817, 285818, 285819, 285820, 285821, 285822) c6( ) c7(JI, aW, qd, aT, K, g, ae, ad); #c1(CREB5) c2(NP_001011666) c3(cyclic AMP-responsive element-binding protein 5 isoform delta) c4(24675) c5(24676, 24677, 24678, 24679, 24680, 24681, 24682) c6( ) c7(bM, oe, ad, cz); #c1(CREB5) c2(NP_878901) c3(cyclic AMP-responsive element-binding protein 5 isoform alpha) c4(257125, 257126, 257127, 257128) c5(257129, 257130, 257131, 257132, 257133, 257134, 257135, 257136, 257137) c6( ) c7(bM, oe, ad, cz); #c1(CREB5) c2(XP_011513918) c3(cyclic AMP-responsive element-binding protein 5 isoform X4) c4( ) c5(306336, 306337, 306338, 306339, 306340, 306341, 306342, 306343, 306344, 306345, 306346, 306347, 306348, 306349, 306350, 306351, 306352, 306353, 306354, 306355) c6( ) c7(bM, oe, ad, cz); #c1(CREBBP) c2(NP_001073315) c3(CREB-binding protein isoform b) c4(35844, 35845) c5( ) c6( ) c7(bM, T, bO, fd, bB, ci, aR, x, aVC, aVA, dV, aVB, kS, aW, apn, cm, bw, aVE, eM, lp, aep, U, m, n, oY, nA, PJ, aCi, ft, h, dl, ask, he, pi, hi, avP, aVD, ok, hL, at, fL, bD, it, L, aTl, Kh, C, AO, q, eA, Kx, MR, jZ, qK, aT, bK, ae, af, jf, iU, iZ, r, en, cl, t, ck, Qi, nM, iM, c, kB, CZ, bt, ao, id, jh, hF, gb, mC, ad, hX, av, je, ff, eZ, adj, bz, dr, aU, bE, Kj, y, xg); #c1(CREBBP) c2(NP_004371) c3(CREB-binding protein isoform a) c4(145823) c5(145824, 145825, 145826, 145827, 145828, 145829, 145830, 145831, 145832, 145833, 145834, 145835, 145836, 145837, 145838, 145839, 145840, 145841, 145842) c6( ) c7(bM, T, bO, fd, bB, ci, aR, x, aVC, aVA, dV, aVB, kS, aW, apn, cm, bw, aVE, eM, lp, aep, U, m, n, oY, nA, PJ, aCi, ft, h, dl, ask, he, pi, hi, avP, aVD, ok, hL, at, fL, bD, it, L, aTl, Kh, C, AO, q, eA, Kx, MR, jZ, qK, aT, bK, ae, af, jf, iU, iZ, r, en, cl, t, ck, Qi, nM, iM, c, kB, CZ, bt, ao, id, jh, hF, gb, mC, ad, hX, av, je, ff, eZ, adj, bz, dr, aU, bE, Kj, y, xg); #c1(CREBBP) c2(XP_005255182) c3(CREB-binding protein isoform X4) c4(273571) c5( ) c6( ) c7(bM, T, bO, fd, bB, ci, aR, x, aVC, aVA, dV, aVB, kS, aW, apn, cm, bw, aVE, eM, lp, aep, U, m, n, oY, nA, PJ, aCi, ft, h, dL, ask, he, pi, hi, avP, aVD, ok, hL, at, FL, bD, it, L, aTl, Kh, C, AO, q, eA, Kx, MR, jZ, qK, aT, bK, ae, af, jf, iU, iZ, r, en, cl, t, ck, Qi, nM, iM, c, KB, CZ, bt, ao, id, jh, hF, gb, mC, ad, hX, av, je, ff, eZ, adj, bz, dr, aU, bE, Kj, y, xg); #c1(CREBBP) c2(XP_011520683) c3(CREB-binding protein isoform X5) c4( ) c5(315608, 315609, 315610, 315611, 315612, 315613, 315614, 315615) c6( ) c7(bM, T, bO, fd, bB, ci, aR, x, aVC, aVA, dV, aVB, kS, aW, apn, cm, bw, aVE, eM, lp, aep, U, m, n, oY, nA, PJ, aCi, ft, h, dl, ask, he, pi, hi, avP, aVD, ok, hL, at, FL, bD, it, L, aTl, Kh, C, AO, q, eA, Kx, MR, jZ, qK, aT, bK, ae, af, jf, iU, iZ, r, en, cl, t, ck, Qi, nM, iM, c, kB, CZ, bt, ao, id, jh, hF, gb, mC, ad, hX, av, je, ff, eZ, adj, bz, dr, aU, bE, Kj, y, xg); #c1(CREBBP) c2(XP_011520684) c3(CREB-binding protein isoform X6) c4( ) c5(315616) c6( ) c7(bM, T, bO, fd, bB, ci, aR, x, aVC, aVA, dV, aVB, KS, aW, apn, cm, bw, aVE, eM, lp, aep, U, m, n, oY, nA, PJ, aCi, ft, h, dl, ask, he, pi, hi, avP, aVD, ok, hL, at, fL, bD, it, L, aTl, Kh, C, AO, q, eA, Kx, MR, jZ, qK, aT, bK, ae, af, jf, iU, iZ, r, en, cl, t, ck, Qi, nM, iM, c, kB, CZ, bt, ao, id, jh, hF, gb, mC, ad, hX, av, je, ff, eZ, adj, bz, dr, aU, bE, Kj, y, xg); #c1 (CREBRF) c2(NP_001161865) c3(CREB3 regulatory factor isoform 2) c4(66211, 66212) c5(66213, 66214, 66215, 66216, 66217, 66218, 66219, 66220, 66221, 66222, 66223) c6( ) c7(ck, x, T, kh, aVF, cj, ci, k, y, pd, ae, Pm, ad); #c1 (CREBRF) c2(XP_011532743) c3(CREB3 regulatory factor isoform X1) c4(328798, 328799, 328800, 328801) c5(328802, 328803, 328804, 328805, 328806, 328807, 328808, 328809, 328810, 328811, 328812, 328813, 328814, 328815, 328816, 328817, 328818, 328819, 328820, 328821, 328822, 328823, 328824, 328825, 328826, 328827, 328828, 328829, 328830) c6( ) c7(ck, x, T, kh, aVF, cj, ci, k, y, pd, ae, Pm, ad); #c1(CREBZF) c2(NP_001034707) c3(CREB/ATF bZIP transcription factor) c4(32085, 32086) c5(32087, 32088, 32089, 32090, 32091, 32092, 32093, 32094, 32095, 32096, 32097, 32098, 32099, 32100, 32101, 32102, 32103) c6( ) c7(xD, gV, jf, jh, hG); #c1(CREG1) c2(NP_003842) c3(protein CREG1 precursor) c4(141309) c5(141310, 141311, 141312, 141313, 141314, 141315, 141316, 141317, 141318) c6(141319, 141320) c7(eG, dD, mR, ea, ajv, cz, hl, Hn, j, ci, eq, y, ad, bA); #c1(CRELD1) c2(NP_001026887) c3(cysteine-rich with EGF-like domain protein 1 isoform 1 precursor) c4(29854) c5(29855, 29856, 29857, 29858, 29859, 29860) c6( ) c7(aVJ, gY, aVG, bD, j, aVl, sx, aVH, f); #c1(CRELD1) c2(NP_056328) c3(cysteine-rich with EGF-like domain protein 1 isoform 2 precursor) c4(187100) c5(187101, 187102, 187103, 187104, 187105, 187106, 187107, 187108, 187109, 187110, 187111, 187112, 187113, 187114) c6( ) c7(aVJ, gY, aVG, bD, j, aVl, sx, aVH, f); #c1(CRELD2) c2(NP_001128573) c3(cysteine-rich with EGF-like domain protein 2 isoform a precursor) c4(50295) c5(50296, 50297, 50298, 50299, 50300, 50301, 50302, 50303) c6(50304, 50305) c7(jb, ry, ln, K); #c1(CRELD2) c2(NP_001271246) c3(cysteine-rich with EGF-like domain protein 2 isoform c precursor) c4(103731) c5(103732, 103733, 103734, 103735, 103736, 103737, 103738, 103739) c6(103740, 103741) c7(jb, ry, ln, K); #c1(CRELD2) c2(NP_001271247) c3(cysteine-rich with EGF-like domain protein 2 isoform d precursor) c4(103742) c5(103743, 103744, 103745, 103746, 103747, 103748, 103749, 103750) c6(103751, 103752) c7(jb, ry, ln, K); #c1(CRELD2) c2(NP_077300) c3(cysteine-rich with EGF-like domain protein 2 isoform b precursor) c4(215115) c5(215116, 215117, 215118, 215119, 215120, 215121, 215122, 215123) c6(215124, 215125) c7(jb, ry, ln, K); #c1(CREM) c2(NP_001254491) c3(CAMP-responsive element modulator isoform 23) c4( ) c5(94519, 94520, 94521, 94522, 94523, 94524) c6( ) c7(bL, gU, aw, b, fd, dG, v, ww, aVK, mC, ad, hd, ds, vH, bK, K, an, he, ae, af, yo, A, tb, ND, Y, fJ, eZ, vs, g, hq, nM, nB, NI, fL, it, AN); #c1(CREM) c2(NP_001254493) c3(CAMP-responsive element modulator isoform 25) c4(94525) c5(94526, 94527, 94528, 94529, 94530, 94531) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, fL, it, AN); #c1(CREM) c2(NP_001254499) c3(CAMP-responsive element modulator isoform 31) c4( ) c5(94532) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, fL, it, AN); #c1(CREM) c2(NP_001872) c3(cAMP-responsive element modulator isoform 2) c4(121859) c5(121860, 121861, 121862, 121863, 121864, 121865, 121866, 121867, 121868, 121869, 121870) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, FL, it, AN); #c1(CREM) c2(NP_874386) c3(CAMP-responsive element modulator isoform 4) c4(256662) c5(256663, 256664, 256665, 256666, 256667, 256668, 256669) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, fL, it, AN); #c1(CREM) c2(NP_874387) c3(CAMP-responsive element modulator isoform 5) c4(256670) c5(256671, 256672, 256673, 256674, 256675, 256676, 256677, 256678, 256679, 256680, 256681) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, FL, it, AN); #c1(CREM) c2(NP_874388) c3(CAMP-responsive element modulator isoform 6) c4(256682) c5(256683, 256684, 256685, 256686, 256687, 256688) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, fL, it, AN); #c1(CREM) c2(NP_874389) c3(CAMP-responsive element modulator isoform 7) c4(256689) c5(256690, 256691, 256692, 256693, 256694, 256695) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, fL, it, AN); #c1(CREM) c2(NP_874390) c3(CAMP-responsive element modulator isoform 8) c4(256696) c5(256697, 256698, 256699, 256700, 256701, 256702) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, FL, it, AN); #c1(CREM) c2(NP_874392) c3(CAMP-responsive element modulator isoform 10) c4( ) c5(256703, 256704, 256705, 256706, 256707, 256708, 256709, 256710, 256711, 256712, 256713, 256714, 256715, 256716, 256717) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, fL, it, AN); #c1(CREM) c2(NP_874393) c3(cAMP-responsive element modulator isoform (1) c4(256718) c5(256719, 256720, 256721, 256722, 256723, 256724) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, FL, it, AN); #c1(CREM) c2(NP_877570) c3(CAMP-responsive element modulator isoform 13) c4( ) c5(256892, 256893, 256894, 256895, 256896, 256897) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, FL, it, AN); #c1(CREM) c2(NP_877572) c3(CAMP-responsive element modulator isoform 15) c4(256898) c5(256899, 256900, 256901, 256902, 256903, 256904) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, fL, it, AN); #c1(CREM) c2(NP_877573) c3(cAMP-responsive element modulator isoform 16) c4(256905) c5(256906, 256907, 256908, 256909, 256910, 256911) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, FL, it, AN); #c1(CREM) c2(NP_898831) c3(cAMP-responsive element modulator isoform 19) c4(257762, 257763) c5(257764, 257765, 257766, 257767, 257768, 257769, 257770, 257771, 257772, 257773, 257774, 257775) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, fL, it, AN); #c1(CREM) c2(NP_898883) c3(CAMP-responsive element modulator isoform 22) c4( ) c5(257822, 257823, 257824, 257825, 257826, 257827, 257828, 257829, 257830, 257831, 257832, 257833, 257834, 257835, 257836) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, FL, it, AN); #c1(CREM) c2(XP_006717441) c3 (CAMP-responsive element modulator isoform X3) c4(291114, 291115, 29/116) c5(29/117, 291118, 291119, 291120, 291121, 291122) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, FL, it, AN); #c1(CREM) c2(XP_006717442) c3(CAMP-responsive element modulator isoform X1) c4(291123) c5(291124, 291125, 291126, 291127, 291128, 291129, 291130, 291131, 291132, 291133, 291134, 291135) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, FL, it, AN); #c1(CREM) c2(XP_006717445) c3(cAMP-responsive element modulator isoform X18) c4(291136) c5(291137) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, fL, it, AN); #c1(CREM) c2(XP_006717446) c3(CAMP-responsive element modulator isoform X2) c4(291138) c5(291139, 291140, 291141, 291142, 291143, 291144) c6( ) c7(bL, gU, aw, bD, fd, dG, v, ww, aVK, mC, ad, hd, ds, vH, bK, K, an, he, ae, af, yo, A, tb, ND, Y, fJ, eZ, vs, g, hq, nM, nB, NI, FL, it, AN); #c1(CREM) c2(XP_011517619) c3(CAMP-responsive element modulator isoform X5) c4(311409, 311410) c5(311411, 311412, 311413, 311414, 311415, 311416) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, fL, it, AN); #c1(CREM) c2(XP_011517620) c3(CAMP-responsive element modulator isoform X6) c4(3114/7) c5( ) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, FL, it, AN); #c1(CREM) c2(XP_011517621) c3(CAMP-responsive element modulator isoform X7) c4(311418, 311419) c5(311420, 311421, 311422, 311423, 311424, 311425) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, FL, it, AN); #c1(CREM) c2(XP_011517622) c3(CAMP-responsive element modulator isoform X8) c4(311426, 311427, 311428) c5(311429, 311430, 311431, 311432, 311433, 311434, 311435, 311436, 311437, 311438, 311439, 311440) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, FL, it, AN); #c1(CREM) c2(XP_011517624) c3(CAMP-responsive element modulator isoform X9) c4(311441, 311442) c5(311443, 311444, 311445, 311446, 311447, 311448) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, fL, it, AN); #c1(CREM) c2(XP_011517627) c3(CAMP-responsive element modulator isoform X11) c4(311449) c5(311450, 311451, 311452, 311453, 311454, 311455) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, fL, it, AN); #c1(CREM) c2(XP_011517630) c3(CAMP-responsive element modulator isoform X14) c4(311456) c5(311457, 311458, 311459, 311460, 311461, 311462) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, FL, it, AN); #c1 (CREM) c2(XP_011517631) c3(CAMP-responsive element modulator isoform X15) c4(311463, 311464) c5(311465, 311466, 311467, 311468, 311469, 311470, 311471, 311472, 311473, 311474, 311475, 311476) c6( ) c7(bL, gU, aw, bO, fd, dG, v, ww, aVK, mC, ad, hd, ds, vH, bK, K, an, he, ae, af, yo, A, tb, ND, Y, fJ, eZ, vs, g, hq, nM, nB, NI, FL, it, AN); #c1(CRH) c2(NP_000747) c3(corticoliberin preproprotein) c4(15146) c5(15147, 15148, 15149, 15150, 15151, 15152, 15153, 15154) c6(15155, 15156) c7(bM, T, fR, vf, if, bO, eb, cL, gf, ci, aY, rw, iX, pX, aU, abj, K, aVL, yo, aW, aVQ, Fe, az, aVO, eE, cm, uC, mZ, uo, uJ, aVN, m, xh, sQ, My, n, To, akA, aj, ct, ml, qN, ww, ai, ke, ft, Nn, aMD, adX, h, eV, mV, mY, he, cC, mU, apo, aq, Hu, mX, hq, amt, ok, bP, cA, adZ, bo, Ei, vL, hR, ut, Pr, B, mT, aNW, cB, aNX, jv, eR, eX, aee, CR, mS, Nt, v, j, C, il, J, gM, aVM, aql, aT, bk, bq, hK, ae, af, eu, tN, ls, s, Y, fO, cu, en, cl, t, xw, aff, rR, ac, aeM, gx, wi, akd, aVP, aD, Gd, bS, aVy, ad, wD, dP, fU, oz, mW, lQ, cx, nJ, vO, hZ, aga, Di, Gv, ff, cr, aFr, iV, Z, iR, atM, xZ, pk, y, ra); #c1(CRHBP) c2(NP_001873) c3(corticotropin-releasing factor-binding protein precursor) c4(121871) c5(121872, 121873, 121874, 121875, 121876, 121877) c6( ) c7(jv, fR, sQ, bO, cz, Gd, bS, gf, ct, v, il, ke, aVM, bK, K, lQ, an, he, aFr, cu, Z, uC, hq, es, ac, cA, iA, vL, uo, ut); #c1(CRHR1) c2(NP_001138618) c3(corticotropin-releasing factor receptor 1 isoform 1 precursor) c4(57202) c5(57203, 57204, 57205, 57206, 57207, 57208, 57209, 57210, 57211, 57212) c6( ) c7(bM, T, FR, bO, gf, aVS, ip, x, azY, Gb, K, yo, amy, aW, mQ, Fa, Au, Ku, g, Jl, uC, Gs, U, uo, k, m, act, sd, aVR, n, To, ww, ct, pM, cU, he, abf, sp, hq, jq, ok, aqp, cA, bP, yP, Ei, vL, at, ut, bL, jv, eR, oe, cz, v, wy, V, mj, hb, aVM, aql, aT, bK, ae, bA, tN, s, fO, cu, t, FT, pH, vZ, sY, Gd, bS, OH, ad, dP, iC, LA, ji, lQ, tb, eZ, aFr, gl, iV, Z, pk, y, ra); #c1(CRHR1) c2(NP_001138619) c3 (corticotropin-releasing factor receptor 1 isoform 3 precursor) c4(57213, 57214) c5(57215, 57216, 57217, 57218, 57219, 57220, 57221, 57222, 57223, 57224) c6( ) c7(bM, T, FR, bO, gf, aVS, ip, x, azY, Gb, K, yo, amy, aW, mQ, Fa, Au, Ku, g, JI, uC, Gs, U, uo, k, m, act, sQ, aVR, n, To, ww, ct, pM, cU, he, abf, sp, hq, jq, ok, aqp, cA, bP, yP, Ei, vL, at, ut, bL, jv, eR, oe, cz, v, wy, V, mj, hb, aVM, aql, aT, bK, ae, bA, tN, s, fO, cu, t, FT, pH, vZ, sY, Gd, bS, OH, ad, dP, iC, LA, ji, IQ, tb, eZ, aFr, gl, iV, Z, pK, y, ra); #c1(CRHR1) c2(NP_001138620) c3(corticotropin-releasing factor receptor 1 isoform 4 precursor) c4(57225, 57226) c5(57227, 57228, 57229, 57230, 57231, 57232, 57233, 57234, 57235, 57236, 57237, 57238, 57239, 57240, 57241, 57242, 57243, 57244, 57245, 57246) c6( ) c7(bM, T, FR, bO, gf, aVS, ip, x, azY, Gb, K, yo, amy, aW, mQ, Fa, Au, Ku, g, JI, uC, Gs, U, uo, k, m, act, sQ, aVR, n, To, ww, ct, pM, cU, he, abf, sp, hq, jq, ok, aqp, cA, bP, yP, Ei, vL, at, ut, bL, jv, eR, oe, cz, v, wy, V, mj, hb, aVM, aql, aT, bK, ae, bA, tN, s, fO, cu, t, FT, pH, vZ, sY, Gd, bS, OH, ad, dP, iC, LA, ji, lQ, tb, eZ, aFr, gl, iV, Z, pk, y, ra); #c1(CRHR1) c2(NP_001289949) c3(corticotropin-releasing factor receptor 1 isoform 11-B) c4(114869) c5( ) c6( ) c7(bM, T, fR, b, gf, aVS, ip, x, azY, Gb, K, yo, amy, aW, ml, Fa, Au, Ku, g, JI, uC, Gs, U, uo, k, m, act, sQ, aVR, n, To, ww, ct, pM, cU, he, abf, sp, hq, jq, ok, aqp, cA, bP, yP, Ei, vl, at, ut, bL, jv, eR, oe, cz, v, wy, V, mj, hb, aVM, aql, aT, bK, ae, bA, tN, s, fO, cu, t, FT, pH, vZ, sY, Gd, bS, OH, ad, dP, iC, LA, ji, lQ, tb, eZ, aFr, gl, iV, Z, pK, y, ra); #c1 (CRHR1) c2(NP_004373) c3(corticotropin-releasing factor receptor 1 isoform 2 precursor) c4(145877) c5(145878, 145879, 145880, 145881, 145882, 145883, 145884, 145885, 145886, 145887, 145888, 145889, 145890, 145891) c6( ) c7(bM, T, FR, bO, gf, aVS, ip, x, azY, Gb, K, yo, amy, aW, mQ, Fa, Au, Ku, g, JI, uC, Gs, U, uo, k, m, act, sQ, aVR, n, To, ww, ct, pM, cU, he, abf, sp, hq, jq, ok, aqp, cA, bP, yP, Ei, vL, at, ut, bL, jv, eR, oe, cz, v, wy, V, mj, hb, aVM, aql, aT, bK, ae, bA, tN, s, fO, cu, t, FT, pH, vZ, sY, Gd, bS, OH, ad, dP, iC, LA, ji, lQ, tb, eZ, aFr, gl, iV, Z, pk, y, ra); #c1(CRHR2) c2(NP_001189404) c3(corticotropin-releasing factor receptor 2 isoform 2 precursor) c4(79366) c5(79367, 79368, 79369, 79370, 79371, 79372) c6( ) c7(bM, sY, T, FR, sQ, jx, bO, aA, cz, n, To, bS, v, ct, aVS, pM, fr, ww, wy, q, ad, dP, azY, py, aql, aT, cU, cy, bK, K, lQ, bl, ji, aa, abf, tb, s, aW, cr, r, aFr, tv, cu, aq, tu, aF, Z, uC, hq, es, ok, bP, cA, Ei, uo, ut, y, ra, B, amL); #c1(CRHR2) c2(NP_001189410) c3(corticotropin-releasing factor receptor 2 isoform 3) c4(79380) c5(79381, 79382, 79383, 79384, 79385, 79386) c6( ) c7(bM, sY, T, FR, sQ, jx, bO, aA, cz, n, To, bS, v, ct, aVS, pM, fr, ww, wy, q, ad, dP, azY, py, aql, aT, cU, cy, bK, K, lQ, bl, ji, aa, abf, tb, s, aW, cr, r, aFr, tv, cu, aq, tu, af, Z, uC, hq, es, ok, bP, cA, Ei, uo, ut, y, ra, B, amL); #c1(CRHR2) c2(NP_001874) c3 (corticotropin-releasing factor receptor 2 isoform 1) c4(121878) c5(121879, 121880, 121881, 121882, 121883, 121884) c6( ) c7(bM, sY, T, FR, sQ, jx, bO, aA, cz, n, To, bS, v, ct, aVS, pM, fr, ww, wy, q, ad, dP, azY, py, aql, aT, cU, cy, bK, K, lQ, bl, ji, aa, abf, tb, s, aW, cr, r, aFr, tv, cu, aq, tu, aF, Z, uC, hq, es, ok, bP, cA, Ei, uo, ut, y, ra, B, amL); #c1(CRHR2) c2(XP_011513430) c3(corticotropin-releasing factor receptor 2 isoform X3) c4(305689) c5(305690, 305691, 305692, 305693, 305694, 305695) c6( ) c7(bM, sY, T, fR, sQ, jx, bO, aA, cz, n, To, bS, v, ct, aVS, pM, fr, ww, wy, q, ad, dP, azY, py, aql, aT, cU, cy, bK, K, lQ, bl, ji, aa, abf, tb, s, aW, cr, r, aFr, tv, cu, aq, tu, aF, Z, uC, hq, es, ok, bP, cA, Ei, uo, ut, y, ra, B, amL); #c1(CRIM1) c2(NP_057525) c3(cysteine-rich motor neuron 1 protein precursor) c4(191246) c5(191247, 191248, 191249, 19/250, 191251, 191252) c6( ) c7(eW, bK); #c1(CRIP2) c2(NP_001257766) c3(cysteine-rich protein 2 isoform 2) c4(95625) c5(95626, 95627, 95628, 95629, 95630, 95631) c6( ) c7(aW, it, aT, afu); #c1(CRIP2) c2(NP_001257770) c3(cysteine-rich protein 2 isoform 3) c4(95632) c5(95633, 95634, 95635, 95636, 95637, 95638) c6( ) c7(aW, it, aT, afu); #c1(CRIP3) c2(NP_996805) c3(cysteine-rich protein 3) c4(264590, 264591) c5(264592, 264593, 264594, 264595, 264596, 264597, 264598, 264599, 264600, 264601, 264602, 264603, 264604) c6( ) c7(cc, ac); #c1(CRIP3) c2(XP_005249160) c3(cysteine-rich protein 3 isoform X1) c4(269049) c5(269050, 269051, 269052, 269053, 269054, 269055) c6( ) c7(cc, ac); #c1(CRIP3) c2(XP_011512911) c3(cysteine-rich protein 3 isoform X4) c4(304278) c5(304279, 304280, 304281, 304282, 304283) c6( ) c7(cc, ac); #c1(CRIP3) c2(XP_011512912) c3(cysteine-rich protein 3 isoform X5) c4(304284) c5( ) c6( ) c7(cc, ac); #c1(CRIPAK) c2(NP_787114) c3(cysteine-rich PAKI inhibitor) c4(252110, 252111) c5(252112, 252113, 252114, 252115, 252116, 252117, 252118, 252119, 252120, 252121, 252122, 252123, 252124, 252125, 252126) c6( ) c7(aW, aT); #c1(CRIPT) c2(NP_054890) c3(cysteine-rich PDZ-binding protein) c4(178220) c5(178221, 178222, 178223, 178224, 178225, 178226) c6( ) c7(avx, ayR); #c1(CRISPI) c2(NP_001122) c3(cysteine-rich secretory protein 1 isoform 1 precursor) c4(46810) c5( ) c6( ) c7(bM, K); #c1(CRISPI) c2(NP_733758) c3(cysteine-rich secretory protein 1 isoform 2 precursor) c4(246955) c5(246956, 246957, 246958, 246959, 246960, 246961, 246962, 246963, 246964, 246965, 246966, 246967, 246968) c6( ) c7(bM, K); #c1(CRISP2) c2(NP_001135879) c3(cysteine-rich secretory protein 2 precursor) c4(53483) c5(53484, 53485, 53486, 53487, 53488, 53489) c6( ) c7(T, gU, Tb, DD, aZ, if, e, aA, bB, fd, v, lR, ci, qN, fr, cd, ad, ai, eA, DJ, Dq, aT, cy, ae, cp, jf, asp, s, nO, aW, Y, oV, ff, aF, en, cl, dw, aaQ, cm, bz, pb, DT, eM, iA, bE, hL, agf, mf, m, y, iu); #c1(CRISP2) c2(XP_005249410) c3(cysteine-rich secretory protein 2 isoform X2) c4(269297) c5(269298, 269299, 269300, 269301, 269302, 269303) c6( ) c7(T, gU, Tb, DD, aZ, if, e, aA, bB, fd, v, lR, ci, qN, fr, cd, ad, ai, eA, DJ, Dq, aT, cy, ae, cp, jf, asp, s, nO, aW, Y, oV, ff, aF, en, cl, dw, aaQ, cm, bz, pb, DT, eM, iA, bE, hl, agf, mf, m, y, iu); #c1(CRISP3) c2(NP_001177915) c3(cysteine-rich secretory protein 3 isoform 2 precursor) c4(73139, 73140) c5(73141, 73142, 73143, 73144, 73145, 73146, 73147, 73148, 73149, 73150, 73151, 73152, 73153, 73154, 73155, 73156, 73157) c6(73158, 73159, 73160, 73161) c7(gx, T, fN, aA, n, Fb, aF, ci, g, ae, Vs, ad); #c1(CRISP3) c2(NP_006052) c3(cysteine-rich secretory protein 3 isoform 1 precursor) c4(161473, 161474, 161475) c5(161476, 161477, 161478, 161479, 161480, 161481, 161482, 161483, 161484, 161485, 161486, 161487, 161488, 161489, 161490, 161491, 161492, 161493, 161494, 161495, 161496, 161497, 161498) c6(161499, 161500, 161501, 161502, 161503, 161504) c7(gx, T, fN, aA, n, Fb, aF, ci, g, ae, Vs, ad); #c1 (CRISPLD1) c2(NP_001273707) c3(cysteine-rich secretory protein LCCL domain-containing 1 isoform 3) c4( ) c5(105686, 105687, 105688, 105689, 105690, 105691, 105692) c6(105693, 105694) c7(aaw, fq); #c1(CRISPLD1) c2(NP_113649) c3(cysteine-rich secretory protein LCCL domain-containing 1 isoform 1 precursor) c4(221925, 221926, 221927) c5(221928, 221929, 221930, 221931, 221932, 221933, 221934, 221935, 221936, 221937, 221938, 221939, 221940, 221941, 221942, 221943, 221944) c6(221945, 221946, 221947, 221948) c7(aaw, fq); #c1(CRISPLD2) c2(NP_113664) c3(cysteine-rich secretory protein LCCL domain-containing 2 precursor) c4(222010) c5(222011, 222012, 222013, 222014, 222015, 222016) c6( ) c7(Na, apM, hl, Nc, C, iq, aa); #c1(CRK) c2(NP_005197) c3(adapter molecule crk isoform b) c4(153819) c5(153820, 153821, 153822, 153823, 153824, 153825) c6( ) c7(T, aC, ea, if, fd, bB, gf, lR, Lw, bv, xE, aE, rw, dV, K, aW, FD, zC, g, as, eF, cm, bw, eM, cj, NO, Hb, m, NN, NK, dD, NJ, aj, BF, ct, qN, fr, cd, ai, Hx, b, jz, cU, h, fe, aq, NL, w, hR, B, bL, Dw, NO, fM, yk, lH, jx, aA, CR, cz, v, j, C, V, q, eA, eJ, yw, aT, kf, cy, ae, af, hM, wr, iU, Y, r, lo, en, t, iK, pb, bN, ck, im, bt, ao, kh, jh, dG, eq, iJ, ad, dP, av, CJ, F, ds, ji, lQ, NM, NP, d, ff, eH, cr, aF, bz, bE, i, y, iu); #c1(CRK) c2(NP_058431) c3(adapter molecule crk isoform a) c4(192695) c5(192696, 192697, 192698, 192699, 192700, 192701) c6( ) c7(T, aC, ea, if, fd, bB, gf, lR, Lw, bv, xE, aE, rw, dV, K, aW, FD, zC, g, as, eF, cm, bw, eM, cj, NO, Hb, m, NN, NK, dD, NJ, aj, BF, ct, qN, fr, cd, ai, Hx, b, jz, cU, h, fe, aq, NL, w, hR, B, bL, Dw, NO, fM, yk, lH, jx, aA, CR, cz, v, j, C, V, q, eA, eJ, yw, aT, kf, cy, ae, af, hM, wr, iU, Y, r, lo, en, t, iK, pb, bN, ck, im, bt, ao, kh, jh, dG, eq, iJ, ad, dP, av, CJ, F, ds, ji, lQ, NM, NP, d, ff, eH, cr, aF, bz, bE, i, y, iu); #c1 (CRKL) c2(NP_005198) c3(crk-like protein) c4(153826) c5(153827, 153828, 153829, 153830, 153831, 153832, 153833, 153834, 153835, 153836) c6( ) c7(T, RX, aA, ci, pd, ad, x, bv, ft, agd, aT, ae, iU, aW, aF, bz, bN, ck, eM, k, bD, y); #c1 (CRLF1) c2(NP_004741) c3(cytokine receptor-like factor 1 precursor) c4(149192) c5( ) c6( ) c7(LB, fG, aPh, aQH, amj, Nc, hE, aVT); #c1(CRLF1) c2(XP_011526725) c3(cytokine receptor-like factor 1 isoform X2) c4( ) c5(321039, 321040, 321041, 321042, 321043, 321044) c6( ) c7(LB, fG, aPh, aQH, amj, Nc, hE, aVT); #c1(CRLF2) c2(NP_071431) c3(cytokine receptor-like factor 2 isoform 1 precursor) c4(211622, 211623, 211624) c5(211625, 211626, 211627, 211628, 211629, 211630, 211631, 211632, 211633, 211634, 211635, 211636) c6( ) c7(ck, bM, ou, x, fX, eE, cj, zV, k, ox, aVU, ajo, f, lM, iL, Us); #c1(CRLF2) c2(XP_011543937) c3(cytokine receptor-like factor 2 isoform X2) c4( ) c5(343158, 343159, 343160, 343161, 343162, 343163) c6( ) c7(ck, bM, ou, x, fX, eE, cj, zV, k, ox, aVU, ajo, f, lM, iL, Us); #c1(CRLF3) c2(NP_057070) c3(cytokine receptor-like factor 3) c4(188768, 188769) c5(188770, 188771, 188772, 188773, 188774, 188775) c6( ) c7(aVV, T, d, id, kS, ds, nX, Az, CT, aq, ey, eA, jj, zy, t, xb, B, xt); #c1(CRLF3) c2(XP_011523181) c3 (cytokine receptor-like factor 3 isoform X2) c4( ) c5(317711, 317712, 317713, 317714, 317715, 317716) c6( ) c7(aVV, T, d, id, kS, ds, nX, Az, CT, aq, ey, eA, jj, zy, t, xb, B, xt); #c1(CRLS1) c2(NP_001120930) c3(cardiolipin synthase isoform 2) c4(44945) c5(44946, 44947, 44948, 44949, 44950, 44951) c6( ) c7(v, aVW, K, fD, fh, ao, alk); #c1(CRLS1) c2(NP_061968) c3(cardiolipin synthase isoform 1) c4(201348, 201349) c5(201350, 201351, 201352, 201353, 201354, 201355) c6( ) c7(v, aVW, K, fD, fh, ao, alk); #c1(CRLS1) c2(XP_011527565) c3(cardiolipin synthase isoform X3) c4( ) c5(322410, 322411, 322412, 322413, 322414, 322415) c6( ) c7(v, aVW, K, fD, fh, ao, alk); #c1(CRMP1) c2(NP_001014809) c3(dihydropyrimidinase-related protein 1 isoform 1) c4(26000) c5(26001, 26002, 26003, 26004, 26005, 26006) c6( ) c7(ID, T, aOH, dG, pd, Na, Ae, cy, Nc, K, an, ly, g, cm, bz, lx, ck, wg, c, eM, aND, U, iu); #c1(CRMP1) c2(NP_001275590) c3(dihydropyrimidinase-related protein 1 isoform 3) c4(106856) c5(106857, 106858, 106859, 106860, 106861, 106862) c6( ) c7(ID, T, aOH, dG, pd, Na, Ae, cy, Nc, K, an, ly, g, cm, bz, lx, ck, wg, c, eM, aND, U, iu); #c1(CRNKL1) c2(NP_001265554) c3(crooked neck-like protein 1 isoform b) c4(98828) c5(98829, 98830, 98831, 98832, 98833, 98834) c6( ) c7(fG, h, Nc, iV, hE, cd); #c1(CRNKL1) c2(NP_057736) c3(crooked neck-like protein 1 isoform a) c4(192586) c5(192587, 192588, 192589, 192590, 192591, 192592) c6( ) c7(fG, h, Nc, iV, hE, cd); #c1(CRNN) c2(NP_057274) c3(cornulin) c4(189794, 189795) c5(189796, 189797, 189798, 189799, 189800, 189801, 189802, 189803, 189804, 189805, 189806, 189807) c6( ) c7(pN, cE, ht, ff, K, en, hu, Xk, it, MG); #c1 (CROCC) c2(XP_011540774) c3(rootletin isoform X6) c4( ) c5(339353) c6( ) c7(B); #c1(CROT) c2(NP_001137407) c3(peroxisomal carnitine 0-octanoyltransferase isoform 1) c4(55635) c5(55636, 55637, 55638, 55639, 55640, 55641) c6( ) c7(Y); #c1(CROT) c2(NP_001230674) c3(peroxisomal carnitine 0-octanoyltransferase isoform 3) c4(86546) c5(86547, 86548, 86549, 86550, 86551, 86552) c6( ) c7(Y); #c1(CROT) c2(NP_066974) c3(peroxisomal carnitine 0-octanoyltransferase isoform 2) c4(207929) c5(207930, 207931, 207932, 207933, 207934, 207935) c6( ) c7(Y); #c1(CRP) c2(XP_011507509) c3(C-reactive protein isoform X1) c4(297404, 297405, 297406, 297407) c5(297408, 297409, 297410, 297411, 297412, 297413, 297414, 297415, 297416, 297417, 297418, 297419, 297420, 297421, 297422, 297423, 297424, 297425, 297426, 297427, 297428, 297429, 297430, 297431, 297432, 297433, 297434, 297435, 297436, 297437, 297438, 297439) c6( ) c7(oO, bM, T, fR, pf, ea, aUT, e, al, bO, aza, bB, eb, fd, gf, aY, fw, aMB, AW, dY, Bc, rw, M, dV, aWa, Bi, K, GO, hu, dQ, qM, aWf, eN, avn, DY, VF, aa, hm, aAu, aW, aEH, az, Rr, hl, ih, fc, vs, zC, NR, pl, eF, axp, akw, uC, Uk, bm, aWd, eY, y, lt, rq, eM, wE, WS, mx, U, tV, uo, Zn, vn, vy, mk, m, wx, eg, QF, P, nD, dD, sd, sw, tp, aw, ht, NT, ez, n, aj, fr, ct, JW, cd, ai, aJr, aDF, rc, eW, jz, Zu, Yc, ec, afn, gq, rC, cU, axE, jy, dL, h, eV, ux, uV, Um, aGL, wC, A, mR, sp, Xg, fQ, aq, fe, pm, cM, adz, hq, ar, kT, W, dC, fa, lf, mg, hL, RJ, E, ut, uz, aVY, fL, hR, at, Pr, B, cB, it, L, bL, jv, aAf, pf, eR, mr, atB, wL, uv, ex, cz, sM, cR, v, j, C, sl, lu, nS, q, J, ady, PW, cF, aWb, yw, aWc, rS, aT, agD, cy, dz, bK, bq, ae, af, eu, uA, tn, kl, bf, s, fO, lC, bg, r, lo, rs, en, cl, wA, t, iK, pb, lY, rR, wg, tW, ac, iM, aWe, hc, dh, fP, hO, wR, mf, dy, oX, fD, Kd, gV, Ve, am, Ke, aag, aZ, em, vr, aET, bS, dG, eq, wV, atK, mC, ad, dZ, hX, wD, fU, av, LL, od, db, sN, sO, oz, aCJ, ds, NS, oW, Os, qV, v,J, JK, lQ, uD, vl, na, sj, qE, aAy, aVZ, je, hG, cH, vj, ff, eH, gC, Ce, cr, kD, dq, eZ, uF, iV, pT, YZ, Z, bz, aVX, qa, ek, amS, pc, nb, atM, bE, ag, X, eB, gZ, MJ, aS, ei, Lz, GF, hE, xg, st, hv); #c1 (CRTAC1) c2(NP_001193457) c3(cartilage acidic protein 1 isoform B precursor) c4(82194, 82195, 82196) c5(82197, 82198, 82199, 82200, 82201, 82202, 82203, 82204, 82205, 82206, 82207, 82208, 82209, 82210, 82211, 82212) c6(82213, 82214, 82215, 82216) c7(bL, bM, tk, aHY); #c1(CRTAC1) c2(NP_060528) c3(cartilage acidic protein 1 isoform A precursor) c4(196402, 196403) c5(196404, 196405, 196406, 196407, 196408, 196409, 196410, 196411) c6(196412, 196413) c7(bL, bM, tk, aHY); #c1(CRTAC1) c2(XP_005269995) c3(cartilage acidic protein 1 isoform X2) c4( ) c5(283010, 283011, 283012, 283013, 283014, 283015, 283016, 283017, 283018, 283019, 283020, 283021, 283022, 283023, 283024, 283025) c6(283026, 283027, 283028, 283029) c7(bL, bM, tk, aHY); #c1(CRTAM) c2(NP_001291711) c3(cytotoxic and regulatory T-cell molecule Isoform 2 precursor) c4(116369) c5(116370, 116371, 116372, 116373, 116374, 116375) c6( ) c7(hS); #c1(CRTAM) c2(NP_062550) c3(cytotoxic and regulatory T-cell molecule Isoform 1 precursor) c4(201943) c5(201944, 201945, 201946, 201947, 201948, 201949) c6( ) c7(hS); #c1(CRTAP) c2(NP_006362) c3(cartilage-associated protein precursor) c4(164351, 164352, 164353, 164354) c5(164355, 164356, 164357, 164358, 164359, 164360, 164361, 164362, 164363, 164364, 164365, 164366, 164367, 164368, 164369, 164370, 164371, 164372, 164373, 164374, 164375, 164376, 164377, 164378) c6( ) c7(bA, T, zw, eM, ik, PV, uo, ci, zp, aWh, ade, fb, V, bz, aWg); #c1(CRTC1) c2(NP_001091952) c3(CREB-regulated transcription coactivator 1 isoform 3) c4(38996) c5(38997, 38998, 38999, 39000, 39001, 39002) c6( ) c7(T, hl, wi, aWj, pf, iB, fd, v, ad, afb, di, lg, aWi, aex, M, aT, cy, Tj, ahG, aW, Y, bz, es, iA, FB, dJ); #c1(CRTC1) c2(NP_056136) c3(CREB-regulated transcription coactivator 1 isoform 1) c4(185888) c5(185889, 185890, 185891, 185892, 185893, 185894) c6( ) c7(T, hl, wi, aWj, pf, iB, fd, v, ad, afb, di, lg, aWi, aex, M, aT, cy, Tj, ahG, aW, Y, bz, es, iA, FB, dJ); #c1(CRTC1) c2(XP_005259891) c3(CREB-regulated transcription coactivator 1 isoform X3) c4(276313) c5(276314, 276315, 276316, 276317, 276318, 276319) c6( ) c7(T, hl, wi, aWj, pf, iB, fd, v, ad, afb, di, lg, aWi, aex, M, aT, cy, Tj, ahG, aW, Y, bz, es, iA, FB, dJ); #c1 (CRTC2) c2(NP_859066) c3(CREB-regulated transcription coactivator 2) c4(255474) c5(255475, 255476, 255477, 255478, 255479, 255480) c6( ) c7(bL, T, d, aW, ff, aT, fd, aj, mf, dG, K, v, bq, en, g, bz, J, ad); #c1(CRTC3) c2(NP_001036039) c3(CREB-regulated transcription coactivator 3 isoform b) c4(33794, 33795) c5(33796, 33797, 33798, 33799, 33800, 33801, 33802, 33803, 33804, 33805, 33806, 33807) c6( ) c7(v, iB, aaw); #c1(CRTC3) c2(NP_073606) c3(CREB-regulated transcription coactivator 3 isoform a) c4(213051) c5(213052, 213053, 213054, 213055, 213056, 213057) c6( ) c7(v, iB, aaw); #c1(CRX) c2(NP_000545) c3(cone-rod homeobox protein) c4(12596) c5(12597, 12598, 12599, 12600, 12601, 12602, 12603, 12604, 12605, 12606, 12607, 12608, 12609, 12610, 12611, 12612, 12613, 12614, 12615, 12616, 12617, 12618, 12619, 12620, 12621, 12622) c6( ) c7(aWk, T, nx, ahX, nA, Nh, Sr, MU, lR, aNM, ru, fe, Ud, nC, ayj, aWl, W, bP, nw, eQ, OL, de); #c1(CRY1) c2(NP_004066) c3(cryptochrome-1) c4(143262) c5(143263, 143264, 143265, 143266, 143267, 143268, 143269, 143270) c6(143271, 143272) c7(T, bO, cz, aj, bS, ct, q, J, ad, aT, bk, ae, jJ, hZ, sj, aWm, iU, aW, Y, r, cl, Z, hq, aPY, cj); #c1(CRY1) c2(XP_011536241) c3(cryptochrome-1 isoform X1) c4(333580) c5(333581, 333582, 333583, 333584, 333585, 333586) c6( ) c7(T, bO, cz, aj, bS, ct, q, J, ad, aT, bK, ae, jJ, hZ, sj, aWm, iU, aW, Y, r, cl, Z, hq, aPY, cj); #c1(CRY2) c2(NP_001120929) c3(cryptochrome-2 isoform 2) c4(44938) c5(44939, 44940, 44941, 44942, 44943, 44944) c6( ) c7(bL, aMK, T, iW, jx, jh, cz, bS, v, ct, ci, ad, aT, ta, bK, ae, iU, aW, g, sz, Z, hq, aPY, amt, qJ, nM, cA, ei); #c1(CRY2) c2(NP_066940) c3(cryptochrome-2 isoform 1) c4(207616) c5(207617, 207618, 207619, 207620, 207621, 207622) c6( ) c7(bL, aMK, T, iW, jx, jh, cz, bS, v, ct, ci, ad, aT, ta, bk, ae, iU, aW, g, sz, Z, hq, aPY, amt, qJ, nM, cA, ei); #c1(CRYAB) c2(NP_001276737) c3(alpha-crystallin B chain) c4(108290, 108291, 108292, 108293, 108294) c5(108295, 108296, 108297, 108298, 108299, 108300, 108301, 108302, 108303, 108304, 108305, 108306, 108307, 108308, 108309, 108310, 108311, 108312, 108313, 108314, 108315, 108316, 108317, 108318, 108319, 108320, 108321, 108322, 108323, 108324, 108325, 108326, 108327, 108328, 108329, 108330, 108331, 108332, 108333, 108334, 108335, 108336, 108337, 108338, 108339, 108340, 108341, 108342, 108343, 108344, 108345, 108346, 108347, 108348, 108349) c6( ) c7(T, dt, aWt, sw, fG, ht, qy, GC, n, arl, j, ci, asF, PJ, mF, fr, V, aWr, eA, ad, ku, b, cc, Ws, aWq, akJ, oj, aT, vc, aWs, K, db, af, JU, d, aW, ff, Ej, aWo, en, aWp, t, iK, LB, kP, jV, W, bP, X, mx, aWn, U, nw, eo, m, i, rA, B); #c1(CRYBA1) c2(NP_005199) c3(beta-crystallin A3) c4(153837) c5(153838, 153839, 153840, 153841, 153842, 153843) c6( ) c7(j, Ew, aWu, wf); #c1(CRYBA2) c2(NP_476434) c3(beta-crystallin A2) c4(230576, 230577) c5(230578, 230579, 230580, 230581, 230582, 230583, 230584, 230585, 230586, 230587, 230588, 230589, 230590, 230591, 230592, 230593) c6( ) c7(Km); #c1(CRYBA4) c2(NP_001877) c3(beta-crystallin A4) c4( ) c5(121927, 121928, 121929, 121930, 121931) c6( ) c7(arm, aWw, kj, aWv); #c1(CRYBA4) c2(XP_011528200) c3(beta-crystallin A4 isoform X1) c4(323290) c5(323291) c6( ) c7(arm, aWw, kj, aWv); #c1(CRYBB1) c2(NP_001878) c3(beta-crystallin B1) c4(121932) c5(121933, 121934, 121935, 121936, 121937, 121938) c6( ) c7(aWx, arm, aWy); #c1(CRYBB2) c2(NP_000487) c3(beta-crystallin B2) c4(9829) c5(9830, 9831, 9832, 9833, 9834, 9835) c6( ) c7(kj, aWA, arm, Or, Zh, aWz, qW); #c1(CRYBB3) c2(NP_004067) c3(beta-crystallin B3) c4( ) c5(143273, 143274, 143275, 143276, 143277) c6( ) c7(aWB); #c1(CRYBB3) c2(XP_011528204) c3(beta-crystallin B3 isoform X1) c4(323292) c5(323293) c6( ) c7(aWB); #c1(CRYGB) c2(NP_005201) c3(gamma-crystallin B) c4(153844) c5(153845, 153846, 153847, 153848, 153849, 153850) c6( ) c7(aWC); #c1(CRYGC) c2(NP_066269) c3(gamma-crystallin C) c4(206867) c5(206868, 206869, 206870, 206871, 206872, 206873) c6( ) c7(bM, zT, apn, eZ, cm, h, nw, g, aWz, V); #c1(CRYGD) c2(NP_008822) c3(gamma-crystallin D) c4(168825) c5(168826, 168827, 168828, 168829, 168830, 168831) c6( ) c7(aWD, T, dD, ea, e, aWG, KO, cl, eq, apt, uE, q, Uy, o, ad, aoH, ai, yM, wD, rw, yu, ae, A, aWE, bg, r, pL, bz, ba, y, ac, zr, aWF, QF, uz, bC, anx, aWz, iu, VB, XL); #c1(CRYGS) c2(NP_060011) c3(beta-crystallin S) c4(193634) c5(193635, 193636, 193637, 193638, 193639, 193640, 193641, 193642, 193643, 193644) c6( ) c7(aWH, arm, K, Zh, aWI); #c1(CRYL1) c2(NP_057058) c3(lambda-crystallin homolog) c4(188680) c5(188681, 188682, 188683, 188684, 188685, 188686) c6( ) c7(dc, bo, aWJ, wz, jy, pw, B, ad); #c1(CRYM) c2(NP_001879) c3 (ketimine reductase mu-crystallin) c4(121939, 121940) c5(121941, 121942, 121943, 121944, 121945, 121946, 121947, 121948, 121949, 121950, 121951, 121952, 121953, 121954, 121955, 121956, 121957, 121958, 121959) c6( ) c7(T, rx); #c1(CRYZL1) c2(NP_665857) c3(quinone oxidoreductase-like protein 1) c4(24/762, 24/763, 24/764, 24/765) c5(24/766, 241767, 24/768, 24/769, 24/770, 241771, 241772, 241773, 241774, 241775, 241776, 24/777, 24/778, 241779, 241780, 241781, 24/782, 241783, 241784, 241785, 241786, 241787, 241788, 24/789) c6( ) c7(bS); #c1(CS) c2(NP_004068) c3(citrate synthase, mitochondrial precursor) c4(143278, 143279) c5(143280, 143281, 143282, 143283, 143284, 143285, 143286, 143287, 143288, 143289, 143290, 143291) c6( ) c7(T, aWK, aC, ac, c, cc, aWL, Ue, aWM, mx, dV, cd, aNG, LE, v, as, aMB, cm, aoz, bN); #c1 (CSAD) c2(NP_001231634) c3(cysteine sulfinic acid decarboxylase isoform 2) c4( ) c5(87366, 87367, 87368, 87369, 87370, 87371, 87372, 87373, 87374, 87375) c6( ) c7(Y, fG, fQ, uu, ur, la, lR, ae, pb, ad); #c1(CSAD) c2(NP_057073) c3(cysteine sulfinic acid decarboxylase isoform 1) c4(188785, 188786, 188787, 188788, 188789, 188790) c5(188791, 188792, 188793, 188794, 188795, 188796, 188797, 188798, 188799, 188800, 188801, 188802, 188803, 188804, 188805) c6( ) c7(Y, fG, fQ, uu, ur, la, lR, ae, pb, ad); #c1(CSAD) c2(XP_011536744) c3(cysteine sulfinic acid decarboxylase isoform X2) c4(334413) c5(334414, 334415, 334416, 334417, 334418) c6( ) c7(Y, fG, fQ, uu, ur, la, lR, ae, pb, ad); #c1(CSAD) c2(XP_011536746) c3(cysteine sulfinic acid decarboxylase isoform X3) c4( ) c5(334419) c6( ) c7(Y, fG, fQ, uu, ur, la, lR, ae, pb, ad); #c1(CSAD) c2(XP_011536749) c3(cysteine sulfinic acid decarboxylase isoform X5) c4( ) c5(334420, 334421, 334422, 334423, 334424) c6( ) c7(Y, fG, fQ, uu, ur, la, lR, ae, pb, ad); #c1(CSAD) c2(XP_011536750) c3(cysteine sulfinic acid decarboxylase isoform X6) c4( ) c5(334425, 334426, 334427, 334428, 334429, 334430, 334431, 334432, 334433, 334434) c6( ) c7(Y, fG, fQ, uu, ur, la, lR, ae, pb, ad); #c1(CSAG3) c2(NP_001123300) c3(chondrosarcoma-associated gene 2/3 protein isoform b) c4(46877) c5(46878, 46879, 46880, 46881, 46882, 46883) c6( ) c7(T, id, eH, aT, m, bu, eF); #c1 (CSEIL) c2(NP_001243064) c3(exportin-2 isoform 2) c4(89708, 89709) c5(89710, 89711, 89712, 89713, 89714, 89715, 89716, 89717, 89718, 89719, 89720, 89721) c6( ) c7(T, fM, bB, fr, ci, Fc, q, eA, anU, aT, h, dx, d, aiB, s, aW, vj, ev, ff, r, ks, aq, en, anW, iR, t, HI, ck, bP, bE, anY, iS, np, m, y, B); #c1(CSEIL) c2(NP_001307) c3 (exportin-2 isoform 1) c4(116563, 116564) c5(116565, 116566, 116567, 116568, 116569, 116570, 116571, 116572, 116573, 116574, 116575, 116576) c6( ) c7(T, fM, bB, fr, ci, Fc, q, eA, anU, aT, h, dx, d, aiB, s, aW, vj, ev, ff, r, ks, aq, en, anW, iR, t, HI, ck, bP, bE, anY, iS, np, m, y, B); #c1(CSFIR) c2(NP_005202) c3(macrophage colony-stimulating factor 1 receptor precursor) c4(153851, 153852, 153853, 153854) c5(153855, 153856, 153857, 153858, 153859, 153860, 153861, 153862, 153863, 153864, 153865, 153866, 153867, 153868, 153869, 153870, 153871, 153872, 153873, 153874, 153875, 153876, 153877, 153878) c6( ) c7(bM, T, aC, Js, ea, bB, ci, x, DY, eG, aWN, aW, hl, g, eF, bw, iT, nB, AM, k, m, dD, n, fr, br, ft, RN, By, dL, ey, dx, Gq, aWO, kz, Ei, hL, at, aWP, eo, bD, B, Jo, afK, V, eA, aT, j, aPF, an, ae, aeY, bA, hf, cl, t, ck, ahN, nM, ac, aWQ, dT, bt, ao, oA, bs, jh, dG, eq, hF, ad, dP, hd, py, iY, hP, ji, cb, nJ, TC, d, eH, dq, gE, aWR, es, EW, zr, bE, ME, y, jr); #c1(CSF2) c2(NP_000749) c3(granulocyte-macrophage colony-stimulating factor precursor) c4(15157) c5(15158, 15159, 15160, 15161, 15162, 15163, 15164, 15165, 15166, 15167, 15168, 15169, 15170, 15171, 15172, 15173, 15174, 15175, 15176, 15177, 15178, 15179, 15180, 15181, 15182, 15183, 15184, 15185, 15186, 15187, 15188, 15189, 15190, 15191, 15192, 15193, 15194, 15195, 15196, 15197, 15198, 15199, 15200, 15201, 15202, 15203, 15204, 15205, 15206, 15207, 15208, 15209, 15210, 15211, 15212, 15213, 15214, 15215, 15216, 15217, 15218, 15219, 15220, 15221, 15222, 15223, 15224, 15225, 15226, 15227, 15228, 15229, 15230, 15231, 15232, 15233, 15234, 15235, 15236, 15237, 15238, 15239, 15240, 15241, 15242, 15243, 15244, 15245, 15246, 15247, 15248, 15249, 15250, 15251, 15252, 15253, 15254, 15255, 15256, 15257, 15258, 15259, 15260, 15261, 15262, 15263, 15264, 15265, 15266, 15267, 15268, 15269, 15270, 15271, 15272, 15273, 15274, 15275, 15276, 15277, 15278, 15279, 15280, 15281, 15282, 15283, 15284, 15285, 15286, 15287, 15288, 15289, 15290, 15291, 15292, 15293, 15294, 15295, 15296, 15297, 15298, 15299, 15300, 15301, 15302, 15303) c6(15304, 15305, 15306) c7(T, os, uk, ea, fd, ci, aE, aXn, Ds, aXg, aXa, aiJ, cp, aa, dg, akw, jV, iA, cj, Zn, k, act, aWX, aEw, aFG, aXw, aj, qW, cd, br, ox, nP, sp, fe, CA, pm, ajz, adz, RB, aWV, dB, abW, jv, cz, bX, aXr, q, Cc, rO, jj, rz, eu, bA, lo, cl, pb, aXi, Cv, cT, iM, ac, ig, mG, aXt, bt, Ze, rA, gV, iW, jN, jh, oZ, dG, Gn, fu, aNH, ad, hX, iC, vJ, akJ, oW, iY, cb, qZ, ff, dq, bz, ok, akC, lC, bM, bB, asr, ip, x, OG, eG, aiV, Oj, kt, as, eE, eF, cm, bw, bb, anT, vA, oy, aiM, hh, xN, aiu, dD, Ht, n, oY, Mo, Hx, no, cq, Bj, DU, aWY, pi, cC, A, fJ, lb, hQ, po, bP, aXk, at, pg, bk, eo, B, ek, qc, ul, dA, ti, afK, aWU, Do, V, eJ, Yn, WY, aql, aWW, ae, lr, jf, tn, iZ, De, WQ, bu, t, UZ, rY, ck, ll, HK, JH, oX, hl, pY, df, eq, CJ, db, JK, rL, cs, d, je, hG, xH, Z, bF, dO, Hs, y, aXu, kS, CW, pC, JN, Js, zs, l, axk, qj, lR, Ap, wa, Qk, rB, K, eL, apn, g, uC, iP, aJX, aWZ, AM, U, PV, dW, m, XL, Ol, aw, aBl, eh, ft, cU, Ha, alY, jT, cM, fs, bo, mg, JM, aXv, aEG, Pr, Jo, L, fM, fG, Nt, ON, j, pd, eA, J, XX, hb, jZ, apJ, aeY, sb, aCr, Y, aM, RF, Pg, r, en, wA, aoG, iK, bN, Qi, tj, Dy, c, auo, aXe, hA, Uq, rK, dy, fD, LS, sY, id, bs, bS, EA, hF, ase, vN, dP, vB, ji, lR, eH, cr, BG, iR, Nq, TA, Bv, aHL, asq, aXq, fV, aXb, mF, aR, lE, Fo, aU, bU, H, aXj, aXp, amN, DA, aW, aXc, aXh, eM, iT, gn, R, qR, QS, aFc, aXo, fr, ct, aWT, ai, eW, aXl, ke, h, SH, Nf, dL, aXd, Nr, aXf, aq, op, Qu, ahC, bL, Dw, eR, C, ze, cF, aaF, aT, dz, adV, an, af, ak, s, iU, oV, aaE, xw, dS, aXm, agc, yW, vZ, AH, dT, ij, aWS, hS, qX, aD, Fb, oH, jE, av, F, sO, abg, ul, TC, yx, aGS, eZ, aXs, ek, es, pc, zr, aqM, bE, z, dJ, fh, X, Ex); #c1(CSF2RA) c2(NP_001155002) c3(granulocyte-macrophage colony-stimulating factor receptor subunit alpha isoform f precursor) c4(61261) c5(61262, 61263, 61264, 61265, 61266, 61267) c6( ) c7(aWW, akt, ase, vJ, dT); #c1(CSF2RA) c2(NP_001155004) c3(granulocyte-macrophage colony-stimulating factor receptor subunit alpha isoform h) c4( ) c5(61268, 61269, 61270, 61271, 61272) c6( ) c7(aWW, akt, ase, vJ, dT); #c1(CSF2RA) c2(NP_758449) c3(granulocyte-macrophage colony-stimulating factor receptor subunit alpha isoform b precursor) c4(249188) c5(249189, 249190, 249191, 249192, 249193, 249194) c6( ) c7(aWW, akt, ase, vJ, dT); #c1(CSF2RA) c2(NP_758452) c3(granulocyte-macrophage colony-stimulating factor receptor subunit alpha isoform e precursor) c4(249195) c5(249196, 249197, 249198, 249199, 249200, 249201, 249202, 249203, 249204, 249205, 249206, 249207) c6( ) c7(aWW, akt, ase, vJ, dT); #c1(CSF2RA) c2(XP_011543921) c3(granulocyte-macrophage colony-stimulating factor receptor subunit alpha isoform X1) c4(343108, 343109, 343110, 343111) c5(343112, 343113, 343114, 343115, 343116, 343117, 343118, 343119, 343120, 343121, 343122, 343123, 343124, 343125, 343126) c6( ) c7(aWW, akt, ase, vJ, dT); #c1(CSF2RA) c2(XP_011543925) c3(granulocyte-macrophage colony-stimulating factor receptor subunit alpha isoform X5) c4(343127) c5( ) c6( ) c7(aWW, akt, ase, vJ, dT); #c1(CSF2RA) c2(XP_011543927) c3(granulocyte-macrophage colony-stimulating factor receptor subunit alpha isoform X6) c4(343128) c5(343129, 343130, 343131, 343132, 343133, 343134) c6( ) c7(aWW, akt, ase, vJ, dT); #c1(CSF2RA) c2(XP_011543930) c3(granulocyte-macrophage colony-stimulating factor receptor subunit alpha isoform X7) c4(343135) c5(343136, 343137, 343138, 343139, 343140, 343141, 343142, 343143, 343144, 343145, 343146, 343147, 343148, 343149, 343150, 343151, 343152, 343153, 343154, 343155, 343156, 343157) c6( ) c7(aWW, akt, ase, vJ, dT); #c1(CSF2RB) c2(NP_000386) c3(cytokine receptor common subunit beta precursor) c4(8371, 8372) c5(8373, 8374, 8375, 8376, 8377, 8378, 8379, 8380, 8381, 8382, 8383, 8384) c6( ) c7(ck, bM, mR, aWV, ayU, bO, dT, aXx, he, bt, eu); #c1(CSF2RB) c2(XP_005261397) c3(cytokine receptor common subunit beta isoform X3) c4(277346, 277347) c5(277348, 277349, 277350, 277351, 277352, 277353, 277354, 277355, 277356, 277357, 277358, 277359) c6( ) c7(ck, bM, mR, aWV, ayU, bO, dT, aXx, he, bt, eu); #c1(CSF2RB) c2(XP_011528206) c3(cytokine receptor common subunit beta isoform X2) c4(323294) c5( ) c6( ) c7(ck, bM, mR, aWV, ayU, bO, dT, aXx, he, bt, eu); #c1(CSF2RB) c2(XP_011528207) c3(cytokine receptor common subunit beta isoform X4) c4( ) c5(323295, 323296, 323297, 323298, 323299, 323300) c6( ) c7(ck, bM, mR, aWV, ayU, bO, dT, aXx, he, bt, eu); #c1(CSF3) c2(NP_000750) c3(granulocyte colony-stimulating factor isoform a precursor) c4(15307) c5(15308, 15309, 15310, 15311, 15312, 15313, 15314, 15315, 15316, 15317, 15318) c6(15319, 15320) c7(oO, bM, T, os, xS, fR, fV, qx, al, fd, bB, ci, amW, aR, ip, x, aE, aEX, fm, DO, Ds, K, ex, eL, Us, aa, aW, hC, hl, bp, as, eF, cm, uC, bw, eM, iT, aWZ, mx, tV, cj, dW, k, m, gn, dD, aw, ht, n, oY, ct, aXA, cd, br, ai, ke, ft, cc, hw, azD, qg, aXE, h, eV, hn, dL, DU, pj, dx, afu, Nr, aXy, XE, aXG, fQ, aq, po, fe, pm, kT, hk, kz, aXF, Vy, lf, at, pg, aEG, Pr, B, Dw, fM, dA, fG, jx, eX, aA, afK, hy, j, v, C, bX, aoC, Do, eA, er, zn, aXC, hW, dn, aT, cy, dz, jj, ae, bA, sb, lE, iU, oV, Y, en, t, ik, aXi, ck, BU, wg, ac, c, hA, il, QG, aXB, CZ, rk, dy, EY, oX, ud, bt, hS, kV, Kd, sY, gV, id, qb, iW, df, ol, jh, bs, oZ, eq, aXD, hF, atK, ad, hX, av, oz, oj, oW, ji, nJ, aXz, d, BN, IR, hG, et, ff, eH, cr, dN, aoF, af, aot, aGj, bz, dr, ok, Nq, bE, eB, ll, gZ, aS, hE, kS, X); #c1(CSF3) c2(NP_001171618) c3(granulocyte colony-stimulating factor isoform d precursor) c4( ) c5(70844, 70845, 70846, 70847, 70848, 70849, 70850, 70851, 70852, 70853, 70854, 70855, 70856, 70857, 70858, 70859, 70860, 70861, 70862, 70863, 70864, 70865, 70866, 70867, 70868, 70869, 70870, 70871, 70872, 70873, 70874, 70875, 70876, 70877, 70878, 70879, 70880, 70881, 70882, 70883, 70884, 70885, 70886, 70887, 70888, 70889, 70890, 70891, 70892, 70893, 70894, 70895, 70896, 70897, 70898, 70899, 70900, 70901, 70902, 70903, 70904, 70905, 70906, 70907, 70908, 70909, 70910, 70911, 70912, 70913, 70914, 70915, 70916, 70917, 70918, 70919, 70920, 70921, 70922, 70923, 70924, 70925, 70926, 70927, 70928, 70929, 70930, 70931, 70932) c6(70933) c7(oO, bM, T, os, xS, fR, fV, qx, al, fd, bB, ci, amW, aR, ip, x, aE, aEX, fm, DO, Ds, K, ex, eL, Us, aa, aW, hC, hl, bp, as, eF, cm, uC, bw, eM, iT, aWZ, mx, tV, cj, dW, k, m, gn, dD, aw, ht, n, oY, ct, aXA, cd, br, ai, ke, ft, cc, hw, azD, qg, aXE, h, eV, hn, dL, DU, pj, dx, afu, Nr, aXy, XE, aXG, fQ, aq, po, fe, pm, kT, hk, kz, aXF, Vy, lf, at, pg, aEG, Pr, B, Dw, fM, dA, fG, jx, eX, aA, afK, hy, j, v, C, bX, aoC, Do, eA, er, zn, aXC, hW, dn, aT, cy, dz, jj, ae, bA, sb, lE, iU, oV, Y, en, t, iK, aXi, ck, BU, wg, ac, c, hA, iQ, QG, aXB, CZ, rk, dy, EY, oX, ud, bt, hS, kV, Kd, sY, gV, id, qb, iW, df, ol, jh, bs, oZ, eq, aXD, hF, atK, ad, hX, av, oz, oj, oW, ji, nJ, aXz, d, BN, lR, hG, et, ff, eH, cr, dN, aoF, aF, aot, aGj, bz, dr, ok, Nq, bE, eB, ll, gZ, aS, hE, KS, X); #c1(CSF3) c2(NP_757373) c3(granulocyte colony-stimulating factor isoform b precursor) c4(248747, 248748, 248749, 248750, 248751, 248752, 248753) c5(248754, 248755, 248756, 248757, 248758, 248759, 248760, 248761, 248762, 248763, 248764, 248765, 248766, 248767, 248768, 248769, 248770, 248771, 248772, 248773, 248774, 248775, 248776, 248777, 248778, 248779, 248780, 248781, 248782, 248783, 248784, 248785, 248786, 248787, 248788, 248789, 248790, 248791, 248792, 248793, 248794, 248795, 248796, 248797, 248798, 248799, 248800, 248801, 248802, 248803, 248804, 248805, 248806, 248807, 248808, 248809, 248810, 248811, 248812, 248813, 248814, 248815, 248816, 248817, 248818, 248819, 248820, 248821, 248822, 248823, 248824, 248825, 248826, 248827, 248828, 248829, 248830, 248831, 248832, 248833, 248834, 248835, 248836, 248837, 248838, 248839, 248840, 248841, 248842, 248843, 248844, 248845, 248846, 248847, 248848, 248849, 248850, 248851, 248852, 248853, 248854, 248855, 248856, 248857, 248858, 248859, 248860, 248861, 248862, 248863, 248864, 248865, 248866, 248867, 248868, 248869, 248870, 248871, 248872, 248873, 248874, 248875, 248876, 248877, 248878, 248879, 248880, 248881, 248882, 248883, 248884, 248885, 248886, 248887, 248888, 248889, 248890, 248891, 248892, 248893, 248894, 248895, 248896, 248897, 248898, 248899, 248900, 248901, 248902, 248903, 248904, 248905, 248906, 248907, 248908, 248909, 248910, 248911, 248912, 248913, 248914, 248915, 248916, 248917, 248918, 248919, 248920, 248921, 248922, 248923, 248924, 248925, 248926, 248927, 248928, 248929, 248930, 248931, 248932, 248933, 248934, 248935, 248936, 248937, 248938, 248939, 248940, 248941, 248942, 248943, 248944, 248945, 248946, 248947, 248948, 248949, 248950, 248951, 248952, 248953, 248954, 248955, 248956, 248957, 248958, 248959, 248960, 248961, 248962, 248963, 248964, 248965, 248966, 248967, 248968, 248969, 248970, 248971, 248972, 248973, 248974, 248975, 248976, 248977, 248978, 248979, 248980, 248981, 248982, 248983, 248984, 248985, 248986, 248987, 248988, 248989, 248990, 248991, 248992, 248993, 248994, 248995, 248996, 248997, 248998, 248999, 249000, 249001, 249002, 249003, 249004, 249005, 249006, 249007, 249008, 249009, 249010, 249011, 249012, 249013, 249014, 249015, 249016, 249017, 249018, 249019, 249020, 249021, 249022, 249023, 249024, 249025, 249026, 249027, 249028, 249029, 249030) c6(249031, 249032, 249033, 249034, 249035, 249036, 249037, 249038, 249039, 249040, 249041, 249042, 249043, 249044, 249045, 249046, 249047, 249048, 249049, 249050, 249051, 249052, 249053, 249054, 249055, 249056, 249057, 249058, 249059, 249060, 249061, 249062, 249063, 249064, 249065, 249066, 249067, 249068, 249069, 249070, 249071, 249072, 249073) c7(oO, bM, T, os, xS, fR, fV, qx, al, fd, bB, ci, amW, aR, ip, x, aE, aEX, fm, DO, Ds, K, ex, eL, Us, aa, aW, hC, hl, bp, as, eF, cm, uC, bw, eM, iT, aWZ, mx, tV, cj, dW, k, m, gn, dD, aw, ht, n, oY, ct, aXA, cd, br, ai, ke, ft, cc, hw, azD, qg, aXE, h, eV, hn, dL, DU, pj, dx, afu, Nr, aXy, XE, aXG, fQ, aq, po, fe, pm, KT, hk, kz, aXF, Vy, lf, at, pg, aEG, Pr, B, Dw, fM, dA, fG, jx, eX, aA, afK, hy, j, v, C, bX, aoC, Do, eA, er, zn, aXC, hW, dn, aT, cy, dz, jj, ae, bA, sb, lE, iU, oV, Y, en, t, iK, aXi, ck, BU, wg, ac, c, hA, iQ, QG, aXB, CZ, rk, dy, EY, oX, ud, bt, hS, kV, Kd, sY, gV, id, qb, iW, df, ol, jh, bs, oZ, eq, aXD, hF, atK, ad, hX, av, oz, oj, oW, ji, nJ, aXz, d, BN, lR, hG, et, ff, eH, cr, dN, aof, aF, aot, aGj, bz, dr, ok, Nq, bE, eB, ll, gZ, aS, hE, KS, X); #c1(CSF3) c2(NP_757374) c3(granulocyte colony-stimulating factor isoform c precursor) c4(249074, 249075, 249076) c5(249077, 249078, 249079, 249080, 249081, 249082, 249083, 249084, 249085) c6(249086, 249087) c7(oO, bM, T, os, xS, fR, fV, qx, al, fd, bB, ci, amW, aR, ip, x, aE, aEX, fm, DO, Ds, K, ex, eL, Us, aa, aW, hC, hl, bp, as, eF, cm, uC, bw, eM, iT, aWZ, mx, tV, cj, dW, k, m, gn, dD, aw, ht, n, oY, ct, aXA, cd, br, ai, ke, ft, cc, hw, azD, qg, aXE, h, eV, hn, dL, DU, pj, dx, afu, Nr, aXy, XE, aXG, fQ, aq, po, fe, pm, kT, hk, kz, aXF, Vy, lf, at, pg, aEG, Pr, B, Dw, fM, dA, fG, jx, eX, aA, afK, hy, j, v, C, bX, aoC, Do, eA, er, zn, aXC, hW, dn, aT, cy, dz, jj, ae, bA, sb, lE, iU, oV, Y, en, t, iK, aXi, ck, BU, wg, ac, c, hA, iQ, QG, aXB, CZ, rk, dy, EY, oX, ud, bt, hS, KV, Kd, sY, gV, id, qb, iW, df, ol, jh, bs, oZ, eq, aXD, hF, atK, ad, hX, av, oz, oj, oW, ji, nJ, aXz, d, BN, lR, hG, et, ff, eH, cr, dN, aoF, aF, aot, aGj, bz, dr, ok, Nq, bE, eB, ll, gZ, aS, hE, KS, X); #c1(CSGALNACT1) c2(NP_001123990) c3(chondroitin sulfate N-acetylgalactosaminyltransferase 1) c4(48171, 48172, 48173, 48174, 48175) c5(48176, 48177, 48178, 48179, 48180, 48181, 48182, 48183, 48184, 48185, 48186, 48187, 48188, 48189, 48190, 48191, 48192, 48193, 48194, 48195, 48196, 48197, 48198, 48199, 48200, 48201, 48202, 48203, 48204, 48205, 48206, 48207, 48208, 48209, 48210, 48211, 48212, 48213, 48214, 48215, 48216, 48217, 48218, 48219, 48220) c6( ) c7(DY, ql, oe, k, fu); #c1(CSGALNACT2) c2(NP_061060) c3(chondroitin sulfate N-acetylgalactosaminyltransferase 2) c4(199123) c5(199124, 199125, 199126, 199127, 199128, 199129) c6( ) c7(ahl); #c1(CSGALNACT2) c2(XP_011538240) c3(chondroitin sulfate N-acetylgalactosaminyltransferase 2 isoform X1) c4(335842) c5(335843, 335844, 335845, 335846, 335847, 335848) c6( ) c7(ahl); #c1(CSH1) c2(NP_001308) c3(chorionic somatomammotropin hormone 1 precursor) c4(116577, 116578) c5(116579, 116580, 116581, 116582, 116583, 116584, 116585, 116586, 116587, 116588, 116589, 116590, 116591, 116592, 116593, 116594, 116595, 116596, 116597, 116598, 116599, 116600, 116601, 116602, 116603) c6( ) c7(T, nl, wu, yk, Jy, ci, eW, aR, MR, aXH, jZ, nl, dV, aT, dz, KP, K, aAG, wm, nJ, vT, aW, LO, CT, nh, La, ik, bz, dr, ck, aXl, mg, U, hR, m, ei); #c1(CSH2) c2(NP_066271) c3(chorionic somatomammotropin hormone 2 isoform 1 precursor) c4(206874, 206875) c5(206876, 206877, 206878, 206879, 206880, 206881, 206882, 206883) c6( ) c7(T, nl, wu, yk, Jy, ci, eW, aR, MR, aXH, jZ, nl, dV, aT, dz, KP, K, aAG, wm, nJ, vT, aW, LO, CT, nh, La, ik, bz, dr, ck, aXl, mg, U, hR, m, ei); #c1(CSH2) c2(NP_072170) c3(chorionic somatomammotropin hormone 2 isoform 2 precursor) c4(212732, 212733) c5(212734, 212735, 212736, 212737, 212738, 212739, 212740, 212741, 212742, 212743, 212744, 212745, 212746, 212747) c6( ) c7(T, nl, wu, yk, Jy, ci, eW, aR, MR, aXH, jZ, nl, dV, aT, dz, KP, K, aAG, wm, nJ, vT, aW, LO, CT, nh, La, ik, bz, dr, ck, aXl, mg, U, hR, m, ei); #c1(CSH2) c2(NP_072171) c3(chorionic somatomammotropin hormone 2 isoform 3 precursor) c4(212748, 212749, 212750) c5(212751, 212752, 212753, 212754, 212755, 212756, 212757, 212758, 212759, 212760, 212761, 212762, 212763, 212764, 212765, 212766, 212767, 212768, 212769, 212770, 212771, 212772, 212773, 212774) c6(212775, 212776) c7(T, nl, wu, yk, Jy, ci, eW, aR, MR, aXH, jZ, nl, dV, aT, dz, KP, K, aAG, wm, nJ, vT, aW, LO, CT, nh, La, ik, bz, dr, ck, aXl, mg, U, hR, m, ei); #c1(CSHL1) c2(NP_001309) c3(chorionic somatomammotropin hormone-like 1 isoform 3) c4( ) c5(116604, 116605, 116606, 116607, 116608) c6( ) c7(bL, ck, T, A, d, aW, aT, bj, mf, aXJ, bq, zP, ae, ad, yv); #c1(CSHL1) c2(NP_072101) c3(chorionic somatomammotropin hormone-like 1 isoform 1 precursor) c4(212710, 212711) c5(212712, 212713, 212714, 212715, 212716, 212717, 212718, 212719, 212720, 212721, 212722, 212723) c6( ) c7(bL, ck, T, A, d, aW, aT, bj, mf, aXJ, bq, zP, ae, ad, yv): #c1(CSHL1) c2(NP_072102) c3(chorionic somatomammotropin hormone-like 1 isoform 4) c4(212724) c5(212725, 212726, 212727, 212728, 212729, 212730) c6( ) c7(bL, ck, T, A, d, aW, aT, bj, mf, axJ, bq, zP, ae, ad, yv); #c1(CSHL1) c2(NP_072103) c3(chorionic somatomammotropin hormone-like 1 isoform 2 precursor) c4(212731) c5( ) c6( ) c7(bL, ck, T, A, d, aW, aT, bj, mf, aXJ, bq, zP, ae, ad, yv); #c1(CSHL1) c2(XP_011522646) c3(chorionic somatomammotropin hormone-like 1 isoform X1) c4(317204) c5(317205, 317206, 317207, 317208, 317209, 317210, 317211, 317212, 317213) c6( ) c7(bL, ck, T, A, d, aW, aT, bj, mf, aXJ, bq, zP, ae, ad, yv); #c1(CSHL1) c2(XP_011522648) c3(chorionic somatomammotropin hormone-like 1 isoform X3) c4( ) c5(317214) c6( ) c7(bL, ck, T, A, d, aW, aT, bj, mf, aXJ, bq, zP, ae, ad, yv); #c1(CSK) c2(XP_005254222) c3(tyrosine-protein kinase CSK isoform X1) c4(273099, 273100) c5(273101, 273102, 273103, 273104, 273105, 273106, 273107, 273108, 273109, 273110, 273111, 273112, 273113, 273114, 273115, 273116, 273117, 273118, 273119, 273120) c6( ) c7(T, oe, lH, jx, aA, cz, bB, iJ, q, ad, aXK, yg, aT, h, K, ae, af, B, aW, Y, fO, eZ, r, aF, t, bw, pb, eM, bE, m, y, Pr, X, ao); #c1(CSMD1) c2(NP_150094) c3(CUB and sushi domain-containing protein 1 precursor) c4(227753, 227754, 227755, 227756, 227757, 227758) c5(227759, 227760, 227761, 227762, 227763, 227764, 227765, 227766, 227767, 227768, 227769, 227770, 227771, 227772, 227773, 227774, 227775, 227776, 227777, 227778, 227779, 227780, 227781, 227782, 227783, 227784, 227785, 227786, 227787, 227788, 227789, 227790, 227791, 227792, 227793, 227794) c6( ) c7(bM, T, yk, oe, ED, cz, aj, j, Ea, V, q, ad, pN, aT, dL, an, hH, xa, KC, xQ, Y, ff, cr, r, aXL, en, g, t, cM, fq, ik, W, ac, lf, IB); #c1(CSMD2) c2(XP_011538874) c3(CUB and sushi domain-containing protein 2 isoform X1) c4(336958, 336959) c5(336960, 336961, 336962, 336963, 336964, 336965, 336966, 336967) c6( ) c7(hb, Y, gZ, n, fe, Fc); #c1(CSMD3) c2(NP_443132) c3(CUB and sushi domain-containing protein 3 isoform 3) c4(229582) c5(229583, 229584, 229585, 229586, 229587, 229588) c6( ) c7(r, aj, fr, nA, IB, bz, q, bN); #c1(CSMD3) c2(NP_937756) c3(CUB and sushi domain-containing protein 3 isoform 1) c4(259035, 259036) c5(259037, 259038, 259039, 259040, 259041, 259042) c6( ) c7(r, aj, fr, nA, lB, bz, q, bN); #c1(CSMD3) c2(NP_937757) c3(CUB and sushi domain-containing protein 3 isoform 2) c4(259043) c5(259044, 259045, 259046, 259047, 259048, 259049) c6( ) c7(r, aj, fr, nA, lB, bz, q, bN); #c1(CSMD3) c2(XP_011515117) c3(CUB and sushi domain-containing protein 3 isoform X8) c4( ) c5(307978) c6( ) c7(r, aj, fr, nA, lB, bz, q, bN); #c1(CSMD3) c2(XP_011515118) c3(CUB and sushi domain-containing protein 3 isoform X9) c4( ) c5(307979, 307980, 307981, 307982, 307983, 307984) c6( ) c7(r, aj, fr, nA, lB, bz, q, bN); #c1(CSN1S1) c2(NP_001020275) c3(alpha-SI-casein isoform 2 precursor) c4(28511) c5(28512, 28513, 28514, 28515, 28516, 28517, 28518, 28519) c6(28520, 28521) c7(aW, aT); #c1(CSN1S1) c2(NP_001881) c3(alpha-SI-casein isoform 1 precursor) c4(121960) c5(121961, 121962, 121963, 121964, 121965, 121966, 121967, 121968) c6(121969, 121970) c7(aW, aT); #c1(CSN1S1) c2(XP_006714153) c3(alpha-Si-casein isoform X2) c4(287600, 287601) c5(287602, 287603, 287604, 287605, 287606, 287607, 287608, 287609, 287610, 287611, 287612, 287613, 287614, 287615, 287616, 287617) c6(287618, 287619, 287620, 287621) c7(aW, aT); #c1(CSN2) c2(XP_011529920) c3(beta-casein isoform X1) c4(325702) c5(325703, 325704, 325705, 325706, 325707, 325708) c6( ) c7(x, A, zQ, aW, hF, aT, aj, j, k, ci, zP, bt, xY, J); #c1(CSN3) c2(NP_005203) c3(kappa-casein precursor) c4( ) c5(153879, 153880, 153881, 153882, 153883) c6( ) c7(gk, aAd, zQ, QK, KE, hb, FB, al, eF, eH, aw, nf, UR, aAe, ci, nA, aha, hu, zP, V, B); #c1(CSN3) c2(XP_011529921) c3 (kappa-casein isoform X2) c4(325709) c5(325710) c6( ) c7(gk, aAd, zQ, QK, KE, hb, FB, al, eF, eH, aw, nf, UR, aAe, ci, nA, aha, hu, zP, V, B); #c1(CSNK1A1) c2(NP_001020276) c3(casein kinase 1 isoform alpha isoform 1) c4(28522) c5(28523, 28524, 28525, 28526, 28527, 28528) c6( ) c7(bE, Y, Az, r, aT, bB, q, X, cp, vi); #c1(CSNK1A1) c2(NP_001258670) c3(casein kinase 1 isoform alpha isoform 3) c4(96306) c5(96307, 96308, 96309, 96310, 96311, 96312, 96313, 96314, 96315, 96316) c6( ) c7(bE, Y, Az, r, aT, bB, q, X, cp, vi); #c1(CSNK1A1) c2(NP_001883) c3(casein kinase 1 isoform alpha isoform 2) c4(121971) c5(121972, 121973, 121974, 121975, 121976, 121977) c6( ) c7(bE, Y, Az, r, aT, bB, q, X, cp, vi); #c1(CSNK1D) c2(NP_001884) c3(casein kinase 1 isoform delta isoform 1) c4(121978, 121979) c5(121980, 121981, 121982, 121983, 121984, 121985, 121986, 121987, 121988, 121989, 121990, 121991) c6( ) c7(aXM, aXN, s, yE, aW, cr, aT, aA, ayp, aXO, aj, bK, K, ur, uu, azg, j, nJ, af); #c1(CSNK1D) c2(NP_620693) c3(casein kinase 1 isoform delta isoform 2) c4(238052) c5(238053, 238054, 238055, 238056, 238057, 238058, 238059, 238060, 238061, 238062, 238063, 238064) c6( ) c7(aXM, aXN, s, yE, aW, cr, aT, aA, ayp, aXO, aj, bK, K, ur, uu, azg, j, nJ, af); #c1(CSNK1D) c2(XP_005256393) c3(casein kinase 1 isoform delta isoform X1) c4(274169) c5(274/70, 274/71, 274/72, 274/73, 274/74, 274175) c6( ) c7(aXM, aXN, s, yE, aW, cr, aT, aA, ayp, aXO, aj, bK, K, ur, uu, azg, j, nJ, af); #c1(CSNK1D) c2(XP_005256394) c3(casein kinase 1 isoform delta isoform X2) c4(274/76) c5( ) c6( ) c7(aXM, aXN, s, yE, aW, cr, aT, aA, ayp, aXO, aj, bk, K, ur, uu, azg, j, nJ, af); #c1(CSNK1G3) c2(NP_001026982) c3(casein kinase 1 isoform gamma-3 isoform 2) c4(30044) c5(30045, 30046, 30047, 30048, 30049, 30050, 30051, 30052, 30053, 30054) c6( ) c7(c); #c1(CSNK1G3) c2(NP_001038188) c3(casein kinase 1 isoform gamma-3 isoform 4) c4(34/71) c5(34172, 34173, 34174, 34175, 34/76, 34177, 34178, 34179, 34180, 34181) c6( ) c7(c); #c1(CSNK1G3) c2(NP_001257501) c3(casein kinase 1 isoform gamma-3 isoform 5) c4(95498) c5(95499, 95500, 95501, 95502, 95503, 95504) c6( ) c7(c); #c1(CSNK1G3) c2(NP_004375) c3(casein kinase 1 isoform gamma-3 isoform 1) c4(145892) c5(145893, 145894, 145895, 145896, 145897, 145898, 145899, 145900, 145901, 145902) c6( ) c7(c); #c1(CSNK2A1) c2(XP_011527477) c3(casein kinase II subunit alpha isoform X1) c4(322244, 322245, 322246, 322247) c5(322248, 322249, 322250, 322251, 322252, 322253, 322254, 322255, 322256, 322257, 322258, 322259, 322260, 322261, 322262, 322263, 322264, 322265, 322266, 322267, 322268, 322269, 322270) c6( ) c7(T, aXP, fd, bB, q, er, ad, aE, aT, K, ae, d, aW, ff, CT, Xg, r, en, zE, bE, hk, Qu, bt, kS); #c1(CSNK2A1) c2(XP_011527478) c3(casein kinase II subunit alpha isoform X2) c4( ) c5(322271, 322272, 322273, 322274, 322275) c6( ) c7(T, aXP, fd, bB, q, er, ad, aE, aT, K, ae, d, aW, ff, CT, Xg, r, en, zE, bE, hk, Qu, bt, kS); #c1(CSNK2A2) c2(NP_001887) c3(casein kinase 11 subunit alpha’) c4(121992) c5(121993, 121994, 121995, 121996, 121997, 121998) c6( ) c7(bF, d, aXP, Qu, hk, ff, r, en, q); #c1(CSNK2B) c2(NP_001311) c3(casein kinase II subunit beta isoform 1) c4(116609, 116610, 116611) c5(116612, 116613, 116614, 116615, 116616, 116617, 116618, 116619, 116620, 116621, 116622, 116623, 116624, 116625, 116626, 116627, 116628, 116629, 116630, 116631, 116632, 116633, 116634, 116635, 116636, 116637, 116638, 116639, 116640, 116641, 116642, 116643, 116644, 116645, 116646, 116647, 116648, 116649, 116650, 116651, 116652, 116653, 116654, 116655, 116656, 116657, 116658, 116659, 116660, 116661, 116662, 116663, 116664, 116665, 116666, 116667, 116668, 116669, 116670, 116671, 116672, 116673, 116674, 116675, 116676, 116677, 116678, 116679, 116680, 116681, 116682, 116683, 116684, 116685, 116686, 116687, 116688, 116689, 116690, 116691, 116692, 116693, 116694, 116695, 116696, 116697, 116698, 116699, 116700, 116701, 116702, 116703, 116704, 116705, 116706, 116707, 116708, 116709, 116710, 116711, 116712, 116713, 116714, 116715, 116716, 116717, 116718, 116719, 116720, 116721, 116722, 116723, 116724, 116725, 116726, 116727, 116728, 116729, 116730, 116731, 116732, 116733, 116734, 116735, 116736) c6( ) c7(bF, hk, ff, hL, eZ, en, y, B); #c1(CSPG4) c2(NP_001888) c3(chondroitin sulfate proteoglycan 4 precursor) c4(121999, 122000) c5(122001, 122002, 122003, 122004, 122005, 122006, 122007, 122008, 122009, 122010, 122011, 122012) c6( ) c7(T, d, Dy, ac, aW, Y, aT, lG, arw, k, aCp, af, eu); #c1(CSPG5) c2(NP_001193871) c3(chondroitin sulfate proteoglycan 5 isoform 2) c4( ) c5(82885, 82886, 82887, 82888, 82889) c6( ) c7(AH, Eg); #c1 (CSPG5) c2(NP_001193872) c3(chondroitin sulfate proteoglycan 5 isoform 3 precursor) c4(82890) c5(82891, 82892, 82893, 82894, 82895, 82896) c6( ) c7(AH, Eg); #c1(CSPG5) c2(NP_006565) c3(chondroitin sulfate proteoglycan 5 isoform 1 precursor) c4(166273, 166274) c5(166275, 166276, 166277, 166278, 166279, 166280, 166281) c6( ) c7(AH, Eg); #c1(CSPP1) c2(NP_001278268) c3(centrosome and spindle pole-associated protein 1 isoform 2) c4( ) c5(109537, 109538, 109539, 109540, 109541, 109542, 109543, 109544) c6( ) c7(aXQ, Ng); #c1(CSPP1) c2(NP_079066) c3(centrosome and spindle pole-associated protein 1 isoform 1) c4(216863) c5(216864, 216865, 216866, 216867, 216868) c6( ) c7(aXQ, Ng); #c1(CSPP1) c2(XP_005251362) c3(centrosome and spindle pole-associated protein 1 isoform X3) c4(270921) c5(270922, 270923, 270924, 270925, 270926, 270927) c6( ) c7(aXQ, Ng); #c1(CSPP1) c2(XP_006716537) c3(centrosome and spindle pole-associated protein 1 isoform X7) c4(290377) c5(290378, 290379) c6( ) c7(aXQ, Ng); #c1(CSPP1) c2(XP_011515904) c3(centrosome and spindle pole-associated protein 1 isoform X6) c4( ) c5(309066) c6( ) c7(aXQ, Ng); #c1(CSPP1) c2(XP_011515912) c3(centrosome and spindle pole-associated protein 1 isoform X13) c4( ) c5(309067) c6( ) c7(aXQ, Ng); #c1(CSRNP1) c2(NP_149016) c3(cysteine/serine-rich nuclear protein 1) c4(226729, 226730) c5(226731, 226732, 226733, 226734, 226735, 226736, 226737, 226738, 226739, 226740, 226741, 226742, 226743, 226744, 226745, 226746) c6( ) c7(bB, aj, ci, mx); #c1(CSRNP3) c2(NP_079245) c3(cysteine/serine-rich nuclear protein 3) c4(217573) c5(217574, 217575, 217576, 217577, 217578, 217579, 217580, 217581, 217582, 217583, 217584, 217585, 217586, 217587, 217588, 217589, 217590, 217591) c6( ) c7(IB, bN); #c1(CSRNP3) c2(XP_005246922) c3(cysteine/serine-rich nuclear protein 3 isoform X1) c4(267495, 267496, 267497, 267498) c5(267499, 267500, 267501, 267502, 267503, 267504, 267505, 267506, 267507, 267508, 267509, 267510, 267511, 267512, 267513, 267514) c6( ) c7(IB, bN); #c1(CSRP1) c2(NP_001180500) c3(cysteine and glycine-rich protein 1 isoform 1) c4(74166, 74167) c5(74168, 74169, 74170, 74171, 74172, 74173, 74174, 74175, 74/76, 74177, 74178, 74/79, 74180, 74181, 74182, 74183, 74184, 74185, 74186, 74187, 74188, 74189, 74190, 74191, 74192) c6( ) c7(P, gV, T, bL, dD, bM, pf, atB, fG, e, ea, cz, it, n, aj, bS, j, ct, C, gf, v, eq, q, J, ad, ai, aMB, fU, jz, Zu, oz, afn, ds, M, Os, aT, cy, h, JK, K, ae, vl, hm, A, mR, hG, aW, aEH, ff, eH, hl, eZ, r, fe, aq, vs, en, iV, Z, eF, bz, qa, LB, eY, tW, W, ac, bE, aWe, eM, dh, mx, eB, at, MJ, wx, aVY, fD, Lz, ei, y, xg, B, L); #c1(CSRP2) c2(NP_001287894) c3(cysteine and glycine-rich protein 2) c4(113166) c5(113167, 113168, 113169, 113170, 113171, 113172, 113173, 113174, 113175, 113176, 113177) c6( ) c7(aW, aT); #c1(CSRP2BP) c2(NP_065397) c3(cysteine-rich protein 2-binding protein) c4(204742, 204743) c5(204744, 204745, 204746, 204747, 204748, 204749, 204750, 204751, 204752, 204753, 204754, 204755, 204756, 204757, 204758) c6( ) c7(aSP); #c1(CSRP3) c2(NP_003467) c3(cysteine and glycine-rich protein 3) c4(138571) c5(138572, 138573, 138574, 138575, 138576, 138577, 138578, 138579, 138580, 138581) c6( ) c7(azs, T, tc, v, aml, jE, ci, cd, ad, Na, bv, cc, ib, aE, aXS, amJ, aT, vc, Nc, K, Tj, ae, aW, ff, eH, Pg, aXR, en, mo, eF, t, cm, pm, Bt, ac, eM, mx, ll, nw, lB); #c1(CST1) c2(NP_001889) c3(cystatin-SN precursor) c4(122013) c5(122014, 122015, 122016, 122017, 122018, 122019, 122020, 122021, 122022, 122023) c6( ) c7(bL, bE, r, aA, ahr, bB, aF, q, X, ad); #c1(CST2) c2(NP_001313) c3(cystatin-SA precursor) c4(116737) c5(116738, 116739, 116740, 116741, 116742, 116743) c6( ) c7(aXT, Wh, ac, nv, m, R, t, H, iK, af, y); #c1(CST3) c2(NP_001275543) c3(cystatin-C precursor) c4(106827, 106828) c5(106829, 106830, 106831, 106832, 106833, 106834, 106835, 106836, 106837, 106838, 106839, 106840, 106841, 106842) c6( ) c7(T, aXW, ea, e, l, aR, ip, aao, x, aXV, Ex, H, hm, eG, aW, hl, as, sz, aBO, U, R, dD, gU, aw, cQ, aj, PJ, fK, cd, ke, BI, Hx, yZ, Um, aXT, aXU, cM, W, dB, aXX, FV, bm, at, E, bL, eR, cz, GC, v, j, yU, V, J, WY, rS, aT, qG, an, ae, af, bA, ak, bf, iU, aaE, lo, aaQ, wA, iK, tj, ac, c, yW, dh, dy, bt, aDj, aaQ, eq, ad, fU, sO, Dq, acQ, sj, d, azS, lU, cW, bz, dr, dJ, kq, aS, y, us); #c1(CST4) c2(NP_001890) c3(cystatin-S precursor) c4(122024, (22025) c5(122026, 122027, 122028, 122029, 122030, 122031) c6( ) c7(aAV); #c1(CST5) c2(NP_001891) c3(cystatin-D precursor) c4(122032) c5(122033, 122034, 122035, 122036, 122037, 122038) c6( ) c7(aXT, Wh, nv, m, R, t, H, iK, af, ad); #c1(CST6) c2(NP_001314) c3(cystatin-M precursor) c4(116744) c5(116745, 116746, 116747, 116748, 116749, 116750) c6( ) c7(T, aC, Wh, if, ci, qN, eA, ad, nv, aT, lQ, Dz, H, ae, af, aXT, MD, qt, aW, Y, cr, g, mo, eE, t, iK, dr, fk, eM, nB, dB, m, R); #c1(CST7) c2(NP_003641) c3(cystatin-F precursor) c4(139830, 139831) c5(139832, 139833, 139834, 139835, 139836, 139837, 139838, 139839, 139840, 139841, 139842, 139843, 139844, 139845, 139846, 139847) c6(139848, 139849, 139850, 139851) c7(T, aA, aDB, aj, xl, aLY); #c1(CST8) c2(NP_005483) c3(cystatin-8 precursor) c4(156607, 156608) c5(156609, 156610, 156611, 156612, 156613, 156614, 156615, 156616, 156617, 156618, 156619, 156620, 156621, 156622, 156623, 156624) c6(156625, 156626, 156627, 156628) c7(gU, Y, aXY, af); #c1(CST9) c2(NP_001008693) c3(cystatin-9 precursor) c4(23352) c5(23353, 23354, 23355, 23356, 23357, 23358, 23359, 23360) c6(23361, 23362) c7(oO, aW, e, aT, dy, ci, sj); #c1 (CST9L) c2(NP_542177) c3(cystatin-9-like precursor) c4(232191, 232192) c5(232193, 232194, 232195, 232196, 232197, 232198, 232199, 232200, 232201, 232202, 232203, 232204, 232205, 232206, 232207, 232208) c6(232209, 232210, 232211, 232212) c7(e, UA, sj); #c1(CSTA) c2(NP_005204) c3(cystatin-A) c4(153884) c5(153885, 153886, 153887, 153888, 153889, 153890) c6( ) c7(bM, T, aXZ, aj, J, ad, aT, K, ae, aiJ, d, qt, aW, Y, ff, cr, en, g, eE, dw, ik, bz, eM, dB, at, it); #c1(CSTB) c2(NP_000091) c3(cystatin-B) c4(1661) c5(1662, 1663, 1664, 1665, 1666, 1667) c6( ) c7(T, ha, it, iD, ci, yU, aR, ad, ip, Mo, yt, aT, h, yT, yZ, Sq, hu, ae, yr, d, agT, aW, Y, g, as, f, XI, bz, cM, akq, yX, ky, aYa, aYb, aem, aPS, U, KA, Bh, B, vi); #c1(CSTF1) c2(NP_001315) c3(cleavage stimulation factor subunit 1) c4(116751, 116752, 116753, 116754) c5(116755, 116756, 116757, 116758, 116759, 116760, 116761, 116762, 116763, 116764, 116765, 116766, 116767, 116768, 116769, 116770, 116771, 116772, 116773, 116774, 116775, 116776, 116777, 116778, 116779, 116780, 116781, 116782) c6( ) c7(at, dL); #c1(CSTF2) c2(NP_001316) c3(cleavage stimulation factor subunit 2) c4(116783, 116784, 116785) c5(116786, 116787, 116788, 116789, 116790, 116791, 116792, 116793, 116794, 116795, 116796, 116797) c6( ) c7(bz, eM); #c1(CSTF2) c2(XP_011529176) c3(cleavage stimulation factor subunit 2 isoform X2) c4( ) c5(324547, 324548, 324549, 324550, 324551, 324552) c6( ) c7(bz, eM); #c1(CSTF2T) c2(NP_056050) c3(cleavage stimulation factor subunit 2 tau variant) c4(185336) c5(185337, 185338, 185339, 185340, 185341, 185342, 185343, 185344, 185345, 185346, 185347, 185348, 185349, 185350, 185351, 185352, 185353, 185354, 185355, 185356, 185357, 185358, 185359, 185360, 185361, 185362, 185363, 185364, 185365, 185366, 185367, 185368, 185369) c6( ) c7(gU); #c1(CSTL1) c2(NP_612140) c3(cystatin-like 1 precursor) c4(236013, 236014) c5(236015, 236016, 236017, 236018, 236019, 236020, 236021, 236022, 236023, 236024, 236025, 236026, 236027, 236028, 236029, 236030, 236031, 236032, 236033, 236034, 236035) c6(236036, 236037, 236038, 236039, 236040) c7(an); #c1(CT45A1) c2(NP_001017417) c3(cancer/testis antigen family 45 member A1) c4(26471) c5(26472, 26473, 26474, 26475, 26476, 26477) c6( ) c7(T, no, wu, aW, pX, aT, m, g, bu, wm, t); #c1(CT55) c2(NP_001026875) c3(cancer/testis antigen 55 isoform 1 precursor) c4(29778) c5(29779, 29780, 29781, 29782, 29783, 29784) c6( ) c7(os, Ub, aW, aT, oZ, m, t); #c1(CT55) c2(NP_060333) c3(cancer/testis antigen 55 isoform 2 precursor) c4(195428) c5(195429, 195430, 195431, 195432, 195433, 195434) c6( ) c7(os, Ub, aW, aT, oZ, m, t); #c1(CT83) c2(NP_001017978) c3(kita-kyushu lung cancer antigen 1) c4(26710) c5(26711, 26712, 26713, 26714, 26715, 26716) c6( ) c7(ra, eM, ff, bu, g, en, bz, ad); #c1 (CTAG1A) c2(NP_640343) c3(cancer/testis antigen 1) c4(238472) c5(238473, 238474, 238475, 238476, 238477, 238478, 238479, 238480, 238481, 238482) c6( ) c7(T, id, wu, if, ht, al, jh, fd, n, ze, qN, AO, iJ, q, ad, eA, aHy, iu, Bl, ib, WB, nv, aT, h, wm, hu, is, fb, ae, afu, je, iU, nO, aW, Y, Lf, AQ, ff, r, en, bu, g, t, cm, bz, ck, iM, Of, eM, iT, au, m, bt, it, B, LS, vi); #c1(CTAG2) c2(NP_066274) c3(cancer/testis antigen 2 isoform LAGE-1b) c4(206898) c5(206899, 206900, 206901, 206902, 206903, 206904) c6( ) c7(T, id, Y, au, ht, ff, bu, r, aA, it, fd, m, en, hu, g, t, aUy, ae, q, ad); #c1(CTAG2) c2(NP_758965) c3(cancer/testis antigen 2 isoform LAGE-la) c4(249426, 249427) c5(249428, 249429, 249430, 249431, 249432, 249433, 249434, 249435, 249436, 249437, 249438, 249439) c6( ) c7(T, id, Y, au, ht, ff, bu, r, aA, it, fd, m, en, hu, g, t, aUy, ae, q, ad); #c1(CTAGE1) c2(NP_758441) c3(cTAGE family member 2) c4(249160) c5(249161, 249162, 249163, 249164, 249165, 249166) c6( ) c7(hb, T); #c1(CTAGE5) c2(NP_001234918) c3(cTAGE family member 5 isoform 6) c4(87767) c5(87768, 87769, 87770, 87771, 87772, 87773) c6( ) c7(bE, T, Y, ip, aYc); #c1(CTAGE5) c2(NP_005921) c3(cTAGE family member 5 isoform 1) c4(160378, 160379) c5(160380, 160381, 160382, 160383, 160384, 160385, 160386, 160387, 160388, 160389, 160390, 160391, 160392) c6( ) c7(bE, T, Y, ip, aYc); #c1 (CTAGE5) c2(NP_976229) c3(cTAGE family member 5 isoform 2) c4(263014, 263015, 263016) c5(263017, 263018, 263019, 263020, 263021, 263022, 263023, 263024, 263025, 263026, 263027, 263028) c6( ) c7(bE, T, Y, ip, aYc); #c1(CTAGE5) c2(NP_976230) c3(cTAGE family member 5 isoform 3) c4(263029) c5(263030, 263031, 263032, 263033, 263034, 263035) c6( ) c7(bE, T, Y, ip, aYc); #c1(CTAGE5) c2(NP_976231) c3(cTAGE family member 5 isoform 4) c4( ) c5(263036, 263037, 263038, 263039, 263040) c6( ) c7(bE, T, Y, ip, aYc); #c1(CTBP1) c2(NP_001012632) c3(C-terminal-binding protein 1 isoform 2) c4(25101) c5(25102, 25103, 25104, 25105, 25106, 25107, 25108, 25109, 25110, 25111, 25112, 25113, 25114, 25115, 25116, 25117, 25118, 25119) c6( ) c7(bF, T, os, ac, fR, s, aW, Y, aT, r, aA, oZ, v, hR, ci, aF, bw, ae, q, ad); #c1(CTBP1) c2(NP_001319) c3(C-terminal-binding protein 1 isoform 1) c4(116798) c5(116799, 116800, 116801, 116802, 116803, 116804) c6( ) c7(bF, T, os, ac, fR, s, aW, Y, aT, r, aA, oZ, v, hR, ci, af, bw, ae, q, ad); #c1(CTBP1) c2(XP_011511698) c3(C-terminal-binding protein 1 isoform X2) c4(302430, 302431) c5( ) c6( ) c7(bF, T, os, ac, fR, s, aW, Y, aT, r, aA, oZ, v, hR, ci, aF, bw, ae, q, ad); #c1(CTBP2) c2(NP_001277143) c3(C-terminal-binding protein 2 isoform 1) c4(109122) c5(109123, 109124, 109125, 109126, 109127, 109128, 109129, 109130, 109131, 109132, 109133, 109134, 109135, 109136, 109137, 109138, 109139, 109140) c6( ) c7(ck, T, aW, hL, aT, m, cl, t, it, ad); #c1(CTBP2) c2(NP_073713) c3(C-terminal-binding protein 2 isoform 2) c4(213192, 213193) c5(213194, 213195, 213196, 213197, 213198, 213199) c6( ) c7(ck, T, aW, hL, aT, m, cl, t, it, ad); #c1(CTBP2) c2(XP_011537651) c3(C-terminal-binding protein 2 isoform X2) c4( ) c5(335216) c6( ) c7(ck, T, aW, hl, aT, m, cl, t, it, ad); #c1(CTBP2) c2(XP_011537660) c3(C-terminal-binding protein 2 isoform X3) c4( ) c5(335217, 335218, 335219, 335220, 335221) c6( ) c7(ck, T, aW, hL, aT, m, cl, t, it, ad); #c1(CTC1) c2(NP_079375) c3(CST complex subunit CTC1) c4(217848) c5(217849, 217850, 217851, 217852, 217853, 217854) c6( ) c7(sb, hb, ajw, lS, aYe, aYd); #c1(CTC1) c2(XP_011522312) c3(CST complex subunit CTC1 isoform X1) c4(317009) c5(317010, 317011, 317012, 317013, 317014, 317015) c6( ) c7(sb, hb, ajw, lS, aYe, aYd); #c1(CTCF) c2(NP_001177951) c3(transcriptional repressor CTCF isoform 2) c4(73230) c5(73231, 73232, 73233, 73234, 73235) c6( ) c7(cZ, T, fR, ml, aMv, j, ci, nA, AD, aYh, q, ad, er, eW, x, av, aT, aYf, dL, dd, he, eL, qw, je, ND, vT, qt, aW, ff, az, LO, eH, r, Ej, Ku, bu, en, t, cl, bz, eF, dr, dR, ck, wT, iM, bP, aYg, hL, aNL, rx, k, m, ei, bt); #c1(CTCF) c2(NP_006556) c3(transcriptional repressor CTCF isoform 1) c4(166220) c5(166221, 166222, 166223, 166224, 166225, 166226, 166227, 166228) c6( ) c7(cZ, T, FR, ml, aMv, j, ci, nA, AO, aYh, q, ad, er, eW, x, av, aT, aYf, dl, dd, he, eL, qw, je, ND, vT, qt, aW, ff, az, LO, eH, r, Ej, Ku, bu, en, t, cl, bz, eF, dr, dR, ck, wT, iM, bP, aYg, hL, aNL, rx, k, m, ei, bt); #c1(CTCFL) c2(NP_001255970) c3 (transcriptional repressor CTCFL isoform 1) c4(94996) c5(94997, 94998, 94999, 95000, 95001, 95002, 95003, 95004, 95005, 95006, 95007) c6( ) c7(T, aYi, ht, n, ci, x, av, hw, TK, aT, hu, eL, vT, aW, Y, ff, LO, Qm, en, bu, cl, t, ik, bz, wT, bE, hL, np, m, k); #c1(CTCFL) c2(NP_001255972) c3(transcriptional repressor CTCFL isoform 3) c4(95008, 95009) c5(95010, 95011, 95012, 95013, 95014, 95015) c6( ) c7(T, aYi, ht, n, ci, x, av, hw, TK, aT, hu, eL, vT, aW, Y, ff, LO, Qm, en, bu, cl, t, ik, bz, wT, bE, hL, np, m, k); #c1 (CTCFL) c2(NP_001255973) c3 (transcriptional repressor CTCFL isoform 4) c4(95016, 95017) c5(95018, 95019, 95020, 95021, 95022, 95023) c6( ) c7(T, aYi, ht, n, ci, x, av, hw, TK, aT, hu, eL, vT, aW, Y, ff, LO, Am, en, bu, cl, t, ik, bz, wT, bE, hl, np, m, k); #c1(CTCFL) c2(NP_001255974) c3(transcriptional repressor CTCFL isoform 5) c4(95024) c5(95025, 95026) c6( ) c7(T, aYi, ht, n, ci, x, av, hw, TK, aT, hu, eL, vT, aW, Y, ff, LO, Qm, en, bu, cl, t, ik, bz, wT, bE, hl, np, m, k); #c1 (CTCFL) c2(NP_001255975) c3(transcriptional repressor CTCFL isoform 6) c4(95027, 95028) c5(95029, 95030, 95031, 95032, 95033, 95034) c6( ) c7(T, aYi, ht, n, ci, x, av, hw, TK, aT, hu, eL, vT, aW, Y, ff, LO, Qm, en, bu, cl, t, ik, bz, wT, bE, hL, np, m, k); #c1(CTCFL) c2(NP_001255976) c3(transcriptional repressor CTCFL isoform 7) c4(95035) c5(95036, 95037, 95038, 95039, 95040, 95041, 95042, 95043, 95044, 95045, 95046, 95047, 95048, 95049, 95050, 95051, 95052) c6( ) c7(T, aYi, ht, n, ci, x, av, hw, TK, aT, hu, el, vT, aW, Y, ff, LO, Qm, en, bu, cl, t, ik, bz, wT, bE, hl, np, m, k); #c1(CTCFL) c2(NP_001255977) c3 (transcriptional repressor CTCFL isoform 8) c4(95053) c5(95054, 95055, 95056, 95057, 95058, 95059) c6( ) c7(T, aYi, ht, n, ci, x, av, hw, TK, aT, hu, eL, vT, aW, Y, ff, LO, Qm, en, bu, cl, t, ik, bz, wT, bE, hl, np, m, k); #c1(CTCFL) c2(NP_001255978) c3(transcriptional repressor CTCFL isoform 9) c4( ) c5(95060) c6( ) c7(T, aYi, ht, n, ci, x, av, hw, TK, aT, hu, eL, vT, aW, Y, ff, LO, Qm, en, bu, cl, t, ik, bz, wT, bE, hL, np, m, k); #c1(CTCFL) c2(NP_001255979) c3(transcriptional repressor CTCFL isoform 10) c4( ) c5(95061, 95062, 95063, 95064, 95065, 95066, 95067, 95068) c6( ) c7(T, aYi, ht, n, ci, x, av, hw, TK, aT, hu, eL, vT, aW, Y, ff, LO, Qm, en, bu, cl, t, ik, bz, wT, bE, hl, np, m, k); #c1(CTCFL) c2(NP_001255983) c3(transcriptional repressor CTCFL isoform 12) c4( ) c5(95069, 95070, 95071, 95072, 95073) c6( ) c7(T, aYi, ht, n, ci, x, av, hw, TK, aT, hu, eL, vT, aW, Y, ff, LO, Qm, en, bu, cl, t, ik, bz, wT, bE, hl, np, m, k); #c1(CTDP1) c2(NP_004706) c3(RNA polymerase II subunit A C-terminal domain phosphatase isoform 1) c4(148967) c5(148968, 148969, 148970, 148971, 148972, 148973) c6( ) c7(agU, ad, bk, aYj, c); #c1(CTDP1) c2(NP_430255) c3(RNA polymerase II subunit A C-terminal domain phosphatase isoform 2) c4(229075) c5(229076, 229077, 229078, 229079, 229080, 229081, 229082, 229083) c6( ) c7(agU, ad, bK, aYj, c); #c1(CTDSP1) c2(NP_001193807) c3(carboxy-terminal domain RNA polymerase II polypeptide A small phosphatase 1 isoform 3) c4(82626) c5(82627, 82628, 82629, 82630, 82631, 82632) c6( ) c7(T, iU, iT, WB, aW, aT, fd, bu); #c1(CTDSP1) c2(NP_067021) c3(carboxy-terminal domain RNA polymerase II polypeptide A small phosphatase 1 isoform 1) c4(208448) c5(208449, 208450, 208451, 208452, 208453, 208454) c6( ) c7(T, iU, iT, WB, aW, aT, fd, bu); #c1 (CTDSP2) c2(NP_005721) c3(carboxy-terminal domain RNA polymerase II polypeptide A small phosphatase 2) c4(158822) c5(158823, 158824, 158825, 158826, 158827, 158828) c6( ) c7(af); #c1(CTDSPL) c2(NP_001008393) c3(CTD small phosphatase-like protein isoform 1) c4(23146) c5(23147, 23148, 23149, 23150, 23151, 23152) c6( ) c7(es, T, av, eM, iT, aW, ff, ik, aT, hn, fr, ci, en, qN, g, bt, ad); #c1(CTDSPL) c2(NP_005799) c3(CTD small phosphatase-like protein isoform 2) c4(159553) c5(159554, 159555, 159556, 159557, 159558, 159559) c6( ) c7(es, T, av, eM, iT, aW, ff, ik, aT, hn, fr, ci, en, qN, g, bt, ad); #c1(CTF1) c2(NP_001136016) c3(cardiotrophin-1 isoform 2) c4(54012) c5(54013, 54014, 54015, 54016, 54017, 54018) c6( ) c7(bL, dD, lT, Ol, ea, e, cz, v, eq, cd, Ae, bl, K, yo, cH, JV, aq, uU, kk, qa, c, mx, U, gZ, dy, ei, B, L); #c1(CTF1) c2(NP_001321) c3(cardiotrophin-1 isoform 1) c4(116805) c5(116806, 116807, 116808, 116809, 116810, 116811, 116812, 116813, 116814, 116815, 116816, 116817, 116818, 116819, 116820, 116821, 116822, 116823, 116824, 116825, 116826, 116827, 116828, 116829, 116830, 116831, 116832, 116833, 116834, 116835, 116836, 116837, 116838, 116839, 116840, 116841, 116842, 116843, 116844, 116845, 116846, 116847, 116848, 116849, 116850, 116851, 116852, 116853, 116854, 116855, 116856, 116857, 116858, 116859, 116860, 116861, 116862, 116863, 116864, 116865, 116866, 116867, 116868, 116869, 116870, 116871, 116872, 116873, 116874, 116875, 116876, 116877, 116878, 116879, 116880, 116881, 116882, 116883, 116884, 116885, 116886, 116887, 116888, 116889, 116890, 116891, 116892, 116893, 116894, 116895, 116896, 116897) c6( ) c7(bL, dD, lT, Ol, ea, e, cz, v, eq, cd, Ae, bl, K, yo, cH, JV, aq, ul, kk, qa, c, mx, U, gZ, dy, ei, B, L); #c1 (CTGF) c2(NP_001892) c3(connective tissue growth factor precursor) c4(122039) c5(122040, 122041, 122042, 122043, 122044, 122045) c6( ) c7(oO, bM, T, aC, ea, if, al, fd, lR, ci, Lw, aY, ip, x, aE, ws, bW, aYm, atD, K, bc, cp, aa, li, eG, dH, aW, hl, g, eF, ou, eM, nB, vA, vy, uJ, wx, k, Hb, m, fy, iu, FW, dD, pG, aw, n, cQ, aj, fr, uE, qN, cd, ai, zw, eV, aNj, ey, pj, hB, Ha, Dg, A, aiB, anP, EU, fQ, aq, fe, kp, dw, aYo, op, vD, at, it, B, vQ, bL, fM, eR, DD, fG, oe, jx, pQ, eX, aA, cz, fF, v, j, vp, aGK, q, J, er, eA, aT, cy, aLM, bq, an, lM, af, hM, bf, Y, r, wl, aYp, lo, en, t, iK, pb, ck, aYk, sS, QG, DW, dh, aYl, dy, Gg, hS, kh, MH, uG, aNk, jh, dG, eq, jE, hX, dP, fU, av, hd, F, ib, sN, io, vw, Dj, vB, ji, amY, iL, d, lR, ff, xs, CH, Ce, aF, hp, Fq, iV, bz, LB, gk, Ew, gZ, Hs, lC, X, y, aYn); #c1(CTHRC1) c2(NP_001243028) c3(collagen triple helix repeat-containing protein 1 isoform 2) c4(89625, 89626) c5(89627, 89628, 89629, 89630, 89631, 89632, 89633, 89634, 89635, 89636, 89637, 89638) c6( ) c7(T, pf, aA, bB, n, ci, Rk, ai, q, aE, aT, cp, VC, aW, Y, ff, xs, LZ, r, aF, en, bE, y, B); #c1(CTHRC1) c2(NP_612464) c3(collagen triple helix repeat-containing protein 1 isoform 1 precursor) c4(236578) c5(236579, 236580, 236581, 236582, 236583, 236584) c6( ) c7(T, pf, aA, bB, n, ci, Rk, ai, q, aE, aT, cp, VC, aW, Y, ff, xs, LZ, r, aF, en, bE, y, B); #c1(CTIF) c2(NP_001135869) c3(CBP80/20-dependent translation initiation factor isoform 2) c4(53441) c5(53442, 53443, 53444, 53445, 53446, 53447) c6( ) c7(aC, an); #c1(CTIF) c2(NP_055587) c3(CBP80/20-dependent translation initiation factor isoform 1) c4(182137, 182138) c5(182139, 182140, 182141, 182142, 182143, 182144) c6( ) c7(aC, an); #c1(CTIF) c2(XP_006722649) c3(CBP80/20-dependent translation initiation factor isoform X4) c4( ) c5(294789, 294790, 294791, 294792, 294793, 294794) c6( ) c7(aC, an); #c1(CTLA4) c2(NP_001032720) c3(cytotoxic T-lymphocyte protein 4 isoform CTLA-4delTM precursor) c4(31418, 31419, 31420, 31421) c5(31422, 31423, 31424, 31425, 31426, 31427, 31428, 31429, 31430, 31431, 31432, 31433, 31434, 31435, 31436, 31437, 31438, 31439) c6( ) c7(oO, bM, T, FR, ea, if, al, fd, cL, ur, ci, qj, aFV, Jx, aY, bh, Ap, Tz, lR, x, AW, xy, xE, aE, rw, aYy, Ds, K, wt, DY, hH, bc, cp, hm, zQ, aW, Oj, axu, HA, hl, XC, fc, hN, g, aFU, ve, aYA, uC, hJ, eE, eF, bw, ba, y, yl, eM, Jr, Fh, aYw, iT, aDs, mx, cj, uo, k, lS, ul, qR, abh, dD, sQ, Hm, lo, yG, aw, fN, n, aj, oY, fr, aFF, uE, pM, aYv, ml, qN, aHj, ai, aPb, ft, no, cc, aYr, aMj, Ne, aU, h, hn, dL, aFX, OM, he, aDp, OV, mR, CN, anP, A, oo, sp, OX, CM, ge, fe, aq, po, kp, aMJ, mo, kk, jT, cM, vo, Wt, ar, W, aYq, hk, lf, JM, bm, at, uz, aEG, ut, eo, fl, bk, gg, B, cn, bL, Ob, Dw, DM, fM, dA, eR, DD, aYB, dk, fG, yk, jx, aA, hz, j, bX, Do, abC, V, q, J, o, pd, mP, Ig, apJ, ld, afQ, yg, aT, rS, yu, bK, hK, DX, lM, ae, eu, qw, hM, xR, oV, Y, fO, aFT, lC, r, Pg, bg, aHU, lo, aDm, aYz, en, zM, pb, OC, lY, Cv, ck, cT, sS, cl, OW, ac, BU, ig, yW, hc, aGi, AH, hr, aYx, wR, dy, gT, bt, zA, vi, gx, Kd, Ke, dM, axd, iW, jh, auq, dG, oH, jE, fu, uu, eq, wV, pz, aYs, mC, ad, kL, aFW, dP, hX, av, wD, F, fX, ds, qh, oW, hD, JK, ji, lQ, nJ, yF, uQ, DI, lU, qZ, asf, ff, aFE, eH, aYu, cr, Mz, eZ, hp, BG, iV, bz, aYt, TA, es, Bv, axs, Lj, z, gZ, ei, od, hE, X, hv, pC); #c1(CTLA4) c2(NP_005205) c3(cytotoxic T-lymphocyte protein 4 isoform CTLA4-TM precursor) c4(153891) c5(153892, 153893, 153894, 153895, 153896, 153897, 153898, 153899, 153900, 153901, 153902, 153903, 153904, 153905, 153906, 153907, 153908, 153909, 153910, 153911, 153912, 153913, 153914, 153915, 153916, 153917, 153918, 153919, 153920, 153921, 153922, 153923, 153924, 153925, 153926, 153927, 153928, 153929, 153930, 153931, 153932, 153933, 153934, 153935, 153936, 153937, 153938, 153939, 153940, 153941, 153942, 153943, 153944, 153945, 153946, 153947, 153948, 153949, 153950, 153951, 153952, 153953, 153954, 153955, 153956, 153957, 153958, 153959, 153960, 153961, 153962, 153963, 153964, 153965, 153966, 153967, 153968, 153969, 153970, 153971, 153972, 153973, 153974, 153975, 153976, 153977, 153978, 153979, 153980, 153981, 153982, 153983, 153984) c6(153985) c7(oO, bM, T, fR, ea, if, al, fd, cL, ur, ci, qj, aFV, Jx, aY, bh, Op, Tz, lR, x, AW, xy, xE, aE, rw, aYy, Ds, K, wt, DY, hH, bc, cp, hm, zQ, aW, Oj, axu, HA, hl, XC, fc, hN, g, aFU, ve, aYA, uC, hJ, eE, eF, bw, ba, y, yl, eM, Jr, Fh, aYw, iT, aDs, mx, cj, uo, k, lS, ul, qR, abh, dD, sQ, Hm, lo, yG, aw, fN, n, aj, oY, fr, aFF, uE, pM, aYv, ml, qN, alj, ai, aPb, ft, no, cc, aYr, aMj, Ne, aU, h, hn, dL, aFX, OM, he, aDp, OV, mR, CN, anP, A, oo, sp, OX, CM, ge, fe, aq, po, kp, aMJ, mo, kk, jT, cM, vo, Wt, ar, W, aYq, hk, lf, JM, bm, at, uz, aEG, ut, eo, fl, bk, gg, B, cn, bL, Ob, Dw, DM, fM, dA, eR, DD, aYB, dk, fG, yk, jx, aA, hz, j, bX, Qo, abC, V, q, J, o, pd, mP, lg, apJ, ld, afQ, yg, aT, rS, yu, bK, hk, DX, lM, ae, eu, qw, hM, xR, oV, Y, fO, aFT, lC, r, Pg, bg, aHU, lo, aDm, aYz, en, zM, pb, OC, lY, Cv, ck, cT, sS, cl, OW, ac, BU, ig, yW, hc, aGi, AH, hr, aYx, wR, dy, gT, bt, zA, vi, gx, Kd, Ke, dM, axd, iW, jh, auq, dG, oH, jE, fu, uu, eq, wV, pz, aYs, mC, ad, KL, aFW, dP, hX, av, wD, F, fX, ds, qh, oW, hD, JK, ji, lQ, nJ, yF, uQ, DI, lU, qZ, asf, ff, aFE, eH, aYu, cr, Mz, eZ, hp, BG, iV, bz, aYt, TA, es, Bv, axs, Lj, z, gZ, ei, od, hE, X, hv, pC); #c1(CTNNA1) c2(NP_001277236) c3(catenin alpha-1 isoform 2) c4( ) c5(109253, 109254, 109255, 109256, 109257, 109258, 109259, 109260, 109261, 109262, 109263, 109264, 109265, 109266, 109267, 109268, 109269, 109270, 109271, 109272, 109273, 109274, 109275, 109276, 109277, 109278, 109279, 109280, 109281, 109282, 109283, 109284, 109285, 109286, 109287, 109288, 109289, 109290, 109291, 109292, 109293, 109294) c6( ) c7(bM, T, aC, fR, ht, bs, aA, n, bB, ci, q, br, ad, er, eA, av, Vr, pX, aT, dL, ae, eL, d, s, aW, ff, gK, r, dq, aF, en, cl, as, t, bw, ck, bF, bE, m, EY, alm, bt, it); #c1(CTNNA1) c2(NP_001277239) c3(catenin alpha-1 isoform 4 precursor) c4( ) c5(109295, 109296, 109297, 109298, 109299, 109300) c6( ) c7(bM, T, aC, fR, ht, bs, aA, n, bB, ci, q, br, ad, er, eA, av, Vr, pX, aT, dL, ae, eL, d, s, aW, ff, gK, r, dq, aF, en, cl, as, t, bw, ck, bF, bE, m, EY, alm, bt, it); #c1(CTNNA1) c2(NP_001894) c3(catenin alpha-1 isoform 1) c4(122046, 122047, 122048, 122049, 122050, 122051, 122052, 122053, 122054, 122055, 122056, 122057) c5(122058, 122059, 122060, 122061, 122062, 122063, 122064, 122065, 122066, 122067, 122068, 122069, 122070, 122071, 122072, 122073, 122074, 122075, 122076, 122077, 122078, 122079, 122080, 122081) c6( ) c7(bM, T, aC, fR, ht, bs, aA, n, bB, ci, q, br, ad, er, eA, av, Vr, pX, aT, dL, ae, eL, d, s, aW, ff, gK, r, dq, aF, en, cl, as, t, bw, ck, bF, bE, m, EY, alm, bt, it); #c1 (CTNNA2) c2(NP_001158355) c3(catenin alpha-2 isoform 2) c4(63615, 63616) c5(63617, 63618, 63619, 63620, 63621, 63622, 63623, 63624, 63625, 63626, 63627, 63628, 63629, 63630, 63631, 63632) c6( ) c7(aC, oe, aA, bK, jj, ci, ik, fq); #c1(CTNNA2) c2(NP_001269526) c3(catenin alpha-2 isoform 3) c4(101805) c5(101806, 101807, 101808, 101809, 101810, 101811) c6( ) c7(aC, oe, aA, bK, jj, ci, ik, fq); #c1(CTNNA2) c2(NP_001269527) c3(catenin alpha-2 isoform 4) c4(101812) c5(101813, 101814, 101815, 101816, 101817, 101818, 101819, 101820, 101821, 101822) c6( ) c7(aC, oe, aA, bK, jj, ci, ik, fq); #c1(CTNNA2) c2(NP_001269528) c3(catenin alpha-2 isoform 5) c4(101823) c5(101824, 101825, 101826, 101827, 101828, 101829) c6( ) c7(aC, oe, aA, bK, jj, ci, ik, fq); #c1(CTNNA2) c2(NP_004380) c3(catenin alpha-2 isoform 1) c4(145942, 145943) c5(145944, 145945, 145946, 145947, 145948, 145949, 145950, 145951, 145952, 145953, 145954, 145955) c6( ) c7(aC, De, aA, bK, jj, ci, ik, fq); #c1(CTNNA3) c2(NP_001278062) c3(catenin alpha-3 isoform b) c4(109427) c5(109428, 109429, 109430, 109431, 109432, 109433, 109434, 109435, 109436, 109437, 109438, 109439, 109440, 109441, 109442, 109443, 109444, 109445, 109446, 109447) c6( ) c7(bM, T, id, aw, EQ, n, ci, V, q, er, aYC, XY, dL, an, af, anR, eZ, r, dq, wK, mx, lB, rk); #c1(CTNNA3) c2(NP_037398) c3(catenin alpha-3 isoform a) c4( ) c5(175266, 175267, 175268, 175269, 175270, 175271, 175272, 175273, 175274, 175275, 175276, 175277, 175278, 175279, 175280) c6( ) c7(bM, T, id, aw, EQ, n, ci, V, q, er, aYC, XY, dL, an, af, anR, eZ, r, dq, wK, mx, lB, rk); #c1(CTNNA3) c2(XP_005269774) c3(catenin alpha-3 isoform X5) c4(282803, 282804, 282805) c5(282806) c6( ) c7(bM, T, id, aw, EQ, n, ci, V, q, er, aYC, XY, dL, an, af, anR, eZ, r, dq, wK, mx, lB, rk); #c1(CTNNA3) c2(XP_011538029) c3(catenin alpha-3 isoform X4) c4( ) c5(335587) c6( ) c7(bM, T, id, aw, EQ, n, ci, V, q, er, aYC, XY, dL, an, af, anR, eZ, r, dq, wK, mx, lB, rk); #c1(CTNNAL1) c2(NP_001273903) c3(alpha-catulin isoform b) c4(105875, 105876, 105877) c5(105878, 105879, 105880, 105881, 105882, 105883, 105884, 105885, 105886, 105887, 105888, 105889, 105890, 105891, 105892, 105893, 105894, 105895) c6( ) c7(bM, T, cF, aqJ, Y, iB, ff, en); #c1(CTNNAL1) c2(NP_003789) c3(alpha-catulin isoform a) c4(140821, 140822) c5(140823, 140824, 140825, 140826, 140827, 140828, 140829, 140830, 140831, 140832, 140833, 140834) c6( ) c7(bM, T, cF, aqJ, Y, iB, ff, en); #c1(CTNNB1) c2(NP_001091679) c3(catenin beta-1) c4(38674, 38675) c5(38676, 38677, 38678, 38679, 38680, 38681, 38682, 38683, 38684, 38685, 38686, 38687, 38688, 38689, 38690, 38691, 38692, 38693, 38694, 38695, 38696, 38697, 38698, 38699, 38700, 38701, 38702, 38703) c6( ) c7(T, aC, fR, if, al, aEf, fd, bB, ci, aEe, ip, x, afr, lv, iv, K, eL, Qe, yo, aW, Az, hl, g, as, hJ, ve, eF, auD, iA, nB, wK, vA, U, cj, iS, m, YS, iu, ZF, iB, RX, ht, NT, n, oY, aYH, fr, qN, alD, Yc, apG, h, aEo, Gw, dL, bl, hB, aYF, Yl, hi, iw, ge, aq, Hu, yq, pm, cM, auC, op, Ye, iO, YE, dB, hk, Vy, hL, at, B, jp, aA, hy, Do, aYD, q, er, eA, aeZ, mj, BJ, eO, qK, HC, aT, ae, af, jf, s, iU, Y, r, lo, en, cl, t, ik, aYl, ck, sS, nM, Dy, gX, aJS, bt, hS, Gl, aEh, atS, aYE, ad, afb, hX, dP, av, hd, ib, yt, io, Ti, d, BN, BO, aYG, ff, eH, dq, aF, app, iV, iR, aEm, HI, bz, bF, es, gk, pc, bE, Gu, MC, X); #c1(CTNNB1) c2(XP_006713048) c3(catenin beta-1 isoform X3) c4(286986, 286987, 286988) c5(286989, 286990, 286991, 286992, 286993, 286994, 286995, 286996, 286997, 286998, 286999, 287000, 287001, 287002, 287003, 287004, 287005, 287006, 287007) c6( ) c7(T, aC, fR, if, al, aEf, fd, bB, ci, ale, ip, x, afr, lv, iv, K, eL, Qe, yo, aW, Az, hl, g, as, hJ, ve, eF, auD, iA, nB, wK, vA, U, cj, iS, m, YS, iu, ZF, iB, RX, ht, NT, n, oY, aYH, fr, qN, alD, Yc, apG, h, aEo, Gw, dL, bl, hB, aYF, Yl, hi, iw, ge, aq, Hu, yq, pm, cM, auC, op, Ye, iO, YE, dB, hk, Vy, hL, at, B, jp, aA, hy, Do, aYD, q, er, eA, aeZ, mj, BJ, eD, qK, HC, aT, ae, af, jf, s, iU, Y, r, lo, en, cl, t, ik, aYl, ck, sS, nM, Dy, gX, aJS, bt, hS, GI, aEh, atS, aYE, ad, afb, hX, dP, av, hd, ib, yt, io, Ti, d, BN, BO, aYG, ff, eH, dq, aF, app, iV, iR, aEm, HI, bz, bF, es, gk, pc, bE, Gu, MC, X); #c1 (CTNNBL1) c2(NP_001268424) c3(beta-catenin-like protein 1 isoform 2) c4(99899) c5(99900, 99901, 99902, 99903, 99904, 99905, 99906, 99907, 99908, 99909) c6( ) c7(sc, bM, T, aEj, jh, v, fr, ci, q, ad, Hx, ft, vJ, aT, vH, UD, dL, K, jf, Nr, aW, Y, r, po, aF, g, t, Nq, z, cj, EY, B); #c1(CTNNBL1) c2(NP_110517) c3(beta-catenin-like protein 1 isoform 1) c4(220492, 220493) c5(220494, 220495, 220496, 220497, 220498, 220499, 220500, 220501) c6( ) c7(sc, bM, T, aEj, jh, v, fr, ci, q, ad, Hx, ft, vJ, aT, vH, UD, dL, K, jf, Nr, aW, Y, r, po, aF, g, t, Nq, z, cj, EY, B); #c1(CTNNBL1) c2(XP_011527219) c3(beta-catenin-like protein 1 isoform X1) c4( ) c5(321770, 321771) c6( ) c7(sc, bM, T, aEj, jh, v, fr, ci, q, ad, Hx, ft, vJ, aT, vH, UD, dL, K, jf, Nr, aW, Y, r, po, aF, g, t, Nq, z, cj, EY, B); #c1 (CTNND1) c2(NP_001078927) c3(catenin delta-1 isoform (ABC) c4(38012, 38013, 38014, 38015, 38016, 38017) c5(38018, 38019, 38020, 38021, 38022, 38023, 38024, 38025, 38026, 38027, 38028, 38029, 38030) c6( ) c7(T, aC, iW, bB, fr, ci, AO, q, ad, eA, anU, av, iu, jz, aE, aT, h, By, K, zy, ae, dx, aiB, s, qt, aW, Y, vj, ev, MD, ff, r, dN, ks, en, anW, cl, g, HI, bz, ck, bF, eM, bE, anY, hL, iS, np, m, alm, in, B, BA); #c1(CTNND1) c2(NP_001078928) c3(catenin delta-1 isoform (AB) c4(38031, 38032, 38033, 38034, 38035, 38036) c5(38037, 38038, 38039, 38040, 38041, 38042, 38043) c6( ) c7(T, aC, iW, bB, fr, ci, AO, q, ad, eA, anU, av, iu, jz, aE, aT, h, By, K, zy, ae, dx, aiB, s, qt, aW, Y, vj, ev, MD, ff, r, dN, ks, en, anW, cl, g, HI, bz, ck, bF, eM, bE, anY, hL, iS, np, m, alm, in, B, BA); #c1(CTNND1) c2(NP_001078931) c3(catenin delta-1 isoform (A) c4(38044, 38045, 38046, 38047) c5(38048, 38049, 38050, 38051, 38052, 38053, 38054, 38055, 38056, 38057, 38058, 38059, 38060, 38061, 38062, 38063, 38064, 38065, 38066, 38067, 38068, 38069, 38070, 38071, 38072, 38073, 38074, 38075, 38076, 38077, 38078, 38079, 38080, 38081, 38082, 38083, 38084, 38085, 38086, 38087, 38088) c6( ) c7(T, aC, iW, bB, fr, ci, AO, q, ad, eA, anU, av, iu, jz, aE, aT, h, By, K, zy, ae, dx, aiB, s, qt, aW, Y, vj, ev, MD, ff, r, dN, ks, en, anW, cl, g, HI, bz, ck, bF, eM, bE, anY, hL, iS, np, m, alm, in, B, BA); #c1(CTNND1) c2(NP_001078932) c3 (catenin delta-1 isoform 3ABC) c4( ) c5(38089, 38090, 38091, 38092, 38093, 38094, 38095, 38096, 38097, 38098, 38099) c6( ) c7(T, aC, iW, bB, fr, ci, AO, q, ad, eA, anU, av, iu, jz, aE, aT, h, By, K, zy, ae, dx, aiB, s, qt, aW, Y, vj, ev, MD, ff, r, dN, ks, en, anW, cl, g, HI, bz, ck, bF, eM, bE, anY, hL, iS, np, m, alm, in, B, BA); #c1(CTNND1) c2(NP_001078933) c3(catenin delta-1 isoform 3AB) c4( ) c5(38100, 38101, 38102, 38103, 38104, 38105, 38106, 38107, 38108, 38109, 38110) c6( ) c7(T, aC, iW, bB, fr, ci, AO, q, ad, eA, anU, av, iu, jz, aE, aT, h, By, K, zy, ae, dx, aiB, s, qt, aW, Y, vj, ev, MD, ff, r, dN, ks, en, anW, cl, g, HI, bz, ck, bF, eM, bE, anY, hL, iS, np, m, alm, in, B, BA); #c1(CTNND1) c2(NP_001078938) c3(catenin delta-1 isoform 3A) c4( ) c5(38111, 38112, 38113, 38114, 38115, 38116, 38117, 38118, 38119, 38120, 38121, 38122, 38123, 38124, 38125, 38126, 38127, 38128, 38129, 38130, 38131, 38132, 38133, 38134, 38135, 38136) c6( ) c7(T, aC, iW, bB, fr, ci, AO, q, ad, eA, anU, av, iu, jz, aE, aT, h, By, K, zy, ae, dx, aiB, s, qt, aW, Y, vj, ev, MD, ff, r, dN, ks, en, anW, cl, g, HI, bz, ck, bF, eM, bE, anY, hL, iS, np, m, alm, in, B, BA); #c1(CTNND1) c2(NP_001193812) c3(catenin delta-1 isoform 2ABC) c4( ) c5(82640, 82641, 82642, 82643, 82644, 82645) c6( ) c7(T, aC, iW, bB, fr, ci, AO, q, ad, eA, anU, av, iu, jz, aE, aT, h, By, K, zy, ae, dx, aiB, s, qt, aW, Y, vj, ev, MD, ff, r, dN, ks, en, anW, cl, g, HI, bz, ck, bF, eM, bE, anY, hL, iS, np, m, alm, in, B, BA); #c1(CTNND1) c2(NP_001193814) c3(catenin delta-1 isoform (AC) c4(82646, 82647, 82648) c5(82649, 82650, 82651, 82652, 82653, 82654, 82655, 82656, 82657, 82658, 82659, 82660, 82661, 82662, 82663, 82664, 82665, 82666, 82667, 82668, 82669, 82670, 82671, 82672) c6( ) c7(T, aC, iW, bB, fr, ci, AO, q, ad, eA, anU, av, iu, jz, aE, aT, h, By, K, zy, ae, dx, aiB, s, qt, aW, Y, vj, ev, MD, ff, r, dN, ks, en, anW, cl, g, HI, bz, ck, bF, eM, bE, anY, hL, iS, np, m, alm, in, B, BA); #c1(CTNND1) c2(NP_001193815) c3(catenin delta-1 isoform 2AB) c4( ) c5(82673, 82674, 82675, 82676, 82677, 82678) c6( ) c7(T, aC, iW, bB, fr, ci, AO, q, ad, eA, anU, av, iu, jz, aE, aT, h, By, K, zy, ae, dx, aiB, s, qt, aW, Y, vj, ev, MD, ff, r, dN, ks, en, anW, cl, g, HI, bz, ck, bF, eM, bE, anY, hL, iS, np, m, alm, in, B, BA); #c1(CTNND1) c2(NP_001193816) c3(catenin delta-1 isoform 28) c4( ) c5(82679, 82680, 82681, 82682, 82683, 82684, 82685, 82686, 82687, 82688, 82689) c6( ) c7(T, aC, iW, bB, fr, ci, AO, q, ad, eA, anU, av, iu, jz, aE, aT, h, By, K, zy, ae, dx, aiB, s, qt, aW, Y, vj, ev, MD, ff, r, dN, ks, en, anW, cl, g, HI, bz, ck, bF, eM, bE, anY, hL, iS, np, m, alm, in, B, BA); #c1(CTNND1) c2(NP_001193820) c3(catenin delta-1 isoform 2A) c4( ) c5(82690, 82691, 82692, 82693, 82694) c6( ) c7(T, aC, iW, bB, fr, ci, AO, q, ad, eA, anU, av, iu, jz, aE, aT, h, By, K, zy, ae, dx, aiB, s, qt, aW, Y, vj, ev, MD, ff, r, dN, ks, en, anW, cl, g, HI, bz, ck, bF, eM, bE, anY, hL, iS, np, m, alm, in, B, BA); #c1(CTNND1) c2(NP_001322) c3 (catenin delta-l isoform 18) c4(116898, 116899, 116900) c5(116901, 116902, 116903, 116904, 116905, 116906, 116907) c6( ) c7(T, aC, iW, bB, fr, ci, A0, q, ad, eA, anU, av, iu, jz, aE, aT, h, By, K, zy, ae, dx, aiB, s, qt, aW, Y, vj, ev, MD, ff, r, dN, ks, en, anW, cl, g, HI, bz, ck, bF, eM, bE, anY, HL, iS, np, m, alm, in, B, BA); #c1(CTNND2) c2(NP_001275644) c3(catenin delta-2 isoform 2) c4(106870) c5(106871, 106872, 106873, 106874, 106875) c6( ) c7(bM, T, asg, aYJ, aw, bO, Fb, Zh, nA, q, ad, eA, pN, bK, he, ae, af, aa, d, r, g, t, bz, fq, hq, eM, Be, m, it, iu); #c1(CTNND2) c2(NP_001275645) c3(catenin delta-2 isoform 3) c4(106876) c5(106877, 106878, 106879, 106880, 106881, 106882) c6( ) c7(bM, T, asg, aYJ, aw, bO, Fb, Zh, nA, q, ad, eA, pN, bK, he, ae, af, aa, d, r, g, t, bz, fq, hq, eM, Be, m, it, iu); #c1(CTNND2) c2(NP_001275646) c3 (catenin delta-2 isoform 4) c4( ) c5(106883, 106884, 106885, 106886, 106887) c6( ) c7(bM, T, asg, aYJ, aw, bO, Fb, Zh, nA, q, ad, eA, pN, bK, he, ae, af, aa, d, r, g, t, bz, fq, hq, eM, Be, m, it, iu); #c1(CTNND2) c2(NP_001323) c3(catenin delta-2 isoform 1) c4(116908, 116909, 116910, 116911) c5(116912, 116913, 116914, 116915, 116916, 116917, 116918, 116919, 116920, 116921, 116922, 116923, 116924, 116925, 116926, 116927, 116928, 116929, 116930, 116931) c6( ) c7(bM, T, asg, aYJ, aw, bO, Fb, Zh, nA, q, ad, eA, pN, bK, he, ae, af, aa, d, r, g, t, bz, fq, hq, eM, Be, m, it, iu); #c1(CTNS) c2(NP_001026851) c3(cystinosin isoform 1 precursor) c4(29645, 29646, 29647) c5(29648, 29649, 29650, 29651, 29652, 29653, 29654, 29655, 29656, 29657, 29658, 29659, 29660) c6( ) c7(IR, aYM, aAh, awd, dq, aj, Lq, dy, aYN, UE, bl, aYK, aYL); #c1(CTNS) c2(NP_004928) c3(cystinosin isoform 2 precursor) c4(150844, 150845) c5(150846, 150847, 150848, 150849, 150850, 150851, 150852, 150853, 150854, 150855, 150856, 150857, 150858, 150859, 150860, 150861, 150862, 150863, 150864, 150865, 150866, 150867, 150868, 150869, 150870, 150871, 150872, 150873, 150874, 150875, 150876, 150877, 150878, 150879, 150880, 150881, 150882, 150883, 150884, 150885, 150886, 150887, 150888, 150889, 150890, 150891, 150892, 150893, 150894, 150895, 150896, 150897, 150898, 150899, 150900, 150901, 150902, 150903, 150904, 150905, 150906, 150907, 150908, 150909, 150910, 150911, 150912, 150913, 150914, 150915, 150916, 150917, 150918, 150919, 150920, 150921, 150922, 150923, 150924, 150925, 150926, 150927, 150928, 150929, 150930, 150931, 150932, 150933, 150934, 150935, 150936, 150937, 150938, 150939, 150940, 150941, 150942, 150943, 150944, 150945, 150946, 150947, 150948, 150949, 150950, 150951, 150952, 150953, 150954, 150955, 150956, 150957, 150958, 150959, 150960, 150961, 150962, 150963, 150964, 150965, 150966, 150967, 150968, 150969, 150970, 150971, 150972, 150973, 150974, 150975, 150976, 150977, 150978, 150979, 150980, 150981, 150982, 150983, 150984, 150985, 150986, 150987, 150988, 150989, 150990, 150991, 150992, 150993, 150994, 150995, 150996, 150997, 150998, 150999, 151000, 151001, 151002, 151003, 151004, 151005) c6(151006, 151007) c7(IR, aYM, aAh, awd, dq, aj, Lq, dy, aYN, UE, bl, aYK, aYL); #c1(CTPS1) c2(NP_001288166) c3(CTP synthase 1 isoform b) c4( ) c5(113666, 113667, 113668, 113669, 113670, 113671) c6( ) c7(hF, nY); #c1(CTPS1) c2(XP_011539123) c3(CTP synthase 1 isoform X2) c4(337198, 337199) c5(337200, 337201) c6( ) c7(hF, nY); #c1(CTPS1) c2(XP_011539124) c3(CTP synthase 1 isoform X3) c4(337202) c5(337203, 337204, 337205, 337206, 337207, 337208) c6( ) c7(hF, nY); #c1(CTR9) c2(NP_055448) c3(RNA polymerase-associated protein CTR9 homolog) c4(181213) c5(181214, 181215, 181216, 181217, 181218, 18/219) c6( ) c7(pl, j, m, bA, ps); #c1(CTRB1) c2(NP_001897) c3(chymotrypsinogen B precursor) c4(122082) c5(122083, 122084, 122085, 122086, 122087, 122088) c6( ) c7(W, aj, J); #c1(CTRB2) c2(NP_001020371) c3(chymotrypsinogen B2 precursor) c4(28667) c5(28668, 28669, 28670, 28671, 28672, 28673, 28674, 28675) c6(28676, 28677) c7(ar, aC, A, W); #c1(CTRC) c2(XP_011538852) c3(chymotrypsin-C isoform X1) c4( ) c5(336921) c6( ) c7(ar, A, Gs, aYO, iG, alx, ayZ); #c1(CTRL) c2(NP_001898) c3(chymotrypsin-like protease CTRL-1 precursor) c4(122089) c5(122090, 122091, 122092, 122093, 122094, 122095, 122096, 122097, 122098, 122099) c6( ) c7(aW, Of, cr, al, Og, aEW, cd); #c1(CTSA) c2(NP_000299) c3(lysosomal protective protein isoform a precursor) c4(6714) c5(6715, 6716, 6717, 6718, 6719, 6720, 6721, 6722, 6723) c6( ) c7(T, aYP, aYa, zt, aYQ, aDj, cz, Lp, h, aYR, Lq, bt, dr); #c1(CTSA) c2(NP_001121167) c3(lysosomal protective protein isoform b precursor) c4(45297, 45298) c5(45299, 45300, 45301, 45302, 45303, 45304, 45305, 45306, 45307, 45308, 45309) c6( ) c7(T, aYP, aYa, zt, aYQ, aDj, cz, Lp, h, aYR, Lq, bt, dr); #c1(CTSA) c2(NP_001161066) c3(lysosomal protective protein isoform c precursor) c4(65243, 65244) c5(65245, 65246, 65247, 65248, 65249, 65250, 65251, 65252, 65253, 65254, 65255, 65256, 65257) c6( ) c7(T, aYP, aYa, zt, aYQ, aDj, cz, Lp, h, aYR, Lq, bt, dr); #c1(CTSB) c2(NP_680093) c3(cathepsin B preproprotein) c4(242194, 242195, 242196, 242197) c5(242198, 242199, 242200, 242201, 242202, 242203, 242204, 242205, 242206, 242207, 242208, 242209, 242210, 242211, 242212, 242213, 242214, 242215, 242216, 242217, 242218, 242219, 242220, 242221, 242222, 242223, 242224, 242225, 242226, 242227, 242228, 242229, 242230, 242231, 242232, 242233, 242234, 242235, 242236, 242237, 242238, 242239, 242240, 242241, 242242, 242243, 242244, 242245, 242246, 242247, 242248, 242249, 242250, 242251, 242252, 242253, 242254, 242255, 242256, 242257, 242258, 242259, 242260, 242261, 242262) c6(242263, 242264, 242265, 242266, 242267, 242268, 242269, 242270, 242271, 242272, 242273, 242274, 242275, 242276, 242277, 242278, 242279, 242280, 242281, 242282) c7(oO, T, os, aC, pf, ea, e, bB, ci, aR, ip, dV, K, wt, H, cp, aYX, ajT, JI, aW, fc, uY, g, pl, eE, eF, cm, ba, eM, nB, mx, U, wB, aYS, m, aYV, lS, hh, at], dD, aw, aj, fr, cd, zo, ai, b, h, lG, yZ, dL, wb, bl, aXT, Dg, asp, MD, EU, aq, aXU, ajz, kk, adz, vU, ar, dB, aYU, hL, hR, Lq, B, ek, L, bL, aA, hz, C, aYW, Qo, yU, q, J, eA, ady, mj, yg, HC, aT, cy, aYT, bq, ae, af, eu, jf, bf, iU, oV, Y, axW, fO, bg, r, afq, lo, tu, en, cl, t, rY, wg, nM, aki, mf, aDj, ayZ, ao, bH, aZ, aat, oZ, eq, ad, av, hd, Db, yt, sO, oj, ab, Dj, sj, d, yx, eH, ff, tv, Ej, aF, hp, bz, Hw, DT, bE, i, y, agT, us); #c1(CTSC) c2(NP_001107645) c3(dipeptidyl peptidase 1 isoform c precursor) c4(42939) c5(42940, 42941, 42942, 42943, 42944, 42945) c6( ) c7(dD, aYZ, ea, e, ht, by, aA, cz, v, j, eq, Tz, aZa, aji, qG, dL, lQ, bq, eL, aYY, eu, MD, axW, yx, bg, hl, fJ, ajl, ba, uz); #c1(CTSC) c2(NP_001805) c3 (dipeptidyl peptidase 1 isoform a preproprotein) c4(121265, 121266) c5(121267, 121268, 121269, 121270, 121271, 121272, 121273, 121274, 121275, 121276, 121277, 121278) c6( ) c7(dD, aYZ, ea, e, ht, by, aA, cz, v, j, eq, Tz, aZa, aji, qG, dL, lQ, bq, eL, aYY, eu, MD, axW, yx, bg, hl, fJ, ajl, ba, uz); #c1(CTSC) c2(NP_680475) c3(dipeptidyl peptidase 1 isoform b precursor) c4(242291) c5(242292, 242293, 242294, 242295, 242296, 242297) c6( ) c7(dD, aYZ, ea, e, ht, by, aA, cz, v, j, eq, Tz, aZa, aji, qG, dL, lQ, bq, eL, aYY, eu, MD, axW, yx, bg, hl, fJ, ajl, ba, uz); #c1 (CTSD) c2(NP_001900) c3(cathepsin D preproprotein) c4(122100) c5(122101, 122102, 122103, 122104, 122105, 122106) c6( ) c7(bM, T, dt, aC, pf, aw, ha, KW, qy, n, j, ci, fr, PJ, il, q, ad, asS, aao, eA, iu, aaF, b, aE, cc, yt, aAh, awd, qK, aT, DO, aZd, dL, K, acI, an, ae, af, eL, aa, ak, d, aW, Y, CM, bg, ff, LZ, r, aq, en, cl, g, t, QK, bz, as, cm, rY, dS, bN, ba, tj, jV, aZc, bE, AM, U, hL, uz, wR, dy, m, aPM, aZb, y, B); #c1(CTSE) c2(NP_001901) c3(cathepsin E isoform a preproprotein) c4(122107) c5(122108, 122109, (22110, 122/11, 122112, 122113) c6( ) c7(YL, gx, T, aE, s, aA, aq, dL, kp, ci, aF, eE, LV, eL); #c1(CTSE) c2(NP_683865) c3(cathepsin E isoform b preproprotein) c4(242590, 242591) c5(242592, 242593, 242594, 242595, 242596, 242597) c6( ) c7(YL, gx, T, aE, s, aA, aq, dl, kp, ci, aF, eE, LV, eL); #c1(CTSE) c2(XP_011507546) c3(cathepsin E isoform X1) c4(297470) c5(297471, 297472, 297473, 297474, 297475, 297476) c6( ) c7(YL, gx, T, aE, s, aA, aq, dL, kp, ci, af, eE, LV, eL); #c1(CTSF) c2(NP_003784) c3(cathepsin F precursor) c4(140783) c5(140784, 140785, 140786, 140787, 140788, 140789) c6( ) c7(vy, aZe, bK, aPT, rY); #c1(CTSG) c2(NP_001902) c3(cathepsin G preproprotein) c4(122114, 122115) c5(122116, 122117, 122118, 122119, 122120, 122121, 122122) c6( ) c7(bM, T, eR, e, aw, oA, C, hF, ab, h, hn, K, jj, aa, aZf, pb, ek, ck, Ta, wg, ac, fP, cj, uz, wR, dx, fD, ei, bt, kS); #c1(CTSG) c2(XP_011534801) c3(cathepsin G isoform X1) c4(331300, 331301, 331302, 331303, 331304, 331305, 331306, 331307, 331308, 331309, 331310, 331311) c5( ) c6( ) c7(bM, T, eR, e, aw, oA, C, hF, ab, h, hn, K, jj, aa, aZf, pb, ek, ck, Ta, wg, ac, fP, cj, uz, wR, dX, fD, ei, bt, kS); #c1 (CTSH) c2(NP_004381) c3(pro-cathepsin H preproprotein) c4(145956, 145957, 145958) c5(145959, 145960, 145961, 145962, 145963, 145964, 145965, 145966, 145967, 145968, 145969, 145970) c6( ) c7(bL, av, T, A, wB, Be, aT, at, aq, cl, dL, bk, hp, bm, sz, bt, B, aa); #c1(CTSH) c2(XP_011519578) c3 (pro-cathepsin H isoform X2) c4( ) c5(314241, 314242, 314243, 314244, 314245, 314246) c6( ) c7(bL, av, T, A, wB, Be, aT, at, aq, c, dL, bk, hp, bm, sz, bt, B, aa); #c1(CTSL) c2(XP_005251773) c3(cathepsin L1 isoform X1) c4(271150, 271151) c5(271152, 271153, 271154, 271155, 271156, 271157, 271158, 271159, 271160, 271161, 271162, 271163, 271164, 271165) c6(271166, 271167, 271168, 271169) c7(bL, T, dD, dA, ea, if, yG, ht, aA, n, OR, aj, ah, ci, v, hz, eq, qN, MT, q, J, zo, asS, eA, ip, av, hd, hw, db, rl, aT, h, bl, K, bq, hu, wt, fb, af, eu, sj, CL, A, d, aYX, iU, aW, Y, AQ, eH, ff, cr, r, dq, lo, aZg, EZ, en, bu, eE, t, cM, xe, eF, cm, ck, es, ar, Bv, nM, kP, eM, dB, mx, hr, hR, mf, dy, m, y, B); #c1(CTSL) c2(XP_011516565) c3(cathepsin L1 isoform X2) c4(309996) c5(309997, 309998, 309999, 310000, 310001, 310002, 310003, 310004, 310005, 310006, 310007, 310008, 310009, 310010, 310011) c6(310012, 310013, 310014) c7(bL, T, dD, dA, ea, if, yG, ht, aA, n, OR, aj, ah, ci, v, hz, eq, qN, MT, q, J, zo, asS, eA, ip, av, hd, hw, db, rl, aT, h, bl, K, bq, hu, wt, fb, af, eu, sj, CL, A, d, aYX, iU, aW, Y, AQ, eH, ff, cr, r, dq, lo, alg, EZ, en, bu, eE, t, cM, xe, eF, cm, ck, es, ar, Bv, nM, kP, eM, dB, mx, hr, hR, mf, dy, m, y, B); #c1(CTSO) c2(NP_001325) c3(cathepsin 0 preproprotein) c4(116932) c5(116933, 116934, 116935, 116936, 116937, 116938) c6( ) c7(aW, al); #c1(CTSV) c2(NP_001188504) c3(cathepsin L2 preproprotein) c4(79231) c5(79232, 79233, 79234, 79235, 79236, 79237, 79238, 79239, 79240, 79241) c6( ) c7(hM, A, cr, ab, eE, pb); #c1(CTSW) c2(NP_001326) c3(cathepsin W preproprotein) c4(116939) c5(116940, 116941, 116942, 116943, 116944, 116945) c6( ) c7(anY, cE); #c1(CTSZ) c2(NP_001327) c3(cathepsin Z preproprotein) c4(116946) c5(116947, 116948, 116949, 116950, 116951, 116952) c6( ) c7(ae, T, aE, ig, zt, aoo, az, cE, bt, aA, kp, aF, ci, g, aCK, H, tP, V, ad, L); #c1(CTTN) c2(NP_001171669) c3(src substrate cortactin isoform c) c4(71250, 71251) c5(71252, 71253, 71254, 71255, 71256, 71257, 71258, 71259, 71260, 71261, 71262, 71263) c6( ) c7(bM, T, FR, ht, aA, bB, n, BF, ci, cd, q, aE, aT, By, dL, K, hu, eL, aW, Y, Ls, az, ev, ff, r, aq, lo, aF, BG, en, g, iR, t, iK, ac, bE, m, rA, it, B); #c1(CTTN) c2(NP_005222) c3(src substrate cortactin isoform a) c4(154187) c5(154188, 154189, 154190, 154191, 154192, 154193) c6( ) c7(bM, T, fR, ht, aA, bB, n, BF, ci, cd, q, aE, aT, By, dL, K, hu, eL, aW, Y, Ls, az, ev, ff, r, aq, lo, aF, BG, en, g, iR, t, ik, ac, bE, m, rA, it, B); #c1(CTTN) c2(NP_612632) c3(src substrate cortactin isoform b) c4(236711) c5(236712, 236713, 236714, 236715, 236716, 236717) c6( ) c7(bM, T, fR, ht, aA, bB, n, BF, ci, cd, q, aE, aT, By, dL, K, hu, eL, aW, Y, Ls, az, ev, ff, r, aq, lo, aF, BG, en, g, iR, t, ik, ac, bE, m, rA, it, B); #c1(CTTNBP2) c2(NP_219499) c3(cortactin-binding protein 2) c4(228644) c5(228645, 228646, 228647, 228648, 228649, 228650, 228651, 228652) c6( ) c7(eW, aC, xH, j, jN); #c1(CTU1) c2(NP_660275) c3(cytoplasmic tRNA 2-thiolation protein 1) c4(240592) c5(240593, 240594, 240595, 240596, 240597, 240598, 240599) c6( ) c7(MO, aW, avp, bu); #c1(CTXN3) c2(NP_001041717) c3(cortexin-3) c4(34331) c5(34332, 34333, 34334, 34335, 34336, 34337, 34338, 34339, 34340, 34341) c6( ) c7(EQ); #c1(CUBN) c2(NP_001072) c3(cubilin precursor) c4(35565, 35566, 35567) c5( ) c6( ) c7(eR, oe, e, cz, mN, cQ, aj, j, aY, J, ad, af, xa, sj, A, aZh, Y, r, aZi, bz, apM, acC, c, UP, lp, mz, my); #c1(CUBN) c2(XP_011518010) c3(cubilin isoform X1) c4( ) c5(312023) c6( ) c7(eR, oe, e, cz, mN, cQ, aj, j, aY, J, ad, af, xa, sj, A, aZh, Y, r, aZi, bz, apM, acC, c, UP, lp, mz, my); #c1(CUBN) c2(XP_011518013) c3(cubilin isoform X4) c4(312024, 312025) c5(312026, 312027, 312028, 312029, 312030, 312031, 312032) c6( ) c7(eR, oe, e, cz, mN, cd, aj, j, aY, J, ad, af, xa, sj, A, aZh, Y, r, aZi, bz, apM, acC, c, UP, lp, mz, my); #c1(CUEDC1) c2(NP_001278954) c3(CUE domain-containing protein 1) c4(111088) c5(111089, 111090, 111091, 111092, 111093, 111094, 111095, 111096, 111097, 111098) c6( ) c7(V); #c1(CUEDC2) c2(NP_076945) c3(CUE domain-containing protein 2) c4(214329) c5(214330, 214331, 214332, 214333, 214334, 214335) c6( ) c7(aW, iU, aT); #c1(CUL1) c2(XP_011514934) c3(cullin-1 isoform X1) c4(307688, 307689, 307690) c5(307691, 307692, 307693, 307694, 307695, 307696, 307697, 307698, 307699, 307700, 307701, 307702, 307703, 307704, 307705, 307706, 307707, 307708, 307709, 307710, 307711, 307712) c6( ) c7(T, Kv, aW, Y, aT, cm, h, hP, t, B, y); #c1(CUL2) c2(NP_001185707) c3(cullin-2 isoform a) c4(76083) c5(76084, 76085, 76086, 76087, 76088, 76089, 76090, 76091, 76092) c6( ) c7(T, py, bP, iA, aW, go, ht, an, ff, fe, fr, en, hu, g, VB); #c1(CUL2) c2(NP_001185708) c3(cullin-2 isoform b) c4(76093) c5(76094, 76095) c6( ) c7(T, py, bP, iA, aW, go, ht, an, ff, fe, fr, en, hu, g, VB); #c1(CUL2) c2(XP_011518047) c3(cullin-2 isoform X2) c4( ) c5(312055) c6( ) c7(T, py, bP, iA, aW, go, ht, an, ff, fe, fr, en, hu, g, VB); #c1(CUL2) c2(XP_011518049) c3(cullin-2 isoform X1) c4(312056, 312057, 312058) c5(312059, 312060, 312061, 312062, 312063, 312064, 312065, 312066, 312067, 312068, 312069, 312070, 312071, 312072, 312073, 312074, 312075, 312076, 312077, 312078, 312079, 312080, 312081, 312082, 312083, 312084, 312085, 312086, 312087, 312088) c6( ) c7(T, py, bP, iA, aW, go, ht, an, ff, fe, fr, en, hu, g, VB); #c1(CUL3) c2(NP_001244126) c3(cullin-3 isoform 2) c4(91958) c5(91959, 91960, 91961, 91962, 91963, 91964) c6( ) c7(T, Lu, xX, d, bE, aW, Y, eL, aT, cz, bB, aq, dL, at, K, rP, bz, B, aZj); #c1(CUL3) c2(NP_001244127) c3(cullin-3 isoform 3) c4( ) c5(91965, 91966, 91967, 91968, 91969, 91970, 91971, 91972, 91973) c6( ) c7(T, Lu, xX, d, bE, aW, Y, eL, aT, cz, bB, aq, dL, at, K, rP, bz, B, aZj); #c1(CUL3) c2(NP_003581) c3(cullin-3 isoform 1) c4(139373, 139374) c5(139375, 139376, 139377, 139378, 139379, 139380, 139381) c6( ) c7(T, Lu, xX, d, bE, aW, Y, eL, aT, cz, bB, aq, dl, at, K, rP, bz, B, aZj); #c1 (CUL4A) c2(NP_001008895) c3(cullin-4A isoform 1) c4(23472, 23473) c5(23474, 23475, 23476, 23477, 23478, 23479, 23480, 23481, 23482) c6( ) c7(ae, T, av, ib, yo, s, nO, aW, aT, hR, aq, h, K, m, t, bz, B, ad); #c1(CUL4A) c2(XP_011535825) c3(cullin-4A isoform X1) c4( ) c5(333027, 333028, 333029, 333030, 333031, 333032, 333033, 333034, 333035, 333036, 333037) c6( ) c7(ae, T, av, ib, yo, s, nO, aW, aT, hR, aq, h, K, m, t, bz, B, ad); #c1(CUL4B) c2(NP_001073341) c3(cullin-48 isoform 2) c4(35966) c5(35967, 35968, 35969, 35970, 35971, 35972) c6( ) c7(alT, T, aZk, ha, nf, ey, nA, bX, aU); #c1(CUL4B) c2(NP_003579) c3(cullin-48 isoform 1) c4(139366) c5(139367, 139368, 139369, 139370, 139371, 139372) c6( ) c7(alT, T, aZk, ha, nf, ey, nA, bX, aZl); #c1(CUL4B) c2(XP_006724848) c3(cullin-48 isoform X6) c4(296464) c5(296465, 296466, 296467, 296468, 296469, 296470) c6( ) c7(alT, T, aZk, ha, nf, ey, nA, bX, aU); #c1(CUL4B) c2(XP_011529702) c3(cullin-48 isoform X4) c4(325327) c5(325328, 325329, 325330, 325331, 325332, 325333) c6( ) c7(alT, T, aZk, ha, nf, ey, nA, bX, aU); #c1(CUL5) c2(NP_003469) c3(cullin-5) c4(138589) c5(138590, 138591, 138592, 138593, 138594, 138595, 138596, 138597, 138598, 138599) c6( ) c7(T, aW, if, aCH, gN, cj, aT, VT, h, qN, B); #c1(CUL7) c2(NP_001161842) c3(cullin-7 isoform 1) c4(66143) c5(66144, 66145, 66146, 66147, 66148, 66149) c6( ) c7(eM, aq, aCm, fr, aCo, ei, cm, nJ, rv, B); #c1 (CUL7) c2(NP_055595) c3(cullin-7 isoform 2) c4(182159) c5( ) c6( ) c7(eM, aq, aCm, fr, aCo, ei, cm, nJ, rv, B); #c1 (CUL7) c2(XP_005249560) c3(cullin-7 isoform X3) c4( ) c5(269705, 269706, 269707, 269708, 269709, 269710) c6( ) c7(eM, aq, aCm, fr, aCo, ei, cm, nJ, rv, B); #c1(CUL9) c2(NP_055904) c3(cullin-9) c4(184374) c5(184375, 184376, 184377, 184378, 184379, 184380) c6( ) c7(ac, aCl, an, cm, bA, hX); #c1(CUL9) c2(XP_011512727) c3(cullin-9 isoform X3) c4(303760) c5(303761, 303762, 303763, 303764, 303765, 303766) c6( ) c7(ac, aCl, an, cm, bA, hX); #c1(CUL9) c2(XP_011512731) c3(cullin-9 isoform X7) c4( ) c5(303767) c6( ) c7(ac, aCl, an, cm, bA, hX); #c1(CUTA) c2(NP_001014433) c3(protein CutA isoform 1) c4(25862, 25863) c5(25864, 25865, 25866, 25867, 25868, 25869, 25870, 25871) c6( ) c7(eZ); #c1(CUTA) c2(NP_001014837) c3(protein CutA isoform 2 precursor) c4( ) c5(26051, 26052, 26053, 26054, 26055, 26056, 26057, 26058) c6( ) c7(eZ); #c1(CUTA) c2(NP_001014840) c3(protein CutA isoform 3 precursor) c4(26059, 26060) c5(26061, 26062, 26063, 26064, 26065, 26066, 26067, 26068, 26069, 26070, 26071, 26072, 26073, 26074, 26075, 26076) c6( ) c7(eZ); #c1(CUX1) c2(NP_001189472) c3(protein CASP isoform d) c4(80189) c5(80190, 80191, 80192, 80193, 80194, 80195) c6( ) c7(bM, T, bO, fd, bB, ci, aR, amW, aE, K, zy, H, eG, LC, aW, Ls, eE, cm, eM, avS, iT, vA, wB, U, PV, Hb, m, cZ, wu, aw, aj, fr, qN, ec, KN, KH, dd, Yd, fe, op, Fi, ahC, it, L, aA, V, q, eA, WY, aql, aT, dz, adm, jj, rz, ae, af, jf, oV, Y, r, wA, t, xw, iK, bN, ck, iM, ac, c, rA, bt, hS, jh, hF, ad, av, Tg, aCJ, ji, wm, fb, WZ, je, afi, af, bz, LB); #c1(CUX1) c2(NP_001189473) c3(protein CASP isoform e) c4(80196) c5(80197, 80198, 80199, 80200, 80201, 80202) c6( ) c7(bM, T, bO, fd, bB, ci, aR, amW, aE, K, zy, H, eG, LC, aW, Ls, eE, cm, eM, avS, iT, vA, wB, U, PV, Hb, m, cZ, wu, aw, aj, fr, qN, ec, KN, kH, dd, Yd, fe, op, Fi, ahC, it, L, aA, V, q, eA, WY, aql, aT, dz, adm, jj, rz, ae, af, jf, oV, Y, r, wA, t, xw, ik, bN, ck, iM, ac, c, rA, bt, hS, jh, hF, ad, av, Tg, aCJ, ji, wm, fb, WZ, je, afi, aF, bz, LB); #c1(CUX1) c2(NP_001189474) c3(protein CASP isoform f) c4(80203) c5(80204, 80205, 80206, 80207, 80208, 80209) c6( ) c7(bM, T, bO, fd, bB, ci, aR, amW, aE, K, zy, H, eG, LC, aW, Ls, eE, cm, eM, avS, iT, vA, wB, U, PV, Hb, m, cZ, wu, aw, aj, fr, qN, ec, KN, KH, dd, Yd, fe, op, Fi, ahC, it, L, aA, V, q, eA, WY, aql, aT, dz, adm, jj, rz, ae, af, jf, oV, Y, r, wA, t, xw, iK, bN, ck, iM, ac, c, rA, bt, hS, jh, hF, ad, av, Tg, aCJ, ji, wm, fb, WZ, je, afi, aF, bz, LB); #c1(CUX1) c2(NP_001189475) c3(protein CASP isoform g) c4(80210) c5(80211, 80212, 80213, 80214, 80215, 80216) c6( ) c7(bM, T, bO, fd, bB, ci, aR, amW, aE, K, zy, H, eG, LC, aW, Ls, eE, cm, eM, avS, iT, vA, wB, U, PV, Hb, m, cZ, wu, aw, aj, fr, qN, ec, KN, kH, dd, Yd, fe, op, Fi, ahC, it, L, aA, V, q, eA, WY, aql, aT, dz, adm, j, rz, ae, af, jf, oV, Y, r, wA, t, xw, ik, bN, ck, iM, ac, c, rA, bt, hS, jh, hF, ad, av, Tg, aCJ, ji, wm, fb, WZ, je, afi, aF, bz, LB); #c1(CUX1) c2(NP_001904) c3(protein CASP isoform b) c4(122123, 122124, 122125) c5(122126, 122127, 122128, 122129, 122130, 122131, 122132) c6( ) c7(bM, T, bO, fd, bB, ci, aR, amW, aE, K, zy, H, eG, LC, aW, Ls, eE, cm, eM, avS, iT, vA, wB, U, PV, Hb, m, cZ, wu, aw, aj, fr, qN, ec, KN, kH, dd, Yd, fe, op, Fi, ahC, it, L, aA, V, q, eA, WY, aql, aT, dz, adm, jj, rz, ae, af, jf, oV, Y, r, wA, t, xw, ik, bN, ck, iM, ac, c, rA, bt, hS, jh, hF, ad, av, Tg, aCJ, ji, wm, fb, WZ, je, afi, aF, bz, LB); #c1(CUX1) c2(NP_852477) c3(protein CASP isoform c) c4(255012) c5(255013, 255014, 255015, 255016, 255017, 255018) c6( ) c7(bM, T, bO, fd, bB, ci, aR, amW, aE, K, zy, H, eG, LC, aW, Ls, eE, cm, eM, avS, iT, vA, wB, U, PV, Hb, m, cZ, wu, aw, aj, fr, qN, ec, KN, KH, dd, Yd, fe, op, Fi, ahC, it, L, aA, V, q, eA, WY, aql, aT, dz, adm, jj, rz, ae, af, jf, oV, Y, r, wA, t, xw, iK, bN, ck, iM, ac, c, rA, bt, hS, jh, hF, ad, av, Tg, aCJ, ji, wm, fb, WZ, je, afi, af, bz, LB); #c1(CUX1) c2(NP_853530) c3(protein CASP isoform a) c4(255149) c5(255150, 255151, 255152, 255153, 255154) c6( ) c7(bM, T, bO, fd, bB, ci, aR, amW, aE, K, zy, H, eG, LC, aW, Ls, eE, cm, eM, avS, iT, vA, wB, U, PV, Hb, m, cZ, wu, aw, aj, fr, qN, ec, KN, KH, dd, Yd, fe, op, Fi, ahC, it, L, aA, V, q, eA, WY, aql, aT, dz, adm, jj, rz, ae, af, jf, OV, Y, r, wA, t, xw, iK, bN, ck, iM, ac, c, rA, bt, hS, jh, hF, ad, av, Tg, aCJ, ji, wm, fb, WZ, je, afi, af, bz, LB); #c1(CUX1) c2(XP_005250207) c3(protein CASP isoform X1) c4( ) c5(270200) c6( ) c7(bM, T, bO, fd, bB, ci, aR, amW, aE, K, zy, H, eG, LC, aW, Ls, eE, cm, eM, avS, iT, vA, wB, U, PV, Hb, m, cZ, wu, aw, aj, fr, qN, ec, KN, kH, dd, Yd, fe, op, Fi, ahC, it, L, aA, V, q, eA, WY, aql, aT, dz, adm, jj, rz, ae, af, jf, oV, Y, r, wA, t, xw, iK, bN, ck, iM, ac, c, rA, bt, hS, jh, hF, ad, av, Tg, aCJ, ji, wm, fb, WZ, je, afi, aF, bz, LB); #c1(CUX1) c2(XP_005250211) c3(protein CASP isoform X7) c4( ) c5(270201, 270202, 270203, 270204, 270205, 270206, 270207, 270208, 270209, 270210, 270211, 270212) c6( ) c7(bM, T, bO, fd, bB, ci, aR, amW, aE, K, zy, H, eG, LC, aW, Ls, eE, cm, eM, avS, iT, vA, wB, U, PV, Hb, m, cZ, wu, aw, aj, fr, qN, ec, KN, kH, dd, Yd, fe, op, Fi, ahC, it, L, aA, V, q, eA, WY, aql, aT, dz, adm, jj, rz, ae, af, jf, oV, Y, r, wA, t, xw, iK, bN, ck, iM, ac, c, rA, bt, hS, jh, hF, ad, av, Tg, aCJ, ji, wm, fb, WZ, je, afi, aF, bz, LB); #c1(CUX1) c2(XP_006715917) c3(protein CASP isoform X3) c4(289938, 289939) c5(289940, 289941, 289942, 289943, 289944, 289945, 289946, 289947, 289948, 289949, 289950, 289951) c6( ) c7(bM, T, bO, fd, bB, ci, aR, amW, aE, K, zy, H, eG, LC, aW, Ls, eE, cm, eM, avS, iT, vA, wB, U, PV, Hb, m, cZ, wu, aw, aj, fr, qN, ec, KN, KH, dd, Yd, fe, op, Fi, ahC, it, L, aA, V, q, eA, WY, aql, aT, dz, adm, jj, rz, ae, af, jf, oV, Y, r, wA, t, xw, ik, bN, ck, iM, ac, c, rA, bt, hS, jh, hF, ad, av, Tg, aCJ, ji, wm, fb, WZ, je, afi, aF, bz, LB); #c1 (CUX1) c2(XP_011514125) c3(protein CASP isoform X4) c4(306721, 306722) c5(306723, 306724, 306725, 306726, 306727, 306728, 306729, 306730, 306731, 306732, 306733, 306734) c6( ) c7(bM, T, bO, fd, bB, ci, aR, amW, aE, K, zy, H, eG, LC, aW, Ls, eE, cm, eM, avS, iT, vA, wB, U, PV, Hb, m, cZ, wu, aw, aj, fr, qN, ec, KN, KH, dd, Yd, fe, op, Fi, ahC, it, L, aA, V, q, eA, WY, aql, aT, dz, adm, jj, rz, ae, af, jf, oV, Y, r, wA, t, xw, ik, bN, ck, iM, ac, c, rA, bt, hS, jh, hF, ad, av, Tg, aCJ, ji, wm, fb, WZ, je, afi, af, bz, LB); #c1(CUX1) c2(XP_011514127) c3 (protein CASP isoform X6) c4( ) c5(306735) c6( ) c7(bM, T, bO, fd, bB, ci, aR, amW, aE, K, zy, H, eG, LC, aW, Ls, eE, cm, eM, avS, lT, vA, wB, U, PV, Hb, m, cZ, wu, aw, aj, fr, qN, ec, KN, KH, dd, Yd, fe, op, Fi, ahC, it, L, aA, V, q, eA, WY, aql, aT, dz, adm, jj, rz, ae, af, jf, oV, Y, r, wA, t, xw, iK, bN, ck, iM, ac, c, rA, bt, hS, jh, hF, ad, av, Tg, aCJ, ji, wm, fb, WZ, je, afi, af, bz, LB); #c1(CUX2) c2(NP_056082) c3(homeobox protein cut-like 2) c4(185581) c5(185582, 185583, 185584, 185585, 185586, 185587, 185588) c6( ) c7(mU, A, mW, mZ, mS, mY, bK, mV, mT, bN, mX); #c1(CUX2) c2(XP_011536363) c3(homeobox protein cut-like 2 isoform X1) c4( ) c5(333685) c6( ) c7(mU, A, mW, mZ, mS, mY, bK, mV, mT, bN, mX); #c1(CUX2) c2(XP_011536374) c3(homeobox protein cut-like 2 isoform X5) c4(333686) c5(333687, 333688, 333689, 333690, 333691) c6( ) c7(mU, A, mW, mZ, mS, mY, bK, mV, mT, bN, mX); #c1(CUZD1) c2(NP_071317) c3(CUB and zona pellucida-like domain-containing protein 1 precursor) c4(210896, 210897, 210898) c5(210899, 210900, 210901, 210902, 210903, 210904, 210905, 210906, 210907, 210908, 210909, 210910, 210911, 210912, 210913, 210914, 210915, 210916, 210917, 210918, 210919, 210920, 210921, 210922, 210923, 210924, 210925, 210926, 210927, 210928, 210929, 210930, 210931, 210932) c6( ) c7(fe, t, T, aE, m); #c1(CWC22) c2(NP_065994) c3(pre-mRNA-splicing factor CWC22 homolog) c4(206627, 206628, 206629) c5(206630, 206631, 206632, 206633, 206634, 206635, 206636, 206637, 206638, 206639, 206640, 206641, 206642, 206643, 206644, 206645, 206646, 206647) c6( ) c7(bM, ac, pN, bE, aw, eZ, bB, v, ad); #c1(CWC27) c2(NP_001284573) c3(peptidyl-prolyl cis-trans isomerase CWC27 homolog isoform 2) c4(112062) c5(112063, 112064, 112065, 112066, 112067, 112068) c6( ) c7(j); #c1(CWC27) c2(NP_005860) c3(peptidyl-prolyl cis-trans isomerase CWC27 homolog isoform 1) c4(159997) c5(159998, 159999, 160000, 160001, 160002, 160003) c6( ) c7(j); #c1(CWF19L1) c2(NP_001290333) c3(CWF19-like protein 1 isoform 2) c4(115199) c5(115200, 115201, 115202, 115203, 115204, 115205) c6( ) c7(fc, cP); #c1(CWF19LI) c2(NP_060764) c3(CWF19-like protein 1 isoform 1) c4(197678) c5(197679, 197680, 197681, 197682, 197683, 197684, 197685, 197686, 197687, 197688) c6( ) c7(fc, cP); #c1(CWF19L2) c2(NP_689647) c3(CWF19-like protein 2) c4(243425, 243426, 243427) c5(243428, 243429, 243430, 243431, 243432, 243433, 243434, 243435, 243436, 243437, 243438, 243439) c6( ) c7(cF, cz, aT); #c1(CWF19L2) c2(XP_006718831) c3(CWF19-like protein 2 isoform X2) c4( ) c5(292018, 292019, 292020, 292021, 292022, 292023) c6( ) c7(cF, cz, aT); #c1(CWH43) c2(NP_079363) c3(PGAP2-interacting protein isoform 1) c4(217820) c5(217821, 217822, 217823, 217824, 217825, 217826) c6( ) c7(F); #c1(CWH43) c2(XP_011512060) c3(PGAP2-interacting protein isoform X3) c4( ) c5(302700) c6( ) c7(F); #c1(CX3CLI) c2(NP_002987) c3(fractalkine isoform 1 precursor) c4(134197) c5(134198, 134199, 134200, 134201, 134202, 134203, 134204, 134205) c6( ) c7(bM, T, ea, aFV, aZn, GO, aa, UC, aW, mQ, hl, vs, eE, cm, nB, U, uo, nw, aZo, m, wx, eg, hh, dt, dD, aw, fN, qW, cd, ql, rl, h, SH, lG, wb, aDp, wC, ap, hQ, fe, kp, kT, W, JM, bm, va, eo, cB, bL, cz, v, j, V, eA, eJ, rS, aT, Oi, cy, bq, an, ahE, ae, af, fO, aZm, r, lo, ahl, LV, pb, hc, cS, EY, fD, CS, uG, jh, wV, eq, ad, dP, aZp, tT, jo, cr, hv, Fq, iV, ek, D, aS, ME, lC, y); #c1(CX3CRI) c2(NP_001164645) c3(CX3C chemokine receptor 1 isoform a) c4(67128, 67129, 67130) c5(67131, 67132, 67133, 67134, 67135, 67136, 67137, 67138, 67139, 67140, 67141, 67142, 67143, 67144, 67145, 67146, 67147) c6( ) c7(ID, bM, T, fR, ea, e, al, aY, aE, K, GO, bc, hm, UC, ly, li, aW, vs, g, as, eE, cm, uC, y, eM, NI, nw, wx, m, hh, de, dt, dD, My, aw, aj, qW, cd, eW, Mo, Hx, vC, rl, cU, SH, h, dL, ey, aDp, Um, wC, ZC, ap, kp, lx, KT, W, lf, JM, bm, hs, at, va, cB, bL, Dw, eR, cz, cR, v, j, V, eA, J, eJ, di, rS, aT, agD, Oi, cy, UN, bq, ae, af, als, fO, aZm, LV, sK, pb, wg, ac, iM, iQ, hc, cS, dy, EY, aZr, iW, uG, jh, vr, aZq, uu, eq, ad, aZp, F, ot, oz, tT, ul, DI, d, je, cr, dN, Fq, bz, nb, Om, dJ, aS, agl, ei, ME, lC, xg, hv); #c1(CX3CRI) c2(NP_001328) c3(CX3C chemokine receptor 1 isoform b) c4(116953, 116954) c5(116955, 116956, 116957, 116958, 116959, 116960, 116961, 116962, 116963, 116964, 116965, 116966, 116967, 116968, 116969, 116970, 116971, 116972, 116973, 116974, 116975) c6( ) c7(ID, bM, T, fR, ea, e, al, aY, aE, K, GO, bc, hm, UC, ly, li, aW, vs, g, as, BE, cm, uC, y, eM, NI, nw, wx, m, hh, de, dt, dD, My, aw, aj, qW, cd, eW, Mo, Hx, vC, rl, cU, SH, h, dL, ey, aDp, Um, wC, ZC, ap, kp, lx, kT, W, lf, JM, bm, hs, at, va, cB, bL, Dw, eR, cz, cR, v, j, V, eA, J, eJ, di, rS, aT, agD, Oi, cy, UN, bq, ae, af, als, fO, aZm, LV, sK, pb, wg, ac, iM, iQ, hc, cS, dy, EY, aZr, iW, uG, jh, vr, aZq, uu, eq, ad, aZp, F, ot, oz, tT, ul, DI, d, je, cr, dN, Fq, bz, nb, Om, dJ, aS, agl, ei, ME, lC, xg, hv); #c1(CXADR) c2(NP_001193993) c3(coxsackievirus and adenovirus receptor isoform 3 precursor) c4(83442) c5(83443, 83444, 83445, 83446, 83447, 83448) c6( ) c7(gV, T, dA, fR, qb, aw, jh, aA, bB, fd, wV, v, rf, ci, q, ad, eA, ip, eW, av, Yn, aT, dL, ae, af, eL, d, B, PP, s, hG, aW, Y, OX, oV, eH, ff, r, lo, en, g, eF, t, cm, bz, ik, ck, KT, bP, aDX, eM, aHn, dB, bE, mx, hL, cj, gZ, iS, MJ, m, gT, bt, X); #c1(CXADR) c2(NP_001193994) c3(coxsackievirus and adenovirus receptor isoform 4 precursor) c4(83449) c5(83450, 83451, 83452, 83453, 83454, 83455) c6( ) c7(gV, T, dA, fR, qb, aw, jh, aA, bB, fd, wV, v, rf, ci, q, ad, eA, ip, eW, av, Yn, aT, dL, ae, af, eL, d, B, PP, s, hG, aW, Y, OX, oV, eH, ff, r, lo, en, g, eF, t, cm, bz, ik, ck, kT, bP, aDX, eM, aHn, dB, bE, mx, hL, cj, gZ, iS, MJ, m, gT, bt, X); #c1(CXADR) c2(NP_001193995) c3(coxsackievirus and adenovirus receptor isoform 5 precursor) c4(83456) c5(83457, 83458, 83459, 83460, 83461, 83462) c6( ) c7(gV, T, dA, fR, qb, aw, jh, aA, bB, fd, wV, v, rf, ci, q, ad, eA, ip, eW, av, Yn, aT, dL, ae, af, eL, d, B, PP, s, hG, aW, Y, OX, oV, eH, ff, r, lo, en, g, eF, t, cm, bz, ik, ck, kT, bP, aDX, eM, aHn, dB, bE, mx, hL, cj, gZ, iS, MJ, m, gT, bt, X); #c1(CXADR) c2(NP_001329) c3(coxsackievirus and adenovirus receptor isoform 1 precursor) c4(116976, 116977) c5(116978, 116979, 116980, 116981, 116982, 116983, 116984, 116985, 116986, 116987, 116988, 116989) c6( ) c7(gV, T, dA, fR, qb, aw, jh, aA, bB, fd, wV, v, rf, ci, q, ad, eA, ip, eW, av, Yn, aT, dL, ae, af, eL, d, B, PP, s, hG, aW, Y, OX, oV, eH, ff, r, lo, en, g, eF, t, cm, bz, iK, ck, kT, bP, aDX, eM, aHn, dB, bE, mx, hL, cj, gZ, iS, MJ, m, gT, bt, X); #c1(CXADR) c2(XP_011527780) c3 (coxsackievirus and adenovirus receptor isoform X4) c4(322748) c5( ) c6( ) c7(gV, T, dA, fR, qb, aw, jh, aA, bB, fd, wV, v, rf, ci, q, ad, eA, ip, eW, av, Yn, aT, dL, ae, af, eL, d, B, PP, s, hG, aW, Y, OX, oV, eH, ff, r, lo, en, g, eF, t, cm, bz, ik, ck, KT, bP, aDX, eM, aHn, dB, bE, mx, hL, cj, gZ, iS, MJ, m, gT, bt, X); #c1(CXADR) c2(XP_011527781) c3(coxsackievirus and adenovirus receptor isoform X5) c4( ) c5(322749, 322750, 322751, 322752, 322753, 322754) c6( ) c7(gV, T, dA, fR, qb, aw, jh, aA, bB, fd, wV, v, rf, ci, q, ad, eA, ip, eW, av, Yn, aT, dL, ae, af, eL, d, B, PP, s, hG, aW, Y, OX, oV, eH, ff, r, lo, en, g, eF, t, cm, bz, ik, ck, kT, bP, aDX, eM, aHn, dB, bE, mx, hL, cj, gZ, iS, MJ, m, gT, bt, X); #c1(CXCL1) c2(NP_001502) c3(growth-regulated alpha protein precursor) c4(118246) c5(118247, 118248, 118249, 118250, 118251, 118252) c6( ) c7(aZt, T, dD, bM, dA, Dw, qb, aD, al, jx, aA, bB, j, ci, eq, bh, qN, V, q, Pm, ad, kL, dP, Ks, wD, hd, rw, qp, akJ, aT, cU, lG, Ds, dl, alr, lQ, NM, ae, le, BO, s, ff, aW, sp, Y, az, hl, oV, r, m, KD, lo, aF, yq, en, as, eE, t, iV, fq, ck, D, gk, lt, aZu, pc, bE, AM, Jp, wR, m, fD, hh, y, bt, B); #c1 (CXCL10) c2(NP_001556) c3(C-X-C motif chemokine 10 precursor) c4(118934) c5(118935, 118936, 118937, 118938, 118939, 118940) c6( ) c7(bM, T, FR, if, al, bO, fd, cL, ci, bh, aR, wa, azY, xy, dV, Ds, K, hu, wt, aWf, eL, bc, aa, amN, aW, qS, fc, hl, Ku, pl, eE, uC, ba, y, aZx, eM, aDs, dc, ki, cj, m, gn, hh, aDi, Ht, dD, afA, ht, aFc, fN, n, aj, fr, apt, aBl, uE, qN, Za, qW, ai, eh, eW, aZv, cU, lG, h, dL, eV, pi, cY, xP, Nr, A, fJ, aq, po, kp, CO, xe, hL, at, aDx, aEG, nz, B, ka, aZy, avm, dk, lH, yk, jx, eX, aA, fF, hz, v, j, V, eA, J, o, ady, jZ, aT, cy, aLM, aDt, XZ, eu, hM, s, Y, bg, r, lo, en, aaD, t, pb, ck, OW, ac, nM, ll, ig, iQ, hc, AH, wR, dy, bt, vi, Kd, aD, df, jh, auq, dG, eq, jE, wV, pz, ad, aoH, dP, hX, F, ds, hD, vB, ji, lQ, amY, NM, vP, yF, ul, d, ff, wc, aZw, cr, eZ, aF, Fq, iV, Nq, LB, D, gk, eU, Lj, dO, hE, kS, X, hv); #c1(CXCL11) c2(NP_005400) c3(C-X-C motif chemokine 11 isoform 1 precursor) c4(155718) c5(155719, 155720, 155721, 155722, 155723, 155724, 155725, 155726, 155727, 155728, 155729, 155730) c6(155731, 155732) c7(T, id, jx, jh, cl, j, ci, fr, eA, o, F, dV, aT, h, 1Q, db, eG, d, xR, aW, ff, Pg, dN, aq, lo, fe, un, en, as, aaQ, t, iR, aZz, xT, iQ, W, eM, ll, xO, dc, at, m, B); #c1(CXCL12) c2(NP_000600) c3(stromal cell-derived factor 1 isoform beta precursor) c4(13662) c5(13663, 13664, 13665, 13666, 13667, 13668, 13669) c6(13670) c7(ID, bM, T, aC, aiH, fR, ea, if, e, al, eb, bB, fd, axk, ci, bh, mF, ip, wa, x, bv, aE, il, rJ, K, eL, hm, ly, yo, aW, hl, g, as, eF, ir, cm, bw, iP, ba, ou, eM, iA, AM, iT, cj, k, Hb, m, ym, pJ, Vj, dD, sw, zt, iB, aw, NT, adk, n, aj, oY, fr, qN, qW, cd, ai, ke, JX, ft, jz, zw, ql, h, lG, eV, hn, dl, vx, A, apo, sp, fe, aq, Og, mo, zZ, cM, lx, aCz, dB, hL, bm, hR, pg, fL, aDE, aZD, cB, B, AN, aEC, it, bl, apE, aZA, dA, mr, fG, jx, eX, aA, tc, hz, j, C, uW, V, q, J, eA, mj, RU, RI, hb, afQ, ld, yg, aT, cy, bq, aFm, ae, af, eu, qw, Uf, s, nO, aZB, oV, Y, bg, r, en, cl, t, iK, pb, vk, ck, nM, ac, aZC, iQ, gX, cS, ij, zu, bt, Ze, aDj, VB, hS, kh, id, gN, jh, vr, dG, Gn, eq, tK, mC, ad, hX, dP, fU, av, F, py, yt, NS, io, hD, JK, yF, ul, d, lx, lR, hG, et, qt, ff, eH, Of, eZ, KD, aeV, aoF, aF, BG, iV, bz, ek, LB, es, D, Bv, arY, pc, Ew, bE, aED, ll, gZ, aS, ME, y, ra, X); #c1(CXCL12) c2(NP_001029058) c3(stromal cell-derived factor 1 isoform gamma precursor) c4(30754, 30755, 30756) c5( ) c6( ) c7(ID, bM, T, aC, aiH, fR, ea, if, e, al, eb, bB, fd, axk, ci, bh, mF, ip, wa, x, bv, aE, il, rJ, K, eL, hm, ly, yo, aW, hl, g, as, eF, ir, cm, bw, iP, ba, ou, eM, iA, AM, iT, cj, k, Hb, m, ym, pJ, Vj, dD, sw, zt, iB, aw, NT, adk, n, aj, oY, fr, qN, qW, cd, ai, ke, JX, ft, jz, zw, ql, h, lG, eV, hn, dl, vx, A, apo, sp, fe, aq, Og, mo, zZ, cM, lx, aCz, dB, hL, bm, hR, pg, fl, aDE, aZD, cB, B, AN, aEC, it, bL, apE, aZA, dA, mr, fG, jx, eX, aA, tc, hz, j, C, uW, V, q, J, eA, mj, RU, RI, hb, afQ, ld, yg, aT, cy, bq, aFm, ae, af, eu, qw, Uf, s, nO, aZB, oV, Y, bg, r, en, cl, t, iK, pb, vk, ck, nM, ac, aZC, iQ, gX, cS, ij, zu, bt, Ze, aDj, VB, hS, kh, id, gN, jh, vr, dG, Gn, eq, tK, mC, ad, hX, dP, fU, av, F, py, yt, NS, io, HD, JK, yF, ul, d, lx, lR, hG, et, qt, ff, eH, Of, eZ, kD, aeV, aoF, aF, BG, iV, bz, ek, LB, es, D, Bv, arY, pc, Ew, bE, aED, ll, gZ, aS, ME, y, ra, X); #c1(CXCL12) c2(NP_001171605) c3(stromal cell-derived factor 1 isoform delta precursor) c4(70739) c5(70740, 70741, 70742, 70743, 70744, 70745, 70746, 70747, 70748, 70749, 70750, 70751, 70752, 70753, 70754) c6(70755, 70756, 70757) c7(ID, bM, T, aC, aiH, fR, ea, if, e, al, eb, bB, fd, axk, ci, bh, mF, ip, wa, x, bv, aE, il, rJ, K, eL, hm, ly, yo, aW, hl, g, as, eF, ir, cm, bw, iP, ba, ou, eM, iA, AM, iT, cj, k, Hb, m, ym, pJ, Vj, dD, sw, zt, iB, aw, NT, adk, n, aj, oY, fr, qN, qW, cd, ai, ke, JX, ft, jz, zw, ql, h, lG, eV, hn, dl, vx, A, apo, sp, fe, aq, Og, mo, zZ, cM, lx, aCz, dB, hL, bm, hR, pg, fl, aDE, aZD, cB, B, AN, aEC, it, bL, apE, aZA, dA, mr, fG, jx, eX, aA, tc, hz, j, C, uW, V, q, J, eA, mj, RU, RI, hb, afQ, ld, yg, aT, cy, bq, aFm, ae, af, eu, qw, Uf, s, nO, aZB, oV, Y, bg, r, en, cl, t, iK, pb, vk, ck, nM, ac, aZC, iQ, gX, cS, ij, zu, bt, Ze, aDj, VB, hS, kh, id, gN, jh, vr, dG, Gn, eq, tK, mC, ad, hX, dP, fU, av, F, py, yt, NS, io, HD, JK, yF, uQ, d, lx, lR, hG, et, qt, ff, eH, Of, eZ, KD, aeV, aoF, aF, BG, iV, bz, eK, LB, es, D, Bv, arY, pc, Ew, bE, aED, ll, gZ, aS, ME, y, ra, X); #c1(CXCL12) c2(NP_954637) c3(stromal cell-derived factor 1 isoform alpha precursor) c4( ) c5(261110, 261111, 261112, 261113, 261114, 261115, 261116, 261117, 261118, 261119, 261120, 261121, 261122, 261123, 261124) c6(261125, 261126, 261127) c7(ID, bM, T, aC, aiH, fR, ea, if, e, al, eb, bB, fd, axk, ci, bh, mF, ip, wa, x, bv, aE, il, rJ, K, eL, hm, ly, yo, aW, hl, g, as, eF, ir, cm, bw, iP, ba, ou, eM, iA, AM, iT, cj, k, Hb, m, ym, pJ, Vj, dD, sw, zt, iB, aw, NT, adk, n, aj, oY, fr, qN, qW, cd, ai, ke, JX, ft, jz, zw, ql, h, lG, eV, hn, dL, vx, A, apo, sp, fe, aq, Og, mo, zZ, cM, lx, aCz, dB, hL, bm, hR, pg, fl, aDE, aZD, cB, B, AN, aEC, it, bL, apE, aZA, dA, mr, fG, jx, eX, aA, tc, hz, j, C, uW, V, q, J, eA, mj, RU, RI, hb, afQ, ld, yg, aT, cy, bq, aFm, ae, af, eu, qw, Uf, s, nO, aZB, oV, Y, bg, r, en, cl, t, ik, pb, vk, ck, nM, ac, aZC, iQ, gX, cS, ij, zu, bt, Ze, aDj, VB, hS, kh, id, gN, jh, vr, dG, Gn, eq, tK, mC, ad, hX, dP, fU, av, F, py, yt, NS, io, HD, JK, yF, ud, d, lx, lR, hG, et, qt, ff, eH, Of, eZ, kD, aeV, aoF, aF, BG, iV, bz, ek, LB, es, D, Bv, arY, pc, Ew, bE, aED, ll, gZ, aS, ME, y, ra, X); #c1(CXCL13) c2(NP_006410) c3(C-X-C motif chemokine 13 precursor) c4(164788) c5(164789, 164790, 164791, 164792, 164793, 164794) c6( ) c7(bM, T, dD, fM, Dw, fR, ea, fN, cz, jh, v, eq, apt, aDJ, AD, V, pd, mC, Pm, aZE, aoH, x, auO, Mo, aT, h, Ds, aJb, eL, hM, lx, aW, ff, az, aFK, Pg, eZ, en, iV, pl, cm, aAr, LB, y, eU, bP, aHL, U, cj, at, eo, k, yH, fL, fD, ij, hv); #c1 (CXCL14) c2(NP_004878) c3(C-X-C motif chemokine 14 precursor) c4(150495, 150496) c5(150497, 150498, 150499, 150500, 150501, 150502, 150503, 150504, 150505, 150506, 150507, 150508) c6( ) c7(bM, T, Dw, aC, dA, jx, aA, n, ci, q, ad, dP, av, aE, aT, h, Ds, K, ae, JI, aW, Y, vj, ff, r, aq, aF, en, g, apx, t, iK, bz, nM, nB, vA, at, lB, iu, cn); #c1(CXCL16) c2(NP_001094282) c3(C-X-C motif chemokine 16 precursor) c4(40649) c5(40650, 40651, 40652, 40653, 40654, 40655, 40656, 40657, 40658, 40659) c6( ) c7(oO, bM, T, dD, Dw, aC, eR, ea, e, cl, j, ci, eq, jO, fr, q, cd, o, ad, eA, bv, dY, aE, db, cy, Ds, hP, K, ae, eN, aZF, up, Ls, bg, hl, r, fe, aF, ba, es, ac, iA, dh, cS, aS, ei, y, B); #c1(CXCL17) c2(NP_940879) c3(VEGF coregulated chemokine 1 precursor) c4(260023) c5(260024, 260025, 260026, 260027, 260028, 260029, 260030, 260031, 260032, 260033) c6( ) c7(dA, if, PX, q, qN, aZG, bz, B); #c1(CXCL2) c2(NP_002080) c3(C-X-C motif chemokine 2 precursor) c4(124315) c5(124316, 124317, 124318, 124319, 124320, 124321) c6( ) c7(bM, Dw, wi, id, fG, ht, hE, cz, aZH, v, j, BF, C, atK, cd, eA, Bc, aT, cu, lG, Ds, cy, h, K, lQ, ji, bA, aa, uA, zH, Y, EU, fO, Np, Bw, wc, fJ, aZl, lo, ajz, hJ, eE, D, ac, uR, mx, wq, wR, aUS, fD, Pk, cB, y); #c1(CXCL3) c2(NP_002081) c3(C-X-C motif chemokine 3 precursor) c4(124322) c5(124323, 124324, 124325, 124326, 124327, 124328) c6( ) c7(bM, T, Dw, aZH, vr, BH, il, atK, jw, eA, Bc, aT, h, lG, Ds, K, lQ, uA, aa, aW, aU, EU, sP, t, aDs, uz, aUS, mk, qF, fD, Pk); #c1(CXCL5) c2(NP_002985) c3(C-X-C motif chemokine 5 precursor) c4(134190) c5(134191, 134192, 134193, 134194, 134195, 134196) c6( ) c7(bL, gh, T, bM, Dw, Mb, FR, dM, df, al, aA, n, aj, v, j, ci, q, J, ad, aZJ, dP, av, jz, ady, aE, Os, aT, Ds, vB, yb, ae, aa, ul, s, aW, Y, aZK, mQ, wc, ff, r, fe, lo, aF, en, g, iV, ik, bz, zV, LB, gk, eM, aDs, ki, dW, JH, fD, y, bt, qR, B); #c1 (CXCL6) c2(NP_002984) c3(C-X-C motif chemokine 6 precursor) c4(134183) c5(134184, 134185, 134186, 134187, 134188, 134189) c6( ) c7(aZJ, bM, Dw, y, zH, eM, Bc, Y, aw, fO, wc, eH, ao, lG, BH, Ds, zV, eF, t, ae, ad); #c1(CXCL8) c2(NP_000575) c3(interleukin-8 precursor) c4(12837) c5(12838, 12839, 12840, 12841, 12842, 12843) c6( ) c7(uB, gh, T, ea, fd, ci, bv, aE, rw, dV, BZ, Ds, Bi, zy, hH, cp, VF, aa, li, yo, az, ih, nY, zC, axp, GR, zV, ba, fT, gt, Jr, nB, dc, cj, Zn, Dt, k, aNn, fy, ul, cE, LT, nD, aFG, ht, aj, zo, bao, lG, ey, pl, MD, sp, fe, CQ, pm, apX, adz, aBs, avN, dB, ajd, XN, DD, aA, AO, bap, aXr, q, bag, zn, aZJ, cy, jj, dl, eu, le, bf, nO, bg, lo, cl, pb, Cv, cT, iM, ac, ig, AU, bt, rA, ao, BD, iW, aZU, rl, jN, jh, dG, Gn, ad, hX, iC, yt, oW, aHh, bac, lQ, WZ, cH, ff, aPK, Ce, tv, Sm, iV, bz, DF, baa, oQ, lf, lC, ra, ne, iu, oO, bM, if, e, bB, cL, lN, Tz, aZO, x, bah, bc, Qe, eG, JI, fc, hl, kt, NR, aoD, nr, as, eE, eF, cm, bw, qJ, xD, ki, vA, mx, hh, pJ, aTW, aZY, dD, zt, n, uE, bak, b, bae, zw, ec, Xe, gd, eV, hn, xP, A, bad, hT, lb, po, yq, amo, aZR, hl, at, pg, ut, fl, Ss, arO, B, dA, bab, wL, eX, fF, GC, v, V, akG, eJ, aZZ, hW, rS, sl, ae, aAi, tn, zH, iZ, aOK, t, ck, kP, ll, gX, kV, dM, df, aEt, OR, wV, eq, hd, py, ds, aCJ, hD, JK, nJ, atX, CL, d, je, et, aZK, wc, rM, dN, KD, Z, bF, D, gk, Da, dO, wz, Hs, aCP, y, kS, pC, aC, UH, fR, vf, atj, al, qj, bal, wa, AW, gJ, M, K, aZS, ajK, eL, adT, mH, HI, axu, aoo, Bw, XV, LZ, FU, aB, g, pL, uC, iP, aDs, aer, Gs, uo, dW, m, aZN, FW, sQ, sw, zX, aw, NT, BF, aZP, ft, Uh, gq, kH, cU, aaA, aDp, Ha, aTO, baj, BE, cM, GG, W, aZL, mg, hs, it, fM, fG, jx, aSE, j, eA, J, o, hb, Y, RF, Pg, r, ls, tu, en, ik, Dy, hc, azK, wR, dy, fD, LS, amL, Gl, sY, kh, id, LX, bS, ah, dP, fU, aZW, ji, adx, NM, WW, lR, eH, Sn, cr, aZM, gE, VI, hp, Fq, iR, baf, Nq, TA, eB, cP, hE, pf, atv, eb, zh, aER, YX, hm, aW, hJ, arZ, eM, aFc, fr, ct, qN, ai, wG, h, jy, dl, bam, pj, Nr, oo, qY, aDe, aq, kp, gG, ban, op, NL, hk, bm, hR, uz, bai, aZV, aMk, bL, Dw, eR, rf, cR, aBo, C, aCY, aZX, mP, qp, afQ, aT, dz, yb, aSX, an, af, aZA, alk, s, iU, aNi, akY, f, ajl, sS, aZT, nM, Zg, alt, vZ, lV, AH, wq, aSu, bH, Kd, qX, Ke, ZI, qb, aD, Me, vr, Jz, jE, pz, mC, kL, qC, av, F, oz, sO, NS, Os, aNR, ul, eZ, aEO, afr, aF, uF, ek, LB, eU, pc, zr, bE, z, uR, aS, XO, GF, X, Ex); #c1(CXCL9) c2(NP_002407) c3(C-X-C motif chemokine 9 precursor) c4(128150) c5(128151, 128152, 128153, 128154, 128155, 128156) c6( ) c7(bM, T, dD, pF, fM, fR, jp, dA, Dw, fN, jh, wV, eq, jE, fr, KO, pz, eA, eW, wa, F, ds, dV, aT, hD, h, Ds, iY, lQ, amY, bc, A, lU, je, aW, Y, ff, md, hl, wc, ge, cr, aq, lo, fe, en, iV, aaO, t, xe, f, eE, sS, OW, iM, eM, ll, ig, dc, hc, iT, fD, y, bt, B); #c1(CXCR1) c2(NP_000625) c3(C-X-C chemokine receptor type 1) c4(14054, 14055) c5(14056, 14057, 14058, 14059, 14060, 14061, 14062, 14063, 14064, 14065, 14066, 14067) c6( ) c7(bM, T, aC, fR, atj, al, bB, ci, aE, aa, JI, aW, az, nY, eF, uC, eM, aDs, ki, vA, uo, sQ, NT, aj, qW, ai, ft, wG, Uh, h, dL, cY, bar, qY, fe, Og, CO, op, lf, ajd, at, pg, ut, it, B, ka, dK, aA, J, aT, yb, aSX, ae, baq, Y, afq, ls, cl, pb, ck, ajl, DW, wq, ao, sY, df, aEt, ad, hX, dP, py, lC, lQ, WW, et, Of, eH, cr, aEO, kD, aF, uF, iV, bz, LB, pc, bE, y); #c1(CXCR2) c2(NP_00116/770) c3(C-X-C chemokine receptor type 2) c4(65950, 65951, 65952, 65953, 65954, 65955) c5(65956, 65957, 65958, 65959, 65960, 65961, 65962, 65963, 65964, 65965, 65966, 65967, 65968, 65969, 65970, 65971, 65972, 65973, 65974, 65975, 65976, 65977, 65978, 65979, 65980, 65981, 65982, 65983, 65984, 65985, 65986, 65987, 65988, 65989, 65990, 65991, 65992, 65993) c6( ) c7(bM, T, aC, ea, atj, fd, bB, ci, mF, aDI, AW, aE, Ds, K, hu, hH, Le, aa, DA, JI, aW, az, nY, g, as, hJ, eE, uC, lt, eM, aDs, AM, ki, bas, m, aTW, dD, ht, NT, n, fr, uE, ai, ft, Uh, cU, h, aDp, Ha, cY, Yd, qY, fe, kp, CQ, apX, lf, ajd, at, uz, pg, it, B, Dw, ka, Lx, jx, aA, afK, v, C, V, q, aT, cy, yb, aSX, ae, af, le, bf, Y, bg, r, cl, t, pb, aZu, fD, ao, nZ, sY, qb, df, vr, Jz, jE, eq, ad, dP, aHF, av, oz, ji, WW, d, et, cr, eZ, dN, kD, aF, un, Fq, iV, bz, DF, LB, pc, bE, aFb, Hs, lC, iu, y); #c1 (CXCR3) c2(NP_001136269) c3(C-X-C chemokine receptor type 3 isoform 2) c4(54743) c5(54744, 54745, 54746, 54747, 54748, 54749) c6( ) c7(bM, T, FR, fd, bB, cL, IN, wa, Ds, hH, bat, bc, cp, aa, aW, az, mQ, g, eE, eF, eM, Jr, iA, ki, mx, cj, k, m, dD, fr, kO, uE, ke, h, lG, xP, A, fJ, ap, fe, cM, xe, hk, at, Pr, Dw, ka, fM, avm, jx, fZ, v, j, bX, V, q, o, eA, zn, jZ, aT, cy, ae, hM, Y, r, lo, en, aaO, t, ck, cT, OW, ac, iM, ll, hc, Da, dy, fD, hS, aZ, iW, jh, wV, eq, fu, EA, ad, aoH, dP, hX, F, hD, vB, lQ, Od, NM, vP, uQ, je, lx, ff, eH, eZ, dN, hp, iV, aEx, Hv, es, bE, y, hv); #c1(CXCR3) c2(NP_001495) c3(C-X-C chemokine receptor type 3 isoform 1) c4(118193) c5(118194, 118195, 118196, 118197, 118198, 118199) c6( ) c7(bM, T, FR, fd, bB, cL, lN, wa, Ds, hH, bat, bc, cp, aa, aW, az, mQ, g, eE, eF, eM, Jr, iA, ki, mx, cj, k, m, dD, fr, kO, uE, ke, h, lG, xP, A, fJ, ap, fe, cM, xe, hk, at, Pr, Dw, ka, fM, avm, jx, fZ, v, j, bX, V, q, o, eA, zn, jZ, aT, cy, ae, hM, Y, r, lo, en, aaO, t, ck, cT, OW, ac, iM, ll, hc, Da, dy, fD, hS, aZ, iW, jh, wV, eq, fu, EA, ad, aoH, dP, hX, F, hD, vB, lQ, Od, NM, vP, uQ, je, lx, ff, eH, eZ, dN, hp, iV, aEx, Hv, es, bE, y, hv); #c1(CXCR3) c2(XP_005262314) c3(C-X-C chemokine receptor type 3 isoform X2) c4(277827) c5(277828, 277829, 277830, 277831, 277832, 277833) c6( ) c7(bM, T, FR, fd, bB, cL, IN, wa, Ds, hH, bat, bc, cp, aa, aW, az, mQ, g, eE, eF, eM, Jr, iA, ki, mx, cj, k, m, dD, fr, kO, uE, ke, h, lG, xP, A, fJ, ap, fe, cM, xe, hk, at, Pr, Dw, ka, fM, avm, jx, fZ, v, j, bX, V, q, o, eA, zn, jZ, aT, cy, ae, hM, Y, r, lo, en, aaQ, t, ck, cT, OW, ac, iM, ll, hc, Da, dy, fD, hS, aZ, iW, jh, wV, eq, fu, EA, ad, aoH, dP, hX, F, hD, vB, 1Q, Od, NM, vP, ul, je, lx, ff, eH, eZ, dN, hp, iV, aEx, Hv, es, bE, y, hv); #c1(CXCR4) c2(NP_001008540) c3(C-X-C chemokine receptor type 4 isoform a) c4(23325) c5(23326, 23327, 23328, 23329, 23330, 23331) c6( ) c7(oO, bM, T, os, lD, aC, fR, ea, if, EQ, fd, bB, ci, bh, aR, ip, wa, x, bv, xy, aE, K, hu, eq, hm, aa, ly, aEV, yo, aW, az, hl, aoD, g, as, ZE, eF, ir, Qt, cm, iP, bw, yd, bax, ou, CC, ba, eM, nB, AM, dc, iA, go, iT, cj, cX, k, m, ym, fy, Vj, sc, cZ, dD, lT, sw, zX, iB, ht, NT, n, oY, fr, qN, qW, ai, eW, BI, JX, ft, jz, azD, zw, ql, kH, h, hn, dL, bav, dd, KV, dx, OV, MD, Cw, CM, lb, fe, aq, Og, mo, cM, lx, ok, dB, NL, PI, ajd, bm, hR, pg, agy, apE, it, B, AN, aEC, bL, fM, dA, DD, Dh, fG, aA, tc, afK, v, j, C, AO, V, q, eA, eJ, RU, RI, nv, hb, Qg, rS, qK, yg, aT, cy, bau, ae, af, jf, qw, s, Qr, nO, oV, Y, iU, bg, r, lo, gm, eS, en, cl, LV, t, iK, pb, ck, baw, sS, baz, kP, Dy, ac, ig, nM, iM, iQ, gX, adR, np, fl, bt, zA, Ze, VB, hS, gV, kh, Dd, iW, jh, oZ, eq, hF, aue, ad, dP, av, hd, py, io, hD, bay, nJ, ul, d, lx, je, hG, et, qt, ff, eH, Of, eZ, kD, aeV, aoF, aF, iV, bz, dr, DF, ek, LB, es, OG, pc, bE, dO, aS, y); #c1(CXCR4) c2(NP_003458) c3(C-X-C chemokine receptor type 4 isoform b) c4(138463, 138464) c5(138465, 138466, 138467, 138468, 138469, 138470, 138471, 138472, 138473, 138474, 138475, 138476) c6( ) c7(oO, bM, T, os, lD, aC, fR, ea, if, EQ, fd, bB, ci, bh, aR, ip, wa, x, bv, xy, aE, K, hu, eL, hm, aa, ly, aEV, yo, aW, az, hl, aoD, g, as, zE, eF, ir, Qt, cm, iP, bw, yd, bax, ou, CC, ba, eM, nB, AM, dc, iA, go, iT, cj, cX, k, m, ym, fy, Vj, sc, cZ, dD, lT, sw, zX, iB, ht, NT, n, oY, fr, qN, qW, ai, eW, BI, JX, ft, jz, azD, zw, ql, kH, h, hn, dL, bav, dd, KV, dx, OV, MD, Cw, CM, lb, fe, aq, Og, mo, cM, lx, ok, dB, NL, Pl, ajd, bm, hR, pg, agy, apE, it, B, AN, aEC, bL, fM, dA, DD, Dh, fG, aA, tc, afK, v, j, C, AO, V, q, eA, eJ, RU, RI, nv, hb, Qg, rS, qK, yg, aT, cy, bau, ae, af, jf, qw, s, Ar, nO, oV, Y, iU, bg, r, lo, gm, eS, en, cl, LV, t, iK, pb, ck, baw, sS, baz, kP, Dy, ac, ig, nM, iM, iQ, gX, adR, np, fl, bt, zA, Ze, VB, hS, gV, kh, Dd, iW, jh, oZ, eq, hF, aue, ad, dP, av, hd, py, io, hD, bay, nJ, ul, d, lx, je, hG, et, qt, ff, eH, Of, eZ, kD, aeV, aoF, aF, iV, bz, dr, DF, ek, LB, es, OG, pc, bE, dO, aS, y); #c1 (CXCR5) c2(NP_001707) c3(C-X-C chemokine receptor type 5 isoform 1) c4(120290) c5(120291, 120292, 120293, 120294, 120295, 120296) c6( ) c7(T, fM, dA, fR, aD, dk, aw, iW, jh, fd, jE, ci, AO, V, pd, q, ad, hX, qC, av, x, F, aE, pe, aT, h, ae, bc, hM, lx, aW, az, aFK, eZ, r, kD, cm, aAr, bE, AV, cj, eo, k, y, apE); #c1(CXCR6) c2(NP_006555) c3(C-X-C chemokine receptor type 6) c4(166201) c5(166202, 166203, 166204, 166205, 166206, 166207, 166208, 166209, 166210, 166211, 166212, 166213, 166214, 166215, 166216, 166217, 166218, 166219) c6( ) c7(bL, bM, T, dD, iH, ea, yk, hU, ha, cz, fd, n, cl, v, j, ci, eq, fr, yp, ye, xY, o, cd, ad, ai, eA, yn, av, aE, db, xW, aeO, aT, hD, xV, h, bK, K, yb, yh, hP, yj, ae, yi, xX, d, xU, aW, Y, Ls, hl, yc, aq, aF, yq, t, nC, bz, ik, iP, dR, jq, ok, yf, bP, yl, il, xZ, do, cj, tl, at, cS, m, cP, ei, y, B); #c1(CXorf36) c2(NP_078965) c3(deleted in autism-related protein 1 isoform 2 precursor) c4(216292) c5(216293, 216294, 216295, 216296, 216297, 216298, 216299, 216300) c6(216301, 216302) c7(nf, VX, nA); #c1(CXorf36) c2(NP_789789) c3(deleted in autism-related protein 1 isoform 1 precursor) c4(252301) c5(252302, 252303, 252304, 252305, 252306, 252307, 252308, 252309) c6(252310, 252311) c7(nf, VX, nA); #c1(CXorf66) c2(NP_001013421) c3(uncharacterized protein CXorf66 precursor) c4(25532) c5(25533, 25534, 25535, 25536, 25537, 25538) c6( ) c7(he, bK); #c1(CXorf67) c2(NP_981952) c3(uncharacterized protein CXorf67) c4(263191) c5(263192, 263193, 263194, 263195, 263196, 263197) c6( ) c7(dB, Th, ad); #c1(CXXC1) c2(NP_001095124) c3(CXXC-type zinc finger protein 1 isoform 1) c4(40801) c5(40802, 40803, 40804, 40805, 40806, 40807) c6( ) c7(T, if, aA, fo, ci, qN, Qp, V, q, aR, eA, baA, bv, iu, hb, Xe, Qk, h, fb, iL, bA, TC, A, nO, Y, ff, xs, eH, eZ, r, nf, uY, fe, aF, en, eF, bN, Qi, nM, eM, agl, cj, B, fl); #c1(CXXC1) c2(NP_055408) c3(CXXC-type zinc finger protein 1 isoform 2) c4(180901) c5(180902, 180903, 180904, 180905, 180906, 180907) c6( ) c7(T, if, aA, fo, ci, qN, Qp, V, q, aR, eA, baA, bv, iu, hb, Xe, Qk, h, fb, iL, bA, TC, A, nO, Y, ff, xs, eH, eZ, r, nf, uY, fe, aF, en, eF, bN, Qi, nM, eM, agl, cj, B, fl); #c1(CXXC4) c2(NP_079488) c3(CXXC-type zinc finger protein 4) c4(218324) c5(218325, 218326, 218327, 218328, 218329, 218330, 218331, 218332, 218333, 218334) c6( ) c7(ck, iO, aA, aF, j, fr, br, bw, B, ad); #c1(CYB561) c2(XP_005257148) c3(cytochrome b561 isoform X1) c4(274710, 274711, 274712) c5(274713, 274714, 274715, 274716, 274717, 274718, 274719, 274720, 274721, 274722, 274723, 274724, 274725, 274726, 274727, 274728, 274729, 274730, 274731, 274732) c6( ) c7(il, cz); #c1(CYB561) c2(XP_011522726) c3(cytochrome b561 isoform X2) c4( ) c5(317260, 317261, 317262, 317263, 317264, 317265, 317266) c6( ) c7(il, cz); #c1(CYB561D2) c2(NP_001278213) c3(cytochrome b561 domain-containing protein 2) c4(109498) c5(109499, 109500, 109501, 109502, 109503, 109504, 109505, 109506, 109507, 109508, 109509, 109510, 109511, 109512, 109513, 109514, 109515, 109516) c6( ) c7(T, bz, BM); #c1 (CYB5A) c2(NP_001177736) c3(cytochrome b5 isoform 3) c4(72947) c5(72948, 72949, 72950, 72951, 72952, 72953) c6( ) c7(dP, aE, s, iU, axv, baC, adW, ti, baD, v, kp, K, uF, baB); #c1(CYB5A) c2(NP_683725) c3(cytochrome b5 isoform 1) c4(242527, 242528) c5(242529, 242530, 242531, 242532, 242533, 242534, 242535) c6( ) c7(dP, aE, s, iU, axv, baC, adW, ti, baD, v, kp, K, uF, baB); #c1(CYB5A) c2(XP_011524137) c3(cytochrome b5 isoform X1) c4(318692) c5(318693, 318694, 318695, 318696, 318697, 318698, 318699, 318700, 318701, 318702, 318703) c6( ) c7(dP, aE, s, iU, axv, baC, adW, ti, baD, v, kp, K, uF, baB); #c1(CYB5B) c2(NP_085056) c3(cytochrome b5 type B) c4(219235, 219236) c5(219237, 219238, 219239, 219240, 219241, 219242, 219243, 219244, 219245, 219246, 219247, 219248) c6( ) c7(eV, ex); #c1(CYBER3) c2(NP_000389) c3(NADH-cytochrome b5 reductase 3 isoform 1) c4(8400) c5(8401, 8402, 8403, 8404, 8405, 8406, 8407, 8408) c6( ) c7(AX, ck, T, adc, XX, eR, baE, baF, Y, axv, baD, lo, rk, nA, Kp); #c1(CYB5R3) c2(NP_001123291) c3(NADH-cytochrome b5 reductase 3 isoform 2) c4( ) c5(46843, 46844, 46845, 46846, 46847, 46848, 46849, 46850, 46851, 46852, 46853, 46854, 46855, 46856, 46857, 46858, 46859, 46860, 46861) c6( ) c7(AX, ck, T, adc, XX, eR, baE, baF, Y, axv, baD, lo, rK, nA, Kp); #c1(CYBER3) c2(NP_001165131) c3(NADH-cytochrome b5 reductase 3 isoform 3) c4(67511, 67512, 67513) c5(67514, 67515, 67516, 67517, 67518, 67519, 67520, 67521, 67522) c6( ) c7(AX, ck, T, adc, XX, eR, baE, baF, Y, axv, baD, lo, rK, nA, Kp); #c1(CYBER4) c2(NP_057314) c3(cytochrome b5 reductase 4) c4(190053, 190054) c5(190055, 190056, 190057, 190058, 190059, 190060, 190061, 190062, 190063, 190064, 190065, 190066) c6( ) c7(bL, mg, aj, mf, ey, K, J); #c1(CYBA) c2(NP_000092) c3(cytochrome b-245 light chain) c4(1668) c5(1669, 1670, 1671, 1672, 1673, 1674, 1675) c6( ) c7(bL, bM, Dw, dD, eR, QO, ea, iW, e, aXb, aw, aA, cz, n, aj, mw, j, Sv, fu, v, eb, aY, eq, Tz, J, dZ, Mg, nH, dY, baG, M, aT, lG, Ds, dL, K, bq, asC, eN, vl, eu, tw, sj, A, cH, aW, eZ, vs, aB, aF, bw, cM, pb, qa, ek, ck, kT, fT, wg, ac, hA, pE, eB, vy, gZ, uz, at, CZ, cS, dy, ei, lC, bt, zA, us, sZ); #c1(CYBB) c2(NP_000388) c3(cytochrome b-245 heavy chain) c4(8385, 8386, 8387) c5(8388, 8389, 8390, 8391, 8392, 8393, 8394, 8395, 8396, 8397, 8398, 8399) c6( ) c7(T, dD, id, dA, Fj, eR, qb, ea, aw, al, ew, cz, sM, cR, aj, bak, j, v, oY, PJ, eg, eq, cd, J, ai, x, azY, aE, sN, baH, Bc, dV, aT, h, lG, cy, dL, ey, baJ, K, bq, wt, af, VF, ul, wC, A, cW, aW, Cu, zb, fO, Y, oV, kD, fe, ek, aFA, bal, wg, ac, c, hA, dB, iT, wE, eB, at, Zn, wR, bal, dy, k, Hb, fD, ZA, jr); #c1(CYBB) c2(XP_011542192) c3(cytochrome b-245 heavy chain isoform X1) c4( ) c5(340663, 340664, 340665, 340666, 340667, 340668) c6( ) c7(T, dD, id, dA, Fj, eR, qb, ea, aw, al, ew, cz, sM, cR, aj, bak, j, v, oY, PJ, eg, eq, cd, J, ai, x, azY, aE, sN, baH, Bc, dV, aT, h, lG, cy, dL, ey, baJ, K, bq, wt, af, VF, ul, wC, A, cW, aW, Cu, zb, fO, Y, oV, kD, fe, ek, aFA, bal, wg, ac, c, hA, dB, iT, wE, eB, at, Zn, wR, bal, dy, k, Hb, fD, zA, jr); #c1(CYBRD1) c2(NP_001120855) c3(cytochrome b reductase 1 isoform 2) c4(44851) c5(44852, 44853, 44854, 44855, 44856, 44857) c6( ) c7(ac, yw, e, aw, r, yy, yu, j, ci, q, eu); #c1(CYBRD1) c2(NP_001243838) c3(cytochrome b reductase 1 isoform 3) c4(91809) c5(91810, 91811, 91812, 91813, 91814, 91815) c6( ) c7(ac, yw, e, aw, r, yy, yu, j, ci, q, eu); #c1(CYBRD1) c2(NP_079119) c3(cytochrome b reductase 1 isoform 1) c4(217168) c5(217169, 217/70, 217171, 217172, 217173, 217174) c6( ) c7(ac, yw, e, aw, r, yy, yu, j, ci, q, eu); #c1(CYC1) c2(NP_001907) c3(cytochrome cl, heme protein, mitochondrial precursor) c4(122133) c5(122134, 122135, 122136, 122137, 122138, 122139) c6( ) c7(t, baM, kl, eH, eF); #c1(CYCS) c2(NP_061820) c3(cytochrome c) c4(200497, 200498) c5(200499, 200500, 200501, 200502, 200503, 200504, 200505, 200506, 200507, 200508, 200509, 200510, 200511, 200512, 200513, 200514, 200515, 200516, 200517, 200518) c6( ) c7(baN, kh, BU, PK, xu, aW, fc, oW, aT, NI, zN, cy, v, DU, vs, Lz, y, nJ, eu); #c1(CYFIP1) c2(NP_001274739) c3(cytoplasmic FMRI-interacting protein 1 isoform a) c4(106720, 106721, 106722) c5(106723, 106724, 106725, 106726, 106727, 106728, 106729, 106730, 106731, 106732, 106733, 106734, 106735, 106736, 106737, 106738) c6( ) c7(ck, pr, hb, aNd, axd, fq, jj, aNe, rz, acD, kS, rc, aR, bN); #c1(CYFIP1) c2(XP_011542177) c3(cytoplasmic FMRI-interacting protein 1 isoform X1) c4(340607) c5(340608, 340609, 340610, 340611, 340612, 340613) c6( ) c7(ck, pr, hb, aNd, axd, fq, jj, aNe, rz, acD, KS, rc, aR, bN); #c1(CYFIP1) c2(XP_011542178) c3(cytoplasmic FMRI-interacting protein 1 isoform X2) c4(340614) c5(340615, 340616, 340617, 340618, 340619, 340620, 340621, 340622, 340623, 340624, 340625, 340626) c6( ) c7(ck, pr, hb, aNd, axd, fq, jj, aNe, rz, acD, KS, rc, aR, bN); #c1(CYFIP2) c2(NP_001278650) c3(cytoplasmic FMRI-interacting protein 2 isoform b) c4(109910, 109911) c5(109912, 109913, 109914, 109915, 109916, 109917, 109918) c6( ) c7(bM, T, bE, aW, sp, cj, aT, bB, m, Ku, t); #c1(CYFIP2) c2(NP_001278651) c3(cytoplasmic FMRI-interacting protein 2 isoform c) c4(109919, 109920) c5(109921, 109922, 109923, 109924, 109925, 109926, 109927, 109928, 109929, 109930, 109931, 109932) c6( ) c7(bM, T, bE, aW, sp, cj, aT, bB, m, Ku, t); #c1(CYFIP2) c2(XP_011532818) c3(cytoplasmic FMRI-interacting protein 2 isoform X1) c4(328943) c5(328944, 328945, 328946, 328947, 328948, 328949, 328950, 328951, 328952, 328953) c6( ) c7(bM, T, bE, aW, sp, cj, aT, bB, m, Ku, t); #c1(CYFIP2) c2(XP_011532819) c3(cytoplasmic FMRI-interacting protein 2 isoform X2) c4( ) c5(328954, 328955, 328956, 328957, 328958) c6( ) c7(bM, T, bE, aW, sp, cj, aT, bB, m, Ku, t); #c1(CYGB) c2(NP_599030) c3(cytoglobin) c4(235615) c5(235616, 235617, 235618, 235619, 235620, 235621) c6( ) c7(T, al, aj, Fb, ci, J, av, yw, aT, K, qD, d, aW, fQ, baP, g, t, ik, bz, aeb, bF, eM, dB, bal, dy, m, Hb); #c1(CYLC1) c2(NP_066941) c3(cylicin-1 isoform 1) c4(207623, 207624) c5(207625, 207626, 207627, 207628, 207629, 207630, 207631, 207632, 207633, 207634, 207635, 207636) c6( ) c7(h, T, iQ, jh); #c1(CYLC1) c2(XP_005262143) c3(cylicin-1 isoform X1) c4( ) c5(277753) c6( ) c7(h, T, iQ, jh); #c1(CYLC2) c2(NP_001331) c3(cylicin-2) c4(116990) c5(116991, 116992, 116993, 116994, 116995, 116996, 116997, 116998, 116999, 117000) c6( ) c7(aur, cF, aw, j, af); #c1(CYLD) c2(NP_001035877) c3(ubiquitin carboxyl-terminal hydrolase CYLD isoform 2) c4(33321) c5(33322, 33323, 33324, 33325, 33326, 33327, 33328, 33329, 33330, 33331) c6( ) c7(oO, T, dD, fM, fR, pf, ea, fG, yk, ht, atj, ex, aA, fd, j, ci, eq, Do, pd, ai, ad, eA, AW, Tf, qK, KH, aT, h, eV, hu, baU, azu, LC, Y, aw, bat, ajl, cr, ff, fe, af, en, g, bar, dr, bal, bf, ss, YE, Em, ajm, alt, hk, bas, fd, jq, y, aff, ce, B); #c1(CYLD) c2(NP_056062) c3(ubiquitin carboxyl-terminal hydrolase CYLD isoform 1) c4(185474, 185475) c5(185476, 185477, 185478, 185479, 185480, 185481, 185482, 185483, 185484, 185485, 185486, 185487) c6( ) c7(oO, T, dD, fM, fR, pf, ea, fG, yk, ht, atj, ex, aA, fd, j, ci, eq, Do, pd, ai, ad, eA, AW, Tf, qK, KH, aT, h, eV, hu, baU, azU, LC, Y, aW, baT, aJI, cr, ff, fe, aF, en, g, baR, dr, bad, bF, sS, YE, eM, aJm, alt, hk, baS, fD, jQ, y, aFf, cE, B); #c1(CYP11A1) c2(NP_000772) c3(cholesterol side-chain cleavage enzyme, mitochondrial isoform a precursor) c4(15555, 15556, 15557) c5(15558, 15559, 15560, 15561, 15562, 15563, 15564, 15565, 15566, 15567, 15568, 15569, 15570, 15571, 15572, 15573, 15574, 15575, 15576, 15577) c6( ) c7(bL, gh, aZ, My, baV, jN, cz, adW, it, dG, Ul, ml, eA, ad, UE, qC, dP, acu, aX, aT, iv, baX, uV, bq, ae, yi, avH, kl, s, aW, Pp, Hu, cl, zE, bN, baW, PI, FV, hl, Uv, va, cB); #c1(CYP11A1) c2(NP_001093243) c3(cholesterol side-chain cleavage enzyme, mitochondrial isoform b) c4(40147, 40148) c5(40149, 40150, 40151, 40152, 40153, 40154, 40155, 40156, 40157, 40158, 40159, 40160, 40161, 40162) c6( ) c7(bL, gh, aZ, My, baV, jN, cz, adW, it, dG, Ul, ml, eA, ad, UE, qC, dP, acu, aX, aT, iv, baX, uV, bq, ae, yi, avH, kl, s, aW, Pp, Hu, cl, zE, bN, baW, PI, FV, hL, Uv, va, cB); #c1(CYP11B1) c2(NP_000488) c3(cytochrome P450 11B1, mitochondrial isoform 1 precursor) c4(9836) c5(9837, 9838, 9839, 9840, 9841, 9842) c6( ) c7(Fj, bbf, iW, e, cz, j, MA, cd, bbc, qC, XD, aX, adX, aT, bbd, bbb, Fg, avH, s, baY, iw, bbe, baZ, Hu, aPz, dr, qa, bN, es, bba, iA, adZ, FV, jr); #c1 (CYP11B1) c2(NP_001021384) c3(cytochrome P450 11B1, mitochondrial isoform 2 precursor) c4(29150) c5(29151, 29152, 29153, 29154, 29155, 29156) c6( ) c7(Fj, bbf, iW, e, cz, j, MA, cd, bbc, qC, XD, aX, adX, aT, bbd, bbb, Fg, avH, s, baY, iw, bbe, baZ, Hu, aPz, dr, qa, bN, es, bba, iA, adZ, FV, jr); #c1(CYP11B2) c2(NP_000489) c3(cytochrome P450 (182, mitochondrial precursor) c4(9843) c5(9844, 9845, 9846, 9847, 9848, 9849) c6( ) c7(bL, dD, tf, bbi, Fj, mr, eR, wH, iW, oe, e, uv, aw, cz, eb, tc, bD, wV, j, uj, ah, ml, MA, aY, eq, JW, cd, bbc, bbg, cc, ec, MF, sN, bbh, M, aT, vg, iv, vc, ey, bbd, bbb, bq, sR, Fg, af, sj, kl, avH, Mr, lR, baY, s, ajT, iw, ul, bbe, Hu, mo, dw, uC, qa, es, KT, bba, ac, iA, bbj, dh, uR, mx, fP, uo, gZ, uz, uS, dy, wL, ei, ud, lC, sZ, y, vQ); #c1(CYP17A1) c2(NP_000093) c3(steroid 17-alpha-hydroxylase/17,20 lyase precursor) c4(1676) c5(1677, 1678, 1679, 1680, 1681, 1682) c6( ) c7(G, atT, T, Fj, aC, acz, e, ci, Ef, aE, u, il, aX, bbm, aW, hl, baC, nY, PQ, g, hJ, zE, uC, bbk, eM, iA, vA, Gs, cX, m, gn, aTW, FW, gU, uH, My, aw, NT, Wb, ml, hw, adX, jy, dL, aUm, uV, hB, Dz, RA, RT, Ln, fQ, aq, Pp, Hu, op, adZ, afc, anZ, hl, at, gv, B, bbl, bL, fM, eR, oe, jx, aee, aA, cz, j, q, UE, cF, ld, aT, bbd, an, Fg, ae, af, eu, bA, kl, s, Y, aeS, r, HL, eS, en, bu, cl, t, f, bN, sn, ac, aZ, bbn, iW, jh, ad, qC, dP, HF, l, F, vJ, acu, io, na, WZ, BO, Ub, PE, ff, eZ, aF, iR, bz, dr, es, gk, pu, yE, UB, aeg, Lb, be, y, aed, X, BA); #c1(CYP19A1) c2(NP_112503) c3(aromatase) c4(221187, 221188, 221189, 221190, 221191, 221192, 221193, 221194, 221195, 221196, 221197) c5(221198, 221199, 221200, 221201, 221202, 221203, 221204, 221205, 221206, 221207, 221208, 221209, 221210, 221211, 221212, 221213, 221214, 221215, 221216, 221217, 221218, 221219, 221220, 221221, 221222, 221223, 221224, 221225, 221226, 221227, 221228, 221229, 221230, 221231, 221232, 221233, 221234, 221235, 221236, 221237, 221238, 221239, 221240, 221241, 221242, 221243, 221244, 221245, 221246, 221247, 221248, 221249, 221250, 221251, 221252, 221253, 221254, 221255, 221256, 221257, 221258, 221259, 221260, 221261, 221262, 221263, 221264, 221265, 221266, 221267, 221268, 221269, 221270, 221271, 221272, 221273, 221274, 221275, 221276, 221277, 221278, 221279, 221280, 221281, 221282, 221283) c6( ) c7(G, T, UH, fR, pf, acz, if, e, al, cL, gf, ci, bbo, il, ari, iv, bbu, HR, qM, ake, iE, aW, fc, Np, g, eM, nB, vA, uo, cX, m, yA, aTW, Th, dD, gU, sQ, ht, NT, n, ml, qN, awq, bbt, hw, zf, lG, dL, aUm, uV, bbr, wC, yi, iw, aq, zZ, cM, Ld, op, afc, anZ, JA, Pl, mg, hL, at, ut, gv, B, qc, bL, aA, cz, v, j, bX, bbq, q, eA, UE, bbp, MP, aT, qv, Fx, bq, an, ae, af, bA, kl, bf, aeS, r, bbs, eS, cl, pt, t, f, bN, sn, aeM, gT, JO, hS, kh, aZ, iW, jN, uu, eq, ad, dP, av, hd, db, vJ, aet, hD, vc, lQ, nJ, aeI, d, PE, NH, eZ, aF, FX, bz, dr, yE, ag, auZ, sG, aeg, hE, aed, y); #c1(CYP1A1) c2(NP_000490) c3(cytochrome P450 (AI) c4(9850, 9851, 9852) c5(9853, 9854, 9855, 9856, 9857, 9858, 9859, 9860, 9861, 9862, 9863, 9864, 9865, 9866, 9867, 9868, 9869, 9870, 9871, 9872, 9873, 9874) c6( ) c7(bM, atT, T, aC, fR, ea, if, e, al, fd, bB, gf, ci, fw, sD, x, aEQ, Na, aE, u, lv, azm, K, aNZ, hu, hH, el, xQ, zQ, aW, mQ, hl, LZ, aB, g, eE, bw, uC, eM, nB, iA, nF, iT, vA, cj, NI, uo, cX, uJ, dW, k, m, Vj, iu, bbv, FW, gU, dD, sQ, aw, ht, NT, n, BH, aj, fr, qN, jw, ai, br, FY, co, ft, hw, Bj, Lv, lG, h, dL, eV, Gw, Dz, hB, ny, FZ, mR, fJ, AQ, aq, Pp, ajo, pm, cM, Ld, Zj, op, ar, bbz, W, dB, hk, bbx, PI, Qu, hL, at, uz, ut, gv, YH, zl, it, B, bL, jv, eR, jp, de, Lx, jx, eX, aA, cz, hy, v, j, acn, C, bX, Qo, lu, q, J, eA, eJ, XX, mP, afQ, aT, Nc, bq, an, ae, af, bA, kl, s, NE, nO, iU, Y, Lf, afS, r, Ox, lo, eS, en, bu, cl, t, Px, bbw, ik, bN, ck, sS, sn, c, ac, ig, bbA, fD, im, bt, rA, hS, GI, nZ, aZ, iW, ha, uO, jh, Gn, jE, eq, iG, hF, ad, kL, hX, dP, av, wD, l, hd, ib, db, yt, io, HD, ji, lQ, bby, aOh, WZ, IU, BO, Ub, cH, PE, cW, hG, ff, cr, eZ, Ej, aF, EZ, iV, bz, ek, TA, es, yE, bE, pP, gZ, dX, aS, Lb, Gu, oQ, X, rk); #c1(CYP1A2) c2(NP_000752) c3(cytochrome P450 1A2) c4(15321) c5(15322, 15323, 15324, 15325, 15326, 15327) c6( ) c7(bM, T, aC, fV, ea, e, al, bB, ci, bh, HH, gF, Na, aE, K, eL, bbC, aW, hl, g, bbD, cj, k, m, dD, NA, aw, ht, NT, n, ct, bbB, jz, Gw, dL, OM, Gc, ny, Nu, alt, fQ, aq, Ld, cv, W, Qu, hL, at, lB, B, bL, jp, aA, cz, hy, j, C, bX, q, eJ, XX, nv, aT, gz, an, ae, s, r, HL, en, bu, cl, t, ik, ac, c, bt, xb, GI, iW, ha, aHv, eq, uu, qi, ad, dP, av, HF, ib, PE, ff, aF, Fq, Z, bz, gk, adc, bE, Gu, QV, iu, y, BA); #c1(CYP1B1) c2(NP_000095) c3(cytochrome P450 (81) c4(1683) c5(1684, 1685, 1686, 1687, 1688, 1689, 1690, 1691, 1692, 1693) c6( ) c7(ID, bM, T, aC, if, e, bbH, fd, gf, ci, ip, x, aE, K, hu, eL, aa, ly, zQ, dH, aW, wF, g, bw, bbE, iA, vA, Gs, bbD, cj, cX, k, bbF, m, aTW, iu, asW, ht, ko, fr, ml, qN, jw, br, hw, dl, aUm, hB, bbG, ny, kj, aq, kp, dw, Ld, op, lx, kn, dB, anZ, Pl, hl, at, gv, JJ, sa, it, B, jv, fM, DD, aA, cz, hy, v, aSL, q, eA, aT, an, rz, ae, bA, avV, s, Y, r, eS, en, bu, cl, t, ik, bN, pZ, lJ, HQ, GI, iW, jh, Gn, ad, dP, av, ib, io, fb, nJ, lR, Ub, qt, PE, ff, eZ, aF, bz, es, yE, pP, aS, X); #c1(CYP21A2) c2(NP_000491) c3(steroid 21-hydroxylase isoform a) c4(9875, 9876, 9877, 9878) c5(9879, 9880, 9881, 9882, 9883, 9884, 9885, 9886, 9887, 9888, 9889, 9890, 9891, 9892, 9893, 9894, 9895, 9896, 9897, 9898, 9899, 9900, 9901, 9902, 9903, 9904, 9905, 9906, 9907, 9908, 9909, 9910, 9911, 9912, 9913, 9914) c6( ) c7(bbL, aZ, iW, aee, cz, v, ml, afD, Eh, bbc, KL, qC, b, aX, adX, bbl, uV, hK, Fg, dF, kl, avH, aiV, s, lC, iw, eZ, dq, aq, axx, Hu, mv, iR, cM, OU, dr, Wt, bbK, bN, amK, mO, y, lY, adZ, JQ, wz, cj, at, i, bbJ, B, ao); #c1(CYPZ1A2) c2(NP_001122062) c3(steroid 21-hydroxylase isoform b) c4(46350, 46351, 46352) c5(46353, 46354, 46355, 46356, 46357, 46358, 46359, 46360, 46361, 46362, 46363, 46364, 46365, 46366, 46367, 46368, 46369, 46370, 46371, 46372, 46373, 46374, 46375, 46376, 46377, 46378, 46379, 46380, 46381, 46382, 46383, 46384) c6( ) c7(bbL, aZ, iW, aee, cz, v, ml, afD, Eh, bbc, KL, qC, b, aX, adX, bbl, uV, hK, Fg, dF, kl, avH, aiV, s, lC, iw, eZ, dq, aq, axx, Hu, mv, iR, cM, OU, dr, Wt, bbK, bN, amK, mO, y, lY, adZ, JQ, wz, cj, at, i, bbJ, B, ao); #c1(CYP24A1) c2(NP_000773) c3(1,25-dihydroxyvitamin D(3) 24-hydroxylase, mitochondrial isoform 1 precursor) c4(15578) c5(15579, 15580, 15581, 15582, 15583, 15584) c6( ) c7(bM, T, iW, ht, Ay, bbO, n, oR, cz, j, ci, bX, aj, v, bB, V, qW, q, J, cd, ad, fU, amB, hd, av, TI, aE, iu, jz, zw, aT, dL, bbd, hu, hH, ae, azh, bbN, A, mR, qt, bbM, Y, aW, arp, r, aq, bbP, g, t, fr, bz, dr, qJ, arr, nM, eM, nB, bE, X, fP, at, dy, lS, B); #c1(CYP24A1) c2(NP_001122387) c3(1,25-dihydroxyvitamin D(3) 24-hydroxylase, mitochondrial isoform 2 precursor) c4(46586) c5(46587, 46588, 46589, 46590, 46591, 46592) c6( ) c7(bM, T, iW, ht, Ay, bbO, n, oR, cz, j, ci, bX, aj, v, bB, V, qW, q, J, cd, ad, fU, amB, hd, av, TI, aE, iu, jz, zw, aT, dL, bbd, hu, hH, ae, azh, bbN, A, mR, qt, bbM, Y, aW, arp, r, aq, bbP, g, t, fr, bz, dr, qJ, arr, nM, eM, nB, bE, X, fP, at, dy, lS, B); #c1(CYP26A1) c2(NP_000774) c3(cytochrome P450 26Al isoform 1) c4(15585) c5(15586, 15587, 15588, 15589, 15590, 15591, 15592, 15593, 15594, 15595, 15596, 15597, 15598, 15599, 15600) c6( ) c7(T, dD, ea, ht, eq, ci, dP, aT, j, af, mR, s, aW, Pg, LZ, g, cm, ik, bz, zV, ck, bF, pZ, lp, avF, dy, KS, X); #c1(CYP26A1) c2(NP_476498) c3(cytochrome P450 26A1 isoform 2) c4(230622) c5( ) c6( ) c7(T, dD, ea, ht, eq, ci, dP, aT, j, af, mR, s, aW, Pg, LZ, g, cm, ik, bz, zV, ck, bF, pZ, lp, avF, dy, kS, X); #c1(CYP26B1) c2(NP_001264671) c3(cytochrome P450 26B1 isoform 2) c4(97654) c5(97655, 97656, 97657, 97658, 97659, 97660) c6( ) c7(dD, ea, ff, Az, v, dy, eq, en, iV, cm, avn, zV, bbQ); #c1(CYP26B1) c2(NP_063938) c3(cytochrome P450 2681 isoform 1) c4(202244) c5(202245, 202246, 202247, 202248, 202249, 202250, 202251, 202252) c6(202253, 202254) c7(dD, ea, ff, Az, v, dy, eq, en, iV, cm, avn, zV, bbQ); #c1(CYP26C1) c2(NP_899230) c3(cytochrome P450 26C1) c4(258033) c5(258034, 258035, 258036, 258037, 258038, 258039) c6( ) c7(bbR, mR); #c1(CYP27Al) c2(NP_000775) c3(sterol 26-hydroxylase, mitochondrial precursor) c4(15601) c5(15602, 15603, 15604, 15605, 15606, 15607) c6( ) c7(bM, dD, ea, al, Vd, j, eq, bbS, ad, aR, dY, hB, dQ, H, an, ae, dH, Y, qS, aq, dg, hJ, eE, aFj, bbT, W, c, U, Lp, du, hE, B, L); #c1 (CYP27B1) c2(NP_000776) c3(25-hydroxyvitamin D-1 alpha hydroxylase, mitochondrial) c4(15608) c5(15609, 15610, 15611, 15612, 15613, 15614) c6( ) c7(bM, T, if, bB, cL, gf, qj, ci, bbZ, aY, Tz, xy, dV, K, bbV, hH, bc, Zm, azh, aa, aW, hl, LZ, g, eE, qJ, ba, BY, xK, rq, Jr, nB, eM, iA, vA, U, vy, PV, iS, azg, m, lS, Le, iu, bca, bbW, aYZ, aAP, lo, NT, xJ, aj, fr, qN, qW, cd, amB, jz, zw, aow, cU, h, dL, Nr, Dg, mR, A, OX, arp, ks, fe, bbX, aq, po, CO, KT, YE, mg, at, E, uz, va, it, B, bL, bbU, rG, bcb, eR, yk, cz, wo, v, j, C, V, q, J, o, eA, aT, aAR, jj, bq, hk, ae, bA, qw, s, Y, aCH, bg, r, aDm, rs, Lc, en, t, ck, ajl, arr, sS, BU, Dy, nM, ig, iQ, hr, wR, ars, gx, aZ, iW, Ay, ad, hX, av, abk, hd, F, v,J, hD, Dj, ji, nJ, yF, ul, d, Ub, PE, vj, ff, cr, eZ, gl, hp, iV, HI, bz, dr, Nq, es, yE, bE, bbY, Lj, Km, ei, y, X, pC); #c1(CYP27CI) c2(NP_001001665) c3(cytochrome P450 27C1) c4(18933) c5(18934, 18935, 18936, 18937, 18938, 18939, 18940, 18941, 18942) c6( ) c7(Hr); #c1(CYP27CI) c2(XP_006712546) c3(cytochrome P450 27C1 isoform X1) c4( ) c5(286720) c6( ) c7(Hr); #c1(CYP2A13) c2(NP_000757) c3(cytochrome P450 2A13) c4(15351) c5(15352, 15353, 15354, 15355, 15356, 15357, 15358, 15359, 15360, 15361, 15362, 15363, 15364, 15365, 15366, 15367, 15368, 15369) c6( ) c7(Gl, T, id, hy, bS, sD, aE, ja, Gw, dL, eL, s, Gv, ff, en, g, bz, dR, eM, vA, Gu, iu); #c1(CYP2A6) c2(NP_000753) c3(cytochrome P450 2A6 precursor) c4(15328, 15329, 15330) c5(15331, 15332, 15333, 15334, 15335, 15336) c6( ) c7(GI, T, jv, aqh, ha, bce, ht, aA, cz, bB, hy, bS, v, ct, ci, qN, it, bch, sD, q, ad, al, hX, av, ib, aE, hb, ja, aT, Vv, h, aNU, dL, gl, K, Gw, hu, eL, na, ny, s, JI, aW, Gv, A1, ff, hl, r, aq, en, g, Z, t, ik, bz, dR, gk, bcg, Dy, jx, eM, bE, bcf, X, bed, at, dy, bcc, Gu, xb, B); #c1(CYP2A7) c2(NP_000755) c3 (cytochrome P450 2A7 isoform 1 precursor) c4(15337) c5(15338, 15339, 15340, 15341, 15342, 15343) c6( ) c7(dB, T, aPm, s, jp); #c1(CYP2A7) c2(NP_085079) c3(cytochrome P450 2A7 isoform 2 precursor) c4(219263) c5(219264, 219265, 219266, 219267, 219268, 219269) c6( ) c7(dB, T, aPm, s, jp); #c1(CYP286) c2(NP_000758) c3(cytochrome P450 286 precursor) c4(15370) c5(15371, 15372, 15373, 15374, 15375, 15376, 15377) c6( ) c7(bM, T, os, aC, xS, ea, e, al, fd, gA, lR, ci, bh, UI, HH, bck, aY, aR, x, aED, aE, il, aX, bW, M, dV, iv, K, eL, bc, hm, eG, aiV, dH, aW, hC, az, hl, fc, bcj, hJ, eF, aaD, bw, zV, bbk, eM, nB, iT, vA, bbD, U, cj, dW, bcl, k, iu, dD, gU, oO, aw, aDI, aj, ct, ml, cd, eW, bbB, ke, qg, Bj, adX, h, bci, dL, uV, OM, he, ak, Nu, mR, auM, aq, kp, pm, dw, zZ, op, hq, iO, ok, adZ, dB, hk, FV, Uv, avF, YH, JJ, ben, B, bL, bbU, eR, QW, jx, aA, cz, ti, v, j, q, J, eA, cF, aT, cy, jj, bbd, Fg, ae, kl, s, iU, oV, Y, Lf, r, bbs, lo, HL, en, cl, t, f, pb, ck, sS, iQ, ac, wO, hc, bcm, gX, bt, lJ, ars, On, agh, aZ, ha, jN, jh, oZ, bS, eq, aXD, qi, hF, ad, wD, qC, dP, HF, av, F, oj, nJ, WZ, mu, d, hG, cH, Gv, ff, eH, eZ, gl, dq, aF, aDd, Fq, EZ, Z, bz, YR, dR, TA, gk, MJ, Lb, MC, hE, kS, CW, y); #c1(CYP286) c2(XP_011524849) c3(cytochrome P450 286 isoform X4) c4(319278) c5(319279, 319280, 319281, 319282, 319283) c6( ) c7(bM, T, os, aC, xS, ea, e, al, fd, gA, lR, ci, bh, UI, HH, bck, aY, aR, x, aEQ, aE, il, aX, bW, M, dV, iv, K, eL, bc, hm, eG, aiV, dH, aW, hC, az, hl, fc, bcj, hJ, eF, aaD, bw, zV, bbk, eM, nB, iT, vA, bbD, U, cj, dW, bcl, k, iu, dD, gU, oO, aw, aDl, aj, ct, ml, cd, eW, bbB, ke, qg, Bj, adX, h, bci, dL, uV, OM, he, ak, Nu, mR, auM, aq, kp, pm, dw, zZ, op, hq, iO, ok, adZ, dB, hk, FV, Uv, avF, YH, JJ, bcn, B, bL, bbU, eR, QW, jx, aA, cz, ti, v, j, q, J, eA, cF, aT, cy, jj, bbd, Fg, ae, kl, s, iU, oV, Y, Lf, r, bbs, lo, HL, en, cl, t, f, pb, ck, sS, iQ, ac, wl, hc, bcm, gX, bt, lJ, ars, On, agh, aZ, ha, jN, jh, oZ, bS, eq, aXD, qi, hF, ad, wD, qC, dP, HF, av, F, oj, nJ, WZ, mu, d, hG, cH, Gv, ff, eH, eZ, gl, dq, aF, aDd, Fq, EZ, Z, bz, YR, dR, TA, gk, MJ, Lb, MC, hE, kS, CW, y); #c1(CYP286) c2(XP_011524851) c3(cytochrome P450 286 isoform X6) c4(319284) c5(319285) c6( ) c7(bM, T, os, aC, xS, ea, e, al, fd, gA, lR, ci, bh, UI, HH, bck, aY, aR, x, aEQ, aE, il, aX, bW, M, dV, iv, K, eL, bc, hm, eG, aiV, dH, aW, hC, az, hl, fc, bcj, hJ, eF, aaD, bw, zV, bbk, eM, nB, iT, vA, bbD, U, cj, dW, bcl, k, iu, dD, gU, oO, aw, aDI, aj, ct, ml, cd, eW, bbB, ke, qg, Bj, adX, h, bci, dL, uV, OM, he, ak, Nu, mR, auM, aq, kp, pm, dw, zZ, op, hq, iO, ok, adZ, dB, hk, FV, Uv, avF, YH, JJ, ben, B, bL, bbU, eR, QW, jx, aA, cz, ti, v, j, q, J, eA, cF, aT, cy, jj, bbd, Fg, ae, kl, s, iU, oV, Y, Lf, r, bbs, lo, HL, en, cl, t, f, pb, ck, sS, iQ, ac, wl, hc, bcm, gX, bt, lJ, ars, On, agh, aZ, ha, jN, jh, oZ, bS, eq, aXD, qi, hF, ad, wD, qC, dP, HF, av, F, oj, nJ, WZ, mu, d, hG, cH, Gv, ff, eH, eZ, gl, dq, aF, aDd, Fq, EZ, Z, bz, YR, dR, TA, gk, MJ, Lb, MC, hE, KS, CW, y); #c1(CYP2B6) c2(XP_011524852) c3(cytochrome P450 286 isoform X7) c4(319286) c5(319287, 319288, 319289, 319290, 319291, 319292, 319293, 319294, 319295, 319296, 319297) c6( ) c7(bM, T, os, aC, xS, ea, e, al, fd, gA, lR, ci, bh, UI, HH, bck, aY, aR, x, aEQ, aE, il, aX, bW, M, dV, iv, K, eL, bc, hm, eG, aiV, dH, aW, hC, az, hl, fc, bcj, hJ, eF, aaD, bw, zV, bbk, eM, nB, iT, vA, bbD, U, cj, dW, bcl, k, iu, dD, gU, oO, aw, aDI, aj, ct, ml, cd, eW, bbB, ke, qg, Bj, adX, h, bci, dL, uV, OM, he, ak, Nu, mR, auM, aq, kp, pm, dw, zZ, op, hq, iO, ok, adZ, dB, hk, FV, Uv, avF, YH, JJ, bcn, B, bL, bbU, eR, QW, jx, aA, cz, ti, v, j, q, J, eA, cF, aT, cy, jj, bbd, Fg, ae, kl, s, iU, oV, Y, Lf, r, bbs, lo, HL, en, cl, t, f, pb, ck, sS, iQ, ac, wl, hc, bcm, gX, bt, lJ, ars, On, agh, aZ, ha, jN, jh, oZ, bS, eq, aXD, qi, hF, ad, wD, qC, dP, HF, av, F, oj, nJ, WZ, mu, d, hG, cH, Gv, ff, eH, eZ, gl, dq, aF, aDd, Fq, EZ, Z, bz, YR, dR, TA, gk, MJ, Lb, MC, HE, KS, CW, y); #c1(CYP2C18) c2(NP_000763) c3(cytochrome P450 2C18 isoform 1 precursor) c4(15420) c5(15421, 15422, 15423, 15424, 15425, 15426) c6( ) c7(T, eR, oe, ha, cz, j, ct, ad, Gt, ib, aT, af, ny, lR, aW, Au, aB, EZ, aaD, bz, ok, cj, B, On); #c1(CYP2C18) c2(NP_001122397) c3(cytochrome P450 2C18 isoform 2 precursor) c4(46664) c5(46665, 46666, 46667, 46668, 46669, 46670) c6( ) c7(T, eR, oe, ha, cz, j, ct, ad, Gt, ib, aT, af, ny, lR, aW, Au, aB, EZ, aaD, bz, ok, cj, B, On); #c1(CYP2C19) c2(NP_000760) c3(cytochrome P450 2019 precursor) c4(15378) c5(15379, 15380, 15381, 15382, 15383, 15384) c6( ) c7(bM, T, atj, e, al, lr, bO, fd, lj, gf, aY, Gt, il, fS, M, K, dQ, hH, eL, eN, hm, aW, az, hl, fc, nY, kt, Au, aB, bw, aaD, fT, eM, iT, qr, vy, cj, cE, dD, aw, ht, n, aj, oY, ct, ai, br, cc, adX, h, dL, DU, he, ny, ail, aq, zZ, cM, sE, hq, kT, ok, adZ, vb, at, E, uz, B, qc, bl, bcq, aCw, eR, wL, oe, aA, cz, aSE, v, j, q, qp, rS, aT, yb, rE, bau, dl, ne, r, iy, akY, en, t, iK, pb, ck, ac, ig, alt, gX, ao, gV, akd, qb, aD, ha, aET, bS, eq, ad, wD, hX, dP, av, ib, bco, uD, bcp, azS, lR, cH, ff, cr, eZ, kD, aF, aDd, EZ, Sm, Z, bz, iq, gk, aka, dX, Lb, hE, gq, y); #c1(CYP2C8) c2(NP_000761) c3(cytochrome P450 2C8 isoform a precursor) c4(15385, 15386, 15387) c5(15388, 15389, 15390, 15391, 15392, 15393, 15394, 15395, 15396, 15397, 15398, 15399, 15400, 15401, 15402, 15403, 15404, 15405) c6( ) c7(bL, gV, GI, dD, eR, jp, ea, e, ha, ht, aw, cz, fd, hy, j, ci, fu, bX, eq, aR, fU, Gt, pN, fm, fS, M, aT, oj, cy, oW, dV, Gw, de, dl, eu, hi, s, hG, aW, Au, aB, bct, aaD, bcr, t, ac, bo, bcs, cj, dX, dy, k, m, eg, Gu); #c1(CYP2C8) c2(NP_001185783) c3(cytochrome P450 2C8 isoform c) c4(76333) c5(76334, 76335, 76336, 76337, 76338, 76339, 76340, 76341, 76342, 76343, 76344, 76345, 76346, 76347, 76348, 76349, 76350, 76351) c6( ) c7(bL, gV, Gl, dD, eR, jp, ea, e, ha, ht, aw, cz, fd, hy, j, ci, fu, bX, eq, aR, FU, Gt, pN, fm, fS, M, aT, oj, cy, oW, dV, Gw, dQ, dl, eu, hi, s, hG, aW, Au, aB, bct, aaD, bcr, t, ac, bo, bes, cj, dX, dy, k, m, eg, Gu); #c1(CYP2C8) c2(NP_001185784) c3(cytochrome P450 208 isoform b) c4(76352, 76353) c5( ) c6( ) c7(bL, gV, Gl, dD, eR, jp, ea, e, ha, ht, aw, cz, fd, hy, j, ci, fu, bX, eq, aR, fU, Gt, pN, fm, fS, M, aT, oj, cy, oW, dV, Gw, dQ, dl, eu, hi, s, hG, aW, Au, aB, bct, aaD, bcr, t, ac, bo, bcs, cj, dX, dy, k, m, eg, Gu); #c1(CYP2C9) c2(NP_000762) c3(cytochrome P450 2C9 precursor) c4(15406, 15407) c5(15408, 15409, 15410, 15411, 15412, 15413, 15414, 15415, 15416, 15417, 15418, 15419) c6( ) c7(ID, bM, T, fV, ea, e, lr, bO, bB, lj, fd, bh, aR, Gt, vh, il, fS, M, hu, da, wt, aa, ly, aW, Az, hl, Au, aB, bct, aaD, fT, cj, wx, m, dD, aw, ht, ez, n, aj, oY, ct, cd, ai, cc, h, eV, Gw, dL, DU, ajV, FZ, aq, cM, lx, ok, dB, bo, E, uz, it, B, bL, aCw, eR, mr, wL, oe, uv, eX, aA, cz, hy, v, j, q, J, aT, cy, dl, kl, su, s, r, t, iK, pb, ac, ig, fP, dy, Tu, gV, GI, iW, ha, bS, eq, av, F, oj, uD, lR, cH, bcv, gC, EZ, Sm, Z, bz, qa, iq, bE, ag, gZ, bcu, dX, aS, bcc, Lb, Gu, y, X); #c1(CYP2D6) c2(NP_000097) c3(cytochrome P450 206 isoform 1) c4(1694, 1695, 1696, 1697, 1698, 1699) c5(1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735) c6( ) c7(bM, T, FR, wH, fV, if, e, al, bO, fd, bB, gf, ci, bh, HH, aY, gF, ip, Ef, x, fS, aU, aNZ, dQ, eL, xa, bc, hm, gr, ayT, yo, zQ, dH, aAu, aW, wf, hl, aaN, Qq, Au, g, Eg, JI, as, uC, yX, y, eM, nB, iT, U, cj, uo, dW, k, m, aCS, bcA, gL, FW, aEj, sQ, bcy, aw, n, aj, fr, ct, qN, cd, ai, FY, ke, b, ft, co, cc, h, dl, he, HO, A, mR, AQ, QP, aq, fe, dw, cM, cv, hq, ok, W, cA, lX, dB, hk, bo, FV, JM, VU, hR, E, ut, at, YH, lB, jd, ben, B, qc, bL, jv, aCw, bcB, iH, eR, wL, oe, hx, jx, aA, cz, tc, ti, hy, j, bcE, GC, V, J, aqs, aqN, ga, aVM, afQ, aql, aT, an, rE, lM, ae, af, dl, Su, ak, kl, avV, bcx, s, iU, Y, bcz, bcD, r, lo, aMA, HL, en, t, xw, ik, pb, bN, ck, sS, nM, iQ, pZ, hA, ig, bcw, fP, dy, bt, ao, vi, GI, VR, qb, ha, KO, bS, uu, jE, Gn, hF, ad, qC, av, HF, F, afm, bcC, oj, lQ, uD, na, hZ, BN, cH, PE, KS, ff, eZ, gl, aF, gu, Z, iR, bz, qa, gk, bE, pE, sG, gZ, dX, i, QV, ne, rk); #c1(CYP2D6) c2(NP_001020332) c3(cytochrome P450 206 isoform 2) c4(28624, 28625, 28626) c5(28627, 28628, 28629, 28630, 28631, 28632, 28633, 28634, 28635, 28636, 28637, 28638, 28639, 28640, 28641, 28642, 28643, 28644, 28645) c6( ) c7(bM, T, fR, wH, fV, if, e, al, bO, fd, bB, gf, ci, bh, HH, aY, gF, ip, Ef, x, fS, aU, aNZ, de, eL, xa, bc, hm, gr, ayT, yo, zQ, dH, aAu, aW, wf, hl, aaN, Qq, Au, g, Eg, JI, as, uC, yX, y, eM, nB, iT, U, cj, uo, dW, k, m, aCS, bcA, gL, FW, aEj, sQ, bcy, aw, n, aj, fr, ct, qN, cd, ai, FY, ke, b, ft, co, cc, h, dL, he, HO, A, mR, AQ, QP, aq, fe, dw, cM, cv, hq, ok, W, cA, lX, dB, hk, bo, FV, JM, VU, HR, E, ut, at, YH, lB, jd, ben, B, qc, bL, jv, aCw, bcB, iH, eR, wL, oe, hx, jx, aA, cz, tc, ti, hy, j, bcE, GC, V, J, aqs, aqN, ga, aVM, afQ, aql, aT, an, rE, lM, ae, af, dl, Su, ak, kl, avV, bcx, s, iU, Y, bcz, bcD, r, lo, aMA, HL, en, t, xw, iK, pb, bN, ck, sS, nM, iQ, pZ, hA, ig, bcw, fP, dy, bt, ao, vi, GI, VR, qb, ha, KO, bS, uu, jE, Gn, hF, ad, qC, av, HF, F, afm, bcC, oj, lQ, uD, na, hZ, BN, cH, PE, KS, ff, eZ, gl, aF, gu, Z, iR, bz, qa, gk, bE, pE, sG, gZ, dX, i, QV, ne, rk); #c1(CYP2D6) c2(XP_011528273) c3(cytochrome P450 206 isoform X8) c4(323348) c5( ) c6( ) c7(bM, T, FR, wHl, fV, if, e, al, bO, fd, bB, gf, ci, bh, HH, aY, gF, ip, Ef, x, fS, aU, aNZ, dQ, eL, xa, bc, hm, gr, ayT, yo, zQ, dH, aAu, aW, wf, hl, aaN, Qq, Au, g, Eg, JI, as, uC, yX, y, eM, nB, iT, U, cj, uo, dW, k, m, aCS, bcA, gL, FW, aEj, sQ, bcy, aw, n, aj, fr, ct, qN, cd, ai, FY, ke, b, ft, co, cc, h, dl, he, HO, A, mR, AQ, QP, aq, fe, dw, cM, cv, hq, ok, W, cA, lX, dB, hk, bo, FV, JM, VU, hR, E, ut, at, YH, lB, jd, ben, B, qc, bL, jv, aCw, bcB, iH, eR, wl, oe, hx, jx, aA, cz, tc, ti, hy, j, bcE, GC, V, J, aqs, aqN, ga, aVM, afQ, aql, aT, an, rE, lM, ae, af, dl, Su, ak, kl, avV, bcx, s, iU, Y, bcz, bcD, r, lo, aMA, HL, en, t, xw, iK, pb, bN, ck, sS, nM, iQ, pZ, hA, ig, bcw, fP, dy, bt, ao, vi, GI, VR, qb, ha, KO, bS, uu, jE, Gn, hF, ad, qC, av, HF, F, afm, bcC, oj, lQ, uD, na, hZ, BN, CH, PE, KS, ff, eZ, gl, aF, gu, Z, iR, bz, qa, gk, bE, pE, sG, gZ, dX, i, QV, ne, rk); #c1 (CYP2D6) c2(XP_011528274) c3(cytochrome P450 206 isoform X5) c4( ) c5(323349, 323350, 323351, 323352, 323353) c6( ) c7(bM, T, fR, wH, fV, if, e, al, bO, fd, bB, gf, ci, bh, HH, aY, gF, ip, Ef, x, fS, aU, aNZ, dQ, eL, xa, bc, hm, gr, ay T, yo, zQ, dH, aAu, aW, wf, hl, aaN, Qq, Au, g, Eg, JI, as, uC, yX, y, eM, nB, iT, U, cj, uo, dW, k, m, aCS, bcA, gL, FW, aEj, sQ, bcy, aw, n, aj, fr, ct, qN, cd, ai, FY, ke, b, ft, co, cc, h, dl, he, HO, A, mR, AQ, QP, aq, fe, dw, cM, cv, hq, ok, W, cA, lX, dB, hk, bo, FV, JM, VU, hR, E, ut, at, YH, lB, jd, ben, B, qc, bL, jv, aCw, bcB, iH, eR, wL, oe, hx, jx, aA, cz, tc, ti, hy, j, bcE, GC, V, J, aqs, aqN, ga, aVM, afd, aql, aT, an, rE, lM, ae, af, dl, Su, ak, kl, avV, bcx, s, iU, Y, bcz, bcD, r, lo, aMA, HL, en, t, xw, iK, pb, bN, ck, sS, nM, iQ, pZ, hA, ig, bcw, fP, dy, bt, ao, vi, GI, VR, qb, ha, KO, bS, uu, jE, Gn, hF, ad, qC, av, HF, F, afm, bcC, oj, lQ, uD, na, hZ, BN, cH, PE, KS, ff, eZ, gl, aF, gu, Z, iR, bz, qa, gk, bE, pE, sG, gZ, dX, i, QV, ne, rk); #c1(CYP2E1) c2(NP_000764) c3(cytochrome P450 2El precursor) c4(15427, 15428) c5(15429, 15430, 15431, 15432, 15433, 15434, 15435, 15436, 15437, 15438, 15439, 15440, 15441, 15442, 15443, 15444) c6( ) c7(bM, atT, T, uk, fV, ea, e, al, if, lj, ci, bh, fw, sD, x, Na, AW, Qk, K, aDZ, hu, LG, dF, eL, JI, aW, az, hl, fc, kt, aB, g, bw, uC, yd, fT, eM, nF, Gs, mx, cj, dW, bcl, k, gn, m, pw, abh, FW, dD, Ol, ht, NT, aj, ct, qN, cd, ai, FY, ft, cc, Bj, h, lG, aNU, dL, eV, ZN, ajV, hi, ny, A, AQ, aq, bcG, ar, W, dB, Qu, at, uz, NB, lB, it, B, bL, bcF, jv, jp, oe, jx, eX, aA, cz, hy, v, j, C, bX, q, J, eA, Yn, aT, cy, Nc, bq, an, ae, avV, s, Y, r, en, t, ik, pb, tR, ck, BU, ig, bcH, fD, bt, Gl, id, aD, iW, jh, Gd, bS, eq, iG, hF, ad, kL, dP, av, F, ib, NS, il, na, Gv, ff, eZ, aF, Sm, Z, iR, bz, Oh, TA, es, gk, Zi, gZ, cP, i, hE, iu, rk); #c1(CYP2F1) c2(NP_000765) c3(cytochrome P450 2F1 precursor) c4(15445) c5(15446, 15447, 15448, 15449, 15450, 15451) c6( ) c7(at, av, bz, jp); #c1(CYP2RI) c2(NP_078790) c3(vitamin D 25-hydroxylase) c4(215477) c5(215478, 215479, 215480, 215481, 215482, 215483) c6( ) c7(bM, aec, al, gN, bB, hz, fr, Ul, V, q, eA, ad, F, aE, bcl, hD, h, ae, A, ND, Y, r, dr, qJ, nM, bE, mg, y, B, ars); #c1(CYP2S1) c2(NP_085125) c3(cytochrome P450 2SI precursor) c4(219310, 219311, 219312) c5(219313, 219314, 219315, 219316, 219317, 219318, 219319, 219320, 219321, 219322, 219323, 219324, 219325, 219326, 219327, 219328, 219329, 219330) c6( ) c7(IQ, cr, cz); #c1(CYP2U1) c2(NP_898898) c3(cytochrome P450 2U1) c4(257844, 257845) c5(257846, 257847, 257848, 257849, 257850, 257851, 257852, 257853, 257854, 257855, 257856, 257857) c6( ) c7(bcJ); #c1(CYP2WI) c2(NP_060251) c3(cytochrome P450 2Wl precursor) c4(195034) c5(195035, 195036, 195037, 195038, 195039, 195040) c6( ) c7(T, B, bE, aW, r, aT, bB, Hu, q, X); #c1(CYP39A1) c2(NP_057677) c3(24-hydroxycholesterol 7-alpha-hydroxylase isoform 1) c4(192310) c5(192311, 192312, 192313, 192314, 192315, 192316) c6( ) c7(gV, bck, K, aFj, hG); #c1(CYP3A4) c2(NP_001189784) c3(cytochrome P450 3A4 isoform 2) c4( ) c5(80264, 80265, 80266, 80267, 80268) c6( ) c7(bM, G, T, Zz, ea, al, bB, gf, ci, bh, x, il, fS, dV, K, de, eL, bc, zQ, aW, fc, hl, g, lm, hJ, eF, bw, eM, nF, Gs, dW, k, m, pw, cE, dD, aw, ht, aj, fr, jG, qN, alc, ft, gd, adX, h, eV, Gw, hn, dL, HO, bbN, hi, aix, aq, cM, bbT, hq, iO, adZ, hk, aZL, FV, hL, gg, jd, B, bL, eR, jx, ex, cz, hy, v, j, bX, q, J, aT, vH, ae, bA, iU, ev, r, HL, en, bu, cl, t, ck, BU, iQ, ac, gX, fP, dy, bt, ao, On, gV, Gl, ha, jh, uu, ah, eq, gb, ad, hX, av, HF, hd, F, ib, oj, aak, fb, hG, cH, ff, gu, iV, bz, ek, es, gk, bE, Lb, cP, ei, i, Gu, X); #c1(CYP3A4) c2(NP_059488) c3(cytochrome P450 3A4 isoform 1) c4(193323, 193324) c5(193325, 193326, 193327, 193328, 193329, 193330, 193331, 193332, 193333, 193334, 193335, 193336, 193337) c6( ) c7(bM, G, T, Zz, ea, al, bB, gf, ci, bh, x, il, fS, dV, K, dQ, eL, bc, zQ, aW, fc, hl, g, lm, hJ, eF, bw, eM, nF, Gs, dW, k, m, pw, cE, dD, aw, ht, aj, fr, jG, qN, alc, ft, gd, adX, h, eV, Gw, hn, dL, HO, bbN, hi, aix, aq, cM, bbT, hq, iO, adZ, hk, aZL, FV, hL, gg, jd, B, bL, eR, jx, eX, cz, hy, v, j, bX, q, J, aT, vH, ae, bA, iU, ev, r, HL, en, bu, cl, t, ck, BU, iQ, ac, gX, fP, dy, bt, ao, On, gV, Gl, ha, jh, uu, ah, eq, gb, ad, hX, av, HF, hd, F, ib, oj, aak, fb, hG, cH, ff, gu, iV, bz, ek, es, gk, bE, Lb, cP, ei, i, Gu, X); #c1(CYP3A4) c2(XP_011514144) c3(cytochrome P450 3A4 isoform X2) c4(306737) c5( ) c6( ) c7(bM, G, T, Zz, ea, al, bB, gf, ci, bh, x, il, fS, dV, K, de, eL, bc, zQ, aW, fc, hl, g, lm, hJ, eF, bw, eM, nF, Gs, dW, k, m, pw, cE, dD, aw, ht, aj, fr, jG, qN, alc, ft, gd, adX, h, eV, Gw, hn, dL, HO, bbN, hi, aix, aq, cM, bbT, hq, iO, adZ, hk, aZL, FV, hL, gg, jd, B, bL, eR, jx, ex, cz, hy, v, j, bX, q, J, aT, vH, ae, bA, iU, ev, r, HL, en, bu, cl, t, ck, BU, iQ, ac, gX, fP, dy, bt, ao, On, gV, Gl, ha, jh, uu, ah, eq, gb, ad, hX, av, HF, hd, F, ib, oj, aak, fb, hG, cH, ff, gu, iV, bz, eK, es, gk, bE, Lb, cP, ei, i, Gu, X); #c1(CYP3A43) c2(NP_001265850) c3(cytochrome P450 3A43 isoform 4) c4(99484, 99485, 99486) c5(99487, 99488, 99489, 99490, 99491) c6( ) c7(aW, ae, ad, aT); #c1(CYP3A43) c2(NP_073731) c3(cytochrome P450 3A43 isoform 1) c4(213305) c5(213306, 213307, 213308, 213309, 213310, 213311) c6( ) c7(aW, ae, ad, aT); #c1(CYP3A43) c2(NP_476436) c3(cytochrome P450 3A43 isoform 2) c4(230594, 230595) c5(230596, 230597, 230598, 230599, 230600, 230601, 230602) c6( ) c7(aW, ae, ad, aT); #c1(CYP3A43) c2(NP_476437) c3(cytochrome P450 3A43 isoform 3) c4(230603, 230604) c5(230605, 230606, 230607, 230608, 230609, 230610, 230611, 230612, 230613, 230614, 230615, 230616, 230617, 230618, 230619, 230620, 230621) c6( ) c7(aW, ae, ad, aT); #c1(CYP3A43) c2(XP_011514795) c3(cytochrome P450 3A43 isoform X1) c4(307439) c5( ) c6( ) c7(aW, ae, ad, aT); #c1(CYP3A43) c2(XP_011514796) c3(cytochrome P450 3A43 isoform X2) c4(307440) c5(307441, 307442, 307443, 307444, 307445, 307446) c6( ) c7(aW, ae, ad, aT); #c1(CYP3A5) c2(NP_000768) c3(cytochrome P450 3A5 isoform 1) c4(15452) c5(15453, 15454, 15455, 15456, 15457, 15458) c6( ) c7(bM, G, T, e, lj, ci, gF, x, il, fS, dV, de, aW, hJ, ou, eM, iT, nF, k, m, fy, dD, aw, n, aj, fr, qN, ft, h, dL, RT, aq, cM, W, hk, at, va, gg, it, B, bL, aCw, eR, jp, oe, cz, fF, hy, j, bX, q, ga, aT, ae, iU, bcL, Lf, r, HL, en, bu, t, ck, ac, gX, fP, dy, bt, gV, ha, eq, jE, fu, ad, fU, av, hd, oj, fb, gj, lU, hG, cH, ff, gu, iV, gW, bz, qa, iq, es, Lb, i); #c1(CYP3A5) c2(NP_001177413) c3(cytochrome P450 3A5 isoform 2) c4(72782) c5(72783, 72784, 72785, 72786, 72787, 72788, 72789, 72790, 72791) c6( ) c7(bM, G, T, e, lj, ci, gF, x, il, fS, dV, dQ, aW, hJ, ou, eM, iT, nF, k, m, fy, dD, aw, n, aj, fr, qN, ft, h, dL, RT, aq, cM, W, hk, at, va, gg, it, B, bL, aCw, eR, jp, oe, cz, fF, hy, j, bX, q, ga, aT, ae, iU, bcl, Lf, r, HL, en, bu, t, ck, ac, gX, fP, dy, bt, gV, ha, eq, jE, fu, ad, fU, av, hd, oj, fb, gj, lU, hG, cH, ff, gu, iV, gW, bz, qa, iq, es, Lb, i); #c1(CYP3A5) c2(NP_001278759) c3(cytochrome P450 3A5 isoform 4 precursor) c4(110051) c5(110052, 110053, 110054, 110055, 110056, 110057) c6( ) c7(bM, G, T, e, lj, ci, gF, x, il, fS, dV, de, aW, hJ, ou, eM, iT, nF, k, m, fy, dD, aw, n, aj, fr, qN, ft, h, dL, RT, aq, cM, W, hk, at, va, gg, it, B, bL, aCw, eR, jp, oe, cz, fF, hy, j, bX, q, ga, aT, ae, iU, bcl, Lf, r, HL, en, bu, t, ck, ac, gX, fP, dy, bt, gV, ha, eq, jE, fu, ad, fU, av, hd, oj, fb, gj, lU, hG, cH, ff, gu, iV, gW, bz, qa, iq, es, Lb, i); #c1(CYP3A5) c2(XP_011514149) c3(cytochrome P450 3A5 isoform X3) c4( ) c5(306738) c6( ) c7(bM, G, T, e, lj, ci, gF, x, il, fS, dV, dQ, aW, hJ, ou, eM, iT, nF, k, m, fy, dD, aw, n, aj, fr, qN, ft, h, dL, RT, aq, cM, W, hk, at, va, gg, it, B, bL, aCw, eR, jp, oe, cz, fF, hy, j, bX, q, ga, aT, ae, iU, bcL, Lf, r, HL, en, bu, t, ck, ac, gX, fP, dy, bt, gV, ha, eq, jE, fu, ad, fU, av, hd, oj, fb, gj, lU, hG, cH, ff, gu, iV, gW, bz, qa, iq, es, Lb, i); #c1 (CYP3A7) c2(NP_000756) c3(cytochrome P450 3A7) c4(15344) c5(15345, 15346, 15347, 15348, 15349, 15350) c6( ) c7(bM, kl, aW, aZ, Lf, aT, fQ, aq, at, dL, m, bu, bz, B, ad, c1); #c1(CYP3A7-CYP3A5IP) c2(NP_001243426) c3(CYP3A7-CYP3ASIP protein) c4(90763) c5(90764, 90765, 90766, 90767, 90768, 90769) c6( ) c7(bM, gj, kl, aW, aZ, Lf, aT, fF, aq, bu, bz, B, c1); #c1(CYP46Al) c2(NP_006659) c3(cholesterol 24-hydroxylase precursor) c4(166972, 166973) c5(166974, 166975, 166976, 166977, 166978, 166979, 166980, 166981, 166982, 166983, 166984, 166985) c6( ) c7(cZ, W, dH, sO, dd, H, af, L); #c1(CYP4All) c2(XP_011539132) c3 (cytochrome P450 4All isoform X6) c4(337209) c5(337210, 337211, 337212, 337213, 337214) c6( ) c7(ac, eu, aw, M, cz, cR, aq, j, ek, bq, uW, ye, B, qa, um); #c1(CYP4BI) c2(XP_011539133) c3(cytochrome P450 4B1 isoform X1) c4(337215) c5(337216, 337217, 337218, 337219, 337220, 337221) c6( ) c7(av, T, eW, d, aW, cj, aT, cz, dL, eA, eL, B); #c1(CYP4F11) c2(NP_067010) c3(phylloquinone omega-hydroxylase CYP4F11 precursor) c4(208310, 208311) c5(208312, 208313, 208314, 208315, 208316, 208317, 208318, 208319, 208320, 208321, 208322, 208323, 208324, 208325, 208326, 208327) c6( ) c7(aW, aT); #c1(CYP4F1l) c2(XP_011526458) c3(phylloquinone omega-hydroxylase CYP4F11 isoform X1) c4( ) c5(320760) c6( ) c7(aW, aT); #c1(CYP4F12) c2(NP_076433) c3(cytochrome P450 4F12) c4( ) c5(214156, 214157, 214158, 214159, 214160, 214161, 214162, 214163) c6( ) c7(bX, cz); #c1(CYP4F12) c2(XP_011526501) c3(cytochrome P450 4F12 isoform X1) c4(320798) c5(320799, 320800) c6( ) c7(bX, cz); #c1(CYP4F12) c2(XP_011526503) c3(cytochrome P450 4F12 isoform X3) c4(320801) c5( ) c6( ) c7(bX, cz); #c1(CYP4F12) c2(XP_011526508) c3(cytochrome P450 4F12 isoform X6) c4( ) c5(320802, 320803, 320804, 320805, 320806, 320807) c6( ) c7(bX, cz); #c1(CYP4F2) c2(NP_001073) c3(phylloquinone omega-hydroxylase CYP4F2 precursor) c4(36899, 36900, 36901) c5( ) c6( ) c7(ac, mr, cH, wL, aw, M, EQ, cz, kp, aS, hp, ei, aaD, eu, B, qa, ek); #c1(CYP4F22) c2(NP_775754) c3(cytochrome P450 4F22) c4(250175) c5(250176, 250177, 250178, 250179, 250180, 250181) c6( ) c7(fn, y, bcM, bcj); #c1(CYP4F3) c2(NP_000887) c3(docosahexaenoic acid omega-hydroxylase CYP4F3 isoform a) c4(16844) c5(16845, 16846, 16847, 16848, 16849, 16850) c6( ) c7(bM, T, aw, aA, cz, v, ci, HH, V, q, ad, ip, HF, fS, aT, K, jj, Fg, ae, nJ, aW, oV, ff, fc, hl, r, aq, lo, kp, hp, aF, en, cl, as, EZ, HL, bcN, bz, qa, op, iO, bbz, bbx, vA, hL, cP, kS, B); #c1(CYP4F3) c2(NP_001186138) c3(docosahexaenoic acid omega-hydroxylase CYP4F3 isoform b) c4(77197, 77198) c5(77199, 77200, 77201, 77202, 77203, 77204, 77205, 77206, 77207, 77208, 77209, 77210) c6( ) c7(bM, T, aw, aA, cz, v, ci, HH, V, q, ad, ip, HF, fS, aT, K, jj, Fg, ae, nJ, aW, oV, ff, fc, hl, r, aq, lo, kp, hp, aF, en, cl, as, EZ, HL, bcN, bz, qa, op, iO, bbz, bbx, vA, hL, cP, kS, B): #c1 (CYP4F8) c2(NP_009184) c3(cytochrome P450 4F8 precursor) c4(171349) c5(171350, 171351, 171352, 171353, 171354, 171355, 171356, 171357, 171358, 171359, 171360, 171361, 171362, 171363, 171364, 171365) c6( ) c7(ae, ad); #c1(CYP4F8) c2(XP_011525956) c3(cytochrome P450 4F8 isoform X2) c4(320349, 320350) c5(320351, 320352) c6( ) c7(ae, ad); #c1(CYP4V2) c2(NP_997235) c3(cytochrome P450 4V2) c4(265247, 265248) c5(265249, 265250, 265251, 265252, 265253, 265254, 265255, 265256, 265257, 265258, 265259, 265260) c6( ) c7(P, cj, kf, nm, h, bci, bcO); #c1(CYP51A1) c2(NP_000777) c3(lanosterol 14-alpha demethylase isoform 1 precursor) c4(15615, 15616, 15617) c5(15618, 15619, 15620, 15621, 15622, 15623, 15624, 15625, 15626, 15627, 15628, 15629, 15630, 15631, 15632) c6( ) c7(avF, wR, T, aiV, qE); #c1(CYP51A1) c2(NP_001139624) c3(lanosterol 14-alpha demethylase isoform 2) c4( ) c5(59566, 59567, 59568, 59569, 59570) c6( ) c7(avF, wR, T, aiV, qE); #c1(CYP7Al) c2(NP_000771) c3(cholesterol 7-alpha-monooxygenase) c4(15459) c5(15460, 15461, 15462, 15463, 15464, 15465, 15466, 15467, 15468, 15469, 15470, 15471, 15472, 15473, 15474, 15475, 15476, 15477, 15478, 15479, 15480, 15481, 15482, 15483, 15484, 15485, 15486, 15487, 15488, 15489, 15490, 15491, 15492, 15493, 15494, 15495, 15496, 15497, 15498, 15499, 15500, 15501, 15502, 15503, 15504, 15505, 15506, 15507, 15508, 15509, 15510, 15511, 15512, 15513, 15514, 15515, 15516, 15517, 15518, 15519, 15520, 15521, 15522, 15523, 15524, 15525, 15526, 15527, 15528, 15529, 15530, 15531, 15532, 15533, 15534, 15535, 15536, 15537, 15538, 15539, 15540, 15541, 15542, 15543, 15544, 15545, 15546, 15547, 15548, 15549, 15550, 15551, 15552, 15553, 15554) c6( ) c7(dD, Zz, ea, oe, e, aw, cz, bB, aj, v, j, eq, q, J, jF, l, dY, u, ec, il, io, dL, K, hB, dQ, bc, cH, cW, hl, r, dq, aq, hJ, bcP, bN, bJ, cO, bE, hr, B); #c1(CYP7B1) c2(NP_004811) c3(25-hydroxycholesterol 7-alpha-hydroxylase) c4(149987) c5(149988, 149989, 149990, 149991, 149992, 149993) c6( ) c7(bcR, Ng, ad, aV, lu, bcS, nu, fc, hl, at, bck, bcd, dl, Fb, g, hJ, y, ae, af, bcT, afj); #c1(CYP8B1) c2(NP_004382) c3(7-alpha-hydroxycholest-4-en-3-one 12-alpha-hydroxylase) c4(145971) c5(145972, 145973, 145974, 145975, 145976, 145977) c6( ) c7(jF, aj, hJ, J, bX); #c1(CYS1) c2(NP_001032237) c3(cystin-1) c4(31111) c5(31112, 31113, 31114, 31115, 31116, 31117) c6( ) c7(vm); #c1(CYSLTR1) c2(NP_001269117) c3(cysteinyl leukotriene receptor 1) c4(100474) c5(100475, 100476, 100477, 100478, 100479, 100480) c6( ) c7(bM, T, dD, Dw, ea, fG, bB, j, ci, eq, q, ad, wM, dY, fS, aT, Tx, lG, Ds, sp, mQ, g, eE, bE, JM, Tu, m, fD, lC, X, aZN); #c1(CYTH1) c2(NP_004753) c3(cytohesin-1 isoform 1) c4(149311) c5(149312, 149313, 149314, 149315, 149316, 149317, 149318, 149319, 149320, 149321, 149322, 149323) c6( ) c7(hF); #c1(CYTH1) c2(NP_059430) c3(cytohesin-1 isoform 2) c4(193301) c5(193302, 193303, 193304, 193305, 193306, 193307, 193308, 193309, 193310, 193311) c6( ) c7(hF); #c1(CYTH1) c2(XP_006722245) c3(cytohesin-1 isoform X2) c4(294669) c5( ) c6( ) c7(hF); #c1(CYTH1) c2(XP_011523778) c3(cytohesin-1 isoform X3) c4(318270) c5(318271, 318272, 318273, 318274, 318275) c6( ) c7(hF); #c1(CYTH1) c2(XP_011523779) c3(cytohesin-1 isoform X1) c4( ) c5(318276, 318277, 318278, 318279, 318280, 318281, 318282, 318283) c6( ) c7(hF); #c1(CYTH3) c2(NP_004218) c3(cytohesin-3) c4(144385, 144386) c5(144387, 144388, 144389, 144390, 144391, 144392, 144393, 144394, 144395, 144396, 144397, 144398) c6( ) c7(T, jv); #c1(CYTIP) c2(NP_004279) c3(cytohesin-interacting protein) c4(144785, 144786) c5(144787, 144788, 144789, 144790, 144791, 144792, 144793, 144794, 144795, 144796, 144797, 144798) c6( ) c7(T, eM, ik, PV, ci, Kw, fb, V, bz); #c1(CYTL1) c2(NP_061129) c3(cytokine-like protein 1 precursor) c4(199265) c5(199266, 199267, 199268, 199269, 199270, 199271) c6( ) c7(Qu, cm); #c1(CYYR1) c2(NP_443186) c3 (cysteine and tyrosine-rich protein 1 precursor) c4(229754) c5(229755, 229756, 229757, 229758, 229759) c6( ) c7(cm, pX, oe, Kw, bM); #c1(CYYR1) c2(XP_011527751) c3(cysteine and tyrosine-rich protein 1 isoform X1) c4( ) c5(322727) c6( ) c7(cm, pX, oe, Kw, bM); #c1(CYYR1) c2(XP_011527752) c3(cysteine and tyrosine-rich protein 1 isoform X2) c4(322728) c5(322729, 322730, 322731, 322732, 322733, 322734) c6( ) c7(cm, pX, oe, Kw, bM); #c1(D2HGDH) c2(NP_001274178) c3(D-2-hydroxyglutarate dehydrogenase, mitochondrial isoform 2) c4(106124, 106125) c5(106126, 106127, 106128, 106129, 106130, 106131, 106132, 106133, 106134, 106135, 106136, 106137) c6( ) c7(bcU, d, dq, bX, bcV, bcW, rl); #c1(D2HGDH) c2(NP_689996) c3(D-2-hydroxyglutarate dehydrogenase, mitochondrial isoform 1 precursor) c4(244673) c5(244674, 244675, 244676, 244677, 244678, 244679, 244680) c6( ) c7(bcU, d, dq, bX, bcV, bcW, rL); #c1(D2HGDH) c2(XP_011510059) c3(D-2-hydroxyglutarate dehydrogenase, mitochondrial isoform X20) c4( ) c5(300676) c6( ) c7(bcU, d, dq, bX, bcV, bcW, rL); #c1(D2HGDH) c2(XP_011510061) c3(D-2-hydroxyglutarate dehydrogenase, mitochondrial isoform X21) c4(300677) c5(300678, 300679, 300680, 300681, 300682, 300683) c6( ) c7(bcU, d, dq, bX, bcV, bcW, rL); #c1(D2HGDH) c2(XP_011510062) c3(D-2-hydroxyglutarate dehydrogenase, mitochondrial isoform X22) c4(300684) c5(300685, 300686, 300687, 300688, 300689, 300690) c6( ) c7(bcU, d, dq, bX, bcV, bcW, rL); #c1(DAAM1) c2(NP_001257449) c3(disheveled-associated activator of morphogenesis 1 isoform 2) c4(95428) c5( ) c6( ) c7(kD, aW, pc, ac, aT); #c1(DAAM1) c2(NP_055807) c3(disheveled-associated activator of morphogenesis 1 isoform 1) c4(183618, 183619) c5(183620, 183621, 183622, 183623, 183624, 183625, 183626, 183627, 183628, 183629, 183630, 183631, 183632, 183633, 183634, 183635, 183636, 183637, 183638, 183639, 183640, 183641) c6( ) c7(kD, aW, pc, ac, aT); #c1(DAAM2) c2(NP_001188356) c3(disheveled-associated activator of morphogenesis 2 isoform 1) c4(79090) c5(79091, 79092, 79093, 79094, 79095, 79096, 79097, 79098) c6( ) c7(es, Dp, fq, fr); #c1(DAAM2) c2(NP_056160) c3(disheveled-associated activator of morphogenesis 2 isoform 2) c4(186029) c5(186030, 186031, 186032, 186033, 186034, 186035, 186036, 186037, 186038, 186039) c6( ) c7(es, Dp, fq, fr); #c1(DAAM2) c2(XP_006715103) c3(disheveled-associated activator of morphogenesis 2 isoform X2) c4( ) c5(288645, 288646, 288647, 288648) c6( ) c7(es, Dp, fq, fr); #c1(DAAM2) c2(XP_006715109) c3(disheveled-associated activator of morphogenesis 2 isoform X4) c4(288649) c5( ) c6( ) c7(es, Dp, fq, fr); #c1(DAB2) c2(NP_001231800) c3(disabled homolog 2 isoform 2) c4(87528, 87529, 87530, 87531) c5(87532, 87533, 87534, 87535, 87536, 87537, 87538, 87539, 87540, 87541, 87542, 87543, 87544, 87545, 87546, 87547, 87548, 87549, 87550, 87551, 87552, 87553, 87554, 87555, 87556) c6( ) c7(T, aC, id, eR, sw, HM, ht, aw, n, ci, V, ad, av, Bl, aoc, aT, dL, nJ, ae, af, eL, xQ, mR, ZH, CN, CH, aW, ff, fe, en, cl, iV, t, bz, fq, bF, hL, adR, np, m, dy); #c1(DAB2) c2(NP_001334) c3(disabled homolog 2 isoform 1) c4(117001, 117002) c5(117003, 117004, 117005, 117006, 117007, 117008, 117009, 117010, 117011, 117012, 117013) c6( ) c7(T, aC, id, eR, sw, HM, ht, aw, n, ci, V, ad, av, Bl, aoc, aT, dL, nJ, ae, af, eL, xl, mR, ZH, CN, cH, aW, ff, fe, en, cl, iV, t, bz, fq, bF, hL, adR, np, m, dy); #c1(DAB2IP) c2(NP_115941) c3(disabled homolog 2-interacting protein isoform 1) c4(224815, 224816) c5(224817, 224818, 224819, 224820, 224821, 224822, 224823, 224824, 224825, 224826, 224827, 224828) c6( ) c7(T, id, ea, aw, cz, n, j, ad, av, aT, hD, h, dL, K, ae, eL, jf, bf, Y, bz, bm, wx, bt, B); #c1(DAB2IP) c2(XP_005251776) c3(disabled homolog 2-interacting protein isoform X6) c4(271170, 271171) c5(271172, 271173, 271174, 271175, 271176) c6( ) c7(T, id, ea, aw, cz, n, j, ad, av, aT, hD, h, dL, K, ae, eL, jf, bf, Y, bz, bm, wx, bt, B); #c1(DAB2IP) c2(XP_005251778) c3(disabled homolog 2-interacting protein isoform X9) c4(271177, 271178) c5(271179, 271180) c6( ) c7(T, id, ea, aw, cz, n, j, ad, av, aT, hD, h, dL, K, ae, eL, jf, bf, Y, bz, bm, wx, bt, B); #c1(DAB2IP) c2(XP_005251781) c3(disabled homolog 2-interacting protein isoform X10) c4( ) c5(271181) c6( ) c7(T, id, ea, aw, cz, n, j, ad, av, aT, hD, h, dL, K, ae, eL, jf, bf, Y, bz, bm, wx, bt, B); #c1(DAB2IP) c2(XP_011516568) c3(disabled homolog 2-interacting protein isoform X4) c4( ) c5(310015, 310016, 310017, 310018, 310019, 310020) c6( ) c7(T, id, ea, aw, cz, n, j, ad, av, aT, hD, h, dL, K, ae, eL, jf, bf, Y, bz, bm, wx, bt, B); #c1(DAB2IP) c2(XP_011516573) c3(disabled homolog 2-interacting protein isoform X1) c4( ) c5(310021, 310022, 310023, 310024, 310025, 310026, 310027, 310028, 310029, 310030) c6( ) c7(T, id, ea, aw, cz, n, j, ad, av, aT, hD, h, dL, K, ae, eL, jf, bf, Y, bz, bm, wx, bt, B); #c1(DACH1) c2(NP_004383) c3(dachshund homolog 1 isoform c) c4(145978) c5(145979, 145980, 145981, 145982, 145983, 145984, 145985) c6( ) c7(bcX, T, eR, e, aA, aj, fr, ci, J, ad, MS, aT, gs, hP, ae, aW, Mz, t, bz, dr, Ew, eM, zG, m, B); #c1 (DACH1) c2(NP_542937) c3(dachshund homolog 1 isoform a) c4(233107) c5(233108, 233109, 233110, 233111, 233112, 233113) c6( ) c7(bcX, T, eR, e, aA, aj, fr, ci, J, ad, MS, aT, gs, hP, ae, aW, Mz, t, bz, dr, Ew, eM, zG, m, B); #c1(DACH1) c2(NP_542938) c3 (dachshund homolog 1 isoform b) c4(233114) c5(233115, 233116, 233117, 233118, 233119, 233120) c6( ) c7(bcX, T, eR, e, aA, aj, fr, ci, J, ad, MS, aT, gs, hP, ae, aW, Mz, t, bz, dr, Ew, eM, zG, m, B); #c1(DACH1) c2(XP_011533241) c3(dachshund homolog 1 isoform X1) c4(329364, 329365) c5(329366, 329367, 329368, 329369, 329370, 329371, 329372, 329373, 329374, 329375, 329376, 329377) c6( ) c7(bcX, T, eR, e, aA, aj, fr, ci, J, ad, MS, aT, gs, hP, ae, aW, Mz, t, bz, dr, Ew, eM, zG, m, B); #c1(DACH2) c2(NP_001132986) c3(dachshund homolog 2 isoform b) c4(52774) c5(52775, 52776, 52777, 52778, 52779, 52780) c6( ) c7(il); #c1(DACH2) c2(NP_001132987) c3(dachshund homolog 2 isoform c) c4(52781, 52782, 52783) c5(52784, 52785, 52786, 52787, 52788, 52789, 52790, 52791, 52792, 52793, 52794, 52795, 52796, 52797, 52798, 52799, 52800) c6( ) c7(il); #c1(DACH2) c2(NP_444511) c3(dachshund homolog 2 isoform a) c4(230348) c5(230349, 230350, 230351, 230352, 230353, 230354, 230355, 230356, 230357, 230358, 230359) c6( ) c7(il); #c1(DACT1) c2(NP_001072988) c3(dapper homolog 1 isoform 2) c4(35474) c5(35475, 35476, 35477, 35478, 35479, 35480) c6( ) c7(bL, x, T, bE, dO, r, aA, bB, aq, v, aF, ci, k, q, B); #c1(DACT1) c2(NP_057735) c3(dapper homolog 1 isoform 1) c4(192579) c5(192580, 192581, 192582, 192583, 192584, 192585) c6( ) c7(bL, x, T, bE, dO, r, aA, bB, aq, v, aF, ci, k, q, B); #c1(DACT2) c2(NP_001273280) c3(dapper homolog 2 isoform c) c4(104600) c5(104601, 104602, 104603, 104604, 104605, 104606) c6( ) c7(gT, T, bz, aq); #c1(DACT2) c2(NP_999627) c3(dapper homolog 2 isoform a) c4(266085) c5(266086, 266087, 266088, 266089, 266090, 266091) c6( ) c7(gT, T, bz, aq); #c1(DACT3) c2(NP_659493) c3(dapper homolog 3 isoform 1) c4(240214) c5(240215, 240216, 240217, 240218, 240219, 240220) c6( ) c7(kl, nM, r, cj, bz, q); #c1(DACT3) c2(XP_011524802) c3(dapper homolog 3 isoform X1) c4( ) c5(319215) c6( ) c7(kl, nM, r, cj, bz, q); #c1(DAD1) c2(NP_001335) c3(dolichyl- diphosphooligosaccharide--protein glycosyltransferase subunit DAD1) c4(117014) c5(117015, 117016, 117017, 117018, 117019, 117020) c6( ) c7(cF, pf, aW, pX, aT, L); #c1(DAG1) c2(NP_001171113) c3(dystroglycan preproprotein) c4(69823, 69824, 69825, 69826, 69827, 69828, 69829, 69830, 69831) c5(69832, 69833, 69834, 69835, 69836, 69837, 69838, 69839, 69840, 69841, 69842, 69843, 69844, 69845, 69846, 69847, 69848, 69849, 69850, 69851, 69852, 69853, 69854, 69855, 69856, 69857, 69858, 69859, 69860, 69861, 69862, 69863, 69864, 69865, 69866, 69867, 69868, 69869, 69870, 69871, 69872, 69873, 69874, 69875, 69876, 69877, 69878, 69879, 69880, 69881, 69882, 69883, 69884, 69885, 69886, 69887, 69888, 69889, 69890, 69891, 69892, 69893, 69894, 69895, 69896, 69897, 69898, 69899, 69900, 69901, 69902, 69903, 69904, 69905) c6( ) c7(bda, bM, T, dD, ea, cz, bB, n, v, eq, ci, nA, bX, EA, fr, cd, ad, cc, no, sN, Ae, Dq, oj, aT, Ak, bK, K, bcY, cb, Wq, ae, bc, jf, VC, aqZ, aW, ff, xs, FD, fQ, uY, en, g, alj, es, Bv, iA, dB, bE, go, FB, mx, km, uJ, bcZ, IM, X, lf); #c1(DAND5) c2(NP_689867) c3(DAN domain family member 5 precursor) c4(244213) c5(244214, 244215, 244216, 244217, 244218, 244219, 244220, 244221, 244222) c6(244223, 244224) c7(ck, bM, T, bv, aE, eM, nO, Y, r, hD, uz, vx, bz, q); #c1(DAD) c2(XP_011536307) c3(D-amino-acid oxidase isoform X1) c4(333648, 333649) c5(333650, 333651, 333652, 333653, 333654, 333655, 333656, 333657, 333658, 333659, 333660, 333661) c6( ) c7(mU, hq, iH, rz, mW, Fo, mZ, agb, mS, gl, mY, bK, K, eA, oi, mV, mT, bN, mX); #c1(DADA) c2(NP_001155284) c3(D-amino acid oxidase activator isoform 2) c4(61569) c5(61570, 61571, 61572, 61573, 61574, 61575) c6( ) c7(b0, mS, ww, ct, dY, mW, aUb, bK, oi, mY, mV, he, af, rz, mU, agb, gl, Eg, Z, jb, mZ, bN, mX, bdc, W, ac, cA, sv, bdb, mT); #c1(DADA) c2(NP_001155286) c3(D-amino acid oxidase activator isoform 3) c4( ) c5(61576, 61577, 61578, 61579, 61580) c6( ) c7(b0, mS, ww, ct, dY, mW, aUb, bK, oi, mY, mV, he, af, rz, mU, agb, gl, Eg, Z, jb, mZ, bN, mX, bdc, W, ac, cA, sv, bdb, mT); #c1(DADA) c2(NP_758958) c3(D-amino acid oxidase activator isoform 1) c4(249405, 249406) c5(249407, 249408, 249409, 249410, 249411, 249412, 249413) c6( ) c7(b0, mS, ww, ct, dY, mW, aUb, bK, oi, mY, mV, he, af, rz, mu, agb, gl, Eg, Z, jb, mZ, bN, mX, bdc, W, ac, cA, sv, bdb, mT); #c1(DAP) c2(NP_001278892) c3(death-associated protein 1 isoform 1) c4( ) c5(110835) c6( ) c7(T, dD, fM, eP, pf, ea, if, ha, ht, fd, aA, Bm, n, aj, vm, eb, ci, eq, qN, il, q, eA, XX, av, pe, hW, Vr, aT, dL, hu, eL, je, aW, ff, r, ks, aF, jh, apm, en, g, t, cm, bz, ik, iV, Wt, ck, qJ, bF, amS, nM, iM, eM, AM, vA, gX, cj, hR, m, adg, bt, aln); #c1(DAP) c2(NP_004385) c3(death-associated protein 1 isoform 2) c4(145986) c5(145987, 145988, 145989, 145990, 145991) c6( ) c7(T, dD, fM, eP, pf, ea, if, ha, ht, fd, aA, Bm, n, aj, vm, eb, ci, eq, qN, il, q, eA, XX, av, pe, hW, Vr, aT, dl, hu, eL, je, aW, ff, r, ks, aF, jh, apm, en, g, t, cm, bz, ik, iV, Wt, ck, qJ, bF, amS, nM, iM, eM, AM, vA, gX, cj, hR, m, adg, bt, aln); #c1(DAP3) c2(NP_001186778) c3(28S ribosomal protein S29, mitochondrial isoform 1) c4(78554) c5(78555, 78556, 78557, 78558, 78559, 78560, 78561, 78562, 78563, 78564) c6( ) c7(bM, d, nB, aA, aT, aF, pd, eA, ad); #c1(DAP3) c2(XP_005245537) c3(28S ribosomal protein S29, mitochondrial isoform X1) c4(266704) c5(266705, 266706, 266707, 266708, 266709, 266710) c6( ) c7(bM, d, nB, aA, aT, aF, pd, eA, ad); #c1(DAP3) c2(XP_005245538) c3(28S ribosomal protein S29, mitochondrial isoform X2) c4(266711) c5(266712, 266713, 266714, 266715, 266716, 266717) c6( ) c7(bM, d, nB, aA, aT, aF, pd, eA, ad); #c1(DAPK1) c2(NP_004929) c3(death-associated protein kinase 1) c4(151008, 151009) c5(151010, 151011, 151012, 151013, 151014, 151015, 151016, 151017, 151018, 151019, 151020, 151021, 151022, 151023, 151024, 151025, 151026, 151027, 151028, 151029, 151030, 151031, 151032, 151033, 151034, 151035) c6( ) c7(gV, T, id, wu, atj, if, qx, ht, aA, n, fr, ci, bX, qN, il, fw, eA, iC, av, Bl, jz, py, x, cF, qK, aT, dL, bde, K, wm, hu, eL, af, il, jf, qw, bdd, d, BN, iU, hG, JI, aW, Y, ff, eH, r, aF, en, g, eF, bw, ik, bz, op, ck, es, bb, Dy, eM, iA, aJt, dB, AM, vA, bdf, cj, pg, m, bt, hS); #c1 (DAPK1) c2(XP_005251814) c3(death-associated protein kinase 1 isoform X1) c4(271199) c5( ) c6( ) c7(gV, T, id, wu, atj, if, qx, ht, aA, n, fr, ci, bX, qN, il, fw, eA, iC, av, Bl, jz, py, x, cF, qK, aT, dL, bde, K, wm, hu, eL, af, iL, jf, qw, bdd, d, BN, iU, hG, JI, aW, Y, ff, eH, r, aF, en, g, eF, bw, ik, bz, op, ck, es, bb, Dy, eM, iA, alt, dB, AM, vA, bdf, cj, pg, m, bt, hS); #c1(DAPK2) c2(NP_055141) c3(death-associated protein kinase 2) c4(179389) c5(179390, 179391, 179392, 179393, 179394, 179395, 179396, 179397, 179398, 179399) c6( ) c7(ID, ly, wg, c, Ae, aW, U, aT, aA, cy, fd, dG, dL, ci, K, an, jj, bt, lx); #c1(DAPK2) c2(XP_011519716) c3(death-associated protein kinase 2 isoform X3) c4(314378) c5( ) c6( ) c7(ID, ly, wg, c, Ae, aW, U, aT, aA, cy, fd, dG, dl, ci, K, an, jj, bt, lx); #c1(DAPK2) c2(XP_011519720) c3(death-associated protein kinase 2 isoform X7) c4( ) c5(314379, 314380, 314381, 314382, 314383, 314384) c6( ) c7(ID, ly, wg, c, Ae, aW, U, aT, aA, cy, fd, dG, dL, ci, K, an, jj, bt, lx); #c1(DAPK3) c2(XP_005259565) c3(death-associated protein kinase 3 isoform X1) c4(276126, 276127) c5(276128, 276129, 276130, 276131, 276132, 276133, 276134, 276135, 276136, 276137, 276138, 276139, 276140, 276141, 276142, 276143) c6( ) c7(T, aC, fM, Tb, e, cz, aj, zx, ci, pz, J, ad, aE, yw, aT, K, ae, kj, iU, aW, cm, bw, bz, iO, eM, dh, B); #c1(DARS) c2(NP_001280241) c3(aspartate-tRNA ligase, cytoplasmic isoform 2) c4( ) c5(111518, 111519, 111520, 111521, 111522) c6( ) c7(bdh, cm, bdg); #c1(DARS) c2(NP_001340) c3(aspartate-tRNA ligase, cytoplasmic isoform 1) c4(117035, 117036) c5(117037, 117038, 117039, 117040, 117041, 117042, 117043) c6( ) c7(bdh, cm, bdg); #c1(DARS2) c2(NP_060592) c3(aspartate-tRNA ligase, mitochondrial) c4(196719, 196720) c5(196721, 196722, 196723, 196724, 196725, 196726, 196727, 196728, 196729, 196730, 196731, 196732) c6( ) c7(bL, W, bdh, bdi, akr, V); #c1(DAW1) c2(NP_849143) c3(dynein assembly factor with WDR repeat domains 1) c4(254248, 254249) c5(254250, 254251, 254252, 254253, 254254, 254255, 254256) c6( ) c7(oe, an); #c1(DAW1) c2(XP_011509057) c3(dynein assembly factor with WDR repeat domains 1 isoform X1) c4( ) c5(299666, 299667, 299668, 299669, 299670) c6( ) c7(oe, an); #c1(DAXX) c2(NP_001135442) c3(death domain-associated protein 6 isoform b) c4( ) c5(52909, 52910, 52911, 52912, 52913, 52914, 52915, 52916, 52917, 52918, 52919, 52920, 52921, 52922, 52923, 52924, 52925, 52926, 52927, 52928, 52929, 52930, 52931, 52932, 52933, 52934, 52935) c6( ) c7(T, aC, fR, ci, pX, BJ, iv, K, bdj, jj, pi, A, asP, ev, CM, eZ, lo, vs, bdk, t, Hv, ck, AH, k, aan, m, y, bt, KS, st); #c1(DAXX) c2(NP_001241646) c3(death domain-associated protein 6 isoform c) c4(88944) c5( ) c6( ) c7(T, aC, fR, ci, pX, BJ, iv, K, bdj, jj, pi, A, asP, ev, CM, eZ, lo, vs, bdk, t, Hv, ck, AH, k, aan, m, y, bt, kS, st); #c1(DAXX) c2(NP_001341) c3(death domain-associated protein 6 isoform a) c4(117044, 117045, 117046, 117047) c5(117048, 117049, 117050, 117051, 117052, 117053, 117054, 117055, 117056, 117057, 117058, 117059, 117060, 117061, 117062, 117063, 117064, 117065, 117066, 117067, 117068, 117069, 117070, 117071, 117072, 117073, 117074, 117075, 117076, 117077, 117078, 117079, 117080, 17081, 117082, 117083, 117084, 117085, 117086, 117087, 117088, 117089, 117090, 117091, 117092, 117093, 117094, 117095, 117096, 117097, 117098, 117099, 117100, 117101, 117102, 117103, 117104, 117105, 117106, 117107, 117108, 117109, 117110, 117111, 117112, 117113, 117114, 117115, 117116, 117117, 117118, 117119, 117120, 117121, 117122, 117123, 117124, 117125, 117126, 117127, 117128, 117129, 117130, 117131, 117132, 117133, 117134) c6( ) c7(T, aC, fR, ci, pX, BJ, iv, K, bdj, jj, pi, A, asP, ev, CM, eZ, lo, vs, bdk, t, Hv, ck, AH, k, aan, m, y, bt, kS, st); #c1(DAZ1) c2(NP_004072) c3(deleted in azoospermia protein 1) c4(143310, 143311, 143312, 143313) c5(143314, 143315, 143316, 143317, 143318, 143319, 143320, 143321, 143322, 143323, 143324, 143325) c6( ) c7(yi, gU, ND, vJ, vH, UI, bX, bdl, on, KL); #c1(DAZ2) c2(NP_001005785) c3(deleted in azoospermia protein 2 isoform 2) c4(22078, 22079) c5(22080, 22081, 22082, 22083, 22084, 22085, 22086, 22087, 22088, 22089, 22090, 22091, 22092, 22093, 22094, 22095, 22096) c6( ) c7(gU, UI, ND); #c1(DAZ2) c2(NP_001005786) c3(deleted in azoospermia protein 2 isoform 3) c4(22097, 22098, 22099) c5(22100, 22101, 22102, 22103, 22104, 22105, 22106, 22107, 22108, 22109, 22110, 22111, 22112, 22113, 22114, 22115) c6( ) c7(gU, UI, ND); #c1(DAZ2) c2(NP_065096) c3(deleted in azoospermia protein 2 isoform 1) c4(203876, 203877) c5(203878, 203879, 203880, 203881, 203882, 203883, 203884, 203885, 203886, 203887, 203888, 203889) c6( ) c7(gU, UI, ND); #c1(DAZ3) c2(NP_065097) c3(deleted in azoospermia protein 3) c4(203890, 203891, 203892) c5(203893, 203894, 203895, 203896, 203897, 203898, 203899, 203900, 203901, 203902, 203903, 203904, 203905, 203906, 203907, 203908, 203909, 203910, 203911, 203912, 203913, 203914, 203915, 203916, 203917, 203918, 203919, 203920, 203921, 203922, 203923, 203924, 203925) c6( ) c7(ND, bX); #c1(DAZ4) c2(NP_001005375) c3(deleted in azoospermia protein 4 isoform 1) c4(21579) c5(21580, 21581, 21582, 21583, 21584, 21585, 21586, 21587, 21588) c6( ) c7(ND, gU, bX); #c1(DAZ4) c2(NP_065153) c3(deleted in azoospermia protein 4 isoform 2) c4(204269, 204270, 204271, 204272, 204273) c5(204274, 204275, 204276, 204277, 204278, 204279, 204280, 204281, 204282, 204283, 204284, 204285, 204286, 204287, 204288, 204289, 204290, 204291, 204292, 204293, 204294, 204295, 204296, 204297, 204298, 204299, 204300, 204301, 204302, 204303, 204304, 204305, 204306, 204307, 204308, 204309, 204310, 204311, 204312, 204313, 204314, 204315, 204316, 204317, 204318, 204319, 204320, 204321, 204322, 204323, 204324, 204325, 204326) c6( ) c7(ND, gU, bX); #c1(DAZ4) c2(XP_005262659) c3 (deleted in azoospermia protein 4 isoform X1) c4(278028, 278029, 278030) c5( ) c6( ) c7(ND, gU, bX); #c1(DAZ4) c2(XP_011529811) c3(deleted in azoospermia protein 4 isoform X2) c4( ) c5(325579, 325580, 325581, 325582, 325583, 325584, 325585, 325586, 325587, 325588, 325589, 325590, 325591) c6( ) c7(ND, gU, bX); #c1(DAZAP1) c2(NP_061832) c3(DAZ-associated protein 1 isoform b) c4(200681) c5(200682, 200683, 200684, 200685, 200686, 200687) c6( ) c7(x, W, T, k); #c1(DAZAP1) c2(NP_733829) c3(DAZ-associated protein 1 isoform a) c4(247211) c5(247212, 247213, 247214, 247215, 247216, 247217) c6( ) c7(x, W, T, k); #c1(DAZAP1) c2(XP_011526212) c3(DAZ-associated protein 1 isoform X7) c4( ) c5(320477) c6( ) c7(x, W, T, k); #c1(DAZAP2) c2(NP_001129736) c3(DAZ-associated protein 2 isoform b) c4(52271) c5(52272, 52273, 52274, 52275, 52276) c6( ) c7(fd); #c1(DAZAP2) c2(NP_001129738) c3(DAZ-associated protein 2 isoform c) c4(52277) c5(52278, 52279, 52280, 52281, 52282, 52283, 52284, 52285, 52286, 52287) c6( ) c7(fd); #c1(DAZAP2) c2(NP_001129739) c3(DAZ-associated protein 2 isoform d) c4(52288) c5(52289, 52290, 52291, 52292, 52293) c6( ) c7(fd); #c1(DAZAP2) c2(NP_001129740) c3(DAZ-associated protein 2 isoform e) c4(52294) c5(52295, 52296, 52297, 52298, 52299) c6( ) c7(fd); #c1(DAZAP2) c2(NP_001129741) c3(DAZ-associated protein 2 isoform f) c4(52300) c5(52301, 52302, 52303, 52304, 52305, 52306) c6( ) c7(fd); #c1(DAZAP2) c2(NP_055579) c3(DAZ-associated protein 2 isoform a) c4(182063) c5(182064, 182065, 182066, 182067, 182068) c6( ) c7(fd); #c1(DAZL) c2(NP_001177740) c3(deleted in azoospermia-like isoform 1) c4(72954) c5(72955, 72956, 72957, 72958, 72959, 72960) c6( ) c7(zZ, gU, ND, il, Lf, vH, bu, pt, Ld); #c1(DAZL) c2(NP_001342) c3(deleted in azoospermia-like isoform 2) c4(117135) c5(1/7/36, 117137, 117138, 117139, 117140, 117141) c6( ) c7(zZ, gU, ND, il, Lf, vH, bu, pt, Ld); #c1(DBF4) c2(NP_006707) c3(protein DBF4 homolog A) c4(167312) c5(167313, 167314, 167315, 167316, 167317, 167318) c6( ) c7(Dy, Y, K); #c1(DBF4) c2(XP_005250177) c3(protein DBF4 homolog A isoform X1) c4(270166) c5(270167) c6( ) c7(Dy, Y, K); #c1(DBF4) c2(XP_005250179) c3(protein DBF4 homolog A isoform X5) c4( ) c5(270168, 270169, 270170, 270171, 270172) c6( ) c7(Dy, Y, K); #c1(DBH) c2(NP_000778) c3(dopamine beta-hydroxylase precursor) c4(15633, 15634, 15635) c5(15636, 15637, 15638, 15639, 15640, 15641, 15642, 15643, 15644, 15645, 15646, 15647, 15648, 15649, 15650) c6( ) c7(bdo, hl, aaM, rG, wJ, e, ha, uG, b, cz, GC, ti, bS, ur, bdm, ct, uu, bdr, ml, V, bX, cd, aR, xt, VV, HF, hb, sO, Qg, Fo, ep, aU, aU, Sb, bdp, gl, K, ama, bq, bK, mY, he, an, af, eu, na, Wm, ak, Wi, Y, yL, Gv, fQ, bds, HL, Eg, Z, FX, PF, cm, dr, GB, hq, qa, bN, bdn, pH, ac, cA, Ei, U, cj, Rm, Km, dy, bdq, xg); #c1(DBI) c2(NP_001073331) c3(acyl-CoA-binding protein isoform 3) c4(35871) c5(35872, 35873, 35874, 35875, 35876, 35877) c6( ) c7(bL, T, zQ, aPf, Fb, ci, K, abl, zP, ae, ad, hq); #c1(DBI) c2(NP_001073332) c3(acyl-CoA-binding protein isoform 2) c4(35878) c5(35879, 35880, 35881, 35882, 35883, 35884) c6( ) c7(bL, T, zQ, aPf, Fb, ci, K, abd, zP, ae, ad, hq); #c1(DBI) c2(NP_001171488) c3(acyl-CoA-binding protein isoform 4) c4(70312) c5(70313, 70314, 70315, 70316, 70317, 70318) c6( ) c7(bL, T, zQ, aPf, Fb, ci, K, abQ, zP, ae, ad, hq); #c1(DBI) c2(NP_001171512) c3(acyl-CoA-binding protein isoform 5) c4(70384) c5(70385, 70386, 70387, 70388, 70389, 70390) c6( ) c7(bL, T, zQ, aPf, Fb, ci, K, abQ, zP, ae, ad, hq); #c1(DBI) c2(NP_001171514) c3(acyl-CoA-binding protein isoform 6) c4(70391) c5( ) c6( ) c7(bL, T, zQ, aPf, Fb, ci, K, abl, zP, ae, ad, hq); #c1(DBI) c2(NP_065438) c3(acyl-CoA-binding protein isoform 1) c4(204791) c5(204792, 204793, 204794, 204795, 204796, 204797, 204798, 204799, 204800, 204801, 204802, 204803, 204804, 204805) c6( ) c7(bL, T, zQ, aPf, Fb, ci, K, abQ, zP, ae, ad, hq); #c1(DBN1) c2(NP_004386) c3(drebrin isoform a) c4(145992, 145993, 145994) c5(145995, 145996, 145997, 145998, 145999, 146000, 146001, 146002, 146003, 146004, 146005, 146006, 146007, 146008, 146009, 146010, 146011, 146012, 146013) c6( ) c7(d, af, eM, U, bK, eA, nA, xg, cd); #c1(DBN1) c2(NP_543157) c3(drebrin isoform b) c4(233479) c5(233480, 233481, 233482, 233483, 233484, 233485) c6( ) c7(d, af, eM, U, bK, eA, nA, xg, cd); #c1(DBN1) c2(XP_005265884) c3(drebrin isoform X2) c4(279818) c5( ) c6( ) c7(d, af, eM, U, bK, eA, nA, xg, cd); #c1(DBN1) c2(XP_005265886) c3(drebrin isoform X6) c4( ) c5(279819, 279820, 279821, 279822, 279823) c6( ) c7(d, af, eM, U, bK, eA, nA, xg, cd); #c1(DBNL) c2(NP_001014436) c3(drebrin-like protein isoform b) c4(25880, 25881, 25882, 25883) c5(25884, 25885, 25886, 25887, 25888, 25889, 25890, 25891, 25892, 25893, 25894, 25895, 25896, 25897, 25898, 25899, 25900, 25901, 25902) c6( ) c7(Nq, bM, cT, Nr, JM, aDB, aj, Xm, bK, xl, aLY); #c1(DBNL) c2(NP_001116428) c3(drebrin-like protein isoform c) c4(43870) c5(43871, 43872, 43873, 43874, 43875, 43876) c6( ) c7(Nq, bM, cT, Nr, JM, aOB, aj, Xm, bK, xl, aLY); #c1(DBNL) c2(NP_001271242) c3(drebrin-like protein isoform d) c4( ) c5(103719, 103720, 103721, 103722, 103723) c6( ) c7(Nq, bM, cT, Nr, JM, aOB, aj, Xm, bK, xl, aLY); #c1(DBNL) c2(NP_001271244) c3(drebrin-like protein isoform e) c4(103724) c5(103725, 103726, 103727, 103728, 103729, 103730) c6( ) c7(Nq, bM, cT, Nr, JM, aOB, aj, Xm, bK, xl, aLY); #c1(DBNL) c2(NP_054782) c3(drebrin-like protein isoform a) c4(177797, 177798, 177799) c5(177800, 177801, 177802, 177803, 177804, 177805, 177806, 177807, 177808, 177809, 177810, 177811, 177812, 177813, 177814, 177815, 177816, 177817) c6( ) c7(Nq, bM, cT, Nr, JM, aOB, aj, Xm, bK, xl, aLY); #c1(DBP) c2(NP_001343) c3(D site-binding protein) c4(117142) c5(117143, 117144, 117145, 117146, 117147, 117148, 117149) c6( ) c7(bL, am, eR, jh, cz, aj, dG, v, il, V, qW, ai, J, eA, eW, xy, aT, hD, afV, dL, bK, K, eL, bA, A, mR, Pd, Z, hq, yE, at, pk); #c1(DBR1) c2(NP_057300) c3(lariat debranching enzyme) c4(189964, 189965) c5(189966, 189967, 189968, 189969, 189970, 189971, 189972, 189973, 189974, 189975, 189976, 189977) c6( ) c7(h, ql, c, zn); #c1(DBX1) c2(NP_001025036) c3(homeobox protein DBX1) c4(29246, 29247) c5(29248, 29249, 29250, 29251, 29252, 29253, 29254, 29255, 29256, 29257, 29258, 29259) c6( ) c7(PJ); #c1(DCAF12) c2(NP_056212) c3(DDB1- and CUL4-associated factor (2) c4(186406, 186407) c5(186408, 186409, 186410, 186411, 186412, 186413, 186414, 186415, 186416, 186417, 186418, 186419) c6( ) c7(ae, T, bz, ad, bu); #c1(DCAF12) c2(XP_006716815) c3(DDB1- and CUL4-associated factor 12 isoform X1) c4(290496) c5(290497, 290498, 290499, 290500, 290501, 290502) c6( ) c7(ae, T, bz, ad, bu); #c1(DCAF13) c2(NP_056235) c3(DDB1- and CUL4-associated factor 13) c4(186639, 186640) c5(186641, 186642, 186643, 186644, 186645, 186646, 186647, 186648, 186649, 186650, 186651, 186652) c6( ) c7(bA); #c1(DCAF17) c2(NP_001158293) c3(DDB1- and CUL4-associated factor 17 isoform 2) c4(63565) c5(63566, 63567, 63568, 63569, 63570, 63571) c6( ) c7(bdt, aU, aeM, aj, nA, UE); #c1(DCAF17) c2(NP_079276) c3(DDB1- and CUL4-associated factor 17 isoform 1) c4(217658, 217659) c5(217660, 217661, 217662, 217663, 217664, 217665) c6( ) c7(bdt, aU, aeM, aj, nA, UE); #c1(DCAF17) c2(XP_006712836) c3(DDB1- and CUL4-associated factor 17 isoform X7) c4( ) c5(286885, 286886, 286887, 286888, 286889, 286890) c6( ) c7(bdt, aU, aeM, aj, nA, UE); #c1(DCAF4) c2(NP_001156980) c3(DDB1- and CUL4-associated factor 4 isoform 4) c4(62047) c5(62048, 62049, 62050, 62051, 62052, 62053) c6( ) c7(bM); #c1(DCAF4) c2(NP_056419) c3(DDB1- and CUL4-associated factor 4 isoform 1) c4(187524) c5(187525, 187526, 187527, 187528, 187529, 187530) c6( ) c7(bM); #c1(DCAF4) c2(NP_851937) c3(DDB1- and CUL4-associated factor 4 isoform 2) c4(254701) c5(254702, 254703, 254704, 254705, 254706) c6( ) c7(bM); #c1(DCAF4) c2(NP_851938) c3(DDB1- and CUL4-associated factor 4 isoform 3) c4(254707) c5(254708, 254709, 254710, 254711, 254712, 254713) c6( ) c7(bM); #c1(DCAF4) c2(XP_005267581) c3(DDB1- and CUL4-associated factor 4 isoform X3) c4( ) c5(281023) c6( ) c7(bM); #c1(DCAF5) c2(NP_001271136) c3(DDB1- and CUL4-associated factor 5 isoform 3) c4(103438) c5(103439, 103440, 103441, 103442, 103443, 103444, 103445, 103446) c6( ) c7(j); #c1(DCAF5) c2(NP_001271137) c3(DDB1- and CUL4-associated factor 5 isoform 4) c4( ) c5(103447, 103448, 103449, 103450, 103451, 103452) c6( ) c7(j); #c1(DCAF5) c2(NP_003852) c3(DDB1- and CUL4-associated factor 5 isoform 1) c4(141418) c5(141419, 141420, 141421, 141422, 141423, 141424, 141425, 141426) c6( ) c7(j); #c1(DCAF5) c2(XP_006720363) c3(DDB1- and CUL4-associated factor 5 isoform X4) c4(293503) c5( ) c6( ) c7(j); #c1(DCAF6) c2(NP_001017977) c3(DDB1- and CUL4-associated factor 6 isoform b) c4(26703) c5(26704, 26705, 26706, 26707, 26708, 26709) c6( ) c7(bA, ae, dA, ad, cz); #c1(DCAF6) c2(NP_001185885) c3(DDB1- and CUL4-associated factor 6 isoform c) c4(76650) c5(76651, 76652, 76653, 76654, 76655, 76656) c6( ) c7(bA, ae, dA, ad, cz); #c1(DCAF6) c2(NP_001185886) c3(DDB1- and CUL4-associated factor 6 isoform d) c4(76657) c5(76658, 76659, 76660, 76661, 76662, 76663) c6( ) c7(bA, ae, dA, ad, cz); #c1(DCAF6) c2(NP_060912) c3(DDB1- and CUL4-associated factor 6 isoform a) c4(198594) c5(198595, 198596, 198597, 198598, 198599, 198600) c6( ) c7(bA, ae, dA, ad, cz); #c1(DCAF6) c2(XP_011508069) c3(DDB1- and CUL4-associated factor 6 isoform X4) c4( ) c5(298534, 298535, 298536) c6( ) c7(bA, ae, dA, ad, cz); #c1(DCAF7) c2(NP_005819) c3(DDB1- and CUL4-associated factor 7) c4(159660, 159661, 159662) c5(159663, 159664, 159665, 159666, 159667, 159668, 159669, 159670, 159671, 159672, 159673, 159674, 159675, 159676, 159677, 159678, 159679, 159680) c6( ) c7(T, qn, fd, lR, Qp, aY, Tz, x, bv, fE, dV, aU, eL, Us, azh, KG, apl, aW, Az, ace, g, eF, aHd, mZ, bdx, eM, Kv, aDs, nw, aYS, k, m, LT, WA, RC, n, aj, fr, nA, bd, nE, rc, Nh, Oe, ft, afn, acA, adX, h, aDa, dL, aUH, mY, mV, dx, Lg, mU, yi, A, Cw, aJ, aVf, zb, agL, AQ, agG, ack, QK, mX, hq, ej, adZ, dB, mg, zG, bdA, bD, B, mT, BC, fM, eR, aYJ, aA, cz, mS, v, C, aqf, bdB, aYh, akW, J, hb, aT, kf, bK, zp, ae, bA, bdy, tN, sb, arl, bdz, Qr, iU, axW, Y, bdv, t, f, iK, pb, cT, pa, apq, ac, Zg, mf, rK, bt, ps, yv, gV, ha, jh, wV, ad, hX, iC, db, vJ, mW, oj, oW, agm, fb, bdw, atJ, yr, d, hG, et, xs, eH, EZ, avG, ayj, aU, bdu, ek, pv, EW, DT, Ew, kq, azM, awK, y, hv); #c1(DCAF8) c2(NP_056541) c3(DDB1- and CUL4-associated factor 8) c4(188022, 188023, 188024, 188025, 188026, 188027, 188028) c5(188029, 188030, 188031, 188032, 188033, 188034, 188035, 188036, 188037, 188038, 188039, 188040, 188041, 188042, 188043, 188044, 188045, 188046, 188047, 188048, 188049, 188050, 188051, 188052, 188053, 188054, 188055, 188056, 188057, 188058, 188059, 188060, 188061, 188062, 188063, 188064, 188065, 188066, 188067, 188068, 188069, 188070, 188071, 188072, 188073, 188074, 188075, 188076, 188077, 188078, 188079, 188080, 188081) c6( ) c7(cc, ql, bdC); #c1(DCANP1) c2(NP_570900) c3(dendritic cell nuclear protein 1) c4(234393) c5(234394, 234395, 234396, 234397, 234398, 234399, 234400) c6( ) c7(bM, eE, bO, bS, K, ct, Z); #c1(DCBLD1) c2(NP_775945) c3(discoidin, CUB and LCCL domain-containing protein 1 precursor) c4(250762) c5(250763, 250764, 250765, 250766, 250767, 250768, 250769, 250770, 250771, 250772) c6( ) c7(Y, cd, dY); #c1(DCBLD1) c2(XP_005266998) c3(discoidin, CUB and LCCL domain-containing protein 1 isoform X1) c4(280603) c5(280604, 280605, 280606, 280607, 280608, 280609) c6( ) c7(Y, cd, dY); #c1(DCBLD1) c2(XP_011534074) c3(discoidin, CUB and LCCL domain-containing protein 1 isoform X3) c4(330256) c5(330257, 330258, 330259, 330260, 330261) c6( ) c7(Y, cd, dY); #c1(DCBLD1) c2(XP_011534075) c3(discoidin, CUB and LCCL domain-containing protein 1 isoform X4) c4(330262) c5(330263, 330264) c6( ) c7(Y, cd, dY); #c1(DCBLD1) c2(XP_011534076) c3(discoidin, CUB and LCCL domain-containing protein 1 isoform X5) c4( ) c5(330265) c6( ) c7(Y, cd, dY); #c1(DCBLD2) c2(NP_563615) c3(discoidin, CUB and LCCL domain-containing protein 2 precursor) c4(233540, 233541, 233542) c5(233543, 233544, 233545, 233546, 233547, 233548, 233549, 233550, 233551, 233552, 233553, 233554, 233555, 233556, 233557, 233558, 233559, 233560) c6( ) c7(bM, T, kl, d, ant, aDy, oV, ea, EQ, la, aA, aF, bz); #c1(DCC) c2(NP_005206) c3(netrin receptor DCC) c4(153986, 153987) c5(153988, 153989, 153990, 153991, 153992, 153993) c6( ) c7(T, aC, e, fd, bB, ci, ip, x, aE, eL, aW, nY, g, ir, eF, cm, bw, eM, dc, m, YK, Qj, aw, ht, NT, n, aj, h, lG, hn, dl, hB, bdD, fe, YF, W, dB, hL, it, B, aA, j, q, er, eA, eJ, cF, hW, aT, Di, an, ae, eu, s, vT, NE, iU, Y, r, qL, pR, en, cl, t, iK, ck, ac, gX, bt, id, jh, bS, ad, Dq, io, hD, d, ff, eH, aF, iV, bz, bF, bE, y, X); #c1(DCC) c2(XP_011524148) c3(netrin receptor DCC isoform X4) c4(318704) c5(318705, 318706, 318707, 318708, 318709, 318710, 318711, 318712, 318713, 318714, 318715, 318716) c6( ) c7(T, aC, e, fd, bB, ci, ip, x, aE, eL, aW, nY, g, ir, eF, cm, bw, eM, dc, m, YK, Qj, aw, ht, NT, n, aj, h, lG, hn, dL, hB, bdD, fe, YF, W, dB, hL, it, B, aA, j, q, er, eA, eJ, cF, hW, aT, Oi, an, ae, eu, s, vT, NE, iU, Y, r, qL, pR, en, cl, t, iK, ck, ac, gX, bt, id, jh, bS, ad, Dq, io, hD, d, ff, eH, aF, iV, bz, bF, bE, y, X); #c1(DCD) c2(NP_444513) c3(dermcidin isoform 1 preproprotein) c4(230360, 230361) c5(230362, 230363, 230364, 230365, 230366, 230367) c6( ) c7(bL, T, bdE, aE, fR, aW, Y, aT, tc, aq, oH, K, lQ, KO, eE, ae, B, ad, bA); #c1(DCDC2) c2(NP_057440) c3(doublecortin domain-containing protein 2) c4(190833, 190834, 190835, 190836) c5(190837, 190838, 190839, 190840, 190841, 190842, 190843, 190844, 190845, 190846, 190847, 190848, 190849, 190850, 190851, 190852, 190853, 190854, 190855, 190856, 190857, 190858, 190859, 190860, 190861) c6( ) c7(ae, T, ac, VT, aaw, yA, alP, afx, bdF, B, xt, ad); #c1(DCDC2C) c2(NP_001274373) c3(doublecortin domain-containing protein 2C) c4( ) c5(106375, 106376, 106377, 106378, 106379, 106380) c6( ) c7(bL, bM, aC, oe, j, an, fq, cd); #c1(DCDCZC) c2(XP_006711957) c3(doublecortin domain-containing protein 2C isoform X5) c4(286307) c5( ) c6( ) c7(bL, bM, aC, oe, j, an, fq, cd); #c1(DCHS1) c2(NP_003728) c3(protocadherin-16 precursor) c4(140479) c5(140480, 140481, 140482, 140483, 140484, 140485, 140486) c6( ) c7(aur, afw, bdG, j); #c1(DCHS2) c2(NP_001136024) c3(protocadherin-23 isoform 2) c4(54037, 54038) c5(54039, 54040, 54041, 54042, 54043, 54044, 54045, 54046, 54047, 54048, 54049, 54050) c6( ) c7(V, oe, af); #c1(DCHS2) c2(NP_060109) c3(protocadherin-23 isoform 1) c4(194220) c5(194221, 194222, 194223, 194224, 194225, 194226) c6( ) c7(V, De, af); #c1(DCHS2) c2(XP_011530347) c3(protocadherin-23 isoform X1) c4(326206, 326207) c5(326208, 326209, 326210, 326211, 326212, 326213, 326214, 326215, 326216, 326217, 326218, 326219) c6( ) c7(V, oe, af); #c1(DCK) c2(NP_000779) c3(deoxycytidine kinase) c4(15651, 15652) c5(15653, 15654, 15655, 15656, 15657, 15658, 15659, 15660, 15661, 15662, 15663, 15664) c6( ) c7(T, aC, iW, ht, jh, ci, bX, BA, x, aE, nv, aT, h, DU, hS, aW, kp, hf, t, f, cm, bw, ck, nM, eM, bE, zv, m, k, bt, iu, ix, On); #c1(DCLK1) c2(NP_001182344) c3(serine/threonine-protein kinase DCLK1 isoform 2) c4( ) c5(74804, 74805, 74806, 74807, 74808, 74809) c6( ) c7(HQ, T, ac, Yc, s, dH, aE, e, r, aq, kp, ey, q, eA, cm, B); #c1(DCLK1) c2(NP_001182345) c3(serine/threonine-protein kinase DCLK1 isoform 3) c4(74810) c5(74811, 74812, 74813, 74814, 74815, 74816) c6( ) c7(HQ, T, ac, Yc, s, dH, aE, e, r, aq, kp, ey, q, eA, cm, B); #c1 (DCLK1) c2(NP_004725) c3(serine/threonine-protein kinase DCLK1 isoform 1) c4(149089, 149090) c5(149091, 149092, 149093, 149094, 149095, 149096, 149097, 149098, 149099, 149100, 149101, 149102, 149103, 149104, 149105, 149106, 149107, 149108) c6( ) c7(HQ, T, ac, Yc, s, dH, aE, e, r, aq, kp, ey, q, eA, cm, B); #c1(DCLK1) c2(XP_006719956) c3(serine/threonine-protein kinase DCLK1 isoform X1) c4(293091, 293092, 293093) c5(293094, 293095, 293096, 293097, 293098, 293099) c6( ) c7(HQ, T, ac, Yc, s, dH, aE, e, r, aq, kp, ey, q, eA, cm, B); #c1(DCLK2) c2(NP_001035350) c3(serine/threonine-protein kinase DCLK2 isoform a) c4(32773) c5(32774, 32775, 32776, 32777, 32778, 32779) c6( ) c7(cd); #c1(DCLK2) c2(NP_001035351) c3(serine/threonine-protein kinase DCLK2 isoform b) c4(32780) c5(32781, 32782, 32783, 32784, 32785, 32786) c6( ) c7(cd); #c1(DCLK2) c2(XP_005262843) c3(serine/threonine-protein kinase DCLK2 isoform X2) c4(278145) c5(278146, 278147, 278148, 278149, 278150, 278151) c6( ) c7(cd); #c1(DCLK3) c2(NP_208382) c3(serine/threonine-protein kinase DCLK3) c4(228540) c5(228541, 228542, 228543, 228544, 228545, 228546) c6( ) c7(T, d, Vv, wR, azl, sa, Hr, Hp); #c1(DCLRE1A) c2(NP_055696) c3(DNA cross-link repair 1A protein) c4(182836) c5(182837, 182838, 182839, 182840, 182841, 182842, 182843, 182844, 182845, 182846) c6( ) c7(dA, os, ft, oW, oZ); #c1(DCLRE1C) c2(NP_001029027) c3(protein artemis isoform a) c4(30718, 30719, 30720) c5(30721, 30722, 30723, 30724, 30725, 30726, 30727, 30728, 30729, 30730, 30731, 30732, 30733, 30734, 30735) c6( ) c7(fM, dA, jh, aA, fd, V, q, o, eA, dP, bdJ, aT, h, pk, CB, aAi, oS, oV, Y, CM, Rr, hl, ff, bdH, r, CF, bdl, aF, en, QK, ck, ou, dB, cj, m, bt, xg); #c1(DCLRE1C) c2(XP_011517920) c3(protein artemis isoform X1) c4(311873, 311874) c5(311875, 311876, 311877, 311878, 311879, 311880, 311881, 311882, 311883, 311884, 311885, 311886, 311887, 311888, 311889, 311890, 311891, 311892) c6( ) c7(fM, dA, jh, aA, fd, V, q, o, eA, dP, bdJ, aT, h, pk, CB, aAi, oS, oV, Y, CM, Rr, hl, ff, bdH, r, CF, bdl, aF, en, QK, ck, ou, dB, cj, m, bt, xg); #c1(DCLRE1C) c2(XP_011517921) c3 (protein artemis isoform X2) c4(311893, 311894, 311895) c5(311896, 311897, 311898, 311899, 311900, 311901, 311902, 311903, 311904, 311905, 311906, 311907, 311908, 311909, 311910, 311911, 311912, 311913, 311914, 311915, 311916, 311917, 311918) c6( ) c7(fM, dA, jh, aA, fd, V, q, o, eA, dP, bdJ, aT, h, pk, CB, aAi, oS, oV, Y, CM, Rr, hl, ff, bdH, r, CF, bdl, aF, en, QK, ck, ou, dB, cj, m, bt, xg); #c1(DCLRE1C) c2(XP_011517922) c3(protein artemis isoform X3) c4( ) c5(311919, 311920, 311921, 311922, 311923, 311924) c6( ) c7(fM, dA, jh, aA, fd, V, q, o, eA, dP, bdJ, aT, h, pk, CB, aAi, oS, oV, Y, CM, Rr, hl, ff, bdH, r, CF, bdl, aF, en, QK, ck, ou, dB, cj, m, bt, xg); #c1(DCLRE1C) c2(XP_011517923) c3(protein artemis isoform X4) c4( ) c5(311925, 311926, 311927, 311928, 311929, 311930, 311931, 311932, 311933, 311934, 311935) c6( ) c7(fM, dA, jh, aA, fd, V, q, o, eA, dP, bdJ, aT, h, pk, CB, aAi, oS, oV, Y, CM, Rr, hl, ff, bdH, r, CF, bdl, aF, en, QK, ck, ou, dB, cj, m, bt, xg); #c1(DCN) c2(NP_598010) c3(decorin isoform a preproprotein) c4(235299, 235300, 235301, 235302, 235303, 235304) c5(235305, 235306, 235307, 235308, 235309, 235310, 235311, 235312, 235313, 235314, 235315, 235316, 235317, 235318, 235319, 235320, 235321, 235322, 235323, 235324, 235325, 235326, 235327, 235328, 235329, 235330, 235331, 235332, 235333, 235334, 235335, 235336, 235337, 235338, 235339, 235340, 235341, 235342, 235343, 235344) c6( ) c7(ID, bM, T, asg, ea, al, fd, bB, ci, aE, Bc, dv, ta, K, apj, H, eL, Qe, aW, pl, eF, aAW, eM, nB, tV, cj, Hb, m, R, fy, rt, dD, vl, zt, aw, aj, bdl, cd, bdK, amJ, dl, ey, pj, Dg, A, anP, aq, fq, bP, hL, ajd, hj, mg, rx, bm, B, AN, bL, fG, oe, cz, lj, v, j, uW, V, Eh, q, J, eA, lg, aT, als, zp, bq, ae, af, aCr, wr, bf, Pg, r, en, cl, t, pb, hc, agf, dy, EY, vi, kh, eq, ad, BY, dP, sN, le, uD, sj, d, ff, xs, iR, dr, LB, D, gk, RV, bE, aS); #c1(DCN) c2(NP_598011) c3(decorin isoform b precursor) c4(235345) c5(235346, 235347, 235348, 235349, 235350, 235351, 235352, 235353, 235354, 235355) c6( ) c7(ID, bM, T, asg, ea, al, fd, bB, ci, aE, Bc, dv, ta, K, apj, H, eL, Qe, aW, pl, eF, aAW, eM, nB, tV, cj, Hb, m, R, fy, rt, dD, vl, zt, aw, aj, bdL, cd, bdK, amJ, dL, ey, pj, Dg, A, anP, aq, fq, bP, hL, ajd, hj, mg, rx, bm, B, AN, bL, fG, De, cz, lj, v, j, uW, V, Eh, q, J, eA, lg, aT, als, zp, bq, ae, af, aCr, wr, bf, Pg, r, en, cl, t, pb, hc, agf, dy, EY, vi, kh, eq, ad, BY, dP, sN, le, uD, sj, d, ff, xs, iR, dr, LB, D, gk, RV, bE, aS); #c1(DCN) c2(NP_598012) c3(decorin isoform c precursor) c4(235356) c5(235357, 235358, 235359, 235360, 235361, 235362, 235363, 235364, 235365, 235366) c6( ) c7(ID, bM, T, asg, ea, al, fd, bB, ci, aE, Bc, dv, ta, K, apj, H, eL, Qe, aW, pl, eF, aAW, eM, nB, tV, cj, Hb, m, R, fy, rt, dD, vl, zt, aw, aj, bdl, cd, bdk, amJ, dl, ey, pj, Dg, A, anP, aq, fq, bP, hL, ajd, hj, mg, rx, bm, B, AN, bL, fG, oe, cz, lj, v, j, uW, V, Eh, q, J, eA, lg, aT, aHs, zp, bq, ae, af, aCr, wr, bf, Pg, r, en, cl, t, pb, hc, agf, dy, EY, vi, kh, eq, ad, BY, dP, sN, le, uD, sj, d, ff, xs, iR, dr, LB, D, gk, RV, bE, aS); #c1(DCN) c2(NP_598013) c3(decorin isoform d precursor) c4(235367) c5(235368, 235369, 235370, 235371, 235372, 235373, 235374, 235375, 235376, 235377) c6( ) c7(ID, bM, T, asg, ea, al, fd, bB, ci, aE, Bc, dv, ta, K, apj, H, eL, Qe, aW, pl, eF, aAW, eM, nB, tV, cj, Hb, m, R, fy, rt, dD, vl, zt, aw, aj, bdl, cd, bdk, amJ, dl, ey, pj, Dg, A, anP, aq, fq, bP, hL, ajd, hj, mg, rx, bm, B, AN, bL, fG, oe, cz, lj, v, j, uW, V, Eh, q, J, eA, lg, aT, aHs, zp, bq, ae, af, aCr, wr, bf, Pg, r, en, cl, t, pb, hc, agf, dy, EY, vi, kh, eq, ad, BY, dP, sN, le, uD, sj, d, ff, xs, iR, dr, LB, D, gk, RV, bE, aS); #c1(DCN) c2(NP_598014) c3(decorin isoform e precursor) c4( ) c5(235378, 235379, 235380, 235381, 235382, 235383) c6( ) c7(ID, bM, T, asg, ea, al, fd, bB, ci, aE, Bc, dv, ta, K, apj, H, eL, Qe, aW, pl, eF, aAW, eM, nB, tV, cj, Hb, m, R, fy, rt, dD, vl, zt, aw, aj, bdL, cd, bdk, amJ, dL, ey, pj, Dg, A, anP, aq, fq, bP, hL, ajd, hj, mg, rx, bm, B, AN, bL, fG, oe, cz, lj, v, j, uW, V, Eh, q, J, eA, lg, aT, aHs, zp, bq, ae, af, aCr, wr, bf, Pg, r, en, cl, t, pb, hc, agf, dy, EY, vi, kh, eq, ad, BY, dP, sN, le, uD, sj, d, ff, xs, iR, dr, LB, D, gk, RV, bE, aS); #c1(DCP1A) c2(NP_001277136) c3(mRNA-decapping enzyme IA isoform e) c4( ) c5(109113) c6( ) c7(yT, oe, K, c); #c1(DCP1A) c2(NP_060873) c3(mRNA-decapping enzyme (A isoform a) c4(198215) c5(198216, 198217, 198218, 198219, 198220, 198221, 198222) c6( ) c7(yT, oe, K, c); #c1(DCP1B) c2(NP_689853) c3(mRNA-decapping enzyme 1B) c4(244156) c5(244157, 244158, 244159, 244160, 244161, 244162, 244163) c6( ) c7(aC, W, bS, aS, ct, Z); #c1(DCP1B) c2(XP_011519229) c3(mRNA-decapping enzyme 1B isoform X1) c4(313822) c5(313823, 313824, 313825, 313826, 313827) c6( ) c7(aC, W, bS, aS, ct, Z); #c1(DCPS) c2(NP_054745) c3(m7GpppX diphosphatase) c4(177552) c5(177553, 177554, 177555, 177556, 177557, 177558) c6( ) c7(h, bM); #c1(DCST1) c2(NP_001137159) c3(DC-STAMP domain-containing protein 1 isoform 2) c4(55060) c5(55061, 55062, 55063, 55064, 55065, 55066) c6( ) c7(aw); #c1(DCST1) c2(NP_689707) c3(DC-STAMP domain-containing protein 1 isoform 1) c4(243635, 243636) c5(243637, 243638, 243639, 243640, 243641, 243642, 243643, 243644, 243645, 243646, 243647, 243648, 243649, 243650, 243651, 243652) c6( ) c7(aw); #c1(DCST2) c2(NP_653223) c3(DC-STAMP domain-containing protein 2) c4(239171, 239/72) c5(239173, 239174, 239175, 239176, 239177, 239178, 239179, 239180, 239181, 239182, 239183, 239184) c6( ) c7(aw); #c1(DCST2) c2(XP_011507491) c3(DC-STAMP domain-containing protein 2 isoform X2) c4( ) c5(297392) c6( ) c7(aw); #c1(DCSTAMP) c2(XP_005251132) c3(dendritic cell-specific transmembrane protein isoform X1) c4(270832) c5(270833, 270834, 270835, 270836, 270837, 270838) c6( ) c7(dP, aw, hl, bdM, bi, xa); #c1(DCSTAMP) c2(XP_011515626) c3(dendritic cell-specific transmembrane protein isoform X2) c4(308455) c5(308456, 308457, 308458, 308459, 308460, 308461) c6( ) c7(dP, aw, hl, bdM, bi, xa); #c1(DCT) c2(NP_001123361) c3(L-dopachrome tautomerase isoform 2 precursor) c4(47032, 47033) c5(47034, 47035, 47036, 47037, 47038, 47039) c6( ) c7(rp, d, Dy, bP, HP, Y, rl, hD, asM, qj, K, BM, aYs, BA, pC); #c1(DCT) c2(NP_001913) c3(L-dopachrome tautomerase isoform 1 precursor) c4(122147) c5(122148, 122149, 122150, 122151, 122152, 122153) c6( ) c7(rp, d, Dy, bP, HP, Y, rl, hD, asM, qj, K, BM, aYs, eA, pC); #c1(DCT) c2(XP_011519352) c3(L-dopachrome tautomerase isoform X1) c4( ) c5(314011, 314012, 314013, 314014, 314015, 314016) c6( ) c7(rp, d, Dy, bP, HP, Y, rl, hD, asM, qj, K, BM, aYs, eA, pC); #c1(DCTD) c2(NP_001012750) c3(deoxycytidylate deaminase isoform a) c4(25260, 25261) c5(25262, 25263, 25264, 25265, 25266, 25267) c6( ) c7(aC, ac); #c1(DCTD) c2(XP_005262835) c3(deoxycytidylate deaminase isoform X2) c4(278135) c5(278136, 278137, 278138, 278139, 278140, 278141, 278142, 278143, 278144) c6( ) c7(aC, ac); #c1(DCTD) c2(XP_011529976) c3(deoxycytidylate deaminase isoform X1) c4(325751, 325752, 325753, 325754, 325755, 325756) c5(325757, 325758, 325759, 325760, 325761, 325762, 325763, 325764, 325765, 325766, 325767, 325768, 325769, 325770, 325771, 325772, 325773, 325774, 325775, 325776, 325777, 325778, 325779, 325780, 325781, 325782, 325783, 325784, 325785, 325786, 325787, 325788, 325789, 325790, 325791, 325792, 325793, 325794, 325795, 325796, 325797, 325798, 325799) c6( ) c7(aC, ac); #c1(DCTN1) c2(NP_001128512) c3(dynactin subunit 1 isoform 3) c4(50128, 50129) c5(50130, 50131, 50132, 50133, 50134, 50135, 50136, 50137) c6( ) c7(bU, c, cA, Kx, avR, WY, Ol, aem, an, HC, aT, U, Kj, bdN, PJ, alS, V, Tq, aR, bdO); #c1(DCTN1) c2(NP_001128513) c3(dynactin subunit 1 isoform 4) c4(50138) c5(50139, 50140, 50141, 50142, 50143, 50144) c6( ) c7(bU, c, cA, Kx, avR, WY, Ol, aem, an, HC, aT, U, Kj, bdN, PJ, alS, V, Tq, aR, bdO); #c1(DCTN1) c2(NP_001177765) c3(dynactin subunit 1 isoform 5) c4(72980) c5(72981, 72982, 72983, 72984, 72985, 72986, 72987, 72988, 72989, 72990) c6( ) c7(bU, c, cA, Kx, avR, WY, Ol, aem, an, HC, aT, U, Kj, bdN, PJ, alS, V, Tq, aR, bdO); #c1(DCTN1) c2(NP_001177766) c3(dynactin subunit 1 isoform 6) c4(72991) c5(72992, 72993, 72994, 72995, 72996, 72997) c6( ) c7(bU, c, cA, Kx, avR, WY, Ol, aem, an, HC, aT, U, Kj, bdN, PJ, alS, V, Tq, aR, bdO); #c1(DCTN1) c2(NP_004073) c3(dynactin subunit 1 isoform 1) c4(143326, 143327, 143328, 143329) c5(143330, 143331, 143332, 143333, 143334, 143335, 143336, 143337, 143338, 143339, 143340, 143341, 143342, 143343, 143344, 143345, 143346, 143347, 143348, 143349, 143350, 143351, 143352, 143353, 143354, 143355, 143356, 143357, 143358, 143359) c6( ) c7(bU, c, cA, Kx, avR, WY, Ol, aem, an, HC, aT, U, Kj, bdN, PJ, alS, V, Tq, aR, bdO); #c1(DCTN1) c2(NP_075408) c3(dynactin subunit 1 isoform 2) c4(213858) c5( ) c6( ) c7(bU, c, CA, Kx, avR, WY, Ol, aem, an, HC, aT, U, Kj, bdN, PJ, alS, V, Tq, aR, bdO); #c1(DCTN2) c2(NP_001248341) c3(dynactin subunit 2 isoform 2) c4(93723) c5( ) c6( ) c7(eF, T, eH); #c1(DCTN2) c2(NP_001248342) c3(dynactin subunit 2 isoform 3) c4(93724) c5(93725, 93726, 93727, 93728, 93729, 93730) c6( ) c7(eF, T, eH); #c1(DCTN2) c2(NP_006391) c3(dynactin subunit 2 isoform 1) c4(164628, 164629) c5(164630, 164631, 164632, 164633, 164634, 164635, 164636, 164637, 164638, 164639, 164640, 164641, 164642, 164643, 164644, 164645, 164646, 164647, 164648) c6( ) c7(eF, T, eH); #c1(DCTN2) c2(XP_011536063) c3(dynactin subunit 2 isoform X1) c4(333383) c5(333384, 333385, 333386, 333387, 333388) c6( ) c7(eF, T, eH); #c1(DCTN3) c2(NP_001268354) c3(dynactin subunit 3 isoform 3) c4(99717, 99718) c5(99719, 99720, 99721, 99722, 99723, 99724, 99725, 99726, 99727, 99728, 99729, 99730) c6( ) c7(T, aNB, DK, nv, bdP, Pg, B, bdQ); #c1(DCTN3) c2(NP_001268355) c3(dynactin subunit 3 isoform 4) c4( ) c5(99731, 99732, 99733, 99734, 99735, 99736, 99737, 99738, 99739, 99740, 99741, 99742, 99743, 99744, 99745, 99746, 99747) c6( ) c7(T, aNB, DK, nv, bdP, Pg, B, bdQ); #c1(DCTN3) c2(NP_001268356) c3(dynactin subunit 3 isoform 5) c4( ) c5(99748) c6( ) c7(T, aNB, DK, nv, bdP, Pg, B, bdQ); #c1(DCTN3) c2(NP_009165) c3(dynactin subunit 3 isoform 1) c4(171218, 171219, 171220, 17/221) c5(171222, 171223, 171224, 171225, 171226, 171227) c6( ) c7(T, aNB, DK, nv, bdP, Pg, B, bdQ); #c1(DCTN3) c2(NP_077324) c3(dynactin subunit 3 isoform 2) c4(215241) c5(215242, 215243, 215244, 215245, 215246, 215247) c6( ) c7(T, aNB, DK, nv, bdP, Pg, B, bdQ); #c1(DCTN4) c2(NP_001129115) c3(dynactin subunit 4 isoform a) c4(50806) c5(50807, 50808, 50809, 50810, 50811, 50812) c6( ) c7(kV, Kd, T, bM, pF, if, GC, To, v, BF, ci, q, ad, Kx, jz, zw, WY, aT, h, K, Wr, ae, af, d, aW, ff, az, alR, r, aq, aaE, kp, en, g, bb, kP, c, eM, U, Kj, bdR, bi, xg, B, ao); #c1(DCTN4) c2(NP_001129116) c3(dynactin subunit 4 isoform c) c4(50813, 50814, 50815) c5(50816, 50817, 50818, 50819, 50820, 50821, 50822, 50823, 50824, 50825, 50826, 50827, 50828, 50829, 50830) c6( ) c7(kV, Kd, T, bM, pF, if, GC, To, v, BF, ci, q, ad, Kx, jz, zw, WY, aT, h, K, Wr, ae, af, d, aW, ff, az, alR, r, aq, aaE, kp, en, g, bb, kP, c, eM, U, Kj, bdR, bi, xg, B, ao); #c1(DCTN4) c2(NP_057305) c3(dynactin subunit 4 isoform b) c4(190017) c5(190018, 190019, 190020, 190021, 190022, 190023, 190024, 190025, 190026) c6( ) c7(kV, Kd, T, bM, pF, if, GC, To, v, BF, ci, q, ad, Kx, jz, zw, WY, aT, h, K, Wr, ae, af, d, aW, ff, az, alR, r, aq, aaE, kp, en, g, bb, kP, c, eM, U, Kj, bdR, bi, xg, B, ao); #c1(DCTN5) c2(NP_001185940) c3(dynactin subunit 5 isoform 2) c4(76798) c5(76799, 76800, 76801, 76802, 76803, 76804) c6( ) c7(Hx, ac, c, U, aT, cz, h, aj, j, bK, fr, y, gD, aR); #c1(DCTN5) c2(NP_115875) c3(dynactin subunit 5 isoform 1) c4(224425) c5(224426, 224427, 224428, 224429, 224430, 224431) c6( ) c7(Hx, ac, c, U, aT, cz, h, aj, j, bK, fr, y, gD, aR); #c1(DCTNG) c2(NP_006562) c3(dynactin subunit 6) c4(166243) c5(166244, 166245, 166246, 166247, 166248, 166249) c6( ) c7(T, ea, if, fd, bB, ci, aE, il, Qk, K, eL, bc, xQ, yo, aW, as, cm, bw, GR, eM, nB, dc, go, vA, cj, m, dD, NT, n, fr, qN, jz, h, dL, Oz, ge, aq, zZ, iO, bP, hR, it, B, fM, aDE, aA, q, eA, XX, aT, dz, ae, af, jf, s, iU, Y, r, en, cl, t, ik, aEp, ck, nM, iM, dy, bt, On, hS, hl, Ke, pY, jh, eq, ad, hX, hd, ib, fb, d, je, et, ff, apb, af, aTa, bz, bE, aS, EB); #c1(DCUN1D1) c2(NP_065691) c3(DCN1-like protein 1) c4(204971, 204972) c5(204973, 204974, 204975, 204976, 204977, 204978, 204979, 204980, 204981, 204982, 204983, 204984, 204985) c6( ) c7(T, d, eM, iE, WY, ff, iv, aaE, ci, en, g, it, eA); #c1(DCUN1D1) c2(XP_011511215) c3(DCN1-like protein 1 isoform X1) c4(301696) c5( ) c6( ) c7(T, d, eM, iE, WY, ff, iv, aaE, ci, en, g, it, eA); #c1(DCX) c2(NP_000546) c3(neuronal migration protein doublecortin isoform a) c4(12623, 12624, 12625) c5(12626, 12627, 12628, 12629, 12630, 12631, 12632, 12633, 12634, 12635, 12636, 12637, 12638, 12639, 12640) c6( ) c7(T, hl, Tb, qx, ha, jx, qy, n, bdV, nA, Fc, afz, eA, ad, iC, Hx, hb, K, bdS, Lg, jf, d, aeR, Y, Ls, FD, qL, Xj, as, cm, Hp, bdW, bdT, WO, afZ, dB, wl, FB, bdU); #c1(DCX) c2(NP_835365) c3(neuronal migration protein doublecortin isoform b) c4(253485) c5(253486, 253487, 253488, 253489, 253490, 253491, 253492, 253493, 253494, 253495) c6( ) c7(T, hl, Tb, qx, ha, jx, qy, n, bdV, nA, Fc, afz, eA, ad, iC, Hx, hb, K, bdS, Lg, jf, d, aeR, Y, Ls, FD, qL, Xj, as, cm, Hp, bdW, bdT, WO, afZ, dB, wO, FB, bdU); #c1(DCX) c2(NP_835366) c3(neuronal migration protein doublecortin isoform c) c4(253496) c5(253497, 253498, 253499, 253500, 253501, 253502, 253503, 253504) c6( ) c7(T, hl, Tb, qx, ha, jx, qy, n, bdV, nA, Fc, afz, eA, ad, iC, Hx, hb, K, bdS, Lg, jf, d, aeR, Y, Ls, FD, qL, Xj, as, cm, Hp, bdW, bdT, WO, afZ, dB, wl, FB, bdU); #c1(DCX) c2(XP_011529182) c3(neuronal migration protein doublecortin isoform X3) c4( ) c5(324586, 324587, 324588, 324589, 324590) c6( ) c7(T, hl, Tb, qx, ha, jx, qy, n, bdV, nA, Fc, afz, eA, ad, iC, Hx, hb, K, bdS, Lg, jf, d, aeR, Y, Ls, FD, qL, Xj, as, cm, Hp, bdW, bdT, WO, afZ, dB, wD, FB, bdU); #c1(DCXR) c2(NP_057370) c3(L-xylulose reductase isoform 1) c4(190419) c5(190420, 190421, 190422, 190423, 190424, 190425) c6( ) c7(ai, bdX, ac, xE, lH, v, Fb, nA, f, ae, B, ad, ek); #c1(DDAH1) c2(XP_011539460) c3 (N(G),N(G)-dimethylarginine dimethylaminohydrolase 1 isoform X3) c4(337642) c5(337643, 337644, 337645, 337646, 337647, 337648, 337649, 337650, 337651, 337652, 337653, 337654, 337655) c6( ) c7(bL, fU, ac, wE, fG, e, aw, hl, cz, sM, cR, eb, QL, j, dy, cm, lC, uC, tw); #c1(DDAH2) c2(NP_001289937) c3(N(G),N(G)-dimethylarginine dimethylaminohydrolase 2) c4(114715, 114716, 114717, 114718, 114719) c5(114720, 114721, 114722, 114723, 114724, 114725, 114726, 114727, 114728, 114729, 114730, 114731, 114732, 114733, 114734, 114735, 114736, 114737, 114738, 114739, 114740, 114741, 114742, 114743, 114744, 114745, 114746, 114747, 114748, 114749, 114750, 114751, 114752, 114753, 114754, 114755, 114756, 114757, 114758, 114759, 114760, 114761, 114762, 114763, 114764, 114765, 114766, 114767, 114768, 114769, 114770, 114771, 114772, 114773, 114774, 114775, 114776, 114777, 114778, 114779, 114780, 114781, 114782, 114783, 114784, 114785, 114786, 114787, 114788, 114789, 114790, 114791, 114792, 114793, 114794, 114795, 114796, 114797, 114798, 114799, 114800, 114801, 114802, 114803, 114804, 114805, 114806, 114807, 114808, 114809, 114810, 114811, 114812, 114813, 114814, 114815, 114816, 114817, 114818, 114819, 114820, 114821, 114822, 114823, 114824, 114825, 114826, 114827, 114828, 114829, 114830, 114831, 1/4832, 114833, 1/4834, 114835, 114836, 114837, 114838, 114839, 114840, 114841, 114842, 1/4843, 114844, 114845, 114846, 114847, 114848, 114849, 114850, 114851, 114852, 114853, 114854, 114855, 114856, 114857, 114858, 114859, 114860, 114861, 114862, 114863, 114864, 114865, 114866, 114867, 114868) c6( ) c7(bL, dD, ea, fG, e, aw, cz, sM, cR, aj, dG, j, v, eq, aY, FU, K, A, Y, eZ, uC, ac, wE, fP, dy); #c1(DDB1) c2(NP_001914) c3(DNA damage-binding protein 1) c4(122154, 122155, 122156, 122157) c5(122158, 122159, 122160, 122161, 122162, 122163, 122164, 122165, 122166, 122167, 122168, 122169, 122170, 122171, 122172, 122173, 122174, 122175, 122176, 122177, 122178, 122179, 122180, 122181) c6( ) c7(jx, CT, aA, bB, K, m, aVV, t, B, hX); #c1(DDB2) c2(NP_000098) c3(DNA damage-binding protein 2) c4(1736, 1737, 1738) c5(1739, 1740, 1741, 1742, 1743, 1744, 1745, 1746, 1747, 1748, 1749, 1750, 1751, 1752, 1753, 1754, 1755, 1756) c6( ) c7(T, d, BN, bE, aW, Y, yk, hX, CT, aT, bB, hy, dL, K, m, aVV, t, V, bz, ne, bdY); #c1(DDB2) c2(NP_001287663) c3(DNA damage-binding protein 2 isoform DI) c4(112485) c5(112486, 112487, 112488, 112489, 112490, 112491) c6( ) c7(T, d, BN, bE, aW, Y, yk, hX, CT, aT, bB, hy, dL, K, m, aVV, t, V, bz, ne, bdY); #c1(DDC) c2(NP_000781) c3(aromatic-L-amino-acid decarboxylase isoform 1) c4(15686) c5(15687, 15688, 15689, 15690, 15691, 15692, 15693, 15694, 15695, 15696, 15697, 15698, 15699, 15700, 15701) c6( ) c7(T, eR, bdZ, uG, aA, mS, n, bS, ti, ur, uu, ah, qi, q, ad, ft, bea, aji, hb, mW, HC, dV, bK, an, mY, mV, he, ae, na, mU, B, Gv, ff, LO, fQ, hN, aF, en, g, t, vo, cm, mZ, iK, bN, mX, cv, hq, Nb, m, mT); #c1(DDC) c2(NP_001229817) c3(aromatic-L-amino-acid decarboxylase isoform 4) c4(85321) c5(85322, 85323, 85324, 85325, 85326, 85327) c6( ) c7(T, eR, bdZ, uG, aA, mS, n, bS, ti, ur, uu, ah, qi, q, ad, ft, bea, aji, hb, mW, HC, dV, bK, an, mY, mV, he, ae, na, mU, B, Gv, ff, LO, fl, hN, aF, en, g, t, vo, cm, mZ, iK, bN, mX, cv, hq, Nb, m, mT); #c1(DDC) c2(NP_001229818) c3(aromatic-L-amino-acid decarboxylase isoform 5) c4(85328) c5(85329, 85330, 85331, 85332, 85333, 85334) c6( ) c7(T, eR, bdZ, uG, aA, mS, n, bS, ti, ur, uu, ah, qi, q, ad, ft, bea, aji, hb, mW, HC, dV, bK, an, mY, mV, he, ae, na, mU, B, Gv, ff, LO, fQ, hN, aF, en, g, t, vo, cm, mZ, ik, bN, mX, cv, hq, Nb, m, mT); #c1(DDC) c2(NP_001229819) c3(aromatic-L-amino-acid decarboxylase isoform 6) c4(85335, 85336) c5(85337, 85338, 85339, 85340, 85341, 85342) c6( ) c7(T, eR, bdZ, uG, aA, mS, n, bS, ti, ur, uu, ah, qi, q, ad, ft, bea, aji, hb, mW, HC, dV, bK, an, mY, mV, he, ae, na, mU, B, Gv, ff, LO, fQ, hN, aF, en, g, t, vo, cm, mZ, ik, bN, mX, cv, hq, Nb, m, mT); #c1(DDC) c2(XP_005271802) c3(aromatic-L-amino-acid decarboxylase isoform X1) c4(283597) c5(283598, 283599, 283600, 283601, 283602, 283603) c6( ) c7(T, eR, bdZ, uG, aA, mS, n, bS, ti, ur, uu, ah, qi, q, ad, ft, bea, aji, hb, mW, HC, dV, bk, an, mY, mV, he, ae, na, mU, B, Gv, ff, LO, fQ, hN, aF, en, g, t, vo, cm, mZ, ik, bN, mX, cv, hq, Nb, m, mT); #c1(DDHD1) c2(NP_001153619) c3(phospholipase DDHD1 isoform b) c4(60764) c5(60765, 60766, 60767, 60768, 60769, 60770) c6( ) c7(bcR, xV, oe, bcS, beb); #c1(DDHD1) c2(NP_001153620) c3(phospholipase DDHD1 isoform c) c4(60771, 60772) c5(60773, 60774, 60775, 60776, 60777, 60778, 60779, 60780, 60781, 60782, 60783, 60784) c6( ) c7(bcR, xV, oe, bcS, beb); #c1(DDHD1) c2(NP_085140) c3(phospholipase DDHD1 isoform a) c4(219402) c5(219403, 219404, 219405, 219406, 219407, 219408) c6( ) c7(bcR, xV, oe, bcS, beb); #c1(DDHD1) c2(XP_005268160) c3(phospholipase DDHD1 isoform X4) c4(281630) c5( ) c6( ) c7(bcR, xV, oe, bcS, beb); #c1(DDHD2) c2(NP_001157706) c3(phospholipase DDHD2 isoform 2) c4( ) c5(62416, 62417, 62418, 62419, 62420, 62421, 62422, 62423, 62424, 62425, 62426, 62427) c6( ) c7(bec, aV, bcS); #c1(DDHD2) c2(XP_005273511) c3(phospholipase DDHD2 isoform X1) c4(284639, 284640) c5(284641, 284642, 284643, 284644, 284645, 284646, 284647, 284648, 284649, 284650, 284651, 284652, 284653, 284654, 284655, 284656) c6( ) c7(bec, aV, bcS); #c1(DDHD2) c2(XP_005273513) c3(phospholipase DDHD2 isoform X2) c4(284657, 284658, 284659) c5( ) c6( ) c7(bec, aV, bcS); #c1(DDHD2) c2(XP_005273514) c3 (phospholipase DDHD2 isoform X3) c4( ) c5(284660, 284661, 284662, 284663, 284664, 284665) c6( ) c7(bec, aV, bcS); #c1(DDI1) c2(NP_001001711) c3(protein DDI1 homolog 1) c4(18966) c5(18967, 18968, 18969, 18970, 18971, 18972) c6( ) c7(aw); #c1(DDIAS) c2(NP_659455) c3(DNA damage-induced apoptosis suppressor protein) c4(240011, 240012, 240013, 240014, 240015, 240016) c5(240017, 240018, 240019, 240020, 240021, 240022, 240023, 240024, 240025, 240026, 240027, 240028, 240029, 240030, 240031, 240032, 240033, 240034, 240035, 240036, 240037, 240038, 240039, 240040, 240041, 240042, 240043, 240044, 240045, 240046, 240047, 240048, 240049, 240050, 240051, 240052, 240053, 240054, 240055, 240056) c6( ) c7(aW, aT); #c1(DDIT3) c2(NP_001181983) c3(DNA damage-inducible transcript 3 protein isoform 1) c4(74466, 74467) c5(74468, 74469, 74470, 74471, 74472, 74473, 74474, 74475, 74476, 74477, 74478, 74479, 74480, 74481, 74482, 74483, 74484) c6( ) c7(T, aC, YU, hV, if, bO, fd, bB, ci, aon, K, pk, aa, eG, aW, wf, hl, cm, bw, QA, eM, nB, iT, vA, U, cj, uJ, nw, m, aif, n, aj, qN, aDJ, ju, jy, Xh, dx, A, TH, bed, Ni, aq, aHw, iN, op, bP, mg, hj, at, E, va, bee, cB, B, bL, fM, Dh, jx, aA, fF, hy, v, j, bX, q, J, eA, aUy, aT, cy, rz, ae, eu, iU, oV, Y, axW, r, en, t, iK, aXi, ck, c, au, bt, ao, gV, Ve, id, iW, ha, jh, ad, afb, hX, hd, Rq, Ps, xX, d, hG, ff, aF, iR, adj, bz, LB, bE, do, uR, QC, ei, X); #c1(DDIT3) c2(NP_001181986) c3(DNA damage-inducible transcript 3 protein isoform 2) c4( ) c5(74485, 74486, 74487, 74488, 74489, 74490, 74491, 74492) c6( ) c7(T, aC, YU, hV, if, bO, fd, bB, ci, aon, K, pk, aa, eG, aW, wf, hl, cm, bw, QA, eM, nB, iT, vA, U, cj, uJ, nw, m, aiF, n, aj, qN, aOJ, ju, jy, Xh, dx, A, TH, bed, Ni, aq, aHw, iN, op, bP, mg, hj, at, E, va, bee, cB, B, bL, fM, Dh, jx, aA, fF, hy, v, j, bX, q, J, eA, aHy, aT, cy, rz, ae, eu, iU, oV, Y, axW, r, en, t, ik, aXi, ck, c, au, bt, ao, gV, Ve, id, iW, ha, jh, ad, afb, hX, hd, Rq, Ps, xX, d, hG, ff, aF, iR, adj, bz, LB, bE, dO, uR, QC, ei, X); #c1(DDIT3) c2(XP_011536308) c3(DNA damage-inducible transcript 3 protein isoform X1) c4(333662) c5(333663, 333664) c6( ) c7(T, aC, YU, hV, if, bO, fd, bB, ci, aon, K, pk, aa, eG, aW, wf , hl, cm, bw, QA, eM, nB, iT, vA, U, cj, uJ, nw, m, aiF, n, aj, qN, aDJ, ju, jy, Xh, dx, A, TH, bed, Ni, aq, aUw, iN, op, bP, mg, hj, at, E, va, bee, cB, B, bL, fM, Dh, jx, aA, fF, hy, v, j, bX, q, J, eA, aUy, aT, cy, rz, ae, eu, iU, oV, Y, axW, r, en, t, ik, aXi, ck, c, au, bt, ao, gV, Ve, id, iW, ha, jh, ad, afb, hX, hd, Rq, Ps, xX, d, hG, ff, aF, iR, adj, bz, LB, bE, dO, uR, QC, ei, X); #c1(DDIT4) c2(NP_061931) c3(DNA damage-inducible transcript 4 protein) c4(201202) c5(201203, 201204, 201205, 201206, 201207, 201208) c6( ) c7(ck, T, hl, W, af, iT, ff, ik, HC, bO, fd, K, en, an, bt, ae, avn, ad, hq); #c1(DDIT4L) c2(NP_660287) c3(DNA damage-inducible transcript 4-like protein) c4(240643, 240644, 240645) c5(240646, 240647, 240648, 240649, 240650, 240651, 240652, 240653, 240654, 240655, 240656, 240657, 240658, 240659, 240660, 240661, 240662, 240663) c6( ) c7(Y, bX); #c1(DDN) c2(NP_055901) c3(dendrin) c4(184333) c5(184334, 184335, 184336, 184337, 184338, 184339) c6( ) c7(ah, dy); #c1(DDN) c2(XP_011536357) c3(dendrin isoform X1) c4( ) c5(333680, 333681, 333682, 333683, 333684) c6( ) c7(ah, dy); #c1(DDO) c2(NP_003640) c3(D-aspartate oxidase isoform a) c4(139816, 139817) c5(139818, 139819, 139820, 139821, 139822, 139823, 139824, 139825, 139826, 139827, 139828, 139829) c6( ) c7(fq); #c1(DDO) c2(NP_004023) c3(D-aspartate oxidase isoform b) c4(142922) c5(142923, 142924, 142925, 142926, 142927, 142928) c6( ) c7(fq); #c1(DDR1) c2(NP_001189450) c3(epithelial discoidin domain-containing receptor 1 isoform 4 precursor) c4( ) c5(79411, 79412, 79413, 79414, 79415, 79416, 79417, 79418, 79419, 79420, 79421, 79422, 79423, 79424, 79425, 79426, 79427, 79428, 79429, 79430, 79431, 79432, 79433, 79434, 79435, 79436, 79437, 79438, 79439, 79440, 79441, 79442, 79443, 79444, 79445, 79446, 79447, 79448, 79449, 79450, 79451, 79452, 79453, 79454, 79455, 79456, 79457, 79458, 79459, 79460, 79461, 79462, 79463, 79464, 79465, 79466, 79467, 79468, 79469, 79470, 79471, 79472, 79473, 79474, 79475, 79476, 79477, 79478, 79479, 79480, 79481, 79482, 79483, 79484, 79485, 79486, 79487, 79488, 79489, 79490, 79491, 79492, 79493, 79494, 79495, 79496, 79497, 79498, 79499, 79500, 79501, 79502, 79503, 79504, 79505, 79506, 79507, 79508, 79509, 79510, 79511, 79512, 79513, 79514, 79515, 79516, 79517, 79518, 79519, 79520, 79521, 79522, 79523, 79524, 79525, 79526, 79527, 79528, 79529, 79530, 79531, 79532, 79533, 79534, 79535, 79536, 79537, 79538, 79539, 79540, 79541, 79542, 79543, 79544, 79545, 79546, 79547, 79548, 79549, 79550, 79551, 79552, 79553, 79554, 79555, 79556, 79557, 79558, 79559, 79560, 79561, 79562, 79563, 79564, 79565, 79566, 79567, 79568, 79569, 79570, 79571, 79572, 79573, 79574, 79575, 79576, 79577, 79578, 79579, 79580, 79581, 79582, 79583, 79584, 79585, 79586, 79587, 79588, 79589, 79590, 79591, 79592, 79593, 79594, 79595, 79596, 79597, 79598, 79599, 79600, 79601, 79602, 79603, 79604, 79605, 79606, 79607, 79608, 79609, 79610, 79611, 79612, 79613, 79614, 79615, 79616, 79617, 79618, 79619, 79620, 79621, 79622, 79623, 79624, 79625, 79626, 79627, 79628, 79629, 79630, 79631, 79632, 79633, 79634, 79635, 79636, 79637, 79638, 79639, 79640, 79641, 79642, 79643, 79644, 79645, 79646, 79647, 79648, 79649, 79650, 79651, 79652, 79653, 79654, 79655, 79656, 79657, 79658, 79659, 79660, 79661, 79662, 79663, 79664, 79665, 79666, 79667, 79668, 79669, 79670, 79671, 79672, 79673, 79674, 79675, 79676, 79677, 79678, 79679, 79680, 79681, 79682, 79683, 79684, 79685, 79686, 79687, 79688, 79689, 79690, 79691, 79692, 79693, 79694, 79695, 79696, 79697, 79698, 79699, 79700, 79701, 79702, 79703, 79704, 79705, 79706, 79707, 79708, 79709, 79710, 79711, 79712, 79713, 79714, 79715, 79716, 79717, 79718, 79719, 79720, 79721, 79722, 79723, 79724, 79725, 79726, 79727, 79728, 79729, 79730, 79731, 79732, 79733, 79734, 79735, 79736, 79737, 79738, 79739, 79740, 79741, 79742, 79743, 79744, 79745, 79746, 79747, 79748, 79749, 79750, 79751, 79752, 79753, 79754, 79755, 79756, 79757, 79758, 79759, 79760, 79761, 79762, 79763) c6(79764, 79765, 79766, 79767, 79768, 79769, 79770, 79771, 79772, 79773, 79774, 79775, 79776, 79777, 79778, 79779, 79780, 79781, 79782, 79783, 79784, 79785, 79786, 79787, 79788, 79789, 79790, 79791, 79792, 79793, 79794, 79795, 79796, 79797, 79798, 79799, 79800, 79801, 79802, 79803, 79804, 79805, 79806, 79807, 79808, 79809, 79810, 79811, 79812, 79813, 79814, 79815, 79816, 79817, 79818, 79819, 79820, 79821, 79822, 79823, 79824, 79825, 79826, 79827, 79828, 79829, 79830, 79831, 79832, 79833, 79834, 79835, 79836, 79837, 79838, 79839, 79840, 79841, 79842, 79843, 79844, 79845, 79846, 79847, 79848, 79849) c7(T, dD, ea, al, jx, l, eX, cl, v, qj, ci, vQ, eq, aoj, zP, Ul, pC, q, o, cd, afo, ad, eA, av, jz, eW, aT, eV, hn, vB, K, ey, H, hH, ae, af, bc, aRY, aa, yo, A, ND, aW, Y, cr, r, eZ, aq, lo, si, g, pt, t, bz, LB, ck, gk, es, tj, eM, iA, dB, hc, mx, hL, HR, k, Hb, R, m, bt, B, L); #c1(DDR1) c2(NP_001189451) c3(epithelial discoidin domain-containing receptor 1 isoform 5 precursor) c4(79850) c5(79851, 79852, 79853, 79854, 79855, 79856, 79857, 79858, 79859, 79860, 79861, 79862, 79863, 79864, 79865, 79866, 79867, 79868, 79869, 79870, 79871, 79872, 79873, 79874, 79875, 79876, 79877, 79878, 79879, 79880, 79881, 79882, 79883, 79884, 79885, 79886, 79887, 79888, 79889, 79890, 79891, 79892, 79893, 79894, 79895, 79896, 79897, 79898, 79899, 79900, 79901, 79902, 79903, 79904) c6(79905, 79906, 79907, 79908, 79909, 79910, 79911, 79912, 79913, 79914, 79915, 79916, 79917, 79918, 79919, 79920) c7(T, dD, ea, al, jx, l, eX, cl, v, qj, ci, vQ, eq, aoj, zP, UI, pC, q, o, cd, afo, ad, eA, av, jz, eW, aT, eV, hn, vB, K, ey, H, hH, ae, af, bc, aRY, aa, yo, A, ND, aW, Y, cr, r, eZ, aq, lo, si, g, pt, t, bz, LB, ck, gk, es, tj, eM, iA, dB, hc, mx, hL, hR, k, Hb, R, m, bt, B, L); #c1(DDR1) c2(NP_001189452) c3(epithelial discoidin domain-containing receptor 1 isoform 6 precursor) c4(79921) c5(79922, 79923, 79924, 79925, 79926, 79927, 79928, 79929, 79930, 79931, 79932, 79933, 79934, 79935, 79936, 79937, 79938, 79939, 79940, 79941, 79942, 79943, 79944, 79945, 79946, 79947, 79948, 79949, 79950, 79951, 79952, 79953, 79954, 79955, 79956, 79957, 79958, 79959, 79960, 79961, 79962, 79963, 79964, 79965, 79966, 79967, 79968, 79969, 79970, 79971, 79972, 79973, 79974, 79975, 79976, 79977, 79978, 79979, 79980, 79981, 79982, 79983, 79984, 79985, 79986, 79987, 79988, 79989, 79990, 79991, 79992, 79993, 79994, 79995, 79996, 79997, 79998, 79999, 80000, 80001, 80002, 80003, 80004, 80005, 80006, 80007, 80008, 80009, 80010, 80011, 80012, 80013, 80014, 80015, 80016, 80017, 80018, 80019, 80020, 80021, 80022, 80023, 80024, 80025, 80026, 80027, 80028, 80029, 80030, 80031, 80032, 80033, 80034, 80035, 80036, 80037, 80038, 80039, 80040, 80041, 80042, 80043, 80044, 80045, 80046, 80047, 80048, 80049, 80050, 80051, 80052, 80053, 80054, 80055, 80056, 80057, 80058, 80059, 80060, 80061, 80062, 80063, 80064, 80065, 80066, 80067, 80068, 80069, 80070, 80071, 80072, 80073, 80074, 80075, 80076, 80077, 80078, 80079, 80080, 80081, 80082, 80083, 80084, 80085, 80086, 80087, 80088, 80089, 80090, 80091, 80092, 80093, 80094, 80095, 80096, 80097, 80098, 80099, 80100, 80101, 80102, 80103, 80104, 80105, 80106, 80107, 80108, 80109, 80110, 80111, 80112, 80113, 80114, 80115, 80116, 80117, 80118, 80119, 80120, 80121, 80122, 80123, 80124, 80125, 80126, 80127, 80128, 80129, 80130, 80131, 80132, 80133, 80134) c6(80135, 80136, 80137, 80138, 80139, 80140, 80141, 80142, 80143, 80144, 80145, 80146, 80147, 80148, 80149, 80150, 80151, 80152, 80153, 80154, 80155, 80156, 80157, 80158, 80159, 80160, 80161, 80162, 80163, 80164, 80165, 80166, 80167, 80168, 80169, 80170, 80171, 80172, 80173, 80174, 80175, 80176, 80177, 80178, 80179, 80180, 80181, 80182, 80183, 80184, 80185, 80186, 80187, 80188) c7(T, dD, ea, al, jx, l, eX, cl, v, qj, ci, vQ, eq, aoj, zP, Ul, pC, q, o, cd, afo, ad, eA, av, jz, eW, aT, eV, hn, vB, K, ey, H, hH, ae, af, bc, aRY, aa, yo, A, ND, aW, Y, cr, r, eZ, aq, lo, si, g, pt, t, bz, LB, ck, gk, es, tj, eM, iA, dB, hc, mx, hL, hR, k, Hb, R, m, bt, B, L); #c1(DDR1) c2(NP_001945) c3(epithelial discoidin domain-containing receptor 1 isoform 1 precursor) c4(122394, 122395, 122396, 122397, 122398) c5(122399, 122400, 122401, 122402, 122403, 122404, 122405, 122406, 122407, 122408, 122409, 122410, 122411, 122412, 122413, 122414, 122415, 122416, 122417, 122418, 122419, 122420, 122421, 122422, 122423, 122424, 122425, 122426, 122427) c6(122428, 122429, 122430, 122431, 122432, 122433, 122434, 122435, 122436, 122437) c7(T, dD, ea, al, jx, l, eX, cl, v, qj, ci, vQ, eq, aoj, zP, Ul, pC, q, o, cd, afo, ad, eA, av, jz, eW, aT, eV, hn, vB, K, ey, H, hH, ae, af, bc, aRY, aa, yo, A, ND, aW, Y, cr, r, eZ, aq, lo, si, g, pt, t, bz, LB, ck, gk, es, tj, eM, iA, dB, hc, mx, hL, hR, k, Hb, R, m, bt, B, L); #c1(DDR1) c2(NP_054699) c3(epithelial discoidin domain-containing receptor 1 isoform 2 precursor) c4(177286, 177287, 177288, 177289, 177290, 177291, 177292) c5( ) c6( ) c7(T, dD, ea, al, jx, l, eX, cl, v, qj, ci, vQ, eq, aoj, zP, Ul, pC, q, o, cd, afo, ad, eA, av, jz, eW, aT, eV, hn, vB, K, ey, H, hH, ae, af, bc, aRY, aa, yo, A, ND, aW, Y, cr, r, eZ, aq, lo, si, g, pt, t, bz, LB, ck, gk, es, tj, eM, iA, dB, hc, mx, hL, hR, k, Hb, R, m, bt, B, L); #c1(DDR1) c2(XP_006715248) c3(epithelial discoidin domain-containing receptor 1 isoform X7) c4( ) c5(289011, 289012, 289013, 289014, 289015, 289016, 289017, 289018, 289019, 289020, 289021, 289022, 289023, 289024, 289025, 289026, 289027, 289028, 289029, 289030, 289031, 289032, 289033, 289034, 289035, 289036, 289037, 289038, 289039, 289040, 289041, 289042, 289043, 289044, 289045, 289046, 289047, 289048, 289049, 289050, 289051, 289052, 289053, 289054, 289055, 289056, 289057, 289058, 289059, 289060, 289061, 289062, 289063, 289064, 289065, 289066, 289067) c6(289068, 289069, 289070, 289071, 289072, 289073, 289074, 289075, 289076, 289077, 289078, 289079, 289080, 289081, 289082, 289083, 289084, 289085, 289086, 289087, 289088, 289089) c7(T, dD, ea, al, jx, l, eX, cl, v, qj, ci, vQ, eq, aoj, zP, Ul, pC, q, o, cd, afo, ad, eA, av, jz, eW, aT, eV, hn, vB, K, ey, H, hH, ae, af, bc, aRY, aa, yo, A, ND, aW, Y, cr, r, eZ, aq, lo, si, g, pt, t, bz, LB, ck, gk, es, tj, eM, iA, dB, hc, mx, hL, hR, k, Hb, R, m, bt, B, L); #c1(DDR1) c2(XP_011513184) c3(epithelial discoidin domain-containing receptor 1 isoform X6) c4(305115, 305116, 305117) c5( ) c6( ) c7(T, dD, ea, al, jx, l, eX, cl, v, qj, ci, vl, eq, aoj, zP, Ul, pC, q, o, cd, afo, ad, eA, av, jz, eW, aT, eV, hn, vB, K, ey, H, hH, ae, af, bc, aRY, aa, yo, A, ND, aW, Y, cr, r, eZ, aq, lo, si, g, pt, t, bz, LB, ck, gk, es, tj, eM, iA, dB, hc, mx, hL, hR, k, Hb, R, m, bt, B, L); #c1(DDR1) c2(XP_011513189) c3(epithelial discoidin domain-containing receptor 1 isoform X1) c4(305118, 305119, 305120, 305121, 305122, 305123, 305124, 305125, 305126, 305127, 305128, 305129, 305130, 305131, 305132, 305133, 305134) c5(305135, 305136, 305137, 305138, 305139, 305140, 305141, 305142, 305143, 305144, 305145, 305146, 305147, 305148, 305149, 305150, 305151, 305152, 305153, 305154, 305155, 305156, 305157, 305158, 305159, 305160, 305161, 305162, 305163, 305164, 305165, 305166) c6(305167, 305168, 305169, 305170, 305171, 305172, 305/73, 305174, 305175, 305/76, 305177, 305178) c7(T, dD, ea, al, jx, l, eX, cl, v, qj, ci, vQ, eq, aoj, zP, Ul, pC, q, o, cd, afo, ad, eA, av, jz, eW, aT, eV, hn, vB, K, ey, H, hH, ae, af, bc, aRY, aa, yo, A, ND, aW, Y, cr, r, eZ, aq, lo, si, g, pt, t, bz, LB, ck, gk, es, tj, eM, iA, dB, hc, mx, hL, hR, k, Hb, R, m, bt, B, L); #c1(DDR1) c2(XP_011513190) c3(epithelial discoidin domain-containing receptor 1 isoform X8) c4(305179) c5(305180, 305181, 305182, 305183, 305184, 305185, 305186, 305187, 305188, 305189, 305190, 305191, 305192, 305193, 305194, 305195, 305196, 305197, 305198, 305199, 305200, 305201) c6(305202, 305203, 305204, 305205, 305206, 305207, 305208, 305209) c7(T, dD, ea, al, jx, l, eX, cl, v, qj, ci, vQ, eq, aoj, zP, Ul, pC, q, o, cd, afo, ad, eA, av, jz, eW, aT, eV, hn, vB, K, ey, H, hH, ae, af, bc, aRY, aa, yo, A, ND, aW, Y, cr, r, eZ, aq, lo, si, g, pt, t, bz, LB, ck, gk, es, tj, eM, iA, dB, hc, mx, hL, hR, k, Hb, R, m, bt, B, L); #c1(DDR1) c2(XP_011545985) c3(epithelial discoidin domain-containing receptor 1 isoform X2) c4(344815, 344816) c5(344817, 344818, 344819, 344820, 344821, 344822, 344823, 344824, 344825) c6(344826) c7(T, dD, ea, al, jx, l, eX, cl, v, qj, ci, vl, eq, aoj, zP, Ul, pC, q, o, cd, afo, ad, eA, av, jz, eW, aT, eV, hn, vB, K, ey, H, hH, ae, af, bc, aRY, aa, yo, A, ND, aW, Y, cr, r, eZ, aq, lo, si, g, pt, t, bz, LB, ck, gk, es, tj, eM, iA, dB, hc, mx, hL, hR, k, Hb, R, m, bt, B, L); #c1(DDR1) c2(XP_011545986) c3(epithelial discoidin domain-containing receptor 1 isoform X3) c4(344827) c5(344828, 344829, 344830, 344831, 344832, 344833, 344834, 344835, 344836) c6( ) c7(T, dD, ea, al, jx, l, eX, cl, v, qj, ci, vQ, eq, aoj, zP, Ul, pC, q, o, cd, afo, ad, eA, av, jz, eW, aT, eV, hn, vB, K, ey, H, hH, ae, af, bc, aRY, aa, yo, A, ND, aW, Y, cr, r, eZ, aq, lo, si, g, pt, t, bz, LB, ck, gk, es, tj, eM, iA, dB, hc, mx, hL, hR, k, Hb, R, m, bt, B, L); #c1(DDR1) c2(XP_011545990) c3(epithelial discoidin domain-containing receptor 1 isoform X4) c4(344837, 344838, 344839) c5(344840) c6( ) c7(T, dD, ea, al, jx, l, eX, cl, v, qj, ci, vQ, eq, aoj, zP, Ul, pC, q, o, cd, afo, ad, eA, av, jz, eW, aT, eV, hn, vB, K, ey, H, hH, ae, af, bc, aRY, aa, yo, A, ND, aW, Y, cr, r, eZ, aq, lo, si, g, pt, t, bz, LB, ck, gk, es, tj, eM, iA, dB, hc, mx, hL, hR, k, Hb, R, m, bt, B, L); #c1(DDR1) c2(XP_011545991) c3 (epithelial discoidin domain-containing receptor 1 isoform X5) c4( ) c5(344841, 344842, 344843) c6( ) c7(T, dD, ea, al, jx, l, eX, cl, v, qj, ci, vQ, eq, aoj, zP, Ul, pC, q, o, cd, afo, ad, eA, av, jz, eW, aT, eV, hn, vB, K, ey, H, hH, ae, af, bc, aRY, aa, yo, A, ND, aW, Y, cr, r, eZ, aq, lo, si, g, pt, t, bz, LB, ck, gk, es, tj, eM, iA, dB, hc, mx, hL, hR, k, Hb, R, m, bt, B, L); #c1(DDR2) c2(XP_011507889) c3(discoidin domain-containing receptor 2 isoform X1) c4(298113, 298114, 298115, 298116) c5(298117, 298118, 298119, 298120, 298121, 298122, 298123, 298124, 298125, 298126, 298127, 298128, 298129, 298130, 298131, 298132) c6( ) c7(T, dD, ea, cz, eq, ci, lR, afo, aT, K, bc, bef, aW, ff, hN, en, t, bz, Qt, pb, beg, eM, Qu, au, QC, y); #c1(DDR2) c2(XP_011507891) c3(discoidin domain-containing receptor 2 isoform X3) c4(298133) c5(298134, 298135, 298136, 298137, 298138, 298139, 298140, 298141, 298142, 298143, 298144, 298145, 298146, 298147, 298148, 298149, 298150, 298151, 298152, 298153, 298154, 298155) c6( ) c7(T, dD, ea, cz, eq, ci, lR, afo, aT, K, bc, bef, aW, ff, hN, en, t, bz, Qt, pb, beg, eM, Qu, au, QC, y); #c1(DDRGK1) c2(NP_076424) c3(DDRGK domain-containing protein 1 precursor) c4(214126, 214127, 214128) c5(214129, 214130, 214131, 214132, 214133, 214134, 214135, 214136, 214137, 214138, 214139, 214140, 214141, 214142, 214143, 214144, 214145, 214146, 214147, 214148) c6( ) c7(F); #c1(DDT) c2(NP_001077861) c3(D-dopachrome decarboxylase) c4(37875, 37876) c5(37877, 37878, 37879, 37880, 37881, 37882, 37883, 37884, 37885, 37886, 37887, 37888, 37889, 37890, 37891, 37892) c6( ) c7(Il, lF, T, es, B, aE, eM, bbC, iu, aW, iA, dV, aT, avB, aq, g, kk, xw, q); #c1(DDTL) c2(NP_001077862) c3(D-dopachrome decarboxylase-like protein) c4(37893) c5(37894, 37895, 37896, 37897, 37898, 37899) c6( ) c7(Il, lF, T, es, B, aE, eM, bbC, iu, aW, iA, dV, aT, avB, aq, kk, xw, q); #c1(DDX1) c2(NP_004930) c3(ATP-dependent RNA helicase DDX1) c4(151036, 151037, 151038) c5(151039, 151040, 151041, 151042, 151043, 151044, 151045, 151046, 151047, 151048, 151049, 151050, 151051, 151052, 151053, 151054, 151055, 151056, 151057, 151058, 151059, 151060) c6( ) c7(T, gP, bP, eR, wu, aW, lB, aAx, wc, aT, n, wm, cm, on); #c1(DDX10) c2(NP_004389) c3(probable ATP-dependent RNA helicase DDX10) c4(146053, 146054) c5(146055, 146056, 146057, 146058, 146059, 146060, 146061, 146062, 146063, 146064, 146065, 146066) c6( ) c7(T, aW, EQ, aT, bw, bt, br); #c1(DDX11) c2(NP_001244074) c3(probable ATP-dependent RNA helicase DDX11 isoform 4) c4(91891, 91892) c5( ) c6( ) c7(T, os, FG, if, oZ, nA, qN, beh, K, cp, Y, CT, g, QK, dr, bN, iP, es, bei, iA, Qu, WI); #c1(DDX11) c2(NP_004390) c3(probable ATP-dependent RNA helicase DDX11 isoform 2) c4(146067) c5(146068, 146069, 146070, 146071, 146072) c6( ) c7(T, os, FG, if, oZ, nA, qN, beh, K, cp, Y, CT, g, QK, dr, bN, iP, es, bei, iA, Qu, WI); #c1(DDX11) c2(NP_085911) c3(probable ATP-dependent RNA helicase DDX11 isoform 1) c4( ) c5(219620, 219621, 219622, 219623, 219624, 219625, 219626, 219627, 219628, 219629, 219630, 219631) c6( ) c7(T, os, FG, if, oZ, nA, qN, beh, K, cp, Y, CT, g, QK, dr, bN, iP, es, bei, iA, Qu, WI); #c1(DDX11) c2(NP_689651) c3(probable ATP-dependent RNA helicase DDX11 isoform 3) c4( ) c5(243466, 243467, 243468, 243469, 243470, 243471, 243472, 243473, 243474, 243475, 243476, 243477, 243478, 243479) c6( ) c7(T, os, FG, if, oZ, nA, qN, beh, K, cp, Y, CT, g, QK, dr, bN, iP, es, bei, iA, Qu, WI); #c1(DDX11) c2(XP_005253388) c3(probable ATP-dependent RNA helicase DDX11 isoform X3) c4(272449) c5(272450, 272451) c6( ) c7(T, os, FG, if, oZ, nA, qN, beh, K, cp, Y, CT, g, QK, dr, bN, iP, es, bei, iA, Qu, WI); #c1(DDX11) c2(XP_005253390) c3(probable ATP-dependent RNA helicase DDX11 isoform X10) c4(272452) c5(272453) c6( ) c7(T, os, FG, if, oZ, nA, qN, beh, K, cp, Y, CT, g, QK, dr, bN, iP, es, bei, iA, Qu, WI); #c1(DDX11) c2(XP_011518897) c3(probable ATP-dependent RNA helicase DDX11 isoform X11) c4(313248) c5(313249) c6( ) c7(T, os, FG, if, oZ, nA, qN, beh, K, cp, Y, CT, g, QK, dr, bN, iP, es, bei, iA, Qu, WI); #c1(DDX11) c2(XP_011518899) c3(probable ATP-dependent RNA helicase DDX11 isoform X15) c4(313250) c5(313251, 313252, 313253, 313254, 313255, 313256, 313257, 313258, 313259, 313260, 313261) c6( ) c7(T, os, FG, if, oZ, nA, qN, beh, K, cp, Y, CT, g, QK, dr, bN, iP, es, bei, iA, Qu, WI); #c1(DDX17) c2(NP_001091974) c3(probable ATP-dependent RNA helicase DDX17 isoform 3) c4(39107, 39108) c5(39109, 39110, 39111, 39112, 39113, 39114, 39115, 39116, 39117, 39118, 39119, 39120) c6( ) c7(T, bE, aW, aT, bB, X); #c1(DDX17) c2(NP_006377) c3(probable ATP-dependent RNA helicase DDX17 isoform 1) c4(164470, 164471, 164472) c5(164473, 164474, 164475, 164476, 164477, 164478, 164479, 164480, 164481, 164482, 164483, 164484, 164485, 164486, 164487, 164488, 164489, 164490) c6( ) c7(T, bE, aW, aT, bB, X); #c1(DDX18) c2(NP_006764) c3(ATP-dependent RNA helicase DDX18) c4(167868, 167869) c5(167870, 167871, 167872, 167873, 167874, 167875, 167876, 167877, 167878, 167879, 167880, 167881) c6( ) c7(bt); #c1(DDX19A) c2(NP_060802) c3(ATP-dependent RNA helicase DDX19A) c4(197906, 197907, 197908) c5(197909, 197910, 197911, 197912, 197913, 197914, 197915, 197916, 197917, 197918, 197919, 197920) c6( ) c7(T, jx, bej, q, eA, bel, ahD, MR, bW, aT, K, lQ, bV, jf, A, lR, aW, Y, eZ, r, lo, pb, c, Jr, U, uo, aYe, y, bek); #c1(DDX19A) c2(XP_005256087) c3(ATP-dependent RNA helicase DDX19A isoform X1) c4(273943) c5(273944, 273945, 273946, 273947, 273948, 273949, 273950, 273951, 273952, 273953, 273954) c6( ) c7(T, jx, bej, q, eA, bel, ahD, MR, bW, aT, K, lQ, bV, jf, A, lR, aW, Y, eZ, r, lo, pb, c, Jr, U, uo, aYe, y, bek); #c1(DDX21) c2(NP_004719) c3(nucleolar RNA helicase 2 isoform 1) c4(149065, 149066) c5(149067, 149068, 149069, 149070, 149071, 149072) c6( ) c7(uo, bem); #c1(DDX21) c2(XP_011538638) c3(nucleolar RNA helicase 2 isoform X1) c4( ) c5(336385, 336386, 336387, 336388, 336389, 336390) c6( ) c7(uo, bem); #c1(DDX25) c2(NP_037396) c3(ATP-dependent RNA helicase DDX25) c4(175252) c5(175253, 175254, 175255, 175256, 175257, 175258) c6( ) c7(pv, gU, ND, ben, afc, vH); #c1(DDX27) c2(NP_060365) c3(probable ATP-dependent RNA helicase DDX27) c4(195612, 195613) c5(195614, 195615, 195616, 195617, 195618, 195619, 195620, 195621, 195622, 195623, 195624, 195625) c6( ) c7(IT); #c1(DDX28) c2(NP_060850) c3(probable ATP-dependent RNA helicase DDX28) c4(198117) c5(198118, 198119, 198120, 198121, 198122, 198123) c6( ) c7(q, r); #c1(DDX31) c2(NP_073616) c3(probable ATP-dependent RNA helicase DDX31 isoform 1) c4(213146, 213147) c5(213148, 213149, 213150, 213151, 213152, 213153, 213154, 213155, 213156, 213157, 213158, 213159, 213160, 213161, 213162, 213163, 213164, 213165) c6( ) c7(fr); #c1(DDX31) c2(NP_619526) c3(probable ATP-dependent RNA helicase DDX31 isoform 2) c4( ) c5(236817, 236818, 236819, 236820, 236821, 236822, 236823) c6( ) c7(fr); #c1(DDX31) c2(XP_005272263) c3(probable ATP-dependent RNA helicase DDX31 isoform X2) c4(283922) c5(283923, 283924, 283925, 283926, 283927, 283928) c6( ) c7(fr); #c1(DDX31) c2(XP_005272264) c3 (probable ATP-dependent RNA helicase DDX31 isoform X3) c4(283929, 283930) c5(283931, 283932, 283933, 283934, 283935, 283936) c6( ) c7(fr); #c1(DDX31) c2(XP_011517223) c3(probable ATP-dependent RNA helicase DDX31 isoform X1) c4(310811) c5( ) c6( ) c7(fr); #c1(DDX39A) c2(NP_005795) c3(ATP-dependent RNA helicase DDX39A) c4(159508, 159509, 159510, 159511) c5(159512, 159513, 159514, 159515, 159516, 159517, 159518, 159519, 159520, 159521, 159522, 159523, 159524, 159525, 159526, 159527, 159528, 159529, 159530, 159531, 159532, 159533, 159534, 159535, 159536, 159537) c6( ) c7(AW, beo, A, aw, uz, gA, dL, aj, iV, y, eL, af, J); #c1(DDX39B) c2(NP_542165) c3(spliceosome RNA helicase DDX39B) c4(231500, 231501, 231502, 231503, 231504, 231505, 231506, 231507, 231508, 231509, 231510, 231511, 231512, 231513, 231514, 231515, 231516, 231517, 231518, 231519, 231520, 231521, 231522, 231523, 231524, 231525, 231526, 231527, 231528, 231529, 231530, 231531) c5(231532, 231533, 231534, 231535, 231536, 231537, 231538, 231539, 231540, 231541, 231542, 231543, 231544, 231545, 231546, 231547, 231548, 231549, 231550, 231551, 231552, 231553, 231554, 231555, 231556, 231557, 231558, 231559, 231560, 231561, 231562, 231563, 231564, 231565, 231566, 231567, 231568, 231569, 231570, 231571, 231572, 231573, 231574, 231575, 231576, 231577, 231578, 231579, 231580, 231581, 231582, 231583, 231584, 231585, 231586, 231587, 231588, 231589, 231590, 231591, 231592, 231593, 231594, 231595, 231596, 231597, 231598, 231599, 231600, 231601, 231602, 231603, 231604, 231605, 231606, 231607, 231608, 231609, 231610, 231611, 231612, 231613, 231614, 231615, 231616, 231617, 231618, 231619, 231620, 231621, 231622, 231623, 231624, 231625, 231626, 231627, 231628, 231629, 231630, 231631, 231632, 231633, 231634, 231635, 231636, 231637, 231638, 231639, 231640, 231641, 231642, 231643, 231644, 231645, 231646, 231647, 231648, 231649, 231650, 231651, 231652, 231653, 231654, 231655, 231656, 231657, 231658, 231659, 231660, 231661, 231662, 231663, 231664, 231665, 231666, 231667, 231668, 231669, 231670, 231671, 231672, 231673, 231674, 231675, 231676, 231677, 231678, 231679, 231680, 231681, 231682, 231683, 231684, 231685, 231686, 231687, 231688, 231689, 231690, 231691, 231692, 231693, 231694, 231695, 231696, 231697, 231698, 231699, 231700, 231701, 231702, 231703, 231704, 231705, 231706, 231707, 231708, 231709, 231710, 231711, 231712, 231713, 231714, 231715, 231716, 231717, 231718, 231719, 231720, 231721, 231722, 231723, 231724, 231725, 231726, 231727, 231728, 231729, 231730, 231731, 231732, 231733, 231734, 231735, 231736, 231737, 231738, 231739, 231740, 231741, 231742, 231743, 231744, 231745, 231746, 231747, 231748, 231749, 231750, 231751, 231752, 231753, 231754, 231755, 231756, 231757, 231758, 231759, 231760, 231761, 231762, 231763, 231764, 231765, 231766, 231767, 231768, 231769, 231770, 231771, 231772, 231773, 231774, 231775, 231776, 231777, 231778, 231779, 231780, 231781, 231782, 231783, 231784, 231785, 231786, 231787, 231788, 231789, 231790, 231791, 231792, 231793, 231794, 231795, 231796, 231797, 231798, 231799, 231800, 231801, 231802, 231803, 231804, 231805, 231806, 231807, 231808, 231809, 231810, 231811, 231812, 231813, 231814, 231815, 231816, 231817, 231818, 231819, 231820, 231821, 231822, 231823, 231824, 231825, 231826, 231827, 231828, 231829, 231830, 231831, 231832, 231833, 231834, 231835, 231836, 231837, 231838, 231839, 231840, 231841, 231842, 231843, 231844, 231845, 231846, 231847, 231848, 231849, 231850, 231851, 231852, 231853, 231854, 231855, 231856, 231857, 231858, 231859, 231860, 231861, 231862, 231863, 231864, 231865, 231866, 231867, 231868, 231869, 231870, 231871, 231872, 231873, 231874, 231875, 231876, 231877, 231878, 231879, 231880, 231881, 231882, 231883, 231884, 231885, 231886, 231887, 231888, 231889, 231890, 231891, 231892, 231893, 231894, 231895, 231896, 231897, 231898, 231899, 231900, 231901, 231902, 231903, 231904, 231905, 231906, 231907, 231908, 231909, 231910, 231911, 231912, 231913, 231914, 231915, 231916, 231917, 231918, 231919, 231920, 231921, 231922, 231923, 231924, 231925, 231926, 231927, 231928, 231929, 231930, 231931, 231932, 231933, 231934, 231935, 231936, 231937, 231938, 231939, 231940, 231941, 231942, 231943, 231944, 231945, 231946, 231947, 231948, 231949, 231950, 231951, 231952, 231953, 231954, 231955, 231956, 231957, 231958, 231959, 231960, 231961, 231962, 231963, 231964, 231965, 231966, 231967, 231968, 231969, 231970, 231971, 231972, 231973, 231974, 231975, 231976, 231977, 231978, 231979, 231980, 231981, 231982, 231983, 231984, 231985, 231986, 231987, 231988, 231989, 231990, 231991, 231992, 231993, 231994, 231995, 231996, 231997, 231998, 231999, 232000, 232001, 232002, 232003, 232004, 232005, 232006, 232007, 232008, 232009, 232010, 232011, 232012, 232013, 232014, 232015, 232016, 232017, 232018, 232019, 232020, 232021, 232022, 232023, 232024, 232025, 232026, 232027, 232028, 232029, 232030, 232031, 232032, 232033, 232034, 232035, 232036, 232037, 232038, 232039, 232040, 232041, 232042, 232043, 232044, 232045, 232046, 232047, 232048, 232049, 232050, 232051, 232052, 232053, 232054, 232055, 232056, 232057, 232058, 232059, 232060, 232061, 232062, 232063, 232064, 232065, 232066, 232067, 232068, 232069, 232070, 232071, 232072, 232073, 232074, 232075, 232076, 232077, 232078, 232079, 232080, 232081, 232082, 232083, 232084, 232085, 232086, 232087, 232088, 232089, 232090, 232091, 232092, 232093, 232094, 232095, 232096, 232097, 232098, 232099, 232100, 232101, 232102, 232103, 232104, 232105, 232106, 232107, 232108, 232109, 232110, 232111, 232112, 232113, 232114, 232115, 232116, 232117, 232118, 232119, 232120, 232121, 232122, 232123, 232124, 232125, 232126, 232127, 232128, 232129, 232130, 232131, 232132, 232133, 232134, 232135, 232136, 232137, 232138, 232139, 232140, 232141) c6( ) c7(bM, aw, gA, aj, V, J, AW, cc, dV, h, hH, af, aiJ, beo, A, aXy, eZ, iV, eE, iQ, Om, uz, y); #c1(DDX3X) c2(NP_001180345) c3(ATP-dependent RNA helicase DDX3X isoform 2) c4( ) c5(73845, 73846, 73847, 73848, 73849, 73850) c6( ) c7(T, jx, hX, av, il, aT, h, ey, K, jf, aW, bep, hl, aq, lo, cl, bz, bP, eM, Uq, aiM, cj, hL, hE, B); #c1(DDX3X) c2(NP_001180346) c3(ATP-dependent RNA helicase DDX3X isoform 3) c4(73851) c5(73852, 73853, 73854, 73855, 73856, 73857) c6( ) c7(T, jx, hX, av, il, aT, h, ey, K, jf, aW, bep, hl, aq, lo, cl, bz, bP, eM, Uq, aiM, cj, hL, HE, B); #c1(DDX3X) c2(NP_001347) c3(ATP-dependent RNA helicase DDX3X isoform 1) c4(117193) c5(117194) c6( ) c7(T, jx, hX, av, il, aT, h, ey, K, jf, aW, bep, hl, aq, lo, cl, bz, bP, eM, Uq, aiM, cj, hL, hE, B); #c1(DDX3X) c2(XP_011542194) c3(ATP-dependent RNA helicase DDX3X isoform X1) c4(340669, 340670) c5(340671, 340672, 340673, 340674, 340675, 340676, 340677, 340678, 340679, 340680, 340681) c6( ) c7(T, jx, hX, av, il, aT, h, ey, K, jf, aW, bep, hl, aq, lo, cl, bz, bP, eM, Uq, aiM, cj, hL, hE, B); #c1(DDX3Y) c2(NP_001289481) c3(ATP-dependent RNA helicase DDX3Y isoform 2) c4(114217, 114218) c5(114219, 114220, 114221, 114222, 114223, 114224, 114225, 114226, 114227, 114228, 114229, 114230) c6( ) c7(beq, gU, ND, wu, eD, wm, Ul, bu); #c1(DDX3Y) c2(NP_004651) c3(ATP-dependent RNA helicase DDX3Y isoform 1) c4(148516) c5(148517, 148518, 148519, 148520, 148521, 148522, 148523, 148524, 148525, 148526) c6( ) c7(beq, gU, ND, wu, eD, wm, Ul, bu); #c1(DDX3Y) c2(XP_011529773) c3(ATP-dependent RNA helicase DDX3Y isoform X2) c4(325456) c5(325457, 325458, 325459, 325460, 325461, 325462) c6( ) c7(beq, gU, ND, wu, eD, wm, UI, bu); #c1(DDX4) c2(NP_001136021) c3(probable ATP-dependent RNA helicase DDX4 isoform 2) c4(54019, 54020) c5(54021, 54022, 54023, 54024, 54025, 54026, 54027, 54028, 54029, 54030, 54031, 54032, 54033, 54034, 54035, 54036) c6( ) c7(Le, gU, ND, vT, wu, eD, Pl, Me, KY, jh, vH, bbs, cm, m, wm, ber, t, KQ); #c1(DDX4) c2(NP_001160005) c3(probable ATP-dependent RNA helicase DDX4 isoform 3) c4(65022) c5(65023, 65024, 65025, 65026, 65027, 65028) c6( ) c7(Le, gU, ND, vT, wu, eD, Pl, Me, KY, jh, vH, bbs, cm, m, wm, ber, t, KQ); #c1(DDX4) c2(NP_00160006) c3(probable ATP-dependent RNA helicase DDX4 isoform 4) c4(65029) c5(65030, 65031, 65032, 65033, 65034, 65035) c6( ) c7(Le, gU, ND, vT, wu, eD, Pl, Me, KY, jh, vH, bbs, cm, m, wm, ber, t, KQ); #c1(DDX4) c2(NP_077726) c3(probable ATP-dependent RNA helicase DDX4 isoform 1) c4(215300, 215301) c5(215302, 215303, 215304, 215305, 215306, 215307, 215308, 215309, 215310, 215311, 215312, 215313) c6( ) c7(Le, gU, ND, vT, wu, eD, Pl, Me, KY, jh, vH, bbs, cm, m, wm, ber, t, KQ); #c1(DDX41) c2(NP_057306) c3(probable ATP-dependent RNA helicase DDX41) c4(190027) c5(190028, 190029, 190030, 190031, 190032, 190033) c6( ) c7(x, bP, Y, fd, h, uz, k, cm); #c1(DDX42) c2(NP_987095) c3(ATP-dependent RNA helicase DDX42) c4(263380, 263381) c5(263382, 263383, 263384, 263385, 263386, 263387, 263388, 263389, 263390, 263391, 263392, 263393, 263394, 263395, 263396, 263397, 263398, 263399, 263400, 263401, 263402, 263403, 263404, 263405) c6( ) c7(jf, Uq); #c1(DDX43) c2(NP_061135) c3(probable ATP-dependent RNA helicase DDX43) c4(199443, 199444) c5(199445, 199446, 199447, 199448, 199449, 199450, 199451, 199452, 199453, 199454, 199455, 199456, 199457, 199458, 199459) c6( ) c7(ck, T, aMa, aAS, lT, iU, Y, ci, bu, bt, bz); #c1(DDX46) c2(NP_001287789) c3(probable ATP-dependent RNA helicase DDX46 isoform 1) c4(112883) c5(112884, 112885, 112886, 112887, 112888, 112889) c6( ) c7(T, jx, bej, q, eA, bel, ahD, MR, bW, aT, K, lQ, bV, jf, A, lR, aW, Y, eZ, r, lo, pb, c, Jr, U, uo, aYe, y, bek); #c1(DDX46) c2(NP_055644) c3(probable ATP-dependent RNA helicase DDX46 isoform 2) c4(182512, 182513) c5(182514, 182515, 182516, 182517, 182518, 182519) c6( ) c7(T, jx, bej, q, eA, bel, ahD, MR, bW, aT, K, lQ, bV, jf, A, lR, aW, Y, eZ, r, lo, pb, c, Jr, U, uo, aYe, y, bek); #c1(DDX46) c2(XP_011542060) c3(probable ATP-dependent RNA helicase DDX46 isoform X2) c4( ) c5(340582, 340583, 340584, 340585, 340586, 340587) c6( ) c7(T, jx, bej, q, eA, bel, ahD, MR, bW, aT, K, lQ, bV, jf, A, lR, aW, Y, eZ, r, lo, pb, c, Jr, U, uo, aYe, y, bek); #c1(DDX5) c2(NP_004387) c3(probable ATP-dependent RNA helicase DDX5) c4(146014, 146015) c5(146016, 146017, 146018, 146019, 146020, 146021, 146022, 146023, 146024, 146025, 146026, 146027, 146028, 146029, 146030, 146031, 146032, 146033) c6( ) c7(dP, gk, T, F, bP, bE, aW, ds, al, iw, aT, bB, fd, hP, eA, t, iK, ae, X, ad, dR); #c1(DDX5) c2(XP_011522758) c3(probable ATP-dependent RNA helicase DDX5 isoform X2) c4(317274) c5( ) c6( ) c7(dP, gk, T, F, bP, bE, aW, ds, al, iw, aT, bB, fd, hP, eA, t, iK, ae, X, ad, dR); #c1(DDX50) c2(NP_076950) c3(ATP-dependent RNA helicase DDX50) c4(214443, 214444) c5(214445, 214446, 214447, 214448, 214449, 214450, 214451, 214452, 214453, 214454, 214455, 214456) c6( ) c7(Z); #c1(DDX51) c2(NP_778236) c3(ATP-dependent RNA helicase DDX51) c4(251484) c5(251485, 251486, 251487, 251488, 251489, 251490) c6( ) c7(x, k); #c1(DDX52) c2(NP_008941) c3(probable ATP-dependent RNA helicase DDX52 isoform 1) c4(169697, 169698) c5(169699, 169700, 169701, 169702, 169703, 169704, 169705, 169706, 169707, 169708, 169709, 169710) c6( ) c7(ae, ad, bX); #c1(DDX53) c2(NP_874358) c3(DEAD box protein 53) c4(256605) c5(256606, 256607, 256608, 256609, 256610, 256611) c6( ) c7(T, os, if, al, aA, bB, Gd, oZ, fr, ci, qN, q, ad, eW, Si, av, F, qp, aT, ae, jf, B, aW, Y, ev, az, hl, ff, r, aFr, aq, aF, aB, en, mo, bu, cl, bN, gk, sS, Dy, iA, bP, bE, Gs, hL, X, cn); #c1(DDX54) c2(NP_001104792) c3(ATP-dependent RNA helicase DDX54 isoform 1) c4(42304) c5(42305, 42306, 42307, 42308, 42309, 42310) c6( ) c7(aq, kl); #c1(DDX54) c2(NP_076977) c3(ATP-dependent RNA helicase DDX54 isoform 2) c4(214557) c5(214558, 214559, 214560, 214561, 214562, 214563) c6( ) c7(aq, kl); #c1(DDX56) c2(NP_001244118) c3(probable ATP-dependent RNA helicase DDX56 isoform 2) c4(91923) c5(91924, 91925, 91926, 91927, 91928, 91929) c6( ) c7(T, d, aiV, Uq, jx, h, j, hE, ae, ad); #c1(DDX56) c2(NP_061955) c3 (probable ATP-dependent RNA helicase DDX56 isoform 1) c4(201293) c5(201294, 201295, 201296, 201297, 201298, 201299) c6( ) c7(T, d, aiV, Uq, jx, h, j, hE, ae, ad); #c1(DDX58) c2(NP_055129) c3(probable ATP-dependent RNA helicase DDX58) c4(179124, 179125, 179126, 179127, 179128, 179129) c5(179130, 179131, 179132, 179133, 179134, 179135, 179136, 179137, 179138, 179139, 179140, 179141, 179142, 179143, 179144, 179145, 179146, 179147, 179148, 179149, 179150, 179151, 179152, 179153, 179154, 179155, 179156, 179157, 179158, 179159, 179160, 179161, 179162, 179163, 179164, 179165) c6( ) c7(gV, T, Dw, fR, if, jx, EA, qN, V, eW, hX, F, hb, Dq, ds, cU, aAn, h, DU, lQ, ey, afu, A, d, bes, Y, apn, cr, eZ, aiT, lo, kp, CQ, iV, t, iK, awi, Bv, Jr, NL, hc, AH, Uq, aiM, m, y); #c1(DDX59) c2(NP_001026895) c3(probable ATP-dependent RNA helicase DDX59) c4(29916, 29917) c5(29918, 29919, 29920, 29921, 29922, 29923) c6( ) c7(bet, ahD, EQ); #c1(DDX59) c2(XP_005245577) c3(probable ATP-dependent RNA helicase DDX59 isoform X3) c4(266732, 266733) c5(266734, 266735, 266736, 266737, 266738, 266739, 266740, 266741, 266742, 266743, 266744, 266745) c6( ) c7(bet, ahD, EQ); #c1(DDX59) c2(XP_005245578) c3(probable ATP-dependent RNA helicase DDX59 isoform X5) c4(266746) c5(266747, 266748, 266749, 266750, 266751, 266752, 266753, 266754, 266755, 266756, 266757, 266758, 266759) c6( ) c7(bet, ahD, EQ); #c1(DDX6) c2(NP_004388) c3(probable ATP-dependent RNA helicase DDX6) c4(146034, 146035) c5(146036, 146037, 146038, 146039, 146040, 146041, 146042, 146043, 146044, 146045, 146046, 146047, 146048, 146049, 146050, 146051, 146052) c6( ) c7(cT, B, fM, aiM, eZ, ci, eA, hF, q, y, bc, dR); #c1(DDX6) c2(XP_011540946) c3(probable ATP-dependent RNA helicase DDX6 isoform X2) c4( ) c5(339471, 339472, 339473, 339474, 339475, 339476) c6( ) c7(cT, B, fM, aiM, eZ, ci, eA, hF, q, y, bc, dR); #c1(DDX60) c2(NP_060101) c3(probable ATP-dependent RNA helicase DDX60) c4(194123, 194124, 194125) c5(194126, 194127, 194128, 194129, 194130, 194131, 194132) c6( ) c7(ds); #c1(DDX60) c2(XP_011530405) c3(probable ATP-dependent RNA helicase DDX60 isoform X1) c4( ) c5(326255, 326256, 326257, 326258, 326259) c6( ) c7(ds); #c1(DDX60) c2(XP_011530406) c3(probable ATP-dependent RNA helicase DDX60 isoform X2) c4( ) c5(326260, 326261, 326262, 326263, 326264, 326265) c6( ) c7(ds); #c1(DEAF1) c2(NP_001280563) c3(deformed epidermal autoregulatory factor 1 homolog isoform b) c4(111525) c5(111526, 111527, 111528, 111529, 111530, 111531, 111532, 111533) c6(111534, 111535) c7(T, A, tb, cA, bO, aj, bS, ww, K, nA, g, he, q, J); #c1(DEAF1) c2(NP_066288) c3(deformed epidermal autoregulatory factor 1 homolog isoform a) c4(206998, 206999, 207000, 207001) c5(207002, 207003, 207004, 207005, 207006, 207007, 207008, 207009, 207010, 207011, 207012, 207013, 207014, 207015, 207016, 207017, 207018, 207019, 207020, 207021, 207022, 207023, 207024, 207025, 207026, 207027, 207028, 207029, 207030, 207031, 207032, 207033, 207034) c6(207035, 207036, 207037, 207038, 207039, 207040, 207041, 207042) c7(T, A, tb, cA, bO, aj, bS, ww, K, nA, g, he, q, J); #c1 (DEC1) c2(XP_011517055) c3(deleted in esophageal cancer 1 isoform X1) c4(310542) c5(310543, 310544, 310545, 310546, 310547, 310548) c6( ) c7(GI, T, ht, aA, hy, ajM, fr, ci, aE, aT, Gw, hu, aW, ff, aq, aF, en, ik, eM, dB, Gu, it, B); #c1(DECR1) c2(NP_001350) c3(2,4-dienoyl-CoA reductase, mitochondrial precursor) c4(117223) c5(117224, 117225, 117226, 117227, 117228, 117229) c6( ) c7(bM, T, qo, pf, ea, e, eb, bB, fd, qj, ci, Tz, azY, wM, aE, dV, K, asC, VF, aa, aW, az, hl, LZ, CT, g, as, cm, bw, y, wE, U, Hb, m, cE, iu, dD, Ol, aw, ez, baD, n, aj, cd, br, ai, ju, h, Cw, Cu, fe, aq, kp, ajz, kT, avF, B, bL, fG, cz, sM, cR, afK, wo, j, C, V, eA, J, eJ, aT, cy, jj, an, ae, af, afC, OV, Y, HL, t, alC, ac, c, hA, wl, wR, CZ, fD, bt, zA, Kd, id, qb, aD, acI, bs, mw, dG, eq, ad, HF, hd, F, nH, afm, sN, ji, yC, vl, sj, d, lR, nU, cH, beu, eZ, gE, iR, bz, dr, qa, ek, LB, bE, eB, MJ, Km, aS, lC, ra, X, us, pC); #c1(DECR1) c2(XP_011515182) c3(2,4-dienoyl-CoA reductase, mitochondrial isoform X1) c4(308009) c5(308010, 308011, 308012, 308013, 308014, 308015) c6( ) c7(bM, T, qo, pf, ea, e, eb, bB, fd, qj, ci, Tz, azY, wM, aE, dV, K, asC, VF, aa, aW, az, hl, LZ, CT, g, as, cm, bw, y, wE, U, Hb, m, cE, iu, dD, Ol, aw, ez, baD, n, aj, cd, br, ai, ju, h, Cw, Cu, fe, aq, kp, ajz, kT, avF, B, bL, fG, cz, sM, cR, afK, wo, j, C, V, eA, J, eJ, aT, cy, jj, an, ae, af, afC, oV, Y, HL, t, alC, ac, c, hA, wO, wR, CZ, fD, bt, zA, Kd, id, qb, aD, acI, bs, mw, dG, eq, ad, HF, hd, F, nH, afm, sN, ji, yC, vl, sj, d, lR, nU, cH, beu, eZ, gE, iR, bz, dr, qa, ek, LB, bE, eB, MJ, Km, aS, lC, ra, X, us, pC); #c1(DEDD) c2(NP_001034801) c3(death effector domain-containing protein) c4(32321, 32322) c5(32323, 32324, 32325, 32326, 32327, 32328, 32329, 32330, 32331, 32332, 32333, 32334, 32335, 32336, 32337, 32338, 32339, 32340, 32341, 32342, 32343, 32344, 32345, 32346, 32347, 32348) c6( ) c7(gx); #c1(DEDD) c2(XP_005245657) c3(death effector domain-containing protein isoform X1) c4(266843) c5(266844, 266845, 266846, 266847, 266848, 266849, 266850, 266851, 266852, 266853) c6( ) c7(gx); #c1(DEF6) c2(NP_071330) c3(differentially expressed in FDCP 6 homolog) c4(211029, 211030, 211031, 211032) c5(211033, 211034, 211035, 211036, 211037, 211038, 211039, 211040, 211041, 211042, 211043, 211044, 211045, 211046, 211047, 211048, 211049, 211050, 211051, 211052, 211053, 211054, 211055, 211056) c6( ) c7(fr, j, eZ); #c1(DEFA1B) c2(NP_001035965) c3(neutrophil defensin 1 isoform 2 preproprotein) c4(33570) c5(33571, 33572, 33573, 33574, 33575, 33576, 33577, 33578, 33579, 33580, 33581, 33582, 33583, 33584, 33585, 33586, 33587) c6(33588, 33589) c7(bM, T, iU, bO, r, K, g, NM, q, afb); #c1(DEFA1B) c2(NP_001289194) c3(neutrophil defensin 1 isoform 1 preproprotein) c4(114123, 114124, 114125, 114126, 114127, 114128, 114129, 114130, 114131, 114132, 114133, 114134) c5(114135, 114136, 114137) c6( ) c7(bM, T, iU, bO, r, K, g, NM, q, afb); #c1(DEFA3) c2(NP_005208) c3(neutrophil defensin 3 preproprotein) c4( ) c5(153994, 153995, 153996, 153997, 153998, 153999) c6(154000) c7(bev, s, afm, mG, aFr, h, ci, C, jj, y, NM); #c1(DEFA3) c2(XP_011533043) c3(neutrophil defensin 3 isoform X1) c4(329240, 329241, 329242, 329243, 329244, 329245, 329246, 329247, 329248, 329249) c5(329250) c6( ) c7(bev, s, afm, mG, aFr, h, ci, C, jj, y, NM); #c1(DEFA4) c2(NP_001916) c3(neutrophil defensin 4 preproprotein) c4(122182, 122183, 122184, 122185, 122186) c5(122187, 122188, 122189, 122190, 122191, 122192) c6( ) c7(eE, pD, afb, h); #c1(DEFA5) c2(NP_066290) c3(defensin-5 preproprotein) c4(207063, 207064, 207065, 207066, 207067, 207068, 207069, 207070, 207071, 207072, 207073, 207074, 207075, 207076, 207077, 207078, 207079, 207080, 207081, 207082, 207083, 207084, 207085, 207086, 207087, 207088, 207089, 207090, 207091, 207092, 207093, 207094, 207095, 207096) c5(207097, 207098, 207099, 207100, 207101, 207102) c6( ) c7(sY, iV, s, fR, aD, az, aFr, h, fe, hp, ey, g, MC, eE, uC, X, ip); #c1(DEFA6) c2(NP_001917) c3(defensin-6 preproprotein) c4(122193, 122194, 122195) c5(122196, 122197, 122198, 122199, 122200, 122201) c6( ) c7(aA, h, fe, aF, fr, iV, eE, X); #c1(DEFB1) c2(NP_005209) c3(beta-defensin 1 preproprotein) c4(154001, 154002, 154003, 154004, 154005, 154006, 154007, 154008, 154009, 154010, 154011, 154012, 154013, 154014, 154015, 154016, 154017, 154018, 154019, 154020, 154021) c5(154022, 154023, 154024, 154025, 154026, 154027) c6( ) c7(bL, bM, sY, bew, pF, ka, fR, T, aD, oe, axd, aee, aA, bS, dG, ct, C, pM, bex, ci, v, fr, asS, ad, Tz, ip, AW, F, afm, apJ, h, bK, wb, pj, bq, ji, ae, bc, pl, aa, le, ls, A, xR, EU, az, bg, cr, ff, dN, fe, Ph, pD, hp, en, aF, Z, eE, uC, iV, aff, ck, ba, es, fT, sS, iA, ki, AH, at, uz, CZ, EY, aDE, ao); #c1(DEFB103B) c2(NP_061131) c3(beta-defensin 103 precursor) c4(199317, 199318, 199319, 199320, 199321, 199322, 199323, 199324, 199325, 199326) c5(199327, 199328, 199329, 199330, 199331, 199332, 199333, 199334, 199335, 199336, 199337, 199338, 199339, 199340, 199341, 199342, 199343, 199344, 199345, 199346, 199347, 199348, 199349, 199350, 199351, 199352, 199353, 199354, 199355, 199356, 199357, 199358, 199359, 199360, 199361, 199362, 199363, 199364, 199365, 199366, 199367, 199368, 199369, 199370, 199371, 199372, 199373, 199374, 199375, 199376, 199377, 199378, 199379, 199380, 199381, 199382, 199383, 199384, 199385, 199386, 199387, 199388, 199389, 199390, 199391, 199392, 199393, 199394, 199395, 199396, 199397, 199398, 199399, 199400, 199401, 199402, 199403, 199404, 199405, 199406, 199407, 199408, 199409, 199410, 199411, 199412, 199413, 199414, 199415, 199416, 199417, 199418, 199419, 199420, 199421, 199422, 199423, 199424) c6(199425, 199426, 199427) c7(oO, T, Ht, fR, aD, bB, ds, Dn, pj, le, xR, ff, lo, hp, en, iV, eE, ik, bE, bas, CZ, pk); #c1(DEFB104B) c2(NP_001035792) c3(beta-defensin 104 precursor) c4(33094, 33095, 33096, 33097, 33098) c5(33099, 33100, 33101, 33102, 33103, 33104, 33105, 33106, 33107, 33108, 33109) c6(33110, 33111) c7(bE, if, cr, eZ, h, uz, hv, wb, ji, qN, iV, ae, ao, ad, aa); #c1(DEFB105A) c2(NP_689463) c3(beta-defensin 105 precursor) c4(242768, 242769, 242770, 242771, 242772, 242773, 242774, 242775) c5(242776, 242777, 242778, 242779, 242780, 242781, 242782, 242783, 242784, 242785, 242786, 242787) c6(242788, 242789) c7(h); #c1(DEFB106A) c2(NP_689464) c3(beta-defensin 106 precursor) c4(242790, 242791, 242792, 242793, 242794, 242795, 242796) c5(242797, 242798, 242799, 242800, 242801, 242802, 242803, 242804, 242805, 242806, 242807, 242808) c6(242809, 242810) c7(h); #c1(DEFB108B) c2(NP_001002035) c3(beta-defensin 108B precursor) c4(19422, 19423, 19424, 19425) c5(19426, 19427, 19428, 19429, 19430, 19431, 19432) c6(19433) c7(h); #c1(DEFB112) c2(NP_001032587) c3(beta-defensin (12) c4(31388) c5(31389, 31390, 31391, 31392, 31393, 31394, 31395, 31396) c6(31397, 31398) c7(fq, c); #c1(DEFB125) c2(NP_697020) c3(beta-defensin (25 preproprotein) c4(245710, 245711, 245712, 245713, 245714, 245715, 245716, 245717) c5(245718, 245719, 245720, 245721, 245722, 245723, 245724, 245725) c6(245726, 245727) c7(aw); #c1(DEFB126) c2(NP_112193) c3(beta-defensin 126 preproprotein) c4(220731, 220732, 220733, 220734, 220735, 220736) c5(220737, 220738, 220739, 220740, 220741, 220742, 220743, 220744) c6(220745, 220746) c7(AV, iW, d); #c1(DEFB4A) c2(NP_004933) c3(beta-defensin 4A precursor) c4(151068, 151069, 151070, 151071, 151072, 151073, 151074, 151075, 151076, 151077, 151078, 151079, 151080, 151081, 151082, 151083, 151084, 151085, 151086, 151087, 151088, 151089, 151090, 151091, 151092, 151093, 151094, 151095, 151096, 151097, 151098, 151099, 151100, 151101, 151102, 151103, 151104, 151105, 151106, 151107, 151108, 151109, 151110, 151111, 151112, 151113, 151114, 151115, 151116, 151117, 151118, 151119, 151120, 151121, 151122, 151123, 151124, 151125, 151126, 151127, 151128, 151129, 151130, 151131) c5(151132, 151133, 151134, 151135, 151136, 151137, 151138, 151139, 151140, 151141, 151142, 151143, 151144, 151145, 151146, 151147, 151148, 151149, 151150, 151151, 151152, 151153, 151154, 151155, 151156, 151157, 151158, 151159, 151160, 151161, 151162, 151163, 151164, 151165, 151166, 151167, 151168, 151169, 151170, 151171, 151172, 151173, 151174, 151175, 151176, 151177, 151178, 151179, 151180, 151181, 151182, 151183, 151184, 151185, 151186, 151187, 151188, 151189, 151190, 151191, 151192, 151193, 151194, 151195, 151196, 151197, 151198, 151199, 151200, 151201, 151202, 151203, 151204, 151205, 151206, 151207, 151208, 151209, 151210, 151211, 151212, 151213, 151214, 151215, 151216, 151217, 151218, 151219, 151220, 151221, 151222, 151223, 151224, 151225, 151226, 151227, 151228, 151229, 151230) c6(151231, 151232, 151233) c7(oO, fR, tp, aD, if, wh, rl, aA, C, qN, Za, q, asS, Tz, wa, AK, F, bey, rS, dV, aT, h, dL, bK, lQ, Dn, wb, oi, pj, eL, bc, ao, pi, ls, xR, ff, Zf, EU, bez, bg, cr, r, rM, dN, hN, aFr, eZ, aEW, en, iV, eE, fe, axp, pa, ac, JM, wz, at, uz, wR, CZ, fD, hv); #c1(DEGS1) c2(NP_003667) c3(sphingolipid delta(4)-desaturase DESI) c4(140056, 140057) c5(140058, 140059, 140060, 140061, 140062, 140063, 140064, 140065, 140066, 140067, 140068, 140069, 140070, 140071, 140072, 140073) c6( ) c7(ob, zs, hD, aj, ey, hu, J, ad, cp); #c1(DEGS2) c2(NP_996801) c3(sphingolipid delta(4)-desaturase/C4-hydroxylase DES2) c4(264576) c5(264577, 264578, 264579, 264580, 264581, 264582) c6( ) c7(cF); #c1(DEK) c2(NP_001128181) c3(protein DEK isoform 2) c4(49685) c5(49686, 49687, 49688, 49689, 49690, 49691) c6( ) c7(GI, T, oe, oA, bB, cl, ci, o, lg, yt, aT, h, dL, K, eL, je, iU, aW, Y, ff, eH, lo, en, eF, bz, ck, iM, bP, bE, nG, cj, np, bC, oQ, y, bt, X); #c1(DEK) c2(NP_003463) c3(protein DEK isoform 1) c4(138540) c5(138541, 138542, 138543, 138544, 138545, 138546) c6( ) c7(GI, T, oe, oA, bB, cl, ci, o, lg, yt, aT, h, dL, K, eL, je, iU, aW, Y, ff, eH, lo, en, eF, bz, ck, iM, bP, bE, nG, cj, np, bC, oQ, y, bt, X); #c1(DENND1A) c2(NP_065997) c3(DENN domain-containing protein (A isoform 1) c4(206701) c5(206702, 206703, 206704, 206705, 206706, 206707) c6( ) c7(Na, kl, zX, beA, aZ, Nc, j, cl); #c1(DENND1A) c2(NP_079096) c3(DENN domain-containing protein 1A isoform 2) c4(217009) c5(217010, 217011, 217012, 217013, 217014, 217015) c6( ) c7(Na, kl, zX, beA, aZ, Nc, j, cl); #c1(DENND1A) c2(XP_005252170) c3(DENN domain-containing protein 1A isoform X10) c4(271586) c5(271587, 271588, 271589, 271590, 271591, 271592) c6( ) c7(Na, kl, zX, beA, aZ, Nc, j, c1); #c1(DENND1A) c2(XP_006717258) c3(DENN domain-containing protein IA isoform X3) c4(290827, 290828) c5(290829, 290830, 290831, 290832, 290833, 290834, 290835, 290836, 290837, 290838, 290839, 290840) c6( ) c7(Na, kl, zX, beA, aZ, Nc, j, cl); #c1(DENND1A) c2(XP_011517186) c3(DENN domain-containing protein (A isoform X5) c4(310780) c5(310781, 310782, 310783, 310784, 310785, 310786) c6( ) c7(Na, kl, zX, beA, aZ, Nc, j, cl); #c1(DENND1B) c2(NP_001287787) c3(DENN domain-containing protein 1B isoform 5) c4(112872) c5(112873, 112874, 112875, 112876, 112877, 112878, 112879, 112880, 112881, 112882) c6( ) c7(bM, v, j, bc, pM); #c1(DENND1B) c2(NP_659414) c3(DENN domain-containing protein IB isoform 2) c4(239785) c5(239786, 239787, 239788, 239789, 239790, 239791) c6( ) c7(bM, v, j, bc, pM); #c1(DENND1B) c2(XP_005244988) c3(DENN domain-containing protein 18 isoform X5) c4(266185) c5(266186, 266187, 266188, 266189, 266190, 266191) c6( ) c7(bM, v, j, bc, pM); #c1(DENND1B) c2(XP_006711257) c3(DENN domain-containing protein 18 isoform X8) c4(285823) c5(285824, 285825, 285826, 285827, 285828, 285829) c6( ) c7(bM, v, j, bc, pM); #c1(DENND1B) c2(XP_011507550) c3(DENN domain-containing protein 18 isoform X3) c4(297477) c5(297478, 297479, 297480, 297481, 297482, 297483) c6( ) c7(bM, v, j, bc, pM); #c1(DENND2A) c2(NP_056504) c3(DENN domain-containing protein 2A) c4(187805, 187806, 187807) c5(187808, 187809, 187810, 187811, 187812, 187813, 187814, 187815, 187816, 187817, 187818, 187819, 187820, 187821) c6( ) c7(ajP, o); #c1(DENND2A) c2(XP_005250036) c3(DENN domain-containing protein ZA isoform X3) c4( ) c5(270041, 270042, 270043, 270044, 270045, 270046, 270047, 270048, 270049, 270050, 270051, 270052, 270053, 270054, 270055, 270056, 270057, 270058) c6( ) c7(ajP, oQ); #c1(DENND2A) c2(XP_011514356) c3(DENN domain-containing protein 2A isoform X2) c4(307056) c5( ) c6( ) c7(ajP, oQ); #c1(DENND4A) c2(NP_001138295) c3(C-myc promoter-binding protein isoform 1) c4(56135) c5(56136, 56137, 56138, 56139, 56140, 56141) c6( ) c7(gk, hb, oe, al, fd, ae, ad); #c1(DENND4A) c2(NP_005839) c3(C-myc promoter-binding protein isoform 2) c4(159792) c5(159793, 159794, 159795, 159796, 159797, 159798) c6( ) c7(gk, hb, oe, al, fd, ae, ad); #c1(DENND4A) c2(XP_005254179) c3(C-myc promoter-binding protein isoform X5) c4( ) c5(273049) c6( ) c7(gk, hb, oe, al, fd, ae, ad); #c1(DENR) c2(NP_003668) c3(density-regulated protein) c4(140074) c5(140075, 140076, 140077, 140078, 140079, 140080) c6( ) c7(ID, ly, wg, c, Ae, dc, U, cy, uY, dG, K, an, lx); #c1(DEPDC1B) c2(NP_001138680) c3(DEP domain-containing protein 18 isoform 2) c4(57391, 57392) c5(57393, 57394, 57395, 57396, 57397, 57398, 57399, 57400, 57401, 57402, 57403, 57404) c6( ) c7(aS, bd); #c1(DEPDC1B) c2(NP_060839) c3(DEP domain-containing protein (B isoform 1) c4(198078) c5(198079, 198080, 198081, 198082, 198083, 198084) c6( ) c7(aS, bd); #c1(DEPDC5) c2(NP_001007189) c3(DEP domain-containing protein 5 isoform 2) c4(22545) c5(22546, 22547, 22548, 22549, 22550, 22551, 22552, 22553, 22554, 22555, 22556, 22557) c6( ) c7(F, FB, ha, beB, hl, lr, akf, B, aNW, hX); #c1(DEPDC5) c2(NP_001129501) c3(DEP domain-containing protein 5 isoform 3) c4(51710) c5(51711, 51712, 51713, 51714, 51715, 51716) c6( ) c7(F, FB, ha, beB, hl, lr, akf, B, aNW, hX); #c1(DEPDC5) c2(NP_001229825) c3(DEP domain-containing protein 5 isoform 4) c4(85349) c5(85350, 85351, 85352, 85353, 85354, 85355) c6( ) c7(F, FB, ha, beB, hl, lr, akf, B, aNW, hX); #c1(DEPDC5) c2(NP_001229826) c3(DEP domain-containing protein 5 isoform 5) c4(85356, 85357, 85358) c5(85359, 85360, 85361, 85362, 85363, 85364, 85365, 85366, 85367, 85368, 85369, 85370, 85371, 85372, 85373, 85374, 85375, 85376) c6( ) c7(F, FB, ha, beB, hl, lr, akf, B, aNW, hX); #c1(DEPDC5) c2(NP_055477) c3(DEP domain-containing protein 5 isoform 1) c4(181359) c5(181360, 181361, 181362, 181363, 181364, 181365, 181366, 181367, 181368, 181369) c6( ) c7(F, FB, ha, beB, hl, lr, akf, B, aNW, hX); #c1(DEPDC5) c2(XP_011528860) c3(DEP domain-containing protein 5 isoform X5) c4(324249, 324250) c5(324251, 324252, 324253, 324254, 324255, 324256) c6( ) c7(F, FB, ha, beB, hl, lr, akf, B, aNW, hX); #c1(DEPDC5) c2(XP_011528862) c3(DEP domain-containing protein 5 isoform X6) c4(324257, 324258) c5(324259, 324260, 324261, 324262, 324263, 324264) c6( ) c7(F, FB, ha, beB, hl, lr, akf, B, aNW, hX); #c1(DEPDC5) c2(XP_011528871) c3(DEP domain-containing protein 5 isoform X14) c4( ) c5(324265, 324266, 324267, 324268, 324269, 324270) c6( ) c7(F, FB, ha, beB, hl, lr, akf, B, aNW, hX); #c1(DEPTOR) c2(NP_073620) c3(DEP domain-containing mTOR-interacting protein isoform 1) c4(213173) c5(213174, 213175, 213176, 213177, 213178, 213179) c6( ) c7(T, K); #c1(DERA) c2(NP_057038) c3(deoxyribose-phosphate aldolase isoform 1) c4(188592, 188593) c5(188594, 188595, 188596, 188597, 188598, 188599, 188600, 188601, 188602, 188603, 188604, 188605) c6( ) c7(dy); #c1(DERL1) c2(NP_001128143) c3(derlin-1 isoform b) c4(49635) c5(49636, 49637, 49638, 49639, 49640, 49641) c6( ) c7(T, Uc, WA, qx, bs, fd, amj, oY, ci, AO, Fc, br, x, db, aT, gs, dz, hn, beD, qt, aW, aq, pm, Fu, bw, bz, ck, beC, pc, c, eM, pg, k, bt); #c1(DERL1) c2(NP_077271) c3(derlin-1 isoform a) c4(214854, 214855) c5(214856, 214857, 214858, 214859, 214860, 214861) c6( ) c7(T, Uc, WA, qx, bs, fd, amj, oY, ci, AO, Fc, br, x, db, aT, gs, dz, hn, beD, qt, aW, aq, pm, Fu, bw, bz, ck, beC, pc, c, eM, pg, k, bt); #c1(DERL1) c2(XP_011515601) c3(derlin-1 isoform X1) c4( ) c5(308443, 308444, 308445, 308446, 308447, 308448) c6( ) c7(T, Uc, WA, qx, bs, fd, amj, DY, ci, AO, Fc, br, x, db, aT, gs, dz, hn, beD, qt, aW, aq, pm, Fu, bw, bz, ck, beC, pc, c, eM, pg, k, bt); #c1(DERL2) c2(NP_057125) c3(derlin-2 isoform a) c4(189010) c5(189011, 189012, 189013, 189014, 189015, 189016, 189017, 189018) c6(189019, 189020) c7(ck, bv, EW, T, Fp, iD, fR, beE, Pl, jh, pg, aF); #c1(DERL3) c2(NP_001002862) c3(derlin-3 isoform 2) c4(19679) c5(19680, 19681, 19682, 19683, 19684, 19685, 19686) c6( ) c7(es, T, ac, iA, RX, DA, jh, bw, fr, Kg, iJ, avK); #c1(DERL3) c2(NP_001129223) c3(derlin-3 isoform 1) c4(51250, 51251) c5(51252, 51253, 51254, 51255, 51256, 51257) c6( ) c7(es, T, ac, iA, RX, oA, jh, bw, fr, Kg, iJ, avK); #c1(DERL3) c2(NP_940842) c3(derlin-3 isoform 3) c4( ) c5(259934, 259935, 259936, 259937, 259938) c6( ) c7(es, T, ac, iA, RX, oA, jh, bw, fr, Kg, iJ, avK); #c1(DES) c2(NP_001918) c3(desmin) c4(122202) c5(122203, 122204, 122205, 122206, (22207, 122208, 122209) c6( ) c7(T, hV, bB, lR, ci, Hr, bv, K, li, z, aW, Ls, as, cm, wK, wE, vA, mx, bel, Uc, Am, aw, fr, asF, nA, zP, aHZ, cd, cc, Ak, FN, bl, agW, cY, AX, A, kj, Ad, aMH, mo, aYo, alj, op, eD, aWn, va, bD, arO, B, wL, fG, aA, aTl, j, UX, pd, q, er, eA, hW, Ws, aT, adm, aWs, qk, Dr, Y, la, r, en, pb, ck, beH, beJ, c, aXe, hc, km, aof, beG, Fc, hX, Dq, oj, vc, fb, Yi, anR, d, xz, xs, ff, beF, dr, bE, gZ, X); #c1(DESI2) c2(NP_001284675) c3(desumoylating isopeptidase 2 isoform 2) c4(112295) c5(112296, 112297, 112298, 112299, 112300, 112301) c6( ) c7(bE, t, DA, T, m); #c1(DESI2) c2(NP_057160) c3(desumoylating isopeptidase 2 isoform 1) c4(189208) c5(189209, 189210, 189211, 189212, 189213, 189214) c6( ) c7(bE, t, DA, T, m); #c1(DESI2) c2(XP_011542505) c3(desumoylating isopeptidase 2 isoform X1) c4(341052) c5( ) c6( ) c7(bE, t, DA, T, m); #c1(DESI2) c2(XP_011542510) c3(desumoylating isopeptidase 2 isoform X6) c4( ) c5(341053, 341054, 341055, 341056, 341057, 341058) c6( ) c7(bE, t, DA, T, m); #c1(DEX1) c2(NP_054734) c3(dexamethasone-induced protein) c4(177465) c5(177466, 177467, 177468, 177469, 177470, 177471) c6( ) c7(eV, V, A, eX, bt); #c1(DEX1) c2(XP_011520757) c3(dexamethasone-induced protein isoform X1) c4( ) c5(315764) c6( ) c7(eV, V, A, eX, bt); #c1(DFFB) c2(XP_011539165) c3(DNA fragmentation factor subunit beta isoform X1) c4( ) c5(337278, 337279, 337280, 337281, 337282, 337283, 337284, 337285, 337286, 337287) c6( ) c7(bL, T, dD, ZV, Zz, ea, iW, e, aw, cz, sM, vr, bS, j, ct, eq, il, Fc, q, ad, av, ec, rS, aT, h, j, de, eN, hm, bf, cW, aW, Zt, r, aq, vs, Z, cm, ac, nB, wE, aka, vb, hE, bt, B); #c1(DFFB) c2(XP_011539166) c3(DNA fragmentation factor subunit beta isoform X2) c4(337288) c5(337289, 337290, 337291, 337292, 337293, 337294, 337295, 337296, 337297, 337298) c6( ) c7(bL, T, dD, ZV, Zz, ea, iW, e, aw, cz, sM, vr, bS, j, ct, eq, il, Fc, q, ad, av, ec, rS, aT, h, j, dQ, eN, hm, bf, cW, aW, Zt, r, aq, vs, Z, cm, ac, nB, wE, aka, vb, hE, bt, B); #c1(DFFB) c2(XP_011539167) c3(DNA fragmentation factor subunit beta isoform X3) c4( ) c5(337299, 337300, 337301, 337302, 337303, 337304, 337305, 337306, 337307, 337308, 337309, 337310, 337311, 337312, 337313, 337314, 337315, 337316, 337317, 337318, 337319, 337320) c6( ) c7(bL, T, dD, ZV, Zz, ea, iW, e, aw, cz, sM, vr, bS, j, ct, eq, il, Fc, q, ad, av, ec, rS, aT, h, j, dQ, eN, hm, bf, cW, aW, Zt, r, aq, vs, Z, cm, ac, nB, wE, aka, vb, hE, bt, B); #c1(DFNA5) c2(NP_001120925) c3 (non-syndromic hearing impairment protein 5 isoform a) c4(44916, 44917) c5(44918, 44919, 44920, 44921, 44922, 44923, 44924, 44925, 44926, 44927, 44928, 44929) c6( ) c7(T, bek, bE, aW, Y, mG, aT, r, aA, bB, aF, K, Bh, q); #c1(DFNA5) c2(NP_001120926) c3(non-syndromic hearing impairment protein 5 isoform b) c4( ) c5(44930, 44931, 44932, 44933, 44934, 44935, 44936, 44937) c6( ) c7(T, bek, bE, aW, Y, mG, aT, r, aA, bB, aF, K, Bh, q); #c1(DFNB31) c2(NP_056219) c3(whirlin isoform 1) c4(186467, 186468, 186469, 186470) c5(186471, 186472, 186473, 186474, 186475, 186476, 186477, 186478, 186479, 186480, 186481, 186482, 186483, 186484, 186485, 186486, 186487, 186488) c6( ) c7(cz, bB, mS, oc, q, Na, beM, mW, bel, FQ, Nc, bk, mY, mV, mU, r, nC, mZ, mx, bE, mG, alA, nw, Bh, mT); #c1(DFNB31) c2(XP_011516796) c3(whirlin isoform X10) c4( ) c5(310237, 310238, 310239, 310240, 310241, 310242) c6( ) c7(cz, bB, mS, oc, q, Na, beM, mW, bel, FQ, Nc, bK, mY, mV, mU, r, nC, mZ, mX, bE, mG, alA, nw, Bh, mT); #c1(DFNB59) c2(NP_001036167) c3 (pejvakin) c4(34008) c5(34009, 34010, 34011, 34012, 34013, 34014, 34015, 34016, 34017, 34018) c6( ) c7(Bh, bel, beN); #c1(DGAT1) c2(NP_036211) c3(diacylglycerol O-acyltransferase 1) c4(172389) c5(172390, 172391, 172392, 172393, 172394, 172395) c6( ) c7(bL, af, oe, jx, fc, dq, v, cP, cd, bJ); #c1(DGAT2) c2(NP_001240820) c3(diacylglycerol O-acyltransferase 2 isoform 2) c4(88874) c5(88875, 88876, 88877, 88878, 88879, 88880) c6( ) c7(v, fc, hX); #c1(DGAT2) c2(NP_115953) c3(diacylglycerol O-acyltransferase 2 isoform 1) c4(224903) c5(224904, 224905, 224906, 224907, 224908, 224909) c6( ) c7(v, fc, hX); #c1(DGCR2) c2(NP_001167004) c3(integral membrane protein DGCR2/IDD isoform 2 precursor) c4(68813) c5(68814, 68815, 68816, 68817, 68818, 68819) c6( ) c7(agd, A, RX, aw, bD, aj, aKH, beP, NI, cm, bc, J); #c1(DGCR2) c2(NP_001167005) c3(integral membrane protein DGCR2/IDD isoform 3 precursor) c4( ) c5(68820) c6( ) c7(agd, A, RX, aw, bD, aj, aKH, beP, NI, cm, bc, J); #c1(DGCR2) c2(NP_001171710) c3(integral membrane protein DGCR2/IDD isoform 4 precursor) c4(71375) c5(71376, 71377, 71378, 71379, 71380) c6( ) c7(agd, A, RX, aw, bD, aj, aKH, beP, NI, cm, bc, J); #c1(DGCR2) c2(NP_005128) c3(integral membrane protein DGCR2/IDD isoform 1 precursor) c4(153085, 153086) c5(153087, 153088, 153089, 153090, 153091, 153092, 153093, 153094, 153095, 153096, 153097, 153098) c6( ) c7(agd, A, RX, aw, bD, aj, aKH, beP, NI, cm, bc, J); #c1(DGCRGL) c2(NP_150282) c3(protein DGCR6L) c4(228035) c5(228036, 228037, 228038, 228039, 228040, 228041) c6( ) c7(RX, agd, bN, hq); #c1(DGCR8) c2(NP_001177255) c3(microprocessor complex subunit DGCR8 isoform 2) c4(72552) c5(72553, 72554, 72555, 72556, 72557, 72558, 72559, 72560, 72561, 72562, 72563, 72564, 72565, 72566, 72567, 72568) c6( ) c7(es, BN, aW, yk, RX, aT, hy, fr, aan, he, agY, ne); #c1(DGCR8) c2(NP_073557) c3(microprocessor complex subunit DGCR8 isoform 1) c4(212845, 212846) c5(212847, 212848, 212849, 212850, 212851, 212852) c6( ) c7(es, BN, aW, yk, RX, aT, hy, fr, aan, he, agY, ne); #c1(DGKA) c2(NP_958853) c3(diacylglycerol kinase alpha) c4(262298, 262299, 262300) c5(262301, 262302, 262303, 262304, 262305, 262306, 262307, 262308, 262309, 262310, 262311, 262312, 262313, 262314, 262315, 262316, 262317, 262318, 262319, 262320, 262321, 262322, 262323, 262324, 262325, 262326, 262327, 262328, 262329, 262330, 262331, 262332, 262333, 262334, 262335, 262336, 262337, 262338, 262339, 262340, 262341, 262342, 262343, 262344, 262345) c6( ) c7(T, d, Y, hl, n, nw, cl, B, Tz); #c1(DGKA) c2(XP_005268745) c3(diacylglycerol kinase alpha isoform X2) c4(282083) c5(282084, 282085, 282086, 282087, 282088, 282089, 282090, 282091, 282092, 282093, 282094, 282095, 282096, 282097, 282098, 282099, 282100, 282101, 282102) c6( ) c7(T, d, Y, hL, n, nw, cl, B, Tz); #c1(DGKA) c2(XP_011536292) c3(diacylglycerol kinase alpha isoform X3) c4(333627, 333628, 333629, 333630, 333631) c5(333632, 333633, 333634, 333635, 333636, 333637, 333638, 333639, 333640) c6( ) c7(T, d, Y, hL, n, nw, cl, B, Tz); #c1(DGKB) c2(NP_004071) c3(diacylglycerol kinase beta isoform 1) c4(143292, 143293) c5(143294, 143295, 143296, 143297, 143298, 143299, 143300, 143301, 143302, 143303, 143304, 143305, 143306, 143307, 143308, 143309) c6( ) c7(bL, gk, cF, aw, al, k, aoz); #c1(DGKB) c2(NP_663733) c3(diacylglycerol kinase beta isoform 2) c4(241470) c5(241471, 241472, 241473, 241474, 241475, 241476) c6( ) c7(bL, gk, cF, aw, al, k, aoz); #c1(DGKB) c2(XP_005249686) c3(diacylglycerol kinase beta isoform X2) c4(269777, 269778) c5(269779, 269780, 269781, 269782, 269783, 269784) c6( ) c7(bL, gk, cF, aw, al, k, aoz); #c1(DGKB) c2(XP_005249688) c3(diacylglycerol kinase beta isoform X6) c4(269785) c5(269786, 269787, 269788, 269789, 269790, 269791) c6( ) c7(bL, gk, cF, aw, al, k, aoz); #c1(DGKB) c2(XP_011513458) c3(diacylglycerol kinase beta isoform X4) c4(305711) c5(305712, 305713, 305714, 305715, 305716, 3057(7) c6( ) c7(bL, gk, cF, aw, al, k, aoz); #c1(DGKB) c2(XP_011513462) c3(diacylglycerol kinase beta isoform X8) c4( ) c5(305718, 305719, 305720, 305721, 305722, 305723) c6( ) c7(bL, gk, cF, aw, al, k, aoz); #c1(DGKD) c2(NP_003639) c3(diacylglycerol kinase delta isoform 1) c4(139809) c5(139810, 139811, 139812, 139813, 139814, 139815) c6( ) c7(bL, T, beR, beQ); #c1(DGKD) c2(NP_690618) c3(diacylglycerol kinase delta isoform 2) c4(244930) c5(244931, 244932, 244933, 244934, 244935, 244936) c6( ) c7(bL, T, beR, beQ); #c1(DGKE) c2(NP_003638) c3(diacylglycerol kinase epsilon) c4( ) c5(139804, 139805, 139806, 139807, 139808) c6( ) c7(bL, gk, al, beS, vl, aoz); #c1(DGKE) c2(XP_011523696) c3(diacylglycerol kinase epsilon isoform X1) c4(318173) c5(318174) c6( ) c7(bL, gk, al, beS, vl, aoz); #c1(DGKG) c2(NP_001074213) c3(diacylglycerol kinase gamma isoform 2) c4(36987) c5(36988, 36989, 36990, 36991, 36992, 36993) c6( ) c7(bL, fq, v, oQ); #c1(DGKG) c2(NP_001074214) c3(diacylglycerol kinase gamma isoform 3) c4(36994, 36995, 36996) c5(36997, 36998, 36999, 37000, 37001, 37002, 37003, 37004, 37005, 37006, 37007, 37008, 37009, 37010, 37011, 37012, 37013, 37014) c6( ) c7(bL, fq, v, oQ); #c1(DGKG) c2(NP_001337) c3(diacylglycerol kinase gamma isoform 1) c4(1/7021) c5(117022, 117023, 117024, 117025, 117026, 117027) c6( ) c7(bL, fq, v, oQ); #c1(DGKH) c2(NP_001191433) c3(diacylglycerol kinase eta isoform 1) c4(81497) c5(81498, 81499, 81500, 81501, 81502, 81503, 81504, 81505, 81506, 81507) c6( ) c7(uJ, bK, af); #c1(DGKH) c2(NP_001191434) c3(diacylglycerol kinase eta isoform 3) c4(81508) c5(81509, 81510, 81511, 81512, 81513) c6( ) c7(uJ, bk, af); #c1(DGKH) c2(NP_001191435) c3(diacylglycerol kinase eta isoform 4) c4( ) c5(81514, 81515, 81516, 81517, 81518) c6( ) c7(uJ, bk, af); #c1(DGKH) c2(NP_001284358) c3(diacylglycerol kinase eta isoform 5) c4(111764) c5(11/765, 111766, 111767, 111768, 111769, 111770) c6( ) c7(uJ, bK, af); #c1(DGKH) c2(NP_821077) c3(diacylglycerol kinase eta isoform 2) c4(253198, 253199) c5(253200, 253201, 253202, 253203, 253204, 253205, 253206, 253207) c6( ) c7(uJ, bK, af); #c1(DGKI) c2(NP_004708) c3(diacylglycerol kinase iota) c4(148989, 148990) c5(148991, 148992, 148993, 148994, 148995, 148996) c6( ) c7(cm, fq, il, yA); #c1(DGKI) c2(XP_006716235) c3(diacylglycerol kinase iota isoform X1) c4( ) c5(290192, 290193, 290194, 290195, 290196, 290197) c6( ) c7(cm, fq, iQ, yA); #c1(DGKI) c2(XP_011514980) c3 (diacylglycerol kinase iota isoform X3) c4(307787) c5(307788, 307789, 307790, 307791, 307792, 307793) c6( ) c7(cm, fq, iQ, yA); #c1(DGKK) c2(NP_001013764) c3(diacylglycerol kinase kappa) c4(25751) c5(25752, 25753, 25754, 25755, 25756, 25757, 25758) c6( ) c7(beU, beT); #c1(DGKQ) c2(NP_001338) c3(diacylglycerol kinase theta) c4(117028) c5(117029, 117030, 117031, 117032, 117033, 117034) c6( ) c7(nw, fN, an); #c1(DGKQ) c2(XP_011511717) c3(diacylglycerol kinase theta isoform X4) c4( ) c5(302439) c6( ) c7(nw, fN, an); #c1(DGKZ) c2(NP_001099010) c3 (diacylglycerol kinase zeta isoform 4) c4(41641) c5(41642, 41643, 41644, 41645, 41646, 41647) c6( ) c7(bB, bE, T, PX, aw); #c1(DGKZ) c2(NP_001186195) c3(diacylglycerol kinase zeta isoform 5) c4(77400) c5(77401, 77402, 77403, 77404, 77405, 77406) c6( ) c7(bB, bE, T, PX, aw); #c1(DGKZ) c2(NP_001186196) c3(diacylglycerol kinase zeta isoform 6) c4(77407) c5(77408, 77409, 77410, 77411, 77412, 77413) c6( ) c7(bB, bE, T, PX, aw); #c1(DGKZ) c2(NP_001186197) c3(diacylglycerol kinase zeta isoform 7) c4(77414) c5(77415, 77416, 77417, 77418, 77419, 77420) c6( ) c7(bB, bE, T, PX, aw); #c1(DGKZ) c2(NP_003637) c3(diacylglycerol kinase zeta isoform 2) c4(139797) c5(139798, 139799, 139800, 139801, 139802, 139803) c6( ) c7(bB, bE, T, PX, aw); #c1(DGKZ) c2(NP_963290) c3(diacylglycerol kinase zeta isoform 1) c4(262346) c5(262347, 262348, 262349, 262350, 262351, 262352) c6( ) c7(bB, bE, T, PX, aw); #c1(DGKZ) c2(XP_005253239) c3(diacylglycerol kinase zeta isoform X4) c4(272289) c5(272290, 272291, 272292, 272293, 272294, 272295) c6( ) c7(bB, bE, T, PX, aw); #c1(DGKZ) c2(XP_011518723) c3(diacylglycerol kinase zeta isoform X1) c4(313129) c5( ) c6( ) c7(bB, bE, T, PX, aw); #c1(DGKZ) c2(XP_011518725) c3(diacylglycerol kinase zeta isoform X7) c4( ) c5(313130, 313131, 313132, 313133, 313134, 313135) c6( ) c7(bB, bE, T, PX, aw); #c1(DGUOK) c2(NP_550438) c3(deoxyguanosine kinase, mitochondrial isoform a precursor) c4(233494, 233495, 233496) c5(233497, 233498, 233499, 233500, 233501, 233502, 233503, 233504, 233505, 233506, 233507, 233508, 233509, 233510, 233511, 233512, 233513, 233514) c6( ) c7(bL, ck, T, gk, Fr, aE, hb, Ft, al, oj, bt, beV, bZ, aNG, hJ, awL, awl, dF, aoz, kC); #c1(DGUOK) c2(NP_550440) c3(deoxyguanosine kinase, mitochondrial isoform b precursor) c4(233515) c5(233516, 233517, 233518, 233519, 233520, 233521) c6( ) c7(bL, ck, T, gk, Fr, aE, hb, Ft, al, oj, bt, beV, bZ, aNG, hJ, awL, awl, dF, aoz, kC); #c1(DHCR7) c2(NP_001157289) c3(7-dehydrocholesterol reductase) c4(62149, 62150, 62151, 62152, 62153) c5(62154, 62155, 62156, 62157, 62158, 62159, 62160, 62161, 62162, 62163) c6( )c7(bL, dD, aZ, ea, e, al, gN, bB, cL, j, eq, nA, beZ, beW, V, q, o, ad, F, nl, h, hH, ae, A, beX, beY, Az, nh, r, da, QK, dr, bN, bE, vA, el, ZW, B); #c1(DHCR7) c2(XP_011543079) c3(7-dehydrocholesterol reductase isoform X1) c4( )c5(341878, 341879, 341880, 341881, 341882, 341883, 341884, 341885, 341886, 341887, 341888, 341889, 341890, 341891, 341892, 341893, 341894, 341895, 341896, 341897, 341898, 341899, 341900, 341901, 341902, 341903, 341904) c6( )c7(bL, dD, aZ, ea, e, al, gN, bB, cL, j, eq, nA, beZ, beW, V, q, o, ad, F, nl, h, hH, ae, A, beX, beY, Az, nh, r, da, QK, dr, bN, bE, vA, el, ZW, B); #c1(DHDH) c2(NP_055290) c3(trans-1,2-dihydrobenzene-1,2-diol dehydrogenase) c4(180314) c5(180315, 180316, 180317, 180318, 180319, 180320) c6( )c7(al, qV, aA, at, dL, j, ad); #c1(DHFR) c2(NP_000782) c3(dihydrofolate reductase isoform 1) c4(15702) c5(15703, 15704, 15705, 15706, 15707, 15708, 15709, 15710, 15711, 15712) c6( )c7(ng, oO, T, bfb, jv, dA, fR, sQ, fV, iW, jh, cz, bB, bD, j, BF, eg, mF, hF, q, aR, eW, wD, x, av, Na, cq, jZ, oW, aT, dV, h, Nc, dL, KP, lQ, bfc, nJ, dF, Uf, aW, Y, eH, CT, ask, r, eZ, dq, uY, aq, La, ajo, bfa, eF, t, uC, f, ban, bN, ck, mO, y, ac, xK, UP, dB, lp, bE, do, eM, bP, uo, el, wR, ut, oX, k, m, fD, bC, at, ij, CW, us); #c1(DHFRL1) c2(NP_001182572) c3(dihydrofolate reductase, mitochondrial) c4(75073, 75074) c5(75075, 75076, 75077, 75078, 75079, 75080, 75081, 75082, 75083, 75084, 75085, 75086, 75087, 75088, 75089, 75090, 75091, 75092, 75093, 75094) c6( )c7(ip); #c1(DHH) c2(NP_066382) c3(desert hedgehog protein preproprotein) c4(207272) c5(207273, 207274, 207275, 207276, 207277, 207278) c6( ) c7(bfh, UL, aeR, aE, Ew, bfg, bfe, vJ, ael, Kg, aA, bff, aj, UD, hP, fu, bX, aF, bfd, J); #c1(DHODH) c2(NP_001352) c3 (dihydroorotate dehydrogenase (quinone), mitochondrial) c4(1/7230) c5(117231, 117232, 117233, 117234, 117235, 117236) c6( )c7(VJ, jp, qb, Y, fO, dV, aeo, pw, bq, bfi, ai, y); #c1(DHPS) c2(NP_001193903) c3(deoxyhypusine synthase isoform d) c4(83122) c5(83123, 83124, 83125, 83126, 83127, 83128) c6( )c7(TA, T, A, d, jZ, dO, dV, aA, h, aq, aj, aF, pD, fD, B, J, aa); #c1(DHPS) c2(NP_037538) c3(deoxyhypusine synthase isoform b) c4(176098) c5(176099, 176100, 176101, 176102, 176103, 176104) c6( )c7(TA, T, A, d, jZ, dO, dV, aA, h, aq, aj, aF, pD, fD, B, J, aa); #c1(DHPS) c2(XP_011526072) c3(deoxyhypusine synthase isoform X1) c4(320379) c5(320380, 320381, 320382, 320383, 320384, 320385) c6( )c7(TA, T, A, d, jZ, dO, dV, aA, h, aq, aj, aF, pD, fD, B, J, aa); #c1(DHRS11) c2(NP_077284) c3(dehydrogenase/reductase SDR family member 11 precursor) c4(214922, 214923) c5(214924, 214925, 214926, 214927, 214928, 214929, 214930, 214931) c6(214932, 214933) c7(aj, ae, ad); #c1(DHRS11) c2(XP_011523537) c3(dehydrogenase/reductase SDR family member 11 isoform X4) c4( )c5(318054, 318055, 318056, 318057, 318058, 318059, 318060, 318061) c6(318062, 318063) c7(aj, ae, ad); #c1(DHRS2) c2(NP_005785) c3(dehydrogenase/reductase SDR family member 2, mitochondrial isoform 2) c4(159325, 159326) c5(159327, 159328, 159329, 159330, 159331, 159332, 159333, 159334, 159335, 159336, 159337, 159338) c6( )c7(aW, ds, Pg, aT, ahz, K, zo, vi); #c1(DHRS2) c2(NP_878912) c3(dehydrogenase/reductase SDR family member 2, mitochondrial isoform 1) c4(257152) c5(257153, 257154, 257155, 257156, 257157, 257158) c6( )c7(aW, ds, Pg, aT, ahz, K, zo, vi); #c1(DHRS4) c2(NP_001269916) c3(dehydrogenase/reductase SDR family member 4 isoform 2) c4(103183, 103184) c5(103185, 103186, 103187, 103188, 103189, 103190, 103191, 103192, 103193, 103194, 103195, 103196) c6( )c7(ff, en); #c1(DHRS4) c2(NP_001269917) c3(dehydrogenase/reductase SDR family member 4 isoform 3) c4(103197, 103198, 103199) c5(103200, 103201, 103202, 103203, 103204, 103205, 103206, 103207, 103208, 103209, 103210, 103211) c6( )c7(ff, en); #c1(DHRS4) c2(NP_001269918) c3(dehydrogenase/reductase SDR family member 4 isoform 4) c4(103212) c5(103213, 103214, 103215, 103216, 103217, 103218) c6( )c7(ff, en); #c1(DHRS4) c2(NP_001269919) c3(dehydrogenase/reductase SDR family member 4 isoform 5) c4(103219, 103220) c5(103221, 103222, 103223, 103224, 103225, 103226, 103227, 103228, 103229, 103230, 103231, 103232) c6( )c7(ff, en); #c1(DHRS4) c2(NP_001269920) c3(dehydrogenase/reductase SDR family member 4 isoform 6) c4(103233, 103234) c5(103235, 103236, 103237, 103238, 103239, 103240, 103241, 103242, 103243, 103244, 103245, 103246) c6( )c7(ff, en); #c1(DHRS4) c2(NP_066284) c3(dehydrogenase/reductase SDR family member 4 isoform 1) c4(206982, 206983) c5(206984, 206985, 206986, 206987, 206988, 206989) c6( )c7(ff, en); #c1(DHRS7C) c2(NP_001099041) c3(dehydrogenase/reductase SDR family member 7C isoform 2 precursor) c4(41778) c5(41779, 41780, 4/781, 41782, 41783, 41784, 41785, 41786) c6(41787, 41788) c7(cd); #c1(DHRS7C) c2(NP_001207422) c3(dehydrogenase/reductase SDR family member 7C isoform 1 precursor) c4(83700) c5(83701, 83702, 83703, 83704, 83705, 83706, 83707, 83708) c6(83709, 83710) c7(cd); #c1(DHRS9) c2(NP_001276692) c3(dehydrogenase/reductase SDR family member 9 isoform 2) c4(108275, 108276, 108277) c5(108278, 108279, 108280, 108281, 108282, 108283, 108284, 108285, 108286, 108287, 108288, 108289) c6( )c7(pu, fR, aW, ha, aT, ci, dw, ae, ad); #c1(DHRS9) c2(NP_954674) c3(dehydrogenase/reductase SDR family member 9 isoform 1 precursor) c4(261251) c5(261252, 261253, 261254, 261255, 261256, 261257, 261258, 261259, 261260, 261261, 261262, 261263, 261264, 261265, 261266, 261267, 261268, 261269, 261270, 261271, 261272, 261273, 261274) c6( )c7(pu, fR, aW, ha, aT, ci, dw, ae, ad); #c1(DHRS9) c2(XP_011508778) c3(dehydrogenase/reductase SDR family member 9 isoform X2) c4( )c5(299371, 299372, 299373, 299374, 299375) c6( )c7(pu, fR, aW, ha, aT, ci, dw, ae, ad); #c1(DHTKD1) c2(NP_061176) c3(probable 2-oxoglutarate dehydrogenase E1 component DHKTD1, mitochondrial) c4(199761, 199762, 199763, 199764) c5(199765, 199766, 199767, 199768, 199769, 199770, 199771, 199772, 199773, 199774, 199775, 199776, 199777, 199778, 199779, 199780, 199781, 199782, 199783, 199784, 199785, 199786, 199787, 199788) c6( )c7(bT, bfj, bfk); #c1(DHX15) c2(NP_001349) c3(putative pre-mRNA-splicing factor ATP-dependent RNA helicase DHX15) c4(1/7209, 117210) c5(117211, 117212, 117213, 117214, 117215, 117216, 117217, 117218, 117219, 117220, 117221, 117222) c6( )c7(Bo, aA, aF, aYe, aDJ, hX); #c1(DHX16) c2(NP_001157711) c3(putative pre-mRNA-splicing factor ATP-dependent RNA helicase DHX16 isoform 2) c4(62428) c5(62429, 62430, 62431, 62432, 62433, 62434, 62435, 62436, 62437, 62438, 62439, 62440, 62441, 62442, 62443, 62444, 62445, 62446, 62447, 62448, 62449, 62450, 62451, 62452, 62453, 62454, 62455) c6( )c7(bL, T, eR, jx, bej, aVK, V, q, eA, bel, ahD, MR, bW, dV, aT, K, lQ, bV, jf, A, lR, aW, Y, eZ, r, lo, nC, pb, dS, c, Jr, U, uo, aYe, y, bek); #c1(DHX16) c2(NP_003578) c3(putative pre-mRNA-splicing factor ATP-dependent RNA helicase DHX16 isoform 1) c4(139323, 139324, 139325, 139326) c5(139327, 139328, 139329, 139330, 139331, 139332, 139333, 139334, 139335, 139336, 139337, 139338, 139339, 139340, 139341, 139342, 139343, 139344, 139345, 139346, 139347, 139348, 139349, 139350, 139351, 139352, 139353, 139354, 139355, 139356, 139357, 139358, 139359, 139360, 139361, 139362, 139363, 139364, 139365) c6( )c7(bL, T, eR, jx, bej, aVK, V, q, eA, bel, ahD, MR, bW, dV, aT, K, lQ, bV, jf, A, lR, aW, Y, eZ, r, lo, nC, pb, dS, c, Jr, U, uo, aYe, y, bek); #c1(DHX16) c2(XP_011513242) c3(putative pre-mRNA-splicing factor ATP-dependent RNA helicase DHX16 isoform X2) c4(305472, 305473) c5(305474, 305475, 305476, 305477, 305478, 305479, 305480, 305481, 305482, 305483, 305484, 305485, 305486, 305487, 305488, 305489, 305490, 305491, 305492, 305493, 305494, 305495, 305496, 305497, 305498, 305499, 305500) c6( )c7(bL, T, eR, jx, bej, aVK, V, q, eA, bel, ahD, MR, bW, dV, aT, K, lQ, bV, jf, A, lR, aW, Y, eZ, r, lo, nC, pb, dS, c, Jr, U, uo, aYe, y, bek); #c1(DHX16) c2(XP_011546614) c3(putative pre-mRNA-splicing factor ATP-dependent RNA helicase DHX16 isoform X2) c4(345435) c5(345436, 345437, 345438, 345439, 345440, 345441) c6( )c7(bL, T, eR, jx, bej, aVK, V, q, eA, bel, ahD, MR, bW, dV, aT, K, lQ, bV, jf, A, lR, aW, Y, eZ, r, lo, nC, pb, dS, c, Jr, U, uo, aYe, y, bek); #c1(DHX32) c2(NP_060650) c3 (putative pre-mRNA-splicing factor ATP-dependent RNA helicase DHX32) c4(196980, 196981, 196982, 196983) c5(196984, 196985, 196986, 196987, 196988, 196989, 196990, 196991, 196992, 196993, 196994, 196995, 196996, 196997, 196998, 196999, 197000, 197001, 197002, 197003, 197004, 197005, 197006, 197007) c6( )c7(ck, x, T, jh, r, j, k, q); #c1(DHX34) c2(NP_055496) c3(probable ATP-dependent RNA helicase DHX34) c4(181541) c5(181542, 181543, 181544, 181545, 181546, 181547) c6( )c7(aDJ, bM, j, ac); #c1(DHX34) c2(XP_011525854) c3(probable ATP-dependent RNA helicase DHX34 isoform X2) c4(320280) c5(320281, 320282, 320283, 320284, 320285, 320286) c6( )c7(aDJ, bM, j, ac); #c1(DHX36) c2(NP_001107869) c3(ATP-dependent RNA helicase DHX36 isoform 2) c4(43078, 43079) c5(43080, 43081, 43082, 43083, 43084, 43085, 43086, 43087, 43088, 43089, 43090, 43091) c6( )c7(aW, q, r, aT); #c1(DHX36) c2(NP_065916) c3(ATP-dependent RNA helicase DHX36 isoform 1) c4(206229, 206230) c5(206231, 206232, 206233, 206234, 206235, 206236, 206237, 206238, 206239, 206240, 206241, 206242) c6( )c7(aW, q, r, aT); #c1(DHX38) c2(NP_054722) c3(pre-mRNA-splicing factor ATP-dependent RNA helicase PRP16) c4(177359, 177360) c5(177361, 177362, 177363, 177364, 177365, 177366) c6( )c7(nC, bfl); #c1(DHX38) c2(XP_005256326) c3(pre-mRNA-splicing factor ATP-dependent RNA helicase PRP16 isoform X2) c4( )c5(274114, 274115, 274116, 274117, 274118, 274119) c6( )c7(nC, bfl); #c1(DHX40) c2(NP_001159773) c3(probable ATP-dependent RNA helicase DHX40 isoform 2) c4(64785) c5(64786, 64787, 64788, 64789, 64790, 64791) c6( )c7(KT, T, bf, bE, e, M, bB, cy, aj, dQ, y, V, J); #c1(DHX40) c2(NP_078888) c3(probable ATP-dependent RNA helicase DHX40 isoform 1) c4(215933, 215934, 215935) c5(215936, 215937, 215938, 215939, 215940, 215941, 215942, 215943, 215944, 215945, 215946, 215947, 215948, 215949, 215950, 215951, 215952, 215953) c6( )c7(kT, T, bf, bE, e, M, bB, cy, aj, dl, y, V, J); #c1(DHX58) c2(NP_077024) c3(probable ATP-dependent RNA helicase DHX58) c4(214789, 214790, 214791, 214792) c5(214793, 214794, 214795, 214796, 214797, 214798, 214799, 214800, 214801, 214802, 214803, 214804) c6( )c7(ds, lo, V, aFc, vi); #c1(DHX58) c2(XP_011523526) c3(probable ATP-dependent RNA helicase DHX58 isoform X1) c4( )c5(318033, 318034, 318035, 318036, 318037, 318038, 318039, 318040, 318041, 318042, 318043, 318044) c6( )c7(ds, lo, V, aFc, vi); #c1(DHX8) c2(NP_001289552) c3(ATP-dependent RNA helicase DHX8 isoform 2) c4(114267) c5(114268, 114269, 114270, 114271, 114272, 114273) c6( )c7(aDJ, bM, j, ac); #c1(DHX8) c2(NP_004932) c3(ATP-dependent RNA helicase DHX8 isoform 1) c4(151061) c5(151062, 151063, 151064, 151065, 151066, 151067) c6( )c7(aDJ, bM, j, ac); #c1(DHX9) c2(NP_001348) c3(ATP-dependent RNA helicase A) c4(117195, 117196) c5(117197, 117198, 117199, 117200, 117201, 117202, 117203, 117204, 117205, 117206, 117207, 117208) c6( )c7(Bv, wr, aW, ds, AH, eZ, aT, cm, jh, lo, fL, jj, bfm, kS, mC); #c1(DIABLO) c2(NP_001265233) c3(diablo homolog, mitochondrial isoform 2) c4(98014) c5(98015, 98016, 98017, 98018, 98019, 98020, 98021, 98022, 98023, 98024, 98025) c6( )c7(T, wu, if, ht, eX, fd, bB, n, fr, ci, qN, eA, ad, ft, Yc, Bc, aT, vH, eV, bfn, K, hu, wm, eu, B, gP, xR, aW, Y, ff, eH, aq, en, g, eF, t, cm, bz, ik, dr, rR, eM, bE, wz, m, bt, it, X, cn); #c1(DIABLO) c2(NP_001265271) c3(diablo homolog, mitochondrial isoform 3) c4(98094) c5(98095, 98096, 98097, 98098, 98099, 98100, 98101, 98102, 98103, 98104, 98105) c6( )c7(T, wu, if, ht, eX, fd, bB, n, fr, ci, qN, eA, ad, ft, Yc, Bc, aT, vH, eV, bfn, K, hu, wm, eu, B, gP, xR, aW, Y, ff, eH, aq, en, g, eF, t, cm, bz, ik, dr, rR, eM, bE, wz, m, bt, it, X, cn); #c1(DIABLO) c2(NP_063940) c3(diablo homolog, mitochondrial isoform 1 precursor) c4(202255) c5(202256, 202257, 202258, 202259, 202260, 202261, 202262, 202263, 202264, 202265, 202266, 202267, 202268, 202269, 202270, 202271, 202272, 202273, 202274, 202275, 202276, 202277, 202278, 202279, 202280, 202281, 202282, 202283, 202284, 202285, 202286, 202287, 202288, 202289, 202290, 202291, 202292, 202293, 202294, 202295, 202296, 202297, 202298, 202299, 202300, 202301, 202302, 202303, 202304, 202305, 202306, 202307, 202308, 202309, 202310, 202311, 202312, 202313, 202314, 202315, 202316, 202317, 202318, 202319, 202320, 202321, 202322, 202323, 202324, 202325, 202326, 202327, 202328, 202329, 202330, 202331, 202332, 202333, 202334, 202335, 202336, 202337, 202338, 202339, 202340, 202341, 202342, 202343, 202344, 202345, 202346, 202347, 202348, 202349, 202350) c6(202351) c7(T, wu, if, ht, eX, fd, bB, n, fr, ci, qN, eA, ad, ft, Yc, Bc, aT, vH, eV, bfn, K, hu, wm, eu, B, gP, xR, aW, Y, ff, eH, aq, en, g, eF, t, cm, bz, ik, dr, rR, eM, bE, wz, m, bt, it, X, cn); #c1(DIAPL0) c2(NP_001073280) c3(protein diaphanous homolog 1 isoform 2) c4(35720, 35721, 35722) c5(35723, 35724, 35725, 35726, 35727, 35728, 35729, 35730, 35731, 35732, 35733, 35734, 35735, 35736, 35737, 35738, 35739, 35740) c6( )c7(ck, T, dA, bE, Y, bfo, axv, aDH, r, bB, fb, Bh, q); #c1(DIAPL0) c2(NP_005210) c3 (protein diaphanous homolog 1 isoform 1) c4(154028, 154029, 154030) c5(154031, 154032, 154033, 154034, 154035, 154036, 154037, 154038, 154039, 154040, 154041, 154042, 154043, 154044, 154045, 154046, 154047, 154048, 154049, 154050, 154051, 154052, 154053) c6(154054, 154055) c7(ck, T, dA, bE, Y, bfo, axv, aOH, r, bB, fb, Bh, q); #c1(DIAPL0) c2(XP_011535875) c3(protein diaphanous homolog 1 isoform X2) c4(333144) c5( ) c6( )c7(ck, T, dA, bE, Y, bfo, axv, aDH, r, bB, fb, Bh, q); #c1(DIAPH2) c2(NP_006720) c3(protein diaphanous homolog 2 isoform 156) c4(167399, 167400) c5(167401, 167402, 167403, 167404, 167405, 167406, 167407, 167408, 167409, 167410, 167411, 167412, 167413, 167414, 167415, 167416, 167417, 167418, 167419, 167420, 167421, 167422, 167423, 167424, 167425, 167426, 167427, 167428, 167429, 167430, 167431, 167432, 167433, 167434, 167435, 167436) c6( )c7(W, dB, il, pQ, aeS, zZ, bfp, Lg); #c1(DIAPH2) c2(NP_009293) c3(protein diaphanous homolog 2 isoform (2C) c4(171795, 171796) c5( )c6( )c7(W, dB, il, pQ, aeS, zZ, bfp, Lg); #c1(DIAPH3) c2(NP_001035982) c3(protein diaphanous homolog 3 isoform a) c4(33610, 33611) c5(33612, 33613, 33614, 33615, 33616, 33617, 33618, 33619, 33620, 33621, 33622, 33623) c6( )c7(af, By, bfq, m, t, cd, bN); #c1(DIAPH3) c2(NP_001245295) c3(protein diaphanous homolog 3 isoform c) c4(92991) c5(92992, 92993, 92994, 92995, 92996, 92997) c6( )c7(af, By, bfq, m, t, cd, bN); #c1(DIAPH3) c2(NP_001245296) c3 (protein diaphanous homolog 3 isoform d) c4(92998) c5(92999, 93000, 93001, 93002, 93003, 93004) c6( )c7(af, By, bfq, m, t, cd, bN); #c1(DIAPH3) c2(NP_001245297) c3(protein diaphanous homolog 3 isoform e) c4(93005) c5(93006, 93007, 93008, 93009, 93010, 93011) c6( )c7(af, By, bfq, m, t, cd, bN); #c1(DIAPH3) c2(NP_001245298) c3(protein diaphanous homolog 3 isoform f) c4(93012, 93013) c5(93014, 93015, 93016, 93017, 93018, 93019, 93020, 93021, 93022, 93023, 93024, 93025, 93026, 93027, 93028, 93029, 93030, 93031) c6( )c7(af, By, bfq, m, t, cd, bN); #c1(DIAPH3) c2(XP_011533560) c3(protein diaphanous homolog 3 isoform X1) c4(329675) c5( ) c6( )c7(af, By, bfq, m, t, cd, bN); #c1(DIAPH3) c2(XP_011533563) c3(protein diaphanous homolog 3 isoform X4) c4(329676, 329677) c5( )c6( )c7(af, By, bfq, m, t, cd, bN); #c1(DICER1) c2(NP_001182502) c3(endoribonuclease Dicer isoform 2) c4(74983) c5(74984, 74985, 74986, 74987, 74988, 74989, 74990, 74991) c6( )c7(T, bB, amj, ci, bfr, Ou, K, eL, aW, Ku, g, cm, bw, Uk, bfw, eM, nB, cj, m, fW, iB, RX, bfx, n, fr, bfv, nA, aOr, br, jz, Yc, eV, dL, ey, adz, bfy, W, bP, hk, bft, hL, B, bL, eX, aA, hy, v, yp, q, er, eA, anj, aT, bA, jf, iF, Lu, ND, vT, Or, Y, bfz, r, hf, cl, t, f, iK, ck, bt, bs, bfs, KO, anu, ad, hX, dP, hd, alq, hD, yF, BN, bfu, aF, bz, es, EW, bE, rH, ne); #c1(DICER1) c2(NP_001278557) c3(endoribonuclease Dicer isoform 1) c4(109821, 109822) c5(109823, 109824, 109825, 109826, 109827, 109828, 109829, 109830, 109831, 109832, 109833, 109834, 109835, 109836, 109837, 109838, 109839, 109840, 109841, 109842, 109843, 109844, 109845, 109846, 109847, 109848, 109849, 109850, 109851, 109852, 109853, 109854) c6( )c7(T, bB, amj, ci, bfr, Ou, K, eL, aW, Ku, g, cm, bw, Uk, bfw, eM, nB, cj, m, fW, iB, RX, bfx, n, fr, bfv, nA, aOr, br, jz, Yc, eV, dl, ey, adz, bfy, W, bP, hk, bft, hL, B, bL, eX, aA, hy, v, yp, q, er, eA, anj, aT, bA, jf, iF, Lu, ND, vT, Qr, Y, bfz, r, hf, cl, t, f, ik, ck, bt, bs, bfs, KO, anu, ad, hX, dP, hd, aOq, hD, yF, BN, bfu, aF, bz, es, EW, bE, rH, ne); #c1(DICER1) c2(XP_011534907) c3(endoribonuclease Dicer isoform X3) c4(331427) c5( )c6( )c7(T, bB, amj, ci, bfr, Ou, K, eL, aW, Ku, g, cm, bw, Uk, bfw, eM, nB, cj, m, fW, iB, RX, bfx, n, fr, bfv, nA, aOr, br, jz, Yc, eV, dL, ey, adz, bfy, W, bP, hk, bft, hL, B, bL, eX, aA, hy, v, yp, q, er, eA, anj, aT, bA, jf, iF, Lu, ND, vT, Or, Y, bfz, r, hf, cl, t, f, ik, ck, bt, bs, bfs, KO, anu, ad, hX, dP, hd, aOg, hD, yF, BN, bfu, af, bz, es, EW, bE, rH, ne); #c1(DID01) c2(NP_071388) c3(death-inducer obliterator 1 isoform a) c4(211333) c5(211334, 211335, 211336, 211337, 211338, 211339, 211340, 211341, 211342, 211343, 211344, 211345, 211346, 211347, 211348, 211349, 211350, 211351, 211352, 211353, 211354, 211355, 211356, 211357, 211358) c6( )c7(es, T, hd, py, ac, iA, Y, hD, uu, bw, af); #c1(DID01) c2(NP_149072) c3(death-inducer obliterator 1 isoform c) c4(226983, 226984) c5(226985, 226986, 226987, 226988, 226989, 226990, 226991, 226992, 226993, 226994) c6( )c7(es, T, hd, py, ac, iA, Y, HD, uu, bw, af); #c1(DID01) c2(NP_542987) c3(death-inducer obliterator 1 isoform b) c4(233132, 233133) c5(233134, 233135, 233136, 233137, 233138, 233139, 233140, 233141, 233142, 233143) c6( )c7(es, T, hd, py, ac, iA, Y, hD, uu, bw, af); #c1(DIEXF) c2(NP_055203) c3(digestive organ expansion factor homolog) c4(179774, 179775) c5(179776, 179777, 179778, 179779, 179780, 179781) c6( )c7(se, sq); #c1(DIEXF) c2(XP_011507720) c3(digestive organ expansion factor homolog isoform X2) c4( )c5(297802, 297803, 297804, 297805, 297806, 297807) c6( )c7(se, sq); #c1(DIO2) c2(NP_000784) c3(type II iodothyronine deiodinase isoform a) c4(15713, 15714, 15715) c5(15716, 15717, 15718, 15719, 15720, 15721, 15722, 15723, 15724, 15725, 15726, 15727, 15728, 15729, 15730, 15731) c6( )c7(bL, oq, cz, hz, aj, v, ci, nA, ml, J, dP, Vp, adf, hd, ib, cc, hD, bK, nJ, NM, af, VX, GR, qJ, nM, nB, bfA, sG, hR, ei, gT, AN); #c1(DIO2) c2(NP_001007024) c3(type Il iodothyronine deiodinase isoform b) c4(22381, 22382) c5(22383, 22384, 22385, 22386, 22387, 22388) c6( )c7(bL, oq, cz, hz, aj, v, ci, nA, ml, J, dP, Vp, adf, hd, ib, cc, hD, bK, nJ, NM, af, VX, GR, qJ, nM, nB, bfA, sG, hR, ei, gT, AN); #c1(DIO2) c2(NP_001229431) c3(type Il iodothyronine deiodinase isoform c) c4( )c5(84410, 84411, 84412, 84413, 84414) c6( )c7(bL, oq, cz, hz, aj, v, ci, nA, ml, J, dP, Vp, adf, hd, ib, cc, hD, bK, nJ, NM, af, VX, GR, qJ, nM, nB, bfA, sG, hR, ei, gT, AN); #c1(DIO2) c2(NP_001229432) c3 (type II iodothyronine deiodinase isoform d) c4( )c5(84415, 84416, 84417, 84418, 84419, 84420) c6( )c7(bL, oq, cz, hz, aj, v, ci, nA, ml, J, dP, Vp, adf, hd, ib, cc, hD, bK, nJ, NM, af, VX, GR, qJ, nM, nB, bfA, sG, hR, ei, gT, AN); #c1(DIO3) c2(NP_001353) c3(type III iodothyronine deiodinase) c4(1/7237, 117238) c5(117239, 117240, 117241, 117242, 117243, 117244, 117245, 117246, 117247, 117248, 117249, 117250) c6( )c7(T, A, B, VX, nM, aw, aq, jj, iR, gT, cd); #c1(DIP2A) c2(NP_001139587) c3(disco-interacting protein 2 homolog A isoform f) c4(59539, 59540) c5(59541, 59542, 59543, 59544, 59545, 59546, 59547, 59548, 59549, 59550, 59551) c6( )c7(vr, ac); #c1(DIP2A) c2(NP_001139588) c3(disco-interacting protein 2 homolog A isoform g) c4(59552) c5(59553, 59554, 59555, 59556, 59557, 59558) c6( )c7(vr, ac); #c1(DIP2A) c2(NP_055966) c3(disco-interacting protein 2 homolog A isoform a) c4(184760) c5(184761, 184762, 184763, 184764, 184765, 184766, 184767, 184768, 184769, 184770, 184771, 184772, 184773, 184774, 184775, 184776, 184777, 184778, 184779) c6( )c7(vr, ac); #c1(DIP2A) c2(NP_996772) c3(disco-interacting protein 2 homolog A isoform b) c4(264508) c5(264509, 264510, 264511, 264512, 264513, 264514) c6( )c7(vr, ac); #c1(DIP2A) c2(NP_996773) c3(disco-interacting protein 2 homolog A isoform c) c4(264515) c5( )c6( ) c7(vr, ac); #c1(DIP2A) c2(XP_011527794) c3(disco-interacting protein 2 homolog A isoform X3) c4(322762) c5( )c6( )c7(vr, ac); #c1(DIP2A) c2(XP_011527803) c3(disco-interacting protein 2 homolog A isoform X12) c4(322763) c5(322764, 322765, 322766, 322767, 322768, 322769) c6( )c7(vr, ac); #c1(DIP2B) c2(NP_775873) c3(disco-interacting protein 2 homolog B) c4(250535) c5(250536, 250537, 250538, 250539, 250540, 250541) c6( )c7(bfB, nA, aan); #c1(DIP2B) c2(XP_006719583) c3(disco-interacting protein 2 homolog B isoform X2) c4(292654) c5(292655, 292656, 292657, 292658, 292659, 292660) c6( )c7(bfB, nA, aan); #c1(DIP2C) c2(NP_055789) c3(disco-interacting protein 2 homolog C) c4(183513, 183514) c5(183515, 183516, 183517, 183518, 183519, 183520, 183521, 183522, 183523, 183524, 183525, 183526, 183527, 183528, 183529, 183530, 183531, 183532) c6( )c7(aw, ft, af, ac); #c1(DIP2C) c2(XP_005252483) c3(disco-interacting protein 2 homolog C isoform X10) c4(271813, 271814, 271815) c5(271816, 271817, 271818, 271819, 271820, 271821) c6( )c7(aw, ft, af, ac); #c1(DIP2C) c2(XP_005252484) c3(disco-interacting protein 2 homolog C isoform X4) c4(271822) c5( )c6( )c7(aw, ft, af, ac); #c1(DIP2C) c2(XP_005252485) c3(disco-interacting protein 2 homolog C isoform X6) c4(271823) c5( )c6( )c7(aw, ft, af, ac); #c1(DIP2C) c2(XP_005252487) c3(disco-interacting protein 2 homolog C isoform X8) c4(271824) c5(271825) c6( )c7(aw, ft, af, ac); #c1(DIP2C) c2(XP_011517734) c3(disco-interacting protein 2 homolog C isoform X7) c4( ) c5(311528, 311529, 311530, 311531, 311532, 311533) c6( )c7(aw, ft, af, ac); #c1(DIP2C) c2(XP_011517735) c3(disco-interacting protein 2 homolog C isoform X9) c4( )c5(311534, 311535, 311536, 311537, 311538, 311539, 311540, 311541, 311542, 311543, 311544, 311545, 311546, 311547, 311548, 311549, 311550) c6( )c7(aw, ft, af, ac); #c1(DIRAS1) c2(NP_660156) c3(GTP-binding protein Di-Ras1) c4( )c5(240417, 240418, 240419, 240420, 240421) c6( )c7(d, apn, h, aiT, as, it, eA); #c1(DIRAS1) c2(XP_011526015) c3(GTP-binding protein Di-Ras1 isoform X1) c4(320367) c5(320368) c6( )c7(d, apn, h, aiT, as, it, eA); #c1(DIRAS2) c2(NP_060064) c3(GTP-binding protein Di-Ras2) c4(193976) c5(193977, 193978, 193979, 193980, 193981, 193982) c6( )c7(j); #c1(DIRC1) c2(XP_011508847) c3(disrupted in renal carcinoma protein 1 isoform X1) c4(299430) c5(299431, 299432, 299433, 299434, 299435, 299436) c6( )c7(es, T); #c1(DIRC2) c2(NP_116228) c3(disrupted in renal carcinoma protein 2) c4(225858) c5(225859, 225860, 225861, 225862, 225863, 225864) c6( ) c7(es, fr, ci, iA); #c1(DIS3) c2(NP_001121698) c3(exosome complex exonuclease RRP44 isoform b) c4(46097) c5(46098, 46099, 46100, 46101, 46102, 46103) c6( )c7(T, s, r, fd, gl, ci, q, er); #c1(DIS3) c2(NP_055768) c3(exosome complex exonuclease RRP44 isoform a) c4(183372) c5(183373, 183374, 183375, 183376, 183377, 183378, 183379, 183380, 183381, 183382, 183383, 183384) c6( )c7(T, s, r, fd, gl, ci, q, er); #c1(DIS3) c2(XP_005266351) c3(exosome complex exonuclease RRP44 isoform X2) c4(279964) c5( )c6( )c7(T, s, r, fd, gl, ci, q, er); #c1(DIS3L) c2(NP_001137160) c3(DIS3-like exonuclease 1 isoform 1) c4(55067) c5(55068, 55069, 55070, 55071, 55072, 55073, 55074, 55075) c6( )c7(W); #c1(DIS3L) c2(NP_588616) c3(DIS3-like exonuclease 1 isoform 2) c4(234832, 234833, 234834, 234835, 234836, 234837) c5(234838, 234839, 234840, 234841, 234842) c6( )c7(W); #c1(DIS3L) c2(XP_005254203) c3(DIS3-like exonuclease 1 isoform X3) c4( )c5(273070, 273071, 273072, 273073, 273074, 273075, 273076, 273077, 273078, 273079, 273080, 273081, 273082, 273083, 273084, 273085, 273086, 273087, 273088, 273089, 273090, 273091, 273092, 273093, 273094, 273095, 273096, 273097, 273098) c6( )c7(W); #c1(DIS3L2) c2(NP_001244210) c3(DIS3-like exonuclease 2 isoform 2) c4(92033) c5(92034, 92035, 92036, 92037, 92038, 92039) c6( )c7(T, er); #c1(DIS3L2) c2(NP_001244211) c3(DIS3-like exonuclease 2 isoform 3) c4(92040, 92041) c5(92042, 92043, 92044, 92045, 92046, 92047, 92048, 92049, 92050, 92051, 92052, 92053, 92054, 92055, 92056, 92057, 92058, 92059) c6( )c7(T, er); #c1(DIS3L2) c2(NP_689596) c3(DIS3-like exonuclease 2 isoform 1) c4(243135, 243136, 243137, 243138) c5(243139, 243140, 243141, 243142, 243143, 243144, 243145, 243146, 243147, 243148, 243149, 243150, 243151, 243152, 243153, 243154, 243155, 243156, 243157, 243158, 243159, 243160) c6( )c7(T, er); #c1(DISC1) c2(NP_001012975) c3(disrupted in schizophrenia 1 protein isoform Lv) c4(25361, 25362, 25363) c5(25364, 25365, 25366, 25367, 25368, 25369) c6( )c7(bfF, iH, bfC, aw, bO, ti, bS, ct, nA, wy, bfD, aR, gF, ot, hb, Fo, bdp, bK, K, ahg, dQ, he, af, sj, kS, bfE, gl, Z, cm, jb, bN, c, cA, aND, cj, vl, sv, Rm, uJ, xg); #c1 (DISC1) c2(NP_001012976) c3(disrupted in schizophrenia 1 protein isoform Es) c4(25370) c5(25371, 25372, 25373, 25374, 25375, 25376) c6( ) c7(bfF, iH, bfC, aw, bO, ti, bS, ct, nA, wy, bfD, aR, gF, ot, hb, Fo, bdp, bK, K, ahg, dQ, he, af, sj, kS, bfE, gl, Z, cm, jb, bN, c, cA, aND, cj, vl, sv, Rm, uJ, xg); #c1(DISC1) c2(NP_001012977) c3(disrupted in schizophrenia 1 protein isoform S) c4(25377) c5(25378, 25379, 25380, 25381, 25382, 25383) c6( )c7(bfF, iH, bfC, aw, bO, ti, bS, ct, nA, wy, bfD, aR, gF, ot, hb, Fo, bdp, bK, K, ahg, dQ, he, af, sj, kS, bfE, gl, Z, cm, jb, bN, c, cA, aND, cj, vL, sv, Rm, uJ, xg); #c1(DISC1) c2(NP_001158009) c3(disrupted in schizophrenia 1 protein isoform a) c4(62997, 62998) c5(62999, 63000, 63001, 63002, 63003, 63004) c6( )c7(bfF, iH, bfC, aw, bO, ti, bS, ct, nA, wy, bfD, aR, gF, ot, hb, Fo, bdp, bK, K, ahg, dQ, he, af, sj, kS, bfE, gl, Z, cm, jb, bN, c, cA, aND, cj, vL, sv, Rm, uJ, xg); #c1(DISC1) c2(NP_001158010) c3(disrupted in schizophrenia 1 protein isoform b) c4(63005) c5(63006, 63007, 63008, 63009, 63010, 63011) c6( )c7(bfF, iH, bfC, aw, bO, ti, bS, ct, nA, wy, bfD, aR, gF, ot, hb, Fo, bdp, bK, K, ahg, dQ, he, af, sj, KS, bfE, gl, Z, cm, jb, bN, c, cA, aND, cj, vL, sv, Rm, uJ, xg); #c1(DISC1) c2(NP_001158011) c3(disrupted in schizophrenia 1 protein isoform c) c4(63012) c5(63013, 63014, 63015, 63016, 63017, 63018, 63019, 63020, 63021) c6( ) c7(bfF, iH, bfC, aw, bO, ti, bS, ct, nA, wy, bfD, aR, gF, ot, hb, Fo, bdp, bK, K, ahg, dQ, he, af, sj, KS, bfE, gl, Z, cm, jb, bN, c, cA, aND, cj, vL, sv, Rm, uJ, xg); #c1(DISC1) c2(NP_001158012) c3(disrupted in schizophrenia 1 protein isoform d) c4(63022) c5(63023, 63024, 63025, 63026, 63027, 63028, 63029, 63030, 63031, 63032, 63033, 63034) c6( )c7(bfF, iH, bfC, aw, bO, ti, bS, ct, nA, wy, bfD, aR, gF, ot, hb, Fo, bdp, bK, K, ahg, dQ, he, af, sj, kS, bfE, gl, Z, cm, jb, bN, c, cA, aND, cj, vL, sv, Rm, uJ, xg); #c1(DISC1) c2(NP_001158013) c3(disrupted in schizophrenia 1 protein isoform e) c4(63035) c5(63036, 63037, 63038, 63039, 63040, 63041) c6( )c7(bfF, iH, bfC, aw, bO, ti, bS, ct, nA, wy, bfD, aR, gF, ot, hb, Fo, bdp, bK, K, ahg, dQ, he, af, sj, kS, bfE, gl, Z, cm, jb, bN, c, cA, aND, cj, vL, sv, Rm, uJ, xg); #c1(DISC1) c2(NP_001158017) c3(disrupted in schizophrenia 1 protein isoform h) c4(63042) c5(63043, 63044, 63045, 63046, 63047, 63048, 63049) c6( )c7(bfF, iH, bfC, aw, bO, ti, bS, ct, nA, wy, bfD, aR, gF, ot, hb, Fo, bdp, bK, K, ahg, dQ, he, af, sj, kS, bfE, gl, Z, cm, jb, b, c, cA, aND, cj, vL, sv, Rm, uJ, xg); #c1(DISC1) c2(NP_001158019) c3(disrupted in schizophrenia 1 protein isoform i) c4(63050) c5(63051, 63052, 63053, 63054, 63055, 63056) c6( )c7(bfF, iH, bfC, aw, bO, ti, bS, ct, nA, wy, bfD, aR, gF, ot, hb, Fo, bdp, bK, K, ahg, dl, he, af, sj, kS, bfE, gl, Z, cm, jb, bN, c, cA, aND, cj, vL, sv, Rm, uJ, xg); #c1(DISC1) c2(NP_001158020) c3(disrupted in schizophrenia I protein isoform k) c4( )c5(63057, 63058, 63059, 63060, 63061, 63062) c6( )c7(bfF, iH, bfC, aw, bO, ti, bS, ct, nA, wy, bfD, aR, gF, ot, hb, Fo, bdp, bK, K, ahg, dQ, he, af, sj, kS, bfE, gl, Z, cm, jb, bN, c, cA, aND, cj, vL, sv, Rm, uJ, xg); #c1(DISC1) c2(NP_00158021) c3(disrupted in schizophrenia 1 protein isoform 1) c4( )c5(63063, 63064, 63065, 63066, 63067) c6( )c7(bfF, iH, bfC, aw, bO, ti, bS, ct, nA, wy, bfD, aR, gF, ot, hb, Fo, bdp, bK, K, ahg, dQ, he, af, sj, kS, bfE, gl, Z, cm, jb, bN, c, cA, aND, cj, vL, sv, Rm, uJ, xg); #c1(DISC1) c2(NP_001158025) c3(disrupted in schizophrenia 1 protein isoform p) c4(63068) c5(63069, 63070, 63071, 63072, 63073, 63074) c6( )c7(bfF, iH, bfC, aw, bO, ti, bS, ct, nA, wy, bfD, aR, gF, ot, hb, Fo, bdp, bK, K, ahg, dQ, he, af, sj, KS, bfE, gl, Z, cm, jb, bN, c, cA, aND, cj, vL, sv, Rm, uJ, xg); #c1(DISC1) c2(NP_061132) c3(disrupted in schizophrenia 1 protein isoform L) c4(199428) c5(199429, 199430, 199431, 199432, 199433, 199434) c6( ) c7(bfF, iH, bfC, aw, bO, ti, bS, ct, nA, wy, bfD, aR, gF, ot, hb, Fo, bdp, bK, K, ahg, dQ, he, af, sj, KS, bfE, gl, Z, cm, jb, bN, c, cA, aND, cj, vl, sv, Rm, uJ, xg); #c1(DISP1) c2(NP_116279) c3(protein dispatched homolog 1) c4(226066, 226067) c5(226068, 226069, 226070, 226071, 226072, 226073, 226074, 226075, 226076, 226077, 226078, 226079) c6( )c7(ZW, eM); #c1(DIXDC1) c2(NP_001033043) c3(dixin isoform a) c4(31511, 31512) c5(31513, 31514, 31515, 31516, 31517, 31518, 31519, 31520, 31521, 31522, 31523, 31524, 31525, 31526, 31527, 31528, 31529, 31530) c6( )c7(qw, T, bE, bB, bS, ci, ct, Z, bz, q); #c1(DIXDC1) c2(NP_219493) c3(dixin isoform b) c4(228602) c5(228603, 228604, 228605, 228606, 228607, 228608) c6( )c7(qw, T, bE, bB, bS, ci, ct, Z, bz, q); #c1(DIXDC1) c2(XP_005277783) c3 (dixin isoform XI) c4(285750) c5( )c6( )c7(qw, T, bE, bB, bS, ci, ct, Z, bz, q); #c1(DKC1) c2(NP_001354) c3(H/ACA ribonucleoprotein complex subunit 4 isoform 1) c4(1/7251) c5(117252, 117253, 117254, 117255, 117256, 117257, 117258, 117259, 117260) c6( )c7(ck, sb, T, yo, wr, MR, aWZ, rW, lM, Y, aXY, aW, aT, dW, oX, bfG, bt, ae, B, ad); #c1(DKK1) c2(NP_036374) c3(dickkopf-related protein 1 precursor) c4(173339) c5(173340, 173341, 173342, 173343, 173344, 173345) c6( )c7(ng, bM, T, os, hV, atj, e, if, fd, bB, ci, aE, K, hu, H, aW, hl, LZ, g, eF, cm, bb, eM, nB, iA, U, bi, cE, ht, fr, ct, qN, zw, adX, YT, dx, ia, Ln, agF, kp, iO, adZ, dB, Be, fL, bfl, B, vQ, L, AB, lH, aA, bX, q, er, eA, eJ, hb, bfJ, aT, jj, ae, af, eu, bA, jf, oV, Y, r, akY, t, pb, ck, nM, kP, aJt, Bd, bt, gx, aZ, oZ, bS, dG, mC, ad, dP, hd, aet, nJ, d, lR, eH, eZ, aF, Z, bfH, bz, LB, es, zr, bE, atg, y, X); #c1(DKK2) c2(NP_055236) c3(dickkopf-related protein 2 precursor) c4(179984) c5(179985, 179986, 179987, 179988, 179989, 179990, 179991, 179992, 179993) c6(179994, 179995) c7(es, T, aE, iA, Y, qK, fr, m, eA, baU, B, dx); #c1(DKK3) c2(XP_006718241) c3(dickkopf-related protein 3 isoform X1) c4(291590, 291591, 291592, 291593, 291594) c5(291595, 291596, 291597, 291598, 291599, 291600, 291601, 291602, 291603, 291604, 291605, 291606, 291607, 291608, 291609, 291610, 291611, 291612, 291613, 291614, 291615, 291616, 291617, 291618, 291619, 291620, 291621, 291622, 291623, 291624, 291625, 291626, 291627, 291628, 291629, 291630, 291631, 291632, 291633, 291634, 291635, 291636, 291637, 291638, 291639) c6(291640, 291641, 291642, 291643, 291644, 291645, 291646, 291647, 291648, 291649, 291650) c7(cZ, T, aC, Tb, if, aA, bB, fr, ci, qN, q, ad, eA, x, av, hd, py, aE, aT, h, dL, K, dd, ae, eL, jf, d, s, aW, Y, Lf, ff, apn, r, aiT, aq, aF, en, bu, cl, t, g, as, eF, apx, cm, es, iO, nM, eM, nB, bE, iA, hL, nw, dy, m, zl, B, hS); #c1(DKK4) c2(XP_011542791) c3(dickkopf-related protein 4 isoform X1) c4(341417, 341418) c5(341419, 341420, 341421, 341422, 341423, 341424, 341425, 341426, 341427, 341428, 341429, 341430) c6( ) c7(es, T, B, bE, eZ, r, aT, akY, bB, aq, fr, q, cE, X); #c1(DKKL1) c2(NP_001184230) c3(dickkopf-like protein 1 isoform 2 precursor) c4(75571) c5(75572, 75573, 75574, 75575, 75576, 75577) c6( )c7(V); #c1(DKKL1) c2(NP_055234) c3(dickkopf-like protein 1 isoform 1 precursor) c4(179977) c5(179978, 179979, 179980, 179981, 179982, 179983) c6( )c7(V); #c1(DLAT) c2(NP_001922) c3(dihydrolipoyllysine-residue acetyltransferase component of pyruvate dehydrogenase complex, mitochondrial precursor) c4(122217, (22218, 122219) c5(122220, 122221, 122222, 122223, 122224, 122225, 122226, 122227, 122228, 122229, 122230, 122231, 122232) c6( )c7(nZ, sY, Wb, v, ON, bh, j, aNH, bfL, mC, wD, qC, fX, bfK, bc, mR, fc, eZ, sz, uC, pb, FV, fL, cP, jM, hE, bt); #c1(DLAT) c2(XP_011540949) c3(dihydrolipoyllysine-residue acetyltransferase component of pyruvate dehydrogenase complex, mitochondrial isoform X1) c4( ) c5(339477, 339478, 339479, 339480, 339481) c6( )c7(nZ, sY, Wb, v, ON, bh, j, aNH, bfL, mC, wD, qC, fX, bfK, bc, mR, fc, eZ, sz, uC, pb, FV, fL, cP, jM, hE, bt); #c1(DLC1) c2(NP_006085) c3(rho GTPase-activating protein 7 isoform 2) c4(16/784) c5(161785, 161786, 161787, 161788, 161789, 161790, 161791, 161792, 161793, 161794, 161795, 161796, 161797, 161798, 161799, 161800, 161801) c6( )c7(bL, T, abY, iW, jh, aA, bB, n, fd, j, ci, fr, q, ad, ip, hX, av, aAF, aT, io, By, iY, K, hu, hB, ae, jf, B, aW, r, aq, kp, aF, iR, t, ik, bz, ac, eM, bE, m, adg, X); #c1(DLC1) c2(NP_079043) c3(rho GTPase-activating protein 7 isoform 3) c4(216724) c5(216725, 216726, 216727, 216728, 216729, 216730, 216731, 216732, 216733, 216734, 216735, 216736) c6( )c7(bL, T, abY, iW, jh, aA, bB, n, fd, j, ci, fr, q, ad, ip, hX, av, aAF, aT, io, By, iY, K, hu, hB, ae, jf, B, aW, r, aq, kp, aF, iR, t, ik, bz, ac, eM, bE, m, adg, X); #c1(DLC1) c2(NP_872584) c3(rho GTPase-activating protein 7 isoform 1) c4(256418, 256419, 256420, 256421) c5(256422, 256423, 256424, 256425, 256426, 256427, 256428) c6( )c7(bL, T, abY, iW, jh, aA, bB, n, fd, j, ci, fr, q, ad, ip, hX, av, aAF, aT, io, By, iY, K, hu, hB, ae, jf, B, aW, r, aq, kp, aF, iR, t, ik, bz, ac, eM, bE, m, adg, X); #c1(DLD) c2(NP_000099) c3(dihydrolipoyl dehydrogenase, mitochondrial isoform 1 precursor) c4(1757, 1758) c5(1759, 1760, 1761, 1762, 1763, 1764, 1765, 1766, 1767, 1768, 1769, 1770, 1771, 1772, 1773, 1774, 1775) c6( )c7(T, dA, bfM, baD, n, bB, bfN, ci, C, nA, bfO, aNH, q, b, baE, i, dv, gd, Os, cy, agm, K, af, ajU, adn, eG, arl, Cw, oV, ev, aw, r, dq, iV, Kp, arJ, bz, dr, wT, bF, adc, pa, c, eM, bE, Km, lM, X); #c1(DLD) c2(NP_001276679) c3(dihydrolipoyl dehydrogenase, mitochondrial isoform 2) c4(108249) c5(108250, 108251, 108252, 108253, 108254) c6( )c7(T, dA, bfM, baD, n, bB, bfN, ci, C, nA, bfO, aNH, q, b, baE, i, dv, gd, Os, cy, agm, K, af, ajU, adn, eG, arl, Cw, oV, ev, aw, r, dq, iV, Kp, arJ, bz, dr, wT, bF, adc, pa, c, eM, bE, Km, lM, X); #c1(DLD) c2(NP_001276680) c3(dihydrolipoyl dehydrogenase, mitochondrial isoform 3) c4(108255) c5(108256, 108257, 108258, 108259, 108260, 108261) c6( )c7(T, dA, bfM, baD, n, bB, bfN, ci, C, nA, bfO, aNH, q, b, baE, i, dv, gd, Os, cy, agm, K, af, ajU, adn, eG, arl, Cw, oV, ev, aw, r, dq, iV, Kp, arJ, bz, dr, wT, bF, adc, pa, c, eM, bE, Km, lM, X); #c1(DLD) c2(NP_001276681) c3(dihydrolipoyl dehydrogenase, mitochondrial isoform 4) c4(108262) c5(108263, 108264, 108265, 108266, 108267, 108268) c6( )c7(T, dA, bfM, baD, n, bB, bfN, ci, C, nA, bfO, aNH, q, b, baE, i, dv, gd, Os, cy, agm, K, af, ajU, adn, eG, arl, Cw, oV, ev, aw, r, dq, iV, Kp, arJ, bz, dr, wT, bF, adc, pa, c, eM, bE, Km, lM, X); #c1(DLEC1) c2(NP_031361) c3(deleted in lung and esophageal cancer protein 1 isoform DLEC1-N1) c4(171951) c5(171952, 171953, 171954, 171955, 171956, 171957) c6( )c7(GI, T, abY, iW, ht, jh, aA, bB, n, hy, fd, fr, ci, ad, ip, hX, av, aAF, aT, io, Gw, K, hB, ae, jf, aW, ff, aq, kp, aF, en, iR, t, ik, es, eM, bE, Gu, adg, B); #c1(DLEC1) c2(NP_031363) c3(deleted in lung and esophageal cancer protein 1 isoform DLEC1-S3) c4(171958) c5(171959, 171960, 171961, 171962, 171963, 171964) c6( )c7(GI, T, abY, iW, ht, jh, aA, bB, n, hy, fd, fr, ci, ad, ip, hX, av, aAF, aT, io, Gw, K, hB, ae, jf, aW, ff, aq, kp, aF, en, iR, t, iK, es, eM, bE, Gu, adg, B); #c1(DLEC1) c2(XP_005265687) c3(deleted in lung and esophageal cancer protein 1 isoform X2) c4(279669) c5(279670, 279671, 279672, 279673, 279674, 279675) c6( )c7(GI, T, abY, iW, ht, jh, aA, bB, n, hy, fd, fr, ci, ad, ip, hX, av, aAF, aT, io, Gw, K, hB, ae, jf, aW, ff, aq, kp, aF, en, iR, t, ik, es, eM, bE, Gu, adg, B); #c1(DLEU7) c2(NP_945340) c3(leukemia-associated protein 7) c4(260836) c5(260837, 260838, 260839, 260840, 260841, 260842) c6( )c7(x, kT, acW, c, s, SV, cj, cz, j, k, lQ, y, dx); #c1(DLG1) c2(NP_001191315) c3(disks large homolog I isoform 3) c4(81380) c5(81381, 81382, 81383, 81384, 81385, 81386, 81387, 81388, 81389, 81390) c6( )c7(Na, bfP, iM, je, aiB, if, uO, ti, aj, an, he, X, aY, uQ); #c1(DLG1) c2(NP_001191316) c3(disks large homolog 1 isoform 4) c4(81391) c5(81392, 81393, 81394, 81395, 81396, 81397) c6( )c7(Na, bfP, iM, je, aiB, if, ul, ti, aj, an, he, X, aY, uQ); #c1(DLG1) c2(NP_001191317) c3(disks large homolog 1 isoform 5) c4(81398) c5(81399, 81400, 81401, 81402, 81403, 81404) c6( )c7(Na, bfP, iM, je, aiB, if, uO, ti, aj, an, he, X, aY, uQ); #c1(DLG1) c2(NP_001277912) c3(disks large homolog 1 isoform 1) c4(109402, 109403) c5(109404, 109405, 109406, 109407, 109408, 109409, 109410, 109411, 109412, 109413, 109414, 109415, 109416, 109417, 109418, 109419) c6( )c7(Na, bfP, iM, je, aiB, if, ul, ti, aj, an, he, X, aY, u); #c1(DLG1) c2(NP_004078) c3(disks large homolog 1 isoform 2) c4(143367) c5(143368, 143369, 143370, 143371, 143372, 143373) c6( )c7(Na, bfP, iM, je, aiB, if, uO, ti, aj, an, he, X, aY, uQ); #c1(DLG1) c2(XP_011510800) c3(disks large homolog 1 isoform X4) c4( )c5(301194, 301195, 301196, 301197, 301198) c6( )c7(Na, bfP, iM, je, aiB, if, uO, ti, aj, an, he, X, aY, uQ); #c1(DLG1) c2(XP_011510801) c3(disks large homolog 1 isoform X5) c4(301199) c5( )c6( )c7(Na, bfP, iM, je, aiB, if, ul, ti, aj, an, he, X, aY, uQ); #c1(DLG1) c2(XP_011510802) c3(disks large homolog 1 isoform X6) c4(301200) c5(301201, 301202, 301203, 301204, 301205, 301206) c6( )c7(Na, bfP, iM, je, aiB, if, ul, ti, aj, an, he, X, aY, uQ); #c1(DLG1) c2(XP_011510806) c3 (disks large homolog 1 isoform X8) c4(301207) c5( )c6( )c7(Na, bfP, iM, je, aiB, if, ul, ti, aj, an, he, X, aY, uQ); #c1(DLG1) c2(XP_011510807) c3(disks large homolog 1 isoform X9) c4(301208) c5(301209, 301210, 301211, 301212, 301213, 301214) c6( )c7(Na, bfP, iM, je, aiB, if, uO, ti, aj, an, he, X, aY, uQ); #c1(DLG1) c2(XP_011510808) c3(disks large homolog 1 isoform X10) c4(301215) c5(301216, 301217, 301218, 301219, 301220, 301221, 301222, 301223, 301224, 301225) c6( )c7(Na, bfP, iM, je, aiB, if, ul, ti, aj, an, he, X, aY, uQ); #c1(DLG1) c2(XP_011510810) c3(disks large homolog 1 isoform X12) c4( )c5(301226, 301227, 301228, 301229, 301230, 301231) c6( )c7(Na, bfP, iM, je, aiB, if, uO, ti, aj, an, he, X, aY, ul); #c1(DLG1) c2(XP_011510811) c3(disks large homolog 1 isoform X14) c4(301232, 301233, 301234, 301235) c5(301236, 301237, 301238, 301239, 301240, 301241, 301242, 301243, 301244, 301245, 301246, 301247, 301248, 301249, 301250, 301251, 301252, 301253, 301254, 301255, 301256, 301257, 301258, 301259, 301260) c6( )c7(Na, bfP, iM, je, aiB, if, u, ti, aj, an, he, X, aY, uQ); #c1(DLG2) c2(NP_001136172) c3(disks large homolog 2 isoform 3) c4(54472) c5(54473, 54474, 54475, 54476, 54477, 54478, 54479, 54480, 54481) c6( )c7(es, qw, ac, WA, Y, aw, eZ, aT, an, he, cd); #c1(DLG2) c2(NP_001136174) c3(disks large homolog 2 isoform 4) c4( )c5(54482, 54483, 54484, 54485, 54486, 54487, 54488, 54489, 54490, 54491, 54492, 54493, 54494, 54495, 54496, 54497, 54498, 54499) c6( )c7(es, qw, ac, WA, Y, aw, eZ, aT, an, he, cd); #c1(DLG2) c2(NP_001193698) c3(disks large homolog 2 isoform 5) c4(82478) c5(82479, 82480, 82481, 82482, 82483, 82484) c6( )c7(es, qw, ac, WA, Y, aw, eZ, aT, an, he, cd); #c1(DLG2) c2(NP_001287912) c3(disks large homolog 2 isoform 6) c4(113184, 113185) c5(113186, 113187, 113188, 113189, 113190, 113191, 113192, 113193, 113194, 113195, 113196, 113197) c6( )c7(es, qw, ac, WA, Y, aw, eZ, aT, an, he, cd); #c1(DLG2) c2(NP_001355) c3(disks large homolog 2 isoform 2) c4(117261) c5(117262, 117263, 117264, 117265, 117266, 117267) c6( )c7(es, qw, ac, WA, Y, aw, eZ, aT, an, he, cd); #c1(DLG2) c2(XP_005273868) c3(disks large homolog 2 isoform X11) c4(284861) c5(284862, 284863, 284864, 284865, 284866, 284867, 284868, 284869, 284870, 284871, 284872, 284873) c6( )c7(es, qw, ac, WA, Y, aw, eZ, aT, an, he, cd); #c1(DLG2) c2(XP_011543081) c3(disks large homolog 2 isoform X2) c4(341905, 341906) c5(341907, 341908, 341909, 341910, 341911, 341912, 341913, 341914, 341915, 341916, 341917, 341918, 341919, 341920, 341921, 341922) c6( )c7(es, qw, ac, WA, Y, aw, eZ, aT, an, he, cd); #c1(DLG2) c2(XP_011543082) c3(disks large homolog 2 isoform X3) c4(341923, 341924, 341925) c5( )c6( )c7(es, qw, ac, WA, Y, aw, eZ, aT, an, he, cd); #c1(DLG2) c2(XP_011543088) c3(disks large homolog 2 isoform X12) c4(341926) c5( )c6( )c7(es, qw, ac, WA, Y, aw, eZ, aT, an, he, cd); #c1(DLG3) c2(NP_001159750) c3(disks large homolog 3 isoform c) c4(64700) c5(64701, 64702, 64703, 64704, 64705) c6( )c7(bF, T, nc, d, cA, bfQ, hb, FB, nd, bO, qy, nf, n, dr, aU, bK, nA, Ku, aR, bN); #c1(DLG3) c2(NP_065781) c3(disks large homolog 3 isoform b) c4(205405) c5(205406, 205407, 205408, 205409, 205410, 205411, 205412) c6( )c7(bF, T, nc, d, cA, bfQ, hb, FB, nd, bO, qy, nf, n, dr, aU, bK, nA, Ku, aR, bN); #c1(DLG3) c2(NP_066943) c3(disks large homolog 3 isoform a) c4(207637) c5(207638, 207639, 207640, 207641, 207642, 207643) c6( )c7(bF, T, nc, d, cA, bfQ, hb, FB, nd, bO, qy, nf, n, dr, aU, bk, nA, Ku, aR, bN); #c1(DLG3) c2(XP_006724689) c3(disks large homolog 3 isoform X4) c4(296254) c5(296255, 296256, 296257, 296258, 296259, 296260) c6( )c7(bF, T, nc, d, cA, bfQ, hb, FB, nd, bQ, qy, nf, n, dr, aU, bK, nA, Ku, aR, bN); #c1(DLG4) c2(NP_001122299) c3(disks large homolog 4 isoform 2) c4(46465) c5(46466, 46467, 46468, 46469, 46470, 46471) c6( )c7(ha, bO, il, Hx, XY, bK, bfR, an, rE, af, kl, VX, aFx, cm, ek, bN, hq, rR, alC, cA, U, od, qc); #c1(DLG4) c2(NP_001356) c3(disks large homolog 4 isoform 1 precursor) c4(117268, 117269, 117270) c5(117271, 117272, 117273, 117274, 117275, 117276, 117277, 117278, 117279, 117280, 117281, 117282, 117283, 117284, 117285, 117286, 117287, 117288) c6( )c7(ha, bO, il, Hx, XY, bK, bfR, an, rE, af, kl, VX, aFx, cm, ek, bN, hq, rR, alC, cA, U, oQ, qc); #c1(DLG4) c2(XP_005256549) c3(disks large homolog 4 isoform X5) c4(274195) c5(274196, 274197, 274198, 274199, 274200, 274201, 274202) c6( )c7(ha, bO, il, Hx, XY, bK, bfR, an, rE, af, kl, VX, aFx, cm, ek, bN, hq, rR, alC, cA, U, od, qc); #c1(DLG4) c2(XP_011522004) c3(disks large homolog 4 isoform X6) c4(316689) c5(316690, 316691, 316692, 316693, 316694) c6( )c7(ha, bO, il, Hx, XY, bK, bfR, an, rE, af, kl, VX, aFx, cm, ek, bN, hq, rR, alC, cA, U, oQ, qc); #c1(DLG5) c2(NP_004738) c3(disks large homolog 5) c4(149/79) c5(149180, 149181, 149182, 149183, 149184, 149185, 149186, 149187, 149188, 149189, 149190, 149191) c6( )c7(ul, aC, oe, r, fe, hN, alO, kp, hp, iV, y, ra); #c1(DLG5) c2(XP_006725185) c3(disks large homolog 5 isoform X9) c4(296703, 296704) c5(296705, 296706, 296707, 296708, 296709, 296710, 296711, 296712, 296713, 296714, 296715, 296716) c6( ) c7(ul, aC, oe, r, fe, hN, alO, kp, hp, iV, y, ra); #c1(DLG5) c2(XP_006725186) c3(disks large homolog 5 isoform X10) c4( )c5(296717) c6( ) c7(ul, aC, oe, r, fe, hN, alO, kp, hp, iV, y, ra); #c1(DLG5) c2(XP_011544879) c3(disks large homolog 5 isoform X5) c4(343867) c5( )c6( ) c7(ul, aC, oe, r, fe, hN, alO, kp, hp, iV, y, ra); #c1(DLGAP1) c2(NP_001003809) c3(disks large-associated protein 1 isoform 2) c4(20162) c5(20163, 20164, 20165, 20166, 20167, 20168) c6( )c7(c, pN, j, Ea, cm, fq, cd); #c1(DLGAP1) c2(NP_001229690) c3(disks large-associated protein 1 isoform 3) c4(84787) c5(84788, 84789, 84790, 84791, 84792, 84793, 84794, 84795, 84796, 84797) c6( )c7(c, pN, j, Ea, cm, fq, cd); #c1(DLGAP1) c2(NP_001229691) c3(disks large-associated protein 1 isoform 4) c4(84798) c5(84799, 84800, 84801, 84802, 84803, 84804) c6( )c7(c, pN, j, Ea, cm, fq, cd); #c1(DLGAP1) c2(NP_001229693) c3(disks large-associated protein 1 isoform 6) c4(84805) c5(84806, 84807, 84808, 84809, 84810, 84811) c6( )c7(c, pN, j, Ea, cm, fq, cd); #c1(DLGAP1) c2(NP_001229694) c3 (disks large-associated protein 1 isoform 7) c4(84812) c5(84813, 84814, 84815, 84816, 84817, 84818) c6( )c7(c, pN, j, Ea, cm, fq, cd); #c1(DLGAP1) c2(NP_001229695) c3(disks large-associated protein 1 isoform 8) c4(84819) c5(84820, 84821, 84822, 84823, 84824, 84825) c6( )c7(c, pN, j, Ea, cm, fq, cd); #c1(DLGAP1) c2(NP_004737) c3(disks large-associated protein 1 isoform 1) c4(149172) c5(149/73, 149174, 149175, 149176, 149177, 149178) c6( )c7(c, pN, j, Ea, cm, fq, cd); #c1(DLGAP2) c2(XP_011533064) c3(disks large-associated protein 2 isoform X1) c4(329275) c5(329276, 329277, 329278, 329279, 329280) c6( )c7(bN, alQ, aPS); #c1(DLGAP2) c2(XP_011545673) c3(disks large-associated protein 2 isoform X6) c4( )c5(344584) c6( )c7(bN, alQ, aPS); #c1(DLGAP2) c2(XP_011545674) c3(disks large-associated protein 2 isoform X7) c4(344585, 344586) c5(344587, 344588, 344589, 344590, 344591, 344592, 344593, 344594, 344595, 344596, 344597, 344598) c6( )c7(bN, alQ, aPS); #c1(DLGAP3) c2(XP_011540181) c3(disks large-associated protein 3 isoform X1) c4(338568, 338569) c5(338570, 338571, 338572, 338573, 338574, 338575, 338576, 338577, 338578, 338579, 338580, 338581, 338582, 338583, 338584, 338585) c6( )c7(d, nB, yL, aA, aF, rE, pd, eA, qc); #c1(DLGAP5) c2(NP_001139487) c3(disks large-associated protein 5 isoform b) c4(59427) c5(59428, 59429, 59430, 59431, 59432, 59433) c6( ) c7(T, iM, je, aiB, eL, iw, uO, r, n, dL, ey, q, cm, ae, B, ad, ul); #c1(DLGAP5) c2(XP_011535716) c3(disks large-associated protein 5 isoform XI) c4(332954) c5(332955, 332956, 332957, 332958, 332959, 332960) c6( )c7(T, iM, je, aiB, eL, iw, uO, r, n, dL, ey, q, cm, ae, B, ad, uQ); #c1(DLK1) c2(NP_003827) c3(protein delta homolog 1 precursor) c4(141185, 141186) c5(141187, 141188, 141189, 141190, 141191, 141192) c6( )c7(gV, T, os, fM, dA, bM, Tb, aw, ml, jh, bs, aA, cz, mw, v, oZ, ci, fr, rc, ad, br, Nh, er, eA, AW, bv, mj, x, pX, KH, iv, h, iY, vB, jj, RG, ae, bfT, bfS, yo, A, d, Ar, hG, il, Hh, bg, Pg, aLJ, eZ, kt, aq, fe, aF, hp, Fq, iV, iR, cm, nC, bw, pb, Cv, ck, ba, es, iO, pc, iA, dB, bE, SC, hR, pg, St, Xu, eg, aps, bt, ra, B, y); #c1(DLL1) c2(XP_011534060) c3(delta-like protein 1 isoform X1) c4(330245) c5(330246, 330247, 330248, 330249, 330250, 330251, 330252, 330253) c6(330254, 330255) c7(bM, T, bB, ci, afz, x, AW, pp, iX, RG, bw, qJ, iA, avS, iT, aDs, cj, St, k, pw, eg, fr, br, Nh, kH, h, hn, ZW, fe, bfW, adz, hq, iO, cA, dB, bfX, bL, aA, cz, v, bfU, V, eA, ady, aql, aT, cy, af, il, xR, Y, Pg, aLJ, lo, nC, pb, Cv, ck, BU, bt, gV, bfY, jh, bs, mw, bS, bck, fu, hF, hX, ds, bfZ, vB, hG, bfV, eZ, aF, hp, iV, bz, es, bE, SC, y, ra, EB); #c1(DLL3) c2(NP_058637) c3(delta-like protein 3 isoform 1 precursor) c4(192897, 192898) c5(192899, 192900, 192901, 192902, 192903, 192904, 192905) c6( ) c7(T, bga, qb, qx, fd, ci, bfU, VV, eA, eW, x, A1, aT, qv, aNZ, on, rv, Gc, aW, Az, aeS, hp, zC, aoD, as, bl, bw, es, lq, mx, cj, k, bt, B); #c1(DLL3) c2(NP_982353) c3(delta-like protein 3 isoform 2 precursor) c4( )c5(263362, 263363, 263364, 263365, 263366, 263367, 263368) c6( )c7(T, bga, qb, qx, fd, ci, bfU, W, eA, eW, x, A1, aT, qv, aNZ, on, rv, Gc, aW, Az, aeS, hp, zC, aoD, as, bl, bw, es, lq, mx, cj, k, bt, B); #c1(DLL4) c2(NP_061947) c3(delta-like protein 4 precursor) c4(201251) c5(201252, 201253, 201254, 201255, 201256, 201257) c6( )c7(T, aZ, ha, aA, bB, bck, lN, ci, mF, V, q, cd, ad, eA, qC, ft, cq, aE, py, akJ, bfZ, adX, aT, hD, iv, dL, nJ, ae, eL, jK, cY, jf, DA, kl, d, aMG, aW, fc, hl, r, nf, dq, lo, kp, aF, yq, t, bz, ck, es, eM, adZ, lI, bE, iA, k, m, ym, hh, hE, bt, X); #c1(DLST) c2(NP_001924) c3(dihydrolipoyllysine-residue succinyltransferase component of 2-oxoglutarate dehydrogenase complex, mitochondrial isoform 1 precursor) c4(122239, 122240) c5(122241, 122242, 122243, 122244, 122245, 122246, 122247, 122248, 122249, 122250, 122251, 122252) c6( )c7(Gm, af, U, bc); #c1(DLX1) c2(NP_001033582) c3(homeobox protein DLX-1 isoform 2) c4(31545) c5(31546, 31547, 31548, 31549, 31550, 31551) c6( )c7(VX, ha, Az, gl, bK, rz, bN); #c1(DLXI) c2(NP_835221) c3(homeobox protein DLX-1 isoform 1) c4(253368, 253369, 253370) c5(253371, 253372, 253373, 253374, 253375, 253376, 253377, 253378, 253379, 253380, 253381, 253382, 253383, 253384, 253385, 253386, 253387, 253388) c6( )c7(VX, ha, Az, gl, bK, rz, bN); #c1(DLX2) c2(NP_004396) c3(homeobox protein DLX-2) c4(146073, 146074) c5(146075, 146076, 146077, 146078, 146079, 146080, 146081, 146082, 146083, 146084, 146085, 146086, 146087) c6( )c7(x, T, anA, aW, ha, Az, aT, anz, K, bN); #c1(DLX3) c2(NP_005211) c3(homeobox protein DLX-3) c4(154056) c5(154057, 154058, 154059, 154060, 154061, 154062, 154063) c6( )c7(bgb, pB, bgc, Ud, nJ); #c1(DLX4) c2(NP_612138) c3(homeobox protein DLX-4 isoform a) c4(236001) c5(236002, 236003, 236004, 236005, 236006, 236007) c6( )c7(ck, av, BU, iH, aW, ff, aT, bK, m, R, so, en, t, nJ, pB, ad, hq); #c1(DLX4) c2(XP_011522761) c3(homeobox protein DLX-4 isoform X1) c4(317275) c5(317276, 317277, 317278, 317279, 317280, 317281, 317282) c6( ) c7(ck, av, BU, iH, aW, ff, aT, bK, m, R, so, en, t, nJ, pB, ad, hq); #c1(DLX5) c2(NP_005212) c3(homeobox protein DLX-5) c4(154064) c5(154065, 154066, 154067, 154068, 154069, 154070, 154071) c6( )c7(T, jh, Na, av, arA, vJ, il, aT, aOV, aW, Az, t, bz, zV, bN, bge, Di, eM, BP, mG, bgd, m); #c1(DLX6) c2(NP_005213) c3(homeobox protein DLX-6) c4(154072, 154073) c5(154074, 154075, 154076, 154077, 154078, 154079, 154080, 154081, 154082, 154083, 154084, 154085, 154086, 154087, 154088) c6( )c7(Na, Qi, arA, vJ, BP, il, Az, bgd, alV, atb, bN); #c1(DMBT1) c2(NP_004397) c3(deleted in malignant brain tumors 1 protein isoform a precursor) c4(146088) c5(146089, 146090, 146091, 146092, 146093, 146094, 146095, 146096, 146097, 146098, 146099, 146100, 146101, 146102, 146103) c6( )c7(Mb, ka, T, tp, yk, e, al, LX, qy, aA, bB, ci, Fc, q, ad, eA, hX, XX, ft, jz, aE, aT, h, cy, dL, ji, K, ae, bc, eL, jf, jr, A, d, B, s, aW, Y, ff, bgh, hl, aft, r, fe, bgf, af, EZ, en, g, as, cm, iV, bz, Dm, bgg, gk, eM, dB, bE, ki, qr, lf, gX, wR, HE, X, LT); #c1(DMBT1) c2(NP_060049) c3(deleted in malignant brain tumors 1 protein isoform c precursor) c4(193877) c5(193878, 193879, 193880, 193881, 193882, 193883, 193884, 193885, 193886) c6( ) c7(Mb, ka, T, tp, yk, e, al, LX, qy, aA, bB, ci, Fc, q, ad, eA, hX, XX, ft, jz, aE, aT, h, cy, dL, ji, K, ae, bc, eL, jf, jr, A, d, B, s, aW, Y, ff, bgh, hl, aFt, r, fe, bgf, aF, EZ, en, g, as, cm, iV, bz, Dm, bgg, gk, eM, dB, bE, ki, qr, lf, gX, wR, HE, X, LT); #c1(DMBT1) c2(XP_011537690) c3(deleted in malignant brain tumors 1 protein isoform X1) c4(335231, 335232) c5(335233, 335234, 335235, 335236, 335237, 335238) c6( )c7(Mb, ka, T, tp, yk, e, al, LX, qy, aA, bB, ci, Fc, q, ad, eA, hX, XX, ft, jz, aE, aT, h, cy, dL, ji, K, ae, bc, eL, jf, jr, A, d, B, s, aW, Y, ff, bgh, hl, aFt, r, fe, bgf, aF, EZ, en, g, as, cm, iV, bz, Dm, bgg, gk, eM, dB, bE, ki, qr, lf, gX, wR, HE, X, LT); #c1(DMBT1) c2(XP_011537707) c3 (deleted in malignant brain tumors 1 protein isoform X20) c4(335239) c5(335240, 335241, 335242, 335243, 335244, 335245, 335246, 335247, 335248, 335249, 335250, 335251, 335252, 335253, 335254, 335255, 335256, 335257) c6( )c7(Mb, ka, T, tp, yk, e, al, LX, qy, aA, bB, ci, Fc, q, ad, eA, hX, XX, ft, jz, aE, aT, h, cy, dL, ji, K, ae, bc, eL, jf, jr, A, d, B, s, aW, Y, ff, bgh, hl, aFt, r, fe, bgf, aF, EZ, en, g, as, cm, iV, bz, Dm, bgg, gk, eM, dB, bE, ki, qr, lf, gX, wR, HE, X, LT); #c1 (DMBT1) c2(XP_011537708) c3(deleted in malignant brain tumors 1 protein isoform X21) c4(335258) c5(335259, 335260, 335261, 335262, 335263, 335264) c6( )c7(Mb, ka, T, tp, yk, e, al, LX, qy, aA, bB, ci, Fc, q, ad, eA, hX, XX, ft, jz, aE, aT, h, cy, dL, ji, K, ae, bc, eL, jf, jr, A, d, B, s, aW, Y, ff, bgh, hl, aFt, r, fe, bgf, aF, EZ, en, g, as, cm, iV, bz, Dm, bgg, gk, eM, dB, bE, ki, qr, lf, gX, wR, HE, X, LT); #c1(DMBT1) c2(XP_011537712) c3(deleted in malignant brain tumors 1 protein isoform X25) c4(335265) c5(335266, 335267, 335268, 335269, 335270, 335271) c6( )c7(Mb, ka, T, tp, yk, e, al, LX, qy, aA, bB, ci, Fc, q, ad, eA, hX, XX, ft, jz, aE, aT, h, cy, dL, ji, K, ae, bc, eL, jf, jr, A, d, B, s, aW, Y, ff, bgh, hl, aFt, r, fe, bgf, aF, EZ, en, g, as, cm, iV, bz, Dm, bgg, gk, eM, dB, bE, ki, qr, lf, gX, wR, hE, X, LT); #c1(DMBT1) c2(XP_011537713) c3(deleted in malignant brain tumors 1 protein isoform X26) c4( )c5(335272) c6( )c7(Mb, ka, T, tp, yk, e, al, LX, qy, aA, bB, ci, Fc, q, ad, eA, hX, XX, ft, jz, aE, aT, h, cy, dL, ji, K, ae, bc, eL, jf, jr, A, d, B, s, aW, Y, ff, bgh, hl, aFt, r, fe, bgf, aF, EZ, en, g, as, cm, iV, bz, Dm, bgg, gk, eM, dB, bE, ki, qr, lf, gX, wR, HE, X, LT); #c1(DMBT1) c2(XP_011537717) c3(deleted in malignant brain tumors 1 protein isoform X30) c4( )c5(335273, 335274, 335275, 335276, 335277, 335278) c6( )c7(Mb, ka, T, tp, yk, e, al, LX, qy, aA, bB, ci, Fc, q, ad, eA, hX, XX, ft, jz, aE, aT, h, cy, dl, ji, K, ae, bc, eL, jf, jr, A, d, B, s, aW, Y, ff, bgh, hl, aft, r, fe, bgf, aF, EZ, en, g, as, cm, iV, bz, Dm, bgg, gk, eM, dB, bE, ki, qr, lf, gX, wR, HE, X, LT); #c1(DMC1) c2(NP_008999) c3(meiotic recombination protein DMC1/LIM15 homolog isoform 1) c4(170156, 170157) c5(170158, 170159, 170160, 170161, 170162, 170163, 170164, 170165, 170166, 170167, 170168, 170169) c6( )c7(T, ND, il, aW, aT, vH, zZ); #c1(DMC1) c2(XP_006724175) c3(meiotic recombination protein DMC1/LIM15 homolog isoform X1) c4(295830, 295831) c5(295832, 295833, 295834, 295835, 295836, 295837, 295838, 295839, 295840, 295841, 295842, 295843, 295844, 295845, 295846, 295847, 295848, 295849) c6( )c7(T, ND, il, aW, aT, vH, zZ); #c1(DMC1) c2(XP_011528137) c3(meiotic recombination protein DMC1/LIM15 homolog isoform X2) c4(323257) c5( )c6( )c7(T, ND, il, aW, aT, vH, zZ); #c1(DMD) c2(NP_000100) c3(dystrophin Dp427c isoform) c4(1776) c5(1777, 1778, 1779, 1780, 1781, 1782, 1783, 1784, 1785, 1786) c6( )c7(T, ea, agU, bgs, afl, HU, bgl, aE, aDk, il, fm, ari, bgm, bgw, K, amA, hm, are, lW, uY, bp, pl, eY, Kc, aeB, bgu, mx, U, cj, nw, mk, m, lS, bgi, ayw, dD, Ol, aFc, Ri, Bm, n, aj, jA, nA, cd, afR, b, no, cc, pN, adX, Ak, afV, arc, bl, he, on, bgn, Hz, lb, Ad, mo, jT, fq, cA, adZ, dB, oh, rx, aiS, arg, cB, bgv, bL, cz, arw, j, bgq, eA, J, bgk, hW, Wn, kf, cy, aWs, bK, zp, yj, Wq, bA, bgp, VC, kl, aqZ, Or, oV, Y, lo, rR, c, aqu, km, arf, ao, aof, hS, arh, bgj, eq, Rk, yt, sO, AB, vJ, oj, Ps, bgr, fb, anR, xz, xs, hp, Bu, bz, dr, bgt, dR, Bv, yE, eB, bgo, ll, gZ, oQ, i, y, xg, jr); #c1(DMD) c2(NP_003997) c3(dystrophin Dp427m isoform) c4(142710, 142711) c5(142712, 142713, 142714, 142715, 142716, 142717, 142718, 142719, 142720, 142721, 142722, 142723, 142724, 142725, 142726, 142727, 142728, 142729, 142730, 142731, 142732, 142733, 142734, 142735, 142736, 142737, 142738) c6( )c7(T, ea, agU, bgs, afl, HU, bgl, aE, aDk, il, fm, ari, bgm, bgw, K, amA, hm, are, lW, uY, bp, pl, eY, Kc, aeB, bgu, mx, U, cj, nw, mk, m, lS, bgi, ayw, dD, OI, aFc, Ri, Bm, n, aj, jA, nA, cd, afR, b, no, cc, pN, adX, Ak, afV, arc, bl, he, on, bgn, Hz, lb, Ad, mo, jT, fq, cA, adZ, dB, oh, rx, aiS, arg, cB, bgv, bL, cz, arw, j, bgq, eA, J, bgk, hW, Wn, kf, cy, aWs, bK, zp, yj, Wq, bA, bgp, VC, kl, aqZ, Ur, oV, Y, lo, rR, c, aqu, km, arf, ao, aof, hS, arh, bgj, eq, Rk, yt, sO, Ae, vJ, oj, Ps, bgr, fb, anR, xz, xs, hp, Bu, bz, dr, bgt, dR, Bv, yE, eB, bgo, ll, gZ, ol, i, y, xg, jr); #c1(DMD) c2(NP_004000) c3(dystrophin Dp427pl isoform) c4(142739, 142740, 142741) c5(142742, 142743, 142744, 142745, 142746, 142747, 142748, 142749) c6( ) c7(T, ea, agU, bgs, afl, HU, bgl, aE, aDk, il, fm, ari, bgm, bgw, K, amA, hm, are, lW, uY, bp, pl, eY, Kc, aeB, bgu, mx, U, cj, nw, mk, m, lS, bgi, ayw, dD, Ol, aFc, Ri, Bm, n, aj, jA, nA, cd, afR, b, no, cc, pN, adX, Ak, afV, arc, bl, he, on, bgn, Hz, lb, Ad, mo, jT, fq, cA, adZ, dB, oh, rx, ais, arg, cB, bgv, bL, cz, arw, j, bgq, eA, J, bgk, hW, Wn, kf, cy, aWs, bK, zp, yj, Wq, bA, bgp, VC, kl, aqZ, Qr, oV, Y, lo, rR, c, aqu, km, arf, ao, aof, hS, arh, bgj, eq, Rk, yt, sO, Ae, vJ, oj, Ps, bgr, fb, anR, xz, xs, hp, Bu, bz, dr, bgt, dR, Bv, yE, eB, bgo, ll, gZ, ol, i, y, xg, jr); #c1(DMD) c2(NP_004001) c3(dystrophin Dp427p2 isoform) c4(142750) c5( )c6( )c7(T, ea, agU, bgs, afl, HU, bgl, aE, aDk, il, fm, ari, bgm, bgw, K, amA, hm, are, lW, uY, bp, pl, eY, Kc, aeB, bgu, mx, U, cj, nw, mk, m, lS, bgi, ayw, dD, Ol, aFc, Ri, Bm, n, aj, jA, nA, cd, afR, b, no, cc, pN, adX, Ak, afV, arc, bl, he, on, bgn, Hz, lb, Ad, mo, jT, fq, cA, adZ, dB, oh, rx, aiS, arg, cB, bgv, bL, cz, arw, j, bgq, eA, J, bgk, hW, Wn, kf, cy, aWs, bK, zp, yj, Wq, bA, bgp, VC, kl, aqZ, Qr, oV, Y, lo, rR, c, aqu, km, arf, ao, aof, hS, arh, bgj, eq, Rk, yt, sO, Ae, vJ, oj, Ps, bgr, fb, anR, xz, xs, hp, Bu, bz, dr, bgt, dR, Bv, yE, eB, bgo, ll, gZ, oQ, i, y, xg, jr); #c1(DMD) c2(NP_004002) c3(dystrophin Dp260-1 isoform) c4(142751) c5(142752, 142753, 142754, 142755, 142756, 142757, 142758) c6( )c7(T, ea, agU, bgs, afl, HU, bgl, aE, aDk, il, fm, ari, bgm, bgw, K, amA, hm, are, lW, uY, bp, pl, eY, Kc, aeB, bgu, mx, U, cj, nw, mk, m, lS, bgi, ayw, dD, Ol, aFc, Ri, Bm, n, aj, jA, nA, cd, afR, b, no, cc, pN, adX, Ak, afV, arc, bl, he, on, bgn, Hz, lb, Ad, mo, jT, fq, cA, adZ, dB, oh, rx, aiS, arg, cB, bgv, bL, cz, arw, j, bgq, eA, J, bgk, hW, Wn, kf, cy, aWs, bK, zp, yj, Wq, bA, bgp, VC, kl, aqZ, Qr, oV, Y, lo, rR, c, aqu, km, arf, ao, aof, hS, arh, bgj, eq, Rk, yt, sO, Ae, vJ, oj, Ps, bgr, fb, anR, xz, xs, hp, Bu, bz, dr, bgt, dR, Bv, yE, eB, bgo, ll, gZ, oQ, i, y, xg, jr); #c1(DMD) c2(NP_004003) c3(dystrophin Dp260-2 isoform) c4(142759) c5(142760, 142761, 142762, 142763, 142764, 142765, 142766) c6( )c7(T, ea, agU, bgs, afl, HU, bgl, aE, aDk, il, fm, ari, bgm, bgw, K, amA, hm, are, lW, uY, bp, pl, eY, Kc, aeB, bgu, mx, U, cj, nw, mk, m, lS, bgi, ayw, dD, Ol, aFc, Ri, Bm, n, aj, jA, nA, cd, afR, b, no, cc, pN, adX, Ak, afV, arc, bl, he, on, bgn, Hz, lb, Ad, mo, jT, fq, cA, adZ, dB, oh, rx, aiS, arg, cB, bgv, bL, cz, arw, j, bgq, eA, J, bgk, hW, Wn, kf, cy, aWs, bK, zp, yj, Wq, bA, bgp, VC, kl, aqZ, Or, oV, Y, lo, rR, c, aqu, km, arf, ao, aof, hS, arh, bgj, eq, Rk, yt, sO, Ae, vJ, oj, Ps, bgr, fb, anR, xz, xs, hp, Bu, bz, dr, bgt, dR, Bv, yE, eB, bgo, ll, gZ, oQ, i, y, xg, jr); #c1(DMD) c2(NP_004005) c3(dystrophin Dp116 isoform) c4( )c5(142767) c6( )c7(T, ea, agU, bgs, afl, HU, bgl, aE, aDk, il, fm, ari, bgm, bgw, K, amA, hm, are, lW, uY, bp, pL, eY, Kc, aeB, bgu, mx, U, cj, nw, mk, m, lS, bgi, ayw, dD, Ol, aFc, Ri, Bm, n, aj, jA, nA, cd, afR, b, no, cc, pN, adX, Ak, afV, arc, bl, he, on, bgn, Hz, lb, Ad, mo, jT, fq, cA, adZ, dB, oh, rx, aiS, arg, cB, bgv, bL, cz, arw, j, bgq, eA, J, bgk, hW, Wn, kf, cy, aWs, bK, zp, yj, Wq, bA, bgp, VC, kl, aqZ, Or, oV, Y, lo, rR, c, aqu, km, arf, ao, aof, hS, arh, bgj, eq, Rk, yt, sO, Ae, vJ, oj, Ps, bgr, fb, anR, xz, xs, hp, Bu, bz, dr, bgt, dR, Bv, yE, eB, bgo, ll, gZ, ol, i, y, xg, jr); #c1(DMD) c2(NP_004006) c3(dystrophin Dp71 isoform) c4( )c5(142768, 142769, 142770, 142771, 142772, 142773, 142774, 142775) c6( )c7(T, ea, agU, bgs, afl, HU, bgl, aE, aDk, il, fm, ari, bgm, bgw, K, amA, hm, are, lW, uY, bp, pl, eY, Kc, aeB, bgu, mx, U, cj, nw, mk, m, lS, bgi, ayw, dD, Ol, aFc, Ri, Bm, n, aj, jA, nA, cd, afR, b, no, cc, pN, adX, Ak, afV, arc, bl, he, on, bgn, Hz, lb, Ad, mo, jT, fq, cA, adZ, dB, oh, rx, aiS, arg, cB, bgv, bL, cz, arw, j, bgq, eA, J, bgk, hW, Wn, kf, cy, aWs, bK, zp, yj, Wq, bA, bgp, VC, kl, aqZ, Qr, oV, Y, lo, rR, c, aqu, km, arf, ao, aof, hS, arh, bgj, eq, Rk, yt, sO, Ae, vJ, oj, Ps, bgr, fb, anR, xz, xs, hp, Bu, bz, dr, bgt, dR, Bv, yE, eB, bgo, ll, gZ, ol, i, y, xg, jr); #c1(DMD) c2(NP_004007) c3(dystrophin Dp71b isoform) c4( )c5(142776, 142777, 142778, 142779, 142780, 142781, 142782, 142783) c6( )c7(T, ea, agU, bgs, afl, HU, bgl, aE, aDk, il, fm, ari, bgm, bgw, K, amA, hm, are, lW, uY, bp, pl, eY, Kc, aeB, bgu, mx, U, cj, nw, mk, m, lS, bgi, ayw, dD, Ol, aFc, Ri, Bm, n, aj, jA, nA, cd, afR, b, no, cc, pN, adX, Ak, afV, arc, bl, he, on, bgn, Hz, lb, Ad, mo, jT, fq, cA, adZ, dB, oh, rx, aiS, arg, cB, bgv, bL, cz arw, j, bgq, eA, J, bgk, hW, Wn, kf, cy, aWs, bK, zp, yj, Wq, bA, bgp, VC, kl, aqZ, Qr, oV, Y, lo, rR, c, aqu, km, arf, ao, aof, hS, arh, bgj, eq, Rk, yt, sO, Ae, vJ, oj, Ps, bgr, fb, anR, xz, xs, hp, Bu, bz, dr, bgt, dR, Bv, yE, eB, bgo, ll, gZ, ol, i, y, xg, jr); #c1(DMD) c2(NP_004008) c3(dystrophin Dp71a isoform) c4( )c5(142784) c6( )c7(T, ea, agU, bgs, afl, HU, bgl, aE, aDk, il, fm, ari, bgm, bgw, K, amA, hm, are, lW, uY, bp, p, eY, Kc, aeB, bgu, mx, U, cj, nw, mk, m, lS, bgi, ayw, dD, Ol, aFc, Ri, Bm, n, aj, jA, nA, cd, afR, b, no, cc, pN, adX, Ak, afV, arc, bl, he, on, bgn, Hz, lb, Ad, mo, jT, fq, cA, adZ, dB, oh, rx, aiS, arg, cB, bgv, bL, cz, arw, j, bgq, eA, J, bgk, hW, Wn, kf, cy, aWs, bK, zp, yj, Wq, bA, bgp, VC, kl, aqZ, Ar, oV, Y, lo, rR, c, aqU, km, arf, ao, aof, hS, arh, bgj, eq, Rk, yt, sO, Ae, vJ, oj, Ps, bgr, fb, anR, xz, xs, hp, Bu, bz, dr, bgt, dR, Bv, yE, eB, bgo, ll, gZ, ol, i, y, xg, jr); #c1(DMD) c2(NP_004009) c3(dystrophin Dp7lab isoform) c4( )c5(142785, 142786, 142787, 142788, 142789, 142790, 142791, 142792) c6( )c7(T, ea, agU, bgs, afl, HU, bgl, aE, aDk, il, fm, ari, bgm, bgw, K, amA, hm, are, lW, uY, bp, pl, eY, Kc, aeB, bgu, mx, U, cj, nw, mk, m, lS, bgi, ayw, dD, Ol, aFc, Ri, Bm, n, aj, jA, nA, cd, afR, b, no, cc, pN, adX, Ak, afV, arc, bl, he, on, bgn, Hz, lb, Ad, mo, jT, fq, cA, adZ, dB, oh, rx, aiS, arg, cB, bgv, bL, cz, arw, j, bgq, eA, J, bgk, hW, Wn, kf, cy, aWs, bK, zp, yj, Wq, bA, bgp, VC, kl, aqZ, Qr, oV, Y, lo, rR, c, aqu, km, arf, ao, aof, hS, arh, bgj, eq, Rk, yt, sO, Ae, vJ, oj, Ps, bgr, fb, anR, xz, xs, hp, Bu, bz, dr, bgt, dR, Bv, yE, eB, bgo, ll, gZ, oQ, i, y, xg, jr); #c1(DMD) c2(NP_004010) c3(dystrophin Dp40 isoform) c4( )c5(142793) c6( )c7(T, ea, agU, bgs, afl, HU, bgl, aE, aDk, il, fm, ari, bgm, bgw, K, amA, hm, are, lW, uY, bp, pl, eY, Kc, aeB, bgu, mx, U, cj, nw, mk, m, lS, bgi, ayw, dD, Ol, aFc, Ri, Bm, n, aj, jA, nA, cd, afR, b, no, cc, pN, adX, Ak, afV, arc, bl, he, on, bgn, Hz, lb, Ad, mo, jT, fq, cA, adZ, dB, oh, rx, aiS, arg, cB, bgv, bL, cz, arw, j, bgq, eA, J, bgk, hW, Wn, kf, cy, aWs, bK, zp, yj, Wq, bA, bgp, VC, kl, aqZ, Dr, oV, Y, lo, rR, c, aqU, km, arf, ao, aof, hS, arh, bgj, eq, Rk, yt, sO, Ae, vJ, oj, Ps, bgr, fb, anR, xz, xs, hp, Bu, bz, dr, bgt, dR, Bv, yE, eB, bgo, ll, gZ, oQ, i, y, xg, jr); #c1(DMD) c2(NP_004011) c3(dystrophin Dp140c isoform) c4( )c5(142794, 142795, 142796, 142797, 142798, 142799, 142800, 142801, 142802, 142803, 142804, 142805, 142806, 142807, 142808, 142809, 142810, 142811, 142812, 142813, 142814, 142815, 142816, 142817, 142818, 142819, 142820, 142821, 142822, 142823, 142824, 142825, 142826, 142827, 142828, 142829, 142830, 142831, 142832, 142833, 142834, 142835, 142836, 142837, 142838, 142839, 142840, 142841, 142842, 142843, 142844, 142845, 142846, 142847, 142848, 142849, 142850, 142851, 142852, 142853, 142854, 142855, 142856, 142857, 142858, 142859, 142860, 142861, 142862, 142863, 142864, 142865, 142866, 142867, 142868, 142869, 142870, 142871, 142872, 142873, 142874, 142875, 142876, 142877, 142878, 142879) c6( )c7(T, ea, agU, bgs, afl, HU, bgl, aE, aDk, il, fm, ari, bgm, bgw, K, amA, hm, are, lW, uY, bp, pl, eY, Kc, aeB, bgu, mx, U, cj, nw, mk, m, lS, bgi, ayw, dD, Ol, aFc, Ri, Bm, n, aj, jA, nA, cd, afR, b, no, cc, pN, adX, Ak, afV, arc, bl, he, on, bgn, Hz, lb, Ad, mo, jT, fq, cA, adZ, dB, oh, rx, aiS, arg, cB, bgv, bL, cz, arw, j, bgq, eA, J, bgk, hW, Wn, kf, cy, aWs, bK, zp, yj, Wq, bA, bgp, VC, kl, aqZ, Qr, oV, Y, lo, rR, c, aqu, km, arf, ao, aof, hS, arh, bgj, eq, Rk, yt, sO, Ae, vJ, oj, Ps, bgr, fb, anR, xz, xs, hp, Bu, bz, dr, bgt, dR, Bv, yE, eB, bgo, ll, gZ, ol, i, y, xg, jr); #c1(DMD) c2(XP_006724531) c3(dystrophin isoform X1) c4(296030) c5( )c6( )c7(T, ea, agU, bgs, afl, HU, bgl, aE, aDk, il, fm, ari, bgm, bgw, K, amA, hm, are, lW, uY, bp, pl, eY, Kc, aeB, bgu, mx, U, cj, nw, mk, m, lS, bgi, ayw, dD, Ol, aFc, Ri, Bm, n, aj, jA, nA, cd, afR, b, no, cc, pN, adX, Ak, afV, arc, bl, he, on, bgn, Hz, lb, Ad, mo, jT, fq, cA, adZ, dB, oh, rx, aiS, arg, cB, bgv, bL, cz, arw, j, bgq, eA, J, bgk, hW, Wn, kf, cy, aWs, bK, zp, yj, Wq, bA, bgp, VC, kl, aqZ, Qr, oV, Y, lo, rR, c, aqu, km, arf, ao, aof, hS, arh, bgj, eq, Rk, yt, sO, Ae, vJ, oj, Ps, bgr, fb, anR, xz, xs, hp, Bu, bz, dr, bgt, dR, Bv, yE, eB, bgo, ll, gZ, oQ, i, y, xg, jr); #c1(DMD) c2(XP_006724533) c3(dystrophin isoform X3) c4(296031, 296032) c5(296033, 296034, 296035, 296036, 296037, 296038, 296039, 296040) c6( )c7(T, ea, agU, bgs, afl, HU, bgl, aE, aDk, il, fm, ari, bgm, bgw, K, amA, hm, are, lW, uY, bp, pl, eY, Kc, aeB, bgu, mx, U, cj, nw, mk, m, lS, bgi, ayw, dD, Ol, aFc, Ri, Bm, n, aj, jA, nA, cd, afR, b, no, cc, pN, adX, Ak, afV, arc, bl, he, on, bgn, Hz, lb, Ad, mo, jT, fq, cA, adZ, dB, oh, rx, aiS, arg, cB, bgv, bL, cz, arw, j, bgq, eA, J, bgk, hW, Wn, kf, cy, aWs, bK, zp, yj, Wq, bA, bgp, VC, kl, aqZ, Qr, oV, Y, lo, rR, c, aqu, km, arf, ao, aof, hS, arh, bgj, eq, Rk, yt, sO, Ae, vJ, oj, Ps, bgr, fb, anR, xz, xs, hp, Bu, bz, dr, bgt, dR, Bv, yE, eB, bgo, ll, gZ, ol, i, y, xg, jr); #c1(DMD) c2(XP_006724535) c3(dystrophin isoform X6) c4(296041) c5( ) c6( )c7(T, ea, agU, bgs, afl, HU, bgl, aE, aDk, il, fm, ari, bgm, bgw, K, amA, hm, are, lW, uY, bp, pl, eY, Kc, aeB, bgu, mx, U, cj, nw, mk, m, lS, bgi, ayw, dD, Ol, aFc, Ri, Bm, n, aj, jA, nA, cd, afR, b, no, cc, pN, adX, Ak, afV, arc, bl, he, on, bgn, Hz, lb, Ad, mo, jT, fq, cA, adZ, dB, oh, rx, aiS, arg, cB, bgv, bL, cz, arw, j, bgq, eA, J, bgk, hW, Wn, kf, cy, aWs, bK, zp, yj, Wq, bA, bgp, VC, kl, aqZ, Or, oV, Y, lo, rR, c, aqU, km, arf, ao, aof, hS, arh, bgj, eq, Rk, yt, s, Ae, vJ, oj, Ps, bgr, fb, anR, xz, xs, hp, Bu, bz, dr, bgt, dR, Bv, yE, eB, bgo, ll, gZ, oQ, i, y, xg, jr); #c1(DMD) c2(XP_006724536) c3(dystrophin isoform X7) c4( )c5(296042, 296043, 296044, 296045, 296046, 296047, 296048, 296049) c6( ) c7(T, ea, agU, bgs, afl, HU, bgl, aE, aDk, il, fm, ari, bgm, bgw, K, amA, hm, are, lW, uY, bp, pl, eY, Kc, aeB, bgu, mx, U, cj, nw, mk, m, lS, bgi, ayw, dD, Ol, aFc, Ri, Bm, n, aj, jA, nA, cd, afR, b, no, cc, pN, adX, Ak, afV, arc, bl, he, on, bgn, Hz, lb, Ad, mo, jT, fq, cA, adZ, dB, oh, rx, aiS, arg, cB, bgv, bL, cz, arw, j, bgq, eA, J, bgk, hW, Wn, kf, cy, aWs, bK, zp, yj, Wq, bA, bgp, VC, kl, aqZ, Ar, oV, Y, lo, rR, c, aqu, km, arf, ao, aof, hS, arh, bgj, eq, Rk, yt, sO, Ae, vJ, oj, Ps, bgr, fb, anR, xz, xs, hp, Bu, bz, dr, bgt, dR, Bv, yE, eB, bgo, ll, gZ, ol, i, y, xg, jr); #c1(DMD) c2(XP_006724537) c3(dystrophin isoform X8) c4(296050) c5(296051, 296052, 296053, 296054, 296055, 296056, 296057, 296058) c6( )c7(T, ea, agU, bgs, afl, HU, bgl, aE, aDk, il, fm, ari, bgm, bgw, K, amA, hm, are, lW, uY, bp, pl, eY, Kc, aeB, bgu, mx, U, cj, nw, mk, m, lS, bgi, ayw, dD, Ol, aFc, Ri, Bm, n, aj, jA, nA, cd, afR, b, no, cc, pN, adX, Ak, afV, arc, bl, he, on, bgn, Hz, lb, Ad, mo, jT, fq, cA, adZ, dB, oh, rx, aiS, arg, cB, bgv, bL, cz arw, j, bgq, eA, J, bgk, hW, Wn, kf, cy, aWs, bK, zp, yj, Wq, bA, bgp, VC, kl, aqZ, Ar, oV, Y, lo, rR, c, aqu, km, arf, ao, aof, hS, arh, bgj, eq, Rk, yt, sO, Ae, vJ, oj, Ps, bgr, fb, anR, xz, xs, hp, Bu, bz, dr, bgt, dR, Bv, yE, eB, bgo, ll, gZ, ol, i, y, xg, jr); #c1(DMD) c2(XP_006724538) c3(dystrophin isoform X9) c4(296059) c5(296060, 296061, 296062, 296063, 296064, 296065, 296066, 296067) c6( )c7(T, ea, agU, bgs, afl, HU, bgl, aE, aDk, il, fm, ari, bgm, bgw, K, amA, hm, are, lW, uY, bp, pl, eY, Kc, aeB, bgu, mx, U, cj, nw, mk, m, lS, bgi, ayw, dD, Ol, aFc, Ri, Bm, n, aj, jA, nA, cd, afR, b, no, cc, pN, adX, Ak, afV, arc, bl, he, on, bgn, Hz, lb, Ad, mo, jT, fq, cA, adZ, dB, oh, rx, aiS, arg, cB, bgv, bL, cz, arw, j, bgq, eA, J, bgk, hW, Wn, kf, cy, aWs, bK, zp, yj, Wq, bA, bgp, VC, kl, aqZ, Ur, oV, Y, lo, rR, c, aqu, km, arf, ao, aof, hS, arh, bgj, eq, Rk, yt, sO, Ae, vJ, oj, Ps, bgr, fb, anR, xz, xs, hp, Bu, bz, dr, bgt, dR, Bv, yE, eB, bgo, ll, gZ, ol, i, y, xg, jr); #c1(DMD) c2(XP_011543771) c3(dystrophin isoform X11) c4( )c5(342802, 342803, 342804, 342805, 342806, 342807, 342808, 342809, 342810, 342811, 342812, 342813, 342814, 342815, 342816, 342817, 342818, 342819, 342820, 342821, 342822, 342823, 342824, 342825, 342826, 342827, 342828, 342829, 342830, 342831, 342832, 342833, 342834, 342835, 342836, 342837, 342838, 342839, 342840, 342841, 342842, 342843, 342844, 342845, 342846, 342847, 342848, 342849, 342850, 342851, 342852, 342853, 342854, 342855, 342856, 342857, 342858, 342859, 342860, 342861, 342862, 342863, 342864, 342865, 342866, 342867, 342868, 342869, 342870, 342871, 342872, 342873, 342874, 342875, 342876, 342877, 342878, 342879, 342880, 342881, 342882, 342883, 342884, 342885, 342886, 342887) c6( )c7(T, ea, agU, bgs, afl, HU, bgl, aE, aDk, il, fm, ari, bgm, bgw, K, amA, hm, are, lW, uY, bp, pl, eY, Kc, aeB, bgu, mx, U, cj, nw, mk, m, lS, bgi, ayw, dD, Ol, aFc, Ri, Bm, n, aj, jA, nA, cd, afR, b, no, cc, pN, adX, Ak, afV, arc, bl, he, on, bgn, Hz, lb, Ad, mo, jT, fq, cA, adZ, dB, oh, rx, aiS, arg, cB, bgv, bL, cz, arw, j, bgq, eA, J, bgk, hW, Wn, kf, cy, aWs, bK, zp, yj, Wq, bA, bgp, VC, kl, aqZ, @r, oV, Y, lo, rR, c, aqu, km, arf, ao, aof, hS, arh, bgj, eq, Rk, yt, sO, Ae, vJ, oj, Ps, bgr, fb, anR, xz, xs, hp, Bu, bz, dr, bgt, dR, Bv, yE, eB, bgo, ll, gZ, ol, i, y, xg, jr); #c1(DMGDH) c2(NP_037523) c3(dimethylglycine dehydrogenase, mitochondrial precursor) c4(176005) c5(176006, 176007, 176008, 176009, 176010, 176011) c6( )c7(Na, mO, oe, bgx, Nc, j); #c1(DMGDH) c2(XP_006714660) c3(dimethylglycine dehydrogenase, mitochondrial isoform X1) c4(287951, 287952, 287953, 287954) c5(287955, 287956, 287957, 287958, 287959, 287960, 287961, 287962, 287963, 287964, 287965, 287966, 287967, 287968, 287969, 287970, 287971, 287972, 287973, 287974, 287975, 287976, 287977, 287978) c6( )c7(Na, mO, oe, bgx, Nc, j); #c1(DMKN) c2(NP_001030593) c3(dermokine isoform 1 precursor) c4(31055) c5(31056, 31057, 31058, 31059, 31060, 31061, 31062) c6(31063) c7(afr, ff, r, q, lQ, en, X); #c1(DMKN) c2(NP_001119528) c3(dermakine isoform 3 precursor) c4(44105) c5(44106, 44107, 44108, 44109, 44110, 44111, 44112, 44113) c6(44114, 44115) c7(afr, ff, r, q, lQ, en, X); #c1(DMKN) c2(NP_001119529) c3(dermokine isoform 5 precursor) c4(44116, 44117) c5( )c6( )c7(afr, ff, r, q, lQ, en, X); #c1(DMKN) c2(NP_001119530) c3(dermokine isoform 4 precursor) c4(44118) c5( )c6( )c7(afr, ff, r, q, lQ, en, X); #c1(DMKN) c2(NP_00119531) c3(dermokine isoform 6) c4(44119) c5(44120, 44121, 44122, 44123, 44124, 44125, 44126, 44127, 44128, 44129, 44130, 44131, 44132, 44133, 44134, 44135, 44136) c6(44137, 44138, 44139, 44140) c7(afr, ff, r, q, lQ, en, X); #c1(DMKN) c2(NP_001177276) c3(dermokine isoform 8 precursor) c4(72569) c5(72570, 72571, 72572, 72573, 72574, 72575, 72576, 72577, 72578) c6(72579, 72580) c7(afr, ff, r, q, lQ, en, X); #c1(DMKN) c2(NP_001177277) c3(dermokine isoform 9 precursor) c4(72581) c5( )c6( )c7(afr, ff, r, q, lQ, en, X); #c1(DMKN) c2(NP_001177278) c3(dermokine isoform 10 precursor) c4(72582, 72583) c5(72584, 72585, 72586, 72587, 72588, 72589, 72590, 72591, 72592, 72593, 72594, 72595, 72596, 72597, 72598, 72599, 72600, 72601, 72602, 72603, 72604, 72605, 72606, 72607, 72608, 72609, 72610, 72611, 72612, 72613, 72614, 72615, 72616, 72617, 72618, 72619, 72620, 72621, 72622, 72623, 72624, 72625, 72626, 72627, 72628, 72629, 72630) c6(72631, 72632, 72633, 72634, 72635, 72636, 72637, 72638, 72639, 72640, 72641, 72642) c7(afr, ff, r, q, lQ, en, X); #c1(DMKN) c2(NP_201574) c3(dermokine isoform 2 precursor) c4(228286) c5( )c6( )c7(afr, ff, r, q, lQ, en, X); #c1(DMKN) c2(XP_011525806) c3(dermokine isoform X9) c4( )c5(320204) c6( )c7(afr, ff, r, q, lQ, en, X); #c1(DMKN) c2(XP_011525814) c3(dermokine isoform X17) c4( )c5(320205) c6( )c7(afr, ff, r, q, lQ, en, X); #c1(DMP1) c2(NP_001073380) c3(dentin matrix acidic phosphoprotein 1 isoform 2 precursor) c4(35992) c5(35993, 35994, 35995, 35996, 35997) c6( )c7(AB, oe, jh, Ay, amC, ci, av, amB, aT, hP, aW, ff, en, g, AA, bz, oU, QG, eM, ki, X, bfl, awa, ars); #c1(DMP1) c2(NP_004398) c3(dentin matrix acidic phosphoprotein 1 isoform 1 precursor) c4(146104) c5(146105, 146106, 146107, 146108, 146109) c6( )c7(AB, De, jh, Ay, amC, ci, av, amB, aT, hP, aW, ff, en, g, AA, bz, oU, QG, eM, ki, X, bfl, awa, ars); #c1(DMP1) c2(XP_011530007) c3(dentin matrix acidic phosphoprotein 1 isoform X1) c4( )c5(325813) c6( ) c7(AB, oe, jh, Ay, amC, ci, av, amB, aT, hP, aW, ff, en, g, AA, bz, oU, QG, eM, ki, X, bfl, awa, ars); #c1(DMP1) c2(XP_011530008) c3(dentin matrix acidic phosphoprotein 1 isoform X2) c4( )c5(325814) c6( )c7(AB, oe, jh, Ay, amC, ci, av, amB, aT, hP, aW, ff, en, g, AA, bz, oU, QG, eM, ki, X, bfl, awa, ars); #c1(DMPK) c2(NP_001075029) c3(myotonin-protein kinase isoform 3) c4(37194, 37195) c5(37196, 37197, 37198, 37199, 37200, 37201, 37202, 37203, 37204, 37205, 37206, 37207, 37208) c6( )c7(dD, Kk, aj, eq, aPl, nA, UX, UE, bgz, alD, oj, Ak, vH, vc, bl, K, dF, mD, xz, xs, aPm, hp, aac, dr, dR, rR, bgy, KB, aer, awk, lM, fy, gq); #c1(DMPK) c2(NP_001075031) c3(myotonin-protein kinase isoform 4) c4(37209) c5(37210, 37211, 37212, 37213, 37214, 37215, 37216) c6( )c7(dD, Kk, aj, eq, aPl, nA, UX, UE, bgz, alD, oj, Ak, vH, vc, bl, K, dF, mD, xz, xs, aPm, hp, aac, dr, dR, rR, bgy, KB, aer, awk, lM, fy, gq); #c1(DMPK) c2(NP_001075032) c3(myotonin-protein kinase isoform 1) c4(37217, 37218) c5(37219, 37220, 37221, 37222, 37223, 37224, 37225, 37226, 37227, 37228, 37229, 37230, 37231) c6( ) c7(dD, Kk, aj, eq, aPl, nA, UX, UE, bgz, alD, oj, Ak, vH, vc, bl, K, dF, mD, xz, xs, aPm, hp, aac, dr, dR, rR, bgy, kB, aer, awK, lM, fy, gq); #c1(DMPK) c2(NP_001275693) c3(myotonin-protein kinase isoform 5) c4(107079) c5(107080, 107081, 107082, 107083, 107084, 107085, 107086) c6( )c7(dD, Kk, aj, eq, aPl, nA, UX, UE, bgz, alD, oj, Ak, vH, vc, bl, K, dF, mD, xz, xs, aPm, hp, aac, dr, dR, rR, bgy, kB, aer, awk, lM, fy, gq); #c1(DMPK) c2(NP_001275695) c3(myotonin-protein kinase isoform 7) c4(107087) c5(107088, 107089, 107090, 107091, 107092, 107093, 107094) c6( )c7(dD, Kk, aj, eq, aPl, nA, UX, UE, bgz, alD, oj, Ak, vH, vc, bl, K, dF, mD, xz, xs, aPm, hp, aac, dr, dR, rR, bgy, kB, aer, awk, lM, fy, gq); #c1(DMPK) c2(NP_004400) c3(myotonin-protein kinase isoform 2) c4(146125) c5(146126, 146127, 146128, 146129, 146130, 146131, 146132) c6( )c7(dD, Kk, aj, eq, aPl, nA, UX, UE, bgz, alD, oj, Ak, vH, vc, bl, K, dF, mD, xz, xs, aPm, hp, aac, dr, dR, rR, bgy, kB, aer, awk, lM, fy, gq); #c1(DMRT1) c2(NP_068770) c3(doublesex- and mab-3-related transcription factor 1) c4(210308) c5(210309, 210310, 210311, 210312, 210313, 210314, 210315, 210316, 210317, 210318, 210319, 210320, 210321) c6( )c7(wu, e, ci, Rc, ber, ad, yw, vJ, oW, yu, cy, UD, rO, wm, eu, bgA, vT, Lf, bu, Ld, BU, ac, aNv, Qu, wO, Lb, aAE, aNt); #c1(DMRT1) c2(XP_006716795) c3(doublesex- and mab-3-related transcription factor 1 isoform X3) c4(290488) c5( )c6( )c7(wu, e, ci, Rc, ber, ad, yw, vJ, oW, yu, cy, UD, rO, wm, eu, bgA, vT, Lf, bu, Ld, BU, ac, aNv, Qu, wO, Lb, aAE, aNt); #c1(DMRT2) c2(XP_006716781) c3(doublesex- and mab-3-related transcription factor 2 isoform X3) c4( )c5(290451, 290452) c6( )c7(it); #c1(DMRT2) c2(XP_011515995) c3(doublesex- and mab-3-related transcription factor 2 isoform XI) c4(309/77) c5(309178, 309179, 309180, 309181, 309182, 309183, 309184, 309185, 309186, 309187, 309188, 309189, 309190, 309191, 309192, 309193) c6( )c7(it); #c1(DMRT2) c2(XP_011515998) c3(doublesex- and mab-3-related transcription factor 2 isoform X2) c4(309194) c5(309195, 309196, 309197, 309198, 309199, 309200, 309201, 309202, 309203, 309204, 309205, 309206) c6( ) c7(it); #c1(DMRT3) c2(NP_067063) c3(doublesex- and mab-3-related transcription factor 3) c4(208795, 208796) c5(208797, 208798, 208799, 208800, 208801, 208802, 208803) c6( )c7(gP, vT, Qu, Pl, B, aR); #c1(DMRT3) c2(XP_011516299) c3(doublesex- and mab-3-related transcription factor 3 isoform X1) c4( )c5(309504, 309505, 309506, 309507, 309508, 309509) c6( )c7(gP, vT, Qu, PI, B, aR); #c1(DMRTA1) c2(NP_071443) c3(doublesex- and mab-3-related transcription factor A1) c4(211694) c5(211695, 211696, 211697, 211698, 211699, 211700, 211701) c6( )c7(Ea, oe, baZ, fq, aA); #c1(DMTF1) c2(NP_001135798) c3(cyclin-D-binding Myb-like transcription factor 1 isoform b) c4( )c5(53287) c6( )c7(av, amB, QG, AB, eM, aW, jh, aT, Ay, amC, bK, ci, bfl, AA, bz, awa, ars, oU); #c1(DMTF1) c2(XP_011515039) c3(cyclin-D-binding Myb-like transcription factor 1 isoform X1) c4(307809, 307810, 307811, 307812) c5(307813, 307814, 307815, 307816, 307817, 307818, 307819, 307820, 307821, 307822, 307823, 307824, 307825, 307826, 307827, 307828, 307829, 307830, 307831, 307832, 307833, 307834, 307835, 307836, 307837, 307838, 307839) c6( )c7(av, amB, QG, AB, eM, aW, jh, aT, Ay, amC, bK, ci, bfl, AA, bz, awa, ars, oU); #c1(DMTF1) c2(XP_011515041) c3(cyclin-D-binding Myb-like transcription factor 1 isoform X2) c4(307840) c5(307841, 307842, 307843, 307844, 307845, 307846, 307847, 307848, 307849) c6( )c7(av, amB, QG, AB, eM, aW, jh, aT, Ay, amC, bK, ci, bfl, AA, bz, awa, ars, ou); #c1(DMTF1) c2(XP_011515042) c3(cyclin-D-binding Myb-like transcription factor 1 isoform X3) c4(307850, 307851) c5( )c6( )c7(av, amB, QG, AB, eM, aW, jh, aT, Ay, amC, bK, ci, bfl, AA, bz, awa, ars, oU); #c1(DMTF1) c2(XP_011515043) c3(cyclin-D-binding Myb-like transcription factor 1 isoform X4) c4(307852, 307853) c5(307854, 307855, 307856, 307857, 307858, 307859, 307860, 307861, 307862, 307863, 307864, 307865, 307866) c6( )c7(av, amB, QG, AB, eM, aW, jh, aT, Ay, amC, bK, ci, bfl, AA, bz, awa, ars, ou); #c1(DMTF1) c2(XP_011515044) c3(cyclin-D-binding Myb-like transcription factor 1 isoform X5) c4(307867, 307868, 307869, 307870, 307871, 307872, 307873, 307874) c5(307875, 307876, 307877, 307878, 307879, 307880, 307881, 307882, 307883, 307884, 307885, 307886, 307887, 307888, 307889, 307890, 307891, 307892, 307893, 307894, 307895, 307896, 307897, 307898, 307899, 307900, 307901, 307902, 307903, 307904, 307905, 307906, 307907, 307908, 307909, 307910, 307911, 307912, 307913, 307914, 307915, 307916, 307917, 307918, 307919, 307920, 307921, 307922, 307923, 307924, 307925, 307926, 307927, 307928, 307929, 307930, 307931, 307932, 307933, 307934, 307935, 307936, 307937, 307938, 307939, 307940, 307941, 307942, 307943, 307944, 307945) c6( )c7(av, amB, QG, AB, eM, aW, jh, aT, Ay, amC, bK, ci, bfl, AA, bz, awa, ars, oU); #c1(DMTN) c2(NP_001107611) c3(dematin isoform 3) c4(42917) c5(42918, 42919, 42920, 42921, 42922, 42923, 42924, 42925) c6( )c7(S, oW); #c1(DMTN) c2(NP_001289745) c3(dematin isoform 2) c4(114374) c5(114375, 114376, 114377, 114378, 114379, 114380, 114381, 114382, 114383, 114384, 114385, 114386, 114387, 114388) c6( )c7(S, oW); #c1(DMTN) c2(NP_001289746) c3(dematin isoform 4) c4(114389) c5(114390, 114391, 114392, 114393, 114394, 114395) c6( )c7(S, oW); #c1(DMTN) c2(XP_005273489) c3(dematin isoform X1) c4(284611, 284612) c5(284613, 284614, 284615, 284616, 284617, 284618, 284619, 284620, 284621, 284622, 284623, 284624, 284625, 284626, 284627, 284628, 284629, 284630, 284631) c6( )c7(S, oW); #c1(DMTN) c2(XP_011542740) c3(dematin isoform X4) c4( )c5(341316, 341317, 341318, 341319, 341320) c6( )c7(S, oW); #c1(DMWD) c2(NP_004934) c3(dystrophia myotonica WD repeat-containing protein) c4(151234) c5(151235, 151236, 151237, 151238, 151239, 151240) c6( )c7(gU, ND, dR); #c1(DMXL1) c2(NP_001277250) c3(dmX-like protein 1 isoform 1) c4(109301) c5(109302, 109303, 109304, 109305, 109306, 109307) c6( )c7(dw, d, eA); #c1(DMXL1) c2(NP_005500) c3(dmX-like protein 1 isoform 2) c4(156851) c5(156852, 156853, 156854, 156855, 156856, 156857) c6( )c7(dw, d, eA); #c1(DMXL1) c2(XP_005271967) c3(dmX-like protein 1 isoform X2) c4(283653) c5(283654, 283655, 283656, 283657, 283658, 283659) c6( )c7(dw, d, eA); #c1(DMXL2) c2(NP_00167587) c3(dmX-like protein 2 isoform 1) c4(69330) c5(69331, 69332, 69333, 69334, 69335, 69336) c6( )c7(bM, ac, aw, e, j, eu); #c1(DMXL2) c2(NP_001167588) c3(dmX-like protein 2 isoform 3) c4(69337) c5(69338, 69339, 69340, 69341, 69342, 69343) c6( )c7(bM, ac, aw, e, j, eu); #c1(DMXL2) c2(NP_056078) c3(dmX-like protein 2 isoform 2) c4(185560) c5(185561, 185562, 185563, 185564, 185565, 185566) c6( )c7(bM, ac, aw, e, j, eu); #c1(DMXL2) c2(XP_005254313) c3(dmX-like protein 2 isoform X2) c4(273141) c5(273142, 273143, 273144, 273145, 273146, 273147, 273148, 273149) c6( )c7(bM, ac, aw, e, j, eu); #c1(DNA2) c2(NP_001073918) c3(DNA replication ATP-dependent helicase/nuclease DNA2) c4(36520) c5(36521, 36522, 36523, 36524, 36525, 36526, 36527, 36528, 36529, 36530) c6( )c7(ck, Mb, os, WE, oj, h, aNG, bgB, dL, K, oZ, avx); #c1(DNA2) c2(XP_006717743) c3(DNA replication ATP-dependent helicase/nuclease DNA2 isoform X1) c4(291305, 291306) c5(291307, 291308, 291309, 291310, 291311, 291312, 291313) c6( )c7(ck, Mb, os, WE, oj, h, aNG, bgB, dL, K, oZ, avx); #c1(DNA2) c2(XP_011537721) c3(DNA replication ATP-dependent helicase/nuclease DNA2 isoform X5) c4( )c5(335279) c6( )c7(ck, Mb, os, WE, oj, h, aNG, bgB, dL, K, oZ, avx); #c1(DNAAF1) c2(NP_848547) c3(dynein assembly factor 1, axonemal) c4(253813, 253814) c5(253815, 253816, 253817, 253818, 253819, 253820, 253821, 253822, 253823, 253824, 253825, 253826) c6( )c7(Pf, bgC, wB, aBZ, Nj, MG); #c1(DNAAF1) c2(XP_011521162) c3(dynein assembly factor 1, axonemal isoform X11) c4( ) c5(316108) c6( )c7(Pf, bgC, wB, aBZ, Nj, MG); #c1(DNAAF2) c2(NP_001077377) c3(protein kintoun isoform 2) c4(37781) c5(37782, 37783, 37784, 37785, 37786, 37787) c6( )c7(bgD); #c1(DNAAF2) c2(NP_060609) c3(protein kintoun isoform 1) c4(196827) c5(196828, 196829, 196830, 196831, 196832, 196833) c6( )c7(bgD); #c1(DNAAF3) c2(NP_001243643) c3(dynein assembly factor 3, axonemal isoform 1) c4(91282) c5(91283, 91284, 91285, 91286, 91287, 91288, 91289, 91290, 91291, 91292) c6( )c7(bN, T, aaF, dt, ul, Pf, bgE, hL, fd, ame, bgF, cl, Vq, cm, BV, xg, af, LS, DF, MG); #c1(DNAAF3) c2(NP_001243644) c3(dynein assembly factor 3, axonemal isoform 3) c4(91293) c5(91294, 91295, 91296, 91297, 91298) c6( )c7(bN, T, aaF, dt, ul, Pf, bgE, hL, fd, ame, bgF, cl, Vq, cm, BV, xg, af, LS, DF, MG); #c1(DNAAF3) c2(NP_001243645) c3(dynein assembly factor 3, axonemal isoform 4) c4(91299) c5(91300, 91301, 91302, 91303, 91304, 91305, 91306) c6( )c7(bN, T, aaF, dt, ul, Pf, bgE, hl, fd, ame, bgF, cl, Vq, cm, BV, xg, af, LS, DF, MG); #c1(DNAAF3) c2(NP_849159) c3(dynein assembly factor 3, axonemal isoform 2) c4(254326) c5(254327, 254328, 254329, 254330, 254331, 254332, 254333, 254334, 254335, 254336) c6( )c7(bN, T, aaF, dt, ul, Pf, bgE, hL, fd, ame, bgF, cl, Vq, cm, BV, xg, af, LS, DF, MG); #c1(DNAAF5) c2(NP_060272) c3(dynein assembly factor 5, axonemal) c4(195146, 195147, 195148) c5(195149, 195150, 195151, 195152, 195153, 195154, 195155, 195156, 195157, 195158, 195159, 195160, 195161, 195162, 195163, 195164, 195165, 195166) c6( )c7(Pf, MG, bgG); #c1(DNAH1) c2(NP_056327) c3(dynein heavy chain 1, axonemal) c4(187086) c5(187087, 187088, 187089, 187090, 187091, 187092, 187093, 187094, 187095, 187096, 187097, 187098, 187099) c6( )c7(MG); #c1(DNAH1) c2(XP_011531879) c3(dynein heavy chain 1, axonemal isoform X1) c4(327931) c5( )c6( )c7(MG); #c1(DNAH11) c2(NP_001264044) c3(dynein heavy chain 11, axonemal) c4(97441) c5(97442, 97443, 97444, 97445, 97446, 97447) c6( )c7(bgl, ZE, BV, Pf, bgH, hb, fd, afq, j, aXL, fw, af, MG); #c1(DNAH12) c2(NP_001278590) c3(dynein heavy chain 12, axonemal isoform 3) c4(109869) c5(109870, 109871, 109872, 109873, 109874, 109875, 109876, 109877, 109878, 109879, 109880, 109881, 109882, 109883, 109884, 109885, 109886, 109887, 109888, 109889, 109890) c6( )c7(ac, ad); #c1(DNAH12) c2(NP_940966) c3(dynein heavy chain 12, axonemal isoform 2) c4(260202) c5(260203, 260204, 260205, 260206, 260207, 260208) c6( )c7(ac, ad); #c1(DNAH12) c2(XP_011531772) c3(dynein heavy chain 12, axonemal isoform X5) c4(327856) c5( )c6( )c7(ac, ad); #c1(DNAH12) c2(XP_011531773) c3(dynein heavy chain 12, axonemal isoform X6) c4(327857) c5(327858, 327859) c6( )c7(ac, ad); #c1(DNAH17) c2(NP_775899) c3(dynein heavy chain (7, axonemal) c4(250611, 250612) c5(250613, 250614, 250615, 250616, 250617, 250618) c6( ) c7(ul, MG); #c1(DNAH17) c2(XP_011523718) c3(dynein heavy chain 17, axonemal isoform X1) c4( )c5(318177, 318178, 318179, 318180, 318181, 318182) c6( )c7(ul, MG); #c1(DNAH17) c2(XP_011523720) c3(dynein heavy chain 17, axonemal isoform X3) c4( )c5(318183) c6( )c7(ul, MG); #c1(DNAH2) c2(NP_001290199) c3(dynein heavy chain 2, axonemal isoform 2) c4(115124) c5(115125, 115126, 115127, 115128, 115129, 115130, 115131, 115132, 115133, 115134, 115135, 115136, 115137, 115138, 115139, 115140) c6( )c7(A); #c1(DNAH2) c2(NP_065928) c3(dynein heavy chain 2, axonemal isoform 1) c4(206293, 206294) c5(206295, 206296, 206297, 206298, 206299, 206300, 206301, 206302, 206303, 206304, 206305, 206306, 206307, 206308, 206309, 206310) c6( )c7(A); #c1(DNAH3) c2(NP_060009) c3(dynein heavy chain 3, axonemal) c4(193614, 193615, 193616) c5(193617, 193618, 193619, 193620, 193621, 193622, 193623, 193624, 193625, 193626, 193627, 193628, 193629, 193630, 193631, 193632, 193633) c6( )c7(ul, MG); #c1(DNAH3) c2(XP_006721125) c3(dynein heavy chain 3, axonemal isoform X2) c4(293861) c5(293862, 293863, 293864, 293865, 293866, 293867) c6( )c7(ul, MG); #c1(DNAH5) c2(NP_001360) c3(dynein heavy chain 5, axonemal) c4(117289) c5(117290, 117291, 117292, 117293, 117294, 117295) c6( )c7(Mg, bM, ul, bgJ, ZE, aBU, Pf, aYJ, oe, fd, ame, bgF, Vq, LS, DF, MG); #c1(DNAH6) c2(NP_001361) c3(dynein heavy chain 6, axonemal) c4(117296) c5(117297, 117298, 117299, 117300, 117301, 117302, 117303, 117304, 117305, 117306, 117307) c6( )c7(fq); #c1(DNAH6) c2(XP_011530955) c3(dynein heavy chain 6, axonemal isoform X3) c4(326923) c5(326924, 326925, 326926, 326927, 326928, 326929) c6( )c7(fq); #c1(DNAH7) c2(NP_06/720) c3(dynein heavy chain 7, axonemal) c4(200377, 200378) c5(200379, 200380, 200381, 200382, 200383, 200384, 200385, 200386, 200387, 200388, 200389, 200390) c6( )c7(cm, MG, Ea); #c1(DNAH7) c2(XP_011509793) c3(dynein heavy chain 7, axonemal isoform X5) c4(300348, 300349) c5( )c6( )c7(cm, MG, Ea); #c1(DNAH7) c2(XP_011509797) c3(dynein heavy chain 7, axonemal isoform X9) c4( ) c5(300350, 300351, 300352, 300353, 300354, 300355, 300356, 300357, 300358, 300359, 300360, 300361) c6( )c7(cm, MG, Ea); #c1(DNAH8) c2(NP_001193856) c3(dynein heavy chain 8, axonemal) c4(82750, 82751, 82752) c5(82753, 82754, 82755, 82756, 82757, 82758, 82759, 82760, 82761, 82762, 82763, 82764, 82765, 82766, 82767, 82768, 82769, 82770) c6( )c7(gh, T, lP, aPR, zs, e, akz, bB, fd, ur, ci, akg, oJ, bgM, aR, AW, aE, JD, dV, aU, K, bc, azh, Ok, DA, aiV, ajT, JI, alf, aW, az, hl, kt, uY, La, pl, hJ, eY, bb, akx, eM, wE, TG, Kr, mx, U, cj, alc, uo, wB, aL, mk, bi, eg, iu, NK, gU, sw, aw, ht, akA, aj, bd, cd, aky, cc, mh, ep, jy, aYf, AX, A, aJ, Ad, ail, aq, mo, dw, aYo, ajS, W, cA, bP, eo, ajW, bgO, aCD, bD, alo, al, B, sZ, lB, bL, bgL, mr, fG, oe, yk, jx, rf, aA, cR, mS, cz, v, j, sl, bgR, akG, aYD, q, J, eA, akE, Mg, uT, lg, aT, Wk, cy, aiW, bgN, bK, an, ae, af, aju, wr, s, Y, r, wl, ayr, cu, en, f, kC, nC, ik, akq, rY, ck, Qi, akF, cl, ac, aki, ig, bal, mG, au, hr, wR, mf, aan, fD, bt, VB, ao, eP, pY, ob, bgP, bgQ, jN, jh, dG, fu, aoz, Pu, ad, av, aV, arV, oj, bgK, vc, lQ, cb, alk, mu, lR, xz, ff, eZ, aF, la, akh, iV, HI, bz, dr, qa, dR, LB, Uj, Xp, bE, akm, uR, ll, gZ, aks, X); #c1(DNAH8) c2(XP_011512621) c3(dynein heavy chain 8, axonemal isoform X2) c4(303586) c5(303587, 303588, 303589, 303590, 303591, 303592) c6( )c7(gh, T, lP, aPR, zs, e, akz, bB, fd, ur, ci, akg, oJ, bgM, aR, AW, aE, JD, dV, aU, K, bc, azh, Ok, DA, aiV, ajT, JI, alf, aW, az, hl, kt, uY, La, pl, hJ, eY, bb, akx, eM, wE, TG, Kr, mx, U, cj, alc, uo, wB, aL, mk, bi, eg, iu, NK, gU, sw, aw, ht, akA, aj, bd, cd, aky, cc, mh, ep, jy, aYf, AX, A, aJ, Ad, ail, aq, mo, dw, aYo, ajS, W, cA, bP, eo, ajW, bgO, aCD, bD, alo, al, B, sZ, lB, bL, bgL, mr, fG, oe, yk, jx, rf, aA, cR, mS, cz, v, j, sl, bgR, akG, aYD, q, J, eA, akE, Mg, uT, lg, aT, Wk, cy, aiW, bgN, bK, an, ae, af, ajU, wr, s, Y, r, wl, ayr, cu, en, f, kC, nC, ik, akq, rY, ck, Qi, akF, cl, ac, aki, ig, bal, mG, au, hr, wR, mf, aan, fD, bt, VB, ao, eP, pY, ob, bgP, bgQ, jN, jh, dG, fu, aoz, Pu, ad, av, aV, arV, oj, bgK, vc, lQ, cb, alk, mu, lR, xz, ff, eZ, aF, la, akh, iV, HI, bz, dr, qa, dR, LB, Uj, Xp, bE, akm, uR, ll, gZ, aks, X); #c1(DNAH9) c2(NP_001363) c3(dynein heavy chain 9, axonemal isoform 2) c4(117308, 117309, 117310, 117311) c5(117312, 117313, 117314, 117315, 117316, 117317, 117318, 117319, 117320, 117321, 117322, 117323, 117324, 117325, 117326, 117327, 117328, 117329, 117330, 117331, 117332, 117333, 117334, 117335) c6( )c7(Pf, Bd, dY, Be, MG); #c1(DNAH9) c2(XP_011522005) c3(dynein heavy chain 9, axonemal isoform X1) c4(316695) c5(316696, 316697, 316698, 316699, 316700, 316701) c6( )c7(Pf, Bd, dY, Be, MG); #c1(DNA11) c2(NP_036276) c3(dynein intermediate chain 1, axonemal isoform 1) c4(172823, 172824) c5(172825, 172826, 172827, 172828, 172829, 172830, 172831, (72832) c6( )c7(ul, LS, ZE, Pf, il, iW, fd, MG, bw, ame, bgF, Vq, zZ, BV, br, DF, afu); #c1(DNA11) c2(XP_006716821) c3(dynein intermediate chain 1, axonemal isoform X5) c4( )c5(290509, 290510, 290511, 290512, 290513, 290514, 290515, 290516, 290517, 290518, 290519) c6( ) c7(ul, LS, ZE, Pf, il, iW, fd, MG, bw, ame, bgF, Vq, zZ, BV, br, DF, afu); #c1(DNA11) c2(XP_011516148) c3(dynein intermediate chain 1, axonemal isoform X1) c4(309383, 309384) c5( )c6( )c7(ul, LS, ZE, Pf, il, iW, fd, MG, bw, ame, bgF, Vq, zZ, BV, br, DF, afu); #c1(DNA11) c2(XP_011516149) c3(dynein intermediate chain 1, axonemal isoform X2) c4(309385) c5( )c6( )c7(ul, LS, ZE, Pf, il, iW, fd, MG, bw, ame, bgF, Vq, zZ, BV, br, DF, afu); #c1(DNA11) c2(XP_011516152) c3(dynein intermediate chain 1, axonemal isoform X6) c4( )c5(309386, 309387, 309388, 309389, 309390, 309391, 309392, 309393, 309394, 309395, 309396, 309397, 309398, 309399, 309400) c6( ) c7(ul, LS, ZE, Pf, il, iW, fd, MG, bw, ame, bgF, Vq, zZ, BV, br, DF, afu); #c1(DNA12) c2(NP_001166281) c3(dynein intermediate chain 2, axonemal isoform 2) c4(68536) c5(68537, 68538, 68539, 68540, 68541, 68542) c6( )c7(cU, lo, Pf, MG, bgS); #c1(DNA12) c2(NP_075462) c3(dynein intermediate chain 2, axonemal isoform 1) c4(213888, 213889) c5(213890, 213891, 213892, 213893, 213894, 213895, 213896, 213897, 213898, 213899, 213900, 213901, 213902, 213903, 213904, 213905) c6( )c7(cu, lo, Pf, MG, bgS); #c1(DNAJA1) c2(NP_001530) c3 (dnaJ homolog subfamily A member 1) c4(118640, 118641, 118642) c5(118643, 118644, 118645, 118646, 118647, 118648, (18649, 118650) c6( )c7(aC, app, avn, Kj, eZ); #c1(DNAJA2) c2(NP_005871) c3(dnaJ homolog subfamily A member 2) c4(160039) c5(160040, 160041, 160042, 160043, 160044, 160045) c6( )c7(t, y, K, hl, m); #c1(DNAJA3) c2(NP_001128582) c3(dnaJ homolog subfamily A member 3, mitochondrial isoform 2) c4(50336) c5(50337, 50338, 50339, 50340, 50341, 50342) c6( )c7(bF, T, xA, mx, aT, t, eA); #c1(DNAJA3) c2(NP_001273445) c3(dnaJ homolog subfamily A member 3, mitochondrial isoform 3) c4(104917) c5(104918, 104919, 104920, 104921, 104922, 104923) c6( )c7(bF, T, xA, mx, aT, t, eA); #c1(DNAJA3) c2(NP_005138) c3(dnaJ homolog subfamily A member 3, mitochondrial isoform 1) c4(153323) c5(153324, 153325, 153326, 153327, 153328, 153329) c6( )c7(bF, T, xA, mx, aT, t, eA); #c1(DNAJB1) c2(NP_001287843) c3 (dnaJ homolog subfamily B member 1 isoform 2) c4( )c5(112994, 112995, 112996, 112997, 112998) c6( )c7(ID, bM, fR, aZ, He, bcE, fr, eW, hX, dV, bK, K, oi, ly, aq, bu, iV, lx, eM, Kv, mx, U, km, Kj, B); #c1(DNAJB1) c2(NP_006136) c3(dnaJ homolog subfamily B member 1 isoform 1) c4(162303, 162304) c5(162305, 162306, 162307, 162308, 162309, 162310, 162311) c6( )c7(ID, bM, fR, aZ, He, bcE, fr, eW, hX, dV, bK, K, oi, ly, aq, bu, iV, lx, eM, Kv, mx, U, km, Kj, B); #c1(DNAJB11) c2(NP_057390) c3(dnaJ homolog subfamily B member Il precursor) c4(190520) c5(190521, 190522, 190523, 190524, 190525, 190526, 190527, 190528, 190529, 190530) c6( )c7(t, m); #c1(DNAJB13) c2(NP_705842) c3(dnaJ homolog subfamily B member 13) c4(246344) c5(246345, 246346, 246347, 246348, 246349, 246350) c6( )c7(cF); #c1(DNAJB13) c2(XP_005274041) c3(dnaJ homolog subfamily B member 13 isoform X3) c4(284911) c5( )c6( ) c7(cF); #c1(DNAJB13) c2(XP_011543314) c3(dnaJ homolog subfamily B member 13 isoform X4) c4( )c5(342096) c6( )c7(cF); #c1(DNAJB13) c2(XP_011543317) c3(dnaJ homolog subfamily B member 13 isoform X7) c4( )c5(342097, 342098, 342099, 342100, 342101, 342102, 342103, 342104, 342105) c6( )c7(cF); #c1(DNAJB2) c2(NP_001034639) c3(dnaJ homolog subfamily B member 2 isoform a) c4(31958) c5(31959, 31960, 31961, 31962, 31963, 31964, 31965, 31966, 31967, 31968, 31969, 31970, 31971, 31972, 31973, 31974, 31975, 31976, 31977, 31978, 31979, 31980, 31981, 31982) c6( )c7(rR, ql, c, bT, aem, oj, U, uY, bgT, Kj, fu, bV); #c1(DNAJB2) c2(NP_006727) c3(dnaJ homolog subfamily B member 2 isoform b) c4(167474, 167475, 167476, 167477) c5(167478, 167479, 167480, 167481, 167482, 167483) c6( )c7(rR, ql, c, bT, aem, oj, U, uY, bgT, Kj, fu, bV); #c1(DNAJB5) c2(NP_001128476) c3 (dnaJ homolog subfamily B member 5 isoform 2) c4(50036, 50037, 50038, 50039, 50040) c5(50041, 50042, 50043, 50044, 50045, 50046, 50047, 50048, 50049, 50050, 50051, 50052, 50053, 50054) c6( )c7(IV); #c1(DNAJB5) c2(NP_001128477) c3(dnaJ homolog subfamily B member 5 isoform 1) c4(50055, 50056, 50057) c5(50058, 50059, 50060, 50061, 50062, 50063, 50064, 50065, 50066, 50067, 50068, 50069, 50070, 50071, 50072, 50073, 50074, 50075, 50076, 50077, 50078, 50079) c6( )c7(IV); #c1(DNAJB5) c2(NP_036398) c3(dnaJ homolog subfamily B member 5 isoform 3) c4( )c5(173608, 173609, 173610, 173611, 173612, 173613, 173614, 173615, 173616, 173617, 173618, 173619, 173620, 173621, 173622, 173623, 173624, 173625, 173626, 173627, 173628) c6( )c7(IV); #c1(DNAJB6) c2(NP_005485) c3(dnaJ homolog subfamily B member 6 isoform b) c4(156641, 156642, 156643, 156644) c5(156645, 156646, 156647, 156648, 156649, 156650, 156651, 156652, 156653, 156654, 156655, 156656, 156657, 156658, 156659, 156660, 156661, 156662, 156663, 156664, 156665, 156666, 156667, 156668, 156669, 156670, 156671, 156672, 156673, 156674, 156675, 156676, 156677, 156678, 156679, 156680, 156681, 156682, 156683, 156684, 156685, 156686, 156687, 156688, 156689, 156690, 156691, 156692, 156693, 156694, 156695, 156696, 156697, 156698, 156699, 156700, 156701, 156702, 156703, 156704) c6( )c7(Y, oj, aT, km, bgU, K, m, t, y); #c1(DNAJB6) c2(NP_490647) c3(dnaJ homolog subfamily B member 6 isoform a) c4(231031, 231032) c5(231033, 231034, 231035, 231036, 231037, 231038) c6( )c7(Y, oj, aT, km, bgU, K, m, t, y); #c1(DNAJB6) c2(XP_006715886) c3(dnaJ homolog subfamily B member 6 isoform X2) c4(289883, 289884, 289885) c5( ) c6( )c7(Y, oj, aT, km, bgU, K, m, t, y); #c1(DNAJB6) c2(XP_011514006) c3(dnaJ homolog subfamily B member 6 isoform X1) c4(306412, 306413) c5( )c6( )c7(Y, oj, aT, km, bgU, K, m, t, y); #c1(DNAJB7) c2(NP_660157) c3(dnaJ homolog subfamily B member 7) c4(240422) c5(240423, 240424, 240425, 240426, 240427, 240428) c6( )c7(T, dA, bE, lH, ff, ik, en, qN, rA); #c1(DNAJB8) c2(NP_699161) c3(dnaJ homolog subfamily B member 8) c4(245749) c5(245750, 245751, 245752, 245753, 245754, 245755, 245756, 245757, 245758, 245759) c6( )c7(bE, r, bB, fr, bu, q); #c1(DNAJB9) c2(NP_036460) c3(dnaJ homolog subfamily B member 9 precursor) c4(174033) c5(174034, 174035, 174036, 174037, 174038, 174039) c6( )c7(hb, wL, K); #c1(DNAJC1) c2(NP_071760) c3(dnaJ homolog subfamily C member 1 precursor) c4(211979, 211980, 211981) c5(211982, 211983, 211984, 211985, 211986, 211987, 211988) c6( )c7(Y, ac, an); #c1(DNAJC1) c2(XP_011517917) c3(dnaJ homolog subfamily C member 1 isoform X2) c4( )c5(311862, 311863, 311864, 311865, 311866, 311867, 311868, 311869, 311870, 311871, 311872) c6( )c7(Y, ac, an); #c1(DNAJC10) c2(NP_001258510) c3(dnaJ homolog subfamily C member 10 isoform 2 precursor) c4(96083) c5(96084, 96085, 96086, 96087, 96088, 96089, 96090, 96091, 96092, 96093, 96094, 96095) c6( )c7(T, U, ey, K, cm, B, ad); #c1(DNAJC10) c2(NP_061854) c3(dnaJ homolog subfamily C member 10 isoform 1 precursor) c4(200806, 200807, 200808) c5(200809, 200810, 200811, 200812, 200813, 200814, 200815, 200816, 200817, 200818, 200819, 200820) c6( )c7(T, U, ey, K, cm, B, ad); #c1(DNAJC12) c2(NP_068572) c3(dnaJ homolog subfamily C member 12 isoform a) c4(209441) c5(209442, 209443, 209444, 209445, 209446, 209447) c6( )c7(af); #c1(DNAJC12) c2(NP_957714) c3(dnaJ homolog subfamily C member 12 isoform b) c4(262016) c5(262017, 262018, 262019, 262020, 262021, 262022) c6( )c7(af); #c1(DNAJC13) c2(NP_056083) c3(dnaJ homolog subfamily C member 13) c4(185589) c5(185590, 185591, 185592, 185593, 185594, 185595, 185596, 185597, 185598, 185599, 185600, 185601, 185602) c6( )c7(aW, j, an, aT); #c1(DNAJC13) c2(XP_005247302) c3(dnaJ homolog subfamily C member 13 isoform X1) c4(267761) c5(267762, 267763, 267764, 267765) c6( )c7(aW, j, an, aT); #c1(DNAJC14) c2(NP_115740) c3(dnaJ homolog subfamily C member 14) c4(223759, 223760, 223761, 223762, 223763) c5(223764, 223765, 223766, 223767, 223768, 223769, 223770, 223771, 223772, 223773, 223774, 223775, 223776, 223777, 223778, 223779, 223780, 223781, 223782, 223783, 223784, 223785, 223786, 223787, 223788, 223789, 223790, 223791, 223792, 223793, 223794, 223795, 223796, 223797, 223798, 223799, 223800, 223801) c6( )c7(t, y, K, m); #c1(DNAJC15) c2(NP_037370) c3(dnaJ homolog subfamily C member 15) c4(175085) c5(175086, 175087, 175088, 175089, 175090, 175091) c6( )c7(iC, jf, T, dB, aW, Y, aT, aA, aF, m, t); #c1(DNAJC18) c2(NP_689899) c3(dnaJ homolog subfamily C member 18) c4(244297, 244298, 244299, 244300) c5(244301, 244302, 244303, 244304, 244305, 244306, 244307, 244308, 244309, 244310, 244311, 244312, 244313, 244314, 244315, 244316, 244317, 244318, 244319, 244320, 244321, 244322, 244323, 244324) c6( )c7(at, bw, dL); #c1 (DNAJC19) c2(NP_660304) c3(mitochondrial import inner membrane translocase subunit TIM14 isoform 1) c4(240808) c5(240809, 240810, 240811, 240812, 240813, 240814) c6( )c7(gZ, cc, dr, mx, bgV); #c1(DNAJC2) c2(NP_001123359) c3(dnaJ homolog subfamily C member 2 isoform 2) c4(47013) c5(47014, 47015, 47016, 47017, 47018, 47019, 47020, 47021, 47022, 47023, 47024, 47025, 47026, 47027, 47028, 47029, 47030, 47031) c6( )c7(ck, T, il, ff, en, bt); #c1(DNAJC2) c2(NP_055192) c3(dnaJ homolog subfamily C member 2 isoform 1) c4(179680, 179681) c5( )c6( )c7(ck, T, iU, ff, en, bt); #c1(DNAJC27) c2(NP_057628) c3(dnaJ homolog subfamily C member 27 isoform 1) c4(191948, 191949) c5(191950, 191951, 191952, 191953, 191954, 191955, 191956, 191957, 191958, 191959, 191960, 191961) c6( )c7(v, gX); #c1(DNAJC28) c2(NP_060303) c3(dnaJ homolog subfamily C member 28) c4(195280) c5(195281, 195282, 195283, 195284, 195285, 195286, 195287, 195288, 195289, 195290, 195291, 195292, 195293, 195294, 195295, 195296, 195297, 195298, 195299, 195300) c6( ) c7(Y); #c1(DNAJC3) c2(NP_006251) c3(dnaJ homolog subfamily C member 3 precursor) c4(163293) c5(163294, 163295, 163296, 163297, 163298, 163299) c6( )c7(B, ad, r); #c1(DNAJC5) c2(NP_079495) c3(dnaJ homolog subfamily C member 5) c4(218378, 218379) c5(218380, 218381, 218382, 218383, 218384, 218385, 218386, 218387, 218388, 218389, 218390, 218391, 218392) c6( )c7(T, apM, hq, aPQ, eM, aPR, aW, jZ, Gb, aPS, aT, U, aPT, ci, nA, bgW, aR, rY, alX); #c1(DNAJC7) c2(NP_001138238) c3(dnaJ homolog subfamily C member 7 isoform 2) c4(56127) c5( )c6( )c7(h); #c1(DNAJC7) c2(NP_003306) c3(dnaJ homolog subfamily C member 7 isoform 1) c4(137322) c5(137323, 137324, 137325, 137326, 137327, 137328, 137329, 137330, 137331, 137332, 137333, 137334, 137335, 137336) c6( )c7(h); #c1(DNAJC7) c2(XP_011523468) c3(dnaJ homolog subfamily C member 7 isoform X2) c4( )c5(318004, 318005) c6( )c7(h); #c1(DNAL1) c2(NP_113615) c3(dynein light chain 1, axonemal isoform 1) c4(221735, 221736, 221737, 221738, 221739, 221740, 221741) c5(221742, 221743, 221744, 221745, 221746, 221747, 221748, 221749, 221750, 221751, 221752) c6( )c7(bgX, MG); #c1(DNAL1) c2(XP_011535506) c3(dynein light chain 1, axonemal isoform X1) c4( )c5(332560, 332561, 332562, 332563, 332564, 332565, 332566, 332567, 332568, 332569, 332570, 332571, 332572, 332573, 332574, 332575, 332576, 332577, 332578, 332579, 332580, 332581, 332582, 332583, 332584, 332585, 332586, 332587, 332588, 332589, 332590, 332591, 332592, 332593, 332594) c6( )c7(bgX, MG); #c1(DNASE1) c2(XP_011520696) c3(deoxyribonuclease-1 isoform X1) c4(315617) c5(315618, 315619, 315620, 315621, 315622, 315623, 315624, 315625, 315626, 315627, 315628, 315629, 315630, 315631, 315632, 315633, 315634, 315635, 315636, 315637, 315638, 315639, 315640, 315641, 315642, 315643, 315644, 315645, 315646, 315647, 315648, 315649, 315650, 315651, 315652, 315653, 315654, 315655, 315656, 315657, 315658, 315659, 315660, 315661, 315662, 315663, 315664, 315665, 315666, 315667, 315668, 315669, 315670, 315671, 315672, 315673, 315674, 315675, 315676, 315677, 315678, 315679, 315680, 315681, 315682, 315683, 315684, 315685, 315686, 315687, 315688, 315689, 315690, 315691, 315692, 315693, 315694, 315695, 315696, 315697, 315698, 315699, 315700, 315701, 315702, 315703, 315704, 315705, 315706, 315707, 315708, 315709, 315710, 315711, 315712, 315713, 315714, 315715, 315716, 315717, 315718) c6(315719, 315720, 315721, 315722, 315723) c7(T, eR, qb, aw, jh, cz, bB, hz, uE, cd, q, ad, rc, er, aE, aT, hD, h, K, wt, hK, ae, hm, yF, aa, aW, hl, eZ, aq, aVD, aF, ho, Vk, cm, aFA, ck, avS, bE, hc, fl, pw, y, B, ao); #c1(DNASE1) c2(XP_011520697) c3(deoxyribonuclease-1 isoform X3) c4(315724) c5(315725) c6( )c7(T, eR, qb, aw, jh, cz, bB, hz, uE, cd, q, ad, rc, er, aE, aT, hD, h, K, wt, hK, ae, hm, yF, aa, aW, hl, eZ, aq, aVD, aF, ho, Vk, cm, aFA, ck, avS, bE, hc, fl, pw, y, B, ao); #c1(DNASE1) c2(XP_011520700) c3(deoxyribonuclease-1 isoform X6) c4( )c5(315726, 315727, 315728, 315729, 315730, 315731, 315732) c6(315733, 315734) c7(T, eR, qb, aw, jh, cz, bB, hz, uE, cd, q, ad, rc, er, aE, aT, hD, h, K, wt, hK, ae, hm, yF, aa, aW, hl, eZ, aq, aVD, aF, ho, Vk, cm, aFA, ck, avS, bE, hc, fl, pw, y, B, ao); #c1(DNASEIL2) c2(NP_001288609) c3(deoxyribonuclease-1-like 2 precursor) c4( )c5(113814, 113815, 113816, 113817, 113818, 113819, 113820, 113821, 113822, 113823, 113824, 113825, 113826, 113827, 113828, 113829, 113830, 113831, 113832) c6(113833, 113834, 113835, 113836, 113837, 113838) c7(ad); #c1(DNASEIL2) c2(XP_011520701) c3(deoxyribonuclease-1-like 2 isoform X1) c4(315735, 315736) c5(315737, 315738, 315739) c6( )c7(ad); #c1(DNASEIL3) c2(NP_001243489) c3(deoxyribonuclease gamma isoform 2 precursor) c4(90930) c5(90931, 90932, 90933, 90934, 90935, 90936) c6( )c7(vU, ajH, bgY, av, bgZ, bha, eH, eZ, jh, gl, ti, bS, du, eF, rz, eA, y, bN, qc); #c1(DNASEIL3) c2(NP_004935) c3(deoxyribonuclease gamma isoform 1 precursor) c4(151241, 151242, 151243) c5(151244, 151245, 151246, 151247, 151248, 151249, 151250, 151251, 151252, 151253, 151254, 151255, 151256, 151257, 151258, 151259, 151260, 151261, 151262, 151263, 151264, 151265) c6( )c7(vU, ajH, bgY, av, bgZ, bha, eH, eZ, jh, gl, ti, bS, du, eF, rz, eA, y, bN, qc); #c1 (DNASE2) c2(NP_001366) c3(deoxyribonuclease-2-alpha precursor) c4(117336, 117337) c5(117338, 117339, 117340, 117341, 117342, 117343, 117344, 117345, 117346, (17347, 117348, 117349) c6( )c7(oO, eZ, cy, dy, fl, y); #c1(DNASE2B) c2(XP_011540180) c3(deoxyribonuclease-2-beta isoform X1) c4( )c5(338563, 338564, 338565, 338566, 338567) c6( )c7(F); #c1(DND1) c2(NP_919225) c3(dead end protein homolog 1) c4(258254) c5(258255, 258256, 258257, 258258, 258259, 258260) c6( )c7(Lb, vT, bu); #c1(DNER) c2(NP_620711) c3(delta and Notch-like epidermal growth factor-related receptor precursor) c4(238097, 238098) c5(238099, 238100, 238101, 238102, 238103, 238104, 238105, 238106, 238107, 238108, 238109, 238110) c6( )c7(bL, Kd, T, dD, ck, x, jf, ea, fd, h, eq, k, cm, bt, cd); #c1(DNHD1) c2(NP_653267) c3(dynein heavy chain domain-containing protein 1 isoform 1) c4(239412, 239413, 239414) c5(239415, 239416, 239417, 239418, 239419, 239420, 239421, 239422, 239423, 239424, 239425, 239426, 239427, 239428, 239429, 239430, 239431) c6( )c7(fr); #c1(DNHD1) c2(NP_775860) c3(dynein heavy chain domain-containing protein 1 isoform 2) c4( )c5(250500, 250501, 250502, 250503, 250504) c6( )c7(fr); #c1(DNLZ) c2(NP_001074318) c3(DNL-type zinc finger protein) c4(37089) c5(37090, 37091, 37092, 37093, 37094, 37095) c6( )c7(T, if, aj, Gn, qN, eW, J, hX, jz, h, CW, apn, aq, Ej, iR, cm, ov, il, rH, im, rA, B); #c1(DNM1) c2(NP_001005336) c3(dynamin-1 isoform 2) c4(21388, 21389) c5(21390, 21391, 21392, 21393, 21394, 21395) c6( ) c7(VU, Qi, Cq, aTK, hb, XY, ha, fc, lm, cP, bU, af, aR); #c1(DNM1) c2(NP_001275666) c3(dynamin-I isoform 3) c4(106994) c5(106995, 106996, 106997, 106998, 106999, 107000, 107001, 107002, 107003, 107004, 107005, 107006, 107007, 107008, 107009, 107010) c6( ) c7(vU, Qi, Cq, aTK, hb, XY, ha, fc, lm, cP, bU, af, aR); #c1(DNM1) c2(NP_001275668) c3(dynamin-1 isoform 4) c4(107011) c5(107012, 107013, 107014, 107015, 107016, 107017) c6( )c7(vU, Qi, Cq, aTK, hb, XY, ha, fc, lm, cP, bU, af, aR); #c1(DNM1) c2(NP_004399) c3(dynamin-1 isoform 1) c4(146110, 146111) c5(146112, 146113, 146114, 146115, 146116, 146117, 146118, 146119, 146120, 146121, 146122, 146123, 146124) c6( )c7(vU, Qi, Cq, aTK, hb, XY, ha, fc, lm, cP, bU, af, aR); #c1(DNM1) c2(XP_005251820) c3(dynamin-I isoform X4) c4(271200) c5( )c6( )c7(vU, di, Cq, aTK, hb, XY, ha, fc, lm, cP, bU, af, aR); #c1(DNM1L) c2(NP_001265392) c3(dynamin-I-like protein isoform 4) c4(98484) c5(98485, 98486, 98487, 98488, 98489, 98490) c6( )c7(ID, T, dG, j, ad, Ae, aT, cy, K, an, ae, af, ly, aW, ff, en, g, QK, px, bhb, lx, wg, c, SE, eM, U, awK, lC, NN); #c1(DNM1L) c2(NP_001265393) c3(dynamin-1-like protein isoform 5) c4(98491) c5(98492, 98493, 98494, 98495, 98496, 98497) c6( )c7(ID, T, dG, j, ad, Ae, aT, cy, K, an, ae, af, ly, aW, ff, en, g, QK, px, bhb, lx, wg, c, SE, eM, U, awk, lC, NN); #c1(DNM1L) c2(NP_001265394) c3(dynamin-1-like protein isoform 6) c4(98498) c5(98499, 98500, 98501, 98502, 98503, 98504) c6( )c7(ID, T, dG, j, ad, Ae, aT, cy, K, an, ae, af, ly, aW, ff, en, g, QK, px, bhb, lx, wg, c, SE, eM, U, awk, lC, NN); #c1(DNM1L) c2(NP_001265395) c3 (dynamin-1-like protein isoform 7) c4(98505) c5(98506, 98507, 98508, 98509, 98510, 98511) c6( )c7(ID, T, dG, j, ad, Ae, aT, cy, K, an, ae, af, ly, aW, ff, en, g, QK, px, bhb, lx, wg, c, SE, eM, U, awk, lC, NN); #c1(DNM1L) c2(NP_005681) c3(dynamin-1-like protein isoform 3) c4(158498, 158499) c5(158500, 158501, 158502, 158503, 158504, 158505, 158506, 158507, 158508, 158509, 158510, 158511) c6( )c7(ID, T, dG, j, ad, Ae, aT, cy, K, an, ae, af, ly, aW, ff, en, g, QK, px, bhb, lx, wg, c, SE, eM, U, awK, lC, NN); #c1(DNM1L) c2(NP_036192) c3(dynamin-1-like protein isoform ( )c4(172323) c5(172324, 172325, 172326, 172327, 172328, 172329) c6( )c7(ID, T, dG, j, ad, Ae, aT, cy, K, an, ae, af, ly, aW, ff, en, g, QK, px, bhb, lx, wg, c, SE, eM, U, awk, lC, NN); #c1(DNM1L) c2(NP_036193) c3(dynamin-I-like protein isoform 2) c4(172330) c5(172331, 172332, 172333, 172334, 172335, 172336) c6( )c7(ID, T, dG, j, ad, Ae, aT, cy, K, an, ae, af, ly, aW, ff, en, g, QK, px, bhb, lx, wg, c, SE, eM, U, awK, lC, NN); #c1(DNM2) c2(NP_001005360) c3(dynamin-2 isoform 1) c4(21455) c5(21456, 21457, 21458, 21459, 21460, 21461) c6( )c7(gV, T, aC, fR, Pt, aio, UU, arS, amp, Ri, aA, n, cz, aj, j, aTe, ci, fu, bX, bB, qN, UX, fK, J, aR, cd, ad, hX, eA, hd, no, aE, oj, aT, Ak, h, bhf, K, bl, bU, bV, ae, bhe, af, mu, jf, bhh, d, bhg, s, hG, aW, bT, xz, bhi, ayn, Ad, akB, dq, aF, bhd, BG, as, iV, dr, qa, zW, c, bE, mx, U, bhc, bk, aS, IC, B, UV, cn); #c1(DNM2) c2(NP_001005361) c3(dynamin-2 isoform 2) c4(21462, 21463) c5(21464, 21465, 21466, 21467, 21468, 21469, 21470, 21471, 21472, 21473, 21474, 21475, 21476) c6( )c7(gV, T, aC, fR, Pt, aio, UU, arS, amp, Ri, aA, n, cz, aj, j, aTe, ci, fu, bX, bB, qN, UX, fK, J, aR, cd, ad, hX, eA, hd, no, aE, oj, aT, Ak, h, bhf, K, bl, bU, bV, ae, bhe, af, mu, jf, bhh, d, bhg, s, hG, aW, bT, xz, bhi, ayn, Ad, akB, dq, aF, bhd, BG, as, iV, dr, qa, zW, c, bE, mx, U, bhc, bk, aS, lC, B, UV, cn); #c1(DNM2) c2(NP_001005362) c3(dynamin-2 isoform 4) c4(21477, 21478, 21479) c5(21480, 21481, 21482, 21483, 21484, 21485, 21486, 21487, 21488, 21489, 21490, 21491, 21492, 21493, 21494, 21495, 21496) c6( ) c7(gV, T, aC, fR, Pt, aio, UU, arS, amp, Ri, aA, n, cz, aj, j, aTe, ci, fu, bX, bB, qN, UX, fK, J, aR, cd, ad, hX, eA, hd, no, aE, oj, aT, Ak, h, bhf, K, bl, bU, bV, ae, bhe, af, mu, jf, bhh, d, bhg, s, hG, aW, bT, xz, bhi, ayn, Ad, akB, dq, aF, bhd, BG, as, iV, dr, qa, zW, c, bE, mx, U, bhc, bk, aS, lC, B, UV, cn); #c1(DNM2) c2(NP_004936) c3(dynamin-2 isoform 3) c4(151266) c5(151267, 151268, 151269, 151270, 151271, 151272) c6( )c7(gV, T, aC, fR, Pt, aio, UU, arS, amp, Ri, aA, n, cz, aj, j, aTe, ci, fu, bX, bB, qN, UX, fK, J, aR, cd, ad, hX, eA, hd, no, aE, oj, aT, Ak, h, bhf, K, bl, bU, bV, ae, bhe, af, mu, jf, bhh, d, bhg, s, hG, aW, bT, xz, bhi, ayn, Ad, akB, dq, af, bhd, BG, as, iV, dr, qa, zW, c, bE, mx, U, bhc, bk, aS, lC, B, UV, cn); #c1(DNM3) c2(NP_001129599) c3(dynamin-3 isoform b) c4(51976, 51977) c5(51978, 51979, 51980, 51981, 51982, 51983) c6( )c7(xR, T, au, afW, el); #c1(DNM3) c2(NP_001265181) c3(dynamin-3 isoform c) c4( )c5(97927, 97928, 97929, 97930, 97931) c6( )c7(xR, T, au, afW, el); #c1(DNM3) c2(NP_056384) c3(dynamin-3 isoform a) c4(187370) c5(187371, 187372, 187373, 187374, 187375, 187376, 187377) c6( )c7(xR, T, au, afW, el); #c1(DNM3) c2(XP_005245136) c3(dynamin-3 isoform X1) c4(266316) c5(266317) c6( )c7(xR, T, au, afW, el); #c1(DNM3) c2(XP_005245137) c3(dynamin-3 isoform X2) c4(266318) c5(266319, 266320, 266321, 266322, 266323, 266324) c6( ) c7(xR, T, au, afW, ell); #c1(DNM3) c2(XP_011507703) c3(dynamin-3 isoform X5) c4(297709) c5(297710, 297711, 297712, 297713, 297714, 297715) c6( )c7(xR, T, au, afW, ell); #c1(DNMBP) c2(NP_056036) c3(dynamin-binding protein) c4(185236, 185237) c5(185238, 185239, 185240, 185241, 185242, 185243, 185244, 185245, 185246, 185247, 185248, 185249, 185250, 185251) c6( )c7(af); #c1(DNMT1) c2(NP_001124295) c3(DNA (cytosine-5)-methyltransferase 1 isoform a) c4(48515, 48516) c5(48517, 48518, 48519, 48520, 48521, 48522) c6( )c7(ng, bM, T, if, al, bO, bB, lR, ci, Lw, ip, aE, K, eL, yo, zQ, iE, aW, WE, kt, g, eE, bw, bb, eM, lp, vA, U, cj, uJ, m, gU, wu, iB, n, fr, qN, fK, cd, RD, jz, dL, ZN, he, fe, aq, kp, bhk, mo, dw, op, hq, bP, cA, eD, hL, hR, FL, bD, it, B, cn, bL, fG, ard, aA, il, AO, q, eA, cF, aT, vH, bK, Fg, ae, jf, ak, ND, nO, iU, Y, Lf, r, lo, en, cl, bu, t, iK, pb, ck, c, pZ, bhl, bt, bhj, vi, id, Pt, aFa, ha, jh, bS, hF, mC, ad, hX, dP, av, ib, py, wm, nJ, d, qt, ff, gK, eZ, gl, aF, Fq, bz, LB, bF, gk, bE, y, X); #c1(DNMT1) c2(NP_001370) c3 (DNA (cytosine-5)-methyltransferase 1 isoform b) c4(117391) c5(117392, 117393, 117394, 117395, 117396, 117397) c6( )c7(ng, bM, T, if, al, bO, bB, lR, ci, Lw, ip, aE, K, eL, yo, zl, iE, aW, WE, kt, g, eE, bw, bb, eM, lp, vA, U, cj, uJ, m, gU, wu, iB, n, fr, qN, fK, cd, RD, jz, dL, ZN, he, fe, aq, kp, bhk, mo, dw, op, hq, bP, cA, eD, hL, hR, FL, bD, it, B, cn, bL, fG, arQ, aA, il, AO, q, eA, cF, aT, vH, bK, Fg, ae, jf, ak, ND, no, iU, Y, Lf, r, lo, en, cl, bu, t, iK, pb, ck, c, pZ, bhl, bt, bhj, vi, id, Pt, aFa, ha, jh, bS, hF, mC, ad, hX, dP, av, ib, py, wm, nJ, d, qt, ff, gK, eZ, gl, aF, Fq, bz, LB, bF, gk, bE, y, X); #c1(DNMT3A) c2(NP_715640) c3(DNA (cytosine-5)-methyltransferase 3A isoform b) c4(246706) c5(246707, 246708, 246709, 246710, 246711, 246712) c6( )c7(T, wi, id, if, iW, ha, ht, al, aA, fd, oY, jh, fr, ci, nA, qN, il, hF, q, mC, br, ad, dP, oB, hX, er, av, aE, x, KN, KH, aT, dL, ox, jj, hu, eL, d, zH, iU, iZ, aW, oV, ff, Az, eZ, r, gm, aF, Fq, en, t, bw, bz, Hv, op, yR, ck, bF, gk, bP, c, eM, lp, vA, k, m, fl, bD, bt, xb, B, vi); #c1 (DNMT3A) c2(NP_783328) c3(DNA (cytosine-5)-methyltransferase 3A isoform a) c4(251653, 251654) c5(251655, 251656, 251657, 251658, 251659, 251660, 251661, 251662, 251663, 251664, 251665, 251666, 251667, 251668, 251669, 251670) c6( )c7(T, wi, id, if, iW, ha, ht, al, aA, fd, oY, jh, fr, ci, nA, qN, il, hF, q, mC, br, ad, dP, oB, hX, er, av, aE, x, KN, KH, aT, dL, ox, jj, hu, eL, d, zH, il, iZ, aW, oV, ff, Az, eZ, r, gm, aF, Fq, en, t, bw, bz, Hv, op, yR, ck, bF, gk, bP, c, eM, lp, vA, k, m, fl, bD, bt, xb, B, vi); #c1(DNMT3A) c2(NP_783329) c3(DNA (cytosine-5)-methyltransferase 3A isoform c) c4(251671) c5(251672, 251673, 251674, 251675, 251676, 251677) c6( )c7(T, wi, id, if, iW, ha, ht, al, aA, fd, oY, jh, fr, ci, nA, qN, il, hF, q, mC, br, ad, dP, oB, hX, er, av, aE, x, KN, KH, aT, dL, ox, jj, hu, eL, d, zH, il, iZ, aW, oV, ff, Az, eZ, r, gm, aF, Fq, en, t, bw, bz, Hv, op, yR, ck, bF, gk, bP, c, eM, lp, vA, k, m, fl, bD, bt, xb, B, vi); #c1(DNMT3B) c2(NP_001193984) c3(DNA (cytosine-5)-methyltransferase 3B isoform 7) c4(83428, 83429) c5(83430, 83431, 83432, 83433, 83434) c6( )c7(T, FR, if, al, bO, fd, bB, ci, aE, u, hu, DY, bhm, aW, Az, g, cm, eM, cj, sv, uJ, m, gU, wu, sw, ht, afE, fr, nA, qN, zP, ft, jz, h, hB, ZN, he, Cw, AQ, aq, kp, hq, bP, cA, dB, eD, hL, rx, FL, bD, B, fM, aA, hy, pd, q, aT, ae, ne, jf, qw, hM, s, zH, iU, oV, Y, ev, r, lo, en, cl, t, f, iK, bt, ao, vi, id, jh, Hn, bS, mC, ad, dP, av, l, wm, BN, BO, ff, eZ, aF, jb, bz, dr, bF, alE, gk, bE, X, y, iu); #c1(DNMT3B) c2(NP_001193985) c3(DNA (cytosine-5)-methyltransferase 38 isoform 8) c4(83435) c5(83436, 83437, 83438, 83439, 83440, 83441) c6( )c7(T, fR, if, al, bO, fd, bB, ci, aE, u, hu, DY, bhm, aW, Az, g, cm, eM, cj, sv, uJ, m, gU, wu, sw, ht, afE, fr, nA, qN, zP, ft, jz, h, hB, ZN, he, Cw, AQ, aq, kp, hq, bP, cA, dB, eD, hL, rx, fL, bD, B, fM, aA, hy, pd, q, aT, ae, ne, jf, qw, hM, s, zH, il, oV, Y, ev, r, lo, en, c1, t, f, ik, bt, ao, vi, id, jh, Hn, bS, mC, ad, dP, av, l, wm, BN, BO, ff, eZ, aF, jb, bz, dr, bF, alE, gk, bE, X, y, iu); #c1(DNMT3B) c2(NP_008823) c3(DNA (cytosine-5)-methyltransferase 38 isoform 1) c4(168832) c5(168833, 168834, 168835, 168836, 168837, 168838, 168839) c6( ) c7(T, fR, if, al, bO, fd, bB, ci, aE, u, hu, DY, bhm, aW, Az, g, cm, eM, cj, sv, uJ, m, gU, wu, sw, ht, afE, fr, nA, qN, zP, ft, jz, h, hB, ZN, he, Cw, AQ, aq, kp, hq, bP, cA, dB, eD, hL, rx, fL, bD, B, fM, aA, hy, pd, q, aT, ae, ne, jf, qw, hM, s, zH, il, oV, Y, ev, r, lo, en, cl, t, f, ik, bt, ao, vi, id, jh, Hn, bS, mC, ad, dP, av, l, wm, BN, BO, ff, eZ, aF, jb, bz, dr, bF, alE, gk, bE, X, y, iu); #c1(DNMT3B) c2(NP_787044) c3(DNA (cytosine-5)-methyltransferase 38 isoform 2) c4(251989, 251990, 251991) c5( )c6( )c7(T, fR, if, al, bO, fd, bB, ci, aE, u, hu, DY, bhm, aW, Az, g, cm, eM, cj, sv, uJ, m, gU, wu, sw, ht, afE, fr, nA, qN, zP, ft, jz, h, hB, ZN, he, Cw, AQ, aq, kp, hq, bP, cA, dB, eD, hL, rx, fL, bD, B, fM, aA, hy, pd, q, aT, ae, ne, jf, qw, hM, s, zH, il, oV, Y, ev, r, lo, en, cl, t, f, ik, bt, ao, vi, id, jh, Hn, bS, mC, ad, dP, av, l, wm, BN, BO, ff, eZ, aF, jb, bz, dr, bF, alE, gk, bE, X, y, iu); #c1(DNMT3B) c2(NP_787045) c3(DNA (cytosine-5)-methyltransferase 38 isoform 3) c4(251992) c5( )c6( )c7(T, FR, if, al, bO, fd, bB, ci, aE, u, hu, DY, bhm, aW, Az, g, cm, eM, cj, sv, uJ, m, gU, wu, sw, ht, afE, fr, nA, qN, zP, ft, jz, h, hB, ZN, he, Cw, AQ, aq, kp, hq, bP, cA, dB, eD, hL, rx, fL, bD, B, fM, aA, hy, pd, q, aT, ae, ne, jf, qw, hM, s, zH, il, oV, Y, ev, r, lo, en, cl, t, f, ik, bt, ao, vi, id, jh, Hn, bS, mC, ad, dP, av, l, wm, BN, BO, ff, eZ, aF, jb, bz, dr, bF, alE, gk, bE, X, y, iu); #c1(DNMT3B) c2(NP_787046) c3(DNA (cytosine-5)-methyltransferase 3B isoform 6) c4(251993) c5(251994, 251995, 251996, 251997, 251998, 251999, 252000, 252001, 252002, 252003, 252004, 252005, 252006, 252007, 252008, 252009, 252010, 252011) c6( )c7(T, fR, if, al, bO, fd, bB, ci, aE, u, hu, DY, bhm, aW, Az, g, cm, eM, cj, sv, uJ, m, gU, wu, sw, ht, afE, fr, nA, qN, zP, ft, jz, h, hB, ZN, he, Cw, AQ, aq, kp, hq, bP, cA, dB, eD, hL, rx, fL, bD, B, fM, aA, hy, pd, q, aT, ae, ne, jf, qw, hM, s, zH, iU, oV, Y, ev, r, lo, en, cl, t, f, iK, bt, ao, vi, id, jh, Hn, bS, mC, ad, dP, av, l, wm, BN, BO, ff, eZ, aF, jb, bz, dr, bF, alE, gk, bE, X, y, iu); #c1(DNMT3B) c2(XP_011526955) c3(DNA (cytosine-5)-methyltransferase 3B isoform X1) c4( )c5(321245, 321246, 321247, 321248, 321249, 321250, 321251, 321252, 321253, 321254, 321255, 321256) c6( )c7(T, FR, if, al, bO, fd, bB, ci, aE, u, hu, DY, bhm, aW, Az, g, cm, eM, cj, sv, uJ, m, gU, wu, sw, ht, afE, fr, nA, qN, zP, ft, jz, h, hB, ZN, he, Cw, AQ, aq, kp, hq, bP, cA, dB, eD, hL, rx, fL, bD, B, fM, aA, hy, pd, q, aT, ae, ne, jf, qw, hM, s, zH, il, oV, Y, ev, r, lo, en, cl, t, f, iK, bt, ao, vi, id, jh, Hn, bS, mC, ad, dP, av, l, wm, BN, BO, ff, eZ, aF, jb, bz, dr, bF, alE, gk, bE, X, y, iu); #c1(DNMT3B) c2(XP_011526956) c3(DNA (cytosine-5)-methyltransferase 38 isoform X2) c4( )c5(321257, 321258, 321259, 321260, 321261, 321262) c6( )c7(T, fR, if, al, bO, fd, bB, ci, aE, u, hu, DY, bhm, aW, Az, g, cm, eM, cj, sv, uJ, m, gU, wu, sw, ht, afE, fr, nA, qN, zP, ft, jz, h, hB, ZN, he, Cw, AQ, aq, kp, hq, bP, cA, dB, eD, hL, rx, FL, bD, B, fM, aA, hy, pd, q, aT, ae, ne, jf, qw, hM, s, zH, il, oV, Y, ev, r, lo, en, cl, t, f, ik, bt, ao, vi, id, jh, Hn, bS, mC, ad, dP, av, l, wm, BN, BO, ff, eZ, aF, jb, bz, dr, bF, alE, gk, bE, X, y, iu); #c1(DNMT3L) c2(NP_787063) c3(DNA (cytosine-5)-methyltransferase 3-like isoform 2) c4(252054) c5(252055, 252056, 252057, 252058, 252059, 252060) c6( )c7(T, gU, wu, Fo, eD, ard, bt, vH, m, wm, bw, Px); #c1(DNMT3L) c2(XP_011527838) c3(DNA (cytosine-5)-methyltransferase 3-like isoform X1) c4(322771) c5(322772, 322773, 322774, 322775, 322776, 322777) c6( )c7(T, gU, wu, Fo, eD, ard, bt, vH, m, wm, bw, Px); #c1(DNPEP) c2(NP_036232) c3(aspartyl aminopeptidase) c4(172508, 172509) c5(172510, 172511, 172512, 172513, 172514, 172515, 172516, 172517, 172518, 172519, 172520, 172521) c6( )c7(T, fM, eP, ha, jh, aA, Bm, n, vm, eb, fd, ci, q, XX, hW, Vr, dL, eL, r, aF, apm, iK, bz, ck, BM, cj, hR); #c1(DNPEP) c2(XP_011509185) c3(aspartyl aminopeptidase isoform X1) c4(299801) c5( )c6( )c7(T, fM, eP, ha, jh, aA, Bm, n, vm, eb, fd, ci, q, XX, hW, Vr, dL, eL, r, aF, apm, ik, bz, ck, eM, cj, hR); #c1(DNPEP) c2(XP_011509189) c3(aspartyl aminopeptidase isoform X3) c4( )c5(299802, 299803, 299804, 299805, 299806, 299807) c6( )c7(T, fM, eP, ha, jh, aA, Bm, n, vm, eb, fd, ci, q, XX, hW, Vr, dL, eL, r, aF, apm, ik, bz, ck, eM, cj, hR); #c1(DNTT) c2(NP_001017520) c3(DNA nucleotidylexotransferase isoform 2) c4(26539) c5(26540, 26541, 26542, 26543, 26544, 26545) c6( )c7(bM, iH, acl, yY, cz, v, j, ad, Mg, Na, iX, HD, bK, K, rz, ae, ahE, A, yL, eZ, fe, ahl, bN, ck, ou, mx, yA, y, bt); #c1(DNTT) c2(NP_004079) c3(DNA nucleotidylexotransferase isoform 1) c4(143374) c5(143375, 143376, 143377, 143378, 143379, 143380) c6( )c7(bM, iH, acl, yY, cz, v, j, ad, Mg, Na, iX, hD, bK, K, rz, ae, ahE, A, yL, eZ, fe, ahl, bN, ck, ou, mx, yA, y, bt); #c1(DOC2A) c2(XP_011544277) c3(double C2-like domain-containing protein alpha isoform X1) c4(343432) c5(343433, 343434, 343435, 343436, 343437, 343438) c6( )c7(T, Bl, id, ZH, CN, aoc, cF, eL, dL, m, ci, t, nJ, ae, ad, bN); #c1(DOC2B) c2(NP_003576) c3 (double C2-like domain-containing protein beta) c4(139312) c5(139313, 139314, 139315, 139316, 139317, 139318, 139319, 139320, 139321, 139322) c6( )c7(if, il, qN); #c1(DOCK1) c2(NP_001371) c3(dedicator of cytokinesis protein 1 isoform 2) c4(117398) c5(117399, 117400, 117401, 117402, 117403, (17404) c6( )c7(bM, T, ac, mQ, aA, aF, aJu, m, MO, t, lB, eA); #c1(DOCK10) c2(NP_001277192) c3(dedicator of cytokinesis protein 10 DOCK10.2) c4(109210) c5(109211, 109212, 109213, 109214, 109215, 109216) c6( )c7(Y, ey, cj); #c1(DOCK10) c2(NP_055504) c3(dedicator of cytokinesis protein 10 DOCK10.1) c4(181607) c5(181608, 181609, 181610, 181611, 181612, 181613) c6( )c7(Y, ey, cj); #c1(DOCK10) c2(XP_006712685) c3(dedicator of cytokinesis protein 10 isoform X8) c4( )c5(286791) c6( )c7(Y, ey, cj); #c1(DOCK10) c2(XP_006712687) c3(dedicator of cytokinesis protein 10 isoform X10) c4(286792) c5(286793, 286794, 286795, 286796, 286797) c6( )c7(Y, ey, cj); #c1(DOCK11) c2(NP_653259) c3(dedicator of cytokinesis protein 11) c4(239379) c5(239380, 239381, 239382, 239383, 239384, 239385) c6( )c7(aj, J, hX, qC, nv, Qg, ajv, aT, adn, YL, qZ, aW, Pp, fq, bN, W, qe, aiM, cj, aez, pw, ER, be); #c1(DOCK2) c2(NP_004937) c3(dedicator of cytokinesis protein 2) c4(151273, 151274) c5(151275, 151276, 151277, 151278, 151279, 151280, 151281, 151282, 151283, 151284, 151285, 151286, 151287) c6( )c7(ck, bM, ac, pf, aw, U, cd, ey, af); #c1(DOCK2) c2(XP_011532752) c3(dedicator of cytokinesis protein 2 isoform X3) c4(328831) c5(328832, 328833, 328834, 328835, 328836) c6( )c7(ck, bM, ac, pf, aw, U, cd, ey, af); #c1(DOCK3) c2(NP_004938) c3(dedicator of cytokinesis protein 3) c4(151288) c5(151289, 151290, 151291, 151292, 151293, 151294, 151295) c6( )c7(HQ, T, aW, ds, Y, pP, aT, nw, K, aAU, axp, aDF); #c1(DOCK3) c2(XP_006713073) c3(dedicator of cytokinesis protein 3 isoform X8) c4(287016) c5( )c6( )c7(HQ, T, aW, ds, Y, pP, aT, nw, K, aAU, axp, aDF); #c1(DOCK3) c2(XP_011531747) c3(dedicator of cytokinesis protein 3 isoform X12) c4( )c5(327833, 327834, 327835, 327836, 327837) c6( )c7(HQ, T, aW, ds, Y, pP, aT, nw, K, aAU, axp, aDF); #c1(DOCK4) c2(NP_055520) c3(dedicator of cytokinesis protein 4) c4(181690, 181691, 181692) c5(181693, 181694, 181695, 181696, 181697, 181698, 181699, 181700, 181701, 181702, 181703, 181704) c6( )c7(xQ, T, De, EQ, bw, oX, ci, m, yA, t, bN); #c1(DOCK4) c2(XP_006716251) c3(dedicator of cytokinesis protein 4 isoform X1) c4(290198, 290199) c5(290200, 290201, 290202, 290203, 290204, 290205, 290206, 290207, 290208, 290209, 290210, 290211) c6( )c7(xQ, T, oe, EQ, bw, oX, ci, m, yA, t, bN); #c1(DOCK4) c2(XP_006716252) c3(dedicator of cytokinesis protein 4 isoform X3) c4(290212) c5(290213, 290214, 290215, 290216, 290217, 290218) c6( )c7(xQ, T, oe, EQ, bw, oX, ci, m, yA, t, bN); #c1(DOCK4) c2(XP_011515019) c3(dedicator of cytokinesis protein 4 isoform X4) c4( )c5(307794, 307795, 307796, 307797, 307798, 307799) c6( )c7(xQ, T, De, EQ, bw, oX, ci, m, yA, t, bN); #c1(DOCK5) c2(NP_079216) c3(dedicator of cytokinesis protein 5) c4(217473, 217474) c5(217475, 217476, 217477, 217478, 217479, 217480) c6( )c7(v, aw, qM); #c1(DOCK5) c2(XP_011542965) c3 (dedicator of cytokinesis protein 5 isoform X3) c4( )c5(341663, 341664, 341665, 341666, 341667, 341668) c6( )c7(v, aw, qM); #c1(DOCK6) c2(NP_065863) c3(dedicator of cytokinesis protein 6) c4(205951) c5(205952, 205953, 205954, 205955, 205956, 205957) c6( ) c7(afG, bhn, j, bt); #c1(DOCK6) c2(XP_005260058) c3(dedicator of cytokinesis protein 6 isoform X3) c4(276428) c5(276429, 276430, 276431, 276432, 276433, 276434) c6( )c7(afG, bhn, j, bt); #c1(DOCK7) c2(XP_011540628) c3(dedicator of cytokinesis protein 7 isoform X1) c4(339172) c5(339173, 339174, 339175, 339176, 339177, 339178) c6( )c7(bho, d, lm, lE); #c1(DOCK8) c2(NP_982272) c3(dedicator of cytokinesis protein 8 isoform 1) c4(263250, 263251) c5(263252, 263253, 263254, 263255, 263256, 263257, 263258, 263259, 263260, 263261, 263262, 263263, 263264) c6( )c7(oO, bbv, T, nA, cd, bhq, lg, aT, Nr, Cw, aW, oV, CM, aeS, CF, lo, Xm, eE, bz, Nq, cT, Qu, zA, bhp); #c1(DOCK8) c2(XP_011516347) c3(dedicator of cytokinesis protein 8 isoform X1) c4(309574) c5(309575, 309576, 309577, 309578, 309579) c6( )c7(oO, bbv, T, nA, cd, bhq, lg, aT, Nr, Cw, aW, oV, CM, aeS, CF, lo, Xm, eE, bz, Nq, cT, Qu, zA, bhp); #c1 (DOCK8) c2(XP_011516348) c3(dedicator of cytokinesis protein 8 isoform X3) c4( )c5(309580) c6( )c7(oO, bbv, T, nA, cd, bhq, lg, aT, Nr, Cw, aW, oV, CM, aeS, CF, lo, Xm, eE, bz, Nq, cT, Qu, zA, bhp); #c1(DOCK8) c2(XP_011516350) c3(dedicator of cytokinesis protein & isoform X2) c4(309581) c5(309582, 309583, 309584, 309585, 309586) c6( )c7(oO, bbv, T, nA, cd, bhq, lg, aT, Nr, Cw, aW, oV, CM, aeS, CF, lo, Xm, eE, bz, Nq, cT, Qu, zA, bhp); #c1(DOCK9) c2(NP_001123520) c3(dedicator of cytokinesis protein 9 isoform b) c4(47312) c5(47313, 47314, 47315, 47316, 47317, 47318) c6( )c7(d, cj, j, bhr, bk, cd); #c1(DOCK9) c2(NP_001123521) c3(dedicator of cytokinesis protein 9 isoform c) c4( )c5(47319, 47320, 47321, 47322, 47323, 47324) c6( )c7(d, cj, j, bhr, bk, cd); #c1(DOCK9) c2(NP_001123522) c3(dedicator of cytokinesis protein 9 isoform d) c4(47325) c5(47326, 47327, 47328, 47329, 47330, 47331) c6( )c7(d, cj, j, bhr, bk, cd); #c1(DOCK9) c2(XP_005254091) c3(dedicator of cytokinesis protein 9 isoform X16) c4( )c5(272921) c6( )c7(d, cj, j, bhr, bK, cd); #c1(DOCK9) c2(XP_005254092) c3(dedicator of cytokinesis protein 9 isoform X19) c4(272922, 272923) c5(272924, 272925, 272926, 272927, 272928, 272929, 272930, 272931, 272932, 272933, 272934, 272935) c6( )c7(d, cj, j, bhr, bk, cd); #c1(DOCK9) c2(XP_005254093) c3(dedicator of cytokinesis protein 9 isoform X20) c4(272936) c5(272937, 272938, 272939, 272940, 272941) c6( )c7(d, cj, j, bhr, bk, cd); #c1(DOCK9) c2(XP_006719985) c3(dedicator of cytokinesis protein 9 isoform X1) c4(293117) c5(293118, 293119, 293120, 293121, 293122, 293123, 293124) c6( )c7(d, cj, j, bhr, bk, cd); #c1(DOCK9) c2(XP_006719989) c3(dedicator of cytokinesis protein 9 isoform X5) c4(293125) c5(293126, 293127, 293128, 293129, 293130, 293131) c6( )c7(d, cj, j, bhr, bk, cd); #c1(DOCK9) c2(XP_006719992) c3(dedicator of cytokinesis protein 9 isoform X9) c4(293132, 293133) c5(293134, 293135, 293136, 293137, 293138, 293139, 293140, 293141, 293142, 293143, 293144) c6( )c7(d, cj, j, bhr, bK, cd); #c1(DOCK9) c2(XP_006719994) c3(dedicator of cytokinesis protein 9 isoform X11) c4(293145, 293146, 293147) c5(293148, 293149, 293150, 293151, 293152, 293153, 293154, 293155, 293156, 293157, 293158, 293159, 293160, 293161, 293162, 293163, 293164, 293165) c6( )c7(d, cj, j, bhr, bK, cd); #c1(DOCK9) c2(XP_006719995) c3(dedicator of cytokinesis protein 9 isoform X12) c4(293166) c5(293167, 293168, 293169, 293/70, 293171, 293172) c6( )c7(d, cj, j, bhr, bK, cd); #c1(DOCK9) c2(XP_006720000) c3(dedicator of cytokinesis protein 9 isoform X17) c4(293173, 293174, 293175) c5(293176, 293177, 293178, 293179, 293180, 293181, 293182, 293183, 293184, 293185, 293186, 293187, 293188) c6( )c7(d, cj, j, bhr, bk, cd); #c1(DOCK9) c2(XP_006720001) c3 (dedicator of cytokinesis protein 9 isoform X18) c4(293189) c5(293190, 293191, 293192, 293193, 293194, 293195) c6( )c7(d, cj, j, bhr, bk, cd); #c1(DOHH) c2(XP_011526642) c3(deoxyhypusine hydroxylase isoform X1) c4(320949) c5(320950, 320951, 320952, 320953, 320954, 320955, 320956, 320957, 320958, 320959) c6( )c7(ae, d, fM, ad, dV); #c1(DOK1) c2(NP_001184189) c3(docking protein 1 isoform 2) c4( )c5(75557, 75558, 75559, 75560, 75561) c6( )c7(ck, av, T, kP, iU, jh, cj, aA, pk, dz, aF, m, t, B); #c1(DOK1) c2(NP_001372) c3(docking protein 1 isoform 1) c4(1/7405) c5(117406, 117407, 117408, 117409, 117410, 117411) c6( )c7(ck, av, T, kP, il, jh, cj, aA, pk, dz, aF, m, t, B); #c1(DOK1) c2(XP_011530971) c3(docking protein 1 isoform X1) c4(326930) c5( )c6( )c7(ck, av, T, kP, iU, jh, cj, aA, pk, dz, aF, m, t, B); #c1(DOK1) c2(XP_011530972) c3(docking protein 1 isoform X2) c4( )c5(326931) c6( )c7(ck, av, T, kP, iU, jh, cj, aA, pk, dz, aF, m, t, B); #c1(DOK2) c2(NP_003965) c3(docking protein 2) c4(142493, 142494) c5(142495, 142496, 142497, 142498, 142499, 142500, 142501) c6( )c7(ck, av, il, JI, aA, n, aF, m, bX, t, bz, iu, zn); #c1(DOK3) c2(NP_001138347) c3(docking protein 3 isoform 2) c4(56199, 56200) c5(56201, 56202, 56203, 56204, 56205, 56206, 56207, 56208, 56209, 56210, 56211, 56212, 56213, 56214, 56215, 56216, 56217, 56218, 56219, 56220, 56221, 56222, 56223, 56224, 56225, 56226, 56227, 56228, 56229, 56230, 56231, 56232, 56233) c6( )c7(av); #c1(DOK3) c2(NP_001138348) c3(docking protein 3 isoform 3) c4(56234) c5(56235, 56236, 56237, 56238, 56239, 56240) c6( )c7(av); #c1(DOK3) c2(NP_079148) c3(docking protein 3 isoform 1) c4(217244, 217245, 217246, 217247, 217248, 217249) c5(217250, 217251, 217252, 217253, 217254, 217255, 217256, 217257, 217258, 217259, 217260, 217261, 217262, 217263) c6( )c7(av); #c1(DOK3) c2(XP_011532959) c3(docking protein 3 isoform X3) c4( )c5(329120) c6( )c7(av); #c1(DOK4) c2(NP_060580) c3(docking protein 4) c4(196666) c5(196667, 196668, 196669, 196670, 196671, 196672) c6( )c7(av, bz, py, eM); #c1(DOK5) c2(NP_060901) c3(docking protein 5 isoform a) c4(198510) c5(198511, 198512, 198513, 198514, 198515, 198516) c6( )c7(bL, W, v, bK, pb, sj); #c1(DOK6) c2(NP_689934) c3(docking protein 6) c4(244382) c5(244383, 244384, 244385, 244386, 244387, 244388) c6( )c7(bA); #c1(DOK7) c2(NP_001158145) c3(protein Dok-7 isoform 2) c4(63183) c5(63184, 63185, 63186, 63187, 63188) c6( )c7(xC, xm, T, hM, no, Mc, ali, xA, aW, xj, bhs, bht, oj, aT, Ak, aDx); #c1(DOK7) c2(NP_001288000) c3(protein Dok-7 isoform 4) c4(113359) c5(113360, 113361, 113362, 113363, 113364, 113365) c6( )c7(xC, xm, T, hM, no, Mc, ali, xA, aW, xj, bhs, bht, oj, aT, Ak, aDx); #c1(DOK7) c2(NP_775931) c3 (protein Dok-7 isoform 1) c4(250748) c5(250749, 250750, 250751, 250752, 250753, 250754, 250755) c6( )c7(xC, xm, T, hM, no, Mc, ali, xA, aW, xj, bhs, bht, oj, aT, Ak, aDx); #c1(DOK7) c2(XP_011511739) c3(protein Dok-7 isoform X3) c4( )c5(302455) c6( )c7(xC, xm, T, hM, no, Mc, ali, xA, aW, xj, bhs, bht, oj, aT, Ak, aDx); #c1(DOLK) c2(NP_055723) c3(dolichol kinase) c4(183104, 183105) c5(183106, 183107, 183108, 183109, 183110, 183111, 183112, 183113, 183114, 183115, 183116, 183117) c6( )c7(bhu, mx); #c1(DONSON) c2(NP_060083) c3(protein downstream neighbor of Son) c4(194071) c5(194072, 194073, 194074, 194075, 194076, 194077) c6( )c7(ae, ad); #c1(DOPEY2) c2(XP_006724131) c3(protein dopey-2 isoform X1) c4(295797, 295798) c5(295799, 295800, 295801, 295802, 295803, 295804, 295805, 295806, 295807, 295808, 295809, 295810) c6( )c7(f, anj, nA); #c1(DOT1L) c2(NP_115871) c3(histone-lysine N-methyltransferase, H3 lysine-79 specific) c4(224411) c5(224412, 224413, 224414, 224415, 224416, 224417) c6( )c7(ck, T, mx, r, oX, mo, q); #c1(DOT1L) c2(XP_005259717) c3(histone-lysine N-methyltransferase, H3 lysine-79 specific isoform X1) c4(276240) c5(276241, 276242, 276243, 276244, 276245, 276246) c6( )c7(ck, T, mx, r, oX, mo, q); #c1(DPAGT1) c2(NP_001373) c3(UDP-N-acetylglucosamine-dolichyl-phosphate N-acetylglucosaminephosphotransferase) c4(117412) c5(117413, 117414, 117415, 117416, 117417, 117418, 117419, 117420, 117421, 117422) c6( )c7(WI, xA, no, ff, SO, bhw, bu, aA, aQQ, aeu, aq, Km, beZ, bhv, avG, lM, en, B); #c1(DPAGT1) c2(XP_005271479) c3(UDP-N-acetylglucosamine-dolichyl-phosphate N-acetylglucosaminephosphotransferase isoform X1) c4(283430) c5(283431) c6( )c7(WI, xA, no, ff, SO, bhw, bu, aA, aQQ, aeu, aq, Km, beZ, bhv, avG, lM, en, B); #c1(DPAGT1) c2(XP_011540950) c3(UDP-N-acetylglucosamine-dolichyl-phosphate N-acetylglucosaminephosphotransferase isoform X2) c4( )c5(339482, 339483, 339484, 339485, 339486) c6( )c7(WI, xA, no, ff, SO, bhw, bu, aA, aQQ, aeu, aq, Km, beZ, bhv, avG, lM, en, B); #c1(DPCD) c2(NP_056263) c3(protein DPCD) c4(186804, 186805, 186806, 186807, 186808) c5(186809, 186810, 186811, 186812, 186813, 186814, 186815, 186816, 186817, 186818, 186819, 186820, 186821, 186822, 186823, 186824, 186825, 186826, 186827, 186828, 186829, 186830, 186831, 186832, 186833, 186834, 186835, 186836, 186837, 186838, 186839, 186840, 186841) c6( )c7(Pf, MG); #c1(DPCR1) c2(NP_543146) c3 (diffuse panbronchiolitis critical region protein 1 precursor) c4(233402, 233403, 233404, 233405, 233406, 233407) c5(233408, 233409, 233410, 233411, 233412, 233413, 233414, 233415, 233416, 233417, 233418, 233419, 233420, 233421, 233422, 233423, 233424, 233425, 233426, 233427, 233428, 233429, 233430, 233431, 233432, 233433, 233434, 233435, 233436, 233437, 233438, 233439, 233440, 233441, 233442, 233443, 233444, 233445, 233446, 233447, 233448, 233449, 233450, 233451, 233452, 233453, 233454, 233455, 233456, 233457) c6( )c7(aZL, eZ, cr, r, hH, q); #c1(DPEP1) c2(NP_001121613) c3(dipeptidase 1 precursor) c4(45848) c5(45849, 45850, 45851, 45852, 45853, 45854, 45855, 45856, 45857, 45858, 45859, 45860, 45861, 45862) c6( )c7(T, axd, bB, qj, q, ad, AW, aE, aT, aU, ae, s, YY, aW, r, kp, akh, Fq, akw, bz, bN, kg, bE, HP, abh, pC); #c1(DPEP2) c2(NP_07/750) c3(dipeptidase 2 precursor) c4(211932, 211933) c5(211934, 211935, 211936, 211937, 211938, 211939, 211940, 211941, 211942, 211943, 211944, 211945, 211946, 211947, 211948, 211949) c6( )c7(T, id, aA, AD, q, mC, ad, aT, eL, ae, d, s, aW, eZ, la, r, g, pb, ck, Bv, eM, Qu, fL, B); #c1(DPEP2) c2(XP_005256147) c3(dipeptidase 2 isoform X5) c4(274012) c5( )c6( )c7(T, id, aA, AD, q, mC, ad, aT, eL, ae, d, s, aW, eZ, la, r, g, pb, ck, Bv, eM, Qu, FL, B); #c1(DPEP2) c2(XP_011521573) c3(dipeptidase 2 isoform X6) c4( )c5(316305, 316306, 316307, 316308, 316309, 316310) c6( )c7(T, id, aA, AO, q, mC, ad, aT, eL, ae, d, s, aW, eZ, la, r, g, pb, ck, Bv, eM, Qu, FL, B); #c1(DPEP3) c2(NP_07/752) c3(dipeptidase 3 isoform a precursor) c4(211950) c5(211951, 211952, 211953, 211954, 211955, 211956) c6( ) c7(T, eM, aD, qj, bz, pC); #c1(DPF1) c2(NP_001128627) c3(zinc finger protein neuro-d4 isoform a) c4(50375) c5(50376, 50377, 50378, 50379, 50380, 50381, 50382, 50383, 50384, 50385) c6( )c7(ad); #c1(DPF1) c2(NP_001128628) c3(zinc finger protein neuro-d4 isoform c) c4( )c5(50386, 50387, 50388, 50389, 50390) c6( )c7(ad); #c1(DPF1) c2(NP_004638) c3(zinc finger protein neuro-d4 isoform b) c4(148424) c5(148425, 148426, 148427, 148428, 148429, 148430, 148431) c6( )c7(ad); #c1(DPF1) c2(XP_005259347) c3(zinc finger protein neuro-d4 isoform X5) c4(276068, 276069) c5(276070, 276071, 276072, 276073, 276074, 276075, 276076, 276077, 276078, 276079, 276080, 276081, 276082) c6( )c7(ad); #c1(DPF1) c2(XP_006723470) c3(zinc finger protein neuro-d4 isoform X2) c4(295222) c5( )c6( )c7(ad); #c1(DPF3) c2(NP_001267471) c3(zinc finger protein DPF3 isoform 2) c4(99580, 99581) c5(99582, 99583, 99584, 99585, 99586, 99587, 99588, 99589, 99590, 99591, 99592, 99593) c6( )c7(Na, aj, Nc, bX, aT); #c1(DPF3) c2(NP_036206) c3(zinc finger protein DPF3 isoform 1) c4(172378) c5(172379, 172380, 172381, 172382, 172383, 172384, 172385, 172386, 172387, 172388) c6( )c7(Na, aj, Nc, bX, aT); #c1(DPH1) c2(NP_001374) c3(diphthamide biosynthesis protein 1) c4(117423) c5(117424, 117425, 117426, 117427, (17428, 117429) c6( )c7(x, t, m, k); #c1(DPH3) c2(NP_001040899) c3(DPH3 homolog isoform 2) c4(34217) c5(34218, 34219, 34220, 34221, 34222, 34223) c6( )c7(Y); #c1(DPH3) c2(NP_996662) c3(DPH3 homolog isoform 1) c4(264340) c5(264341, 264342, 264343, 264344, 264345, 264346) c6( )c7(Y); #c1(DPH6) c2(NP_001135444) c3(diphthine-ammonia ligase isoform 2) c4(52936) c5(52937, 52938, 52939, 52940, 52941, 52942) c6( )c7(y, aC, c); #c1(DPH6) c2(NP_542381) c3(diphthine-ammonia ligase isoform 1) c4(232309) c5(232310, 232311, 232312, 232313, 232314, 232315) c6( )c7(y, aC, c); #c1(DPH7) c2(NP_620133) c3(diphthamide biosynthesis protein 7) c4(237324) c5(237325, 237326, 237327, 237328, 237329, 237330) c6( )c7(jh); #c1(DPMI) c2(NP_003850) c3(dolichol-phosphate mannosyltransferase subunit 1) c4(141411) c5(141412, 141413, 141414, 141415, 141416, 141417) c6( )c7(aE, Km, dv, SM); #c1(DPM2) c2(NP_003854) c3(dolichol phosphate-mannose biosynthesis regulatory protein) c4(141524, 141525) c5(141526, 141527, 141528, 141529, 141530, 141531, 141532, 141533, 141534, 141535, 141536, 141537) c6( )c7(bhx, ha, xs); #c1(DPM3) c2(NP_061846) c3(dolichol-phosphate mannosyltransferase subunit 3 isoform 1) c4(200754, 200755) c5(200756, 200757, 200758, 200759, 200760, 200761, 200762, 200763) c6( )c7(bhy, dv, ad); #c1(DPM3) c2(NP_714963) c3(dolichol-phosphate mannosyltransferase subunit 3 isoform 2) c4( ) c5(246662, 246663, 246664, 246665, 246666, 246667, 246668, 246669) c6( )c7(bhy, dv, ad); #c1(DPP10) c2(NP_001171505) c3(inactive dipeptidyl peptidase 10 isoform c) c4(70334, 70335, 70336, 70337) c5(70338, 70339, 70340, 70341, 70342, 70343, 70344) c6( )c7(bM, cT, Dw, VX, ac, nO, sp, oe, ml, aq, Ds, v, bK, rz); #c1(DPP10) c2(NP_001171507) c3(inactive dipeptidyl peptidase 10 isoform a) c4( )c5(70352, 70353, 70354, 70355, 70356) c6( )c7(bM, cT, Dw, VX, ac, nO, sp, oe, ml, aq, Ds, v, bK, rz); #c1(DPP10) c2(NP_065919) c3(inactive dipeptidyl peptidase 10 isoform long) c4(206251) c5(206252, 206253, 206254, 206255, 206256, 206257) c6( )c7(bM, cT, Dw, VX, ac, nO, sp, oe, ml, aq, Ds, v, bK, rz); #c1(DPP10) c2(XP_011509828) c3(inactive dipeptidyl peptidase 10 isoform X1) c4(300415) c5(300416, 300417, 300418, 300419, 300420, 300421, 300422) c6( )c7(bM, cT, Dw, VX, ac, nO, sp, oe, ml, aq, Ds, v, bK, rz); #c1(DPP10) c2(XP_011509829) c3(inactive dipeptidyl peptidase 10 isoform X2) c4( )c5(300423, 300424, 300425, 300426, 300427, 300428, 300429, 300430, 300431, 300432, 300433) c6( )c7(bM, cT, Dw, VX, ac, nO, sp, oe, ml, aq, Ds, v, bK, rz); #c1(DPP3) c2(NP_001243599) c3(dipeptidyl peptidase 3 isoform 2) c4( )c5(91134, 91135, 91136, 91137, 91138, 91139) c6( )c7(T); #c1(DPP3) c2(NP_569710) c3(dipeptidyl peptidase 3 isoform 1) c4(233741, 233742, 233743, 233744, 233745, 233746, 233747) c5(233748, 233749, 233750, 233751, 233752, 233753, 233754, 233755, 233756, 233757, 233758, 233759, 233760, 233761, 233762, 233763, 233764, 233765, 233766, 233767, 233768, 233769, 233770, 233771, 233772, 233773, 233774, 233775, 233776, 233777, 233778, 233779, 233780, 233781, 233782, 233783, 233784, 233785, 233786, 233787, 233788, 233789, 233790, 233791) c6( )c7(T); #c1(DPP4) c2(NP_001926) c3(dipeptidyl peptidase 4) c4(122253) c5(122254, 122255, 122256, 122257, 122258, 122259) c6( )c7(T, fR, ea, e, al, aza, bB, fd, cl, ci, ip, GK, iX, qM, wt, aW, g, eE, qJ, eM, iA, cj, m, dD, aw, Wb, aj, fr, ct, ai, cc, vM, h, jy, eV, dx, kK, fe, Wt, hq, hk, uz, eo, AN, bL, fM, dA, AS, eX, cz, v, j, C, V, q, J, eA, aT, aFm, ae, il, De, nO, oV, Y, xR, r, lo, hf, t, ck, iM, mf, dy, hS, iW, jh, bS, eq, dG, ad, wD, dP, aHF, hd, F, ib, dk, lQ, fb, sj, je, wc, hp, Z, bz, es, gk, eU, bE, CO, bhz, ei, X, Cp): c1(DPP6) c2(NP_001034439) c3(dipeptidyl aminopeptidase-like protein 6 isoform 3) c4(3/7/7) c5(3/718, 31719, 31720, 31721, 31722, 31723) c6( )c7(aC, rR, bhA, VX, bhB, c, aV, ms, arD, vc, Vd, rz, bt); #c1(DPP6) c2(NP_001277181) c3(dipeptidyl aminopeptidase-like protein 6 isoform 4) c4(109201) c5(109202, 109203, 109204, 109205, 109206, 109207) c6( )c7(aC, rR, bhA, VX, bhB, c, aV, ms, arD, vc, Vd, rz, bt); #c1(DPP6) c2(NP_001277182) c3(dipeptidyl aminopeptidase-like protein 6 isoform 5) c4( )c5(109208) c6( )c7(aC, rR, bhA, VX, bhB, c, aV, ms, arD, vc, Vd, rz, bt); #c1(DPP6) c2(NP_001927) c3(dipeptidyl aminopeptidase-like protein 6 isoform 2) c4(122260) c5(122261, 122262, 122263, 122264, 122265, 122266) c6( )c7(aC, rR, bhA, VX, bhB, c, aV, ms, arD, vc, Vd, rz, bt); #c1(DPP6) c2(NP_570629) c3(dipeptidyl aminopeptidase-like protein 6 isoform 1) c4(234128) c5(234129, 234130, 234131, 234132, 234133, 234134) c6( )c7(aC, rR, bhA, VX, bhB, c, aV, ms, arD, vc, Vd, rz, bt); #c1(DPP7) c2(NP_037511) c3(dipeptidyl peptidase 2 preproprotein) c4(175947) c5(175948, 175949, 175950, 175951, 175952, 175953, 175954, 175955, 175956) c6(175957, 175958) c7(cj); #c1(DPP7) c2(XP_006717146) c3(dipeptidyl peptidase 2 isoform X3) c4( ) c5(290781) c6( )c7(cj); #c1(DPP7) c2(XP_011516903) c3(dipeptidyl peptidase 2 isoform X5) c4(310353) c5(310354, 310355, 310356, 310357, 310358, 310359, 310360) c6(310361, 310362) c7(cj); #c1(DPP8) c2(NP_060213) c3(dipeptidyl peptidase 8 isoform 2) c4( ) c5(194776, 194777, 194778, 194779, 194780) c6( )c7(T, dD, aW, ea, aT, eq, m, ip); #c1(DPP8) c2(NP_932064) c3(dipeptidyl peptidase 8 isoform 3) c4(258747, 258748, 258749) c5(258750, 258751, 258752, 258753, 258754, 258755, 258756, 258757, 258758, 258759, 258760, 258761, 258762, 258763, 258764, 258765, 258766, 258767, 258768, 258769, 258770, 258771) c6( )c7(T, dD, aW, ea, aT, eq, m, ip); #c1(DPP8) c2(NP_932065) c3(dipeptidyl peptidase 8 isoform 4) c4(258772) c5(258773, 258774, 258775, 258776, 258777, 258778) c6( )c7(T, dD, aW, ea, aT, eq, m, ip); #c1(DPP8) c2(XP_005254562) c3(dipeptidyl peptidase 8 isoform X8) c4( )c5(273251, 273252, 273253, 273254, 273255, 273256) c6( )c7(T, dD, aW, ea, aT, eq, m, ip); #c1(DPP8) c2(XP_011520033) c3(dipeptidyl peptidase 8 isoform X3) c4(314961) c5( )c6( )c7(T, dD, aW, ea, aT, eq, m, ip); #c1(DPP8) c2(XP_011520036) c3(dipeptidyl peptidase 8 isoform X6) c4(314962) c5(314963) c6( )c7(T, dD, aW, ea, aT, eq, m, ip); #c1(DPP9) c2(XP_005259730) c3(dipeptidyl peptidase 9 isoform X1) c4(276264, 276265, 276266) c5(276267, 276268, 276269, 276270, 276271, 276272, 276273, 276274, 276275, 276276, 276277, 276278) c6( )c7(LB, dD, ea, cj, eq, bQ, ip); #c1(DPP9) c2(XP_011526711) c3(dipeptidyl peptidase 9 isoform X4) c4( )c5(321021) c6( ) c7(LB, dD, ea, cj, eq, bQ, ip); #c1(DPP9) c2(XP_011526713) c3(dipeptidyl peptidase 9 isoform X5) c4( )c5(321022, 321023, 321024, 321025, 321026, 321027) c6( )c7(LB, dD, ea, cj, eq, bQ, ip); #c1(DPPA2) c2(NP_620170) c3(developmental pluripotency-associated protein 2) c4( )c5(237459, 237460, 237461, 237462, 237463) c6( )c7(av, TK, q, r); #c1(DPPA2) c2(XP_006713572) c3(developmental pluripotency-associated protein 2 isoform X2) c4(287238) c5(287239) c6( )c7(av, TK, q, r); #c1(DPPA3) c2(NP_954980) c3(developmental pluripotency-associated protein 3) c4(261498) c5(261499, 261500, 261501, 261502, 261503, 261504, 261505) c6( ) c7(Lb); #c1(DPPA4) c2(NP_060659) c3(developmental pluripotency-associated protein 4) c4(197022) c5(197023, 197024, 197025, 197026, 197027, 197028) c6( )c7(wu, wm); #c1(DPT) c2(NP_001928) c3(dermatopontin precursor) c4(122267) c5(122268, 122269, 122270, 122271, 122272, 122273) c6( )c7(bA, gj, T, A, op, zt, e, vA, aep, cz, Mz, aj, Hr, ci, g, ae, eW, J, ad, afu); #c1(DPY19L2) c2(NP_776173) c3(probable C-mannosyltransferase DPY19L2) c4(250932) c5(250933, 250934, 250935, 250936, 250937, 250938) c6( )c7(bhC, gU); #c1(DPY19L2) c2(XP_006719413) c3(probable C-mannosyltransferase DPY19L2 isoform X2) c4(292466) c5( )c6( )c7(bhC, gU); #c1(DPY19L2) c2(XP_006719419) c3(probable C-mannosyltransferase DPY19L2 isoform X8) c4(292467) c5( )c6( )c7(bhC, gU); #c1(DPY19L2) c2(XP_011536519) c3(probable C-mannosyltransferase DPY19L2 isoform X7) c4( )c5(333880, 333881, 333882, 333883, 333884, 333885) c6( )c7(bhC, gU); #c1(DPY19L2) c2(XP_011536520) c3(probable C-mannosyltransferase DPY19L2 isoform X9) c4( ) c5(333886, 333887, 333888, 333889, 333890, 333891) c6( )c7(bhC, gU); #c1(DPY19L3) c2(XP_011524828) c3(probable C-mannosyltransferase DPY19L3 isoform X1) c4(319253) c5(319254, 319255, 319256, 319257, 319258, 319259, 319260, 319261, 319262, 319263) c6( )c7(bK); #c1(DPY19L3) c2(XP_011524829) c3(probable C-mannosyltransferase DPY19L3 isoform X2) c4(319264, 319265) c5(319266, 319267, 319268, 319269, 319270, 319271, 319272, 319273, 319274, 319275, 319276, 319277) c6( )c7(bK); #c1(DPY30) c2(NP_115963) c3(protein dpy-30 homolog) c4(224963) c5(224964, 224965, 224966, 224967, 224968, 224969, 224970, 224971, 224972, 224973) c6( )c7(bhD, ck); #c1(DPYS) c2(NP_001376) c3(dihydropyrimidinase) c4(117430) c5(117431, 117432, 117433, 117434, 117435, 117436) c6( )c7(IJ, T, W, nb, bhF, HT, oj, hl, aA, ayr, aF, cm, cd, bhE); #c1(DPYSL2) c2(NP_001184222) c3 (dihydropyrimidinase-related protein 2 isoform 1) c4(75562) c5(75563) c6( )c7(T, iH, De, ci, il, qi, V, aPQ, WY, aT, bK, he, af, eu, aW, gl, aaE, f, cm, bz, fq, eM, bE, U, gT); #c1(DPYSL2) c2(NP_001231533) c3(dihydropyrimidinase-related protein 2 isoform 3) c4(87284) c5(87285, 87286, 87287, 87288, 87289, 87290, 87291, 87292, 87293, 87294, 87295, 87296, 87297, 87298) c6( )c7(T, iH, oe, ci, il, qi, V, aPQ, WY, aT, bK, he, af, eu, aW, gl, aaE, f, cm, bz, fq, eM, bE, U, gT); #c1(DPYSL2) c2(NP_001377) c3(dihydropyrimidinase-related protein 2 isoform 2) c4(117437) c5(117438, 117439, 117440, 117441, 117442, 117443, 117444, 117445, 117446, 117447, 117448) c6( )c7(T, iH, oe, ci, il, qi, V, aPQ, WY, aT, bK, he, af, eu, aW, gl, aaE, f, cm, bz, fq, eM, bE, U, gT); #c1(DPYSL3) c2(NP_001184223) c3(dihydropyrimidinase-related protein 3 isoform 1) c4(75564) c5(75565, 75566, 75567, 75568, 75569, 75570) c6( )c7(ae, hl, aE, wl, ha, cm, Xj, K, f, bz, ad); #c1(DPYSL3) c2(NP_001378) c3(dihydropyrimidinase-related protein 3 isoform 2) c4(117449, 117450) c5(117451, 117452, 117453, 117454, 117455, 117456, 117457, 117458, 117459, 117460, 117461, 117462) c6( )c7(ae, hl, aE, wO, ha, cm, Xj, K, f, bz, ad); #c1(DPYSL3) c2(XP_011535876) c3 (dihydropyrimidinase-related protein 3 isoform X1) c4(333145) c5(333146, 333147, 333148, 333149, 333150, 333151) c6( )c7(ae, hl, aE, wl, ha, cm, Xj, K, f, bz, ad); #c1(DPYSL4) c2(NP_006417) c3(dihydropyrimidinase-related protein 4) c4(164858, 164859) c5(164860, 164861, 164862, 164863, 164864, 164865, 164866) c6( )c7(f, bhG); #c1(DPYSL4) c2(XP_011537427) c3(dihydropyrimidinase-related protein 4 isoform X1) c4( ) c5(335072, 335073, 335074, 335075, 335076, 335077, 335078, 335079, 335080) c6( )c7(f, bhG); #c1(DPYSL5) c2(NP_001240652) c3(dihydropyrimidinase-related protein 5) c4(88647, 88648, 88649, 88650) c5(88651, 88652, 88653, 88654, 88655, 88656, 88657, 88658, 88659, 88660, 88661, 88662, 88663, 88664, 88665, 88666, 88667, 88668, 88669, 88670, 88671, 88672, 88673, 88674, 88675, 88676, 88677, 88678) c6( )c7(av, ft, apo, nG, vd, pd, abW, mH); #c1(DRAM1) c2(NP_060840) c3(DNA damage-regulated autophagy modulator protein 1) c4(198085) c5(198086, 198087, 198088, 198089, 198090, 198091) c6( )c7(T, d, iR, eH, cz, aq, K, jj, eF, B); #c1(DRAP1) c2(NP_006433) c3(drl-associated corepressor) c4(165203) c5(165204, 165205, 165206, 165207, 165208, 165209, 165210) c6( )c7(Y); #c1(DRC1) c2(NP_659475) c3(dynein regulatory complex protein 1) c4(240098) c5(240099, 240100, 240101, 240102, 240103, 240104) c6( )c7(MG, bhH); #c1(DRC1) c2(XP_005264694) c3(dynein regulatory complex protein 1 isoform X1) c4(279207) c5( ) c6( )c7(MG, bhH); #c1(DRC1) c2(XP_005264695) c3(dynein regulatory complex protein 1 isoform X2) c4( )c5(279208, 279209, 279210, 279211, 279212, 279213) c6( )c7(MG, bhH); #c1(DRD1) c2(NP_000785) c3(D(IA) dopamine receptor) c4(15732) c5(15733, 15734, 15735, 15736, 15737, 15738, 15739, 15740, 15741, 15742) c6( )c7(bL, alk, eR, wu, oe, uv, cz, mS, Jd, uu, ct, dG, v, ww, VV, ip, fU, HF, cF, hw, mW, XY, aU, h, bdp, dL, bK, K, wm, bq, oi, mY, mV, an, xa, af, hZ, mU, yL, fO, gC, gl, cu, abn, aFx, HL, Eg, bu, cm, xw, jb, mZ, qa, FT, mX, cv, ail, Kc, op, bN, abV, cA, rR, vA, Gs, bhl, at, sv, ei, jd, mT); #c1(DRD2) c2(NP_000786) c3(D(2) dopamine receptor isoform long) c4(15743, 15744, 15745) c5(15746, 15747, 15748, 15749, 15750, 15751, 15752, 15753, 15754, 15755, 15756, 15757, 15758, 15759, 15760, 15761, 15762, 15763, 15764, 15765, 15766, 15767, 15768, 15769, 15770, 15771) c6( )c7(Gk, T, uk, aUk, e, bO, gf, ur, HH, VV, aR, aji, bhN, iX, Gb, aU, K, qM, oi, xa, gr, KC, yo, aW, cm, mZ, aiO, bdc, eM, vA, ZT, Gs, sv, uJ, m, FW, lT, bhT, ht, n, To, aj, ww, ct, nA, fK, bhM, ke, no, hw, bhR, aQQ, h, aNU, bdp, gl, aPe, dL, Gw, mV, mY, he, ml, abf, PT, Fn, Jj, ge, fe, vo, op, mX, cv, hq, aap, bhK, abV, ok, cA, yP, Ei, bhU, VU, hR, at, aqm, lB, jd, bhP, mT, qc, bL, iH, rG, eR, hU, bhS, He, qQ, hx, cz, mS, ti, hy, v, avO, bhJ, V, q, J, cF, DK, QU, aql, aT, cy, qv, aaw, bK, bq, an, rE, aqC, af, eu, s, Y, yL, fO, bcD, r, cu, HL, t, xw, FT, tl, bN, pH, rR, bhQ, Fl, gO, GI, VR, iW, ha, VT, Hn, Gd, bhL, bS, uu, dG, OH, KL, abZ, dP, HF, aDE, ot, mW, UG, aqk, Sb, na, Jc, hZ, d, aal, Wi, aCt, Gv, gC, gl, iV, Z, FX, jb, bz, iq, gk, bhO, HP, Rm, bC, ei, Gp, Gu, ne); #c1(DRD2) c2(NP_057658) c3(D(2) dopamine receptor isoform short) c4(192134, 192135, 192136) c5(192137, 192138, 192139, 192140, 192141, 192142, 192143, 192144, 192145, 192146, 192147, 192148, 192149, 192150, 192151, 192152, 192153) c6( )c7(Gk, T, uk, aUk, e, bO, gf, ur, HH, VV, aR, aji, bhN, iX, Gb, aU, K, qM, oi, xa, gr, KC, yo, aW, cm, mZ, aiO, bdc, eM, vA, ZT, Gs, sv, uJ, m, FW, lT, bhT, ht, n, To, aj, ww, ct, nA, fK, bhM, ke, no, hw, bhR, aQQ, h, aNU, bdp, gl, aPe, dL, Gw, mV, mY, he, ml, abf, PT, Fn, Jj, ge, fe, vo, op, mX, cv, hq, aap, bhK, abV, ok, cA, yP, Ei, bhU, VU, hR, at, aqm, lB, jd, bhP, mT, qc, bL, iH, rG, eR, hU, bhS, He, qQ, hx, cz, mS, ti, hy, v, avO, bhJ, V, q, J, cF, DK, QU, aql, aT, cy, qv, aaw, bK, bq, an, rE, aqC, af, eu, s, Y, yL, fO, bcD, r, cu, HL, t, xw, FT, tl, bN, pH, rR, bhQ, Fl, gO, GI, VR, iW, ha, VT, Hn, Gd, bhL, bS, uu, dG, OH, KL, abZ, dP, HF, aQE, ot, mW, UG, aqk, Sb, na, Jc, hZ, d, aal, Wi, aCt, Gv, gC, gl, iV, Z, FX, jb, bz, iq, gk, bhO, HP, Rm, bC, ei, Gp, Gu, ne); #c1(DRD3) c2(NP_001269492) c3(D(3) dopamine receptor isoform a) c4(101741, 101742) c5(101743, 101744, 101745, 101746, 101747, 101748, 101749, 101750, 101751, 101752, 101753, 101754, 101755, 101756, 101757, 101758, 101759, 101760, 101761, 101762) c6( )c7(qX, iH, aUk, aZ, iW, He, eX, bO, cz, mS, ti, bS, uu, ct, ww, HH, V, VV, bhW, HF, PW, Gt, bhV, mW, iX, XY, UG, aT, h, eV, Sb, bK, an, mY, mV, he, aTO, xa, na, af, rz, mu, bN, PT, kl, hZ, Jj, aal, aW, yL, bcD, r, gl, cu, HL, PG, Eg, Z, vo, xw, jb, mZ, FT, mX, cv, aiO, aUc, hq, bhX, ok, akx, cA, rR, dJ, yP, Gs, sv, dy, jd, aqD, mT, qc); #c1(DRD3) c2(NP_387512) c3(D(3) dopamine receptor isoform e) c4(229024) c5(229025, 229026, 229027, 229028, 229029, 229030) c6( )c7(qX, iH, alk, aZ, iW, He, eX, bO, cz, mS, ti, bS, uu, ct, ww, HH, V, VV, bhW, HF, PW, Gt, bhV, mW, iX, XY, UG, aT, h, eV, Sb, bK, an, mY, mV, he, aTO, xa, na, af, rz, ml, bN, PT, kl, hZ, Jj, aal, aW, yL, bcD, r, gl, cu, HL, PG, Eg, Z, vo, xw, jb, mZ, FT, mX, cv, aiO, aUc, hq, bhX, ok, akx, cA, rR, dJ, yP, Gs, sv, dy, jd, aqD, mT, qc); #c1(DRD4) c2(NP_000788) c3(D(4) dopamine receptor) c4(15772) c5(15773, 15774, 15775, 15776, 15777, 15778, 15779, 15780, 15781) c6( )c7(uk, aUk, bO, eb, gf, bhZ, Jd, bhN, bid, iX, Gb, ta, abj, K, xa, uC, mZ, bdc, eM, ZT, Gs, adl, sv, abl, alv, uJ, yA, FW, ww, ct, ml, bhM, no, axc, dL, gl, mY, mV, he, mU, PT, agP, bic, vo, mX, bJ, hq, ok, abV, cA, bP, bhU, at, lB, jd, mT, qc, jv, iH, oe, mS, ti, v, Si, aqt, aql, bk, bia, an, rE, rz, af, jf, Uf, yL, zS, r, HL, f, FT, bN, pH, bdF, bhY, bS, uu, abZ, HF, mW, aqk, na, Jc, agK, hZ, VX, tb, Gv, gC, gl, Z, jb, bz, GB, bib, aUa, Gp); #c1(DRD5) c2(NP_000789) c3(D(IB) dopamine receptor) c4(15782) c5(15783, 15784, 15785, 15786, 15787, 15788) c6( )c7(uk, iH, aUk, iW, mS, bS, uu, ct, ww, aOP, mW, XY, aqk, aU, bK, mT, an, mY, mV, he, af, na, mU, ayn, mZ, FT, mX, dJ, HK, aUZ, aUR, Gp, WI); #c1(DRG1) c2(NP_004138) c3(developmentally-regulated GTP-binding protein 1) c4(143843) c5(143844, 143845, 143846, 143847, 143848, 143849) c6( )c7(ae, T, s, bE, aW, PX, aT, bB, it, dL, g, bz, ad); #c1(DRGX) c2(NP_001263380) c3(dorsal root ganglia homeobox protein) c4(97199, 97200) c5(97201, 97202, 97203, 97204, 97205, 97206, 97207, 97208, 97209, 97210, 97211, 97212) c6( )c7(cd); #c1(DROSHA) c2(NP_001093882) c3(ribonuclease 3 isoform 2) c4(40489, 40490, 40491) c5(40492, 40493, 40494, 40495, 40496, 40497, 40498, 40499, 40500, 40501, 40502, 40503) c6( )c7(T, gU, ht, bs, n, hy, fr, ci, br, iu, vJ, il, dL, hP, K, eL, BN, s, bfz, g, cl, t, bw, bz, ck, es, kP, eM, hL, cj, m, aan, ne); #c1 (DROSHA) c2(NP_037367) c3 (ribonuclease 3 isoform 1) c4(175060) c5(175061, 175062, 175063, 175064, 175065, 175066, 175067, 175068, 175069, 175070) c6( )c7(T, gU, ht, bs, n, hy, fr, ci, br, iu, vJ, il, dl, hP, K, eL, BN, s, bfz, g, cl, t, bw, bz, ck, es, kP, BM, hL, cj, m, aan, ne); #c1(DROSHA) c2(XP_005248351) c3(ribonuclease 3 isoform X4) c4(268324) c5(268325, 268326, 268327, 268328, 268329, 268330) c6( )c7(T, gU, ht, bs, n, hy, fr, ci, br, iu, vJ, il, dL, hP, K, eL, BN, s, bfz, g, cl, t, bw, bz, ck, es, kP, eM, hL, cj, m, aan, ne); #c1(DROSHA) c2(XP_011512335) c3(ribonuclease 3 isoform X5) c4( )c5(302984, 302985, 302986, 302987, 302988, 302989) c6( )c7(T, gU, ht, bs, n, hy, fr, ci, br, iu, vJ, il, dL, hP, K, eL, BN, s, bfz, g, cl, t, bw, bz, ck, es, kP, eM, hl, cj, m, aan, ne); #c1(DRP2) c2(NP_001164655) c3(dystrophin-related protein 2 isoform 2) c4(67148) c5( )c6( )c7(gl, PT, bk); #c1(DRP2) c2(NP_001930) c3(dystrophin-related protein 2 isoform 1) c4(122274) c5(122275, 122276, 122277, 122278, 122279, 122280, 122281, 122282, 122283, 122284, 122285, 122286, 122287, 122288, 122289, 122290, 122291, 122292, 122293, 122294) c6( )c7(gl, PT, bK); #c1(DSC1) c2(NP_004939) c3(desmocollin-1 isoform Dsc1b preproprotein) c4(151296) c5(151297, 151298, 151299, 151300, 151301, 151302) c6( )c7(bbA, Y, ji, g, bie, q, bif); #c1(DSC1) c2(NP_077739) c3(desmocollin-1 isoform Dsc1a preproprotein) c4(215327) c5(215328, 215329, 215330, 215331, 215332, 215333) c6( )c7(bbA, Y, ji, g, bie, q, bif); #c1(DSC2) c2(NP_004940) c3(desmocollin-2 isoform Dsc2b preproprotein) c4(151303) c5(151304, 151305, 151306, 151307, 151308, 151309) c6( )c7(T, bih, ht, by, aA, n, bB, ci, AO, XM, q, ad, cc, aT, ey, K, big, lL, hu, ji, ae, qt, aW, ff, r, akt, aF, en, g, cm, bz, alu, wK, bE, hk, Qu, mx, m, bii, it, X); #c1(DSC2) c2(NP_077740) c3(desmocollin-2 isoform Dsc2a preproprotein) c4(215334, 215335, 215336) c5(215337, 215338, 215339, 215340, 215341, 215342, 215343, 215344, 215345, 215346, 215347, 215348) c6( )c7(T, bih, ht, by, aA, n, bB, ci, AO, XM, q, ad, cc, aT, ey, K, big, lQ, hu, ji, ae, qt, aW, ff, r, akt, aF, en, g, cm, bz, alu, wK, bE, hk, Qu, mx, m, bii, it, X); #c1(DSC2) c2(XP_005258263) c3(desmocollin-2 isoform X1) c4( )c5(275595, 275596, 275597, 275598, 275599, 275600) c6( )c7(T, bih, ht, by, aA, n, bB, ci, AO, XM, q, ad, cc, aT, ey, K, big, lQ, hu, ji, ae, qt, aW, ff, r, akt, aF, en, g, cm, bz, alu, wK, bE, hk, Qu, mx, m, bii, it, X); #c1(DSC3) c2(NP_001932) c3(desmocollin-3 isoform Dsc3a preproprotein) c4(122295) c5(122296, 122297, 122298, 122299, 122300, (22301) c6( )c7(T, bih, by, aA, n, bS, ct, ci, AO, XM, q, ad, sO, aT, ji, K, lQ, ae, qt, aW, ff, r, akt, aF, en, g, Z, t, cm, bz, alu, bbA, Qu, hk, wK, cj, vL, m, bii, it); #c1(DSC3) c2(NP_077741) c3(desmocollin-3 isoform Dsc3b preproprotein) c4(215349) c5(215350, 215351, 215352, 215353, 215354, 215355) c6( )c7(T, bih, by, aA, n, bS, ct, ci, AO, XM, q, ad, sO, aT, ji, K, lQ, ae, qt, aW, ff, r, akt, aF, en, g, Z, t, cm, bz, alu, bbA, Qu, hk, wK, cj, vL, m, bii, it); #c1(DSCAM) c2(NP_001380) c3(Down syndrome cell adhesion molecule isoform CHD2-42 precursor) c4(117463) c5(117464, 117465, 117466, 117467, 117468, 117469) c6( )c7(eM, ha, bD, vH, bK, lm, nA, ahl, j, f, lB, ahE, y); #c1(DSCAML1) c2(NP_065744) c3(Down syndrome cell adhesion molecule-like protein 1) c4(205154) c5(205155, 205156, 205157, 205158, 205159, 205160) c6( )c7(ac); #c1(DSCAML1) c2(XP_011541220) c3(Down syndrome cell adhesion molecule-like protein 1 isoform X2) c4(339860) c5(339861, 339862, 339863, 339864, 339865, 339866) c6( )c7(ac); #c1(DSCC1) c2(NP_076999) c3(sister chromatid cohesion protein DCC1) c4(214673) c5(214674, 214675, 214676, 214677, 214678, 214679) c6( )c7(T, q, r); #c1(DSCR4) c2(NP_005858) c3(Down syndrome critical region protein 4) c4(159983) c5(159984, 159985, 159986, 159987, 159988, 159989) c6( ) c7(f); #c1(DSE) c2(NP_037484) c3(dermatan-sulfate epimerase precursor) c4(175764, 175765, 175766) c5(175767, 175768, 175769, 175770, 175771, 175772, 175773, 175774, 175775, 175776, 175777, 175778, 175779, 175780, 175781, 175782, 175783, 175784, 175785, 175786, 175787, 175788, 175789, 175790, 175791, 175792, 175793) c6( )c7(bij, Eh, ac); #c1(DSEL) c2(NP_115536) c3(dermatan-sulfate epimerase-like protein precursor) c4(223115, 223116) c5(223117, 223118, 223119, 223120, 223121, 223122, 223123, 223124, 223125, 223126, 223127, 223128) c6( )c7(xS, aw); #c1(DSG1) c2(NP_001933) c3(desmoglein-1 preproprotein) c4(122302) c5(122303, 122304, 122305, 122306, 122307, 122308) c6( )c7(T, by, Pz, Tz, aRG, alg, lG, lQ, Zm, bil, axu, Y, ff, aFE, m, dN, dq, akY, aEW, LU, en, f, aRJ, dr, ay, cT, bik, XO, bim, awa); #c1(DSG2) c2(NP_001934) c3(desmoglein-2 preproprotein) c4(122309) c5(122310, 122311, 122312, 122313, 122314, 122315) c6( ) c7(bio, ay, cc, fM, wK, bE, bin, Y, mx, aA, bB, fe, dg, af, ci, iV, bii, pb, wa); #c1(DSG3) c2(NP_001935) c3(desmoglein-3 preproprotein) c4(122316) c5(122317, 122318, 122319, 122320, 122321, 122322) c6( )c7(T, hy, ci, aGX, av, biq, bip, aet, pX, alg, ab, lQ, an, axu, ff, aFE, en, g, pl, pm, ik, bz, ay, eM, Qu, lV, XD); #c1(DSG4) c2(NP_001127925) c3(desmoglein-4 isoform 1 preproprotein) c4(49512) c5(49513, 49514, 49515, 49516, 49517, 49518) c6( )c7(alC, alu, bis, bir, axu, afB, xV, aEW, bim, pL); #c1(DSG4) c2(NP_817123) c3(desmoglein-4 isoform 2 preproprotein) c4(253114) c5(253115, 253116, 253117, 253118, 253119, 253120) c6( )c7(alC, alu, bis, bir, axu, afB, xV, aEW, bim, pl); #c1(DSP) c2(NP_001008844) c3(desmoplakin isoform Il) c4(23437) c5(23438, 23439, 23440, 23441, 23442, 23443, 23444, 23445, 23446, 23447, 23448, 23449) c6( )c7(aOR, T, Ke, by, n, aml, ci, aFJ, biy, XM, cd, Hr, ip, biA, av, cc, biu, biw, bit, ey, lQ, Tj, af, biz, Y, ff, afB, GA, biv, Ph, en, anW, g, bz, awi, dr, LB, eM, wK, mx, cj, gZ, bix, XO, it); #c1(DSP) c2(NP_004406) c3(desmoplakin isoform 1) c4(146154, 146155) c5(146156, 146157, 146158, 146159, 146160, 146161) c6( )c7(aOR, T, Ke, by, n, aml, ci, aFJ, biy, XM, cd, Hr, ip, biA, av, cc, biu, biw, bit, ey, lQ, Tj, af, biz, Y, ff, afB, GA, biv, Ph, en, anW, g, bz, awi, dr, LB, eM, wK, mx, cj, gZ, bix, XO, it); #c1(DSPP) c2(NP_055023) c3(dentin sialophosphoprotein preproprotein) c4(178341) c5(178342, 178343, 178344, 178345, 178346, 178347, 178348, 178349, 178350, 178351) c6( )c7(biD, T, Rz, oe, biB, n, bic, Ry, Pz, biy, V, ad, Hr, cc, awb, biw, ae, af, Y, ff, en, dr, wK, mG, gZ, biE, eo, ano, awa); #c1(DST) c2(NP_001714) c3(dystonin isoform 1e precursor) c4(120453) c5(120454, 120455, 120456, 120457, 120458, 120459) c6( )c7(T, arM, bcb, biF, Pt, aFa, bO, biH, bS, Jz, ct, ci, fu, bX, V, XM, que, ad, RD, eW, aqJ, aT, aU, lQ, ae, il, aW, Y, ff, en, g, Z, t, pm, aRJ, amt, sS, cA, Jr, biG, ZT, Ei, dy, m, XO, X); #c1(DST) c2(NP_056363) c3(dystonin isoform 1eA precursor) c4(187312, 187313) c5(187314, 187315, 187316, 187317, 187318, 187319) c6( )c7(T, arM, bcb, biF, Pt, aFa, bO, biH, bS, Jz, ct, ci, fu, bX, V, XM, aue, ad, RD, eW, aqJ, aT, aU, lQ, ae, il, aW, Y, ff, en, g, Z, t, pm, aRJ, amt, sS, cA, Jr, biG, ZT, Ei, dy, m, XO, X); #c1(DST) c2(NP_899236) c3(dystonin isoform 1) c4(258061) c5(258062, 258063, 258064, 258065, 258066, 258067) c6( )c7(T, arM, bcb, biF, Pt, aFa, bO, biH, bS, Jz, ct, ci, fu, bX, V, XM, aue, ad, RD, eW, aqJ, aT, aU, lL, ae, il, aW, Y, ff, en, g, Z, t, pm, aRJ, amt, sS, cA, Jr, biG, ZT, Ei, dy, m, XO, X); #c1(DST) c2(XP_005249367) c3(dystonin isoform X1) c4( ) c5(269240, 269241, 269242, 269243, 269244) c6( )c7(T, arM, bcb, biF, Pt, aFa, bO, biH, bS, Jz, ct, ci, fu, bX, V, XM, aue, ad, RD, eW, aqJ, aT, aU, lQ, ae, il, aW, Y, ff, en, g, Z, t, pm, aRJ, amt, sS, cA, Jr, biG, ZT, Ei, dy, m, XO, X); #c1(DST) c2(XP_005249372) c3(dystonin isoform X3) c4(269245) c5(269246, 269247, 269248, 269249, 269250, 269251) c6( )c7(T, arM, bcb, biF, Pt, aFa, bO, biH, bS, Jz, ct, ci, fu, bX, V, XM, aue, ad, RD, eW, aqJ, aT, aU, lQ, ae, il, aW, Y, ff, en, g, Z, t, pm, aRJ, amt, sS, cA, Jr, biG, ZT, Ei, dy, m, XO, X); #c1(DST) c2(XP_005249380) c3 (dystonin isoform X9) c4(269252, 269253) c5(269254, 269255, 269256, 269257, 269258, 269259) c6( )c7(T, arM, bcb, biF, Pt, aFa, bO, biH, bS, Jz, ct, ci, fu, bX, V, XM, que, ad, RD, eW, aqJ, aT, aU, lQ, ae, il, aW, Y, ff, en, g, Z, t, pm, aRJ, amt, sS, cA, Jr, biG, ZT, Ei, dy, m, XO, X); #c1(DST) c2(XP_005249381) c3(dystonin isoform X10) c4(269260, 269261) c5(269262, 269263, 269264, 269265, 269266, 269267, 269268, 269269, 269270, 269271, 269272, 269273) c6( )c7(T, arM, bcb, biF, Pt, aFa, bO, biH, bS, Jz, ct, ci, fu, bX, V, XM, aue, ad, RD, eW, aqJ, aT, aU, lQ, ae, il, aW, Y, ff, en, g, Z, t, pm, aRJ, amt, sS, cA, Jr, biG, ZT, Ei, dy, m, XO, X); #c1(DST) c2(XP_011513127) c3(dystonin isoform X11) c4( )c5(304932) c6( )c7(T, arM, bcb, biF, Pt, aFa, bO, biH, bS, Jz, ct, ci, fu, bX, V, XM, aue, ad, RD, eW, aqJ, aT, aU, lQ, ae, il, aW, Y, ff, en, g, Z, t, pm, aRJ, amt, sS, cA, Jr, biG, ZT, Ei, dy, m, XO, X); #c1(DST) c2(XP_011513128) c3(dystonin isoform X12) c4(304933) c5(304934, 304935, 304936, 304937, 304938, 304939, 304940, 304941, 304942, 304943, 304944, 304945) c6( )c7(T, arM, bcb, biF, Pt, aFa, bO, biH, bS, Jz, ct, ci, fu, bX, V, XM, aue, ad, RD, eW, aqJ, aT, aU, lQ, ae, il, aW, Y, ff, en, g, Z, t, pm, aRJ, amt, sS, cA, Jr, biG, ZT, Ei, dy, m, XO, X); #c1(DSTN) c2(XP_011527445) c3(destrin isoform X1) c4(322199, 322200) c5(322201, 322202, 322203, 322204, 322205, 322206, 322207) c6( )c7(T, d, aE, bE, bB, By, dL, m, K, as, eA, X); #c1(DSTN) c2(XP_011527446) c3(destrin isoform X2) c4( )c5(322208, 322209, 322210, 322211, 322212) c6( )c7(T, d, aE, bE, bB, By, dL, m, K, as, eA, X); #c1(DSTYK) c2(NP_056190) c3(dual serine/threonine and tyrosine protein kinase isoform 1) c4(186213) c5(186214, 186215, 186216, 186217, 186218, 186219) c6( )c7(bil); #c1(DSTYK) c2(NP_955749) c3(dual serine/threonine and tyrosine protein kinase isoform 2) c4(261925, 261926) c5(261927, 261928, 261929, 261930, 261931, 261932, 261933, 261934, 261935, 261936, 261937, 261938) c6( )c7(bil); #c1(DSTYK) c2(XP_011507694) c3(dual serine/threonine and tyrosine protein kinase isoform X1) c4(297698) c5( )c6( )c7(bil); #c1(DTD1) c2(NP_543010) c3(D-tyrosyl-tRNA(Tyr) deacylase 1) c4(233229) c5(233230, 233231, 233232, 233233, 233234, 233235) c6( )c7(ale); #c1(DTHD1) c2(NP_001130008) c3(death domain-containing protein 1 isoform 2) c4( )c5(52413, 52414, 52415, 52416, 52417, 52418) c6( )c7(V, nx, j); #c1(DTHD1) c2(NP_001164171) c3(death domain-containing protein 1 isoform 1) c4( )c5(66715, 66716, 66717, 66718, 66719) c6( )c7(V, nx, j); #c1(DTHD1) c2(XP_006714077) c3(death domain-containing protein 1 isoform X2) c4(287538, 287539) c5(287540) c6( )c7(V, nx, j); #c1(DTL) c2(NP_001273158) c3(denticleless protein homolog isoform 2) c4( )c5(104387, 104388, 104389, 104390, 104391, 104392, 104393) c6( )c7(T, ht, aA, bB, q, qK, aT, dz, ey, et, ff, r, nf, aF, en, t, KM, ac, bE, eD, m, B); #c1(DTL) c2(NP_001273159) c3(denticleless protein homolog isoform 3) c4(104394) c5(104395, 104396, 104397, 104398, 104399, 104400) c6( ) c7(T, ht, aA, bB, q, qK, aT, dz, ey, et, ff, r, nf, af, en, t, KM, ac, bE, eD, m, B); #c1(DTL) c2(NP_057532) c3(denticleless protein homolog isoform 1) c4(191275) c5(191276, 191277, 191278, 191279, 191280, 191281) c6( )c7(T, ht, aA, bB, q, qK, aT, dz, ey, et, ff, r, nf, aF, en, t, KM, ac, bE, eD, m, B); #c1(DTL) c2(XP_011507916) c3(denticleless protein homolog isoform X1) c4(298225) c5( )c6( )c7(T, ht, aA, bB, q, qK, aT, dz, ey, et, ff, r, nf, aF, en, t, KM, ac, bE, eD, m, B); #c1(DTNA) c2(NP_001121647) c3(dystrobrevin alpha isoform 9) c4(46010) c5(46011, 46012, 46013, 46014, 46015, 46016, 46017, 46018, 46019) c6( )c7(no, Fh, xs, oj, biJ, j, Bu, bfW); #c1(DTNA) c2(NP_001185867) c3(dystrobrevin alpha isoform 10) c4(76601) c5(76602, 76603, 76604, 76605, 76606, 76607) c6( )c7(no, Fh, xs, oj, biJ, j, Bu, bfW); #c1(DTNA) c2(NP_001185868) c3(dystrobrevin alpha isoform (l) c4(76608) c5(76609, 76610, 76611, 76612, 76613, 76614) c6( )c7(no, Fh, xs, oj, biJ, j, Bu, bfW); #c1(DTNA) c2(NP_001185871) c3(dystrobrevin alpha isoform 14) c4( )c5(76615, 76616, 76617, 76618, 76619) c6( )c7(no, Fh, xs, oj, biJ, j, Bu, bfW); #c1(DTNA) c2(NP_001185873) c3(dystrobrevin alpha isoform 16) c4( )c5(76620, 76621, 76622, 76623, 76624) c6( )c7(no, Fh, xs, oj, biJ, j, Bu, bfW); #c1(DTNA) c2(NP_001381) c3(dystrobrevin alpha isoform 1) c4(117470, 117471, 117472) c5(117473, 117474, 117475, 117476, 117477, 117478, 117479, 117480, 117481, 117482, 117483, 117484, 117485, 117486, 117487, 117488, 117489, 117490, 117491, 117492, 117493, 117494) c6( )c7(no, Fh, xs, oj, biJ, j, Bu, bfW); #c1(DTNA) c2(NP_001382) c3(dystrobrevin alpha isoform 3) c4(117495) c5(117496, 117497, 117498, 117499, 117500, 117501) c6( )c7(no, Fh, xs, oj, biJ, j, Bu, bfW); #c1(DTNA) c2(NP_001383) c3(dystrobrevin alpha isoform 7) c4(117502) c5(117503, 117504, 117505, 117506, 117507, 117508) c6( )c7(no, Fh, xs, oj, biJ, j, Bu, bfW); #c1(DTNA) c2(NP_116757) c3(dystrobrevin alpha isoform 2) c4(226404, 226405) c5(226406, 226407, 226408, 226409, 226410, 226411, 226412, 226413, 226414, 226415, 226416, 226417) c6( )c7(no, Fh, xs, oj, biJ, j, Bu, bfW); #c1(DTNA) c2(NP_116760) c3(dystrobrevin alpha isoform 4) c4(226432) c5(226433, 226434, 226435, 226436, 226437, 226438) c6( )c7(no, Fh, xs, oj, biJ, j, Bu, bfW); #c1(DTNA) c2(NP_116762) c3(dystrobrevin alpha isoform 6) c4( )c5(226439, 226440, 226441, 226442, 226443, 226444, 226445, 226446) c6( ) c7(no, Fh, xs, oj, biJ, j, Bu, bfW); #c1(DTNA) c2(XP_005258273) c3(dystrobrevin alpha isoform X17) c4(275601) c5(275602) c6( )c7(no, Fh, xs, oj, biJ, j, Bu, bfW); #c1(DTNA) c2(XP_005258278) c3(dystrobrevin alpha isoform X24) c4(275603) c5(275604) c6( )c7(no, Fh, xs, oj, biJ, j, Bu, bfW); #c1(DTNA) c2(XP_005258279) c3(dystrobrevin alpha isoform X25) c4(275605) c5(275606, 275607) c6( )c7(no, Fh, xs, oj, biJ, j, Bu, bfW); #c1(DTNB) c2(NP_001243232) c3(dystrobrevin beta isoform 6) c4(90200) c5(90201, 90202, 90203, 90204, 90205, 90206) c6( )c7(cc, j, fd); #c1(DTNB) c2(NP_001243233) c3(dystrobrevin beta isoform 7) c4(90207) c5(90208) c6( )c7(cc, j, fd); #c1(DTNB) c2(NP_001243237) c3(dystrobrevin beta isoform 8) c4( )c5(90224, 90225, 90226, 90227, 90228, 90229) c6( )c7(cc, j, fd); #c1(DTNB) c2(NP_068707) c3(dystrobrevin beta isoform 1) c4(210075, 210076) c5(210077, 210078, 210079, 210080, 210081, 210082, 210083) c6( )c7(cc, j, fd); #c1(DTNB) c2(NP_149159) c3(dystrobrevin beta isoform 2) c4(227408, 227409, 227410) c5(227411, 227412, 227413, 227414, 227415, 227416, 227417, 227418, 227419, 227420, 227421, 227422) c6( )c7(cc, j, fd); #c1(DTNB) c2(NP_149160) c3(dystrobrevin beta isoform 3) c4(227423) c5(227424, 227425, 227426, 227427, 227428, 227429) c6( )c7(cc, j, fd); #c1(DTNB) c2(NP_899204) c3(dystrobrevin beta isoform 4) c4(258019) c5(258020, 258021, 258022, 258023, 258024, 258025) c6( )c7(cc, j, fd); #c1(DTNB) c2(NP_899205) c3(dystrobrevin beta isoform 5) c4(258026) c5(258027, 258028, 258029, 258030, 258031, 258032) c6( ) c7(cc, j, fd); #c1(DTNB) c2(XP_005264241) c3(dystrobrevin beta isoform X9) c4(278712) c5(278713, 278714, 278715, 278716, 278717, 278718) c6( )c7(cc, j, fd); #c1(DTNB) c2(XP_005264243) c3(dystrobrevin beta isoform X15) c4( )c5(278719, 278720, 278721, 278722, 278723, 278724, 278725, 278726, 278727, 278728, 278729, 278730, 278731, 278732, 278733, 278734, 278735) c6( )c7(cc, j, fd); #c1(DTNB) c2(XP_011530973) c3(dystrobrevin beta isoform X2) c4(326932) c5( )c6( )c7(cc, j, fd); #c1(DTNB) c2(XP_011530983) c3(dystrobrevin beta isoform X14) c4(326933) c5(326934, 326935, 326936, 326937, 326938) c6( )c7(cc, j, fd); #c1(DTNBP1) c2(NP_001258596) c3(dysbindin isoform C) c4( )c5(96173, 96174, 96175, 96176, 96177) c6( )c7(iH, dA, oe, bO, mS, bS, ct, bdr, mW, Fo, bk, oi, mY, mV, he, na, KC, ml, lE, gl, Eg, Z, cm, jb, mZ, bik, mX, hq, ac, cA, XF, lF, sv, bdq, lB, xg, mT); #c1(DTNBP1) c2(NP_001258597) c3(dysbindin isoform d) c4(96178, 96179, 96180) c5(96181, 96182, 96183, 96184, 96185, 96186, 96187, 96188, 96189, 96190, 96191) c6( ) c7(iH, dA, oe, bO, mS, bS, ct, bdr, mW, Fo, bK, oi, mY, mV, he, na, KC, mU, lE, gl, Eg, Z, cm, jb, mZ, bik, mX, hq, ac, CA, XF, lF, sv, bdq, lB, xg, mT); #c1(DTNBP1) c2(NP_115498) c3(dysbindin isoform a) c4(222930, 222931) c5(222932, 222933, 222934, 222935, 222936, 222937, 222938, 222939, 222940, 222941, 222942, 222943, 222944, 222945) c6( )c7(iH, dA, oe, bO, mS, bS, ct, bdr, mW, Fo, bK, oi, mY, mV, he, na, KC, mU, lE, gl, Eg, Z, cm, jb, mZ, bik, mX, hq, ac, cA, XF, lF, sv, bdq, lB, xg, mT); #c1(DTNBP1) c2(NP_898861) c3(dysbindin isoform b) c4(257776, 257777) c5(257778, 257779, 257780, 257781, 257782, 257783, 257784, 257785, 257786, 257787, 257788, 257789) c6( ) c7(iH, dA, oe, bO, mS, bS, ct, bdr, mW, Fo, bK, oi, mY, mV, he, na, KC, mU, lE, gl, Eg, Z, cm, jb, mZ, bik, mX, hq, ac, CA, XF, lF, sv, bdq, lB, xg, mT); #c1(DTX1) c2(XP_011536311) c3(E3 ubiquitin-protein ligase DTX1 isoform X1) c4(333665) c5(333666, 333667, 333668, 333669, 333670, 333671) c6( )c7(d, yS, eF, eH, apk, h, il, Fc, eA, BA, At); #c1(DTX2) c2(NP_001096064) c3(probable E3 ubiquitin-protein ligase DTX2 isoform a) c4(40997, 40998, 40999, 41000, 41001, 41002, 41003) c5(41004, 41005, 41006, 41007, 41008, 41009, 41010, 41011, 41012, 41013, 41014, 41015, 41016, 41017, 41018, 41019, 41020, 41021, 41022, 41023, 41024, 41025, 41026, 41027, 41028, 41029, 41030, 41031, 41032, 41033, 41034, 41035, 41036, 41037) c6( )c7(dB); #c1(DTX2) c2(NP_001096066) c3(probable E3 ubiquitin-protein ligase DTX2 isoform b) c4(41038, 41039) c5(41040, 41041, 41042, 41043, 41044, 41045, 41046, 41047, 41048, 41049) c6( )c7(dB); #c1(DTX2) c2(XP_011514079) c3(probable E3 ubiquitin-protein ligase DTX2 isoform X3) c4(306646, 306647, 306648) c5(306649, 306650, 306651, 306652, 306653, 306654, 306655, 306656, 306657, 306658, 306659, 306660, 306661, 306662, 306663, 306664, 306665, 306666, 306667, 306668, 306669, 306670, 306671, 306672, 306673, 306674, 306675, 306676, 306677, 306678, 306679, 306680, 306681, 306682, 306683, 306684, 306685, 306686, 306687, 306688, 306689, 306690) c6( )c7(dB); #c1(DTX3) c2(NP_001273174) c3(probable E3 ubiquitin-protein ligase DTX3 isoform b) c4(104478, 104479) c5(104480, 104481, 104482, 104483, 104484, 104485, 104486, 104487, 104488, 104489, 104490, 104491) c6( )c7(aT); #c1(DTX3) c2(NP_001273175) c3(probable E3 ubiquitin-protein ligase DTX3 isoform a) c4(104492, 104493) c5(104494, 104495, 104496, 104497, 104498, 104499, 104500, 104501, 104502, 104503, 104504, 104505, 104506, 104507, 104508, 104509, 104510, 104511, 104512, 104513) c6( )c7(aT); #c1(DTX4) c2(NP_001287656) c3(E3 ubiquitin-protein ligase DTX4 isoform 2) c4(112442) c5(112443, 112444, 112445, 112446, 112447) c6( )c7(qK, BA); #c1(DTX4) c2(NP_055992) c3(E3 ubiquitin-protein ligase DTX4 isoform 1) c4(184994) c5(184995, 184996, 184997, 184998, 184999, 185000, 185001) c6( )c7(qK, BA); #c1(DTYMK) c2(NP_036277) c3(thymidylate kinase isoform 1) c4(172833) c5(172834, 172835, 172836, 172837, 172838, 172839) c6( ) c7(eW, T, jp, bE, eH, bB, h, bS, eF, ae, X, ad); #c1(DUOX1) c2(NP_787954) c3(dual oxidase 1 precursor) c4(252151, 252152) c5(252153, 252154, 252155, 252156, 252157, 252158, 252159, 252160, 252161, 252162, 252163, 252164, 252165, 252166, 252167, 252168, 252169, 252170, 252171, 252172) c6( )c7(T, al, cz, cR, n, aj, V, cd, J, ad, av, M, h, cy, K, ae, aly, wC, d, Cu, g, bz, ck, qJ, eB, at, Da, B, bil); #c1(DUOX1) c2(XP_011519983) c3(dual oxidase 1 isoform X1) c4(314829) c5(314830, 314831, 314832, 314833) c6( )c7(T, al, cz, cR, n, aj, V, cd, J, ad, av, M, h, cy, K, ae, aly, wC, d, Cu, g, bz, ck, qJ, eB, at, Da, B, bil); #c1(DUOX1) c2(XP_011519984) c3(dual oxidase 1 isoform X2) c4(314834) c5(314835, 314836, 314837, 314838, 314839, 314840) c6( )c7(T, al, cz, cR, n, aj, V, cd, J, ad, av, M, h, cy, K, ae, aQy, wC, d, Cu, g, bz, ck, qJ, eB, at, Da, B, bil); #c1(DUOX2) c2(NP_054799) c3(dual oxidase 2 precursor) c4(178005) c5(178006, 178007, 178008, 178009, 178010, 178011) c6( )c7(al, cz, cR, n, aj, V, bil, cd, J, biN, Vp, av, hd, RN, M, h, cy, ji, K, wC, azU, A, biM, Cu, iV, bz, ck, qJ, eB, at, Da, gT, B); #c1(DUOXA1) c2(NP_001263193) c3(dual oxidase maturation factor 1 isoform 1) c4(96992) c5(96993, 96994, 96995, 96996, 96997, 96998, 96999, 97000, 97001, 97002, 97003, 97004, 97005) c6( )c7(T, d, aW, aT, h, biP, bz, eA, aQy); #c1(DUOXA1) c2(NP_001263194) c3(dual oxidase maturation factor 1 isoform 2) c4(97006) c5(97007, 97008, 97009, 97010, 97011, 97012) c6( )c7(T, d, aW, aT, h, biP, bz, eA, aQy); #c1(DUOXA1) c2(XP_005254830) c3(dual oxidase maturation factor 1 isoform X1) c4(273330) c5(273331, 273332, 273333, 273334, 273335) c6( )c7(T, d, aW, aT, h, biP, bz, eA, aQy); #c1(DUOXA1) c2(XP_006720815) c3(dual oxidase maturation factor 1 isoform X3) c4(293693) c5(293694, 293695, 293696, 293697, 293698, 293699, 293700, 293701) c6( )c7(T, d, aW, aT, h, biP, bz, eA, aQy); #c1(DUOXA1) c2(XP_006720817) c3(dual oxidase maturation factor 1 isoform X2) c4(293702, 293703, 293704) c5(293705, 293706, 293707, 293708, 293709, 293710, 293711, 293712, 293713, 293714, 293715, 293716, 293717, 293718, 293719, 293720, 293721, 293722, 293723) c6( )c7(T, d, aW, aT, h, biP, bz, eA, aQy); #c1(DUOXA1) c2(XP_011520487) c3(dual oxidase maturation factor 1 isoform X5) c4(315374) c5( )c6( )c7(T, d, aW, aT, h, biP, bz, eA, aQy); #c1(DUOXA1) c2(XP_011520488) c3(dual oxidase maturation factor 1 isoform X6) c4(315375, 315376) c5(315377, 315378, 315379, 315380, 315381, 315382, 315383, 315384, 315385, 315386, 315387, 315388, 315389, 315390) c6( )c7(T, d, aW, aT, h, biP, bz, eA, aQy); #c1(DUOXA2) c2(NP_997464) c3(dual oxidase maturation factor 2) c4(265542) c5(265543, 265544, 265545, 265546, 265547, 265548) c6( )c7(RN, biR, ji, iV, biP, bil, bz); #c1(DUPD1) c2(NP_001003892) c3(dual specificity phosphatase DUPD1) c4(20243) c5(20244, 20245, 20246, 20247, 20248, 20249) c6( )c7(W); #c1(DUS2) c2(NP_001258691) c3(tRNA-dihydrouridine(20) synthase [NAD(P)+]-like isoform 1) c4(96350) c5(96351, 96352, 96353, 96354, 96355, 96356, 96357, 96358, 96359, 96360) c6( )c7(bz, eM); #c1(DUS2) c2(NP_001258692) c3(tRNA-dihydrouridine(20) synthase [NAD(P)+]-like isoform 2) c4(96361) c5(96362, 96363, 96364, 96365, 96366, 96367) c6( )c7(bz, eM); #c1(DUSP1) c2(NP_004408) c3(dual specificity protein phosphatase 1) c4(146162, 146163, 146164) c5(146165, 146166, 146167, 146168, 146169, 146170, 146171, 146172, 146173, 146174, 146175, 146176, 146177, 146178, 146179, 146180, 146181, 146182) c6( )c7(bM, T, dD, aC, id, ea, uG, bO, aA, cz, jh, bS, j, ct, C, ci, v, eq, V, fr, q, Pm, cd, ad, zo, dP, Bl, av, biS, aE, db, gd, aT, h, cy, K, bq, MO, ae, A, aW, ff, eH, cr, r, eZ, aq, lo, vs, kp, en, cl, Z, t, g, bz, eF, iK, ck, ar, ou, rR, nM, eM, iT, hL, cj, aGN, wR, ME, m, y, bt, B); #c1(DUSP10) c2(NP_009138) c3(dual specificity protein phosphatase 10) c4(171019, 171020, 171021) c5(171022, 171023, 171024, 171025, 171026, 171027, 171028, 171029, 171030, 171031, 171032, 171033) c6( )c7(bM, ar, aik, oz, r, hp, K, EA, ey, act, q, ad); #c1(DUSP10) c2(XP_011507418) c3(dual specificity protein phosphatase 10 isoform X2) c4( )c5(297328, 297329, 297330, 297331, 297332, 297333, 297334, 297335, 297336, 297337) c6( ) c7(bM, ar, aik, oz, r, hp, K, EA, ey, act, q, ad); #c1(DUSP11) c2(NP_003575) c3(RNA/RNP complex-1-interacting phosphatase) c4(139291, 139292, 139293) c5(139294, 139295, 139296, 139297, 139298, 139299, 139300, 139301, 139302, 139303, 139304, 139305, 139306, 139307, 139308, 139309, 139310, 139311) c6( )c7(bE); #c1(DUSP12) c2(NP_009171) c3(dual specificity protein phosphatase (2) c4(171242) c5(171243, 171244, 171245, 171246, 171247, 171248) c6( )c7(bL, T, aE, eM, iT, iU, NT, hL, ik, cm, lo, v, K, m, C, t, bt, bz, c1); #c1(DUSP13) c2(NP_001007272) c3(dual specificity protein phosphatase 13 isoform 1) c4(22664) c5(22665, 22666, 22667, 22668, 22669) c6( ) c7(cm, eR, bX); #c1(DUSP13) c2(NP_001007273) c3(dual specificity protein phosphatase 13 isoform 2) c4(22670, 22671) c5(22672, 22673, 22674, 22675, 22676, 22677) c6( )c7(cm, eR, bX); #c1(DUSP13) c2(NP_001007274) c3(dual specificity protein phosphatase 13 isoform 3 precursor) c4(22678) c5(22679, 22680, 22681, 22682, 22683, 22684) c6( )c7(cm, eR, bX); #c1(DUSP13) c2(NP_057448) c3(dual specificity protein phosphatase 13 isoform 6) c4( )c5(190937, 190938, 190939, 190940, 190941, 190942, 190943, 190944, 190945) c6( )c7(cm, eR, bX); #c1(DUSP13) c2(XP_005269940) c3(dual specificity protein phosphatase 13 isoform X3) c4( )c5(283008, 283009) c6( )c7(cm, eR, bX); #c1(DUSP13) c2(XP_011538155) c3(dual specificity protein phosphatase 13 isoform X5) c4(335786) c5(335787, 335788, 335789, 335790, 335791, 335792) c6( )c7(cm, eR, bX); #c1(DUSP14) c2(NP_008957) c3(dual specificity protein phosphatase 14) c4(169791) c5(169792, 169793, 169794, 169795, 169796, 169797, 169798, 169799, 169800, 169801) c6( )c7(Y); #c1(DUSP15) c2(NP_542178) c3(dual specificity protein phosphatase 15 isoform a) c4(232213, 232214, 232215, 232216) c5(232217, 232218, 232219, 232220, 232221, 232222, 232223, 232224, 232225, 232226, 232227, 232228, 232229, 232230, 232231, 232232, 232233, 232234, 232235, 232236, 232237, 232238, 232239, 232240) c6( )c7(V, eo); #c1(DUSP15) c2(NP_817130) c3(dual specificity protein phosphatase 15 isoform b) c4( )c5(253136, 253137, 253138, 253139, 253140, 253141, 253142, 253143, 253144, 253145, 253146) c6( )c7(V, eo); #c1(DUSP16) c2(XP_005253545) c3(dual specificity protein phosphatase 16 isoform X2) c4( )c5(272550) c6( )c7(ck, vs, ae, ad); #c1(DUSP16) c2(XP_006719218) c3(dual specificity protein phosphatase 16 isoform X1) c4(292316, 292317, 292318) c5(292319, 292320, 292321, 292322, 292323, 292324, 292325, 292326, 292327, 292328, 292329, 292330, 292331, 292332, 292333, 292334, 292335) c6( )c7(ck, vs, ae, ad); #c1(DUSP18) c2(NP_001291723) c3(dual specificity protein phosphatase 18 isoform a) c4(116389, (16390) c5(116391, 116392, 116393, 116394, 116395, 116396, 116397, 116398, 116399, 116400, 116401, 116402, 116403, 116404, 116405, 116406, 116407, 116408, 116409, 116410, 116411, 116412, 116413, 116414) c6( )c7(ac); #c1(DUSP18) c2(NP_001291725) c3(dual specificity protein phosphatase 18 isoform b) c4( )c5(116415, 116416, 116417, 116418) c6( )c7(ac); #c1(DUSP2) c2(NP_004409) c3(dual specificity protein phosphatase 2) c4(146183) c5(146184, 146185, 146186, 146187, 146188, 146189) c6( )c7(ul, iW, NT, fd, bB, Fb, uu, hF, Fc, eA, ad, dP, pX, aT, bK, K, he, ae, aW, kp, cm, bz, ck, bP, bE, Ei, m, pk); #c1(DUSP21) c2(NP_071359) c3(dual specificity protein phosphatase 21) c4(211198, 211199) c5(211200, 211201, 211202, 211203, 211204, 211205, 211206, 211207, 211208, 211209, 211210, 211211) c6( )c7(B); #c1(DUSP22) c2(NP_001273484) c3(dual specificity protein phosphatase 22 isoform a) c4(105003) c5( )c6( )c7(aW, hp, aT); #c1(DUSP22) c2(NP_064570) c3(dual specificity protein phosphatase 22 isoform b) c4(203034) c5(203035, 203036, 203037, 203038, 203039, 203040) c6( )c7(aW, hp, aT); #c1(DUSP22) c2(XP_011513059) c3(dual specificity protein phosphatase 22 isoform X1) c4( )c5(304739, 304740, 304741, 304742, 304743, 304744) c6( )c7(aW, hp, aT); #c1(DUSP23) c2(NP_060293) c3(dual specificity protein phosphatase 23) c4(195223) c5(195224, 195225, 195226, 195227, 195228, 195229, 195230, 195231, 195232, 195233, 195234, 195235, 195236, 195237) c6( )c7(cm, bW, bX); #c1(DUSP27) c2(XP_005245660) c3(inactive dual specificity phosphatase 27 isoform X1) c4(266854) c5(266855, 266856, 266857, 266858, 266859, 266860, 266861, 266862, 266863, 266864, 266865, 266866, 266867, 266868) c6( )c7(aWm, an, ac, cz); #c1(DUSP28) c2(NP_001028747) c3(dual specificity phosphatase 28) c4(30603) c5(30604, 30605, 30606, 30607, 30608, 30609, 30610, 30611, 30612, 30613) c6( )c7(cm, eA, T, d, K); #c1(DUSP3) c2(NP_004081) c3(dual specificity protein phosphatase 3) c4(143400, 143401) c5(143402, 143403, 143404, 143405, 143406, 143407, 143408, 143409, 143410, 143411, 143412, 143413) c6( )c7(T, eM, ci, m, t, ae, ad); #c1(DUSP4) c2(NP_001385) c3(dual specificity protein phosphatase 4 isoform 1) c4(117509) c5(117510, 117511, 117512, 117513, 117514, 117515) c6( )c7(iu, T, cc, d, aW, qy, aT, r, n, ci, K, bz, eA, t, ik, fq, q); #c1(DUSP4) c2(XP_011542730) c3(dual specificity protein phosphatase 4 isoform X1) c4(341308) c5(341309, 341310, 341311, 341312, 341313, 341314) c6( )c7(iu, T, cc, d, aW, qy, aT, r, n, ci, K, bz, eA, t, ik, fq, q); #c1(DUSP5) c2(NP_004410) c3(dual specificity protein phosphatase 5) c4(146190) c5(146191, 146192, 146193, 146194, 146195, 146196) c6( )c7(T, aFG, aW, jh, cj, aT, aA, h, aF, ho, t, Cd, Wt); #c1(DUSP6) c2(NP_001937) c3(dual specificity protein phosphatase 6 isoform a) c4(122334) c5(122335, 122336, 122337, 122338, 122339, 122340) c6( )c7(bL, T, aC, wL, v, j, ci, zP, AD, biT, Yn, hd, aE, aT, bK, K, eu, zQ, d, s, qt, aW, aq, g, cl, LV, t, bz, cm, ar, bill, nM, cA, nB, AM, Qu, eM, hL, m, it, B); #c1(DUSP6) c2(NP_073143) c3(dual specificity protein phosphatase 6 isoform b) c4(212791, 212792) c5(212793, 212794, 212795, 212796, 212797, 212798, 212799, 212800, 212801, 212802, 212803, 212804) c6( )c7(bL, T, aC, wL, v, j, ci, zP, AO, biT, Yn, hd, aE, aT, bK, K, eu, zQ, d, s, qt, aW, aq, g, cl, LV, t, bz, cm, ar, bill, nM, cA, nB, AM, Qu, eM, hL, m, it, B); #c1(DUSP7) c2(NP_001938) c3(dual specificity protein phosphatase 7) c4(122341, 122342) c5(122343, 122344, 122345, 122346, 122347, 122348, 122349, 122350, 122351, 122352, 122353, 122354) c6( )c7(ck, x, T, iT, k, hF, bt); #c1(DUSP8) c2(XP_011518235) c3(dual specificity protein phosphatase 8 isoform X1) c4(312267) c5(312268, 312269, 312270, 312271, 312272, 312273, 312274, 312275, 312276, 312277) c6( )c7(fd, cj); #c1(DUSP9) c2(XP_011529425) c3(dual specificity protein phosphatase 9 isoform X2) c4(324852) c5(324853, 324854) c6( )c7(bL, py, n, aj, v, ei, J); #c1(DUT) c2(NP_001020419) c3(deoxyuridine 5′-triphosphate nucleotidohydrolase, mitochondrial isoform 1 precursor) c4(28767, 28768, 28769) c5(28770, 28771, 28772, 28773, 28774, 28775, 28776, 28777, 28778, 28779, 28780, 28781) c6( )c7(eW, T, eM, atC, aw, cr, jh, biV, ci, as, B); #c1(DUT) c2(NP_001020420) c3(deoxyuridine 5′-triphosphate nucleotidohydrolase, mitochondrial isoform 3) c4( )c5(28782, 28783, 28784, 28785, 28786) c6( ) c7(eW, T, eM, atC, aw, cr, jh, biV, ci, as, B); #c1(DUT) c2(NP_001939) c3(deoxyuridine 5′-triphosphate nucleotidohydrolase, mitochondrial isoform 2) c4(122355) c5(122356, 122357, 122358, 122359, 122360, 122361, 122362) c6( )c7(eW, T, eM, atC, aw, cr, jh, biV, ci, as, B); #c1(DUX4) c2(XP_011529816) c3(double homeobox protein 4 isoform X2) c4( )c5(325627) c6( )c7(li, bv, QG, xs, oj, lo, rx, bl, K, aiG, Bu, dx); #c1(DVL2) c2(NP_004413) c3(segment polarity protein dishevelled homolog DVL-2) c4(146197) c5(146198, 146199, 146200, 146201, 146202, 146203, 146204, 146205) c6(146206, 146207) c7(jf, av, d, aW, aT, r, ci, eA, Z, alo, q, De); #c1(DVL3) c2(NP_004414) c3(segment polarity protein dishevelled homolog DVL-3) c4(146208, 146209, 146210) c5( )c6( )c7(av, T, eM, ff, it, at, aln, g, en, as, bz, B, aa); #c1(DVL3) c2(XP_011510815) c3(segment polarity protein dishevelled homolog DVL-3 isoform X2) c4( )c5(301261, 301262, 301263, 301264, 301265, 301266, 301267, 301268, 301269, 301270, 301271, 301272, 301273, 301274, 301275, 301276, 301277, 301278, 301279) c6( )c7(av, T, eM, ff, it, at, aln, g, en, as, bz, B, aa); #c1(DXO) c2(XP_006715068) c3(decapping and exoribonuclease protein isoform X2) c4(288574) c5(288575, 288576, 288577, 288578, 288579, 288580, 288581, 288582, 288583, 288584, 288585, 288586, 288587, 288588, 288589, 288590, 288591, 288592, 288593, 288594, 288595, 288596, 288597, 288598, 288599, 288600, 288601, 288602, 288603, 288604, 288605, 288606, 288607, 288608, 288609, 288610, 288611, 288612, 288613, 288614, 288615, 288616, 288617, 288618, 288619, 288620, 288621, 288622, 288623, 288624, 288625, 288626) c6( )c7(bN, A, W, cH, eZ, cj, v, nA, ei, bD, biW, af, all); #c1(DXO) c2(XP_006725751) c3(decapping and exoribonuclease protein isoform X2) c4(296965) c5(296966, 296967, 296968, 296969, 296970, 296971, 296972, 296973, 296974, 296975, 296976, 296977, 296978, 296979) c6( )c7(bN, A, W, cH, eZ, cj, v, nA, ei, bD, biW, af, all); #c1(DYM) c2(NP_060123) c3(dymeclin) c4(194274, 194275) c5(194276, 194277, 194278, 194279, 194280, 194281, 194282, 194283, 194284, 194285, 194286, 194287, 194288) c6( )c7(ID, bM, T, dD, ea, aO, e, if, aw, l, aj, eq, C, nA, bd, qN, q, J, rc, BY, sO, Ot, lC, aRD, K, lz, fb, rv, aa, ly, lx, kT, gZ, aNL, aS, yA, vF, aZG); #c1(DYM) c2(XP_006722554) c3(dymeclin isoform X10) c4(294733) c5(294734, 294735, 294736, 294737, 294738) c6( )c7(ID, bM, T, dD, ea, aO, e, if, aw, l, aj, eq, C, nA, bd, qN, q, J, rc, BY, sO, Ot, lC, aRD, K, lz, fb, rv, aa, ly, lx, kT, gZ, aNL, aS, yA, vF, aZG); #c1(DYNAP) c2(NP_775900) c3(dynactin-associated protein) c4(250619) c5(250620, 250621, 250622, 250623, 250624, 250625) c6( )c7(T); #c1(DYNC1H1) c2(NP_001367) c3(cytoplasmic dynein 1 heavy chain 1) c4(117350) c5(117351, 117352, 117353, 117354, 117355, 117356) c6( )c7(T, Ol, zN, dG, Vd, fu, nA, biY, cF, DK, nv, kf, bix, jy, bl, bU, Oq, PK, FD, Pg, QK, biZ, aNB, c, ql, mf, gv, XO, WI, B); #c1(DYNC1H1) c2(NP_001129029) c3(cytoplasmic dynein 1 intermediate chain 1 isoform c) c4(50677) c5(50678, 50679, 50680, 50681, 50682, 50683) c6( )c7(aDV, bX); #c1(DYNC1H1) c2(NP_001265351) c3(cytoplasmic dynein 1 intermediate chain 1 isoform e) c4(98353, 98354) c5(98355, 98356, 98357, 98358, 98359, 98360, 98361, 98362, 98363, 98364, 98365, 98366) c6( )c7(aOV, bX); #c1(DYNC1H1) c2(XP_011514163) c3(cytoplasmic dynein 1 intermediate chain 1 isoform X1) c4(306743) c5(306744, 306745, 306746, 306747, 306748, 306749) c6( )c7(aOV, bX); #c1(DYNC111) c2(XP_011514164) c3(cytoplasmic dynein 1 intermediate chain 1 isoform X2) c4(306750, 306751) c5(306752, 306753, 306754, 306755, 306756, 306757, 306758, 306759, 306760, 306761, 306762, 306763, 306764, 306765, 306766, 306767) c6( )c7(aDV, bX); #c1(DYNC112) c2(NP_001258715) c3(cytoplasmic dynein 1 intermediate chain 2 isoform 2) c4(96411) c5(96412, 96413, 96414, 96415, 96416, 96417) c6( )c7(T): #c1(DYNC112) c2(NP_001258718) c3(cytoplasmic dynein 1 intermediate chain 2 isoform 3) c4(96418) c5(96419, 96420, 96421, 96422, 96423, 96424, 96425, 96426, 96427, 96428) c6( )c7(T); #c1(DYNC112) c2(NP_001258719) c3(cytoplasmic dynein 1 intermediate chain 2 isoform 4) c4(96429) c5(96430, 96431, 96432, 96433, 96434, 96435) c6( )c7(T); #c1(DYNC112) c2(NP_001369) c3(cytoplasmic dynein I intermediate chain 2 isoform 1) c4(117366, 117367, 117368) c5(117369, 117370, 117371, 117372, 117373, 117374, 117375, 117376, 117377, 117378, 117379, 117380, 117381, 117382, 117383, 117384, 117385, 117386, 117387, 117388, 117389, 117390) c6( )c7(T); #c1(DYNC112) c2(XP_005246424) c3(cytoplasmic dynein 1 intermediate chain 2 isoform X4) c4(267089) c5(267090, 267091, 267092, 267093, 267094, 267095, 267096, 267097, 267098, 267099) c6( )c7(T); #c1(DYNC1L11) c2(NP_057225) c3(cytoplasmic dynein 1 light intermediate chain 1) c4(189574) c5(189575, 189576, 189577, 189578, 189579, 189580) c6( )c7(ds); #c1(DYNC2H1) c2(NP_001073932) c3(cytoplasmic dynein 2 heavy chain 1 isoform 2) c4(36567) c5(36568, 36569, 36570, 36571, 36572, 36573) c6( )c7(ft, bja, bjc, bjb, bjd, j, zG, eA, af); #c1(DYNC2H1) c2(NP_001368) c3(cytoplasmic dynein 2 heavy chain 1 isoform 1) c4(117357, 117358, 117359) c5(117360, 117361, 117362, 117363, 117364, 117365) c6( )c7(ft, bja, bjc, bjb, bjd, j, zG, eA, af); #c1(DYNC2H1) c2(XP_006718966) c3(cytoplasmic dynein 2 heavy chain 1 isoform X1) c4( )c5(292138, 292139, 292140, 292141, 292142, 292143, 292144, 292145, 292146, 292147, 292148, 292149) c6( )c7(ft, bja, bjc, bjb, bjd, j, zG, eA, af); #c1(DYNLL1) c2(NP_001032584) c3(dynein light chain 1, cytoplasmic) c4(31358, 31359) c5(31360, 31361, 31362, 31363, 31364, 31365, 31366, 31367, 31368, 31369, 31370, 31371, 31372, 31373, 31374, 31375, 31376, 31377, 31378, 31379, 31380, 31381, 31382, 31383, 31384, 31385, 31386, 31387) c6( )c7(T, abY, iW, WT, jh, aA, bB, n, fd, Fb, fr, ci, ad, ip, hX, av, aAF, aT, io, K, hB, ae, eu, jf, aW, aEl, ff, aq, kp, aF, en, g, iR, t, cm, ik, eM, bE, adg, B); #c1(DYNLL2) c2(NP_542408) c3(dynein light chain 2, cytoplasmic) c4(232401) c5(232402, 232403, 232404, 232405, 232406, 232407) c6( )c7(tb, Y, QU, yP, hL, ww, K, cl, B, hq); #c1(DYNLRB1) c2(NP_001268658) c3(dynein light chain roadblock-type 1 isoform c) c4( )c5(100091) c6( )c7(T, ft, eM, aA, cU, bz); #c1(DYNLRB1) c2(NP_054902) c3(dynein light chain roadblock-type 1 isoform a) c4(178266, 178267) c5(178268, 178269, 178270, 178271, 178272, 178273, 178274, 178275, 178276, 178277, 178278) c6( )c7(T, ft, eM, aA, cU, bz); #c1(DYNLRB1) c2(XP_005260625) c3(dynein light chain roadblock-type 1 isoform X1) c4(276747) c5(276748, 276749, 276750, 276751, 276752, 276753) c6( )c7(T, ft, eM, aA, cl, bz); #c1(DYNLT1) c2(NP_001278531) c3(dynein light chain Tctex-type 1 isoform 2) c4( )c5(109793, 109794, 109795, 109796, 109797) c6( ) c7(ID, bu, BY); #c1(DYNLT1) c2(NP_001278532) c3(dynein light chain Tctex-type 1 isoform 3) c4( )c5(109798, 109799, 109800, 109801, 109802, 109803) c6( )c7(ID, bu, BY); #c1(DYNLT1) c2(NP_006510) c3(dynein light chain Tctex-type 1 isoform 1) c4(165890, 165891, 165892) c5(165893, 165894, 165895, 165896, 165897, 165898) c6( )c7(ID, bu, BY); #c1(DYNLT3) c2(NP_006511) c3(dynein light chain Tctex-type 3) c4(165899, 165900, 165901) c5(165902, 165903, 165904, 165905, 165906, 165907, 165908, 165909, 165910, 165911, 165912, 165913, 165914, 165915, 165916, 165917, 165918, 165919) c6( )c7(afR, HU, ayj, nC, Be); #c1(DYRK1A) c2(NP_001387) c3(dual specificity tyrosine-phosphorylation-regulated kinase 1A isoform 1) c4(117516, 117517) c5(117518, 117519, 117520, 117521, 117522, 117523) c6( )c7(T, Kk, ha, bjf, bS, ct, nA, fK, cd, eA, anj, Hx, zf, HC, h, iY, K, he, af, ak, d, Y, bje, Z, QK, f, pH, EW, bb, pa, Kr, agl, U, R, bD, xg); #c1(DYRK1A) c2(NP_567824) c3(dual specificity tyrosine-phosphorylation-regulated kinase 1A isoform 3) c4(233561) c5(233562, 233563, 233564, 233565, 233566, 233567, 233568, 233569, 233570, 233571, 233572, 233573, 233574, 233575, 233576, 233577) c6( )c7(T, Kk, ha, bjf, bS, ct, nA, fK, cd, eA, anj, Hx, zf, HC, h, iY, K, he, af, ak, d, Y, bje, Z, QK, f, pH, EW, bb, pa, Kr, agl, U, R, bD, xg); #c1(DYRK1A) c2(NP_569120) c3(dual specificity tyrosine-phosphorylation-regulated kinase 1A isoform 2) c4(233671) c5(233672, 233673, 233674, 233675, 233676, 233677, 233678, 233679, 233680, 233681, 233682, 233683) c6( )c7(T, Kk, ha, bjf, bS, ct, nA, fK, cd, eA, anj, Hx, zf, HC, h, iY, K, he, af, ak, d, Y, bje, Z, QK, f, pH, EW, bb, pa, Kr, agl, U, R, bD, xg); #c1(DYRK1A) c2(NP_569122) c3(dual specificity tyrosine-phosphorylation-regulated kinase IA isoform 5) c4(233684, 233685, 233686) c5(233687, 233688, 233689, 233690, 233691, 233692, 233693, 233694, 233695, 233696, 233697, 233698, 233699, 233700, 233701, 233702, 233703, 233704) c6( )c7(T, Kk, ha, bjf, bS, ct, nA, fK, cd, eA, anj, Hx, zf, HC, h, iY, K, he, af, ak, d, Y, bje, Z, QK, f, pH, EW, bb, pa, Kr, agl, U, R, bD, xg); #c1(DYRK1A) c2(XP_011527786) c3(dual specificity tyrosine-phosphorylation-regulated kinase 1A isoform X4) c4(322755) c5( )c6( ) c7(T, Kk, ha, bjf, bS, ct, nA, fK, cd, eA, anj, Hx, zf, HC, h, iY, K, he, af, ak, d, Y, bje, Z, QK, f, pH, EW, bb, pa, Kr, agl, U, R, bD, xg); #c1(DYRK1B) c2(NP_004705) c3(dual specificity tyrosine-phosphorylation-regulated kinase (B isoform a) c4(148961) c5(148962, 148963, 148964, 148965, 148966) c6( )c7(bv, T, aE, eM, bE, eH, bB, kp, m, ei, g, eF, t, bz); #c1(DYRK1B) c2(NP_006474) c3(dual specificity tyrosine-phosphorylation-regulated kinase (B isoform b) c4(165477) c5(165478, 165479, 165480, 165481, 165482) c6( )c7(bv, T, aE, eM, bE, eH, bB, kp, m, ei, g, eF, t, bz); #c1(DYRK1B) c2(NP_006475) c3(dual specificity tyrosine-phosphorylation-regulated kinase 1B isoform c) c4(165483) c5(165484, 165485, 165486, 165487, 165488) c6( )c7(bv, T, aE, eM, bE, eH, bB, kp, m, ei, g, eF, t, bz); #c1(DYRK1B) c2(XP_005259452) c3(dual specificity tyrosine-phosphorylation-regulated kinase 18 isoform X1) c4( )c5(276111) c6( )c7(bv, T, aE, eM, bE, eH, bB, kp, m, ei, g, eF, t, bz); #c1(DYRK1B) c2(XP_011525771) c3(dual specificity tyrosine-phosphorylation-regulated kinase 1B isoform X2) c4( )c5(320173) c6( )c7(bv, T, aE, eM, bE, eH, bB, kp, m, ei, g, eF, t, bz); #c1(DYRK1B) c2(XP_011525772) c3(dual specificity tyrosine-phosphorylation-regulated kinase IB isoform X3) c4( )c5(320174) c6( )c7(bv, T, aE, eM, bE, CH, bB, kp, m, ei, g, eF, t, bz); #c1(DYRK2) c2(NP_003574) c3(dual specificity tyrosine-phosphorylation-regulated kinase 2 isoform 1) c4( )c5(139281, 139282, 139283, 139284, 139285, 139286, 139287, 139288, 139289, 139290) c6( )c7(T, iE, aW, hj, aA, als, aT, aF, ey, g, cm); #c1(DYRK2) c2(NP_006473) c3(dual specificity tyrosine-phosphorylation-regulated kinase 2 isoform 2) c4(165459, 165460, 165461, 165462) c5(165463, 165464, 165465, 165466, 165467, 165468, 165469, 165470, 165471, 165472, 165473, 165474, 165475, 165476) c6( )c7(T, iE, aW, hj, aA, aHs, aT, aF, ey, g, cm); #c1(DYRK3) c2(NP_001004023) c3(dual specificity tyrosine-phosphorylation-regulated kinase 3 isoform b) c4(20359, 20360) c5(20361, 20362, 20363, 20364, 20365, 20366, 20367, 20368, 20369, 20370, 20371, 20372, 20373, 20374, 20375, 20376, 20377, 20378, 20379) c6( )c7(mA, K, bjg); #c1(DYRK3) c2(NP_003573) c3(dual specificity tyrosine-phosphorylation-regulated kinase 3 isoform a) c4(139274) c5(139275, 139276, 139277, 139278, 139279, 139280) c6( )c7(mA, K, bjg); #c1(DYSF) c2(NP_001123927) c3(dysferlin isoform 2) c4(48018) c5(48019, 48020, 48021, 48022, 48023, 48024, 48025, 48026, 48027, 48028, 48029, 48030, 48031, 48032, 48033, 48034, 48035, 48036, 48037, 48038, 48039, 48040, 48041, 48042, 48043, 48044, 48045, 48046, 48047, 48048, 48049, 48050, 48051, 48052, 48053, 48054, 48055, 48056, 48057, 48058, 48059, 48060, 48061, 48062, 48063, 48064, 48065, 48066, 48067, 48068, 48069, 48070, 48071, 48072, 48073, 48074, 48075, 48076, 48077, 48078, 48079, 48080, 48081, 48082, 48083, 48084, 48085, 48086, 48087, 48088, 48089, 48090, 48091, 48092, 48093, 48094, 48095, 48096, 48097, 48098, 48099, 48100, 48101, 48102, 48103, 48104, 48105, 48106, 48107, 48108, 48109, 48110, 48111, 48112, 48113, 48114, 48115) c6(48116, 48117, 48118) c7(aBD, e, V, Rk, ad, cc, no, Ae, Ws, bjh, oj, Ak, bl, K, H, alm, aqZ, xs, lb, Ad, bji, adu, aBH, U, km, oh, R, ul, atO); #c1(DYSF) c2(NP_001124448) c3(dysferlin isoform 9) c4( )c5(48949) c6( )c7(aBD, e, V, Rk, ad, cc, no, Ae, Ws, bjh, oj, Ak, bl, K, H, aTm, aqZ, xs, lb, Ad, bji, adu, aBH, U, km, oh, R, ul, atO); #c1(DYSF) c2(NP_001124449) c3(dysferlin isoform 10) c4( )c5(48950) c6( )c7(aBD, e, V, Rk, ad, cc, no, Ae, Ws, bjh, oj, Ak, bl, K, H, aTm, aqZ, xs, lb, Ad, bji, adu, aBH, U, km, oh, R, ul, atO); #c1(DYSF) c2(NP_001124450) c3(dysferlin isoform 11) c4(48951) c5(48952, 48953, 48954, 48955, 48956, 48957, 48958, 48959, 48960, 48961) c6(48962, 48963) c7(aBD, e, V, Rk, ad, cc, no, Ae, Ws, bjh, oj, Ak, bl, K, H, aTm, aqZ, xs, lb, Ad, bji, adu, aBH, U, km, oh, R, ul, atO); #c1(DYSF) c2(NP_001124451) c3(dysferlin isoform 12) c4(48964) c5(48965, 48966, 48967, 48968, 48969, 48970, 48971, 48972, 48973, 48974) c6(48975, 48976) c7(aBD, e, V, Rk, ad, cc, no, Ae, Ws, bjh, oj, Ak, bl, K, H, aTm, aqZ, xs, lb, Ad, bji, adu, aBH, U, km, oh, R, ul, atO); #c1(DYSF) c2(NP_001124452) c3(dysferlin isoform 13) c4(48977) c5(48978, 48979, 48980, 48981, 48982, 48983, 48984, 48985, 48986, 48987) c6(48988, 48989) c7(aBD, e, V, Rk, ad, cc, no, Ae, Ws, bjh, oj, Ak, bl, K, H, aTm, aqZ, xs, lb, Ad, bji, adu, aBH, U, km, oh, R, ul, atO); #c1(DYSF) c2(NP_001124453) c3(dysferlin isoform 14) c4(48990) c5(48991, 48992, 48993, 48994, 48995, 48996, 48997, 48998, 48999, 49000) c6(49001, 49002) c7(aBD, e, V, Rk, ad, cc, no, Ae, Ws, bjh, oj, Ak, bl, K, H, aTm, aqZ, xs, lb, Ad, bji, adu, aBH, U, km, oh, R, ul, atO); #c1(DYSF) c2(NP_001124454) c3(dysferlin isoform 7) c4(49003) c5(49004, 49005, 49006, 49007, 49008, 49009, 49010, 49011, 49012, 49013) c6(49014, 49015) c7(aBD, e, V, Rk, ad, cc, no, Ae, Ws, bjh, oj, Ak, bl, K, H, aTm, aqZ, xs, lb, Ad, bji, adu, aBH, U, km, oh, R, ul, atO); #c1(DYSF) c2(NP_001124455) c3(dysferlin isoform 6) c4(49016) c5(49017, 49018, 49019, 49020, 49021, 49022, 49023, 49024, 49025, 49026) c6(49027, 49028) c7(aBD, e, V, Rk, ad, cc, no, Ae, Ws, bjh, oj, Ak, bl, K, H, aTm, aqZ, xs, lb, Ad, bji, adu, aBH, U, km, oh, R, uL, atO); #c1(DYSF) c2(NP_001124456) c3(dysferlin isoform 5) c4(49029) c5(49030, 49031, 49032, 49033, 49034, 49035, 49036, 49037, 49038, 49039) c6(49040, 49041) c7(aBD, e, V, Rk, ad, cc, no, Ae, Ws, bjh, oj, Ak, bl, K, H, aTm, aqZ, xs, lb, Ad, bji, adu, aBH, U, km, oh, R, ul, at); #c1(DYSF) c2(NP_001124457) c3(dysferlin isoform 4) c4(49042) c5(49043, 49044, 49045, 49046, 49047, 49048, 49049, 49050, 49051, 49052) c6(49053, 49054) c7(aBD, e, V, Rk, ad, cc, no, Ae, Ws, bjh, oj, Ak, bl, K, H, aTm, aqZ, xs, lb, Ad, bji, adu, aBH, U, km, oh, R, ul, atO); #c1(DYSF) c2(NP_001124458) c3(dysferlin isoform 3) c4( )c5(49055) c6( )c7(aBD, e, V, Rk, ad, cc, no, AB, Ws, bjh, oj, Ak, bl, K, H, alm, aqZ, xs, lb, Ad, bji, adu, aBH, U, km, oh, R, ul, atO); #c1(DYSF) c2(NP_001124459) c3(dysferlin isoform 1) c4(49056) c5(49057, 49058, 49059, 49060, 49061, 49062, 49063, 49064, 49065, 49066) c6(49067, 49068) c7(aBD, e, V, Rk, ad, cc, no, Ae, Ws, bjh, oj, Ak, bl, K, H, aTm, aqZ, xs, lb, Ad, bji, adu, aBH, U, km, oh, R, ul, atO); #c1(DYSF) c2(NP_003485) c3(dysferlin isoform 8) c4(138713) c5(138714, 138715, 138716, 138717, 138718, 138719, 138720, 138721, 138722, 138723) c6(138724, 138725) c7(aBD, e, V, Rk, ad, cc, no, Ae, Ws, bjh, oj, Ak, bl, K, H, alm, aqZ, xs, lb, Ad, bji, adu, aBH, U, km, oh, R, ul, atO); #c1(DYX1C1) c2(NP_001028731) c3(dyslexia susceptibility 1 candidate gene I protein isoform b) c4(30553) c5(30554, 30555, 30556, 30557, 30558, 30559, 30560, 30561, 30562, 30563, 30564, 30565, 30566, 30567, 30568, 30569) c6( )c7(T, gU, aW, aT, r, at, yA, alP, cm, bj, q, aR, bN, MG); #c1(DYX1C1) c2(NP_001028732) c3 (dyslexia susceptibility 1 candidate gene 1 protein isoform c) c4(30570, 30571) c5(30572, 30573, 30574, 30575, 30576, 30577, 30578, 30579, 30580, 30581) c6( )c7(T, gU, aW, aT, r, at, yA, alP, cm, bj, q, aR, bN, MG); #c1(DYX1C1) c2(NP_570722) c3(dyslexia susceptibility 1 candidate gene 1 protein isoform a) c4(234274) c5(234275, 234276, 234277, 234278, 234279, 234280) c6( ) c7(T, gU, aW, aT, r, at, yA, alP, cm, bj, q, aR, bN, MG); #c1(DZIP1) c2(NP_055749) c3(zinc finger protein DZIP1 isoform 1) c4(183266) c5(183267, 183268, 183269, 183270, 183271, 183272, 183273, 183274, 183275, 183276) c6( )c7(aW, aT); #c1(DZIP1) c2(NP_945319) c3(zinc finger protein DZIP1 isoform 2) c4(260800) c5(260801, 260802, 260803, 260804, 260805, 260806, 260807, 260808, 260809, 260810) c6( )c7(aW, aT); #c1(EZF1) c2(NP_005216) c3(transcription factor E2F1) c4(154097) c5(154098, 154099, 154100, 154101, 154102, 154103, 154104) c6( )c7(T, fR, if, al, fd, bB, ci, aE, aEX, K, aNZ, hu, pk, eL, cp, eG, yo, anw, aW, az, uY, bjk, g, eF, cm, bw, qJ, BM, nB, dc, cj, QF, m, Xu, iu, lT, aBT, aw, ht, fN, n, fr, qN, br, co, ft, h, aPe, hn, dL, Go, A, kj, aiB, PX, aq, kp, YE, bP, dB, hk, hR, it, AN, B, AS, jx, aA, q, eA, hW, nv, aT, cy, an, ae, af, jf, xR, Y, r, en, bjl, t, iK, ck, rR, Dy, np, bt, id, iW, jh, bs, bS, hF, ad, hX, av, py, fb, uX, d, lR, qt, ff, xs, eH, dq, aF, iR, bz, dR, es, gk, bE, bC, X); #c1(E2F3) c2(NP_001230005) c3(transcription factor E2F3 isoform 2) c4(85759) c5(85760, 85761, 85762, 85763, 85764, 85765) c6( )c7(T, id, eX, oH, ci, q, er, ad, aHR, av, iu, aT, eV, dL, ae, eL, dx, d, iU, aW, Y, ff, en, g, t, cm, bz, es, bP, hR, m, bC, bt, B); #c1(E2F3) c2(NP_001940) c3(transcription factor E2F3 isoform 1) c4(122363) c5(122364, 122365, 122366, 122367, 122368, 122369) c6( )c7(T, id, eX, OH, ci, q, er, ad, aHR, av, iu, aT, eV, dL, ae, eL, dx, d, iU, aW, Y, ff, en, g, t, cm, bz, es, bP, hR, m, bC, bt, B); #c1(E2F3) c2(XP_011512628) c3 (transcription factor E2F3 isoform X3) c4(303593) c5(303594, 303595, 303596, 303597, 303598, 303599) c6( )c7(T, id, eX, oH, ci, q, er, ad, aHR, av, iu, aT, eV, dl, ae, eL, dx, d, il, aW, Y, ff, en, g, t, cm, bz, es, bP, hR, m, bC, bt, B); #c1(E2F4) c2(NP_001941) c3(transcription factor E2F4) c4(122370) c5(122371, 122372, 122373, 122374, 122375, 122376) c6( )c7(T, aw, aA, bB, j, ci, q, ad, av, hd, bjn, kH, aT, K, pk, ae, B, kj, je, aW, r, aF, iV, bjm, t, iM, nB, aBU, bE, gX, m, bt, X); #c1(E2F5) c2(NP_001077057) c3(transcription factor E2F5 isoform 2) c4(37622) c5(37623, 37624, 37625, 37626, 37627, 37628) c6( )c7(aW, t, T, aT); #c1(E2F5) c2(NP_001942) c3 (transcription factor E2F5 isoform 1) c4(122377, 122378, 122379, 122380) c5(122381, 122382, 122383, 122384, 122385, 122386, 122387, 122388, 122389, 122390, 122391, 122392, 122393) c6( )c7(aW, t, T, aT); #c1(E2F5) c2(XP_006716497) c3(transcription factor E2F5 isoform X1) c4( )c5(290359, 290360, 290361, 290362, 290363, 290364, 290365, 290366, 290367, 290368, 290369, 290370) c6( )c7(aW, t, T, aT); #c1(E2F6) c2(NP_001265204) c3(transcription factor E2F6 isoform 2) c4(97932, 97933) c5(97934, 97935, 97936, 97937, 97938, 97939, 97940, 97941) c6( )c7(cT, eM, jh, eF, t, B); #c1(E2F6) c2(NP_001265207) c3(transcription factor E2F6 isoform 3) c4( )c5(97942, 97943, 97944, 97945, 97946, 97947, 97948, 97949) c6( )c7(cT, eM, jh, eF, t, B); #c1(E2F6) c2(NP_937987) c3(transcription factor E2F6 isoform 1) c4(259457, 259458) c5(259459, 259460, 259461, 259462, 259463, 259464) c6( )c7(cT, eM, jh, eF, t, B); #c1(E2F6) c2(XP_011508619) c3(transcription factor E2F6 isoform X1) c4( )c5(299237, 299238, 299239, 299240, 299241, 299242) c6( )c7(cT, eM, jh, eF, t, B); #c1(E2F7) c2(NP_976328) c3(transcription factor E2F7) c4(263156, 263157) c5(263158, 263159, 263160, 263161, 263162, 263163, 263164, 263165, 263166, 263167, 263168, 263169) c6( )c7(W, BN, ne, hb, oe, ff, cj, hy, en, t, bt, B); #c1(E2F7) c2(XP_011536270) c3(transcription factor E2F7 isoform X3) c4(333605, 333606) c5(333607, 333608, 333609, 333610, 333611, 333612) c6( )c7(W, BN, ne, hb, oe, ff, cj, hy, en, t, bt, B); #c1(E2F7) c2(XP_011536271) c3(transcription factor E2F7 isoform X4) c4( )c5(333613, 333614, 333615, 333616, 333617, 333618) c6( )c7(W, BN, ne, hb, oe, ff, cj, hy, en, t, bt, B); #c1(E2F8) c2(NP_078956) c3(transcription factor E2F8) c4(216249, 216250) c5(216251, 216252, 216253, 216254, 216255, 216256, 216257, 216258, 216259, 216260, 216261, 216262, 216263, 216264, 216265, 216266) c6( )c7(t, T, B, m); #c1(E4F1) c2(NP_004415) c3(transcription factor E4F1 isoform 1) c4(146211) c5(146212, 146213, 146214, 146215, 146216, 146217) c6( )c7(bt, K, iT); #c1(EAF1) c2(NP_149074) c3(ELL-associated factor 1) c4(227013) c5(227014, 227015, 227016, 227017, 227018, 227019) c6( )c7(ck); #c1(EAF1) c2(XP_011532468) c3(ELL-associated factor 1 isoform X1) c4(328620) c5(328621, 328622, 328623, 328624, 328625, 328626) c6( )c7(ck); #c1(EAF2) c2(NP_060926) c3(ELL-associated factor 2) c4(198714, 198715) c5(198716, 198717, 198718, 198719, 198720, 198721, 198722, 198723, 198724, 198725, 198726, 198727) c6( )c7(av, bjo, T, fM, iu, aDZ, bt, ae, B, ad, hq); #c1(EARS2) c2(NP_001077083) c3(probable glutamate-tRNA ligase, mitochondrial) c4(37697) c5(37698, 37699, 37700, 37701, 37702, 37703, 37704, 37705, 37706, 37707) c6( )c7(dD, ea, bjp, eZ, hD, j, eq); #c1(EBAG9) c2(NP_001265867) c3(receptor-binding cancer antigen expressed on SiSo cells) c4(99513, 99514, 99515, 99516) c5(99517, 99518, 99519, 99520, 99521, 99522, 99523, 99524, 99525, 99526, 99527, 99528, 99529, 99530, 99531, 99532, 99533, 99534, 99535, 99536, 99537, 99538, 99539, 99540, 99541, 99542, 99543, 99544) c6( )c7(T, aC, aA, Wb, fr, ci, AD, eA, ad, dP, jz, ib, aT, dL, ae, bc, JI, aW, ff, aF, en, cl, g, t, uC, bz, ck, AM, mg, hL, m, jM, y, B); #c1(EBF1) c2(NP_076870) c3(transcription factor COE1 isoform 2) c4(214193) c5(214194, 214195, 214196, 214197, 214198, 214199, 214200, 214201, 214202, 214203) c6( )c7(ck, x, T, ac, eX, jh, n, eV, j, k, Ea, cm, V, Hr); #c1(EBF1) c2(NP_874367) c3(transcription factor COE1 isoform 3) c4(256636) c5(256637, 256638, 256639, 256640, 256641, 256642) c6( )c7(ck, x, T, ac, eX, jh, n, eV, j, k, Ea, cm, V, Hr); #c1(EBF2) c2(NP_073150) c3(transcription factor COE2) c4(212815, 212816) c5(212817, 212818, 212819, 212820, 212821, 212822, 212823, 212824, 212825, 212826, 212827, 212828) c6( )c7(iU, eF, eH, j, bX, bV, fq, yn); #c1(EBF3) c2(NP_001005463) c3(transcription factor COE3) c4(21685) c5(21686, 21687, 21688, 21689, 21690, 21691) c6( )c7(jf, T, dB, ff, yg, aA, af, Ea, en, af); #c1(EBF3) c2(XP_005252725) c3(transcription factor COE3 isoform X4) c4(272032) c5(272033, 272034, 272035, 272036, 272037, 272038) c6( )c7(jf, T, dB, ff, yg, aA, aF, Ea, en, af); #c1(EBF3) c2(XP_011537876) c3(transcription factor COE3 isoform X9) c4(335376) c5( )c6( )c7(jf, T, dB, ff, yg, aA, aF, Ea, en, af); #c1(EBF3) c2(XP_011537877) c3(transcription factor COE3 isoform X11) c4( )c5(335377, 335378, 335379, 335380, 335381, 335382) c6( )c7(jf, T, dB, ff, yg, aA, aF, Ea, en, af); #c1(EBF4) c2(NP_001103984) c3(transcription factor COE4) c4(42056, 42057) c5(42058, 42059, 42060, 42061, 42062, 42063, 42064, 42065, 42066, 42067, 42068, 42069) c6( )c7(y); #c1(EBF4) c2(XP_005260828) c3(transcription factor COE4 isoform X2) c4( )c5(276931, 276932, 276933, 276934, 276935, 276936) c6( )c7(y); #c1(EBF4) c2(XP_006723663) c3(transcription factor COE4 isoform X1) c4(295283) c5( )c6( )c7(y); #c1(EBI3) c2(NP_005746) c3(interleukin-27 subunit beta precursor) c4(158974, 158975, (58976) c5(158977, 158978, 158979, 158980, 158981, 158982) c6( )c7(bL, bM, T, dD, aAf, fM, dA, Dw, aEj, ea, AL, jh, aA, n, lD, pl, eq, V, ip, hX, x, aE, Bj, ds, dV, aT, qU, h, dz, JK, pq, il, ly, A, d, mR, aAu, aW, Y, zb, ano, oV, eZ, fe, af, CO, jg, iV, eE, iK, bz, DF, lx, ck, cT, eM, iT, rK, k, y, bt, cn); #c1(EBP) c2(NP_006570) c3(3-beta-hydroxysteroid-Delta(8), Delta(7)-isomerase) c4(166294, 166295, 166296) c5(166297, 166298, 166299, 166300, 166301, 166302, 166303, 166304, 166305, 166306, 166307, 166308, 166309, 166310, 166311, 166312, 166313, 166314) c6( )c7(bL, gx, T, bM, jh, cz, bB, v, afE, beW, q, br, ad, hX, x, ke, hd, ahj, dV, aT, h, jy, hn, dL, K, aAt, aVg, bl, jj, nJ, ae, eL, eG, d, bex, il, aW, Y, oV, fc, r, aq, aF, Pd, cm, bw, bz, At, qa, ek, QA, ck, nM, bjq, iT, bE, aVh, k, arx, cP, y, bt, B, ao); #c1(EBPL) c2(NP_001265565) c3(emopamil-binding protein-like isoform 2) c4(98846, 98847) c5(98848, 98849, 98850, 98851, 98852, 98853, 98854, 98855, 98856, 98857, 98858, 98859, 98860, 98861, 98862, 98863) c6( )c7(agd, alC, sO, RX, abi, Pg, Ps, bK, aan, lw, af, agZ); #c1(EBPL) c2(NP_115954) c3(emopamil-binding protein-like isoform 1) c4(224910, 224911) c5(224912, 224913, 224914, 224915, 224916, 224917, 224918, 224919) c6( )c7(agd, alC, sO, RX, abi, Pg, Ps, bK, aan, lw, af, agZ); #c1(ECD) c2(NP_001129224) c3 (protein SGT1 isoform 2) c4(51258) c5(51259, 51260, 51261, 51262, 51263, 51264) c6( )c7(Pw, T, hM, aW, aA, hD, aT, aF, cd); #c1(ECD) c2(NP_001129225) c3(protein SGT1 isoform 3) c4(51265, 51266) c5(51267, 51268, 51269, 51270, 51271, 51272, 51273, 51274, 51275, 51276, 51277, 51278) c6( ) c7(Pw, T, hM, aW, aA, hD, aT, aF, cd); #c1(ECD) c2(NP_009196) c3(protein SGT1 isoform 1) c4(171437, 171438) c5(171439, 171440, 171441, 171442, 171443, 171444, 171445, 171446, 171447, 171448, 171449, 171450, 171451) c6( )c7(Pw, T, hM, aW, aA, hD, aT, aF, cd); #c1(ECE2) c2(NP_001032401) c3(endothelin-converting enzyme 2 isoform B) c4(31296) c5(31297, 31298, 31299, 31300, 31301, 31302) c6( )c7(ea, V, dD, eq, qa); #c1(ECE2) c2(NP_001093590) c3(endothelin-converting enzyme 2 isoform D) c4(40318) c5(40319, 40320, 40321, 40322, 40323, 40324) c6( )c7(ea, V, dD, eq, qa); #c1(ECE2) c2(NP_001093591) c3(endothelin-converting enzyme 2 isoform E) c4(40325) c5(40326, 40327, 40328, 40329, 40330, 40331) c6( )c7(ea, V, dD, eq, qa); #c1(ECE2) c2(NP_055508) c3(endothelin-converting enzyme 2 isoform A) c4(181614) c5(181615, 181616, 181617, 181618, 181619, 181620) c6( )c7(ea, V, dD, eq, qa); #c1(ECE2) c2(NP_115707) c3(endothelin-converting enzyme 2 isoform C) c4(223662) c5(223663, 223664, 223665, 223666, 223667, 223668) c6( )c7(ea, V, dD, eq, qa); #c1(ECEL1) c2(NP_001277716) c3(endothelin-converting enzyme-like 1 isoform 2) c4(109395) c5(109396, 109397, 109398, 109399, 109400, 109401) c6( )c7(bjs, bjr, bjt, zW, cm, A1); #c1(ECEL1) c2(NP_004817) c3(endothelin-converting enzyme-like 1 isoform 1) c4(150026) c5(150027, 150028, 150029, 150030, 150031, 150032) c6( )c7(bjs, bjr, bjt, zW, cm, A1); #c1(ECHDC1) c2(NP_001099014) c3(ethylmalonyl-CoA decarboxylase isoform 3) c4( )c5(41662, 41663, 41664, 41665, 41666) c6( )c7(aW, t, Y, aT); #c1(ECHDC1) c2(NP_001099015) c3 (ethylmalonyl-CoA decarboxylase isoform 4) c4( )c5(41667, 41668, 41669, 41670, 41671) c6( )c7(aW, t, Y, aT); #c1(ECHDC1) c2(NP_001132982) c3(ethylmalonyl-CoA decarboxylase isoform 5) c4(52752, 52753, 52754, 52755, 52756) c5(52757, 52758, 52759, 52760, 52761, 52762, 52763, 52764, 52765, 52766, 52767, 52768, 52769, 52770, 52771, 52772, 52773) c6( )c7(aW, t, Y, aT); #c1(ECHDC1) c2(XP_005267105) c3(ethylmalonyl-CoA decarboxylase isoform X1) c4(280705) c5(280706, 280707, 280708, 280709, 280710, 280711, 280712, 280713, 280714, 280715, 280716, 280717, 280718, 280719, 280720, 280721, 280722, 280723, 280724, 280725, 280726, 280727, 280728, 280729, 280730, 280731, 280732) c6( )c7(aW, t, Y, aT); #c1(ECHDC1) c2(XP_005267106) c3(ethylmalonyl-CoA decarboxylase isoform X4) c4(280733, 280734, 280735, 280736) c5( )c6( )c7(aW, t, Y, aT); #c1(ECHDC1) c2(XP_005267107) c3(ethylmalonyl-CoA decarboxylase isoform X2) c4( )c5(280737, 280738, 280739, 280740, 280741, 280742, 280743, 280744, 280745, 280746, 280747, 280748, 280749, 280750, 280751, 280752, 280753, 280754) c6( )c7(aW, t, Y, aT); #c1(ECHDC1) c2(XP_011534245) c3(ethylmalonyl-CoA decarboxylase isoform X5) c4(330602) c5(330603, 330604, 330605, 330606, 330607, 330608) c6( )c7(aW, t, Y, aT); #c1(ECHDC3) c2(NP_078969) c3(enoyl-CoA hydratase domain-containing protein 3, mitochondrial precursor) c4(216326, 216327, 216328) c5(216329, 216330, 216331, 216332, 216333, 216334, 216335, 216336, 216337, 216338, 216339, 216340, 216341, 216342, 216343, 216344, 216345, 216346) c6( )c7(aw, ac); #c1(ECHS1) c2(NP_004083) c3(enoyl-CoA hydratase, mitochondrial) c4(143414) c5(143415, 143416, 143417, 143418, 143419, 143420) c6( )c7(mD, d, aW, aA, aT, v, hE, B, ajU, hX); #c1(ECI1) c2(NP_001171500) c3(enoyl-CoA delta isomerase 1, mitochondrial isoform 2 precursor) c4(70319) c5(70320, 70321, 70322, 70323, 70324, 70325) c6( )c7(T, mo); #c1(ECI1) c2(NP_001910) c3(enoyl-CoA delta isomerase 1, mitochondrial isoform 1 precursor) c4(122140) c5(122141, 122142, 122143, 122144, 122145, 122146) c6( )c7(T, mo); #c1(ECI2) c2(NP_006108) c3(enoyl-CoA delta isomerase 2, mitochondrial isoform 1) c4(161908) c5(161909, 161910, 161911, 161912, 161913, 161914, 161915, 161916, 161917, 161918, 161919, 161920, 161921, 161922, 161923, 161924) c6( )c7(yd, bM, ck, il, iT, dW, bw, fh, cd); #c1(ECI2) c2(NP_996667) c3(enoyl-CoA delta isomerase 2, mitochondrial isoform 2) c4(264347, 264348) c5(264349, 264350, 264351, 264352, 264353, 264354, 264355, 264356, 264357, 264358) c6( )c7(yd, bM, ck, iQ, iT, dW, bw, fh, cd); #c1(ECSCR) c2(NP_001071161) c3(endothelial cell-specific chemotaxis regulator isoform 1 precursor) c4(35339) c5(35340, 35341, 35342, 35343, 35344, 35345) c6( )c7(bw, Jn, eM); #c1(ECSCR) c2(NP_001280668) c3(endothelial cell-specific chemotaxis regulator isoform 2 precursor) c4(111573) c5(111574, 111575, 111576, 111577, 111578, 111579) c6( ) c7(bw, Jn, eM); #c1(ECSIT) c2(NP_001135936) c3(evolutionarily conserved signaling intermediate in Toll pathway, mitochondrial isoform 2 precursor) c4(53612) c5(53613, 53614, 53615, 53616, 53617, 53618) c6( )c7(K); #c1(ECSIT) c2(NP_001135937) c3(evolutionarily conserved signaling intermediate in Toll pathway, mitochondrial isoform 3 precursor) c4(53619) c5(53620, 53621, 53622, 53623, 53624, 53625) c6( )c7(K); #c1(ECSIT) c2(NP_001230133) c3(evolutionarily conserved signaling intermediate in Toll pathway, mitochondrial isoform 4 precursor) c4(85976) c5(85977, 85978, 85979, 85980, 85981) c6( )c7(K); #c1(ECSIT) c2(NP_057665) c3(evolutionarily conserved signaling intermediate in Toll pathway, mitochondrial isoform 1 precursor) c4(192218) c5(192219, 192220, 192221, 192222, 192223, 192224, 192225) c6( )c7(K); #c1(ECT2) c2(NP_001245244) c3(protein ECT2 isoform a) c4(92810) c5(92811, 92812, 92813, 92814, 92815, 92816, 92817, 92818, 92819, 92820) c6( )c7(abZ, iu, BI, T, d, aC, eM, py, eH, r, n, ey, K, g, eA, eF, fs, q); #c1(ECT2) c2(NP_001245245) c3(protein ECT2 isoform b) c4(92821, 92822, 92823) c5(92824, 92825, 92826, 92827, 92828, 92829, 92830, 92831, 92832, 92833, 92834, 92835, 92836, 92837, 92838, 92839, 92840, 92841, 92842, 92843, 92844, 92845, 92846, 92847, 92848, 92849, 92850, 92851) c6( )c7(abZ, iu, Bl, T, d, aC, eM, py, eH, r, n, ey, K, g, eA, eF, fs, q); #c1(ECT2) c2(XP_006713587) c3(protein ECT2 isoform X3) c4(287240) c5( )c6( )c7(abZ, iu, BI, T, d, aC, eM, py, eH, r, n, ey, K, g, eA, eF, fs, q); #c1(ECT2) c2(XP_011510817) c3(protein ECT2 isoform X1) c4( )c5(301280, 301281, 301282, 301283, 301284) c6( )c7(abZ, iu, BI, T, d, aC, eM, py, eH, r, n, ey, K, g, eA, eF, fs, q); #c1(ECT2) c2(XP_011510818) c3(protein ECT2 isoform X2) c4(301285, 301286) c5(301287, 301288, 301289, 301290, 301291, 301292) c6( )c7(abZ, iu, BI, T, d, aC, eM, py, eH, r, n, ey, K, g, eA, eF, fs, q); #c1(EDA) c2(NP_001005609) c3(ectodysplasin-A isoform 2) c4(21846) c5(21847, 21848, 21849, 21850, 21851, 21852, 21853, 21854, 21855, 21856, 21857, 21858, 21859, 21860, 21861, 21862, 21863, 21864, 21865, 21866, 21867, 21868, 21869, 21870, 21871, 21872, 21873, 21874, 21875, 21876, 21877, 21878, 21879, 21880, 21881, 21882, 21883, 21884, 21885, 21886, 21887, 21888, 21889, 21890, 21891, 21892, 21893, 21894, 21895, 21896, 21897, 21898, 21899, 21900, 21901, 21902, 21903, 21904, 21905, 21906, 21907, 21908, 21909, 21910, 21911, 21912, 21913, 21914, 21915, 21916, 21917, 21918, 21919, 21920, 21921, 21922, 21923, 21924, 21925, 21926, 21927, 21928, 21929, 21930, 21931, 21932, 21933, 21934, 21935, 21936, 21937, 21938, 21939, 21940, 21941) c6(21942, 21943, 21944) c7(T, sw, bjB, aml, bjz, bju, biA, b, bjA, bjv, tT, aT, lQ, Ha, bjy, aW, Y, eH, YF, eF, dr, bjw, bjx, Hs, dy, i, y); #c1(EDA) c2(NP_001005610) c3(ectodysplasin-A isoform 3) c4(21945) c5(21946, 21947, 21948, 21949, 21950, 21951, 21952, 21953) c6(21954, 21955) c7(T, sw, bjB, aml, bjz, bju, biA, b, bjA, bjv, tT, aT, lQ, Ha, bjy, aW, Y, eH, YF, eF, dr, bjw, bjx, Hs, dy, i, y); #c1(EDA) c2(NP_001005612) c3(ectodysplasin-A isoform 5) c4(21956) c5(21957, 21958, 21959, 21960, 21961, 21962, 21963, 21964) c6(21965, 21966) c7(T, sw, bjB, aml, bjz, bju, biA, b, bjA, bjv, tT, aT, lQ, Ha, bjy, aW, Y, eH, YF, eF, dr, bjw, bjx, Hs, dy, i, y); #c1(EDA) c2(NP_001005613) c3(ectodysplasin-A isoform 6) c4(21967) c5(21968, 21969, 21970, 21971, 21972, 21973, 21974, 21975, 21976, 21977, 21978, 21979, 21980, 21981) c6(21982, 21983, 21984, 21985) c7(T, sw, bjB, aml, bjz, bju, biA, b, bjA, bjv, tT, aT, lQ, Ha, bjy, aW, Y, eH, YF, eF, dr, bjw, bjx, Hs, dy, i, y); #c1(EDA) c2(NP_001390) c3(ectodysplasin-A isoform 1) c4(117524) c5(117525, 117526, 117527, 117528, 117529, 117530, 117531, 117532) c6(117533, 117534) c7(T, sw, bjB, aml, bjz, bju, biA, b, bjA, bjv, tT, aT, lQ, Ha, bjy, aW, Y, eH, YF, eF, dr, bjw, bjx, Hs, dy, i, y); #c1(EDA) c2(XP_011529187) c3(ectodysplasin-A isoform X2) c4(324591) c5( )c6( )c7(T, sw, bjB, aml, bjz, bju, biA, b, bjA, bjv, tT, aT, lQ, Ha, bjy, aW, Y, eH, YF, eF, dr, bjw, bjx, Hs, dy, i, y); #c1(EDA2R) c2(NP_001186616) c3(tumor necrosis factor receptor superfamily member 27 isoform 1) c4(77973) c5(77974, 77975, 77976, 77977, 77978, 77979, 77980, 77981, 77982, 77983, 77984, 77985, 77986, 77987) c6( )c7(Ln, aW, r, aT, aeM, q); #c1(EDA2R) c2(XP_011529303) c3(tumor necrosis factor receptor superfamily member 27 isoform X1) c4(324727) c5(324728, 324729, 324730, 324731, 324732, 324733, 324734, 324735, 324736, 324737) c6( )c7(Ln, aW, r, aT, aeM, q); #c1(EDA2R) c2(XP_011529307) c3(tumor necrosis factor receptor superfamily member 27 isoform X6) c4(324738) c5(324739, 324740, 324741, 324742, 324743, 324744) c6( )c7(Ln, aW, r, aT, aeM, q); #c1(EDAR) c2(NP_07/731) c3(tumor necrosis factor receptor superfamily member EDAR precursor) c4(211845) c5(211846, 211847, 211848, 211849, 211850) c6( )c7(bjy, bjA, bjv, aW, bjB, bjC, aT, aml, af); #c1(EDAR) c2(XP_006712267) c3(tumor necrosis factor receptor superfamily member EDAR isoform X2) c4(286588) c5(286589, 286590, 286591, 286592, 286593, 286594, 286595, 286596) c6( )c7(bjy, bjA, bjv, aW, bjB, bjC, aT, aml, af); #c1(EDAR) c2(XP_011508804) c3(tumor necrosis factor receptor superfamily member EDAR isoform X1) c4( )c5(299401, 299402) c6( ) c7(bjy, bjA, bjv, aW, bjB, bjC, aT, aml, af); #c1(EDAR) c2(XP_011508805) c3(tumor necrosis factor receptor superfamily member EDAR isoform X3) c4( )c5(299403) c6( )c7(bjy, bjA, bjv, aW, bjB, bjC, aT, aml, af); #c1(EDARADD) c2(NP_542776) c3(ectodysplasin-A receptor-associated adapter protein isoform B) c4(233037) c5(233038, 233039, 233040, 233041, 233042, 233043, 233044, 233045, 233046, 233047, 233048) c6( )c7(bjD, bjA, bjv, bjC, bjE, aml); #c1(EDARADD) c2(NP_665860) c3(ectodysplasin-A receptor-associated adapter protein isoform A) c4(241848, 241849) c5(241850, 241851, 241852, 241853, 241854, 241855) c6( )c7(bjD, bjA, bjv, bjC, bjE, aml); #c1(EDC4) c2(NP_055144) c3(enhancer of mRNA-decapping protein 4) c4(179400, 179401) c5(179402, 179403, 179404, 179405, 179406, 179407, 179408, 179409, 179410, 179411, 179412, 179413) c6( )c7(om, j); #c1(EDEM1) c2(NP_055489) c3(ER degradation-enhancing alpha-mannosidase-like protein 1) c4(181478) c5(181479, 181480, 181481, 181482, 181483, 181484, 181485) c6( )c7(Ei, oe, Uq, K, b0); #c1(EDEM1) c2(XP_011532574) c3(ER degradation-enhancing alpha-mannosidase-like protein 1 isoform X3) c4(328684, 328685) c5(328686, 328687, 328688, 328689, 328690, 328691, 328692, 328693, 328694, 328695, 328696) c6( )c7(Ei, oe, Uq, K, b0); #c1(EDIL3) c2(NP_001265571) c3(EGF-like repeat and discoidin I-like domain-containing protein 3 isoform 2 precursor) c4(98864) c5(98865, 98866, 98867, 98868, 98869, 98870, 98871, 98872) c6(98873, 98874) c7(T, os, jv, iW, oe, ha, cz, bB, oZ, v, j, q, x, Mg, lg, fm, bhF, M, aT, dL, K, Dz, eL, mR, aW, Y, iV, uC, ek, W, bE, wk, Fk, k, bt, B); #c1(EDIL3) c2(NP_005702) c3(EGF-like repeat and discoidin I-like domain-containing protein 3 isoform 1 precursor) c4(158654) c5(158655, 158656, 158657, 158658, 158659, 158660, 158661, 158662) c6(158663, 158664) c7(T, os, jv, iW, oe, ha, cz, bB, oZ, v, j, q, x, Mg, lg, fm, bhF, M, aT, dL, K, Dz, eL, mR, aW, Y, iV, uC, ek, W, bE, wk, Fk, k, bt, B); #c1(EDN1) c2(NP_001946) c3(endothelin-1 isoform 1 preproprotein) c4(122438) c5(122439, 122440, 122441, 122442, 122443, 122444) c6( )c7(oO, gh, G, T, bM, lD, ea, if, e, al, eb, bB, fo, ur, ci, qj, lR, aY, Jf, YD, ip, Na, aif, xy, Bc, dV, iv, Ds, K, bjF, aDU, aCN, bc, hm, aa, eL, ly, bjJ, dH, aW, Az, hl, aB, bjH, as, eF, cm, uC, iA, nB, AM, vA, mx, vy, cj, bjL, wB, wx, bgd, Hb, m, mk, hh, fy, aCS, cZ, bfb, dD, aw, bfx, BH, aj, fr, uE, bjl, qN, cd, TB, cc, hw, ec, acA, kl, Lv, lG, h, dl, vd, wb, ey, dd, wo, vx, Ha, WC, ES, sp, fQ, fe, CA, mo, dw, ban, op, lx, ar, kT, Sc, cA, wk, hL, tl, E, te, bjk, uz, va, at, bD, sZ, B, fl, vQ, bL, Dw, eR, aYP, wL, fG, oe, uv, cz, cR, tc, fF, uZ, j, v, C, sl, bX, Cb, q, J, eA, aRo, UE, oT, hb, afQ, aT, cy, Nc, bq, an, DX, Fg, ae, af, dl, qk, eu, ak, Sh, bf, s, Y, bg, r, en, cl, t, pb, vk, nM, kP, ua, c, ac, wl, hc, DW, hr, fP, dy, ao, sY, kh, wi, qb, uG, jh, bjG, aZH, mw, dG, uu, eq, Fb, ad, BY, av, hd, py, sN, oz, hD, vw, vc, Aa, aNp, jo, nJ, sj, d, lR, qZ, cH, cW, aU, ff, eH, cr, eZ, dq, si, Fq, bz, qa, LB, D, Hw, gk, zr, bE, z, X, gZ, Hs, gB, aS, bC, oQ, ei, lC, vt, gq, y, pC); #c1(EDN3) c2(NP_001289385) c3(endothelin-3 isoform 5 preproprotein) c4(114/71) c5(114/72, 114/73, 114/74, 114175, 114/76, 114/77) c6( )c7(d, bE, aW, Y, aT, ahF, amj, cz, bB, bjM, bjN, aPF, ahl, aTa, bjO, ahE, ao); #c1(EDN3) c2(NP_996915) c3(endothelin-3 isoform 2 preproprotein) c4(264871) c5(264872, 264873, 264874, 264875, 264876, 264877) c6( )c7(d, bE, aW, Y, aT, ahF, amj, cz, bB, bjM, bjN, aPF, ahl, aTa, bjO, ahE, ao); #c1(EDN3) c2(NP_996916) c3 (endothelin-3 isoform 3 preproprotein) c4(264878) c5(264879, 264880, 264881, 264882, 264883, 264884) c6( )c7(d, bE, aW, Y, aT, ahF, amj, cz, bB, bjM, bjN, aPF, ahl, aTa, bjO, ahE, ao); #c1(EDN3) c2(NP_996917) c3(endothelin-3 isoform 1 preproprotein) c4(264885) c5(264886, 264887, 264888, 264889, 264890, 264891, 264892, 264893, 264894, 264895) c6( )c7(d, bE, aW, Y, aT, ahF, amj, cz, bB, bjM, bjN, aPF, ahl, aTa, bjO, ahE, ao); #c1(EDNRA) c2(NP_001159527) c3(endothelin-1 receptor isoform b precursor) c4(64228) c5(64229, 64230, 64231, 64232, 64233, 64234, 64235, 64236, 64237, 64238, 64239, 64240, 64241, 64242, 64243) c6( )c7(ID, bM, T, ea, e, al, fd, Ja, ur, ci, aY, xY, ye, bv, xy, Bc, K, Nj, tw, ly, aW, Az, Qv, bjH, g, as, eF, cm, iP, qJ, y, eM, iA, wE, vA, mx, wB, cj, bgd, Hb, m, bim, aCS, cZ, dD, aw, akA, fr, qN, cd, yn, TB, jz, cc, Mv, h, ey, wC, yi, A, sp, yc, yq, CQ, mo, dw, vo, op, lx, jq, ar, kT, ok, yf, bP, hk, wk, tl, at, te, bD, sZ, B, it, vQ, bL, iH, dA, eR, yk, hU, jx, aA, cR, tc, cz, v, j, aGK, yp, eA, xW, aT, xV, bK, yb, bq, yj, ae, af, xU, s, Y, LU, t, nC, iK, pb, nM, ac, yl, dy, bt, ao, HQ, ha, Me, uG, uu, ah, eq, bjQ, ad, BY, av, hd, agd, db, hD, yh, bjP, nJ, xX, d, qZ, eH, aF, si, bz, qa, dR, D, gk, xZ, aS, od, lC, us); #c1(EDNRA) c2(NP_001948) c3(endothelin-l receptor isoform a precursor) c4(122445, 122446, 122447) c5(122448, 122449, 122450, 122451, 122452, 122453, 122454, 122455, 122456, 122457, 122458, 122459, 122460) c6( )c7(ID, bM, T, ea, e, al, fd, Ja, ur, ci, aY, xY, ye, bv, xy, Bc, K, Nj, tw, ly, aW, Az, Av, bjH, g, as, eF, cm, iP, qJ, y, eM, iA, wE, vA, mx, wB, cj, bgd, Hb, m, bim, aCS, cZ, dD, aw, akA, fr, qN, cd, yn, TB, jz, cc, Mv, h, ey, wC, yi, A, sp, yc, yq, CA, mo, dw, vo, op, lx, jq, ar, kT, ok, yf, bP, hk, wk, tl, at, te, bD, sZ, B, it, vQ, bL, iH, dA, eR, yk, hU, jx, aA, cR, tc, cz, v, j, aGK, yp, eA, xW, aT, xV, bK, yb, bq, yj, ae, af, xU, s, Y, LU, t, nC, ik, pb, nM, ac, yl, dy, bt, ao, HA, ha, Me, uG, uu, ah, eq, bjQ, ad, BY, av, hd, agd, db, hD, yh, bjP, nJ, xX, d, qZ, eH, af, si, bz, qa, dR, D, gk, xZ, aS, ol, lC, us); #c1(EDNRB) c2(NP_000106) c3 (endothelin B receptor isoform 1 precursor) c4(1815) c5( )c6( )c7(ID, bM, T, abY, aC, e, al, ur, ci, bjN, lR, ip, bjV, Bc, iv, eL, cp, aa, ly, aW, wf, bjH, g, as, eF, cm, iP, eM, vA, Ji, mx, bjU, bim, aCS, cZ, dD, aw, fr, cd, aJe, TB, aJf, ft, jz, cc, lG, dL, cY, wC, bjR, yq, mo, dw, QK, op, Ix, hk, wk, hL, at, te, va, nz, Pr, B, it, bL, jx, cz, tc, j, aGK, eA, aT, aPF, ahE, ae, af, s, Y, als, LU, en, ahl, cl, f, ik, pb, ck, ac, dy, ao, rd, uG, Fb, oH, uu, eq, Fc, ad, BY, av, py, bjS, bjT, bjP, nJ, d, ff, eH, gu, aTa, bz, D, gk, Hs, lC, y); #c1(EDNRB) c2(NP_001188326) c3(endothelin B receptor isoform 3) c4(79056) c5(79057, 79058, 79059, 79060, 79061, 79062, 79063, 79064, 79065, 79066, 79067, 79068) c6( ) c7(ID, bM, T, abY, aC, e, al, ur, ci, bjN, lR, ip, bjV, Bc, iv, eL, cp, aa, ly, aW, wF, bjH, g, as, eF, cm, iP, eM, vA, Ji, mx, bju, bim, aCS, cZ, dD, aw, fr, cd, aJe, TB, aJf, ft, jz, cc, lG, dL, cY, wC, bjR, yq, mo, dw, QK, op, lx, hk, wk, hl, at, te, va, nz, Pr, B, it, bL, jx, cz, tc, j, aGK, eA, aT, aPF, ahE, ae, af, s, Y, aZs, LU, en, ahl, cl, f, ik, pb, ck, ac, dy, ao, rd, uG, Fb, oH, uu, eq, Fc, ad, BY, av, py, bjS, bjT, bjP, nJ, d, ff, eH, gu, aTa, bz, D, gk, Hs, lC, y); #c1(EDNRB) c2(NP_003982) c3(endothelin B receptor isoform 2 precursor) c4(142637) c5(142638, 142639, 142640, 142641, 142642, 142643) c6( )c7(ID, bM, T, abY, aC, e, al, ur, ci, bjN, lR, ip, bjV, Bc, iv, eL, cp, aa, ly, aW, wf, bjH, g, as, eF, cm, iP, eM, vA, Ji, mx, bjU, bim, aCS, cZ, dD, aw, fr, cd, aJe, TB, aJf, ft, jz, cc, lG, dL, cY, wC, bjR, yq, mo, dw, QK, op, lx, hk, wk, hl, at, te, va, nz, Pr, B, it, bL, jx, cz, tc, j, aGK, eA, aT, aPF, ahE, ae, af, s, Y, aZs, LU, en, ahl, cl, f, ik, pb, ck, ac, dy, ao, rd, uG, Fb, oH, uu, eq, Fc, ad, BY, av, py, bjS, bjT, bjP, nJ, d, ff, eH, gu, aTa, bz, D, gk, Hs, lC, y); #c1(EEA1) c2(NP_003557) c3(early endosome antigen 1) c4(139181, 139182) c5(139183, 139184, 139185, 139186, 139187, 139188, 139189, 139190, 139191, 139192, 139193, 139194) c6( )c7(h, aj, J); #c1(EED) c2(NP_003788) c3(polycomb protein EED isoform a) c4(140805, 140806) c5(140807, 140808, 140809, 140810, 140811, 140812, 140813, 140814, 140815, 140816, 140817, 140818, 140819, 140820) c6( )c7(T, aW, ael, aT, aq, vs, hP, iV, dx); #c1(EED) c2(XP_005274430) c3(polycomb protein EED isoform X4) c4( )c5(285077, 285078, 285079, 285080, 285081) c6( )c7(T, aW, aeO, aT, aq, vs, hP, iV, dx); #c1(EEF1A1) c2(XP_011533816) c3(elongation factor 1-alpha 1 isoform X1) c4(329939, 329940, 329941, 329942, 329943) c5(329944, 329945, 329946, 329947, 329948, 329949, 329950, 329951, 329952, 329953, 329954, 329955, 329956, 329957, 329958, 329959, 329960, 329961, 329962, 329963, 329964, 329965, 329966, 329967, 329968, 329969, 329970, 329971, 329972, 329973, 329974, 329975, 329976, 329977, 329978, 329979, 329980, 329981) c6( )c7(T, wu, aA, bB, bjW, ci, zP, q, ad, ft, aE, gH, aT, K, wm, cb, zy, ae, yi, zl, vT, aW, Y, r, fe, t, bz, bE, m, B); #c1(EEF1A2) c2(NP_001949) c3(elongation factor 1-alpha 2) c4(122461, 122462) c5(122463, 122464, 122465, 122466, 122467, 122468, 122469, 122470, 122471, 122472, 122473, 122474, 122475, 122476, 122477, 122478) c6( )c7(bM, T, ea, e, fd, bB, fo, ci, dY, M, K, dQ, hm, aW, g, eF, cj, m, dD, tp, aw, NT, n, aj, no, pe, ec, eV, kp, kT, W, Fi, at, B, L, bL, jx, DV, ex, aA, cR, v, j, V, q, J, aT, cy, jj, ae, af, bA, wr, bf, s, Y, r, lo, t, ck, rR, tW, ac, ao, hS, bjX, qb, Dd, ha, aHv, bjW, eq, ad, tX, F, sO, oj, uX, d, cH, cW, aaB, eH, gC, cr, eZ, Bu, bz, bE, Om, aS, ei, Zw, iu, y); #c1(EEF1B2) c2(NP_001032752) c3(elongation factor 1-beta) c4(31454, 31455) c5(31456, 31457, 31458, 31459, 31460, 31461, 31462, 31463, 31464, 31465, 31466, 31467, 31468, 31469, 31470, 31471, 31472, 31473, 31474, 31475) c6( )c7(T, nA, aT); #c1(EEF1D) c2(NP_001123525) c3(elongation factor 1-delta isoform 1) c4(47332, 47333, 47334, 47335, 47336, 47337, 47338, 47339, 47340, 47341, 47342) c5(47343, 47344, 47345, 47346, 47347, 47348, 47349, 47350, 47351, 47352, 47353, 47354, 47355, 47356, 47357, 47358, 47359, 47360, 47361, 47362, 47363, 47364, 47365, 47366, 47367, 47368, 47369, 47370, 47371, 47372, 47373, 47374, 47375, 47376, 47377, 47378, 47379, 47380, 47381, 47382, 47383, 47384, 47385, 47386, 47387, 47388, 47389, 47390, 47391, 47392, 47393, 47394, 47395, 47396, 47397, 47398, 47399, 47400, 47401, 47402, 47403, 47404) c6( )c7(jf, T, aT, kf, ci, hu); #c1(EEF1D) c2(NP_001123528) c3(elongation factor 1-delta isoform 4) c4(47405) c5(47406, 47407, 47408, 47409, 47410, 47411, 47412, 47413, 47414, 47415, 47416, 47417, 47418, 47419, 47420, 47421, 47422, 47423, 47424, 47425, 47426, 47427, 47428) c6( )c7(jf, T, aT, kf, ci, hu); #c1(EEF1D) c2(NP_001182132) c3(elongation factor 1-delta isoform 5) c4(74609, 74610) c5(74611, 74612, 74613, 74614, 74615, 74616, 74617, 74618, 74619, 74620, 74621, 74622) c6( )c7(jf, T, aT, kf, ci, hu); #c1(EEF1D) c2(NP_001951) c3(elongation factor 1-delta isoform 2) c4(122479, 122480, 122481, 122482, 122483, 122484, 122485) c5(122486, 122487, 122488, 122489, 122490, 122491, 122492, 122493, 122494, 122495, 122496, 122497, 122498, 122499, 122500, 122501, 122502, 122503, 122504, 122505, 122506, 122507, 122508, 122509, 122510, 122511, 122512, 122513, 122514, 122515, 122516, 122517, 122518, 122519, 122520, 122521, 122522, 122523, 122524, 122525, 122526, 122527, 122528, 122529, 122530, 122531, 122532, 122533, 122534, 122535, 122536, 122537) c6( )c7(jf, T, aT, kf, ci, hu); #c1(EEF1D) c2(XP_006725127) c3(elongation factor 1-delta isoform X1) c4(296613) c5(296614, 296615, 296616, 296617, 296618, 296619, 296620, 296621, 296622, 296623, 296624, 296625, 296626, 296627, 296628, 296629, 296630) c6( )c7(jf, T, aT, kf, ci, hu); #c1(EEF1D) c2(XP_006725128) c3(elongation factor 1-delta isoform X2) c4(296631) c5(296632, 296633) c6( )c7(jf, T, aT, kf, ci, hu); #c1(EEF1D) c2(XP_006725129) c3(elongation factor 1-delta isoform X3) c4(296634, 296635, 296636) c5(296637, 296638, 296639, 296640, 296641, 296642, 296643, 296644, 296645, 296646, 296647, 296648, 296649, 296650, 296651, 296652) c6( )c7(jf, T, aT, kf, ci, hu); #c1(EEF1D) c2(XP_006725135) c3(elongation factor 1-delta isoform X4) c4(296653, 296654, 296655, 296656, 296657, 296658) c5(296659, 296660, 296661, 296662, 296663, 296664, 296665, 296666, 296667, 296668, 296669, 296670, 296671, 296672, 296673, 296674, 296675, 296676, 296677, 296678, 296679, 296680, 296681, 296682, 296683, 296684, 296685, 296686, 296687, 296688, 296689, 296690, 296691, 296692, 296693, 296694) c6( )c7(jf, T, aT, kf, ci, hu); #c1(EEF1E1) c2(NP_001129122) c3(eukaryotic translation elongation factor 1 epsilon-1 isoform 2) c4(50838) c5(50839, 50840, 50841, 50842, 50843, 50844) c6( )c7(ck, eM, Y, iS, fd, bw, dL, v, K, hF, vt, eL, B, ip); #c1 (EEF1E1) c2(NP_004271) c3(eukaryotic translation elongation factor 1 epsilon-1 isoform 1) c4(144750) c5(144751, 144752, 144753, 144754, 144755, 144756) c6( )c7(ck, eM, Y, iS, fd, bw, dL, v, K, hF, vt, eL, B, ip); #c1(EEF1G) c2(NP_001395) c3(elongation factor 1-gamma) c4(117535, 117536, 117537, 117538) c5(117539, 117540, 117541, 117542, 117543, 117544, 117545, 117546, 117547, 117548, 117549, 117550, 117551, 117552, 117553, 117554, 117555, 117556, 117557, 117558, 117559, 117560, 117561, 117562) c6( )c7(bF, aC, aE, bE, r, g, hu, q); #c1(EEF2) c2(NP_001952) c3(elongation factor 2) c4(122538, 122539) c5(122540, 122541, 122542, 122543, 122544, 122545, 122546, 122547, 122548, 122549, 122550, 122551) c6( )c7(av, T, ayl, yt, U, aT, r, wR, fr, g, cM, af, ad); #c1 (EEFSEC) c2(NP_068756) c3(selenocysteine-specific elongation factor) c4(210195) c5(210196, 210197, 210198, 210199, 210200, 210201) c6( )c7(cd, ad); #c1(EFCAB1) c2(XP_005251361) c3(EF-hand calcium-binding domain-containing protein 1 isoform X2) c4(270910) c5(270911, 270912, 270913, 270914, 270915, 270916, 270917, 270918, 270919, 270920) c6( )c7(cz); #c1(EFCAB1) c2(XP_011515891) c3(EF-hand calcium-binding domain-containing protein 1 isoform X1) c4(308984, 308985) c5(308986, 308987, 308988, 308989, 308990, 308991, 308992, 308993, 308994, 308995) c6( )c7(cz); #c1(EFCAB1) c2(XP_011515893) c3(EF-hand calcium-binding domain-containing protein 1 isoform X3) c4( )c5(308996, 308997, 308998, 308999, 309000, 309001) c6( )c7(cz); #c1(EFCAB11) c2(NP_001271195) c3(EF-hand calcium-binding domain-containing protein 11 isoform 2) c4( )c5(103550) c6( )c7(hH, B); #c1(EFCAB11) c2(NP_001271197) c3(EF-hand calcium-binding domain-containing protein 11 isoform 4) c4(103551, 103552) c5(103553, 103554, 103555, 103556, 103557, 103558, 103559, 103560, 103561, 103562, 103563) c6( )c7(hH, B); #c1(EFCAB11) c2(NP_001271198) c3(EF-hand calcium-binding domain-containing protein 11 isoform 5) c4(103564) c5(103565, 103566, 103567, 103568, 103569, 103570) c6( )c7(hH, B); #c1(EFCAB11) c2(NP_660274) c3(EF-hand calcium-binding domain-containing protein 11 isoform 1) c4(240585) c5(240586, 240587, 240588, 240589, 240590, 240591) c6( )c7(hH, B); #c1(EFEMP1) c2(NP_001034438) c3(EGF-containing fibulin-like extracellular matrix protein 1 precursor) c4(31664, 31665, 31666) c5(31667, 31668, 31669, 31670, 31671, 31672, 31673, 31674, 31675, 31676, 31677, 31678, 31679, 31680, 31681, 31682, 31683, 31684, 31685, 31686, 31687, 31688, 31689, 31690, 31691, 31692, 31693, 31694, 31695, 31696, 31697, 31698, 31699, 31700, 31701, 31702, 31703, 31704, 31705, 31706, 31707, 31708, 31709, 31710, 31711, 31712, 31713, 31714, 31715, 31716) c6( )c7(T, dD, bjZ, aC, if, nx, n, aj, eq, ci, qN, zP, q, J, cd, eA, eJ, av, b, aE, aqT, i, Sr, aT, agD, bka, db, Vj, qw, yo, zl, d, lR, s, wr, aW, r, g, cl, t, bz, dr, W, nb, c, eM, bjY, aqV, nw, m, GF, xb, B); #c1(EFEMP1) c2(XP_005264262) c3(EGF-containing fibulin-like extracellular matrix protein 1 isoform X1) c4(278744, 278745, 278746, 278747, 278748, 278749, 278750) c5(278751, 278752, 278753, 278754, 278755, 278756, 278757, 278758, 278759, 278760, 278761, 278762, 278763, 278764, 278765, 278766, 278767, 278768) c6( )c7(T, dD, bjZ, aC, if, nx, n, aj, eq, ci, qN, zP, q, J, cd, eA, eJ, av, b, aE, aqT, i, Sr, aT, agD, bka, db, Vj, qw, yo, zQ, d, lR, s, wr, aW, r, g, cl, t, bz, dr, W, nb, c, eM, bjY, aqV, nw, m, GF, xb, B); #c1(EFEMP2) c2(NP_058634) c3(EGF-containing fibulin-like extracellular matrix protein 2 precursor) c4(192864, 192865, 192866) c5(192867, 192868, 192869, 192870, 192871, 192872, 192873, 192874, 192875, 192876, 192877, 192878, 192879, 192880, 192881, 192882, 192883, 192884, 192885, 192886, 192887, 192888, 192889, 192890, 192891, 192892, 192893, 192894, 192895, 192896) c6( )c7(bkb, T, y, lR, lP, Uj, aW, bkc, xs, bjY, aT, Aa, aS, ci, pb, QK, X, dr, ad); #c1(EFHB) c2(NP_653316) c3(EF-hand domain-containing family member B) c4(239565) c5(239566, 239567, 239568, 239569, 239570, 239571, 239572, 239573) c6(239574, 239575) c7(A); #c1(EFHB) c2(XP_011531685) c3(EF-hand domain-containing family member B isoform X3) c4( )c5(327793) c6( )c7(A); #c1(EFHC1) c2(NP_001165891) c3(EF-hand domain-containing protein 1 isoform 2) c4(67944, 67945) c5(67946, 67947, 67948, 67949, 67950, 67951, 67952, 67953, 67954, 67955, 67956, 67957, 67958) c6( )c7(pa, Cq, ha, HN, iD, avl, uu, hg, bkd, sj); #c1(EFHC1) c2(NP_060570) c3(EF-hand domain-containing protein 1 isoform 1) c4(196623) c5(196624, 196625, 196626, 196627, 196628, 196629) c6( )c7(pa, Cq, ha, HN, iD, avl, uu, hg, bkd, sj); #c1(EFHC2) c2(NP_079460) c3(EF-hand domain-containing family member C2) c4(218179, 218180, 218181) c5(218182, 218183, 218184, 218185, 218186, 218187, 218188, 218189, 218190, 218191, 218192, 218193, 218194, 218195, 218196, 218197, 218198, 218199, 218200) c6( )c7(vJ, iD, agZ); #c1(EFHC2) c2(XP_006724625) c3(EF-hand domain-containing family member C2 isoform X2) c4(296134, 296135) c5(296136, 296137, 296138, 296139, 296140, 296141, 296142, 296143, 296144, 296145, 296146) c6( )c7(vJ, iD, agZ); #c1(EFHD1) c2(NP_001230181) c3(EF-hand domain-containing protein DI isoform 2) c4(86067) c5(86068, 86069, 86070, 86071, 86072, 86073, 86074, 86075) c6( )c7(qK); #c1(EFHD1) c2(NP_079478) c3(EF-hand domain-containing protein DI isoform 1) c4(218251, 218252, 218253) c5(218254, 218255, 218256, 218257, 218258, 218259, 218260, 218261, 218262, 218263, 218264, 218265) c6( )c7(qK); #c1(EFNA1) c2(NP_004419) c3 (ephrin-A1 isoform a precursor) c4(146226) c5(146227, 146228, 146229, 146230, 146231, 146232) c6( )c7(bM, T, id, aA, n, ci, q, eA, iu, aT, dL, eL, hm, cY, Qe, s, nO, aW, Y, ff, r, aq, aF, en, uC, bz, eM, bE, m, B); #c1(EFNA1) c2(NP_872626) c3(ephrin-A1 isoform b precursor) c4(256551) c5(256552, 256553, 256554, 256555, 256556, 256557) c6( )c7(bM, T, id, aA, n, ci, q, eA, iu, aT, dl, eL, hm, cY, Qe, s, nO, aW, Y, ff, r, aq, aF, en, uC, bz, eM, bE, m, B); #c1(EFNA2) c2(NP_001396) c3(ephrin-A2 precursor) c4(117563) c5(117564, 117565, 117566, 117567, 117568, 117569) c6( )c7(bP, if, eB, gX, eZ, h, qN, cl, bt); #c1(EFNA3) c2(NP_004943) c3(ephrin-A3 precursor) c4(151339, 151340, 151341) c5(151342, 151343, 151344, 151345, 151346, 151347, 151348, 151349, 151350, 151351, 151352, 151353, 151354, 151355, 151356, 151357, 151358, 151359, 151360, 151361, 151362, 151363) c6( )c7(bz, af); #c1(EFNA4) c2(NP_005218) c3(ephrin-A4 isoform a precursor) c4(154116) c5(154117, 154118, 154119, 154120, 154121, 154122) c6( )c7(atL, agj, sH, cj); #c1(EFNA4) c2(NP_872631) c3(ephrin-A4 isoform b precursor) c4(256577) c5(256578, 256579, 256580, 256581, 256582, 256583) c6( )c7(atL, agj, sH, cj); #c1(EFNA4) c2(NP_872632) c3(ephrin-A4 isoform c precursor) c4(256584) c5(256585, 256586, 256587, 256588, 256589, 256590) c6( )c7(atL, agj, sH, cj); #c1(EFNA5) c2(NP_001953) c3(ephrin-A5 precursor) c4(122552) c5(122553, 122554, 122555, 122556, 122557, 122558) c6( )c7(T, kh, ac, dY, aW, Y, rl, aT, rM, lG, eZ, fd, k, ux, an, Gp, he); #c1 (EFNA5) c2(XP_011541553) c3(ephrin-A5 isoform X3) c4( )c5(340103) c6( )c7(T, kh, ac, dY, aW, Y, rl, aT, rM, lG, eZ, fd, k, ux, an, Gp, he); #c1(EFNB1) c2(NP_004420) c3(ephrin-B1 precursor) c4(146233) c5(146234, 146235, 146236, 146237, 146238, 146239) c6( )c7(T, dD, ea, aA, v, eq, nA, bjz, q, bke, bjA, bjv, TR, jf, d, Az, r, aF, g, as, eF, zV, ck, wk, y, B, sH); #c1(EFNB2) c2(NP_004084) c3(ephrin-B2 precursor) c4(143421) c5(143422, 143423, 143424, 143425, 143426, 143427) c6( )c7(bL, T, eR, fR, e, aA, bB, ci, aY, q, eA, hd, aT, h, lG, cy, hu, d, lR, B, nO, aW, ff, aeq, r, aq, aF, yq, en, as, cm, ck, nB, bE, dy, it, X); #c1(EFNB3) c2(NP_001397) c3(ephrin-B3 precursor) c4(117570) c5(117571, 117572, 117573, 117574, 117575, 117576) c6( )c7(d, eM, adR, dl, ci, cm, eA); #c1(EFR3A) c2(NP_055952) c3(protein EFR3 homolog A) c4(184645, 184646, 184647) c5(184648, 184649, 184650, 184651, 184652, 184653, 184654, 184655, 184656, 184657, 184658, 184659, 184660, 184661, 184662, 184663, 184664, 184665, 184666, 184667, 184668, 184669, 184670, 184671) c6( )c7(ey); #c1(EFR3A) c2(XP_005250906) c3(protein EFR3 homolog A isoform X2) c4(270618, 270619) c5(270620, 270621, 270622, 270623, 270624) c6( )c7(ey); #c1(EFR3A) c2(XP_011515247) c3(protein EFR3 homolog A isoform X1) c4( ) c5(308063) c6( )c7(ey); #c1(EFR3B) c2(NP_055786) c3(protein EFR3 homolog B) c4(183486) c5(183487, 183488, 183489, 183490, 183491, 183492, 183493, 183494, 183495, 183496, 183497, 183498) c6( )c7(A); #c1(EFR3B) c2(XP_011531005) c3(protein EFR3 homolog B isoform X3) c4(326965) c5( )c6( )c7(A); #c1(EFS) c2(NP_001264103) c3(embryonal Fyn-associated substrate isoform 3) c4(97529) c5(97530, 97531, 97532, 97533, 97534, 97535, 97536) c6( )c7(ck, Tg, T, x, fX, bkf, Y, h, k, CQ, cm, bt, fh, er, iP); #c1(EFS) c2(NP_005855) c3(embryonal Fyn-associated substrate isoform 1) c4(159939) c5(159940, 159941, 159942, 159943, 159944, 159945) c6( )c7(ck, Tg, T, x, fx, bkf, Y, h, k, CQ, cm, bt, fh, er, iP); #c1(EFS) c2(NP_115835) c3(embryonal Fyn-associated substrate isoform 2) c4(224252) c5(224253, 224254, 224255, 224256, 224257, 224258) c6( )c7(ck, Tg, T, x, fx, bkf, Y, h, k, CQ, cm, bt, fh, er, iP); #c1 (EFTUD1) c2(NP_001035700) c3(elongation factor Tu GTP-binding domain-containing protein 1 isoform 2) c4(32948, 32949, 32950) c5(32951, 32952, 32953, 32954, 32955, 32956, 32957, 32958) c6( )c7(aw, dY); #c1(EFTUD1) c2(NP_078856) c3(elongation factor Tu GTP-binding domain-containing protein 1 isoform 1) c4(215787) c5(215788, 215789, 215790, 215791, 215792, 215793, 215794, 215795, 215796, 215797, 215798, 215799, 215800, 215801, 215802, 215803) c6( )c7(aw, dY); #c1(EFTUD2) c2(NP_001136077) c3(116 kDa US small nuclear ribonucleoprotein component isoform b) c4(54272) c5( )c6( )c7(bkg, aMU, Xg, bkh, QK, nC); #c1(EFTUD2) c2(NP_001245282) c3(116 kDa U5 small nuclear ribonucleoprotein component isoform a) c4(92970, 92971) c5(92972, 92973, 92974, 92975, 92976, 92977, 92978, 92979, 92980, 92981, 92982, 92983, 92984, 92985, 92986, 92987, 92988, 92989) c6( )c7(bkg, aMU, Xg, bkh, QK, nC); #c1(EGF) c2(NP_001171601) c3(pro-epidermal growth factor isoform 2 preproprotein) c4(70717) c5(70718, 70719, 70720, 70721, 70722, 70723, 70724) c6( )c7(ID, gh, G, T, bM, aC, pf, hV, ea, if, e, al, bO, eb, ajp, bB, axk, ahX, lR, ci, fd, aY, ip, x, Vp, bv, Na, aE, rJ, pX, aeO, bki, dV, auB, iv, ja, K, Nj, hu, bkk, eL, aa, ly, xQ, yo, bkr, ZH, aeR, JI, aW, aoo, Ls, hl, LZ, Qm, YG, PQ, g, as, eE, eF, uC, cm, Qt, zV, iP, bw, aAW, qJ, eM, nB, AM, nF, iA, vA, wB, cj, NI, dW, m, fy, cE, iu, Vj, dD, xu, vl, aEj, bkq, sw, zt, ht, NT, aBx, n, kv, fr, qN, auA, aje, asS, eW, El, ft, no, jz, Yc, bkm, ec, i, Nn, gd, lG, By, dL, ey, hB, Ha, Dg, asp, RT, CN, MD, As, hQ, Vt, ack, aq, kp, fe, auC, op, lx, KT, bkn, W, GU, dB, hk, eD, ajn, hL, gw, uz, hR, at, it, B, bL, eR, Dh, fG, WT, jx, aA, cz, fF, hy, v, yO, asA, bX, bkj, q, eA, qH, XX, Mg, mj, anA, lg, bjA, nv, Qg, qp, BJ, aT, kf, cy, anz, an, rz, ae, ne, bkl, dl, amx, s, ND, nO, oV, Y, ev, LO, il, r, lo, tu, en, bu, cl, t, apx, bkp, pb, ik, bN, wA, tR, nM, kP, c, Dy, ac, wO, ll, nG, gX, WU, adR, CZ, dy, bko, anD, im, bt, ao, hS, On, hl, aD, jN, uO, jh, Hn, bjW, Fb, eq, jE, aml, ad, wD, hX, dP, av, iC, hd, F, ib, py, xl, yt, db, NS, tT, Sr, bj, Dj, hP, ji, ako, nJ, Yi, d, vj, ff, eH, cr, dN, tv, and, aF, hp, Fq, Pd, iR, bz, LB, es, D, gk, pu, arY, TA, kg, bE, Ew, AR, wz, Hs, adg, lC, X, y); #c1(EGF) c2(NP_001171602) c3(pro-epidermal growth factor isoform 3 preproprotein) c4(70725) c5(70726, 70727, 70728, 70729, 70730, 70731, 70732) c6( )c7(ID, gh, G, T, bM, aC, pf, hV, ea, if, e, al, bO, eb, ajp, bB, axk, ahX, lR, ci, fd, aY, ip, x, Vp, bv, Na, aE, rJ, pX, aeO, bki, dV, auB, iv, ja, K, Nj, hu, bkk, eL, aa, ly, xQ, yo, bkr, ZH, aeR, JI, aW, aoo, Ls, hl, LZ, Qm, YG, PQ, g, as, eE, eF, uC, cm, Qt, zV, iP, bw, aAW, qJ, eM, nB, AM, nF, iA, vA, wB, cj, NI, dW, m, fy, cE, iu, Vj, dD, xu, vl, aEj, bkq, sw, zt, ht, NT, aBx, n, kv, fr, qN, auA, aje, asS, eW, El, ft, no, jz, Yc, bkm, ec, i, Nn, gd, lG, By, dL, ey, hB, Ha, Dg, asp, RT, CN, MD, As, hQ, Vt, ack, aq, kp, fe, auC, op, lx, KT, bkn, W, GU, dB, hk, eD, ajn, hL, gw, uz, hR, at, it, B, bL, eR, Dh, fG, WT, jx, aA, cz, fF, hy, v, yO, asA, bX, bkj, q, eA, qH, XX, Mg, mj, anA, lg, bjA, nv, Qg, qp, BJ, aT, kf, cy, anz, an, rz, ae, ne, bkl, dl, amx, s, ND, nO, oV, Y, ev, LO, iU, r, lo, tu, en, bu, cl, t, apx, bkp, pb, ik, bN, wA, tR, nM, kP, c, Dy, ac, wO, ll, nG, gX, WU, adR, CZ, dy, bko, anD, im, bt, ao, hS, On, hl, aD, jN, uO, jh, Hn, bjW, Fb, eq, jE, aml, ad, wD, hX, dP, av, iC, hd, F, ib, py, xl, yt, db, NS, tT, Sr, bj, Dj, hP, ji, ako, nJ, Yi, d, vj, ff, eH, cr, dN, tv, and, aF, hp, Fq, Pd, iR, bz, LB, es, D, gk, pu, arY, TA, kg, bE, Ew, AR, wz, Hs, adg, lC, X, y); #c1(EGF) c2(NP_001954) c3(pro-epidermal growth factor isoform 1 preproprotein) c4(122559) c5(122560, 122561, 122562, 122563, 122564, 122565, 122566) c6( )c7(ID, gh, G, T, bM, aC, pf, hV, ea, if, e, al, bO, eb, ajp, bB, axk, ahX, lR, ci, fd, aY, ip, x, Vp, bv, Na, aE, rJ, pX, aeO, bki, dV, auB, iv, ja, K, Nj, hu, bkk, eL, aa, ly, xQ, yo, bkr, ZH, aeR, JI, aW, aoo, Ls, hl, LZ, Qm, YG, PQ, g, as, eE, eF, uC, cm, Qt, zV, iP, bw, aAW, qJ, eM, nB, AM, nF, iA, vA, wB, cj, NI, dW, m, fy, cE, iu, Vj, dD, xu, vl, aEj, bkq, sw, zt, ht, NT, aBx, n, kv, fr, qN, auA, aje, asS, eW, El, ft, no, jz, Yc, bkm, ec, i, Nn, gd, lG, By, dL, ey, hB, Ha, Dg, asp, RT, CN, MD, As, hQ, Vt, ack, aq, kp, fe, auC, op, lx, KT, bkn, W, GU, dB, hk, eD, ajn, hL, gw, uz, hR, at, it, B, bL, eR, Dh, fG, WT, jx, aA, cz, fF, hy, v, yO, asA, bX, bkj, q, eA, qH, XX, Mg, mj, anA, lg, bjA, nv, Qg, qp, BJ, aT, kf, cy, anz, an, rz, ae, ne, bkl, dl, amx, s, ND, nO, oV, Y, ev, LO, il, r, lo, tu, en, bu, cl, t, apx, bkp, pb, ik, bN, wA, tR, nM, kP, c, Dy, ac, wl, ll, nG, gX, WU, adR, CZ, dy, bko, anD, im, bt, ao, hS, On, hl, aD, jN, uO, jh, Hn, bjW, Fb, eq, jE, aml, ad, wD, hX, dP, av, iC, hd, F, ib, py, xl, yt, db, NS, tT, Sr, bj, Dj, hP, ji, ako, nJ, Yi, d, vj, ff, eH, cr, dN, tv, and, aF, hp, Fq, Pd, iR, bz, LB, es, D, gk, pu, arY, TA, kg, bE, Ew, AR, wz, Hs, adg, lC, X, y); #c1(EGFL6) c2(NP_001161362) c3(epidermal growth factor-like protein 6 isoform 2 precursor) c4(65638) c5(65639, 65640, 65641, 65642, 65643, 65644) c6( )c7(bgw, t, aJl, m, ip); #c1(EGFL6) c2(NP_056322) c3(epidermal growth factor-like protein 6 isoform 1 precursor) c4(187061) c5(187062, 187063, 187064, 187065, 187066, 187067) c6( )c7(bgw, t, aJA, m, ip); #c1(EGFL7) c2(XP_011517068) c3(epidermal growth factor-like protein 7 isoform X1) c4(310562) c5(310563, 310564, 310565, 310566, 310567, 310568, 310569, 310570, 310571, 310572, 310573, 310574, 310575, 310576, 310577, 310578, 310579, 310580) c6( )c7(T, aYP, dG, eA, gd, aT, ey, H, B, qt, aW, oV, eZ, aYR, ys, bz, ac, aYa, wO, aYb, aYQ, Lp, Lq, in, X, L); #c1(EGFL8) c2(NP_085155) c3(epidermal growth factor-like protein 8 precursor) c4(219525, 219526, 219527, 219528) c5(219529, 219530, 219531, 219532, 219533, 219534, 219535, 219536, 219537, 219538, 219539, 219540, 219541, 219542, 219543, 219544, 219545, 219546, 219547, 219548, 219549, 219550, 219551, 219552, 219553, 219554, 219555, 219556, 219557, 219558, 219559, 219560, 219561, 219562, 219563, 219564, 219565, 219566, 219567, 219568, 219569, 219570, 219571, 219572, 219573, 219574, 219575, 219576, 219577, 219578, 219579, 219580, 219581, 219582, 219583, 219584, 219585, 219586, 219587, 219588, 219589, 219590, 219591, 219592, 219593, 219594, 219595, 219596, 219597, 219598, 219599, 219600, 219601, 219602, 219603, 219604, 219605, 219606, 219607, 219608, 219609, 219610, 219611, 219612, 219613, 219614, 219615, 219616, 219617, 219618, 219619) c6( )c7(T, A, W, eZ, r, q); #c1(EGFLAM) c2(NP_001192230) c3(pikachurin isoform 5 precursor) c4(82121, 82122) c5(82123, 82124, 82125, 82126, 82127, 82128, 82129, 82130, 82131, 82132) c6(82133, 82134) c7(cd, oe, xs); #c1(EGFLAM) c2(NP_689616) c3(pikachurin isoform 1 precursor) c4(243281, 243282, 243283, 243284) c5(243285, 243286, 243287, 243288, 243289, 243290, 243291, 243292, 243293) c6(243294, 243295) c7(cd, oe, xs); #c1(EGFLAM) c2(NP_877950) c3(pikachurin isoform 2) c4(256973) c5(256974, 256975, 256976, 256977, 256978, 256979, 256980, 256981, 256982, 256983, 256984, 256985, 256986, 256987, 256988, 256989, 256990, 256991, 256992, 256993, 256994, 256995, 256996, 256997) c6(256998, 256999, 257000, 257001, 257002, 257003) c7(cd, oe, xs); #c1(EGFLAM) c2(NP_877953) c3(pikachurin isoform 4) c4( )c5(257004, 257005, 257006, 257007, 257008, 257009, 257010, 257011, 257012, 257013, 257014, 257015, 257016, 257017, 257018, 257019, 257020, 257021, 257022, 257023, 257024, 257025, 257026, 257027) c6(257028, 257029, 257030, 257031, 257032, 257033, 257034) c7(cd, oe, xs); #c1(EGFR) c2(NP_005219) c3(epidermal growth factor receptor isoform a precursor) c4(154123, 154124, 154125, 154126, 154127, 154128) c5(154129, 154130, 154131, 154132, 154133, 154134, 154135, 154136, 154137, 154138, 154139, 154140, 154141, 154142, 154143, 154144, 154145, 154146, 154147, 154148, 154149, 154150, 154151, 154152, 154153, 154154, 154155, 154156, 154157, 154158, 154159, 154160, 154161, 154162, 154163, 154164) c6( )c7(gh, T, ea, bO, fd, ci, aY, bv, aE, fm, dE, ajv, auB, iv, hu, zy, bkk, azh, cp, aa, yo, iE, Ls, az, Az, QB, nY, bjk, zE, ahv, zV, iA, nB, avZ, bkv, cj, azg, cE, YK, aAz, iB, ht, RC, aj, cd, bao, auO, aoc, aex, bkP, aJb, pl, Yl, alJ, anP, MD, sp, Lh, fe, CO, iO, bkn, dB, eD, E, avr, bkE, aJJ, jv, Dh, fZ, aA, cz, bX, lu, AD, q, bkJ, Qg, qK, jj, ch, dl, iF, NE, nO, fO, cl, bkB, ig, Nk, RW, VA, nG, WU, aws, bt, rA, On, Mb, jN, Jy, jh, DE, Gn, ad, afb, hX, iC, yt, aFh, avs, lQ, wm, apw, bku, BO, ff, aeV, iV, bz, YR, mi, ag, azT, aed, ne, jr, iu, oO, bM, aBD, aEn, anl, bkx, bkF, if, bkl, bB, ip, anV, acO, ja, aeN, aNZ, bky, De, xQ, aeR, JI, fc, hl, Qq, kt, Im, NR, as, eE, eF, cm, Qt, qJ, bkD, vA, mx, NI, OD, Th, ik, Vj, dD, lT, wu, zt, n, oY, aje, aGR, Mo, jz, no, zw, ec, Do, Xe, gd, aAG, bkN, A, bkC, hT, PX, fJ, AD, GA, bP, GU, PI, hL, at, fl, Te, B, dA, v, Do, eJ, asG, RI, bjA, BJ, AJ, atZ, ae, jf, Qr, vT, ev, auc, acB, afs, bu, t, KQ, ck, aUc, kP, gX, anD, nZ, hl, Dd, eq, Fc, Tg, hd, py, db, ds, Ti, bkw, nJ, atX, d, dN, anW, Z, adj, bF, gk, arY, wz, gZ, QC, y, kS, aC, fR, azf, hV, al, axk, PB, Vp, bkA, xE, bW, pX, Qk, vg, K, eL, aEl, mQ, LZ, g, iP, AM, nF, Gs, U, PV, Hb, m, sw, zX, NT, qy, zP, asS, ft, lt, Tf, cU, vd, is, dx, ny, CN, ks, YF, cM, auC, Zj, Tk, JM, aEG, zl, it, avm, fG, jx, wo, acn, il, pd, eA, J, er, XX, lg, aAF, yg, bq, Y, LO, apK, r, en, ik, QG, c, np, dy, bkk, KY, LS, kh, id, bkL, auq, bS, bkM, OH, iJ, dP, io, hP, ji, Yi, anv, vj, eH, PA, cr, and, VI, hp, iR, KM, TA, Ew, AR, cP, G, jW, pf, WV, mF, Ou, DA, zQ, aAu, aW, eM, wB, uJ, gn, bkz, Xu, bks, aEj, Or, aBx, awr, fr, ct, qN, eW, co, Bl, hw, Yc, ic, h, dL, hB, Wr, asT, Xh, Lg, asp, aq, aeW, kp, bkH, op, aoO, Qy, bkQ, Qu, hk, gw, hR, ZZ, cn, bL, rG, yk, WT, hy, akW, bkG, nv, aoA, afQ, aT, Fx, Tj, af, Uf, s, iU, oV, aNi, bjl, OC, sS, nM, im, hS, aD, Fb, oH, mC, bkO, av, ib, Rq, oz, NS, fb, afe, qt, eZ, Ej, aF, Hp, LB, es, bkt, zr, bE, aBH, MC, adg, X); #c1(EGFR) c2(NP_958439) c3(epidermal growth factor receptor isoform b precursor) c4( )c5(262110, 262111, 262112, 262113, 262114, 262115) c6( )c7(gh, T, ea, bO, fd, ci, aY, bv, aE, fm, dE, ajv, auB, iv, hu, zy, bkk, azh, cp, aa, yo, iE, Ls, az, Az, QB, nY, bjk, zE, ahv, zV, iA, nB, avZ, bkv, cj, azg, cE, YK, aAz, iB, ht, RC, aj, cd, bao, auO, aoc, aeX, bkP, alb, pl, Yl, alJ, anP, MD, sp, Lh, fe, CO, iO, bkn, dB, eD, E, avr, bkE, aJJ, jv, Dh, fZ, aA, cz, bX, lu, AD, q, bkJ, Qg, qK, jj, ch, dl, iF, NE, nO, fO, cl, bkB, ig, Nk, RW, VA, nG, WU, aws, bt, rA, On, Mb, jN, Jy, jh, OE, Gn, ad, afb, hX, iC, yt, afh, avs, lQ, wm, apw, bku, BO, ff, aeV, iV, bz, YR, mi, ag, azT, aed, ne, jr, iu, oO, bM, aBD, aEn, anl, bkx, bkF, if, bkl, bB, ip, anV, aeO, ja, aeN, aNZ, bky, De, xQ, aeR, JI, fc, hl, Qq, kt, Im, NR, as, eE, eF, cm, lt, qJ, bkD, vA, mx, NI, OD, Th, ik, Vj, dD, lT, wu, zt, n, oY, aje, aGR, Mo, jz, no, zw, ec, Do, Xe, gd, aAG, bkN, A, bkC, hT, PX, fJ, AQ, GA, bP, GU, Pl, hL, at, fL, Te, B, dA, v, Qo, eJ, asG, RI, bjA, BJ, AJ, atZ, ae, jf, Or, vT, ev, auc, acB, afs, bu, t, KQ, ck, aUc, kP, gX, anD, nZ, hl, Dd, eq, Fc, Tg, hd, py, db, ds, Ti, bkw, nJ, atX, d, dN, anW, Z, adj, bF, gk, arY, wz, gZ, QC, y, kS, aC, fR, azf, hV, al, axk, PB, Vp, bkA, xE, bW, pX, Qk, vg, K, eL, aEl, ml, LZ, g, iP, AM, nF, Gs, U, PV, Hb, m, sw, zX, NT, qy, zP, asS, ft, lt, Tf, cU, vd, is, dx, ny, CN, ks, YF, cM, auC, Zj, Tk, JM, aEG, zl, it, avm, fG, jx, wo, acn, il, pd, eA, J, er, XX, lg, aAF, yg, bq, Y, LO, apK, r, en, ik, QG, c, np, dy, bkk, KY, LS, kh, id, bkL, auq, bS, bkM, OH, iJ, dP, io, hP, ji, Yi, anv, vj, eH, PA, cr, and, VI, hp, iR, KM, TA, Ew, AR, cP, G, jW, pf, WV, mF, Ou, DA, zQ, aAu, aW, eM, wB, uJ, gn, bkz, Xu, bks, aEj, Or, aBx, awr, fr, ct, qN, eW, co, Bl, hw, Yc, ic, h, dL, hB, Wr, asT, Xh, Lg, asp, aq, aeW, kp, bkH, op, aoO, Dy, bkQ, Qu, hk, gw, hR, ZZ, cn, bL, rG, yk, WT, hy, akW, bkG, nv, aoA, afQ, aT, Fx, Tj, af, Uf, s, il, oV, aNi, bjl, OC, sS, nM, im, hS, aD, Fb, oH, mC, bkO, av, ib, Rq, oz, NS, fb, afe, qt, eZ, Ej, aF, Hp, LB, es, bkt, zr, bE, aBH, MC, adg, X); #c1(EGFR) c2(NP_958440) c3(epidermal growth factor receptor isoform c precursor) c4(262116) c5(262117, 262118, 262119, 262120, 262121, 262122) c6( )c7(gh, T, ea, bO, fd, ci, aY, bv, aE, fm, dE, ajv, auB, iv, hu, zy, bkk, azh, cp, aa, yo, iE, Ls, az, Az, QB, nY, bjk, zE, ahv, zV, iA, nB, avZ, bkv, cj, azg, cE, YK, aAz, iB, ht, RC, aj, cd, bao, auO, aoc, aeX, bkP, alb, pl, Yl, alJ, anP, MD, sp, Lh, fe, CQ, iO, bkn, dB, eD, E, avr, bkE, aJJ, jv, Dh, fZ, aA, cz, bX, lu, AO, q, bkJ, Qg, qK, jj, ch, dl, iF, NE, nO, fO, cl, bkB, ig, Nk, RW, VA, nG, WU, aws, bt, rA, On, Mb, jN, Jy, jh, DE, Gn, ad, afb, hX, iC, yt, aFh, avs, lQ, wm, apw, bku, BO, ff, aeV, iV, bz, YR, mi, ag, azT, aed, ne, jr, iu, oO, bM, aBD, aEn, anl, bkx, bkF, if, bkl, bB, ip, anV, aeO, ja, aeN, aNZ, bky, De, xQ, aeR, JI, fc, hl, Qq, kt, Um, NR, as, eE, eF, cm, Qt, qJ, bkD, vA, mx, NI, OD, Th, ik, Vj, dD, lT, wu, zt, n, oY, aje, aGR, Mo, jz, no, zw, ec, Do, Xe, gd, aAG, bkN, A, bkC, HT, PX, fJ, AQ, GA, bP, GU, PI, hL, at, fL, Te, B, dA, v, Qo, eJ, asG, RI, bjA, BJ, AJ, atZ, ae, jf, Qr, vT, ev, auc, acB, afs, bu, t, KQ, ck, aUc, kP, gX, anD, nZ, hl, Dd, eq, Fc, Tg, hd, py, db, ds, Ti, bkw, nJ, atX, d, dN, anW, Z, adj, bF, gk, arY, wz, gZ, QC, y, kS, aC, fR, azf, hV, al, axk, PB, Vp, bkA, xE, bW, pX, Qk, vg, K, eL, aEl, mQ, LZ, g, iP, AM, nF, Gs, U, PV, Hb, m, sw, zX, NT, qy, zP, asS, ft, lt, Tf, cU, vd, is, dx, ny, CN, ks, YF, cM, auC, Zj, Tk, JM, aEG, zl, it, avm, fG, jx, wo, acn, il, pd, eA, J, er, XX, lg, aAF, yg, bq, Y, LO, apK, r, en, ik, QG, c, np, dy, bkk, KY, LS, kh, id, bkL, auq, bS, bkM, OH, iJ, dP, io, hP, ji, Yi, anv, vj, eH, PA, cr, and, VI, hp, iR, KM, TA, Ew, AR, cP, G, jW, pf, WV, mF, Ou, DA, zQ, aAu, aW, eM, wB, uJ, gn, bkz, Xu, bks, aEj, Or, aBx, awr, fr, ct, qN, eW, co, Bl, hw, Yc, ic, h, dL, hB, Wr, asT, Xh, Lg, asp, aq, aeW, kp, bkH, op, aol, By, bkQ, Qu, hk, gw, hR, ZZ, cn, bL, rG, yk, WT, hy, akW, bkG, nv, aoA, afQ, aT, Fx, Tj, af, Uf, s, il, oV, aNi, bjl, OC, sS, nM, im, hS, aD, Fb, oH, mC, bkO, av, ib, Rq, oz, NS, fb, afe, qt, eZ, Ej, aF, Hp, LB, es, bkt, zr, bE, aBH, MC, adg, X); #c1(EGFR) c2(NP_958441) c3(epidermal growth factor receptor isoform d precursor) c4(262123, 262124) c5(262125, 262126, 262127, 262128, 262129, 262130) c6( )c7(gh, T, ea, bO, fd, ci, aY, bv, aE, fm, dE, ajv, auB, iv, hu, zy, bkk, azh, cp, aa, yo, iE, Ls, az, Az, QB, nY, bjk, zE, ahv, zV, iA, nB, avZ, bkv, cj, azg, cE, YK, aAz, iB, ht, RC, aj, cd, bao, auO, aoc, aeX, bkP, aJb, pl, Yl, alJ, anP, MD, sp, Lh, fe, CQ, iO, bkn, dB, eD, E, avr, bkE, aJJ, jv, Dh, fZ, aA, cz, bX, lu, AO, q, bkJ, Qg, qK, jj, ch, dl, iF, NE, nO, fO, cl, bkB, ig, Nk, RW, VA, nG, WU, aws, bt, rA, On, Mb, jN, Jy, jh, DE, Gn, ad, afb, hX, iC, yt, afh, avs, lQ, wm, apw, bku, BO, ff, aeV, iV, bz, YR, mi, ag, azT, aed, ne, jr, iu, oO, bM, aBD, aEn, anl, bkx, bkF, if, bkl, bB, ip, anV, aeO, ja, aeN, aNZ, bky, De, xQ, aeR, JI, fc, hl, Qq, kt, Qm, NR, as, eE, eF, cm, Qt, qJ, bkD, vA, mx, NI, OD, Th, ik, Vj, dD, lT, wu, zt, n, oY, aje, aGR, Mo, jz, no, zw, ec, Do, Xe, gd, aAG, bkN, A, bkC, hT, PX, fJ, AQ, GA, bP, GU, PI, hL, at, fL, Te, B, dA, v, Do, eJ, asG, RI, bjA, BJ, AJ, atZ, ae, jf, Or, vT, ev, auc, acB, afs, bu, t, KQ, ck, aUc, kP, gX, anD, nZ, hl, Dd, eq, Fc, Tg, hd, py, db, ds, Ti, bkw, nJ, atX, d, dN, anW, Z, adj, bF, gk, arY, wz, gZ, QC, y, kS, aC, fR, azf, hV, al, axk, PB, Vp, bkA, xE, bW, pX, Qk, vg, K, eL, aEl, mQ, LZ, g, iP, AM, nF, Gs, U, PV, Hb, m, sw, zX, NT, qy, zP, asS, ft, lt, Tf, cU, vd, is, dx, ny, CN, ks, YF, cM, auC, Zj, Tk, JM, aEG, zl, it, avm, fG, jx, wo, acn, il, pd, eA, J, er, XX, lg, aAF, yg, bq, Y, LO, apK, r, en, ik, QG, c, np, dy, bkk, KY, LS, kh, id, bkL, auq, bS, bkM, OH, iJ, dP, io, hP, ji, Yi, anv, vj, eH, PA, cr, and, VI, hp, iR, KM, TA, Ew, AR, cP, G, jW, pf, WV, mF, Ou, DA, zl, aAu, aW, eM, wB, uJ, gn, bkz, Xu, bks, aEj, Or, aBx, awr, fr, ct, qN, eW, co, Bl, hw, Yc, ic, h, dL, hB, Wr, asT, Xh, Lg, asp, aq, aeW, kp, bkH, op, aoO, Qy, bkQ, Qu, hk, gw, hR, ZZ, cn, bL, rG, yk, WT, hy, akW, bkG, nv, aoA, afQ, aT, Fx, Tj, af, Uf, s, il, oV, aNi, bjl, OC, sS, nM, im, hS, aD, Fb, OH, mC, bkO, av, ib, Rq, oz, NS, fb, afe, qt, eZ, Ej, af, Hp, LB, es, bkt, zr, bE, aBH, MC, adg, X); #c1(EGLN1) c2(NP_071334) c3(egl nine homolog 1) c4(211057) c5(211058, 211059, 211060, 211061, 211062, 211063, 211064) c6( )c7(if, bB, fr, ci, qN, bkU, ai, oT, py, hw, oW, aT, ji, K, sR, bkR, eu, cp, bkS, aW, Y, ff, en, cl, ik, uR, hL, bkT, anx, y, sZ, wS, bkV); #c1(EGLN1) c2(XP_005273224) c3(egl nine homolog I isoform X2) c4(284372) c5(284373, 284374, 284375, 284376, 284377, 284378, 284379) c6( )c7(if, bB, fr, ci, qN, bkU, ai, oT, py, hw, oW, aT, ji, K, sR, bkR, eu, cp, bkS, aW, Y, ff, en, cl, ik, uR, hL, bkT, anx, y, sZ, wS, bkV); #c1(EGLN2) c2(NP_444274) c3(egl nine homolog 2) c4(230229) c5(230230, 230231, 230232, 230233, 230234, 230235, 230236, 230237, 230238, 230239, 230240) c6( )c7(T, Uc, eM, qt, aW, aqh, WA, z, Gv, aT, kH, at, fe, fr, eA, ji, hf, aH); #c1 (EGLN3) c2(NP_071356) c3(egl nine homolog 3) c4(211187) c5(211188, 211189, 211190, 211191, 211192, 211193, 211194, 211195, 211196, 211197) c6( )c7(T, aw, aA, bB, fr, ci, q, ad, eA, Hr, oT, cc, aE, aT, cy, rO, ji, sR, ae, aH, bA, aW, r, aq, kp, aF, iV, cm, iK, ac, c, z, go, tV, B, VB); #c1(EGR1) c2(NP_001955) c3(early growth response protein 1) c4(122567, 122568, 122569) c5(122570, 122571, 122572, 122573, 122574, 122575, 122576, 122577, 122578, 122579, 122580, 122581, 122582, 122583, 122584, 122585, 122586, 122587, 122588) c6( )c7(gh, T, os, bM, Js, ea, if, fd, bB, lR, ci, aHR, gH, fm, K, hu, oi, eL, aa, eG, dH, HI, aW, az, fc, aBd, g, as, hJ, aKj, eF, cm, bw, eM, nB, iA, acS, iT, vA, Hb, m, iu, dD, wu, ht, n, aj, oY, awr, fr, qN, jw, fK, br, eW, FN, lG, h, dL, pi, apc, oS, RT, fe, aq, op, hq, ar, kT, Gq, dB, Fi, Ei, Be, hL, at, bm, zB, it, B, Jo, fG, aA, ol, v, j, q, J, er, eA, mj, fh, aT, cy, dz, rO, ae, af, eu, Y, r, cu, lo, en, cl, t, ik, pb, ck, iM, ac, wD, dy, aws, bt, qb, ha, ul, bs, jh, oZ, bS, dG, eq, Fb, iJ, ad, aHF, ib, py, Ot, oW, wm, NM, d, je, et, ff, gK, eH, gE, aF, KM, bz, Hv, LB, es, bE, Ny, HN, aS, ra, lC, xg, X, y); #c1(EGR2) c2(NP_001129650) c3(E3 SUMO-protein ligase EGR2 isoform a) c4(52095, 52096) c5(52097, 52098, 52099, 52100, 52101, 52102, 52103, 52104, 52105, 52106, 52107) c6( )c7(Pt, xF, bkY, ha, agU, aA, j, fu, bX, EA, il, lR, mC, hX, no, bK, bU, oi, af, dx, HA, eZ, aF, bQ, t, cm, pb, op, bN, bkZ, Qi, ql, bkW, iT, lq, vA, wR, fl, y, bkX); #c1(EGR2) c2(XP_011537729) c3(E3 SUMO-protein ligase EGR2 isoform X2) c4(335280) c5(335281, 335282, 335283, 335284, 335285, 335286, 335287) c6( )c7(Pt, xF, bkY, ha, agU, aA, j, fu, bX, EA, il, lR, mC, hX, no, bK, bU, oi, af, dx, HA, eZ, aF, bQ, t, cm, pb, op, bN, bkZ, Di, ql, bkW, iT, lq, vA, wR, fL, y, bkX); #c1(EGR2) c2(XP_011537731) c3(E3 SUMO-protein ligase EGR2 isoform X1) c4( )c5(335288, 335289, 335290, 335291, 335292) c6( )c7(Pt, xF, bkY, ha, agU, aA, j, fu, bX, EA, il, lR, mC, hX, no, bK, bU, oi, af, dx, HA, eZ, af, bl, t, cm, pb, op, bN, bkZ, Qi, ql, bkW, iT, lq, vA, wR, fl, y, bkX); #c1(EGR3) c2(NP_001186810) c3 (early growth response protein 3 isoform 3) c4( )c5(78619, 78620, 78621, 78622, 78623, 78624) c6( ) c7(T, iH, ha, n, j, ci, lR, mC, hX, kH, aT, oi, he, af, je, aW, eZ, pb, iM, lq, cj, fl, cB); #c1(EGR3) c2(NP_004421) c3(early growth response protein 3 isoform 1) c4(146240, 146241) c5(146242, 146243, 146244, 146245, 146246, 146247, 146248) c6( )c7(T, iH, ha, n, j, ci, lR, mC, hX, kH, aT, oi, he, af, je, aW, eZ, pb, iM, lq, cj, fL, cB); #c1(EGR3) c2(XP_005273483) c3(early growth response protein 3 isoform X1) c4(284605) c5(284606, 284607, 284608, 284609, 284610) c6( )c7(T, iH, ha, n, j, ci, lR, mC, hX, KH, aT, oi, he, af, je, aW, eZ, pb, iM, lq, cj, fL, cB); #c1(EGR3) c2(XP_011542731) c3(early growth response protein 3 isoform X3) c4( )c5(341315) c6( )c7(T, iH, ha, n, j, ci, lR, mC, hX, KH, aT, oi, he, af, je, aW, eZ, pb, iM, lq, cj, fL, cB); #c1(EGR4) c2(NP_001956) c3(early growth response protein 4) c4(122589, 122590) c5(122591, 122592, 122593, 122594, 122595, 122596, 122597, 122598, 122599, 122600, 122601, 122602, 122603) c6( )c7(oi, bL, gU, ND); #c1 (EHBP1) c2(NP_001136087) c3(EH domain-binding protein 1 isoform 3) c4(54284, 54285) c5(54286, 54287, 54288, 54289, 54290, 54291, 54292, 54293, 54294, 54295, 54296, 54297, 54298, 54299, 54300, 54301) c6( )c7(cm, ae, ad, bla); #c1(EHBP1) c2(XP_005264284) c3(EH domain-binding protein 1 isoform X2) c4(278769) c5(278770, 278771, 278772, 278773, 278774, 278775, 278776, 278777, 278778, 278779) c6( )c7(cm, ae, ad, bla); #c1(EHBP1) c2(XP_005264285) c3(EH domain-binding protein 1 isoform X7) c4( )c5(278780, 278781, 278782, 278783, 278784, 278785, 278786, 278787, 278788, 278789, 278790, 278791) c6( )c7(cm, ae, ad, bla); #c1(EHBP1) c2(XP_011531017) c3(EH domain-binding protein 1 isoform X1) c4(326978) c5(326979, 326980, 326981, 326982, 326983, 326984) c6( )c7(cm, ae, ad, bla); #c1(EHBP1) c2(XP_011531018) c3(EH domain-binding protein 1 isoform X3) c4(326985) c5( ) c6( )c7(cm, ae, ad, bla); #c1(EHBP1) c2(XP_011531020) c3(EH domain-binding protein 1 isoform X6) c4(326986) c5( )c6( )c7(cm, ae, ad, bla); #c1(EHD1) c2(NP_001269374) c3(EH domain-containing protein 1 isoform 2) c4(101317, 101318, 101319, 101320, 101321) c5(101322, 101323, 101324, 101325, 101326, 101327, 101328, 101329, 101330, 101331, 101332, 101333, 101334, 101335, 101336, 101337, 101338, 101339, 101340, 101341, 101342, 101343, 101344, 101345) c6( )c7(bL, cc, aZ, Y, Be, V, bN); #c1(EHD1) c2(NP_006786) c3(EH domain-containing protein 1 isoform 1) c4( )c5(168059, 168060, 168061, 168062, 168063, 168064, 168065, 168066, 168067, 168068) c6( )c7(bL, cc, aZ, Y, Be, V, bN); #c1(EHD2) c2(NP_055416) c3(EH domain-containing protein 2) c4(180987, 180988, 180989, 180990, 180991, 180992) c5(180993, 180994, 180995, 180996, 180997, 180998, 180999, 181000, 181001, 181002, 181003, 181004, 181005, 181006, 181007, 181008, 181009, 181010, 181011, 181012, 181013, 181014, 181015, 181016, 181017, 181018, 181019, 181020, 181021, 181022, 181023, 181024, 181025, 181026, 181027, 181028) c6( )c7(bL, bM, NK, T, dD, acz, aAz, ea, agU, ha, cV, jh, bgj, ajM, eq, fu, fr, bgR, q, eA, hX, x, fx, alu, oz, Dq, blb, qh, yg, aT, aU, h, K, akr, dx, HO, aa, jf, iF, aW, Y, xs, eZ, bfu, r, fe, lo, cm, bz, bN, aBs, ck, es, y, Bv, eM, iA, dB, Qu, ag, aiM, lO, k, pJ, bt, ao); #c1(EHD3) c2(NP_055415) c3(EH domain-containing protein 3) c4(180974, 180975) c5(180976, 180977, 180978, 180979, 180980, 180981, 180982, 180983, 180984, 180985, 180986) c6( )c7(bL, bM, T, aGn, pf, bO, GC, oY, bS, j, ct, ci, v, yU, lu, fw, cd, br, eA, aE, pX, h, yZ, aNU, dz, jj, an, blc, apc, mR, Y, eZ, dq, bld, jg, Z, bw, adz, cT, dB, gZ, afW, bt, kS); #c1(EHD3) c2(XP_011531108) c3(EH domain-containing protein 3 isoform X1) c4( )c5(327091) c6( )c7(bL, bM, T, aGn, pf, bO, GC, oY, bS, j, ct, ci, v, yU, lu, fw, cd, br, eA, aE, pX, h, yZ, aNU, dz, jj, an, blc, apc, mR, Y, eZ, dq, bld, jg, Z, bw, adz, cT, dB, gZ, afW, bt, kS); #c1(EHD4) c2(NP_644670) c3(EH domain-containing protein 4) c4(238483) c5(238484, 238485, 238486, 238487, 238488, 238489) c6( )c7(bL, T, Y, jN, ap, uY, v, bt, bA); #c1(EHF) c2(NP_001193545) c3(ETS homologous factor isoform 1 precursor) c4(82256, 82257) c5(82258, 82259, 82260, 82261, 82262, 82263) c6( )c7(bM, T, xQ, ac, aT, agf, ci, m, ae, ad, hS); #c1 (EHF) c2(XP_005252918) c3(ETS homologous factor isoform X5) c4(272067) c5(272068) c6( )c7(bM, T, xQ, ac, aT, agF, ci, m, ae, ad, hS); #c1(EHF) c2(XP_005252919) c3(ETS homologous factor isoform X2) c4( )c5(272069, 272070, 272071, 272072, 272073) c6( )c7(bM, T, xQ, ac, aT, agF, ci, m, ae, ad, hS); #c1(EHF) c2(XP_011518285) c3(ETS homologous factor isoform X4) c4( )c5(312309, 312310, 312311, 312312) c6( )c7(bM, T, xQ, ac, al, agF, ci, m, ae, ad, hS); #c1(EHF) c2(XP_011518287) c3(ETS homologous factor isoform X1) c4( )c5(312313, 312314, 312315, 312316, 312317, 312318, 312319, 312320, 312321, 312322, 312323) c6( )c7(bM, T, xQ, ac, aT, agF, ci, m, ae, ad, hS); #c1(EHHADH) c2(NP_001159887) c3(peroxisomal bifunctional enzyme isoform 2) c4( )c5(64887, 64888, 64889, 64890, 64891) c6( )c7(blf, af, bE, ble, X, hl, Zn, oh, bK, ci, C, oi, qO, lB, dF, B); #c1(EHHADH) c2(NP_001957) c3(peroxisomal bifunctional enzyme isoform 1) c4(122604, 122605, 122606) c5(122607, 122608, 122609, 122610, 122611, 122612, 122613, 122614, 122615, 122616, 122617, 122618, 122619) c6( )c7(blf, af, bE, ble, X, hl, Zn, oh, bK, ci, C, oi, qO, lB, dF, B); #c1(EHMT1) c2(NP_001138999) c3(histone-lysine N-methyltransferase EHMT1 isoform 2) c4(58590, 58591) c5(58592, 58593, 58594, 58595, 58596) c6( )c7(bL, dA, e, aj, dG, j, v, nA, J, mj, blh, aT, jy, blg, he, hm, jf, A, r, jb, W, sv, ra); #c1 (EHMT1) c2(NP_079033) c3(histone-lysine N-methyltransferase EHMT1 isoform 1) c4(216693) c5(216694, 216695, 216696, 216697, 216698) c6( )c7(bL, dA, e, aj, dG, j, v, nA, J, mj, blh, aT, jy, blg, he, hm, jf, A, r, jb, W, sv, ra); #c1(EHMT1) c2(XP_005266167) c3(histone-lysine N-methyltransferase EHMT1 isoform X7) c4( )c5(279945, 279946, 279947, 279948, 279949) c6( )c7(bL, dA, e, aj, dG, j, v, nA, J, mj, blh, aT, jy, blg, he, hm, jf, A, r, jb, W, sv, ra); #c1(EHMT1) c2(XP_006717351) c3(histone-lysine N-methyltransferase EHMT1 isoform X5) c4(290963) c5(290964, 290965) c6( )c7(bL, dA, e, aj, dG, j, v, nA, J, mj, blh, aT, jy, blg, he, hm, jf, A, r, jb, W, sv, ra); #c1(EHMT1) c2(XP_011517329) c3(histone-lysine N-methyltransferase EHMT1 isoform X9) c4( ) c5(310997, 310998, 310999, 311000, 311001, 311002) c6( )c7(bL, dA, e, aj, dG, j, v, nA, J, mj, blh, aT, jy, blg, he, hm, jf, A, r, jb, W, sv, ra); #c1 (EHMT1) c2(XP_011517330) c3(histone-lysine N-methyltransferase EHMT1 isoform X10) c4( )c5(311003) c6( )c7(bL, dA, e, aj, dG, j, v, nA, J, mj, blh, aT, jy, blg, he, hm, jf, A, r, jb, W, sv, ra); #c1(EHMT2) c2(NP_001276342) c3(histone-lysine N-methyltransferase EHMT2 isoform c) c4(108142, 108143) c5(108144, 108145, 108146, 108147, 108148, 108149, 108150, 108151, 108152, 108153, 108154, 108155, 108156, 108157, 108158, 108159, 108160, 108161, 108162, 108163, 108164, 108165, 108166, 108167, 108168, 108169, 108170, 108171, 108172, 108173, 108174, 108175, 108176, 108177, 108178, 108179, 108180, 108181, 108182, 108183, 108184, 108185, 108186, 108187, 108188, 108189, 108190, 108191, 108192, 108193, 108194, 108195, 108196, 108197, 108198, 108199, 108200, 108201, 108202) c6( )c7(av, T, W, eZ, aT, r, cu, sv, ci, jE, jj, hF, V, kS, q, jb); #c1(EHMT2) c2(NP_006700) c3(histone-lysine N-methyltransferase EHMT2 isoform a) c4(167242, 167243, 167244) c5( ) c6( )c7(av, T, W, eZ, aT, r, cu, sv, ci, jE, jj, hF, V, kS, q, jb); #c1(EHMT2) c2(NP_079532) c3(histone-lysine N-methyltransferase EHMT2 isoform b) c4(218618, 218619, 218620) c5(218621, 218622, 218623, 218624, 218625, 218626, 218627, 218628, 218629, 218630, 218631, 218632, 218633, 218634, 218635, 218636, 218637, 218638, 218639, 218640, 218641, 218642, 218643, 218644) c6( )c7(av, T, W, eZ, aT, r, cu, sv, ci, jE, jj, hF, V, kS, q, jb); #c1(EHMT2) c2(XP_005248881) c3(histone-lysine N-methyltransferase EHMT2 isoform X2) c4(268711, 268712) c5(268713, 268714, 268715, 268716, 268717, 268718, 268719, 268720, 268721, 268722, 268723, 268724, 268725, 268726, 268727, 268728, 268729, 268730, 268731, 268732, 268733, 268734, 268735, 268736, 268737, 268738, 268739, 268740, 268741, 268742, 268743, 268744, 268745, 268746, 268747, 268748, 268749, 268750, 268751, 268752, 268753) c6( )c7(av, T, W, eZ, aT, r, cu, sv, ci, jE, jj, hF, V, kS, q, jb); #c1(EHMT2) c2(XP_005274890) c3(histone-lysine N-methyltransferase EHMT2 isoform X2) c4(285511) c5(285512, 285513, 285514, 285515, 285516, 285517, 285518, 285519, 285520, 285521, 285522, 285523, 285524, 285525, 285526, 285527, 285528, 285529) c6( )c7(av, T, W, eZ, aT, r, cu, sv, ci, jE, jj, hF, V, kS, q, jb); #c1(EHMT2) c2(XP_006715039) c3(histone-lysine N-methyltransferase EHMT2 isoform X4) c4(288535, 288536) c5(288537, 288538, 288539, 288540, 288541, 288542, 288543, 288544, 288545, 288546, 288547, 288548, 288549, 288550, 288551, 288552, 288553, 288554, 288555, 288556, 288557, 288558, 288559, 288560, 288561) c6( )c7(av, T, W, eZ, aT, r, cu, sv, ci, jE, jj, hF, V, kS, q, jb); #c1(EI24) c2(XP_011541371) c3(etoposide-induced protein 2.4 homolog isoform X1) c4(339983) c5(339984, 339985, 339986, 339987, 339988, 339989, 339990) c6( )c7(T, bP, aW, yk, aT, n, ci, qN, ad); #c1(EI24) c2(XP_011541372) c3(etoposide-induced protein 2.4 homolog isoform X2) c4(339991) c5(339992, 339993, 339994, 339995, 339996) c6( )c7(T, bP, aW, yk, aT, n, ci, qN, ad); #c1(EID1) c2(NP_055150) c3(EP300-interacting inhibitor of differentiation 1) c4(179463) c5(179464, 179465, 179466, 179467, 179468, 179469) c6( )c7(ib, nO, af, FT, hS); #c1(EIF1) c2(NP_005792) c3(eukaryotic translation initiation factor 1) c4(159369) c5(159370, 159371, 159372, 159373, 159374, 159375) c6( )c7(dP, bM, hX, al, eZ, ci, fD, be, B, vi); #c1 (EIF1AD) c2(NP_001229411) c3(probable RNA-binding protein EIF1AD) c4(84363, 84364, 84365, 84366, 84367) c5(84368, 84369, 84370, 84371, 84372, 84373, 84374, 84375, 84376, 84377, 84378, 84379, 84380, 84381, 84382, 84383, 84384, 84385, 84386, 84387, 84388, 84389, 84390, 84391, 84392, 84393, 84394, 84395, 84396, 84397, 84398, 84399, 84400, 84401, 84402, 84403, 84404, 84405, 84406, 84407, 84408, 84409) c6( )c7(Y); #c1(EIF1AX) c2(NP_001403) c3(eukaryotic translation initiation factor 1A, X-chromosomal) c4(117608, 117609) c5(117610, 117611, 117612, 117613, 117614, 117615, 117616, 117617, 117618, 117619, 117620, 117621) c6( )c7(Y, iP); #c1(EIF1AY) c2(NP_001265541) c3(eukaryotic translation initiation factor 1A, Y-chromosomal isoform 2) c4(98779) c5(98780, 98781, 98782, 98783, 98784, 98785) c6( )c7(vH, bN); #c1(EIF1AY) c2(NP_004672) c3(eukaryotic translation initiation factor 1A, Y-chromosomal isoform 1) c4(148723) c5(148724, 148725, 148726, 148727, 148728, 148729) c6( )c7(vH, bN); #c1(EIF2A) c2(NP_114414) c3(eukaryotic translation initiation factor 2A) c4(222668) c5(222669, 222670, 222671, 222672, 222673, 222674) c6( )c7(jx, fd, fe, lG, j, K, cM); #c1(EIF2A) c2(XP_011511525) c3(eukaryotic translation initiation factor 2A isoform X1) c4(302174) c5(302175, 302176, 302177, 302178, 302179, 302180) c6( )c7(jx, fd, fe, lG, j, K, cM); #c1(EIF2AK1) c2(NP_055228) c3(eukaryotic translation initiation factor 2-alpha kinase 1 isoform a) c4(179963, 179964) c5(179965, 179966, 179967, 179968, 179969, 179970, 179971, 179972, 179973, 179974, 179975, 179976) c6( )c7(aCr, T, aCq, ac, Jr, aW, NT, cr, aT, lQ, K, hu, wD); #c1(EIF2AK2) c2(NP_001129124) c3(interferon-induced, double-stranded RNA-activated protein kinase isoform b) c4(50845, 50846, 50847) c5(50848, 50849, 50850, 50851, 50852, 50853, 50854, 50855, 50856, 50857, 50858, 50859, 50860, 50861, 50862, 50863, 50864, 50865) c6( )c7(Dw, T, os, eW, fR, e, ht, al, jx, aw, jh, oZ, j, ci, wy, hF, V, mC, aR, ad, hX, qC, av, x, aaF, F, hd, aE, db, rJ, ds, aT, cU, h, hn, K, hu, af, eu, afu, d, aW, Y, ff, eH, eZ, pR, lo, aq, CO, en, eF, t, cm, bz, dS, ck, rR, ac, c, eM, U, cj, rK, k, fL, hh, cB, B, EB, L); #c1(EIF2AK2) c2(NP_002750) c3(interferon-induced, double-stranded RNA-activated protein kinase isoform a) c4(131690, 131691) c5(131692, 131693, 131694, 131695, 131696, 131697, 131698, 131699, 131700, 131701, 131702, 131703, 131704, 131705, 131706, 131707) c6( )c7(Dw, T, os, eW, fR, e, ht, al, jx, aw, jh, oZ, j, ci, wy, hF, V, mC, aR, ad, hX, qC, av, x, aaF, F, hd, aE, db, rJ, ds, aT, cU, h, hn, K, hu, af, eu, afu, d, aW, Y, ff, eH, eZ, pR, lo, aq, CO, en, eF, t, cm, bz, dS, ck, rR, ac, c, eM, U, cj, rK, k, fL, hh, cB, B, EB, L); #c1(EIF2AK3) c2(NP_004827) c3(eukaryotic translation initiation factor 2-alpha kinase 3 precursor) c4(150104, 150105) c5(150106, 150107, 150108, 150109, 150110, 150111, 150112, 150113, 150114, 150115, 150116, 150117) c6( )c7(T, fR, jx, aj, j, eW, eA, J, F, mh, aT, cy, K, li, A, aW, fc, bo, TF, act, cP, cB); #c1(EIF2AK4) c2(NP_001013725) c3(eukaryotic translation initiation factor 2-alpha kinase 4) c4(25716, 25717, 25718) c5(25719, 25720, 25721, 25722, 25723, 25724, 25725, 25726, 25727, 25728, 25729, 25730, 25731, 25732, 25733, 25734, 25735, 25736) c6( )c7(ID, ly, blj, beH, beJ, T, bli, oV, Zs, oj, afB, lo, K, cb, lC, rl, lx, afu); #c1(EIF2AK4) c2(XP_011519902) c3(eukaryotic translation initiation factor 2-alpha kinase 4 isoform X3) c4(314689) c5(314690, 314691, 314692, 314693, 314694, 314695, 314696) c6( )c7(ID, ly, blj, beH, beJ, T, bli, oV, Zs, oj, afB, lo, K, cb, lC, rL, lx, afu); #c1(EIF2BI) c2(NP_001405) c3(translation initiation factor elf-28 subunit alpha) c4(117622) c5(117623, 117624, 117625, 117626, 117627, 117628) c6( )c7(aR, hb, il, U, bK, K, blk, cb, zZ, bll, dr); #c1(EIF282) c2(NP_055054) c3(translation initiation factor eIF-28 subunit beta) c4(178623) c5(178624, 178625, 178626, 178627, 178628, 178629) c6( )c7(aR, hb, il, U, bK, K, blk, cb, zZ, bll, CW, dr); #c1(EIF283) c2(XP_011540698) c3(translation initiation factor elf-28 subunit gamma isoform X1) c4(339269) c5( )c6( )c7(bll, blk); #c1(EIF284) c2(NP_001029288) c3(translation initiation factor elf-28 subunit delta isoform 2) c4(30781) c5(30782, 30783, 30784, 30785, 30786, 30787) c6( )c7(aR, kl, hb, il, ag, U, v, bK, K, blk, cb, zZ, bll, iu, dr); #c1(EIF284) c2(NP_056451) c3(translation initiation factor eIF-28 subunit delta isoform 3) c4(187602, 187603) c5(187604, 187605, 187606, 187607, 187608, 187609, 187610, 187611, 187612, 187613, 187614, 187615) c6( )c7(aR, kl, hb, il, ag, U, v, bK, K, blk, cb, zZ, bll, iu, dr); #c1(EIF284) c2(NP_751945) c3(translation initiation factor eIF-28 subunit delta isoform 1) c4(248566) c5(248567, 248568, 248569, 248570, 248571, 248572) c6( )c7(aR, kl, hb, il, ag, U, v, bK, K, blk, cb, zZ, bll, iu, dr); #c1(EIF2B5) c2(NP_003898) c3(translation initiation factor elf-28 subunit epsilon) c4(141947) c5(141948, 141949, 141950, 141951, 141952, 141953) c6( ) c7(blm, T, abV, ha, K, blk, V, bll, iu); #c1(EIF285) c2(XP_011511567) c3(translation initiation factor elf-28 subunit epsilon isoform X1) c4(302302) c5(302303, 302304, 302305, 302306, 302307, 302308) c6( )c7(blm, T, abV, ha, K, blk, V, bll, iu); #c1(EIF2SI) c2(NP_004085) c3(eukaryotic translation initiation factor 2 subunit 1) c4(143428, 143429, 143430) c5(143431, 143432, 143433, 143434, 143435, 143436, 143437, 143438, 143439, 143440, 143441, 143442, 143443, 143444, 143445, 143446, 143447, 143448, 143449, 143450, 143451, 143452) c6( )c7(T, al, jx, eX, aj, jE, nA, ci, il, eW, J, hX, db, HC, aT, aAn, cy, h, eV, ey, K, af, li, s, aW, ff, eH, fc, fe, aq, lo, en, pm, eF, QK, iR, bz, XT, gk, c, nB, bE, au, U, TF, cB, blj); #c1(EIF2S2) c2(NP_003899) c3(eukaryotic translation initiation factor 2 subunit 2) c4(141954) c5(141955, 141956, 141957, 141958, 141959, 141960) c6( )c7(al, jx, aj, nA, blk, aR, J, hb, il, aT, h, cy, bK, K, cb, aW, ff, aq, lo, en, iR, QK, zZ, dr, gk, c, au, U, TF, bll, blj); #c1(EIF2S3) c2(NP_001406) c3(eukaryotic translation initiation factor 2 subunit 3) c4(117629, 117630, (17631) c5(117632, 117633, 117634, 117635, 117636, 117637, 117638, 117639, 117640, 117641, 117642, 117643, 117644, 117645, 117646, 117647, 117648, 117649) c6( )c7(al, jx, aj, nA, aR, J, aT, h, cy, K, aW, ff, aq, lo, en, iR, QK, gk, c, au, U, TF, y, blj); #c1(EIF3A) c2(NP_003741) c3(eukaryotic translation initiation factor 3 subunit A) c4(140553, 140554) c5(140555, 140556, 140557, 140558, 140559, 140560, 140561, 140562, 140563, 140564, 140565, 140566, 140567, 140568) c6( )c7(T, jx, ci, eA, ad, x, aT, bl, Wq, sj, li, d, iU, aW, ff, eH, aF, as, en, eF, t, g, bz, ck, eM, dB, m, k, bt, iu); #c1(EIF3B) c2(NP_001032360) c3(eukaryotic translation initiation factor 3 subunit B) c4(31248, 31249, 31250) c5(31251, 31252, 31253, 31254, 31255, 31256, 31257, 31258, 31259, 31260, 31261, 31262, 31263, 31264, 31265, 31266, 31267, 31268, 31269, 31270, 31271, 31272) c6( )c7(ov, T, eM, eL, HK, h, dl, ci, cm, ae, B, ad); #c1(EIF3C) c2(NP_001254503) c3(eukaryotic translation initiation factor 3 subunit C isoform a) c4(94554, 94555) c5(94556, 94557, 94558, 94559, 94560, 94561, 94562, 94563, 94564, 94565, 94566, 94567, 94568, 94569, 94570, 94571, 94572, 94573, 94574, 94575) c6( )c7(wu, Ew, wm); #c1(EIF3C) c2(NP_001273407) c3(eukaryotic translation initiation factor 3 subunit C isoform b) c4(104832, 104833) c5(104834, 104835, 104836, 104837, 104838, 104839, 104840, 104841, 104842, 104843, 104844, 104845) c6( )c7(wu, Ew, wm); #c1(EIF3E) c2(NP_001559) c3(eukaryotic translation initiation factor 3 subunit E) c4(118966) c5(118967, 118968, 118969, 118970, 118971, 118972) c6( )c7(av, T, eM, aW, aT, ci, t, X, eu); #c1(EIF3F) c2(NP_003745) c3(eukaryotic translation initiation factor 3 subunit F) c4(140569, 140570, 140571, 140572) c5(140573, 140574, 140575, 140576, 140577, 140578, 140579, 140580, 140581, 140582, 140583, 140584, 140585, 140586, 140587, 140588, 140589, 140590, 140591, 140592, 140593, 140594, 140595, 140596, 140597, 140598, 140599, 140600, 140601) c6( )c7(T, aC, aE, Y, Z, eJ); #c1(EIF3H) c2(NP_003747) c3(eukaryotic translation initiation factor 3 subunit H) c4(140602, 140603) c5(140604, 140605, 140606, 140607, 140608, 140609, 140610, 140611, 140612, 140613, 140614, 140615) c6( )c7(T, RT, eM, aW, aT, r, DO, ci, q, ae, B, ad, hX); #c1 (EIF3J) c2(NP_001271265) c3(eukaryotic translation initiation factor 3 subunit J isoform 3) c4( )c5(103779, 103780, 103781, 103782, 103783, 103784) c6( )c7(T, bz, eM); #c1(EIF3J) c2(NP_003749) c3(eukaryotic translation initiation factor 3 subunit J isoform 1) c4(140616) c5( )c6( )c7(T, bz, eM); #c1(EIF3K) c2(NP_037366) c3(eukaryotic translation initiation factor 3 subunit K isoform 1) c4(175044, 175045, 175046) c5(175047, 175048, 175049, 175050, 175051, 175052, 175053, 175054, 175055, 175056, 175057, 175058, 175059) c6( )c7(bL, bM, T, dD, Ap, lD, ea, cV, jh, aNG, j, fu, blp, eq, vY, UX, q, cd, bv, agd, no, cc, lg, WY, oj, Ak, kf, h, bl, K, bU, aDU, hm, ly, hM, acV, Dg, asp, lW, xz, xs, Bw, Ad, lb, uY, aaO, kC, dr, lx, HT, ck, dR, op, lJ, blo, xA, c, QG, vA, bln, km, gZ, aDx, oh, mf, aS, Fw, rx, acJ, Xu, sT, B, atO); #c1(EIF3M) c2(NP_006351) c3(eukaryotic translation initiation factor 3 subunit M) c4(164263) c5(164264, 164265, 164266, 164267, 164268, 164269) c6( )c7(bB, bE, T, j); #c1(EIF4AI) c2(NP_001191439) c3(eukaryotic initiation factor 4A-1 isoform 2) c4(81519) c5(81520, 81521, 81522, 81523, 81524, 81525, 81526, 81527, 81528, 81529, 81530, 81531, 81532, 81533, 81534, 81535, 81536, 81537) c6( ) c7(Ye, ck, T, fM, aW, ds, jx, jh, aT, h, K); #c1(EIF4A1) c2(NP_001407) c3(eukaryotic initiation factor 4A-1 isoform 1) c4(117650, 117651, 117652) c5(117653, 117654, 117655, 117656, 117657, 117658) c6( )c7(Ye, ck, T, fM, aW, ds, jx, jh, aT, h, K); #c1(EIF4A2) c2(NP_001958) c3(eukaryotic initiation factor 4A-Il) c4(122620, 122621, 122622, 122623, 122624) c5(122625, 122626, 122627, 122628, 122629, 122630, 122631, 122632, 122633, 122634, 122635, 122636, 122637, 122638, 122639, 122640, 122641, 122642, 122643, 122644, 122645, 122646, 122647, 122648, 122649, 122650, 122651, 122652, 122653, 122654) c6( )c7(bL, ck, T, fM, ib, eM, pf, aW, ds, jx, jh, aT, K, bz, eW); #c1(EIF4A3) c2(NP_055555) c3(eukaryotic initiation factor 4A-Ill) c4(181915) c5(181916, 181917, 181918, 181919, 181920, 181921) c6( )c7(aC, arB, Dr, aA, blq, aF, qN); #c1(EIF4B) c2(NP_001408) c3(eukaryotic translation initiation factor 48 isoform 2) c4(117659, 117660) c5(117661, 117662, 117663, 117664, 117665, 117666, 117667, 117668, 117669, 117670, 117671, 117672) c6( )c7(en, ae, ff, b0); #c1(EIF4E) c2(NP_001124150) c3(eukaryotic translation initiation factor 4E isoform 3) c4(48299) c5(48300, 48301, 48302, 48303, 48304, 48305) c6( )c7(T, fR, pf, if, fd, bB, ci, aE, il, hu, iE, aW, Ku, g, bw, eM, nB, AM, m, ht, NT, n, oY, nA, qN, aCi, eW, jz, eV, By, Dz, ask, pi, afu, avP, aq, kp, cM, hL, it, B, L, jx, ex, aA, blr, hy, Kh, C, AO, q, eA, aT, ae, s, Y, ev, r, lo, en, cl, t, ik, bN, ck, Qi, iM, kP, nM, kB, au, CZ, bt, id, jh, Gn, xg, hF, ad, av, ib, db, vJ, ds, d, je, ff, cr, eZ, aF, iR, bz, aU, RV, bE, X, KS, iu); #c1(EIF4E) c2(NP_001124151) c3(eukaryotic translation initiation factor 4E isoform 2) c4(48306) c5(48307, 48308, 48309, 48310, 48311, 48312) c6( )c7(T, FR, pf, if, fd, bB, ci, aE, il, hu, iE, aW, Ku, g, bw, eM, nB, AM, m, ht, NT, n, oY, nA, qN, aCi, eW, jz, eV, By, Dz, ask, pi, afu, avP, aq, kp, cM, hL, it, B, L, jx, eX, aA, blr, hy, Kh, C, AO, q, eA, aT, ae, s, Y, ev, r, lo, en, cl, t, ik, bN, ck, Qi, iM, kP, nM, kB, au, CZ, bt, id, jh, Gn, xg, hF, ad, av, ib, db, vJ, ds, d, je, ff, cr, eZ, aF, iR, bz, aU, RV, bE, X, kS, iu); #c1(EIF4E) c2(NP_001959) c3(eukaryotic translation initiation factor 4E isoform 1) c4(122655) c5(122656, 122657, 122658, 122659, 122660, 122661) c6( )c7(T, fR, pf, if, fd, bB, ci, aE, il, hu, iE, aW, Ku, g, bw, eM, nB, AM, m, ht, NT, n, oY, nA, qN, aCi, eW, jz, eV, By, Dz, ask, pi, afu, avP, aq, kp, cM, hL, it, B, L, jx, eX, aA, blr, hy, Kh, C, AO, q, eA, aT, ae, s, Y, ev, r, lo, en, cl, t, ik, bN, ck, Di, iM, kP, nM, KB, au, CZ, bt, id, jh, Gn, xg, hF, ad, av, ib, db, vJ, ds, d, je, ff, cr, eZ, aF, iR, bz, aU, RV, bE, X, KS, iu); #c1(EIF4E2) c2(NP_001263265) c3(eukaryotic translation initiation factor 4E type 2 isoform C) c4(97/10) c5(97111, 97112, 97113, 97114, 97115, 97116) c6( )c7(T, eM); #c1(EIF4E2) c2(NP_001263266) c3(eukaryotic translation initiation factor 4E type 2 isoform D) c4(97117) c5(97118, 97119, 97120, 97121, 97122, 97123) c6( )c7(T, eM); #c1(EIF4E2) c2(NP_004837) c3(eukaryotic translation initiation factor 4E type 2 isoform A) c4(150181) c5(150182, 150183, 150184, 150185, 150186, 150187) c6( )c7(T, eM); #c1(EIF4E3) c2(NP_001128121) c3(eukaryotic translation initiation factor 4E type 3 isoform b) c4( )c5(49557, 49558, 49559, 49560, 49561, 49562, 49563, 49564, 49565, 49566, 49567, 49568, 49569, 49570, 49571, 49572, 49573, 49574, 49575, 49576, 49577, 49578, 49579, 49580, 49581, 49582, 49583, 49584, 49585) c6( )c7(aW, q, r, aT); #c1(EIF4E3) c2(NP_001128123) c3(eukaryotic translation initiation factor 4E type 3 isoform a) c4(49586, 49587, 49588, 49589, 49590) c5(49591, 49592, 49593, 49594, 49595, 49596, 49597, 49598, 49599, 49600, 49601, 49602, 49603) c6( )c7(aW, q, r, aT); #c1(EIF4EBP1) c2(NP_004086) c3(eukaryotic translation initiation factor 4E-binding protein 1) c4(143453) c5(143454, 143455, 143456, 143457, 143458, 143459) c6( )c7(T, kh, fM, hl, iH, eR, jx, jh, fd, bB, GC, aj, dG, v, ci, C, so, aY, q, ad, J, eA, dP, iu, eW, jz, aE, aT, iv, jy, h, bK, bl, jj, ae, af, cp, li, Nr, d, s, il, aW, Y, oV, ev, eZ, r, po, aF, cl, bz, hq, Nq, es, eM, bE, bt, it, B); #c1(EIF4EBP2) c2(NP_004087) c3(eukaryotic translation initiation factor 4E-binding protein 2) c4(143460) c5(143461, 143462, 143463, 143464, 143465, 143466) c6( )c7(T, eZ, r, jy, aj, v, t, q, J, bN); #c1(EIF4EBP3) c2(NP_003723) c3(eukaryotic translation initiation factor 4E-binding protein 3) c4(140451) c5(140452, 140453, 140454, 140455, 140456, 140457) c6( )c7(YE, q, r); #c1(EIF4ENIF1) c2(NP_001157974) c3(eukaryotic translation initiation factor 4E transporter isoform b) c4(62973, 62974) c5(62975, 62976, 62977, 62978, 62979, 62980) c6( )c7(vJ, il, EQ); #c1(EIF4ENIF1) c2(XP_006724344) c3(eukaryotic translation initiation factor 4E transporter isoform X2) c4(295945, 295946) c5( )c6( )c7(vJ, il, EQ); #c1(EIF4ENIF1) c2(XP_011528582) c3(eukaryotic translation initiation factor 4E transporter isoform X1) c4(323759, 323760, 323761) c5(323762, 323763, 323764, 323765, 323766, 323767, 323768, 323769, 323770, 323771, 323772, 323773, 323774, 323775, 323776, 323777, 323778, 323779, 323780, 323781, 323782, 323783) c6( )c7(vJ, il, EQ); #c1(EIF4ENIF1) c2(XP_011528584) c3(eukaryotic translation initiation factor 4E transporter isoform X3) c4( )c5(323784, 323785, 323786, 323787, 323788, 323789, 323790, 323791, 323792, 323793, 323794, 323795, 323796, 323797, 323798, 323799, 323800, 323801, 323802, 323803, 323804, 323805) c6( ) c7(vJ, il, EQ); #c1(EIF4G1) c2(NP_001181876) c3(eukaryotic translation initiation factor 4 gamma 1 isoform 6) c4(74281) c5(74282, 74283, 74284, 74285, 74286, 74287, 74288, 74289, 74290, 74291, 74292, 74293, 74294, 74295, 74296, 74297, 74298, 74299, 74300, 74301, 74302, 74303, 74304, 74305, 74306, 74307) c6( )c7(bls, T, BD, fR, pf, jx, jh, aYc, ci, eW, blt, ib, ds, aT, h, K, an, afu, ak, MD, aW, ff, eZ, en, Qu, bdP); #c1(EIF4G1) c2(NP_001278086) c3(eukaryotic translation initiation factor 4 gamma 1 isoform 7) c4(109448, 109449, 109450, 109451) c5(109452, 109453, 109454, 109455, 109456, 109457, 109458, 109459, 109460, 109461, 109462, 109463, 109464, 109465, 109466, 109467, 109468, 109469, 109470, 109471, 109472, 109473, 109474, 109475, 109476, 109477, 109478, 109479, 109480, 109481) c6( )c7(bls, T, BD, fR, pf, jx, jh, aYc, ci, eW, blt, ib, ds, aT, h, K, an, afu, ak, MD, aW, ff, eZ, en, Qu, bdP); #c1(EIF4G1) c2(NP_004944) c3(eukaryotic translation initiation factor 4 gamma 1 isoform 4) c4(151364, 151365, 151366) c5(151367, 151368, 151369, 151370, 151371, 151372, 151373, 151374, 151375, 151376, 151377, 151378, 151379, 151380, 151381) c6( )c7(bls, T, BD, fR, pf, jx, jh, aYc, ci, eW, blt, ib, ds, aT, h, K, an, afu, ak, MD, aW, ff, eZ, en, Qu, bdP); #c1(EIF4G1) c2(NP_886553) c3(eukaryotic translation initiation factor 4 gamma 1 isoform 1) c4(257226, 257227, 257228, 257229, 257230) c5(257231, 257232, 257233, 257234, 257235, 257236, 257237, 257238, 257239, 257240, 257241, 257242, 257243, 257244, 257245, 257246, 257247, 257248, 257249, 257250, 257251, 257252, 257253, 257254, 257255) c6( )c7(bls, T, BD, fR, pf, jx, jh, aYc, ci, eW, blt, ib, ds, aT, h, K, an, afu, ak, MD, aW, ff, eZ, en, Qu, bdP); #c1(EIF 4G1) c2(NP_937884) c3(eukaryotic translation initiation factor 4 gamma 1 isoform 5) c4(259423, 259424) c5(259425, 259426, 259427, 259428, 259429, 259430, 259431, 259432, 259433, 259434, 259435, 259436, 259437, 259438) c6( )c7(bls, T, BD, fR, pf, jx, jh, aYc, ci, eW, blt, ib, ds, aT, h, K, an, afu, ak, MD, aW, ff, eZ, en, Qu, bdP); #c1(EIF4G1) c2(NP_937885) c3(eukaryotic translation initiation factor 4 gamma 1 isoform 3) c4(259439) c5(259440, 259441, 259442) c6( )c7(bls, T, BD, fR, pf, jx, jh, aYc, ci, eW, blt, ib, ds, aT, h, K, an, afu, ak, MD, aW, ff, eZ, en, Qu, bdP); #c1(EIF4G2) c2(NP_001036024) c3(eukaryotic translation initiation factor 4 gamma 2 isoform 2) c4(33707, 33708, 33709, 33710) c5(33711, 33712, 33713, 33714, 33715, 33716, 33717, 33718, 33719, 33720, 33721, 33722, 33723, 33724, 33725, 33726, 33727, 33728, 33729, 33730, 33731, 33732, 33733, 33734, 33735, 33736, 33737, 33738, 33739, 33740, 33741, 33742, 33743, 33744, 33745, 33746, 33747, 33748, 33749, 33750, 33751, 33752, 33753, 33754, 33755, 33756, 33757, 33758, 33759, 33760, 33761, 33762, 33763) c6( )c7(gx, azs, T, id, if, iW, aA, bB, ci, qN, q, ad, av, aE, amJ, oj, aT, alO, dL, K, Od, jj, ae, af, bkl, eL, avV, s, aW, Y, ff, r, af, en, cm, ik, bz, cT, apM, bb, c, Kv, bE, U, B, blu); #c1(EIF4G2) c2(NP_001166176) c3(eukaryotic translation initiation factor 4 gamma 2 isoform 1) c4(68441, 68442, 68443, 68444, 68445, 68446, 68447) c5(68448, 68449, 68450, 68451, 68452, 68453, 68454, 68455, 68456, 68457, 68458, 68459, 68460, 68461, 68462, 68463, 68464, 68465, 68466, 68467, 68468) c6( )c7(gx, azs, T, id, if, iW, aA, bB, ci, qN, q, ad, av, aE, amJ, oj, aT, alO, dL, K, Od, jj, ae, af, bkl, eL, avV, s, aW, Y, ff, r, aF, en, cm, ik, bz, cT, apM, bb, c, Kv, bE, U, B, blu); #c1(EIF4H) c2(NP_071496) c3(eukaryotic translation initiation factor 4H isoform 1) c4(211776) c5(211777, 211778, 211779, 211780, 211781, 211782, 211783) c6( )c7(bL, eW, T, A, alC, bE, cz, r, bB, j, y, q); #c1(EIF4H) c2(NP_114381) c3(eukaryotic translation initiation factor 4H isoform 2) c4(222524) c5(222525, 222526, 222527, 222528, 222529, 222530) c6( )c7(bL, eW, T, A, alC, bE, cz, r, bB, j, y, q); #c1(EIF5) c2(NP_892116) c3(eukaryotic translation initiation factor 5) c4(257701, 257702, 257703, 257704, 257705, 257706, 257707) c5(257708, 257709, 257710, 257711, 257712, 257713, 257714, 257715, 257716, 257717, 257718, 257719, 257720, 257721, 257722, 257723, 257724, 257725, 257726, 257727, 257728, 257729, 257730, 257731, 257732, 257733, 257734, 257735, 257736, 257737, 257738, 257739, 257740, 257741, 257742, 257743, 257744, 257745, 257746, 257747, 257748, 257749, 257750, 257751, 257752, 257753, 257754, 257755, 257756, 257757, 257758, 257759, 257760, 257761) c6( )c7(av, dV, m, g, t, iu); #c1(EIF5A) c2(NP_001137232) c3(eukaryotic translation initiation factor 5A-1 isoform A) c4(55076) c5(55077, 55078, 55079, 55080, 55081, 55082, 55083, 55084, 55085, 55086) c6( )c7(T, aC, jh, bB, n, aj, ci, zP, eh, q, J, ad, av, iu, b, jZ, dV, h, K, ae, A, zl, d, r, g, t, bz, ck, eM, bE, m, i, B); #c1(EIF5A) c2(XP_005256566) c3(eukaryotic translation initiation factor 5A-1 isoform X1) c4(274203) c5(274204, 274205, 274206, 274207, 274208, 274209, 274210, 274211, 274212, 274213, 274214) c6( )c7(T, aC, jh, bB, n, aj, ci, zP, eh, q, J, ad, av, iu, b, jZ, dV, h, K, ae, A, zl, d, r, g, t, bz, ck, eM, bE, m, i, B); #c1(EIF5A) c2(XP_011522010) c3(eukaryotic translation initiation factor 5A-1 isoform X2) c4(316702) c5(316703) c6( )c7(T, aC, jh, bB, n, aj, ci, zP, eh, q, J, ad, av, iu, b, jZ, dV, h, K, ae, A, zl, d, r, g, t, bz, ck, eM, bE, m, i, B); #c1(EIF5A) c2(XP_011522015) c3(eukaryotic translation initiation factor 5A-1 isoform X3) c4( )c5(316704, 316705, 316706, 316707, 316708) c6( )c7(T, aC, jh, bB, n, aj, ci, zP, eh, q, J, ad, av, iu, b, jZ, dV, h, K, ae, A, zQ, d, r, g, t, bz, ck, eM, bE, m, i, B); #c1(EIF5A2) c2(NP_065123) c3(eukaryotic translation initiation factor 5A-2) c4(204060) c5(204061, 204062, 204063, 204064, 204065, 204066) c6( )c7(T, gU, d, id, eM, Y, it, n, dL, ci, m, q, t, on, eL, B); #c1(EIF6) c2(NP_001254739) c3(eukaryotic translation initiation factor 6 isoform a) c4(94902, 94903) c5(94904, 94905, 94906, 94907, 94908, 94909, 94910, 94911, 94912, 94913, 94914, 94915, 94916, 94917, 94918, 94919) c6( )c7(ck, T, s, aW, r, aT, aA, h, aF, ci, bz, q); #c1(EIF6) c2(NP_852131) c3(eukaryotic translation initiation factor 6 isoform c) c4(254901) c5(254902, 254903, 254904, 254905, 254906, 254907) c6( )c7(ck, T, s, aW, r, aT, aA, h, aF, ci, bz, q); #c1(ELAC1) c2(NP_061166) c3(zinc phosphodiesterase ELAC protein 1) c4(199693) c5(199694, 199695, 199696, 199697, 199698, 199699) c6( )c7(wR, ae, ad); #c1(ELAC2) c2(NP_001159434) c3(zinc phosphodiesterase ELAC protein 2 isoform 3) c4(64012, 64013) c5(64014, 64015, 64016, 64017, 64018, 64019, 64020, 64021, 64022, 64023, 64024, 64025) c6( )c7(bM, T, Ub, aM, blv, vc, blw, ci, ae, af, ad); #c1(ELAC2) c2(NP_060597) c3(zinc phosphodiesterase ELAC protein 2 isoform 1) c4(196740, 196741, 196742) c5(196743, 196744, 196745, 196746, 196747, 196748, 196749, 196750, 196751, 196752, 196753, 196754, 196755, 196756, 196757, 196758, 196759, 196760) c6( )c7(bM, T, Ub, aM, blv, vc, blw, ci, ae, af, ad); #c1(ELANE) c2(NP_001963) c3(neutrophil elastase preproprotein) c4(122673, 122674) c5(122675, 122676, 122677, 122678, 122679, 122680, 122681, 122682, 122683, 122684, 122685) c6( )c7(oO, bM, T, e, al, bB, fo, ci, wa, K, blx, dF, N, aa, aW, fc, bw, eM, iT, aWZ, uJ, pw, dD, aw, n, blz, cd, br, bly, ai, md, jy, eV, HX, wC, aXT, XE, aq, mo, iO, at, uz, B, fl, fG, oe, eX, rf, fF, afK, v, j, C, aT, cy, jj, af, il, f, ck, ac, hc, wR, fD, im, bt, zA, ao, kV, gV, am, df, Me, blA, bs, jh, blB, eq, av, ji, aXz, atJ, hG, eZ, aEW, aFB, bz, dr, bE, lF, cP, kS, hv); #c1(ELAVL1) c2(NP_001410) c3 (ELAV-like protein ( )c4(117673) c5(117674, 117675, 117676, 117677, 117678) c6( )c7(T, dD, aoT, aA, bB, eq, ci, eA, ip, dP, ib, aT, ab, K, jf, d, s, aW, Y, ff, eZ, CQ, en, g, ik, DT, c, bE, AH, aEs, m, y, X); #c1 (ELAVL1) c2(XP_011526079) c3(ELAV-like protein 1 isoform X1) c4(320386) c5(320387, 320388, 320389, 320390, 320391, 320392, 320393) c6( )c7(T, dD, aoT, aA, bB, eq, ci, eA, ip, dP, ib, aT, ab, K, jf, d, s, aW, Y, ff, eZ, CO, en, g, ik, DT, c, bE, AH, aEs, m, y, X); #c1(ELAVL2) c2(NP_001164668) c3(ELAV-like protein 2 isoform b) c4( )c5(67149, 67150, 67151, 67152, 67153, 67154, 67155, 67156, 67157, 67158, 67159, 67160, 67161, 67162, 67163, 67164, 67165, 67166, 67167) c6( )c7(bL, bM, T, bev, oe, EQ, aA, bB, Hn, j, pM, cd, ad, eA, av, ft, pN, aT, bK, he, af, aW, Y, aF, cl, cm, bz, ac, c, eM, bE, bt); #c1(ELAVL2) c2(NP_004423) c3 (ELAV-like protein 2 isoform a) c4( )c5(146249, 146250, 146251, 146252, 146253, 146254, 146255, 146256, 146257, 146258, 146259) c6( )c7(bL, bM, T, bev, oe, EQ, aA, bB, Hn, j, pM, cd, ad, eA, av, ft, pN, aT, bK, he, af, aW, Y, aF, cl, cm, bz, ac, c, eM, bE, bt); #c1(ELAVL2) c2(XP_011516076) c3(ELAV-like protein 2 isoform X1) c4(309280) c5(309281, 309282, 309283) c6( )c7(bL, bM, T, bev, oe, EQ, aA, bB, Hn, j, pM, cd, ad, eA, av, ft, pN, aT, bK, he, af, aW, Y, aF, cl, cm, bz, ac, c, eM, bE, bt); #c1(ELAVL2) c2(XP_011516077) c3(ELAV-like protein 2 isoform X2) c4(309284) c5(309285, 309286, 309287, 309288, 309289, 309290) c6( )c7(bL, bM, T, bev, oe, EQ, aA, bB, Hn, j, pM, cd, ad, eA, av, ft, pN, aT, bK, he, af, aW, Y, aF, cl, cm, bz, ac, c, eM, bE, bt); #c1(ELAVL2) c2(XP_011516079) c3(ELAV-like protein 2 isoform X4) c4(309291, 309292) c5(309293, 309294) c6( )c7(bL, bM, T, bev, oe, EQ, aA, bB, Hn, j, pM, cd, ad, eA, av, ft, pN, aT, bK, he, af, aW, Y, aF, cl, cm, bz, ac, c, eM, bE, bt); #c1(ELAVL2) c2(XP_011516083) c3(ELAV-like protein 2 isoform X6) c4(309295) c5( )c6( )c7(bL, bM, T, bev, De, EQ, aA, bB, Hn, j, pM, cd, ad, eA, av, ft, pN, aT, bK, he, af, aW, Y, aF, cl, cm, bz, ac, c, eM, bE, bt); #c1(ELAVL2) c2(XP_011516085) c3(ELAV-like protein 2 isoform X7) c4( )c5(309296) c6( )c7(bL, bM, T, bev, De, EQ, aA, bB, Hn, j, pM, cd, ad, eA, av, ft, pN, aT, bK, he, af, aW, Y, aF, cl, cm, bz, ac, c, eM, bE, bt); #c1(ELAVL3) c2(NP_001411) c3(ELAV-like protein 3 isoform 1) c4(117679) c5(117680, 117681, 117682, (17683, 117684, 117685) c6( )c7(eW, kl, id, ft, akJ, aq, dL, eL, B); #c1(ELAVL3) c2(NP_115657) c3(ELAV-like protein 3 isoform 2) c4(223477) c5(223478, 223479, 223480, 223481, 223482, 223483) c6( )c7(eW, kl, id, ft, akJ, aq, dl, eL, B); #c1(ELAVL4) c2(NP_001281277) c3(ELAV-like protein 4 isoform 6) c4(11/712) c5(11/713, 111714, 111715, 111716, 111717, 111718) c6( )c7(av, bev, ft, My, cm, an, gT, bz, xt, cB); #c1(ELAVL4) c2(XP_011539193) c3(ELAV-like protein 4 isoform X5) c4(337326) c5(337327, 337328, 337329, 337330, 337331, 337332) c6( )c7(av, bev, ft, My, cm, an, gT, bz, xt, cB); #c1(ELF1) c2(NP_001138825) c3(ETS-related transcription factor Elf-1 isoform b) c4(57904, 57905) c5(57906, 57907, 57908, 57909, 57910, 57911) c6( )c7(bP, if, eB, eZ, h, qN, cl, bt, B, bc); #c1(ELF1) c2(XP_011533252) c3(ETS-related transcription factor Elf-1 isoform X1) c4(329413) c5(329414, 329415, 329416, 329417, 329418, 329419, 329420, 329421, 329422, 329423, 329424, 329425) c6( )c7(bP, if, eB, eZ, h, qN, cl, bt, B, bc); #c1(ELF1) c2(XP_011533253) c3(ETS-related transcription factor Elf-1 isoform X2) c4(329426) c5(329427, 329428, 329429, 329430, 329431, 329432, 329433) c6( )c7(bP, if, eB, eZ, h, qN, cl, bt, B, bc); #c1(ELF1) c2(XP_011533254) c3(ETS-related transcription factor Elf-1 isoform X3) c4( )c5(329434) c6( )c7(bP, if, eB, eZ, h, qN, cl, bt, B, bc); #c1 (ELF2) c2(NP_001263386) c3(ETS-related transcription factor Elf-2 isoform 3) c4(97227) c5(97228, 97229, 97230, 97231, 97232, 97233) c6( )c7(bt, vi, kH); #c1(ELF2) c2(NP_006865) c3(ETS-related transcription factor Elf-2 isoform 2) c4(168716) c5(168717, 168718, 168719, 168720, 168721, 168722, 168723) c6( )c7(bt, vi, kH); #c1(ELF2) c2(NP_973728) c3(ETS-related transcription factor Elf-2 isoform 1) c4(262683) c5(262684, 262685, 262686, 262687, 262688, 262689, 262690, 262691, 262692, 262693) c6( ) c7(bt, vi, kH); #c1(ELF2) c2(XP_005262860) c3(ETS-related transcription factor Elf-2 isoform X2) c4(278152) c5(278153, 278154, 278155, 278156, 278157, 278158) c6( )c7(bt, vi, kH); #c1(ELF2) c2(XP_005262862) c3(ETS-related transcription factor Elf-2 isoform X4) c4(278159) c5( )c6( )c7(bt, vi, kH); #c1(ELF2) c2(XP_005262863) c3(ETS-related transcription factor Elf-2 isoform X6) c4( ) c5(278160, 278161, 278162, 278163, 278164, 278165) c6( )c7(bt, vi, kH); #c1(ELF3) c2(NP_001107781) c3(ETS-related transcription factor Elf-3) c4(42977, 42978, 42979) c5(42980, 42981, 42982, 42983, 42984, 42985, 42986, 42987, 42988, 42989, 42990, 42991, 42992, 42993, 42994, 42995, 42996, 42997, 42998, 42999, 43000, 43001, 43002, 43003, 43004, 43005, 43006) c6( )c7(bM, T, aJJ, aYP, wu, rQ, Lx, bS, ct, rP, iJ, ad, py, aT, K, wm, dl, ae, aW, ff, aUG, en, g, Z, iK, bz, vl, blC, ag, dh, hO, Lq, y, bt, YK); #c1(ELF4) c2(XP_005262446) c3(ETS-related transcription factor Elf-4 isoform X1) c4(277910, 277911) c5(277912, 277913, 277914, 277915, 277916, 277917, 277918, 277919, 277920, 277921, 277922, 277923, 277924, 277925, 277926, 277927, 277928) c6( )c7(T, eG, d, aW, Rj, ih, aiM, aT, rC, fd, By, jh, m, anW, hh, t, bt, eA); #c1(ELF5) c2(NP_001230009) c3(ETS-related transcription factor Elf-5 isoform 3) c4(85780) c5(85781, 85782, 85783, 85784, 85785, 85786) c6( )c7(bM, VZ, c, hb, aW, aT, ci); #c1(ELF5) c2(NP_001413) c3(ETS-related transcription factor Elf-5 isoform 2) c4( )c5(117686, 117687, 117688, 117689, 117690) c6( )c7(bM, VZ, c, hb, aW, aT, ci); #c1(ELF5) c2(NP_938195) c3(ETS-related transcription factor Elf-5 isoform 1) c4(259703, 259704) c5(259705, 259706, 259707, 259708, 259709, 259710, 259711, 259712) c6( ) c7(bM, VZ, c, hb, aW, aT, ci); #c1(ELK1) c2(NP_001244097) c3(ETS domain-containing protein Elk-1 isoform b) c4(91916) c5(91917, 91918, 91919, 91920, 91921, 91922) c6( )c7(ID, dD, id, yS, ea, aA, eq, iJ, q, ad, mj, MR, nd, aqk, kf, aT, K, ae, ly, ak, eG, d, aW, eZ, r, nf, hN, vs, g, aal, cm, Ld, lx, U, yA, bt, B); #c1(ELK1) c2(NP_005220) c3(ETS domain-containing protein Elk-1 isoform a) c4(154165, 154166) c5(154167, 154168, 154169, 154170, 154171, 154172, 154173, 154174, 154175, 154/76, 154177, 154178, 154179, 154180, 154181, 154182, 154183, 154184, 154185, 154186) c6( )c7(ID, dD, id, yS, ea, aA, eq, iJ, q, ad, mj, MR, nd, aqk, kf, aT, K, ae, ly, ak, eG, d, aW, eZ, r, nf, hN, vs, g, aal, cm, Ld, lx, U, yA, bt, B); #c1(ELK3) c2(XP_006719338) c3(ETS domain-containing protein Elk-3 isoform X1) c4(292411, 292412, 292413) c5(292414, 292415, 292416, 292417, 292418, 292419, 292420, 292421, 292422, 292423, 292424, 292425, 292426, 292427, 292428, 292429, 292430, 292431, 292432) c6( )c7(T, aC, pY, RX, DV, bO, cz, oR, bS, ww, ct, C, aBl, Za, q, ft, agd, afm, sO, iX, pX, aT, Ps, bK, af, qE, aWm, blD, tb, aW, Zf, abi, Pg, r, hv, Z, aiO, aFA, ok, alC, ac, cA, eM, Ew, eB, VB, uz, wR, yH, aan, lw, Fl, agZ); #c1(ELK4) c2(NP_068567) c3(ETS domain-containing protein Elk-4 isoform b) c4(209418) c5(209419, 209420, 209421, 209422, 209423, 209424, 209425) c6( )c7(AK, pi, d, aW, Zf, bez, rS, dV, aT, r, wR, CZ, eA, C, Za, ae, q, ad); #c1(ELK4) c2(XP_005245008) c3(ETS domain-containing protein Elk-4 isoform X1) c4(266206, 266207) c5(266208, 266209, 266210, 266211, 266212, 266213, 266214, 266215, 266216, 266217, 266218, 266219) c6( ) c7(AK, pi, d, aW, Zf, bez, rS, dV, aT, r, wR, CZ, eA, C, Za, ae, q, ad); #c1(ELL) c2(NP_006523) c3(RNA polymerase II elongation factor ELL) c4(165978, 165979) c5(165980, 165981, 165982, 165983, 165984, 165985, 165986, 165987, 165988, 165989, 165990, 165991) c6( )c7(ck, T, iT, iZ, hF, bt, ae, ad); #c1(ELL) c2(XP_011526632) c3(RNA polymerase II elongation factor ELL isoform X1) c4( )c5(320934) c6( )c7(ck, T, iT, iZ, hF, bt, ae, ad); #c1(ELL2) c2(NP_036213) c3(RNA polymerase II elongation factor ELL2) c4(172403) c5(172404, 172405, 172406, 172407, 172408, 172409) c6( )c7(De); #c1(ELL2) c2(XP_011541582) c3(RNA polymerase II elongation factor ELL2 isoform X2) c4(340125) c5(340126, 340127, 340128, 340129, 340130, 340131) c6( )c7(ce); #c1(ELM01) c2(NP_569709) c3(engulfment and cell motility protein 1 isoform 2) c4( )c5(233731, 233732, 233733, 233734, 233735, 233736, 233737, 233738, 233739, 233740) c6( )c7(bL, iV, A, eR, pf, hb, aw, aT, dy, k, hp, eA, bw, fq, bc); #c1(ELM01) c2(XP_005249976) c3(engulfment and cell motility protein 1 isoform X1) c4(269961, 269962, 269963, 269964) c5(269965, 269966, 269967, 269968, 269969, 269970, 269971, 269972, 269973, 269974, 269975, 269976, 269977, 269978, 269979, 269980, 269981, 269982, 269983, 269984, 269985, 269986, 269987, 269988, 269989, 269990) c6( )c7(bL, iV, A, eR, pf, hb, aw, aT, dy, k, hp, eA, bw, fq, bc); #c1(ELM01) c2(XP_011513957) c3(engulfment and cell motility protein 1 isoform X2) c4(306384, 306385, 306386) c5(306387, 306388, 306389, 306390, 306391, 306392, 306393, 306394, 306395, 306396, 306397, 306398, 306399, 306400, 306401, 306402, 306403, 306404) c6( )c7(bL, iV, A, eR, pf, hb, aw, aT, dy, k, hp, eA, bw, fq, bc); #c1(ELM02) c2(NP_877496) c3(engulfment and cell motility protein 2) c4(256845, 256846, 256847, 256848, 256849) c5(256850, 256851, 256852, 256853, 256854, 256855, 256856, 256857, 256858, 256859, 256860, 256861, 256862, 256863, 256864, 256865, 256866, 256867, 256868, 256869, 256870, 256871, 256872, 256873, 256874, 256875, 256876, 256877, 256878) c6( )c7(bL); #c1(ELM02) c2(XP_005260559) c3(engulfment and cell motility protein 2 isoform X3) c4( )c5(276609, 276610, 276611, 276612, 276613, 276614, 276615, 276616, 276617, 276618, 276619, 276620, 276621, 276622, 276623, 276624, 276625, 276626) c6( ) c7(bl); #c1(ELMOD1) c2(NP_001123509) c3(ELMO domain-containing protein 1 isoform 2) c4(47260) c5(47261, 47262, 47263, 47264, 47265, 47266) c6( )c7(beA, cF, ac); #c1(ELMOD1) c2(NP_061182) c3(ELMO domain-containing protein 1 isoform 1) c4(199816) c5(199817, 199818, 199819, 199820, 199821, 199822) c6( )c7(beA, cF, ac); #c1(ELMOD2) c2(NP_714913) c3(ELMO domain-containing protein 2) c4(246602) c5(246603, 246604, 246605, 246606, 246607, 246608, 246609, 246610, 246611, 246612, 246613, 246614, 246615, 246616, 246617) c6( )c7(LB); #c1(ELMOD2) c2(XP_005262942) c3(ELMO domain-containing protein 2 isoform X2) c4(278173) c5(278174) c6( ) c7(LB); #c1(ELMOD2) c2(XP_011530120) c3(ELMO domain-containing protein 2 isoform X3) c4(326027) c5(326028, 326029) c6( ) c7(LB); #c1(ELMOD3) c2(NP_001128494) c3(ELMO domain-containing protein 3 isoform b) c4(50080, 50081) c5(50082, 50083, 50084, 50085, 50086, 50087, 50088, 50089, 50090, 50091, 50092, 50093, 50094, 50095, 50096, 50097, 50098, 50099) c6( )c7(Bh, blE); #c1(ELMOD3) c2(NP_115589) c3(ELMO domain-containing protein 3 isoform a) c4(223295, 223296, 223297) c5(223298, 223299, 223300, 223301, 223302, 223303) c6( )c7(Bh, blE); #c1(ELMOD3) c2(XP_006712179) c3(ELMO domain-containing protein 3 isoform X2) c4( )c5(286494, 286495, 286496, 286497, 286498, 286499, 286500, 286501, 286502, 286503, 286504, 286505, 286506, 286507, 286508, 286509, 286510, 286511) c6( )c7(Bh, blE); #c1(ELMSAN1) c2(NP_919254) c3(ELM2 and SANT domain-containing protein 1) c4( ) c5(258307, 258308, 258309, 258310, 258311, 258312, 258313, 258314, 258315, 258316, 258317, 258318, 258319, 258320, 258321, 258322, 258323, 258324, 258325) c6( )c7(gv); #c1(ELMSAN1) c2(XP_005268263) c3(ELM2 and SANT domain-containing protein 1 isoform X1) c4(281649, 281650, 281651) c5(281652) c6( )c7(gv); #c1(ELMSAN1) c2(XP_011535621) c3(ELM2 and SANT domain-containing protein 1 isoform X2) c4( )c5(332876, 332877, 332878, 332879, 332880, 332881) c6( )c7(gv); #c1(ELN) c2(NP_000492) c3(elastin isoform a precursor) c4(9915) c5( )c6( )c7(blK, T, lP, ea, e, al, Av, fo, sD, Hr, wa, x, bll, HR, K, pk, N, aa, li, bbM, aW, uC, lq, uo, uJ, vF, blL, Vj, dD, vl, FG, RX, aw, ww, blH, kv, nA, bd, cd, cc, azj, rC, ux, db, Um, wC, aiB, blF, aJ, blP, ail, dw, W, vD, bP, blM, wk, bm, at, uz, rx, bD, alo, fl, eR, fG, cz, lj, j, Eh, Mg, aCF, aT, blN, bq, af, blG, bf, oV, Y, blJ, aYR, gm, bQ, bN, alC, pZ, fD, afp, bt, sY, am, ha, eq, Pu, fU, hd, Ot, le, lQ, lz, mu, d, tb, cr, dr, qa, nb, Uj, Ny, blO, aS, Hg, be, GF, lC, ei, us); #c1(ELN) c2(NP_001075221) c3(elastin isoform b precursor) c4(37267, 37268) c5( )c6( )c7(blK, T, lP, ea, e, al, Av, fo, sD, Hr, wa, x, bll, HR, K, pk, N, aa, li, bbM, aW, uC, lq, uo, uJ, vF, blL, Vj, dD, vl, FG, RX, aw, ww, blH, kv, nA, bd, cd, cc, azj, rC, ux, db, Um, WC, aiB, blF, aJ, blP, ail, dw, W, vD, bP, blM, wk, bm, at, uz, rx, bD, alo, fl, eR, fG, cz, lj, j, Eh, Mg, aCF, aT, blN, bq, af, blG, bf, oV, Y, blJ, aYR, gm, bQ, bN, alC, pZ, fD, afp, bt, sY, am, ha, eq, Pu, fU, hd, Ot, le, lQ, lz, mu, d, tb, cr, dr, qa, nb, Uj, Ny, blO, aS, Hg, be, GF, lC, ei, us); #c1(ELN) c2(NP_001075222) c3(elastin isoform c precursor) c4(37269) c5( )c6( )c7(blK, T, lP, ea, e, al, Av, fo, sD, Hr, wa, x, bll, HR, K, pk, N, aa, li, bbM, aW, uC, lq, uo, uJ, vF, blL, Vj, dD, vl, FG, RX, aw, ww, blH, kv, nA, bd, cd, cc, azj, rC, ux, db, Um, WC, aiB, blF, aJ, blP, ail, dw, W, vD, bP, blM, wk, bm, at, uz, rx, bD, alo, fl, eR, fG, cz, lj, j, Eh, Mg, aCF, aT, blN, bq, af, blG, bf, oV, Y, blJ, aYR, gm, bQ, bN, alC, pZ, fD, afp, bt, sY, am, ha, eq, Pu, fU, hd, Ot, le, lQ, lz, mu, d, tb, cr, dr, qa, nb, Uj, Ny, blO, aS, Hg, be, GF, lC, ei, us); #c1(ELN) c2(NP_001075223) c3(elastin isoform d precursor) c4(37270) c5( )c6( )c7(blK, T, lP, ea, e, al, Av, fo, sD, Hr, wa, x, bll, HR, K, pk, N, aa, li, bbM, aW, uC, lq, uo, uJ, vF, blL, Vj, dD, vl, FG, RX, aw, ww, blH, kv, nA, bd, cd, cc, azj, rC, ux, db, Um, wC, aiB, blF, aJ, blP, ail, dw, W, vD, bP, blM, wk, bm, at, uz, rx, bD, alo, fl, eR, fG, cz, lj, j, Eh, Mg, aCF, aT, blN, bq, af, blG, bf, oV, Y, blJ, aYR, gm, bQ, bN, alC, pZ, fD, afp, bt, sY, am, ha, eq, Pu, fU, hd, Ot, le, lQ, lz, mu, d, tb, cr, dr, qa, nb, Uj, Ny, blO, aS, Hg, be, GF, lC, ei, us); #c1 (ELN) c2(NP_001075224) c3(elastin isoform e precursor) c4(37271) c5( )c6( )c7(blK, T, lP, ea, e, al, Av, fo, sD, Hr, wa, x, bll, HR, K, pk, N, aa, li, bbM, aW, uC, lq, uo, uJ, vF, blL, Vj, dD, vl, FG, RX, aw, ww, blH, kv, nA, bd, cd, cc, azj, rC, ux, db, Um, wC, aiB, blF, aJ, blP, ail, dw, W, vD, bP, blM, wk, bm, at, uz, rx, bD, alo, fl, eR, fG, cz, lj, j, Eh, Mg, aCF, aT, blN, bq, af, blG, bf, oV, Y, blJ, aYR, gm, bQ, bN, alC, pZ, fD, afp, bt, sY, am, ha, eq, Pu, fU, hd, lt, le, lQ, lz, mu, d, tb, cr, dr, qa, nb, Uj, Ny, blO, aS, Hg, be, GF, lC, ei, us); #c1(ELN) c2(NP_001265841) c3(elastin isoform f precursor) c4(99341) c5( )c6( )c7(blK, T, lP, ea, e, al, Av, fo, sD, Hr, wa, x, bll, HR, K, pk, N, aa, li, bbM, aW, uC, lq, uo, uJ, vF, blL, Vj, dD, vl, FG, RX, aw, ww, blH, kv, nA, bd, cd, cc, azj, rC, ux, db, Um, wC, aiB, blF, aJ, blP, ail, dw, W, vD, bP, blM, wk, bm, at, uz, rx, bD, alo, fl, eR, fG, cz, lj, j, Eh, Mg, aCF, aT, blN, bq, af, blG, bf, oV, Y, blJ, aYR, gm, bQ, bN, alC, pZ, fD, afp, bt, sY, am, ha, eq, Pu, fU, hd, Ot, le, lQ, lz, mu, d, tb, cr, dr, qa, nb, Uj, Ny, blO, aS, Hg, be, GF, lC, ei, us); #c1 (ELN) c2(NP_001265846) c3 (elastin isoform k precursor) c4(99342) c5( )c6( )c7(blK, T, lP, ea, e, al, Av, fo, sD, Hr, wa, x, bll, HR, K, pk, N, aa, li, bbM, aW, uC, lq, uo, uJ, vF, blL, Vj, dD, vl, FG, RX, aw, ww, blH, kv, nA, bd, cd, cc, azj, rC, ux, db, Um, wC, aiB, blF, aJ, blP, ail, dw, W, vD, bP, blM, wk, bm, at, uz, rx, bD, alo, fl, eR, fG, cz, lj, j, Eh, Mg, aCF, aT, blN, bq, af, blG, bf, oV, Y, blJ, aYR, gm, bQ, bN, alC, pZ, fD, afp, bt, sY, am, ha, eq, Pu, fU, hd, Ot, le, lQ, lz, mu, d, tb, cr, dr, qa, nb, Uj, Ny, blO, aS, Hg, be, GF, lC, ei, us); #c1(ELN) c2(NP_001265847) c3(elastin isoform 1 precursor) c4(99343, 99344, 99345) c5(99346, 99347, 99348, 99349, 99350, 99351, 99352, 99353, 99354, 99355, 99356, 99357, 99358, 99359, 99360, 99361, 99362, 99363, 99364, 99365, 99366, 99367, 99368, 99369, 99370, 99371, 99372, 99373, 99374, 99375, 99376, 99377, 99378, 99379, 99380, 99381, 99382, 99383, 99384, 99385, 99386, 99387, 99388, 99389, 99390, 99391, 99392, 99393, 99394, 99395, 99396, 99397, 99398, 99399, 99400, 99401, 99402, 99403, 99404, 99405, 99406, 99407, 99408, 99409, 99410, 99411, 99412, 99413, 99414, 99415, 99416, 99417, 99418, 99419, 99420, 99421, 99422, 99423, 99424, 99425, 99426, 99427, 99428, 99429, 99430, 99431, 99432, 99433, 99434, 99435, 99436, 99437, 99438, 99439, 99440, 99441, 99442, 99443, 99444, 99445, 99446, 99447, 99448, 99449, 99450, 99451, 99452, 99453, 99454) c6(99455, 99456, 99457, 99458, 99459, 99460, 99461, 99462, 99463, 99464, 99465, 99466, 99467, 99468, 99469, 99470, 99471, 99472, 99473, 99474, 99475, 99476, 99477, 99478, 99479, 99480, 99481, 99482, 99483) c7(blK, T, lP, ea, e, al, Av, fo, sD, Hr, wa, x, bll, HR, K, pk, N, aa, li, bbM, aW, uC, lq, uo, uJ, vF, blL, Vj, dD, vl, FG, RX, aw, ww, blH, kv, nA, bd, cd, cc, azj, rC, ux, db, Um, wC, aiB, bIF, aJ, blP, ail, dw, W, vD, bP, blM, wk, bm, at, uz, rx, bD, alo, fl, eR, fG, cz, lj, j, Eh, Mg, aCF, aT, blN, bq, af, blG, bf, oV, Y, blJ, aYR, gm, bQ, bN, alC, pZ, fD, afp, bt, sY, am, ha, eq, Pu, fU, hd, Ot, le, lQ, lz, mu, d, tb, cr, dr, qa, nb, Uj, Ny, blO, aS, Hg, be, GF, lC, ei, us); #c1(ELN) c2(NP_001265868) c3(elastin isoform m precursor) c4(99545) c5(99546, 99547, 99548, 99549, 99550, 99551, 99552, 99553) c6( ) c7(blK, T, lP, ea, e, al, Av, fo, sD, Hr, wa, x, bll, HR, K, pk, N, aa, li, bbM, aW, uC, lq, uo, uJ, vF, blL, Vj, dD, vl, FG, RX, aw, ww, blH, kv, nA, bd, cd, cc, azj, rC, ux, db, Um, wC, aiB, blF, aJ, blP, ail, dw, W, vD, bP, blM, wk, bm, at, uz, rx, bD, alo, fl, eR, fG, cz, lj, j, Eh, Mg, aCF, aT, blN, bq, af, blG, bf, oV, Y, blJ, aYR, gm, bQ, bN, alC, pZ, fD, afp, bt, sY, am, ha, eq, Pu, fU, hd, Ot, le, lQ, lz, mu, d, tb, cr, dr, qa, nb, Uj, Ny, blO, aS, Hg, be, GF, lC, ei, us); #c1 (ELN) c2(XP_011514170) c3(elastin isoform X1) c4( )c5(306789, 306790) c6(306791) c7(blK, T, lP, ea, e, al, Av, fo, sD, Hr, wa, x, bll, HR, K, pk, N, aa, li, bbM, aW, uC, lq, uo, uJ, vF, blL, Vj, dD, vl, FG, RX, aw, ww, blH, kv, nA, bd, cd, cc, azj, rC, ux, db, Um, WC, aiB, blF, aJ, blP, ail, dw, W, vD, bP, blM, wk, bm, at, uz, rx, bD, alo, fl, eR, fG, cz, lj, j, Eh, Mg, aCF, aT, blN, bq, af, blG, bf, oV, Y, blJ, aYR, gm, bQ, bN, alC, pZ, fD, afp, bt, sY, am, ha, eq, Pu, fU, hd, Ot, le, lQ, lz, mu, d, tb, cr, dr, qa, nb, Uj, Ny, blO, aS, Hg, be, GF, lC, ei, us); #c1(ELN) c2(XP_011514176) c3(elastin isoform X9) c4(306792) c5( )c6( )c7(blK, T, lP, ea, e, al, Av, fo, sD, Hr, wa, x, bll, HR, K, pk, N, aa, li, bbM, aW, uC, lq, uo, uJ, vF, blL, Vj, dD, vl, FG, RX, aw, ww, blH, kv, nA, bd, cd, cc, azj, rC, ux, db, Um, wC, aiB, blF, aJ, blP, ail, dw, W, vD, bP, blM, wk, bm, at, uz, rx, bD, alo, fl, eR, fG, cz, lj, j, Eh, Mg, aCF, aT, blN, bq, af, blG, bf, oV, Y, blJ, aYR, gm, bQ, bN, alC, pZ, fD, afp, bt, sY, am, ha, eq, Pu, fU, hd, Ot, le, lQ, lz, mu, d, tb, cr, dr, qa, nb, Uj, Ny, blO, aS, Hg, be, GF, lC, ei, us); #c1(ELN) c2(XP_011514177) c3 (elastin isoform X10) c4(306793, 306794) c5( )c6( )c7(blK, T, lP, ea, e, al, Av, fo, sD, Hr, wa, x, bll, HR, K, pk, N, aa, li, bbM, aW, uC, lq, uo, uJ, vF, blL, Vj, dD, vl, FG, RX, aw, ww, blH, kv, nA, bd, cd, cc, azj, rC, ux, db, Um, wC, aiB, blF, aJ, blP, ail, dw, W, vD, bP, blM, wk, bm, at, uz, rx, bD, alo, fl, eR, fG, cz, lj, j, Eh, Mg, aCF, aT, blN, bq, af, blG, bf, oV, Y, blJ, aYR, gm, bQ, bN, alC, pZ, fD, afp, bt, sY, am, ha, eq, Pu, fU, hd, Ot, le, lQ, lz, mu, d, tb, cr, dr, qa, nb, Uj, Ny, blO, aS, Hg, be, GF, lC, ei, us); #c1(ELN) c2(XP_011514179) c3 (elastin isoform X12) c4( )c5(306795) c6( )c7(blK, T, lP, ea, e, al, Av, fo, sD, Hr, wa, x, bll, HR, K, pk, N, aa, li, bbM, aW, uC, lq, uo, uJ, vF, blL, Vj, dD, vl, FG, RX, aw, ww, blH, kv, nA, bd, cd, cc, azj, rC, ux, db, Um, wC, aiB, blF, aJ, blP, ail, dw, W, vD, bP, blM, wk, bm, at, uz, rx, bD, alo, fl, eR, fG, cz, lj, j, Eh, Mg, aCF, aT, blN, bq, af, blG, bf, oV, Y, blJ, aYR, gm, bQ, bN, alC, pZ, fD, afp, bt, sY, am, ha, eq, Pu, fU, hd, Ot, le, lQ, lz, mu, d, tb, cr, dr, qa, nb, Uj, Ny, blO, aS, Hg, be, GF, lC, ei, us); #c1(ELOF1) c2(NP_115753) c3(transcription elongation factor 1 homolog) c4(223831) c5(223832, 223833, 223834, 223835, 223836, 223837) c6( )c7(bP, if, eB, bt, eZ, h, qN, cl, V); #c1 (ELOVL2) c2(NP_060240) c3(elongation of very long chain fatty acids protein 2) c4( )c5(194925, 194926, 194927, 194928, 194929) c6( )c7(gU); #c1(ELOVL2) c2(XP_011513018) c3(elongation of very long chain fatty acids protein 2 isoform X1) c4( )c5(304627) c6( )c7(gU); #c1(ELOVL2) c2(XP_01513019) c3(elongation of very long chain fatty acids protein 2 isoform X2) c4(304628) c5( )c6( )c7(gU); #c1(ELOVL4) c2(NP_073563) c3(elongation of very long chain fatty acids protein 4) c4(212860) c5(212861, 212862, 212863, 212864, 212865, 212866) c6( )c7(li, T, cc, no, lR, nb, kB, W, nX, blS, Sr, lr, blR, nw, blQ, nC, Um, de); #c1(ELOVL5) c2(NP_001229757) c3(elongation of very long chain fatty acids protein 5 isoform 2) c4(85153) c5(85154, 85155, 85156, 85157, 85158, 85159) c6( )c7(HQ, dH, kB, rf, dG, v, dw); #c1(ELOVL5) c2(NP_001229760) c3(elongation of very long chain fatty acids protein 5 isoform 4) c4(85170) c5(85171, 85172, 85173, 85174, 85175, 85176) c6( )c7(HQ, dH, kB, rf, dG, v, dw); #c1(ELOVL5) c2(NP_001288785) c3(elongation of very long chain fatty acids protein 5 isoform 1) c4(114026, 114027) c5(114028, 114029, 114030, 114031, 114032, 114033, 114034, 114035, 114036, 114037, 114038, 114039, 114040, 114041, 114042, 114043) c6( )c7(HQ, dH, kB, rf, dG, v, dw); #c1(ELOVL6) c2(XP_011530536) c3(elongation of very long chain fatty acids protein 6 isoform X1) c4(326450, 326451, 326452) c5(326453, 326454, 326455, 326456, 326457, 326458, 326459, 326460, 326461, 326462, 326463, 326464, 326465, 326466, 326467, 326468, 326469, 326470, 326471, 326472, 326473, 326474) c6( )c7(LB, Kc, os, dD, wD, Jr, ea, fG, fJ, eE, cr, jy, oZ, v, eq, K, Xk, bX, ey, aAE, blT); #c1 (ELOVL7) c2(NP_001098028) c3(elongation of very long chain fatty acids protein 7 isoform 1) c4(41289, 41290) c5(41291, 41292, 41293, 41294, 41295, 41296, 41297, 41298, 41299, 41300, 41301, 41302, 41303, 41304, 41305, 41306, 41307, 41308, 41309, 41310, 41311, 41312, 41313, 41314) c6( )c7(IM, ae, ac, ad); #c1(ELP2) c2(NP_001229804) c3(elongator complex protein 2 isoform 1) c4(85258) c5(85259, 85260, 85261, 85262, 85263, 85264) c6( )c7(ao, m, K, nA); #c1(ELP2) c2(NP_001229805) c3(elongator complex protein 2 isoform 3) c4(85265) c5(85266, 85267, 85268, 85269, 85270, 85271) c6( )c7(ao, m, K, nA); #c1(ELP2) c2(NP_001229806) c3(elongator complex protein 2 isoform 4) c4(85272) c5(85273, 85274, 85275, 85276, 85277, 85278) c6( )c7(ao, m, K, nA); #c1(ELP2) c2(NP_001229807) c3(elongator complex protein 2 isoform 5) c4(85279) c5(85280, 85281, 85282, 85283, 85284, 85285) c6( )c7(ao, m, K, nA); #c1(ELP2) c2(NP_001229808) c3(elongator complex protein 2 isoform 6) c4(85286) c5(85287, 85288, 85289, 85290, 85291, 85292) c6( )c7(ao, m, K, nA); #c1(ELP2) c2(NP_060725) c3(elongator complex protein 2 isoform 2) c4(197475) c5(197476, 197477, 197478, 197479, 197480, 197481) c6( )c7(ao, m, K, nA); #c1(ELP3) c2(NP_001271151) c3(elongator complex protein 3 isoform 3) c4(103473, 103474) c5(103475, 103476, 103477, 103478, 103479, 103480, 103481, 103482, 103483, 103484, 103485, 103486, 103487, 103488, 103489, 103490) c6( )c7(aqJ, U, bM, c); #c1(ELP3) c2(NP_001271155) c3(elongator complex protein 3 isoform 5) c4( ) c5(103491, 103492, 103493, 103494, 103495) c6( )c7(aqJ, U, bM, c); #c1(ELP3) c2(NP_060561) c3(elongator complex protein 3 isoform 1) c4(196553, 196554, 196555) c5(196556, 196557, 196558, 196559, 196560, 196561, 196562, 196563, 196564) c6( )c7(aqJ, U, bM, c); #c1(ELP3) c2(XP_006716417) c3(elongator complex protein 3 isoform X1) c4(290287, 290288) c5(290289, 290290, 290291, 290292, 290293, 290294, 290295, 290296, 290297, 290298, 290299, 290300) c6( )c7(aqJ, U, bM, c); #c1(ELP4) c2(NP_001275654) c3(elongator complex protein 4 isoform 2) c4(106913) c5(106914, 106915, 106916, 106917, 106918, 106919) c6( )c7(akf, ko, lr); #c1(ELP4) c2(NP_001275655) c3(elongator complex protein 4 isoform 3) c4(106920, 106921) c5(106922, 106923, 106924, 106925, 106926, 106927, 106928, 106929, 106930, 106931, 106932, 106933, 106934, 106935, 106936, 106937, 106938, 106939, 106940, 106941, 106942, 106943, 106944, 106945, 106946, 106947, 106948, 106949, 106950, 106951, 106952) c6( )c7(akf, ko, lr); #c1(ELP4) c2(NP_061913) c3(elongator complex protein 4 isoform 1) c4(201168) c5(201169, 201170, 201171, 201172, 201173, 201174) c6( )c7(akf, ko, lr); #c1(ELPG) c2(NP_001026873) c3(elongator complex protein 6) c4(29764, 29765) c5(29766, 29767, 29768, 29769, 29770, 29771, 29772, 29773, 29774, 29775, 29776, 29777) c6( )c7(A, fu); #c1(ELSPBP1) c2(NP_071425) c3(epididymal sperm-binding protein 1 precursor) c4(211597) c5(211598, 211599, 211600, 211601, 211602, 211603, 211604, 211605) c6(211606, 211607) c7(fq, kh); #c1(EMB) c2(NP_940851) c3(embigin precursor) c4(259946) c5(259947, 259948, 259949, 259950, 259951, 259952) c6( )c7(ck, B, hc, fJ, jx, eD, eZ, h, wR, FL, bD, hE, mC, vi); #c1(EMC10) c2(NP_778233) c3(ER membrane protein complex subunit 10 isoform 1 precursor) c4(251473) c5(251474, 251475, 251476, 251477, 251478, 251479, 251480, 251481) c6(251482, 251483) c7(Fm, T, VJ, eA, as); #c1(EMC10) c2(NP_996261) c3(ER membrane protein complex subunit 10 isoform 2 precursor) c4(263977, 263978) c5(263979, 263980, 263981, 263982, 263983, 263984, 263985, 263986, 263987, 263988, 263989, 263990, 263991, 263992, 263993, 263994) c6(263995, 263996, 263997, 263998) c7(Fm, T, VJ, eA, as); #c1(EMC2) c2(NP_055488) c3(ER membrane protein complex subunit 2) c4(181471) c5(181472, 181473, 181474, 181475, 181476, 181477) c6( )c7(cd); #c1(EMC3) c2(NP_060917) c3(ER membrane protein complex subunit 3) c4(198602, 198603) c5(198604, 198605, 198606, 198607, 198608, 198609, 198610, 198611, 198612, 198613, 198614, 198615) c6( )c7(AW, mx, blU, vc, kp, bt); #c1(EMC7) c2(NP_064539) c3(ER membrane protein complex subunit 7 precursor) c4(202892) c5(202893, 202894, 202895, 202896, 202897, 202898) c6( )c7(cc); #c1(EMC8) c2(NP_001135760) c3(ER membrane protein complex subunit 8 isoform 2) c4(53131) c5(53132, 53133, 53134, 53135, 53136) c6( )c7(dy, ac, hy, ne, BN); #c1(EMC8) c2(NP_006058) c3(ER membrane protein complex subunit 8 isoform 1) c4(161521) c5(161522, 161523, 161524, 161525, 161526, 161527, 161528) c6( )c7(dy, ac, hy, ne, BN); #c1(EMCN) c2(NP_001153166) c3(endomucin isoform 2 precursor) c4(60471) c5(60472, 60473, 60474, 60475, 60476, 60477, 60478, 60479, 60480, 60481, 60482, 60483, 60484, 60485, 60486, 60487) c6(60488, 60489, 60490, 60491) c7(Jm, x, Vy, k, lQ, eE, y); #c1(EMCN) c2(XP_011530326) c3(endomucin isoform X1) c4(326167, 326168) c5(326169, 326170, 326171, 326172, 326173, 326174, 326175, 326176) c6(326177, 326178) c7(Jm, x, Vy, k, lQ, eE, y); #c1(EMD) c2(NP_000108) c3 (emerin) c4(1830, 1831) c5(1832, 1833, 1834, 1835, 1836, 1837, 1838, 1839, 1840, 1841, 1842, 1843, 1844) c6( )c7(dA, fR, mr, wL, aeu, aTl, bd, eW, er, cc, als, no, MR, QJ, oj, Ak, h, aWs, bl, hm, blV, aJ, xs, Ib, fe, uY, Bu, GR, Ye, ck, ly, iT, dB, mx, km, gZ, rx, Fw, y); #c1(EME1) c2(NP_689676) c3(crossover junction endonuclease EME1 isoform 2) c4(243533) c5(243534, 243535, 243536, 243537, 243538, 243539) c6( )c7(T, d, dB, bE, aT, bB, V, eA); #c1(EME1) c2(XP_011522698) c3(crossover junction endonuclease EME1 isoform X1) c4(317224) c5(317225, 317226, 317227, 317228, 317229, 317230, 317231, 317232, 317233, 317234) c6( )c7(T, d, dB, bE, aT, bB, V, eA); #c1(EMG1) c2(NP_006322) c3(ribosomal RNA small subunit methyltransferase NEP1) c4(164026) c5(164027, 164028, 164029, 164030, 164031, 164032) c6( )c7(h, blW, er, dx, aT); #c1(EMILIN1) c2(NP_008977) c3(EMILIN-1 precursor) c4(169994, 169995) c5(169996, 169997, 169998, 169999, 170000, 170001, 170002, 170003, 170004, 170005, 170006, 170007) c6( )c7(Vv, t, m, qa, cz); #c1(EMILIN2) c2(NP_114437) c3(EMILIN-2 precursor) c4(222774) c5(222775, 222776, 222777, 222778, 222779, 222780, 222781, 222782, 222783, 222784, 222785, 222786, 222787, 222788, 222789, 222790) c6(222791, 222792, 222793, 222794) c7(T, aC, aW, cz, aT, g); #c1(EMILIN2) c2(XP_011524049) c3(EMILIN-2 isoform X1) c4(318642) c5( )c6( )c7(T, aC, aW, cz, aT, g); #c1(EMILIN3) c2(NP_443078) c3(EMILIN-3 precursor) c4(229291) c5(229292, 229293, 229294, 229295, 229296, 229297, 229298, 229299) c6(229300, 229301) c7(ht); #c1(EML1) c2(NP_001008707) c3(echinoderm microtubule-associated protein-like 1 isoform a) c4(23370) c5(23371, 23372, 23373, 23374, 23375, 23376, 23377, 23378, 23379, 23380) c6( )c7(kp, af); #c1(EML1) c2(NP_004425) c3(echinoderm microtubule-associated protein-like 1 isoform b) c4(146260) c5(146261, 146262, 146263, 146264, 146265, 146266) c6( ) c7(kp, af); #c1(EML2) c2(NP_001180197) c3(echinoderm microtubule-associated protein-like 2 isoform 1) c4(73349, 73350) c5(73351, 73352, 73353, 73354, 73355, 73356, 73357, 73358, 73359, 73360, 73361, 73362) c6( )c7(aE, aW, Y, aT, uJ, ci); #c1(EML2) c2(NP_001180198) c3(echinoderm microtubule-associated protein-like 2 isoform 3) c4(73363) c5(73364, 73365, 73366, 73367, 73368, 73369) c6( )c7(aE, aW, Y, aT, uJ, ci); #c1(EML2) c2(NP_036287) c3(echinoderm microtubule-associated protein-like 2 isoform 2) c4(172847) c5(172848, 172849, 172850, 172851, 172852, 172853) c6( )c7(aE, aW, Y, aT, uJ, ci); #c1(EML2) c2(XP_011524999) c3(echinoderm microtubule-associated protein-like 2 isoform X6) c4(319476) c5(319477, 319478, 319479, 319480, 319481, 319482) c6( ) c7(aE, aW, Y, aT, uJ, ci); #c1(EML2) c2(XP_011525001) c3(echinoderm microtubule-associated protein-like 2 isoform X8) c4( ) c5(319483, 319484) c6( )c7(aE, aW, Y, aT, uJ, ci); #c1(EML4) c2(NP_001138548) c3(echinoderm microtubule-associated protein-like 4 isoform b) c4(56983) c5(56984, 56985, 56986, 56987, 56988, 56989) c6( )c7(YL, av, T, ft, Qy, eM, dB, ff, jh, n, ci, m, en, g, Tj, bz, iu); #c1(EML4) c2(NP_061936) c3(echinoderm microtubule-associated protein-like 4 isoform a) c4(201216) c5(201217, 201218, 201219, 201220, 201221, 201222) c6( )c7(YL, av, T, ft, Qy, eM, dB, ff, jh, n, ci, m, en, g, Tj, bz, iu); #c1(EML4) c2(XP_006712054) c3(echinoderm microtubule-associated protein-like 4 isoform X2) c4(286366) c5( )c6( )c7(YL, av, T, ft, Qy, eM, dB, ff, jh, n, ci, m, en, g, Tj, bz, iu); #c1(EML4) c2(XP_006712055) c3(echinoderm microtubule-associated protein-like 4 isoform X3) c4( )c5(286367, 286368, 286369, 286370, 286371, 286372) c6( )c7(YL, av, T, ft, Qy, eM, dB, ff, jh, n, ci, m, en, g, Tj, bz, iu); #c1(EML5) c2(NP_899243) c3(echinoderm microtubule-associated protein-like 5) c4(258068) c5(258069, 258070, 258071, 258072, 258073, 258074) c6( )c7(aE, aW, Y, aT, uJ, ci); #c1(EML5) c2(XP_006720133) c3(echinoderm microtubule-associated protein-like 5 isoform X6) c4(293270, 293271) c5(293272, 293273, 293274, 293275, 293276, 293277, 293278, 293279, 293280, 293281, 293282, 293283) c6( )c7(aE, aW, Y, aT, uJ, ci); #c1(EML6) c2(NP_001034842) c3(echinoderm microtubule-associated protein-like 6) c4(32357) c5(32358, 32359, 32360, 32361, 32362, 32363) c6( )c7(aC, v, pN); #c1(EMP1) c2(NP_001414) c3(epithelial membrane protein 1) c4(117691, 117692) c5(117693, 117694, 117695, 117696, 117697, 117698, 117699, 117700, 117701, 117702, 117703, 117704) c6( )c7(T, aw, aA, BF, yU, ad, Uh, aT, h, yZ, aDZ, ae, avV, aW, ff, aF, en, bz, ek, eM, dB, dO, eB, wR, EY); #c1(EMP2) c2(XP_006720927) c3(epithelial membrane protein 2 isoform X1) c4(293780) c5(293781, 293782, 293783, 293784, 293785, 293786, 293787, 293788, 293789, 293790) c6( )c7(T, id, hL, RC, ah, bU, cm); #c1(EMX1) c2(NP_004088) c3(homeobox protein EMX1) c4(143467) c5(143468, 143469, 143470, 143471, 143472, 143473, 143474) c6( )c7(yn); #c1(EMX2) c2(NP_001159396) c3(homeobox protein EMX2 isoform 2) c4(63948) c5(63949, 63950, 63951, 63952, 63953, 63954) c6( ) c7(T, aA, n, bS, j, dP, av, zY, wG, bK, Ur, iE, blX, aF, g, cl, Z, bz, uP, FB, hL, Gp, iu); #c1 (EMX2) c2(NP_004089) c3(homeobox protein EMX2 isoform 1) c4(143475) c5(143476, 143477, 143478, 143479, 143480, 143481, 143482) c6( )c7(T, aA, n, bS, j, dP, av, zY, wG, bK, Ur, iE, blX, aF, g, cl, Z, bz, uP, FB, hL, Gp, iu); #c1(ENI) c2(NP_001417) c3(homeobox protein engrailed-1) c4(117705) c5(117706, 117707, 117708, 117709, 117710, 117711, 117712, 117713, 117714) c6( )c7(V, qK, aR, arA, an); #c1(EN2) c2(NP_001418) c3(homeobox protein engrailed-2) c4(117715) c5(117716, 117717, 117718, 117719, 117720, 117721, 117722) c6( )c7(jf, Ng, T, iU, VT, aT, fr, an, rz, ae, ad, bN); #c1(ENAH) c2(NP_001008493) c3(protein enabled homolog isoform a) c4(23265) c5( )c6( )c7(eZ, aT, h, ey, q, ci, wt, cd); #c1(ENAH) c2(NP_060682) c3(protein enabled homolog isoform b) c4(197144) c5(197145, 197146, 197147, 197148, 197149, 197150) c6( )c7(eZ, aT, h, ey, q, ci, wt, cd); #c1(ENAH) c2(XP_011542536) c3(protein enabled homolog isoform X9) c4(341074) c5(341075, 341076, 341077, 341078, 341079, 341080) c6( ) c7(eZ, aT, h, ey, q, ci, wt, cd); #c1(ENAH) c2(XP_011542537) c3(protein enabled homolog isoform X10) c4( )c5(341081, 341082, 341083, 341084, 341085, 341086, 341087) c6( )c7(eZ, aT, h, ey, q, ci, wt, cd); #c1(ENAM) c2(NP_114095) c3(enamelin precursor) c4(222142) c5(222143, 222144, 222145, 222146, 222147, 222148, 222149, 222150, 222151) c6(222152, 222153, 222154) c7(gk, ka, blY, ki, al, bcw, jx, blZ, bma, bh, Ud, ai); #c1(ENC1) c2(XP_011541999) c3(ectoderm-neural cortex protein 1 isoform X1) c4(340467, 340468, 340469) c5(340470, 340471, 340472, 340473, 340474, 340475, 340476, 340477, 340478, 340479, 340480, 340481, 340482, 340483, 340484, 340485, 340486, 340487, 340488, 340489, 340490, 340491, 340492) c6( )c7(T, B, dB, bE, aT, bB, h, fr, K, cm, q, zn); #c1(ENDOG) c2(NP_004426) c3(endonuclease G, mitochondrial precursor) c4(146267) c5(146268, 146269, 146270, 146271, 146272, 146273) c6( ) c7(T, bP, aW, eu, ff, r, aT, cd, aA, fd, bl, q, en, bw, AO, bt, ae, B, ad); #c1(ENDOU) c2(NP_001165910) c3(poly(U)-specific endoribonuclease isoform 1 precursor) c4(67997, 67998) c5(67999, 68000, 68001, 68002, 68003, 68004, 68005, 68006) c6(68007, 68008) c7(Ei, U, aT, bO, bS, bK, K, ct, Z, he, ra, hq); #c1(ENDOU) c2(NP_001165911) c3(poly(U)-specific endoribonuclease isoform 3 precursor) c4(68009) c5(68010, 68011, 68012, 68013, 68014, 68015, 68016, 68017, 68018, 68019, 68020, 68021, 68022, 68023, 68024, 68025) c6(68026, 68027, 68028, 68029) c7(Ei, U, aT, bO, bS, bK, K, ct, Z, he, ra, hq); #c1(ENDOU) c2(NP_006016) c3(poly(U)-specific endoribonuclease isoform 2 precursor) c4(161195) c5(161196, 161197, 161198, 161199, 161200, 161201, 161202, 161203) c6( )c7(Ei, U, aT, bO, bS, bK, K, ct, Z, he, ra, hq); #c1(ENDOV) c2(NP_001158109) c3(endonuclease V isoform 2) c4(63141) c5(63142, 63143, 63144, 63145, 63146, 63147) c6( ) c7(CT, X, La, bC); #c1(ENDOV) c2(NP_001158110) c3(endonuclease V isoform 3) c4(63148) c5(63149, 63150, 63151, 63152, 63153, 63154) c6( )c7(CT, X, La, bC); #c1 (ENDOV) c2(NP_775898) c3(endonuclease V isoform 1) c4(250604) c5(250605, 250606, 250607, 250608, 250609, 250610) c6( )c7(CT, X, La, bC); #c1(ENG) c2(NP_000109) c3(endoglin isoform 2 precursor) c4(1845) c5(1846, 1847, 1848, 1849, 1850, 1851) c6( )c7(ID, T, aC, fR, pf, if, e, qx, al, eb, bB, lR, ci, ip, x, aE, K, hu, ly, aW, hl, LZ, g, hJ, eF, uC, bw, ou, y, iA, vA, vy, cj, uJ, Hb, m, iB, aw, ht, n, fr, qN, Cd, cd, br, bmb, kl, Cf, dL, dx, cY, wC, Ck, fe, lx, hL, at, pg, bD, it, B, dA, eR, De, aA, cz, Cg, Cb, q, o, er, aHu, qK, aT, dz, ae, Y, r, en, cl, t, Vq, iK, pb, amF, nM, ac, c, bt, kh, id, MH, CI, bs, ad, afb, BY, Jm, av, F, Cj, fb, nJ, d, BO, et, ff, eH, Ce, aF, si, iV, bz, es, bE, dX, aS, bC, lC, us); #c1(ENG) c2(NP_001108225) c3(endoglin isoform 1 precursor) c4(43169, 43170) c5(43171, 43172, 43173, 43174, 43175, 43176, 43177, 43178, 43179, 43180, 43181, 43182, 43183, 43184, 43185, 43186) c6( )c7(ID, T, aC, fR, pf, if, e, qx, al, eb, bB, lR, ci, ip, x, aE, K, hu, ly, aW, hl, LZ, g, hJ, eF, uC, bw, ou, y, iA, vA, vy, cj, uJ, Hb, m, iB, aw, ht, n, fr, qN, Cd, cd, br, bmb, kl, Cf, dL, dx, cY, wC, Ck, fe, lx, hL, at, pg, bD, it, B, dA, eR, De, aA, cz, Cg, Cb, q, o, er, aHu, qK, aT, dz, ae, Y, r, en, cl, t, Vq, iK, pb, amF, nM, ac, c, bt, kh, id, MH, CI, bs, ad, afb, BY, Jm, av, F, Cj, fb, nJ, d, BO, et, ff, eH, Ce, aF, si, iV, bz, es, bE, dX, aS, bC, lC, us); #c1(ENGASE) c2(NP_001036038) c3(cytosolic endo-beta-N-acetylglucosaminidase) c4(33784) c5(33785, 33786, 33787, 33788, 33789, 33790, 33791, 33792, 33793) c6( )c7(aW, T, y, aT); #c1(ENGASE) c2(XP_006722082) c3(cytosolic endo-beta-N-acetylglucosaminidase isoform X4) c4(294598) c5(294599, 294600, 294601, 294602, 294603, 294604) c6( )c7(aW, T, y, aT); #c1(ENH0) c2(NP_940975) c3(adropin precursor) c4(260230, 260231) c5(260232, 260233, 260234, 260235, 260236, 260237, 260238, 260239, 260240, 260241, 260242, 260243, 260244, 260245) c6(260246, 260247) c7(dq, v, qO); #c1(EN02) c2(NP_001966) c3(gamma-enolase) c4(122693, 122694) c5(122695, 122696, 122697, 122698, 122699, 122700, 122701, 122702, 122703, 122704, 122705, 122706, 122707, 122708, 122709, 122710, 122711, 122712, 122713, 122714, 122715, 122716, 122717, 122718, 122719, 122720, 122721) c6( )c7(T, aFG, jp, jd, jh, rf, v, ci, iJ, eW, ad, Nh, er, av, aaF, ft, Nn, SV, abg, aT, lG, cy, dL, vd, K, bq, H, ae, af, dx, awV, jf, iF, bmc, yo, d, lR, s, vT, UT, aW, je, fc, Pg, CO, f, cm, bz, bmd, ek, dS, ov, Qi, iM, jV, ac, eM, Ew, w, apl, cj, MJ, oQ, hh, bt, xg, iu, L); #c1(EN03) c2(NP_001180432) c3(beta-enolase isoform 2) c4(74045) c5(74046, 74047, 74048, 74049, 74050, 74051) c6( )c7(ck, lg, sO, bme, hl, km, jh, gE, vQ); #c1(EN03) c2(NP_443739) c3(beta-enolase isoform 1) c4( )c5(230073, 230074, 230075, 230076, 230077, 230078, 230079, 230080, 230081, 230082, 230083, 230084, 230085, 230086, 230087, 230088, 230089, 230090, 230091, 230092, 230093, 230094, 230095) c6( )c7(ck, lg, sO, bme, hl, km, jh, gE, vQ); #c1(EN03) c2(XP_005256578) c3(beta-enolase isoform X2) c4(274215, 274216, 274217, 274218) c5(274219, 274220, 274221, 274222, 274223) c6( )c7(ck, lg, sO, bme, hl, km, jh, gE, vQ); #c1(EN04) c2(NP_001229628) c3(enolase-like protein EN04) c4(84699) c5(84700, 84701, 84702, 84703, 84704, 84705) c6( )c7(gU); #c1(ENOPH1) c2(NP_067027) c3(enolase-phosphatase E1 isoform 1) c4(208517, 208518) c5(208519, 208520, 208521, 208522, 208523, 208524, 208525, 208526, 208527, 208528, 208529, 208530, 208531) c6( )c7(aio, ul, bmf, nA); #c1(ENOSF1) c2(NP_001119595) c3(mitochondrial enolase superfamily member 1 isoform rTSalpha) c4(44268) c5(44269, 44270, 44271, 44272, 44273, 44274) c6( )c7(T, dD, eq, q, hX, MR, jZ, aVA, aT, dV, ask, aVB, eu, apn, r, cl, aU, dr, ac, wE, hl, bm); #c1(ENOSF1) c2(NP_059982) c3(mitochondrial enolase superfamily member 1 isoform rTSbeta) c4(193470) c5(193471, 193472, 193473, 193474, 193475, 193476) c6( ) c7(T, dD, eq, q, hX, MR, jZ, aVA, aT, dV, ask, aVB, eu, apn, r, cl, aU, dr, ac, wE, hl, bm); #c1(ENOSF1) c2(NP_974487) c3(mitochondrial enolase superfamily member 1 isoform rTSgamma) c4(262726) c5(262727, 262728, 262729, 262730, 262731, 262732) c6( )c7(T, dD, eq, q, hX, MR, jZ, aVA, aT, dV, ask, aVB, eu, apn, r, cl, aU, dr, ac, wE, hl, bm); #c1(ENOSF1) c2(XP_005258175) c3(mitochondrial enolase superfamily member 1 isoform X18) c4( )c5(275536, 275537, 275538, 275539, 275540, 275541) c6( )c7(T, dD, eq, q, hX, MR, jZ, aVA, aT, dV, ask, aVB, eu, apn, r, cl, aU, dr, ac, wE, hl, bm); #c1(ENOSF1) c2(XP_011523981) c3(mitochondrial enolase superfamily member 1 isoform X3) c4(318529) c5( )c6( )c7(T, dD, eq, q, hX, MR, jZ, aVA, aT, dV, ask, aVB, eu, apn, r, cl, aU, dr, ac, wE, hl, bm); #c1(ENOSF1) c2(XP_011523982) c3(mitochondrial enolase superfamily member 1 isoform X4) c4(318530) c5( )c6( )c7(T, dD, eq, q, hX, MR, jZ, aVA, aT, dV, ask, aVB, eu, apn, r, cl, aU, dr, ac, wE, hl, bm); #c1(ENOSF1) c2(XP_011523984) c3(mitochondrial enolase superfamily member 1 isoform X6) c4( )c5(318531, 318532, 318533, 318534, 318535, 318536) c6( )c7(T, dD, eq, q, hX, MR, jZ, aVA, aT, dV, ask, aVB, eu, apn, r, cl, aU, dr, ac, wE, hl, bm); #c1(ENOX1) c2(NP_001121087) c3(ecto-NOX disulfide-thiol exchanger 1) c4(45173, 45174) c5(45175, 45176, 45177, 45178, 45179, 45180, 45181, 45182) c6( )c7(aDx, T, oe, y); #c1(ENOX1) c2(XP_011533427) c3(ecto-NOX disulfide-thiol exchanger 1 isoform X2) c4( )c5(329552, 329553) c6( )c7(aDx, T, oe, y); #c1(ENOX1) c2(XP_011533434) c3(ecto-NOX disulfide-thiol exchanger 1 isoform X3) c4( )c5(329554, 329555, 329556, 329557, 329558, 329559) c6( )c7(aDx, T, oe, y); #c1(ENOX2) c2(XP_011529546) c3(ecto-NOX disulfide-thiol exchanger 2 isoform X3) c4(325066, 325067, 325068) c5(325069, 325070, 325071, 325072, 325073, 325074, 325075) c6( )c7(bv, T, aF, aA); #c1(ENOX2) c2(XP_011529547) c3(ecto-NOX disulfide-thiol exchanger 2 isoform X4) c4(325076) c5(325077) c6( )c7(bv, T, aF, aA); #c1(ENOX2) c2(XP_011529552) c3(ecto-NOX disulfide-thiol exchanger 2 isoform X1) c4(325078) c5(325079, 325080, 325081, 325082, 325083, 325084, 325085, 325086, 325087, 325088, 325089) c6( )c7(bv, T, aF, aA); #c1(ENOX2) c2(XP_011529554) c3(ecto-NOX disulfide-thiol exchanger 2 isoform X2) c4( )c5(325090, 325091, 325092, 325093, 325094, 325095, 325096, 325097, 325098, 325099, 325100, 325101, 325102, 325103, 325104, 325105, 325106, 325107) c6( )c7(bv, T, aF, aA); #c1 (ENPEP) c2(NP_001968) c3(glutamyl aminopeptidase) c4(122722) c5(122723, 122724, 122725, 122726, 122727, 122728) c6( ) c7(dA, Fj, WA, rd, cV, bs, cz, n, bO, fr, qN, br, ad, aT, vw, h, Fg, ae, nJ, ayT, d, s, aW, ff, Hu, en, aoG, bw, bN, ck, es, rK, LS); #c1(ENPP1) c2(NP_006199) c3(ectonucleotide pyrophosphatase/phosphodiesterase family member 1) c4(162806) c5(162807, 162808, 162809, 162810, 162811, 162812) c6( )c7(bL, nZ, T, dD, AB, Ly, eR, aZ, ea, e, jx, ql, aw, Ay, CR, cz, aj, bmi, j, dG, v, eq, fo, nS, aY, amC, cd, J, ad, zn, dP, amB, zw, aE, lK, rC, oW, aT, dV, jy, dL, qM, ae, af, kl, d, mR, A, bmh, bmg, HY, aW, fc, rs, si, fd, bfH, AA, bmj, uC, dr, aDQ, bJ, amF, y, ac, rq, cO, bib, mg, asR, mf, dy, lS, ei, bfl, VK, fl); #c1(ENPP2) c2(NP_001035181) c3(ectonucleotide pyrophosphatase/phosphodiesterase family member 2 isoform 2 preproprotein) c4(32556) c5(32557, 32558, 32559, 32560, 32561, 32562, 32563, 32564) c6( )c7(T, DD, bmk, al, jx, eX, fd, cR, fr, ai, ad, Vp, hd, F, aT, eV, bq, ae, aW, rU, fQ, t, cm, LB, qJ, nB, dX, np, y, B); #c1(ENPP2) c2(NP_001124335) c3(ectonucleotide pyrophosphatase/phosphodiesterase family member 2 isoform 3 preproprotein) c4(48727) c5(48728, 48729, 48730, 48731, 48732, 48733) c6( )c7(T, DD, bmk, al, jx, eX, fd, cR, fr, ai, ad, Vp, hd, F, aT, eV, bq, ae, aW, rU, fQ, t, cm, LB, qJ, nB, dX, np, y, B); #c1(ENPP2) c2(NP_006200) c3(ectonucleotide pyrophosphatase/phosphodiesterase family member 2 isoform 1 preproprotein) c4(162813) c5(162814, 162815, 162816, 162817, 162818, 162819) c6( )c7(T, DD, bmk, al, jx, eX, fd, cR, fr, ai, ad, Vp, hd, F, aT, eV, bq, ae, aW, rU, fQ, t, cm, LB, qJ, nB, dX, np, y, B); #c1(ENPP2) c2(XP_006716647) c3 (ectonucleotide pyrophosphatase/phosphodiesterase family member 2 isoform X1) c4( )c5(290420) c6( )c7(T, DD, bmk, al, jx, eX, fd, cR, fr, ai, ad, Vp, hd, F, aT, eV, bq, ae, aW, rU, fQ, t, cm, LB, qJ, nB, dX, np, y, B); #c1(ENPP2) c2(XP_006716650) c3(ectonucleotide pyrophosphatase/phosphodiesterase family member 2 isoform X4) c4(290421) c5( )c6( )c7(T, DD, bmk, al, jx, eX, fd, cR, fr, ai, ad, Vp, hd, F, aT, eV, bq, ae, aW, rU, fQ, t, cm, LB, qJ, nB, dX, np, y, B); #c1(ENPP3) c2(NP_005012) c3(ectonucleotide pyrophosphatase/phosphodiesterase family member 3) c4(152133, 152134, 152135, 152136) c5(152137, 152138, 152139, 152140, 152141, 152142, 152143, 152144, 152145, 152146, 152147, 152148, 152149, 152150, 152151, 152152, 152153, 152154, 152155, 152156, 152157, 152158, 152159, 152160, 152161, 152162, 152163, 152164) c6( )c7(nZ, cT, T, bjX, e, X, bt, apG, aq, ci, bmj, [x, B, yR, hX); #c1 (ENPP5) c2(XP_005249317) c3 (ectonucleotide pyrophosphatase/phosphodiesterase family member 5 isoform XI) c4(269158) c5(269159, 269160, 269161, 269162, 269163, 269164, 269165, 269166, 269167, 269168, 269169, 269/70, 269171, 269/72) c6(269173, 269174, 269175, 269/76) c7(ad); #c1(ENPP7) c2(NP_848638) c3(ectonucleotide pyrophosphatase/phosphodiesterase family member 7 precursor) c4(254041) c5(254042, 254043, 254044, 254045, 254046, 254047) c6( )c7(bB, bE, X, ao); #c1(ENTHD2) c2(NP_653280) c3(AP-4 complex accessory subunit tepsin) c4(239490) c5(239491, 239492, 239493, 239494, 239495, 239496) c6( ) c7(cr); #c1(ENTHD2) c2(XP_005257124) c3(AP-4 complex accessory subunit tepsin isoform X5) c4( )c5(274703) c6( )c7(cr); #c1(ENTHD2) c2(XP_006721775) c3(AP-4 complex accessory subunit tepsin isoform X7) c4( )c5(294272, 294273, 294274, 294275, 294276, 294277) c6( )c7(cr); #c1(ENTHD2) c2(XP_011522660) c3(AP-4 complex accessory subunit tepsin isoform X6) c4(317222) c5( ) c6( )c7(cr); #c1(ENTPD1) c2(NP_001091645) c3(ectonucleoside triphosphate diphosphohydrolase 1 isoform 2) c4(38539, 38540, 38541, 38542) c5(38543, 38544, 38545, 38546, 38547, 38548, 38549, 38550, 38551, 38552, 38553, 38554) c6( )c7(bL, bM, T, eR, aD, aj, j, nA, C, mF, aY, V, jw, J, aE, bmm, Co, h, cy, ji, bq, pk, bml, Y, fe, vs, ek, il, ac, cj, gZ, aS, rH, pw, ei, gT); #c1(ENTPD1) c2(NP_001157650) c3(ectonucleoside triphosphate diphosphohydrolase 1 isoform 3) c4(62373) c5(62374, 62375, 62376, 62377, 62378, 62379) c6( ) c7(bL, bM, T, eR, aD, aj, j, nA, C, mF, aY, V, jw, J, aE, bmm, Co, h, cy, ji, bq, pk, bml, Y, fe, vs, ek, il, ac, cj, gZ, aS, rH, pw, ei, gT); #c1(ENTPD1) c2(NP_001157653) c3(ectonucleoside triphosphate diphosphohydrolase 1 isoform 5) c4( )c5(62380, 62381, 62382, 62383, 62384, 62385, 62386) c6( )c7(bL, bM, T, eR, aD, aj, j, nA, C, mF, aY, V, jw, J, aE, bmm, Co, h, cy, ji, bq, pk, bml, Y, fe, vs, ek, il, ac, cj, gZ, aS, rH, pw, ei, gT); #c1(ENTPD1) c2(NP_001157654) c3(ectonucleoside triphosphate diphosphohydrolase 1 isoform 6) c4( )c5(62387, 62388, 62389, 62390, 62391, 62392, 62393, 62394) c6( )c7(bL, bM, T, eR, aD, aj, j, nA, C, mF, aY, V, jw, J, aE, bmm, Co, h, cy, ji, bq, pk, bml, Y, fe, vs, ek, il, ac, cj, gZ, aS, rH, pw, ei, gT); #c1(ENTPD1) c2(NP_001767) c3(ectonucleoside triphosphate diphosphohydrolase 1 isoform 1) c4(120956) c5(120957, 120958, 120959, 120960, 120961, 120962, 120963) c6( )c7(bL, bM, T, eR, aD, aj, j, nA, C, mF, aY, V, jw, J, aE, bmm, Co, h, cy, ji, bq, pk, bml, Y, fe, vs, ek, il, ac, cj, gZ, aS, rH, pw, ei, gT); #c1(ENTPD2) c2(NP_001237) c3(ectonucleoside triphosphate diphosphohydrolase 2 isoform 2) c4(87886) c5(87887, 87888, 87889, 87890, 87891, 87892) c6( )c7(aE, Y, cz, fQ, aq, B, bc); #c1(ENTPD2) c2(NP_982293) c3(ectonucleoside triphosphate diphosphohydrolase 2 isoform 1) c4(263301) c5(263302, 263303, 263304, 263305, 263306, 263307) c6( )c7(aE, Y, cz, fQ, aq, B, bc); #c1(ENTPD4) c2(NP_001122402) c3(ectonucleoside triphosphate diphosphohydrolase 4 isoform b) c4(46775, 46776) c5(46777, 46778, 46779, 46780, 46781, 46782, 46783, 46784, 46785, 46786, 46787, 46788) c6( )c7(T); #c1(ENTPD4) c2(NP_004892) c3(ectonucleoside triphosphate diphosphohydrolase 4 isoform a) c4(150608) c5(150609, 150610, 150611, 150612, 150613, 150614) c6( )c7(T); #c1(ENTPD5) c2(XP_006720387) c3(ectonucleoside triphosphate diphosphohydrolase 5 isoform X1) c4(293504) c5(293505, 293506, 293507, 293508, 293509, 293510) c6( )c7(GI, BI, T, vT, aT, fZ, r, Ej, Lb, K, bu, rA, ae, q, ad); #c1(ENTPD6) c2(NP_001107561) c3(ectonucleoside triphosphate diphosphohydrolase 6 isoform 2) c4(42836) c5(42837, 42838, 42839, 42840, 42841, 42842) c6( )c7(wu, wm); #c1(ENTPD6) c2(XP_005260938) c3(ectonucleoside triphosphate diphosphohydrolase 6 isoform X4) c4(276988, 276989) c5(276990) c6( )c7(wu, wm); #c1(ENTPD6) c2(XP_005260939) c3(ectonucleoside triphosphate diphosphohydrolase 6 isoform X5) c4(276991) c5(276992) c6( )c7(wu, wm); #c1(ENTPD6) c2(XP_005260940) c3(ectonucleoside triphosphate diphosphohydrolase 6 isoform X6) c4( )c5(276993, 276994, 276995, 276996, 276997, 276998, 276999, 277000, 277001, 277002, 277003) c6( )c7(wu, wm); #c1(ENTPD6) c2(XP_005260941) c3(ectonucleoside triphosphate diphosphohydrolase 6 isoform X7) c4(277004) c5(277005, 277006, 277007, 277008, 277009, 277010) c6( )c7(wu, wm); #c1(ENTPD6) c2(XP_005260942) c3(ectonucleoside triphosphate diphosphohydrolase 6 isoform X8) c4(277011) c5(277012, 277013, 277014, 277015, 277016, 277017) c6( )c7(wu, wm); #c1(ENTPD6) c2(XP_011527698) c3 (ectonucleoside triphosphate diphosphohydrolase 6 isoform X1) c4(322636) c5( )c6( )c7(wu, wm); #c1(ENTPD6) c2(XP_011527699) c3(ectonucleoside triphosphate diphosphohydrolase 6 isoform X2) c4(322637) c5( )c6( )c7(wu, wm); #c1(ENTPD6) c2(XP_011527700) c3(ectonucleoside triphosphate diphosphohydrolase 6 isoform X3) c4(322638, 322639) c5( )c6( )c7(wu, wm); #c1(ENTPD6) c2(XP_011527701) c3(ectonucleoside triphosphate diphosphohydrolase 6 isoform X9) c4(322640, 322641, 322642) c5(322643, 322644, 322645, 322646, 322647, 322648) c6( )c7(wu, wm); #c1(ENTPD6) c2(XP_011527702) c3(ectonucleoside triphosphate diphosphohydrolase 6 isoform X10) c4(322649) c5(322650, 322651, 322652, 322653, 322654, 322655, 322656, 322657, 322658, 322659, 322660, 322661, 322662, 322663, 322664, 322665, 322666, 322667) c6( )c7(wu, wm); #c1(ENTPD6) c2(XP_011527703) c3(ectonucleoside triphosphate diphosphohydrolase 6 isoform X11) c4( )c5(322668, 322669, 322670, 322671, 322672, 322673, 322674, 322675, 322676, 322677, 322678, 322679, 322680, 322681, 322682, 322683, 322684) c6( )c7(wu, wm); #c1(ENTPD7) c2(NP_065087) c3(ectonucleoside triphosphate diphosphohydrolase 7) c4(203831) c5(203832, 203833, 203834, 203835, 203836, 203837) c6( )c7(af); #c1(EDGT) c2(NP_001265618) c3(EGF domain-specific O-linked N-acetylglucosamine transferase isoform a precursor) c4(98994, 98995, 98996, 98997) c5(98998, 98999, 99000, 99001, 99002, 99003, 99004, 99005, 99006, 99007, 99008, 99009, 99010, 99011, 99012, 99013, 99014, 99015, 99016, 99017, 99018, 99019, 99020, 99021) c6(99022, 99023, 99024, 99025, 99026, 99027) c7(afG, bmn); #c1(EOGT) c2(NP_775925) c3(EGF domain-specific O-linked N-acetylglucosamine transferase isoform b precursor) c4(250725) c5(250726, 250727, 250728, 250729, 250730, 250731, 250732, 250733) c6(250734, 250735) c7(afG, bmn); #c1(EOGT) c2(XP_011531904) c3(EGF domain-specific O-linked N-acetylglucosamine transferase isoform X2) c4(327940) c5( )c6( )c7(afG, bmn); #c1(EOGT) c2(XP_011531907) c3(EGF domain-specific O-linked N-acetylglucosamine transferase isoform X5) c4( )c5(327941, 327942, 327943, 327944, 327945, 327946, 327947, 327948, 327949, 327950, 327951, 327952, 327953, 327954, 327955, 327956) c6(327957, 327958, 327959, 327960) c7(afG, bmn); #c1(EOMES) c2(NP_001265111) c3(eomesodermin homolog isoform 1) c4(97788, 97789, 97790) c5(97791, 97792, 97793, 97794, 97795, 97796, 97797, 97798, 97799, 97800, 97801, 97802) c6( )c7(c, wu, CM, KY, Ej, lo, dl, fr, wm, QK, V, eL, cd); #c1 (EOMES) c2(NP_001265112) c3(comesodermin homolog isoform 3) c4( )c5(97803, 97804, 97805, 97806, 97807, 97808) c6( )c7(c, wu, CM, KY, Ej, lo, dl, fr, wm, QK, V, eL, cd); #c1(EOMES) c2(NP_005433) c3(eomesodermin homolog isoform 2) c4(156136) c5(156137, 156138, 156139, 156140, 156141, 156142, 156143) c6( )c7(c, wu, CM, KY, Ej, lo, dL, fr, wm, QK, V, eL, cd); #c1(EP300) c2(NP_001420) c3(histone acetyltransferase p300) c4(117723) c5( )c6( )c7(T, fM, id, jh, cz, bB, v, ci, C, q, ad, x, ft, bmp, qK, aT, lG, dz, dL, K, jo, bq, ae, bmo, d, mR, il, aCM, iZ, aW, Y, ff, fc, eZ, r, vs, aF, en, cl, t, bw, bz, aU, ck, Hw, eM, lp, bE, QQ, DW, vy, hL, gZ, Lo, k, m, cP, Km, bt, it, B); #c1(EP300) c2(XP_006724228) c3(histone acetyltransferase p300 isoform X1) c4( )c5(295863, 295864, 295865, 295866, 295867, 295868, 295869, 295870) c6( )c7(T, fM, id, jh, cz, bB, v, ci, C, q, ad, x, ft, bmp, qK, aT, lG, dz, dL, K, jo, bq, ae, bmo, d, mR, il, aCM, iZ, aW, Y, ff, fc, eZ, r, vs, aF, en, cl, t, bw, bz, aU, ck, Hw, eM, lp, bE, QQ, DW, vy, hL, gZ, Lo, k, m, cP, Km, bt, it, B); #c1(EP400) c2(NP_056224) c3(E1A-binding protein p400) c4(186489, 186490, 186491, 186492, 186493, 186494, 186495, 186496, 186497, 186498, 186499, 186500, 186501, 186502) c5(186503, 186504, 186505, 186506, 186507, 186508, 186509, 186510, 186511, 186512, 186513, 186514, 186515, 186516, 186517, 186518, 186519, 186520, 186521, 186522, 186523, 186524, 186525, 186526, 186527, 186528, 186529, 186530, 186531, 186532, 186533, 186534, 186535, 186536, 186537, 186538, 186539, 186540, 186541, 186542, 186543, 186544, 186545, 186546, 186547, 186548, 186549, 186550, 186551, 186552, 186553, 186554, 186555, 186556, 186557, 186558, 186559, 186560, 186561, 186562, 186563, 186564, 186565, 186566, 186567, 186568, 186569, 186570, 186571, 186572, 186573, 186574, 186575, 186576, 186577, 186578, 186579, 186580, 186581, 186582, 186583, 186584, 186585, 186586, 186587, 186588, 186589) c6( )c7(B, bP, jE, bmq, bt, q); #c1(EPAS1) c2(NP_001421) c3(endothelial PAS domain-containing protein 1) c4(117724, 117725) c5(117726, 117727, 117728, 117729, 117730, 117731, 117732, 117733, 117734, 117735, 117736, 117737, 117738) c6( ) c7(gx, T, kh, sw, if, jh, aA, bB, hy, j, ci, fr, bkU, q, ad, eA, Hr, x, iu, oT, ft, py, aE, yw, oW, aT, dl, bmr, K, sR, pk, ae, azh, eu, DA, aW, Y, anx, ff, cr, r, bmu, aq, vt, aF, zC, en, g, as, LV, t, fq, bz, cm, iR, ck, es, ou, iO, y, kP, eM, iA, bE, X, bmt, k, Hb, m, GF, lC, bt, bms, B, VB); #c1(EPB 4ILI) c2(NP_001245258) c3 (band 4,1-like protein 1 isoform c) c4(92909) c5(92910, 92911, 92912, 92913, 92914, 92915, 92916) c6( )c7(bmv, n, T); #c1(EPB4ILI) c2(NP_001245259) c3(band 4,1-like protein 1 isoform d) c4(92917, 92918) c5(92919, 92920, 92921, 92922, 92923, 92924, 92925, 92926, 92927, 92928, 92929, 92930, 92931, 92932, 92933, 92934, 92935, 92936, 92937, 92938, 92939, 92940, 92941, 92942, 92943) c6( )c7(bmv, n, T); #c1(EPB4IL1) c2(NP_036288) c3(band 4,1-like protein 1 isoform a) c4(172854) c5(172855, 172856, 172857, 172858, 172859, 172860) c6( )c7(bmv, n, T); #c1(EPB4ILI) c2(XP_011526966) c3(band 4,1-like protein 1 isoform X1) c4(321268) c5( )c6( )c7(bmv, n, T); #c1(EPB4ILI) c2(XP_011526969) c3(band 4,1-like protein 1 isoform X4) c4( )c5(321269, 321270, 321271, 321272, 321273, 321274) c6( )c7(bmv, n, T); #c1(EPB4ILI) c2(XP_011526970) c3(band 4,1-like protein 1 isoform X5) c4(321275, 321276) c5( )c6( )c7(bmv, n, T); #c1(EPB4ILI) c2(XP_011526975) c3(band 4,1-like protein 1 isoform X7) c4(321277) c5(321278) c6( ) c7(bmv, n, T); #c1(EPB4ILI) c2(XP_011526979) c3(band 4,1-like protein 1 isoform X11) c4( )c5(321279, 321280, 321281, 321282, 321283) c6( )c7(bmv, n, T); #c1(EPB4ILI) c2(XP_011526982) c3(band 4,1-like protein 1 isoform X14) c4(321284) c5(321285, 321286, 321287, 321288, 321289, 321290, 321291, 321292, 321293, 321294) c6( )c7(bmv, n, T); #c1(EPB4ILI) c2(XP_011526983) c3(band 4,1-like protein 1 isoform X15) c4(321295) c5( )c6( )c7(bmv, n, T); #c1(EPB4ILI) c2(XP_011526985) c3(band 4,1-like protein 1 isoform X17) c4(321296) c5(321297, 321298, 321299, 321300, 321301, 321302) c6( )c7(bmv, n, T); #c1(EPB4ILI) c2(XP_011526986) c3(band 4,1-like protein 1 isoform X18) c4(321303) c5(321304, 321305, 321306, 321307, 321308, 321309, 321310, 321311, 321312, 321313) c6( )c7(bmv, n, T); #c1(EPB4ILI) c2(XP_011526987) c3(band 4,1-like protein 1 isoform X19) c4(321314) c5( )c6( )c7(bmv, n, T); #c1(EPB4ILI) c2(XP_011526988) c3(band 4,1-like protein 1 isoform X20) c4(321315, 321316) c5(321317, 321318, 321319, 321320, 321321, 321322, 321323, 321324, 321325, 321326, 321327, 321328, 321329, 321330, 321331, 321332, 321333, 321334, 321335, 321336, 321337, 321338, 321339, 321340, 321341, 321342) c6( )c7(bmv, n, T); #c1(EPB4IL2) c2(NP_001129026) c3(band 4,1-like protein 2 isoform b) c4(50636) c5(50637, 50638, 50639, 50640, 50641, 50642, 50643, 50644, 50645, 50646, 50647, 50648, 50649, 50650, 50651, 50652, 50653, 50654, 50655, 50656, 50657, 50658, 50659, 50660, 50661, 50662, 50663, 50664, 50665, 50666, 50667, 50668, 50669, 50670, 50671, 50672, 50673, 50674, 50675, 50676) c6( )c7(EQ, pN); #c1(EPB41L2) c2(NP_001422) c3(band 4,1-like protein 2 isoform a) c4(117739, 117740) c5(117741, 117742, 117743, 117744, 117745, 117746, 117747, 117748, 117749, 117750, 117751, 117752, 117753, 117754, 117755, 117756) c6( ) c7(EQ, pN); #c1(EPB4IL2) c2(XP_006715425) c3(band 4,1-like protein 2 isoform X10) c4(289531) c5( )c6( )c7(EQ, pN); #c1(EPB4IL2) c2(XP_011533824) c3(band 4,1-like protein 2 isoform X3) c4(329982) c5( )c6( )c7(EQ, pN); #c1(EPB4IL2) c2(XP_011533826) c3(band 4,1-like protein 2 isoform X5) c4(329983, 329984) c5(329985, 329986, 329987, 329988, 329989, 329990, 329991, 329992, 329993, 329994, 329995, 329996) c6( )c7(EQ, pN); #c1(EPB4IL2) c2(XP_011533832) c3(band 4,1-like protein 2 isoform X1) c4(329997) c5(329998, 329999, 330000, 330001, 330002, 330003, 330004, 330005, 330006, 330007, 330008, 330009) c6( )c7(EQ, pN); #c1(EPB4IL2) c2(XP_011533833) c3(band 4,1-like protein 2 isoform X11) c4(330010, 330011) c5( )c6( )c7(EQ, pN); #c1(EPB4IL2) c2(XP_011533834) c3(band 4,1-like protein 2 isoform X12) c4( )c5(330012) c6( )c7(EQ, pN); #c1(EPB4IL2) c2(XP_011533835) c3(band 4,1-like protein 2 isoform X13) c4(330013) c5( )c6( )c7(EQ, pN); #c1(EPB4IL2) c2(XP_011533836) c3(band 4,1-like protein 2 isoform X2) c4(330014, 330015) c5(330016, 330017, 330018, 330019, 330020, 330021, 330022, 330023, 330024, 330025) c6( )c7(EQ, pN); #c1(EPB4IL3) c2(NP_001268462) c3(band 4,1-like protein 3 isoform 2) c4( )c5(99977, 99978, 99979, 99980, 99981, 99982) c6( )c7(T, oe, vr, j, ci, fr, ad, ip, iC, av, ft, py, Fo, aT, ae, aiV, aW, g, zE, t, bz, Ew, c, eM, dB, m, iu); #c1(EPB4IL3) c2(NP_001268463) c3(band 4,1-like protein 3 isoform 3) c4(99983, 99984) c5(99985, 99986, 99987, 99988, 99989, 99990, 99991, 99992, 99993) c6( )c7(T, oe, vr, j, ci, fr, ad, ip, iC, av, ft, py, Fo, aT, ae, aiV, aW, g, zE, t, bz, Ew, c, eM, dB, m, iu); #c1(EPB4IL3) c2(NP_001268464) c3(band 4,1-like protein 3 isoform 4) c4( )c5(99994, 99995, 99996, 99997, 99998, 99999, 100000, 100001) c6( )c7(T, oe, vr, j, ci, fr, ad, ip, iC, av, ft, py, Fo, aT, ae, aiV, aW, g, zE, t, bz, Ew, c, eM, dB, m, iu); #c1(EPB41L3) c2(NP_036439) c3(band 4,1-like protein 3 isoform 1) c4(173874) c5(173875, 173876, 173877, 173878, 173879, 173880, 173881, 173882, 173883, 173884) c6( )c7(T, oe, vr, j, ci, fr, ad, ip, iC, av, ft, py, Fo, aT, ae, aiV, aW, g, zE, t, bz, Ew, c, eM, dB, m, iu); #c1(EPB4IL3) c2(XP_011523920) c3(band 4,1-like protein 3 isoform X9) c4(318444) c5( )c6( )c7(T, De, vr, j, ci, fr, ad, ip, iC, av, ft, py, Fo, aT, ae, aiV, aW, g, zE, t, bz, Ew, c, eM, dB, m, iu); #c1(EPB41L3) c2(XP_011523925) c3(band 4,1-like protein 3 isoform X14) c4(318445) c5( )c6( )c7(T, oe, vr, j, ci, fr, ad, ip, iC, av, ft, py, Fo, aT, ae, aiV, aW, g, zE, t, bz, Ew, c, eM, dB, m, iu); #c1(EPB4IL3) c2(XP_011523926) c3(band 4,1-like protein 3 isoform X15) c4(318446) c5(318447, 318448, 318449, 318450, 318451, 318452) c6( )c7(T, oe, vr, j, ci, fr, ad, ip, iC, av, ft, py, Fo, aT, ae, aiV, aW, g, zE, t, bz, Ew, c, eM, dB, m, iu); #c1(EPB41L3) c2(XP_011523928) c3(band 4,1-like protein 3 isoform X17) c4( )c5(318453, 318454, 318455, 318456, 318457, 318458) c6( )c7(T, oe, vr, j, ci, fr, ad, ip, iC, av, ft, py, Fo, aT, ae, aiV, aW, g, zE, t, bz, Ew, c, eM, dB, m, iu); #c1(EPB4IL3) c2(XP_011523930) c3(band 4,1-like protein 3 isoform X19) c4(318459) c5( )c6( )c7(T, oe, vr, j, ci, fr, ad, ip, iC, av, ft, py, Fo, aT, ae, aiV, aW, g, zE, t, bz, Ew, c, eM, dB, m, iu); #c1(EPB4IL3) c2(XP_011523939) c3(band 4,1-like protein 3 isoform X28) c4( )c5(318460, 318461, 318462, 318463, 318464) c6( )c7(T, oe, vr, j, ci, fr, ad, ip, iC, av, ft, py, Fo, aT, ae, aiV, aW, g, zE, t, bz, Ew, c, eM, dB, m, iu); #c1(EPB41L4A) c2(NP_071423) c3(band 4,1-like protein 4A) c4(211590) c5(211591, 211592, 211593, 211594, 211595, 211596) c6( )c7(W); #c1(EPB41L4B) c2(NP_060894) c3(band 4,1-like protein 4B isoform 1) c4(198432, 198433) c5(198434, 198435, 198436, 198437, 198438, 198439, 198440, 198441, 198442, 198443, 198444, 198445) c6( )c7(T, aW, aT, n, ae, ad); #c1(EPB4IL4B) c2(NP_061987) c3(band 4,1-like protein 4B isoform 2) c4(201774, 201775) c5(201776, 201777, 201778, 201779, 201780, 201781, 201782, 201783, 201784, 201785, 201786, 201787) c6( )c7(T, aW, aT, n, ae, ad); #c1(EPB42) c2(NP_001107606) c3(erythrocyte membrane protein band 4.2 isoform 2) c4(42903, 42904) c5(42905, 42906, 42907, 42908, 42909, 42910, 42911, 42912, 42913, 42914, 42915, 42916) c6( )c7(CI, Fm, VJ, S, bmw); #c1(EPB42) c2(XP_011519653) c3(erythrocyte membrane protein band 4.2 isoform X1) c4(314318) c5(314319, 314320, 314321, 314322, 314323, 314324) c6( )c7(CI, Fm, VJ, S, bmw); #c1(EPB42) c2(XP_011519654) c3(erythrocyte membrane protein band 4.2 isoform X2) c4(314325) c5(314326, 314327, 314328, 314329, 314330, 314331) c6( )c7(CI, Fm, VJ, S, bmw); #c1(EPC1) c2(NP_001258933) c3(enhancer of polycomb homolog 1 isoform b) c4(96807) c5(96808, 96809, 96810, 96811, 96812, 96813) c6( )c7(ck, je, iM, afS, Th, bt); #c1(EPC1) c2(NP_001269320) c3(enhancer of polycomb homolog 1 isoform d) c4(101118) c5(101119, 101120, 101121, 101122, 101123, 101124) c6( )c7(ck, je, iM, afS, Th, bt); #c1(EPC1) c2(NP_079485) c3(enhancer of polycomb homolog 1 isoform a) c4(218317) c5(218318, 218319, 218320, 218321, 218322, 218323) c6( )c7(ck, je, iM, afS, Th, bt); #c1(EPC2) c2(NP_056445) c3(enhancer of polycomb homolog 2) c4(187573) c5(187574, 187575, 187576, 187577, 187578, 187579) c6( )c7(ck, ht, bt, it, af); #c1(EPCAM) c2(NP_002345) c3(epithelial cell adhesion molecule precursor) c4(127690) c5(127691, 127692, 127693, 127694, 127695, 127696) c6( )c7(T, aC, pf, if, fd, bB, ci, aE, K, hu, eL, xQ, zQ, aW, LZ, nY, NR, g, eM, nB, m, ht, NT, n, fr, qN, bmA, zP, eW, b, ju, dL, PX, aq, kp, kk, iO, bP, hL, nz, it, B, dA, KR, bmz, jx, aA, hz, bmx, q, mj, aT, rO, Fx, ae, s, vT, NE, oV, Y, bmy, r, en, cl, t, iK, nM, arh, OH, ad, fU, av, hd, db, NS, oW, ael, nJ, BO, ff, aF, aot, iR, bz, bE, X, i, iu); #c1(EPDR1) c2(NP_060019) c3 (mammalian ependymin-related protein 1 isoform 1 precursor) c4(193689, 193690) c5(193691, 193692, 193693, 193694, 193695, 193696, 193697, 193698, 193699, 193700, 193701, 193702, 193703, 193704, 193705, 193706) c6(193707, 193708, 193709, 193710) c7(aw, ad); #c1(EPG5) c2(NP_066015) c3(ectopic P granules protein 5 homolog) c4(206730, 206731, 206732) c5(206733, 206734, 206735, 206736, 206737, 206738, 206739, 206740, 206741, 206742, 206743, 206744, 206745, 206746, 206747, 206748, 206749, 206750) c6( )c7(T, cc, aW, oV, ff, ht, aT, r, fZ, en, q, bmB); #c1(EPGN) c2(NP_001257918) c3(epigen isoform 1 precursor) c4(95803) c5(95804, 95805, 95806, 95807, 95808, 95809, 95810, 95811) c6(95812, 95813) c7(fq, qe, ER); #c1(EPGN) c2(XP_005265723) c3(epigen isoform X2) c4(279676) c5(279677, 279678, 279679, 279680, 279681, 279682, 279683, 279684) c6(279685, 279686) c7(fq, qe, ER); #c1(EPHA1) c2(NP_005223) c3(ephrin type-A receptor 1 precursor) c4(154194) c5(154195, 154196, 154197, 154198, 154199, 154200) c6( )c7(T, kh, fM, yk, NT, qy, fZ, bB, jh, ci, bjz, Do, q, eA, x, iu, bmC, aT, hn, af, d, s, aW, Y, ff, r, aq, yq, en, g, Qt, zV, c, bE, hk, gX, cj, k, hE, B, y); #c1(EPHA3) c2(NP_005224) c3(ephrin type-A receptor 3 isoform a precursor) c4(154201) c5(154202, 154203, 154204, 154205, 154206, 154207) c6( )c7(ll, cZ, T, aC, ha, oA, akz, aA, bB, ajM, jh, Vd, Kh, ci, fr, hF, eA, akE, lF, bv, av, aT, h, hn, K, dd, pk, yj, mu, jf, nP, d, aW, Y, lW, eH, akB, aF, g, eF, t, cm, bz, HT, c, bE, bmD, afd, hr, k, m, bmE, zB, B, EB); #c1(EPHA3) c2(NP_872585) c3(ephrin type-A receptor 3 isoform b precursor) c4(256429) c5(256430, 256431, 256432, 256433, 256434, 256435) c6( )c7(lL, cZ, T, aC, ha, oA, akz, aA, bB, ajM, jh, Vd, Kh, ci, fr, hF, eA, akE, lF, bv, av, aT, h, hn, K, dd, pk, yj, mu, jf, nP, d, aW, Y, lW, eH, akB, aF, g, eF, t, cm, bz, HT, c, bE, bmD, afd, hr, k, m, bmE, zB, B, EB); #c1(EPHA4) c2(NP_001291465) c3(ephrin type-A receptor 4 isoform a precursor) c4(116117, 116118, 116119, 116120, 116121) c5(116122, 116123, 116124, 116125, 116126, 116127, 116128, 116129, 116130, 116131, 116132, 116133, 116134, 116135, 116136, 116137, 116138) c6( )c7(T, uD, ci, eA, ad, q, aE, h, lQ, an, bmG, ae, bmF, cp, d, Y, hl, r, kp, g, bz, Hv, iq, eU, c, iu); #c1(EPHA4) c2(NP_001291466) c3(ephrin type-A receptor 4 isoform b) c4(116139) c5(116140, 116141, 116142, 116143, 116144, 116145) c6( )c7(T, uO, ci, eA, ad, q, aE, h, lQ, an, bmG, ae, bmF, cp, d, Y, hl, r, kp, g, bz, Hv, iq, eU, c, iu); #c1(EPHA4) c2(XP_005246431) c3(ephrin type-A receptor 4 isoform X1) c4( )c5(267100, 267101, 267102, 267103, 267104, 267105, 267106, 267107, 267108, 267109, 267110, 267111) c6( )c7(T, ul, ci, eA, ad, q, aE, h, lQ, an, bmG, ae, bmF, cp, d, Y, hl, r, kp, g, bz, Hv, iq, eU, c, iu); #c1(EPHA5) c2(NP_001268694) c3(ephrin type-A receptor 5 isoform c) c4(100140) c5( )c6( )c7(Pt, aW, BA, Pg, aT); #c1(EPHA5) c2(NP_001268696) c3(ephrin type-A receptor 5 isoform e) c4( )c5(100141, 100142, 100143, 100144, 100145, 100146) c6( )c7(Pt, aW, eA, Pg, aT); #c1(EPHA5) c2(NP_004430) c3(ephrin type-A receptor 5 isoform a) c4(146274) c5(146275, 146276, 146277, 146278, 146279, 146280) c6( )c7(Pt, aW, eA, Pg, aT); #c1(EPHA5) c2(NP_872272) c3(ephrin type-A receptor 5 isoform b) c4(255989) c5(255990, 255991, 255992, 255993, 255994, 255995) c6( )c7(Pt, aW, eA, Pg, aT); #c1(EPHA6) c2(NP_001073917) c3(ephrin type-A receptor 6 isoform a) c4(36513) c5(36514, 36515, 36516, 36517, 36518, 36519) c6( )c7(v, j, agl); #c1(EPHAG) c2(NP_001265229) c3(ephrin type-A receptor 6 isoform c) c4(98006) c5( )c6( )c7(v, j, agl); #c1(EPHAG) c2(NP_001265230) c3(ephrin type-A receptor 6 isoform d) c4(98007) c5(98008, 98009, 98010, 98011, 98012, 98013) c6( )c7(v, j, agl); #c1(EPHA6) c2(NP_775926) c3(ephrin type-A receptor 6 isoform b) c4(250736) c5(250737, 250738, 250739, 250740, 250741, 250742, 250743, 250744, 250745, 250746, 250747) c6( )c7(v, j, agl); #c1(EPHAG) c2(XP_006713655) c3(ephrin type-A receptor 6 isoform X1) c4( )c5(287271) c6( )c7(v, j, agl); #c1(EPHA7) c2(NP_001275559) c3(ephrin type-A receptor 7 isoform 3 precursor) c4( )c5(106843, 106844, 106845, 106846, 106847, 106848) c6( )c7(ae, T, ft, d, fM, bE, eX, ul, r, bB, eV, jh, ci, hF, bmG, bz, q, ad); #c1(EPHA7) c2(NP_004431) c3(ephrin type-A receptor 7 isoform 1 precursor) c4(146281, 146282) c5(146283, 146284, 146285, 146286, 146287, 146288) c6( )c7(ae, T, ft, d, fM, bE, ex, uD, r, bB, eV, jh, ci, hF, bmG, bz, q, ad); #c1(EPHB1) c2(NP_004432) c3(ephrin type-B receptor 1 precursor) c4(146289, 146290) c5(146291, 146292, 146293, 146294, 146295, 146296) c6( )c7(bL, T, pY, ht, DV, bO, aA, cz, oR, jh, bS, j, ct, aBl, ci, ww, q, eA, ft, afm, iX, pX, an, qE, blD, tb, ff, r, aF, en, Z, Qt, aFA, ok, Ew, ac, cA, eB, VB, uz, yH, y, jd, Fl, B, hv); #c1(EPHB1) c2(XP_011510843) c3(ephrin type-B receptor 1 isoform X2) c4( )c5(301297, 301298, 301299, 301300, 301301, 301302) c6( )c7(bL, T, pY, ht, DV, bO, aA, cz, oR, jh, bS, j, ct, aBl, ci, ww, q, eA, ft, afm, iX, pX, an, qE, blD, tb, ff, r, aF, en, Z, Qt, aFA, ok, Ew, ac, cA, eB, VB, uz, yH, y, jd, Fl, B, hv); #c1(EPHB3) c2(NP_004434) c3(ephrin type-B receptor 3 precursor) c4(146297) c5(146298, 146299, 146300, 146301, 146302, 146303) c6( )c7(bF, T, s, pN, Qu, eM, ff, r, ci, en, asb, q); #c1(EPHB4) c2(NP_004435) c3(ephrin type-B receptor 4 precursor) c4(146304, 146305) c5(146306, 146307, 146308, 146309, 146310, 146311, 146312, 146313, 146314, 146315, 146316, 146317) c6( )c7(T, jh, aA, bB, ci, qN, Cb, tK, q, ad, eA, x, av, ib, aE, kl, aT, dl, hu, ae, vl, eL, De, qw, A, lR, LC, aW, ff, r, aq, aF, yq, en, cl, t, iK, bz, ck, bF, nM, bE, dy, m, k, it, B); #c1(EPHBG) c2(NP_001267724) c3(ephrin type-B receptor 6 isoform b) c4(99665) c5( )c6( )c7(T, n, aj, Gn, J, hX, iu, aT, h, aW, Y, oV, apn, aq, Ej, cm, bz, iO, eM, cj, PV, rH, im, rA, B); #c1(EPHB6) c2(XP_011544811) c3 (ephrin type-B receptor 6 isoform X1) c4(343837) c5(343838, 343839, 343840, 343841, 343842, 343843, 343844, 343845, 343846, 343847, 343848, 343849, 343850, 343851, 343852, 343853) c6( )c7(T, n, aj, Gn, J, hX, iu, aT, h, aW, Y, oV, apn, aq, Ej, cm, bz, iO, eM, cj, PV, rH, im, rA, B); #c1(EPHX1) c2(NP_000 μl) c3(epoxide hydrolase 1) c4(1852, 1853, 1854) c5(1855, 1856, 1857, 1858, 1859, 1860, 1861, 1862, 1863, 1864, 1865, 1866, 1867, 1868, 1869, 1870, 1871, 1872, 1873, 1874, 1875, 1876) c6( )c7(bM, T, fR, if, hE, eb, bB, fd, ci, fw, sD, x, K, hu, eL, xa, aAX, zQ, aW, Az, hl, aaD, uC, yd, eM, Gs, cj, uo, k, m, sl, aw, ht, aBx, qN, pM, zP, zw, eV, Gw, dl, ny, aq, W, dB, Qu, at, ut, eQ, it, B, bL, EM, jx, eX, aA, hy, j, bX, q, aT, an, ae, kl, s, r, en, t, ck, fD, rA, bt, anh, hS, Gl, am, id, iW, ha, jh, Gn, jE, fu, ad, hX, dP, av, F, ib, oW, lQ, ff, af, bz, bmH, TA, gk, Zi, bE, pP, Gu, lC, X, rk); #c1(EPHX2) c2(NP_001243411) c3(bifunctional epoxide hydrolase 2 isoform b) c4( )c5(90713, 90714, 90715, 90716, 90717, 90718, 90719, 90720, 90721, 90722, 90723) c6( )c7(bL, bM, dD, eR, fR, s, mr, ea, iW, e, cz, n, BH, aj, j, hy, eq, cd, J, ad, dY, ec, iX, M, dL, ey, lQ, ae, eu, BN, cH, r, iy, eK, cl, ac, hc, dh, cj, uo, at, ut, lS, ei, lC, ne); #c1(EPHX2) c2(NP_001970) c3(bifunctional epoxide hydrolase 2 isoform a) c4(122729, 122730, 122731, 122732) c5(122733, 122734, 122735, 122736, 122737, 122738, 122739, 122740, 122741, 122742, 122743, 122744, 122745) c6( )c7(bL, bM, dD, eR, fR, sQ, mr, ea, iW, e, cz, n, BH, aj, j, hy, eq, cd, J, ad, dY, ec, iX, M, dL, ey, lQ, ae, eu, BN, cH, r, iy, eK, cl, ac, hc, dh, cj, uo, at, ut, lS, ei, lC, ne); #c1(EPHX3) c2(NP_001136358) c3(epoxide hydrolase 3 precursor) c4(54859) c5(54860, 54861, 54862, 54863, 54864, 54865, 54866, 54867, 54868, 54869, 54870, 54871, 54872, 54873) c6(54874, 54875, 54876, 54877) c7(Kd, T, dt, hl, Ke, aA, n, Vd, Kh, jX, PJ, ci, fr, eA, ad, ip, aaF, jz, hb, aT, bK, K, bU, ae, aW, Y, yx, as, bz, op, dS, bF, Ew, jV, c, iA, vA, U, B); #c1(EPM2A) c2(NP_001018051) c3(laforin isoform b) c4(26944, 26945) c5(26946, 26947, 26948, 26949, 26950, 26951, 26952, 26953, 26954, 26955, 26956, 26957) c6( )c7(T, yr, d, ajm, ha, U, jh, yT, yZ, yX, K, yA, yU, xg, xt); #c1(EPM2A) c2(NP_005661) c3(laforin isoform a) c4(158354, 158355) c5(158356, 158357, 158358, 158359, 158360, 158361, 158362, 158363, 158364, 158365, 158366, 158367) c6( )c7(T, yr, d, ajm, ha, U, jh, yT, yZ, yX, K, yA, yU, xg, xt); #c1(EPM2A) c2(XP_011534416) c3 (laforin isoform X2) c4(330921, 330922) c5(330923, 330924, 330925, 330926, 330927, 330928, 330929, 330930, 330931, 330932, 330933, 330934) c6( )c7(T, yr, d, ajm, ha, U, jh, yT, yZ, yX, K, yA, yU, xg, xt); #c1(EPMZAIP1) c2(NP_055620) c3(EPMZA-interacting protein 1) c4(182315) c5(182316, 182317, 182318, 182319, 182320, 182321) c6( )c7(yU, yT, v, yZ, aT); #c1(EPN1) c2(NP_001123543) c3(epsin-1 isoform a) c4(47467) c5(47468, 47469, 47470, 47471, 47472, 47473, 47474) c6( )c7(T); #c1(EPN1) c2(NP_037465) c3(epsin-1 isoform c) c4(175642) c5(175643, 175644, 175645, 175646, 175647, 175648, 175649) c6( )c7(T); #c1(EPN1) c2(XP_005258886) c3(epsin-1 isoform X1) c4(275851) c5(275852, 275853, 275854, 275855, 275856, 275857) c6( )c7(T); #c1(EPN1) c2(XP_005258887) c3(epsin-1 isoform X2) c4(275858) c5(275859, 275860, 275861, 275862, 275863, 275864) c6( )c7(T); #c1(EPO) c2(NP_000790) c3(erythropoietin precursor) c4(15789) c5(15790, 15791, 15792, 15793, 15794, 15795, 15796, 15797, 15798, 15799, 15800, 15801, 15802, 15803, 15804, 15805, 15806, 15807, 15808, 15809, 15810, 15811, 15812, 15813, 15814, 15815, 15816, 15817, 15818, 15819, 15820, 15821, 15822, 15823, 15824, 15825, 15826, 15827, 15828, 15829, 15830, 15831, 15832, 15833, 15834, 15835, 15836, 15837, 15838, 15839, 15840, 15841, 15842, 15843, 15844, 15845, 15846, 15847, 15848, 15849, 15850, 15851, 15852, 15853, 15854, 15855, 15856, 15857, 15858, 15859, 15860, 15861, 15862, 15863, 15864, 15865, 15866, 15867, 15868, 15869, 15870, 15871, 15872, 15873, 15874, 15875, 15876, 15877, 15878, 15879, 15880, 15881, 15882, 15883, 15884, 15885, 15886, 15887, 15888, 15889, 15890, 15891, 15892, 15893, 15894, 15895, 15896, 15897, 15898, 15899, 15900, 15901, 15902, 15903, 15904, 15905, 15906, 15907, 15908, 15909, 15910, 15911, 15912, 15913, 15914, 15915, 15916, 15917, 15918, 15919, 15920, 15921, 15922, 15923, 15924, 15925, 15926, 15927, 15928, 15929, 15930, 15931, 15932, 15933, 15934, 15935, 15936, 15937, 15938, 15939, 15940, 15941, 15942, 15943, 15944, 15945, 15946, 15947, 15948, 15949, 15950, 15951, 15952, 15953, 15954, 15955, 15956, 15957, 15958, 15959, 15960, 15961, 15962, 15963, 15964, 15965, 15966, 15967, 15968, 15969, 15970, 15971, 15972, 15973, 15974, 15975, 15976, 15977, 15978, 15979, 15980, 15981, 15982, 15983, 15984, 15985, 15986, 15987, 15988, 15989, 15990, 15991, 15992, 15993, 15994, 15995, 15996, 15997, 15998, 15999, 16000, 16001, 16002, 16003, 16004, 16005, 16006, 16007, 16008, 16009, 16010, 16011, 16012, 16013, 16014, 16015, 16016, 16017, 16018, 16019, 16020, 16021, 16022, 16023, 16024, 16025, 16026, 16027, 16028, 16029, 16030, 16031, 16032, 16033, 16034, 16035, 16036, 16037, 16038, 16039, 16040, 16041, 16042, 16043, 16044, 16045, 16046, 16047, 16048, 16049, 16050, 16051, 16052, 16053, 16054, 16055, 16056, 16057, 16058, 16059, 16060, 16061, 16062, 16063, 16064, 16065, 16066, 16067) c6(16068, 16069, 16070, 16071, 16072) c7(T, if, e, qx, hE, fd, Vd, ci, aY, aR, ip, bmL, x, wM, fm, aeO, M, dV, K, hm, LQ, CG, aW, qS, fc, aoD, eE, cm, bw, bml, wT, eM, aNv, nB, iA, vA, wB, U, cj, vy, QF, dW, nw, mk, k, m, R, pw, lS, CI, qm, Ro, aZt, cZ, aw, n, aj, fr, qN, aCQ, bkU, eh, cd, br, eW, ke, Mo, jz, cc, pe, BR, bmN, ql, h, lG, eV, DU, dd, vx, cY, OV, A, fe, aq, kp, pm, hq, iO, ok, GU, bmP, dB, xO, kz, eD, hL, at, E, pg, ER, cB, B, bL, lk, dA, eR, kA, jx, eX, cz, wo, GC, hy, v, j, V, q, J, eA, eJ, oT, RI, yw, hb, HC, aT, yu, cy, dz, ae, eu, sb, Uf, il, oV, Y, atl, r, en, t, ik, ck, bmS, BU, rR, ac, c, pZ, bal, bmR, fP, mf, dy, Mn, oX, bt, VB, ao, vi, hS, bmQ, ob, qb, acl, bs, bmJ, oZ, fu, tK, vN, ad, bmK, dP, fU, iC, CJ, F, ib, alp, py, yt, oz, arV, lC, oW, iY, bmM, d, lR, et, ff, kD, yy, VI, S, avG, aot, iR, dr, ek, bF, es, D, alm, age, qe, pc, bmO, gZ, aS, zk, vt, xg); #c1(EPOR) c2(NP_000112) c3(erythropoietin receptor precursor) c4(1877) c5(1878, 1879, 1880, 1881, 1882, 1883) c6( )c7(G, T, eR, e, cz, fd, hy, j, ci, fr, bkU, eA, ad, bml, md, dP, oT, iC, ib, jz, x, yt, HC, aT, oW, cy, dz, K, hS, ae, af, cY, jf, je, aW, Y, ff, en, avG, g, t, cm, pm, bw, ck, es, il, YE, pc, eM, nB, dB, iA, iM, hl, pg, m, k, iu, VB, Ro); #c1(EPPIN) c2(NP_001289790) c3 (eppin isoform 2 precursor) c4(114617) c5(114618, 114619, 114620, 114621, 114622, 114623, 114624) c6(114625) c7(gU); #c1(EPPIN) c2(NP_065131) c3(eppin isoform 1 precursor) c4(204092, 204093) c5(204094, 204095, 204096, 204097, 204098, 204099, 204100, 204101, 204102, 204103, 204104, 204105, 204106, 204107, 204108, 204109) c6(204110, 204/11, 204112, 204113) c7(gU); #c1(EPPIN-WFDC6) c2(NP_001185915) c3(SPINLW1-WFDC6 fusion protein precursor) c4(76738) c5(76739, 76740, 76741, 76742, 76743, 76744, 76745, 76746, 76747, 76748, 76749, 76750, 76751, 76752) c6(76753, 76754, 76755, 76756) c7(gU); #c1(EPPK1) c2(XP_011515627) c3(epiplakin isoform X1) c4(308462, 308463) c5(308464, 308465, 308466, 308467, 308468, 308469, 308470, 308471, 308472, 308473, 308474, 308475) c6( )c7(NI, aYD); #c1(EPRS) c2(NP_004437) c3(bifunctional glutamate/proline-tRNA ligase) c4(146318, 146319, 146320) c5(146321, 146322, 146323, 146324, 146325, 146326, 146327, 146328, 146329, 146330, 146331, 146332, 146333, 146334, 146335, 146336, 146337, 146338) c6( )c7(dD, ea, e, ha, aw, gZ, cy, j, eq, QK); #c1(EPS15L1) c2(NP_001245303) c3(epidermal growth factor receptor substrate 15-like 1 isoform 1) c4(93050) c5(93051, 93052, 93053, 93054, 93055, 93056) c6( )c7(V, B); #c1(EPS15LI) c2(NP_001245304) c3(epidermal growth factor receptor substrate 15-like 1 isoform 3) c4(93057) c5(93058, 93059, 93060, 93061, 93062, 93063) c6( ) c7(V, B); #c1(EPS15L1) c2(NP_001245305) c3(epidermal growth factor receptor substrate 15-like 1 isoform 4) c4(93064) c5(93065, 93066, 93067, 93068, 93069, 93070) c6( )c7(V, B); #c1(EPS15LI) c2(NP_067058) c3(epidermal growth factor receptor substrate 15-like 1 isoform 2) c4(208774) c5(208775, 208776, 208777, 208778, 208779, 208780) c6( )c7(V, B); #c1(EPS8) c2(XP_005253396) c3(epidermal growth factor receptor kinase substrate 8 isoform X3) c4(272454, 272455, 272456) c5(272457, 272458, 272459, 272460, 272461, 272462, 272463) c6( )c7(T, jz, d, cF, yt, aW, ff, aT, hR, ci, m, nA, en, t, bN); #c1(EPS8) c2(XP_011518907) c3(epidermal growth factor receptor kinase substrate 8 isoform X2) c4(313284) c5(313285, 313286) c6( )c7(T, jz, d, cF, yt, aW, ff, aT, hR, ci, m, nA, en, t, bN); #c1(EPS8) c2(XP_011518908) c3(epidermal growth factor receptor kinase substrate 8 isoform X1) c4(313287, 313288, 313289) c5(313290, 313291, 313292, 313293, 313294, 313295, 313296, 313297, 313298, 313299, 313300, 313301, 313302, 313303, 313304, 313305, 313306, 313307, 313308, 313309, 313310, 313311, 313312, 313313, 313314, 313315, 313316, 313317, 313318, 313319, 313320, 313321, 313322, 313323, 313324, 313325, 313326, 313327, 313328, 313329, 313330, 313331, 313332, 313333, 313334, 313335, 313336, 313337, 313338) c6( )c7(T, jz, d, cF, yt, aW, ff, aT, hR, ci, m, nA, en, t, bN); #c1(EPS8L2) c2(NP_073609) c3(epidermal growth factor receptor kinase substrate 8-like protein 2) c4(213074, 213075, 213076, 213077, 213078, 213079) c5(213080, 213081, 213082, 213083, 213084, 213085, 213086, 213087, 213088, 213089, 213090, 213091, 213092, 213093, 213094, 213095, 213096, 213097, 213098, 213099, 213100, 213101, 213102, 213103, 213104, 213105, 213106, 213107, 213108, 213109, 213110, 213111, 213112, 213113, 213114, 213115, 213116, 213117, 213118, 213119, 213120, 213121, 213122, 213123) c6( )c7(fe); #c1(EPSTI1) c2(NP_001002264) c3(epithelial-stromal interaction protein 1 isoform 1) c4(19505) c5(19506, 19507, 19508, 19509, 19510, 19511) c6( )c7(aW, T, aT); #c1(EPSTI1) c2(NP_150280) c3(epithelial-stromal interaction protein 1 isoform 2) c4(228022) c5(228023, 228024, 228025, 228026, 228027) c6( )c7(aW, T, aT); #c1(EPSTI1) c2(XP_005266653) c3(epithelial-stromal interaction protein 1 isoform X1) c4( )c5(280272) c6( )c7(aW, T, aT); #c1(EPT1) c2(NP_277040) c3 (ethanolaminephosphotransferase 1) c4(228779, 228780) c5(228781, 228782, 228783, 228784, 228785, 228786, 228787, 228788, 228789, 228790, 228791, 228792) c6( )c7(qb, i0); #c1(EPX) c2(NP_000493) c3(eosinophil peroxidase preproprotein) c4(9916) c5(9917, 9918, 9919, 9920, 9921, 9922) c6( )c7(T, eR, NT, bB, fr, bkU, q, ad, eA, Ro, x, oT, alp, fm, oW, cy, ae, d, lR, r, aq, pm, bw, ck, alm, bmU, c, dB, bE, eM, mf, k, Mn, B, bmT); #c1(EPYC) c2(NP_004941) c3 (epiphycan precursor) c4(151310, 151311) c5(151312, 151313, 151314, 151315, 151316, 151317, 151318, 151319, 151320, 151321, 151322, 151323, 151324, 151325, 151326, 151327) c6(151328, 151329, 151330, 151331) c7(bmV, asg, Be); #c1(ERAL1) c2(NP_005693) c3(GTPase Era, mitochondrial) c4(158604, 158605) c5(158606, 158607, 158608, 158609, 158610, 158611) c6( )c7(ai, aW, eZ, aT, cl, dy, cd, o); #c1(ERAP1) c2(NP_001185470) c3(endoplasmic reticulum aminopeptidase 1 isoform b precursor) c4(75674) c5(75675, 75676, 75677, 75678, 75679, 75680, 75681, 75682, 75683, 75684, 75685, 75686, 75687, 75688, 75689, 75690) c6( )c7(T, jv, ex, bmW, cz, cl, qN, V, ai, q, o, wD, h, eV, hH, A, mR, cr, fe, lo, cl, uC, qa, Jr, AM, Tr); #c1(ERAP1) c2(NP_057526) c3(endoplasmic reticulum aminopeptidase 1 isoform a precursor) c4(191253) c5(191254, 191255, 191256, 191257, 191258, 191259) c6( )c7(T, jv, ex, bmW, cz, cl, qN, V, ai, q, o, wD, h, eV, hH, A, mR, cr, fe, lo, cl, uC, qa, Jr, AM, Tr); #c1(ERAP1) c2(XP_011541787) c3(endoplasmic reticulum aminopeptidase 1 isoform X3) c4(340382) c5( )c6( )c7(T, jv, ex, bmW, cz, cl, qN, V, ai, q, o, wD, h, eV, hH, A, mR, cr, fe, lo, cl, uC, qa, Jr, AM, Tr); #c1(ERAP2) c2(NP_001123612) c3(endoplasmic reticulum aminopeptidase 2) c4(47794, 47795, 47796) c5(47797, 47798, 47799, 47800, 47801, 47802, 47803, 47804, 47805, 47806, 47807, 47808, 47809, 47810, 47811, 47812, 47813, 47814) c6( )c7(jv, mR, A, cz, h, lo, cm, uC, qa); #c1(ERAP2) c2(XP_011541846) c3(endoplasmic reticulum aminopeptidase 2 isoform X1) c4(340401) c5(340402, 340403, 340404, 340405, 340406, 340407) c6( )c7(jv, mR, A, cz, h, lo, cm, uC, qa); #c1(ERAS) c2(NP_853510) c3(GTPase ERas precursor) c4(255135) c5(255136, 255137, 255138, 255139, 255140, 255141) c6( )c7(av, Vy, ht, ci); #c1(ERBB2) c2(NP_001005862) c3(receptor tyrosine-protein kinase erbB-2 isoform b) c4(22143, 22144) c5( )c6( )c7(gh, T, AY, ci, bv, xy, aE, ajv, auB, hu, cp, li, ZH, iE, Ls, az, nY, zE, iA, nB, wE, bkv, cj, Zn, cX, k, iB, ht, cd, aoc, aex, bkP, Gw, Dz, pl, MD, Lh, CQ, asl, dB, pV, avr, AN, Dh, aA, bX, lu, AO, q, RU, aew, qK, j, aju, NE, eS, cl, bkp, pb, ov, pa, aWQ, aws, bnl, rA, Mb, eP, rQ, iW, jN, jh, OE, Sv, Gn, aue, ad, afb, hX, oW, aln, MG, BO, ff, aeV, bz, i, aed, iu, ne, bmY, bkx, if, bB, ip, bmL, x, Na, ws, Ma, anV, ael, gs, YL, xQ, eG, ajT, JI, Qq, Au, Am, as, ir, eF, cm, Qt, bnp, go, mx, in, afA, lT, n, b, no, jz, bnd, Do, zw, gd, eV, ia, AQ, hQ, Ye, bP, GU, PI, hL, at, nL, B, bmX, AS, dA, eX, v, eJ, asG, BJ, avt, ae, jf, bnk, Or, vT, alr, aDY, apx, t, KQ, ck, aug, bnf, kP, gX, anD, alV, Dd, bnb, hd, bne, db, Ti, YQ, nJ, d, aoW, aWR, anW, gk, bnn, ll, gZ, apv, y, aC, al, alN, lR, AW, gJ, bnj, gH, rJ, pX, Qk, DO, K, eL, LZ, g, ajj, AM, U, PV, m, sw, zX, NT, zP, Nh, ft, By, wb, KV, avc, CN, aGd, cM, Zj, it, aol, fM, avm, fG, j, eA, er, XX, amg, bno, lg, yg, qv, Nc, avg, Y, r, en, ik, bnc, QG, np, EY, LS, GI, id, bnm, iJ, dP, fX, io, hP, Yi, ael, eH, hp, aAr, Ew, Lj, AR, auZ, Gu, hE, ix, DS, G, pf, Fo, bU, z, bnh, aW, eM, wB, Xu, awr, fr, qN, eW, ke, Bl, cc, hw, FN, jy, Nf, dL, hB, asT, afu, aiB, bmZ, aq, kp, mo, op, Qy, Qu, hk, hR, anx, bni, ZZ, adG, cn, sM, tc, hy, bkG, nv, LY, aT, adV, bna, qw, Uf, s, il, oV, aYl, nM, im, ava, Fb, qC, av, Rq, Yo, fb, il, bng, afe, qt, apb, Ej, af, aU, Hp, es, eU, aoR, bE, WK, be, adg, X); #c1(ERBB2) c2(NP_001276865) c3(receptor tyrosine-protein kinase erbB-2 isoform c) c4(108510) c5(108511, 108512, 108513, 108514, 108515, 108516, 108517, 108518, 108519, 108520, 108521, 108522, 108523, 108524, 108525, 108526, 108527, 108528, 108529, 108530) c6( )c7(gh, T, AY, ci, bv, xy, aE, ajv, auB, hu, cp, li, ZH, iE, Ls, az, nY, zE, iA, nB, wE, bkv, cj, Zn, cx, k, iB, ht, cd, aoc, aeX, bkP, Gw, Dz, pl, MD, Lh, CQ, asl, dB, pV, avr, AN, Dh, aA, bX, lu, AO, q, RU, aew, qK, jj, aju, NE, eS, cl, bkp, pb, ov, pa, aWQ, aws, bnl, rA, Mb, eP, rQ, iW, jN, jh, DE, Sv, Gn, que, ad, afb, hX, oW, aln, MG, BO, ff, aeV, bz, i, aed, iu, ne, bmY, bkx, if, bB, ip, bmL, x, Na, ws, Ma, anV, ael, gs, YL, xQ, eG, ajT, JI, Qq, Au, Qm, as, ir, eF, cm, At, bnp, go, mx, in, afA, lT, n, b, no, jz, bnd, Do, zw, gd, eV, ia, Al, hQ, Ye, bP, GU, Pl, hL, at, nL, B, bmX, AS, dA, eX, v, eJ, asG, BJ, avt, ae, jf, bnk, Or, vT, alr, aDY, apx, t, KQ, ck, aug, bnf, kP, gX, anD, alV, Dd, bnb, hd, bne, db, Ti, YQ, nJ, d, aoW, aWR, anW, gk, bnn, ll, gZ, apv, y, aC, al, alN, lR, AW, gJ, bnj, gH, rJ, pX, Qk, DO, K, eL, LZ, g, ajj, AM, U, PV, m, sw, zX, NT, zP, Nh, ft, By, wb, KV, avc, CN, aGd, cM, Zj, it, aoU, fM, avm, fG, j, eA, er, XX, amg, bno, lg, yg, qv, Nc, avg, Y, r, en, iK, bnc, QG, np, EY, LS, Gl, id, bnm, iJ, dP, fX, io, hP, Yi, ael, eH, hp, aAr, Ew, Lj, AR, auZ, Gu, hE, ix, DS, G, pf, Fo, bU, zQ, bnh, aW, eM, wB, Xu, awr, fr, qN, eW, ke, Bl, cc, hw, FN, jy, Nf, dL, hB, asT, afu, aiB, bmZ, aq, kp, mo, op, Qy, Qu, hk, hR, anx, bni, ZZ, adG, cn, sM, tc, hy, bkG, nv, LY, aT, adV, bna, qw, Uf, s, il, oV, aYl, nM, im, ava, Fb, qC, av, Rq, Yo, fb, il, bng, afe, qt, apb, Ej, aF, aU, Hp, es, eU, aoR, bE, WK, be, adg, X); #c1(ERBB2) c2(NP_001276866) c3(receptor tyrosine-protein kinase erbB-2 isoform d precursor) c4( )c5(108531, 108532, 108533, 108534, 108535, 108536) c6( )c7(gh, T, AY, ci, bv, xy, aE, ajv, auB, hu, cp, li, ZH, iE, Ls, az, nY, zE, iA, nB, wE, bkv, cj, Zn, cX, k, iB, ht, cd, aoc, aeX, bkP, Gw, Dz, pl, MD, Lh, CQ, asl, dB, pV, avr, AN, Dh, aA, bX, lu, AD, q, RU, aew, qK, jj, aju, NE, eS, cl, bkp, pb, ov, pa, aWQ, aws, bnl, rA, Mb, eP, rQ, iW, jN, jh, OE, Sv, Gn, aue, ad, afb, hX, oW, aln, MG, BO, ff, aeV, bz, i, aed, iu, ne, bmY, bkx, if, bB, ip, bmL, x, Na, ws, Ma, anV, ael, gs, YL, xQ, eG, ajT, JI, Qq, Au, Qm, as, ir, eF, cm, Qt, bnp, go, mx, in, afA, lT, n, b, no, jz, bnd, Do, zw, gd, eV, ia, AQ, hQ, Ye, bP, GU, PI, hL, at, nL, B, bmX, AS, dA, eX, v, eJ, asG, BJ, avt, ae, jf, bnk, Ar, vT, alr, aDY, apx, t, KQ, ck, aug, bnf, kP, gX, anD, alV, Dd, bnb, hd, bne, db, Ti, YQ, nJ, d, aoW, aWR, anW, gk, bnn, ll, gZ, apv, y, aC, al, alN, lR, AW, gJ, bnj, gH, rJ, pX, Qk, DO, K, eL, LZ, g, ajj, AM, U, PV, m, sw, zX, NT, zP, Nh, ft, By, wb, KV, avc, CN, aGd, cM, Zj, it, aoU, fM, avm, fG, j, eA, er, XX, amg, bno, lg, yg, qv, Nc, avg, Y, r, en, iK, bnc, QG, np, EY, LS, GI, id, bnm, iJ, dP, fX, io, hP, Yi, aeQ, eH, hp, aAr, Ew, Lj, AR, auZ, Gu, hE, ix, DS, G, pf, Fo, bU, zQ, bnh, aW, eM, wB, Xu, awr, fr, qN, eW, ke, BI, cc, hw, FN, jy, Nf, dL, hB, asT, afu, aiB, bmZ, aq, kp, mo, op, By, Qu, hk, hR, anx, bni, ZZ, adG, cn, sM, tc, hy, bkG, nv, LY, aT, adV, bna, qw, Uf, s, iU, oV, aYl, nM, im, ava, Fb, qC, av, Rq, Yo, fb, il, bng, afe, qt, apb, Ej, aF, aUD, Hp, es, eU, aoR, bE, WK, be, adg, X); #c1(ERBB2) c2(NP_004439) c3(receptor tyrosine-protein kinase erbB-2 isoform a precursor) c4(146339, 146340) c5(146341, 146342, 146343, 146344, 146345, 146346, 146347) c6( )c7(gh, T, AY, ci, bv, xy, aE, ajv, auB, hu, cp, li, ZH, iE, Ls, az, nY, zE, iA, nB, wE, bkv, cj, Zn, cX, k, iB, ht, cd, aoc, aeX, bkP, Gw, Dz, pl, MD, Lh, CQ, asl, dB, pV, avr, AN, Dh, aA, bX, lu, AO, q, RU, aew, qK, jj, aju, NE, eS, cl, bkp, pb, ov, pa, aWQ, aws, bnl, rA, Mb, eP, rQ, iW, jN, jh, OE, Sv, Gn, aue, ad, afb, hX, oW, aln, MG, BO, ff, aeV, bz, i, aed, iu, ne, bmY, bkx, if, bB, ip, bmL, x, Na, ws, Ma, anV, aeO, gs, YL, xl, eG, ajT, JI, Qq, Au, Am, as, ir, eF, cm, Qt, bnp, go, mx, in, afA, lT, n, b, no, jz, bnd, Do, zw, gd, eV, ia, AQ, hQ, Ye, bP, GU, Pl, hL, at, nL, B, bmX, AS, dA, eX, v, eJ, asG, BJ, avt, ae, jf, bnk, Or, vT, alr, aDY, apx, t, KQ, ck, aug, bnf, kP, gX, anD, alV, Dd, bnb, hd, bne, db, Ti, YQ, nJ, d, aoW, aWR, anW, gk, bnn, ll, gZ, apv, y, aC, al, alN, lR, AW, gJ, bnj, gH, rJ, pX, Qk, DO, K, eL, LZ, g, ajj, AM, U, PV, m, sw, zX, NT, zP, Nh, ft, By, wb, KV, avc, CN, aGd, cM, Zj, it, aol, fM, avm, fG, j, eA, er, XX, amg, bno, lg, yg, qv, Nc, avg, Y, r, en, iK, bnc, QG, np, EY, LS, Gl, id, bnm, iJ, dP, fX, io, hP, Yi, aeQ, eH, hp, aAr, Ew, Lj, AR, auZ, Gu, HE, ix, DS, G, pf, Fo, bU, zQ, bnh, aW, eM, wB, Xu, awr, fr, qN, eW, ke, BI, cc, hw, FN, jy, Nf, dL, hB, asT, afu, aiB, bmZ, aq, kp, mo, op, Qy, Qu, hk, hR, anx, bni, ZZ, adG, cn, sM, tc, hy, bkG, nv, LY, aT, adV, bna, qw, Uf, s, iU, oV, aYl, nM, im, ava, Fb, qC, av, Rq, Yo, fb, il, bng, afe, qt, apb, Ej, aF, aUO, Hp, es, eU, aoR, bE, WK, be, adg, X); #c1(ERBB2IP) c2(NP_001006600) c3(protein LAP2 isoform 7) c4(22188) c5(22189, 22190, 22191, 22192, 22193, 22194, 22195, 22196, 22197, 22198) c6( ) c7(nZ, T, jz, eW, qK, mx, asL, fu, XM, bc); #c1(ERBB2IP) c2(NP_001240626) c3(protein LAP2 isoform 1) c4(88624) c5(88625, 88626, 88627, 88628, 88629, 88630) c6( )c7(nZ, T, jz, eW, qK, mx, asL, fu, XM, bc); #c1(ERBB2IP) c2(NP_001240627) c3(protein LAP2 isoform 4) c4(88631) c5(88632, 88633, 88634, 88635, 88636, 88637) c6( )c7(nZ, T, jz, eW, qK, mx, asl, fu, XM, bc); #c1(ERBB2IP) c2(NP_001240628) c3(protein LAP2 isoform 8) c4(88638, 88639, 88640) c5(88641, 88642, 88643, 88644, 88645, 88646) c6( ) c7(nZ, T, jz, eW, qK, mx, asl, fu, XM, bc); #c1(ERBB2IP) c2(NP_061165) c3(protein LAP2 isoform 2) c4(199674) c5(199675, 199676, 199677, 199678, 199679, 199680, 199681, 199682, 199683, 199684, 199685, 199686, 199687, 199688, 199689, 199690, 199691, 199692) c6( )c7(nZ, T, jz, eW, qK, mx, asL, fu, XM, bc); #c1(ERBB3) c2(NP_001005915) c3(receptor tyrosine-protein kinase erbB-3 isoform s precursor) c4(22154) c5(22155, 22156, 22157, 22158, 22159, 22160, 22161, 22162, 22163, 22164, 22165, 22166) c6( )c7(T, Tb, pf, fd, bB, cL, ci, ip, yM, bv, aE, anV, Ok, bnt, K, bU, hu, De, ZH, aW, Au, g, as, eF, cm, Qt, eM, nB, go, m, Xu, in, lT, lo, yG, NT, fr, qN, aoc, dL, hB, A, CN, AQ, kp, op, GU, dB, Qu, bns, at, bL, fG, aA, cz, j, AD, V, q, eA, eJ, aT, hK, ae, s, oV, Y, bnq, r, en, cl, t, ik, np, im, LS, id, ad, av, Tg, nJ, Yi, bnr, yF, d, ff, eH, eZ, aF, hp, adj, bz, Hp, bE, X, Ug, y, iu); #c1(ERBB3) c2(NP_001973) c3(receptor tyrosine-protein kinase erbB-3 isoform 1 precursor) c4(122753, 122754, 122755, 122756, 122757, 122758) c5(122759, 122760, 122761, 122762, 122763, 122764, 122765, 122766, 122767, 122768, 122769, 122770, 122771, 122772, 122773, 122774, 122775, 122776, 122777, 122778, 122779, 122780, 122781, 122782, 122783, 122784, 122785, 122786, 122787, 122788) c6( )c7(T, Tb, pf, fd, bB, cL, ci, ip, yM, bv, aE, anV, Qk, bnt, K, bU, hu, De, ZH, aW, Au, g, as, eF, cm, Qt, eM, nB, go, m, Xu, in, lT, lo, yG, NT, fr, qN, aoc, dL, hB, A, CN, AQ, kp, op, GU, dB, Qu, bns, at, bL, fG, aA, cz, j, AO, V, q, eA, eJ, aT, hK, ae, s, oV, Y, bnq, r, en, cl, t, ik, np, im, LS, id, ad, av, Tg, nJ, Yi, bnr, yF, d, ff, eH, eZ, aF, hp, adj, bz, Hp, bE, X, Ug, y, iu); #c1(ERBB4) c2(NP_001036064) c3(receptor tyrosine-protein kinase erbB-4 isoform JM-a/CVT-2 precursor) c4(33899) c5(33900, 33901, 33902, 33903, 33904, 33905, 33906) c6( )c7(T, aC, if, bB, ci, aE, K, hu, eL, aW, Ls, g, eF, cm, mZ, eM, iA, go, m, lT, ht, n, fr, qN, cd, aoc, dL, hB, mY, mV, he, dx, bnv, mU, MD, fe, cM, mX, GU, dB, hk, aur, it, mT, bL, mS, ti, v, q, eA, aT, bnu, bK, j, rz, af, jf, hM, Y, r, en, t, ik, amd, ac, c, id, ha, ad, XB, av, py, mW, io, bkw, ji, nJ, Yi, d, Gv, ff, eH, aF, bz, Hp, es, D, bE, dO, kS); #c1(ERBB4) c2(NP_005226) c3(receptor tyrosine-protein kinase erbB-4 isoform JM-a/CVT-I precursor) c4(154222) c5(154223, 154224, 154225, 154226, 154227, 154228, 154229) c6( ) c7(T, aC, if, bB, ci, aE, K, hu, eL, aW, Ls, g, eF, cm, mZ, eM, iA, go, m, lT, ht, n, fr, qN, cd, aoc, dL, hB, mY, mV, he, dx, bnv, mU, MD, fe, cM, mx, GU, dB, hk, aur, it, mT, bL, mS, ti, v, q, eA, aT, bnu, bK, j, rz, af, jf, hM, Y, r, en, t, ik, amd, ac, c, id, ha, ad, XB, av, py, mW, io, bkw, ji, nJ, Yi, d, Gv, ff, eH, aF, bz, Hp, es, D, bE, dO, kS); #c1(ERBB4) c2(XP_005246433) c3(receptor tyrosine-protein kinase erbB-4 isoform X3) c4(267112, 267113) c5(267114, 267115, 267116, 267117, 267118, 267119) c6( )c7(T, aC, if, bB, ci, aE, K, hu, eL, aW, Ls, g, eF, cm, mZ, eM, iA, go, m, lT, ht, n, fr, qN, cd, aoc, dL, hB, mY, mV, he, dx, bnv, ml, MD, fe, cM, mX, GU, dB, hk, aur, it, mT, bL, mS, ti, v, q, eA, aT, bnu, bK, jj, rz, af, jf, hM, Y, r, en, t, ik, amd, ac, c, id, ha, ad, XB, av, py, mW, io, bkw, ji, nJ, Yi, d, Gv, ff, eH, aF, bz, Hp, es, D, bE, dO, kS); #c1(ERBB4) c2(XP_005246434) c3(receptor tyrosine-protein kinase erbB-4 isoform X4) c4( )c5(267120, 267121, 267122, 267123, 267124, 267125, 267126, 267127, 267128, 267129, 267130, 267131) c6( )c7(T, aC, if, bB, ci, aE, K, hu, eL, aW, Ls, g, ef, cm, mZ, eM, iA, go, m, lT, ht, n, fr, qN, cd, aoc, dL, hB, mY, mV, he, dx, bnv, mU, MD, fe, cM, mX, GU, dB, hk, aur, it, mT, bL, mS, ti, v, q, eA, aT, bnu, bK, jj, rz, af, jf, hM, Y, r, en, t, iK, amd, ac, c, id, ha, ad, XB, av, py, mW, io, bkw, ji, nJ, Yi, d, Gv, ff, eH, aF, bz, Hp, es, D, bE, dO, kS); #c1(ERBB4) c2(XP_006712427) c3(receptor tyrosine-protein kinase erbB-4 isoform X1) c4(286648) c5( )c6( )c7(T, aC, if, bB, ci, aE, K, hu, eL, aW, Ls, g, ef, cm, mZ, eM, iA, go, m, lT, ht, n, fr, qN, cd, aoc, dL, hB, mY, mV, he, dx, bnv, mU, MD, fe, cM, mX, GU, dB, hk, aur, it, mT, bL, mS, ti, v, q, eA, aT, bnu, bK, jj, rz, af, jf, hM, Y, r, en, t, ik, amd, ac, c, id, ha, ad, XB, av, py, mW, io, bkw, ji, nJ, Yi, d, Gv, ff, eH, aF, bz, Hp, es, D, bE, do, kS); #c1(ERC1) c2(XP_011519238) c3(ELKS/Rab6-interacting/CAST family member 1 isoform X3) c4(313828, 313829) c5(313830, 313831, 313832, 313833, 313834, 313835, 313836, 313837, 313838, 313839, 313840, 313841) c6( )c7(hd, nM, nB, n, bt, fq, anU); #c1(ERC1) c2(XP_011519239) c3(ELKS/Rab6-interacting/CAST family member 1 isoform X4) c4(313842) c5( )c6( )c7(hd, nM, nB, n, bt, fq, anl); #c1(ERC1) c2(XP_011519240) c3(ELKS/Rab6-interacting/CAST family member 1 isoform X5) c4(313843) c5( )c6( )c7(hd, nM, nB, n, bt, fq, anl); #c1(ERC1) c2(XP_011519241) c3(ELKS/Rab6-interacting/CAST family member 1 isoform X6) c4( )c5(313844, 313845, 313846, 313847, 313848, 313849, 313850, 313851, 313852, 313853, 313854, 313855) c6( )c7(hd, nM, nB, n, bt, fq, anU); #c1(ERC1) c2(XP_011519243) c3(ELKS/Rab6-interacting/CAST family member 1 isoform X1) c4(313856) c5(313857, 313858, 313859, 313860, 313861, 313862, 313863, 313864, 313865, 313866, 313867, 313868, 313869, 313870, 313871, 313872, 313873, 313874) c6( )c7(hd, nM, nB, n, bt, fq, anl); #c1(ERC1) c2(XP_011519244) c3(ELKS/Rab6-interacting/CAST family member 1 isoform X2) c4(313875) c5(313876, 313877, 313878, 313879, 313880, 313881) c6( )c7(hd, nM, nB, n, bt, fq, anl); #c1(ERC1) c2(XP_011519246) c3(ELKS/Rab6-interacting/CAST family member 1 isoform X9) c4(313882, 313883, 313884) c5(313885, 313886, 313887, 313888, 313889, 313890, 313891, 313892, 313893, 313894, 313895, 313896, 313897, 313898, 313899, 313900, 313901, 313902) c6( )c7(hd, nM, nB, n, bt, fq, anU); #c1(ERC2) c2(NP_056391) c3(ERC protein 2) c4(187399) c5(187400, 187401, 187402, 187403, 187404, 187405, 187406, 187407, 187408) c6( )c7(Kd, dD, VJ, akX, ea, Fm, Y, aw, wB, jh, cz, n, eq, fr, ji, an, he, cd, Hr); #c1(ERC2) c2(XP_011531882) c3(ERC protein 2 isoform X1) c4(327932) c5(327933, 327934, 327935, 327936, 327937, 327938) c6( )c7(Kd, dD, VJ, akX, ea, Fm, Y, aw, wB, jh, cz, n, eq, fr, ji, an, he, cd, Hr); #c1(ERC2) c2(XP_011531883) c3(ERC protein 2 isoform X2) c4( )c5(327939) c6( )c7(Kd, dD, VJ, akX, ea, Fm, Y, aw, wB, jh, cz, n, eq, fr, ji, an, he, cd, Hr); #c1 (ERCC1) c2(NP_001159521) c3(DNA excision repair protein ERCC-1 isoform 3) c4(64191) c5(64192, 64193, 64194, 64195, 64196, 64197, 64198, 64199, 64200, 64201, 64202) c6( )c7(T, os, aC, pf, if, bB, ci, x, gJ, bkA, aE, ja, K, hu, eL, bny, JI, aW, CT, nY, La, NR, g, lm, eF, bw, eM, iT, vy, cj, PV, k, m, iu, gU, bnw, ht, qy, qN, br, ft, rC, gd, lG, Gw, dL, hT, PX, hQ, bJ, dB, hL, at, it, B, bL, fM, yk, aA, hy, bX, Qo, V, q, eA, yg, aT, vH, bnx, ahE, ae, ne, wr, ND, Y, Lf, apk, r, ahl, en, bu, cl, t, iK, ck, sS, BU, gX, dy, rA, bt, hS, gV, GI, Dd, iW, oZ, oH, Gn, ad, av, vJ, NS, d, hG, ff, aF, bz, dR, bE, Lb, bC, Gu, Pk, X); #c1(ERCC1) c2(NP_001974) c3(DNA excision repair protein ERCC-1 isoform 2) c4(122789, 122790) c5(122791, 122792, 122793, 122794, 122795, 122796, 122797, 122798, 122799, 122800) c6( )c7(T, os, aC, pf, if, bB, ci, x, gJ, bkA, aE, ja, K, hu, eL, bny, JI, aW, CT, nY, La, NR, g, lm, eF, bw, eM, iT, vy, cj, PV, k, m, iu, gU, bnw, ht, qy, qN, br, ft, rC, gd, lG, Gw, dL, hT, PX, hQ, bJ, dB, hL, at, it, B, bL, fM, yk, aA, hy, bX, Do, V, q, eA, yg, aT, vH, bnx, ahE, ae, ne, wr, ND, Y, Lf, apK, r, ahl, en, bu, cl, t, ik, ck, sS, BU, gX, dy, rA, bt, hS, gV, GI, Dd, iW, oZ, oH, Gn, ad, av, vJ, NS, d, hG, ff, aF, bz, dR, bE, Lb, bC, Gu, Pk, X); #c1(ERCC1) c2(XP_005258693) c3(DNA excision repair protein ERCC-1 isoform X1) c4(275707, 275708) c5(275709, 275710, 275711, 275712, 275713, 275714, 275715, 275716) c6(275717, 275718) c7(T, os, aC, pf, if, bB, ci, x, gJ, bkA, aE, ja, K, hu, eL, bny, JI, aW, CT, nY, La, NR, g, lm, eF, bw, eM, iT, vy, cj, PV, k, m, iu, gU, bnw, ht, qy, qN, br, ft, rC, gd, lG, Gw, dL, hT, PX, hQ, bJ, dB, hL, at, it, B, bL, fM, yk, aA, hy, bX, Do, V, q, eA, yg, aT, vH, bnx, ahE, ae, ne, wr, ND, Y, Lf, apk, r, ahl, en, bu, cl, t, iK, ck, sS, BU, gX, dy, rA, bt, hS, gV, GI, Dd, iW, oZ, oH, Gn, ad, av, vJ, NS, d, hG, ff, aF, bz, dR, bE, Lb, bC, Gu, Pk, X); #c1(ERCC2) c2(NP_000391) c3(TFIIH basal transcription factor complex helicase XPD subunit isoform 1) c4(8409, 8410, 8411) c5(8412, 8413, 8414, 8415, 8416, 8417, 8418, 8419, 8420, 8421, 8422, 8423, 8424, 8425, 8426, 8427, 8428, 8429) c6( )c7(atT, os, T, aC, pf, if, fd, axk, ci, ip, x, aE, Qk, K, bU, hu, pk, eL, zQ, aW, CT, LZ, nY, kt, La, g, eF, zE, lk, uC, bw, ou, fT, eM, nB, iT, cj, k, m, cE, Vj, bnA, gU, ht, NT, n, fr, nA, qN, ke, ft, no, hw, aXH, rC, gd, eV, dL, hB, ny, AQ, hQ, aq, pm, cM, op, W, dB, hk, hL, at, uz, NB, it, B, cn, bnz, fM, yk, jx, ex, aA, hy, j, bX, Do, V, q, eA, MR, hW, afQ, yg, aT, vH, ae, qw, s, aBq, Y, Lf, r, en, bu, cl, t, iK, ck, sS, kP, Dy, ac, pZ, bt, rA, hS, gV, GI, id, iW, jh, oZ, Gn, fu, hF, Fc, iJ, ad, hX, dP, av, bnC, hd, ib, NS, nl, io, lQ, BN, hG, ff, eH, nh, eZ, CF, aF, bz, dr, es, pc, bE, lM, bnB, iu, rk, BA); #c1 (ERCC2) c2(NP_001124339) c3(TFIIH basal transcription factor complex helicase XPD subunit isoform 2) c4(48734) c5(48735, 48736, 48737, 48738, 48739, 48740) c6( )c7(atT, os, T, aC, pf, if, fd, axk, ci, ip, x, aE, Qk, K, bU, hu, pk, eL, zQ, aW, CT, LZ, nY, kt, La, g, eF, zE, lk, uC, bw, ou, fT, eM, nB, iT, cj, k, m, cE, Vj, bnA, gU, ht, NT, n, fr, nA, qN, ke, ft, no, hw, aXH, rC, gd, eV, dL, hB, ny, AQ, hl, aq, pm, cM, op, W, dB, hk, hL, at, uz, NB, it, B, cn, bnz, fM, yk, jx, eX, aA, hy, j, bX, Qo, V, q, eA, MR, hW, afQ, yg, aT, vH, ae, qw, s, aBq, Y, Lf, r, en, bu, cl, t, iK, ck, sS, kP, Dy, ac, pZ, bt, rA, hS, gV, Gl, id, iW, jh, oZ, Gn, fu, hF, Fc, iJ, ad, hX, dP, av, bnC, hd, ib, NS, nl, io, lQ, BN, hG, ff, eH, nh, eZ, CF, af, bz, dr, es, pc, bE, lM, bnB, iu, rk, BA); #c1(ERCC2) c2(XP_011524913) c3(TFIIH basal transcription factor complex helicase XPD subunit isoform X1) c4(319307, 319308) c5(319309, 319310, 319311, 319312, 319313, 319314, 319315, 319316, 319317, 319318, 319319, 319320) c6( ) c7(atT, os, T, aC, pf, if, fd, axk, ci, ip, x, aE, Qk, K, bU, hu, pk, eL, zQ, aW, CT, LZ, nY, kt, La, g, eF, zE, lk, uC, bw, ou, fT, eM, nB, iT, cj, k, m, cE, Vj, bnA, gU, ht, NT, n, fr, nA, qN, ke, ft, no, hw, aXH, rC, gd, eV, dL, hB, ny, Al, hl, aq, pm, cM, op, W, dB, hk, hl, at, uz, NB, it, B, cn, bnz, fM, yk, jx, eX, aA, hy, j, bX, Qo, V, q, eA, MR, hW, af Q, yg, aT, vH, ae, qw, s, aBq, Y, Lf, r, en, bu, cl, t, ik, ck, sS, kP, Dy, ac, pZ, bt, rA, hS, gV, Gl, id, iW, jh, oZ, Gn, fu, hF, Fc, iJ, ad, hX, dP, av, bnC, hd, ib, NS, nl, io, lQ, BN, hG, ff, eH, nh, eZ, CF, aF, bz, dr, es, pc, bE, lM, bnB, iu, rk, BA); #c1(ERCC3) c2(NP_000113) c3(TFIIH basal transcription factor complex helicase XPB subunit isoform a) c4(1884) c5(1885, 1886, 1887, 1888, 1889, 1890, 1891, 1892, 1893, 1894, 1895, 1896, 1897) c6( )c7(T, yk, iW, ht, bX, V, eA, ad, ke, av, no, MR, aT, dL, K, lQ, ae, hG, aW, bnD, Y, aBq, CT, r, La, t, ik, bz, dr, at, m, gn, Pk, bnB, B, bnA); #c1(ERCC3) c2(NP_001290347) c3(TFIIH basal transcription factor complex helicase XPB subunit isoform b) c4(115220) c5( )c6( )c7(T, yk, iW, ht, bX, V, eA, ad, ke, av, no, MR, aT, dL, K, lQ, ae, hG, aW, bnD, Y, aBq, CT, r, La, t, ik, bz, dr, at, m, gn, Pk, bnB, B, bnA); #c1(ERCC4) c2(NP_005227) c3(DNA repair endonuclease XPF) c4(154230, 154231, 154232) c5(154233, 154234, 154235, 154236, 154237, 154238, 154239, 154240, 154241, 154242, 154243, 154244, 154245, 154246, 154247, 154248, 154249, 154250) c6( )c7(bL, Gl, T, os, gU, aC, bnw, ht, aA, n, aj, hy, Gn, oZ, bX, V, q, J, ad, eA, eJ, ip, av, gJ, aE, Qk, rC, yg, aT, bnF, dL, Gw, K, hu, eL, wr, aW, Y, ff, eH, CT, r, GA, dg, aF, La, en, bu, cl, t, cM, bz, zE, g, eF, iK, ck, sS, Dy, Zi, eM, bnE, dB, hk, Pl, afd, vy, cj, hL, at, Lb, dy, m, Gu, Gp, Pk, rA, it); #c1(ERCC5) c2(NP_000114) c3(DNA repair protein complementing XP-G cells) c4(1898, 1899, 1900, 1901) c5(1902, 1903, 1904, 1905, 1906, 1907, 1908, 1909, 1910, 1911, 1912, 1913, 1914, 1915, 1916, 1917, 1918, 1919) c6( )c7(gV, T, os, GI, id, if, iW, aDE, eX, jh, aA, n, oZ, Gn, ci, fr, V, iJ, q, cd, ad, eA, dP, ip, av, hW, Qk, yg, aT, rA, eV, dL, K, bnG, ae, eL, hG, aW, Y, ff, eH, CT, r, hQ, aq, aF, La, en, cl, eF, t, zE, bz, QA, iK, es, Dy, eM, iT, dB, hk, hl, cj, at, m, NB, bt, it, B, rk); #c1(ERCC6) c2(NP_000115) c3(DNA excision repair protein ERCC-6) c4(1920, 1921, 1922, 1923, 1924) c5(1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950) c6( )c7(GI, T, dD, eR, yk, iW, aA, n, Gn, ci, eq, Do, V, q, aR, ad, eA, eW, av, MR, aXH, nl, aT, aYf, dL, KP, K, cx, eL, kj, bnH, aW, Az, nh, r, kt, CT, aF, La, QK, lk, bz, iK, dr, sS, W, nb, U, cj, at, m, rA, xb, B); #c1(ERCCGL2) c2(NP_001010895) c3(DNA excision repair protein ERCC-6-like 2 isoform c) c4( )c5(23996, 23997, 23998, 23999, 24000, 24001, 24002, 24003, 24004, 24005, 24006, 24007, 24008, 24009, 24010, 24011, 24012, 24013) c6( )c7(bnl, oX); #c1(ERCCGL2) c2(NP_064592) c3(DNA excision repair protein ERCC-6-like 2 isoform a) c4(203148) c5(203149, 203150, 203151, 203152, 203153, 203154) c6( )c7(bnl, oX); #c1(ERCC6L2) c2(XP_011516944) c3(DNA excision repair protein ERCC-6-like 2 isoform X2) c4(310475) c5( ) c6( )c7(bnl, oX); #c1(ERCCGL2) c2(XP_011516946) c3(DNA excision repair protein ERCC-6-like 2 isoform X4) c4(310476) c5( )c6( ) c7(bnl, oX); #c1(ERCC6L2) c2(XP_011516952) c3(DNA excision repair protein ERCC-6-like 2 isoform X10) c4(310477) c5( )c6( )c7(bnl, oX); #c1(ERCC6-PGBD3) c2(NP_001263988) c3(Cockayne syndrome B-piggyBac fusion protein) c4(97409) c5(97410, 97411, 97412, 97413, 97414, 97415, 97416, 97417, 97418, 97419, 97420, 97421, 97422, 97423, 97424, 97425) c6( )c7(bj, aYf, La); #c1(ERCC8) c2(NP_000073) c3(DNA excision repair protein ERCC-8 isoform 1) c4(1211, 1212, 1213) c5(1214, 1215, 1216, 1217, 1218, 1219, 1220, 1221, 1222, (223, 1224, 1225, 1226) c6( )c7(ck, aR, T, MR, aXH, jZ, bnJ, aT, dr, U, dz, dL, KP, K, dV, La, lM, V, sH); #c1(ERCC8) c2(NP_001007234) c3(DNA excision repair protein ERCC-8 isoform 2) c4( )c5(22588, 22589, 22590, 22591, 22592, 22593) c6( )c7(ck, aR, T, MR, aXH, jZ, bnJ, aT, dr, U, dz, dL, KP, K, dV, La, lM, V, sH); #c1(ERCC8) c2(NP_001277214) c3(DNA excision repair protein ERCC-8 isoform 4) c4(109233) c5(109234, 109235, 109236, 109237, 109238) c6( )c7(ck, aR, T, MR, aXH, jZ, bnJ, aT, dr, U, dz, dL, KP, K, dV, La, lM, V, sH); #c1(EREG) c2(NP_001423) c3(proepiregulin preproprotein) c4(117757) c5(117758, 117759, 117760, (17761, 117762, 117763, 117764, 117765) c6(117766, 117767) c7(T, hV, aA, ci, nE, asS, q, eA, av, iu, qK, gd, aT, ey, ji, fb, d, Ls, eH, ff, r, aF, en, g, eF, t, KM, eM, m, i, y, B); #c1(ERF) c2(NP_001287964) c3(ETS domain-containing transcription factor ERF isoform 2) c4( )c5(113274, 113275, 113276, 113277, 113278) c6( ) c7(bnK, ck, bnl, bnM, eD, Az, r, hR, fe, aQM, f, bz, q, zV); #c1(ERF) c2(NP_006485) c3(ETS domain-containing transcription factor ERF isoform 1) c4(165618, 165619) c5(165620, 165621, 165622, 165623, 165624, 165625, 165626) c6( )c7(bnK, ck, bnl, bnM, eD, Az, r, hR, fe, aQM, f, bz, q, zV); #c1(ERG) c2(NP_001129626) c3(transcriptional regulator ERG isoform 3) c4(52061) c5(52062, 52063, 52064, 52065, 52066, 52067, 52068, 52069, 52070, 52071) c6( )c7(gh, T, ci, amW, x, bv, xE, SV, dV, Ou, K, aW, bnQ, g, QA, iT, bjY, nw, m, k, cZ, n, Sw, afR, ft, pN, Zs, h, hn, dd, bnO, db, dx, YI, AT, A, RT, AQ, bnN, W, Vy, nx, fZ, aA, pd, q, aT, jj, avt, ae, af, jf, iF, r, gm, en, f, nC, ck, bnR, np, OL, bt, Dd, bnS, Fb, hF, iJ, mC, ad, Jm, F, Sr, iY, uQ, lR, Ub, qt, ff, bgh, aF, Tp, bz, ayj, bgg, EW, bnP, y); #c1(ERG) c2(NP_001129627) c3(transcriptional regulator ERG isoform 4) c4( )c5(52072, 52073, 52074, 52075, 52076) c6( )c7(gh, T, ci, amW, x, bv, xE, SV, dV, Ou, K, aW, bnl, g, LA, iT, bjY, nw, m, k, cZ, n, Sw, afR, ft, pN, Zs, h, hn, dd, bnO, db, dx, YI, AT, A, RT, AQ, bnN, W, Vy, nx, fZ, aA, pd, q, aT, jj, avt, ae, af, jf, iF, r, gm, en, f, nC, ck, bnR, np, OL, bt, Dd, bnS, Fb, hF, iJ, mC, ad, Jm, F, Sr, iY, ul, lR, Ub, qt, ff, bgh, af, Tp, bz, ayj, bgg, EW, bnP, y); #c1(ERG) c2(NP_001230358) c3(transcriptional regulator ERG isoform 5) c4( )c5(86251, 86252, 86253, 86254, 86255) c6( )c7(gh, T, ci, amW, x, bv, xE, SV, dV, Ou, K, aW, bnd, g, DA, iT, bjY, nw, m, k, cZ, n, Sw, afR, ft, pN, Zs, h, hn, dd, bnO, db, dx, YI, AT, A, RT, AB, bnN, W, Vy, nx, fZ, aA, pd, q, aT, jj, avt, ae, af, jf, if, r, gm, en, f, nC, ck, bnR, np, OL, bt, Dd, bnS, Fb, hF, iJ, mC, ad, Jm, F, Sr, iY, ul, lR, Ub, qt, ff, bgh, aF, Tp, bz, ayj, bgg, EW, bnP, y); #c1(ERG) c2(NP_004440) c3(transcriptional regulator ERG isoform 2) c4(146348, 146349) c5(146350, 146351, 146352, 146353, 146354, 146355, 146356, 146357, 146358, 146359, 146360, 146361) c6( )c7(gh, T, ci, amW, x, bv, xE, SV, dV, Ou, K, aW, bnl, g, QA, iT, bjY, nw, m, k, cZ, n, Sw, afR, ft, pN, Zs, h, hn, dd, bnO, db, dx, YI, AT, A, RT, AQ, bnN, W, Vy, nx, fZ, aA, pd, q, aT, jj, avt, ae, af, jf, iF, r, gm, en, f, nC, ck, bnR, np, OL, bt, Dd, bnS, Fb, hF, iJ, mC, ad, Jm, F, Sr, iY, ud, lR, Ub, qt, ff, bgh, aF, Tp, bz, ayj, bgg, EW, bnP, y); #c1(ERG) c2(NP_891548) c3(transcriptional regulator ERG isoform 1) c4(257256, 257257, 257258) c5(257259, 257260, 257261, 257262, 257263, 257264, 257265) c6( )c7(gh, T, ci, amW, x, bv, xE, SV, dV, Ou, K, aW, bnQ, g, QA, iT, bjY, nw, m, k, cZ, n, Sw, afR, ft, pN, Zs, h, hn, dd, bnO, db, dx, YI, AT, A, RT, AQ, bnN, W, Vy, nx, fZ, aA, pd, q, aT, jj, avt, ae, af, jf, iF, r, gm, en, f, nC, ck, bnR, np, OL, bt, Dd, bnS, Fb, hF, iJ, mC, ad, Jm, F, Sr, iY, ul, lR, Ub, qt, ff, bgh, aF, Tp, bz, ayj, bgg, EW, bnP, y); #c1(ERG) c2(XP_011527789) c3(transcriptional regulator ERG isoform X2) c4( )c5(322756, 322757, 322758, 322759, 322760, 322761) c6( )c7(gh, T, ci, amW, x, bv, xE, SV, dV, Ou, K, aW, bnl, g, QA, iT, bjY, nw, m, k, cZ, n, Sw, afR, ft, pN, Zs, h, hn, dd, bnO, db, dx, YI, AT, A, RT, AD, bnN, W, Vy, nx, fZ, aA, pd, q, aT, jj, avt, ae, af, jf, iF, r, gm, en, f, nC, ck, bnR, np, OL, bt, Dd, bnS, Fb, hF, iJ, mC, ad, Jm, F, Sr, iY, uQ, lR, Ub, qt, ff, bgh, af, Tp, bz, ayj, bgg, EW, bnP, y); #c1(ERGIC1) c2(NP_001026881) c3(endoplasmic reticulum-Golgi intermediate compartment protein 1) c4(29821) c5(29822, 29823, 29824, 29825, 29826, 29827) c6( )c7(ae, dy, ac, ad); #c1(ERGIC2) c2(NP_057654) c3(endoplasmic reticulum-Golgi intermediate compartment protein 2) c4(192084, 192085, 192086, 192087, 192088) c5(192089, 192090, 192091, 192092, 192093, 192094, 192095, 192096, 192097, 192098, 192099, 192100, 192101, 192102, 192103, 192104, 192105, 192106, 192107, 192108, 192109, 192110, 192111, 192112, 192113, 192114, 192115, 192116, 192117, 192118, 192119) c6( )c7(s, Xg, hR, ey, ae, ad); #c1(ERGIC3) c2(NP_057050) c3(endoplasmic reticulum-Golgi intermediate compartment protein 3 isoform b) c4(188634, 188635, 188636) c5(188637, 188638, 188639, 188640, 188641, 188642, 188643, 188644, 188645, 188646, 188647, 188648, 188649, 188650, 188651, 188652, 188653, 188654) c6( )c7(iu, bz, B); #c1(ERGIC3) c2(NP_938408) c3(endoplasmic reticulum-Golgi intermediate compartment protein 3 isoform a) c4(259822, 259823) c5(259824, 259825, 259826, 259827, 259828, 259829, 259830, 259831, 259832, 259833, 259834, 259835) c6( )c7(iu, bz, B); #c1(ERI3) c2(NP_001288628) c3(ERI1 exoribonuclease 3 isoform 2) c4(113854, 113855, 113856, 113857, 113858, 113859) c5(113860, 113861, 113862, 113863, 113864, 113865, 113866, 113867, 113868, 113869, 113870, 113871, 113872, 113873, 113874, 113875, 113876, 113877, 113878, 113879, 113880, 113881, 113882, 113883, 113884, 113885, 113886, 113887, 113888, 113889) c6( )c7(ac); #c1(ERI3) c2(NP_001288630) c3(ERI1 exoribonuclease 3 isoform 4) c4( )c5(113890, 113891, 113892, 113893, 113894) c6( )c7(ac); #c1(ERICH5) c2(NP_001164277) c3 (glutamate-rich protein 5 isoform 2) c4(66907) c5(66908, 66909, 66910, 66911, 66912, 66913) c6( )c7(akY); #c1(ERICH5) c2(NP_775820) c3(glutamate-rich protein 5 isoform 1) c4(250404) c5(250405, 250406, 250407, 250408, 250409, 250410) c6( )c7(akY); #c1(ERICH6B) c2(NP_872348) c3(glutamate-rich protein 6B) c4(256169) c5(256170, 256171, 256172, 256173, 256174, 256175) c6( )c7(ac); #c1(ERLEC1) c2(NP_001120869) c3(endoplasmic reticulum lectin I isoform 2 precursor) c4(44879) c5(44880, 44881, 44882, 44883, 44884, 44885) c6( )c7(bz, K); #c1(ERLEC1) c2(NP_001120870) c3(endoplasmic reticulum lectin 1 isoform 3 precursor) c4(44886) c5(44887, 44888, 44889, 44890, 44891, 44892) c6( )c7(bz, K); #c1(ERLEC1) c2(NP_056516) c3(endoplasmic reticulum lectin I isoform 1 precursor) c4(187892) c5(187893, 187894, 187895, 187896, 187897, 187898) c6( )c7(bz, K); #c1(ERLIN1) c2(XP_005269499) c3(erlin-1 isoform X1) c4(282577, 282578, 282579) c5(282580, 282581, 282582, 282583, 282584, 282585, 282586, 282587, 282588, 282589, 282590, 282591, 282592, 282593, 282594, 282595, 282596) c6( )c7(ey, cP, fc); #c1(ERLIN1) c2(XP_011537472) c3(erlin-1 isoform X2) c4( )c5(335082, 335083, 335084, 335085, 335086) c6( )c7(ey, cP, fc); #c1 (ERLIN2) c2(NP_001003791) c3(erlin-2 isoform 2) c4(20100) c5(20101, 20102, 20103, 20104, 20105, 20106, 20107, 20108, 20109, 20110, 20111, 20112, 20113, 20114, 20115, 20116) c6( )c7(T, lu, TL, aW, Pt, axW, bnT, aT, aq, K, nA, B); #c1(ERLIN2) c2(NP_009106) c3(erlin-2 isoform 1) c4(170688, 170689) c5(170690, 170691, 170692, 170693, 170694, 170695, 170696, 170697, 170698, 170699) c6( )c7(T, lu, TL, aW, Pt, axW, bnT, aT, aq, K, nA, B); #c1(ERMAP) c2(XP_011538872) c3(erythroid membrane-associated protein isoform X1) c4(336940, 336941) c5(336942, 336943, 336944, 336945, 336946, 336947, 336948, 336949, 336950, 336951, 336952, 336953, 336954, 336955, 336956, 336957) c6( )c7(bnU, bnV); #c1(ERMARD) c2(NP_001265460) c3(endoplasmic reticulum membrane-associated RNA degradation protein isoform 2) c4(98607) c5(98608, 98609, 98610, 98611, 98612, 98613) c6( )c7(afx, bnW, ha, afz); #c1(ERMARD) c2(NP_001265462) c3(endoplasmic reticulum membrane-associated RNA degradation protein isoform 4) c4(98614) c5(98615, 98616, 98617, 98618, 98619, 98620) c6( )c7(afx, bnW, ha, afz); #c1(ERMARD) c2(NP_060811) c3(endoplasmic reticulum membrane-associated RNA degradation protein isoform 1) c4(197945) c5(197946, 197947, 197948, 197949, 197950, 197951, 197952, 197953) c6( )c7(afx, bnW, ha, afz); #c1(ERMARD) c2(XP_011534242) c3(endoplasmic reticulum membrane-associated RNA degradation protein isoform X2) c4(330584, 330585) c5(330586, 330587, 330588, 330589, 330590, 330591, 330592, 330593, 330594, 330595, 330596) c6( ) c7(afx, bnW, ha, afz); #c1(ERMP1) c2(NP_079172) c3(endoplasmic reticulum metallopeptidase 1) c4(217298, 217299, 217300) c5(217301, 217302, 217303, 217304, 217305, 217306, 217307, 217308, 217309, 217310, 217311) c6( )c7(aXL, an); #c1(ERMP1) c2(XP_011516336) c3(endoplasmic reticulum metallopeptidase 1 isoform X1) c4( )c5(309556, 309557, 309558, 309559, 309560, 309561, 309562, 309563, 309564, 309565, 309566) c6( )c7(aXL, an); #c1(ERN1) c2(NP_001424) c3(serine/threonine-protein kinase/endoribonuclease IRE1 precursor) c4(117768) c5(117769, 117770, 117771, 117772, 117773, 117774) c6( )c7(T, hl, BD, eR, ha, jh, fd, hy, v, eA, hX, aT, jy, K, B, BN, aW, Y, fc, ck, ac, wR, cP, ne); #c1(ERN2) c2(NP_150296) c3(serine/threonine-protein kinase/endoribonuclease IRE2) c4(228070) c5(228071, 228072, 228073, 228074, 228075, 228076) c6( )c7(ao, bM, K, DF); #c1(ERO1L) c2(NP_055399) c3(ERO1-like protein alpha precursor) c4(180790) c5(180791, 180792, 180793, 180794, 180795, 180796) c6( )c7(bL, T, bo, ey); #c1(ERO1LB) c2(NP_063944) c3(ERO1-like protein beta precursor) c4(202359, 202360, 202361, 202362) c5(202363, 202364, 202365, 202366, 202367, 202368, 202369, 202370, 202371, 202372, 202373, 202374, 202375, 202376, 202377, 202378, 202379, 202380, 202381, 202382, 202383, 202384, 202385, 202386) c6( )c7(bM, ac); #c1(ERP29) c2(NP_001029197) c3(endoplasmic reticulum resident protein 29 isoform 2 precursor) c4(30775) c5(30776, 30777, 30778, 30779, 30780) c6( )c7(iu, T, aW, aT, ci, K, B, ad); #c1(ERP29) c2(NP_006808) c3(endoplasmic reticulum resident protein 29 isoform 1 precursor) c4(168284) c5(168285, 168286, 168287, 168288, 168289, 168290, 168291) c6( )c7(iu, T, aW, aT, ci, K, B, ad); #c1(ERP44) c2(NP_055866) c3(endoplasmic reticulum resident protein 44 precursor) c4(184025) c5(184026, 184027, 184028, 184029, 184030, 184031) c6( )c7(ad); #c1(ERV3-1) c2(NP_001007254) c3(endogenous retrovirus group 3 member 1 Env polyprotein precursor) c4(22650) c5(22651, 22652, 22653, 22654, 22655, 22656) c6( )c7(nJ, bnX, eZ, eo, m, bkE, t, V, ae, c1); #c1(ERVW-1) c2(NP_055405) c3(syncytin-1 precursor) c4(180861) c5(180862, 180863, 180864, 180865, 180866, 180867) c6( )c7(T, adP, aiR, eb, gA, boa, ci, aR, dV, ZQ, rB, K, eL, bnY, aW, hl, apn, aB, aoD, g, as, eE, uC, cm, y, aDX, dc, aiM, m, bod, wu, n, kO, eW, Mo, b, pe, kH, cU, Ne, h, afu, aq, CO, kk, jT, dB, NL, eo, fl, agy, B, dA, AL, jx, aDO, ze, V, pd, eA, aaF, hb, aT, bK, rz, ae, xR, vT, oV, Y, Lf, lo, aDm, bu, cl, aoG, t, ck, iM, il, Zg, AH, Uq, boc, rK, gx, Kd, hl, nl, Ke, iW, jh, Hn, pl, jE, EA, mC, ad, XB, aoH, dP, fU, bnZ, hX, F, tT, ds, bob, oW, lQ, wm, nJ, d, bkf, je, wc, gC, eZ, boe, xH, aux, i, HE, EB); #c1(ESAM) c2(NP_620411) c3(endothelial cell-selective adhesion molecule precursor) c4(237586) c5(237587, 237588, 237589, 237590, 237591, 237592) c6( )c7(ea, Y, dD, eq, fD); #c1(ESAM) c2(XP_005271777) c3(endothelial cell-selective adhesion molecule isoform X2) c4(283578) c5(283579, 283580, 283581, 283582, 283583) c6( )c7(ea, Y, dD, eq, fD); #c1(ESCO1) c2(NP_443143) c3(N-acetyltransferase ESCO1) c4(229626) c5(229627, 229628, 229629, 229630, 229631, 229632) c6( )c7(T, ib, bof, aA, cj, cm, aF, bu, f, it, af, ad, c1); #c1(ESCO1) c2(XP_011524101) c3(N-acetyltransferase ESCO1 isoform X2) c4(318661) c5(318662, 318663, 318664, 318665, 318666, 318667) c6( )c7(T, ib, bof, aA, cj, cm, aF, bu, f, it, af, ad, c1); #c1(ESCO2) c2(XP_011542723) c3(N-acetyltransferase ESCO2 isoform X1) c4(341294) c5(341295, 341296, 341297, 341298, 341299, 341300) c6( )c7(T, bei, nX, Az, atS, cj, aNL, Km, nA); #c1(ESC02) c2(XP_011542724) c3(N-acetyltransferase ESCO2 isoform X2) c4(341301) c5(341302, 341303, 341304, 341305, 341306, 341307) c6( )c7(T, bei, nX, Az, atS, cj, aNL, Km, nA); #c1(ESD) c2(XP_011533256) c3(S-formylglutathione hydrolase isoform X1) c4(329435) c5(329436, 329437, 329438, 329439, 329440, 329441) c6( )c7(eY, A, bP, eM, bog, avA, qW, ace, MW, uu, j, bau, dl, kL); #c1(ESF1) c2(NP_001263309) c3(ESF1 homolog) c4(97160, 97161) c5(97162, 97163, 97164, 97165, 97166, 97167, 97168, 97169, 97170, 97171, 97172, 97173, 97174) c6( )c7(cd); #c1(ESM1) c2(NP_001129076) c3(endothelial cell-specific molecule 1 isoform b precursor) c4(50745) c5(50746, 50747, 50748, 50749, 50750, 50751, 50752, 50753, 50754) c6(50755, 50756) c7(es, T, py, iA, al, aA, r, dL, af, q, hE, eL, B); #c1(ESM1) c2(NP_008967) c3(endothelial cell-specific molecule 1 isoform a precursor) c4(169946) c5(169947, 169948, 169949, 169950, 169951, 169952, 169953, 169954) c6(169955, 169956) c7(es, T, py, iA, al, aA, r, dL, aF, q, hE, eL, B); #c1(ESPL1) c2(NP_036423) c3(separin) c4(173715, 173716) c5(173717, 173718, 173719, 173720, 173721, 173722, 173723, 173724, 173725, 173726, 173727, 173728, 173729, 173730, 173731, 173732) c6( )c7(ck, av, T, fM, id, ajm, eM, il, eH, qK, aT, HR, KO, dL, eF, bz, ad); #c1(ESPN) c2(XP_011540537) c3(espin isoform X6) c4(339021) c5(339022, 339023, 339024, 339025, 339026, 339027, 339028, 339029, 339030, 339031, 339032, 339033) c6( )c7(T, Y, aRa, alA, nw, oc, boh, Bh, boi); #c1(ESPN) c2(XP_011540540) c3(espin isoform X9) c4( )c5(339034, 339035, 339036, 339037, 339038, 339039) c6( )c7(T, Y, aRa, alA, nw, oc, boh, Bh, boi); #c1(ESR1) c2(NP_001278159) c3(estrogen receptor isoform 2) c4(109487) c5( )c6( )c7(gh, T, ea, bO, fd, aeu, ci, aY, Hr, bv, bor, aE, ajv, auB, iv, HR, hu, aiJ, azh, li, yo, nY, PQ, zE, GR, nB, cj, cX, k, sr, FG, aj, ml, cd, aow, Dz, dd, mJ, pl, yi, MD, sp, fe, zZ, hq, iO, dB, bom, Be, rp, Dh, bmk, aA, cz, bX, AD, q, aew, akU, rO, eu, bA, NE, iy, rs, eS, cl, pb, pa, ac, aDR, bt, On, iW, jN, jh, rr, uu, ad, hX, avp, bop, yt, le, wm, WZ, lx, cW, PE, ff, aeV, iV, bz, lC, aed, ne, iu, bM, bou, if, e, bB, cl, ur, aTS, ip, fS, anV, aeO, bc, Ur, xQ, JI, hl, Qm, as, eF, cm, vA, mx, iS, alm, Th, in, ik, dD, gU, afA, lT, wu, n, jz, zw, ec, Do, hi, A, ael, PU, jq, Ye, YE, aqp, cA, bP, PI, hL, at, fL, B, qc, vQ, bcq, wl, ti, v, V, asG, HJ, hW, BJ, aql, rE, ae, jf, hM, Qr, vT, ev, Lf, ate, bu, t, KQ, Px, ck, kP, yH, nZ, Dd, eq, hd, hD, vc, nJ, auY, d, NH, rM, Z, HI, bF, gk, yE, ll, gZ, y, pC, bdo, fR, atj, al, Xi, gf, qj, xE, u, Xc, Bc, rJ, pX, ari, M, K, aDZ, eL, aNb, g, uC, ajj, rq, AM, Gs, m, bok, Vo, sw, zX, aw, NT, fN, qy, boj, abQ, zP, Pp, cM, auC, Ld, KT, W, afc, mg, boo, gv, lB, Pr, it, aoU, aAf, boq, oe, bot, jx, j, pd, eA, J, o, er, jF, aHu, qv, qG, bq, atn, aRR, kl, Y, r, en, wA, iK, bN, sn, hc, dy, KY, LS, gx, sY, id, aZ, LX, bS, EA, boy, azk, dP, fU, fX, acu, io, hP, ael, eH, bow, si, iR, auZ, aFb, eB, ei, hE, vt, bol, G, auW, pf, acs, il, iX, qM, dQ, eN, hm, zQ, box, aW, bov, PG, hJ, aGl, eM, iT, sv, pw, cZ, wh, Bm, fr, ct, Ea, qN, ai, eW, hw, zf, h, dL, hB, asT, he, wC, aiB, bos, iw, aq, op, bJ, vb, bm, hR, uz, ZZ, bL, eR, azn, nv, aT, vH, bK, an, af, qw, s, ND, il, ahe, hf, bQ, f, aDQ, pH, sS, nM, pZ, alt, aGo, aTU, im, hS, ha, vr, Fb, jE, aFJ, mC, qC, av, abk, l, ib, Rq, sO, qt, eZ, aF, ek, es, atM, bE, WK, aS, bon, X, atd); #c1(ESR1) c2(NP_001278170) c3 (estrogen receptor isoform 3) c4(109488) c5( )c6( )c7(gh, T, ea, bO, fd, aeu, ci, aY, Hr, bv, bor, aE, ajv, auB, iv, HR, hu, aiJ, azh, li, yo, nY, PQ, ZE, GR, nB, cj, cX, k, sr, FG, aj, ml, cd, aow, Dz, dd, mJ, pl, yi, MD, sp, fe, zZ, hq, iO, dB, bom, Be, rp, Dh, bmk, aA, cz, bX, AD, q, aew, akU, rO, eu, bA, NE, iy, rs, eS, cl, pb, pa, ac, aDR, bt, On, iW, jN, jh, rr, uu, ad, hX, avp, bop, yt, le, wm, WZ, lx, cW, PE, ff, aeV, iV, bz, lC, aed, ne, iu, bM, bou, if, e, bB, cL, ur, aTS, ip, fS, anV, aeO, bc, Ur, xQ, JI, hl, Qm, as, eF, cm, vA, mx, iS, alm, Th, in, ik, dD, gU, afA, lT, wu, n, jz, zw, ec, Do, hi, A, ael, PU, jq, Ye, YE, aqp, cA, bP, Pl, hL, at, fL, B, qc, vQ, bcq, wL, ti, v, V, asG, HJ, hW, BJ, aql, rE, ae, jf, hM, Or, vT, ev, Lf, ate, bu, t, KQ, Px, ck, kP, yH, nZ, Dd, eq, hd, hD, vc, nJ, auY, d, NH, rM, Z, HI, bF, gk, yE, ll, gZ, y, pC, bdo, FR, atj, al, Xi, gf, qj, xE, u, Xc, Bc, rJ, pX, ari, M, K, aDZ, eL, aNb, g, uC, ajj, rq, AM, Gs, m, bok, Vo, sw, zX, aw, NT, fN, qy, boj, abd, zP, Pp, cM, auC, Ld, KT, W, afc, mg, boo, gv, lB, Pr, it, aol, aAf, boq, oe, bot, jx, j, pd, eA, J, o, er, jF, alu, qv, qG, bq, atn, aRR, kl, Y, r, en, wA, iK, bN, sn, hc, dy, KY, LS, gx, sY, id, aZ, LX, bS, EA, boy, azk, dP, fU, fx, acu, io, hP, aed, eH, bow, si, iR, auZ, aFb, eB, ei, hE, vt, bol, G, auW, pf, acs, il, iX, qM, dQ, eN, hm, zQ, box, aW, bov, PG, hJ, aGl, eM, iT, sv, pw, cZ, wh, Bm, fr, ct, Ea, qN, ai, eW, hw, zf, h, dL, hB, asT, he, wC, aiB, bos, iw, aq, op, bJ, vb, bm, hR, uz, ZZ, bL, eR, azn, nv, aT, vH, bK, an, af, qw, s, ND, il, ahe, hf, bQ, f, aDQ, pH, sS, nM, pZ, alt, aGo, aTU, im, hS, ha, vr, Fb, jE, aFJ, mC, qC, av, abk, l, ib, Rq, sO, qt, eZ, aF, ek, es, atM, bE, WK, aS, bon, X, atd); #c1(ESR1) c2(XP_006715437) c3(estrogen receptor isoform X3) c4( )c5(289532, 289533, 289534, 289535, 289536, 289537, 289538, 289539, 289540, 289541, 289542, 289543, 289544, 289545, 289546, 289547, 289548, 289549, 289550, 289551, 289552, 289553, 289554, 289555, 289556, 289557, 289558) c6( )c7(gh, T, ea, bO, fd, aeu, ci, aY, Hr, bv, bor, aE, ajv, auB, iv, HR, hu, aiJ, azh, li, yo, nY, PQ, zE, GR, nB, cj, cX, k, sr, FG, aj, ml, cd, aow, Dz, dd, mJ, pl, yi, MD, sp, fe, zZ, hq, iO, dB, bom, Be, rp, Dh, bmk, aA, cz, bX, AO, q, aew, akU, rO, eu, bA, NE, iy, rs, eS, cl, pb, pa, ac, aDR, bt, On, iW, jN, jh, rr, uu, ad, hX, avp, bop, yt, le, wm, WZ, lx, cW, PE, ff, aeV, iV, bz, lC, aed, ne, iu, bM, bou, if, e, bB, cL, ur, aTS, ip, fS, anV, aeO, bc, Ur, xQ, JI, hl, Qm, as, eF, cm, vA, mx, iS, alm, Th, in, ik, dD, gU, afA, lT, wu, n, jz, zw, ec, Do, hi, A, aeU, PU, jq, Ye, YE, aqp, cA, bP, Pl, hL, at, fL, B, qc, vQ, bcq, wL, ti, v, V, asG, HJ, hW, BJ, aql, rE, ae, jf, hM, Qr, vT, ev, Lf, ate, bu, t, KQ, Px, ck, kP, yH, nZ, Dd, eq, hd, hD, vc, nJ, auY, d, NH, rM, Z, HI, bF, gk, yE, ll, gZ, y, pC, bdo, fR, atj, al, Xi, gf, qj, xE, u, Xc, Bc, rJ, pX, ari, M, K, aDZ, eL, aNb, g, uC, ajj, rq, AM, Gs, m, bok, Vo, sw, zX, aw, NT, fN, qy, boj, abQ, zP, Pp, CM, auC, Ld, KT, W, afc, mg, boo, gv, lB, Pr, it, aoU, aAf, boq, oe, bot, jx, j, pd, eA, J, o, er, jF, aHu, qv, qG, bq, atn, aRR, kl, Y, r, en, wA, iK, bN, sn, hc, dy, KY, LS, gx, sY, id, aZ, LX, bS, EA, boy, azk, dP, fU, fX, acu, io, hP, aed, eH, bow, si, iR, auZ, aFb, eB, ei, hE, vt, bol, G, auW, pf, acs, il, iX, qM, dQ, eN, hm, zQ, box, aW, bov, PG, hJ, aGl, eM, iT, sv, pw, cZ, wh, Bm, fr, ct, Ea, qN, ai, eW, hw, zf, h, dL, hB, asT, he, wC, aiB, bos, iw, aq, op, bJ, vb, bm, hR, uz, ZZ, bL, eR, azn, nv, aT, vH, bK, an, af, qw, s, ND, iU, ahe, hf, bQ, f, aDQ, pH, sS, nM, pZ, alt, aGo, aTU, im, hS, ha, vr, Fb, jE, aFJ, mC, qC, av, abk, l, ib, Rq, sO, qt, eZ, af, ek, es, atM, bE, WK, aS, bon, X, atd); #c1(ESR1) c2(XP_011533848) c3(estrogen receptor isoform X1) c4(330026, 330027, 330028, 330029, 330030, 330031) c5(330032, 330033, 330034, 330035, 330036, 330037, 330038, 330039, 330040, 330041, 330042, 330043, 330044, 330045, 330046, 330047, 330048, 330049, 330050, 330051, 330052, 330053, 330054, 330055, 330056) c6( )c7(gh, T, ea, bO, fd, aeu, ci, aY, Hr, bv, bor, aE, ajv, auB, iv, HR, hu, aiJ, azh, li, yo, nY, PQ, zE, GR, nB, cj, cX, k, sr, FG, aj, ml, cd, aow, Dz, dd, mJ, pl, yi, MD, sp, fe, zZ, hq, iO, dB, bom, Be, rp, Dh, bmk, aA, cz, bX, AO, q, aew, akU, rO, eu, bA, NE, iy, rs, eS, cl, pb, pa, ac, aDR, bt, On, iW, jN, jh, rr, uu, ad, hX, avp, bop, yt, le, wm, WZ, lx, cW, PE, ff, aeV, iV, bz, lC, aed, ne, iu, bM, bou, if, e, bB, cL, ur, aTS, ip, fS, anV, aeO, bc, Ur, xQ, JI, hl, Qm, as, eF, cm, vA, mx, iS, alm, Th, in, ik, dD, gU, afA, lT, wu, n, jz, zw, ec, Do, hi, A, ael, PU, jq, Ye, YE, aqp, cA, bP, Pl, hL, at, fL, B, qc, vQ, bcq, wL, ti, v, V, asG, HJ, hW, BJ, aql, rE, ae, jf, hM, Or, vT, ev, Lf, ate, bu, t, KQ, Px, ck, kP, yH, nZ, Dd, eq, hd, hD, vc, nJ, auY, d, NH, rM, Z, HI, bF, gk, yE, ll, gZ, y, pC, bdo, FR, atj, al, Xi, gf, qj, xE, u, Xc, Bc, rJ, pX, ari, M, K, aDZ, eL, aNb, g, uC, ajj, rq, AM, Gs, m, bok, Vo, sw, zX, aw, NT, fN, qy, boj, abQ, zP, Pp, cM, auC, Ld, KT, W, afc, mg, boo, gv, lB, Pr, it, aol, aAf, boq, oe, bot, jx, j, pd, eA, J, o, er, jF, aHu, qv, qG, bq, atn, aRR, kl, Y, r, en, wA, iK, bN, sn, hc, dy, KY, LS, gx, sY, id, aZ, LX, bS, EA, boy, azk, dP, fU, fX, acu, io, hP, aed, eH, bow, si, iR, auZ, aFb, eB, ei, hE, vt, bol, G, auW, pf, acs, il, iX, qM, dQ, eN, hm, zQ, box, aW, bov, PG, hJ, aGl, eM, iT, sv, pw, cZ, wh, Bm, fr, ct, Ea, qN, ai, eW, hw, zf, h, dL, hB, asT, he, wC, aiB, bos, iw, aq, op, bJ, vb, bm, hR, uz, ZZ, bL, eR, azn, nv, aT, vH, bK, an, af, qw, s, ND, il, ahe, hf, bQ, f, aDQ, pH, sS, nM, pZ, alt, aGo, aTU, im, hS, ha, vr, Fb, jE, aFJ, mC, qC, av, abk, l, ib, Rq, sO, qt, eZ, aF, ek, es, atM, bE, WK, aS, bon, X, atd); #c1(ESR1) c2(XP_011533850) c3(estrogen receptor isoform X4) c4(330057) c5(330058, 330059, 330060, 330061, 330062, 330063, 330064, 330065, 330066, 330067) c6( )c7(gh, T, ea, bO, fd, aeu, ci, aY, Hr, bv, bor, aE, ajv, auB, iv, HR, hu, aiJ, azh, li, yo, nY, PQ, zE, GR, nB, cj, cX, k, sr, FG, aj, ml, cd, aow, Dz, dd, mJ, pl, yi, MD, sp, fe, zZ, hq, iO, dB, bom, Be, rp, Dh, bmk, aA, cz, bX, AO, q, aew, akU, rO, eu, bA, NE, iy, rs, eS, cl, pb, pa, ac, aDR, bt, On, iW, jN, jh, rr, uu, ad, hX, avp, bop, yt, le, wm, WZ, lx, cW, PE, ff, aeV, iV, bz, lC, aed, ne, iu, bM, bou, if, e, bB, cL, ur, aTS, ip, fS, anV, aeO, bc, Ur, xQ, JI, hl, Qm, as, eF, cm, vA, mx, iS, alm, Th, in, ik, dD, gU, afA, lT, wu, n, jz, zw, ec, Do, hi, A, aeU, PU, jq, Ye, YE, aqp, cA, bP, PI, hl, at, fL, B, qc, vl, bcq, wL, ti, v, V, asG, HJ, hW, BJ, aql, rE, ae, jf, hM, Or, vT, ev, Lf, ate, bu, t, KQ, Px, ck, kP, yH, nZ, Dd, eq, hd, hD, vc, nu, auY, d, NH, rM, Z, HI, bF, gk, yE, ll, gZ, y, pC, bdo, FR, atj, al, Xi, gf, qj, xE, u, Xc, Bc, rJ, pX, ari, M, K, aDZ, eL, aNb, g, uC, ajj, rq, AM, Gs, m, bok, Vo, sw, zX, aw, NT, fN, qy, boj, abl, zP, Pp, cM, auC, Ld, kT, W, afc, mg, boo, gv, lB, Pr, it, aoU, aAf, boq, oe, bot, jx, j, pd, eA, J, o, er, jF, aHu, qv, qG, bq, atn, aRR, kl, Y, r, en, wA, iK, bN, sn, hc, dy, KY, LS, gx, sY, id, aZ, LX, bS, EA, boy, azk, dP, fU, fX, acu, io, hP, aed, eH, bow, si, iR, auZ, aFb, eB, ei, hE, vt, bol, G, auW, pf, acs, il, iX, qM, dQ, eN, hm, zQ, box, aW, bov, PG, hJ, aGl, eM, iT, sv, pw, cZ, wh, Bm, fr, ct, Ea, qN, ai, eW, hw, zf, h, dL, hB, asT, he, wC, aiB, bos, iw, aq, op, bJ, vb, bm, hR, uz, ZZ, bL, eR, azn, nv, aT, vH, bK, an, af, qw, s, ND, iU, ahe, hf, bQ, f, aDQ, pH, sS, nM, pZ, alt, aGo, aTU, im, hS, ha, vr, Fb, jE, aFJ, mC, qC, av, abk, l, ib, Rq, sO, qt, eZ, af, ek, es, atM, bE, WK, aS, bon, X, atd); #c1(ESR1) c2(XP_011533851) c3(estrogen receptor isoform X5) c4(330068, 330069) c5(330070, 330071, 330072, 330073, 330074, 330075, 330076, 330077, 330078, 330079, 330080, 330081, 330082, 330083, 330084, 330085, 330086) c6( )c7(gh, T, ea, bO, fd, aeu, ci, aY, Hr, bv, bor, aE, ajv, auB, iv, HR, hu, aiJ, azh, li, yo, nY, PQ, zE, GR, nB, cj, cX, k, sr, FG, aj, ml, cd, aow, Dz, dd, mJ, pl, yi, MD, sp, fe, zZ, hq, iO, dB, bom, Be, rp, Dh, bmk, aA, cz, bX, AD, q, aew, akU, rO, eu, bA, NE, iy, rs, eS, cl, pb, pa, ac, aDR, bt, On, iW, jN, jh, rr, uu, ad, hX, avp, bop, yt, le, wm, WZ, lx, cW, PE, ff, aeV, iV, bz, lC, aed, ne, iu, bM, bou, if, e, bB, cL, ur, aTS, ip, fS, anV, aeO, bc, Ur, xQ, JI, hl, Qm, as, eF, cm, vA, mx, iS, alm, Th, in, ik, dD, gU, afA, lT, wu, n, jz, zw, ec, Do, hi, A, aeU, PU, jq, Ye, YE, aqp, cA, bP, Pl, hL, at, fL, B, qc, vQ, bcq, wL, ti, v, V, asG, HJ, hW, BJ, aql, rE, ae, jf, hM, Or, vT, ev, Lf, ate, bu, t, KQ, Px, ck, kP, yH, nZ, Dd, eq, hd, hD, vc, nJ, auY, d, NH, rM, Z, HI, bF, gk, yE, ll, gZ, y, pC, bdo, FR, atj, al, Xi, gf, qj, xE, u, Xc, Bc, rJ, pX, ari, M, K, aDZ, eL, aNb, g, uC, ajj, rq, AM, Gs, m, bok, Vo, sw, zX, aw, NT, fN, qy, boj, abQ, zP, Pp, cM, auC, Ld, KT, W, afc, mg, boo, gv, lB, Pr, it, aoU, aAf, boq, oe, bot, jx, j, pd, eA, J, o, er, jF, aHu, qv, qG, bq, atn, aRR, kl, Y, r, en, wA, iK, bN, sn, hc, dy, KY, LS, gx, sY, id, aZ, LX, bS, EA, boy, azk, dP, fU, fX, acu, io, hP, ael, eH, bow, si, iR, auZ, aFb, eB, ei, hE, vt, bol, G, auW, pf, acs, il, iX, qM, dQ, eN, hm, zQ, box, aW, bov, PG, hJ, aGl, eM, iT, sv, pw, cZ, wh, Bm, fr, ct, Ea, qN, ai, eW, hw, zf, h, dL, hB, asT, he, wC, aiB, bos, iw, aq, op, bJ, vb, bm, hR, uz, ZZ, bL, eR, azn, nv, aT, vH, bK, an, af, qw, s, ND, iU, ahe, hf, bQ, f, aDQ, pH, sS, nM, pZ, alt, aGo, aTU, im, hS, ha, vr, Fb, jE, aFJ, mC, qC, av, abk, l, ib, Rq, sO, qt, eZ, aF, ek, es, atM, bE, WK, aS, bon, X, atd); #c1(ESR2) c2(NP_001201831) c3(estrogen receptor beta isoform 3) c4(83528) c5(83529, 83530, 83531, 83532, 83533, 83534, 83535, 83536, 83537, 83538, 83539, 83540, 83541, 83542, 83543, 83544) c6( )c7(bM, T, auW, ea, e, al, bB, ur, ci, lN, bv, u, il, iX, M, boB, iv, bbu, K, dQ, pk, eL, eN, aiJ, yo, zQ, dH, JI, aW, Rr, hl, nY, vs, PQ, NR, g, eF, uC, GR, qJ, rq, nB, AM, eM, vA, ZT, U, m, yA, iu, dD, gU, FG, wh, wu, aw, NT, n, fr, ct, ml, abd, zP, jw, cd, ai, boz, b, h, dL, Dz, hB, wC, yi, PX, fe, aq, Pp, PU, cM, Ld, op, hq, bP, Pl, bom, hl, bm, hR, at, gv, Pr, B, it, qc, boA, fM, De, Lx, Nt, aA, cz, v, j, C, AO, q, eA, aHu, HJ, nv, BJ, aT, vH, an, rE, ae, af, eu, bA, jf, qw, hM, kl, ND, vT, nO, oV, Y, Lf, fO, r, cu, rs, en, cl, wA, t, f, ik, pb, bQ, bN, nM, sn, pZ, kP, ac, ll, mf, aTU, EY, im, hS, HQ, nZ, sY, id, aZ, iW, jh, bS, uu, dG, eq, Fb, ad, dP, av, l, ib, s, NS, le, io, hD, vc, hP, wm, ji, afe, qt, PE, ff, eZ, dq, aeV, aF, Z, bz, yE, bE, aeg, aS, ei, i, lC, X, y); #c1(ESR2) c2(NP_001258806) c3(estrogen receptor beta isoform 6) c4(96556) c5(96557, 96558, 96559, 96560, 96561, 96562) c6( )c7(bM, T, auW, ea, e, al, bB, ur, ci, lN, bv, u, il, iX, M, boB, iv, bbu, K, dQ, pk, eL, eN, aiJ, yo, zQ, dH, JI, aW, Rr, hl, nY, vs, PQ, NR, g, eF, uC, GR, qJ, rq, nB, AM, eM, vA, ZT, U, m, yA, iu, dD, gU, FG, wh, wu, aw, NT, n, fr, ct, ml, abd, zP, jw, cd, ai, boz, b, h, dL, Dz, hB, wC, yi, PX, fe, aq, Pp, PU, cM, Ld, op, hq, bP, Pl, bom, hl, bm, hR, at, gv, Pr, B, it, qc, boA, fM, De, Lx, Nt, aA, cz, v, j, C, AO, q, eA, aHu, HJ, nv, BJ, aT, vH, an, rE, ae, af, eu, bA, jf, qw, hM, kl, ND, vT, nO, oV, Y, Lf, fO, r, cu, rs, en, cl, wA, t, f, ik, pb, bQ, bN, nM, sn, pZ, kP, ac, ll, mf, aTU, EY, im, hS, HQ, nZ, sY, id, aZ, iW, jh, bS, uu, dG, eq, Fb, ad, dP, av, l, ib, sO, NS, le, io, hD, vc, hP, wm, ji, afe, qt, PE, ff, eZ, dq, aeV, aF, Z, bz, yE, bE, aeg, aS, ei, i, lC, X, y); #c1(ESR2) c2(NP_001278652) c3(estrogen receptor beta isoform 2) c4(109933, 109934) c5(109935, 109936, 109937, 109938, 109939, 109940, 109941, 109942, 109943, 109944, 109945, 109946, 109947, 109948) c6( )c7(bM, T, auW, ea, e, al, bB, ur, ci, lN, bv, u, il, iX, M, boB, iv, bbu, K, dQ, pk, eL, eN, aid, yo, zQ, dH, JI, aW, Rr, hl, nY, vs, PQ, NR, g, eF, uC, GR, qJ, rq, nB, AM, eM, vA, ZT, U, m, yA, iu, dD, gU, FG, wh, wu, aw, NT, n, fr, ct, ml, abd, zP, jw, cd, ai, boz, b, h, dL, Dz, hB, wC, yi, PX, fe, aq, Pp, PU, cM, Ld, op, hq, bP, Pl, bom, hL, bm, hR, at, gv, Pr, B, it, qc, boA, fM, De, Lx, Nt, aA, cz, v, j, C, AO, q, eA, aHu, HJ, nv, BJ, aT, vH, an, rE, ae, af, eu, bA, jf, qw, hM, kl, ND, vT, nO, oV, Y, Lf, fO, r, cu, rs, en, cl, wA, t, f, ik, pb, bQ, bN, nM, sn, pZ, kP, ac, ll, mf, aTU, EY, im, hS, HQ, nZ, sY, id, aZ, iW, jh, bS, uu, dG, eq, Fb, ad, dP, av, l, ib, sO, NS, le, io, hD, vc, hP, wm, ji, afe, qt, PE, ff, eZ, dq, aeV, aF, Z, bz, yE, bE, aeg, aS, ei, i, lC, X, y); #c1(ESR2) c2(XP_011534847) c3(estrogen receptor beta isoform X2) c4( )c5(331369, 331370, 331371, 331372, 331373, 331374) c6( )c7(bM, T, auW, ea, e, al, bB, ur, ci, lN, bv, u, il, iX, M, boB, iv, bbu, K, dQ, pk, eL, eN, aiJ, yo, zQ, dH, JI, aW, Rr, hl, nY, vs, PQ, NR, g, eF, uC, GR, qJ, rq, nB, AM, eM, vA, ZT, U, m, yA, iu, dD, gU, FG, wh, wu, aw, NT, n, fr, ct, ml, abd, zP, jw, cd, ai, boz, b, h, dL, Dz, hB, wC, yi, PX, fe, aq, Pp, PU, cM, Ld, op, hq, bP, Pl, bom, hl, bm, hR, at, gv, Pr, B, it, qc, boA, fM, De, Lx, Nt, aA, cz, v, j, C, AD, q, eA, aHu, HJ, nv, BJ, aT, vH, an, rE, ae, af, eu, bA, jf, qw, hM, kl, ND, vT, nO, oV, Y, Lf, fO, r, cu, rs, en, cl, wA, t, f, iK, pb, bQ, bN, nM, sn, pZ, kP, ac, Il, mf, aTU, EY, im, hS, HQ, nZ, sY, id, aZ, iW, jh, bS, uu, dG, eq, Fb, ad, dP, av, l, ib, sO, NS, le, io, hD, vc, hP, wm, ji, afe, qt, PE, ff, eZ, dq, aeV, af, Z, bz, yE, bE, aeg, aS, ei, i, lC, X, y); #c1(ESR2) c2(XP_011534848) c3(estrogen receptor beta isoform X1) c4(331375, 331376) c5(331377, 331378, 331379, 331380, 331381, 331382, 331383) c6( )c7(bM, T, auW, ea, e, al, bB, ur, ci, lN, bv, u, il, iX, M, boB, iv, bbu, K, dQ, pk, eL, eN, aiJ, yo, zQ, dH, JI, aW, Rr, hl, nY, vs, PQ, NR, g, eF, uC, GR, qJ, rq, nB, AM, eM, vA, ZT, U, m, yA, iu, dD, gU, FG, wh, wu, aw, NT, n, fr, ct, ml, abd, zP, jw, cd, ai, boz, b, h, dL, Dz, hB, wC, yi, PX, fe, aq, Pp, PU, cM, Ld, op, hq, bP, Pl, bom, hL, bm, hR, at, gv, Pr, B, it, qc, boA, fM, oe, Lx, Nt, aA, cz, v, j, C, AO, q, eA, aHu, HJ, nv, BJ, aT, vH, an, rE, ae, af, eu, bA, jf, qw, hM, kl, ND, vT, nO, oV, Y, Lf, fO, r, cu, rs, en, cl, wA, t, f, iK, pb, bQ, bN, nM, sn, pZ, kP, ac, ll, mf, aTU, EY, im, hS, HQ, nZ, sY, id, aZ, iW, jh, bS, uu, dG, eq, Fb, ad, dP, av, l, ib, sO, NS, le, io, hD, vc, hP, wm, ji, afe, qt, PE, ff, eZ, dq, aeV, aF, Z, bz, yE, bE, aeg, aS, ei, i, lC, X, y); #c1(ESRP1) c2(NP_001030087) c3(epithelial splicing regulatory protein 1 isoform 2) c4(30918) c5(30919, 30920, 30921, 30922, 30923, 30924) c6( )c7(T, aC, yo, aE, s, OG, bE, r, bB, rO, kp, fr, q, JW, X, ad); #c1(ESRP1) c2(NP_001116297) c3(epithelial splicing regulatory protein 1 isoform 4) c4(43732) c5(43733, 43734, 43735, 43736, 43737, 43738) c6( )c7(T, aC, yo, aE, s, OG, bE, r, bB, rO, kp, fr, q, JW, X, ad); #c1(ESRP1) c2(NP_001116298) c3(epithelial splicing regulatory protein 1 isoform 3) c4(43739) c5(43740, 43741, 43742, 43743, 43744, 43745) c6( )c7(T, aC, yo, aE, s, OG, bE, r, bB, rO, kp, fr, q, JW, X, ad); #c1(ESRP1) c2(NP_060167) c3(epithelial splicing regulatory protein 1 isoform 1) c4(194599) c5(194600, 194601, 194602, 194603, 194604, 194605) c6( )c7(T, aC, yo, aE, s, OG, bE, r, bB, rO, kp, fr, q, JW, X, ad); #c1(ESRP2) c2(NP_079215) c3(epithelial splicing regulatory protein 2) c4(217466) c5(217467, 217468, 217469, 217470, 217471, 217472) c6( )c7(T, q, r); #c1(ESRRA) c2(NP_001269379) c3(steroid hormone receptor ERR1 isoform 1) c4(101375, 101376) c5(101377, 101378, 101379, 101380, 101381, 101382, 101383, 101384, 101385, 101386, 101387, 101388, 101389) c6( )c7(T, bB, v, ci, cd, q, ad, gJ, aql, aT, bq, dQ, bA, A, aW, iw, r, dq, aq, g, cl, iR, t, nB, bE, hL, m, ao); #c1(ESRRA) c2(NP_001269380) c3(steroid hormone receptor ERR1 isoform 2) c4(101390) c5(101391, 101392, 101393, 101394, 101395, 101396, 101397, 101398, 101399, 101400, 101401, 101402) c6( )c7(T, bB, v, ci, cd, q, ad, gJ, aql, aT, bq, dQ, bA, A, aW, iw, r, dq, aq, g, cl, iR, t, nB, bE, hL, m, ao); #c1(ESRRB) c2(NP_004443) c3(steroid hormone receptor ERR2) c4(146362, 146363) c5( )c6( )c7(bL, Mb, T, dD, aNA, ea, Kh, eq, ai, q, ad, iu, aE, pN, aT, K, ae, dx, lR, aW, r, cl, t, dB, boC, hL, m, UF, Bh, B); #c1(ESRRB) c2(XP_005267461) c3(steroid hormone receptor ERR2 isoform X5) c4( )c5(280977, 280978, 280979, 280980, 280981, 280982) c6( )c7(bL, Mb, T, dD, aNA, ea, Kh, eq, ai, q, ad, iu, aE, pN, aT, K, ae, dx, lR, aW, r, cl, t, dB, boC, hL, m, UF, Bh, B); #c1(ESRRB) c2(XP_011534855) c3(steroid hormone receptor ERR2 isoform X3) c4(331384) c5(331385, 331386, 331387, 331388, 331389, 331390, 331391, 331392, 331393, 331394) c6( ) c7(bL, Mb, T, dD, aNA, ea, Kh, eq, ai, q, ad, iu, aE, pN, aT, K, ae, dx, lR, aW, r, cl, t, dB, boC, hL, m, UF, Bh, B); #c1(ESRRB) c2(XP_011534856) c3(steroid hormone receptor ERR2 isoform X4) c4(331395) c5(331396, 331397, 331398, 331399, 331400, 331401, 331402, 331403, 331404, 331405, 331406, 331407, 331408, 331409, 331410, 331411, 331412) c6( )c7(bL, Mb, T, dD, aNA, ea, Kh, eq, ai, q, ad, iu, aE, pN, aT, K, ae, dx, lR, aW, r, cl, t, dB, boC, hL, m, UF, Bh, B); #c1(ESRRG) c2(NP_001230436) c3(estrogen-related receptor gamma isoform 5) c4(86287) c5(86288, 86289, 86290, 86291, 86292, 86293, 86294, 86295, 86296, 86297, 86298, 86299, 86300, 86301, 86302) c6( ) c7(bL, aW, ajP, aT, r, cz, dG, K, g, od, Kw, c1); #c1(ESRRG) c2(NP_001230447) c3(estrogen-related receptor gamma isoform 6) c4(86303) c5(86304, 86305, 86306, 86307, 86308, 86309, 86310) c6( )c7(bL, aW, ajP, aT, r, cz, dG, K, g, ol, Kw, c1); #c1(ESRRG) c2(NP_001429) c3(estrogen-related receptor gamma isoform 1) c4(117776, 117777) c5(117778, 117779, 117780, 117781, 117782, 117783, 117784, 117785, 117786, 117787, 117788, 117789, 117790) c6( )c7(bL, aW, ajP, aT, r, cz, dG, K, g, ol, Kw, c1); #c1(ESRRG) c2(NP_996317) c3(estrogen-related receptor gamma isoform 2) c4(263999, 264000, 264001) c5(264002, 264003, 264004, 264005, 264006, 264007, 264008, 264009, 264010, 264011, 264012, 264013, 264014, 264015, 264016, 264017, 264018, 264019, 264020, 264021, 264022, 264023, 264024, 264025, 264026, 264027, 264028, 264029, 264030, 264031, 264032, 264033, 264034, 264035, 264036, 264037, 264038, 264039, 264040) c6( )c7(bL, aW, ajP, aT, r, cz, dG, K, g, od, Kw, c1); #c1(ESRRG) c2(XP_011507568) c3 (estrogen-related receptor gamma isoform X4) c4(297484) c5(297485, 297486, 297487, 297488, 297489) c6( )c7(bL, aW, ajP, aT, r, cz, dG, K, g, ol, Kw, c1); #c1(ESRRG) c2(XP_011507574) c3(estrogen-related receptor gamma isoform X6) c4( )c5(297490, 297491, 297492) c6( )c7(bL, aW, ajP, aT, r, cz, dG, K, g, od, Kw, c1); #c1(ESRRG) c2(XP_011507584) c3(estrogen-related receptor gamma isoform X7) c4(297493) c5(297494, 297495, 297496, 297497, 297498) c6( )c7(bL, aW, ajP, aT, r, cz, dG, K, g, ol, Kw, c1); #c1(ESX1) c2(NP_703149) c3(homeobox protein ESX1) c4(246011) c5(246012, 246013, 246014, 246015, 246016, 246017, 246018, 246019) c6( )c7(T, ND, MP, vH, UI, ci); #c1(ESYT1) c2(NP_001171725) c3(extended synaptotagmin-1 isoform 1) c4(71452) c5(71453, 71454, 71455, 71456, 71457, 71458, 71459, 71460, 71461, 71462) c6( )c7(T, eM); #c1(ESYT1) c2(NP_056107) c3(extended synaptotagmin-1 isoform 2) c4(185733, 185734) c5(185735, 185736, 185737, 185738, 185739, 185740, 185741, 185742, 185743, 185744, 185745, 185746) c6( )c7(T, eM); #c1(ESYT2) c2(NP_065779) c3(extended synaptotagmin-2) c4(205370, 205371, 205372, 205373, 205374) c5(205375, 205376, 205377, 205378, 205379, 205380, 205381, 205382, 205383, 205384, 205385, 205386, 205387, 205388, 205389, 205390, 205391, 205392, 205393, 205394, 205395, 205396, 205397, 205398, 205399, 205400, 205401, 205402, 205403, 205404) c6( )c7(T); #c1(ESYT3) c2(NP_114119) c3(extended synaptotagmin-3) c4(222227) c5(222228, 222229, 222230, 222231, 222232, 222233) c6( )c7(j); #c1(ETAA1) c2(NP_061875) c3(ewing's tumor-associated antigen 1) c4(200881) c5(200882, 200883, 200884, 200885, 200886, 200887) c6( )c7(yY, aw, cd, j); #c1(ETF1) c2(NP_001269114) c3(eukaryotic peptide chain release factor subunit 1 isoform 3) c4(100467) c5(100468, 100469, 100470, 100471, 100472, 100473) c6( )c7(eR, aW, bnM, aT, cl, aY, bz); #c1(ETF1) c2(NP_001278903) c3(eukaryotic peptide chain release factor subunit 1 isoform 2) c4( )c5(110856, 110857, 110858, 110859, 110860) c6( )c7(eR, aW, bnM, aT, cl, aY, bz); #c1(ETF1) c2(NP_004721) c3(eukaryotic peptide chain release factor subunit 1 isoform 1) c4(149073, 149074) c5(149075, 149076, 149077, 149078, 149079, 149080, 149081) c6( ) c7(eR, aW, bnM, aT, cl, aY, bz); #c1(ETFA) c2(NP_000117) c3(electron transfer flavoprotein subunit alpha, mitochondrial isoform a) c4(1951) c5(1952, 1953, 1954, 1955, 1956, 1957) c6( )c7(bL, T, ol, jh, aj, fr, ci, iJ, J, hd, F, ib, aoc, hb, oj, h, By, af, cp, jf, ZH, CN, So, hp, g, cm, nB, y, B); #c1(ETFA) c2(NP_001121188) c3(electron transfer flavoprotein subunit alpha, mitochondrial isoform b) c4(45726) c5(45727, 45728, 45729, 45730, 45731, 45732) c6( )c7(bL, T, ol, jh, aj, fr, ci, iJ, J, hd, F, ib, aoc, hb, oj, h, By, af, cp, jf, ZH, CN, So, hp, g, cm, nB, y, B); #c1 (ETFB) c2(NP_001014763) c3(electron transfer flavoprotein subunit beta isoform 2) c4(25976) c5(25977, 25978, 25979, 25980, 25981, 25982) c6( )c7(T, hd, nB, ol, oj, By, aj, hp, K, cm, af, J); #c1(ETFB) c2(NP_001976) c3(electron transfer flavoprotein subunit beta isoform 1) c4(122801) c5(122802, 122803, 122804, 122805, 122806, 122807) c6( )c7(T, hd, nB, ol, oj, By, aj, hp, K, cm, af, J); #c1(ETFDH) c2(NP_001268666) c3(electron transfer flavoprotein-ubiquinone oxidoreductase, mitochondrial isoform 2) c4(100106) c5(100107, 100108, 100109, 100110, 100111, 100112) c6( )c7(EG, T, mO, hd, nB, aNJ, bo, ol, oj, dq, By, arf, hp, aj, v, du, cm, af, J, MG); #c1(ETFDH) c2(NP_004444) c3(electron transfer flavoprotein-ubiquinone oxidoreductase, mitochondrial isoform 1 precursor) c4(146364) c5(146365, 146366, 146367, 146368, 146369, 146370) c6( )c7(EG, T, mO, hd, nB, aNJ, bo, ol, oj, dq, By, arf, hp, aj, v, du, cm, af, J, MG); #c1(ETHE1) c2(NP_055112) c3(persulfide dioxygenase ETHE1, mitochondrial) c4(179012) c5(179013, 179014, 179015, 179016, 179017, 179018) c6( )c7(IN, aA, uo, So, dq, aF, acJ, B); #c1(ETNK1) c2(NP_001034570) c3(ethanolamine kinase 1 isoform B) c4(31803) c5(31804, 31805, 31806, 31807, 31808, 31809) c6( )c7(wu, ey, wm); #c1(ETNK1) c2(NP_061108) c3(ethanolamine kinase 1 isoform A) c4(199151) c5(199152, 199153, 199154, 199155, 199156, 199157) c6( )c7(wu, ey, wm); #c1(ETNK1) c2(XP_011519042) c3(ethanolamine kinase 1 isoform X1) c4(313536) c5( )c6( )c7(wu, ey, wm); #c1(ETNK1) c2(XP_011519043) c3(ethanolamine kinase 1 isoform X2) c4( )c5(313537, 313538, 313539, 313540, 313541, 313542) c6( )c7(wu, ey, wm); #c1(ETNK2) c2(NP_001284689) c3(ethanolamine kinase 2 isoform 2) c4(112309) c5(112310, 112311, 112312, 112313, 112314, 112315) c6( )c7(cz); #c1(ETNK2) c2(NP_001284690) c3(ethanolamine kinase 2 isoform 3) c4( )c5(112316, 112317, 112318, 112319, 112320) c6( )c7(cz); #c1(ETNK2) c2(NP_001284691) c3(ethanolamine kinase 2 isoform 4) c4(112321) c5(112322, 112323, 112324, 112325, 112326, 112327) c6( )c7(cz); #c1(ETNK2) c2(NP_060678) c3(ethanolamine kinase 2 isoform 1) c4(197113, 197114, 197115) c5(197116, 197117, 197118, 197119, 197120, 197121, 197122, 197123, 197124, 197125, 197126, 197127, 197128) c6( ) c7(cz); #c1(ETNPPL) c2(NP_00140062) c3(ethanolamine-phosphate phospho-lyase isoform 2) c4(60011) c5(60012, 60013, 60014, 60015, 60016, 60017) c6( )c7(uJ, bK, eZ); #c1(ETNPPL) c2(NP_001140099) c3(ethanolamine-phosphate phospho-lyase isoform 3) c4(60018) c5(60019, 60020, 60021, 60022, 60023, 60024) c6( )c7(uJ, bK, eZ); #c1(ETNPPL) c2(NP_112569) c3(ethanolamine-phosphate phospho-lyase isoform 1) c4(221411) c5(221412, 221413, 221414, 221415, 221416, 221417) c6( )c7(uJ, bK, eZ); #c1(ETS1) c2(NP_00137292) c3(protein C-ets-1 isoform 1) c4(55304) c5(55305, 55306, 55307, 55308, 55309, 55310) c6( )c7(bM, T, aC, if, bB, ci, ip, Vp, gJ, aE, ja, K, hH, JI, aW, Ls, HA, g, as, cm, qJ, nB, xD, AM, cj, m, qy, aj, qN, qW, cq, acA, kl, lG, hn, dx, A, op, GU, dB, hL, fL, B, aA, j, pd, q, J, eA, eJ, aT, cy, Fx, bq, ae, Lu, s, NE, il, iZ, Y, r, en, hf, cl, t, Px, ik, ck, iM, kP, dh, nG, np, bt, jh, hF, tK, mC, ad, dP, hd, yt, Yo, hD, fb, Yi, d, je, BO, ff, eZ, aF, hp, bz, bE, bC, y); #c1(ETS1) c2(NP_001155894) c3(protein C-ets-1 isoform 3) c4(61625) c5(61626, 61627, 61628, 61629, 61630, 61631) c6( )c7(bM, T, aC, if, bB, ci, ip, Vp, gJ, aE, ja, K, hH, JI, aW, Ls, HA, g, as, cm, qJ, nB, xD, AM, cj, m, qy, aj, qN, qW, cq, acA, kl, lG, hn, dx, A, op, GU, dB, hL, fL, B, aA, j, pd, q, J, eA, eJ, aT, cy, Fx, bq, ae, Lu, s, NE, il, iZ, Y, r, en, hf, cl, t, Px, ik, ck, iM, kP, dh, nG, np, bt, jh, hF, tK, mC, ad, dP, hd, yt, Yo, hD, fb, Yi, d, je, BO, ff, eZ, aF, hp, bz, bE, bC, y); #c1(ETS1) c2(NP_005229) c3(protein C-ets-1 isoform 2) c4(154251, 154252) c5(154253, 154254, 154255, 154256, 154257, 154258, 154259, 154260, 154261, 154262, 154263, 154264, 154265) c6( )c7(bM, T, aC, if, bB, ci, ip, Vp, gJ, aE, ja, K, hH, JI, aW, Ls, HA, g, as, cm, qJ, nB, xD, AM, cj, m, qy, aj, qN, qW, cq, acA, kl, lG, hn, dx, A, op, GU, dB, hL, fL, B, aA, j, pd, q, J, eA, eJ, aT, cy, Fx, bq, ae, Lu, s, NE, il, iZ, Y, r, en, hf, cl, t, Px, iK, ck, iM, kP, dh, nG, np, bt, jh, hF, tK, mC, ad, dP, hd, yt, Yo, hD, fb, Yi, d, je, BO, ff, eZ, aF, hp, bz, bE, bC, y); #c1(ETS1) c2(XP_011540954) c3(protein C-ets-1 isoform X4) c4(339487) c5(339488, 339489, 339490, 339491, 339492, 339493, 339494, 339495, 339496, 339497) c6( )c7(bM, T, aC, if, bB, ci, ip, Vp, gJ, aE, ja, K, hH, JI, aW, Ls, HA, g, as, cm, qJ, nB, xD, AM, cj, m, qy, aj, qN, qW, cq, acA, kl, lG, hn, dx, A, op, GU, dB, hL, fL, B, aA, j, pd, q, J, eA, eJ, aT, cy, Fx, bq, ae, Lu, s, NE, iU, iZ, Y, r, en, hf, cl, t, Px, iK, ck, iM, kP, dh, nG, np, bt, jh, hF, tK, mC, ad, dP, hd, yt, Yo, HD, fb, Yi, d, je, BO, ff, eZ, aF, hp, bz, bE, bC, y); #c1(ETS2) c2(NP_001243224) c3(protein C-ets-2 isoform 2) c4(90194, 90195, 90196) c5(90197, 90198, 90199) c6( )c7(bM, T, fG, aA, bB, n, bS, j, ct, ci, hF, q, ad, hX, anj, ib, aE, HC, aT, cU, iY, dl, ji, K, nJ, ae, af, eL, d, je, aW, fJ, ff, Az, eZ, r, aF, en, g, Z, t, f, bz, LB, ck, EW, lt, iM, GU, c, bE, eM, m, fD, y, bt, ik, B); #c1(ETS2) c2(XP_005260992) c3 (protein C-ets-2 isoform X1) c4( ) c5(277050, 277051, 277052, 277053, 277054, 277055, 277056, 277057, 277058, 277059, 277060, 277061, 277062, 277063, 277064, 277065, 277066, 277067, 277068, 277069) c6( )c7(bM, T, fG, aA, bB, n, bS, j, ct, ci, hF, q, ad, hX, anj, ib, aE, HC, aT, cU, iY, dL, ji, K, nJ, ae, af, eL, d, je, aW, fJ, ff, Az, eZ, r, aF, en, g, Z, t, f, bz, LB, ck, EW, lt, iM, GU, c, bE, eM, m, fD, y, bt, ik, B); #c1(ETV1) c2(NP_001156619) c3(ETS translocation variant 1 isoform b) c4(61794, 61795) c5(61796, 61797, 61798, 61799, 61800, 61801, 61802, 61803, 61804, 61805, 61806, 61807, 61808, 61809, 61810, 61811, 61812, 61813, 61814) c6( )c7(gh, T, fb, pf, JI, aW, Y, aT, m, g, t, ae, ad); #c1(ETV1) c2(NP_001156622) c3(ETS translocation variant 1 isoform d) c4(61815) c5(61816, 61817, 61818, 61819, 61820, 61821) c6( )c7(gh, T, fb, pf, JI, aW, Y, aT, m, g, t, ae, ad); #c1(ETV1) c2(NP_001156623) c3(ETS translocation variant 1 isoform e) c4(61822) c5(61823, 61824, 61825, 61826, 61827, 61828) c6( )c7(gh, T, fb, pf, JI, aW, Y, aT, m, g, t, ae, ad); #c1(ETV1) c2(NP_001156624) c3(ETS translocation variant 1 isoform f) c4(61829) c5(61830, 61831, 61832, 61833, 61834, 61835) c6( )c7(gh, T, fb, pf, JI, aW, Y, aT, m, g, t, ae, ad); #c1(ETV1) c2(XP_011513470) c3(ETS translocation variant 1 isoform X1) c4(305731, 305732) c5(305733, 305734, 305735, 305736, 305737, 305738, 305739, 305740, 305741, 305742, 305743, 305744, 305745, 305746, 305747, 305748) c6( )c7(gh, T, fb, pf, JI, aW, Y, aT, m, g, t, ae, ad); #c1(ETV1) c2(XP_011513474) c3(ETS translocation variant 1 isoform X2) c4(305749, 305750) c5(305751, 305752, 305753, 305754, 305755, 305756, 305757, 305758, 305759, 305760, 305761, 305762, 305763, 305764) c6( )c7(gh, T, fb, pf, Jl, aW, Y, aT, m, g, t, ae, ad); #c1(ETV3) c2(NP_005231) c3(ETS translocation variant 3 isoform 2) c4(154266) c5(154267, 154268, 154269, 154270, 154271, 154272) c6( )c7(aW, om, cz, cj, aT); #c1(ETV3) c2(XP_006711273) c3(ETS translocation variant 3 isoform X1) c4(285830) c5(285831, 285832, 285833, 285834, 285835, 285836, 285837, 285838) c6( )c7(aW, om, cz, cj, aT); #c1(ETV4) c2(NP_001073143) c3(ETS translocation variant 4 isoform 1) c4(35633, 35634, 35635) c5(35636, 35637, 35638, 35639, 35640, 35641, 35642, 35643, 35644, 35645, 35646, 35647, 35648, 35649, 35650, 35651, 35652, 35653) c6( )c7(gx, T, aC, pf, wu, NT, aA, bB, n, ci, q, ad, eA, av, aE, NS, aT, K, wm, ae, dx, De, qw, eG, boE, JI, aW, Y, ff, r, aF, en, g, t, boD, bz, eM, bE, m, X); #c1(ETV4) c2(NP_001248366) c3(ETS translocation variant 4 isoform 2) c4( )c5(93823, 93824, 93825, 93826, 93827, 93828, 93829, 93830) c6( )c7(gx, T, aC, pf, wu, NT, aA, bB, n, ci, q, ad, eA, av, aE, NS, aT, K, wm, ae, dx, Qe, qw, eG, boE, JI, aW, Y, ff, r, aF, en, g, t, boD, bz, eM, bE, m, X); #c1(ETV4) c2(XP_011522815) c3(ETS translocation variant 4 isoform X1) c4( )c5(317311) c6( )c7(gx, T, aC, pf, wu, NT, aA, bB, n, ci, q, ad, eA, av, aE, NS, aT, K, wm, ae, dx, Qe, qw, eG, boE, JI, aW, Y, ff, r, aF, en, g, t, boD, bz, eM, bE, m, X); #c1(ETV5) c2(NP_004445) c3(ETS translocation variant 5) c4(146371, 146372, 146373, 146374, 146375) c5(146376, 146377, 146378, 146379, 146380, 146381, 146382, 146383, 146384, 146385, 146386, 146387, 146388, 146389, 146390, 146391, 146392, 146393, 146394, 146395, 146396, 146397, 146398, 146399, 146400, 146401, 146402, 146403, 146404, 146405, 146406, 146407, 146408, 146409, 146410) c6( )c7(bL, bM, T, kh, fM, asW, zX, eX, aj, v, ci, Ul, anu, J, ad, av, aT, h, eV, By, boF, K, yb, vx, ae, dx, li, aW, Y, Ls, cl, pt, t, fq, bz, Ye, ck, Ew, dB, hL, m, YH, iu); #c1(ETV6) c2(NP_001978) c3(transcription factor ETV6) c4(122808) c5(122809, 122810, 122811, 122812, 122813, 122814, 122815) c6( )c7(aiu, T, fM, dA, gp, aHx, VT, bs, cz, n, ol, oY, vr, ci, GC, pz, hF, AD, avD, br, ad, ip, afb, x, b, fX, hd, pp, Xe, Bj, oW, KH, aT, dz, hn, iY, jj, bby, an, ae, il, Yl, sb, eG, Nr, Or, et, il, aW, gK, boG, po, An, jh, pm, avG, mo, t, cM, hf, f, dr, bw, Nq, ck, ou, ac, iT, kB, nB, aHB, oy, bt, bnR, pg, akC, dW, oX, k, m, bC, i, Kg, lB, boH); #c1(ETV7) c2(NP_001193964) c3(transcription factor ETV7 isoform 2) c4(83364) c5(83365, 83366, 83367, 83368, 83369, 83370) c6( )c7(ck, T, dz, jh, fd); #c1(ETV7) c2(NP_001193965) c3(transcription factor ETV7 isoform 3) c4(83371, 83372) c5(83373, 83374, 83375, 83376, 83377, 83378) c6( )c7(ck, T, dz, jh, fd); #c1(ETV7) c2(NP_001193966) c3(transcription factor ETV7 isoform 4) c4( )c5(83379) c6( )c7(ck, T, dz, jh, fd); #c1(ETV7) c2(NP_001193967) c3(transcription factor ETV7 isoform 5) c4(83380) c5(83381, 83382, 83383, 83384, 83385, 83386) c6( )c7(ck, T, dz, jh, fd); #c1(ETV7) c2(NP_001193970) c3 (transcription factor ETV7 isoform 8) c4( )c5(83387, 83388, 83389, 83390, 83391) c6( )c7(ck, T, dz, jh, fd); #c1(ETV7) c2(NP_057219) c3(transcription factor ETV7 isoform 1) c4(189546) c5(189547, 189548, 189549, 189550, 189551, 189552, 189553) c6( )c7(ck, T, dz, jh, fd); #c1(EVA1A) c2(NP_115557) c3(protein eva-1 homolog A) c4(223161, 223162, 223163) c5(223164, 223165, 223166, 223167, 223168, 223169, 223170, 223171, 223172, 223173, 223174, 223175, 223176, 223177, 223178, 223179, 223180, 223181, 223182, 223183, 223184, 223185, 223186, 223187, 223188, 223189, 223190, 223191, 223192, 223193) c6( )c7(T, fq, aw, yA); #c1(EVA1C) c2(NP_478067) c3(protein eva-1 homolog C isoform 1 precursor) c4(230887) c5(230888, 230889, 230890, 230891, 230892, 230893) c6( )c7(aot, aw, g, Ro); #c1(EVC) c2(NP_714928) c3(ellis-van Creveld syndrome protein) c4(246651) c5(246652, 246653, 246654, 246655, 246656, 246657, 246658, 246659, 246660, 246661) c6( )c7(Na, tJ, apM, aDH, rD, bS, bol, Nc, rz, boJ, ad); #c1(EVC2) c2(NP_001159608) c3(limbin isoform 2) c4(64470) c5(64471, 64472, 64473, 64474, 64475) c6( )c7(Na, A1, ln, Nc, bol, boJ); #c1(EVC2) c2(NP_667338) c3(limbin isoform 1) c4(242006, 242007) c5(242008, 242009, 242010, 242011, 242012, 242013, 242014, 242015, 242016, 242017, 242018, 242019, 242020) c6( )c7(Na, A1, ln, Nc, bol, boJ); #c1(EVI2A) c2(NP_001003927) c3(protein EVI2A isoform 1) c4(20282) c5(20283, 20284, 20285, 20286, 20287, 20288, 20289, 20290, 20291) c6( ) c7(ey, V, hP, Ew); #c1(EVI2A) c2(NP_055025) c3(protein EVI2A isoform 2 precursor) c4(178352, 178353) c5(178354, 178355, 178356, 178357, 178358, 178359, 178360, 178361, 178362, 178363, 178364, 178365, 178366) c6( )c7(ey, V, hP, Ew); #c1(EVI2B) c2(NP_006486) c3(protein EVI2B precursor) c4( )c5(165627, 165628, 165629, 165630, 165631) c6( )c7(ey); #c1(EVI2B) c2(XP_005258003) c3(protein EVI2B isoform X2) c4(275449) c5(275450, 275451, 275452, 275453, 275454, 275455) c6( )c7(ey); #c1(EVI2B) c2(XP_011522817) c3(protein EVI2B isoform X1) c4(317312) c5(317313) c6( )c7(ey); #c1(EVI5) c2(XP_011540402) c3(ecotropic viral integration site 5 protein homolog isoform X3) c4(338912) c5( )c6( )c7(ck, cm, V, Y); #c1(EVI5) c2(XP_011540411) c3(ecotropic viral integration site 5 protein homolog isoform X11) c4( )c5(338913, 338914, 338915, 338916, 338917, 338918) c6( )c7(ck, cm, V, Y); #c1(EVL) c2(NP_057421) c3(ena/VASP-like protein) c4(190697, 190698, 190699) c5(190700, 190701, 190702, 190703, 190704, 190705, 190706, 190707, 190708, 190709, 190710, 190711) c6( )c7(q, r, aT); #c1(EVL) c2(XP_005267806) c3(ena/VASP-like protein isoform X1) c4(281252) c5(281253, 281254, 281255, 281256, 281257, 281258) c6( )c7(q, r, aT); #c1(EVL) c2(XP_011535129) c3(ena/VASP-like protein isoform X3) c4(331964) c5(331965, 331966, 331967, 331968, 331969, 331970, 331971) c6( )c7(q, r, aT); #c1(EVL) c2(XP_011535131) c3(ena/VASP-like protein isoform X5) c4( )c5(331972, 331973, 331974, 331975, 331976, 331977) c6( )c7(q, r, aT); #c1(EVPL) c2(NP_001979) c3(envoplakin) c4(122816) c5(122817, 122818, 122819, 122820, 122821, 122822) c6( )c7(dD, dA, ow, ea, gp, ht, DE, OD, by, eq, hF, br, x, fx, oC, oN, dz, oF, hn, ox, iY, hu, il, il, et, axu, baP, bw, ck, ay, ou, pc, bP, iT, k, bt); #c1(EVX1) c2(NP_001291449) c3(homeobox even-skipped homolog protein 1 isoform 2) c4( )c5(116062, 116063, 116064, 116065, 116066) c6( )c7(n, T, ae, ad); #c1(EVX1) c2(NP_001980) c3(homeobox even-skipped homolog protein 1 isoform 1) c4(122823, 122824) c5(122825, 122826, 122827, 122828, 122829, 122830, 122831, 122832) c6( )c7(n, T, ae, ad); #c1(EVX2) c2(XP_011509410) c3(homeobox even-skipped homolog protein 2 isoform X1) c4(299959) c5(299960, 299961, 299962, 299963, 299964, 299965, 299966, 299967) c6( )c7(aHC, en, ff, g, arA); #c1(EWSR1) c2(NP_001156757) c3(RNA-binding protein EWS isoform 3) c4(61879) c5(61880, 61881, 61882, 61883, 61884, 61885, 61886, 61887, 61888, 61889, 61890, 61891) c6( )c7(Fv, T, kh, aiH, aGn, aNA, bok, YU, iB, zt, aHA, boM, OH, ci, lu, hF, iJ, er, qH, bv, av, ib, Rq, oG, aWi, avA, SV, yg, Ou, ahK, on, dx, jf, if, eG, oV, Y, Ls, eH, hQ, bol, eF, cm, adj, QA, aVz, ck, es, bnN, QG, YV, bP, GU, c, au, U, qF, Xu, aiG, bt); #c1(EWSR1) c2(NP_001156758) c3(RNA-binding protein EWS isoform 4) c4(61892) c5(61893, 61894, 61895, 61896, 61897, 61898, 61899, 61900, 61901, 61902, 61903, 61904, 61905, 61906, 61907, 61908, 61909, 61910, 61911, 61912, 61913, 61914, 61915, 61916, 61917) c6( )c7(Fv, T, kh, aiH, aGn, aNA, bok, YU, iB, zt, aHA, boM, OH, ci, lu, hF, iJ, er, qH, bv, av, ib, Rq, OG, aWi, avA, SV, yg, Ou, ahK, on, dx, jf, iF, eG, oV, Y, Ls, BH, hQ, bol, eF, cm, adj, QA, aVz, ck, es, bnN, QG, YV, bP, GU, c, au, U, qF, Xu, aiG, bt); #c1(EWSR1) c2(NP_001156759) c3(RNA-binding protein EWS isoform 5) c4(61918) c5(61919, 61920, 61921, 61922, 61923, 61924, 61925, 61926, 61927, 61928, 61929, 61930) c6( )c7(Fv, T, kh, aiH, aGn, aNA, bok, YU, iB, zt, aHA, boM, OH, ci, lu, hF, iJ, er, qH, bv, av, ib, Rq, OG, aWi, avA, SV, yg, Ou, ahK, on, dx, jf, iF, eG, oV, Y, Ls, eH, hQ, bol, eF, cm, adj, QA, aVz, ck, es, bnN, QG, YV, bP, GU, c, au, U, qF, Xu, aiG, bt); #c1(EWSR1) c2(NP_005234) c3(RNA-binding protein EWS isoform 2) c4(154280, 154281) c5( )c6( )c7(Fv, T, kh, aiH, aGn, aNA, bok, YU, iB, zt, aHA, boM, OH, ci, lu, hF, iJ, er, qH, bv, av, ib, Rq, OG, aWi, avA, SV, yg, Ou, ahK, on, dx, jf, iF, eG, oV, Y, Ls, eH, hQ, bol, eF, cm, adj, QA, aVz, ck, es, bnN, QG, YV, bP, GU, c, au, U, qF, Xu, aiG, bt); #c1(EWSR1) c2(NP_053733) c3(RNA-binding protein EWS isoform 1) c4(177265) c5( )c6( )c7(Fv, T, kh, aiH, aGn, aNA, bok, YU, iB, zt, aHA, boM, OH, ci, lu, hF, iJ, er, qH, bv, av, ib, Rq, OG, aWi, avA, SV, yg, Ou, ahK, on, dx, jf, iF, eG, oV, Y, Ls, eH, hQ, bol, eF, cm, adj, DA, aVz, ck, es, bnN, QG, YV, bP, GU, c, au, U, qF, Xu, aiG, bt); #c1(EWSR1) c2(XP_011528304) c3(RNA-binding protein EWS isoform X10) c4(323373, 323374) c5( )c6( )c7(Fv, T, kh, aiH, aGn, aNA, bok, YU, iB, zt, aHA, boM, OH, ci, lu, hF, iJ, er, qH, bv, av, ib, Rq, OG, aWi, avA, SV, yg, Ou, ahK, on, dx, jf, if, eG, oV, Y, Ls, eH, hQ, bol, eF, cm, adj, QA, aVz, ck, es, bnN, QG, YV, bP, GU, c, au, U, qF, Xu, aiG, bt); #c1(EXD2) c2(NP_001180292) c3(exonuclease 3′-5′ domain-containing protein 2 isoform 1) c4(73744, 73745, 73746, 73747) c5(73748, 73749, 73750, 73751, 73752, 73753, 73754, 73755, 73756, 73757, 73758, 73759, 73760, 73761, 73762, 73763, 73764, 73765, 73766, 73767, 73768) c6( )c7(an); #c1(EXD2) c2(XP_011535210) c3(exonuclease 3′-5′ domain-containing protein 2 isoform X1) c4( )c5(332023, 332024, 332025, 332026, 332027, 332028, 332029, 332030, 332031, 332032, 332033, 332034, 332035, 332036, 332037, 332038, 332039, 332040, 332041, 332042, 332043, 332044) c6( )c7(an); #c1(EXD3) c2(NP_060290) c3(exonuclease mut-7 homolog isoform a) c4(195209, 195210) c5(195211, 195212, 195213, 195214, 195215, 195216, 195217, 195218, 195219, 195220, 195221, 195222) c6( )c7(wr); #c1(EX01) c2(NP_003677) c3(exonuclease 1 isoform a) c4(140118, 140119) c5(140120, 140121, 140122, 140123, 140124, 140125) c6( )c7(T, os, aC, oO, aA, oZ, bX, V, q, av, arV, aT, dL, d, BO, Y, r, aq, g, iR, bz, boN, dB, wB, cj, at, gv, m, B); #c1(EX01) c2(XP_011542624) c3(exonuclease I isoform X1) c4(341188) c5(341189, 341190, 341191, 341192, 341193, 341194, 341195, 341196, 341197, 341198, 341199, 341200, 341201) c6( ) c7(T, os, aC, QO, aA, oZ, bX, V, q, av, arV, aT, dL, d, BO, Y, r, aq, g, iR, bz, boN, dB, wB, cj, at, gv, m, B); #c1(EX01) c2(XP_011542626) c3(exonuclease 1 isoform X3) c4(34/202) c5(341203, 341204, 34/205, 341206, 341207, 34/208) c6( )c7(T, os, aC, oO, aA, oZ, bX, V, q, av, arV, aT, dL, d, BO, Y, r, aq, g, iR, bz, boN, dB, wB, cj, at, gv, m, B); #c1(EX01) c2(XP_011542629) c3(exonuclease 1 isoform X6) c4( ) c5(341209, 341210, 341211, 341212, 341213, 34/214) c6( )c7(T, os, aC, oO, aA, oZ, bX, V, q, av, arV, aT, dL, d, BO, Y, r, aq, g, iR, bz, boN, dB, wB, cj, at, gv, m, B); #c1(EXOC1) c2(NP_001020095) c3(exocyst complex component 1 isoform 1) c4(28135) c5(28136, 28137, 28138, 28139, 28140, 28141, 28142, 28143, 28144, 28145) c6( )c7(boO); #c1(EXDC1) c2(XP_005265807) c3(exocyst complex component 1 isoform X1) c4(279733) c5(279734, 279735, 279736, 279737, 279738, 279739) c6( )c7(boO); #c1(EX0C2) c2(NP_060773) c3(exocyst complex component 2) c4(197698, 197699, 197700) c5(197701, 197702, 197703, 197704, 197705, 197706, 197707, 197708, 197709, 197710, 197711, 197712, 197713, 197714, 197715, 197716, 197717, 197718) c6( )c7(T, sS, aE); #c1(EXOC3LI) c2(NP_84861 l) c3(exocyst complex component 3-like protein) c4(253956, 253957, 253958) c5(253959, 253960, 253961, 253962, 253963, 253964, 253965, 253966, 253967, 253968, 253969, 253970, 253971, 253972, 253973, 253974, 253975, 253976) c6( )c7(j); #c1(EXDC3L2) c2(NP_612635) c3(exocyst complex component 3-like protein 2) c4(236718) c5(236719, 236720, 236721, 236722, 236723, 236724, 236725, 236726, 236727, 236728) c6( )c7(bL, B, af); #c1(EXDC4) c2(NP_001032203) c3(exocyst complex component 4 isoform b) c4(31078) c5(31079, 31080, 31081, 31082, 31083, 31084) c6( )c7(bL, c, oe, ff, en, y); #c1(EXDC4) c2(NP_068579) c3(exocyst complex component 4 isoform a) c4(209654, 209655, 209656, 209657, 209658) c5(209659, 209660, 209661, 209662, 209663, 209664, 209665, 209666, 209667, 209668, 209669, 209670, 209671, 209672, 209673, 209674, 209675, 209676, 209677, 209678, 209679, 209680, 209681, 209682, 209683, 209684, 209685, 209686, 209687, 209688) c6( )c7(bL, c, oe, ff, en, y); #c1(EXOC5) c2(NP_006535) c3(exocyst complex component 5) c4(166068, 166069) c5(166070, 166071, 166072, 166073, 166074, 166075, 166076, 166077, 166078, 166079, 166080, 166081) c6( )c7(y, K); #c1(EXOC7) c2(NP_001013861) c3(exocyst complex component 7 isoform 1) c4(25771, 25772) c5(25773, 25774, 25775, 25776, 25777, 25778, 25779, 25780, 25781, 25782, 25783, 25784, 25785, 25786, 25787, 25788) c6( )c7(T, AH, boP); #c1(EXDC7) c2(NP_001138769) c3(exocyst complex component 7 isoform 4) c4(57628) c5(57629, 57630, 57631, 57632, 57633, 57634) c6( )c7(T, AH, boP); #c1(EXDC7) c2(NP_001138770) c3(exocyst complex component 7 isoform 5) c4(57635) c5(57636, 57637, 57638, 57639, 57640, 57641) c6( )c7(T, AH, boP); #c1(EXOC7) c2(NP_001138771) c3(exocyst complex component 7 isoform 6) c4(57642) c5(57643, 57644, 57645, 57646, 57647, 57648) c6( )c7(T, AH, boP); #c1(EXOC7) c2(NP_001269243) c3(exocyst complex component 7 isoform 7) c4(100910) c5(100911, 100912, 100913, 100914, 100915, 100916) c6( )c7(T, AH, boP); #c1(EXOC7) c2(NP_056034) c3(exocyst complex component 7 isoform 2) c4(185221, 185222, 185223) c5(185224, 185225, 185226, 185227, 185228, 185229, 185230, 185231, 185232, 185233, 185234, 185235) c6( )c7(T, AH, boP); #c1(EXOC7) c2(XP_011522874) c3(exocyst complex component 7 isoform X6) c4( ) c5(317349, 317350, 317351, 317352, 317353, 317354) c6( )c7(T, AH, boP); #c1(EXOG) c2(NP_001138936) c3(nuclease EXOG, mitochondrial isoform 2 precursor) c4(58342) c5(58343, 58344, 58345, 58346, 58347, 58348) c6( )c7(bL, Ea); #c1(EXOG) c2(NP_005098) c3(nuclease EXOG, mitochondrial isoform 1 precursor) c4(152852) c5(152853, 152854, 152855, 152856, 152857, 152858, 152859) c6( )c7(bL, Ea); #c1(EXOG) c2(XP_006713510) c3(nuclease EXOG, mitochondrial isoform X2) c4(287177) c5(287178, 287179, 287180, 287181, 287182) c6( )c7(bL, Ea); #c1(EXOSC1) c2(NP_057130) c3(exosome complex component CSL4) c4(189046, 189047, 189048) c5(189049, 189050, 189051, 189052, 189053, 189054, 189055, 189056, 189057, 189058, 189059, 189060, 189061, 189062, 189063, 189064, 189065, 189066) c6( )c7(fe); #c1(EXOSC2) c2(NP_001269637) c3(exosome complex component RRP4 isoform 2) c4(102334, 102335) c5(102336, 102337, 102338, 102339, 102340, 102341, 102342, 102343, 102344, 102345, 102346, 102347) c6( ) c7(aq); #c1(EXOSC2) c2(NP_001269638) c3(exosome complex component RRP4 isoform 3) c4(102348) c5(102349, 102350, 102351, 102352, 102353, 102354) c6( )c7(aq); #c1(EXOSC2) c2(NP_055100) c3(exosome complex component RRP4 isoform 1) c4(178927, 178928, 178929) c5(178930, 178931, 178932, 178933, 178934, 178935, 178936, 178937, 178938, 178939, 178940, 178941, 178942, 178943, 178944, 178945, 178946, 178947) c6( )c7(aq); #c1(EXOSC3) c2(NP_001002269) c3(exosome complex component RRP40 isoform 2) c4(19537) c5(19538, 19539, 19540, 19541, 19542, 19543) c6( )c7(d, bol, Ol, hc, ha, boR, h, UR, wR, aFU, ip); #c1(EXOSC3) c2(NP_057126) c3(exosome complex component RRP40 isoform 1) c4(189021) c5(189022, 189023, 189024, 189025, 189026, 189027) c6( )c7(d, boQ, Ol, hc, ha, boR, h, UR, wR, aFU, ip); #c1(EXOSC4) c2(NP_061910) c3(exosome complex component RRP41) c4(201161) c5(201162, 201163, 201164, 201165, 201166, 201167) c6( )c7(bL, T, aE, e, r, eb, aj, mf, cS, j, v, ei, fr, nS, q, J, c1); #c1(EXOSC5) c2(NP_064543) c3(exosome complex component RRP46) c4(202906) c5(202907, 202908, 202909, 202910, 202911, 202912) c6( )c7(ck, il, aA, ci, bu, bt); #c1(EXOSC6) c2(NP_478126) c3(exosome complex component MTR3) c4(230972) c5(230973, 230974, 230975, 230976, 230977, 230978) c6( )c7(aW, Ei, U, aT, bO, bS, bK, K, ct, Z, he, ra, hq); #c1(EXOSC7) c2(NP_055819) c3(exosome complex component RRP42) c4(183703) c5(183704, 183705, 183706, 183707, 183708, 183709) c6( )c7(ck, T, iM, aE, aDs, je, asP, aBq, cd, agf, K, aan, eg, B, aGW, boS); #c1(EXOSC8) c2(NP_852480) c3(exosome complex component RRP43) c4(255026, 255027) c5(255028, 255029, 255030, 255031, 255032, 255033, 255034, 255035, 255036, 255037, 255038, 255039) c6( )c7(A, oV, h, hN, By, ai); #c1(EXPH5) c2(NP_055880) c3(exophilin-5) c4(184149, 184150) c5(184151, 184152, 184153, 184154, 184155, 184156, 184157) c6( )c7(boT, boU); #c1(EXT1) c2(NP_000118) c3(exostosin-1) c4(1958) c5(1959, 1960, 1961, 1962, 1963, 1964) c6( )c7(T, kh, aw, boZ, NT, fd, asM, n, ah, nA, Qo, bpb, aoH, hX, boY, lE, zw, tT, yg, aT, h, an, Wr, he, bA, Dg, OV, TY, aW, hl, aSt, atf, asV, gG, eF, boW, cm, dr, bN, ck, y, kg, dc, boV, Fk, bim, box, hE, bpa, B, hv, vi); #c1 (EXT2) c2(NP_000392) c3(exostosin-2 isoform 1) c4(8430, 8431) c5(8432, 8433, 8434, 8435, 8436, 8437, 8438, 8439, 8440, 8441, 8442, 8443, 8444, 8445, 8446, 8447, 8448) c6( )c7(bL, T, Dg, kh, lE, TY, pN, kg, zw, boV, yg, h, aj, dG, j, v, Wr, bpa, dr); #c1(EXT2) c2(NP_001171554) c3(exostosin-2 isoform 3) c4(70570) c5(70571, 70572, 70573, 70574, 70575, 70576) c6( )c7(bL, T, Dg, kh, lE, TY, pN, kg, zw, boV, yg, h, aj, dG, j, v, Wr, bpa, dr); #c1(EXT2) c2(NP_997005) c3(exostosin-2 isoform 2) c4( )c5(264939, 264940, 264941, 264942, 264943, 264944, 264945, 264946, 264947, 264948) c6( )c7(bL, T, Dg, kh, lE, TY, pN, kg, zw, boV, yg, h, aj, dG, j, v, Wr, bpa, dr); #c1(EXT2) c2(XP_011518252) c3(exostosin-2 isoform X1) c4( )c5(312278) c6( )c7(bL, T, Dg, kh, lE, TY, pN, kg, zw, boV, yg, h, aj, dG, j, v, Wr, bpa, dr); #c1(EXT2) c2(XP_011518253) c3(exostosin-2 isoform X2) c4(312279, 312280) c5( )c6( )c7(bL, T, Dg, kh, lE, TY, pN, kg, zw, boV, yg, h, aj, dG, j, v, Wr, bpa, dr); #c1(EXTL2) c2(XP_011539298) c3(exostosin-like 2 isoform X3) c4( )c5(337362) c6( )c7(bpc, Ly, kg); #c1(EXTL2) c2(XP_011539300) c3(exostosin-like 2 isoform X4) c4(337363, 337364) c5(337365, 337366, 337367, 337368, 337369) c6( )c7(bpc, Ly, kg); #c1(EXTL3) c2(NP_001431) c3(exostosin-like 3) c4(117791, 117792) c5(117793, 117794, 117795, 117796, 117797, 117798, 117799, 117800, 117801, 117802, 117803, 117804) c6( )c7(DA, Dg, d, kg, aW, bpc, aT, cR, r, j, bz, q); #c1(EYA1) c2(NP_000494) c3 (eyes absent homolog 1 isoform 1) c4(9923) c5(9924, 9925, 9926, 9927, 9928, 9929, 9930, 9931, 9932, 9933, 9934) c6( )c7(bL, bcX, bpe, yQ, wG, aW, arH, De, RX, Az, aMT, aT, aA, aNy, bpf, Pp, bD, bpd, bz, iu, er); #c1(EYA1) c2(NP_742056) c3(eyes absent homolog 1 isoform 3) c4(247911) c5(247912, 247913, 247914, 247915, 247916, 247917) c6( )c7(bL, bcX, bpe, yQ, wG, aW, arH, oe, RX, Az, aMT, aT, aA, aNy, bpf, Pp, bD, bpd, bz, iu, er); #c1(EYA1) c2(NP_742057) c3(eyes absent homolog 1 isoform 2) c4(247918, 247919) c5(247920, 247921, 247922, 247923, 247924, 247925, 247926, 247927, 247928, 247929, 247930, 247931) c6( )c7(bL, bcX, bpe, yQ, wG, aW, arH, oe, RX, Az, aMT, aT, aA, aNy, bpf, Pp, bD, bpd, bz, iu, er); #c1(EYA2) c2(NP_005235) c3(eyes absent homolog 2 isoform a) c4(154282, 154283, 154284) c5(154285, 154286, 154287, 154288, 154289, 154290, 154291, 154292, 154293, 154294, 154295, 154296, 154297, 154298, 154299, 154300, 154301, 154302) c6( )c7(T, yQ, aE, aW, aT, m, g, t, eJ); #c1(EYA2) c2(NP_742108) c3(eyes absent homolog 2 isoform c) c4(248167) c5(248168, 248169, 248/70, 248171, 248172, 248173) c6( )c7(T, yQ, aE, aW, aT, m, g, t, eJ); #c1(EYA4) c2(NP_004091) c3(eyes absent homolog 4 isoform a) c4(143497) c5(143498, 143499, 143500, 143501, 143502, 143503) c6( )c7(T, bph, oe, ht, ci, cd, q, av, cc, yQ, Bh, hP, hu, ZW, ge, r, g, iV, bz, bpg, eM, aBU, mG, mx, it); #c1(EYA4) c2(NP_742101) c3(eyes absent homolog 4 isoform b) c4(248108) c5(248109, 248110, 248111, 248112, 248113, 248114) c6( )c7(T, bph, oe, ht, ci, cd, q, av, cc, yQ, Bh, hP, hu, ZW, ge, r, g, iV, bz, bpg, eM, aBU, mG, mx, it); #c1(EYA4) c2(NP_742103) c3(eyes absent homolog 4 isoform d) c4(248115) c5(248116, 248117, 248118, 248119, 248120, 248121) c6( )c7(T, bph, oe, ht, ci, cd, q, av, cc, yQ, Bh, hP, hu, ZW, ge, r, g, iV, bz, bpg, eM, aBU, mG, mx, it); #c1(EYS) c2(NP_001136272) c3(protein eyes shut homolog isoform 1 precursor) c4(54750) c5(54751, 54752, 54753, 54754, 54755, 54756) c6( )c7(IR, bpi, nw, nk, nC, ayj, ad); #c1(EYS) c2(NP_001136273) c3(protein eyes shut homolog isoform 2 precursor) c4(54757) c5(54758, 54759, 54760, 54761, 54762, 54763) c6( ) c7(IR, bpi, nw, nk, nC, ayj, ad); #c1(EYS) c2(NP_001278938) c3(protein eyes shut homolog isoform 4 precursor) c4(111075, 111076) c5(111077, 111078, 111079, 111080, 111081, 111082, 111083, 111084, 111085, 111086) c6( )c7(IR, bpi, nw, nk, nC, ayj, ad); #c1(EYS) c2(NP_938024) c3(protein eyes shut homolog isoform 3 precursor) c4( )c5(259545, 259546, 259547, 259548, 259549, 259550, 259551, 259552, 259553, 259554) c6( )c7(IR, bpi, nw, nk, nC, ayj, ad); #c1(EZH1) c2(NP_001982) c3(histone-lysine N-methyltransferase EZH1) c4( ) c5(122833, 122834, 122835, 122836, 122837, 122838, 122839, 122840, 122841, 122842, 122843) c6( )c7(hy, ne, BN, as); #c1(EZH1) c2(XP_005257201) c3(histone-lysine N-methyltransferase EZH1 isoform X1) c4(274740, 274741, 274742) c5(274743, 274744, 274745, 274746, 274747, 274748) c6( )c7(hy, ne, BN, as); #c1(EZH1) c2(XP_005257202) c3(histone-lysine N-methyltransferase EZH1 isoform X4) c4(274749) c5(274750) c6( )c7(hy, ne, BN, as); #c1(EZH1) c2(XP_005257203) c3(histone-lysine N-methyltransferase EZH1 isoform X5) c4( )c5(274751, 274752, 274753, 274754, 274755) c6( )c7(hy, ne, BN, as); #c1(EZH2) c2(XP_005250019) c3(histone-lysine N-methyltransferase EZH2 isoform X3) c4(270001) c5(270002, 270003, 270004, 270005, 270006, 270007) c6( )c7(ID, gh, T, aC, if, fd, bB, ci, oB, bv, aE, fm, aDZ, MO, pk, eL, ly, oP, aW, uY, g, as, eF, cm, bw, iP, yd, qJ, eM, nB, iA, iT, U, cj, iS, dW, k, m, ajm, Or, n, aj, fr, qN, ft, jz, TK, gd, kH, h, dL, ey, dx, RT, MD, PX, aq, kp, yq, cM, op, lx, YE, bP, dB, hL, pV, pg, fL, ph, nz, it, B, fM, dA, aA, aYh, q, J, qH, er, eA, eJ, XX, nv, aT, rO, bq, ae, af, jf, il, Y, r, qL, en, t, f, ik, ck, aRp, iM, kP, ac, RW, np, bt, LS, id, iW, DA, jh, hF, Fc, iJ, mC, ad, hd, py, io, hP, d, je, vj, ff, eZ, af, iR, bz, LB, bE, bC, y); #c1(EZH2) c2(XP_005250020) c3(histone-lysine N-methyltransferase EZH2 isoform X5) c4(270008) c5(270009, 270010, 270011, 270012, 270013, 270014) c6( )c7(ID, gh, T, aC, if, fd, bB, ci, oB, bv, aE, fm, aDZ, MO, pk, eL, ly, oP, aW, uY, g, as, eF, cm, bw, iP, yd, qJ, eM, nB, iA, iT, U, cj, iS, dW, k, m, ajm, Or, n, aj, fr, qN, ft, jz, TK, gd, kH, h, dL, ey, dx, RT, MD, PX, aq, kp, yq, cM, op, Ix, YE, bP, dB, hL, pV, pg, fL, ph, nz, it, B, fM, dA, aA, aYh, q, J, qH, er, eA, eJ, XX, nv, aT, rO, bq, ae, af, jf, il, Y, r, qL, en, t, f, ik, ck, aRp, iM, kP, ac, RW, np, bt, LS, id, iW, oA, jh, hF, Fc, iJ, mC, ad, hd, py, io, hP, d, je, vj, ff, eZ, af, iR, bz, LB, bE, bC, y); #c1(EZH2) c2(XP_005250021) c3(histone-lysine N-methyltransferase EZH2 isoform X15) c4(270015, 270016) c5(270017, 270018, 270019, 270020, 270021, 270022, 270023, 270024, 270025, 270026) c6( )c7(ID, gh, T, aC, if, fd, bB, ci, oB, bv, aE, fm, aDZ, MO, pk, eL, ly, oP, aW, uY, g, as, eF, cm, bw, iP, yd, qJ, eM, nB, iA, iT, U, cj, iS, dW, k, m, ajm, Or, n, aj, fr, qN, ft, jz, TK, gd, kH, h, dL, ey, dx, RT, MD, PX, aq, kp, yq, cM, op, lx, YE, bP, dB, hL, pV, pg, fL, ph, nz, it, B, fM, dA, aA, aYh, q, J, qH, er, eA, eJ, XX, nv, aT, rO, bq, ae, af, jf, il, Y, r, qL, en, t, f, ik, ck, aRp, iM, kP, ac, RW, np, bt, LS, id, iW, oA, jh, hF, Fc, iJ, mC, ad, hd, py, io, hP, d, je, vj, ff, eZ, aF, iR, bz, LB, bE, bC, y); #c1(EZH2) c2(XP_011514185) c3(histone-lysine N-methyltransferase EZH2 isoform X2) c4( )c5(306796) c6( )c7(ID, gh, T, aC, if, fd, bB, ci, oB, bv, aE, fm, aDZ, MO, pk, eL, ly, oP, aW, uY, g, as, eF, cm, bw, iP, yd, qJ, eM, nB, iA, iT, U, cj, iS, dW, k, m, ajm, Or, n, aj, fr, qN, ft, jz, TK, gd, kH, h, dL, ey, dx, RT, MD, PX, aq, kp, yq, cM, op, Ix, YE, bP, dB, hL, pV, pg, fL, ph, nz, it, B, fM, dA, aA, aYh, q, J, qH, er, eA, eJ, XX, nv, aT, rO, bq, ae, af, jf, iU, Y, r, qL, en, t, f, ik, ck, aRp, iM, kP, ac, RW, np, bt, LS, id, iW, oA, jh, hF, Fc, iJ, mC, ad, hd, py, io, hP, d, je, vj, ff, eZ, af, iR, bz, LB, bE, bC, y); #c1(EZH2) c2(XP_011514186) c3(histone-lysine N-methyltransferase EZH2 isoform X1) c4(306797) c5(306798, 306799, 306800, 306801, 306802) c6( )c7(ID, gh, T, aC, if, fd, bB, ci, oB, bv, aE, fm, aDZ, MO, pk, eL, ly, oP, aW, uY, g, as, eF, cm, bw, iP, yd, qJ, eM, nB, iA, iT, U, cj, iS, dW, k, m, ajm, Or, n, aj, fr, qN, ft, jz, TK, gd, kH, h, dL, ey, dx, RT, MD, PX, aq, kp, yq, cM, op, lx, YE, bP, dB, hL, pV, pg, fL, ph, nz, it, B, fM, dA, aA, aYh, q, J, qH, er, eA, eJ, XX, nv, aT, rO, bq, ae, af, jf, iU, Y, r, qL, en, t, f, ik, ck, aRp, iM, kP, ac, RW, np, bt, LS, id, iW, DA, jh, hF, Fc, iJ, mC, ad, hd, py, io, hP, d, je, vj, ff, eZ, aF, iR, bz, LB, bE, bC, y); #c1(EZH2) c2(XP_011514188) c3(histone-lysine N-methyltransferase EZH2 isoform X6) c4( )c5(306803) c6( )c7(ID, gh, T, aC, if, fd, bB, ci, oB, bv, aE, fm, aDZ, MO, pk, eL, ly, oP, aW, uY, g, as, eF, cm, bw, iP, yd, qJ, eM, nB, iA, iT, U, cj, iS, dW, k, m, ajm, Or, n, aj, fr, qN, ft, jz, TK, gd, kH, h, dL, ey, dx, RT, MD, PX, aq, kp, yq, cM, op, lx, YE, bP, dB, hL, pV, pg, fL, ph, nz, it, B, fM, dA, aA, aYh, q, J, qH, er, eA, eJ, XX, nv, aT, rO, bq, ae, af, jf, iU, Y, r, qL, en, t, f, ik, ck, aRp, iM, kP, ac, RW, np, bt, LS, id, iW, oA, jh, hF, Fc, iJ, mC, ad, hd, py, io, hP, d, je, vj, ff, eZ, af, iR, bz, LB, bE, bC, y); #c1 (EZH2) c2(XP_011514195) c3(histone-lysine N-methyltransferase EZH2 isoform X12) c4(306804) c5(306805, 306806, 306807, 306808, 306809, 306810) c6( )c7(ID, gh, T, aC, if, fd, bB, ci, oB, bv, aE, fm, aDZ, MO, pk, eL, ly, oP, aW, uY, g, as, eF, cm, bw, iP, yd, qJ, eM, nB, iA, iT, U, cj, iS, dW, k, m, ajm, Or, n, aj, fr, qN, ft, jz, TK, gd, kH, h, dL, ey, dx, RT, MD, PX, aq, kp, yq, cM, op, lx, YE, bP, dB, hL, pV, pg, fL, ph, nz, it, B, fM, dA, aA, aYh, q, J, qH, er, eA, eJ, XX, nv, aT, rO, bq, ae, af, jf, iU, Y, r, qL, en, t, f, ik, ck, aRp, iM, kP, ac, RW, np, bt, LS, id, iW, oA, jh, hF, Fc, iJ, mC, ad, hd, py, io, hP, d, je, vj, ff, eZ, aF, iR, bz, LB, bE, bC, y); #c1(EZH2) c2(XP_011514204) c3(histone-lysine N-methyltransferase EZH2 isoform X20) c4( )c5(306811, 306812, 306813, 306814, 306815) c6( ) c7(ID, gh, T, aC, if, fd, bB, ci, oB, bv, aE, fm, aDZ, MO, pk, eL, ly, oP, aW, uY, g, as, eF, cm, bw, iP, yd, qJ, eM, nB, iA, iT, U, cj, iS, dW, k, m, ajm, Or, n, aj, fr, qN, ft, jz, TK, gd, kH, h, dL, ey, dx, RT, MD, PX, aq, kp, yq, cM, op, lx, YE, bP, dB, hL, pV, pg, fL, ph, nz, it, B, fM, dA, aA, aYh, q, J, qH, er, eA, eJ, XX, nv, aT, rO, bq, ae, af, jf, il, Y, r, qL, en, t, f, ik, ck, aRp, iM, kP, ac, RW, np, bt, LS, id, iW, DA, jh, hF, Fc, iJ, mC, ad, hd, py, io, hP, d, je, vj, ff, eZ, aF, iR, bz, LB, bE, bC, y); #c1(EZR) c2(NP_001104547) c3(ezrin) c4(42161, 42162) c5(42163, 42164, 42165, 42166, 42167, 42168, 42169, 42170, 42171, 42172, 42173, 42174, 42175, 42176, 42177, 42178) c6( )c7(bM, T, aC, lk, fR, hV, sw, fG, if, aA, ajp, bB, BF, ci, qN, fr, zP, q, ad, eA, ip, dP, bv, av, iu, aE, bpj, bW, cE, aT, h, By, dl, ey, K, hu, fb, ae, jf, zl, d, aW, Ls, md, aFK, eH, r, hQ, Ej, ff, aF, en, cl, eF, t, cM, bz, RV, W, eM, Ew, bE, X, hL, at, m, gn, qF, nz, it, B); #c1(F10) c2(NP_000495) c3 (coagulation factor X preproprotein) c4(9935) c5(9936, 9937, 9938, 9939, 9940, 9941, 9942, 9943, 9944, 9945, 9946, 9947, 9948, 9949, 9950, 9951, 9952, 9953, 9954, 9955, 9956, 9957, 9958, 9959, 9960, 9961, 9962, 9963, 9964, 9965, 9966, 9967, 9968, 9969, 9970, 9971, 9972, 9973, 9974, 9975, 9976, 9977, 9978, 9979, 9980, 9981, 9982, 9983, 9984, 9985, 9986, 9987, 9988, 9989, 9990, 9991, 9992, 9993, 9994, 9995, 9996, 9997, 9998, 9999, 10000, 10001, 10002, 10003, 10004, 10005, 10006, 10007, 10008, 10009, 10010, 10011, 10012, 10013, 10014, 10015, 10016, 10017, 10018, 10019, 10020, 10021, 10022, 10023, 10024, 10025, 10026, 10027, 10028, 10029, 10030, 10031, 10032, 10033, 10034, 10035, 10036, 10037, 10038, 10039, 10040, 10041, 10042, 10043, 10044, 10045, 10046, 10047, 10048, 10049, 10050, 10051, 10052, 10053, 10054, 10055, 10056, 10057, 10058, 10059, 10060, 10061, 10062, 10063, 10064, 10065, 10066, 10067, 10068, 10069, 10070, 10071, 10072, 10073, 10074, 10075, 10076, 10077, 10078, 10079, 10080, 10081, 10082, 10083) c6(10084, 10085) c7(bM, T, jv, bpn, fR, e, aw, bpk, j, bpl, eA, UE, aE, Ot, qh, dV, aT, h, af, hm, arl, bpm, lE, Y, aaD, aUF, ac, dX, dy); #c1(F11) c2(NP_000119) c3(coagulation factor XI precursor) c4(1965, 1966, 1967) c5(1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081) c6(2082, 2083, 2084, 2085, 2086, 2087, 2088) c7(P, qk, wA, A, ag, bpp, M, cz, dX, j, bpo, Pd, be, bpq, uC, ae, ad); #c1(F11R) c2(NP_058642) c3(junctional adhesion molecule A precursor) c4(192913, 192914, 192915) c5(192916, 192917, 192918, 192919, 192920, 192921, 192922, 192923, 192924, 192925, 192926, 192927, 192928, 192929, 192930, 192931, 192932, 192933, 192934, 192935, 192936, 192937, 192938, 192939, 192940, 192941) c6( )c7(bL, T, dD, ea, atj, cz, v, j, eq, eA, av, db, MI, aT, cy, ji, K, bq, BO, qt, aW, Y, fe, vs, af, iV, t, ac, eM, Fi, dh); #c1(F12) c2(NP_000496) c3(coagulation factor X11 precursor) c4(10086) c5(10087, 10088, 10089, 10090, 10091, 10092, 10093, 10094, 10095, 10096, 10097, 10098, 10099, 10100, 10101, 10102, 10103, 10104, 10105, 10106, 10107, 10108, 10109, 10110, 10111, 10112, 10113, 10114, 10115, 10116, 10117, 10118, 10119, 10120, 10121, 10122, 10123, 10124, 10125, 10126, 10127, 10128, 10129, 10130, 10131, 10132, 10133, 10134, 10135, 10136, 10137, 10138, 10139, 10140, 10141, 10142, 10143, 10144, 10145, 10146, 10147, 10148, 10149, 10150, 10151, 10152, 10153, 10154, 10155, 10156, 10157, 10158, 10159, 10160, 10161, 10162, 10163, 10164, 10165, 10166, 10167, 10168, 10169, 10170, 10171, 10172, 10173, 10174, 10175, 10176, 10177, 10178, 10179, 10180) c6(10181) c7(bL, sY, bpt, eR, jp, e, aw, cz, j, vh, vC, M, ey, bpr, cH, bps, dN, YG, uC, cM, el, ac, ag, RJ, qq); #c1(F13A1) c2(NP_000120) c3(coagulation factor XIII A chain precursor) c4(2089, 2090, 2091) c5(2092, 2093, 2094, 2095, 2096, 2097, 2098, 2099, 2100, 2101, 2102, 2103, 2104, 2105, 2106, 2107, 2108, 2109, 2110, 2111, 2112, 2113, 2114, 2115, 2116, 2117, 2118, 2119, 2120, 2121, 2122, 2123, 2124, 2125, 2126, 2127, 2128, 2129, 2130, 2131, 2132, 2133, 2134, 2135, 2136, 2137, 2138, 2139, 2140, 2141, 2142, 2143, 2144, 2145, 2146, 2147, 2148, 2149, 2150, 2151, 2152, 2153, 2154, 2155, 2156, 2157, 2158, 2159, 2160, 2161, 2162, 2163, 2164, 2165, 2166, 2167, 2168, 2169, 2170, 2171, 2172, 2173, 2174, 2175, 2176, 2177, 2178, 2179, 2180, 2181, 2182, 2183, 2184, 2185, 2186, 2187, 2188, 2189, 2190, 2191, 2192, 2193, 2194, 2195, 2196) c6(2197) c7(azs, sY, dD, ahf, aaJ, qb, ea, wL, e, aw, DV, bpu, cz, vr, ez, aj, v, j, eq, C, ad, ip, Na, vh, bpy, M, Nc, jj, dQ, ae, af, aiJ, eu, fJ, cr, yy, fe, yq, cM, aVc, bz, bpx, uC, adA, DT, Cq, apM, W, ac, kB, bpv, vb, U, qq, sU, tz, dX, aS, k, m, pw, GF, y, wx, us, bpw); #c1(F13A1) c2(XP_011512644) c3(coagulation factor XIII A chain isoform X2) c4(303625) c5(303626, 303627) c6( )c7(azs, sY, dD, ahf, aaJ, qb, ea, wL, e, aw, DV, bpu, cz, vr, ez, aj, v, j, eq, C, ad, ip, Na, vh, bpy, M, Nc, jj, dQ, ae, af, aiJ, eu, fJ, cr, yy, fe, yq, cM, aVc, bz, bpx, uC, adA, DT, Cq, apM, W, ac, kB, bpv, vb, U, qq, sU, tz, dX, aS, k, m, pw, GF, y, wx, us, bpw); #c1(F13B) c2(NP_001985) c3(coagulation factor XIII B chain precursor) c4(122851) c5(122852, 122853, 122854, 122855, 122856, 122857) c6( )c7(sY, dD, aaJ, aw, vr, j, eq, ad, bpz, jj, ae, eu, aiJ, jf, r, fe, nk, uC, bz, W, ac, U, aS, bpy); #c1(F2) c2(NP_000497) c3(prothrombin preproprotein) c4(10182, 10183) c5(10184, 10185, 10186, 10187, 10188, 10189, 10190, 10191, 10192, 10193, 10194, 10195, 10196, 10197, 10198, 10199, 10200, 10201, 10202, 10203, 10204, 10205, 10206, 10207, 10208, 10209, 10210, 10211, 10212, 10213, 10214, 10215, 10216, 10217, 10218, 10219, 10220, 10221, 10222, 10223, 10224, 10225, 10226, 10227, 10228, 10229, 10230, 10231, 10232, 10233, 10234, 10235, 10236, 10237, 10238, 10239, 10240, 10241, 10242, 10243, 10244, 10245, 10246, 10247, 10248, 10249, 10250, 10251, 10252, 10253, 10254, 10255, 10256, 10257, 10258, 10259, 10260, 10261, 10262, 10263, 10264, 10265, 10266, 10267, 10268, 10269, 10270, 10271, 10272, 10273, 10274, 10275, 10276, 10277, 10278, 10279, 10280, 10281, 10282, 10283, 10284, 10285, 10286, 10287, 10288) c6(10289, 10290, 10291, 10292, 10293) c7(bpX, bpG, T, ahf, qo, fR, aSz, UH, ea, e, al, l, eb, aiC, ame, ur, ss, aR, Hr, x, bpl, xE, vh, M, JT, Yk, K, dQ, hH, eN, aiJ, hm, aa, bpU, DA, bpR, bpL, LC, aW, ih, hl, fc, bpP, YG, vs, aUG, bpM, aaD, aVc, uC, ve, wE, aBO, Gs, mx, uo, sl, wx, mk, k, m, pw, Ro, P, bpH, dD, Rx, aal, s, xu, aw, O, ez, n, BH, aj, fr, Mo, ec, bpN, vC, ql, bpF, bpk, lG, aal, DU, aBR, aWY, ux, is, Gc, Vb, hi, OV, MX, sq, QM, vu, Ff, ge, fe, aq, aUP, pm, cM, bpD, vU, iO, kT, W, bpC, se, afc, aoB, qq, oh, ut, gv, fl, va, bD, B, bL, bpS, jv, eR, AL, mr, wL, fG, DV, aA, sM, cz, vv, v, j, C, aqf, bpW, q, J, eA, oT, ga, hb, nX, rS, sl, HC, bpJ, aT, cy, dz, rz, qk, af, eu, kl, arl, Y, r, iy, aPf, en, pb, el, sn, c, ac, WP, mG, bpB, tz, bpE, bpl, dy, jQ, vi, gx, sY, uq, qb, dM, ha, Jy, vr, oR, aET, bpT, uu, jE, eq, gb, atK, mC, hX, dP, F, oz, vJ, bpp, qh, qV, lC, LA, dm, lQ, jo, adx, sj, bpm, azS, Di, bpV, cH, cW, ff, wc, bpO, eZ, yy, bpA, aF, Pd, iV, dr, ek, gk, DT, pc, pE, ag, gZ, MJ, dX, aS, ei, oQ, GF, hE, ra, sH); #c1(F2R) c2(NP_001983) c3(proteinase-activated receptor 1 precursor) c4(122844) c5(122845, 122846, (22847, 122848, 122849, 122850) c6( )c7(ID, bM, G, T, aC, fR, Js, pf, ea, e, al, bO, bB, ci, bv, aE, K, cp, aa, mH, aW, az, fc, Qm, hJ, eM, bpY, iA, acS, cj, uJ, mk, m, hh, cE, dD, aw, n, fr, ct, rc, jz, Ha, jK, OV, anP, auM, fe, aq, jq, KT, Qu, kz, vb, bo, hL, at, aiS, B, Jo, fG, jx, aA, cz, j, C, q, eA, ady, aT, cy, an, ae, af, eu, nO, oV, Y, lo, HL, en, cl, iK, pb, ck, ov, ac, fD, bt, WV, bS, eq, ad, BY, dP, av, HF, F, vJ, ds, nJ, d, ue, cW, ff, eZ, aF, Z, bz, bdu, ek, LB, pv, EW, es, bE, uR, lF, Hs, dX, MC, y, X); #c1(F2RL1) c2(NP_005233) c3(proteinase-activated receptor 2 precursor) c4(154273) c5(154274, 154275, 154276, 154277, 154278, 154279) c6( )c7(oO, bM, T, lD, aC, ea, if, bB, ci, fw, aE, rw, aa, aW, md, zD, Qq, g, eE, zV, ba, Hb, m, aCS, cE, aZN, dD, aw, ez, fr, qN, cd, ai, ke, jz, h, sp, Ni, fe, hk, hL, JM, at, uz, zB, B, fG, aA, v, j, V, q, eA, aT, cy, ae, Y, bg, r, lo, en, t, cT, EY, fD, bt, ao, Mb, eq, ad, BY, dP, av, sN, ds, ji, d, ff, cr, dN, gl, aF, iV, DF, LB, zr, xZ, bE, X, lC, ra, iu, y); #c1(F2RL2) c2(NP_001243495) c3 (proteinase-activated receptor 3 isoform 2) c4( )c5(90937, 90938, 90939, 90940, 90941) c6( )c7(LB, bv, s, fG, aT, fr, ci, hE, it, B); #c1(F2RL2) c2(NP_004092) c3(proteinase-activated receptor 3 isoform 1 precursor) c4(143504, 143505) c5(143506, 143507, 143508, 143509, 143510, 143511, 143512) c6( )c7(LB, bv, s, fG, aT, fr, ci, hE, it, B); #c1(F2RL3) c2(NP_003941) c3(proteinase-activated receptor 4 precursor) c4(142346) c5(142347, 142348, 142349, 142350, 142351, 142352) c6( )c7(gh, T, aC, G, bS, j, ct, eA, ad, av, jz, aT, h, K, he, ae, af, CB, jf, Uf, B, aW, Y, auM, eH, bpZ, cl, Z, hJ, eF, bz, cm, ek, ck, ac, wl, acS, U, hL, lF, dX, EY, ra, X); #c1(F5) c2(NP_000121) c3(coagulation factor V precursor) c4(2198, 2199, 2200) c5(2201, 2202, 2203, 2204, 2205, 2206, 2207, 2208, 2209, 2210, 2211, 2212, 2213, 2214, 2215, 2216, 2217, 2218, 2219, 2220, 2221, 2222, 2223, 2224, 2225, 2226, 2227, 2228, 2229, 2230, 2231, 2232, 2233, 2234, 2235, 2236, 2237, 2238, 2239, 2240, 2241, 2242, 2243, 2244, 2245, 2246, 2247, 2248, 2249, 2250, 2251, 2252, 2253, 2254, 2255, 2256, 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 2290, 2291, 2292, 2293, 2294, 2295, 2296, 2297, 2298, 2299, 2300, 2301, 2302, 2303, 2304, 2305, 2306, 2307, 2308, 2309, 2310, 2311, 2312, 2313, 2314, 2315, 2316, 2317, 2318, 2319, 2320, 2321, 2322, 2323, 2324, 2325, 2326, 2327, 2328, 2329, 2330, 2331, 2332, 2333, 2334, 2335, 2336, 2337, 2338, 2339, 2340, 2341, 2342, 2343, 2344, 2345, 2346, 2347, 2348, 2349, 2350, 2351, 2352, 2353, 2354, 2355, 2356, 2357, 2358) c6( )c7(ID, bpG, T, ahf, qo, fR, aSz, UH, ea, e, al, eb, aiC, fd, ame, ur, Lw, aY, ss, aR, Hr, bpl, dY, vh, M, JT, Vv, us, dQ, hH, eN, aiJ, hm, bpU, bpR, aW, ih, hl, YG, vs, aUG, g, bpM, uC, aVc, aHQ, ve, wE, aBO, bqd, mx, Gs, uo, sU, wx, bqh, k, m, pw, sr, P, bqn, FW, bpH, dD, aaJ, sQ, xu, aw, fN, O, ez, n, aj, oY, bqq, ld, aSv, b, vC, ql, i, bqp, aal, DU, wb, aWY, ux, aBR, is, Gc, Vb, bqb, hi, OV, MX, sq, vu, Ff, ge, fQ, fe, aUP, pm, cM, sE, vU, ar, kT, bqk, W, bpC, se, aoB, qq, oh, ut, gv, fl, bqo, aHP, bqg, bD, B, lp, bL, ach, jv, bpS, eR, AL, mr, wL, dk, De, DV, aA, sM, cz, fF, Cg, j, v, C, aqf, vv, bpW, q, J, UE, oT, ga, DK, hb, nX, sl, MP, aT, cy, dz, nl, rz, bqe, qk, eu, bA, kl, arl, r, iy, lo, en, pb, el, BU, wg, sn, c, ac, mG, DW, gX, bpB, tz, cS, bpE, bpl, dy, bqa, ao, gx, sY, bof, uq, qb, aZ, dM, ha, Jy, vr, OR, bqr, uu, ah, eq, gb, mC, bqs, BY, F, vJ, qh, qV, IC, LA, dm, bql, ji, lQ, jo, adx, sj, azS, ue, bpV, cH, bqc, ff, wc, bpO, eZ, yy, hv, bpA, Pd, iV, aUC, dr, ek, bqj, gk, DT, pc, pE, ag, sG, gZ, bqi, MJ, dX, aLA, aS, ei, Hs, GF, bqm, vt, X, sH); #c1(F7) c2(NP_000122) c3(coagulation factor V11 isoform a preproprotein) c4(2359) c5(2360, 2361, 2362, 2363, 2364, 2365, 2366, 2367, 2368, 2369, 2370, 2371, 2372, 2373, 2374, 2375, 2376, 2377, 2378, 2379, 2380, 2381, 2382, 2383, 2384, 2385, 2386, 2387, 2388, 2389, 2390, 2391, 2392, 2393, 2394, 2395, 2396, 2397, 2398, 2399, 2400, 2401, 2402, 2403, 2404, 2405, 2406, 2407, 2408, 2409, 2410, 2411, 2412, 2413, 2414, 2415, 2416, 2417, 2418, 2419, 2420, 2421, 2422, 2423, 2424, 2425, 2426, 2427, 2428, 2429, 2430, 2431, 2432, 2433, 2434, 2435, 2436, 2437, 2438, 2439, 2440, 2441, 2442, 2443, 2444, 2445, 2446, 2447, 2448, 2449, 2450, 2451, 2452, 2453, 2454, 2455) c6(2456, 2457, 2458) c7(T, FR, ea, e, al, bB, GK, M, dQ, eL, eN, hm, bqt, aW, hC, hl, aaD, ahv, DL, uC, wE, uo, m, aUL, dD, sl, aw, jA, aj, TB, acb, bpq, MQ, bqy, aq, cM, bJ, kT, bqx, qq, ut, B, bL, dA, eR, mr, wL, De, bqv, DV, cz, sM, bqu, v, j, C, q, eA, ga, aT, cy, bq, qk, af, eu, bg, r, iy, t, f, ck, ac, c, cS, dy, gT, sY, qb, vr, oR, bpT, Sv, uu, eq, ad, qh, ZU, lQ, bqw, uX, bpm, bpV, cW, dq, Pd, ek, gk, bE, ag, MJ, dX, aS, GF); #c1(F7) c2(NP_001254483) c3(coagulation factor V11 isoform c preproprotein) c4(94444) c5(94445, 94446, 94447, 94448, 94449, 94450, 94451, 94452, 94453) c6(94454, 94455) c7(T, fR, ea, e, al, bB, GK, M, dQ, eL, eN, hm, bqt, aW, hC, hl, aaD, ahv, DL, uC, wE, uo, m, aUL, dD, sQ, aw, jA, aj, TB, acb, bpq, MQ, bqy, aq, cM, bJ, kT, bqx, qq, ut, B, bL, dA, eR, mr, wL, oe, bqv, DV, cz, sM, bqu, v, j, C, q, eA, ga, aT, cy, bq, qk, af, eu, bg, r, iy, t, f, ck, ac, c, cS, dy, gT, sY, qb, vr, oR, bpT, Sv, uu, eq, ad, qh, ZU, IQ, bqw, uX, bpm, bpV, cW, dq, Pd, ek, gk, bE, ag, MJ, dX, aS, GF); #c1(F7) c2(NP_062562) c3(coagulation factor V11 isoform b preproprotein) c4(201965) c5(201966, 201967, 201968, 201969, 201970, 201971, 201972, 201973, 201974) c6(201975, 201976) c7(T, FR, ea, e, al, bB, GK, M, dQ, eL, eN, hm, bqt, aW, hC, hl, aaD, ahv, DL, uC, wE, uo, m, aUL, dD, sQ, aw, jA, aj, TB, acb, bpq, MQ, bqy, aq, cM, bJ, kT, bqx, qq, ut, B, bL, dA, eR, mr, wl, oe, bqv, DV, cz, sM, bqu, v, j, C, q, eA, ga, aT, cy, bq, qk, af, eu, bg, r, iy, t, f, ck, ac, c, cS, dy, gT, sY, qb, vr, OR, bpT, Sv, uu, eq, ad, qh, ZU, lQ, bqw, uX, bpm, bpV, cW, dq, Pd, ek, gk, bE, ag, MJ, dX, aS, GF); #c1(F8) c2(NP_000123) c3(coagulation factor VIII isoform a precursor) c4(2459, 2460) c5(2461, 2462, 2463, 2464, 2465, 2466, 2467, 2468, 2469, 2470) c6(2471, 2472, 2473) c7(T, dA, pA, aU, qb, aw, al, cz, sM, n, vv, bpT, j, aiC, bqA, aSw, ib, rw, qh, M, aT, acb, h, ux, aUH, qk, Ck, bqB, aeT, sq, xs, hl, aUD, YG, aMJ, Pd, aUC, bqz, DI, dr, bJ, hq, wg, sn, ac, aUJ, se, ag, wE, Ei, qq, dX, bqC, hE, ao, Ro); #c1(F8) c2(NP_063916) c3(coagulation factor VIII isoform b) c4(202215) c5(202216, 202217, 202218, 202219, 202220, 202221, 202222, 202223) c6(202224, 202225) c7(T, dA, pA, aUI, qb, aw, al, cz, sM, n, vv, bpT, j, aiC, bqA, aSw, ib, rw, qh, M, aT, acb, h, ux, aUH, qk, Ck, bqB, aeT, sq, xs, hl, aUD, YG, aMJ, Pd, aUC, bqz, DI, dr, bJ, hq, wg, sn, ac, aUJ, se, ag, wE, Ei, qq, dX, bqC, hE, ao, Ro); #c1(F9) c2(NP_000124) c3(coagulation factor 1X preproprotein) c4(2474, 2475) c5(2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2536, 2537, 2538, 2539, 2540, 2541, 2542, 2543, 2544, 2545, 2546, 2547, 2548, 2549, 2550, 2551, 2552, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2579, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 2606, 2607, 2608, 2609, 2610, 2611, 2612, 2613, 2614, 2615, 2616, 2617, 2618, 2619, 2620, 2621, 2622, 2623, 2624, 2625, 2626, 2627, 2628, 2629, 2630, 2631, 2632, 2633, 2634, 2635, 2636, 2637, 2638, 2639, 2640, 2641, 2642, 2643, 2644, 2645, 2646, 2647, 2648, 2649, 2650, 2651, 2652, 2653, 2654, 2655, 2656, 2657, 2658, 2659, 2660, 2661, 2662, 2663, 2664, 2665, 2666, 2667, 2668, 2669, 2670, 2671, 2672, 2673, 2674) c6(2675, 2676, 2677, 2678, 2679, 2680, 2681, 2682, 2683) c7(T, aCh, al, aiC, ame, ci, aon, xY, Vp, xy, bqD, vh, rw, K, bqE, hH, eL, aCg, aW, aoo, hl, YG, Ku, ir, bw, aaD, aAW, qJ, Jr, nB, U, wx, qR, P, NK, aw, n, nA, aCi, azj, kH, h, mY, bpq, Lg, wC, sq, agG, aq, aMJ, bP, se, hk, eD, qq, oh, YH, AN, B, AS, rG, dA, hz, V, nv, Qg, aT, Fx, bK, qk, jf, oV, r, lo, Vk, ck, sS, nM, iM, c, il, ao, Dd, Hn, bpT, ah, hF, hd, db, qh, io, pq, je, xs, cr, nf, Pd, dr, aJx, ag, dX, bcc, hE, y); #c1(F9) c2(XP_005262454) c3(coagulation factor 1X isoform X1) c4(277929) c5( )c6( )c7(T, aCh, al, aiC, ame, ci, aon, xY, Vp, xy, bqD, vh, rw, K, bqE, hH, eL, aCg, aW, aoo, hl, YG, Ku, ir, bw, aaD, aAW, qJ, Jr, nB, U, wx, qR, P, NK, aw, n, nA, aCi, azj, kH, h, mY, bpq, Lg, wC, sq, agG, aq, aMJ, bP, se, hk, eD, qq, oh, YH, AN, B, AS, rG, dA, hz, V, nv, Qg, aT, Fx, bk, qk, jf, oV, r, lo, Vk, ck, sS, nM, iM, c, il, ao, Dd, Hn, bpT, ah, hF, hd, db, qh, io, pq, je, xs, cr, nf, Pd, dr, alx, ag, dX, bcc, hE, y); #c1(FA2H) c2(NP_077282) c3(fatty acid 2-hydroxylase) c4(214900, 214901) c5(214902, 214903, 214904, 214905, 214906, 214907) c6( )c7(aPR, bO, v, Vs, ad, axa, cc, aV, iX, aT, aU, bqF, bK, K, cb, bqG, ae, af, aHE, Pw, aW, aAm, Mz, hp, jb, bN, ac, eM, U); #c1(FA2H) c2(XP_011521621) c3(fatty acid 2-hydroxylase isoform X3) c4( )c5(316419, 316420, 316421, 316422, 316423, 316424) c6( )c7(aPR, b, v, Vs, ad, axa, cc, aV, iX, aT, aU, bqF, bK, K, cb, bqG, ae, af, aHE, Pw, aW, aAm, Mz, hp, jb, bN, ac, eM, U); #c1(FAAH2) c2(NP_777572) c3(fatty-acid amide hydrolase 2) c4(25/164) c5(251165, 251166, 251167, 251168, 251169, 251170) c6( )c7(fr); #c1(FABP1) c2(NP_001434) c3(fatty acid-binding protein, liver) c4(117812) c5(117813, 117814, 117815, 117816, 117817, 117818) c6( )c7(bL, fU, D, eR, e, fc, oW, n, aET, dG, v, ey, ei, du, B, ek); #c1(FABP2) c2(NP_000125) c3(fatty acid-binding protein, intestinal) c4(2684) c5(2685, 2686, 2687, 2688, 2689, 2690) c6( )c7(bL, sY, dD, Ht, eR, ea, e, aw, cz, aj, v, j, eq, nS, q, J, kL, GK, td, bv, nH, Mm, ec, jy, qM, wt, dQ, eu, A, cH, fc, r, dq, iV, uC, fq, ek, bJ, kT, ac, cO, Fi, mg, mf, cS, dy, ei); #c1(FABP4) c2(NP_001433) c3(fatty acid-binding protein, adipocyte) c4(117805) c5(117806, 117807, 117808, 117809, 117810, 117811) c6( )c7(bL, bM, T, dD, id, ajN, aZ, ea, e, qQ, aj, v, j, ci, eq, zP, J, ad, M, aT, Vv, LA, h, dL, K, bq, ae, eL, jf, kl, zQ, cH, aW, t, uC, GR, baa, cS, m, ei); #c1(FABP5) c2(NP_001435) c3(fatty acid-binding protein, epidermal) c4(117819) c5(117820, 117821, 117822, 117823, 117824, 117825) c6( )c7(kV, oO, G, bL, yG, n, v, j, ci, ad, aT, bK, aiJ, yF, aW, ff, cr, kp, en, kP, W, hL, at, fD, y, B); #c1(FABP6) c2(NP_001124430) c3(gastrotropin isoform 1) c4(48832, 48833) c5(48834, 48835, 48836, 48837, 48838, 48839, 48840, 48841, 48842, 48843) c6( )c7(bL, T, l, s, u, pf, wz, r, v, q); #c1(FABP6) c2(XP_006714893) c3(gastrotropin isoform X1) c4( )c5(288302, 288303, 288304, 288305, 288306, 288307) c6( )c7(bL, T, l, s, u, pf, wz, r, v, q); #c1(FABP7) c2(NP_001437) c3(fatty acid-binding protein, brain) c4(117826) c5(117827, 117828, 117829, 117830, 117831, 117832) c6( )c7(ha, n, bS, fr, ct, V, eA, aR, aeO, aT, bk, hP, atX, d, aW, Y, fQ, as, Z, f, cm, bN, Hp, Dy, iA, dB); #c1(FABP7) c2(XP_005266915) c3(fatty acid-binding protein, brain isoform X1) c4(280397) c5(280398, 280399, 280400, 280401, 280402, 280403) c6( )c7(ha, n, bS, fr, ct, V, eA, aR, aeO, aT, bK, hP, atX, d, aW, Y, fl, as, Z, f, cm, bN, Hp, Dy, iA, dB); #c1(FABP9) c2(NP_001073995) c3(fatty acid-binding protein 9) c4(36847) c5(36848, 36849, 36850, 36851, 36852, 36853) c6( )c7(H, eZ); #c1(FADD) c2(NP_003815) c3(FAS-associated death domain protein) c4(141097) c5(141098, 141099, 141100, 141101, 141102, 141103) c6( )c7(T, dA, fR, aA, fd, oY, aj, aDO, fr, ci, Qp, eA, J, ad, eW, bv, av, bql, akJ, oW, aT, h, an, ae, eG, A, d, eH, Az, eZ, yq, g, eF, mK, kn, bqH, bP, dB, mG, gX, cj, aS, m, y, bt, it, B); #c1(FADS1) c2(NP_037534) c3(fatty acid desaturase 1) c4(176075, 176076, 176077) c5(176078, 176079, 176080, 176081, 176082, 176083) c6( )c7(bL, bM, T, e, EQ, aA, cz, aj, bS, j, ct, v, J, aqN, afR, nX, M, bq, Z, W, Fi, Zw); #c1(FADS1) c2(XP_011543324) c3(fatty acid desaturase 1 isoform X1) c4( )c5(342106, 342107, 342108, 342109, 342110, 342111, 342112, 342113, 342114, 342115, 342116, 342117, 342118, 342119, 342120, 342121, 342122, 342123, 342124, 342125) c6( )c7(bL, bM, T, e, EQ, aA, cz, aj, bS, j, ct, v, J, aqN, afR, nX, M, bq, Z, W, Fi, Zw); #c1(FADS2) c2(NP_001268430) c3(fatty acid desaturase 2 isoform 2 precursor) c4(99923) c5(99924, 99925, 99926, 99927, 99928, 99929) c6( )c7(bL, bM, T, W, nX, e, aw, aT, h, aj, bS, j, ct, bK, du, Z, v, ei, J, wD, aqN); #c1(FADS2) c2(NP_004256) c3(fatty acid desaturase 2 isoform 1) c4(144639, 144640) c5(144641, 144642, 144643, 144644, 144645, 144646, 144647, 144648, 144649, 144650, 144651, 144652) c6( )c7(bL, bM, T, W, nX, e, aw, aT, h, aj, bS, j, ct, bK, du, Z, v, ei, J, wD, aqN); #c1(FADS3) c2(NP_068373) c3(fatty acid desaturase 3) c4(209360) c5(209361, 209362, 209363, 209364, 209365, 209366, 209367, 209368, 209369, 209370, 209371, 209372) c6( )c7(bL, c, fm, nX, bci, j, bqJ); #c1(FADS3) c2(XP_011543325) c3(fatty acid desaturase 3 isoform X1) c4(342126) c5( )c6( )c7(bL, c, fm, nX, bci, j, bqJ); #c1(FAF2) c2(NP_055428) c3(FAS-associated factor 2) c4(181077, 181078) c5(181079, 181080, 181081, 181082, 181083, 181084) c6( )c7(ad); #c1(FAF2) c2(XP_011532778) c3(FAS-associated factor 2 isoform X2) c4( )c5(328859, 328860, 328861, 328862, 328863, 328864) c6( )c7(ad); #c1(FAH) c2(NP_000128) c3(fumarylacetoacetase) c4(2698, 2699, 2700, 2701, 2702, 2703, 2704) c5(2705, 2706, 2707, 2708, 2709, 2710, 2711, 2712, 2713, 2714, 2715, 2716, 2717, 2718, 2719, 2720, 2721, 2722, 2723, 2724, 2725, 2726, 2727, 2728, 2729, 2730, 2731, 2732, 2733, 2734, 2735, 2736, 2737, 2738, 2739, 2740, 2741, 2742, 2743, 2744, 2745, 2746, 2747, 2748, 2749, 2750, 2751, 2752, 2753, 2754, 2755, 2756, 2757, 2758, 2759, 2760, 2761, 2762, 2763, 2764, 2765, 2766, 2767, 2768, 2769, 2770, 2771, 2772, 2773, 2774, 2775, 2776, 2777, 2778, 2779, 2780, 2781, 2782, 2783, 2784, 2785, 2786, 2787, 2788, 2789, 2790, 2791, 2792, 2793, 2794, 2795, 2796, 2797, 2798, 2799, 2800, 2801, 2802, 2803, 2804, 2805, 2806, 2807, 2808, 2809, 2810, 2811, 2812, 2813, 2814, 2815, 2816, 2817, 2818, 2819, 2820, 2821, 2822, 2823, 2824, 2825, 2826, 2827, 2828, 2829, 2830, 2831, 2832, 2833, 2834, 2835, 2836, 2837, 2838, 2839, 2840, 2841, 2842, 2843, 2844, 2845, 2846, 2847, 2848, 2849, 2850, 2851, 2852, 2853, 2854, 2855, 2856, 2857) c6(2858) c7(os, oe, al, cz, aj, oZ, ci, q, bqK, aT, K, Gy, aW, hl, r, dq, lo, aq, iR, dr, gk, kQ, QT, B); #c1(FAHD2A) c2(NP_057128) c3(fumarylacetoacetate hydrolase domain-containing protein 2A) c4(189028) c5(189029, 189030, 189031, 189032, 189033, 189034, 189035, 189036, 189037, 189038) c6( )c7(v); #c1(FAHD2A) c2(XP_011509589) c3(fumarylacetoacetate hydrolase domain-containing protein 2A isoform X2) c4(300154) c5( )c6( )c7(v); #c1(FAHD2A) c2(XP_011509592) c3(fumarylacetoacetate hydrolase domain-containing protein 2A isoform X4) c4(300155) c5(300156, 300157, 300158, 300159, 300160, 300161, 300162, 300163, 300164, 300165, 300166, 300167) c6( )c7(v); #c1(FAIM2) c2(NP_036438) c3(protein lifeguard 2) c4(173856, 173857) c5(173858, 173859, 173860, 173861, 173862, 173863, 173864, 173865, 173866, 173867, 173868, 173869, 173870, 173871, 173872, 173873) c6( )c7(bL, aw, cz, jy, v, fq); #c1(FAIM3) c2(NP_001135945) c3(fas apoptotic inhibitory molecule 3 isoform b) c4(53677) c5(53678, 53679, 53680, 53681, 53682, 53683) c6( )c7(cj); #c1(FAIM3) c2(NP_001180267) c3(fas apoptotic inhibitory molecule 3 isoform c precursor) c4(73602) c5(73603, 73604, 73605, 73606, 73607, 73608, 73609, 73610, 73611, 73612, 73613, 73614, 73615, 73616, 73617, 73618, 73619, 73620, 73621, 73622, 73623, 73624, 73625, 73626) c6( )c7(cj); #c1(FAIM3) c2(NP_005440) c3(fas apoptotic inhibitory molecule 3 isoform a precursor) c4(156191, 156192) c5(156193, 156194, 156195, 156196, 156197, 156198) c6( )c7(cj); #c1(FAIM3) c2(XP_005273408) c3(fas apoptotic inhibitory molecule 3 isoform X1) c4(284577, 284578) c5( ) c6( )c7(cj); #c1(FAM10ZA) c2(NP_001030331) c3(protein FAM102A isoform a) c4(31012, 31013) c5(31014, 31015, 31016, 31017, 31018, 31019) c6( )c7(aW, lH, aT); #c1(FAM102A) c2(NP_976050) c3(protein FAM102A isoform b) c4( )c5(262899, 262900, 262901, 262902, 262903) c6( )c7(aW, lH, aT); #c1(FAM102A) c2(XP_011516980) c3(protein FAM10ZA isoform X3) c4( )c5(310479) c6( )c7(aW, lH, aT); #c1(FAM103AI) c2(NP_113640) c3(RNMT-activating mini protein) c4(221870) c5(221871, 221872, 221873, 221874, 221875, 221876) c6( ) c7(ck, dE, y); #c1(FAM102A) c2(NP_061891) c3(inactive ubiquitin thioesterase FAM105A) c4(201002) c5(201003, 201004, 201005, 201006, 201007, 201008) c6( )c7(ey); #c1(FAM107A) c2(NP_001269642) c3(protein FAM107A isoform b) c4(102362) c5(102363, 102364, 102365, 102366, 102367, 102368, 102369, 102370) c6( )c7(fr, T, d, eA, y); #c1(FAM107A) c2(NP_001269643) c3(protein FAM107A isoform c) c4(102371, 102372, 102373, 102374) c5(102375, 102376, 102377, 102378, 102379, 102380, 102381, 102382) c6( )c7(fr, T, d, eA, y); #c1 (FAM107A) c2(NP_009108) c3(protein FAM107A isoform a) c4( )c5(170707, 170708, 170709, 170710, 170711, 170712, 170713, 170714, 170715, 170716, 170717, 170718, 170719, 170720, 170721, 170722, 170723, 170724) c6( )c7(fr, T, d, eA, y); #c1(FAM107B) c2(NP_001269625) c3(protein FAM107B isoform a) c4( )c5(102239, 102240, 102241, 102242, 102243, 102244, 102245, 102246, 102247, 102248, 102249, 102250, 102251, 102252, 102253, 102254, 102255, 102256, 102257, 102258, 102259, 102260, 102261, 102262, 102263, 102264, 102265, 102266, 102267, 102268, 102269, 102270, 102271, 102272, 102273, 102274, 102275, 102276, 102277, 102278, 102279, 102280, 102281, 102282, 102283, 102284, 102285, 102286, 102287, 102288, 102289, 102290, 102291, 102292, 102293, 102294, 102295, 102296, 102297, 102298, 102299, 102300, 102301, 102302, 102303, 102304, 102305, 102306, 102307, 102308, 102309, 102310, 102311, 102312, 102313) c6( )c7(T, oe, gX); #c1(FAM107B) c2(NP_113641) c3(protein FAM107B isoform b) c4(221877, 221878, 221879, 221880, 221881, 221882, 221883, 221884, 221885, 221886) c5(221887, 221888, 221889, 221890, 221891, 221892) c6( )c7(T, oe, gX); #c1(FAM107B) c2(XP_011518033) c3(protein FAM107B isoform X2) c4( )c5(312033, 312034, 312035, 312036, 312037, 312038, 312039, 312040, 312041, 312042, 312043, 312044, 312045, 312046, 312047, 312048, 312049, 312050, 312051) c6( )c7(T, oe, gX); #c1(FAM109A) c2(NP_001171467) c3(sesquipedalian-1 isoform 1) c4(70153, 70154, 70155, 70156, 70157, 70158) c5(70159, 70160, 70161, 70162, 70163, 70164, 70165, 70166, 70167, 70168, 70169, 70/70, 70171, 70172) c6( )c7(mU, aPt, mW, mZ, KW, mS, mY, bK, mV, mT, mX); #c1(FAM109A) c2(NP_001171468) c3(sesquipedalian-1 isoform 2) c4( )c5(70173, 70174, 70175, 70176, 70177, 70178, 70179, 70180, 70181, 70182, 70183, 70184, 70185, 70186, 70187, 70188, 70189, 70190, 70191, 70192, 70193, 70194, 70195, 70196, 70197, 70198, 70199, 70200, 70201, 70202, 70203, 70204, 70205, 70206, 70207, 70208) c6( )c7(mU, aPt, mW, mZ, KW, mS, mY, bK, mV, mT, mX); #c1(FAM110A) c2(NP_001276075) c3(protein FAM110A) c4(107865) c5(107866, 107867, 107868, 107869, 107870, 107871, 107872, 107873, 107874, 107875, 107876, 107877, 107878, 107879, 107880, 107881, 107882, 107883, 107884, 107885, 107886, 107887, 107888, 107889) c6( )c7(dX, bpm, eA, aUF); #c1(FAM110B) c2(NP_67 (722) c3(protein FAM110B) c4(242160) c5(242161, 242162, 242163, 242164, 242165, 242166) c6( )c7(ae, np, ad); #c1(FAM110C) c2(NP_001071178) c3(protein FAM110C) c4(35346) c5(35347, 35348, 35349, 35350, 35351, 35352) c6( )c7(aaw); #c1(FAM111B) c2(NP_001136/76) c3(protein FAM111B isoform b) c4( )c5(54500, 54501, 54502, 54503, 54504, 54505, 54506, 54507, 54508, 54509, 54510, 54511, 54512) c6( )c7(bqM, fG, fr, bql, ask); #c1(FAM111B) c2(NP_945185) c3(protein FAM111B isoform a) c4(260704, 260705, 260706) c5(260707, 260708, 260709, 260710, 260711, 260712, 260713, 260714, 260715, 260716) c6( )c7(bqM, fG, fr, bql, ask); #c1(FAM114A1) c2(NP_612398) c3(protein NOXP20) c4(236377, 236378) c5(236379, 236380, 236381, 236382, 236383, 236384) c6( ) c7(cm); #c1(FAM114A1) c2(XP_005262730) c3(protein NOXP20 isoform X3) c4( )c5(278076, 278077, 278078, 278079, 278080, 278081) c6( )c7(cm); #c1(FAM114A1) c2(XP_006714096) c3(protein NOXP20 isoform X4) c4( )c5(287541, 287542, 287543, 287544, 287545, 287546) c6( )c7(cm); #c1(FAM114A1) c2(XP_011512065) c3(protein NOXP20 isoform X2) c4(302720) c5( )c6( )c7(cm); #c1(FAM120B) c2(NP_001273308) c3(constitutive coactivator of peroxisome proliferator-activated receptor gamma isoform a) c4(104663, 104664, 104665) c5(104666, 104667, 104668, 104669, 104670, 104671, 104672, 104673, 104674, 104675, 104676, 104677) c6( )c7(adz); #c1(FAM120B) c2(NP_001273309) c3(constitutive coactivator of peroxisome proliferator-activated receptor gamma isoform b) c4(104678) c5(104679, 104680, 104681, 104682, 104683, 104684) c6( )c7(adz); #c1(FAM120B) c2(NP_001273310) c3(constitutive coactivator of peroxisome proliferator-activated receptor gamma isoform d) c4( )c5(104685, 104686, 104687, 104688, 104689) c6( ) c7(adz); #c1(FAM120B) c2(NP_115824) c3(constitutive coactivator of peroxisome proliferator-activated receptor gamma isoform c) c4( )c5(224164) c6( )c7(adz); #c1(FAM120C) c2(NP_060318) c3(constitutive coactivator of PPAR-gamma-like protein 2 isoform 1) c4(195388, 195389) c5(195390, 195391, 195392, 195393, 195394, 195395) c6( )c7(nf, bN, bqN); #c1(FAM120C) c2(NP_940858) c3(constitutive coactivator of PPAR-gamma-like protein 2 isoform 2) c4( )c5(259977, 259978, 259979, 259980, 259981, 259982) c6( )c7(nf, bN, bqN); #c1(FAM124B) c2(NP_001116251) c3(protein FAM124B isoform a) c4(43718) c5(43719, 43720, 43721, 43722, 43723, 43724) c6( )c7(aw, aT); #c1(FAM124B) c2(NP_079061) c3(protein FAM124B isoform b) c4(216841) c5(216842, 216843, 216844, 216845, 216846, 216847, 216848, 216849, 216850, 216851) c6( )c7(aw, aT); #c1(FAM126A) c2(NP_115970) c3(hyccin) c4(224993) c5(224994, 224995, 224996, 224997, 224998, 224999) c6( )c7(Kd, T, am, aC, bqP, Dd, uG, al, jx, aA, bB, n, jE, nA, ci, bX, q, aEl, hX, b, F, aE, lv, aT, yu, bK, K, aYF, pl, aAA, s, aW, Y, fc, hl, r, yy, aq, lo, aF, bqO, iR, boD, KM, dr, YR, ck, iO, gk, bP, bE, X, rH, cP, be, i, hE, lB, B, vi); #c1(FAM129A) c2(NP_443198) c3(protein Niban) c4(229815, 229816) c5(229817, 229818, 229819, 229820, 229821, 229822, 229823, 229824, 229825, 229826, 229827, 229828) c6( )c7(qJ, es, ci, MU); #c1(FAM129B) c2(NP_001030611) c3(niban-like protein 1 isoform 2) c4(31064) c5(31065, 31066, 31067, 31068, 31069, 31070, 31071, 31072, 31073, 31074, 31075, 31076) c6( )c7(Y); #c1(FAM129B) c2(NP_073744) c3(niban-like protein 1 isoform 1) c4(213433) c5(213434, 213435, 213436, 213437, 213438, 213439) c6( )c7(Y); #c1(FAM129B) c2(XP_005252192) c3(niban-like protein 1 isoform X1) c4(271593) c5( )c6( )c7(Y); #c1(FAM134B) c2(NP_001030022) c3 (protein FAM134B isoform 1) c4(30874) c5(30875, 30876, 30877, 30878, 30879, 30880) c6( )c7(bqR, cZ, T, s, RD, bE, aFa, Dd, ff, r, bB, bql, ci, en, dd, g, q, akS); #c1(FAM134B) c2(NP_061873) c3(protein FAM134B isoform 2) c4(200867) c5(200868, 200869, 200870, 200871, 200872, 200873) c6( )c7(bqR, cZ, T, s, RD, bE, aFa, Dd, ff, r, bB, bql, ci, en, dd, g, q, akS); #c1(FAM134B) c2(XP_011512355) c3 (protein FAM134B isoform X1) c4( )c5(303063, 303064, 303065, 303066, 303067, 303068) c6( )c7(bqR, cZ, T, s, RD, bE, aFa, Dd, ff, r, bB, bql, ci, en, dd, g, q, akS); #c1(FAM135A) c2(NP_001099001) c3(protein FAM135A isoform a) c4(41596) c5(41597, 41598, 41599, 41600, 41601, 41602) c6( )c7(bL, y, A, j, cz); #c1(FAM135A) c2(NP_001156001) c3(protein FAM135A isoform c) c4(61689, 61690, 61691) c5(61692, 61693, 61694, 61695, 61696, 61697, 61698, 61699, 61700, 61701, 61702, 61703) c6( )c7(bL, y, A, j, cz); #c1(FAM135A) c2(NP_065870) c3 (protein FAM135A isoform b) c4(205964) c5(205965, 205966, 205967, 205968, 205969, 205970, 205971, 205972, 205973, 205974) c6( ) c7(bL, y, A, j, cz); #c1(FAM135A) c2(XP_005248808) c3(protein FAM135A isoform X6) c4(268584) c5(268585, 268586) c6( )c7(bL, y, A, j, cz); #c1(FAM135A) c2(XP_011534298) c3(protein FAM135A isoform X2) c4( )c5(330671, 330672, 330673, 330674, 330675, 330676, 330677, 330678, 330679, 330680) c6( )c7(bL, y, A, j, cz); #c1(FAM135B) c2(NP_056996) c3(protein FAM135B) c4(188383) c5(188384, 188385, 188386, 188387, 188388, 188389) c6( )c7(en, T, ff, cd, bA); #c1(FAM135B) c2(XP_011515372) c3(protein FAM135B isoform X8) c4(308202, 308203) c5( )c6( )c7(en, T, ff, cd, bA); #c1(FAM135B) c2(XP_011515373) c3(protein FAM135B isoform X9) c4( )c5(308204, 308205, 308206, 308207, 308208, 308209, 308210, 308211, 308212, 308213, 308214, 308215, 308216, 308217) c6(308218, 308219) c7(en, T, ff, cd, bA); #c1(FAM136A) c2(NP_116211) c3(protein FAM136A) c4(225759) c5(225760, 225761, 225762, 225763, 225764, 225765, 225766, 225767) c6(225768, 225769) c7(Fh); #c1(FAM13A) c2(NP_001015045) c3(protein FAM13A isoform b) c4(26135) c5(26136, 26137, 26138, 26139, 26140, 26141) c6( )c7(LB, ant, ac, oe, la, at, dy, aa); #c1(FAM13A) c2(NP_001252508) c3(protein FAM13A isoform d) c4(94035) c5(94036, 94037, 94038, 94039, 94040, 94041) c6( )c7(LB, ant, ac, oe, la, at, dy, aa); #c1(FAM13A) c2(NP_055698) c3(protein FAM13A isoform a) c4(182854) c5(182855, 182856, 182857, 182858, 182859, 182860) c6( )c7(LB, ant, ac, oe, la, at, dy, aa); #c1(FAM13A) c2(XP_005262740) c3(protein FAM13A isoform X5) c4( )c5(278082, 278083, 278084, 278085, 278086, 278087) c6( )c7(LB, ant, ac, oe, la, at, dy, aa); #c1(FAM13A) c2(XP_011529819) c3(protein FAM13A isoform X4) c4(325628) c5( )c6( ) c7(LB, ant, ac, oe, la, at, dy, aa); #c1(FAM13C) c2(NP_001001971) c3(protein FAM13C isoform 2) c4(19223) c5(19224, 19225, 19226, 19227, 19228, 19229) c6( )c7(e, ac); #c1(FAM13C) c2(NP_001137245) c3(protein FAM13C isoform 3) c4(55160) c5( )c6( )c7(e, ac); #c1(FAM13C) c2(NP_001160170) c3(protein FAM13C isoform 4) c4(65195) c5(65196, 65197, 65198, 65199, 65200) c6( )c7(e, ac); #c1(FAM13C) c2(NP_937858) c3(protein FAM13C isoform 1) c4(259315, 259316) c5(259317, 259318, 259319, 259320, 259321, 259322, 259323, 259324, 259325, 259326, 259327, 259328, 259329, 259330, 259331, 259332, 259333) c6( )c7(e, ac); #c1(FAM13C) c2(XP_005269675) c3(protein FAM13C isoform X2) c4(282703) c5(282704, 282705, 282706, 282707, 282708, 282709, 282710) c6( ) c7(e, ac); #c1(FAM13C) c2(XP_005269676) c3(protein FAM13C isoform X4) c4( )c5(282711, 282712) c6( )c7(e, ac); #c1(FAM149A) c2(NP_056213) c3(protein FAM149A) c4(186420, 186421, 186422) c5(186423, 186424, 186425, 186426, 186427, 186428, 186429, 186430, 186431, 186432, 186433, 186434, 186435, 186436, 186437, 186438, 186439, 186440, 186441, 186442) c6( )c7(aC); #c1(FAM149A) c2(XP_011530139) c3(protein FAM149A isoform X5) c4( )c5(326037, 326038, 326039, 326040, 326041, 326042) c6( )c7(aC); #c1 (FAM150B) c2(NP_001002919) c3(protein FAM150B precursor) c4(19777, 19778) c5(19779, 19780, 19781, 19782, 19783, 19784, 19785, 19786, 19787, 19788, 19789, 19790, 19791, 19792, 19793) c6(19794, 19795, 19796) c7(fq); #c1(FAM155A) c2(NP_001073865) c3(transmembrane protein FAM155A) c4(36175) c5(36176, 36177, 36178, 36179, 36180, 36181) c6( )c7(ac, pN, iX, aw, oe, alv, cd); #c1(FAM160B1) c2(NP_001128523) c3(protein FAM160B1 isoform b) c4(50153) c5(50154, 50155, 50156, 50157, 50158, 50159, 50160, 50161, 50162, 50163, 50164, 50165) c6( )c7(Y); #c1(FAM160BI) c2(NP_065991) c3(protein FAM160B1 isoform a) c4(206612, 206613, 206614) c5(206615, 206616, 206617, 206618, 206619, 206620, 206621, 206622, 206623, 206624, 206625, 206626) c6( )c7(Y); #c1(FAM16IA) c2(NP_001188472) c3(protein FAM161A isoform 1) c4(79204) c5(79205, 79206, 79207, 79208, 79209, 79210) c6( )c7(oh, nC, nk); #c1(FAM16IA) c2(NP_115556) c3(protein FAM16IA isoform 2) c4(223154) c5(223155, 223156, 223157, 223158, 223159, 223160) c6( )c7(oh, nC, nk); #c1(FAM163A) c2(NP_775780) c3(protein FAM163A) c4(250273) c5(250274, 250275, 250276, 250277, 250278, 250279) c6( )c7(cm, T); #c1(FAM163B) c2(NP_001073984) c3(protein FAM163B) c4(36776, 36777) c5(36778, 36779, 36780, 36781, 36782, 36783, 36784, 36785, 36786, 36787, 36788, 36789, 36790, 36791, 36792, 36793) c6( )c7(aj, ac); #c1(FAM167A) c2(NP_444509) c3(protein FAM167A) c4(230341) c5(230342, 230343, 230344, 230345, 230346, 230347) c6( )c7(Bv, A, fN, eZ, fL, pb, If, V, Pr, mC, y); #c1 (FAM168A) c2(NP_001272979) c3(protein FAM168A isoform 1) c4(104/27) c5(104128, 104129, 104130, 104131, 104132, 104133, 104134, 104135) c6( )c7(nz, ff, en, aA); #c1(FAM168A) c2(NP_001272980) c3(protein FAM168A isoform 3) c4(104136) c5(104137, 104138, 104139, 104140, 104141, 104142) c6( )c7(nz, ff, en, aA); #c1(FAM168A) c2(NP_055974) c3(protein FAM168A isoform 2) c4(184819) c5(184820, 184821, 184822, 184823, 184824, 184825) c6( )c7(nz, ff, en, aA); #c1(FAM168B) c2(NP_001009993) c3(myelin-associated neurite-outgrowth inhibitor) c4(23783, 23784) c5(23785, 23786, 23787, 23788, 23789, 23790, 23791, 23792, 23793, 23794, 23795, 23796, 23797, 23798, 23799, 23800) c6( )c7(xs); #c1(FAM168B) c2(XP_011508896) c3(myelin-associated neurite-outgrowth inhibitor isoform X2) c4( )c5(299475) c6( )c7(xs); #c1(FAM169B) c2(NP_872368) c3(protein FAM169B) c4(256259) c5(256260, 256261, 256262, 256263, 256264, 256265, 256266, 256267, 256268, 256269) c6( )c7(fq, bqS); #c1(FAM170A) c2(NP_001157463) c3 (protein FAM170A isoform 2) c4(62210, 62211) c5(62212, 62213, 62214, 62215, 62216, 62217, 62218, 62219, 62220, 62221, 62222, 62223) c6( )c7(cd); #c1(FAM170A) c2(NP_877438) c3(protein FAM170A isoform 1) c4(256820, 256821) c5(256822, 256823, 256824, 256825, 256826, 256827, 256828, 256829, 256830, 256831, 256832, 256833) c6( )c7(cd); #c1(FAM172A) c2(NP_001156889) c3(protein FAM172A isoform 2) c4( )c5(62008, 62009, 62010, 62011, 62012, 62013, 62014) c6(62015, 62016) c7(fq); #c1(FAM172A) c2(NP_001156890) c3(protein FAM172A isoform 3) c4( )c5(62017, 62018, 62019, 62020, 62021, 62022, 62023) c6(62024, 62025) c7(fq); #c1(FAM172A) c2(NP_114431) c3(protein FAM172A isoform 1 precursor) c4(222747, 222748) c5(222749, 222750, 222751, 222752, 222753, 222754, 222755, 222756, 222757) c6(222758, 222759) c7(fq); #c1(FAM172A) c2(XP_005272162) c3(protein FAM172A isoform X8) c4(283731) c5(283732) c6( )c7(fq); #c1(FAM(73B) c2(NP_954584) c3(protein FAM173B isoform 1) c4(261065) c5(261066, 261067, 261068, 261069, 261070, 261071) c6( )c7(xZ, cZ); #c1(FAM174A) c2(NP_940909) c3(membrane protein FAM174A precursor) c4( )c5(260097, 260098, 260099, 260100, 260101, 260102) c6( )c7(fq, ww); #c1(FAM174A) c2(XP_006714663) c3(membrane protein FAM174A isoform X1) c4(287979) c5( )c6( )c7(fq, ww); #c1(FAM175A) c2(NP_620775) c3(BRCA1-A complex subunit Abraxas) c4(238118, 238119, 238120) c5(238121, 238122, 238123, 238124, 238125, 238126, 238127, 238128) c6( )c7(aW, t, m, aT); #c1(FAM175A) c2(XP_005263337) c3(BRCA1-A complex subunit Abraxas isoform X1) c4( )c5(278434, 278435, 278436, 278437, 278438, 278439, 278440, 278441, 278442, 278443) c6( )c7(aW, t, m, aT); #c1(FAM175B) c2(NP_115558) c3(BRISC complex subunit Abro1) c4(223194) c5(223195, 223196, 223197, 223198, 223199, 223200) c6( )c7(aw, j, K); #c1(FAM177A1) c2(NP_001072987) c3(protein FAM177Al isoform 2) c4( )c5(35461, 35462, 35463, 35464, 35465, 35466, 35467, 35468, 35469, 35470, 35471, 35472, 35473) c6( )c7(cL); #c1(FAM177Al) c2(NP_775878) c3(protein FAM(77Al isoform 1 precursor) c4(250549, 250550, 250551) c5(250552, 250553, 250554, 250555, 250556, 250557, 250558, 250559, 250560) c6( )c7(c1); #c1(FAM177B) c2(NP_997351) c3(protein FAM177B) c4(265418, 265419, 265420) c5(265421, 265422, 265423, 265424, 265425, 265426, 265427, 265428, 265429, 265430, 265431, 265432, 265433, 265434, 265435, 265436, 265437, 265438, 265439, 265440, 265441, 265442) c6( )c7(W); #c1(FAM178A) c2(NP_001129595) c3(protein FAM178A isoform 2) c4(51955) c5(51956, 51957, 51958, 51959, 51960, 51961, 51962, 51963, 51964, 51965, 51966, 51967) c6( )c7(Ea); #c1(FAM178A) c2(NP_060591) c3(protein FAM178A isoform 1) c4(196712) c5(196713, 196714, 196715, 196716, 196717, 196718) c6( )c7(Ea); #c1(FAM178A) c2(XP_011538247) c3(protein FAM178A isoform X4) c4(335855) c5( )c6( )c7(Ea); #c1(FAM178B) c2(NP_001116118) c3(protein FAM178B isoform A) c4(43627, 43628) c5(43629, 43630, 43631, 43632, 43633, 43634, 43635, 43636, 43637, 43638, 43639, 43640, 43641, 43642, 43643, 43644, 43645, 43646) c6( )c7(fq); #c1(FAM178B) c2(NP_057574) c3(protein FAM178B isoform B) c4( )c5(191409, 191410, 191411, 191412, 191413, 191414) c6( )c7(fq); #c1(FAM178B) c2(XP_011509595) c3(protein FAM178B isoform X1) c4(300175, 300176) c5( )c6( ) c7(fq); #c1(FAM179B) c2(NP_055906) c3(protein FAM179B) c4(184389) c5(184390, 184391, 184392, 184393, 184394, 184395, 184396, 184397, 184398, 184399, 184400, 184401, 184402, 184403, 184404, 184405, 184406, 184407) c6( )c7(fe, T); #c1(FAM179B) c2(XP_011534873) c3(protein FAM179B isoform X2) c4(331413) c5( )c6( )c7(fe, T); #c1(FAM180A) c2(NP_995327) c3 (protein FAM180A {00886765] precursor) c4(263893) c5(263894, 263895, 263896, 263897, 263898, 263899, 263900, 263901, 263902, 263903, 263904, 263905, 263906, 263907, 263908, 263909, 263910, 263911, 263912) c6(263913, 263914, 263915, 263916, 263917) c7(dP); #c1(FAM184A) c2(NP_001093881) c3(protein FAM184A isoform 2) c4(40483) c5(40484, 40485, 40486, 40487, 40488) c6( )c7(IB); #c1(FAM184A) c2(NP_078857) c3(protein FAM184A isoform 1) c4(215804) c5(215805, 215806, 215807, 215808, 215809, 215810, 215811) c6( )c7(IB); #c1(FAM184B) c2(NP_056503) c3(protein FAM184B) c4(187798) c5(187799, 187800, 187801, 187802, 187803, 187804) c6( ) c7(IB); #c1(FAM187B) c2(NP_689694) c3(protein FAM187B precursor) c4(243588, 243589) c5(243590, 243591, 243592, 243593, 243594, 243595, 243596, 243597, 243598, 243599, 243600, 243601, 243602, 243603) c6( )c7(j); #c1(FAM188A) c2(NP_079224) c3(protein FAM188A) c4(217513) c5(217514, 217515, 217516, 217517, 217518, 217519) c6( )c7(bv, T, cc, eM, sN, mx, fe, v, nA, im, bz); #c1(FAM188A) c2(XP_006717571) c3(protein FAM188A isoform X2) c4(291180) c5( )c6( )c7(bv, T, cc, eM, sN, mx, fe, v, nA, im, bz); #c1(FAM188A) c2(XP_011517994) c3(protein FAM188A isoform X4) c4(312009) c5( )c6( )c7(bv, T, cc, eM, sN, mx, fe, v, nA, im, bz); #c1(FAM188A) c2(XP_011517995) c3(protein FAM188A isoform X5) c4(312010) c5(312011, 312012, 312013, 312014, 312015, 312016, 312017, 312018, 312019, 312020, 312021, 312022) c6( )c7(bv, T, cc, eM, sN, mx, fe, v, nA, im, bz); #c1(FAM188B) c2(NP_115598) c3(protein FAM188B) c4(223339) c5(223340, 223341, 223342, 223343, 223344, 223345, 223346) c6( )c7(aw); #c1(FAM189B) c2(NP_001254537) c3(protein FAM189B isoform c) c4(94606) c5(94607, 94608, 94609, 94610, 94611, 94612, 94613, 94614, 94615, 94616, 94617, 94618, 94619, 94620) c6( )c7(B); #c1(FAM189B) c2(NP_006580) c3(protein FAM189B isoform a) c4(166377) c5(166378, 166379, 166380, 166381, 166382, 166383, 166384, 166385) c6( )c7(B); #c1(FAM189B) c2(NP_937995) c3(protein FAM189B isoform b) c4(259465, 259466, 259467) c5(259468, 259469, 259470, 259471, 259472, 259473, 259474, 259475, 259476, 259477, 259478, 259479, 259480, 259481, 259482, 259483, 259484) c6( )c7(B); #c1(FAM189B) c2(XP_005276795) c3(protein FAM189B isoform X1) c4(285713) c5( )c6( ) c7(B); #c1(FAM189B) c2(XP_005276798) c3(protein FAM189B isoform X4) c4( )c5(285714, 285715, 285716, 285717, 285718, 285719, 285720) c6( )c7(B); #c1(FAM193B) c2(NP_001177875) c3(protein FAM193B) c4(73109) c5(73110, 73111, 73112, 73113, 73114, 73115) c6( ) c7(bv, Xu); #c1(FAM193B) c2(XP_006714938) c3(protein FAM193B isoform X1) c4( )c5(288365) c6( )c7(bv, Xu); #c1(FAM193B) c2(XP_006714942) c3(protein FAM193B isoform X7) c4(288366) c5(288367, 288368, 288369, 288370, 288371) c6( )c7(bv, Xu); #c1(FAM193B) c2(XP_006714948) c3(protein FAM193B isoform X9) c4( )c5(288372, 288373, 288374, 288375, 288376) c6( )c7(bv, Xu); #c1(FAM193B) c2(XP_006714949) c3(protein FAM193B isoform X10) c4( )c5(288377) c6( )c7(bv, Xu); #c1(FAM193B) c2(XP_011532880) c3(protein FAM193B isoform X5) c4(329069) c5( )c6( )c7(bv, Xu); #c1(FAM193B) c2(XP_011532883) c3(protein FAM193B isoform X8) c4( )c5(329070) c6( )c7(bv, Xu); #c1(FAM196A) c2(NP_001034851) c3(protein FAM196A) c4(32364) c5(32365, 32366, 32367, 32368, 32369, 32370) c6( )c7(aur, c); #c1(FAM196B) c2(NP_001123363) c3(protein FAM196B) c4(47061) c5(47062, 47063, 47064, 47065, 47066, 47067) c6( )c7(bM, aw, ac); #c1(FAM198B) c2(NP_001026870) c3(protein FAM198B isoform 1) c4(29729) c5(29730, 29731, 29732, 29733, 29734, 29735) c6( )c7(ey); #c1(FAM198B) c2(NP_001121896) c3(protein FAM198B isoform 2) c4(46258, 46259) c5(46260, 46261, 46262, 46263, 46264, 46265, 46266, 46267, 46268, 46269, 46270, 46271, 46272, 46273, 46274, 46275, 46276, 46277, 46278) c6( )c7(ey); #c1(FAM19AI) c2(NP_998774) c3(protein FAM19Al precursor) c4(265940) c5(265941, 265942, 265943, 265944, 265945, 265946, 265947, 265948, 265949, 265950, 265951, 265952, 265953, 265954) c6(265955, 265956, 265957, 265958) c7(bL, Be); #c1(FAM19A2) c2(NP_848634) c3(protein FAM19A2 precursor) c4(254030) c5(254031, 254032, 254033, 254034, 254035, 254036, 254037, 254038, 254039, 254040) c6( )c7(fq); #c1(FAM19A4) c2(NP_001005527) c3(protein FAM19A4 precursor) c4(21827) c5(21828, 21829, 21830, 21831, 21832, 21833, 21834, 21835, 21836, 21837, 21838, 21839, 21840, 21841, 21842) c6(21843, 21844, 21845) c7(ac, ci, aT); #c1(FAM19A4) c2(XP_011531674) c3(protein FAM19A4 isoform X1) c4(327792) c5( )c6( )c7(ac, ci, aT); #c1(FAM19A5) c2(NP_001076436) c3(protein FAM19A5 isoform 1) c4(37421) c5(37422, 37423, 37424, 37425, 37426, 37427, 37428, 37429) c6(37430, 37431) c7(aC, aw, c, pN); #c1(FAM19A5) c2(NP_056196) c3(protein FAM19A5 isoform 2 precursor) c4(186274) c5(186275, 186276, 186277, 186278, 186279, 186280, 186281, 186282) c6(186283, 186284) c7(aC, aw, c, pN); #c1(FAM204A) c2(NP_071346) c3 (protein FAM204A) c4( )c5(211114, 211115, 211116, 211117, 211118, 211119, 211120, 211121, 211122, 211123, 211124, 211125, 211126) c6( )c7(aC); #c1(FAM204A) c2(XP_005270081) c3(protein FAM204A isoform X2) c4(283105, 283106) c5(283107, 283108, 283109) c6( )c7(aC); #c1(FAM205A) c2(NP_001135389) c3(protein FAM205A) c4(52832, 52833, 52834) c5(52835, 52836, 52837, 52838, 52839, 52840, 52841, 52842, 52843, 52844, 52845, 52846, 52847, 52848, 52849, 52850, 52851, 52852) c6( )c7(y); #c1(FAM20A) c2(NP_001230675) c3(protein FAM20A isoform b) c4( )c5(86553, 86554, 86555, 86556, 86557, 86558) c6( )c7(bqT, asU, j, Ud); #c1(FAM20A) c2(NP_060035) c3(protein FAM20A isoform a precursor) c4(193806) c5(193807, 193808, 193809, 193810, 193811, 193812, 193813, 193814, 193815, 193816) c6( )c7(bqT, asU, j, Ud); #c1(FAM20A) c2(XP_011523219) c3(protein FAM20A isoform X2) c4(317756) c5( )c6( )c7(bqT, asU, j, Ud); #c1(FAM20B) c2(NP_055679) c3(glycosaminoglycan xylosylkinase) c4(182719, 182720) c5(182721, 182722, 182723, 182724, 182725, 182726, 182727, 182728, 182729, 182730, 182731, 182732) c6( )c7(fq, Ly); #c1(FAM20C) c2(NP_064608) c3(extracellular serine/threonine protein kinase FAM20C precursor) c4(203258) c5(203259, 203260, 203261, 203262, 203263, 203264) c6( )c7(Kd, bqU, AB, aDs, Hz, afS, Ay, zV, K, fr, PJ, bqV, ars, sH); #c1(FAM210B) c2(NP_543011) c3(protein FAM210B) c4(233236) c5(233237, 233238, 233239, 233240, 233241, 233242, 233243) c6( )c7(m); #c1(FAM213A) c2(NP_001230707) c3(redox-regulatory protein FAM213A isoform 1 precursor) c4(86645, 86646) c5(86647, 86648, 86649, 86650, 86651, 86652, 86653, 86654, 86655, 86656, 86657, 86658, 86659, 86660, 86661, 86662, 86663, 86664, 86665, 86666, 86667) c6(86668, 86669, 86670, 86671, 86672, 86673) c7(bqW); #c1(FAM213A) c2(NP_001230710) c3(redox-regulatory protein FAM213A isoform 2 precursor) c4( )c5(86674, 86675, 86676, 86677, 86678, 86679, 86680) c6(86681, 86682) c7(bqW); #c1(FAM214A) c2(NP_001273424) c3(protein FAM214A isoform 2) c4(104865) c5(104866, 104867, 104868, 104869, 104870, 104871) c6( )c7(ac); #c1(FAM214A) c2(NP_062546) c3(protein FAM214A isoform 1) c4(201915, 201916, 201917) c5(201918, 201919, 201920, 201921, 201922, 201923, 201924, 201925, 201926, 201927, 201928, 201929, 201930, 201931, 201932, 201933, 201934, 201935) c6( )c7(ac); #c1(FAM216A) c2(NP_037432) c3(protein FAM216A) c4(175515) c5(175516, 175517, 175518, 175519, 175520, 175521) c6( )c7(ey); #c1(FAM216A) c2(XP_005253932) c3(protein FAM216A isoform X2) c4( ) c5(272771) c6( )c7(ey); #c1(FAM220A) c2(NP_001032240) c3(protein FAM220A) c4(31124, 31125, 31126, 31127) c5(31128, 31129, 31130, 31131, 31132, 31133, 31134, 31135, 31136, 31137, 31138, 31139, 31140, 31141, 31142, 31143, 31144, 31145, 31146, 31147, 31148, 31149, 31150, 31151) c6( )c7(Y); #c1(FAM227B) c2(NP_689860) c3(protein FAM227B) c4(244188) c5(244189, 244190, 244191, 244192, 244193, 244194) c6( ) c7(Vp); #c1(FAM227B) c2(XP_005254273) c3(protein FAM2278 isoform X4) c4(273136, 273137, 273138) c5(273139) c6( )c7(Vp); #c1(FAM227B) c2(XP_011519627) c3(protein FAM227B isoform X14) c4( )c5(314300) c6( )c7(Vp); #c1(FAM227B) c2(XP_011519628) c3(protein FAM227B isoform X15) c4( )c5(314301, 314302, 314303, 314304, 314305, 314306, 314307, 314308, 314309, 314310, 314311, 314312, 314313, 314314, 314315, 314316, 314317) c6( )c7(Vp); #c1(FAM32A) c2(NP_054796) c3(protein FAM32A) c4(177986) c5(177987, 177988, 177989, 177990, 177991) c6( )c7(t, m, aT); #c1(FAM32A) c2(XP_011526200) c3(protein FAM32A isoform X1) c4( )c5(320471) c6( ) c7(t, m, aT); #c1(FAM3A) c2(NP_001164604) c3(protein FAM3A isoform 2) c4(67030) c5(67031, 67032, 67033, 67034, 67035, 67036, 67037, 67038) c6(67039, 67040) c7(bF, T, pa, eG, dA, baE, eM, bE, ev, X, gd, r, bB, dq, ey, ci, bz, q, adn); #c1(FAM3A) c2(NP_001164605) c3(protein FAM3A isoform 3) c4(67041) c5(67042, 67043, 67044, 67045, 67046, 67047, 67048, 67049) c6(67050, 67051) c7(bF, T, pa, eG, dA, baE, eM, bE, ev, X, gd, r, bB, dq, ey, ci, bz, q, adn); #c1(FAM3A) c2(NP_001269241) c3(protein FAM3A isoform 1 precursor) c4(100879) c5(100880, 100881, 100882, 100883, 100884, 100885, 100886, 100887, 100888, 100889, 100890, 100891, 100892, 100893, 100894, 100895, 100896, 100897, 100898, 100899, 100900, 100901, 100902, 100903) c6(100904, 100905, 100906, 100907, 100908, 100909) c7(bF, T, pa, eG, dA, baE, eM, bE, ev, X, gd, r, bB, dq, ey, ci, bz, q, adn); #c1(FAM3A) c2(XP_006724896) c3 (protein FAM3A isoform X6) c4( )c5(296492) c6( )c7(bF, T, pa, eG, dA, baE, eM, bE, ev, X, gd, r, bB, dq, ey, ci, bz, q, adn); #c1(FAM3B) c2(NP_478066) c3(protein FAM3B isoform a precursor) c4(230876) c5(230877, 230878, 230879, 230880, 230881, 230882, 230883, 230884) c6(230885, 230886) c7(T, dA, bE, fc, Pg, aA, bB, dG, xH, g, bD, bz, q); #c1(FAM3B) c2(NP_996847) c3(protein FAM3B isoform b) c4(264761) c5(264762, 264763, 264764, 264765, 264766, 264767, 264768, 264769) c6(264770, 264771) c7(T, dA, bE, fc, Pg, aA, bB, dG, xH, g, bD, bz, q); #c1(FAM3C) c2(NP_055703) c3(protein FAM3C precursor) c4(182893, 182894, 182895) c5(182896, 182897, 182898, 182899, 182900, 182901, 182902, 182903, 182904, 182905, 182906, 182907, 182908, 182909, 182910, 182911, 182912, 182913, 182914, 182915, 182916, 182917, 182918) c6(182919, 182920, 182921, 182922, 182923) c7(aE, v, agk, il, bz, B); #c1(FAM3D) c2(NP_620160) c3(protein FAM3D precursor) c4(237401, 237402) c5(237403, 237404, 237405, 237406, 237407, 237408, 237409, 237410, 237411, 237412, 237413, 237414, 237415, 237416, 237417, 237418) c6(237419, 237420, 237421, 237422) c7(cF); #c1(FAM46A) c2(NP_060103) c3(protein FAM46A) c4(194140, 194141, 194142, 194143, 194144) c5(194145, 194146, 194147, 194148, 194149, 194150, 194151, 194152, 194153, 194154, 194155, 194156, 194157, 194158, 194159, 194160, 194161, 194162, 194163, 194164, 194165, 194166, 194167, 194168, 194169, 194170, 194171, 194172, 194173, 194/74) c6( )c7(IR, aW, r, aT, j, nC, fq, q); #c1(FAM46D) c2(NP_689843) c3(protein FAM46D) c4(244131) c5(244132, 244133, 244134, 244135, 244136, 244137, 244138, 244139, 244140, 244141) c6( )c7(T, bu); #c1(FAM49A) c2(NP_110424) c3(protein FAM49A) c4(220269, 220270, 220271) c5(220272, 220273, 220274, 220275, 220276, 220277, 220278, 220279, 220280, 220281, 220282, 220283, 220284, 220285, 220286, 220287, 220288, 220289, 220290, 220291, 220292, 220293, 220294, 220295, 220296, 220297) c6( )c7(ey, cd, af, an); #c1(FAM49B) c2(NP_001243692) c3(protein FAM49B) c4(91413, 91414, 91415, 91416, 91417, 91418, 91419, 91420, 91421, 91422, 91423) c5(91424, 91425, 91426, 91427, 91428, 91429, 91430, 91431, 91432, 91433, 91434, 91435, 91436, 91437, 91438, 91439, 91440, 91441, 91442, 91443, 91444, 91445, 91446, 91447, 91448, 91449, 91450, 91451, 91452, 91453, 91454, 91455, 91456, 91457, 91458, 91459, 91460, 91461, 91462, 91463, 91464, 91465, 91466, 91467, 91468, 91469, 91470, 91471, 91472, 91473, 91474, 91475, 91476, 91477, 91478, 91479, 91480, 91481, 91482, 91483, 91484, 91485, 91486, 91487, 91488, 91489, 91490, 91491, 91492, 91493, 91494, 91495, 91496, 91497, 91498, 91499, 91500, 91501, 91502, 91503, 91504, 91505, 91506, 91507, 91508, 91509, 91510, 91511, 91512, 91513, 91514, 91515, 91516, 91517, 91518) c6( )c7(ey); #c1(FAM49B) c2(XP_011515409) c3(protein FAM49B isoform X2) c4( )c5(308236) c6( )c7(ey); #c1(FAM49B) c2(XP_011515422) c3 (protein FAM49B isoform X3) c4(308237) c5( )c6( )c7(ey); #c1(FAM50B) c2(NP_036267) c3(protein FAM50B) c4(172728) c5(172729, 172730, 172731, 172732, 172733, 172734, 172735, 172736, 172737, 172738) c6( )c7(VT); #c1(FAM53B) c2(NP_055476) c3 (protein FAM53B) c4(181348) c5(181349, 181350, 181351, 181352, 181353, 181354, 181355, 181356, 181357, 181358) c6( ) c7(fq, Gx); #c1(FAM57A) c2(NP_079068) c3(protein FAM57A) c4(216869, 216870) c5(216871, 216872, 216873, 216874, 216875, 216876, 216877, 216878, 216879, 216880, 216881, 216882) c6( )c7(av, T); #c1(FAM58A) c2(NP_001124469) c3(cyclin-related protein FAM58A isoform 2) c4(49108, 49/09) c5(49110, 49111, 49112, 49113, 49114, 49115, 49116, 49117, 49118, 49119, 49120, 49121) c6( )c7(qC, gh, ak, T, bqX, avH, aqp, dP, hK, aZ, baV, bO, cz, h, n, Rm, bS, bK, ct, Z); #c1(FAM58A) c2(NP_689487) c3(cyclin-related protein FAM58A isoform 1) c4(242848, 242849, 242850, 242851, 242852) c5(242853, 242854, 242855, 242856, 242857, 242858, 242859, 242860, 242861, 242862, 242863, 242864, 242865, 242866, 242867, 242868, 242869, 242870, 242871, 242872, 242873, 242874, 242875, 242876) c6( )c7(qC, gh, ak, T, bqX, avH, aqp, dP, hK, aZ, baV, bO, cz, h, n, Rm, bS, bK, ct, Z); #c1(FAM58A) c2(XP_005277978) c3(cyclin-related protein FAM58A isoform X2) c4(285751) c5(285752, 285753, 285754, 285755, 285756, 285757, 285758, 285759, 285760, 285761, 285762, 285763) c6( )c7(qC, gh, ak, T, bqX, avH, aqp, dP, hK, aZ, baV, bO, cz, h, n, Rm, bS, bK, ct, Z); #c1(FAM58A) c2(XP_011529517) c3(cyclin-related protein FAM58A isoform X3) c4(325047) c5(325048, 325049, 325050, 325051, 325052, 325053) c6( )c7(qC, gh, ak, T, bqX, avH, aqp, dP, hK, aZ, baV, bO, cz, h, n, Rm, bS, bk, ct, Z); #c1(FAM60A) c2(NP_001129284) c3(protein FAM60A) c4(51337) c5(51338, 51339, 51340, 51341, 51342, 51343, 51344, 51345, 51346, 51347, 51348, 51349, 51350, 51351, 51352, 51353, 51354, 51355, 51356, 51357, 51358, 51359, 51360, 51361) c6( )c7(Lf, bu); #c1(FAM60A) c2(XP_011544915) c3(protein FAM60A isoform X1) c4(343868, 343869, 343870, 343871) c5(343872, 343873, 343874, 343875, 343876, 343877, 343878, 343879, 343880, 343881, 343882) c6( )c7(Lf, bu); #c1(FAM63B) c2(NP_001035540) c3(protein FAM63B isoform a) c4(32894) c5(32895, 32896, 32897, 32898, 32899, 32900) c6( ) c7(aj, ac); #c1(FAM63B) c2(NP_001035543) c3(protein FAM63B isoform b) c4(32903) c5(32904, 32905, 32906, 32907, 32908, 32909, 32910) c6( )c7(aj, ac); #c1(FAM64A) c2(NP_001182157) c3(protein FAM64A isoform 2) c4(74631) c5(74632, 74633, 74634, 74635, 74636, 74637, 74638, 74639) c6( )c7(ck, aW, ey, kP, al); #c1(FAM64A) c2(NP_061886) c3(protein FAM64A isoform 1) c4(200981) c5(200982, 200983, 200984, 200985, 200986, 200987, 200988, 200989) c6( )c7(ck, aW, ey, kP, aT); #c1(FAM65B) c2(NP_001273374) c3(protein FAM65B isoform 3) c4(104791) c5(104792, 104793, 104794, 104795, 104796, 104797) c6( )c7(T, km, oj); #c1(FAM65B) c2(NP_001273375) c3(protein FAM65B isoform 4) c4(104798) c5(104799, 104800, 104801, 104802, 104803, 104804, 104805) c6( )c7(T, km, oj); #c1(FAM65B) c2(NP_001273376) c3(protein FAM65B isoform 5) c4(104806) c5(104807, 104808, 104809, 104810, 104811) c6( )c7(T, km, oj); #c1(FAM65B) c2(NP_055537) c3(protein FAM65B isoform 1) c4(181826) c5(181827, 181828, 181829, 181830, 181831) c6( )c7(T, km, oj); #c1(FAM65B) c2(NP_056948) c3(protein FAM65B isoform 2) c4( )c5(188171, 188172, 188173, 188174, 188175) c6( )c7(T, km, oj); #c1(FAM65B) c2(XP_006715344) c3(protein FAM65B isoform X5) c4( )c5(289497) c6( )c7(T, km, oj); #c1(FAM65B) c2(XP_011513312) c3(protein FAM65B isoform X3) c4(305586) c5( )c6( )c7(T, km, oj); #c1(FAM65B) c2(XP_011513314) c3(protein FAM65B isoform X6) c4( )c5(305587) c6( )c7(T, km, oj); #c1(FAM69C) c2(NP_001037834) c3(protein FAM69C) c4(34136) c5(34137, 34138, 34139, 34140, 34141, 34142) c6( )c7(fq); #c1(FAM71F1) c2(NP_115988) c3(protein FAM71F1 isoform 1) c4(225112) c5(225113, 225114, 225115, 225116, 225117, 225118) c6( )c7(v, ac); #c1(FAM71F2) c2(NP_001012457) c3(protein FAM71F2 isoform a) c4(25005) c5(25006, 25007, 25008, 25009, 25010, 25011) c6( )c7(wu, wm, bu); #c1(FAM71F2) c2(NP_001122398) c3(protein FAM71F2 isoform b) c4(46671) c5(46672, 46673, 46674, 46675, 46676, 46677, 46678, 46679, 46680, 46681, 46682, 46683, 46684, 46685) c6( ) c7(wu, wm, bu); #c1(FAM72B) c2(XP_011540322) c3(protein FAM72B isoform X3) c4(338742) c5(338743, 338744, 338745, 338746, 338747, 338748) c6( )c7(Ke, NL, fG, aA, h, aoG); #c1(FAM78B) c2(NP_001017961) c3(protein FAM78B) c4(26651) c5(26652, 26653, 26654, 26655, 26656, 26657, 26658, 26659, 26660, 26661) c6( )c7(bL, cm, bA, cz); #c1(FAM81B) c2(NP_689761) c3 (protein FAM81B) c4(243895) c5(243896, 243897, 243898, 243899, 243900, 243901) c6( )c7(cz); #c1(FAM83A) c2(NP_001275516) c3(protein FAM83A isoform c) c4(106781) c5(106782, 106783, 106784, 106785, 106786, 106787, 106788, 106789, 106790, 106791) c6( )c7(av, T, aW, aT, n, bz, iu); #c1(FAM83A) c2(NP_116288) c3(protein FAM83A isoform a) c4(226094) c5(226095, 226096, 226097, 226098, 226099, 226100, 226101, 226102, 226103, 226104) c6( )c7(av, T, aW, aT, n, bz, iu); #c1(FAM83A) c2(NP_996889) c3(protein FAM83A isoform b) c4(264815) c5(264816, 264817, 264818, 264819, 264820, 264821, 264822, 264823, 264824, 264825) c6( )c7(av, T, aW, aT, n, bz, iu); #c1(FAM83B) c2(NP_001010872) c3(protein FAM83B) c4(23884) c5(23885, 23886, 23887, 23888, 23889, 23890) c6( )c7(cm, aW, T, j, aT); #c1(FAM83D) c2(NP_112181) c3(protein FAM83D) c4(220653, 220654) c5(220655, 220656, 220657, 220658, 220659, 220660, 220661, 220662, 220663, 220664, 220665, 220666) c6( )c7(aW, aT); #c1(FAM83H) c2(XP_006725151) c3(protein FAM83H isoform X4) c4( )c5(296695) c6( )c7(Ud, bqY, q, Az, r); #c1(FAM83H) c2(XP_006725152) c3(protein FAM83H isoform X5) c4(296696) c5(296697, 296698, 296699, 296700, 296701) c6( )c7(Ud, bqY, q, Az, r); #c1(FAM84A) c2(NP_660158) c3(protein FAM84A) c4(240429, 240430, 240431) c5(240432, 240433, 240434, 240435, 240436, 240437, 240438, 240439, 240440, 240441, 240442, 240443, 240444, 240445, 240446, 240447, 240448, 240449, 240450, 240451, 240452, 240453) c6( )c7(bB, bE, Kc, kl, X); #c1(FAM84B) c2(NP_777571) c3(protein FAM84B) c4(251157) c5(251158, 251159, 251160, 251161, 251162, 251163) c6( )c7(t, T, aw, cd, ad); #c1(FAM89A) c2(NP_940954) c3(protein FAM89A) c4(260195) c5(260196, 260197, 260198, 260199, 260200, 260201) c6( )c7(AW); #c1(FAM92AI) c2(NP_001269963) c3 (protein FAM92Al isoform 2) c4(103295) c5(103296, 103297, 103298, 103299, 103300, 103301) c6( )c7(es, oe, fr, iA); #c1(FAM92A1) c2(NP_660312) c3(protein FAM92Al isoform 1) c4(240878, 240879, 240880) c5(240881, 240882, 240883, 240884, 240885, 240886, 240887, 240888, 240889, 240890, 240891, 240892, 240893, 240894, 240895, 240896, 240897, 240898) c6( )c7(es, oe, fr, iA); #c1(FAM92B) c2(NP_940893) c3(protein FAM92B) c4(260049, 260050) c5(260051, 260052, 260053, 260054, 260055, 260056, 260057, 260058, 260059, 260060, 260061, 260062) c6( )c7(fe, xN, iV); #c1(FAM9B) c2(NP_995321) c3(protein FAM9B) c4(263829, 263830) c5(263831, 263832, 263833, 263834, 263835, 263836, 263837, 263838, 263839, 263840) c6( )c7(c); #c1(FAM9B) c2(XP_011543767) c3(protein FAM9B isoform X2) c4( )c5(342796, 342797, 342798, 342799, 342800, 342801) c6( )c7(c); #c1(FAN1) c2(NP_001139568) c3(fanconi-associated nuclease 1 isoform b) c4(59490) c5(59491, 59492, 59493, 59494, 59495, 59496) c6( )c7(T, os, bqZ, aW, Y, aT, oZ, Hb, bra); #c1(FAN1) c2(NP_055782) c3(fanconi-associated nuclease 1 isoform a) c4(183470) c5(183471, 183472, 183473, 183474, 183475, 183476) c6( )c7(T, os, bqZ, aW, Y, aT, oZ, Hb, bra); #c1(FANCA) c2(NP_000126) c3(Fanconi anemia group A protein isoform a) c4(2691) c5(2692, 2693, 2694, 2695, 2696, 2697) c6( )c7(T, os, aC, QO, if, al, oY, oZ, qN, q, qK, aT, dL, K, eL, Gy, aW, ff, hl, r, dq, en, g, t, gk, kP, iT, QT, at, m, bt); #c1(FANCA) c2(NP_001018122) c3(Fanconi anemia group A protein isoform b) c4( )c5(27392, 27393, 27394, 27395, 27396, 27397, 27398, 27399, 27400, 27401, 27402, 27403, 27404, 27405, 27406, 27407, 27408, 27409) c6( ) c7(T, os, aC, Qo, if, al, oY, oZ, qN, q, qK, aT, dL, K, eL, Gy, aW, ff, hl, r, dq, en, g, t, gk, kP, iT, QT, at, m, bt); #c1(FANCA) c2(NP_001273096) c3(Fanconi anemia group A protein isoform c) c4(104320) c5( )c6( )c7(T, os, aC, QO, if, al, oY, oZ, qN, q, qK, aT, dL, K, eL, Gy, aW, ff, hl, r, dq, en, g, t, gk, kP, iT, QT, at, m, bt); #c1(FANCA) c2(XP_011521247) c3 (Fanconi anemia group A protein isoform X2) c4( )c5(316162) c6( ) c7(T, os, aC, QO, if, al, oY, oZ, qN, q, qK, aT, dL, K, eL, Gy, aW, ff, hl, r, dq, en, g, t, gk, kP, iT, QT, at, m, bt); #c1(FANCA) c2(XP_011521249) c3(Fanconi anemia group A protein isoform X3) c4( )c5(316163, 316164, 316165, 316166, 316167) c6( )c7(T, os, aC, QO, if, al, oY, oZ, qN, q, qK, aT, dL, K, eL, Gy, aW, ff, hl, r, dq, en, g, t, gk, kP, iT, QT, at, m, bt); #c1(FANCA) c2(XP_011521250) c3(Fanconi anemia group A protein isoform X4) c4(316168, 316169, 316170) c5( )c6( )c7(T, os, aC, QO, if, al, oY, oZ, qN, q, qK, aT, dL, K, eL, Gy, aW, ff, hl, r, dq, en, g, t, gk, kP, iT, QT, at, m, bt); #c1(FANCB) c2(NP_001018123) c3(Fanconi anemia group B protein) c4(27410, 27411) c5(27412, 27413, 27414, 27415, 27416, 27417, 27418, 27419, 27420, 27421, 27422, 27423, 27424, 27425, 27426, 27427) c6( )c7(T, os, ow, WL, qx, DA, bs, jh, oZ, hF, br, er, x, pp, oW, aT, dz, hn, iY, ox, jj, pq, Nr, iU, iZ, aW, po, brb, brc, ho, f, bw, Nq, ck, iT, kz, brd, cj, k, bt, kS); #c1(FANCC) c2(NP_001230672) c3(Fanconi anemia group C protein isoform a) c4(86535) c5(86536, 86537, 86538, 86539, 86540, 86541, 86542, 86543, 86544, 86545) c6( )c7(T, os, aC, bre, if, ew, oZ, qN, hF, azn, x, jz, aE, aT, h, dL, K, adV, Gc, aW, g, iK, aj, ck, iT, oX, m, bt, B, sH); #c1(FANCC) c2(XP_006717067) c3(Fanconi anemia group C protein isoform X6) c4(290678) c5(290679, 290680, 290681, 290682, 290683, 290684) c6( )c7(T, os, aC, bre, if, ew, oZ, qN, hF, azn, x, jz, aE, aT, h, dL, K, adV, Gc, aW, g, ik, aj, ck, iT, oX, m, bt, B, sH); #c1(FANCD2) c2(NP_001018125) c3(Fanconi anemia group D2 protein isoform b) c4(27428) c5(27429, 27430, 27431, 27432, 27433, 27434, 27435, 27436, 27437, 27438, 27439, 27440, 27441, 27442, 27443, 27444) c6( )c7(bL, T, os, aC, aaH, ew, fd, n, hy, oZ, q, ad, eA, ib, aE, aT, ta, cy, dL, K, aDZ, ak, qw, B, BN, wr, iU, aW, CW, r, dq, en, g, t, avf, KM, bw, dr, iP, avz, ck, iT, aWZ, hk, ajn, avF, wR, m, aan, bC, brf, bt, ne); #c1(FANCD2) c2(NP_149075) c3(Fanconi anemia group D2 protein isoform a) c4(227020) c5(227021, 227022, 227023, 227024, 227025, 227026) c6( )c7(bL, T, os, aC, aaH, ew, fd, n, hy, oZ, q, ad, eA, ib, aE, aT, ta, cy, dL, K, aDZ, ak, qw, B, BN, wr, il, aW, CW, r, dq, en, g, t, avf, KM, bw, dr, iP, avz, ck, iT, aWZ, hk, ajn, avF, wR, m, aan, bC, brf, bt, ne); #c1(FANCD2) c2(XP_011531781) c3(Fanconi anemia group D2 protein isoform X5) c4(327872) c5( )c6( )c7(bL, T, os, aC, aaH, ew, fd, n, hy, oZ, q, ad, eA, ib, aE, aT, ta, cy, dL, K, aDZ, ak, qw, B, BN, wr, il, aW, CW, r, dq, en, g, t, avf, KM, bw, dr, iP, avz, ck, iT, aWZ, hk, ajn, avF, wR, m, aan, bC, brf, bt, ne); #c1(FANCE) c2(NP_068741) c3(Fanconi anemia group E protein) c4(210130) c5(210131, 210132, 210133, 210134, 210135, 210136) c6( )c7(aC, os, aA, aT, oZ, v, bqS, m, g, aF); #c1(FANCF) c2(NP_073562) c3(Fanconi anemia group F protein) c4(212853) c5(212854, 212855, 212856, 212857, 212858, 212859) c6( )c7(ck, T, os, aC, cc, iT, aW, if, YZ, mx, ik, aT, oZ, m, ci, qN, dw, H, bt, g, t); #c1(FANCG) c2(NP_004620) c3(Fanconi anemia group G protein) c4(148249, 148250, 148251) c5(148252, 148253, 148254, 148255, 148256, 148257, 148258, 148259, 148260, 148261, 148262, 148263, 148264, 148265, 148266, 148267, 148268, 148269) c6( )c7(GI, T, os, aC, ht, hy, oZ, ad, aE, aT, Gw, dL, avn, brg, r, g, eM, iT, bdP, m, Gu, B); #c1(FANCI) c2(NP_001106849) c3(Fanconi anemia group 1 protein isoform 1) c4(42616, 42617) c5(42618, 42619, 42620, 42621, 42622, 42623) c6( )c7(brh, os, aT, oZ, ey, K, bX); #c1(FANCI) c2(NP_060663) c3(Fanconi anemia group 1 protein isoform 2) c4(197052, 197053) c5(197054, 197055, 197056, 197057, 197058, 197059, 197060, 197061, 197062, 197063, 197064, 197065) c6( )c7(brh, os, aT, oZ, ey, K, bX); #c1(FANCI) c2(XP_011520071) c3(Fanconi anemia group 1 protein isoform X6) c4( )c5(314964, 314965, 314966, 314967, 314968, 314969) c6( )c7(brh, os, aT, oZ, ey, K, bX); #c1(FANCL) c2(NP_001108108) c3(E3 ubiquitin-protein ligase FANCL isoform 1) c4(43133) c5(43134, 43135, 43136, 43137, 43138, 43139) c6( )c7(ck, av, aC, os, x, if, asl, aT, oZ, dL, bk, qN, g, hF, bt, eL, er); #c1(FANCL) c2(NP_060532) c3(E3 ubiquitin-protein ligase FANCL isoform 2) c4(196428) c5(196429, 196430, 196431, 196432, 196433, 196434, 196435, 196436, 196437, 196438, 196439, 196440) c6( )c7(ck, av, aC, os, x, if, asQ, aT, oZ, dl, bK, qN, g, hF, bt, eL, er); #c1 (FANCL) c2(XP_011531241) c3(E3 ubiquitin-protein ligase FANCL isoform X1) c4(327230) c5( )c6( )c7(ck, av, aC, os, x, if, asQ, aT, oZ, dL, bK, qN, g, hF, bt, eL, er); #c1(FANCM) c2(NP_065988) c3(Fanconi anemia group M protein) c4(206605) c5(206606, 206607, 206608, 206609, 206610, 206611) c6( )c7(T, os, aC, eH, awd, oW, r, aT, oZ, K, g, eF); #c1(FANCM) c2(XP_011535336) c3(Fanconi anemia group M protein isoform X1) c4( )c5(332335) c6( )c7(T, os, aC, eH, awd, oW, r, aT, oZ, K, g, eF); #c1(FANCM) c2(XP_011535338) c3(Fanconi anemia group M protein isoform X4) c4(332336) c5( )c6( )c7(T, os, aC, eH, awd, oW, r, aT, oZ, K, g, eF); #c1(FANKI) c2(NP_660278) c3(fibronectin type 3 and ankyrin repeat domains protein 1) c4(240618, 240619) c5(240620, 240621, 240622, 240623, 240624, 240625, 240626) c6( ) c7(bM, j, bK, ml); #c1(FANKI) c2(XP_006718121) c3(fibronectin type 3 and ankyrin repeat domains protein 1 isoform X5) c4( )c5(291523, 291524, 291525) c6( )c7(bM, j, bK, mQ); #c1(FANKI) c2(XP_006718127) c3(fibronectin type 3 and ankyrin repeat domains protein 1 isoform X8) c4(291526, 291527, 291528) c5(291529, 291530, 291531, 291532, 291533, 291534, 291535, 291536, 291537, 291538, 291539, 291540, 291541) c6( )c7(bM, j, bK, mQ); #c1 (FANKI) c2(XP_006718130) c3(fibronectin type 3 and ankyrin repeat domains protein 1 isoform X10) c4(291542) c5(291543, 291544, 291545, 291546, 291547) c6( )c7(bM, j, bK, ml); #c1(FANKI) c2(XP_011538652) c3(fibronectin type 3 and ankyrin repeat domains protein 1 isoform X3) c4(336391) c5(336392, 336393, 336394, 336395, 336396, 336397) c6( )c7(bM, j, bK, mQ); #c1(FANKI) c2(XP_011538659) c3(fibronectin type 3 and ankyrin repeat domains protein 1 isoform X9) c4(336398) c5( )c6( )c7(bM, j, bK, mQ); #c1(FANKI) c2(XP_011538660) c3(fibronectin type 3 and ankyrin repeat domains protein 1 isoform X11) c4( )c5(336399, 336400, 336401, 336402, 336403, 336404, 336405, 336406, 336407, 336408, 336409, 336410) c6( ) c7(bM, j, bK, mQ); #c1(FANKI) c2(XP_011538665) c3(fibronectin type 3 and ankyrin repeat domains protein 1 isoform X15) c4( ) c5(336411, 336412, 336413, 336414, 336415, 336416) c6( )c7(bM, j, bK, mQ); #c1(FAP) c2(NP_001278736) c3(prolyl endopeptidase FAP isoform 2) c4(110005) c5(110006, 110007, 110008, 110009, 110010, 110011) c6( )c7(T, AS, dA, fR, ob, aku, akw, asB, ht, al, aA, bB, n, jx, NT, aks, ci, fu, Jt, q, ad, ip, alZ, aHF, hd, aT, AN, ey, H, ae, mJ, BO, s, aW, Y, YC, r, ks, Kf, g, lm, t, bF, Yl, gk, bP, eM, nB, akp, bE, afd, cj, m, R, B); #c1(FAP) c2(NP_004451) c3(prolyl endopeptidase FAP isoform 1) c4(146425) c5(146426, 146427, 146428, 146429, 146430, 146431) c6( ) c7(T, AS, dA, fR, ob, aku, akw, asB, ht, al, aA, bB, n, jx, NT, aks, ci, fu, Jt, q, ad, ip, alZ, aHF, hd, aT, AN, ey, H, ae, mJ, BO, s, aW, Y, YC, r, ks, Kf, g, lm, t, bF, Yl, gk, bP, eM, nB, akp, bE, afd, cj, m, R, B); #c1(FAR1) c2(NP_115604) c3(fatty acyl-CoA reductase 1) c4(223347, 223348) c5(223349, 223350, 223351, 223352, 223353, 223354, 223355, 223356, 223357, 223358, 223359, 223360) c6( )c7(WI); #c1(FAR1) c2(XP_011518703) c3(fatty acyl-CoA reductase 1 isoform X2) c4( )c5(313078) c6( )c7(WI); #c1(FARP1) c2(NP_001001715) c3(FERM, RhoGEF and pleckstrin domain-containing protein 1 isoform 2) c4(18989) c5(18990, 18991, 18992, 18993, 18994, 18995) c6( )c7(hb, lB, af, Ea, an); #c1(FARP1) c2(NP_001273768) c3(FERM, RhoGEF and pleckstrin domain-containing protein 1 isoform 3) c4(105828, 105829) c5(105830, 105831, 105832, 105833, 105834, 105835, 105836, 105837, 105838, 105839, 105840, 105841, 105842) c6( )c7(hb, lB, af, Ea, an); #c1(FARP1) c2(NP_005757) c3(FERM, RhoGEF and pleckstrin domain-containing protein 1 isoform 1) c4(159108, 159109, 159110) c5(159111, 159112, 159113, 159114, 159115, 159116, 159117, 159118, 159119, 159120, 159121, 159122, 159123, 159124, 159125, 159126, 159127) c6( ) c7(hb, lB, af, Ea, an); #c1(FARP2) c2(NP_001269912) c3(FERM, RhoGEF and pleckstrin domain-containing protein 2 isoform b) c4(103155) c5(103156, 103157, 103158, 103159, 103160, 103161) c6( )c7(Cv, ck, bri, cj, aYK, bN); #c1(FARP2) c2(NP_001269913) c3(FERM, RhoGEF and pleckstrin domain-containing protein 2 isoform c) c4(103162, 103163) c5(103164, 103165, 103166, 103167, 103168, 103169, 103170, 103171, 103172, 103173, 103174, 103175, 103176, 103177, 103178, 103179, 103180, 103181, 103182) c6( )c7(Cv, ck, bri, cj, aYK, bN); #c1(FARP2) c2(NP_055623) c3(FERM, RhoGEF and pleckstrin domain-containing protein 2 isoform a) c4(182322, 182323) c5(182324, 182325, 182326, 182327, 182328, 182329) c6( )c7(Cv, ck, bri, cj, aYK, bN); #c1(FARP2) c2(XP_011510539) c3(FERM, RhoGEF and pleckstrin domain-containing protein 2 isoform X3) c4(301067) c5(301068, 301069, 301070, 301071, 301072, 301073) c6( )c7(Cv, ck, bri, cj, aYK, bN); #c1(FARS2) c2(XP_005248868) c3(phenylalanine-tRNA ligase, mitochondrial isoform X1) c4(268699) c5(268700, 268701, 268702, 268703, 268704, 268705, 268706, 268707, 268708, 268709) c6( )c7(Fr, hb, brj, ha, dF, cd, Ft); #c1(FARS2) c2(XP_006715029) c3(phenylalanine-tRNA ligase, mitochondrial isoform X7) c4(288521, 288522) c5(288523, 288524, 288525, 288526, 288527, 288528, 288529, 288530, 288531, 288532, 288533, 288534) c6( )c7(Fr, hb, brj, ha, dF, cd, Ft); #c1(FARSA) c2(NP_004452) c3(phenylalanine-tRNA ligase alpha subunit) c4(146432, 146433) c5(146434, 146435, 146436, 146437, 146438, 146439, 146440, 146441, 146442, 146443, 146444, 146445) c6( )c7(iT, wT); #c1(FAS) c2(NP_000034) c3(tumor necrosis factor receptor superfamily member 6 isoform 1 precursor) c4(316, 317, 318) c5(319, 320, 321, 322, 323, 324, 325, 326, 327, 328, 329, 330) c6( ) c7(bM, T, aC, fR, pf, if, al, eb, brl, bB, cl, fd, qj, ci, bh, amW, aR, brq, x, afr, xy, aE, Bc, fm, bW, dV, ja, K, hu, dQ, hH, DY, dF, bc, aiJ, el, Qe, kS, JI, aW, az, hl, wf, fc, hN, vs, brr, apm, brk, g, as, uC, eF, GR, cm, mK, bw, y, fT, eM, nB, iT, aDs, vA, mx, bru, cj, PV, uo, dW, k, m, eg, cE, iu, gU, sQ, wu, sw, aw, ht, fN, n, BH, aj, aGU, oY, qN, fr, qW, cd, eW, brn, alu, jz, cc, kH, Lv, lG, CU, By, dL, DU, h, hB, eV, A, CM, tx, fQ, aq, ks, aVD, pm, dw, Wt, iO, GG, dB, hk, eD, brt, hL, at, oh, ut, SZ, fl, brs, jd, Pr, B, it, bL, Ob, Dw, EM, FM, AL, fG, hx, yk, jx, ex, aA, cz, fF, hy, v, OY, GC, C, aYW, V, q, J, o, eA, bjA, fh, nX, afQ, yg, aT, vH, cy, bq, an, ae, af, eu, Su, jf, hM, s, ND, vT, iU, Y, Lf, xR, r, lo, en, hf, bu, t, kC, ik, brm, pb, vk, Cv, ck, sS, OW, Dy, c, ac, pZ, nM, hc, aGo, iM, RW, Da, dy, aEr, bt, ao, Kd, id, Ke, MH, iW, jh, lD, bS, OH, fu, mC, ad, KL, hX, dP, av, hd, F, io, hD, ji, lQ, jo, wm, yF, ul, il, d, BN, lU, je, ff, eH, eZ, brp, aF, hp, iV, iR, bz, Hv, LB, D, gk, Bv, eU, bE, pE, Ny, do, X, bro, Lb, cP, od, adg, hE, xg, ne, hv, pC); #c1(FAS) c2(NP_690610) c3(tumor necrosis factor receptor superfamily member 6 isoform 2 precursor) c4(244917) c5(244918, 244919, 244920, 244921, 244922, 244923) c6( )c7(bM, T, aC, fR, pf, if, al, eb, brl, bB, cL, fd, qj, ci, bh, amW, aR, brq, x, afr, xy, aE, Bc, fm, bW, dV, ja, K, hu, dQ, hH, DY, dF, bc, aiJ, eL, De, KS, JI, aW, az, hl, wf, fc, hN, vs, brr, apm, brk, g, as, uC, eF, GR, cm, mK, bw, y, fT, eM, nB, iT, aDs, vA, mx, bru, cj, PV, uo, dW, k, m, eg, cE, iu, gU, sl, wu, sw, aw, ht, fN, n, BH, aj, aGU, oY, qN, fr, qW, cd, eW, brn, aDu, jz, cc, KH, Lv, lG, cU, By, dL, DU, h, hB, eV, A, CM, tx, fQ, aq, ks, aVD, pm, dw, Wt, iO, GG, dB, hk, eD, brt, hL, at, oh, ut, SZ, FL, brs, jd, Pr, B, it, bL, Ob, Dw, EM, fM, AL, fG, hx, yk, jx, eX, aA, cz, fF, hy, v, OY, GC, C, aYW, V, q, J, o, eA, bjA, fh, nX, afQ, yg, aT, vH, cy, bq, an, ae, af, eu, Su, jf, HM, s, ND, vT, iU, Y, Lf, xR, r, lo, en, hf, bu, t, kC, iK, brm, pb, vk, Cv, ck, sS, OW, Dy, c, ac, pZ, nM, hc, aGo, iM, RW, Da, dy, aEr, bt, ao, Kd, id, Ke, MH, iW, jh, lD, bS, OH, fu, mC, ad, KL, hX, dP, av, hd, F, io, hD, ji, lQ, jo, wm, yF, ul, il, d, BN, lU, je, ff, eH, eZ, brp, aF, hp, iV, iR, bz, Hv, LB, D, gk, Bv, eU, bE, pE, Ny, do, X, bro, Lb, cP, oQ, adg, hE, xg, ne, hv, pC); #c1(FAS) c2(NP_69061 l) c3(tumor necrosis factor receptor superfamily member 6 isoform 3 precursor) c4( )c5(244924, 244925, 244926, 244927, 244928, 244929) c6( )c7(bM, T, aC, fR, pf, if, al, eb, brl, bB, cL, fd, qj, ci, bh, amW, aR, brq, x, afr, xy, aE, Bc, fm, bW, dV, ja, K, hu, dQ, hH, DY, dF, bc, aiJ, eL, Qe, kS, JI, aW, az, hl, wf, fc, hN, vs, brr, apm, brk, g, as, uC, eF, GR, cm, mK, bw, y, fT, eM, nB, iT, aDs, vA, mx, bru, cj, PV, uo, dW, k, m, eg, cE, iu, gU, sl, wu, sw, aw, ht, lN, n, BH, aj, aGU, oY, qN, fr, qW, cd, eW, brn, alu, jz, cc, kH, Lv, lG, cU, By, dL, DU, h, hB, eV, A, CM, tx, fQ, aq, ks, aVD, pm, dw, Wt, iO, GG, dB, hk, eD, brt, hL, at, oh, ut, SZ, fL, brs, jd, Pr, B, it, bL, Ob, Dw, EM, fM, AL, fG, hx, yk, jx, eX, aA, cz, fF, hy, v, OY, GC, C, aYW, V, q, J, o, eA, bjA, fh, nX, afQ, yg, aT, vH, cy, bq, an, ae, af, eu, Su, jf, hM, s, ND, vT, iU, Y, Lf, xR, r, lo, en, hf, bu, t, kC, iK, brm, pb, vk, Cv, ck, sS, OW, Dy, c, ac, pZ, nM, hc, aGo, iM, RW, Da, dy, aEr, bt, ao, Kd, id, Ke, MH, iW, jh, lD, bS, OH, fu, mC, ad, kL, hX, dP, av, hd, F, io, hD, ji, lQ, jo, wm, yF, ul, il, d, BN, lU, je, ff, eH, eZ, brp, aF, hp, iV, iR, bz, Hv, LB, D, gk, Bv, eU, bE, pE, Ny, do, X, bro, Lb, cP, oQ, adg, hE, xg, ne, hv, pC); #c1(FAS) c2(XP_011538068) c3(tumor necrosis factor receptor superfamily member 6 isoform X3) c4(335609) c5(335610, 335611, 335612, 335613, 335614, 335615) c6( )c7(bM, T, aC, fR, pf, if, al, eb, brl, bB, cL, fd, qj, ci, bh, amW, aR, brq, x, afr, xy, aE, Bc, fm, bW, dV, ja, K, hu, dQ, hH, DY, dF, bc, aiJ, eL, Qe, KS, JI, aW, az, hl, wf, fc, hN, vs, brr, apm, brk, g, as, uC, eF, GR, cm, mk, bw, y, fT, eM, nB, iT, aDs, vA, mx, bru, cj, PV, uo, dW, k, m, eg, cE, iu, gU, sQ, wu, sw, aw, ht, fN, n, BH, aj, aGU, oY, qN, fr, qW, cd, eW, brn, alu, jz, cc, kH, Lv, lG, cU, By, dL, DU, h, hB, eV, A, CM, tx, fQ, aq, ks, aVD, pm, dw, Wt, iO, GG, dB, hk, eD, brt, hL, at, oh, ut, SZ, fl, brs, jd, Pr, B, it, bL, Ob, Dw, EM, fM, AL, fG, hx, yk, jx, eX, aA, cz, fF, hy, v, OY, GC, C, aYW, V, q, J, o, eA, bjA, fh, nX, afQ, yg, aT, vH, cy, bq, an, ae, af, eu, Su, jf, hM, s, ND, vT, iU, Y, Lf, xR, r, lo, en, hf, bu, t, kC, ik, brm, pb, vk, Cv, ck, sS, OW, Dy, c, ac, pZ, nM, hc, aGo, iM, RW, Da, dy, aEr, bt, ao, Kd, id, Ke, MH, iW, jh, lD, bS, oH, fu, mC, ad, kL, hX, dP, av, hd, F, io, hD, ji, lQ, jo, wm, yF, ul, iQ, d, BN, lU, je, ff, eH, eZ, brp, aF, hp, iV, iR, bz, Hv, LB, D, gk, Bv, eU, bE, pE, Ny, dO, X, bro, Lb, cP, oQ, adg, hE, xg, ne, hv, pC); #c1(FASLG) c2(NP_000630) c3(tumor necrosis factor ligand superfamily member 6 isoform 1) c4(14105) c5(14106, 14107, 14108, 14109, 14110, 14/11, 14112, 14113, 14114) c6(14115, 14116) c7(bM, T, aC, xS, fR, pf, if, fd, brl, bB, ur, ci, qj, Ul, amW, aR, xy, dY, aE, fm, fS, hu, brw, eL, bc, aiJ, hm, De, JI, aW, LZ, g, cm, uC, mK, fT, eM, nB, iT, aDs, mx, cj, PV, uo, dW, m, cE, dD, gU, sQ, wu, sw, ht, fN, n, BH, aj, fr, qN, brv, cc, h, dL, DU, ey, hB, A, ks, aq, Og, aVD, fq, iO, dB, hk, Fi, PI, at, ut, fL, lB, Pr, B, bL, Ob, fM, AL, jx, aA, fF, hz, hy, C, aYW, aoC, V, q, er, eA, afd, aT, vH, cy, jj, bq, an, ae, af, eu, jf, s, ND, iU, Y, fO, r, en, cl, t, aER, ik, Cv, ck, sS, OW, Dy, pZ, ac, il, RW, Da, UF, bt, Kd, id, jh, vr, bS, oH, jE, eq, mC, ad, hX, dP, av, hd, F, oW, io, hD, lQ, wm, DI, d, BN, lU, ff, Of, cr, eZ, brp, aF, hp, bz, D, bE, Ny, hE, kS, X, y, pC); #c1(FASLG) c2(NP_001289675) c3(tumor necrosis factor ligand superfamily member 6 isoform 2) c4(114352) c5(114353, 114354, 114355, 114356, 114357, 114358) c6(114359) c7(bM, T, aC, xS, fR, pf, if, fd, brl, bB, ur, ci, qj, Ul, amW, aR, xy, dY, aE, fm, fS, hu, brw, eL, bc, aiJ, hm, Qe, JI, aW, LZ, g, cm, uC, mk, fT, eM, nB, iT, aDs, mx, cj, PV, uo, dW, m, cE, dD, gU, sl, wu, sw, ht, fN, n, BH, aj, fr, qN, brv, cc, h, dL, DU, ey, hB, A, ks, aq, Og, aVD, fq, iO, dB, hk, Fi, PI, at, ut, fL, lB, Pr, B, bL, Ob, fM, AL, jx, aA, fF, hz, hy, C, aYW, aoC, V, q, er, eA, afQ, aT, vH, cy, jj, bq, an, ae, af, eu, jf, s, ND, iU, Y, fO, r, en, cl, t, aER, ik, Cv, ck, sS, OW, Dy, pZ, ac, il, RW, Da, UF, bt, Kd, id, jh, vr, bS, OH, jE, eq, mC, ad, hX, dP, av, hd, F, oW, io, hD, lQ, wm, DI, d, BN, lU, ff, Of, cr, eZ, brp, aF, hp, bz, D, bE, Ny, hE, KS, X, y, pC); #c1(FASN) c2(NP_004095) c3(fatty acid synthase) c4(143513, 143514) c5(143515, 143516, 143517, 143518, 143519, 143520, 143521, 143522, 143523, 143524, 143525, 143526) c6( )c7(bM, T, fR, pf, if, al, fd, brl, bB, cL, gf, qj, ci, bh, ip, x, xy, bW, dV, K, hu, DY, dF, bc, eL, JI, aW, az, hl, fc, hN, apm, g, as, eF, cm, bw, mK, eM, nB, vA, bru, PV, dW, k, m, in, cE, iu, aw, ht, n, aj, aGU, fr, nA, qN, eW, brn, jz, kH, h, jy, eV, dL, hB, cY, A, tx, fe, aq, kp, iO, mg, hL, oh, SZ, fL, qF, Pr, B, it, bL, fM, eR, AL, jx, eX, aA, GC, v, V, q, J, o, Ql, eA, bjA, fh, aT, vH, cy, ae, af, s, ND, vT, oV, Y, Lf, xR, r, lo, en, bu, cl, t, ik, brm, pb, ck, sS, OW, Dy, ac, nM, iM, hc, au, wR, bt, ao, id, iW, jh, Fb, mC, ad, hX, av, hd, F, aVq, io, hD, ji, fb, yF, d, je, CH, ff, eH, eZ, aF, hp, iV, bz, LB, gk, eU, pu, bE, do, bro, cP, pK, y, xg, X, pC); #c1(FASTK) c2(NP_001245390) c3(fas-activated serine/threonine kinase isoform 5) c4(93436) c5(93437, 93438, 93439, 93440, 93441, 93442) c6( )c7(qe, eM, as, ER, Bh, aMk); #c1(FASTK) c2(NP_006703) c3(fas-activated serine/threonine kinase isoform 1) c4(167254) c5(167255, 167256, 167257, 167258, 167259, 167260) c6( )c7(qe, eM, as, ER, Bh, aMk); #c1(FASTK) c2(NP_148936) c3(fas-activated serine/threonine kinase isoform 4) c4(226629) c5(226630, 226631, 226632, 226633, 226634, 226635) c6( )c7(ge, eM, as, ER, Bh, aMk); #c1(FASTK) c2(XP_011514063) c3(fas-activated serine/threonine kinase isoform X2) c4(306605, 306606) c5(306607, 306608, 306609, 306610, 306611, 306612) c6( )c7(qe, eM, as, ER, Bh, aMk); #c1(FASTK) c2(XP_011514064) c3(fas-activated serine/threonine kinase isoform X3) c4( )c5(306613, 306614, 306615, 306616, 306617, 306618) c6( )c7(qe, eM, as, ER, Bh, aMk); #c1(FASTKD2) c2(NP_055744) c3(FAST kinase domain-containing protein 2) c4(183229, 183230) c5(183231, 183232, 183233, 183234, 183235, 183236, 183237, 183238, 183239, 183240, 183241, 183242, 183243, 183244, 183245, 183246, 183247, 183248, 183249, 183250) c6( )c7(T, aW, aT, ey, acJ, px); #c1(FAT1) c2(XP_006714202) c3(protocadherin Fat 1 isoform X1) c4(287637, 287638, 287639) c5(287640, 287641, 287642, 287643, 287644, 287645, 287646, 287647, 287648, 287649, 287650, 287651, 287652, 287653, 287654, 287655, 287656, 287657, 287658) c6( )c7(bL, T, e, EQ, aj, hy, v, q, J, ad, eA, x, aE, qK, aT, bK, ae, af, kl, d, B, aW, Y, oV, fc, ff, r, en, bz, ck, Fi, cj, k, cP, ei, bt, it, X); #c1(FAT2) c2(XP_011535905) c3(protocadherin Fat 2 isoform X1) c4(333163) c5(333164, 333165, 333166, 333167, 333168, 333169) c6( )c7(it, iu); #c1(FAT3) c2(NP_001008781) c3(protocadherin Fat 3 precursor) c4(23403, 23404) c5(23405, 23406, 23407, 23408, 23409, 23410, 23411, 23412, 23413, 23414, 23415) c6( )c7(it, ip); #c1(FAT3) c2(XP_011540886) c3(protocadherin Fat 3 isoform X1) c4(339426) c5(339427, 339428, 339429, 339430, 339431, 339432, 339433) c6( )c7(it, ip); #c1(FAT4) c2(NP_078858) c3(protocadherin Fat 4 isoform 3 precursor) c4(215812, 215813) c5(215814, 215815, 215816, 215817, 215818, 215819, 215820, 215821, 215822, 215823, 215824, 215825) c6( )c7(T, bdG, ac, aW, Y, oe, ht, aT, aA, it, afw, j, bK, bz, hu, g, brx, fq, B, Hr); #c1(FATE1) c2(NP_149076) c3(fetal and adult testis-expressed transcript protein) c4(227027) c5(227028, 227029, 227030, 227031, 227032, 227033) c6( )c7(T, gU, aW, aT, nY, bu, V, ra, B, ek); #c1(FAU) c2(NP_001988) c3 (ubiquitin-like protein fubi and ribosomal protein S30 precursor) c4(122880, 122881, 122882, 122883, 122884, 122885, 122886, 122887, 122888, 122889, 122890) c5(122891, 122892, 122893, 122894, 122895, 122896, 122897, 122898, 122899, 122900, 122901, 122902, 122903, 122904, 122905, 122906, 122907, 122908, 122909, 122910, 122911, 122912, 122913, 122914, 122915, 122916, 122917, 122918, 122919, 122920, 122921, 122922, 122923, 122924, 122925, 122926, 122927, 122928, 122929, 122930, 122931, 122932) c6( )c7(eF); #c1(FBF1) c2(NP_001074011) c3(fas-binding factor 1) c4(36944, 36945, 36946, 36947) c5(36948, 36949, 36950, 36951, 36952, 36953, 36954, 36955, 36956, 36957, 36958, 36959, 36960, 36961, 36962, 36963, 36964, 36965, 36966, 36967, 36968, 36969, 36970, 36971, 36972) c6( )c7(Rs, B, jE); #c1(FBL) c2(NP_001427) c3(rRNA 2′-O-methyltransferase fibrillarin) c4(117775) c5( ) c6( )c7(T, qt, aW, hl, aT, h, aq, lR, K, Dz, pb, B, ad); #c1(FBL) c2(XP_011524925) c3(rRNA 2′-O-methyltransferase fibrillarin isoform X2) c4( )c5(319406, 319407, 319408, 319409, 319410, 319411) c6( )c7(T, qt, aW, hl, aT, h, aq, lR, K, Dz, pb, B, ad); #c1(FBLIM1) c2(XP_011539920) c3(filamin-binding LIM protein 1 isoform X4) c4(338215) c5(338216, 338217, 338218, 338219, 338220, 338221, 338222, 338223, 338224, 338225, 338226, 338227) c6( )c7(T, ha, fd, aon, eA, b, aFl, hW, aeO, aT, K, Tj, aeR, aW, eH, ark, eF, Ew, aGY, cj, ER, i, lf); #c1(FBLIM1) c2(XP_011539921) c3(filamin-binding LIM protein 1 isoform X3) c4(338228, 338229, 338230, 338231, 338232, 338233) c5(338234, 338235, 338236, 338237, 338238, 338239, 338240, 338241, 338242, 338243, 338244, 338245, 338246, 338247, 338248, 338249, 338250, 338251, 338252, 338253, 338254, 338255, 338256, 338257, 338258, 338259, 338260, 338261, 338262, 338263, 338264, 338265, 338266, 338267, 338268, 338269, 338270, 338271, 338272, 338273, 338274, 338275) c6( ) c7(T, ha, fd, aon, eA, b, aFl, hW, aeO, aT, K, Tj, aeR, aW, eH, ark, eF, Ew, aGY, cj, ER, i, lf); #c1(FBLN1) c2(NP_001987) c3(fibulin-1 isoform C precursor) c4(122858, 122859, 122860, 122861, 122862, 122863) c5(122864, 122865, 122866, 122867, 122868, 122869, 122870, 122871, 122872, 122873, 122874, 122875, 122876, 122877, 122878, 122879) c6( )c7(bM, T, lP, e, aA, aHC, bry, ci, zU, fr, ad, ip, dP, Bc, bpp, aT, bK, brA, MO, ae, eG, aW, Y, PX, ev, af, brz, t, atb, op, sn, Dy, m, bD, B); #c1(FBLN1) c2(NP_006476) c3(fibulin-1 isoform B precursor) c4(165489) c5(165490, 165491, 165492, 165493, 165494, 165495, 165496, 165497, 165498, 165499, 165500, 165501, 165502, 165503, 165504, 165505) c6( )c7(bM, T, lP, e, aA, aHC, bry, ci, zU, fr, ad, ip, dP, Bc, bpp, aT, bK, brA, MO, ae, eG, aW, Y, PX, ev, aF, brz, t, atb, op, sn, Dy, m, bD, B); #c1(FBLN1) c2(NP_006477) c3(fibulin-1 isoform D precursor) c4(165506, 165507, 165508, 165509, 165510) c5(165511, 165512, 165513, 165514, 165515, 165516, 165517, 165518, 165519, 165520, 165521, 165522, 165523, 165524, 165525, 165526, 165527, 165528, 165529, 165530, 165531, 165532, 165533) c6(165534) c7(bM, T, lP, e, aA, aHC, bry, ci, zU, fr, ad, ip, dP, Bc, bpp, aT, bK, brA, MO, ae, eG, aW, Y, PX, ev, aF, brz, t, atb, op, sn, Dy, m, bD, B); #c1(FBLN1) c2(NP_006478) c3(fibulin-1 isoform A precursor) c4( )c5(165535, 165536, 165537, 165538, 165539, 165540, 165541, 165542, 165543, 165544, 165545, 165546, 165547, 165548, 165549, 165550, 165551, 165552, 165553, 165554, 165555, 165556, 165557, 165558, 165559, 165560, 165561, 165562, 165563, 165564, 165565, 165566, 165567, 165568, 165569, 165570, 165571, 165572, 165573, 165574) c6(165575) c7(bM, T, lP, e, aA, aHC, bry, ci, zU, fr, ad, ip, dP, Bc, bpp, aT, bK, brA, MO, ae, eG, aW, Y, PX, ev, aF, brz, t, atb, op, sn, Dy, m, bD, B); #c1(FBLN2) c2(NP_001158507) c3(fibulin-2 isoform a precursor) c4(63679, 63680) c5(63681, 63682, 63683, 63684, 63685, 63686, 63687, 63688, 63689, 63690, 63691) c6( )c7(iu, T, aE, aW, brB, beA, aT, cz, brC, ci, g, af, vk); #c1(FBLN2) c2(NP_001989) c3 (fibulin-2 isoform b precursor) c4(122933, 122934) c5(122935, 122936, 122937, 122938, 122939, 122940, 122941, 122942, 122943, 122944, 122945, 122946, 122947, 122948, 122949, 122950, 122951, 122952, 122953, 122954) c6( ) c7(iu, T, aE, aW, brB, beA, aT, cz, brC, ci, g, af, vk); #c1(FBLN5) c2(NP_006320) c3(fibulin-5 precursor) c4(164006) c5(164007, 164008, 164009, 164010, 164011, 164012, 164013, 164014, 164015, 164016, 164017, 164018) c6( )c7(blK, T, dD, bM, id, lP, e, alv, blH, ci, fu, bX, eq, fr, brD, sD, cd, ad, av, afm, le, bkc, brC, dL, bU, brE, eL, blG, bz, W, Uj, bm, aS, blL, lC); #c1(FBLN5) c2(XP_011534659) c3(fibulin-5 isoform X3) c4(331166) c5( )c6( )c7(blK, T, dD, bM, id, lP, e, alv, blH, ci, fu, bX, eq, fr, brD, sD, cd, ad, av, afm, le, bkc, brC, dL, bU, brE, eL, blG, bz, W, Uj, bm, aS, blL, lC); #c1(FBN1) c2(NP_000129) c3(fibrillin-1 precursor) c4(2859, 2860, 2861) c5(2862, 2863, 2864, 2865, 2866, 2867, 2868, 2869, 2870, 2871, 2872, 2873, 2874, 2875, 2876, 2877, 2878, 2879) c6( )c7(T, Ap, rV, tQ, e, Av, lR, ci, brJ, bW, K, aW, eF, GR, zV, qJ, vA, mx, uo, m, vF, brN, Vj, aTe, brF, vl, brL, FG, wJ, n, Mi, brK, cd, no, azj, KN, jy, MX, mR, As, Vt, dw, fq, op, vD, Be, uz, vQ, bL, tf, eR, mr, DD, cz, lj, j, Eh, Mg, KB, brH, Er, aT, bK, bq, an, bf, brl, Ue, En, t, pb, asw, pZ, hc, uS, DE, Ep, Mt, ao, bgQ, jh, dG, sg, dP, hd, oj, ji, eH, brM, BG, dr, qa, LB, Bv, brG, aS, be, jr); #c1(FBN2) c2(NP_001990) c3(fibrillin-2 precursor) c4(122955) c5(122956, 122957, 122958, 122959, 122960, 122961, 122962, 122963, 122964, 122965, 122966) c6( )c7(bL, T, vl, eR, brl, e, aw, l, lj, bB, aj, v, j, ci, zU, lR, fr, aY, q, J, ft, no, aE, py, db, aT, h, jf, s, aW, r, hN, t, bz, pb, dr, LB, W, eM, bE, uo, m, pw, be, bt, us, brF); #c1(FBN3) c2(NP_115823) c3(fibrillin-3 precursor) c4(224134, 224/35) c5(224136, 224137, 224138, 224139, 224140, 224141, 224142, 224143, 224144, 224145, 224146, 224147, 224148, 224149, 224150, 224151, 224152, 224153, 224154, 224155, 224156, 224157) c6(224158, 224159, 224160, 224161, 224162, 224163) c7(Hd, kl, aZ, cz, m, bQ, t); #c1(FBP1) c2(NP_000498) c3(fructose-1,6-bisphosphatase 1) c4(10294, 10295) c5(10296, 10297, 10298, 10299, 10300, 10301, 10302, 10303, 10304, 10305, 10306) c6( )c7(Qi, brD, B, py, eM, bE, aW, aA, aT, bB, aq, aF, ci, an, iR, X); #c1(FBP1) c2(XP_006717068) c3(fructose-1,6-bisphosphatase 1 isoform X1) c4( )c5(290685, 290686, 290687, 290688, 290689) c6( )c7(Qi, brl, B, py, eM, bE, aW, aA, aT, bB, aq, aF, ci, an, iR, X); #c1(FBP2) c2(NP_003828) c3(fructose-1,6-bisphosphatase isozyme 2) c4(14/193) c5(141194, 141195, 141196, 141197, 141198, 141199) c6( )c7(Qi, aF, aA); #c1(FBRS) c2(NP_001098549) c3(probable fibrosin-1) c4(41374) c5( ) c6( )c7(bM, bP, vA, ff, aq, en, iR, cm, ae, B, op, ad); #c1(FBRS) c2(XP_011544218) c3(probable fibrosin-1 isoform X2) c4(343363) c5( ) c6( )c7(bM, bP, vA, ff, aq, en, iR, cm, ae, B, op, ad); #c1(FBRS) c2(XP_011544221) c3(probable fibrosin-1 isoform X5) c4( )c5(343364, 343365, 343366, 343367, 343368, 343369, 343370, 343371, 343372, 343373, 343374, 343375, 343376, 343377, 343378) c6( ) c7(bM, bP, vA, ff, aq, en, iR, cm, ae, B, op, ad); #c1(FBXL15) c2(NP_077302) c3(F-box/LRR-repeat protein 15) c4( )c5(215126, 215127, 215128, 215129, 215130, 215131, 215132, 215133, 215134, 215135, 215136, 215137, 215138, 215139, 215140, 215141, 215142, 215143, 215144, 215145, 215146, 215147, 215148, 215149, 215150) c6( )c7(rz, bO, Ko, iV, he, bN); #c1(FBXL15) c2(XP_005270206) c3(F-box/LRR-repeat protein 15 isoform X2) c4(283271, 283272, 283273, 283274, 283275, 283276) c5(283277, 283278, 283279, 283280, 283281, 283282, 283283, 283284, 283285, 283286, 283287) c6( )c7(rz, bO, Ko, iV, he, bN); #c1(FBXL17) c2(NP_001156787) c3(F-box/LRR-repeat protein (7) c4(61969, 61970) c5(61971, 61972, 61973, 61974, 61975, 61976, 61977, 61978, 61979, 61980, 61981, 61982) c6( )c7(aT); #c1(FBXL17) c2(XP_005272105) c3(F-box/LRR-repeat protein 17 isoform X1) c4(283700) c5(283701, 283702, 283703, 283704, 283705, 283706) c6( )c7(al); #c1(FBXL19) c2(NP_001093254) c3(F-box/LRR-repeat protein 19 isoform 1) c4(40188, 40189, 40190) c5(40191, 40192, 40193, 40194, 40195, 40196, 40197, 40198, 40199, 40200, 40201, 40202, 40203, 40204, 40205, 40206, 40207, 40208) c6( )c7(wD, ht, cr, hu); #c1(FBXL2) c2(NP_001165184) c3(F-box/LRR-repeat protein 2 isoform 2) c4(67528) c5(67529, 67530, 67531, 67532, 67533, 67534, 67535, 67536, 67537, 67538) c6( )c7(x, T, jh, k, hH, bt, af); #c1(FBXL2) c2(NP_036289) c3(F-box/LRR-repeat protein 2 isoform 1) c4(172861, 172862, 172863) c5(172864, 172865, 172866, 172867, 172868, 172869, 172870, 172871, 172872, 172873, 172874, 172875, 172876) c6( )c7(x, T, jh, k, hH, bt, af); #c1(FBXL2) c2(XP_005265071) c3(F-box/LRR-repeat protein 2 isoform X1) c4(279418) c5(279419) c6( )c7(x, T, jh, k, hH, bt, af); #c1(FBXL2) c2(XP_005265074) c3(F-box/LRR-repeat protein 2 isoform X3) c4( )c5(279420, 279421, 279422, 279423, 279424) c6( )c7(x, T, jh, k, hH, bt, af); #c1(FBXL2) c2(XP_011531861) c3(F-box/LRR-repeat protein 2 isoform X2) c4( ) c5(327924, 327925, 327926, 327927, 327928) c6( )c7(x, T, jh, k, hH, bt, af); #c1(FBXL20) c2(NP_001171835) c3(F-box/LRR-repeat protein 20 isoform 2) c4(71944) c5(71945, 71946, 71947, 71948, 71949, 71950) c6( )c7(bc); #c1(FBXL20) c2(NP_116264) c3(F-box/LRR-repeat protein 20 isoform 1) c4(226027) c5(226028, 226029, 226030, 226031, 226032, 226033) c6( )c7(bc); #c1(FBXL3) c2(NP_036290) c3(F-box/LRR-repeat protein 3) c4(172877) c5(172878, 172879, 172880, 172881, 172882, 172883) c6( )c7(jh); #c1(FBXL4) c2(NP_001265645) c3(F-box/LRR-repeat protein 4) c4(99087) c5(99088, 99089, 99090, 99091, 99092, 99093, 99094, 99095, 99096, 99097) c6( )c7(px, brP, ey); #c1(FBXL5) c2(NP_001180464) c3(F-box/LRR-repeat protein 5 isoform 4) c4(74140) c5(74141, 74142, 74143, 74144, 74145, 74146, 74147, 74148, 74149, 74150, 74151, 74152) c6( )c7(aF, aA); #c1(FBXL5) c2(NP_036293) c3(F-box/LRR-repeat protein 5 isoform 1) c4(172884, 172885) c5(172886, 172887, 172888, 172889, 172890, 172891) c6( )c7(aF, aA); #c1(FBXL7) c2(NP_036436) c3(F-box/LRR-repeat protein 7 isoform 1) c4(173842, 173843) c5(173844, 173845, 173846, 173847, 173848, 173849, 173850, 173851, 173852, 173853, 173854, 173855) c6( )c7(aW, aj, oe, cd, aT); #c1(FBX010) c2(XP_005251496) c3(F-box only protein 10 isoform X2) c4( )c5(270961, 270962, 270963, 270964, 270965, 270966) c6( )c7(aW, zP, K, aT); #c1(FBX010) c2(XP_006716817) c3(F-box only protein 10 isoform X3) c4(290503) c5(290504, 290505, 290506, 290507, 290508) c6( )c7(aW, zP, K, aT); #c1(FBX010) c2(XP_011516147) c3(F-box only protein 10 isoform X1) c4(309374, 309375) c5(309376, 309377, 309378, 309379, 309380, 309381, 309382) c6( )c7(aW, zP, K, aT); #c1(FBX011) c2(NP_001177203) c3(F-box only protein 11 isoform 4) c4(72515, 72516, 72517) c5(72518, 72519, 72520, 72521, 72522, 72523, 72524, 72525, 72526, 72527, 72528, 72529, 72530, 72531, 72532, 72533, 72534, 72535) c6( )c7(aBU, zo, fM, qj, pC); #c1(FBX011) c2(NP_079409) c3(F-box only protein 11 isoform 1) c4(217994) c5(217995, 217996, 217997, 217998, 217999, 218000, 218001, 218002, 218003, 218004) c6( )c7(aBU, zo, fM, qj, pC); #c1(FBX015) c2(NP_001136430) c3(F-box only protein 15 isoform 2) c4(54953, 54954) c5(54955, 54956, 54957, 54958, 54959, 54960, 54961) c6( )c7(W, fD); #c1(FBX015) c2(NP_689889) c3(F-box only protein 15 isoform 1) c4( )c5(244271, 244272, 244273, 244274, 244275) c6( )c7(W, fD); #c1(FBX017) c2(NP_079183) c3(F-box only protein 17 isoform 2) c4(217333) c5(217334, 217335, 217336, 217337, 217338) c6( )c7(fq); #c1(FBX017) c2(NP_680474) c3(F-box only protein 17 isoform 1) c4(242283) c5(242284, 242285, 242286, 242287, 242288, 242289, 242290) c6( ) c7(fq); #c1(FBX018) c2(NP_001245381) c3(F-box only protein 18 isoform 3) c4(93365) c5(93366, 93367, 93368, 93369, 93370) c6( ) c7(aW, Y, aKO, K, aT); #c1(FBX018) c2(NP_001245382) c3(F-box only protein 18 isoform 4) c4( )c5(93371) c6( )c7(aW, Y, aKO, K, aT); #c1(FBX018) c2(NP_116196) c3(F-box only protein 18 isoform 1) c4(225699) c5(225700, 225701, 225702, 225703, 225704, 225705) c6( ) c7(aW, Y, aKO, K, aT); #c1(FBX018) c2(NP_835363) c3(F-box only protein 18 isoform 2) c4(253476) c5(253477, 253478, 253479, 253480, 253481, 253482, 253483, 253484) c6( )c7(aW, Y, aKO, K, aT); #c1(FBX018) c2(XP_011518051) c3(F-box only protein 18 isoform X2) c4(312089) c5(312090, 312091, 312092, 312093, 312094, 312095, 312096, 312097) c6( )c7(aW, Y, aKO, K, aT); #c1(FBX028) c2(NP_001129587) c3(F-box only protein 28 isoform b) c4(51948) c5(51949, 51950, 51951, 51952, 51953, 51954) c6( )c7(aW, T, aT); #c1(FBX028) c2(NP_055991) c3(F-box only protein 28 isoform a) c4(184987) c5(184988, 184989, 184990, 184991, 184992, 184993) c6( ) c7(aW, T, aT); #c1(FBX03) c2(NP_036307) c3(F-box only protein 3 isoform 1) c4(172903) c5(172904, 172905, 172906, 172907, 172908, (72909, 172910) c6( )c7(bdR, an); #c1(FBX03) c2(NP_208385) c3(F-box only protein 3 isoform 2) c4(228553, 228554) c5(228555, 228556, 228557, 228558, 228559, 228560, 228561, 228562, 228563, 228564, 228565) c6( )c7(bdR, an); #c1(FBX030) c2(XP_005267216) c3(F-box only protein 30 isoform X1) c4(280829) c5(280830, 280831, 280832, 280833, 280834, 280835) c6( ) c7(ci); #c1(FBX031) c2(NP_001269612) c3(F-box only protein 31 isoform 2) c4( )c5(102212) c6( )c7(aW, aT, K, nA, rz, it, ad); #c1(FBX031) c2(NP_079011) c3(F-box only protein 31 isoform 1) c4(216545) c5(216546, 216547, 216548, 216549, 216550, 216551, 216552) c6( )c7(aW, aT, K, nA, rz, it, ad); #c1(FBX032) c2(NP_001229392) c3(F-box only protein 32 isoform 3) c4(84346) c5(84347, 84348, 84349, 84350, 84351, 84352) c6( )c7(ID, ht, aj, ci, C, aY, J, no, oj, cy, bl, K, hu, brd, li, ly, t, Bu, lx, c, at, aS, m, it); #c1(FBX032) c2(NP_478136) c3(F-box only protein 32 isoform 1) c4(230979) c5(230980, 230981, 230982, 230983, 230984, 230985) c6( )c7(ID, ht, aj, ci, C, aY, J, no, oj, cy, bl, K, hu, brQ, li, ly, t, Bu, lx, c, at, aS, m, it); #c1(FBX033) c2(NP_976046) c3(F-box only protein 33) c4(262879) c5(262880, 262881, 262882, 262883, 262884, 262885, 262886) c6( )c7(bM, dY, oe, EQ, aHP, cm, aHQ, cd); #c1(FBX038) c2(NP_001258652) c3(F-box only protein 38 isoform c) c4(96278) c5(96279, 96280, 96281, 96282, 96283, 96284, 96285, 96286, 96287, 96288) c6( )c7(Oq, brR, bL, A, no, cz, j, y); #c1(FBX038) c2(NP_110420) c3(F-box only protein 38 isoform a) c4(220238) c5(220239, 220240, 220241, 220242, 220243, 220244) c6( )c7(Oq, brR, bL, A, no, cz, j, y); #c1(FBX038) c2(XP_005268570) c3(F-box only protein 38 isoform X1) c4(281922) c5(281923, 281924, 281925, 281926, 281927, 281928) c6( )c7(Oq, brR, bL, A, no, cz, j, y); #c1(FBX04) c2(NP_036308) c3(F-box only protein 4 isoform 1) c4(172911) c5(172912, 172913, 172914, 172915, 172916, 172917) c6( )c7(T, Y); #c1(FBX04) c2(NP_277019) c3(F-box only protein 4 isoform 2) c4(228738) c5(228739, 228740, 228741, 228742, 228743, 228744) c6( )c7(T, Y); #c1(FBX040) c2(NP_057382) c3(F-box only protein 40) c4(190477) c5(190478, 190479, 190480, 190481, 190482, 190483) c6( )c7(bN); #c1(FBX047) c2(XP_011523166) c3(F-box only protein 47 isoform X1) c4(317697) c5(317698, 317699, 317700, 3/7701, 317702, 317703) c6( )c7(rP); #c1(FBX05) c2(NP_001135994) c3(F-box only protein 5 isoform b) c4( )c5(53931, 53932, 53933, 53934, 53935) c6( )c7(T, rz, aW, aT, K, nA, t, it, B, ad, bA); #c1(FBX05) c2(NP_036309) c3(F-box only protein 5 isoform a) c4(172918) c5(172919, 172920, 172921, 172922, 172923, 172924) c6( )c7(T, rz, aW, aT, K, nA, t, it, B, ad, bA); #c1(FBX05) c2(XP_011534051) c3(F-box only protein 5 isoform X1) c4(330224) c5(330225) c6( )c7(T, rz, aW, aT, K, nA, t, it, B, ad, bA); #c1(FBX07) c2(NP_001028196) c3(F-box only protein 7 isoform 2) c4(30353) c5(30354, 30355, 30356, 30357, 30358, 30359) c6( )c7(T, brS, U, kH, GC, ci, brT, an); #c1(FBX07) c2(NP_001244919) c3(F-box only protein 7 isoform 3) c4( )c5(92330, 92331, 92332, 92333, 92334) c6( )c7(T, brS, U, kH, GC, ci, brT, an); #c1(FBX07) c2(NP_036311) c3(F-box only protein 7 isoform 1) c4(172925, 172926) c5(172927, 172928, 172929, 172930, 172931, 172932, 172933, 172934, 172935, 172936, 172937) c6( )c7(T, brS, U, kH, GC, ci, brT, an); #c1(FBX08) c2(NP_036312) c3(F-box only protein 8) c4(172938, 172939, 172940) c5(172941, 172942, 172943, 172944, 172945, 172946, 172947, 172948, 172949, 172950, 172951, 172952, 172953, 172954, 172955, 172956, 172957, 172958) c6( )c7(bM, bP, c, vA, ff, aq, en, iR, cm, ae, B, op, ad); #c1(FBX09) c2(NP_036479) c3(F-box only protein 9 isoform 1) c4(174154) c5(174155, 174156, 174157, 174158, 174159, 174160, 174161) c6( )c7(fd); #c1(FBX09) c2(NP_258441) c3(F-box only protein 9 isoform 2) c4(228704, 228705) c5(228706, 228707, 228708, 228709, 228710, 228711, 228712, 228713, 228714) c6( )c7(fd); #c1(FBX09) c2(NP_258442) c3(F-box only protein 9 isoform 3) c4(228715, 228716, 228717) c5(228718, 228719, 228720, 228721, 228722, 228723, 228724, 228725, 228726, 228727, 228728, 228729, 228730, 228731, 228732, 228733, 228734, 228735, 228736, 228737) c6( )c7(fd); #c1(FBXW11) c2(NP_036432) c3(F-box/WD repeat-containing protein 11 isoform C) c4(173815) c5(173816, 173817, 173818, 173819, 173820, 173821) c6( )c7(bF, T, ft, eG, aE, atk, bkF, eH, Hn, h, aF, zG, ci, ZW, eF, cm, eL, B, afb, qH); #c1(FBXW11) c2(NP_387448) c3(F-box/WD repeat-containing protein 11 isoform B) c4(228949, 228950) c5(228951, 228952, 228953, 228954, 228955, 228956, 228957, 228958, 228959, 228960, 228961) c6( )c7(bF, T, ft, eG, aE, atk, bkF, eH, Hn, h, aF, zG, ci, ZW, eF, cm, eL, B, afb, qH); #c1(FBXW11) c2(NP_387449) c3(F-box/WD repeat-containing protein 11 isoform A) c4(228962) c5(228963, 228964, 228965, 228966, 228967, 228968) c6( )c7(bF, T, ft, eG, aE, atk, bkF, eH, Hn, h, aF, zG, ci, ZW, eF, cm, eL, B, afb, qH); #c1(FBXW11) c2(XP_005265913) c3(F-box/WD repeat-containing protein fl isoform X3) c4( )c5(279842) c6( )c7(bF, T, ft, eG, aE, atk, bkF, eH, Hn, h, aF, zG, ci, ZW, eF, cm, eL, B, afb, qH); #c1(FBXW4) c2(NP_071322) c3(F-box/WD repeat-containing protein 4) c4(210949, 210950, 210951) c5(210952, 210953, 210954, 210955, 210956, 210957, 210958, 210959, 210960, 210961, 210962, 210963, 210964, 210965, 210966, 210967, 210968, 210969, 210970, 210971, 210972, 210973, 210974, 210975) c6( )c7(bv, arB, d, arA, aW, bs, aT, bw, m, br, bu, t, bt, atb); #c1(FBXW4) c2(XP_005270110) c3(F-box/WD repeat-containing protein 4 isoform X1) c4(283110) c5( )c6( )c7(bv, arB, d, arA, aW, bs, aT, bw, m, br, bu, t, bt, atb); #c1(FBXW7) c2(NP_060785) c3(F-box/WD repeat-containing protein 7 isoform 2) c4(197795) c5(197796, 197797, 197798, 197799, 197800, 197801) c6( )c7(ph, brU, T, am, qo, aC, ht, jN, jh, aA, n, ci, q, ad, er, hX, eA, eJ, iu, x, fx, aE, aaU, dE, qK, dV, kH, aT, rD, h, hn, dL, K, hu, brV, KV, ae, vO, cp, Qe, le, d, s, dH, iU, aW, Y, ff, eZ, r, af, en, cl, g, t, eF, awi, dr, ck, es, bF, BU, vD, eM, aiM, cj, pP, hL, CD, at, lF, m, k, cP, pw, it, B, jr); #c1(FBXW7) c2(XP_011530387) c3(F-box/WD repeat-containing protein 7 isoform X1) c4(326227, 326228) c5(326229, 326230, 326231, 326232, 326233, 326234) c6( )c7(ph, brU, T, am, qo, aC, ht, jN, jh, aA, n, ci, q, ad, er, hX, eA, eJ, iu, x, fx, aE, aaU, dE, qK, dV, kH, aT, rD, h, hn, dL, K, hu, brV, KV, ae, vO, cp, De, le, d, s, dH, iU, aW, Y, ff, eZ, r, af, en, cl, g, t, eF, awi, dr, ck, es, bF, BU, vD, eM, aiM, cj, pP, hL, CD, at, lF, m, k, cP, pw, it, B, jr); #c1(FBXW7) c2(XP_011530389) c3(F-box/WD repeat-containing protein 7 isoform X2) c4(326235) c5(326236, 326237, 326238, 326239, 326240, 326241) c6( )c7(ph, brl, T, am, qo, aC, ht, jN, jh, aA, n, ci, q, ad, er, hX, eA, eJ, iu, x, fx, aE, aaU, dE, qK, dV, kH, aT, rO, h, hn, dL, K, hu, brV, KV, ae, vO, cp, De, le, d, s, dH, il, aW, Y, ff, eZ, r, aF, en, cl, g, t, eF, awi, dr, ck, es, bF, BU, vD, eM, aiM, cj, pP, hL, CD, at, lF, m, k, cP, pw, it, B, jr); #c1(FBXW7) c2(XP_011530390) c3(F-box/WD repeat-containing protein 7 isoform X3) c4( )c5(326242, 326243, 326244, 326245, 326246, 326247) c6( )c7(ph, brU, T, am, qo, aC, ht, jN, jh, aA, n, ci, q, ad, er, hX, eA, eJ, iu, x, fx, aE, aaU, dE, qK, dV, kH, aT, rO, h, hn, dL, K, hu, brV, KV, ae, vO, cp, Qe, le, d, s, dH, iU, aW, Y, ff, eZ, r, aF, en, cl, g, t, eF, awi, dr, ck, es, bF, BU, vD, eM, aiM, cj, pP, hL, CD, at, lF, m, k, cP, pw, it, B, jr); #c1(FBXW8) c2(NP_036306) c3(F-box/WD repeat-containing protein 8 isoform 2) c4(172892) c5(172893, 172894, 172895, 172896, 172897, 172898, 172899, 172900, 172901, 172902) c6( )c7(nJ, aE); #c1(FBXW8) c2(NP_699 (79) c3(F-box/WD repeat-containing protein 8 isoform 1) c4(245806) c5(245807, 245808, 245809, 245810, 245811, 245812) c6( )c7(nJ, aE); #c1(FCAR) c2(NP_001991) c3(immunoglobulin alpha Fc receptor isoform a precursor) c4(122967, (22968) c5(122969, 122970, 122971, 122972, 122973, 122974, 122975, 122976, 122977, 122978, 122979, 122980) c6( )c7(aFe, fR, eR, e, aw, fd, j, C, Tz, F, ey, eu, eZ, brW, iV, pb, ac, hc, afb, uz, fl, y); #c1(FCAR) c2(NP_579803) c3(immunoglobulin alpha Fc receptor isoform b precursor) c4(234620) c5(234621, 234622, 234623, 234624, 234625, 234626) c6( )c7(aFe, fR, eR, e, aw, fd, j, C, Tz, F, ey, eu, eZ, brW, iV, pb, ac, hc, aFb, uz, fl, y); #c1(FCAR) c2(NP_579805) c3(immunoglobulin alpha Fc receptor isoform c precursor) c4(234627) c5(234628, 234629, 234630, 234631, 234632, 234633) c6( )c7(aFe, fR, eR, e, aw, fd, j, C, Tz, F, ey, eu, eZ, brW, iV, pb, ac, hc, aFb, uz, fl, y); #c1(FCAR) c2(NP_579806) c3(immunoglobulin alpha Fc receptor isoform d) c4(234634) c5(234635, 234636, 234637, 234638, 234639, 234640) c6( )c7(aFe, fR, eR, e, aw, fd, j, C, Tz, F, ey, eu, eZ, brW, iV, pb, ac, hc, aFb, uz, fL, y); #c1(FCAR) c2(NP_579807) c3(immunoglobulin alpha Fc receptor isoform e) c4(234641) c5(234642, 234643, 234644, 234645, 234646, 234647) c6( )c7(aFe, fR, eR, e, aw, fd, j, C, Tz, F, ey, eu, eZ, brW, iV, pb, ac, hc, aFb, uz, fl, y); #c1(FCAR) c2(NP_579808) c3 (immunoglobulin alpha Fc receptor isoform f) c4(234648) c5(234649, 234650, 234651, 234652, 234653, 234654) c6( )c7(aFe, fR, eR, e, aw, fd, j, C, Tz, F, ey, eu, eZ, brW, iV, pb, ac, hc, aFb, uz, fl, y); #c1(FCAR) c2(NP_579811) c3(immunoglobulin alpha Fc receptor isoform g) c4(234655) c5(234656, 234657, 234658, 234659, 234660, 234661) c6( )c7(aFe, fR, eR, e, aw, fd, j, C, Tz, F, ey, eu, eZ, brW, iV, pb, ac, hc, aFb, uz, fl, y); #c1(FCAR) c2(NP_579812) c3(immunoglobulin alpha Fc receptor isoform h) c4(234662) c5(234663, 234664, 234665, 234666, 234667, 234668) c6( )c7(aFe, fR, eR, e, aw, fd, j, C, Tz, F, ey, eu, eZ, brW, iV, pb, ac, hc, aFb, uz, fl, y); #c1(FCER1A) c2(NP_001992) c3(high affinity immunoglobulin epsilon receptor subunit alpha precursor) c4(122981) c5(122982, 122983, 122984, 122985, 122986, 122987) c6( )c7(ck, bM, cT, Dw, aW, fS, e, eE, JM, aT, bm, uz, Ds, fe, j, Jx, ve, Vk, eA); #c1(FCER1G) c2(NP_004097) c3(high affinity immunoglobulin epsilon receptor subunit gamma precursor) c4(143527, 143528) c5(143529, 143530, 143531, 143532, 143533, 143534, 143535, 143536, 143537, 143538, 143539, 143540) c6( )c7(bM, il, e, fS, eZ, ah, aak, eE, ip); #c1(FCER2) c2(NP_001193948) c3(low affinity immunoglobulin epsilon Fc receptor isoform b) c4(83310) c5(83311, 83312, 83313, 83314, 83315, 83316) c6( )c7(bM, Dw, fM, iW, Ju, eX, jh, fd, v, pM, ai, zn, De, F, bW, brX, hD, h, Ds, hn, eV, an, pk, XI, A, sp, zb, wc, BG, aoD, eE, asE, yR, Cv, ck, cT, il, z, kz, cj, hh, y, Ze); #c1(FCER2) c2(NP_001207429) c3(low affinity immunoglobulin epsilon Fc receptor isoform a) c4(83711) c5(83712, 83713, 83714, 83715, 83716, 83717) c6( )c7(bM, Dw, fM, iW, Ju, eX, jh, fd, v, pM, ai, zn, De, F, bW, brX, hD, h, Ds, hn, eV, an, pk, XI, A, sp, zb, wc, BG, aoD, eE, asE, yR, Cv, ck, cT, il, z, kz, cj, hh, y, Ze); #c1(FCGBP) c2(NP_003881) c3(IgGFc-binding protein precursor) c4(14/779, 141780) c5(141781, 141782, 141783, 141784, 141785, 141786, 141787, 14/788) c6(141789, 141790) c7(qJ, hd, AS, aA, ae, AN, ad); #c1(FCGBP) c2(XP_011547114) c3(IgGFc-binding protein isoform X2) c4( ) c5(345869, 345870, 345871, 345872, 345873, 345874, 345875) c6( )c7(qJ, hd, AS, aA, ae, AN, ad); #c1(FCGR2A) c2(NP_001129691) c3(low affinity immunoglobulin gamma Fc region receptor II-a isoform 1 precursor) c4(52208) c5(52209, 52210, 52211, 52212, 52213, 52214) c6( )c7(oO, bM, fR, e, fd, bh, aDI, Tz, dY, dV, M, DY, hH, azx, UC, aiV, aW, asD, pD, ve, cm, bqd, cj, Zn, k, eg, aFf, dD, fN, ez, uE, bsa, zo, ai, brY, gd, h, nm, dL, DU, ey, A, fe, cM, Wt, dB, JM, uz, fL, Pr, fM, qd, aA, bcE, OY, j, C, V, pd, q, zT, jZ, sl, aT, kf, aFm, lM, ae, eu, hM, Ue, bsb, bg, apk, r, aDm, pb, brZ, cT, ac, ig, AH, bsc, dT, wR, CZ, dy, fD, bt, ao, gV, id, aFe, qb, aZ, iW, jh, ah, eq, atK, mC, ad, wD, aZp, afm, oW, Os, hD, lQ, vO, sj, wc, bps, eZ, brp, aF, hp, S, iV, aYt, LB, aFb, aS, XO, ME, lf, ne, y); #c1(FCGR2A) c2(NP_067674) c3(low affinity immunoglobulin gamma Fc region receptor Il-a isoform 2 precursor) c4(209261) c5(209262, 209263, 209264, 209265, 209266, 209267, 209268, 209269, 209270) c6( )c7(oO, bM, fR, e, fd, bh, aDI, Tz, dY, dV, M, DY, hH, azx, UC, aiV, aW, asD, pD, ve, cm, bqd, cj, Zn, k, eg, aFf, dD, fN, ez, uE, bsa, zo, ai, brY, gd, h, nm, dL, DU, ey, A, fe, cM, Wt, dB, JM, uz, fl, Pr, fM, qd, aA, bcE, OY, j, C, V, pd, q, zT, jZ, sl, aT, kf, aFm, lM, ae, eu, hM, Ue, bsb, bg, apk, r, aDm, pb, brZ, cT, ac, ig, AH, bsc, dT, wR, CZ, dy, fD, bt, ao, gV, id, aFe, qb, aZ, iW, jh, ah, eq, atK, mC, ad, wD, aZp, afm, oW, Os, hD, lQ, vO, sj, wc, bps, eZ, brp, aF, hp, S, iV, aYt, LB, aFb, aS, XO, ME, lf, ne, y); #c1(FCGR2B) c2(NP_001002273) c3(low affinity immunoglobulin gamma Fc region receptor Il-b isoform 2 precursor) c4( )c5(19544, 19545, 19546, 19547, 19548, 19549) c6( )c7(oO, gV, bM, fM, id, aZ, jh, fd, cR, ol, BH, j, ah, bh, mC, ad, aDl, ai, x, aZp, afm, oz, jZ, oW, Os, aT, dV, h, ey, lQ, DY, ae, bsd, UC, bsb, Y, eZ, and, aDm, pD, VE, S, Wt, cT, y, yW, uz, dT, dy, k, fl, eg, XO, lf, bt, Pr, ME, hv); #c1(FCGR2B) c2(NP_001002274) c3(low affinity immunoglobulin gamma Fc region receptor Il-b isoform 3 precursor) c4(19550, 19551) c5(19552, 19553, 19554, 19555, 19556, 19557, 19558, 19559, 19560, 19561) c6( )c7(oO, gV, bM, fM, id, aZ, jh, fd, cR, ol, BH, j, ah, bh, mC, ad, aDI, ai, x, aZp, afm, oz, jZ, oW, Os, aT, dV, h, ey, lQ, DY, ae, bsd, UC, bsb, Y, eZ, and, aDm, pD, VE, S, Wt, cT, y, yW, uz, dT, dy, k, fl, eg, XO, lf, bt, Pr, ME, hv); #c1(FCGR2B) c2(NP_001177757) c3(low affinity immunoglobulin gamma Fc region receptor II-b isoform 5) c4(72969) c5(72970, 72971, 72972, 72973, 72974, 72975, 72976, 72977, 72978, 72979) c6( )c7(oO, gV, bM, fM, id, aZ, jh, fd, cR, ol, BH, j, ah, bh, mC, ad, aDI, ai, x, aZp, afm, oz, jZ, OW, Os, aT, dV, h, ey, lQ, DY, ae, bsd, UC, bsb, Y, eZ, and, aDm, pD, VE, S, Wt, cT, y, yW, uz, dT, dy, k, fl, eg, XO, lf, bt, Pr, ME, hv); #c1(FCGR2B) c2(NP_003992) c3(low affinity immunoglobulin gamma Fc region receptor Il-b isoform 1 precursor) c4(142688) c5(142689, 142690, 142691, 142692, 142693, 142694, 142695, 142696, 142697, 142698) c6( )c7(oO, gV, bM, fM, id, aZ, jh, fd, cR, ol, BH, j, ah, bh, mC, ad, aDI, ai, x, aZp, afm, oz, jZ, oW, Os, aT, dV, h, ey, lQ, DY, ae, bsd, UC, bsb, Y, eZ, and, aDm, pD, VE, S, Wt, cT, y, yW, uz, dT, dy, k, fl, eg, XO, lf, bt, Pr, ME, hv); #c1(FCGR2B) c2(XP_011507594) c3(low affinity immunoglobulin gamma Fc region receptor Il-b isoform XI) c4(297499) c5(297500, 297501, 297502, 297503, 297504, 297505) c6( )c7(oO, gV, bM, fM, id, aZ, jh, fd, cR, ol, BH, j, ah, bh, mC, ad, aDI, ai, x, aZp, afm, oz, jZ, oW, Os, aT, dV, h, ey, lQ, DY, ae, bsd, UC, bsb, Y, eZ, and, aDm, pD, VE, S, Wt, cT, y, yW, uz, dT, dy, k, fl, eg, XO, lf, bt, Pr, ME, hv); #c1(FCGR3A) c2(NP_000560) c3(low affinity immunoglobulin gamma Fc region receptor III-A isoform a precursor) c4(12666, 12667) c5(12668, 12669, 12670, 12671, 12672, 12673, 12674, 12675, 12676, 12677, 12678) c6( )c7(bM, T, fR, ea, e, fd, bB, ci, aY, aDI, amW, Tz, dV, DY, hH, hm, aiV, aW, az, pD, g, ve, cm, ba, eM, xD, dc, Fh, cj, Zn, hh, fy, dD, aYZ, aw, fr, uE, aHj, ai, eW, gd, kH, bsg, h, eV, hn, DU, ey, afu, A, anP, aHW, CM, lb, fe, ax, Og, yq, bse, cM, Wt, asE, bsf, dB, gw, uz, eo, fl, Pr, fM, dA, eR, jx, eX, fZ, aA, hy, v, j, OY, C, bcE, aDO, V, aFv, q, aGT, zT, ld, aT, lM, ae, HM, iU, Y, bg, r, lo, en, zM, aER, iK, pb, Cv, pa, il, ig, hc, CZ, dy, fD, bt, vi, gV, aFe, Ke, ZI, ob, aD, iW, jh, ah, eq, mC, ad, wD, F, afm, ul, ue, hG, cW, ff, Of, eZ, brp, xH, hp, Fq, iV, dr, bE, aFb, XO, ME, lf, fh, y); #c1(FCGR3A) c2(NP_001121064) c3(low affinity immunoglobulin gamma Fc region receptor III-A isoform b precursor) c4(45120, 45121) c5(45122, 45123, 45124, 45125, 45126, 45127, 45128, 45129, 45130, 45131, 45132, 45133, 45134) c6( )c7(bM, T, fR, ea, e, fd, bB, ci, aY, aDI, amW, Tz, dV, DY, hH, hm, aiV, aW, az, pD, g, ve, cm, ba, eM, xD, dc, Fh, cj, Zn, hh, fy, dD, aYZ, aw, fr, uE, aHj, ai, eW, gd, kH, bsg, h, eV, hn, DU, ey, afu, A, anP, aHW, CM, lb, fe, ax, Og, yq, bse, cM, Wt, asE, bsf, dB, gw, uz, eo, fl, Pr, fM, dA, eR, jx, eX, fZ, aA, hy, v, j, OY, C, bcE, aDO, V, aFv, q, aGT, zT, ld, aT, lM, ae, hM, iU, Y, bg, r, lo, en, zM, aER, ik, pb, Cv, pa, il, ig, hc, CZ, dy, fD, bt, vi, gV, aFe, Ke, ZI, ob, aD, iW, jh, ah, eq, mC, ad, wD, F, afm, ul, ue, hG, cW, ff, Of, eZ, brp, xH, hp, Fq, iV, dr, bE, aFb, XO, ME, lf, fh, y); #c1(FCGR3A) c2(NP_001121065) c3 (low affinity immunoglobulin gamma Fc region receptor III-A isoform c precursor) c4( )c5(45135, 45136, 45137, 45138, 45139, 45140, 45141, 45142, 45143, 45144, 45145) c6( ) c7(bM, T, fR, ea, e, fd, bB, ci, aY, aDI, amW, Tz, dV, DY, hH, hm, aiV, aW, az, pD, g, ve, cm, ba, eM, xD, dc, Fh, cj, Zn, hh, fy, dD, aYZ, aw, fr, uE, aHj, ai, eW, gd, kH, bsg, h, eV, hn, DU, ey, afu, A, anP, aHW, CM, lb, fe, ax, Og, yq, bse, cM, Wt, asE, bsf, dB, gw, uz, eo, fl, Pr, fM, dA, eR, jx, eX, fZ, aA, hy, v, j, OY, C, bcE, aDO, V, aFv, q, aGT, zT, ld, aT, lM, ae, hM, iU, Y, bg, r, lo, en, zM, aER, iK, pb, Cv, pa, il, ig, hc, CZ, dy, fD, bt, vi, gV, aFe, Ke, ZI, ob, aD, iW, jh, ah, eq, mC, ad, wD, F, afm, ul, ue, hG, cW, ff, Of, eZ, brp, xH, hp, Fq, iV, dr, bE, aFb, XO, ME, lf, fh, y); #c1(FCGR3A) c2(NP_001121068) c3(low affinity immunoglobulin gamma Fc region receptor III-A isoform d precursor) c4(45146) c5(45147, 45148, 45149, 45150, 45151) c6( )c7(bM, T, fR, ea, e, fd, bB, ci, aY, aDI, amW, Tz, dV, DY, hH, hm, aiV, aW, az, pD, g, ve, cm, ba, eM, xD, dc, Fh, cj, Zn, hh, fy, dD, aYZ, aw, fr, uE, aHj, ai, eW, gd, kH, bsg, h, eV, hn, DU, ey, afu, A, anP, aHW, CM, lb, fe, ax, Og, yq, bse, cM, Wt, asE, bsf, dB, gw, uz, eo, fL, Pr, fM, dA, eR, jx, eX, fZ, aA, hy, v, j, OY, C, bcE, aDO, V, aFv, q, aGT, zT, ld, aT, lM, ae, hM, iU, Y, bg, r, lo, en, zM, aER, ik, pb, Cv, pa, il, ig, hc, CZ, dy, fD, bt, vi, gV, aFe, Ke, ZI, ob, aD, iW, jh, ah, eq, mC, ad, wD, F, afm, ul, ue, hG, cW, ff, Of, eZ, brp, xH, hp, Fq, iV, dr, bE, aFb, XO, ME, lf, fh, y); #c1(FCGR3B) c2(NP_000561) c3(low affinity immunoglobulin gamma Fc region receptor III-B isoform 2 precursor) c4(12679) c5(12680, 12681, 12682, 12683, 12684, 12685, 12686, 12687) c6( )c7(oO, bM, T, ea, e, fd, ci, Tz, amW, dV, DY, hH, hm, aiV, aW, az, pD, g, ve, cm, y, eM, dc, cj, dW, hh, fy, dD, aYZ, aw, fr, uE, aHj, ai, eW, kH, h, DU, ey, afu, anP, fJ, fe, Wt, asE, bsf, uz, eo, fl, Pr, dA, eR, jx, fZ, v, j, OY, C, aDO, V, aFv, zT, ld, aT, kf, aFm, hM, il, Y, bg, lo, en, zM, aER, pb, Cv, pa, hc, CZ, fD, bt, vi, gV, aFe, Ke, qb, aD, jh, ah, eq, mC, F, afm, oW, vO, ue, hG, ff, eZ, brp, xH, Fq, iV, LB, aFb, aS, ME, lf, fh, hv); #c1(FCGRT) c2(NP_001129491) c3(IgG receptor FcRn large subunit p51 precursor) c4(51625, 51626, 51627) c5(51628, 51629, 51630, 51631, 51632, 51633, 51634, 51635, 51636, 51637) c6( ) c7(bM, fM, dM, jh, V, ai, q, mC, x, xy, Bj, jk, kH, ey, DY, uM, A, d, bf, je, eZ, r, afq, hp, eE, Wt, ck, iM, lV, awM, dy, fL, k, ER, R, bC, bt, be, aLL); #c1(FCGRT) c2(XP_011524929) c3(IgG receptor FcRn large subunit p51 isoform X1) c4( )c5(319412) c6( )c7(bM, fM, dM, jh, V, ai, q, mC, x, xy, Bj, jk, kH, ey, DY, uM, A, d, bf, je, eZ, r, afq, hp, eE, Wt, ck, iM, lV, awM, dy, FL, k, ER, R, bC, bt, be, aLL); #c1(FCGRT) c2(XP_011524930) c3(IgG receptor FcRn large subunit p51 isoform X3) c4( )c5(319413, 319414, 319415, 319416, 319417, 319418, 319419, 319420, 319421, 319422, 319423) c6( )c7(bM, fM, dM, jh, V, ai, q, mC, x, xy, Bj, jk, kH, ey, DY, uM, A, d, bf, je, eZ, r, afq, hp, eE, Wt, ck, iM, lV, awM, dy, fL, k, ER, R, bC, bt, be, aLL); #c1 (FCH01) c2(NP_001154829) c3(FCH domain only protein 1 isoform a) c4(61/70) c5(61171, 61172, 61173, 61174, 61175, 61176) c6( )c7(De); #c1(FCH01) c2(NP_055937) c3(FCH domain only protein 1 isoform b) c4(184533, 184534) c5(184535, 184536, 184537, 184538, 184539, 184540, 184541, 184542, 184543, 184544, 184545, 184546, 184547, 184548, 184549, 184550, 184551, 184552, 184553, 184554, 184555, 184556, 184557, 184558, 184559, 184560, 184561, 184562) c6( )c7(ce); #c1(FCH02) c2(NP_001139504) c3 (FCH domain only protein 2 isoform b) c4(59442) c5(59443, 59444, 59445, 59446, 59447, 59448) c6( )c7(ad); #c1(FCH02) c2(NP_620137) c3(FCH domain only protein 2 isoform a) c4(237331, 237332, 237333) c5(237334, 237335, 237336, 237337, 237338, 237339, 237340, 237341, 237342, 237343, 237344, 237345, 237346, 237347, 237348, 237349, 237350, 237351) c6( )c7(ad); #c1(FCHSD2) c2(NP_055639) c3(FCH and double SH3 domains protein 2) c4(182439) c5(182440, 182441, 182442, 182443, 182444, 182445, 182446, 182447, 182448, 182449, 182450, 182451, 182452, 182453, 182454, 182455, 182456, 182457) c6( )c7(ck, bt); #c1(FCHSD2) c2(XP_011543711) c3(FCH and double SH3 domains protein 2 isoform X1) c4(342785) c5( )c6( )c7(ck, bt); #c1(FCHSD2) c2(XP_011543714) c3(FCH and double SH3 domains protein 2 isoform X4) c4(342786) c5( )c6( )c7(ck, bt); #c1(FCN1) c2(NP_001994) c3(ficolin-1 precursor) c4(122988, 122989, 122990) c5(122991, 122992, 122993, 122994, 122995, 122996, 122997, 122998, 122999, 123000, 123001, 123002, 123003, 123004, 123005, 123006, 123007, 123008) c6( )c7(y, wz, aYt, aa); #c1(FCN2) c2(NP_004099) c3(ficolin-2 isoform a precursor) c4(143541) c5(143542, 143543, 143544, 143545, 143546, 143547) c6( )c7(oO, bM, T, hl, JZ, jx, oH, fw, aR, aEl, hX, AW, ga, afm, hb, afn, dV, Os, bsi, hH, vO, af, A, Cw, Y, eH, Pg, eZ, lo, as, ef, t, acD, cm, KT, WO, ac, ig, aDs, WP, U, m, y, xg, bsh, ek); #c1(FCN2) c2(NP_056652) c3(ficolin-2 isoform b precursor) c4(188105) c5(188106, 188107, 188108, 188109, 188110, 188111) c6( )c7(oO, bM, T, hl, JZ, jx, OH, fw, aR, aEl, hX, AW, ga, afm, hb, afn, dV, Os, bsi, hH, vO, af, A, Cw, Y, eH, Pg, eZ, lo, as, ef, t, acD, cm, kT, WO, ac, ig, aDs, WP, U, m, y, xg, bsh, ek); #c1(FCRL1) c2(NP_001152869) c3(Fc receptor-like protein 1 isoform 2 precursor) c4(60264) c5(60265, 60266, 60267, 60268, 60269, 60270) c6( )c7(x, Y, k, cj); #c1(FCRL1) c2(NP_001152870) c3(Fc receptor-like protein 1 isoform 3 precursor) c4(60271) c5(60272, 60273, 60274, 60275, 60276, 60277) c6( )c7(x, Y, k, cj); #c1(FCRL1) c2(NP_443170) c3(Fc receptor-like protein 1 isoform 1 precursor) c4(229716) c5(229717) c6( )c7(x, Y, k, cj); #c1(FCRL1) c2(XP_011507429) c3(Fc receptor-like protein 1 isoform X3) c4( ) c5(297352, 297353, 297354, 297355, 297356) c6( )c7(x, Y, k, cj); #c1(FCRL2) c2(NP_001152960) c3(Fc receptor-like protein 2 isoform 2 precursor) c4(60278) c5(60279, 60280, 60281, 60282, 60283, 60284) c6( )c7(cj); #c1(FCRL2) c2(NP_110391) c3(Fc receptor-like protein 2 isoform 1 precursor) c4(219989) c5(219990, 219991, 219992, 219993, 219994, 219995) c6( )c7(cj); #c1(FCRL2) c2(XP_006711598) c3(Fc receptor-like protein 2 isoform X7) c4( )c5(286165, 286166, 286167, 286168, 286169, 286170, 286171, 286172, 286173, 286174, 286175, 286176) c6( )c7(cj); #c1(FCRL2) c2(XP_011508278) c3(Fc receptor-like protein 2 isoform X3) c4(298972, 298973) c5( )c6( )c7(cj); #c1(FCRL2) c2(XP_011508282) c3(Fc receptor-like protein 2 isoform X8) c4(298974) c5(298975, 298976, 298977, 298978, 298979, 298980) c6( )c7(cj); #c1(FCRL3) c2(XP_006711208) c3(Fc receptor-like protein 3 isoform X1) c4(285771, 285772, 285773) c5(285774, 285775, 285776, 285777, 285778, 285779, 285780, 285781, 285782, 285783, 285784, 285785, 285786, 285787, 285788, 285789, 285790, 285791, 285792, 285793, 285794, 285795) c6( )c7(bM, hz, cl, OY, bh, V, ai, o, dP, aT, hD, hH, yF, A, zQ, mR, HA, eZ, fe, OC, eU, OW, Lj, cj, ol, y); #c1(FCRL4) c2(NP_112572) c3(Fc receptor-like protein 4 precursor) c4(221435) c5(221436, 221437, 221438, 221439, 221440, 221441) c6( )c7(bsj, T, cj, ac, aoH); #c1(FCRL4) c2(XP_011508336) c3(Fc receptor-like protein 4 isoform XI) c4( )c5(299022) c6( )c7(bsj, T, cj, ac, aoH); #c1(FCRL5) c2(NP_112571) c3(Fc receptor-like protein 5 isoform 1 precursor) c4(221418, 221419, 221420, 221421, 221422) c5(221423, 221424, 221425, 221426, 221427, 221428, 221429, 221430, 221431, 221432, 221433, 221434) c6( )c7(T, mR, jh, cj, hD, zn); #c1(FCRL5) c2(XP_011508332) c3(Fc receptor-like protein 5 isoform X1) c4(298997) c5(298998, 298999, 299000, 299001, 299002, 299003) c6( )c7(T, mR, jh, cj, hD, zn); #c1(FCRL5) c2(XP_011508335) c3(Fc receptor-like protein 5 isoform X4) c4( )c5(299004, 299005, 299006, 299007, 299008, 299009, 299010, 299011, 299012, 299013, 299014, 299015, 299016, 299017, 299018, 299019, 299020, 299021) c6( )c7(T, mR, jh, cj, hD, zn); #c1(FCRL6) c2(NP_001271146) c3(Fc receptor-like protein 6 isoform 2) c4( )c5(103467, 103468, 103469, 103470, 103471, 103472) c6( )c7(dD, ea, eZ, h, wR, eq, bN); #c1(FCRL6) c2(XP_005245185) c3(Fc receptor-like protein 6 isoform X1) c4(266349) c5( )c6( )c7(dD, ea, eZ, h, wR, eq, bN); #c1(FCRL6) c2(XP_005245188) c3 (Fc receptor-like protein 6 isoform X5) c4(266350) c5(266351, 266352, 266353, 266354, 266355, 266356) c6( )c7(dD, ea, eZ, h, wR, eq, bN); #c1(FCRL6) c2(XP_006711355) c3 (Fc receptor-like protein 6 isoform X4) c4(285935) c5( )c6( )c7(dD, ea, eZ, h, wR, eq, bN); #c1(FCRL6) c2(XP_011507782) c3(Fc receptor-like protein 6 isoform X2) c4( )c5(297845, 297846, 297847, 297848, 297849, 297850) c6( )c7(dD, ea, eZ, h, wR, eq, bN); #c1(FCRLA) c2(NP_001171795) c3(Fc receptor-like A isoform 1 precursor) c4(71662, 71663, 71664) c5(71665, 71666, 71667, 71668, 71669, 71670, 71671, 71672, 71673, 71674, 71675, 71676, 71677, 71678, 71679, 71680, 71681, 71682, 71683, 71684, 71685, 71686, 71687, 71688) c6(71689, 71690, 71691, 71692, 71693, 71694) c7(T, fM, Y, cj, hD, ey, cm, y); #c1(FCRLA) c2(NP_001171796) c3(Fc receptor-like A isoform 3 precursor) c4(71695, 71696) c5(71697, 71698, 71699, 71700, 71701, 71702, 71703, 71704, 71705, 71706, 71707, 7/708, 71709, 71710, 71711, 71712) c6(71713, 71714, 71715, 71716) c7(T, fM, Y, cj, hD, ey, cm, y); #c1(FCRLA) c2(NP_001171799) c3 (Fc receptor-like A isoform 4 precursor) c4(7/7/7, 71718) c5(71719, 71720, 71721, 71722, 71723, 71724, 71725, 71726, 71727, 71728, 71729, 71730, 71731, 71732, 71733, 71734) c6(71735, 71736, 71737, 71738) c7(T, fM, Y, cj, hD, ey, cm, y); #c1(FCRLA) c2(NP_001171800) c3(Fc receptor-like A isoform 7 precursor) c4(71739) c5(71740, 71741, 71742, 71743, 71744, 71745, 71746, 71747, 71748) c6(71749, 71750) c7(T, fM, Y, cj, hD, ey, cm, y); #c1(FCRLA) c2(NP_001171801) c3(Fc receptor-like A isoform 5 precursor) c4(71751) c5(71752, 71753, 71754, 71755, 71756, 71757, 71758, 71759) c6(71760, 71761) c7(T, fM, Y, cj, hD, ey, cm, y); #c1(FCRLA) c2(NP_001171802) c3(Fc receptor-like A isoform 6 precursor) c4(71762, 71763) c5(71764, 71765, 71766, 7/767, 71768, 71769, 71770, 71771, 71772, 71773, 71774, 71775, 71776, 71777, 71778, 71779) c6(71780, 71781, 71782, 71783) c7(T, fM, Y, cj, hD, ey, cm, y); #c1(FCRLA) c2(NP_116127) c3(Fc receptor-like A isoform 2 precursor) c4(225447) c5(225448, 225449, 225450, 225451, 225452, 225453, 225454, 225455) c6(225456, 225457) c7(T, fM, Y, cj, hD, ey, cm, y); #c1(FCRLA) c2(XP_011508368) c3(Fc receptor-like A isoform X4) c4(299052) c5(299053, 299054, 299055, 299056, 299057, 299058, 299059, 299060) c6(299061, 299062) c7(T, fM, Y, cj, hD, ey, cm, y); #c1(FCRLB) c2(NP_001275758) c3(Fc receptor-like B isoform 2 precursor) c4(107242) c5(107243, 107244, 107245, 107246, 107247, 107248) c6( )c7(ac, e, aw, cj, ey, j, cm, eu); #c1(FCRLB) c2(NP_001275759) c3(Fc receptor-like B isoform 3 precursor) c4(107249) c5(107250, 107251, 107252, 107253, 107254, 107255) c6( ) c7(ac, e, aw, cj, ey, j, cm, eu); #c1(FCRLB) c2(NP_001275760) c3(Fc receptor-like B isoform 4 precursor) c4(107256) c5(107257, 107258, 107259, 107260, 107261, 107262) c6( )c7(ac, e, aw, cj, ey, j, cm, eu); #c1(FCRLB) c2(NP_001275761) c3(Fc receptor-like B isoform 5 precursor) c4(107263) c5(107264, 107265, 107266, 107267, 107268, 107269) c6( )c7(ac, e, aw, cj, ey, j, cm, eu); #c1(FCRLB) c2(XP_011507495) c3(Fc receptor-like B isoform X1) c4(297393) c5(297394, 297395, 297396, 297397, 297398, 297399, 297400, 297401, 297402, 297403) c6( )c7(ac, e, aw, cj, ey, j, cm, eu); #c1(FDFT1) c2(NP_001274672) c3(squalene synthase isoform 1) c4(106610, 106611, 106612, 106613, 106614, 106615) c5(106616, 106617, 106618, 106619, 106620, 106621, 106622, 106623, 106624, 106625, 106626, 106627, 106628, 106629, 106630, 106631, 106632, 106633, 106634, 106635, 106636) c6( )c7(ae, T, mO, cH, iW, e, an, dN, bsk, cu, v, acl, aYT, ei, dQ, g, bz, B, ad, jb); #c1(FDFT1) c2(NP_001274678) c3(squalene synthase isoform 2) c4( )c5(106637, 106638, 106639, 106640, 106641, 106642, 106643, 106644, 106645, 106646) c6( )c7(ae, T, mO, cH, iW, e, an, dN, bsk, cu, v, acl, aYT, ei, dQ, g, bz, B, ad, jb); #c1(FDFT1) c2(NP_001274680) c3(squalene synthase isoform 4) c4( )c5(106647, 106648, 106649, 106650, 106651, 106652, 106653, 106654, 106655, 106656, 106657, 106658, 106659) c6( )c7(ae, T, mO, cH, iW, e, an, dN, bsk, cu, v, acl, aYT, ei, Qe, g, bz, B, ad, jb); #c1(FDPS) c2(NP_001129293) c3 (farnesyl pyrophosphate synthase isoform a) c4(51377) c5(51378, 51379, 51380, 51381, 51382, 51383, 51384, 51385, 51386, 51387) c6( )c7(T, aE, af, up, bE, FV, X, U, r, bB, cd, v, K, eF, q, bA); #c1(FDPS) c2(NP_001129294) c3(farnesyl pyrophosphate synthase isoform b) c4(51388) c5(51389, 51390, 51391, 51392, 51393, 51394) c6( )c7(T, aE, af, up, bE, FV, X, U, r, bB, cd, v, K, eF, q, bA); #c1(FDPS) c2(NP_001229754) c3(farnesyl pyrophosphate synthase isoform c) c4( )c5(85112) c6( )c7(T, aE, af, up, bE, FV, X, U, r, bB, cd, v, K, eF, q, bA); #c1(FDX1) c2(NP_004100) c3(adrenodoxin, mitochondrial precursor) c4(143548) c5(143549, 143550, 143551, 143552, 143553, 143554) c6( )c7(ar, gk, al, jN, v, nJ, B, hX); #c1(FDX1L) c2(NP_001026904) c3(adrenodoxin-like protein, mitochondrial precursor) c4(29955, 29956) c5(29957, 29958, 29959, 29960, 29961, 29962, 29963, 29964, 29965, 29966, 29967, 29968) c6( )c7(aNG, oj); #c1(FDXR) c2(NP_001244941) c3(NADPH:adrenodoxin oxidoreductase, mitochondrial isoform 3 precursor) c4(92349) c5(92350, 92351, 92352, 92353, 92354, 92355, 92356) c6( )c7(bB, bE, eM, X, gd); #c1(FDXR) c2(NP_001244942) c3(NADPH:adrenodoxin oxidoreductase, mitochondrial isoform 4) c4(92357) c5(92358, 92359, 92360, 92361, 92362, 92363) c6( ) c7(bB, bE, eM, X, gd); #c1(FDXR) c2(NP_001244943) c3(NADPH:adrenodoxin oxidoreductase, mitochondrial isoform 5 precursor) c4(92364) c5(92365, 92366, 92367, 92368, 92369, 92370) c6( )c7(bB, bE, eM, X, gd); #c1(FDXR) c2(NP_001244944) c3(NADPH:adrenodoxin oxidoreductase, mitochondrial isoform 6 precursor) c4(92371) c5(92372, 92373, 92374, 92375, 92376, 92377) c6( )c7(bB, bE, eM, X, gd); #c1(FDXR) c2(NP_001244945) c3(NADPH:adrenodoxin oxidoreductase, mitochondrial isoform 7) c4(92378) c5(92379, 92380, 92381, 92382, 92383, 92384) c6( )c7(bB, bE, eM, X, gd); #c1(FDXR) c2(NP_004101) c3(NADPH:adrenodoxin oxidoreductase, mitochondrial isoform 2 precursor) c4(143555) c5(143556, 143557, 143558, 143559, 143560, 143561, 143562, 143563, 143564, 143565) c6( )c7(bB, bE, eM, X, gd); #c1(FDXR) c2(XP_011522832) c3(NADPH:adrenodoxin oxidoreductase, mitochondrial isoform X5) c4( )c5(317328, 317329, 317330, 317331, 317332) c6( )c7(bB, bE, eM, X, gd); #c1(FDXR) c2(XP_011522835) c3(NADPH:adrenodoxin oxidoreductase, mitochondrial isoform X8) c4(317333) c5( )c6( )c7(bB, bE, eM, X, gd); #c1(FECH) c2(NP_000131) c3(ferrochelatase, mitochondrial isoform b precursor) c4(2880) c5(2881, 2882, 2883, 2884, 2885, 2886) c6( )c7(T, QW, bsl, bs, bB, n, ci, nE, q, br, eA, yw, nl, oW, aT, dV, dz, aMC, d, et, aW, hl, nh, KX, dq, CE, bw, bmj, dr, bsm, OC, aBr, iT, bE, QV); #c1(FECH) c2(NP_001012533) c3(ferrochelatase, mitochondrial isoform a precursor) c4(25087) c5(25088, 25089, 25090, 25091, 25092, 25093) c6( )c7(T, QW, bsl, bs, bB, n, ci, nE, q, br, eA, yw, nl, oW, aT, dV, dz, aMC, d, et, aW, hl, nh, KX, dq, CE, bw, bmj, dr, bsm, OC, aBr, iT, bE, QV); #c1(FEM1A) c2(NP_061178) c3(protein fem-1 homolog A) c4(199789) c5(199790, 199791, 199792, 199793, 199794, 199795, 199796) c6( )c7(aZ, kl, dD, eq, bv); #c1(FEM1B) c2(NP_056137) c3(protein fem-1 homolog B) c4(185895) c5(185896, 185897, 185898, 185899, 185900, 185901) c6( ) c7(bL, T, dD, kl, bE, aZ, bB, eq); #c1(FEM1C) c2(NP_064562) c3(protein fem-I homolog C) c4(202983) c5(202984, 202985, 202986, 202987, 202988, 202989) c6( )c7(aZ, kl, bv); #c1(FENI) c2(NP_004102) c3(flap endonuclease 1) c4(143566, 143567) c5(143568, 143569, 143570, 143571, 143572, 143573, 143574, 143575, 143576, 143577, 143578, 143579) c6( )c7(T, hy, j, V, q, eA, ad, av, aT, ae, B, BN, wr, aW, eZ, r, La, cl, bz, dR, bF, rR, gX, bD, ne); #c1(FER) c2(NP_005237) c3(tyrosine-protein kinase Fer) c4(154303, 154304) c5(154305, 154306, 154307, 154308, 154309, 154310, 154311) c6( )c7(adu, ae, T, x, bF, eM, fe, k, ei, bt, Bh, B, ad); #c1(FER) c2(XP_011541580) c3(tyrosine-protein kinase Fer isoform X8) c4(340ill) c5(340112, 340113, 340114, 340115, 340116, 340117) c6( )c7(adu, ae, T, x, bF, eM, fe, k, ei, bt, Bh, B, ad); #c1(FERD3L) c2(NP_690862) c3(fer3-like protein) c4(244991) c5(244992, 244993, 244994, 244995, 244996, 244997) c6( )c7(aw, fq, cd, ac); #c1(FERMT1) c2(NP_060141) c3(fermitin family homolog 1) c4(194392, 194393, 194394, 194395) c5(194396, 194397, 194398, 194399, 194400, 194401, 194402, 194403, 194404, 194405, 194406, 194407, 194408, 194409, 194410, 194411, 194412, 194413, 194414, 194415, 194416, 194417, 194418, 194419) c6( )c7(T, yk, bB, b, no, aE, nl, aT, dL, ji, K, lQ, bsn, eL, Us, li, aW, ff, nh, fe, ark, en, iV, arJ, bz, dr, bE, iu); #c1(FERMT2) c2(NP_001128471) c3(fermitin family homolog 2 isoform 2) c4(50012) c5(50013, 50014, 50015, 50016, 50017, 50018, 50019, 50020, 50021, 50022) c6( )c7(T, aA, bB, v, ad, hW, aT, By, dL, K, ae, aW, ark, kp, aF, arJ, op, bE, vA, dy, Hb, it); #c1(FERMT2) c2(NP_001128472) c3(fermitin family homolog 2 isoform 3) c4(50023) c5(50024, 50025, 50026, 50027, 50028, 50029, 50030, 50031, 50032, 50033, 50034, 50035) c6( )c7(T, aA, bB, v, ad, hW, aT, By, dL, K, ae, aW, ark, kp, aF, arJ, op, bE, vA, dy, Hb, it); #c1(FERMT2) c2(NP_006823) c3(fermitin family homolog 2 isoform 1) c4(168427, 168428) c5(168429, 168430, 168431, 168432, 168433, 168434, 168435, 168436, 168437, 168438) c6( )c7(T, aA, bB, v, ad, hW, aT, By, dL, K, ae, aW, ark, kp, aF, arJ, op, bE, vA, dy, Hb, it); #c1(FERMT3) c2(NP_113659) c3(fermitin family homolog 3 short form) c4(221982, 221983) c5(221984, 221985, 221986, 221987, 221988, 221989, 221990, 221991, 221992, 221993, 221994, 221995) c6( )c7(xu, hW, op, oV, zs, vA, bE, bfM, bB, ark, arJ, dr); #c1(FERMT3) c2(XP_011543596) c3(fermitin family homolog 3 isoform X1) c4(342615) c5(342616, 342617, 342618, 342619, 342620, 342621) c6( )c7(xu, hW, op, oV, zs, vA, bE, bfM, bB, ark, arJ, dr); #c1(FES) c2(NP_001137257) c3(tyrosine-protein kinase Fes/Fps isoform 4) c4(55173, 55174, 55175) c5(55176, 55177, 55178, 55179, 55180, 55181, 55182, 55183, 55184, 55185, 55186, 55187, 55188, 55189, 55190, 55191, 55192, 55193, 55194, 55195, 55196, 55197) c6( )c7(AX, ck, af, iT, Y, oe, iK, ew, r, cz, gl, v, ci, K, hf, bw, bt, bz, q); #c1(FES) c2(NP_001996) c3(tyrosine-protein kinase Fes/Fps isoform 1) c4(123009) c5(123010, 123011, 123012, 123013, 123014, 123015, 123016, 123017, 123018, 123019, 123020) c6( )c7(AX, ck, af, iT, Y, De, ik, ew, r, cz, gl, v, ci, K, hf, bw, bt, bz, q); #c1(FES) c2(XP_005254937) c3(tyrosine-protein kinase Fes/Fps isoform X1) c4(273380) c5(273381, 273382, 273383, 273384, 273385, 273386) c6( )c7(AX, ck, af, iT, Y, De, iK, ew, r, cz, gl, v, ci, K, hf, bw, bt, bz, q); #c1(FES) c2(XP_005254939) c3(tyrosine-protein kinase Fes/Fps isoform X2) c4(273387, 273388, 273389, 273390, 273391) c5(273392, 273393, 273394, 273395, 273396, 273397, 273398, 273399, 273400, 273401, 273402, 273403, 273404, 273405, 273406, 273407, 273408, 273409, 273410, 273411, 273412, 273413, 273414, 273415, 273416) c6( )c7(AX, ck, af, iT, Y, oe, ik, ew, r, cz, gl, v, ci, K, hf, bw, bt, bz, q); #c1(FES) c2(XP_011519662) c3(tyrosine-protein kinase Fes/Fps isoform X3) c4(314332) c5(314333, 314334, 314335, 314336, 314337, 314338) c6( )c7(AX, ck, af, iT, Y, oe, iK, ew, r, cz, gl, v, ci, K, hf, bw, bt, bz, q); #c1(FEV) c2(NP_059991) c3(protein FEV) c4(193538) c5(193539, 193540, 193541, 193542, 193543, 193544, 193545, 193546) c6( )c7(bM, am, aDy, df, bS, j, ct, pM, bv, bW, aT, cU, bl, K, Cy, dx, aa, ajV, Nr, sp, ap, Xm, EZ, Z, bz, DF, hq, Nq, TA, cT, aDu, cA, aCT, z, bjL, at, mf, aZo, afp); #c1(FEZ1) c2(NP_005094) c3(fasciculation and elongation protein zeta-1 isoform 1) c4(152844, 152845) c5(152846, 152847, 152848, 152849, 152850, 152851) c6( )c7(ck, T, id, aW, eL, AQ, ff, aT, gl, dL, bK, ci, en, Tp, aF, he, bz, ad); #c1(FEZ1) c2(NP_072043) c3(fasciculation and elongation protein zeta-1 isoform 2) c4( )c5(212429, 212430, 212431, 212432, 212433, 212434, 212435, 212436, 212437, 212438) c6( )c7(ck, T, id, aW, eL, AQ, ff, aT, gl, dL, bK, ci, en, Tp, aF, he, bz, ad); #c1(FEZ2) c2(NP_001036013) c3(fasciculation and elongation protein zeta-2 isoform 2) c4(33700) c5(33701, 33702, 33703, 33704, 33705, 33706) c6( )c7(j); #c1(FEZ2) c2(NP_005093) c3(fasciculation and elongation protein zeta-2 isoform 1) c4(152830, 152831) c5(152832, 152833, 152834, 152835, 152836, 152837, 152838, 152839, 152840, 152841, 152842, 152843) c6( )c7(j); #c1(FEZF1) c2(NP_001019784) c3(fez family zinc finger protein 1 isoform 1) c4(27826, 27827, 27828) c5(27829, 27830, 27831, 27832, 27833, 27834, 27835, 27836, 27837, 27838, 27839, 27840) c6( )c7(T, aF, yn, bN, aA); #c1(FEZF1) c2(NP_001153736) c3(fez family zinc finger protein 1 isoform 2) c4(60951) c5(60952, 60953, 60954, 60955, 60956, 60957, 60958, 60959, 60960, 60961, 60962, 60963, 60964) c6( )c7(T, aF, yn, bN, aA); #c1(FEZF2) c2(NP_060478) c3(fez family zinc finger protein 2) c4(196140) c5(196141, 196142, 196143, 196144, 196145, 196146, 196147, 196148, 196149, 196150, 196151) c6( ) c7(QK); #c1(FEZF2) c2(XP_011532183) c3(fez family zinc finger protein 2 isoform X1) c4( )c5(328228, 328229, 328230, 328231) c6( ) c7(QK); #c1(FFAR1) c2(NP_005294) c3(free fatty acid receptor 1) c4(154740) c5(154741, 154742, 154743, 154744, 154745, 154746) c6( ) c7(bL, T, mj, aW, AR, fc, aT, blg, mf, aj, v, cP, cm, J); #c1(FFAR2) c2(NP_005297) c3(free fatty acid receptor 2) c4(154757) c5(154758, 154759, 154760, 154761, 154762, 154763) c6( )c7(oO, dq, T, e, v); #c1(FFAR3) c2(NP_005295) c3(free fatty acid receptor 3) c4( ) c5(154747, 154748, 154749, 154750, 154751, 154752, 154753, 154754, 154755, 154756) c6( )c7(v); #c1(FFAR3) c2(XP_011525160) c3(free fatty acid receptor 3 isoform X1) c4(319608, 319609) c5(319610, 319611) c6( )c7(v); #c1(FFAR4) c2(NP_001182684) c3(free fatty acid receptor 4 isoform GPR120-S) c4(75147) c5(75148, 75149, 75150, 75151, 75152, 75153) c6( )c7(bL, T, cF, fc, aj, bso, v, q, J); #c1(FFAR4) c2(NP_859529) c3(free fatty acid receptor 4 isoform GPR120-L) c4(255543) c5(255544, 255545, 255546, 255547, 255548, 255549) c6( )c7(bL, T, cF, fc, aj, bso, v, q, J); #c1(FGA) c2(NP_000499) c3(fibrinogen alpha chain isoform alpha-E preproprotein) c4(10307) c5(10308, 10309, 10310, 10311, 10312, 10313, 10314, 10315, 10316, 10317, 10318, 10319, 10320, 10321, 10322, 10323, 10324, 10325, 10326, 10327, 10328, 10329, 10330, 10331, 10332, 10333, 10334, 10335, 10336, 10337, 10338, 10339, 10340, 10341, 10342, 10343, 10344, 10345, 10346, 10347, 10348, 10349, 10350, 10351, 10352, 10353, 10354, 10355, 10356, 10357, 10358, 10359, 10360, 10361, 10362, 10363, 10364, 10365, 10366, 10367, 10368, 10369, 10370, 10371, 10372, 10373, 10374, 10375, 10376, 10377, 10378, 10379, 10380, 10381, 10382, 10383, 10384, 10385, 10386, 10387, 10388, 10389, 10390, 10391, 10392, 10393, 10394, 10395, 10396, 10397, 10398, 10399, 10400, 10401, 10402, 10403, 10404, 10405, 10406, 10407, 10408, 10409, 10410, 10411, 10412, 10413, 10414, 10415, 10416, 10417, 10418, 10419, 10420, 10421, 10422, 10423, 10424, 10425, 10426, 10427, 10428, 10429, 10430, 10431, 10432, 10433, 10434, 10435, 10436, 10437, 10438, 10439, 10440, 10441, 10442, 10443, 10444, 10445, 10446, 10447, 10448, 10449, 10450, 10451, 10452) c6(10453) c7(P, bL, dD, ahf, aXW, qb, fV, ea, e, aw, cz, vr, v, j, ah, C, eq, av, dY, jk, bsp, M, cy, bq, bsq, amO, af, bA, bpU, eu, bsr, hJ, cm, ek, aUF, ar, W, ac, ag, E, Km, aS, R, dy, lC, aLL); #c1(FGA) c2(NP_068657) c3(fibrinogen alpha chain isoform alpha preproprotein) c4(209934, 209935, 209936, 209937, 209938) c5(209939, 209940, 209941, 209942, 209943, 209944, 209945, 209946, 209947, 209948, 209949, 209950, 209951) c6( )c7(P, bL, dD, ahf, aXW, qb, fV, ea, e, aw, cz, vr, v, j, ah, C, eq, av, dY, jk, bsp, M, cy, bq, bsq, amO, af, bA, bpU, eu, bsr, hJ, cm, ek, aUF, ar, W, ac, ag, E, Km, aS, R, dy, lC, aLL); #c1(FGB) c2(NP_005132) c3(fibrinogen beta chain isoform 1 preproprotein) c4(153127, 153128) c5(153129, 153130, 153131, 153132, 153133, 153134, 153135, 153136, 153137, 153138, 153139, 153140, 153141, 153142, 153143, 153144, 153145, 153146, 153147, 153148, 153149, 153150, 153151, 153152, 153153, 153154, 153155, 153156, 153157, 153158, 153159, 153160, 153161, 153162, 153163, 153164, 153165, 153166, 153167, 153168, 153169, 153170, 153171, 153172, 153173, 153174, 153175, 153176, 153177, 153178, 153179, 153180, 153181, 153182, 153183, 153184, 153185, 153186, 153187, 153188, 153189, 153190, 153191, 153192, 153193, 153194, 153195, 153196, 153197, 153198, 153199, 153200, 153201, 153202, 153203, 153204, 153205, 153206, 153207, 153208, 153209, 153210, 153211, 153212, 153213, 153214, 153215, 153216, 153217, 153218, 153219, 153220, 153221, 153222, 153223, 153224, 153225, 153226, 153227, 153228, 153229, 153230, 153231, 153232, 153233, 153234, 153235, 153236, 153237, 153238, 153239, 153240, 153241, 153242, 153243, 153244, 153245, 153246, 153247, 153248, 153249, 153250, 153251, 153252, 153253, 153254, 153255, 153256, 153257, 153258, 153259, 153260, 153261, 153262, 153263, 153264, 153265, 153266, 153267, 153268, 153269, 153270, 153271, 153272, 153273, 153274, 153275, 153276, 153277, 153278) c6( )c7(bL, sY, dD, aSz, aFe, eR, qb, ea, wL, oe, e, aw, cz, cR, OR, vr, v, j, eq, C, aY, mC, Tz, TB, dY, vh, vC, rS, jk, M, cy, an, af, eu, hm, sj, bA, eZ, fe, cM, aVc, uC, pb, eK, ar, kT, sn, c, W, ac, ag, vb, aFb, lf, sG, at, sU, te, tz, aS, fl, dy, uz, pw, lC, ei, eo, wx, y, aLL); #c1(FGD1) c2(NP_004454) c3(FYVE, RhoGEF and PH domain-containing protein 1) c4(146446) c5(146447, 146448, 146449, 146450, 146451, 146452, 146453) c6( )c7(ak, xb, aW, eF, eH, Az, afU, aT, nf, K, hK, aOV, dr); #c1 (FGD2) c2(NP_775829) c3(FYVE, RhoGEF and PH domain-containing protein 2) c4(250460, 250461) c5(250462, 250463, 250464, 250465, 250466, 250467, 250468) c6( )c7(fe); #c1(FGD2) c2(XP_011512676) c3(FYVE, RhoGEF and PH domain-containing protein 2 isoform X7) c4( )c5(303688, 303689, 303690, 303691, 303692) c6( )c7(fe); #c1(FGD3) c2(NP_001077005) c3(FYVE, RhoGEF and PH domain-containing protein 3 isoform a) c4(37587, 37588) c5(37589, 37590, 37591, 37592, 37593, 37594, 37595, 37596, 37597, 37598, 37599, 37600, 37601, 37602, 37603, 37604) c6( )c7(hd, afU); #c1(FGD3) c2(NP 001273922) c3(FYVE, RhoGEF and PH domain-containing protein 3 isoform b) c4(105896) c5(105897, 105898, 105899, 105900, 105901, 105902) c6( )c7(hd, afU); #c1(FGD4) c2(NP_001291409) c3(FYVE, RhoGEF and PH domain-containing protein 4 isoform 1) c4(115936, 115937) c5(115938, 115939, 115940, 115941, 115942, 115943, 115944, 115945, 115946, 115947, 115948, 115949, 115950) c6( )c7(ac, Rz, Pt, agU, fu, bU, fq, bss, sj); #c1(FGD4) c2(NP_001291410) c3(FYVE, RhoGEF and PH domain-containing protein 4 isoform 2) c4(115951) c5(115952, 115953, 115954, 115955, 115956, 115957) c6( )c7(ac, Rz, Pt, agU, fu, bU, fq, bss, sj); #c1(FGD4) c2(NP_001291412) c3(FYVE, RhoGEF and PH domain-containing protein 4 isoform 4) c4(115964) c5(115965, 115966, 115967, 115968, 115969, 115970, 115971, 115972, 115973, 115974, 115975) c6( )c7(ac, Rz, Pt, agU, fu, bu, fq, bss, sj); #c1(FGD4) c2(XP_005253361) c3(FYVE, RhoGEF and PH domain-containing protein 4 isoform X2) c4(272442) c5(272443, 272444, 272445, 272446, 272447, 272448) c6( )c7(ac, Rz, Pt, agU, fu, bU, fq, bss, sj); #c1(FGD4) c2(XP_011518856) c3(FYVE, RhoGEF and PH domain-containing protein 4 isoform X3) c4(313204) c5( )c6( )c7(ac, Rz, Pt, agU, fu, bU, fq, bss, sj); #c1(FGD4) c2(XP_011518858) c3(FYVE, RhoGEF and PH domain-containing protein 4 isoform X1) c4(313205, 313206) c5(313207, 313208, 313209, 313210, 313211, 313212, 313213, 313214, 313215, 313216, 313217) c6( )c7(ac, Rz, Pt, agU, fu, bU, fq, bss, sj); #c1(FGD4) c2(XP_011518860) c3(FYVE, RhoGEF and PH domain-containing protein 4 isoform X6) c4( )c5(313218) c6( )c7(ac, Rz, Pt, agU, fu, bU, fq, bss, sj); #c1(FGD5) c2(NP_689749) c3(FYVE, RhoGEF and PH domain-containing protein 5) c4(243841) c5(243842, 243843, 243844, 243845, 243846, 243847) c6( )c7(aT); #c1(FGD5) c2(XP_011531724) c3(FYVE, RhoGEF and PH domain-containing protein 5 isoform X1) c4( )c5(327815) c6( )c7(aT); #c1(FGD5) c2(XP_011531726) c3(FYVE, RhoGEF and PH domain-containing protein 5 isoform X3) c4(327816) c5(327817, 327818, 327819, 327820, 327821, 327822, 327823) c6( )c7(aT); #c1(FGD6) c2(NP_060821) c3(FYVE, RhoGEF and PH domain-containing protein 6) c4(197986, 197987, 197988) c5(197989, 197990, 197991, 197992, 197993, 197994, 197995, 197996, 197997, 197998, 197999, 198000, 198001, 198002, 198003, 198004, 198005, 198006) c6( )c7(j); #c1(FGF1) c2(NP_001244139) c3(fibroblast growth factor 1 isoform 1 precursor) c4(91980, 91981, 91982) c5(91983, 91984, 91985, 91986, 91987, 91988, 91989, 91990, 91991, 91992, 91993, 91994, 91995, 91996, 91997, 91998, 91999, 92000, 92001, 92002, 92003, 92004, 92005, 92006, 92007, 92008, 92009, 92010, 92011, 92012, 92013, 92014, 92015, 92016, 92017, 92018, 92019) c6( )c7(sY, T, dD, Js, pf, ea, fG, qx, fg, cz, fd, v, j, ci, eq, sl, Vh, eA, ad, eJ, wD, dP, av, Na, ft, F, ib, Rq, rw, aT, FN, lG, cy, us, dL, K, Dz, Nc, ae, af, eu, WZ, eL, LC, Y, aW, vj, eH, hp, g, eF, t, uC, bw, op, ck, il, ac, bP, U, gZ, m, y, bt, Jo); #c1(FGF1) c2(NP_149127) c3(fibroblast growth factor 1 isoform 2 precursor) c4(227342) c5(227343, 227344, 227345, 227346, 227347) c6( )c7(sY, T, dD, Js, pf, ea, fG, qx, fg, cz, fd, v, j, ci, eq, sl, Vh, eA, ad, eJ, wD, dP, av, Na, ft, F, ib, Rq, rw, aT, FN, lG, cy, us, dL, K, Dz, Nc, ae, af, eu, WZ, eL, LC, Y, aW, vj, eH, hp, g, eF, t, uC, bw, op, ck, il, ac, bP, U, gZ, m, y, bt, Jo); #c1(FGF1) c2(XP_005268447) c3(fibroblast growth factor 1 isoform X2) c4(281812) c5(281813, 281814, 281815, 281816, 281817, 281818, 281819, 281820, 281821) c6( )c7(sY, T, dD, Js, pf, ea, fG, qx, fg, cz, fd, v, j, ci, eq, sl, Vh, eA, ad, eJ, wD, dP, av, Na, ft, F, ib, Rq, rw, aT, FN, IG, cy, us, dL, K, Dz, Nc, ae, af, eu, WZ, eL, LC, Y, aW, vj, eH, hp, g, ef, t, uC, bw, op, ck, il, ac, bP, U, gZ, m, y, bt, Jo); #c1(FGF1) c2(XP_011535910) c3(fibroblast growth factor 1 isoform X1) c4(333/70) c5(333171, 333172, 333173, 333174, 333175, 333176) c6( )c7(sY, T, dD, Js, pf, ea, fG, qx, fg, cz, fd, v, j, ci, eq, sl, Vh, eA, ad, eJ, wD, dP, av, Na, ft, F, ib, Rq, rw, aT, FN, lG, cy, us, dL, K, Dz, Nc, ae, af, eu, WZ, eL, LC, Y, aW, vj, eH, hp, g, eF, t, uC, bw, op, ck, il, ac, bP, U, gZ, m, y, bt, Jo); #c1(FGF10) c2(XP_005248321) c3(fibroblast growth factor 10 isoform X1) c4(268303, 268304) c5(268305, 268306, 268307, 268308, 268309, 268310, 268311, 268312, 268313, 268314, 268315, 268316, 268317) c6( )c7(T, aC, bsy, bsv, bsu, bsx, ci, adg, zP, ad, bsz, Na, aE, lg, aT, Nc, bsA, aW, vj, yL, Pp, cl, bz, bst, ll, Be, bhl, bD, bsw, bmY); #c1(FGF11) c2(NP_004103) c3(fibroblast growth factor 1l isoform 1) c4(143580) c5(143581, 143582, 143583, 143584, 143585, 143586, 143587) c6( )c7(ajM); #c1(FGF12) c2(NP_004104) c3(fibroblast growth factor (2 isoform 2) c4(143588, 143589) c5(143590, 143591, 143592, 143593, 143594, 143595, 143596, 143597, 143598, 143599, 143600, 143601, 143602, 143603, 143604, 143605, 143606, 143607, 143608, 143609, 143610, 143611, 143612, 143613, 143614, 143615, 143616) c6( )c7(ajM, ff, an, en, aOX); #c1(FGF12) c2(NP_066360) c3(fibroblast growth factor 12 isoform 1) c4(207195, 207196) c5(207197, 207198, 207199, 207200, 207201, 207202, 207203) c6( ) c7(ajM, ff, an, en, aOX); #c1(FGF13) c2(NP_001132970) c3(fibroblast growth factor 13 isoform 4) c4(52734) c5(52735, 52736, 52737, 52738, 52739, 52740, 52741) c6( )c7(ID, bM, T, aC, pf, ea, if, bO, ci, Vp, aE, K, hu, eL, li, eG, LC, aW, g, eF, cm, zV, iP, eM, nB, AM, vA, nw, m, bsB, ik, dD, ht, n, aj, ct, nA, qN, eW, amB, ft, Nn, h, eV, aq, op, W, Be, hR, B, AB, fM, wj, ex, aA, cR, v, amC, q, J, eA, mj, aT, cy, jj, bau, ae, af, s, il, Y, r, lo, t, ik, Vn, rR, kP, dy, auN, bt, yv, jh, bS, eq, ad, BY, dP, hd, ib, WZ, d, lR, eH, nf, aF, Z, bsC, MJ, lC, y); #c1(FGF13) c2(NP_001132974) c3(fibroblast growth factor 13 isoform 3) c4( )c5(52742, 52743, 52744, 52745, 52746, 52747, 52748, 52749, 52750, 52751) c6( )c7(ID, bM, T, aC, pf, ea, if, bO, ci, Vp, aE, K, hu, eL, li, eG, LC, aW, g, eF, cm, zV, iP, eM, nB, AM, vA, nw, m, bsB, ik, dD, ht, n, aj, ct, nA, qN, eW, amB, ft, Nn, h, eV, aq, op, W, Be, hR, B, AB, fM, wj, ex, aA, cR, v, amC, q, J, eA, mj, aT, cy, jj, bau, ae, af, s, iU, Y, r, lo, t, ik, Vn, rR, kP, dy, auN, bt, yv, jh, bS, eq, ad, BY, dP, hd, ib, WZ, d, lR, eH, nf, aF, Z, bsC, MJ, lC, y); #c1(FGF13) c2(NP_004105) c3(fibroblast growth factor 13 isoform 1) c4(143617) c5(143618, 143619, 143620, 143621, 143622, 143623, 143624) c6( )c7(ID, bM, T, aC, pf, ea, if, bO, ci, Vp, aE, K, hu, eL, li, eG, LC, aW, g, eF, cm, zV, iP, eM, nB, AM, vA, nw, m, bsB, ik, dD, ht, n, aj, ct, nA, qN, eW, amB, ft, Nn, h, eV, aq, op, W, Be, hR, B, AB, fM, wj, ex, aA, cR, v, amC, q, J, eA, mj, aT, cy, jj, bau, ae, af, s, il, Y, r, lo, t, ik, Vn, rR, kP, dy, auN, bt, yv, jh, bS, eq, ad, BY, dP, hd, ib, WZ, d, lR, eH, nf, aF, Z, bsC, MJ, lC, y); #c1(FGF13) c2(NP_378668) c3(fibroblast growth factor 13 isoform 5) c4(228941) c5(228942, 228943, 228944, 228945, 228946, 228947, 228948) c6( )c7(ID, bM, T, aC, pf, ea, if, bO, ci, Vp, aE, K, hu, eL, li, eG, LC, aW, g, eF, cm, zV, iP, eM, nB, AM, vA, nw, m, bsB, ik, dD, ht, n, aj, ct, nA, qN, eW, amB, ft, Nn, h, eV, aq, op, W, Be, hR, B, AB, fM, wj, ex, aA, cR, v, amC, q, J, eA, mj, aT, cy, jj, bau, ae, af, s, il, Y, r, lo, t, ik, Vn, rR, kP, dy, auN, bt, yv, jh, bS, eq, ad, BY, dP, hd, ib, WZ, d, lR, eH, nf, aF, Z, bsC, MJ, lC, y); #c1(FGF13) c2(XP_005262456) c3(fibroblast growth factor 13 isoform X1) c4(277930, 277931, 277932, 277933, 277934, 277935) c5(277936, 277937, 277938, 277939, 277940, 277941, 277942, 277943, 277944, 277945, 277946, 277947, 277948, 277949, 277950, 277951, 277952, 277953, 277954, 277955, 277956, 277957, 277958, 277959, 277960, 277961, 277962) c6( )c7(ID, bM, T, aC, pf, ea, if, bO, ci, Vp, aE, K, hu, eL, li, eG, LC, aW, g, eF, cm, zV, iP, eM, nB, AM, vA, nw, m, bsB, ik, dD, ht, n, aj, ct, nA, qN, eW, amB, ft, Nn, h, eV, aq, op, W, Be, hR, B, AB, fM, wj, ex, aA, cR, v, amC, q, J, eA, mj, aT, cy, jj, bau, ae, af, s, il, Y, r, lo, t, ik, Vn, rR, kP, dy, auN, bt, yv, jh, bS, eq, ad, BY, dP, hd, ib, WZ, d, lR, eH, nf, aF, Z, bsC, MJ, lC, y); #c1(FGF13) c2(XP_011529617) c3(fibroblast growth factor 13 isoform X2) c4( )c5(325143, 325144) c6( )c7(ID, bM, T, aC, pf, ea, if, bO, ci, Vp, aE, K, hu, eL, li, eG, LC, aW, g, eF, cm, zV, iP, eM, nB, AM, vA, nw, m, bsB, ik, dD, ht, n, aj, ct, nA, qN, eW, amB, ft, Nn, h, eV, aq, op, W, Be, hR, B, AB, fM, wj, ex, aA, cR, v, amC, q, J, eA, mj, aT, cy, jj, bau, ae, af, s, il, Y, r, lo, t, ik, Vn, rR, kP, dy, auN, bt, yv, jh, bS, eq, ad, BY, dP, hd, ib, WZ, d, lR, eH, nf, aF, Z, bsC, MJ, lC, y); #c1(FGF14) c2(NP_004106) c3(fibroblast growth factor 14 isoform (A) c4(143625) c5(143626, 143627, 143628, 143629, 143630, 143631, 143632) c6( )c7(ky, kB, hb, bsD, rf, yZ, bsE, ajM, Ea, xg, cd, aRo); #c1(FGF14) c2(NP_787125) c3(fibroblast growth factor 14 isoform (B) c4(252135) c5(252136, 252137, 252138, 252139, 252140, 252141, 252142) c6( )c7(ky, kB, hb, bsD, rf, yZ, bsE, ajM, Ea, xg, cd, aRo); #c1(FGF16) c2(NP_003859) c3(fibroblast growth factor 16) c4(141565, 141566) c5(141567, 141568, 141569, 141570, 141571, 141572, 141573) c6( )c7(T, yt, qK, bsF, m, t); #c1(FGF17) c2(NP_001291407) c3(fibroblast growth factor 17 isoform 2 precursor) c4(115924) c5(115925, 115926, 115927, 115928, 115929, 115930, 115931, 115932) c6(115933, 115934) c7(G, bsG, aW, aT, Xr, ae, B, ad); #c1(FGF17) c2(NP_003858) c3(fibroblast growth factor 17 isoform 1 precursor) c4(141553) c5(141554, 141555, 141556, 141557, 141558, 141559, 141560, 141561, 141562) c6(141563, 141564) c7(G, bsG, aW, aT, Xr, ae, B, ad); #c1(FGF18) c2(NP_003853) c3(fibroblast growth factor 18 precursor) c4(141427, 141428) c5(141429, 141430, 141431, 141432, 141433, 141434, 141435, 141436, 141437, 141438, 141439, 141440, 141441, 141442, 141443, 141444, 141445, 141446, 141447, 141448, 141449, 141450, 141451, 141452, 141453, 141454, 141455, 141456, 141457, 141458, 141459, 141460, 141461, 141462, 141463, 141464, 141465, 141466, 141467, 141468, 141469, 141470, 141471, 141472, 141473, 141474, 141475, 141476, 141477, 141478, 141479, 141480, 141481, 141482, 141483, 141484, 141485, 141486, 141487, 141488, 141489, 141490, 141491, 141492, 141493, 141494, 141495, 141496, 141497, 141498, 141499, 141500, 141501, 141502, 141503, 141504, 141505, 141506, 141507, 141508, 141509, 141510, 141511, 141512, 141513, 141514, 141515, 141516, 141517, 141518, 141519, 141520, 141521, 141522) c6(141523) c7(kl, B, bE, Y, De, X, wk, U, r, bB, Nc, nw, iJ, K, nC, fq, q); #c1(FGF19) c2(NP_005108) c3(fibroblast growth factor 19 precursor) c4(152956) c5(152957, 152958, 152959, 152960, 152961, 152962, 152963) c6( )c7(T, aZ, al, bB, aj, v, J, ad, bsH, h, ji, ae, kl, B, ev, dq, aq, kp, iR, fq, GR, jq, gk, bE, mg, it, X); #c1(FGF2) c2(NP_001997) c3(fibroblast growth factor 2) c4(123021, 123022) c5(123023, 123024, 123025, 123026, 123027, 123028, 123029) c6( )c7(oO, bM, G, T, lD, aC, pf, ea, if, al, bO, fd, bB, qj, ci, lR, DB, pC, amW, aDI, ip, x, Vp, Na, aE, rw, Qk, iv, K, hu, DY, eL, cp, li, eG, yo, JI, LC, aW, LZ, g, as, eF, ir, uC, cm, zV, iP, yX, qJ, y, eM, nB, AM, iA, vA, vy, cj, sv, nw, m, bsB, fy, ik, iu, de, FW, dD, lT, aw, ht, qy, n, aj, fr, ct, uE, qN, cd, asS, El, ai, eW, amB, ft, amJ, kl, Nn, Cf, h, lG, eV, dL, vx, bsl, dx, Dg, bsK, A, fQ, aq, fe, yq, cM, op, W, bP, dB, eD, wk, bsO, mg, Be, hL, hR, at, pg, it, B, bL, AB, fM, dA, wj, fG, nx, eX, bsN, aA, CR, cz, hy, v, j, amC, q, J, eA, xx, aHu, qK, HC, aT, cy, dz, rO, rD, Nc, bq, bau, ae, af, dl, bA, jf, s, iU, xR, Y, bsl, r, cu, lo, en, hf, cl, t, ik, bQ, pb, Vn, ck, sS, rR, kP, c, Dy, wg, wD, nM, il, dy, oX, auN, bt, Mj, yv, hS, sY, kh, id, acv, pY, aD, CI, iW, jh, bS, dG, ah, eq, bsM, Fc, ad, wD, BY, Jm, dP, hd, F, ib, oW, Dj, aNR, uD, adx, nJ, WZ, DI, d, BN, lR, lU, cW, ff, eH, aF, iV, Z, jb, bz, wW, eK, LB, es, D, pu, pc, Uj, bE, MJ, aS, GF, lC, ne, us, bsJ); #c1(FGF20) c2(NP_062825) c3(fibroblast growth factor 20) c4(202160, 202161) c5(202162, 202163, 202164, 202165, 202166, 202167, 202168, 202169, 202170, 202171) c6( )c7(bsP, T, s, fe, an, GB); #c1(FGF21) c2(XP_005258788) c3(fibroblast growth factor 21 isoform X1) c4(275734) c5(275735, 275736, 275737, 275738, 275739, 275740, 275741, 275742, 275743, 275744, 275745, 275746, 275747, 275748, 275749, 275750, 275751, 275752, 275753, 275754, 275755, 275756, 275757, 275758, 275759, 275760, 275761, 275762, 275763, 275764, 275765, 275766, 275767, 275768, 275769, 275770, 275771, 275772, 275773, 275774, 275775, 275776, 275777, 275778, 275779, 275780, 275781, 275782, 275783, 275784, 275785, 275786, 275787, 275788, 275789, 275790, 275791, 275792, 275793, 275794, 275795, 275796, 275797, 275798, 275799, 275800, 275801, 275802, 275803, 275804, 275805, 275806, 275807, 275808, 275809, 275810, 275811, 275812, 275813, 275814, 275815, 275816, 275817, 275818, 275819, 275820, 275821, 275822, 275823, 275824, 275825, 275826, 275827, 275828, 275829, 275830, 275831, 275832, 275833, 275834) c6(275835) c7(bL, T, e, cz, aj, v, j, J, KB, hb, iX, oj, Ak, h, cy, jy, K, bq, dl, bsQ, A, fc, dq, aq, kC, GR, KB, awN, mg, cP, ei); #c1(FGF23) c2(NP_065689) c3(fibroblast growth factor 23 precursor) c4(204836, 204837, 204838, 204839, 204840, 204841, 204842) c5(204843, 204844, 204845, 204846, 204847, 204848, 204849, 204850, 204851, 204852, 204853, 204854, 204855, 204856, 204857, 204858, 204859, 204860, 204861, 204862, 204863, 204864, 204865, 204866, 204867, 204868, 204869, 204870, 204871, 204872, 204873, 204874, 204875, 204876, 204877, 204878, 204879, 204880, 204881, 204882, 204883, 204884, 204885, 204886, 204887, 204888, 204889, 204890, 204891, 204892, 204893, 204894, 204895, 204896, 204897, 204898, 204899, 204900, 204901, 204902, 204903, 204904, 204905, 204906, 204907, 204908, 204909, 204910, 204911, 204912, 204913, 204914, 204915, 204916, 204917, 204918, 204919, 204920, 204921, 204922, 204923, 204924, 204925, 204926, 204927, 204928, 204929, 204930, 204931, 204932, 204933, 204934, 204935, 204936, 204937) c6(204938) c7(T, AB, eR, e, Ay, aj, amC, j, bqV, aY, pd, J, ad, adf, fU, bsU, amB, zw, MR, yw, oW, bsW, hP, bsR, lQ, bta, zu, bsX, ae, af, azh, aly, bmg, OX, vj, Hz, ev, arp, zq, bsZ, akb, bfH, AA, HI, dr, alx, amK, arr, acW, Of, bsY, eD, fP, gZ, lO, Km, bsS, bsT, aS, dy, bfl, bsV, azg, lS, ars, azr); #c1(FGF3) c2(NP_005238) c3(fibroblast growth factor 3 precursor) c4(154312) c5(154313, 154314, 154315, 154316, 154317, 154318, 154319) c6( ) c7(btc, T, fM, dA, pY, fZ, aA, n, Hn, aml, ci, oc, q, YD, er, Na, lg, aMT, aT, h, Nc, dL, hu, avt, fb, Cy, eL, Yi, ia, OV, alX, vT, aW, Y, ff, Az, aF, yq, en, hf, alZ, t, cl, bz, btb, zV, g, ik, ck, kn, bw, bqH, bE, AM, mG, vy, hL, btd, m, bt, it, B); #c1(FGF4) c2(XP_006718538) c3(fibroblast growth factor 4 isoform X1) c4(291894) c5(291895, 291896, 291897, 291898, 291899, 291900, 291901, 291902) c6(291903, 291904) c7(sY, T, lT, wu, ht, aA, Hn, n, fd, j, ci, q, aT, cy, dL, K, wm, hu, nJ, eL, yo, A, s, vT, aW, Y, ff, r, af, en, t, uC, zV, ck, eD, cj, m, bt, it, B); #c1(FGF5) c2(NP_004455) c3(fibroblast growth factor 5 isoform 1 precursor) c4(146454) c5(146455, 146456, 146457, 146458, 146459, 146460, 146461, 146462, 146463) c6( )c7(T, d, BN, il, dB, Y, U, cz, hy, nw, fr, g, nC, ne); #c1(FGF5) c2(NP_149134) c3(fibroblast growth factor 5 isoform 2 precursor) c4(227401) c5(227402, 227403, 227404, 227405, 227406, 227407) c6( )c7(T, d, BN, il, dB, Y, U, cz, hy, nw, fr, g, nC, ne); #c1(FGF6) c2(NP_066276) c3(fibroblast growth factor 6 precursor) c4(206923) c5(206924, 206925, 206926, 206927, 206928, 206929, 206930) c6( )c7(ck, YL, T, jf, r, aA, PJ, cm, ae, q, ad); #c1(FGF7) c2(NP_002000) c3(fibroblast growth factor 7 precursor) c4(123030) c5(123031, 123032, 123033, 123034, 123035, 123036, 123037, 123038, 123039, 123040, 123041, 123042, 123043, 123044, 123045, 123046, 123047, 123048, 123049, 123050, 123051, 123052, 123053, 123054, 123055, 123056, 123057, 123058, 123059, 123060, 123061, 123062, 123063, 123064, 123065, 123066, 123067, 123068, 123069, 123070, 123071, 123072, 123073, 123074, 123075, 123076, 123077, 123078, 123079, 123080, 123081, 123082, 123083, 123084, 123085, 123086, 123087, 123088, 123089, 123090, 123091, 123092, 123093, 123094, 123095, 123096, 123097, 123098, 123099, 123100, 123101, 123102, 123103, 123104, 123105, 123106, 123107, 123108, 123109, 123110, 123111, 123112, 123113, 123114, 123115, 123116, 123117, 123118, 123119, 123120, 123121, 123122, 123123, 123124, 123125, 123126, 123127, 123128, 123129, 123130, 123131, 123132, 123133, 123134) c6(123135, 123136, 123137, 123138, 123139) c7(G, T, aC, Ht, fG, Lx, bte, ht, aA, fF, al, Hn, ci, qN, sD, q, ad, asS, eA, Na, Vp, amB, aHu, aE, qg, arV, aT, lG, Nc, ji, K, lQ, aDZ, bsA, MO, nJ, ae, ao, JI, aW, ff, bg, cr, r, fe, Um, kp, aF, en, cl, g, t, iV, LB, bE, AM, wk, uR, wB, hl, at, uJ, m, fD, azr, jr, btf); #c1(FGF8) c2(NP_006110) c3(fibroblast growth factor 8 isoform B precursor) c4(161939, 161940) c5(161941, 161942, 161943, 161944, 161945, 161946, 161947) c6( )c7(gh, T, jW, Dh, RX, Hn, ci, bth, agY, iJ, ad, yn, Mg, Tg, anB, Na, bti, gH, ath, aT, Nc, nJ, ae, avP, btg, aW, fO, Az, btj, Pp, si, t, cm, amf, eD, RZ, m, UF, ZW, B, LS); #c1(FGF8) c2(NP_149353) c3 (fibroblast growth factor 8 isoform F precursor) c4(227475) c5(227476, 227477, 227478, 227479, 227480, 227481, 227482) c6( )c7(gh, T, jW, Dh, RX, Hn, ci, bth, agY, iJ, ad, yn, Mg, Tg, anB, Na, bti, gH, ath, aT, Nc, nJ, ae, avP, btg, aW, fO, Az, btj, Pp, si, t, cm, amF, eD, RZ, m, UF, ZW, B, LS); #c1(FGF8) c2(NP_149354) c3(fibroblast growth factor 8 isoform E precursor) c4(227483) c5(227484, 227485, 227486, 227487, 227488, 227489, 227490) c6( )c7(gh, T, jW, Dh, RX, Hn, ci, bth, agY, iJ, ad, yn, Mg, Tg, anB, Na, bti, gH, ath, aT, Nc, nJ, ae, avP, btg, aW, fO, Az, btj, Pp, si, t, cm, amF, eD, RZ, m, UF, ZW, B, LS); #c1(FGF8) c2(NP_149355) c3(fibroblast growth factor 8 isoform A precursor) c4(227491) c5(227492, 227493, 227494, 227495, 227496, 227497, 227498, 227499) c6( )c7(gh, T, jW, Dh, RX, Hn, ci, bth, agY, iJ, ad, yn, Mg, Tg, anB, Na, bti, gH, ath, aT, Nc, nJ, ae, avP, btg, aW, fO, Az, btj, Pp, si, t, cm, amF, eD, RZ, m, UF, ZW, B, LS); #c1(FGF9) c2(NP_002001) c3(fibroblast growth factor 9 precursor) c4(123140) c5(123141, 123142, 123143, 123144, 123145, 123146, 123147, 123148) c6( )c7(T, akc, zX, EQ, aA, bB, hy, Ul, q, ad, eA, ip, dP, xx, av, iu, Na, dY, btk, vJ, btl, UD, Nc, ae, ant, d, BN, vj, ff, la, gl, akY, aF, en, cl, g, zV, wW, eM, dB, bE, eD, aND, ne); #c1(FGFBP1) c2(NP_005121) c3(fibroblast growth factor-binding protein 1 precursor) c4(153053) c5(153054, 153055, 153056, 153057, 153058, 153059, 153060, 153061, 153062, 153063) c6( )c7(ar, cF, eJ, bE, aW, ff, r, aT, cz, h, dy, en, LV, q); #c1(FGFBP2) c2(NP_114156) c3(fibroblast growth factor-binding protein 2 precursor) c4(222412) c5(222413, 222414, 222415, 222416, 222417, 222418, 222419, 222420) c6(222421, 222422) c7(ci, m); #c1(FGFBP3) c2(NP_689642) c3(fibroblast growth factor-binding protein 3 precursor) c4(243393) c5(243394, 243395, 243396, 243397, 243398, 243399, 243400, 243401) c6(243402, 243403) c7(hq); #c1(FGFR1) c2(NP_001167535) c3(fibroblast growth factor receptor 1 isoform fl precursor) c4(69008) c5(69009, 69010, 69011, 69012, 69013, 69014) c6( )c7(bM, T, bO, fd, amj, ci, mF, ip, x, Vp, bv, Na, aE, arA, il, btA, el, aTq, bjy, iE, pr, aW, aVQ, Az, btB, g, as, eE, eF, cm, aiv, zV, iP, y, aMP, eM, nB, BK, oy, PV, sv, nw, k, m, Xu, in, ik, btv, n, oY, ct, btF, yn, btm, ft, bti, bto, amJ, dl, wb, Wr, btr, Nr, yi, avP, sp, hQ, btj, aq, btp, kp, po, op, bty, Qy, YE, btn, bP, dB, Qu, eD, avF, anx, B, bL, fM, aA, cz, v, j, q, btx, eA, UE, aT, bts, dz, btE, Nc, jj, ae, af, dl, bA, btz, kl, wr, il, Y, btq, aeS, r, en, t, KQ, iK, aoy, ck, baz, kP, mG, JL, UF, bt, bjX, id, jh, btu, Fb, zU, EA, hF, ad, fU, btD, btw, hd, av, lQ, nJ, btt, d, et, vj, ff, btC, eH, cr, af, jb, bz, Hp, Nq, z, aKC, lC, iu, sH, DB); #c1(FGFR1) c2(NP_001167538) c3(fibroblast growth factor receptor 1 isoform 14 precursor) c4(69015) c5(69016, 69017, 69018, 69019, 69020, 69021, 69022, 69023, 69024, 69025, 69026, 69027, 69028, 69029, 69030, 69031, 69032, 69033, 69034, 69035, 69036, 69037, 69038, 69039, 69040, 69041) c6( )c7(bM, T, bO, fd, amj, ci, mF, ip, x, Vp, bv, Na, aE, arA, il, btA, eL, aTq, bjy, iE, pr, aW, aVQ, Az, btB, g, as, eE, eF, cm, aiv, zV, iP, y, aMP, eM, nB, BK, oy, PV, sv, nw, k, m, Xu, in, ik, btv, n, oY, ct, btF, yn, btm, ft, bti, bto, amJ, dL, wb, Wr, btr, Nr, yi, avP, sp, hQ, btj, aq, btp, kp, po, op, bty, Qy, YE, btn, bP, dB, Qu, eD, avF, anx, B, bL, fM, aA, cz, v, j, q, btx, eA, UE, aT, bts, dz, btE, Nc, jj, ae, af, dl, bA, btz, kl, wr, iU, Y, btq, aeS, r, en, t, KQ, ik, aoy, ck, baz, kP, mG, JL, UF, bt, bjX, id, jh, btu, Fb, zU, EA, hF, ad, fU, btD, btw, hd, av, IQ, nJ, btt, d, et, vj, ff, btC, eH, cr, aF, jb, bz, Hp, Nq, z, akC, lC, iu, sH, DB); #c1(FGFR1) c2(NP_056934) c3(fibroblast growth factor receptor 1 isoform 2 precursor) c4(188151, 188152) c5( )c6( )c7(bM, T, bO, fd, amj, ci, mF, ip, x, Vp, bv, Na, aE, arA, il, btA, eL, aTq, bjy, iE, pr, aW, aVQ, Az, btB, g, as, eE, eF, cm, aiv, zV, iP, y, aMP, eM, nB, BK, oy, PV, sv, nw, k, m, Xu, in, ik, btv, n, oY, ct, btF, yn, btm, ft, bti, bto, amJ, dL, wb, Wr, btr, Nr, yi, avP, sp, hQ, btj, aq, btp, kp, po, op, bty, Qy, YE, btn, bP, dB, Qu, eD, avF, anx, B, bL, fM, aA, cz, v, j, q, btx, eA, UE, aT, bts, dz, btE, Nc, jj, ae, af, dl, bA, btz, kl, wr, iU, Y, btq, aeS, r, en, t, KQ, iK, aoy, ck, baz, kP, mG, JL, UF, bt, bjX, id, jh, btu, Fb, zU, EA, hF, ad, fU, btD, btw, hd, av, lQ, nJ, btt, d, et, vj, ff, btC, eH, cr, aF, jb, bz, Hp, Nq, z, aKC, lC, iu, sH, DB); #c1(FGFR1) c2(NP_075593) c3(fibroblast growth factor receptor 1 isoform 3 precursor) c4(214021, 214022) c5(214023, 214024, 214025, 214026, 214027, 214028, 214029, 214030, 214031, 214032) c6( )c7(bM, T, bO, fd, amj, ci, mF, ip, x, Vp, bv, Na, aE, arA, il, btA, eL, aTq, bjy, iE, pr, aW, aVQ, Az, btB, g, as, BE, eF, cm, aiv, zV, iP, y, aMP, eM, nB, BK, oy, PV, sv, nw, k, m, Xu, in, ik, btv, n, oY, ct, btF, yn, btm, ft, bti, bto, amJ, dl, wb, Wr, btr, Nr, yi, avP, sp, hQ, btj, aq, btp, kp, po, op, bty, Qy, YE, btn, bP, dB, Qu, eD, avF, anx, B, bL, fM, aA, cz, v, j, q, btx, eA, UE, aT, bts, dz, btE, Nc, jj, ae, af, dl, bA, btz, kl, wr, il, Y, btq, aeS, r, en, t, KQ, iK, aoy, ck, baz, kP, mG, JL, UF, bt, bjX, id, jh, btu, Fb, zU, EA, hF, ad, fU, btD, btw, hd, av, IQ, nJ, btt, d, et, vj, ff, btC, eH, cr, af, jb, bz, Hp, Nq, z, akC, lC, iu, sH, DB); #c1(FGFR1) c2(NP_075594) c3(fibroblast growth factor receptor 1 isoform 4 precursor) c4(214033, 214034, 214035, 214036) c5(214037, 214038, 214039, 214040, 214041, 214042, 214043, 214044, 214045, 214046, 214047, 214048, 214049, 214050, 214051) c6( )c7(bM, T, bO, fd, amj, ci, mF, ip, x, Vp, bv, Na, aE, arA, il, btA, eL, aTq, bjy, iE, pr, aW, aVQ, Az, btB, g, as, eE, eF, cm, aiv, zV, iP, y, aMP, eM, nB, BK, oy, PV, sv, nw, k, m, Xu, in, ik, btv, n, oY, ct, btF, yn, btm, ft, bti, bto, amJ, dL, wb, Wr, btr, Nr, yi, avP, sp, hQ, btj, aq, btp, kp, po, op, bty, Qy, YE, btn, bP, dB, Qu, eD, avF, anx, B, bL, fM, aA, cz, v, j, q, btx, eA, UE, aT, bts, dz, btE, Nc, jj, ae, af, dl, bA, btz, kl, wr, iU, Y, btq, aeS, r, en, t, KQ, iK, aoy, ck, baz, kP, mG, JL, UF, bt, bjX, id, jh, btu, Fb, zU, EA, hF, ad, fU, btD, btw, hd, av, lQ, nJ, btt, d, et, vj, ff, btC, eH, cr, aF, jb, bz, Hp, Nq, z, akC, lC, iu, sH, DB); #c1(FGFR1) c2(NP_075598) c3(fibroblast growth factor receptor 1 isoform 1 precursor) c4(214052, 214053, 214054) c5( )c6( )c7(bM, T, bO, fd, amj, ci, mF, ip, x, Vp, bv, Na, aE, arA, il, btA, eL, aTq, bjy, iE, pr, aW, aVQ, Az, btB, g, as, eE, eF, cm, aiv, zV, iP, y, aMP, eM, nB, BK, oy, PV, sv, nw, k, m, Xu, in, ik, btv, n, oY, ct, btF, yn, btm, ft, bti, bto, amJ, dL, wb, Wr, btr, Nr, yi, avP, sp, hQ, btj, aq, btp, kp, po, op, bty, Qy, YE, btn, bP, dB, Qu, eD, avF, anx, B, bL, fM, aA, cz, v, j, q, btx, eA, UE, aT, bts, dz, btE, Nc, jj, ae, af, dl, bA, btz, kl, wr, iU, Y, btq, aeS, r, en, t, KQ, iK, aoy, ck, baz, kP, mG, JL, UF, bt, bjX, id, jh, btu, Fb, zU, EA, hF, ad, fU, btD, btw, hd, av, lQ, nJ, btt, d, et, vj, ff, btC, eH, cr, aF, jb, bz, Hp, Nq, z, aKC, lC, iu, sH, DB); #c1 (FGFR1) c2(XP_006716368) c3(fibroblast growth factor receptor 1 isoform X6) c4( )c5(290269, 290270, 290271, 290272, 290273) c6( )c7(bM, T, bO, fd, amj, ci, mF, ip, x, Vp, bv, Na, aE, arA, il, btA, eL, aTq, bjy, iE, pr, aW, aVQ, Az, btB, g, as, eE, eF, cm, aiv, zV, iP, y, aMP, eM, nB, BK, oy, PV, sv, nw, k, m, Xu, in, ik, btv, n, oY, ct, btF, yn, btm, ft, bti, bto, amJ, dl, wb, Wr, btr, Nr, yi, avP, sp, hQ, btj, aq, btp, kp, po, op, bty, Qy, YE, btn, bP, dB, Qu, eD, avF, anx, B, bL, fM, aA, cz, v, j, q, btx, eA, UE, aT, bts, dz, btE, Nc, jj, ae, af, dl, bA, btz, kl, wr, il, Y, btq, aeS, r, en, t, KQ, iK, aoy, ck, baz, kP, mG, JL, UF, bt, bjX, id, jh, btu, Fb, zU, EA, hF, ad, fU, btD, btw, hd, av, lQ, nJ, btt, d, et, vj, ff, btC, eH, cr, aF, jb, bz, Hp, Nq, z, aKC, lC, iu, sH, DB); #c1(FGFR1) c2(XP_011542748) c3(fibroblast growth factor receptor 1 isoform X4) c4(341336) c5(341337, 341338, 341339, 341340, 341341, 341342, 341343) c6( )c7(bM, T, bO, fd, amj, ci, mF, ip, x, Vp, bv, Na, aE, arA, il, btA, eL, aTq, bjy, iE, pr, aW, aVQ, Az, btB, g, as, eE, eF, cm, aiv, zV, iP, y, aMP, eM, nB, BK, oy, PV, sv, nw, k, m, Xu, in, ik, btv, n, oY, ct, btF, yn, btm, ft, bti, bto, amJ, dL, wb, Wr, btr, Nr, yi, avP, sp, hQ, btj, aq, btp, kp, po, op, bty, Qy, YE, btn, bP, dB, Qu, eD, avF, anx, B, bL, fM, aA, cz, v, j, q, btx, eA, UE, aT, bts, dz, btE, Nc, jj, ae, af, dl, bA, btz, kl, wr, iU, Y, btq, aeS, r, en, t, KQ, iK, aoy, ck, baz, kP, mG, JL, UF, bt, bjX, id, jh, btu, Fb, zU, EA, hF, ad, fU, btD, btw, hd, av, lQ, nJ, btt, d, et, vj, ff, btC, eH, cr, aF, jb, bz, Hp, Nq, z, akC, lC, iu, sH, DB); #c1(FGFR1) c2(XP_011542749) c3(fibroblast growth factor receptor 1 isoform X5) c4(341344) c5(341345, 341346, 341347, 341348, 341349, 341350) c6( )c7(bM, T, bO, fd, amj, ci, mF, ip, x, Vp, bv, Na, aE, arA, il, btA, eL, aTq, bjy, iE, pr, aW, aVQ, Az, btB, g, as, eE, eF, cm, aiv, zV, iP, y, aMP, eM, nB, BK, oy, PV, sv, nw, k, m, Xu, in, ik, btv, n, oY, ct, btF, yn, btm, ft, bti, bto, amJ, dl, wb, Wr, btr, Nr, yi, avP, sp, hQ, btj, aq, btp, kp, po, op, bty, Dy, YE, btn, bP, dB, Qu, eD, avF, anx, B, bL, fM, aA, cz, v, j, q, btx, eA, UE, aT, bts, dz, btE, Nc, jj, ae, af, dl, bA, btz, kl, wr, iU, Y, btq, aeS, r, en, t, KQ, iK, aoy, ck, baz, kP, mG, JL, UF, bt, bjX, id, jh, btu, Fb, zU, EA, hF, ad, fU, btD, btw, hd, av, lQ, nJ, btt, d, et, vj, ff, btC, eH, cr, aF, jb, bz, Hp, Nq, z, aKC, lC, iu, sH, DB); #c1 (FGFR1) c2(XP_011542750) c3(fibroblast growth factor receptor 1 isoform X9) c4( )c5(341351) c6( )c7(bM, T, bO, fd, amj, ci, mF, ip, x, Vp, bv, Na, aE, arA, il, btA, eL, aTq, bjy, iE, pr, aW, aVQ, Az, btB, g, as, eE, eF, cm, aiv, zV, iP, y, aMP, eM, nB, BK, oy, PV, sv, nw, k, m, Xu, in, ik, btv, n, oY, ct, btF, yn, btm, ft, bti, bto, amJ, dL, wb, Wr, btr, Nr, yi, avP, sp, hQ, btj, aq, btp, kp, po, op, bty, Qy, YE, btn, bP, dB, Qu, eD, avF, anx, B, bL, fM, aA, cz, v, j, q, btx, eA, UE, aT, bts, dz, btE, Nc, jj, ae, af, dl, bA, btz, kl, wr, iU, Y, btq, aeS, r, en, t, KQ, iK, aoy, ck, baz, kP, mG, JL, UF, bt, bjX, id, jh, btu, Fb, zU, EA, hF, ad, fU, btD, btw, hd, av, lO, nJ, btt, d, et, vj, ff, btC, eH, cr, aF, jb, bz, Hp, Nq, z, akC, lC, iu, sH, DB); #c1(FGFR1) c2(XP_011542752) c3(fibroblast growth factor receptor 1 isoform X11) c4(341352) c5(341353, 341354, 341355, 341356, 341357, 341358, 341359, 341360, 341361, 341362, 341363, 341364, 341365, 341366, 341367, 341368) c6( )c7(bM, T, bO, fd, amj, ci, mF, ip, x, Vp, bv, Na, aE, arA, il, btA, eL, aTq, bjy, iE, pr, aW, aVQ, Az, btB, g, as, BE, eF, cm, aiv, zV, iP, y, aMP, eM, nB, BK, oy, PV, sv, nw, k, m, Xu, in, ik, btv, n, oY, ct, btF, yn, btm, ft, bti, bto, amJ, dl, wb, Wr, btr, Nr, yi, avP, sp, hQ, btj, aq, btp, kp, po, op, bty, Qy, YE, btn, bP, dB, Qu, eD, avF, anx, B, bL, fM, aA, cz, v, j, q, btx, eA, UE, aT, bts, dz, btE, Nc, jj, ae, af, dl, bA, btz, kl, wr, iU, Y, btq, aeS, r, en, t, KQ, iK, aoy, ck, baz, kP, mG, JL, UF, bt, bjX, id, jh, btu, Fb, zU, EA, hF, ad, fU, btD, btw, hd, av, lQ, nJ, btt, d, et, vj, ff, btC, eH, cr, aF, jb, bz, Hp, Nq, z, akC, lC, iu, sH, DB); #c1(FGFR1) c2(XP_011542754) c3(fibroblast growth factor receptor 1 isoform X15) c4(341369) c5(341370, 341371, 341372, 341373, 341374, 341375, 341376, 341377, 341378, 341379, 341380, 341381, 341382, 341383, 341384, 341385, 341386, 341387, 341388) c6( )c7(bM, T, bO, fd, amj, ci, mF, ip, x, Vp, bv, Na, aE, arA, il, btA, eL, aTq, bjy, iE, pr, aW, aVQ, Az, btB, g, as, eE, eF, cm, aiv, zV, iP, y, aMP, eM, nB, BK, oy, PV, sv, nw, k, m, Xu, in, ik, btv, n, oY, ct, btF, yn, btm, ft, bti, bto, amJ, dL, wb, Wr, btr, Nr, yi, avP, sp, hl, btj, aq, btp, kp, po, op, bty, Qy, YE, btn, bP, dB, Qu, eD, avF, anx, B, bL, fM, aA, cz, v, j, q, btx, eA, UE, aT, bts, dz, btE, Nc, jj, ae, af, dl, bA, btz, kl, wr, il, Y, btq, aeS, r, en, t, KQ, ik, aoy, ck, baz, kP, mG, JL, UF, bt, bjX, id, jh, btu, Fb, zU, EA, hF, ad, fU, btD, btw, hd, av, lQ, nJ, btt, d, et, vj, ff, btC, eH, cr, aF, jb, bz, Hp, Nq, z, akC, lC, iu, sH, DB); #c1(FGFR10P) c2(NP_001265619) c3(FGFR1 oncogene partner isoform c) c4(99028) c5(99029, 99030, 99031, 99032, 99033, 99034) c6( )c7(av, iV, eM, et, w, HA, pg, dz, qj, ci, hf, bt, bz); #c1(FGFR10P) c2(NP_008976) c3(FGFR1 oncogene partner isoform a) c4(169987) c5(169988, 169989, 169990, 169991, 169992, 169993) c6( )c7(av, iV, eM, et, w, HA, pg, dz, qj, ci, hf, bt, bz); #c1(FGFR10P) c2(NP_919410) c3(FGFR1 oncogene partner isoform b) c4(258539) c5(258540, 258541, 258542, 258543, 258544, 258545) c6( )c7(av, iV, eM, et, w, HA, pg, dz, qj, ci, hf, bt, bz); #c1(FGFR10P2) c2(NP_001165358) c3(FGFR1 oncogene partner 2 isoform 2) c4(67632, 67633) c5(67634, 67635, 67636, 67637, 67638, 67639) c6( )c7(et, dz, btG); #c1(FGFR10P2) c2(NP_001165359) c3(FGFR1 oncogene partner 2 isoform 3) c4(67640) c5(67641, 67642, 67643, 67644, 67645, 67646, 67647, 67648, 67649, 67650, 67651, 67652) c6( )c7(et, dz, btG); #c1(FGFR10P2) c2(NP_056448) c3(FGFR1 oncogene partner 2 isoform 1) c4(187588) c5(187589, 187590, 187591, 187592, 187593, 187594) c6( )c7(et, dz, btG); #c1(FGFR2) c2(NP_000132) c3(fibroblast growth factor receptor 2 isoform 1 precursor) c4(2887) c5(2888, 2889, 2890, 2891, 2892) c6( )c7(aOR, G, T, aC, jW, if, al, bO, fd, btM, aeu, bB, ci, sD, YD, Na, aE, ta, K, hu, eL, li, yo, btH, zQ, btN, aW, are, Az, btB, Qm, agj, g, as, eF, cm, aiv, aj, zV, bst, eM, nB, lp, AM, iS, cX, uJ, k, m, bsB, ik, iu, bnL, QO, zt, Pi, qy, btv, n, aln, nA, qN, BM, acy, rc, yn, md, btm, jz, bto, amJ, agi, btl, h, eV, dL, wb, Wr, he, Gc, sp, btj, Pp, kp, yq, btL, btK, bP, Qu, hk, hL, hR, avF, zG, art, bD, it, B, btf, aGn, yk, eX, aA, cz, bsx, q, eA, bke, xx, aHu, afQ, aT, bts, rD, btE, bK, Nc, ae, af, dl, eC, qw, avg, btz, vT, Y, r, lo, eS, en, bu, cl, t, btO, ik, sS, rR, au, JL, btJ, bt, LS, gx, Mb, kh, id, bsv, acl, atS, Hn, btl, zu, iJ, ad, afb, bsz, dP, av, btw, hd, ib, db, bj, lQ, nJ, btP, btt, d, PE, ff, btC, eH, aF, atb, wW, gk, bE, adg, aed, VK, X, jr, bmY); #c1(FGFR2) c2(NP_001138385) c3(fibroblast growth factor receptor 2 isoform 3 precursor) c4(56495, 56496, 56497) c5(56498, 56499, 56500, 56501, 56502, 56503, 56504, 56505, 56506, 56507) c6( )c7(aDR, G, T, aC, jW, if, al, bO, fd, btM, aeu, bB, ci, sD, YD, Na, aE, ta, K, hu, eL, li, yo, btH, zQ, btN, aW, are, Az, btB, Qm, agj, g, as, eF, cm, aiv, aj, zV, bst, eM, nB, lp, AM, iS, cX, uJ, k, m, bsB, ik, iu, bnl, oO, zt, Pi, qy, btv, n, aln, nA, qN, BM, acy, rc, yn, md, btm, jz, bto, amJ, agi, btQ, h, eV, dL, wb, Wr, he, Gc, sp, btj, Pp, kp, yq, btl, btk, bP, Qu, hk, hL, hR, avF, zG, art, bD, it, B, btf, aGn, yk, ex, aA, cz, bsx, q, eA, bke, xx, aHu, afd, aT, bts, rD, btE, bK, Nc, ae, af, dl, eC, qw, avg, btz, vT, Y, r, lo, eS, en, bu, cl, t, btO, iK, sS, rR, au, JL, btJ, bt, LS, gx, Mb, kh, id, bsv, acl, atS, Hn, btl, zu, iJ, ad, afb, bsz, dP, av, btw, hd, ib, db, bj, lQ, nJ, btP, btt, d, PE, ff, btC, eH, aF, atb, wW, gk, bE, adg, aed, VK, X, jr, bmY); #c1(FGFR2) c2(NP_001138386) c3(fibroblast growth factor receptor 2 isoform 4 precursor) c4(56508) c5(56509, 56510, 56511, 56512, 56513, 56514) c6( )c7(aDR, G, T, aC, jW, if, al, bO, fd, btM, aeu, bB, ci, sD, YD, Na, aE, ta, K, hu, eL, li, yo, btH, zQ, btN, aW, are, Az, btB, Qm, agj, g, as, eF, cm, aiv, aj, zV, bst, eM, nB, lp, AM, iS, cX, uJ, k, m, bsB, ik, iu, bnl, oO, zt, Pi, qy, btv, n, aln, nA, qN, BM, acy, rc, yn, md, btm, jz, bto, amJ, agi, btQ, h, eV, dL, wb, Wr, he, Gc, sp, btj, Pp, kp, yq, btL, btk, bP, Qu, hk, hL, hR, avF, zG, art, bD, it, B, btf, aGn, yk, eX, aA, cz, bsx, q, eA, bke, xx, aHu, afQ, aT, bts, rD, btE, bK, Nc, ae, af, dl, eC, qw, avg, btz, vT, Y, r, lo, eS, en, bu, cl, t, btO, ik, sS, rR, au, JL, btJ, bt, LS, gx, Mb, kh, id, bsv, acl, atS, Hn, btl, zu, iJ, ad, afb, bsz, dP, av, btw, hd, ib, db, bj, lQ, nJ, btP, btt, d, PE, ff, btC, eH, aF, atb, wW, gk, bE, adg, aed, VK, X, jr, bmY); #c1(FGFR2) c2(NP_001138387) c3(fibroblast growth factor receptor 2 isoform 5 precursor) c4(56515, 56516) c5(56517, 56518, 56519, 56520, 56521, 56522, 56523, 56524, 56525, 56526, 56527, 56528) c6( )c7(aDR, G, T, aC, jW, if, al, bO, fd, btM, aeu, bB, ci, sD, YD, Na, aE, ta, K, hu, eL, li, yo, btH, zQ, btN, aW, are, Az, btB, Om, agj, g, as, eF, cm, aiv, aj, zV, bst, eM, nB, lp, AM, iS, cX, uJ, k, m, bsB, ik, iu, bnl, QO, zt, Pi, qy, btv, n, aln, nA, qN, BM, acy, rc, yn, md, btm, jz, bto, amJ, agi, btQ, h, eV, dL, wb, Wr, he, Gc, sp, btj, Pp, kp, yq, btL, btk, bP, Qu, hk, hL, hR, avF, zG, art, bD, it, B, btf, aGn, yk, eX, aA, cz, bsx, q, eA, bke, xx, aHu, afQ, aT, bts, rD, btE, bK, Nc, ae, af, dl, eC, qw, avg, btz, vT, Y, r, lo, eS, en, bu, cl, t, btO, ik, sS, rR, au, JL, btJ, bt, LS, gx, Mb, kh, id, bsv, acl, atS, Hn, btl, zU, iJ, ad, afb, bsz, dP, av, btw, hd, ib, db, bj, lQ, nJ, btP, btt, d, PE, ff, btC, eH, aF, atb, wW, gk, bE, adg, aed, VK, X, jr, bmY); #c1(FGFR2) c2(NP_00138389) c3(fibroblast growth factor receptor 2 isoform 7 precursor) c4(56529) c5(56530, 56531, 56532, 56533, 56534, 56535, 56536, 56537, 56538, 56539, 56540) c6( )c7(aOR, G, T, aC, jW, if, al, bO, fd, btM, aeu, bB, ci, sD, YD, Na, aE, ta, K, hu, eL, li, yo, btH, zQ, btN, aW, are, Az, btB, Qm, agj, g, as, eF, cm, aiv, aj, zV, bst, eM, nB, lp, AM, iS, cX, uJ, k, m, bsB, ik, iu, bnl, 00, zt, Pi, qy, btv, n, aln, nA, qN, BM, acy, rc, yn, md, btm, jz, bto, amJ, agi, btQ, h, eV, dL, wb, Wr, he, Gc, sp, btj, Pp, kp, yq, btl, btk, bP, Qu, hk, hL, hR, avF, zG, art, bD, it, B, btf, aGn, yk, eX, aA, cz, bsx, q, eA, bke, xx, aHu, afQ, aT, bts, rD, btE, bK, Nc, ae, af, dl, eC, qw, avg, btz, vT, Y, r, lo, eS, en, bu, cl, t, btO, ik, sS, rR, au, JL, btJ, bt, LS, gx, Mb, kh, id, bsv, acl, atS, Hn, btl, zU, iJ, ad, afb, bsz, dP, av, btw, hd, ib, db, bj, lQ, nJ, btP, btt, d, PE, ff, btC, eH, aF, atb, wW, gk, bE, adg, aed, VK, X, jr, bmY); #c1(FGFR2) c2(NP_001138390) c3(fibroblast growth factor receptor 2 isoform 8 precursor) c4(56541) c5(56542, 56543, 56544, 56545, 56546, 56547) c6( )c7(aOR, G, T, aC, jW, if, al, bO, fd, btM, aeu, bB, ci, sD, YD, Na, aE, ta, K, hu, eL, li, yo, btH, zQ, btN, aW, are, Az, btB, Qm, agj, g, as, eF, cm, aiv, aj, zV, bst, eM, nB, lp, AM, iS, cX, uJ, k, m, bsB, ik, iu, bnl, oO, zt, Pi, qy, btv, n, aln, nA, qN, BM, acy, rc, yn, md, btm, jz, bto, amJ, agi, btQ, h, eV, dl, wb, Wr, he, Gc, sp, btj, Pp, kp, yq, btL, btk, bP, Qu, hk, hL, hR, avF, zG, art, bD, it, B, btf, aGn, yk, eX, aA, cz, bsx, q, eA, bke, xx, aHu, afQ, aT, bts, rD, btE, bK, Nc, ae, af, dl, eC, qw, avg, btz, vT, Y, r, lo, eS, en, bu, cl, t, btO, ik, sS, rR, au, JL, btJ, bt, LS, gx, Mb, kh, id, bsv, acl, atS, Hn, btl, zU, iJ, ad, afb, bsz, dP, av, btw, hd, ib, db, bj, lQ, nJ, btP, btt, d, PE, ff, btC, eH, af, atb, wW, gk, bE, adg, aed, VK, X, jr, bmY); #c1(FGFR2) c2(NP_001138391) c3(fibroblast growth factor receptor 2 isoform 9 precursor) c4(56548) c5(56549, 56550, 56551, 56552, 56553) c6( )c7(aDR, G, T, aC, jW, if, al, bO, fd, btM, aeu, bB, ci, sD, YD, Na, aE, ta, K, hu, eL, li, yo, btH, zQ, btN, aW, are, Az, btB, lm, agj, g, as, eF, cm, aiv, aj, zV, bst, eM, nB, lp, AM, iS, cX, uJ, k, m, bsB, ik, iu, bnl, QO, zt, Pi, qy, btv, n, aln, nA, qN, BM, acy, rc, yn, md, btm, jz, bto, amJ, agi, btQ, h, eV, dL, wb, Wr, he, Gc, sp, btj, Pp, kp, yq, btL, btk, bP, Qu, hk, hL, hR, avF, zG, art, bD, it, B, btf, aGn, yk, eX, aA, cz, bsx, q, eA, bke, xx, aHu, afQ, aT, bts, rD, btE, bK, Nc, ae, af, dl, eC, qw, avg, btz, vT, Y, r, lo, eS, en, bu, cl, t, btO, ik, sS, rR, au, JL, btJ, bt, LS, gx, Mb, kh, id, bsv, acl, atS, Hn, btl, zu, iJ, ad, afb, bsz, dP, av, btw, hd, ib, db, bj, lQ, nJ, btP, btt, d, PE, ff, btC, eH, aF, atb, wW, gk, bE, adg, aed, VK, X, jr, bmY); #c1(FGFR2) c2(NP_075259) c3(fibroblast growth factor receptor 2 isoform 2 precursor) c4(213768) c5(213769, 213770, 213771, 213772, 213773) c6( )c7(aDR, G, T, aC, jW, if, al, bO, fd, btM, aeu, bB, ci, sD, YD, Na, aE, ta, K, hu, eL, li, yo, btH, zQ, btN, aW, are, Az, btB, lm, agj, g, as, eF, cm, aiv, aj, zV, bst, eM, nB, lp, AM, iS, cX, uJ, k, m, bsB, ik, iu, bnl, QO, zt, Pi, qy, btv, n, aln, nA, qN, BM, acy, rc, yn, md, btm, jz, bto, amJ, agi, btQ, h, eV, dL, wb, Wr, he, Gc, sp, btj, Pp, kp, yq, btl, btk, bP, Qu, hk, hL, hR, avF, zG, art, bD, it, B, btf, aGn, yk, eX, aA, cz, bsx, q, eA, bke, xx, aHu, afQ, aT, bts, rD, btE, bK, Nc, ae, af, dl, eC, qw, avg, btz, vT, Y, r, lo, eS, en, bu, cl, t, btO, ik, sS, rR, au, JL, btJ, bt, LS, gx, Mb, kh, id, bsv, acl, atS, Hn, btl, zu, iJ, ad, afb, bsz, dP, av, btw, hd, ib, db, bj, lQ, nJ, btP, btt, d, PE, ff, btC, eH, aF, atb, wW, gk, bE, adg, aed, VK, X, jr, bmY); #c1(FGFR2) c2(XP_0067 (7771) c3(fibroblast growth factor receptor 2 isoform X1) c4( )c5(291322) c6( ) c7(aOR, G, T, aC, jW, if, al, bO, fd, btM, aeu, bB, ci, sD, YD, Na, aE, ta, K, hu, eL, li, yo, btH, zQ, btN, aW, are, Az, btB, Um, agj, g, as, eF, cm, aiv, aj, zV, bst, eM, nB, lp, AM, iS, cX, uJ, k, m, bsB, ik, iu, bnl, QO, zt, Pi, qy, btv, n, aln, nA, qN, BM, acy, rc, yn, md, btm, jz, bto, amJ, agi, btQ, h, eV, dL, wb, Wr, he, Gc, sp, btj, Pp, kp, yq, btL, btk, bP, Qu, hk, hL, HR, avF, zG, art, bD, it, B, btf, aGn, yk, eX, aA, cz, bsx, q, eA, bke, xx, aHu, afQ, aT, bts, rD, btE, bK, Nc, ae, af, dl, eC, qw, avg, btz, vT, Y, r, lo, eS, en, bu, cl, t, btO, iK, sS, rR, au, JL, btJ, bt, LS, gx, Mb, kh, id, bsv, acl, atS, Hn, btl, zU, iJ, ad, afb, bsz, dP, av, btw, hd, ib, db, bj, lQ, nJ, btP, btt, d, PE, ff, btC, eH, aF, atb, wW, gk, bE, adg, aed, VK, X, jr, bmY); #c1(FGFR2) c2(XP_0067 (7772) c3(fibroblast growth factor receptor 2 isoform X2) c4(291323, 291324, 291325) c5(291326, 291327, 291328, 291329, 291330, 291331, 291332, 291333, 291334, 291335, 291336, 291337, 291338) c6( )c7(aDR, G, T, aC, jW, if, al, bO, fd, btM, aeu, bB, ci, sD, YD, Na, aE, ta, K, hu, eL, li, yo, btH, zQ, btN, aW, are, Az, btB, Qm, agj, g, as, eF, cm, aiv, aj, zV, bst, eM, nB, lp, AM, iS, cX, uJ, k, m, bsB, ik, iu, bnl, oO, zt, Pi, qy, btv, n, aln, nA, qN, BM, acy, rc, yn, md, btm, jz, bto, amJ, agi, btl, h, eV, dL, wb, Wr, he, Gc, sp, btj, Pp, kp, yq, btL, btk, bP, Qu, hk, hL, hR, avF, zG, art, bD, it, B, btf, aGn, yk, eX, aA, cz, bsx, q, eA, bke, xx, aHu, afQ, aT, bts, rD, btE, bK, Nc, ae, af, dl, eC, qw, avg, btz, vT, Y, r, lo, eS, en, bu, cl, t, btO, iK, sS, rR, au, JL, btJ, bt, LS, gx, Mb, kh, id, bsv, acl, atS, Hn, btl, zU, iJ, ad, afb, bsz, dP, av, btw, hd, ib, db, bj, lQ, nJ, btP, btt, d, PE, ff, btC, eH, aF, atb, wW, gk, bE, adg, aed, VK, X, jr, bmY); #c1(FGFR2) c2(XP_0067/7773) c3 (fibroblast growth factor receptor 2 isoform X3) c4( )c5(291339, 291340) c6( )c7(aOR, G, T, aC, jW, if, al, bO, fd, btM, aeu, bB, ci, sD, YD, Na, aE, ta, K, hu, eL, li, yo, btH, zQ, btN, aW, are, Az, btB, Qm, agj, g, as, eF, cm, aiv, aj, zV, bst, eM, nB, lp, AM, iS, cx, uJ, k, m, bsB, ik, iu, bnl, oO, zt, Pi, qy, btv, n, aln, nA, qN, BM, acy, rc, yn, md, btm, jz, bto, amJ, agi, btQ, h, eV, dL, wb, Wr, he, Gc, sp, btj, Pp, kp, yq, btL, btk, bP, Qu, hk, hL, hR, avF, zG, art, bD, it, B, btf, aGn, yk, eX, aA, cz, bsx, q, eA, bke, xx, aHu, afQ, aT, bts, rD, btE, bK, Nc, ae, af, dl, eC, qw, avg, btz, vT, Y, r, lo, eS, en, bu, cl, t, btO, ik, sS, rR, au, JL, btJ, bt, LS, gx, Mb, kh, id, bsv, acl, atS, Hn, btl, zU, iJ, ad, afb, bsz, dP, av, btw, hd, ib, db, bj, lQ, nJ, btP, btt, d, PE, ff, btC, eH, af, atb, wW, gk, bE, adg, aed, VK, X, jr, bmY); #c1(FGFR2) c2(XP_006717774) c3(fibroblast growth factor receptor 2 isoform X4) c4( )c5(291341) c6( )c7(aDR, G, T, aC, jW, if, al, bO, fd, btM, aeu, bB, ci, sD, YD, Na, aE, ta, K, hu, eL, li, yo, btH, zQ, btN, aW, are, Az, btB, Qm, agj, g, as, eF, cm, aiv, aj, zV, bst, eM, nB, lp, AM, iS, cX, uJ, k, m, bsB, ik, iu, bnl, QO, zt, Pi, qy, btv, n, aln, nA, qN, BM, acy, rc, yn, md, btm, jz, bto, amJ, agi, btl, h, eV, dL, wb, Wr, he, Gc, sp, btj, Pp, kp, yq, btL, btk, bP, Qu, hk, hL, hR, avF, zG, art, bD, it, B, btf, aGn, yk, eX, aA, cz, bsx, q, eA, bke, xx, aHu, afQ, aT, bts, rD, btE, bK, Nc, ae, af, dl, eC, qw, avg, btz, vT, Y, r, lo, eS, en, bu, cl, t, btO, ik, sS, rR, au, JL, btJ, bt, LS, gx, Mb, kh, id, bsv, acl, atS, Hn, btl, zU, iJ, ad, afb, bsz, dP, av, btw, hd, ib, db, bj, lQ, nJ, btP, btt, d, PE, ff, btC, eH, aF, atb, wW, gk, bE, adg, aed, VK, X, jr, bmY); #c1(FGFR2) c2(XP_0067 (7775) c3(fibroblast growth factor receptor 2 isoform X5) c4(291342) c5(291343, 291344, 291345, 291346, 291347, 291348) c6( )c7(aDR, G, T, aC, jW, if, al, bO, fd, btM, aeu, bB, ci, sD, YD, Na, aE, ta, K, hu, eL, li, yo, btH, zQ, btN, aW, are, Az, btB, Um, agj, g, as, eF, cm, aiv, aj, zV, bst, eM, nB, lp, AM, iS, cX, uJ, k, m, bsB, ik, iu, bnl, QO, zt, Pi, qy, btv, n, aln, nA, qN, BM, acy, rc, yn, md, btm, jz, bto, amJ, agi, btQ, h, eV, dL, wb, Wr, he, Gc, sp, btj, Pp, kp, yq, btL, btk, bP, Qu, hk, hL, hR, avF, zG, art, bD, it, B, btf, aGn, yk, eX, aA, cz, bsx, q, eA, bke, xx, aHu, afQ, aT, bts, rD, btE, bK, Nc, ae, af, dl, eC, qw, avg, btz, vT, Y, r, lo, eS, en, bu, cl, t, btO, ik, sS, rR, au, JL, btJ, bt, LS, gx, Mb, kh, id, bsv, acl, atS, Hn, btl, zU, iJ, ad, afb, bsz, dP, av, btw, hd, ib, db, bj, lQ, nJ, btP, btt, d, PE, ff, btC, eH, aF, atb, wW, gk, bE, adg, aed, VK, X, jr, bmY); #c1(FGFR2) c2(XP_0067 (7776) c3(fibroblast growth factor receptor 2 isoform X7) c4( )c5(291349, 291350, 291351, 291352, 291353, 291354, 291355) c6( )c7(aDR, G, T, aC, jW, if, al, bO, fd, btM, aeu, bB, ci, sD, YD, Na, aE, ta, K, hu, eL, li, yo, btH, zQ, btN, aW, are, Az, btB, Um, agj, g, as, eF, cm, aiv, aj, zV, bst, eM, nB, lp, AM, iS, cX, uJ, k, m, bsB, ik, iu, bnl, QO, zt, Pi, qy, btv, n, aln, nA, qN, BM, acy, rc, yn, md, btm, jz, bto, amJ, agi, btQ, h, eV, dL, wb, Wr, he, Gc, sp, btj, Pp, kp, yq, btL, btk, bP, Qu, hk, hL, hR, avF, zG, art, bD, it, B, btf, aGn, yk, eX, aA, cz, bsx, q, eA, bke, xx, aHu, afQ, aT, bts, rD, btE, bK, Nc, ae, af, dl, eC, qw, avg, btz, vT, Y, r, lo, eS, en, bu, cl, t, btO, ik, sS, rR, au, JL, btJ, bt, LS, gx, Mb, kh, id, bsv, acl, atS, Hn, btl, zU, iJ, ad, afb, bsz, dP, av, btw, hd, ib, db, bj, lQ, nJ, btP, btt, d, PE, ff, btC, eH, aF, atb, wW, gk, bE, adg, aed, VK, X, jr, bmY); #c1(FGFR3) c2(NP_000133) c3(fibroblast growth factor receptor 3 isoform 1 precursor) c4(2893) c5(2894, 2895, 2896, 2897, 2898, 2899, 2900, 2901, 2902, 2903) c6( )c7(T, aC, if, fd, aeu, ci, YD, ip, Na, bjt, K, eL, Zm, azh, btS, aW, agj, g, Qt, zV, bst, eM, bpY, cj, iu, btR, wu, aw, btv, n, fr, nA, qN, MT, axC, Bl, btm, zw, agi, Nn, dL, Wr, bub, ks, Og, Ld, btK, op, xA, bP, Qu, hk, btW, ln, art, NB, aNW, fM, yk, aA, btT, cz, btX, q, eA, xx, XX, lg, Vr, yg, aT, rD, btE, Nc, zp, rv, ae, af, jr, hM, iU, rh, Y, r, lo, en, bu, f, ik, ck, vK, sS, btU, mG, JL, aoJ, bsT, LR, hl, kh, id, btY, jh, buc, ade, ad, MS, db, yt, lQ, wm, bua, apw, d, bjs, ff, btC, Of, btV, aF, btZ, bz, dr, wW, es, amS, bE, Km, adg, ne, sH); #c1(FGFR3) c2(NP_001156685) c3(fibroblast growth factor receptor 3 isoform 3 precursor) c4(61836) c5(61837, 61838, 61839, 61840, 61841, 61842) c6( )c7(T, aC, if, fd, aeu, ci, YD, ip, Na, bjt, K, eL, Zm, azh, btS, aW, agj, g, Qt, zV, bst, eM, bpY, cj, iu, btR, wu, aw, btv, n, fr, nA, qN, MT, axC, Bl, btm, zw, agi, Nn, dL, Wr, bub, ks, Og, Ld, btk, op, xA, bP, Qu, hk, btW, ln, art, NB, aNW, fM, yk, aA, btT, cz, btX, q, eA, xx, XX, lg, Vr, yg, aT, rD, btE, Nc, zp, rv, ae, af, jr, hM, il, rh, Y, r, lo, en, bu, f, ik, ck, vK, sS, btU, mG, JL, aoJ, bsT, LR, hl, kh, id, btY, jh, buc, ade, ad, MS, db, yt, lQ, wm, bua, apw, d, bjs, ff, btC, Of, btV, aF, btZ, bz, dr, wW, es, amS, bE, Km, adg, ne, sH); #c1(FGFR3) c2(NP_075254) c3 (fibroblast growth factor receptor 3 isoform 2 precursor) c4(213731) c5(213732, 213733, 213734, 213735, 213736, 213737, 213738, 213739, 213740, 213741, 213742, 213743) c6( )c7(T, aC, if, fd, aeu, ci, YD, ip, Na, bjt, K, eL, Zm, azh, btS, aW, agj, g, Qt, zV, bst, eM, bpY, cj, iu, btR, wu, aw, btv, n, fr, nA, qN, MT, axC, Bl, btm, zw, agi, Nn, dL, Wr, bub, ks, Og, Ld, btK, op, xA, bP, Qu, hk, btW, ln, art, NB, aNW, fM, yk, aA, btT, cz, btX, q, eA, xx, XX, lg, Vr, yg, aT, rD, btE, Nc, zp, rv, ae, af, jr, hM, iU, rh, Y, r, lo, en, bu, f, ik, ck, vK, sS, btU, mG, JL, aoJ, bsT, LR, hl, kh, id, btY, jh, buc, ade, ad, MS, db, yt, lL, wm, bua, apw, d, bjs, ff, btC, Of, btV, aF, btZ, bz, dr, wW, es, amS, bE, Km, adg, ne, sH); #c1(FGFR3) c2(XP_006713932) c3(fibroblast growth factor receptor 3 isoform X2) c4(287428) c5( )c6( )c7(T, aC, if, fd, aeu, ci, YD, ip, Na, bjt, K, eL, Zm, azh, btS, aW, agj, g, Qt, zV, bst, eM, bpY, cj, iu, btR, wu, aw, btv, n, fr, nA, qN, MT, axC, Bl, btm, zw, agi, Nn, dL, Wr, bub, ks, Og, Ld, btk, op, xA, bP, Qu, hk, btW, ln, art, NB, aNW, fM, yk, aA, btT, cz, btX, q, eA, xx, XX, lg, Vr, yg, aT, rD, btE, Nc, zp, rv, ae, af, jr, hM, iU, rh, Y, r, lo, en, bu, f, ik, ck, vK, sS, btU, mG, JL, aoJ, bsT, LR, hl, kh, id, btY, jh, buc, ade, ad, MS, db, yt, lQ, wm, bua, apw, d, bjs, ff, btC, Of, btV, aF, btZ, bz, dr, wW, es, amS, bE, Km, adg, ne, sH); #c1(FGFR3) c2(XP_011511724) c3(fibroblast growth factor receptor 3 isoform X8) c4(302448) c5(302449, 302450, 302451, 302452, 302453, 302454) c6( )c7(T, aC, if, fd, aeu, ci, YD, ip, Na, bjt, K, eL, Zm, azh, btS, aW, agj, g, Qt, zV, bst, eM, bpY, cj, iu, btR, wu, aw, btv, n, fr, nA, qN, MT, axC, BI, btm, zw, agi, Nn, dL, Wr, bub, ks, Og, Ld, btK, op, xA, bP, Qu, hk, btW, ln, art, NB, aNW, fM, yk, aA, btT, cz, btX, q, eA, xx, XX, lg, Vr, yg, aT, rD, btE, Nc, zp, rv, ae, af, jr, hM, il, rh, Y, r, lo, en, bu, f, ik, ck, vK, sS, btU, mG, JL, aoJ, bsT, LR, hl, kh, id, btY, jh, buc, ade, ad, MS, db, yt, lQ, wm, bua, apw, d, bjs, ff, btC, Of, btV, aF, btZ, bz, dr, wW, es, amS, bE, Km, adg, ne, sH); #c1(FGFR4) c2(NP_001278909) c3(fibroblast growth factor receptor 4 isoform 3 precursor) c4( ) c5(110868, 110869, 110870, 110871, 110872, 110873, 110874, 110875, 110876, 110877, 110878, 110879, 110880, 110881, 110882, 110883, 110884, 110885, (10886, 110887) c6( )c7(G, T, pf, zs, al, fd, bB, ci, ip, x, bv, aE, K, eL, azh, yo, bbM, aW, g, as, eF, cm, aHd, eM, nB, iA, U, cj, OD, m, Xu, ik, iu, qy, n, aj, fr, ct, bud, h, dL, Dz, sp, Xg, aq, kp, cM, asE, hk, eD, hR, B, bL, fM, dA, yk, aDE, aA, cz, hy, v, j, bX, q, J, eA, afQ, qK, aT, dz, ae, jf, s, il, Y, r, en, cl, LV, t, iK, pb, aDQ, ck, sS, alC, Dy, gX, aDR, bt, aZ, btY, jh, bS, OH, ad, hX, av, hd, fb, d, asf, ff, aF, anW, Z, bz, dr, OG, bE, ag, ra, X); #c1(FGFR4) c2(NP_075252) c3(fibroblast growth factor receptor 4 isoform 2 precursor) c4(213719) c5(213720, 213721, 213722, 213723, 213724, 213725, 213726, 213727, 213728, 213729, 213730) c6( )c7(G, T, pf, zs, al, fd, bB, ci, ip, x, bv, aE, K, eL, azh, yo, bbM, aW, g, as, eF, cm, aHd, eM, nB, iA, U, cj, OD, m, Xu, ik, iu, qy, n, aj, fr, ct, bud, h, dL, Dz, sp, Xg, aq, kp, cM, asE, hk, eD, hR, B, bL, fM, dA, yk, aDE, aA, cz, hy, v, j, bX, q, J, eA, afQ, qK, aT, dz, ae, jf, s, il, Y, r, en, cl, LV, t, iK, pb, aDQ, ck, sS, alC, Dy, gX, aDR, bt, aZ, btY, jh, bS, OH, ad, hX, av, hd, fb, d, asf, ff, aF, anW, Z, bz, dr, OG, bE, ag, ra, X); #c1(FGFR4) c2(XP_005265895) c3(fibroblast growth factor receptor 4 isoform X1) c4(279824, 279825, 279826, 279827, 279828) c5(279829, 279830, 279831, 279832, 279833, 279834, 279835, 279836) c6( )c7(G, T, pf, zs, al, fd, bB, ci, ip, x, bv, aE, K, eL, azh, yo, bbM, aW, g, as, eF, cm, aHd, eM, nB, iA, U, cj, OD, m, Xu, ik, iu, qy, n, aj, fr, ct, bud, h, dL, Dz, sp, Xg, aq, kp, cM, asE, hk, eD, hR, B, bL, fM, dA, yk, aDE, aA, cz, hy, v, j, bX, q, J, eA, afd, qK, aT, dz, ae, jf, s, il, Y, r, en, cl, LV, t, iK, pb, aDQ, ck, sS, alC, Dy, gX, aDR, bt, aZ, btY, jh, bS, OH, ad, hX, av, hd, fb, d, asf, ff, aF, anW, Z, bz, dr, OG, bE, ag, ra, X); #c1(FGFR4) c2(XP_011532766) c3(fibroblast growth factor receptor 4 isoform X2) c4( )c5(328837, 328838, 328839, 328840, 328841, 328842) c6( )c7(G, T, pf, zs, al, fd, bB, ci, ip, x, bv, aE, K, eL, azh, yo, bbM, aW, g, as, eF, cm, aHd, eM, nB, iA, U, cj, OD, m, Xu, ik, iu, qy, n, aj, fr, ct, bud, h, dL, Dz, sp, Xg, aq, kp, cM, asE, hk, eD, hR, B, bL, fM, dA, yk, aDE, aA, cz, hy, v, j, bX, q, J, eA, afQ, qK, aT, dz, ae, jf, s, il, Y, r, en, cl, LV, t, ik, pb, aDQ, ck, sS, alC, Dy, gX, aDR, bt, aZ, btY, jh, bS, OH, ad, hX, av, hd, fb, d, asf, ff, aF, anW, Z, bz, dr, OG, bE, ag, ra, X); #c1(FGFR4) c2(XP_011532767) c3(fibroblast growth factor receptor 4 isoform X3) c4(328843) c5(328844, 328845, 328846, 328847, 328848, 328849) c6( )c7(G, T, pf, zs, al, fd, bB, ci, ip, x, bv, aE, K, eL, azh, yo, bbM, aW, g, as, eF, cm, aHd, eM, nB, iA, U, cj, OD, m, Xu, ik, iu, qy, n, aj, fr, ct, bud, h, dL, Dz, sp, Xg, aq, kp, cM, asE, hk, eD, hR, B, bL, fM, dA, yk, aDE, aA, cz, hy, v, j, bX, q, J, eA, af, qK, aT, dz, ae, jf, s, il, Y, r, en, cl, LV, t, iK, pb, aDQ, ck, sS, alC, Dy, gX, aDR, bt, aZ, btY, jh, bS, OH, ad, hX, av, hd, fb, d, asf, ff, aF, anW, Z, bz, dr, OG, bE, ag, ra, X); #c1(FGFRL1) c2(NP_001004358) c3(fibroblast growth factor receptor-like 1 precursor) c4(20619, 20620, 20621, 20622) c5(20623, 20624, 20625, 20626, 20627, 20628, 20629, 20630, 20631, 20632, 20633, 20634, 20635, 20636, 20637, 20638, 20639, 20640, 20641, 20642, 20643, 20644, 20645, 20646, 20647, 20648, 20649, 20650, 20651, 20652, 20653, 20654, 20655, 20656, 20657, 20658) c6( )c7(bM, T, aW, aT, it, dL, m, t, eL, axC); #c1(FGG) c2(NP_000500) c3(fibrinogen gamma chain isoform gamma-A precursor) c4(10454) c5(10455, 10456, 10457, 10458, 10459, 10460, 10461) c6( )c7(dD, ea, iW, e, aw, aA, cz, vr, j, eq, ad, dY, jk, M, cy, af, eu, fl, vs, aUF, ar, W, ac, wx, aS, dy, fD, aLL); #c1(FGG) c2(NP_068656) c3(fibrinogen gamma chain isoform gamma-B precursor) c4(209789) c5(209790, 209791, 209792, 209793, 209794, 209795, 209796, 209797, 209798, 209799, 209800, 209801, 209802, 209803, 209804, 209805, 209806, 209807, 209808, 209809, 209810, 209811, 209812, 209813, 209814, 209815, 209816, 209817, 209818, 209819, 209820, 209821, 209822, 209823, 209824, 209825, 209826, 209827, 209828, 209829, 209830, 209831, 209832, 209833, 209834, 209835, 209836, 209837, 209838, 209839, 209840, 209841, 209842, 209843, 209844, 209845, 209846, 209847, 209848, 209849, 209850, 209851, 209852, 209853, 209854, 209855, 209856, 209857, 209858, 209859, 209860, 209861, 209862, 209863, 209864, 209865, 209866, 209867, 209868, 209869, 209870, 209871, 209872, 209873, 209874, 209875, 209876, 209877, 209878, 209879, 209880, 209881, 209882, 209883, 209884, 209885, 209886, 209887, 209888, 209889, 209890, 209891, 209892, 209893, 209894, 209895, 209896, 209897, 209898, 209899, 209900, 209901, 209902, 209903, 209904, 209905, 209906, 209907, 209908, 209909, 209910, 209911, 209912, 209913, 209914, 209915, 209916, 209917, 209918, 209919, 209920, 209921, 209922, 209923, 209924, 209925, 209926, 209927, 209928, 209929, 209930, 209931, 209932, 209933) c6( )c7(dD, ea, iW, e, aw, aA, cz, vr, j, eq, ad, dY, jk, M, cy, af, eu, fQ, vs, aUF, ar, W, ac, wx, aS, dy, fD, aLL); #c1(FGGY) c2(XP_011540039) c3 (FGGY carbohydrate kinase domain-containing protein isoform X7) c4( )c5(338334, 338335, 338336, 338337, 338338, 338339) c6( )c7(bL, Vd, c); #c1(FGL1) c2(NP_004458) c3(fibrinogen-like protein 1 precursor) c4(146471, 146472) c5(146473, 146474, 146475, 146476, 146477, 146478, 146479, 146480, 146481, 146482, 146483, 146484, 146485, 146486, 146487, 146488, 146489, 146490, 146491, 146492, 146493, 146494, 146495, 146496, 146497, 146498, 146499, 146500, 146501, 146502, 146503, 146504, 146505, 146506, 146507, 146508, 146509, 146510, 146511, 146512, 146513, 146514, 146515, 146516, 146517, 146518, 146519, 146520, 146521, 146522, 146523, 146524, 146525, 146526, 146527, 146528, 146529, 146530) c6(146531, 146532) c7(T, KM, B, vi); #c1(FGL2) c2(NP_006673) c3(fibroleukin precursor) c4(167042) c5(167043, 167044, 167045, 167046, 167047, 167048) c6( )c7(T, fJ, uz, lo, jE, B, hX); #c1(FGR) c2(XP_011539313) c3(tyrosine-protein kinase Fgr isoform X2) c4( )c5(337382, 337383, 337384, 337385, 337386, 337387) c6( )c7(gx, aEQ, A, aW, EQ, aT, mg, PD, h, ci, bt, B); #c1(FGR) c2(XP_011539314) c3 (tyrosine-protein kinase Fgr isoform X3) c4(337388) c5( )c6( )c7(gx, aEQ, A, aW, EQ, aT, mg, PD, h, ci, bt, B); #c1(FGR) c2(XP_011539315) c3(tyrosine-protein kinase Fgr isoform X4) c4( )c5(337389, 337390, 337391, 337392, 337393, 337394) c6( )c7(gx, aEQ, A, aW, EQ, aT, mg, PD, h, ci, bt, B); #c1(FH) c2(NP_000134) c3 (fumarate hydratase, mitochondrial) c4(2904) c5(2905, 2906, 2907, 2908, 2909, 2910) c6( )c7(T, buf, fM, qb, vf, iW, jh, fr, jE, ci, bh, V, anu, ady, ad, er, zn, Zp, lS, x, cF, F, hw, py, hW, hb, amg, bue, dl, ae, bc, cY, aHE, aNC, iU, eZ, Hu, bu, ho, op, yR, ck, es, iA, vA, cj, k, anx, aps, bt); #c1(FHDC1) c2(NP_203751) c3(FH2 domain-containing protein 1) c4(228496) c5(228497, 228498, 228499, 228500, 228501, 228502, 228503, 228504, 228505, 228506) c6( )c7(c); #c1(FHIT) c2(NP_002003) c3(bis(5′-adenosyl)-triphosphatase) c4(123149) c5(123150, 123151, 123152, 123153, 123154, 123155, 123156, 123157, 123158, 123159, 123160, 123161, 123162, 123163, 123164, 123165, 123166, 123167, 123168, 123169, 123170) c6( )c7(T, os, aC, jW, fR, aEn, pf, if, al, WV, EQ, CH, bB, ci, ip, x, aE, lv, gs, K, hu, eL, xa, li, ayT, iE, aW, Ls, Ku, g, eF, cm, bw, qJ, eM, nB, iA, iT, buk, go, cj, azg, k, m, bul, ik, bui, sw, iB, aw, ht, n, fr, qN, cd, br, ajL, Bl, ft, cq, jz, aMT, h, auS, dL, Gw, pi, pl, aiB, AQ, fe, aq, yq, fq, Ld, Zj, hq, oU, kn, KT, Qy, YE, bP, dB, Qu, Pl, hL, lB, it, B, cn, bL, fM, oe, jx, fZ, aA, hy, j, Qo, AO, pd, q, er, eA, XX, oT, RI, lg, BJ, eO, aT, r, qv, Nc, Fx, Tj, ahg, jj, ae, jf, qw, bum, s, vT, il, oV, Y, ev, r, aYp, en, hf, cl, t, ik, ck, sS, ac, ig, nG, gX, oX, zu, bt, VB, hS, Gl, hl, id, akd, pY, em, jh, oZ, Fb, hF, buj, ad, qC, av, hd, ib, py, io, il, avQ, buh, auY, d, bug, cH, qt, ff, eH, aeV, aF, iV, gW, iR, bz, LB, es, bF, Ew, bE, afd, Gu, KS, X, jr); #c1(FHIT) c2(XP_011531788) c3(bis(5′-adenosyl)-triphosphatase isoform X2) c4(327873) c5(327874, 327875, 327876, 327877, 327878, 327879) c6( )c7(T, os, aC, jW, fR, aEn, pf, if, al, WV, EQ, CH, bB, ci, ip, x, aE, lv, gs, K, hu, eL, xa, li, ayT, iE, aW, Ls, Ku, g, eF, cm, bw, qJ, eM, nB, iA, iT, buk, go, cj, azg, k, m, bul, ik, bui, sw, iB, aw, ht, n, fr, qN, cd, br, ajL, Bl, ft, cq, jz, aMT, h, auS, dL, Gw, pi, pl, aiB, AQ, fe, aq, yq, fq, Ld, Zj, hq, OU, kn, KT, Qy, YE, bP, dB, Qu, Pl, hL, lB, it, B, cn, bL, fM, oe, jx, fZ, aA, hy, j, Qo, AD, pd, q, er, eA, XX, oT, RI, lg, BJ, eO, aT, rO, qv, Nc, Fx, Tj, ahg, jj, ae, jf, qw, bum, s, vT, iU, oV, Y, ev, r, aYp, en, hf, cl, t, ik, ck, sS, ac, ig, nG, gX, oX, zu, bt, VB, hS, Gl, hl, id, akd, pY, em, jh, oZ, Fb, hF, buj, ad, qC, av, hd, ib, py, io, il, avQ, buh, auY, d, bug, cH, qt, ff, eH, aeV, aF, iV, gW, iR, bz, LB, es, bF, Ew, bE, afd, Gu, KS, X, jr); #c1(FHL1) c2(NP_001153171) c3(four and a half LIM domains protein 1 isoform 5) c4(60492, 60493, 60494) c5(60495, 60496, 60497, 60498, 60499, 60500, 60501, 60502, 60503, 60504, 60505, 60506, 60507, 60508, 60509, 60510, 60511, 60512, 60513, 60514, 60515, 60516, 60517, 60518, 60519, 60520, 60521, 60522) c6( )c7(ID, T, bun, alx, yS, bur, aA, cz, fr, ci, BY, XW, cc, als, Ae, lC, oj, vc, azm, bl, db, ae, d, s, aW, xs, Pg, Ad, buq, ks, uY, aF, buo, alw, W, iA, bup, hc, Fw, adg, pJ, y, DH); #c1(FHL1) c2(NP_001153173) c3(four and a half LIM domains protein 1 isoform 3 precursor) c4(60523, 60524, 60525, 60526) c5(60527, 60528, 60529, 60530, 60531, 60532) c6( )c7(ID, T, bun, alx, yS, bur, aA, cz, fr, ci, BY, XW, cc, als, Ae, lC, oj, vc, azm, bl, db, ae, d, s, aW, xs, Pg, Ad, buq, ks, uY, aF, buo, alw, W, iA, bup, hc, Fw, adg, pJ, y, DH); #c1(FHL1) c2(NP_001153175) c3(four and a half LIM domains protein 1 isoform 4) c4( )c5(60533, 60534, 60535, 60536, 60537, 60538, 60539, 60540, 60541, 60542) c6( )c7(ID, T, bun, alx, yS, bur, aA, cz, fr, ci, BY, XW, cc, als, Ae, lC, oj, vc, azm, bl, db, ae, d, s, aW, xs, Pg, Ad, buq, ks, uY, aF, buo, alw, W, iA, bup, hc, Fw, adg, pJ, y, DH); #c1(FHL1) c2(XP_006724806) c3(four and a half LIM domains protein 1 isoform X3) c4(296342, 296343, 296344, 296345, 296346, 296347) c5(296348, 296349) c6( )c7(ID, T, bun, alx, yS, bur, aA, cz, fr, ci, BY, XW, cc, als, Ae, lC, oj, vc, azm, bl, db, ae, d, s, aW, xs, Pg, Ad, buq, ks, uY, aF, buo, alw, W, iA, bup, hc, Fw, adg, pJ, y, DH); #c1 (FHL1) c2(XP_006724809) c3 (four and a half LIM domains protein 1 isoform X2) c4( )c5(296350, 296351, 296352, 296353, 296354, 296355, 296356, 296357) c6( )c7(ID, T, bun, alx, yS, bur, aA, cz, fr, ci, BY, XW, cc, als, Ae, lC, oj, vc, azm, bl, db, ae, d, s, aW, xs, Pg, Ad, buq, ks, uY, aF, buo, alw, W, iA, bup, hc, Fw, adg, pJ, y, DH); #c1(FHL1) c2(XP_006724810) c3(four and a half LIM domains protein I isoform X1) c4( )c5(296358, 296359, 296360, 296361, 296362, 296363, 296364, 296365, 296366, 296367, 296368, 296369, 296370, 296371, 296372, 296373, 296374, 296375, 296376, 296377, 296378, 296379, 296380, 296381, 296382, 296383, 296384, 296385, 296386, 296387, 296388, 296389, 296390, 296391, 296392, 296393, 296394, 296395, 296396, 296397, 296398, 296399) c6( )c7(ID, T, bun, alx, yS, bur, aA, cz, fr, ci, BY, XW, cc, als, Ae, lC, oj, vc, azm, bl, db, ae, d, s, aW, xs, Pg, Ad, buq, ks, uY, aF, buo, alw, W, iA, bup, hc, Fw, adg, pJ, y, DH); #c1(FHL1) c2(XP_011529618) c3(four and a half LIM domains protein 1 isoform X4) c4(325145) c5(325146, 325147, 325148, 325149, 325150, 325151, 325152, 325153) c6( )c7(ID, T, bun, alx, yS, bur, aA, cz, fr, ci, BY, XW, cc, als, Ae, lC, oj, vc, azm, bl, db, ae, d, s, aW, xs, Pg, Ad, buq, ks, uY, aF, buo, alw, W, iA, bup, hc, Fw, adg, pJ, y, DH); #c1(FHL2) c2(XP_005263959) c3(four and a half LIM domains protein 2 isoform X2) c4(278583, 278584, 278585) c5(278586, 278587, 278588, 278589, 278590, 278591, 278592, 278593, 278594, 278595, 278596, 278597, 278598, 278599, 278600, 278601, 278602, 278603, 278604, 278605) c6( )c7(T, Dh, ha, al, cz, bB, j, ci, pz, q, ad, bv, yg, aT, cy, ae, d, s, aW, Y, eH, r, aq, iV, eF, cm, iR, KM, fq, ck, gk, bE, mx, gX, aS, bt, B); #c1(FHL2) c2(XP_005263963) c3(four and a half LIM domains protein 2 isoform X1) c4( )c5(278606, 278607, 278608, 278609, 278610, 278611, 278612, 278613, 278614, 278615, 278616, 278617, 278618, 278619, 278620, 278621, 278622, 278623, 278624, 278625, 278626, 278627, 278628, 278629) c6( )c7(T, Dh, ha, al, cz, bB, j, ci, pz, q, ad, bv, yg, aT, cy, ae, d, s, aW, Y, eH, r, aq, iV, eF, cm, iR, KM, fq, ck, gk, bE, mx, gX, aS, bt, B); #c1(FHL2) c2(XP_011509102) c3(four and a half LIM domains protein 2 isoform X3) c4(299685, 299686) c5(299687, 299688, 299689, 299690, 299691, 299692, 299693, 299694, 299695, 299696) c6( )c7(T, Dh, ha, al, cz, bB, j, ci, pz, q, ad, bv, yg, aT, cy, ae, d, s, aW, Y, eH, r, aq, iV, eF, cm, iR, KM, fq, ck, gk, bE, mx, gX, aS, bt, B); #c1(FHL5) c2(XP_006715676) c3(four and a half LIM domains protein 5 isoform X1) c4(289714, 289715) c5(289716, 289717, 289718, 289719, 289720, 289721, 289722, 289723, 289724, 289725, 289726, 289727, 289728, 289729, 289730, 289731, 289732) c6( )c7(bL, bM, T, gU, aw, j, ct, uu, eA, qH, AW, sO, gd, aT, dV, vH, iv, bK, an, af, A, s, ND, aW, AV, kQ, at, eg); #c1(FHOD1) c2(NP_037373) c3(FH1/FH2 domain-containing protein 1) c4(175097) c5(175098, 175099, 175100, 175101, 175102, 175103) c6( )c7(By, T); #c1(FHOD3) c2(NP_001268668) c3(FH1/FH2 domain-containing protein 3 isoform 2) c4(100113) c5(100114, 100115, 100116, 100117, 100118, 100119) c6( )c7(dl, cc, lB, vc, mx); #c1(FHOD3) c2(NP_079411) c3(FH1/FH2 domain-containing protein 3 isoform 1) c4(218005) c5(218006, 218007, 218008, 218009, 218010, 218011) c6( )c7(dQ, cc, lB, vc, mx); #c1(FHOD3) c2(XP_005258412) c3(FH1/FH2 domain-containing protein 3 isoform X12) c4(275648) c5(275649, 275650, 275651, 275652, 275653, 275654) c6( )c7(dl, cc, lB, vc, mx); #c1(FIBP) c2(NP_004205) c3(acidic fibroblast growth factor intracellular-binding protein isoform b) c4(144321) c5(144322, 144323, 144324, 144325, 144326, 144327) c6( )c7(al); #c1(FIBP) c2(NP_942600) c3(acidic fibroblast growth factor intracellular-binding protein isoform a) c4(260608) c5(260609, 260610, 260611, 260612, 260613, 260614) c6( )c7(al); #c1(FIG4) c2(NP_055660) c3(polyphosphoinositide phosphatase) c4(182580, 182581, 182582, 182583, 182584) c5(182585, 182586, 182587, 182588, 182589, 182590) c6( )c7(ql, c, Ol, but, axW, ha, agU, TQ, Lq, buu, fu, bus, bU, mF, bX, eA, cd, buv); #c1(FIG4) c2(XP_011534583) c3 (polyphosphoinositide phosphatase isoform X1) c4(331129) c5(331130, 331131, 331132, 331133, 331134, 331135, 331136, 331137, 331138, 331139, 331140, 331141, 331142, 331143, 331144, 331145, 331146, 331147, 331148, 331149, 331150, 331151, 331152, 331153, 331154, 331155, 331156, 331157, 331158, 331159) c6( )c7(ql, c, Ol, but, axW, ha, agU, TQ, Lq, buu, fu, bus, bu, mF, bX, eA, cd, buv); #c1(FIGF) c2(NP_004460) c3(vascular endothelial growth factor D preproprotein) c4(146533) c5(146534, 146535, 146536, 146537, 146538, 146539) c6( )c7(sY, T, fM, dD, aC, Tb, QO, sw, ea, e, Xi, jh, aA, eq, ci, fr, q, ad, eA, ft, hd, aE, py, aT, Vv, lG, io, dL, hB, eL, d, mR, s, Jl, aW, qS, ff, r, kp, aF, en, g, t, uC, bz, Bx, cm, LB, ck, pu, W, eM, nB, nM, iA, PN, lf, iu, y); #c1 (FIGLA) c2(NP_001004311) c3 (factor in the germline alpha) c4(20539) c5(20540, 20541, 20542, 20543, 20544, 20545) c6( )c7(il, zZ, buw); #c1(FIGN) c2(NP_060556) c3(fidgetin) c4(196546) c5(196547, 196548, 196549, 196550, 196551, 196552) c6( )c7(oe, aw, pN); #c1(FILIP1) c2(NP_001276916) c3(filamin-A-interacting protein 1 isoform 1) c4(108623) c5(108624) c6( )c7(aA); #c1(FILIP1) c2(NP_001287795) c3(filamin-A-interacting protein 1 isoform 3) c4(112890) c5(112891, 112892, 112893, 112894, 112895, 112896) c6( )c7(aA); #c1(FILIP1) c2(XP_005248770) c3(filamin-A-interacting protein 1 isoform XI) c4(268562) c5(268563, 268564, 268565, 268566, 268567, 268568, 268569, 268570, 268571, 268572, 268573) c6( )c7(aA); #c1 (FILIP1L) c2(NP_001035924) c3(filamin A-interacting protein 1-like isoform 3) c4(33405, 33406) c5(33407, 33408, 33409, 33410, 33411, 33412, 33413, 33414, 33415, 33416, 33417, 33418, 33419, 33420, 33421, 33422, 33423, 33424, 33425, 33426, 33427, 33428, 33429, 33430, 33431) c6( )c7(T, EQ, r, m, t, ae, q, ad); #c1(FILIP1L) c2(NP_001269723) c3(filamin A-interacting protein 1-like isoform 5) c4(102702, 102703) c5(102704, 102705, 102706, 102707, 102708, 102709, 102710, 102711, 102712, 102713, 102714, 102715, 102716, 102717, 102718, 102719) c6( )c7(T, EQ, r, m, t, ae, q, ad); #c1(FILIP1L) c2(NP_878913) c3(filamin A-interacting protein 1-like isoform 1) c4(257159) c5(257160, 257161, 257162, 257163, 257164, 257165, 257166) c6( )c7(T, EQ, r, m, t, ae, q, ad); #c1(FILIP1L) c2(XP_011510673) c3(filamin A-interacting protein I-like isoform X2) c4(301132, 301133, 301134) c5( )c6( )c7(T, EQ, r, m, t, ae, q, ad); #c1(FIPIL1) c2(NP_001128409) c3(pre-mRNA 3′-end-processing factor FIP1 isoform 2) c4(49970) c5(49971, 49972, 49973, 49974, 49975, 49976) c6( )c7(Nq, ck, aiu, Nr, T, Ta, il, pr, oy, qd, akC, apY, dz, po, Fu, fb, bt, op, yR, Tc); #c1(FIPIL1) c2(NP_001128410) c3(pre-mRNA 3′-end-processing factor FIP1 isoform 3) c4(49977) c5(49978, 49979, 49980, 49981, 49982, 49983) c6( )c7(Nq, ck, aiu, Nr, T, Ta, iU, pr, oy, qd, aKC, apY, dz, po, Fu, fb, bt, op, yR, Tc); #c1 (FIPIL1) c2(NP_112179) c3(pre-mRNA 3′-end-processing factor FIP1 isoform 1) c4(220646) c5(220647, 220648, 220649, 220650, 220651, 220652) c6( )c7(Nq, ck, aiu, Nr, T, Ta, il, pr, oy, qd, akC, apY, dz, po, Fu, fb, bt, op, yR, Tc); #c1(FIS1) c2(NP_057152) c3(mitochondrial fission 1 protein) c4(189166) c5(189167, 189168, 189169, 189170, 189171, 189172) c6( )c7(bt, bux, c); #c1(FITM1) c2(NP_981947) c3(fat storage-inducing transmembrane protein 1) c4(263177) c5(263178, 263179, 263180, 263181, 263182, 263183) c6( )c7(q, iT, r); #c1(FITM2) c2(NP_001073941) c3(fat storage-inducing transmembrane protein 2) c4(36635) c5(36636, 36637, 36638, 36639, 36640, 36641) c6( )c7(bL); #c1(FJX1) c2(NP_055159) c3(four-jointed box protein 1 precursor) c4(179519) c5(179520, 179521, 179522, 179523, 179524, 179525) c6( )c7(bB, bE, q, r); #c1(FKBP10) c2(NP_068758) c3(peptidyl-prolyl cis-trans isomerase FKBP10 precursor) c4(210202, 210203, 210204, 210205, 210206) c5(210207, 210208, 210209, 210210, 210211, 210212, 210213, 210214, 210215, 210216, 210217, 210218, 210219, 210220, 210221, 210222, 210223, 210224, 210225, 210226, 210227, 210228, 210229, 210230, 210231, 210232, 210233, 210234, 210235, 210236, 210237, 210238, 210239, 210240, 210241, 210242) c6( )c7(T, gU, alt, fG, buA, ci, PJ, q, aU, vH, K, zp, azh, buB, aRV, r, agF, t, buy, alC, buz, m); #c1(FKBP10) c2(XP_011523401) c3(peptidyl-prolyl cis-trans isomerase FKBP10 isoform X1) c4(317941) c5( )c6( )c7(T, gU, alt, fG, buA, ci, PJ, q, aU, vH, K, zp, azh, buB, aRV, r, agF, t, buy, alC, buz, m); #c1 (FKBP11) c2(NP_001137253) c3(peptidyl-prolyl cis-trans isomerase FKBP11 isoform 2) c4( )c5(55161, 55162, 55163, 55164, 55165) c6( )c7(PJ); #c1(FKBP11) c2(NP_001137254) c3(peptidyl-prolyl cis-trans isomerase FKBP11 isoform 3 precursor) c4(55166) c5(55167, 55168, 55169, 55170, 55171, 55172) c6( )c7(PJ); #c1(FKBP11) c2(NP_057678) c3(peptidyl-prolyl cis-trans isomerase FKBP11 isoform 1 precursor) c4(192317, 192318) c5(192319, 192320, 192321, 192322, 192323, 192324, 192325) c6( )c7(PJ); #c1(FKBP14) c2(NP_060416) c3(peptidyl-prolyl cis-trans isomerase FKBP14 precursor) c4(195844) c5(195845, 195846, 195847, 195848, 195849, 195850) c6( )c7(Eh, buC, PJ); #c1(FKBP15) c2(NP_056073) c3(FK506-binding protein 15) c4(185518, 185519, 185520) c5(185521, 185522, 185523, 185524, 185525, 185526, 185527, 185528, 185529, 185530, 185531, 185532) c6( )c7(IB, xa); #c1(FKBP15) c2(XP_006717081) c3(FK506-binding protein 15 isoform X1) c4( )c5(290706, 290707, 290708, 290709, 290710, 290711) c6( )c7(IB, xa); #c1(FKBP1A) c2(NP_000792) c3(peptidyl-prolyl cis-trans isomerase FKBP1A isoform a) c4(16073, 16074, 16075, 16076, 16077) c5(16078, 16079, 16080, 16081, 16082, 16083, 16084, 16085, 16086, 16087, 16088, 16089, 16090, 16091, 16092, 16093, 16094, 16095, 16096, 16097, 16098, 16099, 16100, 16101, 16102, 16103, 16104, 16105, 16106, 16107, 16108, 16109, 16110, 16111, 16112, 16113, 16114, 16115) c6( )c7(aiV, pe, aW, Ol, an, eZ, aT, akB, bK, lQ, app, PJ, bp, it, cd); #c1(FKBP1B) c2(NP_004107) c3(peptidyl-prolyl cis-trans isomerase FKBP1B isoform a) c4(143633) c5(143634, 143635, 143636, 143637, 143638, 143639, 143640) c6( )c7(qa, cc, cz, gZ, aj, PJ, cd, J); #c1(FKBP1B) c2(NP_473374) c3(peptidyl-prolyl cis-trans isomerase FKBP1B isoform b) c4(230494) c5(230495, 230496, 230497, 230498, 230499) c6( )c7(qa, cc, cz, gZ, aj, PJ, cd, J); #c1(FKBP2) c2(NP_004461) c3(peptidyl-prolyl cis-trans isomerase FKBP2 precursor) c4(146540, 146541) c5(146542, 146543, 146544, 146545, 146546, 146547, 146548, 146549, 146550, 146551, 146552, 146553, 146554, 146555, 146556, 146557, 146558, 146559, 146560, 146561) c6( )c7(T, aF, aA, jx, PJ); #c1(FKBP3) c2(NP_002004) c3(peptidyl-prolyl cis-trans isomerase FKBP3) c4(123171) c5(123172, 123173, 123174, 123175, 123176, 123177, 123178, 123179, 123180, 123181) c6( )c7(PJ); #c1(FKBP4) c2(NP_002005) c3(peptidyl-prolyl cis-trans isomerase FKBP4) c4(123182) c5(123183, 123184, 123185, 123186, 123187, 123188, 123189, 123190) c6(123191, 123192) c7(bM, T, aC, pf, wu, jx, bO, Nt, bS, ct, ci, PJ, ad, dP, x, Hx, aE, ws, dV, axc, aT, PD, h, K, wm, an, he, ae, af, Nr, aW, Y, lo, Pp, Xm, g, Z, PU, cm, iK, hq, Nq, pZ, cA, bE, Ei, pg, Le, B); #c1(FKBP5) c2(NP_001139247) c3(peptidyl-prolyl cis-trans isomerase FKBP5 isoform 1) c4(58969, 58970, 58971) c5(58972, 58973, 58974, 58975, 58976, 58977, 58978, 58979, 58980, 58981, 58982, 58983, 58984, 58985, 58986, 58987, 58988, 58989, 58990, 58991, 58992, 58993, 58994, 58995, 58996, 58997, 58998, 58999, 59000, 59001) c6( )c7(tJ, T, bM, jx, bO, Nt, aA, n, bS, gf, ct, ci, PJ, q, ad, eA, eJ, buD, dP, Hx, x, aE, ws, aT, h, aaw, bK, K, hK, he, ae, af, hZ, gj, Nr, aWm, Y, gl, lo, aF, po, g, Eg, PU, FX, cM, Z, iV, jb, FT, hq, Nq, ok, pZ, cA, abS, Ei, vl, pg, sv, pK, y, lB); #c1(FKBP5) c2(NP_001139249) c3(peptidyl-prolyl cis-trans isomerase FKBP5 isoform 2) c4(59002, 59003) c5(59004, 59005, 59006, 59007, 59008, 59009, 59010, 59011, 59012, 59013, 59014, 59015) c6( )c7(tJ, T, bM, jx, bO, Nt, aA, n, bS, gf, ct, ci, PJ, q, ad, eA, eJ, buD, dP, Hx, x, aE, ws, aT, h, aaw, bK, K, hK, he, ae, af, hZ, gj, Nr, aWm, Y, gl, lo, aF, po, g, Eg, PU, FX, cM, Z, iV, jb, FT, hq, Nq, ok, pZ, cA, abS, Ei, vL, pg, sv, pK, y, lB); #c1(FKBP6) c2(NP_001128683) c3(inactive peptidyl-prolyl cis-trans isomerase FKBP6 isoform b) c4(50503) c5(50504, 50505, 50506, 50507, 50508, 50509) c6( )c7(gU, alC, ND, aer, vH, PJ, af, azh, all); #c1(FKBPG) c2(NP_003593) c3(inactive peptidyl-prolyl cis-trans isomerase FKBPG isoform a) c4(139436) c5(139437, 139438, 139439, 139440, 139441, 139442) c6( )c7(gU, alC, ND, aer, vH, PJ, af, azh, all); #c1(FKBP7) c2(NP_001128684) c3 (peptidyl-prolyl cis-trans isomerase FKBP7 isoform b precursor) c4(50510) c5(50511, 50512, 50513, 50514, 50515, 50516) c6( )c7(PJ); #c1(FKBP7) c2(NP_851939) c3(peptidyl-prolyl cis-trans isomerase FKBP7 isoform a precursor) c4(254714, 254715) c5(254716, 254717, 254718, 254719, 254720, 254721, 254722, 254723, 254724, 254725, 254726, 254727) c6( )c7(PJ); #c1(FKBP8) c2(NP_036313) c3 (peptidyl-prolyl cis-trans isomerase FKBP8) c4(172959) c5(172960, 172961, 172962, 172963, 172964, 172965) c6( )c7(XW, PJ); #c1(FKBP9) c2(NP_001271270) c3(peptidyl-prolyl cis-trans isomerase FKBP9 isoform 2 precursor) c4(103799) c5(103800, 103801, 103802, 103803, 103804, 103805) c6( )c7(PJ); #c1(FKBP9) c2(NP_001271272) c3(peptidyl-prolyl cis-trans isomerase FKBP9 isoform 3) c4(103806) c5(103807, 103808, 103809, 103810, 103811, 103812) c6( )c7(PJ); #c1(FKBP9) c2(NP_009201) c3(peptidyl-prolyl cis-trans isomerase FKBP9 isoform 1 precursor) c4(171484, 171485) c5(171486, 171487, 171488, 171489, 171490, 171491, 171492, 171493, 171494, 171495, 171496, 171497) c6( )c7(PJ); #c1(FKBPL) c2(NP_071393) c3(FK506-binding protein-like) c4(211408) c5(211409, 211410, 211411, 211412, 211413, 211414, 211415, 211416, 211417, 211418, 211419, 211420, 211421) c6( )c7(T, gU, W, aW, eZ, aT); #c1(FKBPL) c2(XP_011545979) c3(FK506-binding protein-like isoform X1) c4(344808) c5(344809, 344810, 344811, 344812, 344813, 344814) c6( )c7(T, gU, W, aW, eZ, aT); #c1(FKRP) c2(XP_011525609) c3 (fukutin-related protein isoform X1) c4(320016) c5(320017, 320018, 320019, 320020, 320021, 320022, 320023, 320024, 320025, 320026, 320027) c6( )c7(buE, buG, nA, cd, azl, Ae, oj, Ak, K, Wq, buF, VC, aqZ, xs, FB, mx, km, cj, rx, bcZ, bD, lf); #c1(FKTN) c2(NP_001073270) c3 (fukutin isoform a) c4(35661) c5(35662, 35663, 35664, 35665, 35666, 35667, 35668) c6( )c7(T, buJ, nA, Mo, cc, als, Ae, hb, Ak, io, vc, buH, hB, Wq, VC, aqZ, xs, uY, axZ, mx, bul, km, bcZ, lf); #c1(FKTN) c2(NP_001185892) c3 (fukutin isoform b) c4(76664, 76665) c5(76666, 76667, 76668, 76669, 76670, 76671, 76672, 76673, 76674, 76675, 76676, 76677, 76678, 76679) c6( ) c7(T, buJ, nA, Mo, cc, als, Ae, hb, Ak, io, vc, buH, hB, Wq, VC, aqZ, xs, uY, axZ, mx, bul, km, bcZ, lf); #c1(FKTN) c2(XP_006717077) c3(fukutin isoform X12) c4(290690, 290691) c5(290692, 290693, 290694, 290695, 290696, 290697, 290698, 290699, 290700, 290701, 290702, 290703, 290704, 290705) c6( )c7(T, buJ, nA, Mo, cc, als, Ae, hb, Ak, io, vc, buH, hB, Wq, VC, aqZ, xs, uY, axZ, mx, bul, km, bcZ, lf); #c1(FKTN) c2(XP_011516681) c3(fukutin isoform X4) c4(310095) c5(310096, 310097, 310098, 310099, 310100, 310101, 310102) c6( )c7(T, buJ, nA, Mo, cc, als, Ae, hb, Ak, io, vc, buH, hB, Wq, VC, aqZ, xs, uY, axZ, mx, bul, km, bcZ, lf); #c1(FLAD1) c2(NP_001171820) c3(FAD synthase isoform 3) c4( )c5(71843, 71844, 71845, 71846, 71847) c6( )c7(bL, bM, om, e, M, wR, aj, j, H, J, L); #c1(FLAD1) c2(NP_001171821) c3(FAD synthase isoform 4) c4(71848, 71849) c5(71850, 71851, 71852, 71853, 71854, 71855, 71856, 71857, 71858, 71859, 71860, 71861, 71862, 71863, 71864, 71865, 71866, 71867) c6( )c7(bL, bM, om, e, M, wR, aj, j, H, J, L); #c1(FLAD1) c2(NP_079483) c3(FAD synthase isoform 1) c4(218284, 218285) c5(218286, 218287, 218288, 218289, 218290, 218291, 218292, 218293, 218294, 218295, 218296, 218297, 218298, 218299, 218300, 218301, 218302, 218303, 218304) c6( )c7(bL, bM, om, e, M, wR, aj, j, H, J, L); #c1 (FLAD1) c2(NP_958800) c3(FAD synthase isoform 2) c4(262205, 262206, 262207) c5( )c6( )c7(bL, bM, om, e, M, wR, aj, j, H, J, L); #c1 (FLAD1) c2(XP_005245559) c3(FAD synthase isoform X1) c4(266718) c5( )c6( )c7(bL, bM, om, e, M, wR, aj, j, H, J, L); #c1(FLAD1) c2(XP_005245560) c3(FAD synthase isoform X2) c4( )c5(266719, 266720, 266721, 266722, 266723, 266724) c6( )c7(bL, bM, om, e, M, wR, aj, j, H, J, L); #c1(FLCN) c2(NP_653207) c3(folliculin isoform 2) c4(239071) c5(239072, 239073, 239074, 239075, 239076, 239077, 239078, 239079, 239080, 239081, 239082, 239083, 239084, 239085, 239086, 239087, 239088, 239089, 239090, 239091, 239092, 239093) c6( )c7(T, Uc, WA, rd, aA, buk, buN, ci, fr, lu, q, lS, buM, hd, py, db, nX, bul, bj, K, lQ, ni, bug, r, aq, aF, cl, es, iA, bE, hk, buD, VB); #c1(FLCN) c2(NP_659434) c3(folliculin isoform 1) c4(239873, 239874) c5(239875, 239876, 239877, 239878, 239879, 239880) c6( ) c7(T, Uc, WA, rd, aA, buk, buN, ci, fr, lu, q, lS, buM, hd, py, db, nX, bul, bj, K, lQ, ni, bug, r, aq, aF, cl, es, iA, bE, hk, buO, VB); #c1(FLCN) c2(XP_011522022) c3 (folliculin isoform X3) c4(316709) c5( )c6( )c7(T, Uc, WA, rl, aA, buk, buN, ci, fr, lu, q, lS, buM, hd, py, db, nX, bul, bj, K, lQ, ni, bug, r, aq, af, cl, es, iA, bE, hk, buO, VB); #c1(FLG) c2(NP_002007) c3(filaggrin) c4(123202, 123203) c5(123204, 123205, 123206, 123207, 123208, 123209, 123210, 123211, 123212, 123213, 123214, 123215, 123216, 123217, 123218, 123219, 123220, 123221, 123222, 123223, 123224, 123225, 123226, 123227) c6( )c7(bM, bjX, T, JN, akc, buX, buP, btu, Pe, aj, pM, Jx, aVU, J, wD, NF, btD, aCF, oz, buR, apJ, buY, buW, aT, btA, fn, bam, lQ, XI, Cy, bvb, xP, buV, bull, aTq, ff, sp, fJ, ml, buZ, Sn, hQ, dN, cr, aEW, buS, fe, bul, Xk, en, eE, Vk, fq, aTi, dr, ck, cT, bva, OW, pa, buT, Jr, vZ, z, JM, NI, bvc, Km, y, cE, blT); #c1(FLG) c2(XP_011507631) c3 (filaggrin isoform X1) c4(297568, 297569) c5(297570, 297571, 297572, 297573, 297574, 297575, 297576, 297577) c6(297578, 297579) c7(bM, bjX, T, JN, akc, buX, buP, btu, Pe, aj, pM, Jx, aVU, J, wD, NF, btD, aCF, oz, buR, apJ, buY, buW, aT, btA, fn, bam, lQ, XI, Cy, bvb, xP, buV, bull, aTq, ff, sp, fJ, ml, buZ, Sn, hQ, dN, cr, aEW, buS, fe, bul, Xk, en, eE, Vk, fq, aTi, dr, ck, cT, bva, OW, pa, but, Jr, vZ, z, JM, NI, bvc, Km, y, cE, blT); #c1(FLG2) c2(NP_001014364) c3(filaggrin-2) c4(25817) c5(25818, 25819, 25820, 25821, 25822, 25823, 25824) c6( )c7(eE); #c1(FLG2) c2(XP_011507833) c3(filaggrin-2 isoform X1) c4( )c5(297980) c6( )c7(eE); #c1(FLI1) c2(NP_001257939) c3(Friend leukemia integration I transcription factor isoform 3) c4(95828) c5(95829, 95830, 95831, 95832, 95833, 95834) c6( )c7(T, dA, ya, zt, jh, bB, oH, ci, C, aGK, Wa, brK, uE, aY, mC, ad, hF, er, aHZ, bv, iJ, x, cq, ady, aCX, pX, avA, SV, aT, Ou, fb, ae, dx, jf, iF, lR, iU, aW, Y, oV, eH, eZ, eF, t, cm, fq, pb, adj, bw, ck, bnN, ac, bP, aDs, bE, bvd, hc, gX, dy, m, qF, fL, Xu, zu, bt); #c1(FLI1) c2(NP_001257941) c3(Friend leukemia integration I transcription factor isoform 4) c4(95835) c5(95836, 95837, 95838, 95839, 95840, 95841) c6( )c7(T, dA, ya, zt, jh, bB, oH, ci, C, aGK, Wa, brK, uE, aY, mC, ad, hF, er, aHZ, bv, iJ, x, cq, ady, aCX, pX, avA, SV, aT, Ou, fb, ae, dx, jf, iF, lR, il, aW, Y, oV, eH, eZ, eF, t, cm, fq, pb, adj, bw, ck, bnN, ac, bP, aDs, bE, bvd, hc, gX, dy, m, qF, fL, Xu, zu, bt); #c1(FLI1) c2(NP_002008) c3(Friend leukemia integration 1 transcription factor isoform 1) c4(123228, 123229) c5(123230, 123231, 123232, 123233, 123234, 123235, 123236, 123237) c6( )c7(T, dA, ya, zt, jh, bB, oH, ci, C, aGK, Wa, brK, uE, aY, mC, ad, hF, er, aHZ, bv, iJ, x, cq, ady, aCX, pX, avA, SV, aT, Ou, fb, ae, dx, jf, iF, lR, iU, aW, Y, oV, eH, eZ, eF, t, cm, fq, pb, adj, bw, ck, bnN, ac, bP, aDs, bE, bvd, hc, gX, dy, m, qF, fL, Xu, zu, bt); #c1(FLI1) c2(XP_011541003) c3(Friend leukemia integration I transcription factor isoform X1) c4( )c5(339535, 339536, 339537, 339538, 339539) c6( )c7(T, dA, ya, zt, jh, bB, OH, ci, C, aGK, Wa, brk, uE, aY, mC, ad, hF, er, aHZ, bv, iJ, x, cq, ady, aCX, pX, avA, SV, aT, Ou, fb, ae, dx, jf, iF, lR, iU, aW, Y, oV, eH, eZ, eF, t, cm, fq, pb, adj, bw, ck, bnN, ac, bP, aDs, bE, bvd, hc, gX, dy, m, qF, fL, Xu, zu, bt); #c1(FLI1) c2(NP_001243194) c3(protein flightless-1 homolog isoform 3) c4(90087) c5(90088, 90089, 90090, 90091, 90092, 90093) c6( )c7(T, dA, ya, zt, jh, aTy, OH, ci, uE, aGK, lR, aY, hF, XM, mC, ad, er, aHZ, iJ, bv, x, ady, cq, aCX, pX, avA, SV, aT, Ou, fb, ae, dx, jf, iF, il, aW, oV, eH, eZ, eF, t, cm, adj, pb, bw, ck, bnN, QG, bP, aDs, bvd, hc, gX, dy, m, qF, fL, Xu, zu, bt); #c1(FLI1) c2(NP_002009) c3(protein flightless-1 homolog isoform 1) c4(123238) c5(123239, 123240, 123241, 123242, 123243, 123244, 123245, 123246, 123247, 123248, 123249, 123250) c6( )c7(T, dA, ya, zt, jh, aTy, OH, ci, uE, aGK, lR, aY, hF, XM, mC, ad, er, aHZ, iJ, bv, x, ady, cq, aCX, pX, avA, SV, aT, Ou, fb, ae, dx, jf, iF, il, aW, oV, eH, eZ, eF, t, cm, adj, pb, bw, ck, bnN, QG, bP, aDs, bvd, hc, gX, dy, m, qF, fL, Xu, zu, bt); #c1(FLI1) c2(XP_005256612) c3(protein flightless-1 homolog isoform X1) c4(274231) c5( )c60) c7(T, dA, ya, zt, jh, aTy, oH, ci, uE, aGK, lR, aY, hF, XM, mC, ad, er, aHZ, iJ, bv, x, ady, cq, aCX, pX, avA, SV, aT, Ou, fb, ae, dx, jf, iF, il, aW, oV, eH, eZ, eF, t, cm, adj, pb, bw, ck, bnN, QG, bP, aDs, bvd, hc, gX, dy, m, qF, fL, Xu, zu, bt); #c1(FLNA) c2(NP_001104026) c3(filamin-A isoform 2) c4(42070) c5(42071, 42072, 42073, 42074, 42075, 42076, 42077, 42078, 42079, 42080, 42081, 42082) c6( )c7(T, dD, bvh, hl, fR, qo, lT, ea, acz, e, ha, aA, cz, n, aj, bS, j, bvf, ct, eq, bvn, Cd, Eh, afz, J, ad, hX, md, aE, asY, oj, aT, HD, dV, h, dL, ja, K, bvo, afx, rv, ae, aAe, cC, aa, bvm, eL, eG, A, ago, amx, bvg, up, cH, bvk, Y, aW, lW, hl, bvi, LI, aEW, aGd, aF, brr, Lk, aot, Z, akb, QK, bz, TA, WO, ac, iT, bve, Ji, uJ, Km, aS, Fw, bvj, fh, B, bvl): #c1 (FLNA) c2(NP_001447) c3(filamin-A isoform 1) c4(117891, 117892) c5(117893, 117894, 117895, 117896, 117897, 117898, 117899, 117900, 117901) c6( )c7(T, dD, bvh, hl, fR, qo, lT, ea, acz, e, ha, aA, cz, n, aj, bS, j, bvf, ct, eq, bvn, Cd, Eh, afz, J, ad, hX, md, aE, asY, oj, aT, HD, dV, h, dL, ja, K, bvo, afx, rv, ae, aAe, cC, aa, bvm, eL, eG, A, ago, amx, bvg, up, cH, bvk, Y, aW, lW, hl, bvi, LI, aEW, aGd, aF, brr, Lk, aot, Z, akb, QK, bz, TA, WO, ac, iT, bve, Ji, uJ, Km, aS, Fw, bvj, fh, B, bvl); #c1(FLNA) c2(XP_011529431) c3(filamin-A isoform X3) c4(324862) c5( )c6( ) c7(T, dD, bvh, hl, fR, qo, lT, ea, acz, e, ha, aA, cz, n, aj, bS, j, bvf, ct, eq, bvn, Cd, Eh, afz, J, ad, hX, md, aE, asY, oj, aT, hD, dV, h, dL, ja, K, bvo, afx, rv, ae, ale, cC, aa, bvm, eL, eG, A, ago, amx, bvg, up, cH, bvk, Y, aW, lW, hl, bvi, LI, aEW, aGd, aF, brr, Lk, aot, Z, akb, QK, bz, TA, WO, ac, iT, bve, Ji, uJ, Km, aS, Fw, bvj, fh, B, bvl); #c1(FLNA) c2(XP_011529432) c3(filamin-A isoform X4) c4( )c5(324863, 324864, 324865, 324866, 324867, 324868) c6( )c7(T, dD, bvh, hl, fR, qo, lT, ea, acz, e, ha, aA, cz, n, aj, bS, j, bvf, ct, eq, bvn, Cd, Eh, afz, J, ad, hX, md, aE, asY, oj, aT, hD, dV, h, dL, ja, K, bvo, afx, rv, ae, aAe, cC, aa, bvm, eL, eG, A, ago, amx, bvg, up, cH, bvk, Y, aW, lW, hl, bvi, L1, aEW, aGd, aF, brr, Lk, aot, Z, akb, QK, bz, TA, WO, ac, iT, bve, Ji, uJ, Km, aS, Fw, bvj, fh, B, bvl); #c1(FLNB) c2(NP_001157789) c3(filamin-B isoform 1) c4(62672) c5(62673, 62674, 62675, 62676, 62677, 62678, 62679, 62680, 62681, 62682, 62683, 62684) c6( )c7(oO, T, os, if, eb, bvq, cl, bvx, ci, aY, x, Na, azY, K, hu, hH, aa, dH, bbe, bhd, agj, eE, cm, bw, bvr, CC, iT, aiM, cj, k, qR, bvp, aw, fN, n, fr, qN, eW, br, h, eV, A, mR, bhg, aNd, bvv, pg, ut, it, B, bvt, jx, eX, rf, aA, bvu, aNe, V, o, jF, Nc, adV, Fg, rv, bA, iU, Y, afq, lo, iK, ck, BU, iM, ig, bvy, hr, fP, bt, bvw, kV, bvs, ow, qb, jh, oZ, hF, hX, av, F, oj, HD, il, yF, ul, je, KS, cr, eZ, aF, xH, hp, aot, iV, bz, Lj, ag, Fw, y, VK, hv); #c1(FLNB) c2(NP_001157790) c3(filamin-B isoform 3) c4(62685, 62686) c5(62687, 62688, 62689, 62690, 62691, 62692, 62693, 62694, 62695, 62696, 62697, 62698) c6( )c7(oO, T, os, if, eb, bvq, cl, bvx, ci, aY, x, Na, azY, K, hu, hH, aa, dH, bbe, bhd, agj, eE, cm, bw, bvr, CC, lT, aiM, cj, k, qR, bvp, aw, fN, n, fr, qN, eW, br, h, eV, A, mR, bhg, aNd, bvv, pg, ut, it, B, bvt, jx, eX, rf, aA, bvu, aNe, V, o, jF, Nc, adV, Fg, rv, bA, iU, Y, afq, lo, iK, ck, BU, iM, ig, bvy, hr, fP, bt, bvw, kV, bvs, ow, qb, jh, oZ, hF, hX, av, F, oj, HD, il, yF, ul, je, KS, cr, eZ, aF, xH, hp, aot, iV, bz, Lj, ag, Fw, y, VK, hv); #c1(FLNB) c2(NP_001157791) c3(filamin-B isoform 4) c4(62699) c5(62700, 62701, 62702, 62703, 62704, 62705) c6( ) c7(oO, T, os, if, eb, bvq, cl, bvx, ci, aY, x, Na, azY, K, hu, hH, aa, dH, bbe, bhd, agj, eE, cm, bw, bvr, CC, iT, aiM, cj, k, qR, bvp, aw, fN, n, fr, qN, eW, br, h, eV, A, mR, bhg, aNd, bvv, pg, ut, it, B, bvt, jx, eX, rf, aA, bvu, aNe, V, o, jF, Nc, adV, Fg, rv, bA, iU, Y, afq, lo, iK, ck, BU, iM, ig, bvy, hr, fP, bt, bvw, kV, bvs, ow, qb, jh, oZ, hF, hX, av, F, oj, HD, il, yF, ul, je, KS, cr, eZ, aF, xH, hp, aot, iV, bz, Lj, ag, Fw, y, VK, hv); #c1(FLNB) c2(NP_001448) c3(filamin-B isoform 2) c4(117902, 117903) c5(117904, 117905, 117906, 117907, 117908, 117909, 117910, 117911, 117912, 117913, 117914, 117915) c6( )c7(oO, T, os, if, eb, bvq, cl, bvx, ci, aY, x, Na, azY, K, hu, hH, aa, dH, bbe, bhd, agj, BE, cm, bw, bvr, CC, lT, aiM, cj, k, qR, bvp, aw, fN, n, fr, qN, eW, br, h, eV, A, mR, bhg, aNd, bwv, pg, ut, it, B, bvt, jx, eX, rf, aA, bvu, aNe, V, o, jF, Nc, adV, Fg, rv, bA, iU, Y, afq, lo, iK, ck, BU, iM, ig, bvy, hr, fP, bt, bvw, kV, bvs, ow, qb, jh, oZ, hF, hX, av, F, oj, hD, il, yF, ul, je, KS, cr, eZ, af, xH, hp, aot, iV, bz, Lj, ag, Fw, y, VK, hv); #c1(FLNB) c2(XP_005265034) c3(filamin-B isoform X1) c4(279403) c5( )c6( )c7(oO, T, os, if, eb, bvq, cl, bvx, ci, aY, x, Na, azY, K, hu, hH, aa, dH, bbe, bhd, agj, eE, cm, bw, bvr, CC, iT, aiM, cj, k, qR, bvp, aw, fN, n, fr, qN, eW, br, h, eV, A, mR, bhg, aNd, bvv, pg, ut, it, B, bvt, jx, eX, rf, aA, bvu, aNe, V, o, jF, Nc, adV, Fg, rv, bA, il, Y, afq, lo, iK, ck, BU, iM, ig, bvy, hr, fP, bt, bvw, kV, bvs, ow, qb, jh, oZ, hF, hX, av, F, oj, hD, il, yF, ul, je, KS, cr, eZ, aF, xH, hp, aot, iV, bz, Lj, ag, Fw, y, VK, hv); #c1(FLNB) c2(XP_005265038) c3(filamin-B isoform X3) c4(279404) c5(279405, 279406, 279407, 279408, 279409, 279410) c6( )c7(oO, T, os, if, eb, bvq, cl, bvx, ci, aY, x, Na, azY, K, hu, hH, aa, dH, bbe, bhd, agj, eE, cm, bw, bvr, CC, iT, aiM, cj, k, qR, bvp, aw, fN, n, fr, qN, eW, br, h, eV, A, mR, bhg, aNd, bvv, pg, ut, it, B, bvt, jx, eX, rf, aA, bvu, aNe, V, o, jF, Nc, adV, Fg, rv, bA, iU, Y, afq, lo, ik, ck, BU, iM, ig, bvy, hr, fP, bt, bvw, kV, bvs, ow, qb, jh, oZ, hF, hX, av, F, oj, hD, il, yF, ul, je, KS, cr, eZ, aF, xH, hp, aot, iV, bz, Lj, ag, Fw, y, VK, hv); #c1(FLNC) c2(NP_001120959) c3(filamin-C isoform b) c4(44989) c5(44990, 44991, 44992, 44993, 44994, 44995) c6( )c7(bM, T, no, pe, Ws, iW, bvA, xs, ll, oj, aA, km, mx, aF, Fw, bvz, vQ); #c1(FLNC) c2(NP_001449) c3(filamin-C isoform a) c4(117916) c5(117917, 117918, 117919, 117920, 117921, 117922) c6( )c7(bM, T, no, pe, Ws, iW, bvA, xs, ll, oj, aA, km, mx, af, Fw, bvz, vQ); #c1(FLOT1) c2(NP_005794) c3(flotillin-1) c4(159383, 159384, 159385, 159386, 159387, 159388, 159389, 159390, 159391, 159392, 159393) c5(159394, 159395, 159396, 159397, 159398, 159399, 159400, 159401, 159402, 159403, 159404, 159405, 159406, 159407, 159408, 159409, 159410, 159411, 159412, 159413, 159414, 159415, 159416, 159417, 159418, 159419, 159420, 159421, 159422, 159423, 159424, 159425, 159426, 159427, 159428, 159429, 159430, 159431, 159432, 159433, 159434, 159435, 159436, 159437, 159438, 159439, 159440, 159441, 159442, 159443, 159444, 159445, 159446, 159447, 159448, 159449, 159450, 159451, 159452, 159453, 159454, 159455, 159456, 159457, 159458, 159459, 159460, 159461, 159462, 159463, 159464, 159465, 159466, 159467, 159468, 159469, 159470, 159471, 159472, 159473, 159474, 159475, 159476, 159477, 159478, 159479, 159480, 159481, 159482, 159483, 159484, 159485, 159486, 159487, 159488, 159489, 159490, 159491, 159492, 159493, 159494, 159495, 159496, 159497, 159498, 159499, 159500, 159501, 159502, 159503, 159504, 159505, 159506, 159507) c6( )c7(T, eM, aW, eZ, aT, cm, H, V, af, L); #c1(FLOT1) c2(XP_005248838) c3(flotillin-1 isoform X2) c4(268637) c5(268638, 268639, 268640, 268641, 268642, 268643, 268644, 268645, 268646, 268647, 268648, 268649, 268650, 268651, 268652, 268653, 268654, 268655) c6( )c7(T, eM, aW, eZ, aT, cm, H, V, af, L); #c1(FLOT1) c2(XP_006715010) c3(flotillin-1 isoform X3) c4(288391, 288392, 288393, 288394, 288395, 288396, 288397, 288398, 288399, 288400) c5(288401, 288402, 288403, 288404, 288405, 288406, 288407, 288408, 288409, 288410, 288411, 288412, 288413, 288414, 288415, 288416, 288417, 288418, 288419, 288420, 288421, 288422, 288423, 288424, 288425, 288426, 288427, 288428, 288429, 288430, 288431, 288432, 288433, 288434, 288435, 288436, 288437, 288438, 288439, 288440, 288441, 288442, 288443, 288444, 288445, 288446, 288447, 288448, 288449, 288450, 288451, 288452, 288453, 288454, 288455, 288456, 288457, 288458, 288459, 288460, 288461, 288462, 288463, 288464, 288465, 288466, 288467, 288468, 288469, 288470, 288471, 288472, 288473, 288474, 288475, 288476, 288477, 288478, 288479, 288480, 288481, 288482, 288483, 288484, 288485, 288486, 288487, 288488, 288489, 288490, 288491, 288492, 288493, 288494, 288495, 288496, 288497, 288498, 288499, 288500, 288501, 288502, 288503, 288504, 288505, 288506, 288507, 288508, 288509, 288510, 288511, 288512, 288513, 288514) c6( )c7(T, eM, aW, eZ, aT, cm, H, V, af, L); #c1(FLOT2) c2(NP_004466) c3(flotillin-2) c4(146579, 146580) c5(146581, 146582, 146583, 146584, 146585, 146586, 146587, 146588, 146589, 146590, 146591, 146592) c6( )c7(fU, T, dA, eM, aW, Y, oW, aT, aot, ci, K, bmA, iK, cp); #c1(FLRT2) c2(NP_037363) c3(leucine-rich repeat transmembrane protein FLRT2 precursor) c4(175012, 175013) c5(175014, 175015, 175016, 175017, 175018, 175019, 175020, 175021, 175022, 175023, 175024, 175025, 175026, 175027, 175028, 175029) c6( )c7(y, eZ); #c1(FLRT3) c2(NP_938205) c3(leucine-rich repeat transmembrane protein FLRT3 precursor) c4(259790, 259791) c5(259792, 259793, 259794, 259795, 259796, 259797, 259798, 259799, 259800, 259801, 259802, 259803, 259804, 259805, 259806, 259807) c6( )c7(bvB, UF); #c1(FLT1) c2(NP_001153392) c3(vascular endothelial growth factor receptor 1 isoform 2 precursor) c4(60565) c5(60566, 60567) c6( )c7(T, aC, UH, pf, ea, e, al, eb, bB, fd, ci, aY, Hr, x, Vp, bv, aE, rw, K, hu, eL, DA, eG, JI, aW, bvD, Ls, NR, g, as, eF, uC, cm, DA, bw, qJ, y, eM, nB, iT, vy, cj, k, m, bvC, cE, YK, Vj, cZ, dD, iB, aw, ht, NT, n, fr, eh, ai, br, b, jz, gd, dL, Jn, dd, Um, cY, sp, pm, fs, W, bP, dB, hk, Vy, pg, aAl, B, aJJ, fM, dA, AL, jx, aA, cz, wo, v, j, AD, q, eA, MP, aT, cy, Tj, jj, sR, rv, ae, dl, vT, oV, Y, r, en, bu, t, acp, pb, ck, nM, ac, gX, dy, im, bt, VB, hS, sY, hl, iW, Hn, Fb, ah, eq, hF, Fc, ad, afb, Jm, fU, av, iC, ib, NS, Hk, vB, nJ, ul, d, qt, ff, eH, cr, eZ, aFr, aF, iV, Hp, LB, gk, Ew, pc, nb, bE, i, lf, vt, X, jr); #c1(FLT1) c2(NP_001153502) c3(vascular endothelial growth factor receptor 1 isoform 3 precursor) c4(60604) c5(60605, 60606, 60607, 60608, 60609, 60610, 60611) c6( )c7(T, aC, UH, pf, ea, e, al, eb, bB, fd, ci, aY, Hr, x, Vp, bv, aE, rw, K, hu, eL, DA, eG, JI, aW, bvD, Ls, NR, g, as, eF, uC, cm, QA, bw, qJ, y, eM, nB, iT, vy, cj, k, m, bvC, cE, YK, Vj, cZ, dD, iB, aw, ht, NT, n, fr, eh, ai, br, b, jz, gd, dl, Jn, dd, Um, cY, sp, pm, fs, W, bP, dB, hk, Vy, pg, aAl, B, aJJ, fM, dA, AL, jx, aA, cz, wo, v, j, A, q, eA, MP, aT, cy, Tj, jj, sR, rv, ae, dl, vT, oV, Y, r, en, bu, t, acp, pb, ck, nM, ac, gX, dy, im, bt, VB, hS, sY, hl, iW, Hn, Fb, ah, eq, hF, Fc, ad, afb, Jm, fU, av, iC, ib, NS, Hk, vB, nJ, ul, d, qt, ff, eH, cr, eZ, afr, aF, iV, Hp, LB, gk, Ew, pc, nb, bE, i, lf, vt, X, jr); #c1(FLT1) c2(NP_001153503) c3(vascular endothelial growth factor receptor 1 isoform 4 precursor) c4(60612, 60613) c5(60614, 60615, 60616, 60617, 60618, 60619, 60620, 60621, 60622, 60623, 60624, 60625, 60626, 60627, 60628, 60629) c6( )c7(T, aC, UH, pf, ea, e, al, eb, bB, fd, ci, aY, Hr, x, Vp, bv, aE, rw, K, hu, eL, DA, eG, JI, aW, bvD, Ls, NR, g, as, eF, uC, cm, QA, bw, qJ, y, eM, nB, iT, vy, cj, k, m, bvC, cE, YK, Vj, cZ, dD, iB, aw, ht, NT, n, fr, eh, ai, br, b, jz, gd, dL, Jn, dd, Um, cY, sp, pm, fs, W, bP, dB, hk, Vy, pg, aAl, B, aJJ, fM, dA, AL, jx, aA, cz, wo, v, j, AD, q, eA, MP, aT, cy, Tj, jj, sR, rv, ae, dl, vT, oV, Y, r, en, bu, t, acp, pb, ck, nM, ac, gX, dy, im, bt, VB, hS, sY, hl, iW, Hn, Fb, ah, eq, hF, Fc, ad, afb, Jm, fU, av, iC, ib, NS, Hk, vB, nJ, ul, d, qt, ff, eH, cr, eZ, aFr, aF, iV, Hp, LB, gk, Ew, pc, nb, bE, i, lf, vt, X, jr); #c1(FLT1) c2(NP_002010) c3(vascular endothelial growth factor receptor 1 isoform 1 precursor) c4(123251, 123252, 123253) c5(123254, 123255, 123256, 123257, 123258, 123259, 123260, 123261, 123262, 123263, 123264, 123265, 123266, 123267, 123268, 123269, 123270, 123271) c6( )c7(T, aC, UH, pf, ea, e, al, eb, bB, fd, ci, aY, Hr, x, Vp, bv, aE, rw, K, hu, eL, DA, eG, JI, aW, bvD, Ls, NR, g, as, eF, uC, cm, QA, bw, qJ, y, eM, nB, iT, vy, cj, k, m, bvC, cE, YK, Vj, cZ, dD, iB, aw, ht, NT, n, fr, eh, ai, br, b, jz, gd, dL, Jn, dd, Um, cY, sp, pm, fs, W, bP, dB, hk, Vy, pg, aAl, B, aJJ, fM, dA, AL, jx, aA, cz, wo, v, j, AD, q, eA, MP, aT, cy, Tj, jj, sR, rv, ae, dl, vT, oV, Y, r, en, bu, t, acp, pb, ck, nM, ac, gX, dy, im, bt, VB, hS, sY, hl, iW, Hn, Fb, ah, eq, hF, Fc, ad, afb, Jm, fU, av, iC, ib, NS, Hk, vB, nJ, ul, d, qt, ff, eH, cr, eZ, aFr, aF, iV, Hp, LB, gk, Ew, pc, nb, bE, i, lf, vt, X, jr); #c1(FLT3) c2(NP_004110) c3(receptor-type tyrosine-protein kinase FLT3 precursor) c4(143641, 143642, 143643) c5(143644, 143645, 143646, 143647, 143648, 143649, 143650, 143651, 143652, 143653, 143654, 143655, 143656, 143657, 143658, 143659, 143660, 143661) c6( )c7(eW, aiu, T, aC, ya, qx, jh, bs, n, axk, bvH, OH, aj, v, oY, hF, bvE, q, br, J, SW, zn, ai, er, bvG, fX, hd, aE, x, dn, aT, dz, hn, Ou, K, lQ, jj, iZ, pq, il, aBl, dx, bvF, bA, iF, jf, Nr, d, il, hG, bvl, oV, et, bvJ, fO, gK, ace, r, po, gm, pm, hf, Fu, f, cM, cm, At, bw, yR, Nq, ov, ou, ck, iT, dB, kz, cj, k, eA, ph, bt, kS); #c1(FLT3LG) c2(NP_001450) c3(fms-related tyrosine kinase 3 ligand isoform 1 precursor) c4(117923) c5(117924, 117925, 117926, 117927, 117928, 117929, 117930, 117931, 117932, 117933, 117934, 117935) c6(117936, 117937) c7(oO, bM, T, FR, ya, iW, fd, axk, @p, aYs, ai, q, ad, eA, eW, jZ, aT, dz, Ou, pq, ae, d, je, aW, r, t, cm, bw, ck, iM, dB, Lj, cj, oX, y, bt, bvK); #c1(FLT3LG) c2(XP_006723179) c3(fms-related tyrosine kinase 3 ligand isoform X8) c4( )c5(295021) c6( )c7(oO, bM, T, fR, ya, iW, fd, axk, [p, aYs, ai, q, ad, eA, eW, jZ, aT, dz, Ou, pq, ae, d, je, aW, r, t, cm, bw, ck, iM, dB, Lj, cj, oX, y, bt, bvK); #c1 (FLT3LG) c2(XP_011524979) c3(fms-related tyrosine kinase 3 ligand isoform X9) c4(319464) c5(319465, 319466, 319467, 319468, 319469, 319470, 319471, 319472) c6(319473, 319474) c7(oO, bM, T, fR, ya, iW, fd, axk, Qp, aYs, ai, q, ad, eA, eW, jZ, aT, dz, Ou, pq, ae, d, je, aW, r, t, cm, bw, ck, iM, dB, Lj, cj, oX, y, bt, bvK); #c1(FLT3LG) c2(XP_011524981) c3(fms-related tyrosine kinase 3 ligand isoform X10) c4( ) c5(319475) c6( )c7(oO, bM, T, fR, ya, iW, fd, axk, Qp, aYs, ai, q, ad, eA, eW, jZ, aT, dz, Ou, pq, ae, d, je, aW, r, t, cm, bw, ck, iM, dB, Lj, cj, oX, y, bt, bvk); #c1(FLT4) c2(NP_002011) c3(vascular endothelial growth factor receptor 3 isoform 2 precursor) c4(123272, 123273) c5(123274, 123275, 123276, 123277, 123278, 123279) c6( )c7(T, uk, aC, Tb, ea, if, hE, fd, bB, ci, aGm, aE, bll, Vv, K, aCU, cp, Jl, aW, g, eE, eF, uC, cm, eM, nB, xD, go, vy, cj, PO, k, m, ym, cE, dD, QO, sw, fr, qN, Cd, jz, boz, aq, yq, W, Vy, B, fM, aA, WD, Cb, q, nv, aT, ae, af, kl, s, xR, Y, r, cl, t, ck, bvL, aor, PN, bt, sY, jh, eq, ad, hX, Jm, av, aop, ib, db, Ot, tT, bvM, MG, azH, d, cr, aF, iR, bz, pu, bE, y, EB); #c1(FLT4) c2(NP_891555) c3(vascular endothelial growth factor receptor 3 isoform 1 precursor) c4(257317) c5(257318, 257319, 257320, 257321, 257322, 257323, 257324, 257325, 257326, 257327, 257328, 257329, 257330, 257331, 257332, 257333, 257334, 257335) c6( )c7(T, uk, aC, Tb, ea, if, hE, fd, bB, ci, aGm, aE, bll, Vv, K, aCU, cp, JI, aW, g, eE, eF, uC, cm, eM, nB, xD, go, vy, cj, PO, k, m, ym, cE, dD, QO, sw, fr, qN, Cd, jz, boz, aq, yq, W, Vy, B, fM, aA, WD, Cb, q, nv, aT, ae, af, kl, s, xR, Y, r, cl, t, ck, bvl, aor, PN, bt, sY, jh, eq, ad, hX, Jm, av, aop, ib, db, Ot, tT, bvM, MG, azH, d, cr, aF, iR, bz, pu, bE, y, EB); #c1(FLT4) c2(XP_011532785) c3(vascular endothelial growth factor receptor 3 isoform X6) c4(328865) c5( )c6( )c7(T, uk, aC, Tb, ea, if, hE, fd, bB, ci, aGm, aE, bll, Vv, K, aCU, cp, JI, aW, g, eE, eF, uC, cm, eM, nB, xD, go, vy, cj, PO, k, m, ym, cE, dD, QO, sw, fr, qN, Cd, jz, boz, aq, yq, W, Vy, B, fM, aA, WD, Cb, q, nv, aT, ae, af, kl, s, xR, Y, r, cl, t, ck, bvL, aor, PN, bt, sY, jh, eq, ad, hX, Jm, av, aop, ib, db, Ot, tT, bvM, MG, azH, d, cr, aF, iR, bz, pu, bE, y, EB); #c1(FLVCR1) c2(NP_054772) c3(feline leukemia virus subgroup C receptor-related protein 1) c4(177725) c5(177726, 177727, 177728, 177729, 177730, 177731) c6( )c7(T, FR, Pt, oe, zo, eA, bvN, ad, CJ, aE, bvS, ae, bvO, bvR, are, cr, xw, nC, bvP, bvl, U, Br, aS); #c1(FLVCR2) c2(NP_001182212) c3(feline leukemia virus subgroup C receptor-related protein 2 isoform 2) c4(74687) c5(74688, 74689, 74690, 74691, 74692, 74693) c6( )c7(bM, aoU, bvT, al, Wb, VV, l, u, aMC, ZH, s, CN, FD, Tp, Pd, bvP, ej, bF, qe, z, aS, ER); #c1(FLVCR2) c2(NP_060261) c3 (feline leukemia virus subgroup C receptor-related protein 2 isoform 1) c4(195109) c5(195110, 195111, 195112, 195113, 195114, 195115) c6( )c7(bM, aol, bvT, al, Wb, VV, l, u, aMC, ZH, s, CN, FD, Tp, Pd, bvP, ej, bF, qe, z, aS, ER); #c1(FMN1) c2(NP_001096654) c3(formin-1 isoform b) c4(41263, 41264) c5(41265, 41266, 41267, 41268, 41269, 41270, 41271, 41272, 41273, 41274, 41275, 41276) c6( )c7(arB, dY, bvV, r, iG, q, cm, bvU, ad); #c1(FMN1) c2(NP_001264242) c3(formin-1 isoform a) c4(97640) c5(97641, 97642, 97643, 97644, 97645, 97646) c6( )c7(arB, dY, bvV, r, iG, q, cm, bvU, ad); #c1(FMN1) c2(NP_001264243) c3(formin-1 isoform c) c4(97647) c5(97648, 97649, 97650, 97651, 97652, 97653) c6( )c7(arB, dY, bvV, r, iG, q, cm, bvU, ad); #c1(FMN2) c2(NP_064450) c3(formin-2) c4(202527, 202528) c5(202529, 202530, 202531, 202532, 202533, 202534, 202535, 202536, 202537, 202538, 202539, 202540, 202541, 202542, 202543, 202544, 202545, 202546, 202547, 202548, 202549, 202550, 202551, 202552, 202553) c6( )c7(ou, A, ac, c, e, hn, j, fr, V, fq); #c1(FMN2) c2(XP_011542539) c3(formin-2 isoform X2) c4( )c5(341088) c6( )c7(ou, A, ac, c, e, hn, j, fr, V, fq); #c1(FMNL1) c2(NP_005883) c3(formin-like protein 1) c4(160099) c5(160100, 160101, 160102, 160103, 160104, 160105, 160106, 160107, 160108, 160109, 160110, 160111) c6( )c7(ck, T, iW, jh, cj, hf); #c1(FMNL1) c2(XP_006722129) c3 (formin-like protein 1 isoform X7) c4( )c5(294627) c6( )c7(ck, T, iW, jh, cj, hf); #c1(FMNL1) c2(XP_006722132) c3(formin-like protein 1 isoform X9) c4( )c5(294628, 294629, 294630, 294631, 294632) c6( )c7(ck, T, iW, jh, cj, hf); #c1(FMNL1) c2(XP_006722133) c3(formin-like protein 1 isoform X10) c4(294633) c5( )c6( )c7(ck, T, iW, jh, cj, hf); #c1(FMNL2) c2(NP_443137) c3(formin-like protein 2) c4(229612) c5(229613, 229614, 229615, 229616, 229617, 229618) c6( )c7(T, oe, r, q, B, bN); #c1(FMNL2) c2(XP_005246320) c3(formin-like protein 2 isoform X5) c4(267044) c5(267045, 267046, 267047, 267048, 267049, 267050) c6( ) c7(T, oe, r, q, B, bN); #c1(FMNL3) c2(NP_783863) c3(formin-like protein 3 isoform 1) c4(251771) c5(251772, 251773, 251774, 251775, 251776, 251777) c6( )c7(dl, cc, A, mx); #c1(FMNL3) c2(NP_944489) c3(formin-like protein 3 isoform 2) c4(260615) c5(260616, 260617, 260618, 260619, 260620, 260621) c6( )c7(dQ, cc, A, mx); #c1(FMNL3) c2(XP_005269275) c3(formin-like protein 3 isoform X4) c4(282520, 282521) c5(282522, 282523, 282524, 282525, 282526, 282527, 282528, 282529, 282530, 282531, 282532, 282533) c6( ) c7(dl, cc, A, mx); #c1(FMNL3) c2(XP_011537275) c3(formin-like protein 3 isoform X7) c4(334998) c5( )c6( )c7(dl, cc, A, mx); #c1(FM01) c2(NP_001269621) c3(dimethylaniline monooxygenase [N-oxide-forming] 1 isoform a) c4(102220) c5(102221, 102222, 102223) c6( )c7(fQ, bK, Vd, c); #c1(FM01) c2(NP_001269622) c3(dimethylaniline monooxygenase [N-oxide-forming] 1 isoform b) c4( )c5(102224, 102225, 102226, 102227, 102228, 102229, 102230, 102231) c6( )c7(fQ, bk, Vd, c); #c1(FM01) c2(NP_001269623) c3(dimethylaniline monooxygenase [N-oxide-forming] 1 isoform c) c4(102232) c5(102233, 102234, 102235, 102236, 102237, 102238) c6( )c7(fQ, bk, Vd, c); #c1(FM01) c2(XP_005245095) c3(dimethylaniline monooxygenase [N-oxide-forming] 1 isoform X4) c4(266275) c5(266276, 266277, 266278, 266279, 266280) c6( )c7(fQ, bK, Vd, c); #c1(FM02) c2(NP_001451) c3(dimethylaniline monooxygenase [N-oxide-forming] 2 isoform a) c4(1/7938) c5(117939, 117940, 117941, 117942, 117943, 117944, 117945, 117946, 117947, 117948) c6( )c7(dP, iT, bX, qt); #c1(FM03) c2(NP_008825) c3(dimethylaniline monooxygenase [N-oxide-forming] 3) c4(168840) c5(168841, 168842, 168843, 168844, 168845, 168846, 168847, 168848, 168849, 168850) c6( )c7(bF, T, dD, c, s, iT, ea, r, cz, dq, eq, K, bX, EZ, bz, q, dr); #c1(FM03) c2(XP_005245101) c3(dimethylaniline monooxygenase [N-oxide-forming] 3 isoform X2) c4(266281) c5(266282, 266283, 266284, 266285, 266286, 266287) c6( )c7(bF, T, dD, c, s, iT, ea, r, cz, dq, eq, K, bX, EZ, bz, q, dr); #c1(FM03) c2(XP_011507648) c3(dimethylaniline monooxygenase [N-oxide-forming] 3 isoform X1) c4(297594) c5(297595, 297596, 297597, 297598, 297599, 297600) c6( )c7(bF, T, dD, c, s, iT, ea, r, cz, dq, eq, K, bX, EZ, bz, q, dr); #c1(FM04) c2(NP_002013) c3(dimethylaniline monooxygenase [N-oxide-forming] 4) c4(123280, 123281) c5(123282, 123283, 123284, 123285, 123286, 123287) c6( )c7(qt); #c1(FM04) c2(XP_005245105) c3 (dimethylaniline monooxygenase [N-oxide-forming] 4 isoform X6) c4( )c5(266288) c6( )c7(qt); #c1(FMOD) c2(NP_002014) c3(fibromodulin precursor) c4(123288, 123289, 123290) c5(123291, 123292, 123293, 123294, 123295, 123296, 123297, 123298, 123299, 123300, 123301, 123302, 123303, 123304, 123305, 123306, 123307, 123308) c6( )c7(gk, dD, W, pG, ea, al, Be, cj, lj, cz, dy, eq); #c1(FMRI) c2(NP_001172004) c3(fragile X mental retardation protein 1 isoform IS06) c4(72180) c5(72181, 72182, 72183, 72184, 72185, 72186) c6( )c7(bM, bdo, T, ahc, aku, bO, Jd, aTS, aR, Gt, wM, il, K, asP, aW, Az, hl, FS, Ku, JI, aiv, wT, Kv, Kr, U, yA, Uw, Kk, To, bvW, ct, nA, rc, aU, b, i, h, UD, yZ, he, on, alM, aml, agG, atf, zZ, hq, ky, cA, agZ, pQ, GC, Kh, pK, V, Kx, boY, KB, QU, aT, wP, bK, an, rz, afx, bvX, kl, abi, aeS, f, xw, bN, alT, Ki, rR, kP, c, kB, aan, UF, gT, aen, Fl, qX, aZ, ha, bS, uu, bwa, bfD, xt, av, bvY, vJ, nd, Kq, Ng, nf, dq, Z, Kp, KI, dr, dR, bwb, bC, bvZ, xg, Ex); #c1(FMRI) c2(NP_001172005) c3(fragile X mental retardation protein 1 isoform IS07) c4(72187, 72188, 72189, 72190) c5(72191, 72192, 72193, 72194, 72195, 72196, 72197, 72198, 72199, 72200, 72201, 72202, 72203, 72204, 72205, 72206, 72207, 72208, 72209, 72210, 72211, 72212, 72213, 72214) c6( )c7(bM, bdo, T, ahc, aKu, bO, Jd, aTS, aR, Gt, wM, il, K, asP, aW, Az, hl, FS, Ku, JI, aiv, wT, Kv, Kr, U, yA, Uw, Kk, To, bvW, ct, nA, rc, aU, b, i, h, UD, yZ, he, on, alM, aml, agG, atf, zZ, hq, ky, cA, agZ, pQ, GC, Kh, pK, V, Kx, boY, KB, QU, aT, wP, bK, an, rz, afx, bvX, kl, abi, aeS, f, xw, bN, alT, Ki, rR, kP, c, kB, aan, UF, gT, aen, Fl, qX, aZ, ha, bS, uu, bwa, bfD, xt, av, bvY, vJ, nd, Kq, Ng, nf, dq, Z, Kp, KI, dr, dR, bwb, bC, bvZ, xg, Ex); #c1(FMRI) c2(NP_001172010) c3(fragile X mental retardation protein 1 isoform IS012) c4( )c5(72226, 72227, 72228, 72229, 72230, 72231) c6( ) c7(bM, bdo, T, ahc, aku, bO, Jd, aTS, aR, Gt, wM, il, K, asP, aW, Az, hl, FS, Ku, JI, aiv, wT, Kv, Kr, U, yA, Uw, Kk, To, bvW, ct, nA, rc, aU, b, i, h, UD, yZ, he, on, alM, aml, agG, atf, zZ, hq, ky, cA, agZ, pQ, GC, Kh, pK, V, Kx, boY, KB, QU, aT, wP, bK, an, rz, afx, bvX, kl, abi, aeS, f, xw, bN, alT, Ki, rR, kP, c, kB, aan, UF, gT, aen, Fl, qX, aZ, ha, bS, uu, bwa, bfD, xt, av, bvY, vJ, nd, Kq, Ng, nf, dq, Z, Kp, KI, dr, dR, bwb, bC, bvZ, xg, Ex); #c1(FMRI) c2(NP_001172011) c3(fragile X mental retardation protein 1 isoform IS09) c4( )c5(72232, 72233, 72234, 72235) c6( )c7(bM, bdo, T, ahc, aKu, bO, Jd, aTS, aR, Gt, wM, il, K, asP, aW, Az, hl, FS, Ku, JI, aiv, wT, Kv, Kr, U, yA, Uw, Kk, To, bvW, ct, nA, rc, aU, b, i, h, UD, yZ, he, on, alM, aml, agG, atf, zZ, hq, ky, cA, agZ, pQ, GC, Kh, pk, V, Kx, boY, KB, QU, aT, wP, bK, an, rz, afx, bvX, kl, abi, aeS, f, xw, bN, alT, Ki, rR, kP, c, kB, aan, UF, gT, aen, Fl, qX, aZ, ha, bS, uu, bwa, bfD, xt, av, bvY, vJ, nd, Kq, Ng, nf, dq, Z, Kp, KI, dr, dR, bwb, bC, bvZ, xg, Ex); #c1(FMRI) c2(NP_002015) c3(fragile X mental retardation protein 1 isoform IS01) c4(123309, 123310, 123311) c5(123312, 123313, 123314, 123315, 123316, 123317, 123318, 123319, 123320, 123321, 123322, 123323) c6( )c7(bM, bdo, T, ahc, aku, bO, Jd, aTS, aR, Gt, wM, il, K, asP, aW, Az, hl, FS, Ku, JI, aiv, wT, Kv, Kr, U, yA, Uw, Kk, To, bvW, ct, nA, rc, aU, b, i, h, UD, yZ, he, on, alM, aml, agG, atf, zZ, hq, ky, cA, agZ, pQ, GC, Kh, pK, V, Kx, boY, KB, QU, aT, wP, bK, an, rz, afx, bvX, kl, abi, aeS, f, xw, bN, alT, Ki, rR, kP, c, kB, aan, UF, gT, aen, Fl, qX, aZ, ha, bS, uu, bwa, bfD, xt, av, bvY, vJ, nd, Kq, Ng, nf, dq, Z, Kp, KI, dr, dR, bwb, bC, bvZ, xg, Ex); #c1(FN1) c2(NP_002017) c3(fibronectin isoform 3 preproprotein) c4(123334) c5(123335, 123336, 123337, 123338, 123339, 123340, 123341, 123342) c6(123343, 123344) c7(bM, T, os, ea, if, al, fd, bB, aTy, zh, ci, lR, Lw, aao, x, Na, aE, ws, rJ, aeO, us, azm, K, eL, bc, cp, aa, DA, eG, zQ, aeR, yo, aW, yK, hl, g, as, BE, eF, bwe, uC, cm, aAW, ba, lt, eM, nB, iA, iT, go, vA, mx, vy, cj, Gs, Zn, uJ, qr, wx, Hb, m, fy, Vj, iu, bcO, bks, rt, cZ, dD, sw, aw, n, aj, fr, apt, uE, qN, zP, cd, asS, ai, yn, eW, b, ft, Xe, kl, h, nm, By, dL, ey, dd, vx, Ha, bwj, Nr, A, bwi, fJ, hQ, bwc, aq, po, kp, mo, dw, mb, cM, wU, op, bwd, By, ky, lX, dB, xO, Fi, bjd, ajd, JM, at, E, uz, XG, pg, bwg, it, B, arO, vQ, bL, fM, dA, atB, dk, fG, jx, aA, cz, v, j, C, sl, yV, AD, Eh, q, J, bag, er, eA, lO, qK, aT, cy, dz, Nc, Fx, zp, bq, lM, ae, bsq, af, dl, Lu, wr, s, il, Ue, Y, oV, bg, Pg, ev, r, aYp, aSJ, en, hf, apx, t, Vq, iK, pb, wA, vk, amF, ck, sS, nM, iM, vZ, WP, hc, nG, DW, hr, adR, bwh, dy, fD, bt, rA, VB, vi, sY, id, qb, LX, ha, jh, vm, dG, oZ, eq, ad, aoH, bwf, aHF, dP, hd, av, db, Os, vw, aSf, hP, ji, vP, uX, d, lR, je, bwk, qt, ff, xs, wc, eH, cr, dN, hv, aFr, aF, si, VI, aiz, eZ, iR, bz, dr, Hp, Nq, LB, gk, es, mi, zr, bE, Hs, aS, bC, be, i, y, X, jr); #c1(FN1) c2(NP_473375) c3(fibronectin isoform 7 preproprotein) c4(230500) c5(230501, 230502, 230503, 230504, 230505, 230506, 230507, 230508) c6(230509, 230510) c7(bM, T, os, ea, if, al, fd, bB, aTy, zh, ci, lR, Lw, aao, x, Na, aE, ws, rJ, ael, us, azm, K, eL, bc, cp, aa, DA, eG, zQ, aeR, yo, aW, yK, hl, g, as, BE, eF, bwe, uC, cm, aAW, ba, lt, eM, nB, iA, iT, go, vA, mx, vy, cj, Gs, Zn, uJ, qr, wx, Hb, m, fy, Vj, iu, bcO, bks, rt, cZ, dD, sw, aw, n, aj, fr, apt, uE, qN, zP, cd, asS, ai, yn, eW, b, ft, Xe, kl, h, nm, By, dL, ey, dd, vx, Ha, bwj, Nr, A, bwi, fJ, hQ, bwc, aq, po, kp, mo, dw, mb, cM, wU, op, bwd, Qy, ky, lX, dB, xO, Fi, bjd, ajd, JM, at, E, uz, XG, pg, bwg, it, B, arO, vQ, bL, fM, dA, atB, dk, fG, jx, aA, cz, v, j, C, sl, yV, AD, Eh, q, J, bag, er, eA, lO, qK, aT, cy, dz, Nc, Fx, zp, bq, lM, ae, bsq, af, dl, Lu, wr, s, iU, Ue, Y, oV, bg, Pg, ev, r, aYp, aSJ, en, hf, apx, t, Vq, iK, pb, wA, vk, amF, ck, sS, nM, iM, vZ, WP, hc, nG, DW, hr, adR, bwh, dy, fD, bt, rA, VB, vi, sY, id, qb, LX, ha, jh, vm, dG, oZ, eq, ad, aoH, bwf, aHF, dP, hd, av, db, Os, vw, aSf, hP, ji, vP, uX, d, lR, je, bwk, qt, ff, xs, wc, eH, cr, dN, hv, aFr, aF, si, VI, aiz, eZ, iR, bz, dr, Hp, Nq, LB, gk, es, mi, zr, bE, Hs, aS, bC, be, i, y, X, jr); #c1(FN1) c2(NP_997639) c3(fibronectin isoform 6 preproprotein) c4(265624) c5(265625, 265626, 265627, 265628, 265629, 265630, 265631, 265632) c6(265633, 265634) c7(bM, T, os, ea, if, al, fd, bB, aTy, zh, ci, lR, Lw, aao, x, Na, aE, ws, rJ, aeO, us, azm, K, eL, bc, cp, aa, DA, eG, zQ, aeR, yo, aW, yK, hl, g, as, eE, eF, bwe, uC, cm, aAW, ba, lt, eM, nB, iA, iT, go, vA, mx, vy, cj, Gs, Zn, uJ, qr, wx, Hb, m, fy, Vj, iu, bcO, bks, rt, cZ, dD, sw, aw, n, aj, fr, apt, uE, qN, zP, cd, asS, ai, yn, eW, b, ft, Xe, kl, h, nm, By, dL, ey, dd, vx, Ha, bwj, Nr, A, bwi, fJ, hQ, bwc, aq, po, kp, mo, dw, mb, cM, wU, op, bwd, By, ky, lX, dB, xO, Fi, bjd, ajd, JM, at, E, uz, XG, pg, bwg, it, B, arO, vQ, bL, fM, dA, atB, dk, fG, jx, aA, cz, v, j, C, sl, yV, AD, Eh, q, J, bag, er, eA, lO, qK, aT, cy, dz, Nc, Fx, zp, bq, IM, ae, bsq, af, dl, Lu, wr, s, il, Ue, Y, oV, bg, Pg, ev, r, aYp, aSJ, en, hf, apx, t, Vq, iK, pb, wA, vk, amF, ck, sS, nM, iM, vZ, WP, hc, nG, DW, hr, adR, bwh, dy, fD, bt, rA, VB, vi, sY, id, qb, LX, ha, jh, vm, dG, oZ, eq, ad, aoH, bwf, aHF, dP, hd, av, db, Os, vw, aSf, hP, ji, vP, uX, d, lR, je, bwk, qt, ff, xs, wc, eH, cr, dN, hv, aFr, aF, si, VI, aiz, eZ, iR, bz, dr, Hp, Nq, LB, gk, es, mi, zr, bE, Hs, aS, bC, be, i, y, X, jr); #c1(FN1) c2(NP_997641) c3(fibronectin isoform 5 preproprotein) c4(265635) c5(265636, 265637, 265638, 265639, 265640, 265641, 265642, 265643) c6(265644, 265645) c7(bM, T, os, ea, if, al, fd, bB, aTy, zh, ci, lR, Lw, aao, x, Na, aE, ws, rJ, aeO, us, azm, K, eL, bc, cp, aa, DA, eG, zQ, aeR, yo, aW, yK, hl, g, as, eE, eF, bwe, uC, cm, aAW, ba, lt, eM, nB, iA, iT, go, vA, mx, vy, cj, Gs, Zn, uJ, qr, wx, Hb, m, fy, Vj, iu, bcO, bks, rt, cZ, dD, sw, aw, n, aj, fr, apt, uE, qN, zP, cd, asS, ai, yn, eW, b, ft, Xe, kl, h, nm, By, dL, ey, dd, vx, Ha, bwj, Nr, A, bwi, fJ, hQ, bwc, aq, po, kp, mo, dw, mb, cM, wU, op, bwd, Qy, ky, lX, dB, xO, Fi, bjd, ajd, JM, at, E, uz, XG, pg, bwg, it, B, arO, vQ, bL, fM, dA, atB, dk, fG, jx, aA, cz, v, j, C, sl, yV, AO, Eh, q, J, bag, er, eA, lO, qK, aT, cy, dz, Nc, Fx, zp, bq, lM, ae, bsq, af, dl, Lu, wr, s, iU, Ue, Y, oV, bg, Pg, ev, r, aYp, aSJ, en, hf, apx, t, Vq, iK, pb, wA, vk, amF, ck, sS, nM, iM, vZ, WP, hc, nG, DW, hr, adR, bwh, dy, fD, bt, rA, VB, vi, sY, id, qb, LX, ha, jh, vm, dG, oZ, eq, ad, aoH, bwf, aHF, dP, hd, av, db, Os, vw, aSf, hP, ji, vP, uX, d, lR, je, bwk, qt, ff, xs, wc, eH, cr, dN, hv, aFr, aF, si, VI, aiz, eZ, iR, bz, dr, Hp, Nq, LB, gk, es, mi, zr, bE, Hs, aS, bC, be, i, y, X, jr); #c1(FN1) c2(NP_997643) c3(fibronectin isoform 4 preproprotein) c4(265646) c5(265647, 265648, 265649, 265650, 265651, 265652, 265653, 265654) c6(265655, 265656) c7(bM, T, os, ea, if, al, fd, bB, aTy, zh, ci, lR, Lw, aao, x, Na, aE, ws, rJ, ael, us, azm, K, eL, bc, cp, aa, DA, eG, zQ, aeR, yo, aW, yK, hl, g, as, eE, eF, bwe, uC, cm, aAW, ba, lt, eM, nB, iA, iT, go, vA, mx, vy, cj, Gs, Zn, uJ, qr, wx, Hb, m, fy, Vj, iu, bcO, bks, rt, cZ, dD, sw, aw, n, aj, fr, apt, uE, qN, zP, cd, asS, ai, yn, eW, b, ft, Xe, kl, h, nm, By, dL, ey, dd, vx, Ha, bwj, Nr, A, bwi, fJ, hQ, bwc, aq, po, kp, mo, dw, mb, cM, wU, op, bwd, By, ky, lX, dB, xO, Fi, bjd, ajd, JM, at, E, uz, XG, pg, bwg, it, B, arD, vQ, bL, fM, dA, atB, dk, fG, jx, aA, cz, v, j, C, sl, yV, AO, Eh, q, J, bag, er, eA, lO, qK, aT, cy, dz, Nc, Fx, zp, bq, lM, ae, bsq, af, dl, Lu, wr, s, iU, Ue, Y, oV, bg, Pg, ev, r, aYp, aSJ, en, hf, apx, t, Vq, iK, pb, wA, vk, amF, ck, sS, nM, iM, vZ, WP, hc, nG, DW, hr, adR, bwh, dy, fD, bt, rA, VB, vi, sY, id, qb, LX, ha, jh, vm, dG, oZ, eq, ad, aoH, bwf, aHF, dP, hd, av, db, Os, vw, aSf, hP, ji, vP, uX, d, lR, je, bwk, qt, ff, xs, wc, eH, cr, dN, hv, aFr, aF, si, VI, aiz, eZ, iR, bz, dr, Hp, Nq, LB, gk, es, mi, zr, bE, Hs, aS, bC, be, i, y, X, jr); #c1(FN1) c2(NP_997647) c3 (fibronectin isoform 1 preproprotein) c4(265664) c5(265665, 265666, 265667, 265668, 265669, 265670, 265671, 265672) c6(265673, 265674) c7(bM, T, os, ea, if, al, fd, bB, aTy, zh, ci, lR, Lw, aao, x, Na, aE, ws, rJ, aeO, us, azm, K, eL, bc, cp, aa, DA, eG, zQ, aeR, yo, aW, yK, hl, g, as, eE, eF, bwe, uC, cm, aAW, ba, lt, eM, nB, iA, iT, go, vA, mx, vy, cj, Gs, Zn, uJ, qr, wx, Hb, m, fy, Vj, iu, bcO, bks, rt, cZ, dD, sw, aw, n, aj, fr, apt, uE, qN, zP, cd, asS, ai, yn, eW, b, ft, Xe, kl, h, nm, By, dL, ey, dd, vx, Ha, bwj, Nr, A, bwi, fJ, hQ, bwc, aq, po, kp, mo, dw, mb, cM, wU, op, bwd, By, ky, lX, dB, xO, Fi, bjd, ajd, JM, at, E, uz, XG, pg, bwg, it, B, arO, vQ, bL, fM, dA, atB, dk, fG, jx, aA, cz, v, j, C, sl, yV, AD, Eh, q, J, bag, er, eA, lO, qK, aT, cy, dz, Nc, Fx, zp, bq, lM, ae, bsq, af, dl, Lu, wr, s, iU, Ue, Y, oV, bg, Pg, ev, r, aYp, aSJ, en, hf, apx, t, Vq, iK, pb, wA, vk, amF, ck, sS, nM, iM, vZ, WP, hc, nG, DW, hr, adR, bwh, dy, fD, bt, rA, VB, vi, sY, id, qb, LX, ha, jh, vm, dG, oZ, eq, ad, aoH, bwf, aHF, dP, hd, av, db, Os, vw, aSf, hP, ji, vP, uX, d, lR, je, bwk, qt, ff, xs, wc, eH, cr, dN, hv, aFr, aF, si, VI, aiz, eZ, iR, bz, dr, Hp, Nq, LB, gk, es, mi, zr, bE, Hs, aS, bC, be, i, y, X, jr); #c1(FN1) c2(XP_005246455) c3(fibronectin isoform X2) c4(267156) c5( )c6( )c7(bM, T, os, ea, if, al, fd, bB, aTy, zh, ci, lR, Lw, aao, x, Na, aE, ws, rJ, aeO, us, azm, K, eL, bc, cp, aa, DA, eG, zQ, aeR, yo, aW, yK, hl, g, as, eE, eF, bwe, uC, cm, aAW, ba, lt, eM, nB, iA, iT, go, vA, mx, vy, cj, Gs, Zn, uJ, qr, wx, Hb, m, fy, Vj, iu, bcO, bks, rt, cZ, dD, sw, aw, n, aj, fr, apt, uE, qN, zP, cd, asS, ai, yn, eW, b, ft, Xe, kl, h, nm, By, dL, ey, dd, vx, Ha, bwj, Nr, A, bwi, fJ, hQ, bwc, aq, po, kp, mo, dw, mb, cM, wU, op, bwd, By, ky, lX, dB, xO, Fi, bjd, ajd, JM, at, E, uz, XG, pg, bwg, it, B, arD, vQ, bL, fM, dA, atB, dk, fG, jx, aA, cz, v, j, C, sl, yV, AO, Eh, q, J, bag, er, eA, lO, qK, aT, cy, dz, Nc, Fx, zp, bq, lM, ae, bsq, af, dl, Lu, wr, s, iU, Ue, Y, oV, bg, Pg, ev, r, aYp, aSJ, en, hf, apx, t, Vq, iK, pb, wA, vk, amF, ck, sS, nM, iM, vZ, WP, hc, nG, DW, hr, adR, bwh, dy, fD, bt, rA, VB, vi, sY, id, qb, LX, ha, jh, vm, dG, oZ, eq, ad, aoH, bwf, aHF, dP, hd, av, db, Os, vw, aSf, hP, ji, vP, uX, d, lR, je, bwk, qt, ff, xs, wc, eH, cr, dN, hv, aFr, aF, si, VI, aiz, eZ, iR, bz, dr, Hp, Nq, LB, gk, es, mi, zr, bE, Hs, aS, bC, be, i, y, X, jr); #c1(FN1) c2(XP_005246462) c3(fibronectin isoform X9) c4(267157) c5(267158, 267159, 267160, 267161, 267162, 267163, 267164, 267165) c6(267166, 267167) c7(bM, T, os, ea, if, al, fd, bB, aTy, zh, ci, lR, Lw, aao, x, Na, aE, ws, rJ, aeO, us, azm, K, eL, bc, cp, aa, DA, eG, zQ, aeR, yo, aW, yK, hl, g, as, eE, eF, bwe, uC, cm, aAW, ba, lt, eM, nB, iA, iT, go, vA, mx, vy, cj, Gs, Zn, uJ, qr, wx, Hb, m, fy, Vj, iu, bcO, bks, rt, cZ, dD, sw, aw, n, aj, fr, apt, uE, qN, zP, cd, asS, ai, yn, eW, b, ft, Xe, kl, h, nm, By, dL, ey, dd, vx, Ha, bwj, Nr, A, bwi, fJ, hQ, bwc, aq, po, kp, mo, dw, mb, cM, wl, op, bwd, y, ky, lX, dB, xO, Fi, bjd, ajd, JM, at, E, uz, XG, pg, bwg, it, B, arD, vQ, bL, fM, dA, atB, dk, fG, jx, aA, cz, v, j, C, sl, yV, AO, Eh, q, J, bag, er, eA, lO, qK, aT, cy, dz, Nc, Fx, zp, bq, lM, ae, bsq, af, dl, Lu, wr, s, il, Ue, Y, oV, bg, Pg, ev, r, aYp, aSJ, en, hf, apx, t, Vq, iK, pb, wA, vk, amF, ck, sS, nM, iM, vZ, WP, hc, nG, DW, hr, adR, bwh, dy, fD, bt, rA, VB, vi, sY, id, qb, LX, ha, jh, vm, dG, oZ, eq, ad, aoH, bwf, aHF, dP, hd, av, db, Os, vw, aSf, hP, ji, vP, uX, d, lR, je, bwk, qt, ff, xs, wc, eH, cr, dN, hv, aFr, aF, si, VI, aiz, eZ, iR, bz, dr, Hp, Nq, LB, gk, es, mi, zr, bE, Hs, aS, bC, be, i, y, X, jr); #c1(FN1) c2(XP_005246463) c3(fibronectin isoform X10) c4(267168, 267169) c5(267170, 267171, 267172, 267173, 267174, 267175, 267176, 267177, 267178, 267179, 267180, 267181, 267182, 267183, 267184, 267185) c6(267186, 267187, 267188, 267189) c7(bM, T, os, ea, if, al, fd, bB, aTy, zh, ci, lR, Lw, aao, x, Na, aE, ws, rJ, aeO, us, azm, K, eL, bc, cp, aa, DA, eG, zQ, aeR, yo, aW, yK, hl, g, as, eE, eF, bwe, uC, cm, aAW, ba, lt, eM, nB, iA, iT, go, vA, mx, vy, cj, Gs, Zn, uJ, qr, wx, Hb, m, fy, Vj, iu, bcO, bks, rt, cZ, dD, sw, aw, n, aj, fr, apt, uE, qN, zP, cd, asS, ai, yn, eW, b, ft, Xe, kl, h, nm, By, dL, ey, dd, vx, Ha, bwj, Nr, A, bwi, fJ, hQ, bwc, aq, po, kp, mo, dw, mb, cM, wl, op, bwd, By, ky, lX, dB, xO, Fi, bjd, ajd, JM, at, E, uz, XG, pg, bwg, it, B, arO, vQ, bL, fM, dA, atB, dk, fG, jx, aA, cz, v, j, C, sl, yV, AO, Eh, q, J, bag, er, eA, lO, qK, aT, cy, dz, Nc, Fx, zp, bq, lM, ae, bsq, af, dl, Lu, wr, s, iU, Ue, Y, oV, bg, Pg, ev, r, aYp, aSJ, en, hf, apx, t, Vq, iK, pb, wA, vk, amF, ck, sS, nM, iM, vZ, WP, hc, nG, DW, hr, adR, bwh, dy, fD, bt, rA, VB, vi, sY, id, qb, LX, ha, jh, vm, dG, oZ, eq, ad, aoH, bwf, aHF, dP, hd, av, db, Os, vw, aSf, hP, ji, vP, uX, d, lR, je, bwk, qt, ff, xs, wc, eH, cr, dN, hv, aFr, aF, si, VI, aiz, eZ, iR, bz, dr, Hp, Nq, LB, gk, es, mi, zr, bE, Hs, aS, bC, be, i, y, X, jr); #c1(FN1) c2(XP_005246464) c3(fibronectin isoform X11) c4(267190) c5( )c6( )c7(bM, T, os, ea, if, al, fd, bB, aTy, zh, ci, lR, Lw, aao, x, Na, aE, ws, rJ, ael, us, azm, K, eL, bc, cp, aa, DA, eG, zQ, aeR, yo, aW, yK, hl, g, as, eE, eF, bwe, uC, cm, aAW, ba, lt, eM, nB, iA, iT, go, vA, mx, vy, cj, Gs, Zn, uJ, qr, wx, Hb, m, fy, Vj, iu, bcO, bks, rt, cZ, dD, sw, aw, n, aj, fr, apt, uE, qN, zP, cd, asS, ai, yn, eW, b, ft, Xe, kl, h, nm, By, dL, ey, dd, vx, Ha, bwj, Nr, A, bwi, fJ, hQ, bwc, aq, po, kp, mo, dw, mb, cM, w, op, bwd, By, ky, lX, dB, xO, Fi, bjd, ajd, JM, at, E, uz, XG, pg, bwg, it, B, arD, vQ, bL, fM, dA, atB, dK, fG, jx, aA, cz, v, j, C, sl, yV, AO, Eh, q, J, bag, er, eA, lO, qK, aT, cy, dz, Nc, Fx, zp, bq, lM, ae, bsq, af, dl, Lu, wr, s, il, Ue, Y, oV, bg, Pg, ev, r, aYp, aSJ, en, hf, apx, t, Vq, iK, pb, wA, vk, amF, ck, sS, nM, lM, vZ, WP, hc, nG, DW, hr, adR, bwh, dy, fD, bt, rA, VB, vi, sY, id, qb, LX, ha, jh, vm, dG, oZ, eq, ad, aoH, bwf, aHF, dP, hd, av, db, Os, vw, aSf, hP, ji, vP, uX, d, lR, je, bwk, qt, ff, xs, wc, eH, cr, dN, hv, aFr, aF, si, VI, aiz, eZ, iR, bz, dr, Hp, Nq, LB, gk, es, mi, zr, bE, Hs, aS, bC, be, i, y, X, jr); #c1(FN1) c2(XP_005246471) c3(fibronectin isoform X18) c4(267191, 267192, 267193) c5(267194, 267195, 267196, 267197, 267198, 267199, 267200, 267201, 267202, 267203, 267204, 267205, 267206, 267207, 267208, 267209, 267210, 267211, 267212, 267213, 267214, 267215, 267216, 267217) c6(267218, 267219, 267220, 267221, 267222, 267223) c7(bM, T, os, ea, if, al, fd, bB, aTy, zh, ci, lR, Lw, aao, x, Na, aE, ws, rJ, aeO, us, azm, K, eL, bc, cp, aa, DA, eG, zQ, aeR, yo, aW, yK, hl, g, as, eE, eF, bwe, uC, cm, aAW, ba, lt, eM, nB, iA, iT, go, vA, mx, vy, cj, Gs, Zn, uJ, qr, wx, Hb, m, fy, Vj, iu, bcO, bks, rt, cZ, dD, sw, aw, n, aj, fr, apt, uE, qN, zP, cd, asS, ai, yn, eW, b, ft, Xe, kl, h, nm, By, dL, ey, dd, vx, Ha, bwj, Nr, A, bwi, fJ, hQ, bwc, aq, po, kp, mo, dw, mb, cM, wU, op, bwd, Qy, ky, lX, dB, xO, Fi, bjd, ajd, JM, at, E, uz, XG, pg, bwg, it, B, arD, vQ, bL, fM, dA, atB, dk, fG, jx, aA, cz, v, j, C, sl, yV, AO, Eh, q, J, bag, er, eA, lO, qK, aT, cy, dz, Nc, Fx, zp, bq, lM, ae, bsq, af, dl, Lu, wr, s, il, Ue, Y, oV, bg, Pg, ev, r, aYp, aSJ, en, hf, apx, t, Vq, iK, pb, wA, vk, amF, ck, sS, nM, iM, vZ, WP, hc, nG, DW, hr, adR, bwh, dy, fD, bt, rA, VB, vi, sY, id, qb, LX, ha, jh, vm, dG, oZ, eq, ad, aoH, bwf, aHF, dP, hd, av, db, Os, vw, aSf, hP, ji, vP, uX, d, lR, je, bwk, qt, ff, xs, wc, eH, cr, dN, hv, aFr, aF, si, VI, aiz, eZ, iR, bz, dr, Hp, Nq, LB, gk, es, mi, zr, bE, Hs, aS, bC, be, i, y, X, jr); #c1(FN3K) c2(NP_071441) c3(fructosamine-3-kinase) c4(211673, 211674, 211675) c5(211676, 211677, 211678, 211679, 211680, 211681, 211682, 211683, 211684, 211685, 211686, 211687, 211688, 211689, 211690, 211691, 211692, 211693) c6( )c7(bL, T, r, aT, aj, dG, q, J); #c1(FN3KRP) c2(NP_078895) c3(ketosamine-3-kinase) c4(215965, 215966) c5(215967, 215968, 215969, 215970, 215971, 215972, 215973, 215974, 215975, 215976, 215977, 215978) c6( )c7(e); #c1(FNBP1) c2(NP_055848) c3(formin-binding protein 1) c4(183906, 183907, 183908) c5(183909, 183910, 183911, 183912, 183913, 183914, 183915, 183916, 183917, 183918, 183919, 183920, 183921, 183922, 183923, 183924, 183925, 183926) c6( )c7(ck, bt); #c1(FNBP1) c2(XP_005251882) c3 (formin-binding protein 1 isoform X13) c4(271255) c5(271256, 271257, 271258, 271259, 271260, 271261) c6( )c7(ck, bt); #c1(FNBP1) c2(XP_005251883) c3(formin-binding protein 1 isoform X14) c4(271262) c5(271263, 271264, 271265, 271266, 271267, 271268) c6( )c7(ck, bt); #c1(FNBP1) c2(XP_005251886) c3(formin-binding protein 1 isoform X17) c4(27/269) c5(271270, 271271, 271272, 271273, 271274, 271275) c6( )c7(ck, bt); #c1(FNBP1) c2(XP_011516704) c3(formin-binding protein 1 isoform X4) c4(310151) c5(310152, 310153, 310154, 310155, 310156, 310157) c6( )c7(ck, bt); #c1(FNDC1) c2(NP_115921) c3(fibronectin type III domain-containing protein 1 precursor) c4(224703) c5(224704, 224705, 224706, 224707, 224708, 224709, 224710) c6( )c7(j, Ea); #c1(FNDC1) c2(XP_011534493) c3 (fibronectin type III domain-containing protein 1 isoform X2) c4(330950) c5(330951, 330952, 330953, 330954, 330955, 330956) c6( )c7(j, Ea); #c1(FNDC3A) c2(NP_001073141) c3(fibronectin type-Ill domain-containing protein 3A isoform 1) c4(35608, 35609, 35610) c5(35611, 35612, 35613, 35614, 35615, 35616, 35617, 35618, 35619, 35620, 35621, 35622, 35623, 35624, 35625, 35626, 35627, 35628, 35629, 35630, 35631, 35632) c6( )c7(ae, y, c, WY, vA, md, aaE, bk, aS, MC, rz, bt, bz, bgv); #c1(FNDC3A) c2(NP_055738) c3(fibronectin type-Ill domain-containing protein 3A isoform 2) c4(183196) c5(183197, 183198, 183199, 183200, 183201, 183202) c6( )c7(ae, y, c, WY, vA, mQ, aaE, bK, aS, MC, rz, bt, bz, bgv); #c1(FNDC3B) c2(NP_001128567) c3(fibronectin type III domain-containing protein 3B) c4(50251, 50252) c5(50253, 50254, 50255, 50256, 50257, 50258, 50259, 50260, 50261, 50262, 50263, 50264, 50265, 50266, 50267, 50268, 50269, 50270, 50271, 50272) c6( )c7(ab, T, B, j, dH); #c1(FNDC3B) c2(XP_011511385) c3(fibronectin type III domain-containing protein 3B isoform X1) c4(301957) c5(301958, 301959, 301960, 301961, 301962, 301963, 301964) c6( )c7(ab, T, B, j, dH); #c1(FNDC4) c2(NP_073734) c3(fibronectin type III domain-containing protein 4 precursor) c4(213312) c5(213313, 213314, 213315, 213316, 213317, 213318) c6( )c7(gv, du); #c1(FNTA) c2(NP_002018) c3 (protein farnesyltransferase/geranylgeranyltransferase type-1 subunit alpha) c4(123345, 123346) c5(123347, 123348, 123349, 123350, 123351, 123352, 123353, 123354, 123355, 123356, 123357, 123358) c6( )c7(om, T); #c1(FNTB) c2(NP_002019) c3(protein farnesyltransferase subunit beta) c4(123359) c5(123360, 123361, 123362, 123363, 123364, 123365) c6( )c7(om); #c1(FOCAD) c2(NP_060264) c3(focadhesin) c4(195127) c5(195128, 195129, 195130, 195131, 195132, 195133, 195134, 195135, 195136, 195137) c6( )c7(cd, e, d, eA, af); #c1(FOLH1) c2(NP_001014986) c3(glutamate carboxypeptidase 2 isoform 2) c4(26099, 26100) c5(26101, 26102, 26103, 26104, 26105, 26106) c6( )c7(ng, T, eR, e, aw, fd, aA, sM, n, cz, bS, j, ct, gf, ci, Fb, acU, q, ad, aZJ, x, Na, gH, aT, Nc, K, ae, eu, bwl, aW, r, lo, CO, g, Z, kk, bz, hq, es, ac, iA, bP, lp, wE, bm, at, k, Tw, gg, LS); #c1(FOLH1) c2(NP_001180400) c3(glutamate carboxypeptidase 2 isoform 3) c4(73939) c5(73940, 73941, 73942, 73943, 73944, 73945) c6( )c7(ng, T, eR, e, aw, fd, aA, sM, n, cz, bS, j, ct, gf, ci, Fb, acU, q, ad, aZJ, x, Na, gH, aT, Nc, K, ae, eu, bwl, aW, r, lo, CO, g, Z, kk, bz, hq, es, ac, iA, bP, lp, wE, bm, at, k, Tw, gg, LS); #c1(FOLH1) c2(NP_001180401) c3(glutamate carboxypeptidase 2 isoform 4) c4(73946) c5(73947, 73948, 73949, 73950, 73951, 73952) c6( )c7(ng, T, eR, e, aw, fd, aA, sM, n, cz, bS, j, ct, gf, ci, Fb, acU, q, ad, aZJ, x, Na, gH, aT, Nc, K, ae, eu, bwl, aW, r, lo, CO, g, Z, kk, bz, hq, es, ac, iA, bP, lp, wE, bm, at, k, Tw, gg, LS); #c1(FOLH1) c2(NP_00180402) c3(glutamate carboxypeptidase 2 isoform 5) c4(73953) c5( )c6( )c7(ng, T, eR, e, aw, fd, aA, sM, n, cz, bS, j, ct, gf, ci, Fb, acU, q, ad, aZJ, x, Na, gH, aT, Nc, K, ae, eu, bwl, aW, r, lo, CO, g, Z, kk, bz, hq, es, ac, iA, bP, lp, wE, bm, at, k, Tw, gg, LS); #c1(FOLH1) c2(NP_004467) c3(glutamate carboxypeptidase 2 isoform 1) c4(146593) c5(146594, 146595, 146596, 146597, 146598, 146599, 146600, 146601, 146602, 146603, 146604, 146605, 146606, 146607, 146608, 146609, 146610, 146611) c6( )c7(ng, T, eR, e, aw, fd, aA, sM, n, cz, bS, j, ct, gf, ci, Fb, acU, q, ad, aZJ, x, Na, gH, aT, Nc, K, ae, eu, bwl, aW, r, lo, CQ, g, Z, kk, bz, hq, es, ac, iA, bP, lp, wE, bm, at, k, Tw, gg, LS); #c1(FOLR1) c2(NP_000793) c3 (folate receptor alpha precursor) c4(16116) c5(16117, 16118, 16119, 16120, 16121, 16122, 16123, 16124, 16125, 16126, 16127, 16128, 16129, 16130, 16131, 16132, 16133, 16134) c6( )c7(ng, T, ha, aA, ci, nA, Na, iu, ib, hw, hb, btl, bwo, aT, Nc, cx, x, yr, MX, bwn, bwm, aW, Y, vj, eH, Az, ff, r, aq, en, g, eF, t, xw, bz, btb, ck, Qi, eM, Ip, wE, X, bwp, U, hR, m, bD, y, bt, B, hS); #c1(FOLR2) c2(NP_001107008) c3(folate receptor beta precursor) c4( )c5(42788, 42789, 42790, 42791, 42792, 42793, 42794, 42795, 42796, 42797, 42798) c6( )c7(LB, Qi, Na, lp, fG, vj, eb, Nc, aS, bD, y, bt, lU); #c1(FOLR2) c2(XP_011543171) c3(folate receptor beta isoform X1) c4(342001, 342002) c5(342003, 342004, 342005, 342006, 342007, 342008, 342009) c6( )c7(LB, Qi, Na, lp, fG, vj, eb, Nc, aS, bD, y, bt, lU); #c1(FOLR2) c2(XP_011543172) c3(folate receptor beta isoform X2) c4(342010) c5(342011) c6( )c7(LB, Di, Na, lp, fG, vj, eb, Nc, aS, bD, y, bt, lU); #c1(FOLR3) c2(NP_000795) c3(folate receptor gamma precursor) c4(16135, 16136) c5(16137, 16138, 16139, 16140, 16141, 16142, 16143) c6( )c7(Na, ib, lp, aW, lU, aT, Nc, m, hS); #c1(FOPNL) c2(NP_653201) c3(lisH domain-containing protein FOPNL isoform 1) c4(239043) c5(239044, 239045, 239046, 239047, 239048, 239049) c6( )c7(bL, T, kh, dD, qb, ea, aw, jx, aA, fd, n, aj, v, eq, ci, acU, iJ, q, ad, hX, iC, mj, F, Uh, aT, h, cy, ae, jf, A, BO, aW, xi, oV, ih, fc, eZ, r, g, cl, cm, dr, bN, ck, bm, wq, adg, fh, B); #c1(FOS) c2(NP_005243) c3(proto-oncogene c-Fos) c4(154348) c5(154349, 154350, 154351, 154352, 154353, 154354, 154355) c6( )c7(bM, T, fR, ea, if, e, bO, bB, ci, amW, aR, ip, x, xE, aE, dV, Ds, K, eL, xa, aHE, aa, eG, aW, Ls, az, g, eE, eF, cm, bw, eM, nB, lp, iT, bwr, vA, wB, cj, k, m, iu, dD, lT, bws, nA, PJ, qN, cd, br, ai, adf, rC, Nn, h, lG, dL, uV, A, anP, aq, op, hq, kT, GU, hk, hL, at, cB, B, vQ, Dw, NO, dA, fG, yk, rf, aA, cz, hz, v, j, C, q, eA, nv, aT, jj, ae, af, kl, bf, s, il, Y, r, cu, lo, tu, en, t, iK, pb, ck, nM, iM, hc, CZ, fD, zu, bt, ao, wi, Ke, qb, aZ, aD, ha, jh, dG, eq, ad, bwq, dP, av, hd, F, Yi, ul, gj, d, je, ff, eH, cr, eZ, gl, tv, aF, si, EZ, iV, adj, bz, ek, iq, bF, bE, aS, aGg, lC, KS, CW, y); #c1 (FOSB) c2(NP_001107643) c3(protein fosB isoform 2) c4(42926) c5(42927, 42928, 42929, 42930, 42931, 42932, 42933, 42934, 42935, 42936, 42937, 42938) c6( )c7(bM, T, aUk, if, e, bB, ci, x, aE, K, eL, aa, eG, aW, az, CT, eE, qJ, eM, nB, lT, wB, k, m, ct, qN, ai, h, dL, anP, aq, cv, kT, ok, GU, B, vQ, NO, yk, rf, aA, cz, hz, v, C, q, eA, nv, aT, ae, af, bf, Y, r, cu, lo, tu, en, t, xw, iK, pb, bN, QG, iM, hc, CZ, fD, bt, ao, Ke, qb, aD, ha, bS, dG, ad, hd, Yi, gj, d, je, ff, cr, eZ, tv, af, si, Z, bz, ek, bE, aS, lC, iu, y); #c1(FOSB) c2(NP_006723) c3(protein fosB isoform 1) c4(167437, 167438) c5(167439, 167440, 167441, 167442, 167443, 167444, 167445) c6( )c7(bM, T, aUk, if, e, bB, ci, x, aE, K, eL, aa, eG, aW, az, CT, eE, qJ, eM, nB, iT, wB, k, m, ct, qN, ai, h, dl, anP, aq, cv, kT, ok, GU, B, vQ, NO, yk, rf, aA, cz, hz, v, C, q, eA, nv, aT, ae, af, bf, Y, r, cu, lo, tu, en, t, xw, iK, pb, bN, QG, iM, hc, CZ, fD, bt, ao, Ke, qb, aD, ha, bS, dG, ad, hd, Yi, gj, d, je, ff, cr, eZ, tv, aF, si, Z, bz, ek, bE, aS, lC, iu, y); #c1(FOSB) c2(XP_005258748) c3(protein fosB isoform X1) c4(275720) c5(275721, 275722, 275723, 275724, 275725, 275726) c6( )c7(bM, T, aUk, if, e, bB, ci, x, aE, K, eL, aa, eG, aW, az, CT, eE, qJ, eM, nB, iT, wB, k, m, ct, qN, ai, h, dl, anP, aq, cv, kT, ok, GU, B, vQ, NO, yk, rf, aA, cz, hz, v, C, q, eA, nv, aT, ae, af, bf, Y, r, cu, lo, tu, en, t, xw, iK, pb, bN, QG, iM, hc, CZ, fD, bt, ao, Ke, qb, aD, ha, bS, dG, ad, hd, Yi, gj, d, je, ff, cr, eZ, tv, aF, si, Z, bz, ek, bE, aS, lC, iu, y); #c1(FOSL1) c2(NP_001287784) c3(fos-related antigen 1 isoform 3) c4( )c5(112864) c6( )c7(bM, T, qo, fG, if, bB, hz, fr, ci, nA, qN, cd, q, ad, eA, Vp, av, hd, Vr, aT, lG, dL, Fx, ey, ae, bc, eL, pl, d, s, aW, Y, ff, r, Ku, en, g, zE, t, bz, as, bF, es, kP, eM, nB, bE, iA, m, it, X); #c1(FOSL1) c2(NP_001287786) c3(fos-related antigen 1 isoform 5) c4(112865) c5(112866, 112867, 112868, 112869, 112870, 112871) c6( )c7(bM, T, qo, fG, if, bB, hz, fr, ci, nA, qN, cd, q, ad, eA, Vp, av, hd, Vr, aT, lG, dL, Fx, ey, ae, bc, eL, pl, d, s, aW, Y, ff, r, Ku, en, g, zE, t, bz, as, bF, es, kP, eM, nB, bE, iA, m, it, X); #c1(FOSL1) c2(NP_005429) c3 (fos-related antigen 1 isoform 1) c4(156095, 156096) c5(156097, 156098, 156099, 156100, 156101, 156102, 156103, 156104, 156105, 156106, 156107, 156108, 156109) c6( )c7(bM, T, qo, fG, if, bB, hz, fr, ci, nA, qN, cd, q, ad, eA, Vp, av, hd, Vr, aT, lG, dL, Fx, ey, ae, bc, eL, pl, d, s, aW, Y, ff, r, Ku, en, g, zE, t, bz, as, bF, es, kP, eM, nB, bE, iA, m, it, X); #c1(FOSL2) c2(NP_005244) c3(fos-related antigen 2) c4(154356, 154357) c5(154358, 154359, 154360, 154361, 154362, 154363, 154364, 154365, 154366, 154367, 154368, 154369, 154370) c6( )c7(ID, T, fG, ci, ad, BY, aE, aT, lG, an, ae, A, je, aW, Y, ev, cl, t, pb, LB, es, iM, aS, m, bt); #c1(FOXA1) c2(NP_004487) c3(hepatocyte nuclear factor 3-alpha) c4(146839, 146840) c5(146841, 146842, 146843, 146844, 146845, 146846, 146847, 146848, 146849, 146850, 146851, 146852, 146853) c6( ) c7(T, ht, n, ci, AO, ad, av, Yn, hd, aE, aT, K, hu, a, bA, aW, ff, fc, aef, aq, kp, en, cl, g, bz, nB, hL, B); #c1(FOXA2) c2(NP_068556) c3(hepatocyte nuclear factor 3-beta isoform 1) c4(209407, 209408) c5(209409, 209410, 209411, 209412, 209413, 209414, 209415, 209416, 2094/7) c6( )c7(bL, T, jx, fF, aj, J, ad, av, aE, bq, ae, aa, AQ, hl, aeF, aq, kp, g, bz, LB, nM, eM, Jr, nB, wk, nG, bo, ZW, B, Ex); #c1(FOXA2) c2(XP_006723625) c3(hepatocyte nuclear factor 3-beta isoform X1) c4( )c5(295260, 295261, 295262, 295263, 295264, 295265, 295266, 295267) c6( )c7(bL, T, jx, fF, aj, J, ad, av, aE, bq, ae, aa, AQ, hl, aeF, aq, kp, g, bz, LB, nM, eM, Jr, nB, wk, nG, bo, ZW, B, Ex); #c1(FOXA2) c2(XP_011527533) c3(hepatocyte nuclear factor 3-beta isoform X2) c4(322347) c5(322348, 322349, 322350, 322351, 322352, 322353) c6( )c7(bL, T, jx, fF, aj, J, ad, av, aE, bq, ae, aa, AQ, hl, aeF, aq, kp, g, bz, LB, nM, eM, Jr, nB, wk, nG, bo, ZW, B, Ex); #c1(FOXA3) c2(NP_004488) c3(hepatocyte nuclear factor 3-gamma) c4(146854) c5(146855, 146856, 146857, 146858, 146859, 146860) c6( )c7(aq, B, aa, ad, bq); #c1(FOXB1) c2(NP_036314) c3(forkhead box protein B1) c4(172966) c5(172967, 172968, 172969, 172970, (72971, 172972) c6( )c7(dY, sj); #c1(FOXC1) c2(NP_001444) c3(forkhead box protein CI) c4(117869, 117870) c5(117871, 117872, 117873, 117874, 117875, 117876, 117877, 117878, 117879, 117880, 117881, 117882) c6( )c7(bL, T, ul, JJ, pQ, bbH, bwt, n, aj, ko, jE, nA, aSL, Xr, cd, J, bwu, ad, Mg, anA, asY, ath, bki, aT, Vv, dL, K, anz, rz, bbG, ae, gc, bw, aW, eZ, kp, cl, dw, t, Ye, ck, eM, KB, hL, at, bbF, Hb, m, y, bt, B); #c1(FOXC2) c2(NP_005242) c3(forkhead box protein C2) c4(154341) c5(154342, 154343, 154344, 154345, 154346, 154347) c6( )c7(bL, T, if, e, ht, bbH, n, v, qN, bwx, q, Hr, Mg, Na, bbp, acA, bwy, cE, aT, Vv, hu, aiJ, kl, aW, ff, Az, r, yy, zC, bww, en, dw, bz, dr, bJ, alu, eM, mg, PO, PN, ei, it); #c1(FOXD1) c2(NP_004463) c3(forkhead box protein DI) c4(146562) c5(146563, 146564, 146565, 146566, 146567, 146568, 146569) c6( )c7(aDM); #c1(FOXE1) c2(NP_004464) c3(forkhead box protein E1) c4(146570) c5(146571, 146572, 146573, 146574, 146575, 146576, 146577, 146578) c6( )c7(T, aMW, v, q, aR, Na, hd, aE, db, il, RN, nX, Nc, ji, aDX, aoa, bwB, ff, bfu, r, en, bwA, zZ, sS, apM, nM, nB, adg, gT, bwz); #c1(FOXF1) c2(NP_001442) c3(forkhead box protein Fl) c4(117854) c5(117855, 117856, 117857, 117858, 117859, 117860, (17861) c6( )c7(T, pf, sw, fG, nA, AD, q, bv, acA, aT, Vv, hu, rz, aa, qt, aW, ap, r, fH, aDP, g, bz, LB, ac, ajd, Tc); #c1(FOXF2) c2(NP_001443) c3(forkhead box protein F2) c4(117862) c5(117863, 117864, 117865, 117866, 117867, 117868) c6( )c7(T, kP, c, aW, aT, cz, cm, ae, ad); #c1 (FOXG1) c2(NP_005240) c3(forkhead box protein GI) c4(154320) c5(154321, 154322, 154323, 154324, 154325, 154326, 154327, 154328, 154329, 154330, 154331) c6( )c7(jf, Qi, iO, alW, QK, bwC, hb, aW, ha, cC, aT, m, nA, WI, t, bwD, ZW, aR, bN); #c1(FOXH1) c2(NP_003914) c3(forkhead box protein H1) c4(142123, 142124) c5(142125, 142126, 142127, 142128, 142129, 142130, 142131, 142132, 142133, 142134, 142135, 142136, 142137, 142138) c6( )c7(acl, bwE, ZW, bD, ae, ad, wT); #c1(FOXI1) c2(NP_036320) c3(forkhead box protein I1 isoform a) c4(172973) c5(172974, 172975, 172976, 172977, 172978, 172979, 172980) c6( )c7(bwf, ale, Az, bwG); #c1(FOXI1) c2(NP_658982) c3(forkhead box protein 11 isoform b) c4(239613) c5(239614, 239615, 239616, 239617, 239618, 239619) c6( )c7(bwF, ale, Az, bwG); #c1(FOXJ1) c2(NP_001445) c3(forkhead box protein JI) c4(117883) c5(117884, 117885, 117886, 117887, 117888, 117889, (17890) c6( )c7(aE, afm, aW, CM, JM, aT, cU, kt, lo, eZ, K, m, fl, t, mC, y); #c1(FOXJ2) c2(NP_060886) c3(forkhead box protein J2) c4(198378) c5(198379, 198380, 198381, 198382, 198383, 198384, 198385, 198386, 198387, 198388, 198389, 198390, 198391, 198392, 198393) c6(198394, 198395) c7(Vv, aW, aT); #c1(FOXJ2) c2(XP_011519063) c3(forkhead box protein J2 isoform X1) c4(313664) c5( ) c6( )c7(Vv, aW, aT); #c1(FOXK1) c2(NP_001032242) c3(forkhead box protein KI) c4(31152, 31153, 31154) c5(31155, 31156, 31157, 31158, 31159, 31160, 31161, 31162, 31163, 31164, 31165, 31166, 31167) c6( )c7(bwH); #c1(FOXK1) c2(XP_011513493) c3(forkhead box protein KI isoform X1) c4(305777) c5(305778, 305779, 305780, 305781, 305782, 305783, 305784, 305785, 305786, 305787, 305788) c6( ) c7(bwH); #c1(FOXK2) c2(NP_004505) c3(forkhead box protein K2) c4(146976, 146977, 146978) c5(146979, 146980, 146981, 146982, 146983, 146984, 146985, 146986, 146987, 146988, 146989, 146990, 146991) c6( )c7(bwH); #c1(FOXK2) c2(XP_011521875) c3(forkhead box protein K2 isoform X1) c4( )c5(316474, 316475, 316476, 316477, 316478, 316479) c6( )c7(bwH); #c1(FOXL1) c2(NP_005241) c3(forkhead box protein L1) c4(154332) c5(154333, 154334, 154335, 154336, 154337, 154338, 154339, 154340) c6( )c7(aC, aE, BJ, Vv, kp, t, af); #c1(FOXL2) c2(NP_075555) c3(forkhead box protein L2) c4(213934, 213935) c5(213936, 213937, 213938, 213939, 213940, 213941, 213942, 213943, 213944, 213945, 213946, 213947) c6( )c7(T, aAz, n, aTS, nA, aOr, zW, bwl, anB, vJ, il, nX, arH, BJ, aOq, PI, K, uc, ako, bwM, il, bwK, bwl, yo, s, asP, alB, xv, baZ, bwl, g, bwJ, t, zZ, QK, bfw, dr, arB, avN, aNy, aXt, bwN); #c1(FOXM1) c2(NP_068772) c3(forkhead box protein MI isoform 2) c4(210322) c5(210323, 210324, 210325, 210326, 210327, 210328, 210329) c6( )c7(T, aC, fM, dA, jp, pf, fG, yk, if, qy, aA, bB, n, v, ci, qN, q, ad, ai, hX, eA, bv, av, hd, F, aE, lg, mj, iu, py, jz, anV, aT, dL, hP, K, ey, KV, ae, eL, dx, pl, jf, A, d, s, aW, ff, az, eH, r, aq, Ej, kp, aF, en, g, eF, t, cm, bz, ik, asE, oU, bwl, ck, bF, sS, nM, kP, eM, nB, dB, bE, np, m, bwP, fD, bt, B, cn, hS); #c1(FOXM1) c2(NP_973731) c3(forkhead box protein M1 isoform 1) c4(262701) c5(262702, 262703, 262704, 262705, 262706, 262707) c6( )c7(T, aC, fM, dA, jp, pf, fG, yk, if, qy, aA, bB, n, v, ci, qN, q, ad, ai, hX, eA, bv, av, hd, F, aE, lg, mj, iu, py, jz, anV, aT, dl, hP, K, ey, KV, ae, eL, dx, pl, jf, A, d, s, aW, ff, az, eH, r, aq, Ej, kp, aF, en, g, eF, t, cm, bz, ik, asE, ou, bwl, ck, bF, sS, nM, kP, eM, nB, dB, bE, np, m, bwP, fD, bt, B, cn, hS); #c1(FOXM1) c2(NP_973732) c3 (forkhead box protein M1 isoform 3) c4(262708) c5(262709, 262710, 262711, 262712, 262713, 262714) c6( )c7(T, aC, fM, dA, jp, pf, fG, yk, if, qy, aA, bB, n, v, ci, qN, q, ad, ai, hX, eA, bv, av, hd, F, aE, lg, mj, iu, py, jz, anV, aT, dL, hP, K, ey, KV, ae, eL, dx, pl, jf, A, d, s, aW, ff, az, eH, r, aq, Ej, kp, aF, en, g, eF, t, cm, bz, ik, asE, oU, bwl, ck, bF, sS, nM, kP, eM, nB, dB, bE, np, m, bwP, fD, bt, B, cn, hS); #c1(FOXN1) c2(XP_005258103) c3(forkhead box protein NI isoform X1) c4( )c5(275494, 275495, 275496, 275497, 275498, 275499, 275500, 275501) c6( )c7(T, dA, lp, Y, RX, yk, ev, ff, aeM, bwS, YD, ci, bwR, en, pd, ae, Zm, bwT); #c1(FOXN1) c2(XP_011523658) c3(forkhead box protein NI isoform X4) c4(318162) c5(318163) c6( )c7(T, dA, lp, Y, RX, yk, ev, ff, aeM, bwS, YD, ci, bwR, en, pd, ae, Zm, bwT); #c1(FOXN1) c2(XP_011523672) c3(forkhead box protein NI isoform X13) c4( )c5(318164) c6( )c7(T, dA, lp, Y, RX, yk, ev, ff, aeM, bwS, YD, ci, bwR, en, pd, ae, Zm, bwT); #c1(FOXN2) c2(NP_002149) c3(forkhead box protein N2) c4(125941, 125942, 125943) c5(125944, 125945, 125946, 125947, 125948, 125949, 125950, 125951, 125952, 125953, 125954, 125955, 125956, 125957, 125958, 125959, 125960, 125961) c6( )c7(je, iM); #c1(FOXN3) c2(NP_001078940) c3(forkhead box protein N3 isoform 1) c4(38137) c5(38138, 38139, 38140, 38141, 38142, 38143, 38144, 38145, 38146, 38147) c6( )c7(B, iT, iZ, aw, fq, cd); #c1(FOXN3) c2(NP_005188) c3(forkhead box protein N3 isoform 2) c4(153747, 153748, 153749, 153750) c5(153751, 153752, 153753, 153754, 153755, 153756, 153757, 153758, 153759, 153760, 153761, 153762, 153763, 153764, 153765, 153766, 153767, 153768, 153769, 153770, 153771, 153772, 153773, 153774, 153775, 153776, 153777, 153778, 153779) c6( )c7(B, iT, iZ, aw, fq, cd); #c1(FOX01) c2(NP_002006) c3(forkhead box protein 01) c4(123193) c5(123194, 123195, 123196, 123197, 123198, 123199, 123200, 123201) c6( )c7(T, aC, bwl, if, bB, bv, aE, il, K, aW, fc, cm, eM, nB, AM, iT, vA, m, Xu, dD, aj, qN, jz, jy, eV, bl, dx, aq, zZ, op, bJ, bP, eD, hL, at, fL, B, bL, fM, dA, jx, eX, fZ, cR, aA, hy, v, j, eA, J, aT, kf, ae, ne, kl, Or, iU, oV, ev, cl, t, OC, rR, bnR, mf, aps, bt, aZ, iW, dG, eq, mC, ad, dP, py, ab, iY, bwV, d, BN, eZ, aF, bz, bF, bE, cP, ei, lC, iu, y); #c1(FOX03) c2(NP_963853) c3(forkhead box protein 03) c4(262433) c5(262434, 262435, 262436, 262437, 262438, 262439, 262440, 262441, 262442, 262443) c6( )c7(oO, gh, T, aAx, EQ, bB, ci, x, xE, il, dV, K, dF, bc, eL, aW, fc, Qq, hN, cm, qJ, eM, nB, iA, vA, cj, m, k, dD, aw, h, hn, dl, bl, aq, jT, Z, op, bJ, bP, cA, Qu, at, B, Dh, jx, aA, v, j, AD, V, pd, q, eA, cF, qK, aT, cy, rO, bK, jj, bq, ae, iU, Y, aeS, r, lo, hf, t, ik, ck, rR, fD, bt, bwW, jh, eq, EA, ad, hd, py, iY, ji, d, qt, eZ, aF, bz, LB, es, bE, apv, y, iu); #c1(FOX03) c2(XP_005266924) c3(forkhead box protein 03 isoform X3) c4(280404) c5(280405) c6( )c7(oO, gh, T, aAx, EQ, bB, ci, x, xE, il, dV, K, dF, bc, eL, aW, fc, Qq, hN, cm, qJ, eM, nB, iA, vA, cj, m, k, dD, aw, h, hn, dL, bl, aq, jT, zZ, op, bJ, bP, cA, Qu, at, B, Dh, jx, aA, v, j, AD, V, pd, q, eA, cF, qK, aT, cy, rO, bK, jj, bq, ae, iU, Y, aeS, r, lo, hf, t, iK, ck, rR, fD, bt, bwW, jh, eq, EA, ad, hd, py, iY, ji, d, qt, eZ, aF, bz, LB, es, bE, apv, y, iu); #c1(FOX03) c2(XP_011533928) c3(forkhead box protein 03 isoform X1) c4( ) c5(330118) c6( )c7(oO, gh, T, aAx, EQ, bB, ci, x, xE, il, dV, K, dF, bc, eL, aW, fc, Qq, hN, cm, qJ, eM, nB, iA, vA, cj, m, k, dD, aw, h, hn, dL, bl, aq, jT, zZ, op, bJ, bP, cA, Qu, at, B, Dh, jx, aA, v, j, AD, V, pd, q, eA, cF, qK, aT, cy, rO, bK, jj, bq, ae, il, Y, aeS, r, lo, hf, t, iK, ck, rR, fD, bt, bwW, jh, eq, EA, ad, hd, py, iY, ji, d, qt, eZ, aF, bz, LB, es, bE, apv, y, iu); #c1(FOX03) c2(XP_011533931) c3(forkhead box protein 03 isoform X4) c4(330119) c5(330120, 330121, 330122, 330123, 330124, 330125, 330126, 330127, 330128, 330129) c6( )c7(oO, gh, T, aAx, EQ, bB, ci, x, xE, il, dV, K, dF, bc, eL, aW, fc, Qq, hN, cm, qJ, eM, nB, iA, vA, cj, m, k, dD, aw, h, hn, dL, bl, aq, jT, zZ, op, bJ, bP, cA, Qu, at, B, Dh, jx, aA, v, j, AD, V, pd, q, eA, cF, qK, aT, cy, rQ, bK, jj, bq, ae, il, Y, aeS, r, lo, hf, t, ik, ck, rR, fD, bt, bwW, jh, eq, EA, ad, hd, py, iY, ji, d, qt, eZ, aF, bz, LB, es, bE, apv, y, iu); #c1(FOX04) c2(NP_001164402) c3 (forkhead box protein 04 isoform 2) c4(66921) c5(66922, 66923, 66924, 66925, 66926, 66927) c6( )c7(T, y, bP, Kv, il, SV, aA, eZ, aj, v, aF, K, k, aiG, hF, bt, J, bJ); #c1(FOX04) c2(NP_005929) c3(forkhead box protein 04 isoform 1) c4(160495) c5(160496, 160497, 160498, 160499, 160500, 160501, 160502) c6( )c7(T, y, bP, Kv, il, SV, aA, eZ, aj, v, aF, K, k, aiG, hF, bt, J, bJ); #c1(FOX06) c2(NP_001278210) c3(forkhead box protein 06) c4(109490) c5(109491, 109492, 109493, 109494, 109495, 109496, 109497) c6( )c7(bL, aA, aj, dG, bK, v, aF, avf, J); #c1(FOXP1) c2(NP_001012523) c3 (forkhead box protein P1 isoform 2) c4(25019) c5(25020, 25021, 25022, 25023, 25024, 25025, 25026) c6( )c7(T, aC, fM, pf, sw, VT, aA, bD, bwX, jh, j, ci, nA, qj, ad, anU, x, yt, acA, zK, aT, LA, cy, Ko, KU, he, hH, ake, ae, rz, d, VX, BO, aW, eH, bwY, eF, t, bN, aUn, rR, yl, QG, eM, au, X, cj, MW, QC, m, ij, sx, B); #c1(FOXP1) c2(NP_001231737) c3(forkhead box protein P1 isoform 3) c4( )c5(87440, 87441) c6( )c7(T, aC, fM, pf, sw, VT, aA, bD, bwX, jh, j, ci, nA, qj, ad, anU, x, yt, acA, zK, aT, LA, cy, Ko, KU, he, hH, ake, ae, rz, d, VX, BO, aW, eH, bwY, eF, t, bN, aUn, rR, yl, QG, eM, au, X, cj, MW, QC, m, ij, sx, B); #c1(FOXP1) c2(NP_001231741) c3(forkhead box protein P1 isoform 5) c4(87442) c5(87443, 87444, 87445, 87446, 87447, 87448) c6( ) c7(T, aC, fM, pf, sw, VT, aA, bD, bwX, jh, j, ci, nA, qj, ad, anU, x, yt, acA, zK, aT, LA, cy, Ko, KU, he, hH, ake, ae, rz, d, VX, BO, aW, eH, bwY, eF, t, bN, aUn, rR, yl, QG, eM, au, X, cj, MW, QC, m, ij, sx, B); #c1(FOXP1) c2(NP_001231742) c3(forkhead box protein P1 isoform 6) c4(87449, 87450, 87451) c5( )c6( )c7(T, aC, fM, pf, sw, VT, aA, bD, bwX, jh, j, ci, nA, qj, ad, anU, x, yt, acA, zK, aT, LA, cy, Ko, KU, he, hH, ake, ae, rz, d, VX, BO, aW, eH, bwY, eF, t, bN, aUn, rR, yl, QG, eM, au, X, cj, MW, QC, m, ij, sx, B); #c1(FOXP1) c2(NP_001231744) c3(forkhead box protein PI isoform 8) c4(87452, 87453) c5(87454, 87455, 87456, 87457, 87458, 87459) c6( )c7(T, aC, fM, pf, sw, VT, aA, bD, bwX, jh, j, ci, nA, qj, ad, anU, x, yt, acA, zK, aT, LA, cy, Ko, KU, he, hH, ake, ae, rz, d, VX, BO, aW, eH, bwY, eF, t, bN, aUn, rR, yl, QG, eM, au, X, cj, MW, QC, m, ij, sx, B); #c1 (FOXP1) c2(NP_001231745) c3(forkhead box protein P1 isoform 1) c4(87460, 87461) c5(87462, 87463, 87464, 87465, 87466, 87467, 87468, 87469, 87470, 87471, 87472, 87473, 87474, 87475, 87476, 87477, 87478, 87479, 87480, 87481, 87482) c6( )c7(T, aC, fM, pf, sw, VT, aA, bD, bwX, jh, j, ci, nA, qj, ad, anU, x, yt, acA, zK, aT, LA, cy, Ko, KU, he, hH, ake, ae, rz, d, VX, BO, aW, eH, bwY, eF, t, bN, aUn, rR, yl, QG, eM, au, X, cj, MW, QC, m, ij, sx, B); #c1(FOXP1) c2(XP_005264799) c3(forkhead box protein P1 isoform X6) c4( )c5(279232, 279233, 279234, 279235, 279236, 279237, 279238, 279239, 279240, 279241, 279242, 279243, 279244, 279245, 279246, 279247) c6( ) c7(T, aC, fM, pf, sw, VT, aA, bD, bwX, jh, j, ci, nA, qj, ad, anU, x, yt, acA, zK, aT, LA, cy, Ko, KU, he, hH, ake, ae, rz, d, VX, BO, aW, eH, bwY, eF, t, bN, aUn, rR, yl, QG, eM, au, X, cj, MW, QC, m, ij, sx, B); #c1(FOXP2) c2(NP_001166237) c3(forkhead box protein PZ isoform V) c4( ) c5(68492, 68493, 68494, 68495, 68496, 68497, 68498, 68499, 68500, 68501, 68502, 68503, 68504, 68505, 68506, 68507, 68508, 68509, 68510, 68511, 68512, 68513, 68514, 68515, 68516, 68517, 68518, 68519, 68520, 68521, 68522, 68523, 68524, 68525, 68526, 68527) c6( )c7(bL, T, iH, ha, VT, bO, fd, n, jh, v, bxb, aR, ad, anU, bxc, aup, bwZ, HJ, WY, qK, aQQ, acg, bK, aQM, K, KU, he, rz, ake, ae, je, LO, Az, fe, aaE, aha, QK, xw, bN, bxa, aUn, iM, abV, Km, yA); #c1(FOXP2) c2(NP_001166238) c3(forkhead box protein P2 isoform VI) c4(68528) c5( )c6( )c7(bL, T, iH, ha, VT, bO, fd, n, jh, v, bxb, aR, ad, anU, bxc, aup, bwZ, HJ, WY, qK, aU, acg, bK, aQM, K, KU, he, rz, ake, ae, je, LO, Az, fe, aaE, aha, QK, xw, bN, bxa, aUn, iM, abV, Km, yA); #c1(FOXP2) c2(NP_055306) c3(forkhead box protein P2 isoform 1) c4(180386, 180387) c5( )c6( )c7(bL, T, iH, ha, VT, bO, fd, n, jh, v, bxb, aR, ad, anU, bxc, aup, bwZ, HJ, WY, qK, aQQ, acg, bK, aQM, K, KU, he, rz, ake, ae, je, LO, Az, fe, aaE, aha, QK, xw, bN, bxa, aUn, iM, abV, Km, yA); #c1(FOXP2) c2(NP_683696) c3(forkhead box protein P2 isoform II) c4(242402) c5( )c6( )c7(bL, T, iH, ha, VT, bO, fd, n, jh, v, bxb, aR, ad, anU, bxc, aup, bwZ, HJ, WY, qK, aQQ, acg, bK, aQM, K, KU, he, rz, ake, ae, je, LO, Az, fe, aaE, aha, QK, xw, bN, bxa, aUn, iM, abV, Km, yA); #c1(FOXP2) c2(NP_683697) c3(forkhead box protein P2 isoform III) c4(242403) c5(242404, 242405, 242406, 242407, 242408, 242409, 242410, 242411, 242412, 242413, 242414, 242415, 242416, 242417, 242418, 242419, 242420, 242421, 242422, 242423, 242424, 242425, 242426, 242427, 242428) c6( )c7(bL, T, iH, ha, VT, bO, fd, n, jh, v, bxb, aR, ad, anU, bxc, aup, bwZ, HJ, WY, qK, aQQ, acg, bK, aQM, K, KU, he, rz, ake, ae, je, LO, Az, fe, aaE, aha, QK, xw, bN, bxa, aUn, iM, abV, Km, yA); #c1(FOXP2) c2(NP_683698) c3(forkhead box protein P2 isoform (V) c4(242429, 242430, 242431, 242432) c5( )c6( )c7(bL, T, iH, ha, VT, bO, fd, n, jh, v, bxb, aR, ad, anU, bxc, aup, bwZ, HJ, WY, qK, aQQ, acg, bK, aQM, K, KU, he, rz, ake, ae, je, LO, Az, fe, aaE, aha, QK, xw, bN, bxa, aUn, iM, abV, Km, yA); #c1(FOXP3) c2(NP_001107849) c3(forkhead box protein P3 isoform b) c4( )c5(43014, 43015, 43016, 43017, 43018, 43019, 43020, 43021, 43022, 43023, 43024) c6( )c7(bM, G, T, bwl, fR, bxe, if, eb, bB, fd, cL, qj, ci, bh, Qp, AW, xE, aE, rw, dV, Ds, rB, K, DY, bhm, bc, cp, mH, YL, akV, aW, az, hN, g, as, eE, bw, mK, iP, yd, eM, Jr, mx, cj, dW, m, eg, fy, cE, abh, akc, aO, NT, fN, n, bwX, aj, ml, qN, qW, cd, ai, Vs, jz, mh, auj, ju, gd, kH, Ne, h, bxi, eV, OM, bxg, aVn, ajV, A, CM, lb, ks, fe, kp, bxh, beP, YF, jT, adz, Wt, dB, at, uz, eo, fL, B, Dw, dA, fG, lH, jx, eX, aA, cz, hz, v, j, ON, C, V, pd, q, J, o, eA, lg, afQ, aT, qG, bK, aFm, ae, hM, kl, bf, xR, nO, oV, Y, fO, Pg, r, aMA, afq, lo, en, t, f, pb, OC, ck, cT, OW, Dy, iM, ig, hc, gX, hr, bxj, Da, yH, gT, bt, zA, aya, hS, Kd, aD, iW, jh, jE, afD, mC, ad, wD, LH, dP, hX, av, F, oz, Dq, ds, qh, hD, JK, ji, bxf, lQ, yF, uQ, d, je, ff, cr, eZ, dN, bxd, af, hp, S, BG, iV, bz, Bv, eU, bE, Lj, anY, z, Km, XO, aEN, lf, ra, y, pC); #c1(FOXP3) c2(NP_054728) c3(forkhead box protein P3 isoform a) c4(177381, 177382) c5(177383, 177384, 177385, 177386, 177387, 177388, 177389, 177390, 177391, 177392, 177393) c6( )c7(bM, G, T, bwU, fR, bxe, if, eb, bB, fd, cL, qj, ci, bh, Qp, AW, xE, aE, rw, dV, Ds, rB, K, DY, bhm, bc, cp, mH, YL, akV, aW, az, hN, g, as, eE, bw, mK, iP, yd, eM, Jr, mx, cj, dW, m, eg, fy, cE, abh, akc, aD, NT, fN, n, bwX, aj, ml, qN, qW, cd, ai, Vs, jz, mh, auj, ju, gd, kH, Ne, h, bxi, eV, OM, bxg, aVn, ajV, A, CM, lb, ks, fe, kp, bxh, beP, YF, jT, adz, Wt, dB, at, uz, eo, fL, B, Dw, dA, fG, lH, jx, eX, aA, cz, hz, v, j, ON, C, V, pd, q, J, o, eA, lg, afQ, aT, qG, bK, aFm, ae, hM, kl, bf, xR, nO, oV, Y, FO, Pg, r, aMA, afq, lo, en, t, f, pb, OC, ck, cT, OW, Dy, iM, ig, hc, gX, hr, bxj, Da, yH, gT, bt, zA, aya, hS, Kd, aD, iW, jh, jE, afD, mC, ad, wD, LH, dP, hX, av, F, oz, Dq, ds, qh, hD, JK, ji, bxf, lQ, yF, uQ, d, je, ff, cr, eZ, dN, bxd, aF, hp, S, BG, iV, bz, Bv, eU, bE, Lj, anY, z, Km, XO, aEN, lf, ra, y, pC); #c1(FOXP3) c2(XP_011542220) c3 (forkhead box protein P3 isoform X4) c4( )c5(340719, 340720) c6( )c7(bM, G, T, bwU, fR, bxe, if, eb, bB, fd, cL, qj, ci, bh, @p, AW, xE, aE, rw, dV, Ds, rB, K, DY, bhm, bc, cp, mH, YL, akV, aW, az, hN, g, as, eE, bw, mK, iP, yd, eM, Jr, mx, cj, dW, m, eg, fy, cE, abh, akc, aO, NT, fN, n, bwX, aj, ml, qN, qW, cd, ai, Vs, jz, mh, auj, ju, gd, kH, Ne, h, bxi, eV, OM, bxg, aVn, ajV, A, CM, lb, ks, fe, kp, bxh, beP, YF, jT, adz, Wt, dB, at, uz, eo, fL, B, Dw, dA, fG, lH, jx, eX, aA, cz, hz, v, j, ON, C, V, pd, q, J, o, eA, lg, afQ, aT, qG, bK, aFm, ae, hM, kl, bf, xR, nO, oV, Y, fO, Pg, r, aMA, afq, lo, en, t, f, pb, OC, ck, cT, OW, Dy, iM, ig, hc, gX, hr, bxj, Da, yH, gT, bt, zA, aya, hS, Kd, aD, iW, jh, jE, afD, mC, ad, wD, LH, dP, hX, av, F, oz, Dq, ds, qh, hD, JK, ji, bxf, lQ, yF, uQ, d, je, ff, cr, eZ, dN, bxd, aF, hp, S, BG, iV, bz, Bv, eU, bE, Lj, anY, z, Km, XO, aEN, lf, ra, y, pC); #c1 (FOXP3) c2(XP_011542221) c3 (forkhead box protein P3 isoform X5) c4(340721) c5(340722, 340723) c6( )c7(bM, G, T, bwl, fR, bxe, if, eb, bB, fd, cL, qj, ci, bh, Qp, AW, xE, aE, rw, dV, Ds, rB, K, DY, bhm, bc, cp, mH, YL, akV, aW, az, hN, g, as, eE, bw, mK, iP, yd, eM, Jr, mx, cj, dW, m, eg, fy, cE, abh, akc, aO, NT, fN, n, bwX, aj, ml, qN, qW, cd, ai, Vs, jz, mh, auj, ju, gd, kH, Ne, h, bxi, eV, OM, bxg, aVn, ajV, A, CM, lb, ks, fe, kp, bxh, beP, YF, jT, adz, Wt, dB, at, uz, eo, fL, B, Dw, dA, fG, lH, jx, eX, aA, cz, hz, v, j, ON, C, V, pd, q, J, o, eA, lg, afQ, aT, qG, bK, aFm, ae, hM, kl, bf, xR, nO, oV, Y, fO, Pg, r, aMA, afq, lo, en, t, f, pb, OC, ck, cT, OW, Dy, iM, ig, hc, gX, hr, bxj, Da, yH, gT, bt, ZA, aya, hS, Kd, aD, iW, jh, jE, afD, mC, ad, wD, LH, dP, hX, av, F, oz, Dq, ds, qh, hD, JK, ji, bxf, lQ, yF, uQ, d, je, ff, cr, eZ, dN, bxd, aF, hp, S, BG, iV, bz, Bv, eU, bE, Lj, anY, z, Km, XO, aEN, lf, ra, y, pC); #c1(FOXP4) c2(NP_001012426) c3(forkhead box protein P4 isoform 1) c4(24987) c5(24988, 24989, 24990, 24991, 24992, 24993, 24994, 24995, 24996, 24997) c6( )c7(T, ae, ad); #c1(FOXP4) c2(NP_001012427) c3(forkhead box protein P4 isoform 3) c4(24998) c5(24999, 25000, 25001, 25002, 25003, 25004) c6( )c7(T, ae, ad); #c1(FOXP4) c2(NP_612466) c3(forkhead box protein P4 isoform 2) c4(236592, 236593) c5(236594, 236595, 236596, 236597, 236598, 236599, 236600, 236601, 236602, 236603, 236604, 236605, 236606) c6( )c7(T, ae, ad); #c1(FOXQ1) c2(NP_150285) c3(forkhead box protein Q1) c4(228051) c5(228052, 228053, 228054, 228055, 228056, 228057, 228058) c6( )c7(XX, T, aC, eM, aW, ff, aT, r, n, adR, aA, dL, m, q, en, g, eA, aF, eL, B); #c1(FOXR1) c2(NP_859072) c3(forkhead box protein RI) c4(255501) c5(255502, 255503, 255504, 255505, 255506, 255507) c6( ) c7(eF, cm, eH); #c1(FOXR2) c2(NP_940853) c3(forkhead box protein R2) c4(259953) c5(259954, 259955, 259956, 259957, 259958, 259959) c6( )c7(jf, aW, ael, hP, aT); #c1(FOXRED1) c2(NP_060017) c3(FAD-dependent oxidoreductase domain-containing protein 1) c4(193666, 193667, 193668) c5(193669, 193670, 193671, 193672, 193673, 193674, 193675, 193676, 193677, 193678, 193679, 193680, 193681) c6( )c7(aM, bxk, bxl, px, kC, ajU); #c1(FOXRED1) c2(XP_006718942) c3(FAD-dependent oxidoreductase domain-containing protein 1 isoform X1) c4( )c5(292114, 292115, 292116, 292117, 292118) c6( )c7(aM, bxk, bxl, px, kC, ajU); #c1(FPGS) c2(NP_001275732) c3(folylpolyglutamate synthase, mitochondrial isoform c precursor) c4(107223, 107224) c5(107225, 107226, 107227, 107228, 107229, 107230, 107231, 107232, 107233, 107234, 107235, 107236) c6( )c7(ng, ck, T, x, fM, lp, bE, iW, eH, cr, r, bB, hn, k, qN, eF, y, q); #c1(FPGS) c2(NP_004948) c3(folylpolyglutamate synthase, mitochondrial isoform a precursor) c4(151398, 151399, 151400, 151401) c5(151402, 151403, 151404, 151405, 151406, 151407, 151408) c6( )c7(ng, ck, T, x, fM, lp, bE, iW, eH, cr, r, bB, hn, k, qN, eF, y, q); #c1(FPGS) c2(XP_005251921) c3(folylpolyglutamate synthase, mitochondrial isoform X2) c4( )c5(271285, 271286, 271287, 271288, 271289, 271290, 271291, 271292, 271293, 271294, 271295, 271296, 271297, 271298, 271299) c6( )c7(ng, ck, T, x, fM, lp, bE, iW, eH, cr, r, bB, hn, k, qN, eF, y, q); #c1(FPGS) c2(XP_011516740) c3(folylpolyglutamate synthase, mitochondrial isoform X1) c4( )c5(310200, 310201, 310202, 310203, 310204) c6( )c7(ng, ck, T, x, fM, lp, bE, iW, eH, cr, r, bB, hn, k, qN, eF, y, q); #c1(FPGS) c2(XP_011516741) c3(folylpolyglutamate synthase, mitochondrial isoform X3) c4( )c5(310205) c6( )c7(ng, ck, T, x, fM, lp, bE, iW, eH, cr, r, bB, hn, k, qN, eF, y, q); #c1(FPRI) c2(NP_001180235) c3(fMet-Leu-Phe receptor) c4(73486) c5(73487, 73488, 73489, 73490, 73491, 73492, 73493, 73494, 73495, 73496) c6( )c7(d, aFe, eZ, aFb, ld, uR, aT, cz, uz, ace, v, h, eA, cP, as, bD, f, Df, Tz, fc); #c1(FPR2) c2(XP_006723183) c3(N-formyl peptide receptor 2 isoform X1) c4(295022) c5(295023, 295024, 295025, 295026, 295027, 295028, 295029, 295030, 295031, 295032) c6( ) c7(bM, T, dD, ea, bkF, bB, eq, ai, eA, afb, dP, aE, ju, adX, aT, h, dl, K, eL, Y, eH, fe, as, eF, t, nM, adZ, bE, wl, m, y, L); #c1(FPR3) c2(NP_002021) c3(N-formyl peptide receptor 3) c4(123366) c5(123367, 123368, 123369, 123370, (23371, 123372) c6( )c7(oe, ey); #c1(FRAS1) c2(NP_001159605) c3(extracellular matrix protein FRAS1 isoform 2 precursor) c4(64433) c5(64434, 64435, 64436, 64437, 64438, 64439, 64440, 64441, 64442, 64443, 64444, 64445, 64446, 64447, 64448, 64449, 64450, 64451, 64452, 64453, 64454, 64455, 64456, 64457, 64458, 64459, 64460, 64461, 64462) c6( )c7(XW, pN, bxn, bxm, bxo, Qt, aMi); #c1(FRAS1) c2(NP_079350) c3(extracellular matrix protein FRAS1 isoform 1 precursor) c4(217794, 217795, 217796, 217797, 217798) c5(217799, 217800, 217801, 217802, 217803, 217804, 217805, 217806, 217807, 217808, 217809, 217810, 217811) c6( )c7(XW, pN, bxn, bxm, bxo, Qt, aMi); #c1(FRAS1) c2(XP_011530572) c3(extracellular matrix protein FRAS1 isoform X3) c4(326493) c5( )c6( )c7(XW, pN, bxn, bxm, bxo, Qt, aMi); #c1(FRAS1) c2(XP_011530573) c3(extracellular matrix protein FRAS1 isoform X4) c4(326494) c5( )c6( )c7(XW, pN, bxn, bxm, bxo, Qt, aMi); #c1(FRAT1) c2(NP_005470) c3(proto-oncogene FRAT1) c4(156435) c5(156436, 156437, 156438, 156439, 156440, 156441) c6( )c7(T, d, aE, eM, il, aW, if, ht, aT, aA, aF, m, qN, hu, g, t, bz, eA); #c1(FRAT2) c2(NP_036215) c3(GSK-3-binding protein FRAT2) c4(172418) c5(172419, 172420, 172421, 172422, 172423, 172424) c6( )c7(T, aF, W, aA); #c1(FREM1) c2(XP_006716792) c3(FRAS1-related extracellular matrix protein 1 isoform X2) c4( )c5(290453, 290454, 290455, 290456, 290457, 290458, 290459, 290460, 290461, 290462, 290463) c6( )c7(bxt, bxs, tL, bxn, De, wk, bxp, bxq, aw, bxr, aMi); #c1(FREM1) c2(XP_011516053) c3(FRAS1-related extracellular matrix protein 1 isoform X1) c4(309243, 309244) c5(309245, 309246, 309247, 309248, 309249, 309250, 309251, 309252, 309253, 309254, 309255, 309256, 309257, 309258, 309259, 309260) c6( )c7(bxt, bxs, tl, bxn, oe, wk, bxp, bxq, aw, bxr, aMi); #c1(FREM1) c2(XP_011516057) c3(FRAS1-related extracellular matrix protein 1 isoform X6) c4(309261) c5( )c6( )c7(bxt, bxs, tL, bxn, oe, wk, bxp, bxq, aw, bxr, aMi); #c1(FREM1) c2(XP_011516060) c3 (FRAS1-related extracellular matrix protein 1 isoform X11) c4(309262) c5(309263) c6( )c7(bxt, bxs, tL, bxn, oe, wk, bxp, bxq, aw, bxr, aMi); #c1(FREM2) c2(NP_997244) c3(FRAS1-related extracellular matrix protein 2 precursor) c4(265293) c5(265294, 265295, 265296, 265297, 265298, 265299) c6( )c7(Qf, bxn, cd, eA, aMi, On); #c1(FREM3) c2(NP_001161707) c3(FRAS1-related extracellular matrix protein 3 precursor) c4( )c5(65834) c6( )c7(bO); #c1(FRG1) c2(NP_004468) c3(protein FRG1) c4(146612, 146613, 146614, 146615) c5(146616, 146617, 146618, 146619, 146620, 146621, 146622, 146623, 146624, 146625, 146626, 146627, 146628, 146629, 146630, 146631, 146632, 146633, 146634, 146635, 146636, 146637, 146638, 146639, 146640, 146641, 146642, 146643) c6( ) c7(asp, Ae, xs, oj, rx, bxu); #c1(FRG2) c2(NP_001005217) c3(protein FRG2 isoform 2) c4(21198) c5(21199, 21200, 21201, 21202, 21203, 21204, 21205, 21206, 21207) c6( )c7(Z, rx, bS, ct); #c1(FRG2) c2(NP_001273749) c3(protein FRG2 isoform 1) c4(105760) c5(105761, 105762, 105763, 105764, 105765, 105766) c6( )c7(Z, rx, bS, ct); #c1(FRK) c2(XP_011533957) c3(tyrosine-protein kinase FRK isoform X1) c4(330159) c5(330160, 330161, 330162, 330163, 330164, 330165) c6( )c7(T, W, af, aW, aT, h, aq, mf, iR, bt, a, B, ad); #c1(FRK) c2(XP_011533958) c3(tyrosine-protein kinase FRK isoform X2) c4(330166) c5(330167, 330168, 330169, 330170, 330171, 330172) c6( ) c7(T, W, af, aW, aT, h, aq, mf, iR, bt, ae, B, ad); #c1(FRMD3) c2(NP_001231888) c3(FERM domain-containing protein 3 isoform 2) c4(87643, 87644) c5(87645, 87646, 87647, 87648, 87649, 87650) c6( )c7(bL, av, A, eR, vy, NI, aj, dy, aY, bz, J); #c1(FRMD3) c2(NP_001231890) c3(FERM domain-containing protein 3 isoform 4) c4(87651) c5(87652, 87653, 87654, 87655, 87656, 87657, 87658, 87659, 87660, 87661, 87662, 87663) c6( )c7(bL, av, A, eR, vy, NI, aj, dy, aY, bz, J); #c1(FRMD3) c2(NP_001231891) c3(FERM domain-containing protein 3 isoform 5) c4(87664) c5(87665, 87666, 87667, 87668, 87669, 87670) c6( )c7(bL, av, A, eR, vy, NI, aj, dy, aY, bz, J); #c1(FRMD3) c2(NP_777598) c3(FERM domain-containing protein 3 isoform 1) c4(251300) c5(251301, 251302, 251303, 251304, 251305, 251306) c6( )c7(bL, av, A, eR, vy, NI, aj, dy, aY, bz, J); #c1(FRMD3) c2(XP_005251939) c3(FERM domain-containing protein 3 isoform X1) c4(271316) c5( )c6( )c7(bL, av, A, eR, vy, NI, aj, dy, aY, bz, J); #c1(FRMD3) c2(XP_011516778) c3(FERM domain-containing protein 3 isoform X2) c4( )c5(310224, 310225, 310226, 310227, 310228, 310229) c6( )c7(bL, av, A, eR, vy, NI, aj, dy, aY, bz, J); #c1(FRMD4A) c2(NP_060497) c3(FERM domain-containing protein 4A) c4(196247) c5(196248, 196249, 196250, 196251, 196252, 196253, 196254, 196255, 196256) c6( )c7(IB, XY); #c1(FRMD4A) c2(XP_005252546) c3(FERM domain-containing protein 4A isoform X9) c4( ) c5(271874) c6( )c7(IB, XY); #c1(FRMD4A) c2(XP_011517845) c3(FERM domain-containing protein 4A isoform X13) c4( )c5(311784) c6( ) c7(IB, XY); #c1(FRMD4B) c2(NP_055938) c3(FERM domain-containing protein 4B) c4(184563) c5(184564, 184565, 184566, 184567, 184568, 184569, 184570) c6( )c7(cc, fq, cd, hp, bX); #c1(FRMD4B) c2(XP_005264780) c3(FERM domain-containing protein 48 isoform X6) c4( )c5(279227, 279228, 279229, 279230, 279231) c6( )c7(cc, fq, cd, hp, bX); #c1(FRMD4B) c2(XP_006713127) c3(FERM domain-containing protein 48 isoform X5) c4(287046) c5(287047) c6( )c7(cc, fq, cd, hp, bX); #c1(FRMD4B) c2(XP_011531828) c3(FERM domain-containing protein 48 isoform X3) c4(327896) c5(327897, 327898, 327899, 327900, 327901) c6( )c7(cc, fq, cd, hp, bX); #c1(FRMD5) c2(NP_001273419) c3(FERM domain-containing protein 5 isoform 3) c4(104859) c5(104860, 104861, 104862, 104863, 104864) c6( ) c7(bz); #c1(FRMD5) c2(NP_116281) c3(FERM domain-containing protein 5 isoform 2) c4(226080) c5(226081, 226082, 226083, 226084, 226085, 226086) c6( )c7(bz); #c1(FRMD5) c2(XP_011520425) c3(FERM domain-containing protein 5 isoform X3) c4( )c5(315316) c6( ) c7(bz); #c1(FRMD6) c2(NP_001253976) c3(FERM domain-containing protein 6 isoform 3) c4( )c5(94343, 94344, 94345, 94346, 94347) c6( )c7(hb, bM, fq, cd); #c1(FRMD6) c2(XP_006720092) c3(FERM domain-containing protein 6 isoform X2) c4(293213, 293214, 293215) c5(293216, 293217, 293218, 293219, 293220, 293221) c6( )c7(hb, bM, fq, cd); #c1(FRMD6) c2(XP_006720093) c3(FERM domain-containing protein 6 isoform X1) c4(293222) c5(293223, 293224, 293225, 293226, 293227, 293228, 293229, 293230, 293231, 293232, 293233, 293234, 293235, 293236, 293237, 293238, 293239) c6( )c7(hb, bM, fq, cd); #c1(FRMD7) c2(NP_919253) c3(FERM domain-containing protein 7) c4(258300) c5(258301, 258302, 258303, 258304, 258305, 258306) c6( )c7(aAd, KE, bxv, aQu, aiM, aSW, NG, bxx, bxw, ol, amA, bna); #c1(FRMPD1) c2(NP_055722) c3(FERM and PDZ domain-containing protein 1) c4(183072, 183073, 183074, 183075) c5(183076, 183077, 183078, 183079, 183080, 183081, 183082, 183083, 183084, 183085, 183086, 183087, 183088, 183089, 183090, 183091, 183092, 183093, 183094, 183095, 183096, 183097, 183098, 183099, 183100, 183101, 183102, 183103) c6( )c7(zP, oe, ac); #c1(FRMPD1) c2(XP_011516108) c3(FERM and PDZ domain-containing protein 1 isoform X3) c4( )c5(309332) c6( )c7(zP, De, ac); #c1(FRMPD2) c2(NP_001018081) c3(FERM and PDZ domain-containing protein 2 isoform 3) c4(27139, 27140) c5(27141, 27142, 27143, 27144, 27145, 27146) c6( )c7(iJ, fN); #c1(FRMPD2) c2(NP_001035977) c3(FERM and PDZ domain-containing protein 2 isoform 4) c4( ) c5(33605, 33606, 33607, 33608, 33609) c6( )c7(iJ, fN); #c1(FRMPD4) c2(NP_055543) c3(FERM and PDZ domain-containing protein 4) c4(181849) c5(181850, 181851, 181852, 181853, 181854, 181855, 181856, 181857, 181858, 181859, 181860, 181861, 181862, 181863, 181864, 181865, 181866, 181867) c6( )c7(oe, an); #c1(FRMPD4) c2(XP_011543915) c3(FERM and PDZ domain-containing protein 4 isoform X2) c4(343105, 343106) c5(343107) c6( )c7(oe, an); #c1(FRRS1L) c2(NP_055149) c3(DOMON domain-containing protein FRRS1L) c4(179456) c5(179457, 179458, 179459, 179460, 179461, 179462) c6( )c7(j); #c1(FRS2) c2(NP_001265286) c3(fibroblast growth factor receptor substrate 2) c4(98127, 98128, 98129, 98130, 98131, 98132, 98133) c5(98134, 98135, 98136, 98137, 98138, 98139, 98140, 98141, 98142, 98143, 98144, 98145, 98146, 98147, 98148, 98149, 98150, 98151, 98152, 98153, 98154, 98155, 98156, 98157, 98158, 98159, 98160, 98161, 98162, 98163, 98164, 98165, 98166, 98167, 98168, 98169, 98170, 98171, 98172, 98173, 98174, 98175, 98/76, 98177, 98178, 98179, 98180, 98181, 98182, 98183, 98184, 98185, 98186, 98187, 98188, 98189, 98190) c6( )c7(bxy, hd, YE, ik, aE, hV, aW, Y, au, aT, cz, ae, ad); #c1(FRS3) c2(XP_011512557) c3(fibroblast growth factor receptor substrate 3 isoform X1) c4(303429, 303430, 303431) c5(303432, 303433, 303434, 303435, 303436, 303437, 303438, 303439, 303440, 303441, 303442, 303443, 303444, 303445, 303446, 303447, 303448, 303449, 303450, 303451, 303452, 303453) c6( )c7(ae, af, ad, eM); #c1(FRY) c2(NP_075463) c3(protein furry homolog) c4(213906, 213907, 213908) c5(213909, 213910, 213911, 213912, 213913, 213914, 213915, 213916, 213917, 213918, 213919, 213920, 213921, 213922, 213923, 213924, 213925, 213926, 213927, 213928, 213929, 213930, 213931, 213932, 213933) c6( )c7(T, aW, aT, Ea, bX, nA); #c1(FRY) c2(XP_006719812) c3(protein furry homolog isoform X1) c4(292973, 292974, 292975, 292976) c5(292977, 292978, 292979, 292980, 292981, 292982, 292983, 292984, 292985, 292986, 292987, 292988, 292989, 292990, 292991, 292992, 292993) c6( )c7(T, aW, aT, Ea, bX, nA); #c1(FRYL) c2(NP_055845) c3(protein furry homolog-like) c4(183875, 183876) c5(183877, 183878, 183879, 183880, 183881, 183882, 183883, 183884, 183885, 183886, 183887, 183888, 183889, 183890, 183891, 183892) c6( )c7(ck, cm, bw, br); #c1(FRYL) c2(XP_011511984) c3(protein furry homolog-like isoform X3) c4(302644) c5(302645, 302646) c6( )c7(ck, cm, bw, br); #c1(FRYL) c2(XP_011511986) c3(protein furry homolog-like isoform X5) c4( )c5(302647, 302648, 302649, 302650, 302651, 302652, 302653, 302654, 302655) c6( )c7(ck, cm, bw, br); #c1(FRZB) c2(NP_001454) c3(secreted frizzled-related protein 3 precursor) c4(117949) c5(117950, 117951, 117952, 117953, 117954, 117955) c6( )c7(gh, T, os, AB, pf, if, aA, fd, oZ, j, fr, qN, ai, ad, cd, q, zw, ib, aT, bav, YT, bA, jf, s, aW, Y, r, aF, bu, aDQ, es, iA, tV, nw, y, atO); #c1 (FSBP) c2(NP_001243070) c3(fibrinogen silencer-binding protein) c4(89729, 89730) c5(89731, 89732, 89733, 89734, 89735, 89736, 89737, 89738, 89739, 89740, 89741, 89742) c6( )c7(T, kl, bE, aZ, bB, uV); #c1(FSCB) c2(NP_115511) c3(fibrous sheath CABYR-binding protein) c4(222997, 222998) c5(222999, 223000, 223001, 223002, 223003, 223004, 223005, 223006, 223007, 223008, 223009, 223010) c6( )c7(bM, ad); #c1(FSCN1) c2(NP_003079) c3(fascin) c4(135032, 135033, 135034) c5(135035, 135036, 135037, 135038, 135039, 135040, 135041, 135042, 135043, 135044, 135045, 135046, 135047, 135048, 135049, 135050, 135051, 135052) c6( )c7(gh, T, os, Mb, Pt, aA, fd, n, oZ, fr, ci, aub, q, amW, cd, ad, eA, afb, jz, F, bqZ, xE, no, aE, dV, aT, h, By, dL, bU, hu, fb, ae, eL, ul, A, je, s, aW, xi, PX, bxz, Y, ev, r, ff, Ej, kp, af, en, g, LV, cM, f, auC, Bv, iM, ql, nB, bE, avZ, X, Hb, y, bt, it, B, LS); #c1(FSCN2) c2(XP_011522894) c3(fascin-2 isoform X2) c4(317373) c5(317374, 317375, 317376, 317377, 317378, 317379) c6( )c7(bxA, alF, xX, W, nb, dA, nw, nC, bxB, VF); #c1(FSCN2) c2(XP_011522895) c3(fascin-2 isoform X1) c4(317380) c5(317381, 317382, 317383, 317384, 317385, 317386) c6( )c7(bxA, alF, xX, W, nb, dA, nw, nC, bxB, VF); #c1(FSD1) c2(NP_077309) c3(fibronectin type III and SPRY domain-containing protein 1) c4(215179) c5(215180, 215181, 215182, 215183, 215184, 215185) c6( )c7(bM, T, bxC, aw, al, aA, bB, n, Fb, j, cd, q, ad, afb, bv, cc, cy, dL, K, ae, eL, hm, d, Qr, nO, Rj, xs, eH, r, aq, aF, g, eF, iR, ik, bz, bw, dR, ck, es, LB, nM, eM, bE, gZ, np, bD, rA, ik, B, hS); #c1(FSD1L) c2(NP_001138785) c3(FSD1-like protein isoform 3) c4(57691) c5(57692, 57693, 57694, 57695, 57696, 57697) c6( )c7(bM, T, bxC, aw, al, aA, bB, n, Fb, j, cd, q, ad, bv, cc, cy, dL, K, ae, eL, hm, d, Br, nO, Rj, xs, eH, r, aq, aF, g, eF, iR, iK, bz, bw, dR, ck, es, nM, eM, bE, gZ, np, bD, rA, ik, B, hS); #c1(FSD1L) c2(NP_001274120) c3(FSD1-like protein isoform 4) c4(106054) c5(106055) c6( )c7(bM, T, bxC, aw, al, aA, bB, n, Fb, j, cd, q, ad, bv, cc, cy, dL, K, ae, eL, hm, d, Qr, nO, Rj, xs, eH, r, aq, aF, g, eF, iR, iK, bz, bw, dR, ck, es, nM, eM, bE, gZ, np, bD, rA, ik, B, hS); #c1(FSD1L) c2(NP_001274121) c3(FSD1-like protein isoform 5) c4(106056) c5(106057, 106058, 106059, 106060, 106061) c6( )c7(bM, T, bxC, aw, al, aA, bB, n, Fb, j, cd, q, ad, bv, cc, cy, dL, K, ae, eL, hm, d, Qr, nO, Rj, xs, eH, r, aq, aF, g, eF, iR, ik, bz, bw, dR, ck, es, nM, eM, bE, gZ, np, bD, rA, ik, B, hS); #c1(FSD1L) c2(NP_114125) c3(FSD1-like protein isoform 2) c4(222271) c5(222272, 222273, 222274, 222275, 222276, 222277) c6( )c7(bM, T, bxC, aw, al, aA, bB, n, Fb, j, cd, q, ad, bv, cc, cy, dL, K, ae, eL, hm, d, Qr, nO, Rj, xs, eH, r, aq, aF, g, eF, iR, iK, bz, bw, dR, ck, es, nM, eM, bE, gZ, np, bD, rA, ik, B, hS); #c1(FSD1L) c2(XP_005252317) c3(FSD1-like protein isoform X6) c4( )c5(271655, 271656, 271657, 271658, 271659) c6( )c7(bM, T, bxC, aw, al, aA, bB, n, Fb, j, cd, q, ad, bv, cc, cy, dL, K, ae, eL, hm, d, dr, nO, Rj, xs, eH, r, aq, aF, g, eF, iR, ik, bz, bw, dR, ck, es, nM, eM, bE, gZ, np, bD, rA, ik, B, hS); #c1(FSD1L) c2(XP_011517380) c3(FSD1-like protein isoform X3) c4( )c5(311099) c6( )c7(bM, T, bxC, aw, al, aA, bB, n, Fb, j, cd, q, ad, bv, cc, cy, dL, K, ae, eL, hm, d, Ar, nO, Rj, xs, eH, r, aq, aF, g, eF, iR, iK, bz, bw, dR, ck, es, nM, eM, bE, gZ, np, bD, rA, ik, B, hS); #c1(FSD2) c2(NP_001007123) c3(fibronectin type III and SPRY domain-containing protein 2 isoform a) c4(22506, 22507) c5(22508, 22509, 22510, 22511, 22512, 22513) c6( )c7(dY); #c1(FSD2) c2(XP_011519538) c3(fibronectin type III and SPRY domain-containing protein 2 isoform X3) c4( )c5(314191, 314192, 314193, 314194, 314195, 314196) c6( )c7(dY); #c1(FSHB) c2(NP_001018090) c3 (follitropin subunit beta precursor) c4(27194, 27195) c5(27196, 27197, 27198, 27199, 27200, 27201, 27202, 27203, 27204, 27205, 27206, 27207, 27208, 27209, 27210, 27211, 27212, 27213, 27214, 27215, 27216, 27217, 27218, 27219, 27220, 27221, 27222, 27223, 27224, 27225, 27226, 27227, 27228, 27229, 27230, 27231, 27232, 27233, 27234, 27235, 27236, 27237, 27238, 27239, 27240, 27241, 27242, 27243, 27244, 27245, 27246, 27247, 27248, 27249, 27250, 27251, 27252, 27253, 27254, 27255, 27256, 27257, 27258, 27259, 27260, 27261, 27262, 27263, 27264, 27265, 27266, 27267, 27268, 27269, 27270, 27271, 27272, 27273, 27274, 27275, 27276, 27277, 27278, 27279, 27280, 27281, 27282, 27283, 27284, 27285, 27286, 27287, 27288, 27289, 27290, 27291, 27292, 27293, 27294, 27295, 27296, 27297, 27298, 27299, 27300, 27301, 27302) c6(27303, 27304, 27305, 27306, 27307, 27308, 27309) c7(bxD, yo, kl, gU, ND, il, aeS, aT, vH, m, nA, er, ad, bN, UE); #c1(FSHR) c2(NP_000136) c3(follicle-stimulating hormone receptor isoform 1 precursor) c4(2918, 2919) c5(2920, 2921, 2922, 2923, 2924, 2925, 2926) c6( )c7(T, Ut, gU, UH, wu, aZ, aMv, cz, hz, bS, ur, ct, aTS, ci, bxH, uu, v, Ul, ad, dP, il, BJ, avL, zf, auB, aT, vH, UG, wm, uV, af, bA, yo, aev, kl, ZE, ND, Lf, asQ, aeS, eZ, avJ, bxF, dq, Hu, baZ, bu, Z, t, uC, zZ, bfw, qa, bxE, sn, AV, afc, Uv, bxl, KZ, CZ, bxG, m, Lb, gT); #c1(FSHR) c2(NP_852111) c3(follicle-stimulating hormone receptor isoform 2 precursor) c4(254853) c5(254854, 254855, 254856, 254857, 254858, 254859, 254860, 254861, 254862, 254863, 254864, 254865) c6( )c7(T, Ut, gU, UH, wu, aZ, aMv, cz, hz, bS, ur, ct, aTS, ci, bxH, uu, v, Ul, ad, dP, il, BJ, avL, zf, auB, aT, vH, UG, wm, uV, af, bA, yo, aev, kl, ZE, ND, Lf, asQ, aeS, eZ, avJ, bxF, dq, Hu, baZ, bu, Z, t, uC, zZ, bfw, qa, bxE, sn, AV, afc, Uv, bxl, KZ, CZ, bxG, m, Lb, gT); #c1(FSHR) c2(XP_011531035) c3(follicle-stimulating hormone receptor isoform X1) c4(326998) c5( )c6( ) c7(T, Ut, gU, UH, wu, aZ, aMv, cz, hz, bS, ur, ct, aTS, ci, bxH, uu, v, Ul, ad, dP, il, BJ, avL, zf, auB, aT, vH, UG, wm, uV, af, bA, yo, aev, kl, ZE, ND, Lf, asQ, aeS, eZ, avJ, bxF, dq, Hu, baZ, bu, Z, t, uC, zZ, bfw, qa, bxE, sn, AV, afc, Uv, bxl, KZ, CZ, bxG, m, Lb, gT); #c1(FSHR) c2(XP_011531038) c3(follicle-stimulating hormone receptor isoform X3) c4( )c5(326999, 327000, 327001, 327002, 327003) c6( )c7(T, Ut, gU, UH, wu, aZ, aMv, cz, hz, bS, ur, ct, aTS, ci, bxH, uu, v, Ul, ad, dP, il, BJ, avL, zf, auB, aT, vH, UG, wm, uV, af, bA, yo, aev, kl, ZE, ND, Lf, asQ, aeS, eZ, avJ, bxF, dq, Hu, baZ, bu, Z, t, uC, zZ, bfw, qa, bxE, sn, AV, afc, Uv, bxl, KZ, CZ, bxG, m, Lb, gT); #c1(FSIP1) c2(XP_011519608) c3(fibrous sheath-interacting protein 1 isoform X1) c4(314263) c5(314264, 314265, 314266, 314267, 314268, 314269, 314270, 314271, 314272, 314273, 314274) c6( )c7(bM, aqg, aW, aT, dL, j, nA, bu, H, eL, af, xt, L); #c1(FSIPI) c2(XP_011519613) c3(fibrous sheath-interacting protein 1 isoform X6) c4(314275) c5( )c6( )c7(bM, aqg, aW, aT, dL, j, nA, bu, H, eL, af, xt, L); #c1(FST) c2(NP_006341) c3(fallistatin isoform FST317 precursor) c4(164/68, 164169) c5(164/70, 164171, 164/72, 164/73, 164174, 164175) c6( )c7(T, dD, aFG, wu, aZ, ea, pl, cV, aA, eb, oY, v, eq, ci, C, V, cd, ad, Vs, dP, aHF, ft, rw, BJ, rS, aT, dL, bl, wm, Dz, ji, ae, eL, yo, kl, s, qt, aW, Y, ff, xs, uY, aq, zC, en, bu, dw, ajz, t, zE, Px, bxJ, cl, cm, sS, AM, hL, hR, Zn, Da, m, lC, B); #c1(FST) c2(NP_037541) c3(follistatin isoform FST344 precursor) c4(176105, 176106) c5(176107, 176108, 176109, 176110, 176111, 176112, 176113, 176114, 176115, 176116, 176117, 176118, 176119, 176120, 176121, 176122, 176123, 176124, 176125, 176126, 176127, 176128, 176129, 176130, 176131, 176132, 176133, 176134, 176135, 176136, 176137, 176138, 176139, 176140, 176141, 176142, 176143, 176144, 176145, 176146, 176147, 176148, 176149, 176150, 176151, 176152, 176153, 176154, 176155, 176156, 176157, 176158, 176159, 176160, 176161, 176162, 176163, 176164, 176165, 176166, 176167, 176168, 176169, 176170, 176171, 176172, 176173, 176174, 176175, 176176, 176177, 176178, 176179, 176180, 176181, 176182, 176183, 176184, 176185, 176186, 176187, 176188, 176189, 176190, 176191, 176192, 176193, 176194, 176195, 176196, 176197, 176198, 176199, 176200, 176201, 176202, 176203, 176204, 176205, 176206, 176207, 176208, 176209, 176210, 176211, 176212, 176213, 176214, 176215, 176216, 176217, 176218, 176219, 176220, 176221, 176222, 176223, 176224, 176225, 176226, 176227, 176228, 176229, 176230, 176231, 176232, 176233, 176234, 176235, 176236, 176237, 176238, 176239, 176240, 176241, 176242, 176243, 176244, 176245, 176246, 176247, 176248, 176249, 176250, 176251, 176252, 176253, 176254, 176255, 176256) c6(176257) c7(T, dD, aFG, wu, aZ, ea, pl, cV, aA, eb, oY, v, eq, ci, C, V, cd, ad, Vs, dP, aHF, ft, rw, BJ, rS, aT, dL, bl, wm, Dz, ji, ae, eL, yo, kl, s, qt, aW, Y, ff, xs, uY, aq, zC, en, bu, dw, ajz, t, zE, Px, bxJ, cl, cm, sS, AM, hL, hR, Zn, Da, m, lC, B); #c1(FSTL1) c2(NP_009016) c3(follistatin-related protein 1 precursor) c4(170260, 170261, 170262) c5(170263, 170264, 170265, 170266, 170267, 170268, 170269, 170270, 170271, 170272, 170273, 170274, 170275, 170276, 170277, 170278, 170279, 170280) c6( )c7(T, os, bxK, aw, oZ, ai, cd, ad, xE, aE, ws, h, cy, ae, B, cl, t, bz, eM, hL, E, m, y, iu); #c1(FSTL3) c2(NP_005851) c3(follistatin-related protein 3 precursor) c4(159874) c5(159875, 159876, 159877, 159878, 159879, 159880, 159881, 159882) c6( )c7(T, sN, aW, fc, pQ, aT, eb, aq, cP, y, cd); #c1(FSTL4) c2(NP_055897) c3(follistatin-related protein 4 precursor) c4(184288) c5(184289, 184290, 184291, 184292, 184293, 184294, 184295, 184296, 184297, 184298, 184299, 184300, 184301, 184302, 184303, 184304) c6(184305, 184306, 184307, 184308) c7(bM, A, ac, e, aw, cz, alv, j, eu); #c1(FSTL4) c2(XP_011541588) c3(fallistatin-related protein 4 isoform X4) c4(340132) c5( )c6( )c7(bM, A, ac, e, aw, cz, alv, j, eu); #c1(FSTL5) c2(NP_001121899) c3(follistatin-related protein 5 isoform b precursor) c4(46286) c5(46287, 46288, 46289, 46290, 46291, 46292, 46293, 46294, 46295, 46296, 46297, 46298, 46299, 46300) c6(46301, 46302, 46303, 46304) c7(jf, oe, ad); #c1(FSTL5) c2(NP_001121900) c3(follistatin-related protein 5 isoform c precursor) c4(46305) c5(46306, 46307, 46308, 46309, 46310, 46311, 46312, 46313) c6(46314, 46315) c7(jf, oe, ad); #c1(FSTL5) c2(NP_064501) c3(follistatin-related protein 5 isoform a precursor) c4(202636) c5(202637, 202638, 202639, 202640, 202641, 202642, 202643, 202644) c6(202645, 202646) c7(jf, oe, ad); #c1(FTCD) c2(NP_996848) c3(formimidoyltransferase-cyclodeaminase) c4(264772, 264773) c5(264774, 264775, 264776, 264777, 264778, 264779, 264780, 264781) c6( )c7(Na, af, lv, lp, Nc, bh, pk, bxL, B); #c1(FTCD) c2(XP_006724025) c3(formimidoyltransferase-cyclodeaminase isoform X2) c4( )c5(295623, 295624, 295625, 295626, 295627, 295628, 295629, 295630, 295631, 295632) c6( )c7(Na, af, lv, lp, Nc, bh, pk, bxL, B); #c1(FTCD) c2(XP_011527737) c3(formimidoyltransferase-cyclodeaminase isoform X3) c4( )c5(322703) c6( )c7(Na, af, lv, lp, Nc, bh, pk, bxL, B); #c1 (FTCD) c2(XP_011527739) c3(formimidoyltransferase-cyclodeaminase isoform X5) c4( )c5(322704, 322705) c6( )c7(Na, af, lv, lp, Nc, bh, pk, bxL, B); #c1(FTCD) c2(XP_011527740) c3(formimidoyltransferase-cyclodeaminase isoform X6) c4(322706) c5(322707, 322708, 322709, 322710, 322711, 322712, 322713, 322714, 322715, 322716, 322717) c6( )c7(Na, af, lv, lp, Nc, bh, pk, bxL, B); #c1(FTCD) c2(XP_011527742) c3 (formimidoyltransferase-cyclodeaminase isoform X8) c4( )c5(322718, 322719, 322720, 322721, 322722, 322723, 322724, 322725) c6( )c7(Na, af, lv, lp, Nc, bh, pk, bxL, B); #c1(FTH1) c2(NP_002023) c3(ferritin heavy chain) c4(123373, 123374, 123375) c5(123376, 123377, 123378, 123379, 123380, 123381, 123382, 123383, 123384, 123385, 123386, 123387, 123388, 123389, 123390, 123391, 123392, 123393) c6( )c7(mR, fR, ec, yt, pf, ld, U, r, yy, aeu, lo, cd, jh, ct, q, an, Jx, bxM, eA, B); #c1(FTL) c2(NP_000137) c3(ferritin light chain) c4(2927, 2928) c5(2929, 2930, 2931, 2932, 2933, 2934, 2935, 2936, 2937, 2938, 2939, 2940) c6( )c7(fR, aWG, fF, v, ct, hX, axa, yt, oW, aT, aU, K, an, aUV, aUd, cp, Pw, ZH, CN, aW, Y, eH, bcD, aAm, gl, yy, fe, eF, xw, dr, bxN, aeb, aUW, wk, U, y); #c1(FTMT) c2(NP_803431) c3(ferritin, mitochondrial precursor) c4(252629) c5(252630, 252631, 252632, 252633, 252634, 252635) c6( )c7(W, kA, kz, oW, bw, jy, K, an, cm, br, wT); #c1(FTO) c2(NP_001073901) c3(alpha-ketoglutarate-dependent dioxygenase FTO) c4(36371, 36372, 36373, 36374) c5(36375, 36376, 36377, 36378, 36379, 36380, 36381, 36382, 36383, 36384, 36385, 36386, 36387, 36388, 36389, 36390, 36391, 36392, 36393, 36394, 36395, 36396, 36397, 36398, 36399, 36400, 36401, 36402, 36403, 36404, 36405, 36406, 36407, 36408, 36409, 36410, 36411) c6( )c7(bL, bxO, T, jv, dD, bM, eR, aoq, aZ, ea, e, aw, qQ, aOH, cz, bO, fg, cQ, bS, j, ct, afE, aj, hy, v, eq, aR, J, ad, hX, fU, aPW, av, aE, iX, aql, aT, h, qM, he, ae, af, bA, kl, aW, Y, Mz, dq, aq, as, cl, Z, cm, bz, fq, qa, es, pO, cA, bxP, mg, hL, at, mf, lS, ei, B); #c1(FTO) c2(XP_011521615) c3(alpha-ketoglutarate-dependent dioxygenase FTO isoform X1) c4(3164/7) c5(316418) c6( )c7(bL, bxO, T, jv, dD, bM, eR, aoq, aZ, ea, e, aw, qQ, aOH, cz, bO, fg, cd, bS, j, ct, afE, aj, hy, v, eq, aR, J, ad, hX, fU, aPW, av, aE, iX, aql, aT, h, qM, he, ae, af, bA, kl, aW, Y, Mz, dq, aq, as, cl, Z, cm, bz, fq, qa, es, pO, cA, bxP, mg, hL, at, mf, lS, ei, B); #c1(FTSJ1) c2(NP_001269086) c3(putative tRNA (cytidine(32)/guanosine(34)-2′-0)-methyltransferase isoform c) c4( )c5(100396, 100397, 100398, 100399, 100400, 100401) c6( ) c7(nf, t, nX, nA, bxQ); #c1(FTSJ1) c2(NP_036412) c3(putative tRNA (cytidine(32)/guanosine(34)-2′-0)-methyltransferase isoform a) c4(173643, 173644) c5(173645, 173646, 173647, 173648, 173649, 173650) c6( )c7(nf, t, nX, nA, bxQ); #c1(FTSJ1) c2(NP_803188) c3(putative tRNA (cytidine(32)/guanosine(34)-2′-0)-methyltransferase isoform b) c4(252554) c5(252555, 252556, 252557, 252558, 252559, 252560, 252561, 252562, 252563, 252564, 252565, 252566, 252567, 252568, 252569, 252570, 252571, 252572) c6( )c7(nf, t, nX, nA, bxQ); #c1(FTSJ2) c2(NP_037525) c3(putative ribosomal RNA methyltransferase 2) c4(176012, 176013) c5(176014, 176015, 176016, 176017, 176018, 176019, 176020, 176021, 176022, 176023, 176024, 176025) c6( )c7(bv, T, iu, eM); #c1(FTSJ3) c2(NP_060117) c3(pre-rRNA processing protein FTSJ3) c4(194241) c5(194242, 194243, 194244, 194245, 194246, 194247) c6( )c7(t); #c1(FUBP1) c2(NP_001290362) c3(far upstream element-binding protein 1 isoform 1) c4( )c5(115319, 115320, 115321, 115322, 115323) c6( )c7(Qi, dB, aq, eA, as, an, iR, Fc, B); #c1(FUBP1) c2(XP_011540693) c3(far upstream element-binding protein 1 isoform X1) c4(339244) c5( )c6( ) c7(Qi, dB, aq, eA, as, an, iR, Fc, B); #c1(FUBP1) c2(XP_011540694) c3(far upstream element-binding protein 1 isoform X2) c4(339245) c5(339246, 339247, 339248, 339249, 339250, 339251) c6( )c7(Qi, dB, aq, eA, as, an, iR, Fc, B); #c1(FUBP1) c2(XP_011540695) c3(far upstream element-binding protein 1 isoform X3) c4(339252, 339253) c5( )c6( )c7(Di, dB, aq, eA, as, an, iR, Fc, B); #c1(FUBP1) c2(XP_011540696) c3(far upstream element-binding protein 1 isoform X4) c4(339254, 339255) c5(339256, 339257, 339258, 339259, 339260, 339261, 339262, 339263, 339264, 339265, 339266, 339267) c6( )c7(Qi, dB, aq, eA, as, an, iR, Fc, B); #c1(FUBP1) c2(XP_011540697) c3(far upstream element-binding protein 1 isoform X5) c4( )c5(339268) c6( )c7(Di, dB, aq, eA, as, an, iR, Fc, B); #c1(FUBP3) c2(NP_003925) c3(far upstream element-binding protein 3) c4(142225, 142226) c5(142227, 142228, 142229, 142230, 142231, 142232) c6( )c7(B); #c1(FUBP3) c2(XP_0067 (7377) c3 (far upstream element-binding protein 3 isoform X7) c4( )c5(291041) c6( ) c7(B); #c1(FUBP3) c2(XP_011517473) c3(far upstream element-binding protein 3 isoform X1) c4(311205) c5( )c6( )c7(B); #c1(FUBP3) c2(XP_011517474) c3(far upstream element-binding protein 3 isoform X4) c4( )c5(311206, 311207, 311208, 311209, 311210, 311211, 311212, 311213, 311214, 311215, 311216, 311217) c6( )c7(B); #c1(FUCA1) c2(XP_011539469) c3(tissue alpha-L-fucosidase isoform X2) c4( )c5(337672, 337673, 337674, 337675, 337676) c6( )c7(h, bxR, Lq, m, vy); #c1(FUCA2) c2(NP_114409) c3(plasma alpha-L-fucosidase precursor) c4(222608, 222609, 222610) c5(222611, 222612, 222613, 222614, 222615, 222616, 222617, 222618, 222619, 222620, 222621, 222622, 222623, 222624, 222625, 222626, 222627, 222628, 222629, 222630, 222631, 222632, 222633, 222634) c6( )c7(beZ); #c1(FUNDC1) c2(NP_776155) c3(FUN14 domain-containing protein 1) c4(250870) c5(250871, 250872, 250873, 250874, 250875, 250876) c6( )c7(na); #c1(FUNDC2) c2(NP_076423) c3(FUN14 domain-containing protein 2) c4(214119) c5(214120, 214121, 214122, 214123, 214124, 214125) c6( ) c7(qh, B); #c1(FURIN) c2(NP_001276752) c3(furin preproprotein) c4(108350, 108351, 108352) c5(108353, 108354, 108355, 108356, 108357, 108358, 108359, 108360, 108361, 108362, 108363, 108364, 108365, 108366, 108367, 108368, 108369, 108370, 108371) c6(108372) c7(dD, ea, oe, al, cz, hy, eq, jE, ci, zP, q, eA, hX, aT, h, ox, bq, zQ, il, oo, ff, r, en, eM, aS, ao); #c1(FUS) c2(NP_001164408) c3(RNA-binding protein FUS isoform 3) c4( )c5(66928, 66929, 66930, 66931, 66932, 66933, 66934, 66935, 66936, 66937, 66938, 66939) c6( )c7(bL, T, aGn, gU, YU, hV, Ol, bxU, bB, TM, Vd, v, bxV, PJ, QG, ally, aR, ad, av, ft, Gt, di, WY, aT, kf, vH, dz, K, hu, bxT, ae, dx, eG, acR, ND, aW, CT, aaE, cm, bz, QA, dr, iN, ck, rR, RV, bxW, c, bb, Kv, Qu, eM, dJ, au, bxS, U, iT, k, lM, bt); #c1(FUS) c2(NP_004951) c3(RNA-binding protein FUS isoform 1) c4(151418) c5( )c6( )c7(bL, T, aGn, gU, YU, hV, Ol, bxU, bB, TM, Vd, v, bxV, PJ, QG, aHy, aR, ad, av, ft, Gt, di, WY, aT, kf, vH, dz, K, hu, bxT, ae, dx, eG, acR, ND, aW, CT, aaE, cm, bz, QA, dr, iN, ck, rR, RV, bxW, c, bb, Kv, Qu, eM, dJ, au, bxS, U, iT, k, lM, bt); #c1(FUS) c2(XP_011544083) c3(RNA-binding protein FUS isoform X1) c4(343258, 343259) c5( )c6( )c7(bL, T, aGn, gU, YU, hV, Ol, bxU, bB, TM, Vd, v, bxV, PJ, QG, aHy, aR, ad, av, ft, Gt, di, WY, aT, kf, vH, dz, K, hu, bxT, ae, dx, eG, acR, ND, aW, CT, aaE, cm, bz, QA, dr, iN, ck, rR, RV, bxW, c, bb, Kv, Qu, eM, dJ, au, bxS, U, iT, k, lM, bt); #c1(FUS) c2(XP_011544084) c3(RNA-binding protein FUS isoform X2) c4( ) c5(343260, 343261, 343262, 343263, 343264, 343265) c6( )c7(bL, T, aGn, gU, YU, hV, Ol, bxU, bB, TM, Vd, v, bxV, PJ, QG, aHy, aR, ad, av, ft, Gt, di, WY, aT, kf, vH, dz, K, hu, bxT, ae, dx, eG, acR, ND, aW, CT, aaE, cm, bz, QA, dr, iN, ck, rR, RV, bxW, c, bb, Kv, Qu, eM, dJ, au, bxS, U, iT, k, lM, bt); #c1(FUT1) c2(NP_000139) c3(galactoside 2-alpha-L-fucosyltransferase 1) c4( )c5(2941, 2942, 2943, 2944, 2945, 2946, 2947, 2948, 2949, 2950, 2951) c6( )c7(Cx, T, os, dA, al, jh, aA, n, oY, oZ, oH, ci, pz, aR, ad, oT, lE, aE, qh, Co, h, dL, K, jj, an, ae, eL, B, ff, hl, aF, BG, en, jg, t, btO, bz, KM, dr, iK, bw, ck, gk, il, ac, bE, hk, CO, aEG, Lq, m, be, adg, bt, fh, X); #c1(FUT1) c2(XP_006723190) c3(galactoside 2-alpha-L-fucosyltransferase 1 isoform X1) c4(295033, 295034) c5(295035) c6( )c7(Cx, T, os, dA, al, jh, aA, n, oY, oZ, oH, ci, pz, aR, ad, oT, lE, aE, qh, Co, h, dL, K, jj, an, ae, eL, B, ff, hl, aF, BG, en, jg, t, btO, bz, KM, dr, ik, bw, ck, gk, il, ac, bE, hk, CO, aEG, Lq, m, be, adg, bt, fh, X); #c1(FUT10) c2(NP_116053) c3(alpha-(1,3)-fucosyltransferase 10) c4(225287) c5(225288, 225289, 225290, 225291, 225292, 225293, 225294, 225295, 225296, 225297, 225298, 225299) c6( )c7(fq); #c1(FUT10) c2(XP_011542978) c3(alpha-(1,3)-fucosyltransferase 10 isoform X1) c4(341675) c5( )c6( )c7(fq); #c1(FUT10) c2(XP_011542980) c3(alpha-(1,3)-fucosyltransferase 10 isoform X3) c4( )c5(341676, 341677) c6( )c7(fq); #c1(FUT10) c2(XP_011542982) c3(alpha-(1,3)-fucosyltransferase 10 isoform X5) c4(341678) c5(341679, 341680, 341681, 341682, 341683, 341684) c6( )c7(fq); #c1(FUT10) c2(XP_011542983) c3(alpha-(1,3)-fucosyltransferase 10 isoform X6) c4(341685) c5( )c6( )c7(fq); #c1(FUT11) c2(NP_001271123) c3(alpha-(1,3)-fucosyltransferase 1l isoform 2 precursor) c4(103431) c5(103432, 103433, 103434, 103435, 103436, 103437) c6( )c7(py); #c1(FUT11) c2(NP_775811) c3(alpha-(1,3)-fucosyltransferase Il isoform 1 precursor) c4(250389) c5(250390, 250391, 250392, 250393, 250394, 250395) c6( )c7(py); #c1(FUT2) c2(NP_001091107) c3(galactoside 2-alpha-L-fucosyltransferase 2) c4(38507, 38508) c5(38509, 38510, 38511, 38512, 38513, 38514, 38515, 38516, 38517, 38518, 38519, 38520, 38521, 38522, 38523, 38524) c6( )c7(bM, T, Ht, pF, FR, LX, aD, bxY, aA, bB, mN, bS, ct, ad, jz, aE, bxZ, oW, aT, hD, h, dL, K, an, ao, A, az, yy, hN, lo, aF, EZ, Z, t, bya, bxx, bE, at, CZ, m, jM, y, B, LT); #c1(FUT3) c2(XP_011526169) c3(galactoside 3(4)-L-fucosyltransferase isoform X1) c4(320452) c5(320453, 320454, 320455, 320456, 320457, 320458, 320459, 320460, 320461, 320462, 320463, 320464, 320465, 320466, 320467, 320468, 320469, 320470) c6( )c7(bM, T, aC, pF, fR, e, aA, v, ci, fw, q, jz, aE, gd, aT, aa, d, Y, az, r, af, aB, bz, ck, bE, cS, ao); #c1(FUT4) c2(NP_002024) c3(alpha-(1,3)-fucosyltransferase 4) c4(123394) c5(123395, 123396, 123397, 123398, 123399, 123400, 123401) c6( )c7(T, byb, hV, jh, bs, aA, n, bB, Hn, ci, q, br, ad, Nh, eA, av, x, aE, bud, kH, aT, ey, ox, NM, ae, il, jf, d, je, s, il, Cw, aW, Y, ev, az, xR, ff, r, af, en, g, bw, bz, ck, bF, iM, eM, dB, bE, AM, gX, PV, k, pw, bt, it, B); #c1(FUT5) c2(NP_002025) c3(alpha-(1,3)-fucosyltransferase 5) c4(123402) c5(123403, 123404, 123405, 123406, 123407, 123408, 123409, 123410, 123411, 123412) c6( )c7(iu, bE, Y, aA, bB, aF, ci, bz, B, ad); #c1(FUT6) c2(NP_001035791) c3(alpha-(1,3)-fucosyltransferase 6) c4(33071, 33072, 33073, 33074, 33075) c5(33076, 33077, 33078, 33079, 33080, 33081, 33082, 33083, 33084, 33085, 33086, 33087, 33088, 33089, 33090, 33091, 33092, 33093) c6( )c7(ae, T, iu, B, bE, byc, gd, r, aA, bB, aq, aF, ci, bX, bz, q, ad); #c1(FUTG) c2(XP_005259584) c3(alpha-(1,3)-fucosyltransferase 6 isoform X2) c4(276151, 276152) c5(276153, 276154, 276155, 276156, 276157, 276158, 276159, 276160, 276161, 276162, 276163, 276164, 276165, 276166, 276167, 276168, 276169, 276170, 276171, 276172, 276173, 276174, 276175, 276176, 276177, 276178, 276179, 276180, 276181, 276182, 276183, 276184, 276185, 276186, 276187, 276188) c6( )c7(ae, T, iu, B, bE, byc, gd, r, aA, bB, aq, aF, ci, bX, bz, q, ad); #c1(FUT7) c2(NP_004470) c3(alpha-(1,3)-fucosyltransferase 7) c4(146644) c5(146645, 146646, 146647, 146648, 146649, 146650) c6( )c7(T, kl, ac, bP, bE, bB, cL, dL, bz, B, o); #c1(FUT8) c2(NP_004471) c3(alpha-(1,6)-fucosyltransferase isoform b) c4( )c5(146651, 146652, 146653, 146654, 146655) c6( )c7(T, db, n, aq, K, Hb, ci, ae, B, gF, ad, asw); #c1(FUT8) c2(XP_011534915) c3(alpha-(1,6)-fucosyltransferase isoform X1) c4(331428, 331429, 331430, 331431, 331432, 331433, 331434, 331435, 331436) c5(331437, 331438, 331439, 331440, 331441, 331442, 331443, 331444, 331445, 331446, 331447, 331448, 331449, 331450, 331451, 331452, 331453, 331454, 331455, 331456, 331457, 331458, 331459, 331460, 331461, 331462, 331463, 331464, 331465, 331466, 331467, 331468, 331469, 331470, 331471, 331472, 331473, 331474, 331475, 331476, 331477, 331478, 331479, 331480, 331481, 331482, 331483, 331484, 331485, 331486, 331487) c6( )c7(T, db, n, aq, K, Hb, ci, a, B, gF, ad, asw); #c1(FUT8) c2(XP_011534916) c3(alpha-(1,6)-fucosyltransferase isoform X2) c4( )c5(331488, 331489, 331490, 331491, 331492, 331493) c6( )c7(T, db, n, aq, K, Hb, ci, ae, B, gF, ad, asw); #c1(FUT9) c2(XP_011533687) c3(alpha-(1,3)-fucosyltransferase 9 isoform X1) c4(329742) c5(329743, 329744, 329745, 329746, 329747, 329748) c6( )c7(oe, j); #c1(FUZ) c2(NP_001165408) c3(protein fuzzy homolog isoform 2) c4(67655) c5(67656, 67657, 67658, 67659, 67660, 67661) c6( )c7(ng, HF, BU, hl, lp, aX, mg, n, HL, byd, v, K, pw, hK, Gc, Bp, lU); #c1(FUZ) c2(NP_079405) c3(protein fuzzy homolog isoform 1) c4(217938, 217939) c5(217940, 217941, 217942, 217943, 217944, 217945, 217946, 217947, 217948, 217949, 217950, 217951) c6( )c7(ng, HF, BU, hl, lp, aX, mg, n, HL, byd, v, K, pw, hK, Gc, Bp, lU); #c1(FUZ) c2(XP_011525646) c3(protein fuzzy homolog isoform X5) c4(320048) c5(320049, 320050, 320051, 320052, 320053, 320054) c6( )c7(ng, HF, BU, hl, lp, aX, mg, n, HL, byd, v, K, pw, hK, Gc, Bp, lU); #c1(FXN) c2(NP_000135) c3(frataxin, mitochondrial isoform 1 preproprotein) c4(2911) c5(2912, 2913, 2914, 2915, 2916, 2917) c6( )c7(bL, T, Pt, aw, yY, ew, bB, aj, Kh, bX, aoz, aR, J, ad, eW, Kx, cc, no, aV, lu, aN, Ak, aU, yu, vc, adV, DG, agW, K, KU, an, af, ev, KH, dq, aq, Ku, bQ, cm, kC, dr, aeb, dR, wT, rR, ky, kB, dB, lq, Kv, bE, mx, U, gZ, oh, mf, aan, bC, alq, bye, ao); #c1(FXN) c2(NP_001155178) c3(frataxin, mitochondrial isoform 3 preproprotein) c4(61498) c5(61499, 61500, 61501, 61502, 61503, 61504) c6( )c7(bL, T, Pt, aw, yY, ew, bB, aj, Kh, bX, aoz, aR, J, ad, eW, Kx, cc, no, aV, lu, aN, Ak, aU, yu, vc, adV, DG, agW, K, KU, an, af, ev, KH, dq, aq, Ku, bl, cm, kC, dr, aeb, dR, wT, rR, ky, kB, dB, lq, Kv, bE, mx, U, gZ, oh, mf, aan, bC, alq, bye, ao); #c1 (FXN) c2(NP_852090) c3(frataxin, mitochondrial isoform 2 preproprotein) c4(254806) c5(254807, 254808, 254809, 254810, 254811, 254812) c6( )c7(bL, T, Pt, aw, yY, ew, bB, aj, Kh, bX, aoz, aR, J, ad, eW, Kx, cc, no, aV, lu, aN, Ak, aU, yu, vc, adV, DG, agW, K, KU, an, af, ev, KH, dq, aq, Ku, bl, cm, kC, dr, aeb, dR, wT, rR, ky, kB, dB, lq, Kv, bE, mx, U, gZ, oh, mf, aan, bC, alq, bye, ao); #c1(FXR1) c2(NP_001013456) c3(fragile X mental retardation syndrome-related protein 1 isoform b) c4(25610) c5(25611, 25612, 25613, 25614, 25615, 25616) c6( )c7(T, ff, h, Wb, rx, Ku, nA, en, g, ae, ad); #c1(FXRI) c2(NP_001013457) c3(fragile X mental retardation syndrome-related protein 1 isoform c) c4(25617) c5(25618, 25619, 25620, 25621, 25622) c6( )c7(T, ff, h, Wb, rx, Ku, nA, en, g, ae, ad); #c1(FXRI) c2(NP_005078) c3(fragile X mental retardation syndrome-related protein 1 isoform a) c4(152691, 152692) c5(152693, 152694, 152695, 152696, 152697, 152698, 152699, 152700, 152701, 152702, 152703, 152704, 152705) c6( )c7(T, ff, h, Wb, rx, Ku, nA, en, g, ae, ad); #c1(FXR2) c2(NP_004851) c3(fragile X mental retardation syndrome-related protein 2) c4(150310) c5(150311, 150312, 150313, 150314, 150315, 150316) c6( )c7(h, Ku); #c1(FXYD1) c2(NP_001265647) c3(phospholemman precursor) c4(99098) c5(99099, 99100, 99101, 99102, 99103, 99104, 99105, 99106, 99107, 99108) c6( )c7(Qi, q, j, r); #c1(FXYD2) c2(NP_001671) c3(sodium/potassium-transporting ATPase subunit gamma isoform 1) c4(119895) c5(119896, 119897, 119898, 119899, 119900, 119901) c6( )c7(byf, aj, J, cz); #c1(FXYD2) c2(NP_067614) c3(sodium/potassium-transporting ATPase subunit gamma isoform 2) c4(209066) c5(209067, 209068, 209069, 209070, 209071, 209072, 209073, 209074, 209075, 209076) c6( )c7(byf, aj, J, cz); #c1(FXYD3) c2(NP_001129479) c3(FXYD domain-containing ion transport regulator 3 isoform 3) c4(51561, 51562) c5(51563, 51564, 51565, 51566, 51567, 51568, 51569, 51570, 51571, 51572, 51573, 51574) c6( )c7(ae, T, aC, aE, r, aT, kp, ci, bz, q, ad); #c1(FXYD3) c2(NP_001129480) c3(FXYD domain-containing ion transport regulator 3 isoform 4 precursor) c4( )c5(51575, 51576, 51577, 51578, 51579) c6( )c7(ae, T, aC, aE, r, aT, kp, ci, bz, q, ad); #c1(FXYD3) c2(NP_001129482) c3(FXYD domain-containing ion transport regulator 3 isoform 5 precursor) c4(51580) c5(51581, 51582, 51583, 51584, 51585, 51586) c6( )c7(ae, T, aC, aE, r, aT, kp, ci, bz, q, ad); #c1(FXYD3) c2(NP_001129483) c3(FXYD domain-containing ion transport regulator 3 isoform 1 precursor) c4( )c5(51587, 51588, 51589, 51590, 51591, 51592, 51593, 51594) c6( )c7(ae, T, aC, aE, r, aT, kp, ci, bz, q, ad); #c1(FXYD3) c2(XP_005259053) c3 (FXYD domain-containing ion transport regulator 3 isoform X1) c4( )c5(275936, 275937, 275938, 275939, 275940) c6( )c7(ae, T, aC, aE, r, aT, kp, ci, bz, q, ad); #c1(FXYD3) c2(XP_011525334) c3(FXYD domain-containing ion transport regulator 3 isoform X2) c4(319801, 319802) c5(319803, 319804) c6( )c7(ae, T, aC, aE, r, aT, kp, ci, bz, q, ad); #c1(FXYD5) c2(NP_659003) c3(FXYD domain-containing ion transport regulator 5 precursor) c4(239682, 239683, 239684) c5(239685, 239686, 239687, 239688, 239689, 239690, 239691, 239692, 239693, 239694, 239695, 239696, 239697, 239698, 239699, 239700, 239701, 239702, 239703, 239704, 239705, 239706, 239707, 239708) c6( )c7(T, aC, wu, Or, aA, ci, iJ, byh, aE, byg, aT, K, wm, hH, aW, Y, ff, aq, en, bu, iR, eD, RW, pg, adR, bt, B, ao); #c1(FXYD5) c2(XP_006723309) c3(FXYD domain-containing ion transport regulator 5 isoform X1) c4(295116) c5(295117, 295118, 295119, 295120, 295121, 295122) c6( )c7(T, aC, wu, Or, aA, ci, iJ, byh, aE, byg, aT, K, wm, hH, aW, Y, ff, aq, en, bu, iR, eD, RW, pg, adR, bt, B, ao); #c1(FXYD5) c2(XP_006723310) c3(FXYD domain-containing ion transport regulator 5 isoform X2) c4( )c5(295123, 295124, 295125, 295126, 295127, 295128) c6( )c7(T, aC, wu, Or, aA, ci, iJ, byh, aE, byg, aT, K, wm, hH, aW, Y, ff, aq, en, bu, iR, eD, RW, pg, adR, bt, B, ao); #c1(FXYD6) c2(NP_001158303) c3(FXYD domain-containing ion transport regulator 6 precursor) c4(63572) c5(63573, 63574, 63575, 63576, 63577, 63578, 63579, 63580, 63581, 63582, 63583, 63584, 63585, 63586, 63587, 63588, 63589, 63590, 63591, 63592, 63593, 63594, 63595, 63596, 63597, 63598) c6( )c7(bt); #c1(FYB) c2(NP_001230022) c3(FYN-binding protein isoform 3) c4(85797, 85798, 85799, 85800) c5(85801, 85802, 85803, 85804, 85805, 85806, 85807, 85808, 85809, 85810, 85811, 85812, 85813, 85814, 85815, 85816, 85817, 85818, 85819, 85820, 85821, 85822, 85823, 85824, 85825, 85826, 85827, 85828, 85829, 85830, 85831) c6( )c7(anX, cy, fJ, dV, hX); #c1(FYB) c2(NP_955367) c3(FYN-binding protein isoform 2) c4( )c5(261582, 261583, 261584, 261585, 261586, 261587, 261588, 261589) c6( )c7(anX, cy, fJ, dV, hX); #c1(FYB) c2(XP_011512313) c3(FYN-binding protein isoform X3) c4(302859) c5(302860, 302861) c6( )c7(anX, cy, fJ, dV, hX); #c1(FYB) c2(XP_011512314) c3(FYN-binding protein isoform X4) c4(302862, 302863, 302864) c5( ) c6( )c7(anX, cy, fJ, dV, hX); #c1(FYB) c2(XP_011512315) c3(FYN-binding protein isoform X5) c4( )c5(302865, 302866, 302867, 302868, 302869, 302870, 302871, 302872, 302873, 302874, 302875, 302876, 302877, 302878) c6( )c7(anX, cy, fJ, dV, hX); #c1(FYC01) c2(NP_078789) c3(FYVE and coiled-coil domain-containing protein 1) c4(215455, 215456, 215457, 215458) c5(215459, 215460, 215461, 215462, 215463, 215464, 215465, 215466, 215467, 215468, 215469, 215470, 215471, 215472, 215473, 215474, 215475, 215476) c6( )c7(hH, byi); #c1(FYC01) c2(XP_011532414) c3(FYVE and coiled-coil domain-containing protein 1 isoform X2) c4( )c5(328602, 328603, 328604, 328605) c6( )c7(hH, byi); #c1(FYN) c2(NP_002028) c3(tyrosine-protein kinase Fyn isoform a) c4(123449) c5(123450, 123451, 123452, 123453, 123454, 123455, 123456, 123457, 123458, 123459) c6( )c7(bM, T, aAx, aA, GC, Fb, j, C, il, eA, ad, x, zw, ib, aT, h, bK, K, an, ae, af, bA, ak, yo, Pw, d, A, s, aW, ff, eZ, zC, en, g, cm, hq, ky, Gs, U, k, aen); #c1(FYN) c2(XP_005266947) c3(tyrosine-protein kinase Fyn isoform X2) c4(280420, 280421, 280422, 280423, 280424, 280425, 280426) c5(280427, 280428, 280429, 280430, 280431, 280432, 280433, 280434, 280435, 280436, 280437, 280438, 280439, 280440, 280441, 280442, 280443, 280444, 280445, 280446, 280447, 280448, 280449, 280450, 280451, 280452) c6( )c7(bM, T, aAx, aA, GC, Fb, j, C, il, eA, ad, x, zw, ib, aT, h, bK, K, an, ae, af, bA, ak, yo, Pw, d, A, s, aW, ff, eZ, zC, en, g, cm, hq, ky, Gs, U, k, aen); #c1(FYN) c2(XP_005266949) c3(tyrosine-protein kinase Fyn isoform X1) c4(280453, 280454, 280455, 280456, 280457, 280458) c5(280459, 280460, 280461, 280462, 280463, 280464, 280465, 280466, 280467, 280468, 280469, 280470, 280471, 280472, 280473, 280474, 280475, 280476, 280477, 280478, 280479, 280480, 280481, 280482, 280483, 280484, 280485, 280486, 280487, 280488, 280489, 280490, 280491, 280492, 280493, 280494, 280495, 280496, 280497, 280498, 280499, 280500, 280501, 280502, 280503, 280504, 280505, 280506, 280507, 280508, 280509, 280510, 280511, 280512, 280513, 280514, 280515, 280516, 280517, 280518, 280519, 280520, 280521, 280522, 280523, 280524, 280525, 280526, 280527, 280528, 280529, 280530, 280531, 280532, 280533, 280534, 280535, 280536, 280537, 280538, 280539, 280540, 280541, 280542, 280543, 280544, 280545, 280546) c6( )c7(bM, T, aAx, aA, GC, Fb, j, C, il, eA, ad, x, zw, ib, aT, h, bK, K, an, ae, af, bA, ak, yo, Pw, d, A, s, aW, ff, eZ, zC, en, g, cm, hq, ky, Gs, U, k, aen); #c1(FYN) c2(XP_011533970) c3(tyrosine-protein kinase Fyn isoform X3) c4(330173, 330174, 330175, 330176) c5( )c6( )c7(bM, T, aAx, aA, GC, Fb, j, C, il, eA, ad, x, zw, ib, aT, h, bK, K, an, ae, af, bA, ak, yo, Pw, d, A, s, aW, ff, eZ, zC, en, g, cm, hq, ky, Gs, U, k, aen); #c1 (FZD1) c2(NP_003496) c3(frizzled-1) c4(138769) c5(138770, 138771, 138772, 138773, 138774, 138775, 138776, 138777) c6( )c7(T, ht, bB, Hn, bS, ct, aT, hu, jf, byj, aW, eH, eF, cm, Z, t, HI, qJ, LB, alC, kP, nB, bE, iS, byk); #c1(FZD10) c2(NP_009128) c3(frizzled-10 precursor) c4(170952) c5(170953, 170954, 170955, 170956, 170957, 170958, 170959, 170960, 170961, 170962) c6( )c7(T, bE, if, bhU, r, bB, qN, he, iJ); #c1(FZD2) c2(NP_001457) c3(frizzled-2 precursor) c4(117963, 117964) c5(117965, 117966, 117967, 117968, 117969, 117970, 117971, 117972, 117973, 117974, 117975, 117976) c6( )c7(LB, bF, T, jf, vA, ht, aw, aT, bK, ci, hu, er, X, op); #c1(FZD3) c2(NP_665873) c3(frizzled-3 precursor) c4(241885, 241886) c5(241887, 241888, 241889, 241890, 241891, 241892, 241893, 241894, 241895, 241896) c6( )c7(bF, ul, alC, BN, s, cA, do, cj, gl, hy, Z, ne); #c1(FZD3) c2(XP_011542950) c3(frizzled-3 isoform X3) c4( ) c5(341657, 341658, 341659, 341660, 341661, 341662) c6( )c7(bF, ul, alC, BN, s, cA, d0, cj, gl, hy, Z, ne); #c1(FZD4) c2(NP_036325) c3(frizzled-4 precursor) c4(173025) c5(173026, 173027, 173028, 173029, 173030, 173031, 173032) c6( )c7(bL, bM, T, n, hy, Ja, v, zU, eA, ad, Na, aE, pX, BJ, h, dL, he, Fg, ae, eL, d, BN, byj, lR, nO, Y, ff, Az, vt, byl, en, vk, W, nb, ajw, Hs, dX, ol, bt, cE, ne); #c1(FZD5) c2(NP_003459) c3(frizzled-5 precursor) c4(138477) c5(138478, 138479, 138480, 138481, 138482, 138483) c6( )c7(T, ht, aA, bB, Hn, bS, ct, ci, hu, H, byj, eH, cr, aF, eF, Z, HI, cm, es, alC, kP, bE, Be, iS, Bd, byk, L); #c1(FZD6) c2(NP_001158088) c3(frizzled-6 isoform b) c4(63101, 63102) c5(63103, 63104, 63105, 63106, 63107, 63108, 63109, 63110, 63/11, 63112, 63113) c6( )c7(nM, s, bym, byn, cj, hR, bS, ct, IQ, Z, cm, byk); #c1(FZD6) c2(NP_003497) c3(frizzled-6 isoform a precursor) c4(138778) c5(138779, 138780, 138781, 138782, 138783, 138784, 138785, 138786, 138787, 138788, 138789) c6( )c7(nM, s, bym, byn, cj, hR, bS, ct, lQ, Z, cm, byk); #c1(FZD7) c2(NP_003498) c3(frizzled-7 precursor) c4(138790) c5(138791, 138792, 138793, 138794, 138795, 138796) c6( )c7(bF, T, B, kP, bE, X, ht, r, aA, bB, at, dL, aF, ci, jE, hu, q, er, hX); #c1(FZD8) c2(NP_114072) c3(frizzled-8 precursor) c4(222128) c5(222129, 222130, 222131, 222132, 222133, 222134) c6( )c7(el, dL, kP); #c1(FZD9) c2(NP_003499) c3 (frizzled-9 precursor) c4(138797) c5(138798, 138799, 138800, 138801, 138802, 138803, 138804) c6( )c7(bF, av, ul, alC, s, Pt, eM, atB, dO, r, cj, aA, bw, aF, he, bt, q, bN); #c1(FZR1) c2(NP_001129669) c3(fizzy-related protein homolog isoform 3) c4(52169) c5(52170, 52171, 52172, 52173, 52174, 52175) c6( )c7(bM, T, pf, if, fd, bB, ci, ajv, gs, eL, alo, YL, yo, aW, az, ir, g, as, eF, eM, m, alm, in, Uc, oO, sw, n, fr, qN, pM, Bl, jz, dL, hB, kp, mo, fs, hL, it, B, fM, aA, pd, q, er, eA, XX, hW, aT, ae, jf, avg, s, iU, Y, r, en, avb, cl, t, ik, nM, hS, Mb, id, aD, iW, ad, iC, av, hd, py, fb, BO, Ub, qt, ff, eH, aF, iV, bz, bF, es, apM, bE, ra, iu); #c1(FZR1) c2(NP_001129670) c3(fizzy-related protein homolog isoform 1) c4(52176) c5(52177, 52178, 52179, 52180, 52181, 52182) c6( )c7(bM, T, pf, if, fd, bB, ci, ajv, gs, eL, alo, YL, yo, aW, az, ir, g, as, eF, eM, m, alm, in, Uc, QO, sw, n, fr, qN, pM, Bl, jz, dL, hB, kp, mo, fs, hL, it, B, fM, aA, pd, q, er, eA, XX, hW, aT, ae, jf, avg, s, il, Y, r, en, avb, cl, t, iK, nM, hS, Mb, id, aD, iW, ad, iC, av, hd, py, fb, BO, Ub, qt, ff, eH, aF, iV, bz, bF, es, apM, bE, ra, iu); #c1(FZR1) c2(NP_057347) c3(fizzy-related protein homolog isoform 2) c4(190246) c5(190247, 190248, 190249, 190250, 190251, 190252) c6( )c7(bM, T, pf, if, fd, bB, ci, ajv, gs, eL, alo, YL, yo, aW, az, ir, g, as, eF, eM, m, alm, in, Uc, oO, sw, n, fr, qN, pM, Bl, jz, dL, hB, kp, mo, fs, hL, it, B, fM, aA, pd, q, er, eA, XX, hW, aT, ae, jf, avg, s, iU, Y, r, en, avb, cl, t, iK, nM, hS, Mb, id, aD, iW, ad, iC, av, hd, py, fb, BO, Ub, qt, ff, eH, aF, iV, bz, bF, es, apM, bE, ra, iu); #c1(GOS2) c2(NP_056529) c3(G0/Gl switch protein 2) c4(187957) c5(187958, 187959, 187960, 187961, 187962, 187963) c6( )c7(bL, ae, T, y, zQ, dq, v, ci, jj, zP, bz, ad); #c1(G2E3) c2(NP_060239) c3(G2/M phase-specific E3 ubiquitin-protein ligase) c4(194911, 194912) c5(194913, 194914, 194915, 194916, 194917, 194918, 194919, 194920, 194921, 194922, 194923, 194924) c6( )c7(bM); #c1(G2E3) c2(XP_011535228) c3(G2/M phase-specific E3 ubiquitin-protein ligase isoform X2) c4(332045, 332046, 332047, 332048) c5( )c6( )c7(bM); #c1(G2E3) c2(XP_011535230) c3(G2/M phase-specific E3 ubiquitin-protein ligase isoform X5) c4( )c5(332049, 332050, 332051, 332052, 332053, 332054, 332055, 332056, 332057, 332058, 332059, 332060, 332061, 332062, 332063, 332064, 332065, 332066, 332067, 332068, 332069, 332070, 332071, 332072) c6( )c7(bM); #c1(G3BPI) c2(XP_006714813) c3(ras GTPase-activating protein-binding protein 1 isoform X1) c4(288177, 288178, 288179, 288180, 288181, 288182, 288183) c5(288184, 288185, 288186, 288187, 288188, 288189, 288190, 288191, 288192, 288193, 288194, 288195, 288196, 288197, 288198, 288199, 288200, 288201, 288202, 288203, 288204, 288205, 288206, 288207, 288208, 288209, 288210, 288211, 288212, 288213, 288214, 288215, 288216, 288217, 288218, 288219, 288220, 288221, 288222, 288223, 288224, 288225, 288226, 288227, 288228, 288229, 288230, 288231, 288232, 288233) c6( )c7(Dw, T, aE, aW, lH, jx, ff, aT, By, ey, K, en, y, bz); #c1(G6PC) c2(NP_000142) c3(glucose-6-phosphatase isoform 1) c4(2952) c5(2953, 2954, 2955, 2956, 2957, 2958, 2959, 2960, 2961, 2962, 2963, 2964, 2965, 2966, 2967, 2968, 2969, 2970, 2971, 2972, 2973, 2974, 2975, 2976, 2977, 2978, 2979, 2980, 2981, 2982, 2983, 2984, 2985, 2986, 2987, 2988, 2989, 2990, 2991, 2992, 2993, 2994, 2995, 2996, 2997, 2998, 2999, 3000, 3001, 3002, 3003, 3004, 3005, 3006, 3007, 3008, 3009, 3010, 3011, 3012, 3013, 3014, 3015, 3016, 3017, 3018, 3019, 3020, 3021, 3022, 3023, 3024, 3025, 3026, 3027, 3028, 3029, 3030, 3031, 3032, 3033, 3034, 3035, 3036, 3037, 3038, 3039, 3040, 3041, 3042, 3043, 3044, 3045, 3046, 3047, 3048, 3049, 3050, 3051, 3052, 3053, 3054, 3055, 3056, 3057, 3058, 3059, 3060, 3061, 3062, 3063, 3064, 3065, 3066, 3067, 3068, 3069, 3070, 3071, 3072, 3073, 3074, 3075, 3076, 3077, 3078, 3079, 3080, 3081, 3082, 3083, 3084, 3085, 3086, 3087, 3088, 3089, 3090, 3091, 3092, 3093, 3094, 3095) c6( )c7(bL, gV, T, ZF, afK, dG, aj, ci, fr, vY, aY, eA, J, byp, nH, Yn, mj, byq, ju, io, jy, dz, bq, hB, ex, ak, kl, d, A, s, hG, Bw, fc, dq, aq, EZ, dr, byo, SE, dB, acY, bo, mf, dy, cP, B); #c1(G6PC2) c2(NP_001075155) c3 (glucose-6-phosphatase 2 isoform 2) c4(37260) c5(37261, 37262, 37263, 37264, 37265, 37266) c6( )c7(bL, dD, A, yG, Bw, mg, GP, jy, aj, mf, dG, v, ei, eq, J); #c1(G6PC2) c2(NP_066999) c3(glucose-6-phosphatase 2 isoform 1) c4(208210) c5(208211, 208212, 208213, 208214, 208215, 208216) c6( )c7(bL, dD, A, yG, Bw, mg, GP, jy, aj, mf, dG, v, ei, eq, J); #c1(GGPC3) c2(NP_612396) c3(glucose-6-phosphatase 3) c4(236370) c5(236371, 236372, 236373, 236374, 236375, 236376) c6( )c7(gV, byt, Gc, hG, XE, ex, CM, bys, ul, fe, byr, afK, aoF, K, C, AE, bD, bw, bt, br); #c1(G6PD) c2(NP_000393) c3(glucose-6-phosphate 1-dehydrogenase isoform a) c4(8449) c5(8450, 8451, 8452, 8453, 8454, 8455, 8456, 8457, 8458) c6( )c7(T, qo, Fj, fR, e, fd, x, dV, K, hu, ex, qD, byv, aW, hC, uY, Ku, hJ, byH, bw, eY, xK, aDs, cj, iS, k, pw, Cl, hh, eg, byy, gp, aWG, ht, aFc, mN, xJ, aj, nA, cd, br, aCi, b, cc, byF, KN, h, lG, byw, A, aq, pm, afj, byl, byz, yN, byG, E, ER, B, bL, agx, rf, v, j, qf, C, byu, J, oT, boY, MR, aCX, nX, byE, aT, dz, bK, bq, yj, avV, iU, oV, Y, byA, lo, qs, zz, hY, ck, byB, BU, byx, ac, VJ, hO, mf, oX, bt, byD, Ke, qb, iW, KW, KO, bmJ, agz, hX, l, qh, oj, oW, iY, hP, NM, aHp, byC, JU, Fm, xs, nf, dq, S, BG, avG, aot, Pd, iR, dr, ek, adA, mO, adc, Ew, qe, pc, eB, Km, bC, ei, i, lC, X); #c1(G6PD) c2(NP_001035810) c3(glucose-6-phosphate 1-dehydrogenase isoform b) c4(33142, 33143, 33144) c5(33145, 33146, 33147, 33148, 33149, 33150, 33151, 33152, 33153, 33154, 33155, 33156, 33157) c6( )c7(T, qo, Fj, fR, e, fd, x, dV, K, hu, ex, qD, byv, aW, hC, uY, Ku, hJ, byH, bw, eY, xK, aDs, cj, iS, k, pw, Cl, hh, eg, byy, gp, aWG, ht, aFc, mN, xJ, aj, nA, cd, br, aCi, b, cc, byF, KN, h, lG, byw, A, aq, pm, afj, byl, byz, yN, byG, E, ER, B, bL, agx, rf, v, j, qf, C, byu, J, oT, boY, MR, aCX, nX, byE, aT, dz, bK, bq, yj, avV, iU, oV, Y, byA, lo, qs, zz, hY, ck, byB, BU, byx, ac, VJ, hO, mf, oX, bt, byD, Ke, qb, iW, KW, KO, bmJ, agz, hX, l, qh, oj, oW, iY, hP, NM, aHp, byC, JU, Fm, xs, nf, dq, S, BG, avG, aot, Pd, iR, dr, ek, adA, ml, adc, Ew, qe, pc, eB, Km, bC, ei, i, lC, X); #c1(G6PD) c2(XP_011529434) c3(glucose-6-phosphate 1-dehydrogenase isoform X3) c4( )c5(324869, 324870, 324871, 324872, 324873, 324874) c6( )c7(T, qo, Fj, fR, e, fd, x, dV, K, hu, ex, qD, byv, aW, hC, uY, Ku, hJ, byH, bw, eY, xK, aDs, cj, iS, k, pw, Cl, hh, eg, byy, gp, aWG, ht, aFc, mN, xJ, aj, nA, cd, br, aCi, b, cc, byF, KN, h, lG, byw, A, aq, pm, aFj, byl, byz, yN, byG, E, ER, B, bL, agx, rf, v, j, qf, C, byu, J, oT, boY, MR, aCX, nX, byE, aT, dz, bK, bq, yj, avV, iU, oV, Y, byA, lo, qs, zz, hY, ck, byB, BU, byx, ac, VJ, hO, mf, oX, bt, byD, Ke, qb, iW, KW, KO, bmJ, agz, hX, l, qh, oj, oW, iY, hP, NM, aHp, byC, JU, Fm, xs, nf, dq, S, BG, avG, aot, Pd, iR, dr, ek, adA, mO, adc, Ew, qe, pc, eB, Km, bC, ei, i, lC, X); #c1(GAA) c2(XP_005257251) c3(lysosomal alpha-glucosidase isoform XI) c4(274813) c5(274814, 274815, 274816, 274817, 274818, 274819, 274820, 274821, 274822, 274823, 274824, 274825, 274826, 274827, 274828, 274829, 274830, 274831, 274832, 274833, 274834, 274835, 274836, 274837, 274838, 274839, 274840, 274841, 274842, 274843, 274844, 274845, 274846, 274847, 274848, 274849, 274850, 274851, 274852, 274853, 274854, 274855, 274856, 274857, 274858, 274859, 274860, 274861, 274862, 274863, 274864, 274865, 274866, 274867, 274868, 274869, 274870, 274871, 274872, 274873, 274874, 274875, 274876, 274877, 274878, 274879, 274880, 274881, 274882, 274883, 274884, 274885, 274886, 274887, 274888, 274889, 274890, 274891, 274892, 274893, 274894, 274895, 274896, 274897, 274898, 274899, 274900, 274901, 274902, 274903, 274904, 274905, 274906, 274907, 274908, 274909, 274910, 274911, 274912, 274913, 274914, 274915, 274916, 274917, 274918, 274919, 274920, 274921, 274922, 274923, 274924, 274925, 274926, 274927, 274928, 274929, 274930, 274931, 274932, 274933, 274934, 274935, 274936, 274937, 274938, 274939, 274940, 274941, 274942, 274943, 274944, 274945, 274946, 274947, 274948, 274949, 274950, 274951, 274952, 274953, 274954, 274955, 274956, 274957, 274958, 274959, 274960, 274961, 274962, 274963, 274964, 274965, 274966, 274967, 274968, 274969, 274970, 274971, 274972, 274973, 274974, 274975, 274976, 274977, 274978, 274979, 274980, 274981, 274982, 274983, 274984, 274985, 274986, 274987, 274988, 274989, 274990, 274991, 274992, 274993, 274994, 274995, 274996, 274997, 274998, 274999, 275000, 275001) c6(275002, 275003, 275004, 275005, 275006, 275007) c7(bL, gU, ads, Pt, dk, aA, bjW, aj, ci, vY, J, cc, db, oj, aT, vH, nm, vc, bj, K, adV, agW, af, aa, byJ, bf, aW, Y, oV, Bw, azP, cr, UJ, ax, EH, aF, Ku, dR, wT, BU, W, nb, kB, mx, U, gZ, pg, wR, Lq, mf, bk, be, byK, sT, y, Ro); #c1(GABI) c2(NP_002030) c3(GRB2-associated-binding protein 1 isoform b) c4(123460, 123461) c5(123462, 123463, 123464, 123465, 123466, 123467, 123468, 123469, 123470, 123471, 123472, 123473) c6( )c7(T, aZ, fd, yO, ci, iJ, ad, biS, cF, aT, ae, kl, d, aW, Y, kt, ik, bz, kT, fT, cj, B); #c1(GABI) c2(NP_997006) c3(GRB2-associated-binding protein 1 isoform a) c4(264949) c5(264950, 264951, 264952, 264953, 264954, 264955) c6( )c7(T, aZ, fd, yO, ci, iJ, ad, biS, cF, aT, ae, kl, d, aW, Y, kt, ik, bz, kT, fT, cj, B); #c1(GAB2) c2(NP_036428) c3(GRB2-associated-binding protein 2 isoform b) c4(173795, 173796, (73797) c5(173798, 173799, 173800, 173801, 173802, 173803, 173804, 173805, 173806, 173807) c6( )c7(T, dA, zs, fd, v, ci, eA, yt, aT, h, K, af, iU, aW, Y, gK, t, bw, bz, cT, ig, Om, at, m, y, bt, L); #c1(GAB2) c2(NP_536739) c3(GRB2-associated-binding protein 2 isoform a) c4(231317) c5(231318, 231319, 231320, 231321, 231322, 231323, 231324, 231325, 231326, 231327, 231328, 231329, 231330, 231331) c6( ) c7(T, dA, zs, fd, v, ci, eA, yt, aT, h, K, af, iU, aW, Y, gK, t, bw, bz, cT, ig, Om, at, m, y, bt, L); #c1(GAB3) c2(NP_001075042) c3 (GRB2-associated-binding protein 3 isoform 1) c4( )c5(37232, 37233, 37234, 37235, 37236) c6( )c7(A, fR); #c1(GAB3) c2(NP_542179) c3(GRB2-associated-binding protein 3 isoform 2) c4(232241) c5(232242, 232243, 232244, 232245, 232246, 232247) c6( )c7(A, fR); #c1(GAB3) c2(XP_011529405) c3(GRB2-associated-binding protein 3 isoform X2) c4(324785) c5(324786) c6( )c7(A, fR); #c1(GABARAP) c2(NP_009209) c3(gamma-aminobutyric acid receptor-associated protein) c4(171525) c5(171526, 171527, 171528, 171529, 171530, 171531) c6( )c7(ib, XY, ha, in, aT); #c1 (GABARAPL1) c2(NP_113600) c3(gamma-aminobutyric acid receptor-associated protein-like 1) c4(221633, 221634) c5(221635, 221636, 221637, 221638, 221639, 221640, 221641, 221642, 221643, 221644, 221645, 221646, 221647, 221648, 221649, 221650, 221651, 221652, 221653, 221654, 221655, 221656) c6( )c7(T, aE, aW, U, aT, n, im, t); #c1(GABARAPL1) c2(XP_005253401) c3(gamma-aminobutyric acid receptor-associated protein-like 1 isoform X1) c4(272466, 272467) c5(272468, 272469, 272470, 272471, 272472, 272473, 272474, 272475, 272476, 272477) c6( )c7(T, aE, aW, U, aT, n, im, t); #c1(GABARAPL2) c2(NP_009216) c3(gamma-aminobutyric acid receptor-associated protein-like 2) c4(171555) c5(171556, 171557, 171558, 171559, 171560, 171561, 171562, 171563) c6( ) c7(aE); #c1(GABBR1) c2(NP_001461) c3(gamma-aminobutyric acid type B receptor subunit 1 isoform a precursor) c4(117977, 117978, 117979) c5( )c6( )c7(T, aC, ha, bO, cz, iD, ci, il, V, cF, ft, Gt, aE, wM, aql, bq, rE, he, af, Gv, eZ, cu, bN, Cq, aap, GD, HN, yA, gD, qc); #c1(GABBR1) c2(NP_068703) c3(gamma-aminobutyric acid type B receptor subunit 1 isoform b precursor) c4(209967) c5(209968, 209969, 209970, 209971, 209972, 209973, 209974, 209975, 209976, 209977, 209978, 209979, 209980, 209981, 209982, 209983, 209984, 209985, 209986, 209987, 209988, 209989, 209990, 209991, 209992, 209993, 209994, 209995, 209996, 209997, 209998, 209999, 210000, 210001, 210002, 210003, 210004, 210005, 210006, 210007, 210008, 210009, 210010, 210011, 210012, 210013, 210014, 210015, 210016, 210017, 210018, 210019, 210020, 210021, 210022, 210023, 210024, 210025, 210026, 210027, 210028, 210029, 210030, 210031, 210032, 210033, 210034, 210035, 210036, 210037, 210038, 210039, 210040, 210041, 210042, 210043, 210044, 210045, 210046, 210047, 210048) c6(210049, 210050, 210051, 210052, 210053, 210054, 210055, 210056, 210057, 210058, 210059, 210060, 210061, 210062, 210063, 210064, 210065, 210066, 210067, 210068, 210069, 210070, 210071, 210072) c7(T, aC, ha, bO, cz, iD, ci, il, V, cF, ft, Gt, aE, wM, aql, bq, rE, he, af, Gv, eZ, cu, bN, Cq, aap, GD, HN, yA, gD, qc); #c1(GABBR1) c2(NP_068704) c3(gamma-aminobutyric acid type B receptor subunit 1 isoform c precursor) c4(210073, 210074) c5( )c6( )c7(T, aC, ha, bO, cz, iD, ci, il, V, cF, ft, Gt, aE, wM, aql, bq, rE, he, af, Gv, eZ, cu, bN, Cq, aap, GD, HN, yA, gD, qc); #c1(GABBR1) c2(XP_005249039) c3(gamma-aminobutyric acid type B receptor subunit 1 isoform X1) c4( )c5(268881, 268882, 268883, 268884, 268885, 268886, 268887, 268888, 268889, 268890, 268891, 268892, 268893, 268894, 268895, 268896, 268897, 268898, 268899, 268900, 268901, 268902, 268903, 268904, 268905, 268906, 268907, 268908, 268909, 268910, 268911, 268912, 268913, 268914, 268915, 268916, 268917, 268918, 268919, 268920, 268921, 268922) c6(268923, 268924, 268925, 268926, 268927, 268928, 268929, 268930, 268931, 268932, 268933, 268934, 268935, 268936) c7(T, aC, ha, bO, cz, iD, ci, il, V, cF, ft, Gt, aE, wM, aql, bq, rE, he, af, Gv, eZ, cu, bN, Cq, aap, GD, HN, yA, gD, qc); #c1(GABBR1) c2(XP_005275145) c3(gamma-aminobutyric acid type B receptor subunit 1 isoform X1) c4(285544, 285545) c5(285546, 285547, 285548, 285549, 285550, 285551) c6( )c7(T, aC, ha, bO, cz, iD, ci, il, V, cF, ft, Gt, aE, wM, aql, bq, rE, he, af, Gv, eZ, cu, bN, Cq, aap, GD, HN, yA, gD, qc); #c1(GABBR1) c2(XP_006715110) c3(gamma-aminobutyric acid type B receptor subunit 1 isoform X2) c4( ) c5(288650, 288651, 288652, 288653, 288654, 288655, 288656, 288657, 288658, 288659, 288660, 288661, 288662, 288663, 288664, 288665, 288666, 288667, 288668, 288669, 288670, 288671, 288672, 288673, 288674, 288675, 288676, 288677, 288678, 288679, 288680, 288681, 288682, 288683, 288684, 288685, 288686, 288687, 288688, 288689, 288690) c6(288691, 288692, 288693, 288694, 288695, 288696, 288697, 288698, 288699, 288700, 288701, 288702, 288703, 288704) c7(T, aC, ha, bO, cz, iD, ci, il, V, cF, ft, Gt, aE, wM, aql, bq, rE, he, af, Gv, eZ, cu, bN, Cq, aap, GD, HN, yA, gD, qc); #c1(GABBR1) c2(XP_006725870) c3(gamma-aminobutyric acid type B receptor subunit 1 isoform X2) c4( )c5(297014, 297015, 297016, 297017, 297018, 297019) c6( )c7(T, aC, ha, bO, cz, iD, ci, il, V, cF, ft, Gt, aE, wM, aql, bq, rE, he, af, Gv, eZ, cu, bN, Cq, aap, GD, HN, yA, gD, qc); #c1(GABBR1) c2(XP_011512758) c3(gamma-aminobutyric acid type B receptor subunit 1 isoform X6) c4( )c5(303789, 303790, 303791, 303792, 303793, 303794, 303795, 303796, 303797, 303798, 303799, 303800, 303801, 303802, 303803, 303804, 303805, 303806, 303807, 303808, 303809, 303810, 303811, 303812, 303813, 303814, 303815, 303816, 303817, 303818, 303819, 303820, 303821, 303822, 303823, 303824, 303825, 303826, 303827, 303828, 303829, 303830, 303831) c6(303832, 303833, 303834, 303835, 303836, 303837, 303838, 303839, 303840, 303841, 303842, 303843, 303844, 303845) c7(T, aC, ha, bO, cz, iD, ci, il, V, cF, ft, Gt, aE, wM, aql, bq, rE, he, af, Gv, eZ, cu, bN, Cq, aap, GD, HN, yA, gD, qc); #c1(GABBR1) c2(XP_011546151) c3(gamma-aminobutyric acid type B receptor subunit 1 isoform X6) c4( )c5(344860, 344861, 344862, 344863, 344864, 344865) c6( )c7(T, aC, ha, bO, cz, iD, ci, il, V, cF, ft, Gt, aE, wM, aql, bq, rE, he, af, Gv, eZ, cu, bN, Cq, aap, GD, HN, yA, gD, qc); #c1(GABBR2) c2(NP_005449) c3(gamma-aminobutyric acid type B receptor subunit 2 precursor) c4(156284) c5(156285, 156286, 156287, 156288, 156289, 156290) c6( )c7(Na, bM, xU, aqh, vj, XY, bO, Nc, bK, an, il, bN); #c1 (GABPA) c2(NP_002031) c3(GA-binding protein alpha chain) c4(123474) c5(123475, 123476, 123477, 123478, 123479, 123480, 123481, 123482, 123483, 123484, 123485) c6( )c7(oO, bM, T, aC, ea, if, e, al, bB, qj, aY, fw, aR, bv, aE, dV, K, aDZ, eL, bc, aa, LC, aW, fc, hl, nY, Qq, g, cm, eM, nB, U, cj, NI, vy, tG, Hb, m, bi, iu, dD, JY, aWG, n, aj, asF, qN, PJ, rP, Hx, ft, cc, bud, cU, h, dl, hB, db, aq, Ye, W, Fi, hL, at, fL, cB, B, bL, eR, yk, hx, fF, cR, v, j, C, V, q, J, er, eA, Wu, aT, cy, jj, ae, af, Lu, s, oV, Y, r, lo, en, cl, t, ck, sk, rR, nM, c, kB, dh, dy, fD, bt, ao, qb, ha, eq, ad, hX, fU, av, hd, ib, byl, oz, oW, io, ji, lQ, d, ff, aF, byM, iR, KM, bz, ek, xZ, bE, aS, cP, tP, kS, X, pC); #c1(GABPB1) c2(NP_002032) c3(GA-binding protein subunit beta-1 isoform gamma 1) c4(123486) c5(123487, 123488, 123489, 123490, 123491, 123492) c6( )c7(Fi, fq, ey, eu, JY); #c1 (GABPBI) c2(NP_057738) c3(GA-binding protein subunit beta-1 isoform beta 2) c4(192593) c5(192594, 192595, 192596, 192597, 192598, 192599) c6( )c7(Fi, fq, ey, eu, JY); #c1(GABPB1) c2(NP_852092) c3(GA-binding protein subunit beta-1 isoform gamma 2) c4(254827) c5(254828, 254829, 254830, 254831, 254832, 254833, 254834, 254835, 254836, 254837, 254838) c6( )c7(Fi, fq, ey, eu, JY); #c1(GABPB1) c2(XP_005254331) c3(GA-binding protein subunit beta-1 isoform X1) c4(273150, 273151) c5(273152, 273153, 273154, 273155, 273156, 273157, 273158) c6( )c7(Fi, fq, ey, eu, JY); #c1(GABPB1) c2(XP_011519729) c3(GA-binding protein subunit beta-1 isoform X4) c4( )c5(314385, 314386, 314387, 314388, 314389, 314390, 314391, 314392, 314393, 314394) c6( )c7(Fi, fq, ey, eu, JY); #c1(GABRA1) c2(NP_001121120) c3(gamma-aminobutyric acid receptor subunit alpha-1 precursor) c4(45213, 45214) c5(45215, 45216, 45217, 45218, 45219, 45220, 45221, 45222, 45223, 45224, 45225, 45226, 45227, 45228, 45229, 45230, 45231, 45232, 45233, 45234, 45235, 45236, 45237, 45238, 45239, 45240, 45241, 45242, 45243, 45244, 45245, 45246, 45247, 45248) c6( )c7(ha, bB, iD, PJ, q, kL, aqt, Gt, byN, aqk, al, bK, byO, d, r, gl, aPf, akq, bN, alT, Cq, ok, cA, bE, Gs, HN, byP, byQ); #c1(GABRA2) c2(NP_000798) c3(gamma-aminobutyric acid receptor subunit alpha-2 isoform a precursor) c4(16151, 16152) c5(16153, 16154, 16155, 16156, 16157, 16158, 16159, 16160, 16161, 16162, 16163, 16164, 16165, 16166, 16167, 16168, 16169, 16/70, 16171, 16172, 16173, 16174, 16175, 16176, 16177, 16178, 16179, 16180, 16181, 16182, 16183, 16184, 16185, 16186, 16187, 16188) c6( )c7(byR, hU, ha, Gd, pk, WV, Si, DK, byS, XY, bK, K, Jc, gC, da, cu, jb, bN, hq, Oh, FT, HP, Gs, Gp, jd, bhP); #c1(GABRA2) c2(NP_001273756) c3(gamma-aminobutyric acid receptor subunit alpha-2 isoform b) c4(105774) c5(105775, 105776, 105777, 105778, 105779, 105780) c6( )c7(byR, hU, ha, Gd, pK, VV, Si, DK, byS, XY, bK, K, Jc, gC, da, cu, jb, bN, hq, Oh, FT, HP, Gs, Gp, jd, bhP); #c1(GABRA2) c2(XP_011511978) c3(gamma-aminobutyric acid receptor subunit alpha-2 isoform X1) c4(302629, 302630, 302631) c5(302632, 302633, 302634, 302635, 302636, 302637) c6( )c7(byR, hU, ha, Gd, pK, VV, Si, DK, byS, XY, bK, K, Jc, gC, da, cu, jb, bN, hq, Oh, FT, HP, Gs, Gp, jd, bhP); #c1(GABRA3) c2(NP_000799) c3(gamma-aminobutyric acid receptor subunit alpha-3 precursor) c4(16189) c5(16190, 16191, 16192, 16193, 16194, 16195, 16196, 16197, 16198, 16199, 16200, 16201, 16202, 16203) c6( )c7(IJ, cA, xz, ha, hr, byT, bO, da, bS, bK, uu, V, bN, cv); #c1(GABRA3) c2(XP_005274716) c3(gamma-aminobutyric acid receptor subunit alpha-3 isoform X1) c4(285337) c5( )c6( )c7(IJ, cA, xz, ha, hr, byT, bO, da, bS, bK, uu, V, bN, cv); #c1(GABRA3) c2(XP_011529436) c3(gamma-aminobutyric acid receptor subunit alpha-3 isoform X4) c4( )c5(324875, 324876, 324877, 324878, 324879, 324880, 324881) c6( )c7(IJ, cA, xz, ha, hr, byT, bO, da, bS, bK, uu, V, bN, cv); #c1(GABRA4) c2(NP_000800) c3(gamma-aminobutyric acid receptor subunit alpha-4 isoform 1 precursor) c4(16204) c5(16205, 16206, 16207, 16208, 16209, 16210) c6( )c7(yL, ha, XY, bK, cB, bN, cv); #c1(GABRA5) c2(XP_005268315) c3(gamma-aminobutyric acid receptor subunit alpha-5 isoform X1) c4(281697, 281698, 281699, 281700, 281701) c5(281702, 281703, 281704, 281705, 281706, 281707, 281708, 281709, 281710, 281711, 28/712, 281713, 281714, 281715, 281716, 281717, 281718, 281719, 281720, 281721, 281722, 281723, 281724, 281725, 281726, 281727, 281728, 281729, 281730, 281731, 281732, 281733, 281734, 281735, 281736, 281737, 281738) c6( )c7(Na, mU, rc, jf, ot, mW, mZ, ha, bO, HN, agl, mS, mY, bK, ur, mV, mT, bN, mX); #c1(GABRA6) c2(NP_000802) c3(gamma-aminobutyric acid receptor subunit alpha-6 precursor) c4(16211, 16212) c5(16213, 16214, 16215, 16216, 16217, 16218, 16219, 16220, 16221, 16222, 16223, 16224) c6( )c7(vf, oe, ha, bS, v, ct, kl, tg, aqk, vg, bK, K, bds, da, ail, Z, jb, bN, hq, pH, Gs, sv, pk); #c1(GABRB1) c2(NP_000803) c3(gamma-aminobutyric acid receptor subunit beta-1 precursor) c4(16225, 16226) c5(16227, 16228, 16229, 16230, 16231, 16232) c6( )c7(DK, HP, ha, Gs, aPf, aga, ww, bk, il, mY, bN); #c1(GABRB1) c2(XP_011511980) c3(gamma-aminobutyric acid receptor subunit beta-1 isoform X1) c4( )c5(302638, 302639, 302640, 302641, 302642, 302643) c6( )c7(DK, HP, ha, Gs, aPf, aga, ww, bK, il, mY, bN); #c1(GABRB2) c2(NP_000804) c3(gamma-aminobutyric acid receptor subunit beta-2 isoform 2 precursor) c4(16233, 16234, 16235) c5(16236, 16237, 16238, 16239, 16240, 16241, 16242, 16243, 16244, 16245, 16246, 16247, 16248, 16249, 16250, 16251, 16252, 16253, 16254, 16255, 16256, 16257) c6( )c7(bhB, cA, ha, Gs, aqk, gl, ti, uJ, bK, kL, il, cd, agZ, bN, cv); #c1(GABRB2) c2(NP_068711) c3(gamma-aminobutyric acid receptor subunit beta-2 isoform 1 precursor) c4(210094) c5(210095, 210096, 210097, 210098, 210099, 210100, 210101, 210102, 210103, 210104) c6( )c7(bhB, cA, ha, Gs, aqk, gl, ti, uJ, bK, kL, il, cd, agZ, bN, cv); #c1(GABRB3) c2(NP_000805) c3(gamma-aminobutyric acid receptor subunit beta-3 isoform 1 precursor) c4(16258) c5(16259, 16260, 16261, 16262, 16263, 16264) c6( )c7(aC, ha, iD, bS, ct, il, rc, Na, amJ, Wk, aU, Nc, lm, bK, hg, rz, Cr, na, VX, Az, Z, byU, bN, hq, Qi, Cq, apM, agl, HN, Ei, Km, B); #c1(GABRB3) c2(NP_001178249) c3(gamma-aminobutyric acid receptor subunit beta-3 isoform 3) c4(73280) c5(73281, 73282, 73283, 73284, 73285, 73286, 73287, 73288, 73289, 73290) c6( )c7(aC, ha, iD, bS, ct, il, rc, Na, amJ, Wk, aU, Nc, lm, bk, hg, rz, Cr, na, VX, Az, Z, byU, bN, hq, Qi, Cq, apM, agl, HN, Ei, Km, B); #c1(GABRB3) c2(NP_001178250) c3(gamma-aminobutyric acid receptor subunit beta-3 isoform 4) c4(73291, 73292) c5(73293, 73294, 73295, 73296, 73297, 73298, 73299, 73300) c6( )c7(aC, ha, iD, bS, ct, il, rc, Na, amJ, Wk, aU, Nc, lm, bK, hg, rz, Cr, na, VX, Az, Z, byl, bN, hq, Qi, Cq, apM, agl, HN, Ei, Km, B); #c1(GABRB3) c2(NP_068712) c3(gamma-aminobutyric acid receptor subunit beta-3 isoform 2 precursor) c4(210105) c5(210106, 210107, 210108, 210109, 210110, 210111) c6( ) c7(aC, ha, iD, bS, ct, il, rc, Na, amJ, Wk, aU, Nc, lm, bk, hg, rz, Cr, na, VX, Az, Z, byl, bN, hq, Qi, Cq, apM, agl, HN, Ei, Km, B); #c1(GABRE) c2(NP_004952) c3(gamma-aminobutyric acid receptor subunit epsilon precursor) c4(151419) c5(151420, 151421, 151422, 151423, 151424, 151425) c6( )c7(T, Gt, XY, n, ur, bK, vo, ae, ad); #c1(GABRE) c2(XP_011529440) c3(gamma-aminobutyric acid receptor subunit epsilon isoform X4) c4(324882) c5(324883, 324884, 324885, 324886, 324887) c6( )c7(T, Gt, XY, n, ur, bK, vo, ae, ad); #c1 (GABRE) c2(XP_011529441) c3(gamma-aminobutyric acid receptor subunit epsilon isoform X5) c4( )c5(324888) c6( )c7(T, Gt, XY, n, ur, bK, vo, ae, ad); #c1(GABRG1) c2(NP_775807) c3(gamma-aminobutyric acid receptor subunit gamma-1 precursor) c4(250348, 250349) c5(250350, 250351, 250352, 250353, 250354, 250355, 250356, 250357, 250358, 250359, 250360, 250361) c6( )c7(os, dY, bre, oe, Gd, uJ, oZ, bK, bN); #c1(GABRG2) c2(NP_000807) c3(gamma-aminobutyric acid receptor subunit gamma-2 isoform 2 precursor) c4(16265, 16266) c5(16267, 16268, 16269, 16270, 16271, 16272, 16273, 16274, 16275, 16276, 16277, 16278) c6( )c7(byX, ha, al, iD, byV, bkd, kL, aqk, Wk, aU, bK, hg, xa, yr, PT, gl, cu, da, aPf, byW, hq, Cq, ok, byY, cA, Gs, HN); #c1(GABRG2) c2(NP_944493) c3(gamma-aminobutyric acid receptor subunit gamma-2 isoform 3 precursor) c4(260635) c5(260636, 260637, 260638, 260639, 260640, 260641) c6( ) c7(byX, ha, al, iD, byV, bkd, kL, aqk, Wk, aU, bK, hg, xa, yr, PT, gl, cu, da, aPf, byW, hq, Cq, ok, byY, cA, Gs, HN); #c1(GABRG2) c2(NP_944494) c3(gamma-aminobutyric acid receptor subunit gamma-2 isoform 1 precursor) c4(260642) c5(260643, 260644, 260645, 260646, 260647, 260648) c6( )c7(byX, ha, al, iD, byV, bkd, kL, aqk, Wk, aU, bK, hg, xa, yr, PT, gl, cu, da, aPf, byW, hq, Cq, ok, byY, CA, Gs, HN); #c1(GABRG3) c2(NP_001257802) c3(gamma-aminobutyric acid receptor subunit gamma-3 isoform 2 precursor) c4(95639) c5(95640, 95641, 95642, 95643, 95644, 95645) c6( )c7(bhB, agl, gl, bS, bk, UE, an, he, rc, bN, cv); #c1(GABRG3) c2(NP_150092) c3(gamma-aminobutyric acid receptor subunit gamma-3 isoform 1 precursor) c4(227739) c5(227740, 227741, 227742, 227743, 227744, 227745) c6( )c7(bhB, agl, gl, bS, bK, UE, an, he, rc, bN, cv); #c1(GABRP) c2(XP_011532807) c3(gamma-aminobutyric acid receptor subunit pi isoform X1) c4(328866, 328867, 328868, 328869, 328870, 328871) c5(328872, 328873, 328874, 328875, 328876, 328877, 328878, 328879, 328880, 328881) c6( )c7(qw, T, lg, qt, aW, aT, bS, bK, cd); #c1(GABRP) c2(XP_011532808) c3(gamma-aminobutyric acid receptor subunit pi isoform X2) c4(328882) c5(328883, 328884, 328885, 328886, 328887, 328888, 328889, 328890, 328891, 328892, 328893, 328894, 328895, 328896, 328897, 328898, 328899, 328900, 328901, 328902, 328903, 328904, 328905, 328906, 328907, 328908, 328909, 328910, 328911, 328912, 328913, 328914, 328915, 328916, 328917, 328918) c6( ) c7(qw, T, lg, qt, aW, aT, bS, bK, cd); #c1(GABRA) c2(NP_061028) c3(gamma-aminobutyric acid receptor subunit theta precursor) c4(199064, 199065) c5(199066, 199067, 199068, 199069, 199070, 199071) c6( )c7(bM, Gt, eL, aq, dL, bK, uu, ur, byl, vo, bz, B); #c1(GABRQ) c2(XP_011529486) c3(gamma-aminobutyric acid receptor subunit theta isoform X1) c4( )c5(324950, 324951, 324952, 324953, 324954, 324955) c6( )c7(bM, Gt, eL, aq, dl, bK, uu, ur, byl, vo, bz, B); #c1(GABRR1) c2(NP_001243632) c3(gamma-aminobutyric acid receptor subunit rho-I isoform b precursor) c4(91266) c5(91267, 91268, 91269, 91270, 91271, 91272) c6( )c7(Gt, iH, bK); #c1 (GABRR1) c2(NP_002033) c3(gamma-aminobutyric acid receptor subunit rho-I isoform a precursor) c4(123493) c5(123494, 123495, 123496, 123497, 123498, 123499, 123500) c6( )c7(Gt, iH, bK); #c1(GABRR1) c2(XP_006715501) c3(gamma-aminobutyric acid receptor subunit rho-1 isoform X2) c4( )c5(289601) c6( )c7(Gt, iH, bK); #c1(GABRR1) c2(XP_011534014) c3 (gamma-aminobutyric acid receptor subunit rho-1 isoform X1) c4(330186) c5(330187, 330188, 330189, 330190, 330191, 330192, 330193, 330194) c6( )c7(Gt, iH, bk); #c1(GABRR2) c2(NP_002034) c3(gamma-aminobutyric acid receptor subunit rho-2 precursor) c4(123501) c5(123502, 123503, 123504, 123505, 123506, 123507) c6( )c7(Gt, nX, ha, hl, bk, byl, iq); #c1(GAD1) c2(NP_000808) c3(glutamate decarboxylase 1 isoform GAD67) c4(16279, 16280, 16281, 16282, 16283) c5(16284, 16285, 16286, 16287, 16288, 16289) c6( )c7(bL, eW, cZ, T, LX, yG, ha, aFc, bO, mS, n, byZ, bS, ct, ti, ci, fu, dG, V, bX, j, J, gF, aR, ad, lF, Fb, mj, F, aE, ww, HJ, Na, mW, bza, hD, Nc, bK, K, dd, an, mY, mV, bhm, he, ae, rz, rE, mU, A, tb, eZ, zD, Mz, gl, cu, Z, FX, kk, fr, mZ, jb, rY, mX, cv, hq, pH, aj, bN, ky, cA, Lj, wD, SC, yP, U, mg, sv, mf, uJ, avD, v, xg, mT, qc); #c1(GAD1) c2(NP_038473) c3(glutamate decarboxylase 1 isoform GAD25) c4(176613) c5(176614, 176615, 176616, 176617, 176618, 176619, 176620, 176621, 176622, 176623, 176624, 176625, 176626, 176627, 176628, 176629, 176630, 176631, 176632, 176633, 176634, 176635, 176636, 176637, 176638, 176639, 176640, 176641, 176642, 176643, 176644, 176645, 176646, 176647) c6( )c7(bL, eW, cZ, T, LX, yG, ha, aFc, bO, mS, n, byZ, bS, ct, ti, ci, fu, dG, V, bX, j, J, gF, aR, ad, lF, Fb, mj, F, aE, ww, HJ, Na, mW, bza, hD, Nc, bK, K, dd, an, mY, mV, bhm, he, ae, rz, rE, mU, A, tb, eZ, zD, Mz, gl, cu, Z, FX, kk, fr, mZ, jb, rY, mX, cv, hq, pH, aj, bN, ky, cA, Lj, wD, SC, yP, U, mg, sv, mf, uJ, avD, v, xg, mT, qc); #c1(GAD2) c2(NP_001127838) c3(glutamate decarboxylase 2) c4(49308, 49309, 49310) c5(49311, 49312, 49313, 49314, 49315, 49316, 49317, 49318, 49319, 49320, 49321, 49322, 49323, 49324, 49325, 49326, 49327, 49328, 49329, 49330, 49331, 49332) c6( )c7(bL, cZ, dD, LX, yG, qQ, aFc, bzb, bO, ti, aj, v, j, hz, Ea, eq, ww, J, lF, aqt, mj, cF, aE, hD, hg, uV, dd, qM, an, bhm, af, uX, A, lR, tb, s, Mz, dq, cu, gu, Z, fq, rY, hq, bN, pH, ky, aDX, cA, bo, yP, aiM, mg, vy, uJ, zB); #c1(GADD45B) c2(NP_056490) c3(growth arrest and DNA damage-inducible protein GADD45 beta) c4(187736) c5(187737, 187738, 187739, 187740, 187741, 187742) c6( )c7(gx, T, ht, jx, bB, ai, q, cc, cq, hW, Dj, K, lQ, hu, dF, aiJ, Dg, gl, aq, pl, iR, zr, bE, m, Pk, Pr, B, y); #c1(GADD45G) c2(NP_006696) c3(growth arrest and DNA damage-inducible protein GADD45 gamma) c4(167213, 167214) c5(167215, 167216, 167217, 167218, 167219, 167220, 167221, 167222, 167223, 167224, 167225, 167226) c6( )c7(T, os, aw, ht, aA, it, n, KO, oZ, uE, ad, av, pN, aT, rO, K, hu, ae, WW, aa, apl, yo, s, aW, bz, ac, pP, hR, dy, m, Pr, B); #c1(GADD45GIP1) c2(NP_443082) c3(growth arrest and DNA damage-inducible proteins-interacting protein 1) c4(229315) c5(229316, 229317, 229318, 229319, 229320, 229321, 229322) c6( )c7(ck, bt, ae, ad); #c1(GADL1) c2(NP_997242) c3(acidic amino acid decarboxylase GADL1) c4(265261) c5(265262, 265263, 265264, 265265, 265266, 265267) c6( )c7(amN, av, Qy, d, eM, Tf, Y, ff, aq, amM, m, bz, en, oi, t, g, fq, as, Hr); #c1 (GAGE1) c2(NP_001035753) c3(G antigen 1) c4(33031) c5(33032, 33033, 33034, 33035, 33036, 33037) c6( )c7(Y, aC, B, g, bu); #c1(GAGE10) c2(NP_001091883) c3(G antigen 10) c4(38782) c5(38783, 38784, 38785, 38786, 38787, 38788) c6( )c7(T); #c1(GAK) c2(NP_005246) c3(cyclin-G-associated kinase isoform 1) c4(154371, 154372) c5(154373, 154374, 154375, 154376, 154377, 154378) c6( ) c7(LS, an); #c1(GAL) c2(NP_057057) c3(galanin peptides preproprotein) c4(188673) c5(188674, 188675, 188676, 188677, 188678, 188679) c6( )c7(cZ, T, Tb, e, ha, aw, bO, cz, bB, aj, bS, uu, ct, C, ci, bze, v, il, fr, q, J, eA, eW, bjV, ft, ww, uT, il, sO, acu, bzd, aT, cu, Ps, bK, K, wb, dd, bq, dQ, Ha, af, eu, hZ, eG, Uf, d, yo, tb, A, aW, ev, Gv, ff, r, lo, en, Z, cm, ik, bzc, FT, hq, ok, cA, dB, bE, eD, Gs, hO, hR, Hs, Lq, m, jd, B, hS); #c1(GALC) c2(NP_000144) c3(galactocerebrosidase isoform a precursor) c4(3096, 3097) c5(3098, 3099, 3100, 3101, 3102, 3103, 3104) c6( )c7(bzg, cb, dH, ff, U, dq, aYR, Lq, aAt, en, mF, bzf, aR); #c1(GALC) c2(NP_001188330) c3(galactocerebrosidase isoform c precursor) c4(79069) c5(79070, 79071, 79072, 79073, 79074, 79075, 79076) c6( )c7(bzg, cb, dH, ff, U, dq, aYR, Lq, aAt, en, mF, bzf, aR); #c1 (GALC) c2(NP_001188331) c3(galactocerebrosidase isoform d) c4(79077) c5(79078, 79079, 79080, 79081, 79082, 79083, 79084) c6( )c7(bzg, cb, dH, ff, U, dq, aYR, Lq, aAt, en, mF, bzf, aR); #c1(GALC) c2(XP_011534920) c3(galactocerebrosidase isoform X1) c4(331494) c5(331495, 331496, 331497, 331498, 331499, 331500, 331501, 331502, 331503, 331504, 331505, 331506) c6( )c7(bzg, cb, dH, ff, U, dq, aYR, Lq, aAt, en, mF, bzf, aR); #c1(GALK1) c2(NP_000145) c3(galactokinase) c4(3105, 3106, 3107) c5(3108, 3109, 3110, 3111, 3112, 3113, 3114, 3115, 3116, 3117, 3118, 3119, 3120, 3121, 3122, 3123, 3124, 3125, 3126, 3127, 3128, 3129, 3130, 3131, 3132, 3133, 3134, 3135, 3136, 3137, 3138, 3139, 3140, 3141, 3142, 3143, 3144, 3145, 3146, 3147, 3148, 3149, 3150, 3151, 3152, 3153, 3154, 3155, 3156, 3157, 3158, 3159, 3160, 3161, 3162, 3163, 3164, 3165, 3166, 3167, 3168, 3169, 3170, 3171, 3172, 3173, 3174, 3175, 3176, 3177, 3178, 3179, 3180, 3181, 3182, 3183, 3184, 3185, 3186, 3187, 3188, 3189, 3190, 3191, 3192, 3193, 3194, 3195, 3196, 3197, 3198, 3199, 3200, 3201, 3202, 3203, 3204, 3205, 3206, 3207, 3208, 3209, 3210, 3211, 3212, 3213, 3214, 3215, 3216) c6(3217) c7(mO, bzj, aWG, bzi, r, QT, bzh); #c1(GALK2) c2(NP_001001556) c3(N-acetylgalactosamine kinase isoform 2) c4(18712) c5(18713, 18714, 18715, 18716, 18717, 18718, 18719, 18720, 18721) c6( )c7(ey); #c1(GALK2) c2(NP_001275959) c3 (N-acetylgalactosamine kinase isoform 3) c4( )c5(107533, 107534, 107535, 107536, 107537, 107538, 107539, 107540, 107541, 107542, 107543) c6( )c7(ey); #c1(GALK2) c2(NP_002035) c3(N-acetylgalactosamine kinase isoform 1) c4(123508, 123509, 123510) c5(123511, 123512, 123513, 123514, 123515, 123516) c6( )c7(ey); #c1(GALK2) c2(XP_005254336) c3(N-acetylgalactosamine kinase isoform X1) c4( )c5(273159) c6( )c7(ey); #c1(GALK2) c2(XP_011519742) c3(N-acetylgalactosamine kinase isoform X8) c4( )c5(314431, 314432, 314433, 314434, 314435) c6( )c7(ey); #c1(GALM) c2(NP_620156) c3(aldose 1-epimerase) c4(237381) c5(237382, 237383, 237384, 237385, 237386, 237387) c6( )c7(r, fe, bak, iV, q, dr); #c1(GALNS) c2(NP_000503) c3(N-acetylgalactosamine-6-sulfatase precursor) c4(10462, 10463) c5(10464, 10465, 10466, 10467, 10468, 10469, 10470, 10471, 10472, 10473, 10474, 10475, 10476, 10477, 10478, 10479, 10480, 10481, 10482, 10483, 10484, 10485, 10486, 10487, 10488, 10489, 10490, 10491, 10492, 10493, 10494, 10495, 10496, 10497, 10498, 10499, 10500, 10501, 10502, 10503, 10504, 10505, 10506, 10507, 10508, 10509, 10510, 10511, 10512, 10513, 10514, 10515, 10516, 10517, 10518, 10519, 10520, 10521, 10522, 10523, 10524, 10525, 10526, 10527, 10528, 10529, 10530, 10531, 10532, 10533, 10534, 10535, 10536, 10537, 10538, 10539, 10540, 10541, 10542, 10543, 10544, 10545, 10546, 10547, 10548, 10549, 10550, 10551, 10552, 10553, 10554, 10555, 10556, 10557, 10558, 10559, 10560, 10561, 10562, 10563, 10564, 10565, 10566, 10567, 10568, 10569, 10570, 10571, 10572) c6(10573, 10574, 10575, 10576, 10577) c7(aA, ade, C, auu, bjQ, Bl, pN, bzk, aT, h, vO, adn, bA, aW, dq, aF, afs, bzl, W, kg, qr, cj, Zn, bzm, acZ, Lq, azy, bH); #c1(GALNS) c2(XP_005256358) c3(N-acetylgalactosamine-6-sulfatase isoform X2) c4(274127) c5(274128, 274129, 274130) c6(274131) c7(aA, ade, C, auu, bjQ, Bl, pN, bzk, aT, h, vO, adn, bA, aW, dq, aF, afs, bzl, W, kg, qr, cj, Zn, bzm, acZ, Lq, azy, bH); #c1 (GALNT1) c2(XP_005258296) c3(polypeptide N-acetylgalactosaminyltransferase 1 isoform X1) c4(275608, 275609) c5(275610, 275611, 275612, 275613, 275614, 275615, 275616, 275617, 275618, 275619, 275620, 275621, 275622, 275623, 275624, 275625) c6( )c7(t, ey, m, c); #c1(GALNT12) c2(NP_078918) c3(polypeptide N-acetylgalactosaminyltransferase (2) c4(216117) c5(216118, 216119, 216120, 216121, 216122, 216123) c6( )c7(T, ac, r, q, X, bzn); #c1(GALNT12) c2(XP_006717350) c3(polypeptide N-acetylgalactosaminyltransferase 12 isoform X4) c4( )c5(290962) c6( )c7(T, ac, r, q, X, bzn); #c1(GALNT13) c2(NP_001288556) c3(polypeptide N-acetylgalactosaminyltransferase 13 isoform 2) c4(113749) c5(113750, 113751, 113752, 113753, 113754, 113755) c6( )c7(qb, ff, ht, gv, j, en); #c1(GALNT13) c2(NP_443149) c3(polypeptide N-acetylgalactosaminyltransferase 13 isoform 1) c4(229647, 229648) c5(229649, 229650, 229651, 229652, 229653, 229654, 229655) c6( )c7(qb, ff, ht, gv, j, en); #c1(GALNT13) c2(XP_011508839) c3(polypeptide N-acetylgalactosaminyltransferase 13 isoform X1) c4( )c5(299425, 299426, 299427, 299428, 299429) c6( )c7(qb, ff, ht, gv, j, en); #c1(GALNT14) c2(NP_001240755) c3(polypeptide N-acetylgalactosaminyltransferase 14 isoform 2) c4(88756) c5(88757, 88758, 88759, 88760, 88761, 88762) c6( )c7(zo, T, B, Y); #c1(GALNT14) c2(NP_078848) c3(polypeptide N-acetylgalactosaminyltransferase 14 isoform 1) c4(215696, 215697) c5(215698, 215699, 215700, 215701, 215702, 215703, 215704, 215705, 215706, 215707, 215708, 215709) c6( )c7(zo, T, B, Y); #c1(GALNT14) c2(XP_011531406) c3 (polypeptide N-acetylgalactosaminyltransferase 14 isoform X2) c4(327479) c5(327480, 327481, 327482, 327483, 327484, 327485) c6( )c7(zo, T, B, Y); #c1(GALNT14) c2(XP_011531407) c3(polypeptide N-acetylgalactosaminyltransferase 14 isoform X1) c4(327486) c5(327487, 327488, 327489, 327490, 327491, 327492) c6( )c7(zo, T, B, Y); #c1(GALNT15) c2(NP_473451) c3(polypeptide N-acetylgalactosaminyltransferase 15) c4(230539, 230540) c5(230541, 230542, 230543, 230544, 230545, 230546, 230547, 230548, 230549, 230550, 230551, 230552) c6( )c7(qb, if, q, qN, r); #c1(GALNT18) c2(NP_940918) c3(polypeptide N-acetylgalactosaminyltransferase 18) c4(260110) c5(260111, 260112, 260113, 260114, 260115, 260116) c6( )c7(y, fq, ad); #c1(GALNT2) c2(NP_001278795) c3(polypeptide N-acetylgalactosaminyltransferase 2 isoform 2) c4(110123, 110124) c5(110125, 110126, 110127, 110128, 110129, 110130) c6( )c7(bL, Zu, ac, c0, ff, cz, n, dG, j, ci, v, ei, en, B, eu); #c1(GALNT2) c2(NP_004472) c3(polypeptide N-acetylgalactosaminyltransferase 2 precursor) c4(146656, 146657) c5(146658, 146659, 146660, 146661, 146662, 146663, 146664) c6( )c7(bL, Zu, ac, c0, ff, cz, n, dG, j, ci, v, ei, en, B, eu); #c1(GALNT2) c2(XP_011542457) c3 (polypeptide N-acetylgalactosaminyltransferase 2 isoform X2) c4( )c5(340955, 340956, 340957, 340958, 340959, 340960, 340961, 340962, 340963, 340964, 340965) c6( )c7(bL, Zu, ac, c0, ff, cz, n, dG, j, ci, v, ei, en, B, eu); #c1(GALNT3) c2(NP_004473) c3(polypeptide N-acetylgalactosaminyltransferase 3) c4(146665, 146666) c5(146667, 146668, 146669, 146670, 146671, 146672, 146673, 146674, 146675, 146676, 146677, 146678, 146679, 146680, 146681, 146682, 146683, 146684, 146685, 146686, 146687, 146688) c6( )c7(n, aj, fr, ci, J, ad, av, aE, io, bta, bsR, hB, ae, bA, aDy, A, ff, dq, bzo, en, g, akb, t, cM, bz, alx, oX, lS, m, iu); #c1(GALNT3) c2(XP_006712465) c3(polypeptide N-acetylgalactosaminyltransferase 3 isoform X2) c4(286667, 286668, 286669) c5(286670, 286671, 286672, 286673, 286674, 286675, 286676, 286677, 286678, 286679, 286680, 286681, 286682) c6( )c7(n, aj, fr, ci, J, ad, av, aE, io, bta, bsR, hB, ae, bA, aly, A, ff, dq, bzo, en, g, akb, t, cM, bz, alx, oX, lS, m, iu); #c1(GALNT4) c2(NP_003765) c3(polypeptide N-acetylgalactosaminyltransferase 4) c4(140664) c5(140665, 140666, 140667, 140668, 140669, 140670) c6( )c7(j); #c1(GALNT5) c2(NP_055383) c3(polypeptide N-acetylgalactosaminyltransferase 5) c4(180705) c5(180706, 180707, 180708, 180709, 180710, 180711) c6( )c7(aF, aA); #c1(GALNTG) c2(XP_006719277) c3(polypeptide N-acetylgalactosaminyltransferase 6 isoform X1) c4(292370, 292371) c5(292372, 292373, 292374, 292375, 292376, 292377, 292378, 292379, 292380, 292381, 292382, 292383, 292384, 292385, 292386, 292387) c6( )c7(aW, n, T, ci, aT); #c1(GALNT7) c2(NP_059119) c3(N-acetylgalactosaminyltransferase 7) c4(193142) c5(193143, 193144, 193145, 193146, 193147, 193148) c6( )c7(if, ey, qN); #c1(GALNT7) c2(XP_005263119) c3(N-acetylgalactosaminyltransferase 7 isoform X1) c4(278320) c5(278321, 278322, 278323, 278324, 278325, 278326) c6( )c7(if, ey, qN); #c1(GALNT8) c2(NP_059113) c3(probable polypeptide N-acetylgalactosaminyltransferase 8) c4(193106) c5(193107, 193108, 193109, 193110, 193111, 193112) c6( )c7(F); #c1(GALNT9) c2(NP_001116108) c3(polypeptide N-acetylgalactosaminyltransferase 9 isoform A) c4(43603, 43604, 43605, 43606, 43607) c5(43608, 43609, 43610, 43611, 43612, 43613, 43614, 43615, 43616, 43617, 43618, 43619, 43620, 43621, 43622, 43623, 43624, 43625, 43626) c6( ) c7(cm); #c1(GALNT9) c2(NP_068580) c3(polypeptide N-acetylgalactosaminyltransferase 9 isoform B) c4( )c5(209689, 209690, 209691, 209692, 209693, 209694, 209695, 209696, 209697, 209698, 209699, 209700, 209701, 209702, 209703, 209704, 209705, 209706, 209707, 209708, 209709) c6( )c7(cm); #c1(GALNTL6) c2(NP_001030017) c3(polypeptide N-acetylgalactosaminyltransferase-like 6) c4(30860, 30861) c5(30862, 30863, 30864, 30865, 30866, 30867, 30868, 30869, 30870, 30871, 30872, 30873) c6( )c7(fe, Ea, oe, fq); #c1(GALP) c2(NP_001139018) c3(galanin-like peptide isoform 2 precursor) c4(58617) c5(58618, 58619, 58620, 58621, 58622, 58623) c6(58624) c7(Tg, v, af, Tb, bq); #c1(GALP) c2(NP_149097) c3(galanin-like peptide isoform 1 precursor) c4(227131) c5(227132, 227133, 227134, 227135, 227136, 227137, 227138, 227139, 227140) c6(227141, 227142) c7(Tg, v, af, Tb, bq); #c1(GALR1) c2(NP_001471) c3(galanin receptor type 1) c4(117987) c5(117988, 117989, 117990, 117991, 117992, 117993) c6( )c7(d, Gv, ff, EQ, r, hR, v, en, he, ik, q); #c1(GALR2) c2(NP_003848) c3(galanin receptor type 2) c4(141404) c5(141405, 141406, 141407, 141408, 141409, 141410) c6( ) c7(T, ft, d, bS, wb, K, ct, Z, cm, ik, hq); #c1(GALR3) c2(XP_011528770) c3(galanin receptor type 3 isoform X1) c4(324077) c5(324078, 324079, 324080, 324081, 324082, 324083) c6( )c7(hR, he, cF, wb); #c1(GALT) c2(NP_000146) c3(galactose-1-phosphate uridylyltransferase isoform 1) c4(3218, 3219) c5(3220, 3221, 3222, 3223, 3224, 3225, 3226, 3227, 3228, 3229, 3230, 3231, 3232, 3233, 3234, 3235, 3236, 3237, 3238, 3239, 3240, 3241, 3242, 3243, 3244, 3245, 3246, 3247, 3248, 3249, 3250, 3251, 3252, 3253, 3254, 3255, 3256, 3257, 3258, 3259, 3260, 3261, 3262, 3263, 3264, 3265, 3266, 3267, 3268, 3269, 3270, 3271, 3272, 3273, 3274, 3275, 3276, 3277, 3278, 3279, 3280, 3281, 3282, 3283, 3284, 3285, 3286, 3287, 3288, 3289, 3290, 3291, 3292, 3293, 3294, 3295, 3296, 3297, 3298, 3299, 3300, 3301, 3302, 3303, 3304, 3305, 3306, 3307, 3308, 3309, 3310, 3311, 3312, 3313, 3314, 3315, 3316, 3317, 3318, 3319, 3320, 3321, 3322, 3323, 3324, 3325, 3326, 3327, 3328, 3329, 3330, 3331, 3332, 3333, 3334, 3335, 3336, 3337, 3338, 3339, 3340, 3341, 3342, 3343, 3344, 3345, 3346, 3347, 3348, 3349, 3350, 3351, 3352, 3353, 3354, 3355, 3356, 3357, 3358, 3359, 3360, 3361, 3362, 3363, 3364, 3365, 3366, 3367, 3368) c6(3369) c7(dP, bfP, mO, b, zZ, il, WQ, blA, Ps, dq, aj, CZ, h, m, aTS, i, t, dr, anU); #c1(GALT) c2(NP_001245261) c3(galactose-l-phosphate uridylyltransferase isoform 2) c4(92944) c5(92945, 92946, 92947, 92948, 92949, 92950) c6( )c7(dP, bfP, mO, b, zZ, il, WQ, blA, Ps, dq, aj, CZ, h, m, aTS, i, t, dr, anU); #c1(GAMT) c2(NP_000147) c3(guanidinoacetate N-methyltransferase isoform a) c4(3370) c5(3371, 3372, 3373, 3374, 3375, 3376) c6( )c7(nc, lp, M, bzp, nA, rL); #c1(GAMT) c2(NP_620279) c3(guanidinoacetate N-methyltransferase isoform b) c4(237500) c5(237501, 237502, 237503, 237504, 237505, 237506) c6( )c7(nc, lp, M, bzp, nA, rL); #c1(GAN) c2(NP_071324) c3(gigaxonin) c4(210976) c5(210977, 210978, 210979, 210980, 210981, 210982) c6( )c7(T, U, bk, aTe, fu, bU, bX, Kq, aR); #c1(GANAB) c2(NP_001265123) c3(neutral alpha-glucosidase AB isoform 6) c4( )c5(97815, 97816, 97817, 97818, 97819) c6( )c7(T); #c1(GANAB) c2(NP_938148) c3(neutral alpha-glucosidase AB isoform 2 precursor) c4(259624, 259625) c5(259626, 259627, 259628, 259629, 259630, 259631, 259632) c6( )c7(T); #c1(GANAB) c2(NP_938149) c3(neutral alpha-glucosidase AB isoform 3 precursor) c4(259633) c5(259634, 259635, 259636, 259637, 259638, 259639) c6( )c7(T); #c1(GANC) c2(NP_001288339) c3(neutral alpha-glucosidase C isoform 3) c4(113737, 113738) c5(113739, 113740, 113741, 113742, 113743, 113744, 113745, 113746, 113747, 113748) c6( )c7(adw); #c1(GANC) c2(NP_937784) c3(neutral alpha-glucosidase C isoform 1) c4(259076) c5(259077, 259078, 259079, 259080, 259081, 259082) c6( )c7(adw); #c1(GAP43) c2(NP_001123536) c3(neuromodulin isoform 1) c4(47442) c5(47443, 47444, 47445, 47446, 47447, 47448) c6( )c7(amt, xw, sS, bb, c, Ol, qr, jx, ti, cd, bS, ct, aUO, Z, cm, V, cB, X, aR, rY, L); #c1(GAP43) c2(NP_002036) c3(neuromodulin isoform 2) c4(123517) c5(123518, 123519, 123520, 123521, 123522, 123523) c6( )c7(amt, xw, sS, bb, c, Ol, qr, jx, ti, cd, bS, ct, aUO, Z, cm, V, cB, X, aR, rY, L); #c1(GAPDH) c2(NP 001243728) c3(glyceraldehyde-3-phosphate dehydrogenase isoform 2) c4(91598, 91599) c5(91600, 91601, 91602, 91603, 91604, 91605, 91606, 91607, 91608, 91609) c6( )c7(bM, T, Tb, if, al, eb, fd, Ja, lR, ci, Ul, ip, aE, K, zy, eL, aW, hl, pD, g, as, ef, cm, eM, nB, aeB, wK, mx, U, vy, iS, nw, m, lT, wu, ht, n, cd, aj, fr, qN, fK, BI, cc, h, nm, dL, ey, ox, bzs, bzr, fe, aq, mo, hR, sZ, B, it, bL, fM, AS, AL, Ju, jx, aA, aBo, v, j, V, pd, q, J, eA, eJ, mP, aT, vH, cy, ae, af, bA, s, Y, r, en, t, f, ik, pb, ck, rR, c, VA, bt, yv, pY, qb, iW, jh, bzq, OH, ad, av, Tg, hd, db, vJ, ds, wm, Od, nJ, d, ff, wc, eH, cr, yy, aF, iR, bz, es, gk, ll, aS, cP, y, X); #c1(GAPDH) c2(NP_001276674) c3(glyceraldehyde-3-phosphate dehydrogenase isoform 1) c4(108225, 108226) c5(108227, 108228, 108229, 108230, 108231, 108232, 108233, 108234, 108235, 108236, 108237, 108238, 108239, 108240, 108241, 108242, 108243, 108244, 108245, 108246, 108247, 108248) c6( )c7(bM, T, Tb, if, al, eb, fd, Ja, lR, ci, Ul, ip, aE, K, zy, eL, aW, hl, pD, g, as, eF, cm, eM, nB, aeB, wK, mx, U, vy, iS, nw, m, lT, wu, ht, n, cd, aj, fr, qN, fK, BI, cc, h, nm, dL, ey, ox, bzs, bzr, fe, aq, mo, hR, sZ, B, it, bL, M, AS, AL, Ju, jx, aA, aBo, v, j, V, pd, q, J, eA, eJ, mP, aT, vH, cy, ae, af, bA, s, Y, r, en, t, f, iK, pb, ck, rR, c, VA, bt, yv, pY, qb, iW, jh, bzq, OH, ad, av, Tg, hd, db, vJ, ds, wm, Od, nJ, d, ff, wc, eH, cr, yy, aF, iR, bz, es, gk, ll, aS, cP, y, X); #c1(GAPDHS) c2(NP_055179) c3(glyceraldehyde-3-phosphate dehydrogenase, testis-specific) c4(179605) c5(179606, 179607, 179608, 179609, 179610, 179611) c6( )c7(af); #c1(GAR1) c2(NP_061856) c3(H/ACA ribonucleoprotein complex subunit 1) c4(200821) c5(200822, 200823, 200824, 200825, 200826, 200827, 200828, 200829, 200830, 200831) c6( )c7(yd, oY, dW, sb); #c1(GAREM) c2(NP_001229338) c3(GRB2-associated and regulator of MAPK protein isoform 1) c4(84233) c5(84234, 84235, 84236, 84237, 84238, 84239) c6( )c7(de); #c1(GAREM) c2(NP_073588) c3(GRB2-associated and regulator of MAPK protein isoform 2) c4(212994) c5(212995, 212996, 212997, 212998, 212999, 213000) c6( )c7(oe); #c1(GARNL3) c2(NP_001273708) c3(GTPase-activating Rap/Ran-GAP domain-like protein 3 isoform b) c4(105695) c5(105696, 105697, 105698, 105699, 105700, 105701) c6( )c7(bk); #c1(GARNL3) c2(NP_115669) c3(GTPase-activating Rap/Ran-GAP domain-like protein 3 isoform a) c4(223505, 223506) c5(223507, 223508, 223509, 223510, 223511, 223512, 223513, 223514, 223515, 223516, 223517, 223518) c6( )c7(bk); #c1(GARNL3) c2(XP_005252324) c3(GTPase-activating Rap/Ran-GAP domain-like protein 3 isoform X1) c4(271660) c5(271661) c6( )c7(bK); #c1(GARNL3) c2(XP_011517391) c3(GTPase-activating Rap/Ran-GAP domain-like protein 3 isoform X3) c4( )c5(311114, 311115, 311116, 311117, 311118, 311119, 311120, 311121, 311122) c6( )c7(bK); #c1(GARS) c2(NP_002038) c3(glycine-tRNA ligase precursor) c4(123524) c5(123525, 123526, 123527, 123528, 123529, 123530) c6( )c7(II, T, if, jh, aA, j, fu, bX, qN, ad, lF, LN, no, aE, BJ, ld, HC, aT, kf, bl, bu, ae, Oq, aW, bT, ff, lb, bhi, eH, r, aq, aF, en, LK, t, eF, Bv, Ew, ql, c, w, bk, m, B); #c1(GART) c2(NP_780294) c3(trifunctional purine biosynthetic protein adenosine-3 isoform 2) c4( )c5(251530, 251531, 251532, 251533, 251534, 251535, 251536, 251537, 251538, 251539, 251540, 251541, 251542, 251543, 251544, 251545, 251546, 251547, 251548, 251549, 251550, 251551, 251552, 251553, 251554, 251555, 251556, 251557, 251558, 251559) c6( )c7(Na, no, W, lp, r, adW, PD, Nc, K, fu, bU, LK, f, af, ad); #c1(GART) c2(XP_006724053) c3(trifunctional purine biosynthetic protein adenosine-3 isoform X1) c4(295633, 295634, 295635, 295636, 295637, 295638, 295639) c5(295640, 295641, 295642, 295643, 295644, 295645, 295646, 295647, 295648, 295649, 295650, 295651, 295652, 295653, 295654, 295655, 295656, 295657, 295658, 295659, 295660, 295661, 295662, 295663, 295664, 295665, 295666, 295667, 295668) c6( )c7(Na, no, W, lp, r, adW, PD, Nc, K, fu, bU, LK, f, af, ad); #c1(GAS1) c2(NP_002039) c3(growth arrest-specific protein 1 precursor) c4(123531) c5(123532, 123533, 123534, 123535, 123536, 123537, 123538) c6( )c7(Na, T, asp, nM, dH, db, eL, Y, r, aA, n, cy, dL, cm, eA, aF, ZW, t, ae, q, ad); #c1(GAS2) c2(NP_001137302) c3 (growth arrest-specific protein 2) c4(55401) c5(55402, 55403, 55404, 55405, 55406, 55407, 55408, 55409, 55410, 55411, 55412, 55413, 55414, 55415) c6( )c7(gZ, bL, cF, qK, il); #c1(GAS2) c2(XP_011518275) c3(growth arrest-specific protein 2 isoform X1) c4(312281, 312282, 312283, 312284) c5( )c6( ) c7(gZ, bL, cF, qK, il); #c1(GAS2) c2(XP_011518281) c3(growth arrest-specific protein 2 isoform X6) c4( )c5(312285, 312286, 312287, 312288, 312289, 312290, 312291, 312292, 312293, 312294, 312295, 312296, 312297, 312298, 312299, 312300, 312301, 312302, 312303, 312304, 312305, 312306, 312307, 312308) c6( )c7(gZ, bL, cF, qK, iU); #c1(GAS2LI) c2(NP_001265659) c3(GAS2-like protein 1 isoform c) c4( )c5(99144, 99145, 99146, 99147, 99148, 99149, 99150, 99151, 99152, 99153, 99154, 99155, 99156, 99157, 99158, 99159, 99160, 99161, 99162, 99163, 99164, 99165, 99166, 99167) c6( )c7(bt); #c1(GAS2LI) c2(NP_689422) c3(GAS2-like protein 1 isoform a) c4(242710, 242711, 242712, 242713, 242714, 242715) c5(242716, 242717, 242718, 242719, 242720, 242721, 242722, 242723, 242724, 242725, 242726, 242727, 242728, 242729, 242730, 242731, 242732, 242733, 242734, 242735, 242736, 242737, 242738, 242739) c6( )c7(bt); #c1(GAS6) c2(NP_000811) c3(growth arrest-specific protein 6 precursor) c4(16290, 16291, 16292, 16293, 16294) c5(16295, 16296, 16297, 16298, 16299, 16300, 16301, 16302, 16303, 16304, 16305, 16306, 16307, 16308, 16309, 16310, 16311, 16312, 16313, 16314, 16315, 16316, 16317, 16318, 16319, 16320, 16321, 16322, 16323, 16324) c6( )c7(bL, bM, T, dD, gU, Uc, ea, e, jh, aA, Bm, eq, fr, V, ai, ad, eA, x, oT, hd, hW, db, qK, oW, aT, dz, ji, hH, ae, eu, Qe, aW, Ls, bzu, eZ, gm, kp, aF, cl, pl, t, pt, bz, ck, bzt, W, nb, ac, hc, vA, E, cS, m, k, y, bt, iu); #c1(GAS7) c2(NP_001124303) c3(growth arrest-specific protein 7 isoform d) c4(48523, 48524) c5(48525, 48526, 48527, 48528, 48529) c6( ) c7(ck, ac, dH, Qu, r, v, ey, eA, iV, bt, q); #c1(GAS7) c2(NP_003635) c3(growth arrest-specific protein 7 isoform a) c4(139768) c5(139769, 139770, 139771, 139772, 139773, 139774, 139775, 139776, 139777, 139778, 139779, 139780, 139781, 139782, 139783, 139784, 139785, 139786, 139787, 139788, 139789) c6( )c7(ck, ac, dH, Qu, r, v, ey, eA, iV, bt, q); #c1(GAS7) c2(NP_958836) c3(growth arrest-specific protein 7 isoform b) c4(262237, 262238, 262239) c5(262240, 262241, 262242, 262243, 262244, 262245, 262246, 262247, 262248, 262249, 262250, 262251, 262252, 262253, 262254, 262255, 262256) c6( )c7(ck, ac, dH, Qu, r, v, ey, eA, iV, bt, q); #c1(GAS7) c2(NP_958839) c3(growth arrest-specific protein 7 isoform c) c4(262257) c5(262258, 262259, 262260, 262261, 262262, 262263, 262264) c6( )c7(ck, ac, dH, Qu, r, v, ey, eA, iV, bt, q); #c1(GAS8) c2(NP_001273137) c3(growth arrest-specific protein 8 isoform c) c4( ) c5(104336) c6( )c7(aW, cd, aT); #c1(GAS8) c2(NP_001273138) c3(growth arrest-specific protein 8 isoform d) c4(104337) c5(104338, 104339, 104340, 104341, 104342, 104343, 104344, 104345, 104346, 104347, 104348) c6( )c7(aW, cd, aT); #c1(GAS8) c2(NP_001472) c3(growth arrest-specific protein 8 isoform a) c4(117994, 117995, 117996) c5(117997, 117998, 117999, 118000, 118001, 118002, 118003, 118004, 118005, 118006, 118007, 118008, 118009) c6( )c7(aW, cd, aT); #c1(GAST) c2(NP_000796) c3(gastrin preproprotein) c4(16144) c5(16145, 16146, 16147, 16148, 16149, 16150) c6( )c7(T, bzx, aC, fR, atj, bB, ci, xy, aE, pX, bzw, azh, li, iE, JI, aW, az, LZ, nY, kt, aB, g, as, aoS, qr, cj, Zn, azg, m, ct, Bl, ft, Do, h, lG, Yd, ahJ, asE, bzw, W, pV, bzm, bzy, dA, aA, C, q, eA, eJ, nv, qp, aT, bau, dl, bA, jf, s, aBN, r, aNi, afs, t, ck, alt, gX, Ze, bH, On, akd, pY, aD, bS, bjQ, av, hD, yh, fb, vO, Yi, mu, d, yy, aF, Z, HI, bz, bF, bE, AR, gZ, azy, X, Ex); #c1(GATA1) c2(NP_002040) c3(erythroid transcription factor) c4(123539, 123540) c5(123541, 123542, 123543, 123544, 123545, 123546, 123547, 123548, 123549, 123550, 123551, 123552, 123553, 123554, 123555, 123556, 123557, 123558, 123559) c6( )c7(bM, T, aC, xu, bzE, alT, oA, bzA, bzC, oY, ci, hF, aYh, br, ad, er, x, CJ, fX, yw, BR, dE, oW, aT, bzB, h, dz, iY, DU, ae, af, Nr, iU, et, aW, adw, eH, KX, po, aGd, bzD, pm, eF, f, bw, dr, Nq, ck, EW, BU, pc, bzz, iT, azk, bt, pg, oh, bfX, dW, k, pw, dx, QV, bzF); #c1(GATA1) c2(XP_011542199) c3(erythroid transcription factor isoform X1) c4(340682) c5( )c6( )c7(bM, T, aC, xu, bzE, alT, oA, bzA, bzC, oY, ci, hF, aYh, br, ad, er, x, CJ, fX, yw, BR, dE, oW, aT, bzB, h, dz, iY, DU, ae, af, Nr, iU, et, aW, adw, eH, KX, po, aGd, bzD, pm, eF, f, bw, dr, Nq, ck, EW, BU, pc, bzz, iT, azK, bt, pg, oh, bfX, dW, k, pw, dX, QV, bzF); #c1(GATA2) c2(NP_001139133) c3(endothelial transcription factor GATA-2 isoform 1) c4(58732, 58733) c5(58734, 58735, 58736, 58737, 58738, 58739, 58740, 58741, 58742, 58743, 58744) c6( )c7(gV, T, bM, xu, qx, oA, bs, uO, n, jh, j, hF, br, ad, er, dP, av, iu, azD, bzG, tL, aT, Vv, j, an, DY, pk, ae, bzH, yo, s, hG, iU, aW, oV, bvK, gK, CM, lo, gm, akk, cm, sk, bz, bw, ck, ac, eM, iT, dW, PO, PN, oX, bt, KS, B); #c1(GATA2) c2(NP_001139134) c3(endothelial transcription factor GATA-2 isoform 2) c4(58745) c5(58746, 58747, 58748, 58749, 58750, 58751, 58752, 58753, 58754, 58755, 58756, 58757) c6( )c7(gV, T, bM, xu, qx, oA, bs, uO, n, jh, j, hF, br, ad, er, dP, av, iu, azD, bzG, tL, aT, Vv, j, an, DY, pk, ae, bzH, yo, s, hG, iU, aW, oV, bvK, gK, CM, lo, gm, akk, cm, sk, bz, bw, ck, ac, eM, iT, dW, PO, PN, oX, bt, kS, B); #c1(GATA3) c2(NP_002042) c3(trans-acting T-cell-specific transcription factor GATA-3 isoform 2) c4(123560) c5(123561, 123562, 123563, 123564, 123565, 123566, 123567, 123568, 123569, 123570, 123571, 123572, 123573) c6( )c7(bM, T, os, aC, auW, ci, Tz, Ds, K, zl, aW, ml, vs, eE, cm, ou, akl, dW, k, fy, QO, ajm, n, aj, fr, ct, uE, qN, zP, eV, dL, OM, bxg, aVn, oS, A, aiB, sp, OX, JM, at, uz, fl, Dw, eX, aA, cz, bzl, J, di, aT, Cy, ae, aFK, aDm, en, pb, kP, ac, mG, AH, fD, id, jh, oZ, bS, NI, mC, ad, py, MS, oz, ds, HD, lO, ff, cr, eZ, dN, hp, Z, bz, baa, z, dJ, lf, ra, iu, y); #c1 (GATA3) c2(XP_005252500) c3(trans-acting T-cell-specific transcription factor GATA-3 isoform X1) c4(271826, 271827) c5(271828, 271829, 271830, 271831, 271832, 271833) c6( )c7(bM, T, os, aC, auW, ci, Tz, Ds, K, zQ, aW, ml, vs, eE, cm, ou, akl, dW, k, fy, QO, ajm, n, aj, fr, ct, uE, qN, zP, eV, dL, OM, bxg, aVn, oS, A, aiB, sp, OX, JM, at, uz, fl, Dw, eX, aA, cz, bzl, J, di, aT, Cy, ae, aFK, aDm, en, pb, kP, ac, mG, AH, fD, id, jh, oZ, bS, NI, mC, ad, py, MS, oz, ds, hD, lQ, ff, cr, eZ, dN, hp, Z, bz, baa, z, dJ, lf, ra, iu, y); #c1(GATA4) c2(NP_002043) c3(transcription factor GATA-4) c4(123574, 123575) c5(123576, 123577, 123578, 123579, 123580, 123581, 123582, 123583, 123584, 123585, 123586, 123587, 123588, 123589, 123590, 123591, 123592, 123593, 123594) c6( )c7(T, ahf, aC, wl, mr, pf, aAz, aZ, acl, ht, bzO, aw, aA, bB, n, aj, bD, j, ci, BM, q, tO, J, cd, md, eA, av, Mg, mh, aE, cc, nX, BJ, lC, aT, bzJ, bzk, iv, bq, hu, rz, bzl, hm, yi, anR, A, vT, JI, aW, ff, blP, r, aq, aF, Hu, en, bu, cl, t, f, g, as, cm, iO, ac, eM, gY, dB, bE, AM, wk, mx, ll, hL, gZ, Qu, Km, bzM, m, sx, bzN, iu, vQ); #c1(GATA5) c2(XP_006723762) c3(transcription factor GATA-5 isoform X1) c4(295370, 295371) c5(295372, 295373, 295374, 295375, 295376, 295377, 295378, 295379, 295380, 295381, 295382, 295383, 295384, 295385, 295386) c6( )c7(T, aC, pf, acl, ht, aA, bB, n, fr, ci, q, Mg, av, py, aE, aT, hu, Gc, JI, aW, ff, r, af, si, en, g, wU, amF, bE, go, bD, vQ); #c1(GATAG) c2(NP_005248) c3(transcription factor GATA-6) c4(154379) c5(154380, 154381, 154382, 154383, 154384, 154385, 154386) c6( )c7(bL, lD, T, aC, id, mr, aZ, acl, aD, qy, aA, bB, n, bzS, bzR, aj, ci, j, q, J, eA, eJ, dP, bzl, av, jz, mh, aE, Mg, aT, iv, rO, HR, aKG, rz, ly, kl, d, s, vT, aW, Lh, r, bzP, aq, fe, kp, aF, Hu, g, as, LV, t, pb, lx, LB, ali, ac, gY, bE, Qu, wk, ajd, m, bD, lC, KY, bzT, iu); #c1(GATAD1) c2(NP_066990) c3(GATA zinc finger domain-containing protein 1) c4(208065) c5(208066, 208067, 208068, 208069, 208070, 208071) c6( )c7(nJ, bzu, mx); #c1(GATAD2A) c2(NP_001287875) c3(transcriptional repressor p66-alpha isoform 1) c4(113103) c5( )c6( )c7(bL); #c1(GATAD2A) c2(NP_060130) c3(transcriptional repressor p66-alpha isoform 2) c4(194315) c5(194316, 194317, 194318, 194319, 194320, 194321, 194322, 194323, 194324, 194325) c6( ) c7(bL); #c1(GATAD2A) c2(XP_005260013) c3(transcriptional repressor p66-alpha isoform X3) c4(276371, 276372, 276373) c5(276374, 276375, 276376, 276377, 276378, 276379, 276380, 276381, 276382, 276383, 276384) c6( )c7(bL); #c1(GATAD2A) c2(XP_011526406) c3(transcriptional repressor p66-alpha isoform X2) c4(320673, 320674, 320675, 320676) c5(320677, 320678, 320679, 320680, 320681, 320682, 320683, 320684, 320685, 320686, 320687, 320688, 320689, 320690, 320691, 320692, 320693, 320694, 320695, 320696, 320697, 320698, 320699, 320700, 320701, 320702, 320703, 320704, 320705, 320706, 320707, 320708, 320709, 320710, 320711, 320712, 320713) c6( )c7(bL); #c1(GATAD2A) c2(XP_011526409) c3(transcriptional repressor p66-alpha isoform X4) c4(320714) c5( )c6( )c7(bL); #c1(GATAD2A) c2(XP_011526410) c3(transcriptional repressor p66-alpha isoform X5) c4( )c5(320715, 320716, 320717, 320718, 320719, 320720, 320721) c6( )c7(bL); #c1(GATAD2B) c2(XP_006711532) c3(transcriptional repressor p66-beta isoform X1) c4(286062) c5(286063, 286064, 286065, 286066, 286067, 286068) c6( )c7(ae, T, bE, aW, ds, iw, aT, bB, bzV, nA, WI, ik, bz, eA, ad); #c1(GATB) c2(NP_004555) c3 (glutamyl-tRNA(Gln) amidotransferase subunit B, mitochondrial precursor) c4(147615) c5(147616, 147617, 147618, 147619, 147620, 147621) c6( )c7(hb, cz); #c1(GATM) c2(NP_001473) c3(glycine amidinotransferase, mitochondrial precursor) c4(118010, 118011) c5(118012, 118013, 118014, 118015, 118016, 118017, 118018, 118019, 118020, 118021, 118022, 118023) c6( )c7(zl, bzW, E, PQ, ci, dy, bq, j, zP, ae, cd, ad, bc); #c1(GBA) c2(NP_001165282) c3(glucosylceramidase isoform 2) c4( )c5(67551, 67552, 67553, 67554, 67555, 67556, 67557, 67558) c6(67559, 67560) c7(bzZ, T, fM, bzX, jh, fd, GC, xg, aR, bzY, oW, fn, yZ, K, an, af, ak, yr, aGf, dq, aEW, vU, aGe, Ji, U, Lq, lC, aen, sH, ek); #c1(GBA) c2(NP_001165283) c3(glucosylceramidase isoform 3 precursor) c4(67561, 67562, 67563) c5(67564, 67565, 67566, 67567, 67568, 67569, 67570, 67571, 67572, 67573, 67574, 67575, 67576, 67577, 67578, 67579, 67580, 67581, 67582, 67583) c6(67584, 67585) c7(bzZ, T, fM, bzX, jh, fd, GC, xg, aR, bzY, oW, fn, yZ, K, an, af, ak, yr, aGf, dq, aEW, vU, aGe, Ji, U, Lq, lC, aen, sH, ek); #c1(GBA) c2(XP_011507709) c3(glucosylceramidase isoform X1) c4(297739, 297740, 297741, 297742, 297743) c5(297744, 297745, 297746, 297747, 297748, 297749, 297750, 297751, 297752, 297753, 297754, 297755, 297756, 297757, 297758, 297759, 297760, 297761, 297762, 297763, 297764, 297765, 297766, 297767, 297768, 297769, 297770, 297771, 297772, 297773, 297774, 297775, 297776, 297777) c6(297778, 297779, 297780, 297781, 297782, 297783, 297784, 297785, 297786, 297787, 297788) c7(bzZ, T, fM, bzX, jh, fd, GC, xg, aR, bzY, oW, fn, yZ, K, an, af, ak, yr, aGf, dq, aEW, vU, aGe, Ji, U, Lq, lC, aen, sH, ek); #c1(GBA) c2(XP_011545232) c3(glucosylceramidase isoform X1) c4( )c5(344019, 344020, 344021, 344022, 344023, 344024, 344025, 344026, 344027) c6( )c7(bzZ, T, fM, bzX, jh, fd, GC, xg, aR, bzY, oW, fn, yZ, K, an, af, ak, yr, aGf, dq, aEW, vU, aGe, Ji, U, Lq, lC, aen, sH, ek); #c1(GBA2) c2(NP_065995) c3(non-lysosomal glucosylceramidase) c4(206648) c5(206649, 206650, 206651, 206652, 206653, 206654) c6( )c7(vU, ky, bcS, aV, Y, Kk, Lp, Lq, K, bAa); #c1(GBA2) c2(XP_006716872) c3(non-lysosomal glucosylceramidase isoform X2) c4(290532) c5(290533, 290534, 290535, 290536, 290537, 290538) c6( )c7(vU, ky, bcS, aV, Y, Kk, Lp, Lq, K, bAa); #c1(GBA2) c2(XP_011516274) c3(non-lysosomal glucosylceramidase isoform X6) c4( )c5(309482) c6( )c7(vU, ky, bcS, aV, Y, Kk, Lp, Lq, K, bAa); #c1(GBA2) c2(XP_011516275) c3(non-lysosomal glucosylceramidase isoform X7) c4(309483, 309484) c5(309485, 309486, 309487, 309488, 309489, 309490, 309491, 309492, 309493, 309494, 309495) c6( )c7(vU, ky, bcS, aV, Y, Kk, Lp, Lq, K, bAa); #c1(GBAS) c2(NP_001189398) c3(protein NipSnap homolog 2 isoform 2) c4(79358) c5(79359, 79360, 79361, 79362, 79363, 79364) c6( )c7(kC); #c1(GBAS) c2(NP_001474) c3(protein NipSnap homolog 2 isoform 1) c4(118024) c5(118025, 118026, 118027, 118028, 118029, 118030) c6( ) c7(KC); #c1(GBE1) c2(NP_000149) c3(1,4-alpha-glucan-branching enzyme) c4(3377, 3378, 3379, 3380, 3381) c5(3382, 3383, 3384, 3385, 3386, 3387, 3388, 3389, 3390, 3391, 3392, 3393, 3394, 3395, 3396, 3397, 3398, 3399, 3400, 3401, 3402, 3403, 3404, 3405, 3406, 3407, 3408, 3409, 3410, 3411, 3412, 3413, 3414, 3415, 3416, 3417, 3418, 3419, 3420, 3421, 3422, 3423, 3424, 3425, 3426, 3427, 3428, 3429, 3430, 3431, 3432, 3433, 3434, 3435, 3436, 3437, 3438, 3439, 3440, 3441, 3442, 3443, 3444, 3445, 3446, 3447, 3448, 3449, 3450, 3451, 3452, 3453, 3454, 3455, 3456, 3457, 3458, 3459, 3460, 3461, 3462, 3463, 3464, 3465, 3466, 3467, 3468, 3469, 3470, 3471, 3472, 3473, 3474, 3475, 3476, 3477, 3478, 3479, 3480, 3481, 3482, 3483, 3484, 3485, 3486, 3487, 3488, 3489, 3490, 3491, 3492, 3493, 3494, 3495, 3496, 3497, 3498, 3499, 3500, 3501, 3502, 3503, 3504, 3505, 3506, 3507, 3508, 3509, 3510, 3511, 3512, 3513, 3514, 3515, 3516, 3517, 3518, 3519, 3520, 3521, 3522, 3523, 3524, 3525, 3526, 3527, 3528, 3529, 3530, 3531, 3532, 3533, 3534, 3535, 3536, 3537, 3538) c6(3539) c7(bM, gk, cc, aOi, bAd, cb, Bw, hl, Ad, oj, al, bAe, v, ey, bAb, bAc, fq); #c1(GBF1) c2(NP_004184) c3(Golgi-specific brefeldin A-resistance guanine nucleotide exchange factor 1 isoform 1) c4(144163) c5(144164, 144165, 144166, 144167, 144168, 144169) c6( )c7(fR, vi); #c1(GBGT1) c2(NP_001269558) c3(globoside alpha-1,3-N-acetylgalactosaminyltransferase 1 isoform 2) c4(101960) c5(101961, 101962, 101963, 101964, 101965, 101966, 101967, 101968, 101969, 101970) c6( )c7(aA, hp, q, aF, r); #c1(GBGT1) c2(NP_001269561) c3(globoside alpha-1,3-N-acetylgalactosaminyltransferase 1 isoform 3) c4(101986, 101987) c5(101988, 101989, 101990, 101991, 101992, 101993, 101994, 101995, 101996, 101997, 101998) c6( )c7(aA, hp, q, af, r); #c1 (GBGTI) c2(NP_068836) c3(globoside alpha-1,3-N-acetylgalactosaminyltransferase 1 isoform 1) c4(210851, 210852) c5(210853, 210854, 210855, 210856, 210857, 210858, 210859, 210860) c6( )c7(aA, hp, q, aF, r); #c1(GBX1) c2(NP_001092304) c3(homeobox protein GBX-1) c4(39542) c5(39543, 39544, 39545, 39546, 39547, 39548, 39549) c6( )c7(ae, bt, fq, ad); #c1(GBX2) c2(NP_001476) c3(homeobox protein GBX-2 isoform 1) c4(118031) c5(118032, 118033, 118034, 118035, 118036, 118037, 118038) c6( )c7(T, ex, fq, eV, PJ, bt, ae, oc, ad); #c1(GC) c2(NP_000574) c3(vitamin D-binding protein isoform 1 precursor) c4( )c5(12832, 12833, 12834, 12835, 12836) c6( )c7(bL, bM, T, am, hl, eP, eW, al, jh, cz, bB, aj, dG, j, uu, v, pM, fr, V, qW, q, J, ad, ai, dP, kL, eA, av, F, xy, aE, oz, awa, aT, cU, hD, h, dL, afV, K, bq, an, aDp, ae, af, vQ, bA, eu, eL, A, mR, aW, Y, vj, fc, r, yy, aPf, fe, Pd, Z, lk, cM, bN, hq, D, sS, il, Dy, yE, bE, aZL, Be, at, QF, lS, cP, pK, y, B, ao, yv); #c1(GC) c2(NP_001191236) c3(vitamin D-binding protein isoform 3 precursor) c4(81177, 81178, 81179) c5(81180, 81181, 81182, 81183, 81184, 81185, 81186, 81187) c6( ) c7(bL, bM, T, am, hl, eP, eW, al, jh, cz, bB, aj, dG, j, uu, v, pM, fr, V, qW, q, J, ad, ai, dP, kL, eA, av, F, xy, aE, oz, awa, aT, cl, HD, h, dL, afV, K, bq, an, aDp, ae, af, vO, bA, eu, eL, A, mR, aW, Y, vj, fc, r, yy, aPf, fe, Pd, Z, lk, cM, bN, hq, D, sS, il, Dy, yE, bE, aZL, Be, at, QF, lS, cP, pK, y, B, ao, yv); #c1(GC) c2(XP_006714240) c3(vitamin D-binding protein isoform X1) c4( )c5(287659, 287660, 287661, 287662, 287663) c6( ) c7(bL, bM, T, am, hl, eP, eW, al, jh, cz, bB, aj, dG, j, uu, v, pM, fr, V, qW, q, J, ad, ai, dP, kL, eA, av, F, xy, aE, oz, awa, aT, cU, hD, h, dL, afV, K, bq, an, aDp, ae, af, vO, bA, eu, eL, A, mR, aW, Y, vj, fc, r, yy, aPf, fe, Pd, Z, lk, cM, bN, hq, D, sS, il, Dy, yE, bE, aZL, Be, at, QF, lS, cP, pK, y, B, ao, yv); #c1(GCA) c2(NP_036330) c3(grancalcin) c4(173033) c5(173034, 173035, 173036, 173037, 173038, 173039) c6( )c7(bM, T, l, eM, kq, r, cm, it, aiC, v, j, K, app, pw, he, V, Pr, q, WW, bAf); #c1(GCC1) c2(NP_078799) c3(GRIP and coiled-coil domain-containing protein 1) c4(215527) c5(215528, 215529, 215530, 215531, 215532, 215533) c6( )c7(jb, cu, acl); #c1(GCDH) c2(NP_000150) c3(glutaryl-CoA dehydrogenase, mitochondrial isoform a precursor) c4(3540, 3541, 3542) c5(3543, 3544, 3545, 3546, 3547, 3548, 3549, 3550, 3551, 3552, 3553, 3554) c6( )c7(mO, xT, awC, aU, n, dq, K, rL, dr); #c1(GCDH) c2(NP_039663) c3(glutaryl-CoA dehydrogenase, mitochondrial isoform b precursor) c4(177203) c5(177204, 177205, 177206, 177207, 177208, 177209, 177210, 177211, 177212, 177213, 177214, 177215) c6( ) c7(mO, xT, awC, aU, n, dq, K, rL, dr); #c1(GCFC2) c2(NP_003194) c3(GC-rich sequence DNA-binding factor 2 isoform 1) c4(136164, 136165, 136166) c5(136167, 136168, 136169, 136170, 136171, 136172, 136173, 136174, 136175, 136/76, 136177, 136178) c6( )c7(ajl, ci, yA, d, k); #c1 (GCFC2) c2(XP_005264577) c3(GC-rich sequence DNA-binding factor 2 isoform X1) c4( )c5(279108, 279109) c6( )c7(ajl, ci, yA, d, k); #c1 (GCFC2) c2(XP_011531376) c3(GC-rich sequence DNA-binding factor 2 isoform X2) c4( )c5(327377) c6( )c7(ajl, ci, yA, d, k); #c1(GCFC2) c2(XP_011531377) c3(GC-rich sequence DNA-binding factor 2 isoform X3) c4( )c5(327378, 327379, 327380, 327381, 327382) c6( )c7(ajl, ci, yA, d, k); #c1(GCG) c2(NP_002045) c3(glucagon preproprotein) c4(123595, 123596, 123597, 123598, 123599, 123600, 123601) c5(123602, 123603, 123604, 123605, 123606, 123607, 123608, 123609, 123610, 123611, 123612, 123613, 123614, 123615, 123616, 123617, 123618, 123619, 123620, 123621, 123622, 123623, 123624, 123625, 123626, 123627, 123628, 123629, 123630, 123631, 123632, 123633, 123634, 123635, 123636, 123637, 123638, 123639, 123640, 123641, 123642, 123643, 123644, 123645, 123646, 123647, 123648, 123649, 123650, 123651, 123652, 123653, 123654, 123655, 123656, 123657, 123658, 123659, 123660, 123661, 123662, 123663, 123664, 123665, 123666, 123667, 123668, 123669, 123670, 123671, 123672, 123673, 123674, 123675, 123676, 123677, 123678, 123679, 123680, 123681, 123682, 123683, 123684, 123685, 123686, 123687, 123688, 123689, 123690, 123691, 123692, 123693, 123694, 123695, 123696, 123697, 123698, 123699, 123700, 123701, 123702, 123703, 123704, 123705, 123706, 123707, 123708, 123709, 123710, 123711, 123712, 123713, 123714, 123715, 123716, 123717, 123718, 123719, 123720, 123721, 123722, 123723, 123724, 123725, 123726, 123727, 123728, 123729, 123730, 123731, 123732, 123733, 123734, 123735, 123736, 123737, 123738, 123739, 123740, 123741, 123742, 123743, 123744, 123745, 123746, 123747, 123748, 123749, 123750, 123751, 123752, 123753, 123754, 123755, 123756, 123757, 123758, 123759, 123760, 123761, 123762, 123763, 123764, 123765, 123766, 123767, 123768, 123769, 123770, 123771, 123772, 123773, 123774, 123775, 123776, 123777, 123778, 123779, 123780, 123781, 123782, 123783, 123784, 123785, 123786, 123787, 123788, 123789, 123790, 123791, 123792, 123793, 123794, 123795, 123796, 123797, 123798, 123799, 123800, 123801, 123802, 123803, 123804, 123805, 123806, 123807, 123808, 123809, 123810, 123811, 123812, 123813, 123814, 123815, 123816, 123817, 123818, 123819, 123820, 123821, 123822, 123823, 123824, 123825, 123826, 123827, 123828, 123829, 123830, 123831, 123832, 123833, 123834, 123835, 123836, 123837, 123838, 123839, 123840, 123841, 123842, 123843, 123844, 123845, 123846, 123847, 123848, 123849, 123850, 123851, 123852, 123853, 123854, 123855, 123856, 123857, 123858, 123859, 123860, 123861, 123862, 123863, 123864, 123865, 123866, 123867, 123868, 123869, 123870, 123871, 123872, 123873, 123874, 123875, 123876, 123877, 123878, 123879, 123880, 123881, 123882, 123883, 123884, 123885, 123886, 123887, 123888, 123889, 123890, 123891, 123892, 123893, 123894, 123895, 123896, 123897, 123898, 123899, 123900, 123901, 123902, 123903, 123904, 123905, 123906, 123907, 123908, 123909, 123910, 123911, 123912, 123913, 123914, 123915, 123916, 123917, 123918, 123919, 123920, 123921, 123922, 123923, 123924, 123925, 123926, 123927, 123928, 123929, 123930, 123931, 123932, 123933, 123934, 123935, 123936, 123937, 123938, 123939, 123940, 123941, 123942, 123943, 123944, 123945, 123946, 123947, 123948, 123949, 123950, 123951, 123952, 123953, 123954, 123955, 123956, 123957, 123958, 123959, 123960, 123961, 123962, 123963, 123964, 123965, 123966, 123967, 123968, 123969, 123970, 123971, 123972, 123973, 123974, 123975, 123976, 123977, 123978, 123979, 123980, 123981, 123982, 123983, 123984, 123985, 123986, 123987, 123988, 123989, 123990, 123991, 123992, 123993, 123994, 123995, 123996, 123997, 123998, 123999, 124000, 124001, 124002, 124003, 124004, 124005, 124006, 124007, 124008, 124009, 124010, 124011, 124012, 124013, 124014, 124015, 124016, 124017, 124018, 124019, 124020, 124021, 124022, 124023, 124024, 124025, 124026, 124027, 124028, 124029, 124030, 124031, 124032, 124033, 124034, 124035, 124036, 124037, 124038, 124039, 124040, 124041, 124042, 124043, 124044, 124045, 124046, 124047, 124048, 124049, 124050, 124051, 124052) c6(124053, 124054, 124055, 124056, 124057) c7(bL, T, dD, dA, pY, ea, e, al, bAg, aw, cz, bB, c, dG, j, aj, v, eq, nS, cd, J, ad, ip, mj, aE, iX, pX, bAi, ju, aT, kf, vH, blg, bci, jy, K, ji, da, fb, XI, ae, aPB, hm, De, eG, A, qZ, De, aW, baT, QM, hl, ap, cr, yc, dq, kp, app, g, Sm, cm, KM, bAh, iO, gk, bE, bAj, bo, Ji, mx, U, fP, gZ, QF, mf, aS, aJQ, ei, ra, X, VB); #c1(GCGR) c2(XP_006722340) c3(glucagon receptor isoform X1) c4(294682) c5(294683, 294684, 294685, 294686, 294687, 294688) c6( )c7(bL, qa, mj, eR, pX, cz, ahJ, blg, mf, dG, aj, v, J, bJ); #c1(GCH1) c2(NP_001019195) c3(GTP cyclohydrolase 1 isoform 1) c4(27601) c5(27602, 27603, 27604, 27605, 27606, 27607, 27608, 27609, 27610, 27611, 27612, 27613, 27614, 27615, 27616, 27617, 27618, 27619, 27620, 27621, 27622, 27623, 27624, 27625, 27626, 27627, 27628, 27629, 27630, 27631, 27632, 27633, 27634, 27635, 27636, 27637, 27638, 27639, 27640, 27641, 27642, 27643, 27644, 27645, 27646, 27647, 27648, 27649, 27650, 27651, 27652, 27653, 27654, 27655, 27656, 27657, 27658, 27659, 27660, 27661, 27662, 27663, 27664, 27665, 27666, 27667, 27668, 27669, 27670, 27671, 27672, 27673, 27674, 27675, 27676, 27677, 27678, 27679, 27680, 27681, 27682, 27683, 27684, 27685, 27686, 27687, 27688, 27689, 27690, 27691, 27692, 27693, 27694, 27695, 27696, 27697, 27698, 27699, 27700, 27701, 27702) c6(27703, 27704, 27705, 27706, 27707) c7(T, bfb, dD, qX, Pt, qb, ea, yk, e, yY, bAp, bO, cz, aNN, cR, OR, dG, j, Sv, uu, aj, eq, acU, aWT, bAo, ai, J, bAt, aR, ahR, HF, bgk, aji, DK, hb, M, Wk, aU, h, bK, Wm, bAm, bAr, an, bAl, Gc, aTm, Wi, yL, dN, HL, gu, cm, xw, dr, bN, cv, bAn, bAs, yf, cA, HP, mx, VU, uJ, aS, bAq, aks, aCS, bAk, aWX, WI, y, afX, rf); #c1(GCH1) c2(NP_001019242) c3(GTP cyclohydrolase 1 isoform 3) c4(27708) c5(27709, 27710, 27711, 27712, 27713, 27714, 27715, 27716) c6(27717, 27718) c7(T, bfb, dD, qX, Pt, qb, ea, yk, e, yY, bAp, bO, cz, aNN, cR, oR, dG, j, Sv, uu, aj, eq, acU, aWT, bAo, ai, J, bAt, aR, ahR, HF, bgk, aji, DK, hb, M, Wk, aU, h, bK, Wm, bAm, bAr, an, bAl, Gc, aTm, Wi, yL, dN, HL, gu, cm, xw, dr, bN, cv, bAn, bAs, yf, cA, HP, mx, VU, uJ, aS, bAq, aks, aCS, bAk, aWX, WI, y, afX, rf); #c1(GCH1) c2(XP_005267587) c3(GTP cyclohydrolase 1 isoform X1) c4(281033, 281034) c5(281035, 281036, 281037, 281038, 281039, 281040, 281041, 281042, 281043, 281044, 281045, 281046, 281047, 281048, 281049, 281050) c6(281051, 281052, 281053, 281054) c7(T, bfb, dD, qX, Pt, qb, ea, yk, e, yY, bAp, bO, cz, aNN, cR, OR, dG, j, Sv, uu, aj, eq, acU, aWT, bAo, ai, J, bAt, aR, ahR, HF, bgk, aji, DK, hb, M, Wk, aU, h, bK, Wm, bAm, bAr, an, bAl, Gc, aTm, Wi, yL, dN, HL, gu, cm, xw, dr, bN, cv, bAn, bAs, yf, cA, HP, mx, VU, uJ, aS, bAq, aks, aCS, bAk, aWX, Wl, y, afX, rf); #c1(GCHFR) c2(NP_005249) c3(GTP cyclohydrolase 1 feedback regulatory protein) c4(154387) c5(154388, 154389, 154390, 154391, 154392, 154393) c6( )c7(hl, acu, aR, hX, hb, dV, aU, af, A, Y, eH, Pg, eZ, as, eF, cm, bN, kT, WO, ac, WP, U, xg, ek); #c1(GCK) c2(NP_000153) c3(glucokinase isoform 1) c4(3555) c5(3556, 3557, 3558, 3559, 3560, 3561) c6( )c7(bL, aC, jv, dD, eR, yG, bAu, bAg, cz, cl, dG, j, fu, aj, v, eq, GS, add, J, md, aPW, mj, mh, xE, aE, bza, ep, jy, K, bq, af, yo, A, wr, lR, bAv, fc, eZ, fQ, dq, aq, EZ, bAw, mb, dr, qa, mi, akm, bo, mg, mf, dy, cP, ei, y, B, BA, yv); #c1(GCK) c2(NP_277042) c3(glucokinase isoform 2) c4(228793) c5(228794, 228795, 228796, 228797, 228798, 228799) c6( )c7(bL, aC, jv, dD, eR, yG, bAu, bAg, cz, cQ, dG, j, fu, aj, v, eq, GS, add, J, md, aPW, mj, mh, xE, aE, bza, ep, jy, K, bq, af, yo, A, wr, lR, bAv, fc, eZ, fQ, dq, aq, EZ, bAw, mb, dr, qa, mi, akm, bo, mg, mf, dy, cP, ei, y, B, BA, yv); #c1(GCK) c2(NP_277043) c3(glucokinase isoform 3) c4(228800) c5(228801, 228802, 228803, 228804, 228805, 228806) c6( ) c7(bL, aC, jv, dD, eR, yG, bAu, bAg, cz, cQ, dG, j, fu, aj, v, eq, GS, add, J, md, aPW, mj, mh, xE, aE, bza, ep, jy, K, bq, af, yo, A, wr, lR, bAv, fc, eZ, fQ, dq, aq, EZ, bAw, mb, dr, qa, mi, akm, bo, mg, mf, dy, cP, ei, y, B, BA, yv); #c1(GCKR) c2(NP_001477) c3(glucokinase regulatory protein) c4(118039, 118040) c5(118041, 118042, 118043, 118044, 118045, 118046, 118047) c6( )c7(bL, nD, dD, eR, e, aw, bAg, qQ, aj, dG, j, v, eq, aY, J, mj, aE, M, h, sj, nn, fc, fe, iV, ac, c0, mg, dy, cP, ei); #c1(GCKR) c2(XP_011531064) c3 (glucokinase regulatory protein isoform X2) c4( )c5(327030, 327031, 327032, 327033, 327034) c6( )c7(bL, nD, dD, eR, e, aw, bAg, qQ, aj, dG, j, v, eq, aY, J, mj, aE, M, h, sj, nn, fc, fe, iV, ac, c0, mg, dy, cP, ei); #c1(GCLC) c2(NP_001489) c3(glutamate-cysteine ligase catalytic subunit isoform a) c4(118157) c5(118158, 118159, 118160, 118161, 118162, 118163) c6( )c7(bM, T, dD, eR, e, aw, cz, bB, fd, arl, gA, j, fu, qf, eq, q, ad, av, ft, ib, aE, bAy, aT, ab, K, he, ae, rl, aa, A, d, B, anv, nO, aW, bAx, r, aq, S, g, as, t, cm, bz, UZ, Hp, ck, ac, eM, bE, uy, bo, at, bzm, m, fD, y, bt, X, ao); #c1(GCM1) c2(NP_003634) c3(chorion-specific transcription factor GCMa) c4(139760) c5(139761, 139762, 139763, 139764, 139765, 139766, 139767) c6( )c7(nJ, HI, jv, e, cz, eb, lo, ey, uC, af); #c1(GCM2) c2(XP_011513293) c3(chorion-specific transcription factor GCMb isoform X1) c4(305579) c5(305580, 305581, 305582, 305583, 305584, 305585) c6( )c7(HI, T, s, OX, vj, Lm, wo, iS, azg, bAz); #c1(GCN1L1) c2(NP_006827) c3(translational activator GCN1) c4(168482) c5(168483, 168484, 168485, 168486, 168487, 168488) c6( )c7(jf, pi, fr); #c1(GCNT1) c2(XP_0067/7115) c3(beta-1,3-galactosyl-O-glycosyl-glycoprotein beta-1,6-N-acetylglucosaminyltransferase isoform X1) c4(290734) c5(290735, 290736, 290737, 290738, 290739, 290740, 290741, 290742, 290743, 290744, 290745, 290746, 290747, 290748, 290749, 290750, 290751, 290752) c6( )c7(ae, aC, B, aE, bE, oe, r, cz, n, Ld, ci, g, bq, bz, q, ad); #c1(GCNT2) c2(NP_001482) c3(N-acetyllactosaminide beta-1,6-N-acetylglucosaminyl-transferase isoform B) c4(118056) c5(118057, 118058, 118059, 118060, 118061, 118062) c6( )c7(ID, bM, T, xS, ea, zs, bAH, e, if, Av, fd, bB, ame, qj, ci, bh, aGX, mF, NF, OB, AW, xy, vV, aE, il, M, bAJ, dV, BZ, K, hu, eL, bc, aiJ, ly, yo, Jl, aW, WE, az, asD, nY, UJ, hN, YG, g, as, eE, eF, cm, bw, iP, aUF, eM, mx, dW, wx, bAD, m, bpy, nD, btc, dD, gU, vl, bAB, aw, ht, aFc, ez, n, amk, aj, fr, nA, uE, qN, Za, qW, ai, br, eW, KL, Vs, bAK, ft, pe, wG, ql, Bj, h, eV, bxi, dl, ey, hB, SD, on, dx, A, mR, bAG, bAF, aRL, fe, aq, kp, mo, boD, Ld, lx, jq, bAL, Qy, bP, dB, w, Qu, aZL, ln, FV, bm, hR, pg, Xa, anx, it, B, bL, fM, dA, KA, bkY, hx, jx, eX, aA, cz, zN, hz, j, bAM, wy, V, q, J, o, eA, cF, DK, hb, aWc, BJ, bza, ld, Vr, aT, Tj, bau, ae, jf, bAE, hM, kl, PK, gP, s, iU, Y, r, lo, bAA, en, t, nC, iK, pb, dS, ck, cT, sS, iM, il, hA, ig, mG, dh, gX, hO, wR, dy, im, zu, bt, aps, ars, ao, aaT, ha, Me, jh, uu, jE, fu, eq, hF, bAl, aGW, ad, BY, qC, hX, av, bAC, F, bnC, wD, vJ, ds, alg, qh, oW, io, hD, lQ, vO, Yi, uQ, d, je, BO, azX, atA, yx, aFE, ff, eZ, CF, Ej, aF, xH, hp, EZ, iV, bz, dr, LB, bF, DT, eU, pc, bE, Lj, z, aCI, dX, aS, XO, bC, od, be, y, WX, iu, hv, pC); #c1(GCNT2) c2(NP_663630) c3(N-acetyllactosaminide beta-1,6-N-acetylglucosaminyl-transferase isoform C) c4(241375) c5(241376, 241377, 241378, 241379, 241380, 241381) c6( )c7(ID, bM, T, xS, ea, zs, bAH, e, if, Av, fd, bB, ame, qj, ci, bh, aGX, mF, NF, OB, AW, xy, vV, aE, il, M, bAJ, dV, BZ, K, hu, eL, bc, aiJ, ly, yo, JI, aW, WE, az, asD, nY, UJ, hN, YG, g, as, BE, eF, cm, bw, iP, aUF, eM, mx, dW, wx, bAD, m, bpy, nD, btc, dD, gU, vl, bAB, aw, ht, aFc, ez, n, amk, aj, fr, nA, uE, qN, Za, qW, ai, br, eW, KL, Vs, bAK, ft, pe, wG, ql, Bj, h, eV, bxi, dL, ey, hB, SD, on, dx, A, mR, bAG, bAF, aRL, fe, aq, kp, mo, boD, Ld, lx, jq, bAL, Qy, bP, dB, w, Qu, aZL, ln, FV, bm, hR, pg, Xa, anx, it, B, bL, fM, dA, kA, bkY, hx, jx, eX, aA, cz, zN, hz, j, bAM, wy, V, q, J, o, eA, cF, DK, hb, aWc, BJ, bza, ld, Vr, aT, Tj, bau, ae, jf, bAE, hM, kl, PK, gP, s, iU, Y, r, lo, bAA, en, t, nC, ik, pb, dS, ck, cT, sS, iM, il, hA, ig, mG, dh, gX, hO, wR, dy, im, zu, bt, aps, ars, ao, aaT, ha, Me, jh, uu, jE, fu, eq, hF, bAl, aGW, ad, BY, qC, hX, av, bAC, F, bnC, wD, vJ, ds, alg, qh, oW, io, HD, lQ, vO, Yi, uQ, d, je, BO, azX, atA, yx, aFE, ff, eZ, CF, Ej, aF, xH, hp, EZ, iV, bz, dr, LB, bF, DT, eU, pc, bE, Lj, z, aCl, dX, aS, XO, bC, oQ, be, y, WX, iu, hv, pC); #c1(GCNT2) c2(XP_006715115) c3(N-acetyllactosaminide beta-1,6-N-acetylglucosaminyl-transferase isoform X1) c4(288734) c5(288735, 288736, 288737, 288738, 288739, 288740, 288741, 288742, 288743, 288744) c6( )c7(ID, bM, T, xS, ea, zs, bAH, e, if, Av, fd, bB, ame, qj, ci, bh, aGX, mF, NF, oB, AW, xy, vV, aE, il, M, bAJ, dV, BZ, K, hu, eL, bc, aiJ, ly, yo, JI, aW, WE, az, asD, nY, UJ, hN, YG, g, as, BE, eF, cm, bw, iP, aUF, eM, mx, dW, wx, bAD, m, bpy, nD, btc, dD, gU, vl, bAB, aw, ht, aFc, ez, n, amk, aj, fr, nA, uE, qN, Za, qW, ai, br, eW, KL, Vs, bAK, ft, pe, wG, ql, Bj, h, eV, bxi, dl, ey, HB, SD, on, dx, A, mR, bAG, bAF, aRL, fe, aq, kp, mo, boD, Ld, lx, jq, bAL, Qy, bP, dB, w, Qu, aZL, ln, FV, bm, hR, pg, Xa, anx, it, B, bL, fM, dA, KA, bkY, hx, jx, eX, aA, cz, zN, hz, j, bAM, wy, V, q, J, o, eA, cF, DK, hb, aWc, BJ, bza, ld, Vr, aT, Tj, bau, ae, jf, bAE, hM, kl, PK, gP, s, il, Y, r, lo, bAA, en, t, nC, ik, pb, dS, ck, cT, sS, iM, il, hA, ig, mG, dh, gX, hO, wR, dy, im, zu, bt, aps, ars, ao, aaT, ha, Me, jh, uu, jE, fu, eq, hF, bAl, aGW, ad, BY, qC, hX, av, bAC, F, bnC, wD, vJ, ds, alg, qh, oW, io, hD, lQ, vO, Yi, uQ, d, je, BO, azX, atA, yx, aFE, ff, eZ, CF, Ej, aF, xH, hp, EZ, iV, bz, dr, LB, bF, DT, eU, pc, bE, Lj, z, aCl, dX, aS, XO, bC, oQ, be, y, WX, iu, hv, pC); #c1(GCNT2) c2(XP_011512768) c3 (N-acetyllactosaminide beta-1,6-N-acetylglucosaminyl-transferase isoform X5) c4(303846, 303847) c5(303848, 303849, 303850, 303851, 303852, 303853) c6( ) c7(ID, bM, T, xS, ea, zs, bAH, e, if, Av, fd, bB, ame, qj, ci, bh, aGX, mF, NF, OB, AW, xy, vV, aE, il, M, bAJ, dV, BZ, K, hu, eL, bc, aiJ, ly, yo, JI, aW, WE, az, asD, nY, UJ, HN, YG, g, as, eE, eF, cm, bw, iP, aUF, eM, mx, dW, wx, bAD, m, bpy, nD, btc, dD, gU, vl, bAB, aw, ht, afc, ez, n, amk, aj, fr, nA, uE, qN, Za, qW, ai, br, eW, KL, Vs, bAK, ft, pe, wG, ql, Bj, h, eV, bxi, dL, ey, hB, SD, on, dx, A, mR, bAG, bAF, aRL, fe, aq, kp, mo, boD, Ld, lx, jq, bAL, Qy, bP, dB, w, Qu, aZL, ln, FV, bm, hR, pg, Xa, anx, it, B, bL, fM, dA, KA, bkY, hx, jx, eX, aA, cz, zN, hz, j, bAM, wy, V, q, J, o, eA, cF, DK, hb, aWc, BJ, bza, ld, Vr, aT, Tj, bau, ae, jf, bAE, hM, kl, PK, gP, s, il, Y, r, lo, bAA, en, t, nC, ik, pb, dS, ck, cT, sS, iM, il, hA, ig, mG, dh, gX, hO, wR, dy, im, zu, bt, aps, ars, ao, aaT, ha, Me, jh, uu, jE, fu, eq, hF, bAl, aGW, ad, BY, qC, hX, av, bAC, F, bnC, wD, vJ, ds, alg, qh, oW, io, hD, lQ, vO, Yi, uQ, d, je, BO, azX, atA, yx, aFE, ff, eZ, CF, Ej, aF, xH, hp, EZ, iV, bz, dr, LB, bF, DT, eU, pc, bE, Lj, z, aCl, dX, aS, XO, bC, oQ, be, y, WX, iu, hv, pC); #c1(GCNT2) c2(XP_011512770) c3(N-acetyllactosaminide beta-1,6-N-acetylglucosaminyl-transferase isoform X7) c4( ) c5(303854, 303855, 303856, 303857, 303858, 303859) c6( )c7(ID, bM, T, xS, ea, zs, bAH, e, if, Av, fd, bB, ame, qj, ci, bh, aGX, mF, NF, oB, AW, xy, vV, aE, il, M, bAJ, dV, BZ, K, hu, eL, bc, aiJ, ly, yo, JI, aW, WE, az, asD, nY, UJ, hN, YG, g, as, eE, eF, cm, bw, iP, aUF, eM, mx, dW, wx, bAD, m, bpy, nD, btc, dD, gU, vl, bAB, aw, ht, aFc, ez, n, amk, aj, fr, nA, uE, qN, Za, qW, ai, br, eW, KL, Vs, bAK, ft, pe, wG, ql, Bj, h, eV, bxi, dL, ey, hB, SD, on, dx, A, mR, bAG, bAF, aRL, fe, aq, kp, mo, boD, Ld, lx, jq, bAL, Qy, bP, dB, w, Qu, aZL, ln, FV, bm, hR, pg, Xa, anx, it, B, bL, fM, dA, KA, bkY, hx, jx, eX, aA, cz, zN, hz, j, bAM, wy, V, q, J, o, eA, cF, DK, hb, aWc, BJ, bza, ld, Vr, aT, Tj, bau, ae, jf, bAE, hM, kl, PK, gP, s, iU, Y, r, lo, bAA, en, t, nC, iK, pb, dS, ck, cT, sS, iM, il, hA, ig, mG, dh, gX, hO, wR, dy, im, zu, bt, aps, ars, ao, aaT, ha, Me, jh, uu, jE, fu, eq, hF, bAl, aGW, ad, BY, qC, hX, av, bAC, F, bnC, wD, vJ, ds, alg, qh, oW, io, HD, lQ, vO, Yi, uQ, d, je, BO, azX, atA, yx, aFE, ff, eZ, CF, Ej, aF, xH, hp, EZ, iV, bz, dr, LB, bF, DT, eU, pc, bE, Lj, z, aCl, dX, aS, XO, bC, oQ, be, y, WX, iu, hv, pC); #c1(GCNT3) c2(NP_004742) c3(beta-1,3-galactosyl-O-glycosyl-glycoprotein beta-1,6-N-acetylglucosaminyltransferase 3) c4(149193, 149194, 149195, 149196) c5(149197, 149198, 149199, 149200, 149201, 149202, 149203, 149204, 149205, 149206, 149207, 149208, 149209, 149210, 149211, 149212, 149213, 149214, 149215, 149216, 149217, 149218, 149219, 149220, 149221, 149222, 149223, 149224) c6( )c7(T, aE, bE, r, bB, q, X); #c1(GCNT7) c2(NP_542182) c3(beta-1,3-galactosyl-O-glycosyl-glycoprotein beta-1,6-N-acetylglucosaminyltransferase 7) c4(232248) c5(232249, 232250, 232251, 232252, 232253, 232254) c6( )c7(q, aE, r); #c1(GCOM1) c2(NP_001018100) c3(GRINL1A combined protein isoform 1) c4(27327) c5(27328, 27329, 27330, 27331, 27332, 27333) c6( )c7(ae, j, K, ad); #c1(GCOM1) c2(NP_001018101) c3(GRINL1A combined protein isoform 2) c4(27334) c5(27335, 27336, 27337, 27338, 27339, 27340) c6( )c7(ae, j, K, ad); #c1(GCSAM) c2(NP_001177188) c3(germinal center-associated signaling and motility protein isoform 3) c4(72501) c5(72502, 72503, 72504, 72505, 72506, 72507) c6( )c7(fM, ex, bO, jh, fr, eW, eA, cU, eV, bK, eG, Uf, d, ff, en, cA, bE, eD, hO, Lq, R, B, hS); #c1(GCSAM) c2(NP_001177189) c3(germinal center-associated signaling and motility protein isoform 4) c4(72508) c5(72509, 72510, 72511, 72512, 72513, 72514) c6( )c7(fM, ex, bO, jh, fr, eW, eA, cU, eV, bK, eG, Uf, d, ff, en, cA, bE, eD, hO, Lq, R, B, hS); #c1(GCSAM) c2(NP_689998) c3(germinal center-associated signaling and motility protein isoform 1) c4(244681) c5(244682, 244683, 244684, 244685, 244686, 244687) c6( )c7(fM, ex, bO, jh, fr, eW, eA, cU, eV, bK, eG, Uf, d, ff, en, cA, bE, eD, hO, Lq, R, B, hS); #c1(GCSAM) c2(XP_005247329) c3(germinal center-associated signaling and motility protein isoform X4) c4( ) c5(267798) c6( )c7(fM, eX, bO, jh, fr, eW, eA, cU, eV, bK, eG, Uf, d, ff, en, cA, bE, eD, hO, Lq, R, B, hS); #c1 (GCSAML) c2(NP_001268764) c3(germinal center-associated signaling and motility-like protein isoform b) c4(100223) c5(100224, 100225, 100226, 100227, 100228, 100229, 100230, 100231, 100232, 100233, 100234, 100235, 100236) c6( )c7(Ea); #c1(GCSAML) c2(NP_660321) c3(germinal center-associated signaling and motility-like protein isoform a) c4(240899, 240900) c5(240901, 240902, 240903, 240904, 240905, 240906, 240907, 240908, 240909, 240910, 240911, 240912) c6( )c7(Ea); #c1(GCSAML) c2(XP_011542402) c3(germinal center-associated signaling and motility-like protein isoform X3) c4(340940) c5(340941, 340942, 340943, 340944, 340945, 340946, 340947) c6( ) c7(Ea); #c1(GCSH) c2(NP_004474) c3(glycine cleavage system H protein, mitochondrial precursor) c4(146689) c5(146690, 146691, 146692, 146693, 146694, 146695) c6( )c7(y, bAN, UZ, jh); #c1(GDA) c2(NP_001229434) c3(guanine deaminase isoform a) c4(84421, 84422) c5(84423, 84424, 84425, 84426, 84427, 84428, 84429, 84430, 84431, 84432, 84433, 84434) c6( )c7(bAO, zP, zQ, ci, c); #c1(GDA) c2(NP_001229436) c3(guanine deaminase isoform c) c4(84435, 84436) c5(84437, 84438, 84439, 84440, 84441) c6( ) c7(bAD, zP, zQ, ci, c); #c1 (GDA) c2(NP_004284) c3(guanine deaminase isoform b) c4(144835) c5(144836, 144837, 144838, 144839, 144840, 144841, 144842, 144843, 144844, 144845, 144846) c6( )c7(bAD, zP, zQ, ci, c); #c1(GDA) c2(XP_011517520) c3(guanine deaminase isoform X5) c4(311307) c5( )c6( )c7(bAO, zP, zQ, ci, c); #c1(GDA) c2(XP_011517522) c3(guanine deaminase isoform X7) c4(311308) c5(311309, 311310, 311311, 311312, 311313, 311314) c6( )c7(bAO, zP, zQ, ci, c); #c1(GDA) c2(XP_011517525) c3(guanine deaminase isoform X9) c4( )c5(311315, 311316, 311317, 311318, 311319, 311320, 311321, 311322, 311323, 311324, 311325, 311326) c6( )c7(bAO, zP, zQ, ci, c); #c1(GDAP1) c2(NP_001035808) c3(ganglioside-induced differentiation-associated protein 1 isoform b) c4( )c5(33137, 33138, 33139, 33140, 33141) c6( )c7(bAR, Rz, ob, Pt, aw, Jz, fu, bX, K, bu, bAP, bT, bhi, bAS, cm, kC, ql, U, bxk, bAR, y, aof); #c1(GDAP1) c2(NP_061845) c3 (ganglioside-induced differentiation-associated protein 1 isoform a) c4(200745, 200746) c5(200747, 200748, 200749, 200750, 200751, 200752, 200753) c6( )c7(bAl, Rz, ob, Pt, aw, Jz, fu, bX, K, bU, bAP, bT, bhi, bAS, cm, kC, ql, U, bxk, bAR, y, aof); #c1(GDE1) c2(NP_057725) c3(glycerophosphodiester phosphodiesterase 1) c4(192526) c5(192527, 192528, 192529, 192530, 192531, 192532) c6( )c7(oO, T, jv, xo, aA, fd, n, axk, Gn, C, eA, br, ad, aoH, fx, py, aT, xp, ae, yo, mR, nO, aW, oV, ff, eH, r, aq, aF, en, g, eF, t, iV, pm, bw, ck, eM, cj, hR, m, rA, B); #c1(GDF1) c2(NP_001483) c3(embryonic growth/differentiation factor 1 precursor) c4(118063) c5(118064, 118065, 118066, 118067, (18068, 118069) c6( )c7(md, ali, sS, hd, BT, nB, bAT, aW, Lf, bwE, aT, cj, aln, bu, bD, bz, zn); #c1(GDF10) c2(NP_004953) c3(bone morphogenetic protein 38 precursor) c4(151426, 151427) c5(151428, 151429, 151430, 151431, 151432, 151433, 151434, 151435) c6(151436, 151437) c7(qJ, av, T, eG, ib, x, eM, qt, aw, eH, NT, aT, j, K, eF, bz); #c1(GDF10) c2(XP_006717825) c3(bone morphogenetic protein 3B isoform X1) c4( )c5(291369, 291370, 291371, 291372, 291373, 291374, 291375, 291376) c6(291377, 291378) c7(qJ, av, T, eG, ib, x, eM, qt, aw, eH, NT, aT, j, K, eF, bz); #c1(GDF11) c2(XP_006719257) c3(growth/differentiation factor 1l isoform X1) c4(292356, 292357) c5(292358, 292359, 292360, 292361, 292362, 292363, 292364, 292365, 292366, 292367, 292368, 292369) c6( )c7(Lk, T, q, L1, r); #c1(GDF15) c2(NP_004855) c3(growth/differentiation factor 15 precursor) c4(150324) c5(150325, 150326, 150327, 150328, 150329, 150330, 150331, 150332, 150333, 150334) c6( )c7(ID, T, aC, aiR, ea, if, e, bB, ci, aE, K, eL, avn, cp, Qe, hm, ly, aW, pl, aVc, eM, nB, wx, m, pw, P, dD, aw, n, aj, qN, cd, Yc, dL, KV, aq, lx, dB, kz, at, B, bL, KA, jx, aA, cR, cz, v, j, q, J, eA, aT, cy, ae, af, s, Y, r, en, t, ik, BU, ac, gX, LS, awY, yv, gx, Fb, eq, Fc, ad, BY, av, hd, sO, NM, d, ff, yy, aF, bz, qa, pu, bE, y, iu); #c1(GDF2) c2(NP_057288) c3(growth/differentiation factor 2 preproprotein) c4(189877) c5(189878, 189879, 189880, 189881, 189882, 189883) c6( )c7(Cj, aE, aW, oe, al, bAU, eH, aT, cy, he, m, eF, t, Cb, ae, B, ad, wC); #c1(GDF3) c2(NP_065685) c3 (growth/differentiation factor 3 precursor) c4(204829) c5(204830, 204831, 204832, 204833, 204834, 204835) c6( )c7(bL, kn, bAV, vT, bAZ, aW, wu, Lh, aT, Hn, v, Lb, ci, wm, bAW, KY, bAX, bAY); #c1(GDF5) c2(XP_011527378) c3(growth/differentiation factor 5 isoform X1) c4(321984) c5(321985, 321986, 321987, 321988, 321989, 321990, 321991, 321992, 321993, 321994, 321995, 321996, 321997, 321998, 321999, 322000, 322001, 322002, 322003, 322004, 322005, 322006, 322007, 322008, 322009, 322010, 322011, 322012, 322013, 322014, 322015, 322016, 322017, 322018, 322019, 322020, 322021, 322022, 322023, 322024, 322025, 322026, 322027, 322028, 322029, 322030, 322031, 322032, 322033, 322034, 322035, 322036, 322037, 322038, 322039, 322040, 322041, 322042, 322043, 322044, 322045, 322046, 322047, 322048, 322049, 322050, 322051, 322052, 322053, 322054, 322055, 322056, 322057, 322058, 322059, 322060, 322061, 322062, 322063, 322064, 322065, 322066, 322067, 322068, 322069, 322070, 322071, 322072, 322073, 322074, 322075, 322076, 322077, 322078, 322079, 322080, 322081, 322082, 322083, 322084, 322085, 322086) c6( )c7(atx, rV, bBb, zU, bBe, afb, bBf, btl, aT, bBh, bBa, bBc, aW, bBd, Qx, atb, aDQ, eM, ln, ro, aDR, y, VK, bBg); #c1(GDF6) c2(NP_001001557) c3(growth/differentiation factor 6 precursor) c4(18722, 18723, 18724) c5(18725, 18726, 18727, 18728, 18729, 18730, 18731, 18732, 18733, 18734, 18735, 18736, 18737, 18738, 18739, 18740, 18741, 18742) c6( )c7(kn, Na, bAV, jW, W, kj, bAZ, nx, Nc, bBi, Nh, bBk, bBj, bAY, de); #c1(GDF7) c2(NP_878248) c3(growth/differentiation factor 7 preproprotein) c4(257056) c5(257057, 257058, 257059, 257060, 257061, 257062) c6( )c7(cF); #c1(GDF9) c2(XP_011541610) c3(growth/differentiation factor 9 isoform X1) c4(340159) c5(340160, 340161, 340162, 340163, 340164, 340165, 340166, 340167, 340168, 340169, 340170, 340171, 340172, 340/73) c6(340174, 340175, 340176, 340177) c7(T, kl, vJ, asP, aZ, BJ, il, pQ, aT, Uv, aTS, zZ, ae, ad); #c1(GDI1) c2(NP_001484) c3(rab GDP dissociation inhibitor alpha) c4(118070, 118071) c5(118072, 118073, 118074, 118075, 118076, 118077, 118078, 118079, 118080, 118081, 118082, 118083) c6( )c7(AT, bBI, lF, nf, K, nA, PJ, t, bBm); #c1(GDI2) c2(NP_001108628) c3(rab GDP dissociation inhibitor beta isoform 2) c4(43260) c5(43261, 43262, 43263, 43264, 43265, 43266, 43267, 43268, 43269, 43270, 43271, 43272, 43273, 43274, 43275, 43276, 43277) c6( )c7(f, ci, it, K, nA); #c1(GDI2) c2(NP_001485) c3(rab GDP dissociation inhibitor beta isoform 1) c4(118084, 118085, 118086, 118087, 118088, 118089, 118090) c5(118091, 118092, 118093, 118094, 118095, 118096, 118097, 118098, 118099, 118100, 118101, 118102, 118103, 118104, 118105, 118106, 118107, 118108, 118109, 118110, 118111, 118112, 118113, 118114, 118115, 118116, 118117, 118118, 118119, 118120, 118121) c6( )c7(f, ci, it, K, nA); #c1(GDNF) c2(NP_001177397) c3(glial cell line-derived neurotrophic factor isoform 3 preproprotein) c4(72743, 72744) c5(72745, 72746, 72747, 72748, 72749, 72750, 72751, 72752, 72753) c6(72754, 72755) c7(T, aC, aiH, bB, aE, fm, pX, Qk, K, bBp, xa, cp, aW, ahF, as, cm, QA, eM, nB, Ji, U, vy, QF, nw, m, cZ, wu, sw, Ol, My, aj, ct, PJ, JW, eW, wG, bl, dd, he, aMi, dx, hQ, alw, mo, kk, cv, hq, ok, dB, hk, eD, cB, ail, bBo, GC, j, bX, lu, q, J, er, eA, Kx, cF, hb, Qg, aT, cy, rO, bK, an, ahE, ae, af, vT, Y, yL, r, cu, lo, ahl, hf, bu, t, ik, FT, rR, iM, Dy, c, ac, wO, bt, aen, FI, bBn, uO, jh, bS, dG, fu, Fc, iJ, ad, aAq, iC, hd, yt, wm, d, je, alp, xs, Z, adj, bz, eK, Hw, Ew, bE, y); #c1(GDNF) c2(NP_001177398) c3(glial cell line-derived neurotrophic factor isoform 4 preproprotein) c4(72756, 72757, 72758) c5(72759, 72760, 72761, 72762, 72763, 72764, 72765, 72766, 72767, 72768, 72769, 72770) c6(72771, 72772) c7(T, aC, aiH, bB, aE, fm, pX, Qk, K, bBp, xa, cp, aW, ahF, as, cm, DA, eM, nB, Ji, U, vy, QF, nw, m, cZ, wu, sw, Ol, My, aj, ct, PJ, JW, eW, wG, bl, dd, he, aMi, dx, hQ, alw, mo, kk, cv, hq, ok, dB, hk, eD, cB, ail, bBo, GC, j, bX, lu, q, J, er, eA, Kx, cF, hb, Qg, aT, cy, rO, bK, an, ahE, ae, af, vT, Y, yL, r, cu, lo, ahl, hf, bu, t, iK, FT, rR, iM, Dy, c, ac, wl, bt, aen, Fl, bBn, uO, jh, bS, dG, fu, Fc, iJ, ad, aAq, iC, hd, yt, wm, d, je, alp, xs, Z, adj, bz, ek, Hw, Ew, bE, y); #c1(GDNF) c2(NP_954701) c3(glial cell line-derived neurotrophic factor isoform 2 preproprotein) c4( )c5(261298, 261299, 261300, 261301, 261302, 261303, 261304, 261305, 261306, 261307, 261308, 261309, 261310, 261311, 261312, 261313, 261314, 261315, 261316, 261317, 261318, 261319) c6(261320, 261321, 261322, 261323, 261324, 261325) c7(T, aC, aiH, bB, aE, fm, pX, Qk, K, bBp, xa, cp, aW, ahF, as, cm, QA, eM, nB, Ji, U, vy, QF, nw, m, cZ, wu, sw, DI, My, aj, ct, PJ, JW, eW, wG, bl, dd, he, aMi, dx, hQ, alw, mo, kk, cv, hq, ok, dB, hk, eD, cB, ail, bBo, GC, j, bX, lu, q, J, er, eA, Kx, cF, hb, Qg, aT, cy, rO, bK, an, ahE, ae, af, vT, Y, yL, r, cu, lo, ahl, hf, bu, t, ik, FT, rR, iM, Dy, c, ac, wl, bt, aen, Fl, bBn, uO, jh, bS, dG, fu, Fc, iJ, ad, aAq, iC, hd, yt, wm, d, je, alp, xs, Z, adj, bz, ek, Hw, Ew, bE, y); #c1(GDNF) c2(XP_011512331) c3(glial cell line-derived neurotrophic factor isoform X1) c4( )c5(302886, 302887, 302888, 302889, 302890, 302891, 302892, 302893, 302894, 302895, 302896, 302897, 302898, 302899, 302900, 302901, 302902, 302903, 302904, 302905, 302906, 302907, 302908, 302909, 302910, 302911, 302912, 302913, 302914, 302915, 302916, 302917, 302918, 302919, 302920, 302921, 302922, 302923, 302924, 302925, 302926, 302927, 302928, 302929, 302930, 302931, 302932, 302933, 302934, 302935, 302936, 302937, 302938, 302939, 302940, 302941, 302942, 302943, 302944, 302945, 302946, 302947, 302948, 302949, 302950, 302951, 302952, 302953, 302954, 302955, 302956, 302957, 302958, 302959, 302960, 302961, 302962, 302963, 302964, 302965, 302966, 302967, 302968, 302969, 302970, 302971, 302972, 302973, 302974, 302975, 302976, 302977, 302978, 302979) c6(302980, 302981, 302982) c7(T, aC, aiH, bB, aE, fm, pX, Qk, K, bBp, xa, cp, aW, ahF, as, cm, QA, eM, nB, Ji, U, vy, QF, nw, m, cZ, wu, sw, Ol, My, aj, ct, PJ, JW, eW, wG, bl, dd, he, aMi, dx, hQ, alw, mo, kk, cv, hq, ok, dB, hk, eD, cB, ail, bBo, GC, j, bX, lu, q, J, er, eA, Kx, cF, hb, Qg, aT, cy, rO, bK, an, ahE, ae, af, vT, Y, yL, r, cu, lo, ahl, hf, bu, t, iK, FT, rR, iM, Dy, c, ac, wl, bt, aen, Fl, bBn, uO, jh, bS, dG, fu, Fc, iJ, ad, aAq, iC, hd, yt, wm, d, je, alp, xs, Z, adj, bz, ek, Hw, Ew, bE, y); #c1(GDPD3) c2(NP_077283) c3(glycerophosphodiester phosphodiesterase domain-containing protein 3) c4(214908, 214909) c5(214910, 214911, 214912, 214913, 214914, 214915, 214916, 214917, 214918, 214919, 214920, 214921) c6( )c7(du); #c1(GDPD5) c2(NP_110419) c3(glycerophosphodiester phosphodiesterase domain-containing protein 5) c4(220199, 220200) c5(220201, 220202, 220203, 220204, 220205, 220206, 220207, 220208, 220209, 220210, 220211, 220212, 220213, 220214, 220215, 220216, 220217, 220218, 220219, 220220, 220221, 220222, 220223, 220224, 220225, 220226, 220227, 220228, 220229, 220230, 220231, 220232, 220233, 220234, 220235, 220236, 220237) c6( )c7(cm, aW, T, aT); #c1(GDPD5) c2(XP_011543581) c3(glycerophosphodiester phosphodiesterase domain-containing protein 5 isoform X2) c4(342497) c5( )c6( )c7(cm, aW, T, aT); #c1(GDPD5) c2(XP_011543582) c3(glycerophosphodiester phosphodiesterase domain-containing protein 5 isoform X3) c4( ) c5(342498) c6( )c7(cm, aW, T, aT); #c1(GDPD5) c2(XP_011543587) c3 (glycerophosphodiester phosphodiesterase domain-containing protein 5 isoform X5) c4(342499, 342500, 342501) c5(342502) c6( )c7(cm, aW, T, aT); #c1(GDPD5) c2(XP_011543589) c3 (glycerophosphodiester phosphodiesterase domain-containing protein 5 isoform X7) c4( )c5(342503, 342504, 342505, 342506, 342507) c6( )c7(cm, aW, T, aT); #c1(GEM) c2(NP_859053) c3(GTP-binding protein GEM) c4(255409, 255410) c5(255411, 255412, 255413, 255414, 255415, 255416, 255417, 255418, 255419, 255420, 255421, 255422, 255423, 255424, 255425, 255426) c6( )c7(bL, T, jv, axd, al, jx, jh, fd, n, aeu, aj, uu, jE, v, lR, fr, V, mC, amW, J, aMp, hX, bBq, AW, x, F, wD, akU, ld, gd, aT, dV, h, 1G, hH, A, mR, iU, aW, Y, aFE, WQ, HA, eZ, lo, hp, bw, bz, pb, dr, aYt, bN, ck, iP, gk, W, iT, Jr, aWZ, ig, dW, oX, k, yH, fl, pw, eo, bt, fh, B, y, vi); #c1 (GEMIN2) c2(NP_001009182) c3(gem-associated protein 2 isoform beta) c4(23517) c5(23518, 23519, 23520, 23521, 23522, 23523) c6( )c7(T, aAf, Ol, aA, bB, fr, ci, eA, aE, Tf, kf, h, hH, ahE, ff, aq, aF, ahl, en, ik, c, eM, bE, m, B, hv, hS); #c1(GEMIN2) c2(NP_001009183) c3(gem-associated protein 2 isoform gamma) c4(23524) c5(23525, 23526, 23527, 23528, 23529, 23530) c6( )c7(T, aAf, Ol, aA, bB, fr, ci, eA, aE, Tf, kf, h, hH, ahE, ff, aq, aF, ahl, en, ik, c, eM, bE, m, B, hv, hS); #c1(GEMIN2) c2(NP_003607) c3(gem-associated protein 2 isoform alpha) c4(139543) c5(139544, 139545, 139546, 139547, 139548, 139549) c6( )c7(T, aAf, Ol, aA, bB, fr, ci, eA, aE, Tf, kf, h, hH, ahE, ff, aq, aF, ahl, en, ik, c, eM, bE, m, B, hv, hS); #c1(GEMIN4) c2(NP_056536) c3(gem-associated protein 4) c4(187994, 187995) c5(187996, 187997, 187998, 187999, 188000, 188001, 188002, 188003, 188004, 188005, 188006) c6( )c7(T, if, ht, hy, fr, ci, qN, oj, aT, kf, aU, dL, hP, K, hu, eL, af, B, BN, aW, Y, aq, iR, es, bb, c, Kv, U, it, ne); #c1(GEMIN4) c2(XP_011522215) c3(gem-associated protein 4 isoform X2) c4( )c5(316842) c6( )c7(T, if, ht, hy, fr, ci, qN, oj, aT, kf, aU, dL, hP, K, hu, eL, af, B, BN, aW, Y, aq, iR, es, bb, c, Kv, U, it, ne); #c1(GEMIN6) c2(NP_079051) c3(gem-associated protein 6) c4(216746, 216747) c5(216748, 216749, 216750, 216751, 216752, 216753, 216754, 216755, 216756, 216757, 216758) c6( )c7(kf, 01); #c1(GEMIN6) c2(XP_005264625) c3(gem-associated protein 6 isoform X1) c4( )c5(279152) c6( )c7(kf, 01); #c1(GEN1) c2(NP_872431) c3(flap endonuclease GEN homolog 1) c4(256344, 256345, 256346, 256347) c5(256348, 256349, 256350, 256351, 256352, 256353, 256354, 256355, 256356, 256357, 256358, 256359, 256360, 256361, 256362, 256363, 256364, 256365, 256366, 256367, 256368, 256369, 256370, 256371, 256372, 256373, 256374, 256375) c6( )c7(T, dB, aW, aT, m, t, ae, eA, ad); #c1(GET4) c2(NP_057033) c3(Golgi to ER traffic protein 4 homolog) c4(188570) c5(188571, 188572, 188573, 188574, 188575, 188576) c6( )c7(bX); #c1(GFAP) c2(NP_001124491) c3(glial fibrillary acidic protein isoform 2) c4(49151) c5(49152, 49153, 49154, 49155, 49156, 49157, 49158, 49159, 49160, 49161, 49162) c6( )c7(T, aiH, Tb, alW, aR, ip, aao, aEl, Hr, ku, fm, K, H, Bp, eG, aW, Ls, as, eF, kJ, apB, jV, Kv, wK, dc, ki, mx, U, nw, mk, act, gD, cZ, Ol, aw, qy, aj, asF, PJ, eW, ke, aBy, h, dd, he, vx, Ha, pi, Lg, nP, A, bux, ack, dw, cA, dB, aWn, hR, HB, eo, arO, B, L, ka, bBr, GC, OY, bX, V, eA, J, bBs, hb, WY, aT, cy, rO, bK, an, qk, af, eu, jf, ak, Uf, la, qL, en, f, rY, dS, vk, Di, tj, c, Kg, On, kh, bqP, aaz, ha, jN, bS, dG, Fc, afb, hX, iC, sO, oj, acd, cb, NM, iL, d, lR, ff, aGS, aJK, ek, D, Ew, auH, Hs, xg, jr); #c1(GFAP) c2(NP_001229305) c3(glial fibrillary acidic protein isoform 3) c4(84191) c5(84192, 84193, 84194, 84195, 84196, 84197, 84198) c6( )c7(T, aiH, Tb, alW, aR, ip, aao, aEl, Hr, ku, fm, K, H, Bp, eG, aW, Ls, as, eF, kJ, apB, jV, Kv, wK, dc, ki, mx, U, nw, mk, act, gD, cZ, Ol, aw, qy, aj, asF, PJ, eW, ke, aBy, h, dd, he, vx, Ha, pi, Lg, nP, A, bux, ack, dw, cA, dB, aWn, hR, HB, eo, arD, B, L, ka, bBr, GC, OY, bX, V, eA, J, bBs, hb, WY, aT, cy, rO, bK, an, qk, af, eu, jf, ak, Uf, la, qL, en, f, rY, dS, vk, Qi, tj, c, Kg, On, kh, bqP, aaz, ha, jN, bS, dG, Fc, afb, hX, iC, sO, oj, acd, cb, NM, iL, d, lR, ff, aGS, aJK, ek, D, Ew, auH, Hs, xg, jr); #c1(GFAP) c2(NP_002046) c3(glial fibrillary acidic protein isoform 1) c4(124058, 124059, 124060) c5(124061, 124062, 124063, 124064, 124065, 124066, 124067, 124068, 124069, 124070, (24071, (24072) c6( )c7(T, aiH, Tb, alW, aR, ip, aao, aEl, Hr, ku, fm, K, H, Bp, eG, aW, Ls, as, eF, kJ, apB, jV, Kv, wK, dc, ki, mx, U, nw, mk, act, gD, cZ, Ol, aw, qy, aj, asF, PJ, eW, ke, aBy, h, dd, he, vx, Ha, pi, Lg, nP, A, bux, ack, dw, cA, dB, aWn, hR, HB, eo, arO, B, L, ka, bBr, GC, OY, bX, V, eA, J, bBs, hb, WY, aT, cy, rO, bk, an, qk, af, eu, jf, ak, Uf, la, qL, en, f, rY, dS, vk, Qi, tj, c, Kg, On, kh, bqP, aaz, ha, jN, bS, dG, Fc, afb, hX, iC, sO, oj, acd, cb, NM, iL, d, lR, ff, aGS, aJK, ek, D, Ew, auH, Hs, xg, jr); #c1(GFER) c2(NP_005253) c3(FAD-linked sulfhydryl oxidase ALR) c4(154401, 154402) c5(154403, 154404, 154405, 154406, 154407, 154408, 154409, 154410, 154411, 154412, 154413, 154414) c6( )c7(T, al, jx, aj, ci, C, Lw, bX, eA, J, hX, x, jz, oj, cy, K, jo, yi, A, bBt, hl, aq, bBu, kC, KM, gk, mG, vy, E, k, B); #c1(GFI1) c2(XP_011539548) c3(zinc finger protein Gfi-1 isoform X1) c4(337742) c5(337743, 337744, 337745, 337746, 337747, 337748, 337749, 337750, 337751, 337752, 337753, 337754, 337755, 337756, 337757) c6( )c7(gV, aKm, jh, fd, afK, ci, aoC, V, ad, br, bly, x, ft, zw, h, hn, ae, aXz, jf, bBv, hG, bBw, aq, bw, bz, ck, KB, k, bt); #c1(GFI1B) c2(NP_001128503) c3(zinc finger protein Gfi-lb isoform 2) c4(50107) c5(50108, 50109, 50110, 50111, 50112, 50113, 50114, 50115, 50116, 50117, 50118, 50119, 50120, 50121, 50122) c6( ) c7(x, jf, T, bBx, il, jh, pg, iY, dX, bw, bt); #c1(GFI1B) c2(NP_004179) c3(zinc finger protein Gfi-lb isoform 1) c4(144119) c5(144120, 144121, 144122, 144123, 144124, 144125, 144126, 144127, 144128, 144129, 144130, 144131, 144132, 144133, 144134) c6( )c7(x, jf, T, bBx, iU, jh, pg, iY, dx, bw, bt); #c1(GFM1) c2(NP_079272) c3(elongation factor G, mitochondrial) c4(217650, 217651) c5(217652, 217653, 217654, 217655, 217656, 217657) c6( )c7(bBy, bBz, hb, aW, aT, wR, kC, ajU); #c1(GFPT1) c2(NP_001231639) c3(glutamine-fructose-6-phosphate aminotransferase [isomerizing] 1 isoform 1) c4(87376) c5(87377, 87378, 87379, 87380, 87381, 87382) c6( )c7(bL, aC, xA, aE, dB, aDx, aeu, aj, dy, bBB, bBA, bq, dl, v, jy, J, BA); #c1(GFPT1) c2(NP_002047) c3(glutamine-fructose-6-phosphate aminotransferase [isomerizing] 1 isoform 2) c4(124073) c5(124074, 124075, 124076, 124077, 124078, 124079) c6( )c7(bL, aC, xA, aE, dB, aDx, aeu, aj, dy, bBB, bBA, bq, dQ, v, jy, J, BA); #c1(GFPT2) c2(NP_005101) c3(glutamine-fructose-6-phosphate aminotransferase [isomerizing] 2) c4(152881) c5(152882, 152883, 152884, 152885, 152886, 152887) c6( )c7(bL, fU, aw, an); #c1(GFRA1) c2(NP_001138925) c3(GDNF family receptor alpha-l isoform b preproprotein) c4(58222, 58223) c5(58224, 58225, 58226, 58227, 58228, 58229, 58230, 58231, 58232, 58233, 58234, 58235, 58236, 58237, 58238, 58239, 58240, 58241, 58242, 58243) c6( )c7(cZ, DI, My, jx, ul, fu, bX, eA, Kx, HF, hd, aE, wG, nv, pX, aT, cy, ro, dd, ahE, eu, EV, Y, aW, vj, aq, HL, ahl, Hw, ac, c, nB, QF, Lb, B, DB); #c1(GFRAI) c2(XP_011537936) c3 (GDNF family receptor alpha-isoform X1) c4(335444) c5(335445, 335446, 335447, 335448, 335449, 335450) c6( )c7(cZ, Ol, My, jx, uO, fu, bX, eA, Kx, HF, hd, aE, wG, nv, pX, aT, cy, rO, dd, ahE, eu, EV, Y, aW, vj, aq, HL, ahl, Hw, ac, c, nB, QF, Lb, B, DB); #c1(GFRA2) c2(NP_001158511) c3(GDNF family receptor alpha-2 isoform c precursor) c4(63699) c5(63700, 63701, 63702, 63703, 63704, 63705) c6( )c7(HF, Hw, hd, aE, bBC, fu, ahl, ahE, af, cv); #c1(GFRA2) c2(XP_011542786) c3(GDNF family receptor alpha-2 isoform X2) c4(341392, 341393) c5(341394, 341395, 341396, 341397, 341398, 341399, 341400, 341401, 341402, 341403, 341404, 341405, 341406, 341407, 341408, 341409) c6( )c7(HF, Hw, hd, aE, bBC, fu, ahl, ahE, af, cv); #c1(GFRA2) c2(XP_011542787) c3(GDNF family receptor alpha-2 isoform X1) c4(341410) c5(341411, 341412, 341413, 341414, 341415, 341416) c6( )c7(HF, Hw, hd, aE, bBC, fu, ahl, ahE, af, cv); #c1(GFRA3) c2(NP_001487) c3(GDNF family receptor alpha-3 preproprotein) c4(118122, 118123) c5(118124, 118125, 118126, 118127, 118128, 118129, 118130, 118131, 118132, 118133, 118134, 118135) c6( )c7(aW, hd, My, eR, eM); #c1(GFRA4) c2(NP_071422) c3(GDNF family receptor alpha-4 isoform a precursor) c4(211579) c5(211580, 211581, 211582, 211583, 211584, 211585, 211586, 211587) c6(211588, 211589) c7(bxy, bM, ahE, ahl); #c1(GFRA4) c2(NP_665705) c3(GDNF family receptor alpha-4 isoform b precursor) c4(241680) c5(241681, 241682, 241683, 241684, 241685, 241686, 241687, 241688) c6(241689, 241690) c7(bxy, bM, ahE, ahl); #c1(GGA1) c2(NP_001001560) c3(ADP-ribosylation factor-binding protein GGA1 isoform 2) c4(18743) c5(18744, 18745, 18746, 18747, 18748, 18749, 18750, 18751, 18752, 18753, 18754, 18755, 18756, 18757, 18758, 18759, 18760, 18761, 18762, 18763, 18764, 18765, 18766, 18767, 18768, 18769, 18770, 18771, 18772, 18773, 18774, 18775, 18776, 18777, 18778, 18779, 18780, 18781, 18782, 18783, 18784, 18785, 18786, 18787, 18788, 18789, 18790) c6( )c7(af); #c1(GGA1) c2(NP_001166158) c3(ADP-ribosylation factor-binding protein GGA1 isoform 4) c4(68378) c5( )c6( )c7(af); #c1(GGA1) c2(NP_037497) c3(ADP-ribosylation factor-binding protein GGA1 isoform 1) c4(175845, 175846) c5(175847, 175848, 175849, 175850, 175851, 175852, 175853, 175854, 175855, 175856, 175857, 175858) c6( )c7(af); #c1(GGA1) c2(XP_005261575) c3(ADP-ribosylation factor-binding protein GGA1 isoform X4) c4(277483) c5(277484, 277485, 277486, 277487, 277488, 277489, 277490, 277491) c6( )c7(af); #c1(GGA1) c2(XP_011528424) c3(ADP-ribosylation factor-binding protein GGA1 isoform X2) c4(323538, 323539) c5(323540, 323541, 323542, 323543, 323544, 323545, 323546, 323547, 323548, 323549, 323550, 323551, 323552, 323553, 323554, 323555) c6( )c7(af); #c1(GGA1) c2(XP_011528425) c3(ADP-ribosylation factor-binding protein GGA1 isoform X3) c4(323556, 323557) c5( )c6( )c7(af); #c1(GGA1) c2(XP_011528426) c3(ADP-ribosylation factor-binding protein GGA1 isoform X5) c4( )c5(323558, 323559, 323560, 323561, 323562, 323563) c6( )c7(af); #c1(GGA1) c2(XP_011528427) c3(ADP-ribosylation factor-binding protein GGA1 isoform X1) c4(323564, 323565, 323566, 323567, 323568, 323569) c5(323570, 323571, 323572, 323573, 323574) c6( )c7(af); #c1(GGA3) c2(NP_001166175) c3(ADP-ribosylation factor-binding protein GGA3 isoform 2) c4(68435) c5(68436, 68437, 68438, 68439, 68440) c6( )c7(af, eu); #c1(GGA3) c2(NP_001278570) c3(ADP-ribosylation factor-binding protein GGA3 isoform 1) c4( )c5(109855, 109856, 109857, 109858, 109859, 109860, 109861, 109862, 109863, 109864, 109865, 109866, 109867, 109868) c6( )c7(af, eu); #c1(GGA3) c2(NP_054720) c3(ADP-ribosylation factor-binding protein GGA3 isoform short) c4(177342) c5(177343, 177344, 177345, 177346, 177347, 177348, 177349) c6( )c7(af, eu); #c1(GGA3) c2(NP_619525) c3(ADP-ribosylation factor-binding protein GGA3 isoform 1ong) c4(236801, 236802, 236803) c5(236804, 236805, 236806, 236807, 236808, 236809, 236810, 236811, 236812, 236813, 236814, 236815, 236816) c6( ) c7(af, eu); #c1(GGA3) c2(XP_011522865) c3(ADP-ribosylation factor-binding protein GGA3 isoform X1) c4(317347) c5(317348) c6( ) c7(af, eu); #c1(GGACT) c2(NP_149101) c3(gamma-glutamylaminecyclotransferase) c4(227154) c5(227155, 227156, 227157, 227158, 227159, 227160, 227161, 227162, 227163, 227164) c6( )c7(bM); #c1(GGCT) c2(NP_001186744) c3(gamma-glutamylcyclotransferase isoform 2) c4(78489) c5(78490, 78491, 78492, 78493, 78494, 78495) c6( )c7(T, gU, hV, iB, aA, ci, ai, ad, bv, av, rC, aT, vH, h, ey, rz, on, ae, yi, Ln, s, Cw, aW, Lf, eH, eZ, aq, aF, Ku, bu, g, t, eF, bz, pb, ik, bN, dR, ck, qe, qr, ZT, hR, m, ER, aed, alc); #c1(GGCT) c2(NP_001186745) c3(gamma-glutamylcyclotransferase isoform 3) c4(78496) c5(78497, 78498, 78499, 78500, 78501, 78502, 78503, 78504) c6( )c7(T, gU, hV, iB, aA, ci, ai, ad, bv, av, rC, aT, vH, h, ey, rz, on, ae, yi, Ln, s, Cw, aW, Lf, eH, eZ, aq, aF, Ku, bu, g, t, eF, bz, pb, ik, bN, dR, ck, qe, qr, ZT, hR, m, ER, aed, aJc); #c1(GGCT) c2(NP_001186746) c3(gamma-glutamylcyclotransferase isoform 4) c4(78505) c5(78506, 78507, 78508, 78509, 78510, 78511) c6( )c7(T, gU, hV, iB, aA, ci, ai, ad, bv, av, rC, aT, vH, h, ey, rz, on, ae, yi, Ln, s, Cw, aW, Lf, eH, eZ, aq, aF, Ku, bu, g, t, eF, bz, pb, ik, bN, dR, ck, qe, qr, ZT, hR, m, ER, aed, alc); #c1(GGCT) c2(NP_076956) c3(gamma-glutamylcyclotransferase isoform 1) c4(214457, 214458) c5(214459, 214460, 214461, 214462, 214463, 214464, 214465, 214466, 214467, 214468, 214469, 214470) c6( ) c7(T, gU, hV, iB, aA, ci, ai, ad, bv, av, rC, aT, vH, h, ey, rz, on, ae, yi, Ln, s, Cw, aW, Lf, eH, eZ, aq, aF, Ku, bu, g, t, eF, bz, pb, iK, bN, dR, ck, qe, qr, ZT, hR, m, ER, aed, alc); #c1(GGCX) c2(NP_000812) c3(vitamin K-dependent gamma-carboxylase isoform 1) c4(16325) c5(16326, 16327, 16328, 16329, 16330, 16331) c6( )c7(dD, bBD, ac, bpN, bBE, dX, eq, bqA, aOh, aaD, fl, ad); #c1(GGCX) c2(NP_001135741) c3(vitamin K-dependent gamma-carboxylase isoform 2) c4(52963) c5(52964, 52965, 52966, 52967, 52968, 52969) c6( )c7(dD, bBD, ac, bpN, bBE, dX, eq, bqA, ah, aaD, fl, ad); #c1(GGH) c2(NP_003869) c3(gamma-glutamyl hydrolase precursor) c4(141646) c5(141647, 141648, 141649, 141650, 141651, 141652) c6( )c7(T, if, e, cl, ci, qN, q, x, pX, aT, dL, ln, eG, d, aW, ff, cr, r, aq, en, g, iV, cM, ck, lp, at, k, y, B); #c1 (GGN) c2(NP_689870) c3(gametogenetin) c4(244225, 244226) c5(244227, 244228, 244229, 244230, 244231, 244232, 244233, 244234, 244235, 244236, 244237, 244238) c6( )c7(T, yi, gU, id, adZ, aW, Lf, hL, aT, adX, PD, Pp, v, ei, cl, ae, ad); #c1(GGN) c2(XP_011524905) c3(gametogenetin isoform X2) c4( )c5(319305, 319306) c6( )c7(T, yi, gU, id, adZ, aW, Lf, hL, aT, adX, PD, Pp, v, ei, cl, ae, ad); #c1(GGNBP2) c2(NP_079 μl) c3(gametogenetin-binding protein 2) c4(217077) c5(217078, 217079, 217080, 217081, 217082, 217083) c6( ) c7(cm, T, ci, rA); #c1 (GGPS1) c2(NP_001032354) c3 (geranylgeranyl pyrophosphate synthase) c4(31207, 31208, 31209) c5(31210, 31211, 31212, 31213, 31214, 31215, 31216, 31217, 31218, 31219, 31220, 31221, 31222, 31223, 31224, 31225, 31226, 31227, 31228, 31229, 31230, 31231, 31232, 31233, 31234, 31235, 31236, 31237, 31238, 31239) c6( )c7(gk, al, r, n, jy, aq, q, iR, B); #c1(GGT1) c2(NP_038347) c3(gamma-glutamyltranspeptidase 1 precursor) c4(176369, 176370, 176371, 176372, 176373, 176374, 176375, 176376, 176377, 176378, 176379, 176380, 176381, 176382, 176383, 176384) c5(176385, 176386, 176387, 176388, 176389, 176390, 176391, 176392, 176393, 176394, 176395, 176396, 176397, 176398, 176399, 176400, 176401, 176402, 176403, 176404, 176405, 176406, 176407, 176408, 176409, 176410, 176411, 176412, 176413, 176414, 176415, 176416, 176417, 176418, 176419, 176420, 176421, 176422, 176423, 176424, 176425, 176426, 176427, 176428, 176429, 176430, 176431, 176432, 176433, 176434, 176435, 176436, 176437, 176438, 176439, 176440, 176441, 176442, 176443, 176444, 176445, 176446, 176447, 176448, 176449, 176450, 176451, 176452, 176453, 176454, 176455, 176456, 176457, 176458, 176459, 176460, 176461, 176462, 176463, 176464, 176465, 176466, 176467, 176468, 176469, 176470, 176471, 176472, 176473, 176474, 176475, 176476, 176477, 176478, 176479, 176480, 176481, 176482, 176483, 176484, 176485, 176486, 176487, 176488, 176489) c6( )c7(T, aC, al, fd, ame, ci, ip, aJM, x, Vp, aE, dV, dQ, aPB, aci, zQ, az, hl, fc, aB, g, hJ, bw, eM, k, pw, ik, wu, aw, bzb, asM, n, aj, nA, zP, rc, rC, Nf, OV, A, bBF, aq, bBG, vD, uz, eo, el, bD, B, eR, yk, jx, aOH, cz, ti, v, aU, V, q, J, hW, qK, bq, il, Y, als, en, cl, t, kC, ck, BU, nM, aor, aU, mf, bt, akd, qb, uG, jh, ad, F, aop, db, wm, MG, ff, eZ, EH, bz, bF, gk, bnn, qe, kq, cP, be, ei); #c1(GGT2) c2(XP_011528923) c3(inactive gamma-glutamyltranspeptidase 2 isoform XII) c4( )c5(324286, 324287, 324288, 324289, 324290, 324291, 324292, 324293, 324294, 324295, 324296, 324297) c6( )c7(T, aC, akd, wu, yk, uG, al, jx, jh, n, aj, ci, q, J, ad, aJM, x, F, aE, hW, rC, qK, Nf, wm, dQ, OV, als, az, fc, eZ, aq, aB, cl, bBG, hJ, kC, bz, gk, nM, vD, eM, uz, k, cP, eQ, bt, B); #c1(GGT2) c2(XP_011528924) c3(inactive gamma-glutamyltranspeptidase 2 isoform X12) c4(324298, 324299) c5(324300, 324301, 324302, 324303, 324304, 324305, 324306, 324307, 324308, 324309, 324310, 324311, 324312, 324313, 324314, 324315) c6( )c7(T, aC, akd, wu, yk, uG, al, jx, jh, n, aj, ci, q, J, ad, aJM, x, F, aE, hW, rC, qK, Nf, wm, Qe, OV, als, az, fc, eZ, aq, aB, cl, bBG, hJ, kC, bz, gk, nM, vD, eM, uz, k, cP, eQ, bt, B); #c1(GGT5) c2(NP_001093251) c3(gamma-glutamyltransferase 5 isoform 1) c4(40163) c5(40164, 40165, 40166, 40167, 40168, 40169) c6( )c7(aq, av, B); #c1(GGT5) c2(NP_001093252) c3(gamma-glutamyltransferase 5 isoform 3) c4(40170) c5(40171, 40172, 40173, 40174, 40175, 40176) c6( )c7(aq, av, B); #c1(GGT5) c2(NP_001289394) c3(gamma-glutamyltransferase 5 isoform 5) c4(114/78) c5(114/79, 114180, 114181, 114182, 114183, 114184) c6( )c7(aq, av, B); #c1(GGT5) c2(NP_004112) c3(gamma-glutamyltransferase 5 isoform 2) c4(143662, 143663) c5(143664, 143665, 143666, 143667, 143668, 143669, 143670, 143671, 143672, 143673, 143674, 143675) c6( )c7(aq, av, B); #c1(GGTLC1) c2(XP_005260922) c3(gamma-glutamyltransferase light chain 1 isoform X1) c4(276972) c5(276973, 276974, 276975, 276976, 276977, 276978, 276979, 276980, 276981, 276982, 276983) c6( )c7(afk, uG, cz, jw, l, ab, h, H, ae, hG, az, hl, eZ, fc, aq, bBH, aB, hJ, afj, jq, aOL, bjk, cP, bBI, B); #c1(GGTLC1) c2(XP_006723725) c3(gamma-glutamyltransferase light chain 1 isoform X4) c4( )c5(295326, 295327, 295328, 295329) c6( )c7(afk, uG, cz, jw, l, ab, h, H, ae, hG, az, hl, eZ, fc, aq, bBH, aB, hJ, afj, jq, aOL, bjk, cP, bBI, B); #c1(GH1) c2(NP_000506) c3(somatotropin isoform 1 precursor) c4(10578, 10579, 10580, 10581, 10582) c5(10583, 10584, 10585, 10586, 10587, 10588, 10589, 10590, 10591, 10592, 10593, 10594, 10595, 10596, 10597, 10598, 10599, 10600, 10601, 10602, 10603, 10604, 10605, 10606, 10607, 10608, 10609, 10610, 10611, 10612, 10613, 10614, 10615, 10616, 10617, 10618, 10619, 10620, 10621, 10622, 10623, 10624, 10625, 10626, 10627, 10628, 10629, 10630, 10631, 10632, 10633, 10634, 10635, 10636, 10637, 10638, 10639, 10640, 10641, 10642, 10643, 10644, 10645, 10646, 10647, 10648, 10649, 10650, 10651, 10652, 10653, 10654, 10655, 10656, 10657, 10658, 10659, 10660, 10661, 10662, 10663, 10664, 10665, 10666, 10667, 10668, 10669, 10670, 10671, 10672, 10673, 10674, 10675, 10676, 10677, 10678, 10679, 10680, 10681) c6(10682, 10683, 10684) c7(oO, T, aC, ahS, bBK, vf, ea, e, al, yS, acz, bBR, amj, ci, aY, ku, tg, wM, aX, vg, K, EL, qM, dl, pk, blc, aVB, eL, bBQ, yo, ZH, aW, Fe, hl, fc, g, eF, avx, jV, nB, lq, vA, mx, vy, CD, aYS, m, R, amz, bBP, Ht, bBJ, uf, dt, dD, lT, Ol, wu, aw, ht, n, cQ, aj, kM, bBS, ml, zP, JW, cd, rc, biN, jL, rC, h, jy, eV, dL, bl, uV, Df, awV, avP, A, CN, EU, Rj, fQ, ahJ, aq, fe, OB, bmd, op, asE, bo, mg, hL, hR, HB, arg, B, AN, bL, AB, eR, Dh, eX, qQ, cz, adi, asA, v, amC, avQ, q, J, UE, oT, mj, XD, aql, aT, bBN, rD, zp, rv, ae, af, bA, kl, bnk, abs, s, vT, Y, asy, LO, r, akY, bu, cl, t, f, bQ, bBL, aDO, dS, bN, BU, acW, tW, ac, rR, mG, mf, dy, aDR, aoJ, UF, gT, xb, bBM, pY, Oy, acl, iW, dG, oH, eq, aXD, ad, qC, fU, dP, vJ, qh, ji, wm, DI, lR, PE, baT, xs, bBO, eH, cr, nf, jg, dr, bdu, dR, gk, OG, Ew, ll, sG, aXJ, cP, bC, ei, y, jr); #c1(GH1) c2(NP_072053) c3(somatotropin isoform 2 precursor) c4(212620) c5(212621, 212622, 212623, 212624, 212625, 212626, 212627, 212628, 212629, 212630, 212631) c6(212632, 212633) c7(oO, T, aC, ahS, bBK, vf, ea, e, al, yS, acz, bBR, amj, ci, aY, ku, tg, wM, aX, vg, K, EL, qM, Qe, pk, blc, aVB, eL, bBQ, yo, ZH, aW, Fe, hl, fc, g, eF, avx, jV, nB, lq, vA, mx, vy, CD, aYS, m, R, amz, bBP, Ht, bBJ, uf, dt, dD, lT, Ol, wu, aw, ht, n, cQ, aj, kM, bBS, ml, zP, JW, cd, rc, biN, jL, rC, h, jy, eV, dL, bl, uV, Df, awV, avP, A, CN, EU, Rj, fQ, ahJ, aq, fe, OB, bmd, op, asE, bo, mg, hL, hR, HB, arg, B, AN, bL, AB, eR, Dh, eX, qQ, cz, adi, asA, v, amC, avO, q, J, UE, oT, mj, XD, aql, aT, bBN, rD, zp, rv, ae, af, bA, kl, bnk, abs, s, vT, Y, asy, LO, r, akY, bu, cl, t, f, bQ, bBL, aDQ, dS, bN, BU, acW, tW, ac, rR, mG, mf, dy, aDR, aoJ, UF, gT, xb, bBM, pY, Oy, acl, iW, dG, oH, eq, aXD, ad, qC, fU, dP, vJ, qh, ji, wm, DI, lR, PE, bal, xs, bBO, eH, cr, nf, jg, dr, bdu, dR, gk, OG, Ew, ll, sG, aXJ, cP, bC, ei, y, jr); #c1(GH1) c2(NP_072054) c3(somatotropin isoform 3 precursor) c4(212634) c5(212635, 212636, 212637, 212638, 212639, 212640, 212641, 212642) c6(212643, 212644) c7(oO, T, aC, ahS, bBK, vf, ea, e, al, yS, acz, bBR, amj, ci, aY, ku, tg, wM, aX, vg, K, EL, qM, Qe, pk, blc, aVB, eL, bBQ, yo, ZH, aW, Fe, hl, fc, g, eF, avx, jV, nB, lq, vA, mx, vy, CD, aYS, m, R, amz, bBP, Ht, bBJ, uf, dt, dD, lT, Ol, wu, aw, ht, n, cl, aj, kM, bBS, ml, zP, JW, cd, rc, biN, jL, rC, h, jy, eV, dL, bl, uV, Df, awV, avP, A, CN, EU, Rj, fQ, ahJ, aq, fe, OB, bmd, op, asE, bo, mg, hL, hR, HB, arg, B, AN, bL, AB, eR, Dh, eX, qQ, cz, adi, asA, v, amC, avO, q, J, UE, oT, mj, XD, aql, aT, bBN, rD, zp, rv, ae, af, bA, kl, bnk, abs, s, vT, Y, asy, LO, r, akY, bu, cl, t, f, bQ, bBL, aDO, dS, bN, BU, acW, tW, ac, rR, mG, mf, dy, aDR, aoJ, UF, gT, xb, bBM, pY, Oy, acl, iW, dG, OH, eq, aXD, ad, qC, fU, dP, vJ, qh, ji, wm, DI, lR, PE, baT, xs, bBO, eH, cr, nf, jg, dr, bdu, dR, gk, OG, Ew, ll, sG, aXJ, cP, bC, ei, y, jr); #c1(GH2) c2(NP_002050) c3(growth hormone variant isoform 1 precursor) c4(124080) c5(124081, 124082, 124083, 124084, 124085, 124086, 124087, 124088) c6(124089, 124090) c7(bL, T, wu, ht, jh, v, ci, wm, nJ, s, vT, Lf, LO, akY, g, bu, uC, mg, hR, bko, ei, yv); #c1(GHZ) c2(NP_072050) c3(growth hormone variant isoform 4 precursor) c4(212587) c5(212588, 212589, 212590, 212591, 212592, 212593, 212594, 212595) c6(212596, 212597) c7(bL, T, wu, ht, jh, v, ci, wm, nJ, s, vT, Lf, LO, akY, g, bu, uC, mg, hR, bko, ei, yv); #c1(GH2) c2(NP_072051) c3(growth hormone variant isoform 2 precursor) c4(212598) c5(212599, 212600, 212601, 212602, 212603, 212604, 212605, 212606) c6(212607, 212608) c7(bL, T, wu, ht, jh, v, ci, wm, nJ, s, vT, Lf, LO, akY, g, bu, uC, mg, hR, bko, ei, yv); #c1 (GH2) c2(NP_072052) c3 (growth hormone variant isoform 3 precursor) c4(212609) c5(212610, 212611, 212612, 212613, 212614, 212615, 212616, 212617) c6(212618, 212619) c7(bL, T, wu, ht, jh, v, ci, wm, nJ, s, vT, Lf, LO, akY, g, bu, uC, mg, hR, bko, ei, yv); #c1(GHITM) c2(NP_055209) c3(growth hormone-inducible transmembrane protein) c4(179826, 179827, 179828, 179829) c5(179830, 179831, 179832, 179833, 179834, 179835, 179836, 179837, 179838, 179839, 179840, 179841, 179842, 179843, 179844, 179845, 179846, 179847, 179848, 179849, 179850, 179851, 179852, 179853) c6( )c7(hy, ne, BN); #c1(GHR) c2(NP_001229328) c3(growth hormone receptor isoform 2 precursor) c4( )c5(84213) c6( )c7(T, pf, aoq, zs, e, al, atj, if, ci, ip, yo, aW, hl, LZ, g, eF, vA, ZT, bBU, OD, m, bBT, FG, fg, aj, JW, rc, adf, ec, dL, bl, avP, A, Rj, iw, ks, fQ, aq, auC, op, hR, at, it, B, bL, dA, qQ, aA, cz, asA, v, j, C, bX, q, J, aT, bBN, bq, ae, af, bA, aRR, VC, s, Y, asy, r, aMA, bQ, aDQ, bN, cl, acW, ac, alt, gX, mf, aDR, aoJ, dy, xb, bBM, iW, rr, ad, av, vJ, jo, eH, eZ, gl, aF, Fq, jg, dr, gk, OG, gZ, ei, asz, jr); #c1(GHR) c2(NP_001229329) c3(growth hormone receptor isoform 1 precursor) c4(84214, 84215, 84216) c5(84217, 84218, 84219, 84220, 84221, 84222, 84223, 84224, 84225, 84226, 84227, 84228, 84229, 84230, 84231, 84232) c6( )c7(T, pf, aoq, zs, e, al, atj, if, ci, ip, yo, aW, hl, LZ, g, eF, vA, ZT, bBU, OD, m, bBT, FG, fg, aj, JW, rc, adf, ec, dL, bl, avP, A, Rj, iw, ks, fQ, aq, auC, op, hR, at, it, B, bL, dA, qQ, aA, cz, asA, v, j, C, bX, q, J, aT, bBN, bq, ae, af, bA, aRR, VC, s, Y, asy, r, aMA, bQ, aDQ, bN, cl, acW, ac, alt, gX, mf, aDR, aoJ, dy, xb, bBM, iW, rr, ad, av, vJ, jo, eH, eZ, gl, aF, Fq, jg, dr, gk, OG, gZ, ei, asz, jr); #c1(GHR) c2(NP_001229389) c3(growth hormone receptor isoform 3 precursor) c4(84339) c5( )c6( )c7(T, pf, aoq, zs, e, al, atj, if, ci, ip, yo, aW, hl, LZ, g, eF, vA, ZT, bBU, OD, m, bBT, FG, fg, aj, JW, rc, adf, ec, dL, bl, avP, A, Rj, iw, ks, fQ, aq, auC, op, hR, at, it, B, bL, dA, qQ, aA, cz, asA, v, j, C, bX, q, J, aT, bBN, bq, ae, af, bA, aRR, VC, s, Y, asy, r, aMA, bQ, aDQ, bN, cl, acW, ac, alt, gX, mf, aDR, aoJ, dy, xb, bBM, iW, rr, ad, av, vJ, jo, eH, eZ, gl, aF, Fq, jg, dr, gk, OG, gZ, ei, asz, jr); #c1(GHR) c2(NP_001229391) c3(growth hormone receptor isoform 5 precursor) c4( )c5(84340, 84341, 84342, 84343, 84344, 84345) c6( )c7(T, pf, aoq, zs, e, al, atj, if, ci, ip, yo, aW, hl, LZ, g, eF, vA, ZT, bBU, OD, m, bBT, FG, fg, aj, JW, rc, adf, ec, dL, bl, avP, A, Rj, iw, ks, fQ, aq, auC, op, hR, at, it, B, bL, dA, qQ, aA, cz, asA, v, j, C, bX, q, J, aT, bBN, bq, ae, af, bA, aRR, VC, s, Y, asy, r, aMA, bQ, aDQ, bN, cl, acW, ac, alt, gX, mf, aDR, aoJ, dy, xb, bBM, iW, rr, ad, av, vJ, jo, eH, eZ, gl, aF, Fq, jg, dr, gk, OG, gZ, ei, asz, jr); #c1(GHR) c2(XP_011512333) c3(growth hormone receptor isoform X1) c4( )c5(302983) c6( )c7(T, pf, aoq, zs, e, al, atj, if, ci, ip, yo, aW, hl, LZ, g, eF, vA, ZT, bBU, OD, m, bBT, FG, fg, aj, JW, rc, adf, ec, dL, bl, avP, A, Rj, iw, ks, fQ, aq, auC, op, hR, at, it, B, bL, dA, qQ, aA, cz, asA, v, j, C, bX, q, J, aT, bBN, bq, ae, af, bA, aRR, VC, s, Y, asy, r, aMA, bQ, aDQ, bN, cl, acW, ac, alt, gX, mf, aDR, aoJ, dy, xb, bBM, iW, rr, ad, av, vJ, jo, eH, eZ, gl, aF, Fq, jg, dr, gk, OG, gZ, ei, asz, jr); #c1(GHRH) c2(XP_011527089) c3(somatoliberin isoform X1) c4(321550) c5(321551, 321552, 321553, 321554, 321555, 321556, 321557, 321558, 321559, 321560) c6( )c7(gh, T, bM, fM, aC, pY, OF, aA, adi, bBV, v, ci, fr, Tq, OH, aY, AD, JW, rc, ad, bBW, dP, ft, il, xW, pX, aql, oW, aT, bK, EL, ae, af, Yi, bA, yo, kl, d, s, aW, ff, Rj, af, en, cl, g, t, bz, avx, EJ, hq, pv, dR, OG, kP, eM, bo, uR, hL, hR, m, Ex); #c1(GHRH) c2(XP_011527090) c3(somatoliberin isoform X2) c4(321561) c5(321562, 321563, 321564, 321565, 321566, 321567) c6( ) c7(gh, T, bM, fM, aC, pY, OF, aA, adi, bBV, v, ci, fr, Tq, OH, aY, AO, JW, rc, ad, bBW, dP, ft, il, xW, pX, aql, oW, aT, bK, EL, ae, af, Yi, bA, yo, kl, d, s, aW, ff, Rj, aF, en, cl, g, t, bz, avx, EJ, hq, pv, dR, OG, kP, eM, bo, uR, hL, hR, m, Ex); #c1(GHRHR) c2(NP_000814) c3(growth hormone-releasing hormone receptor precursor) c4(16332) c5(16333, 16334, 16335, 16336, 16337, 16338, 16339) c6( )c7(T, dD, bBK, ea, lT, hy, bBS, eq, ci, asA, JW, ad, PC, dP, aT, iv, ae, af, bA, yo, BN, s, PE, Y, aW, Rj, vj, g, cl, eF, OG, kP, hR, gT, ne); #c1(GHRHR) c2(XP_011513565) c3(growth hormone-releasing hormone receptor isoform X1) c4(305850, 305851, 305852) c5(305853, 305854, 305855, 305856, 305857, 305858, 305859, 305860, 305861, 305862, 305863, 305864, 305865, 305866, 305867, 305868, 305869, 305870, 305871, 305872, 305873, 305874, 305875) c6( )c7(T, dD, bBK, ea, lT, hy, bBS, eq, ci, asA, JW, ad, PC, dP, aT, iv, ae, af, bA, yo, BN, s, PE, Y, aW, Rj, vj, g, cl, eF, OG, kP, hR, gT, ne); #c1(GHRL) c2(NP_001128416) c3(appetite-regulating hormone isoform 3) c4(49984) c5(49985, 49986, 49987, 49988, 49989, 49990, 49991, 49992) c6( )c7(bL, bM, T, dD, eR, fR, pf, aZ, vf, e, ht, qQ, al, aA, cz, tc, aj, v, j, ct, eq, aoj, wo, aY, OH, JW, cd, J, ad, rc, aw, av, hd, nv, iX, pX, Gb, aql, HC, aT, lG, jy, ji, K, qM, fb, ae, dl, Yi, bc, yo, kl, s, fc, r, kt, cu, aF, hp, g, iV, Z, ve, bJ, ar, rR, aD, ZT, mg, gZ, aqm, mf, OD, ei, y, X, iW); #c1(GHRL) c2(NP_001128417) c3(appetite-regulating hormone isoform 4) c4(49993) c5(49994, 49995, 49996, 49997, 49998, 49999, 50000, 50001) c6( )c7(bL, bM, T, dD, eR, fR, pf, aZ, vf, e, ht, qQ, al, aA, cz, tc, aj, v, j, ct, eq, aoj, wo, aY, OH, JW, cd, J, ad, rc, aw, av, hd, nv, iX, pX, Gb, aql, HC, aT, lG, jy, ji, K, qM, fb, ae, dl, Yi, bc, yo, kl, s, fc, r, kt, cu, aF, hp, g, iV, Z, ve, bJ, ar, rR, aD, ZT, mg, gZ, aqm, mf, OD, ei, y, X, iW); #c1(GHRL) c2(NP_001128418) c3(appetite-regulating hormone isoform 5) c4(50002) c5(50003, 50004, 50005, 50006, 50007, 50008, 50009, 50010) c6(50011) c7(bL, bM, T, dD, eR, fR, pf, aZ, vf, e, ht, qQ, al, aA, cz, tc, aj, v, j, ct, eq, aoj, wo, aY, OH, JW, cd, J, ad, rc, aw, av, hd, nv, iX, pX, Gb, aql, HC, aT, lG, jy, ji, K, qM, fb, ae, dl, Yi, bc, yo, kl, s, fc, r, kt, cu, aF, hp, g, iV, Z, ve, bJ, ar, rR, aD, ZT, mg, gZ, aqm, mf, OD, ei, y, X, iW); #c1(GHRL) c2(NP_001289752) c3(appetite-regulating hormone isoform 2 preproprotein) c4(114396) c5(114397, 114398, 114399, 114400, 114401, 114402, 114403, 114404, 114405, 114406, 114407, 114408, 114409, 114410, 114411, 114412, 114413, 114414, 114415, 114416, 114417, 114418, 114419, 114420, 114421, 114422, 114423, 114424, 114425, 114426, 114427, 114428, 114429, 114430, 114431, 114432, 114433, 114434, 114435, 114436, 114437, 114438, 114439, 114440, 114441, 114442, 114443, 114444, 114445, 114446, 114447, 114448, 114449, 114450, 114451, 114452, 114453, 114454, 114455, 114456, 114457, 114458, 114459, 114460, 114461, 114462, 114463, 114464, 114465, 114466, 114467, 114468, 114469, 114470, 114471, 114472, 114473, 114474, 114475, 114476, 114477, 114478, 114479, 114480, 114481, 114482, 114483, 114484, 114485, 114486, 114487, 114488, 114489, 114490, 114491, 114492, 114493, 114494, 114495, 114496, 114497, 114498, 114499, 114500, 114501, 114502, 114503, 114504, 114505, 114506, 114507, 114508, 114509, 114510, 114511, 114512, 114513, 114514, 114515, 114516, 114517, 114518, 114519, 114520, 114521, 114522, 114523, 114524, 114525, 114526, 114527, 114528, 114529, 114530, 114531, 114532, 114533, 114534, 114535, 114536, 114537, 114538, 114539, 114540, 114541, 114542, 114543, 114544, 114545, 114546, 114547, 114548, 114549, 114550, 114551, 114552, 114553, 114554, 114555, 114556, 114557, 114558, 114559) c6(114560) c7(bL, bM, T, dD, eR, fR, pf, aZ, vf, e, ht, qQ, al, aA, cz, tc, aj, v, j, ct, eq, aoj, wo, aY, OH, JW, cd, J, ad, rc, aw, av, hd, nv, iX, pX, Gb, aql, HC, aT, lG, jy, ji, K, qM, fb, ae, dl, Yi, bc, yo, kl, s, fc, r, kt, cu, aF, hp, g, iV, Z, ve, bJ, ar, rR, aD, ZT, mg, gZ, aqm, mf, OD, ei, y, X, iW); #c1(GHRL) c2(NP_001289754) c3(appetite-regulating hormone isoform 1 preproprotein) c4(114561, 114562, 114563) c5(114564, 114565, 114566, 114567, 114568, 114569, 114570, 114571, 114572, 114573, 114574, 114575, 114576, 114577, 114578, 114579, 114580, 114581, 114582, 114583, 114584, 114585, 114586, 114587, 114588, 114589, 114590, 114591, 114592, 114593, 114594, 114595, 114596) c6(114597) c7(bL, bM, T, dD, eR, fR, pf, aZ, vf, e, ht, qQ, al, aA, cz, tc, aj, v, j, ct, eq, aoj, wo, aY, OH, JW, cd, J, ad, rc, aw, av, hd, nv, iX, pX, Gb, aql, HC, aT, lG, jy, ji, K, qM, fb, ae, dl, Yi, bc, yo, kl, s, fc, r, kt, cu, aF, hp, g, iV, Z, ve, bJ, ar, rR, aD, ZT, mg, gZ, aqm, mf, OD, ei, y, X, iW); #c1(GHSR) c2(NP_004113) c3(growth hormone secretagogue receptor type 1 isoform 1b) c4(143676) c5(143677, 143678, 143679, 143680, 143681, 143682) c6( )c7(bL, bBM, T, dD, ea, aw, cz, bBX, tc, aj, v, j, afE, ci, eq, aoj, OH, cd, J, ad, rc, av, hd, iX, pX, aql, aT, iv, bj, ae, bA, yo, lR, s, aW, wf, r, fe, bu, cl, ve, iV, bz, OG, eM, dB, ZT, gX, hl, hR, mf, ei, bBY, y); #c1(GHSR) c2(NP_940799) c3(growth hormone secretagogue receptor type 1 isoform 1a) c4(259858) c5(259859, 259860, 259861, 259862, 259863, 259864) c6( )c7(bL, bBM, T, dD, ea, aw, cz, bBX, tc, aj, v, j, afE, ci, eq, aoj, OH, cd, J, ad, rc, av, hd, iX, pX, aql, aT, iv, bj, ae, bA, yo, lR, s, aW, wf, r, fe, bu, cl, ve, iV, bz, OG, eM, dB, ZT, gX, hL, hR, mf, ei, bBY, y); #c1(GID8) c2(NP_060366) c3(glucose-induced degradation protein 8 homolog) c4(195626, 195627) c5(195628, 195629, 195630, 195631, 195632, 195633, 195634, 195635, 195636, 195637, 195638, 195639) c6( )c7(aA); #c1(GIF) c2(NP_005133) c3(gastric intrinsic factor precursor) c4(153279, 153280) c5(153281, 153282, 153283, 153284, 153285, 153286, 153287, 153288, 153289, 153290, 153291, 153292) c6( )c7(bM, bzx, eR, Ke, aD, jx, mN, aj, ci, aY, mC, J, bCa, cd, ai, cc, F, aEE, rw, h, bBZ, dL, eL, ul, aZh, eZ, r, Ni, pR, ail, avG, bz, UP, dW, mz, my, aSu, fl, y, fh, kd, Ex, aMk); #c1(GIGYF1) c2(XP_005250589) c3(PERQ amino acid-rich with GYF domain-containing protein 1 isoform X1) c4(270416, 270417) c5(270418, 270419, 270420, 270421, 270422, 270423, 270424, 270425, 270426, 270427, 270428, 270429) c6( )c7(aW, cd, aT, oQ); #c1(GIGYF2) c2(NP_001096616) c3 (PERQ amino acid-rich with GYF domain-containing protein 2 isoform b) c4(41159, 41160, 41161, 41162, 41163) c5(41164, 41165, 41166, 41167, 41168, 41169, 41170, 41171, 41172, 41173, 41174, 41175, 41176, 41177, 41178, 41179, 41180, 41181, 41182, 41183, 41184, 41185) c6( )c7(aW, bCb, an, aT); #c1(GIGYF2) c2(NP_001096617) c3(PERO amino acid-rich with GYF domain-containing protein 2 isoform a) c4(41186, 41187) c5(41188, 41189, 41190, 41191, 41192, 41193, 41194, 41195, 41196, 41197, 41198, 41199, 41200, 41201, 41202, 41203, 41204, 41205, 41206, 41207) c6( ) c7(aW, bCb, an, aT); #c1(GIGYF2) c2(NP_001096618) c3(PERQ amino acid-rich with GYF domain-containing protein 2 isoform c) c4(41208, 41209, 41210, 41211, 41212, 41213) c5(41214, 41215, 41216, 41217, 41218, 41219, 41220, 41221, 41222, 41223, 41224, 41225, 41226, 41227, 41228, 41229, 41230, 41231, 41232, 41233, 41234, 41235, 41236, 41237, 41238, 41239, 41240, 41241, 41242, 41243, 41244, 41245, 41246, 41247, 41248, 41249, 41250, 41251, 41252, 41253, 41254, 41255) c6( )c7(aW, bCb, an, aT); #c1(GIMAP5) c2(NP_060854) c3(GTPase IMAP family member 5) c4( )c5(198124, 198125, 198126, 198127, 198128) c6( )c7(T, A, CM, eZ, aj, fL, y, J); #c1(GIMAP7) c2(NP_694968) c3(GTPase IMAP family member 7) c4(245506) c5(245507, 245508, 245509, 245510, 245511, 245512) c6( )c7(CM, A); #c1(GIMAP8) c2(XP_005250007) c3(GTPase IMAP family member 8 isoform X1) c4(269994) c5(269995, 269996, 269997, 269998, 269999, 270000) c6( )c7(ad); #c1(GINI) c2(NP_060146) c3(gypsy retrotransposon integrase-like protein 1) c4(194445, 194446) c5(194447, 194448, 194449, 194450, 194451, 194452, 194453, 194454, 194455, 194456, 194457, 194458) c6( )c7(y); #c1(GINS1) c2(NP_066545) c3(DNA replication complex GINS protein PSF1) c4(207319, 207320) c5(207321, 207322, 207323, 207324, 207325, 207326, 207327, 207328, 207329, 207330, 207331, 207332) c6( )c7(T, mR, bE, aW, if, aT, ey, K, qN); #c1(GINS2) c2(NP_057179) c3(DNA replication complex GINS protein PSF2) c4(189319) c5(189320, 189321, 189322, 189323, 189324, 189325) c6( )c7(T, os, mR, aW, qK, aT, oZ, hp); #c1(GIP) c2(NP_004114) c3(gastric inhibitory polypeptide preproprotein) c4(143683, 143684) c5(143685, 143686, 143687, 143688, 143689, 143690) c6( )c7(bL, dD, aoq, vf, ea, e, aj, Ja, dG, v, eq, J, mj, pX, jy, K, bq, H, fb, avH, s, vj, fc, hl, zR, iw, dq, Hu, g, mg, fP, cP, ei, gT, L); #c1(GIPC1) c2(NP_005707) c3(PDZ domain-containing protein GIPC1 isoform 1) c4(158673) c5(158674, 158675, 158676, 158677, 158678, 158679, 158680, 158681, 158682, 158683, 158684, 158685, 158686, 158687, 158688) c6( )c7(es, T, aC, eJ, aE, iA, aW, oV, aT, r, n, aj, m, t, ae, q, J, ad); #c1(GIPC1) c2(NP_974198) c3(PDZ domain-containing protein GIPC1 isoform 2) c4(262715) c5(262716, 262717, 262718, 262719, 262720, 262721, 262722, 262723, 262724, 262725) c6( )c7(es, T, aC, eJ, aE, iA, aW, oV, aT, r, n, aj, m, t, ae, q, J, ad); #c1(GIPC3) c2(NP_573568) c3(PDZ domain-containing protein GIPC3) c4(234552) c5(234553, 234554, 234555, 234556, 234557) c6( ) c7(cZ, bCd, bCc, alA, aT, dd, Bh); #c1(GIPC3) c2(XP_005259549) c3(PDZ domain-containing protein GIPC3 isoform X1) c4( )c5(276124, 276125) c6( )c7(cZ, bCd, bCc, alA, aT, dd, Bh); #c1(GIPR) c2(NP_000155) c3(gastric inhibitory polypeptide receptor precursor) c4(3562) c5(3563, 3564, 3565, 3566, 3567, 3568, 3569, 3570, 3571, 3572) c6( )c7(bL, tg, T, eM, vf, vj, pX, e, qQ, iw, iv, Ja, mf, dG, v, bq, ei, Hu, V, fq); #c1(GIT1) c2(NP_001078923) c3(ARF GTPase-activating protein GIT1 isoform 1) c4(38004, 38005) c5(38006, 38007, 38008, 38009, 38010, 38011) c6( )c7(qa, T, rR, cF, ot, ig, bE, aW, Y, if, r, aT, cd, bB, m, an, ae, q, ad); #c1(GIT1) c2(NP_054749) c3(ARF GTPase-activating protein GIT1 isoform 2) c4(177573) c5(177574, 177575, 177576, 177577, 177578, 177579, 177580, 177581, 177582, 177583, 177584, 177585) c6( )c7(qa, T, rR, cF, ot, ig, bE, aW, Y, if, r, aT, cd, bB, m, an, ae, q, ad); #c1(GIT1) c2(XP_011522986) c3(ARF GTPase-activating protein GIT1 isoform X1) c4( )c5(317461) c6( )c7(qa, T, rR, cF, ot, ig, bE, aW, Y, if, r, aT, cd, bB, m, an, ae, q, ad); #c1(GIT2) c2(NP_001128685) c3(ARF GTPase-activating protein GIT2 isoform 6) c4(50517, 50518) c5(50519, 50520, 50521, 50522, 50523, 50524, 50525, 50526, 50527, 50528, 50529, 50530) c6( )c7(aj, aoo, J, C, ig); #c1(GIT2) c2(NP_001128686) c3(ARF GTPase-activating protein GIT2 isoform 5) c4(50531, 50532) c5(50533, 50534, 50535, 50536, 50537, 50538, 50539, 50540, 50541, 50542, 50543, 50544) c6( ) c7(aj, ano, J, C, ig); #c1(GIT2) c2(NP_055591) c3(ARF GTPase-activating protein GIT2 isoform 3) c4(182152) c5(182153, 182154, 182155, 182156, 182157, 182158) c6( )c7(aj, aoo, J, C, ig); #c1(GIT2) c2(NP_476510) c3(ARF GTPase-activating protein GIT2 isoform 1) c4(230687) c5(230688, 230689, 230690, 230691, 230692, 230693) c6( )c7(aj, ano, J, C, ig); #c1(GIT2) c2(NP_476511) c3(ARF GTPase-activating protein GIT2 isoform 2) c4(230694, 230695, 230696) c5(230697, 230698, 230699, 230700, 230701, 230702, 230703, 230704, 230705, 230706, 230707, 230708, 230709, 230710, 230711, 230712, 230713, 230714) c6( )c7(aj, aoo, J, C, ig); #c1(GIT2) c2(NP_631940) c3(ARF GTPase-activating protein GIT2 isoform 4) c4(238363) c5(238364, 238365, 238366, 238367, 238368, 238369) c6( )c7(aj, aoo, J, C, ig); #c1(GIT2) c2(XP_006719772) c3(ARF GTPase-activating protein GIT2 isoform X4) c4(292966) c5(292967, 292968, 292969, 292970, 292971, 292972) c6( )c7(aj, ano, J, C, ig); #c1(GJA1) c2(NP_000156) c3(gap junction alpha-1 protein) c4(3573, 3574) c5(3575, 3576, 3577, 3578, 3579, 3580, 3581, 3582, 3583, 3584, 3585, 3586) c6( )c7(oO, T, bCf, ea, bB, Jd, ci, ip, biA, aE, Bc, ja, K, bCi, hu, brw, eL, Us, cp, aW, are, vs, g, as, pl, eF, bdW, eM, wK, ki, vA, alA, mx, PO, gn, asW, dD, wu, aw, ht, by, n, BH, qN, cd, bCj, bao, eW, bCh, acf, Bl, cc, wG, acA, h, SH, dL, Dz, bbG, ES, aq, kp, mo, dw, cM, op, bCe, dB, hk, wk, bD, B, bL, mr, wL, aA, cz, v, il, q, eA, Mg, aT, vH, cy, Nc, bk, bq, ae, eu, jf, s, oV, bCg, Y, fO, r, lo, en, t, bN, ac, wO, aJd, PN, im, bt, Bh, On, hl, bgQ, acl, ha, bS, eq, zU, tO, ad, dP, av, ib, od, sN, vc, wm, fb, vP, d, ff, eH, aF, EZ, avG, bz, arB, bE, Lj, dJ, gZ, aCP, lC); #c1(GJA3) c2(NP_068773) c3(gap junction alpha-3 protein) c4(210330) c5(210331, 210332, 210333, 210334, 210335, 210336) c6( )c7(b, kj, bP, bCk, aW, arm, aT, bee, i); #c1(GJA8) c2(XP_011507719) c3(gap junction alpha-& protein isoform X2) c4( )c5(297789, 297790, 297791, 297792, 297793, 297794, 297795, 297796, 297797, 297798, 297799, 297800, 297801) c6( )c7(bCl, T, dt, aWG, ha, fd, iD, ame, ci, q, ad, x, b, ib, aE, arm, aT, lQ, ae, De, kj, iU, aW, Y, ev, g, hf, t, cm, dr, sS, W, aPp, HN, aWy, m, od, i, bt, B); #c1(GJB1) c2(NP_000157) c3(gap junction beta-1 protein) c4(3587, 3588) c5(3589, 3590, 3591, 3592, 3593, 3594, 3595, 3596, 3597, 3598, 3599, 3600, 3601, 3602, 3603, 3604, 3605, 3606, 3607, 3608) c6( )c7(T, ob, jp, Pt, xF, bkY, agU, cV, aA, n, fr, ci, fu, bX, V, eA, ad, av, iu, b, no, aE, K, bU, amA, ae, dF, ls, s, mD, oV, bT, qS, bhi, aFT, bCn, aq, aF, g, t, cM, bz, dr, dR, es, ql, iA, bgy, kB, yW, Lj, mG, dh, bCo, bCm, bk, eo, i, afO, B, aof); #c1(GJB2) c2(NP_003995) c3(gap junction beta-2 protein) c4(142699) c5(142700, 142701, 142702, 142703, 142704, 142705, 142706, 142707, 142708, 142709) c6( )c7(T, aC, ci, adS, ip, aE, bCC, bU, hu, eL, Zm, bCu, aW, ih, uC, Jr, ajX, aGe, alA, bgi, cZ, bCB, wh, aw, ht, bCy, cV, by, n, asS, eW, b, aMT, dL, aQM, dd, bCq, aRa, aq, cM, ale, bCF, dB, hk, bbx, hL, awM, JM, it, B, arO, qc, dA, jp, bCp, aA, hy, Xr, oc, q, nX, aT, bq, bCv, rE, ae, bCA, le, ls, s, Y, r, en, t, f, Ci, bCE, mG, bCz, bCr, bvc, Hy, bCG, Bh, ao, sY, Dd, bCx, ad, aOZ, wD, av, bnC, lQ, KU, bCs, ff, xs, cr, ala, bCt, bz, bCD, pu, bCw, Lj, Km, i, rk); #c1(GJB6) c2(NP_00103691) c3(gap junction beta-6 protein) c4(41944) c5(41945, 41946, 41947, 41948, 41949, 41950, 41951, 41952, 41953, 41954, 41955, 41956, 41957, 41958, 41959, 41960, 41961, 41962) c6( )c7(cZ, Am, bCy, bCp, by, hy, bS, adS, oc, cd, asS, aDZ, bjA, nX, bCH, bCl, cy, SH, bCK, lQ, dd, bq, bmo, cB, bCu, bnY, xs, vs, bCt, bCJ, mG, alA, JM, awk, bgi, Bh, bca); #c1(GJC1) c2(NP_001073852) c3(gap junction gamma-1 protein) c4(36127) c5(36128, 36129, 36130, 36131, 36132, 36133, 36134, 36135, 36136, 36137) c6( )c7(ib, s, wK, aW, cz, r, aT, v, q); #c1(GJC2) c2(NP_065168) c3(gap junction gamma-2 protein) c4(204362) c5(204363, 204364, 204365, 204366, 204367, 204368) c6( )c7(bCM, KE, aV, lu, cb, aW, Pt, aT, Vv, bCL, PO, K, PN, bCN, mF, eo, V, bee, NG); #c1(GJC3) c2(NP_853516) c3(gap junction gamma-3 protein) c4( )c5(255143, 255144, 255145, 255146, 255147, 255148) c6( )c7(BM, Bh, mG, bX); #c1(GJC3) c2(XP_005250369) c3(gap junction gamma-3 protein isoform X1) c4(270283) c5( )c6( )c7(BM, Bh, mG, bX); #c1(GJD2) c2(NP_065711) c3(gap junction delta-2 protein) c4(205027) c5(205028, 205029, 205030, 205031, 205032, 205033) c6( )c7(bL, cZ, T, Be, iD, dd, Bd, bmo, ad); #c1(GJD3) c2(NP_689343) c3(gap junction delta-3 protein) c4(242657) c5(242658, 242659, 242660, 242661, 242662, 242663, 242664) c6( ) c7(s, BM, r, bq); #c1(GK) c2(NP_000158) c3(glycerol kinase isoform b) c4(3609) c5(3610, 3611, 3612, 3613, 3614, 3615) c6( )c7(bgp, bL, mO, xs, bCO, bzi, bgj, aj, uY, j, K, nA, v, aq, B, UF); #c1(GK) c2(NP_001121599) c3(glycerol kinase isoform c) c4(45825) c5(45826, 45827, 45828, 45829, 45830, 45831) c6( )c7(bgp, bL, mO, xs, bCO, bzi, bgj, aj, uY, j, K, nA, v, aq, B, UF); #c1(GK) c2(NP_001191948) c3(glycerol kinase isoform d) c4(81886, 81887) c5(81888, 81889, 81890, 81891, 81892, 81893) c6( )c7(bgp, bL, mO, xs, bCO, bzi, bgj, aj, uY, j, K, nA, v, aq, B, UF); #c1(GK) c2(NP_976325) c3(glycerol kinase isoform a) c4(263142, 263143) c5(263144, 263145, 263146, 263147, 263148, 263149, 263150, 263151, 263152, 263153, 263154, 263155) c6( )c7(bgp, bL, mO, xs, bCO, bzi, bgj, aj, uY, j, K, nA, v, aq, B, UF); #c1(GK) c2(XP_005274545) c3(glycerol kinase isoform X6) c4( )c5(285152, 285153, 285154, 285155, 285156, 285157) c6( )c7(bgp, bL, ml, xs, bCO, bzi, bgj, aj, uY, j, K, nA, v, aq, B, UF); #c1(GK) c2(XP_011543793) c3(glycerol kinase isoform X1) c4( )c5(342909) c6( )c7(bgp, bL, mO, xs, bCO, bzi, bgj, aj, uY, j, K, nA, v, aq, B, UF); #c1(GK) c2(XP_011543794) c3(glycerol kinase isoform X3) c4(342910) c5( )c6( )c7(bgp, bL, mO, xs, bCO, bzi, bgj, aj, uY, j, K, nA, v, aq, B, UF); #c1(GK) c2(XP_01543796) c3(glycerol kinase isoform X5) c4( )c5(342911, 342912, 342913, 342914, 342915) c6( )c7(bgp, bL, mO, xs, bCO, bzi, bgj, aj, uY, j, K, nA, v, aq, B, UF); #c1(GKN1) c2(NP_062563) c3(gastrokine-1) c4(201977) c5(201978, 201979, 201980, 201981, 201982, 201983) c6( )c7(T, s, fR, Kv, aW, if, az, aT, aA, aF, ci, qN, g, cm, nJ); #c1(GKN2) c2(NP_872342) c3(gastrokine-2 precursor) c4(256145) c5(256146, 256147, 256148, 256149, 256150, 256151, 256152, 256153) c6(256154, 256155) c7(av, T, aA, aF, g, ae, ad); #c1(GLA) c2(NP_000160) c3(alpha-galactosidase A precursor) c4(3616, 3617) c5(3618, 3619, 3620, 3621, 3622, 3623, 3624, 3625, 3626, 3627, 3628, 3629, 3630, 3631, 3632, 3633, 3634, 3635, 3636, 3637, 3638, 3639, 3640, 3641, 3642, 3643, 3644, 3645, 3646, 3647, 3648, 3649, 3650, 3651, 3652, 3653, 3654, 3655, 3656, 3657, 3658, 3659, 3660, 3661, 3662, 3663, 3664, 3665, 3666, 3667, 3668, 3669, 3670, 3671, 3672, 3673, 3674, 3675, 3676, 3677, 3678, 3679, 3680, 3681, 3682, 3683, 3684, 3685, 3686, 3687, 3688, 3689, 3690, 3691, 3692, 3693, 3694, 3695, 3696, 3697, 3698, 3699, 3700, 3701, 3702, 3703, 3704, 3705, 3706, 3707, 3708, 3709, 3710, 3711, 3712, 3713) c6(3714) c7(eR, bCQ, bCR, cz, cR, fo, aY, b, alu, cc, ds, M, vc, aa, bCP, asD, ih, dq, dw, dr, asE, mO, nM, ac, Lp, hO, Lq, dy, bdA, i); #c1(GLB1) c2(NP_000395) c3(beta-galactosidase isoform a preproprotein) c4(8459, 8460) c5(8461, 8462, 8463, 8464, 8465, 8466) c6( )c7(bM, T, aC, azf, fd, bB, lR, ci, x, xE, Al, dV, bCX, K, dQ, eL, bc, DA, akV, eG, aW, fc, La, g, pl, BE, eF, cm, lt, QA, bw, iA, bCT, iT, aYQ, U, cj, uo, k, Hb, m, afE, fr, cd, br, ai, bCZ, eW, ke, cc, cu, jy, h, hn, dL, bl, hQ, fe, aq, vU, iO, kT, bP, aYa, Lq, bD, B, bCS, bL, fM, aYP, bDa, fG, aA, cz, v, beZ, q, eA, bzg, XX, lg, nv, aMb, aT, cy, jj, aAt, rv, ae, bA, bCV, il, rh, oV, Y, bCU, r, lo, aYR, en, t, zz, pb, ck, nM, iM, bCY, hO, np, bt, hS, gx, kh, jh, btu, bS, ade, iJ, ad, hX, hd, bCW, bzk, aNp, ji, nJ, ul, DI, d, lx, je, ff, aF, Pd, iV, KM, bz, dr, qa, ek, mO, aMa, aAS, bE, XO, arx, cP, ra, lf, afO, y); #c1(GLB1) c2(NP_001129074) c3(beta-galactosidase isoform c preproprotein) c4(50738) c5(50739, 50740, 50741, 50742, 50743, 50744) c6( )c7(bM, T, aC, azf, fd, bB, lR, ci, x, xE, Al, dV, bCX, K, dl, el, bc, DA, akV, eG, aW, fc, La, g, pl, eE, eF, cm, Qt, QA, bw, iA, bCT, iT, aYQ, U, cj, uo, k, Hb, m, afE, fr, cd, br, ai, bCZ, eW, ke, cc, cU, jy, h, hn, dL, bl, hQ, fe, aq, vU, iO, KT, bP, aYa, Lq, bD, B, bCS, bL, fM, aYP, bDa, fG, aA, cz, v, beZ, q, eA, bzg, XX, lg, nv, aMb, aT, cy, jj, aAt, rv, ae, bA, bCV, iU, rh, oV, Y, bCU, r, lo, aYR, en, t, zz, pb, ck, nM, iM, bCY, hO, np, bt, hS, gx, kh, jh, btu, bS, ade, iJ, ad, hX, hd, bCW, bzk, aNp, ji, nJ, ul, DI, d, lx, je, ff, aF, Pd, iV, KM, bz, dr, qa, ek, mO, aMa, aAS, bE, XO, arx, cP, ra, lf, afO, y); #c1(GLCC11) c2(NP_612435) c3(glucocorticoid-induced transcript 1 protein) c4(236497) c5(236498, 236499, 236500, 236501, 236502, 236503, 236504) c6( ) c7(dw, bM, cm); #c1(GLCE) c2(XP_005254355) c3(D-glucuronyl C5-epimerase isoform X1) c4(273167, 273168) c5(273169, 273170, 273171, 273172, 273173, 273174, 273175, 273176, 273177, 273178, 273179, 273180) c6( )c7(ae, T, ft, aW, aT, bz, ad); #c1(GLDC) c2(NP_000161) c3(glycine dehydrogenase (decarboxylating), mitochondrial precursor) c4(3715) c5(3716, 3717, 3718, 3719, 3720, 3721) c6( )c7(T, eM, jh, dG, np, nA, bz, y, aZl, bAN, UZ, xt); #c1(GLDN) c2(NP_861454) c3(gliomedin) c4(255601, 255602) c5(255603, 255604, 255605, 255606, 255607, 255608) c6( )c7(ans, aq, T, B, yW); #c1(GLDN) c2(XP_011519803) c3(gliomedin isoform X1) c4( )c5(314550, 314551, 314552, 314553, 314554, 314555, 314556, 314557, 314558, 314559) c6( )c7(ans, aq, T, B, yW); #c1(GLE1) c2(NP_001003722) c3(nucleoporin GLE1 isoform 1) c4(20061, 20062) c5(20063, 20064, 20065, 20066, 20067, 20068, 20069, 20070, 20071, 20072, 20073, 20074) c6( )c7(bDb, bnq); #c1(GLE1) c2(NP_001490) c3(nucleoporin GLE1 isoform 2) c4(118164) c5(118165, 118166, 118167, 118168, 118169, 118170) c6( )c7(bDb, bnq); #c1(GLG1) c2(NP_001139138) c3(Golgi apparatus protein 1 isoform 2 precursor) c4(58772) c5(58773, 58774, 58775, 58776, 58777, 58778) c6( )c7(dB, T, aF, as, aT); #c1(GLG1) c2(NP_001139139) c3(Golgi apparatus protein 1 isoform 3 precursor) c4( )c5(58779, 58780, 58781, 58782, 58783, 58784) c6( )c7(dB, T, aF, as, aT); #c1(GLG1) c2(NP_036333) c3(Golgi apparatus protein 1 isoform 1 precursor) c4(173047, 173048) c5(173049, 173050, 173051, 173052, 173053, 173054) c6( )c7(dB, T, aF, as, aT); #c1(GLI1) c2(NP_001161081) c3(zinc finger protein GLI1 isoform 3) c4(65295) c5(65296, 65297, 65298, 65299, 65300, 65301, 65302, 65303, 65304, 65305) c6( )c7(T, aC, pf, if, fd, bB, axk, ci, bv, aE, hu, pk, eL, cp, aW, g, eF, cm, QA, Qt, zV, bDd, eM, cj, m, lG, ht, n, qN, ft, bDc, dL, hB, he, dx, pl, MD, fe, aq, kp, op, iO, dB, hk, eD, hL, zG, qF, YH, B, eR, fG, aA, bX, V, akW, q, eA, eJ, hW, afQ, aT, jj, ae, jf, s, Ar, nl, Y, r, en, cl, t, iK, sS, QG, kP, auo, nG, np, bt, hS, kh, id, acl, ad, hX, io, ji, lQ, fb, ul, d, qt, ff, eH, af, hp, aiz, iV, iR, bz, bE); #c1(GLI1) c2(XP_005268856) c3(zinc finger protein GLI1 isoform X2) c4( )c5(282122, 282123, 282124, 282125, 282126, 282127, 282128, 282129, 282130, 282131, 282132, 282133, 282134, 282135, 282136, 282137, 282138) c6( )c7(T, aC, pf, if, fd, bB, axk, ci, bv, aE, hu, pk, eL, cp, aW, g, eF, cm, QA, Qt, zV, bDd, eM, cj, m, lG, ht, n, qN, ft, bDc, dL, hB, he, dx, pl, MD, fe, aq, kp, op, iO, dB, hk, eD, hL, zG, qF, YH, B, eR, fG, aA, bX, V, akW, q, eA, eJ, hW, afQ, aT, jj, ae, jf, s, Ar, nO, Y, r, en, cl, t, ik, sS, QG, kP, auo, nG, np, bt, hS, kh, id, acl, ad, hX, io, ji, lO, fb, ul, d, qt, ff, eH, aF, hp, aiz, iV, iR, bz, bE); #c1(GLI1) c2(XP_011536491) c3(zinc finger protein GLI1 isoform X1) c4(333830, 333831, 333832, 333833) c5(333834, 333835, 333836, 333837, 333838, 333839, 333840, 333841, 333842, 333843, 333844, 333845, 333846, 333847) c6( ) c7(T, aC, pf, if, fd, bB, axk, ci, bv, aE, hu, pk, eL, cp, aW, g, eF, cm, DA, Qt, zV, bDd, eM, cj, m, lG, ht, n, qN, ft, bDc, dL, hB, he, dx, pl, MD, fe, aq, kp, op, iO, dB, hk, eD, hL, zG, qF, YH, B, eR, fG, aA, bX, V, akW, q, eA, eJ, hW, afd, aT, jj, ae, jf, s, Or, nO, Y, r, en, cl, t, ik, sS, QG, kP, auo, nG, np, bt, hS, kh, id, acl, ad, hX, io, ji, lQ, fb, uQ, d, qt, ff, eH, aF, hp, aiz, iV, iR, bz, bE); #c1(GLI1) c2(XP_011536492) c3(zinc finger protein GLI1 isoform X3) c4(333848, 333849) c5(333850, 333851, 333852, 333853, 333854, 333855, 333856) c6( )c7(T, aC, pf, if, fd, bB, axk, ci, bv, aE, hu, pk, eL, cp, aW, g, eF, cm, QA, Qt, zV, bDd, eM, cj, m, lG, ht, n, qN, ft, bDc, dL, hB, he, dx, pl, MD, fe, aq, kp, op, iO, dB, hk, eD, hL, zG, qF, YH, B, eR, fG, aA, bX, V, akW, q, eA, eJ, hW, afQ, aT, jj, ae, jf, s, Ar, nO, Y, r, en, cl, t, ik, sS, QG, kP, auo, nG, np, bt, hS, kh, id, acl, ad, hX, io, ji, lQ, fb, ul, d, qt, ff, eH, aF, hp, aiz, iV, iR, bz, bE); #c1(GLI2) c2(NP_005261) c3(zinc finger protein GLI2) c4(154415) c5(154416, 154417, 154418, 154419, 154420, 154421, 154422, 154423, 154424, 154425, 154426, 154427) c6( )c7(gh, T, kh, id, acz, lH, al, aA, bB, n, bBS, BF, bDe, Do, ad, biN, bv, HF, aE, arA, bOg, afQ, ld, aT, h, azm, dL, K, jj, ahK, fb, aOV, ae, af, eL, avP, bDf, B, qt, aW, Y, oV, eH, Az, ff, HL, kp, aF, en, eF, t, cm, hq, gk, sS, cA, bE, hk, zG, m, YH, ZW, X, hS); #c1(GLI2) c2(XP_006712485) c3(zinc finger protein GLI2 isoform X2) c4(286684, 286685) c5(286686, 286687, 286688, 286689, 286690, 286691, 286692, 286693, 286694, 286695, 286696) c6( ) c7(gh, T, kh, id, acz, lH, al, aA, bB, n, bBS, BF, bDe, Qo, ad, biN, bv, HF, aE, arA, bDg, afQ, ld, aT, h, azm, dL, K, jj, ahK, fb, aOV, ae, af, eL, avP, bDf, B, qt, aW, Y, oV, &H, Az, ff, HL, kp, af, en, eF, t, cm, hq, gk, sS, cA, bE, hk, zG, m, YH, ZW, X, hS); #c1(GLI2) c2(XP_011509275) c3 (zinc finger protein GLI2 isoform X5) c4(299898) c5(299899, 299900, 299901, 299902, 299903) c6( )c7(gh, T, kh, id, acz, lH, al, aA, bB, n, bBS, BF, bDe, Qo, ad, biN, bv, HF, aE, arA, bOg, afQ, ld, aT, h, azm, dL, K, jj, ahK, fb, aOV, ae, af, eL, avP, bDf, B, qt, aW, Y, oV, eH, Az, ff, HL, kp, aF, en, eF, t, cm, hq, gk, sS, cA, bE, hk, zG, m, YH, ZW, X, hS); #c1(GLI2) c2(XP_011509276) c3(zinc finger protein GLI2 isoform X6) c4( ) c5(299904) c6( )c7(gh, T, kh, id, acz, lH, al, aA, bB, n, bBS, BF, bDe, Do, ad, biN, bv, HF, aE, arA, bDg, afQ, ld, aT, h, azm, dL, K, jj, ahK, fb, aOV, ae, af, eL, avP, bDf, B, qt, aW, Y, oV, eH, Az, ff, HL, kp, aF, en, eF, t, cm, hq, gk, sS, cA, bE, hk, zG, m, YH, ZW, X, hS); #c1(GLI3) c2(XP_011513575) c3(transcriptional activator GLI3 isoform X1) c4(305884, 305885) c5(305886, 305887, 305888, 305889, 305890, 305891, 305892, 305893, 305894, 305895, 305896, 305897, 305898) c6( )c7(T, dA, aw, ha, atS, WT, aA, cz, bB, jx, aml, ci, PJ, zU, LI, akW, q, cd, ad, hX, eJ, XW, ahD, x, eW, aE, bDg, bDi, bxn, agi, afU, aT, h, BZ, cy, K, bDk, ZW, hH, avl, sj, jf, ak, anR, B, d, aW, aVQ, Az, CT, OS, r, fe, lo, bDI, g, as, fq, pb, aUv, atb, zV, bDj, ck, Di, sS, bDd, vW, c, iO, bE, lT, X, eB, U, zG, k, bkh, bC, art, bt, xg, bDh, bwD); #c1(GLIPR1) c2(NP_006842) c3(glioma pathogenesis-related protein 1 precursor) c4(168584) c5(168585, 168586, 168587, 168588, 168589, 168590) c6( )c7(T, d, dB, eL, dL, ey, hf, er, ae, eA, ad); #c1(GLIPR2) c2(NP_001273939) c3 (Golgi-associated plant pathogenesis-related protein 1 isoform b) c4(105926) c5(105927, 105928, 105929, 105930, 105931, 105932, 105933, 105934) c6(105935, 105936) c7(eA, oe, B, Hb); #c1(GLIPR2) c2(NP_071738) c3(Golgi-associated plant pathogenesis-related protein 1 isoform a) c4(211883) c5(211884, 211885, 211886, 211887, 211888, 211889, 211890, 211891, 211892) c6(211893, 211894) c7(eA, oe, B, Hb); #c1(GLIS2) c2(XP_005255699) c3(zinc finger protein GLIS2 isoform X1) c4(273810) c5(273811, 273812, 273813, 273814, 273815, 273816, 273817, 273818, 273819, 273820) c6( ) c7(ck, fl, T, Y, bDn, aq, vm, gm, dy, iY, bDm, Vz, aER, B, aY); #c1(GLIS3) c2(XP_006716794) c3 (zinc finger protein GLIS3 isoform X8) c4( ) c5(290464, 290465, 290466, 290467, 290468, 290469, 290470, 290471, 290472, 290473, 290474, 290475, 290476, 290477, 290478, 290479, 290480, 290481, 290482, 290483, 290484, 290485, 290486, 290487) c6( )c7(bL, A, mh, RN, mG, wB, aj, dy, K, Nj, aBZ, J, bDo); #c1(GLIS3) c2(XP_011516066) c3(zinc finger protein GLIS3 isoform X2) c4(309264, 309265) c5(309266, 309267, 309268, 309269, 309270, 309271, 309272) c6( )c7(bL, A, mh, RN, mG, wB, aj, dy, K, Nj, aBZ, J, bDo); #c1(GLIS3) c2(XP_011516068) c3(zinc finger protein GLIS3 isoform X1) c4(309273, 309274) c5(309275, 309276, 309277, 309278, 309279) c6( )c7(bL, A, mh, RN, mG, wB, aj, dy, K, Nj, aBZ, J, bDo); #c1(GLMN) c2(XP_011538848) c3(glomulin isoform X1) c4(336914) c5(336915, 336916, 336917, 336918, 336919, 336920) c6( ) c7(T, ob, aku, akw, asB, NT, bB, aks, ci, bDq, fu, Jt, q, alZ, aHF, h, H, mJ, BO, s, YC, r, ks, Kf, g, lm, t, bF, YI, GG, bP, bDr, akp, bE, afd, cj, bDp, aS, m, R, B); #c1(GLO1) c2(NP_006699) c3(lactoylglutathione lyase) c4(167235) c5(167236, 167237, 167238, 167239, 167240, 167241) c6( )c7(bL, T, dD, aC, eR, ea, eX, aA, bB, wo, aj, dG, ah, ct, j, eq, fr, lu, V, ww, eA, J, ad, KL, adX, aT, cy, eV, K, he, af, sj, A, aW, Y, aF, zC, gG, bN, hq, ck, rR, cA, adZ, kB, bE, iA, iT, U, aS, ei, it); #c1(GLOD4) c2(NP_057164) c3(glyoxalase domain-containing protein 4) c4(189240, 189241, 189242) c5(189243, 189244, 189245, 189246, 189247, 189248, 189249, 189250, 189251, 189252, 189253, 189254, 189255, 189256, 189257, 189258, 189259, 189260) c6( )c7(aq, B, ci); #c1(GLOD4) c2(XP_006721603) c3(glyoxalase domain-containing protein 4 isoform X1) c4(294111) c5(294112, 294113, 294114, 294115, 294116, 294117) c6( )c7(aq, B, ci); #c1(GLP1R) c2(NP_002053) c3(glucagon-like peptide 1 receptor precursor) c4(124091) c5(124092, 124093, 124094, 124095, 124096, 124097) c6( )c7(bL, aC, dD, ea, e, jN, cz, aj, dG, v, eq, J, mj, aE, pX, K, bq, uX, A, je, fc, ap, r, cm, iM, ac, U, gX); #c1(GLP2R) c2(NP_004237) c3(glucagon-like peptide 2 receptor) c4(144516, 144517, 144518) c5(144519, 144520, 144521, 144522, 144523, 144524, 144525, 144526, 144527, 144528, 144529, 144530, 144531, 144532, 144533, 144534, 144535, 144536, 144537, 144538, 144539, 144540, 144541, 144542) c6( )c7(bB, fb, bE, ji, iV); #c1 (GLP2R) c2(XP_011522379) c3(glucagon-like peptide 2 receptor isoform X1) c4(317063) c5( )c6( )c7(bB, fb, bE, ji, iV); #c1(GLRA1) c2(NP_000162) c3(glycine receptor subunit alpha-isoform 2 precursor) c4(3722, 3723) c5(3724, 3725, 3726, 3727, 3728, 3729, 3730, 3731, 3732) c6( )c7(ll, lF, aR, ft, aWm, ac, Js, aWN, HP, DK, aU, bDs, dg, Jo); #c1(GLRA1) c2(NP_001139512) c3(glycine receptor subunit alpha-1 isoform 1 precursor) c4(59455) c5(59456, 59457, 59458, 59459, 59460, 59461, 59462, 59463) c6( )c7(II, lF, aR, ft, aWm, ac, Js, aWN, HP, DK, aU, bDs, dg, Jo); #c1(GLRA1) c2(NP_001278929) c3(glycine receptor subunit alpha-1 isoform 3) c4( )c5(111051, 111052, 111053, 111054, 111055, 111056, 111057) c6( )c7(ll, lF, aR, ft, aWm, ac, Js, aWN, HP, DK, aU, bDs, dg, Jo); #c1(GLRA2) c2(NP_001112357) c3(glycine receptor subunit alpha-2 isoform A precursor) c4(43297, 43298) c5(43299, 43300, 43301, 43302, 43303, 43304) c6( )c7(ha, af); #c1(GLRA2) c2(NP_001112358) c3(glycine receptor subunit alpha-2 isoform B precursor) c4(43305) c5(43306, 43307, 43308, 43309, 43310, 43311) c6( )c7(ha, af); #c1(GLRA2) c2(NP_001165413) c3(glycine receptor subunit alpha-2 isoform C) c4( )c5(67662, 67663, 67664, 67665, 67666) c6( )c7(ha, af); #c1(GLRA2) c2(XP_006724550) c3(glycine receptor subunit alpha-2 isoform X1) c4(296068) c5(296069, 296070, 296071, 296072, 296073, 296074, 296075) c6( )c7(ha, af); #c1(GLRA3) c2(NP_001036008) c3(glycine receptor subunit alpha-3 isoform b precursor) c4(33678) c5(33679, 33680, 33681, 33682, 33683, 33684) c6( )c7(il, ha, Ea, b0); #c1(GLRA3) c2(NP_006520) c3(glycine receptor subunit alpha-3 isoform a precursor) c4(165961) c5(165962, 165963, 165964, 165965, 165966, 165967, 165968, 165969) c6( )c7(il, ha, Ea, b0); #c1(GLRB) c2(NP_001159532) c3(glycine receptor subunit beta isoform A precursor) c4(64265, 64266) c5(64267, 64268, 64269, 64270, 64271, 64272, 64273, 64274, 64275, 64276) c6( )c7(ll, lF, lu, aWN, bDs, nA, all); #c1(GLRB) c2(NP_001159533) c3(glycine receptor subunit beta isoform B precursor) c4( )c5(64277, 64278, 64279, 64280, 64281, 64282) c6( )c7(ll, lF, lu, aWN, bDs, nA, all); #c1(GLRX) c2(NP_001230588) c3(glutaredoxin-1) c4(86371) c5(86372, 86373, 86374, 86375, 86376, 86377, 86378, 86379, 86380, 86381, 86382, 86383, 86384, 86385, 86386, 86387, 86388, 86389, 86390, 86391, 86392, 86393) c6( )c7(T, jv, LC, Bc, oe, Ol, U, cz, aCl, v, vs, K, bq, ul, uC, aa); #c1(GLRX2) c2(NP_057150) c3(glutaredoxin 2 isoform 1) c4(189159) c5(189160, 189161, 189162, 189163, 189164, 189165) c6( )c7(gZ, cy, aw, ei, bu); #c1(GLRX2) c2(NP_932066) c3(glutaredoxin 2 isoform 2 precursor) c4(258779) c5(258780, 258781, 258782, 258783, 258784, 258785) c6( )c7(gZ, cy, aw, ei, bu); #c1(GLRX3) c2(NP_006532) c3(glutaredoxin-3) c4(166053) c5(166054, 166055, 166056, 166057, 166058, 166059, 166060, 166061, 166062, 166063, 166064, 166065, 166066, 166067) c6( )c7(es, T, ac, bE, aW, aT, cz, bB, ey, q, bz, B); #c1(GLRX5) c2(NP_057501) c3(glutaredoxin-related protein 5, mitochondrial precursor) c4(191185) c5(191186, 191187, 191188, 191189, 191190, 191191) c6( )c7(kA, oe, oj, oW, bDt, awK, avG, UZ, WT); #c1(GLS) c2(NP_001243239) c3(glutaminase kidney isoform, mitochondrial isoform 2) c4(90230) c5(90231, 90232, 90233, 90234, 90235, 90236) c6( )c7(T, dt, hV, ha, al, bB, axA, bS, ci, eA, x, av, mj, NS, aT, h, bj, dL, eL, Uf, d, aW, Y, aPf, aq, S, NR, g, eE, bz, dr, ck, gk, BU, qe, vD, eM, aiM, hR, pg, QF, k, ER, pw, y, bt, B); #c1(GLS) c2(NP_055720) c3(glutaminase kidney isoform, mitochondrial isoform 1 precursor) c4(183044, 183045) c5(183046, 183047, 183048, 183049, 183050, 183051, 183052, 183053, 183054, 183055, 183056, 183057) c6( )c7(T, dt, hV, ha, al, bB, axA, bS, ci, eA, x, av, mj, NS, aT, h, bj, dL, eL, Uf, d, aW, Y, aPf, aq, S, NR, g, eE, bz, dr, ck, gk, BU, qe, vD, eM, aiM, hR, pg, QF, k, ER, pw, y, bt, B); #c1(GLS2) c2(NP_037399) c3(glutaminase liver isoform, mitochondrial isoform 1 precursor) c4(175281, 175282) c5(175283, 175284, 175285, 175286, 175287, 175288, 175289, 175290, 175291, 175292, 175293, 175294) c6( )c7(iC, T, jv, d, bE, ev, ha, mg, bB, aq, Xj, mf, eA, as, il, y, Fc, B, Lg); #c1(GLTID1) c2(NP_653270) c3 (glycosyltransferase 1 domain-containing protein 1 precursor) c4(239439) c5(239440, 239441, 239442, 239443, 239444, 239445, 239446, 239447) c6(239448, 239449) c7(aw, ci, aT); #c1(GLTID1) c2(XP_005253610) c3(glycosyltransferase 1 domain-containing protein 1 isoform X2) c4(272581) c5(272582, 272583, 272584, 272585, 272586, 272587, 272588, 272589) c6(272590, 272591) c7(aw, ci, aT); #c1(GLTID1) c2(XP_011536258) c3(glycosyltransferase 1 domain-containing protein 1 isoform X1) c4(333594) c5(333595) c6( )c7(aw, ci, aT); #c1(GLTID1) c2(XP_011536259) c3(glycosyltransferase 1 domain-containing protein 1 isoform X3) c4( )c5(333596, 333597, 333598, 333599, 333600, 333601, 333602) c6(333603, 333604) c7(aw, ci, aT); #c1(GLT6D1) c2(XP_011516939) c3(glycosyltransferase 6 domain-containing protein 1 isoform X1) c4(310468) c5(310469, 310470, 310471, 310472, 310473, 310474) c6( )c7(uz, Tz); #c1(GLT8D1) c2(NP_001010983) c3(glycosyltransferase 8 domain-containing protein 1) c4(24395, 24396, 24397, 24398, 24399, 24400, 24401, 24402) c5(24403, 24404, 24405, 24406, 24407, 24408, 24409, 24410, 24411, 24412, 24413, 24414, 24415, 24416, 24417, 24418, 24419, 24420, 24421, 24422, 24423, 24424, 24425, 24426, 24427, 24428, 24429, 24430, 24431, 24432, 24433, 24434, 24435, 24436, 24437, 24438, 24439, 24440, 24441, 24442, 24443, 24444, 24445, 24446, 24447, 24448, 24449, 24450, 24451, 24452, 24453, 24454, 24455, 24456, 24457, 24458, 24459, 24460) c6( )c7(he, b0); #c1(GLTSCR1) c2(XP_005258890) c3 (glioma tumor suppressor candidate region gene 1 protein isoform X1) c4(275865) c5(275866, 275867, 275868, 275869, 275870, 275871) c6( )c7(dB, Fc, Ok, eA, ip); #c1(GLTSCR1) c2(XP_011525185) c3(glioma tumor suppressor candidate region gene 1 protein isoform X3) c4( )c5(319626, 319627, 319628) c6( ) c7(dB, Fc, Qk, eA, ip); #c1(GLTSCR2) c2(NP_056525) c3(glioma tumor suppressor candidate region gene 2 protein) c4(187917, 187918, 187919) c5(187920, 187921, 187922, 187923, 187924, 187925, 187926, 187927, 187928, 187929, 187930, 187931, 187932, 187933, 187934, 187935, 187936, 187937) c6( )c7(T, kl, d, dB, U, aT, aA, cm, aF, EY, g, as, eA, f, nJ, B, ad, Fc); #c1(GLUD1) c2(NP_005262) c3(glutamate dehydrogenase I, mitochondrial precursor) c4(154428, 154429, 154430, 154431, 154432, 154433) c5(154434, 154435, 154436, 154437, 154438, 154439, 154440, 154441, 154442, 154443, 154444, 154445, 154446, 154447, 154448, 154449, 154450, 154451, 154452, 154453, 154454, 154455, 154456, 154457, 154458) c6( )c7(yr, aYX, lV, dc, eu, ha, HN, aWJ, aip, bo, lG, aPf, jy, pw, il, xw, bDu, aR); #c1(GLUD1) c2(XP_011537971) c3(glutamate dehydrogenase (mitochondrial isoform X1) c4( )c5(335510, 335511, 335512, 335513, 335514, 335515, 335516, 335517, 335518, 335519, 335520) c6( )c7(yr, aYX, lV, dc, eu, ha, HN, aWJ, aip, bo, lG, aPf, jy, pw, il, xw, bDu, aR); #c1(GLUD2) c2(NP_036216) c3(glutamate dehydrogenase 2, mitochondrial precursor) c4(172425) c5(172426, 172427, 172428, 172429, 172430, 172431) c6( )c7(an); #c1(GLUL) c2(XP_006711341) c3(glutamine synthetase isoform X1) c4(285870, 285871) c5(285872, 285873, 285874, 285875, 285876, 285877, 285878, 285879, 285880, 285881, 285882, 285883, 285884, 285885, 285886, 285887, 285888, 285889, 285890, 285891, 285892, 285893, 285894, 285895) c6( )c7(bL, ka, jp, ha, aFc, bO, ti, aj, bS, v, ct, il, bfD, eA, aR, J, lF, av, bwo, h, bK, K, hK, DX, dF, af, A, d, B, kt, dq, aq, bDv, Z, Ym, bN, iO, Lj, ki, FB, SC, vy, nw, ra, bt, xg, gD); #c1(GLYAT) c2(NP_005829) c3(glycine N-acyltransferase isoform b) c4(159727) c5(159728, 159729, 159730, 159731, 159732, 159733) c6( )c7(bM, T, lP, if, bB, Vd, ci, qj, aJM, aE, dV, K, aW, CT, La, aoD, Ku, eF, g, eE, cm, aAW, qJ, eM, nB, m, n, nA, qN, eh, eW, ft, pe, cU, h, on, pi, dx, A, aq, aMJ, CO, ej, iO, ZL, lX, eD, QT, hR, aez, at, B, bL, rf, aA, j, Kh, AO, q, er, eA, oT, mj, hW, nv, aT, bK, lM, ae, jf, iF, s, aMG, NE, Y, r, en, cl, t, ik, hY, ck, BU, VJ, bDw, c, kB, nM, iM, jh, Hn, ad, hX, hd, ds, nJ, je, BO, qt, Fm, ff, eH, eZ, aF, bz, pu, bE, aS, be, ei, pC); #c1 (GLYAT) c2(NP_964011) c3(glycine N-acyltransferase isoform a) c4(262618) c5(262619, 262620, 262621, 262622, 262623, 262624, 262625, 262626, 262627, 262628) c6( ) c7(bM, T, lP, if, bB, Vd, ci, qj, aJM, aE, dV, K, aW, CT, La, aoD, Ku, eF, g, eE, cm, aAW, qJ, eM, nB, m, n, nA, qN, eh, eW, ft, pe, cU, h, on, pi, dx, A, aq, aMJ, CO, ej, iO, ZL, lX, eD, QT, hR, aez, at, B, bL, rf, aA, j, Kh, AO, q, er, eA, oT, mj, hW, nv, aT, bK, lM, ae, jf, iF, s, aMG, NE, Y, r, en, cl, t, iK, hY, ck, BU, VJ, bDw, c, kB, nM, iM, jh, Hn, ad, hX, hd, ds, nJ, je, BO, qt, Fm, ff, eH, eZ, af, bz, pu, bE, aS, be, ei, pC); #c1(GLYATL3) c2(NP_001010904) c3(glycine N-acyltransferase-like protein 3) c4(24036, 24037) c5(24038, 24039, 24040, 24041, 24042, 24043, 24044, 24045, 24046, 24047, 24048, 24049) c6( )c7(hb); #c1(GLYCTK) c2(NP_001138423) c3(glycerate kinase isoform 2) c4(56645) c5(56646, 56647, 56648, 56649, 56650, 56651, 56652, 56653, 56654, 56655, 56656, 56657, 56658, 56659) c6( )c7(bDy, bDx); #c1 (GLYCTK) c2(NP_660305) c3(glycerate kinase isoform 1) c4(240815, 240816, 240817, 240818, 240819, 240820) c5(240821, 240822, 240823, 240824, 240825, 240826, 240827, 240828, 240829, 240830, 240831, 240832, 240833, 240834, 240835, 240836, 240837, 240838, 240839, 240840, 240841, 240842, 240843, 240844, 240845, 240846, 240847, 240848, 240849, 240850, 240851, 240852) c6( )c7(bDy, bDx); #c1(GM2A) c2(NP_000396) c3(ganglioside GM2 activator isoform 1 precursor) c4(8467) c5(8468, 8469, 8470, 8471, 8472, 8473) c6( )c7(kf, bDa, bDz, lR, awM); #c1(GMCL1) c2(NP_848526) c3(germ cell-less protein-like 1) c4(253778) c5(253779, 253780, 253781, 253782, 253783, 253784) c6( )c7(cm, he, T, j, K); #c1(GMDS) c2(NP_001240775) c3(GDP-mannose 4.6 dehydratase isoform 2) c4(88801) c5(88802, 88803, 88804, 88805, 88806, 88807) c6( )c7(T, dH, bE, r, bB, q); #c1(GMDS) c2(NP_001491) c3(GDP-mannose 4.6 dehydratase isoform 1) c4(118171) c5(118172, 118173, 118174, 118175, 118176, 118177) c6( )c7(T, dH, bE, r, bB, q); #c1(GMFB) c2(NP_004115) c3(glia maturation factor beta) c4(143691, 143692) c5(143693, 143694, 143695, 143696, 143697, 143698, 143699, 143700, 143701, 143702, 143703, 143704) c6( )c7(cm, pd, d, bDA, bDB); #c1(GMFG) c2(NP_004868) c3(glia maturation factor gamma isoform 1) c4(150435) c5(150436, 150437, 150438, 150439, 150440, 150441, 15042, 150443, 150444, 150445) c6( )c7(t, e, j, m); #c1(GMIP) c2(NP_001275927) c3(GEM-interacting protein isoform 2) c4(107415) c5(107416, 107417, 107418, 107419, 107420, 107421) c6( ) c7(b); #c1(GMIP) c2(NP_057657) c3(GEM-interacting protein isoform 1) c4(192127) c5(192128, 192129, 192130, 192131, 192132, 192133) c6( )c7(bO); #c1(GML) c2(XP_011515271) c3(glycosyl-phosphatidylinositol-anchored molecule-like protein isoform X2) c4(308111) c5( ) c6( )c7(T, ht, eM, hu); #c1(GML) c2(XP_011515274) c3 (glycosyl-phosphatidylinositol-anchored molecule-like protein isoform X1) c4( ) c5(308112, 308113, 308114, 308115, 308116, 308117) c6( )c7(T, ht, eM, hu); #c1(GMNN) c2(XP_005249216) c3(geminin isoform X1) c4(269109, 269110, 269111, 269/12) c5(269113, 269114, 269115, 269116, 269117, 269118, 269119, 269120, 269121, 269122, 269123, 269124, 269125, 269126, 269127, 269128, 269129, 269130, 269131, 269132, 269133, 269134, 269135, 269136, 269137, 269138, 269139, 269140) c6( )c7(T, aC, dA, al, NT, jh, fd, uu, V, aE, dE, aT, h, pk, NE, aW, Y, aJD, nY, bz, gk, cj, eo, m, B); #c1(GMPPA) c2(NP_995319) c3(mannose-1-phosphate guanyltransferase alpha) c4(263807, 263808, 263809) c5(263810, 263811, 263812, 263813, 263814, 263815, 263816, 263817, 263818, 263819, 263820, 263821, 263822, 263823, 263824, 263825, 263826, 263827, 263828) c6( )c7(bDC); #c1(GMPPA) c2(XP_011509337) c3(mannose-1-phosphate guanyltransferase alpha isoform X5) c4( )c5(299907, 299908, 299909, 299910, 299911, 299912, 299913, 299914, 299915, 299916, 299917) c6( )c7(bDC); #c1(GMPPB) c2(NP_037466) c3(mannose-1-phosphate guanyltransferase beta isoform 1) c4(175650) c5(175651, 175652, 175653, 175654, 175655, 175656) c6( )c7(bDE, bDF, km, bDD); #c1(GMPPB) c2(NP_068806) c3(mannose-1-phosphate guanyltransferase beta isoform 2) c4(210650) c5(210651, 210652, 210653, 210654, 210655, 210656, 210657, 210658, 210659) c6( )c7(bDE, bDF, km, bDD); #c1(GMPR) c2(NP_006868) c3(GMP reductase 1) c4(168752) c5(168753, 168754, 168755, 168756, 168757, 168758) c6( )c7(Y, j); #c1(GMPR2) c2(NP_001002000) c3(GMP reductase 2 isoform 2) c4(19317) c5(19318, 19319, 19320, 19321, 19322, 19323, 19324, 19325, 19326, 19327, 19328, 19329, 19330, 19331, 19332, 19333) c6( )c7(ck, aW, aA, g, aT); #c1(GMPR2) c2(NP_001269951) c3(GMP reductase 2 isoform 4) c4(103286) c5(103287) c6( )c7(ck, aW, aA, g, aT); #c1(GMPR2) c2(NP_057660) c3(GMP reductase 2 isoform 1) c4(192154, 192155) c5(192156, 192157, 192158, 192159, 192160, 192161, 192162, 192163, 192164, 192165) c6( )c7(ck, aW, aA, g, aT); #c1(GMPR2) c2(XP_005267799) c3(GMP reductase 2 isoform X2) c4( ) c5(281247, 281248, 281249, 281250, 281251) c6( )c7(ck, aW, aA, g, aT); #c1(GMPS) c2(NP_003866) c3(GMP synthase [glutamine-hydrolyzing]) c4(141622) c5(141623, 141624, 141625, 141626, 141627, 141628, 141629, 141630, 141631, 141632, 141633, 141634, 141635, 141636, 141637, 141638) c6( )c7(bt, B, bq); #c1(GMPS) c2(XP_011511565) c3(GMP synthase [glutamine-hydrolyzing] isoform X1) c4(302301) c5( ) c6( )c7(bt, B, bq); #c1(GNA11) c2(NP_002058) c3(guanine nucleotide-binding protein subunit alpha-11) c4(124098) c5(124099, 124100, 124101, 124102, 124103, 124104, 124105, 124106) c6( )c7(bDG, T, aAl, Dy, TG, Y, aT, OM, bD, iP, HI, bDH, azh, avq); #c1 (GNA12) c2(NP_001269369) c3(guanine nucleotide-binding protein subunit alpha-12 isoform 2) c4(101310) c5(101311, 101312, 101313, 101314, 101315, 101316) c6( )c7(cZ, T, hl, Fj, pY, fG, eX, jh, cz, fd, j, ci, q, ad, hX, Do, pX, bDJ, aT, eV, dd, aW, ff, r, kt, en, iV, pL, t, bw, ig, dB, bDI, m, anx, y, bt, B, VB, On); #c1(GNA12) c2(NP_001280021) c3(guanine nucleotide-binding protein subunit alpha-12 isoform 4) c4(111272) c5(111273, 111274, 111275, 111276, 111277, 111278) c6( )c7(cZ, T, hl, Fj, pY, fG, eX, jh, cz, fd, j, ci, q, ad, hX, Do, pX, bDJ, aT, eV, dd, aW, ff, r, kt, en, iV, pl, t, bw, ig, dB, bDI, m, anx, y, bt, B, VB, On); #c1(GNA12) c2(NP_031379) c3(guanine nucleotide-binding protein subunit alpha-12 isoform 1) c4(172100) c5(172101, 172102, 172103, 172104, 172105, 172106) c6( )c7(cZ, T, hl, Fj, pY, fG, eX, jh, cz, fd, j, ci, q, ad, hX, Do, pX, bDJ, aT, eV, dd, aW, ff, r, kt, en, iV, pl, t, bw, ig, dB, bDI, m, anx, y, bt, B, VB, On); #c1(GNA13) c2(NP_001269354) c3(guanine nucleotide-binding protein subunit alpha-13 isoform 2) c4( )c5(101249, 101250, 101251, 101252, 101253, 101254) c6( )c7(aE, aW, Y, jh, aT, hp, pk, ad); #c1(GNA13) c2(NP_006563) c3(guanine nucleotide-binding protein subunit alpha-13 isoform 1) c4(166250, 166251, 166252) c5(166253, 166254, 166255, 166256, 166257, 166258, 166259, 166260, 166261, 166262, 166263, 166264) c6( )c7(aE, aW, Y, jh, aT, hp, pk, ad); #c1(GNA14) c2(NP_004288) c3(guanine nucleotide-binding protein subunit alpha-14) c4(144875) c5(144876, 144877, 144878, 144879, 144880, 144881) c6( )c7(il, cz, B, aA); #c1(GNA15) c2(NP_002059) c3(guanine nucleotide-binding protein subunit alpha-15) c4(124107) c5(124108, 124109, 124110, 124111, 124112, 124113) c6( )c7(x, ou, T, aE, pe, iW, hF, bt, cd, zn); #c1(GNAI1) c2(NP_001243343) c3(guanine nucleotide-binding protein G(i) subunit alpha-1 isoform 2) c4( )c5(90445, 90446, 90447, 90448, 90449) c6( )c7(H, fq, cd, dJ, L); #c1(GNAI1) c2(NP_002060) c3(guanine nucleotide-binding protein G(i) subunit alpha-isoform 1) c4(124114, 124115) c5(124116, 124117, 124118, 124119, 124/20, 124121, 124122) c6( )c7(H, fq, cd, dJ, L); #c1(GNA12) c2(NP_001159897) c3(guanine nucleotide-binding protein G(i) subunit alpha-2 isoform 2) c4(64950) c5(64951, 64952, 64953, 64954, 64955, 64956, 64957) c6( )c7(bL, oe, cz, bB, Ja, dG, ci, v, afv, zP, pX, bDK, beD, bDM, zQ, fc, hl, fe, Hu, bDL, bz, qa, bE, hR, OD, cP, ei, B); #c1(GNA12) c2(NP_001269546) c3(guanine nucleotide-binding protein G(i) subunit alpha-2 isoform 3) c4( )c5(101869, 101870, 101871, 101872, 101873, 101874) c6( )c7(bL, oe, cz, bB, Ja, dG, ci, v, afv, zP, pX, bDK, beD, bDM, zQ, fc, hl, fe, Hu, bDL, bz, qa, bE, hR, OD, cP, ei, B); #c1(GNA12) c2(NP_001269547) c3(guanine nucleotide-binding protein G(i) subunit alpha-2 isoform 4) c4( )c5(101875, 101876, 101877, 101878, 101879) c6( )c7(bL, oe, cz, bB, Ja, dG, ci, v, afv, zP, pX, bDK, beD, bDM, zQ, fc, hl, fe, Hu, bDL, bz, qa, bE, hR, OD, cP, ei, B); #c1(GNA12) c2(NP_001269548) c3(guanine nucleotide-binding protein G(i) subunit alpha-2 isoform 5) c4(101880) c5(101881, 101882, 101883, 101884, 101885, 101886) c6( )c7(bL, oe, cz, bB, Ja, dG, ci, v, afv, zP, pX, bDK, beD, bDM, zQ, fc, hl, fe, Hu, bDL, bz, qa, bE, hR, OD, cP, ei, B); #c1(GNAI2) c2(NP_001269549) c3(guanine nucleotide-binding protein G(i) subunit alpha-2 isoform 6) c4(101887) c5(101888, 101889, 101890, 101891, 101892, 101893) c6( )c7(bL, oe, cz, bB, Ja, dG, ci, v, afv, zP, pX, bDK, beD, bDM, zQ, fc, hl, fe, Hu, bDL, bz, qa, bE, hR, OD, cP, ei, B); #c1(GNAI2) c2(NP_002061) c3(guanine nucleotide-binding protein G(i) subunit alpha-2 isoform 1) c4(124123, 124124, 124125, 124126) c5(124127, 124128, 124129, 124130, 124131, 124132, 124133, 124134, 124135, 124136, 124137, 124138) c6( )c7(bL, oe, cz, bB, Ja, dG, ci, v, afv, zP, pX, bDK, beD, bDM, zQ, fc, hl, fe, Hu, bDL, bz, qa, bE, hR, OD, cP, ei, B); #c1(GNAL) c2(NP_001248373) c3(guanine nucleotide-binding protein G(olf) subunit alpha isoform 3) c4( )c5(93831, 93832, 93833, 93834, 93835) c6( )c7(aji, bDN, c, Wk, aU, WI, bK, Wm, bDO, aH); #c1(GNAL) c2(NP_892023) c3(guanine nucleotide-binding protein G(olf) subunit alpha isoform 1) c4(257632, 257633, 257634) c5(257635, 257636, 257637, 257638, 257639, 257640, 257641) c6( ) c7(aji, bDN, c, Wk, aU, WI, bK, Wm, bDO, aH); #c1(GNAL) c2(XP_011523956) c3(guanine nucleotide-binding protein G(olf) subunit alpha isoform X1) c4(318492) c5(318493, 318494, 318495, 318496, 318497, 318498, 318499, 318500, 318501, 318502, 318503, 318504, 318505, 318506, 318507, 318508, 318509, 318510, 318511, 318512) c6( )c7(aji, bDN, c, Wk, aU, WI, bK, Wm, bDO, aH); #c1(GNAD1) c2(NP_066268) c3(guanine nucleotide-binding protein G(o) subunit alpha isoform a) c4(206860) c5(206861, 206862, 206863, 206864, 206865, 206866) c6( )c7(hb, aW, vH, bDP, lm, B); #c1(GNAD1) c2(NP_620073) c3(guanine nucleotide-binding protein G(o) subunit alpha isoform b) c4(237269) c5(237270, 237271, 237272, 237273, 237274, 237275) c6( )c7(hb, aW, vH, bDP, lm, B); #c1(GNAQ) c2(NP_002063) c3(guanine nucleotide-binding protein G(q) subunit alpha) c4(124139, 124140, 124141) c5(124142, 124143, 124144, 124145, 124146, 124147, 124148, 124149, 124150, 124151, 124152, 124153, 124154, 124155, 124156, 124157, 124158, 124159) c6( )c7(T, aZ, hz, v, cd, aFy, vw, h, Dj, avq, cp, sj, Ck, Dg, kl, Y, Az, iP, Sc, bDR, Dy, aln, auw, qq, dX, bD, bDQ); #c1 (GNAS) c2(NP_000507) c3(protein GNAS isoform GNASL) c4(10685) c5(10686, 10687, 10688, 10689, 10690, 10691, 10692) c6( )c7(bDV, bM, T, aC, ahS, rV, vf, bB, Ja, ci, bEa, acX, pX, dV, K, EL, hu, qM, eL, bDT, eG, yo, Jl, aW, dg, g, eF, cm, bw, eM, nB, cj, uJ, OD, m, eg, OF, dD, lT, ht, aVF, bzb, n, aj, fr, nA, Ea, ml, JW, adf, jz, cc, Do, WB, Mv, RN, dL, bta, iw, ahJ, aq, Hu, cM, hk, bDX, bcd, hR, qq, oh, HB, bDS, oe, aA, cz, adi, hy, v, q, ahy, bDZ, nX, xW, yg, aT, ae, ne, jf, bDM, kl, s, NE, xR, Y, bEc, aXY, r, en, EJ, ck, bEb, bdn, QG, acW, ac, yl, aru, gT, aAO, ao, GI, Mb, bDU, id, pY, aZ, ml, auq, uu, eq, OH, bDY, ad, fU, py, aQS, lQ, bDW, yF, beD, aus, vj, ff, dq, anW, dr, qa, pv, es, OG, RV, bE, MC, X); #c1(GNAS) c2(NP_001070956) c3(protein GNAS isoform f) c4(35077) c5(35078, 35079, 35080, 35081, 35082, 35083, 35084) c6( )c7(bDV, bM, T, aC, ahS, rV, vf, bB, Ja, ci, bEa, acX, pX, dV, K, EL, hu, qM, eL, bDT, eG, yo, JI, aW, dg, g, eF, cm, bw, eM, nB, cj, uJ, OD, m, eg, OF, dD, lT, ht, aVF, bzb, n, aj, fr, nA, Ea, ml, JW, adf, jz, cc, Do, WB, Mv, RN, dL, bta, iw, ahJ, aq, Hu, cM, hk, bDX, bcd, hR, qq, oh, HB, bDS, oe, aA, cz, adi, hy, v, q, ahy, bDZ, nX, xW, yg, aT, ae, ne, jf, bDM, kl, s, NE, xR, Y, bEc, aXY, r, en, EJ, ck, bEb, bdn, QG, acW, ac, yl, aru, gT, aAD, ao, GI, Mb, bDU, id, pY, aZ, ml, auq, uu, eq, OH, bDY, ad, fU, py, aQS, lQ, bDW, yF, beD, aus, vj, ff, dq, anW, dr, qa, pv, es, OG, RV, bE, MC, X); #c1(GNAS) c2(NP_001070957) c3(protein GNAS isoform g) c4(35085) c5(35086, 35087, 35088, 35089, 35090, 35091, 35092) c6( )c7(bDV, bM, T, aC, ahS, rV, vf, bB, Ja, ci, bEa, acX, pX, dV, K, EL, hu, qM, eL, bDT, eG, yo, Jl, aW, dg, g, eF, cm, bw, eM, nB, cj, uJ, OD, m, eg, OF, dD, lT, ht, aVF, bzb, n, aj, fr, nA, Ea, ml, JW, adf, jz, cc, Do, WB, Mv, RN, dL, bta, iw, ahJ, aq, Hu, cM, hk, bDX, bcd, hR, qq, oh, HB, bDS, oe, aA, cz, adi, hy, v, q, ahy, bDZ, nX, xW, yg, aT, ae, ne, jf, bDM, kl, s, NE, xR, Y, bEc, aXY, r, en, EJ, ck, bEb, bdn, QG, acW, ac, yl, aru, gT, aAO, ao, GI, Mb, bDU, id, pY, aZ, ml, auq, uu, eq, OH, bDY, ad, fU, py, aQS, lQ, bDW, yF, beD, aus, vj, ff, dq, anW, dr, qa, pv, es, OG, RV, bE, MC, X); #c1(GNAS) c2(NP_057676) c3(protein NESP55 isoform NESP55) c4(192284, 192285, 192286) c5(192287, 192288, 192289, 192290, 192291, 192292, 192293, 192294, 192295, 192296, 192297, 192298, 192299, 192300, 192301, 192302, 192303, 192304, 192305, 192306, 192307, 192308, 192309) c6( )c7(bDV, bM, T, aC, ahS, rV, vf, bB, Ja, ci, bEa, acX, pX, dV, K, EL, hu, qM, eL, bDT, eG, yo, JI, aW, dg, g, eF, cm, bw, eM, nB, cj, uJ, OD, m, eg, OF, dD, lT, ht, aVF, bzb, n, aj, fr, nA, Ea, ml, JW, adf, jz, cc, Do, WB, Mv, RN, dl, bta, iw, ahJ, aq, Hu, cM, hk, bDX, bcd, hR, qq, oh, HB, bDS, oe, aA, cz, adi, hy, v, q, ahy, bDZ, nX, xW, yg, aT, ae, ne, jf, bDM, kl, s, NE, xR, Y, bEc, aXY, r, en, EJ, ck, bEb, bdn, QG, acW, ac, yl, aru, gT, aAO, ao, GI, Mb, bDU, id, pY, aZ, ml, auq, uu, eq, OH, bDY, ad, fU, py, aQS, lQ, bDW, yF, beD, aus, vj, ff, dq, anW, dr, qa, pv, es, OG, RV, bE, MC, X); #c1(GNAS) c2(NP_536350) c3(protein GNAS isoform XLas) c4(231263, 231264) c5(231265, 231266, 231267, 231268, 231269) c6( )c7(bDV, bM, T, aC, ahS, rV, vf, bB, Ja, ci, bEa, acX, pX, dV, K, EL, hu, qM, eL, bDT, eG, yo, JI, aW, dg, g, eF, cm, bw, eM, nB, cj, uJ, OD, m, eg, OF, dD, lT, ht, aVF, bzb, n, aj, fr, nA, Ea, ml, JW, adf, jz, cc, Do, WB, Mv, RN, dL, bta, iw, ahJ, aq, Hu, cM, hk, bDX, bcd, hR, qq, oh, HB, bDS, oe, aA, cz, adi, hy, v, q, ahy, bDZ, nX, xW, yg, aT, ae, ne, jf, bDM, kl, s, NE, xR, Y, bEc, aXY, r, en, EJ, ck, bEb, bdn, QG, acW, ac, yl, aru, gT, aAO, ao, GI, Mb, bDU, id, pY, aZ, ml, auq, uu, eq, OH, bDY, ad, fU, py, aQS, lQ, bDW, yF, beD, aus, vj, ff, dq, anW, dr, qa, pv, es, OG, RV, bE, MC, X); #c1 (GNAS) c2(NP_536351) c3(protein GNAS isoform GNASS) c4(231270) c5(231271, 231272, 231273, 231274, 231275, 231276, 231277, 231278, 231279, 231280) c6( )c7(bDV, bM, T, aC, ahS, rV, vf, bB, Ja, ci, bEa, acX, pX, dV, K, EL, hu, qM, eL, bDT, eG, yo, JI, aW, dg, g, eF, cm, bw, eM, nB, cj, uJ, OD, m, eg, OF, dD, lT, ht, aVF, bzb, n, aj, fr, nA, Ea, ml, JW, adf, jz, cc, Do, WB, Mv, RN, dL, bta, iw, ahJ, aq, Hu, CM, hk, bDX, bcd, hR, qq, oh, HB, bDS, oe, aA, cz, adi, hy, v, q, ahy, bDZ, nX, xW, yg, aT, ae, ne, jf, bDM, kl, s, NE, xR, Y, bEc, aXY, r, en, EJ, ck, bEb, bdn, QG, acW, ac, yl, aru, gT, aAD, ao, GI, Mb, bDU, id, pY, aZ, ml, auq, uu, eq, OH, bDY, ad, fU, py, aQS, lQ, bDW, yF, beD, aus, vj, ff, dq, anW, dr, qa, pv, es, OG, RV, bE, MC, X); #c1(GNAT1) c2(NP_653082) c3(guanine nucleotide-binding protein G(t) subunit alpha-1) c4(238840) c5(238841, 238842, 238843, 238844, 238845, 238846, 238847, 238848, 238849, 238850) c6( )c7(bEd, ayj, lR, bq); #c1(GNAT3) c2(NP_001095856) c3(guanine nucleotide-binding protein G(t) subunit alpha-3) c4(40863) c5(40864, 40865, 40866, 40867, 40868, 40869) c6( )c7(gU, om, ki, yc, v, bq); #c1(GNAZ) c2(NP_002064) c3(guanine nucleotide-binding protein G(z) subunit alpha) c4(124160, 124161) c5(124162, 124163, 124164, 124165, 124166, 124167, 124168, 124169, 124170, 124171, 124/72, 124/73) c6( )c7(Y, bK, yo); #c1(GNB1L) c2(NP_443730) c3(guanine nucleotide-binding protein subunit beta-like protein 1) c4(230048, 230049) c5(230050, 230051, 230052, 230053, 230054, 230055, 230056, 230057, 230058, 230059, 230060, 230061, 230062, 230063, 230064, 230065) c6( )c7(bO, gl, bK, he, agY, bN); #c1(GNB2L1) c2(NP_006089) c3(guanine nucleotide-binding protein subunit beta-2-like 1) c4(161817, 161818) c5(161819, 161820, 161821, 161822, 161823, 161824, 161825, 161826, 161827, 161828, 161829, 161830) c6( )c7(ID, T, aA, bB, n, aj, cd, ad, J, aT, K, ae, af, cp, ly, aW, Y, ff, aq, aF, en, g, f, ik, x, nM, bE, U, m, it, B, ao); #c1(GNB3) c2(NP_002066) c3(guanine nucleotide-binding protein G(I)/G(S)/G(T) subunit beta-3 isoform 1) c4(124/74) c5(124/75, 124/76, 124/77, 124178, 124/79, 124180) c6( )c7(fR, ea, e, al, bO, gf, aY, Vp, dY, ta, qM, Qe, tw, aW, Qv, uC, nB, Ji, vy, cj, cE, dD, Uc, aw, cl, aj, afE, ct, cd, TB, td, Mm, ec, wG, h, dl, ey, ux, A, cM, fq, bJ, KT, W, cA, tl, lB, AN, bL, eR, AL, mr, wl, De, jx, cz, cR, hz, hy, v, j, V, J, XX, uT, aT, bK, sR, ae, af, eu, kl, s, iU, sV, en, ik, bdn, pO, c, ac, cS, dy, sY, VR, akd, vr, oR, bS, oH, uu, eq, ad, nH, hd, db, oz, sj, d, CH, ff, EZ, Z, qa, gk, aED, gZ, aS, ei, tP, ra, ne); #c1(GNB4) c2(XP_006713784) c3(guanine nucleotide-binding protein subunit beta-4 isoform X1) c4(287348, 287349) c5(287350, 287351, 287352, 287353, 287354, 287355, 287356, 287357, 287358, 287359) c6( ) c7(el, bEe, dL, bU); #c1(GNB4) c2(XP_011511363) c3(guanine nucleotide-binding protein subunit beta-4 isoform X2) c4( )c5(301937, 301938, 301939, 301940, 301941, 301942) c6( )c7(el, bEe, dL, bU); #c1(GNB5) c2(NP_006569) c3(guanine nucleotide-binding protein subunit beta-5 isoform a) c4( )c5(166282, 166283, 166284, 166285, 166286, 166287, 166288, 166289, 166290, 166291, 166292, 166293) c6( )c7(bB, bE); #c1(GNB5) c2(NP_057278) c3(guanine nucleotide-binding protein subunit beta-5 isoform b) c4(189815, 189816, 189817, 189818) c5(189819, 189820, 189821, 189822, 189823, 189824) c6( )c7(bB, bE); #c1(GNB5) c2(XP_011519465) c3(guanine nucleotide-binding protein subunit beta-5 isoform X2) c4( )c5(314131, 314132, 314133, 314134, 314135, 314136) c6( )c7(bB, bE); #c1(GNE) c2(NP_001121699) c3(bifunctional UDP-N-acetylglucosamine 2-epimerase/N-acetylmannosamine kinase isoform 1) c4(46104, 46105) c5(46106, 46107, 46108, 46109, 46110, 46111, 46112, 46113, 46114) c6( )c7(T, ajy, fr, ad, no, aE, alp, Dq, Ws, oj, Ak, h, aiW, bl, ae, bht, Y, lb, alj, alm, bb, bEf, bi); #c1(GNE) c2(NP_001177312) c3(bifunctional UDP-N-acetylglucosamine 2-epimerase/N-acetylmannosamine kinase isoform 4) c4(72643) c5(72644, 72645, 72646, 72647, 72648, 72649, 72650, 72651, 72652, 72653, 72654) c6( )c7(T, ajy, fr, ad, no, aE, alp, Dq, Ws, oj, Ak, h, aiW, bl, ae, bht, Y, lb, alj, alm, bb, bEf, bi); #c1(GNE) c2(NP_001177317) c3(bifunctional UDP-N-acetylglucosamine 2-epimerase/N-acetylmannosamine kinase isoform 3) c4(72655, 72656) c5(72657, 72658, 72659, 72660, 72661, 72662) c6( )c7(T, ajy, fr, ad, no, aE, alp, Dq, Ws, oj, Ak, h, aiW, bl, ae, bht, Y, lb, alj, alm, bb, bEf, bi); #c1(GNE) c2(NP_005467) c3(bifunctional UDP-N-acetylglucosamine 2-epimerase/N-acetylmannosamine kinase isoform 2) c4( )c5(156419, 156420, 156421, 156422, 156423, 156424, 156425, 156426) c6( )c7(T, ajy, fr, ad, no, aE, alp, Dq, Ws, oj, Ak, h, aiW, bl, ae, bht, Y, lb, alj, alm, bb, bEf, bi); #c1(GNE) c2(XP_005251391) c3(bifunctional UDP-N-acetylglucosamine 2-epimerase/N-acetylmannosamine kinase isoform X1) c4(270935) c5(270936, 270937, 270938, 270939, 270940, 270941) c6( )c7(T, ajy, fr, ad, no, aE, alp, Dq, Ws, oj, Ak, h, aiW, bl, ae, bht, Y, lb, alj, alm, bb, bEf, bi); #c1(GNG10) c2(NP_001017998) c3(guanine nucleotide-binding protein G(I)/G(S)/G(0) subunit gamma-10 precursor) c4(26753) c5(26754, 26755, 26756, 26757, 26758, 26759) c6( )c7(Y); #c1(GNG11) c2(NP_004117) c3(guanine nucleotide-binding protein G(I)/G(S)/G(0) subunit gamma-ll precursor) c4(143705) c5(143706, 143707, 143708, 143709, 143710, 143711) c6( )c7(iu); #c1(GNG2) c2(XP_011535148) c3(guanine nucleotide-binding protein G(I)/G(S)/G(0) subunit gamma-2 isoform X1) c4(331985, 331986, 331987) c5(331988, 331989, 331990, 331991, 331992, 331993, 331994, 331995, 331996, 331997, 331998, 331999, 332000, 332001, 332002, 332003, 332004, 332005, 332006, 332007, 332008, 332009, 332010, 332011, 332012, 332013, 332014, 332015, 332016, 332017, 332018, 332019, 332020, 332021) c6( )c7(T, Y); #c1(GNG4) c2(NP_004476) c3(guanine nucleotide-binding protein G(I)/G(S)/G(0) subunit gamma-4 precursor) c4(146704) c5(146705, 146706, 146707, 146708, 146709, 146710, 146711, 146712, 146713, 146714, 146715, 146716, 146717, 146718, 146719, 146720, 146721, 146722) c6( )c7(fr, gv); #c1(GNG7) c2(NP_443079) c3(guanine nucleotide-binding protein G(I)/G(S)/G(0) subunit gamma-7 precursor) c4(229302) c5(229303, 229304, 229305, 229306, 229307) c6( )c7(ik, ht, gX); #c1(GNG8) c2(NP_150283) c3(guanine nucleotide-binding protein G(I)/G(S)/G(0) subunit gamma-8 precursor) c4(228042) c5(228043, 228044, 228045, 228046, 228047, 228048, 228049) c6(228050) c7(ou, xR, hl, eZ, cj, h, m, ci, y, bEg, uQ); #c1(GNG8) c2(XP_011525827) c3(guanine nucleotide-binding protein G(I)/G(S)/G(0) subunit gamma-8 isoform X2) c4( )c5(320235) c6( )c7(ou, xR, hl, eZ, cj, h, m, ci, y, bEg, uQ); #c1(GNGT1) c2(NP_068774) c3(guanine nucleotide-binding protein G(T) subunit gamma-Il precursor) c4(210337, 210338) c5(210339, 210340, 210341, 210342, 210343, 210344, 210345, 210346, 210347, 210348, 210349, 210350, 210351, 210352, 210353, 210354, 210355, 210356, 210357, 210358, 210359, 210360, 210361, 210362) c6( )c7(bM); #c1(GNGT2) c2(XP_006721882) c3(guanine nucleotide-binding protein G(I)/G(S)/G(0) subunit gamma-T2 isoform X1) c4(294338) c5(294339, 294340, 294341, 294342, 294343, 294344, 294345, 294346, 294347, 294348, 294349, 294350, 294351, 294352, 294353, 294354, 294355, 294356, 294357, 294358, 294359, 294360) c6( )c7(cj); #c1(GNL1) c2(NP_005266) c3(guanine nucleotide-binding protein-like 1) c4(154459, 154460, 154461, 154462, 154463, 154464, 154465) c5(154466, 154467, 154468, 154469, 154470, 154471, 154472, 154473, 154474, 154475, 154476, 154477, 154478, 154479, 154480, 154481, 154482, 154483, 154484, 154485, 154486, 154487, 154488, 154489, 154490, 154491, 154492, 154493, 154494, 154495, 154496, 154497, 154498, 154499, 154500, 154501, 154502, 154503, 154504, 154505, 154506, 154507, 154508, 154509, 154510, 154511, 154512, 154513, 154514, 154515, 154516, 154517, 154518, 154519, 154520, 154521, 154522, 154523, 154524, 154525, 154526, 154527, 154528, 154529, 154530, 154531, 154532, 154533, 154534, 154535, 154536, 154537, 154538, 154539, 154540, 154541, 154542, 154543, 154544, 154545, 154546, 154547, 154548, 154549, 154550, 154551, 154552, 154553, 154554, 154555, 154556, 154557, 154558, 154559, 154560, 154561, 154562, 154563, 154564, 154565, 154566, 154567, 154568, 154569, 154570, 154571, 154572, 154573, 154574, 154575, 154576, 154577, 154578, 154579, 154580, 154581, 154582, 154583, 154584, 154585, 154586, 154587, 154588, 154589, 154590, 154591, 154592, 154593, 154594, 154595, 154596, 154597, 154598, 154599, 154600, 154601, 154602, 154603, 154604, 154605) c6( )c7(hD, mR, wc, eZ, mP); #c1(GNL3) c2(NP_055181) c3(guanine nucleotide-binding protein-like 3 isoform 1) c4(179619, 179620, 179621) c5(179622, 179623, 179624, 179625, 179626, 179627, 179628, 179629, 179630, 179631, 179632, 179633, 179634, 179635, 179636) c6( )c7(NK, T, sw, jx, jh, aA, n, bS, ah, ct, ci, yO, qN, fr, eA, ad, lE, tT, ds, aT, cU, io, dL, bK, hu, hB, ae, eL, jf, d, iU, JI, aW, ff, eH, aq, aF, en, as, Z, eF, bz, ck, dB, AH, hh, bt, it, B, Ro); #c1(GNL3) c2(NP_996562) c3(guanine nucleotide-binding protein-like 3 isoform 2) c4(264316, 264317) c5(264318, 264319, 264320, 264321, 264322, 264323, 264324, 264325, 264326, 264327, 264328, 264329, 264330, 264331, 264332) c6( )c7(NK, T, sw, jx, jh, aA, n, bS, ah, ct, ci, yO, qN, fr, eA, ad, lE, tT, ds, aT, cU, io, dl, bk, hu, hB, ae, el, jf, d, iU, JI, aW, ff, eH, aq, aF, en, as, Z, eF, bz, ck, dB, AH, hh, bt, it, B, Ro); #c1(GNL3L) c2(NP_061940) c3(guanine nucleotide-binding protein-like 3-like protein) c4(201232) c5(201233, 201234, 201235, 201236, 201237, 201238, 201239, 201240, 201241, 201242) c6( ) c7(ae, ad, gX); #c1(GNLY) c2(NP_001289687) c3(granulysin isoform 1 precursor) c4(114360, 114361, 114362, 114363, 114364, 114365) c5(114366) c6( )c7(ck, bM, T, Dw, xS, aiV, qZ, bDJ, M, cj, h, fd, wR, Ds, pz, CQ, eg, f, bc); #c1(GNLY) c2(NP_006424) c3 (granulysin isoform 2 precursor) c4(164935, 164936) c5(164937, 164938, 164939, 164940, 164941, 164942, 164943, 164944, 164945, 164946, 164947, 164948, 164949, 164950, 164951, 164952, 164953, 164954, 164955, 164956, 164957, 164958, 164959, 164960, 164961, 164962, 164963, 164964 164965, 164966, 164967, 164968, 164969, 164970, 164971, 164972, 164973, 164974, 164975, 164976, 164977, 164978, 164979, 164980, 164981, 164982, 164983, 164984, 164985, 164986, 164987, 164988, 164989, 164990, 164991, 164992, 164993, 164994, 164995, 164996, 164997, 164998, 164999, 165000, 165001, 165002, 165003, 165004, 165005, 165006, 165007, 165008, 165009, 165010, 165011, 165012, 165013, 165014, 165015, 165016, 165017, 165018, 165019, 165020, 165021, 165022, 165023, 165024, 165025, 165026, 165027, 165028, 165029, 165030, 165031, 165032, 165033, 165034, 165035, 165036, 165037, 165038, 165039, 165040, 165041, 165042, 165043, 165044, 165045, 165046, 165047, 165048, 165049, 165050, 165051, 165052, 165053, 165054, 165055, 165056, 165057, 165058, 165059, 165060, 165061, 165062, 165063, 165064, 165065, 165066, 165067, 165068, 165069, 165070, 165071, 165072, 165073, 165074, 165075, 165076, 165077, 165078, 165079, 165080, 165081, 165082, 165083, 165084, 165085, 165086, 165087, 165088, 165089, 165090, 165091, 165092, 165093, 165094, 165095, 165096, 165097, 165098, 165099, 165100, 165101, 165102, 165103, 165104, 165105, 165106, 165107, 165108, 165109, 165110, 165111, 165112, 165113, 165114, 165115, 165116, 165117, 165118) c6( )c7(ck, bM, T, Dw, xS, aiV, qZ, bDJ, M, cj, h, fd, wR, Ds, pz, CO, eg, f, bc); #c1(GNLY) c2(NP_036615) c3(granulysin isoform 3) c4( )c5(174848, 174849, 174850, 174851, 174852, 174853, 174854, 174855, 174856, 174857, 174858, 174859, 174860, 174861, 174862, 174863, 174864, 174865, 174866, 174867, 174868, 174869, 174870, 174871, 174872, 174873, 174874, 174875, 174876, 174877, 174878, 174879, 174880, 174881, 174882, 174883, 174884, 174885, 174886, 174887, 174888, 174889, 174890, 174891, 174892, 174893, 174894, 174895, 174896, 174897, 174898, 174899, 174900, 174901, 174902, 174903, 174904, 174905, 174906, 174907, 174908, 174909, 174910, 174911, 174912, 174913, 174914, 174915, 174916, 174917, 174918, 174919, 174920, 174921, 174922, 174923, 174924, 174925, 174926, 174927, 174928, 174929, 174930, 174931, 174932, 174933, 174934, 174935, 174936, 174937, 174938, 174939, 174940, 174941, 174942) c6( )c7(ck, bM, T, Dw, xS, aiV, qZ, bDJ, M, cj, h, fd, wR, Ds, pz, CO, eg, f, bc); #c1 (GNLY) c2(XP_011530782) c3(granulysin isoform X4) c4(326811) c5( )c6( )c7(ck, bM, T, Dw, xS, aiV, qZ, bDJ, M, cj, h, fd, wR, Ds, pz, CQ, eg, f, bc); #c1 (GNLY) c2(XP_011530783) c3(granulysin isoform X5) c4( )c5(326812) c6( )c7(ck, bM, T, Dw, xS, aiV, qZ, bDJ, M, cj, h, fd, wR, Ds, pz, CQ, eg, f, bc); #c1(GNMT) c2(NP_061833) c3(glycine N-methyltransferase) c4(200688) c5(200689, 200690, 200691, 200692, 200693, 200694) c6( )c7(Na, T, jz, B, lp, aW, Y, wE, hl, aT, aq, Nc, iR, bt, ae, bEh, ad, vi); #c1(GNPAT) c2(NP_055051) c3(dihydroxyacetone phosphate acyltransferase) c4(178609, 178610) c5(178611, 178612, 178613, 178614, 178615, 178616, 178617, 178618, 178619, 178620, 178621, 178622) c6( )c7(mL, bEi, Fo, LR); #c1(GNPDA1) c2(XP_005268405) c3(glucosamine-6-phosphate isomerase 1 isoform X2) c4( ) c5(281804, 281805, 281806, 281807, 281808, 281809, 281810, 281811) c6( )c7(T, dt, dD, ea, ez, eq, ai, mC, hX, bEk, dV, OV, iU, iZ, bEj, eZ, aq, S, aFU, t, iK, bw, dr, yd, dW, oX, m, fl, ei, bt, fh, B); #c1(GNPDA1) c2(XP_011535839) c3(glucosamine-6-phosphate isomerase 1 isoform XI) c4(333064, 333065, 333066, 333067, 333068, 333069) c5(333070, 333071, 333072, 333073, 333074, 333075, 333076, 333077, 333078, 333079, 333080, 333081, 333082, 333083, 333084, 333085, 333086, 333087, 333088, 333089, 333090, 333091, 333092, 333093, 333094, 333095, 333096, 333097, 333098, 333099, 333100, 333101, 333102, 333103) c6( )c7(T, dt, dD, ea, ez, eq, ai, mC, hX, bEk, dV, OV, iU, iZ, bEj, eZ, aq, S, aFU, t, ik, bw, dr, yd, dW, oX, m, fl, ei, bt, fh, B); #c1(GNPDA2) c2(NP_001257809) c3(glucosamine-6-phosphate isomerase 2 isoform 2) c4(95651) c5(95652, 95653, 95654, 95655, 95656, 95657, 95658, 95659, 95660, 95661, 95662, 95663) c6( )c7(bL, bM, dY, hb, om, e, cz, Jd, v, fq); #c1(GNPDA2) c2(NP_612208) c3(glucosamine-6-phosphate isomerase 2 isoform 1) c4(236210, 236211) c5(236212, 236213, 236214, 236215, 236216, 236217, 236218, 236219, 236220, 236221, 236222, 236223) c6( )c7(bL, bM, dY, hb, om, e, cz, Jd, v, fq); #c1(GNPTAB) c2(NP_077288) c3(N-acetylglucosamine-1-phosphotransferase subunits alpha/beta precursor) c4(214955, 214956) c5(214957, 214958, 214959, 214960, 214961, 214962, 214963, 214964, 214965, 214966, 214967, 214968, 214969, 214970, 214971, 214972, 214973, 214974, 214975, 214976, 214977, 214978, 214979, 214980, 214981, 214982, 214983, 214984, 214985, 214986, 214987, 214988, 214989, 214990, 214991, 214992, 214993, 214994, 214995, 214996, 214997, 214998, 214999, 215000, 215001, 215002, 215003, 215004, 215005, 215006, 215007, 215008, 215009, 215010, 215011, 215012, 215013, 215014, 215015, 215016, 215017, 215018, 215019, 215020, 215021, 215022, 215023, 215024, 215025, 215026, 215027, 215028, 215029, 215030, 215031, 215032, 215033, 215034, 215035, 215036, 215037, 215038, 215039, 215040, 215041, 215042, 215043, 215044, 215045, 215046, 215047, 215048, 215049, 215050, 215051, 215052, 215053, 215054, 215055, 215056, 215057, 215058, 215059, 215060, 215061, 215062, 215063, 215064, 215065, 215066, 215067, 215068, 215069, 215070, 215071, 215072, 215073, 215074, 215075, 215076, 215077, 215078, 215079, 215080, 215081, 215082, 215083, 215084, 215085, 215086, 215087, 215088, 215089, 215090, 215091, 215092, 215093, 215094, 215095, 215096, 215097, 215098, 215099, 215100) c6( )c7(IE, bEm, aQQ, dq, bEl, Lq, beZ, bCS); #c1(GNPTAB) c2(XP_006719656) c3 (N-acetylglucosamine-1-phosphotransferase subunits alpha/beta isoform X2) c4(292868) c5(292869, 292870, 292871, 292872, 292873, 292874, 292875, 292876, 292877, 292878, 292879, 292880) c6( )c7(IE, bEm, aQQ, dq, bEl, Lq, beZ, bCS); #c1(GNPTG) c2(NP_115909) c3(N-acetylglucosamine-1-phosphotransferase subunit gamma precursor) c4(224626) c5(224627, 224628, 224629, 224630, 224631, 224632, 224633, 224634, 224635) c6( )c7(nC, bEm, beZ, Lq, bCS); #c1(GNRH1) c2(NP_000816) c3(progonadoliberin-1 isoform 1 precursor) c4( )c5(16340, 16341) c6( )c7(bEn, T, gU, bEp, cz, avO, ad, ip, UE, dP, aX, adX, aT, uV, Fg, ae, iz, jf, yo, kl, d, aW, Y, vj, Hu, aqP, cl, t, op, iP, adZ, dB, bEo, vA, hL, uz, m, UF, LS); #c1(GNRH1) c2(NP_001076580) c3(progonadoliberin-1 isoform 2 preproprotein) c4(37452) c5(37453, 37454, 37455, 37456, 37457, 37458, 37459, 37460) c6( )c7(bEn, T, gU, bEp, cz, avO, ad, ip, UE, dP, aX, adX, aT, uV, Fg, ae, iz, jf, yo, kl, d, aW, Y, vj, Hu, aqP, cl, t, op, iP, adZ, dB, bEo, vA, hl, uz, m, UF, LS); #c1(GNRH2) c2(NP_847901) c3(progonadoliberin-2 isoform c preproprotein) c4( )c5(253679, 253680, 253681, 253682, 253683, 253684, 253685, 253686, 253687) c6( )c7(T, op, vA, m, t, bEq, ae, ad); #c1(GNRH2) c2(NP_847902) c3 (progonadoliberin-2 isoform b preproprotein) c4(253688) c5(253689, 253690, 253691, 253692, 253693, 253694, 253695, 253696, 253697, 253698) c6( )c7(T, op, vA, m, t, bEq, ae, ad); #c1(GNRH2) c2(XP_011527522) c3(progonadoliberin-2 isoform X1) c4(322338) c5(322339) c6( )c7(T, op, vA, m, t, bEq, ae, ad); #c1 (GNRHR) c2(NP_000397) c3(gonadotropin-releasing hormone receptor isoform 1) c4(8474, 8475, 8476) c5(8477, 8478, 8479, 8480, 8481, 8482, 8483, 8484, 8485, 8486, 8487, 8488, 8489, 8490, 8491, 8492, 8493, 8494) c6( )c7(T, aZ, bBX, v, ci, ad, ip, UE, dP, yn, BJ, aT, Fg, ae, yo, kl, s, ND, aW, Y, aVQ, aeS, Hu, cl, t, bEr, aiv, Px, op, vA, hL, hR, m, UF, Th); #c1(GNRHR) c2(NP_001012781) c3(gonadotropin-releasing hormone receptor isoform 2) c4(25354) c5(25355, 25356, 25357, 25358, 25359, 25360) c6( )c7(T, aZ, bBX, v, ci, ad, ip, UE, dP, yn, BJ, aT, Fg, ae, yo, kl, s, ND, aW, Y, aVQ, aeS, Hu, cl, t, bEr, aiv, Px, op, vA, hl, hR, m, UF, Th); #c1 (GNS) c2(NP_002067) c3 (N-acetylglucosamine-6-sulfatase precursor) c4(124181, 124182, 124183) c5(124184, 124185, 124186, 124187, 124188, 124189, 124190, 124191, 124192, 124193, 124194, 124195, 124196, 124197, 124198, 124199, 124200, (24201) c6( )c7(IE, Lq, ey, kg); #c1(GOLGA1) c2(NP_002068) c3(Golgin subfamily A member 1) c4(124202) c5(124203, 124204, 124205, 124206, 124207, 124208, 124209, 124210, 124211, 124212, 124213, 124214) c6( )c7(bEs, Na, Nc); #c1(GOLGA1) c2(XP_006717126) c3(golgin subfamily A member 1 isoform X1) c4(290753, 290754) c5(290755, 290756, 290757, 290758, 290759, 290760) c6( )c7(bEs, Na, Nc); #c1(GOLGA2) c2(NP_004477) c3(Golgin subfamily A member 2) c4(146723, 146724, 146725) c5(146726, 146727, 146728, 146729, 146730, 146731) c6( )c7(T, bz); #c1(GOLGA2) c2(XP_005251987) c3(golgin subfamily A member 2 isoform X1) c4(271392) c5(271393, 271394, 271395, 271396, 271397, 271398, 271399, 271400) c6( )c7(T, bz); #c1(GOLGA2) c2(XP_005251989) c3(golgin subfamily A member 2 isoform X3) c4( )c5(271401, 271402, 271403, 271404, 271405, 271406, 271407, 271408, 271409, 271410) c6( )c7(T, bz); #c1(GOLGA3) c2(NP_001166028) c3(Golgin subfamily A member 3 isoform 2) c4(68071) c5(68072, 68073, 68074, 68075, 68076, 68077) c6( )c7(j); #c1(GOLGA3) c2(NP_005886) c3(Golgin subfamily A member 3 isoform 1) c4(160119, 160120) c5(160121, 160122, 160123, 160124, 160125, 160126, 160127, 160128, 160129, 160130, 160131, 160132, 160133, 160134, 160135, 160136) c6( )c7(j); #c1(GOLGA4) c2(NP_001166184) c3(Golgin subfamily A member 4 isoform 1) c4(68471) c5(68472, 68473, 68474, 68475, 68476, 68477) c6( )c7(aZJ, T, os, pc, il, aw, oZ, fr, lQ, Pk, ad); #c1(GOLGA4) c2(NP_002069) c3(Golgin subfamily A member 4 isoform 2) c4(124215) c5(124216, 124217, 124218, 124219, 124220, 124221) c6( )c7(aZJ, T, os, pc, iU, aw, oZ, fr, lQ, Pk, ad); #c1(GOLGA5) c2(NP_005104) c3(Golgin subfamily A member 5) c4(152905, 152906) c5(152907, 152908, 152909, 152910, 152911, 152912, 152913, 152914, 152915, 152916, 152917, 152918, 152919, 152920) c6( )c7(nM, hd, q, nB); #c1(GOLGA5) c2(XP_011535722) c3(golgin subfamily A member 5 isoform X1) c4(332961) c5(332962, 332963, 332964, 332965, 332966, 332967, 332968) c6( )c7(nM, hd, q, nB); #c1(GOLGA8B) c2(NP_001018861) c3(Golgin subfamily A member 8B) c4(27538, 27539) c5(27540, 27541, 27542, 27543, 27544, 27545, 27546, 27547, 27548, 27549, 27550, 27551, 27552, 27553, 27554, 27555) c6( )c7(Bd, q); #c1(GOLGA8B) c2(XP_011519900) c3(golgin subfamily A member 8B isoform X2) c4(314682) c5(314683, 314684, 314685, 314686, 314687, 314688) c6( )c7(Bd, q); #c1(GOLGB1) c2(NP_001243415) c3(Golgin subfamily B member 1 isoform 1) c4(90724) c5(90725, 90726, 90727, 90728, 90729, 90730, 90731, 90732, 90733, 90734, 90735) c6( )c7(jf, T, ac, h, Fw, cd); #c1(GOLGB1) c2(NP_004478) c3(Golgin subfamily B member 1 isoform 2) c4(146732) c5(146733, 146734, 146735, 146736, 146737, 146738) c6( )c7(jf, T, ac, h, Fw, cd); #c1 (GOLGB1) c2(XP_005247430) c3(golgin subfamily B member 1 isoform X6) c4(267833) c5( )c6( )c7(jf, T, ac, h, Fw, cd); #c1 (GOLGB1) c2(XP_006713653) c3(golgin subfamily B member 1 isoform X7) c4( )c5(287258, 287259, 287260, 287261, 287262, 287263) c6( )c7(jf, T, ac, h, Fw, cd); #c1 (GOLGB1) c2(XP_006713654) c3(golgin subfamily B member 1 isoform X8) c4(287264) c5(287265, 287266, 287267, 287268, 287269, 287270) c6( )c7(jf, T, ac, h, Fw, cd); #c1(GOLGB1) c2(XP_011511002) c3(golgin subfamily B member 1 isoform X9) c4(301458) c5(301459) c6( )c7(jf, T, ac, h, Fw, cd); #c1(GOLM1) c2(NP_808800) c3(Golgi membrane protein 1) c4(252912, 252913, 252914) c5(252915, 252916, 252917, 252918, 252919, 252920, 252921, 252922, 252923, 252924, 252925, 252926, 252927, 252928, 252929, 252930, 252931, 252932, 252933, 252934, 252935, 252936) c6( )c7(gk, T, jz, af, al, hl, jx, aA, it, aq, bEt, aF, bh, hE, ae, B, ad); #c1 (GOLPH3) c2(NP_071413) c3(Golgi phosphoprotein 3) c4(211530, 211531) c5(211532, 211533, 211534, 211535, 211536, 211537, 211538, 211539, 211540, 211541, 211542, 211543) c6( )c7(T, ht, jx, aA, n, oY, j, fr, q, ad, RD, eA, aT, cU, h, hu, MO, ae, d, arl, aW, ff, hl, r, aF, en, as, t, bz, bEu, Hp, cT, m, nz, it, B, afH); #c1(GOLT1A) c2(NP_940849) c3(vesicle transport protein GOT1A) c4(259939) c5(259940, 259941, 259942, 259943, 259944, 259945) c6( )c7(cz, br, bEv, bw, Yi, ek); #c1(GOLT1B) c2(NP_057156) c3(vesicle transport protein GOT1B) c4(189187) c5(189188, 189189, 189190, 189191, 189192, 189193) c6( )c7(wu, wm, br, bEv, bw, Yi, ek); #c1(GON4L) c2(NP_001269787) c3(GON-4-like protein isoform a) c4(102779) c5(102780, 102781, 102782, 102783, 102784, 102785, 102786, 102787, 102788, 102789) c6( ) c7(nA); #c1(GON4L) c2(NP_001269789) c3(GON-4-like protein isoform c) c4(102804, 102805) c5(102806, 102807, 102808, 102809, 102810, 102811, 102812, 102813, 102814, 102815, 102816, 102817) c6( )c7(nA); #c1(GON4L) c2(NP_115668) c3(GON-4-like protein isoform b) c4(223498) c5(223499, 223500, 223501, 223502, 223503, 223504) c6( )c7(nA); #c1(GON4L) c2(XP_011507960) c3(GON-4-like protein isoform X2) c4(298378) c5(298379, 298380, 298381, 298382, 298383, 298384) c6( )c7(nA); #c1(GOPC) c2(NP_001017408) c3(Golgi-associated PDZ and coiled-coil motif-containing protein isoform b) c4(26462) c5(26463, 26464, 26465, 26466, 26467, 26468, 26469, 26470) c6( )c7(T, ha, fd, aon, eA, b, aFl, aeO, aT, K, Tj, d, aeR, aW, Y, eH, CQ, eF, bz, bEw, Ew, aGY, cj, ER, i, lf); #c1(GOPC) c2(NP_065132) c3(Golgi-associated PDZ and coiled-coil motif-containing protein isoform a) c4(204114, 204115) c5(204116, 204117, 204118, 204119, 204120, 204121, 204122, 204123, 204124, 204125, 204/26, 204/27) c6( )c7(T, ha, fd, aon, eA, b, aFl, aeO, aT, K, Tj, d, aeR, aW, Y, eH, CQ, eF, bz, bEw, Ew, aGY, cj, ER, i, lf); #c1 (GORAB) c2(NP_001139511) c3(RAB6-interacting golgin isoform b precursor) c4( )c5(59449, 59450, 59451, 59452, 59453, 59454) c6( )c7(Uj, bEx, alc, uo, Un, B, bA); #c1(GORAB) c2(NP_689494) c3(RAB6-interacting golgin isoform a precursor) c4(242932, 242933) c5(242934, 242935, 242936, 242937, 242938, 242939) c6( )c7(Uj, bEx, alc, uo, Un, B, bA); #c1(GORASP1) c2(NP_114105) c3(Golgi reassembly-stacking protein 1 isoform 1) c4(222169) c5(222170, 222171, 222172, 222173, 222174, 222175) c6( )c7(oO, bM, T, os, if, al, bEy, eb, fd, lR, ci, bv, aE, K, eG, aW, az, asD, g, eE, cm, bw, axG, nB, cj, m, ht, aj, BF, qN, eW, kH, cU, pi, gi, fe, kp, dB, it, B, aA, C, V, q, J, eA, eJ, yg, aT, ae, af, aAi, jf, nO, Y, r, en, t, f, ik, pb, ck, iM, ac, c, ig, hr, bt, qb, oZ, dG, ad, XB, dP, hd, azq, ji, azR, gj, d, je, ff, xs, eZ, aF, iV, DF, bF, y, kS, X, brt); #c1(GORASP2) c2(NP_056345) c3(Golgi reassembly-stacking protein 2 isoform 1) c4(187172, 187173) c5(187174, 187175, 187176, 187177, 187178, 187179, 187180, 187181, 187182, 187183, 187184, 187185) c6( )c7(bL, v); #c1(GOSR1) c2(NP_001007026) c3(Golgi SNAP receptor complex member 1 isoform 2) c4(22389, 22390) c5(22391, 22392, 22393, 22394, 22395, 22396, 22397, 22398, 22399, 22400, 22401, 22402, 22403, 22404) c6( )c7(T, fM, iM, anT, je, aW, U, aT, Be, nw, hu, B, hX); #c1(GOSR1) c2(NP_004862) c3(Golgi SNAP receptor complex member 1 isoform 1) c4(150399, 150400) c5(150401, 150402, 150403, 150404, 150405, 150406, 150407, 150408, 150409, 150410, 150411, 150412) c6( )c7(T, fM, iM, anT, je, aW, U, aT, Be, nw, hu, B, hX); #c1(GOSR1) c2(XP_005258127) c3(Golgi SNAP receptor complex member 1 isoform X2) c4(275509) c5( )c6( )c7(T, fM, iM, anT, je, aW, U, aT, Be, nw, hu, B, hX); #c1(GOSR1) c2(XP_005258129) c3(Golgi SNAP receptor complex member 1 isoform X1) c4( )c5(275510, 275511, 275512, 275513, 275514, 275515, 275516, 275517) c6( )c7(T, fM, iM, anT, je, aW, U, aT, Be, nw, hu, B, hX); #c1(GOSR2) c2(NP_001012529) c3(Golgi SNAP receptor complex member 2 isoform C) c4(25059) c5(25060, 25061, 25062, 25063, 25064, 25065) c6( )c7(dD, bEA, ea, e, oe, cz, bEB, yZ, eq, yU, xw, bEz); #c1(GOSR2) c2(NP_004278) c3(Golgi SNAP receptor complex member 2 isoform A) c4(144778) c5(144779, 144780, 144781, 144782, 144783, 144784) c6( )c7(dD, bEA, ea, e, oe, cz, bEB, yZ, eq, yU, xw, bEz); #c1(GOSR2) c2(NP_473363) c3(Golgi SNAP receptor complex member 2 isoform B) c4(230396) c5(230397, 230398, 230399, 230400, 230401, 230402) c6( ) c7(dD, bEA, ea, e, oe, cz, bEB, yZ, eq, yU, xw, bEz); #c1(GOSR2) c2(XP_005257900) c3(Golgi SNAP receptor complex member 2 isoform X2) c4(275410) c5(275411, 275412, 275413, 275414, 275415, 275416) c6( )c7(dD, bEA, ea, e, oe, cz, bEB, yZ, eq, yU, xw, bEz); #c1(GOT1) c2(NP_002070) c3(aspartate aminotransferase, cytoplasmic) c4(124222, 124223, 124224) c5(124225, 124226, 124227, 124228, 124229, 124230, 124231, 124232, 124233, 124234, 124235, 124236, 124237, 124238, 124239, 124240, 124241, 124242) c6( )c7(iV, hB, bg, wc, bEC, fQ, io, mf, ek, br, bEv, ei, bw, bc, ae, cd, Yi, ad); #c1(GOT2) c2(NP_001273149) c3(aspartate aminotransferase, mitochondrial isoform 2 precursor) c4(104379) c5(104380, 104381, 104382, 104383, 104384, 104385, 104386) c6( )c7(bL, T, dD, ea, e, bB, aj, v, j, eq, q, J, eA, hd, aT, bci, bK, K, uV, kl, aW, fc, cr, r, g, qJ, nB, aep, ei); #c1(GOT2) c2(NP_002071) c3(aspartate aminotransferase, mitochondrial isoform 1 precursor) c4(124243, 124244) c5(124245, 124246, 124247, 124248, 124249, 124250, 124251, 124252, 124253, 124254, 124255) c6( )c7(bL, T, dD, ea, e, bB, aj, v, j, eq, q, J, eA, hd, aT, bci, bk, K, uV, kl, aW, fc, cr, r, g, qJ, nB, aep, ei); #c1(GP1BA) c2(NP_000164) c3 (platelet glycoprotein Ib alpha chain precursor) c4(3733, 3734) c5(3735, 3736, 3737, 3738, 3739, 3740, 3741, 3742, 3743, 3744, 3745, 3746, 3747) c6( )c7(sY, dD, uq, eR, qb, ea, aw, cz, vr, ez, aj, j, eq, bED, V, bEE, TB, ql, bpp, qh, rS, aT, iY, DU, K, DY, qk, eu, il, cH, ff, eZ, lo, vs, bEG, en, uC, cM, DI, ek, bEF, ac, wE, ag, aka, vb, qq, wx, aS, jl, vt, us); #c1(GP1BB) c2(NP_000398) c3 (platelet glycoprotein Ib beta chain precursor) c4(8495, 8496) c5(8497, 8498, 8499, 8500, 8501, 8502, 8503, 8504, 8505, 8506, 8507, 8508) c6( )c7(kl, RX, bpp, Az, DU, jQ, bD); #c1(GP2) c2(NP_001007241) c3(pancreatic secretory granule membrane major glycoprotein GP2 isoform 1 preproprotein) c4(22601) c5(22602, 22603, 22604, 22605, 22606, 22607) c6( )c7(A, boa); #c1(GP2) c2(NP_001007242) c3(pancreatic secretory granule membrane major glycoprotein GP2 isoform 3 precursor) c4( )c5(22608, 22609, 22610, 22611, 22612) c6( )c7(A, boa); #c1(GP2) c2(NP_001007243) c3(pancreatic secretory granule membrane major glycoprotein GP2 isoform 4 precursor) c4(22613) c5(22614, 22615, 22616, 22617, 22618) c6( )c7(A, boa); #c1(GP2) c2(NP_001493) c3(pancreatic secretory granule membrane major glycoprotein GP2 isoform 2 precursor) c4(118178) c5(118179, 118180, 118181, 118182, 118183, 118184) c6( )c7(A, boa); #c1(GP2) c2(XP_011544099) c3(pancreatic secretory granule membrane major glycoprotein GP2 isoform X1) c4(343267, 343268) c5(343269, 343270, 343271, 343272, 343273, 343274, 343275, 343276) c6( )c7(A, boa); #c1(GP5) c2(NP_004479) c3(platelet glycoprotein V precursor) c4(146739) c5(146740, 146741, 146742, 146743, 146744, 146745, 146746, 146747, 146748, 146749) c6( )c7(xu, vb, bpp, DU, xH, rH, jQ); #c1(GP6) c2(NP_001077368) c3(platelet glycoprotein VI isoform 1 precursor) c4(37759) c5(37760, 37761, 37762, 37763, 37764, 37765, 37766, 37767, 37768, 37769, 37770, 37771, 37772, 37773, 37774) c6( )c7(bL, dD, eR, pA, e, jx, aw, cz, vr, aj, j, Sv, eq, ai, cd, J, M, dz, qk, eu, hm, DL, bEH, wg, ac, vb, vy, dX, aS, dy, y); #c1(GPG) c2(NP_001242946) c3(platelet glycoprotein VI isoform 3 precursor) c4(89341) c5(89342, 89343, 89344, 89345, 89346, 89347, 89348, 89349, 89350, 89351, 89352, 89353, 89354, 89355, 89356, 89357, 89358, 89359, 89360, 89361, 89362, 89363, 89364, 89365, 89366, 89367, 89368, 89369, 89370) c6( )c7(bL, dD, eR, pA, e, jx, aw, cz, vr, aj, j, Sv, eq, ai, cd, J, M, dz, qk, eu, hm, DL, bEH, wg, ac, vb, vy, dX, aS, dy, y); #c1(GP6) c2(NP_057447) c3 (platelet glycoprotein VI isoform 2 precursor) c4(190930) c5(190931, 190932, 190933, 190934, 190935, 190936) c6( )c7(bL, dD, eR, pA, e, jx, aw, cz, vr, aj, j, Sv, eq, ai, cd, J, M, dz, qk, eu, hm, DL, bEH, wg, ac, vb, vy, dX, aS, dy, y); #c1(GP9) c2(NP_000165) c3(platelet glycoprotein IX precursor) c4(3748) c5(3749, 3750, 3751, 3752, 3753, 3754) c6( )c7(xu, ac, eR, bEl, eu, bpp, dx, DU, jQ, DY, bw, cM, sE, br); #c1(GPA33) c2(NP_005805) c3(cell surface A33 antigen precursor) c4(159588) c5(159589, 159590, 159591, 159592, 159593, 159594) c6( )c7(bB, bE, bA, cz); #c1(GPAA1) c2(NP_003792) c3(glycosylphosphatidylinositol anchor attachment 1 protein) c4(140880, 140881) c5(140882, 140883, 140884, 140885, 140886, 140887, 140888, 140889, 140890, 140891, 140892, 140893) c6( )c7(gk, T, aW, ff, al, aT, GC, dL, en, ik, eL, B, wT); #c1(GPALPP1) c2(NP_061029) c3(GPALPP motifs-containing protein 1) c4(199072) c5(199073, 199074, 199075, 199076, 199077, 199078, 199079, 199080, 199081, 199082) c6( )c7(bK, ac); #c1(GPAM) c2(XP_005270055) c3(glycerol-3-phosphate acyltransferase 1, mitochondrial isoform X1) c4(283063, 283064) c5(283065, 283066, 283067, 283068, 283069, 283070, 283071, 283072, 283073, 283074, 283075, 283076, 283077, 283078, 283079, 283080) c6( )c7(bL, Y, fc, v, ah, cP, bq, af); #c1(GPANK1) c2(NP_149417) c3(G patch domain and ankyrin repeat-containing protein 1) c4(227519, 227520, 227521, 227522) c5(227523, 227524, 227525, 227526, 227527, 227528, 227529, 227530, 227531, 227532, 227533, 227534, 227535, 227536, 227537, 227538, 227539, 227540, 227541, 227542, 227543, 227544, 227545, 227546, 227547, 227548, 227549, 227550, 227551, 227552, 227553, 227554, 227555, 227556, 227557, 227558, 227559, 227560, 227561, 227562, 227563, 227564, 227565, 227566, 227567, 227568, 227569, 227570, 227571, 227572, 227573, 227574, 227575, 227576, 227577, 227578, 227579, 227580, 227581, 227582, 227583, 227584, 227585, 227586, 227587, 227588, 227589, 227590, 227591, 227592, 227593, 227594, 227595, 227596, 227597, 227598, 227599, 227600, 227601, 227602, 227603, 227604, 227605, 227606, 227607, 227608, 227609, 227610, 227611, 227612, 227613, 227614, 227615, 227616, 227617, 227618, 227619, 227620, 227621, 227622, 227623, 227624, 227625, 227626, 227627, 227628, 227629, 227630, 227631, 227632, 227633, 227634, 227635, 227636, 227637, 227638, 227639, 227640, 227641, 227642, 227643, 227644, 227645, 227646, 227647, 227648, 227649, 227650, 227651, 227652) c6( )c7(y, iW, il, eZ, bX); #c1(GPANK1) c2(XP_006725801) c3(G patch domain and ankyrin repeat-containing protein 1 isoform X1) c4(296980, 296981, 296982) c5(296983, 296984, 296985, 296986, 296987, 296988, 296989, 296990, 296991, 296992, 296993, 296994, 296995, 296996, 296997, 296998, 296999, 297000, 297001, 297002, 297003, 297004) c6( )c7(y, iW, il, eZ, bX); #c1(GPANK1) c2(XP_011545613) c3(G patch domain and ankyrin repeat-containing protein 1 isoform XI) c4(344429, 344430, 344431, 344432, 344433, 344434) c5(344435, 344436, 344437, 344438, 344439, 344440, 344441, 344442, 344443, 344444, 344445, 344446, 344447, 344448, 344449, 344450, 344451, 344452, 344453, 344454, 344455, 344456, 344457, 344458, 344459, 344460, 344461, 344462, 344463, 344464, 344465, 344466, 344467, 344468, 344469, 344470, 344471, 344472, 344473, 344474, 344475, 344476, 344477, 344478, 344479, 344480, 344481, 344482, 344483, 344484, 344485, 344486, 344487, 344488, 344489, 344490, 344491, 344492, 344493, 344494, 344495, 344496, 344497, 344498, 344499, 344500, 344501, 344502, 344503, 344504, 344505, 344506, 344507, 344508, 344509, 344510, 344511, 344512, 344513, 344514, 344515, 344516, 344517, 344518, 344519, 344520, 344521, 344522, 344523, 344524, 344525, 344526, 344527, 344528, 344529, 344530, 344531, 344532, 344533, 344534, 344535, 344536, 344537, 344538, 344539, 344540, 344541, 344542, 344543, 344544, 344545, 344546, 344547, 344548, 344549, 344550, 344551) c6( )c7(y, iW, iQ, eZ, bX); #c1(GPAT2) c2(NP_997211) c3(glycerol-3-phosphate acyltransferase 2, mitochondrial) c4(265180, 265181) c5(265182, 265183, 265184, 265185, 265186, 265187, 265188, 265189, 265190, 265191) c6( )c7(aW, Y, aT); #c1(GPAT2) c2(XP_006712378) c3(glycerol-3-phosphate acyltransferase 2, mitochondrial isoform X11) c4(286616) c5(286617, 286618, 286619, 286620, 286621, 286622, 286623, 286624, 286625, 286626, 286627, 286628) c6( )c7(aW, Y, aT); #c1(GPATCH1) c2(NP_060495) c3(G patch domain-containing protein 1) c4(196231) c5(196232, 196233, 196234, 196235, 196236, 196237, 196238) c6( ) c7(bA); #c1(GPATCH2) c2(NP_001284683) c3(G patch domain-containing protein 2 isoform 2) c4(112302) c5(112303, 112304, 112305, 112306, 112307, 112308) c6( )c7(aW, T, cd, bu, aT); #c1(GPATCH2) c2(NP_060510) c3(G patch domain-containing protein 2 isoform 1) c4(196295) c5(196296, 196297, 196298, 196299, 196300, 196301) c6( )c7(aW, T, cd, bu, aT); #c1(GPATCH2L) c2(NP_060396) c3(G patch domain-containing protein 2-like isoform 1) c4(195805, 195806, 195807) c5(195808, 195809, 195810, 195811, 195812, 195813) c6( )c7(oe, k); #c1(GPATCH2L) c2(NP_060442) c3(G patch domain-containing protein 2-like isoform 2) c4(195961) c5(195962, 195963, 195964, 195965, 195966, 195967, 195968) c6( )c7(oe, k); #c1(GPATCH2L) c2(XP_011535237) c3(G patch domain-containing protein 2-like isoform X4) c4( )c5(332073, 332074, 332075, 332076, 332077, 332078, 332079, 332080, 332081, 332082, 332083, 332084, 332085, 332086, 332087) c6( )c7(oe, k); #c1(GPATCH8) c2(NP_001002909) c3(G patch domain-containing protein & isoform 1) c4(19728) c5(19729, 19730, 19731, 19732, 19733, 19734, 19735, 19736, 19737, 19738, 19739, 19740) c6( )c7(nH, zp); #c1(GPATCH8) c2(NP_001291869) c3(G patch domain-containing protein 8 isoform 3) c4(116484) c5(116485, 116486, 116487, 116488, 116489, 116490) c6( )c7(nH, zp); #c1(GPATCH8) c2(XP_011522859) c3(G patch domain-containing protein 8 isoform X2) c4(317341) c5(317342, 317343, 317344, 317345, 317346) c6( )c7(nH, zp); #c1(GPBAR1) c2(XP_011509046) c3(G-protein coupled bile acid receptor 1 isoform X1) c4(299647) c5(299648, 299649, 299650, 299651, 299652, 299653, 299654, 299655, 299656, 299657, 299658, 299659, 299660, 299661, 299662, 299663, 299664, 299665) c6( )c7(bL, bM, pf, aj, Ja, v, J, aE, pX, ju, h, Pn, he, Fg, d, Y, ff, ge, dq, hN, en, iV, hJ, bmj, Jh, jq, Ji, dX, ra, cE); #c1(GPC1) c2(NP_002072) c3(glypican-1 precursor) c4(124256) c5(124257, 124258, 124259, 124260, 124261, 124262) c6( )c7(T, aC, aE, EJ, af, aW, w, aal, fg, aT, uY, kp, K, Fq, eA, t, B, acM, L); #c1(GPC2) c2(NP_689955) c3(glypican-2 precursor) c4(244513) c5(244514, 244515, 244516, 244517, 244518, 244519, 244520, 244521) c6(244522, 244523) c7(T, aT); #c1(GPC3) c2(NP_001158089) c3(glypican-3 isoform 1 precursor) c4(63114) c5(63115, 63116, 63117, 63118, 63119, 63120) c6( )c7(T, kh, AS, Or, al, jx, ml, fg, n, ci, nA, bEJ, lu, er, hX, av, hd, ib, MS, jz, db, TK, qK, aT, io, Fx, AN, hB, pl, jf, s, aW, Y, ff, aq, aF, en, g, iR, t, cm, bz, Ld, qJ, iO, vW, nB, Qu, RW, ln, nG, m, KY, B, hS); #c1(GPC3) c2(NP_001158091) c3(glypican-3 isoform 4 precursor) c4(63121) c5(63122, 63123, 63124, 63125, 63126, 63127, 63128, 63129, 63130, 63131, 63132, 63133) c6( )c7(T, kh, AS, Or, al, jx, ml, fg, n, ci, nA, bEJ, lu, er, hX, av, hd, ib, MS, jz, db, TK, qK, aT, io, Fx, AN, hB, pl, jf, s, aW, Y, ff, aq, aF, en, g, iR, t, cm, bz, Ld, qJ, iO, vW, nB, Qu, RW, ln, nG, m, KY, B, hS); #c1(GPC3) c2(NP_004475) c3(glypican-3 isoform 2 precursor) c4(146696, 146697) c5(146698, 146699, 146700, 146701, 146702, 146703) c6( )c7(T, kh, AS, Or, al, jx, ml, fg, n, ci, nA, bEJ, lu, er, hX, av, hd, ib, MS, jz, db, TK, qK, aT, io, Fx, AN, hB, pl, jf, s, aW, Y, ff, aq, aF, en, g, iR, t, cm, bz, Ld, qJ, iD, vW, nB, Qu, RW, ln, nG, m, KY, B, hS); #c1(GPC4) c2(NP_001439) c3(glypican-4 precursor) c4(117833, 117834, 117835) c5(117836, 117837, 117838, 117839, 117840, 117841, 117842, 117843, 117844, 117845, 117846, 117847, 117848, 117849, 117850, 117851, 117852, 117853) c6( )c7(TY, Az, fg, rO, fq, er); #c1(GPC5) c2(NP_004457) c3(glypican-5 precursor) c4(146464) c5(146465, 146466, 146467, 146468, 146469, 146470) c6( )c7(bv, av, ac, eM, lp, jh, rf, gZ, Da, j, ah, V, bz, cM, fq, iu, sj); #c1(GPC6) c2(NP_005699) c3(glypican-6 precursor) c4(158633) c5(158634, 158635, 158636, 158637, 158638, 158639) c6( )c7(agJ, pH, Kc, aHe, bP, pN, aW, oe, Az, aT, r, v, ci, V, q, atb); #c1(GPCPD1) c2(NP_062539) c3(glycerophosphocholine phosphodiesterase GPCPD1) c4(201867) c5(201868, 201869, 201870, 201871, 201872, 201873) c6( )c7(bl); #c1(GPCPD1) c2(XP_005260818) c3 (glycerophosphocholine phosphodiesterase GPCPD1 isoform X2) c4(276910, 276911) c5(276912, 276913, 276914, 276915, 276916, 276917, 276918, 276919, 276920, 276921, 276922) c6( )c7(bl); #c1(GPCPD1) c2(XP_011527609) c3(glycerophosphocholine phosphodiesterase GPCPD1 isoform X3) c4( )c5(322492) c6( )c7(bl); #c1(GPD1) c2(NP_001244128) c3(glycerol-3-phosphate dehydrogenase [NAD(+)], cytoplasmic isoform 2) c4( )c5(91974, 91975, 91976, 91977, 91978, 91979) c6( )c7(bL, aW, bEK, B, aT); #c1(GPD1) c2(NP_005267) c3(glycerol-3-phosphate dehydrogenase [NAD(+)], cytoplasmic isoform 1) c4(154606, 154607) c5(154608, 154609, 154610, 154611, 154612, 154613) c6( )c7(bL, aW, bEK, B, aT); #c1(GPD1L) c2(NP_055956) c3(glycerol-3-phosphate dehydrogenase 1-like protein) c4(184686) c5(184687, 184688, 184689, 184690, 184691, 184692) c6( )c7(rf, bEL, n, ajM, j, EZ, ik, cd); #c1(GPD2) c2(NP_001076581) c3(glycerol-3-phosphate dehydrogenase, mitochondrial precursor) c4(37461, 37462, 37463, 37464) c5(37465, 37466, 37467, 37468, 37469, 37470, 37471, 37472, 37473, 37474, 37475, 37476, 37477, 37478, 37479, 37480, 37481, 37482, 37483, 37484, 37485, 37486, 37487, 37488, 37489, 37490, 37491, 37492, 37493, 37494, 37495, 37496, 37497, 37498, 37499, 37500) c6( )c7(bL, aHq, bEN, dY, bEM, K, nA, bq, ais, jw, ad, bA); #c1(GPD2) c2(XP_011509282) c3(glycerol-3-phosphate dehydrogenase, mitochondrial isoform X4) c4( )c5(299905) c6( )c7(bL, aHq, bEN, dY, bEM, K, nA, bq, ais, jw, ad, bA); #c1(GPER1) c2(NP_001091671) c3(G-protein coupled estrogen receptor 1) c4(38602, 38603, 38604) c5(38605, 38606, 38607, 38608, 38609, 38610, 38611, 38612, 38613, 38614, 38615, 38616, 38617, 38618, 38619, 38620, 38621, 38622, 38623, 38624, 38625, 38626, 38627, 38628, 38629, 38630, 38631, 38632, 38633, 38634) c6( )c7(gh, T, dD, ea, e, atS, aA, hy, j, ci, eq, AD, V, ad, dP, aT, cy, hP, ae, d, BN, zH, aW, wc, aF, cl, t, op, kP, vA, hL, gZ, m, ei, ne); #c1(GPHA2) c2(NP_570125) c3 (glycoprotein hormone alpha-2 precursor) c4( )c5(233990, 233991, 233992, 233993, 233994, 233995, 233996, 233997, 233998, 233999, 234000, 234001, 234002) c6(234003, 234004, 234005, 234006) c7(Y); #c1(GPHA2) c2(XP_011543076) c3(glycoprotein hormone alpha-2 isoform X2) c4(341876) c5(341877) c6( )c7(Y); #c1(GPHN) c2(NP_001019389) c3(gephyrin isoform 2) c4(27748, 27749) c5(27750, 27751, 27752, 27753, 27754, 27755, 27756, 27757, 27758, 27759, 27760, 27761) c6( )c7(ll, ck, lF, Cq, bEO, s, aWN, iZ, oe, ha, nf, cu, K, il, af, cv); #c1(GPHN) c2(NP_065857) c3(gephyrin isoform 1) c4(205926) c5(205927, 205928, 205929, 205930, 205931, 205932) c6( )c7(ll, ck, lF, Cq, bEO, s, aWN, iZ, oe, ha, nf, cu, K, il, af, cv); #c1(GPI) c2(NP_001171651) c3(glucose-6-phosphate isomerase isoform 1) c4(71154) c5(71155, 71156, 71157, 71158, 71159, 71160) c6( )c7(ID, eW, T, dt, dD, qo, aC, ea, ez, eq, qf, mC, Pm, q, jY, qC, ai, hX, hd, bEk, aE, bpN, Db, fh, ds, oW, aT, cU, cy, bEP, dV, h, K, vO, bEQ, ly, eG, OV, iU, iZ, LC, aW, lW, eH, bEj, eZ, r, aq, xH, S, aFU, t, eF, bz, iK, dr, lx, wA, yd, bw, jV, zr, eM, bE, iT, MB, byG, adR, aFp, dW, aS, m, fl, oX, CI, lC, bt, ei, B, y, BA, L); #c1(GPI) c2(XP_006723211) c3(glucose-6-phosphate isomerase isoform XI) c4( )c5(295042, 295043, 295044, 295045, 295046, 295047) c6( )c7(ID, eW, T, dt, dD, qo, aC, ea, ez, eq, qf, mC, Pm, q, jY, qC, ai, hX, hd, bEk, aE, bpN, Db, fh, ds, oW, aT, cU, cy, bEP, dV, h, K, vO, bEQ, ly, &G, OV, iU, iZ, LC, aW, lW, eH, bEj, eZ, r, aq, xH, S, aFU, t, eF, bz, iK, dr, lx, wA, yd, bw, jV, zr, eM, bE, iT, MB, byG, adR, afp, dW, aS, m, fl, oX, CI, lC, bt, ei, B, y, BA, L); #c1(GPI) c2(XP_011525056) c3(glucose-6-phosphate isomerase isoform X2) c4(319507) c5( )c6( )c7(ID, eW, T, dt, dD, qo, aC, ea, ez, eq, qf, mC, Pm, q, jY, qC, ai, hX, hd, bEk, aE, bpN, Db, fh, ds, oW, aT, cU, cy, bEP, dV, h, K, vO, bEQ, ly, eG, OV, iU, iZ, LC, aW, lW, eH, bEj, eZ, r, aq, xH, S, aFU, t, eF, bz, iK, dr, lx, wA, yd, bw, jV, zr, eM, bE, iT, MB, byG, adR, afp, dW, aS, m, fl, oX, CI, lC, bt, ei, B, y, BA, L); #c1(GPI) c2(XP_011525057) c3(glucose-6-phosphate isomerase isoform X3) c4(319508) c5(319509, 319510, 319511, 319512, 319513, 319514) c6( ) c7(ID, eW, T, dt, dD, qo, aC, ea, ez, eq, qf, mC, Pm, q, jY, qC, ai, hX, hd, bEk, aE, bpN, Db, fh, ds, oW, aT, cU, cy, bEP, dV, h, K, vO, bEQ, ly, eG, OV, iU, iZ, LC, aW, lW, eH, bEj, eZ, r, aq, xH, S, aFU, t, eF, bz, iK, dr, lx, wA, yd, bw, jV, zr, eM, bE, iT, MB, byG, adR, afp, dW, aS, m, fl, oX, CI, IC, bt, ei, B, y, BA, L); #c1(GPIHBP1) c2(NP_835466) c3 (glycosylphosphatidylinositol-anchored high density lipoprotein-binding protein 1 isoform 1 precursor) c4(253532) c5(253533, 253534, 253535, 253536, 253537, 253538) c6( )c7(aW, aT, vG, n, K, ae, Zx, ad); #c1(GPKOW) c2(NP_056513) c3(G patch domain and KOW motifs-containing protein) c4(187872) c5(187873, 187874, 187875, 187876, 187877, 187878, 187879, 187880) c6(187881, 187882) c7(cr); #c1(GPLD1) c2(NP_001494) c3(phosphatidylinositol-glycan-specific phospholipase D precursor) c4(118185, 118186) c5(118187, 118188, 118189, 118190, 118191, 118192) c6( )c7(T, cK, nM, Db, Y, fc, Fx, ei, ee, t); #c1(GPLD1) c2(XP_011512811) c3(phosphatidylinositol-glycan-specific phospholipase D isoform X2) c4( )c5(303874, 303875, 303876, 303877, 303878, 303879) c6( )c7(T, cK, nM, Db, Y, fc, Fx, ei, ee, t); #c1(GPM6A) c2(NP_001248377) c3(neuronal membrane glycoprotein M6-a isoform 4) c4(93898) c5(93899, 93900, 93901, 93902, 93903, 93904) c6( )c7(sv, bS, bK, K, ct, Z, hH, cd, jb); #c1(GPM6A) c2(NP_963885) c3(neuronal membrane glycoprotein M6-a isoform 1) c4(262503, 262504) c5(262505, 262506, 262507, 262508, 262509, 262510, 262511, 262512, 262513, 262514, 262515, 262516, 262517, 262518, 262519, 262520, 262521, 262522, 262523) c6( )c7(sv, bS, bK, K, ct, Z, hH, cd, jb); #c1(GPMGA) c2(NP_963886) c3(neuronal membrane glycoprotein M6-a isoform 3) c4(262524) c5(262525, 262526, 262527, 262528, 262529, 262530) c6( )c7(sv, bS, bK, K, ct, Z, hH, cd, jb); #c1(GPM6B) c2(NP_001001994) c3(neuronal membrane glycoprotein M6-b isoform 4) c4(19290) c5(19291, 19292, 19293, 19294, 19295, 19296, 19297, 19298, 19299, 19300, 19301) c6( )c7(cb, Y, aR, bee, qb); #c1(GPMGB) c2(NP_001001995) c3(neuronal membrane glycoprotein MG-b isoform 1) c4(19302, 19303) c5(19304, 19305, 19306, 19307, 19308, 19309) c6( )c7(cb, Y, aR, bee, qb); #c1(GPM6B) c2(NP_001001996) c3(neuronal membrane glycoprotein M6-b isoform 2) c4(19310) c5(19311, 19312, 19313, 19314, 19315, 19316) c6( ) c7(cb, Y, aR, bee, qb); #c1(GPM6B) c2(NP_005269) c3(neuronal membrane glycoprotein M6-b isoform 3) c4(154614, 154615) c5(154616, 154617, 154618, 154619, 154620, 154621, 154622, 154623, 154624, 154625, 154626, 154627, 154628) c6( ) c7(cb, Y, aR, bee, qb); #c1(GPM6B) c2(XP_005274546) c3(neuronal membrane glycoprotein M6-b isoform X1) c4(285158) c5( ) c6( ) c7(cb, Y, aR, bee, qb); #c1(GPM6B) c2(XP_011543799) c3(neuronal membrane glycoprotein M6-b isoform X3) c4( ) c5(342916, 342917, 342918, 342919, 342920, 342921) c6( ) c7(cb, Y, aR, bee, qb); #c1(GPN1) c2(NP_00138519) c3(GPN-loop GTPase 1 isoform b) c4(56933) c5(56934, 56935, 56936, 56937, 56938, 56939) c6( ) c7(om); #c1(GPN1) c2(NP_001138520) c3(GPN-loop GTPase 1 isoform c) c4(56940) c5(56941, 56942, 56943, 56944, 56945) c6( ) c7(om); #c1(GPN1) c2(NP_001138521) c3(GPN-loop GTPase 1 isoform d) c4(56946) c5(56947, 56948, 56949, 56950, 56951, 56952) c6( ) c7(om); #c1(GPN1) c2(NP_009197) c3(GPN-loop GTPase 1 isoform a) c4(171452) c5(171453, 171454, 171455, 171456, 171457, 171458, 171459) c6( ) c7(om); #c1(GPNMB) c2(NP_001005340) c3(transmembrane glycoprotein NMB isoform a precursor) c4(21417, 21418, 21419, 21420) c5(21421, 21422, 21423, 21424, 21425, 21426, 21427, 21428, 21429, 21430, 21431, 21432, 21433, 21434, 21435, 21436, 21437, 21438, 21439, 21440, 21441, 21442, 21443, 21444, 21445) c6( ) c7(oO, T, yS, al, n, v, yO, ci, qN, q, ad, eA, bER, aE, aT, h, ey, fb, ae, cp, afu, hM, yo, aW, Y, r, aq, Tp, g, t, f, ck, es, gk, Dy, iA, dB, avZ, E, m, pJ, B); #c1(GPNMB) c2(NP_002501) c3(transmembrane glycoprotein NMB isoform b precursor) c4(129356, 129357) c5(129358, 129359, 129360, 129361, 129362, 129363, 129364, 129365, 129366, 129367, 129368, 129369) c6( ) c7(oO, T, yS, al, n, v, yO, ci, qN, q, ad, eA, bER, aE, aT, h, ey, fb, ae, cp, afu, hM, yo, aW, Y, r, aq, Tp, g, t, f, ck, es, gk, Dy, iA, dB, avZ, E, m, pJ, B); #c1(GPNMB) c2(XP_005249635) c3(transmembrane glycoprotein NMB isoform X1) c4(269764) c5(269765, 269766, 269767, 269768, 269769, 269770) c6( ) c7(oO, T, yS, al, n, v, yO, ci, qN, q, ad, eA, bER, aE, aT, h, ey, fb, ae, cp, afu, hM, yo, aW, Y, r, aq, Tp, g, t, f, ck, es, gk, Dy, iA, dB, avZ, E, m, pJ, B); #c1(GPR1) c2(NP_001091669) c3(G-protein coupled receptor 1) c4(38555, 38556, 38557, 38558, 38559, 38560, 38561) c5(38562, 38563, 38564, 38565, 38566, 38567, 38568, 38569, 38570, 38571, 38572, 38573, 38574, 38575, 38576, 38577, 38578, 38579, 38580, 38581, 38582, 38583, 38584, 38585, 38586, 38587, 38588, 38589, 38590, 38591, 38592, 38593, 38594, 38595, 38596, 38597, 38598, 38599, 38600, 38601) c6( ) c7(h, dy); #c1(GPR1D1) c2(NP_473362) c3(probable G-protein coupled receptor 101) c4(230389) c5(230390, 230391, 230392, 230393, 230394, 230395) c6( ) c7(bES, OB, oe); #c1(GPR119) c2(NP_848566) c3(glucose-dependent insulinotropic receptor) c4(253871) c5(253872, 253873, 253874, 253875, 253876, 253877) c6( ) c7(bL, dD, ea, dq, aj, v, eq, J); #c1(GPR12) c2(NP_005279) c3(G-protein coupled receptor 12) c4(154656) c5(154657, 154658, 154659, 154660, 154661, 154662, 154663, 154664, 154665, 154666) c6( ) c7(bM, v, cd, iV); #c1(GPR132) c2(NP_001265623) c3(probable G-protein coupled receptor 132 isoform 1) c4(99035) c5(99036, 99037, 99038, 99039, 99040, 99041, 99042, 99043, 99044, 99045, 99046, 99047, 99048, 99049) c6( ) c7(dD, eq, eZ, en, ff); #c1(GPR132) c2(NP_001265624) c3(probable G-protein coupled receptor 132 isoform 2) c4(99050) c5(99051, 99052, 99053, 99054, 99055, 99056) c6( ) c7(dD, eq, eZ, en, ff); #c1(GPR135) c2(NP_072093) c3(probable G-protein coupled receptor 135) c4(212677, 212678) c5(212679, 212680, 212681, 212682, 212683, 212684, 212685, 212686, 212687, 212688, 212689, 212690, 212691, 212692, 212693, 212694) c6( ) c7(z); #c1(GPR137C) c2(NP_001093122) c3(integral membrane protein GPR137C) c4(39794) c5(39795, 39796, 39797, 39798, 39799, 39800) c6( ) c7(cd); #c1(GPR139) c2(NP_001002911) c3(probable G-protein coupled receptor 139) c4(19741) c5(19742, 19743, 19744, 19745, 19746, 19747, 19748, 19749, 19750, 19751) c6( ) c7(fq); #c1(GPR143) c2(NP_000264) c3(G-protein coupled receptor 143) c4(5923, 5924, 5925, 5926) c5(5927, 5928, 5929, 5930, 5931, 5932, 5933, 5934, 5935, 5936, 5937, 5938) c6( ) c7(KE, aAd, Y, NG, oQ, bET); #c1(GPR143) c2(XP_005274598) c3(G-protein coupled receptor 143 isoform X1) c4( ) c5(285174, 285175, 285176, 285177, 285178, 285179, 285180, 285181, 285182, 285183, 285184, 285185) c6( ) c7(KE, aAd, Y, NG, oQ, bET); #c1(GPR148) c2(NP_997247) c3(probable G-protein coupled receptor 148) c4(265311) c5(265312, 265313, 265314, 265315, 265316, 265317) c6( ) c7(K); #c1(GPR149) c2(NP_001033794) c3(probable G-protein coupled receptor 149) c4(31567) c5(31568, 31569, 31570, 31571, 31572, 31573) c6( ) c7(fq); #c1(GPR15) c2(NP_005281) c3(G-protein coupled receptor 15) c4(154667) c5(154668, 154669, 154670, 154671, 154672, 154673) c6( ) c7(h, y, bX); #c1(GPR150) c2(NP_954713) c3(probable G-protein coupled receptor 150) c4(261354) c5(261355, 261356, 261357, 261358, 261359, 261360) c6( ) c7(t); #c1(GPR151) c2(NP_919227) c3(probable G-protein coupled receptor 151) c4(258268) c5(258269, 258270, 258271, 258272, 258273, 258274) c6( ) c7(bL, bM, T, d, aE, Y, pX, ff, h, Ja, v, dX, en, he, Fg, cE); #c1(GPR152) c2(NP_996880) c3(probable G-protein coupled receptor 152) c4(264789) c5(264790, 264791, 264792, 264793, 264794, 264795, 264796) c6( ) c7(W); #c1(GPR153) c2(XP_011539736) c3(probable G-protein coupled receptor 153 isoform X1) c4(337956) c5(337957, 337958, 337959, 337960, 337961, 337962, 337963) c6( ) c7(g, hS); #c1(GPR155) c2(NP_001253980) c3(integral membrane protein GPR155 isoform 2) c4( ) c5(94348, 94349, 94350, 94351, 94352, 94353) c6( ) c7(bN); #c1(GPR155) c2(NP_689742) c3(integral membrane protein GPR155 isoform 1) c4(243816, 243817) c5(243818, 243819, 243820, 243821, 243822, 243823, 243824, 243825, 243826, 243827, 243828, 243829, 243830, 243831) c6( ) c7(bN); #c1(GPR156) c2(NP_001161743) c3(probable G-protein coupled receptor 156 isoform 2) c4(65895) c5(65896, 65897, 65898, 65899, 65900, 65901) c6( ) c7(cF, ac, an); #c1(GPR156) c2(NP_694547) c3(probable G-protein coupled receptor 156 isoform 1) c4(245118) c5(245119, 245120, 245121, 245122, 245123, 245124, 245125, 245126, 245127, 245128) c6( ) c7(cF, ac, an); #c1(GPR158) c2(NP_065803) c3(probable G-protein coupled receptor 158 precursor) c4(205610) c5(205611, 205612, 205613, 205614, 205615, 205616) c6( ) c7(ht, ff, j, en); #c1(GPR160) c2(NP_055188) c3(probable G-protein coupled receptor 160) c4(179653, 179654, 179655, 179656) c5(179657, 179658, 179659, 179660, 179661, 179662, 179663, 179664, 179665, 179666, 179667, 179668, 179669, 179670, 179671, 179672, 179673, 179674, 179675, 179676, 179677, 179678, 179679) c6( ) c7(T, ac); #c1(GPR160) c2(XP_011510981) c3(probable G-protein coupled receptor 160 isoform X2) c4( ) c5(301429) c6( ) c7(T, ac); #c1(GPR160) c2(XP_011510982) c3(probable G-protein coupled receptor 160 isoform X3) c4( ) c5(301430, 301431) c6( ) c7(T, ac); #c1(GPR161) c2(NP_001254540) c3(G-protein coupled receptor 161 isoform 3) c4(94621) c5(94622) c6( ) c7(bA, cz); #c1(GPR161) c2(NP_001254542) c3(G-protein coupled receptor 161 isoform 5) c4(94623) c5(94624, 94625, 94626, 94627, 94628, 94629) c6( ) c7(bA, cz); #c1(GPR161) c2(NP_001254543) c3(G-protein coupled receptor 161 isoform 6) c4(94630) c5(94631, 94632, 94633, 94634, 94635, 94636) c6( ) c7(bA, cz); #c1(GPR161) c2(XP_005245112) c3(G-protein coupled receptor 161 isoform X1) c4(266301) c5(266302, 266303, 266304, 266305, 266306, 266307) c6( ) c7(bA, cz); #c1(GPR161) c2(XP_011507676) c3(G-protein coupled receptor 161 isoform X3) c4( ) c5(297681, 297682) c6( ) c7(bA, cz); #c1(GPR161) c2(XP_011507680) c3(G-protein coupled receptor 161 isoform X2) c4( ) c5(297683, 297684, 297685, 297686, 297687, 297688, 297689, 297690, 297691, 297692, 297693, 297694, 297695, 297696) c6( ) c7(bA, cz); #c1(GPR162) c2(NP_055264) c3(probable G-protein coupled receptor 162 isoform 1) c4( ) c5(180141, 180142, 180143, 180144, 180145, 180146) c6( ) c7(dD, ef, ea, aw, aj, j, eq, ee, q, J, ad, GK, Zu, dQ, ae, uQ, Zt, r, aq, iO, Xa, WX, qR, B); #c1(GPR162) c2(NP_062832) c3(probable G-protein coupled receptor 162 isoform 2) c4(202186, 202187) c5(202188, 202189, 202190, 202191, 202192, 202193) c6( ) c7(dD, ef, ea, aw, aj, j, eq, ee, q, J, ad, GK, Zu, dQ, ae, uQ, Zt, r, aq, iO, Xa, WX, qR, B); #c1(GPR17) c2(NP_001154889) c3(uracil nucleotide/cysteinyl leukotriene receptor isoform b) c4(61207) c5(61208, 61209, 61210, 61211, 61212) c6( ) c7(wO, wg, cy); #c1(GPR17) c2(NP_005282) c3(uracil nucleotide/cysteinyl leukotriene receptor isoform a) c4(154674, 154675) c5(154676, 154677, 154678, 154679, 154680, 154681, 154682, 154683, 154684, 154685, 154686, 154687, 154688, 154689, 154690, 154691, 154692, 154693, 154694, 154695, 154696) c6( ) c7(wO, wg, cy); #c1(GPR171) c2(NP_037440) c3(probable G-protein coupled receptor 171) c4(175530, 175531) c5(175532, 175533, 175534, 175535, 175536, 175537, 175538, 175539, 175540, 175541, 175542, 175543) c6( ) c7(oc); #c1(GPR174) c2(NP_115942) c3(probable G-protein coupled receptor 174) c4(224829) c5(224830, 224831, 224832, 224833, 224834, 224835) c6( ) c7(hD); #c1(GPR176) c2(NP_001258784) c3(probable G-protein coupled receptor 176 isoform 3) c4(96528) c5(96529, 96530, 96531, 96532, 96533, 96534) c6( ) c7(cF, oe, Hr, fq); #c1(GPR176) c2(NP_009154) c3(probable G-protein coupled receptor 176 isoform 1) c4(171178) c5(171179, 171180, 171181, 171182, 171183, 171184, 171185, 171186, 171187) c6( ) c7(cF, oe, Hr, fq); #c1(GPR179) c2(NP_001004334) c3(probable G-protein coupled receptor 179 precursor) c4(20605) c5(20606, 20607, 20608, 20609, 20610, 20611) c6( ) c7(bEU); #c1(GPR180) c2(NP_851320) c3(integral membrane protein GPR180 precursor) c4(254541) c5(254542, 254543, 254544, 254545, 254546, 254547) c6( ) c7(Ae, kT); #c1(GPR182) c2(XP_011536129) c3(G-protein coupled receptor 182 isoform X1) c4(333434) c5(333435, 333436, 333437, 333438, 333439, 333440) c6( ) c7(aE, aW, ea, cz, aT, at, azl, lC); #c1(GPR183) c2(NP_004942) c3(G-protein coupled receptor 183) c4(151332) c5(151333, 151334, 151335, 151336, 151337, 151338) c6( ) c7(A, ey); #c1(GPR19) c2(XP_011518926) c3(probable G-protein coupled receptor 19 isoform X2) c4(313366, 313367) c5(313368, 313369, 313370) c6( ) c7(bz); #c1(GPR19) c2(XP_011518927) c3(probable G-protein coupled receptor 19 isoform X1) c4( ) c5(313371, 313372, 313373, 313374, 313375, 313376, 313377, 313378, 313379, 313380, 313381, 313382, 313383) c6( ) c7(bz); #c1(GPR20) c2(NP_005284) c3(G-protein coupled receptor 20) c4(154697) c5(154698, 154699, 154700, 154701, 154702, 154703) c6( ) c7(bL); #c1(GPR22) c2(XP_011514358) c3(probable G-protein coupled receptor 22 isoform X1) c4(307057) c5(307058, 307059, 307060, 307061, 307062, 307063) c6( ) c7(Dg, Dj, K); #c1(GPR26) c2(NP_703143) c3(G-protein coupled receptor 26) c4(245969) c5(245970, 245971, 245972, 245973, 245974, 245975) c6( ) c7(d, bS, ct, Z, eA, hq); #c1(GPR34) c2(NP_001091048) c3(probable G-protein coupled receptor 34) c4(38370, 38371) c5(38372, 38373, 38374, 38375, 38376, 38377, 38378, 38379, 38380, 38381, 38382, 38383, 38384, 38385, 38386, 38387) c6( ) c7(dB, iW, jh); #c1(GPR35) c2(NP_001182311) c3(G-protein coupled receptor 35 isoform b) c4(74763, 74764) c5(74765, 74766, 74767, 74768, 74769, 74770, 74771, 74772, 74773) c6( ) c7(bL, bri, hN, lS, iV, EJ); #c1(GPR35) c2(NP_005292) c3(G-protein coupled receptor 35 isoform a) c4( ) c5(154719, 154720, 154721, 154722, 154723, 154724, 154725, 154726, 154727, 154728, 154729, 154730, 154731, 154732) c6( ) c7(bL, bri, hN, lS, iV, EJ); #c1(GPR37) c2(NP_005293) c3(prosaposin receptor GPR37 precursor) c4(154733) c5(154734, 154735, 154736, 154737, 154738, 154739) c6( ) c7(bgk, vj, K, an, rz, bt); #c1(GPR37L1) c2(NP_004758) c3(prosaposin receptor GPR37L1 precursor) c4(149365, 149366) c5(149367, 149368, 149369, 149370, 149371, 149372, 149373, 149374, 149375, 149376, 149377, 149378) c6( ) c7(gZ); #c1(GPR39) c2(NP_001499) c3(G-protein coupled receptor 39) c4(118214) c5(118215, 118216, 118217, 118218, 118219, 118220, 118221) c6( ) c7(bL, vj, ht, it, v, Ea, hu, ae, cd, ad); #c1(GPR4) c2(NP_005273) c3(G-protein coupled receptor 4) c4(154629) c5(154630, 154631, 154632, 154633, 154634, 154635) c6( ) c7(T, aPm, K, m, t, bz); #c1(GPR50) c2(NP_004215) c3(melatonin-related receptor) c4(144346, 144347) c5(144348, 144349, 144350, 144351, 144352, 144353, 144354, 144355, 144356, 144357, 144358, 144359) c6( ) c7(VX, cA, vj, v, bK, bQ, rz); #c1(GPR52) c2(NP_005675) c3(probable G-protein coupled receptor 52) c4(158429) c5(158430, 158431, 158432, 158433, 158434, 158435) c6( ) c7(gl, ayR); #c1(GPR55) c2(XP_011510479) c3(G-protein coupled receptor 55 isoform X1) c4(300984, 300985) c5(300986, 300987, 300988, 300989, 300990, 300991, 300992, 300993, 300994, 300995, 300996, 300997, 300998, 300999, 301000, 301001, 301002, 301003, 301004, 301005) c6( ) c7(bL, T, hk, yk, ff, v, ci, m, en, t, jb); #c1(GPR6) c2(NP_001273028) c3(G-protein coupled receptor 6 isoform a) c4(104185, 104186) c5(104187, 104188, 104189, 104190, 104191, 104192) c6( ) c7(cF); #c1(GPR6) c2(NP_005275) c3(G-protein coupled receptor 6 isoform b) c4(154636) c5(154637, 154638, 154639, 154640, 154641, 154642, 154643, 154644, 154645, 154646, 154647, 154648) c6( ) c7(cF); #c1(GPR65) c2(NP_003599) c3(psychosine receptor) c4(139481) c5(139482, 139483, 139484, 139485, 139486, 139487) c6( ) c7(T, aw, ey, bK, V, ad); #c1(GPR68) c2(NP_001171147) c3(ovarian cancer G-protein coupled receptor 1) c4( ) c5(69949, 69950, 69951, 69952, 69953, 69954, 69955, 69956, 69957, 69958, 69959, 69960, 69961) c6( ) c7(t, ae, m, ad); #c1(GPR68) c2(XP_011535502) c3(ovarian cancer G-protein coupled receptor 1 isoform X1) c4(332548, 332549, 332550) c5(332551, 332552, 332553, 332554, 332555, 332556, 332557, 332558, 332559) c6( ) c7(t, ae, m, ad); #c1(GPR75) c2(NP_006785) c3(probable G-protein coupled receptor 75) c4(168052) c5(168053, 168054, 168055, 168056, 168057, 168058) c6( ) c7(W); #c1(GPR78) c2(NP_543009) c3(G-protein coupled receptor 78) c4(233222) c5(233223, 233224, 233225, 233226, 233227, 233228) c6( ) c7(bK, vi); #c1(GPR83) c2(NP_057624) c3(probable G-protein coupled receptor 83 precursor) c4(191926) c5(191927, 191928, 191929, 191930, 191931, 191932) c6( ) c7(v, aZ, mf); #c1(GPR87) c2(NP_076404) c3(G-protein coupled receptor 87) c4(214055) c5(214056, 214057, 214058, 214059, 214060, 214061) c6( ) c7(vj, ff, en); #c1(GPRASP1) c2(NP_001092881) c3(G-protein coupled receptor-associated sorting protein 1) c4(39705) c5(39706, 39707, 39708, 39709, 39710, 39711, 39712, 39713, 39714, 39715, 39716, 39717, 39718, 39719, 39720, 39721, 39722, 39723) c6( ) c7(bM); #c1(GPRC5A) c2(NP_003970) c3(retinoic acid-induced protein 3) c4(142530, 142531) c5(142532, 142533, 142534, 142535, 142536, 142537, 142538, 142539, 142540, 142541, 142542, 142543, 142544, 142545) c6(142546, 142547) c7(T, aA, bB, ci, ad, av, iu, ft, jz, aT, jj, ae, Yi, aa, lU, iU, MD, pr, aW, ff, cr, gE, aF, en, cl, g, cm, bz, ck, bF, eM, bE, cj, at, fD, it, B); #c1(GPRC5B) c2(NP_001291700) c3(G-protein coupled receptor family C group 5 member B isoform 2) c4(116362) c5(116363, 116364, 116365, 116366, 116367, 116368) c6( ) c7(Jd, bL, fq, v, FE); #c1(GPRC5B) c2(XP_006721115) c3(G-protein coupled receptor family C group 5 member B isoform X1) c4(293840, 293841, 293842) c5(293843, 293844, 293845, 293846, 293847, 293848, 293849, 293850, 293851, 293852, 293853, 293854, 293855, 293856, 293857, 293858, 293859, 293860) c6( ) c7(Jd, bL, fq, v, FE); #c1(GPRC5C) c2(NP_061123) c3(G-protein coupled receptor family C group 5 member C isoform b precursor) c4(199245) c5(199246, 199247, 199248, 199249, 199250, 199251, 199252, 199253, 199254, 199255, 199256) c6( ) c7(bEV, bM, ft, alu, cA, aW, aT, it, fd, bz, B); #c1(GPRC5C) c2(NP_071319) c3(G-protein coupled receptor family C group 5 member C isoform a) c4(210933, 210934) c5(210935, 210936, 210937, 210938, 210939, 210940, 210941) c6( ) c7(bEV, bM, ft, alu, cA, aW, aT, it, fd, bz, B); #c1(GPRC5C) c2(XP_005257571) c3(G-protein coupled receptor family C group 5 member C isoform X4) c4( ) c5(275268, 275269, 275270, 275271, 275272, 275273) c6( ) c7(bEV, bM, ft, alu, cA, aW, aT, it, fd, bz, B); #c1(GPRC5C) c2(XP_011523324) c3(G-protein coupled receptor family C group 5 member C isoform X1) c4(317864) c5( ) c6( ) c7(bEV, bM, ft, alu, cA, aW, aT, it, fd, bz, B); #c1(GPRC5C) c2(XP_011523325) c3(G-protein coupled receptor family C group 5 member C isoform X2) c4(317865) c5(317866, 317867, 317868, 317869, 317870, 317871) c6( ) c7(bEV, bM, ft, alu, cA, aW, aT, it, fd, bz, B); #c1(GPRC5D) c2(NP_061124) c3(G-protein coupled receptor family C group 5 member D) c4(199257, 199258) c5(199259, 199260, 199261, 199262, 199263, 199264) c6( ) c7(bEV, bM, T, ft, alu, cA, aW, vj, aT, it, fd, bz, B); #c1(GPRC5D) c2(XP_011519048) c3(G-protein coupled receptor family C group 5 member D isoform X2) c4(313543) c5(313544, 313545, 313546, 313547, 313548, 313549, 313550, 313551, 313552, 313553, 313554, 313555) c6( ) c7(bEV, bM, T, ft, alu, cA, aW, vj, aT, it, fd, bz, B); #c1(GPRC6A) c2(NP_001273283) c3(G-protein coupled receptor family C group 6 member A isoform 2 precursor) c4(104607) c5(104608, 104609, 104610, 104611, 104612, 104613) c6( ) c7(bL, bM, d, aE, Y, pX, cE, ff, h, Ja, v, dX, en, he, Fg, ae, ad); #c1(GPRC6A) c2(NP_001273284) c3(G-protein coupled receptor family C group 6 member A isoform 3 precursor) c4(104614, 104615, 104616, 104617) c5(104618, 104619, 104620, 104621, 104622, 104623) c6( ) c7(bL, bM, d, aE, Y, pX, cE, ff, h, Ja, v, dX, en, he, Fg, ae, ad); #c1(GPRC6A) c2(NP_683766) c3(G-protein coupled receptor family C group 6 member A isoform 1 precursor) c4(242579, 242580, 242581, 242582) c5(242583, 242584, 242585, 242586, 242587, 242588, 242589) c6( ) c7(bL, bM, d, aE, Y, pX, cE, ff, h, Ja, v, dX, en, he, Fg, ae, ad); #c1(GPRIN1) c2(XP_005265863) c3(G protein-regulated inducer of neurite outgrowth 1 isoform X1) c4(279804, 279805) c5(279806, 279807, 279808, 279809, 279810, 279811, 279812, 279813, 279814, 279815, 279816, 279817) c6( ) c7(FX, v, bK, cC); #c1(GPRIN2) c2(XP_011538700) c3(G protein-regulated inducer of neurite outgrowth 2 isoform X2) c4(336456) c5(336457, 336458) c6( ) c7(bk, bO); #c1(GPRIN2) c2(XP_011538702) c3(G protein-regulated inducer of neurite outgrowth 2 isoform X1) c4( ) c5(336459, 336460, 336461, 336462, 336463, 336464, 336465, 336466, 336467, 336468, 336469) c6( ) c7(bk, bO); #c1(GPRIN3) c2(XP_005262993) c3(G protein-regulated inducer of neurite outgrowth 3 isoform X1) c4(278191) c5(278192, 278193, 278194, 278195, 278196, 278197) c6( ) c7(aw, an); #c1(GPS2) c2(NP_004480) c3(G protein pathway suppressor 2) c4(146750, 146751) c5(146752, 146753, 146754, 146755, 146756, 146757, 146758, 146759, 146760, 146761, 146762, 146763, 146764, 146765, 146766, 146767, 146768, 146769, 146770) c6( ) c7(h, Y, v, qM); #c1(GPSM1) c2(NP_001139110) c3(G-protein-signaling modulator 1 isoform a) c4(58681, 58682) c5(58683, 58684, 58685, 58686, 58687, 58688, 58689, 58690, 58691) c6( ) c7(cu, aBZ, Nj, wR, wB); #c1(GPSM1) c2(NP_001186932) c3(G-protein-signaling modulator 1 isoform c) c4(78824) c5(78825, 78826, 78827, 78828, 78829, 78830, 78831, 78832, 78833, 78834, 78835) c6( ) c7(cu, aBZ, Nj, wR, wB); #c1(GPSM1) c2(NP_056412) c3(G-protein-signaling modulator 1 isoform b) c4(187491) c5(187492, 187493, 187494, 187495, 187496, 187497) c6( ) c7(cu, aBZ, Nj, wR, wB); #c1(GPSM1) c2(XP_011516800) c3(G-protein-signaling modulator 1 isoform X1) c4( ) c5(310254, 310255, 310256, 310257, 310258, 310259) c6( ) c7(cu, aBZ, Nj, wR, wB); #c1(GPSM2) c2(XP_011539605) c3(G-protein-signaling modulator 2 isoform X3) c4( ) c5(337825, 337826, 337827, 337828, 337829, 337830, 337831, 337832, 337833, 337834, 337835, 337836, 337837, 337838, 337839, 337840, 337841, 337842) c6( ) c7(bEW, bF, T, aW, FB, mG, aT, Bh); #c1(GPSM3) c2(NP_071390) c3(G-protein-signaling modulator 3) c4(211359) c5(211360, 211361, 211362, 211363, 211364, 211365, 211366, 211367, 211368, 211369, 211370, 211371, 211372, 211373, 211374, 211375, 211376, 211377, 211378, 211379, 211380, 211381, 211382, 211383, 211384, 211385, 211386, 211387, 211388, 211389, 211390, 211391, 211392, 211393, 211394, 211395, 211396, 211397, 211398, 211399, 211400, 211401, 211402, 211403, 211404, 211405, 211406, 211407) c6( ) c7(eZ, cr, iV); #c1(GPT) c2(XP_011515295) c3(alanine aminotransferase 1 isoform X1) c4(308164) c5(308165, 308166, 308167, 308168, 308169, 308170, 308171, 308172, 308173, 308174) c6( ) c7(ck, bv, D, jx, hl, bu, wc, fQ, cy, aq, mf, aNp, K, fc, avG, B); #c1(GPT2) c2(NP_001135938) c3(alanine aminotransferase 2 isoform 2) c4( ) c5(53626, 53627, 53628, 53629, 53630) c6( ) c7(aNp, hl, K, fc); #c1(GPT2) c2(NP_597700) c3(alanine aminotransferase 2 isoform 1) c4(235042, 235043) c5(235044, 235045, 235046, 235047, 235048, 235049, 235050) c6( ) c7(aNp, hl, K, fc); #c1(GPX1) c2(NP_000572) c3(glutathione peroxidase 1 isoform 1) c4(12817) c5(12818, 12819, 12820, 12821, 12822, 12823, 12824) c6( ) c7(bM, T, aC, ea, e, al, bB, fo, ip, K, qM, eL, xa, aW, fc, hl, bEX, g, cm, wT, mx, mk, lS, dD, gU, zt, aw, n, aj, PJ, TB, axc, h, lG, jy, dL, KV, cM, kT, W, dB, hk, at, IB, B, bL, eR, Dh, cz, hy, v, j, bX, q, J, eA, uT, afQ, aT, cy, bq, an, ae, af, eu, bA, s, Y, r, HL, en, bN, ac, hr, im, oq, id, iW, ha, jh, dG, Gn, eq, ad, kL, av, HF, ji, uX, ff, bz, qa, D, gk, pu, bE, aS, be, ei, rk); #c1(GPX1) c2(NP_958799) c3(glutathione peroxidase 1 isoform 2) c4(262199) c5(262200, 262201, 262202, 262203, 262204) c6( ) c7(bM, T, aC, ea, e, al, bB, fo, ip, K, qM, eL, xa, aW, fc, hl, bEX, g, cm, wT, mx, mk, lS, dD, gU, zt, aw, n, aj, PJ, TB, axc, h, lG, jy, dL, KV, cM, kT, W, dB, hk, at, lB, B, bL, eR, Dh, cz, hy, v, j, bX, q, J, eA, uT, afQ, aT, cy, bq, an, ae, af, eu, bA, s, Y, r, HL, en, bN, ac, hr, im, oq, id, iW, ha, jh, dG, Gn, eq, ad, kL, av, HF, ji, uX, ff, bz, qa, D, gk, pu, bE, aS, be, ei, rk); #c1(GPX2) c2(NP_002074) c3(glutathione peroxidase 2) c4(124272) c5(124273, 124274, 124275, 124276, 124277, 124278) c6( ) c7(T, pf, Dh, iW, NT, bB, n, ci, q, ad, azn, aT, dL, ji, KV, ae, s, aW, LZ, r, g, bz, bE, at, B); #c1(GPX3) c2(NP_002075) c3(glutathione peroxidase 3 precursor) c4(124279, 124280) c5(124281, 124282, 124283, 124284, 124285, 124286, 124287, 124288, 124289, 124290, 124291, 124292) c6( ) c7(bL, bM, T, dD, dA, iW, e, NT, aA, bB, aj, v, eq, ci, fr, lu, ad, dP, av, b, awd, M, aT, h, dL, bK, K, bq, nJ, ae, aa, xQ, d, s, MD, qt, aW, Y, az, hl, LZ, uY, aq, vs, aF, g, mo, f, bw, qa, ck, ac, c, nB, bE, iA, U, hL, at, m, i, it, B); #c1(GPX4) c2(NP_002076) c3(phospholipid hydroperoxide glutathione peroxidase, mitochondrial isoform A precursor) c4(124293) c5(124294, 124295, 124296, 124297, 124298, 124299) c6( ) c7(bL, bM, oq, T, gU, fM, dD, ea, iW, bmk, oR, arw, eq, fr, AO, cd, q, ad, av, JT, aT, dL, ae, A, s, aW, Y, ff, hl, r, en, t, bz, qa, ac, at, dy, im, iu); #c1(GPX5) c2(NP_001500) c3(epididymal secretory glutathione peroxidase isoform 1 precursor) c4(118222) c5(118223, 118224, 118225, 118226, 118227, 118228, 118229, 118230, 118231, 118232, 118233, 118234, 118235, 118236) c6(118237, 118238) c7(fq); #c1(GPX5) c2(NP_003987) c3(epididymal secretory glutathione peroxidase isoform 2 precursor) c4(142662) c5(142663, 142664, 142665, 142666, 142667, 142668, 142669, 142670, 142671) c6(142672) c7(fq); #c1(GRAMD1B) c2(NP_001273492) c3(GRAM domain-containing protein (B isoform 1) c4(105052) c5(105053, 105054, 105055, 105056, 105057, 105058) c6( ) c7(cj); #c1(GRAMD1B) c2(NP_065767) c3(GRAM domain-containing protein 1B isoform 2) c4(205275, 205276) c5(205277, 205278, 205279, 205280, 205281, 205282, 205283, 205284, 205285, 205286, 205287, 205288) c6( ) c7(cj); #c1(GRAMD3) c2(NP_001139791) c3(GRAM domain-containing protein 3 isoform 1) c4(59923) c5(59924, 59925, 59926, 59927, 59928, 59929, 59930) c6( ) c7(fK); #c1(GRAMD3) c2(NP_001139792) c3(GRAM domain-containing protein 3 isoform 3) c4( ) c5(59931, 59932, 59933, 59934, 59935, 59936) c6( ) c7(fK); #c1(GRAMD3) c2(NP_001139793) c3(GRAM domain-containing protein 3 isoform 4) c4(59937) c5(59938, 59939, 59940, 59941, 59942, 59943) c6( ) c7(fK); #c1(GRAMD3) c2(NP_001139794) c3(GRAM domain-containing protein 3 isoform 5) c4(59944, 59945) c5(59946, 59947, 59948, 59949, 59950, 59951, 59952, 59953, 59954, 59955, 59956, 59957) c6( ) c7(fK); #c1(GRAMD3) c2(NP_076416) c3(GRAM domain-containing protein 3 isoform 2) c4(214098) c5(214099, 214100, 214101, 214102, 214103, 214104) c6( ) c7(fK); #c1(GRAMD3) c2(XP_005272115) c3(GRAM domain-containing protein 3 isoform X6) c4(283707) c5( ) c6( ) c7(fK); #c1(GRAMD3) c2(XP_005272117) c3(GRAM domain-containing protein 3 isoform X8) c4(283708) c5(283709, 283710, 283711, 283712, 283713, 283714) c6( ) c7(fK); #c1(GRAMD4) c2(NP_055939) c3(GRAM domain-containing protein 4) c4( ) c5(184571, 184572, 184573, 184574, 184575, 184576, 184577, 184578) c6( ) c7(byn, Y, rs, rq); #c1(GRAMD4) c2(XP_005261453) c3(GRAM domain-containing protein 4 isoform X3) c4( ) c5(277360) c6( ) c7(byn, Y, rs, rq); #c1(GRAMD4) c2(XP_006724232) c3(GRAM domain-containing protein 4 isoform X5) c4(295871) c5(295872, 295873) c6( ) c7(byn, Y, rs, rq); #c1(GRAMD4) c2(XP_011528314) c3(GRAM domain-containing protein 4 isoform X4) c4(323375) c5( ) c6( ) c7(byn, Y, rs, rq); #c1(GRAMD4) c2(XP_011528316) c3(GRAM domain-containing protein 4 isoform X7) c4( ) c5(323376, 323377, 323378, 323379, 323380) c6( ) c7(byn, Y, rs, rq); #c1(GRAP) c2(NP_006604) c3(GRB2-related adapter protein) c4(166567, 166568) c5(166569, 166570, 166571, 166572, 166573, 166574, 166575, 166576, 166577, 166578, 166579, 166580) c6( ) c7(gU, fN); #c1(GRAP) c2(XP_005256482) c3(GRB2-related adapter protein isoform X1) c4( ) c5(274187, 274188) c6( ) c7(gU, fN); #c1(GRAP2) c2(NP_001278754) c3(GRB2-related adapter protein 2 isoform 1) c4(110012, 110013, 110014) c5(110015, 110016, 110017, 110018, 110019, 110020, 110021, 110022, 110023, 110024, 110025, 110026, 110027, 110028, 110029, 110030, 110031, 110032, 110033, 110034, 110035, 110036, 110037, 110038, 110039, 110040, 110041, 110042, 110043) c6( ) c7(T, aC, ea, if, fd, bB, gf, lR, Lw, bv, xE, aE, rw, dV, K, aW, zC, g, as, eF, cm, bw, eM, nB, cj, NO, Hb, m, NN, NK, dD, NJ, aj, BF, ct, qN, fr, cd, ai, Hx, jz, cU, h, fe, aq, NL, w, hR, B, bL, Dw, NQ, fM, yk, lH, jx, aA, cR, cz, v, C, V, q, eA, eJ, zn, yw, nv, aT, kf, cy, ae, af, hM, wr, iU, Y, r, lo, en, t, iK, pb, ck, im, bt, ao, kh, jh, dG, eq, iJ, ad, dP, av, CJ, F, ds, ji, lQ, NM, NP, d, ff, eH, cr, aF, bz, bE, y, iu); #c1(GRAP2) c2(NP_001278757) c3(GRB2-related adapter protein 2 isoform 3) c4(110044) c5(110045, 110046, 110047, 110048, 110049, 110050) c6( ) c7(T, aC, ea, if, fd, bB, gf, lR, Lw, bv, xE, aE, rw, dV, K, aW, zC, g, as, eF, cm, bw, eM, nB, cj, NO, Hb, m, NN, NK, dD, NJ, aj, BF, ct, qN, fr, cd, ai, Hx, jz, cU, h, fe, aq, NL, w, hR, B, bL, Dw, NQ, fM, yk, lH, jx, aA, cR, cz, v, C, V, q, eA, eJ, zn, yw, nv, aT, kf, cy, ae, af, hM, wr, iU, Y, r, lo, en, t, iK, pb, ck, im, bt, ao, kh, jh, dG, eq, iJ, ad, dP, av, CJ, F, ds, ji, lQ, NM, NP, d, ff, eH, cr, aF, bz, bE, y, iu); #c1(GRAP2) c2(XP_006724439) c3(GRB2-related adapter protein 2 isoform X2) c4(295968, 295969, 295970) c5(295971, 295972, 295973, 295974, 295975, 295976, 295977, 295978, 295979, 295980, 295981) c6( ) c7(T, aC, ea, if, fd, bB, gf, lR, Lw, bv, xE, aE, rw, dV, K, aW, zC, g, as, eF, cm, bw, eM, nB, cj, NO, Hb, m, NN, NK, dD, NJ, aj, BF, ct, qN, fr, cd, ai, Hx, jz, cU, h, fe, aq, NL, w, hR, B, bL, Dw, NQ, fM, yk, lH, jx, aA, cR, cz, v, C, V, q, eA, eJ, zn, yw, nv, aT, kf, cy, ae, af, hM, wr, iU, Y, r, lo, en, t, iK, pb, ck, im, bt, ao, kh, jh, dG, eq, iJ, ad, dP, av, CJ, F, ds, ji, lQ, NM, NP, d, ff, eH, cr, aF, bz, bE, y, iu); #c1(GRASP) c2(NP_001258785) c3(general receptor for phosphoinositides 1-associated scaffold protein isoform 2) c4(96535) c5(96536, 96537, 96538, 96539, 96540, 96541, 96542, 96543, 96544) c6( ) c7(q, ci, s, r); #c1(GRASP) c2(NP_859062) c3(general receptor for phosphoinositides 1-associated scaffold protein isoform 1) c4(255448) c5(255449, 255450, 255451, 255452, 255453, 255454, 255455) c6( ) c7(q, ci, s, r); #c1(GRB10) c2(NP_001001549) c3(growth factor receptor-bound protein 10 isoform b) c4(18670) c5(18671, 18672, 18673, 18674, 18675, 18676) c6( ) c7(bL, EW, bP, vJ, aW, ff, AH, LO, jx, aT, ml, amj, QF, m, ahl, en, bt, ahE, pb); #c1(GRB10) c2(NP_001001555) c3(growth factor receptor-bound protein 10 isoform c) c4( ) c5(18692, 18693, 18694, 18695, 18696, 18697, 18698, 18699, 18700, 18701, 18702, 18703, 18704, 18705, 18706, 18707, 18708, 18709, 18710, 18711) c6( ) c7(bL, EW, bP, vJ, aW, ff, AH, LO, jx, aT, ml, amj, QF, m, ahl, en, bt, ahE, pb); #c1(GRB10) c2(NP_005302) c3(growth factor receptor-bound protein 10 isoform a) c4(154779) c5(154780, 154781, 154782, 154783, 154784, 154785, 154786, 154787, 154788, 154789, 154790, 154791, 154792, 154793, 154794, 154795) c6( ) c7(bL, EW, bP, vJ, aW, ff, AH, LO, jx, aT, ml, amj, QF, m, ahl, en, bt, ahE, pb); #c1(GRB10) c2(XP_011513614) c3(growth factor receptor-bound protein 10 isoform X3) c4(305917, 305918, 305919, 305920) c5(305921, 305922, 305923, 305924, 305925, 305926, 305927, 305928, 305929, 305930, 305931, 305932, 305933, 305934, 305935, 305936) c6( ) c7(bL, EW, bP, vJ, aW, ff, AH, LO, jx, aT, ml, amj, QF, m, ahl, en, bt, ahE, pb); #c1(GRB10) c2(XP_011513625) c3(growth factor receptor-bound protein 10 isoform X4) c4( ) c5(305937, 305938, 305939, 305940, 305941) c6( ) c7(bL, EW, bP, vJ, aW, ff, AH, LO, jx, aT, ml, amj, QF, m, ahl, en, bt, ahE, pb); #c1(GRB14) c2(NP_001290351) c3(growth factor receptor-bound protein 14 isoform 2) c4(115234) c5(115235, 115236, 115237, 115238, 115239, 115240) c6( ) c7(bL, aW, hd, nB, aT); #c1(GRB14) c2(NP_004481) c3(growth factor receptor-bound protein 14 isoform 1) c4(146771) c5(146772, 146773, 146774, 146775, 146776, 146777) c6( ) c7(bL, aW, hd, nB, aT); #c1(GRB2) c2(NP_002077) c3(growth factor receptor-bound protein 2 isoform 1) c4(124300) c5(124301, 124302, 124303, 124304, 124305, 124306, 124307, 124308, 124309, 124310, 124311, 124312, 124313, 124314) c6( ) c7(bL, Mb, T, jx, aA, fd, ci, q, eA, ip, aT, hD, h, hn, dL, eL, af, jf, yo, Oz, iU, aW, r, aF, cm, t, op, ck, vA, B); #c1(GRB2) c2(NP_987102) c3(growth factor receptor-bound protein 2 isoform 2) c4(263417) c5(263418, 263419, 263420, 263421, 263422, 263423) c6( ) c7(bL, Mb, T, jx, aA, fd, ci, q, eA, ip, aT, hD, h, hn, dL, eL, af, jf, yo, Oz, iU, aW, r, af, cm, t, op, ck, vA, B); #c1(GRB7) c2(NP_001229371) c3(growth factor receptor-bound protein 7 isoform b) c4(84273, 84274) c5(84275, 84276, 84277, 84278, 84279, 84280, 84281, 84282) c6( ) c7(T, ht, aA, n, fr, ci, ad, av, aE, aT, yu, hu, iU, aW, aF, g, t, Ld, kP, cj, m, B); #c1(GRB7) c2(NP_001229372) c3(growth factor receptor-bound protein 7 isoform a) c4( ) c5(84283, 84284, 84285, 84286, 84287, 84288, 84289, 84290, 84291, 84292, 84293) c6( ) c7(T, ht, aA, n, fr, ci, ad, av, aE, aT, yu, hu, iU, aW, aF, g, t, Ld, kP, cj, m, B); #c1(GRB7) c2(XP_011522985) c3(growth factor receptor-bound protein 7 isoform X1) c4(317450) c5(317451, 317452, 317453, 317454, 317455, 317456, 317457, 317458, 317459, 317460) c6( ) c7(T, ht, aA, n, fr, ci, ad, av, aE, aT, yu, hu, iU, aW, aF, g, t, Ld, kP, cj, m, B); #c1(GREB1) c2(NP_683701) c3(protein GREB1 isoform c) c4(242433) c5(242434, 242435, 242436, 242437, 242438, 242439) c6( ) c7(dP, ae, T, kl, aW, aZ, aT, cz, m, t, bz, LS, ad); #c1(GREB1) c2(XP_005246249) c3(protein GREB1 isoform X1) c4(266983) c5(266984, 266985, 266986, 266987, 266988, 266989, 266990, 266991, 266992, 266993, 266994, 266995) c6( ) c7(dP, ae, T, kl, aW, aZ, aT, cz, m, t, bz, LS, ad); #c1(GREB1) c2(XP_011508725) c3(protein GREB1 isoform X2) c4(299341) c5(299342, 299343, 299344, 299345, 299346, 299347) c6( ) c7(dP, ae, T, kl, aW, aZ, aT, cz, m, t, bz, LS, ad); #c1(GREM1) c2(NP_001178252) c3(gremlin-1 isoform 2 precursor) c4(73301) c5(73302, 73303, 73304, 73305, 73306, 73307, 73308, 73309, 73310, 73311, 73312, 73313) c6(73314, 73315, 73316) c7(bL, T, dD, ea, fG, jh, aA, eq, ci, uE, rc, q, ad, dP, av, py, wG, afQ, oj, aT, h, Nc, ae, aa, s, are, ff, r, en, bvU, pb, bF, sS, arB, kP, eM, lC); #c1(GREM1) c2(NP_037504) c3(gremlin-1 isoform 1 precursor) c4(175879) c5(175880, 175881, 175882, 175883, 175884, 175885, 175886) c6(175887, 175888) c7(bL, T, dD, ea, fG, jh, aA, eq, ci, uE, rc, q, ad, dP, av, py, wG, afQ, oj, aT, h, Nc, ae, aa, s, are, ff, r, en, bvU, pb, bF, sS, arB, kP, eM, lC); #c1(GREM1) c2(XP_006725605) c3(gremlin-1 isoform X1) c4( ) c5(296912, 296913) c6( ) c7(bL, T, dD, ea, fG, jh, aA, eq, ci, uE, rc, q, ad, dP, av, py, wG, afQ, oj, aT, h, Nc, ae, aa, s, are, ff, r, en, bvU, pb, bF, sS, arB, kP, eM, lC); #c1(GREM2) c2(XP_005273283) c3(gremlin-2 isoform X2) c4(284432) c5(284433) c6( ) c7(V, bA, mr); #c1(GREM2) c2(XP_011542551) c3(gremlin-2 isoform X1) c4( ) c5(341102, 341103, 341104, 341105, 341106) c6( ) c7(V, bA, mr); #c1(GRHL1) c2(NP_937825) c3(grainyhead-like protein 1 homolog) c4(259210, 259211) c5(259212, 259213, 259214, 259215, 259216, 259217, 259218, 259219, 259220, 259221, 259222, 259223, 259224) c6( ) c7(B, bik, bE, bEY, r, bB, aq, cm, q); #c1(GRHL1) c2(XP_005246216) c3(grainyhead-like protein 1 homolog isoform X5) c4(266963) c5(266964, 266965, 266966, 266967, 266968, 266969, 266970, 266971, 266972, 266973, 266974) c6( ) c7(B, bik, bE, bEY, r, bB, aq, cm, q); #c1(GRHL2) c2(NP_079191) c3(grainyhead-like protein 2 homolog) c4(217416, 217417, 217418) c5(217419, 217420, 217421, 217422, 217423, 217424, 217425, 217426, 217427, 217428, 217429, 217430, 217431, 217432, 217433, 217434, 217435, 217436) c6( ) c7(LB, es, T, py, iA, lp, aW, fG, ff, aT, r, n, aq, q, en, bEZ, cM, B); #c1(GRHPR) c2(NP_036335) c3(glyoxylate reductase/hydroxypyruvate reductase) c4(173055, 173056, 173057) c5(173058, 173059, 173060, 173061, 173062, 173063, 173064, 173065, 173066, 173067, 173068, 173069) c6( ) c7(Tl, MB, ad); #c1(GRHPR) c2(XP_005251688) c3(glyoxylate reductase/hydroxypyruvate reductase isoform X1) c4( ) c5(271043, 271044, 271045, 271046, 271047, 271048) c6( ) c7(Tl, MB, ad); #c1(GRIA1) c2(NP_000818) c3(glutamate receptor 1 isoform 1 precursor) c4(16354, 16355) c5(16356, 16357, 16358, 16359, 16360, 16361, 16362, 16363, 16364, 16365, 16366) c6( ) c7(T, ha, bO, qy, bS, uu, ct, il, eA, xt, Ef, gM, lu, fS, h, lG, bK, he, af, d, aCt, bFc, aPf, cu, Z, bN, cT, ac, c, bFb, lw, bFa, cB, gO); #c1(GRIA1) c2(NP_001107655) c3(glutamate receptor 1 isoform 2 precursor) c4(42959, 42960) c5(42961, 42962, 42963, 42964, 42965, 42966, 42967, 42968, 42969, 42970, 42971, 42972, 42973, 42974, 42975, 42976) c6( ) c7(T, ha, bO, qy, bS, uu, ct, il, eA, xt, Ef, gM, lu, fS, h, lG, bK, he, af, d, aCt, bFc, aPf, cu, Z, bN, cT, ac, c, bFb, lw, bFa, cB, gO); #c1(GRIA1) c2(NP_001244948) c3(glutamate receptor 1 isoform 3 precursor) c4(92407) c5(92408, 92409, 92410, 92411, 92412, 92413) c6( ) c7(T, ha, bO, qy, bS, uu, ct, il, eA, xt, Ef, gM, lu, fS, h, lG, bK, he, af, d, aCt, bFc, aPf, cu, Z, bN, cT, ac, c, bFb, lw, bFa, cB, gO); #c1(GRIA1) c2(NP_001244950) c3(glutamate receptor 1 isoform 5) c4(92414) c5(92415, 92416, 92417, 92418, 92419, 92420, 92421) c6( ) c7(T, ha, bO, qy, bS, uu, ct, il, eA, xt, Ef, gM, lu, fS, h, lG, bK, he, af, d, aCt, bFc, aPf, cu, Z, bN, cT, ac, c, bFb, lw, bFa, cB, gO); #c1(GRIA1) c2(NP_001244951) c3(glutamate receptor 1 isoform 6) c4(92422) c5(92423, 92424, 92425, 92426, 92427, 92428) c6( ) c7(T, ha, bO, qy, bS, uu, ct, il, eA, xt, Ef, gM, lu, fS, h, lG, bk, he, af, d, aCt, bFc, aPf, cu, Z, bN, cT, ac, c, bFb, lw, bFa, cB, gO); #c1(GRIA2) c2(NP_000817) c3(glutamate receptor 2 isoform 1 precursor) c4(16342, 16343) c5(16344, 16345, 16346, 16347, 16348, 16349, 16350, 16351, 16352, 16353) c6( ) c7(ha, bO, aj, Vd, ci, nA, uu, il, abe, eA, aR, J, lu, M, bK, he, af, bFd, d, TQ, cu, g, ir, hq, ac, c, nG, bFb, cB); #c1(GRIA2) c2(NP_001077088) c3(glutamate receptor 2 isoform 2 precursor) c4(37726, 37727, 37728, 37729) c5(37730, 37731, 37732, 37733, 37734, 37735, 37736, 37737, 37738, 37739, 37740) c6( ) c7(ha, bO, aj, Vd, ci, nA, uu, il, abe, eA, aR, J, lu, M, bK, he, af, bFd, d, TQ, cu, g, ir, hq, ac, c, nG, bFb, cB); #c1(GRIA2) c2(NP_001077089) c3(glutamate receptor 2 isoform 3) c4( ) c5(37741, 37742, 37743, 37744, 37745, 37746, 37747, 37748, 37749, 37750, 37751) c6( ) c7(ha, bO, aj, Vd, ci, nA, uu, il, abe, eA, aR, J, lu, M, bK, he, af, bFd, d, TA, cu, g, ir, hq, ac, c, nG, bFb, cB); #c1(GRIA2) c2(XP_011530194) c3(glutamate receptor 2 isoform X1) c4(326085, 326086, 326087, 326088) c5(326089, 326090, 326091, 326092, 326093, 326094, 326095, 326096, 326097, 326098, 326099, 326100, 326101, 326102, 326103, 326104, 326105, 326106, 326107, 326108, 326109, 326110, 326111, 326112, 326113, 326114, 326115, 326116, 326117, 326118, 326119, 326120, 326121, 326122, 326123) c6( ) c7(ha, bO, aj, Vd, ci, nA, uu, il, abe, eA, aR, J, lu, M, bK, he, af, bFd, d, TQ, cu, g, ir, hq, ac, c, nG, bFb, cB); #c1(GRIA3) c2(NP_000819) c3(glutamate receptor 3 isoform 2 precursor) c4(16367, 16368) c5(16369, 16370, 16371, 16372, 16373, 16374, 16375, 16376, 16377, 16378, 16379, 16380, 16381, 16382, 16383, 16384, 16385, 16386, 16387, 16388, 16389, 16390) c6( ) c7(T, ha, jh, bS, uu, ct, nA, PJ, il, pK, Ef, aE, iX, bK, rE, af, nf, Z, bFf, ck, c, bFe, bFb, hh, gO, qc); #c1(GRIA3) c2(NP_001243672) c3(glutamate receptor 3 isoform 3 precursor) c4(91328, 91329) c5(91330, 91331, 91332, 91333, 91334, 91335, 91336, 91337, 91338, 91339, 91340, 91341, 91342, 91343, 91344, 91345) c6( ) c7(T, ha, jh, bS, uu, ct, nA, PJ, il, pK, Ef, aE, iX, bK, rE, af, nf, Z, bFf, ck, c, bFe, bFb, hh, gO, qc); #c1(GRIA3) c2(NP_015564) c3(glutamate receptor 3 isoform 1 precursor) c4(171890, 171891) c5(171892, 171893, 171894, 171895, 171896, 171897) c6( ) c7(T, ha, jh, bS, uu, ct, nA, PJ, il, pK, Ef, aE, iX, bK, rE, af, nf, Z, bFf, ck, c, bFe, bFb, hh, gO, qc); #c1(GRIA4) c2(NP_001070711) c3(glutamate receptor 4 isoform 2 precursor) c4(34790) c5(34791, 34792, 34793, 34794, 34795, 34796, 34797, 34798, 34799, 34800) c6( ) c7(d, c, HN, Be, bO, uu, bC, lB); #c1(GRIA4) c2(NP_001106283) c3(glutamate receptor 4 isoform 3 precursor) c4(42430) c5(42431, 42432, 42433, 42434, 42435, 42436, 42437, 42438, 42439, 42440, 42441, 42442, 42443, 42444, 42445, 42446, 42447, 42448, 42449, 42450) c6( ) c7(d, c, HN, Be, bO, uu, bC, lB); #c1(GRIA4) c2(XP_005271575) c3(glutamate receptor 4 isoform X2) c4( ) c5(283480) c6( ) c7(d, c, HN, Be, bO, uu, bC, lB); #c1(GRIA4) c2(XP_006718886) c3(glutamate receptor 4 isoform X1) c4(292033, 292034) c5(292035, 292036, 292037, 292038, 292039, 292040, 292041, 292042, 292043, 292044) c6( ) c7(d, c, HN, Be, bO, uu, bC, IB); #c1(GRID1) c2(NP_060021) c3(glutamate receptor ionotropic, delta-1 precursor) c4(193718, 193719) c5(193720, 193721, 193722, 193723, 193724, 193725, 193726, 193727, 193728, 193729, 193730, 193731) c6( ) c7(iH, lV, e, ha, bo, jy, bK, xw, bDu); #c1(GRID2) c2(NP_001273767) c3(glutamate receptor ionotropic, delta-2 isoform 2 precursor) c4(105821) c5(105822, 105823, 105824, 105825, 105826, 105827) c6( ) c7(cd, ky); #c1(GRID2) c2(NP_001501) c3(glutamate receptor ionotropic, delta-2 isoform 1 precursor) c4(118239) c5(118240, 118241, 118242, 118243, 118244, 118245) c6( ) c7(cd, ky); #c1(GRIK1) c2(NP_000821) c3(glutamate receptor ionotropic, kainate 1 isoform 1 precursor) c4(16391, 16392) c5(16393, 16394, 16395, 16396, 16397, 16398, 16399, 16400, 16401, 16402, 16403, 16404, 16405, 16406, 16407, 16408, 16409, 16410) c6( ) c7(Cq, rR, c, ha, HN, bO, aT, bk, PJ, il, f, fq, aR, cB); #c1(GRIK1) c2(NP_783300) c3(glutamate receptor ionotropic, kainate 1 isoform 2 precursor) c4(251580) c5(251581, 251582, 251583, 251584, 251585, 251586, 251587, 251588, 251589, 251590, 251591, 251592) c6( ) c7(Cq, rR, c, ha, HN, bO, aT, bk, PJ, il, f, fq, aR, cB); #c1(GRIK1) c2(XP_005260999) c3(glutamate receptor ionotropic, kainate 1 isoform X2) c4(277070, 277071) c5(277072, 277073, 277074, 277075, 277076, 277077) c6( ) c7(Cq, rR, c, ha, HN, bO, aT, bK, PJ, il, f, fq, aR, cB); #c1(GRIK1) c2(XP_005261000) c3(glutamate receptor ionotropic, kainate 1 isoform X3) c4(277078, 277079) c5(277080, 277081, 277082, 277083, 277084, 277085, 277086, 277087, 277088, 277089, 277090, 277091) c6( ) c7(Cq, rR, c, ha, HN, bO, aT, bK, PJ, il, f, fq, aR, cB); #c1(GRIK1) c2(XP_005261001) c3(glutamate receptor ionotropic, kainate 1 isoform X4) c4(277092) c5(277093, 277094, 277095, 277096, 277097, 277098) c6( ) c7(Cq, rR, c, ha, HN, bO, aT, bk, PJ, il, f, fq, aR, cB); #c1(GRIK1) c2(XP_006724054) c3(glutamate receptor ionotropic, kainate 1 isoform X1) c4(295669, 295670) c5(295671, 295672, 295673, 295674, 295675, 295676) c6( ) c7(Cq, rR, c, ha, HN, bO, aT, bk, PJ, il, f, fq, aR, cB); #c1(GRIK1) c2(XP_011527830) c3(glutamate receptor ionotropic, kainate 1 isoform X6) c4( ) c5(322770) c6( ) c7(Cq, rR, c, ha, HN, bO, aT, bk, PJ, il, f, fq, aR, cB); #c1(GRIK2) c2(XP_005267002) c3(glutamate receptor ionotropic, kainate 2 isoform X1) c4(280610) c5(280611, 280612, 280613, 280614, 280615, 280616, 280617, 280618, 280619) c6( ) c7(nD, oe, aw, aA, bS, ct, nA, il, Ef, x, bFg, lu, lG, hn, bK, hg, rE, ff, aF, en, Z, bN, hq, ek, rR, ac, c, cA, uJ, be, lB, gO, qc); #c1(GRIK2) c2(XP_005267003) c3(glutamate receptor ionotropic, kainate 2 isoform X4) c4(280620) c5(280621, 280622, 280623, 280624, 280625) c6( ) c7(nD, oe, aw, aA, bS, ct, nA, il, Ef, x, bFg, lu, lG, hn, bK, hg, rE, ff, aF, en, Z, bN, hq, eK, rR, ac, c, cA, uJ, be, lB, gO, qc); #c1(GRIK2) c2(XP_011534079) c3(glutamate receptor ionotropic, kainate 2 isoform X3) c4(330266, 330267) c5(330268, 330269, 330270, 330271, 330272) c6( ) c7(nD, oe, aw, aA, bS, ct, nA, il, Ef, x, bFg, lu, lG, hn, bK, hg, rE, ff, aF, en, Z, bN, hq, ek, rR, ac, c, cA, uJ, be, lB, gO, qc); #c1(GRIK2) c2(XP_011534080) c3(glutamate receptor ionotropic, kainate 2 isoform X2) c4(330273) c5(330274, 330275, 330276, 330277, 330278, 330279, 330280, 330281, 330282) c6( ) c7(nD, oe, aw, aA, bS, ct, nA, il, Ef, x, bFg, lu, lG, hn, bK, hg, rE, ff, aF, en, Z, bN, hq, ek, rR, ac, c, cA, uJ, be, lB, gO, qc); #c1(GRIK2) c2(XP_011534081) c3(glutamate receptor ionotropic, kainate 2 isoform X5) c4(330283, 330284) c5(330285, 330286, 330287, 330288, 330289, 330290) c6( ) c7(nD, oe, aw, aA, bS, ct, nA, il, Ef, x, bFg, lu, lG, hn, bK, hg, rE, ff, aF, en, Z, bN, hq, ek, rR, ac, c, cA, uJ, be, lB, gO, qc); #c1(GRIK2) c2(XP_011534082) c3(glutamate receptor ionotropic, kainate 2 isoform X6) c4( ) c5(330291, 330292, 330293, 330294, 330295, 330296) c6( ) c7(nD, oe, aw, aA, bS, ct, nA, il, Ef, x, bFg, lu, lG, hn, bK, hg, rE, ff, aF, en, Z, bN, hq, ek, rR, ac, c, cA, uJ, be, lB, gO, qc); #c1(GRIK2) c2(XP_011534083) c3(glutamate receptor ionotropic, kainate 2 isoform X7) c4( ) c5(330297, 330298, 330299, 330300, 330301, 330302) c6( ) c7(nD, oe, aw, aA, bS, ct, nA, il, Ef, x, bFg, lu, lG, hn, bK, hg, rE, ff, aF, en, Z, bN, hq, eK, rR, ac, c, cA, uJ, be, lB, gO, qc); #c1(GRIK4) c2(NP_001269399) c3(glutamate receptor ionotropic, kainate 4 isoform 1 precursor) c4(101584) c5(101585, 101586, 101587, 101588, 101589, 101590, 101591, 101592, 101593, 101594) c6( ) c7(ac, pN, Y, gl, bK, nA, he, jb); #c1(GRIK5) c2(NP_001287959) c3(glutamate receptor ionotropic, kainate 5 isoform 1 precursor) c4(113266, 113267) c5(113268, 113269, 113270, 113271, 113272, 113273) c6( ) c7(x, il, cA, ff, lG, uJ, bS, bK, ct, hg, hn, Z, qc, be, en, gO, bN, hq); #c1(GRIK5) c2(XP_005258878) c3(glutamate receptor ionotropic, kainate 5 isoform X1) c4(275844) c5(275845, 275846, 275847, 275848, 275849, 275850) c6( ) c7(x, il, cA, ff, lG, uJ, bS, bK, ct, hg, hn, Z, qc, be, en, gO, bN, hq); #c1(GRIK5) c2(XP_011525170) c3(glutamate receptor ionotropic, kainate 5 isoform X8) c4( ) c5(319612, 319613, 319614, 319615, 319616, 319617, 319618) c6( ) c7(x, il, cA, ff, lG, uJ, bS, bk, ct, hg, hn, Z, qc, be, en, gO, bN, hq); #c1(GRIN1) c2(NP_000823) c3(glutamate receptor ionotropic, NMDA 1 isoform GluN1-4a precursor) c4(16411) c5(16412, 16413, 16414, 16415, 16416, 16417) c6( ) c7(T, ha, v, il, MP, bFh, aT, gs, bk, Wm, an, af, Lg, cC, aW, cr, cu, FX, Hv, rR, bo, bFb, gD); #c1(GRIN1) c2(NP_001172019) c3(glutamate receptor ionotropic, NMDA 1 isoform GluN1-3b precursor) c4(72243) c5(72244, 72245, 72246, 72247, 72248, 72249) c6( ) c7(T, ha, v, il, MP, bFh, aT, gs, bK, Wm, an, af, Lg, cC, aW, cr, cu, FX, Hv, rR, bo, bFb, gD); #c1(GRIN1) c2(NP_001172020) c3(glutamate receptor ionotropic, NMDA 1 isoform GluN1-5b precursor) c4(72250) c5(72251, 72252, 72253, 72254, 72255, 72256) c6( ) c7(T, ha, v, il, MP, bFh, aT, gs, bK, Wm, an, af, Lg, cC, aW, cr, cu, FX, Hv, rR, bo, bFb, gD); #c1(GRIN1) c2(NP_015566) c3(glutamate receptor ionotropic, NMDA 1 isoform GluN1-1a precursor) c4(171898) c5(171899, 171900, 171901, 171902, 171903, 171904) c6( ) c7(T, ha, v, il, MP, bFh, aT, gs, bK, Wm, an, af, Lg, cC, aW, cr, cu, FX, Hv, rR, bo, bFb, gD); #c1(GRIN1) c2(NP_067544) c3(glutamate receptor ionotropic, NMDA 1 isoform GluN1-2a precursor) c4(208981) c5(208982, 208983, 208984, 208985, 208986, 208987) c6( ) c7(T, ha, v, il, MP, bFh, aT, gs, bK, Wm, an, af, Lg, cC, aW, cr, cu, FX, Hv, rR, bo, bFb, gD); #c1(GRIN1) c2(XP_005266128) c3(glutamate receptor ionotropic, NMDA 1 isoform X2) c4(279909, 279910) c5(279911, 279912, 279913, 279914, 279915, 279916, 279917, 279918, 279919, 279920, 279921, 279922) c6( ) c7(T, ha, v, il, MP, bFh, aT, gs, bK, Wm, an, af, Lg, cC, aW, cr, cu, FX, Hv, rR, bo, bFb, gD); #c1(GRIN1) c2(XP_005266129) c3(glutamate receptor ionotropic, NMDA 1 isoform X3) c4(279923) c5(279924, 279925, 279926, 279927, 279928, 279929) c6( ) c7(T, ha, v, il, MP, bFh, aT, gs, bK, Wm, an, af, Lg, cC, aW, cr, cu, FX, Hv, rR, bo, bFb, gD); #c1(GRIN2A) c2(NP_000824) c3(glutamate receptor ionotropic, NMDA 2A isoform 1 precursor) c4(16418) c5(16419, 16420, 16421, 16422, 16423, 16424, 16425, 16426, 16427, 16428, 16429, 16430, 16431, 16432) c6( ) c7(byR, bFk, T, aw, ha, al, lr, bO, bB, bS, ct, nA, C, bFj, il, q, hX, cF, hb, bFi, MP, nl, lG, cy, bK, aQM, he, rz, af, Lg, hZ, aWm, Y, aCt, nh, Z, akf, Hv, bN, aUn, gk, rR, ac, c, cA, bE, bFI, bFa, cB, gD); #c1(GRIN2A) c2(XP_011520763) c3(glutamate receptor ionotropic, NMDA 2A isoform X1) c4(315765, 315766) c5(315767, 315768, 315769, 315770, 315771, 315772, 315773, 315774, 315775, 315776, 315777, 315778) c6( ) c7(byR, bFk, T, aw, ha, al, lr, bO, bB, bS, ct, nA, C, bFj, il, q, hX, cF, hb, bFi, MP, nl, lG, cy, bK, aQM, he, rz, af, Lg, hZ, aWm, Y, aCt, nh, Z, akf, Hv, bN, aUn, gk, rR, ac, c, cA, bE, bFl, bFa, cB, gD); #c1(GRIN2B) c2(XP_011518932) c3(glutamate receptor ionotropic, NMDA 2B isoform X1) c4(313384) c5(313385, 313386, 313387, 313388, 313389, 313390) c6( ) c7(bM, cZ, T, iH, ha, al, lr, aA, bO, it, ex, bS, j, ct, nA, il, q, cd, kL, eW, HF, bFm, iX, MP, lG, cy, eV, bK, dd, bq, rz, an, rE, af, cC, VX, ff, HL, aF, en, sz, Z, FX, Hv, pH, gk, aqr, ac, c, rR, eM, bo, Gs, nw, yA, xh, cB, bFn, qc); #c1(GRIN2C) c2(NP_000826) c3(glutamate receptor ionotropic, NMDA 2C isoform 1 precursor) c4(16433) c5( ) c6( ) c7(bK); #c1(GRIN2C) c2(NP_001265482) c3(glutamate receptor ionotropic, NMDA 2C isoform 2 precursor) c4(98683) c5(98684, 98685, 98686, 98687, 98688, 98689) c6( ) c7(bK); #c1(GRIN2C) c2(XP_006721908) c3(glutamate receptor ionotropic, NMDA 2C isoform X4) c4( ) c5(294392, 294393, 294394, 294395, 294396, 294397) c6( ) c7(bK); #c1(GRIN2D) c2(NP_000827) c3(glutamate receptor ionotropic, NMDA 2D precursor) c4(16434) c5(16435, 16436, 16437, 16438, 16439, 16440, 16441) c6( ) c7(eW, bK, aR, HC); #c1(GRIN3A) c2(NP_597702) c3(glutamate receptor ionotropic, NMDA 3A precursor) c4(235051) c5(235052, 235053, 235054, 235055, 235056, 235057) c6( ) c7(od, Gv, ff, ht, l, oR, bK, en, lf, af, aR); #c1(GRIN3B) c2(NP_619635) c3(glutamate receptor ionotropic, NMDA 3B precursor) c4(236906) c5(236907, 236908, 236909, 236910, 236911, 236912) c6( ) c7(he, af, c); #c1(GRIN3B) c2(XP_011525971) c3(glutamate receptor ionotropic, NMDA 3B isoform X1) c4( ) c5(320353, 320354, 320355) c6( ) c7(he, af, c); #c1(GRINA) c2(NP_001009184) c3(protein lifeguard 1) c4(23531, 23532, 23533, 23534) c5(23535, 23536, 23537, 23538, 23539, 23540, 23541, 23542, 23543, 23544, 23545, 23546, 23547, 23548, 23549, 23550, 23551, 23552, 23553, 23554, 23555, 23556, 23557, 23558, 23559, 23560, 23561, 23562, 23563) c6( ) c7(rR, ha); #c1(GRIP1) c2(NP_066973) c3(glutamate receptor-interacting protein 1 isoform 1) c4(207922) c5(207923, 207924, 207925, 207926, 207927, 207928) c6( ) c7(aAW, XW, T, c, eM, aw, aT, ti, j); #c1(GRIP1) c2(XP_011536392) c3(glutamate receptor-interacting protein 1 isoform X2) c4(333693) c5( ) c6( ) c7(aAW, XW, T, c, eM, aw, aT, ti, j); #c1(GRIP1) c2(XP_011536395) c3(glutamate receptor-interacting protein 1 isoform X6) c4(333694) c5(333695, 333696, 333697, 333698, 333699, 333700, 333701, 333702, 333703, 333704, 333705, 333706) c6( ) c7(aAW, XW, T, c, eM, aw, aT, ti, j); #c1(GRK1) c2(NP_002920) c3(rhodopsin kinase) c4(133563, 133564) c5(133565, 133566, 133567, 133568, 133569, 133570, 133571, 133572, 133573, 133574, 133575, 133576) c6( ) c7(bFr, lR, bFo, Y, bgg, bFp, cd, bFq, bFs, bq, hh, nC, ayj); #c1(GRK4) c2(NP_001004056) c3(G protein-coupled receptor kinase 4 isoform beta) c4(20394) c5(20395, 20396, 20397, 20398, 20399, 20400) c6( ) c7(dD, ac, aW, vj, oe, uv, aw, aT, cz, j, K, eq, uC, ye, qa); #c1(GRK4) c2(NP_001004057) c3(G protein-coupled receptor kinase 4 isoform gamma) c4(20401) c5(20402, 20403, 20404, 20405, 20406, 20407) c6( ) c7(dD, ac, aW, vj, oe, uv, aw, aT, cz, j, K, eq, uC, ye, qa); #c1(GRK4) c2(NP_005298) c3(G protein-coupled receptor kinase 4 isoform delta) c4(154764) c5(154765, 154766, 154767, 154768, 154769, 154770) c6( ) c7(dD, ac, aW, vj, oe, uv, aw, aT, cz, j, K, eq, uC, ye, qa); #c1(GRK4) c2(NP_892027) c3(G protein-coupled receptor kinase 4 isoform alpha) c4(257660) c5(257661, 257662, 257663, 257664, 257665, 257666) c6( ) c7(dD, ac, aW, vj, oe, uv, aw, aT, cz, j, K, eq, uC, ye, qa); #c1(GRK4) c2(XP_011511759) c3(G protein-coupled receptor kinase 4 isoform X12) c4( ) c5(302456) c6( ) c7(dD, ac, aW, vj, oe, uv, aw, aT, cz, j, K, eq, uC, ye, qa); #c1(GRK5) c2(NP_005299) c3(G protein-coupled receptor kinase 5) c4(154771, 154772) c5(154773, 154774, 154775, 154776, 154777, 154778) c6( ) c7(bL, bM, T, cz, bB, j, C, cd, ad, cF, cc, an, ae, af, ak, hl, cm, W, bE, mx, ll, gZ); #c1(GRK5) c2(XP_005269764) c3(G protein-coupled receptor kinase 5 isoform X1) c4(282767) c5(282768, 282769, 282770, 282771, 282772, 282773, 282774) c6( ) c7(bL, bM, T, cz, bB, j, C, cd, ad, cF, cc, an, ae, af, ak, hl, cm, W, bE, mx, ll, gZ); #c1(GRK5) c2(XP_005269765) c3(G protein-coupled receptor kinase 5 isoform X2) c4(282775) c5(282776, 282777, 282778, 282779, 282780, 282781, 282782, 282783, 282784, 282785, 282786) c6( ) c7(bL, bM, T, cz, bB, j, C, cd, ad, cF, cc, an, ae, af, ak, hl, cm, W, bE, mx, ll, gZ); #c1(GRK6) c2(NP_001004105) c3(G protein-coupled receptor kinase 6 isoform C) c4( ) c5(20422, 20423, 20424, 20425, 20426) c6( ) c7(vj, Y, fd, an, y, cv); #c1(GRK6) c2(NP_001004106) c3(G protein-coupled receptor kinase 6 isoform A) c4(20427) c5(20428, 20429, 20430, 20431, 20432, 20433) c6( ) c7(vj, Y, fd, an, y, cv); #c1(GRK6) c2(NP_002073) c3(G protein-coupled receptor kinase 6 isoform B) c4(124263, (24264) c5(124265, 124266, 124267, 124268, 124269, 124270, 124271) c6( ) c7(vj, Y, fd, an, y, cv); #c1(GRK6) c2(XP_011532840) c3(G protein-coupled receptor kinase 6 isoform X4) c4( ) c5(328959, 328960) c6( ) c7(vj, Y, fd, an, y, cv); #c1(GRK7) c2(NP_631948) c3(G protein-coupled receptor kinase 7) c4(238409) c5(238410, 238411, 238412, 238413, 238414, 238415) c6( ) c7(bFo, bgg, bFs); #c1(GRM1) c2(NP_001264995) c3(metabotropic glutamate receptor 1 isoform beta precursor) c4(97676) c5(97677, 97678, 97679, 97680, 97681, 97682, 97683, 97684, 97685, 97686, 97687, 97688, 97689, 97690) c6( ) c7(bM, T, ha, bO, bS, fr, ct, ci, il, V, KB, aT, cB, aW, Y, eH, bFu, Ku, Z, eF, kP, ky, c, bFt, eo, aen, ao); #c1(GRM1) c2(XP_011534086) c3(metabotropic glutamate receptor 1 isoform X1) c4(330303) c5(330304, 330305, 330306, 330307, 330308, 330309, 330310, 330311, 330312, 330313) c6( ) c7(bM, T, ha, bO, bS, fr, ct, ci, il, V, KB, aT, cB, aW, Y, eH, bFu, Ku, Z, eF, kP, ky, c, bFt, eo, aen, ao); #c1(GRM2) c2(NP_000830) c3(metabotropic glutamate receptor 2 isoform a precursor) c4(16442) c5(16443, 16444, 16445, 16446, 16447, 16448) c6( ) c7(ha, bO, aj, bS, Vd, ct, il, abe, eA, J, lu, M, aaw, K, af, xa, d, TQ, gl, gu, Z, ac, c, cA, sv, bFb); #c1(GRM2) c2(XP_011531944) c3(metabotropic glutamate receptor 2 isoform X4) c4(327975) c5(327976, 327977, 327978, 327979, 327980, 327981) c6( ) c7(ha, bO, aj, bS, Vd, ct, il, abe, eA, J, lu, M, aaw, K, af, xa, d, TQ, gl, gu, Z, ac, c, cA, sv, bFb); #c1(GRM3) c2(NP_000831) c3(metabotropic glutamate receptor 3 precursor) c4(16449, 16450, 16451) c5(16452, 16453, 16454, 16455, 16456, 16457, 16458, 16459, 16460, 16461, 16462, 16463, 16464, 16465, 16466, 16467, 16468, 16469) c6( ) c7(T, iH, ha, bO, jh, mS, ti, bS, ct, il, eA, mW, bK, mY, mV, mU, Y, gl, Z, jb, mZ, FT, mX, bFf, ck, cA, sv, bFb, hh, jd, mT); #c1(GRM3) c2(XP_011514390) c3(metabotropic glutamate receptor 3 isoform X1) c4(307064, 307065, 307066) c5(307067, 307068, 307069, 307070, 307071, 307072, 307073, 307074, 307075, 307076, 307077) c6( ) c7(T, iH, ha, bO, jh, mS, ti, bS, ct, il, eA, mW, bK, mY, mV, mU, Y, gl, Z, jb, mZ, FT, mX, bFf, ck, cA, sv, bFb, hh, jd, mT); #c1(GRM3) c2(XP_011514392) c3(metabotropic glutamate receptor 3 isoform X3) c4(307078) c5(307079, 307080, 307081, 307082, 307083, 307084, 307085, 307086, 307087, 307088, 307089, 307090) c6( ) c7(T, iH, ha, bO, jh, mS, ti, bS, ct, il, eA, mW, bK, mY, mV, mU, Y, gl, Z, jb, mZ, FT, mX, bFf, ck, cA, sv, bFb, hh, jd, mT); #c1(GRM4) c2(NP_000832) c3(metabotropic glutamate receptor 4 isoform 1 precursor) c4(16470) c5(16471, 16472, 16473, 16474, 16475, 16476, 16477, 16478, 16479, 16480) c6( ) c7(jf, Cq, iH, eF, ha, HN, HC, cD, eH, iD, bK, hg, eo, il, V, X); #c1(GRM4) c2(NP_001243738) c3(metabotropic glutamate receptor 4 isoform 2) c4(91610) c5(91611, 91612, 91613, 91614, 91615, 91616) c6( ) c7(jf, Cq, iH, eF, ha, HN, HC, cD, eH, iD, bK, hg, eo, il, V, X); #c1(GRM4) c2(NP_001243741) c3(metabotropic glutamate receptor 4 isoform 5) c4(91617, 91618, 91619) c5(91620, 91621, 91622, 91623, 91624, 91625) c6( ) c7(jf, Cq, iH, eF, ha, HN, HC, cD, eH, iD, bK, hg, eo, il, V, X); #c1(GRM4) c2(NP_001243742) c3(metabotropic glutamate receptor 4 isoform 6) c4(91626) c5(91627, 91628, 91629, 91630, 91631, 91632, 91633, 91634, 91635, 91636, 91637, 91638, 91639) c6( ) c7(jf, Cq, iH, eF, ha, HN, HC, cD, eH, iD, bK, hg, eo, il, V, X); #c1(GRM4) c2(NP_001269776) c3(metabotropic glutamate receptor 4 isoform 8) c4( ) c5(102747, 102748, 102749, 102750, 102751) c6( ) c7(jf, Cq, iH, eF, ha, HN, HC, cD, eH, iD, bK, hg, eo, il, V, X); #c1(GRM5) c2(NP_000833) c3(metabotropic glutamate receptor 5 isoform b precursor) c4(16481) c5(16482, 16483, 16484, 16485, 16486, 16487, 16488, 16489, 16490, 16491) c6( ) c7(ex, bO, ti, bS, ct, il, V, uT, eV, bK, rz, he, af, cB, ak, eH, gl, cu, Ku, as, Z, eF, cm, bN, ok, ac, c, eo, lB, xg); #c1(GRM5) c2(XP_011541096) c3(metabotropic glutamate receptor 5 isoform X1) c4(339600) c5(339601, 339602, 339603, 339604, 339605, 339606, 339607, 339608, 339609, 339610) c6( ) c7(ex, bO, ti, bS, ct, il, V, uT, eV, bK, rz, he, af, cB, ak, eH, gl, cu, Ku, as, Z, eF, cm, bN, ok, ac, c, eo, lB, xg); #c1(GRM5) c2(XP_011541097) c3(metabotropic glutamate receptor 5 isoform X2) c4(339611) c5(339612, 339613, 339614, 339615, 339616, 339617, 339618) c6( ) c7(ex, bO, ti, bS, ct, il, V, uT, eV, bK, rz, he, af, cB, ak, eH, gl, cu, Ku, as, Z, eF, cm, bN, ok, ac, c, eo, lB, xg); #c1(GRM5) c2(NP_000834) c3(metabotropic glutamate receptor 6 precursor) c4(16492, 16493) c5(16494, 16495, 16496, 16497, 16498, 16499, 16500, 16501, 16502, 16503, 16504, 16505, 16506, 16507, 16508, 16509) c6( ) c7(cZ, aWm, lR, VR, bFv, Be, dd, ayj, hZ); #c1(GRM7) c2(NP_000835) c3(metabotropic glutamate receptor 7 isoform a precursor) c4(16510, 16511, 16512, 16513, 16514) c5(16515, 16516, 16517, 16518, 16519, 16520, 16521, 16522, 16523, 16524, 16525, 16526) c6( ) c7(cZ, gM, Z, lu, oe, bO, vL, Nc, ww, bK, dd, oi, he, lB, fq, na, hq); #c1(GRM7) c2(NP_870989) c3(metabotropic glutamate receptor 7 isoform b precursor) c4(255877) c5(255878, 255879, 255880, 255881, 255882, 255883, 255884, 255885, 255886, 255887, 255888, 255889, 255890, 255891, 255892, 255893, 255894, 255895, 255896, 255897, 255898, 255899, 255900, 255901, 255902, 255903, 255904, 255905) c6( ) c7(cZ, gM, Z, lu, oe, bO, vL, Nc, ww, bK, dd, oi, he, lB, fq, na, hq); #c1(GRM8) c2(XP_006716001) c3(metabotropic glutamate receptor 8 isoform X1) c4(290007, 290008, 290009) c5( ) c6( ) c7(aWm, aap, ft, ac, dY, rz, hZ, e, ha, aw, EQ, j, eo, Z, he, V, fq, eu, bN, hq); #c1(GRM8) c2(XP_011514394) c3(metabotropic glutamate receptor 8 isoform X2) c4(307091, 307092) c5(307093, 307094, 307095, 307096, 307097, 307098, 307099, 307100, 307101, 307102, 307103, 307104, 307105, 307106, 307107, 307108, 307109, 307110, 307111, 307112, 307113) c6( ) c7(aWm, aap, ft, ac, dY, rz, hZ, e, ha, aw, EQ, j, eo, Z, he, V, fq, eu, bN, hq); #c1(GRM8) c2(XP_011514395) c3(metabotropic glutamate receptor 8 isoform X3) c4( ) c5(307114, 307115, 307116, 307117, 307118, 307119, 307120, 307121, 307122, 307123, 307124, 307125, 307126) c6( ) c7(aWm, aap, ft, ac, dY, rz, hZ, e, ha, aw, EQ, j, eo, Z, he, V, fq, eu, bN, hq); #c1(GRN) c2(XP_005257310) c3(granulins isoform X1) c4(275021, 275022, 275023, 275024) c5(275025, 275026, 275027, 275028, 275029, 275030, 275031, 275032, 275033, 275034, 275035, 275036, 275037, 275038, 275039, 275040, 275041, 275042, 275043, 275044) c6( ) c7(T, ea, fd, Vd, ci, GK, xE, pX, K, H, eL, aW, alR, kt, cm, bw, bb, iA, bxS, U, cj, mk, m, act, hh, gD, bFB, dD, Ol, qN, ai, jz, Do, dL, aq, bFA, Lq, anx, it, B, L, bL, bFw, v, V, bFz, amg, hW, hb, WY, HC, aT, rO, an, ae, af, ak, azU, iU, Y, aaE, en, t, xw, rY, dS, ahN, c, bFx, bt, VB, hl, id, pY, jh, eq, ad, ot, sO, ds, KU, d, qt, ff, dq, iR, bFy, ek, es, dJ, aaG, ei, xg); #c1(GRP) c2(NP_001012530) c3(gastrin-releasing peptide isoform 2 preproprotein) c4(25066) c5(25067, 25068, 25069, 25070, 25071, 25072) c6( ) c7(gh, G, T, bM, jp, ht, aA, bB, it, hy, ww, fr, ci, C, q, aR, ad, eA, eW, av, ft, zw, aE, Db, gH, nv, pX, aT, Ze, K, hu, ae, dx, Yl, gj, d, tb, JI, aW, oV, ev, fJ, AQ, YC, r, ff, kp, aF, en, cl, g, t, cm, bz, ik, asE, es, eM, iA, dB, bE, AR, dJ, gX, hL, hR, m, y, bFC, X); #c1(GRP) c2(NP_001012531) c3(gastrin-releasing peptide isoform 3 preproprotein) c4(25073) c5(25074, 25075, 25076, 25077, 25078, 25079) c6( ) c7(gh, G, T, bM, jp, ht, aA, bB, it, hy, ww, fr, ci, C, q, aR, ad, eA, eW, av, ft, zw, aE, Db, gH, nv, pX, aT, Ze, K, hu, ae, dx, Yl, gj, d, tb, JI, aW, oV, ev, fJ, AQ, YC, r, ff, kp, aF, en, cl, g, t, cm, bz, ik, asE, es, eM, iA, dB, bE, AR, dJ, gX, hL, hR, m, y, bFC, X); #c1(GRP) c2(NP_002082) c3(gastrin-releasing peptide isoform 1 preproprotein) c4(124329) c5(124330, 124331, 124332, 124333, 124334, 124335) c6( ) c7(gh, G, T, bM, jp, ht, aA, bB, it, hy, ww, fr, ci, C, q, aR, ad, eA, eW, av, ft, zw, aE, Db, gH, nv, pX, aT, Ze, K, hu, ae, dx, Yl, gj, d, tb, JI, aW, oV, ev, fJ, AD, YC, r, ff, kp, aF, en, cl, g, t, cm, bz, ik, asE, es, eM, iA, dB, bE, AR, dJ, gX, hL, hR, m, y, bFC, X); #c1(GRPEL1) c2(NP_079472) c3(grpE protein homolog 1, mitochondrial precursor) c4(218223, 218224) c5(218225, 218226, 218227, 218228, 218229, 218230, 218231, 218232, 218233, 218234, 218235, 218236) c6( ) c7(h, Vp, Ea); #c1(GRPR) c2(NP_005305) c3(gastrin-releasing peptide receptor) c4(154835, 154836) c5(154837, 154838, 154839, 154840, 154841, 154842, 154843, 154844, 154845, 154846, 154847, 154848) c6( ) c7(T, aA, bB, ww, fr, ci, C, q, aR, ad, av, ft, ot, hw, ta, YB, dL, rz, ae, af, jf, tb, aW, ev, r, aF, t, cm, bz, ik, bN, es, eM, bE, AR, gX, Yz, m, X); #c1(GRSF1) c2(NP_001091947) c3(G-rich sequence factor 1 isoform 2) c4( ) c5(38796, 38797, 38798, 38799, 38800, 38801, 38802, 38803) c6( ) c7(ds); #c1(GRSF1) c2(NP_002083) c3(G-rich sequence factor 1 isoform 1) c4(124336, 124337) c5(124338, 124339, 124340, 124341, 124342, 124343, 124344, 124345, 124346, 124347, 124348, 124349, 124350, 124351, 124352, 124353, 124354, 124355) c6( ) c7(ds); #c1(GRSF1) c2(XP_005265738) c3(G-rich sequence factor 1 isoform X2) c4(279687, 279688, 279689, 279690) c5(279691, 279692, 279693, 279694, 279695, 279696, 279697, 279698, 279699, 279700, 279701, 279702, 279703, 279704) c6( ) c7(ds); #c1(GRXCR1) c2(NP_001073945) c3(glutaredoxin domain-containing cysteine-rich protein 1) c4(36642) c5(36643, 36644, 36645, 36646, 36647, 36648) c6( ) c7(bFD, Bh, aQi); #c1(GRXCR1) c2(XP_011511993) c3(glutaredoxin domain-containing cysteine-rich protein 1 isoform X1) c4( ) c5(302671) c6( ) c7(bFD, Bh, aQi); #c1(GRXCR2) c2(XP_011535976) c3(glutaredoxin domain-containing cysteine-rich protein 2 isoform X1) c4(333248) c5(333249, 333250, 333251, 333252, 333253, 333254) c6( ) c7(bFE); #c1(GSC) c2(NP_776248) c3(homeobox protein goosecoid) c4(251038) c5(251039, 251040, 251041, 251042, 251043, 251044, 251045, 251046, 251047, 251048, 251049) c6( ) c7(jf, T, A, d, iR, Kk, Az, ajn, jh, n, aNL, aq, K, bFG, eA, bFF, B); #c1(GSC2) c2(NP_005306) c3(homeobox protein goosecoid-2) c4(154849) c5(154850, 154851, 154852, 154853, 154854, 154855, 154856, 154857, 154858, 154859, 154860) c6(154861, 154862) c7(he, RX, xt, agd); #c1(GSDMA) c2(XP_006721895) c3(gasdermin-A isoform X1) c4(294385) c5(294386, 294387, 294388, 294389, 294390, 294391) c6( ) c7(bM, T, aF, A, aA); #c1(GSDMB) c2(XP_011523318) c3(gasdermin-B isoform X3) c4(317818, 317819) c5(317820, 317821, 317822, 317823, 317824, 317825, 317826, 317827, 317828, 317829, 317830, 317831) c6( ) c7(bM, T, iV, A, sp, mQ, aA, fe, aq, v, pM, y, CM, rk, JB); #c1(GSDMB) c2(XP_011523319) c3(gasdermin-B isoform X6) c4(3/7832) c5(317833, 317834, 317835, 317836, 317837, 317838, 317839, 317840, 317841, 317842) c6( ) c7(bM, T, iV, A, sp, ml, aA, fe, aq, v, pM, y, CM, rk, JB); #c1(GSDMB) c2(XP_011523320) c3(gasdermin-B isoform X4) c4(317843) c5(317844, 317845, 317846, 317847, 317848, 317849) c6( ) c7(bM, T, iV, A, sp, ml, aA, fe, aq, v, pM, y, CM, rk, JB); #c1(GSDMB) c2(XP_011523321) c3(gasdermin-B isoform X1) c4(317850) c5(317851, 317852, 317853, 317854, 317855, 317856) c6( ) c7(bM, T, iV, A, sp, ml, aA, fe, aq, v, pM, y, CM, rk, JB); #c1(GSDMB) c2(XP_011523322) c3(gasdermin-B isoform X2) c4(317857) c5(317858, 317859, 317860, 317861, 317862, 317863) c6( ) c7(bM, T, iV, A, sp, ml, aA, fe, aq, v, pM, y, cM, rk, JB); #c1(GSDMC) c2(NP_113603) c3(gasdermin-C) c4(221667) c5(221668, 221669, 221670, 221671, 221672, 221673) c6( ) c7(cT, eA, aA); #c1(GSDMD) c2(NP_079012) c3(gasdermin-D) c4(216553, 216554, 216555, 216556, 216557) c5(216558, 216559, 216560, 216561, 216562, 216563, 216564, 216565, 216566, 216567, 216568, 216569, 216570, 216571, 216572, 216573, 216574, 216575, 216576, 216577, 216578, 216579, 216580, 216581, 216582, 216583, 216584, 216585, 216586, 216587, 216588, 216589, 216590, 216591) c6( ) c7(aA); #c1(GSE1) c2(NP_001127945) c3(genetic suppressor element 1 isoform 2) c4( ) c5(49526, 49527, 49528, 49529, 49530, 49531, 49532, 49533, 49534, 49535, 49536, 49537, 49538) c6( ) c7(q, r); #c1(GSE1) c2(NP_055430) c3(genetic suppressor element 1 isoform 1) c4(181098) c5( ) c6( ) c7(q, r); #c1(GSE1) c2(XP_005255920) c3(genetic suppressor element 1 isoform X6) c4(273901) c5( ) c6( ) c7(q, r); #c1(GSG1L) c2(NP_001103233) c3(germ cell-specific gene 1-like protein isoform 1 precursor) c4(41858, 41859, 41860) c5(41861, 41862, 41863, 41864, 41865, 41866, 41867, 41868, 41869, 41870, 41871, 41872, 41873) c6( ) c7(af); #c1(GSG1L) c2(NP_653276) c3(germ cell-specific gene 1-like protein isoform 2) c4( ) c5(239478, 239479, 239480, 239481, 239482) c6( ) c7(af); #c1(GSG1L) c2(XP_011544051) c3(germ cell-specific gene 1-like protein isoform X1) c4(343243) c5(343244, 343245, 343246, 343247, 343248, 343249) c6( ) c7(af); #c1(GSK3A) c2(NP_063937) c3(glycogen synthase kinase-3 alpha) c4(202226, 202227) c5(202228, 202229, 202230, 202231, 202232, 202233, 202234, 202235, 202236, 202237, 202238, 202239, 202240, 202241, 202242, 202243) c6( ) c7(aC, aE, dq, aq, bK, ct, j, fr, ae, B, ad); #c1(GSK3B) c2(NP_001139628) c3(glycogen synthase kinase-3 beta isoform 2) c4(59571) c5(59572, 59573, 59574, 59575, 59576, 59577) c6( ) c7(T, aC, ea, bO, fd, bB, ci, aE, K, oi, yo, aW, g, eF, cm, mZ, bb, eM, U, dD, aw, aj, fr, ct, cd, Hx, zw, h, dL, bl, mY, mV, mU, aq, mX, cv, YE, cA, vL, at, E, B, mT, aA, mS, pd, q, J, eA, mj, aT, bK, jj, an, ae, af, jf, kl, s, iU, Y, ev, fO, r, lo, t, xw, ck, ac, gX, bt, aZ, atS, bS, eq, iJ, ad, hX, mW, sO, d, eH, dq, aF, Z, bz, LB, bF, D, bE, ZW, X); #c1(GSK3B) c2(NP_002084) c3(glycogen synthase kinase-3 beta isoform 1) c4(124356) c5(124357, 124358, 124359, 124360, 124361, 124362) c6( ) c7(T, aC, ea, bO, fd, bB, ci, aE, K, oi, yo, aW, g, eF, cm, mZ, bb, eM, U, dD, aw, aj, fr, ct, cd, Hx, zw, h, dL, bl, mY, mV, mU, aq, mX, cv, YE, cA, vL, at, E, B, mT, aA, mS, pd, q, J, eA, mj, aT, bK, jj, an, ae, af, jf, kl, s, iU, Y, ev, fO, r, lo, t, xw, ck, ac, gX, bt, aZ, atS, bS, eq, iJ, ad, hX, mW, sO, d, eH, dq, aF, Z, bz, LB, bF, D, bE, ZW, X); #c1(GSK3B) c2(XP_006713674) c3(glycogen synthase kinase-3 beta isoform X2) c4(287272) c5(287273, 287274, 287275, 287276, 287277, 287278) c6( ) c7(T, aC, ea, bO, fd, bB, ci, aE, K, oi, yo, aW, g, eF, cm, mZ, bb, eM, U, dD, aw, aj, fr, ct, cd, Hx, zw, h, dL, bl, mY, mV, mU, aq, mX, cv, YE, cA, vL, at, E, B, mT, aA, mS, pd, q, J, eA, mj, aT, bK, jj, an, ae, af, jf, kl, s, iU, Y, ev, fO, r, lo, t, xw, ck, ac, gX, bt, aZ, atS, bS, eq, iJ, ad, hX, mW, sO, d, eH, dq, aF, Z, bz, LB, bF, D, bE, ZW, X); #c1(GSN) c2(NP_000168) c3(gelsolin isoform a precursor) c4(3755, 3756, 3757, 3758) c5(3759, 3760, 3761, 3762, 3763, 3764, 3765, 3766) c6( ) c7(bM, T, aC, aXW, lP, if, l, bB, aKs, ci, Qp, wM, aE, aU, K, hu, H, eL, aa, aW, g, as, eM, U, m, R, pw, hh, dt, aw, ht, qN, zP, pn, cd, aOu, aMV, dL, Wm, A, fQ, aay, boD, GU, eR, asB, C, bFJ, q, eA, bFH, aT, ae, bA, jf, s, Y, la, r, en, f, nC, ck, aKp, WP, dy, gT, bt, ob, aal, ah, fu, ad, av, ib, Dq, bFl, acQ, d, ff, YZ, bz, bF, bE, kq, dX, y, iu); #c1(GSN) c2(NP_001121135) c3(gelsolin isoform f) c4( ) c5(45267) c6( ) c7(bM, T, aC, aXW, lP, if, l, bB, aKs, ci, Qp, wM, aE, aU, K, hu, H, eL, aa, aW, g, as, eM, U, m, R, pw, hh, dt, aw, ht, qN, zP, pn, cd, aOu, aMV, dL, Wm, A, fQ, aay, boD, GU, eR, asB, C, bFJ, q, eA, bFH, aT, ae, bA, jf, s, Y, la, r, en, f, nC, ck, aKp, WP, dy, gT, bt, ob, aal, ah, fu, ad, av, ib, Dq, bFl, acQ, d, ff, YZ, bz, bF, bE, kq, dX, y, iu); #c1(GSN) c2(NP_001244959) c3(gelsolin isoform e) c4(92451) c5(92452, 92453, 92454, 92455, 92456, 92457, 92458, 92459, 92460) c6( ) c7(bM, T, aC, aXW, lP, if, l, bB, aKs, ci, Qp, wM, aE, aU, K, hu, H, eL, aa, aW, g, as, eM, U, m, R, pw, hh, dt, aw, ht, qN, zP, pn, cd, aOu, aMV, dL, Wm, A, fQ, aay, boD, GU, eR, asB, C, bFJ, q, eA, bFH, aT, ae, bA, jf, s, Y, la, r, en, f, nC, ck, aKp, WP, dy, gT, bt, ob, aal, ah, fu, ad, av, ib, Dq, bFl, acQ, d, ff, YZ, bz, bF, bE, kq, dX, y, iu); #c1(GSN) c2(XP_005251997) c3(gelsolin isoform X6) c4( ) c5(271418, 271419) c6( ) c7(bM, T, aC, aXW, lP, if, l, bB, aKs, ci, Qp, wM, aE, aU, K, hu, H, eL, aa, aW, g, as, eM, U, m, R, pw, hh, dt, aw, ht, qN, zP, pn, cd, aOu, aMV, dL, Wm, A, fQ, aay, boD, GU, eR, asB, C, bFJ, q, eA, bFH, aT, ae, bA, jf, s, Y, la, r, en, f, nC, ck, aKp, WP, dy, gT, bt, ob, aal, ah, fu, ad, av, ib, Dq, bFl, acQ, d, ff, YZ, bz, bF, bE, kq, dX, y, iu); #c1(GSN) c2(XP_005252002) c3(gelsolin isoform X1) c4( ) c5(271420, 271421, 271422, 271423, 271424, 271425, 271426, 271427, 271428, 271429, 271430, 271431, 271432, 271433, 271434, 271435, 271436, 271437, 271438, 271439, 271440, 271441, 271442, 271443, 271444, 271445, 271446, 271447, 271448, 271449, 271450, 271451, 271452, 271453, 271454, 271455, 271456, 271457, 271458, 271459, 271460, 271461, 271462, 271463, 271464, 271465, 271466, 271467) c6( ) c7(bM, T, aC, aXW, lP, if, l, bB, aKs, ci, Qp, wM, aE, aU, K, hu, H, eL, aa, aW, g, as, eM, U, m, R, pw, hh, dt, aw, ht, qN, zP, pn, cd, aOu, aMV, dL, Wm, A, fQ, aay, boD, GU, eR, asB, C, bFJ, q, eA, bFH, aT, ae, bA, jf, s, Y, la, r, en, f, nC, ck, aKp, WP, dy, gT, bt, ob, aal, ah, fu, ad, av, ib, Dq, bFl, acQ, d, ff, YZ, bz, bF, bE, kq, dX, y, iu); #c1(GSN) c2(XP_0067 (7138) c3(gelsolin isoform X3) c4(290761) c5( ) c6( ) c7(bM, T, aC, aXW, lP, if, l, bB, aKs, ci, Qp, wM, aE, aU, K, hu, H, eL, aa, aW, g, as, eM, U, m, R, pw, hh, dt, aw, ht, qN, zP, pn, cd, aOu, aMV, dL, Wm, A, fQ, aay, boD, GU, eR, asB, C, bFJ, q, eA, bFH, aT, ae, bA, jf, s, Y, la, r, en, f, nC, ck, aKp, WP, dy, gT, bt, ob, aal, ah, fu, ad, av, ib, Dq, bFl, acQ, d, ff, YZ, bz, bF, bE, kq, dX, y, iu); #c1(GSN) c2(XP_006717142) c3(gelsolin isoform X2) c4(290762) c5(290763, 290764, 290765, 290766, 290767, 290768, 290769, 290770, 290771, 290772, 290773, 290774, 290775, 290776, 290777, 290778, 290779, 290780) c6( ) c7(bM, T, aC, aXW, lP, if, l, bB, aKs, ci, Qp, wM, aE, aU, K, hu, H, eL, aa, aW, g, as, eM, U, m, R, pw, hh, dt, aw, ht, qN, zP, pn, cd, aOu, aMV, dL, Wm, A, fQ, aay, boD, GU, eR, asB, C, bFJ, q, eA, bFH, aT, ae, bA, jf, s, Y, la, r, en, f, nC, ck, aKp, WP, dy, gT, bt, ob, aal, ah, fu, ad, av, ib, Dq, bFl, acQ, d, ff, YZ, bz, bF, bE, kq, dX, y, iu); #c1(GSPT1) c2(NP_001123478) c3(eukaryotic peptide chain release factor GTP-binding subunit ERF3A isoform 2) c4(47135) c5(47136, 47137, 47138, 47139, 47140, 47141, 47142) c6( ) c7(bM, T, co, iU, Qu, aW, hl, aT, aA, aj, aF, ci, hE, bt, bz); #c1(GSPT1) c2(NP_001123479) c3(eukaryotic peptide chain release factor GTP-binding subunit ERF3A isoform 3) c4( ) c5(47143, 47144, 47145, 47146, 47147) c6( ) c7(bM, T, co, iU, Qu, aW, hl, aT, aA, aj, aF, ci, hE, bt, bz); #c1(GSPT1) c2(NP_002085) c3(eukaryotic peptide chain release factor GTP-binding subunit ERF3A isoform 1) c4(124363, 124364) c5(124365, 124366, 124367, 124368, 124369, 124370, 124371) c6( ) c7(bM, T, co, iU, Qu, aW, hl, aT, aA, aj, aF, ci, hE, bt, bz); #c1(GSPT2) c2(NP_060564) c3(eukaryotic peptide chain release factor GTP-binding subunit ERF3B) c4(196593, 196594) c5(196595, 196596, 196597, 196598, 196599, 196600, 196601, 196602, 196603, 196604, 196605, 196606) c6( ) c7(ad); #c1(GSR) c2(NP_000628) c3(glutathione reductase, mitochondrial isoform 1 precursor) c4(14076, 14077) c5(14078, 14079, 14080, 14081, 14082, 14083, 14084, 14085, 14086, 14087, 14088, 14089) c6( ) c7(dD, ea, ol, oR, BH, v, j, Vd, oY, aET, eq, mj, F, ZX, oW, aT, dV, K, bq, af, bA, jf, aW, ff, S, en, g, cm, bz, ac, c, bo, hr, U, cj, E, oh, mg, at, pw, eg, aAN); #c1(GSR) c2(NP_001182031) c3(glutathione reductase, mitochondrial isoform 2 precursor) c4(74493) c5(74494, 74495, 74496, 74497, 74498, 74499) c6( ) c7(dD, ea, ol, oR, BH, v, j, Vd, oY, aET, eq, mj, F, ZX, oW, aT, dV, K, bq, af, bA, jf, aW, ff, S, en, g, cm, bz, ac, c, bo, hr, U, cj, E, oh, mg, at, pw, eg, aAN); #c1(GSR) c2(NP_00182032) c3(glutathione reductase, mitochondrial isoform 3 precursor) c4(74500) c5(74501, 74502, 74503, 74504, 74505, 74506) c6( ) c7(dD, ea, ol, oR, BH, v, j, Vd, oY, aET, eq, mj, F, ZX, oW, aT, dV, K, bq, af, bA, jf, aW, ff, S, en, g, cm, bz, ac, c, bo, hr, U, cj, E, oh, mg, at, pw, eg, aAN); #c1(GSR) c2(NP_001182033) c3(glutathione reductase, mitochondrial isoform 4 precursor) c4(74507) c5(74508, 74509, 74510, 74511, 74512, 74513) c6( ) c7(dD, ea, ol, oR, BH, v, j, Vd, oY, aET, eq, mj, F, ZX, oW, aT, dV, K, bq, af, bA, jf, aW, ff, S, en, g, cm, bz, ac, c, bo, hr, U, cj, E, oh, mg, at, pw, eg, aAN); #c1(GSS) c2(XP_011527098) c3(glutathione synthetase isoform X1) c4(321569, 321570, 321571) c5(321572, 321573, 321574, 321575, 321576, 321577, 321578, 321579, 321580, 321581, 321582, 321583, 321584, 321585, 321586, 321587, 321588, 321589) c6( ) c7(dD, nx, bFL, bBX, bB, eq, ci, zP, av, aaF, h, bxi, K, avn, zQ, bFK, S, g, zz, acp, dr, bF, axS, bE, U, at); #c1(GSTA1) c2(NP_665683) c3(glutathione S-transferase A1) c4(241652) c5(241653, 241654, 241655, 241656, 241657, 241658) c6( ) c7(bL, bM, T, eX, aA, cz, aj, j, q, J, ad, av, aT, eV, dL, K, ae, xa, eL, s, hG, aW, ev, ff, hl, r, hQ, aq, en, aot, g, t, aaD, cM, bz, bw, bF, gw, Lb, Yz, B); #c1(GSTA2) c2(XP_011512834) c3(glutathione S-transferase A2 isoform X1) c4(303880) c5(303881, 303882, 303883, 303884, 303885, 303886) c6( ) c7(Gl, ny, ib, aW, ht, cj, fQ, cy, aT, r, hy, K, Gu, Gw, bz, ad); #c1(GSTA4) c2(XP_005249092) c3(glutathione S-transferase A4 isoform X1) c4(268985, 268986, 268987) c5(268988, 268989, 268990, 268991, 268992, 268993, 268994, 268995, 268996, 268997) c6( ) c7(bL, bM, aC, dD, ea, aj, v, eq, ci, Qo, J, av, ib, afQ, dL, K, an, bq, af, ny, ff, r, aq, en, bz, eM, hk, at, B); #c1(GSTA4) c2(XP_011512837) c3(glutathione S-transferase A4 isoform X2) c4( ) c5(303887, 303888, 303889, 303890, 303891, 303892, 303893, 303894, 303895, 303896, 303897, 303898, 303899, 303900, 303901, 303902) c6( ) c7(bL, bM, aC, dD, ea, aj, v, eq, ci, Qo, J, av, ib, afQ, dL, K, an, bq, af, ny, ff, r, aq, en, bz, eM, hk, at, B); #c1(GSTA5) c2(NP_714543) c3(glutathione S-transferase A5) c4(246554) c5(246555, 246556, 246557, 246558, 246559, 246560, 246561, 246562, 246563, 246564) c6( ) c7(cz); #c1(GSTCD) c2(NP_001026890) c3(glutathione S-transferase C-terminal domain-containing protein isoform 1) c4(29861, 29862, 29863, 29864) c5(29865, 29866, 29867, 29868, 29869, 29870, 29871, 29872, 29873, 29874, 29875, 29876, 29877, 29878, 29879, 29880, 29881, 29882, 29883, 29884, 29885, 29886, 29887, 29888, 29889, 29890) c6( ) c7(cz, at, Dp, ad, hm, aa); #c1(GSTCD) c2(NP_079027) c3(glutathione S-transferase C-terminal domain-containing protein isoform 2) c4(216646) c5(216647, 216648, 216649, 216650, 216651, 216652, 216653, 216654, 216655, 216656, 216657, 216658) c6( ) c7(cz, at, Dp, ad, hm, aa); #c1(GSTK1) c2(NP_001137151) c3(glutathione S-transferase kappa 1 isoform b) c4(55032) c5(55033, 55034, 55035, 55036, 55037, 55038) c6( ) c7(bM, T, pf, ea, if, e, al, fd, bB, qj, ci, VV, x, aE, u, dV, K, bU, hu, hH, dF, eL, azh, eG, dH, Ow, aW, JI, iE, g, as, eE, eF, cm, bw, yd, xK, eM, nB, cj, dW, aZo, k, m, cE, dD, gU, aWG, ht, aw, n, aj, fr, qN, pM, ai, br, b, ft, aMV, h, eV, hn, dL, vd, hB, he, dx, A, asp, sp, CM, AQ, bed, ge, fe, aq, bFN, pm, dw, kk, Ld, iO, W, bP, asl, dB, uw, at, bFO, it, B, ayV, bL, eR, aZy, Dh, yk, jx, eX, aA, cz, fF, v, j, Qo, q, J, eA, hb, aT, ae, af, bA, qw, avV, s, iU, Y, Lf, afS, r, lo, en, bu, t, iK, pb, zJ, ck, sS, nM, ac, c, ig, Nk, gX, wR, bt, rA, ao, hS, gx, id, akd, HM, jh, bs, lD, oZ, oH, Gn, eq, hF, ad, hX, dP, av, l, hd, oW, aNp, ji, d, lU, bFM, ff, eH, aZw, cr, eZ, Ej, aF, xH, S, bFP, iV, bz, Ga, qa, eK, es, bE, gZ, i, hE, iu, y, pC); #c1(GSTK1) c2(NP_001137152) c3(glutathione S-transferase kappa 1 isoform c) c4(55039) c5(55040, 55041, 55042, 55043, 55044, 55045) c6( ) c7(bM, T, pf, ea, if, e, al, fd, bB, qj, ci, VV, x, aE, u, dV, K, bU, hu, hH, dF, eL, azh, eG, dH, Ow, aW, JI, iE, g, as, eE, eF, cm, bw, yd, xK, eM, nB, cj, dW, aZo, k, m, cE, dD, gU, aWG, ht, aw, n, aj, fr, qN, pM, ai, br, b, ft, aMV, h, eV, hn, dL, vd, hB, he, dx, A, asp, sp, CM, AQ, bed, ge, fe, aq, bFN, pm, dw, kk, Ld, iO, W, bP, asl, dB, uw, at, bFO, it, B, ayV, bL, eR, aZy, Dh, yk, jx, eX, aA, cz, fF, v, j, Qo, q, J, eA, hb, aT, ae, af, bA, qw, avV, s, iU, Y, Lf, afS, r, lo, en, bu, t, iK, pb, zJ, ck, sS, nM, ac, c, ig, Nk, gX, wR, bt, rA, ao, hS, gx, id, akd, HM, jh, bs, lD, oZ, oH, Gn, eq, hF, ad, hX, dP, av, l, hd, oW, aNp, ji, d, lU, bFM, ff, eH, aZw, cr, eZ, Ej, aF, xH, S, bFP, iV, bz, Ga, qa, eK, es, bE, gZ, i, hE, iu, y, pC); #c1(GSTK1) c2(NP_001137153) c3(glutathione S-transferase kappa 1 isoform d) c4(55046) c5(55047, 55048, 55049, 55050, 55051, 55052) c6( ) c7(bM, T, pf, ea, if, e, al, fd, bB, qj, ci, VV, x, aE, u, dV, K, bU, hu, hH, dF, eL, azh, eG, dH, Ow, aW, JI, iE, g, as, eE, eF, cm, bw, yd, xK, eM, nB, cj, dW, aZo, k, m, cE, dD, gU, aWG, ht, aw, n, aj, fr, qN, pM, ai, br, b, ft, aMV, h, eV, hn, dL, vd, hB, he, dx, A, asp, sp, CM, AQ, bed, ge, fe, aq, bFN, pm, dw, kk, Ld, iO, W, bP, asl, dB, uw, at, bFO, it, B, ayV, bL, eR, aZy, Dh, yk, jx, eX, aA, cz, fF, v, j, Qo, q, J, eA, hb, aT, ae, af, bA, qw, avV, s, iU, Y, Lf, afS, r, lo, en, bu, t, iK, pb, zJ, ck, sS, nM, ac, c, ig, Nk, gX, wR, bt, rA, ao, hS, gx, id, akd, HM, jh, bs, lD, oZ, oH, Gn, eq, hF, ad, hX, dP, av, l, hd, oW, aNp, ji, d, lU, bFM, ff, eH, aZw, cr, eZ, Ej, aF, xH, S, bFP, iV, bz, Ga, qa, eK, es, bE, gZ, i, hE, iu, y, pC); #c1(GSTK1) c2(NP_057001) c3(glutathione S-transferase kappa 1 isoform a) c4(188409, 188410) c5(188411, 188412, 188413, 188414, 188415, 188416, 188417, 188418, 188419, 188420, 188421, 188422) c6( ) c7(bM, T, pf, ea, if, e, al, fd, bB, qj, ci, VV, x, aE, u, dV, K, bU, hu, hH, dF, eL, azh, eG, dH, Ow, aW, JI, iE, g, as, eE, eF, cm, bw, yd, xK, eM, nB, cj, dW, aZo, k, m, cE, dD, gU, aWG, ht, aw, n, aj, fr, qN, pM, ai, br, b, ft, aMV, h, eV, hn, dL, vd, hB, he, dx, A, asp, sp, CM, AQ, bed, ge, fe, aq, bFN, pm, dw, kk, Ld, iO, W, bP, asl, dB, uw, at, bFO, it, B, ayV, bL, eR, aZy, Dh, yk, jx, eX, aA, cz, fF, v, j, Qo, q, J, eA, hb, aT, ae, af, bA, qw, avV, s, iU, Y, Lf, afS, r, lo, en, bu, t, iK, pb, zJ, ck, sS, nM, ac, c, ig, Nk, gX, wR, bt, rA, ao, hS, gx, id, akd, HM, jh, bs, lD, oZ, oH, Gn, eq, hF, ad, hX, dP, av, l, hd, oW, aNp, ji, d, lU, bFM, ff, eH, aZw, cr, eZ, Ej, aF, xH, S, bFP, iV, bz, Ga, qa, eK, es, bE, gZ, i, hE, iu, y, pC); #c1(GSTM3) c2(XP_011539598) c3(glutathione S-transferase Mu 3 isoform X1) c4( ) c5(337824) c6( ) c7(GI, G, FW, atT, T, dD, bM, id, if, aWG, ht, iW, aA, cz, hy, Gn, j, eq, Qo, qN, fw, q, ad, eA, ip, av, co, ib, afd, aT, io, Gw, dL, K, hu, hB, an, ae, af, bA, ny, B, BN, aW, Y, ff, ml, r, GA, aq, Ej, en, g, dw, t, as, bz, bmH, eF, es, fT, sS, dB, hk, Gs, cj, NI, at, Gu, od, bD, rA, ne, ao); #c1(GSTO1) c2(NP_001177931) c3(glutathione S-transferase omega-1 isoform 2) c4(73194, 73195) c5(73196, 73197, 73198, 73199, 73200, 73201, 73202, 73203, 73204, 73205, 73206, 73207) c6( ) c7(bM, T, dD, fM, id, AS, ea, e, eq, ci, ad, hX, Vp, hd, jz, dY, ec, lg, Db, Bj, aT, dL, K, lQ, hu, an, eL, xa, af, eu, aa, zQ, je, aW, LZ, r, aq, gW, wA, cM, ck, es, rR, iM, ac, anT, c, nB, U, Be, at, vn, cS, k, ei, NB, Pk, it, B, us); #c1(GSTO1) c2(NP_001177932) c3(glutathione S-transferase omega-1 isoform 3) c4( ) c5(73208, 73209, 73210, 73211, 73212, 73213, 73214, 73215, 73216, 73217) c6( ) c7(bM, T, dD, fM, id, AS, ea, e, eq, ci, ad, hX, Vp, hd, jz, dY, ec, lg, Db, Bj, aT, dL, K, lQ, hu, an, eL, xa, af, eu, aa, zQ, je, aW, LZ, r, aq, gW, wA, cM, ck, es, rR, iM, ac, anT, c, nB, U, Be, at, vn, cS, k, ei, NB, Pk, it, B, us); #c1(GSTO1) c2(NP_004823) c3(glutathione S-transferase omega-1 isoform 1) c4(150068, 150069, 150070) c5(150071, 150072, 150073, 150074, 150075, 150076, 150077, 150078) c6( ) c7(bM, T, dD, fM, id, AS, ea, e, eq, ci, ad, hX, Vp, hd, jz, dY, ec, lg, Db, Bj, aT, dL, K, lQ, hu, an, eL, xa, af, eu, aa, zQ, je, aW, LZ, r, aq, gW, wA, cM, ck, es, rR, iM, ac, anT, c, nB, U, Be, at, vn, cS, k, ei, NB, Pk, it, B, us); #c1(GSTO2) c2(NP_001177942) c3(glutathione S-transferase omega-2 isoform 2) c4(73218) c5(73219, 73220, 73221, 73222, 73223, 73224) c6( ) c7(bM, T, id, e, aA, ci, Qo, aT, dL, K, lQ, an, eL, af, zQ, aW, r, aq, gW, t, es, rR, c, at, cS, m, k, NB, Pk, B); #c1(GSTO2) c2(NP_001177943) c3(glutathione S-transferase omega-2 isoform 3) c4( ) c5(73225, 73226, 73227, 73228, 73229) c6( ) c7(bM, T, id, e, aA, ci, Qo, aT, dL, K, lQ, an, eL, af, zQ, aW, r, aq, gW, t, es, rR, c, at, cS, m, k, NB, Pk, B); #c1(GSTO2) c2(XP_011537572) c3(glutathione S-transferase omega-2 isoform X1) c4(335159, 335160) c5(335161, 335162, 335163, 335164, 335165, 335166, 335167, 335168, 335169, 335170, 335171) c6( ) c7(bM, T, id, e, aA, ci, Qo, aT, dL, K, lQ, an, eL, af, zQ, aW, r, aq, gW, t, es, rR, c, at, cS, m, k, NB, Pk, B); #c1(GSTO2) c2(XP_011537574) c3(glutathione S-transferase omega-2 isoform X3) c4(335/72) c5(335173, 335174, 335175, 335176, 335177, 335178) c6( ) c7(bM, T, id, e, aA, ci, Qo, aT, dL, K, lQ, an, eL, af, zQ, aW, r, aq, gW, t, es, rR, c, at, cS, m, k, NB, Pk, B); #c1(GSTO2) c2(XP_011537575) c3(glutathione S-transferase omega-2 isoform X4) c4(335179) c5(335180, 335181, 335182, 335183, 335184, 335185) c6( ) c7(bM, T, id, e, aA, ci, Qo, aT, dL, K, lQ, an, eL, af, zQ, aW, r, aq, gW, t, es, rR, c, at, cS, m, k, NB, Pk, B); #c1(GSTP1) c2(NP_000843) c3(glutathione S-transferase P) c4(16527) c5(16528, 16529, 16530, 16531, 16532, 16533) c6( ) c7(bM, G, T, aC, fR, pf, atj, if, al, hE, fd, bB, c, qj, ci, Jx, fw, sD, VV, aR, x, aEQ, Na, Gt, dY, aE, fS, Qk, dV, K, hu, dF, xa, bc, hm, eL, li, yo, zQ, dH, iE, aW, Ls, az, hl, mQ, LZ, kt, iG, fc, aB, g, as, eE, eF, uC, cm, bw, y, fT, xK, eM, nB, ii, nF, Jp, iA, iT, cj, uo, sv, bFS, dW, k, m, eg, cE, iu, FW, gU, dD, sQ, OI, aWG, ht, NT, qy, aw, n, BH, aj, oY, fr, qN, pM, rP, ai, awq, co, ft, jz, Bj, gd, eV, Gw, dL, hB, Um, pl, bFQ, Ck, PT, RT, sp, AQ, aRa, ge, ks, aq, kp, mo, wZ, dw, cM, Ld, Zj, cv, ar, bbz, ok, W, vD, asl, dB, hk, eD, Qu, hL, hR, E, ut, at, aiS, fL, YH, lB, sZ, B, aAE, it, ayV, bL, pF, fM, aCw, jp, Dh, eR, oe, eX, aA, cz, fF, hy, v, j, azF, bX, Qo, Eh, q, J, o, zn, eA, XX, ga, mP, nv, afQ, aT, cy, rO, Nc, adV, an, ae, af, qw, avV, s, iU, Y, Lf, r, akY, en, bu, cl, t, f, ik, bN, ck, sS, Dy, c, pZ, alt, gX, cS, dy, Yz, fD, bt, rA, bFR, ao, blu, vi, hS, gV, Gl, am, id, akd, ob, Mb, iW, uG, jh, oZ, Fb, Jz, jE, fu, uu, ah, Gn, hF, eq, Fc, ad, kL, hX, dP, av, iC, hd, afm, yt, Db, NS, lC, oW, io, HD, lQ, Od, aNR, bsn, NP, d, BN, BO, hG, qt, bFM, ff, eH, eZ, gl, aF, aot, gW, aGj, Sm, bz, iV, iq, TA, es, gk, bF, bE, z, Ny, X, azT, Lb, bkh, Gu, oQ, Pk, aax, ne, rk, pC); #c1(GSTT1) c2(NP_000844) c3(glutathione S-transferase theta-1 isoform a) c4(16534, 16535) c5(16536, 16537, 16538, 16539, 16540, 16541, 16542) c6( ) c7(T, ea, fd, ci, aY, aE, hu, hH, az, Qv, lk, fT, iA, nB, cj, k, eg, cE, Uc, ht, aj, br, Gw, mJ, pl, ge, fe, pm, bbz, dB, bFW, bFV, abW, aAE, jv, aA, GP, cz, bX, Eh, q, akU, jj, sR, sX, eu, bA, avV, aMA, lo, cl, pb, ac, ig, mG, bt, rA, ao, gV, iW, jh, KO, oZ, uu, fu, asl, Gn, ad, hX, iC, yt, oW, lQ, NP, ZE, BO, cW, bFM, ff, gK, Ce, bpA, EZ, iV, Sm, bz, iq, oQ, i, Pk, iu, ne, rk, bM, if, e, bB, cL, bh, sD, Tz, ip, x, Na, bFU, ja, bc, dH, JI, fc, hl, kt, NR, as, eE, eF, bw, qJ, ii, vy, dD, gU, n, oY, awq, b, Bj, gd, aMj, eV, hn, aOX, A, fJ, AL, bpx, cv, abV, hL, at, ut, B, pF, aCw, eX, fF, v, Qo, V, eJ, ae, jf, hM, HL, bu, t, ck, gX, Yz, vi, HQ, eq, Fc, hd, Db, Hk, hD, d, hG, gW, Z, bF, pP, y, pC, atT, aC, fR, atj, al, lj, qj, K, wt, dF, xa, eL, aAX, mQ, wF, LZ, hN, aB, g, uC, ou, uo, dW, bFS, m, FW, sQ, sw, Ol, aw, fN, qy, BH, pM, ft, vd, wb, FZ, ks, Pp, wZ, cM, Ld, Zj, ar, W, aiS, it, ayV, fM, AL, jx, azF, j, qf, eA, J, o, XX, hb, Nc, kl, Y, r, en, iK, sn, auo, Dy, cS, dy, fD, bFR, gx, Gl, id, akd, bs, iG, ah, hF, dP, fU, io, aOh, lU, BN, eH, Sn, cr, qa, TA, Zi, bkh, Gu, hE, G, pf, qx, eb, fw, aR, aEQ, dY, il, hm, ayT, zQ, aW, Rr, OR, yd, eM, iT, Jp, uJ, pw, aWG, fr, qN, ai, FY, co, cc, h, dL, hB, Gc, aq, dw, Qy, vD, hk, uw, hR, uz, YH, bFT, bL, eR, yk, hy, ga, uT, aoA, afQ, aT, vH, bK, an, af, qw, s, ND, iU, hf, f, sS, nM, pZ, aJt, hS, qb, aD, ha, lD, jE, kL, av, F, NS, Od, fb, eZ, gl, Ej, aF, aot, bmH, ek, es, apM, bE, z, X); #c1(GSTT1) c2(NP_001280736) c3(glutathione S-transferase theta-1 isoform b) c4(111580) c5(111581) c6( ) c7(T, ea, fd, ci, aY, aE, hu, hH, az, Qv, lk, fT, iA, nB, cj, k, eg, cE, Uc, ht, aj, br, Gw, mJ, pl, ge, fe, pm, bbz, dB, bFW, bFV, abW, aAE, jv, aA, GP, cz, bX, Eh, q, akU, jj, sR, sX, eu, bA, avV, aMA, lo, cl, pb, ac, ig, mG, bt, rA, ao, gV, iW, jh, KO, oZ, uu, fu, asl, Gn, ad, hX, iC, yt, oW, lQ, NP, ZE, BO, cW, bFM, ff, gK, Ce, bpA, EZ, iV, Sm, bz, iq, oQ, i, Pk, iu, ne, rk, bM, if, e, bB, cL, bh, sD, Tz, ip, x, Na, bFU, ja, bc, dH, JI, fc, hl, kt, NR, as, eE, eF, bw, qJ, ii, vy, dD, gU, n, oY, awq, b, Bj, gd, aMj, eV, hn, aOX, A, fJ, AQ, bpx, cv, abV, hL, at, ut, B, pF, aCw, eX, fF, v, Qo, V, eJ, ae, jf, hM, HL, bu, t, ck, gX, Yz, vi, HQ, eq, Fc, hd, Db, Hk, hD, d, hG, gW, Z, bF, pP, y, pC, atT, aC, fR, atj, al, lj, qj, K, wt, dF, xa, eL, aAX, mQ, wF, LZ, hN, aB, g, uC, ou, uo, dW, bFS, m, FW, sQ, sw, OI, aw, fN, qy, BH, pM, ft, vd, wb, FZ, ks, Pp, wZ, cM, Ld, Zj, ar, W, aiS, it, ayV, fM, AL, jx, azF, j, qf, eA, J, o, XX, hb, Nc, kl, Y, r, en, iK, sn, auo, Dy, cS, dy, fD, bFR, gx, Gl, id, akd, bs, iG, ah, hF, dP, fU, io, aOh, lU, BN, eH, Sn, cr, qa, TA, Zi, bkh, Gu, hE, G, pf, qx, eb, fw, aR, aEQ, dY, il, hm, ayT, zQ, aW, Rr, OR, yd, eM, iT, Jp, uJ, pw, aWG, fr, qN, ai, FY, co, cc, h, dL, hB, Gc, aq, dw, Qy, vD, hk, uw, hR, uz, YH, bFT, bL, eR, yk, hy, ga, uT, aoA, afQ, aT, vH, bK, an, af, qw, s, ND, iU, hf, f, sS, nM, pZ, aJt, hS, qb, aD, ha, lD, jE, kL, av, F, NS, Od, fb, eZ, gl, Ej, aF, aot, bmH, eK, es, apM, bE, z, X); #c1(GSTT1) c2(NP_001280741) c3(glutathione S-transferase theta-1 isoform c) c4( ) c5(111582, 111583, 111584, 111585, 111586, 111587, 111588, 111589, 111590, 111591, 111592, 111593) c6( ) c7(T, ea, fd, ci, aY, aE, hu, hH, az, Qv, lk, fT, iA, nB, cj, k, eg, cE, Uc, ht, aj, br, Gw, mJ, pl, ge, fe, pm, bbz, dB, bFW, bFV, abW, aAE, jv, aA, GP, cz, bX, Eh, q, akU, jj, sR, sX, eu, bA, avV, aMA, lo, cl, pb, ac, ig, mG, bt, rA, ao, gV, iW, jh, KO, oZ, uu, fu, asl, Gn, ad, hX, iC, yt, oW, lQ, NP, ZE, BO, cW, bFM, ff, gK, Ce, bpA, EZ, iV, Sm, bz, iq, oQ, i, Pk, iu, ne, rk, bM, if, e, bB, cL, bh, sD, Tz, ip, x, Na, bFU, ja, bc, dH, JI, fc, hl, kt, NR, as, eE, eF, bw, qJ, ii, vy, dD, gU, n, oY, awq, b, Bj, gd, aMj, eV, hn, aOX, A, fJ, AQ, bpx, cv, abV, hL, at, ut, B, pF, aCw, eX, fF, v, Qo, V, eJ, ae, jf, hM, HL, bu, t, ck, gX, Yz, vi, HQ, eq, Fc, hd, Db, Hk, hD, d, hG, gW, Z, bF, pP, y, pC, atT, aC, fR, atj, al, lj, qj, K, wt, dF, xa, eL, aAX, mQ, wF, LZ, hN, aB, g, uC, ou, uo, dW, bFS, m, FW, sQ, sw, OI, aw, fN, qy, BH, pM, ft, vd, wb, FZ, ks, Pp, wZ, cM, Ld, Zj, ar, W, aiS, it, ayV, fM, AL, jx, azF, j, qf, eA, J, o, XX, hb, Nc, kl, Y, r, en, iK, sn, auo, Dy, cS, dy, fD, bFR, gx, Gl, id, akd, bs, iG, ah, hF, dP, fU, io, aOh, lU, BN, eH, Sn, cr, qa, TA, Zi, bkh, Gu, hE, G, pf, qx, eb, fw, aR, aEQ, dY, il, hm, ayT, zQ, aW, Rr, OR, yd, eM, iT, Jp, uJ, pw, aWG, fr, qN, ai, FY, co, cc, h, dL, hB, Gc, aq, dw, Dy, vD, hk, uw, hR, uz, YH, bFT, bL, eR, yk, hy, ga, uT, aoA, afQ, aT, vH, bK, an, af, qw, s, ND, iU, hf, f, sS, nM, pZ, aJt, hS, qb, aD, ha, lD, jE, kL, av, F, NS, Od, fb, eZ, gl, Ej, aF, aot, bmH, eK, es, apM, bE, z, X); #c1(GSTT1) c2(NP_001280742) c3(glutathione S-transferase theta-1 isoform d) c4(111594) c5(111595, 111596, 111597, 111598, 111599, 111600) c6( ) c7(T, ea, fd, ci, aY, aE, hu, hH, az, Qv, lk, fT, iA, nB, cj, k, eg, cE, Uc, ht, aj, br, Gw, mJ, pl, ge, fe, pm, bbz, dB, bFW, bFV, abW, aAE, jv, aA, GP, cz, bX, Eh, q, akU, jj, sR, sX, eu, bA, avV, aMA, lo, cl, pb, ac, ig, mG, bt, rA, ao, gV, iW, jh, KO, oZ, uu, fu, asl, Gn, ad, hX, iC, yt, oW, lQ, NP, ZE, BO, cW, bFM, ff, gK, Ce, bpA, EZ, iV, Sm, bz, iq, oQ, i, Pk, iu, ne, rk, bM, if, e, bB, cL, bh, sD, Tz, ip, x, Na, bFU, ja, bc, dH, JI, fc, hl, kt, NR, as, eE, eF, bw, qJ, ii, vy, dD, gU, n, oY, awq, b, Bj, gd, aMj, eV, hn, aOX, A, fJ, AQ, bpx, cv, abV, hL, at, ut, B, pF, aCw, eX, fF, v, Qo, V, eJ, ae, jf, hM, HL, bu, t, ck, gX, Yz, vi, HQ, eq, Fc, hd, Db, Hk, hD, d, hG, gW, Z, bF, pP, y, pC, atT, aC, fR, atj, al, lj, qj, K, wt, dF, xa, eL, aAX, ml, wF, LZ, hN, aB, g, uC, ou, uo, dW, bFS, m, FW, sQ, sw, Ol, aw, fN, qy, BH, pM, ft, vd, wb, FZ, ks, Pp, wZ, cM, Ld, Zj, ar, W, aiS, it, ayV, fM, AL, jx, azF, j, qf, eA, J, o, XX, hb, Nc, kl, Y, r, en, iK, sn, auo, Dy, cS, dy, fD, bFR, gx, Gl, id, akd, bs, iG, ah, hF, dP, fU, io, aOh, lU, BN, eH, Sn, cr, qa, TA, Zi, bkh, Gu, hE, G, pf, qx, eb, fw, aR, aEQ, dY, il, hm, ayT, zQ, aW, Rr, OR, yd, eM, iT, Jp, uJ, pw, aWG, fr, qN, ai, FY, co, cc, h, dL, hB, Gc, aq, dw, Qy, vD, hk, uw, hR, uz, YH, bFT, bL, eR, yk, hy, ga, uT, aoA, afQ, aT, vH, bK, an, af, qw, s, ND, iU, hf, f, sS, nM, pZ, aJt, hS, qb, aD, ha, lD, jE, kL, av, F, NS, Od, fb, eZ, gl, Ej, aF, aot, bmH, ek, es, apM, bE, z, X); #c1(GSTT1) c2(NP_001280743) c3(glutathione S-transferase theta-1 isoform e) c4(111601) c5(111602, 111603, 111604, 111605, 111606) c6( ) c7(T, ea, fd, ci, aY, aE, hu, hH, az, Qv, lk, fT, iA, nB, cj, k, eg, cE, Uc, ht, aj, br, Gw, mJ, pl, ge, fe, pm, bbz, dB, bFW, bFV, abW, aAE, jv, aA, GP, cz, bX, Eh, q, akU, jj, sR, sX, eu, bA, avV, aMA, lo, cl, pb, ac, ig, mG, bt, rA, ao, gV, iW, jh, KO, oZ, uu, fu, asl, Gn, ad, hX, iC, yt, oW, lQ, NP, ZE, BO, cW, bFM, ff, gK, Ce, bpA, EZ, iV, Sm, bz, iq, oQ, i, Pk, iu, ne, rk, bM, if, e, bB, cL, bh, sD, Tz, ip, x, Na, bFU, ja, bc, dH, JI, fc, hl, kt, NR, as, eE, eF, bw, qJ, ii, vy, dD, gU, n, oY, awq, b, Bj, gd, aMj, eV, hn, aOX, A, fJ, AQ, bpx, cv, abV, hL, at, ut, B, pF, aCw, eX, fF, v, o, V, eJ, ae, jf, hM, HL, bu, t, ck, gX, Yz, vi, HO, eq, Fc, hd, Db, Hk, hD, d, hG, gW, Z, bF, pP, y, pC, atT, aC, fR, atj, al, lj, qj, K, wt, dF, xa, eL, aAX, mQ, wF, LZ, hN, aB, g, uC, ou, uo, dW, bFS, m, FW, sl, sw, OI, aw, fN, qy, BH, pM, ft, vd, wb, FZ, ks, Pp, wZ, cM, Ld, Zj, ar, W, aiS, it, ayV, fM, AL, jx, azF, j, qf, eA, J, o, XX, hb, Nc, kl, Y, r, en, iK, sn, auo, Dy, cS, dy, fD, bFR, gx, Gl, id, akd, bs, iG, ah, hF, dP, fU, io, aOh, lU, BN, eH, Sn, cr, qa, TA, Zi, bkh, Gu, hE, G, pf, qx, eb, fw, aR, aEQ, dY, il, hm, ayT, zQ, aW, Rr, OR, yd, eM, iT, Jp, uJ, pw, aWG, fr, qN, ai, FY, co, cc, h, dL, hB, Gc, aq, dw, Qy, vD, hk, uw, hR, uz, YH, bFT, bL, eR, yk, hy, ga, uT, aoA, afQ, aT, vH, bK, an, af, qw, s, ND, iU, hf, f, sS, nM, pZ, aJt, hS, qb, aD, ha, lD, jE, kL, av, F, NS, Od, fb, eZ, gl, Ej, af, aot, bmH, ek, es, apM, bE, z, X); #c1(GSTT2) c2(NP_000845) c3(glutathione S-transferase theta-2 isoform a) c4(16543) c5(16544, 16545, 16546, 16547, 16548, 16549) c6( ) c7(GI, ny, fT, ib, fR, ht, Gs, r, cz, rx, hy, Gw, K, q, bU, Gu, cM, xa); #c1(GSTT2B) c2(NP_001074312) c3(glutathione S-transferase theta-2B) c4(37082) c5(37083, 37084, 37085, 37086, 37087, 37088) c6( ) c7(T, ff, r, K, en, q); #c1(GSTZ1) c2(NP_665877) c3(maleylacetoacetate isomerase isoform 1) c4(241904, 241905, 241906) c5(241907, 241908, 241909, 241910, 241911, 241912, 241913, 241914, 241915, 241916, 241917) c6( ) c7(qw, SE, aHV, aW, aRa, cj, aT, at, uJ, dL, bK, ks, bX, an, Th, aax, eL); #c1(GSTZ1) c2(NP_665878) c3(maleylacetoacetate isomerase isoform 2) c4(241918) c5(241919, 241920, 241921, 241922, 241923, 241924) c6( ) c7(qw, SE, aHV, aW, aRa, cj, aT, at, uJ, dL, bK, ks, bX, an, Th, aax, eL); #c1(GSX1) c2(NP_663632) c3(GS homeobox 1) c4(241382) c5(241383, 241384, 241385, 241386, 241387, 241388) c6( ) c7(alv); #c1(GSX2) c2(NP_573574) c3(GS homeobox 2) c4(234609) c5(234610, 234611, 234612, 234613, 234614, 234615, 234616, 234617, 234618, 234619) c6( ) c7(PJ); #c1(GTDC1) c2(NP_001006637) c3(glycosyltransferase-like domain-containing protein 1 isoform a) c4(22257) c5(22258, 22259, 22260, 22261, 22262, 22263, 22264, 22265, 22266, 22267, 22268, 22269, 22270, 22271, 22272, 22273, 22274, 22275) c6( ) c7(ff, ht, en); #c1(GTDC1) c2(NP_001271164) c3(glycosyltransferase-like domain-containing protein 1 isoform e) c4(103503, 103504) c5(103505, 103506, 103507, 103508, 103509, 103510, 103511, 103512, 103513, 103514, 103515, 103516) c6( ) c7(ff, ht, en); #c1(GTDC1) c2(NP_078935) c3(glycosyltransferase-like domain-containing protein 1 isoform b) C4(216169) c5(216170, 216171, 216172, 216173, 216174, 216175, 216176, 216177, 216178, 216179) c6( ) c7(ff, ht, en); #c1(GTF2A1) c2(NP_056943) c3(transcription initiation factor IIA subunit 1 isoform 1) c4(188160, 188161, 188162) c5(188163, 188164, 188165, 188166, 188167, 188168, 188169, 188170) c6( ) c7(eD, aA, h, aT, aF, hp); #c1(GTF2A1) c2(NP_963889) c3(transcription initiation factor IIA subunit 1 isoform 2) c4( ) c5(262531, 262532, 262533, 262534, 262535, 262536, 262537, 262538, 262539, 262540, 262541) c6( ) c7(eD, aA, h, aT, aF, hp); #c1(GTF2A1L) c2(NP_00180416) c3(TFIIA-alpha and beta-like factor isoform 2) c4(74005) c5(74006, 74007, 74008, 74009, 74010, 74011) c6( ) c7(Ki, gU, ND, jx, hl, nA, B, vi); #c1(GTF2A1L) c2(NP_006863) c3(TFIIA-alpha and beta-like factor isoform 1) c4(168702, (68703) c5(168704, 168705, 168706, 168707, 168708, 168709, 168710, 168711, 168712, 168713, 168714, 168715) c6( ) c7(Ki, gU, ND, jx, hl, nA, B, vi); #c1(GTF2A2) c2(NP_004483) c3(transcription initiation factor IIA subunit 2) c4(146794) c5(146795, 146796, 146797, 146798, 146799, 146800, 146801, 146802, 146803, 146804, 146805, 146806, 146807, 146808) c6( ) c7(h, eD); #c1(GTF2E1) c2(NP_005504) c3(general transcription factor IIE subunit 1) c4(156873, 156874, 156875) c5(156876, 156877, 156878, 156879, 156880, 156881, 156882, 156883, 156884, 156885, 156886, 156887, 156888, 156889, 156890, 156891, 156892, 156893, 156894) c6( ) c7(aT); #c1(GTF2E2) c2(NP_002086) c3(transcription initiation factor IIE subunit beta) c4(124372, 124373, 124374) c5(124375, 124376, 124377, 124378, 124379, 124380, 124381) c6( ) c7(iV); #c1(GTF2E2) c2(XP_011542812) c3(transcription initiation factor IIE subunit beta isoform X2) c4( ) c5(341438, 341439, 341440, 341441, 341442, 341443, 341444, 341445, 341446, 341447, 341448, 341449) c6( ) c7(iV); #c1(GTF2F1) c2(NP_002087) c3(general transcription factor IIF subunit 1) c4(124382, 124383, 124384) c5(124385, 124386, 124387, 124388, 124389, 124390, 124391, 124392, 124393, 124394, 124395, 124396, 124397, 124398, 124399, 124400, 124401, 124402, 124403) c6( ) c7(m, aT); #c1(GTF2F2) c2(NP_004119) c3(general transcription factor IIF subunit 2) c4(143712) c5(143713, 143714, 143715, 143716, 143717, 143718, 143719) c6( ) c7(m); #c1(GTF2H1) c2(NP_001135779) c3(general transcription factor IIH subunit 1) c4(53220, 53221, 53222) c5(53223, 53224, 53225, 53226, 53227, 53228, 53229, 53230, 53231, 53232, 53233, 53234, 53235, 53236, 53237, 53238, 53239, 53240, 53241, 53242, 53243, 53244, 53245) c6( ) c7(bM, T, if, GC, To, v, BF, ci, bX, V, q, ad, eW, Kx, ke, av, jz, no, cF, zw, WY, aT, h, K, lQ, Wr, ae, af, d, aW, aBq, ff, az, alR, CT, r, aq, aaE, kp, La, en, g, bz, dr, bb, kP, c, eM, U, Kj, bdR, bi, xg, B); #c1(GTF2H2) c2(NP_001506) c3(general transcription factor IIH subunit 2) c4(118253, 118254) c5(118255, 118256, 118257, 118258, 118259, 118260, 118261, 118262, 118263, 118264, 118265, 118266, 118267, 118268, 118269, 118270, 118271, 118272) c6( ) c7(ke, G, T, no, dA, ad, apl, arX, aBq, wu, CT, kf, lQ, bdR, bu, La, wm, ae, dr, VF); #c1(GTF2H2C) c2(XP_005248650) c3(general transcription factor IIH subunit 2-like protein isoform X6) c4( ) c5(268487, 268488, 268489, 268490, 268491, 268492) c6( ) c7(ke, G, T, no, dA, ad, wu, aBq, CT, kf, lQ, La, bu, wm, ae, dr, VF); #c1(GTF2H2C) c2(XP_005276846) c3(general transcription factor IIH subunit 2-like protein isoform X6) c4( ) c5(285736, 285737, 285738, 285739, 285740, 285741) c6( ) c7(ke, G, T, no, dA, ad, wu, aBq, CT, kf, lQ, La, bu, wm, ae, dr, VF); #c1(GTF2H2C_2) c2(NP_001035955) c3(general transcription factor IIH subunit 2-like protein) c4(33522, 33523) c5(33524, 33525, 33526, 33527, 33528, 33529, 33530, 33531, 33532, 33533, 33534, 33535, 33536, 33537) c6( ) c7(ke, G, T, no, dA, ad, wu, aBq, CT, kf, lQ, La, bu, wm, ae, dr, VF); #c1(GTF2H3) c2(NP_001258796) c3(general transcription factor IIH subunit 3 isoform c) c4( ) c5(96546, 96547, 96548, 96549, 96550, 96551, 96552, 96553, 96554, 96555) c6( ) c7(bF, ke, T, ae, no, bE, aBq, CT, bB, lQ, bC, La, bD, pm, bz, dr, ad); #c1(GTF2H3) c2(NP_001507) c3(general transcription factor IIH subunit 3 isoform a) c4(118273, 118274, 118275) c5(118276, 118277, 118278, 118279, 118280, 118281, 118282, 118283) c6( ) c7(bF, ke, T, ae, no, bE, aBq, CT, bB, lQ, bC, La, bD, pm, bz, dr, ad); #c1(GTF2H4) c2(NP_001508) c3(general transcription factor IIH subunit 4) c4(118284, 118285, 118286) c5(118287, 118288, 118289, 118290, 118291, 118292, 118293, 118294, 118295, 118296, 118297, 118298, 118299, 118300, 118301, 118302, 118303, 118304, 118305, 118306, 118307, 118308, 118309, 118310, 118311, 118312, 118313, 118314, 118315, 118316, 118317, 118318, 118319, 118320, 118321, 118322, 118323, 118324, 118325, 118326, 118327, 118328, 118329, 118330, 118331, 118332, 118333, 118334, 118335, 118336, 118337, 118338, 118339, 118340, 118341, 118342, 118343, 118344, 118345, 118346, 118347, 118348, 118349, 118350, 118351, 118352, 118353, 118354, 118355, 118356, 118357, 118358, 118359, 118360, 118361, 118362, 118363, 118364, 118365, 118366, 118367, 118368, 118369, 118370, 118371, 118372, 118373, 118374, 118375, 118376, 118377) c6( ) c7(ke, T, aC, no, aE, bE, aBq, CT, dV, eZ, h, ci, lQ, La, g, V, ae, dr, ad); #c1(GTF2H5) c2(NP_997001) c3(general transcription factor IIH subunit 5) c4(264932) c5(264933, 264934, 264935, 264936, 264937, 264938) c6( ) c7(ke, T, ae, no, aBq, yk, rC, nl, nh, r, afB, uz, CT, lQ, La, bnB, V, q, dr, ad, bnA); #c1(GTF21) c2(NP_001267729) c3(general transcription factor II-1 isoform 6) c4( ) c5(99666, 99667, 99668, 99669, 99670, 99671, 99672, 99673, 99674, 99675, 99676, 99677) c6( ) c7(agK, ae, T, alC, VX, Az, fN, bhG, K, nA, ci, anN, bt, xg, ad, hq); #c1(GTF21) c2(NP_001509) c3(general transcription factor II-1 isoform 4) c4(118378, 118379) c5(118380, 118381, 118382, 118383, 118384, 118385) c6( ) c7(agK, ae, T, alC, VX, Az, fN, bhG, K, nA, ci, anN, bt, xg, ad, hq); #c1(GTF21) c2(NP_127492) c3(general transcription factor II-1 isoform 1) c4(226509, 226510) c5(226511, 226512, 226513, 226514, 226515, 226516, 226517, 226518, 226519, 226520, 226521, 226522) c6( ) c7(agK, ae, T, alC, VX, Az, fN, bhG, K, nA, ci, anN, bt, xg, ad, hq); #c1(GTF21) c2(NP_127493) c3(general transcription factor II-1 isoform 2) c4(226523, 226524) c5(226525, 226526, 226527, 226528, 226529, 226530) c6( ) c7(agK, ae, T, alC, VX, Az, fN, bhG, K, nA, ci, anN, bt, xg, ad, hq); #c1(GTF21) c2(NP_127494) c3(general transcription factor II-1 isoform 3) c4(226531) c5(226532, 226533, 226534, 226535, 226536, 226537) c6( ) c7(agK, ae, T, alC, VX, Az, fN, bhG, K, nA, ci, anN, bt, xg, ad, hq); #c1(GTF21RD1) c2(NP_001186136) c3(general transcription factor II-1 repeat domain-containing protein 1 isoform 3) c4(77190) c5(77191, 77192, 77193, 77194, 77195, 77196) c6( ) c7(av, alC, azj, Qu, ff, Az, Fl, en, bz, bwa, xg); #c1(GTF21RD1) c2(NP_005676) c3(general transcription factor II-1 repeat domain-containing protein 1 isoform 2) c4(158436) c5(158437, 158438, 158439, 158440, 158441, 158442, 158443) c6( ) c7(av, alC, azj, Qu, ff, Az, Fl, en, bz, bwa, xg); #c1(GTF21RD1) c2(NP_057412) c3(general transcription factor II-1 repeat domain-containing protein 1 isoform 1) c4(190627) c5(190628, 190629, 190630, 190631, 190632, 190633) c6( ) c7(av, alC, azj, Qu, ff, Az, Fl, en, bz, bwa, xg); #c1(GTF21RD2) c2(NP_775808) c3(general transcription factor II-1 repeat domain-containing protein 2A isoform 1) c4(250362) c5(250363, 250364, 250365, 250366, 250367, 250368, 250369, 250370, 250371, 250372, 250373, 250374, 250375, 250376, 250377, 250378, 250379, 250380, 250381) c6( ) c7(alC); #c1(GTF21RD2) c2(XP_011514922) c3(general transcription factor II-1 repeat domain-containing protein ZA isoform X1) c4(307680) c5(307681, 307682) c6( ) c7(alC); #c1(GTF21RD2) c2(XP_011514923) c3(general transcription factor II-I repeat domain-containing protein 2A isoform X2) c4(307683) c5(307684, 307685) c6( ) c7(alC); #c1(GTF21RD2) c2(XP_011514927) c3(general transcription factor II-1 repeat domain-containing protein ZA isoform X6) c4( ) c5(307686) c6( ) c7(alC); #c1(GTF3A) c2(NP_002088) c3(transcription factor IIIA) c4(124404, 124405) c5(124406, 124407, 124408, 124409, 124410, 124411, 124412, 124413, 124414, 124415, 124416, 124417, 124418) c6( ) c7(T, dD, aW, ea, al, h, lo, v, eq, ae, ad); #c1(GTF3C1) c2(NP_001511) c3(general transcription factor 3C polypeptide 1 isoform 1) c4(118407, 118408) c5(118409, 118410, 118411, 118412, 118413, 118414, 118415, 118416, 118417, 118418, 118419, (18420, 118421) c6( ) c7(ady); #c1(GTF3C1) c2(XP_01544117) c3(general transcription factor 3C polypeptide 1 isoform X3) c4( ) c5(343277) c6( ) c7(ady); #c1(GTPBP1) c2(NP_004277) c3(GTP-binding protein 1) c4(144771) c5(144772, 144773, 144774, 144775, 144776, 144777) c6( ) c7(ag, ame); #c1(GTPBP3) c2(NP_001122327) c3(tRNA modification GTPase GTPBP3, mitochondrial isoform III) c4(46539) c5(46540, 46541, 46542, 46543, 46544, 46545, 46546, 46547, 46548, 46549, 46550, 46551) c6( ) c7(bL); #c1(GTPBP3) c2(NP_001182351) c3(tRNA modification GTPase GTPBP3, mitochondrial isoform VII) c4(74817) c5(74818, 74819, 74820, 74821, 74822, 74823) c6( ) c7(bL); #c1(GTPBP3) c2(NP_116009) c3(tRNA modification GTPase GTPBP3, mitochondrial isoform V) c4(225190, 225191) c5(225192, 225193, 225194, 225195, 225196, 225197) c6( ) c7(bL); #c1(GTPBP3) c2(NP_598399) c3(tRNA modification GTPase GTPBP3, mitochondrial isoform 1V) c4(235496) c5(235497, 235498, 235499, 235500, 235501, 235502) c6( ) c7(bL); #c1(GTPBP4) c2(NP_036473) c3(nucleolar GTP-binding protein 1) c4(174103, 174104, 174105, 174106) c5(174107, 174108, 174109, 174110, 174111, 174112, 174113, 174114, 174115, 174116, 174117, 174118, 174119, 174120, 174121, 174122, 174123, 174124, 174125) c6( ) c7(ka, T, eR, adi, aj, J, kH, aT, EL, af, bmc, d, aW, hf, cm, bz, EJ, nB, dB, ki, hR, HB, AN); #c1(GTSF1) c2(NP_653195) c3(gametocyte-specific factor 1) c4(239007) c5(239008, 239009, 239010, 239011, 239012, 239013, 239014, 239015, 239016, 239017) c6( ) c7(xR); #c1(GUCA1A) c2(NP_000400) c3(guanylyl cyclase-activating protein 1) c4(8509, 8510, 8511) c5(8512, 8513, 8514, 8515, 8516, 8517, 8518, 8519, 8520, 8521, 8522, 8523, 8524, 8525, 8526, 8527, 8528, 8529, 8530, 8531, 8532, 8533, 8534, 8535, 8536, 8537, 8538, 8539, 8540, 8541, 8542, 8543) c6( ) c7(nJ, BI, IR, W, wu, bFX, Sr, nx, nw, bfR, wm, OL, Nh, on, nC); #c1(GUCA1B) c2(NP_002089) c3(guanylyl cyclase-activating protein 2) c4(124419, 124420) c5(124421, 124422, 124423, 124424, 124425, 124426, 124427, 124428, 124429, 124430, 124431, 124432) c6( ) c7(bFY, nC, nw, lR, nx); #c1(GUCD1) c2(NP_001271183) c3(protein GUCD1 isoform e) c4(103517) c5( ) c6( ) c7(KM, B, c); #c1(GUCD1) c2(NP_001271186) c3(protein GUCD1 isoform h) c4( ) c5(103518, 103519, 103520, 103521, 103522, 103523) c6( ) c7(KM, B, c); #c1(GUCD1) c2(NP_113632) c3(protein GUCD1 isoform d) c4(221831) c5(221832, 221833, 221834, 221835, 221836, 221837) c6( ) c7(KM, B, c); #c1(GUCY1A2) c2(NP_000846) c3(guanylate cyclase soluble subunit alpha-2 isoform 2) c4(16550) c5(16551, 16552, 16553, 16554, 16555, 16556) c6( ) c7(aw, aT); #c1(GUCY1A2) c2(NP_001243353) c3(guanylate cyclase soluble subunit alpha-2 isoform 1) c4(90482) c5(90483, 90484, 90485, 90486, 90487, 90488) c6( ) c7(aw, al); #c1(GUCY1A3) c2(NP_001124157) c3(guanylate cyclase soluble subunit alpha-3 isoform B) c4( ) c5(48313, 48314, 48315, 48316, 48317) c6( ) c7(bGa, oe, bFZ, qK, aw, cz, j, mq, bd, eA, X); #c1(GUCY1A3) c2(NP_001124159) c3(guanylate cyclase soluble subunit alpha-3 isoform D) c4(48318) c5(48319, 48320, 48321, 48322, 48323, 48324) c6( ) c7(bGa, oe, bFZ, qK, aw, cz, j, mq, bd, eA, X); #c1(GUCY1A3) c2(NP_001243378) c3(guanylate cyclase soluble subunit alpha-3 isoform A) c4(90632, 90633) c5(90634, 90635, 90636, 90637, 90638, 90639, 90640, 90641, 90642, 90643, 90644, 90645, 90646, 90647, 90648, 90649, 90650, 90651, 90652, 90653, 90654, 90655, 90656) c6( ) c7(bGa, oe, bFZ, qK, aw, cz, j, mq, bd, eA, X); #c1(GUCY1B3) c2(NP_000848) c3(guanylate cyclase soluble subunit beta-l isoform 2) c4(16557) c5(16558, 16559, 16560, 16561, 16562, 16563, 16564) c6( ) c7(t, m); #c1(GUCY1B3) c2(NP_001278881) c3(guanylate cyclase soluble subunit beta-1 isoform 3) c4(110776, 110777) c5(110778, 110779, 110780, 110781, 110782, 110783, 110784, 110785, 110786, 110787, 110788, 110789, 110790, 110791, 110792) c6( ) c7(t, m); #c1(GUCY2C) c2(NP_004954) c3(heat-stable enterotoxin receptor precursor) c4(151438) c5(151439, 151440, 151441, 151442, 151443, 151444) c6( ) c7(gh, T, aZ, baV, bO, cz, bB, n, bS, v, ct, q, qC, dP, bGb, gd, h, jH, bK, K, ji, hk, Qe, ak, avH, r, fe, g, Z, bGc, bF, aqp, bE, Ji, Rm, cE, ao); #c1(GUCY2D) c2(NP_000171) c3(retinal guanylyl cyclase 1) c4(3789) c5(3790, 3791, 3792, 3793, 3794, 3795) c6( ) c7(bGg, nx, bFp, jh, uu, ci, alw, Nh, bGe, bfR, bGd, OK, bFr, lR, nO, bGf, nC, ac, aVv, nw, eQ, OL, de); #c1(GUCY2F) c2(NP_001513) c3(retinal guanylyl cyclase 2) c4(118422) c5(118423, 118424, 118425, 118426, 118427, 118428) c6( ) c7(aC, Nh); #c1(GUK1) c2(NP_001152862) c3(guanylate kinase isoform a) c4(60200, 60201, 60202) c5(60203, 60204, 60205, 60206, 60207, 60208, 60209, 60210, 60211, 60212, 60213, 60214, 60215, 60216, 60217, 60218, 60219, 60220, 60221, 60222, 60223, 60224, 60225, 60226, 60227) c6( ) c7(eW, j, s, bq); #c1(GUK1) c2(NP_001152863) c3(guanylate kinase isoform b) c4(60228, 60229, 60230, 60231) c5(60232, 60233, 60234, 60235, 60236, 60237, 60238, 60239, 60240, 60241, 60242, 60243, 60244, 60245, 60246, 60247, 60248, 60249, 60250, 60251, 60252, 60253, 60254, 60255, 60256, 60257, 60258, 60259, 60260, 60261, 60262, 60263) c6( ) c7(eW, j, s, bq); #c1(GUK1) c2(NP_001229769) c3(guanylate kinase isoform c) c4(85222) c5(85223, 85224, 85225, 85226, 85227, 85228) c6( ) c7(eW, j, s, bq); #c1(GUK1) c2(XP_005273160) c3(guanylate kinase isoform X1) c4(284312) c5(284313, 284314, 284315, 284316, 284317, 284318) c6( ) c7(eW, j, s, bq); #c1(GUK1) c2(XP_005273161) c3(guanylate kinase isoform X2) c4(284319) c5(284320, 284321, 284322, 284323, 284324, 284325) c6( ) c7(eW, j, s, bq); #c1(GUK1) c2(XP_011542476) c3(guanylate kinase isoform X3) c4( ) c5(340973) c6( ) c7(eW, j, s, bq); #c1(GULP1) c2(NP_001239597) c3(PTB domain-containing engulfment adapter protein 1 isoform b) c4( ) c5(88559, 88560, 88561, 88562, 88563, 88564, 88565, 88566, 88567, 88568) c6( ) c7(hz, oe, ir, g, cG); #c1(GULP1) c2(XP_006712653) c3(PTB domain-containing engulfment adapter protein 1 isoform X1) c4(286769, 286770, 286771) c5(286772, 286773, 286774, 286775, 286776, 286777, 286778, 286779, 286780, 286781, 286782, 286783, 286784, 286785, 286786, 286787, 286788, 286789) c6( ) c7(hz, oe, ir, g, cG); #c1(GULP1) c2(XP_011509637) c3(PTB domain-containing engulfment adapter protein 1 isoform X10) c4( ) c5(300211, 300212, 300213, 300214, 300215, 300216) c6( ) c7(hz, oe, ir, g, cG); #c1(GUSB) c2(NP_000172) c3(beta-glucuronidase isoform 1 precursor) c4(3796) c5(3797, 3798, 3799, 3800, 3801, 3802) c6( ) c7(T, aiR, q, aR, er, hX, iu, l, u, aT, h, dz, wt, iU, Y, r, aq, bGh, dr, bGi, ck, ajH, kg, ig, cj, Lp, Lq, anx, y, bt, B, vi); #c1(GUSB) c2(XP_005250354) c3(beta-glucuronidase isoform X1) c4(270260) c5(270261, 270262, 270263, 270264, 270265, 270266) c6( ) c7(T, aiR, q, aR, er, hX, iu, l, u, aT, h, dz, wt, iU, Y, r, aq, bGh, dr, bGi, ck, ajH, kg, ig, cj, Lp, Lq, anx, y, bt, B, vi); #c1(GYG1) c2(NP_001171649) c3(glycogenin-1 isoform 2) c4(71140) c5(71141, 71142, 71143, 71144, 71145, 71146) c6( ) c7(Bw, j, bGj); #c1(GYG1) c2(NP_001171650) c3(glycogenin-1 isoform 3) c4(71147) c5(71148, 71149, 71150, 71151, 71152, 71153) c6( ) c7(Bw, j, bGj); #c1(GYG1) c2(NP_004121) c3(glycogenin-I isoform 1) c4(143720) c5(143721, 143722, 143723, 143724, 143725, 143726) c6( ) c7(Bw, j, bGj); #c1(GYG2) c2(NP_001073324) c3(glycogenin-2 isoform a) c4( ) c5(35846, 35847, 35848, 35849, 35850, 35851) c6( ) c7(ajU); #c1(GYG2) c2(NP_001171631) c3(glycogenin-2 isoform c) c4(71057, 71058, 71059, 71060) c5(71061, 71062, 71063, 71064, 71065, 71066, 71067, 71068, 71069, 71070, 71071, 71072, 71073, 71074, 71075, 71076, 71077, 71078) c6( ) c7(ajU); #c1(GYG2) c2(NP_001171632) c3(glycogenin-2 isoform d) c4(71079) c5(71080, 71081, 71082, 71083, 71084, 71085) c6( ) c7(ajU); #c1(GYG2) c2(NP_003909) c3(glycogenin-2 isoform b) c4(142092, 142093) c5(142094, 142095, 142096, 142097, 142098, 142099) c6( ) c7(ajU); #c1(GYG2) c2(XP_006724584) c3(glycogenin-2 isoform X2) c4(296095) c5(296096, 296097, 296098, 296099, 296100, 296101) c6( ) c7(ajU); #c1(GYG2) c2(XP_011543903) c3(glycogenin-2 isoform X3) c4(343092) c5(343093, 343094, 343095, 343096, 343097, 343098, 343099, 343100, 343101, 343102, 343103, 343104) c6( ) c7(ajU); #c1(GYLTL1B) c2(NP_001287650) c3(glycosyltransferase-like protein LARGE2 isoform a precursor) c4(112420) c5(112421, 112422, 112423, 112424, 112425, 112426, 112427, 112428, 112429, 112430, 112431, 112432, 112433, 112434, 112435, 112436, 112437, 112438, 112439, 112440) c6( ) c7(T, ac, xs, aT, dy, ci, ae, ad); #c1(GYLTL1B) c2(NP_001287651) c3(glycosyltransferase-like protein LARGE2 isoform b) c4(112441) c5( ) c6( ) c7(T, ac, xs, aT, dy, ci, ae, ad); #c1(GYLTL1B) c2(XP_006718204) c3(glycosyltransferase-like protein LARGE2 isoform X8) c4(291576) c5(291577, 291578, 291579, 291580, 291581, 291582) c6( ) c7(T, ac, xs, aT, dy, ci, ae, ad); #c1(GYLTL1B) c2(XP_011518192) c3(glycosyltransferase-like protein LARGE2 isoform X5) c4( ) c5(312237) c6( ) c7(T, ac, xs, aT, dy, ci, ae, ad); #c1(GYLTL1B) c2(XP_011518195) c3(glycosyltransferase-like protein LARGE2 isoform X9) c4( ) c5(312238) c6( ) c7(T, ac, xs, aT, dy, ci, ae, ad); #c1(GYPA) c2(NP_002090) c3(glycophorin-A precursor) c4(124433, 124434, 124435) c5(124436, 124437, 124438, 124439, 124440, 124441, 124442, 124443, 124444, 124445, 124446, 124447, 124448, 124449, 124450, 124451, 124452, 124453) c6( ) c7(bM, T, os, qo, dM, lo, SO, qd, aw, cz, aj, bS, oZ, ct, x, oT, no, asY, dV, hD, bK, adV, MY, wr, amx, Fm, lW, aEW, La, pm, aot, Z, ve, bw, bz, aYt, ck, TA, VJ, hk, at, dW, bC, lf, bt, fh); #c1(GYPB) c2(NP_002091) c3(glycophorin-B isoform 1 precursor) c4(124454, 124455) c5(124456, 124457, 124458, 124459, 124460, 124461, 124462, 124463, 124464, 124465, 124466, 124467, 124468, 124469, 124470, 124471, 124472, 124473, 124474, 124475, 124476, 124477, 124478, 124479, 124480, 124481, 124482, 124483) c6( ) c7(TA, qo, VJ, amx, asY, Fm, lW, dV, cz, hD, fN, aEW, bS, ct, aot, Z); #c1(GYPB) c2(XP_011530205) c3(glycophorin-B isoform X1) c4(326124, 326125) c5( ) c6( ) c7(TA, qo, VJ, amx, asY, Fm, lW, dV, cz, hD, fN, aEW, bS, ct, aot, Z); #c1(GYPC) c2(NP_001243513) c3(glycophorin-C isoform 3) c4(90981) c5(90982, 90983, 90984, 90985, 90986) c6( ) c7(bpn, xK, bsd, dV, mL, m, k, qs, cd); #c1(GYPC) c2(NP_002092) c3(glycophorin-C isoform 1) c4(124484) c5(124485, 124486, 124487, 124488, 124489, 124490, 124491) c6( ) c7(bpn, xK, bsd, dV, mL, m, k, qs, cd); #c1(GYPC) c2(NP_058131) c3(glycophorin-C isoform 2) c4(192646) c5(192647, 192648, 192649, 192650, 192651, 192652) c6( ) c7(bpn, xK, bsd, dV, mL, m, k, qs, cd); #c1(GYPE) c2(NP_002093) c3(glycophorin-E precursor) c4(124492, 124493) c5(124494, 124495, 124496, 124497, 124498, 124499, 124500, 124501, 124502, 124503, 124504, 124505, 124506, 124507, 124508, 124509) c6( ) c7(TA, Qi, qo, amx, asY, ex, lW, dV, cz, hD, aEW, bS, ct, aot, Z); #c1(GYS1) c2(NP_001155059) c3(glycogen [starch] synthase, muscle isoform 2) c4(61426, 61427) c5(61428, 61429, 61430, 61431, 61432, 61433, 61434, 61435, 61436, 61437, 61438, 61439) c6( ) c7(bL, bGk, cc, eR, aw, hl, cz, dG, ei, nS); #c1(GYS1) c2(NP_002094) c3(glycogen [starch] synthase, muscle isoform 1) c4(124510, 124511, 124512) c5(124513, 124514, 124515, 124516, 124517, 124518, 124519, 124520, 124521, 124522, 124523, 124524, 124525, 124526, 124527, 124528, 124529, 124530) c6( ) c7(bL, bGk, cc, eR, aw, hl, cz, dG, ei, nS); #c1(GYS2) c2(NP_068776) c3(glycogen [starch] synthase, liver) c4(210363, 210364) c5(210365, 210366, 210367, 210368, 210369, 210370, 210371, 210372, 210373, 210374, 210375, 210376, 210377, 210378, 210379, 210380, 210381, 210382, 210383, 210384, 210385, 210386, 210387, 210388, 210389, 210390, 210391, 210392, 210393, 210394, 210395, 210396, 210397, 210398, 210399, 210400, 210401, 210402, 210403, 210404, 210405, 210406, 210407, 210408, 210409, 210410, 210411, 210412, 210413, 210414, 210415, 210416, 210417, 210418, 210419, 210420, 210421, 210422, 210423, 210424, 210425, 210426, 210427, 210428, 210429, 210430, 210431, 210432, 210433, 210434, 210435, 210436, 210437, 210438, 210439, 210440, 210441, 210442, 210443, 210444, 210445, 210446, 210447, 210448, 210449, 210450, 210451, 210452, 210453, 210454, 210455, 210456, 210457) c6(210458) c7(bL, bGl, kl, blo, aZ, qQ, hl, mg, v); #c1(GZF1) c2(XP_005260860) c3(GDNF-inducible zinc finger protein 1 isoform X5) c4(276937) c5(276938, 276939, 276940, 276941, 276942, 276943) c6( ) c7(fq); #c1(GZMA) c2(NP_006135) c3(granzyme A precursor) c4(162296) c5(162297, 162298, 162299, 162300, 162301, 162302) c6( ) c7(x, bM, Dw, EM, fR, ig, aHW, ds, fN, at, h, Ds, lR, k, y, pb, boN); #c1(GZMB) c2(NP_004122) c3(granzyme B precursor) c4(143727, 143728, 143729, 143730) c5(143731, 143732, 143733, 143734, 143735, 143736, 143737, 143738, 143739, 143740, 143741, 143742, 143743, 143744, 143745, 143746, 143747, 143748) c6( ) c7(oO, bM, T, lD, xS, fR, Zz, ea, qx, hE, cL, qj, ci, x, aE, fS, Ds, bGm, eL, adT, cp, ly, aW, mQ, aB, bw, cj, k, m, dD, n, fr, qN, ju, cU, h, eV, dL, bxg, A, fJ, lb, fe, lx, bm, at, Dw, eX, fF, C, q, o, aT, rz, ae, af, hM, bf, iU, axW, Y, r, lo, en, aER, bN, ck, OW, dh, dy, fD, rA, bt, Ke, jh, wV, ah, jE, Gn, EA, eq, pz, ad, hX, F, ds, aak, uD, ff, cr, eZ, iV, aS, lf, y); #c1(GZMB) c2(XP_011534987) c3(granzyme B isoform X1) c4(331541, 331542, 331543) c5(331544, 331545, 331546, 331547, 331548, 331549, 331550, 331551, 331552, 331553, 331554, 331555) c6( ) c7(oO, bM, T, lD, xS, fR, Zz, ea, qx, hE, cL, qj, ci, x, aE, fS, Ds, bGm, eL, adT, cp, ly, aW, mQ, aB, bw, cj, k, m, dD, n, fr, qN, ju, cU, h, eV, dL, bxg, A, fJ, lb, fe, lx, bm, at, Dw, ex, fF, C, q, o, aT, rz, ae, af, hM, bf, iU, axW, Y, r, lo, en, aER, bN, ck, OW, dh, dy, fD, rA, bt, Ke, jh, wV, ah, jE, Gn, EA, eq, pz, ad, hX, F, ds, aak, uD, ff, cr, eZ, iV, aS, lf, y); #c1(GZMH) c2(NP_001257710) c3(granzyme H isoform 3 precursor) c4(95604, 95605) c5(95606, 95607, 95608, 95609, 95610, 95611, 95612, 95613, 95614, 95615, 95616, 95617) c6( ) c7(iU, jx, bX); #c1(GZMH) c2(NP_219491) c3(granzyme H isoform 1 precursor) c4(228595) c5(228596, 228597, 228598, 228599, 228600, 228601) c6( ) c7(il, jx, bX); #c1(GZMK) c2(NP_002095) c3(granzyme K precursor) c4(124531) c5(124532, 124533, 124534, 124535, 124536, 124537, 124538, 124539) c6( ) c7(V, C); #c1(GZMM) c2(NP_005308) c3(granzyme M isoform 1 preproprotein) c4(154863) c5(154864, 154865, 154866, 154867, 154868, 154869) c6( ) c7(fM, iM, Ew, awS, je, oV, bO, kH, aT, ci, ik, ae, ad); #c1(H1F0) c2(NP_005309) c3(histone H1.0) c4(154870, 154871) c5(154872, 154873, 154874, 154875, 154876, 154877, 154878, 154879, 154880, 154881, 154882, 154883) c6( ) c7(ck, T, bP, aW, Dq, al, h, aq, bC, iV, t, bt, B, hX); #c1(H1FX) c2(NP_006017) c3(histone H1x) c4(161204) c5(161205, 161206, 161207, 161208, 161209, 161210) c6( ) c7(pX); #c1(H2AFJ) c2(NP_808760) c3(histone H2A.J) c4(252892, 252893, 252894, 252895) c5(252896, 252897, 252898, 252899, 252900, 252901, 252902, 252903, 252904, 252905, 252906, 252907, 252908, 252909, 252910, 252911) c6( ) c7(aW, aT); #c1(H2AFX) c2(NP_002096) c3(histone H2AX) c4(124540) c5(124541, 124542, 124543, 124544, 124545, 124546, 124547, 124548, 124549, 124550) c6( ) c7(T, os, fM, AS, pf, WL, iW, ew, cz, Hn, n, oZ, v, fd, ci, jh, V, q, ad, YD, eA, ft, qg, bGn, Bc, WY, ajv, aT, kf, vH, dL, AN, K, fb, ae, Zm, qw, d, wr, ND, iU, aW, Y, afF, ff, eZ, r, ks, aq, vs, xH, en, cl, pL, t, bw, bz, ek, adq, ck, qJ, rR, kP, eM, KB, bP, hL, eo, m, bC, bt, cB, B); #c1(H2AFY) c2(NP_004884) c3(core histone macro-H2A.1 isoform 2) c4(150538) c5(150539, 150540, 150541, 150542, 150543, 150544) c6( ) c7(T, B, bE, aW, if, eZ, aT, bB, ey, fL, qN, bz, mC, bN); #c1(H2AFY) c2(NP_613075) c3(core histone macro-H2A.1 isoform 1) c4(236776) c5(236777, 236778, 236779, 236780, 236781, 236782) c6( ) c7(T, B, bE, aW, if, eZ, aT, bB, ey, fL, qN, bz, mC, bN); #c1(H2AFY) c2(NP_613258) c3(core histone macro-H2A.1 isoform 3) c4(236783) c5(236784, 236785, 236786, 236787, 236788, 236789, 236790, 236791, 236792, 236793) c6( ) c7(T, B, bE, aW, if, eZ, aT, bB, ey, fL, qN, bz, mC, bN); #c1(H2AFY) c2(XP_005272189) c3(core histone macro-H2A.1 isoform X3) c4(283751) c5(283752, 283753, 283754, 283755, 283756, 283757) c6( ) c7(T, B, bE, aW, if, eZ, aT, bB, ey, fL, qN, bz, mC, bN); #c1(H2AFY2) c2(NP_061119) c3(core histone macro-H2A.2) c4(199207, 199208) c5(199209, 199210, 199211, 199212, 199213, 199214, 199215, 199216, 199217, 199218, 199219, 199220, 199221, 199222, 199223, 199224) c6( ) c7(bz, ad); #c1(H2AFZ) c2(NP_002097) c3(histone H2A.Z) c4(124551) c5(124552, 124553, 124554, 124555, 124556, 124557) c6( ) c7(T, aW, LX, aT, ey, ae, ad); #c1(H2BFWT) c2(NP_001002916) c3(histone H2B type W-T) c4(19770) c5(19771, 19772, 19773, 19774, 19775, 19776) c6( ) c7(vH, gU); #c1(H3F3B) c2(NP_005315) c3(histone H3.3) c4(154919, 154920) c5(154921, 154922, 154923, 154924, 154925, 154926, 154927, 154928, 154929, 154930, 154931, 154932, 154933, 154934, 154935, 154936, 154937, 154938, 154939, 154940, 154941, 154942, 154943, 154944) c6( ) c7(T, Uf, d, zQ, dB, aAz, zt, BK, qy, asV, ci, as, zP, eA); #c1(HAAO) c2(NP_036337) c3(3-hydroxyanthranilate 3,4-dioxygenase) c4(173070) c5(173071, 173072, 173073, 173074, 173075, 173076) c6( ) c7(c, hL, cl, V, ae, ad); #c1(HABP2) c2(NP_001171131) c3(hyaluronan-binding protein 2 isoform 2) c4( ) c5(69906, 69907, 69908, 69909, 69910, 69911, 69912, 69913, 69914, 69915) c6( ) c7(dD, ea, e, al, jx, fF, vr, n, aj, j, eq, J, M, bGo, ff, en, g, gk, kT, ac, ag, qq); #c1(HABP2) c2(NP_004123) c3(hyaluronan-binding protein 2 isoform 1 preproprotein) c4(143749, 143750, 143751) c5(143752, 143753, 143754, 143755, 143756, 143757, 143758, 143759, 143760, 143761, 143762, 143763, 143764, 143765) c6( ) c7(dD, ea, e, al, jx, fF, vr, n, aj, j, eq, J, M, bGo, ff, en, g, gk, kT, ac, ag, qq); #c1(HABP4) c2(NP_055097) c3(intracellular hyaluronan-binding protein 4) c4(178908, 178909) c5(178910, 178911, 178912, 178913, 178914, 178915, 178916, 178917) c6(178918, 178919) c7(X); #c1(HABP4) c2(XP_005251869) c3(intracellular hyaluronan-binding protein 4 isoform X1) c4( ) c5(271245, 271246, 271247, 271248, 271249, 271250, 271251, 271252) c6(271253, 271254) c7(X); #c1(HACD1) c2(NP_055056) c3(very-long-chain (3R)-3-hydroxyacyl-CoA dehydratase 1) c4(178638, 178639) c5(178640, 178641, 178642, 178643, 178644, 178645) c6( ) c7(bM, T, Ke, jx, q, eW, F, afn, aT, h, kl, avR, aW, Ad, r, tv, aq, tu, avT, cT, rR, avS, wK, aru, B); #c1(HACD1) c2(XP_005252698) c3(very-long-chain (3R)-3-hydroxyacyl-CoA dehydratase 1 isoform X1) c4( ) c5(272013, 272014, 272015, 272016, 272017, 272018) c6( ) c7(bM, T, Ke, jx, q, eW, F, afn, aT, h, kl, avR, aW, Ad, r, tv, aq, tu, avT, cT, rR, avS, wK, aru, B); #c1(HACD2) c2(NP_940684) c3(very-long-chain (3R)-3-hydroxyacyl-CoA dehydratase 2) c4(259843) c5(259844, 259845, 259846, 259847, 259848, 259849) c6( ) c7(cF); #c1(HACD4) c2(NP_001010915) c3(very-long-chain (3R)-3-hydroxyacyl-CoA dehydratase 4) c4(24160) c5(24161, 24162, 24163, 24164, 24165, 24166, 24167, 24168, 24169, 24/70) c6( ) c7(j); #c1(HACE1) c2(NP_065822) c3(E3 ubiquitin-protein ligase HACE1) c4(205685) c5(205686, 205687, 205688, 205689, 205690, 205691) c6( ) c7(T, r, ci, K, cm, q, er); #c1(HACL1) c2(NP_036392) c3(2-hydroxyacyl-CoA lyase 1 isoform a) c4(173559) c5(173560, 173561, 173562, 173563, 173564, 173565) c6( ) c7(qs); #c1(HADH) c2(NP_001171634) c3(hydroxyacyl-coenzyme A dehydrogenase, mitochondrial isoform 1 precursor) c4(71086) c5(71087, 71088, 71089, 71090, 71091, 71092) c6( ) c7(bL, aw, bo, hl, bGq, mE, jy, oh, v, mL, bq, bqa, dF, bGp); #c1(HADH) c2(NP_005318) c3(hydroxyacyl-coenzyme A dehydrogenase, mitochondrial isoform 2 precursor) c4(154962) c5(154963, 154964, 154965, 154966, 154967, 154968) c6( ) c7(bL, aw, bo, hl, bGq, mE, jy, oh, v, ml, bq, bqa, dF, bGp); #c1(HADH) c2(XP_005263029) c3(hydroxyacyl-coenzyme A dehydrogenase, mitochondrial isoform X1) c4(278205) c5(278206, 278207, 278208, 278209, 278210, 278211, 278212, 278213, 278214, 278215) c6( ) c7(bL, aw, bo, hl, bGq, mE, jy, oh, v, mL, bq, bqa, dF, bGp); #c1(HADHA) c2(NP_000173) c3(trifunctional enzyme subunit alpha, mitochondrial precursor) c4(3803, 3804) c5(3805, 3806, 3807, 3808, 3809, 3810, 3811, 3812, 3813, 3814, 3815, 3816) c6( ) c7(bGt, jv, AL, bGr, fF, v, j, bX, ad, av, b, cc, ZU, jy, dl, fc, hl, dq, EZ, bz, bGs, bo, at, i); #c1(HADHB) c2(NP_001268441) c3(trifunctional enzyme subunit beta, mitochondrial isoform 2 precursor) c4(99937) c5(99938, 99939, 99940, 99941, 99942, 99943) c6( ) c7(fc, aT, gZ, ZU, qA, dF); #c1(HADHB) c2(XP_011531105) c3(trifunctional enzyme subunit beta, mitochondrial isoform X1) c4(327035, 327036, 327037, 327038) c5(327039, 327040, 327041, 327042, 327043, 327044, 327045, 327046, 327047, 327048, 327049, 327050, 327051, 327052, 327053, 327054, 327055, 327056, 327057, 327058, 327059, 327060, 327061, 327062) c6( ) c7(fc, aT, gZ, ZU, qA, dF); #c1(HADHB) c2(XP_011531106) c3(trifunctional enzyme subunit beta, mitochondrial isoform X2) c4(327063) c5(327064, 327065, 327066, 327067, 327068, 327069) c6( ) c7(fc, aT, gZ, ZU, qA, dF); #c1(HAGH) c2(NP_001035517) c3(hydroxyacylglutathione hydrolase, mitochondrial isoform 2) c4( ) c5(32837, 32838, 32839, 32840, 32841) c6( ) c7(y, iA, aW, aT, fP, fr, K, g, lu, aY, ad); #c1(HAGH) c2(NP_005317) c3(hydroxyacylglutathione hydrolase, mitochondrial isoform 1 precursor) c4(154952, 154953) c5(154954, 154955, 154956, 154957, 154958, 154959, 154960, 154961) c6( ) c7(y, iA, aW, aT, fP, fr, K, g, lu, aY, ad); #c1(HAGH) c2(XP_011520772) c3(hydroxyacylglutathione hydrolase, mitochondrial isoform X1) c4(315779) c5(315780, 315781, 315782, 315783, 315784) c6( ) c7(y, iA, aW, aT, fP, fr, K, g, lu, aY, ad); #c1(HAL) c2(NP_001245262) c3(histidine ammonia-lyase isoform 2) c4(92951) c5(92952, 92953, 92954, 92955, 92956) c6( ) c7(eP, v, ci, Qo, mC, x, afQ, bpo, je, lW, ff, eH, eZ, en, eF, ck, iO, sS, iM, hk, fL, k, amb, iu); #c1(HAL) c2(NP_001245263) c3(histidine ammonia-lyase isoform 3) c4(92957) c5(92958, 92959, 92960, 92961, 92962, 92963) c6( ) c7(eP, v, ci, Qo, mC, x, afQ, bpo, je, lW, ff, eH, eZ, en, eF, ck, iO, sS, iM, hk, fL, k, amb, iu); #c1(HAL) c2(NP_002099) c3(histidine ammonia-lyase isoform 1) c4(124558) c5(124559, 124560, 124561, 124562, 124563, 124564, 124565) c6( ) c7(eP, v, ci, Qo, mC, x, afQ, bpo, je, lW, ff, eH, eZ, en, eF, ck, iO, sS, iM, hk, fL, k, amb, iu); #c1(HAMP) c2(NP_066998) c3(hepcidin preproprotein) c4(208092, 208093, 208094, 208095, 208096, 208097, 208098, 208099, 208100, 208101, 208102, 208103, 208104, 208105, 208106, 208107, 208108, 208109, 208110) c5(208111, 208112, 208113, 208114, 208115, 208116, 208117, 208118, 208119, 208120, 208121, 208122, 208123, 208124, 208125, 208126, 208127, 208128, 208129, 208130, 208131, 208132, 208133, 208134, 208135, 208136, 208137, 208138, 208139, 208140, 208141, 208142, 208143, 208144, 208145, 208146, 208147, 208148, 208149, 208150, 208151, 208152, 208153, 208154, 208155, 208156, 208157, 208158, 208159, 208160, 208161, 208162, 208163, 208164, 208165, 208166, 208167, 208168, 208169, 208/70, 208171, 208172, 208173, 208174, 208175, 208176, 208177, 208178, 208179, 208180, 208181, 208182, 208183, 208184, 208185, 208186, 208187, 208188, 208189, 208190, 208191, 208192, 208193, 208194, 208195, 208196, 208197, 208198, 208199, 208200, 208201, 208202, 208203, 208204, 208205, 208206, 208207, 208208) c6(208209) c7(oO, bGu, T, jv, bL, lk, fR, dD, qb, aZ, ea, aD, al, jx, eR, fd, aHv, zN, n, QZ, aj, jE, ml, v, Rc, aY, eg, eq, br, J, bGx, hX, fU, oT, F, yw, oz, BR, jZ, oW, aT, dV, yu, h, ji, K, aln, qM, qD, dF, kl, PK, s, aW, atl, hl, eZ, yy, hN, lo, aq, fe, jh, Fq, as, bw, gk, BU, aNv, xO, kz, aUW, QV, fP, dy, bGv, k, Mn, abX, pw, ei, alq, B, y, bGw); #c1(HAND1) c2(NP_004812) c3(heart- and neural crest derivatives-expressed protein 1) c4(149994) c5(149995, 149996, 149997, 149998, 149999, 150000, 150001) c6( ) c7(Mg, jf, T, cc, gY, acA, acl, mx, ll, aA, gZ, aj, aF, j, bD, hH, cd); #c1(HAND1) c2(XP_005268588) c3(heart- and neural crest derivatives-expressed protein 1 isoform X1) c4( ) c5(281936) c6( ) c7(Mg, jf, T, cc, gY, acA, acl, mx, ll, aA, gZ, aj, aF, j, bD, hH, cd); #c1(HAND2) c2(NP_068808) c3(heart- and neural crest derivatives-expressed protein 2) c4(210666, 210667, 210668) c5(210669, 210670, 210671, 210672, 210673, 210674, 210675, 210676, 210677, 210678, 210679, 210680, 210681, 210682, 210683, 210684, 210685, 210686, 210687) c6( ) c7(Mg, EW, cc, bjA, are, hL, aoW, zG, lQ, art, cl, bD, agY, cd); #c1(HAD1) c2(NP_060015) c3(hydroxyacid oxidase 1) c4(193659) c5(193660, 193661, 193662, 193663, 193664, 193665) c6( ) c7(ac, MB, k, ad, bpN); #c1(HAO2) c2(XP_011539860) c3(hydroxyacid oxidase 2 isoform X2) c4(338147) c5(338148, 338149, 338150, 338151, 338152, 338153) c6( ) c7(fQ, dL, MB, ac); #c1(HAP1) c2(NP_001073339) c3(huntingtin-associated protein 1 isoform 3) c4(35952) c5(35953, 35954, 35955, 35956, 35957, 35958) c6( ) c7(Zo, T, dD, gU, Zz, ea, ex, cz, fF, ah, ci, j, eq, il, ad, qh, HG, rN, eV, qM, aak, ae, bc, af, bA, bGy, uY, bz, qa, rR, bb); #c1(HAP1) c2(NP_001073340) c3(huntingtin-associated protein 1 isoform 4) c4(35959) c5(35960, 35961, 35962, 35963, 35964, 35965) c6( ) c7(Zo, T, dD, gU, Zz, ea, eX, cz, fF, ah, ci, j, eq, il, ad, qh, HG, rN, eV, qM, aak, ae, bc, af, bA, bGy, uY, bz, qa, rR, bb); #c1(HAP1) c2(NP_817084) c3(huntingtin-associated protein 1 isoform 2) c4(253068, 253069) c5(253070, 253071, 253072, 253073, 253074, 253075, 253076, 253077, 253078, 253079, 253080, 253081) c6( ) c7(Zo, T, dD, gU, Zz, ea, ex, cz, fF, ah, ci, j, eq, il, ad, qh, HG, rN, eV, qM, aak, ae, bc, af, bA, bGy, uY, bz, qa, rR, bb); #c1(HAPLN1) c2(NP_001875) c3(hyaluronan and proteoglycan link protein 1 precursor) c4(121885, 121886, 121887, 121888, 121889, 121890) c5(121891, 121892, 121893, 121894, 121895, 121896, 121897, 121898, 121899, 121900, 121901, 121902, 121903, 121904, 121905, 121906, 121907, 121908, 121909, 121910, 121911, 121912, 121913, 121914, 121915, 121916, 121917, 121918, 121919, 121920, 121921, 121922, 121923, 121924, 121925, 121926) c6( ) c7(ib, mR, rh, bE, nO, aw, ln, Az, bB, Da, y, cM, X, atb, ad); #c1(HAPLN4) c2(NP_075378) c3(hyaluronan and proteoglycan link protein 4 precursor) c4(213798) c5(213799, 213800, 213801, 213802, 213803, 213804, 213805, 213806, 213807, 213808) c6(213809, 213810) c7(eA); #c1(HARS) c2(NP_001244969) c3(histidine-tRNA ligase, cytoplasmic isoform 2) c4(92511, 92512) c5(92513, 92514, 92515, 92516, 92517, 92518, 92519, 92520, 92521, 92522, 92523) c6( ) c7(uK, Ew, bE, bGz, bW, Ls, lb, eX, oj, eV, ajM, bS, fr, ct, bX, Z, y, vO, HO); #c1(HARS) c2(NP_001244970) c3(histidine-tRNA ligase, cytoplasmic isoform 3) c4(92524) c5(92525, 92526, 92527, 92528, 92529, 92530) c6( ) c7(uK, Ew, bE, bGz, bW, Ls, lb, eX, oj, eV, ajM, bS, fr, ct, bX, Z, y, vO, HO); #c1(HARS) c2(NP_001244971) c3(histidine-tRNA ligase, cytoplasmic isoform 4) c4(92531) c5(92532, 92533, 92534, 92535, 92536, 92537) c6( ) c7(uK, Ew, bE, bGz, bW, Ls, lb, eX, oj, eV, ajM, bS, fr, ct, bX, Z, y, vO, HO); #c1(HARS) c2(NP_001276021) c3(histidine-tRNA ligase, cytoplasmic isoform 5) c4(107756, 107757) c5(107758, 107759, 107760, 107761, 107762, 107763, 107764, 107765, 107766, 107767, 107768, 107769) c6( ) c7(uK, Ew, bE, bGz, bW, Ls, lb, eX, oj, eV, ajM, bS, fr, ct, bX, Z, y, vO, HO); #c1(HARS) c2(NP_001276022) c3(histidine-tRNA ligase, cytoplasmic isoform 6) c4(107770) c5(107771, 107772, 107773, 107774, 107775, 107776) c6( ) c7(uK, Ew, bE, bGz, bW, Ls, lb, eX, oj, eV, ajM, bS, fr, ct, bX, Z, y, vO, HO); #c1(HARS) c2(NP_001276023) c3(histidine-tRNA ligase, cytoplasmic isoform 7) c4( ) c5(107777, 107778, 107779, 107780, 107781, 107782) c6( ) c7(uK, Ew, bE, bGz, bW, Ls, lb, eX, oj, eV, ajM, bS, fr, ct, bX, Z, y, vO, HO); #c1(HARS) c2(NP_002100) c3(histidine-tRNA ligase, cytoplasmic isoform 1) c4(124566, 124567) c5(124568, 124569, 124570, 124571, 124572, 124573, 124574) c6( ) c7(uK, Ew, bE, bGz, bW, Ls, lb, eX, oj, eV, ajM, bS, fr, ct, bX, Z, y, vO, HO); #c1(HARS2) c2(NP_001265660) c3(probable histidine-tRNA ligase, mitochondrial isoform 2) c4(99168) c5(99169, 99/70, 99/71, 99/72, 99173, 99174) c6( ) c7(bGA, bW, lb, oj, bX, y, vO, aQe); #c1(HARS2) c2(NP_001265661) c3(probable histidine-tRNA ligase, mitochondrial isoform 3) c4(99175) c5(99/76, 99177, 99178, 99179, 99180, 99181) c6( ) c7(bGA, bW, lb, oj, bX, y, vO, aQe); #c1(HARS2) c2(NP_036340) c3(probable histidine-tRNA ligase, mitochondrial isoform 1 precursor) c4(173077, 173078) c5(173079, 173080, 173081, 173082, 173083, 173084, 173085, 173086, 173087, 173088, 173089, 173090) c6( ) c7(bGA, bW, lb, oj, bX, y, vO, ale); #c1(HARS2) c2(XP_011535922) c3(probable histidine-tRNA ligase, mitochondrial isoform X2) c4(333184, 333185) c5(333186, 333187, 333188, 333189, 333190, 333191, 333192, 333193, 333194, 333195, 333196, 333197) c6( ) c7(bGA, bW, lb, oj, bX, y, vO, aQe); #c1(HAS1) c2(NP_001284365) c3(hyaluronan synthase 1 isoform 2) c4(111831) c5(111832, 111833, 111834, 111835, 111836, 111837) c6( ) c7(T, aw, cz, bB, n, aj, v, j, ci, fd, eA, J, lC, aT, dL, eL, aW, Y, Of, Og, g, t, arY, ac, bE, hL, m, hE, VB); #c1(HAS1) c2(NP_001514) c3(hyaluronan synthase 1 isoform 1) c4(118429) c5(118430, 118431, 118432, 118433, 118434, 118435) c6( ) c7(T, aw, cz, bB, n, aj, v, j, ci, fd, eA, J, lC, aT, dL, eL, aW, Y, Of, Og, g, t, arY, ac, bE, hL, m, hE, VB); #c1(HAS1) c2(XP_011525186) c3(hyaluronan synthase 1 isoform X1) c4(319629, 319630) c5(319631, 319632, 319633, 319634, 319635, 319636, 319637, 319638, 319639, 319640, 319641, 319642) c6( ) c7(T, aw, cz, bB, n, aj, v, j, ci, fd, eA, J, lC, aT, dL, eL, aW, Y, Of, Og, g, t, arY, ac, bE, hL, m, hE, VB); #c1(HAS2) c2(XP_011515304) c3(hyaluronan synthase 2 isoform X1) c4(308175, 308176) c5(308177, 308178, 308179, 308180, 308181, 308182, 308183, 308184, 308185, 308186, 308187, 308188) c6( ) c7(bL, lD, T, dD, wi, ea, fG, acl, fd, v, j, ci, eq, yU, fw, Cb, eA, TI, BY, adG, aT, yZ, dL, K, eL, aW, Y, eH, Of, Og, xv, g, eF, t, f, cm, LB, Ew, at, m, Hb, bGB, lC, bGC, hv); #c1(HAS3) c2(NP_005320) c3(hyaluronan synthase 3 isoform a) c4(154969) c5(154970, 154971, 154972, 154973, 154974, 154975) c6( ) c7(bL, ae, T, cc, bE, ff, hE, lC, aYp, bB, j, ci, m, en, g, t, fq, iu, ad); #c1(HAS3) c2(NP_619515) c3(hyaluronan synthase 3 isoform b) c4(236794) c5(236795, 236796, 236797, 236798, 236799, 236800) c6( ) c7(bL, ae, T, cc, bE, ff, hE, lC, aYp, bB, j, ci, m, en, g, t, fq, iu, ad); #c1(HAT1) c2(NP_003633) c3(histone acetyltransferase type B catalytic subunit) c4(139746, 139747) c5(139748, 139749, 139750, 139751, 139752, 139753, 139754, 139755, 139756, 139757, 139758, 139759) c6( ) c7(aKO, jp); #c1(HAVCR1) c2(NP_001166864) c3(hepatitis A virus cellular receptor 1 precursor) c4(68574, 68575) c5( ) c6( ) c7(bL, bM, T, Dw, fV, jx, hE, cz, fr, pM, V, eh, ai, eA, fU, py, tT, apJ, vw, cy, Ds, h, ji, K, wt, bGD, uQ, iU, sp, hl, eZ, lo, eE, es, cT, iQ, iA, z, MB, E, va, dy, fD, bC, fy, B, y, afX); #c1(HAVCR1) c2(XP_011532816) c3(hepatitis A virus cellular receptor 1 isoform X2) c4( ) c5(328919, 328920, 328921, 328922, 328923, 328924, 328925, 328926, 328927, 328928, 328929, 328930, 328931, 328932, 328933, 328934, 328935, 328936) c6( ) c7(bL, bM, T, Dw, fV, jx, hE, cz, fr, pM, V, eh, ai, eA, fU, py, tT, apJ, vw, cy, Ds, h, ji, K, wt, bGD, uQ, iU, sp, hl, eZ, lo, eE, es, cT, iQ, iA, z, MB, E, va, dy, fD, bC, fy, B, y, afX); #c1(HAVCR1) c2(XP_011532817) c3(hepatitis A virus cellular receptor 1 isoform X3) c4( ) c5(328937, 328938, 328939, 328940, 328941, 328942) c6( ) c7(bL, bM, T, Dw, fV, jx, hE, cz, fr, pM, V, eh, ai, eA, fU, py, tT, apJ, vw, cy, Ds, h, ji, K, wt, bGD, uQ, iU, sp, hl, eZ, lo, eE, es, cT, il, iA, z, MB, E, va, dy, fD, bC, fy, B, y, afX); #c1(HAVCR2) c2(NP_116171) c3(hepatitis A virus cellular receptor 2 precursor) c4(225543) c5(225544, 225545, 225546, 225547, 225548, 225549) c6( ) c7(Kd, T, bM, fM, Dw, if, iW, al, jx, jh, aA, jE, bh, qN, mF, V, eh, hX, xE, apJ, akJ, h, Di, Ds, ji, DY, cp, ul, A, Y, fO, hl, mQ, eZ, fc, lo, vs, aF, iV, bw, bGE, gk, cT, eM, ig, dc, aiM, JM, dW, hE, bt, B, y); #c1(HAX1) c2(NP_001018238) c3(HCLS1-associated protein X-1 isoform b) c4(27522) c5(27523, 27524, 27525, 27526, 27527, 27528) c6( ) c7(gV, bGF, hG, aW, Y, ha, cr, aT, it, uz, afK, oY, K, BG, bw, zA, pb); #c1(HAX1) c2(NP_006109) c3(HCLS1-associated protein X-1 isoform a) c4(161925, 161926) c5(161927, 161928, 161929, 161930, 161931, 161932, 161933, 161934, 161935, 161936, 161937, 161938) c6( ) c7(gV, bGF, hG, aW, Y, ha, cr, aT, it, uz, afK, oY, K, BG, bw, zA, pb); #c1(HBA2) c2(NP_000508) c3(hemoglobin subunit alpha) c4(10693, 10694) c5(10695, 10696, 10697, 10698, 10699, 10700, 10701, 10702, 10703, 10704, 10705, 10706, 10707, 10708, 10709) c6( ) c7(bL, bGQ, bfY, qb, bGl, fN, rf, bGK, aj, QZ, aXA, nA, qN, Rc, bGO, br, J, bGG, oT, yw, BR, oW, dV, h, dz, bGM, qD, alF, on, ZN, A, bGJ, cH, bGL, ih, bfV, bGS, S, avG, aot, bGN, bGH, aFz, bw, lv, dr, ck, ZL, byx, ac, BU, aNv, avS, baO, bGR, qe, wB, gX, bjL, NI, bGP, bfX, dy, xG, ER, pw, wS); #c1(HBB) c2(NP_000509) c3(hemoglobin subunit beta) c4(10710, 10711, 10712) c5(10713, 10714, 10715, 10716, 10717, 10718, 10719, 10720, 10721, 10722, 10723, 10724, 10725, 10726, 10727, 10728, 10729, 10730, 10731, 10732, 10733, 10734, 10735, 10736, 10737, 10738, 10739, 10740, 10741, 10742, 10743, 10744, 10745, 10746, 10747, 10748, 10749, 10750, 10751, 10752, 10753, 10754, 10755, 10756, 10757, 10758, 10759, 10760, 10761, 10762, 10763, 10764, 10765, 10766, 10767, 10768, 10769, 10770, 10771, 10772, 10773, 10774, 10775, 10776, 10777, 10778, 10779, 10780, 10781, 10782, 10783, 10784, 10785, 10786, 10787, 10788, 10789, 10790, 10791, 10792, 10793, 10794, 10795, 10796, 10797, 10798, 10799, 10800, 10801, 10802, 10803, 10804, 10805, 10806, 10807, 10808, 10809, 10810, 10811) c6(10812) c7(T, qo, l, bGZ, ci, bGT, dV, aWa, BZ, qD, byv, aW, bGL, bGX, bGH, avS, nB, NI, uJ, pw, CI, eg, sQ, n, oY, aXA, qN, Rc, bkU, bHb, eh, eW, BR, h, A, pm, ZL, JM, bfX, ER, aDE, DM, rf, cz, QZ, j, C, oT, jZ, aT, bGW, bsd, bGV, bGN, bGU, sS, byx, ac, BU, baO, mG, ao, bfY, qb, jh, Hn, acU, hX, l, F, oW, JK, bHa, bGY, sj, bGJ, bfV, yy, LJ, S, avG, aot, dr, adc, qe, be); #c1(HBD) c2(NP_000510) c3(hemoglobin subunit delta) c4(10813, 10814, 10815, 10816) c5(10817, 10818, 10819, 10820, 10821, 10822, 10823, 10824, 10825, 10826, 10827, 10828, 10829, 10830, 10831, 10832, 10833, 10834) c6( ) c7(T, sS, ZL, BU, qe, avS, bfY, BR, bGN, qb, bfV, JM, NI, bGP, QZ, bfX, pw, aot, qD); #c1(HBE1) c2(NP_005321) c3(hemoglobin subunit epsilon) c4(154976, 154977) c5(154978, 154979, 154980, 154981, 154982, 154983, 154984, 154985, 154986, 154987, 154988, 154989, 154990, 154991, 154992, 154993) c6( ) c7(ck, T, BU, eM, qb, aq, pw, eg, hE, bz, B, hX); #c1(HBEGF) c2(NP_001936) c3(proheparin-binding EGF-like growth factor precursor) c4(122323) c5(122324, 122325, 122326, 122327, 122328, 122329, 122330, 122331, 122332, 122333) c6( ) c7(T, dD, eR, hV, ea, aw, aA, bB, n, eb, j, ci, eq, q, ad, eA, GK, dP, aE, aT, lG, cy, dL, fb, ae, eL, DA, d, JI, aW, vj, ff, az, cr, r, anQ, aq, aF, en, g, t, iK, bz, yX, eK, aAW, uP, kP, lI, bE, wz, E, Hs, va, dy, m, ei, adg, Vj, B); #c1(HBG1) c2(NP_000550) c3(hemoglobin subunit gamma-1) c4(12641) c5(12642, 12643, 12644, 12645, 12646, 12647) c6( ) c7(byD, bHc, fM, bfY, qb, fN, rf, oY, QZ, bHd, ad, BR, oW, By, iY, K, qD, pq, avg, iU, aYp, bGV, avG, aot, dr, ck, ZL, BU, qe, iT, avS, NI, bfX, pw, ER); #c1(HBG2) c2(NP_000175) c3(hemoglobin subunit gamma-2) c4(3817, 3818, 3819) c5(3820, 3821, 3822, 3823, 3824, 3825, 3826, 3827, 3828, 3829, 3830, 3831, 3832, 3833, 3834, 3835, 3836, 3837, 3838, 3839, 3840, 3841) c6( ) c7(ID, qb, aj, bHe, dG, oZ, J, ad, BY, oT, oW, BZ, K, qD, sb, aYp, bGV, aot, dr, lv, bHf, BU, qe, E, pw, ER, lC); #c1(HBM) c2(NP_001003938) c3(hemoglobin subunit mu) c4(20303) c5(20304, 20305, 20306, 20307, 20308, 20309) c6( ) c7(Ta, bHg, Tc); #c1(HBP1) c2(NP_001231191) c3(HMG box-containing protein 1 isoform 1) c4(87234, 87235, 87236, 87237, 87238) c5(87239, 87240, 87241, 87242, 87243, 87244, 87245, 87246, 87247, 87248, 87249, 87250, 87251) c6( ) c7(aW, aT, n, K, ae, ad); #c1(HBP1) c2(NP_036389) c3(HMG box-containing protein 1 isoform 2) c4(173503, 173504) c5(173505, 173506, 173507, 173508, 173509, 173510, 173511, 173512, 173513, 173514, 173515, 173516, 173517, 173518, 173519, 173520, 173521, 173522, 173523, 173524, 173525, 173526, 173527, 173528, 173529, 173530, 173531, 173532, 173533, 173534, 173535, 173536, 173537, 173538, 173539, 173540, 173541) c6( ) c7(aW, aT, n, K, ae, ad); #c1(HBQ1) c2(NP_005322) c3(hemoglobin subunit theta-1) c4(154994) c5(154995, 154996, 154997, 154998, 154999, 155000) c6( ) c7(wS); #c1(HBS1L) c2(NP_001138630) c3(HBS1-like protein isoform 2) c4(57279) c5(57280, 57281, 57282, 57283, 57284, 57285, 57286, 57287, 57288, 57289, 57290, 57291) c6( ) c7(BU, qe, awC, pN, qb, rf, cz, pw, V); #c1(HBS1L) c2(NP_001138679) c3(HBS1-like protein isoform 3) c4(57384) c5(57385, 57386, 57387, 57388, 57389, 57390) c6( ) c7(BU, qe, awC, pN, qb, rf, cz, pw, V); #c1(HBS1L) c2(NP_006611) c3(HBS1-like protein isoform 1) c4(166623, 166624, 166625) c5(166626, 166627, 166628, 166629, 166630, 166631, 166632, 166633, 166634, 166635, 166636, 166637) c6( ) c7(BU, qe, awC, pN, qb, rf, cz, pw, V); #c1(HBZ) c2(NP_005323) c3(hemoglobin subunit zeta) c4(155001) c5(155002, 155003, 155004, 155005, 155006) c6( ) c7(Kd, T, BU, iM, qe, je, qb, kH, Ne, rB, ER); #c1(HBZ) c2(XP_005255344) c3(hemoglobin subunit zeta isoform X2) c4( ) c5(273613) c6( ) c7(Kd, T, BU, iM, qe, je, qb, kH, Ne, rB, ER); #c1(HCAR1) c2(NP_115943) c3(hydroxycarboxylic acid receptor 1) c4(224836) c5(224837, 224838, 224839, 224840, 224841, 224842, 224843, 224844, 224845, 224846, 224847, 224848, 224849, 224850) c6( ) c7(OR, T, zs, aE); #c1(HCAR2) c2(NP_808219) c3(hydroxycarboxylic acid receptor 2) c4(252718) c5(252719, 252720, 252721, 252722, 252723, 252724) c6( ) c7(dD, ff, bE, aW, hk, e, ea, yk, vy, aT, bB, zs, aj, Ej, j, bK, en, eq, cr, J); #c1(HCAR3) c2(NP_006009) c3(hydroxycarboxylic acid receptor 3) c4(161130, 161131) c5(161132, 161133, 161134, 161135, 161136, 161137, 161138, 161139, 161140, 161141, 161142, 161143) c6( ) c7(ka, T, adi, aj, J, kH, aT, bK, EL, af, bmc, d, aW, ff, en, hf, cm, bz, EJ, nB, hk, ki, hR, HB, AN); #c1(HCCS) c2(NP_001116080) c3(cytochrome c-type heme lyase) c4(43404) c5(43405, 43406, 43407, 43408, 43409, 43410, 43411, 43412, 43413, 43414, 43415, 43416, 43417, 43418) c6( ) c7(bL, bM, YU, aj, V, bHh, Hr, J, cy, af, A, kj, Ue, oV, axW, bN, au, aVY, m, fD, eo, bHi, afH); #c1(HCFC1) c2(NP_005325) c3(host cell factor 1) c4(155007) c5(155008, 155009, 155010, 155011, 155012, 155013, 155014, 155015, 155016, 155017, 155018, 155019) c6( ) c7(ck, Fh, bE, Az, eZ, nf, aU, bB, aq, nA, iR, eW, bBm); #c1(HCFC1) c2(XP_006724878) c3(host cell factor 1 isoform X4) c4(296471) c5(296472, 296473, 296474, 296475, 296476, 296477) c6( ) c7(ck, Fh, bE, Az, eZ, nf, aU, bB, aq, nA, iR, eW, bBm); #c1(HCFC1) c2(XP_011529452) c3(host cell factor 1 isoform X9) c4(324889) c5( ) c6( ) c7(ck, Fh, bE, Az, eZ, nf, aU, bB, aq, nA, iR, eW, bBm); #c1(HCFC2) c2(NP_037452) c3(host cell factor 2) c4(175573) c5(175574, 175575, 175576, 175577, 175578, 175579, 175580) c6( ) c7(aq, cd, allo); #c1(HCK) c2(NP_001165601) c3(tyrosine-protein kinase HCK isoform c) c4(67849) c5(67850, 67851, 67852, 67853, 67854, 67855, 67856) c6( ) c7(es, py, Uc, fR, iA, aA, at, h, fd, aF, aDR, k, jj, bt, aDQ); #c1(HCK) c2(NP_001165602) c3(tyrosine-protein kinase HCK isoform d) c4(67857) c5(67858, 67859, 67860, 67861, 67862) c6( ) c7(es, py, Uc, fR, iA, aA, at, h, fd, aF, aDR, k, jj, bt, aDQ); #c1(HCK) c2(NP_001165603) c3(tyrosine-protein kinase HCK isoform e) c4(67863, 67864) c5(67865, 67866, 67867, 67868, 67869) c6( ) c7(es, py, Uc, fR, iA, aA, at, h, fd, af, aDR, k, jj, bt, aDQ); #c1(HCK) c2(NP_001165604) c3(tyrosine-protein kinase HCK isoform b) c4( ) c5(67870, 67871, 67872, 67873, 67874, 67875, 67876, 67877) c6( ) c7(es, py, Uc, fR, iA, aA, at, h, fd, aF, aDR, k, jj, bt, aDQ); #c1(HCK) c2(NP_002101) c3(tyrosine-protein kinase HCK isoform a) c4(124575) c5(124576, 124577, 124578, 124579, 124580, 124581, 124582, 124583, 124584) c6( ) c7(es, py, Uc, fR, iA, aA, at, h, fd, af, aDR, k, jj, bt, aDQ); #c1(HCLS1) c2(NP_005326) c3(hematopoietic lineage cell-specific protein isoform 1) c4(155020) c5(155021, 155022, 155023, 155024, 155025, 155026) c6( ) c7(bO, aA, aj, bX, hF, eA, aR, amW, zn, ec, aCJ, aql, dz, ey, K, hS, zy, H, WZ, jf, eG, LC, Y, Ls, eZ, aF, eE, cm, c, iT, Fi, cj, fl, y, bt, it, L); #c1(HCN1) c2(NP_066550) c3(potassium/sodium hyperpolarization-activated cyclic nucleotide-gated channel 1) c4(207367) c5(207368, 207369, 207370, 207371, 207372, 207373) c6( ) c7(aHS, ha, cV, il, aT, lm, hg, bq, aW, yL, ahm, gu, ir, t, Hv, hq, Cq, cA, HN, Zn, m, zB, cB, gD); #c1(HCN2) c2(NP_001185) c3(potassium/sodium hyperpolarization-activated cyclic nucleotide-gated channel 2) c4( ) c5( ) c6( ) c7(Cq, aHS, ha, hr, dN, cd, Zn, hg, bq, il, gT, gD); #c1(HCN3) c2(NP_065948) c3(potassium/sodium hyperpolarization-activated cyclic nucleotide-gated channel 3) c4(206422) c5(206423, 206424, 206425, 206426, 206427, 206428) c6( ) c7(aHS, an, bq); #c1(HCN3) c2(XP_011545237) c3(potassium/sodium hyperpolarization-activated cyclic nucleotide-gated channel 3 isoform X3) c4( ) c5(344033) c6( ) c7(aHS, an, bq); #c1(HCN4) c2(NP_005468) c3(potassium/sodium hyperpolarization-activated cyclic nucleotide-gated channel 4) c4(156427) c5(156428, 156429, 156430, 156431, 156432, 156433, 156434) c6( ) c7(bHl, cc, mr, bHk, wL, arD, bHm, bHj, bfW, mu); #c1(HCN4) c2(XP_011519450) c3(potassium/sodium hyperpolarization-activated cyclic nucleotide-gated channel 4 isoform X1) c4( ) c5(314124, 314125, 314126, 314127, 314128, 314129) c6( ) c7(bHl, cc, mr, bHk, wL, arD, bHm, bHj, bfW, mu); #c1(HCRT) c2(NP_001515) c3(orexin precursor) c4(118436) c5(118437, 118438, 118439, 118440, 118441, 118442, 118443) c6( ) c7(bL, T, bO, Nt, bB, aj, dG, uu, ct, v, bAM, rc, aR, J, ad, cF, wM, bHq, ds, alX, bHp, bHn, ae, alY, bHo, A, s, cu, Hu, cl, vo, dR, Ye, rR, aBr, cA, Kv, bE, U, hL, bel, abW); #c1(HCRTR2) c2(NP_001517) c3(orexin receptor type 2) c4(118444) c5(118445, 118446, 118447, 118448, 118449, 118450) c6( ) c7(Ye, T, cF, s, yL, Nt, alX, uu, cl, ae, alY, ad); #c1(HCST) c2(NP_001007470) c3(hematopoietic cell signal transducer isoform 2 precursor) c4(22735) c5(22736, 22737, 22738, 22739, 22740, 22741) c6( ) c7(ig, amW, T, K, hX); #c1(HCST) c2(NP_055081) c3(hematopoietic cell signal transducer isoform 1 precursor) c4(178853) c5(178854, 178855, 178856, 178857, 178858, 178859) c6( ) c7(ig, amW, T, K, hX); #c1(HDAC1) c2(XP_011539611) c3(histone deacetylase 1 isoform X1) c4( ) c5(337849, 337850, 337851, 337852, 337853) c6( ) c7(bM, T, Mb, if, bO, aA, bB, n, ti, bkM, fd, ci, qN, PJ, q, ad, eA, hX, dP, eJ, iu, x, F, aE, Db, aT, io, cy, rO, Th, bK, K, h, bq, j, ae, pi, jf, iU, aW, Y, Lf, ff, eZ, aq, kp, aF, en, bu, iR, t, cM, bz, cm, iK, ck, kn, rR, YE, ac, eM, bP, bE, eD, VA, hL, cj, gZ, uJ, k, m, lC, bt, kS, B, y); #c1(HDAC10) c2(NP_001152758) c3(histone deacetylase 10 isoform 2) c4(60157) c5(60158, 60159, 60160, 60161, 60162, 60163) c6( ) c7(av, fM, aq, jE, cm, B); #c1(HDAC10) c2(NP_114408) c3(histone deacetylase 10 isoform 1) c4(222601) c5(222602, 222603, 222604, 222605, 222606, 222607) c6( ) c7(av, fM, aq, jE, cm, B); #c1(HDACH11) c2(NP_001129513) c3(histone deacetylase I1 isoform 2) c4(51818, 51819) c5(51820, 51821, 51822, 51823, 51824) c6( ) c7(T, pc, ex, pg, eV, h, ci); #c1(HDAC11) c2(NP_079103) c3(histone deacetylase I1 isoform 1) c4(217023) c5(217024, 217025, 217026, 217027, 217028, 217029, 217030, 217031) c6( ) c7(T, pc, eX, pg, eV, h, ci); #c1(HDAC11) c2(XP_005265549) c3(histone deacetylase I1 isoform X5) c4( ) c5(279587) c6( ) c7(T, pc, eX, pg, eV, h, ci); #c1(HDAC11) c2(XP_005265550) c3(histone deacetylase I1 isoform X9) c4(279588) c5(279589, 279590, 279591, 279592, 279593) c6( ) c7(T, pc, eX, pg, eV, h, ci); #c1(HDAC11) c2(XP_011532432) c3(histone deacetylase I1 isoform X1) c4( ) c5(328606) c6( ) c7(T, pc, eX, pg, eV, h, ci); #c1(HDAC11) c2(XP_011532433) c3(histone deacetylase 11 isoform X2) c4(328607) c5(328608, 328609, 328610, 328611, 328612) c6( ) c7(T, pc, eX, pg, eV, h, ci); #c1(HDAC11) c2(XP_011532437) c3(histone deacetylase I1 isoform X8) c4( ) c5(328613, 328614, 328615, 328616, 328617) c6( ) c7(T, pc, ex, pg, eV, h, ci); #c1(HDAC2) c2(NP_001518) c3(histone deacetylase 2) c4(118451, 118452) c5(118453, 118454, 118455, 118456, 118457, 118458, 118459, 118460, 118461, 118462, 118463, 118464, 118465, 118466) c6( ) c7(bM, T, aA, bB, n, ti, aj, ci, oH, q, J, ad, eA, dP, iu, aE, Db, aT, kf, vg, vc, h, K, bq, on, De, gj, B, BO, s, Y, ff, r, gl, aq, kp, aF, en, iR, t, cM, bz, cm, hq, TA, bF, ac, iT, bE, CP, at, uJ, OD, m, Th, bt, X); #c1(HDAC2) c2(XP_011534090) c3(histone deacetylase 2 isoform X3) c4(330314) c5(330315, 330316, 330317, 330318, 330319, 330320, 330321, 330322) c6( ) c7(bM, T, aA, bB, n, ti, aj, ci, oH, q, J, ad, eA, dP, iu, aE, Db, aT, kf, vg, vc, h, K, bq, on, De, gj, B, BO, s, Y, ff, r, gl, aq, kp, aF, en, iR, t, cM, bz, cm, hq, TA, bF, ac, iT, bE, CP, at, uJ, OD, m, Th, bt, X); #c1(HDAC3) c2(NP_003874) c3(histone deacetylase 3) c4(14/705) c5(14/706, 141707, 14/708, 141709, 14/710, 14/711) c6( ) c7(T, dD, fM, fR, aZ, ea, jh, aA, bB, n, aj, fd, Kh, ci, aTS, xg, eq, V, eA, J, hX, x, aT, h, K, uV, kl, A, s, JI, rU, dN, aq, lo, aF, iR, t, Vk, cM, bz, Hv, cm, eM, bE, X, cj, gZ, eo, m, ei, y, aDi, B); #c1(HDAC4) c2(NP_006028) c3(histone deacetylase 4) c4(161260, 161261) c5(161262, 161263, 161264, 161265, 161266, 161267, 161268, 161269, 161270, 161271, 161272, 161273, 161274, 161275, 161276, 161277, 161278, 161279, 161280, 161281) c6( ) c7(T, rV, vr, v, nA, C, cd, ad, eW, x, bkm, bri, aql, kf, dL, bl, bq, eL, d, cu, vs, t, nM, ac, c, eM, m, bC, ik, B); #c1(HDAC4) c2(XP_006712940) c3(histone deacetylase 4 isoform X2) c4(286928) c5( ) c6( ) c7(T, rV, vr, v, nA, C, cd, ad, eW, x, bkm, bri, aql, kf, dL, bl, bq, eL, d, cu, vs, t, nM, ac, c, eM, m, bC, ik, B); #c1(HDAC4) c2(XP_011510525) c3(histone deacetylase 4 isoform X6) c4( ) c5(301044, 301045, 301046, 301047, 301048) c6( ) c7(T, rV, vr, v, nA, C, cd, ad, eW, x, bkm, bri, aql, kf, dL, bl, bq, eL, d, cu, vs, t, nM, ac, c, eM, m, bC, ik, B); #c1(HDAC4) c2(XP_011510526) c3(histone deacetylase 4 isoform X7) c4(301049) c5( ) c6( ) c7(T, rV, vr, v, nA, C, cd, ad, eW, x, bkm, bri, aql, kf, dL, bl, bq, eL, d, cu, vs, t, nM, ac, c, eM, m, bC, ik, B); #c1(HDAC4) c2(XP_011510527) c3(histone deacetylase 4 isoform X8) c4(301050) c5( ) c6( ) c7(T, rV, vr, v, nA, C, cd, ad, eW, x, bkm, bri, aql, kf, dL, bl, bq, eL, d, cu, vs, t, nM, ac, c, eM, m, bC, ik, B); #c1(HDAC4) c2(XP_011510529) c3(histone deacetylase 4 isoform X1I) c4( ) c5(301051, 301052) c6( ) c7(T, rV, vr, v, nA, C, cd, ad, eW, x, bkm, bri, aql, kf, dL, bl, bq, eL, d, cu, vs, t, nM, ac, c, eM, m, bC, ik, B); #c1(HDAC4) c2(XP_011510531) c3(histone deacetylase 4 isoform X10) c4(301053) c5(301054, 301055, 301056, 301057, 301058, 301059, 301060, 301061, 301062, 301063, 301064, 301065) c6( ) c7(T, rV, vr, v, nA, C, cd, ad, eW, x, bkm, bri, aql, kf, dL, bl, bq, eL, d, cu, vs, t, nM, ac, c, eM, m, bC, ik, B); #c1(HDAC4) c2(XP_011510532) c3(histone deacetylase 4 isoform X12) c4( ) c5(301066) c6( ) c7(T, rV, vr, v, nA, C, cd, ad, eW, x, bkm, bri, aql, kf, dL, bl, bq, eL, d, cu, vs, t, nM, ac, c, eM, m, bC, ik, B); #c1(HDAC5) c2(XP_005256961) c3(histone deacetylase 5 isoform X2) c4(274612) c5(274613, 274614, 274615, 274616, 274617, 274618) c6( ) c7(ck, x, T, dD, eW, jf, ea, bbY, eH, bO, cm, cu, vs, eq, K, ct, eF, lC, cd); #c1(HDAC5) c2(XP_005256962) c3(histone deacetylase 5 isoform X3) c4(274619) c5( ) c6( ) c7(ck, x, T, dD, eW, jf, ea, bbY, eH, bO, cm, cu, vs, eq, K, ct, eF, lC, cd); #c1(HDAC5) c2(XP_005256963) c3(histone deacetylase 5 isoform X1) c4(274620) c5(274621, 274622, 274623, 274624, 274625, 274626, 274627, 274628, 274629, 274630, 274631, 274632) c6( ) c7(ck, x, T, dD, eW, jf, ea, bbY, eH, bO, cm, cu, vs, eq, K, ct, eF, lC, cd); #c1(HDAC5) c2(XP_011522453) c3(histone deacetylase 5 isoform X7) c4( ) c5(317084) c6( ) c7(ck, x, T, dD, eW, jf, ea, bbY, eH, bO, cm, cu, vs, eq, K, ct, eF, lC, cd); #c1(HDAC6) c2(NP_006035) c3(histone deacetylase 6) c4(161336, 161337, 161338) c5(161339, 161340, 161341, 161342, 161343, 161344, 161345, 161346, 161347, 161348, 161349, 161350, 161351, 161352, 161353, 161354, 161355, 161356, 161357, 161358, 161359, 161360, 161361, 161362, 161363, 161364, 161365, 161366, 161367, 161368, 161369, 161370, 161371, 161372, 161373, 161374, 161375, 161376, 161377, 161378, 161379, 161380, 161381, 161382, 161383, 161384, 161385, 161386, 161387, 161388, 161389, 161390, 161391, 161392, 161393, 161394, 161395, 161396, 161397, 161398, 161399) c6( ) c7(bM, T, fM, FR, Ol, if, ex, jh, aA, cz, n, bB, bHr, C, bX, qN, eA, aR, ad, jz, Db, aT, h, eV, an, ae, aW, Y, ff, aq, aF, en, iR, t, cm, dr, pc, c, eM, bE, hk, ln, FV, U, km, pg, wB, bt, xg, B, ao); #c1(HDAC6) c2(XP_005272621) c3(histone deacetylase 6 isoform X2) c4(284065, 284066, 284067, 284068, 284069, 284070, 284071, 284072) c5(284073, 284074, 284075, 284076, 284077, 284078, 284079, 284080, 284081, 284082, 284083, 284084, 284085, 284086, 284087, 284088) c6( ) c7(bM, T, fM, FR, Ol, if, eX, jh, aA, cz, n, bB, bHr, C, bX, qN, eA, aR, ad, jz, Db, aT, h, eV, an, ae, aW, Y, ff, aq, aF, en, iR, t, cm, dr, pc, c, eM, bE, hk, ln, FV, U, km, pg, wB, bt, xg, B, ao); #c1(HDAC7) c2(NP_001091886) c3(histone deacetylase 7 isoform d) c4(38789) c5(38790, 38791, 38792, 38793, 38794, 38795) c6( ) c7(x, T, aC, aS, m, t); #c1(HDAC7) c2(NP_056216) c3(histone deacetylase 7 isoform a) c4(186456) c5(186457, 186458, 186459, 186460, 186461, 186462, 186463, 186464) c6(186465, 186466) c7(x, T, aC, aS, m, t); #c1(HDAC7) c2(XP_011536780) c3(histone deacetylase 7 isoform X1) c4( ) c5(334456, 334457, 334458, 334459, 334460, 334461) c6( ) c7(x, T, aC, aS, m, t); #c1(HDAC7) c2(XP_011536781) c3(histone deacetylase 7 isoform X3) c4(334462) c5(334463) c6( ) c7(x, T, aC, aS, m, t); #c1(HDAC7) c2(XP_011536784) c3(histone deacetylase 7 isoform X6) c4(334464) c5( ) c6( ) c7(x, T, aC, aS, m, t); #c1(HDAC7) c2(XP_011536785) c3(histone deacetylase 7 isoform X7) c4( ) c5(334465) c6( ) c7(x, T, aC, aS, m, t); #c1(HDAC8) c2(NP_001159890) c3(histone deacetylase 8 isoform 2) c4(64892, 64893) c5(64894, 64895, 64896, 64897, 64898, 64899, 64900, 64901, 64902, 64903, 64904) c6( ) c7(bHt, iM, je, bHs, bw, aNL, aq, nA, iR, cm, B, br); #c1(HDAC8) c2(NP_001159891) c3(histone deacetylase 8 isoform 3) c4(64905) c5(64906, 64907, 64908, 64909, 64910, 64911, 64912, 64913, 64914, 64915, 64916, 64917) c6( ) c7(bHt, iM, je, bHs, bw, aNL, aq, nA, iR, cm, B, br); #c1(HDAC8) c2(NP_001159892) c3(histone deacetylase 8 isoform 4) c4( ) c5(64918, 64919, 64920, 64921, 64922, 64923) c6( ) c7(bHt, iM, je, bHs, bw, aNL, aq, nA, iR, cm, B, br); #c1(HDAC8) c2(NP_001159894) c3(histone deacetylase 8 isoform 5) c4(64924) c5(64925, 64926, 64927, 64928, 64929, 64930) c6( ) c7(bHt, iM, je, bHs, bw, aNL, aq, nA, iR, cm, B, br); #c1(HDAC8) c2(NP_001159920) c3(histone deacetylase 8 isoform 6) c4(64958) c5(64959, 64960, 64961, 64962, 64963, 64964) c6( ) c7(bHt, iM, je, bHs, bw, aNL, aq, nA, iR, cm, B, br); #c1(HDAC8) c2(NP_060956) c3(histone deacetylase 8 isoform 1) c4(198878) c5(198879, 198880, 198881, 198882, 198883, 198884, 198885, 198886) c6( ) c7(bHt, iM, je, bHs, bw, aNL, aq, nA, iR, cm, B, br); #c1(HDAC8) c2(XP_011529288) c3(histone deacetylase 8 isoform X1) c4( ) c5(324722) c6( ) c7(bHt, iM, je, bHs, bw, aNL, aq, nA, iR, cm, B, br); #c1(HDAC8) c2(XP_011529289) c3(histone deacetylase 8 isoform X2) c4(324723, 324724) c5( ) c6( ) c7(bHt, iM, je, bHs, bw, aNL, aq, nA, iR, cm, B, br); #c1(HDAC9) c2(NP_001191073) c3(histone deacetylase 9 isoform 6) c4(80924) c5(80925, 80926, 80927, 80928, 80929, 80930, 80931, 80932, 80933, 80934, 80935, 80936, 80937, 80938, 80939, 80940, 80941, 80942, 80943) c6( ) c7(bM, T, aC, ea, EQ, bO, bB, fd, cL, ci, x, aE, M, K, eL, JI, aW, uY, vs, cm, eM, aCT, U, cj, m, k, avU, de, dD, n, eW, h, eV, dL, pi, Ln, aq, Og, pm, aKv, aDu, cA, hL, pg, fL, B, bL, oe, eX, aA, cz, j, il, q, o, eA, eJ, aT, kf, cy, jj, ae, jf, Uf, oV, Y, r, en, cl, t, iK, ck, kP, c, ac, gX, cS, oX, bt, ha, jh, oH, eq, xg, hF, mC, ad, av, F, io, d, lR, ff, Of, eZ, dq, aF, pT, bz, DF, pc, bE, adg, y, kS, X, us); #c1(HDAC9) c2(NP_001191074) c3(histone deacetylase 9 isoform 7) c4(80944, 80945) c5(80946, 80947, 80948, 80949, 80950) c6( ) c7(bM, T, aC, ea, EQ, bO, bB, fd, cL, ci, x, aE, M, K, eL, JI, aW, uY, vs, cm, eM, aCT, U, cj, m, k, avU, de, dD, n, eW, h, eV, dL, pi, Ln, aq, Og, pm, aKv, aDu, cA, hL, pg, fL, B, bL, oe, eX, aA, cz, j, il, q, o, eA, eJ, aT, kf, cy, jj, ae, jf, Uf, oV, Y, r, en, cl, t, iK, ck, kP, c, ac, gX, cS, oX, bt, ha, jh, oH, eq, xg, hF, mC, ad, av, F, io, d, lR, ff, Of, eZ, dq, aF, pT, bz, DF, pc, bE, adg, y, kS, X, us); #c1(HDAC9) c2(NP_001191075) c3(histone deacetylase 9 isoform 8) c4(80951) c5(80952, 80953, 80954, 80955, 80956, 80957, 80958, 80959, 80960, 80961) c6( ) c7(bM, T, aC, ea, EQ, bO, bB, fd, cL, ci, x, aE, M, K, eL, JI, aW, uY, vs, cm, eM, aCT, U, cj, m, k, avU, de, dD, n, eW, h, eV, dL, pi, Ln, aq, Og, pm, aKv, aDu, cA, hL, pg, fL, B, bL, oe, eX, aA, cz, j, il, q, o, eA, eJ, aT, kf, cy, jj, ae, jf, Uf, oV, Y, r, en, cl, t, iK, ck, kP, c, ac, gX, cS, oX, bt, ha, jh, oH, eq, xg, hF, mC, ad, av, F, io, d, lR, ff, Of, eZ, dq, aF, pT, bz, DF, pc, bE, adg, y, kS, X, us); #c1(HDAC9) c2(NP_001191076) c3(histone deacetylase 9 isoform 9) c4( ) c5(80962, 80963, 80964, 80965, 80966) c6( ) c7(bM, T, aC, ea, EQ, bO, bB, fd, cL, ci, x, aE, M, K, eL, JI, aW, uY, vs, cm, eM, aCT, U, cj, m, k, avU, de, dD, n, eW, h, eV, dL, pi, Ln, aq, Og, pm, aKv, aDu, cA, hL, pg, fL, B, bL, oe, eX, aA, cz, j, il, q, o, eA, eJ, aT, kf, cy, jj, ae, jf, Uf, oV, Y, r, en, cl, t, ik, ck, kP, c, ac, gX, cS, oX, bt, ha, jh, oH, eq, xg, hF, mC, ad, av, F, io, d, lR, ff, Of, eZ, dq, aF, pT, bz, DF, pc, bE, adg, y, kS, X, us); #c1(HDAC9) c2(NP_001191077) c3(histone deacetylase 9 isoform 10) c4(80967) c5(80968, 80969, 80970, 80971, 80972) c6( ) c7(bM, T, aC, ea, EQ, bO, bB, fd, cL, ci, x, aE, M, K, eL, JI, aW, uY, vs, cm, eM, aCT, U, cj, m, k, avU, de, dD, n, eW, h, eV, dL, pi, Ln, aq, Og, pm, aKv, aDu, cA, hL, pg, fL, B, bL, oe, eX, aA, cz, j, il, q, o, eA, eJ, aT, kf, cy, jj, ae, jf, Uf, oV, Y, r, en, cl, t, ik, ck, kP, c, ac, gX, cS, oX, bt, ha, jh, oH, eq, xg, hF, mC, ad, av, F, io, d, lR, ff, Of, eZ, dq, aF, pT, bz, DF, pc, bE, adg, y, kS, X, us); #c1(HDAC9) c2(NP_055522) c3(histone deacetylase 9 isoform 3) c4(181736, 181737, 181738) c5(181739, 181740, 181741, 181742, 181743, 181744, 181745) c6( ) c7(bM, T, aC, ea, EQ, bO, bB, fd, c, ci, x, aE, M, K, eL, JI, aW, uY, vs, cm, eM, aCT, U, cj, m, k, avU, de, dD, n, eW, h, eV, dL, pi, Ln, aq, Og, pm, aKv, aDu, cA, hL, pg, fL, B, bL, oe, eX, aA, cz, j, il, q, o, eA, eJ, aT, kf, cy, jj, ae, jf, Uf, oV, Y, r, en, cl, t, iK, ck, kP, c, ac, gX, cS, oX, bt, ha, jh, oH, eq, xg, hF, mC, ad, av, F, io, d, lR, ff, Of, eZ, dq, aF, pT, bz, DF, pc, bE, adg, y, kS, X, us); #c1(HDAC9) c2(NP_478056) c3(histone deacetylase 9 isoform 1) c4(230849) c5(230850, 230851, 230852, 230853, 230854, 230855, 230856, 230857, 230858, 230859, 230860, 230861) c6( ) c7(bM, T, aC, ea, EQ, bO, bB, fd, cL, ci, x, aE, M, K, eL, JI, aW, uY, vs, cm, eM, aCT, U, cj, m, k, avU, de, dD, n, eW, h, eV, dL, pi, Ln, aq, Og, pm, aKv, aDu, cA, hL, pg, fL, B, bL, oe, eX, aA, cz, j, il, q, o, eA, eJ, aT, kf, cy, jj, ae, jf, Uf, oV, Y, r, en, cl, t, ik, ck, kP, c, ac, gX, cS, oX, bt, ha, jh, oH, eq, xg, hF, mC, ad, av, F, io, d, lR, ff, Of, eZ, dq, aF, pT, bz, DF, pc, bE, adg, y, kS, X, us); #c1(HDAC9) c2(NP_848510) c3(histone deacetylase 9 isoform 4) c4(253746) c5(253747, 253748, 253749, 253750, 253751) c6( ) c7(bM, T, aC, ea, EQ, bO, bB, fd, cL, ci, x, aE, M, K, eL, JI, aW, uY, vs, cm, eM, aCT, U, cj, m, k, avU, de, dD, n, eW, h, eV, dL, pi, Ln, aq, Og, pm, aKv, aDu, cA, hL, pg, fL, B, bL, oe, eX, aA, cz, j, il, q, o, eA, eJ, aT, kf, cy, jj, ae, jf, Uf, oV, Y, r, en, cl, t, ik, ck, kP, c, ac, gX, cS, oX, bt, ha, jh, oH, eq, xg, hF, mC, ad, av, F, io, d, lR, ff, Of, eZ, dq, aF, pT, bz, DF, pc, bE, adg, y, kS, X, us); #c1(HDAC9) c2(NP_848512) c3(histone deacetylase 9 isoform 5) c4(253752) c5(253753, 253754, 253755, 253756, 253757, 253758) c6( ) c7(bM, T, aC, ea, EQ, bO, bB, fd, cL, ci, x, aE, M, K, eL, JI, aW, uY, vs, cm, eM, aCT, U, cj, m, k, avU, de, dD, n, eW, h, eV, dL, pi, Ln, aq, Og, pm, aKv, aDu, cA, hL, pg, fL, B, bL, De, ex, aA, cz, j, il, q, o, eA, eJ, aT, kf, cy, jj, ae, jf, Uf, oV, Y, r, en, cl, t, iK, ck, kP, c, ac, gX, cS, oX, bt, ha, jh, oH, eq, xg, hF, mC, ad, av, F, io, d, IR, ff, Of, eZ, dq, aF, pT, bz, DF, pc, bE, adg, y, kS, X, us); #c1(HDAC9) c2(XP_011513928) c3(histone deacetylase 9 isoform X2) c4( ) c5(306356, 306357, 306358, 306359, 306360, 306361, 306362) c6( ) c7(bM, T, aC, ea, EQ, bO, bB, fd, cL, ci, x, aE, M, K, eL, Jl, aW, uY, vs, cm, eM, aCT, U, cj, m, k, avU, de, dD, n, eW, h, eV, dL, pi, Ln, aq, Og, pm, aKv, aDu, cA, hL, pg, fL, B, bL, oe, eX, aA, cz, j, il, q, o, eA, eJ, aT, kf, cy, jj, ae, jf, Uf, oV, Y, r, en, cl, t, iK, ck, kP, c, ac, gX, cS, oX, bt, ha, jh, oH, eq, xg, hF, mC, ad, av, F, io, d, lR, ff, Of, eZ, dq, aF, pT, bz, DF, pc, bE, adg, y, kS, X, us); #c1(HDAC9) c2(XP_011513931) c3(histone deacetylase 9 isoform X5) c4(306363, 306364) c5(306365) c6( ) c7(bM, T, aC, ea, EQ, bO, bB, fd, cL, ci, x, aE, M, K, eL, JI, aW, uY, vs, cm, eM, aCT, U, cj, m, k, avU, de, dD, n, eW, h, eV, dL, pi, Ln, aq, Og, pm, aKv, aDu, cA, hL, pg, fL, B, bL, oe, eX, aA, cz, j, il, q, o, eA, eJ, aT, kf, cy, jj, ae, jf, Uf, oV, Y, r, en, cl, t, ik, ck, kP, c, ac, gX, cS, oX, bt, ha, jh, oH, eq, xg, hF, mC, ad, av, F, io, d, lR, ff, Of, eZ, dq, aF, pT, bz, DF, pc, bE, adg, y, kS, X, us); #c1(HDAC9) c2(XP_011513936) c3(histone deacetylase 9 isoform X10) c4(306366) c5(306367, 306368, 306369, 306370, 306371, 306372, 306373) c6( ) c7(bM, T, aC, ea, EQ, bO, bB, fd, cL, ci, x, aE, M, K, eL, JI, aW, uY, vs, cm, eM, aCT, U, cj, m, k, avU, de, dD, n, eW, h, eV, dL, pi, Ln, aq, Og, pm, aKv, aDu, cA, hL, pg, FL, B, bL, oe, eX, aA, cz, j, il, q, o, eA, eJ, aT, kf, cy, jj, ae, jf, Uf, oV, Y, r, en, cl, t, iK, ck, kP, c, ac, gX, cS, oX, bt, ha, jh, oH, eq, xg, hF, mC, ad, av, F, io, d, lR, ff, Of, eZ, dq, aF, pT, bz, DF, pc, bE, adg, y, kS, X, us); #c1(HDAC9) c2(XP_011513953) c3(histone deacetylase 9 isoform X18) c4(306374) c5(306375) c6( ) c7(bM, T, aC, ea, EQ, bO, bB, fd, cL, ci, x, aE, M, K, eL, Jl, aW, uY, vs, cm, eM, aCT, U, cj, m, k, avU, de, dD, n, eW, h, eV, dL, pi, Ln, aq, Og, pm, aKv, aDu, cA, hL, pg, fL, B, bL, oe, eX, aA, cz, j, il, q, o, eA, eJ, aT, kf, cy, jj, ae, jf, Uf, oV, Y, r, en, cl, t, ik, ck, kP, c, ac, gX, cS, oX, bt, ha, jh, oH, eq, xg, hF, mC, ad, av, F, io, d, lR, ff, Of, eZ, dq, aF, pT, bz, DF, pc, bE, adg, y, kS, X, us); #c1(HDC) c2(NP_002103) c3(histidine decarboxylase) c4(124591) c5(124592, 124593, 124594, 124595, 124596, 124597) c6( ) c7(bM, T, kh, Dw, aA, ON, asr, rc, baA, cF, bHu, aT, Ds, K, cp, tN, iU, aW, Y, yL, mQ, ff, aF, en, uC, yR, cT, Ta, ac, asq, JM, gv, Dv, Tc); #c1(HDDC2) c2(NP_057147) c3(HD domain-containing protein 2) c4(189103, 189104) c5(189105, 189106, 189107, 189108, 189109, 189110, 189111, 189112, 189113, 189114, 189115, 189116, 189117, 189118, 189119, 189120) c6( ) c7(EQ); #c1(HDGF) c2(NP_001119522) c3(hepatoma-derived growth factor isoform b) c4(44061) c5(44062, 44063, 44064, 44065, 44066, 44067) c6( ) c7(av, T, d, bP, BA, eM, Y, ff, ht, r, bB, n, aq, h, q, en, hu, eA, bz, B, cp); #c1(HDGF) c2(NP_001119523) c3(hepatoma-derived growth factor isoform c) c4(44068, 44069) c5(44070, 44071, 44072, 44073, 44074, 44075, 44076) c6( ) c7(av, T, d, bP, BA, eM, Y, ff, ht, r, bB, n, aq, h, q, en, hu, eA, bz, B, cp); #c1(HDGF) c2(NP_004485) c3(hepatoma-derived growth factor isoform a) c4(146820, 146821) c5(146822, 146823, 146824, 146825, 146826, 146827, 146828, 146829, 146830, 146831, 146832, 146833, 146834, 146835, 146836, 146837) c6( ) c7(av, T, d, bP, BA, eM, Y, ff, ht, r, bB, n, aq, h, q, en, hu, eA, bz, B, cp); #c1(HDGF) c2(XP_011507757) c3(hepatoma-derived growth factor isoform X2) c4( ) c5(297831, 297832, 297833, 297834, 297835) c6( ) c7(av, T, d, bP, BA, eM, Y, ff, ht, r, bB, n, aq, h, q, en, hu, eA, bz, B, cp); #c1(HDGFL1) c2(NP_612641) c3(hepatoma-derived growth factor-like protein 1) c4(236758) c5(236759, 236760, 236761, 236762, 236763, 236764, 236765, 236766, 236767, 236768) c6( ) c7(dY, ac); #c1(HDGFRP3) c2(NP_057157) c3(hepatoma-derived growth factor-related protein 3) c4(189194) c5(189195, 189196, 189197, 189198, 189199, 189200) c6( ) c7(xg); #c1(HDHD1) c2(NP_001129037) c3(pseudouridine-5′-monophosphatase isoform a) c4(50691, 50692) c5(50693, 50694, 50695, 50696, 50697, 50698, 50699, 50700, 50701, 50702, 50703, 50704) c6( ) c7(btu, hQ); #c1(HDHD1) c2(NP_001171606) c3(pseudouridine-5′-monophosphatase isoform c) c4(70758) c5(70759, 70760, 70761, 70762, 70763, 70764) c6( ) c7(btu, hQ); #c1(HDHD1) c2(NP_001171607) c3(pseudouridine-5′-monophosphatase isoform d) c4(70765) c5(70766, 70767, 70768, 70769, 70770, 70771) c6( ) c7(btu, hQ); #c1(HDHD1) c2(NP_036212) c3(pseudouridine-5′-monophosphatase isoform b) c4(172396) c5(172397, 172398, 172399, 172400, 172401, 172402) c6( ) c7(btu, hQ); #c1(HDLBP) c2(NP_001230829) c3(vigilin isoform b) c4(86782, 86783) c5(86784, 86785, 86786, 86787, 86788, 86789, 86790, 86791, 86792, 86793, 86794, 86795, 86796, 86797, 86798, 86799, 86800, 86801, 86802, 86803, 86804, 86805, 86806, 86807, 86808, 86809, 86810, 86811, 86812, 86813, 86814, 86815, 86816, 86817, 86818, 86819, 86820, 86821, 86822, 86823, 86824, 86825, 86826, 86827, 86828, 86829, 86830, 86831, 86832, 86833, 86834, 86835, 86836, 86837, 86838, 86839, 86840) c6( ) c7(T, dD, bri, aW, dc, ea, fZ, aT, hD, eq, ad, bN); #c1(HDLBP) c2(NP_976221) c3(vigilin isoform a) c4(262934) c5(262935, 262936, 262937, 262938, 262939, 262940, 262941, 262942, 262943, 262944, 262945, 262946, 262947, 262948, 262949, 262950, 262951, 262952, 262953, 262954, 262955, 262956, 262957, 262958, 262959, 262960, 262961, 262962, 262963, 262964, 262965, 262966, 262967, 262968, 262969, 262970, 262971, 262972, 262973, 262974, 262975, 262976, 262977, 262978, 262979, 262980, 262981, 262982, 262983, 262984, 262985, 262986, 262987, 262988, 262989, 262990, 262991, 262992) c6( ) c7(T, dD, bri, aW, dc, ea, fZ, aT, hD, eq, ad, bN); #c1(HDLBP) c2(XP_011509363) c3(vigilin isoform X2) c4(299939, 299940, 299941, 299942, 299943, 299944, 299945, 299946, 299947, 299948, 299949, 299950, 299951, 299952) c5( ) c6( ) c7(T, dD, bri, aW, dc, ea, fZ, aT, hD, eq, ad, bN); #c1(HEATR1) c2(NP_060542) c3(HEAT repeat-containing protein 1) c4(196455, 196456, 196457) c5(196458, 196459, 196460, 196461, 196462, 196463, 196464, 196465, 196466, 196467, 196468, 196469, 196470, 196471, 196472, 196473, 196474, 196475) c6( ) c7(aW, aT); #c1(HEATR3) c2(NP_891552) c3(HEAT repeat-containing protein 3) c4(257291) c5(257292, 257293, 257294, 257295, 257296, 257297) c6( ) c7(bIC, iJ); #c1(HEATR5B) c2(NP_061897) c3(HEAT repeat-containing protein 5B) c4(201029, 201030) c5(201031, 201032, 201033, 201034, 201035, 201036, 201037, 201038, 201039, 201040, 201041, 201042, 201043, 201044, 201045, 201046, 201047, 201048) c6( ) c7(aw, eu, e, j, ac); #c1(HEATR5B) c2(XP_005264436) c3(HEAT repeat-containing protein 5B isoform X4) c4(278940) c5( ) c6( ) c7(aw, eu, e, j, ac); #c1(HEATR6) c2(NP_071353) c3(HEAT repeat-containing protein 6) c4(211166, 211167) c5(211168, 211169, 211170, 211171, 211172, 211173, 211174, 211175, 211176, 211177, 211178, 211179) c6( ) c7(T, cK, eM, aW, ds, e, Y, aT, j, ci, cJ, ee, eC); #c1(HEBP1) c2(NP_057071) c3(heme-binding protein 1) c4(188776) c5(188777, 188778, 188779, 188780, 188781, 188782, 188783, 188784) c6( ) c7(dc, T, ad); #c1(HEBP2) c2(NP_055135) c3(heme-binding protein 2) c4(179202) c5(179203, 179204, 179205, 179206, 179207, 179208, 179209, 179210) c6(179211, 179212) c7(aq); #c1(HECA) c2(NP_057301) c3(headcase protein homolog) c4(189978) c5(189979, 189980, 189981, 189982, 189983, 189984) c6( ) c7(T, kh, aW, yL, JM, al, rc); #c1(HECTD2) c2(NP_001271203) c3(probable E3 ubiquitin-protein ligase HECTD2 isoform c) c4(103578, 103579, 103580) c5(103581, 103582, 103583, 103584, 103585, 103586, 103587, 103588, 103589) c6( ) c7(aaF, dt, jV, ae, ad, dS); #c1(HECTD2) c2(NP_775768) c3(probable E3 ubiquitin-protein ligase HECTD2 isoform b) c4(250236) c5(250237, 250238, 250239, 250240, 250241, 250242) c6( ) c7(aaF, dt, jV, ae, ad, dS); #c1(HECTD2) c2(NP_877497) c3(probable E3 ubiquitin-protein ligase HECTD2 isoform a) c4(256879, 256880) c5(256881, 256882, 256883, 256884, 256885, 256886, 256887, 256888, 256889, 256890, 256891) c6( ) c7(aaF, dt, jV, ae, ad, dS); #c1(HECTD2) c2(XP_011537640) c3(probable E3 ubiquitin-protein ligase HECTD2 isoform X2) c4( ) c5(335190, 335191, 335192, 335193, 335194, 335195, 335196, 335197, 335198, 335199) c6( ) c7(aaF, dt, jV, ae, ad, dS); #c1(HECTD4) c2(NP_001103132) c3(probable E3 ubiquitin-protein ligase HECTD4) c4(41838, 41839) c5(41840, 41841, 41842, 41843, 41844, 41845, 41846, 41847, 41848, 41849, 41850) c6( ) c7(bL, ht, De, j, cz); #c1(HECW1) c2(NP_001273988) c3(E3 ubiquitin-protein ligase HECW1 isoform b) c4(105964) c5(105965, 105966, 105967, 105968, 105969, 105970) c6( ) c7(aW, oe, eZ, aT); #c1(HECW1) c2(NP_055867) c3(E3 ubiquitin-protein ligase HECW1 isoform a) c4(184032) c5(184033, 184034, 184035, 184036, 184037, 184038, 184039) c6( ) c7(aW, oe, eZ, aT); #c1(HECW1) c2(XP_011513522) c3(E3 ubiquitin-protein ligase HECW1 isoform X4) c4(305804) c5(305805, 305806, 305807, 305808, 305809, 305810) c6( ) c7(aW, oe, eZ, aT); #c1(HECW2) c2(NP_065811) c3(E3 ubiquitin-protein ligase HECW2 isoform 1) c4(205649, 205650, 205651) c5(205652, 205653, 205654, 205655, 205656, 205657, 205658, 205659, 205660, 205661, 205662, 205663) c6( ) c7(bB, bE, if, qN); #c1(HECW2) c2(XP_006712709) c3(E3 ubiquitin-protein ligase HECW2 isoform X1) c4( ) c5(286812, 286813, 286814, 286815, 286816, 286817) c6( ) c7(bB, bE, if, qN); #c1(HEG1) c2(NP_065784) c3(protein HEG homolog 1 precursor) c4(205428) c5(205429, 205430, 205431, 205432, 205433, 205434) c6( ) c7(t); #c1(HELLS) c2(NP_001275996) c3(lymphoid-specific helicase isoform 2) c4(107691, 107692) c5(107693, 107694, 107695, 107696, 107697, 107698, 107699) c6( ) c7(T, kl, eU, eM, xR, qt, aW, r, aT, aA, aF, m, iK, q); #c1(HELLS) c2(NP_001275997) c3(lymphoid-specific helicase isoform 3) c4( ) c5(107700) c6( ) c7(T, kl, eU, eM, xR, qt, aW, r, aT, aA, aF, m, iK, q); #c1(HELLS) c2(NP_001275999) c3(lymphoid-specific helicase isoform 5) c4(107701, 107702, 107703, 107704) c5(107705, 107706, 107707, 107708, 107709, 107710) c6( ) c7(T, kl, eU, eM, xR, qt, aW, r, aT, aA, aF, m, iK, q); #c1(HELLS) c2(NP_001276001) c3(lymphoid-specific helicase isoform 7) c4( ) c5(107712, 107713, 107714, 107715, 107716, 107717, 107718, 107719, 107720, 107721, 107722, 107723, 107724, 107725, 107726, 107727, 107728, 107729, 107730, 107731, 107732, 107733) c6( ) c7(T, kl, eU, eM, xR, qt, aW, r, aT, aA, aF, m, iK, q); #c1(HELLS) c2(NP_001276002) c3(lymphoid-specific helicase isoform 8) c4( ) c5(107734) c6( ) c7(T, kl, eU, eM, xR, qt, aW, r, aT, aA, aF, m, iK, q); #c1(HELLS) c2(NP_060533) c3(lymphoid-specific helicase isoform 1) c4(196441) c5(196442, 196443, 196444, 196445, 196446, 196447) c6( ) c7(T, kl, eU, eM, xR, qt, aW, r, aT, aA, aF, m, iK, q); #c1(HELQ) c2(NP_598375) c3(helicase POLQ-like isoform 1) c4(235457) c5(235458, 235459, 235460, 235461, 235462, 235463) c6( ) c7(hy, gv, m, bX, t, iK, it); #c1(HELT) c2(NP_001287710) c3(hairy and enhancer of split-related protein HELT isoform 1) c4(112592, 112593) c5(112594, 112595, 112596, 112597, 112598, 112599, 112600, 112601, 112602, 112603, 112604, 112605) c6( ) c7(uL, bxg, ey, dk); #c1(HELT) c2(NP_001287711) c3(hairy and enhancer of split-related protein HELT isoform 2) c4(112606) c5(112607, 112608, 112609, 112610, 112611, 112612) c6( ) c7(uL, bxg, ey, dk); #c1(HELZ) c2(NP_055692) c3(probable helicase with zinc finger domain) c4(182797) c5(182798, 182799, 182800, 182801, 182802, 182803) c6( ) c7(v, T, fq, V); #c1(HELZ) c2(XP_005257946) c3(probable helicase with zinc finger domain isoform X3) c4(275435) c5( ) c6( ) c7(v, T, fq, V); #c1(HELZ) c2(XP_005257947) c3(probable helicase with zinc finger domain isoform X4) c4( ) c5(275436, 275437, 275438, 275439, 275440, 275441) c6( ) c7(v, T, fq, V); #c1(HELZ2) c2(NP_001032412) c3(helicase with zinc finger domain 2 isoform 1) c4(31332) c5(31333, 31334, 31335, 31336, 31337, 31338, 31339) c6( ) c7(dl, v); #c1(HELZ2) c2(NP_208384) c3(helicase with zinc finger domain 2 isoform 2) c4(228547) c5(228548, 228549, 228550, 228551, 228552) c6( ) c7(dQ, v); #c1(HEMGN) c2(XP_011517148) c3(hemogen isoform X1) c4(310690) c5(310691, 310692, 310693, 310694, 310695, 310696, 310697, 310698, 310699, 310700) c6( ) c7(ck, Na, hF, Nc, j, Mu, bt); #c1(HEPACAM) c2(NP_689935) c3(hepatocyte cell adhesion molecule precursor) c4(244389) c5(244390, 244391, 244392, 244393, 244394, 244395) c6( ) c7(XX, es, T, d, aPn, id, iA, aW, aT, By, bHv, bHw, K, bHx, nA, cb, dL, fr, eL, B, bN); #c1(HEPACAM) c2(XP_005271506) c3(hepatocyte cell adhesion molecule isoform X3) c4(283432) c5(283433, 283434, 283435, 283436, 283437, 283438) c6( ) c7(XX, es, T, d, aPn, id, iA, aW, aT, By, bHv, bHw, K, bHx, nA, cb, dL, fr, eL, B, bN); #c1(HEPH) c2(NP_001124332) c3(hephaestin isoform c precursor) c4(48704) c5( ) c6( ) c7(yw, hl, oW, r, yy, yu, uu, aln, q); #c1(HEPH) c2(NP_001269070) c3(hephaestin isoform d precursor) c4(100357) c5(100358, 100359, 100360, 100361, 100362, 100363, 100364, 100365, 100366, 100367, 100368, 100369) c6( ) c7(yw, hl, oW, r, yy, yu, uu, aln, q); #c1(HEPH) c2(NP_055614) c3(hephaestin isoform b) c4(182294) c5(182295, 182296, 182297, 182298, 182299) c6( ) c7(yw, hl, oW, r, yy, yu, uu, aln, q); #c1(HEPH) c2(NP_620074) c3(hephaestin isoform a) c4(237276, 237277, 237278) c5(237279, 237280, 237281, 237282, 237283, 237284, 237285, 237286) c6( ) c7(yw, hl, oW, r, yy, yu, uu, aln, q); #c1(HEPH) c2(XP_006724785) c3(hephaestin isoform X2) c4( ) c5(296325, 296326, 296327, 296328, 296329, 296330, 296331, 296332, 296333, 296334, 296335) c6( ) c7(yw, hl, oW, r, yy, yu, uu, aln, q); #c1(HEPH) c2(XP_011529375) c3(hephaestin isoform X1) c4( ) c5(324757, 324758, 324759, 324760, 324761, 324762) c6( ) c7(yw, hl, oW, r, yy, yu, uu, aln, q); #c1(HEPH) c2(XP_011529377) c3(hephaestin isoform X4) c4(324763, 324764) c5(324765, 324766, 324767, 324768, 324769, 324770) c6( ) c7(yw, hl, oW, r, yy, yu, uu, aln, q); #c1(HEPN1) c2(NP_001032647) c3(putative cancer susceptibility gene HEPN1 protein) c4(31404) c5(31405, 31406, 31407, 31408, 31409, 31410) c6( ) c7(yo, OG, B); #c1(HERC1) c2(NP_003913) c3(probable E3 ubiquitin-protein ligase HERC1) c4(142115, 142116) c5(142117, 142118, 142119, 142120, 142121, 142122) c6( ) c7(agl, nA); #c1(HERC1) c2(XP_011520442) c3(probable E3 ubiquitin-protein ligase HERC1 isoform X1) c4( ) c5(315323, 315324, 315325, 315326, 315327, 315328, 315329, 315330, 315331, 315332, 315333, 315334) c6( ) c7(agl, nA); #c1(HERC1) c2(XP_011520445) c3(probable E3 ubiquitin-protein ligase HERC1 isoform X4) c4( ) c5(315335, 315336, 315337, 315338, 315339) c6( ) c7(agl, nA); #c1(HERC1) c2(XP_011520448) c3(probable E3 ubiquitin-protein ligase HERC1 isoform X7) c4( ) c5(315340, 315341, 315342, 315343, 315344, 315345) c6( ) c7(agl, nA); #c1(HERC1) c2(XP_011520453) c3(probable E3 ubiquitin-protein ligase HERC1 isoform X12) c4(315346) c5( ) c6( ) c7(agl, nA); #c1(HERC1) c2(XP_011520454) c3(probable E3 ubiquitin-protein ligase HERC1 isoform X13) c4(315347, 315348) c5( ) c6( ) c7(agl, nA); #c1(HERC2) c2(NP_004658) c3(E3 ubiquitin-protein ligase HERC2) c4(148581) c5(148582, 148583, 148584, 148585, 148586, 148587, 148588) c6( ) c7(bHz, bM, iV, A, hk, Y, bHB, agl, ge, fe, nA, bHy, cm, bHA, rc); #c1(HERC2) c2(XP_011520433) c3(E3 ubiquitin-protein ligase HERC2 isoform X4) c4(315317) c5( ) c6( ) c7(bHz, bM, iV, A, hk, Y, bHB, agl, ge, fe, nA, bHy, cm, bHA, rc); #c1(HERC2) c2(XP_011520439) c3(E3 ubiquitin-protein ligase HERC2 isoform X10) c4( ) c5(315318, 315319, 315320, 315321, 315322) c6( ) c7(bHz, bM, iV, A, hk, Y, bHB, agl, ge, fe, nA, bHy, cm, bHA, rc); #c1(HERC3) c2(NP_001258531) c3(probable E3 ubiquitin-protein ligase HERC3 isoform 2) c4(96110) c5( ) c6( ) c7(aq); #c1(HERC3) c2(NP_055421) c3(probable E3 ubiquitin-protein ligase HERC3 isoform 1) c4(181043, 181044, 181045, 181046) c5(181047, 181048, 181049, 181050, 181051, 181052, 181053, 181054, 181055, 181056, 181057, 181058, 181059, 181060, 181061, 181062) c6( ) c7(aq); #c1(HERC3) c2(XP_005263386) c3(probable E3 ubiquitin-protein ligase HERC3 isoform X2) c4(278468, 278469) c5( ) c6( ) c7(aq); #c1(HERC5) c2(NP_057407) c3(E3 ISG15-protein ligase HERC5) c4(190589) c5(190590, 190591, 190592, 190593, 190594, 190595) c6( ) c7(ck, bM, dP, Bv, ds, h, gA, aAE); #c1(HERC6) c2(NP_001158608) c3(probable E3 ubiquitin-protein ligase HERC6 isoform 2) c4(63706) c5(63707, 63708, 63709, 63710, 63711, 63712, 63713, 63714, 63715, 63716, 63717, 63718, 63719, 63720, 63721, 63722, 63723, 63724, 63725, 63726, 63727, 63728) c6( ) c7(ds); #c1(HERC6) c2(NP_060382) c3(probable E3 ubiquitin-protein ligase HERC6 isoform 1) c4(195712, 195713, 195714, 195715) c5(195716, 195717, 195718, 195719, 195720, 195721) c6( ) c7(ds); #c1(HERC6) c2(XP_005263140) c3(probable E3 ubiquitin-protein ligase HERC6 isoform X1) c4( ) c5(278339, 278340) c6( ) c7(ds); #c1(HERPUD1) c2(NP_001010989) c3(homocysteine-responsive endoplasmic reticulum-resident ubiquitin-like domain member 1 protein isoform 2) c4(24468, 24469) c5(24470, 24471, 24472, 24473, 24474, 24475, 24476, 24477, 24478, 24479, 24480) c6( ) c7(T, id, e, jh, sM, bX, ad, x, kH, h, dL, K, ox, eL, je, ck, iM, W, wE, k, bC, ei); #c1(HERPUD1) c2(NP_055500) c3(homocysteine-responsive endoplasmic reticulum-resident ubiquitin-like domain member I protein isoform 1) c4(181568) c5(181569, 181570, 181571, 181572, 181573) c6( ) c7(T, id, e, jh, sM, bX, ad, x, kH, h, dL, K, ox, eL, je, ck, iM, W, wE, k, bC, ei); #c1(HERPUD1) c2(XP_006721415) c3(homocysteine-responsive endoplasmic reticulum-resident ubiquitin-like domain member 1 protein isoform X1) c4(293994) c5(293995, 293996, 293997, 293998, 293999, 294000, 294001, 294002) c6( ) c7(T, id, e, jh, sM, bX, ad, x, kH, h, dL, K, ox, eL, je, ck, iM, W, wE, k, bC, ei); #c1(HES1) c2(NP_005515) c3(transcription factor HES-1) c4(157008) c5(157009, 157010, 157011, 157012, 157013, 157014, 157015) c6( ) c7(Fv, T, os, fM, aC, iB, if, ha, aA, bB, n, axk, v, oZ, ci, il, Fc, q, ad, eA, ip, iC, bv, av, ft, jz, aE, x, ahL, kH, aT, bl, Tj, fb, ae, Qe, jf, axl, A, d, B, s, bf, As, aW, Y, iU, eH, cr, r, nY, hN, aq, aF, g, cl, as, t, eF, bz, cm, iV, ck, iD, c, eM, bE, E, m, be, bt, X, BA); #c1(HES2) c2(XP_011539915) c3(transcription factor HES-2 isoform X1) c4(338197, 338198) c5(338199, 338200, 338201, 338202, 338203, 338204, 338205, 338206, 338207, 338208, 338209, 338210, 338211, 338212, 338213, 338214) c6( ) c7(cm); #c1(HES6) c2(NP_001136325) c3(transcription cofactor HES-6 isoform b) c4(54812) c5(54813, 54814, 54815, 54816, 54817, 54818, 54819) c6( ) c7(bv, gk, T, QG, cA, bE, aW, oV, al, aT, ci, eA, Xu, Fc, B); #c1(HES6) c2(NP_061115) c3(transcription cofactor HES-6 isoform a) c4(199165, 199(66) c5(199167, 199168, 199169, 199170, 199171, 199172, 199173, 199174, 199175, 199176, 199177, 199178) c6( ) c7(bv, gk, T, QG, cA, bE, aW, oV, al, aT, ci, eA, Xu, Fc, B); #c1(HES7) c2(NP_001159439) c3(transcription factor HES-7 isoform 1) c4( ) c5(64051, 64052, 64053, 64054, 64055) c6( ) c7(qb, bfU, bBk, Al, bHC); #c1(HES7) c2(NP_115969) c3(transcription factor HES-7 isoform 2) c4(224985) c5(224986, 224987, 224988, 224989, 224990, 224991, 224992) c6( ) c7(qb, bfU, bBk, Al, bHC); #c1(HES7) c2(XP_011522340) c3(transcription factor HES-7 isoform X1) c4(317017) c5(317018) c6( ) c7(qb, bfU, bBk, Al, bHC); #c1(HESX1) c2(NP_003856) c3(homeobox expressed in ES cells 1) c4(141538, 141539) c5(141540, 141541, 141542, 141543, 141544, 141545, 141546, 141547, 141548, 141549, 141550, 141551, 141552) c6( ) c7(bBJ, bHD, acz, bHE, tT, cz, v, y, afx, bHF, yn); #c1(HEXA) c2(NP_000511) c3(beta-hexosaminidase subunit alpha preproprotein) c4(10835, 10836, 10837) c5(10838, 10839, 10840, 10841, 10842, 10843, 10844, 10845, 10846, 10847, 10848, 10849, 10850, 10851, 10852, 10853, 10854, 10855, 10856) c6( ) c7(OI, bDa, jh, nA, arX, eA, bCZ, bDz, aV, bzk, kf, aU, h, an, apl, xi, dq, aYR, dr, ck, mO, rR, bHG, ky, aDA, bHl, U, Lp, awM, bHH, Lq, ati, y, ao); #c1(HEXB) c2(NP_000512) c3(beta-hexosaminidase subunit beta isoform 1 preproprotein) c4(10857, 10858) c5(10859, 10860, 10861, 10862, 10863, 10864, 10865, 10866) c6( ) c7(bM, gk, mO, bHJ, bHL, lR, bHM, zn, Ol, bDa, aDA, al, awM, h, E, Lq, ey, bHK, y, eA, bHN); #c1(HEXB) c2(NP_001278933) c3(beta-hexosaminidase subunit beta isoform 2) c4( ) c5(111070, 111071, 111072, 111073, 111074) c6( ) c7(bM, gk, mO, bHJ, bHL, lR, bHM, zn, OI, bDa, aDA, al, awM, h, E, Lq, ey, bHK, y, eA, bHN); #c1(HEXDC) c2(NP_775891) c3(hexosaminidase D) c4(250583) c5(250584, 250585, 250586, 250587, 250588, 250589) c6( ) c7(bDa, awM, aDA, U); #c1(HEXDC) c2(XP_005256404) c3(hexosaminidase D isoform X4) c4(274/77) c5(274/78, 274179, 274180, 274181, 274182, 274183) c6( ) c7(bDa, awM, aDA, U); #c1(HEXDC) c2(XP_011521863) c3(hexosaminidase D isoform X3) c4( ) c5(316473) c6( ) c7(bDa, awM, aDA, U); #c1(HEXIM1) c2(NP_006451) c3(protein HEXIM1) c4(165326) c5(165327, 165328, 165329, 165330, 165331, 165332) c6( ) c7(ck, ly, T, lD, jW, iT, Ae, lC, oj, Ak, cm, it, Fb, QK, bt, ae, B, ad, (x); #c1(HEY1) c2(NP_001035798) c3(hairy/enhancer-of-split related with YRPW motif protein 1 isoform b) c4(33112) c5(33113, 33114, 33115, 33116, 33117, 33118, 33119, 33120, 33121, 33122) c6( ) c7(ck, ae, av, kh, d, VX, Tb, jf, fG, aT, cz, apy, uU, Fb, fr, yq, as, Fc, bz, ad, BA); #c1(HEY1) c2(NP_036390) c3(hairy/enhancer-of-split related with YRPW motif protein 1 isoform a) c4(173542) c5(173543, 173544, 173545, 173546, 173547, 173548, 173549) c6( ) c7(ck, ae, av, kh, d, VX, Tb, jf, fG, aT, cz, apy, uU, Fb, fr, yq, as, Fc, bz, ad, BA); #c1(HEY2) c2(NP_036391) c3(hairy/enhancer-of-split related with YRPW motif protein 2) c4(173550, 173551) c5(173552, 173553, 173554, 173555, 173556, 173557, 173558) c6( ) c7(T, acl, sM, ajM, j, ci, MT, ad, md, iC, Mg, Ot, aT, h, vc, uV, aVl, rz, ae, qw, bf, aVJ, aW, as, hJ, f, sx, acw, gY, wE, bD, BA); #c1(HEY2) c2(XP_011533951) c3(hairy/enhancer-of-split related with YRPW motif protein 2 isoform X1) c4( ) c5(330148) c6( ) c7(T, acl, sM, ajM, j, ci, MT, ad, md, iC, Mg, Ot, aT, h, vc, uV, aVl, rz, ae, qw, bf, aVJ, aW, as, hJ, f, sx, acw, gY, wE, bD, BA); #c1(HEY2) c2(XP_011533952) c3(hairy/enhancer-of-split related with YRPW motif protein 2 isoform X3) c4( ) c5(330149, 330150, 330151, 330152, 330153) c6( ) c7(T, acl, sM, ajM, j, ci, MT, ad, md, iC, Mg, Ot, aT, h, vc, uV, aVl, rz, ae, qw, bf, aVJ, aW, as, hJ, f, sx, acw, gY, wE, bD, BA); #c1(HFE) c2(NP_000401) c3(hereditary hemochromatosis protein isoform 1 precursor) c4(8544, 8545) c5(8546, 8547, 8548, 8549, 8550, 8551) c6( ) c7(oO, atT, T, fR, ea, if, e, al, fd, bB, cL, Vd, ci, bHU, aY, ip, x, aEQ, bHS, dY, aE, rw, Gb, M, dV, K, bHT, wt, dQ, qD, dF, N, aiJ, tw, aa, hm, zQ, pr, aW, adw, az, bGL, hl, LZ, fc, bHW, alb, g, as, cm, bw, bHP, wT, bwQ, eY, aBr, rq, aNv, iT, aGe, mx, U, cj, vy, Gs, cX, dW, QF, k, m, abX, pw, dD, gU, aw, ht, oY, aj, fr, aUX, bHO, cd, br, ai, bbB, cc, hw, pN, Bj, aMD, h, eV, dL, ZN, A, sq, aq, pm, mo, bHR, Zj, Wt, op, vU, GG, cO, mg, at, eo, aDE, B, bL, bGu, jv, pF, eR, kA, oe, jx, eX, aA, cz, QZ, j, v, bX, Qo, V, q, J, eA, UE, yw, uT, aT, yu, dz, jj, an, bau, ae, af, eu, kl, alX, s, aDf, atl, r, lo, rs, eS, sK, xw, el, aDQ, ck, BU, rR, ac, c, VJ, wg, aUW, bbA, hr, mf, cS, aDR, gT, bt, ao, vi, Kd, am, qX, qb, uG, jh, aHv, uu, jE, fu, eq, hF, ad, dZ, kL, qC, fU, hX, wD, F, oz, sO, tT, qh, oW, bHQ, mu, uX, VX, BO, et, cH, Fm, bHX, eZ, yy, bHV, aF, hp, S, aot, iR, dr, ek, D, gk, Bv, qe, pc, bE, ag, QV, ll, gZ, cP, ei, hE, aed, alq, y); #c1(HFE) c2(NP_001287678) c3(hereditary hemochromatosis protein isoform 12 precursor) c4(112528, 112529) c5(112530, 112531, 112532, 112533, 112534, 112535, 112536) c6( ) c7(oO, atT, T, fR, ea, if, e, al, fd, bB, cL, Vd, ci, bHU, aY, ip, x, aEQ, bHS, dY, aE, rw, Gb, M, dV, K, bHT, wt, dQ, qD, dF, N, aiJ, tw, aa, hm, zQ, pr, aW, adw, az, bGL, hl, LZ, fc, bHW, alb, g, as, cm, bw, bHP, wT, bwQ, eY, aBr, rq, aNv, iT, aGe, mx, U, cj, vy, Gs, cx, dW, QF, k, m, abX, pw, dD, gU, aw, ht, oY, aj, fr, aUX, bHO, cd, br, ai, bbB, cc, hw, pN, Bj, aMD, h, eV, dL, ZN, A, sq, aq, pm, mo, bHR, Zj, Wt, op, vU, GG, cO, mg, at, eo, aDE, B, bL, bGu, jv, pF, eR, kA, oe, jx, eX, aA, cz, QZ, j, v, bX, Qo, V, q, J, eA, UE, yw, uT, aT, yu, dz, jj, an, bau, ae, af, eu, kl, alX, s, aDf, atl, r, lo, rs, eS, sK, xw, el, aDQ, ck, BU, rR, ac, c, VJ, wg, aUW, bbA, hr, mf, cS, aDR, gT, bt, ao, vi, Kd, am, qX, qb, uG, jh, aHv, uu, jE, fu, eq, hF, ad, dZ, kL, qC, fU, hX, wD, F, oz, sO, tT, qh, oW, bHQ, mu, uX, VX, BO, et, cH, Fm, bHX, eZ, yy, bHV, aF, hp, S, aot, iR, dr, ek, D, gk, Bv, qe, pc, bE, ag, QV, ll, gZ, cP, ei, hE, aed, alq, y); #c1(HFE) c2(NP_620572) c3(hereditary hemochromatosis protein isoform 3 precursor) c4(237716) c5(237717, 237718, 237719, 237720, 237721, 237722) c6( ) c7(oO, atT, T, fR, ea, if, e, al, fd, bB, cL, Vd, ci, bHU, aY, ip, x, aEQ, bHS, dY, aE, rw, Gb, M, dV, K, bHT, wt, dQ, qD, dF, N, aiJ, tw, aa, hm, zQ, pr, aW, adw, az, bGL, hl, LZ, fc, bHW, alb, g, as, cm, bw, bHP, wT, bwQ, eY, aBr, rq, aNv, iT, aGe, mx, U, cj, vy, Gs, cx, dW, QF, k, m, abX, pw, dD, gU, aw, ht, oY, aj, fr, aUX, bHO, cd, br, ai, bbB, cc, hw, pN, Bj, aMD, h, eV, dL, ZN, A, sq, aq, pm, mo, bHR, Zj, Wt, op, vU, GG, cO, mg, at, eo, aDE, B, bL, bGu, jv, pF, eR, KA, oe, jx, eX, aA, cz, AZ, j, v, bX, Qo, V, q, J, eA, UE, yw, uT, aT, yu, dz, jj, an, bau, ae, af, eu, kl, alX, s, aDf, atl, r, lo, rs, eS, sK, xw, el, aDQ, ck, BU, rR, ac, c, VJ, wg, aUW, bbA, hr, mf, cS, aDR, gT, bt, ao, vi, Kd, am, qX, qb, uG, jh, aHv, uu, jE, fu, eq, hF, ad, dZ, kL, qC, fU, hX, wD, F, oz, sO, tT, qh, oW, bHQ, mu, uX, VX, BO, et, cH, Fm, bHX, eZ, yy, bHV, aF, hp, S, aot, iR, dr, ek, D, gk, Bv, qe, pc, bE, ag, QV, ll, gZ, cP, ei, hE, aed, alq, y); #c1(HFE) c2(NP_620573) c3(hereditary hemochromatosis protein isoform 4 precursor) c4(237723) c5(237724, 237725, 237726, 237727, 237728, 237729) c6( ) c7(oO, atT, T, fR, ea, if, e, al, fd, bB, cL, Vd, ci, bHU, aY, ip, x, aEQ, bHS, dY, aE, rw, Gb, M, dV, K, bHT, wt, dQ, qD, dF, N, aiJ, tw, aa, hm, zQ, pr, aW, adw, az, bGL, hl, LZ, fc, bHW, alb, g, as, cm, bw, bHP, wT, bwQ, eY, aBr, rq, aNv, iT, aGe, mx, U, cj, vy, Gs, cX, dW, QF, k, m, abX, pw, dD, gU, aw, ht, oY, aj, fr, aUX, bHO, cd, br, ai, bbB, cc, hw, pN, Bj, aMD, h, eV, dL, ZN, A, sq, aq, pm, mo, bHR, Zj, Wt, op, vU, GG, cO, mg, at, eo, aDE, B, bL, bGu, jv, pF, eR, kA, oe, jx, eX, aA, cz, QZ, j, v, bX, Qo, V, q, J, eA, UE, yw, uT, aT, yu, dz, jj, an, bau, ae, af, eu, kl, alX, s, aDf, atl, r, lo, rs, eS, sK, xw, el, aDQ, ck, BU, rR, ac, c, VJ, wg, aUW, bbA, hr, mf, cS, aDR, gT, bt, ao, vi, Kd, am, qX, qb, uG, jh, aHv, uu, jE, fu, eq, hF, ad, dZ, kL, qC, fU, hX, wD, F, oz, sO, tT, qh, oW, bHQ, mu, uX, VX, BO, et, cH, Fm, bHX, eZ, yy, bHV, aF, hp, S, aot, iR, dr, ek, D, gk, Bv, qe, pc, bE, ag, QV, ll, gZ, cP, ei, hE, aed, alq, y); #c1(HFE) c2(NP_620575) c3(hereditary hemochromatosis protein isoform 6 precursor) c4(237730) c5(237731, 237732, 237733, 237734, 237735, 237736, 237737, 237738, 237739, 237740, 237741, 237742) c6( ) c7(oO, atT, T, fR, ea, if, e, al, fd, bB, cL, Vd, ci, bHU, aY, ip, x, aEQ, bHS, dY, aE, rw, Gb, M, dV, K, bHT, wt, da, qD, dF, N, aiJ, tw, aa, hm, zQ, pr, aW, adw, az, bGL, hl, LZ, fc, bHW, alb, g, as, cm, bw, bHP, wT, bwQ, eY, aBr, rq, aNv, iT, aGe, mx, U, cj, vy, Gs, cX, dW, QF, k, m, abX, pw, dD, gU, aw, ht, oY, aj, fr, aUX, bHO, cd, br, ai, bbB, cc, hw, pN, Bj, aMD, h, eV, dL, ZN, A, sq, aq, pm, mo, bHR, Zj, Wt, op, vU, GG, cO, mg, at, eo, aDE, B, bL, bGu, jv, pF, eR, kA, oe, jx, eX, aA, cz, AZ, j, v, bX, Qo, V, q, J, eA, UE, yw, uT, aT, yu, dz, jj, an, bau, ae, af, eu, kl, alX, s, aDf, atl, r, lo, rs, eS, sK, xw, el, aDQ, ck, BU, rR, ac, c, VJ, wg, aUW, bbA, hr, mf, cS, aDR, gT, bt, ao, vi, Kd, am, qX, qb, uG, jh, aHv, uu, jE, fu, eq, hF, ad, dZ, kL, qC, fU, hX, wD, F, oz, sO, tT, qh, oW, bHQ, mu, ux, VX, BO, et, cH, Fm, bHX, eZ, yy, bHV, aF, hp, S, aot, iR, dr, ek, D, gk, Bv, qe, pc, bE, ag, QV, Il, gZ, cP, ei, hE, aed, alq, y); #c1(HFE) c2(NP_620576) c3(hereditary hemochromatosis protein isoform 7 precursor) c4(237743) c5(237744, 237745, 237746, 237747, 237748, 237749) c6( ) c7(oO, atT, T, fR, ea, if, e, al, fd, bB, cL, Vd, ci, bHU, aY, ip, x, aEQ, bHS, dY, aE, rw, Gb, M, dV, K, bHT, wt, dQ, qD, dF, N, aiJ, tw, aa, hm, zQ, pr, aW, adw, az, bGL, hl, LZ, fc, bHW, alb, g, as, cm, bw, bHP, wT, bwQ, eY, aBr, rq, aNv, iT, aGe, mx, U, cj, vy, Gs, cx, dW, QF, k, m, abX, pw, dD, gU, aw, ht, oY, aj, fr, aUX, bHO, cd, br, ai, bbB, cc, hw, pN, Bj, aMD, h, eV, dL, ZN, A, sq, aq, pm, mo, bHR, Zj, Wt, op, vU, GG, cO, mg, at, eo, aDE, B, bL, bGu, jv, pF, eR, kA, oe, jx, eX, aA, cz, AZ, j, v, bX, Qo, V, q, J, eA, UE, yw, uT, aT, yu, dz, jj, an, bau, ae, af, eu, kl, alX, s, aDf, atl, r, lo, rs, eS, sK, xw, el, aDQ, ck, BU, rR, ac, c, VJ, wg, aUW, bbA, hr, mf, cS, aDR, gT, bt, ao, vi, Kd, am, qX, qb, uG, jh, aHv, uu, jE, fu, eq, hF, ad, dZ, kL, qC, fU, hX, wD, F, oz, sO, tT, qh, oW, bHQ, mu, uX, VX, BO, et, cH, Fm, bHX, eZ, yy, bHV, aF, hp, S, aot, iR, dr, ek, D, gk, Bv, qe, pc, bE, ag, QV, ll, gZ, cP, ei, hE, aed, alq, y); #c1(HFE) c2(NP_620577) c3(hereditary hemochromatosis protein isoform 8 precursor) c4(237750) c5(237751, 237752, 237753, 237754, 237755, 237756) c6( ) c7(oO, atT, T, fR, ea, if, e, al, fd, bB, cL, Vd, ci, bHU, aY, ip, x, aEQ, bHS, dY, aE, rw, Gb, M, dV, K, bHT, wt, dQ, qD, dF, N, aiJ, tw, aa, hm, zQ, pr, aW, adw, az, bGL, hl, LZ, fc, bHW, alb, g, as, cm, bw, bHP, wT, bwQ, eY, aBr, rq, aNv, iT, aGe, mx, U, cj, vy, Gs, cx, dW, QF, k, m, abX, pw, dD, gU, aw, ht, oY, aj, fr, aUX, bHO, cd, br, ai, bbB, cc, hw, pN, Bj, aMD, h, eV, dL, ZN, A, sq, aq, pm, mo, bHR, Zj, Wt, op, vU, GG, cO, mg, at, eo, aDE, B, bL, bGu, jv, pF, eR, kA, oe, jx, eX, aA, cz, QZ, j, v, bX, Qo, V, q, J, eA, UE, yw, uT, aT, yu, dz, jj, an, bau, ae, af, eu, kl, alX, s, aDf, atl, r, lo, rs, eS, sK, xw, el, aDQ, ck, BU, rR, ac, c, VJ, wg, aUW, bbA, hr, mf, cS, aDR, gT, bt, ao, vi, Kd, am, qX, qb, uG, jh, aHv, uu, jE, fu, eq, hF, ad, dZ, kL, qC, fU, hX, wD, F, oz, sO, tT, qh, oW, bHQ, mu, uX, VX, BO, et, cH, Fm, bHX, eZ, yy, bHV, aF, hp, S, aot, iR, dr, ek, D, gk, Bv, qe, pc, bE, ag, QV, ll, gZ, cP, ei, hE, aed, alq, y); #c1(HFE) c2(NP_620578) c3(hereditary hemochromatosis protein isoform 9 precursor) c4(237757) c5(237758, 237759, 237760, 237761, 237762, 237763, 237764, 237765, 237766, 237767, 237768) c6( ) c7(oO, atT, T, fR, ea, if, e, al, fd, bB, cL, Vd, ci, bHU, aY, ip, x, aEQ, bHS, dY, aE, rw, Gb, M, dV, K, bHT, wt, dQ, qD, dF, N, aiJ, tw, aa, hm, zQ, pr, aW, adw, az, bGL, hl, LZ, fc, bHW, alb, g, as, cm, bw, bHP, wT, bwQ, eY, aBr, rq, aNv, iT, aGe, mx, U, cj, vy, Gs, cX, dW, QF, k, m, abX, pw, dD, gU, aw, ht, oY, aj, fr, aUX, bHO, cd, br, ai, bbB, cc, hw, pN, Bj, aMD, h, eV, dL, ZN, A, sq, aq, pm, mo, bHR, Zj, Wt, op, vU, GG, cO, mg, at, eo, aDE, B, bL, bGu, jv, pF, eR, kA, oe, jx, eX, aA, cz, AZ, j, v, bX, Qo, V, q, J, eA, UE, yw, uT, aT, yu, dz, jj, an, bau, ae, af, eu, kl, alX, s, aDf, atl, r, lo, rs, eS, sK, xw, el, aDQ, ck, BU, rR, ac, c, VJ, wg, aUW, bbA, hr, mf, cS, aDR, gT, bt, ao, vi, Kd, am, qX, qb, uG, jh, aHv, uu, jE, fu, eq, hF, ad, dZ, kL, qC, fU, hX, wD, F, oz, sO, tT, qh, oW, bHQ, mu, uX, VX, BO, et, cH, Fm, bHX, eZ, yy, bHV, aF, hp, S, aot, iR, dr, ek, D, gk, Bv, qe, pc, bE, ag, QV, ll, gZ, cP, ei, hE, aed, alq, y); #c1(HFE) c2(NP_620579) c3(hereditary hemochromatosis protein isoform 10 precursor) c4(237769) c5(237770, 237771, 237772, 237773, 237774, 237775) c6( ) c7(oO, atT, T, fR, ea, if, e, al, fd, bB, cL, Vd, ci, bHU, aY, ip, x, aEQ, bHS, dY, aE, rw, Gb, M, dV, K, bHT, wt, dQ, qD, dF, N, aiJ, tw, aa, hm, zQ, pr, aW, adw, az, bGL, hl, LZ, fc, bHW, alb, g, as, cm, bw, bHP, wT, bwQ, eY, aBr, rq, aNv, iT, aGe, mx, U, cj, vy, Gs, cx, dW, QF, k, m, abX, pw, dD, gU, aw, ht, oY, aj, fr, aUX, bHO, cd, br, ai, bbB, cc, hw, pN, Bj, aMD, h, eV, dL, ZN, A, sq, aq, pm, mo, bHR, Zj, Wt, op, vU, GG, cO, mg, at, eo, aDE, B, bL, bGu, jv, pF, eR, kA, oe, jx, eX, aA, cz, AZ, j, v, bX, Qo, V, q, J, eA, UE, yw, uT, aT, yu, dz, jj, an, bau, ae, af, eu, kl, alX, s, aDf, atl, r, lo, rs, eS, sK, xw, el, aDQ, ck, BU, rR, ac, c, VJ, wg, aUW, bbA, hr, mf, cS, aDR, gT, bt, ao, vi, Kd, am, qX, qb, uG, jh, aHv, uu, jE, fu, eq, hF, ad, dZ, kL, qC, fU, hX, wD, F, oz, sO, tT, qh, oW, bHQ, mu, uX, VX, BO, et, cH, Fm, bHX, eZ, yy, bHV, aF, hp, S, aot, iR, dr, ek, D, gk, Bv, qe, pc, bE, ag, QV, ll, gZ, cP, ei, hE, aed, alq, y); #c1(HFE) c2(NP_620580) c3(hereditary hemochromatosis protein isoform Il precursor) c4(237776) c5(237777, 237778, 237779, 237780, 237781, 237782) c6( ) c7(oO, atT, T, fR, ea, if, e, al, fd, bB, cL, Vd, ci, bHU, aY, ip, x, aEQ, bHS, dY, aE, rw, Gb, M, dV, K, bHT, wt, dQ, qD, dF, N, aiJ, tw, aa, hm, zQ, pr, aW, adw, az, bGL, hl, LZ, fc, bHW, alb, g, as, cm, bw, bHP, wT, bwQ, eY, aBr, rq, aNv, iT, aGe, mx, U, cj, vy, Gs, cX, dW, QF, k, m, abX, pw, dD, gU, aw, ht, oY, aj, fr, aUX, bHO, cd, br, ai, bbB, cc, hw, pN, Bj, aMD, h, eV, dL, ZN, A, sq, aq, pm, mo, bHR, Zj, Wt, op, vU, GG, cO, mg, at, eo, aDE, B, bL, bGu, jv, pF, eR, kA, oe, jx, eX, aA, cz, AZ, j, v, bX, Qo, V, q, J, eA, UE, yw, uT, aT, yu, dz, jj, an, bau, ae, af, eu, kl, alX, s, aDf, atl, r, lo, rs, eS, sK, xw, el, aDQ, ck, BU, rR, ac, c, VJ, wg, aUW, bbA, hr, mf, cS, aDR, gT, bt, ao, vi, Kd, am, qX, qb, uG, jh, alv, uu, jE, fu, eq, hF, ad, dZ, kL, qC, fU, hX, wD, F, oz, sO, tT, qh, oW, bHQ, mu, uX, VX, BO, et, cH, Fm, bHX, eZ, yy, bHV, aF, hp, S, aot, iR, dr, ek, D, gk, Bv, qe, pc, bE, ag, QV, ll, gZ, cP, ei, hE, aed, alq, y); #c1(HFE) c2(XP_011512846) c3(hereditary hemochromatosis protein isoform X2) c4(303921) c5(303922, 303923, 303924, 303925, 303926, 303927) c6( ) c7(oO, atT, T, fR, ea, if, e, al, fd, bB, cL, Vd, ci, bHU, aY, ip, x, aEQ, bHS, dY, aE, rw, Gb, M, dV, K, bHT, wt, dQ, qD, dF, N, aiJ, tw, aa, hm, zQ, pr, aW, adw, az, bGL, hl, LZ, fc, bHW, alb, g, as, cm, bw, bHP, wT, bwQ, eY, aBr, rq, aNv, iT, aGe, mx, U, cj, vy, Gs, cX, dW, QF, k, m, abX, pw, dD, gU, aw, ht, oY, aj, fr, aUX, bHO, cd, br, ai, bbB, cc, hw, pN, Bj, aMD, h, eV, dL, ZN, A, sq, aq, pm, mo, bHR, Zj, Wt, op, vU, GG, cO, mg, at, eo, aDE, B, bL, bGu, jv, pF, eR, kA, oe, jx, eX, aA, cz, AZ, j, v, bX, Qo, V, q, J, eA, UE, yw, uT, aT, yu, dz, jj, an, bau, ae, af, eu, kl, alX, s, aDf, atl, r, lo, rs, eS, sK, xw, el, aDQ, ck, BU, rR, ac, c, VJ, wg, aUW, bbA, hr, mf, cS, aDR, gT, bt, ao, vi, Kd, am, qX, qb, uG, jh, aHv, uu, jE, fu, eq, hF, ad, dZ, kL, qC, fU, hX, wD, F, oz, sO, tT, qh, oW, bHQ, mu, uX, VX, BO, et, CH, Fm, bHX, eZ, yy, bHV, aF, hp, S, aot, iR, dr, ek, D, gk, Bv, qe, pc, bE, ag, QV, ll, gZ, cP, ei, hE, aed, alq, y); #c1(HFM1) c2(XP_011539154) c3(probable ATP-dependent DNA helicase HFM1 isoform X1) c4(337241) c5(337242, 337243, 337244, 337245, 337246, 337247) c6( ) c7(bHZ, T, wr, bHY, MR, A, aW, ask, bnw, rC, CT, ew, aT, hD, lo, dy, La, pb, q, atb); #c1(HFM1) c2(XP_011539155) c3(probable ATP-dependent DNA helicase HFM1 isoform X4) c4(337248) c5( ) c6( ) c7(bHZ, T, wr, bHY, MR, A, aW, ask, bnw, rC, CT, ew, aT, hD, lo, dy, La, pb, q, atb); #c1(HFM1) c2(XP_011539156) c3(probable ATP-dependent DNA helicase HFM1 isoform X5) c4(337249) c5(337250) c6( ) c7(bHZ, T, wr, bHY, MR, A, aW, ask, bnw, rC, CT, ew, al, hD, lo, dy, La, pb, q, atb); #c1(HFM1) c2(XP_011539157) c3(probable ATP-dependent DNA helicase HFM1 isoform X6) c4(337251) c5( ) c6( ) c7(bHZ, T, wr, bHY, MR, A, aW, ask, bnw, rC, CT, ew, aT, HD, lo, dy, La, pb, q, atb); #c1(HFM1) c2(XP_011539158) c3(probable ATP-dependent DNA helicase HFM1 isoform X7) c4(337252) c5(337253, 337254, 337255, 337256, 337257) c6( ) c7(bHZ, T, wr, bHY, MR, A, aW, ask, bnw, rC, CT, ew, aT, hD, lo, dy, La, pb, q, atb); #c1(HFM1) c2(XP_011539159) c3(probable ATP-dependent DNA helicase HFM1 isoform X8) c4(337258) c5( ) c6( ) c7(bHZ, T, wr, bHY, MR, A, aW, ask, bnw, rC, CT, ew, aT, HD, lo, dy, La, pb, q, atb); #c1(HFM1) c2(XP_011539162) c3(probable ATP-dependent DNA helicase HFM1 isoform X1I) c4( ) c5(337259, 337260, 337261, 337262, 337263, 337264, 337265, 337266, 337267, 337268, 337269, 337270) c6( ) c7(bHZ, T, wr, bHY, MR, A, aW, ask, bnw, rC, CT, ew, aT, HD, lo, dy, La, pb, q, atb); #c1(HGD) c2(NP_000178) c3(homogentisate 1,2-dioxygenase) c4(3868) c5(3869, 3870, 3871, 3872, 3873, 3874) c6( ) c7(blc, blb, mO, jr, s, bld, bla, dq, iV, aY, ai, Us); #c1(HGF) c2(NP_000592) c3(hepatocyte growth factor isoform I preproprotein) c4(13398, 13399) c5(13400, 13401, 13402, 13403, 13404, 13405, 13406, 13407, 13408, 13409, 13410, 13411, 13412, 13413, 13414, 13415, 13416, 13417, 13418, 13419, 13420, 13421, 13422, 13423, 13424, 13425, 13426, 13427, 13428, 13429, 13430, 13431, 13432, 13433, 13434, 13435, 13436, 13437, 13438, 13439, 13440, 13441, 13442, 13443, 13444, 13445, 13446, 13447, 13448, 13449, 13450, 13451, 13452, 13453, 13454, 13455, 13456, 13457, 13458, 13459, 13460, 13461, 13462, 13463, 13464, 13465, 13466, 13467, 13468, 13469, 13470, 13471, 13472, 13473, 13474, 13475, 13476, 13477, 13478, 13479, 13480, 13481, 13482, 13483, 13484, 13485, 13486, 13487, 13488, 13489, 13490, 13491, 13492, 13493) c6(13494) c7(oO, T, asg, aC, pf, ea, if, e, al, bll, fd, bB, axk, Vd, ci, qj, lR, sD, blk, blg, bv, dY, aE, fm, aeO, ja, Vv, Ou, HR, K, hu, ex, eL, bc, cp, aa, hm, DA, eG, aeR, iE, HI, LC, EV, bvR, az, aW, hl, LZ, fc, CT, bjk, brr, g, as, eF, cm, Qt, iP, qJ, blj, eM, nB, iA, acS, acw, mx, U, vy, wB, wx, Hb, m, jM, fy, dD, zt, aw, ht, NT, RC, aBx, n, cQ, aj, fr, bd, qN, cd, asS, ai, ft, jz, cc, zw, Nn, gd, KH, lG, GT, dL, bl, eV, hB, vx, Ha, aqi, A, blh, fQ, aq, fe, kp, yq, pm, boD, ar, kT, Sc, iO, asl, dB, hk, PI, lf, Be, hL, at, bjk, uz, fL, bli, B, cn, bL, jv, fM, dA, AS, jp, eR, fG, oe, eX, aA, cR, cz, fF, v, j, bX, q, J, er, eA, asG, Yn, uT, blf, aT, cy, jj, bq, an, rz, bau, ae, dl, bA, eu, jf, Uf, Sh, s, nO, iU, oV, Y, ev, bg, r, aYp, lo, en, aDY, cl, t, iK, pb, ck, ov, sS, rR, ac, c, wg, nM, wl, hc, iM, QG, agf, cS, dy, fD, Bd, bt, lJ, ao, vi, hS, On, hl, kh, tS, aD, iW, jh, vr, dG, eq, fu, iJ, mC, ad, qC, fU, dP, hd, ib, MS, av, Db, yt, sN, py, lC, LA, rX, ab, io, hP, ble, ji, uQ, d, lR, je, cW, qt, vj, ff, eH, eZ, aJW, aF, Sm, iR, iV, KM, bz, dr, qa, eK, LB, es, gk, Bv, Ew, KD, bE, AR, aVv, afb, ll, wz, gZ, Hs, aS, cP, ei, GF, hE, vt, iu, y, pC); #c1(HGF) c2(NP_001010931) c3(hepatocyte growth factor isoform 2 precursor) c4(24192) c5(24193, 24194, 24195, 24196, 24197, 24198, 24199, 24200, 24201, 24202, 24203, 24204, 24205) c6( ) c7(oO, T, asg, aC, pf, ea, if, e, al, bll, fd, bB, axk, Vd, ci, qj, lR, sD, blk, blg, bv, dY, aE, fm, aeO, ja, Vv, Ou, HR, K, hu, ex, eL, bc, cp, aa, hm, DA, eG, aeR, iE, HI, LC, EV, bvR, az, aW, hl, LZ, fc, CT, bjk, brr, g, as, eF, cm, At, iP, qJ, blj, eM, nB, iA, acS, acw, mx, U, vy, wB, wx, Hb, m, jM, fy, dD, zt, aw, ht, NT, RC, aBx, n, cQ, aj, fr, bd, qN, cd, asS, ai, ft, jz, cc, zw, Nn, gd, KH, lG, GT, dL, bl, eV, hB, vx, Ha, aqi, A, blh, fl, aq, fe, kp, yq, pm, boD, ar, KT, Sc, iO, asl, dB, hk, PI, lf, Be, hL, at, bjk, uz, fL, bli, B, cn, bL, jv, fM, dA, AS, jp, eR, fG, oe, eX, aA, cR, cz, fF, v, j, bX, q, J, er, eA, asG, Yn, uT, blf, aT, cy, jj, bq, an, rz, bau, ae, dl, bA, eu, jf, Uf, Sh, s, nO, il, oV, Y, ev, bg, r, aYp, lo, en, aDY, cl, t, iK, pb, ck, ov, sS, rR, ac, c, wg, nM, wl, hc, iM, QG, agf, cS, dy, fD, Bd, bt, lJ, ao, vi, hS, On, hl, kh, tS, aD, iW, jh, vr, dG, eq, fu, iJ, mC, ad, qC, fU, dP, hd, ib, MS, av, Db, yt, sN, py, lC, LA, rX, ab, io, hP, ble, ji, ul, d, lR, je, cW, qt, vj, ff, eH, eZ, aJW, aF, Sm, iR, iV, KM, bz, dr, qa, eK, LB, es, gk, Bv, Ew, KD, bE, AR, aVv, aFb, ll, wz, gZ, Hs, aS, cP, ei, GF, hE, vt, iu, y, pC); #c1(HGF) c2(NP_001010932) c3(hepatocyte growth factor isoform 3 precursor) c4(24206, 24207, 24208) c5(24209, 24210, 24211, 24212, 24213, 24214, 24215) c6( ) c7(oO, T, asg, aC, pf, ea, if, e, al, bll, fd, bB, axk, Vd, ci, qj, lR, sD, blk, blg, bv, dY, aE, fm, aeO, ja, Vv, Ou, HR, K, hu, ex, eL, bc, cp, aa, hm, DA, eG, aeR, iE, HI, LC, EV, bvR, az, aW, hl, LZ, fc, CT, bjk, brr, g, as, eF, cm, Qt, iP, qJ, blj, eM, nB, iA, acS, acw, mx, U, vy, wB, wx, Hb, m, jM, fy, dD, zt, aw, ht, NT, RC, aBx, n, cQ, aj, fr, bd, qN, cd, asS, ai, ft, jz, cc, zw, Nn, gd, KH, lG, GT, dL, bl, eV, hB, vx, Ha, aqi, A, blh, fQ, aq, fe, kp, yq, pm, boD, ar, kT, Sc, iO, asl, dB, hk, PI, lf, Be, hL, at, bjK, uz, fL, bli, B, cn, bL, jv, fM, dA, AS, jp, eR, fG, oe, eX, aA, cR, cz, fF, v, j, bX, q, J, er, eA, asG, Yn, uT, blf, aT, cy, jj, bq, an, rz, bau, ae, dl, bA, eu, jf, Uf, Sh, s, nO, iU, oV, Y, ev, bg, r, aYp, lo, en, aDY, cl, t, iK, pb, ck, ov, sS, rR, ac, c, wg, nM, wl, hc, iM, QG, agf, cS, dy, fD, Bd, bt, lJ, ao, vi, hS, On, hl, kh, tS, aD, iW, jh, vr, dG, eq, fu, iJ, mC, ad, qC, fU, dP, hd, ib, MS, av, Db, yt, sN, py, lC, LA, rX, ab, io, hP, ble, ji, uQ, d, lR, je, cW, qt, vj, ff, eH, eZ, aJW, aF, Sm, iR, iV, KM, bz, dr, qa, eK, LB, es, gk, Bv, Ew, KD, bE, AR, aVv, aFb, ll, wz, gZ, Hs, aS, cP, ei, GF, hE, vt, iu, y, pC); #c1(HGF) c2(NP_001010933) c3(hepatocyte growth factor isoform 4 precursor) c4(24216) c5(24217, 24218, 24219, 24220, 24221, 24222, 24223) c6( ) c7(oO, T, asg, aC, pf, ea, if, e, al, bll, fd, bB, axk, Vd, ci, qj, lR, sD, blk, blg, bv, dY, aE, fm, aeO, ja, Vv, Ou, HR, K, hu, ex, eL, bc, cp, aa, hm, DA, eG, aeR, iE, HI, LC, EV, bvR, az, aW, hl, LZ, fc, CT, bjk, brr, g, as, eF, cm, At, iP, qJ, blj, eM, nB, iA, acS, acw, mx, U, vy, wB, wx, Hb, m, jM, fy, dD, zt, aw, ht, NT, RC, aBx, n, cQ, aj, fr, bd, qN, cd, asS, ai, ft, jz, cc, zw, Nn, gd, KH, lG, GT, dL, bl, eV, hB, vx, Ha, aqi, A, blh, fQ, aq, fe, kp, yq, pm, boD, ar, kT, Sc, iO, asl, dB, hk, PI, lf, Be, hl, at, bjk, uz, fL, bli, B, cn, bL, jv, fM, dA, AS, jp, eR, fG, oe, eX, aA, cR, cz, fF, v, j, bX, q, J, er, eA, asG, Yn, uT, blf, aT, cy, jj, bq, an, rz, bau, ae, dl, bA, eu, jf, Uf, Sh, s, nO, iU, oV, Y, ev, bg, r, aYp, lo, en, aDY, cl, t, iK, pb, ck, ov, sS, rR, ac, c, wg, nM, wl, hc, iM, QG, agf, cS, dy, fD, Bd, bt, lJ, ao, vi, hS, On, hl, kh, tS, aD, iW, jh, vr, dG, eq, fu, iJ, mC, ad, qC, fU, dP, hd, ib, MS, av, Db, yt, sN, py, lC, LA, rX, ab, io, hP, ble, ji, ul, d, lR, je, cW, qt, vj, ff, eH, eZ, aJW, aF, Sm, iR, iV, KM, bz, dr, qa, eK, LB, es, gk, Bv, Ew, KD, bE, AR, aVv, aFb, ll, wz, gZ, Hs, aS, cP, ei, GF, hE, vt, iu, y, pC); #c1(HGF) c2(NP_001010934) c3(hepatocyte growth factor isoform 5 precursor) c4(24224, 24225, 24226, 24227) c5(24228, 24229, 24230, 24231, 24232, 24233, 24234, 24235, 24236, 24237, 24238, 24239, 24240, 24241, 24242, 24243, 24244, 24245, 24246, 24247, 24248, 24249, 24250, 24251, 24252) c6( ) c7(oO, T, asg, aC, pf, ea, if, e, al, bll, fd, bB, axk, Vd, ci, qj, lR, sD, blk, blg, bv, dY, aE, fm, aeO, ja, Vv, Ou, HR, K, hu, ex, eL, bc, cp, aa, hm, DA, eG, aeR, iE, HI, LC, EV, bvR, az, aW, hl, LZ, fc, CT, bjk, brr, g, as, eF, cm, At, iP, qJ, blj, eM, nB, iA, acS, acw, mx, U, vy, wB, wx, Hb, m, jM, fy, dD, zt, aw, ht, NT, RC, aBx, n, cQ, aj, fr, bd, qN, cd, asS, ai, ft, jz, cc, zw, Nn, gd, KH, lG, GT, dL, bl, eV, hB, vx, Ha, aqi, A, blh, fQ, aq, fe, kp, yq, pm, boD, ar, kT, Sc, iO, asl, dB, hk, PI, lf, Be, hL, at, bjk, uz, fL, bli, B, cn, bL, jv, fM, dA, AS, jp, eR, fG, oe, eX, aA, cR, cz, fF, v, j, bX, q, J, er, eA, asG, Yn, uT, blf, aT, cy, jj, bq, an, rz, bau, ae, dl, bA, eu, jf, Uf, Sh, s, no, iU, oV, Y, ev, bg, r, aYp, lo, en, aDY, cl, t, iK, pb, ck, ov, sS, rR, ac, c, wg, nM, wO, hc, iM, QG, agf, cS, dy, fD, Bd, bt, lJ, ao, vi, hS, On, hl, kh, tS, aD, iW, jh, vr, dG, eq, fu, iJ, mC, ad, qC, fU, dP, hd, ib, MS, av, Db, yt, sN, py, lC, LA, rX, ab, io, hP, ble, ji, ul, d, lR, je, cW, qt, vj, ff, eH, eZ, aJW, aF, Sm, iR, iV, KM, bz, dr, qa, eK, LB, es, gk, Bv, Ew, KD, bE, AR, aVv, aFb, ll, wz, gZ, Hs, aS, cP, ei, GF, hE, vt, iu, y, pC); #c1(HGFAC) c2(NP_001519) c3(hepatocyte growth factor activator isoform 2 preproprotein) c4(118467) c5(118468, 118469, 118470, 118471, 118472, 118473) c6( ) c7(hl, aC, d, fM, aE, amg, vj, if, hL, fd, fr, ci, m, Fq, qN, t, g, ae, B, ad, cl); #c1(HGH1) c2(NP_057542) c3(protein HGH1 homolog) c4(191314, 191315) c5(191316, 191317, 191318, 191319, 191320, 191321, 191322, 191323, 191324, 191325, 191326, 191327) c6( ) c7(n, bo, Rj); #c1(HGS) c2(NP_004703) c3(hepatocyte growth factor-regulated tyrosine kinase substrate) c4(148939, 148940) c5(148941, 148942, 148943, 148944, 148945, 148946) c6( ) c7(T, dD, uk, ea, ex, cz, n, ajM, bS, eq, ct, MF, q, aR, h, eV, HO, s, Ls, lb, r, Z, cm, Qt, Ew, W, bE); #c1(HGS) c2(XP_011523765) c3(hepatocyte growth factor-regulated tyrosine kinase substrate isoform X1) c4( ) c5(318259, 318260, 318261, 318262, 318263, 318264) c6( ) c7(T, dD, uk, ea, ex, cz, n, ajM, bS, eq, ct, MF, q, aR, h, eV, HO, s, Ls, lb, r, Z, cm, Qt, Ew, W, bE); #c1(HGSNAT) c2(NP_689632) c3(heparan-alpha-glucosaminide N-acetyltransferase precursor) c4(243354, 243355, 243356, 243357) c5(243358, 243359, 243360, 243361, 243362, 243363, 243364, 243365, 243366, 243367, 243368, 243369, 243370, 243371, 243372, 243373, 243374, 243375) c6( ) c7(agG, aHX, U, kg); #c1(HGSNAT) c2(XP_005273469) c3(heparan-alpha-glucosaminide N-acetyltransferase isoform X4) c4( ) c5(284599, 284600, 284601, 284602, 284603, 284604) c6( ) c7(agG, aHX, U, kg); #c1(HHAT) c2(NP_001164035) c3(protein-cysteine N-palmitoyltransferase HHAT isoform 2) c4(66524) c5(66525, 66526, 66527, 66528, 66529, 66530, 66531, 66532, 66533, 66534, 66535, 66536) c6( ) c7(T, tQ, Bm, BF, q, ad, eA, hW, brH, sO, h, Nc, K, ae, bin, blm, aOX, d, bf, oV, Y, r, kp, cm, apM, brG, Qu, hc, ag, cj, bt); #c1(HHAT) c2(NP_001164051) c3(protein-cysteine N-palmitoyltransferase HHAT isoform 1) c4(66537, 66538) c5(66539, 66540, 66541, 66542, 66543, 66544, 66545, 66546, 66547, 66548, 66549, 66550, 66551, 66552, 66553, 66554, 66555) c6( ) c7(T, tQ, Bm, BF, q, ad, eA, hW, brH, sO, h, Nc, K, ae, bln, blm, aDX, d, bf, oV, Y, r, kp, cm, apM, brG, Qu, hc, ag, cj, bt); #c1(HHAT) c2(NP_001164058) c3(protein-cysteine N-palmitoyltransferase HHAT isoform 4) c4(66563) c5(66564, 66565, 66566, 66567, 66568, 66569, 66570) c6( ) c7(T, tQ, Bm, BF, q, ad, eA, hW, brH, sO, h, Nc, K, ae, bin, blm, aOX, d, bf, oV, Y, r, kp, cm, apM, brG, Qu, hc, ag, cj, bt); #c1(HHAT) c2(NP_001164059) c3(protein-cysteine N-palmitoyltransferase HHAT isoform 3) c4(66571, 66572) c5(66573, 66574, 66575, 66576, 66577, 66578, 66579, 66580, 66581, 66582, 66583) c6( ) c7(T, tQ, Bm, BF, q, ad, eA, hW, brH, sD, h, Nc, K, ae, bln, blm, alX, d, bf, oV, Y, r, kp, cm, apM, brG, Qu, hc, ag, cj, bt); #c1(HHAT) c2(XP_006711504) c3(protein-cysteine N-palmitoyltransferase HHAT isoform X2) c4(286042) c5(286043, 286044, 286045) c6( ) c7(T, tQ, Bm, BF, q, ad, eA, hW, brH, sO, h, Nc, K, ae, bln, blm, aDX, d, bf, oV, Y, r, kp, cm, apM, brG, Qu, hc, ag, cj, bt); #c1(HHAT) c2(XP_011508039) c3(protein-cysteine N-palmitoyltransferase HHAT isoform X1) c4( ) c5(298511) c6( ) c7(T, tQ, Bm, BF, q, ad, eA, hW, brH, sO, h, Nc, K, ae, bin, blm, aOX, d, bf, oV, Y, r, kp, cm, apM, brG, Qu, hc, ag, cj, bt); #c1(HHAT) c2(XP_011508043) c3(protein-cysteine N-palmitoyltransferase HHAT isoform X4) c4(298512) c5(298513, 298514, 298515, 298516, 298517) c6( ) c7(T, tQ, Bm, BF, q, ad, eA, hW, brH, sO, h, Nc, K, ae, bln, blm, aOX, d, bf, oV, Y, r, kp, cm, apM, brG, Qu, hc, ag, cj, bt); #c1(HHAT) c2(XP_011508047) c3(protein-cysteine N-palmitoyltransferase HHAT isoform X8) c4( ) c5(298518) c6( ) c7(T, tQ, Bm, BF, q, ad, eA, hW, brH, sO, h, Nc, K, ae, bln, blm, aOX, d, bf, oV, Y, r, kp, cm, apM, brG, Qu, hc, ag, cj, bt); #c1(HHAT) c2(XP_011508048) c3(protein-cysteine N-palmitoyltransferase HHAT isoform X9) c4(298519) c5( ) c6( ) c7(T, tQ, Bm, BF, q, ad, eA, hW, brH, sO, h, Nc, K, ae, bln, blm, aOX, d, bf, oV, Y, r, kp, cm, apM, brG, Qu, hc, ag, cj, bt); #c1(HHAT) c2(XP_011508049) c3(protein-cysteine N-palmitoyltransferase HHAT isoform X10) c4( ) c5(298520, 298521, 298522, 298523, 298524) c6( ) c7(T, tQ, Bm, BF, q, ad, eA, hW, brH, sO, h, Nc, K, ae, bln, blm, aDX, d, bf, oV, Y, r, kp, cm, apM, brG, Qu, hc, ag, cj, bt); #c1(HHATL) c2(NP_065758) c3(protein-cysteine N-palmitoyltransferase HHAT-like protein) c4( ) c5(205217, 205218, 205219, 205220, 205221, 205222, 205223, 205224, 205225, 205226, 205227, 205228, 205229, 205230, 205231, 205232, 205233, 205234) c6( ) c7(K); #c1(HHATL) c2(XP_011532271) c3(protein-cysteine N-palmitoyltransferase HHAT-like protein isoform X2) c4(328329, 328330, 328331) c5(328332, 328333, 328334, 328335) c6( ) c7(K); #c1(HHEX) c2(NP_002720) c3(hematopoietically-expressed homeobox protein HHEX) c4(131399) c5(131400, 131401, 131402, 131403, 131404, 131405, 131406) c6( ) c7(bL, dD, aZ, bDa, aj, dG, j, ci, v, eq, V, fK, J, aT, h, af, aQy, kl, A, aW, fc, zR, mo, eU, nB, mg, hL, awM, mf, lS, ei, bt, AN); #c1(HHIP) c2(NP_071920) c3(hedgehog-interacting protein precursor) c4(212309, 212310) c5(212311, 212312, 212313, 212314, 212315, 212316) c6( ) c7(bL, T, EM, aC, al, aA, bB, aj, v, ci, bh, q, J, ad, eA, aE, K, aa, jf, avP, aq, kp, aF, g, iV, cm, fq, bz, iO, gk, bE, hk, at, Dp, xb, B); #c1(HHIP) c2(XP_006714351) c3(hedgehog-interacting protein isoform X2) c4( ) c5(287781, 287782, 287783, 287784, 287785, 287786) c6( ) c7(bL, T, EM, aC, al, aA, bB, aj, v, ci, bh, q, J, ad, eA, aE, K, aa, jf, avP, aq, kp, aF, g, iV, cm, fq, bz, iO, gk, bE, hk, at, Dp, xb, B); #c1(HHIPL1) c2(NP_001120730) c3(HHIP-like protein 1 isoform a precursor) c4(44749) c5(44750, 44751, 44752, 44753, 44754, 44755, 44756, 44757) c6(44758, 44759) c7(j); #c1(HHIPL1) c2(NP_115801) c3(HHIP-like protein 1 isoform b precursor) c4(223950) c5(223951, 223952, 223953, 223954, 223955, 223956, 223957, 223958) c6(223959, 223960) c7(j); #c1(HHLA1) c2(NP_001138567) c3(HERV-H LTR-associating protein 1 precursor) c4(56997) c5(56998, 56999, 57000, 57001, 57002, 57003) c6( ) c7(Hn); #c1(HHLA2) c2(NP_001269487) c3(HERV-H LTR-associating protein 2 isoform b precursor) c4(101740) c5( ) c6( ) c7(ac); #c1(HHLA2) c2(NP_009003) c3(HERV-H LTR-associating protein 2 isoform a precursor) c4(170181, 170182, 170183) c5(170184, 170185, 170186, 170187, 170188, 170189, 170190, 170191, 170192, 170193, 170194, 170195, 170196, 170197) c6( ) c7(ac); #c1(HHLA2) c2(XP_011510669) c3(HERV-H LTR-associating protein 2 isoform X3) c4( ) c5(301114, 301115, 301116, 301117, 301118, 301119, 301120, 301121, 301122, 301123, 301124, 301125, 301126, 301127, 301128, 301129, 301130, 301131) c6( ) c7(ac); #c1(HIAT1) c2(XP_011540259) c3(hippocampus abundant transcript 1 protein isoform X2) c4(338674) c5( ) c6( ) c7(af); #c1(HIAT1) c2(XP_011540260) c3(hippocampus abundant transcript 1 protein isoform X3) c4( ) c5(338675, 338676, 338677, 338678, 338679, 338680) c6( ) c7(af); #c1(HIBADH) c2(NP_689953) c3(3-hydroxyisobutyrate dehydrogenase, mitochondrial precursor) c4(244506) c5(244507, 244508, 244509, 244510, 244511, 244512) c6( ) c7(cd, dy, SB); #c1(HIBCH) c2(NP_055177) c3(3-hydroxyisobutyryl-CoA hydrolase, mitochondrial isoform 1 precursor) c4(179585, 179586) c5(179587, 179588, 179589, 179590, 179591, 179592) c6( ) c7(blo, fq, ajU); #c1(HIBCH) c2(NP_932164) c3(3-hydroxyisobutyryl-CoA hydrolase, mitochondrial isoform 2 precursor) c4(258849) c5(258850, 258851, 258852, 258853, 258854, 258855) c6( ) c7(blo, fq, ajU); #c1(HIBCH) c2(XP_011509256) c3(3-hydroxyisobutyryl-CoA hydrolase, mitochondrial isoform X2) c4( ) c5(299892, 299893, 299894, 299895, 299896, 299897) c6( ) c7(blo, fq, ajU); #c1(HIC1) c2(NP_001091672) c3(hypermethylated in cancer 1 protein isoform 2) c4(38635, 38636) c5(38637, 38638, 38639, 38640, 38641, 38642, 38643) c6( ) c7(T, os, fM, Tb, if, lH, qx, al, aw, aA, bB, n, oZ, fd, fr, ci, qN, jh, AD, q, ad, eA, eJ, iC, bv, iu, Tg, Na, aE, blp, TK, aT, Nc, K, Dz, jj, ae, pl, jf, ZH, s, CN, qt, aW, iU, ff, az, r, fe, aF, en, hf, eF, t, cm, iK, Ld, iV, ck, gk, YE, kP, Qu, gX, m, bt, kS, B, aAO, vi); #c1(HIC1) c2(NP_006488) c3(hypermethylated in cancer 1 protein isoform 1) c4( ) c5(165632, 165633, 165634, 165635, 165636) c6( ) c7(T, os, fM, Tb, if, lH, qx, al, aw, aA, bB, n, oZ, fd, fr, ci, qN, jh, AD, q, ad, eA, eJ, iC, bv, iu, Tg, Na, aE, blp, TK, aT, Nc, K, Dz, jj, ae, pl, jf, ZH, s, CN, qt, aW, il, ff, az, r, fe, aF, en, hf, eF, t, cm, iK, Ld, iV, ck, gk, YE, kP, Qu, gX, m, bt, kS, B, aAO, vi); #c1(HIC2) c2(NP_055909) c3(hypermethylated in cancer 2 protein) c4(184444) c5(184445, 184446, 184447, 184448, 184449, 184450, 184451, 184452, 184453, 184454, 184455, 184456, 184457, 184458) c6( ) c7(md, agY, eZ); #c1(HID1) c2(NP_085133) c3(protein HID1) c4(219374, 219375) c5(219376, 219377, 219378, 219379, 219380, 219381, 219382, 219383, 219384, 219385, 219386, 219387) c6( ) c7(il, aW, T, zZ, aT); #c1(HIF1A) c2(NP_001230013) c3(hypoxia-inducible factor 1-alpha isoform 3) c4(85787, 85788, 85789) c5(85790, 85791, 85792, 85793, 85794, 85795, 85796) c6( ) c7(oO, gh, T, bM, lD, aC, fR, auW, ea, if, e, al, bll, eb, bB, fd, cL, ci, add, Hr, ip, wa, aJO, aE, Bc, fm, bAy, aed, HR, K, qM, hu, aCU, eL, bc, aiJ, cp, De, aa, ly, DA, hm, eG, yo, JI, LC, aAg, blt, aW, XV, LZ, fc, hN, vt, vs, zC, g, as, eF, cm, uC, iP, aik, y, lt, eM, nB, iA, go, vA, U, tV, cj, vy, tG, nw, m, ym, fy, ik, iu, dD, sw, zt, vX, ht, NT, qy, wh, aw, n, aj, fr, qN, rP, zP, cd, md, aky, BI, ft, cc, zw, Cf, gd, Lv, lG, cU, Nf, dL, jy, Jn, h, eV, pi, cY, wC, hi, avc, bFc, hQ, fe, aq, kp, yq, mo, YF, avf, op, lx, Ye, W, bP, dB, hk, bsO, bo, ayS, lf, hL, JM, hR, E, at, qF, anx, it, B, bkV, bL, jv, wj, dA, eR, mr, fG, oe, lH, aGu, eX, aA, buk, cz, hy, v, j, rf, C, AE, lu, uW, V, AO, q, J, o, eA, azn, eJ, blr, oT, XX, RI, yw, Mg, rS, yg, al, cy, rO, nQ, j, bq, sR, ae, dl, eu, jf, qw, s, De, NE, nO, Y, blq, fO, ev, iU, r, gm, en, afs, cl, t, apx, iK, pb, ck, nM, kP, ac, gX, fP, wR, mf, dy, fD, im, bt, rA, VB, ao, gx, Kd, kh, id, Mb, eP, MH, qb, Dd, LX, CI, rQ, iW, lD, vr, jh, aET, dG, eq, Fb, pz, ad, afb, BY, Jm, hX, av, hd, ib, py, Db, yt, sN, sO, Ot, nl, oW, hD, ji, fb, Yj, d, lR, lU, qt, ff, eH, nh, dq, aeV, Ej, VI, aDd, hp, avG, aF, iR, bz, Hv, eK, bls, Hp, es, gk, baa, pu, bF, bE, z, uR, blu, aS, GF, lC, tP, X, bEt, BA); #c1(HIF1A) c2(NP_001521) c3(hypoxia-inducible factor 1-alpha isoform 1) c4(118474, 118475) c5(118476, 118477, 118478, 118479, 118480, 118481, 118482, 118483, 118484, 118485, 118486, 118487, 118488, 118489, 118490, 118491, 118492, 118493, 118494, 118495, 118496, 118497, 118498, 118499, 118500, 118501, 118502, 118503, 118504, 118505, 118506, 118507, 118508, 118509, 118510, 118511, 118512, 118513, 118514, 118515, 118516, 118517, 118518, 118519, 118520, 118521, 118522, 118523, 118524, 118525, 118526, 118527, 118528, 118529, 118530, 118531, 118532, 118533, 118534, 118535, 118536, 118537, 118538, 118539, 118540, 118541, 118542, 118543, 118544, 118545, 118546, 118547, 118548, 118549, 118550, 118551, 118552, 118553, 118554, 118555, 118556, 118557, 118558, 118559, 118560, 118561, 118562, 118563, 118564, 118565, 118566, 118567, 118568, 118569, 118570, 118571, 118572, 118573, 118574, 118575, 118576, 118577, 118578, 118579, 118580, 118581, 118582, 118583, 118584, 118585, 118586, 118587, 118588, 118589, (18590, 118591, 118592) c6( ) c7(oO, gh, T, bM, lD, aC, fR, auW, ea, if, e, al, bll, eb, bB, fd, cL, ci, add, Hr, ip, wa, aJO, aE, Bc, fm, bAy, aed, HR, K, qM, hu, aCU, eL, bc, aiJ, cp, Qe, aa, ly, DA, hm, eG, yo, JI, LC, aAg, blt, aW, XV, LZ, fc, hN, vt, vs, zC, g, as, eF, cm, uC, iP, aik, y, lt, eM, nB, iA, go, vA, U, tV, cj, vy, tG, nw, m, ym, fy, ik, iu, dD, sw, zt, vX, ht, NT, qy, wh, aw, n, aj, fr, qN, rP, zP, cd, md, aky, BI, ft, cc, zw, Cf, gd, Lv, lG, cU, Nf, dL, jy, Jn, h, eV, pi, cY, wC, hi, avc, bFc, hQ, fe, aq, kp, yq, mo, YF, avf, op, lx, Ye, W, bP, dB, hk, bsO, bo, ayS, lf, hL, JM, hR, E, at, qF, anx, it, B, bkV, bL, jv, wj, dA, eR, mr, fG, oe, lH, aGu, eX, aA, buk, cz, hy, v, j, rf, C, AE, lu, uW, V, AD, q, J, o, eA, azn, eJ, blr, oT, XX, RI, yw, Mg, rS, yg, aT, cy, rO, nQ, j, bq, sR, ae, dl, eu, jf, qw, s, De, NE, nO, Y, blq, fO, ev, iU, r, gm, en, afs, cl, t, apx, ik, pb, ck, nM, kP, ac, gX, fP, wR, mf, dy, fD, im, bt, rA, VB, ao, gx, Kd, kh, id, Mb, eP, MH, qb, Dd, LX, CI, rQ, iW, lD, vr, jh, aET, dG, eq, Fb, pz, ad, afb, BY, Jm, hX, av, hd, ib, py, Db, yt, sN, sO, Ot, nl, oW, hD, ji, fb, Yj, d, lR, lU, qt, ff, eH, nh, dq, aeV, Ej, VI, aDd, hp, avG, aF, iR, bz, Hv, ek, bls, Hp, es, gk, baa, pu, bF, bE, z, uR, blu, aS, GF, lC, tP, X, bEt, BA); #c1(HIF1A) c2(NP_851397) c3(hypoxia-inducible factor 1-alpha isoform 2) c4(254575) c5(254576, 254577, 254578, 254579, 254580, 254581) c6( ) c7(oO, gh, T, bM, lD, aC, fR, auW, ea, if, e, al, bll, eb, bB, fd, cL, ci, add, Hr, ip, wa, aJO, aE, Bc, fm, bAy, aed, HR, K, qM, hu, aCU, eL, bc, aiJ, cp, De, aa, ly, DA, hm, eG, yo, JI, LC, aAg, blt, aW, XV, LZ, fc, hN, vt, vs, zC, g, as, eF, cm, uC, iP, aik, y, lt, eM, nB, iA, go, vA, U, tV, cj, vy, tG, nw, m, ym, fy, ik, iu, dD, sw, zt, vX, ht, NT, qy, wh, aw, n, aj, fr, qN, rP, zP, cd, md, aky, BI, ft, cc, zw, Cf, gd, Lv, lG, cU, Nf, dL, jy, Jn, h, eV, pi, cY, wC, hi, avc, bFc, hQ, fe, aq, kp, yq, mo, YF, avf, op, lx, Ye, W, bP, dB, hk, bsO, bo, ayS, lf, hL, JM, hR, E, at, qF, anx, it, B, bkV, bL, jv, wj, dA, eR, mr, fG, oe, lH, aGu, eX, aA, buk, cz, hy, v, j, rf, C, AE, lu, uW, V, AO, q, J, o, eA, azn, eJ, blr, oT, XX, RI, yw, Mg, rS, yg, al, cy, rO, nQ, j, bq, sR, ae, dl, eu, jf, qw, s, De, NE, nO, Y, blq, fO, ev, iU, r, gm, en, afs, cl, t, apx, iK, pb, ck, nM, kP, ac, gX, fP, wR, mf, dy, fD, im, bt, rA, VB, ao, gx, Kd, kh, id, Mb, eP, MH, qb, Dd, LX, CI, rQ, iW, lD, vr, jh, aET, dG, eq, Fb, pz, ad, afb, BY, Jm, hX, av, hd, ib, py, Db, yt, sN, sO, Ot, nl, oW, hD, ji, fb, Yj, d, lR, lU, qt, ff, eH, nh, dq, aeV, Ej, VI, aDd, hp, avG, aF, iR, bz, Hv, ek, bls, Hp, es, gk, baa, pu, bF, bE, z, uR, blu, aS, GF, lC, tP, X, bEt, BA); #c1(HIF1AN) c2(NP_060372) c3(hypoxia-inducible factor 1-alpha inhibitor) c4(195673, 195674) c5(195675, 195676, 195677, 195678, 195679, 195680, 195681, 195682, 195683, 195684, 195685, 195686, 195687) c6( ) c7(d, pf, at, dl, j, ci, fr, cE, af, ad); #c1(HIF1AN) c2(XP_011538242) c3(hypoxia-inducible factor 1-alpha inhibitor isoform X1) c4(335849) c5( ) c6( ) c7(d, pf, at, dL, j, ci, fr, cE, af, ad); #c1(HIF1AN) c2(XP_011538243) c3(hypoxia-inducible factor 1-alpha inhibitor isoform X2) c4( ) c5(335850, 335851, 335852, 335853, 335854) c6( ) c7(d, pf, at, dL, j, ci, fr, cE, af, ad); #c1(HIF3A) c2(NP_690007) c3(hypoxia-inducible factor 3-alpha isoform a) c4(244741) c5(244742, 244743, 244744, 244745, 244746, 244747) c6( ) c7(dA, hQ, aq, fr, K, B); #c1(HIF3A) c2(NP_690008) c3(hypoxia-inducible factor 3-alpha isoform c) c4(244748, 244749, 244750, 244751, 244752) c5(244753, 244754, 244755, 244756, 244757, 244758, 244759, 244760, 244761, 244762, 244763, 244764, 244765, 244766, 244767, 244768, 244769, 244770, 244771, 244772, 244773, 244774) c6( ) c7(dA, hQ, aq, fr, K, B); #c1(HIF3A) c2(NP_690009) c3(hypoxia-inducible factor 3-alpha isoform d) c4(244775) c5(244776, 244777, 244778, 244779, 244780, 244781, 244782, 244783, 244784, 244785, 244786, 244787, 244788, 244789, 244790, 244791, 244792, 244793) c6( ) c7(dA, hQ, aq, fr, K, B); #c1(HIF3A) c2(XP_005259209) c3(hypoxia-inducible factor 3-alpha isoform X2) c4( ) c5(275981, 275982, 275983, 275984, 275985, 275986, 275987, 275988, 275989, 275990, 275991, 275992) c6( ) c7(dA, hl, aq, fr, K, B); #c1(HIF3A) c2(XP_005259210) c3(hypoxia-inducible factor 3-alpha isoform X3) c4(275993) c5(275994, 275995, 275996, 275997, 275998, 275999) c6( ) c7(dA, hQ, aq, fr, K, B); #c1(HIF3A) c2(XP_005259211) c3(hypoxia-inducible factor 3-alpha isoform X1) c4(276000, 276001, 276002) c5(276003, 276004, 276005, 276006, 276007, 276008) c6( ) c7(dA, hQ, aq, fr, K, B); #c1(HIGD1A) c2(NP_001093138) c3(HIG1 domain family member 1A, mitochondrial isoform a) c4(39823, 39824) c5(39825, 39826, 39827, 39828, 39829, 39830, 39831, 39832) c6( ) c7(if, ey, K, qN); #c1(HIGD1A) c2(NP_001093139) c3(HIG1 domain family member 1A, mitochondrial isoform b) c4( ) c5(39833, 39834, 39835, 39836, 39837, 39838, 39839, 39840) c6( ) c7(if, ey, K, qN); #c1(HIGD1C) c2(NP_001103089) c3(HIG1 domain family member (C) c4( ) c5(41833, 41834, 41835, 41836, 41837) c6( ) c7(bL); #c1(HIGD1C) c2(XP_011536950) c3(HIG1 domain family member IC isoform X1) c4(334632) c5(334633) c6( ) c7(bL); #c1(HIGD2A) c2(NP_620175) c3(HIG1 domain family member 2A, mitochondrial) c4(237471, 237472) c5(237473, 237474, 237475, 237476, 237477, 237478, 237479, 237480, 237481, 237482, 237483, 237484) c6( ) c7(aq); #c1(HILPDA) c2(NP_001092256) c3(hypoxia-inducible lipid droplet-associated protein) c4(39459) c5(39460, 39461, 39462, 39463, 39464, 39465, 39466, 39467, 39468, 39469, 39470, 39471) c6(39472, 39473) c7(es, T, d, py, eR, if, fc, r, rX, yu, fr, ci, qN, blv, q); #c1(HINFP) c2(NP_001230188) c3(histone H4 transcription factor isoform 2) c4(86076) c5(86077, 86078, 86079, 86080, 86081, 86082) c6( ) c7(aKO, ad); #c1(HINFP) c2(XP_011541046) c3(histone H4 transcription factor isoform X2) c4(339569) c5(339570) c6( ) c7(akO, ad); #c1(HINFP) c2(XP_011541047) c3(histone H4 transcription factor isoform X1) c4( ) c5(339571, 339572, 339573, 339574, 339575) c6( ) c7(aKO, ad); #c1(HINT1) c2(NP_005331) c3(histidine triad nucleotide-binding protein 1) c4(155050) c5(155051, 155052, 155053, 155054, 155055, 155056) c6( ) c7(wY, aqh, Gv, jx, bhi, oj, aA, bw, aq, aF, bk, bV, f, B, hX); #c1(HINT2) c2(NP_115982) c3(histidine triad nucleotide-binding protein 2, mitochondrial precursor) c4(225096) c5(225097, 225098, 225099, 225100, 225101, 225102) c6( ) c7(B, dA, af); #c1(HIP1) c2(NP_001230127) c3(huntingtin-interacting protein 1 isoform 2) c4(85966) c5(85967, 85968, 85969, 85970, 85971, 85972, 85973, 85974, 85975) c6( ) c7(T, fM, ha, jh, bB, n, ci, nA, aR, ad, rC, aT, ae, B, blw, eZ, hf, bN, rR, eM, bE, X); #c1(HIP1) c2(NP_005329) c3(huntingtin-interacting protein 1 isoform 1) c4(155036) c5(155037, 155038, 155039, 155040, 155041, 155042) c6( ) c7(T, fM, ha, jh, bB, n, ci, nA, aR, ad, rC, aT, ae, B, blw, eZ, hf, bN, rR, eM, bE, X); #c1(HIP1R) c2(NP_003950) c3(huntingtin-interacting protein 1-related protein isoform 1) c4(142407) c5(142408, 142409, 142410, 142411, 142412, 142413) c6( ) c7(mU, T, Gt, mW, mZ, cj, mS, oh, mY, bK, byQ, an, mV, mT, mX); #c1(HIPK1) c2(XP_011539282) c3(homeodomain-interacting protein kinase 1 isoform X7) c4(337340, 337341) c5(337342, 337343, 337344, 337345, 337346, 337347, 337348, 337349, 337350, 337351, 337352, 337353) c6( ) c7(T, q, K, r); #c1(HIPK2) c2(NP_001106710) c3(homeodomain-interacting protein kinase 2 isoform 2) c4(42609) c5(42610, 42611, 42612, 42613, 42614, 42615) c6( ) c7(bL, T, AS, bB, q, br, eW, hd, Rq, aT, h, K, pi, aW, r, as, t, cm, bw, Hp, ck, qJ, bF, nB, bE, dy, m, Hb, bt, KS, B, jr); #c1(HIPK2) c2(NP_073577) c3(homeodomain-interacting protein kinase 2 isoform 1) c4(212931, 212932) c5(212933, 212934, 212935, 212936, 212937, 212938, 212939, 212940, 212941, 212942, 212943, 212944) c6( ) c7(bL, T, AS, bB, q, br, eW, hd, Rq, aT, h, K, pi, aW, r, as, t, cm, bw, Hp, ck, qJ, bF, nB, bE, dy, m, Hb, bt, kS, B, jr); #c1(HIPK3) c2(NP_001265092) c3(homeodomain-interacting protein kinase 3 isoform 2) c4(97757) c5(97758, 97759, 97760, 97761, 97762, 97763, 97764, 97765, 97766, 97767, 97768, 97769, 97770, 97771, 97772) c6( ) c7(bL, cF, ad, KH); #c1(HIPK3) c2(XP_011518137) c3(homeodomain-interacting protein kinase 3 isoform X1) c4(312115, 312116) c5(312117, 312118, 312119, 312120, 312121, 312122, 312123, 312124, 312125, 312126, 312127) c6( ) c7(bL, cF, ad, kH); #c1(HIRA) c2(NP_003316) c3(protein HIRA) c4(137417, 137418, 137419, 137420, 137421) c5(137422, 137423, 137424, 137425, 137426, 137427, 137428, 137429, 137430, 137431, 137432, 137433, 137434, 137435, 137436, 137437, 137438, 137439, 137440, 137441, 137442, 137443, 137444, 137445, 137446, 137447, 137448, 137449, 137450, 137451) c6( ) c7(bL, agd, RX, bwE, Mz, jy); #c1(HIRIP3) c2(NP_003600) c3(HIRA-interacting protein 3 isoform 1) c4(139488, 139489) c5(139490, 139491, 139492, 139493, 139494, 139495, 139496, 139497, 139498, 139499, 139500, 139501) c6( ) c7(bN); #c1(HIST1H1A) c2(NP_005316) c3(histone H1/) c4(154945) c5(154946, 154947, 154948, 154949, 154950, 154951) c6( ) c7(blx); #c1(HIST1H1B) c2(NP_005313) c3(histone H1.5) c4(154905) c5(154906, 154907, 154908, 154909, 154910, 154911) c6( ) c7(hE, jx, eZ); #c1(HIST1H1C) c2(NP_005310) c3(histone H1.2) c4(154884) c5(154885, 154886, 154887, 154888, 154889, 154890, 154891, 154892, 154893, 154894, 154895, 154896) c6( ) c7(ck, bt, an, aT); #c1(HIST1H1D) c2(NP_005311) c3(histone H1.3) c4(154897) c5( ) c6( ) c7(xb); #c1(HIST1H1E) c2(NP_005312) c3(histone H1.4) c4(154898) c5(154899, 154900, 154901, 154902, 154903, 154904) c6( ) c7(qK); #c1(HIST1H1T) c2(NP_005314) c3(histone H1t) c4(154912) c5(154913, 154914, 154915, 154916, 154917, 154918) c6( ) c7(ck, oe); #c1(HIST1H2AE) c2(NP_066390) c3(histone H2A type 1-B/E) c4(207291) c5(207292, 207293, 207294, 207295, 207296, 207297, 207298, 207299, 207300, 207301, 207302, 207303, 207304, 207305, 207306, 207307) c6( ) c7(cj); #c1(HIST1H2AH) c2(NP_542163) c3(histone H2A type 1-H) c4(231470, 231471) c5(231472, 231473, 231474, 231475, 231476, 231477, 231478, 231479, 231480, 231481, 231482, 231483) c6( ) c7(LB); #c1(HIST1H2BG) c2(NP_003509) c3(histone H2B type 1-C/E/F/G/I) c4(138805) c5( ) c6( ) c7(ad); #c1(HIST1H2BH) c2(NP_003515) c3(histone H2B type 1-H) c4(138813) c5( ) c6( ) c7(ac); #c1(HIST1H2BM) c2(NP_003512) c3(histone H2B type 1-M) c4(138806) c5(138807, 138808, 138809, 138810, 138811, 138812) c6( ) c7(aW, it, aT); #c1(HIST1H3G) c2(NP_003525) c3(histone H3.1) c4(138814) c5(138815, 138816, 138817, 138818, 138819, 138820, 138821, 138822, 138823, 138824, 138825, 138826, 138827, 138828, 138829, 138830, 138831, 138832, 138833, 138834, 138835, 138836, 138837, 138838, 138839, 138840, 138841, 138842, 138843, 138844, 138845, 138846, 138847, 138848, 138849, 138850, 138851, 138852, 138853, 138854, 138855, 138856, 138857, 138858, 138859, 138860) c6( ) c7(d, eA); #c1(HIST1H4D) c2(NP_003530) c3(histone H4) c4(138861) c5(138862, 138863, 138864, 138865, 138866, 138867, 138868, 138869, 138870, 138871, 138872, 138873, 138874, 138875, 138876, 138877, 138878, 138879, 138880, 138881, 138882, 138883, 138884, 138885, 138886, 138887, 138888, 138889, 138890, 138891, 138892, 138893, 138894, 138895, 138896, 138897, 138898, 138899, 138900, 138901, 138902, 138903, 138904, 138905, 138906, 138907, 138908, 138909, 138910, 138911, 138912, 138913, 138914, 138915, 138916, 138917, 138918, 138919, 138920, 138921, 138922, 138923, 138924, 138925, 138926, 138927, 138928, 138929, 138930, 138931, 138932, 138933, 138934, 138935, 138936, 138937, 138938, 138939, 138940, 138941, 138942, 138943, 138944, 138945, 138946, 138947, 138948, 138949, 138950, 138951, 138952, 138953, 138954, 138955, 138956, 138957, 138958, 138959, 138960, 138961, 138962, 138963) c6(138964, 138965, 138966, 138967, 138968, 138969, 138970, 138971, 138972, 138973, 138974, 138975, 138976, 138977, 138978, 138979) c7(Qi, T, ft, eM, iU, Y, eD, ff, eZ, r, rx, eA, en, pb, it, B); #c1(HIST3H3) c2(NP_003484) c3(histone H3.1t) c4(138706) c5(138707, 138708, 138709, 138710, 138711, 138712) c6( ) c7(fq, ad, nA); #c1(HIVEP1) c2(XP_011512855) c3(zinc finger protein 40 isoform X1) c4(303928, 303929, 303930, 303931) c5(303932, 303933, 303934, 303935, 303936, 303937, 303938, 303939, 303940, 303941, 303942, 303943, 303944, 303945, 303946, 303947, 303948, 303949, 303950, 303951, 303952, 303953, 303954, 303955, 303956) c6( ) c7(T, ac, aW, FO, xs, FP, aT, ad); #c1(HIVEP1) c2(XP_011512856) c3(zinc finger protein 40 isoform X4) c4(303957) c5(303958) c6( ) c7(T, ac, aW, FO, xs, FP, aT, ad); #c1(HIVEP1) c2(XP_011512857) c3(zinc finger protein 40 isoform X5) c4(303959) c5(303960, 303961, 303962, 303963, 303964, 303965, 303966, 303967, 303968, 303969, 303970, 303971) c6( ) c7(T, ac, aW, FO, xs, FP, aT, ad); #c1(HIVEP2) c2(NP_006725) c3(transcription factor HIVEP2) c4(167446) c5(167447, 167448, 167449, 167450, 167451, 167452, 167453, 167454, 167455, 167456, 167457, 167458, 167459, 167460, 167461, 167462, 167463) c6( ) c7(aW, j, an, aT); #c1(HJURP) c2(NP_001269891) c3(Holliday junction recognition protein isoform b) c4(103074) c5(103075, 103076, 103077, 103078, 103079, 103080) c6( ) c7(aW, ey, aT); #c1(HJURP) c2(NP_001269892) c3(Holliday junction recognition protein isoform c) c4(103081) c5(103082, 103083, 103084, 103085, 103086, 103087, 103088) c6( ) c7(aW, ey, aT); #c1(HJURP) c2(NP_060880) c3(Holliday junction recognition protein isoform a) c4(198306) c5(198307, 198308, 198309, 198310, 198311, 198312) c6( ) c7(aW, ey, aT); #c1(HK1) c2(NP_000179) c3(hexokinase-1 isoform HK1) c4(3875, 3876) c5(3877, 3878, 3879, 3880, 3881, 3882, 3883, 3884, 3885, 3886, 3887, 3888) c6( ) c7(bL, T, Am, aA, v, blA, j, blz, ai, aR, ad, eA, dP, av, pN, oW, dV, K, j, bq, ae, eG, xX, d, Y, yL, aF, S, nC, bz, bly, VJ, ql, eM, dB, lp, lM); #c1(HK1) c2(NP_277031) c3(hexokinase-1 isoform HK1-R) c4(228745, 228746) c5(228747, 228748, 228749, 228750, 228751, 228752, 228753, 228754, 228755, 228756, 228757, 228758) c6( ) c7(bL, T, Am, aA, v, blA, j, blz, ai, aR, ad, eA, dP, av, pN, oW, dV, K, j, bq, ae, eG, xX, d, Y, yL, aF, S, nC, bz, bly, VJ, ql, eM, dB, lp, lM); #c1(HK1) c2(XP_005269793) c3(hexokinase-1 isoform X2) c4(282826, 282827, 282828) c5(282829, 282830, 282831, 282832, 282833, 282834, 282835, 282836, 282837, 282838, 282839, 282840, 282841, 282842, 282843, 282844, 282845, 282846, 282847, 282848) c6( ) c7(bL, T, Am, aA, v, blA, j, blz, ai, aR, ad, eA, dP, av, pN, oW, dV, K, j, bq, ae, eG, xX, d, Y, yL, aF, S, nC, bz, bly, VJ, ql, eM, dB, lp, lM); #c1(HK1) c2(XP_011538034) c3(hexokinase-1 isoform X1) c4(335597, 335598) c5(335599, 335600, 335601, 335602, 335603, 335604, 335605, 335606, 335607, 335608) c6( ) c7(bL, T, Am, aA, v, blA, j, blz, ai, aR, ad, eA, dP, av, pN, oW, dV, K, j, bq, ae, eG, xX, d, Y, yL, aF, S, nC, bz, bly, VJ, ql, eM, dB, lp, lM); #c1(HK2) c2(NP_000180) c3(hexokinase-2) c4(3889, 3890) c5(3891, 3892, 3893, 3894, 3895, 3896, 3897, 3898, 3899, 3900, 3901, 3902, 3903, 3904, 3905, 3906) c6( ) c7(bL, HQ, T, aC, bB, n, aj, v, j, ci, fr, cd, J, ad, eA, av, jz, aE, aT, jy, ae, d, B, dH, aW, Y, anx, aq, Ej, t, cm, bz, dB, bE, mf, m, im, X, BA); #c1(HK3) c2(NP_002106) c3(hexokinase-3) c4(124605) c5(124606, 124607, 124608, 124609, 124610, 124611) c6( ) c7(bt, j, hl); #c1(HKDC1) c2(NP_079406) c3(putative hexokinase HKDC1) c4(217952, 217953, 217954) c5(217955, 217956, 217957, 217958, 217959, 217960, 217961, 217962, 217963, 217964, 217965, 217966) c6( ) c7(cm, T, bz); #c1(HKDC1) c2(XP_011538497) c3(putative hexokinase HKDC1 isoform X1) c4( ) c5(336249, 336250, 336251, 336252, 336253, 336254) c6( ) c7(cm, T, bz); #c1(HLA-A) c2(NP_001229687) c3(HLA class 1 histocompatibility antigen, A-1 alpha chain precursor) c4(84714, 84715, 84716, 84717, 84718) c5(84719, 84720, 84721, 84722, 84723, 84724, 84725, 84726, 84727, 84728, 84729, 84730, 84731, 84732, 84733, 84734, 84735, 84736, 84737, 84738, 84739, 84740, 84741, 84742, 84743, 84744, 84745, 84746, 84747, 84748, 84749, 84750, 84751, 84752, 84753, 84754, 84755, 84756, 84757, 84758, 84759, 84760, 84761, 84762, 84763, 84764, 84765, 84766, 84767, 84768, 84769, 84770, 84771, 84772, 84773, 84774, 84775, 84776, 84777, 84778, 84779, 84780, 84781, 84782, 84783, 84784, 84785, 84786) c6( ) c7(T, fd, aoj, ci, aY, xy, aE, rw, dV, bBp, hu, hH, cp, aa, uY, lk, GR, fT, Jr, cj, bAD, k, vF, eg, ul, qm, blK, blB, vE, ht, bID, aj, qW, cd, tD, ey, nP, mR, ge, fe, CO, dB, eD, aCD, abW, jv, fZ, aA, q, bIN, yu, rz, DX, arl, bf, lC, lo, pb, cT, OW, ac, iM, ig, iQ, CP, aDR, bt, Ze, iW, aZU, jh, aHv, bIM, ad, wD, hX, vJ, Ot, akJ, oW, lQ, blE, atJ, ff, iV, bz, lU, amS, Om, aDJ, oQ, i, lf, lC, WX, ne, rk, bM, xS, if, bB, cL, bh, ip, x, vV, fS, bFU, ja, azm, bc, Jl, eE, eF, cm, xD, blQ, aiM, hh, avY, bIG, oY, WB, RN, bsg, UD, eV, DU, cC, A, lb, yq, jq, bP, eo, fL, B, dA, eX, V, ld, ae, hM, ev, afq, bu, aaO, t, ck, yH, dM, iD, qi, od, afm, ds, qh, hD, rN, vc, JK, bsn, nJ, atX, d, je, wc, bHX, CF, gk, yE, y, sH, pC, aC, fR, hV, al, qj, lR, aMB, wa, AW, rB, wt, DY, mH, aEV, blP, axu, m, aGQ, hN, g, ve, uC, iP, dW, aDM, bsh, Ro, QO, OI, fN, apt, aUX, ft, kH, aaA, blO, dx, aXy, bIL, cM, Zj, bIF, bIH, W, bbx, aZL, mg, JM, Xa, blR, fM, fG, jx, hz, j, pd, eA, J, o, er, lg, hK, kl, Y, r, en, aoG, iK, bN, sn, Dy, kB, hA, hc, Da, dy, bGB, gT, LS, GI, aaT, aFe, aZ, ah, hF, dP, acu, io, aNp, Yi, lU, BN, eH, cr, hp, Fq, Pd, iR, TA, Bv, Lj, aCl, hE, eb, bwS, aU, pk, aW, HA, aVc, bdc, eM, iT, aFc, fr, qN, TI, aPb, ai, eW, bBq, h, dL, aDC, ux, hB, aq, gG, kk, Wt, Qu, hk, bm, uz, aDE, bL, bll, eR, dK, rf, blC, mP, qp, aT, af, aZQ, blJ, iU, yL, aDm, f, lY, ajl, BU, yW, Zg, AH, Kd, qb, aD, jE, mC, qC, av, F, aal, oz, azq, vO, yF, ul, eZ, yy, Ej, af, LB, axs, bE, aED, fh, X, Ex); #c1(HLA-A) c2(NP_002107) c3(HLA class 1 histocompatibility antigen, A-1 alpha chain precursor) c4(124612, 124613, 124614, 124615, 124616, 124617) c5(124618, 124619, 124620, 124621, 124622, 124623, 124624, 124625, 124626, 124627, 124628, 124629, 124630, 124631, 124632, 124633, 124634, 124635, 124636, 124637, 124638, 124639, 124640, 124641, 124642, 124643, 124644, 124645, 124646, 124647, 124648, 124649, 124650, 124651, 124652, 124653, 124654, 124655, 124656, 124657, 124658, 124659, 124660, 124661, 124662, 124663, 124664, 124665, 124666, 124667, 124668, 124669, 124670, 124671, 124672, 124673, 124674, 124675, 124676, 124677, 124678, 124679, 124680, 124681, 124682, 124683, 124684) c6( )c7(T, fd, aoj, ci, aY, xy, aE, rw, dV, bBp, hu, hH, cp, aa, uY, lk, GR, fT, Jr, cj, bAD, k, vF, eg, uL, qm, blK, blB, vE, ht, blD, aj, qW, cd, tD, ey, nP, mR, ge, fe, CQ, dB, eD, aCD, abW, jv, fZ, aA, q, blN, yu, rz, DX, arl, bf, lC, lo, pb, cT, OW, ac, iM, ig, iQ, CP, aDR, bt, Ze, iW, aZU, jh, aHv, blM, ad, wD, hX, vJ, Ot, akJ, oW, lQ, blE, atJ, ff, iV, bz, lU, amS, Om, aDJ, oQ, i, lf, lC, WX, ne, rk, bM, xS, if, bB, cL, bh, ip, x, vV, fS, bFU, ja, azm, bc, Jl, eE, eF, cm, xD, blQ, aiM, hh, avY, blG, oY, WB, RN, bsg, UD, eV, DU, cC, A, lb, yq, jq, bP, eo, fL, B, dA, ex, V, ld, ae, hM, ev, afq, bu, aaO, t, ck, yH, dM, iD, qi, od, afm, ds, qh, hD, rN, vc, JK, bsn, nJ, atX, d, je, wc, bHX, CF, gk, yE, y, sH, pC, aC, fR, hV, al, qj, lR, aMB, wa, AW, rB, wt, DY, mH, aEV, blP, axu, md, aGQ, hN, g, ve, uC, iP, dW, aDM, bsh, Ro, QO, Ol, fN, apt, aUX, ft, kH, aaA, blO, dx, aXy, blL, cM, Zj, blF, blH, W, bbx, aZL, mg, JM, Xa, blR, fM, fG, jx, hz, j, pd, eA, J, o, er, lg, hK, kl, Y, r, en, aoG, iK, bN, sn, Dy, kB, hA, hc, Da, dy, bGB, gT, LS, Gl, aaT, aFe, aZ, ah, hF, dP, acu, io, aNp, Yi, lU, BN, eH, cr, hp, Fq, Pd, iR, TA, Bv, Lj, aCl, hE, eb, bwS, aU, pk, aW, HA, aVc, bdc, eM, iT, aFc, fr, qN, Tl, aPb, ai, eW, bBq, h, dL, aDC, ux, hB, aq, gG, kk, Wt, Qu, hk, bm, uz, aDE, bL, bll, eR, dk, rf, blC, mP, qp, aT, af, aZQ, blJ, iU, yL, aDm, f, lY, ajl, BU, yW, Zg, AH, Kd, qb, aD, jE, mC, qC, av, F, aa, oz, azq, vO, yF, uQ, eZ, yy, Ej, aF, LB, axs, bE, aED, fh, X, Ex); #c1(HLA-A) c2(XP_011546426) c3(HLA class I histocompatibility antigen, A-1 alpha chain isoform X1) c4(345181, 345182, 345183, 345184, 345185, 345186, 345187, 345188, 345189, 345190, 345191, 345192, 345193, 345194, 345195, 345196, 345197, 345198, 345199, 345200) c5(345201, 345202, 345203, 345204, 345205, 345206, 345207, 345208, 345209, 345210, 345211, 345212, 345213, 345214, 345215, 345216, 345217, 345218, 345219, 345220, 345221, 345222, 345223, 345224, 345225, 345226, 345227, 345228, 345229, 345230, 345231, 345232, 345233, 345234, 345235, 345236, 345237, 345238, 345239, 345240, 345241, 345242, 345243, 345244, 345245, 345246) c6( )c7(T, fd, aoj, ci, aY, xy, aE, rw, dV, bBp, hu, hH, cp, aa, uY, lk, GR, fT, Jr, cj, bAD, k, vF, eg, uL, qm, blK, blB, vE, ht, blD, aj, qW, cd, tD, ey, nP, mR, ge, fe, CQ, dB, eD, aCD, abW, jv, fZ, aA, q, blN, yu, rz, DX, arl, bf, lC, lo, pb, cT, OW, ac, iM, ig, iQ, CP, aDR, bt, Ze, iW, aZU, jh, aHv, blM, ad, wD, hX, vJ, Ot, akJ, oW, lQ, blE, atJ, ff, iV, bz, lU, amS, Om, aDJ, oQ, i, lf, lC, WX, ne, rk, bM, xS, if, bB, cL, bh, ip, x, vV, fS, bFU, ja, azm, bc, Jl, eE, eF, cm, xD, blQ, aiM, hh, avY, blG, oY, WB, RN, bsg, UD, eV, DU, cC, A, lb, yq, jq, bP, eo, fL, B, dA, eX, V, ld, ae, hM, ev, afq, bu, aaO, t, ck, yH, dM, iD, qi, od, afm, ds, qh, hD, rN, vc, JK, bsn, nJ, atX, d, je, wc, bHX, CF, gk, yE, y, sH, pC, aC, fR, hV, al, qj, lR, aMB, wa, AW, rB, wt, DY, mH, aEV, blP, axu, ml, aGQ, hN, g, ve, uC, iP, dW, aDM, bsh, Ro, QO, Ol, fN, apt, aUX, ft, kH, aaA, blO, dx, aXy, blL, cM, Zj, blF, blH, W, bbx, aZL, mg, JM, Xa, blR, fM, fG, jx, hz, j, pd, eA, J, o, er, lg, hK, kl, Y, r, en, aoG, iK, bN, sn, Dy, kB, hA, hc, Da, dy, bGB, gT, LS, Gl, aaT, aFe, aZ, ah, hF, dP, acu, io, aNp, Yi, lU, BN, eH, cr, hp, Fq, Pd, iR, TA, Bv, Lj, aCl, hE, eb, bwS, aU, pk, aW, HA, aVc, bdc, eM, iT, aFc, fr, qN, Tl, aPb, ai, eW, bBq, h, dL, aDC, ux, hB, aq, gG, kk, Wt, Qu, hk, bm, uz, aDE, bL, bll, eR, dk, rf, blC, mP, qp, aT, af, aZQ, blJ, iU, yL, aDm, f, lY, ajl, BU, yW, Zg, AH, Kd, qb, aD, jE, mC, qC, av, F, aaU, oz, azq, vO, yF, uQ, eZ, yy, Ej, aF, LB, axs, bE, aED, fh, X, Ex); #c1(HLA-B) c2(NP_005505) c3(major histocompatibility complex, class l, B precursor) c4(156895, 156896, 156897) c5(156898, 156899, 156900, 156901, 156902, 156903, 156904, 156905, 156906, 156907, 156908, 156909, 156910, 156911) c6( )c7(bM, T, aC, xS, fR, fV, if, e, al, bO, CH, eb, fd, bwS, gA, qj, ci, bh, aoj, aY, Tz, aMB, bv, AW, wD, xy, vV, rw, aX, fS, bFU, dV, ja, aU, azm, rB, K, hu, wt, hH, LG, eL, bc, aiJ, cp, aa, hm, mH, aEV, bG, blP, aW, axu, asD, mQ, HA, LZ, blU, uY, hN, g, eE, ve, eF, aVc, GR, cm, y, fT, iT, Jr, aiM, cj, dW, bAD, k, m, aDM, vF, eg, ul, qR, qm, bsh, cE, blB, QO, vE, yG, ht, fN, pk, blG, n, blD, aj, oY, fr, uE, aUX, bd, qN, qW, cd, ai, eh, ale, eW, aJf, aPb, ft, xC, adX, aMj, kH, h, dl, aaA, blO, aDC, ux, ey, he, blX, rk, nP, OV, mR, eZ, A, MD, aXy, aHW, sp, ff, lb, ge, fe, yq, aMJ, CQ, gG, kk, cM, Zj, lx, Wt, jq, blF, blH, abV, adZ, W, NL, dB, hk, aZL, lf, mg, JM, bm, uz, fL, aCD, DY, Pr, B, abW, it, bL, blR, jv, fM, bll, eR, fG, jx, rf, aA, j, V, q, J, eA, o, ga, aWb, blC, blN, fh, qp, ld, yg, aT, yu, hK, rz, DX, ae, aZQ, blW, hM, bf, iU, Y, lC, r, afq, lo, aDm, en, t, pb, bN, lY, ck, ajl, Ta, OW, sn, c, aQd, hA, ig, nM, iM, AH, iQ, Zg, CP, auT, Da, dy, gT, bt, Kd, aaT, aFe, Ke, aD, dM, iW, ha, jh, aHv, iD, dG, jE, blT, oL, hF, bCx, XB, ad, qC, dP, kL, hX, F, od, av, afm, blV, acu, Ot, ds, azq, qh, oW, akJ, hD, rN, vc, JK, lQ, bsn, yF, u, d, BN, ue, lU, je, KS, wc, azP, bHX, eH, dN, hv, yy, aF, hp, cr, Fq, Pd, iV, dr, aYt, LB, Bv, zr, axs, yE, blS, arE, Ny, bE, aED, Om, Lj, aCl, oQ, i, lC, ra, ne, sH, pC); #c1(HLA-B) c2(XP_005275157) c3(major histocompatibility complex, class I, B isoform X3) c4(285552, 285553, 285554) c5(285555, 285556, 285557, 285558, 285559, 285560, 285561, 285562, 285563, 285564, 285565, 285566, 285567, 285568, 285569, 285570, 285571, 285572, 285573) c6( )c7(bM, T, aC, xS, fR, fV, if, e, al, bO, CH, eb, fd, bwS, gA, qj, ci, bh, aoj, aY, Tz, aMB, bv, AW, wD, xy, vV, rw, aX, fS, bFU, dV, ja, aU, azm, rB, K, hu, wt, hH, LG, eL, bc, aiJ, cp, aa, hm, mH, aEV, bG, blP, aW, axu, asD, mQ, HA, LZ, blU, uY, hN, g, eE, ve, eF, aVc, GR, cm, y, fT, iT, Jr, aiM, cj, dW, bAD, k, m, aDM, vF, eg, uL, qR, qm, bsh, cE, blB, QO, vE, yG, ht, fN, pk, blG, n, blD, aj, oY, fr, uE, aUX, bd, qN, qW, cd, ai, eh, ale, eW, aJf, aPb, ft, xC, adX, aMj, kH, h, dL, aaA, blO, aDC, ux, ey, he, blX, rk, nP, OV, mR, eZ, A, MD, aXy, aHW, sp, ff, lb, ge, fe, yq, aMJ, CQ, gG, kk, cM, Zj, lx, Wt, jq, blF, blH, abV, adZ, W, NL, dB, hk, aZL, lf, mg, JM, bm, uz, fl, aCD, DY, Pr, B, abW, it, bL, blR, jv, fM, bll, eR, fG, jx, rf, aA, j, V, q, J, eA, o, ga, aWb, blC, blN, fh, qp, ld, yg, aT, yu, hK, rz, DX, ae, aZd, blW, hM, bf, iU, Y, lC, r, afq, lo, aDm, en, t, pb, bN, lY, ck, ajl, Ta, OW, sn, c, aQd, hA, ig, nM, iM, AH, iQ, Zg, CP, auT, Da, dy, gT, bt, Kd, aaT, aFe, Ke, aD, dM, iW, ha, jh, aHv, iD, dG, JE, blT, oL, hF, bCx, XB, ad, qC, dP, kL, hX, F, od, av, afm, blV, acu, Ot, ds, azq, qh, oW, akJ, hD, rN, vc, JK, lQ, bsn, yF, uQ, d, BN, ue, lU, je, KS, wc, azP, bHX, eH, dN, hv, yy, aF, hp, cr, Fq, Pd, iV, dr, aYt, LB, Bv, zr, axs, yE, blS, arE, Ny, bE, aED, Om, Lj, aCl, od, i, lC, ra, ne, sH, pC); #c1(HLA-B) c2(XP_005275592) c3(major histocompatibility complex, class I, B isoform X6) c4(285628, 285629, 285630) c5(285631, 285632, 285633, 285634, 285635, 285636, 285637, 285638, 285639, 285640, 285641, 285642, 285643, 285644, 285645, 285646, 285647, 285648) c6( )c7(bM, T, aC, xS, fR, fV, if, e, al, bO, CH, eb, fd, bwS, gA, qj, ci, bh, aoj, aY, Tz, aMB, bv, AW, wD, xy, vV, rw, aX, fS, bFU, dV, ja, aU, azm, rB, K, hu, wt, hH, LG, eL, bc, aiJ, cp, aa, hm, mH, aEV, bG, blP, aW, axu, asD, mQ, HA, LZ, blU, uY, hN, g, eE, ve, ef, aVc, GR, cm, y, fT, iT, Jr, aiM, cj, dW, bAD, k, m, aDM, vF, eg, uL, qR, qm, bsh, cE, blB, QO, vE, yG, ht, fN, pk, blG, n, blD, aj, oY, fr, uE, aUX, bd, qN, qW, cd, ai, eh, aJe, eW, aJf, aPb, ft, xC, adX, aMj, kH, h, dL, aaA, blO, aDC, ux, ey, he, blX, rk, nP, OV, mR, eZ, A, MD, aXy, aHW, sp, ff, lb, ge, fe, yq, aMJ, CQ, gG, kk, cM, Zj, lx, Wt, jq, blF, blH, abV, adZ, W, NL, dB, hk, aZL, lf, mg, JM, bm, uz, fl, aCD, DY, Pr, B, abW, it, bL, blR, jv, fM, bll, eR, fG, jx, rf, aA, j, V, q, J, eA, o, ga, aWb, blC, blN, fh, qp, ld, yg, aT, yu, hK, rz, DX, ae, aZd, blW, hM, bf, il, Y, lC, r, afq, lo, aDm, en, t, pb, bN, lY, ck, ajl, Ta, OW, sn, c, ald, hA, ig, nM, iM, AH, il, Zg, CP, auT, Da, dy, gT, bt, Kd, aaT, aFe, Ke, aD, dM, iW, ha, jh, aHv, iD, dG, jE, blT, oL, hF, bCx, XB, ad, qC, dP, kL, hX, F, od, av, afm, blV, acu, Ot, ds, azq, qh, oW, akJ, hD, rN, vc, JK, lQ, bsn, yF, uQ, d, BN, ue, lU, je, KS, wc, azP, bHX, eH, dN, hv, yy, aF, hp, cr, Fq, Pd, iV, dr, aYt, LB, Bv, zr, axs, yE, blS, arE, Ny, bE, aED, Om, Lj, aCl, oQ, i, lC, ra, ne, sH, pC); #c1(HLA-B) c2(XP_006725968) c3(major histocompatibility complex, class I, B isoform X5) c4(297020, 297021, 297022) c5(297023, 297024, 297025, 297026, 297027, 297028, 297029, 297030, 297031, 297032, 297033, 297034, 297035, 297036) c6( )c7(bM, T, aC, xS, fR, fV, if, e, al, bO, CH, eb, fd, bwS, gA, qj, ci, bh, aoj, aY, Tz, aMB, bv, AW, wD, xy, vV, rw, aX, fS, bFU, dV, ja, aU, azm, rB, K, hu, wt, hH, LG, eL, bc, aiJ, cp, aa, hm, mH, aEV, bG, blP, aW, axu, asD, mQ, HA, LZ, blU, uY, hN, g, eE, ve, eF, aVc, GR, cm, y, fT, iT, Jr, aiM, cj, dW, bAD, k, m, aDM, vF, eg, uL, qR, qm, bsh, cE, blB, QO, vE, yG, ht, fN, pk, blG, n, blD, aj, oY, fr, uE, aUX, bd, qN, qW, cd, ai, eh, ale, eW, aJf, aPb, ft, xC, adX, aMj, kH, h, dL, aaA, blO, aDC, ux, ey, he, blX, rk, nP, OV, mR, eZ, A, MD, aXy, aHW, sp, ff, lb, ge, fe, yq, aMJ, CQ, gG, kk, cM, Zj, lx, Wt, jq, blF, blH, abV, adZ, W, NL, dB, hk, aZL, lf, mg, JM, bm, uz, fl, aCD, DY, Pr, B, abW, it, bL, blR, jv, fM, bll, eR, fG, jx, rf, aA, j, V, q, J, eA, o, ga, aWb, blC, blN, fh, qp, ld, yg, aT, yu, hK, rz, DX, ae, aZQ, blW, hM, bf, iU, Y, lC, r, afq, lo, aDm, en, t, pb, bN, lY, ck, ajl, Ta, OW, sn, c, aQd, hA, ig, nM, iM, AH, iQ, Zg, CP, auT, Da, dy, gT, bt, Kd, aaT, aFe, Ke, aD, dM, iW, ha, jh, aHv, iD, dG, jE, blT, oL, hF, bCx, XB, ad, qC, dP, kL, hX, F, od, av, afm, blV, acu, Ot, ds, azq, qh, oW, akJ, hD, rN, vc, JK, lQ, bsn, yF, uQ, d, BN, ue, lU, je, KS, wc, azP, bHX, eH, dN, hv, yy, aF, hp, cr, Fq, Pd, iV, dr, aYt, LB, Bv, zr, axs, yE, blS, arE, Ny, bE, aED, Om, Lj, aCl, oQ, i, lC, ra, ne, sH, pC); #c1(HLA-B) c2(XP_011512858) c3(major histocompatibility complex, class I, B isoform X1) c4(303972) c5( )c6( )c7(bM, T, aC, xS, fR, fV, if, e, al, bO, CH, eb, fd, bwS, gA, qj, ci, bh, aoj, aY, Tz, aMB, bv, AW, wD, xy, vV, rw, aX, fS, bFU, dV, ja, aU, azm, rB, K, hu, wt, hH, LG, eL, bc, aiJ, cp, aa, hm, mH, aEV, bG, blP, aW, axu, asD, md, HA, LZ, blU, uY, hN, g, eE, ve, eF, aVc, GR, cm, y, fT, iT, Jr, aiM, cj, dW, bAD, k, m, aDM, vF, eg, uL, qR, qm, bsh, cE, blB, QO, vE, yG, ht, fN, pk, blG, n, blD, aj, oY, fr, uE, aUX, bd, qN, qW, cd, ai, ch, aJe, eW, aJf, aPb, ft, xC, adX, aMj, kH, h, dL, aaA, blO, aDC, ux, ey, he, blX, rk, nP, OV, mR, eZ, A, MD, aXy, aHW, sp, ff, lb, ge, fe, yq, aMJ, CQ, gG, kk, cM, Zj, lx, Wt, jq, blF, blH, abV, adZ, W, NL, dB, hk, aZL, lf, mg, JM, bm, uz, fl, aCD, DY, Pr, B, abW, it, bL, blR, jv, fM, bll, eR, fG, jx, rf, aA, j, V, q, J, eA, o, ga, aWb, blC, blN, fh, qp, ld, yg, aT, yu, hK, rz, DX, ae, aZQ, blW, hM, bf, iU, Y, lC, r, afq, lo, aDm, en, t, pb, bN, lY, ck, ajl, Ta, OW, sn, c, aQd, hA, ig, nM, iM, AH, iQ, Zg, CP, auT, Da, dy, gT, bt, Kd, aaT, aFe, Ke, aD, dM, iW, ha, jh, aHv, iD, dG, jE, blT, oL, hF, bCx, XB, ad, qC, dP, kL, hX, F, od, av, afm, blV, acu, Ot, ds, azq, qh, oW, akJ, hD, rN, vc, JK, lQ, bsn, yF, uQ, d, BN, ue, lU, je, KS, wc, azP, bHX, eH, dN, hv, yy, aF, hp, cr, Fq, Pd, iV, dr, aYt, LB, Bv, zr, axs, yE, blS, arE, Ny, bE, aED, Om, Lj, aCl, oQ, i, lC, ra, ne, sH, pC); #c1(HLA-B) c2(XP_011512859) c3(major histocompatibility complex, class I, B isoform X7) c4(303973) c5(303974, 303975, 303976, 303977, 303978, 303979, 303980, 303981, 303982, 303983, 303984, 303985, 303986, 303987, 303988, 303989, 303990, 303991, 303992, 303993, 303994, 303995, 303996, 303997, 303998, 303999, 304000, 304001, 304002, 304003, 304004, 304005, 304006, 304007) c6( )c7(bM, T, aC, xS, fR, fV, if, e, al, bO, CH, eb, fd, bwS, gA, qj, ci, bh, aoj, aY, Tz, aMB, bv, AW, wD, xy, vV, rw, aX, fS, bFU, dV, ja, aU, azm, rB, K, hu, wt, hH, LG, eL, bc, aiJ, cp, aa, hm, mH, aEV, bG, blP, aW, axu, asD, mQ, HA, LZ, blU, uY, hN, g, eE, ve, eF, aVc, GR, cm, y, fT, iT, Jr, aiM, cj, dW, bAD, k, m, aDM, vF, eg, uL, qR, qm, bsh, cE, blB, QO, vE, yG, ht, fN, pk, blG, n, blD, aj, oY, fr, uE, aUX, bd, qN, qW, cd, ai, eh, aJe, eW, aJf, aPb, ft, xC, adX, aMj, kH, h, dL, aaA, blO, aDC, ux, ey, he, blX, rk, nP, OV, mR, eZ, A, MD, aXy, aHW, sp, ff, lb, ge, fe, yq, aMJ, CQ, gG, kk, cM, Zj, lx, Wt, jq, blF, blH, abV, adZ, W, NL, dB, hk, aZL, lf, mg, JM, bm, uz, fL, aCD, DY, Pr, B, abW, it, bL, blR, jv, fM, bll, eR, fG, jx, rf, aA, j, V, q, J, eA, o, ga, aWb, blC, blN, fh, qp, ld, yg, aT, yu, hK, rz, DX, ae, aZQ, blW, hM, bf, iU, Y, lC, r, afq, lo, aDm, en, t, pb, bN, lY, ck, ajl, Ta, OW, sn, c, aQd, hA, ig, nM, iM, AH, iQ, Zg, CP, auT, Da, dy, gT, bt, Kd, aaT, aFe, Ke, aD, dM, iW, ha, jh, aHv, iD, dG, jE, blT, oL, hF, bCx, XB, ad, qC, dP, kL, hX, F, od, av, afm, blV, acu, Ot, ds, azq, qh, oW, akJ, hD, rN, vc, JK, lQ, bsn, yF, uQ, d, BN, ue, lU, je, KS, wc, azP, bHX, eH, dN, hv, yy, aF, hp, cr, Fq, Pd, iV, dr, aYt, LB, Bv, zr, axs, yE, blS, arE, Ny, bE, aED, Om, Lj, aCl, oQ, i, lC, ra, ne, sH, pC); #c1(HLA-B) c2(XP_011545539) c3(major histocompatibility complex, class I, B isoform X1) c4(344218, 344219) c5( )c6( )c7(bM, T, aC, xS, fR, fV, if, e, al, bO, CH, eb, fd, bwS, gA, qj, ci, bh, aoj, aY, Tz, aMB, bv, AW, wD, xy, vV, rw, aX, fS, bFU, dV, ja, aU, azm, rB, K, hu, wt, hH, LG, eL, bc, aiJ, cp, aa, hm, mH, aEV, bG, blP, aW, axu, asD, mQ, HA, LZ, blU, uY, hN, g, eE, ve, eF, aVc, GR, cm, y, fT, iT, Jr, aiM, cj, dW, bAD, k, m, aDM, vF, eg, ul, qR, qm, bsh, cE, blB, QO, vE, yG, ht, fN, pk, blG, n, blD, aj, oY, fr, uE, aUX, bd, qN, qW, cd, ai, eh, ale, eW, aJf, aPb, ft, xC, adX, aMj, kH, h, dL, aaA, blO, aDC, ux, ey, he, blX, rk, nP, OV, mR, eZ, A, MD, aXy, aHW, sp, ff, lb, ge, fe, yq, aMJ, CQ, gG, kk, cM, Zj, lx, Wt, jq, blF, blH, abV, adZ, W, NL, dB, hk, aZL, lf, mg, JM, bm, uz, fl, aCD, DY, Pr, B, abW, it, bL, blR, jv, fM, bll, eR, fG, jx, rf, aA, j, V, q, J, eA, o, ga, aWb, blC, blN, fh, qp, ld, yg, aT, yu, hK, rz, DX, ae, aZQ, blW, hM, bf, iU, Y, lC, r, afq, lo, aDm, en, t, pb, bN, lY, ck, ajl, Ta, OW, sn, c, ald, hA, ig, nM, iM, AH, iQ, Zg, CP, auT, Da, dy, gT, bt, Kd, aaT, aFe, Ke, aD, dM, iW, ha, jh, aHv, iD, dG, jE, blT, oL, hF, bCx, XB, ad, qC, dP, kL, hX, F, od, av, afm, blV, acu, Ot, ds, azq, qh, oW, akJ, hD, rN, vc, JK, lQ, bsn, yF, uQ, d, BN, ue, lU, je, KS, wc, azP, bHX, eH, dN, hv, yy, aF, hp, cr, Fq, Pd, iV, dr, aYt, LB, Bv, zr, axs, yE, blS, arE, Ny, bE, aED, Om, Lj, aCl, oQ, i, lC, ra, ne, sH, pC); #c1(HLA-B) c2(XP_011545540) c3(major histocompatibility complex, class I, B isoform X2) c4(344220, 344221, 344222) c5(344223, 344224, 344225, 344226, 344227, 344228, 344229, 344230, 344231, 344232, 344233, 344234, 344235, 344236, 344237, 344238, 344239, 344240, 344241) c6( )c7(bM, T, aC, xS, fR, fV, if, e, al, bO, CH, eb, fd, bwS, gA, qj, ci, bh, aoj, aY, Tz, aMB, bv, AW, wD, xy, vV, rw, aX, fS, bFU, dV, ja, aU, azm, rB, K, hu, wt, hH, LG, eL, bc, aiJ, cp, aa, hm, mH, aEV, bG, blP, aW, axu, asD, mQ, HA, LZ, blU, uY, hN, g, eE, ve, eF, aVc, GR, cm, y, fT, iT, Jr, aiM, cj, dW, bAD, k, m, aDM, vF, eg, uL, qR, qm, bsh, cE, blB, QO, vE, yG, ht, fN, pk, blG, n, blD, aj, oY, fr, uE, aUX, bd, qN, qW, cd, ai, eh, aJe, eW, aJf, aPb, ft, xC, adX, aMj, kH, h, dL, aaA, blO, aDC, ux, ey, he, blX, rk, nP, OV, mR, eZ, A, MD, aXy, aHW, sp, ff, lb, ge, fe, yq, aMJ, CQ, gG, kk, cM, Zj, lx, Wt, jq, blF, blH, abV, adZ, W, NL, dB, hk, aZL, lf, mg, JM, bm, uz, fl, aCD, DY, Pr, B, abW, it, bL, blR, jv, fM, bll, eR, fG, jx, rf, aA, j, V, q, J, eA, o, ga, aWb, blC, blN, fh, qp, ld, yg, aT, yu, hK, rz, DX, ae, aZQ, blW, hM, bf, iU, Y, lC, r, afq, lo, aDm, en, t, pb, bN, lY, ck, ajl, Ta, OW, sn, c, aQd, hA, ig, nM, iM, AH, iQ, Zg, CP, auT, Da, dy, gT, bt, Kd, aaT, aFe, Ke, aD, dM, iW, ha, jh, aHv, iD, dG, JE, blT, oL, hF, bCx, XB, ad, qC, dP, kL, hX, F, od, av, afm, blV, acu, Ot, ds, azq, qh, oW, akJ, hD, rN, vc, JK, lQ, bsn, yF, uQ, d, BN, ue, lU, je, KS, wc, azP, bHX, eH, dN, hv, yy, aF, hp, cr, Fq, Pd, iV, dr, aYt, LB, Bv, zr, axs, yE, blS, arE, Ny, bE, aED, Om, Lj, aCl, oQ, i, lC, ra, ne, sH, pC); #c1(HLA-B) c2(XP_011545541) c3(major histocompatibility complex, class l, B isoform X7) c4( )c5(344242, 344243, 344244, 344245, 344246, 344247, 344248, 344249, 344250, 344251, 344252) c6( )c7(bM, T, aC, xS, fR, fV, if, e, al, bO, CH, eb, fd, bwS, gA, qj, ci, bh, aoj, aY, Tz, aMB, bv, AW, wD, xy, vV, rw, aX, fS, bFU, dV, ja, aU, azm, rB, K, hu, wt, hH, LG, eL, bc, aiJ, cp, aa, hm, mH, aEV, bG, blP, aW, axu, asD, mQ, HA, LZ, blU, uY, hN, g, eE, ve, eF, aVc, GR, cm, y, fT, iT, Jr, aiM, cj, dW, bAD, k, m, aDM, vF, eg, ul, qR, qm, bsh, cE, blB, QO, vE, yG, ht, fN, pk, blG, n, blD, aj, oY, fr, uE, aUX, bd, qN, qW, cd, ai, eh, aJe, eW, aJf, aPb, ft, xC, adX, aMj, kH, h, dl, aaA, blO, aDC, ux, ey, he, blX, rk, nP, OV, mR, eZ, A, MD, aXy, aHW, sp, ff, lb, ge, fe, yq, aMJ, CQ, gG, kk, cM, Zj, lx, Wt, jq, blF, blH, abV, adZ, W, NL, dB, hk, aZL, lf, mg, JM, bm, uz, fL, aCD, DY, Pr, B, abW, it, bL, blR, jv, fM, bll, eR, fG, jx, rf, aA, j, V, q, J, eA, o, ga, aWb, blC, blN, fh, qp, ld, yg, aT, yu, hK, rz, DX, ae, aZQ, blW, hM, bf, iU, Y, lC, r, afq, lo, aDm, en, t, pb, bN, lY, ck, ajl, Ta, OW, sn, c, aQd, hA, ig, nM, iM, AH, iQ, Zg, CP, auT, Da, dy, gT, bt, Kd, aaT, aFe, Ke, aD, dM, iW, ha, jh, aHv, iD, dG, jE, blT, oL, hF, bCx, XB, ad, qC, dP, kL, hX, F, od, av, afm, blV, acu, Ot, ds, azq, qh, oW, akJ, HD, rN, vc, JK, lQ, bsn, yF, uQ, d, BN, ue, lU, je, KS, wc, azP, bHX, eH, dN, hv, yy, aF, hp, cr, Fq, Pd, iV, dr, aYt, LB, Bv, zr, axs, yE, blS, arE, Ny, bE, aED, Om, Lj, aCl, oQ, i, lC, ra, ne, sH, pC); #c1(HLA-B) c2(XP_011546175) c3(major histocompatibility complex, class I, B isoform X4) c4(344866, 344867, 344868, 344869) c5(344870, 344871, 344872) c6( )c7(bM, T, aC, xS, fR, fV, if, e, al, bO, CH, eb, fd, bwS, gA, qj, ci, bh, aoj, aY, Tz, aMB, bv, AW, wD, xy, vV, rw, aX, fS, bFU, dV, ja, aU, azm, rB, K, hu, wt, hH, LG, eL, bc, aiJ, cp, aa, hm, mH, aEV, bG, blP, aW, axu, asD, mQ, HA, LZ, blU, uY, hN, g, eE, ve, eF, aVc, GR, cm, y, fT, iT, Jr, aiM, cj, dW, bAD, k, m, aDM, vF, eg, ul, qR, qm, bsh, cE, blB, QO, vE, yG, ht, fN, pk, blG, n, blD, aj, oY, fr, uE, aUX, bd, qN, qW, cd, ai, eh, aJe, eW, aJf, aPb, ft, xC, adX, aMj, kH, h, dL, aaA, blO, aDC, ux, ey, he, blX, rk, nP, OV, mR, eZ, A, MD, aXy, aHW, sp, ff, lb, ge, fe, yq, aMJ, CQ, gG, kk, cM, Zj, lx, Wt, jq, blF, blH, abV, adZ, W, NL, dB, hk, aZL, lf, mg, JM, bm, uz, fL, aCD, DY, Pr, B, abW, it, bL, blR, jv, fM, bll, eR, fG, jx, rf, aA, j, V, q, J, eA, o, ga, aWb, blC, blN, fh, qp, ld, yg, aT, yu, hK, rz, DX, ae, aZQ, blW, hM, bf, iU, Y, lC, r, afq, lo, aDm, en, t, pb, bN, lY, ck, ajl, Ta, OW, sn, c, aQd, hA, ig, nM, iM, AH, iQ, Zg, CP, auT, Da, dy, gT, bt, Kd, aaT, aFe, Ke, aD, dM, iW, ha, jh, aHv, iD, dG, jE, blT, oL, hF, bCx, XB, ad, qC, dP, kL, hX, F, od, av, afm, blV, acu, Ot, ds, azq, qh, oW, akJ, hD, rN, vc, JK, lQ, bsn, yF, uQ, d, BN, ue, lU, je, KS, wc, azP, bHX, eH, dN, hv, yy, af, hp, cr, Fq, Pd, iV, dr, aYt, LB, Bv, zr, axs, yE, blS, arE, Ny, bE, aED, Om, Lj, aCl, oQ, i, lC, ra, ne, sH, pC); #c1(HLA-B) c2(XP_011546176) c3(major histocompatibility complex, class I, B isoform X7) c4( )c5(344873, 344874, 344875, 344876, 344877, 344878, 344879, 344880, 344881, 344882) c6( )c7(bM, T, aC, xS, fR, fV, if, e, al, bO, CH, eb, fd, bwS, gA, qj, ci, bh, aoj, aY, Tz, aMB, bv, AW, wD, xy, vV, rw, aX, fS, bFU, dV, ja, aU, azm, rB, K, hu, wt, hH, LG, eL, bc, aiJ, cp, aa, hm, mH, aEV, bG, blP, aW, axu, asD, mQ, HA, LZ, blU, uY, hN, g, eE, ve, eF, aVc, GR, cm, y, fT, iT, Jr, aiM, cj, dW, bAD, k, m, aDM, vF, eg, uL, qR, qm, bsh, cE, blB, QO, vE, yG, ht, fN, pk, blG, n, blD, aj, oY, fr, uE, aUX, bd, qN, qW, cd, ai, eh, ale, eW, aJf, aPb, ft, xC, adX, aMj, kH, h, dL, aaA, blO, aDC, ux, ey, he, blX, rk, nP, OV, mR, eZ, A, MD, aXy, aHW, sp, ff, lb, ge, fe, yq, aMJ, CQ, gG, kk, cM, Zj, lx, Wt, jq, blF, blH, abV, adZ, W, NL, dB, hk, aZL, lf, mg, JM, bm, uz, fl, aCD, DY, Pr, B, abW, it, bL, BlR, jv, fM, bll, eR, fG, jx, rf, aA, j, V, q, J, eA, o, ga, aWb, blC, blN, fh, qp, ld, yg, aT, yu, hK, rz, DX, ae, aZQ, blW, hM, bf, iU, Y, lC, r, afq, lo, aDm, en, t, pb, bN, lY, ck, ajl, Ta, OW, sn, c, ald, hA, ig, nM, iM, AH, iQ, Zg, CP, auT, Da, dy, gT, bt, Kd, aaT, aFe, Ke, aD, dM, iW, ha, jh, aHv, iD, dG, jE, blT, oL, hF, bCx, XB, ad, qC, dP, kL, hX, F, od, av, afm, blV, acu, Ot, ds, azq, qh, oW, akJ, hD, rN, vc, JK, lQ, bsn, yF, uQ, d, BN, ue, lU, je, KS, wc, azP, bHX, eH, dN, hv, yy, aF, hp, cr, Fq, Pd, iV, dr, aYt, LB, Bv, zr, axs, yE, blS, arE, Ny, bE, aED, Om, Lj, aCl, oQ, i, lC, ra, ne, sH, pC); #c1(HLA-B) c2(XP_011546344) c3(major histocompatibility complex, class I, B isoform X7) c4(345053) c5(345054, 345055, 345056, 345057, 345058, 345059, 345060, 345061, 345062, 345063, 345064, 345065, 345066, 345067, 345068, 345069) c6( )c7(bM, T, aC, xS, fR, fV, if, e, al, bO, CH, eb, fd, bwS, gA, qj, ci, bh, aoj, aY, Tz, aMB, bv, AW, wD, xy, vV, rw, ax, fS, bFU, dV, ja, aU, azm, rB, K, hu, wt, hH, LG, eL, bc, aiJ, cp, aa, hm, mH, aEV, bG, blP, aW, axu, asD, mQ, HA, LZ, blU, uY, hN, g, eE, ve, eF, aVc, GR, cm, y, fT, iT, Jr, aiM, cj, dW, bAD, k, m, aDM, vF, eg, uL, qR, qm, bsh, cE, blB, QO, vE, yG, ht, fN, pk, blG, n, blD, aj, oY, fr, uE, aUX, bd, qN, qW, cd, ai, eh, aJe, eW, aJf, aPb, ft, xC, adX, aMj, kH, h, dL, aaA, blO, aDC, ux, ey, he, blX, rk, nP, OV, mR, eZ, A, MD, aXy, aHW, sp, ff, lb, ge, fe, yq, aMJ, CQ, gG, kk, cM, Zj, lx, Wt, jq, blF, blH, abV, adZ, W, NL, dB, hk, aZL, lf, mg, JM, bm, uz, fl, aCD, DY, Pr, B, abW, it, bL, blR, jv, fM, bll, eR, fG, jx, rf, aA, j, V, q, J, eA, o, ga, aWb, blC, blN, fh, qp, ld, yg, aT, yu, hK, rz, DX, ae, aZQ, blW, hM, bf, iU, Y, lC, r, afq, lo, aDm, en, t, pb, bN, lY, ck, ajl, Ta, OW, sn, c, and, hA, ig, nM, iM, AH, iQ, Zg, CP, auT, Da, dy, gT, bt, Kd, aaT, aFe, Ke, aD, dM, iW, ha, jh, aHv, iD, dG, jE, blT, oL, hF, bCx, XB, ad, qC, dP, kL, hX, F, od, av, afm, blV, acu, Ot, ds, azq, qh, oW, akJ, hD, rN, vc, JK, lQ, bsn, yF, uQ, d, BN, ue, lU, je, KS, wc, azP, bHX, eH, dN, hv, yy, aF, hp, cr, Fq, Pd, iV, dr, aYt, LB, Bv, zr, axs, yE, blS, arE, Ny, bE, aED, Om, Lj, aCl, oQ, i, lC, ra, ne, sH, pC); #c1(HLA-B) c2(XP_011546532) c3(major histocompatibility complex, class I, B isoform X7) c4(345247, 345248, 345249, 345250) c5(345251, 345252, 345253, 345254, 345255, 345256, 345257, 345258, 345259, 345260, 345261, 345262) c6( )c7(bM, T, aC, xS, fR, fV, if, e, al, bO, CH, eb, fd, bwS, gA, qj, ci, bh, aoj, aY, Tz, aMB, bv, AW, wD, xy, vV, rw, aX, fS, bFU, dV, ja, aU, azm, rB, K, hu, wt, hH, LG, eL, bc, aiJ, cp, aa, hm, mH, aEV, bG, blP, aW, axu, asD, mQ, HA, LZ, blU, uY, hN, g, eE, ve, eF, aVc, GR, cm, y, fT, iT, Jr, aiM, cj, dW, bAD, k, m, aDM, vF, eg, uL, qR, qm, bsh, cE, blB, QO, vE, yG, ht, fN, pk, blG, n, blD, aj, oY, fr, uE, aUX, bd, qN, qW, cd, ai, eh, ale, eW, aJf, aPb, ft, xC, adX, aMj, kH, h, dL, aaA, blO, aDC, ux, ey, he, blX, rk, nP, OV, mR, eZ, A, MD, aXy, aHW, sp, ff, lb, ge, fe, yq, aMJ, CQ, gG, kk, cM, Zj, lx, Wt, jq, blF, blH, abV, adZ, W, NL, dB, hk, aZL, lf, mg, JM, bm, uz, fL, aCD, DY, Pr, B, abW, it, bL, blR, jv, fM, bll, eR, fG, jx, rf, aA, j, V, q, J, eA, o, ga, aWb, blC, blN, fh, qp, ld, yg, aT, yu, hK, rz, DX, ae, aZQ, blW, hM, bf, iU, Y, lC, r, afq, lo, aDm, en, t, pb, bN, lY, ck, ajl, Ta, OW, sn, c, aQd, hA, ig, nM, iM, AH, iQ, Zg, CP, auT, Da, dy, gT, bt, Kd, aaT, aFe, Ke, aD, dM, iW, ha, jh, aHv, iD, dG, JE, blT, oL, hF, bCx, XB, ad, qC, dP, kL, hX, F, od, av, afm, blV, acu, Ot, ds, azq, qh, oW, akJ, hD, rN, vc, JK, lQ, bsn, yF, uQ, d, BN, ue, lU, je, KS, wc, azP, bHX, eH, dN, hv, yy, aF, hp, cr, Fq, Pd, iV, dr, aYt, LB, Bv, zr, axs, yE, blS, arE, Ny, bE, aED, Om, Lj, aCl, oQ, i, lC, ra, ne, sH, pC); #c1(HLA-B) c2(XP_011546724) c3(major histocompatibility complex, class I, B isoform X7) c4(345484) c5(345485, 345486, 345487, 345488, 345489, 345490, 345491, 345492, 345493, 345494, 345495) c6( )c7(bM, T, aC, xS, fR, fV, if, e, al, bO, CH, eb, fd, bwS, gA, qj, ci, bh, aoj, aY, Tz, aMB, bv, AW, wD, xy, vV, rw, ax, fS, bFU, dV, ja, aU, azm, rB, K, hu, wt, hH, LG, eL, bc, aiJ, cp, aa, hm, mH, aEV, bG, blP, aW, axu, asD, mQ, HA, LZ, blU, uY, hN, g, eE, ve, eF, aVc, GR, cm, y, fT, iT, Jr, aiM, cj, dW, bAD, k, m, aDM, vF, eg, uL, qR, qm, bsh, cE, blB, QO, vE, yG, ht, fN, pk, blG, n, blD, aj, oY, fr, uE, aUX, bd, qN, qW, cd, ai, eh, ale, eW, aJf, aPb, ft, xC, adX, aMj, kH, h, dL, aaA, blO, aDC, ux, ey, he, blX, rk, nP, OV, mR, eZ, A, MD, aXy, aHW, sp, ff, lb, ge, fe, yq, aMJ, CQ, gG, kk, cM, Zj, lx, Wt, jq, blF, blH, abV, adZ, W, NL, dB, hk, aZL, lf, mg, JM, bm, uz, fL, aCD, DY, Pr, B, abW, it, bL, blR, jv, fM, bll, eR, fG, jx, rf, aA, j, V, q, J, eA, o, ga, aWb, blC, blN, fh, qp, ld, yg, aT, yu, hK, rz, DX, ae, aZQ, blW, hM, bf, iU, Y, lC, r, afq, lo, aDm, en, t, pb, bN, lY, ck, ajl, Ta, OW, sn, c, aQd, hA, ig, nM, iM, AH, iQ, Zg, CP, auT, Da, dy, gT, bt, Kd, aaT, aFe, Ke, aD, dM, iW, ha, jh, aHv, iD, dG, jE, blT, oL, hF, bCx, XB, ad, qC, dP, kL, hX, F, od, av, afm, blV, acu, Ot, ds, azq, qh, oW, akJ, hD, rN, vc, JK, lQ, bsn, yF, uQ, d, BN, ue, lU, je, KS, wc, azP, bHX, eH, dN, hv, yy, aF, hp, cr, Fq, Pd, iV, dr, aYt, LB, Bv, zr, axs, yE, blS, arE, Ny, bE, aED, Om, Lj, aCl, oQ, i, lC, ra, ne, sH, pC); #c1(HLA-C) c2(NP_001229971) c3(HLA class I histocompatibility antigen, Cw-1 alpha chain precursor) c4(85540, 85541, 85542, 85543, 85544, 85545, 85546, 85547, 85548, 85549, 85550, 85551, 85552, 85553, 85554, 85555, 85556, 85557, 85558, 85559, 85560) c5(85561, 85562, 85563, 85564, 85565, 85566, 85567, 85568, 85569, 85570, 85571, 85572, 85573, 85574, 85575, 85576, 85577, 85578, 85579, 85580, 85581, 85582, 85583, 85584, 85585, 85586, 85587, 85588, 85589, 85590, 85591, 85592, 85593, 85594, 85595, 85596, 85597, 85598, 85599, 85600, 85601, 85602, 85603, 85604, 85605, 85606, 85607, 85608, 85609, 85610, 85611, 85612, 85613, 85614, 85615, 85616, 85617, 85618, 85619, 85620, 85621, 85622, 85623, 85624, 85625, 85626, 85627, 85628, 85629, 85630, 85631, 85632, 85633, 85634, 85635, 85636, 85637, 85638, 85639, 85640, 85641, 85642, 85643, 85644, 85645, 85646, 85647, 85648, 85649, 85650, 85651, 85652, 85653, 85654, 85655, 85656, 85657, 85658, 85659, 85660, 85661, 85662, 85663, 85664, 85665, 85666, 85667, 85668, 85669, 85670, 85671, 85672, 85673, 85674, 85675, 85676, 85677, 85678, 85679, 85680, 85681, 85682, 85683, 85684, 85685, 85686, 85687, 85688, 85689, 85690, 85691, 85692, 85693, 85694, 85695, 85696, 85697, 85698, 85699, 85700, 85701, 85702, 85703, 85704, 85705, 85706, 85707, 85708, 85709, 85710, 85711, 85712, 85713, 85714, 85715, 85716, 85717, 85718, 85719, 85720, 85721, 85722, 85723, 85724, 85725, 85726, 85727, 85728, 85729, 85730, 85731, 85732, 85733, 85734, 85735, 85736, 85737, 85738, 85739, 85740, 85741, 85742, 85743, 85744, 85745, 85746, 85747, 85748, 85749, 85750, 85751, 85752, 85753, 85754, 85755) c6( )c7(bM, T, aC, xS, fR, fV, if, al, bO, eb, fd, gA, qj, ci, bh, aMB, Vp, AW, fS, bFU, blY, azm, rB, hH, mH, axu, mQ, hl, HA, da, hN, ve, uC, cm, GR, bw, iP, y, fT, iT, Jr, nB, qr, bAD, k, pw, eg, qm, avY, aFQ, ht, fN, n, aj, fr, qN, qW, eW, aMp, Bj, h, he, blX, nP, A, mR, bJc, byn, lb, ge, fe, aq, cM, Zj, aBs, jq, blF, blH, dB, bJa, hk, JM, uz, fL, aCD, lB, B, bL, blR, jv, fM, eR, aZy, jx, aA, v, j, V, eA, J, ga, blC, mP, blN, ld, aT, bJb, blW, aFS, hM, iU, Y, r, afq, lo, en, t, pb, vk, bN, ck, OW, sn, ua, nM, iQ, Zg, CP, AH, bJd, Da, byK, bt, Kd, aaT, aD, iW, jh, jE, oL, hF, mC, wD, hX, dP, hd, F, od, Db, qh, hD, rN, JK, lQ, uQ, d, lU, aus, ff, azP, cr, eZ, hv, aF, hp, Pd, iV, blZ, bF, D, gk, yE, aED, aCl, oQ, i, lf, fh, ne, rk, pC); #c1 (HLA-C) c2(NP_002108) c3(HLA class I histocompatibility antigen, Cw-l alpha chain precursor) c4(124685, 124686, 124687, 124688, 124689, 124690, 124691, 124692, 124693, 124694, 124695) c5(124696, 124697, 124698, 124699, 124700, 124701, 124702, 124703, 124704, 124705, 124706, 124707, 124708, 124709, 124710, 124711, 124712, 124713, 124714, 124715, 124716, 124717, 124718, 124719, 124720, 124721, 124722, 124723, 124724, 124725, 124726, 124727, 124728, 124729, 124730, 124731, 124732, 124733, 124734, 124735, 124736, 124737, 124738, 124739, 124740, 124741, 124742, 124743, 124744, 124745, 124746, 124747, 124748, 124749, 124750, 124751, 124752, 124753, 124754, 124755, 124756, 124757, 124758, 124759, 124760, 124761) c6( )c7(bM, T, aC, xS, fR, fV, if, al, bO, eb, fd, gA, qj, ci, bh, aMB, Vp, AW, fS, bFU, blY, azm, rB, hH, mH, axu, mQ, hl, HA, da, hN, ve, uC, cm, GR, bw, iP, y, fT, iT, Jr, nB, qr, bAD, k, pw, eg, qm, avY, aFQ, ht, fN, n, aj, fr, qN, qW, eW, aMp, Bj, h, he, blX, nP, A, mR, bJc, byn, lb, ge, fe, aq, cM, Zj, aBs, jq, blF, blH, dB, bJa, hk, JM, uz, fL, aCD, lB, B, bL, blR, jv, fM, eR, aZy, jx, aA, v, j, V, eA, J, ga, blC, mP, blN, ld, aT, bJb, blW, aFS, hM, iU, Y, r, afq, lo, en, t, pb, vk, bN, ck, OW, sn, ua, nM, iQ, Zg, CP, AH, bJd, Da, byK, bt, Kd, aaT, aD, iW, jh, jE, oL, hF, mC, wD, hX, dP, hd, F, od, Db, qh, hD, rN, JK, lQ, ul, d, lU, aus, ff, azP, cr, eZ, hv, aF, hp, Pd, iV, blZ, bF, D, gk, yE, aED, aCl, oQ, i, lf, fh, ne, rk, pC); #c1(HLA-DMA) c2(NP_006111) c3(HLA class II histocompatibility antigen, DM alpha chain precursor) c4(161948, 161949, 161950, 161951, 161952, 161953, 161954, 161955, 161956, 161957, 161958, 161959) c5(161960, 161961, 161962, 161963, 161964, 161965, 161966, 161967, 161968, 161969, 161970, 161971, 161972, 161973, 161974, 161975, 161976, 161977, 161978, 161979, 161980, 161981, 161982, 161983, 161984, 161985, 161986, 161987, 161988, 161989, 161990, 161991, 161992, 161993, 161994, 161995, 161996, 161997, 161998, 161999, 162000, 162001, 162002, 162003, 162004, 162005, 162006, 162007, 162008, 162009, 162010, 162011, 162012, 162013, 162014, 162015, 162016, 162017, 162018, 162019, 162020, 162021, 162022, 162023, 162024, 162025, 162026, 162027, 162028, 162029, 162030, 162031, 162032, 162033, 162034, 162035, 162036, 162037, 162038, 162039, 162040, 162041, 162042, 162043, 162044, 162045, 162046, 162047, 162048, 162049, 162050, 162051, 162052, 162053, 162054, 162055, 162056, 162057, 162058, 162059, 162060, 162061, 162062, 162063, 162064, 162065, 162066, 162067, 162068, 162069, 162070, 162071, 162072, 162073, 162074, 162075, 162076, 162077, 162078, 162079, 162080, 162081, 162082, 162083, 162084, 162085, 162086, 162087, 162088, 162089, 162090, 162091, 162092, 162093, 162094, 162095, 162096, 162097, 162098, 162099, 162100, 162101, 162102, 162103, 162104, 162105, 162106, 162107, 162108, 162109, 162110, 162111, 162112, 162113, 162114, 162115, 162116, 162117, 162118, 162119, 162120, 162121, 162122) c6( )c7(bL, T, id, al, ez, aj, dG, bJe, V, eA, J, bv, dm, h, ey, uQ, OV, akV, Sn, cr, eZ, pL, pb, gk, iQ, vZ, mf, fl, y); #c1(HLA-DMB) c2(NP_002109) c3(HLA class II histocompatibility antigen, DM beta chain precursor) c4(124762, 124763, 124764, 124765, 124766, 124767, 124768, 124769, 124770, 124771, 124772, 124773, 124774, 124775) c5(124776, 124777, 12478, 124779, 124780, 124781, 124782, 124783, 124784, 124785, 124786, 124787, 124788, 124789, 124790, 124791, 124792, 124793, 124794, 124795, 124796, 124797, 124798, 124799, 124800, 124801, 124802, 124803, 124804, 124805, 124806, 124807, 124808, 124809, 124810, 124811, 124812, 124813, 124814, 124815, 124816, 124817, 124818, 124819, 124820, 124821, 124822, 124823, 124824, 124825, 124826, 124827, 124828, 124829, 124830, 124831, 124832, 124833, 124834, 124835, 124836, 124837, 124838, 124839, 124840, 124841, 124842, 124843, 124844, 124845, 124846, 124847, 124848, 124849, 124850, 124851, 124852, 124853, 124854, 124855, 124856, 124857, 124858, 124859, 124860, 124861, 124862, 124863, 124864, 124865, 124866, 124867, 124868, 124869, 124870, 124871, 124872, 124873, 124874, 124875, 124876, 124877, 124878, 124879, 124880, 124881, 124882, 124883, 124884, 124885, 124886, 124887, 124888, 124889, 124890, 124891, 124892, 124893, 124894, 124895, 124896, 124897, 124898, 124899, 124900, 124901, 124902, 124903, 124904, 124905, 124906, 124907, 124908, 124909, 124910, 124911, 124912, 124913, 124914, 124915, 124916, 124917, 124918, 124919, 124920, 124921, 124922, 124923, 124924, 124925, 124926, 124927, 124928, 124929, 124930, 124931, 124932, 124933, 124934, 124935, 124936, 124937, 124938, 124939, 124940, 124941, 124942, 124943, 124944, 124945, 124946, 124947, 124948, 124949, 124950, 124951, 124952, 124953, 124954, 124955, 124956, 124957, 124958, 124959, 124960, 124961, 124962, 124963, 124964, 124965, 124966, 124967, 124968, 124969, 124970, 124971, 124972, 124973, 124974, 124975, 124976, 124977, 124978, 124979, 124980, 124981, 124982, 124983, 124984, 124985, 124986, 124987, 124988, 124989, 124990, 124991, 124992, 124993, 124994, 124995, 124996, 124997, 124998, 124999, 125000, 125001, 125002, 125003, 125004, 125005, 125006, 125007, 125008, 125009, 125010, 125011) c6( )c7(bL, ez, aj, dG, bJe, V, J, dm, h, ey, lM, uQ, OV, akV, A, cr, eZ, pL, t, pb, vZ, mf, fL, y); #c1(HLA-DOA) c2(NP_002110) c3(HLA class II histocompatibility antigen, DO alpha chain precursor) c4(125012) c5(125013, 125014, 125015, 125016, 125017, 125018, 125019, 125020, 125021, 125022, 125023, 125024, 125025, 125026, 125027, 125028, 125029, 125030, 125031, 125032, 125033, 125034, 125035, 125036, 125037, 125038, 125039, 125040, 125041) c6( )c7(bM, T, if, yG, aA, fd, n, aj, qj, bh, qN, fr, V, J, bCx, hX, xy, aX, yg, adX, aMj, kH, aT, dL, hD, DY, LG, eL, bc, yF, hm, hM, A, mR, bf, CN, aW, sp, Y, hl, cr, eZ, aF, hp, aMJ, eE, t, adZ, bE, arE, cj, m, y, bt, pC); #c1(HLA-DOA) c2(XP_005249104) c3(HLA class II histocompatibility antigen, DO alpha chain isoform X1) c4(268998, 268999) c5(269000, 269001, 269002, 269003, 269004, 269005, 269006, 269007, 269008, 269009, 269010, 269011, 269012, 269013, 269014, 269015, 269016, 269017, 269018, 269019, 269020, 269021, 269022, 269023, 269024, 269025, 269026, 269027, 269028) c6( )c7(bM, T, if, yG, aA, fd, n, aj, qj, bh, qN, fr, V, J, bCx, hX, xy, aX, yg, adX, aMj, kH, aT, dL, hD, DY, LG, eL, bc, yF, hm, hM, A, mR, bf, CN, aW, sp, Y, hl, cr, eZ, af, hp, aMJ, eE, t, adZ, bE, arE, cj, m, y, bt, pC); #c1(HLA-DOA) c2(XP_005275163) c3(HLA class II histocompatibility antigen, DO alpha chain isoform X1) c4(285574) c5( )c6( )c7(bM, T, if, yG, aA, fd, n, aj, qj, bh, qN, fr, V, J, bCx, hX, xy, aX, yg, adX, aMj, kH, aT, dL, hD, DY, LG, eL, bc, yF, hm, hM, A, mR, bf, CN, aW, sp, Y, hl, cr, eZ, aF, hp, aMJ, eE, t, adZ, bE, arE, cj, m, y, bt, pC); #c1 (HLA-DOA) c2(XP_006715139) c3(HLA class II histocompatibility antigen, DO alpha chain isoform X2) c4( )c5(288764, 288765, 288766, 288767, 288768, 288769, 288770) c6( )c7(bM, T, if, yG, aA, fd, n, aj, qj, bh, qN, fr, V, J, bCx, hX, xy, aX, yg, adX, aMj, kH, aT, dL, hD, DY, LG, eL, bc, yF, hm, hM, A, mR, bf, CN, aW, sp, Y, hl, cr, eZ, af, hp, aMJ, eE, t, adZ, bE, arE, cj, m, y, bt, pC); #c1(HLA-DOA) c2(XP_011512860) c3(HLA class II histocompatibility antigen, DO alpha chain isoform X3) c4(304008) c5(304009, 304010, 304011, 304012, 304013, 304014, 304015, 304016, 304017, 304018, 304019, 304020, 304021, 304022, 304023, 304024, 304025, 304026, 304027, 304028, 304029, 304030, 304031, 304032, 304033, 304034, 304035, 304036, 304037, 304038, 304039, 304040, 304041, 304042, 304043, 304044, 304045, 304046, 304047, 304048, 304049, 304050, 304051, 304052, 304053, 304054, 304055, 304056) c6( )c7(bM, T, if, yG, aA, fd, n, aj, qj, bh, qN, fr, V, J, bCx, hX, xy, aX, yg, adX, aMj, kH, aT, dL, hD, DY, LG, eL, bc, yF, hm, hM, A, mR, bf, CN, aW, sp, Y, hl, cr, eZ, aF, hp, aMJ, eE, t, adZ, bE, art, cj, m, y, bt, pC); #c1 (HLA-DOB) c2(NP_002111) c3(HLA class II histocompatibility antigen, DO beta chain precursor) c4(125042, 125043, 125044) c5(125045, 125046, 125047, 125048, 125049, 125050, 125051, 125052, 125053, 125054, 125055, 125056, 125057, 125058, 125059, 125060, 125061, 125062, 125063, 125064, 125065, 125066, 125067, 125068, 125069, 125070, 125071, 125072, 125073, 125074, 125075, 125076, 125077, 125078, 125079, 125080, 125081, 125082, 125083, 125084, 125085, 125086, 125087, 125088, 125089, 125090, 125091, 125092, 125093, 125094) c6( )c7(AW, A, aW, eZ, cr, aT, fd, hH); #c1(HLA-DPA1) c2(NP_001229454) c3(HLA class II histocompatibility antigen, DP alpha 1 chain precursor) c4(84442, 84443, 84444, 84445) c5(84446, 84447, 84448, 84449, 84450, 84451, 84452, 84453, 84454, 84455, 84456, 84457, 84458, 84459, 84460, 84461, 84462, 84463, 84464, 84465, 84466, 84467, 84468, 84469, 84470, 84471, 84472, 84473, 84474, 84475, 84476, 84477, 84478, 84479, 84480, 84481, 84482, 84483, 84484, 84485, 84486, 84487, 84488, 84489, 84490, 84491, 84492, 84493, 84494, 84495, 84496, 84497, 84498, 84499, 84500, 84501, 84502, 84503, 84504, 84505, 84506, 84507, 84508, 84509, 84510, 84511, 84512, 84513, 84514, 84515, 84516, 84517, 84518, 84519, 84520, 84521, 84522, 84523, 84524, 84525, 84526, 84527, 84528, 84529, 84530, 84531, 84532, 84533, 84534, 84535, 84536, 84537, 84538, 84539, 84540, 84541, 84542, 84543, 84544, 84545, 84546, 84547, 84548, 84549, 84550, 84551, 84552, 84553, 84554, 84555, 84556) c6( )c7(bL, bM, cL, jE, ci, ai, hX, ady, hd, cc, akJ, h, sX, yF, A, mR, Y, eZ, CQ, hJ, uC, pb, uy, bAD, dy, Xa, WX); #c1(HLA-DPA1) c2(XP_011512861) c3(HLA class II histocompatibility antigen, DP alpha 1 chain isoform X1) c4(304057) c5(304058, 304059, 304060, 304061, 304062, 304063, 304064, 304065, 304066, 304067, 304068, 304069, 304070, 304071, 304072, 304073, 304074, 304075, 304076, 304077, 304078, 304079, 304080, 304081, 304082, 304083, 304084, 304085, 304086, 304087, 304088, 304089, 304090, 304091, 304092, 304093, 304094, 304095, 304096, 304097, 304098, 304099, 304100, 304101, 304102, 304103, 304104, 304105, 304106, 304107, 304108, 304109, 304110, 304111, 304/12, 304113) c6( )c7(bL, bM, cL, jE, ci, ai, hX, ady, hd, cc, akJ, h, sX, yF, A, mR, Y, eZ, CQ, hJ, uC, pb, uy, bAD, dy, Xa, WX); #c1(HLA-DPB1) c2(NP_002112) c3(HLA class II histocompatibility antigen, DP beta 1 chain precursor) c4(125095, 125096, 125097, 125098, 125099, 125100, 125101, 125102, 125103, 125104, 125105, 125106, 125107, 125108, 125109, 125110, 125111, 125112, 125113, 125114, 125115, 125116, 125117, 125118, 125119, 125120, 125121, 125122) c5(125123, 125124, 125125, 125126, 125127, 125128, 125129, 125130, 125131, 125132, 125133, 125134, 125135, 125136, 125137, 125138, 125139, 125140, 125141, 125142, 125143, 125144, 125145, 125146, 125147, 125148, 125149, 125150, 125151, 125152, 125153, 125154, 125155, 125156, 125157, 125158, 125159, 125160, 125161, 125162, 125163, 125164, 125165, 125166, 125167, 125168, 125169, 125170, 125171, 125172, 125173, 125174, 125175, 125176, 125177, 125178, 125179, 125180, 125181, 125182, 125183, 125184, 125185, 125186, 125187, 125188, 125189, 125190, 125191, 125192, 125193, 125194, 125195, 125196, 125197, 125198, 125199, 125200, 125201, 125202, 125203, 125204, 125205, 125206, 125207, 125208, 125209, 125210, 125211, 125212, 125213, 125214, 125215, 125216, 125217, 125218, 125219, 125220, 125221, 125222, 125223, 125224, 125225, 125226, 125227, 125228, 125229, 125230, 125231, 125232, 125233, 125234, 125235, 125236, 125237, 125238, 125239, 125240, 125241, 125242, 125243, 125244, 125245, 125246, 125247, 125248, 125249, 125250, 125251, 125252, 125253, 125254, 125255, 125256, 125257, 125258, 125259, 125260, 125261, 125262, 125263, 125264, 125265, 125266, 125267, 125268, 125269, 125270, 125271, 125272, 125273, 125274, 125275, 125276, 125277, 125278, 125279, 125280, 125281, 125282, 125283, 125284, 125285, 125286, 125287, 125288, 125289, 125290, 125291, 125292, 125293, 125294, 125295, 125296, 125297, 125298, 125299, 125300, 125301, 125302, 125303, 125304, 125305, 125306, 125307, 125308, 125309, 125310, 125311, 125312, 125313, 125314, 125315, 125316, 125317, 125318, 125319, 125320, 125321, 125322, 125323, 125324, 125325, 125326, 125327, 125328, 125329, 125330, 125331, 125332, 125333) c6( )c7(DS, bM, T, asg, adP, if, al, fd, cL, lR, bJh, rm, ci, bh, NF, x, vV, JD, bW, ja, hH, eL, bc, azh, aa, axu, mQ, hl, pL, ve, bw, aVc, GR, ou, y, iT, Jr, uy, vy, cj, bAD, k, pw, eg, P, bzb, ez, blG, aj, qN, ai, br, tD, bJf, cc, vM, h, eV, dL, ey, bEg, mJ, nP, A, mR, fe, CQ, mo, kk, cM, jq, aZL, lf, uz, eo, Xa, abW, B, fG, jx, ex, bJg, V, pd, J, o, MK, ady, aT, sX, to, hM, s, iU, xR, Y, aDm, hf, pb, ck, ac, kB, ig, lV, Zg, AH, aGY, hr, Da, Tu, bt, blu, aDy, jh, bs, iD, jE, hF, hX, dP, ot, od, oz, qh, LA, hD, vc, yF, uQ, d, ue, cr, eZ, hp, Pd, iV, qa, aYt, gk, Ny, aDg, rH, i, lC, WX, rk); #c1(HLA-DPB1) c2(XP_006715141) c3(HLA class II histocompatibility antigen, DP beta 1 chain isoform X1) c4( )c5(288771, 288772, 288773, 288774, 288775, 288776, 288777, 288778, 288779, 288780, 288781, 288782, 288783, 288784, 288785, 288786, 288787, 288788, 288789, 288790, 288791, 288792) c6( )c7(DS, bM, T, asg, adP, if, al, fd, cL, lR, bJh, rm, ci, bh, NF, x, vV, JD, bW, ja, hH, eL, bc, azh, aa, axu, mQ, hl, pL, ve, bw, aVc, GR, ou, y, iT, Jr, uy, vy, cj, bAD, k, pw, eg, P, bzb, ez, blG, aj, qN, ai, br, tD, bJf, cc, vM, h, eV, dL, ey, bEg, mJ, nP, A, mR, fe, CQ, mo, kk, cM, jq, aZL, lf, uz, eo, Xa, abW, B, fG, jx, eX, bJg, V, pd, J, o, MK, ady, aT, sX, to, hM, s, iU, xR, Y, aDm, hf, pb, ck, ac, KB, ig, lV, Zg, AH, aGY, hr, Da, Tu, bt, blu, aDy, jh, bs, iD, jE, hF, hX, dP, ot, od, oz, qh, LA, hD, vc, yF, uQ, d, ue, cr, eZ, hp, Pd, iV, qa, aYt, gk, Ny, aDg, rH, i, lC, WX, rk); #c1(HLA-DPB1) c2(XP_006725103) c3(HLA class II histocompatibility antigen, DP beta 1 chain isoform X1) c4(296564) c5(296565, 296566, 296567, 296568, 296569, 296570, 296571, 296572) c6( )c7(DS, bM, T, asg, adP, if, al, fd, cL, lR, bJh, rm, ci, bh, NF, x, vV, JD, bW, ja, hH, eL, bc, azh, aa, axu, mQ, hl, pL, ve, bw, aVc, GR, ou, y, iT, Jr, uy, vy, cj, bAD, k, pw, eg, P, bzb, ez, blG, aj, qN, ai, br, tD, bJf, cc, vM, h, eV, dL, ey, bEg, mJ, nP, A, mR, fe, CQ, mo, kk, cM, jq, aZL, lf, uz, eo, Xa, abW, B, fG, jx, ex, bJg, V, pd, J, o, MK, ady, aT, sX, to, hM, s, iU, xR, Y, aDm, hf, pb, ck, ac, kB, ig, lV, Zg, AH, aGY, hr, Da, Tu, bt, blu, aDy, jh, bs, iD, jE, hF, hX, dP, ot, od, oz, qh, LA, hD, vc, yF, uQ, d, ue, cr, eZ, hp, Pd, iV, qa, aYt, gk, Ny, aDg, H, i, lC, WX, rk); #c1 (HLA-DQA1) c2(NP_002113) c3(HLA class II histocompatibility antigen, DQ alpha 1 chain precursor) c4(125334, 125335) c5(125336, 125337, 125338, 125339, 125340, 125341, 125342, 125343, 125344, 125345, 125346, 125347, 125348, 125349, 125350, 125351, 125352, 125353, 125354, 125355, 125356, 125357, 125358, 125359, 125360, 125361, 125362, 125363, 125364) c6( )c7(bM, fR, al, aeu, cL, qj, ci, bh, lR, Jt, AW, WX, JD, fS, ja, wt, bc, bn, Jl, aW, axu, lh, az, hl, mQ, HA, kt, hN, g, eE, ve, uC, fT, yl, iT, uy, mx, vy, dW, bAD, pw, lS, eg, blB, dD, wu, Hm, aw, yG, fN, ez, blG, aj, qN, aYv, qW, ai, xC, cc, h, nP, OV, mR, A, lb, bJi, ge, Ni, aq, fe, CQ, mo, cM, adz, jq, ar, lX, Qu, mg, JM, bm, te, awQ, eo, fL, aDE, Xa, it, Wl, abW, B, bL, jv, eR, dk, jx, aA, GP, cz, OY, wy, V, pd, J, o, ady, cF, aEE, fh, jZ, sl, bza, qp, aT, jj, an, hK, kU, lM, sX, aFS, blJ, hM, Y, lC, r, afq, en, t, pb, lY, ck, OW, sn, ig, lV, Da, dy, aDj, ao, yv, gV, dM, axd, jh, eq, jE, aGW, wD, hX, dP, hd, F, afm, azq, akJ, hD, vB, wm, uD, vO, yF, uQ, uX, WZ, bHo, ff, wc, bps, cr, eZ, dN, aF, hp, iV, bz, es, Bv, yE, aED, Lb, qu, XO, oQ, y, kS, iu, rk, pC); #c1(HLA-DQA1) c2(XP_003846516) c3(HLA class II histocompatibility antigen, DQ alpha 1 chain isoform X4) c4(266124) c5(266125, 266126, 266127, 266128, 266129, 266130, 266131, 266132, 266133, 266134, 266135, 266136, 266137, 266138, 266139, 266140, 266141, 266142) c6( )c7(bM, fR, al, aeu, cL, qj, ci, bh, lR, Jt, AW, WX, JD, fS, ja, wt, bc, bn, Jl, aW, axu, lh, az, hl, mQ, HA, kt, hN, g, eE, ve, uC, fT, yl, iT, uy, mx, vy, dW, bAD, pw, lS, eg, blB, dD, wu, Hm, aw, yG, fN, ez, blG, aj, qN, aYv, qW, ai, xC, cc, h, nP, OV, mR, A, lb, bJi, ge, Ni, aq, fe, CQ, mo, cM, adz, jq, ar, lX, Qu, mg, JM, bm, te, awQ, eo, fL, aDE, Xa, it, Wl, abW, B, bL, jv, eR, dk, jx, aA, GP, cz, OY, wy, V, pd, J, o, ady, cF, aEE, fh, jZ, sl, bza, qp, aT, jj, an, hK, kU, lM, sX, aFS, blJ, hM, Y, lC, r, afq, en, t, pb, lY, ck, OW, sn, ig, lV, Da, dy, aDj, ao, yv, gV, dM, axd, jh, eq, jE, aGW, wD, hX, dP, hd, F, afm, azq, akJ, hD, vB, wm, uD, vO, yF, ul, ux, WZ, bHo, ff, wc, bps, cr, eZ, dN, aF, hp, iV, bz, es, Bv, yE, aED, Lb, qu, XO, oQ, y, kS, iu, rk, pC); #c1(HLA-DQA1) c2(XP_005275165) c3(HLA class II histocompatibility antigen, DQ alpha 1 chain isoform X4) c4(285575) c5(285576, 285577, 285578, 285579, 285580, 285581, 285582, 285583, 285584, 285585, 285586, 285587, 285588, 285589, 285590, 285591, 285592, 285593, 285594, 285595, 285596, 285597, 285598, 285599, 285600, 285601, 285602, 285603, 285604, 285605) c6( )c7(bM, fR, al, aeu, cL, qj, ci, bh, lR, Jt, AW, WX, JD, fS, ja, wt, bc, bn, Jl, aW, axu, lh, az, hl, mQ, HA, kt, hN, g, eE, ve, uC, fT, yl, iT, uy, mx, vy, dW, bAD, pw, lS, eg, blB, dD, wu, Hm, aw, yG, fN, ez, blG, aj, qN, aYv, qW, ai, xC, cc, h, nP, OV, mR, A, lb, bJi, ge, Ni, aq, fe, CQ, mo, cM, adz, jq, ar, lX, Qu, mg, JM, bm, te, awQ, eo, fL, aDE, Xa, it, Wl, abW, B, bL, jv, eR, dk, jx, aA, GP, cz, OY, wy, V, pd, J, o, ady, cF, aEE, fh, jZ, sl, bza, qp, aT, jj, an, hK, kU, lM, sX, aFS, blJ, hM, Y, lC, r, afq, en, t, pb, lY, ck, OW, sn, ig, lV, Da, dy, aDj, ao, yv, gV, dM, axd, jh, eq, jE, aGW, wD, hX, dP, hd, F, afm, azq, akJ, hD, vB, wm, uD, vO, yF, uQ, ux, WZ, bHo, ff, wc, bps, cr, eZ, dN, aF, hp, iV, bz, es, Bv, yE, aED, Lb, qu, XO, oQ, y, kS, iu, rk, pC); #c1(HLA-DQA1) c2(XP_005275599) c3(HLA class II histocompatibility antigen, DQ alpha 1 chain isoform X4) c4(285649, 285650, 285651, 285652, 285653) c5(285654, 285655, 285656, 285657, 285658, 285659, 285660, 285661, 285662, 285663, 285664, 285665, 285666, 285667, 285668, 285669, 285670, 285671, 285672, 285673, 285674, 285675, 285676, 285677, 285678, 285679, 285680, 285681, 285682, 285683, 285684, 285685, 285686, 285687) c6( )c7(bM, fR, al, aeu, cL, qj, ci, bh, lR, Jt, AW, WX, JD, fS, ja, wt, bc, bn, Jl, aW, axu, lh, az, hl, mQ, HA, kt, hN, g, eE, ve, uC, fT, yl, iT, uy, mx, vy, dW, bAD, pw, lS, eg, blB, dD, wu, Hm, aw, yG, fN, ez, blG, aj, qN, aYv, qW, ai, xC, cc, h, nP, OV, mR, A, lb, bJi, ge, Ni, aq, fe, CQ, mo, cM, adz, jq, ar, lX, Qu, mg, JM, bm, te, awQ, eo, fl, aDE, Xa, it, Wl, abW, B, bL, jv, eR, dk, jx, aA, GP, cz, OY, wy, V, pd, J, o, ady, cF, aEE, fh, jZ, sl, bza, qp, aT, jj, an, hK, kU, lM, sX, aFS, blJ, hM, Y, lC, r, afq, en, t, pb, lY, ck, OW, sn, ig, lV, Da, dy, aDj, ao, yv, gV, dM, axd, jh, eq, jE, aGW, wD, hX, dP, hd, F, afm, azq, akJ, hD, vB, wm, uD, vO, yF, uQ, uX, WZ, bHo, ff, wc, bps, cr, eZ, dN, aF, hp, iV, bz, es, Bv, yE, aED, Lb, qu, XO, oQ, y, kS, iu, rk, pC); #c1 (HLA-DQA1) c2(XP_006715142) c3(HLA class II histocompatibility antigen, DQ alpha 1 chain isoform X1) c4(288793) c5(288794, 288795, 288796, 288797, 288798, 288799) c6( )c7(bM, fR, al, aeu, cL, qj, ci, bh, lR, Jt, AW, WX, JD, fS, ja, wt, bc, bn, Jl, aW, axu, lh, az, hl, mQ, HA, kt, hN, g, eE, ve, uC, fT, yl, iT, uy, mx, vy, dW, bAD, pw, lS, eg, blB, dD, wu, Hm, aw, yG, fN, ez, blG, aj, qN, aYv, qW, ai, xC, cc, h, nP, OV, mR, A, lb, bJi, ge, Ni, aq, fe, CQ, mo, cM, adz, jq, ar, lX, Qu, mg, JM, bm, te, awQ, eo, fL, aDE, Xa, it, Wl, abW, B, bL, jv, eR, dk, jx, aA, GP, cz, OY, wy, V, pd, J, o, ady, cf, aEE, fh, jZ, sl, bza, qp, aT, jj, an, hK, kU, lM, sX, aFS, blJ, hM, Y, lC, r, afq, en, t, pb, lY, ck, OW, sn, ig, lV, Da, dy, aDj, ao, yv, gV, dM, axd, jh, eq, jE, aGW, wD, hX, dP, hd, F, afm, azq, akJ, HD, vB, wm, uD, vO, yF, uQ, ux, WZ, bHo, ff, wc, bps, cr, eZ, dN, aF, hp, iV, bz, es, Bv, yE, aED, Lb, qu, XO, oQ, y, kS, iu, rk, pC); #c1(HLA-DQA1) c2(XP_006725547) c3(HLA class II histocompatibility antigen, DQ alpha 1 chain isoform X4) c4(296835, 296836, 296837, 296838) c5(296839, 296840, 296841, 296842, 296843, 296844, 296845, 296846, 296847, 296848, 296849, 296850, 296851, 296852, 296853, 296854, 296855, 296856, 296857, 296858, 296859, 296860, 296861, 296862, 296863, 296864, 296865, 296866, 296867, 296868, 296869, 296870, 296871, 296872, 296873, 296874, 296875, 296876, 296877, 296878, 296879, 296880, 296881, 296882, 296883) c6( )c7(bM, fR, al, aeu, cL, qj, ci, bh, lR, Jt, AW, WX, JD, fS, ja, wt, bc, bn, Jl, aW, axu, lh, az, hl, mQ, HA, kt, hN, g, eE, ve, uC, fT, yl, iT, uy, mx, vy, dW, bAD, pw, lS, eg, blB, dD, wu, Hm, aw, yG, fN, ez, blG, aj, qN, aYv, qW, ai, xC, cc, h, nP, OV, mR, A, lb, bJi, ge, Ni, aq, fe, CQ, mo, cM, adz, jq, ar, lX, Qu, mg, JM, bm, te, awQ, eo, fL, aDE, Xa, it, Wl, abW, B, bL, jv, eR, dk, jx, aA, GP, cz, DY, wy, V, pd, J, o, ady, cF, aEE, fh, jZ, sl, bza, qp, aT, jj, an, hK, kU, lM, sX, aFS, blJ, hM, Y, lC, r, afq, en, t, pb, lY, ck, OW, sn, ig, lV, Da, dy, aDj, ao, yv, gV, dM, axd, jh, eq, jE, aGW, wD, hX, dP, hd, F, afm, azq, akJ, hD, vB, wm, uD, vO, yF, uQ, ux, WZ, bHo, ff, wc, bps, cr, eZ, dN, aF, hp, iV, bz, es, Bv, yE, aED, Lb, qu, XO, oQ, y, kS, iu, rk, pC); #c1 (HLA-DQA1) c2(XP_011546178) c3(HLA class II histocompatibility antigen, DQ alpha 1 chain isoform X3) c4(344883, 344884) c5(344885, 344886, 344887, 344888, 344889, 344890, 344891, 344892, 344893, 344894, 344895, 344896, 344897, 344898, 344899, 344900, 344901) c6( )c7(bM, fR, al, aeu, cL, qj, ci, bh, lR, Jt, AW, WX, JD, fS, ja, wt, bc, bn, Jl, aW, axu, lh, az, hl, mQ, HA, kt, hN, g, eE, ve, uC, fT, yl, iT, uy, mx, vy, dW, bAD, pw, lS, eg, blB, dD, wu, Hm, aw, yG, fN, ez, blG, aj, qN, aYv, qW, ai, xC, cc, h, nP, OV, mR, A, lb, bJi, ge, Ni, aq, fe, CQ, mo, cM, adz, jq, ar, lX, Qu, mg, JM, bm, te, awQ, eo, fL, aDE, Xa, it, Wl, abW, B, bL, jv, eR, dK, jx, aA, GP, cz, OY, wy, V, pd, J, o, ady, cF, aEE, fh, jZ, sl, bza, qp, aT, jj, an, hK, kU, lM, sX, aFS, blJ, hM, Y, lC, r, afq, en, t, pb, lY, ck, OW, sn, ig, lV, Da, dy, aDj, ao, yv, gV, dM, axd, jh, eq, jE, aGW, wD, hX, dP, hd, F, afm, azq, akJ, hD, vB, wm, uD, vO, yF, uQ, ux, WZ, bHo, ff, wc, bps, cr, eZ, dN, aF, hp, iV, bz, es, Bv, yE, aED, Lb, qu, XO, oQ, y, kS, iu, rk, pC); #c1 (HLA-DQA2) c2(NP_064440) c3(HLA class II histocompatibility antigen, DQ alpha 2 chain precursor) c4(202429, 202430, 202431, 202432, 202433, 202434) c5(202435, 202436, 202437, 202438, 202439, 202440, 202441, 202442, 202443, 202444, 202445, 202446, 202447, 202448, 202449, 202450, 202451, 202452, 202453, 202454, 202455, 202456, 202457, 202458, 202459, 202460, 202461, 202462, 202463, 202464, 202465, 202466, 202467, 202468, 202469, 202470, 202471, 202472, 202473, 202474, 202475, 202476, 202477, 202478, 202479, 202480, 202481, 202482, 202483, 202484, 202485, 202486, 202487, 202488, 202489) c6( )c7(bL, bM, dk, if, al, jh, aA, GP, aeu, aj, qj, jE, qN, lR, V, pd, ai, o, J, iu, AW, cF, F, JD, qp, bJj, aT, hD, kU, lM, vO, yF, hM, blJ, mR, A, bn, Jl, aW, Y, lh, az, hl, bJi, lb, cr, kt, eZ, aF, hp, g, mo, pb, adz, ar, yl, Qu, lV, bm, awQ, bAD, eo, oQ, y, it, Wl, ao, pC); #c1 (HLA-DQB1) c2(NP_001230890) c3(HLA class II histocompatibility antigen, DQ beta 1 chain isoform 2 precursor) c4(86943, 86944) c5(86945, 86946, 86947, 86948, 86949, 86950, 86951, 86952, 86953, 86954, 86955, 86956, 86957, 86958, 86959, 86960, 86961, 86962, 86963, 86964, 86965, 86966, 86967, 86968, 86969, 86970, 86971) c6( )c7(bM, T, xS, fR, bJm, if, al, fd, aeu, cL, ur, ci, qj, bh, aGX, Jt, fw, aY, Tz, lR, aMB, ip, NF, lF, AW, xy, xE, aX, fS, bJj, dV, aU, ja, rB, DY, hH, LG, eL, bc, hm, aa, li, dH, Jl, aQ, blP, bn, mQ, hl, HA, ih, aaN, hN, kt, aB, g, aFU, ve, uC, eE, GR, fT, iT, Fh, dc, uy, ki, mx, vy, cj, dW, bAD, k, m, pw, lS, eg, qm, aZN, sr, dD, QO, wu, vE, Hm, aw, yG, bzb, aFc, fN, blG, n, blD, aj, ez, apt, uE, aYv, qN, fr, qW, ai, jw, tD, xC, ft, cc, vM, i, aMj, kH, h, dL, ey, ux, Cr, mJ, alY, nP, OV, mR, A, sp, aRL, lb, CM, bJl, Ni, fe, aq, yq, aMJ, CQ, mo, gG, dw, cM, vo, Zj, Wt, aBs, ar, blF, blH, W, dB, hk, mg, Be, JM, uz, bm, Xa, fL, aDE, aCD, bni, lB, it, B, abW, bJp, bL, blR, jv, eR, dk, fG, jx, Nt, GP, aA, cz, OY, j, ajM, wy, V, eA, J, o, eJ, DJ, ady, mj, bJr, cF, blC, aEE, fh, bJn, nX, jZ, sl, bza, yg, aT, vH, cy, qp, hK, lM, sX, aZQ, to, ak, hM, iU, xR, Y, ayU, Pg, bg, r, axu, afq, aHU, lo, aDm, en, bu, aaO, t, pb, dS, ck, ajl, cT, BU, bJk, sn, OW, Dy, hA, ig, iQ, mG, of, AH, lV, auT, mf, dy, Da, gT, bt, aDj, ao, yv, Kd, bJq, aFe, akd, qb, aD, iW, axd, jh, wV, ah, jE, eq, bCx, aGW, hX, dP, hd, F, afm, ds, qh, akJ, oj, oW, dm, hD, rN, JK, vB, wm, ji, uD, vO, yF, uQ, uX, bHo, lU, lR, ff, aGS, bps, cr, eZ, dN, hv, Mz, aF, hp, Pd, iV, es, gk, Bv, DT, eU, yE, blS, arE, Ny, dO, aED, bE, Lj, aCl, aDB, gy, Lb, bJo, qu, oQ, afT, y, XO, WX, ME, rk, pC); #c1 (HLA-DQB1) c2(NP_001230891) c3(HLA class II histocompatibility antigen, DQ beta 1 chain isoform 1 precursor) c4(86972, 86973, 86974, 86975, 86976, 86977, 86978, 86979, 86980, 86981, 86982, 86983, 86984, 86985, 86986, 86987, 86988, 86989, 86990, 86991, 86992, 86993, 86994, 86995) c5(86996, 86997, 86998, 86999, 87000, 87001, 87002, 87003, 87004, 87005, 87006, 87007, 87008, 87009, 87010, 87011, 87012, 87013, 87014, 87015, 87016, 87017, 87018, 87019, 87020, 87021, 87022, 87023, 87024, 87025, 87026, 87027, 87028, 87029, 87030, 87031, 87032, 87033, 87034, 87035, 87036, 87037, 87038, 87039, 87040, 87041, 87042, 87043, 87044, 87045, 87046, 87047, 87048, 87049, 87050, 87051, 87052, 87053, 87054, 87055, 87056, 87057, 87058, 87059, 87060, 87061, 87062, 87063, 87064, 87065, 87066, 87067, 87068, 87069, 87070, 87071, 87072, 87073, 87074, 87075, 87076, 87077, 87078, 87079, 87080, 87081, 87082, 87083, 87084, 87085, 87086, 87087, 87088, 87089, 87090, 87091, 87092, 87093, 87094, 87095, 87096, 87097, 87098, 87099, 87100, 87101, 87102, 87103, 87104, 87105, 87106, 87107, 87108, 87109, 87110, 87111, 87112, 87113, 87114, 87115, 87116, 87117, 87118, 87119) c6( )c7(bM, T, xS, fR, bJm, if, al, fd, aeu, cL, ur, ci, qj, bh, aGX, Jt, fw, aY, Tz, lR, aMB, ip, NF, lF, AW, xy, xE, ax, FS, bJj, dV, aU, ja, rB, DY, hH, LG, eL, bc, hm, aa, li, dH, Jl, aW, blP, bn, mQ, hl, HA, ih, aaN, hN, kt, aB, g, aFU, ve, uC, eE, GR, fT, iT, Fh, dc, uy, ki, mx, vy, cj, dW, bAD, k, m, pw, lS, eg, qm, aZN, sr, dD, QO, wu, vE, Hm, aw, yG, bzb, aFc, fN, blG, n, blD, aj, ez, apt, uE, aYv, qN, fr, qW, ai, jw, tD, xC, ft, cc, vM, i, aMj, kH, h, dL, ey, ux, Cr, mJ, alY, nP, OV, mR, A, sp, aRL, lb, CM, bJl, Ni, fe, aq, yq, aMJ, CQ, mo, gG, dw, cM, vo, Zj, Wt, aBs, ar, blF, blH, W, dB, hk, mg, Be, JM, uz, bm, Xa, fL, aDE, aCD, bni, lB, it, B, abW, bJp, bL, blR, jv, eR, dk, fG, jx, Nt, GP, aA, cz, OY, j, ajM, wy, V, eA, J, o, eJ, DJ, ady, mj, bJr, cF, blC, aEE, fh, bJn, nX, jZ, sl, bza, yg, aT, vH, cy, qp, hK, lM, sX, aZQ, to, ak, hM, iU, xR, Y, ayU, Pg, bg, r, axu, afq, aHU, lo, aDm, en, bu, aaO, t, pb, dS, ck, ajl, cT, BU, bJk, sn, OW, Dy, hA, ig, iQ, mG, of, AH, lV, auT, mf, dy, Da, gT, bt, aDj, ao, yv, Kd, bJq, aFe, akd, qb, aD, iW, axd, jh, wV, ah, jE, eq, bCx, aGW, hX, dP, hd, F, afm, ds, qh, akJ, oj, oW, dm, hD, rN, JK, vB, wm, ji, uD, vO, yF, uQ, uX, bHo, lU, lR, ff, aGS, bps, cr, eZ, dN, hv, Mz, aF, hp, Pd, iV, es, gk, Bv, DT, eU, yE, blS, arE, Ny, dO, aED, bE, Lj, aCl, aDB, gy, Lb, bJo, qu, oQ, afT, y, XO, WX, ME, rk, pC); #c1(HLA-DQB1) c2(NP_002114) c3(HLA class II histocompatibility antigen, DQ beta 1 chain isoform 1 precursor) c4(125365, 125366, 125367, 125368, 125369) c5(125370, 125371, 125372, 125373, 125374, 125375, 125376, 125377, 125378, 125379, 125380, 125381, 125382, 125383, 125384, 125385, 125386, 125387, 125388, 125389, 125390, 125391, 125392, 125393, 125394, 125395, 125396, 125397, 125398, 125399, 125400, 125401, 125402, 125403, 125404, 125405, 125406, 125407, 125408, 125409, 125410, 125411, 125412, 125413, 125414, 125415, 125416, 125417, 125418, 125419, 125420, 125421, 125422, 125423, 125424, 125425, 125426, 125427, 125428, 125429) c6( )c7(bM, T, xS, fR, bJm, if, al, fd, aeu, cL, ur, ci, qj, bh, aGX, Jt, fw, aY, Tz, lR, aMB, ip, NF, lF, AW, xy, xE, ax, fS, bJj, dV, aU, ja, rB, DY, hH, LG, eL, bc, hm, aa, li, dH, Jl, aW, blP, bn, mQ, hl, HA, ih, aaN, hN, kt, aB, g, aFU, ve, uC, eE, GR, fT, iT, Fh, dc, uy, ki, mx, vy, cj, dW, bAD, k, m, pw, lS, eg, qm, aZN, sr, dD, QO, wu, vE, Hm, aw, yG, bzb, aFc, fN, blG, n, blD, aj, ez, apt, uE, aYv, qN, fr, qW, ai, jw, tD, xC, ft, cc, vM, i, aMj, kH, h, dL, ey, ux, Cr, mJ, alY, nP, OV, mR, A, sp, aRL, lb, CM, bJl, Ni, fe, aq, yq, aMJ, CQ, mo, gG, dw, cM, vo, Zj, Wt, aBs, ar, blF, blH, W, dB, hk, mg, Be, JM, uz, bm, Xa, fL, aDE, aCD, bni, lB, it, B, abW, bJp, bL, blR, jv, eR, dk, fG, jx, Nt, GP, aA, cz, OY, j, ajM, wy, V, eA, J, o, eJ, DJ, ady, mj, bJr, cF, blC, aEE, fh, bJn, nX, jZ, sl, bza, yg, aT, vH, cy, qp, hK, lM, sX, aZQ, to, ak, hM, iU, xR, Y, ayU, Pg, bg, r, axu, afq, aHU, lo, aDm, en, bu, aaO, t, pb, dS, ck, ajl, cT, BU, bJk, sn, OW, Dy, hA, ig, iQ, mG, of, AH, lV, auT, mf, dy, Da, gT, bt, aDj, ao, yv, Kd, bJq, aFe, akd, qb, aD, iW, axd, jh, wV, ah, jE, eq, bCx, aGW, hX, dP, hd, F, afm, ds, qh, akJ, oj, oW, dm, hD, rN, JK, vB, wm, ji, uD, vO, yF, uQ, uX, bHo, lU, lR, ff, aGS, bps, cr, eZ, dN, hv, Mz, aF, hp, Pd, iV, es, gk, Bv, DT, eL, yE, blS, arE, Ny, do, aED, bE, Lj, aCl, aDB, gy, Lb, bJo, qu, oQ, afT, y, XO, WX, ME, rk, pC); #c1(HLA-DQB2) c2(NP_001185787) c3(HLA class II histocompatibility antigen, DQ beta 2 chain isoform 2 precursor) c4(76354, 76355, 76356, 76357) c5(76358, 76359, 76360, 76361, 76362, 76363, 76364, 76365, 76366, 76367, 76368, 76369, 76370, 76371, 76372, 76373, 76374, 76375, 76376, 76377, 76378, 76379, 76380, 76381, 76382, 76383, 76384, 76385, 76386, 76387, 76388, 76389, 76390, 76391, 76392, 76393, 76394, 76395, 76396, 76397, 76398, 76399, 76400, 76401, 76402, 76403, 76404, 76405, 76406, 76407, 76408, 76409, 76410) c6( )c7(bM, T, xS, bJm, if, aeu, lR, bh, aGX, aY, Tz, aMB, ip, NF, lF, xy, xE, bJj, aU, hH, bc, li, dH, Jl, aW, bn, kt, g, eE, y, dc, mx, cj, bAD, qm, aZN, QO, vE, yG, aFc, n, aj, qN, h, ey, alY, nP, A, aRL, bJl, fe, mo, gG, Wt, mg, uz, it, B, bJp, bL, dk, Nt, GP, aA, OY, j, V, eJ, o, J, mj, cF, aEE, bJn, bza, aT, lM, sX, hM, xR, Y, ayU, lo, aDm, en, pb, ajl, cT, OW, Dy, hA, ig, lV, of, AH, mf, ao, Kd, bJq, akd, wV, jE, hX, dP, F, od, dm, hD, rN, vB, yF, uQ, lR, ff, cr, eZ, aF, hp, DT, eU, Lj, Ny, aDB, gy, XO, bJo, oQ, lf, hv); #c1(HLA-DQB2) c2(NP_001287719) c3(HLA class II histocompatibility antigen, DQ beta 2 chain isoform 1 precursor) c4(112641) c5(112642, 112643, 112644, 112645, 112646, 112647, 112648, 112649, 112650, 112651, 112652, 112653, 112654, 112655) c6( )c7(bM, T, xS, bJm, if, aeu, lR, bh, aGX, aY, Tz, aMB, ip, NF, lF, xy, xE, bJj, aU, hH, bc, li, dH, Jl, aW, bn, kt, g, eE, y, dc, mx, cj, bAD, qm, aZN, QO, vE, yG, aFc, n, aj, qN, h, ey, alY, nP, A, aRL, bJl, fe, mo, gG, Wt, mg, uz, it, B, bJp, bL, dk, Nt, GP, aA, OY, j, V, eJ, o, J, mj, cF, aEE, bJn, bza, aT, lM, sX, hM, xR, Y, ayU, lo, aDm, en, pb, ajl, cT, OW, Dy, hA, ig, lV, of, AH, mf, ao, Kd, bJq, akd, wV, jE, hX, dP, F, od, dm, hD, rN, vB, yF, uQ, lR, ff, cr, eZ, aF, hp, DT, eU, Lj, Ny, aDB, gy, XO, bJo, oQ, lf, hv); #c1(HLA-DQB2) c2(XP_005249108) c3(HLA class II histocompatibility antigen, DQ beta 2 chain isoform X13) c4(269029) c5(269030, 269031, 269032, 269033, 269034, 269035, 269036, 269037, 269038, 269039, 269040, 269041, 269042, 269043) c6( )c7(bM, T, xS, bJm, if, aeu, lR, bh, aGX, aY, Tz, aMB, ip, NF, lF, xy, xE, bJj, aU, hH, bc, li, dH, Jl, aW, bn, kt, g, eE, y, dc, mx, cj, bAD, qm, aZN, QO, vE, yG, aFc, n, aj, qN, h, ey, alY, nP, A, aRL, bJl, fe, mo, gG, Wt, mg, uz, it, B, bJp, bL, dK, Nt, GP, aA, DY, j, V, eJ, o, J, mj, cF, aEE, bJn, bza, aT, lM, sX, hM, xR, Y, ayU, lo, aDm, en, pb, ajl, cT, OW, Dy, hA, ig, lV, of, AH, mf, ao, Kd, bJq, akd, wV, jE, hX, dP, F, od, dm, hD, rN, vB, yF, uQ, lR, ff, cr, eZ, aF, hp, DT, eU, Lj, Ny, aDB, gy, XO, bJo, oQ, lf, hv); #c1(HLA-DQB2) c2(XP_011512863) c3(HLA class II histocompatibility antigen, DQ beta 2 chain isoform X15) c4(304114) c5(304115, 304116, 304117, 304118, 304119, 304120, 304121, 304122, 304123, 304124, 304125, 304126, 304127, 304128, 304129, 304130) c6( )c7(bM, T, xS, bJm, if, aeu, lR, bh, aGX, aY, Tz, aMB, ip, NF, lF, xy, xE, bJj, aU, hH, bc, li, dH, Jl, aW, bn, kt, g, eE, y, dc, mx, cj, bAD, qm, aZN, QO, vE, yG, aFc, n, aj, qN, h, ey, alY, nP, A, aRL, bJl, fe, mo, gG, Wt, mg, uz, it, B, bJp, bL, dk, Nt, GP, aA, OY, j, V, eJ, o, J, mj, cF, aEE, bJn, bza, aT, lM, sX, hM, xR, Y, ayU, lo, aDm, en, pb, ajl, cT, OW, Dy, hA, ig, lV, of, AH, mf, ao, Kd, bJq, akd, wV, jE, hX, dP, F, od, dm, hD, rN, vB, yF, uQ, lR, ff, cr, eZ, af, hp, DT, eU, Lj, Ny, aDB, gy, XO, bJo, oQ, lf, hv); #c1(HLA-DQB2) c2(XP_011546535) c3(HLA class II histocompatibility antigen, DQ beta 2 chain isoform X10) c4( )c5(345263, 345264, 345265, 345266) c6( )c7(bM, T, xS, bJm, if, aeu, lR, bh, aGX, aY, Tz, aMB, ip, NF, lF, xy, xE, bJj, aU, hH, bc, li, dH, Jl, aW, bn, kt, g, eE, y, dc, mx, cj, bAD, qm, aZN, QO, vE, yG, aFc, n, aj, qN, h, ey, alY, nP, A, aRL, bJl, fe, mo, gG, Wt, mg, uz, it, B, bJp, bL, dK, Nt, GP, aA, OY, j, V, eJ, o, J, mj, cF, aEE, bJn, bza, aT, lM, sX, hM, xR, Y, ayU, lo, aDm, en, pb, ajl, cT, OW, Dy, hA, ig, lV, of, AH, mf, ao, Kd, bJq, akd, wV, jE, hX, dP, F, od, dm, hD, rN, vB, yF, uQ, lR, ff, cr, eZ, aF, hp, DT, eU, Lj, Ny, aDB, gy, XO, bJo, oQ, lf, hv); #c1(HLA-DQB2) c2(XP_011546536) c3(HLA class I histocompatibility antigen, DQ beta 2 chain isoform X11) c4(345267) c5(345268, 345269, 345270, 345271, 345272, 345273, 345274, 345275, 345276, 345277, 345278, 345279, 345280, 345281, 345282, 345283, 345284, 345285, 345286, 345287, 345288, 345289, 345290, 345291) c6( )c7(bM, T, xS, bJm, if, aeu, lR, bh, aGX, aY, Tz, aMB, ip, NF, lF, xy, xE, bJj, aU, hH, bc, li, dH, Jl, aW, bn, kt, g, eE, y, dc, mx, cj, bAD, qm, aZN, QO, vE, yG, aFc, n, aj, qN, h, ey, alY, nP, A, aRL, bJl, fe, mo, gG, Wt, mg, uz, it, B, bJp, bL, dK, Nt, GP, aA, OY, j, V, eJ, o, J, mj, cF, aEE, bJn, bza, aT, lM, sX, hM, xR, Y, ayU, lo, aDm, en, pb, ajl, cT, OW, Dy, hA, ig, lV, of, AH, mf, ao, Kd, bJq, akd, wV, jE, hX, dP, F, od, dm, hD, rN, vB, yF, uQ, lR, ff, cr, eZ, aF, hp, DT, eU, Lj, Ny, aDB, gy, XO, bJo, oQ, lf, hv); #c1(HLA-DQB2) c2(XP_011546537) c3(HLA class II histocompatibility antigen, DQ beta 2 chain isoform X12) c4(345292, 345293, 345294) c5(345295, 345296, 345297, 345298, 345299, 345300, 345301, 345302, 345303, 345304, 345305, 345306, 345307, 345308, 345309, 345310, 345311, 345312, 345313, 345314, 345315, 345316, 345317, 345318, 345319, 345320, 345321, 345322, 345323, 345324, 345325, 345326, 345327, 345328, 345329, 345330, 345331, 345332, 345333, 345334, 345335, 345336, 345337, 345338, 345339, 345340, 345341) c6( )c7(bM, T, xS, bJm, if, aeu, lR, bh, aGX, aY, Tz, aMB, ip, NF, lF, xy, xE, bJj, aU, hH, bc, li, dH, Jl, aW, bn, kt, g, eE, y, dc, mx, cj, bAD, qm, aZN, QO, vE, yG, aFc, n, aj, qN, h, ey, alY, nP, A, aRL, bJl, fe, mo, gG, Wt, mg, uz, it, B, bJp, bL, dK, Nt, GP, aA, OY, j, V, eJ, o, J, mj, cF, aEE, bJn, bza, aT, lM, sX, hM, xR, Y, ayU, lo, aDm, en, pb, ajl, cT, OW, Dy, hA, ig, lV, of, AH, mf, ao, Kd, b, Jq, akd, wV, jE, hX, dP, F, od, dm, hD, rN, vB, yF, uQ, lR, ff, cr, eZ, aF, hp, DT, eU, Lj, Ny, aDB, gy, XO, bJo, oQ, lf, hv); #c1 (HLA-DRA) c2(NP_061984) c3(HLA class II histocompatibility antigen, DR alpha chain precursor) c4(201618, 201619, 201620, 201621, 201622, 201623, 201624, 201625, 201626, 201627) c5(201628, 201629, 201630, 201631, 201632, 201633, 201634, 201635, 201636, 201637, 201638, 201639, 201640, 201641, 201642, 201643, 201644, 201645, 201646, 201647, 201648, 201649, 201650, 201651, 201652, 201653, 201654, 201655, 201656, 201657, 201658, 201659, 201660, 201661, 201662, 201663, 201664, 201665, 201666, 201667, 201668, 201669, 201670, 201671, 201672, 201673, 201674, 201675, 201676, 201677, 201678, 201679, 201680, 201681, 201682, 201683, 201684, 201685, 201686, 201687, 201688, 201689, 201690, 201691, 201692, 201693, 201694, 201695, 201696, 201697, 201698, 201699, 201700, 201701, 201702, 201703, 201704, 201705, 201706, 201707, 201708, 201709, 201710, 201711, 201712, 201713, 201714, 201715, 201716, 201717, 201718, 201719, 201720, 201721, 201722, 201723, 201724, 201725, 201726, 201727, 201728, 201729, 201730, 201731, 201732, 201733, 201734, 201735, 201736, 201737, 201738, 201739, 201740, 201741, 201742, 201743, 201744, 201745, 201746, 201747, 201748, 201749, 201750, 201751, 201752, 201753, 201754, 201755, 201756, 201757, 201758, 201759, 201760, 201761, 201762) c6( )c7(bL, bM, wL, aFc, V, AW, ld, vH, h, ey, an, af, uQ, hM, A, sp, HA, eZ, r, Mz, hp, iV, pt, cM, pb, eM, Xa, y, WX, B); #c1(HLA-DRB1) c2(NP_001230894) c3(major histocompatibility complex, class II, DR beta 1 precursor) c4(87120, 87121) c5(87122, 87123, 87124, 87125, 87126, 87127, 87128, 87129, 87130, 87131, 87132, 87133, 87134, 87135, 87136, 87137, 87138, 87139, 87140) c6( )c7(T, bJN, fd, aeu, aoj, ci, aY, xy, rw, dV, iv, aFk, hH, aiJ, azh, aa, ly, uY, GR, fT, Jr, nB, dc, lp, cj, bAD, k, aZo, lS, vF, eg, uL, qm, sr, blB, bJF, aYZ, vE, ht, blD, cQ, aj, qW, cd, br, tD, aJe, vM, KN, ey, OM, mJ, nP, mR, bJl, sp, ge, fe, CQ, adz, dB, bJD, aCD, Wl, abW, bJp, jv, aA, GP, cz, wy, zn, aWb, akU, bJE, cy, jj, DX, lM, sX, Cy, dl, aFS, bf, lC, aMA, sV, bJC, lo, pb, Cv, ans, cT, OW, tW, ac, ig, iM, iQ, mG, hr, bt, gV, oq, bJq, iW, axd, aZU, jh, sJ, dG, uu, aYs, aue, ad, wD, aoH, hX, vJ, aFh, Dq, akJ, oW, lQ, wm, WZ, atJ, ff, oQ, afi, brp, iV, bz, aYt, DF, DT, Om, aDJ, ME, bJo, oQ, afT, lC, lf, WX, ne, rk, ra, lD, bM, xS, adP, if, e, cL, ur, bh, Tz, ip, yM, x, vV, fS, bFU, ja, azm, bc, uM, dH, Jl, hl, kt, aVO, aFU, bly, eE, bw, lt, xD, Fh, ki, ii, blQ, mx, vy, abh, xN, dD, bJs, wu, Hm, zt, yG, blG, n, oY, uE, jw, aMp, bJf, Mo, aMj, eV, DU, A, lb, ax, yq, vo, awQ, eo, fL, B, lH, eX, v, V, MK, sl, ld, ae, to, tN, aAi, hM, zH, aOK, axW, afS, afq, acB, bu, aaO, t, ck, auT, oX, Tu, vi, ob, dM, eq, aGW, BY, hd, od, afm, Db, ds, qh, LA, hD, rN, JK, bsn, uD, d, je, hG, wc, NH, dN, kD, dr, gk, yE, Ny, dO, gy, rH, y, hv, sH, pC, fR, al, qj, aGX, lR, Jt, bJe, aMB, AW, xE, bJG, bW, bJK, rB, K, wt, DY, dF, mH, aEV, blP, axu, mQ, hN, aB, g, pl, ve, uC, xK, uy, dW, m, bJv, Ro, NK, QO, aik, aw, fN, Ch, apt, pM, apz, eh, ft, aaA, blO, alY, aXy, aLE, cM, Ld, Zj, ar, blF, acC, blH, W, bbx, aZL, mg, JM, bzm, Xa, lB, Pr, it, blR, fM, bcb, fG, jx, Nt, wo, bJw, j, Vh, pd, eA, J, o, bur, bJO, lg, nX, jZ, hK, kl, xR, Y, ayU, Pg, r, aHU, en, bN, sn, aQd, hA, hc, of, uS, Da, dy, fD, gT, blu, Gl, am, CS, aZ, awz, ah, hF, gb, ase, dP, acu, awd, oj, vB, ji, lU, lR, bps, cr, hp, Fq, Pd, Bu, qa, bJt, bJz, Bv, Lj, aCl, qu, ei, hE, DS, bJx, eb, aTy, bwS, aFV, Jx, mF, aR, NF, dY, il, aU, hm, UC, aW, lh, HA, aaN, aVc, iT, qr, R, qR, afc, ez, fr, KO, qN, aPb, ai, ke, aJf, cc, wG, ql, h, jy, ux, OV, iw, aq, bJu, mo, gG, dw, acD, lx, oU, Wt, lX, hk, uw, bm, uz, aDE, bni, cn, bL, eR, dk, cR, aBo, ajM, OY, DJ, cF, ga, bJJ, blC, mP, bJA, qp, aT, vH, bJH, bJB, kU, af, aZQ, ak, iU, yL, OS, aDm, OC, wf, lY, ahN, bJk, wg, nM, yW, Zg, AH, aYx, Kd, qb, aD, ha, jE, mC, Uy, aiN, XB, qC, dZ, kL, F, oz, azq, bJL, alg, dm, vO, yF, uQ, bHo, bJM, aFE, aGS, azP, eZ, yy, aF, LB, ay, es, axs, atM, z, blS, Rv, aED, Lb, aS, XO, Lz, fh); #c1(HLA-DRB1) c2(NP_002115) c3(major histocompatibility complex, class II, DR beta I precursor) c4(125430, 125431, 125432, 125433, 125434) c5(125435, 125436, 125437, 125438, 125439, 125440, 125441, 125442, 125443, 125444, 125445, 125446, 125447, 125448, 125449, 125450, 125451, 125452, 125453, 125454, 125455, 125456, 125457, 125458, 125459, 125460, 125461, 125462, 125463, 125464, 125465) c6( )c7(T, bJN, fd, aeu, aoj, ci, aY, xy, rw, dV, iv, aFk, hH, aiJ, azh, aa, ly, uY, GR, fT, Jr, nB, dc, lp, cj, bAD, k, aZo, lS, vF, eg, uL, qm, sr, blB, bJF, aYZ, vE, ht, blD, cQ, aj, qW, cd, br, tD, ale, vM, KN, ey, OM, mJ, nP, mR, bJl, sp, ge, fe, CQ, adz, dB, bJD, aCD, Wl, abW, bJp, jv, aA, GP, cz, wy, zn, aWb, akU, bJE, cy, jj, DX, lM, sX, Cy, dl, aFS, bf, lC, aMA, sV, bJC, lo, pb, Cv, ans, cT, OW, tW, ac, ig, iM, iQ, mG, hr, bt, gV, oq, bJq, iW, axd, aZU, jh, sJ, dG, uu, aYs, aue, ad, wD, aoH, hX, vJ, aFh, Dq, akJ, oW, lQ, wm, WZ, atJ, ff, oO, afi, brp, iV, bz, aYt, DF, DT, Om, aDJ, ME, bJo, oQ, afT, lC, lf, WX, ne, rk, ra, lD, bM, xS, adP, if, e, cL, ur, bh, Tz, ip, yM, x, vV, fS, bFU, ja, azm, bc, uM, dH, Jl, hl, kt, aVO, aFU, bly, eE, bw, lt, xD, Fh, ki, ii, blQ, mx, vy, abh, xN, dD, bJs, wu, Hm, zt, yG, blG, n, oY, uE, jw, aMp, bJf, Mo, aMj, eV, DU, A, lb, ax, yq, vo, awQ, eo, fL, B, lH, eX, v, V, MK, sl, ld, ae, to, tN, aAi, hM, zH, aOK, axW, afS, afq, acB, bu, aaO, t, ck, auT, oX, Tu, vi, ob, dM, eq, aGW, BY, hd, od, afm, Db, ds, qh, LA, hD, rN, JK, bsn, uD, d, je, hG, wc, NH, dN, kD, dr, gk, yE, Ny, dO, gy, rH, y, hv, sH, pC, fR, al, qj, aGX, lR, Jt, bJe, aMB, AW, xE, bJG, bW, bJK, rB, K, wt, DY, dF, mH, aEV, blP, axu, mQ, hN, aB, g, pL, ve, uC, xK, uy, dW, m, bJv, Ro, NK, QO, aik, aw, fN, Qh, apt, pM, apz, eh, ft, aaA, blO, alY, aXy, aLE, cM, Ld, Zj, ar, blF, acC, blH, W, bbx, aZL, mg, JM, bzm, Xa, lB, Pr, it, blR, fM, bcb, fG, jx, Nt, wo, bJw, j, Vh, pd, eA, J, o, bJr, bJO, lg, nX, jZ, hK, kl, xR, Y, ayU, Pg, r, aHU, en, bN, sn, aQd, hA, hc, of, uS, Da, dy, fD, gT, blu, Gl, am, CS, aZ, awz, ah, hF, gb, ase, dP, acu, awd, oj, vB, ji, lU, lR, bps, cr, hp, Fq, Pd, Bu, qa, bJt, bJz, Bv, Lj, aCl, qu, ei, hE, DS, bJx, eb, aTy, bwS, aFV, Jx, mF, aR, NF, dY, il, aU, hm, UC, aW, lh, HA, aaN, aVc, iT, qr, R, qR, aFc, ez, fr, kO, qN, aPb, ai, ke, aJf, cc, wG, ql, h, jy, ux, OV, iw, aq, bJu, mo, gG, dw, acD, lx, ou, Wt, lX, hk, uw, bm, uz, aDE, bni, cn, bl, eR, dk, cR, aBo, ajM, OY, DJ, cF, ga, bJJ, blC, mP, bJA, qp, aT, vH, bJH, bJB, kU, af, aZQ, ak, iU, yL, OS, aDm, OC, wf, lY, ahN, bJk, wg, nM, yW, Zg, AH, aYx, Kd, qb, aD, ha, jE, mC, Uy, aiN, XB, qC, dZ, kL, F, oz, azq, bJL, alg, dm, vO, yF, uQ, bHo, bJM, aFE, aGS, azP, eZ, yy, aF, LB, ay, es, axs, atM, z, blS, Rv, aED, Lb, aS, XO, Lz, fh); #c1 (HLA-DRB3) c2(NP_072049) c3(major histocompatibility complex, class II, DR beta 3 precursor) c4(212568, 212569) c5(212570, 212571, 212572, 212573, 212574, 212575, 212576, 212577, 212578, 212579, 212580, 212581, 212582, 212583, 212584, 212585, 212586) c6( )c7(blR, jv, al, jx, cL, v, aoj, bh, eA, ai, o, DJ, cF, Dq, hD, h, DU, DY, uQ, ak, A, iU, eZ, hp, iV, aaO, qa, DT, sn, dB, bm); #c1(HLA-DRB4) c2(NP_068818) c3(major histocompatibility complex, class II, DR beta 4 precursor) c4(210818, 210819) c5(210820, 210821, 210822, 210823, 210824, 210825, 210826, 210827, 210828, 210829, 210830, 210831, 210832, 210833, 210834, 210835) c6( ) c7(oO, bM, T, xS, ea, aKu, if, al, eb, fd, cL, aoj, qj, ci, bh, aGX, lR, Tz, aR, bbo, x, AW, gJ, xy, vV, il, bW, fS, dV, ja, K, hu, hH, bc, dH, bn, aW, axu, az, hl, mQ, HA, aGQ, hN, bdk, as, akb, bJy, eF, cm, iP, bJP, y, lt, iA, Jr, lp, iT, cj, Zn, dW, k, m, Ro, P, dD, Hm, lo, yG, ht, ez, n, blD, aj, oY, fr, bd, qN, ai, bbB, ft, mh, bbt, bJS, aZv, ju, adX, h, UD, dL, ey, OM, mJ, alY, nP, OV, mR, CN, A, oo, aJ, sp, bJi, fe, yq, CQ, mo, zZ, aRJ, aBs, blF, adZ, bbx, w, aZL, mg, JM, bm, uz, Xa, fL, B, abW, bL, blR, bJT, fM, aYB, dK, jx, aA, cz, j, C, lu, V, pd, q, J, o, zn, DJ, er, eA, cF, bJn, ld, aT, yu, sX, ak, hM, kl, iU, vT, oV, Y, Pg, r, lo, en, hf, t, f, ik, pb, bN, OC, ck, ajl, cT, sS, bJk, sn, OW, kB, hA, ig, nM, iQ, of, AH, lV, auT, mf, dy, bko, bt, blu, vi, bjX, aZ, iW, jh, iD, Fb, eq, jE, oL, blM, mC, wD, hX, dP, hd, F, bJQ, vJ, ds, alg, hD, vB, yF, uQ, d, ue, hG, KS, aFE, azP, eH, cr, yy, eZ, ff, aF, hp, iV, bz, dr, es, gk, DT, pc, bE, Lj, XO, ME, i, hE, bJR, WX, hv, pC); #c1(HLA-DRB5) c2(NP_002116) c3(major histocompatibility complex, class II, DR beta 5 precursor) c4(125466) c5(125467, 125468, 125469, 125470, 125471, 125472, 125473) c6( )c7(bM, T, blR, fM, xS, if, jx, n, cl, aj, wV, bh, qN, lR, fr, wy, V, ME, Tz, o, eA, hX, NF, wD, cF, F, ds, bW, dV, hD, h, JK, lQ, an, aFk, bc, yF, uQ, ak, nP, A, XO, eZ, iU, je, oV, yL, mQ, afS, hl, r, aaN, Pg, lo, aF, hp, yq, cr, mo, dw, iV, pb, cT, bJk, sn, iM, ig, lV, of, mg, JM, bm, uz, bAD, bJo, dy, WX, eo, Xa, bJR, ZA, abW, y); #c1(HLA-E) c2(NP_005507) c3(HLA class I histocompatibility antigen, alpha chain E precursor) c4(156927, 156928, 156929) c5(156930, 156931, 156932, 156933, 156934, 156935, 156936, 156937, 156938, 156939, 156940, 156941, 156942, 156943, 156944, 156945, 156946, 156947, 156948, 156949, 156950, 156951, 156952, 156953, 156954, 156955, 156956, 156957, 156958, 156959, 156960, 156961, 156962, 156963) c6( )c7(DS, oO, T, bM, bzx, aC, fR, xS, bJW, ea, e, bkF, if, fd, bB, cL, qj, ci, bh, lR, mF, Tz, aMB, x, Vp, AW, bv, xy, xE, rw, aX, dV, ja, azm, rB, K, zy, hH, LG, eL, bc, DY, hm, bJX, akX, eG, axz, aW, lh, az, axu, awv, uY, hN, dg, g, as, eE, buy, uC, eF, mZ, cm, iP, ba, y, bJU, yl, eM, Jr, xD, dc, ki, iA, iT, mx, aiM, cj, bAD, k, m, gn, eg, zv, LT, dD, QO, JC, zt, aw, yG, qy, n, aj, oY, fr, ct, jX, uE, bfQ, qN, Tl, qW, cd, ai, eW, bbB, ft, cc, WB, vC, i, adX, cU, aMj, kH, dL, jy, ey, bav, aUH, h, mV, mY, on, mu, OV, asp, mR, A, sp, CM, lb, bJi, fe, aq, aMJ, CQ, mo, xe, Wt, mX, asE, bP, adZ, Qu, NL, aZL, lf, mg, abt, aDx, oh, uz, eo, aAl, fL, at, Xa, Tr, Pr, B, mT, cn, it, bL, Dw, jv, fM, dA, eR, fG, Ju, jx, eX, aA, aBo, mS, hz, v, j, bcE, V, pd, q, J, o, aGT, eA, ady, cF, fh, ld, yg, aT, yu, bK, Tj, jj, hK, kU, lM, sX, ae, bgp, ak, azU, hM, iU, oV, Y, aCH, bg, lC, eV, r, lo, Ph, axw, en, bu, aaO, t, ik, pb, bzc, bN, ck, lY, cT, sS, cL, OW, Dy, ig, vZ, awN, hc, bJY, of, iM, gX, lV, uS, Da, np, oX, gT, bt, zA, ao, vi, Kd, sY, aaT, hl, id, Ke, qb, LX, dM, iW, jh, Hn, wV, bS, eq, jE, iJ, mC, bCx, Uy, ad, qC, hX, afb, wD, F, od, mW, ds, Dq, alg, qh, oj, hD, vc, lQ, nJ, vO, yF, buh, uQ, d, lx, je, bJV, ff, aFE, xs, eH, cr, CF, yy, eZ, gE, hp, aF, Pd, Z, iV, bz, dr, kO, LB, es, Bv, bE, Lj, arE, eB, aS, rH, be, oQ, XO, HE, WX, X, hv, pC); #c1(HLA-F) c2(NP_001091948) c3(HLA class I histocompatibility antigen, alpha chain F isoform 3 precursor) c4(38804, 38805, 38806) c5(38807, 38808, 38809, 38810, 38811, 38812, 38813, 38814, 38815, 38816, 38817, 38818, 38819, 38820, 38821, 38822, 38823, 38824, 38825, 38826, 38827, 38828, 38829, 38830, 38831, 38832, 38833, 38834, 38835, 38836, 38837, 38838, 38839, 38840, 38841, 38842, 38843, 38844, 38845, 38846, 38847, 38848, 38849, 38850, 38851, 38852, 38853, 38854, 38855, 38856, 38857, 38858, 38859, 38860, 38861) c6( )c7(nJ, T, aOu, Y, eZ, it, yy, yu, iD, dL, h, ci, qN, bu, hH, V, eL, vi); #c1(HLA-F) c2(NP_001091949) c3(HLA class I histocompatibility antigen, alpha chain F isoform 1 precursor) c4(38862, 38863, 38864, 38865, 38866, 38867, 38868, 38869, 38870, 38871, 38872, 38873, 38874, 38875, 38876) c5(38877, 38878, 38879, 38880, 38881, 38882, 38883, 38884, 38885, 38886, 38887, 38888, 38889, 38890, 38891, 38892, 38893, 38894, 38895, 38896, 38897, 38898, 38899, 38900, 38901, 38902, 38903, 38904, 38905, 38906, 38907, 38908, 38909, 38910, 38911, 38912, 38913, 38914, 38915, 38916, 38917, 38918, 38919, 38920, 38921, 38922, 38923, 38924, 38925, 38926, 38927, 38928, 38929, 38930, 38931, 38932, 38933, 38934, 38935, 38936, 38937, 38938, 38939, 38940, 38941, 38942, 38943, 38944, 38945, 38946, 38947, 38948, 38949, 38950, 38951, 38952, 38953, 38954, 38955, 38956, 38957, 38958, 38959, 38960, 38961, 38962, 38963, 38964, 38965, 38966, 38967, 38968, 38969, 38970, 38971, 38972, 38973, 38974, 38975, 38976, 38977, 38978, 38979, 38980, 38981, 38982, 38983, 38984, 38985, 38986, 38987, 38988, 38989, 38990, 38991, 38992, 38993, 38994, 38995) c6( )c7(nJ, T, aOu, Y, eZ, it, yy, yu, iD, dL, h, ci, qN, bu, hH, V, eL, vi); #c1(HLA-F) c2(NP_061823) c3(HLA class I histocompatibility antigen, alpha chain F isoform 2 precursor) c4(200526, 200527, 200528, 200529, 200530) c5(200531, 200532, 200533, 200534, 200535, 200536, 200537, 200538, 200539, 200540, 200541, 200542, 200543, 200544, 200545, 200546, 200547, 200548, 200549, 200550, 200551, 200552, 200553, 200554, 200555, 200556, 200557, 200558, 200559, 200560, 200561, 200562, 200563, 200564, 200565, 200566, 200567, 200568, 200569, 200570, 200571, 200572, 200573, 200574, 200575, 200576, 200577, 200578, 200579, 200580, 200581, 200582, 200583, 200584, 200585, 200586, 200587, 200588, 200589, 200590, 200591, 200592, 200593, 200594, 200595, 200596, 200597, 200598, 200599, 200600, 200601, 200602, 200603, 200604, 200605, 200606) c6( )c7(nJ, T, aOu, Y, eZ, it, yy, yu, iD, dL, h, ci, qN, bu, hH, V, eL, vi); #c1(HLA-F) c2(XP_005272866) c3 (HLA class I histocompatibility antigen, alpha chain F isoform X1) c4(284204) c5( )c6( )c7(nJ, T, alu, Y, eZ, it, yy, yu, iD, dL, h, ci, qN, bu, hH, V, eL, vi); #c1(HLA-F) c2(XP_011545547) c3(HLA class I histocompatibility antigen, alpha chain F isoform X2) c4(344253, 344254) c5(344255, 344256, 344257, 344258, 344259, 344260, 344261, 344262, 344263, 344264, 344265, 344266, 344267, 344268, 344269, 344270, 344271, 344272, 344273, 344274, 344275, 344276) c6( )c7(nJ, T, aOu, Y, eZ, it, yy, yu, iD, dL, h, ci, qN, bu, hH, V, eL, vi): #c1(HLA-G) c2(NP_002118) c3(HLA class I histocompatibility antigen, alpha chain G precursor) c4(125489, 125490) c5(125491, 125492, 125493, 125494, 125495, 125496, 125497, 125498, 125499, 125500, 125501, 125502, 125503, 125504, 125505, 125506, 125507, 125508, 125509, 125510, 125511, 125512, 125513, 125514, 125515, 125516, 125517, 125518, 125519, 125520, 125521, 125522, 125523, 125524, 125525, 125526, 125527, 125528, 125529, 125530, 125531, 125532, 125533, 125534) c6( )c7(bM, G, T, fR, if, hE, eb, bB, fd, cL, ci, dV, ja, K, hu, DY, hH, eL, aW, axu, g, as, cm, uC, iP, iA, dc, mx, cj, Zn, m, FW, ht, n, fr, qN, jw, Vs, kH, h, eV, dL, OV, A, sp, lb, fe, pm, Ca, Zj, By, dB, hk, mg, JM, eo, gg, B, cn, jv, fM, jx, eX, aA, AO, V, q, eA, aT, XZ, hM, Y, r, lo, en, bu, t, ck, BU, nM, sn, fD, bt, hS, vi, sY, id, qb, iW, Jy, jh, Hn, hF, hX, dP, av, py, od, dm, hD, lQ, bsn, nJ, yF, uQ, d, ff, cr, eZ, hp, iV, bz, bE, lf, X, y); #c1(HLA-G) c2(XP_011546539) c3(HLA class I histocompatibility antigen, alpha chain G isoform X5) c4(345342, 345343, 345344) c5(345345, 345346, 345347) c6( )c7(bM, G, T, fR, if, hE, eb, bB, fd, cL, ci, dV, ja, K, hu, DY, hH, eL, aW, axu, g, as, cm, uC, iP, iA, dc, mx, cj, Zn, m, FW, ht, n, fr, qN, jw, Vs, kH, h, eV, dL, OV, A, sp, lb, fe, pm, Ca, Zj, Qy, dB, hk, mg, JM, eo, gg, B, cn, jv, fM, jx, eX, aA, AO, V, q, eA, aT, XZ, hM, Y, r, lo, en, bu, t, ck, BU, nM, sn, fD, bt, hS, vi, sY, id, qb, iW, Jy, jh, Hn, hF, hX, dP, av, py, od, dm, HD, lQ, bsn, nJ, yF, uQ, d, ff, cr, eL, hp, iV, bz, bE, lf, X, y); #c1(HLA-G) c2(XP_011546732) c3(HLA class I histocompatibility antigen, alpha chain G isoform X6) c4(345496) c5(345497, 345498, 345499, 345500, 345501, 345502, 345503, 345504, 345505, 345506, 345507, 345508, 345509, 345510, 345511, 345512, 345513, 345514, 345515, 345516, 345517, 345518, 345519, 345520, 345521, 345522, 345523, 345524, 345525, 345526, 345527, 345528, 345529, 345530, 345531, 345532, 345533, 345534, 345535, 345536) c6( )c7(bM, G, T, fR, if, hE, eb, bB, fd, cL, ci, dV, ja, K, hu, DY, hH, eL, aW, axu, g, as, cm, uC, iP, iA, dc, mx, cj, Zn, m, FW, ht, n, fr, qN, jw, Vs, kH, h, eV, dL, OV, A, sp, lb, fe, pm, Ca, Zj, Qy, dB, hk, mg, JM, eo, gg, B, cn, jv, fM, jx, eX, aA, AO, V, q, eA, aT, XZ, hM, Y, r, lo, en, bu, t, ck, BU, nM, sn, fD, bt, hS, vi, sY, id, qb, iW, Jy, jh, Hn, hF, hX, dP, av, py, od, dm, hD, lQ, bsn, nJ, yF, uQ, d, ff, cr, eZ, hp, iV, bz, bE, lf, X, y); #c1(HLA-G) c2(XP_011546733) c3(HLA class I histocompatibility antigen, alpha chain G isoform X7) c4(345537) c5(345538, 345539, 345540, 345541, 345542, 345543, 345544, 345545, 345546, 345547, 345548, 345549, 345550, 345551, 345552, 345553, 345554, 345555, 345556, 345557, 345558, 345559, 345560, 345561, 345562, 345563, 345564, 345565, 345566, 345567, 345568, 345569, 345570, 345571, 345572, 345573, 345574, 345575, 345576, 345577, 345578, 345579, 345580, 345581, 345582, 345583, 345584, 345585, 345586, 345587, 345588, 345589, 345590, 345591, 345592, 345593, 345594, 345595, 345596, 345597, 345598, 345599, 345600, 345601, 345602, 345603, 345604, 345605, 345606, 345607, 345608, 345609, 345610, 345611, 345612, 345613, 345614, 345615, 345616, 345617, 345618, 345619, 345620, 345621, 345622, 345623, 345624, 345625, 345626, 345627, 345628, 345629, 345630, 345631, 345632, 345633, 345634, 345635, 345636, 345637, 345638, 345639, 345640, 345641, 345642, 345643, 345644, 345645, 345646, 345647, 345648, 345649, 345650, 345651, 345652, 345653, 345654, 345655, 345656, 345657, 345658, 345659, 345660, 345661) c6( )c7(bM, G, T, fR, if, hE, eb, bB, fd, cL, ci, dV, ja, K, hu, DY, hH, eL, aW, axu, g, as, cm, uC, iP, iA, dc, mx, cj, Zn, m, FW, ht, n, fr, qN, jw, Vs, kH, h, eV, dL, OV, A, sp, lb, fe, pm, Ca, Zj, Qy, dB, hk, mg, JM, eo, gg, B, cn, jv, fM, jx, eX, aA, AO, V, q, eA, aT, XZ, HM, Y, r, lo, en, bu, t, ck, BU, nM, sn, fD, bt, hS, vi, sY, id, qb, iW, Jy, jh, Hn, hF, hX, dP, av, py, od, dm, hD, lQ, bsn, nJ, yF, ul, d, ff, cr, eZ, hp, iV, bz, bE, lf, X, y); #c1(HLCS) c2(XP_005261010) c3(biotin-protein ligase isoform X2) c4( )c5(277099, 277100) c6( )c7(BU, kh, PK, aW, oW, aT, zN, f, nJ, awp); #c1(HLCS) c2(XP_005261011) c3(biotin-protein ligase isoform X3) c4(277101, 277102, 277103) c5( ) c6( )c7(BU, kh, PK, aW, oW, aT, zN, f, nJ, awp); #c1(HLCS) c2(XP_011527843) c3(biotin-protein ligase isoform X1) c4(322780) c5(322781, 322782, 322783, 322784, 322785, 322786, 322787, 322788) c6( )c7(BU, kh, PK, aW, oW, aT, zN, f, nJ, awp); #c1(HLCS) c2(XP_011527844) c3(biotin-protein ligase isoform X5) c4( )c5(322789, 322790, 322791, 322792, 322793, 322794, 322795, 322796, 322797, 322798, 322799, 322800, 322801, 322802, 322803, 322804, 322805, 322806) c6( )c7(BU, kh, PK, aW, oW, aT, zN, f, nJ, awp); #c1(HLF) c2(NP_002117) c3(hepatic leukemia factor) c4(125474, 125475) c5(125476, 125477, 125478, 125479, 125480, 125481, 125482, 125483, 125484, 125485, 125486, 125487, 125488) c6( )c7(ck, x, ou, T, iO, ac, aE, aq, k, iR, pk, vt, B, azh); #c1(HLF) c2(XP_011523007) c3(hepatic leukemia factor isoform X2) c4( )c5(317492) c6( )c7(ck, x, ou, T, iO, ac, aE, aq, k, iR, pk, vt, B, azh); #c1(HLTF) c2(NP_003062) c3(helicase-like transcription factor) c4(134850, 134851) c5(134852, 134853, 134854, 134855, 134856, 134857, 134858, 134859, 134860, 134861, 134862, 134863, 134864, 134865, 134866, 134867, 134868, 134869, 134870, 134871, 134872) c6( )c7(bF, es, T, s, bE, if, ff, r, aA, bB, aF, ey, q, ci, en, qN, KV, X); #c1(HLTF) c2(XP_005247781) c3(helicase-like transcription factor isoform X3) c4(267982) c5(267983, 267984, 267985, 267986, 267987, 267988) c6( )c7(bF, es, T, s, bE, if, ff, r, aA, bB, aF, ey, q, ci, en, qN, KV, X); #c1 (HLX) c2(NP_068777) c3(H2,0-like homeobox protein) c4(210459) c5(210460, 210461, 210462, 210463, 210464, 210465, 210466, 210467, 210468) c6( )c7(ck, bM, T, x, iT, il, oe, wk, eZ, hD, k, pM, hF, bt); #c1(HM13) c2(NP_110416) c3(minor histocompatibility antigen H13 isoform 1) c4(220156, 220157) c5(220158, 220159, 220160, 220161, 220162, 220163, 220164, 220165, 220166, 220167, 220168, 220169) c6( )c7(bL, bM, T, dA, xR, bE, yx, dV, bB, h, nJ, af); #c1(HM13) c2(NP_848695) c3(minor histocompatibility antigen H13 isoform 2) c4( )c5(254/77, 254178, 254179, 254180, 254181, 254182) c6( )c7(bL, bM, T, dA, xR, bE, yx, dV, bB, h, nJ, af); #c1(HM13) c2(NP_848696) c3(minor histocompatibility antigen H13 isoform 3) c4(254183, 254184) c5(254185, 254186, 254187, 254188, 254189, 254190) c6( ) c7(bL, bM, T, dA, xR, bE, yx, dV, bB, h, nJ, af); #c1(HMBOX1) c2(NP_001129198) c3(homeobox-containing protein 1) c4(51138) c5(51139, 51140, 51141, 51142, 51143, 51144, 51145, 51146, 51147, 51148) c6( )c7(kS); #c1(HMBOX1) c2(XP_005273692) c3(homeobox-containing protein 1 isoform X2) c4(284803, 284804) c5(284805, 284806, 284807, 284808, 284809, 284810, 284811, 284812, 284813, 284814, 284815, 284816, 284817, 284818, 284819, 284820) c6( )c7(kS); #c1(HMBOX1) c2(XP_006716459) c3(homeobox-containing protein I isoform X1) c4(290329) c5( )c6( )c7(kS); #c1(HMBOX1) c2(XP_011542943) c3(homeobox-containing protein I isoform X9) c4(341638) c5(341639, 341640, 341641, 341642, 341643, 341644) c6( )c7(kS); #c1(HMBOX1) c2(XP_011542944) c3(homeobox-containing protein I isoform X10) c4( )c5(341645, 341646, 341647, 341648, 341649, 341650) c6( )c7(kS); #c1(HMBS) c2(NP_000181) c3(porphobilinogen deaminase isoform 1) c4(3907, 3908) c5(3909, 3910, 3911, 3912, 3913, 3914, 3915, 3916, 3917, 3918, 3919, 3920, 3921, 3922, 3923, 3924, 3925, 3926, 3927, 3928, 3929, 3930, 3931, 3932, 3933, 3934, 3935, 3936, 3937, 3938, 3939, 3940, 3941, 3942, 3943, 3944, 3945, 3946, 3947, 3948, 3949, 3950, 3951, 3952, 3953, 3954, 3955, 3956, 3957, 3958, 3959, 3960, 3961, 3962, 3963, 3964, 3965, 3966, 3967, 3968, 3969, 3970, 3971, 3972, 3973, 3974, 3975, 3976, 3977, 3978, 3979, 3980, 3981, 3982, 3983, 3984, 3985, 3986, 3987, 3988, 3989, 3990, 3991, 3992, 3993, 3994, 3995, 3996, 3997, 3998, 3999, 4000, 4001, 4002, 4003) c6( )c7(wu, QW, al, jh, bJZ, ti, Bm, ah, fu, bX, cc, DK, WY, aT, vc, bl, K, wm, bq, bKa, bV, nJ, iz, s, iU, Ow, aW, yL, hC, hl, dq, aq, dr, OC, ck, gk, aBr, eM, U, bqa, QV, uL, B, AG); #c1(HMBS) c2(NP_001245137) c3(porphobilinogen deaminase isoform 3) c4(92567, 92568, 92569) c5(92570, 92571, 92572, 92573, 92574, 92575, 92576, 92577, 92578, 92579, 92580, 92581) c6( )c7(wu, QW, al, jh, bJZ, ti, Bm, ah, fu, bX, cc, DK, WY, aT, vc, bl, K, wm, bq, bKa, bV, nJ, iz, s, iU, Ow, aW, yL, hC, hl, dq, aq, dr, OC, ck, gk, aBr, eM, U, bqa, QV, uL, B, AG); #c1(HMBS) c2(NP_001245138) c3(porphobilinogen deaminase isoform 4) c4( )c5(92582, 92583, 92584, 92585, 92586, 92587, 92588, 92589, 92590, 92591, 92592, 92593, 92594, 92595, 92596, 92597) c6( )c7(wu, QW, al, jh, bJZ, ti, Bm, ah, fu, bX, cc, DK, WY, aT, vc, bl, K, wm, bq, bKa, bV, nJ, iz, s, iU, Ow, aW, yL, hC, hl, dq, aq, dr, OC, ck, gk, aBr, eM, U, bqa, QV, uL, B, AG); #c1(HMBS) c2(XP_005271589) c3 (porphobilinogen deaminase isoform X1) c4( )c5(283487, 283488, 283489, 283490, 283491, 283492, 283493, 283494, 283495, 283496, 283497, 283498, 283499, 283500) c6( )c7(wu, QW, al, jh, bJZ, ti, Bm, ah, fu, bX, cc, DK, WY, aT, vc, bl, K, wm, bq, bKa, bV, nJ, iz, s, iU, Ow, aW, yL, hC, hl, dq, aq, dr, OC, ck, gk, aBr, eM, U, bqa, QV, uL, B, AG); #c1(HMCN1) c2(NP_114141) c3(hemicentin-1 precursor) c4(222316) c5(222317, 222318, 222319, 222320, 222321, 222322) c6( )c7(dD, ac, W, wL, ZC, EQ, eq); #c1(HMCN1) c2(XP_011508340) c3(hemicentin-1 isoform X2) c4(299023, 299024) c5(299025, 299026, 299027, 299028, 299029, 299030, 299031, 299032, 299033, 299034, 299035, 299036) c6( )c7(dD, ac, W, wL, ZC, EQ, eq); #c1(HMCN1) c2(XP_011508341) c3 (hemicentin-1 isoform X3) c4( )c5(299037) c6( )c7(dD, ac, W, wL, ZC, EQ, eq); #c1(HMG20A) c2(NP_001291433) c3(high mobility group protein 20A isoform a) c4(116016, 116017, 116018, 116019) c5(116020, 116021, 116022, 116023, 116024, 116025, 116026, 116027, 116028, 116029, 116030, 116031, 116032, 116033, 116034, 116035, 116036, 116037, 116038, 116039, 116040, 116041, 116042, 116043, 116044, 116045, 116046, 116047, 116048) c6( )c7(bL, v); #c1(HMG20B) c2(NP_006330) c3(SWI/SNF-related matrix-associated actin-dependent regulator of chromatin subfamily E member 1-related) c4(164070, 164071, 164072) c5(164073, 164074, 164075, 164076, 164077, 164078, 164079, 164080, 164081, 164082, 164083, 164084, 164085, 164086, 164087, 164088, 164089, 164090, 164091, 164092, 164093) c6( )c7(eA); #c1(HMGA1) c2(NP_665912) c3(high mobility group protein HMG-I/HMG-Y isoform b) c4(241977) c5(241978, 241979, 241980, 241981, 241982, 241983, 241984, 241985, 241986, 241987, 241988, 241989, 241990) c6( )c7(bL, T, azf, auW, Dh, wu, if, jh, bB, n, aj, dG, fr, ci, qN, AO, iJ, q, J, ad, eA, dP, eJ, av, hd, hw, aE, hW, pX, aT, h, rO, bbu, dL, hP, K, wm, ae, eL, jf, qw, yo, d, je, s, aW, Yd, PX, ff, r, en, hf, g, t, ir, bz, cm, op, ck, Qy, iM, kP, eM, nB, bE, eD, vA, U, hj, hR, m, ei, B); #c1(HMGA1) c2(XP_011512867) c3(high mobility group protein HMG-I/HMG-Y isoform X1) c4(304131, 304132) c5(304133, 304134, 304135, 304136, 304137, 304138, 304139, 304140, 304141, 304142, 304143, 304144, 304145, 304/46, 304147, 304148, 304149, 304150, 304151, 304152) c6( )c7(bL, T, azf, auW, Dh, wu, if, jh, bB, n, aj, dG, fr, ci, qN, AO, iJ, q, J, ad, eA, dP, eJ, av, hd, hw, aE, hW, pX, aT, h, rO, bbu, dL, hP, K, wm, ae, eL, jf, qw, yo, d, je, s, aW, Yd, PX, ff, r, en, hf, g, t, ir, bz, cm, op, ck, Qy, iM, kP, eM, nB, bE, eD, vA, U, hj, hR, m, ei, B); #c1(HMGA2) c2(NP_001287847) c3(high mobility group protein HMGI-C isoform c) c4(113019, 113020, 113021) c5(113022, 113023, 113024, 113025, 113026, 113027) c6( )c7(T, aC, aiH, e, bB, ci, aon, x, bv, aE, pX, bbu, K, eL, yo, aW, Qm, g, cm, bw, QA, qJ, eM, nB, bwr, vA, cj, m, k, pw, lT, iB, Or, qy, akt, br, hw, aex, h, dL, Dz, hB, ia, agF, pm, kk, op, aKA, bP, dB, eD, hj, hR, pg, B, bL, AS, bKc, aA, v, q, eA, qH, Zp, aHu, amg, mP, hb, aT, aHs, dz, rO, ae, GY, s, iU, Y, asy, LO, r, en, bu, t, iK, ck, QG, bKf, kP, au, dy, bKe, bKd, jh, bs, iJ, bKb, ad, afb, dP, av, hd, Db, io, hP, bwV, d, et, ff, aeV, aF, bz, bE, Fk, QC, fh); #c1(HMGA2) c2(NP_001287848) c3(high mobility group protein HMG1-C isoform d) c4(113028, 113029) c5(113030, 113031, 113032, 113033, 113034, 113035, 113036, 113037, 113038, 113039, 113040, 113041, 113042, 113043, 113044, 113045, 113046, 113047) c6( )c7(T, aC, aiH, e, bB, ci, aon, x, bv, aE, pX, bbu, K, eL, yo, aW, Qm, g, cm, bw, QA, qJ, eM, nB, bwr, vA, cj, m, k, pw, lT, iB, Or, qy, akt, br, hw, aeX, h, dL, Dz, hB, ia, agF, pm, kk, op, aKA, bP, dB, eD, hj, hR, pg, B, bL, AS, bKc, aA, v, q, eA, qH, Zp, aHu, amg, mP, hb, aT, aHs, dz, rO, ae, GY, s, iU, Y, asy, LO, r, en, bu, t, ik, ck, QG, bKf, kP, au, dy, bKe, bKd, jh, bs, iJ, bKb, ad, afb, dP, av, hd, Db, io, hP, bwV, d, et, ff, aeV, aF, bz, bE, Fk, QC, fh); #c1(HMGA2) c2(NP_003474) c3(high mobility group protein HMGI-C isoform a) c4(138625) c5(138626, 138627, 138628, 138629, 138630, 138631, 138632, 138633, 138634, 138635, 138636, 138637) c6( )c7(T, aC, aiH, e, bB, ci, aon, x, bv, aE, pX, bbu, K, eL, yo, aW, Qm, g, cm, bw, QA, qJ, eM, nB, bwr, vA, cj, m, k, pw, lT, iB, Or, qy, akt, br, hw, aex, h, dl, Dz, hB, ia, agF, pm, kk, op, aKA, bP, dB, eD, hj, hR, pg, B, bL, AS, bKc, aA, v, q, eA, qH, Zp, aHu, amg, mP, hb, aT, aHs, dz, rO, ae, GY, s, iU, Y, asy, LO, r, en, bu, t, ik, ck, QG, bKf, kP, au, dy, bKe, bKd, jh, bs, iJ, bKb, ad, afb, dP, av, hd, Db, io, hP, bwV, d, et, ff, aeV, aF, bz, bE, Fk, QC, fh); #c1(HMGA2) c2(NP_003475) c3(high mobility group protein HMGI-C isoform b) c4(138638, 138639, 138640) c5(138641, 138642, 138643, 138644, 138645, 138646, 138647, 138648, 138649, 138650, 138651, 138652, 138653, 138654, 138655, 138656, 138657, 138658, 138659, 138660, 138661, 138662, 138663, 138664, 138665, 138666, 138667, 138668, 138669) c6( )c7(T, aC, aiH, e, bB, ci, aon, x, bv, aE, pX, bbu, K, eL, yo, aW, Qm, g, cm, bw, QA, qJ, eM, nB, bwr, vA, cj, m, k, pw, lT, iB, Or, qy, akt, br, hw, aex, h, dL, Dz, hB, ia, agF, pm, kk, op, aKA, bP, dB, eD, hj, hR, pg, B, bL, AS, bKc, aA, v, q, eA, qH, Zp, aHu, amg, mP, hb, aT, aHs, dz, rO, ae, GY, s, iU, Y, asy, LO, r, en, bu, t, ik, ck, QG, bKf, kP, au, dy, bKe, bKd, jh, bs, iJ, bKb, ad, afb, dP, av, hd, Db, io, hP, bwV, d, et, ff, aeV, aF, bz, bE, Fk, QC, fh); #c1(HMGB1) c2(XP_005266425) c3(high mobility group protein B1 isoform X2) c4(280001) c5(280002, 280003, 280004, 280005, 280006, 280007, 280008, 280009, 280010, 280011, 280012, 280013, 280014, 280015, 280016, 280017, 280018, 280019, 280020, 280021, 280022, 280023, 280024, 280025, 280026, 280027, 280028, 280029, 280030) c6(280031, 280032, 280033, 280034, 280035, 280036, 280037) c7(oO, bM, T, lD, aC, fR, ea, eb, bB, cL, ci, x, bv, gH, M, K, dF, hm, Jl, LC, aW, g, eF, iP, aAW, y, eM, tV, k, m, yA, aFf, de, dD, ajm, aik, aw, RC, fN, n, aj, awr, cd, ai, b, Bj, KH, FN, h, wb, lb, fe, kp, jT, lx, ar, kT, dB, lf, bm, hj, fL, brs, it, B, fG, DV, aA, cR, cz, fF, j, C, il, V, q, J, o, eA, hb, sl, aT, cy, ae, af, eu, bf, iZ, Y, r, t, vk, ck, ac, AH, dy, fD, aws, bt, LS, ao, hS, kh, id, iW, ha, jh, eq, jE, atK, Fc, mC, ad, BY, hX, av, ib, py, Ot, ds, fb, uX, d, eH, eZ, aF, iV, aGj, bz, ek, Bv, zr, bE, blS, wz, i, lC, X, hv); #c1(HMGB2) c2(NP_001124161) c3(high mobility group protein B2) c4(48325) c5(48326, 48327, 48328, 48329, 48330, 48331, 48332, 48333, 48334, 48335, 48336, 48337, 48338, 48339, 48340, 48341, 48342, 48343, 48344, 48345, 48346) c6( )c7(ck, iu, T, gU, d, ND, nM, au, jh, r, vH, v, K, q, qN, B, o, lx); #c1(HMGB3) c2(NP_001288157) c3(high mobility group protein B3 isoform a) c4(113630) c5(113631, 113632, 113633, 113634, 113635, 113636, 113637, 113638, 113639, 113640, 113641, 113642, 113643, 113644, 113645, 113646, 113647, 113648, 113649, 113650, 113651, 113652, 113653, 113654, 113655, 113656) c6( )c7(ck, aW, T, eM, aT); #c1(HMGB3) c2(NP_001288160) c3(high mobility group protein B3 isoform b) c4(113657, 113658, 113659) c5(113660, 113661, 113662, 113663, 113664, 113665) c6( )c7(ck, aW, T, eM, aT); #c1(HMGCR) c2(NP_000850) c3(3-hydroxy-3-methylglutaryl-Coenzyme A reductase isoform 1) c4( )c5(16565, 16566, 16567, 16568, 16569, 16570, 16571, 16572, 16573, 16574, 16575, 16576, 16577, 16578) c6( )c7(bL, T, dD, eR, aZ, ea, e, aw, Xi, cz, bB, aj, v, j, eq, q, J, tg, ec, oj, aT, dV, lG, Di, dL, K, bq, dQ, af, hm, bKg, kl, cH, PE, aW, eH, r, da, aq, eF, qa, eY, ac, bE, Fi, at, ei, lC, bFT, B, y); #c1(HMGCR) c2(NP_001124468) c3(3-hydroxy-3-methylglutaryl-Coenzyme A reductase isoform 2) c4(49090, 49091) c5(49092, 49093, 49094, 49095, 49096, 49097, 49098, 49099, 49100, 49101, 49102, 49103, 49104, 49105, 49106, 49107) c6( ) c7(bL, T, dD, eR, aZ, ea, e, aw, Xi, cz, bB, aj, v, j, eq, q, J, tg, ec, oj, aT, dV, lG, Di, dL, K, bq, dQ, af, hm, bKg, kl, cH, PE, aW, eH, r, da, aq, eF, qa, eY, ac, bE, Fi, at, ei, lC, bFT, B, y); #c1(HMGCR) c2(XP_011541659) c3(3-hydroxy-3-methylglutaryl-Coenzyme A reductase isoform X2) c4(340199, 340200) c5(340201, 340202, 340203, 340204, 340205, 340206, 340207) c6( )c7(bL, T, dD, eR, aZ, ea, e, aw, Xi, cz, bB, aj, v, j, eq, q, J, tg, ec, oj, aT, dV, lG, Oi, dL, K, bq, dQ, af, hm, bKg, kl, cH, PE, aW, eH, r, da, aq, eF, qa, eY, ac, bE, Fi, at, ei, lC, bFT, B, y); #c1(HMGCR) c2(XP_011541661) c3(3-hydroxy-3-methylglutaryl-Coenzyme A reductase isoform X3) c4(340208, 340209) c5(340210, 340211, 340212) c6( )c7(bL, T, dD, eR, aZ, ea, e, aw, Xi, cz, bB, aj, v, j, eq, q, J, tg, ec, oj, aT, dV, lG, Di, dL, K, bq, dQ, af, hm, bKg, kl, cH, PE, aW, eH, r, da, aq, eF, qa, eY, ac, bE, Fi, at, ei, lC, bFT, B, y); #c1 (HMGCS1) c2(NP_002121) c3(hydroxymethylglutaryl-CoA synthase, cytoplasmic) c4(125535, 125536) c5(125537, 125538, 125539, 125540, 125541, 125542, 125543, 125544, 125545, 125546, 125547, 125548, 125549, 125550, 125551, 125552) c6( )c7(af, iW, aq, v, bq, B); #c1(HMGCS1) c2(XP_011512337) c3(hydroxymethylglutaryl-CoA synthase, cytoplasmic isoform X2) c4(302990) c5(302991, 302992, 302993, 302994, 302995, 302996) c6( )c7(af, iW, aq, v, bq, B); #c1 (HMGN1) c2(NP_004956) c3(non-histone chromosomal protein HMG-14) c4(151445, 151446) c5(151447, 151448, 151449, 151450, 151451, 151452, 151453, 151454, 151455, 151456, 151457) c6( )c7(lD, bN, hq, aW, acl, eZ, aT, jj, f, kS, Gc, lx, BY); #c1(HMGN1) c2(XP_011527851) c3 (non-histone chromosomal protein HMG-14 isoform X1) c4(322807) c5(322808, 322809, 322810, 322811, 322812, 322813, 322814) c6( )c7(lD, bN, hq, aW, acl, eZ, aT, jj, f, kS, Gc, lx, BY); #c1(HMGN4) c2(NP_006344) c3(high mobility group nucleosome-binding domain-containing protein 4) c4(164183) c5(164184, 164185, 164186, 164187, 164188, 164189, 164190, 164191, 164192, 164193) c6( )c7(T, ey); #c1 (HMGN5) c2(NP_110390) c3(high mobility group nucleosome-binding domain-containing protein 5) c4(219960, 219961, 219962, 219963, 219964) c5(219965, 219966, 219967, 219968, 219969, 219970, 219971, 219972, 219973, 219974, 219975, 219976, 219977, 219978, 219979, 219980, 219981, 219982, 219983, 219984, 219985, 219986, 219987, 219988) c6( )c7(ae, T, py, AM, aW, eL, eH, aT, dL, eF, fs, eA, kG, ad); #c1(HMGN5) c2(XP_006724753) c3(high mobility group nucleosome-binding domain-containing protein 5 isoform X3) c4(296309, 296310) c5(296311, 296312, 296313, 296314, 296315, 296316, 296317, 296318, 296319, 296320, 296321, 296322) c6( )c7(ae, T, py, AM, aW, eL, eH, al, dL, eF, fs, eA, kG, ad); #c1(HMGXB3) c2(NP_055798) c3(HMG domain-containing protein 3) c4(183570) c5(183571, 183572, 183573, 183574, 183575, 183576) c6( )c7(bv, an); #c1(HMGXB4) c2(NP_001003681) c3(HMG domain-containing protein 4) c4(19878, 19879) c5(19880, 19881, 19882, 19883, 19884, 19885) c6( )c7(mU, mW, mZ, mS, mY, bK, mV, mT, mX); #c1(HMGXB4) c2(XP_006724163) c3(HMG domain-containing protein 4 isoform X2) c4( )c5(295824) c6( )c7(mU, mW, mZ, mS, mY, bK, mV, mT, mX); #c1(HMGXB4) c2(XP_006724165) c3(HMG domain-containing protein 4 isoform X4) c4( )c5(295825, 295826, 295827, 295828, 295829) c6( )c7(mU, mW, mZ, mS, mY, bK, mV, mT, mX); #c1(HMHA1) c2(NP_001245257) c3(minor histocompatibility protein HA-1 isoform 2 precursor) c4(92902) c5(92903, 92904, 92905, 92906, 92907, 92908) c6( )c7(y, T, fL, fN); #c1(HMHA1) c2(NP_001269264) c3(minor histocompatibility protein HA-l isoform 4) c4(100994) c5(100995, 100996, 100997, 100998, 100999, 101000) c6( )c7(y, T, fL, fN); #c1(HMHA1) c2(NP_036424) c3(minor histocompatibility protein HA-1 isoform 1 precursor) c4(173733, 173734) c5(173735, 173736, 173737, 173738, 173739, 173740, 173741, 173742, 173743, 173744, 173745, 173746, 173747, 173748, 173749, 173750, 173751, 173752) c6( ) c7(y, T, fL, fN); #c1(HMHA1) c2(XP_006722776) c3(minor histocompatibility protein HA-1 isoform X3) c4(294868) c5(294869, 294870, 294871, 294872, 294873, 294874) c6( )c7(y, T, fL, fN); #c1(HMHA1) c2(XP_011526161) c3(minor histocompatibility protein HA-1 isoform X4) c4(320444, 320445) c5(320446, 320447, 320448, 320449, 320450, 320451) c6( )c7(y, T, fL, fN); #c1(HMMR) c2(NP_001136028) c3(hyaluronan mediated motility receptor isoform a) c4(54069, 54070) c5(54071, 54072, 54073, 54074, 54075, 54076, 54077, 54078, 54079, 54080, 54081, 54082) c6( )c7(T, fM, Uc, Js, jh, aA, bB, fd, ci, bX, q, ad, eA, eJ, x, aE, aT, dL, hP, ey, ae, eL, qw, kl, d, iUl, aW, ff, r, aF, en, as, ck, bE, go, hl, cj, bt, Jo); #c1(HMMR) c2(NP_001136029) c3(hyaluronan mediated motility receptor isoform d) c4(54083) c5(54084, 54085, 54086, 54087, 54088, 54089) c6( )c7(T, fM, Uc, Js, jh, aA, bB, fd, ci, bX, q, ad, eA, eJ, x, aE, aT, dL, hP, ey, ae, eL, qw, kl, d, iU, aW, ff, r, aF, en, as, ck, bE, go, hL, cj, bt, Jo); #c1(HMMR) c2(NP_036616) c3(hyaluronan mediated motility receptor isoform b) c4(174943) c5(174944, 174945, 174946, 174947, 174948, 174949) c6( )c7(T, fM, Uc, Js, jh, aA, bB, fd, ci, bX, q, ad, eA, eJ, x, aE, aT, dL, hP, ey, ae, eL, qw, kl, d, iU, aW, ff, r, aF, en, as, ck, bE, go, hL, cj, bt, Jo); #c1(HMMR) c2(NP_036617) c3(hyaluronan mediated motility receptor isoform c) c4(174950, 174951) c5(174952, 174953, 174954, 174955, 174956, 174957, 174958, 174959, 174960, 174961, 174962, 174963) c6( )c7(T, fM, Uc, Js, jh, aA, bB, fd, ci, bX, q, ad, eA, eJ, x, aE, aT, dL, hP, ey, ae, eL, qw, kl, d, iU, aW, ff, r, aF, en, as, ck, bE, go, hL, cj, bt, Jo); #c1(HMOX1) c2(NP_002124) c3(heme oxygenase 1) c4(125553, 125554) c5(125555, 125556, 125557, 125558, 125559, 125560, 125561, 125562, 125563, 125564, 125565, 125566, 125567) c6( )c7(oO, bM, T, lD, aC, fR, fV, ea, e, al, fd, bB, cL, qj, ci, aY, sD, GK, x, dY, aE, Bc, fm, rw, Yy, M, dV, K, hu, dQ, hH, dF, eL, hm, aa, ly, aaC, LC, aW, qS, az, hl, Jl, hC, fc, VE, aB, g, eE, cm, uC, xK, eM, iA, dc, wB, U, tV, bjL, mk, k, m, pw, eg, fy, iu, dD, aw, ht, n, aj, fr, ct, zP, aHj, eh, cd, eW, ke, Hx, jz, bKi, cc, bKk, h, lG, jy, dl, wb, ey, wo, eV, ZN, wC, A, ge, fQ, sm, aq, yq, fe, aMJ, jT, adz, lx, yR, ar, kT, JM, bm, at, E, uz, ER, gg, cB, B, it, L, bL, rf, DM, dA, eR, mr, DD, fG, uv, Lx, hx, jx, aA, cR, tc, cz, fF, j, uZ, C, AE, v, ex, V, q, J, o, eA, acj, MP, aT, cy, bq, an, ae, af, eu, jf, bf, s, iU, Y, r, lo, en, hf, t, iK, pb, ck, rR, iM, ac, bKh, c, wO, aUW, dh, AH, cS, dy, fD, bt, xb, bFR, LS, ao, bKj, nZ, qb, aD, uG, jh, lD, vr, KO, auq, bS, dG, eq, pz, hF, xt, ad, hX, fU, av, akN, F, l, ib, sO, ds, qh, Aa, lQ, ji, fb, vP, vl, mu, ux, d, je, cH, cW, ff, cr, eZ, dN, brp, aF, S, iV, Z, bz, qa, ek, LB, es, D, gk, qe, bE, X, sG, dX, aS, cP, ei, lC, gq, y, pC); #c1(HMOX2) c2(NP_001120676) c3(heme oxygenase 2 isoform b) c4( )c5(44497, 44498, 44499, 44500, 44501, 44502, 44503, 44504, 44505, 44506, 44507, 44508, 44509, 44510, 44511, 44512, 44513, 44514, 44515, 44516) c6( )c7(D, cZ, d, W, c, dD, sO, cz, fF, vs, eq, dd, af); #c1(HMOX2) c2(XP_011520775) c3(heme oxygenase 2 isoform X1) c4(315785) c5(315786, 315787, 315788, 315789, 315790, 315791) c6( )c7(D, cZ, d, W, c, dD, sO, cz, fF, vs, eq, dd, af); #c1(HMP19) c2(NP_057064) c3(neuron-specific protein family member 2) c4(188710, 188711, 188712) c5(188713, 188714, 188715, 188716, 188717, 188718, 188719, 188720, 188721, 188722, 188723, 188724, 188725, 188726, 188727, 188728, 188729, 188730, 188731, 188732, 188733, 188734) c6( )c7(aaw, j, anT); #c1(HMSD) c2(NP_001116838) c3(serpin-like protein HMSD) c4(43967) c5(43968, 43969, 43970, 43971, 43972, 43973, 43974, 43975, 43976, 43977, 43978, 43979) c6( )c7(iB, bS, afb); #c1(HMX1) c2(NP_061815) c3(homeobox protein HMX1) c4(200461) c5(200462, 200463, 200464, 200465, 200466, 200467, 200468) c6( )c7(kj, Az, bKl); #c1(HN1) c2(NP_001002032) c3(hematological and neurological expressed 1 protein isoform 2) c4(19412) c5(19413, 19414, 19415, 19416, 19417, 19418, 19419, 19420, 19421) c6( )c7(F, e, h, g, ae, ad); #c1(HN1) c2(NP_001275538) c3(hematological and neurological expressed I protein isoform 3) c4(106815) c5(106816, 106817, 106818, 106819, 106820, 106821, 106822, 106823, 106824, 106825) c6( ) c7(F, e, h, g, ae, ad); #c1(HN1) c2(NP_001275540) c3(hematological and neurological expressed 1 protein isoform 4) c4( )c5(106826) c6( )c7(F, e, h, g, ae, ad); #c1(HN1) c2(NP_057269) c3(hematological and neurological expressed I protein isoform 1) c4(189776, 189777) c5(189778, 189779, 189780, 189781, 189782) c6( )c7(F, e, h, g, ae, ad); #c1(HN1L) c2(NP_653171) c3(hematological and neurological expressed 1-like protein) c4(238907, 238908, 238909, 238910) c5(238911, 238912, 238913, 238914, 238915, 238916, 238917, 238918, 238919, 238920, 238921, 238922, 238923, 238924, 238925, 238926, 238927, 238928, 238929, 238930, 238931, 238932, 238933, 238934) c6( )c7(e); #c1(HNF1A) c2(NP_000536) c3(hepatocyte nuclear factor 1-alpha) c4(12178, 12179, 12180, 12181, 12182, 12183, 12184, 12185) c5(12186, 12187, 12188, 12189, 12190, 12191, 12192, 12193, 12194, 12195, 12196, 12197, 12198, 12199, 12200, 12201, 12202, 12203, 12204, 12205, 12206, 12207, 12208, 12209, 12210, 12211, 12212, 12213, 12214, 12215, 12216, 12217, 12218, 12219, 12220, 12221, 12222, 12223, 12224, 12225, 12226, 12227, 12228, 12229, 12230, 12231, 12232, 12233, 12234, 12235, 12236, 12237, 12238, 12239, 12240, 12241, 12242, 12243, 12244, 12245, 12246, 12247, 12248, 12249, 12250, 12251, 12252, 12253, 12254, 12255, 12256, 12257, 12258, 12259, 12260, 12261, 12262, 12263, 12264, 12265, 12266, 12267, 12268, 12269, 12270) c6( )c7(bL, gV, T, dD, bKq, bM, bqP, aC, eR, ea, Hm, e, bKo, yG, aw, aA, cz, bB, cQ, dG, j, ci, aj, v, eq, fr, GS, bKr, J, q, hX, mj, hd, mh, aE, Mm, py, lv, nX, bza, jy, wt, bKm, ex, on, rl, af, sj, A, d, bKp, s, cH, cW, fc, r, aq, aF, fd, iO, ac, nB, cO, bE, bKn, bo, X, KT, mg, hR, mf, aS, dy, ei, B, jr); #c1(HNF1A) c2(XP_005253988) c3(hepatocyte nuclear factor 1-alpha isoform X1) c4(272850) c5(272851, 272852, 272853, 272854, 272855, 272856) c6( )c7(bL, gV, T, dD, bKq, bM, bqP, aC, eR, ea, Hm, e, bKo, yG, aw, aA, cz, bB, cQ, dG, j, ci, aj, v, eq, fr, GS, bKr, J, q, hX, mj, hd, mh, aE, Mm, py, lv, nX, bza, jy, wt, bKm, ex, on, rL, af, sj, A, d, bKp, s, cH, cW, fc, r, aq, aF, fd, iO, ac, nB, cO, bE, bKn, bo, X, KT, mg, hR, mf, aS, dy, ei, B, jr); #c1(HNF1B) c2(NP_000449) c3(hepatocyte nuclear factor 1-beta isoform 1) c4(9118, 9119, 9120) c5(9121, 9122, 9123, 9124, 9125, 9126, 9127, 9128, 9129, 9130, 9131, 9132) c6( )c7(bL, nD, T, eR, bKu, byf, rQ, aMq, bKw, aj, akt, v, aoM, ci, vm, fr, aY, lu, J, ad, fU, nH, mh, yQ, uT, wG, tL, alb, il, arV, rx, bKs, h, Nj, uD, rz, ae, A, s, aBq, bKt, aeq, alB, bKv, cl, t, cM, dr, afj, bN, LB, bfP, amK, es, aKA, iA, nB, X, wB, hL, mf, dy, m, azg, B, VB); #c1(HNF1B) c2(NP_001159395) c3(hepatocyte nuclear factor-beta isoform 2) c4(63941) c5(63942, 63943, 63944, 63945, 63946, 63947) c6( )c7(bL, nD, T, eR, bKu, byf, rQ, aMq, bKw, aj, akt, v, aoM, ci, vm, fr, aY, lu, J, ad, fU, nH, mh, yQ, uT, wG, tl, alb, il, arV, rx, bKs, h, Nj, uD, rz, ae, A, s, aBq, bKt, aeq, alB, bKv, cl, t, cM, dr, aFj, bN, LB, bfP, amK, es, aKA, iA, nB, X, wB, hL, mf, dy, m, azg, B, VB); #c1(HNF1B) c2(NP_001291215) c3(hepatocyte nuclear factor 1-beta isoform 3) c4(115603) c5(115604, 115605, 115606, 115607, 115608, 115609) c6( )c7(bL, nD, T, eR, bKu, byf, rQ, aMq, bKw, aj, akt, v, aoM, ci, vm, fr, aY, lu, J, ad, fU, nH, mh, yQ, uT, wG, tl, alb, il, arV, rX, bKs, h, Nj, uD, rz, ae, A, s, aBq, bKt, aeq, alB, bKv, cl, t, cM, dr, afj, bN, LB, bfP, amK, es, aKA, iA, nB, X, wB, hL, mf, dy, m, azg, B, VB); #c1(HNF1B) c2(XP_011523462) c3(hepatocyte nuclear factor 1-beta isoform X1) c4( )c5(317998) c6( )c7(bL, nD, T, eR, bKu, byf, rQ, aMq, bKw, aj, akt, v, aoM, ci, vm, fr, aY, lu, J, ad, fU, nH, mh, yQ, uT, wG, tL, alb, il, arV, rx, bKs, h, Nj, uD, rz, ae, A, s, aBq, bKt, aeq, alB, bKv, cl, t, cM, dr, afj, bN, LB, bfP, amK, es, aKA, iA, nB, X, wB, hL, mf, dy, m, azg, B, VB); #c1(HNF1B) c2(XP_011523463) c3(hepatocyte nuclear factor 1-beta isoform X2) c4( )c5(317999, 318000, 318001, 318002, 318003) c6( )c7(bL, nD, T, eR, bKu, byf, rQ, aMq, bKw, aj, akt, v, aoM, ci, vm, fr, aY, lu, J, ad, fU, nH, mh, yQ, uT, wG, tL, alb, il, arV, rx, bKs, h, Nj, uD, rz, ae, A, s, aBq, bKt, aeq, alB, bKv, cl, t, cM, dr, aFj, bN, LB, bfP, amK, es, aKA, iA, nB, X, wB, hL, mf, dy, m, azg, B, VB); #c1 (HNF4A) c2(NP_000448) c3(hepatocyte nuclear factor 4-alpha isoform HNF4alpha2) c4(9109) c5(9110, 9111, 9112, 9113, 9114, 9115, 9116, 9117) c6( ) c7(T, ea, al, fd, bB, ci, u, K, eL, bc, aW, nY, as, g, hJ, eF, cm, eM, iA, iT, m, dD, zX, Hm, yG, aj, bKx, fr, mh, h, jy, dL, A, fe, aq, aKv, mb, bKy, JR, bbT, iO, Fi, bo, mg, hL, it, B, bL, eR, aA, cz, v, acn, il, q, J, eA, mj, Tl, nX, bza, aah, yg, aT, bq, ae, af, jf, s, vT, Y, bAv, r, hf, cl, t, mf, dy, bt, gV, ha, bAg, vm, dG, oZ, jE, fu, eq, bpT, ad, hX, av, l, QJ, qh, awd, aSf, ji, bKm, hG, eH, dq, aF, iV, iR, dr, bF, es, gk, mi, bE, gZ, dX, ei); #c1(HNF4A) c2(NP_001025174) c3(hepatocyte nuclear factor 4-alpha isoform HNF4alpha7) c4(29398) c5(29399, 29400, 29401, 29402, 29403, 29404) c6( )c7(T, ea, al, fd, bB, ci, u, K, eL, bc, aW, nY, as, g, hJ, eF, cm, eM, iA, iT, m, dD, zX, Hm, yG, aj, bKx, fr, mh, h, jy, dL, A, fe, aq, aKv, mb, bKy, JR, bbT, iO, Fi, bo, mg, hL, it, B, bL, eR, aA, cz, v, acn, il, q, J, eA, mj, Tl, nX, bza, aah, yg, aT, bq, ae, af, jf, s, vT, Y, bAv, r, hf, cl, t, mf, dy, bt, gV, ha, bAg, vm, dG, oZ, jE, fu, eq, bpT, ad, hX, av, l, QJ, qh, awd, aSf, ji, bKm, hG, eH, dq, aF, iV, iR, dr, bF, es, gk, mi, bE, gZ, dX, ei); #c1(HNF4A) c2(NP_001245284) c3(hepatocyte nuclear factor 4-alpha isoform HNF4alpha4) c4( )c5(92990) c6( )c7(T, ea, al, fd, bB, ci, u, K, eL, bc, aW, nY, as, g, hJ, eF, cm, eM, iA, iT, m, dD, zX, Hm, yG, aj, bKx, fr, mh, h, jy, dL, A, fe, aq, aKv, mb, bKy, JR, bbT, iO, Fi, bo, mg, hL, it, B, bL, eR, aA, cz, v, acn, il, q, J, eA, mj, Tl, nX, bza, aah, yg, aT, bq, ae, af, jf, s, vT, Y, bAv, r, hf, cl, t, mf, dy, bt, gV, ha, bAg, vm, dG, oZ, jE, fu, eq, bpT, ad, hX, av, l, QJ, qh, awd, aSf, ji, bKm, hG, eH, dq, aF, iV, iR, dr, bF, es, gk, mi, bE, gZ, dX, ei); #c1(HNF4A) c2(NP_787110) c3(hepatocyte nuclear factor 4-alpha isoform HNF4alpha8) c4(252103) c5(252104, 252105, 252106, 252107, 252108, 252109) c6( )c7(T, ea, al, fd, bB, ci, u, K, eL, bc, aW, nY, as, g, hJ, eF, cm, eM, iA, iT, m, dD, zX, Hm, yG, aj, bKx, fr, mh, h, jy, dL, A, fe, aq, aKv, mb, bKy, JR, bbT, iO, Fi, bo, mg, hL, it, B, bL, eR, aA, cz, v, acn, il, q, J, eA, mj, Tl, nX, bza, aah, yg, aT, bq, ae, af, jf, s, vT, Y, bAv, r, hf, cl, t, mf, dy, bt, gV, ha, bAg, vm, dG, oZ, jE, fu, eq, bpT, ad, hX, av, l, QJ, qh, awd, aSf, ji, bKm, hG, eH, dq, aF, iV, iR, dr, bF, es, gk, mi, bE, gZ, dX, ei); #c1(HNF4A) c2(NP_849180) c3(hepatocyte nuclear factor 4-alpha isoform HNF4alpha1) c4(254434, 254435) c5(254436, 254437, 254438, 254439, 254440, 254441) c6( )c7(T, ea, al, fd, bB, ci, u, K, eL, bc, aW, nY, as, g, hJ, eF, cm, eM, iA, iT, m, dD, zX, Hm, yG, aj, bKx, fr, mh, h, jy, dL, A, fe, aq, akv, mb, bKy, JR, bbT, iO, Fi, bo, mg, hL, it, B, bL, eR, aA, cz, v, acn, il, q, J, eA, mj, Tl, nX, bza, aah, yg, aT, bq, ae, af, jf, s, vT, Y, bAv, r, hf, cl, t, mf, dy, bt, gV, ha, bAg, vm, dG, oZ, jE, fu, eq, bpT, ad, hX, av, l, QJ, qh, awd, aSf, ji, bKm, hG, eH, dq, aF, iV, iR, dr, bF, es, gk, mi, bE, gZ, dX, ei); #c1(HNF4A) c2(NP_849181) c3(hepatocyte nuclear factor 4-alpha isoform HNF4alpha3) c4(254442) c5(254443, 254444, 254445, 254446, 254447, 254448, 254449, 254450, 254451, 254452) c6( )c7(T, ea, al, fd, bB, ci, u, K, eL, bc, aW, nY, as, g, hJ, ef, cm, eM, iA, iT, m, dD, zX, Hm, yG, aj, bKx, fr, mh, h, jy, dL, A, fe, aq, aKv, mb, bKy, JR, bbT, iO, Fi, bo, mg, hL, it, B, bL, eR, aA, cz, v, acn, il, q, J, eA, mj, Tl, nX, bza, aah, yg, aT, bq, ae, af, jf, s, vT, Y, bAv, r, hf, cl, t, mf, dy, bt, gV, ha, bAg, vm, dG, oZ, jE, fu, eq, bpT, ad, hX, av, l, QJ, qh, awd, aSf, ji, bKm, hG, eH, dq, aF, iV, iR, dr, bF, es, gk, mi, bE, gZ, dX, ei); #c1 (HNF4A) c2(XP_005260464) c3(hepatocyte nuclear factor 4-alpha isoform X3) c4(276547) c5(276548, 276549, 276550, 276551, 276552, 276553, 276554) c6( )c7(T, ea, al, fd, bB, ci, u, K, eL, bc, aW, nY, as, g, hJ, eF, cm, eM, iA, iT, m, dD, zX, Hm, yG, aj, bKx, fr, mh, h, jy, dL, A, fe, aq, aKv, mb, bKy, JR, bbT, iO, Fi, bo, mg, hL, it, B, bL, eR, aA, cz, v, acn, il, q, J, eA, mj, Tl, nX, bza, aah, yg, aT, bq, ae, af, jf, s, vT, Y, bAv, r, hf, cl, t, mf, dy, bt, gV, ha, bAg, vm, dG, oZ, jE, fu, eq, bpT, ad, hX, av, l, QJ, qh, awd, aSf, ji, bKm, hG, eH, dq, aF, iV, iR, dr, bF, es, gk, mi, bE, gZ, dX, ei); #c1(HNF4G) c2(NP_004124) c3(hepatocyte nuclear factor 4-gamma) c4(143766, 143767) c5(143768, 143769, 143770, 143771, 143772, 143773, 143774, 143775) c6( ) c7(aC, ac, EQ, aj, iV, fq, J); #c1(HNF4G) c2(XP_011515822) c3(hepatocyte nuclear factor 4-gamma isoform X1) c4( )c5(308626, 308627, 308628, 308629, 308630) c6( )c7(aC, ac, EQ, aj, iV, fq, J); #c1(HNMT) c2(NP_001019245) c3(histamine N-methyltransferase isoform 2) c4(27719) c5(27720, 27721, 27722, 27723, 27724, 27725) c6( )c7(bL, bM, T, Dw, uu, V, Gt, aE, fS, qp, aT, Ds, an, tN, aW, sp, aCx, r, iV, eE, xw, wg, JM, Dv); #c1(HNMT) c2(NP_001019246) c3(histamine N-methyltransferase isoform 3) c4(27726) c5(27727, 27728, 27729, 27730, 27731, 27732) c6( )c7(bL, bM, T, Dw, uu, V, Gt, aE, fS, qp, aT, Ds, an, tN, aW, sp, aCx, r, iV, eE, xw, wg, JM, Dv); #c1(HNMT) c2(NP_008826) c3(histamine N-methyltransferase isoform 1) c4(168851) c5(168852, 168853, 168854, 168855, 168856, 168857, 168858, 168859, 168860, 168861) c6( )c7(bL, bM, T, Dw, uu, V, Gt, aE, fS, qp, aT, Ds, an, tN, aW, sp, aCx, r, iV, eE, xw, wg, JM, Dv); #c1(HNRNPA0) c2(NP_006796) c3(heterogeneous nuclear ribonucleoprotein A0) c4(168154) c5(168155, 168156, 168157, 168158, 168159, 168160) c6( )c7(n, aF, aA); #c1(HNRNPA1) c2(NP_002127) c3(heterogeneous nuclear ribonucleoprotein A1 isoform a) c4(125568, 125569, 125570) c5(125571, 125572, 125573, 125574, 125575, 125576, 125577, 125578, 125579, 125580, 125581, 125582, 125583, 125584, 125585, 125586, 125587, 125588, 125589, 125590, 125591, 125592, 125593, 125594, 125595, 125596, 125597, 125598, 125599, 125600) c6( )c7(T, fR, fd, bB, bkz, ci, zP, hF, Fc, q, ad, WY, bKA, KH, kf, oj, h, rB, K, af, zQ, d, iU, r, aq, aHU, aaE, app, iR, ck, es, bb, c, eM, bE, iA, U, Da, bi, B); #c1(HNRNPA1) c2(XP_005268883) c3(heterogeneous nuclear ribonucleoprotein A1 isoform X1) c4(282160, 282161) c5( )c6( )c7(T, fR, fd, bB, bkz, ci, zP, hF, Fc, q, ad, WY, bKA, kH, kf, oj, h, rB, K, af, zQ, d, iU, r, aq, aHU, aaE, app, iR, ck, es, bb, c, eM, bE, iA, U, Da, bi, B); #c1(HNRNPA2B1) c2(NP_002128) c3(heterogeneous nuclear ribonucleoproteins A2/B1 isoform A2) c4(125601, 125602) c5(125603, 125604, 125605, 125606, 125607, 125608, 125609, 125610, 125611, 125612, 125613, 125614) c6( )c7(T, aA, pd, ad, av, aE, oj, aT, K, bq, af, d, bKB, Jl, aW, aq, kp, app, aF, iR, f, bz, c, eM, dB, hE, B, y); #c1(HNRNPAZB1) c2(XP_005249786) c3(heterogeneous nuclear ribonucleoproteins A2/B1 isoform X1) c4(269817) c5( )c6( )c7(T, aA, pd, ad, av, aE, oj, aT, K, bq, af, d, bKB, Jl, aW, aq, kp, app, aF, iR, f, bz, c, eM, dB, hE, B, y); #c1(HNRNPAZB1) c2(XP_006715777) c3(heterogeneous nuclear ribonucleoproteins A2/B1 isoform X2) c4( ) c5(289779, 289780, 289781, 289782, 289783, 289784, 289785, 289786, 289787, 289788) c6( )c7(T, aA, pd, ad, av, aE, oj, aT, K, bq, af, d, bKB, Jl, aW, aq, kp, app, aF, iR, f, bz, c, eM, dB, hE, B, y); #c1(HNRNPA3) c2(XP_005246437) c3(heterogeneous nuclear ribonucleoprotein A3 isoform X1) c4(267132) c5( )c6( )c7(nv, bxy); #c1(HNRNPA3) c2(XP_005246438) c3(heterogeneous nuclear ribonucleoprotein A3 isoform X2) c4(267133, 267134) c5(267135, 267136, 267137, 267138, 267139, 267140, 267141, 267142, 267143, 267144, 267145, 267146) c6( )c7(nv, bxy); #c1(HNRNPA3) c2(XP_006712428) c3(heterogeneous nuclear ribonucleoprotein A3 isoform X3) c4( )c5(286649, 286650, 286651, 286652, 286653, 286654, 286655, 286656, 286657, 286658) c6( )c7(nv, bxy); #c1(HNRNPAB) c2(NP_004490) c3(heterogeneous nuclear ribonucleoprotein A/B isoform b) c4(146869) c5(146870, 146871, 146872, 146873, 146874, 146875, 146876, 146877, 146878, 146879, 146880, 146881, 146882, 146883, 146884, 146885, 146886, 146887, 146888, 146889) c6( ) c7(zP, aGX, eM); #c1(HNRNPAB) c2(NP_112556) c3(heterogeneous nuclear ribonucleoprotein A/B isoform a) c4(221381) c5( )c6( )c7(zP, aGX, eM); #c1(HNRNPC) c2(XP_011535013) c3(heterogeneous nuclear ribonucleoproteins C1/C2 isoform X1) c4(331556, 331557, 331558, 331559, 331560, 331561, 331562) c5(331563, 331564, 331565, 331566, 331567, 331568, 331569, 331570, 331571, 331572, 331573, 331574, 331575, 331576, 331577, 331578, 331579, 331580, 331581, 331582, 331583, 331584, 331585, 331586, 331587, 331588, 331589, 331590, 331591, 331592, 331593, 331594, 331595, 331596, 331597, 331598, 331599, 331600, 331601, 331602, 331603, 331604, 331605, 331606, 331607, 331608, 331609, 331610, 331611, 331612, 331613, 331614) c6( )c7(ae, T, y, iM, eW, eM, je, dR, eZ, h, aq, ci, K, aGX, iR, cm, bz, B, ad, Qe); #c1(HNRNPC) c2(XP_011535014) c3(heterogeneous nuclear ribonucleoproteins C1/C2 isoform X2) c4(331615, 331616, 331617, 331618, 331619, 331620) c5(331621, 331622, 331623, 331624, 331625, 331626, 331627, 331628, 331629, 331630, 331631, 331632, 331633, 331634, 331635, 331636, 331637, 331638, 331639, 331640, 331641, 331642, 331643, 331644, 331645, 331646, 331647, 331648, 331649, 331650, 331651, 331652, 331653, 331654, 331655, 331656, 331657, 331658, 331659, 331660, 331661, 331662, 331663, 331664, 331665, 331666, 331667, 331668, 331669, 331670, 331671, 331672, 331673, 331674, 331675, 331676, 331677, 331678, 331679, 331680, 331681, 331682, 331683, 331684, 331685, 331686, 331687, 331688, 331689, 331690, 331691, 331692, 331693, 331694, 331695, 331696) c6( )c7(ae, T, y, iM, eW, eM, je, dR, eZ, h, aq, ci, K, aGX, iR, cm, bz, B, ad, Qe); #c1(HNRNPO) c2(NP_001003810) c3(heterogeneous nuclear ribonucleoprotein DO isoform d) c4(20169) c5(20170, 20171, 20172, 20173, 20174, 20175, 20176, 20177, 20178, 20179, 20180, 20181, 20182, 20183, 20184, 20185, 20186, 20187) c6( )c7(iu, T, hd, eR, eM, Y, ff, jx, Pg, eZ, en, B); #c1(HNRNPD) c2(NP_002129) c3(heterogeneous nuclear ribonucleoprotein DO isoform c) c4(125615, 125616) c5(125617, 125618, 125619, 125620, 125621, 125622, 125623, 125624, 125625, 125626, 125627, 125628) c6( )c7(iu, T, hd, eR, eM, Y, ff, jx, Pg, eZ, en, B); #c1(HNRNPD) c2(NP_112737) c3(heterogeneous nuclear ribonucleoprotein DO isoform b) c4(221569, 221570, 221571) c5(221572, 221573, 221574, 221575, 221576, 221577, 221578, 221579, 221580, 221581, 221582, 221583, 221584, 221585, 221586, 221587, 221588, 221589) c6( )c7(iu, T, hd, eR, eM, Y, ff, jx, Pg, eZ, en, B); #c1(HNRNPD) c2(NP_112738) c3(heterogeneous nuclear ribonucleoprotein DO isoform a) c4(221590, 221591, 221592) c5(221593, 221594, 221595, 221596, 221597, 221598) c6( )c7(iu, T, hd, eR, eM, Y, ff, jx, Pg, eZ, en, B); #c1(HNRNPDL) c2(NP_001193929) c3(heterogeneous nuclear ribonucleoprotein D-like isoform b) c4(83229) c5(83230, 83231, 83232, 83233, 83234, 83235) c6( )c7(ae, T, y, iM, eW, eM, je, dR, km, al, h, aq, eZ, ci, aGX, iR, cm, bz, B, ad, Qe); #c1(HNRNPDL) c2(NP_112740) c3(heterogeneous nuclear ribonucleoprotein D-like isoform a) c4(221607, 221608) c5(221609, 221610, 221611, 221612, 221613, 221614, 221615, 221616, 221617, 221618) c6( )c7(ae, T, y, iM, eW, eM, je, dR, km, aT, h, aq, eZ, ci, aGX, iR, cm, bz, B, ad, Qe); #c1(HNRNPF) c2(NP_001091676) c3 (heterogeneous nuclear ribonucleoprotein F) c4(38644, 38645) c5(38646, 38647, 38648, 38649, 38650, 38651, 38652, 38653, 38654, 38655, 38656, 38657, 38658, 38659, 38660, 38661, 38662, 38663, 38664, 38665, 38666, 38667, 38668, 38669, 38670, 38671, 38672, 38673) c6( )c7(bB, bE, B, bxy); #c1(HNRNPH1) c2(NP_005511) c3(heterogeneous nuclear ribonucleoprotein H) c4(156964, 156965) c5(156966, 156967, 156968, 156969, 156970, 156971, 156972, 156973, 156974, 156975, 156976, 156977, 156978, 156979) c6( )c7(xA, T, bb, aWm, c, ad, dR); #c1(HNRNPH1) c2(XP_006714926) c3(heterogeneous nuclear ribonucleoprotein H isoform X4) c4(288309) c5(288310, 288311, 288312, 288313, 288314, 288315, 288316, 288317, 288318, 288319, 288320, 288321, 288322, 288323, 288324, 288325, 288326, 288327, 288328, 288329, 288330, 288331, 288332, 288333, 288334, 288335, 288336, 288337, 288338, 288339, 288340, 288341, 288342, 288343, 288344, 288345, 288346, 288347, 288348, 288349, 288350, 288351, 288352, 288353, 288354, 288355, 288356, 288357, 288358, 288359, 288360, 288361, 288362, 288363) c6( ) c7(xA, T, bb, aWm, c, ad, dR); #c1 (HNRNPH1) c2(XP_011532848) c3(heterogeneous nuclear ribonucleoprotein H isoform X1) c4(328961, 328962, 328963, 328964, 328965) c5( ) c6( )c7(xA, T, bb, aWm, c, ad, dR); #c1(HNRNPH1) c2(XP_011532849) c3(heterogeneous nuclear ribonucleoprotein H isoform X2) c4(328966, 328967) c5( )c6( )c7(xA, T, bb, aWm, c, ad, dR); #c1(HNRNPH2) c2(NP_001027565) c3(heterogeneous nuclear ribonucleoprotein H2) c4(30277, 30278) c5(30279, 30280, 30281, 30282, 30283, 30284, 30285, 30286, 30287, 30288, 30289, 30290, 30291, 30292, 30293) c6( )c7(aWm, xA, T, bb, c, dR); #c1(HNRNPK) c2(NP_112553) c3(heterogeneous nuclear ribonucleoprotein K isoform a) c4(221352, 221353) c5(221354, 221355, 221356, 221357, 221358, 221359, 221360, 221361, 221362, 221363, 221364, 221365, 221366, 221367, 221368, 221369, 221370, 221371, 221372, 221373) c6( )c7(T, B, aE, aW, Y, ff, aT, fd, n, ci, en, bt, ae, q, ad); #c1(HNRNPL) c2(NP_001524) c3(heterogeneous nuclear ribonucleoprotein L isoform a) c4(118601, 118602) c5(118603, 118604, 118605, 118606, 118607, (18608) c6( )c7(aA, aT); #c1(HNRNPL) c2(XP_011525188) c3(heterogeneous nuclear ribonucleoprotein L isoform X2) c4( )c5(319643, 319644) c6( )c7(aA, aT); #c1(HNRNPL) c2(XP_011525191) c3(heterogeneous nuclear ribonucleoprotein L isoform X1) c4( )c5(319645, 319646, 319647, 319648, 319649, 319650, 319651, 319652, 319653) c6( )c7(aA, aT); #c1(HNRNPL) c2(XP_011525192) c3(heterogeneous nuclear ribonucleoprotein L isoform X5) c4(319654, 319655) c5(319656, 319657, 319658, 319659, 319660, 319661, 319662, 319663, 319664, 319665, 319666, 319667) c6( )c7(aA, aT); #c1(HNRNPM) c2(NP_005959) c3(heterogeneous nuclear ribonucleoprotein M isoform a) c4(160743, 160744) c5(160745, 160746, 160747, 160748, 160749, 160750, 160751, 160752, 160753, 160754, 160755) c6( )c7(T); #c1(HNRNPM) c2(NP_112480) c3(heterogeneous nuclear ribonucleoprotein M isoform b) c4(221128) c5(221129, 221130, 221131, 221132, 221133, 221134, 221135) c6( )c7(T); #c1(HNRNPM) c2(XP_005272535) c3(heterogeneous nuclear ribonucleoprotein M isoform X1) c4(284053) c5(284054, 284055, 284056, 284057, 284058, 284059) c6( )c7(T); #c1(HNRNPM) c2(XP_005272540) c3(heterogeneous nuclear ribonucleoprotein M isoform X5) c4( )c5(284060, 284061, 284062, 284063, 284064) c6( )c7(T); #c1(HNRNPR) c2(NP_001284549) c3(heterogeneous nuclear ribonucleoprotein R isoform 5) c4(112049) c5(112050, 112051, 112052, 112053, 112054, (12055) c6( )c7(kf, aT); #c1(HNRNPR) c2(NP_001284550) c3(heterogeneous nuclear ribonucleoprotein R isoform 7) c4( )c5(112056, 112057, 112058, 112059, 112060) c6( )c7(kf, aT); #c1(HNRNPR) c2(XP_011538773) c3(heterogeneous nuclear ribonucleoprotein R isoform X1) c4(336509) c5(336510, 336511, 336512, 336513, 336514, 336515) c6( )c7(kf, aT); #c1(HNRNPR) c2(XP_011538775) c3(heterogeneous nuclear ribonucleoprotein R isoform X4) c4(336516) c5(336517) c6( )c7(kf, aT); #c1(HNRNPU) c2(NP_004492) c3(heterogeneous nuclear ribonucleoprotein U isoform b) c4(146890) c5(146891, 146892, 146893, 146894, 146895, 146896, 146897, 146898, 146899, 146900, 146901, 146902) c6( )c7(T, aE, qt, aW, r, aT, iY, ci, eA, an, zy, AO, B); #c1(HNRNPU) c2(NP_114032) c3(heterogeneous nuclear ribonucleoprotein U isoform a) c4(222070, 222071, 222072) c5(222073, 222074, 222075, 222076, 222077, 222078, 222079, 222080, 222081, 222082, 222083, 222084) c6( )c7(T, aE, qt, aW, r, aT, iY, ci, eA, an, zy, AO, B); #c1(HNRNPUL1) c2(NP_008971) c3(heterogeneous nuclear ribonucleoprotein U-like protein I isoform a) c4(169978, 169979) c5( )c6( )c7(aw, ei, j, ec); #c1(HNRNPUL1) c2(XP_005258516) c3(heterogeneous nuclear ribonucleoprotein U-like protein I isoform X2) c4(275682) c5(275683) c6( )c7(aw, ei, j, ec); #c1(HNRNPUL1) c2(XP_005258518) c3(heterogeneous nuclear ribonucleoprotein U-like protein I isoform X4) c4(275684) c5( )c6( )c7(aw, ei, j, ec); #c1(HNRNPUL1) c2(XP_005258523) c3(heterogeneous nuclear ribonucleoprotein U-like protein 1 isoform X1) c4( )c5(275685, 275686, 275687, 275688, 275689, 275690) c6( )c7(aw, ei, j, ec); #c1(HNRNPUL1) c2(XP_011524694) c3(heterogeneous nuclear ribonucleoprotein U-like protein I isoform X3) c4( )c5(319179, 319180, 319181, 319182, 319183, 319184, 319185, 319186, 319187, 319188, 319189, 319190, 319191) c6( )c7(aw, ei, j, ec); #c1(HOGA1) c2(NP_001128142) c3(4-hydroxy-2-oxoglutarate aldolase, mitochondrial isoform 2) c4(49628) c5(49629, 49630, 49631, 49632, 49633, 49634) c6( )c7(MB, bpN); #c1(HOGA1) c2(NP_612422) c3(4-hydroxy-2-oxoglutarate aldolase, mitochondrial isoform 1) c4(236428) c5(236429, 236430, 236431, 236432, 236433, 236434) c6( )c7(MB, bpN); #c1(HOMER1) c2(NP_001264006) c3(homer protein homolog I isoform 2) c4(97426) c5(97427, 97428, 97429, 97430, 97431, 97432) c6( )c7(bN, T, bO, Gx, cu, bS, ct, nA, an, Z, FT, Jc); #c1(HOMER1) c2(NP_001264007) c3(homer protein homolog 1 isoform 3) c4( )c5(97433, 97434, 97435, 97436, 97437, 97438) c6( )c7(bN, T, bO, Gx, cu, bS, ct, nA, an, Z, FT, Jc); #c1(HOMER1) c2(NP_004263) c3(homer protein homolog I isoform 1) c4(144697, 144698, 144699) c5(144700, 144701, 144702, 144703, 144704, 144705, 144706, 144707, 144708, 144709, 144710, 144711) c6( )c7(bN, T, b, Gx, cu, bS, ct, nA, an, Z, FT, Jc); #c1(HOMER2) c2(NP_004830) c3(homer protein homolog 2 isoform 1) c4(150118, 150119) c5(150120, 150121, 150122, 150123, 150124, 150125, 150126, 150127, 150128, 150129, 150130, 150131) c6( )c7(ae, T, VV, hV, yk, cT, cu, ci, cm, aed, xg, ad); #c1(HOMER2) c2(NP_955362) c3(homer protein homolog 2 isoform 2) c4(261556, 261557) c5(261558, 261559, 261560, 261561, 261562, 261563, 261564, 261565, 261566, 261567, 261568, 261569) c6( )c7(ae, T, VV, hV, yk, CT, cu, ci, cm, aed, xg, ad); #c1(HOMER3) c2(NP_001139193) c3(homer protein homolog 3 isoform 2) c4(58920) c5(58921, 58922, 58923, 58924, 58925, 58926, 58927, 58928, 58929, 58930, 58931, 58932, 58933, 58934, 58935, 58936) c6( )c7(qK); #c1(HOMER3) c2(NP_001139196) c3(homer protein homolog 3 isoform 3) c4(58937) c5(58938, 58939, 58940, 58941, 58942, 58943, 58944, 58945, 58946, 58947) c6( )c7(qK); #c1(HOMER3) c2(XP_006723007) c3(homer protein homolog 3 isoform X1) c4(294964, 294965) c5(294966, 294967, 294968, 294969, 294970, 294971, 294972, 294973, 294974, 294975, 294976, 294977, 294978, 294979) c6( )c7(qK); #c1(HOMEZ) c2(NP_065885) c3(homeobox and leucine zipper protein Homez) c4(206065, 206066) c5(206067, 206068, 206069, 206070, 206071, 206072, 206073, 206074, 206075, 206076, 206077, 206078) c6( )c7(j); #c1(HOOK2) c2(NP_001093646) c3(protein Hook homolog 2 isoform 2) c4(40413) c5(40414, 40415, 40416, 40417, 40418, 40419) c6( )c7(HQ, T, dH, aW, ht, aT, it, aj, m, hu, t, ae, J, ad); #c1(HOOK2) c2(NP_037444) c3(protein Hook homolog 2 isoform 1) c4(175544) c5(175545, 175546, 175547, 175548, 175549, 175550) c6( )c7(HQ, T, dH, aW, ht, aT, it, aj, m, hu, t, ae, J, ad); #c1 (HOOK3) c2(NP_115786) c3(protein Hook homolog 3) c4(223896) c5(223897, 223898, 223899, 223900, 223901, 223902) c6( ) c7(kl); #c1(HOPX) c2(NP_001138931) c3(homeodomain-only protein isoform b) c4(58282) c5(58283, 58284, 58285, 58286, 58287, 58288, 58289, 58290, 58291, 58292, 58293, 58294, 58295, 58296, 58297, 58298, 58299, 58300, 58301, 58302, 58303, 58304, 58305, 58306, 58307, 58308, 58309, 58310, 58311, 58312, 58313, 58314, 58315, 58316, 58317, 58318, 58319) c6( )c7(T, qb, e, aBx, aA, cz, aj, ci, kO, eA, cd, J, ft, hd, lQ, nJ, d, ff, gm, aF, en, iK, bz, nM, it, iu); #c1(HOPX) c2(NP_001138932) c3(homeodomain-only protein isoform c) c4(58320, 58321) c5(58322, 58323, 58324, 58325, 58326, 58327, 58328, 58329, 58330, 58331, 58332, 58333, 58334, 58335, 58336, 58337, 58338, 58339, 58340, 58341) c6( )c7(T, qb, e, aBx, aA, cz, aj, ci, kO, eA, cd, J, ft, hd, lQ, nJ, d, ff, gm, aF, en, iK, bz, nM, it, iu); #c1(HOPX) c2(NP_115884) c3(homeodomain-only protein isoform a) c4(224479, 224480) c5(224481, 224482, 224483, 224484, 224485, 224486, 224487, 224488, 224489, 224490, 224491, 224492, 224493, 224494, 224495, 224496, 224497, 224498, 224499, 224500, 224501, 224502) c6( )c7(T, qb, e, aBx, aA, cz, aj, ci, kO, eA, cd, J, ft, hd, lQ, nJ, d, ff, gm, aF, en, ik, bz, nM, it, iu); #c1(HORMAD2) c2(NP_689723) c3(HORMA domain-containing protein 2) c4(243668) c5(243669, 243670, 243671, 243672, 243673, 243674, 243675, 243676, 243677, 243678) c6( )c7(fe, bz, ey); #c1(HOXA1) c2(NP_005513) c3(homeobox protein Hox-A1 isoform a) c4(156990) c5(156991, 156992, 156993, 156994, 156995, 156996, 156997) c6( )c7(T, Dh, if, Hn, ci, nA, qN, akW, Mg, iu, anB, ft, aMV, ib, arH, aT, dL, jj, rz, eL, aW, Y, ff, Az, en, g, bz, bN, ck, bKD, bKC, mG, gX, at, bD, xg, B); #c1(HOXA1) c2(NP_705873) c3(homeobox protein Hox-A1 isoform b) c4(246376) c5(246377, 246378, 246379, 246380, 246381, 246382) c6( )c7(T, Dh, if, Hn, ci, nA, qN, akW, Mg, iu, anB, ft, aMV, ib, arH, aT, dL, jj, rz, eL, aW, Y, ff, Az, en, g, bz, bN, ck, bKD, bKC, mG, gX, at, bD, xg, B); #c1 (HOXA10) c2(NP_061824) c3 (homeobox protein Hox-A10) c4(200607, 200608, 200609) c5(200610, 200611, 200612, 200613, 200614, 200615, 200616, 200617, 200618, 200619, 200620, 200621, 200622, 200623, 200624, 200625, 200626, 200627, 200628, 200629) c6( )c7(T, aZ, aA, ci, akW, eA, Vs, ip, bKE, dP, XW, x, aHu, ib, aT, bbu, dz, Ur, yi, d, kl, iU, et, aW, ff, aF, en, mo, PU, t, Px, cl, g, op, ck, bfP, bF, iT, AM, vA, hL, at, anD, np, m, aAl, zl, bt); #c1(HOXA11) c2(NP_005514) c3(homeobox protein Hox-All) c4(156998) c5(156999, 157000, 157001, 157002, 157003, 157004, 157005, 157006, 157007) c6( )c7(T, hF, akW, Vs, bKE, dP, XW, x, aHu, le, Nn, bbu, pq, Ur, d, iU, Y, ff, aoW, en, g, t, bw, bz, atb, ck, eM, bKF, AM, bKG, iT, hL, at, dy, k, m, bt); #c1(HOXA13) c2(XP_011513646) c3(homeobox protein Hox-A13 isoform X1) c4(305945) c5(305946, 305947, 305948, 305949, 305950, 305951, 305952, 305953) c6( )c7(bfP, T, bKH, rV, iU, are, cj, at, aq, it, Pp, aHC, ahl, bKl, iR, bw, bt, akW, B, Ur); #c1(HOXA2) c2(NP_006726) c3(homeobox protein Hox-A2) c4(167464) c5(167465, 167466, 167467, 167468, 167469, 167470, 167471, 167472) c6(167473) c7(XW, ff, aA, en, bKJ); #c1(HOXA3) c2(XP_005249788) c3(homeobox protein Hox-A3 isoform X1) c4(269818, 269819, 269820, 269821) c5(269822, 269823, 269824, 269825, 269826, 269827, 269828, 269829, 269830, 269831, 269832, 269833, 269834, 269835, 269836, 269837, 269838, 269839, 269840, 269841, 269842, 269843, 269844, 269845, 269846, 269847, 269848, 269849, 269850, 269851, 269852, 269853, 269854) c6( )c7(bvL, RX, Az, at, bD, akW, eA, ip); #c1(HOXA4) c2(NP_002132) c3(homeobox protein Hox-A4) c4(125643) c5(125644, 125645, 125646, 125647, 125648, 125649, 125650, 125651, 125652, 125653, (25654) c6( )c7(ck, T, iU, cj, at, Pp, ci, m, g, t, bt, akW); #c1(HOXA5) c2(NP_061975) c3(homeobox protein Hox-A5) c4(201423) c5(201424, 201425, 201426, 201427, 201428, 201429, 201430) c6( )c7(ck, XW, T, akW, jf, eM, iU, aW, vA, wk, iK, ff, aT, at, ci, en, t, bt, bz, op); #c1 (HOXA6) c2(NP_076919) c3(homeobox protein Hox-A6) c4(214237) c5(214238, 214239, 214240, 214241, 214242, 214243) c6( ) c7(at, Bj, T, akW); #c1(HOXA7) c2(NP_008827) c3(homeobox protein Hox-A7) c4(168862, 168863) c5(168864, 168865, 168866, 168867, 168868, 168869, 168870, 168871, 168872, 168873, 168874, 168875) c6( )c7(ck, ib, iT, wu, aW, BJ, ff, aT, at, rO, ci, m, en, g, t, bt, akW, wm, ip); #c1(HOXA9) c2(NP_689952) c3(homeobox protein Hox-A9) c4(244485, 244486, 244487) c5(244488, 244489, 244490, 244491, 244492, 244493, 244494, 244495, 244496, 244497, 244498, 244499, 244500, 244501, 244502, 244503, 244504, 244505) c6( )c7(T, gp, oA, n, oH, ci, hF, akW, eA, br, ad, hX, ip, XW, x, jz, ib, pp, aT, h, cy, dz, K, jj, ae, d, iU, et, aW, Y, ff, en, g, t, bw, bz, pb, ck, eM, iT, dB, cj, at, m, k, bt, kS, B); #c1(HOXB1) c2(NP_002135) c3(homeobox protein Hox-B1) c4(125655) c5(125656, 125657, 125658, 125659, 125660, 125661) c6( )c7(bKM, anB, Ew, aE, iA, pr, bKK, aW, eD, Az, aT, bKL, fr, rz, bt, bN); #c1(HOXB13) c2(NP_006352) c3(homeobox protein Hox-B13) c4(164270) c5(164271, 164272, 164273, 164274, 164275, 164276) c6( )c7(iC, T, bKN, Ub, bE, aW, Y, bFc, hL, aT, r, n, bB, fr, lQ, m, t, ae, q, ad); #c1(HOXB2) c2(NP_002136) c3(homeobox protein Hox-B2) c4(125662, 125663) c5(125664, 125665, 125666, 125667, 125668, 125669, 125670, 125671, 125672, 125673, 125674, 125675) c6( )c7(Ew, aE, iA, pr, aW, eD, aT, dL, fr, jj, g, LV, t, bt, el); #c1(HOXB3) c2(XP_005257339) c3(homeobox protein Hox-B3 isoform X7) c4( )c5(275045, 275046, 275047, 275048, 275049, 275050, 275051, 275052) c6( )c7(ck, iu, Ew, aE, iA, pr, eD, wk, r, fr, bt, q); #c1(HOXB3) c2(XP_011523009) c3(homeobox protein Hox-B3 isoform X2) c4(317493, 317494) c5(317495, 317496) c6( )c7(ck, iu, Ew, aE, iA, pr, eD, wk, r, fr, bt, q); #c1(HOXB3) c2(XP_011523010) c3(homeobox protein Hox-B3 isoform X3) c4( )c5(317497, 317498) c6( )c7(ck, iu, Ew, aE, iA, pr, eD, wk, r, fr, bt, q); #c1(HOXB3) c2(XP_011523022) c3(homeobox protein Hox-B3 isoform X1) c4(317499) c5(317500, 317501, 317502, 317503, 317504, 317505, 317506, 317507, 317508, 317509, 317510, 317511, 317512, 317513, 317514, 317515, 317516, 317517) c6( )c7(ck, iu, Ew, aE, iA, pr, eD, wk, r, fr, bt, q); #c1(HOXB3) c2(XP_011523027) c3(homeobox protein Hox-B3 isoform X8) c4( )c5(317518, 317519) c6( )c7(ck, iu, Ew, aE, iA, pr, eD, wk, r, fr, bt, q); #c1(HOXB3) c2(XP_011523028) c3(homeobox protein Hox-B3 isoform X6) c4( )c5(317520, 317521, 317522, 317523, 317524, 317525, 317526, 317527) c6( )c7(ck, iu, Ew, aE, iA, pr, eD, wk, r, fr, bt, q); #c1(HOXB4) c2(NP_076920) c3(homeobox protein Hox-B4) c4(214244) c5(214245, 214246, 214247, 214248, 214249, 214250) c6( )c7(ck, AK, T, sS, Ew, DY, iA, pr, oV, eD, bt, dW, fr, lQ, hf, mF, V, er); #c1(HOXB5) c2(NP_002138) c3(homeobox protein Hox-B5) c4(125676) c5(125677, 125678, 125679, 125680, 125681, 125682) c6( )c7(T, Ew, iA, pr, eD, v, dL, fr, bt, eL); #c1(HOXB6) c2(XP_005257340) c3(homeobox protein Hox-B6 isoform X1) c4( )c5(275053, 275054, 275055, 275056, 275057, 275058, 275059, 275060) c6( )c7(bv, Ew, iA, pr, eD, r, Pp, fr, t, bt, q); #c1(HOXB6) c2(XP_011523029) c3(homeobox protein Hox-B6 isoform X2) c4(317528) c5(317529, 317530, 317531) c6( )c7(bv, Ew, iA, pr, eD, r, Pp, fr, t, bt, q); #c1(HOXB7) c2(NP_004493) c3(homeobox protein Hox-B7) c4(146903, 146904) c5(146905, 146906, 146907, 146908, 146909, 146910, 146911, 146912, 146913, 146914, 146915, 146916) c6( )c7(nZ, T, if, fd, fr, qN, eJ, q, aE, lt, aT, aEL, d, pr, aW, Y, ff, r, nY, kp, ahl, en, g, ck, Ew, eM, iA, eD, cj, m, bt, iu); #c1(HOXB8) c2(NP_076921) c3(homeobox protein Hox-B8) c4(214251) c5(214252, 214253, 214254, 214255, 214256, 214257) c6( )c7(Ew, iA, pr, eD, fr, bt, qc); #c1(HOXB9) c2(NP_076922) c3(homeobox protein Hox-B9) c4(214258, 214259) c5(214260, 214261, 214262, 214263, 214264, 214265, 214266, 214267) c6( )c7(T, hd, ib, nM, iA, Ew, pr, aW, ev, eD, ex, r, aT, eV, fr, q, bt, B); #c1(HOXB9) c2(XP_011523030) c3(homeobox protein Hox-B9 isoform X1) c4( )c5(317532, 317533, 317534, 317535, 317536) c6( )c7(T, hd, ib, nM, iA, Ew, pr, aW, ev, eD, eX, r, aT, eV, fr, q, bt, B); #c1(HOXC10) c2(NP_059105) c3(homeobox protein Hox-C10) c4(193027) c5(193028, 193029, 193030, 193031, 193032, 193033, 193034) c6( )c7(ck, aW, if, ff, aT, qN, en); #c1(HOXC11) c2(NP_055027) c3(homeobox protein Hox-C11) c4(178367) c5(178368, 178369, 178370, 178371, 178372, 178373, 178374) c6( )c7(ck, T, aW, Y, aT, hn, cm, bt); #c1(HOXC12) c2(NP_776272) c3(homeobox protein Hox-C12) c4(251065) c5(251066, 251067, 251068, 251069, 251070, 251071, 251072) c6( )c7(CN); #c1(HOXC13) c2(NP_059106) c3(homeobox protein Hox-C13) c4(193035) c5(193036, 193037, 193038, 193039, 193040, 193041, 193042) c6( )c7(XW, T, bKO, CN, bt); #c1(HOXC4) c2(NP_705897) c3 (homeobox protein Hox-C4) c4(246383) c5(246384, 246385, 246386, 246387, 246388, 246389, 246390, 246391, 246392, 246393, 246394, 246395) c6( )c7(ck, CN, aE, hk, ks, jj); #c1(HOXC5) c2(NP_061826) c3(homeobox protein Hox-C5) c4(200630) c5(200631, 200632, 200633, 200634, 200635, 200636, 200637) c6( )c7(ck, fM, CN, jh, gZ, hn, Ca); #c1(HOXC6) c2(NP_004494) c3(homeobox protein Hox-CG isoform 1) c4(146917, 146918) c5(146919, 146920, 146921, 146922, 146923, 146924, 146925, 146926, 146927, 146928) c6( )c7(ck, CN, jh, Ca, cm, ae, Yi, ad); #c1(HOXC6) c2(NP_710160) c3(homeobox protein Hox-C6 isoform 2) c4(246524, 246525) c5(246526, 246527, 246528, 246529, 246530, 246531, 246532, 246533, 246534, 246535, 246536, 246537, 246538, 246539, 246540) c6( )c7(ck, CN, jh, Ca, cm, ae, Yi, ad); #c1(HOXC8) c2(NP_073149) c3(homeobox protein Hox-C8) c4(212805) c5(212806, 212807, 212808, 212809, 212810, 212811, 212812, 212813, 212814) c6( )c7(bfP, T, gh, CN, aW, if, aT, n, kp, ci, qN, ae, ad); #c1(HOXC9) c2(NP_008828) c3(homeobox protein Hox-C9) c4(168876) c5(168877, 168878, 168879, 168880, 168881, 168882, 168883, 168884, 168885, 168886, 168887) c6( )c7(cm, aW, CN, aT); #c1(HOXD1) c2(NP_078777) c3(homeobox protein Hox-D1) c4(215425) c5(215426, 215427, 215428, 215429, 215430, 215431, 215432) c6( )c7(t, XW); #c1(HOXD1) c2(XP_005246564) c3 (homeobox protein Hox-D1 isoform X1) c4(267236) c5(267237, 267238, 267239, 267240, 267241, 267242) c6( )c7(t, XW); #c1(HOXD10) c2(NP_002139) c3(homeobox protein Hox-D10) c4(125683) c5(125684, 125685, 125686, 125687, 125688, 125689, 125690) c6( )c7(ael, XW, T, bKP, aW, iK, jh, aT, aA, dL, aF, m, ci, bU, bBe, t, zU, eA, B, y); #c1(HOXD11) c2(NP_067015) c3(homeobox protein Hox-D11) c4(208366, 208367) c5(208368, 208369, 208370, 208371, 208372, 208373, 208374, 208375, 208376, 208377, 208378, 208379, 208380) c6( ) c7(XW, y, gU, ik, aT, dy, aMi, t, bt, atb, bN, bKE); #c1 (HOXD12) c2(NP_067016) c3(homeobox protein Hox-D12) c4(208381) c5(208382, 208383, 208384, 208385, 208386, 208387, 208388) c6( )c7(bN, XW, Y, atb, zU); #c1(HOXD13) c2(NP_000514) c3(homeobox protein Hox-D13) c4(10867) c5(10868, 10869, 10870, 10871, 10872, 10873, 10874) c6( )c7(T, xu, rV, bKR, bry, aHC, zU, bKU, hF, bKT, br, bKS, XW, aE, aT, abj, aOV, af, bKH, lE, iU, aW, bKV, Pp, as, bw, bKQ, atb, bN, hq, ck, arB, bjd, dX, zG, y, bt); #c1(HOXD3) c2(XP_006712540) c3(homeobox protein Hox-D3 isoform X1) c4(286697, 286698) c5(286699, 286700, 286701, 286702, 286703, 286704, 286705, 286706, 286707, 286708, 286709, 286710, 286711, 286712, 286713, 286714, 286715, 286716, 286717, 286718, 286719) c6( )c7(ck, aHF, T, ae, XW, Y, arH, n, j, bz, t, kS, ad); #c1(HOXD4) c2(NP_055436) c3(homeobox protein Hox-D4) c4(181124) c5(181125, 181126, 181127, 181128, 18/129, 181130, 181131) c6( )c7(x, XW, T, eD, hn, k, cm); #c1(HOXD8) c2(NP_001186675) c3(homeobox protein Hox-D8 isoform 2) c4(78315) c5(78316, 78317, 78318, 78319, 78320, 78321, 78322, 78323, 78324, 78325) c6( )c7(cm, XW, A, aHC); #c1(HOXD8) c2(NP_001186676) c3(homeobox protein Hox-D8 isoform 3) c4( )c5(78326, 78327, 78328, 78329, 78330) c6( )c7(cm, XW, A, aHC); #c1(HOXD8) c2(NP_062458) c3(homeobox protein Hox-D8 isoform 1) c4(201802, 201803) c5(201804, 201805, 201806, 201807, 201808, 201809, 201810, 201811) c6( )c7(cm, XW, A, aHC); #c1(HOXD9) c2(NP_055028) c3(homeobox protein Hox-D9) c4(178375) c5(178376, 178377, 178378, 178379, 178380, 178381, 178382) c6( )c7(XW, T, jz, d, dB, Y, if, ff, en, qN, cm, eA, y); #c1(HP) c2(NP_001119574) c3(haptoglobin isoform 2 preproprotein) c4(44148) c5(44149, 44150, 44151, 44152, 44153, 44154, 44155, 44156, 44157, 44158, 44159, 44160) c6( )c7(bM, T, qo, aC, tQ, ea, aUT, e, al, bO, ci, aY, bv, aBb, AW, wM, aE, dV, K, bKW, wt, dQ, eN, aHo, hm, CG, aW, mQ, fc, uY, hJ, uC, yX, vy, QF, wx, m, R, eg, yJ, NK, dD, Ol, aw, yG, bla, n, cQ, aj, fr, ct, qN, ww, cd, ai, b, h, lG, hg, A, mR, fQ, aq, fe, kT, W, cA, mg, te, ER, B, bKX, bL, eR, jx, aA, cR, cz, hy, v, j, eA, J, xx, jZ, aT, yu, bq, an, ae, af, kl, bf, iU, Y, fO, lo, en, ik, pb, vk, ck, BU, gX, agf, mf, cS, dy, fD, aBp, bKY, ao, qb, iW, ha, vr, bS, eq, jE, bKZ, ad, kL, hX, dP, fU, avp, av, F, tT, ds, oW, JK, ji, sj, uX, d, lR, tb, cW, bFM, ff, xs, cr, eZ, kD, yy, aF, hp, Z, qa, wW, D, Cq, gk, qe, yE, aS, i, lf, gq, y); #c1(HP) c2(NP_005134) c3(haptoglobin isoform 1 preproprotein) c4(153293) c5(153294, 153295, 153296, 153297, 153298, 153299, 153300) c6( )c7(bM, T, qo, aC, tQ, ea, aUT, e, al, bO, ci, aY, bv, aBb, AW, wM, aE, dV, K, bKW, wt, dQ, eN, aHo, hm, CG, aW, mQ, fc, uY, hJ, uC, yX, vy, QF, wx, m, R, eg, yJ, NK, dD, Ol, aw, yG, bla, n, cQ, aj, fr, ct, qN, ww, cd, ai, b, h, lG, hg, A, mR, fQ, aq, fe, kT, W, cA, mg, te, ER, B, bKX, bL, eR, jx, aA, CR, cz, hy, v, j, eA, J, xx, jZ, aT, yu, bq, an, ae, af, kl, bf, iU, Y, fO, lo, en, iK, pb, vk, ck, BU, gX, agf, mf, cS, dy, fD, aBp, bKY, ao, qb, iW, ha, vr, bS, eq, jE, bKZ, ad, kL, hX, dP, fU, avp, av, F, tT, ds, oW, JK, ji, sj, uX, d, lR, tb, cW, bFM, ff, xs, cr, eZ, kD, yy, aF, hp, Z, qa, wW, D, Cq, gk, qe, yE, aS, i, lf, gq, y); #c1(HP1BP3) c2(XP_011539836) c3(heterochromatin protein 1-binding protein 3 isoform X2) c4(338074, 338075) c5(338076, 338077, 338078, 338079, 338080, 338081, 338082, 338083, 338084, 338085, 338086, 338087, 338088, 338089, 338090, 338091, 338092) c6( )c7(aqN); #c1(HP1BP3) c2(XP_011539837) c3(heterochromatin protein 1-binding protein 3 isoform X3) c4(338093, 338094, 338095, 338096, 338097) c5(338098, 338099, 338100, 338101, 338102, 338103, 338104, 338105, 338106, 338107, 338108, 338109, 338110, 338111, 338112, 338113, 338114, 338115, 338116, 338117, 338118, 338119, 338120, 338121, 338122, 338123, 338124, 338125, 338126, 338127, 338128, 338129, 338130, 338131, 338132, 338133, 338134, 338135, 338136, 338137, 338138, 338139) c6( )c7(aqN); #c1(HPCAL1) c2(XP_011508650) c3(hippocalcin-like protein I isoform X1) c4(299259, 299260) c5(299261, 299262, 299263, 299264, 299265, 299266, 299267, 299268, 299269, 299270, 299271) c6( )c7(cm, T, cz, pN); #c1(HPCAL1) c2(XP_011508651) c3(hippocalcin-like protein I isoform X2) c4( )c5(299272, 299273, 299274, 299275, 299276, 299277) c6( )c7(cm, T, cz, pN); #c1(HPD) c2(NP_001165464) c3(4-hydroxyphenylpyruvate dioxygenase isoform 2) c4( )c5(67752, 67753, 67754, 67755, 67756) c6( )c7(gh, T, e, al, zU, Ll, ad, LH, aE, ec, ds, blb, aU, vw, LG, ae, bLc, aq, LJ, iR, CQ, ig, QT, dy, zG); #c1(HPD) c2(NP_002141) c3(4-hydroxyphenylpyruvate dioxygenase isoform 1) c4(125691, 125692, 125693) c5(125694, 125695, 125696, 125697, 125698, 125699, 125700, 125701, 125702, 125703, 125704, 125705, 125706, 125707, 125708, 125709, 125710, 125711, 125712, 125713, 125714, 125715, 125716, 125717, 125718, 125719, 125720, 125721, 125722, 125723, 125724, 125725, 125726, 125727, 125728, 125729, 125730, 125731, 125732, 125733, 125734, 125735, 125736, 125737, 125738, 125739, 125740, 125741, 125742, 125743, 125744, 125745, 125746, 125747, 125748, 125749, 125750, 125751, 125752, 125753, 125754, 125755, 125756, 125757, 125758, 125759, 125760, 125761, 125762, 125763, 125764, 125765, 125766, 125767, 125768, 125769, 125770, 125771, 125772, 125773, 125774, 125775, 125776, 125777, 125778, 125779, 125780, 125781, 125782, 125783, 125784, 125785, 125786, 125787, 125788, 125789, 125790, 125791, 125792, 125793) c6(125794) c7(gh, T, e, al, zU, Ll, ad, LH, aE, ec, ds, blb, aU, vw, LG, ae, blc, aq, LJ, iR, cA, ig, QT, dy, zG); #c1(HPGD) c2(NP_000851) c3(15-hydroxyprostaglandin dehydrogenase [NAD(+)] isoform 1) c4(16579, 16580) c5(16581, 16582, 16583, 16584, 16585, 16586, 16587, 16588) c6( )c7(sY, T, dA, aA, bB, bLd, ci, Ea, q, ad, dP, av, bLe, jz, aT, af, d, B, s, aW, aBN, aBM, r, aq, Au, aF, g, avG, jT, uC, bz, bF, c, nB, bE, gX, pg, lC, X); #c1(HPGD) c2(NP_001139288) c3(15-hydroxyprostaglandin dehydrogenase [NAD(+)] isoform 2) c4(59130) c5(59131, 59132, 59133, 59134, 59135, 59136) c6( )c7(sY, T, dA, aA, bB, bLd, ci, Ea, q, ad, dP, av, bLe, jz, aT, af, d, B, s, aW, aBN, aBM, r, aq, Au, aF, g, avG, jT, uC, bz, bF, c, nB, bE, gX, pg, lC, X); #c1(HPGD) c2(NP_001243234) c3(15-hydroxyprostaglandin dehydrogenase [NAD(+)] isoform 4) c4(90209) c5(90210, 90211, 90212, 90213, 90214, 90215) c6( )c7(sY, T, dA, aA, bB, bLd, ci, Ea, q, ad, dP, av, bLe, jz, aT, af, d, B, s, aW, aBN, aBM, r, aq, Au, aF, g, avG, jT, uC, bz, bF, c, nB, bE, gX, pg, lC, X); #c1(HPGD) c2(NP_001243236) c3(15-hydroxyprostaglandin dehydrogenase [NAD(+)] isoform 3) c4( )c5(90216, 90217, 90218, 90219, 90220, 90221, 90222, 90223) c6( )c7(sY, T, dA, aA, bB, bLd, ci, Ea, q, ad, dP, av, bLe, jz, aT, af, d, B, s, aW, aBN, aBM, r, aq, Au, aF, g, avG, jT, uC, bz, bF, c, nB, bE, gX, pg, lC, X); #c1(HPGDS) c2(NP_055300) c3(hematopoietic prostaglandin D synthase) c4(180371) c5(180372, 180373, 180374, 180375, 180376, 180377) c6( )c7(bM, T, pf, ea, if, e, al, eb, bLf, bB, fd, qj, ci, bh, Jx, bLg, ip, x, Gt, aE, ja, K, hu, hH, pk, dF, bc, Zm, hm, Qe, aa, eL, dH, Jl, aW, iE, az, hl, CT, uY, Au, bBH, g, as, eE, OR, lk, eF, cm, bw, yd, bb, xK, eM, nB, iA, iT, go, vA, cj, uJ, dW, bcl, k, Hb, m, dD, gU, aWG, ht, NT, aw, n, aj, fr, qN, pM, fK, br, co, b, ft, ON, eT, gd, h, jy, eV, hn, dL, hB, he, Um, dx, OV, A, sp, hQ, fe, aq, bFN, pm, dw, Ld, op, asE, W, bP, dB, hk, Qu, at, fL, YH, it, B, L, bL, eR, AL, aZy, yk, jx, eX, aA, GP, cz, fF, v, j, Qo, AO, V, q, J, er, eA, XX, akU, aoA, yg, aT, vH, yu, bK, aKG, an, ae, af, bA, jf, qw, s, ND, vT, iU, Y, Lf, r, en, bu, cl, t, f, ik, pb, bN, ck, cT, sS, tW, c, ac, Nk, gX, fD, bt, rA, ao, blu, gx, id, aDy, qb, aD, iW, rl, bs, lD, jh, oZ, Fb, Jz, fu, Gn, eq, iG, hF, pl, mC, ad, hX, dP, hd, F, py, io, ji, d, qt, bFM, ff, xs, eH, aZw, rM, dN, Ej, cr, aF, eZ, S, iV, bz, qa, ek, bF, es, gk, apM, bE, z, gZ, i, hE, iu, y, pC); #c1(HPN) c2(NP_892028) c3(serine protease hepsin precursor) c4(257667) c5(257668, 257669, 257670, 257671, 257672, 257673, 257674, 257675, 257676, 257677, 257678, 257679, 257680, 257681) c6( )c7(T, ad, bm, aq, Fb, PQ, ci, m, du, fr, t, ae, B, us, pl, hX); #c1(HPN) c2(XP_011525193) c3(serine protease hepsin isoform X2) c4(319668) c5(319669, 319670, 319671, 319672, 319673, 319674) c6( )c7(T, ad, bm, aq, Fb, PQ, ci, m, du, fr, t, ae, B, us, pl, hX); #c1(HPR) c2(NP_066275) c3(haptoglobin-related protein precursor) c4(206905, 206906) c5(206907, 206908, 206909, 206910, 206911, 206912, 206913, 206914, 206915, 206916, 206917, 206918, 206919, 206920, 206921, 206922) c6( )c7(T, eA, ad, x, ft, dV, aT, dL, K, bKW, XZ, ae, eL, CG, d, aW, blh, aq, cl, t, cm, ck, FV, hL, m, k, bt, B); #c1(HPRT1) c2(NP_000185) c3(hypoxanthine-guanine phosphoribosyltransferase) c4(4011, 4012, 4013) c5(4014, 4015, 4016, 4017, 4018, 4019, 4020, 4021, 4022, 4023, 4024, 4025, 4026, 4027, 4028, 4029, 4030, 4031, 4032, 4033, 4034, 4035, 4036, 4037, 4038, 4039, 4040, 4041, 4042, 4043, 4044, 4045, 4046, 4047, 4048, 4049, 4050, 4051, 4052, 4053, 4054, 4055, 4056, 4057, 4058, 4059, 4060, 4061, 4062, 4063, 4064, 4065, 4066, 4067, 4068, 4069, 4070, 4071, 4072, 4073, 4074, 4075, 4076, 4077, 4078, 4079, 4080, 4081, 4082, 4083, 4084, 4085, 4086, 4087, 4088, 4089, 4090, 4091, 4092, 4093, 4094, 4095, 4096, 4097, 4098, 4099, 4100, 4101, 4102, 4103, 4104, 4105, 4106, 4107, 4108, 4109, 4110, 4111, 4112, 4113, 4114, 4115, 4116, 4117) c6(4118, 4119, 4120, 4121, 4122) c7(nD, agx, os, T, aYP, jh, fg, bB, KO, oZ, OY, ci, agz, mF, hF, V, aR, ad, Oe, x, av, Mo, oT, nH, fh, dV, aT, dz, hn, adV, K, bq, bLj, jj, amA, Ha, ae, CA, A, aQp, hG, RO, aW, Y, zb, lb, CT, fe, uY, aq, cm, DL, bz, dr, pb, ck, mO, Bv, Dy, eM, yW, aYa, bE, X, HK, bLi, uo, Hs, eo, k, m, bC, y, kS, B, jr); #c1 (HPRT1) c2(XP_011529630) c3(hypoxanthine-guanine phosphoribosyltransferase isoform X1) c4(325157) c5( )c6( )c7(nD, agx, os, T, aYP, jh, fg, bB, KO, oZ, OY, ci, agz, mF, hF, V, aR, ad, Oe, x, av, Mo, oT, nH, fh, dV, aT, dz, hn, adV, K, bq, bLj, jj, amA, Ha, ae, CA, A, aQp, hG, RO, aW, Y, zb, lb, CT, fe, uY, aq, cm, DL, bz, dr, pb, ck, mO, Bv, Dy, eM, yW, aYa, bE, X, HK, bLi, uo, Hs, eo, k, m, bC, y, kS, B, jr); #c1(HPS1) c2(NP_000186) c3(Hermansky-Pudlak syndrome I protein isoform a) c4(4123) c5(4124, 4125, 4126, 4127, 4128, 4129, 4130, 4131, 4132, 4133) c6( )c7(fG, e, aw, jN, j, q, XB, Tl, XD, bW, ji, af, eu, lE, Y, fc, XC, r, aq, Xz, Xy, KM, ac, hk, XF, lF, dX, cP, B); #c1(HPS1) c2(NP_872577) c3(Hermansky-Pudlak syndrome 1 protein isoform c) c4(256390, 256391) c5(256392, 256393, 256394, 256395, 256396, 256397, 256398, 256399, 256400, 256401, 256402, 256403) c6( )c7(fG, e, aw, jN, j, q, XB, Tl, XD, bW, ji, af, eu, lE, Y, fc, XC, r, aq, Xz, Xy, KM, ac, hk, XF, lF, dX, cP, B); #c1(HPS1) c2(XP_005269815) c3(Hermansky-Pudlak syndrome I protein isoform X2) c4(282877, 282878) c5(282879, 282880, 282881, 282882, 282883, 282884, 282885, 282886, 282887, 282888, 282889, 282890) c6( )c7(fG, e, aw, jN, j, q, XB, Tl, XD, bW, ji, af, eu, lE, Y, fc, XC, r, aq, Xz, Xy, KM, ac, hk, XF, lF, dX, cP, B); #c1(HPS3) c2(NP_115759) c3(Hermansky-Pudlak syndrome 3 protein) c4(223845) c5(223846, 223847, 223848, 223849, 223850, 223851) c6( )c7(XF, lE, bLk, lF); #c1(HPS4) c2(NP_071364) c3(Hermansky-Pudlak syndrome 4 protein isoform a) c4(211212) c5(211213, 211214, 211215, 211216, 211217, 211218, 211219, 211220, 211221, 211222) c6( )c7(lE, bW, fG, XF, lF, dX, ji, bLl); #c1(HPS4) c2(NP_690054) c3(Hermansky-Pudlak syndrome 4 protein isoform b) c4(244849) c5(244850, 244851, 244852, 244853, 244854, 244855) c6( )c7(lE, bW, fG, XF, lF, dX, ji, bLl); #c1(HPS4) c2(XP_006724417) c3(Hermansky-Pudlak syndrome 4 protein isoform X3) c4(295954, 295955) c5(295956, 295957, 295958, 295959, 295960, 295961, 295962, 295963, 295964, 295965, 295966, 295967) c6( )c7(lE, bW, fG, XF, lF, dX, ji, bLl); #c1(HPS5) c2(NP_852608) c3(Hermansky-Pudlak syndrome 5 protein isoform a) c4(255055) c5(255056, 255057, 255058, 255059, 255060, 255061, 255062, 255063) c6( )c7(lE, XF, bLm); #c1(HPS5) c2(NP_852609) c3(Hermansky-Pudlak syndrome 5 protein isoform b) c4(255064) c5(255065, 255066, 255067, 255068, 255069, 255070, 255071, 255072) c6( )c7(lE, XF, bLm); #c1(HPS5) c2(XP_011518166) c3(Hermansky-Pudlak syndrome 5 protein isoform X1) c4( )c5(312201) c6( )c7(lE, XF, bLm); #c1(HPS6) c2(NP_079023) c3(Hermansky-Pudlak syndrome 6 protein) c4(216628) c5(216629, 216630, 216631, 216632, 216633, 216634, 216635, 216636, 216637, 216638) c6( )c7(bLn, XF); #c1(HPSE) c2(NP_001092010) c3(heparanase isoform 1 preproprotein) c4(39248, 39249) c5(39250, 39251, 39252, 39253, 39254, 39255, 39256, 39257, 39258, 39259) c6( )c7(T, aC, Zz, ea, if, fd, bB, ci, x, bv, aE, pX, K, hu, wt, DY, eL, Us, cp, Qe, hm, eG, aW, aEl, apn, g, as, eF, cm, bw, eM, nB, k, Hb, m, dD, pA, sw, aw, ht, fN, qy, n, aj, fr, cd, Vs, jz, Ru, blp, h, hn, dL, ey, A, hQ, fQ, aq, fe, gG, cM, iO, dB, vb, hl, bm, hR, va, bLq, it, B, bL, AS, dA, dk, lH, aA, hy, v, j, C, q, J, eA, eJ, aT, ae, af, bA, jf, arl, Qr, NE, nO, oV, Y, ev, iU, r, en, t, ik, ck, ac, gX, cS, dy, im, bt, rA, hS, qb, bLo, eq, uu, ah, Gn, hF, Fc, ad, hX, dP, av, hd, Db, yt, nJ, d, lR, qt, ff, eH, cr, dN, Ej, aF, iR, bz, bE, aS, rH, y, iu); #c1(HPSE) c2(NP_001159970) c3(heparanase isoform 2 preproprotein) c4(65007) c5(65008, 65009, 65010, 65011, 65012, 65013) c6( )c7(T, aC, Zz, ea, if, fd, bB, ci, x, bv, aE, pX, K, hu, wt, DY, eL, Us, cp, Qe, hm, eG, aW, aEl, apn, g, as, eF, cm, bw, eM, nB, k, Hb, m, dD, pA, sw, aw, ht, fN, qy, n, aj, fr, cd, Vs, jz, Ru, bLp, h, hn, dL, ey, A, hQ, fQ, aq, fe, gG, cM, iO, dB, vb, hL, bm, hR, va, blq, it, B, bL, AS, dA, dK, lH, aA, hy, v, j, C, q, J, eA, eJ, aT, ae, af, bA, jf, arl, Qr, NE, nO, oV, Y, ev, iU, r, en, t, iK, ck, ac, gX, cS, dy, im, bt, rA, hS, qb, bLo, eq, uu, ah, Gn, hF, Fc, ad, hX, dP, av, hd, Db, yt, nJ, d, lR, qt, ff, eH, cr, dN, Ej, aF, iR, bz, bE, aS, rH, y, iu); #c1(HPSE) c2(NP_001186759) c3(heparanase isoform 3 preproprotein) c4(78523) c5(78524, 78525, 78526, 78527, 78528, 78529, 78530, 78531, 78532, 78533, 78534, 78535) c6( )c7(T, aC, Zz, ea, if, fd, bB, ci, x, bv, aE, pX, K, hu, wt, DY, eL, Us, cp, Qe, hm, eG, aW, aEl, apn, g, as, eF, cm, bw, eM, nB, k, Hb, m, dD, pA, sw, aw, ht, fN, qy, n, aj, fr, cd, Vs, jz, Ru, bLp, h, hn, dL, ey, A, hQ, fQ, aq, fe, gG, cM, iO, dB, vb, hL, bm, hR, va, bLq, it, B, bL, AS, dA, dK, lH, aA, hy, v, j, C, q, J, eA, eJ, aT, ae, af, bA, jf, arl, Qr, NE, nO, oV, Y, ev, iU, r, en, t, ik, ck, ac, gX, cS, dy, im, bt, rA, hS, qb, bLo, eq, uu, ah, Gn, hF, Fc, ad, hX, dP, av, hd, Db, yt, nJ, d, lR, qt, ff, eH, cr, dN, Ej, af, iR, bz, bE, aS, rH, y, iu); #c1(HPSE2) c2(NP_001159716) c3 (inactive heparanase-2 isoform 2) c4(64609) c5(64610, 64611, 64612, 64613, 64614, 64615) c6( )c7(T, aw, fd, n, aj, j, ci, uu, ad, J, x, qh, wt, or, hm, arl, ZH, CN, ff, en, bEv, iK, ac, avS, vb, mg, cj, bfX, bt); #c1(HPSE2) c2(NP_001159717) c3(inactive heparanase-2 isoform 3) c4(64616) c5(64617, 64618, 64619, 64620, 64621, 64622) c6( )c7(T, aw, fd, n, aj, j, ci, uu, ad, J, x, qh, wt, or, hm, arl, ZH, CN, ff, en, bEv, iK, ac, avS, vb, mg, cj, bfX, bt); #c1(HPSE2) c2(NP_001159718) c3(inactive heparanase-2 isoform 4) c4(64623) c5(64624, 64625, 64626, 64627, 64628, 64629) c6( )c7(T, aw, fd, n, aj, j, ci, uu, ad, J, x, qh, wt, or, hm, arl, ZH, CN, ff, en, bEv, iK, ac, avS, vb, mg, cj, bfX, bt); #c1(HPSE2) c2(NP_068600) c3(inactive heparanase-2 isoform 1) c4(209768) c5(209769, 209770, 209771, 209772, 209773, 209774) c6( )c7(T, aw, fd, n, aj, j, ci, uu, ad, J, x, qh, wt, or, hm, arl, ZH, CN, ff, en, bEv, iK, ac, avS, vb, mg, cj, bfX, bt); #c1(HPX) c2(XP_005252940) c3(hemopexin isoform X1) c4(272074, 272075) c5(272076, 272077, 272078, 272079, 272080, 272081, 272082, 272083, 272084, 272085, 272086, 272087) c6( )c7(md, bL, ai, bLr, uL, bLs, aW, cj, bjL, zo, uY, aq, ah, eg, Ud, cM, B, ad); #c1(HR) c2(NP_005135) c3(lysine-specific demethylase hairless isoform a) c4(153301) c5(153302, 153303, 153304, 153305, 153306, 153307, 153308) c6( )c7(bLt, bbW, akR, bLv, bLu, bwS, bwT); #c1(HR) c2(NP_060881) c3(lysine-specific demethylase hairless isoform b) c4(198313) c5(198314, 198315, 198316, 198317, 198318, 198319) c6( ) c7(bLt, bbW, akR, bLv, bLu, bwS, bwT); #c1(HRAS) c2(NP_001123914) c3(GTPase HRas isoform 1) c4(47996) c5(47997, 47998, 47999, 48000, 48001, 48002, 48003, 48004, 48005, 48006, 48007, 48008, 48009, 48010) c6( )c7(bLy, T, aC, fR, azf, hV, yS, if, fd, bB, ci, aon, aJM, bv, afr, aE, rJ, iv, Ou, auF, K, hu, eL, Zm, eG, zQ, aW, CT, Qv, Qq, g, as, eF, zE, cm, apB, bw, qJ, eM, nB, vA, vy, azg, m, yA, sc, QO, wu, gp, n, ko, kv, nA, qN, zP, br, co, aBy, b, Bl, ft, i, aMD, KN, Ak, aqK, jy, dL, A, ks, aq, op, ar, Qy, bP, dB, hk, Qu, Vy, hL, aMl, B, cn, bL, AS, dA, jp, dK, yk, WT, jx, fZ, aA, v, pd, aYD, q, er, eA, xx, lg, nv, Vr, qK, aT, bK, bq, ae, ne, jf, s, vT, Qr, oV, Y, ev, bLA, iU, r, aMA, aJl, lo, en, bu, cl, t, bkp, iK, pb, bN, zJ, ov, ck, sS, Dy, gX, WU, aor, bsT, im, bt, bLx, id, pY, aD, ml, jh, bs, bLz, hF, ad, av, hd, aop, Db, NS, oj, hD, wm, mu, d, hG, ff, Mz, aF, iV, iR, bz, dr, wW, RV, Ew, bE, pP, Km, adg, blw, X); #c1(HRAS) c2(NP_789765) c3(GTPase HRas isoform 2) c4(252211) c5(252212, 252213, 252214, 252215, 252216, 252217, 252218, 252219, 252220, 252221, 252222, 252223, 252224, 252225, 252226, 252227, 252228, 252229, 252230, 252231) c6( )c7(bLy, T, aC, fR, azf, hV, yS, if, fd, bB, ci, aon, aJM, bv, afr, aE, rJ, iv, Ou, auf, K, hu, eL, Zm, eG, zQ, aW, cT, Qv, Qq, g, as, eF, zE, cm, apB, bw, qJ, eM, nB, vA, vy, azg, m, yA, sc, QO, wu, gp, n, ko, kv, nA, qN, zP, br, co, aBy, b, Bl, ft, i, aMD, KN, Ak, aqK, jy, dL, A, ks, aq, op, ar, Qy, bP, dB, hk, Qu, Vy, hL, aMl, B, cn, bL, AS, dA, jp, dk, yk, WT, jx, fZ, aA, v, pd, aYD, q, er, eA, xx, lg, nv, Vr, qK, aT, bK, bq, ae, ne, jf, s, vT, Qr, oV, Y, ev, bLA, iU, r, aMA, aJl, lo, en, bu, cl, t, bkp, ik, pb, bN, zJ, ov, ck, sS, Dy, gX, WU, aor, bsT, im, bt, bLx, id, pY, aD, ml, jh, bs, bLz, hF, ad, av, hd, aop, Db, NS, oj, hD, wm, mu, d, hG, ff, Mz, aF, iV, iR, bz, dr, wW, RV, Ew, bE, pP, Km, adg, bLw, X); #c1(HRASLS) c2(NP_065119) c3(phospholipid-metabolizing enzyme A-C1) c4(204039) c5(204040, 204041, 204042, 204043, 204044, 204045) c6( )c7(dD, aV, ec, cW, ea, az, aA, cz, j, eq, Sm, ae, ad); #c1(HRC) c2(NP_002143) c3(sarcoplasmic reticulum histidine-rich calcium-binding protein precursor) c4(125795, 125796, 125797) c5(125798, 125799, 125800, 125801, 125802, 125803, 125804, 125805, 125806, 125807, 125808, 125809, 125810, 125811, 125812, 125813, 125814, 125815) c6( )c7(bL, es, iA, mx, aj, K, eA, mo, nN, J, cd, dr); #c1(HRG) c2(NP_000403) c3(histidine-rich glycoprotein precursor) c4(8552) c5(8553, 8554, 8555, 8556, 8557, 8558, 8559, 8560, 8561, 8562, 8563) c6( )c7(oO, T, aw, C, eA, aE, vh, aT, bLB, K, bLC, d, aW, sq, ff, en, DL, Qt, dr, dB, se, ag); #c1(HRG) c2(XP_005247472) c3(histidine-rich glycoprotein isoform X1) c4( )c5(267851) c6( )c7(oO, T, aw, C, eA, aE, vh, aT, bLB, K, bLC, d, aW, sq, ff, en, DL, Qt, dr, dB, se, ag); #c1(HRH1) c2(NP_001091683) c3(histamine H1 receptor) c4(38704) c5(38705, 38706, 38707, 38708, 38709, 38710, 38711, 38712, 38713, 38714, 38715, 38716, 38717, 38718, 38719, 38720, 38721) c6( )c7(bM, cT, dD, ac, ea, Jp, e, JM, aj, j, eq, an, aOJ, X, J); #c1(HRH2) c2(NP_001124527) c3(histamine H2 receptor isoform 1) c4(49186) c5(49187, 49188, 49189, 49190, 49191, 49192) c6( )c7(ov, id, aJt, ea, Y, atj, az, aA, vs, aB, aF, Qo, aOJ, eN, X); #c1(HRH2) c2(NP_071640) c3(histamine H2 receptor isoform 2) c4( )c5(211798, 211799, 211800, 211801, 211802, 211803) c6( )c7(ov, id, aJt, ea, Y, atj, az, aA, vs, aB, aF, Qo, aOJ, eN, X); #c1(HRH2) c2(XP_006714928) c3(histamine H2 receptor isoform X2) c4(288364) c5( )c6( )c7(ov, id, alt, ea, Y, atj, az, aA, vs, aB, aF, Qo, aOJ, eN, X); #c1(HRH3) c2(NP_009163) c3(histamine H3 receptor) c4( )c5(171206, 171207, 171208, 171209, 171210, 171211, 171212, 171213, 171214, 171215, 171216, 171217) c6( )c7(ke, bM, cF, ot, Bc, dJ, My, Nt, cz, lG, GC, dG, v, ci, cB, dl); #c1(HRH3) c2(XP_005260323) c3(histamine H3 receptor isoform X1) c4(276514, 276515, 276516) c5(276517, 276518, 276519, 276520, 276521, 276522) c6( )c7(ke, bM, cF, ot, Bc, dJ, My, Nt, cz, lG, GC, dG, v, ci, cB, dl); #c1(HRH3) c2(XP_005260324) c3(histamine H3 receptor isoform X2) c4( )c5(276523, 276524) c6( )c7(ke, bM, cF, ot, Bc, dJ, My, Nt, cz, lG, GC, dG, v, ci, cB, dl); #c1(HRH4) c2(NP_001137300) c3(histamine H4 receptor isoform 2) c4(55380) c5(55381, 55382, 55383, 55384, 55385, 55386) c6( )c7(ai, eE, cr, aA, r, eZ, aF, ci, g, aOJ, y, X, dr); #c1(HRH4) c2(NP_067637) c3(histamine H4 receptor isoform 1) c4(209134) c5(209135, 209136, 209137, 209138, 209139, 209140) c6( )c7(ai, eE, cr, aA, r, eZ, aF, ci, g, aOJ, y, X, dr); #c1(HRK) c2(NP_003797) c3(activator of apoptosis harakiri) c4(140957) c5(140958, 140959, 140960, 140961, 140962, 140963) c6( )c7(ck, T, d, iW, jh, r, aA, af, as, ij, ae, ad); #c1(HRNR) c2(NP_001009931) c3(hornerin) c4(23735) c5(23736, 23737, 23738, 23739, 23740, 23741, 23742, 23743) c6( )c7(T, aW, aT, eE, AO, eA); #c1(HRSP12) c2(NP_005827) c3(ribonuclease UK114) c4(159720) c5(159721, 159722, 159723, 159724, 159725, 159726) c6( )c7(ar, dt, jp, fQ, kp, act, ae, ad); #c1(HS1BP3) c2(NP_071905) c3(HCLS1-binding protein 3) c4(212212) c5(212213, 212214, 212215, 212216, 212217, 212218) c6( )c7(Gt, aMV, byQ, an, bLD, xw); #c1(HS3ST1) c2(NP_005105) c3(heparan sulfate glucosamine 3-O-sulfotransferase I precursor) c4(152921, 152922) c5(152923, 152924, 152925, 152926, 152927, 152928, 152929, 152930, 152931, 152932, 152933, 152934, 152935, 152936, 152937, 152938, 152939, 152940) c6(152941, 152942) c7(T, aE, aw, cz, eW, bA); #c1(HS3ST2) c2(NP_006034) c3(heparan sulfate glucosamine 3-O-sulfotransferase 2) c4(161322, 161323) c5(161324, 161325, 161326, 161327, 161328, 161329, 161330, 161331, 161332, 161333, 161334, 161335) c6( )c7(T, QO, fR, eM, aW, if, ff, jh, r, aT, en, qN, bt, bz, q); #c1(HS3ST3A1) c2(NP_006033) c3(heparan sulfate glucosamine 3-O-sulfotransferase 3A1) c4(161315) c5(161316, 161317, 161318, 161319, 161320, 161321) c6( )c7(Gp, oe, eW, eZ, c); #c1(HS3ST3B1) c2(NP_006032) c3(heparan sulfate glucosamine 3-O-sulfotransferase 3B1) c4(161304) c5(161305, 161306, 161307, 161308, 161309, 161310, 161311, 161312, 161313, 161314) c6( )c7(dP, lo, aE, hl); #c1(HS3ST4) c2(NP_006031) c3(heparan sulfate glucosamine 3-O-sulfotransferase 4) c4(161297) c5(161298, 161299, 161300, 161301, 161302, 161303) c6( )c7(cd, xa, ac, bM); #c1(HS3ST5) c2(NP_705840) c3(heparan sulfate glucosamine 3-O-sulfotransferase 5) c4(246326) c5(246327, 246328, 246329, 246330, 246331, 246332, 246333, 246334, 246335, 246336) c6( )c7(Y); #c1(HS3ST6) c2(NP_001009606) c3(heparan sulfate glucosamine 3-O-sulfotransferase 6) c4(23652, 23653, 23654) c5(23655, 23656, 23657, 23658, 23659, 23660, 23661, 23662, 23663, 23664, 23665, 23666, 23667, 23668, 23669, 23670, 23671, 23672) c6( )c7(eW); #c1(HSGST1) c2(NP_004798) c3(heparan-sulfate 6-O-sulfotransferase 1) c4(149935) c5(149936, 149937, 149938, 149939, 149940, 149941) c6( )c7(LB, bLE); #c1(HS6ST2) c2(NP_001070656) c3(heparan-sulfate 6-O-sulfotransferase 2 isoform L) c4(34652, 34653) c5(34654, 34655, 34656, 34657, 34658, 34659) c6( )c7(LB, aW, oq, aT); #c1(HS6ST2) c2(NP_671704) c3(heparan-sulfate 6-O-sulfotransferase 2 isoform S) c4(242081, 242082) c5(242083, 242084, 242085, 242086, 242087, 242088, 242089, 242090, 242091, 242092, 242093, 242094) c6( ) c7(LB, aW, oq, aT); #c1(HS6ST2) c2(XP_005262548) c3(heparan-sulfate 6-O-sulfotransferase 2 isoform X2) c4(278006) c5(278007, 278008, 278009, 278010, 278011, 278012) c6( )c7(LB, aW, oq, aT); #c1(HS6ST2) c2(XP_011529708) c3(heparan-sulfate 6-O-sulfotransferase 2 isoform X3) c4(325335) c5(325336, 325337, 325338, 325339, 325340, 325341, 325342, 325343, 325344, 325345, 325346, 325347) c6( )c7(LB, aW, oq, aT); #c1(HS6ST3) c2(NP_703157) c3(heparan-sulfate 6-O-sulfotransferase 3 precursor) c4(246076) c5(246077, 246078, 246079, 246080, 246081, 246082) c6( )c7(vy, Wb, aj, v, J, sj); #c1(HS6ST3) c2(XP_011519378) c3(heparan-sulfate 6-O-sulfotransferase 3 isoform X6) c4( )c5(314046, 314047, 314048) c6( )c7(vy, Wb, aj, v, J, sj); #c1(HSBP1) c2(NP_001528) c3(heat shock factor-binding protein 1) c4(118618) c5(118619, 118620, 118621, 118622, 118623, 118624) c6( )c7(Hy, K, aT); #c1(HSD11B1) c2(NP_001193670) c3(corticosteroid 11-beta-dehydrogenase isozyme 1) c4(82444) c5(82445, 82446, 82447, 82448, 82449, 82450, 82451, 82452, 82453, 82454, 82455, 82456, 82457, 82458) c6( )c7(bL, T, bLH, bLl, eR, fR, sQ, aZ, vf, e, cz, bS, j, ct, dG, ml, v, OH, q, tg, aT, vg, K, lQ, bq, uV, bLG, dQ, dF, af, qM, bA, aRR, hK, kl, s, cW, aW, fJ, eH, r, dq, Hu, aUO, Z, eF, bN, bJ, bbT, bLF, uo, hR, ut, mf, ei, arg, aOo); #c1(HSD11B1L) c2(NP_001254797) c3(hydroxysteroid 11-beta-dehydrogenase 1-like protein isoform g) c4( )c5(94956, 94957, 94958, 94959, 94960) c6( )c7(ae, ci, ad); #c1(HSD11B1L) c2(NP_001254798) c3(hydroxysteroid 11-beta-dehydrogenase 1-like protein isoform d) c4( )c5(94961, 94962, 94963, 94964, 94965, 94966, 94967) c6(94968, 94969) c7(ae, ci, ad); #c1(HSD11B1L) c2(NP_001254800) c3(hydroxysteroid 11-beta-dehydrogenase 1-like protein isoform i precursor) c4(94970) c5(94971, 94972, 94973, 94974, 94975, 94976, 94977, 94978, 94979) c6(94980, 94981) c7(ae, ci, ad); #c1(HSD11B1L) c2(NP_940935) c3(hydroxysteroid 11-beta-dehydrogenase 1-like protein isoform e precursor) c4(260137, 260138, 260139, 260140) c5(260141, 260142, 260143, 260144, 260145, 260146, 260147, 260148) c6(260149, 260150) c7(ae, ci, ad); #c1(HSD11B1L) c2(NP_941993) c3(hydroxysteroid 11-beta-dehydrogenase 1-like protein isoform f) c4( )c5(260342, 260343, 260344, 260345, 260346, 260347, 260348) c6(260349, 260350) c7(ae, ci, ad); #c1(HSD11B1L) c2(NP_941994) c3 (hydroxysteroid fl-beta-dehydrogenase 1-like protein isoform a precursor) c4(260351) c5(260352, 260353, 260354, 260355, 260356, 260357, 260358, 260359) c6(260360, 260361) c7(ae, ci, ad); #c1(HSD11B1L) c2(NP_941995) c3(hydroxysteroid 11-beta-dehydrogenase 1-like protein isoform b precursor) c4(260362, 260363, 260364) c5(260365, 260366, 260367, 260368, 260369, 260370, 260371, 260372, 260373, 260374, 260375, 260376, 260377, 260378, 260379, 260380, 260381, 260382, 260383, 260384, 260385, 260386, 260387, 260388, 260389) c6(260390, 260391, 260392, 260393, 260394, 260395) c7(ae, ci, ad); #c1(HSD11B1L) c2(NP_941996) c3(hydroxysteroid 11-beta-dehydrogenase 1-like protein isoform c) c4( )c5(260396, 260397, 260398, 260399, 260400, 260401, 260402) c6(260403, 260404) c7(ae, ci, ad); #c1(HSD11B2) c2(NP_000187) c3(corticosteroid 11-beta-dehydrogenase isozyme 2) c4(4134) c5(4135, 4136, 4137, 4138, 4139, 4140) c6( )c7(bM, jv, bLl, eR, DD, aZ, iW, e, al, aw, cz, fF, To, aj, v, ah, bB, fr, aY, q, J, ai, dP, AW, Tl, XD, bLJ, bLK, aT, vg, K, nJ, yo, A, s, aW, Fe, eH, r, aPz, iV, eF, t, uC, aiv, DL, qa, bLL, ck, hq, bE, afd, fP, tl, hR, dy, OD, ei, y, B, aOo); #c1(HSD17B1) c2(NP_000404) c3(estradiol 17-beta-dehydrogenase 1) c4(8564) c5(8565, 8566, 8567, 8568, 8569, 8570, 8571, 8572, 8573, 8574, 8575, 8576, 8577, 8578, 8579, 8580, 8581, 8582, 8583, 8584, 8585) c6( )c7(T, aZ, iW, aA, q, ad, dP, aT, dL, nJ, ae, qw, kl, PE, aW, ev, r, aF, bu, cl, zE, t, Ld, anZ, Pl, afd, hL, at, Lb, m); #c1(HSD17B1) c2(XP_011523032) c3(estradiol 17-beta-dehydrogenase I isoform X4) c4( )c5(317537, 317538) c6( )c7(T, aZ, iW, aA, q, ad, dP, aT, dL, nJ, ae, qw, kl, PE, aW, ev, r, aF, bu, cl, zE, t, Ld, anZ, Pl, afd, hl, at, Lb, m); #c1(HSD17B1) c2(XP_011523033) c3(estradiol 17-beta-dehydrogenase I isoform X7) c4(317539, 317540) c5(317541) c6( )c7(T, aZ, iW, aA, q, ad, dP, aT, dL, nJ, ae, qw, kl, PE, aW, ev, r, aF, bu, cl, zE, t, Ld, anZ, Pl, afd, hL, at, Lb, m); #c1(HSD(7B10) c2(NP_001032900) c3(3-hydroxyacyl-CoA dehydrogenase type-2 isoform 2) c4(31491) c5(31492, 31493, 31494, 31495, 31496, 31497, 31498, 31499, 31500, 31501, 31502) c6( )c7(bL, iu, aqg, eF, bLM, U, nf, jy, K, nA, f, H, bLN, af, aR, xt, L); #c1(HSD(7810) c2(NP_004484) c3(3-hydroxyacyl-CoA dehydrogenase type-2 isoform 1) c4(146809, 146810) c5(146811, 146812, 146813, 146814, 146815, 146816, 146817, 146818, 146819) c6( )c7(bL, iu, aqg, eF, bLM, U, nf, jy, K, nA, f, H, bLN, af, aR, xt, L); #c1(HSD17B11) c2(NP_057329) c3(estradiol 17-beta-dehydrogenase 11 precursor) c4(190149) c5(190150, 190151, 190152, 190153, 190154, 190155) c6( )c7(ae, ad); #c1(HSD(7B12) c2(NP_057226) c3(estradiol 17-beta-dehydrogenase 12) c4(189581) c5(189582, 189583, 189584, 189585, 189586, 189587, 189588, 189589) c6(189590, 189591) c7(ke, T, cF, hb, aW, qt, aw, aT, cm, ci, m, t, iK); #c1 (HSD17B13) c2(NP_001129702) c3(17-beta-hydroxysteroid dehydrogenase 13 isoform B) c4(52215) c5(52216, 52217, 52218, 52219, 52220, 52221) c6( )c7(ck, dP, T, qw, vJ, aW, PE, aZ, qt, afd, aT, aez, UD, Pp, v, m, nJ, ae, ad); #c1(HSD(7B13) c2(NP_835236) c3(17-beta-hydroxysteroid dehydrogenase 13 isoform A precursor) c4(253414) c5(253415, 253416, 253417, 253418, 253419, 253420) c6( )c7(ck, dP, T, qw, vJ, aW, PE, aZ, qt, afd, aT, aez, UD, Pp, v, m, nJ, ae, ad); #c1(HSD(7814) c2(NP_057330) c3(17-beta-hydroxysteroid dehydrogenase 14) c4(190156) c5(190157, 190158, 190159, 190160, 190161, 190162) c6( )c7(aw); #c1(HSD17B2) c2(NP_002144) c3(estradiol 17-beta-dehydrogenase 2) c4(125816) c5(125817, 125818, 125819, 125820, (25821, 125822, 125823) c6( )c7(aZ, iW, aA, bB, ml, q, ad, dP, aT, dL, ae, qw, kl, aW, r, fQ, aF, mo, cL, t, op, bN, bE, vA, afd, hL, at, m); #c1(HSD1783) c2(NP_000188) c3(testosterone (7-beta-dehydrogenase 3) c4(4141, 4142) c5(4143, 4144, 4145, 4146, 4147, 4148, 4149, 4150, 4151, 4152, 4153, 4154) c6( )c7(bL, aeQ, cF, UL, UB, Gs, r, adW, aez, aT, Pp, ae, ad, bN); #c1(HSD1784) c2(NP_000405) c3(peroxisomal multifunctional enzyme type 2 isoform 2) c4(8586) c5(8587, 8588, 8589, 8590, 8591, 8592) c6( )c7(bL, am, Ut, oe, jh, cz, aj, dG, v, V, qW, ai, ad, J, aQe, eA, eW, xy, il, aT, yB, HD, afV, dL, mL, K, cb, dF, ae, eL, bA, cC, A, mR, bu, Pd, zZ, t, cl, Ld, bN, hq, bLO, hL, mE, at, oh, bLP, Lb, pK); #c1(HSD1784) c2(NP_001186220) c3(peroxisomal multifunctional enzyme type 2 isoform 1) c4(77428, 77429) c5(77430, 77431, 77432, 77433, 77434, 77435, 77436) c6( )c7(bL, am, Ut, oe, jh, cz, aj, dG, v, V, qW, ai, ad, J, aQe, eA, eW, xy, il, aT, yB, hD, afV, dL, mL, K, cb, dF, ae, eL, bA, cC, A, mR, bu, Pd, zZ, t, cl, Ld, bN, hq, bLO, hL, mE, at, oh, bLP, Lb, pK); #c1(HSD(784) c2(NP_001186221) c3(peroxisomal multifunctional enzyme type 2 isoform 3) c4(77437) c5(77438, 77439, 77440, 77441, 77442, 77443) c6( )c7(bL, am, Ut, oe, jh, cz, aj, dG, v, V, qW, ai, ad, J, aQe, eA, eW, xy, il, aT, yB, hD, afV, dL, mL, K, cb, dF, ae, eL, bA, cC, A, mR, bu, Pd, zZ, t, cl, Ld, bN, hq, bLO, hL, mE, at, oh, bLP, Lb, pK); #c1(HSD1784) c2(NP_001278956) c3(peroxisomal multifunctional enzyme type 2 isoform 4) c4(111099, 111100) c5(111101, 111102, 111103, 111104, 111105, 111106, 111107, 111108, 111109, 111110, 111111) c6( ) c7(bL, am, Ut, oe, jh, cz, aj, dG, v, V, qW, ai, ad, J, aQe, eA, eW, xy, il, aT, yB, HD, afV, dL, mL, K, cb, dF, ae, eL, bA, cC, A, mR, bu, Pd, zZ, t, cl, Ld, bN, hq, bLO, hL, mE, at, oh, bLP, Lb, pK); #c1 (HSD1784) c2(NP_001278957) c3(peroxisomal multifunctional enzyme type 2 isoform 5) c4( )c5(111112, 111113, 111114, 111115, 111116) c6( )c7(bL, am, Ut, oe, jh, cz, aj, dG, v, V, qW, ai, ad, J, aQe, eA, eW, xy, il, aT, yB, hD, afV, dL, mL, K, cb, dF, ae, eL, bA, cC, A, mR, bu, Pd, zZ, t, cl, Ld, bN, hq, bLO, hL, mE, at, oh, bLP, Lb, pK); #c1(HSD17B6) c2(XP_006719735) c3(17-beta-hydroxysteroid dehydrogenase type 6 isoform X1) c4(292915, 292916, 292917) c5(292918, 292919, 292920, 292921, 292922, 292923, 292924, 292925, 292926, 292927, 292928, 292929, 292930, 292931, 292932, 292933, 292934, 292935, 292936, 292937, 292938, 292939, 292940, 292941, 292942, 292943, 292944, 292945, 292946, 292947, 292948, 292949, 292950, 292951) c6( )c7(bM, T, eR, WA, aZ, nx, ol, aA, tc, bZ, aj, Vd, PJ, J, ad, qC, fU, HF, av, yQ, hb, oj, aT, adX, Ak, bK, K, Gz, ae, ajU, ak, kl, aW, aM, az, hl, ff, fQ, So, dq, aF, HL, en, cl, Z, f, px, bz, eE, dr, bN, mO, adc, adZ, SE, dB, bE, awN, bo, U, hL, avF, eQ, anx, lM, ao); #c1(HSD1787) c2(NP_001291441) c3(3-keto-steroid reductase isoform 2) c4(116049) c5( )c6( )c7(T, fR, lT, aZ, cz, bB, hz, aj, v, ci, AO, q, J, ad, Hr, dP, vJ, aT, PD, UD, mL, uV, nJ, ae, af, qw, yo, kl, s, qt, PE, aW, r, Pp, cl, t, op, ck, bE, JA, vA, afd, hL, aez, m); #c1(HSD1787) c2(NP_001291442) c3(3-keto-steroid reductase isoform 3) c4( )c5(116050, 116051, 116052, 116053, 116054, 116055, 116056, 116057, 116058, 116059, 116060, 116061) c6( )c7(T, fR, lT, aZ, cz, bB, hz, aj, v, ci, AO, q, J, ad, Hr, dP, vJ, aT, PD, UD, mL, uV, nJ, ae, af, qw, yo, kl, s, qt, PE, aW, r, Pp, cl, t, op, ck, bE, JQ, vA, afd, hL, aez, m); #c1(HSD1787) c2(NP_057455) c3(3-keto-steroid reductase isoform 1) c4(190975, 190976, 190977, 190978) c5(190979, 190980, 190981, 190982, 190983, 190984, 190985, 190986, 190987, 190988, 190989, 190990, 190991, 190992, 190993, 190994, 190995, 190996) c6( ) c7(T, fR, lT, aZ, cz, bB, hz, aj, v, ci, AO, q, J, ad, Hr, dP, vJ, aT, PD, UD, mL, uV, nJ, ae, af, qw, yo, kl, s, qt, PE, aW, r, Pp, cl, t, op, ck, bE, JQ, vA, afd, hL, aez, m); #c1(HSD1788) c2(NP_055049) c3(estradiol 17-beta-dehydrogenase 8) c4(178580) c5(178581, 178582, 178583, 178584, 178585, 178586, 178587, 178588, 178589, 178590, 178591, 178592, 178593, 178594, 178595, 178596, 178597, 178598, 178599, 178600, 178601) c6( )c7(eZ, aT); #c1(HSD3B1) c2(XP_011539616) c3(3 beta-hydroxysteroid dehydrogenase/Delta 5->4-isomerase type I isoform X1) c4(337861, 337862, 337863) c5(337864, 337865, 337866, 337867, 337868, 337869, 337870, 337871, 337872) c6( ) c7(bL, G, iW, oe, cz, wo, ci, ad, dP, aT, dL, aUm, nJ, ae, eL, kl, zQ, PE, Pp, cl, qa, bJ, hl, at, aez, bko, aTW); #c1(HSD3B7) c2(NP_001136250) c3(3 beta-hydroxysteroid dehydrogenase type 7 isoform b) c4(54689) c5(54690, 54691, 54692, 54693, 54694, 54695, 54696, 54697, 54698, 54699) c6( )c7(afj, bLQ); #c1(HSD387) c2(NP_079469) c3(3 beta-hydroxysteroid dehydrogenase type 7 isoform a) c4(218209) c5(218210, 218211, 218212, 218213, 218214, 218215) c6( )c7(afj, bLQ); #c1(HSDL1) c2(NP_001139523) c3(inactive hydroxysteroid dehydrogenase-like protein I isoform b) c4(59464) c5(59465, 59466, 59467, 59468, 59469, 59470) c6( )c7(beX); #c1(HSDL1) c2(NP_113651) c3(inactive hydroxysteroid dehydrogenase-like protein I isoform a) c4(221949) c5(221950, 221951, 221952, 221953, 221954, 221955) c6( )c7(beX); #c1(HSDL2) c2(NP_001182751) c3(hydroxysteroid dehydrogenase-like protein 2 isoform 2) c4(75231) c5(75232, 75233, 75234, 75235, 75236, 75237, 75238) c6( )c7(Na, aq, Nc); #c1(HSDL2) c2(NP_115679) c3(hydroxysteroid dehydrogenase-like protein 2 isoform 1) c4(223519, 223520, 223521) c5(223522, 223523, 223524, 223525, 223526, 223527, 223528, 223529, 223530, 223531, 223532, 223533) c6( )c7(Na, aq, Nc); #c1(HSDL2) c2(XP_006717366) c3(hydroxysteroid dehydrogenase-like protein 2 isoform X2) c4( )c5(291011, 291012, 291013, 291014, 291015) c6( )c7(Na, aq, Nc); #c1(HSF1) c2(NP_005517) c3 (heat shock factor protein 1) c4(157016) c5(157017, 157018, 157019, 157020, 157021, 157022, 157023) c6( )c7(T, e, jh, aA, lb, n, aj, Fb, ci, C, PJ, qN, eA, J, ad, Kx, jz, aE, aT, DO, yT, hP, K, ae, B, aW, Y, ff, aq, en, cl, t, bz, es, ar, Ew, bP, c, eM, iA, gX, U, hL, m, R, bt, X); #c1(HSF2) c2(NP_001129036) c3(heat shock factor protein 2 isoform b) c4(50684) c5(50685, 50686, 50687, 50688, 50689, 50690) c6( )c7(vH, akv, K, iV); #c1(HSF2) c2(NP_004497) c3(heat shock factor protein 2 isoform a) c4(146936) c5(146937, 146938, 146939, 146940, 146941, 146942, 146943) c6( )c7(vH, akv, K, iV); #c1(HSF4) c2(NP_001035757) c3 (heat shock factor protein 4 isoform b) c4(33038) c5(33039, 33040, 33041, 33042, 33043, 33044, 33045, 33046, 33047, 33048) c6( )c7(F, bLR, aWG, jx, bLS, bwE, h, bLT, nA, lk, hX); #c1(HSF4) c2(NP_001529) c3(heat shock factor protein 4 isoform a) c4(118625, 118626) c5(118627, 118628, 118629, 118630, 118631, 118632, 118633, 118634, 118635, 118636, 118637, 118638, 118639) c6( )c7(F, bLR, aWG, jx, bLS, bwE, h, bLT, nA, lk, hX); #c1(HSF5) c2(NP_001073908) c3(heat shock factor protein 5) c4(36477, 36478) c5(36479, 36480, 36481, 36482, 36483, 36484, 36485, 36486, 36487, 36488, 36489, 36490) c6( )c7(Y); #c1(HSFY2) c2(NP_001001877) c3(heat shock transcription factor, Y-linked isoform 2) c4(19048) c5(19049, 19050, 19051, 19052, 19053, 19054, 19055, 19056, 19057, 19058, 19059, 19060, 19061, 19062, 19063, 19064, 19065, 19066, 19067) c6( ) c7(vH, pt, gU, Ul); #c1(HSFY2) c2(NP_714927) c3(heat shock transcription factor, Y-linked isoform 1) c4(246638, 246639) c5(246640, 246641, 246642, 246643, 246644, 246645, 246646, 246647, 246648, 246649, 246650) c6( )c7(vH, pt, gU, Ul); #c1(HSH2D) c2(NP_001278203) c3(hematopoietic SH2 domain-containing protein isoform 2) c4( )c5(109489) c6( )c7(TU, TR, ju, jh); #c1(HSH2D) c2(NP_116244) c3(hematopoietic SH2 domain-containing protein isoform 1) c4(225933) c5(225934, 225935, 225936, 225937, 225938, 225939, 225940) c6( )c7(TU, TR, ju, jh); #c1(HSH2D) c2(XP_011526691) c3(hematopoietic SH2 domain-containing protein isoform X3) c4(320989) c5(320990, 320991, 320992, 320993, 320994, 320995) c6( )c7(TU, TR, ju, jh); #c1(HSP90AA1) c2(NP_001017963) c3(heat shock protein HSP 90-alpha isoform 1) c4(26662) c5(26663, 26664, 26665, 26666, 26667, 26668, 26669, 26670, 26671, 26672, 26673, 26674, 26675, 26676) c6( )c7(gh, SJ, os, T, bM, aC, ea, if, bO, fd, bB, aTy, ci, bv, azY, aE, bW, dV, DO, K, hu, eL, bc, azu, aW, az, g, pL, eF, cm, iP, eM, nB, Kv, iA, iT, mx, U, cj, m, alm, iu, dD, ht, n, awr, ct, qN, bLU, ai, eW, Hx, ft, kH, h, dL, ox, A, bLX, PX, OX, fe, aq, yq, dw, adz, kT, bP, dB, Ei, fL, B, bLW, aA, cR, q, er, eA, Kx, hW, aT, cy, bLY, bq, ae, af, kl, iU, Y, r, en, cl, t, ck, nM, iM, gX, wR, np, yH, aws, bt, ao, Kd, nZ, kh, qb, dM, uG, jh, oZ, bS, dG, fu, eq, Nl, hF, mC, ad, av, hd, MS, Db, ji, lQ, fb, bLV, Dl, xX, d, je, ff, eH, cr, eZ, kD, aF, EZ, Fq, iV, Z, iR, bz, dr, es, bE, aS, Kj, XO, MC, X); #c1 (HSP90AA1) c2(NP_005339) c3(heat shock protein HSP 90-alpha isoform 2) c4(155232, 155233) c5(155234, 155235, 155236, 155237, 155238, 155239) c6( )c7(gh, SJ, os, T, bM, aC, ea, if, bO, fd, bB, aTy, ci, bv, azY, aE, bW, dV, DO, K, hu, eL, bc, azu, aW, az, g, pL, eF, cm, iP, eM, nB, Kv, iA, iT, mx, U, cj, m, alm, iu, dD, ht, n, awr, ct, qN, bLU, ai, eW, Hx, ft, kH, h, dL, ox, A, bLX, PX, OX, fe, aq, yq, dw, adz, kT, bP, dB, Ei, fL, B, bLW, aA, cR, q, er, eA, Kx, hW, aT, cy, bLY, bq, ae, af, kl, iU, Y, r, en, cl, t, ck, nM, iM, gX, wR, np, yH, aws, bt, ao, Kd, nZ, kh, qb, dM, uG, jh, oZ, bS, dG, fu, eq, Nl, hF, mC, ad, av, hd, MS, Db, ji, lQ, fb, bLV, Dl, xX, d, je, ff, eH, cr, eZ, kD, aF, EZ, Fq, iV, Z, iR, bz, dr, es, bE, aS, Kj, XO, MC, X); #c1(HSP90AB1) c2(NP_001258898) c3(heat shock protein HSP 90-beta isoform a) c4(96708, 96709, 96710, 96711, 96712, 96713, 96714) c5(96715, 96716, 96717, 96718, 96719, 96720, 96721, 96722, 96723, 96724, 96725, 96726, 96727, 96728, 96729) c6( )c7(bM, T, sw, fd, n, bS, ct, hF, ad, aE, Db, aT, h, bl, K, ox, fb, gj, aW, eZ, Z, bz, ck, ar, nM, bt, B); #c1(HSP90AB1) c2(NP_001258900) c3(heat shock protein HSP 90-beta isoform b) c4(96730) c5(96731, 96732, 96733, 96734, 96735, 96736, 96737, 96738, 96739, 96740, 96741, 96742, 96743, 96744, 96745, 96746, 96747, 96748, 96749, 96750, 96751, 96752, 96753, 96754, 96755, 96756, 96757, 96758, 96759, 96760, 96761, 96762, 96763, 96764, 96765, 96766, 96767, 96768, 96769) c6( )c7(bM, T, sw, fd, n, bS, ct, hF, ad, aE, Db, aT, h, bl, K, ox, fb, gj, aW, eZ, Z, bz, ck, ar, nM, bt, B); #c1(HSP90AB1) c2(NP_001258901) c3 (heat shock protein HSP 90-beta isoform c) c4(96770) c5(96771, 96772, 96773) c6( )c7(bM, T, sw, fd, n, bS, ct, hF, ad, aE, Db, aT, h, bl, K, ox, fb, gj, aW, eZ, Z, bz, ck, ar, nM, bt, B); #c1(HSP90B1) c2(NP_003290) c3(endoplasmin precursor) c4(137/79, 137180, 137181) c5(137182, 137183, 137184, 137185, 137186, 137187, 137188, 137189, 137190, 137191, 137192, 137193, 137194, 137195, 137196, 137197, 137198, 137199) c6( )c7(T, fR, mr, pf, if, jh, aA, fd, n, ame, ci, qN, q, ad, ai, brq, hX, eA, Bl, jz, aE, ju, gd, aT, cy, rO, bK, K, ak, azU, aGP, aW, Y, ff, eH, eZ, LZ, fe, kp, aF, en, g, dw, eF, cm, bz, iV, bLZ, aGo, hc, eD, Zn, y, B); #c1(HSPA12A) c2(NP_079291) c3(heat shock 70 kDa protein (2A) c4(217702) c5(217703, 217704, 217705, 217706, 217707, 217708) c6( )c7(lB, b0); #c1(HSPA12B) c2(NP_443202) c3(heat shock 70 kDa protein (28 isoform 1) c4(229872, 229873, 229874) c5(229875, 229876, 229877, 229878, 229879, 229880, 229881, 229882, 229883, 229884, 229885, 229886, 229887, 229888, 229889, 229890, 229891) c6( )c7(bM, e, C); #c1(HSPA12B) c2(XP_011527451) c3(heat shock 70 kDa protein 12B isoform X1) c4( )c5(322229) c6( )c7(bM, e, C); #c1(HSPA13) c2(NP_008879) c3(heat shock 70 kDa protein 13 precursor) c4(169462, 169463) c5(169464, 169465, 169466, 169467, 169468, 169469, 169470, 169471, 169472, 169473, 169474, 169475) c6( )c7(v, aA); #c1(HSPA14) c2(NP_001265134) c3(heat shock 70 kDa protein 14 isoform 3) c4(97820) c5(97821, 97822, 97823, 97824, 97825, 97826, 97827, 97828, 97829) c6( )c7(T, dD, NQ, aBe, fR, sQ, ea, aD, yG, aA, fd, cL, bZ, j, ci, aj, v, eq, ai, o, J, ad, xE, aEE, aV, qG, azm, K, lQ, ae, avn, eL, Dl, A, mR, az, Pg, aOW, lo, aF, EZ, g, as, kk, t, f, cm, iV, y, bMb, AH, aDj, U, uo, uz, bMa, ut, at, m, fD, lf, aEw); #c1(HSPA14) c2(NP_057383) c3(heat shock 70 kDa protein 14 isoform 1) c4(190484, 190485) c5(190486, 190487, 190488, 190489, 190490, 190491, 190492, 190493, 190494, 190495, 190496, 190497) c6( )c7(T, dD, NQ, aBe, fR, sQ, ea, aD, yG, aA, fd, cL, bZ, j, ci, aj, v, eq, ai, o, J, ad, xE, aEE, aV, qG, azm, K, lQ, ae, avn, eL, Dl, A, mR, az, Pg, aOW, lo, aF, EZ, g, as, kk, t, f, cm, iV, y, bMb, AH, aDj, U, uo, uz, bMa, ut, at, m, fD, lf, aEw); #c1(HSPA1A) c2(NP_005336) c3(heat shock 70 kDa protein (1A/1B) c4(155057, 155058, 155059, 155060, 155061, 155062, 155063, 155064, 155065) c5(155066, 155067, 155068, 155069, 155070, 155071, 155072, 155073, 155074, 155075, 155076, 155077, 155078, 155079, 155080, 155081, 155082, 155083, 155084, 155085, 155086, 155087, 155088, 155089, 155090, 155091, 155092, 155093, 155094, 155095, 155096, 155097, 155098, 155099, 155100, 155101, 155102, 155103, 155104, 155105, 155106, 155107, 155108, 155109, 155110, 155111, 155112, 155113, 155114, 155115, 155116, 155117, 155118, 155119, 155120, 155121, 155122, 155123, 155124, 155125, 155126, 155127, 155128, 155129, 155130, 155131, 155132, 155133, 155134, 155135, 155136, 155137, 155138, 155139, 155140, 155141, 155142, 155143, 155144, 155145, 155146, 155147, 155148, 155149, 155150, 155151, 155152, 155153, 155154, 155155, 155156, 155157, 155158, 155159, 155160, 155161, 155162, 155163, 155164, 155165, 155166, 155167, 155168, 155169, 155170, 155171, 155172, 155173, 155174, 155175, 155176, 155177, 155178, 155179, 155180, 155181, 155182, 155183, 155184, 155185, 155186, 155187, 155188, 155189, 155190, 155191, 155192, 155193, 155194, 155195, 155196, 155197, 155198, 155199, 155200, 155201, 155202, 155203, 155204, 155205, 155206, 155207, 155208, 155209, 155210, 155211, 155212, 155213, 155214, 155215, 155216, 155217) c6( )c7(bM, T, fR, ea, e, bO, aza, fd, qj, ci, aY, fS, M, dV, K, eL, eG, aW, az, hl, vs, cm, uC, eM, Fh, acS, U, uo, tG, dD, gU, sQ, tp, aWG, qy, aj, fr, qW, cd, Hx, ft, h, dL, vd, wb, A, mR, fe, CQ, hq, bMc, kT, ar, vL, E, ut, eo, gg, sZ, B, bL, aA, fF, tc, bJw, j, v, C, V, eA, J, mP, qp, ld, aT, cy, an, ae, af, kl, Y, wA, ck, ac, pZ, hA, fP, CZ, fD, vi, jh, vr, eq, iG, qi, sB, aGW, ad, hD, lQ, yF, uQ, d, aYu, eZ, dq, aF, hp, iV, bz, Hv, qa, eK, uR, aS, y, X, BA, pC); #c1(HSPA1L) c2(NP_005518) c3(heat shock 70 kDa protein 1-like) c4(157024, 157025, 157026) c5(157027, 157028, 157029, 157030, 157031, 157032, 157033, 157034, 157035, 157036, 157037, 157038, 157039, 157040, 157041, 157042, 157043, 157044, 157045, 157046, 157047, 157048, 157049, 157050, 157051, 157052, 157053, 157054, 157055, 157056, 157057, 157058, 157059, 157060, 157061, 157062, 157063, 157064, 157065, 157066, 157067, 157068) c6( )c7(bL, T, DM, fR, sQ, tp, wL, bO, aA, cz, jh, j, jE, qi, qW, ad, ga, fS, ld, aT, h, K, lQ, an, ae, bA, uQ, A, mR, aW, eZ, uC, bz, qa, ck, bMc, kT, ac, pZ, dO, uR, vL, uo, ut, bMd, gT, sa, sZ, B, y, vi); #c1 (HSPA1L) c2(XP_005249127) c3(heat shock 70 kDa protein 1-like isoform X2) c4(269044) c5(269045, 269046, 269047, 269048) c6( )c7(bL, T, DM, fR, sQ, tp, wL, bO, aA, cz, jh, j, jE, qi, qW, ad, ga, fS, ld, aT, h, K, lQ, an, ae, bA, uQ, A, mR, aW, eZ, uC, bz, qa, ck, bMc, kT, ac, pZ, dO, uR, vL, uo, ut, bMd, gT, sa, sZ, B, y, vi); #c1(HSPA1L) c2(XP_005272874) c3(heat shock 70 kDa protein 1-like isoform X1) c4(284205, 284206) c5( )c6( )c7(bL, T, DM, fR, sQ, tp, wL, bO, aA, cz, jh, j, jE, qi, qW, ad, ga, fS, ld, aT, h, K, lQ, an, ae, bA, uQ, A, mR, aW, eZ, uC, bz, qa, ck, bMc, kT, ac, pZ, dO, uR, vL, uo, ut, bMd, gT, sa, sZ, B, y, vi); #c1(HSPA1L) c2(XP_005274916) c3(heat shock 70 kDa protein 1-like isoform X2) c4( )c5(285530, 285531, 285532, 285533, 285534, 285535, 285536, 285537, 285538, 285539) c6( )c7(bL, T, DM, fR, sQ, tp, wL, bO, aA, cz, jh, j, jE, qi, qW, ad, ga, fS, ld, aT, h, K, lQ, an, ae, bA, uQ, A, mR, aW, eZ, uC, bz, qa, ck, bMc, kT, ac, pZ, dO, uR, vL, uo, ut, bMd, gT, sa, sZ, B, y, vi); #c1(HSPA1L) c2(XP_005274919) c3(heat shock 70 kDa protein 1-like isoform X1) c4(285540) c5( )c6( )c7(bL, T, DM, fR, sQ, tp, wL, bO, aA, cz, jh, j, jE, qi, qW, ad, ga, fS, ld, aT, h, K, lQ, an, ae, bA, uQ, A, mR, aW, eZ, uC, bz, qa, ck, bMc, kT, ac, pZ, dO, uR, vL, uo, ut, bMd, gT, sa, sZ, B, y, vi); #c1 (HSPA1L) c2(XP_011545549) c3(heat shock 70 kDa protein 1-like isoform X2) c4( )c5(344277, 344278, 344279, 344280, 344281, 344282, 344283, 344284, 344285, 344286, 344287, 344288, 344289, 344290, 344291, 344292, 344293, 344294, 344295, 344296, 344297, 344298, 344299, 344300, 344301, 344302, 344303, 344304) c6( )c7(bL, T, DM, fR, sQ, tp, wL, bO, aA, cz, jh, j, jE, qi, qW, ad, ga, fS, ld, aT, h, K, lQ, an, ae, bA, uQ, A, mR, aW, eZ, uC, bz, qa, ck, bMc, kT, ac, pZ, dO, uR, vL, uo, ut, bMd, gT, sa, sZ, B, y, vi); #c1(HSPA2) c2(NP_068814) c3(heat shock-related 70 kDa protein 2) c4(210792, 210793) c5(210794, 210795, 210796, 210797, 210798, 210799, 210800, 210801, 210802, 210803, 210804, 210805, 210806, 210807, 210808, 210809, 210810) c6( )c7(bL, T, dD, gU, ea, jh, aA, lD, vr, aj, v, j, eq, C, fr, iG, cd, J, ad, qp, ju, ld, aT, hD, wb, K, ae, af, A, mR, aW, eZ, kt, fe, vs, aF, iV, bz, ar, fT, ac, eM, hA, fL, X); #c1(HSPA4) c2(NP_002145) c3(heat shock 70 kDa protein 4) c4(125824, 125825, 125826) c5(125827, 125828, 125829, 125830, 125831, 125832, 125833, 125834, 125835, 125836, 125837, 125838, 125839, 125840, 125841, 125842, 125843, 125844) c6( )c7(bM, T, fR, ea, if, bO, fd, bB, qj, ci, lR, aY, x, xE, fm, bW, pX, M, dV, K, hH, brQ, eL, hm, aiV, aW, aEl, az, eF, cm, bw, eM, Kv, aDs, acS, wE, mx, U, cj, uo, tG, k, m, pw, xh, cE, dt, dD, sQ, aWG, aw, aj, fr, Qh, qN, PJ, cd, ai, eW, Mo, Hx, h, lG, yT, aPe, dL, ey, bl, eV, pl, xf, A, fe, aq, CQ, beP, adz, bP, dB, at, ut, Lq, rx, eo, amb, sZ, B, ek, bL, jv, lk, dA, mr, AL, fG, eX, aA, cz, tc, fF, j, C, V, q, J, er, eA, ady, Kx, qp, bza, ld, aT, cy, bK, aDt, an, ae, af, ak, s, iU, Y, Pg, r, lo, en, wA, t, pb, dS, ck, rR, kP, c, ac, hA, wO, mG, hr, fP, aan, bt, aDj, LS, ao, wi, jh, eq, hF, ad, dP, ib, ds, hD, lQ, nJ, uQ, Dl, CL, d, ff, eH, eZ, aF, EZ, iV, Bu, bz, dr, qa, ek, LB, D, pc, bE, dO, wz, aS, aGg, Kj, y, tP, iu, pC); #c1(HSPA4L) c2(NP_055093) c3(heat shock 70 kDa protein 4L) c4(178887) c5( )c6( )c7(ck, K, bA); #c1(HSPA5) c2(NP_005338) c3(78 kDa glucose-regulated protein precursor) c4(155218, 155219) c5(155220, 155221, 155222, 155223, 155224, 155225, 155226, 155227, 155228, 155229, 155230, 155231) c6( )c7(T, aC, al, fd, bB, ame, ci, x, aE, K, oi, cp, li, eG, adr, Jl, aW, fc, g, as, eF, cm, eM, Jr, nB, wE, Jp, U, fy, uL, xN, bMe, n, aj, fr, eW, ft, jz, bKs, lG, dL, bl, alM, A, mR, lb, aq, hL, it, B, bL, jv, fM, aA, v, j, q, J, eA, HC, aT, cy, bK, an, ae, af, ak, hM, s, iU, Y, r, aMA, en, cL, t, ik, ck, kB, aGi, id, jh, dG, ad, hX, av, akN, hd, oj, ahK, nJ, d, qt, ff, eH, aF, iR, bz, gk, bE, cP, ei, X, nQ); #c1(HSPA6) c2(NP_002146) c3(heat shock 70 kDa protein 6) c4(125845, 125846) c5(125847, 125848, 125849, 125850, 125851, 125852, 125853, 125854, 125855, 125856, 125857, 125858) c6( )c7(T, fR, sQ, cj, uo, ut, aS, lQ, K, ae, ad); #c1(HSPA8) c2(NP_006588) c3(heat shock cognate 71 kDa protein isoform 1) c4(166425, 166426, 166427, 166428) c5(166429, 166430, 166431, 166432, 166433, 166434, 166435, 166436, 166437, 166438, 166439, 166440, 166441, 166442, 166443, 166444, 166445, 166446, 166447, 166448, 166449, 166450, 166451, 166452, 166453, 166454, 166455, 166456, 166457, 166458, 166459, 166460, 166461, 166462, 166463, 166464, 166465, 166466, 166467, 166468, 166469, 166470, 166471, 166472) c6(166473, 166474) c7(bM, T, BD, fR, eR, wL, aA, cz, bB, fd, j, ci, aY, eA, ad, eW, hX, Hx, akN, hw, ds, aT, aU, aNp, K, an, ae, af, eu, iU, aW, Y, ff, xs, dq, en, g, bz, kB, bE, E, be, ei, y, bBl, X, ao); #c1(HSPA8) c2(NP_694881) c3(heat shock cognate 71 kDa protein isoform 2) c4(245411) c5(245412, 245413, 245414, 245415, 245416, 245417, 245418, 245419) c6( )c7(bM, T, BD, fR, eR, wL, aA, cz, bB, fd, j, ci, aY, eA, ad, eW, hX, Hx, akN, hw, ds, aT, aU, aNp, K, an, ae, af, eu, iU, aW, Y, ff, xs, dq, en, g, bz, kB, bE, E, be, ei, y, bBl, X, ao); #c1(HSPA9) c2(NP_004125) c3(stress-70 protein, mitochondrial precursor) c4(143776, 143777, 143778) c5(143779, 143780, 143781, 143782, 143783, 143784, 143785, 143786, 143787, 143788, 143789, 143790, 143791, 143792, 143793, 143794, 143795, 143796) c6( )c7(T, aA, bB, n, j, ci, fr, qW, q, aR, br, ad, ke, MR, aXH, jZ, dV, h, dz, KP, K, jo, an, ae, af, eu, gC, r, gl, aq, aF, La, iR, cm, bw, dr, ck, iT, bE, U, wz, bt, B); #c1(HSPB1) c2(NP_001531) c3(heat shock protein beta-1) c4(118651, 118652) c5(118653, 118654, 118655, 118656, 118657, 118658, 118659, 118660, 118661, 118662, 118663, 118664, 118665) c6( )c7(bM, T, fR, pf, ea, e, fd, bB, ci, aR, x, aE, ws, bMh, ja, K, bU, H, eL, eG, Jl, aW, az, hl, LZ, g, eF, cm, iP, eM, Kv, U, cj, k, m, xh, cE, iu, dD, gU, Ol, aw, qy, aj, fr, asF, PJ, cd, b, h, lG, bMf, dL, bV, bT, aq, aoO, RB, aWn, amb, cB, B, L, DD, aA, j, bX, bgR, V, awg, J, q, eA, aT, cy, bMg, ae, kl, vT, Y, r, en, bu, t, kC, iK, ck, alC, ac, c, aXe, wO, bko, fD, im, bt, bCG, LS, tS, eP, Zl, Pt, aZ, jh, eq, fu, ad, brR, av, py, vc, nJ, iL, d, ff, eH, aF, bz, aZz, ek, bE, i, y, X, bsJ); #c1(HSPB2) c2(NP_001532) c3(heat shock protein beta-2) c4(118666) c5(118667, 118668, 118669, 118670, 118671, 118672, 118673, 118674, 118675, 118676, 118677, 118678, 118679) c6( )c7(bM, T, pf, ea, e, fd, bB, ci, aR, aE, ws, ja, K, bU, H, eL, eG, Jl, aW, az, g, eF, cm, iP, Kv, U, cj, m, xh, cE, dD, aw, qy, aj, asF, Hx, b, bMf, dL, bV, cM, aoO, RB, aWn, amb, it, B, L, DD, aA, j, bgR, V, q, J, eA, aT, cy, ae, kl, vT, Y, r, en, bu, t, kC, iK, ck, ac, c, aXe, wO, bko, fD, bt, LS, tS, ob, Zl, aZ, jh, eq, fu, ad, brR, py, nJ, iL, d, ff, eH, aF, bz, aZz, ek, bE, i, y, bsJ); #c1(HSPB3) c2(NP_006299) c3(heat shock protein beta-3) c4(163810) c5(163811, 163812, 163813, 163814, 163815, 163816, 163817) c6( )c7(bM, T, pf, ea, e, al, fd, bB, Vd, ci, aR, aE, ws, dV, ja, K, qM, bU, hu, H, eL, eG, Jl, aW, az, g, eF, cm, iP, eM, Kv, U, cj, m, eg, hh, xh, cE, dD, aw, ht, qy, aj, asf, ai, b, aZv, bMf, dL, bV, mo, aoO, RB, aWn, hL, amb, B, qc, bL, L, DD, fG, jx, aA, v, j, bgR, q, J, eA, aT, kf, cy, rE, ae, ajU, kl, vT, Y, aM, r, lo, en, bu, cl, t, kC, iK, ck, ac, c, aXe, wO, AH, Uq, bko, fD, bt, LS, Mb, tS, Zl, aZ, jh, Hn, eq, fu, ad, brR, py, ds, oW, nJ, iL, d, ff, eH, eZ, aF, bz, aZz, ek, gk, bE, i, y, bMi, bsJ); #c1(HSPB6) c2(NP_653218) c3(heat shock protein beta-6) c4(239150, 239151) c5(239152, 239153, 239154, 239155, 239156, 239157, 239158, 239159, 239160, 239161, 239162, 239163, 239164, 239165, 239166, 239167, 239168, 239169, 239170) c6( )c7(fV, fm, mx, gZ, cy, akA, j, K, B, L); #c1(HSPB7) c2(XP_011539551) c3(heat shock protein beta-7 isoform X1) c4(337759, 337760, 337761) c5(337762, 337763, 337764, 337765, 337766, 337767, 337768, 337769, 337770, 337771, 337772, 337773) c6( )c7(T, wH, mx, uY, fr, mo, cd); #c1(HSPB7) c2(XP_011539552) c3(heat shock protein beta-7 isoform X2) c4( )c5(337774, 337775) c6( )c7(T, wH, mx, uY, fr, mo, cd); #c1(HSPB8) c2(NP_055180) c3(heat shock protein beta-8) c4(179612) c5(179613, 179614, 179615, 179616, 179617, 179618) c6( )c7(T, Ol, l, aA, jh, n, hy, fu, bX, aR, ad, aao, brR, bMh, aT, kf, Ak, bMf, dL, K, bU, bMk, bV, ae, af, d, BN, iU, aW, bT, Y, aF, hf, aKv, bMj, ck, ac, c, Kv, U, at, ano, R, y, ne, L); #c1(HSPB9) c2(NP_149971) c3(heat shock protein beta-9) c4(227667) c5(227668, 227669, 227670, 227671, 227672, 227673) c6( )c7(bu); #c1(HSPBAP1) c2(NP_078886) c3(HSPB1-associated protein 1) c4(215908) c5(215909, 215910, 215911, 215912, 215913, 215914) c6( )c7(ha, K); #c1(HSPBAP1) c2(XP_011511432) c3(HSPB1-associated protein I isoform X3) c4( )c5(302072) c6( )c7(ha, K); #c1(HSPBAP1) c2(XP_011511433) c3(HSPB1-associated protein I isoform X5) c4(302073) c5(302074, 302075, 302076, 302077, 302078) c6( )c7(ha, K); #c1(HSPBP1) c2(NP_001123578) c3(hsp70-binding protein 1 isoform 2) c4(47723) c5(47724, 47725, 47726, 47727, 47728, 47729, 47730, 47731, 47732, 47733, 47734, 47735, 47736) c6( )c7(dB, av, y); #c1(HSPBP1) c2(NP_001284529) c3(hsp70-binding protein I isoform 1) c4( )c5(111980, 111981, 111982) c6( )c7(dB, av, y); #c1(HSPBP1) c2(XP_005258758) c3(hsp70-binding protein I isoform X1) c4(275727) c5( )c6( )c7(dB, av, y); #c1(HSPD1) c2(NP_002147) c3(60 kDa heat shock protein, mitochondrial) c4(125859, 125860, 125861, 125862, 125863, 125864, 125865, 125866) c5(125867, 125868, 125869, 125870, 125871, 125872, 125873, 125874, 125875, 125876, 125877, 125878, 125879, 125880, 125881, 125882, 125883, 125884, 125885, 125886, 125887, 125888, 125889, 125890, 125891, 125892, 125893, 125894, 125895, 125896, 125897, 125898, 125899, 125900, 125901, 125902, 125903, 125904, 125905, 125906, 125907, 125908, 125909, 125910, 125911, 125912, 125913, 125914, 125915, 125916, 125917, 125918) c6( )c7(T, fR, aFg, ea, e, fd, cL, ci, AW, xE, dY, bMp, azm, K, hH, eL, bc, VF, aEV, az, aOW, aDn, g, as, cm, U, cj, m, abh, aEw, dt, dD, aBe, aw, aj, fr, ai, eW, jz, cc, eT, h, hB, A, mR, aq, nN, bMl, kk, cM, bMb, aOL, bm, at, uz, bMa, B, vQ, ul, NQ, wL, aee, aA, cz, bZ, v, j, C, o, J, aEE, rS, cy, qG, bq, ae, af, bMn, Ue, fO, bMm, Pg, aEz, lo, t, f, iK, aff, AH, yH, fD, ao, aDj, wi, bMo, aD, lD, dG, eq, aGW, ad, hX, abZ, aV, io, vc, jo, Dl, blU, aFr, aF, si, hp, EZ, iV, Bv, lf, y); #c1(HSPE1) c2(NP_002148) c3(10 kDa heat shock protein, mitochondrial) c4(125919, 125920, 125921) c5(125922, 125923, 125924, 125925, 125926, 125927, 125928, 125929, 125930, 125931, 125932, 125933, 125934, 125935, 125936, 125937, 125938, 125939, 125940) c6( )c7(T, Yn, AW, aEE, aV, cc, zw, hL, eZ, h, aq, bJC, K, m, ci, uE, t, B, abh); #c1(HSPG2) c2(NP_001278789) c3(basement membrane-specific heparan sulfate proteoglycan core protein isoform a precursor) c4(110086, 110087) c5( )c6( )c7(xS, T, aPl, arX, aao, aE, us, K, hu, dQ, akT, eG, apl, aW, Az, uY, zC, uC, bhB, nB, cj, m, bmE, alo, dD, ht, oY, ee, cd, h, he, A, bMq, aq, cv, iO, w, ln, bm, uz, B, bL, AS, bll, dA, cz, v, j, q, xx, qK, aT, jj, bq, rv, ae, af, Y, HL, en, ck, vW, Nk, dy, sY, aaT, kh, eq, XM, ad, LH, hX, HF, F, Db, uD, anN, TY, ff, aF, iR, bz, wW, bE, i, hE, EB); #c1(HSPG2) c2(XP_011539619) c3(basement membrane-specific heparan sulfate proteoglycan core protein isoform X1) c4( )c5(337873, 337874, 337875, 337876, 337877, 337878, 337879, 337880, 337881, 337882, 337883, 337884, 337885, 337886, 337887, 337888, 337889, 337890, 337891, 337892, 337893, 337894, 337895, 337896, 337897, 337898, 337899, 337900, 337901, 337902, 337903, 337904, 337905, 337906, 337907) c6( )c7(xS, T, aPl, arX, aao, aE, us, K, hu, dQ, akT, eG, apl, aW, Az, uY, zC, uC, bhB, nB, cj, m, bmE, aLo, dD, ht, oY, ee, cd, h, he, A, bMq, aq, cv, iO, w, ln, bm, uz, B, bL, AS, bll, dA, cz, v, j, q, xx, qK, aT, jj, bq, rv, ae, af, Y, HL, en, ck, vW, Nk, dy, sY, aaT, kh, eq, XM, ad, LH, hX, HF, F, Db, uD, anN, TY, ff, aF, iR, bz, wW, bE, i, HE, EB); #c1(HSPG2) c2(XP_011539621) c3(basement membrane-specific heparan sulfate proteoglycan core protein isoform X6) c4(337908) c5( )c6( ) c7(xS, T, aPl, arX, aao, aE, us, K, hu, dQ, akT, eG, apl, aW, Az, uY, zC, uC, bhB, nB, cj, m, bmE, aLo, dD, ht, oY, ee, cd, h, he, A, bMq, aq, cv, iO, w, ln, bm, uz, B, bL, AS, bll, dA, cz, v, j, q, xx, qK, aT, jj, bq, rv, ae, af, Y, HL, en, ck, vW, Nk, dy, sY, aaT, kh, eq, XM, ad, LH, hX, HF, F, Db, uD, anN, TY, ff, aF, iR, bz, wW, bE, i, HE, EB); #c1(HSPG2) c2(XP_011539623) c3(basement membrane-specific heparan sulfate proteoglycan core protein isoform X8) c4(337909) c5( )c6( )c7(xS, T, aPl, arX, aao, aE, us, K, hu, dQ, akT, eG, apl, aW, Az, uY, zC, uC, bhB, nB, cj, m, bmE, aLo, dD, ht, oY, ee, cd, h, he, A, bMq, aq, cv, iO, w, ln, bm, uz, B, bL, AS, bll, dA, cz, v, j, q, xx, qK, aT, jj, bq, rv, ae, af, Y, HL, en, ck, vW, Nk, dy, sY, aaT, kh, eq, XM, ad, LH, hX, HF, F, Db, uD, anN, TY, ff, aF, iR, bz, wW, bE, i, hE, EB); #c1(HSPG2) c2(XP_011539624) c3(basement membrane-specific heparan sulfate proteoglycan core protein isoform X10) c4( )c5(337910, 337911, 337912) c6( )c7(xS, T, aPl, arX, aao, aE, us, K, hu, dQ, akT, eG, apl, aW, Az, uY, zC, uC, bhB, nB, cj, m, bmE, aLo, dD, ht, oY, ee, cd, h, he, A, bMq, aq, cv, iO, w, ln, bm, uz, B, bL, AS, bll, dA, cz, v, j, q, xx, qK, aT, jj, bq, rv, ae, af, Y, HL, en, ck, vW, Nk, dy, sY, aaT, kh, eq, XM, ad, LH, hX, HF, F, Db, uD, anN, TY, ff, aF, iR, bz, wW, bE, i, HE, EB); #c1(HSPH1) c2(NP_001273432) c3(heat shock protein 105 kDa isoform 2) c4( )c5(104881) c6( )c7(T, Hx, fM, aE, oV, iW, jh, r, h, fr, K, q, fq, B); #c1(HSPH1) c2(NP_001273433) c3(heat shock protein 105 kDa isoform 3) c4(104882) c5(104883, 104884, 104885, 104886, 104887, 104888) c6( )c7(T, Hx, fM, aE, oV, iW, jh, r, h, fr, K, q, fq, B); #c1(HSPH1) c2(NP_001273434) c3(heat shock protein 105 kDa isoform 4) c4(104889) c5(104890, 104891, 104892, 104893, 104894, 104895) c6( )c7(T, Hx, fM, aE, oV, iW, jh, r, h, fr, K, q, fq, B); #c1(HSPH1) c2(NP_006635) c3(heat shock protein 105 kDa isoform 1) c4(166761, 166762) c5(166763, 166764, 166765, 166766, 166767, 166768, 166769, 166770, 166771, 166772, 166773, 166774, 166775) c6( )c7(T, Hx, fM, aE, oV, iW, jh, r, h, fr, K, q, fq, B); #c1(HSPH1) c2(XP_011533189) c3(heat shock protein 105 kDa isoform X1) c4(329323) c5(329324, 329325, 329326, 329327, 329328) c6( )c7(T, Hx, fM, aE, oV, iW, jh, r, h, fr, K, q, fq, B); #c1(HTAT1P2) c2(NP_001091990) c3(oxidoreductase HTAT1P2 isoform a precursor) c4(39164, 39165) c5(39166, 39167, 39168, 39169, 39170, 39171, 39172, 39173) c6( )c7(T, aA, n, ci, V, q, ad, av, iu, ft, aE, aT, h, K, ae, s, aW, Y, oV, ff, r, kp, en, g, bz, eM, B); #c1(HTAT1P2) c2(NP_001091991) c3(oxidoreductase HTAT1P2 isoform b) c4( )c5(39174, 39175, 39176, 39177, 39178, 39179, 39180, 39181, 39182, 39183, 39184, 39185) c6( )c7(T, aA, n, ci, V, q, ad, av, iu, ft, aE, aT, h, K, ae, s, aW, Y, oV, ff, r, kp, en, g, bz, eM, B); #c1(HTAT1P2) c2(NP_001091993) c3(oxidoreductase HTAT1P2 isoform c) c4(39186) c5(39187, 39188, 39189, 39190, 39191, 39192, 39193, 39194, 39195, 39196, 39197, 39198, 39199, 39200) c6( )c7(T, aA, n, ci, V, q, ad, av, iu, ft, aE, aT, h, K, ae, s, aW, Y, oV, ff, r, kp, en, g, bz, eM, B); #c1(HTN1) c2(NP_002150) c3(histatin-1 precursor) c4(125962, 125963) c5(125964, 125965, 125966, 125967, 125968, 125969, 125970, 125971, 125972, 125973) c6( )c7(ck, iT, acG, acH, it, bN); #c1(HTN3) c2(NP_000191) c3(histatin-3 precursor) c4(4255, 4256, 4257, 4258, 4259, 4260) c5(4261, 4262, 4263, 4264, 4265, 4266, 4267, 4268, 4269, 4270) c6( )c7(T, dD, lT, ea, aA, amj, eq, ci, eh, no, jZ, dV, aT, dL, bHF, yo, s, aW, Y, ck, iT, uz, R); #c1 (HTR1A) c2(NP_000515) c3(5-hydroxytryptamine receptor (A) c4(10875, 10876) c5(10877, 10878, 10879, 10880, 10881, 10882, 10883, 10884, 10885, 10886, 10887, 10888) c6( )c7(bMr, bcq, tJ, aoU, ht, bO, aA, bMs, bS, gf, ct, uu, ww, Ge, qi, il, wy, aR, xt, aqN, PW, F, ot, w, DK, QU, ta, abj, bK, K, an, he, af, na, aqv, A, tb, yL, eZ, akY, cu, EZ, g, bQ, Z, eE, cM, bN, cv, hq, pH, aqr, W, cA, HP, ZT, HK, Ei, bMt, VU, el, sv, Rm, aTU, uJ, vL, aqD); #c1(HTR1B) c2(NP_000854) c3(5-hydroxytryptamine receptor (B) c4(16589) c5(16590, 16591, 16592, 16593, 16594, 16595) c6( )c7(lD, uk, iH, jd, ago, bO, cz, n, To, bS, uu, ct, ci, mw, Ge, bMu, v, il, aQF, ad, cF, zw, ww, iX, aqk, cy, dL, bK, K, rE, he, rz, xa, na, ly, aog, tb, up, cu, cl, Z, akb, cm, bQ, FT, lx, bJ, hq, bN, ok, ayt, cA, ZT, Gs, bhU, hL, vL, at, Rm, uJ, Gp, lC, aXu, ra, qc); #c1 (HTR1E) c2(XP_011534092) c3(5-hydroxytryptamine receptor 1E isoform X1) c4(330323) c5(330324, 330325, 330326, 330327, 330328, 330329, 330330, 330331, 330332, 330333) c6( )c7(aw, bS, uu, wy); #c1(HTR1F) c2(NP_000857) c3(5-hydroxytryptamine receptor (F) c4(16596) c5(16597, 16598, 16599, 16600, 16601, 16602) c6( )c7(mU, HF, bBC, af, mW, mZ, mS, mY, bS, bK, uu, mV, mT, mX); #c1(HTR2A) c2(NP_000612) c3(5-hydroxytryptamine receptor 2A isoform 1) c4(13769) c5(13770, 13771, 13772, 13773, 13774, 13775, 13776, 13777, 13778, 13779) c6( )c7(bM, uK, aUk, e, al, bO, gf, Jd, ur, HH, VV, lu, iX, Fo, M, ta, HR, K, oi, H, hm, aW, bMC, Eg, BE, cm, uC, mZ, bhB, aCT, ZT, Gs, U, Ji, sv, uJ, cE, bok, aw, To, aj, ww, ct, cd, ke, aUb, mV, mY, Cr, he, pi, mU, aga, vo, cM, mX, cv, bJ, hq, ok, ar, bP, cA, aDu, yP, bMw, mg, bhU, VU, vL, at, jd, mT, L, bL, qc, iH, He, cz, mS, ti, bMB, j, v, Qo, il, wy, J, PW, bMz, CF, DK, aVM, sl, aql, aT, aaw, bK, bq, bMy, rz, an, rE, af, yL, bcD, cu, lo, HL, en, xw, pb, bN, sS, ac, azK, lw, gO, ao, sY, qX, akd, ha, mw, bS, uu, dG, abZ, HF, nH, mW, sO, aqk, vc, bMv, nJ, na, tb, cW, aal, Gv, ff, cr, kD, gl, dq, EZ, Z, jb, bMx, qa, gk, HP, bMA, gZ, Rm, ei, y, ra); #c1(HTR2A) c2(NP_001159419) c3(5-hydroxytryptamine receptor 2A isoform 2) c4(63990) c5(63991, 63992, 63993, 63994, 63995, 63996) c6( ) c7(bM, uk, aUk, e, al, bO, gf, Jd, ur, HH, VV, lu, iX, Fo, M, ta, HR, K, oi, H, hm, aW, bMC, Eg, eE, cm, uC, mZ, bhB, aCT, ZT, Gs, U, Ji, sv, uJ, cE, bok, aw, To, aj, ww, ct, cd, ke, aUb, mV, mY, Cr, he, pi, mU, aga, vo, cM, mX, cv, bJ, hq, ok, ar, bP, cA, aDu, yP, bMw, mg, bhU, VU, vL, at, jd, mT, L, bL, qc, iH, He, cz, mS, ti, bMB, j, v, Qo, il, wy, J, PW, bMz, cF, DK, aVM, sl, aql, aT, aaw, bK, bq, bMy, rz, an, rE, af, yL, bcD, cu, lo, HL, en, xw, pb, bN, sS, ac, azK, lw, gO, ao, sY, qX, akd, ha, mw, bS, uu, dG, abZ, HF, nH, mW, sO, aqk, vc, bMv, nJ, na, tb, cW, aal, Gv, ff, cr, kD, gl, dq, EZ, Z, jb, bMx, qa, gk, HP, bMA, gZ, Rm, ei, y, ra); #c1(HTR2B) c2(NP_000858) c3(5-hydroxytryptamine receptor 2B) c4(16603, 16604) c5(16605, 16606, 16607, 16608, 16609, 16610) c6( )c7(lD, T, fV, al, cz, v, ur, uu, wy, cd, BY, wC, ly, up, mQ, vo, lx, mg, lC, lB, ra, B); #c1(HTR2B) c2(XP_005246577) c3(5-hydroxytryptamine receptor 28 isoform X3) c4( )c5(267243) c6( )c7(lD, T, fV, al, cz, v, ur, uu, wy, cd, BY, wC, ly, up, mQ, vo, lx, mg, lC, lB, ra, B); #c1(HTR2B) c2(XP_011509375) c3(5-hydroxytryptamine receptor 28 isoform X2) c4( ) c5(299954, 299955, 299956, 299957, 299958) c6( )c7(lD, T, fV, al, cz, v, ur, uu, wy, cd, BY, wC, ly, up, ml, vo, lx, mg, lC, lB, ra, B); #c1(HTR2C) c2(NP_001243689) c3(5-hydroxytryptamine receptor 2C isoform a precursor) c4(91395) c5(91396, 91397, 91398, 91399, 91400, 91401, 91402, 91403, 91404, 91405, 91406) c6( )c7(bL, hl, bBC, bMD, jp, ob, e, bO, mS, bhL, bS, bMB, ct, ur, uu, ml, aj, v, il, rc, J, wy, HF, ww, bMG, mW, iX, sl, bMF, aql, ta, wP, jy, bK, K, an, mY, mV, he, bME, af, na, rz, mU, tb, aal, yL, bcD, gl, Eg, Z, cm, mZ, FT, mX, bJ, cv, hq, ok, bN, cA, bMA, ZT, Gs, bLi, vL, uJ, ei, lB, xg, mT); #c1(HTR2C) c2(NP_001243690) c3(5-hydroxytryptamine receptor 2C isoform b precursor) c4(91407) c5(91408, 91409, 91410, 91411, 91412) c6( )c7(bL, hl, bBC, bMD, jp, ob, e, bO, mS, bhL, bS, bMB, ct, ur, uu, ml, aj, v, il, rc, J, wy, HF, ww, bMG, mW, iX, sl, bMF, aql, ta, wP, jy, bK, K, an, mY, mV, he, bME, af, na, rz, mU, tb, aal, yL, bcD, gl, Eg, Z, cm, mZ, FT, mX, bJ, cv, hq, ok, bN, cA, bMA, ZT, Gs, bLi, vL, uJ, ei, lB, xg, mT); #c1 (HTR3A) c2(NP_000860) c3(5-hydroxytryptamine receptor 3A isoform b precursor) c4(16611, 16612, 16613) c5(16614, 16615, 16616, 16617, 16618, 16619, 16620, 16621, 16622, 16623, 16624, 16625, 16626, 16627, 16628, 16629, 16630) c6( )c7(T, jv, bcB, akd, bO, mS, bS, uu, ct, ml, ww, Ge, wy, mW, ix, Gb, aql, aT, bk, K, jj, rE, mY, mV, he, xa, na, hZ, mU, zQ, Gv, yL, Z, mZ, bN, mX, hq, cA, bMH, uJ, ra, pk, jd, kS, mT, qc); #c1(HTR3A) c2(NP_001155244) c3(5-hydroxytryptamine receptor 3A isoform c) c4(61542) c5(61543, 61544, 61545, 61546, 61547, 61548, 61549, 61550, 61551, 61552, 61553, 61554) c6( )c7(T, jv, bcB, akd, bO, mS, bS, uu, ct, ml, ww, Ge, wy, mW, ix, Gb, aql, aT, bK, K, jj, rE, mY, mV, he, xa, na, hZ, mU, zQ, Gv, yL, Z, mZ, bN, mX, hq, cA, bMH, uJ, ra, pk, jd, kS, mT, qc); #c1(HTR3A) c2(NP_998786) c3(5-hydroxytryptamine receptor 3A isoform a precursor) c4(265959, 265960, 265961) c5(265962, 265963, 265964, 265965, 265966, 265967, 265968, 265969, 265970, 265971, 265972, 265973) c6( )c7(T, jv, bcB, akd, bO, mS, bS, uu, ct, ml, ww, Ge, wy, mW, iX, Gb, aql, aT, bK, K, jj, rE, mY, mV, he, xa, na, hZ, mU, zQ, Gv, yL, Z, mZ, bN, mX, hq, cA, bMH, uJ, ra, pk, jd, kS, mT, qc); #c1(HTR3B) c2(NP_006019) c3(5-hydroxytryptamine receptor 38 precursor) c4(161211) c5(161212, 161213, 161214, 161215, 161216, 161217) c6( )c7(jv, bcB, bO, mS, bS, ct, wy, mW, iX, XY, Gb, aql, oj, aT, bK, mY, he, mV, mU, zQ, yL, Z, mZ, FT, mX, cA, jd, mT); #c1(HTR3B) c2(XP_011541365) c3(5-hydroxytryptamine receptor 38 isoform X1) c4(339967) c5(339968, 339969, 339970, 339971, 339972, 339973) c6( )c7(jv, bcB, bO, mS, bS, ct, wy, mW, iX, XY, Gb, aql, oj, aT, bK, mY, he, mV, mU, zQ, yL, Z, mZ, FT, mX, cA, jd, mT); #c1(HTR3B) c2(XP_011541368) c3(5-hydroxytryptamine receptor 3B isoform X4) c4(339974) c5(339975, 339976, 339977, 339978, 339979, 339980) c6( )c7(jv, bcB, bO, mS, bS, ct, wy, mW, iX, XY, Gb, aql, oj, aT, bK, mY, he, mV, mU, zQ, yL, Z, mZ, FT, mX, cA, jd, mT); #c1 (HTR3C) c2(NP_570126) c3(5-hydroxytryptamine receptor 3C precursor) c4(234007) c5(234008, 234009, 234010, 234011, 234012, 234013) c6( )c7(T, jv, aT, rE, bN, qc); #c1(HTR3D) c2(NP_001138615) c3(5-hydroxytryptamine receptor 3D isoform 1 precursor) c4(57183, 57184) c5(57185, 57186, 57187, 57188, 57189, 57190) c6( )c7(rE, jv, zQ, qc, aT); #c1(HTR3D) c2(NP_001157118) c3(5-hydroxytryptamine receptor 3D isoform 3 precursor) c4(62061) c5(62062, 62063, 62064, 62065, 62066, 62067) c6( )c7(rE, jv, zQ, qc, aT); #c1(HTR3D) c2(NP_872343) c3(5-hydroxytryptamine receptor 3D isoform 2) c4(256156) c5(256157, 256158, 256159, 256160, 256161, 256162, 256163, 256164, 256165, 256166, 256167, 256168) c6( )c7(rE, jv, zQ, qc, aT); #c1(HTR3E) c2(NP_001243542) c3(5-hydroxytryptamine receptor 3E isoform d precursor) c4(91000) c5(91001, 91002, 91003, 91004, 91005, 91006) c6( )c7(jv, zQ, aT, rE, ra, qc); #c1(HTR3E) c2(NP_001243543) c3(5-hydroxytryptamine receptor 3E isoform e) c4(91007) c5(91008, 91009, 91010, 91011, 91012, 91013) c6( )c7(jv, zQ, aT, rE, ra, qc); #c1(HTR3E) c2(NP_872395) c3(5-hydroxytryptamine receptor 3E isoform a) c4(256303) c5(256304, 256305, 256306, 256307, 256308, 256309) c6( )c7(jv, zQ, aT, rE, ra, qc); #c1(HTR3E) c2(NP_938055) c3(5-hydroxytryptamine receptor 3E isoform c precursor) c4(259578) c5(259579, 259580, 259581, 259582, 259583, 259584) c6( )c7(jv, zQ, aT, rE, ra, qc); #c1(HTR3E) c2(NP_938056) c3(5-hydroxytryptamine receptor 3E isoform b) c4(259585) c5(259586, 259587, 259588, 259589, 259590, 259591) c6( )c7(jv, zQ, aT, rE, ra, qc); #c1(HTR4) c2(NP_000861) c3(5-hydroxytryptamine receptor 4 isoform b) c4(16631, 16632) c5(16633, 16634, 16635, 16636, 16637, 16638, 16639, 16640, 16641, 16642, 16643, 16644) c6( )c7(T, ac, s, cA, wy, Jb, wL, afi, eZ, gZ, ti, Dp, Ja, bk, K, uu, at, ra, cd, bN, aa); #c1(HTR4) c2(NP_001035259) c3(5-hydroxytryptamine receptor 4 isoform a) c4(32701) c5(32702, 32703, 32704, 32705, 32706, 32707, 32708, 32709, 32710, 32711) c6( )c7(T, ac, s, cA, wy, Jb, wL, afi, eZ, gZ, ti, Dp, Ja, bk, K, uu, at, ra, cd, bN, aa); #c1(HTR4) c2(NP_001035262) c3(5-hydroxytryptamine receptor 4 isoform d) c4(32712) c5(32713, 32714, 32715, 32716, 32717, 32718) c6( )c7(T, ac, s, cA, wy, Jb, wL, afi, eZ, gZ, ti, Dp, Ja, bK, K, uu, at, ra, cd, bN, aa); #c1(HTR4) c2(NP_001035263) c3(5-hydroxytryptamine receptor 4 isoform i) c4(32719) c5(32720, 32721, 32722, 32723, 32724, 32725) c6( )c7(T, ac, s, cA, wy, Jb, wL, afi, eZ, gZ, ti, Dp, Ja, bK, K, uu, at, ra, cd, bN, aa); #c1(HTR4) c2(NP_001273339) c3(5-hydroxytryptamine receptor 4 isoform c) c4(104704) c5(104705, 104706, 104707, 104708, 104709, 104710) c6( )c7(T, ac, s, cA, wy, Jb, wL, afi, eZ, gZ, ti, Dp, Ja, bK, K, uu, at, ra, cd, bN, aa); #c1(HTR4) c2(NP_955525) c3(5-hydroxytryptamine receptor 4 isoform g) c4(261884) c5(261885, 261886, 261887, 261888, 261889, 261890, 261891, 261892, 261893, 261894, 261895, 261896, 261897, 261898) c6( )c7(T, ac, s, cA, wy, Jb, wL, afi, eZ, gZ, ti, Dp, Ja, bK, K, uu, at, ra, cd, bN, aa); #c1(HTR5A) c2(NP_076917) c3(5-hydroxytryptamine receptor 5A) c4(214213) c5(214214, 214215, 214216, 214217, 214218, 214219, 214220, 214221, 214222, 214223, 214224, 214225) c6( )c7(cA, Gs, bO, bS, uu, bK, K, wy, he, bN); #c1(HTR7) c2(NP_000863) c3(5-hydroxytryptamine receptor 7 isoform a) c4(16645) c5(16646, 16647, 16648, 16649, 16650, 16651) c6( ) c7(mS, bS, uu, ct, ci, wy, aR, xt, ot, mW, iX, oj, iv, bK, K, mY, he, mV, aQa, bEV, mU, bMl, Z, mZ, bN, mX, hq, dJ, ZT, Gs, HN, ra, mT); #c1(HTR7) c2(NP_062873) c3(5-hydroxytryptamine receptor 7 isoform d) c4(202194, 202195) c5(202196, 202197, 202198, 202199, 202200, 202201, 202202, 202203, 202204, 202205, 202206, 202207, 202208) c6( )c7(mS, bS, uu, ct, ci, wy, aR, xt, ot, mW, iX, oj, iv, bK, K, mY, he, mV, aQa, bEV, mU, bMl, Z, mZ, bN, mX, hq, dJ, ZT, Gs, HN, ra, mT); #c1(HTR7) c2(NP_062874) c3(5-hydroxytryptamine receptor 7 isoform b) c4(202209) c5(202210, 202211, 202212, 202213, 202214) c6( )c7(mS, bS, uu, ct, ci, wy, aR, xt, ot, mW, iX, oj, iv, bK, K, mY, he, mV, aQa, bEV, mU, bMl, Z, mZ, bN, mX, hq, dJ, ZT, Gs, HN, ra, mT); #c1(HTRA1) c2(NP_002766) c3(serine protease HTRA1 precursor) c4(131811, 131812, 131813) c5(131814, 131815, 131816, 131817, 131818, 131819, 131820, 131821, 131822, 131823, 131824, 131825, 131826, 131827, 131828, 131829, 131830, 131831) c6( )c7(bL, T, bMK, dD, NO, agC, aA, eb, n, eq, ai, aR, aJr, XX, av, ib, xE, aT, agD, dL, K, an, acd, db, Um, af, eL, bMJ, aW, Y, aSJ, aF, cl, t, uC, Uk, W, nb, vy, hL, m, EY, GF, y, it, iu); #c1(HTRA2) c2(NP_037379) c3(serine protease HTRA2, mitochondrial isoform 1 preproprotein) c4(175161) c5(175162, 175163, 175164, 175165, 175166, 175167) c6( )c7(T, wi, eX, bB, fr, ci, MR, kf, eV, K, an, zH, Y, vs, cl, t, xw, rR, c, bML, bE, U, hL, m, cB, X); #c1(HTRA2) c2(NP_659540) c3(serine protease HTRA2, mitochondrial isoform 2) c4(240288) c5(240289, 240290, 240291, 240292, 240293, 240294) c6( )c7(T, wi, eX, bB, fr, ci, MR, kf, eV, K, an, zH, Y, vs, cl, t, xw, rR, c, bML, bE, U, hL, m, cB, X); #c1(HTRA3) c2(NP_001284488) c3(serine protease HTRA3 isoform 2 precursor) c4(111905) c5(111906, 111907, 111908, 111909, 111910, 111911) c6( )c7(av, T, BJ, hL, K, m, cl, t, bz); #c1(HTRA3) c2(NP_444272) c3(serine protease HTRA3 isoform 1 precursor) c4(230222) c5(230223, 230224, 230225, 230226, 230227, 230228) c6( )c7(av, T, BJ, hL, K, m, cL, t, bz); #c1(HTRA4) c2(NP_710159) c3(serine protease HTRA4 precursor) c4(246517) c5(246518, 246519, 246520, 246521, 246522, 246523) c6( )c7(jv, eb); #c1(HTT) c2(NP_002102) c3(huntingtin) c4( )c5(124585, 124586, 124587, 124588, 124589, 124590) c6( )c7(tJ, T, mr, ahc, bMN, bmk, nx, bMM, e, bO, eX, amj, bS, gf, aks, xg, bfD, cd, aR, ad, Hr, PW, mj, dE, aT, HG, lG, cy, eV, yZ, K, an, rE, he, on, af, li, aW, yL, QP, Rl, hf, cm, xw, dr, aeb, hq, ck, bN, rR, bb, KB, Kv, U, gZ, Kj, Lz, aen, qc); #c1(HUNK) c2(NP_055401) c3(hormonally up-regulated neu tumor-associated kinase) c4(180837) c5(180838, 180839, 180840, 180841, 180842, 180843) c6( )c7(bL, aT, qj, g, aot, cm, Ro); #c1(HUNK) c2(XP_011527839) c3(hormonally up-regulated neu tumor-associated kinase isoform X1) c4( )c5(322778, 322779) c6( ) c7(bL, aT, qj, g, aot, cm, Ro); #c1(HUS1) c2(NP_004498) c3(checkpoint protein HUS1) c4(146944, 146945) c5(146946, 146947, 146948, 146949, 146950, 146951, 146952, 146953, 146954, 146955, 146956, 146957, 146958, 146959, 146960, 146961, 146962, 146963, 146964, 146965) c6( )c7(nM, c, aT, at, dL, ci, bX); #c1(HUS1B) c2(NP_683762) c3(checkpoint protein HUS1B) c4(242565) c5(242566, 242567, 242568, 242569, 242570, 242571) c6( )c7(cm); #c1(HUWE1) c2(XP_005262022) c3(E3 ubiquitin-protein ligase HUWE1 isoform X1) c4(277698, 277699) c5(277700, 277701, 277702, 277703, 277704, 277705, 277706, 277707, 277708, 277709, 277710, 277711, 277712, 277713, 277714, 277715, 277716, 277717, 277718, 277719) c6( )c7(es, bMO, bLM, nf, h, lo, fr, nA, oU); #c1(HUWE1) c2(XP_011529053) c3(E3 ubiquitin-protein ligase HUWE1 isoform X2) c4(324405) c5(324406, 324407, 324408, 324409, 324410, 324411, 324412, 324413, 324414, 324415, 324416, 324417, 324418, 324419, 324420, 324421, 324422, 324423, 324424, 324425, 324426, 324427) c6( )c7(es, bMO, bLM, nf, h, lo, fr, nA, oU); #c1(HUWE1) c2(XP_011529055) c3(E3 ubiquitin-protein ligase HUWE1 isoform X4) c4(324428, 324429) c5(324430, 324431, 324432, 324433, 324434, 324435) c6( )c7(es, bMO, bLM, nf, h, lo, fr, nA, oU); #c1(HUWE1) c2(XP_011529059) c3(E3 ubiquitin-protein ligase HUWE1 isoform X8) c4(324436) c5( )c6( )c7(es, bMO, bLM, nf, h, lo, fr, nA, oU); #c1(HUWE1) c2(XP_011529060) c3(E3 ubiquitin-protein ligase HUWEL isoform X9) c4(324437, 324438) c5(324439, 324440, 324441, 324442, 324443, 324444, 324445, 324446) c6( )c7(es, bMO, bLM, nf, h, lo, fr, nA, oU); #c1(HVCN1) c2(XP_011537150) c3(voltage-gated hydrogen channel 1 isoform X1) c4(334899, 334900) c5(334901, 334902, 334903, 334904, 334905, 334906, 334907, 334908, 334909, 334910, 334911, 334912, 334913, 334914, 334915, 334916) c6( )c7(eF, uE); #c1(HYAL1) c2(NP_149349) c3(hyaluronidase-1 isoform 1 precursor) c4(227435, 227436, 227437, 227438) c5(227439, 227440, 227441, 227442, 227443, 227444, 227445, 227446, 227447, 227448, 227449, 227450, 227451, 227452, 227453, 227454, 227455, 227456, 227457, 227458, 227459) c6( )c7(T, bMP, n, cL, ci, Jx, eA, o, ad, av, ft, aT, dL, ae, eL, aW, ff, en, cl, t, ik, go, Lq, m, lC); #c1(HYAL1) c2(NP_695014) c3(hyaluronidase-1 isoform 2 precursor) c4(245672) c5(245673, 245674, 245675, 245676, 245677, 245678, 245679, 245680, 245681, 245682, 245683, 245684, 245685, 245686, 245687, 245688, 245689, 245690) c6( )c7(T, bMP, n, cL, ci, Jx, eA, o, ad, av, ft, aT, dL, ae, eL, aW, ff, en, cl, t, ik, go, Lq, m, lC); #c1(HYAL1) c2(NP_695015) c3(hyaluronidase-1 isoform 3) c4( )c5(245691, 245692, 245693, 245694, 245695, 245696) c6( )c7(T, bMP, n, cL, ci, Jx, eA, o, ad, av, ft, aT, dL, ae, eL, aW, ff, en, cl, t, ik, go, Lq, m, lC); #c1(HYAL1) c2(NP_695017) c3(hyaluronidase-1 isoform 5) c4(245697) c5(245698, 245699, 245700, 245701, 245702) c6( )c7(T, bMP, n, cL, ci, Jx, eA, o, ad, av, ft, aT, dL, ae, eL, aW, ff, en, cL, t, iK, go, Lq, m, lC); #c1(HYAL2) c2(XP_005265582) c3(hyaluronidase-2 isoform X1) c4(279594, 279595, 279596, 279597, 279598, 279599) c5(279600, 279601, 279602, 279603, 279604, 279605, 279606, 279607, 279608, 279609, 279610, 279611, 279612, 279613, 279614, 279615, 279616, 279617, 279618, 279619, 279620, 279621, 279622, 279623, 279624, 279625, 279626, 279627, 279628, 279629, 279630, 279631, 279632, 279633, 279634, 279635, 279636, 279637, 279638, 279639, 279640, 279641, 279642, 279643, 279644, 279645, 279646, 279647, 279648, 279649, 279650, 279651, 279652, 279653) c6( )c7(lD, T, fM, dA, iW, bMP, jh, eA, BY, av, ft, aT, d, iE, aW, ff, en, g, cl, bz, DL, Dy, go, lC); #c1(HYAL3) c2(NP_001186959) c3(hyaluronidase-3 isoform 2 precursor) c4(78874, 78875) c5(78876, 78877, 78878, 78879, 78880, 78881, 78882, 78883, 78884, 78885, 78886, 78887, 78888, 78889, 78890, 78891, 78892, 78893, 78894) c6( ) c7(T, ft, eA, hL, cl); #c1(HYAL3) c2(NP_001186960) c3(hyaluronidase-3 isoform 3) c4(78895) c5(78896, 78897, 78898, 78899, 78900, 78901) c6( )c7(T, ft, eA, hL, cl); #c1(HYAL3) c2(NP_001186961) c3(hyaluronidase-3 isoform 4) c4( )c5(78902, 78903, 78904, 78905, 78906, 78907) c6( )c7(T, ft, eA, hL, cl); #c1(HYAL3) c2(NP_003540) c3(hyaluronidase-3 isoform 1 precursor) c4(138980, 138981) c5(138982, 138983, 138984, 138985, 138986, 138987) c6( )c7(T, ft, eA, hL, cl); #c1(HYAL4) c2(XP_011514292) c3(hyaluronidase-4 isoform X1) c4(306917, 306918, 306919) c5(306920, 306921, 306922, 306923, 306924, 306925, 306926, 306927, 306928, 306929) c6( )c7(go, Uc); #c1(HYAL4) c2(XP_011514294) c3(hyaluronidase-4 isoform X3) c4( )c5(306930, 306931, 306932, 306933, 306934, 306935, 306936, 306937, 306938, 306939, 306940, 306941) c6( )c7(go, Uc); #c1(HYD1N) c2(NP_001185471) c3 (hydrocephalus-inducing protein homolog isoform c) c4( )c5(75691, 75692, 75693, 75694, 75695, 75696) c6( )c7(bMQ, ul, Pf, MG, bN); #c1(HYD1N) c2(NP_001185472) c3(hydrocephalus-inducing protein homolog isoform d) c4(75697) c5(75698, 75699, 75700, 75701, 75702, 75703, 75704) c6( )c7(bMQ, ul, Pf, MG, bN); #c1(HYD1N) c2(NP_001257903) c3(hydrocephalus-inducing protein homolog isoform a) c4(95737, 95738, 95739, 95740, 95741) c5(95742, 95743, 95744, 95745, 95746, 95747, 95748, 95749, 95750, 95751, 95752, 95753, 95754, 95755, 95756, 95757, 95758, 95759, 95760, 95761, 95762, 95763, 95764, 95765, 95766, 95767, 95768) c6( )c7(bMQ, ul, Pf, MG, bN); #c1(HYD1N) c2(NP_060028) c3(hydrocephalus-inducing protein homolog isoform b) c4( )c5(193773, 193774, 193775, 193776, 193777, 193778, 193779, 193780) c6( )c7(bMQ, ul, Pf, MG, bN); #c1(HYD1N) c2(XP_006721269) c3(hydrocephalus-inducing protein homolog isoform X5) c4(293905) c5(293906, 293907, 293908, 293909) c6( )c7(bMQ, ul, Pf, MG, bN); #c1(HYD1N) c2(XP_011521448) c3(hydrocephalus-inducing protein homolog isoform X1) c4( )c5(316221, 316222, 316223, 316224, 316225) c6( )c7(bMQ, ul, Pf, MG, bN); #c1 (HYD1N) c2(XP_011521453) c3(hydrocephalus-inducing protein homolog isoform X4) c4(316226, 316227) c5(316228) c6( )c7(bMQ, ul, Pf, MG, bN); #c1(HYKK) c2(NP_001013641) c3(hydroxylysine kinase isoform 1) c4(25630, 25631, 25632) c5(25633, 25634, 25635, 25636, 25637, 25638) c6( )c7(av, bMS, at, bMR, bz, iu); #c1(HYKK) c2(NP_001077081) c3(hydroxylysine kinase isoform 2) c4( ) c5(37680, 37681, 37682, 37683, 37684, 37685) c6( )c7(av, bMS, at, bMR, bz, iu); #c1(HYKK) c2(XP_011519533) c3 (hydroxylysine kinase isoform X1) c4( )c5(314185, 314186, 314187, 314188, 314189, 314190) c6( )c7(av, bMS, at, bMR, bz, iu); #c1(HYLS1) c2(NP_659451) c3(hydrolethalus syndrome protein 1) c4(239978) c5(239979, 239980, 239981, 239982, 239983, 239984, 239985, 239986, 239987, 239988, 239989, 239990, 239991, 239992) c6( )c7(n, bMT, VB, ben); #c1(HYOU1) c2(NP_001124463) c3(hypoxia up-regulated protein I precursor) c4(49069, 49070) c5(49071, 49072, 49073, 49074, 49075) c6( )c7(bL, T, akN, d, ns, cy, aj, v, j, K, aF, uL, ae, eA, J, ad); #c1(HYOU1) c2(XP_005271450) c3(hypoxia up-regulated protein 1 isoform X3) c4(283404, 283405, 283406, 283407) c5(283408, 283409, 283410, 283411, 283412, 283413, 283414, 283415, 283416, 283417, 283418, 283419, 283420, 283421, 283422, 283423) c6( ) c7(bL, T, akN, d, ns, cy, aj, v, j, K, aF, uL, ae, eA, J, ad); #c1(HYOU1) c2(XP_011540859) c3(hypoxia up-regulated protein 1 isoform X1) c4( ) c5(339374) c6( )c7(bL, T, akN, d, ns, cy, aj, v, j, K, aF, uL, ae, eA, J, ad); #c1(HYOU1) c2(XP_011540860) c3(hypoxia up-regulated protein I isoform X2) c4(339375) c5(339376, 339377, 339378, 339379, 339380, 339381, 339382, 339383, 339384, 339385, 339386, 339387, 339388, 339389, 339390, 339391, 339392, 339393, 339394, 339395) c6( )c7(bL, T, akN, d, ns, cy, aj, v, j, K, aF, uL, ae, eA, J, ad); #c1(HYPM) c2(NP_036406) c3(huntingtin-interacting protein M) c4(173629) c5(173630, 173631, 173632, 173633, 173634, 173635) c6( )c7(c); #c1(IAPP) c2(NP_000406) c3(islet amyloid polypeptide precursor) c4(8593, 8594) c5(8595, 8596, 8597, 8598, 8599, 8600, 8601, 8602, 8603, 8604, 8605, 8606, 8607, 8608, 8609, 8610) c6( )c7(bL, T, dt, fM, eP, Zz, mr, eR, if, ha, eX, fd, aA, Bm, n, akA, cz, dG, ci, aj, PJ, v, vm, qN, q, J, ad, ai, XX, mj, aE, hW, nv, Vr, pX, dv, aT, kf, vH, blg, dL, eV, K, bq, H, ae, dl, eb, cp, eL, A, d, MD, nO, aW, Y, hl, r, aq, aF, jh, apm, iR, bw, iK, bz, bJ, LB, ck, kP, eM, iT, bE, HN, mg, cj, hR, mf, R, Lz, bt, B); #c1(IARS) c2(NP_038203) c3(isoleucine-tRNA ligase, cytoplasmic) c4(176273, 176274, 176275, 176276, 176277, 176278, 176279, 176280) c5(176281, 176282, 176283, 176284, 176285, 176286, 176287, 176288, 176289, 176290, 176291, 176292, 176293, 176294, 176295, 176296, 176297, 176298, 176299, 176300, 176301, 176302, 176303, 176304, 176305, 176306, 176307, 176308, 176309, 176310, 176311, 176312, 176313, 176314, 176315, 176316, 176317, 176318, 176319, 176320, 176321, 176322, 176323, 176324, 176325, 176326, 176327, 176328, 176329, 176330, 176331, 176332, 176333, 176334, 176335, 176336, 176337, 176338, 176339, 176340) c6( )c7(bL, gh, T, wu, aZ, aj, v, ci, J, ad, kH, Ne, aT, vc, K, Dz, wm, ae, je, Qr, aW, dq, lo, aq, iM, B); #c1(IARS2) c2(NP_060530) c3(isoleucine-RNA ligase, mitochondrial precursor) c4(196414, 196415) c5(196416, 196417, 196418, 196419, 196420, 196421, 196422, 196423, 196424, 196425, 196426, 196427) c6( )c7(bL, vc, ajU, ey, K); #c1(IBA57) c2(NP_001010867) c3(putative transferase CAF17, mitochondrial precursor) c4(23854, 23855) c5(23856, 23857, 23858, 23859, 23860, 23861, 23862) c6( )c7(hb, v, oj, bMU); #c1(IBSP) c2(NP_004958) c3(bone sialoprotein 2 precursor) c4(151458) c5(151459, 151460, 151461, 151462, 151463, 151464) c6( )c7(gh, T, zt, if, fd, jE, ci, qN, eA, ad, Bl, aE, aT, dL, ae, eL, bA, aW, Y, hC, ff, kp, en, g, t, bz, fs, D, eM, m, art, bwg, y); #c1(IBTK) c2(NP_056340) c3(inhibitor of Bruton tyrosine kinase isoform 1) c4(187158) c5(187159, 187160, 187161, 187162, 187163, 187164) c6( )c7(j); #c1(ICA1) c2(NP_001263407) c3(islet cell autoantigen 1 isoform b) c4(97264) c5(97265, 97266, 97267, 97268, 97269, 97270, 97271, 97272, 97273, 97274, 97275, 97276) c6( )c7(y, A, J, bMV, eZ, hD, fe, aj, qj, dw, cm, V, ae, ad, yF); #c1(ICA1) c2(NP_004959) c3(islet cell autoantigen 1 isoform a) c4(151465) c5(151466, 151467, 151468, 151469, 151470, 151471, 151472, 151473, 151474, 151475, 151476, 151477, 151478, 151479, 151480, 151481, 151482, 151483, 151484, 151485, 151486, 151487, 151488, 151489, 151490, 151491) c6( )c7 (y, A, J, bMV, eZ, hD, fe, aj, qj, dw, cm, V, ae, ad, yF); #c1(ICA1) c2(XP_011513651) c3 (islet cell autoantigen 1 isoform X2) c4(305954, 305955, 305956) c5(305957, 305958, 305959, 305960, 305961, 305962) c6( )c7(y, A, J, bMV, eZ, hD, fe, aj, qj, dw, cm, V, ae, ad, yF); #c1(ICA1) c2(XP_01513655) c3(islet cell autoantigen 1 isoform X3) c4(305963) c5( )c6( )c7(y, A, J, bMV, eZ, HD, fe, aj, qj, dw, cm, V, ae, ad, yF); #c1(ICAM1) c2(NP_000192) c3(intercellular adhesion molecule 1 precursor) c4(4271, 4272) c5(4273, 4274, 4275, 4276, 4277, 4278, 4279, 4280, 4281, 4282, 4283, 4284) c6( )c7(bM, T, aC, fR, bJW, ea, e, al, if, bMY, fd, bB, aeu, cL, fo, qj, ci, lR, mF, aY, bMW, Hr, wa, x, xy, dY, aE, Bc, M, dV, Ds, K, hu, wt, hH, pk, ass, bc, eL, cp, hm, eN, aW, az, hl, apn, mQ, cT, hN, vs, VE, La, g, as, hJ, eE, bwe, uC, eF, ir, cm, iP, qJ, bw, yl, eM, nB, iA, aau, iT, wE, U, cj, vy, Zn, mk, k, Hb, m, hh, fy, aFf, aDi, NN, qR, ajJ, eg, iu, cZ, dD, aFG, My, yG, ht, fN, aw, n, BH, aj, fr, ct, uE, pM, qN, qW, ai, eh, El, ke, ft, no, Wy, ec, pe, cc, kl, kH, aEB, lG, cU, aal, dL, wb, ux, dd, ey, vx, aDp, wo, aqi, A, sp, EU, fJ, Ff, ge, fe, aq, aGq, cM, ar, kT, iO, RB, W, Pa, bP, dB, hk, GC, wk, JM, at, E, apY, uz, va, eo, Pr, B, fl, ayV, aMk, L, bL, Dw, fM, eR, DD, fG, yk, jx, rf, aA, cR, cz, fF, hy, j, v, C, ze, bX, hz, V, pd, q, J, eA, zT, mP, nv, nX, jZ, yg, aT, yu, cy, jj, bq, aFm, ae, af, eu, bA, wr, iU, nO, aOK, oV, Y, aBq, r, afq, lo, aDm, en, wA, t, pb, ck, sS, OW, Dy, c, ac, bMZ, aQd, hc, nM, iM, iQ, lV, hO, mf, rk, dy, fD, bt, ao, hS, hl, kh, wi, qb, dM, df, axd, jN, rl, iW, jh, auQ, bS, dG, jE, eq, ad, hX, bwf, dP, hd, ib, h, ds, oW, LA, hD, vc, JK, ji, jo, NM, vO, yF, sj, uQ, uX, d, BN, lR, je, cH, cW, KS, eH, ff, cr, rM, dN, anQ, eZ, aF, hp, S, Fq, iV, Z, iR, BG, bz, DF, ek, TA, es, D, gk, Bv, bMX, zr, axs, yE, bE, atM, amS, X, aDB, aS, XO, ei, od, GF, y, vt, ne, hv, pC); #c1(ICAM2) c2(NP_001093259) c3(intercellular adhesion molecule 2 precursor) c4(40209) c5(40210, 40211, 40212, 40213, 40214, 40215, 40216, 40217, 40218, 40219, 40220, 40221, 40222, 40223, 40224, 40225, 40226, 40227, 40228, 40229, 40230, 40231) c6( )c7(T, aE, Cj, ff, bm, aA, h, fd, aF, en, iV, XB); #c1(ICAM3) c2(NP_002153) c3(intercellular adhesion molecule 3 precursor) c4(125991) c5(125992, 125993, 125994, 125995, 125996, 125997, 125998, 125999) c6( )c7(xR, if, ht, jx, wc, jh, h, qN, hu, y, bc); #c1(ICAM4) c2(NP_001034221) c3(intercellular adhesion molecule 4 isoform 3 precursor) c4(31606) c5(31607, 31608, 31609, 31610, 31611, 31612) c6( )c7(bL, qb, aW, bNa, jx, eZ, aT, fd, ta, ae, ad); #c1(ICAM4) c2(NP_001535) c3(intercellular adhesion molecule 4 isoform 1 precursor) c4(118680) c5(118681, 118682, 118683, 118684, 118685, 118686, 118687) c6( )c7(bL, qb, aW, bNa, jx, eZ, aT, fd, ta, ae, ad); #c1(ICAM4) c2(NP_07/772) c3 (intercellular adhesion molecule 4 isoform 2 precursor) c4(212036) c5(212037, 212038, 212039, 212040, 212041) c6( ) c7(bL, qb, aW, bNa, jx, eZ, aT, fd, ta, ae, ad); #c1(ICAM5) c2(NP_003250) c3(intercellular adhesion molecule 5 precursor) c4(136820) c5(136821, 136822, 136823, 136824, 136825, 136826) c6( )c7(T, aW, ff, jx, qK, eZ, aT, fd, hy, ci, en, ae, ad); #c1(ICE1) c2(NP_056140) c3(little elongation complex subunit 1) c4(185909) c5(185910, 185911, 185912, 185913, 185914, 185915) c6( )c7(j, cz); #c1(ICE2) c2(NP_001263314) c3(little elongation complex subunit 2 isoform c) c4( )c5(97175, 97176, 97177, 97178, 97179, 97180, 97181, 97182, 97183, 97184, 97185, 97186, 97187, 97188, 97189, 97190, 97191, 97192, 97193, 97194, 97195, 97196, 97197, 97198) c6( )c7(cu); #c1(ICE2) c2(NP_078887) c3(little elongation complex subunit 2 isoform a) c4(215915, 215916, 215917, 215918, 215919, 215920) c5(215921, 215922, 215923, 215924, 215925, 215926, 215927, 215928, 215929, 215930, 215931, 215932) c6( )c7(cu); #c1(ICK) c2(NP_057597) c3(serine/threonine-protein kinase ICK) c4(191547) c5(191548, 191549, 191550, 191551, 191552, 191553, 191554, 191555, 191556, 191557) c6( )c7(T, jh, fd, bB, n, eA, eW, x, aE, kH, h, aT, K, bNb, Uf, d, Y, alJ, kp, bz, dB, bE, cj, k, MC, bt); #c1(ICMT) c2(XP_011539442) c3(protein-S-isoprenylcysteine O-methyltransferase isoform X1) c4( )c5(337612, 337613, 337614, 337615, 337616) c6( )c7(T, lp, eH, kp, ey, ei, eF); #c1(ICOS) c2(NP_036224) c3(inducible T-cell costimulator precursor) c4(172439, 172440) c5(172441, 172442, 172443, 172444, 172445, 172446, 172447, 172448, 172449, 172450, 172451, 172452) c6( )c7(bM, T, dD, dA, fR, aEj, ea, iW, jx, jh, wV, oH, aFF, bX, eq, Jx, qN, abC, V, mC, qW, Oe, xy, afm, qh, aT, hD, ji, bc, yF, sj, qw, hM, A, Cw, aW, sp, bNc, Oj, Y, lC, aw, mQ, eZ, lo, hp, eE, jT, cM, Wt, lY, cT, W, hr, cj, fL, y, zA, cn); #c1(ICOSLG) c2(NP_001269979) c3(ICOS ligand isoform b precursor) c4(103342) c5(103343, 103344, 103345, 103346, 103347) c6( )c7(Kd, bM, aA, wV, eA, cF, Dq, Nr, A, Y, ge, fe, po, aF, hp, iV, aaO, Wt, Nq, ck, Bv, OW, y, bt); #c1(ICOSLG) c2(NP_001269980) c3(ICOS ligand isoform c precursor) c4(103348) c5(103349, 103350, 103351, 103352, 103353, 103354) c6( )c7(Kd, bM, aA, wV, eA, cF, Dq, Nr, A, Y, ge, fe, po, aF, hp, iV, aaO, Wt, Nq, ck, Bv, OW, y, bt); #c1(ID1) c2(NP_002156) c3(DNA-binding protein inhibitor ID-1 isoform a) c4(126039) c5(126040, 126041, 126042, 126043, 126044, 126045, 126046) c6( )c7(bL, gh, T, aC, fG, if, ht, aA, cz, fd, j, ci, qN, zP, AO, eA, ad, eJ, hd, aE, nv, qK, aT, hD, dL, jj, bq, ae, eL, aW, Y, ff, cr, aF, yq, en, g, hJ, t, bz, ck, Qi, nM, eM, nB, dy, m, lC, iu); #c1(ID1) c2(NP_851998) c3(DNA-binding protein inhibitor lD-l isoform b) c4(254769) c5(254770, 254771, 254772, 254773, 254774, 254775) c6( )c7(bL, gh, T, aC, fG, if, ht, aA, cz, fd, j, ci, qN, zP, AO, eA, ad, eJ, hd, aE, nv, qK, aT, hD, dL, jj, bq, ae, eL, aW, Y, ff, cr, aF, yq, en, g, hJ, t, bz, ck, Di, nM, eM, nB, dy, m, lC, iu); #c1(ID2) c2(NP_002157) c3(DNA-binding protein inhibitor ID-2) c4(126047) c5(126048, 126049, 126050, 126051, 126052, 126053, 126054, 126055, 126056, 126057, 126058, 126059, 126060, 126061, 126062, 126063) c6( )c7(T, fM, eR, ex, cz, fd, hz, ci, aY, tK, eA, ad, av, ju, eV, lQ, jj, dQ, ae, A, ff, fQ, sm, en, t, cm, Qi, bP, bE, vy, m, Pk, bt, B); #c1(ID4) c2(NP_001537) c3(DNA-binding protein inhibitor ID-4) c4(118688) c5(118689, 118690, 118691, 118692, 118693, 118694, 118695) c6( )c7(T, auW, oe, jh, aA, n, ci, zP, q, ad, eA, yt, aT, dL, lQ, ae, eL, bA, zQ, d, bNd, aW, ff, cr, r, aF, en, g, t, ck, Qi, dB, cj, m, k, bt); #c1(IDE) c2(NP_001159418) c3(insulin-degrading enzyme isoform 2) c4(63984) c5(63985, 63986, 63987, 63988, 63989) c6( )c7(bL, aZ, cz, ti, aj, dG, v, fK, J, jy, an, H, af, kl, A, cm, t, mg, mf, R, ei, L); #c1(IDE) c2(NP_004960) c3(insulin-degrading enzyme isoform 1) c4(151492) c5(151493, 151494, 151495, 151496, 151497, 151498, 151499) c6( )c7(bL, aZ, cz, ti, aj, dG, v, fK, J, jy, an, H, af, kl, A, cm, t, mg, mf, R, ei, L); #c1(IDE) c2(XP_005269823) c3(insulin-degrading enzyme isoform X1) c4(282891) c5( )c6( )c7(bL, aZ, cz, ti, aj, dG, v, fK, J, jy, an, H, af, kl, A, cm, t, mg, mf, R, ei, L); #c1(IDE) c2(XP_005269826) c3(insulin-degrading enzyme isoform X2) c4( )c5(282892, 282893, 282894, 282895, 282896, 282897) c6( ) c7(bL, aZ, cz, ti, aj, dG, v, fK, J, jy, an, H, af, kl, A, cm, t, mg, mf, R, ei, L); #c1(IDH1) c2(NP_001269315) c3(isocitrate dehydrogenase [NADP] cytoplasmic) c4(101082, 101083, 101084, 101085) c5(101086, 101087, 101088, 101089, 101090, 101091, 101092, 101093, 101094, 101095, 101096, 101097, 101098, 101099, 101100, 101101, 101102, 101103, 101104, 101105, 101106, 101107, 101108, 101109, 101110, 101111, 101112, 101113, 101114, 101115, 101116, 101117) c6( )c7(T, aC, bB, ci, bNe, x, ajv, K, eG, aW, nY, g, as, eF, bw, aHd, bEu, qJ, eM, nB, iT, k, ym, bcW, Wx, Uc, qy, n, oY, br, Mo, jz, h, he, Lg, fe, aq, pm, cM, fs, Yr, auC, oU, bNg, aul, dB, hk, ajn, pg, anx, WL, B, bzl, q, eA, aT, dz, jj, bcV, jf, Uf, iU, Y, auc, bEc, r, qL, gm, ck, QG, nM, im, bt, On, Mb, kh, iW, ha, bkL, jh, auq, hF, Fc, bNf, ad, Jm, av, iC, hd, fx, Rq, Db, Ot, d, aJK, dq, Vl, iV, iR, Hp, pc, bE, blu, Fk, jr); #c1(IDH2) c2(NP_001276839) c3(isocitrate dehydrogenase [NADP], mitochondrial isoform 2) c4( )c5(108452, 108453, 108454, 108455, 108456, 108457) c6( )c7(nZ, bcU, T, kh, dD, Mb, ea, e, qy, jh, n, oY, eq, ci, hF, Fc, eA, br, Jm, av, Mo, hd, jz, x, cc, Ot, dz, K, j, bcV, aJu, Lg, bA, Uf, d, aU, iU, Y, auc, xs, aRO, bEc, nY, fe, dq, aq, gm, Vl, g, as, eF, iR, cM, fs, Yr, nC, pm, Hp, ck, bw, pc, GU, nB, dB, hk, iT, blu, mx, pg, Fk, k, anx, bt, bcW, B, jr); #c1(IDH2) c2(NP_001277043) c3(isocitrate dehydrogenase [NADP], mitochondrial isoform 3) c4( ) c5(108991, 108992, 108993, 108994, 108995) c6( )c7(nZ, bcU, T, kh, dD, Mb, ea, e, qy, jh, n, oY, eq, ci, hF, Fc, eA, br, Jm, av, Mo, hd, jz, x, cc, Ot, dz, K, j, bcV, aJu, Lg, bA, Uf, d, alJ, iU, Y, auc, xs, aRO, bEc, nY, fe, dq, aq, gm, Vl, g, as, eF, iR, cM, fs, Yr, nC, pm, Hp, ck, bw, pc, GU, nB, dB, hk, iT, blu, mx, pg, Fk, k, anx, bt, bcW, B, jr); #c1(IDH2) c2(NP_002159) c3(isocitrate dehydrogenase [NADP], mitochondrial isoform 1 precursor) c4(126064, 126065, 126066) c5(126067, 126068, 126069, 126070, 126071, 126072, 126073) c6( )c7(nZ, bcU, T, kh, dD, Mb, ea, e, qy, jh, n, oY, eq, ci, hF, Fc, eA, br, Jm, av, Mo, hd, jz, x, cc, Ot, dz, K, jj, bcV, aJu, Lg, bA, Uf, d, alJ, iU, Y, auc, xs, aRO, bEc, nY, fe, dq, aq, gm, Vl, g, as, eF, iR, cM, fs, Yr, nC, pm, Hp, ck, bw, pc, GU, nB, dB, hk, iT, blu, mx, pg, Fk, k, anx, bt, bcW, B, jr); #c1(IDH3B) c2(NP_001245313) c3(isocitrate dehydrogenase [NAD] subunit beta, mitochondrial isoform d) c4( )c5(93103, 93104, 93105, 93106, 93107, 93108) c6( )c7(nC, bNh, aA); #c1(IDH3B) c2(NP_008830) c3(isocitrate dehydrogenase [NAD] subunit beta, mitochondrial isoform a precursor) c4(168888) c5(168889, 168890, 168891, 168892, 168893, 168894, 168895, 168896, 168897, 168898, 168899) c6( )c7(nC, bNh, aA); #c1(IDH3B) c2(NP_777280) c3(isocitrate dehydrogenase [NAD] subunit beta, mitochondrial isoform b precursor) c4(251080) c5(251081, 251082, 251083, 251084, 251085, 251086) c6( )c7(nC, bNh, aA); #c1(IDH3B) c2(XP_005260773) c3(isocitrate dehydrogenase [NAD] subunit beta, mitochondrial isoform X1) c4(276868, 276869) c5(276870) c6( )c7(nC, bNh, aA); #c1(IDNK) c2(NP_001001551) c3(probable gluconokinase isoform 1) c4(18677, 18678, 18679) c5(18680, 18681, 18682, 18683, 18684, 18685, 18686, 18687, 18688, 18689, 18690, 18691) c6( )c7(cz); #c1(IDNK) c2(XP_006717173) c3(probable gluconokinase isoform X3) c4(290784) c5(290785, 290786, 290787, 290788, 290789, 290790) c6( )c7(cz); #c1(IDNK) c2(XP_0067/7174) c3(probable gluconokinase isoform X2) c4( )c5(290791) c6( )c7(cz); #c1(IDNK) c2(XP_011517002) c3(probable gluconokinase isoform X1) c4( )c5(310487, 310488, 310489, 310490, 310491) c6( )c7(cz); #c1(ID01) c2(NP_002155) c3(indoleamine 2,3-dioxygenase 1) c4(126007, 126008, 126009, 126010) c5(126011, 126012, 126013, 126014, 126015, 126016, 126017, 126018, 126019, 126020, 126021, 126022, 126023, 126024, 126025, 126026, 126027, 126028, 126029, 126030, 126031, 126032, 126033, 126034, 126035, 126036, 126037, 126038) c6( )c7(T, fR, aPJ, iW, jx, eb, bB, bS, gf, ct, fr, kO, pK, V, eh, dP, hd, F, xE, ds, aT, h, ji, K, bc, aW, az, fe, CQ, iV, Z, t, uC, bz, iP, bJz, aDN, es, iA, nB, bE, NL, go, U, bko, m, xh, fy, bt, it, B, y); #c1(IDO2) c2(NP_919270) c3(indoleamine 2,3-dioxygenase 2) c4(258382, 258383) c5(258384, 258385, 258386, 258387, 258388, 258389, 258390, 258391, 258392, 258393, 258394, 258395) c6( )c7(T, aC, fr, aE, BA); #c1(IDS) c2(NP_000193) c3(iduronate 2-sulfatase isoform a preproprotein) c4(4285, 4286, 4287) c5(4288, 4289, 4290, 4291, 4292, 4293, 4294, 4295, 4296, 4297) c6(4298, 4299, 4300) c7(cZ, wM, afm, kg, bNi, aW, xz, bmE, cV, aT, dr, n, agG, gf, Lq, ade, Ku, dd, EZ, ajM, jr); #c1(IDS) c2(NP_001160022) c3(iduronate 2-sulfatase isoform c) c4( )c5(65036, 65037, 65038, 65039, 65040, 65041, 65042, 65043, 65044, 65045, 65046, 65047, 65048, 65049, 65050, 65051, 65052) c6(65053, 65054, 65055, 65056) c7(cZ, wM, afm, kg, bNi, aW, xz, bmE, cV, aT, dr, n, agG, gf, Lq, ade, Ku, dd, EZ, ajM, jr); #c1(IDS) c2(NP_006114) c3(iduronate 2-sulfatase isoform b precursor) c4(162145, 162146, 162147, 162148) c5(162149, 162150, 162151, 162152, 162153, 162154, 162155, 162156, 162157, 162158, 162159, 162160, 162161, 162162, 162163, 162164, 162165, 162166, 162167, 162168, 162169, 162170, 162171, 162172, 162173, 162174, 162175, 162176, 162177, 162178, 162179, 162180) c6(162181, 162182, 162183, 162184, 162185, 162186, 162187) c7(cZ, wM, afm, kg, bNi, aW, xz, bmE, cV, aT, dr, n, agG, gf, Lq, ade, Ku, dd, EZ, ajM, jr); #c1(IDUA) c2(NP_000194) c3(alpha-L-iduronidase precursor) c4(4301) c5(4302, 4303, 4304, 4305, 4306, 4307, 4308, 4309, 4310, 4311, 4312, 4313, 4314, 4315, 4316, 4317, 4318, 4319, 4320, 4321, 4322, 4323, 4324, 4325, 4326, 4327, 4328, 4329, 4330, 4331, 4332, 4333, 4334, 4335, 4336, 4337, 4338, 4339, 4340, 4341, 4342, 4343, 4344, 4345, 4346, 4347, 4348, 4349, 4350, 4351, 4352, 4353, 4354, 4355, 4356, 4357, 4358, 4359, 4360, 4361, 4362, 4363, 4364, 4365, 4366, 4367, 4368, 4369, 4370, 4371, 4372, 4373, 4374, 4375, 4376, 4377, 4378, 4379, 4380, 4381, 4382, 4383, 4384, 4385, 4386, 4387, 4388, 4389, 4390, 4391, 4392, 4393, 4394, 4395, 4396, 4397) c6(4398, 4399, 4400) c7(T, aaH, aA, nA, Rc, aLl, lE, hb, oW, aT, bNj, K, af, MG, d, Y, dq, kp, aF, ik, DL, dr, rR, kg, aBU, vy, gZ, Lq, aS, ati, lM, wS); #c1(IDUA) c2(XP_011511761) c3(alpha-L-iduronidase isoform X1) c4(302457) c5(302458, 302459, 302460, 302461, 302462, 302463, 302464, 302465, 302466) c6(302467, 302468) c7(T, aaH, aA, nA, Rc, aLl, lE, hb, oW, aT, bNj, K, af, MG, d, Y, dq, kp, aF, iK, DL, dr, rR, kg, aBU, vy, gZ, Lq, aS, ati, lM, wS); #c1(IDUA) c2(XP_011511765) c3(alpha-L-iduronidase isoform X4) c4( ) c5(302469) c6( ) c7(T, aaH, aA, nA, Rc, aLI, lE, hb, oW, aT, bNj, K, af, MG, d, Y, dq, kp, aF, iK, DL, dr, rR, kg, aBU, vy, gZ, Lq, aS, ati, lM, wS); #c1(IER2) c2(NP_004898) c3(immediate early response gene 2 protein) c4(150637) c5(150638, 150639, 150640, 150641, 150642, 150643, 150644) c6( ) c7(T); #c1(IER3) c2(NP_003888) c3(radiation-inducible immediate-early gene IEX-1) c4(141837, 141838, 141839, 141840) c5(141841, 141842, 141843, 141844, 141845, 141846, 141847, 141848, 141849, 141850, 141851, 141852, 141853, 141854, 141855, 141856, 141857, 141858, 141859, 141860, 141861, 141862, 141863, 141864, 141865, 141866, 141867, 141868, 141869, 141870, 141871, 141872, 141873, 141874, 141875, 141876, 141877, 141878, 141879, 141880, 141881, 141882, 141883, 141884, 141885, 141886, 141887, 141888, 141889, 141890, 141891, 141892, 141893, 141894) c6( ) c7(T, aC, dA, fR, aA, cz, fd, V, q, mC, eA, aE, aT, ji, K, lQ, jj, hH, JI, aW, cr, r, eZ, aq, fe, aF, iR, bw, ck, eU, aDs, k, fL, Pk); #c1(IER31P1) c2(NP_057181) c3(immediate early response 3-interacting protein 1 precursor) c4(189326) c5(189327, 189328, 189329, 189330, 189331, 189332) c6( ) c7(mh, ha, aj, bNl, K, bNk, J); #c1(IER5) c2(NP_057629) c3(immediate early response gene 5 protein) c4(191962, 191963) c5(191964, 191965, 191966, 191967, 191968, 191969, 191970, 191971, 191972, 191973, 191974, 191975, 191976, 191977, 191978, 191979) c6( ) c7(bjW, bt, oe); #c1(IFF01) c2(NP_001034759) c3(intermediate filament family orphan 1 isoform 4) c4(32263, 32264) c5(32265, 32266, 32267, 32268, 32269, 32270, 32271) c6( ) c7(t, m); #c1(IFF01) c2(NP_542768) c3(intermediate filament family orphan 1 isoform 2) c4(233029, 233030) c5(233031, 233032, 233033, 233034, 233035, 233036) c6( ) c7(t, m); #c1(IFF01) c2(XP_006719040) c3(intermediate filament family orphan 1 isoform X10) c4(292184) c5(292185, 292186, 292187, 292188, 292189, 292190) c6( ) c7(t, m); #c1(IFF01) c2(XP_006719044) c3(intermediate filament family orphan 1 isoform X13) c4( ) c5(292191, 292192, 292193, 292194, 292195, 292196, 292197, 292198, 292199, 292200, 292201) c6( ) c7(t, m); #c1(IFF02) c2(XP_011538932) c3(intermediate filament family orphan 2 isoform X1) c4(337014) c5(337015, 337016, 337017, 337018, 337019, 337020) c6( ) c7(ac); #c1(IF(16) c2(NP_001193496) c3(gamma-interferon-inducible protein 16 isoform 1) c4(82231) c5(82232, 82233, 82234, 82235, 82236, 82237, 82238) c6( ) c7(T, kh, fR, bcE, ci, V, mC, er, ad, eW, nv, aT, h, lM, ae, aW, ff, eH, eZ, lo, en, eF, t, ik, iT, fL); #c1(IF116) c2(NP_005522) c3(gamma-interferon-inducible protein 16 isoform 2) c4(157069) c5(157070, 157071, 157072, 157073, 157074, 157075, 157076, 157077, 157078, 157079) c6( ) c7(T, kh, fR, bcE, ci, V, mC, er, ad, eW, nv, aT, h, lM, ae, aW, ff, eH, eZ, lo, en, eF, t, ik, iT, fL); #c1(IF116) c2(XP_005245184) c3(gamma-interferon-inducible protein 16 isoform X1) c4(266341, 266342) c5(266343, 266344, 266345, 266346, 266347, 266348) c6( ) c7(T, kh, fR, bcE, ci, V, mC, er, ad, eW, nv, aT, h, lM, ae, aW, ff, eH, eZ, lo, en, eF, t, ik, lT, fL); #c1(IF116) c2(XP_006711353) c3(gamma-interferon-inducible protein 16 isoform X2) c4(285901, 285902, 285903, 285904) c5(285905, 285906, 285907, 285908, 285909, 285910, 285911, 285912, 285913, 285914, 285915, 285916, 285917, 285918, 285919, 285920, 285921, 285922, 285923, 285924, 285925, 285926, 285927, 285928, 285929, 285930, 285931, 285932, 285933, 285934) c6( ) c7(T, kh, fR, bcE, ci, V, mC, er, ad, eW, nv, aT, h, lM, ae, aW, ff, eH, eZ, lo, en, eF, t, ik, iT, fL); #c1(IF(27) c2(NP_001275885) c3(interferon alpha-inducible protein 27, mitochondrial isoform 1) c4(107283, 107284) c5(107285, 107286, 107287, 107288, 107289, 107290, 107291, 107292, 107293, 107294, 107295, 107296) c6( ) c7(T, ea, if, fd, bB, ci, aE, il, Qk, K, eL, bc, xQ, yo, aW, as, cm, bw, GR, eM, nB, dc, go, vA, cj, m, dD, NT, fN, n, fr, qN, jz, cU, h, dL, aDp, ge, aq, zZ, iO, bP, hR, it, B, Dw, fM, aoE, jx, aA, C, q, eA, XX, aT, dz, ae, af, jf, s, iU, Y, r, en, cl, t, iK, aEp, ck, nM, iM, dy, bt, On, hS, hl, Ke, pY, jh, eq, ad, hX, hd, F, ib, ds, fb, d, je, et, ff, cr, apb, aF, bz, bE, aS, EB); #c1(IF127) c2(NP_001275924) c3(interferon alpha-inducible protein 27, mitochondrial isoform 2) c4(107376, 107377, 107378, 107379, 107380) c5(107381, 107382, 107383, 107384, 107385, 107386, 107387, 107388, 107389, 107390, 107391, 107392, 107393, 107394, 107395, 107396, 107397, 107398, 107399, 107400, 107401, 107402, 107403, 107404, 107405, 107406, 107407, 107408, 107409, 107410, 107411, 107412, 107413, 107414) c6( ) c7(T, ea, if, fd, bB, ci, aE, il, Qk, K, eL, bc, xQ, yo, aW, as, cm, bw, GR, eM, nB, dc, go, vA, cj, m, dD, NT, fN, n, fr, qN, jz, cU, h, dL, aDp, ge, aq, zZ, iO, bP, hR, it, B, Dw, fM, aoE, jx, aA, C, q, eA, XX, aT, dz, ae, af, jf, s, iU, Y, r, en, cl, t, ik, aEp, ck, nM, iM, dy, bt, On, hS, hl, Ke, pY, jh, eq, ad, hX, hd, F, ib, ds, fb, d, je, et, ff, cr, apb, aF, bz, bE, aS, EB); #c1(IF130) c2(NP_006323) c3(gamma-interferon-inducible lysosomal thiol reductase preproprotein) c4(164033) c5(164034, 164035, 164036, 164037, 164038, 164039) c6( ) c7(gk, sS, Y, e, al, fJ, bcP, aj, dG, v, ei, bt, J); #c1(IF(35) c2(NP_005524) c3(interferon-induced 35 kDa protein) c4(157080) c5(157081, 157082, 157083, 157084, 157085, 157086) c6( ) c7(ds, t, lo, y, afu); #c1(IF135) c2(XP_005257359) c3(interferon-induced 35 kDa protein isoform X1) c4(275068) c5(275069, 275070, 275071, 275072, 275073, 275074) c6( ) c7(ds, t, lo, y, afu); #c1(IF144) c2(XP_011538818) c3(interferon-induced protein 44 isoform X1) c4(336777, 336778, 336779) c5(336780, 336781, 336782, 336783, 336784, 336785) c6( ) c7(Dw, G, T, F, dA, wu, ds, au, jx, kf, n, h, wm, bX, bu, y, ae, ad, VF); #c1(IF144) c2(XP_011538819) c3(interferon-induced protein 44 isoform X2) c4( ) c5(336786, 336787, 336788, 336789, 336790, 336791) c6( ) c7(Dw, G, T, F, dA, wu, ds, au, jx, kf, n, h, wm, bX, bu, y, ae, ad, VF); #c1(IFIH1) c2(NP_071451) c3(interferon-induced helicase C domain-containing protein 1) c4(211764, 211765) c5(211766, 211767, 211768, 211769, 211770, 211771, 211772, 211773, 211774, 211775) c6( ) c7(bL, bM, T, Ez, bNn, fR, lo, aFc, cz, cL, aj, qj, V, mC, q, J, wD, mP, ds, bW, cU, hD, h, K, hK, A, d, qZ, Y, lb, lC, cr, eZ, dN, aiT, lo, hp, CQ, pm, bNm, lY, aBs, Bv, of, fL, y, B, Ex, aMk); #c1(IFIH1) c2(XP_011509931) c3(interferon-induced helicase C domain-containing protein 1 isoform X2) c4( ) c5(300538, 300539, 300540, 300541, 300542, 300543) c6( ) c7(bL, bM, T, Ez, bNn, fR, lo, aFc, cz, cL, aj, qj, V, mC, q, J, wD, mP, ds, bW, cU, hD, h, K, hK, A, d, qZ, Y, lb, lC, cr, eZ, dN, aiT, lo, hp, CQ, pm, bNm, lY, aBs, Bv, of, fL, y, B, Ex, aMk); #c1(IFIT1) c2(NP_001257858) c3(interferon-induced protein with tetratricopeptide repeats 1 isoform 3) c4( ) c5(95671, 95672, 95673, 95674, 95675) c6( ) c7(dP, T, Bv, fR, ds, aw, Be, eZ, lo, Xh, as, t); #c1(IFIT1) c2(NP_001539) c3(interferon-induced protein with tetratricopeptide repeats 1 isoform 1) c4(118703, 118704) c5(118705, 118706, 118707, 118708, 118709, 118710, 118711) c6( ) c7(dP, T, Bv, fR, ds, aw, Be, eZ, lo, Xh, as, t); #c1(IFIT2) c2(NP_001538) c3(interferon-induced protein with tetratricopeptide repeats 2) c4(118696) c5(118697, 118698, 118699, 118700, 118701, 118702) c6( ) c7(fR, ds, Y, ff, aiM, cu, lo, ci, en, q); #c1(IFIT3) c2(NP_001276687) c3(interferon-induced protein with tetratricopeptide repeats 3 isoform b) c4( ) c5(108269, 108270, 108271, 108272, 108273, 108274) c6( ) c7(ck, hG, ds, U, aT, eZ, EY, f, nJ); #c1(IFIT3) c2(NP_001540) c3(interferon-induced protein with tetratricopeptide repeats 3 isoform a) c4(118712, 118713) c5(118714, 118715, 118716, 118717, 118718, 118719, 118720, 118721, 118722, 118723, 118724) c6( ) c7(ck, hG, ds, U, aT, eZ, EY, f, nJ); #c1(IFIT5) c2(NP_036552) c3(interferon-induced protein with tetratricopeptide repeats 5) c4(174358, 174359) c5(174360, 174361, 174362, 174363, 174364, 174365, 174366, 174367, 174368, 174369, 174370, 174371) c6( ) c7(ds); #c1(IFITM1) c2(NP_003632) c3(interferon-induced transmembrane protein 1) c4(139724, 139725) c5(139726, 139727, 139728, 139729, 139730, 139731, 139732, 139733, 139734, 139735, 139736, 139737, 139738, 139739, 139740, 139741, 139742, 139743, 139744, 139745) c6( ) c7(T, ht, jx, aA, r, Th, aF, g, t, q); #c1(IFITM2) c2(NP_006426) c3(interferon-induced transmembrane protein 2) c4(165119, 165120) c5(165121, 165122, 165123, 165124, 165125, 165126, 165127, 165128, 165129, 165130, 165131, 165132) c6( ) c7(bE, t); #c1(IFITM3) c2(NP_066362) c3(interferon-induced transmembrane protein 3) c4(207211, 207212) c5(207213, 207214, 207215, 207216, 207217, 207218, 207219, 207220, 207221, 207222, 207223, 207224) c6( ) c7(T, s, bE, ds, Y, bB, lo, uA, fD, iV, X, afu); #c1(IFITM5) c2(NP_001020466) c3(interferon-induced transmembrane protein 5) c4(28814) c5(28815, 28816, 28817, 28818, 28819, 28820) c6( ) c7(zp, bNo, zg, bNp); #c1(IFNA1) c2(NP_076918) c3(interferon alpha-1113 precursor) c4(214226) c5(214227, 214228, 214229, 214230, 214231, 214232, 214233, 214234) c6(214235, 214236) c7(ID, bNu, T, os, bM, aC, fR, ea, al, fd, bB, gf, qj, ci, bh, Jx, aY, x, AW, xy, aE, bW, ajv, dV, Ds, K, hH, pk, eL, aa, ly, aW, bNq, az, hl, zD, asD, aiT, uY, fc, aoD, g, eF, ir, cm, iP, bNr, y, eM, nB, xD, dc, iA, iT, oy, aiM, cj, k, m, gn, hh, NK, Ez, dD, aik, gp, Or, yG, bzb, fN, n, aj, oY, fr, ct, apt, KD, qW, ai, eh, eW, BI, ft, zw, pe, rC, gd, cU, h, hn, dL, DM, cY, afu, Nr, mR, A, lb, aJG, fQ, aq, po, kp, yq, CQ, pm, Ca, kk, bNw, lx, Wt, NL, hk, hR, uz, pg, eo, fL, bkE, Pr, B, it, Dw, aoT, fM, dA, eR, jx, aA, v, j, ON, bX, V, q, J, eA, zn, eJ, EB, XX, bNv, lg, Vr, jZ, rS, aCV, aT, cy, dz, qG, ae, VC, hM, Uf, iU, nD, Y, ev, r, lo, aDm, en, t, f, ik, awi, bNt, pb, ck, cT, sS, iM, Dy, iQ, AH, bdP, wR, Da, dy, yH, bt, ao, bNx, vi, hS, Kd, ra, BD, bxK, uG, bNs, jh, oZ, bS, eq, jE, Fb, wV, bkM, hF, ase, mC, ad, hX, qC, av, hd, F, ib, Rq, afm, Ot, ds, lC, oW, Os, ji, nJ, uQ, atJ, d, bkf, je, ff, wc, gC, eH, cr, kD, eZ, aF, hp, BG, avG, Z, iR, iV, bz, Nq, es, gk, Bv, pc, bE, z, ag, akC, bNy, dX, XO, rH, cP, aEN, hE, xg, Ex, pC); #c1(IFNA10) c2(NP_002162) c3(interferon alpha-10 precursor) c4(126096) c5(126097, 126098, 126099, 126100, 126101, 126102, 126103, 126104) c6(126105, 126106) c7(hk, Y, vB, vi); #c1(IFNA13) c2(NP_008831) c3(interferon alpha-1113 precursor) c4(168900) c5(168901, 168902, 168903, 168904, 168905, 168906, 168907, 168908) c6(168909, 168910) c7(ID, bNu, T, os, bM, ea, al, fd, bB, gf, qj, ci, bh, Jx, aY, x, AW, xy, aE, bW, ajv, dV, Ds, K, hH, pk, eL, aa, ly, aW, bNq, az, hl, zD, asD, aiT, uY, fc, aoD, g, eF, ir, cm, iP, bNr, y, eM, nB, xD, dc, iA, iT, oy, aiM, cj, Dt, k, m, gn, hh, NK, Ez, dD, aik, gp, Or, yG, bzb, n, aj, oY, fr, ct, apt, kQ, qW, ai, eh, eW, BI, ft, zw, pe, rC, gd, cU, h, hn, dL, OM, cY, afu, Nr, mR, A, lb, aq, po, kp, yq, CQ, pm, Ca, kk, lx, Wt, NL, hR, uz, pg, eo, fL, bkE, Pr, B, it, Dw, aoT, fM, dA, eR, jx, aA, v, j, ON, bX, V, q, J, eA, zn, eJ, EB, XX, bNv, lg, Vr, jZ, rS, aCV, aT, cy, dz, qG, ae, VC, hM, Uf, iU, nO, Y, ev, r, lo, aDm, en, t, f, ik, awi, bNt, pb, ck, cT, sS, iM, Dy, AH, bdP, wR, Da, dy, yH, bt, ao, vi, hS, Kd, BD, bxK, uG, bNs, jh, oZ, bS, eq, jE, Fb, wV, bkM, hF, ase, mC, ad, hX, qC, av, hd, F, ib, Rq, afm, Ot, ds, lC, oW, Os, ji, nJ, uQ, atJ, d, bkf, je, ff, wc, gC, eH, cr, kD, eZ, aF, hp, BG, avG, Z, iR, iV, bz, Nq, es, gk, Bv, pc, bE, z, ag, akC, bNy, dX, XO, rH, cP, aEN, hE, xg, Ex, pC); #c1(IFNA14) c2(NP_002163) c3(interferon alpha-14 precursor) c4(126107) c5(126108, 126109, 126110, 126111, 126112, 126113, 126114, 126115) c6(126116, 126117) c7(ck, Dw, bM, hk, Y, Ds, vi); #c1(IFNA16) c2(NP_002164) c3(interferon alpha-16 precursor) c4(126118) c5(126119, 126120, 126121, 126122, 126123, 126124, 126125, 126126) c6(126127, 126128) c7(hk, Y); #c1(IFNA17) c2(NP_067091) c3(interferon alpha-17 precursor) c4(208960) c5(208961, 208962, 208963, 208964, 208965, 208966, 208967, 208968) c6(208969, 208970) c7(T, if, jx, bkM, qN, V, av, Rq, vB, eL, uU, d, iU, nO, Y, ff, eZ, lo, en, g, t, hk, hR, m, bkE, it, cn, hS); #c1(IFNA2) c2(NP_000596) c3(interferon alpha-2 precursor) c4(13535) c5(13536, 13537, 13538, 13539, 13540, 13541, 13542, 13543, 13544, 13545, 13546, 13547, 13548, 13549, 13550, 13551, 13552, 13553, 13554, 13555, 13556, 13557, 13558, 13559, 13560, 13561, 13562, 13563, 13564, 13565, 13566, 13567, 13568, 13569, 13570, 13571, 13572, 13573, 13574, 13575, 13576, 13577, 13578, 13579, 13580, 13581, 13582, 13583, 13584, 13585, 13586, 13587, 13588, 13589, 13590, 13591, 13592, 13593, 13594, 13595, 13596, 13597, 13598, 13599, 13600, 13601, 13602, 13603, 13604, 13605, 13606, 13607, 13608, 13609, 13610, 13611, 13612, 13613, 13614, 13615, 13616, 13617, 13618, 13619, 13620, 13621, 13622, 13623, 13624, 13625, 13626, 13627, 13628, 13629, 13630) c6(13631, 13632, 13633) c7(bM, T, aC, boZ, al, hE, fd, aeu, ip, dV, Ds, aoV, eL, HA, hl, fc, g, eE, cm, bw, bNr, nB, dc, oy, m, Vj, gp, aw, n, fr, ct, qN, cd, qg, aZv, h, eV, dL, DU, he, A, bNA, bep, fQ, pm, hq, hk, uw, hR, E, pg, bkE, it, B, Dw, jx, eX, C, XX, Vr, aCV, ae, VC, iU, nO, Y, lo, en, t, vk, bNB, BU, rR, iM, hc, gX, fP, hS, vi, gV, uq, id, iW, bNz, bkM, ah, jE, hF, ad, hX, qC, av, F, Rq, qh, oW, hD, lQ, uQ, d, lR, je, hG, et, ff, eZ, kD, S, avG, gk, pc, akC, lC, EB); #c1(IFNA21) c2(NP_002166) c3(interferon alpha-21 precursor) c4(126129) c5(126130, 126131, 126132, 126133, 126134, 126135, 126136, 126137) c6(126138, 126139) c7(hk, Y, jx, cF); #c1(IFNA4) c2(NP_066546) c3(interferon alpha-4 precursor) c4(207333) c5(207334, 207335, 207336, 207337, 207338, 207339, 207340, 207341) c6(207342, 207343) c7(F, fR, vi); #c1(IFNA5) c2(NP_002160) c3(interferon alpha-5 precursor) c4(126074) c5(126075, 126076, 126077, 126078, 126079, 126080, (26081, 126082) c6(126083, 126084) c7(Dw, aC, lg, hk, Y, jx, eZ, aq, jE, aDp, B, vi); #c1(IFNA6) c2(NP_066282) c3(interferon alpha-6 precursor) c4(206963) c5(206964, 206965, 206966, 206967, 206968, 206969, 206970, 206971) c6(206972, 206973) c7(bM, Dw, F, hk, ds, Y, jx, Ds, vi); #c1(IFNA7) c2(NP_066401) c3(interferon alpha-7 precursor) c4(207308) c5(207309, 207310, 207311, 207312, 207313, 207314, 207315, 207316) c6(207317, 207318) c7(vi); #c1(IFNA8) c2(NP_002161) c3(interferon alpha-8 precursor) c4(126085) c5(126086, 126087, 126088, 126089, 126090, 126091, 126092, 126093) c6(126094, (26095) c7(bM, Dw, hk, Y, oW, Ds, vi); #c1(IFNAR1) c2(NP_000620) c3(interferon alpha/beta receptor 1 precursor) c4(14000, 14001, 14002) c5(14003, 14004, 14005, 14006, 14007, 14008, 14009, 14010, 14011, 14012, 14013, 14014, 14015, 14016, 14017, 14018, 14019, 14020) c6( ) c7(ID, gV, bM, Dw, aC, fR, al, jx, aA, bD, fd, amk, Ja, bS, ct, jE, pl, V, eh, mC, q, ai, hX, F, aE, adG, ds, dV, Os, aAn, adD, Ds, dL, DU, K, h, eL, ly, hG, adF, eZ, r, fe, aq, lo, aF, ir, Z, Rp, f, cm, pb, bNC, lx, hq, iT, AH, dX, dy, fL, bdR, hh, B, bND); #c1(IFNAR2) c2(NP_997467) c3(interferon alpha/beta receptor 2 isoform b) c4(265549) c5(265550, 265551, 265552, 265553, 265554, 265555, 265556, 265557, 265558, 265559, 265560) c6( ) c7(gV, bM, dD, Dw, dA, fR, aC, al, bNE, jx, fd, pl, eq, jE, fr, V, hX, F, aE, Ds, dL, DU, hH, pk, CM, lo, CQ, f, pb, RB, iT, at, Da, Dt, eo, B); #c1(IFNAR2) c2(NP_997468) c3(interferon alpha/beta receptor 2 isoform a) c4(265561) c5(265562, 265563, 265564, 265565, 265566, 265567, 265568, 265569, 265570, 265571) c6( ) c7(gV, bM, dD, Dw, dA, fR, aC, al, bNE, jx, fd, pl, eq, jE, fr, V, hX, F, aE, Ds, dL, DU, hH, pk, CM, lo, CQ, f, pb, RB, iT, at, Da, Dt, eo, B); #c1(IFNB1) c2(NP_002167) c3(interferon beta precursor) c4(126140) c5(126141, 126142, 126143, 126144, 126145, 126146, 126147, 126148, 126149, 126150, 126151, 126152, 126153, 126154, 126155, 126156, 126157, 126158, 126159, 126160, 126161, 126162, 126163, 126164, 126165, 126166, 126167, 126168, 126169, 126170, 126171, 126172, 126173, 126174, 126175, 126176, 126177, 126178, 126179, 126180, 126181, 126182, 126183, 126184, 126185, 126186, 126187, 126188, 126189, 126190, 126191, 126192, 126193, 126194, 126195, 126196, 126197, 126198, 126199, 126200, 126201, 126202, 126203, 126204, 126205, 126206, 126207, 126208, 126209, 126210, 126211, 126212, 126213, 126214, 126215, 126216, 126217, 126218, 126219, 126220, 126221, 126222, 126223, 126224, 126225, 126226, 126227, 126228, 126229, 126230, 126231, 126232, 126233, 126234, 126235) c6(126236, 126237, 126238) c7(oO, bM, T, aC, aEn, fR, if, fd, bB, ci, mF, aJO, x, AW, M, dV, iv, K, cp, akV, bkr, iE, aW, asD, aiT, La, as, cm, y, eM, bNF, aiM, mx, zv, k, m, gn, fN, qy, fr, qN, aDJ, eW, Mo, cU, h, hn, pi, cY, afu, nP, A, aiB, oo, lb, CQ, kk, RB, bP, dB, hk, eD, hL, eo, it, B, dA, jx, aA, v, V, q, eA, lg, hb, aT, aAn, yu, cy, ae, af, bA, jf, azU, Uf, s, nO, iU, oV, Y, Pg, r, lo, en, cl, t, ck, wg, ac, bxj, Da, bt, vi, Ke, iW, jh, wV, pl, jE, fu, hF, ad, hX, qC, F, ib, aaU, xl, ds, uQ, d, ff, wc, rU, eZ, aF, bz, Bv, bE, agl, hE, Ex); #c1(IFNE) c2(NP_795372) c3(interferon epsilon precursor) c4(252444) c5(252445, 252446, 252447, 252448, 252449, 252450, 252451, 252452) c6(252453, 252454) c7(oO, ac, hk, Y, e, j, on, fq); #c1(IFNG) c2(NP_000610) c3(interferon gamma precursor) c4(13736) c5(13737, 13738, 13739, 13740, 13741, 13742) c6( ) c7(T, os, ea, aHH, bO, fd, ci, aY, bv, azY, xy, aE, dV, Ds, Bi, hu, hH, LG, azh, cp, VF, aa, az, ba, fT, iA, Jr, dc, cj, Dt, k, eg, bhp, aYZ, ht, aj, qW, cd, br, zo, baA, auj, aZv, lG, aNj, ey, bEg, pl, mR, Cw, sp, aRL, fQ, fe, bNY, CQ, adz, iO, dB, bOj, bjx, ajd, abW, aZy, DD, aA, cz, bX, wy, q, zn, bNX, bOc, bNQ, aWc, aCX, bOk, kf, aLM, j, rz, DX, bf, fO, bg, lo, pb, Cv, ov, cT, pa, OW, iM, iQ, ig, Nk, aXt, tz, bNH, bt, ao, gV, aCA, iW, jh, oZ, dG, ad, afb, aoH, hX, wD, Dq, oW, bj, lQ, qZ, ff, aLI, aZw, Mz, bNZ, Sm, iV, bz, aZz, DF, aDH, Om, anY, aDB, oQ, lf, ra, ne, bsJ, oO, bM, xS, qn, qo, bkF, if, e, bOa, bB, cL, bh, sD, Tz, x, fS, azm, bc, akX, eG, hl, bOg, VE, as, aFU, BE, eF, aiv, cm, bw, xD, bmt, Dv, ym, hh, abh, avY, aiu, dD, JZ, akc, yG, n, oY, Mo, b, Xe, Bj, aDa, eV, hn, pi, A, fJ, lb, QP, po, yq, amo, cA, Pa, at, ut, eo, fL, bNT, B, NQ, dA, eX, v, V, aGT, rS, ld, aDt, ae, blm, jf, hM, ev, aFK, afq, acB, bu, t, nC, ck, auT, oX, yH, Gg, bOo, vi, hl, MH, df, wV, eq, qi, bNM, bNN, afm, ds, LA, hD, rN, JK, nJ, Ng, d, je, azX, wc, aFt, CF, dN, aCK, Z, aGj, bOf, dr, aDN, D, gk, Ny, agl, y, kS, hv, pC, aC, fR, al, CH, axk, bNU, qj, lR, Qp, wa, bDI, AW, bOm, bOn, xE, bW, K, wt, DY, dF, eL, bNG, mH, blP, bOh, axu, asD, mQ, aGQ, aB, g, ve, uC, iP, wT, aik, yl, xK, aDs, aWZ, AM, aer, uo, dW, m, Ro, NK, aFQ, sQ, sw, aw, fN, BH, BF, apt, aBl, pM, aCQ, eh, ft, bKk, kH, cU, aDp, gi, arp, cM, fq, Zj, ar, kT, acC, aZL, mg, hs, gg, bNS, bOe, bNV, Pr, it, L, bOi, fM, fG, jx, j, aYW, pd, eA, J, o, er, XX, lg, hb, bNO, jZ, qG, bq, bNR, xR, bsb, Y, Pg, r, en, zM, aER, bN, QG, aQd, c, hc, wR, bxj, rK, dy, fD, gx, id, bxK, em, bs, auq, bS, pl, EA, oH, hF, ase, dP, bOd, oj, vB, aNp, bNL, ji, NM, asn, lU, eH, cr, hp, Fq, KM, bNW, Nq, Lj, aFb, eB, aEs, aCl, ei, hE, st, EB, bol, aFg, aAx, bvq, mF, fw, aR, iX, Ou, aWf, H, bhm, bat, hm, DA, akV, aW, HA, yd, eM, iT, uJ, zv, gn, jM, aSs, aFc, Bm, fr, ct, qN, KO, alq, aPb, ai, eW, BI, cc, ju, zf, axE, jy, h, dL, Xh, afu, Nr, oo, Cu, bJl, bmu, aCE, aq, kp, mo, Ca, gG, kk, Wt, lX, bm, hR, uz, oh, aDE, aMk, bL, Dw, lk, eR, dk, rf, bNl, C, ze, afv, ady, ga, bob, mP, HC, aT, aSX, an, af, iU, oV, aYp, aDm, Ph, ajF, f, sS, Zg, vZ, lV, bNJ, fP, aYx, mf, aDj, hS, Kd, Ke, ZI, qb, aD, vr, jE, pz, bNK, mC, KL, LH, qC, av, F, ib, oz, Os, jo, yF, uQ, yx, eZ, yy, xv, aF, WJ, LB, es, bhO, eU, zr, atM, bE, z, aS, be, bNP, fh, X); #c1(IFNGR1) c2(NP_000407) c3(interferon gamma receptor 1 precursor) c4(8611, 8612, 8613, 8614) c5(8615, 8616, 8617, 8618, 8619, 8620, 8621, 8622, 8623, 8624, 8625, 8626, 8627, 8628, 8629, 8630, 8631, 8632) c6( ) c7(bM, sY, bDi, T, bOw, FR, fM, dD, Dw, eq, axd, Ju, ht, al, aA, eb, jx, NT, wV, bNV, jE, bOq, bOt, ci, j, EA, eh, mC, st, Qp, hX, dP, cF, F, fS, bOr, dV, aT, h, aDa, cy, dL, bOp, ey, hu, bOu, af, pl, aa, uQ, li, qZ, iU, aW, sp, bg, eZ, kt, fe, aF, aCK, eE, bOv, uC, adz, ba, kT, fT, ig, z, aD, bOs, cj, hs, uz, at, fL, eg, y, bhp, ao, vi); #c1(IFNGR1) c2(XP_011534096) c3(interferon gamma receptor 1 isoform X1) c4(330334, 330335) c5(330336, 330337, 330338, 330339, 330340, 330341, 330342, 330343, 330344, 330345, 330346, 330347, 330348, 330349, 330350, 330351, 330352, 330353) c6( ) c7(bM, sY, bDi, T, bOw, FR, fM, dD, Dw, eq, axd, Ju, ht, al, aA, eb, jx, NT, wV, bNV, jE, bOq, bOt, ci, j, EA, eh, mC, st, Qp, hX, dP, cF, F, fS, bOr, dV, aT, h, aDa, cy, dL, bOp, ey, hu, bOu, af, pl, aa, uQ, li, qZ, iU, aW, sp, bg, eZ, kt, fe, aF, aCK, eE, bOv, uC, adz, ba, kT, fT, ig, z, aD, bOs, cj, hs, uz, at, fL, eg, y, bhp, ao, vi); #c1(IFNGR2) c2(NP_005525) c3(interferon gamma receptor 2 precursor) c4(157087) c5(157088, 157089, 157090, 157091, 157092, 157093) c6( ) c7(bM, sY, dD, Dw, fR, wu, iW, al, NT, aA, wV, j, jE, bX, eq, V, hX, F, aT, Ds, dL, bOp, wm, uQ, aa, A, qZ, iU, aW, sp, ff, eZ, af, en, uC, kT, ig, z, at, fL, zA, st); #c1(IFNK) c2(NP_064509) c3(interferon kappa precursor) c4(202694) c5(202695, 202696, 202697, 202698, 202699, 202700, 202701, 202702) c6(202703, 202704) c7(c, hk, Y, eZ, qN, y, cd, afu); #c1(IFNL1) c2(NP_742152) c3(interferon lambda-1 precursor) c4(248217) c5(248218, 248219, 248220, 248221, 248222, 248223) c6( ) c7(bM, eW, F, mR, fR, rw, aZv, jx, cr, aq, lo, hE, aYF, NM, B, y, hX); #c1(IFNL2) c2(XP_011525058) c3(interferon lambda-2 isoform X1) c4(319515) c5(319516, 319517, 319518, 319519, 319520, 319521) c6( ) c7(bM, F, id, fR, jx, hl, dL, eL); #c1(IFNL3) c2(NP_742151) c3(interferon lambda-3 precursor) c4( ) c5(248212, 248213, 248214, 248215, 248216) c6( ) c7(bL, Kd, bM, Ke, al, jx, n, aj, v, jE, V, eW, J, aoH, hX, F, no, aZv, oW, h, rB, XI, je, lR, fc, hl, Mz, aiT, dq, lo, byM, eE, cM, ik, gk, cT, BU, W, nb, M, dc, NL, Gs, bcl, cP, hE, B, EB, vi); #c1(IFNL3) c2(XP_011525059) c3(interferon lambda-3 isoform X1) c4(319522) c5(319523) c6( ) c7(bL, Kd, bM, Ke, al, jx, n, aj, v, jE, V, eW, J, aoH, hX, F, no, aZv, oW, h, rB, XI, je, lR, fc, hl, Mz, aiT, dq, lo, byM, eE, cM, ik, gk, cT, BU, W, nb, iM, dc, NL, Gs, bcl, cP, hE, B, EB, vi); #c1(IFNW1) c2(NP_002168) c3(interferon omega-1 precursor) c4(126239) c5(126240, 126241, 126242, 126243, 126244, 126245) c6( ) c7(hk, Y, A, jx, cF); #c1(IFRD1) c2(NP_001007246) c3(interferon-related developmental regulator 1 isoform 1) c4(22619, 22620, 22621) c5(22622, 22623, 22624, 22625, 22626, 22627, 22628, 22629, 22630, 22631, 22632, 22633, 22634, 22635, 22636, 22637, 22638, 22639, 22640, 22641, 22642) c6( ) c7(eM, j, ci, yb, cd, ao, aa); #c1(IFRD1) c2(NP_001184008) c3(interferon-related developmental regulator 1 isoform 2) c4( ) c5(75246, 75247, 75248, 75249, 75250, 75251, 75252, 75253, 75254, 75255, 75256) c6( ) c7(eM, j, ci, yb, cd, ao, aa); #c1(IFRD1) c2(XP_011514444) c3(interferon-related developmental regulator 1 isoform X1) c4(307134, 307135, 307136) c5(307137, 307138, 307139, 307140, 307141, 307142, 307143, 307144, 307145, 307146) c6( ) c7(eM, j, ci, yb, cd, ao, aa); #c1(IFT122) c2(NP_001267475) c3(intraflagellar transport protein 122 homolog isoform 7) c4( ) c5(99594, 99595, 99596, 99597, 99598) c6( ) c7(arv, ul, aV, Pf, bhD, adv, bwv, box, dy, zV, bee, bly, dr); #c1(IFT122) c2(NP_060732) c3(intraflagellar transport protein 122 homolog isoform 3) c4(197498, 197499, 197500) c5(197501, 197502, 197503, 197504, 197505, 197506, 197507, 197508, 197509, 197510, 197511, 197512, 197513) c6( ) c7(arv, ul, aV, Pf, bhD, adv, bvv, box, dy, zV, bee, bly, dr); #c1(IFT122) c2(NP_443711) c3(intraflagellar transport protein 122 homolog isoform 1) c4(229943) c5(229944, 229945, 229946, 229947, 229948, 229949) c6( ) c7(arv, ul, aV, Pf, bhD, adv, bvv, box, dy, zV, bee, bly, dr); #c1(IFT122) c2(NP_443715) c3(intraflagellar transport protein 122 homolog isoform 2) c4(229969, 229970) c5(229971, 229972, 229973, 229974, 229975, 229976, 229977, 229978, 229979, 229980, 229981, 229982) c6( ) c7(arv, ul, aV, Pf, bhD, adv, bvv, box, dy, zV, bee, bly, dr); #c1(IFT122) c2(NP_443716) c3(intraflagellar transport protein 122 homolog isoform 4) c4(229983, 229984, 229985) c5(229986, 229987, 229988, 229989, 229990, 229991, 229992, 229993, 229994, 229995, 229996, 229997, 229998, 229999, 230000, 230001, 230002, 230003) c6( ) c7(arv, ul, aV, Pf, bhD, adv, bvv, box, dy, zV, bee, bly, dr); #c1(IFT140) c2(NP_055529) c3(intraflagellar transport protein 140 homolog) c4(181788, 181789, 181790) c5(181791, 181792, 181793, 181794, 181795, 181796, 181797, 181798, 181799, 181800, 181801, 181802, 181803, 181804, 181805) c6( ) c7(EP, bjb, nx, dy); #c1(IFT140) c2(XP_005255783) c3(intraflagellar transport protein 140 homolog isoform X9) c4( ) c5(273873, 273874, 273875, 273876, 273877, 273878) c6( ) c7(EP, bjb, nx, dy); #c1(IFT140) c2(XP_006721055) c3(intraflagellar transport protein 140 homolog isoform X10) c4( ) c5(293830) c6( ) c7(EP, bjb, nx, dy); #c1(IFT172) c2(NP_056477) c3(intraflagellar transport protein 172 homolog) c4(187694) c5(187695, 187696, 187697, 187698, 187699, 187700, 187701, 187702, 187703, 187704, 187705, 187706, 187707) c6( ) c7(iM, je, bjb, kH, sx, BM, bOz, bOA); #c1(IFT172) c2(XP_005264311) c3(intraflagellar transport protein 172 homolog isoform X2) c4(278803) c5( ) c6( ) c7(iM, je, bjb, kH, sx, BM, bOz, bOA); #c1(IFT27) c2(NP_001171172) c3(intraflagellar transport protein 27 homolog isoform 1) c4(69975, 69976, 69977) c5(69978, 69979, 69980, 69981, 69982, 69983, 69984, 69985, 69986, 69987, 69988, 69989, 69990, 69991) c6( ) c7(bK, Tp); #c1(IFT27) c2(NP_006851) c3(intraflagellar transport protein 27 homolog isoform 2) c4(168623, 168624, 168625, 168626) c5(168627, 168628, 168629, 168630, 168631, 168632, 168633, 168634, 168635, 168636, 168637, 168638, 168639, 168640, 168641, 168642, 168643, 168644, 168645, 168646, 168647, 168648, 168649, 168650, 168651, 168652, 168653, 168654) c6( ) c7(bK, Tp); #c1(IFT43) c2(NP_001096034) c3(intraflagellar transport protein 43 homolog isoform 2) c4(40982) c5(40983, 40984, 40985, 40986, 40987, 40988) c6( ) c7(bOB, oe, bOx); #c1(IFT43) c2(NP_001242924) c3(intraflagellar transport protein 43 homolog isoform 3) c4(89276) c5(89277, 89278, 89279, 89280, 89281, 89282) c6( ) c7(bOB, oe, bOx); #c1(IFT43) c2(NP_443105) c3(intraflagellar transport protein 43 homolog isoform 1) c4(229446) c5(229447, 229448, 229449, 229450, 229451, 229452) c6( ) c7(bOB, oe, bOx); #c1(IFT57) c2(NP_060480) c3(intraflagellar transport protein 57 homolog) c4(196159) c5(196160, 196161, 196162, 196163, 196164, 196165) c6( ) c7(rR, d); #c1(IFT74) c2(NP_001092693) c3(intraflagellar transport protein 74 homolog isoform a) c4(39557, 39558) c5(39559, 39560, 39561, 39562, 39563, 39564, 39565, 39566, 39567, 39568, 39569, 39570, 39571, 39572) c6( ) c7(aw, c); #c1(IFT74) c2(NP_001092694) c3(intraflagellar transport protein 74 homolog isoform b) c4(39573, 39574) c5(39575, 39576, 39577, 39578, 39579, 39580, 39581, 39582, 39583, 39584, 39585, 39586, 39587, 39588, 39589, 39590, 39591, 39592) c6( ) c7(aw, c); #c1(IFT74) c2(XP_011516337) c3(intraflagellar transport protein 74 homolog isoform X1) c4(309567) c5(309568, 309569, 309570, 309571, 309572, 309573) c6( ) c7(aw, c); #c1(IFT80) c2(NP_001177171) c3(intraflagellar transport protein 80 homolog isoform b) c4(72492) c5(72493, 72494, 72495, 72496, 72497, 72498, 72499, 72500) c6( ) c7(EP, bja, nw, zG, bOC); #c1(IFT80) c2(NP_065851) c3(intraflagellar transport protein 80 homolog isoform a) c4(205875, 205876, 205877, 205878) c5(205879, 205880, 205881, 205882, 205883, 205884, 205885, 205886, 205887, 205888, 205889, 205890, 205891, 205892, 205893, 205894, 205895, 205896, 205897, 205898, 205899, 205900, 205901, 205902, 205903, 205904) c6( ) c7(EP, bja, nw, zG, bOC); #c1(IFT88) c2(XP_005266603) c3(intraflagellar transport protein 88 homolog isoform X3) c4( ) c5(280253) c6( ) c7(asp, wB, aq, aS, ct, zG, Nj, anN, B); #c1(IFT88) c2(XP_005266604) c3(intraflagellar transport protein 88 homolog isoform X5) c4( ) c5(280254) c6( ) c7(asp, wB, aq, aS, ct, zG, Nj, anN, B); #c1(IFT88) c2(XP_005266606) c3(intraflagellar transport protein 88 homolog isoform X7) c4(280255) c5(280256, 280257, 280258, 280259, 280260, 280261) c6( ) c7(asp, wB, aq, aS, ct, zG, Nj, anN, B); #c1(IFT88) c2(XP_005266607) c3(intraflagellar transport protein 88 homolog isoform X8) c4(280262) c5(280263, 280264) c6( ) c7(asp, wB, aq, aS, ct, zG, Nj, anN, B); #c1(IFT88) c2(XP_005266609) c3(intraflagellar transport protein 88 homolog isoform X10) c4(280265) c5(280266, 280267, 280268, 280269, 280270, 280271) c6( ) c7(asp, wB, aq, aS, ct, zG, Nj, anN, B); #c1(IFT88) c2(XP_011533548) c3(intraflagellar transport protein 88 homolog isoform X13) c4(329659) c5(329660, 329661, 329662, 329663, 329664, 329665, 329666, 329667, 329668) c6( ) c7(asp, wB, aq, aS, ct, zG, Nj, anN, B); #c1(IGBP1) c2(NP_001542) c3(immunoglobulin-binding protein 1) c4(118725) c5(118726, 118727, 118728, 118729, 118730, 118731, 118732, 118733, 118734, 118735) c6( ) c7(wS, cV, lr, fd, jh, fr, HS, uE, bFj, aY, V, q, eW, av, Mo, no, aT, ta, ji, aSS, aAe, pi, Qe, bOD, A, Gv, aml, Y, bfV, r, fe, t, bz, ZL, BU, qe, zr, cj, Da, eo, m, ER, aSQ, y, bt, aNX, B, aNW, XL); #c1(IGDCC3) c2(NP_004875) c3(immunoglobulin superfamily DCC subclass member 3 precursor) c4(150466) c5(150467, 150468, 150469, 150470, 150471, 150472, 150473) c6( ) c7(aW, n, aT); #c1(IGDCC3) c2(XP_011520543) c3(immunoglobulin superfamily DCC subclass member 3 isoform X1) c4(315469) c5(315470, 315471, 315472, 315473, 315474, 315475) c6( ) c7(aW, n, aT); #c1(IGF1) c2(NP_000609) c3(insulin-like growth factor I isoform 4 preproprotein) c4(13724) c5(13725, 13726, 13727, 13728, 13729, 13730, 13731, 13732, 13733) c6(13734, 13735) c7(T, ea, fd, ci, aY, GK, bv, aE, fm, dV, hu, avM, cp, li, yo, ZH, Ls, zE, iA, nB, cj, bOJ, FG, ht, cd, aj, cd, Ak, ey, bl, fQ, fe, pm, AK, ok, dB, eD, bOE, Be, E, bOF, AN, jv, aG, aA, cz, bX, bol, avO, q, kf, cy, rv, eu, bA, iF, nO, fO, cl, pb, bOG, rR, acW, ac, tW, mG, hr, cG, aDR, aps, bt, rA, bMo, iW, jh, KO, dG, ad, hX, yt, vJ, lQ, ahk, BO, cW, PE, ff, Of, dq, bz, OG, ag, aed, iu, if, e, bB, brq, ip, bc, Ur, hl, as, eF, cm, aiv, lt, vA, mx, vy, tV, bBT, Ht, dD, yG, n, nA, aje, cq, pe, eV, avP, A, RT, Rj, lb, hQ, yq, bP, PI, hL, at, ut, B, tq, vQ, AS, dA, eX, fF, GC, v, Qo, V, eJ, Kx, UE, hW, ae, jf, ev, asy, bbs, aom, bu, t, aoy, aff, zJ, kP, wO, gX, zu, xb, hl, acl, RH, OR, zF, eq, hd, aop, LA, hD, vc, Dj, d, Z, HI, dr, D, gk, ll, gZ, Kj, y, ng, aC, FR, azf, yS, atj, al, bW, pX, M, K, eL, adT, BBQ, ago, apn, LZ, g, uC, iP, AM, U, uo, m, bBJ, sQ, OI, aw, NT, fg, aCQ, zP, JW, ft, uV, dx, CN, Og, W, anZ, wk, bo, it, ail, fG, qQ, aee, hz, j, sl, eA, J, XX, MR, lg, bBN, bq, kl, Y, LO, r, en, wA, iK, bN, Qi, Dy, c, kB, hc, dy, fD, gT, gx, sY, kh, id, bxK, aZ, bS, iJ, dP, fU, aet, tt, bKm, Yi, uX, lR, aus, xs, eH, cr, Fq, Bu, qa, pu, nb, AR, sG, bC, ei, vt, xg, G, pf, acz, fV, amj, bBb, mF, dY, il, iX, Ou, zQ, aW, hJ, eM, iT, aYS, gn, pw, ez, fr, ct, qN, rc, ke, cc, hw, rl, zf, h, jy, dL, Xh, Um, Ck, Dg, aq, kk, op, bJ, Qy, hR, arg, bL, eR, yk, hy, asA, uT, HC, aT, bK, aPF, af, ak, qw, Uf, s, iU, la, aNi, akY, bQ, aDQ, BU, alt, aGo, mf, aoJ, hS, Kd, qb, av, F, oz, sO, fb, uQ, aF, ek, LB, boH, bE, Lb, aS, X); #c1(IGF1) c2(NP_001104753) c3(insulin-like growth factor I isoform 1 preproprotein) c4(42228, 42229, 42230) c5(42231, 42232, 42233, 42234, 42235, 42236, 42237, 42238, 42239, 42240, 42241, 42242, 42243, 42244, 42245, 42246, 42247, 42248, 42249, 42250, 42251, 42252, 42253, 42254, 42255, 42256, 42257, 42258, 42259, 42260, 42261) c6(42262, 42263, 42264, 42265, 42266, 42267, 42268, 42269) c7(T, ea, fd, ci, aY, GK, bv, aE, fm, dV, hu, avM, cp, li, yo, ZH, Ls, zE, iA, nB, cj, bOJ, FG, ht, cQ, aj, cd, Ak, ey, bl, fQ, fe, pm, QK, ok, dB, eD, bOE, Be, E, bOF, AN, jv, aG, aA, cz, bX, bOI, avO, q, kf, cy, rv, eu, bA, iF, nO, fO, cl, pb, bOG, rR, acW, ac, tW, mG, hr, cG, aDR, aps, bt, rA, bMo, iW, jh, KO, dG, ad, hX, yt, vJ, lQ, ahk, BO, cW, PE, ff, Of, dq, bz, OG, ag, aed, iu, if, e, bB, brq, ip, bc, Ur, hl, as, eF, cm, aiv, lt, vA, mx, vy, tV, bBT, Ht, dD, yG, n, nA, aje, cq, pe, eV, avP, A, RT, Rj, lb, hQ, yq, bP, PI, hL, at, ut, B, tq, vQ, AS, dA, eX, fF, GC, v, Do, V, eJ, Kx, UE, hW, ae, jf, ev, asy, bbs, aom, bu, t, aoy, aff, zJ, kP, wD, gX, zu, xb, hl, acl, RH, OR, zF, eq, hd, aop, LA, hD, vc, Dj, d, Z, HI, dr, D, gk, ll, gZ, Kj, y, ng, aC, fR, azf, yS, atj, al, bW, pX, M, K, eL, adT, BBQ, ago, apn, LZ, g, uC, iP, AM, U, uo, m, bBJ, sQ, OI, aw, NT, fg, aCQ, zP, JW, ft, uV, dx, CN, Og, W, anZ, wk, bo, it, ail, fG, qQ, aee, hz, j, sl, eA, J, XX, MR, lg, bBN, bq, kl, Y, LO, r, en, wA, iK, bN, Qi, Dy, c, kB, hc, dy, fD, gT, gx, sY, kh, id, bxK, aZ, bS, iJ, dP, fU, aet, tt, bKm, Yi, uX, lR, aus, xs, eH, cr, Fq, Bu, qa, pu, nb, AR, sG, bC, ei, vt, xg, G, pf, acz, fV, amj, bBb, mF, dY, il, iX, Ou, zQ, aW, hJ, eM, iT, aYS, gn, pw, ez, fr, ct, qN, rc, ke, cc, hw, rl, zf, h, jy, dL, Xh, Um, Ck, Dg, aq, kk, op, bJ, Qy, hR, arg, bL, eR, yk, hy, asA, uT, HC, aT, bK, aPF, af, ak, qw, Uf, s, iU, la, aNi, akY, bQ, aDQ, BU, alt, aGo, mf, aoJ, hS, Kd, qb, av, F, oz, sO, fb, uQ, aF, ek, LB, boH, bE, Lb, aS, X); #c1(IGF1) c2(NP_001104754) c3(insulin-like growth factor I isoform 2 preproprotein) c4(42270) c5(42271, 42272, 42273, 42274, 42275, 42276, 42277, 42278, 42279) c6(42280, 42281) c7(T, ea, fd, ci, aY, GK, bv, aE, fm, dV, hu, avM, cp, li, yo, ZH, Ls, zE, iA, nB, cj, bO J, FG, ht, cd, aj, cd, Ak, ey, bl, fQ, fe, pm, QK, ok, dB, eD, bOE, Be, E, bOF, AN, jv, aG, aA, cz, bX, bOI, avO, q, kf, cy, rv, eu, bA, iF, nO, fO, cl, pb, bOG, rR, acW, ac, tW, mG, hr, cG, aDR, aps, bt, rA, bMo, iW, jh, KO, dG, ad, hX, yt, vJ, lQ, ahk, BO, cW, PE, ff, Of, dq, bz, OG, ag, aed, iu, if, e, bB, brq, ip, bc, Ur, hl, as, eF, cm, aiv, lt, vA, mx, vy, tV, bBT, Ht, dD, yG, n, nA, aje, cq, pe, eV, avP, A, RT, Rj, lb, hQ, yq, bP, PI, hL, at, ut, B, tq, vQ, AS, dA, eX, fF, GC, v, Do, V, eJ, Kx, UE, hW, ae, jf, ev, asy, bbs, aom, bu, t, aoy, aff, zJ, kP, wD, gX, zu, xb, hl, acl, RH, OR, zF, eq, hd, aop, LA, hD, vc, Dj, d, Z, HI, dr, D, gk, ll, gZ, Kj, y, ng, aC, fR, azf, yS, atj, al, bW, pX, M, K, el, adT, BBQ, ago, apn, LZ, g, uC, iP, AM, U, uo, m, bBJ, sQ, Ol, aw, NT, fg, aCQ, zP, JW, ft, uV, dx, CN, Og, W, anZ, wk, bo, it, ail, fG, qQ, aee, hz, j, sl, eA, J, XX, MR, lg, bBN, bq, kl, Y, LO, r, en, wA, iK, bN, Qi, Dy, c, kB, hc, dy, fD, gT, gx, sY, kh, id, bxK, aZ, bS, iJ, dP, fU, aet, tt, bKm, Yi, uX, lR, aus, xs, eH, cr, Fq, Bu, qa, pu, nb, AR, sG, bC, ei, vt, xg, G, pf, acz, fV, amj, bBb, mF, dY, il, iX, Ou, zQ, aW, hJ, eM, iT, aYS, gn, pw, ez, fr, ct, qN, rc, ke, cc, hw, rl, zf, h, jy, dL, Xh, Um, Ck, Dg, aq, kk, op, bJ, Qy, hR, arg, bL, eR, yk, hy, asA, uT, HC, aT, bK, aPF, af, ak, qw, Uf, s, iU, la, aNi, akY, bQ, aDQ, BU, alt, aGo, mf, aoJ, hS, Kd, qb, av, F, oz, sO, fb, uQ, aF, ek, LB, boH, bE, Lb, aS, X); #c1(IGF1R) c2(NP_000866) c3(insulin-like growth factor I receptor isoform 1 precursor) c4(16652) c5(16653, 16654, 16655, 16656, 16657, 16658) c6( ) c7(T, aC, pf, hV, if, fd, bB, amj, ci, x, bv, aE, bOL, pX, aeO, iv, K, hu, eL, cp, aa, yo, zQ, JI, aW, Ls, LZ, nY, g, eF, zE, uC, cm, GR, zV, bw, eM, nB, iA, vA, cj, k, m, Xu, CE, iu, dD, ht, n, bOK, fr, qN, JW, cd, b, ft, jz, cc, zw, i, gd, jy, dL, dx, AQ, fe, aq, kp, yq, pm, QK, auC, op, oU, W, dB, hk, JQ, hL, HR, E, at, WL, it, B, bL, oe, aA, cz, hy, v, j, bX, q, er, qH, eA, BJ, mP, afQ, aT, bq, an, ae, af, eu, bA, jf, ak, qw, kl, s, Or, Y, LO, r, aNi, akY, en, hf, bu, t, cl, ik, ck, nM, ac, gX, hr, JL, mf, bt, xb, rA, VB, awY, sY, qb, Dd, acl, iW, ml, oH, Gn, eq, iJ, ad, dZ, hX, hd, ib, py, HD, vc, hP, fb, NP, d, ff, eH, cr, gl, aF, iR, bz, qa, es, Hw, Ew, bE, AR, pP, aef, Lb, ei, adg, lC, vt, X, y); #c1(IGF2) c2(NP_001121070) c3(insulin-like growth factor II isoform 2) c4(45152, 45153, 45154) c5(45155, 45156, 45157, 45158, 45159, 45160, 45161, 45162, 45163, 45164, 45165, 45166, 45167, 45168, 45169, 45170, 45171, 45172) c6( ) c7(G, T, aC, UH, fR, pf, ea, atj, e, al, if, aHx, eb, bB, ci, brq, ip, x, bv, lE, aE, pX, dV, JT, iv, DD, Ou, K, hu, eL, Qe, li, eG, yo, zQ, JI, aW, az, hl, Qm, g, as, eF, zE, uC, cm, Qt, bw, eM, nB, AM, iT, vA, ZT, k, m, pw, Xu, iu, bBP, bOJ, dD, gU, wu, aw, ht, fg, n, aj, fr, qN, cd, Vs, jz, no, hw, cc, aoc, TK, PD, aOM, bOO, jy, dL, Jn, h, uV, pl, A, CM, iw, hQ, ahJ, aq, fe, Hu, pm, bOP, op, bJ, iO, dB, hk, eD, PI, wk, bOR, KT, mg, hL, at, pg, bo, azr, aDE, bOQ, it, B, L, bL, vQ, jv, fG, oe, Lx, qQ, jx, fZ, aA, cz, xk, j, v, q, J, er, eA, PC, Zp, mj, amg, hW, aql, aT, cy, bq, an, ae, af, jf, ak, kl, wr, s, ND, vT, Qr, Y, ev, LO, iU, bOT, r, akY, en, bu, cl, t, ik, alp, bOS, bN, ck, Qi, QG, wg, kP, pO, alt, aGo, au, aJd, aps, bt, xb, yv, vi, hS, sY, kh, id, MH, aZ, iW, jN, ml, jh, dG, eq, iJ, ad, afb, hX, qC, Jm, av, iC, ot, oz, Ot, Hk, oW, wm, fb, nJ, d, BO, et, ff, eH, Ej, Lk, aF, iV, iR, adj, Bu, bz, dr, bOM, bF, es, gk, bON, mi, pc, bE, ll, Lb, Km, cP, ei, LD, y, X, jr); #c1(IGF2) c2(NP_001278791) c3(insulin-like growth factor II isoform 1 preproprotein) c4( ) c5(110088, 110089, 110090, 110091, 110092, 110093, 110094, 110095, 110096, 110097, 110098, 110099, 110100, 110101, 110102, 110103, 110104, 110105, 110106, 110107, 110108, 110109, 110110, 110111, 110112, 110113, 110114, 110115, 110116, 11017, 110118, 110119, 110120, 110121, 110122) c6( ) c7(G, T, aC, UH, FR, pf, ea, atj, e, al, if, aHx, eb, bB, ci, brq, ip, x, bv, lE, aE, pX, dV, JT, iv, DD, Ou, K, hu, eL, Qe, li, eG, yo, zQ, JI, aW, az, hl, Om, g, as, eF, zE, uC, cm, Qt, bw, eM, nB, AM, iT, vA, ZT, k, m, pw, Xu, iu, bBP, bOJ, dD, gU, wu, aw, ht, fg, n, aj, fr, qN, cd, Vs, jz, no, hw, cc, aoc, TK, PD, aOM, bOO, jy, dL, Jn, h, uV, pl, A, CM, iw, hQ, ahJ, aq, fe, Hu, pm, bOP, op, bJ, iO, dB, hk, eD, PI, wk, bOR, KT, mg, hl, at, pg, bo, azr, aDE, bOG, it, B, L, bL, vQ, jv, fG, oe, Lx, qQ, jx, fZ, aA, cz, xk, j, v, q, J, er, eA, PC, Zp, mj, amg, hW, aql, aT, cy, bq, an, ae, af, jf, ak, kl, wr, s, ND, vT, Qr, Y, ev, LO, iU, bOT, r, akY, en, bu, cl, t, ik, alp, bOS, bN, ck, Qi, QG, wg, kP, pD, alt, aGo, au, aJQ, aps, bt, xb, yv, vi, hS, sY, kh, id, MH, aZ, iW, jN, ml, jh, dG, eq, iJ, ad, afb, hX, qC, Jm, av, iC, ot, oz, Ot, Hk, oW, wm, fb, nJ, d, BO, et, ff, eH, Ej, Lk, aF, iV, iR, adj, Bu, bz, dr, bOM, bF, es, gk, bON, mi, pc, bE, ll, Lb, Km, cP, ei, LD, y, X, jr); #c1(IGF2BP1) c2(NP_001153895) c3(insulin-like growth factor 2 mRNA-binding protein 1 isoform 2) c4(61033) c5(61034, 61035, 61036, 61037, 61038, 61039) c6( ) c7(bL, T, dD, jh, bB, n, hy, v, eq, ci, ip, av, aT, nJ, cp, B, BN, aW, Y, yx, aq, iR, t, SE, dB, bE, X, qF, ei, ne); #c1(IGF2BP1) c2(NP_006537) c3(insulin-like growth factor 2 mRNA-binding protein 1 isoform 1) c4(166089) c5(166090, 166091, 166092, 166093, 166094, 166095) c6( ) c7(bL, T, dD, jh, bB, n, hy, v, eq, ci, ip, av, aT, nJ, cp, B, BN, aW, Y, yx, aq, iR, t, SE, dB, bE, X, qF, ei, ne); #c1(IGF2BP2) c2(NP_001007226) c3(insulin-like growth factor 2 mRNA-binding protein 2 isoform b) c4(22572) c5(22573, 22574, 22575, 22576, 22577, 22578) c6( ) c7(bL, T, dD, aC, e, jh, aj, dG, j, v, eq, GS, cd, q, J, ke, h, d, zR, r, au, mg, mf, lS, ei); #c1(IGF2BP2) c2(NP_001278801) c3(insulin-like growth factor 2 mRNA-binding protein 2 isoform d) c4(110132) c5(110133, 110134, 110135, 110136, 110137, 110138) c6( ) c7(bL, T, dD, aC, e, jh, aj, dG, j, v, eq, GS, cd, q, J, ke, h, d, zR, r, au, mg, mf, lS, ei); #c1(IGF2BP2) c2(NP_006539) c3(insulin-like growth factor 2 mRNA-binding protein 2 isoform a) c4(166103) c5(166104, 166105, 166106, 166107, 166108, 166109) c6( ) c7(bL, T, dD, aC, e, jh, aj, dG, j, v, eq, GS, cd, q, J, ke, h, d, zR, r, au, mg, mf, lS, ei); #c1(IGF2BP3) c2(NP_006538) c3(insulin-like growth factor 2 mRNA-binding protein 3) c4(166096) c5(166097, 166098, 166099, 166100, 166101, 166102) c6( ) c7(bL, gx, T, Mb, KR, jh, v, oH, ci, bX, fr, q, iu, hd, aE, aT, dL, eL, aol, aW, Y, AQ, eH, LZ, r, nY, en, cl, g, as, eF, kP, nG, aep, hl, qF, ei, B); #c1(IGF2R) c2(NP_000867) c3(cation-independent mannose-6-phosphate receptor precursor) c4(16659) c5(16660, 16661, 16662, 16663, 16664, 16665) c6( ) c7(bL, sY, T, aC, eR, fR, jp, ZI, iW, aw, ht, al, hE, aA, ml, v, ci, vY, V, q, ad, er, bOV, Jm, av, jz, aE, il, awd, yg, aT, iv, h, dL, K, ux, an, ex, ae, af, CB, aa, ak, kl, zQ, A, s, aW, Y, ff, az, eH, r, aq, lo, Ej, aF, bOU, en, cl, g, t, uC, bz, zZ, eF, iK, iR, bP, c, nB, dB, Qu, ag, eM, gX, cj, at, Hb, m, fD, sT, B); #c1(IGFALS) c2(NP_001139478) c3(insulin-like growth factor-binding protein complex acid labile subunit isoform 1 precursor) c4(59400) c5(59401, 59402, 59403, 59404, 59405, 59406, 59407, 59408, 59409, 59410, 59411, 59412) c6( ) c7(bLx, dt, mr, Ol, rf, bBX, Vd, BOX, PJ, ad, PW, Hx, Gt, no, WY, aet, aT, kf, bBN, jy, h, ama, K, bl, an, wn, af, aIL, ak, zQ, apl, axR, PE, Zf, aav, axW, alR, TA, yy, aaE, bOW, bu, QK, xw, bb, ac, c, apq, U, bel, at, Lb, lM, xb, L); #c1(IGFALS) c2(NP_004961) c3(insulin-like growth factor-binding protein complex acid labile subunit isoform 2 precursor) c4(151500) c5( ) c6( ) c7(bLx, dt, mr, Ol, rf, bBX, Vd, bOX, PJ, ad, PW, Hx, Gt, no, WY, aet, aT, kf, bBN, jy, h, ama, K, bl, an, wn, af, aIL, ak, zQ, apl, axR, PE, Zf, aav, axW, alR, TQ, yy, aaE, bOW, bu, QK, xw, bb, ac, c, apq, U, bel, at, Lb, IM, xb, L); #c1(IGFBP1) c2(NP_000587) c3(insulin-like growth factor-binding protein 1 precursor) c4(13257) c5(13258, 13259, 13260, 13261, 13262, 13263) c6( ) c7(bL, aC, dD, fV, ea, e, al, jN, iW, aA, eb, ez, aj, dG, j, v, eq, qN, fd, aY, Eh, eA, J, ad, Vs, dP, x, ft, aE, Xc, iX, pX, MP, dV, aT, jy, bOY, dL, vc, ey, ae, af, bc, kl, zQ, ZH, s, CN, PE, aW, Rr, hl, LO, r, fQ, aq, eH, EH, aF, Fq, cl, g, t, zE, uC, pm, op, gk, mi, eM, AM, AR, vA, X, hr, fP, hL, E, at, mf, m, ei, gT, B); #c1(IGFBP2) c2(NP_000588) c3(insulin-like growth factor-binding protein 2 precursor) c4(13264) c5(13265, 13266, 13267, 13268, 13269, 13270) c6( ) c7(T, bO, fd, bB, ci, mF, ip, x, bv, iv, DD, aW, hl, g, as, cm, eM, vA, Zn, k, m, aCo, Vj, dD, qy, aj, fr, b, ft, PD, Lv, dL, dx, A, iw, fQ, cM, fs, op, hq, iO, bP, cA, dB, hL, at, bL, aA, cz, hy, v, so, q, J, eA, mj, qK, aT, bK, ae, af, ne, jf, iF, Uf, s, Y, r, qL, en, cl, t, ck, sS, gT, bt, LS, aZ, aCm, Fb, eq, hF, Fc, iJ, ad, iC, av, uQ, d, BN, ff, aF, bz, bE, Lb, ei, i, X); #c1(IGFBP3) c2(NP_000589) c3(insulin-like growth factor-binding protein 3 isoform b precursor) c4(13271, 13272, 13273, 13274, 13275, 13276, 13277, 13278) c5(13279, 13280, 13281, 13282, 13283, 13284, 13285, 13286, 13287, 13288, 13289, 13290, 13291, 13292, 13293, 13294, 13295, 13296, 13297, 13298, 13299, 13300, 13301, 13302, 13303, 13304, 13305, 13306, 13307, 13308, 13309, 13310, 13311, 13312, 13313, 13314, 13315, 13316, 13317, 13318, 13319, 13320, 13321, 13322, 13323, 13324, 13325, 13326, 13327, 13328, 13329, 13330, 13331, 13332, 13333, 13334, 13335, 13336, 13337, 13338, 13339, 13340, 13341, 13342) c6(13343, 13344, 13345, 13346, 13347, 13348, 13349, 13350, 13351, 13352, 13353, 13354, 13355, 13356, 13357, 13358) c7(gh, G, T, bM, aC, fR, pf, atj, e, al, if, fd, bB, amj, ci, XW, dV, K, hu, eL, cp, azu, zQ, JI, aW, hl, LZ, g, as, eF, zE, bw, aAW, lt, eM, iA, iT, vA, uo, m, Xu, Vj, dD, sl, ht, NT, n, aj, fr, qN, JW, ai, ft, PD, jy, dL, hi, Ck, yi, Rj, asl, fe, aq, kp, cM, op, bJ, iO, bP, dB, anZ, PI, bo, hL, at, ut, it, B, bL, dA, fG, aA, hz, hy, v, j, asA, AO, q, J, eA, XX, cF, MR, lg, aT, cy, bq, ae, af, jf, wr, s, Y, LO, r, akY, en, bu, cl, t, ik, pb, bN, ck, Di, acW, kP, ac, alt, mf, aoJ, dy, anD, xb, hS, kh, id, bxK, qb, iW, OR, Fb, zF, eq, gb, Pm, ad, dP, av, iC, py, vJ, sD, vc, lQ, fb, uQ, d, lR, PE, ff, eH, eZ, aF, Fq, iR, Bu, bz, HI, LB, es, bE, ll, ei, vt, X); #c1(IGFBP3) c2(NP_001013416) c3(insulin-like growth factor-binding protein 3 isoform a precursor) c4(25508) c5(25509, 25510, 25511, 25512, 25513, 25514, 25515, 25516) c6(25517, 25518) c7(gh, G, T, bM, aC, fR, pf, atj, e, al, if, fd, bB, amj, ci, XW, dV, K, hu, eL, cp, azu, zQ, JI, aW, hl, LZ, g, as, eF, zE, bw, aAW, lt, eM, iA, iT, vA, uo, m, Xu, Vj, dD, sQ, ht, NT, n, aj, fr, qN, JW, ai, ft, PD, jy, dL, hi, Ck, yi, Rj, asL, fe, aq, kp, cM, op, bJ, iO, bP, dB, anZ, PI, bo, hL, at, ut, it, B, bL, dA, fG, aA, hz, hy, v, j, asA, AD, q, J, eA, XX, cF, MR, lg, aT, cy, bq, ae, af, jf, wr, s, Y, LO, r, akY, en, bu, cl, t, iK, pb, bN, ck, Qi, acW, kP, ac, aJt, mf, aoJ, dy, anD, xb, hS, kh, id, bxK, qb, iW, OR, Fb, zF, eq, gb, Pm, ad, dP, av, iC, py, vJ, sO, vc, lQ, fb, uQ, d, lR, PE, ff, eH, eZ, aF, Fq, iR, Bu, bz, HI, LB, es, bE, ll, ei, vt, X); #c1(IGFBP4) c2(NP_001543) c3(insulin-like growth factor-binding protein 4 precursor) c4(118736, (18737) c5(118738, 118739, 118740, 118741, 118742, 118743, 118744, 118745, 118746, 118747, 118748, 118749) c6( ) c7(T, bB, Fb, fr, eA, q, ad, Vs, aT, ae, af, uQ, d, nO, aW, Y, r, aF, g, cl, as, cm, t, eM, bE, hL, zu, y); #c1(IGFBP5) c2(NP_000590) c3(insulin-like growth factor-binding protein 5 precursor) c4(13359) c5(13360, 13361, 13362, 13363, 13364, 13365, 13366, 13367) c6( ) c7(bL, T, aC, id, fG, qy, eb, n, aCm, aj, hy, ci, qN, fr, q, J, ad, ip, XX, Vp, iC, Db, aT, PD, dL, K, fb, ae, af, A, d, BN, B, aW, ff, eH, r, en, cl, g, t, eF, bz, pb, cm, ik, LB, nB, hL, at, aS, aCo, ei, y, ne); #c1(IGFBP6) c2(NP_002169) c3(insulin-like growth factor-binding protein 6 precursor) c4(126246, 126247, 126248) c5(126249, 126250, 126251, 126252, 126253, 126254, 126255, 126256, 126257, 126258, 126259, 126260, 126261, 126262, 126263, 126264, 126265, 126266, 126267, 126268, 126269, 126270, 126271, 126272) c6(126273, 126274, 126275, 126276, 126277, 126278) c7(gh, T, aZ, jN, bB, fr, eJ, eA, ad, ip, dP, bv, aT, dL, hP, ae, af, hl, t, cm, LS, eM, bE, bo, at, azr, bBP); #c1(IGFBP7) c2(NP_001240764) c3(insulin-like growth factor-binding protein 7 isoform 2 precursor) c4(88763) c5(88764, 88765, 88766, 88767, 88768, 88769) c6( ) c7(T, id, pf, oe, aA, bB, aj, hy, bzl, ci, v, fr, rP, hF, cd, q, ad, hX, dP, eA, av, x, aE, bPa, kH, aT, h, hn, ey, bOZ, ae, eL, cp, uX, d, aW, Y, ev, ff, blP, r, aF, en, cl, g, cm, iK, bz, op, Hp, ck, es, W, eM, nB, bE, iA, vA, hL, JH, k, hE, bt, B); #c1(IGFBP7) c2(NP_001544) c3(insulin-like growth factor-binding protein 7 isoform 1 precursor) c4(118750) c5(118751, 118752, 118753, 118754, 118755, 118756) c6( ) c7(T, id, pf, oe, aA, bB, aj, hy, bzl, ci, v, fr, rP, hF, cd, q, ad, hX, dP, eA, av, x, aE, bPa, KH, aT, h, hn, ey, bOZ, ae, eL, cp, uX, d, aW, Y, ev, ff, blP, r, aF, en, cl, g, cm, ik, bz, op, Hp, ck, es, W, eM, nB, bE, iA, vA, hL, JH, k, hE, bt, B); #c1(IGFBPL1) c2(NP_001007564) c3(insulin-like growth factor-binding protein-like 1 precursor) c4(22812) c5(22813, 22814, 22815, 22816, 22817, 22818, 22819, 22820, 22821) c6(22822, 22823) c7(aW, T, cz, ci, aT); #c1(IGFL1) c2(NP_940943) c3(insulin growth factor-like family member 1 precursor) c4(260174) c5(260175, 260176, 260177, 260178, 260179, 260180) c6( ) c7(IQ, cr); #c1(IGFLRI) c2(NP_078936) c3(IGF-like family receptor 1 precursor) c4(216180, 216181) c5(216182, 216183, 216184, 216185, 216186, 216187, 216188, 216189, 216190, 216191, 216192, 216193, 216194) c6( ) c7(IL); #c1(IGHMBP2) c2(NP_002171) c3(DNA-binding protein SMUBP-2) c4(126279) c5(126280, 126281, 126282, 126283, 126284, 126285, 126286) c6( ) c7(Oq, aPN, apq, apl, aW, bT, Ol, Ad, aT, kf, bej, oj, U, ey, fu, bX, EZ, fq, aof); #c1(IGJ) c2(NP_653247) c3(immunoglobulin J chain precursor) c4(239311, 239312, 239313) c5(239314, 239315, 239316, 239317, 239318, 239319, 239320, 239321, 239322, 239323, 239324, 239325, 239326, 239327, 239328, 239329, 239330, 239331, 239332, 239333, 239334, 239335) c6( ) c7(eE); #c1(IGLL1) c2(NP_064455) c3(immunoglobulin lambda-like polypeptide 1 isoform a precursor) c4(202572) c5(202573, 202574, 202575, 202576, 202577, 202578, 202579) c6( ) c7(bPb, bPc, cj, vo, Ze, asE); #c1(IGLL1) c2(NP_690594) c3(immunoglobulin lambda-like polypeptide 1 isoform b precursor) c4(244878) c5(244879, 244880, 244881, 244882, 244883) c6( ) c7(bPb, bPc, cj, vo, Ze, asE); #c1(IGSF1) c2(NP_001164432) c3(immunoglobulin superfamily member 1 isoform 3 precursor) c4(66948) c5(66949, 66950, 66951, 66952, 66953, 66954) c6( ) c7(T, aE, AO, qt, aW, RN, r, aT, ci, zW, zy, brw, bPd, eA); #c1(IGSF1) c2(NP_001164433) c3(immunoglobulin superfamily member 1 isoform 4) c4(66955) c5(66956, 66957, 66958, 66959, 66960, 66961, 66962, 66963, 66964, 66965) c6( ) c7(T, aE, AO, qt, aW, RN, r, aT, ci, zW, zy, brw, bPd, eA); #c1(IGSF1) c2(NP_001546) c3(immunoglobulin superfamily member 1 isoform 1 precursor) c4(118757) c5(118758, 118759, 118760, 118761, (18762, 118763) c6( ) c7(T, aE, AO, qt, aW, RN, r, aT, ci, zW, zy, brw, bPd, eA); #c1(IGSF1) c2(NP_991402) c3(immunoglobulin superfamily member 1 isoform 2 precursor) c4(263447) c5(263448, 263449, 263450, 263451, 263452, 263453, 263454, 263455, 263456, 263457) c6( ) c7(T, aE, AO, qt, aW, RN, r, aT, ci, zW, zy, brw, bPd, eA); #c1(IGSF1) c2(XP_011529636) c3(immunoglobulin superfamily member 1 isoform X4) c4( ) c5(325285) c6( ) c7(T, aE, AO, qt, aW, RN, r, aT, ci, zW, zy, brw, bPd, eA); #c1(IGSF10) c2(NP_849144) c3(immunoglobulin superfamily member 10 isoform 1 precursor) c4(254257, 254258) c5(254259, 254260, 254261, 254262, 254263, 254264, 254265, 254266, 254267, 254268, 254269, 254270, 254271, 254272, 254273, 254274, 254275) c6(254276, 254277, 254278, 254279) c7(cd); #c1(IGSF11) c2(NP_001015887) c3(immunoglobulin superfamily member Il isoform b precursor) c4(26238) c5(26239, 26240, 26241, 26242, 26243, 26244) c6( ) c7(T, aF, B, bu, aA); #c1(IGSF11) c2(NP_689751) c3(immunoglobulin superfamily member Il isoform a precursor) c4(243848) c5(243849, 243850, 243851, 243852, 243853, 243854, 243855, 243856, 243857, 243858) c6( ) c7(T, aF, B, bu, aA); #c1(IGSF5) c2(NP_001073913) c3(immunoglobulin superfamily member 5) c4(36506) c5(36507, 36508, 36509, 36510, 36511, 36512) c6( ) c7(j, ac, ad, an); #c1(IGSF6) c2(NP_005840) c3(immunoglobulin superfamily member 6 precursor) c4(159799) c5(159800, 159801, 159802, 159803, 159804, 159805) c6( ) c7(fJ); #c1(IGSF8) c2(NP_001193594) c3(immunoglobulin superfamily member 8 precursor) c4(82309, 82310) c5(82311, 82312, 82313, 82314, 82315, 82316, 82317, 82318, 82319, 82320) c6( ) c7(eA, jx, d, ad); #c1(IGSF8) c2(XP_006711694) c3(immunoglobulin superfamily member 8 isoform X2) c4(286219) c5(286220, 286221, 286222, 286223, 286224, 286225, 286226, 286227, 286228, 286229, 286230, 286231) c6( ) c7(eA, jx, d, ad); #c1(IHH) c2(NP_002172) c3(indian hedgehog protein precursor) c4(126287) c5(126288, 126289, 126290, 126291, 126292, (26293) c6( ) c7(T, kh, aC, rV, fg, aA, bB, zU, yn, dP, zw, MS, aE, aT, bpa, ahE, bPf, aW, r, atf, kp, aF, ahl, boW, dr, atb, zV, bF, bE, apy, zG, bPe, vt, xb, bwD); #c1(IK) c2(NP_006074) c3(protein Red) c4(161673, 161674) c5(161675, 161676, 161677, 161678, 161679, 161680, 161681, 161682, 161683, 161684, 161685, 161686) c6( ) c7(aCi, NU, mA, hp, cF); #c1(IKBKAP) c2(NP_003631) c3(elongator complex protein 1) c4(139716, 139717) c5(139718, 139719, 139720, 139721, 139722, 139723) c6( ) c7(bM, Dw, ahW, aFa, pl, bX, pM, RD, aqJ, ahL, rO, Ds, af, sp, xs, uY, ahl, cm, ps, mx, m, bD, y, sH); #c1(IKBKB) c2(NP_001177649) c3(inhibitor of nuclear factor kappa-B kinase subunit beta isoform 2) c4(72810) c5(72811, 72812, 72813, 72814, 72815, 72816, 72817, 72818, 72819, 72820, 72821, 72822) c6( ) c7(bPg, bL, T, dD, bM, dA, Dw, hl, ea, iW, jx, jh, aA, cz, fd, eq, q, ad, eA, av, KH, Lv, aT, Ds, h, bl, ji, bq, d, B, je, aW, Y, ff, fO, fc, eZ, r, CF, Pr, vs, kp, en, g, bz, es, eU, iM, eM, iA, gn, cP, fD, y, xg, X); #c1(IKBKB) c2(NP_001229707) c3(inhibitor of nuclear factor kappa-B kinase subunit beta isoform 5) c4(84855, 84856) c5(84857, 84858, 84859, 84860, 84861, 84862) c6( ) c7(bPg, bL, T, dD, bM, dA, Dw, hl, ea, iW, jx, jh, aA, cz, fd, eq, q, ad, eA, av, KH, Lv, aT, Ds, h, bl, ji, bq, d, B, je, aW, Y, ff, fO, fc, eZ, r, CF, Pr, vs, kp, en, g, bz, es, eU, iM, eM, iA, gn, cP, fD, y, xg, X); #c1(IKBKB) c2(NP_001547) c3(inhibitor of nuclear factor kappa-B kinase subunit beta isoform 1) c4(118764) c5(118765, 118766, 118767, 118768, 118769, 118770) c6( ) c7(bPg, bL, T, dD, bM, dA, Dw, hl, ea, iW, jx, jh, aA, cz, fd, eq, q, ad, eA, av, KH, Lv, aT, Ds, h, bl, ji, bq, d, B, je, aW, Y, ff, fO, fc, eZ, r, CF, Pr, vs, kp, en, g, bz, es, eU, iM, eM, iA, gn, cP, fD, y, xg, X); #c1(IKBKE) c2(NP_001180250) c3(inhibitor of nuclear factor kappa-B kinase subunit epsilon isoform 3) c4( ) c5(73561, 73562, 73563, 73564, 73565, 73566, 73567) c6( ) c7(T, tb, fR, eM, aW, ff, ex, bO, aT, eZ, aiT, eV, ww, en, y, eA, afu); #c1(IKBKE) c2(NP_054721) c3(inhibitor of nuclear factor kappa-B kinase subunit epsilon isoform 1) c4(177350, 177351) c5(177352, 177353, 177354, 177355, 177356, 177357, 177358) c6( ) c7(T, tb, fR, eM, aW, ff, ex, bO, aT, eZ, aiT, eV, ww, en, y, eA, afu); #c1(IKBKG) c2(NP_001093326) c3(NF-kappa-B essential modulator isoform b) c4(40247) c5(40248, 40249, 40250, 40251, 40252, 40253, 40254, 40255, 40256, 40257) c6( ) c7(oO, Dw, agx, dD, bPk, BF, aml, uE, agz, eq, hF, aLI, Oe, av, bPh, bjA, bjv, oj, aT, Vv, cy, bOp, K, lQ, ji, hH, Cw, aW, oV, zb, CF, bPj, lo, bw, dr, afg, ck, bjx, bPI, aGV, bPi, cP, UF, HE, bt, zA, B); #c1(IKBKG) c2(NP_001138727) c3(NF-kappa-B essential modulator isoform c) c4(57454) c5(57455, 57456, 57457, 57458, 57459, 57460) c6( ) c7(oO, Dw, agx, dD, bPk, BF, aml, uE, agz, eq, hF, aLI, Oe, av, bPh, bjA, bjv, oj, aT, Vv, cy, bOp, K, lQ, ji, hH, Cw, aW, oV, zb, CF, bPj, lo, bw, dr, afg, ck, bjx, bPI, aGV, bPi, cP, UF, hE, bt, zA, B); #c1(IKBKG) c2(XP_005274817) c3(NF-kappa-B essential modulator isoform X2) c4( ) c5(285476) c6( ) c7(oO, Dw, agx, dD, bPk, BF, aml, uE, agz, eq, hF, aLI, Oe, av, bPh, bjA, bjv, oj, aT, Vv, cy, bOp, K, lQ, ji, hH, Cw, aW, oV, zb, CF, bPj, lo, bw, dr, afg, ck, bjx, bPI, aGV, bPi, cP, UF, hE, bt, zA, B); #c1(IKBKG) c2(XP_005274818) c3(NF-kappa-B essential modulator isoform X3) c4( ) c5(285477, 285478) c6( ) c7(oO, Dw, agx, dD, bPk, BF, aml, uE, agz, eq, hF, aLI, Oe, av, bPh, bjA, bjv, oj, aT, Vv, cy, bOp, K, lQ, ji, hH, Cw, aW, oV, zb, CF, bPj, lo, bw, dr, afg, ck, bjx, bPI, aGV, bPi, cP, UF, hE, bt, zA, B); #c1(IKBKG) c2(XP_005274821) c3(NF-kappa-B essential modulator isoform X6) c4(285479) c5(285480, 285481, 285482, 285483, 285484) c6( ) c7(oO, Dw, agx, dD, bPk, BF, aml, uE, agz, eq, hF, aLI, Oe, av, bPh, bjA, bjv, oj, aT, Vv, cy, bOp, K, lQ, ji, hH, Cw, aW, oV, zb, CF, bPj, lo, bw, dr, afg, ck, bjx, bPI, aGV, bPi, cP, UF, hE, bt, zA, B); #c1(IKBKG) c2(XP_011529505) c3(NF-kappa-B essential modulator isoform X4) c4(325001) c5(325002, 325003, 325004, 325005, 325006, 325007) c6( ) c7(oO, Dw, agx, dD, bPk, BF, aml, uE, agz, eq, hF, aLI, Oe, av, bPh, bjA, bjv, oj, aT, Vv, cy, bOp, K, lQ, ji, hH, Cw, aW, oV, zb, CF, bPj, lo, bw, dr, afg, ck, bjx, bPI, aGV, bPi, cP, UF, hE, bt, zA, B); #c1(IKBKG) c2(XP_011529507) c3(NF-kappa-B essential modulator isoform X1) c4(325008, 325009, 325010, 325011) c5(325012, 325013, 325014, 325015, 325016, 325017, 325018, 325019, 325020, 325021, 325022, 325023, 325024, 325025, 325026, 325027, 325028, 325029, 325030, 325031, 325032, 325033, 325034, 325035) c6( ) c7(oO, Dw, agx, dD, bPk, BF, aml, uE, agz, eq, hF, aLI, Oe, av, bPh, bjA, bjv, oj, aT, Vv, cy, bOp, K, lQ, ji, hH, Cw, aW, oV, zb, CF, bPj, lo, bw, dr, afg, ck, bjx, bPI, aGV, bPi, cP, UF, HE, bt, zA, B); #c1(IKBKG) c2(XP_011529509) c3(NF-kappa-B essential modulator isoform X7) c4( ) c5(325036, 325037, 325038, 325039, 325040, 325041) c6( ) c7(oO, Dw, agx, dD, bPk, BF, aml, uE, agz, eq, hF, aLI, Oe, av, bPh, bjA, bjv, oj, aT, Vv, cy, bOp, K, lQ, ji, hH, Cw, aW, oV, zb, CF, bPj, lo, bw, dr, afg, ck, bjx, bPI, aGV, bPi, cP, UF, hE, bt, zA, B); #c1(IKZF1) c2(NP_001207697) c3(DNA-binding protein Ikaros isoform 5) c4(83718) c5(83719, 83720, 83721, 83722, 83723, 83724) c6( ) c7(T, ow, al, jh, fd, n, oZ, hF, q, x, fx, Bj, ox, iL, wC, A, iU, eZ, r, fe, hf, iV, ck, ou, ll, bt, k, WL, ne); #c1(IKZF1) c2(NP_001207700) c3(DNA-binding protein Ikaros isoform 8) c4(83725) c5(83726, 83727, 83728, 83729, 83730, 83731) c6( ) c7(T, ow, al, jh, fd, n, oZ, hF, q, x, fx, Bj, ox, iL, wC, A, iU, eZ, r, fe, hf, iV, ck, ou, ll, bt, k, WL, ne); #c1(IKZF1) c2(NP_001278766) c3(DNA-binding protein Ikaros isoform 2) c4( ) c5(110058, 110059, 110060, 110061, 110062, 110063, 110064, 110065, 110066, 110067, 110068, 110069, 110070) c6( ) c7(T, ow, al, jh, fd, n, oZ, hF, q, x, fx, Bj, ox, iL, wC, A, iU, eZ, r, fe, hf, iV, ck, ou, ll, bt, k, WL, ne); #c1(IKZF1) c2(NP_001278767) c3(DNA-binding protein Ikaros isoform 3) c4( ) c5(110071, 110072, 110073, 110074, 110075, 110076, 110077, 110078) c6( ) c7(T, ow, al, jh, fd, n, oZ, hF, q, x, fX, Bj, ox, iL, wC, A, iU, eZ, r, fe, hf, iV, ck, ou, ll, bt, k, WL, ne); #c1(IKZF1) c2(NP_001278769) c3(DNA-binding protein Ikaros isoform Ik-6) c4(110079) c5(110080, 110081, 110082, 110083, 110084, 110085) c6( ) c7(T, ow, al, jh, fd, n, oZ, hF, q, x, fx, Bj, ox, iL, wC, A, iU, eZ, r, fe, hf, iV, ck, ou, ll, bt, k, WL, ne); #c1(IKZF1) c2(NP_006051) c3(DNA-binding protein Ikaros isoform 1) c4( ) c5(161468, 161469, 161470, 161471, 161472) c6( ) c7(T, ow, al, jh, fd, n, oZ, hF, q, x, fx, Bj, ox, iL, wC, A, iU, eZ, r, fe, hf, iV, ck, ou, ll, bt, k, WL, ne); #c1(IKZF1) c2(XP_011513363) c3(DNA-binding protein Ikaros isoform X6) c4(305598) c5(305599, 305600) c6( ) c7(T, ow, al, jh, fd, n, oZ, hF, q, x, fx, Bj, ox, iL, wC, A, iU, eZ, r, fe, hf, iV, ck, ou, ll, bt, k, WL, ne); #c1(IKZF1) c2(XP_011513372) c3(DNA-binding protein Ikaros isoform X9) c4(305601, 305602) c5(305603, 305604, 305605) c6( ) c7(T, ow, al, jh, fd, n, oZ, hF, q, x, fx, Bj, ox, iL, wC, A, iU, eZ, r, fe, hf, iV, ck, ou, ll, bt, k, WL, ne); #c1(IKZF1) c2(XP_011513374) c3(DNA-binding protein Ikaros isoform X1I) c4(305606) c5(305607, 305608) c6( ) c7(T, ow, al, jh, fd, n, oZ, hF, q, x, fx, Bj, ox, iL, wC, A, iU, eZ, r, fe, hf, iV, ck, ou, ll, bt, k, WL, ne); #c1(IKZF2) c2(NP_001072994) c3(zinc finger protein Helios isoform 2) c4(35534, 35535) c5(35536, 35537, 35538, 35539, 35540, 35541, 35542, 35543, 35544, 35545, 35546) c6( ) c7(ck, aW, oe, jh, aT, k); #c1(IKZF2) c2(NP_057344) c3(zinc finger protein Helios isoform 1) c4(190200, 190201, 190202) c5(190203, 190204, 190205, 190206, 190207, 190208, 190209, 190210, 190211, 190212, 190213, 190214, 190215, 190216, 190217, 190218, 190219, 190220, 190221, 190222, 190223) c6( ) c7(ck, aW, oe, jh, aT, k); #c1(IKZF2) c2(XP_005246443) c3(zinc finger protein Helios isoform X6) c4(267147) c5(267148) c6( ) c7(ck, aW, oe, jh, aT, k); #c1(IKZF2) c2(XP_011509115) c3(zinc finger protein Helios isoform X4) c4(299697, 299698) c5(299699, 299700, 299701, 299702, 299703, 299704, 299705, 299706, 299707, 299708) c6( ) c7(ck, aW, oe, jh, aT, k); #c1(IKZF2) c2(XP_011509117) c3(zinc finger protein Helios isoform X5) c4(299709) c5( ) c6( ) c7(ck, aW, oe, jh, aT, k); #c1(IKZF2) c2(XP_011509120) c3(zinc finger protein Helios isoform X9) c4( ) c5(299710, 299711, 299712, 299713, 299714, 299715, 299716, 299717) c6( ) c7(ck, aW, oe, jh, aT, k); #c1(IKZF2) c2(XP_011509121) c3(zinc finger protein Helios isoform X10) c4(299718, 299719) c5(299720, 299721, 299722, 299723, 299724, 299725, 299726, 299727, 299728, 299729, 299730, 299731, 299732, 299733, 299734, 299735, 299736, 299737, 299738, 299739, 299740, 299741, 299742, 299743) c6( ) c7(ck, aW, oe, jh, aT, k); #c1(IKZF3) c2(NP_001244337) c3(zinc finger protein Aiolos isoform 7) c4(92203, 92204) c5(92205, 92206, 92207, 92208, 92209, 92210, 92211, 92212, 92213, 92214, 92215, 92216) c6( ) c7(ck, bM, A, aE, aW, NT, gd, cj, gw, aT, r, eZ, fd, k, jh, y, q, bc); #c1(IKZF3) c2(NP_001244338) c3(zinc finger protein Aiolos isoform 8) c4(92217) c5(92218, 92219, 92220, 92221, 92222, 92223) c6( ) c7(ck, bM, A, aE, aW, NT, gd, cj, gw, aT, r, eZ, fd, k, jh, y, q, bc); #c1(IKZF3) c2(NP_001244339) c3(zinc finger protein Aiolos isoform 9) c4(92224) c5(92225, 92226, 92227, 92228, 92229, 92230) c6( ) c7(ck, bM, A, aE, aW, NT, gd, cj, gw, aT, r, eZ, fd, k, jh, y, q, bc); #c1(IKZF3) c2(NP_001244340) c3(zinc finger protein Aiolos isoform 10) c4(92231) c5(92232, 92233, 92234, 92235, 92236, 92237) c6( ) c7(ck, bM, A, aE, aW, NT, gd, cj, gw, al, r, eZ, fd, k, jh, y, q, bc); #c1(IKZF3) c2(NP_001244341) c3(zinc finger protein Aiolos isoform 11) c4(92238) c5(92239, 92240, 92241, 92242, 92243, 92244) c6( ) c7(ck, bM, A, aE, aW, NT, gd, cj, gw, aT, r, eZ, fd, k, jh, y, q, bc); #c1(IKZF3) c2(NP_001244342) c3(zinc finger protein Aiolos isoform 12) c4(92245) c5(92246, 92247, 92248, 92249, 92250, 92251) c6( ) c7(ck, bM, A, aE, aW, NT, gd, cj, gw, aT, r, eZ, fd, k, jh, y, q, bc); #c1(IKZF3) c2(NP_001244343) c3(zinc finger protein Aiolos isoform 13) c4(92252) c5(92253, 92254, 92255, 92256, 92257, 92258) c6( ) c7(ck, bM, A, aE, aW, NT, gd, cj, gw, aT, r, eZ, fd, k, jh, y, q, bc); #c1(IKZF3) c2(NP_036613) c3(zinc finger protein Aiolos isoform 1) c4(174840) c5(174841, 174842, 174843, 174844, 174845, 174846, 174847) c6( ) c7(ck, bM, A, aE, aW, NT, gd, cj, gw, aT, r, eZ, fd, k, jh, y, q, bc); #c1(IKZF3) c2(NP_899051) c3(zinc finger protein Aiolos isoform 2) c4(257865) c5(257866, 257867, 257868, 257869, 257870, 257871) c6( ) c7(ck, bM, A, aE, aW, NT, gd, cj, gw, aT, r, eZ, fd, k, jh, y, q, bc); #c1(IKZF3) c2(NP_899052) c3(zinc finger protein Aiolos isoform 3) c4(257872) c5(257873, 257874, 257875, 257876, 257877, 257878) c6( ) c7(ck, bM, A, aE, aW, NT, gd, cj, gw, aT, r, eZ, fd, k, jh, y, q, bc); #c1(IKZF3) c2(NP_899053) c3(zinc finger protein Aiolos isoform 4) c4(257879) c5(257880, 257881, 257882, 257883, 257884, 257885) c6( ) c7(ck, bM, A, aE, aW, NT, gd, cj, gw, aT, r, eZ, fd, k, jh, y, q, bc); #c1(IKZF3) c2(NP_899054) c3(zinc finger protein Aiolos isoform 5) c4(257886) c5(257887, 257888, 257889, 257890, 257891, 257892) c6( ) c7(ck, bM, A, aE, aW, NT, gd, cj, gw, aT, r, eZ, fd, k, jh, y, q, bc); #c1(IKZF3) c2(NP_899055) c3(zinc finger protein Aiolos isoform 6) c4(257893) c5(257894, 257895, 257896, 257897, 257898, 257899) c6( ) c7(ck, bM, A, aE, aW, NT, gd, cj, gw, aT, r, eZ, fd, k, jh, y, q, bc); #c1(IKZF4) c2(NP_071910) c3(zinc finger protein Eos) c4(212259) c5(212260, 212261, 212262, 212263, 212264, 212265, 212266, 212267, 212268, 212269, 212270, 212271, 212272, 212273, 212274, 212275, 212276, 212277, 212278, 212279, 212280, 212281) c6( ) c7(Qi, bEg, A, OW, qj); #c1(IKZF4) c2(XP_011536970) c3(zinc finger protein Eos isoform X3) c4(334663) c5( ) c6( ) c7(Qi, bEg, A, OW, qj); #c1(IKZF4) c2(XP_011536971) c3(zinc finger protein Eos isoform X4) c4( ) c5(334664) c6( ) c7(Di, bEg, A, OW, qj); #c1(IKZF4) c2(XP_011536972) c3(zinc finger protein Eos isoform X5) c4( ) c5(334665) c6( ) c7(Qi, bEg, A, OW, qj); #c1(IL10) c2(XP_011507808) c3(interleukin-10 isoform X1) c4(297856) c5(297857, 297858, 297859, 297860, 297861, 297862, 297863, 297864, 297865, 297866, 297867, 297868, 297869, 297870, 297871, 297872, 297873, 297874, 297875, 297876, 297877, 297878, 297879, 297880, 297881, 297882, 297883, 297884, 297885, 297886, 297887, 297888, 297889, 297890, 297891, 297892, 297893, 297894, 297895, 297896, 297897, 297898, 297899, 297900, 297901, 297902, 297903, 297904, 297905, 297906, 297907, 297908, 297909, 297910, 297911, 297912, 297913, 297914, 297915, 297916, 297917, 297918, 297919, 297920, 297921, 297922, 297923, 297924, 297925, 297926, 297927, 297928, 297929, 297930, 297931, 297932, 297933, 297934, 297935, 297936, 297937, 297938, 297939, 297940, 297941, 297942, 297943, 297944, 297945, 297946, 297947, 297948, 297949, 297950, 297951) c6(297952) c7(T, ea, bO, fd, aoj, ci, aY, xy, aE, rw, dV, Ds, Bi, hu, hH, cp, aa, VF, ly, az, nY, vs, zC, mK, axG, ba, fT, gt, Jr, nB, dc, iA, Ji, cj, Zn, Dt, k, eg, fy, CE, aEw, LT, aYZ, bPN, ht, aj, ww, bPL, qW, cd, br, zo, aSa, lG, ey, brT, mR, aJ, sp, EU, bPr, ge, fQ, fe, CQ, adz, aBs, bOj, E, apE, DM, jv, DD, aA, cz, ol, bX, wy, q, aRo, aEE, akU, cy, j, lM, dl, eu, bA, aFS, bf, fO, lC, bg, iy, lo, Vk, pb, Cv, sk, cT, pa, iM, tW, ac, ig, iQ, Nk, tz, aan, bt, Ze, ao, gV, iW, axd, jh, dG, fu, aue, ad, wD, aoH, hX, yt, Ae, Ot, oW, lQ, wm, bPM, WZ, bPv, qZ, ff, Of, aLI, bNZ, EZ, avG, Sm, iV, bz, aYt, DF, aDH, iq, anY, aDB, ME, lf, ZW, kd, ne, rk, iu, oO, bPB, bM, qo, lD, if, e, bB, cL, gA, bh, ajC, Tz, aZO, x, bPE, bPx, ws, fS, Gb, ja, azm, bc, akl, up, JI, fc, hl, zD, kt, pD, aFU, eE, bw, eY, xD, ki, mx, aiM, hh, pJ, abh, bBT, avY, Ht, dD, wu, yG, n, aCZ, Mo, jz, bae, ec, aMj, eV, hn, bmo, xP, A, fJ, QM, GA, bds, po, amo, xe, abV, cA, Pa, PI, aOL, at, bPs, ut, eo, fL, B, vQ, pF, dA, wL, eX, YN, fF, GC, vv, bcE, v, Qo, V, aqN, DK, rS, sl, ld, bPF, XZ, ae, blm, hM, zH, vR, ev, acB, t, vk, ck, hO, yH, vi, MH, OR, wV, eq, adg, qi, Pm, hd, od, afm, Db, alz, ds, qh, bPA, hD, rN, JK, XI, sj, mu, bPm, d, je, wc, NH, aFt, dN, anW, Z, aDN, bF, D, gk, Da, Ny, wz, rH, agl, ajt, y, kS, hv, pC, fR, atj, al, CH, bPw, lj, qj, lR, wa, AW, xE, Bc, bW, bJK, M, rB, K, aZS, aDZ, DY, dF, eL, Us, bNG, bzH, mH, aCM, bPl, bn, aAT, axu, asD, mQ, LZ, hN, aB, g, ve, uC, bPH, xK, aDs, JA, nF, aer, bPC, U, uo, dW, m, asu, aZN, NK, oO, aFQ, sQ, sw, aw, fN, BH, BF, aBl, pM, zP, eh, fK, kH, cU, Ne, wb, aDp, Ni, Og, agF, nN, jT, cM, Zj, bMc, kT, ar, acC, W, SE, mg, hs, gg, lB, Pr, it, L, aGC, fM, fG, jx, wo, aSE, bJw, j, sl, aYW, pd, eA, J, o, hb, bNO, nX, jZ, bq, kl, xR, bPt, Y, aZs, Pg, apk, r, wl, aHU, en, iK, Ym, bN, sn, c, Dy, and, hc, of, dh, wR, Da, rK, dy, EY, fD, sY, am, aFe, bxK, azW, CS, LX, em, bs, bPD, auq, bS, atK, ase, Ho, dP, bPz, acu, bPG, aNp, bNL, ji, NM, qE, aVZ, tb, anX, vj, xs, Sn, cr, gE, si, hp, Bu, bNW, Nq, TA, Bv, bPn, aFb, sG, aEs, aCl, ei, hE, G, eb, Jx, mF, aR, bHS, dY, iX, pk, azx, eN, hm, DA, ayT, zQ, aAu, aW, Rr, aOW, eM, uJ, zv, aSs, qR, ajm, aEj, My, bzb, Wb, Mi, ct, bd, qN, fr, aHj, aPb, ai, eW, Vs, xC, cc, bPp, ju, h, bdp, aPe, dL, bam, Nr, asp, oo, CM, aq, mo, Ca, kk, acD, lx, oU, Wt, asE, Qy, aCz, hk, FB, bm, uz, aDE, zB, bL, Ob, ka, Dw, rG, bll, eR, yk, cR, hy, OY, C, ze, aoC, bPy, ady, ga, aCY, blC, mP, aoA, qp, afQ, aT, Tj, an, af, aZA, Uf, s, iU, oV, akY, aDm, f, OC, lY, ajl, sS, nM, Zg, alt, vZ, AH, fP, aYx, dT, CZ, ij, Kd, qX, bPo, Ke, ZI, aD, vr, jE, pz, mC, dZ, kL, LH, qC, av, bPu, F, oz, Os, vw, jo, aFl, vO, yF, uQ, bJM, bPq, yx, azP, eZ, yy, gl, aFr, xv, aF, bPK, eK, LB, es, eU, axs, atM, bE, Rv, z, aS, aGg, bPJ, ra, GF, fh, X); #c1(IL10RA) c2(NP_001549) c3(interleukin-10 receptor subunit alpha precursor) c4(118771, 118772, 118773, 118774) c5(118775, 118776, 118777, 118778, 118779, 118780, 118781, 118782, 118783, 118784, 118785, 118786, 118787, 118788, 118789, 118790, 118791, 118792, 118793, 118794, 118795, 118796, 118797, 118798) c6( ) c7(bM, sY, Dw, fM, fR, sQ, iW, al, jx, jh, fd, n, v, fr, C, uE, ai, hX, av, AW, F, bPO, dY, dV, h, Ds, dL, JK, lQ, ey, ji, eL, Uf, mR, vj, mQ, eZ, aFr, fe, lo, hp, iV, uC, adz, vk, gk, iQ, uo, at, ut, aS, fy, y, cn); #c1(IL10RB) c2(NP_000619) c3(interleukin-10 receptor subunit beta precursor) c4(13986, 13987) c5(13988, 13989, 13990, 13991, 13992, 13993, 13994, 13995, 13996, 13997, 13998, 13999) c6( ) c7(bM, Dw, fM, fR, sQ, jx, bNE, fd, pl, jE, hX, AW, h, Ds, bPP, ji, IQ, lM, eu, vj, hl, eZ, aFr, fe, lo, CQ, iV, f, RB, wz, uo, ut, y, B); #c1(IL11) c2(NP_000632) c3(interleukin-11 isoform 1 precursor) c4(14131) c5(14132, 14133, 14134, 14135, 14136, 14137, 14138, 14139) c6(14140, 14141) c7(bM, T, dD, Dw, aUI, ea, lH, aw, aA, fd, aj, eq, ci, uE, JW, ai, J, ad, dP, bPE, aE, afm, aT, cy, Ds, qG, DU, iY, ji, ae, bPQ, af, hm, qk, yo, Nr, aW, Y, Hz, hl, Pg, eH, fe, aq, lo, aF, po, g, iV, eF, t, HI, pm, zV, Nq, ck, LB, aDu, iT, aCT, hk, AM, aer, AH, gX, cj, at, uz, m, EY, HE, B, y); #c1(IL11RA) c2(NP_001136256) c3(interleukin-1l receptor subunit alpha precursor) c4(54700, 54701, 54702, 54703, 54704) c5(54705, 54706, 54707, 54708, 54709, 54710, 54711, 54712, 54713, 54714, 54715, 54716, 54717, 54718, 54719, 54720, 54721, 54722, 54723, 54724, 54725, 54726, 54727, 54728, 54729, 54730, 54731, 54732, 54733, 54734, 54735, 54736, 54737, 54738, 54739, 54740, 54741, 54742) c6( ) c7(bPR, T, fM, Ny, eX, aT, cj, eV, fd, ey, ci, m, aGg, g, t, ae, ai, ad); #c1(IL1ZA) c2(NP_000873) c3(interleukin-12 subunit alpha precursor) c4(16694, 16695, 16696, 16697, 16698, 16699) c5(16700, 16701, 16702, 16703, 16704, 16705, 16706, 16707, 16708, 16709, 16710, 16711, 16712, 16713, 16714, 16715, 16716, 16717, 16718, 16719, 16720, 16721, 16722, 16723) c6(16724, 16725, 16726, 16727, 16728, 16729) c7(bM, T, if, fd, cL, ci, aR, vV, bW, pX, dV, Ds, wt, bc, axu, g, as, eE, eF, uC, cm, fT, WO, xD, U, cj, k, dD, fN, aj, qN, eh, ai, aPb, h, lG, eV, dL, A, fe, aq, yq, Wt, kT, at, uz, eo, B, ek, Dw, fG, jx, eX, aA, DY, V, eA, aEE, mP, hb, ld, aT, qG, ae, af, hM, Y, Pg, pb, ov, sn, ac, ig, WP, sY, hl, aD, iW, jh, eq, jE, ad, wD, hX, oW, hD, uQ, eH, eZ, aF, hp, bz, aCl, xg); #c1(IL12B) c2(NP_002178) c3(interleukin-12 subunit beta precursor) c4(126331) c5(126332, 126333, 126334, 126335, 126336, 126337) c6( ) c7(bM, T, fR, Js, atj, if, fd, gA, Tz, AW, vV, dV, Ds, hu, hH, bc, aa, axu, az, LZ, da, g, eE, uC, fT, Jr, xD, k, eg, dD, sw, aw, ht, n, qN, pM, eh, ai, cc, ju, cU, h, dL, bOp, A, mR, aXy, sp, fe, aq, CQ, gG, dB, at, fL, Pr, B, Jo, cn, bL, Dw, lk, fG, jx, aA, cz, aSE, OY, j, C, Qo, V, pd, q, eA, ga, aEE, mP, jZ, apJ, aT, bPT, le, hM, aFK, r, lo, en, ov, iQ, ig, alt, dy, sY, azW, em, axd, iW, jh, dG, ah, eq, pl, kL, hX, wD, F, oW, hD, rN, yF, uQ, bPS, qZ, ff, cr, eZ, hp, iV, atM, Om, z, Ny, aED, hE, y); #c1(IL12RB1) c2(NP_001276953) c3(interleukin-12 receptor subunit beta-1 isoform 4) c4( ) c5(108714, 108715) c6( ) c7(bM, sY, bOi, Dw, lk, fR, iW, ht, jx, fd, C, wD, dP, AW, oW, dV, aDa, Ds, bOp, hu, bc, eu, uQ, aa, aEV, A, B, qZ, sp, vj, Zf, wc, cr, eZ, apx, eE, uC, bPK, op, xK, Pa, ig, bbx, cj, CZ, k, fL, y, bhp); #c1(IL12RB1) c2(NP_005526) c3(interleukin-12 receptor subunit beta-1 isoform 1 precursor) c4(157094) c5(157095, 157096, 157097, 157098, 157099, 157100, 157101, 157102) c6( ) c7(bM, sY, bOi, Dw, lk, fR, iW, ht, jx, fd, C, wD, dP, AW, oW, dV, aDa, Ds, bOp, hu, bc, eu, uQ, aa, aEV, A, B, qZ, sp, vj, Zf, wc, cr, eZ, apx, eE, uC, bPK, op, xK, Pa, ig, bbx, cj, CZ, k, fL, y, bhp); #c1(IL12RB1) c2(NP_714912) c3(interleukin-12 receptor subunit beta-1 isoform 2 precursor) c4(246596) c5(246597, 246598, 246599, 246600, 246601) c6( ) c7(bM, sY, bDi, Dw, lk, fR, iW, ht, jx, fd, C, wD, dP, AW, oW, dV, aDa, Ds, bOp, hu, bc, eu, uQ, aa, aEV, A, B, qZ, sp, vj, Zf, wc, cr, eZ, apx, eE, uC, bPK, op, xK, Pa, ig, bbx, cj, CZ, k, fL, y, bhp); #c1(IL12RB1) c2(XP_011526279) c3(interleukin-12 receptor subunit beta-1 isoform X13) c4( ) c5(320546) c6( ) c7(bM, sY, bOi, Dw, lk, fR, iW, ht, jx, fd, C, wD, dP, AW, oW, dV, aDa, Ds, bOp, hu, bc, eu, uQ, aa, aEV, A, B, qZ, sp, vj, Zf, wc, cr, eZ, apx, eE, uC, bPK, op, xK, Pa, ig, bbx, cj, CZ, k, fL, y, bhp); #c1(IL13) c2(NP_002179) c3(interleukin-13 precursor) c4(126338) c5(126339, 126340, 126341, 126342, 126343, 126344) c6( ) c7(ID, bM, G, T, aC, xS, fR, atj, if, fd, cL, zh, ci, bh, lR, Jx, asr, fw, bPX, Tz, ip, wa, AW, xE, aE, fS, dV, Ds, aDZ, azx, bc, bzH, aa, ly, bbC, bn, aAg, aW, mQ, Du, bPU, nY, kt, aB, g, eE, bJy, bwe, uC, fT, lt, buT, Jr, aCT, nB, lp, iA, aDs, cj, uo, Hb, m, eg, hh, aSs, qR, uL, abh, bFn, bBT, cE, aZt, Ht, FW, dD, akc, sQ, NT, fN, n, BH, aj, fr, qN, pM, eh, ai, acy, baA, jz, aow, kH, cU, lG, h, dL, du, ey, eV, aDp, Nr, asp, mR, A, sp, aCx, fe, po, kp, CQ, gG, kk, cM, Ix, yR, ar, aDu, dB, ajd, JM, at, uz, ut, L, bPV, bL, Dw, jv, fM, dA, DD, fG, eX, aA, cz, hz, hy, j, C, q, J, o, eA, mP, bIN, jZ, sl, apJ, aT, bPW, aAD, DX, lM, ae, af, aAi, jf, Uf, xR, oV, Y, fO, r, Ox, akY, lo, Xk, bu, en, t, ik, pb, bN, ajl, cT, Ta, bJk, DW, c, rR, iM, hr, wR, dy, yH, aan, fD, fl, blu, On, aCA, wi, aDy, iW, jh, bS, ah, eq, Fc, ase, ad, wD, aoH, dP, hX, av, hd, afm, oz, ds, Tx, hD, JK, aNp, lQ, ji, amY, XI, NM, yF, uQ, DI, d, je, bPY, ff, cr, eZ, dN, aFr, xv, aF, aDP, iV, jg, bz, DF, Nq, LB, Cq, eU, z, asq, aDB, aCl, rH, od, lC, xg, y, Tc); #c1(IL13RA1) c2(NP_001551) c3(interleukin-13 receptor subunit alpha-1 precursor) c4(118799, 118800) c5(118801, 118802, 118803, 118804, 118805, 118806, 118807, 118808, 118809, 118810, 118811, 118812) c6( ) c7(bM, T, AW, Dw, U, cr, at, Ds, JK, ey, K, eA, bu, eE, ji, pb); #c1(IL13RA1) c2(XP_011529639) c3(interleukin-13 receptor subunit alpha-isoform X2) c4(325286) c5(325287, 325288, 325289, 325290, 325291, 325292) c6( ) c7(bM, T, AW, Dw, U, cr, at, Ds, JK, ey, K, eA, bu, eE, ji, pb); #c1(IL13RA2) c2(NP_000631) c3(interleukin-13 receptor subunit alpha-2 precursor) c4(14117) c5(14118, 14119, 14120, 14121, 14122, 14123, 14124, 14125, 14126, 14127, 14128, 14129, 14130) c6( ) c7(bM, sY, T, Dw, hy, fr, ci, eA, ad, ib, aE, aT, Ds, JK, ji, amY, ae, d, oV, fc, cr, as, bu, eE, t, uC, iK, pb, DF, dB, at, m, hS); #c1(IL15) c2(NP_000576) c3(interleukin-15 isoform 1 preproprotein) c4(12844, 12845) c5(12846, 12847, 12848, 12849, 12850, 12851, 12852, 12853, 12854, 12855, 12856, 12857, 12858, 12859, 12860, 12861, 12862, 12863, 12864, 12865, 12866, 12867, 12868, 12869, 12870, 12871, 12872, 12873, 12874, 12875, 12876, 12877, 12878, 12879, 12880, 12881, 12882, 12883, 12884, 12885, 12886, 12887, 12888, 12889, 12890, 12891, 12892, 12893, 12894, 12895, 12896, 12897, 12898, 12899, 12900, 12901, 12902, 12903, 12904, 12905, 12906, 12907, 12908, 12909, 12910, 12911, 12912, 12913, 12914, 12915, 12916, 12917, 12918, 12919, 12920, 12921, 12922, 12923, 12924, 12925, 12926, 12927, 12928, 12929, 12930, 12931, 12932, 12933, 12934, 12935, 12936, 12937, 12938, 12939, 12940, 12941, 12942, 12943, 12944, 12945, 12946, 12947, 12948, 12949) c6(12950) c7(bM, T, fR, ea, e, eb, bB, fd, ci, Jx, amW, x, bv, AW, aE, Ds, rB, K, hH, cp, aa, mH, eG, aW, eE, eF, uC, bwe, cm, ou, eM, Jr, Jp, cj, uo, dW, k, m, Ht, dD, akc, sQ, NT, fr, qW, ai, eW, ft, cc, WB, ju, kH, cU, Ne, h, dL, bl, afu, Nr, A, RT, sp, CM, fe, aq, po, kk, xe, cM, adz, ar, at, pg, ut, aiS, fL, B, bL, Dw, lk, dA, DD, cz, hz, v, j, V, o, aT, ae, bA, azU, xR, oV, Y, ev, bg, lo, Ph, en, t, f, FT, bN, ck, iM, kP, bdP, Da, rk, yH, bt, jh, eq, mC, ad, wD, dP, F, fX, oW, JK, ji, lQ, iL, yF, uQ, DI, je, yx, eH, ff, cr, eZ, dN, Mz, hp, jg, iV, bz, aJx, Nq, TA, bPZ, Bv, bE, aFb, gZ, ei, y, X); #c1(IL15) c2(NP_751915) c3(interleukin-15 isoform 2 preproprotein) c4(248544) c5(248545, 248546, 248547, 248548, 248549, 248550, 248551, 248552, 248553, 248554, 248555, 248556, 248557, 248558) c6( ) c7(bM, T, fR, ea, e, eb, bB, fd, ci, Jx, amW, x, bv, AW, aE, Ds, rB, K, hH, cp, aa, mH, eG, aW, eE, eF, uC, bwe, cm, ou, eM, Jr, Jp, cj, uo, dW, k, m, Ht, dD, akc, sQ, NT, fr, qW, ai, eW, ft, cc, WB, ju, kH, cU, Ne, h, dL, bl, afu, Nr, A, RT, sp, CM, fe, aq, po, kk, xe, cM, adz, ar, at, pg, ut, aiS, fL, B, bL, Dw, lk, dA, DD, cz, hz, v, j, V, o, aT, ae, bA, azU, xR, oV, Y, ev, bg, lo, Ph, en, t, f, FT, bN, ck, iM, kP, bdP, Da, rK, yH, bt, jh, eq, mC, ad, wD, dP, F, fX, oW, JK, ji, lQ, iL, yF, uQ, DI, je, yx, eH, ff, cr, eZ, dN, Mz, hp, jg, iV, bz, alx, Nq, TA, bPZ, Bv, bE, afb, gZ, ei, y, X); #c1(IL15RA) c2(NP_001230468) c3(interleukin-15 receptor subunit alpha isoform 3) c4(86311, 86312) c5(86313, 86314, 86315, 86316, 86317, 86318, 86319, 86320, 86321, 86322, 86323) c6( ) c7(bM, Dw, fM, akc, v, j, ad, F, nX, kH, aT, Ds, dL, lQ, ae, Nr, aW, vj, cr, dN, po, fe, beP, g, iV, bJ, Nq, ck, bE, at, ei, ne); #c1(IL15RA) c2(NP_002180) c3(interleukin-15 receptor subunit alpha isoform 1 precursor) c4(126345, 126346) c5(126347, 126348, 126349, 126350, 126351, 126352, 126353) c6( ) c7(bM, Dw, fM, akc, v, j, ad, F, nX, kH, aT, Ds, dL, lQ, ae, Nr, aW, vj, cr, dN, po, fe, beP, g, iV, bJ, Nq, ck, bE, at, ei, ne); #c1(IL15RA) c2(NP_751950) c3(interleukin-15 receptor subunit alpha isoform 2 precursor) c4(248603, 248604) c5(248605, 248606, 248607, 248608, 248609, 248610) c6( ) c7(bM, Dw, fM, akc, v, j, ad, F, nX, kH, aT, Ds, dL, lQ, ae, Nr, aW, vj, cr, dN, po, fe, beP, g, iV, bJ, Nq, ck, bE, at, ei, ne); #c1(IL15RA) c2(XP_011517768) c3(interleukin-15 receptor subunit alpha isoform X6) c4( ) c5(311630, 311631, 311632, 311633, 311634, 311635, 311636, 311637, 311638, 311639, 311640, 311641, 311642, 311643, 311644, 311645, 311646, 311647) c6( ) c7(bM, Dw, fM, akc, v, j, ad, F, nX, KH, aT, Ds, dL, lQ, ae, Nr, aW, vj, cr, dN, po, fe, beP, g, iV, bJ, Nq, ck, bE, at, ei, ne); #c1(IL15RA) c2(XP_011517769) c3(interleukin-15 receptor subunit alpha isoform X7) c4( ) c5(311648) c6( ) c7(bM, Dw, fM, akc, v, j, ad, F, nX, kH, aT, Ds, dL, lQ, ae, Nr, aW, vj, cr, dN, po, fe, beP, g, iV, bJ, Nq, ck, bE, at, ei, ne); #c1(IL15RA) c2(XP_011517770) c3(interleukin-15 receptor subunit alpha isoform X8) c4(311649) c5(311650, 311651, 311652, 311653, 311654) c6( ) c7(bM, Dw, fM, akc, v, j, ad, F, nX, KH, aT, Ds, dL, lQ, ae, Nr, aW, vj, cr, dN, po, fe, beP, g, iV, bJ, Nq, ck, bE, at, ei, ne); #c1(IL15RA) c2(XP_011517774) c3(interleukin-15 receptor subunit alpha isoform X12) c4(311655) c5( ) c6( ) c7(bM, Dw, fM, akc, v, j, ad, F, nX, kH, aT, Ds, dL, lQ, ae, Nr, aW, vj, cr, dN, po, fe, beP, g, iV, bJ, Nq, ck, bE, at, ei, ne); #c1(IL15RA) c2(XP_011517778) c3(interleukin-15 receptor subunit alpha isoform X16) c4(311656) c5(311657, 311658, 311659, 311660, 311661, 311662) c6( ) c7(bM, Dw, fM, akc, v, j, ad, F, nX, kH, aT, Ds, dL, lQ, ae, Nr, aW, vj, cr, dN, po, fe, beP, g, iV, bJ, Nq, ck, bE, at, ei, ne); #c1(IL15RA) c2(XP_011517779) c3(interleukin-15 receptor subunit alpha isoform X17) c4(311663) c5(311664, 311665, 311666, 311667, 311668, 311669) c6( ) c7(bM, Dw, fM, akc, v, j, ad, F, nX, kH, aT, Ds, dL, lQ, ae, Nr, aW, vj, cr, dN, po, fe, beP, g, iV, bJ, Nq, ck, bE, at, ei, ne); #c1(IL16) c2(NP_001165599) c3(pro-interleukin-16 isoform 3) c4(67816, 67817, 67818, 67819, 67820) c5(67821, 67822, 67823, 67824, 67825, 67826, 67827, 67828, 67829, 67830, 67831, 67832, 67833, 67834, 67835, 67836, 67837, 67838, 67839, 67840, 67841, 67842, 67843, 67844, 67845, 67846, 67847, 67848) c6( ) c7(bM, T, Dw, FR, if, iW, jx, jh, aA, cz, n, fd, j, jE, qN, lR, fr, pz, q, cd, ad, hX, bOV, dP, x, Db, sN, apJ, aT, hD, SH, Ds, hn, h, ji, ae, aa, d, je, xR, aW, oV, fJ, bg, gC, eZ, r, dN, nY, fe, aF, iV, eE, t, uC, pb, ck, es, cT, ok, iM, xT, bQa, xO, z, cj, at, uz, k, m, od, hh, y, B); #c1(IL16) c2(XP_005254399) c3(pro-interleukin-16 isoform X3) c4(273181) c5(273182, 273183, 273184) c6( ) c7(bM, T, Dw, fR, if, iW, jx, jh, aA, cz, n, fd, j, jE, qN, lR, fr, pz, q, cd, ad, hX, bOV, dP, x, Db, sN, apJ, aT, HD, SH, Ds, hn, h, ji, ae, aa, d, je, xR, aW, oV, fJ, bg, gC, eZ, r, dN, nY, fe, aF, iV, BE, t, uC, pb, ck, es, cT, ok, iM, xT, bQa, xO, z, cj, at, uz, k, m, od, hh, y, B); #c1(IL16) c2(XP_005254403) c3(pro-interleukin-16 isoform XI) c4(273185) c5(273186, 273187, 273188, 273189, 273190, 273191, 273192, 273193, 273194, 273195, 273196) c6( ) c7(bM, T, Dw, fR, if, iW, jx, jh, aA, cz, n, fd, j, jE, qN, lR, fr, pz, q, cd, ad, hX, bOV, dP, x, Db, sN, apJ, aT, HD, SH, Ds, hn, h, ji, ae, aa, d, je, xR, aW, oV, fJ, bg, gC, eZ, r, dN, nY, fe, aF, iV, eE, t, uC, pb, ck, es, cT, ok, iM, xT, bQa, xO, z, cj, at, uz, k, m, od, hh, y, B); #c1(IL16) c2(XP_011519820) c3(pro-interleukin-16 isoform X4) c4(314560) c5( ) c6( ) c7(bM, T, Dw, FR, if, iW, jx, jh, aA, cz, n, fd, j, jE, qN, lR, fr, pz, q, cd, ad, hX, bOV, dP, x, Db, sN, apJ, aT, hD, SH, Ds, hn, h, ji, ae, aa, d, je, xR, aW, oV, fJ, bg, gC, eZ, r, dN, nY, fe, aF, iV, eE, t, uC, pb, ck, es, cT, ok, iM, xT, bQa, xO, z, cj, at, uz, k, m, od, hh, y, B); #c1(IL16) c2(XP_011519821) c3(pro-interleukin-16 isoform X5) c4( ) c5(314561, 314562, 314563, 314564, 314565, 314566) c6( ) c7(bM, T, Dw, FR, if, iW, jx, jh, aA, cz, n, fd, j, jE, qN, lR, fr, pz, q, cd, ad, hX, bOV, dP, x, Db, sN, apJ, aT, HD, SH, Ds, hn, h, ji, ae, aa, d, je, xR, aW, oV, fJ, bg, gC, eZ, r, dN, nY, fe, aF, iV, eE, t, uC, pb, ck, es, cT, ok, iM, xT, bQa, xO, z, cj, at, uz, k, m, oQ, hh, y, B); #c1(IL16) c2(XP_011519822) c3(pro-interleukin-16 isoform X2) c4(314567) c5(314568, 314569, 314570, 314571, 314572, 314573, 314574) c6( ) c7(bM, T, Dw, FR, if, iW, jx, jh, aA, cz, n, fd, j, jE, qN, lR, fr, pz, q, cd, ad, hX, bOV, dP, x, Db, sN, apJ, aT, HD, SH, Ds, hn, h, ji, ae, aa, d, je, xR, aW, oV, fJ, bg, gC, eZ, r, dN, nY, fe, aF, iV, eE, t, uC, pb, ck, es, cT, ok, iM, xT, bQa, xO, z, cj, at, uz, k, m, od, hh, y, B); #c1(IL17A) c2(NP_002181) c3(interleukin-17A precursor) c4(126354) c5(126355, 126356, 126357, 126358, 126359, 126360) c6( ) c7(oO, bM, T, fR, ea, if, e, al, fd, bB, cL, Vd, zh, ci, qj, Op, aY, aDI, wa, xE, aDk, Bc, bW, dE, M, Ds, Bi, K, aDZ, hu, hH, LG, dF, bc, eL, hm, tw, DA, JI, aW, az, hl, aXc, asD, mQ, HA, fc, pL, nr, eE, eF, mK, aik, ba, fT, lt, y, eM, Jr, xD, aCT, aDs, mx, U, cj, m, gn, fy, aSs, uL, Ht, dD, aw, yG, ht, fN, aj, uE, pM, qN, pU, qW, cd, ai, eW, baA, ke, zw, pN, Xe, cU, h, eV, dL, ZN, aDp, bxg, hi, Nr, mR, A, EU, arp, blc, po, fe, Zb, Wt, aBs, aDu, Pa, bbx, lf, JM, bm, at, uz, eo, fL, Pr, B, Dw, lk, eR, DD, aYB, fG, lH, eX, aA, cz, hz, v, j, ON, C, OY, V, pd, q, J, eA, ady, aCX, bNO, jZ, aCV, aT, Di, cy, qG, j, bq, Cy, dl, eu, le, qw, bNR, bf, s, xR, Y, fO, Pg, bg, la, r, aMA, lo, aDm, en, ajF, t, pb, cT, iQ, ua, c, ac, ig, wO, hc, dh, AH, dT, wR, CZ, yH, fD, byK, zu, ao, vi, hl, akd, Ke, aDy, aD, dM, df, bQb, ha, jh, wV, eq, jE, atK, mC, wD, LH, dP, hX, av, sN, oz, ds, oW, Os, dm, HD, JK, vB, lQ, ji, NM, yF, uQ, DI, d, lx, Zf, ff, eH, cr, eZ, CF, dN, aF, hp, iV, bz, aDH, Nq, gk, Bv, eU, zr, bE, z, aCl, gy, aS, bPJ, cP, ME, hE, ra, iu, bEt, pC); #c1(IL17B) c2(NP_055258) c3(interleukin-178 precursor) c4(180101) c5(180102, 180103, 180104, 180105, 180106, 180107) c6( ) c7(bM, T, dD, ea, lH, qj, eq, mF, ai, xE, aE, ws, M, aT, cU, cy, K, lQ, bU, af, bA, aW, fO, cr, eZ, hp, iV, eE, bz, fq, DF, wg, ac, eM, bbx, Dt, byK, y, pC); #c1(IL17C) c2(NP_037410) c3(interleukin-17C precursor) c4(175360) c5(175361, 175362, 175363, 175364, 175365, 175366) c6( ) c7(T, hl, fM, dA, aNk, fN, aA, bB, fd, aj, jh, jE, V, mC, J, ai, hX, eA, xE, aT, cU, ahr, h, aNj, bK, ji, Qe, A, d, aW, Y, yx, ev, hl, cr, r, dN, eZ, lo, aF, hp, fe, jg, iV, kk, t, eE, cm, Wt, ek, eU, ac, bE, cj, at, MJ, eo, m, fL, y); #c1(IL17D) c2(NP_612141) c3(interleukin-17D precursor) c4(236041) c5(236042, 236043, 236044, 236045, 236046) c6( ) c7(oO, bM, T, ea, al, bB, zh, xE, ws, K, hu, DY, hH, DA, pL, eE, axp, ba, lt, eM, dW, m, qR, cZ, dD, JZ, ht, aj, qW, ai, aDF, h, bQd, dd, A, mR, fe, jT, ar, W, bbx, at, eo, Pr, B, bL, jx, fF, v, ON, C, V, pd, q, J, DK, cy, Cy, ae, oV, Y, fO, bg, r, aMA, lo, aDm, ck, ig, AH, dT, wR, CZ, yH, fD, byK, bt, Ke, jh, eq, jE, atK, ad, hX, oz, ds, Os, hD, ji, lQ, Zf, cr, eZ, dN, iV, bz, bE, z, agl, hE, y); #c1(IL17D) c2(XP_011533418) c3(interleukin-17D isoform X1) c4( ) c5(329514) c6( ) c7(oO, bM, T, ea, al, bB, zh, xE, ws, K, hu, DY, hH, DA, pL, eE, axp, ba, lt, eM, dW, m, qR, cZ, dD, JZ, ht, aj, qW, ai, aDF, h, bQd, dd, A, mR, fe, jT, ar, W, bbx, at, eo, Pr, B, bL, jx, fF, v, ON, C, V, pd, q, J, DK, cy, Cy, ae, oV, Y, f, bg, r, aMA, lo, aDm, ck, ig, AH, dT, wR, CZ, yH, fD, byK, bt, Ke, jh, eq, jE, atK, ad, hX, oz, ds, Os, hD, ji, lQ, Zf, cr, eZ, dN, iV, bz, bE, z, agl, hE, y); #c1(IL17F) c2(XP_011512578) c3(interleukin-(7F isoform X1) c4(303488) c5(303489, 303490, 303491, 303492, 303493, 303494) c6( ) c7(bM, T, aC, akd, aw, aA, bB, aj, wy, pd, J, xE, aE, ds, aT, hD, dL, ji, hH, eL, aa, qw, Nr, JI, aW, cr, eZ, bQc, po, fe, af, iV, eE, bz, pb, mk, Nq, bQe, fT, lt, Pa, bE, at, dT, CZ, y, bt); #c1(IL17RA) c2(NP_001276834) c3(interleukin-17 receptor A isoform 2 precursor) c4(108387) c5(108388, 108389, 108390, 108391, 108392, 108393) c6( ) c7(gh, bM, Dw, eR, cz, aY, atK, ad, wD, sN, ds, Ds, ae, eu, eH, lo, eF, Pa, bOf, wR, CZ, yH, y); #c1(IL17RA) c2(NP_055154) c3(interleukin-17 receptor A isoform 1 precursor) c4(179484) c5(179485, 179486, 179487, 179488, 179489, 179490) c6( ) c7(gh, bM, Dw, eR, cz, aY, atK, ad, wD, sN, ds, Ds, ae, eu, eH, lo, eF, Pa, bQf, wR, CZ, yH, y); #c1(IL17RB) c2(NP_061195) c3(interleukin-17 receptor B precursor) c4(199917) c5(199918, 199919, 199920, 199921, 199922, 199923) c6( ) c7(bM, T, A, xE, iT, aW, aT, fd, n, aoj, m, t); #c1(IL17RC) c2(NP_001190192) c3(interleukin-17 receptor C isoform 4 precursor) c4(80339) c5(80340, 80341, 80342, 80343, 80344, 80345) c6( ) c7(gh, T, W, jZ, wR, V, ae, ad); #c1(IL17RC) c2(NP_001190193) c3(interleukin-17 receptor C isoform 5 precursor) c4( ) c5(80346) c6( ) c7(gh, T, W, jZ, wR, V, ae, ad); #c1(IL17RC) c2(NP_001190194) c3(interleukin-17 receptor C isoform 6 precursor) c4(80347, 80348) c5(80349, 80350, 80351, 80352, 80353, 80354, 80355, 80356, 80357, 80358, 80359, 80360, 80361, 80362, 80363, 80364, 80365) c6( ) c7(gh, T, W, jZ, wR, V, ae, ad); #c1(IL17RC) c2(NP_116121) c3(interleukin-17 receptor C isoform 3 precursor) c4(225428, 225429) c5(225430, 225431, 225432, 225433, 225434, 225435) c6( ) c7(gh, T, W, jZ, wR, V, ae, ad); #c1(IL17RC) c2(NP_703190) c3(interleukin-17 receptor C isoform 1 precursor) c4(246083) c5(246084, 246085, 246086, 246087, 246088, 246089) c6( ) c7(gh, T, W, jZ, wR, V, ae, ad); #c1(IL17RC) c2(NP_703191) c3(interleukin-17 receptor C isoform 2 precursor) c4(246090) c5(246091, 246092, 246093, 246094, 246095, 246096) c6( ) c7(gh, T, W, jZ, wR, V, ae, ad); #c1(IL17RD) c2(NP_060033) c3(interleukin-17 receptor D precursor) c4(193781) c5(193782, 193783, 193784, 193785, 193786, 193787, 193788, 193789, 193790, 193791, 193792, 193793, 193794, 193795, 193796) c6( ) c7(T, agl, qK, oW, ci, bX, ae, ad, bQg); #c1(IL17RD) c2(XP_005265295) c3(interleukin-17 receptor D isoform X2) c4( ) c5(279462) c6( ) c7(T, agl, qK, oW, ci, bX, ae, ad, blg); #c1(IL17RD) c2(XP_006713272) c3(interleukin-17 receptor D isoform X3) c4( ) c5(287066, 287067, 287068, 287069, 287070) c6( ) c7(T, agl, qK, oW, ci, bX, ae, ad, bQg); #c1(IL17RD) c2(XP_011532151) c3(interleukin-17 receptor D isoform X1) c4(328206, 328207, 328208) c5(328209, 328210, 328211, 328212, 328213, 328214, 328215) c6( ) c7(T, agl, qK, oW, ci, bX, ae, ad, bQg); #c1(IL17RE) c2(NP_001180309) c3(interleukin-17 receptor E isoform 6 precursor) c4(73822) c5(73823, 73824, 73825, 73826, 73827, 73828, 73829, 73830) c6(73831, 73832) c7(dV, oW, eg); #c1(IL17RE) c2(NP_705613) c3(interleukin-17 receptor E isoform 1 precursor) c4( ) c5(246126, 246127, 246128, 246129, 246130, 246131, 246132, 246133, 246134, 246135, 246136, 246137) c6(246138, 246139, 246140, 246141) c7(dV, oW, eg); #c1(IL17RE) c2(NP_705614) c3(interleukin-17 receptor E isoform 2) c4( ) c5(246142, 246143, 246144, 246145, 246146, 246147, 246148, 246149) c6(246150, 246151) c7(dV, oW, eg); #c1(IL17RE) c2(NP_705616) c3(interleukin-17 receptor E isoform 5) c4(246152, 246153) c5(246154, 246155, 246156, 246157, 246158, 246159, 246160, 246161, 246162, 246163) c6(246164, 246165) c7(dV, oW, eg); #c1(IL17RE) c2(XP_006713038) c3(interleukin-17 receptor E isoform X3) c4(286985) c5( ) c6( ) c7(dV, oW, eg); #c1(IL17REL) c2(NP_001001694) c3(putative interleukin-17 receptor E-like) c4(18950) c5(18951, 18952, 18953, 18954, 18955, 18956, 18957, 18958) c6( ) c7(iV); #c1(IL17REL) c2(XP_011528991) c3(putative interleukin-17 receptor E-like isoform X1) c4( ) c5(324356, 324357) c6( ) c7(iV); #c1(IL18) c2(NP_001230140) c3(interleukin-18 isoform 2 proprotein) c4(85982) c5(85983, 85984, 85985, 85986, 85987, 85988, 85989, 85990) c6(85991, 85992) c7(oO, bM, T, UH, fR, azf, pf, vf, ea, e, if, CH, fd, cL, ci, bh, aY, Tz, amW, oQ, xy, xE, lu, Bc, bW, M, JT, dV, ja, Ds, K, bah, hu, wt, hH, pk, DY, bc, akX, cp, aa, hm, DA, aiV, up, YY, aW, az, hl, asD, md, fc, g, eE, cm, uC, GR, ba, y, fT, lt, iA, iT, lp, vA, mx, wB, cj, uo, dW, mk, Hb, m, lS, eg, cE, abh, nD, dD, JZ, sQ, lo, ht, aw, fN, n, BH, aj, fr, ct, uE, qN, qW, cd, bQh, ai, baA, cc, hw, ec, brX, aAh, Lv, cU, jy, h, eV, uV, aDp, A, CN, sp, fe, aq, nN, cM, op, ar, kT, GG, W, xO, NL, lf, JM, at, ut, eo, fL, bni, Pr, B, cB, it, bL, Ob, Dw, aAf, lk, DD, wL, fG, jx, eX, rf, aA, cz, fF, hy, v, j, C, bX, V, q, J, o, ady, aPW, ga, aEE, mP, WY, apJ, qp, rS, aT, cy, qG, aAD, rz, Cy, ae, af, dl, eu, kl, iU, oV, Y, fO, bg, r, lo, aZg, en, t, iK, pb, ck, ajl, cT, bJk, sn, ac, pZ, ig, hc, AH, Uq, gX, fP, rK, dy, EY, JH, fD, bt, ao, vi, hS, Kd, sY, Ve, bmQ, akd, Ke, ZI, MH, aZ, dM, aD, iW, jh, oR, bS, bQi, jE, ah, eq, pz, hF, mC, ad, dZ, hX, qC, dP, l, F, afm, oz, qh, oW, hD, vB, ji, lQ, jo, bQj, adx, NM, nJ, yF, uQ, cW, ff, cr, eZ, brp, aF, hp, Fq, iV, Z, bz, ek, LB, D, zr, bE, z, aDB, aED, ll, wz, gZ, aCl, rH, ei, Lz, GF, hE, hv); #c1(IL18) c2(NP_001553) c3(interleukin-18 isoform 1 proprotein) c4(118813) c5(118814, 118815, 118816, 118817, 118818, 118819, 118820, 118821, 118822, 118823, 118824, 118825, 118826, 118827, 118828, 118829, 118830, 118831, 118832, 118833, 118834, 118835, 118836, 118837, 118838, 118839, 118840, 118841, 118842, 118843, 118844, 118845, 118846, 118847, 118848, 118849, 118850, 118851, 118852, 118853, 118854, 118855, 118856, 118857, 118858, 118859, 118860, 118861, 118862, 118863, 118864, 118865, 118866, 118867, 118868, 118869, 118870, 118871, 118872, 118873, 118874, 118875, 118876, 118877, 118878, 118879, 118880, 118881, 118882, 118883, 118884, 118885, 118886, 118887, 118888, 118889, 118890, 118891, 118892, 118893, 118894, 118895, 118896, 118897, 118898, 118899, 118900, 118901, 118902, 118903, 118904, 118905, 118906, 118907, 118908, 118909, 118910, 118911, 118912, 118913) c6(118914, 118915, 118916, 118917) c7(oO, bM, T, UH, fR, azf, pf, vf, ea, e, if, CH, fd, cL, ci, bh, aY, Tz, amW, ol, xy, xE, lu, Bc, bW, M, JT, dV, ja, Ds, K, bah, hu, wt, hH, pk, DY, bc, akX, cp, aa, hm, DA, aiV, up, YY, aW, az, hl, asD, md, fc, g, eE, cm, uC, GR, ba, y, fT, lt, iA, iT, lp, vA, mx, wB, cj, uo, dW, mk, Hb, m, lS, eg, cE, abh, nD, dD, JZ, sQ, lo, ht, aw, fN, n, BH, aj, fr, ct, uE, qN, qW, cd, bQh, ai, baA, cc, hw, ec, brX, aAh, Lv, cU, jy, h, eV, uV, aDp, A, CN, sp, fe, aq, nN, cM, op, ar, kT, GG, W, xO, NL, lf, JM, at, ut, eo, fL, bni, Pr, B, cB, it, bL, Ob, Dw, aAf, Ik, DD, wL, fG, jx, eX, rf, aA, cz, fF, hy, v, j, C, bX, V, q, J, o, ady, aPW, ga, aEE, mP, WY, apJ, qp, rS, aT, cy, qG, aAD, rz, Cy, ae, af, dl, eu, kl, iU, oV, Y, fO, bg, r, lo, aZg, en, t, iK, pb, ck, ajl, cT, bJk, sn, ac, pZ, ig, hc, AH, Uq, gX, fP, rK, dy, EY, JH, fD, bt, ao, vi, hS, Kd, sY, Ve, bmQ, akd, Ke, ZI, MH, aZ, dM, aD, iW, jh, oR, bS, bQi, jE, ah, eq, pz, hF, mC, ad, dZ, hX, qC, dP, l, F, afm, oz, qh, oW, hD, vB, ji, lQ, jo, bQj, adx, NM, nJ, yF, uQ, cW, ff, cr, eZ, brp, af, hp, Fq, iV, Z, bz, ek, LB, D, zr, bE, z, aDB, aED, ll, wz, gZ, aCl, rH, ei, Lz, GF, hE, hv); #c1(IL18BP) c2(NP_001034749) c3(interleukin-18-binding protein isoform a precursor) c4(32205) c5(32206, 32207, 32208, 32209, 32210, 32211, 32212, 32213, 32214, 32215, 32216, 32217, 32218, 32219, 32220, 32221, 32222, 32223, 32224, 32225, 32226, 32227, 32228, 32229, 32230, 32231, 32232, 32233, 32234, 32235, 32236, 32237, 32238, 32239, 32240, 32241, 32242, 32243) c6(32244, 32245, 32246, 32247, 32248, 32249, 32250, 32251, 32252, 32253, 32254, 32255) c7(sY, T, dD, ea, aw, jx, aET, j, eq, zP, V, cd, ga, cy, DY, bQj, A, hl, uC, c, mx, m, hE, y); #c1(IL18BP) c2(NP_001138527) c3(interleukin-18-binding protein isoform b precursor) c4(56953) c5(56954, 56955, 56956, 56957, 56958, 56959, 56960, 56961) c6(56962, 56963) c7(sY, T, dD, ea, aw, jx, aET, j, eq, zP, V, cd, ga, cy, DY, bQj, A, hl, uC, c, mx, m, hE, y); #c1(IL18BP) c2(XP_006718473) c3(interleukin-18-binding protein isoform X2) c4(291876) c5(291877, 291878, 291879, 291880, 291881, 291882, 291883, 291884) c6(291885, 291886) c7(sY, T, dD, ea, aw, jx, aET, j, eq, zP, V, cd, ga, cy, DY, bQj, A, hl, uC, c, mx, m, hE, y); #c1(IL18BP) c2(XP_011543019) c3(interleukin-18-binding protein isoform X1) c4(341716) c5(341717, 341718, 341719, 341720, 341721, 341722, 341723, 341724) c6(341725, 341726) c7(sY, T, dD, ea, aw, jx, aET, j, eq, zP, V, cd, ga, cy, DY, bQj, A, hl, uC, c, mx, m, hE, y); #c1(IL18R1) c2(NP_003846) c3(interleukin-18 receptor 1 isoform 1 precursor) c4(141353) c5(141354, 141355, 141356, 141357, 141358, 141359, 141360, 141361, 141362, 141363, 141364, 141365, 141366, 141367) c6( ) c7(bL, bM, T, Dw, aAf, dA, fR, aEj, AL, lD, fG, jx, aA, n, v, j, pl, V, ip, hX, x, AW, aE, Bj, ds, rl, dV, aT, qU, h, Ds, dz, JK, aAD, pq, af, iL, uQ, ly, A, d, mR, aAu, aW, Y, zb, oV, vj, eZ, fe, aF, hp, CA, jg, iV, eE, iK, bz, DF, lx, ck, cT, RB, eM, iT, z, rK, k, y, bt, B, cn); #c1(IL18R1) c2(XP_005264097) c3(interleukin-18 receptor 1 isoform X2) c4(278667) c5( ) c6( ) c7(bL, bM, T, Dw, aAf, dA, fR, aEj, AL, lD, fG, jx, aA, n, v, j, pl, V, ip, hX, x, AW, aE, Bj, ds, rl, dV, aT, qU, h, Ds, dz, JK, aAD, pq, af, iL, uQ, ly, A, d, mR, aAu, aW, Y, zb, oV, vj, eZ, fe, aF, hp, CQ, jg, iV, BE, ik, bz, DF, lx, ck, cT, RB, eM, iT, z, rk, k, y, bt, B, cn); #c1(IL18R1) c2(XP_011510401) c3(interleukin-18 receptor 1 isoform X3) c4( ) c5(300960, 300961, 300962, 300963, 300964, 300965) c6( ) c7(bL, bM, T, Dw, aAf, dA, fR, aEj, AL, lD, fG, jx, aA, n, v, j, pl, V, ip, hX, x, AW, aE, Bj, ds, rl, dV, aT, qU, h, Ds, dz, JK, aAD, pq, af, iL, uQ, ly, A, d, mR, aAu, aW, Y, zb, oV, vj, eZ, fe, aF, hp, CQ, jg, lV, eE, ik, bz, DF, lx, ck, cT, RB, eM, iT, z, rk, k, y, bt, B, cn); #c1(IL18RAP) c2(NP_003844) c3(interleukin-18 receptor accessory protein precursor) c4(141336, 141337) c5(141338, 141339, 141340, 141341, 141342, 141343, 141344) c6( ) c7(bM, AW, A, pf, rl, M, cj, nf, ge, cL, fe, hp, j, iV, y, aDW); #c1(IL18RAP) c2(XP_011510390) c3(interleukin-18 receptor accessory protein isoform X1) c4(300934) c5(300935, 300936, 300937, 300938, 300939) c6( ) c7(bM, AW, A, pf, rl, M, cj, nf, ge, cL, fe, hp, j, iV, y, aDW); #c1(IL18RAP) c2(XP_011510391) c3(interleukin-18 receptor accessory protein isoform X2) c4( ) c5(300940, 300941, 300942, 300943, 300944, 300945) c6( ) c7(bM, AW, A, pf, rl, M, cj, nf, ge, cL, fe, hp, j, iV, y, aDW); #c1(IL19) c2(NP_037503) c3(interleukin-19 isoform 2 precursor) c4(175873) c5(175874, 175875, 175876, 175877, 175878) c6( ) c7(bM, Dw, atj, jx, fd, jE, C, AO, hX, aT, cU, h, Ds, ji, lQ, A, qt, aW, ff, bg, cr, eZ, en, iV, eE, zV, cT, alt, cj, Dt, byK, adg, y); #c1(IL19) c2(NP_715639) c3(interleukin-19 isoform 1) c4(246698) c5(246699, 246700, 246701, 246702, 246703, 246704, 246705) c6( ) c7(bM, Dw, atj, jx, fd, jE, C, AO, hX, aT, cU, h, Ds, ji, lQ, A, qt, aW, ff, bg, cr, eZ, en, iV, eE, zV, cT, alt, cj, Dt, byK, adg, y); #c1(IL1A) c2(NP_000566) c3(interleukin-1 alpha proprotein) c4(12738) c5(12739, 12740, 12741, 12742, 12743, 12744) c6( ) c7(T, ea, bO, fd, ci, aY, aE, rw, dV, Ds, zy, hH, azh, cp, aa, yo, az, ih, nY, zC, GR, zV, ba, fT, dc, cj, Uz, eg, cE, dt, aYZ, aj, qW, cd, zo, lG, ey, on, pl, mR, anP, sp, aRL, ge, fe, CQ, ajz, bOj, DM, aA, cz, bgR, q, zn, cy, aAD, Cy, dl, eu, bA, bf, fO, bg, iy, lo, rs, pb, Cv, ov, cT, rR, OW, ac, iM, ig, awN, iQ, aDR, bt, zA, ao, iW, rl, aZH, jh, dG, uu, ad, wD, hX, abq, oW, fn, iY, lQ, cH, ff, dq, EZ, Sm, iV, bQk, bz, Xs, dX, lf, lC, Pk, ne, rk, iu, ra, oO, bM, vS, if, e, bB, cL, bh, Ul, Tz, ban, bQs, eG, dH, fc, hl, aw, kt, pD, as, ir, nr, eE, eF, qJ, bQl, ii, AV, aiM, apy, dD, yG, n, oY, uE, brY, b, no, Hx, pe, zw, eV, awL, xP, A, fJ, lb, bQq, ax, yq, amo, abV, cA, hL, at, pg, ut, eo, FL, B, dA, wL, eX, fF, GC, afK, v, aNG, V, eJ, yw, aLS, rS, sl, ld, ae, hM, ev, acB, bu, t, aff, zJ, ck, hO, yH, vi, df, bQb, OR, eq, Pm, hd, Db, ds, LA, rN, ab, hD, JK, d, je, wc, rM, dN, Z, D, gk, akl, yE, Ny, wz, gZ, aef, y, CW, hv, aC, fR, al, Qp, aMB, xE, Bc, bQo, bW, M, K, aZS, dF, eL, bzH, HI, axu, hN, g, pL, bwe, uC, ly, rq, aDs, uy, U, adl, uo, vn, m, bi, sQ, lo, aw, aBl, abd, eh, asS, kH, cU, wb, ap, tx, alw, nN, jT, cM, amt, ar, kT, W, afc, JM, Pr, L, aAf, fM, fG, jx, aee, hz, j, il, pd, eA, J, o, MR, lg, qG, bq, bPQ, kl, Ue, Y, r, en, wA, iK, brZ, hc, dh, rk, dy, bam, fD, sY, aFe, bxK, aZ, bS, ah, dP, fU, NY, vB, ji, NM, qE, aZl, eH, Sn, cr, nf, hp, Bv, Lj, aFb, eB, ei, hE, vt, bJx, zh, H, eN, hm, ayT, aAu, aW, lh, OZ, hJ, yd, Jp, R, qR, aEj, bQp, wh, fr, ct, qN, aPb, ai, Vs, cc, qg, ql, rl, h, jy, dL, bam, aq, kp, dw, Wt, hk, uw, bm, uz, te, eQ, amb, bni, bL, Dw, bll, eR, C, ady, mj, ga, cF, mP, qp, aT, bK, an, KU, af, iU, oV, f, aDQ, ajl, nM, pZ, Zg, vZ, aGo, fP, lJ, Kd, Ke, ZI, aD, ha, lD, pz, mC, dZ, XB, F, ib, xl, Os, jo, aFl, uQ, Di, eZ, aFr, aF, bQr, ek, LB, es, zr, atM, bE, dJ, aS, aGg, Lz, pk, aAN, us); #c1(IL1B) c2(NP_000567) c3(interleukin-1 beta proprotein) c4(12745, 12746, 12747, 12748) c5(12749, 12750, 12751, 12752, 12753, 12754, 12755, 12756, 12757, 12758, 12759, 12760, 12761, 12762, 12763, 12764, 12765, 12766, 12767, 12768, 12769, 12770, 12771, 12772) c6( ) c7(T, ea, bO, fd, Vd, ci, aY, aDI, bQB, aE, rw, dV, Ds, hu, zy, hH, bQJ, azh, cp, aa, li, yo, az, ih, nY, vs, zC, lk, GR, zV, ba, fT, dc, WS, cj, k, lS, eg, cE, aEw, LT, nD, dt, aYZ, ht, aj, qW, cd, zo, lG, aNj, ey, pl, mR, anP, sp, EU, aRL, ge, fQ, fe, CQ, ajz, adz, hq, iO, RB, dB, bPc, bOj, bjx, Ei, ajd, pV, E, DM, aZy, aA, cz, yV, bgR, q, zn, yu, Di, cy, aAD, j, DX, dl, eu, bA, bf, fO, bg, iy, lo, rs, GF, pb, ov, rR, OW, ac, iM, ig, awN, iQ, bQC, mG, DW, tz, aDR, bt, zA, bQz, ao, rA, gV, wi, iW, uG, rl, jh, dG, uu, fu, Gn, xt, ad, hX, wD, abq, oW, iY, lQ, NP, at J, lx, cH, cW, ff, Of, nh, dq, brp, EZ, Sm, aFd, bQk, iV, bz, aYt, DF, aDH, amK, baa, Xs, dX, ME, Pk, lC, lf, ne, rk, iu, bsJ, oO, bM, ra, bQl, if, e, bB, cL, bh, Tz, x, bQD, aZn, azm, ben, bc, bs, akl, YL, eG, Mq, up, JI, dH, fc, hl, awv, Qq, kt, pD, aoD, as, bQA, bJy, nr, eE, eF, ir, bQF, cm, qJ, eY, bw, lt, bQl, ii, AV, vA, mx, aiM, hh, Vj, dD, yG, n, oY, uE, brY, b, no, Hx, ec, pe, jz, zw, aAh, eV, hn, bQx, awL, xP, A, fJ, lb, bQq, QP, yq, amo, bQL, cA, bQK, at, pg, ut, eo, fL, bdR, cB, B, bQt, NO, dA, wL, eX, fF, GC, afK, v, aNG, Qo, V, eJ, aPW, bQG, yw, DK, aLS, sl, ld, bau, ae, lr, aFs, hM, vR, ev, acB, bu, aaD, t, aff, ck, zJ, Ty, wD, gX, hO, auT, ro, JH, yH, vi, kV, MH, dM, df, bQb, amQ, OR, wV, eq, Pm, hd, CJ, afm, ds, Hk, qh, LA, ab, hD, rN, JK, bQw, sj, d, je, wc, NH, aFt, rM, dN, Z, Hv, D, gk, bQE, akQ, yE, Ny, dO, wz, gZ, aef, y, CW, hv, aC, fR, atj, al, gf, Qp, aMB, bOI, AW, bOn, xE, Bc, bQo, pX, bW, M, K, aZS, DY, dF, eL, bNG, bzH, HI, axu, mQ, hN, aB, g, pL, bwe, uC, aJX, ly, xK, rq, aDs, uy, U, uo, dW, m, bi, sQ, sw, gp, lo, aw, fN, BH, BF, aBl, eh, asS, KH, Lv, cU, Ne, aFi, wb, ap, Ni, alw, Og, nN, jT, cM, Zb, amt, bMc, kT, ar, W, bo, mg, JM, hs, it, L, aAf, fM, fG, jx, aee, wo, hz, j, il, pd, eA, J, o, MR, nX, jZ, apJ, qG, bq, bPQ, kl, Y, Pg, r, en, wA, iK, bmS, brZ, sn, c, aQd, hc, Uq, wR, rK, dy, bQm, EY, fD, gx, sY, kh, id, aFe, bxK, akd, aZ, LX, em, uO, bQH, bS, ah, pl, hF, iJ, aAq, dP, fU, NY, oj, vB, ji, Dn, asU, NM, qE, asn, aVZ, eH, Sn, cr, nf, gE, hp, Bv, Lj, aFb, eB, sG, aCl, ei, hE, vt, bNu, bJx, pf, eb, zh, aR, dQ, H, eN, bQv, byv, hm, DA, aAu, LC, aW, lh, Rr, HA, aOW, eM, iT, Jp, uJ, mk, gn, R, gD, aEj, bQp, My, bQu, Mi, ct, qN, fr, aPb, ai, Vs, cc, hw, qg, ql, rl, kl, h, jy, dL, bam, hB, he, CM, aq, mo, dw, kk, op, Wt, NL, hk, uw, bm, uz, te, va, eQ, amb, bni, bL, Dw, lk, rG, bll, eR, dk, cR, C, ady, mj, ga, aCY, mP, uT, qp, afQ, aT, vH, bK, adV, an, KU, af, alk, s, aZB, oV, ND, yL, iU, akY, aDm, f, aDQ, ajl, wg, pZ, alt, vZ, aGo, AH, fP, CZ, lJ, hS, Kd, bly, Ke, aD, ha, lD, jE, mC, dZ, XB, KL, qC, av, F, ot, ib, xl, oz, s, Os, jo, aFl, vO, uQ, Di, bPq, aaB, eZ, aFr, xv, bQr, aF, bPK, ek, LB, zr, atM, bE, aS, aGg, bon, Lz, pk, be, XO, X, us); #c1(IL1F10) c2(NP_(15945) c3(interleukin-1 family member 10) c4(224851, 224852) c5(224853, 224854, 224855, 224856, 224857, 224858, 224859, 224860, 224861, 224862, 224863, 224864, 224865, 224866, 224867, 224868, 224869, 224870, 224871, 224872, 224873, 224874) c6(224875, 224876, 224877, 224878, 224879, 224880) c7(mR, ac, aw, e, iy, j, y, eu); #c1(IL1R1) c2(XP_011509420) c3(interleukin-1 receptor type 1 isoform X1) c4(299981, 299982, 299983, 299984, 299985) c5(299986, 299987, 299988, 299989, 299990, 299991, 299992, 299993, 299994, 299995) c6( ) c7(bM, T, fR, e, al, fd, cL, bW, dV, Ds, K, eN, aW, axu, mQ, vs, pD, eF, uC, aZx, vy, uo, m, eg, nD, dD, sQ, vE, ht, n, aj, oY, eh, ai, rl, h, eV, Gw, dL, ey, A, mR, XE, ap, ge, fe, cM, Wt, kT, uz, ut, bPV, bL, Dw, fM, rG, dA, fG, eX, aA, cz, fF, hy, v, j, C, V, eA, J, ga, mP, jZ, aT, af, kl, iU, bg, iy, t, pb, iQ, ac, and, fD, ao, Gl, sY, jh, ah, jE, eq, XB, hX, dP, wD, F, oW, hD, rN, vB, lQ, nJ, pq, qE, sj, uQ, DI, atJ, BN, eH, cr, eZ, iV, gk, MJ, Gu, y, ra, ne, rk, DB); #c1(IL1R1) c2(XP_011509422) c3(interleukin-1 receptor type 1 isoform X3) c4( ) c5(299996, 299997, 299998, 299999, 300000, 300001, 300002, 300003, 300004, 300005, 300006, 300007, 300008, 300009, 300010, 300011, 300012, 300013, 300014, 300015, 300016, 300017, 300018, 300019) c6( ) c7(bM, T, fR, e, al, fd, cL, bW, dV, Ds, K, eN, aW, axu, mQ, vs, pD, eF, uC, aZx, vy, uo, m, eg, nD, dD, sQ, vE, ht, n, aj, oY, eh, ai, rl, h, eV, Gw, dL, ey, A, mR, XE, ap, ge, fe, cM, Wt, kT, uz, ut, bPV, bL, Dw, fM, rG, dA, fG, eX, aA, cz, fF, hy, v, j, C, V, eA, J, ga, mP, jZ, aT, af, kl, iU, bg, iy, t, pb, iQ, ac, aQd, fD, ao, Gl, sY, jh, ah, jE, eq, XB, hX, dP, wD, F, oW, hD, rN, vB, lQ, nJ, pq, qE, sj, uQ, DI, atJ, BN, eH, cr, eZ, iV, gk, MJ, Gu, y, ra, ne, rk, DB); #c1(IL1R2) c2(NP_004624) c3(interleukin-1 receptor type 2 isoform 1 precursor) c4( ) c5(148272, 148273, 148274, 148275, 148276, 148277, 148278, 148279, 148280, 148281) c6( ) c7(bM, sY, dD, Dw, fR, sQ, iW, jx, fd, eq, apt, dP, rl, Ds, lQ, hH, af, mR, mQ, hl, eZ, kp, iV, t, uC, afc, sG, uo, uz, ut, fD, y); #c1(IL1R2) c2(XP_006712799) c3(interleukin-1 receptor type 2 isoform X3) c4(286840) c5(286841, 286842) c6( ) c7(bM, sY, dD, Dw, fR, sQ, iW, jx, fd, eq, apt, dP, rl, Ds, lQ, hH, af, mR, mQ, hl, eZ, kp, iV, t, uC, afc, sG, uo, uz, ut, fD, y); #c1(IL1R2) c2(XP_011510108) c3(interleukin-1 receptor type 2 isoform X7) c4(300703) c5( ) c6( ) c7(bM, sY, dD, Dw, fR, sQ, iW, jx, fd, eq, apt, dP, rl, Ds, lQ, hH, af, mR, mQ, hl, eZ, kp, iV, t, uC, afc, sG, uo, uz, ut, fD, y); #c1(IL1R2) c2(XP_011510109) c3(interleukin-1 receptor type 2 isoform X8) c4( ) c5(300704, 300705, 300706, 300707, 300708, 300709) c6( ) c7(bM, sY, dD, Dw, fR, sQ, iW, jx, fd, eq, apt, dP, rl, Ds, lQ, hH, af, mR, mQ, hl, eZ, kp, iV, t, uC, afc, sG, uo, uz, ut, fD, y); #c1(IL1RAP) c2(NP_001161401) c3(interleukin-1 receptor accessory protein isoform 1 precursor) c4(65700, 65701, 65702) c5(65703, 65704, 65705, 65706, 65707, 65708, 65709, 65710, 65711, 65712, 65713, 65714, 65715, 65716, 65717, 65718, 65719, 65720, 65721, 65722) c6( ) c7(dP, av, ga, jv, eJ, fR, sQ, A, aw, hl, nf, uo, ut, v, j, K, lQ, bw, bt, B, br); #c1(IL1RAP) c2(NP_001161402) c3(interleukin-1 receptor accessory protein isoform 2 precursor) c4(65723) c5(65724, 65725, 65726, 65727, 65728, 65729, 65730, 65731, 65732, 65733, 65734, 65735, 65736, 65737, 65738, 65739, 65740, 65741, 65742, 65743, 65744, 65745) c6( ) c7(dP, av, ga, jv, eJ, fR, sQ, A, aw, hl, nf, uo, ut, v, j, K, lQ, bw, bt, B, br); #c1(IL1RAP) c2(NP_001161403) c3(interleukin-1 receptor accessory protein isoform 3 precursor) c4(65746) c5(65747, 65748, 65749, 65750, 65751, 65752, 65753, 65754, 65755, 65756) c6( ) c7(dP, av, ga, jv, eJ, fR, sQ, A, aw, hl, nf, uo, ut, v, j, K, lQ, bw, bt, B, br); #c1(IL1RAPL1) c2(XP_005274498) c3(interleukin-1 receptor accessory protein-like 1 isoform X1) c4(285111) c5(285112, 285113, 285114, 285115, 285116, 285117, 285118, 285119, 285120, 285121) c6( ) c7(sY, VX, bQM, aQc, dB, Y, bCO, nf, amj, nA, rz, uC, bN); #c1(IL1RAPL2) c2(NP_059112) c3(X-linked interleukin-1 receptor accessory protein-like 2 precursor) c4(193091, 193092) c5(193093, 193094, 193095, 193096, 193097, 193098, 193099, 193100, 193101, 193102, 193103, 193104, 193105) c6( ) c7(Bv, c, lb, nf, K, rz, bN, vi); #c1(IL1RAPL2) c2(XP_011529207) c3(X-linked interleukin-1 receptor accessory protein-like 2 isoform X1) c4( ) c5(324624, 324625, 324626, 324627, 324628, 324629) c6( ) c7(Bv, c, lb, nf, K, rz, bN, vi); #c1(IL1RL1) c2(NP_001269337) c3(interleukin-1 receptor-like 1 isoform 3) c4( ) c5(101155, 101156, 101157, 101158, 101159, 101160, 101161) c6( ) c7(bM, dD, fR, ea, aYJ, e, jx, aw, fd, amj, BH, v, j, eq, C, eJ, axC, rl, aDp, aa, Nr, sp, mQ, fe, po, aDm, hp, iV, eE, Kp, f, adz, Nq, zJ, D, EW, ck, iO, iT, AH, JM, cj, wR, bQN, y, bt, rk, aZN); #c1(IL1RL1) c2(NP_003847) c3(interleukin-1 receptor-like 1 isoform 2 precursor) c4(141368, 141369, 141370) c5(141371, 141372, 141373, 141374, 141375, 141376, 141377, 141378, 141379, 141380, 141381, 141382, 141383, 141384, 141385, 141386, 141387, 141388, 141389, 141390, 141391, 141392, 141393, 141394, 141395, 141396, 141397, 141398, 141399, 141400, 141401, 141402) c6(141403) c7(bM, dD, fR, ea, aYJ, e, jx, aw, fd, amj, BH, v, j, eq, C, eJ, axC, rl, aDp, aa, Nr, sp, mQ, fe, po, aDm, hp, iV, eE, Kp, f, adz, Nq, zJ, D, EW, ck, iO, iT, AH, JM, cj, wR, bQN, y, bt, rk, aZN); #c1(IL1RL1) c2(NP_057316) c3(interleukin-1 receptor-like 1 isoform 1 precursor) c4(190074, 190075, 190076) c5(190077, 190078, 190079, 190080, 190081, 190082, 190083, 190084) c6( ) c7(bM, dD, fR, ea, aYJ, e, jx, aw, fd, amj, BH, v, j, eq, C, eJ, axC, rl, aDp, aa, Nr, sp, mQ, fe, po, aDm, hp, iV, eE, Kp, f, adz, Nq, zJ, D, EW, ck, iO, lT, AH, JM, cj, wR, bQN, y, bt, rk, aZN); #c1(IL1RL2) c2(NP_003845) c3(interleukin-1 receptor-like 2 precursor) c4( ) c5(141345, 141346, 141347, 141348, 141349, 141350, 141351, 141352) c6( ) c7(bM, rl, cr, cj, hp, an, rk); #c1(IL1RL2) c2(XP_005264092) c3(interleukin-1 receptor-like 2 isoform X5) c4(278664) c5(278665, 278666) c6( ) c7(bM, rl, cr, cj, hp, an, rk); #c1(IL1RL2) c2(XP_006712883) c3(interleukin-1 receptor-like 2 isoform X9) c4( ) c5(286902) c6( ) c7(bM, rl, cr, cj, hp, an, rk); #c1(IL1RL2) c2(XP_011510398) c3(interleukin-1 receptor-like 2 isoform X8) c4(300946) c5(300947, 300948, 300949, 300950, 300951, 300952) c6( ) c7(bM, rl, cr, cj, hp, an, rk); #c1(IL1RL2) c2(XP_011510400) c3(interleukin-1 receptor-like 2 isoform X1I) c4(300953) c5(300954, 300955, 300956, 300957, 300958, 300959) c6( ) c7(bM, rl, cr, cj, hp, an, rk); #c1(IL1RN) c2(NP_000568) c3(interleukin-1 receptor antagonist protein isoform 3) c4(12773, 12774) c5(12775, 12776, 12777, 12778, 12779, 12780, 12781) c6( ) c7(uB, T, ea, fd, aeu, ci, aY, xy, aE, rw, dV, Ds, hH, aa, li, yo, az, ih, vs, ba, fT, gt, Jr, cj, Zn, Dt, k, eg, cE, aEw, LT, bOO, aYZ, vE, iB, ht, aj, qW, zo, ey, pl, mR, sp, aRL, ge, fe, CQ, RB, ajd, aA, cz, aDD, bX, q, akU, cy, aAD, dl, eu, bA, bf, fO, bg, iy, rs, pb, OW, ac, iQ, ig, bt, bez, ao, gV, iW, jh, dG, uu, ad, wD, hX, abq, oW, lQ, DI, atJ, lx, azS, cW, ff, brp, EZ, Sm, iV, bz, amK, ME, lf, lC, Pk, ne, rk, ra, oO, bM, bQT, if, e, cL, bh, Ul, Tz, x, Yy, bc, bs, akl, YL, up, JI, dH, hl, kt, pD, as, eE, lt, Kv, ii, AV, vA, mx, hh, aFf, Vj, dD, gU, n, uE, zw, ec, xP, A, fJ, Ib, yq, bQL, cA, bQU, at, ut, eo, fL, cB, B, dA, fF, v, V, aPW, rS, sl, ld, ae, t, ck, wl, ro, dM, df, bQb, eq, qi, LA, ab, HD, rN, JK, bQw, d, Z, gk, yE, Ny, bQS, gZ, MJ, y, hv, fR, atj, al, bQR, CH, qj, Qp, xE, bW, M, K, wt, DY, eL, bzH, axu, qS, asD, aB, g, pL, bwe, uC, rq, uy, uo, m, sl, sw, lo, aw, BH, apt, aBl, pM, eh, cU, wb, ap, Ni, alw, cM, Zj, amt, bMc, kT, ar, xO, JM, Pr, it, aAf, fM, fG, jx, aee, hz, j, il, eA, J, o, nX, jZ, qG, kl, Y, r, en, bN, sn, aQd, hc, cS, dy, fD, sY, aFe, akd, aZ, LX, em, bQH, bS, ah, pl, bKZ, aAq, dP, NY, ji, adx, qE, cr, hp, TA, aFb, sG, ei, bol, G, bJx, eb, aTy, bwS, dY, eN, byv, UC, UO, aAu, aW, aOW, eM, Jp, mk, R, bQP, aEj, bQp, qN, aPb, ai, Vs, cc, rl, h, dL, hB, he, aq, op, Wt, vb, bm, uz, aDE, bni, bL, Dw, rG, eR, C, ga, mP, bQQ, qp, aT, bK, an, af, s, iU, yL, akY, aDm, ajl, alt, AH, CZ, aD, ha, jE, mC, XB, kL, qC, av, F, oz, Os, jo, pq, uQ, eZ, aFr, aF, bPK, ek, LB, zr, axs, bE, atM, z, X); #c1(IL1RN) c2(NP_776213) c3(interleukin-1 receptor antagonist protein isoform 2) c4(251002) c5(251003, 251004, 251005, 251006, 251007, 251008) c6( ) c7(uB, T, ea, fd, aeu, ci, aY, xy, aE, rw, dV, Ds, hH, aa, li, yo, az, ih, vs, ba, fT, gt, Jr, cj, Zn, Dt, k, eg, cE, aEw, LT, bQO, aYZ, vE, iB, ht, aj, qW, zo, ey, pl, mR, sp, aRL, ge, fe, CQ, RB, ajd, aA, cz, aDD, bX, q, akU, cy, aAD, dl, eu, bA, bf, fO, bg, iy, rs, pb, OW, ac, iQ, ig, bt, blz, ao, gV, iW, jh, dG, uu, ad, wD, hX, abq, oW, lQ, DI, atJ, lx, azS, cW, ff, brp, EZ, Sm, iV, bz, amK, ME, lf, lC, Pk, ne, rk, ra, oO, bM, bQT, if, e, cL, bh, UI, Tz, x, Yy, bc, bs, akl, YL, up, JI, dH, hl, kt, pD, as, BE, lt, Kv, ii, AV, vA, mx, hh, aFf, Vj, dD, gU, n, uE, zw, ec, xP, A, fJ, lb, yq, bQL, cA, bQU, at, ut, eo, fL, cB, B, dA, fF, v, V, aPW, rS, sl, ld, ae, t, ck, wl, ro, dM, df, bQb, eq, qi, LA, ab, hD, rN, JK, bQw, d, Z, gk, yE, Ny, bQS, gZ, MJ, y, hv, fR, atj, al, bQR, CH, qj, Qp, xE, bW, M, K, wt, DY, eL, bzH, axu, qS, asD, aB, g, pL, bwe, uC, rq, uy, uo, m, sQ, sw, lo, aw, BH, apt, aBl, pM, eh, cU, wb, ap, Ni, alw, cM, Zj, amt, bMc, kT, ar, xO, JM, Pr, it, aAf, fM, fG, jx, aee, hz, j, il, eA, J, o, nX, jZ, qG, kl, Y, r, en, bN, sn, and, hc, cS, dy, fD, sY, aFe, akd, aZ, LX, em, bQH, bS, ah, pl, bKZ, aAq, dP, NY, ji, adx, qE, cr, hp, TA, aFb, sG, ei, bol, G, bJx, eb, aTy, bwS, dY, eN, byv, UC, UO, aAu, aW, aOW, eM, Jp, mk, R, bQP, aEj, bQp, qN, aPb, ai, Vs, cc, rl, h, dL, hB, he, aq, op, Wt, vb, bm, uz, aDE, bni, bL, Dw, rG, eR, C, ga, mP, bQQ, qp, aT, bK, an, af, s, iU, yL, akY, aDm, ajl, aJt, AH, CZ, aD, ha, jE, mC, XB, kL, qC, av, F, oz, Os, jo, pq, uQ, eZ, aFr, aF, bPK, ek, LB, zr, axs, bE, atM, z, X); #c1(IL1RN) c2(NP_776214) c3(interleukin-1 receptor antagonist protein isoform 1 precursor) c4(251009) c5(251010, 251011, 251012, 251013, 251014, 251015) c6( ) c7(uB, T, ea, fd, aeu, ci, aY, xy, aE, rw, dV, Ds, hH, aa, li, yo, az, ih, vs, ba, fT, gt, Jr, cj, Zn, Dt, k, eg, cE, aEw, LT, bQO, aYZ, vE, iB, ht, aj, qW, zo, ey, pl, mR, sp, aRL, ge, fe, CQ, RB, ajd, aA, cz, aDD, bX, q, akU, cy, aAD, dl, eu, bA, bf, fO, bg, iy, rs, pb, OW, ac, iQ, ig, bt, blz, ao, gV, iW, jh, dG, uu, ad, wD, hX, abq, oW, lQ, DI, atJ, lx, azS, cW, ff, brp, EZ, Sm, iV, bz, amK, ME, lf, lC, Pk, ne, rk, ra, oO, bM, bQT, if, e, cL, bh, UI, Tz, x, Yy, bc, bs, akl, YL, up, JI, dH, hl, kt, pD, as, BE, lt, Kv, ii, AV, vA, mx, hh, aFf, Vj, dD, gU, n, uE, zw, ec, xP, A, fJ, lb, yq, bQL, cA, bQU, at, ut, eo, fL, cB, B, dA, fF, v, V, aPW, rS, sl, ld, ae, t, ck, wO, ro, dM, df, bQb, eq, qi, LA, ab, hD, rN, JK, bQw, d, Z, gk, yE, Ny, bQS, gZ, MJ, y, hv, fR, atj, al, bQR, CH, qj, Qp, xE, bW, M, K, wt, DY, eL, bzH, axu, qS, asD, aB, g, pL, bwe, uC, rq, uy, uo, m, sQ, sw, lo, aw, BH, apt, aBl, pM, eh, cU, wb, ap, Ni, alw, cM, Zj, amt, bMc, kT, ar, xO, JM, Pr, it, aAf, fM, fG, jx, aee, hz, j, il, eA, J, o, nX, jZ, qG, kl, Y, r, en, bN, sn, and, hc, cS, dy, fD, sY, aFe, akd, aZ, LX, em, bQH, bS, ah, pl, bKZ, aAq, dP, NY, ji, adx, qE, cr, hp, TA, aFb, sG, ei, bol, G, bJx, eb, aTy, bwS, dY, eN, byv, UC, UO, aAu, aW, aOW, eM, Jp, mk, R, bQP, aEj, bQp, qN, aPb, ai, Vs, cc, rl, h, dL, hB, he, aq, op, Wt, vb, bm, uz, aDE, bni, bL, Dw, rG, eR, C, ga, mP, bQQ, qp, aT, bK, an, af, s, iU, yL, akY, aDm, ajl, aJt, AH, CZ, aD, ha, jE, mC, XB, kL, qC, av, F, oz, Os, jo, pq, uQ, eZ, afr, aF, bPK, ek, LB, zr, axs, bE, atM, z, X); #c1(IL1RN) c2(XP_005263718) c3(interleukin-1 receptor antagonist protein isoform X1) c4( ) c5(278512, 278513, 278514, 278515, 278516) c6( ) c7(uB, T, ea, fd, aeu, ci, aY, xy, aE, rw, dV, Ds, hH, aa, li, yo, az, ih, vs, ba, fT, gt, Jr, cj, Zn, Dt, k, eg, cE, aEw, LT, bOO, aYZ, vE, iB, ht, aj, qW, zo, ey, pl, mR, sp, aRL, ge, fe, CQ, RB, ajd, aA, cz, aDD, bX, q, akU, cy, aAD, dl, eu, bA, bf, fO, bg, iy, rs, pb, OW, ac, iQ, ig, bt, bez, ao, gV, iW, jh, dG, uu, ad, wD, hX, abq, oW, lQ, DI, atJ, lx, azS, cW, ff, brp, EZ, Sm, iV, bz, amK, ME, lf, lC, Pk, ne, rk, ra, oO, bM, bQT, if, e, cL, bh, UI, Tz, x, Yy, bc, bQs, akl, YL, up, JI, dH, hl, kt, pD, as, eE, lt, Kv, ii, AV, vA, mx, hh, aFf, Vj, dD, gU, n, uE, zw, ec, xP, A, fJ, lb, yq, bQL, cA, bQU, at, ut, eo, fL, cB, B, dA, fF, v, V, aPW, rS, sl, ld, ae, t, ck, wl, ro, dM, df, bQb, eq, qi, LA, ab, hD, rN, JK, bQw, d, Z, gk, yE, Ny, bQS, gZ, MJ, y, hv, FR, atj, al, bQR, CH, qj, Qp, xE, bW, M, K, wt, DY, eL, bzH, axu, qS, asD, aB, g, pL, bwe, uC, rq, uy, uo, m, sQ, sw, lo, aw, BH, apt, aBl, pM, eh, cU, wb, ap, Ni, alw, cM, Zj, amt, bMc, kT, ar, xO, JM, Pr, it, aAf, fM, fG, jx, aee, hz, j, il, eA, J, o, nX, jZ, qG, kl, Y, r, en, bN, sn, and, hc, cS, dy, fD, sY, aFe, akd, aZ, LX, em, bQH, bS, ah, pl, bKZ, aAq, dP, NY, ji, adx, qE, cr, hp, TA, aFb, sG, ei, bol, G, bJx, eb, aTy, bwS, dY, eN, byv, UC, UO, aAu, aW, aOW, eM, Jp, mk, R, bQP, aEj, blp, qN, aPb, ai, Vs, cc, rl, h, dL, hB, he, aq, op, Wt, vb, bm, uz, aDE, bni, bL, Dw, rG, eR, C, ga, mP, bQQ, qp, aT, bK, an, af, s, iU, yL, akY, aDm, ajl, aJt, AH, CZ, aD, ha, jE, mC, XB, KL, qC, av, F, oz, Os, jo, pq, uQ, eZ, aFr, aF, bPK, eK, LB, zr, axs, bE, atM, z, X); #c1(IL2) c2(NP_000577) c3(interleukin-2 precursor) c4(12951) c5(12952, 12953, 12954, 12955, 12956, 12957, 12958, 12959, 12960, 12961, 12962, 12963, 12964, 12965, 12966, 12967, 12968, 12969, 12970, 12971, 12972, 12973, 12974, 12975, 12976, 12977, 12978, 12979, 12980, 12981, 12982, 12983, 12984, 12985, 12986, 12987, 12988, 12989, 12990, 12991, 12992, 12993, 12994, 12995, 12996, 12997, 12998, 12999, 13000, 13001, 13002, 13003, 13004, 13005, 13006, 13007, 13008, 13009, 13010, 13011, 13012, 13013, 13014, 13015, 13016, 13017, 13018, 13019, 13020, 13021, 13022, 13023, 13024, 13025, 13026, 13027, 13028, 13029, 13030, 13031, 13032, 13033, 13034, 13035, 13036, 13037, 13038, 13039, 13040, 13041, 13042, 13043, 13044, 13045, 13046, 13047, 13048, 13049, 13050, 13051, 13052, 13053, 13054, 13055, 13056, 13057, 13058, 13059, 13060, 13061, 13062, 13063, 13064, 13065, 13066, 13067, 13068, 13069, 13070, 13071, 13072, 13073, 13074, 13075, 13076, 13077, 13078, 13079, 13080, 13081, 13082, 13083, 13084, 13085, 13086, 13087, 13088, 13089, 13090, 13091, 13092, 13093, 13094, 13095, 13096, 13097, 13098, 13099, 13100, 13101, 13102, 13103, 13104, 13105, 13106, 13107, 13108, 13109, 13110, 13111, 13112, 13113, 13114, 13115, 13116, 13117, 13118, 13119, 13120, 13121, 13122, 13123, 13124, 13125, 13126, 13127, 13128, 13129, 13130, 13131) c6( ) c7(T, ea, fd, ci, aY, amW, aE, rw, dV, Ds, Bi, hu, hH, azh, cp, VF, aa, yo, hC, az, ba, fT, iA, nB, avZ, dc, cj, k, act, eg, aYZ, ht, aj, qW, ey, bEg, mR, ge, fe, CQ, zZ, adz, aBs, bbz, apO, dB, Ei, aA, bX, wy, q, zn, cy, sX, dl, fO, bg, lo, pb, Cv, ov, cT, pa, OW, iM, iQ, ig, RW, bt, ao, On, aCA, iW, jh, dG, fu, ad, wD, hX, oW, iY, lQ, bQX, gj, qZ, asf, ff, avG, iV, bz, aYt, alx, akC, ra, iu, ne, bM, if, e, bB, cL, bh, asr, Tz, x, vV, bc, eG, hl, kt, eE, cm, bw, eY, vA, oy, mx, aiu, dD, yG, n, pe, eV, bQd, bav, A, fJ, po, at, ut, eo, fL, B, dA, eX, v, V, ld, ae, aFs, hM, iZ, axW, ev, aFK, t, ck, kP, aGi, bdP, HK, yH, bQW, vi, eq, qi, Fc, hd, py, afm, bQV, aCJ, ds, qh, hD, rN, d, je, hG, Z, gk, gy, y, hv, pC, ng, aC, fR, azf, atj, al, gf, qj, lR, wa, AW, bW, pX, rB, K, DY, eL, bNG, mH, aCM, axu, asD, md, LZ, aB, g, uC, iP, aJw, uy, uo, dW, m, NK, sQ, sw, lo, fN, BH, ft, kH, cU, aDp, dx, gi, aJG, aiS, gg, ph, WL, Pr, fM, fG, jx, aSE, j, pd, eA, J, o, er, XX, bq, hK, xR, Y, r, en, aER, iK, bN, hc, dy, EY, fD, LS, gx, sY, bmQ, aFe, em, auq, bS, ah, hF, vB, aNp, ji, lR, vj, cr, hp, BG, jg, bNW, Nq, Bv, asq, aFb, hE, eb, aR, il, pk, azx, DA, aAu, aW, iT, eM, Jp, aEj, bwX, fr, ct, qN, aPb, ai, eW, BI, h, dL, Nr, asp, CM, aq, kp, mo, gG, kk, op, Wt, NL, uz, bCS, bL, Dw, eR, OY, C, ze, ga, blC, mP, nv, Vr, qp, aT, UN, an, af, s, iU, oV, hf, sS, aXm, aJt, im, bH, hS, Kd, aD, jE, pz, mC, kL, LH, qC, av, F, oz, uQ, eZ, bxd, aF, gu, aXs, es, eU, zr, axs, bE, z, dJ, uR, aS, fh, X); #c1(IL20) c2(NP_061194) c3(interleukin-20 precursor) c4(199901, 199902) c5(199903, 199904, 199905, 199906, 199907, 199908, 199909, 199910, 199911, 199912, 199913, 199914, 199915, 199916) c6( ) c7(bM, Dw, dD, eR, fR, ea, lH, jx, qj, jE, eq, ai, hX, xE, aE, ws, M, aT, cU, cy, Ds, h, K, lQ, bA, A, aW, fO, cr, eZ, iV, eE, bz, DF, wg, ac, eM, bbx, E, Dt, byK, y, pC); #c1(IL20) c2(XP_005273209) c3(interleukin-20 isoform X1) c4( ) c5(284335, 284336) c6( ) c7(bM, Dw, dD, eR, fR, ea, lH, jx, qj, jE, eq, ai, hX, xE, aE, ws, M, aT, cU, cy, Ds, h, K, lQ, bA, A, aW, fO, cr, eZ, iV, BE, bz, DF, wg, ac, eM, bbx, E, Dt, byK, y, pC); #c1(IL20RA) c2(NP_001265651) c3(interleukin-20 receptor subunit alpha isoform 2) c4(99123) c5( ) c6( ) c7(aW, V, Y, cr, aT); #c1(IL20RA) c2(NP_055247) c3(interleukin-20 receptor subunit alpha isoform 1 precursor) c4(180039, 180040) c5(180041, 180042, 180043, 180044, 180045, 180046, 180047, 180048, 180049, 180050, 180051, 180052, 180053, 180054, 180055, 180056, 180057, 180058) c6( ) c7(aW, V, Y, cr, aT); #c1(IL20RA) c2(XP_011534206) c3(interleukin-20 receptor subunit alpha isoform X2) c4(330527) c5(330528, 330529, 330530, 330531, 330532, 330533) c6( ) c7(aW, V, Y, cr, aT); #c1(IL20RB) c2(NP_653318) c3(interleukin-20 receptor subunit beta precursor) c4(239576) c5(239577, 239578, 239579, 239580, 239581, 239582, 239583) c6( ) c7(Y, j, cr, eM); #c1(IL20RB) c2(XP_006713728) c3(interleukin-20 receptor subunit beta isoform X2) c4( ) c5(287309, 287310, 287311, 287312, 287313) c6( ) c7(Y, j, cr, eM); #c1(IL20RB) c2(XP_011511212) c3(interleukin-20 receptor subunit beta isoform X1) c4(301695) c5( ) c6( ) c7(Y, j, cr, eM); #c1(IL21) c2(NP_068575) c3(interleukin-21 isoform 1 precursor) c4(209459) c5(209460, 209461, 209462, 209463, 209464, 209465, 209466, 209467, 209468, 209469, 209470, 209471, 209472, 209473, 209474, 209475, 209476, 209477, 209478, 209479, 209480, 209481, 209482, 209483, 209484, 209485, 209486, 209487, 209488, 209489, 209490, 209491, 209492, 209493, 209494, 209495, 209496, 209497, 209498, 209499, 209500, 209501, 209502, 209503, 209504, 209505, 209506, 209507, 209508, 209509, 209510, 209511, 209512, 209513, 209514, 209515, 209516, 209517, 209518, 209519, 209520, 209521, 209522, 209523, 209524, 209525, 209526, 209527, 209528, 209529, 209530, 209531, 209532, 209533, 209534, 209535, 209536, 209537, 209538, 209539, 209540, 209541, 209542, 209543, 209544, 209545, 209546, 209547, 209548, 209549, 209550, 209551, 209552) c6(209553) c7(bM, T, fR, fd, bB, cL, xE, Ds, DY, aW, HA, hl, hN, eE, cm, cj, m, eg, fN, aj, cU, eV, aNj, A, fe, aq, kk, Wt, at, ajl, eo, fL, Pr, bL, Ob, Dw, fM, dA, lH, eX, aA, abC, V, eA, o, J, aT, hk, Y, bg, lC, lo, t, pb, lY, OW, ac, iM, iQ, of, hl, aNk, jh, jE, mC, hX, ahr, ji, d, je, yx, eZ, cr, dN, aF, hp, jg, iV, iR, aGj, ek, eU, bE, MJ, gy, y, hv); #c1(IL21R) c2(NP_068570) c3(interleukin-21 receptor isoform 1 precursor) c4(209440) c5( ) c6( ) c7(bM, hl, bQZ, dA, oe, jh, fd, V, abC, mC, bGD, A, je, eZ, dN, fe, eE, bQY, Cq, iM, ac, eU, cj, eo, fL, y, zA); #c1(IL21R) c2(XP_011544159) c3(interleukin-21 receptor isoform X1) c4( ) c5(343320, 343321, 343322, 343323, 343324, 343325, 343326, 343327, 343328, 343329, 343330, 343331, 343332, 343333) c6( ) c7(bM, hl, bQZ, dA, oe, jh, fd, V, abC, mC, bGD, A, je, eZ, dN, fe, eE, bQY, Cq, iM, ac, eU, cj, eo, fL, y, zA); #c1(IL22) c2(NP_065386) c3(interleukin-22 precursor) c4(204640) c5(204641, 204642, 204643, 204644, 204645, 204646, 204647, 204648, 204649, 204650) c6( ) c7(oO, bM, T, FR, fd, bB, zh, ci, qj, bh, xE, ws, dV, Ds, Bi, hH, aW, hl, eE, cm, ba, lt, eM, Jr, cj, m, qR, fN, aj, qW, ai, cU, h, aNj, A, mR, fe, aq, kk, jT, Wt, ar, Pa, bbx, at, eo, fL, B, bL, Dw, fM, dA, oe, jx, aA, v, ON, C, V, pd, q, J, eA, DK, aT, qU, cy, Cy, Y, bg, r, aMA, lo, aDm, en, t, ua, ac, AH, dT, wR, CZ, yH, fD, byK, hl, Ke, aNk, jh, wV, jE, atK, mC, hX, oz, ds, hD, ahr, ji, lQ, d, Zf, yx, ff, cr, eZ, dN, aF, hp, jg, iV, bz, eK, eU, bE, z, MJ, agl, hE, X, y, pC); #c1(IL22RA2) c2(NP_443194) c3(interleukin-22 receptor subunit alpha-2 isoform 1 precursor) c4(229788) c5(229789, 229790, 229791, 229792, 229793, 229794) c6( ) c7(hH, fq); #c1(IL22RA2) c2(NP_851826) c3(interleukin-22 receptor subunit alpha-2 isoform 2 precursor) c4(254645) c5(254646, 254647, 254648, 254649, 254650, 254651) c6( ) c7(hH, fq); #c1(IL22RA2) c2(NP_851827) c3(interleukin-22 receptor subunit alpha-2 isoform 3 precursor) c4(254652) c5(254653, 254654, 254655, 254656, 254657, 254658) c6( ) c7(hH, fq); #c1(IL23A) c2(XP_011536779) c3(interleukin-23 subunit alpha isoform X1) c4(334449) c5(334450, 334451, 334452, 334453, 334454, 334455) c6( ) c7(oO, bM, T, fR, if, al, fd, ci, mF, wa, xE, aE, dV, K, hH, LG, eL, bc, aoo, az, aoD, eE, bw, eM, Jr, U, m, jM, aDi, qR, fN, aj, uE, qN, qW, ai, kH, aNj, Lg, A, mR, fe, bP, eD, at, eo, fL, B, lk, dA, lH, jx, aA, v, OY, C, V, q, J, eA, mP, jZ, ld, dn, Oi, xR, Y, r, lo, t, pb, iM, c, wR, yH, fD, kZ, aNk, Hn, jE, fu, hF, mC, Pm, wD, hX, ds, oW, io, dm, hD, ji, lQ, pq, je, cr, eZ, dN, aF, iV, aGj, bz, aJx, eU, aCl, bPJ, lf, y); #c1(IL23R) c2(XP_011539091) c3(interleukin-23 receptor isoform X2) c4(337154) c5(337155, 337156, 337157, 337158, 337159) c6( ) c7(bM, T, cL, lR, rm, AW, rw, ja, Ds, hu, hH, zQ, bn, aW, bRa, HA, LZ, NR, pL, aFU, eM, Jr, mx, dW, aw, ht, bmW, fN, NT, qW, ai, ft, hi, A, mR, AQ, ge, fe, xe, cM, lx, Qu, it, Pr, B, bL, Dw, lk, aA, cz, hz, j, V, q, o, mP, aT, af, qw, Lu, aZs, Pg, r, t, pb, wR, bt, xb, zA, hF, gb, wD, hX, NS, hD, ji, lQ, yF, bRb, eZ, cr, aF, hp, aDd, iV, axs, Lj, gy, lC, ra, iu, y); #c1(IL23R) c2(XP_011539093) c3(interleukin-23 receptor isoform X1) c4(337160, 337161, 337162, 337163, 337164, 337165, 337166) c5(337167, 337168, 337169, 337170, 337171, 337172, 337173, 337174, 337175, 337176, 337177, 337178, 337179, 337180, 337181, 337182, 337183, 337184, 337185, 337186, 337187, 337188, 337189, 337190, 337191, 337192, 337193, 337194, 337195, 337196) c6( ) c7(bM, T, cL, lR, rm, AW, rw, ja, Ds, hu, hH, zQ, bn, aW, bRa, HA, LZ, NR, pL, aFU, eM, Jr, mx, dW, aw, ht, bmW, fN, NT, qW, ai, ft, hi, A, mR, AQ, ge, fe, xe, cM, lx, Qu, it, Pr, B, bL, Dw, Ik, aA, cz, hz, j, V, q, o, mP, aT, af, qw, Lu, aZs, Pg, r, t, pb, wR, bt, xb, zA, hF, gb, wD, hX, NS, HD, ji, lQ, yF, bRb, eZ, cr, aF, hp, aDd, iV, axs, Lj, gy, lC, ra, iu, y); #c1(IL24) c2(NP_001172085) c3(interleukin-24 isoform 3 precursor) c4(72374) c5(72375, 72376, 72377, 72378, 72379, 72380, 72381, 72382) c6(72383, 72384) c7(bM, T, Dw, aC, lk, if, jx, jh, aA, bB, n, qj, ci, qN, fr, q, ad, aMB, eA, x, av, ib, aE, aT, h, Ds, K, hS, ae, cp, d, iU, nO, JI, aW, Y, iZ, bg, ff, cr, zD, r, dN, aq, kp, aF, eZ, en, hf, g, t, cm, bz, QA, iR, iV, fe, ck, ov, bP, eM, iA, dB, bE, iT, cj, np, m, k, byK, y, bt, B, pC); #c1(IL24) c2(NP_001172086) c3(interleukin-24 isoform 4 precursor) c4(72385) c5(72386, 72387, 72388, 72389, 72390, 72391, 72392, 72393) c6(72394, 72395) c7(bM, T, Dw, aC, lk, if, jx, jh, aA, bB, n, qj, ci, qN, fr, q, ad, aMB, eA, x, av, ib, aE, aT, h, Ds, K, hS, ae, cp, d, iU, nO, JI, aW, Y, iZ, bg, ff, cr, zD, r, dN, aq, kp, aF, eZ, en, hf, g, t, cm, bz, QA, iR, iV, fe, ck, ov, bP, eM, iA, dB, bE, lT, cj, np, m, k, byK, y, bt, B, pC); #c1(IL24) c2(NP_006841) c3(interleukin-24 isoform 1 precursor) c4(168573) c5(168574, 168575, 168576, 168577, 168578, 168579, 168580, 168581) c6(168582, 168583) c7(bM, T, Dw, aC, lk, if, jx, jh, aA, bB, n, qj, ci, qN, fr, q, ad, aMB, eA, x, av, ib, aE, aT, h, Ds, K, hS, ae, cp, d, iU, nO, JI, aW, Y, iZ, bg, ff, cr, zD, r, dN, aq, kp, af, eZ, en, hf, g, t, cm, bz, QA, iR, iV, fe, ck, ov, bP, eM, iA, dB, bE, iT, cj, np, m, k, byK, y, bt, B, pC); #c1(IL25) c2(NP_073626) c3(interleukin-25 isoform 1 precursor) c4(213185) c5(213186, 213187, 213188, 213189, 213190, 213191) c6( ) c7(Nq, bM, Nr, aDu, eR, A, aCT, aW, z, aT, fe, po, aoj, fD, eE, aY, DF); #c1(IL25) c2(NP_758525) c3(interleukin-25 isoform 2 precursor) c4(249246) c5(249247, 249248, 249249, 249250, 249251, 249252) c6( ) c7(Nq, bM, Nr, aDu, eR, A, aCT, aW, z, aT, fe, po, aoj, fD, eE, aY, DF); #c1(IL26) c2(XP_011536870) c3(interleukin-26 isoform X1) c4(334565) c5(334566, 334567, 334568, 334569, 334570, 334571) c6( ) c7(bM, Dw, A, dV, aA, ge, fe, Ds, aF, K, iV, y, V, fq); #c1(IL27) c2(NP_663634) c3(interleukin-27 subunit alpha precursor) c4(241396) c5(241397, 241398, 241399, 241400, 241401, 241402) c6( ) c7(oO, bM, cZ, T, fM, JZ, dD, Dw, ea, ht, al, jh, fF, aj, v, eq, jE, C, V, qW, ai, J, ad, aDF, hX, eA, ds, Os, aT, Ds, bQd, K, ji, dd, hu, DY, ae, DA, A, je, aW, Y, oV, fO, cr, ge, fe, lo, iV, pL, axp, bz, ck, iM, W, anT, ig, dB, bE, eM, z, Be, at, dW, Dt, m, fD, eo, y, bt, Pr, B); #c1(IL27RA) c2(NP_004834) c3(interleukin-27 receptor subunit alpha precursor) c4(150158) c5(150159, 150160, 150161, 150162, 150163, 150164, 150165) c6( ) c7(ai, bM, dD, Dw, z, ea, Y, Ds, dW, eq, ci, fD, y, bt, af); #c1(IL2RA) c2(NP_000408) c3(interleukin-2 receptor subunit alpha precursor) c4(8633, 8634) c5(8635, 8636, 8637, 8638, 8639, 8640, 8641, 8642, 8643, 8644, 8645, 8646) c6( ) c7(bM, T, FR, bO, CH, fd, cL, Vd, ci, qj, bh, aY, amW, aR, AW, xy, xE, aE, rw, dV, aYy, Ds, azm, bRd, rB, DY, bhm, bc, aa, li, JI, aW, az, kt, hN, pL, eE, uC, cm, mK, bw, yd, ba, y, eM, Jr, iA, uy, iT, mx, cj, uo, k, m, gn, abh, aiu, sQ, aO, yG, fN, aj, fr, cd, ai, De, xC, baA, ft, cc, ql, kH, h, hn, du, ox, Nr, A, zb, fJ, fe, po, bxh, CQ, jT, xe, adz, Wt, yR, ar, RB, W, lf, JM, bm, uz, aEG, ut, eo, fL, auR, Pr, B, it, bL, Dw, fM, dA, eR, aYB, fG, wL, zN, hy, hz, OY, V, pd, eA, J, o, zn, eJ, lg, qp, aT, qG, bK, bq, hK, aFm, Cy, ae, hM, PK, bf, nO, iU, Y, fO, bg, lo, en, t, f, ik, pb, ck, cT, sS, OW, Dy, Ta, ig, yW, iM, hc, AH, fP, wR, Da, bxj, yH, byK, gT, bt, Kd, sY, bjx, iW, bRc, oA, jh, wV, pl, jE, EA, hF, mC, ad, XB, LH, hX, wD, F, oz, qh, hD, ji, bxf, lQ, fb, iL, yF, uQ, d, je, vj, ff, cr, eZ, dN, bxd, Mz, hp, iV, bz, Nq, es, Bv, atM, Lj, z, XD, aEN, hE, ra, hv, Tc); #c1(IL2RB) c2(NP_000869) c3(interleukin-2 receptor subunit beta precursor) c4(16666, 16667, 16668, 16669) c5(16670, 16671, 16672, 16673, 16674, 16675, 16676, 16677, 16678, 16679, 16680, 16681, 16682, 16683, 16684, 16685, 16686, 16687, 16688, 16689, 16690, 16691, 16692, 16693) c6( ) c7(bM, Dw, iH, dA, fR, sQ, jx, bO, aA, fd, aj, pl, hF, V, Fc, q, aR, amW, zn, dP, eA, ady, ec, aCJ, aql, kH, h, Ds, dz, bK, K, lQ, an, zy, H, hS, rB, WZ, jf, hM, eG, A, je, LC, sp, Y, xs, oV, mQ, r, vj, Ls, eZ, aF, hp, CQ, fe, pL, eE, cm, pb, adz, DF, ck, RB, iM, c, iT, Fi, uo, ut, fD, y, bt, it, B, L); #c1(IL2RG) c2(NP_000197) c3(cytokine receptor common subunit gamma precursor) c4(4422, 4423, 4424, 4425) c5(4426, 4427, 4428, 4429, 4430, 4431, 4432, 4433, 4434, 4435, 4436, 4437, 4438, 4439, 4440, 4441, 4442, 4443, 4444, 4445, 4446, 4447, 4448, 4449) c6( ) c7(bCI, T, bM, Dw, dA, bRe, bLS, jh, bB, ci, kH, h, Ds, zA, B, pq, DA, mR, je, iZ, oV, Y, CM, CF, bRf, jg, t, oU, ck, iM, dB, bE, cj, m, y, Ze); #c1(IL3) c2(NP_000579) c3(interleukin-3 precursor) c4(13141, 13142) c5(13143, 13144, 13145, 13146, 13147, 13148) c6( ) c7(bM, G, os, T, JN, fV, fd, x, Co, dV, Ds, eL, eF, cm, uC, bw, iT, oy, U, cj, k, eg, aiu, oY, aj, ai, br, eW, ke, pe, kH, h, hn, dL, DU, du, Nr, A, sp, fe, aq, po, pm, bP, kz, JM, at, Pr, B, Dw, jv, dA, eR, lH, cz, afK, bX, eA, J, zn, aT, rO, dz, j, af, bA, iU, oV, Y, ck, cT, BU, iM, bt, sY, oA, bs, jh, oZ, fu, EA, hF, ase, ad, CJ, fX, vJ, ds, oW, aZW, HD, iY, je, hG, vj, eH, cr, hp, uF, avG, iV, Nq, EW, amS, pc, zr, z, aKC, y); #c1(IL31) c2(XP_011536628) c3(interleukin-31 isoform X1) c4(334104) c5(334105, 334106, 334107, 334108, 334109, 334110, 334111, 334112) c6(334113, 334114) c7(bP, buT, iE, mQ, asL, cr, gw, dN, fe, lQ, eE); #c1(IL31RA) c2(NP_001229565) c3(interleukin-31 receptor subunit alpha isoform 2 precursor) c4( ) c5(84637) c6( ) c7(es, d, iA, bRg, aW, aT, aA, cm, v, aLA, K, m, aay, eE, t, aF, bz, fr, pl); #c1(IL31RA) c2(NP_001229566) c3(interleukin-31 receptor subunit alpha isoform 3) c4(84638) c5(84639, 84640, 84641, 84642, 84643, 84644) c6( ) c7(es, d, iA, bRg, aW, al, aA, cm, v, aLA, K, m, aay, eE, t, aF, bz, fr, pl); #c1(IL31RA) c2(NP_001229567) c3(interleukin-31 receptor subunit alpha isoform 4 precursor) c4(84645) c5(84646, 84647, 84648, 84649, 84650, 84651, 84652, 84653, 84654, 84655) c6( ) c7(es, d, iA, bRg, aW, aT, aA, cm, v, aLA, K, m, aay, eE, t, aF, bz, fr, pl); #c1(IL31RA) c2(NP_001284499) c3(interleukin-31 receptor subunit alpha isoform 6) c4(111923, 111924) c5(111925, 111926, 111927, 111928, 111929, 111930, 111931) c6( ) c7(es, d, iA, bRg, aW, aT, aA, cm, v, aLA, K, m, aay, eE, t, af, bz, fr, pl); #c1(IL31RA) c2(NP_001284501) c3(interleukin-31 receptor subunit alpha isoform 7) c4( ) c5(111932, 111933, 111934, 111935, 111936) c6( ) c7(es, d, iA, bRg, aW, aT, aA, cm, v, aLA, K, m, aay, eE, t, af, bz, fr, pl); #c1(IL31RA) c2(NP_620586) c3(interleukin-31 receptor subunit alpha isoform 1) c4(237811) c5(237812, 237813, 237814, 237815, 237816, 237817) c6( ) c7(es, d, iA, bRg, aW, aT, aA, cm, v, aLA, K, m, aay, eE, t, af, bz, fr, pl); #c1(IL31RA) c2(XP_011541444) c3(interleukin-31 receptor subunit alpha isoform X3) c4(340031) c5( ) c6( ) c7(es, d, iA, bRg, aW, aT, aA, cm, v, aLA, K, m, aay, eE, t, af, bz, fr, pl); #c1(IL31RA) c2(XP_011541446) c3(interleukin-31 receptor subunit alpha isoform X2) c4( ) c5(340032, 340033, 340034, 340035, 340036) c6( ) c7(es, d, iA, bRg, aW, aT, aA, cm, v, aLA, K, m, aay, eE, t, aF, bz, fr, pl); #c1(IL32) c2(NP_001012651) c3(interleukin-32 isoform A) c4(25120) c5(25121, 25122, 25123, 25124, 25125, 25126, 25127, 25128, 25129, 25130, 25131, 25132, 25133, 25134) c6( ) c7(T, dD, ea, aD, ht, aA, bB, fd, eq, ai, ad, hX, iu, AW, hd, aE, ds, KH, aT, io, h, hu, hB, ae, aa, aEV, d, B, mR, iU, xR, aW, Y, fJ, az, bkf, ff, fe, lo, aF, Fq, Sm, pL, jT, en, bz, g, ik, bw, Qy, eU, bE, yH, jM, y, Pr, X, nQ); #c1(IL32) c2(NP_001012652) c3(interleukin-32 isoform C) c4(25135) c5(25136, 25137, 25138, 25139, 25140, 25141, 25142, 25143, 25144, 25145, 25146, 25147, 25148, 25149, 25150, 25151) c6(25152, 25153) c7(T, dD, ea, aD, ht, aA, bB, fd, eq, ai, ad, hX, iu, AW, hd, aE, ds, KH, aT, io, h, hu, hB, ae, aa, aEV, d, B, mR, iU, xR, aW, Y, fJ, az, bkf, ff, fe, lo, aF, Fq, Sm, pL, jT, en, bz, g, iK, bw, Qy, eU, bE, yH, jM, y, Pr, X, nQ); #c1(IL32) c2(NP_001012654) c3(interleukin-32 isoform D) c4(25154) c5(25155, 25156, 25157, 25158, 25159, 25160, 25161, 25162, 25163, 25164) c6( ) c7(T, dD, ea, aD, ht, aA, bB, fd, eq, ai, ad, hX, iu, AW, hd, aE, ds, kH, aT, io, h, hu, hB, ae, aa, aEV, d, B, mR, iU, xR, aW, Y, fJ, az, bkf, ff, fe, lo, aF, Fq, Sm, pL, jT, en, bz, g, iK, bw, Qy, eU, bE, yH, jM, y, Pr, X, nQ); #c1(IL32) c2(NP_001012736) c3(interleukin-32 isoform B) c4(25193, 25194) c5(25195, 25196, 25197, 25198, 25199, 25200, 25201, 25202, 25203, 25204, 25205, 25206, 25207, 25208, 25209, 25210, 25211, 25212, 25213, 25214, 25215, 25216, 25217, 25218, 25219, 25220, 25221, 25222, 25223, 25224, 25225, 25226, 25227, 25228, 25229, 25230, 25231, 25232, 25233, 25234, 25235, 25236, 25237, 25238, 25239, 25240, 25241, 25242) c6( ) c7(T, dD, ea, aD, ht, aA, bB, fd, eq, ai, ad, hX, iu, AW, hd, aE, ds, kH, aT, io, h, hu, hB, ae, aa, aEV, d, B, mR, iU, xR, aW, Y, fJ, az, bkf, ff, fe, lo, aF, Fq, Sm, pL, jT, en, bz, g, ik, bw, Qy, eU, bE, yH, jM, y, Pr, X, nQ); #c1(IL33) c2(NP_001186569) c3(interleukin-33 isoform 2) c4(77855) c5(77856, 77857, 77858, 77859, 77860, 77861) c6( ) c7(bM, EM, dD, aCA, fR, ea, yk, e, aw, l, v, j, eq, Jx, V, ai, aFu, BJ, wD, xy, aE, oz, bW, wb, lQ, ji, aOh, hH, af, aa, bRh, Nr, asp, lx, mR, iE, ff, cr, dN, po, lo, fe, Fq, bRj, en, BE, iV, bRi, adz, Nq, LB, cT, lt, aDu, aCT, NL, z, gw, at, aS, dy, fD, agl, hE, B, y); #c1(IL33) c2(NP_001186570) c3(interleukin-33 isoform 3) c4(77862) c5(77863, 77864, 77865, 77866, 77867, 77868) c6( ) c7(bM, EM, dD, aCA, fR, ea, yk, e, aw, l, v, j, eq, Jx, V, ai, aFu, eJ, wD, xy, aE, oz, bW, wb, lQ, ji, aOh, hH, af, aa, bRh, Nr, asp, lx, mR, iE, ff, cr, dN, po, lo, fe, Fq, bRj, en, eE, iV, bRi, adz, Nq, LB, cT, lt, aDu, aCT, NL, z, gw, at, aS, dy, fD, agl, hE, B, y); #c1(IL33) c2(NP_254274) c3(interleukin-33 isoform 1 precursor) c4(228669) c5(228670, 228671, 228672, 228673, 228674, 228675) c6( ) c7(bM, EM, dD, aCA, fR, ea, yk, e, aw, l, v, j, eq, Jx, V, ai, aFu, eJ, wD, xy, aE, oz, bW, wb, lL, ji, aOh, hH, af, aa, bRh, Nr, asp, lx, mR, iE, ff, cr, dN, po, lo, fe, Fq, bRj, en, eE, iV, bRi, adz, Nq, LB, cT, lt, aDu, aCT, NL, z, gw, at, aS, dy, fD, agl, hE, B, y); #c1(IL34) c2(XP_011521205) c3(interleukin-34 isoform X1) c4(316130) c5(316131, 316132, 316133, 316134, 316135, 316136, 316137, 316138, 316139, 316140) c6( ) c7(v, y, kl, ai, zr); #c1(IL36A) c2(NP_055255) c3(interleukin-36 alpha) c4(180073, 180074) c5(180075, 180076, 180077, 180078, 180079, 180080, 180081, 180082, 180083, 180084, 180085, 180086) c6( ) c7(yE, y, mR); #c1(IL36B) c2(NP_055253) c3(interleukin-36 beta isoform 1) c4(180059) c5(180060, 180061, 180062, 180063, 180064, 180065) c6( ) c7(y, mR); #c1(IL36B) c2(NP_775270) c3(interleukin-36 beta isoform 2) c4(249746) c5(249747, 249748, 249749, 249750, 249751, 249752) c6( ) c7(y, mR); #c1(IL36G) c2(NP_062564) c3(interleukin-36 gamma isoform 1) c4(201984, 201985) c5(201986, 201987, 201988, 201989, 201990, 201991, 201992, 201993, 201994, 201995, 201996, 201997) c6( ) c7(y, mR, eW); #c1(IL36RN) c2(NP_775262) c3(interleukin-36 receptor antagonist protein) c4(249691, 249692) c5(249693, 249694, 249695, 249696, 249697, 249698, 249699, 249700, 249701, 249702, 249703, 249704, 249705, 249706, 249707, 249708) c6( ) c7(mR, ac, Jr, atj, e, aw, cr, bRk, j, lQ, y, eu, wD); #c1(IL37) c2(NP_055254) c3(interleukin-37 isoform 1 precursor) c4(180066) c5(180067, 180068, 180069, 180070, 180071, 180072) c6( ) c7(oO, T, hF, q, ld, dm, hH, azU, mR, Pg, cr, eZ, fe, iV, eE, bz, Jr, vZ, dy, y, qR, B); #c1(IL37) c2(NP_775294) c3(interleukin-37 isoform 2) c4(249897) c5(249898, 249899, 249900, 249901, 249902, 249903) c6( ) c7(oO, T, hF, q, ld, dm, hH, azU, mR, Pg, cr, eZ, fe, iV, eE, bz, Jr, vZ, dy, y, qR, B); #c1(IL37) c2(NP_775295) c3(interleukin-37 isoform 3) c4(249904) c5(249905, 249906, 249907, 249908, 249909, 249910) c6( ) c7(oO, T, hF, q, ld, dm, hH, azU), mR, Pg, cr, eZ, fe, iV, eE, bz, Jr, vZ, dy, y, qR, B); #c1(IL37) c2(NP_775296) c3(interleukin-37 isoform 4) c4(249911) c5(249912, 249913, 249914, 249915, 249916, 249917) c6( ) c7(oO, T, hF, q, ld, dm, hH, azU, mR, Pg, cr, eZ, fe, iV, eE, bz, Jr, vZ, dy, y, qR, B); #c1(IL37) c2(NP_775297) c3(interleukin-37 isoform 5) c4(249918) c5(249919, 249920, 249921, 249922, 249923, 249924) c6( ) c7(oO, T, hF, q, ld, dm, hH, azU, mR, Pg, cr, eZ, fe, iV, BE, bz, Jr, vZ, dy, y, qR, B); #c1(IL3RA) c2(NP_001254642) c3(interleukin-3 receptor subunit alpha isoform 2 precursor) c4(94776, 94777) c5(94778, 94779, 94780, 94781, 94782, 94783, 94784, 94785, 94786, 94787, 94788, 94789) c6( ) c7(ck, x, sY, EW, fX, uC, vJ, zK, AH, hn, k, bt); #c1(IL3RA) c2(NP_002174) c3(interleukin-3 receptor subunit alpha isoform 1 precursor) c4(126294) c5(126295, 126296, 126297, 126298, 126299, 126300) c6( ) c7(ck, x, sY, EW, fX, uC, vJ, zK, AH, hn, k, bt); #c1(IL4) c2(NP_000580) c3(interleukin-4 isoform 1 precursor) c4(13149) c5(13150, 13151, 13152, 13153, 13154, 13155, 13156) c6( ) c7(gh, T, ea, fd, ci, bRn, aY, bv, xy, aE, fm, bRp, rw, dV, Ds, zy, hH, azh, cp, aa, li, az, ih, nY, bRm, zV, ba, fT, aZx, iA, nB, aCT, dc, cj, k, eg, fy, uL, aDi, qm, nD, aYZ, ht, aj, cd, zo, apG, ey, mR, sp, aRL, fe, CQ, adz, bbz, aDu, dB, ajd, abW, jv, aA, cz, q, zn, bIN, Oi, bPW, aAD, j, DX, eu, fO, bg, Ox, lo, Xk, pb, cT, pa, iM, ac, iQ, ig, bRs, aor, bt, aCA, wi, BD, tS, aPJ, iW, jh, dG, fu, aue, ad, wD, hX, oW, lQ, DI, lx, qZ, ff, Of, iV, bRq, bz, DF, Cq, anY, aDB, ME, lf, ra, ne, rk, oO, bM, xS, if, bB, cL, lN, bh, asr, Tz, ip, x, fS, azm, bc, Du, hl, kt, pD, as, aFU, BE, cm, bw, xD, ki, vA, mx, OT, hh, Vj, bBT, dD, yG, n, uE, zw, WB, brX, eV, cY, bFQ, xP, A, fJ, po, yq, amo, xe, jq, bJa, at, bPs, ut, eo, FL, B, dA, lH, eX, fF, v, Qo, V, ld, bRt, bPF, ae, aAi, jf, hM, ev, afS, acB, t, ck, ll, yH, vi, df, bQb, OR, wV, eq, bNM, hd, aop, afm, ds, aCJ, qh, hD, rN, JK, Zd, sj, d, je, bPY, wc, dN, kD, Z, dr, gk, dO, wz, y, kS, pC, aC, fR, al, lj, qj, lR, Qp, wa, bOm, AW, xE, bW, K, aZS, aDU, eL, aCM, axu, asD, mQ, aB, g, pL, uC, ou, aDs, uo, dW, Hb, m, aZN, sQ, sw, aw, fN, qy, BF, pM, eh, KH, cU, aDp, gi, Og, cM, bMc, kT, byl, xO, JM, Pr, bQZ, fM, fG, jx, hz, bJw, j, eA, J, o, er, XX, jZ, bRo, xR, Y, r, en, ik, bN, Qi, sn, aQd, hc, wR, Da, rK, dy, EY, fD, gT, LS, sY, bjX, id, aFe, Pt, em, bS, ah, hF, bRl, ase, dP, sN, aNp, hP, ji, adx, qE, vj, Sn, cr, VI, hp, Fq, BG, jg, bNW, Nq, TA, Lj, asq, aFb, eB, bC, hE, xg, bRu, G, eb, Jx, mF, fw, aR, pk, azx, aW, OZ, hJ, eM, iT, Jp, zv, fr, ct, qN, TI, ai, eW, cc, h, dL, bxg, Nr, asp, bRr, aq, kp, bRv, gG, kk, op, Wt, asE, tk, NL, hk, uz, va, aDE, bni, bL, Dw, ka, eR, yk, C, ady, ga, aCY, mP, aT, adV, aSX, an, af, ls, Uf, iU, oV, Ph, f, ajl, nM, vZ, lV, CZ, ij, qX, qb, aD, vr, jE, mC, kL, qC, av, l, F, yF, uQ, bwk, yx, eZ, aFr, aF, ek, LB, es, eU, zr, bE, z, XO, bPJ, X); #c1(IL4) c2(NP_758858) c3(interleukin-4 isoform 2 precursor) c4(249286) c5(249287, 249288, 249289, 249290, 249291, 249292, 249293) c6( ) c7(gh, T, ea, fd, ci, bRn, aY, bv, xy, aE, fm, bRp, rw, dV, Ds, zy, hH, azh, cp, aa, li, az, ih, nY, bRm, zV, ba, fT, aZx, iA, nB, aCT, dc, cj, k, eg, fy, uL, aDi, qm, nD, aYZ, ht, aj, cd, zo, apG, ey, mR, sp, aRL, fe, CQ, adz, bbz, aDu, dB, ajd, abW, jv, aA, cz, q, zn, bIN, Di, bPW, aAD, j, DX, eu, fO, bg, Ox, lo, Xk, pb, cT, pa, iM, ac, iQ, ig, bRs, aor, bt, aCA, wi, BD, tS, aPJ, iW, jh, dG, fu, aue, ad, wD, hX, oW, lQ, DI, lx, qZ, ff, Of, iV, bRq, bz, DF, Cq, anY, aDB, ME, lf, ra, ne, rk, oO, bM, xS, if, bB, cL, lN, bh, asr, Tz, ip, x, fS, azm, bc, Du, hl, kt, pD, as, aFU, eE, cm, bw, xD, ki, vA, mx, OT, hh, Vj, bBT, dD, yG, n, uE, zw, WB, brX, eV, cY, bFQ, xP, A, fJ, po, yq, amo, xe, jq, bJa, at, bPs, ut, eo, fL, B, dA, lH, ex, fF, v, Qo, V, ld, bRt, bPF, ae, aAi, jf, hM, ev, afS, acB, t, ck, ll, yH, vi, df, bQb, OR, wV, eq, bNM, hd, aop, afm, ds, aCJ, qh, hD, rN, JK, Zd, sj, d, je, bPY, wc, dN, kD, Z, dr, gk, dO, wz, y, kS, pC, aC, fR, al, lj, qj, lR, Qp, wa, bOm, AW, xE, bW, K, aZS, aDU, eL, aCM, axu, asD, mQ, aB, g, pL, uC, ou, aDs, uo, dW, Hb, m, aZN, sQ, sw, aw, fN, qy, BF, pM, eh, kH, cU, aDp, gi, Og, cM, bMc, kT, byl, xO, JM, Pr, bQZ, fM, fG, jx, hz, bJw, j, eA, J, o, er, XX, jZ, bRo, xR, Y, r, en, ik, bN, Qi, sn, and, hc, wR, Da, rK, dy, EY, fD, gT, LS, sY, bjX, id, aFe, Pt, em, bS, ah, hF, bRl, ase, dP, sN, aNp, hP, ji, adx, qE, vj, Sn, cr, VI, hp, Fq, BG, jg, bNW, Nq, TA, Lj, asq, aFb, eB, bC, hE, xg, bRu, G, eb, Jx, mF, fw, aR, pk, azx, aW, OZ, hJ, eM, iT, Jp, zv, fr, ct, qN, TI, ai, eW, cc, h, dL, bxg, Nr, asp, bRr, aq, kp, bRv, gG, kk, op, Wt, asE, tk, NL, hk, uz, va, aDE, bni, bL, Dw, ka, eR, yk, C, ady, ga, aCY, mP, aT, adV, aSX, an, af, ls, Uf, iU, oV, Ph, f, ajl, nM, vZ, lV, CZ, ij, qX, qb, aD, vr, jE, mC, kL, qC, av, l, F, yF, uQ, bwk, yx, eZ, aFr, af, ek, LB, es, eU, zr, bE, z, XO, bPJ, X); #c1(IL411) c2(NP_001244947) c3(L-amino-acid oxidase isoform 2 precursor) c4(92385, 92386, 92387, 92388, 92389, 92390) c5(92391, 92392, 92393, 92394, 92395, 92396, 92397, 92398, 92399, 92400, 92401, 92402, 92403, 92404, 92405, 92406) c6( ) c7(bNX, fM, ib, Y, ae, ad, uQ); #c1(IL411) c2(NP_690863) c3(L-amino-acid oxidase isoform 1 precursor) c4(244998, 244999, 245000, 245001, 245002, 245003, 245004, 245005, 245006, 245007, 245008, 245009, 245010, 245011) c5( ) c6( ) c7(bNX, fM, ib, Y, ae, ad, uQ); #c1(IL4R) c2(NP_001244336) c3(interleukin-4 receptor subunit alpha isoform c) c4(92196) c5(92197, 92198, 92199, 92200, 92201, 92202) c6( ) c7(bM, T, aC, xS, fR, if, al, fd, lj, cL, lN, ci, aR, aE, aLI, bRx, bW, fS, dV, Ds, aW, axu, az, mQ, kt, g, pL, eE, uC, QA, fT, xK, cj, uo, k, eg, uL, FW, sQ, aw, aj, fr, pM, ai, ql, kH, cU, h, eV, dL, aDp, A, sp, aCx, fe, po, Og, CQ, xe, cM, adz, kT, RB, dB, hk, at, ut, gg, bL, Dw, oe, jx, eX, rf, aA, v, j, C, Do, V, q, eA, ga, bIN, aT, ae, af, ne, bA, eu, Uf, Y, ayU, bg, r, en, pb, cT, rR, iQ, ac, Ty, ll, dy, blu, sY, bRw, Ke, qb, aD, iW, jh, auq, pl, ah, jE, ase, wD, hX, F, hD, rN, JK, lQ, bGD, sj, d, qZ, vj, ff, Of, cr, eZ, aFr, bpA, aF, hp, iV, LB, es, D, z, wz, sG, dX, aS, y, X); #c1(IL4R) c2(XP_011544129) c3(interleukin-4 receptor subunit alpha isoform X1) c4(343278, 343279, 343280) c5(343281, 343282, 343283, 343284, 343285, 343286, 343287, 343288, 343289, 343290, 343291, 343292, 343293, 343294, 343295, 343296, 343297, 343298, 343299, 343300, 343301, 343302, 343303, 343304, 343305, 343306) c6( ) c7(bM, T, aC, xS, FR, if, al, fd, lj, cL, lN, ci, aR, aE, aLI, bRx, bW, fS, dV, Ds, aW, axu, az, md, kt, g, pL, eE, uC, QA, fT, xK, cj, uo, k, eg, uL, FW, sQ, aw, aj, fr, pM, ai, ql, kH, cU, h, eV, dL, aDp, A, sp, aCx, fe, po, Og, CQ, xe, cM, adz, kT, RB, dB, hk, at, ut, gg, bL, Dw, oe, jx, eX, rf, aA, v, j, C, Do, V, q, eA, ga, bIN, aT, ae, af, ne, bA, eu, Uf, Y, ayU, bg, r, en, pb, cT, rR, iQ, ac, Ty, ll, dy, blu, sY, bRw, Ke, qb, aD, iW, jh, auq, pl, ah, jE, ase, wD, hX, F, hD, rN, JK, lQ, bGD, sj, d, qZ, vj, ff, Of, cr, eZ, aFr, bpA, aF, hp, iV, LB, es, D, z, wz, sG, dX, aS, y, X); #c1(IL5) c2(XP_006714664) c3(interleukin-5 isoform X2) c4(287980) c5(287981) c6( ) c7(bM, T, FR, Js, bRC, ea, atj, e, fd, bPw, bh, Jx, AW, bJG, Ds, K, azx, eL, bc, bNG, aa, axu, Du, nY, g, eE, bw, uC, zV, fT, iT, aCT, JA, oy, cj, uo, aZo, Dv, eg, aSs, LT, bBT, aZt, aiu, dD, Ht, akc, s, bRz, bRB, fN, cQ, ct, uE, pM, ju, kH, CU, Ne, h, dL, ey, eV, aDp, Nr, asp, sp, aRL, lb, fJ, fe, aq, po, kk, Wt, aDu, JM, ut, FL, WL, B, Jo, bL, Dw, jv, fM, bRA, dA, fG, Ju, eX, aA, cz, hz, aCY, blC, mP, apJ, Nw, aT, bPF, ae, tn, iU, xR, Y, lC, lo, Ph, en, bN, lY, Cv, bnc, cT, ck, iM, iQ, ig, of, dT, fD, bt, vi, sY, bjx, LX, iW, df, jh, bS, eq, bRy, hF, ase, ad, F, afm, ds, hD, aNp, lQ, yF, uQ, je, lx, bPY, ff, eZ, dN, hp, iV, Z, DF, Nq, TA, eU, z, aDB, aKC, azM, i, hE, bRu, kd, y); #c1(IL5) c2(XP_011541676) c3(interleukin-5 isoform X1) c4( ) c5(340213, 340214, 340215, 340216, 340217) c6( ) c7(bM, T, fR, Js, bRC, ea, atj, e, fd, bPw, bh, Jx, AW, bJG, Ds, K, azx, eL, bc, bNG, aa, axu, Du, nY, g, eE, bw, uC, zV, fT, iT, aCT, JA, oy, cj, uo, aZo, Dv, eg, aSs, LT, bBT, aZt, aiu, dD, Ht, akc, sQ, bRz, bRB, fN, cQ, ct, uE, pM, ju, KH, cU, Ne, h, dL, ey, eV, aDp, Nr, asp, sp, aRL, lb, fJ, fe, aq, po, kk, Wt, aDu, JM, ut, FL, WL, B, Jo, bL, Dw, jv, fM, bRA, dA, fG, Ju, eX, aA, cz, hz, aCY, blC, mP, apJ, Nw, aT, bPF, ae, tn, iU, xR, Y, lC, lo, Ph, en, bN, lY, Cv, bnc, cT, ck, iM, iQ, ig, of, dT, fD, bt, vi, sY, bjX, LX, iW, df, jh, bS, eq, bRy, hF, ase, ad, F, afm, ds, hD, aNp, lQ, yF, uQ, je, lx, bPY, ff, eZ, dN, hp, iV, Z, DF, Nq, TA, eU, z, aDB, akC, azM, i, hE, bRu, kd, y); #c1(IL5RA) c2(NP_000555) c3(interleukin-5 receptor subunit alpha isoform 1 precursor) c4(12648) c5(12649, 12650, 12651, 12652, 12653, 12654, 12655, 12656, 12657, 12658) c6( ) c7(bL, bM, sY, Dw, j, V, AW, apJ, Ds, dL, JK, ey, eL, eu, Nr, iU, sp, mQ, Xm, hp, eE, uC, cM, Nq, kT, ac, cj, fD, y); #c1(IL5RA) c2(NP_001230028) c3(interleukin-5 receptor subunit alpha isoform 4 precursor) c4(85832) c5(85833, 85834, 85835, 85836, 85837, 85838) c6( ) c7(bL, bM, sY, Dw, j, V, AW, apJ, Ds, dL, JK, ey, eL, eu, Nr, iU, sp, mQ, Xm, hp, eE, uC, cM, Nq, kT, ac, cj, fD, y); #c1(IL5RA) c2(NP_783854) c3(interleukin-5 receptor subunit alpha isoform 2 precursor) c4(251731, 251732, 251733) c5(251734, 251735, 251736, 251737, 251738, 251739, 251740, 251741, 251742, 251743) c6( ) c7(bL, bM, sY, Dw, j, V, AW, apJ, Ds, dL, JK, ey, eL, eu, Nr, iU, sp, m, Xm, hp, eE, uC, cM, Nq, kT, ac, cj, fD, y); #c1(IL5RA) c2(NP_783855) c3(interleukin-5 receptor subunit alpha isoform 3 precursor) c4(251744, 251745, 251746) c5(251747, 251748, 251749, 251750, 251751, 251752, 251753, 251754, 251755, 251756) c6( ) c7(bL, bM, sY, Dw, j, V, AW, apJ, Ds, dl, JK, ey, eL, eu, Nr, iU, sp, mQ, Xm, hp, eE, uC, cM, Nq, kT, ac, cj, fD, y); #c1(IL5RA) c2(XP_011531980) c3(interleukin-5 receptor subunit alpha isoform X1) c4(327983) c5(327984, 327985, 327986, 327987, 327988, 327989) c6( ) c7(bL, bM, sY, Dw, j, V, AW, apJ, Ds, dL, JK, ey, eL, eu, Nr, iU, sp, mQ, Xm, hp, eE, uC, cM, Nq, kT, ac, cj, fD, y); #c1(IL5RA) c2(XP_011531981) c3(interleukin-5 receptor subunit alpha isoform X2) c4( ) c5(327990, 327991, 327992, 327993, 327994, 327995, 327996, 327997, 327998, 327999, 328000, 328001, 328002, 328003, 328004, 328005, 328006, 328007, 328008, 328009, 328010, 328011, 328012, 328013) c6( ) c7(bL, bM, sY, Dw, j, V, AW, apJ, Ds, dL, JK, ey, eL, eu, Nr, iU, sp, mQ, Xm, hp, eE, uC, cM, Nq, kT, ac, cj, fD, y); #c1(IL6) c2(NP_000591) c3(interleukin-6 precursor) c4(13368, 13369) c5(13370, 13371, 13372, 13373, 13374, 13375, 13376, 13377, 13378, 13379, 13380, 13381, 13382, 13383, 13384, 13385, 13386, 13387, 13388, 13389, 13390, 13391) c6(13392, 13393, 13394, 13395, 13396, 13397) c7(uB, gh, T, os, ea, bO, fd, Vd, ci, aY, bv, bQB, aE, rw, dV, Vv, alX, Ds, Bi, hu, zy, hH, azh, cp, VF, aa, ly, CG, yo, az, nY, vs, zC, axp, lk, akw, baR, GR, axG, zV, ba, fT, iA, Jr, nB, dc, wE, cj, Dt, k, azg, aEw, sr, nD, dt, FG, ht, aj, bPL, qW, cd, br, zo, td, adf, aEB, lG, bxi, aNj, ey, bl, dd, nP, mR, fe, aVD, CA, pm, adz, hq, vU, iO, aBs, ok, RB, dB, bPc, eD, bOj, Ei, E, avr, aZD, bQi, abW, apE, DM, jv, aZy, DD, fZ, aA, cz, bX, yV, lu, wy, q, zn, aEE, aCX, yu, bPW, cy, aLM, aAD, jj, ch, dl, eu, bA, le, bf, nO, fO, lC, bg, bsl, aMA, aFC, lo, bRQ, cl, GF, pb, cT, rR, acW, tW, ac, pa, ig, iM, iQ, mG, aXB, bRN, hr, MB, bRJ, bt, Ze, blz, ao, On, gV, Ve, wi, aag, arS, iW, uG, jN, rl, jh, aHv, mw, oZ, dG, uu, fu, ad, wD, aoH, hX, bRP, yt, bRE, oW, iY, lQ, bPM, WZ, MG, DI, aFP, lx, azS, cH, PE, cW, ff, Of, Mz, tv, dq, EZ, iV, aFd, bz, baa, OG, amS, xZ, ag, dX, lC, lf, bRF, ne, iu, ra, bsJ, oO, bM, lD, qo, vS, bQl, aiH, Tc, if, e, bB, cL, ur, lN, bh, ajC, Tz, ip, x, vV, Gb, ja, bc, bQs, xQ, eG, up, Jl, fc, hl, kt, pD, as, Eg, BE, eF, cm, bw, qJ, eY, lt, bQl, xD, ki, mx, vy, aGN, wx, Vj, aiu, dD, gU, lT, zt, n, nA, aCZ, uE, Mo, jz, no, pe, ec, zw, brX, gd, bsg, bRM, eV, hn, bQx, bRH, cY, A, RT, aix, hT, fJ, bQq, yq, amo, vo, xe, Sc, bP, cA, PI, hL, at, bPs, bjK, ut, pg, fL, B, NQ, AS, dA, mr, wL, Lx, eX, fF, GC, vv, aNG, v, bcE, Qo, V, akG, eJ, aPW, asG, di, bQG, DK, rS, sl, ld, aWJ, XZ, bau, ae, to, blm, aFs, hM, jf, Sh, bRL, zH, aOK, vR, ev, DN, akB, aom, aDY, LV, t, acp, vk, bRK, ck, amv, kP, ll, wO, gX, hO, ro, oX, yH, byK, zu, vi, dM, amQ, aBz, OR, wV, eq, adg, qi, hd, aop, py, afm, Db, ds, Hk, LA, hD, rN, JK, nJ, bOu, sj, atX, d, je, wc, NH, aFt, rM, dN, kD, anW, Z, aGj, HI, Hv, dr, D, gk, KD, yE, dO, ll, wz, gZ, aef, MJ, Hs, y, pC, aC, UH, fR, vf, atj, bll, al, axk, gf, qj, lR, wa, xE, u, aEX, Xc, bW, pX, M, rB, K, aZS, wt, DY, dF, eL, bNG, mH, aEV, ago, HI, bOh, lW, axu, asD, mQ, LZ, hN, FU, aB, Ku, g, uC, DA, Uk, bRl, aik, bRO, xK, aFO, aDs, uy, aer, U, uo, dW, Hb, m, P, NK, aFQ, sl, sw, aw, NT, fN, To, BH, BF, aCQ, eh, ft, Nn, KH, Lv, CU, Ne, Jn, wb, uV, ZN, Ha, tx, zq, Ni, Og, jT, cM, bRD, auC, Zj, ar, kT, GG, W, xO, wk, bo, mg, aiS, gg, Pr, it, L, ayV, fM, wj, fG, oe, jx, Nt, wo, zN, hz, aSE, j, eA, J, o, er, hb, nX, jZ, dn, aCV, yg, qG, bq, bPQ, aRR, azU, kl, PK, Y, RF, Pg, r, wl, tu, en, wA, iK, bN, QG, sn, aQd, hc, of, dh, tM, wR, cS, dy, fD, Kg, LS, sY, kh, bml, aFe, bxK, am, id, aZ, em, bs, bPD, auq, bS, ah, pl, bKZ, hF, azk, atK, iJ, Ho, dP, fU, sN, aZW, ji, bNL, NM, qE, uX, lR, anX, vj, xs, eH, cr, aZM, hp, BG, iR, Bu, KM, bNW, qa, dR, TA, Bv, Lj, HP, afb, sG, aEs, aCl, cP, ei, hE, pf, eb, aTy, aR, aEQ, dY, YX, iX, Ou, qM, bU, dQ, H, eN, bRR, hm, tw, DA, EX, bvR, LC, bRG, aW, bNq, Rr, aXc, aOW, aAu, brr, hJ, eM, iT, Jp, uJ, gn, R, xh, qR, cZ, JC, My, Mi, ct, qN, fr, kO, aPb, ai, rc, eW, aDF, ke, co, cc, ql, kl, ju, zf, h, jy, dL, ux, pj, vx, afu, asp, aiB, alt, aq, kp, kk, wU, op, lx, bJ, oU, Wt, asE, NL, hk, vb, bRS, bm, hR, uz, te, aDE, amb, bni, cn, aMk, bL, Dw, eR, dk, uv, DV, sM, cR, hy, C, aql, ady, ga, aCY, mP, qp, afQ, aT, an, af, ak, Uf, s, iU, aZB, oV, la, aNi, akY, aDm, aYp, bQ, f, Vq, lY, wf, Ta, sS, byx, wg, nM, Zg, alt, AH, fP, wq, adR, dT, mf, hS, Kd, Ke, ZI, aD, ha, vr, oH, jE, mC, KL, av, bPu, l, F, MS, nH, xl, oz, ib, NS, alg, Os, vw, jo, yF, uQ, Di, qt, aFE, aGS, eZ, yy, gl, aFr, xv, aF, gu, byM, uF, eK, LB, es, pc, zr, axs, bE, pE, atM, dJ, uR, z, aS, aGg, tr, bPJ, pk, X, us); #c1(IL6) c2(XP_005249802) c3(interleukin-6 isoform X2) c4(269855, 269856, 269857) c5(269858, 269859, 269860, 269861, 269862, 269863, 269864, 269865, 269866, 269867, 269868, 269869, 269870, 269871, 269872, 269873, 269874, 269875, 269876, 269877, 269878, 269879, 269880, 269881) c6(269882, 269883, 269884, 269885, 269886, 269887) c7(uB, gh, T, os, ea, bO, fd, Vd, ci, aY, bv, bQB, aE, rw, dV, Vv, alX, Ds, Bi, hu, zy, hH, azh, cp, VF, aa, ly, CG, yo, az, nY, vs, zC, axp, lk, akw, baR, GR, axG, zV, ba, fT, iA, Jr, nB, dc, wE, cj, Dt, k, azg, aEw, sr, nD, dt, FG, ht, aj, bPL, qW, cd, br, zo, td, adf, aEB, lG, bxi, aNj, ey, bl, dd, nP, mR, fe, aVD, CQ, pm, adz, hq, vU, iO, aBs, ok, RB, dB, bPc, eD, bOj, Ei, E, avr, aZD, bQi, abW, apE, DM, jv, aZy, DD, fZ, aA, cz, bX, yV, lu, wy, q, zn, aEE, aCX, yu, bPW, cy, aLM, aAD, j, ch, dl, eu, bA, le, bf, nO, fO, lC, bg, bsl, aMA, aFC, lo, bRQ, cl, GF, pb, cT, rR, acW, tW, ac, pa, ig, iM, iQ, mG, aXB, bRN, hr, MB, bRJ, bt, Ze, blz, ao, On, gV, Ve, wi, aag, arS, iW, uG, jN, rl, jh, aHv, mw, oZ, dG, uu, fu, ad, wD, aoH, hX, bRP, yt, bRE, oW, iY, lQ, bPM, WZ, MG, DI, aFP, lx, azS, cH, PE, cW, ff, Of, Mz, tv, dq, EZ, iV, aFd, bz, baa, OG, amS, xZ, ag, dX, lC, lf, bRF, ne, iu, ra, bsJ, oO, bM, lD, qo, vS, bQl, aiH, Tc, if, e, bB, cL, ur, lN, bh, ajC, Tz, ip, x, vV, Gb, ja, bc, bQs, xQ, eG, up, JI, fc, hl, kt, pD, as, Eg, eE, eF, cm, bw, qJ, eY, lt, bQl, xD, ki, mx, vy, aGN, wx, Vj, aiu, dD, gU, lT, zt, n, nA, aCZ, uE, Mo, jz, no, pe, ec, zw, brX, gd, bsg, bRM, eV, hn, bQx, bRH, cY, A, RT, aix, hT, fJ, bQq, yq, amo, vo, xe, Sc, bP, cA, PI, hL, at, bPs, bjk, ut, pg, fL, B, NO, AS, dA, mr, wL, Lx, eX, fF, GC, vv, aNG, v, bcE, Do, V, akG, eJ, aPW, asG, di, bQG, DK, rS, sl, ld, aWJ, XZ, bau, ae, to, blm, aFs, hM, jf, Sh, bRL, zH, aOK, vR, ev, DN, akB, aom, aDY, LV, t, acp, vk, bRK, ck, amv, kP, ll, wO, gX, hO, ro, oX, yH, byK, zu, vi, dM, amd, aBz, OR, wV, eq, adg, qi, hd, aop, py, afm, Db, ds, Hk, LA, hD, rN, JK, nJ, bOu, sj, atX, d, je, wc, NH, aFt, rM, dN, kD, anW, Z, aGj, HI, Hv, dr, D, gk, KD, yE, dO, ll, wz, gZ, aef, MJ, Hs, y, pC, aC, UH, fR, vf, atj, bll, al, axk, gf, qj, lR, wa, xE, u, aEX, Xc, bW, pX, M, rB, K, aZS, wt, DY, dF, eL, bNG, mH, aEV, ago, HI, bOh, lW, axu, asD, mQ, LZ, hN, FU, aB, Ku, g, uC, QA, Uk, bRl, aik, bRO, xK, aFO, aDs, uy, aer, U, uo, dW, Hb, m, P, NK, aFQ, sQ, sw, aw, NT, fN, To, BH, BF, aCQ, eh, ft, Nn, KH, Lv, cU, Ne, Jn, wb, uV, ZN, Ha, tx, zq, Ni, Og, jT, cM, bRD, auC, Zj, ar, kT, GG, W, xO, wk, bo, mg, aiS, gg, Pr, it, L, ayV, fM, wj, fG, oe, jx, Nt, wo, zN, hz, aSE, j, eA, J, o, er, hb, nX, jZ, dn, aCV, yg, qG, bq, bPQ, aRR, azU, kl, PK, Y, RF, Pg, r, wl, tu, en, wA, iK, bN, QG, sn, and, hc, of, dh, tM, wR, cS, dy, fD, Kg, LS, sY, kh, bmQ, aFe, bxK, am, id, aZ, em, bs, bPD, auq, bS, ah, pl, bKZ, hF, azk, atK, iJ, Ho, dP, fU, sN, aZW, ji, bNL, NM, qE, uX, lR, anX, vj, xs, eH, cr, aZM, hp, BG, iR, Bu, KM, bNW, qa, dR, TA, Bv, Lj, HP, aFb, sG, aEs, aCl, cP, ei, hE, pf, eb, aTy, aR, aEQ, dY, YX, iX, Ou, qM, bU, dQ, H, eN, bRR, hm, tw, DA, EX, bvR, LC, bRG, aW, bNq, Rr, aXc, aDW, aAu, brr, hJ, eM, iT, Jp, uJ, gn, R, xh, qR, cZ, JC, My, Mi, ct, qN, fr, kO, aPb, ai, rc, eW, aDF, ke, co, cc, ql, kl, ju, zf, h, jy, dL, ux, pj, vx, afu, asp, aiB, alt, aq, kp, kk, wU, op, lx, bJ, ou, Wt, asE, NL, hk, vb, bRS, bm, hR, uz, te, aDE, amb, bni, cn, aMk, bL, Dw, eR, dk, uv, DV, sM, cR, hy, C, aql, ady, ga, aCY, mP, qp, afQ, aT, an, af, ak, Uf, s, iU, aZB, oV, la, aNi, akY, aDm, aYp, bQ, f, Vq, lY, wf, Ta, sS, byx, wg, nM, Zg, alt, AH, fP, wq, adR, dT, mf, hS, Kd, Ke, ZI, aD, ha, vr, oH, jE, mC, KL, av, bPu, l, F, MS, nH, xl, oz, ib, NS, alg, Os, vw, jo, yF, uQ, Di, qt, aFE, aGS, eZ, yy, gl, aFr, xv, aF, gu, byM, uF, ek, LB, es, pc, zr, axs, bE, pE, atM, dJ, uR, z, aS, aGg, tr, bPJ, pk, X, us); #c1(ILGR) c2(NP_000556) c3(interleukin-6 receptor subunit alpha isoform 1 precursor) c4(12659) c5(12660, 12661, 12662, 12663, 12664, 12665) c6( ) c7(bM, T, aC, fR, ea, fd, ci, Ds, rB, hH, eL, yo, JI, aW, aDn, eF, cm, uC, bw, eM, cj, uo, aYS, m, azg, dD, sQ, sw, My, ht, aw, aj, cd, zo, pe, h, dL, bl, uV, fe, aq, CQ, hk, bm, at, uz, ut, hR, agy, B, bL, Dw, mr, fG, aA, v, j, V, eA, J, aT, cy, af, bA, Y, en, t, ck, Ta, iM, ac, np, sY, Ve, em, jh, dG, eq, ad, hX, iY, lQ, je, ff, eH, eZ, af, hp, bz, LB, aGg, ei, y, Tc); #c1(ILGR) c2(NP_852004) c3(interleukin-6 receptor subunit alpha isoform 2 precursor) c4(254799) c5(254800, 254801, 254802, 254803, 254804, 254805) c6( ) c7(bM, T, aC, fR, ea, fd, ci, Ds, rB, hH, eL, yo, JI, aW, aDn, eF, cm, uC, bw, eM, cj, uo, aYS, m, azg, dD, sQ, sw, My, ht, aw, aj, cd, zo, pe, h, dL, bl, uV, fe, aq, CQ, hk, bm, at, uz, ut, hR, agy, B, bL, Dw, mr, fG, aA, v, j, V, eA, J, aT, cy, af, bA, Y, en, t, ck, Ta, iM, ac, np, sY, Ve, em, jh, dG, eq, ad, hX, iY, lQ, je, ff, eH, eZ, af, hp, bz, LB, aGg, ei, y, Tc); #c1(IL6ST) c2(NP_001177910) c3(interleukin-6 receptor subunit beta isoform 3 precursor) c4(73125, 73126) c5(73127, 73128, 73129, 73130, 73131, 73132, 73133) c6( ) c7(bL, T, dD, lT, aD, aw, aA, cz, fd, v, j, ci, bX, eq, qN, zP, hF, qW, cd, ai, eA, x, jz, aE, pe, Xc, rw, Yo, aT, h, ji, K, uV, ae, af, eu, jf, yo, zQ, s, aW, Y, ff, aGS, hl, mQ, fe, aq, lo, aF, yq, en, g, t, bmj, uC, ck, LB, pc, dB, afc, mx, cj, hR, uz, m, k, fD, hh, ei, bt, cB, B, y); #c1(IL6ST) c2(NP_002175) c3(interleukin-6 receptor subunit beta isoform 1 precursor) c4(126301) c5(126302, 126303, 126304, 126305, 126306, 126307, 126308, 126309, 126310, 126311, 126312, 126313) c6( ) c7(bL, T, dD, lT, aD, aw, aA, cz, fd, v, j, ci, bX, eq, qN, zP, hF, qW, cd, ai, eA, x, jz, aE, pe, Xc, rw, Yo, aT, h, ji, K, uV, ae, af, eu, jf, yo, zQ, s, aW, Y, ff, aGS, hl, mQ, fe, aq, lo, aF, yq, en, g, t, bmj, uC, ck, LB, pc, dB, afc, mx, cj, hR, uz, m, k, fD, hh, ei, bt, cB, B, y); #c1(IL6ST) c2(NP_786943) c3(interleukin-6 receptor subunit beta isoform 2 precursor) c4(251939) c5(251940, 251941, 251942, 251943, 251944, 251945, 251946, 251947, 251948, 251949, 251950, 251951, 251952, 251953, 251954, 251955, 251956) c6( ) c7(bL, T, dD, lT, aD, aw, aA, cz, fd, v, j, ci, bX, eq, qN, zP, hF, qW, cd, ai, eA, x, jz, aE, pe, Xc, rw, Yo, aT, h, ji, K, uV, ae, af, eu, jf, yo, zQ, s, aW, Y, ff, aGS, hl, mQ, fe, aq, lo, aF, yq, en, g, t, bmj, uC, ck, LB, pc, dB, afc, mx, cj, hR, uz, m, k, fD, hh, ei, bt, cB, B, y); #c1(IL7) c2(NP_001186815) c3(interleukin-7 isoform 2 precursor) c4(78625, 78626) c5(78627, 78628, 78629, 78630, 78631, 78632, 78633, 78634, 78635, 78636, 78637, 78638, 78639, 78640, 78641, 78642, 78643, 78644) c6(78645, 78646, 78647, 78648) c7(bM, T, fR, ea, fd, x, rw, Co, Ds, Us, aW, apm, pL, eE, cm, ou, eM, cj, k, m, dD, fN, aj, fr, ai, bRT, Mo, cc, KH, h, ox, OM, A, RT, CM, fe, CQ, Wt, eo, Dw, dA, fG, lH, j, C, V, q, J, eA, oT, rS, al, ae, hM, xR, Y, r, t, f, dS, ck, iM, ac, iQ, yH, fD, bt, aJc, jh, eq, EA, ad, hX, av, F, ds, lQ, uQ, je, aF, jg, iV, bz, eU, zr, y); #c1(IL7) c2(NP_001186817) c3(interleukin-7 isoform 4 precursor) c4(78649) c5(78650, 78651, 78652, 78653, 78654, 78655, 78656, 78657, 78658, 78659, 78660, 78661, 78662, 78663, 78664, 78665, 78666, 78667, 78668, 78669, 78670, 78671, 78672, 78673, 78674, 78675, 78676, 78677, 78678, 78679, 78680) c6(78681, 78682, 78683, 78684, 78685) c7(bM, T, FR, ea, fd, x, rw, Co, Ds, Us, aW, apm, pL, eE, cm, ou, eM, cj, k, m, dD, fN, aj, fr, ai, bRT, Mo, cc, KH, h, ox, OM, A, RT, CM, fe, CQ, Wt, eo, Dw, dA, fG, lH, j, C, V, q, J, eA, oT, rS, aT, ae, hM, xR, Y, r, t, f, dS, ck, iM, ac, iQ, yH, fD, bt, aJc, jh, eq, EA, ad, hX, av, F, ds, lQ, uQ, je, aF, jg, iV, bz, eU, zr, y); #c1(IL7) c2(XP_011515823) c3(interleukin-7 isoform X1) c4(308631, 308632) c5(308633, 308634, 308635, 308636, 308637, 308638, 308639, 308640, 308641, 308642, 308643, 308644, 308645, 308646, 308647, 308648, 308649, 308650, 308651, 308652, 308653, 308654, 308655, 308656, 308657, 308658, 308659, 308660, 308661, 308662, 308663, 308664, 308665, 308666, 308667, 308668, 308669, 308670, 308671, 308672, 308673, 308674, 308675, 308676, 308677, 308678, 308679, 308680, 308681, 308682, 308683, 308684, 308685, 308686, 308687, 308688, 308689, 308690, 308691, 308692, 308693, 308694, 308695, 308696, 308697, 308698, 308699, 308700, 308701, 308702, 308703, 308704, 308705, 308706, 308707, 308708, 308709, 308710, 308711, 308712, 308713, 308714, 308715, 308716, 308717, 308718, 308719, 308720, 308721, 308722, 308723, 308724, 308725, 308726, 308727, 308728, 308729, 308730, 308731, 308732, 308733, 308734, 308735, 308736, 308737, 308738, 308739, 308740, 308741, 308742, 308743, 308744, 308745, 308746, 308747, 308748, 308749, 308750, 308751, 308752, 308753, 308754, 308755, 308756, 308757, 308758, 308759, 308760, 308761, 308762, 308763, 308764, 308765, 308766, 308767, 308768, 308769, 308770, 308771, 308772, 308773, 308774, 308775, 308776, 308777, 308778, 308779, 308780) c6(308781, 308782, 308783) c7(bM, T, FR, ea, fd, x, rw, Co, Ds, Us, aW, apm, pL, eE, cm, ou, eM, cj, k, m, dD, fN, aj, fr, ai, bRT, Mo, cc, kH, h, ox, OM, A, RT, CM, fe, CQ, Wt, eo, Dw, dA, fG, lH, j, C, V, q, J, eA, oT, rS, aT, ae, hM, xR, Y, r, t, f, dS, ck, iM, ac, iQ, yH, fD, bt, aJc, jh, eq, EA, ad, hX, av, F, ds, lQ, uQ, je, aF, jg, iV, bz, eU, zr, y); #c1(IL7) c2(XP_011515824) c3(interleukin-7 isoform X2) c4(308784) c5( ) c6( ) c7(bM, T, fR, ea, fd, x, rw, Co, Ds, Us, aW, apm, pL, eE, cm, ou, eM, cj, k, m, dD, fN, aj, fr, ai, bRT, Mo, cc, KH, h, ox, OM, A, RT, CM, fe, CQ, Wt, eo, Dw, dA, fG, lH, j, C, V, q, J, eA, oT, rS, aT, ae, hM, xR, Y, r, t, f, dS, ck, iM, ac, iQ, yH, fD, bt, aJc, jh, eq, EA, ad, hX, av, F, ds, lQ, uQ, je, aF, jg, iV, bz, eU, zr, y); #c1(IL7) c2(XP_011515825) c3(interleukin-7 isoform X3) c4(308785) c5(308786) c6( ) c7(bM, T, FR, ea, fd, x, rw, Co, Ds, Us, aW, apm, pL, eE, cm, ou, eM, cj, k, m, dD, fN, aj, fr, ai, bRT, Mo, cc, kH, h, ox, OM, A, RT, CM, fe, CQ, Wt, eo, Dw, dA, fG, lH, j, C, V, q, J, eA, oT, rS, aT, ae, hM, xR, Y, r, t, f, dS, ck, iM, ac, iQ, yH, fD, bt, aJc, jh, eq, EA, ad, hX, av, F, ds, lQ, uQ, je, aF, jg, iV, bz, eU, zr, y); #c1(IL7R) c2(NP_002176) c3(interleukin-7 receptor subunit alpha precursor) c4(126314, 126315, 126316) c5(126317, 126318, 126319, 126320, 126321, 126322, (26323) c6( ) c7(Kd, T, bM, dA, Ke, iW, jx, aA, aGx, cL, v, ci, EA, aVU, mF, hF, V, pl, q, o, hX, dP, x, lg, oz, ds, nX, dV, aYy, aT, h, dL, K, bxf, bc, aa, uQ, hM, A, aW, sp, vj, mQ, CM, ff, r, aGy, lo, CQ, en, iV, eE, aoG, f, bz, GR, cm, jg, ck, fT, eM, aDg, mx, at, eo, k, aEN, eg, y, bt, aJc); #c1(IL7R) c2(XP_005248356) c3(interleukin-7 receptor subunit alpha isoform X2) c4(268331, 268332, 268333, 268334) c5(268335, 268336, 268337, 268338, 268339) c6( ) c7(Kd, T, bM, dA, Ke, iW, jx, aA, aGx, cL, v, ci, EA, aVU, mF, hF, V, pl, q, o, hX, dP, x, lg, oz, ds, nX, dV, aYy, aT, h, dL, K, bxf, bc, aa, uQ, hM, A, aW, sp, vj, mQ, CM, ff, r, aGy, lo, CQ, en, iV, eE, aoG, f, bz, GR, cm, jg, ck, fT, eM, aDg, mx, at, eo, k, aEN, eg, y, bt, aJc); #c1(IL7R) c2(XP_005248357) c3(interleukin-7 receptor subunit alpha isoform X3) c4( ) c5(268340, 268341, 268342, 268343, 268344, 268345, 268346, 268347, 268348, 268349, 268350, 268351, 268352, 268353, 268354, 268355, 268356, 268357, 268358, 268359, 268360, 268361, 268362, 268363, 268364, 268365, 268366, 268367, 268368, 268369) c6( ) c7(Kd, T, bM, dA, Ke, iW, jx, aA, aGx, cL, v, ci, EA, aVU, mF, hF, V, pl, q, o, hX, dP, x, lg, oz, ds, nX, dV, aYy, aT, h, dL, K, bxf, bc, aa, uQ, hM, A, aW, sp, vj, mQ, CM, ff, r, aGy, lo, CQ, en, iV, eE, aoG, f, bz, GR, cm, jg, ck, fT, eM, aDg, mx, at, eo, k, aEN, eg, y, bt, aJc); #c1(IL9) c2(NP_000581) c3(interleukin-9 precursor) c4(13157) c5(13158, 13159, 13160, 13161, 13162, 13163) c6( ) c7(bM, sY, Dw, dA, fR, fG, aw, yG, ht, jh, fd, Hn, oY, j, ur, uu, bh, V, q, bRT, CJ, xT, dV, kH, cU, h, Ds, JK, ji, aDp, af, eu, aa, uQ, Nr, asp, A, je, sp, Y, fJ, afS, cr, r, dN, TA, eZ, po, hp, fe, jg, iV, eE, aKj, cM, boD, bz, uC, DF, vo, Nq, ck, kT, cT, Ta, ac, yR, Jr, iM, xO, Jp, uy, z, cj, Dt, eo, fD, adz, afT, y, Tc); #c1(IL9R) c2(NP_002177) c3(interleukin-9 receptor isoform 1 precursor) c4(126324) c5(126325, 126326, 126327, 126328, 126329, 126330) c6( ) c7(bM, sY, Dw, Ds, dL, ji, af, uQ, sp, Y, vj, hl, cr, eE, uC, bz, TA, cT, z, uy, Jp, y); #c1(IL9R) c2(NP_789743) c3(interleukin-9 receptor isoform 2) c4(252188, 252189) c5(252190, 252191, 252192, 252193, 252194, 252195, 252196, 252197, 252198, 252199) c6( ) c7(bM, sY, Dw, Ds, dl, ji, af, uQ, sp, Y, vj, hl, cr, eE, uC, bz, TA, cT, z, uy, Jp, y); #c1(IL9R) c2(XP_011543947) c3(interleukin-9 receptor isoform X1) c4( ) c5(343164, 343165) c6( ) c7(bM, sY, Dw, Ds, dL, ji, af, uQ, sp, Y, vj, hl, cr, eE, uC, bz, TA, cT, z, uy, Jp, y); #c1(IL9R) c2(XP_011543948) c3(interleukin-9 receptor isoform X2) c4( ) c5(343166) c6( ) c7(bM, sY, Dw, Ds, dL, ji, af, uQ, sp, Y, vj, hl, cr, eE, uC, bz, TA, cT, z, uy, Jp, y); #c1(IL9R) c2(XP_011543949) c3(interleukin-9 receptor isoform X3) c4(343167, 343168) c5( ) c6( ) c7(bM, sY, Dw, Ds, dl, ji, af, uQ, sp, Y, vj, hl, cr, eE, uC, bz, TA, cT, z, uy, Jp, y); #c1(IL9R) c2(XP_011543951) c3(interleukin-9 receptor isoform X4) c4( ) c5(343169, 343170, 343171, 343172, 343173) c6( ) c7(bM, sY, Dw, Ds, dl, ji, af, uQ, sp, Y, vj, hl, cr, eE, uC, bz, TA, cT, z, uy, Jp, y); #c1(IL9R) c2(XP_011543953) c3(interleukin-9 receptor isoform X6) c4( ) c5(343174, 343175, 343176, 343177, 343178, 343179) c6( ) c7(bM, sY, Dw, Ds, dL, ji, af, uQ, sp, Y, vj, hl, cr, eE, uC, bz, TA, cT, z, uy, Jp, y); #c1(ILDR1) c2(NP_001186728) c3(immunoglobulin-like domain-containing receptor 1 isoform 1 precursor) c4(78421) c5(78422, 78423, 78424, 78425, 78426, 78427) c6( ) c7(bRU, hH, V, bw); #c1(ILDR1) c2(NP_001186729) c3(immunoglobulin-like domain-containing receptor 1 isoform 3 precursor) c4(78428, 78429) c5(78430, 78431, 78432, 78433, 78434, 78435, 78436, 78437, 78438, 78439, 78440, 78441) c6( ) c7(bRU, hH, V, bw); #c1(ILDR1) c2(NP_787120) c3(immunoglobulin-like domain-containing receptor 1 isoform 2 precursor) c4(252128) c5(252129, 252130, 252131, 252132, 252133, 252134) c6( ) c7(bRU, hH, V, bw); #c1(ILDR1) c2(XP_005247446) c3(immunoglobulin-like domain-containing receptor 1 isoform X2) c4(267844) c5(267845, 267846, 267847, 267848, 267849, 267850) c6( ) c7(bRU, hH, V, bw); #c1(ILDR2) c2(NP_955383) c3(immunoglobulin-like domain-containing receptor 2 precursor) c4(261630) c5(261631, 261632, 261633, 261634, 261635, 261636) c6( ) c7(bA, cz); #c1(ILF2) c2(NP_001254738) c3(interleukin enhancer-binding factor 2 isoform 2) c4(94896) c5(94897, 94898, 94899, 94900, 94901) c6( ) c7(lg, if, AH, h, qN, Le); #c1(ILF2) c2(NP_004506) c3(interleukin enhancer-binding factor 2 isoform 1) c4(146992, 146993) c5(146994, 146995, 146996, 146997, 146998, 146999, 147000, 147001, 147002, 147003, 147004, 147005, 147006, 147007) c6( ) c7(lg, if, AH, h, qN, Le); #c1(ILF3) c2(NP_001131145) c3(interleukin enhancer-binding factor 3 isoform e) c4(52622) c5(52623, 52624, 52625, 52626, 52627, 52628) c6( ) c7(fR, ds, lh, AH, aw, oj, aT, uY, bl, eA, kU, cd, y); #c1(ILF3) c2(NP_004507) c3(interleukin enhancer-binding factor 3 isoform b) c4(147008) c5(147009, 147010, 147011, 147012, 147013, 147014, 147015, 147016, 147017, 147018) c6( ) c7(fR, ds, lh, AH, aw, oj, aT, uY, bl, eA, kU, cd, y); #c1(ILF3) c2(NP_036350) c3(interleukin enhancer-binding factor 3 isoform a) c4(173162, 173163) c5( ) c6( ) c7(fR, ds, lh, AH, aw, oj, aT, uY, bl, eA, KU, cd, y); #c1(ILF3) c2(NP_060090) c3(interleukin enhancer-binding factor 3 isoform d) c4(194092) c5(194093, 194094, 194095, 194096, 194097, 194098) c6( ) c7(fR, ds, lh, AH, aw, oj, aT, uY, bl, eA, KU, cd, y); #c1(ILF3) c2(NP_703194) c3(interleukin enhancer-binding factor 3 isoform c) c4(246097) c5(246098, 246099, 246100, 246101, 246102, 246103, 246104, 246105, 246106, 246107, 246108, 246109, 246110, 246111, 246112, 246113, 246114, 246115, 246116) c6( ) c7(fR, ds, lh, AH, aw, oj, aT, uY, bl, eA, kU, cd, y); #c1(ILK) c2(NP_001014795) c3(integrin-linked protein kinase isoform 1) c4(25983, 25984) c5(25985, 25986, 25987, 25988, 25989, 25990, 25991, 25992, 25993, 25994, 25995, 25996, 25997, 25998, 25999) c6( ) c7(bL, T, aC, dA, eR, ha, aA, eb, n, wV, Fb, j, ci, fr, fd, q, cd, ad, eA, eJ, fU, bv, hd, av, cc, fm, aT, cy, dL, ji, K, Tj, hB, Ha, ae, eL, d, Qr, HI, aW, Y, eH, r, fQ, cu, aLI, lo, vs, g, as, eF, LV, t, bz, aF, ck, bF, sS, eM, nB, dB, bE, X, mx, gZ, nL, Hs, dy, Hb, m, Xu, nz, bt, B); #c1(ILK) c2(NP_001265371) c3(integrin-linked protein kinase isoform 3) c4( ) c5(98378, 98379, 98380, 98381, 98382) c6( ) c7(bL, T, aC, dA, eR, ha, aA, eb, n, wV, Fb, j, ci, fr, fd, q, cd, ad, eA, eJ, fU, bv, hd, av, cc, fm, aT, cy, dL, ji, K, Tj, hB, Ha, ae, eL, d, Qr, HI, aW, Y, eH, r, fQ, cu, alJ, lo, vs, g, as, eF, LV, t, bz, aF, ck, bF, sS, eM, nB, dB, bE, X, mx, gZ, nl, Hs, dy, Hb, m, Xu, nz, bt, B); #c1(IMMP1L) c2(NP_001291203) c3(mitochondrial inner membrane protease subunit 1) c4(115548, 115549, 115550) c5(115551, 115552, 115553, 115554, 115555, 115556, 115557, 115558, 115559, 115560, 115561, 115562, 115563, 115564, 115565, 115566, 115567, 115568, 115569, 115570, 115571, 115572) c6( ) c7(bB, bE, T, yx, nJ); #c1(IMMP2L) c2(XP_005250687) c3(mitochondrial inner membrane protease subunit 2 isoform X1) c4(270475) c5(270476, 270477, 270478, 270479, 270480, 270481, 270482, 270483, 270484, 270485, 270486, 270487, 270488, 270489, 270490, 270491, 270492, 270493) c6( ) c7(bL, T, d, yL, au, EQ, he, cd, bN); #c1(IMMP2L) c2(XP_011514906) c3(mitochondrial inner membrane protease subunit 2 isoform X2) c4(307666) c5( ) c6( ) c7(bL, T, d, yL, au, EQ, he, cd, bN); #c1(IMMP2L) c2(XP_011514911) c3(mitochondrial inner membrane protease subunit 2 isoform X7) c4(307667) c5( ) c6( ) c7(bL, T, d, yL, au, EQ, he, cd, bN); #c1(IMMP2L) c2(XP_011514915) c3(mitochondrial inner membrane protease subunit 2 isoform X1I) c4( ) c5(307668, 307669, 307670, 307671, 307672, 307673, 307674, 307675, 307676, 307677, 307678, 307679) c6( ) c7(bL, T, d, yL, au, EQ, he, cd, bN); #c1(IMMT) c2(NP_001093639) c3(MICOS complex subunit MIC60 isoform 2) c4(40392) c5(40393, 40394, 40395, 40396, 40397, 40398) c6( ) c7(DS, T, eR, aj, bX, aY, V, aoz, J, ad, b, aE, aV, vc, K, ae, bjb, fe, cm, f, yf, Ew, dB, mG, i, iu); #c1(IMMT) c2(NP_001093640) c3(MICOS complex subunit MIC60 isoform 3) c4(40399, 40400) c5(40401, 40402, 40403, 40404, 40405, 40406, 40407, 40408, 40409, 40410, 40411, 40412) c6( ) c7(DS, T, eR, aj, bX, aY, V, aoz, J, ad, b, aE, aV, vc, K, ae, bjb, fe, cm, f, yf, Ew, dB, mG, i, iu); #c1(IMMT) c2(NP_006830) c3(MICOS complex subunit MIC60 isoform 1) c4(168504) c5(168505, 168506, 168507, 168508, 168509, 168510) c6( ) c7(DS, T, eR, aj, bX, aY, V, aoz, J, ad, b, aE, aV, vc, K, ae, bjb, fe, cm, f, yf, Ew, dB, mG, i, iu); #c1(IMMT) c2(XP_005264167) c3(MICOS complex subunit MIC60 isoform X1) c4(278678) c5(278679, 278680, 278681, 278682, 278683, 278684) c6( ) c7(DS, T, eR, aj, bX, aY, V, aoz, J, ad, b, aE, aV, vc, K, ae, bjb, fe, cm, f, yf, Ew, dB, mG, i, iu); #c1(IMMT) c2(XP_005264170) c3(MICOS complex subunit MIC60 isoform X2) c4(278685) c5(278686, 278687, 278688, 278689, 278690, 278691) c6( ) c7(DS, T, eR, aj, bX, aY, V, aoz, J, ad, b, aE, aV, vc, K, ae, bjb, fe, cm, f, yf, Ew, dB, mG, i, iu); #c1(IMMT) c2(XP_005264171) c3(MICOS complex subunit MIC60 isoform X3) c4(278692) c5(278693, 278694, 278695, 278696, 278697, 278698) c6( ) c7(DS, T, eR, aj, bX, aY, V, aoz, J, ad, b, aE, aV, vc, K, ae, bjb, fe, cm, f, yf, Ew, dB, mG, i, iu); #c1(IMP3) c2(NP_060755) c3(U3 small nucleolar ribonucleoprotein protein IMP3) c4(197609, 197610) c5(197611, 197612, 197613, 197614, 197615, 197616, 197617, 197618, 197619, 197620, 197621, 197622, 197623, 197624, 197625, 197626) c6( ) c7(Mb, T, hd, kP, aW, eH, aep, aT, dL, as, eF, eL, B); #c1(IMPA1) c2(NP_001138350) c3(inositol monophosphatase 1 isoform 2) c4(56248, 56249) c5(56250, 56251, 56252, 56253, 56254, 56255, 56256, 56257, 56258, 56259, 56260, 56261, 56262, 56263, 56264) c6( ) c7(bL, Si, T, ck, pF, bE, bB, auT, bk, ey, add); #c1(IMPA1) c2(NP_001138351) c3(inositol monophosphatase 1 isoform 3) c4(56265) c5(56266, 56267, 56268, 56269, 56270) c6( ) c7(bL, Si, T, ck, pF, bE, bB, auT, bK, ey, add); #c1(IMPA1) c2(NP_005527) c3(inositol monophosphatase 1 isoform 1) c4(157103, 157104) c5(157105, 157106, 157107, 157108, 157109, 157110, 157111, 157112, 157113, 157(14) c6( ) c7(bL, Si, T, ck, pF, bE, bB, auT, bK, ey, add); #c1(IMPA2) c2(NP_055029) c3(inositol monophosphatase 2) c4(178383, 178384) c5(178385, 178386, 178387, 178388, 178389, 178390, 178391, 178392, 178393, 178394, 178395) c6( ) c7(dP, ac, M, uJ, bS, bK, he, mY, eu); #c1(IMPA2) c2(XP_011523961) c3(inositol monophosphatase 2 isoform X1) c4( ) c5(318522) c6( ) c7(dP, ac, M, uJ, bS, bK, he, mY, eu); #c1(IMPACT) c2(NP_060909) c3(protein IMPACT) c4(198580) c5(198581, 198582, 198583, 198584, 198585, 198586) c6( ) c7(ck, mU, T, eM, kB, mW, e, mZ, mS, uJ, mY, j, ct, bS, bK, Z, mV, ae, mT, ad, mX); #c1(IMPAD1) c2(NP_060283) c3(inositol monophosphatase 3) c4(195195) c5(195196, 195197, 195198, 195199, 195200, 195201) c6( ) c7(ey, T, ln, bA, bRV); #c1(IMPDH1) c2(NP_000874) c3(inosine-5′-monophosphate dehydrogenase 1 isoform a) c4(16730) c5(16731, 16732, 16733, 16734, 16735, 16736) c6( ) c7(xX, lR, nb, W, bRX, bRY, aW, bRZ, aT, fe, nw, bRW, Nh, nC, ln, de); #c1(IMPDH1) c2(NP_001096075) c3(inosine-5′-monophosphate dehydrogenase 1 isoform c) c4(41050) c5(41051, 41052, 41053, 41054, 41055, 41056) c6( ) c7(xX, lR, nb, W, bRX, bRY, aW, bRZ, aT, fe, nw, bRW, Nh, nC, ln, de); #c1(IMPDH1) c2(NP_001136046) c3(inosine-5′-monophosphate dehydrogenase 1 isoform f) c4(54164) c5(54165, 54166, 54167, 54168, 54169, 54170) c6( ) c7(xX, lR, nb, W, bRX, bRY, aW, bRZ, aT, fe, nw, bRW, Nh, nC, ln, de); #c1(IMPDH1) c2(NP_001136047) c3(inosine-5′-monophosphate dehydrogenase 1 isoform g) c4(54171) c5(54172, 54173, 54174, 54175, 54176, 54177, 54178, 54179, 54180) c6( ) c7(xX, lR, nb, W, bRX, bRY, aW, bRZ, aT, fe, nw, bRW, Nh, nC, ln, de); #c1(IMPDH1) c2(NP_001136048) c3(inosine-5′-monophosphate dehydrogenase 1 isoform d) c4(54181) c5(54182, 54183, 54184, 54185, 54186, 54187) c6( ) c7(xX, lR, nb, W, bRX, bRY, aW, bRZ, aT, fe, nw, bRW, Nh, nC, ln, de); #c1(IMPDH1) c2(NP_001291450) c3(inosine-5′-monophosphate dehydrogenase 1 isoform h) c4(116067) c5(116068, 116069, 116070, 116071, 116072, 116073) c6( ) c7(xX, lR, nb, W, bRX, bRY, aW, bRZ, aT, fe, nw, bRW, Nh, nC, ln, de); #c1(IMPDH1) c2(NP_899066) c3(inosine-5′-monophosphate dehydrogenase 1 isoform b) c4(257945) c5(257946, 257947, 257948, 257949, 257950, 257951, 257952) c6( ) c7(xX, lR, nb, W, bRX, bRY, aW, bRZ, aT, fe, nw, bRW, Nh, nC, ln, de); #c1(IMPDH1) c2(XP_006716030) c3(inosine-5′-monophosphate dehydrogenase 1 isoform X1) c4( ) c5(290010) c6( ) c7(xX, IR, nb, W, bRX, bRY, aW, bRZ, aT, fe, nw, bRW, Nh, nC, ln, de); #c1(IMPDH2) c2(NP_000875) c3(inosine-5′-monophosphate dehydrogenase 2) c4(16737, 16738) c5(16739, 16740, 16741, 16742, 16743, 16744, 16745, 16746, 16747, 16748) c6( ) c7(gV, T, eR, hG, eH, aj, eF, y, ln); #c1(IMPDH2) c2(XP_006713191) c3(inosine-5′-monophosphate dehydrogenase 2 isoform X1) c4( ) c5(287056, 287057) c6( ) c7(gV, T, eR, hG, cH, aj, eF, y, ln); #c1(IMPG1) c2(NP_001269297) c3(interphotoreceptor matrix proteoglycan 1 isoform 2 precursor) c4(101055) c5(101056, 101057, 101058, 101059, 101060, 101061, 101062, 101063, 101064, 101065, 101066) c6( ) c7(IR, W, Sr, aw, k, db, bSa, Nh); #c1(IMPG1) c2(NP_001554) c3(interphotoreceptor matrix proteoglycan 1 isoform 1 precursor) c4(118918, 118919) c5(118920, 118921, 118922, 118923, 118924, 118925, 118926) c6( ) c7(IR, W, Sr, aw, k, db, bSa, Nh); #c1(IMPG2) c2(NP_057331) c3(interphotoreceptor matrix proteoglycan 2 precursor) c4(190163, 190164) c5(190165, 190166, 190167, 190168, 190169, 190170) c6( ) c7(aC, W, lR, U, nC, bSa, Nh, bSb); #c1(IMPG2) c2(XP_011511174) c3(interphotoreceptor matrix proteoglycan 2 isoform X2) c4( ) c5(301647, 301648, 301649, 301650, 301651, 301652) c6( ) c7(aC, W, lR, U, nC, bSa, Nh, bSb); #c1(INA) c2(NP_116116) c3(alpha-internexin) c4(225392) c5(225393, 225394, 225395, 225396, 225397, 225398) c6( ) c7(jf, dx, iF, d, dA, bb, WY, oV, RJ, U, fd, n, aaE, bSc, PJ, cm, Fc, aHd, eA, aR); #c1(INADL) c2(XP_011538767) c3(inaD-like protein isoform X3) c4(336495, 336496) c5(336497, 336498, 336499, 336500, 336501, 336502, 336503, 336504, 336505, 336506, 336507) c6( ) c7(iM, alx, je, aw, oc, lB, q, ad); #c1(INADL) c2(XP_011538769) c3(inaD-like protein isoform X6) c4( ) c5(336508) c6( ) c7(iM, alx, je, aw, oc, lB, q, ad); #c1(INCENP) c2(NP_001035784) c3(inner centromere protein isoform 1) c4(33057) c5(33058, 33059, 33060, 33061, 33062, 33063) c6( ) c7(Y, aF, aA); #c1(INCENP) c2(NP_064623) c3(inner centromere protein isoform 2) c4(203341, 203342) c5(203343, 203344, 203345, 203346, 203347, 203348, 203349, 203350, 203351, 203352, 203353, 203354, 203355, 203356, 203357, 203358, 203359, 203360) c6( ) c7(Y, aF, aA); #c1(INF2) c2(NP_001026884) c3(inverted formin-2 isoform 2) c4(29835) c5(29836) c6( ) c7(sk, vW, eR, bSe, bSd, vw, dg, dy, Bz, nA, bU, fu, aY); #c1(INF2) c2(NP_071934) c3(inverted formin-2 isoform 1) c4(212394, 212395) c5( ) c6( ) c7(sk, vW, eR, bSe, bSd, vw, dg, dy, Bz, nA, bU, fu, aY); #c1(INF2) c2(NP_116103) c3(inverted formin-2 isoform 3) c4( ) c5(225354, 225355, 225356, 225357, 225358, 225359) c6( ) c7(sk, vW, eR, bSe, bSd, vw, dg, dy, Bz, nA, bU, fu, aY); #c1(INF2) c2(XP_005268061) c3(inverted formin-2 isoform X1) c4( ) c5(281449, 281450, 281451, 281452, 281453, 281454) c6( ) c7(sk, vW, eR, bSe, bSd, vw, dg, dy, Bz, nA, bU, fu, aY); #c1(INF2) c2(XP_005268062) c3(inverted formin-2 isoform X2) c4( ) c5(281455, 281456, 281457, 281458, 281459, 281460) c6( ) c7(sk, vW, eR, bSe, bSd, vw, dg, dy, Bz, nA, bU, fu, aY); #c1(ING1) c2(NP_001254657) c3(inhibitor of growth protein 1 isoform E) c4( ) c5(94804) c6( ) c7(bM, T, aA, fd, ci, q, eA, afb, av, afQ, rC, aT, K, pk, qw, d, aW, Y, ff, eH, r, fe, aq, aF, en, g, as, t, eF, bz, cm, ik, iV, sS, Dy, eM, iT, dB, m, bt, ra, B); #c1(ING1) c2(NP_005528) c3(inhibitor of growth protein 1 isoform D) c4(157115) c5(157116, 157117, 157118, 157119, 157120, 157121) c6( ) c7(bM, T, aA, fd, ci, q, eA, afb, av, afQ, rC, aT, K, pk, qw, d, aW, Y, ff, eH, r, fe, aq, af, en, g, as, t, eF, bz, cm, ik, iV, sS, Dy, eM, iT, dB, m, bt, ra, B); #c1(ING1) c2(NP_937860) c3(inhibitor of growth protein 1 isoform C) c4(259335) c5(259336, 259337, 259338, 259339, 259340, 259341) c6( ) c7(bM, T, aA, fd, ci, q, eA, afb, av, afQ, rC, aT, K, pk, qw, d, aW, Y, ff, eH, r, fe, aq, af, en, g, as, t, eF, bz, cm, iK, iV, sS, Dy, eM, iT, dB, m, bt, ra, B); #c1(ING1) c2(NP_937861) c3(inhibitor of growth protein 1 isoform B) c4( ) c5(259342, 259343, 259344, 259345, 259346) c6( ) c7(bM, T, aA, fd, ci, q, eA, afb, av, afQ, rC, aT, K, pk, qw, d, aW, Y, ff, eH, r, fe, aq, aF, en, g, as, t, eF, bz, cm, iK, iV, sS, Dy, eM, iT, dB, m, bt, ra, B); #c1(ING1) c2(NP_937862) c3(inhibitor of growth protein 1 isoform A) c4(259347, 259348) c5(259349, 259350, 259351, 259352, 259353, 259354) c6( ) c7(bM, T, aA, fd, ci, q, eA, afb, av, afQ, rC, aT, K, pk, qw, d, aW, Y, ff, eH, r, fe, aq, af, en, g, as, t, eF, bz, cm, ik, iV, sS, Dy, eM, iT, dB, m, bt, ra, B); #c1(ING2) c2(NP_001278888) c3(inhibitor of growth protein 2 isoform 2) c4(110812) c5(110813, 110814, 110815, 110816, 110817, 110818) c6( ) c7(T, aMv, aA, jh, Ul, q, ip, av, qp, aT, h, dL, K, eL, aW, Y, ff, r, aF, en, g, cm, ik, eM, B); #c1(ING2) c2(NP_001555) c3(inhibitor of growth protein 2 isoform 1) c4(118927) c5(118928, 118929, 118930, 118931, 118932, 118933) c6( ) c7(T, aMv, aA, jh, Ul, q, ip, av, qp, aT, h, dL, K, eL, aW, Y, ff, r, af, en, g, cm, ik, eM, B); #c1(ING3) c2(NP_061944) c3(inhibitor of growth protein 3 isoform 1) c4(201243, 201244) c5(201245, 201246, 201247, 201248, 201249, 201250) c6( ) c7(GI, T, aMv, aA, hy, Ul, q, aT, K, cp, aW, Y, ff, r, aF, en, g, iK, bN, Dy, eM, B); #c1(ING3) c2(NP_938008) c3(inhibitor of growth protein 3 isoform 3) c4( ) c5(259499, 259500, 259501, 259502, 259503, 259504) c6( ) c7(GI, T, aMv, aA, hy, Ul, q, aT, K, cp, aW, Y, ff, r, aF, en, g, ik, bN, Dy, eM, B); #c1(ING4) c2(NP_001121054) c3(inhibitor of growth protein 4 isoform 9) c4(45085) c5(45086, 45087, 45088, 45089, 45090, 45091) c6( ) c7(T, fG, fd, bB, hy, q, ad, eA, iu, aE, aT, K, ae, d, JI, aW, Y, aF, g, cl, as, t, ik, bz, LB, eM, dB, bE, AU, m, B); #c1(ING4) c2(NP_001121055) c3(inhibitor of growth protein 4 isoform 3) c4(45092) c5(45093, 45094, 45095, 45096, 45097, 45098) c6( ) c7(T, fG, fd, bB, hy, q, ad, eA, iu, aE, aT, K, ae, d, Jl, aW, Y, aF, g, cl, as, t, ik, bz, LB, eM, dB, bE, AU, m, B); #c1(ING4) c2(NP_001121056) c3(inhibitor of growth protein 4 isoform 4) c4(45099) c5(45100, 45101, 45102, 45103, 45104, 45105) c6( ) c7(T, fG, fd, bB, hy, q, ad, eA, iu, aE, aT, K, ae, d, JI, aW, Y, aF, g, cl, as, t, ik, bz, LB, eM, dB, bE, AU, m, B); #c1(ING4) c2(NP_001121057) c3(inhibitor of growth protein 4 isoform 5) c4(45106) c5(45107, 45108, 45109, 45110, 45111, 45112) c6( ) c7(T, fG, fd, bB, hy, q, ad, eA, iu, aE, aT, K, ae, d, JI, aW, Y, aF, g, cl, as, t, iK, bz, LB, eM, dB, bE, AU, m, B); #c1(ING4) c2(NP_001121058) c3(inhibitor of growth protein 4 isoform 6) c4(45113) c5(45114, 45115, 45116, 45117, 45118, 45119) c6( ) c7(T, fG, fd, bB, hy, q, ad, eA, iu, aE, aT, K, ae, d, JI, aW, Y, aF, g, cl, as, t, ik, bz, LB, eM, dB, bE, AU, m, B); #c1(ING4) c2(NP_057246) c3(inhibitor of growth protein 4 isoform 1) c4(189708) c5(189709, 189710, 189711, 189712, 189713, 189714) c6( ) c7(T, fG, fd, bB, hy, q, ad, eA, iu, aE, aT, K, ae, d, JI, aW, Y, aF, g, cl, as, t, ik, bz, LB, eM, dB, bE, AU, m, B); #c1(ING5) c2(NP_115705) c3(inhibitor of growth protein 5) c4(223654) c5(223655, 223656, 223657, 223658, 223659, 223660, 223661) c6( ) c7(bt, aE, ci, aF, aZF); #c1(ING5) c2(XP_011510291) c3(inhibitor of growth protein 5 isoform X3) c4(300871) c5(300872, 300873, 300874, 300875, 300876) c6( ) c7(bt, aE, ci, aF, aZF); #c1(INHA) c2(NP_002182) c3(inhibin alpha chain preproprotein) c4(126361) c5(126362, 126363, 126364, 126365, 126366, (26367) c6( ) c7(bM, T, bcb, apr, aA, cz, eb, Sv, ci, qN, fr, iJ, ad, UE, dP, il, BJ, bDJ, aT, iv, h, ae, kl, bbs, aF, Hu, g, bu, cl, zZ, f, t, uC, sn, AM, Lb, m, MC, B); #c1(INHBA) c2(NP_002183) c3(inhibin beta A chain precursor) c4(126368) c5(126369, 126370, 126371, 126372, 126373, 126374, 126375, 126376, 126377, 126378) c6( ) c7(ka, os, T, kh, pf, aw, aA, eb, oZ, v, fr, ci, lu, cd, ad, dP, iu, hw, pN, aT, Dz, kl, ff, r, aF, en, bu, g, t, uC, op, c, ki, hL, Lb, m, UF, y, B); #c1(INHBB) c2(NP_002184) c3(inhibin beta B chain preproprotein) c4(126379) c5(126380, 126381, 126382, 126383, 126384, 126385, 126386) c6( ) c7(UH, kl, s, arA, il, iX, r, aT, cz, v, Lb, m, ci, bu, Ul, cd, ad, UE); #c1(INHBC) c2(NP_005529) c3(inhibin beta C chain preproprotein) c4(157122) c5(157123, 157124, 157125, 157126, 157127, 157128) c6( ) c7(W, eM, s, Lb, m, bu); #c1(INHBE) c2(NP_113667) c3(inhibin beta E chain precursor) c4(222026) c5(222027, 222028, 222029, 222030, 222031, 222032) c6( ) c7(ID, T, kh, aZ, aw, bB, ci, BY, dP, hd, Cj, aE, kl, BJ, kf, bl, K, cp, ly, yo, s, Y, fQ, kp, Hu, bu, t, lx, bP, nB, w, Lb, m, pw, du); #c1(INO80) c2(NP_060023) c3(DNA helicase INO80) c4(193732, 193733) c5(193734, 193735, 193736, 193737, 193738, 193739, 193740, 193741, 193742, 193743) c6( ) c7(Kw); #c1(INO80) c2(XP_011519988) c3(DNA helicase INO80 isoform X2) c4( ) c5(314849, 314850, 314851, 314852, 314853, 314854) c6( ) c7(Kw); #c1(INO80B) c2(NP_112578) c3(INO80 complex subunit B) c4(221464, 221465) c5(221466, 221467, 221468, 221469, 221470, 221471, 221472, 221473, 221474, 221475, 221476, 221477) c6( ) c7(ae, ad); #c1(INO80D) c2(NP_060229) c3(INO80 complex subunit D) c4(194869, 194870, 194871) c5(194872, 194873, 194874, 194875, 194876, 194877, 194878, 194879, 194880, 194881, 194882, 194883, 194884, 194885, 194886, 194887, 194888, 194889) c6( ) c7(EQ); #c1(INPP1) c2(NP_001122400) c3(inositol polyphosphate 1-phosphatase) c4(46686, 46687, 46688, 46689, 46690, 46691, 46692, 46693, 46694) c5(46695, 46696, 46697, 46698, 46699, 46700, 46701, 46702, 46703, 46704, 46705, 46706, 46707, 46708, 46709, 46710, 46711, 46712, 46713, 46714, 46715, 46716, 46717, 46718, 46719, 46720, 46721, 46722, 46723, 46724, 46725, 46726, 46727, 46728, 46729, 46730, 46731, 46732, 46733, 46734, 46735, 46736, 46737, 46738, 46739, 46740, 46741, 46742, 46743, 46744, 46745, 46746, 46747, 46748, 46749, 46750, 46751, 46752, 46753, 46754, 46755, 46756) c6( ) c7(bL, T, bP, Ei, r, bK, mY, q, bN); #c1(INPP4A) c2(NP_001127696) c3(type 1 inositol 3,4-bisphosphate 4-phosphatase isoform d) c4(49238) c5(49239, 49240, 49241, 49242, 49243, 49244, 49245, 49246, 49247, 49248, 49249, 49250) c6( ) c7(z, bM, nA); #c1(INPP4A) c2(NP_001127697) c3(type 1 inositol 3,4-bisphosphate 4-phosphatase isoform c) c4(49251) c5(49252, 49253, 49254, 49255, 49256, 49257) c6( ) c7(z, bM, nA); #c1(INPP4A) c2(NP_001557) c3(type 1 inositol 3,4-bisphosphate 4-phosphatase isoform b) c4(118941) c5(118942, 118943, 118944, 118945, 118946, 118947) c6( ) c7(z, bM, nA); #c1(INPP4A) c2(NP_004018) c3(type 1 inositol 3,4-bisphosphate 4-phosphatase isoform a) c4(142880, 142881) c5(142882, 142883, 142884, 142885, 142886, 142887, 142888, 142889, 142890, 142891, 142892, 142893) c6( ) c7(z, bM, nA); #c1(INPP4B) c2(NP_001095139) c3(type 11 inositol 3,4-bisphosphate 4-phosphatase) c4(40815) c5(40816, 40817, 40818, 40819, 40820, 40821, 40822, 40823, 40824, 40825, 40826, 40827, 40828, 40829, 40830, 40831, 40832, 40833, 40834, 40835, 40836, 40837, 40838, 40839, 40840, 40841, 40842, 40843, 40844, 40845, 40846) c6( ) c7(ck, T, kP, aW, Y, aT, n, Gn, m, t, rA, ae, ad, bA); #c1(INPP4B) c2(XP_005263380) c3(type 11 inositol 3,4-bisphosphate 4-phosphatase isoform X7) c4(278462) c5(278463, 278464, 278465, 278466, 278467) c6( ) c7(ck, T, kP, aW, Y, aT, n, Gn, m, t, rA, ae, ad, bA); #c1(INPP4B) c2(XP_006714467) c3(type 11 inositol 3,4-bisphosphate 4-phosphatase isoform X1l) c4( ) c5(287845, 287846, 287847, 287848, 287849, 287850, 287851, 287852, 287853, 287854, 287855, 287856, 287857, 287858, 287859, 287860, 287861, 287862, 287863) c6( ) c7(ck, T, kP, aW, Y, aT, n, Gn, m, t, rA, ae, ad, bA); #c1(INPP4B) c2(XP_011530693) c3(type 11 inositol 3,4-bisphosphate 4-phosphatase isoform X2) c4( ) c5(326756, 326757, 326758, 326759, 326760) c6( ) c7(ck, T, kP, aW, Y, aT, n, Gn, m, t, rA, ae, ad, bA); #c1(INPP4B) c2(XP_011530694) c3(type 11 inositol 3,4-bisphosphate 4-phosphatase isoform X3) c4(326761, 326762, 326763, 326764) c5(326765, 326766, 326767, 326768, 326769, 326770) c6( ) c7(ck, T, kP, aW, Y, aT, n, Gn, m, t, rA, ae, ad, bA); #c1(INPP4B) c2(XP_011530695) c3(type 11 inositol 3,4-bisphosphate 4-phosphatase isoform X5) c4(326771, 326772) c5( ) c6( ) c7(ck, T, kP, aW, Y, aT, n, Gn, m, t, rA, ae, ad, bA); #c1(INPP4B) c2(XP_011530698) c3(type 11 inositol 3,4-bisphosphate 4-phosphatase isoform X10) c4(326773) c5( ) c6( ) c7(ck, T, kP, aW, Y, aT, n, Gn, m, t, rA, ae, ad, bA); #c1(INPP5A) c2(NP_005530) c3(type 1 inositol 1,4,5-trisphosphate 5-phosphatase) c4(157129, 157130, 157131, 157132, 157133, 157134, 157135, 157136, 157137, 157138) c5(157139, 157140, 157141, 157142, 157143, 157144, 157145, 157146, 157147, 157148, 157149, 157150, 157151, 157152, 157153, 157154, 157155, 157156, 157157, 157158, 157159, 157160, 157161, 157162, 157163, 157164, 157165, 157166, 157167, 157168, 157169, 157170, 157171, 157172, 157173, 157174, 157175, 157176, 157177, 157178, 157179, 157180) c6( ) c7(adg, qy, KW, ks); #c1(INPP5A) c2(XP_011538070) c3(type 1 inositol 1,4,5-trisphosphate 5-phosphatase isoform X1) c4( ) c5(335616, 335617, 335618, 335619, 335620, 335621, 335622, 335623, 335624, 335625, 335626, 335627, 335628, 335629, 335630, 335631, 335632, 335633) c6( ) c7(adg, qy, KW, ks); #c1(INPP5B) c2(NP_001284363) c3(type 11 inositol 1,4,5-trisphosphate 5-phosphatase isoform 2 precursor) c4(111805, 111806, 111807, 111808) c5(111809, 111810, 111811, 111812, 111813, 111814, 111815, 111816, 111817, 111818, 111819, 111820, 111821, 111822, 111823, 111824, 111825, 111826, 111827, 111828, 111829, 111830) c6( ) c7(abV, KW); #c1(INPP5B) c2(XP_011539690) c3(type 11 inositol 1,4,5-trisphosphate 5-phosphatase isoform X2) c4( ) c5(337924, 337925, 337926) c6( ) c7(abV, KW); #c1(INPP5B) c2(XP_011539693) c3(type 11 inositol 1,4,5-trisphosphate 5-phosphatase isoform X6) c4( ) c5(337927, 337928, 337929, 337930, 337931, 337932) c6( ) c7(abV, KW); #c1(INPP5B) c2(XP_011539695) c3(type 11 inositol 1,4,5-trisphosphate 5-phosphatase isoform X7) c4( ) c5(337933, 337934, 337935, 337936, 337937, 337938) c6( ) c7(abV, KW); #c1(INPP5D) c2(NP_001017915) c3(phosphatidylinositol 3,4,5-trisphosphate 5-phosphatase 1 isoform a) c4(26613, 26614, 26615, 26616, 26617) c5(26618, 26619, 26620, 26621, 26622, 26623, 26624, 26625, 26626, 26627, 26628, 26629) c6( ) c7(ck, pq, T, dD, F, fM, iT, iU, ea, jx, fd, bw, DO, lo, dz, v, eq, bi, hF, bt); #c1(INPP5D) c2(NP_005532) c3(phosphatidylinositol 3,4,5-trisphosphate 5-phosphatase 1 isoform b) c4(157181) c5(157182, 157183, 157184, 157185, 157186, 157187, 157188, 157189, 157190, 157191, 157192, 157193, 157194, 157195, 157196, 157197, 157198, 157199, 157200, 157201, 157202, 157203, 157204, 157205, 157206, 157207, 157208, 157209, 157210, 157211, 157212) c6( ) c7(ck, pq, T, dD, F, fM, iT, iU, ea, jx, fd, bw, DO, lo, dz, v, eq, bi, hF, bt); #c1(INPP5D) c2(XP_011547153) c3(phosphatidylinositol 3,4,5-trisphosphate 5-phosphatase 1 isoform X1) c4(345876) c5(345877, 345878, 345879, 345880, 345881, 345882) c6( ) c7(ck, pq, T, dD, F, fM, iT, iU, ea, jx, fd, bw, DO, lo, dz, v, eq, bi, hF, bt); #c1(INPP5E) c2(NP_063945) c3(72 kDa inositol polyphosphate 5-phosphatase) c4(202387) c5(202388, 202389, 202390, 202391, 202392, 202393) c6( ) c7(aPt, Pf, Y, KW, aSW, v, bSf, nA, Tp); #c1(INPP5F) c2(NP_001230123) c3(phosphatidylinositide phosphatase SAC2 isoform 2) c4(85952) c5(85953, 85954, 85955, 85956, 85957, 85958) c6( ) c7(oe); #c1(INPP5F) c2(NP_001230124) c3(phosphatidylinositide phosphatase SAC2 isoform 3) c4(85959) c5(85960, 85961, 85962, 85963, 85964, 85965) c6( ) c7(De); #c1(INPP5F) c2(NP_055752) c3(phosphatidylinositide phosphatase SAC2 isoform 1) c4(183284, 183285) c5(183286, 183287, 183288, 183289, 183290, 183291) c6( ) c7(ce); #c1(INPP5F) c2(XP_006717783) c3(phosphatidylinositide phosphatase SAC2 isoform X4) c4( ) c5(291356, 291357, 291358, 291359, 291360, 291361) c6( ) c7(oe); #c1(INPP5K) c2(NP_057616) c3(inositol polyphosphate 5-phosphatase K isoform 1) c4(191790, 191791, 191792, 191793) c5(191794, 191795, 191796, 191797, 191798, 191799, 191800, 191801, 191802, 191803) c6( ) c7(Dy, Y, eW, Ex, kk); #c1(INPP5K) c2(XP_005256740) c3(inositol polyphosphate 5-phosphatase K isoform X1) c4( ) c5(274306, 274307, 274308, 274309, 274310, 274311, 274312, 274313, 274314, 274315, 274316, 274317, 274318, 274319, 274320, 274321, 274322) c6( ) c7(Dy, Y, eW, Ex, kk); #c1(INPP5K) c2(XP_005256743) c3(inositol polyphosphate 5-phosphatase K isoform X2) c4( ) c5(274323, 274324, 274325, 274326, 274327) c6( ) c7(Dy, Y, eW, Ex, kk); #c1(INPP5K) c2(XP_011522238) c3(inositol polyphosphate 5-phosphatase K isoform X3) c4(316870) c5(316871, 316872, 316873, 316874, 316875, 316876) c6( ) c7(Dy, Y, eW, Ex, kk); #c1(INPPL1) c2(NP_001558) c3(phosphatidylinositol 3,4,5-trisphosphate 5-phosphatase 2) c4(118948, 118949, 118950) c5(118951, 118952, 118953, 118954, 118955, 118956, 118957, 118958, 118959, 118960, 118961, 118962, 118963, 118964, 118965) c6( ) c7(bL, T, cz, bB, fd, aj, v, ci, J, bsH, aT, A, d, B, iU, aW, qa, bSg, bE, bo, ei, X); #c1(INPPL1) c2(XP_005274035) c3(phosphatidylinositol 3,4,5-trisphosphate 5-phosphatase 2 isoform X2) c4(284905) c5(284906, 284907, 284908, 284909, 284910) c6( ) c7(bL, T, cz, bB, fd, aj, v, ci, J, bsH, aT, A, d, B, iU, aW, qa, bSg, bE, bo, ei, X); #c1(INPPL1) c2(XP_011543302) c3(phosphatidylinositol 3,4,5-trisphosphate 5-phosphatase 2 isoform X3) c4(342087, 342088) c5(342089, 342090, 342091, 342092, 342093, 342094, 342095) c6( ) c7(bL, T, cz, bB, fd, aj, v, ci, J, bsH, aT, A, d, B, iU, aW, qa, bSg, bE, bo, ei, X); #c1(INS) c2(NP_001278826) c3(insulin preproprotein) c4(110156, 110157, 110158, 110159, 110160, 110161, 110162) c5(110163, 110164, 110165, 110166, 110167, 110168, 110169, 110170, 110171, 110172, 110173, 110174, 110175, 110176, 110177, 110178, 110179, 110180, 110181, 110182, 110183, 110184, 110185, 110186, 110187, 110188, 110189, 110190, 110191, 110192, 110193, 110194, 110195, 110196, 110197, 110198, 110199, 110200, 110201, 110202, 110203, 110204, 110205, 110206, 110207, 110208, 110209, 110210, 110211, 110212, 110213, 110214, 110215, 110216, 110217, 110218, 110219, 110220, 110221, 110222, 110223, 110224, 110225, 110226, 110227, 110228, 110229, 110230, 110231, 110232, 110233, 110234, 110235, 110236, 110237, 110238, 110239, 110240, 110241, 110242, 110243, 110244, 110245, 110246, 110247, 110248, 110249, 110250, 110251, 110252, 110253, 110254, 110255, 110256, 110257, 110258, 110259, 110260, 110261, 110262, 110263, 110264, 110265, 110266, 110267, 110268, 110269, 110270, 110271, 110272, 110273, 110274, 110275, 110276, 110277, 110278, 110279, 110280, 110281, 110282, 110283, 110284, 110285, 110286, 110287, 110288, 110289, 110290, 110291, 110292, 110293, 110294, 110295, 110296, 110297, 110298, 110299, 110300, 110301, 110302, 110303, 110304, 110305, 110306, 110307, 110308, 110309, 110310, 110311, 110312, 110313, 110314, 110315, 110316, 110317, 110318, 110319, 110320, 110321, 110322, 110323, 110324, 110325, 110326, 110327, 110328, 110329, 110330, 110331, 110332, 110333, 110334, 110335, 110336, 110337, 110338, 110339, 110340, 110341, 110342, 110343, 110344, 110345, 110346, 110347, 110348, 110349, 110350, 110351, 110352, 110353, 110354, 110355, 110356, 110357, 110358, 110359, 110360, 110361, 110362, 110363, 110364, 110365, 110366, 110367, 110368, 110369, 110370, 110371, 110372, 110373, 110374, 110375, 110376, 110377, 110378, 110379, 110380, 110381, 110382, 110383, 110384, 110385, 110386, 110387, 110388, 110389, 110390, 110391, 110392, 110393, 110394, 110395, 110396, 110397, 110398, 110399, 110400, 110401, 110402, 110403, 110404, 110405, 110406, 110407, 110408, 110409, 110410, 110411, 110412, 110413, 110414, 110415, 110416, 110417, 110418, 110419, 110420, 110421, 110422, 110423, 110424, 110425, 110426, 110427, 110428, 110429, 110430, 110431, 110432, 110433, 110434, 110435, 110436, 110437, 110438, 110439, 110440, 110441, 110442, 110443, 110444, 110445, 110446, 110447, 110448, 110449, 110450, 110451, 110452, 110453, 110454, 110455, 110456, 110457, 110458, 110459, 110460, 110461, 110462, 110463, 110464, 110465, 110466, 110467, 110468, 110469, 110470, 110471, 110472, 110473, 110474, 110475, 110476, 110477, 110478, 110479, 110480, 110481, 110482, 110483, 110484, 110485, 110486, 110487, 110488, 110489, 110490, 110491, 110492, 110493, 110494, 110495, 110496, 110497, 110498, 110499, 110500, 110501, 110502, 110503, 110504, 110505, 110506, 110507, 110508, 110509, 110510, 110511, 110512, 110513, 110514, 110515, 110516, 110517, 110518, 110519, 110520, 110521, 110522, 110523, 110524, 110525, 110526, 110527, 110528, 110529, 110530, 110531, 110532, 110533, 110534, 110535, 110536, 110537, 110538, 110539, 110540, 110541, 110542, 110543, 110544, 110545, 110546, 110547, 110548, 110549, 110550, 110551, 110552, 110553, 110554, 110555, 110556, 110557, 110558, 110559, 110560, 110561, 110562, 110563, 110564, 110565, 110566, 110567, 110568, 110569, 110570, 110571, 110572, 110573, 110574, 110575, 110576, 110577, 110578, 110579, 110580, 110581, 110582, 110583, 110584, 110585, 110586, 110587, 110588, 110589, 110590, 110591, 110592, 110593, 110594, 110595, 110596, 110597, 110598, 110599, 110600, 110601, 110602, 110603, 110604, 110605, 110606, 110607, 110608, 110609, 110610, 110611, 110612, 110613, 110614, 110615, 110616, 110617, 110618, 110619, 110620, 110621, 110622, 110623, 110624, 110625, 110626, 110627, 110628, 110629, 10630, 110631, 110632, 110633, 110634, 10635, 110636, 110637, 110638, 110639, 110640, 110641, 110642, 110643, 110644, 110645, 110646, 110647, 110648, 110649, 110650, 110651, 110652, 110653, 110654, 110655) c6(110656, 110657, 110658, 110659, 110660, 110661, 110662, 110663, 110664, 110665, 110666, 110667, 110668, 110669, 110670, 110671, 110672, 110673, 110674, 110675, 110676) c7(bSR, T, os, aoq, ea, abM, bO, fd, ci, aY, GK, bv, aex, aE, bJj, bSC, dV, BZ, iv, hu, li, ly, yo, Ls, Fe, nY, uY, YG, dg, bSE, GR, iA, nB, dc, wE, cj, azg, k, de, ZP, FG, ht, cQ, akA, aj, ww, ml, cd, br, td, aow, aZv, rC, adX, KN, ey, bl, on, yi, Ln, MD, ahJ, pm, zZ, QK, iO, RB, ky, dB, Fi, eD, JO, bSP, TF, E, mT, bSO, cz, aA, GP, avO, bOI, Tq, nS, q, Wu, Ql, yu, cy, rO, bSz, rz, dl, bA, wr, bSK, aeS, lo, uU, cl, pb, tR, pa, rR, iQ, ac, pO, tW, awN, mG, aDG, hr, cG, bSD, bt, rA, ars, ao, aag, iW, bAg, jN, jh, bgj, oZ, dG, bST, fu, bSj, ad, hX, abZ, LL, vJ, Dq, QJ, rx, bj, lQ, bSo, xX, lx, CH, PE, cW, Mz, dq, iV, bDu, bSJ, bz, mi, ag, lZ, lC, iu, lD, Zz, if, e, bB, cL, bSh, aTS, brq, ip, x, bSi, ws, Pq, Gb, bSN, bc, azu, Hd, aCg, JI, fc, hl, as, eF, cm, bw, aiv, mZ, aac, bSA, Kv, vA, vy, ym, Vj, dD, gU, Hm, yG, bSQ, n, nA, aje, aCi, RD, md, jz, aLs, ec, Do, Bj, gd, bzd, aAG, mV, aeJ, mU, avP, A, bAF, Rj, lb, atf, yq, afj, dC, hL, tl, GN, B, dA, mr, aoH, ti, aNG, v, GS, V, qH, zT, aPW, DK, wN, rS, bza, aql, MI, rD, bSH, aKG, ae, jf, hM, ev, asy, aGp, bSI, bu, t, nC, ck, bSM, bAn, cl, kP, wO, lN, au, bSv, gX, Gg, zu, yv, vi, nZ, hl, pY, ml, vG, wV, eq, bSk, aoz, hd, ds, hD, vc, Dj, sj, d, aVd, Z, adj, dr, gk, akQ, pP, ll, atg, gZ, Km, y, aXu, aC, bzx, al, gf, bSt, xE, u, pX, M, K, bLj, dF, eL, ago, bbC, lW, aNb, adE, aB, g, uC, iP, wT, bAh, ou, avS, AM, U, Hb, m, Uw, abX, lV, zX, bSp, DI, NT, aw, fg, JW, bKr, ft, mh, nV, lt, bSG, uV, ZN, dx, RE, bSu, aev, bSS, jT, aHr, JP, ar, kT, GG, W, anZ, bo, yP, mg, ZG, it, ZV, wj, fG, oe, jx, ql, wo, mS, hz, j, sl, J, o, er, jF, MR, bBN, qG, bq, hK, Fg, kl, Y, lW, LO, r, bSy, wA, Qi, bmS, c, kB, hc, aNJ, dh, bSs, JL, cS, bSr, dy, UF, gT, bSL, gx, kh, aZ, LX, bs, bS, pl, EA, hF, dP, fU, sN, aet, UG, oj, vB, tt, bKm, aOh, vl, uX, lR, xs, eH, cr, bSn, hp, iR, Bu, agn, qa, bJt, dR, pu, Lj, bib, cP, bC, ei, sT, hE, xg, bSw, st, Fj, pf, eb, amj, aoM, aR, dY, ata, il, iX, qM, de, H, bhm, pk, hm, tw, aW, aeC, hJ, avx, eM, wB, nw, aYS, R, aFc, ko, bKx, fr, ct, qN, vY, rc, eW, biN, bSq, BI, cc, Mm, bAi, PD, h, jy, dL, aKE, hB, mY, vx, Dg, OV, asp, aq, kp, Hu, kk, mb, op, lx, mX, bJ, arF, JR, adZ, Uv, hR, bSB, bL, eR, sM, CR, tc, asA, C, so, Ns, Zp, mj, GL, uT, qp, aT, bSx, bK, an, af, ak, bSI, s, iU, oV, bAv, bSm, OS, hf, OC, BU, sS, nM, aGo, fP, mf, aoJ, Kd, bSF, ha, vr, jE, tK, qC, nH, l, F, ot, xl, oz, mW, sO, ts, aNR, fb, iL, yF, qt, eZ, yy, gl, aF, ek, bOM, LB, es, mO, bE, z, arC, awk, aS, pk); #c1(INSC) c2(NP_001027024) c3(protein inscuteable homolog isoform a) c4(30105) c5(30106, 30107, 30108, 30109, 30110, 30111, 30112, 30113, 30114, 30115) c6( ) c7(ac, ad); #c1(INSC) c2(NP_001265242) c3(protein inscuteable homolog isoform b) c4(98039) c5(98040, 98041, 98042, 98043, 98044, 98045, 98046, 98047) c6( ) c7(ac, ad); #c1(INSC) c2(NP_001265243) c3(protein inscuteable homolog isoform c) c4(98048) c5(98049, 98050, 98051, 98052, 98053, 98054) c6( ) c7(ac, ad); #c1(INSC) c2(NP_001265245) c3(protein inscuteable homolog isoform e) c4(98055) c5(98056, 98057, 98058, 98059, 98060, 98061, 98062, 98063) c6( ) c7(ac, ad); #c1(INS1G1) c2(NP_005533) c3(insulin-induced gene 1 protein isoform 1) c4(157213) c5(157214, 157215, 157216, 157217, 157218, 157219) c6( ) c7(bL, Zo, T, dD, ea, aw, aA, v, j, ci, eq, zP, ad, K, dQ, af, zQ, fc, aq, aF, gZ, cP, ei, B); #c1(INS1G1) c2(NP_938150) c3(insulin-induced gene 1 protein isoform 2) c4(259640) c5(259641, 259642, 259643, 259644, 259645, 259646) c6( ) c7(bL, Zo, T, dD, ea, aw, aA, v, j, ci, eq, zP, ad, K, dQ, af, zQ, fc, aq, aF, gZ, cP, ei, B); #c1(INS1G1) c2(NP_938151) c3(insulin-induced gene 1 protein isoform 3) c4(259647) c5(259648, 259649, 259650, 259651, 259652, 259653, 259654, 259655, 259656, 259657, 259658, 259659) c6( ) c7(bL, Zo, T, dD, ea, aw, aA, v, j, ci, eq, zP, ad, K, dQ, af, zQ, fc, aq, aF, gZ, cP, ei, B); #c1(INS1G1) c2(XP_005249600) c3(insulin-induced gene 1 protein isoform X1) c4(269712) c5( ) c6( ) c7(bL, Zo, T, dD, ea, aw, aA, v, j, ci, eq, zP, ad, K, dQ, af, zQ, fc, aq, aF, gZ, cP, ei, B); #c1(INS1G2) c2(XP_011509594) c3(insulin-induced gene 2 protein isoform X1) c4(300168) c5(300169, 300170, 300171, 300172, 300173, 300174) c6( ) c7(bM, dD, aw, bB, aj, v, j, eq, V, cd, J, aE, arA, aT, qM, cH, aW, qt, mo, LV, bJ, bE, gZ, ei, X); #c1(INS-IGF2) c2(NP_001035835) c3(insulin, isoform 2 precursor) c4(33204) c5(33205, 33206, 33207, 33208, 33209, 33210, 33211, 33212, 33213) c6(33214, 33215) c7(bSR, T, os, aoq, ea, abM, fd, ci, aY, GK, bv, aex, aE, bJj, dV, iv, BZ, hu, li, ly, yo, Ls, Fe, nY, uY, YG, dg, bSE, GR, iA, nB, dc, wE, cj, azg, k, ZP, FG, ht, cQ, akA, aj, ml, cd, br, aZv, rC, adX, KN, ey, bl, on, yi, Ln, MD, ahJ, pm, zZ, QK, iO, ky, dB, Fi, eD, JQ, bSP, TF, mT, bSO, cz, aA, avO, bol, Tq, nS, q, Wu, Ql, yu, cy, rO, bSz, bA, wr, bSK, lo, uU, cl, pb, tR, pa, rR, iQ, ac, pO, tW, awN, mG, aDG, hr, cG, bSD, bt, rA, ars, ao, aag, iW, bAg, jN, jh, bgj, oZ, dG, bST, fu, bSj, ad, hX, LL, vJ, Dq, QJ, rX, bj, lQ, bSo, lx, cH, PE, cW, Mz, dq, iV, bDu, bSJ, bz, mi, ag, lZ, lC, iu, lD, Zz, if, e, bB, bSh, aTS, brq, ip, x, ws, Pq, Gb, bc, azu, Hd, aCg, JI, fc, hl, as, eF, cm, bw, aiv, mZ, aac, Kv, vA, vy, ym, Vj, dD, gU, Hm, yG, n, nA, aje, RD, jz, aLs, ec, Do, Bj, gd, bzd, aAG, mV, aeJ, mU, avP, A, bAF, Rj, lb, atf, yq, afj, dC, hL, tl, GN, B, dA, mr, aDH, ti, aNG, v, V, GS, qH, zT, DK, rS, bza, aql, MI, rD, bSH, aKG, ae, jf, ev, asy, aGp, bu, t, bSM, cl, kP, wO, lN, au, bSv, gX, Gg, zu, yv, vi, nZ, hl, pY, ml, vG, eq, aoz, hd, ds, hD, vc, Dj, yr, d, aVd, Z, adj, dr, gk, akQ, pP, ll, atg, gZ, Km, y, aXu, aC, bzx, al, gf, bSt, xE, u, pX, M, K, bLj, dF, eL, ago, bbC, lW, aNb, adE, aB, g, iP, wT, bAh, ou, avS, AM, U, Hb, m, Uw, abX, lV, zX, bSp, aw, NT, fg, JW, bKr, ft, mh, nV, bSG, uV, ZN, dx, RE, bSu, aev, bSS, jT, aHr, JP, ar, kT, GG, W, anZ, bo, yP, mg, ZG, it, ZV, wj, fG, jx, qQ, wo, mS, hz, j, sl, J, er, jF, MR, bBN, qG, Fg, kl, Y, lW, LO, r, bSy, wA, Qi, bmS, c, kB, hc, aNJ, dh, JL, cS, bSr, dy, UF, gT, gx, kh, aZ, bs, bS, pl, EA, hF, vN, dP, fU, sN, aet, UG, oj, vB, tt, bKm, aOh, vl, uX, IR, xs, eH, cr, bSn, hp, iR, Bu, agn, qa, bJt, dR, pu, bib, cP, bC, ei, sT, hE, xg, bSw, st, Fj, pf, eb, amj, aoM, aR, ata, il, iX, qM, de, H, bhm, pk, hm, tw, aW, aeC, hJ, avx, eM, wB, aYS, R, aFc, ko, bKx, fr, ct, qN, vY, rc, biN, bSq, cc, Mm, bAi, PD, h, jy, dL, aKE, mY, vx, Dg, asp, aq, kp, Hu, kk, mb, op, lx, mX, arf, JR, adZ, Uv, HR, bSB, bL, eR, sM, cR, tc, asA, C, so, Ns, Zp, mj, GL, uT, qp, aT, bSx, bK, af, ak, bSI, s, iU, oV, bAv, bSm, hf, OC, BU, sS, nM, aGo, fP, mf, aoJ, Kd, bSF, ha, vr, jE, tK, qC, nH, l, F, xl, oz, mW, s, ts, fb, iL, yF, qt, yy, gl, af, ek, LB, es, mO, bE, arC, awk, aS, pk); #c1(INSL3) c2(NP_001252516) c3(insulin-like 3 isoform 1 precursor) c4(94051) c5(94052, 94053, 94054, 94055, 94056, 94057) c6( ) c7(gU, aA, bLf, hz, aj, eb, UI, AO, J, UE, ft, hd, bSU, nv, uV, ae, bA, UF, yi, kl, s, ND, aeC, Lf, aF, bu, t, nB, PI, Uw); #c1(INSL3) c2(NP_005534) c3(insulin-like 3 isoform 2 precursor) c4(157220) c5(157221, 157222, 157223, 157224, 157225, 157226) c6( ) c7(gU, aA, bLf, hz, aj, eb, UI, AO, J, UE, ft, hd, bSU, nv, uV, ae, bA, UF, yi, kl, s, ND, aeC, Lf, aF, bu, t, nB, PI, Uw); #c1(INSL4) c2(NP_002186) c3(early placenta insulin-like peptide precursor) c4(126387) c5(126388, 126389, 126390, 126391, 126392, 126393) c6( ) c7(f, nJ, CN, iV, aT); #c1(INSL6) c2(NP_009110) c3(insulin-like peptide INSL6 precursor) c4(170743) c5(170744, 170745, 170746, 170747, 170748, 170749) c6( ) c7(eG, iV); #c1(INSM1) c2(NP_002187) c3(insulinoma-associated protein 1) c4(126394) c5(126395, 126396, 126397, 126398, 126399, 126400, 126401) c6( ) c7(jf, T, ft, d, bP, pX, aq, cm, eW); #c1(INSM2) c2(NP_115983) c3(insulinoma-associated protein 2) c4(225103) c5(225104, 225105, 225106, 225107, 225108, 225109, 225110, 225111) c6( ) c7(aq, aj, A, J, bo); #c1(INSR) c2(NP_000199) c3(insulin receptor isoform Long preproprotein) c4(4450) c5(4451, 4452, 4453, 4454, 4455, 4456) c6( ) c7(T, ea, ur, ci, Ef, bv, aE, dv, bJj, K, qM, wt, de, pk, aa, azu, Hd, aW, fc, hl, uY, g, uC, GR, Qt, ou, eM, go, U, cj, m, R, aCo, dD, aw, aj, aje, JW, El, bSV, KN, jy, dL, uV, A, asp, atf, aq, JR, bJ, iO, kT, dB, bo, hL, mg, at, bSB, bSX, lB, B, bL, eR, oe, cz, v, j, avD, bSY, q, J, qH, Ql, eA, aPW, mj, uT, bSW, aT, bq, an, ae, af, ak, kl, s, r, en, cl, t, pa, JL, mf, np, dy, gT, bt, rA, hl, aZ, jh, aCm, dG, oH, uu, eq, ad, dP, QJ, oj, nJ, ff, xs, bSn, dq, bz, qa, dR, ei); #c1(INSR) c2(NP_001073285) c3(insulin receptor isoform Short preproprotein) c4(35741, 35742) c5(35743, 35744, 35745, 35746, 35747, 35748, 35749) c6( ) c7(T, ea, ur, ci, Ef, bv, aE, dv, bJj, K, qM, wt, dQ, pk, aa, azu, Hd, aW, fc, hl, uY, g, uC, GR, Qt, ou, eM, go, U, cj, m, R, aCo, dD, aw, aj, aje, JW, El, bSV, KN, jy, dL, uV, A, asp, atf, aq, JR, bJ, iO, kT, dB, bo, hl, mg, at, bSB, bSX, lB, B, bL, eR, oe, cz, v, j, avD, bSY, q, J, qH, QI, eA, aPW, mj, uT, bSW, aT, bq, an, ae, af, ak, kl, s, r, en, cl, t, pa, JL, mf, np, dy, gT, bt, rA, hl, aZ, jh, aCm, dG, oH, uu, eq, ad, dP, QJ, oj, nJ, ff, xs, bSn, dq, bz, qa, dR, ei); #c1(INSR) c2(XP_011526290) c3(insulin receptor isoform X1) c4( ) c5(320554, 320555, 320556, 320557, 320558) c6( ) c7(T, ea, ur, ci, Ef, bv, aE, dv, bJj, K, qM, wt, de, pk, aa, azu, Hd, aW, fc, hl, uY, g, uC, GR, Qt, ou, eM, go, U, cj, m, R, aCo, dD, aw, aj, aje, JW, El, bSV, KN, jy, dL, uV, A, asp, atf, aq, JR, bJ, iO, kT, dB, bo, hL, mg, at, bSB, bSX, lB, B, bL, eR, oe, cz, v, j, avO, bSY, q, J, qH, QI, eA, aPW, mj, uT, bSW, aT, bq, an, ae, af, ak, kl, s, r, en, cl, t, pa, JL, mf, np, dy, gT, bt, rA, hl, aZ, jh, aCm, dG, oH, uu, eq, ad, dP, QJ, oj, nJ, ff, xs, bSn, dq, bz, qa, dR, ei); #c1(INSRR) c2(NP_055030) c3(insulin receptor-related protein precursor) c4(178396) c5(178397, 178398, 178399, 178400, 178401, 178402) c6( ) c7(bL, ck, bE, aW, e, qK, dV, aT, bB, bjL, h, fr, aoD, aot, cm, eW); #c1(INTS1) c2(NP_001073922) c3(integrator complex subunit 1) c4(36553, 36554) c5(36555, 36556, 36557, 36558, 36559, 36560, 36561, 36562, 36563, 36564, 36565, 36566) c6( ) c7(T, h, n, fr, bw, bt, QA); #c1(INTS(2) c2(NP_001135943) c3(integrator complex subunit 12) c4(53631, 53632, 53633, 53634, 53635) c5(53636, 53637, 53638, 53639, 53640, 53641, 53642, 53643, 53644, 53645, 53646, 53647, 53648, 53649, 53650, 53651, 53652, 53653, 53654, 53655, 53656, 53657, 53658, 53659, 53660, 53661, 53662, 53663, 53664, 53665, 53666, 53667, 53668, 53669, 53670, 53671, 53672, 53673, 53674, 53675, 53676) c6( ) c7(at, Dp, cz, aa); #c1(INTS2) c2(NP_065799) c3(integrator complex subunit 2) c4(205540, 205541) c5(205542, 205543, 205544, 205545, 205546, 205547, 205548, 205549, 205550, 205551, 205552, 205553) c6( ) c7(btc, T, dA, pY, fZ, Hn, n, ci, oc, q, er, lg, aT, h, dl, hu, avt, Cy, Yi, ia, OV, vT, aW, Y, ff, yq, en, hf, cl, t, g, ik, bw, ck, AM, hL, m, bt, it, B); #c1(INTS3) c2(NP_075391) c3(integrator complex subunit 3) c4(213844) c5(213845, 213846, 213847, 213848, 213849, 213850, 213851, 213852, 213853, 213854, 213855, 213856, 213857) c6( ) c7(ck, pg); #c1(INTS3) c2(XP_005276792) c3(integrator complex subunit 3 isoform X1) c4(285711) c5(285712) c6( ) c7(ck, pg); #c1(INTS3) c2(XP_011545224) c3(integrator complex subunit 3 isoform X2) c4( ) c5(344000, 344001, 344002, 344003, 344004, 344005, 344006, 344007, 344008) c6( ) c7(ck, pg); #c1(INTS4) c2(NP_291025) c3(integrator complex subunit 4) c4(228888, 228889) c5(228890, 228891, 228892, 228893, 228894, 228895, 228896, 228897, 228898, 228899, 228900, 228901) c6( ) c7(AW, c, ig, Ny, bKk, oW, CH, pb, aa); #c1(INTS4) c2(XP_005274450) c3(integrator complex subunit 4 isoform X2) c4(285082) c5( ) c6( ) c7(AW, c, ig, Ny, bKk, oW, CH, pb, aa); #c1(INTS4) c2(XP_011543654) c3(integrator complex subunit 4 isoform X1) c4( ) c5(342765, 342766, 342767, 342768, 342769, 342770) c6( ) c7(AW, c, ig, Ny, bKk, oW, CH, pb, aa); #c1(INTS5) c2(NP_085131) c3(integrator complex subunit 5) c4(219352) c5(219353, 219354, 219355, 219356, 219357, 219358) c6( ) c7(aW, aT); #c1(INTS6) c2(NP_001035026) c3(integrator complex subunit 6 isoform b) c4(32450, 32451) c5(32452, 32453, 32454, 32455, 32456, 32457, 32458, 32459, 32460, 32461, 32462, 32463, 32464, 32465, 32466, 32467) c6( ) c7(T, bP, eM, aW, eu, al, cm, ci, t, ae, X, ad); #c1(INTS6) c2(NP_001035027) c3(integrator complex subunit 6 isoform c) c4(32468) c5(32469, 32470, 32471, 32472, 32473, 32474) c6( ) c7(T, bP, eM, aW, eu, aT, cm, ci, t, ae, X, ad); #c1(INTS6) c2(NP_036273) c3(integrator complex subunit 6 isoform a) c4(172782, 172783, 172784, 172785, 172786) c5(172787, 172788, 172789, 172790, 172791, 172792, 172793, 172794, 172795, 172796, 172797, 172798, 172799, 172800, 172801) c6( ) c7(T, bP, eM, aW, eu, aT, cm, ci, t, ae, X, ad); #c1(INTS7) c2(NP_001186738) c3(integrator complex subunit 7 isoform 4) c4(78468) c5(78469, 78470, 78471, 78472, 78473, 78474) c6( ) c7(bk, ac); #c1(INTS7) c2(NP_001186740) c3(integrator complex subunit 7 isoform 2) c4(78475) c5(78476, 78477, 78478, 78479, 78480, 78481) c6( ) c7(bk, ac); #c1(INTS7) c2(NP_001186741) c3(integrator complex subunit 7 isoform 3) c4(78482) c5(78483, 78484, 78485, 78486, 78487, 78488) c6( ) c7(bK, ac); #c1(INTS7) c2(NP_056249) c3(integrator complex subunit 7 isoform 1) c4(186720) c5(186721, 186722, 186723, 186724, 186725, 186726) c6( ) c7(bK, ac); #c1(INTS8) c2(NP_060334) c3(integrator complex subunit 8) c4(195435, 195436) c5(195437, 195438, 195439, 195440, 195441, 195442, 195443, 195444, 195445, 195446, 195447, 195448) c6( ) c7(Jj, aGd, BG, ok); #c1(INTU) c2(NP_056508) c3(protein inturned) c4(187844) c5(187845, 187846, 187847, 187848, 187849, 187850) c6( ) c7(ck, dA, aW, aT, h, ci, bC, g, eA); #c1(INTU) c2(XP_011530151) c3(protein inturned isoform X6) c4(326043) c5( ) c6( ) c7(ck, dA, aW, aT, h, ci, bC, g, eA); #c1(INTU) c2(XP_011530153) c3(protein inturned isoform X8) c4( ) c5(326044, 326045, 326046, 326047, 326048, 326049) c6( ) c7(ck, dA, aW, aT, h, ci, bC, g, eA); #c1(INVS) c2(NP_055240) c3(inversin isoform a) c4(180004) c5(180005, 180006, 180007, 180008, 180009, 180010) c6( ) c7(aKZ, ck, Mg, ago, eR, BV, bKt, cr, NI, vm, bSZ, Hb, Fq, Tp, aY, bt, vN, nC); #c1(INVS) c2(XP_011516834) c3(inversin isoform X1) c4(310268) c5(310269) c6( ) c7(aKZ, ck, Mg, ago, eR, BV, bKt, cr, NI, vm, bSZ, Hb, Fq, Tp, aY, bt, vN, nC); #c1(INVS) c2(XP_011516835) c3(inversin isoform X2) c4(310270) c5(310271, 310272, 310273, 310274, 310275, 310276) c6( ) c7(aKZ, ck, Mg, ago, eR, BV, bKt, cr, NI, vm, bSZ, Hb, Fq, Tp, aY, bt, vN, nC); #c1(INVS) c2(XP_011516840) c3(inversin isoform X3) c4( ) c5(310277, 310278, 310279, 310280, 310281) c6( ) c7(akZ, ck, Mg, ago, eR, BV, bKt, cr, NI, vm, bSZ, Hb, Fq, Tp, aY, bt, vN, nC); #c1(INVS) c2(XP_011516844) c3(inversin isoform X5) c4(310282, 310283) c5(310284, 310285, 310286, 310287, 310288, 310289) c6( ) c7(aKZ, ck, Mg, ago, eR, BV, bKt, cr, NI, vm, bSZ, Hb, Fq, Tp, aY, bt, vN, nC); #c1(IP6K1) c2(NP_001006115) c3(inositol hexakisphosphate kinase 1 isoform 2) c4( ) c5(22180, 22181, 22182, 22183, 22184, 22185, 22186, 22187) c6( ) c7(bL); #c1(IP6K1) c2(NP_001229758) c3(inositol hexakisphosphate kinase 1 isoform 1) c4(85160, 85161) c5(85162, 85163, 85164, 85165, 85166, 85167, 85168, 85169) c6( ) c7(bL); #c1(IP6K2) c2(NP_001005911) c3(inositol hexakisphosphate kinase 2 isoform b) c4(22145) c5(22146, 22147, 22148, 22149, 22150, 22151, 22152, 22153) c6( ) c7(ci, T, np, m, rR); #c1(IP6K2) c2(NP_001177245) c3(inositol hexakisphosphate kinase 2 isoform d) c4(72536) c5(72537, 72538, 72539, 72540, 72541, 72542, 72543, 72544) c6( ) c7(ci, T, np, m, rR); #c1(IP6K2) c2(NP_001177246) c3(inositol hexakisphosphate kinase 2 isoform e) c4(72545) c5(72546, 72547, 72548, 72549, 72550, 72551) c6( ) c7(ci, T, np, m, rR); #c1(IP6K2) c2(NP_057375) c3(inositol hexakisphosphate kinase 2 isoform a) c4(190456) c5(190457, 190458, 190459, 190460, 190461) c6( ) c7(ci, T, np, m, rR); #c1(IP6K2) c2(XP_006713264) c3(inositol hexakisphosphate kinase 2 isoform X4) c4( ) c5(287065) c6( ) c7(ci, T, np, m, rR); #c1(IP6K2) c2(XP_011532123) c3(inositol hexakisphosphate kinase 2 isoform X7) c4(328162) c5(328163) c6( ) c7(ci, T, np, m, rR); #c1(IP6K2) c2(XP_011532125) c3(inositol hexakisphosphate kinase 2 isoform X1) c4( ) c5(328164, 328165, 328166, 328167, 328168) c6( ) c7(ci, T, np, m, rR); #c1(IP6K3) c2(NP_001136355) c3(inositol hexakisphosphate kinase 3) c4(54841) c5(54842, 54843, 54844, 54845, 54846, 54847, 54848, 54849, 54850, 54851) c6( ) c7(aw, eZ); #c1(IPCEF1) c2(XP_005266979) c3(interactor protein for cytohesin exchange factors 1 isoform X1) c4(280571) c5(280572, 280573, 280574, 280575, 280576, 280577, 280578, 280579, 280580, 280581) c6( ) c7(aj, hn, fJ); #c1(IPCEF1) c2(XP_005266980) c3(interactor protein for cytohesin exchange factors 1 isoform X2) c4(280582, 280583) c5(280584, 280585, 280586, 280587, 280588, 280589, 280590, 280591, 280592, 280593, 280594, 280595) c6( ) c7(aj, hn, fJ); #c1(IPMK) c2(NP_689416) c3(inositol polyphosphate multikinase) c4(242703) c5(242704, 242705, 242706, 242707, 242708, 242709) c6( ) c7(aw); #c1(IPO11) c2(NP_001128251) c3(importin-Il isoform 1) c4(49799, 49800, 49801, 49802, 49803, 49804) c5(49805, 49806, 49807, 49808, 49809, 49810, 49811, 49812, 49813, 49814) c6( ) c7(aoU); #c1(IPO11) c2(NP_057422) c3(importin-11 isoform 2) c4( ) c5(190712, 190713, 190714, 190715, 190716, 190717, 190718, 190719, 190720, 190721, 190722, 190723, 190724, 190725, 190726, 190727, 190728, 190729, 190730, 190731, 190732, 190733, 190734, 190735, 190736, 190737) c6( ) c7(aoU); #c1(IPO13) c2(XP_011540763) c3(importin-13 isoform X1) c4(339333) c5(339334, 339335, 339336, 339337, 339338, 339339) c6( ) c7(bM, sp, T, aT, pM); #c1(IPO7) c2(NP_006382) c3(importin-7) c4(164518) c5(164519, 164520, 164521, 164522, 164523, 164524) c6( ) c7(h, lo, ae, ey, cz); #c1(IPO8) c2(NP_001177924) c3(importin-8 isoform 2) c4(73173) c5(73174, 73175, 73176, 73177, 73178, 73179) c6( ) c7(fe); #c1(IPO8) c2(NP_006381) c3(importin-8 isoform 1) c4(164505) c5(164506, 164507, 164508, 164509, 164510, 164511, 164512, 164513, 164514, 164515, 164516, 164517) c6( ) c7(fe); #c1(IPO8) c2(XP_011518849) c3(importin-8 isoform X2) c4(313203) c5( ) c6( ) c7(fe); #c1(IPO9) c2(NP_060555) c3(importin-9) c4(196539) c5(196540, 196541, 196542, 196543, 196544, 196545) c6( ) c7(af); #c1(IPO9) c2(XP_011508032) c3(importin-9 isoform X1) c4(298488) c5(298489, 298490, 298491, 298492, 298493, 298494) c6( ) c7(af); #c1(IQCB1) c2(NP_001018864) c3(14 calmodulin-binding motif-containing protein 1 isoform a) c4(27556) c5(27557, 27558, 27559, 27560, 27561, 27562) c6( ) c7(bTb, vJ, gX, aA, vm, nw, dy, Nh, bTa, Sp, nC, xt, aY, UF); #c1(IQCB1) c2(NP_001018865) c3(14 calmodulin-binding motif-containing protein 1 isoform c) c4(27563) c5(27564, 27565, 27566, 27567, 27568, 27569) c6( ) c7(bTb, vJ, gX, aA, vm, nw, dy, Nh, bTa, Sp, nC, xt, aY, UF); #c1(IQCB1) c2(XP_005247968) c3(IQ calmodulin-binding motif-containing protein 1 isoform X1) c4(268156) c5( ) c6( ) c7(bTb, vJ, gX, aA, vm, nw, dy, Nh, bTa, Sp, nC, xt, aY, UF); #c1(IQCB1) c2(XP_005247970) c3(IQ calmodulin-binding motif-containing protein 1 isoform X3) c4( ) c5(268157, 268158, 268159, 268160, 268161, 268162) c6( ) c7(bTb, vJ, gX, aA, vm, nw, dy, Nh, bTa, Sp, nC, xt, aY, UF); #c1(IQCG) c2(NP_001127907) c3(IQ domain-containing protein G) c4(49494, 49495) c5(49496, 49497, 49498, 49499, 49500, 49501, 49502, 49503, 49504, 49505, 49506, 49507, 49508, 49509, 49510, 49511) c6( ) c7(IT); #c1(IQCH) c2(NP_001026885) c3(IQ domain-containing protein H isoform 1) c4(29837) c5(29838, 29839, 29840, 29841, 29842, 29843) c6( ) c7(Y); #c1(IQCH) c2(NP_001271277) c3(IQ domain-containing protein H isoform 4) c4(103813) c5(103814, 103815, 103816, 103817, 103818, 103819) c6( ) c7(Y); #c1(IQCH) c2(NP_073621) c3(IQ domain-containing protein H isoform 2) c4( ) c5(213180, 213181, 213182, 213183, 213184) c6( ) c7(Y); #c1(IQCH) c2(XP_011520214) c3(IQ domain-containing protein H isoform X6) c4(315082, 315083) c5(315084, 315085, 315086, 315087, 315088, 315089, 315090, 315091, 315092, 315093, 315094, 315095) c6( ) c7(Y); #c1(IQCH) c2(XP_011520217) c3(IQ domain-containing protein H isoform X9) c4(315096) c5( ) c6( ) c7(Y); #c1(IQCH) c2(XP_011520225) c3(IQ domain-containing protein H isoform X16) c4( ) c5(315097) c6( ) c7(Y); #c1(IQCJ) c2(NP_001036170) c3(IQ domain-containing protein J isoform CaMBPvl) c4(34019) c5(34020, 34021, 34022, 34023, 34024, 34025) c6( ) c7(cm, eA); #c1(IQCJ) c2(NP_001036171) c3(IQ domain-containing protein J isoform CaMBPv2) c4(34026) c5(34027, 34028, 34029, 34030, 34031, 34032) c6( ) c7(cm, eA); #c1(IQCJ) c2(NP_001184029) c3(IQ domain-containing protein J isoform CaMBPv3) c4(75278) c5(75279, 75280, 75281, 75282, 75283, 75284) c6( ) c7(cm, eA); #c1(IQCJ-SCHIP1) c2(NP_001184042) c3(IQ motif containing J-schwannomin interacting protein I fusion protein isoform 1) c4(75319) c5(75320, 75321, 75322, 75323, 75324, 75325, 75326) c6( ) c7(bF, rQ, py, hp, rP); #c1(IQCJ-SCHIP1) c2(NP_001184043) c3(IQ motif containing J-schwannomin interacting protein 1 fusion protein isoform 2) c4(75327, 75328) c5(75329, 75330, 75331, 75332, 75333, 75334) c6( ) c7(bF, rQ, py, hp, rP); #c1(IQCK) c2(NP_694940) c3(IQ domain-containing protein K) c4(245444, 245445) c5(245446, 245447, 245448, 245449, 245450, 245451, 245452) c6( ) c7(v); #c1(IQCK) c2(XP_011544036) c3(IQ domain-containing protein K isoform X1) c4(343209) c5(343210, 343211, 343212, 343213, 343214) c6( ) c7(v); #c1(IQCK) c2(XP_011544038) c3(IQ domain-containing protein K isoform X3) c4( ) c5(343215, 343216, 343217, 343218, 343219) c6( ) c7(v); #c1(IQGAP1) c2(NP_003861) c3(ras GTPase-activating-like protein IQGAP1) c4(141581) c5(141582, 141583, 141584, 141585, 141586, 141587, 141588) c6( ) c7(T, aA, bB, ah, q, eA, hd, aT, lG, ey, K, aW, r, aF, g, t, fi, qJ, bF, nM, nB, bE, XO, m, it, B); #c1(IQGAP2) c2(NP_001272390) c3(ras GTPase-activating-like protein IQGAP2 isoform 3) c4(104054) c5(104055, 104056, 104057, 104058, 104059, 104060) c6( ) c7(aq, fq, B, aF, aA); #c1(IQGAP2) c2(NP_001272391) c3(ras GTPase-activating-like protein IQGAP2 isoform 4) c4(104061) c5( ) c6( ) c7(aq, fq, B, aF, aA); #c1(IQGAP2) c2(NP_006624) c3(ras GTPase-activating-like protein IQGAP2 isoform 1) c4(166706) c5(166707, 166708, 166709, 166710, 166711, 166712) c6( ) c7(aq, fq, B, aF, aA); #c1(IQGAP2) c2(XP_005248471) c3(ras GTPase-activating-like protein IQGAP2 isoform X3) c4(268404) c5(268405, 268406, 268407, 268408, 268409, 268410) c6( ) c7(aq, fq, B, aF, aA); #c1(IQGAP2) c2(XP_011541409) c3(ras GTPase-activating-like protein IQGAP2 isoform X2) c4( ) c5(340010, 340011, 340012, 340013, 340014, 340015) c6( ) c7(aq, fq, B, aF, aA); #c1(IQGAP3) c2(NP_839943) c3(ras GTPase-activating-like protein IQGAP3) c4(253570) c5(253571, 253572, 253573, 253574, 253575, 253576) c6( ) c7(T, bz); #c1(IQSEC1) c2(NP_001127854) c3(IQ motif and SEC7 domain-containing protein 1 isoform a) c4(49397, 49398) c5(49399, 49400, 49401, 49402, 49403, 49404, 49405, 49406, 49407, 49408, 49409, 49410) c6( ) c7(iu, T, aE, aW, aT, cy, aq, g, AD, B, us); #c1(IQSEC1) c2(NP_055684) c3(IQ motif and SEC7 domain-containing protein 1 isoform b) c4(182761) c5(182762, 182763, 182764, 182765, 182766, 182767) c6( ) c7(iu, T, aE, aW, al, cy, aq, g, AD, B, us); #c1(IQSEC1) c2(XP_011532612) c3(IQ motif and SEC7 domain-containing protein 1 isoform X7) c4(328711) c5(328712, 328713, 328714, 328715, 328716, 328717) c6( ) c7(iu, T, aE, aW, aT, cy, aq, g, AD, B, us); #c1(IQSEC1) c2(XP_011532616) c3(IQ motif and SEC7 domain-containing protein 1 isoform X8) c4( ) c5(328718) c6( ) c7(iu, T, aE, aW, aT, cy, aq, g, AO, B, us); #c1(IQSEC1) c2(XP_011532617) c3(IQ motif and SEC7 domain-containing protein 1 isoform X9) c4( ) c5(328719, 328720) c6( ) c7(iu, T, aE, aW, aT, cy, aq, g, AO, B, us); #c1(IQSEC2) c2(NP_001104595) c3(IQ motif and SEC7 domain-containing protein 2 isoform 1) c4(42207) c5(42208, 42209, 42210, 42211, 42212, 42213, 42214, 42215, 42216, 42217, 42218, 42219) c6( ) c7(nd, nf, afg, nA, bU, WI, eA, aR); #c1(IQSEC2) c2(NP_055890) c3(IQ motif and SEC7 domain-containing protein 2 isoform 2) c4(184210) c5(184211, 184212, 184213, 184214, 184215, 184216) c6( ) c7(nd, nf, afg, nA, bU, WI, eA, aR); #c1(IQSEC2) c2(XP_006724643) c3(IQ motif and SEC7 domain-containing protein 2 isoform X2) c4(296161) c5( ) c6( ) c7(nd, nf, afg, nA, bU, WI, eA, aR); #c1(IQSEC2) c2(XP_011529075) c3(IQ motif and SEC7 domain-containing protein 2 isoform X4) c4( ) c5(324447) c6( ) c7(nd, nf, afg, nA, bU, WI, eA, aR); #c1(IQSEC3) c2(NP_001164209) c3(IQ motif and SEC7 domain-containing protein 3 isoform 1) c4(66804) c5(66805, 66806, 66807, 66808, 66809, 66810, 66811, 66812, 66813, 66814) c6( ) c7(cd); #c1(IQSEC3) c2(NP_056047) c3(IQ motif and SEC7 domain-containing protein 3 isoform 2) c4(185281) c5( ) c6( ) c7(cd); #c1(IQSEC3) c2(XP_011519262) c3(IQ motif and SEC7 domain-containing protein 3 isoform X3) c4( ) c5(313914, 313915, 313916, 313917, 313918, 313919) c6( ) c7(cd); #c1(IRAK1) c2(NP_001020413) c3(interleukin-1 receptor-associated kinase 1 isoform 2) c4(28739) c5(28740, 28741, 28742, 28743, 28744, 28745) c6( ) c7(bL, bM, DM, dD, ea, fG, aA, aj, j, ci, C, eq, J, ad, wD, aE, Bc, h, cy, dl, ae, eL, eu, Of, eZ, Og, aF, t, pb, ek, ac, nB, m, y, Pr); #c1(IRAK1) c2(NP_001020414) c3(interleukin-1 receptor-associated kinase 1 isoform 3) c4(28746) c5(28747, 28748, 28749, 28750, 28751, 28752) c6( ) c7(bL, bM, DM, dD, ea, fG, aA, aj, j, ci, C, eq, J, ad, wD, aE, Bc, h, cy, dl, ae, eL, eu, Of, eZ, Og, aF, t, pb, ek, ac, nB, m, y, Pr); #c1(IRAK1) c2(NP_001560) c3(interleukin-1 receptor-associated kinase 1 isoform 1) c4(118973) c5(118974, 118975, 118976, 118977, 118978, 118979) c6( ) c7(bL, bM, DM, dD, ea, fG, aA, aj, j, ci, C, eq, J, ad, wD, aE, Bc, h, cy, dL, ae, eL, eu, Of, eZ, Og, aF, t, pb, ek, ac, nB, m, y, Pr); #c1(IRAK1) c2(XP_005274725) c3(interleukin-1 receptor-associated kinase 1 isoform X1) c4(285338, 285339) c5(285340, 285341, 285342, 285343, 285344, 285345, 285346, 285347, 285348, 285349, 285350, 285351) c6( ) c7(bL, bM, DM, dD, ea, fG, aA, aj, j, ci, C, eq, J, ad, wD, aE, Bc, h, cy, dl, ae, eL, eu, Of, eZ, Og, aF, t, pb, ek, ac, nB, m, y, Pr); #c1(IRAK1BP1) c2(NP_001010844) c3(interleukin-1 receptor-associated kinase 1-binding protein 1) c4(23824) c5(23825, 23826, 23827, 23828, 23829, 23830) c6( ) c7(IN); #c1(IRAK2) c2(NP_001561) c3(interleukin-1 receptor-associated kinase-like 2) c4(118980) c5(118981, 118982, 118983, 118984, 118985, 118986) c6( ) c7(oO, r, h, dz, q, pm, bTc); #c1(IRAK3) c2(NP_001135995) c3(interleukin-1 receptor-associated kinase 3 isoform b) c4(53936) c5(53937, 53938, 53939, 53940, 53941, 53942) c6( ) c7(oO, bM, lk, jx, v, C, eA, aDF, av, aT, K, jo, bA, d, iV, bTd, axp, bz, bTe, fD, ei, y, iu); #c1(IRAK3) c2(NP_009130) c3(interleukin-1 receptor-associated kinase 3 isoform a) c4(170963) c5(170964, 170965, 170966, 170967, 170968, 170969) c6( ) c7(oO, bM, lk, jx, v, C, eA, aDF, av, aT, K, jo, bA, d, iV, bTd, axp, bz, bTe, fD, ei, y, iu); #c1(IRAK4) c2(NP_001138728) c3(interleukin-1 receptor-associated kinase 4 isoform b) c4(57461) c5(57462, 57463, 57464, 57465, 57466, 57467, 57468, 57469) c6( ) c7(oO, bM, Dw, dD, fR, ea, eq, C, eA, ad, eW, dV, Os, h, cy, Ds, bTf, ae, eu, bNq, mQ, eZ, awv, ajz, zV, cj, aNn, y, Tr, zA, bTc); #c1(IRAK4) c2(NP_057207) c3(interleukin-1 receptor-associated kinase 4 isoform a) c4(189496, 189497, 189498) c5(189499, 189500, 189501, 189502, 189503, 189504, 189505, 189506, 189507, 189508, 189509, 189510, 189511, 189512, 189513, 189514, 189515, 189516, 189517, 189518, 189519, 189520, 189521, 189522) c6( ) c7(oO, bM, Dw, dD, fR, ea, eq, C, eA, ad, eW, dV, Os, h, cy, Ds, bTf, ae, eu, bNq, md, eZ, aw, ajz, zV, cj, aNn, y, Tr, zA, bTc); #c1(IREB2) c2(NP_004127) c3(iron-responsive element-binding protein 2) c4(143797) c5(143798, 143799, 143800, 143801, 143802, 143803, 143804) c6( ) c7(iu, T, EM, am, af, yt, oW, yy, yu, at, xw, bz, B, N); #c1(IRF1) c2(NP_002189) c3(interferon regulatory factor I) c4(126434, 126435) c5(126436, 126437, 126438, 126439, 126440, 126441, 126442, 126443, 126444, 126445, 126446, 126447, 126448, 126449, 126450, (26451, 126452) c6( ) c7(bM, T, FR, Js, pf, ea, if, qx, fd, bB, cL, ci, x, dV, Ds, K, hH, aa, JI, aW, mQ, cm, bw, ba, eM, iT, mx, aiM, k, m, eg, cZ, dD, n, fr, qN, pM, cd, br, ai, Nh, cU, lG, h, dl, dd, sp, aq, mo, hL, hj, at, rx, eo, B, Jo, Dw, jx, aA, cR, j, V, o, aT, aHs, j, jf, qw, iU, Y, bg, lo, en, hf, cl, t, f, ck, cT, sS, ac, gX, rk, bt, aCA, btY, bs, wV, eq, jE, hF, hX, av, F, Ot, hD, pq, qZ, qt, aff, ff, gK, cr, aF, hp, iR, bF, bE, z, aEN, bC, ei, y); #c1(IRF2) c2(NP_002190) c3(interferon regulatory factor 2) c4(126453, 126454, 126455) c5(126456, 126457, 126458, 126459, 126460, 126461, 126462) c6( ) c7(gV, T, bM, aC, aw, ht, jx, NT, bB, n, jE, aFv, eW, hX, F, aE, Rq, apJ, aT, cy, bK, lQ, DU, hu, pq, jf, iU, aW, sp, cr, lo, eE, t, cm, ck, bE, bNF, cj, m, bt, B); #c1(IRF2) c2(XP_005263041) c3(interferon regulatory factor 2 isoform X2) c4(278234) c5(278235, 278236, 278237, 278238, 278239, 278240, 278241, 278242, 278243, 278244, 278245, 278246) c6( ) c7(gV, T, bM, aC, aw, ht, jx, NT, bB, n, jE, aFv, eW, hX, F, aE, Rq, apJ, aT, cy, bK, lQ, DU, hu, pq, jf, iU, aW, sp, cr, lo, eE, t, cm, ck, bE, bNF, cj, m, bt, B); #c1(IRF2) c2(XP_011530231) c3(interferon regulatory factor 2 isoform X3) c4( ) c5(326126, 326127, 326128, 326129, 326130, 326131, 326132, 326133, 326134, 326135) c6( ) c7(gV, T, bM, aC, aw, ht, jx, NT, bB, n, jE, aFv, eW, hX, F, aE, Rq, apJ, aT, cy, bk, lQ, DU, hu, pq, jf, iU, aW, sp, cr, lo, eE, t, cm, ck, bE, bNF, cj, m, bt, B); #c1(IRF2BP2) c2(NP_001070865) c3(interferon regulatory factor 2-binding protein 2 isoform B) c4(34854) c5(34855, 34856, 34857, 34858, 34859, 34860, 34861, 34862, 34863, 34864) c6( ) c7(apy, cy); #c1(IRF2BP2) c2(NP_892017) c3(interferon regulatory factor 2-binding protein 2 isoform A) c4(257618) c5(257619, 257620, 257621, 257622, 257623, 257624) c6( ) c7(apy, cy); #c1(IRF2BPL) c2(NP_078772) c3(interferon regulatory factor 2-binding protein-like) c4(215418) c5(215419, 215420, 215421, 215422, 215423, 215424) c6( ) c7(cm, asP, aan); #c1(IRF3) c2(NP_001184052) c3(interferon regulatory factor 3 isoform 3) c4(75335) c5(75336, 75337) c6( ) c7(gV, T, bM, Dw, QO, FR, Ke, if, al, jx, NT, jh, bB, wV, v, EA, qN, eA, eW, F, ds, dV, cU, aAn, Ds, dL, DU, h, afu, Nr, Y, wc, apn, eZ, fc, aiT, dq, ls, lo, uF, Fq, CA, yq, po, Nq, gk, ac, Ui, eM, bE, dc, bNF, aiM, at, wq, nw, cP, y, bt, B); #c1(IRF3) c2(NP_001184053) c3(interferon regulatory factor 3 isoform 4) c4(75338, 75339, 75340) c5(75341, 75342, 75343, 75344, 75345, 75346, 75347, 75348, 75349, 75350, 75351, 75352, 75353, 75354, 75355, 75356, 75357, 75358) c6( ) c7(gV, T, bM, Dw, QO, FR, Ke, if, al, jx, NT, jh, bB, wV, v, EA, qN, eA, eW, F, ds, dV, cU, aAn, Ds, dL, DU, h, afu, Nr, Y, wc, apn, eZ, fc, aiT, dq, ls, lo, uF, Fq, CA, yq, po, Nq, gk, ac, Ui, eM, bE, dc, bNF, aiM, at, wq, nw, cP, y, bt, B); #c1(IRF3) c2(NP_001184055) c3(interferon regulatory factor 3 isoform 5) c4( ) c5(75359, 75360, 75361, 75362, 75363, 75364, 75365, 75366) c6( ) c7(gV, T, bM, Dw, QO, fR, Ke, if, al, jx, NT, jh, bB, wV, v, EA, qN, eA, eW, F, ds, dV, cU, aAn, Ds, dL, DU, h, afu, Nr, Y, wc, apn, eZ, fc, aiT, dq, ls, lo, uF, Fq, CA, yq, po, Nq, gk, ac, Ui, eM, bE, dc, bNF, aiM, at, wq, nw, cP, y, bt, B); #c1(IRF3) c2(NP_001184057) c3(interferon regulatory factor 3 isoform 6) c4( ) c5(75367, 75368, 75369, 75370, 75371, 75372, 75373, 75374) c6( ) c7(gV, T, bM, Dw, QO, FR, Ke, if, al, jx, NT, jh, bB, wV, v, EA, qN, eA, eW, F, ds, dV, cU, aAn, Ds, dL, DU, h, afu, Nr, Y, wc, apn, eZ, fc, aiT, dq, ls, lo, uF, Fq, CQ, yq, po, Nq, gk, ac, Ui, eM, bE, dc, bNF, aiM, at, wq, nw, cP, y, bt, B); #c1(IRF3) c2(XP_006723262) c3(interferon regulatory factor 3 isoform X1) c4(295053) c5(295054, 295055, 295056, 295057, 295058, 295059, 295060, 295061, 295062, 295063, 295064) c6( ) c7(gV, T, bM, Dw, QO, FR, Ke, if, al, jx, NT, jh, bB, wV, v, EA, qN, eA, eW, F, ds, dV, cU, aAn, Ds, dL, DU, h, afu, Nr, Y, wc, apn, eZ, fc, aiT, dq, ls, lo, uF, Fq, CQ, yq, po, Nq, gk, ac, Ui, eM, bE, dc, bNF, aiM, at, wq, nw, cP, y, bt, B); #c1(IRF4) c2(NP_001182215) c3(interferon regulatory factor 4 isoform 2) c4(74694) c5(74695, 74696, 74697, 74698, 74699, 74700) c6( ) c7(bM, T, fM, ajm, yk, iW, eX, NT, jh, aA, fd, n, oY, bX, hF, V, q, apQ, pe, Bj, afQ, Nn, KH, Ne, aT, cy, hn, rB, h, apD, eV, ji, pk, bTh, je, iU, xR, aW, Y, ff, Of, aph, r, fe, Og, lo, aF, hp, yq, en, hf, iV, cm, Cv, ck, cT, sS, iM, Dy, ac, TA, z, hk, cj, gn, ij, bTg, act, bt); #c1(IRF4) c2(NP_002451) c3(interferon regulatory factor 4 isoform 1) c4(128624, 128625) c5(128626, 128627, 128628, 128629, 128630, 128631, 128632) c6( ) c7(bM, T, fM, ajm, yk, iW, eX, NT, jh, aA, fd, n, oY, bX, hF, V, q, apQ, pe, Bj, afQ, Nn, KH, Ne, aT, cy, hn, rB, h, apD, eV, ji, pk, bTh, je, iU, xR, aW, Y, ff, Of, aph, r, fe, Og, lo, aF, hp, yq, en, hf, iV, cm, Cv, ck, cT, sS, iM, Dy, ac, TA, z, hk, cj, gn, ij, bTg, act, bt); #c1(IRF4) c2(XP_006715153) c3(interferon regulatory factor 4 isoform X1) c4(288806) c5(288807, 288808, 288809, 288810, 288811) c6( ) c7(bM, T, fM, ajm, yk, iW, eX, NT, jh, aA, fd, n, oY, bX, hF, V, q, apQ, pe, Bj, afQ, Nn, kH, Ne, aT, cy, hn, rB, h, apD, eV, ji, pk, bTh, je, iU, xR, aW, Y, ff, Of, aph, r, fe, Og, lo, aF, hp, yq, en, hf, iV, cm, Cv, ck, cT, sS, iM, Dy, ac, TA, z, hk, cj, gn, ij, bTg, act, bt); #c1(IRF5) c2(NP_001092097) c3(interferon regulatory factor 5 isoform b) c4(39340) c5(39341, 39342, 39343, 39344, 39345, 39346, 39347, 39348, 39349, 39350, 39351, 39352, 39353, 39354) c6( ) c7(Kd, T, dD, bM, Dw, fG, aw, bTi, fN, ez, bB, vr, cL, jh, j, eq, pz, AO, V, qW, mC, o, dP, hd, bW, qh, aT, aAn, dm, Ds, bxi, ey, lM, bc, bTj, hM, OV, A, iU, qt, aW, cr, eZ, fe, lo, yq, mo, pL, iV, bz, pb, acp, adz, bTk, ac, axs, nB, bE, hc, ajd, cj, dy, fL, eo, y, Pr, hv); #c1(IRF5) c2(NP_001229381) c3(interferon regulatory factor 5 isoform e) c4(84294, 84295, 84296, 84297) c5(84298, 84299, 84300, 84301, 84302, 84303, 84304, 84305, 84306, 84307, 84308, 84309, 84310, 84311, 84312, 84313, 84314, 84315, 84316, 84317, 84318, 84319, 84320, 84321, 84322, 84323, 84324, 84325, 84326, 84327, 84328, 84329, 84330, 84331, 84332, 84333, 84334, 84335, 84336, 84337, 84338) c6( ) c7(Kd, T, dD, bM, Dw, fG, aw, bTi, fN, ez, bB, vr, cL, jh, j, eq, pz, AD, V, qW, mC, o, dP, hd, bW, qh, aT, aAn, dm, Ds, bxi, ey, lM, bc, bTj, hM, OV, A, iU, qt, aW, cr, eZ, fe, lo, yq, mo, pL, iV, bz, pb, acp, adz, bTk, ac, axs, nB, bE, hc, ajd, cj, dy, fL, eo, y, Pr, hv); #c1(IRF5) c2(XP_011514462) c3(interferon regulatory factor 5 isoform X1) c4(307147, 307148, 307149) c5(307150, 307151, 307152, 307153, 307154, 307155) c6( ) c7(Kd, T, dD, bM, Dw, fG, aw, bTi, fN, ez, bB, vr, cL, jh, j, eq, pz, AO, V, qW, mC, o, dP, hd, bW, qh, aT, aAn, dm, Ds, bxi, ey, lM, bc, bTj, hM, OV, A, iU, qt, aW, cr, eZ, fe, lo, yq, mo, pL, iV, bz, pb, acp, adz, bTk, ac, axs, nB, bE, hc, ajd, cj, dy, fL, eo, y, Pr, hv); #c1(IRF5) c2(XP_011514463) c3(interferon regulatory factor 5 isoform X2) c4(307156) c5(307157) c6( ) c7(Kd, T, dD, bM, Dw, fG, aw, bTi, fN, ez, bB, vr, cL, jh, j, eq, pz, AO, V, qW, mC, o, dP, hd, bW, qh, aT, aAn, dm, Ds, bxi, ey, lM, bc, bTj, hM, OV, A, iU, qt, aW, cr, eZ, fe, lo, yq, mo, pL, iV, bz, pb, acp, adz, bTk, ac, axs, nB, bE, hc, ajd, cj, dy, fL, eo, y, Pr, hv); #c1(IRF5) c2(XP_011514466) c3(interferon regulatory factor 5 isoform X3) c4(307158) c5(307159, 307160, 307161, 307162, 307163, 307164, 307165) c6( ) c7(Kd, T, dD, bM, Dw, fG, aw, bTi, fN, ez, bB, vr, cL, jh, j, eq, pz, AO, V, qW, mC, o, dP, hd, bW, qh, aT, aAn, dm, Ds, bxi, ey, lM, bc, bTj, hM, OV, A, iU, qt, aW, cr, eZ, fe, lo, yq, mo, pL, iV, bz, pb, acp, adz, bTk, ac, axs, nB, bE, hc, ajd, cj, dy, fL, eo, y, Pr, hv); #c1(IRF6) c2(NP_001193625) c3(interferon regulatory factor 6 isoform 2) c4( ) c5(82374, 82375, 82376, 82377, 82378) c6( ) c7(FW, NT, n, aml, btF, Xt, eW, bTm, Na, amJ, aT, Nc, brT, bTl, aOX, bjy, aW, ff, Az, An, en, kk, bvU, atb, apM, bpv, Km, Vj, sH); #c1(IRF6) c2(NP_006138) c3(interferon regulatory factor 6 isoform 1) c4(162312, 162313, 162314) c5(162315, 162316, 162317, 162318, 162319, 162320, 162321, 162322, 162323, 162324, 162325, 162326, 162327, 162328) c6( ) c7(FW, NT, n, aml, btF, Xt, eW, bTm, Na, amJ, aT, Nc, brT, bTl, alX, bjy, aW, ff, Az, An, en, kk, bvU, atb, apM, bpv, Km, Vj, sH); #c1(IRF7) c2(NP_001563) c3(interferon regulatory factor 7 isoform a) c4(118987, 118988) c5(18989, 118990, 118991, 118992, 118993, 118994, 118995) c6( ) c7(gV, T, fR, al, jx, jh, aA, aj, mC, eA, F, ds, aT, h, DU, K, afu, Nr, A, aW, ff, Pg, eZ, aiT, lo, aF, po, en, as, t, bz, pb, Nq, dB, NL, bdP, aiM, fL, eg); #c1(IRF7) c2(NP_004020) c3(interferon regulatory factor 7 isoform b) c4(142894) c5(142895, 142896, 142897, 142898, 142899, 142900, 142901, 142902, 142903, 142904, 142905, 142906, 142907, 142908, 142909, 142910) c6( ) c7(gV, T, fR, al, jx, jh, aA, aj, mC, eA, F, ds, aT, h, DU, K, afu, Nr, A, aW, ff, Pg, eZ, aiT, lo, aF, po, en, as, t, bz, pb, Nq, dB, NL, bdP, aiM, fL, eg); #c1(IRF7) c2(NP_004022) c3(interferon regulatory factor 7 isoform d) c4(142911) c5(142912, 142913, 142914, 142915, 142916, 142917, 142918, 142919, 142920, 142921) c6( ) c7(gV, T, fR, al, jx, jh, aA, aj, mC, eA, F, ds, aT, h, DU, K, afu, Nr, A, aW, ff, Pg, eZ, aiT, lo, af, po, en, as, t, bz, pb, Nq, dB, NL, bdP, aiM, fL, eg); #c1(IRF8) c2(NP_002154) c3(interferon regulatory factor 8) c4(126000) c5(126001, 126002, 126003, 126004, 126005, 126006) c6( ) c7(cZ, T, iW, bTo, NT, aA, fd, jh, j, jE, ci, mF, bTn, V, bvE, q, F, aEX, aRD, aT, dz, hn, ey, K, apD, dd, an, jj, bc, pi, nP, A, iU, et, aW, Y, oV, eH, eZ, hQ, lo, af, hf, eF, iV, fq, pb, ck, iT, bE, cj, aS, eo, y, bt, kS); #c1(IRF9) c2(NP_006075) c3(interferon regulatory factor 9) c4(161687) c5(161688, 161689, 161690, 161691, 161692, 161693) c6( ) c7(gV, T, bM, Dw, id, jx, bB, fr, jE, EA, qN, V, eA, ad, F, ds, aCV, aT, h, Ds, DU, j, aVV, zy, ae, d, je, aW, Y, CT, aq, lo, iM, bE, KS, B); #c1(IRG1) c2(NP_001245335) c3(cis-aconitate decarboxylase) c4(93124, 93125) c5(93126, 93127, 93128, 93129, 93130, 93131, 93132, 93133, 93134, 93135, 93136, 93137) c6( ) c7(bL, T, dD, ZV, Zz, ea, e, aw, cz, sM, vr, bS, j, ct, eq, q, ad, av, ec, rS, aT, h, j, dQ, eN, hm, bf, cW, aW, Zt, r, Z, ac, nB, wE, aka, vb, hE, B); #c1(IRGM) c2(NP_001139277) c3(immunity-related GTPase family M protein) c4(59073) c5(59074, 59075, 59076, 59077, 59078, 59079) c6( ) c7(xN, T, dD, abV, iV, ig, oV, WP, ea, bTp, VT, aMA, n, aj, Ni, eq, rH, fe, aCK, y, eA); #c1(IRS1) c2(NP_005535) c3(insulin receptor substrate 1) c4(157227) c5(157228, 157229, 157230, 157231, 157232, 157233) c6( ) c7(T, aC, ea, e, fd, bB, ci, Hr, bv, aE, iv, K, dQ, hm, aW, fc, hl, g, eF, uC, aiv, zV, eM, bTq, m, dD, zX, NT, n, aj, ml, El, jy, dL, bl, uV, A, aq, bJ, Fi, JQ, mg, hl, at, B, bL, eR, oe, jx, cz, cR, v, j, yO, C, nS, pd, q, J, aT, bq, ae, af, bA, jf, kl, s, nO, Y, r, t, mf, sY, hl, aZ, Dd, dG, eq, aoz, ad, anv, eH, dq, bz, qa, bE, bC, ei, hE, xg, X); #c1(IRS2) c2(NP_003740) c3(insulin receptor substrate 2) c4(140545) c5(140546, 140547, 140548, 140549, 140550, 140551, 140552) c6( ) c7(bL, bM, T, dD, fM, aC, aZ, ea, oe, fd, NT, bs, aA, cz, aj, dG, j, v, Ea, bB, eq, pd, q, br, J, ad, dP, fU, eJ, Yn, aE, hW, bTr, aT, jy, dL, K, uV, bq, qM, rz, ae, af, bA, jf, kl, d, A, aW, ff, hl, r, fe, aq, en, g, t, bw, fq, bJ, bF, rR, bE, bo, X, mg, hl, mf, dy, m, cP, bC, ei, B); #c1(IRS4) c2(NP_003595) c3(insulin receptor substrate 4) c4(139454) c5(139455, 139456, 139457, 139458, 139459, 139460) c6( ) c7(bL, x, T, aC, iD, fR, af, Y, ff, hl, aj, CZ, K, uV, en, g, B, J); #c1(IRX1) c2(NP_077313) c3(iroquois-class homeodomain protein IRX-1) c4(215201) c5(215202, 215203, 215204, 215205, 215206, 215207, 215208) c6( ) c7(wu, oe, az, aw, Be, aA, cm, rO, ff, aF, wm, en, he, ik, aa); #c1(IRX2) c2(NP_150366) c3(iroquois-class homeodomain protein IRX-2) c4(228077) c5(228078, 228079, 228080, 228081, 228082, 228083, 228084, 228085, 228086, 228087, 228088) c6( ) c7(hP, Be, aa); #c1(IRX3) c2(NP_077312) c3(iroquois-class homeodomain protein IRX-3) c4(215193) c5(215194, 215195, 215196, 215197, 215198, 215199, 215200) c6( ) c7(bL, ac, v, aa, pN); #c1(IRX4) c2(NP_057442) c3(iroquois-class homeodomain protein IRX-4 isoform b) c4(190900) c5(190901, 190902, 190903, 190904, 190905, 190906, 190907, 190908, 190909, 190910, 190911, 190912, 190913, 190914, 190915) c6( ) c7(aC, cc, acl, e, oe, qK, M, vc, j, ae, af, ad); #c1(IRX5) c2(NP_001239126) c3(iroquois-class homeodomain protein IRX-5 isoform 2) c4( ) c5(88260, 88261, 88262, 88263, 88264) c6( ) c7(T, bjy, aNv, bE, TR, mG, bTt, Az, Be, cz, bB, bjB, bTs, aaw, Km, bD, cm, ae, ad, aa); #c1(IRX5) c2(NP_005844) c3(iroquois-class homeodomain protein IRX-5 isoform 1) c4(159821, 159822) c5(159823, 159824, 159825, 159826, 159827, 159828, 159829) c6( ) c7(T, bjy, aNv, bE, TR, mG, bTt, Az, Be, cz, bB, bjB, bTs, aaw, Km, bD, cm, ae, ad, aa); #c1(IRX5) c2(XP_011521111) c3(iroquois-class homeodomain protein IRX-5 isoform X1) c4( ) c5(316034, 316035, 316036) c6( ) c7(T, bjy, aNv, bE, TR, mG, bTt, Az, Be, cz, bB, bjB, bTs, aaw, Km, bD, cm, ae, ad, aa); #c1(ISCU) c2(NP_055116) c3(iron-sulfur cluster assembly enzyme ISCU, mitochondrial isoform ISCU1) c4(179038) c5(179039, 179040, 179041, 179042, 179043, 179044) c6( ) c7(fM, SE, kA, bTu, oj, jh, oh, aNG, hp, K, ci, TI, aPE, wT); #c1(ISCU) c2(NP_998760) c3(iron-sulfur cluster assembly enzyme ISCU, mitochondrial isoform ISCU2 precursor) c4(265868) c5(265869, 265870, 265871, 265872, 265873, 265874) c6( ) c7(fM, SE, kA, bTu, oj, jh, oh, aNG, hp, K, ci, TI, aPE, wT); #c1(ISCU) c2(XP_011536402) c3(iron-sulfur cluster assembly enzyme ISCU, mitochondrial isoform X2) c4(333707) c5(333708, 333709, 333710, 333711, 333712, 333713) c6( ) c7(fM, SE, kA, bTu, oj, jh, oh, aNG, hp, K, ci, Tl, aPE, wT); #c1(ISG20) c2(NP_001290163) c3(interferon-stimulated gene 20 kDa protein isoform a) c4(114994) c5(114995, 114996, 114997, 114998, 114999, 115000, 115001, 115002, 115003, 115004) c6( ) c7(bM, T, bO, CH, fd, cL, Vd, ci, bh, aY, aR, AW, xy, rw, dV, bRd, azm, rB, DY, bhm, bc, aa, li, JI, aW, az, kt, hN, eE, bw, mK, yd, ba, eM, Jr, iA, mx, cj, m, gn, abh, aO, aj, fr, cd, ai, Oe, xC, baA, ft, cc, kH, h, du, ox, Nr, A, zb, fe, po, bxh, jT, adz, Wt, yR, W, JM, bm, uz, aEG, auR, eo, fL, B, bL, fM, eR, fG, zN, OY, V, pd, eA, J, o, zn, eJ, lg, qp, aT, qG, bK, aFm, Cy, ae, hM, PK, bf, nO, iU, Y, bg, lo, t, f, iK, pb, ck, cT, sS, Ta, Dy, iM, ig, yW, fP, wR, Da, bxj, yH, byK, gT, bt, Kd, bjX, DA, jh, wV, jE, EA, hF, mC, ad, XB, LH, hX, wD, qh, hD, ji, bxf, iL, yF, uQ, d, je, cr, eZ, dN, Mz, hp, iV, bz, Nq, es, Bv, atM, Lj, z, XO, aEN, lf, ra, y, Tc); #c1(ISL1) c2(NP_002193) c3(insulin gene enhancer protein ISL-1) c4(126463, 126464) c5(126465, 126466, 126467, 126468, 126469, 126470, 126471, 126472, 126473, 126474, 126475, 126476, 126477) c6( ) c7(bL, cZ, mj, wK, aW, acl, yL, aw, oW, aT, nY, n, aj, v, j, qM, dd, bD, cm, cd, J); #c1(ISM1) c2(NP_543016) c3(isthmin-1 precursor) c4(233258, 233259) c5(233260, 233261, 233262, 233263, 233264, 233265, 233266, 233267, 233268, 233269, 233270, 233271, 233272, 233273, 233274, 233275) c6(233276, 233277, 233278, 233279) c7(T, aF, aw, an, aA); #c1(ISM2) c2(NP_872315) c3(isthmin-2 isoform 3 precursor) c4(256108) c5(256109, 256110, 256111, 256112, 256113) c6( ) c7(cc, ci, wK); #c1(ISM2) c2(NP_954993) c3(isthmin-2 isoform 1 precursor) c4(261521, 261522, 261523) c5(261524, 261525, 261526, 261527, 261528, 261529, 261530) c6( ) c7(cc, ci, wK); #c1(ISM2) c2(XP_011534791) c3(isthmin-2 isoform X1) c4(331233) c5(331234, 331235, 331236, 331237, 331238, 331239, 331240, 331241, 331242, 331243, 331244, 331245, 331246, 331247, 331248, 331249, 331250, 331251, 331252) c6( ) c7(cc, ci, wK); #c1(ISPD) c2(NP_001094896) c3(isoprenoid synthase domain-containing protein isoform a) c4(40779) c5(40780, 40781, 40782, 40783, 40784, 40785) c6( ) c7(ul, vJ, km, oj, UD, bTv, Cd); #c1(IST1) c2(NP_001257904) c3(IST1 homolog isoform a) c4( ) c5(95769, 95770, 95771, 95772, 95773, 95774, 95775, 95776, 95777, 95778) c6( ) c7(av, Bl, aW, ff, ht, aT, it, BF, en, hu, bz); #c1(IST1) c2(NP_001257905) c3(IST1 homolog isoform c) c4(95779, 95780, 95781) c5(95782, 95783, 95784, 95785, 95786, 95787, 95788, 95789) c6( ) c7(av, Bl, aW, ff, ht, aT, it, BF, en, hu, bz); #c1(IST1) c2(NP_001257906) c3(IST1 homolog isoform d) c4(95790) c5(95791, 95792, 95793, 95794, 95795, 95796) c6( ) c7(av, Bl, aW, ff, ht, aT, it, BF, en, hu, bz); #c1(IST1) c2(NP_055576) c3(IST1 homolog isoform b) c4(182038) c5(182039, 182040, 182041, 182042, 182043, 182044) c6( ) c7(av, Bl, aW, ff, ht, aT, it, BF, en, hu, bz); #c1(ISX) c2(NP_001290437) c3(intestine-specific homeobox) c4(115445) c5(115446, 115447, 115448, 115449, 115450, 115451, 115452, 115453, 115454, 115455, 115456) c6( ) c7(hH, B, aq); #c1(ISYNA1) c2(NP_001164409) c3(inositol-3-phosphate synthase 1 isoform 2) c4(66940, 66941) c5(66942, 66943, 66944, 66945, 66946, 66947) c6( ) c7(bL, bM, T, Dw, dA, jx, fN, aA, cz, bB, aj, wV, j, zh, C, qj, eh, q, J, cd, qK, dV, kH, cU, aT, K, JS, aDp, kl, lR, Jl, aW, Y, hl, cr, r, aiT, lo, aF, hp, g, iV, aaO, t, iK, pm, qa, ek, ck, es, iA, lp, bE, cj, aS, m, eg, y, ao, pC); #c1(ISYNA1) c2(NP_001240318) c3(inositol-3-phosphate synthase 1 isoform 4) c4( ) c5(88611, 88612, 88613, 88614, 88615, 88616, 88617, 88618, 88619, 88620, 88621, 88622, 88623) c6( ) c7(bL, bM, T, Dw, dA, jx, fN, aA, cz, bB, aj, wV, j, zh, C, qj, eh, q, J, cd, qK, dV, kH, cU, aT, K, JS, aDp, kl, lR, Jl, aW, Y, hl, cr, r, aiT, lo, af, hp, g, iV, aaO, t, iK, pm, qa, ek, ck, es, iA, lp, bE, cj, aS, m, eg, y, ao, pC); #c1(ISYNA1) c2(NP_057452) c3(inositol-3-phosphate synthase 1 isoform 1) c4(190953, 190954) c5(190955, 190956, 190957, 190958, 190959, 190960) c6( ) c7(bL, bM, T, Dw, dA, jx, fN, aA, cz, bB, aj, wV, j, zh, C, qj, eh, q, J, cd, qK, dV, kH, cU, aT, K, JS, aDp, kl, lR, Jl, aW, Y, hl, cr, r, aiT, lo, aF, hp, g, iV, aaO, t, iK, pm, qa, ek, ck, es, iA, lp, bE, cj, aS, m, eg, y, ao, pC); #c1(ITCH) c2(NP_001244066) c3(E3 ubiquitin-protein ligase ltchy homolog isoform 1) c4(91879) c5(91880, 91881, 91882, 91883, 91884, 91885) c6( ) c7(vU, hd, d, nM, nB, bTw, v, eo, ey, eE, y, V, atV); #c1(ITCH) c2(NP_001244067) c3(E3 ubiquitin-protein ligase ltchy homolog isoform 3) c4( ) c5(91886, 91887, 91888, 91889, 91890) c6( ) c7(vU, hd, d, nM, nB, bTw, v, eo, ey, eE, y, V, atV); #c1(ITCH) c2(NP_113671) c3(E3 ubiquitin-protein ligase ltchy homolog isoform 2) c4(222040, 222041) c5(222042, 222043, 222044, 222045, 222046, 222047, 222048) c6( ) c7(vU, hd, d, nM, nB, bTw, v, eo, ey, eE, y, V, atV); #c1(ITFG1) c2(NP_001291931) c3(T-cell immunomodulatory protein isoform 2) c4(116511) c5(116512, 116513, 116514, 116515, 116516) c6( ) c7(ey); #c1(ITFG1) c2(NP_110417) c3(T-cell immunomodulatory protein isoform 1 precursor) c4(220170, 220171, 220172) c5(220173, 220174, 220175, 220176, 220177, 220178, 220179, 220180, 220181, 220182, 220183, 220184, 220185, 220186, 220187, 220188, 220189, 220190, 220191) c6( ) c7(ey); #c1(ITFG3) c2(NP_114428) c3(protein 1 TFG3) c4(222707, 222708, 222709, 222710) c5(222711, 222712, 222713, 222714, 222715, 222716, 222717, 222718, 222719, 222720, 222721, 222722, 222723, 222724, 222725, 222726, 222727, 222728, 222729, 222730, 222731, 222732) c6( ) c7(awC); #c1(ITFG3) c2(XP_005255680) c3(protein ITFG3 isoform X3) c4(273785) c5(273786, 273787, 273788, 273789, 273790, 273791, 273792, 273793, 273794, 273795, 273796, 273797, 273798, 273799, 273800, 273801, 273802, 273803, 273804, 273805, 273806, 273807, 273808, 273809) c6( ) c7(awC); #c1(ITFG3) c2(XP_006721020) c3(protein ITFG3 isoform X2) c4(293805, 293806, 293807, 293808) c5(293809, 293810, 293811, 293812, 293813, 293814, 293815, 293816, 293817, 293818, 293819, 293820) c6( ) c7(awC); #c1(ITGA1) c2(NP_852478) c3(integrin alpha-1 precursor) c4(255019) c5(255020, 255021, 255022, 255023, 255024, 255025) c6( ) c7(bL, aC, wD, bTx, aCJ, yL, hl, ji, K, y, V, X, bA); #c1(ITGA10) c2(NP_001289969) c3(integrin alpha-10 isoform 2) c4(114872) c5(114873, 114874, 114875, 114876, 114877, 114878) c6( ) c7(wR, Y); #c1(ITGA10) c2(NP_001289970) c3(integrin alpha-10 isoform 3 precursor) c4(114879) c5(114880, 114881, 114882, 114883, 114884, 114885) c6( ) c7(wR, Y); #c1(ITGA10) c2(XP_011508383) c3(integrin alpha-10 isoform X3) c4(299069) c5(299070, 299071, 299072, 299073, 299074, 299075) c6( ) c7(wR, Y); #c1(ITGA1l) c2(NP_001004439) c3(integrin alpha-11 precursor) c4(20690, 20691, 20692) c5(20693, 20694, 20695, 20696, 20697, 20698, 20699, 20700, 20701, 20702, 20703, 20704, 20705, 20706, 20707, 20708, 20709, 20710, 20711, 20712, 20713, 20714, 20715, 20716) c6( ) c7(ba, eM, ff, bg, EQ, j, en, iu); #c1(ITGA11) c2(XP_005254285) c3(integrin alpha-Il isoform X2) c4(273140) c5( ) c6( ) c7(ba, eM, ff, bg, EQ, j, en, iu); #c1(ITGA2) c2(NP_002194) c3(integrin alpha-2 precursor) c4(126478, 126479, 126480, 126481) c5(126482, 126483, 126484, 126485, 126486, 126487, 126488, 126489, 126490, 126491, 126492, 126493, 126494, 126495, 126496, 126497, 126498, 126499, 126500, 126501, 126502, 126503, 126504, 126505) c6( ) c7(bL, T, dD, aC, eR, qb, bgP, ea, e, aw, wL, cz, ez, n, bLo, j, zh, ci, eq, q, ad, ai, TB, dP, bv, av, vC, ql, MP, M, aT, DU, K, ey, aWf, hH, adx, ae, af, qk, hm, eu, jf, eG, A, iU, cW, aW, Y, eZ, r, bEG, g, pL, cm, cM, sE, bz, uC, pb, LB, kT, ac, c, hc, vb, vy, qq, aS, Hb, X, us); #c1(ITGA2B) c2(NP_000410) c3(integrin alpha-IIb preproprotein) c4(8647) c5(8648, 8649, 8650, 8651, 8652, 8653) c6( ) c7(dD, eR, bTy, qb, ea, aw, oA, cz, bO, ez, QL, j, eq, fr, bED, hF, Fc, eA, vV, vC, bpp, rS, M, aT, LA, lG, cy, dz, bk, DU, jo, iY, cb, DY, af, eu, d, arl, cW, aW, Y, eZ, kD, bEG, bTz, pm, cm, cM, sE, uC, bw, ck, KT, ac, cA, pc, aka, vb, vy, aic, dW, dX, pw, jQ, y, jd); #c1(ITGA2B) c2(XP_011523051) c3(integrin alpha-IIb isoform X1) c4(317543, 317544) c5(317545, 317546, 317547, 317548, 317549, 317550) c6( ) c7(dD, eR, bTy, qb, ea, aw, oA, cz, bO, ez, QL, j, eq, fr, bED, hF, Fc, eA, vV, vC, bpp, rS, M, aT, LA, lG, cy, dz, bK, DU, jo, iY, cb, DY, af, eu, d, arl, cW, aW, Y, eZ, kD, bEG, bTz, pm, cm, cM, sE, uC, bw, ck, kT, ac, cA, pc, aka, vb, vy, aic, dW, dX, pw, jQ, y, jd); #c1(ITGA2B) c2(XP_011523052) c3(integrin alpha-IIb isoform X2) c4( ) c5(317551, 317552, 317553, 317554, 317555, 317556) c6( ) c7(dD, eR, bTy, qb, ea, aw, oA, cz, bO, ez, QL, j, eq, fr, bED, hF, Fc, eA, vV, vC, bpp, rS, M, aT, LA, lG, cy, dz, bK, DU, jo, iY, cb, DY, af, eu, d, arl, cW, aW, Y, eZ, kD, bEG, bTz, pm, cm, cM, sE, uC, bw, ck, kT, ac, cA, pc, aka, vb, vy, aic, dW, dX, pw, jQ, y, jd); #c1(ITGA3) c2(NP_002195) c3(integrin alpha-3 isoform a precursor) c4(126506, 126507, 126508, 126509) c5(126510, 126511, 126512, 126513, 126514, 126515, 126516, 126517, 126518, 126519, 126520, 126521, 126522, 126523, 126524, 126525, 126526, 126527, 126528, 126529, 126530, 126531, 126532, 126533, 126534, 126535) c6( ) c7(Gl, aw, ht, aA, bB, hy, ah, ci, C, j, BB, eA, ip, aT, vw, Gw, dL, lQ, bq, aa, d, B, ff, Pg, aF, en, g, vW, kP, bE, hc, cj, bTA, XN, Gu, X); #c1(ITGA4) c2(NP_000876) c3(integrin alpha-4 precursor) c4(16749, 16750) c5(16751, 16752, 16753, 16754, 16755, 16756, 16757, 16758, 16759, 16760, 16761, 16762) c6( ) c7(bM, aDT, jh, fd, ci, uE, mF, V, Qk, aT, h, ey, K, Lu, s, iU, qt, aW, Y, bvJ, Of, ff, Og, hp, en, g, eF, t, aZz, bN, ck, vy, cj, wR, ME, m, fD, y, bt, X); #c1(ITGA5) c2(NP_002196) c3(integrin alpha-5 precursor) c4(126536) c5(126537, 126538, 126539, 126540, 126541, 126542) c6( ) c7(T, id, fR, mr, qy, aA, bB, fd, lR, ci, fr, q, ad, MK, hX, dP, eA, av, Tg, aE, yt, aT, lG, By, ae, bc, DA, eG, d, B, aW, Y, Tc, ff, r, nY, uY, aF, CQ, en, hf, t, bz, pb, aZz, acW, eM, dB, bE, m, fD, X, vi); #c1(ITGA6) c2(NP_000201) c3(integrin alpha-6 isoform b precursor) c4(4466, 4467, 4468) c5(4469, 4470, 4471, 4472, 4473, 4474, 4475, 4476, 4477, 4478, 4479, 4480, 4481, 4482, 4483, 4484, 4485, 4486, 4487) c6( ) c7(T, dD, aC, fR, XJ, n, eq, ci, XM, eA, ad, aT, lQ, arL, d, aW, Y, eH, hl, uY, g, eF, t, bz, Zj, bN, bdQ, ck, kP, ac, XG, bt, it); #c1(ITGA6) c2(NP_001073286) c3(integrin alpha-6 isoform a precursor) c4(35750, 35751, 35752) c5(35753, 35754, 35755, 35756, 35757, 35758, 35759, 35760, 35761, 35762, 35763, 35764, 35765, 35766, 35767, 35768, 35769, 35770, 35771, 35772, 35773, 35774) c6( ) c7(T, dD, aC, fR, XJ, n, eq, ci, XM, eA, ad, aT, lQ, arL, d, aW, Y, eH, hl, uY, g, eF, t, bz, Zj, bN, bdQ, ck, kP, ac, XG, bt, it); #c1(ITGA7) c2(NP_001138468) c3(integrin alpha-7 isoform 1 precursor) c4(56806) c5(56807, 56808, 56809, 56810, 56811, 56812) c6( ) c7(cc, d, hW, ea, xs, Ad, oj, aq, bTB, X, ad); #c1(ITGA7) c2(NP_001138469) c3(integrin alpha-7 isoform 3) c4(56813) c5(56814, 56815, 56816, 56817, 56818, 56819) c6( ) c7(cc, d, hW, ea, xs, Ad, oj, aq, bTB, X, ad); #c1(ITGA7) c2(NP_002197) c3(integrin alpha-7 isoform 2 precursor) c4(126543) c5(126544, 126545, 126546, 126547, 126548, 126549) c6( ) c7(cc, d, hW, ea, xs, Ad, oj, aq, bTB, X, ad); #c1(ITGA7) c2(XP_005268896) c3(integrin alpha-7 isoform X1) c4(282162) c5(282163, 282164, 282165, 282166, 282167, 282168) c6( ) c7(cc, d, hW, ea, xs, Ad, oj, aq, bTB, X, ad); #c1(ITGA7) c2(XP_005268898) c3(integrin alpha-7 isoform X3) c4(282169, 282170, 282171) c5(282172, 282173, 282174, 282175, 282176, 282177, 282178, 282179, 282180, 282181, 282182, 282183, 282184, 282185, 282186, 282187, 282188, 282189) c6( ) c7(cc, d, hW, ea, xs, Ad, oj, aq, bTB, X, ad); #c1(ITGA7) c2(XP_011536589) c3(integrin alpha-7 isoform X9) c4(334085) c5(334086, 334087, 334088, 334089, 334090, 334091) c6( ) c7(cc, d, hW, ea, xs, Ad, oj, aq, bTB, X, ad); #c1(ITGA8) c2(NP_001278423) c3(integrin alpha-8 Isoform 2 preproprotein) c4(109742) c5(109743, 109744, 109745, 109746, 109747, 109748) c6( ) c7(sN, Y, aA, fd, ci, an, t, btr); #c1(ITGA8) c2(NP_003629) c3(integrin alpha-8 isoform 1 preproprotein) c4(139709) c5(139710, 139711, 139712, 139713, 139714, 139715) c6( ) c7(sN, Y, aA, fd, ci, an, t, btr); #c1(ITGA9) c2(NP_002198) c3(integrin alpha-9 precursor) c4(126550) c5(126551, 126552, 126553, 126554, 126555, 126556) c6( ) c7(jf, mU, T, av, py, BO, mW, mZ, aT, cz, mS, mY, bK, ci, mV, ik, bz, mT, mX); #c1(ITGAD) c2(NP_005344) c3(integrin alpha-D precursor) c4(155261, 155262) c5(155263, 155264, 155265, 155266, 155267, 155268, 155269, 155270, 155271, 155272, 155273) c6( ) c7(v, aky); #c1(ITGAD) c2(XP_011544144) c3(integrin alpha-D isoform X8) c4( ) c5(343307) c6( ) c7(v, aky); #c1(ITGAD) c2(XP_011544149) c3(integrin alpha-D isoform X13) c4(343308) c5( ) c6( ) c7(v, aky); #c1(ITGAD) c2(XP_011544150) c3(integrin alpha-D isoform X14) c4( ) c5(343309, 343310, 343311, 343312, 343313) c6( ) c7(v, aky); #c1(ITGAE) c2(NP_002199) c3(integrin alpha-E precursor) c4(126557) c5(126558, 126559, 126560, 126561, 126562) c6( ) c7(T, e, aw, j, av, mP, ds, bW, aAh, cU, hD, h, vB, eu, uQ, aa, Y, Of, Og, lo, Cv, ac, dB, cj); #c1(ITGAE) c2(XP_011522125) c3(integrin alpha-E isoform X1) c4( ) c5(316759) c6( ) c7(T, e, aw, j, av, mP, ds, bW, aAh, cU, hD, h, vB, eu, uQ, aa, Y, Of, Og, lo, Cv, ac, dB, cj); #c1(ITGAE) c2(XP_011522126) c3(integrin alpha-E isoform X2) c4( ) c5(316760) c6( ) c7(T, e, aw, j, av, mP, ds, bW, aAh, cU, hD, h, vB, eu, uQ, aa, Y, Of, Og, lo, Cv, ac, dB, cj); #c1(ITGAL) c2(NP_001107852) c3(integrin alpha-L isoform b precursor) c4(43025, 43026) c5(43027, 43028, 43029, 43030, 43031, 43032) c6( ) c7(oO, bM, T, aYZ, qb, iW, jx, bfM, fd, jh, wV, bS, ct, uE, V, mC, ai, Tz, eW, pe, tT, ayQ, dV, kH, aT, h, qG, dL, K, jj, an, hH, pk, ass, sX, dl, bc, eN, pi, je, aiV, cH, aW, Y, oV, axW, cr, awv, eZ, fe, beP, iV, Z, f, arJ, pb, ek, ck, iM, ac, iQ, hA, iT, cj, at, fL, y, bt); #c1(ITGAL) c2(NP_002200) c3(integrin alpha-L isoform a precursor) c4(126563, 126564) c5(126565, 126566, 126567, 126568, 126569, 126570, 126571) c6( ) c7(oO, bM, T, aYZ, qb, iW, jx, bfM, fd, jh, wV, bS, ct, uE, V, mC, ai, Tz, eW, pe, tT, ayQ, dV, kH, aT, h, qG, dL, K, jj, an, hH, pk, ass, sX, dl, bc, eN, pi, je, aiV, cH, aW, Y, oV, axW, cr, awv, eZ, fe, beP, iV, Z, f, arJ, pb, ek, ck, iM, ac, iQ, hA, iT, cj, at, fL, y, bt); #c1(ITGAL) c2(XP_005255370) c3(integrin alpha-L isoform X1) c4( ) c5(273626, 273627, 273628, 273629, 273630) c6( ) c7(oO, bM, T, aYZ, qb, iW, jx, bfM, fd, jh, wV, bS, ct, uE, V, mC, ai, Tz, eW, pe, tT, ayQ, dV, kH, aT, h, qG, dL, K, jj, an, hH, pk, ass, sX, dl, bc, eN, pi, je, aiV, cH, aW, Y, oV, axW, cr, awv, eZ, fe, beP, iV, Z, f, arJ, pb, ek, ck, iM, ac, iQ, hA, iT, cj, at, fL, y, bt); #c1(ITGAL) c2(XP_011544151) c3(integrin alpha-L isoform X3) c4( ) c5(343314, 343315, 343316, 343317, 343318, 343319) c6( ) c7(oO, bM, T, aYZ, qb, iW, jx, bfM, fd, jh, wV, bS, ct, uE, V, mC, ai, Tz, eW, pe, tT, ayQ, dV, kH, aT, h, qG, dL, K, jj, an, hH, pk, ass, sX, dl, bc, eN, pi, je, aiV, cH, aW, Y, oV, axW, cr, awv, eZ, fe, beP, iV, Z, f, arJ, pb, ek, ck, iM, ac, iQ, hA, iT, cj, at, fL, y, bt); #c1(ITGAM) c2(NP_000623) c3(integrin alpha-M isoform 2 precursor) c4(14028) c5(14029, 14030, 14031, 14032, 14033, 14034) c6( ) c7(oO, bM, T, ea, aFV, Tz, x, lE, K, H, hm, aW, zC, eE, cm, eM, iT, cj, hh, Ht, dD, aYZ, aw, oY, aj, uE, Tl, ai, eW, cU, h, dL, ey, A, PX, fJ, Og, kp, jT, ar, kT, W, at, uz, fL, Pr, bL, dA, fF, cR, j, C, V, q, J, ld, aT, qG, bK, jj, ae, af, dl, il, xR, Y, r, lo, en, arJ, pb, Cv, ck, hA, AH, hr, wR, dy, bt, jh, wV, eq, mC, ad, fX, tT, hD, ji, uX, ff, Of, cr, eZ, bz, ok, pc, z, aCl, MJ, gy, y); #c1(ITGAM) c2(NP_001139280) c3(integrin alpha-M isoform 1 precursor) c4(59080) c5(59081, 59082, 59083, 59084, 59085, 59086) c6( ) c7(oO, bM, T, ea, aFV, Tz, x, lE, K, H, hm, aW, zC, eE, cm, eM, iT, cj, hh, Ht, dD, aYZ, aw, oY, aj, uE, Tl, ai, eW, cU, h, dL, ey, A, PX, fJ, Og, kp, jT, ar, kT, W, at, uz, fL, Pr, bL, dA, fF, cR, j, C, V, q, J, ld, aT, qG, bK, jj, ae, af, dl, iU, xR, Y, r, lo, en, arJ, pb, Cv, ck, hA, AH, hr, wR, dy, bt, jh, wV, eq, mC, ad, fX, tT, hD, ji, uX, ff, Of, cr, eZ, bz, ok, pc, z, aCl, MJ, gy, y); #c1(ITGAV) c2(NP_001138471) c3(integrin alpha-V isoform 2 precursor) c4(56820) c5(56821, 56822, 56823, 56824, 56825, 56826) c6( ) c7(T, kh, dD, pf, ea, oe, qy, cz, fd, eq, jE, ci, lR, q, ad, El, hX, dP, eA, Tg, aE, afQ, aah, aT, cy, By, pj, ae, af, bc, hm, bA, eN, d, arl, s, aW, Y, Tc, ff, hl, r, nY, aF, CQ, en, g, eF, t, cm, bz, pb, adA, bN, acW, ac, eM, dB, bdP, m, fD, GF, y, rA, B, vi); #c1(ITGAV) c2(NP_001138472) c3(integrin alpha-V isoform 3 precursor) c4(56827) c5(56828, 56829, 56830, 56831, 56832, 56833) c6( ) c7(T, kh, dD, pf, ea, oe, qy, cz, fd, eq, jE, ci, lR, q, ad, El, hX, dP, eA, Tg, aE, afQ, aah, aT, cy, By, pj, ae, af, bc, hm, bA, eN, d, arl, s, aW, Y, Tc, ff, hl, r, nY, aF, CQ, en, g, eF, t, cm, bz, pb, adA, bN, acW, ac, eM, dB, bdP, m, fD, GF, y, rA, B, vi); #c1(ITGAV) c2(NP_002201) c3(integrin alpha-V isoform 1 precursor) c4(126572, 126573) c5(126574, 126575, 126576, 126577, 126578, 126579, 126580, 126581, 126582, 126583, 126584, 126585) c6( ) c7(T, kh, dD, pf, ea, oe, qy, cz, fd, eq, jE, ci, lR, q, ad, El, hX, dP, eA, Tg, aE, afQ, aah, aT, cy, By, pj, ae, af, bc, hm, bA, eN, d, arl, s, aW, Y, Tc, ff, hl, r, nY, aF, CQ, en, g, eF, t, cm, bz, pb, adA, bN, acW, ac, eM, dB, bdP, m, fD, GF, y, rA, B, vi); #c1(ITGAX) c2(NP_000878) c3(integrin alpha-X isoform 2 precursor) c4(16763) c5(16764, 16765, 16766, 16767, 16768, 16769) c6( ) c7(bM, T, dD, Dw, aZ, ea, l, fd, aj, v, eq, ci, V, eA, J, er, aoH, aao, zn, cc, aT, h, ey, acQ, jj, zy, H, af, dl, kl, asp, A, je, hG, Y, ff, cr, eZ, lo, en, iV, ho, bz, fq, Wt, ck, bax, cT, iM, AH, cj, wR, mf, eo, fD, ei, y, bt, aDi); #c1(ITGB1) c2(NP_391988) c3(integrin beta-1 isoform 1D precursor) c4(229031) c5(229032, 229033, 229034, 229035, 229036, 229037, 229038, 229039, 229040, 229041, 229042, 229043, 229044, 229045, 229046, 229047) c6(229048, 229049, 229050, 229051) c7(T, dD, ul, aC, aw, jh, aA, fd, n, aj, j, ci, C, eq, fr, BB, q, J, cd, ad, Jm, dP, eA, av, x, aE, cc, yt, aT, hD, vw, dL, bq, ae, af, eu, jf, d, B, aW, Y, ff, eH, eZ, r, aq, lo, kp, aF, en, g, nr, t, cM, bz, Z, op, eF, ik, ck, sS, BU, ac, eM, hc, vA, mx, hL, cj, QF, agf, dy, m, lS, oQ, XO, y, bt, X); #c1(ITGB1) c2(NP_596867) c3(integrin beta-1 isoform IA precursor) c4(234843, 234844, 234845, 234846, 234847, 234848, 234849, 234850, 234851, 234852) c5(234853, 234854, 234855, 234856, 234857, 234858, 234859, 234860, 234861, 234862, 234863, 234864, 234865, 234866, 234867, 234868, 234869, 234870, 234871, 234872, 234873, 234874, 234875, 234876, 234877, 234878, 234879, 234880, 234881, 234882, 234883, 234884, 234885, 234886, 234887, 234888, 234889, 234890, 234891, 234892, 234893, 234894, 234895, 234896, 234897, 234898, 234899, 234900, 234901, 234902, 234903, 234904, 234905, 234906, 234907, 234908, 234909, 234910, 234911, 234912, 234913, 234914, 234915, 234916, 234917, 234918, 234919, 234920, 234921, 234922, 234923, 234924, 234925, 234926, 234927, 234928) c6(234929, 234930, 234931, 234932, 234933, 234934, 234935, 234936, 234937, 234938, 234939, 234940, 234941, 234942, 234943, 234944, 234945, 234946) c7(T, dD, ul, aC, aw, jh, aA, fd, n, aj, j, ci, C, eq, fr, BB, q, J, cd, ad, Jm, dP, eA, av, x, aE, cc, yt, aT, hD, vw, dL, bq, ae, af, eu, jf, d, B, aW, Y, ff, eH, eZ, r, aq, lo, kp, aF, en, g, nr, t, cM, bz, Z, op, eF, iK, ck, sS, BU, ac, eM, hc, vA, mx, hL, cj, QF, agf, dy, m, lS, oQ, XO, y, bt, X); #c1(ITGB1BP1) c2(NP_004754) c3(integrin beta-1-binding protein 1 isoform 1) c4(149324, 149325, 149326) c5(149327, 149328, 149329, 149330, 149331, 149332, 149333, 149334, 149335, 149336, 149337, 149338, 149339, 149340, 149341, 149342) c6( ) c7(Az); #c1(ITGB1BP1) c2(NP_071729) c3(integrin beta-1-binding protein 1 isoform 2) c4(211804, 211805, 211806, 211807, 211808) c5(211809, 211810, 211811, 211812, 211813, 211814, 211815, 211816, 211817, 211818, 211819, 211820, 211821, 211822, 211823, 211824, 211825, 211826, 211827, 211828, 211829, 211830, 211831, 211832, 211833, 211834, 211835, 211836, 211837, 211838, 211839, 211840, 211841, 211842, 211843, 211844) c6( ) c7(Az); #c1(ITGB2) c2(NP_000202) c3(integrin beta-2 isoform 1 precursor) c4(4488, 4489, 4490, 4491, 4492, 4493, 4494, 4495) c5(4496, 4497, 4498, 4499, 4500, 4501, 4502, 4503, 4504, 4505, 4506, 4507, 4508, 4509, 4510, 4511, 4512, 4513, 4514, 4515, 4516, 4517, 4518, 4519, 4520, 4521, 4522, 4523, 4524, 4525, 4526, 4527, 4528, 4529, 4530, 4531, 4532, 4533, 4534, 4535, 4536, 4537, 4538, 4539, 4540, 4541, 4542, 4543, 4544, 4545, 4546, 4547, 4548, 4549, 4550, 4551, 4552, 4553, 4554) c6( ) c7(oO, bM, T, aC, fR, byb, ea, e, fd, aoj, aFV, Tz, bTC, aE, dv, dV, Ds, K, hH, pk, ass, hm, aiV, up, aW, mQ, awv, pL, iT, Jr, vy, cj, Nl, lS, dD, aYZ, bfM, n, BH, aj, ct, nA, uE, bfO, ai, eW, pe, kH, h, lG, ey, A, aCq, Cw, beP, cM, adz, ar, kT, W, at, va, AN, aHl, bL, Dw, dA, eR, aA, cR, fF, v, C, V, q, J, eA, zn, cF, fh, aT, cy, jj, an, sX, eu, arl, s, iU, xR, oV, Y, fO, r, lo, ahl, f, arJ, pb, ck, iM, ac, iQ, wR, fD, bt, zA, qb, dM, df, iW, jh, bS, eq, tT, hD, lQ, d, je, cH, cr, dN, aF, iV, Z, dr, aYt, ok, Km, aS, ei, y, agT, hv); #c1(ITGB2) c2(NP_001290167) c3(integrin beta-2 isoform 2) c4( ) c5(115005, 115006, 115007, 115008, 115009) c6( ) c7(oO, bM, T, aC, fR, byb, ea, e, fd, aoj, aFV, Tz, bTC, aE, dv, dV, Ds, K, hH, pk, ass, hm, aiV, up, aW, mQ, awv, pL, iT, Jr, vy, cj, Nl, lS, dD, aYZ, bfM, n, BH, aj, ct, nA, uE, bfO, ai, eW, pe, kH, h, lG, ey, A, aCq, Cw, beP, cM, adz, ar, kT, W, at, va, AN, aHl, bL, Dw, dA, eR, aA, cR, fF, v, C, V, q, J, eA, zn, cF, fh, aT, cy, jj, an, sX, eu, arl, s, iU, xR, oV, Y, fO, r, lo, ahl, f, arJ, pb, ck, iM, ac, iQ, wR, fD, bt, zA, qb, dM, df, iW, jh, bS, eq, tT, hD, lQ, d, je, cH, cr, dN, aF, iV, Z, dr, aYt, ok, Km, aS, ei, y, agT, hv); #c1(ITGB3) c2(NP_000203) c3(integrin beta-3 precursor) c4(4555, 4556) c5(4557, 4558, 4559, 4560, 4561, 4562, 4563, 4564, 4565, 4566, 4567, 4568, 4569, 4570, 4571, 4572) c6( ) c7(bM, T, ea, e, fd, lR, ci, dY, vV, vh, M, JT, GO, dQ, hH, DY, eN, hm, aW, mQ, vs, aB, g, eF, bw, uC, y, fT, eM, wE, bqd, sU, dW, m, pw, P, dD, xu, sQ, bTy, aw, ez, QL, aGU, zP, bTG, br, TB, ec, vC, ql, h, lG, nm, eV, DU, ux, he, bsk, MQ, fe, yq, cM, sE, KT, W, vb, qq, te, eo, Pr, abW, bL, wj, dA, eR, mr, wL, oe, aGu, jx, ex, cz, vv, v, j, bX, Wa, q, eA, rS, aT, Nc, bq, rz, qk, eu, kl, arl, iU, Y, fO, r, aDm, bEG, en, t, pb, el, bN, ck, cT, sn, c, ac, hc, mG, tz, dy, bTE, sY, kh, uq, qb, iW, vr, oR, bLo, uu, eq, atK, ad, XB, tX, dP, av, jo, adx, sj, VX, cW, aaB, ff, eZ, kD, bpA, BG, bz, bTD, es, bTF, pc, pE, ag, gZ, aS, XO, zk, gq, us); #c1(ITGB4) c2(NP_000204) c3(integrin beta-4 isoform 1 precursor) c4( ) c5(4573, 4574, 4575, 4576, 4577) c6( ) c7(T, dD, ea, e, qx, eq, ci, aRE, XM, ad, av, Db, ju, qK, aT, hP, lQ, arL, arO, d, bTH, aW, ff, en, g, as, bw, dr, ck, nB, z, cj, XG, XO, XN, it, X); #c1(ITGB4) c2(NP_001005619) c3(integrin beta-4 isoform 2 precursor) c4( ) c5(21986, 21987, 21988, 21989, 21990, 21991, 21992, 21993) c6( ) c7(T, dD, ea, e, qx, eq, ci, aRE, XM, ad, av, Db, ju, qK, aT, hP, lQ, arL, arO, d, bTH, aW, ff, en, g, as, bw, dr, ck, nB, z, cj, XG, XO, XN, it, X); #c1(ITGB4) c2(NP_001005731) c3(integrin beta-4 isoform 3 precursor) c4( ) c5(21994, 21995, 21996, 21997, 21998, 21999) c6( ) c7(T, dD, ea, e, qx, eq, ci, aRE, XM, ad, av, Db, ju, qK, aT, hP, lQ, arL, arO, d, bTH, aW, ff, en, g, as, bw, dr, ck, nB, z, cj, XG, XO, XN, it, X); #c1(ITGB4) c2(XP_006721930) c3(integrin beta-4 isoform X4) c4(294402) c5(294403) c6( ) c7(T, dD, ea, e, qx, eq, ci, aRE, XM, ad, av, Db, ju, qK, aT, hP, lQ, arL, arO, d, bTH, aW, ff, en, g, as, bw, dr, ck, nB, z, cj, XG, XO, XN, it, X); #c1(ITGB4) c2(XP_006721931) c3(integrin beta-4 isoform X5) c4(294404) c5(294405, 294406) c6( ) c7(T, dD, ea, e, qx, eq, ci, aRE, XM, ad, av, Db, ju, qK, aT, hP, lQ, arL, arO, d, bTH, aW, ff, en, g, as, bw, dr, ck, nB, z, cj, XG, XO, XN, it, X); #c1(ITGB4) c2(XP_006721933) c3(integrin beta-4 isoform X7) c4(294407) c5( ) c6( ) c7(T, dD, ea, e, qx, eq, ci, aRE, XM, ad, av, Db, ju, qK, aT, hP, lQ, arL, arO, d, bTH, aW, ff, en, g, as, bw, dr, ck, nB, z, cj, XG, XO, XN, it, X); #c1(ITGB5) c2(NP_002204) c3(integrin beta-5 precursor) c4(126586) c5(126587, 126588, 126589, 126590, 126591, 126592) c6( ) c7(Yn, dD, aW, aA, aT, hz, at, af, eq, ci, h, pj); #c1(ITGB6) c2(NP_000879) c3(integrin beta-6 isoform a precursor) c4(16770) c5(16771, 16772, 16773, 16774, 16775, 16776, 16777, 16778, 16779, 16780) c6( ) c7(bL, F, ac, aW, oe, aCx, hl, uR, aT, fQ, ff, eA, en, Ud, alV, X, eu, asw); #c1(ITGB7) c2(NP_000880) c3(integrin beta-7 precursor) c4(16781, 16782) c5(16783, 16784, 16785, 16786, 16787, 16788, 16789, 16790, 16791, 16792, 16793, 16794, 16795, 16796, 16797, 16798) c6( ) c7(Nq, bM, Ht, Nr, iM, je, jh, kH, h, po, fe); #c1(ITGB8) c2(NP_002205) c3(integrin beta-8 precursor) c4(126593) c5(126594, 126595, 126596, 126597, 126598, 126599, 126600, 126601, 126602, 126603, (26604) c6( ) c7(LB, eM, hP, dP, pb); #c1(ITGB8) c2(XP_011513695) c3(integrin beta-8 isoform X2) c4( ) c5(305994) c6( ) c7(LB, eM, hP, dP, pb); #c1(ITGB8) c2(XP_011513696) c3(integrin beta-8 isoform X3) c4(305995) c5( ) c6( ) c7(LB, eM, hP, dP, pb); #c1(ITGBL1) c2(NP_001258683) c3(integrin beta-like protein 1 isoform 4) c4(96328) c5(96329, 96330, 96331, 96332, 96333, 96334, 96335, 96336) c6( ) c7(bX); #c1(ITGBL1) c2(NP_001258685) c3(integrin beta-like protein 1 isoform 3) c4(96337) c5(96338, 96339, 96340, 96341, 96342, 96343, 96344, 96345, 96346, 96347, 96348, 96349) c6( ) c7(bX); #c1(ITGBL1) c2(NP_004782) c3(integrin beta-like protein 1 isoform 1 precursor) c4(149583, 149584) c5(149585, 149586, 149587, 149588, 149589, 149590, 149591, 149592) c6( ) c7(bX); #c1(ITIH1) c2(NP_002206) c3(inter-alpha-trypsin inhibitor heavy chain H1 isoform a precursor) c4(126605, 126606, 126607, 126608) c5(126609, 126610, 126611, 126612, 126613, 126614, 126615, 126616, 126617, 126618, 126619, 126620, 126621, 126622, 126623, 126624, 126625, 126626, 126627, 126628, 126629, 126630, 126631, 126632) c6( ) c7(T, bO, aT, aq, bK, ci, bz); #c1(ITIH2) c2(NP_002207) c3(inter-alpha-trypsin inhibitor heavy chain H2 precursor) c4(126633) c5(126634, 126635, 126636, 126637, 126638, 126639) c6( ) c7(aW, t, aT); #c1(ITIH3) c2(NP_002208) c3(inter-alpha-trypsin inhibitor heavy chain H3 preproprotein) c4(126640, 126641, 126642) c5(126643, 126644, 126645, 126646, 126647, 126648, 126649, 126650, 126651, 126652, 126653, 126654, 126655, 126656, 126657, 126658, 126659, 126660, 126661, 126662, 126663, 126664, 126665, 126666) c6( ) c7(aw, gl, bS, bK, ci, he, B); #c1(ITIH3) c2(XP_006713193) c3(inter-alpha-trypsin inhibitor heavy chain H3 isoform X3) c4(287058) c5( ) c6( ) c7(aw, gl, bS, bK, ci, he, B); #c1(ITIH4) c2(NP_001159921) c3(inter-alpha-trypsin inhibitor heavy chain H4 isoform 2 precursor) c4(64965, 64966, 64967) c5(64968, 64969, 64970, 64971, 64972, 64973, 64974, 64975, 64976, 64977, 64978, 64979, 64980, 64981, 64982, 64983, 64984, 64985) c6( ) c7(DS, oO, Ke, gA, ze, EA, eW, hb, aT, h, bK, K, zy, CB, afu, lx, cH, aW, aCH, lo, aoD, beP, kk, aoG, as, cm, asE, ck, cl, iQ, NL, Pl, EY, hh); #c1(ITIH4) c2(NP_002209) c3(inter-alpha-trypsin inhibitor heavy chain H4 isoform 1 precursor) c4(126667, 126668) c5(126669, 126670, 126671, 126672, 126673, 126674, 126675, 126676, 126677, 126678, 126679, 126680) c6( ) c7(DS, oO, Ke, gA, ze, EA, eW, hb, aT, h, bK, K, zy, CB, afu, lx, cH, aW, aCH, lo, aoD, beP, kk, aoG, as, cm, asE, ck, cl, iQ, NL, Pl, EY, hh); #c1(ITIH5) c2(NP_001001851) c3(inter-alpha-trypsin inhibitor heavy chain H5 isoform 3 precursor) c4(19027, 19028) c5(19029, 19030, 19031, 19032, 19033, 19034, 19035, 19036, 19037, 19038, 19039, 19040) c6( ) c7(nB, qt, aW, eL, aw, aT, n, dL, v, ci, AO, qk); #c1(ITIH5) c2(NP_085046) c3(inter-alpha-trypsin inhibitor heavy chain H5 isoform 1 precursor) c4(219193, 219194) c5(219195, 219196, 219197, 219198, 219199, 219200, 219201, 219202, 219203, 219204, 219205, 219206) c6( ) c7(nB, qt, aW, eL, aw, aT, n, dL, v, ci, AO, qk); #c1(ITIH5) c2(NP_116206) c3(inter-alpha-trypsin inhibitor heavy chain H5 isoform 2) c4(225752) c5(225753, 225754, 225755, 225756, 225757, 225758) c6( ) c7(nB, qt, aW, eL, aw, aT, n, dL, v, ci, AO, qk); #c1(ITK) c2(NP_005537) c3(tyrosine-protein kinase ITK/TSK) c4(157234) c5(157235, 157236, 157237, 157238, 157239, 157240) c6( ) c7(bM, T, aC, Mb, jh, aA, bB, n, wV, fr, ci, pz, AO, q, ad, eA, zn, iu, asG, hd, aE, apJ, aCV, aT, h, dL, ey, K, rE, on, ae, eL, na, uQ, bTJ, bTI, nO, Hl, aW, Y, oV, r, nY, aq, Qm, kp, aF, g, cl, t, fq, bz, LB, kP, eM, nB, bE, X, oh, np, Hb, m, adg, it, B, cn, qc); #c1(ITLN1) c2(NP_060095) c3(intelectin-1 precursor) c4(194099) c5(194100, 194101, 194102, 194103, 194104, 194105) c6( ) c7(bL, bM, T, kl, ib, aA, fe, aj, v, aF, ci, K); #c1(ITLN2) c2(XP_011507512) c3(intelectin-2 isoform X1) c4(297461) c5(297462, 297463, 297464, 297465, 297466, 297467) c6( ) c7(bL, aj); #c1(ITM2A) c2(NP_001165052) c3(integral membrane protein 2A isoform 2) c4(67262) c5(67263, 67264, 67265, 67266, 67267, 67268) c6( ) c7(hD, bX); #c1(ITM2A) c2(NP_004858) c3(integral membrane protein 2A isoform 1) c4(150371) c5(150372, 150373, 150374, 150375, 150376, 150377) c6( ) c7(hD, bX); #c1(ITM2B) c2(NP_068839) c3(integral membrane protein 2B) c4(210873) c5(210874, 210875, 210876, 210877, 210878, 210879) c6( ) c7(axS, cZ, tj, lR, mr, aW, bW, aal, nx, l, aT, U, cz, R, dd, H, on, af, acM, ad, L); #c1(ITM2C) c2(NP_001012532) c3(integral membrane protein 2C isoform 3) c4(25080) c5(25081, 25082, 25083, 25084, 25085, 25086) c6( ) c7(af, ac, bTK); #c1(ITM2C) c2(NP_001012534) c3(integral membrane protein 2C isoform 2) c4(25094) c5(25095, 25096, 25097, 25098, 25099, 25100) c6( ) c7(af, ac, bTK); #c1(ITM2C) c2(NP_001274170) c3(integral membrane protein 2C isoform 1) c4(106087, 106088) c5(106089, 106090, 106091, 106092, 106093, 106094, 106095, 106096, 106097, 106098, 106099, 106100) c6( ) c7(af, ac, bTK); #c1(ITPA) c2(NP_001254552) c3(inosine triphosphate pyrophosphatase isoform c) c4(94667) c5(94668, 94669, 94670, 94671, 94672, 94673) c6( ) c7(gV, bTL, al, jx, x, F, fS, oW, h, DU, hG, eZ, ge, fe, S, iV, gk, mO, hA, dc, k, y, ra, aeA); #c1(ITPA) c2(NP_258412) c3(inosine triphosphate pyrophosphatase isoform a) c4(228697) c5(228698, 228699, 228700, 228701, 228702, 228703) c6( ) c7(gV, bTL, al, jx, x, F, fS, oW, h, DU, hG, eZ, ge, fe, S, iV, gk, mO, hA, dc, k, y, ra, aeA); #c1(ITPA) c2(NP_852470) c3(inosine triphosphate pyrophosphatase isoform b) c4(255005) c5(255006, 255007, 255008, 255009, 255010, 255011) c6( ) c7(gV, bTL, al, jx, x, F, fS, oW, h, DU, hG, eZ, ge, fe, S, iV, gk, mO, hA, dc, k, y, ra, aeA); #c1(ITPK1) c2(NP_001136065) c3(inositol-tetrakisphosphate 1-kinase isoform a) c4(54188, 54189, 54190, 54191) c5(54192, 54193, 54194, 54195, 54196, 54197, 54198, 54199, 54200, 54201, 54202, 54203, 54204, 54205, 54206, 54207, 54208, 54209, 54210, 54211, 54212, 54213) c6( ) c7(lp, dy, ac); #c1(ITPK1) c2(NP_001136066) c3(inositol-tetrakisphosphate 1-kinase isoform b) c4(54214) c5(54215, 54216, 54217, 54218, 54219, 54220, 54221, 54222, 54223, 54224, 54225, 54226, 54227, 54228, 54229, 54230) c6( ) c7(lp, dy, ac); #c1(ITPKA) c2(NP_002211) c3(inositol-trisphosphate 3-kinase A) c4(126681) c5(126682, 126683, 126684, 126685, 126686, (26687) c6( ) c7(T, ff, en); #c1(ITPKC) c2(XP_006723467) c3(inositol-trisphosphate 3-kinase C isoform X1) c4(295214) c5(295215, 295216, 295217, 295218, 295219, 295220) c6( ) c7(od, if, lf, aS, qN, bTM, cn); #c1(ITPR1) c2(NP_001093422) c3(inositol 1,4,5-trisphosphate receptor type 1 isoform 1) c4(40265, 40266) c5(40267, 40268, 40269, 40270, 40271, 40272, 40273, 40274, 40275, 40276, 40277, 40278) c6( ) c7(bM, eX, fN, rf, uZ, cd, ad, lG, eV, dL, bk, K, ae, bmo, eL, xs, uY, EH, bTN, cm, cM, bTO, rR, ky, KB, U); #c1(ITPR1) c2(NP_001161744) c3(inositol 1,4,5-trisphosphate receptor type 1 isoform 3) c4(65902) c5(65903, 65904, 65905, 65906, 65907, 65908, 65909, 65910, 65911, 65912) c6( ) c7(bM, eX, fN, rf, uZ, cd, ad, lG, eV, dL, bK, K, ae, bmo, eL, xs, uY, EH, bTN, cm, cM, bTO, rR, ky, kB, U); #c1(ITPR1) c2(NP_002213) c3(inositol 1,4,5-trisphosphate receptor type 1 isoform 2) c4(126688) c5(126689, 126690, 126691, 126692, 126693, 126694, 126695, 126696, 126697, 126698, 126699, 126700) c6( ) c7(bM, eX, fN, rf, uZ, cd, ad, lG, eV, dL, bK, K, ae, bmo, eL, xs, uY, EH, bTN, cm, cM, bTO, rR, ky, kB, U); #c1(ITPR1) c2(XP_011531985) c3(inositol 1,4,5-trisphosphate receptor type 1 isoform X2) c4(328014, 328015, 328016) c5(328017, 328018, 328019, 328020, 328021, 328022, 328023, 328024, 328025, 328026, 328027, 328028, 328029, 328030, 328031, 328032, 328033) c6( ) c7(bM, eX, fN, rf, uZ, cd, ad, lG, eV, dL, bK, K, ae, bmo, eL, xs, uY, EH, bTN, cm, cM, bTO, rR, ky, kB, U); #c1(ITPR1) c2(XP_011531986) c3(inositol 1,4,5-trisphosphate receptor type 1 isoform X3) c4( ) c5(328034) c6( ) c7(bM, ex, fN, rf, uZ, cd, ad, lG, eV, dL, bK, K, ae, bmo, eL, xs, uY, EH, bTN, cm, cM, bTO, rR, ky, kB, U); #c1(ITPR1) c2(XP_011531992) c3(inositol 1,4,5-trisphosphate receptor type 1 isoform X12) c4(328035) c5( ) c6( ) c7(bM, eX, fN, rf, uZ, cd, ad, lG, eV, dL, bK, K, ae, bmo, eL, xs, uY, EH, bTN, cm, cM, bTO, rR, ky, kB, U); #c1(ITPR2) c2(NP_002214) c3(inositol 1,4,5-trisphosphate receptor type 2) c4(126701, (26702) c5(126703, 126704, 126705, 126706, 126707, 126708, 126709, 126710, 126711, 126712, 126713, 126714) c6( ) c7(c, oe, dL, bK, Vd, fr, cm, eL, bmo, y); #c1(ITPR3) c2(NP_002215) c3(inositol 1,4,5-trisphosphate receptor type 3) c4(126715) c5(126716, 126717, 126718, 126719, 126720, 126721) c6( ) c7(rR, A, kB, dH, xs, eZ, aA, hD, uY, dL, bk, K, v, cm, eL, cd, y); #c1(ITSN1) c2(NP_001001132) c3(intersectin-1 isoform ITSN-s) c4( ) c5(18135, 18136, 18137, 18138, 18139, 18140) c6( ) c7(cm, f, T, af, eA); #c1(ITSN1) c2(NP_003015) c3(intersectin-1 isoform ITSN-l) c4(134439, 134440, 134441, 134442) c5(134443, 134444, 134445, 134446, 134447, 134448, 134449, 134450, 134451, 134452, 134453, 134454, 134455, 134456, 134457, 134458, 134459, 134460, 134461, 134462, 134463, 134464, 134465, 134466) c6( ) c7(cm, f, T, af, eA); #c1(ITSN1) c2(XP_005261083) c3(intersectin-1 isoform X6) c4(277134, 277135, 277136, 277137, 277138) c5(277139, 277140, 277141, 277142, 277143, 277144, 277145, 277146, 277147, 277148, 277149, 277150, 277151, 277152, 277153, 277154, 277155, 277156, 277157, 277158, 277159, 277160, 277161, 277162, 277163, 277164, 277165, 277166, 277167, 277168, 277169, 277170, 277171, 277172, 277173, 277174, 277175, 277176, 277177, 277178, 277179) c6( ) c7(cm, f, T, af, eA); #c1(ITSN1) c2(XP_005261088) c3(intersectin-1 isoform X3) c4(277180, 277181, 277182) c5( ) c6( ) c7(cm, f, T, af, eA); #c1(ITSN1) c2(XP_011527993) c3(intersectin-1 isoform X2) c4(323052) c5( ) c6( ) c7(cm, f, T, af, BA); #c1(ITSN1) c2(XP_011527994) c3(intersectin-1 isoform X4) c4(323053) c5(323054, 323055, 323056, 323057, 323058, 323059, 323060, 323061, 323062, 323063, 323064, 323065) c6( ) c7(cm, f, T, af, eA); #c1(ITSN1) c2(XP_011527995) c3(intersectin-1 isoform X5) c4(323066, 323067, 323068) c5(323069, 323070, 323071, 323072, 323073, 323074, 323075, 323076, 323077, 323078, 323079, 323080, 323081, 323082, 323083, 323084, 323085, 323086, 323087) c6( ) c7(cm, f, T, af, eA); #c1(ITSN2) c2(NP_006268) c3(intersectin-2 isoform 1) c4(163443) c5(163444, 163445, 163446, 163447, 163448, 163449, 163450) c6( ) c7(fN, an); #c1(ITSN2) c2(NP_062541) c3(intersectin-2 isoform 3) c4(201889, 201890) c5(201891, 201892, 201893, 201894, 201895, 201896, 201897, 201898, 201899, 201900) c6( ) c7(fN, an); #c1(ITSN2) c2(NP_671494) c3(intersectin-2 isoform 2) c4(242039) c5(242040, 242041, 242042, 242043, 242044, 242045, 242046, 242047, 242048, 242049, 242050) c6( ) c7(fN, an); #c1(IVD) c2(NP_001152980) c3(isovaleryl-CoA dehydrogenase, mitochondrial isoform 2 precursor) c4(60292) c5(60293, 60294, 60295, 60296, 60297, 60298) c6( ) c7(h, auu, ac); #c1(IVD) c2(NP_002216) c3(isovaleryl-CoA dehydrogenase, mitochondrial isoform 1 precursor) c4(126722, 126723) c5(126724, 126725, 126726, 126727, 126728, 126729, 126730, 126731, 126732, 126733, 126734, 126735) c6( ) c7(h, auu, ac); #c1(IVL) c2(XP_006711363) c3(involucrin isoform X1) c4(285939, 285940) c5(285941, 285942, 285943, 285944, 285945, 285946, 285947, 285948, 285949, 285950, 285951, 285952, 285953, 285954, 285955, 285956) c6( ) c7(aAW, T, sS, Jr, Kv, hk, eE, ht, ff, cr, Nl, h, Kh, Xk, hu, en, qN, ik, akG, bTP); #c1(IVNS1ABP) c2(NP_006460) c3(influenza virus NS1A-binding protein) c4(165381, 165382) c5(165383, 165384, 165385, 165386, 165387, 165388, 165389, 165390, 165391, 165392, 165393, 165394, 165395) c6( ) c7(bL, Dw, NK, T, bpn, jx, aj, v, ai, J, ad, ds, cU, vd, atJ, hl, aGQ, lo, AH, Uq, aiM, rH, eQ, y, Ro); #c1(IVNS1ABP) c2(XP_005244900) c3(influenza virus NS1A-binding protein isoform X4) c4( ) c5(266156, 266157, 266158, 266159, 266160) c6( ) c7(bL, Dw, NK, T, bpn, jx, aj, v, ai, J, ad, ds, cU, vd, atJ, hl, aGQ, lo, AH, Uq, aiM, rH, eQ, y, Ro); #c1(IVNS1ABP) c2(XP_011507381) c3(influenza virus NS1A-binding protein isoform X1) c4(297242) c5( ) c6( ) c7(bL, Dw, NK, T, bpn, jx, aj, v, ai, J, ad, ds, cU, vd, atJ, hl, aGQ, lo, AH, Uq, aiM, rH, eQ, y, Ro); #c1(IVNS1ABP) c2(XP_011507382) c3(influenza virus NS1A-binding protein isoform X2) c4(297243) c5(297244, 297245, 297246, 297247, 297248, 297249) c6( ) c7(bL, Dw, NK, T, bpn, jx, aj, v, ai, J, ad, ds, cU, vd, atJ, hl, aGQ, lo, AH, Uq, aiM, rH, eQ, y, Ro); #c1(IYD) c2(NP_001158166) c3(iodotyrosine dehalogenase 1 isoform 1) c4(63239, 63240, 63241) c5(63242, 63243, 63244, 63245, 63246, 63247, 63248, 63249, 63250, 63251, 63252, 63253, 63254, 63255, 63256, 63257, 63258, 63259) c6( ) c7(qJ, Vp, hd, ac, nB, bzb, hD, ww, gT, aQy, bTQ); #c1(IYD) c2(NP_001158167) c3(iodotyrosine dehalogenase 1 isoform 3) c4(63260) c5(63261, 63262, 63263, 63264, 63265, 63266) c6( ) c7(qJ, Vp, hd, ac, nB, bzb, hD, ww, gT, ally, bTQ); #c1(IYD) c2(NP_981932) c3(iodotyrosine dehalogenase 1 isoform 2) c4(263170) c5(263171, 263172, 263173, 263174, 263175, 263176) c6( ) c7(qJ, Vp, hd, ac, nB, bzb, hD, ww, gT, aQy, bTQ); #c1(IZUMO1) c2(XP_005258850) c3(izumo sperm-egg fusion protein 1 isoform X2) c4( ) c5(275836) c6( ) c7(bTR, gU, ND); #c1(IZUMO1) c2(XP_011525115) c3(izumo sperm-egg fusion protein 1 isoform X1) c4(319536) c5(319537, 319538, 319539, 319540, 319541) c6( ) c7(bTR, gU, ND); #c1(JADE1) c2(NP_001274366) c3(protein Jade-1 isoform 3) c4(106285) c5(106286, 106287, 106288, 106289, 106290, 106291) c6( ) c7(es, iA, dE, go, dy, fr, VB); #c1(JADE1) c2(NP_001274368) c3(protein Jade-1 isoform 1) c4(106305, 106306, 106307, 106308, 106309, 106310, 106311) c5(106312, 106313, 106314, 106315, 106316, 106317, 106318, 106319, 106320, 106321, 106322, 106323, 106324, 106325, 106326, 106327, 106328, 106329, 106330, 106331, 106332) c6( ) c7(es, iA, dE, go, dy, fr, VB); #c1(JADE1) c2(NP_001274370) c3(protein Jade-1 isoform 2) c4(106333) c5(106334, 106335, 106336, 106337, 106338, 106339, 106340, 106341, 106342, 106343, 106344, 106345, 106346, 106347, 106348, 106349, 106350, 106351, 106352, 106353, 106354, 106355, 106356, 106357, 106358, 106359, 106360, 106361, 106362, 106363, 106364, 106365, 106366, 106367, 106368, 106369, 106370, 106371, 106372, 106373, 106374) c6( ) c7(es, iA, dE, go, dy, fr, VB); #c1(JADE1) c2(XP_011530562) c3(protein Jade-1 isoform X2) c4(326485) c5( ) c6( ) c7(es, iA, dE, go, dy, fr, VB); #c1(JADE2) c2(NP_001276914) c3(protein Jade-2 isoform 2) c4(108610, 108611) c5(108612, 108613, 108614, 108615, 108616, 108617) c6( ) c7(v, cd); #c1(JADE2) c2(XP_011541595) c3(protein Jade-2 isoform X2) c4(340133) c5(340134, 340135, 340136, 340137, 340138, 340139) c6( ) c7(v, cd); #c1(JADE2) c2(XP_011541596) c3(protein Jade-2 isoform X1) c4(340140) c5(340141, 340142, 340143, 340144, 340145, 340146, 340147, 340148, 340149, 340150, 340151, 340152, 340153, 340154, 340155, 340156, 340157, 340158) c6( ) c7(v, cd); #c1(JAG1) c2(NP_000205) c3(protein jagged-1 precursor) c4(4578, 4579) c5(4580, 4581, 4582, 4583, 4584, 4585, 4586, 4587, 4588, 4589, 4590, 4591) c6( ) c7(T, aC, al, fd, ci, bTU, Hr, ip, aE, anl, iv, Qe, ajT, Jl, aW, az, hl, Au, g, hJ, cm, bw, zV, iT, acw, cj, m, bga, n, aj, aLl, md, afo, zw, bTS, Jn, asT, bmF, Cw, blP, ack, yq, cM, bD, B, jx, aA, j, V, q, J, er, eA, Mg, qK, aT, aaw, jj, ae, af, bA, Y, r, lo, t, iK, ck, bLr, beH, beJ, gX, dy, bt, hS, bTT, rl, jh, ad, hX, Jm, av, py, MS, gj, d, rM, aF, Fq, bz, dr, LB, gk, adh, lM, y, kS, Ex, BA); #c1(JAG2) c2(NP_002217) c3(protein jagged-2 isoform a precursor) c4(126736) c5(126737, 126738, 126739, 126740, 126741, 126742) c6( ) c7(T, wu, fd, ci, zU, Qp, eW, Na, nX, aT, rO, Nc, wm, nJ, aOX, d, s, vT, aW, ff, Az, en, iP, ck, apM, ac, anx, iu, BA); #c1(JAG2) c2(NP_660142) c3(protein jagged-2 isoform b precursor) c4(240381) c5(240382, 240383, 240384, 240385, 240386, 240387) c6( ) c7(T, wu, fd, ci, zU, Qp, eW, Na, nX, aT, rO, Nc, wm, nJ, aOX, d, s, vT, aW, ff, Az, en, iP, ck, apM, ac, anx, iu, BA); #c1(JAGN1) c2(NP_115881) c3(protein jagunal homolog 1) c4(224450, 224451) c5(224452, 224453, 224454, 224455, 224456, 224457, 224458, 224459, 224460, 224461, 224462, 224463) c6( ) c7(gV, afK); #c1(JAK2) c2(NP_004963) c3(tyrosine-protein kinase JAK2) c4(151501, 151502) c5(151503, 151504, 151505, 151506, 151507, 151508, 151509, 151510, 151511, 151512, 151513, 151514, 151515, 151516, 151517, 151518) c6( ) c7(lD, bM, T, os, aC, bTV, Js, ea, if, fd, bB, ci, ip, x, bpl, Bc, fm, Ds, K, hH, cp, ly, bpR, up, pr, aW, az, hl, mQ, HA, vs, as, MV, Fu, cm, bw, eM, iT, wE, oy, cj, wx, k, m, lS, aeA, P, Ro, aiu, dD, n, oY, fr, qN, bkU, br, ft, kH, jy, eV, hn, DU, ox, ux, aBl, Nr, OV, mR, A, sq, fe, po, pm, cM, apX, lx, yR, bTX, dB, se, kz, nL, pg, B, Jo, bL, Dw, jv, fM, dA, kA, eX, aA, sM, ol, bTW, j, v, aqf, V, q, bTY, eA, oT, dn, aT, cy, dz, bTZ, ae, af, VC, iU, oV, Y, r, gm, en, hf, t, f, ik, ck, ac, bt, gV, ow, df, jh, oZ, eq, jE, hF, ad, iC, qV, oW, iY, lQ, adx, nJ, pq, Dl, d, UT, hG, et, ff, cr, kD, af, iV, bz, ok, bUa, Nq, age, eU, pc, Ew, bE, ag, akC, gZ, aS, lC, X, sH, Tc); #c1(JAK3) c2(XP_005259953) c3(tyrosine-protein kinase JAK3 isoform X2) c4( ) c5(276320, 276321, 276322) c6( ) c7(oO, bM, T, dA, dK, e, if, jh, fd, bB, oY, aGx, ci, qN, PJ, hF, ip, x, no, kH, aT, vw, dz, dL, ey, K, iY, ji, pq, bAE, je, anP, iU, aW, Y, oV, CM, fe, aGy, aF, jg, jT, f, bw, ck, CU, iM, Dy, iT, bE, azK, cj, at, pg, CO, k, bt, zA, Cp); #c1(JAK3) c2(XP_006722808) c3(tyrosine-protein kinase JAK3 isoform X1) c4(294875, 294876) c5(294877, 294878, 294879, 294880, 294881, 294882, 294883, 294884, 294885, 294886, 294887, 294888, 294889) c6( ) c7(oO, bM, T, dA, dK, e, if, jh, fd, bB, oY, aGx, ci, qN, PJ, hF, ip, x, no, kH, aT, vw, dz, dL, ey, K, iY, ji, pq, bAE, je, anP, iU, aW, Y, oV, CM, fe, aGy, aF, jg, jT, f, bw, ck, CU, iM, Dy, iT, bE, azK, cj, at, pg, CO, k, bt, zA, Cp); #c1(JAK3) c2(XP_011526292) c3(tyrosine-protein kinase JAK3 isoform X3) c4(320559) c5( ) c6( ) c7(oO, bM, T, dA, dk, e, if, jh, fd, bB, oY, aGx, ci, qN, PJ, hF, ip, x, no, kH, aT, vw, dz, dL, ey, K, iY, ji, pq, bAE, je, anP, iU, aW, Y, oV, CM, fe, aGy, aF, jg, jT, f, bw, ck, CU, iM, Dy, iT, bE, azK, cj, at, pg, CO, k, bt, zA, Cp); #c1(JAK3) c2(XP_011526293) c3(tyrosine-protein kinase JAK3 isoform X4) c4( ) c5(320560, 320561, 320562, 320563, 320564, 320565) c6( ) c7(oO, bM, T, dA, dk, e, if, jh, fd, bB, oY, aGx, ci, qN, PJ, hF, ip, x, no, kH, aT, vw, dz, dL, ey, K, iY, ji, pq, bAE, je, anP, iU, aW, Y, oV, CM, fe, aGy, aF, jg, jT, f, bw, ck, CU, iM, Dy, lT, bE, azK, cj, at, pg, CO, k, bt, zA, Cp); #c1(JAKMIP1) c2(NP_001092903) c3(janus kinase and microtubule-interacting protein 1 isoform 1) c4(39750, 39751) c5(39752, 39753, 39754, 39755, 39756, 39757, 39758, 39759, 39760, 39761, 39762, 39763) c6( ) c7(oe, bN); #c1(JAKMIP2) c2(NP_001257863) c3(janus kinase and microtubule-interacting protein 2 isoform 2) c4(95676, 95677) c5(95678, 95679, 95680, 95681, 95682, 95683, 95684, 95685, 95686, 95687, 95688, 95689) c6( ) c7(nJ, T, it, hp, Y); #c1(JAKMIP2) c2(NP_001269211) c3(janus kinase and microtubule-interacting protein 2 isoform 4) c4(100794) c5(100795, 100796, 100797, 100798, 100799, 100800) c6( ) c7(nJ, T, it, hp, Y); #c1(JAKMIP2) c2(NP_055605) c3(janus kinase and microtubule-interacting protein 2 isoform 3) c4(182199) c5(182200, 182201, 182202, 182203, 182204, 182205) c6( ) c7(nJ, T, it, hp, Y); #c1(JAKMIP3) c2(XP_011537986) c3(janus kinase and microtubule-interacting protein 3 isoform X1) c4(335521) c5(335522, 335523, 335524, 335525, 335526, 335527) c6( ) c7(ac); #c1(JAM2) c2(NP_001257336) c3(junctional adhesion molecule B isoform 2 precursor) c4(95209) c5(95210, 95211, 95212, 95213, 95214, 95215) c6( ) c7(oe, eA); #c1(JAM2) c2(NP_001257337) c3(junctional adhesion molecule B isoform 3 precursor) c4(95216) c5(95217, 95218, 95219, 95220, 95221, 95222) c6( ) c7(oe, eA); #c1(JAM2) c2(NP_067042) c3(junctional adhesion molecule B isoform 1 precursor) c4(208573, 208574) c5(208575, 208576, 208577, 208578, 208579, 208580, 208581, 208582, 208583, 208584, 208585, 208586) c6( ) c7(oe, eA); #c1(JAM3) c2(NP_001192258) c3(junctional adhesion molecule C isoform 2 precursor) c4(82160) c5(82161, 82162, 82163, 82164, 82165, 82166) c6( ) c7(bUb, fM, dD, eM, sw, Y, My, fO, acA, n, bK, m, fD, eq, eA, t, ai, y); #c1(JAM3) c2(NP_116190) c3(junctional adhesion molecule C isoform 1 precursor) c4(225652, 225653) c5(225654, 225655, 225656, 225657, 225658, 225659, 225660, 225661, 225662, 225663, 225664, 225665) c6( ) c7(bUb, fM, dD, eM, sw, Y, My, fO, acA, n, bK, m, fD, eq, eA, t, ai, y); #c1(JARID2) c2(NP_001253969) c3(protein Jumanji isoform 2) c4( ) c5(94316, 94317, 94318, 94319, 94320) c6( ) c7(bv, T, VX, rz, lp, kH, cm, amj, pg, K, nA, bD, hH, cd, vQ); #c1(JARID2) c2(NP_004964) c3(protein Jumonji isoform 1) c4(151519, 151520) c5(151521, 151522, 151523, 151524, 151525, 151526, 151527, 151528, 151529, 151530, 151531, 151532, 151533, 151534) c6( ) c7(bv, T, VX, rz, lp, kH, cm, amj, pg, K, nA, bD, hH, cd, vQ); #c1(JARID2) c2(XP_011512881) c3(protein Jumanji isoform X2) c4(304248) c5(304249) c6( ) c7(bv, T, VX, rz, lp, kH, cm, amj, pg, K, nA, bD, hH, cd, vQ); #c1(JARID2) c2(XP_011512885) c3(protein Jumonji isoform X5) c4(304250) c5(304251, 304252, 304253, 304254, 304255, 304256) c6( ) c7(bv, T, VX, rz, lp, kH, cm, amj, pg, K, nA, bD, hH, cd, vQ); #c1(JARID2) c2(XP_011512886) c3(protein Jumanji isoform X4) c4( ) c5(304257, 304258, 304259, 304260) c6( ) c7(bv, T, VX, rz, lp, kH, cm, amj, pg, K, nA, bD, hH, cd, vQ); #c1(JAZF1) c2(NP_778231) c3(juxtaposed with another zinc finger protein 1) c4(251455) c5(251456, 251457, 251458, 251459, 251460, 251461) c6( ) c7(bL, T, oe, EQ, bB, aj, v, q, J, ad, h, ae, A, eZ, r, cl, aEm, t, pb, Qf, bE, hL, m, Th); #c1(JDP2) c2(XP_005267389) c3(jun dimerization protein 2 isoform X1) c4(280947, 280948) c5(280949, 280950, 280951, 280952, 280953, 280954, 280955, 280956, 280957, 280958, 280959, 280960, 280961, 280962, 280963, 280964, 280965) c6( ) c7(ck, aE, jh, y, bt, B, us); #c1(JDP2) c2(XP_006720095) c3(jun dimerization protein 2 isoform X2) c4(293240) c5(293241, 293242, 293243, 293244, 293245, 293246, 293247, 293248, 293249, 293250) c6( ) c7(ck, aE, jh, y, bt, B, us); #c1(JDP2) c2(XP_011534733) c3(jun dimerization protein 2 isoform X3) c4( ) c5(331199) c6( ) c7(ck, aE, jh, y, bt, B, us); #c1(JMJD1C) c2(NP_001269877) c3(probable JmjC domain-containing histone demethylation protein 2C isoform c) c4(103003) c5(103004, 103005, 103006, 103007, 103008, 103009, 103010, 103011, 103012, 103013, 103014, 103015) c6( ) c7(ck, x, T, aW, qK, fc, aT, aA, aF, bqS, k, afW, ei, bN); #c1(JMJD1C) c2(NP_116165) c3(probable JmjC domain-containing histone demethylation protein 2C isoform a) c4(225492) c5(225493, 225494, 225495, 225496, 225497, 225498, 225499, 225500) c6( ) c7(ck, x, T, aW, qK, fc, aT, aA, aF, bqS, k, afW, ei, bN); #c1(JMJD1C) c2(XP_011537807) c3(probable JmjC domain-containing histone demethylation protein 2C isoform X4) c4(335322) c5( ) c6( ) c7(ck, x, T, aW, qK, fc, aT, aA, af, bqS, k, afW, ei, bN); #c1(JMJD1C) c2(XP_011537810) c3(probable JmjC domain-containing histone demethylation protein 2C isoform X7) c4(335323) c5(335324, 335325, 335326, 335327, 335328) c6( ) c7(ck, x, T, aW, qK, fc, aT, aA, aF, bqS, k, afW, ei, bN); #c1(JMJD6) c2(NP_001074930) c3(bifunctional arginine demethylase and lysyl-hydroxylase JMJD6 isoform 1) c4(37134, 37135) c5(37136, 37137, 37138, 37139, 37140, 37141) c6( ) c7(T, dB, aW, bE, aT, bB, uz, qG, afq, g, iu, ao); #c1(JMJDG) c2(NP_055982) c3(bifunctional arginine demethylase and lysyl-hydroxylase JMJD6 isoform 2) c4(184864) c5(184865, 184866, 184867, 184868, 184869, 184870, 184871, 184872, 184873, 184874, 184875, 184876) c6( ) c7(T, dB, aW, bE, aT, bB, uz, qG, afq, g, iu, ao); #c1(JMY) c2(NP_689618) c3(junction-mediating and -regulatory protein) c4(243296, 243297) c5(243298, 243299, 243300, 243301, 243302, 243303, 243304, 243305, 243306, 243307, 243308, 243309) c6( ) c7(mR, K, aT); #c1(JPH2) c2(NP_787109) c3(junctophilin-2 isoform 2) c4( ) c5(252097, 252098, 252099, 252100, 252101, 252102) c6( ) c7(gZ, vc, cc, bUc); #c1(JPH2) c2(XP_006723895) c3(junctophilin-2 isoform X1) c4(295433, 295434) c5(295435, 295436, 295437, 295438, 295439, 295440) c6( ) c7(gZ, vc, cc, bUc); #c1(JPH3) c2(NP_001258533) c3(junctophilin-3 isoform 2) c4(96111) c5(96112, 96113, 96114, 96115, 96116, 96117, 96118) c6( ) c7(bL, dD, ea, aw, cz, v, j, eq, ec, Ak, ZU, jy, yZ, ZM, bc, bUd, Pw, ajG, xw, dR, rR, ac, c, kB, Fi, U, dy, aan); #c1(JPH3) c2(NP_065706) c3(junctophilin-3 isoform 1) c4(205011) c5(205012, 205013, 205014, 205015, 205016, 205017, 205018) c6( ) c7(bL, dD, ea, aw, cz, v, j, eq, ec, Ak, ZU, jy, yZ, ZM, bc, bUd, Pw, ajG, xw, dR, rR, ac, c, kB, Fi, U, dy, aan); #c1(JPH3) c2(XP_006721300) c3(junctophilin-3 isoform X1) c4(293933) c5(293934, 293935, 293936, 293937, 293938) c6( ) c7(bL, dD, ea, aw, cz, v, j, eq, ec, Ak, ZU, jy, yZ, ZM, bc, bUd, Pw, ajG, xw, dR, rR, ac, c, kB, Fi, U, dy, aan); #c1(JPH4) c2(NP_115828) c3(junctophilin-4) c4(224165, 224166) c5(224167, 224168, 224169, 224170, 224171, 224172, 224173, 224174, 224175, 224176, 224177, 224178, 224179, 224180, 224181, 224182, 224183, 224184, 224185, 224186) c6( ) c7(Ye); #c1(JRK) c2(NP_001070995) c3(jerky protein homolog isoform b) c4(35257) c5(35258, 35259, 35260, 35261, 35262, 35263) c6( ) c7(GZ, ha, HN, iD, hg, il); #c1(JRK) c2(NP_003715) c3(jerky protein homolog isoform a) c4(140376) c5(140377, 140378, 140379, 140380, 140381, 140382) c6( ) c7(GZ, ha, HN, iD, hg, il); #c1(JSRP1) c2(NP_653217) c3(junctional sarcoplasmic reticulum protein 1) c4(239143) c5(239144, 239145, 239146, 239147, 239148, 239149) c6( ) c7(hD, lW, kl, Ak); #c1(JTB) c2(NP_006685) c3(protein JTB precursor) c4(167135, 167136) c5(167137, 167138, 167139, 167140, 167141, 167142, 167143, 167144, 167145, 167146, 167147, 167148, 167149, 167150, 167151, 167152, 167153, 167154, 167155, 167156) c6( ) c7(gh, NK, T, cR, n, Sv, ci, mC, ad, hX, sN, aT, cy, ae, OV, aW, Y, eZ, kT, bE, cj, fL, y, B); #c1(JUNB) c2(NP_002220) c3(transcription factor jun-B) c4(126743) c5(126744, 126745, 126746, 126747, 126748, 126749, 126750) c6( ) c7(bM, T, if, e, hE, bB, ci, x, aE, K, eL, aa, eG, aW, az, eE, eM, nB, wB, m, k, qN, ai, h, eV, dL, anP, aq, pm, kT, GU, cB, B, vQ, NQ, fM, yk, eX, rf, aA, cz, hz, v, j, C, q, eA, nv, aT, dz, ae, af, bf, iU, Y, r, lo, tu, en, cl, t, iK, pb, iM, hc, CZ, fD, ao, kh, wi, Ke, qb, aD, jh, dG, ad, av, hd, Yi, gj, d, je, et, ff, cr, eZ, tv, aF, si, bz, ek, es, bE, aS, lC, iu, y); #c1(JUND) c2(NP_005345) c3(transcription factor jun-D isoform JunD-FL) c4(155274) c5(155275, 155276, 155277, 155278, 155279, 155280, 155281) c6( ) c7(bM, T, if, e, al, bB, ci, x, aE, K, eL, aa, eG, aW, az, eE, eM, nB, wB, k, m, qN, PJ, ai, kH, h, dL, anP, aq, kT, GU, cB, B, vQ, NQ, yk, rf, aA, cz, hz, v, C, q, eA, nv, aT, ae, af, bf, Y, r, cu, lo, tu, en, t, ik, pb, ck, iM, hc, CZ, fD, bt, ao, Ke, qb, aD, jh, dG, ad, hd, lC, Yi, gj, d, je, ff, cr, eZ, tv, aF, si, bz, ek, es, bE, aS, y, iu); #c1(JUP) c2(XP_006721941) c3(junction plakoglobin isoform X1) c4(294408, 294409, 294410, 294411, 294412, 294413, 294414) c5(294415, 294416, 294417, 294418, 294419, 294420, 294421, 294422, 294423, 294424, 294425, 294426, 294427, 294428, 294429, 294430, 294431, 294432, 294433, 294434, 294435, 294436, 294437, 294438, 294439, 294440, 294441, 294442, 294443, 294444, 294445, 294446, 294447, 294448, 294449, 294450, 294451, 294452, 294453, 294454, 294455, 294456, 294457, 294458, 294459, 294460, 294461, 294462, 294463, 294464, 294465, 294466, 294467, 294468, 294469, 294470) c6( ) c7(dD, aEj, ea, n, j, ci, eq, fr, q, ad, bnC, alD, cc, aT, dL, Qr, bUe, aW, ff, hl, r, biv, en, nB, wK, hL); #c1(KAL1) c2(NP_000207) c3(anosmin-1 precursor) c4(4592) c5(4593, 4594, 4595, 4596, 4597, 4598) c6( ) c7(T, Ut, lP, fd, bB, ti, bUg, v, nA, mF, yn, UE, wG, bti, nd, he, btr, ae, aMi, avP, d, bUf, oV, aVQ, aeS, yq, bUh, eE, t, aiv, dB, bE, m, UF, cE, B); #c1(KALRN) c2(NP_001019831) c3(kalirin isoform 1) c4(27930) c5(27931, 27932, 27933, 27934, 27935, 27936) c6( ) c7(sc, T, aA, cz, j, ci, Ea, q, ad, eA, dP, aT, h, af, eu, aW, Y, ff, r, Ej, aF, en, t, sK, fq, bz, cm, ck, rR, Dy, ac, aS, m, B); #c1(KALRN) c2(NP_003938) c3(kalirin isoform 2) c4(142332) c5(142333, 142334, 142335, 142336, 142337, 142338) c6( ) c7(sc, T, aA, cz, j, ci, Ea, q, ad, eA, dP, aT, h, af, eu, aW, Y, ff, r, Ej, aF, en, t, sK, fq, bz, cm, ck, rR, Dy, ac, aS, m, B); #c1(KALRN) c2(NP_008995) c3(kalirin isoform 3) c4(170120, 170121) c5(170122, 170123, 170124, 170125, 170126, 170127, 170128, 170129, 170130, 170131, 170132, 170133) c6( ) c7(sc, T, aA, cz, j, ci, Ea, q, ad, eA, dP, aT, h, af, eu, aW, Y, ff, r, Ej, aF, en, t, sK, fq, bz, cm, ck, rR, Dy, ac, aS, m, B); #c1(KALRN) c2(XP_006713873) c3(kalirin isoform X1) c4( ) c5(287412) c6( ) c7(sc, T, aA, cz, j, ci, Ea, q, ad, eA, dP, aT, h, af, eu, aW, Y, ff, r, Ej, aF, en, t, sK, fq, bz, cm, ck, rR, Dy, ac, aS, m, B); #c1(KALRN) c2(XP_006713880) c3(kalirin isoform X15) c4( ) c5(287413) c6( ) c7(sc, T, aA, cz, j, ci, Ea, q, ad, eA, dP, aT, h, af, eu, aW, Y, ff, r, Ej, aF, en, t, sK, fq, bz, cm, ck, rR, Dy, ac, aS, m, B); #c1(KALRN) c2(XP_011511587) c3(kalirin isoform X12) c4( ) c5(302321, 302322, 302323, 302324) c6( ) c7(sc, T, aA, cz, j, ci, Ea, q, ad, eA, dP, aT, h, af, eu, aW, Y, ff, r, Ej, aF, en, t, sK, fq, bz, cm, ck, rR, Dy, ac, aS, m, B); #c1(KALRN) c2(XP_011511588) c3(kalirin isoform X14) c4(302325) c5(302326, 302327, 302328, 302329, 302330, 302331) c6( ) c7(sc, T, aA, cz, j, ci, Ea, q, ad, eA, dP, aT, h, af, eu, aW, Y, ff, r, Ej, aF, en, t, sK, fq, bz, cm, ck, rR, Dy, ac, aS, m, B); #c1(KANK1) c2(NP_001243806) c3(KN motif and ankyrin repeat domain-containing protein 1 isoform a) c4(91763) c5(91764, 91765, 91766, 91767, 91768, 91769, 91770, 91771, 91772, 91773, 91774, 91775, 91776, 91777, 91778, 91779, 91780, 91781, 91782, 91783, 91784, 91785, 91786) c6( ) c7(bM, T, es, iA, aW, aT, kD, oR, axk, fr, pm, bUi, V); #c1(KANK1) c2(NP_694856) c3(KN motif and ankyrin repeat domain-containing protein 1 isoform b) c4(245397, 245398, 245399) c5(245400, 245401, 245402, 245403, 245404, 245405, 245406, 245407, 245408, 245409, 245410) c6( ) c7(bM, T, es, iA, aW, aT, kD, oR, axk, fr, pm, bUi, V); #c1(KANK1) c2(XP_011516127) c3(KN motif and ankyrin repeat domain-containing protein 1 isoform X5) c4(309347) c5(309348, 309349, 309350, 309351, 309352, 309353) c6( ) c7(bM, T, es, iA, aW, aT, kD, oR, axk, fr, pm, bUi, V); #c1(KANK1) c2(XP_011516128) c3(KN motif and ankyrin repeat domain-containing protein 1 isoform X6) c4(309354) c5( ) c6( ) c7(bM, T, es, iA, aW, aT, kD, oR, axk, fr, pm, bUi, V); #c1(KANK2) c2(NP_001129663) c3(KN motif and ankyrin repeat domain-containing protein 2 isoform 2) c4(52115) c5(52116, 52117, 52118, 52119, 52120, 52121, 52122, 52123, 52124, 52125) c6( ) c7(cc, aE, iA, aW, aA, aT, aF, fr, eA); #c1(KANK2) c2(NP_056308) c3(KN motif and ankyrin repeat domain-containing protein 2 isoform 1) c4(187021) c5(187022, 187023, 187024, 187025, 187026, 187027) c6( ) c7(cc, aE, iA, aW, aA, aT, aF, fr, eA); #c1(KANK4) c2(XP_011539148) c3(KN motif and ankyrin repeat domain-containing protein 4 isoform X3) c4(337222) c5(337223, 337224, 337225, 337226, 337227, 337228, 337229, 337230, 337231, 337232, 337233, 337234, 337235, 337236, 337237, 337238, 337239, 337240) c6( ) c7(y, mR); #c1(KANSL1) c2(XP_005275701) c3(KAT8 regulatory NSL complex subunit 1 isoform X1) c4(285704) c5(285705, 285706, 285707, 285708, 285709, 285710) c6( ) c7(bUj, bUk, hb, qK, Az, nA, an, lM, aYh); #c1(KANSL1) c2(XP_006721886) c3(KAT8 regulatory NSL complex subunit 1 isoform X1) c4(294366) c5(294367, 294368, 294369, 294370, 294371, 294372, 294373, 294374, 294375, 294376, 294377, 294378, 294379, 294380, 294381, 294382, 294383, 294384) c6( ) c7(bUj, bUk, hb, qK, Az, nA, an, lM, aYh); #c1(KANSL1) c2(XP_011522932) c3(KAT8 regulatory NSL complex subunit 1 isoform X4) c4(317435) c5(317436, 317437, 317438, 317439, 317440, 317441) c6( ) c7(bUj, bUk, hb, qK, Az, nA, an, lM, aYh); #c1(KANSL1) c2(XP_011544644) c3(KAT8 regulatory NSL complex subunit 1 isoform X4) c4(343755) c5(343756, 343757, 343758, 343759, 343760, 343761) c6( ) c7(bUj, bUk, hb, qK, Az, nA, an, lM, aYh); #c1(KANSL3) c2(NP_001108488) c3(KAT8 regulatory NSL complex subunit 3) c4(43256, 43257) c5( ) c6( ) c7(nw); #c1(KANSL3) c2(XP_005264044) c3(KAT8 regulatory NSL complex subunit 3 isoform X8) c4(278652, 278653) c5(278654, 278655, 278656, 278657, 278658, 278659, 278660, 278661, 278662) c6( ) c7(nw); #c1(KANSL3) c2(XP_011509762) c3(KAT8 regulatory NSL complex subunit 3 isoform X6) c4( ) c5(300318, 300319, 300320, 300321, 300322, 300323, 300324) c6( ) c7(nw); #c1(KANSL3) c2(XP_011509768) c3(KAT8 regulatory NSL complex subunit 3 isoform X12) c4(300325, 300326) c5(300327, 300328, 300329, 300330, 300331, 300332, 300333, 300334, 300335, 300336, 300337, 300338, 300339, 300340, 300341, 300342, 300343, 300344, 300345, 300346) c6( ) c7(nw); #c1(KARS) c2(NP_001123561) c3(lysine-tRNA ligase isoform 1) c4(47607) c5(47608, 47609, 47610, 47611, 47612, 47613) c6( ) c7(bUl, bUm, dt, aPR, ds, Pg, bUn, h, fu, bX, bU, kC); #c1(KARS) c2(NP_005539) c3(lysine-tRNA ligase isoform 2) c4(157241) c5(157242, 157243, 157244, 157245, 157246, 157247) c6( ) c7(bUl, bUm, dt, aPR, ds, Pg, bUn, h, fu, bX, bU, kC); #c1(KAT2A) c2(NP_066564) c3(histone acetyltransferase KAT2A) c4(207403) c5(207404, 207405, 207406, 207407, 207408, 207409) c6( ) c7(x, T, oG, aW, rU, U, aT, alw, dq, aA, k, bUo, bt); #c1(KAT2B) c2(NP_003875) c3(histone acetyltransferase KAT2B) c4(141712) c5(141713, 141714, 141715, 141716, (41717, 141718) c6( ) c7(bM, T, aA, bB, aj, j, Kx, J, x, bv, OG, KH, aT, bLo, jf, A, d, Jl, aW, rU, eZ, alw, dq, iy, aF, t, bE, U, Kj, k, it, B); #c1(KAT5) c2(NP_001193762) c3(histone acetyltransferase KAT5 isoform 4) c4(82543) c5(82544, 82545, 82546, 82547, 82548, 82549, 82550) c6( ) c7(oO, bM, T, os, dD, ea, fG, jh, Nt, bB, oZ, v, Kh, ci, C, auu, eq, V, q, aR, ad, wD, bUp, hd, h, cy, dL, K, ae, af, eL, cp, pi, je, sp, Y, r, dq, aq, bz, ek, ck, nM, iM, eM, nB, bE, z, U, ph, y, bt, KS, B); #c1(KAT5) c2(NP_006379) c3(histone acetyltransferase KAT5 isoform 2) c4(164499) c5(164500, 164501, 164502, 164503, 164504) c6( ) c7(oO, bM, T, os, dD, ea, fG, jh, Nt, bB, oZ, v, Kh, ci, C, auu, eq, V, q, aR, ad, wD, bUp, hd, h, cy, dL, K, ae, af, eL, cp, pi, je, sp, Y, r, dq, aq, bz, ek, ck, nM, iM, eM, nB, bE, z, U, ph, y, bt, kS, B); #c1(KAT5) c2(NP_874368) c3(histone acetyltransferase KAT5 isoform 3) c4(256643) c5(256644, 256645, 256646, 256647, 256648) c6( ) c7(oO, bM, T, os, dD, ea, fG, jh, Nt, bB, oZ, v, Kh, ci, C, auu, eq, V, q, aR, ad, wD, bUp, hd, h, cy, dL, K, ae, af, eL, cp, pi, je, sp, Y, r, dq, aq, bz, ek, ck, nM, iM, eM, nB, bE, z, U, ph, y, bt, kS, B); #c1(KAT5) c2(NP_874369) c3(histone acetyltransferase KAT5 isoform 1) c4(256649) c5(256650, 256651, 256652, 256653, 256654, 256655, 256656) c6( ) c7(oO, bM, T, os, dD, ea, fG, jh, Nt, bB, oZ, v, Kh, ci, C, auu, eq, V, q, aR, ad, wD, bUp, hd, h, cy, dL, K, ae, af, eL, cp, pi, je, sp, Y, r, dq, aq, bz, ek, ck, nM, iM, eM, nB, bE, z, U, ph, y, bt, KS, B); #c1(KAT6A) c2(NP_006757) c3(histone acetyltransferase KAT6A) c4(167753, 167754, 167755) c5(167756, 167757, 167758, 167759, 167760, 167761, 167762, 167763, 167764, 167765, 167766, 167767, 167768, 167769, 167770, 167771, 167772, 167773, 167774, 167775, 167776) c6( ) c7(ck, x, T, jf, iZ, jp, RX, vA, qK, gE, k, hF, bt, aVE); #c1(KAT6B) c2(NP_001243397) c3(histone acetyltransferase KAT6B isoform 2) c4(90664) c5(90665, 90666, 90667, 90668, 90669, 90670) c6( ) c7(ck, T, yS, e, vA, all, bUs, oi, bt, bUq, bUr); #c1(KAT6B) c2(NP_001243398) c3(histone acetyltransferase KAT6B isoform 3) c4(90671) c5(90672, 90673, 90674, 90675, 90676, 90677, 90678, 90679, 90680, 90681, 90682, 90683, 90684, 90685) c6( ) c7(ck, T, yS, e, vA, all, bUs, oi, bt, bUq, bUr); #c1(KAT6B) c2(XP_005269721) c3(histone acetyltransferase KAT6B isoform X1) c4(282727) c5(282728, 282729, 282730, 282731, 282732, 282733, 282734, 282735, 282736, 282737, 282738) c6( ) c7(ck, T, yS, e, vA, all, bUs, oi, bt, bUq, bUr); #c1(KAT7) c2(NP_001186084) c3(histone acetyltransferase KAT7 isoform 2) c4(77057, 77058) c5(77059, 77060, 77061, 77062, 77063, 77064, 77065, 77066, 77067, 77068, 77069, 77070) c6( ) c7(aW, T, aT); #c1(KAT7) c2(NP_001186085) c3(histone acetyltransferase KAT7 isoform 3) c4(77071) c5(77072, 77073, 77074, 77075, 77076, 77077) c6( ) c7(aW, T, aT); #c1(KAT7) c2(NP_001186086) c3(histone acetyltransferase KAT7 isoform 4) c4(77078) c5(77079, 77080, 77081, 77082, 77083, 77084) c6( ) c7(aW, T, aT); #c1(KAT7) c2(NP_001186087) c3(histone acetyltransferase KAT7 isoform 5) c4(77085) c5(77086, 77087, 77088, 77089, 77090, 77091) c6( ) c7(aW, T, aT); #c1(KAT7) c2(NP_008998) c3(histone acetyltransferase KAT7 isoform 1) c4(170141, 170142) c5(170143, 170144, 170145, 170146, 170147, 170148, 170149, 170150, 170151, 170152, 170153, 170154, 170155) c6( ) c7(aW, T, aT); #c1(KAT8) c2(NP_115564) c3(histone acetyltransferase KAT8 isoform 1) c4(223201) c5(223202, 223203, 223204, 223205, 223206, 223207) c6( ) c7(jf, es, eM, aW, Rr, aA, aF, fr, C, bz, q); #c1(KAT8) c2(NP_892003) c3(histone acetyltransferase KAT8 isoform 2) c4(257443) c5(257444, 257445, 257446, 257447, 257448, 257449) c6( ) c7(jf, es, eM, aW, Rr, aA, aF, fr, C, bz, q); #c1(KATNA1) c2(NP_001191005) c3(katanin p60 ATPase-containing subunit A1 isoform 2) c4(80504) c5(80505, 80506, 80507, 80508, 80509, 80510) c6( ) c7(aT); #c1(KATNA1) c2(XP_005266862) c3(katanin p60 ATPase-containing subunit A1 isoform X2) c4( ) c5(280364, 280365, 280366, 280367, 280368, 280369) c6( ) c7(aT); #c1(KATNA1) c2(XP_011533699) c3(katanin p60 ATPase-containing subunit A1 isoform X1) c4(329749, 329750, 329751) c5(329752, 329753, 329754, 329755, 329756, 329757, 329758, 329759, 329760, 329761, 329762, 329763, 329764, 329765, 329766, 329767) c6( ) c7(aT); #c1(KATNAL2) c2(XP_005258414) c3(katanin p60 ATPase-containing subunit A-like 2 isoform X1) c4(275655, 275656) c5(275657) c6( ) c7(beA, bN); #c1(KATNAL2) c2(XP_006722617) c3(katanin p60 ATPase-containing subunit A-like 2 isoform X3) c4( ) c5(294768, 294769, 294770, 294771, 294772, 294773, 294774, 294775, 294776, 294777, 294778) c6( ) c7(beA, bN); #c1(KATNAL2) c2(XP_011524522) c3(katanin p60 ATPase-containing subunit A-like 2 isoform X5) c4(319091) c5(319092, 319093, 319094, 319095, 319096, 319097) c6( ) c7(beA, bN); #c1(KATNB1) c2(NP_005877) c3(katanin p80 WD40 repeat-containing subunit B1) c4(160074) c5(160075, 160076, 160077, 160078, 160079, 160080) c6( ) c7(qt, Db, T, aT); #c1(KAZALD1) c2(XP_005270251) c3(kazal-type serine protease inhibitor domain-containing protein 1 isoform X2) c4( ) c5(283291, 283292, 283293, 283294, 283295, 283296, 283297, 283298) c6(283299, 283300) c7(arY, bjl, nO); #c1(KAZALD1) c2(XP_011538525) c3(kazal-type serine protease inhibitor domain-containing protein 1 isoform X1) c4(336272, 336273) c5(336274, 336275, 336276, 336277, 336278, 336279, 336280, 336281) c6(336282, 336283) c7(arY, bjl, nO); #c1(KAZN) c2(XP_011539377) c3(kazrin isoform X2) c4(337485) c5( ) c6( ) c7(oe, j); #c1(KAZN) c2(XP_011539382) c3(kazrin isoform X7) c4(337486) c5(337487, 337488, 337489, 337490, 337491, 337492) c6( ) c7(oe, j); #c1(KBTBD11) c2(NP_055682) c3(kelch repeat and BTB domain-containing protein 11) c4(182754) c5(182755, 182756, 182757, 182758, 182759, 182760) c6( ) c7(an); #c1(KBTBD13) c2(NP_001094832) c3(kelch repeat and BTB domain-containing protein 13) c4(40772) c5(40773, 40774, 40775, 40776, 40777, 40778) c6( ) c7(Am, but); #c1(KBTBD8) c2(NP_115894) c3(kelch repeat and BTB domain-containing protein 8) c4(224549, 224550) c5(224551, 224552, 224553, 224554, 224555, 224556, 224557, 224558, 224559, 224560, 224561, 224562) c6( ) c7(aw); #c1(KCMF1) c2(NP_064507) c3(E3 ubiquitin-protein ligase KCMF1) c4(202687) c5(202688, 202689, 202690, 202691, 202692, 202693) c6( ) c7(T, aF, aE, aA); #c1(KCNA1) c2(NP_000208) c3(potassium voltage-gated channel subfamily A member 1) c4(4599) c5(4600, 4601, 4602, 4603, 4604, 4605, 4606, 4607, 4608, 4609) c6( ) c7(T, wY, aVR, ha, lr, akg, eA, aR, aym, lu, ayk, aT, K, akr, bUu, jf, Ng, alp, aW, KE, kB, Gp); #c1(KCNA4) c2(NP_002224) c3(potassium voltage-gated channel subfamily A member 4) c4(126751) c5(126752, 126753, 126754, 126755, 126756, 126757) c6( ) c7(bv, T, hM, cV, vc, K, EZ, pd, HO, mu); #c1(KCNA5) c2(NP_002225) c3(potassium voltage-gated channel subfamily A member 5) c4(126758) c5(126759, 126760, 126761, 126762, 126763, 126764) c6( ) c7(HQ, bM, T, lD, mr, wL, al, eX, cV, fZ, aA, cz, aj, jh, j, Fc, eA, tO, ad, J, BY, ga, aT, eV, vc, K, bq, ae, HO, mu, wC, ly, d, dH, aW, aF, EZ, as, pb, lx, bUv, eM, lC); #c1(KCNA6) c2(XP_011519257) c3(potassium voltage-gated channel subfamily A member 6 isoform X1) c4(313903) c5(313904, 313905, 313906, 313907, 313908, 313909, 313910, 313911, 313912, 313913) c6( ) c7(aQp); #c1(KCNA7) c2(NP_114092) c3(potassium voltage-gated channel subfamily A member 7) c4(222135) c5(222136, 222137, 222138, 222139, 222140, 222141) c6( ) c7(bL, bUw); #c1(KCNAB1) c2(NP_003462) c3(voltage-gated potassium channel subunit beta-l isoform 2) c4(138533) c5(138534, 138535, 138536, 138537, 138538, 138539) c6( ) c7(Hx, nX, ha); #c1(KCNAB1) c2(NP_751891) c3(voltage-gated potassium channel subunit beta-1 isoform 3) c4(248224) c5(248225, 248226, 248227, 248228, 248229, 248230) c6( ) c7(Hx, nX, ha); #c1(KCNAB1) c2(NP_751892) c3(voltage-gated potassium channel subunit beta-l isoform 1) c4(248231) c5(248232, 248233, 248234, 248235, 248236, 248237) c6( ) c7(Hx, nX, ha); #c1(KCNAB2) c2(XP_011540622) c3(voltage-gated potassium channel subunit beta-2 isoform X2) c4(339153) c5( ) c6( ) c7(ha, bO, bS, ct, Z, bln, cC); #c1(KCNAB2) c2(XP_011540626) c3(voltage-gated potassium channel subunit beta-2 isoform X5) c4( ) c5(339154, 339155, 339156, 339157, 339158, 339159, 339160, 339161, 339162, 339163, 339164, 339165, 339166, 339167, 339168, 339169, 339170, 339171) c6( ) c7(ha, bO, bS, ct, Z, bln, cC); #c1(KCNAB3) c2(NP_004723) c3(voltage-gated potassium channel subunit beta-3) c4(149082) c5(149083, 149084, 149085, 149086, 149087, 149088) c6( ) c7(uu); #c1(KCNB1) c2(XP_011527101) c3(potassium voltage-gated channel subfamily B member 1 isoform X1) c4(321590, 321591) c5(321592, 321593, 321594, 321595, 321596, 321597, 321598, 321599, 321600, 321601, 321602, 321603) c6( ) c7(bL, jf, ac, wk, hL, cz, lm, K, fu, cl, cd, mu); #c1(KCNB2) c2(NP_004761) c3(potassium voltage-gated channel subfamily B member 2) c4(149386) c5(149387, 149388, 149389, 149390, 149391, 149392) c6( ) c7(vo); #c1(KCNC1) c2(NP_001106212) c3(potassium voltage-gated channel subfamily C member 1 isoform 1) c4(42405) c5(42406, 42407, 42408, 42409, 42410, 42411) c6( ) c7(yU, yZ); #c1(KCNC1) c2(NP_004967) c3(potassium voltage-gated channel subfamily C member 1 isoform 2) c4(151535) c5(151536, 151537, 151538, 151539, 151540, 151541) c6( ) c7(yU, yZ); #c1(KCNC2) c2(NP_001247427) c3(potassium voltage-gated channel subfamily C member 2 isoform KV3.2d) c4(93570) c5(93571, 93572, 93573, 93574, 93575, 93576, 93577, 93578, 93579, 93580) c6( ) c7(T); #c1(KCNC2) c2(NP_631874) c3(potassium voltage-gated channel subfamily C member 2 isoform KV3.2a) c4(238173) c5(238174, 238175, 238176, 238177, 238178, 238179, 238180, 238181, 238182, 238183) c6( ) c7(T); #c1(KCNC2) c2(NP_631875) c3(potassium voltage-gated channel subfamily C member 2 isoform KV3.2b) c4(238184) c5(238185, 238186, 238187, 238188, 238189, 238190) c6( ) c7(T); #c1(KCNC2) c2(NP_715624) c3(potassium voltage-gated channel subfamily C member 2 isoform KV3.2c) c4(246677) c5(246678, 246679, 246680, 246681, 246682, 246683) c6( ) c7(T); #c1(KCNC3) c2(XP_006723266) c3(potassium voltage-gated channel subfamily C member 3 isoform X1) c4(295065) c5(295066, 295067, 295068, 295069, 295070, 295071) c6( ) c7(alN, Ks, ky, kB, U, dr, nA, bUx, aR, wT); #c1(KCNC3) c2(XP_011525227) c3(potassium voltage-gated channel subfamily C member 3 isoform X2) c4( ) c5(319689) c6( ) c7(alN, Ks, ky, kB, U, dr, nA, bUx, aR, wT); #c1(KCNC3) c2(XP_011525230) c3(potassium voltage-gated channel subfamily C member 3 isoform X3) c4(319690, 319691) c5(319692, 319693, 319694, 319695, 319696, 319697, 319698, 319699, 319700, 319701, 319702, 319703) c6( ) c7(alN, Ks, ky, kB, U, dr, nA, bUx, aR, wT); #c1(KCND2) c2(NP_036413) c3(potassium voltage-gated channel subfamily D member 2 precursor) c4(173651) c5(173652, 173653, 173654, 173655, 173656, 173657) c6( ) c7(ha, cz, ajM, Kw, il, bN, mu); #c1(KCNE1) c2(NP_001257334) c3(potassium voltage-gated channel subfamily E member 1) c4(95188) c5(95189, 95190, 95191, 95192, 95193, 95194, 95195, 95196, 95197, 95198, 95199, 95200, 95201, 95202, 95203, 95204, 95205, 95206, 95207, 95208) c6( ) c7(bL, T, bzy, Fj, mr, wL, e, ha, hx, cV, EQ, aw, rf, ajM, atN, il, cd, cc, h, adm, K, HO, mu, A, vj, bUy, bUz, EZ, Fh, gZ); #c1(KCNE2) c2(NP_751951) c3(potassium voltage-gated channel subfamily E member 2) c4(248611) c5(248612, 248613, 248614, 248615, 248616, 248617, 248618) c6( ) c7(bL, mr, aD, wL, hx, aw, cV, aA, ajM, afv, j, atN, Mk, tO, cc, oW, bq, bUB, bUA, HO, mu, s, aF, EZ, vo, bUC, lM, gT); #c1(KCNE3) c2(NP_005463) c3(potassium voltage-gated channel subfamily E member 3) c4(156383, 156384) c5(156385, 156386, 156387, 156388, 156389, 156390, 156391, 156392, 156393, 156394, 156395, 156396, 156397, 156398, 156399, 156400, 156401, 156402, 156403, 156404, 156405, 156406, 156407, 156408, 156409, 156410, 156411) c6( ) c7(bUD, lJ, ayl, lg, Fh, ayB, wL, ayH, VU, aPm, blp, tO, HT, mu); #c1(KCNE3) c2(XP_011543015) c3(potassium voltage-gated channel subfamily E member 3 isoform X1) c4(341706, 341707, 341708) c5(341709, 341710, 341711, 341712, 341713, 341714, 341715) c6( ) c7(bUD, lJ, ayl, lg, Fh, ayB, wL, ayH, VU, aPm, blp, tO, HT, mu); #c1(KCNE4) c2(NP_542402) c3(potassium voltage-gated channel subfamily E member 4) c4(232366) c5(232367, 232368, 232369, 232370, 232371, 232372) c6( ) c7(wL, oe, hn, fq); #c1(KCNE5) c2(NP_036414) c3(potassium voltage-gated channel subfamily E member 1-like protein) c4(173658) c5(173659, 173660, 173661, 173662, 173663, 173664) c6( ) c7(arD, mu, ajM, wL, mr); #c1(KCNG3) c2(NP_579875) c3(potassium voltage-gated channel subfamily G member 3 isoform 1) c4(234676) c5(234677, 234678, 234679, 234680, 234681, 234682) c6( ) c7(T, ff, en); #c1(KCNG3) c2(NP_758847) c3(potassium voltage-gated channel subfamily G member 3 isoform 2) c4(249272) c5(249273, 249274, 249275, 249276, 249277, 249278) c6( ) c7(T, ff, en); #c1(KCNG4) c2(NP_758857) c3(potassium voltage-gated channel subfamily G member 4) c4(249279) c5(249280, 249281, 249282, 249283, 249284, 249285) c6( ) c7(ff, uu, en); #c1(KCNH1) c2(NP_002229) c3(potassium voltage-gated channel subfamily H member 1 isoform 2) c4(126765) c5(126766, 126767, 126768, 126769, 126770, 126771) c6( ) c7(T, if, ha, aA, qN, eA, aT, Dz, mu, d, s, qt, aW, Y, ff, eH, bUE, hf, en, ef, fq, ac, cj, gZ, im, bt); #c1(KCNH1) c2(NP_758872) c3(potassium voltage-gated channel subfamily H member 1 isoform 1) c4(249349) c5(249350, 249351, 249352, 249353, 249354, 249355) c6( ) c7(T, if, ha, aA, qN, eA, aT, Dz, mu, d, s, qt, aW, Y, ff, eH, bUE, hf, en, eF, fq, ac, cj, gZ, im, bt); #c1(KCNH2) c2(NP_000229) c3(potassium voltage-gated channel subfamily H member 2 isoform a) c4(5258) c5(5259, 5260, 5261, 5262, 5263, 5264, 5265) c6( ) c7(SJ, T, atj, e, EQ, bB, ci, x, bv, SV, Ou, K, hm, bnQ, g, as, cm, QA, iT, wK, mx, bjY, QF, nw, m, k, cZ, cV, n, cd, Sw, afR, ft, cc, hw, bUF, Zs, gq, h, hn, bl, dd, bnO, db, HO, pl, dx, ajV, Yl, AT, RT, AQ, W, hL, bUG, bL, bzy, mr, wL, nx, hx, rf, aA, cz, ajM, j, Mk, q, adm, avt, Fg, ae, jf, iF, s, oV, r, gm, en, cl, t, nC, f, ik, ck, aJt, bnR, bUH, np, OL, bt, rA, ao, Dd, ha, bnS, bhL, Fb, Gn, atN, hF, ad, vJ, Sr, iY, mu, d, lR, qt, ff, bgh, gl, aF, EZ, Tp, Z, bz, ayj, dr, qa, bgg, EW, bnP, bE, gZ, X); #c1(KCNH2) c2(NP_001191727) c3(potassium voltage-gated channel subfamily H member 2 isoform d) c4( ) c5(81620, 81621, 81622, 81623, 81624, 81625, 81626) c6( ) c7(SJ, T, atj, e, EQ, bB, ci, x, bv, SV, Ou, K, hm, bnQ, g, as, cm, QA, iT, wK, mx, bjY, QF, nw, m, k, cZ, cV, n, cd, Sw, afR, ft, cc, hw, bUF, Zs, gq, h, hn, bl, dd, bnO, db, HO, pl, dx, ajV, Yl, AT, RT, AQ, W, hL, BUG, bL, bzy, mr, wL, nx, hx, rf, aA, cz, ajM, j, Mk, q, adm, avt, Fg, ae, jf, if, s, oV, r, gm, en, cl, t, nC, f, ik, ck, aJt, bnR, bUH, np, OL, bt, rA, ao, Dd, ha, bnS, bhL, Fb, Gn, atN, hF, ad, vJ, Sr, iY, mu, d, lR, qt, ff, bgh, gl, aF, EZ, Tp, Z, bz, ayj, dr, qa, bgg, EW, bnP, bE, gZ, X); #c1(KCNH2) c2(NP_742053) c3(potassium voltage-gated channel subfamily H member 2 isoform b) c4(247893) c5(247894, 247895, 247896, 247897, 247898, 247899, 247900, 247901) c6( ) c7(SJ, T, atj, e, EQ, bB, ci, x, bv, SV, Ou, K, hm, bnQ, g, as, cm, QA, iT, wK, mx, bjY, QF, nw, m, k, cZ, cV, n, cd, Sw, afR, ft, cc, hw, bUF, Zs, gq, h, hn, bl, dd, bnO, db, HO, pl, dx, ajV, Yl, AT, RT, AQ, W, hL, bUG, bL, bzy, mr, wL, nx, hx, rf, aA, cz, ajM, j, Mk, q, adm, avt, Fg, ae, jf, if, s, oV, r, gm, en, cl, t, nC, f, ik, ck, alt, bnR, bUH, np, OL, bt, rA, ao, Dd, ha, bnS, bhL, Fb, Gn, atN, hF, ad, vJ, Sr, iY, mu, d, lR, qt, ff, bgh, gl, aF, EZ, Tp, Z, bz, ayj, dr, qa, bgg, EW, bnP, bE, gZ, X); #c1(KCNH2) c2(NP_742054) c3(potassium voltage-gated channel subfamily H member 2 isoform c) c4(247902, 247903) c5(247904, 247905, 247906, 247907, 247908, 247909, 247910) c6( ) c7(SJ, T, atj, e, EQ, bB, ci, x, bv, SV, Ou, K, hm, bnQ, g, as, cm, DA, iT, wK, mx, bjY, QF, nw, m, k, cZ, cV, n, cd, Sw, afR, ft, cc, hw, bUF, Zs, gq, h, hn, bl, dd, bnO, db, HO, pl, dx, ajV, Yl, AT, RT, AQ, W, hL, bUG, bL, bzy, mr, wL, nx, hx, rf, aA, cz, ajM, j, Mk, q, adm, avt, Fg, ae, jf, iF, s, oV, r, gm, en, cl, t, nC, f, iK, ck, aJt, bnR, bUH, np, OL, bt, rA, ao, Dd, ha, bnS, bhL, Fb, Gn, atN, hF, ad, vJ, Sr, iY, mu, d, lR, qt, ff, bgh, gl, aF, EZ, Tp, Z, bz, ayj, dr, qa, bgg, EW, bnP, bE, gZ, X); #c1(KCNH2) c2(XP_011514487) c3(potassium voltage-gated channel subfamily H member 2 isoform X1) c4(307189) c5(307190, 307191, 307192, 307193, 307194, 307195) c6( ) c7(SJ, T, atj, e, EQ, bB, ci, x, bv, SV, Ou, K, hm, bnQ, g, as, cm, QA, iT, wK, mx, bjY, OF, nw, m, k, cZ, cV, n, cd, Sw, afR, ft, cc, hw, bUF, Zs, gq, h, hn, bl, dd, bnO, db, HO, pl, dx, ajV, Yl, AT, RT, AQ, W, hL, BUG, bL, bzy, mr, wL, nx, hx, rf, aA, cz, ajM, j, Mk, q, adm, avt, Fg, ae, jf, iF, s, oV, r, gm, en, cl, t, nC, f, ik, ck, aJt, bnR, bUH, np, OL, bt, rA, ao, Dd, ha, bnS, bhL, Fb, Gn, atN, hF, ad, vJ, Sr, iY, mu, d, lR, qt, ff, bgh, gl, aF, EZ, Tp, Z, bz, ayj, dr, qa, bgg, EW, bnP, bE, gZ, X); #c1(KCNH3) c2(NP_036416) c3(potassium voltage-gated channel subfamily H member 3) c4(173665) c5(173666, 173667, 173668, 173669, 173670, 173671) c6( ) c7(ck, ha); #c1(KCNH4) c2(NP_036417) c3(potassium voltage-gated channel subfamily H member 4) c4(173672) c5(173673, 173674, 173675, 173676, 173677, (73678) c6( ) c7(lD, dD, id, yS, ea, aA, n, eq, iJ, q, ad, mj, nd, aqk, kf, aT, K, ae, ly, ak, eG, d, aW, Y, eZ, r, nf, hN, g, aaO, cm, Ld, lx, U, yA, bt, B); #c1(KCNH5) c2(NP_647479) c3(potassium voltage-gated channel subfamily H member 5 isoform 1) c4(238670, 238671, 238672, 238673) c5(238674, 238675, 238676, 238677, 238678, 238679, 238680, 238681, 238682, 238683, 238684, 238685) c6( ) c7(jf, bN, ac, dY, Y, ff, ha, lm, en, g, aR, bA); #c1(KCNH5) c2(NP_758963) c3(potassium voltage-gated channel subfamily H member 5 isoform 3) c4( ) c5(249414, 249415, 249416, 249417, 249418, 249419, 249420, 249421, 249422, 249423, 249424, 249425) c6( ) c7(jf, bN, ac, dY, Y, ff, ha, lm, en, g, aR, bA); #c1(KCNH5) c2(NP_110406) c3(potassium voltage-gated channel subfamily H member 6 isoform 1) c4(220109) c5(220110, 220111, 220112, 220113, 220114, 220115, 220116, 220117, 220118, 220119) c6( ) c7(AM, cn, s, apx); #c1(KCNH6) c2(NP_775115) c3(potassium voltage-gated channel subfamily H member 6 isoform 2) c4(249639) c5(249640, 249641, 249642, 249643, 249644, 249645, 249646, 249647, 249648, 249649) c6( ) c7(AM, cn, s, apx); #c1(KCNH7) c2(NP_150375) c3(potassium voltage-gated channel subfamily H member 7 isoform 1) c4(228089) c5(228090, 228091, 228092, 228093, 228094, 228095) c6( ) c7(s, aw, fq, cr, bBp, V, ae, ad); #c1(KCNH7) c2(NP_775185) c3(potassium voltage-gated channel subfamily H member 7 isoform 2) c4(249657) c5(249658, 249659, 249660, 249661, 249662, 249663) c6( ) c7(s, aw, fq, cr, bBp, V, ae, ad); #c1(KCNH8) c2(NP_653234) c3(potassium voltage-gated channel subfamily H member 8) c4(239265) c5(239266, 239267, 239268, 239269, 239270, 239271, 239272) c6( ) c7(lD, T, dD, aC, id, yS, ea, jh, aA, eq, iJ, q, ad, mj, nd, aqk, kf, aT, K, ae, ly, ak, eG, d, aW, eZ, r, nf, hN, g, aaO, cm, Ld, lx, eM, U, yA, bt, B); #c1(KCNH8) c2(XP_011531646) c3(potassium voltage-gated channel subfamily H member 8 isoform X2) c4(327786) c5( ) c6( ) c7(lD, T, dD, aC, id, yS, ea, jh, aA, eq, iJ, q, ad, mj, nd, aqk, kf, aT, K, ae, ly, ak, eG, d, aW, eZ, r, nf, hN, g, aaO, cm, Ld, lx, eM, U, yA, bt, B); #c1(KCNH8) c2(XP_011531648) c3(potassium voltage-gated channel subfamily H member 8 isoform X4) c4( ) c5(327787, 327788, 327789, 327790, 327791) c6( ) c7(lD, T, dD, aC, id, yS, ea, jh, aA, eq, iJ, q, ad, mj, nd, aqk, kf, aT, K, ae, ly, ak, eG, d, aW, eZ, r, nf, hN, g, aaO, cm, Ld, lx, eM, U, yA, bt, B); #c1(KCNIP1) c2(NP_001030009) c3(Kv channel-interacting protein 1 isoform 1) c4(30837) c5(30838, 30839, 30840, 30841, 30842, 30843, 30844, 30845, 30846) c6(30847, 30848) c7(V, afu); #c1(KCNIP1) c2(NP_001030010) c3(Kv channel-interacting protein 1 isoform 3) c4(30849) c5(30850, 30851, 30852, 30853, 30854, 30855, 30856, 30857) c6(30858, 30859) c7(V, afu); #c1(KCNIP1) c2(NP_001265268) c3(Kv channel-interacting protein 1 isoform 5) c4(98069) c5(98070, 98071, 98072, 98073, 98074, 98075, 98076, 98077) c6(98078, 98079) c7(V, afu); #c1(KCNIP1) c2(NP_001265269) c3(Kv channel-interacting protein 1 isoform 4) c4( ) c5(98080, 98081, 98082, 98083, 98084, 98085, 98086) c6(98087, 98088) c7(V, afu); #c1(KCNIP1) c2(NP_055407) c3(Kv channel-interacting protein 1 isoform 2) c4(180881) c5(180882, 180883, 180884, 180885, 180886, 180887, 180888, 180889, 180890, 180891, 180892, 180893, 180894, 180895, 180896) c6(180897, 180898, 180899, 180900) c7(V, afu); #c1(KCNIP1) c2(XP_006714924) c3(Kv channel-interacting protein 1 isoform X2) c4(288308) c5( ) c6( ) c7(V, afu); #c1(KCNIP2) c2(NP_055406) c3(Kv channel-interacting protein 2 isoform 1) c4(180868) c5(180869, 180870, 180871, 180872, 180873, 180874, 180875, 180876, 180877, 180878) c6(180879, 180880) c7(cd); #c1(KCNIP2) c2(NP_775283) c3(Kv channel-interacting protein 2 isoform 2) c4(249753) c5(249754, 249755, 249756, 249757, 249758, 249759, 249760, 249761) c6(249762, 249763) c7(cd); #c1(KCNIP2) c2(NP_775284) c3(Kv channel-interacting protein 2 isoform 3) c4(249764, 249765) c5(249766, 249767, 249768, 249769, 249770, 249771, 249772, 249773, 249774, 249775, 249776, 249777, 249778, 249779, 249780, 249781) c6(249782, 249783, 249784, 249785) c7(cd); #c1(KCNIP2) c2(NP_775285) c3(Kv channel-interacting protein 2 isoform 4) c4(249786, 249787) c5(249788, 249789, 249790, 249791, 249792, 249793, 249794, 249795, 249796, 249797, 249798, 249799, 249800, 249801, 249802) c6(249803, 249804, 249805, 249806) c7(cd); #c1(KCNIP2) c2(NP_775286) c3(Kv channel-interacting protein 2 isoform 5) c4(249807) c5(249808, 249809, 249810, 249811, 249812, 249813, 249814, 249815) c6(249816, 249817) c7(cd); #c1(KCNIP2) c2(NP_775287) c3(Kv channel-interacting protein 2 isoform 6) c4(249818, 249819) c5(249820, 249821, 249822, 249823, 249824, 249825, 249826, 249827, 249828, 249829, 249830, 249831, 249832, 249833, 249834, 249835) c6(249836, 249837, 249838, 249839) c7(cd); #c1(KCNIP2) c2(NP_775289) c3(Kv channel-interacting protein 2 isoform 7) c4(249840) c5(249841, 249842, 249843, 249844, 249845, 249846, 249847, 249848) c6(249849, 249850) c7(cd); #c1(KCNIP3) c2(NP_001030086) c3(calsenilin isoform 2 precursor) c4(30911) c5(30912, 30913, 30914, 30915, 30916, 30917) c6( ) c7(ck, aW, ha, aT, cm, fb, af); #c1(KCNIP3) c2(NP_038462) c3(calsenilin isoform 1) c4(176518, 176519) c5(176520, 176521, 176522, 176523, 176524, 176525, 176526, 176527, 176528, 176529, 176530, 176531, 176532) c6( ) c7(ck, aW, ha, aT, cm, fb, af); #c1(KCNIP4) c2(NP_001030175) c3(Kv channel-interacting protein 4 isoform 5) c4(30965) c5(30966, 30967, 30968, 30969, 30970, 30971, 30972) c6( ) c7(bAO, bM, ac, c, hb, fr, k, fq, eu); #c1(KCNIP4) c2(NP_079497) c3(Kv channel-interacting protein 4 isoform 1) c4(218400, 218401) c5(218402, 218403, 218404, 218405, 218406, 218407, 218408, 218409) c6( ) c7(bAO, bM, ac, c, hb, fr, k, fq, eu); #c1(KCNIP4) c2(NP_671710) c3(Kv channel-interacting protein 4 isoform 2) c4(242109) c5(242110, 242111, 242112, 242113, 242114, 242115) c6( ) c7(bAO, bM, ac, c, hb, fr, k, fq, eu); #c1(KCNIP4) c2(NP_671711) c3(Kv channel-interacting protein 4 isoform 3) c4( ) c5(242116, 242117, 242118, 242119, 242120, 242121, 242122, 242123, 242124, 242125, 242126) c6( ) c7(bAO, bM, ac, c, hb, fr, k, fq, eu); #c1(KCNIP4) c2(NP_671712) c3(Kv channel-interacting protein 4 isoform 4) c4(242127) c5(242128, 242129, 242130, 242131, 242132, 242133) c6( ) c7(bAO, bM, ac, c, hb, fr, k, fq, eu); #c1(KCNIP4) c2(XP_005248247) c3(Kv channel-interacting protein 4 isoform X4) c4( ) c5(268279) c6( ) c7(bAO, bM, ac, c, hb, fr, k, fq, eu); #c1(KCNJ1) c2(NP_000211) c3(ATP-sensitive inward rectifier potassium channel 1 isoform a) c4(4620, 4621) c5(4622, 4623, 4624, 4625, 4626, 4627) c6( ) c7(bUl, Hd, bL, mj, ahS, Tl, s, pN, XD, eR, vj, oe, uO, cz, bKs, aj, afV, Xy, ah, nA, J); #c1(KCNJ1) c2(NP_722449) c3(ATP-sensitive inward rectifier potassium channel 1 isoform c) c4( ) c5(246713, 246714, 246715, 246716) c6( ) c7(bUl, Hd, bL, mj, ahS, Tl, s, pN, XD, eR, vj, oe, uO, cz, bKs, aj, afV, Xy, ah, nA, J); #c1(KCNJ1) c2(NP_722450) c3(ATP-sensitive inward rectifier potassium channel 1 isoform b) c4( ) c5(246717, 246718, 246719, 246720, 246721, 246722, 246723, 246724, 246725, 246726, 246727, 246728, 246729, 246730) c6( ) c7(bUl, Hd, bL, mj, ahS, Tl, s, pN, XD, eR, vj, oe, uO, cz, bKs, aj, afV, Xy, ah, nA, J); #c1(KCNJ10) c2(NP_002232) c3(ATP-sensitive inward rectifier potassium channel 10) c4(126791) c5(126792, 126793, 126794, 126795, 126796, 126797) c6( ) c7(bL, cZ, ha, cV, aj, ur, nA, il, V, qW, aR, J, bUJ, XD, aMT, afV, dd, bUK, rz, adx, cB, d, bwf, vs, aPz, ale, yX, Hw, c, mG, vy, nw, Bh); #c1(KCNJ11) c2(NP_000516) c3(ATP-sensitive inward rectifier potassium channel 11 isoform 1) c4(10889) c5(10890, 10891, 10892, 10893, 10894, 10895, 10896) c6( ) c7(bL, dD, eR, aVR, aZ, Hm, e, ha, My, aw, cz, n, aj, dG, j, v, eq, cd, q, J, me, mh, h, cy, jy, K, hg, bq, af, hm, mu, cC, kl, A, lW, zR, r, Mz, hp, mb, dr, hq, mi, bo, bUM, mg, gZ, bUL, mf, lS, ei); #c1(KCNJ11) c2(XP_006718289) c3(ATP-sensitive inward rectifier potassium channel 11 isoform X1) c4(291693) c5(291694, 291695, 291696, 291697, 291698) c6( ) c7(bL, dD, eR, aVR, aZ, Hm, e, ha, My, aw, cz, n, aj, dG, j, v, eq, cd, q, J, me, mh, h, cy, jy, K, hg, bq, af, hm, mu, cC, kl, A, lW, zR, r, Mz, hp, mb, dr, hq, mi, bo, bUM, mg, gZ, bUL, mf, lS, ei); #c1(KCNJ12) c2(NP_066292) c3(ATP-sensitive inward rectifier potassium channel 12) c4(207103) c5(207104, 207105, 207106, 207107, 207108, 207109, 207110, 207111, 207112, 207113) c6( ) c7(T, aQi, bAe, np, K, atN, mu); #c1(KCNJ13) c2(NP_001165887) c3(inward rectifier potassium channel 13 isoform 2) c4(67930) c5(67931, 67932, 67933, 67934, 67935) c6( ) c7(ac, bkx, Hq, bUN, bUP, pT, bUO); #c1(KCNJ13) c2(NP_001165888) c3(inward rectifier potassium channel 13 isoform 3) c4( ) c5(67936) c6( ) c7(ac, bkx, Hq, bUN, bUP, pT, bUO); #c1(KCNJ13) c2(NP_002233) c3(inward rectifier potassium channel 13 isoform 1) c4(126798) c5(126799, 126800, 126801, 126802, 126803, 126804, 126805, 126806, 126807, 126808) c6( ) c7(ac, bkx, Hq, bUN, bUP, pT, bUO); #c1(KCNJ14) c2(NP_037480) c3(ATP-sensitive inward rectifier potassium channel 14) c4(175745) c5(175746, 175747, 175748, 175749, 175750, 175751, 175752, 175753, 175754, 175755) c6( ) c7(FE); #c1(KCNJ15) c2(XP_011527863) c3(ATP-sensitive inward rectifier potassium channel 15 isoform X1) c4(322816, 322817, 322818, 322819, 322820, 322821, 322822, 322823, 322824) c5(322825, 322826, 322827, 322828, 322829, 322830, 322831, 322832, 322833, 322834, 322835, 322836, 322837, 322838, 322839, 322840, 322841, 322842, 322843, 322844, 322845, 322846, 322847, 322848, 322849, 322850, 322851, 322852, 322853, 322854, 322855, 322856, 322857, 322858, 322859, 322860, 322861, 322862, 322863, 322864, 322865, 322866, 322867, 322868, 322869, 322870, 322871, 322872, 322873, 322874, 322875, 322876, 322877, 322878, 322879, 322880, 322881, 322882, 322883, 322884, 322885, 322886, 322887, 322888, 322889, 322890, 322891, 322892, 322893, 322894, 322895, 322896, 322897, 322898, 322899, 322900, 322901, 322902, 322903, 322904, 322905, 322906, 322907, 322908, 322909, 322910, 322911, 322912, 322913, 322914, 322915, 322916, 322917) c6( ) c7(bL, f, mj, jy); #c1(KCNJ16) c2(NP_001278551) c3(inward rectifier potassium channel 16 isoform a) c4(109816) c5(109817, 109818, 109819, 109820) c6( ) c7(bUJ); #c1(KCNJ16) c2(XP_011523083) c3(inward rectifier potassium channel 16 isoform X1) c4( ) c5(317569, 317570, 317571, 317572, 317573, 317574, 317575, 317576, 317577, 317578, 317579, 317580, 317581, 317582) c6( ) c7(bUJ); #c1(KCNJ18) c2(NP_001181887) c3(inward rectifier potassium channel 18) c4(74387) c5(74388, 74389, 74390, 74391, 74392) c6( ) c7(lg, bUQ, lJ, HT); #c1(KCNJ18) c2(XP_005276976) c3(inward rectifier potassium channel 18 isoform X1) c4( ) c5(285747) c6( ) c7(lg, bUQ, lJ, HT); #c1(KCNJ2) c2(XP_011523081) c3(inward rectifier potassium channel 2 isoform X1) c4(317557) c5(317558, 317559, 317560, 317561, 317562, 317563, 317564, 317565, 317566, 317567, 317568) c6( ) c7(mr, wL, aw, bUR, cV, rf, amj, bAe, j, atN, ft, ayH, oj, K, lQ, Gc, HO, mu, Gf, dg, EZ, bQ, ac, ayl, bUS, aBB, lq, BP, HB, bD, Pk); #c1(KCNJ3) c2(NP_001247437) c3(G protein-activated inward rectifier potassium channel 1 isoform 2) c4( ) c5(93623, 93624, 93625, 93626, 93627, 93628) c6( ) c7(bL, av, T, mj, d, eM, aW, ha, cV, aT, bAe, ci, g, bHm, cm, eA); #c1(KCNJ3) c2(NP_002230) c3(G protein-activated inward rectifier potassium channel 1 isoform 1) c4(126772, 126773) c5(126774, 126775, 126776, 126777, 126778, 126779) c6( ) c7(bL, av, T, mj, d, eM, aW, ha, cV, aT, bAe, ci, g, bHm, cm, eA); #c1(KCNJ4) c2(NP_690607) c3(inward rectifier potassium channel 4) c4(244910) c5(244911, 244912, 244913, 244914, 244915, 244916) c6( ) c7(bL, vj, HC, Mz, an, qO); #c1(KCNJ5) c2(XP_011541112) c3(G protein-activated inward rectifier potassium channel 4 isoform X1) c4(339621) c5(339622, 339623, 339624, 339625, 339626, 339627, 339628, 339629, 339630, 339631, 339632, 339633, 339634, 339635) c6( ) c7(Fj, mr, wL, bUU, e, cV, cz, bUV, aj, v, ajM, atN, J, av, mj, ajv, iv, bUW, bHm, Fg, HO, mu, avH, s, yL, bbe, Hu, cm, qa, bLF, bUT, HB, ei); #c1(KCNJ6) c2(NP_002231) c3(G protein-activated inward rectifier potassium channel 2) c4(126780) c5(126781, 126782, 126783, 126784, 126785, 126786, 126787, 126788, 126789, 126790) c6( ) c7(bL, av, mj, Z, vj, Gs, HC, bD, bAe, bS, bK, K, nA, ct, pK, f); #c1(KCNJ8) c2(XP_005253415) c3(ATP-sensitive inward rectifier potassium channel 8 isoform X1) c4(272478, 272479) c5(272480, 272481, 272482, 272483, 272484, 272485, 272486, 272487, 272488, 272489, 272490, 272491, 272492, 272493, 272494, 272495) c6( ) c7(mr, ms, Bc, ha, mx, cz, mm, lG, gZ, vc, j, EZ, C, ajM); #c1(KCNJ9) c2(XP_006711368) c3(G protein-activated inward rectifier potassium channel 3 isoform X2) c4(285971) c5( ) c6( ) c7(bL, av, bUX, ha, aj, ur, J); #c1(KCNJ9) c2(XP_011507822) c3(G protein-activated inward rectifier potassium channel 3 isoform X1) c4( ) c5(297962, 297963, 297964, 297965, 297966, 297967) c6( ) c7(bL, av, bUX, ha, aj, ur, J); #c1(KCNK1) c2(NP_002236) c3(potassium channel subfamily K member 1) c4(126809, 126810, 126811) c5(126812, 126813, 126814, 126815, 126816, 126817, 126818, 126819, 126820, 126821, 126822, 126823) c6( ) c7(dD, ajM, eq, m, t, eA); #c1(KCNK1) c2(XP_011542486) c3(potassium channel subfamily K member 1 isoform X1) c4( ) c5(340974, 340975, 340976, 340977, 340978, 340979) c6( ) c7(dD, ajM, eq, m, t, eA); #c1(KCNK10) c2(NP_066984) c3(potassium channel subfamily K member 10 isoform 1 precursor) c4(208018) c5(208019, 208020, 208021, 208022, 208023, 208024) c6( ) c7(cm, T, aaw, eu); #c1(KCNK10) c2(NP_612190) c3(potassium channel subfamily K member 10 isoform 2) c4(236135) c5(236136, 236137, 236138, 236139, 236140, 236141) c6( ) c7(cm, T, aaw, eu); #c1(KCNK10) c2(NP_612191) c3(potassium channel subfamily K member 10 isoform 3) c4(236142) c5(236143, 236144, 236145, 236146, 236147, 236148) c6( ) c7(cm, T, aaw, eu); #c1(KCNK12) c2(NP_071338) c3(potassium channel subfamily K member 12) c4(211093) c5(211094, 211095, 211096, 211097, 211098, 211099, 211100) c6( ) c7(vo, af, qj); #c1(KCNK16) c2(NP_001128577) c3(potassium channel subfamily K member 16 isoform 1) c4(50306) c5(50307, 50308, 50309, 50310, 50311, 50312) c6( ) c7(bL, T); #c1(KCNK16) c2(NP_001128578) c3(potassium channel subfamily K member 16 isoform 3) c4(50313) c5(50314, 50315, 50316, 50317, 50318, 50319) c6( ) c7(bL, T); #c1(KCNK16) c2(NP_001128579) c3(potassium channel subfamily K member 16 isoform 4) c4(50320, 50321) c5(50322, 50323, 50324, 50325, 50326, 50327, 50328) c6( ) c7(bL, T); #c1(KCNK16) c2(NP_115491) c3(potassium channel subfamily K member 16 isoform 2) c4(222894) c5(222895, 222896, 222897, 222898, 222899, 222900) c6( ) c7(bL, T); #c1(KCNK16) c2(XP_011513237) c3(potassium channel subfamily K member 16 isoform X2) c4( ) c5(305467, 305468, 305469, 305470, 305471) c6( ) c7(bL, T); #c1(KCNK17) c2(NP_001128583) c3(potassium channel subfamily K member 17 isoform 2) c4(50343) c5(50344, 50345, 50346, 50347, 50348, 50349) c6( ) c7(bL, eu, ac, M); #c1(KCNK17) c2(NP_113648) c3(potassium channel subfamily K member 17 isoform 1) c4(221918) c5(221919, 221920, 221921, 221922, 221923, 221924) c6( ) c7(bL, eu, ac, M); #c1(KCNK18) c2(NP_862823) c3(potassium channel subfamily K member 18) c4(255765) c5(255766, 255767, 255768, 255769, 255770, 255771) c6( ) c7(cZ, T, xi, ayp, ur, uu, dd, bUY); #c1(KCNK2) c2(NP_001017424) c3(potassium channel subfamily K member 2 isoform a) c4(26486, 26487, 26488) c5(26489, 26490, 26491, 26492, 26493, 26494, 26495, 26496, 26497, 26498, 26499, 26500, 26501, 26502, 26503, 26504, 26505, 26506, 26507, 26508) c6( ) c7(il, T, dD, c, wO, eu, ha, bO, cy, gf, bS, ct, eq, Z, vo, ae, ad, cB); #c1(KCNK2) c2(NP_001017425) c3(potassium channel subfamily K member 2 isoform c) c4(26509, 26510) c5(26511, 26512, 26513, 26514, 26515, 26516, 26517, 26518, 26519, 26520, 26521) c6( ) c7(il, T, dD, c, wO, eu, ha, bO, cy, gf, bS, ct, eq, Z, vo, ae, ad, cB); #c1(KCNK2) c2(NP_055032) c3(potassium channel subfamily K member 2 isoform b) c4(178403) c5(178404, 178405, 178406, 178407, 178408, 178409) c6( ) c7(il, T, dD, c, wO, eu, ha, bO, cy, gf, bS, ct, eq, Z, vo, ae, ad, cB); #c1(KCNK2) c2(XP_011507826) c3(potassium channel subfamily K member 2 isoform X2) c4( ) c5(297968, 297969, 297970) c6( ) c7(il, T, dD, c, wO, eu, ha, bO, cy, gf, bS, ct, eq, Z, vo, ae, ad, cB); #c1(KCNK3) c2(NP_002237) c3(potassium channel subfamily K member 3) c4(126824, (26825) c5(126826, 126827, 126828, 126829, 126830, 126831, 126832) c6( ) c7(lD, ly, T, mr, iX, ha, cV, cz, vc, K, MA, ek, bUZ, EZ, cB, HO, lx, mu); #c1(KCNK3) c2(XP_005264350) c3(potassium channel subfamily K member 3 isoform X1) c4( ) c5(278824, 278825, 278826, 278827, 278828) c6( ) c7(lD, ly, T, mr, iX, ha, cV, cz, vc, K, MA, ek, bUZ, EZ, cB, HO, lx, mu); #c1(KCNK5) c2(NP_003731) c3(potassium channel subfamily K member 5) c4(140497) c5(140498, 140499, 140500, 140501, 140502, 140503) c6( ) c7(T, s, iU, aW, Fg, aT, V, cB); #c1(KCNK6) c2(NP_004814) c3(potassium channel subfamily K member 6) c4(150002) c5(150003, 150004, 150005, 150006, 150007, 150008) c6( ) c7(qb); #c1(KCNK9) c2(XP_011515404) c3(potassium channel subfamily K member 9 isoform X1) c4(308220) c5(308221, 308222, 308223, 308224, 308225, 308226, 308227, 308228, 308229, 308230, 308231, 308232, 308233, 308234) c6( ) c7(T, d, Mr, aW, Y, HN, aT, bVa, nA, Fg); #c1(KCNMA1) c2(NP_001154824) c3(calcium-activated potassium channel subunit alpha-1 isoform c) c4(61149, 61150) c5(61151, 61152, 61153, 61154, 61155, 61156, 61157, 61158, 61159, 61160, 61161, 61162) c6( ) c7(T, oe, ha, aw, cz, aj, v, fr, eA, ad, cF, aT, cy, HR, bVb, hg, qM, Fg, Df, ae, ajT, aW, Y, vj, eH, agF, cl, eF, xw, qa, bN, zV, ac, c, bnP, wk, U, hL, el, bzm, cB); #c1(KCNMA1) c2(NP_001154825) c3(calcium-activated potassium channel subunit alpha-1 isoform d) c4(61163) c5(61164, 61165, 61166, 61167, 61168, 61169) c6( ) c7(T, oe, ha, aw, cz, aj, v, fr, eA, ad, cF, aT, cy, HR, bVb, hg, qM, Fg, Df, ae, ajT, aW, Y, vj, eH, agF, cl, eF, xw, qa, bN, zV, ac, c, bnP, wk, U, hL, el, bzm, cB); #c1(KCNMA1) c2(NP_001258448) c3(calcium-activated potassium channel subunit alpha-1 isoform f) c4(96081) c5( ) c6( ) c7(T, oe, ha, aw, cz, aj, v, fr, eA, ad, cF, aT, cy, HR, bVb, hg, qM, Fg, Df, ae, ajT, aW, Y, vj, eH, agF, cl, eF, xw, qa, bN, zV, ac, c, bnP, wk, U, hL, el, bzm, cB); #c1(KCNMA1) c2(NP_002238) c3(calcium-activated potassium channel subunit alpha-1 isoform b) c4(126833) c5(126834, 126835, 126836, 126837, 126838, 126839) c6( ) c7(T, oe, ha, aw, cz, aj, v, fr, eA, ad, cF, aT, cy, HR, bVb, hg, qM, Fg, Df, ae, ajT, aW, Y, vj, eH, agF, cl, eF, xw, qa, bN, zV, ac, c, bnP, wk, U, hL, el, bzm, cB); #c1(KCNMA1) c2(XP_005269833) c3(calcium-activated potassium channel subunit alpha-1 isoform X21) c4(282898) c5( ) c6( ) c7(T, oe, ha, aw, cz, aj, v, fr, eA, ad, cF, aT, cy, HR, bVb, hg, qM, Fg, Df, ae, ajT, aW, Y, vj, eH, agF, cl, eF, xw, qa, bN, zV, ac, c, bnP, wk, U, hL, el, bzm, cB); #c1(KCNMA1) c2(XP_005269835) c3(calcium-activated potassium channel subunit alpha-l isoform X9) c4(282899) c5( ) c6( ) c7(T, oe, ha, aw, cz, aj, v, fr, eA, ad, cF, aT, cy, HR, bVb, hg, qM, Fg, Df, ae, ajT, aW, Y, vj, eH, agF, cl, eF, xw, qa, bN, zV, ac, c, bnP, wk, U, hL, el, bzm, cB); #c1(KCNMA1) c2(XP_005269838) c3(calcium-activated potassium channel subunit alpha-1 isoform X10) c4(282900, 282901) c5(282902, 282903, 282904, 282905, 282906, 282907, 282908, 282909, 282910, 282911, 282912, 282913, 282914, 282915, 282916, 282917, 282918, 282919) c6( ) c7(T, oe, ha, aw, cz, aj, v, fr, eA, ad, cF, aT, cy, HR, bVb, hg, qM, Fg, Df, ae, ajT, aW, Y, vj, eH, agF, cl, eF, xw, qa, bN, zV, ac, c, bnP, wk, U, hL, el, bzm, cB); #c1(KCNMA1) c2(XP_005269844) c3(calcium-activated potassium channel subunit alpha-1 isoform X22) c4(282920, 282921) c5(282922, 282923, 282924, 282925, 282926, 282927, 282928, 282929, 282930, 282931, 282932, 282933) c6( ) c7(T, oe, ha, aw, cz, aj, v, fr, eA, ad, cF, aT, cy, HR, bVb, hg, qM, Fg, Df, ae, ajT, aW, Y, vj, eH, agF, cl, eF, xw, qa, bN, zV, ac, c, bnP, wk, U, hL, el, bzm, cB); #c1(KCNMA1) c2(XP_005269846) c3(calcium-activated potassium channel subunit alpha-1 isoform X14) c4( ) c5(282934, 282935, 282936, 282937, 282938, 282939) c6( ) c7(T, oe, ha, aw, cz, aj, v, fr, eA, ad, cF, aT, cy, HR, bVb, hg, qM, Fg, Df, ae, ajT, aW, Y, vj, eH, agF, cl, eF, xw, qa, bN, zV, ac, c, bnP, wk, U, hL, el, bzm, cB); #c1(KCNMA1) c2(XP_005269853) c3(calcium-activated potassium channel subunit alpha-1 isoform X17) c4(282940) c5(282941, 282942, 282943, 282944, 282945, 282946) c6( ) c7(T, oe, ha, aw, cz, aj, v, fr, eA, ad, cF, aT, cy, HR, bVb, hg, qM, Fg, Df, ae, ajT, aW, Y, vj, eH, agF, cl, eF, xw, qa, bN, zV, ac, c, bnP, wk, U, hL, el, bzm, cB); #c1(KCNMA1) c2(XP_005269854) c3(calcium-activated potassium channel subunit alpha-1 isoform X18) c4(282947) c5(282948, 282949, 282950, 282951, 282952, 282953) c6( ) c7(T, oe, ha, aw, cz, aj, v, fr, eA, ad, cF, aT, cy, HR, bVb, hg, qM, Fg, Df, ae, ajT, aW, Y, vj, eH, agF, cl, eF, xw, qa, bN, zV, ac, c, bnP, wk, U, hL, el, bzm, cB); #c1(KCNMA1) c2(XP_011538081) c3(calcium-activated potassium channel subunit alpha-1 isoform X7) c4(335634, 335635) c5(335636, 335637, 335638, 335639, 335640, 335641, 335642, 335643, 335644, 335645, 335646, 335647) c6( ) c7(T, oe, ha, aw, cz, aj, v, fr, eA, ad, cF, aT, cy, HR, bVb, hg, qM, Fg, Df, ae, ajT, aW, Y, vj, eH, agF, cl, eF, xw, qa, bN, zV, ac, c, bnP, wk, U, hL, el, bzm, cB); #c1(KCNMA1) c2(XP_011538085) c3(calcium-activated potassium channel subunit alpha-1 isoform X13) c4(335648) c5(335649, 335650, 335651, 335652, 335653, 335654) c6( ) c7(T, oe, ha, aw, cz, aj, v, fr, eA, ad, cF, aT, cy, HR, bVb, hg, qM, Fg, Df, ae, ajT, aW, Y, vj, eH, agF, cl, eF, xw, qa, bN, zV, ac, c, bnP, wk, U, hL, el, bzm, cB); #c1(KCNMA1) c2(XP_011538086) c3(calcium-activated potassium channel subunit alpha-1 isoform X19) c4(335655) c5(335656, 335657, 335658, 335659, 335660, 335661) c6( ) c7(T, oe, ha, aw, cz, aj, v, fr, eA, ad, cF, aT, cy, HR, bVb, hg, qM, Fg, Df, ae, ajT, aW, Y, vj, eH, agF, cl, eF, xw, qa, bN, zV, ac, c, bnP, wk, U, hL, el, bzm, cB); #c1(KCNMA1) c2(XP_011538087) c3(calcium-activated potassium channel subunit alpha-1 isoform X23) c4( ) c5(335662, 335663, 335664, 335665, 335666, 335667) c6( ) c7(T, oe, ha, aw, cz, aj, v, fr, eA, ad, cF, aT, cy, HR, bVb, hg, qM, Fg, Df, ae, ajT, aW, Y, vj, eH, agF, cl, eF, xw, qa, bN, zV, ac, c, bnP, wk, U, hL, el, bzm, cB); #c1(KCNMB1) c2(NP_004128) c3(calcium-activated potassium channel subunit beta-1) c4(143805, 143806) c5(143807, 143808, 143809, 143810, 143811, 143812, 143813, 143814, 143815, 143816, 143817, 143818) c6( ) c7(fU, bM, ac, uW, vj, aw, cz, arD, bVc, dy, j, aY, eA); #c1(KCNMB2) c2(NP_001265840) c3(calcium-activated potassium channel subunit beta-2) c4(99301, 99302, 99303, 99304) c5(99305, 99306, 99307, 99308, 99309, 99310, 99311, 99312, 99313, 99314, 99315, 99316, 99317, 99318, 99319, 99320, 99321, 99322, 99323, 99324, 99325, 99326, 99327, 99328, 99329, 99330, 99331, 99332, 99333, 99334, 99335, 99336, 99337, 99338, 99339, 99340) c6( ) c7(hn, cd, bVd); #c1(KCNMB3) c2(NP_001157149) c3(calcium-activated potassium channel subunit beta-3 isoform e) c4(62076) c5(62077, 62078, 62079, 62080, 62081, 62082) c6( ) c7(yr, Cq, arl, ff, ha, HN, hg, en); #c1(KCNMB3) c2(NP_055222) c3(calcium-activated potassium channel subunit beta-3 isoform d) c4(179919) c5(179920, 179921, 179922, 179923, 179924, 179925) c6( ) c7(yr, Cq, arl, ff, ha, HN, hg, en); #c1(KCNMB3) c2(NP_741979) c3(calcium-activated potassium channel subunit beta-3 isoform a) c4(247492) c5(247493, 247494, 247495, 247496, 247497, 247498) c6( ) c7(yr, Cq, arl, ff, ha, HN, hg, en); #c1(KCNMB3) c2(NP_741980) c3(calcium-activated potassium channel subunit beta-3 isoform b) c4(247499) c5(247500, 247501, 247502, 247503, 247504, 247505, 247506, 247507, 247508, 247509) c6( ) c7(yr, Cq, arl, ff, ha, HN, hg, en); #c1(KCNMB3) c2(NP_741981) c3(calcium-activated potassium channel subunit beta-3 isoform c) c4(247510) c5(247511, 247512, 247513, 247514, 247515, 247516) c6( ) c7(yr, Cq, arl, ff, ha, HN, hg, en); #c1(KCNMB4) c2(NP_055320) c3(calcium-activated potassium channel subunit beta-4) c4(180455) c5(180456, 180457, 180458, 180459, 180460, 180461) c6( ) c7(nX, oe, ha); #c1(KCNN1) c2(NP_002239) c3(small conductance calcium-activated potassium channel protein 1) c4( ) c5(126840, 126841, 126842, 126843, 126844) c6( ) c7(iV); #c1(KCNN1) c2(XP_005259963) c3(small conductance calcium-activated potassium channel protein 1 isoform X2) c4(276323, 276324) c5(276325, 276326, 276327, 276328, 276329, 276330) c6( ) c7(IV); #c1(KCNN1) c2(XP_005259964) c3(small conductance calcium-activated potassium channel protein 1 isoform X4) c4( ) c5(276331) c6( ) c7(IV); #c1(KCNN2) c2(NP_067627) c3(small conductance calcium-activated potassium channel protein 2 isoform a) c4(209077, 209078) c5(209079, 209080, 209081, 209082, 209083, 209084, 209085, 209086, 209087, 209088, 209089) c6( ) c7(od, hb, e, aS, Xu, lf); #c1(KCNN2) c2(NP_740721) c3(small conductance calcium-activated potassium channel protein 2 isoform b) c4(247431) c5(247432, 247433, 247434, 247435, 247436) c6( ) c7(od, hb, e, aS, Xu, lf); #c1(KCNN2) c2(XP_011541691) c3(small conductance calcium-activated potassium channel protein 2 isoform X1) c4( ) c5(340218, 340219) c6( ) c7(od, hb, e, aS, Xu, lf); #c1(KCNN3) c2(NP_002240) c3(small conductance calcium-activated potassium channel protein 3 isoform a) c4(126845, 126846, 126847) c5(126848, 126849, 126850, 126851, 126852, 126853, 126854, 126855, 126856, 126857, 126858, 126859) c6( ) c7(T, iH, mr, wL, mS, afv, j, ur, uu, tO, mW, iX, aT, bK, mY, mV, on, mU, aW, Y, ahl, vo, mZ, dR, mX, mT); #c1(KCNN3) c2(NP_740752) c3(small conductance calcium-activated potassium channel protein 3 isoform b) c4(247455) c5(247456, 247457, 247458, 247459, 247460, 247461, 247462, 247463, 247464, 247465, 247466, 247467) c6( ) c7(T, iH, mr, wL, mS, afv, j, ur, uu, tO, mW, iX, aT, bK, mY, mV, on, mU, aW, Y, ahl, vo, mZ, dR, mX, mT); #c1(KCNN4) c2(NP_002241) c3(intermediate conductance calcium-activated potassium channel protein 4) c4(126860, 126861) c5(126862, 126863, 126864, 126865, 126866, 126867) c6( ) c7(bM, dD, ea, aw, cz, cR, bB, aj, eq, eA, J, El, ad, x, K, ae, Hl, cl, ek, LB, ck, bE, z, hL, hO, Hb); #c1(KCNN4) c2(XP_005258939) c3(intermediate conductance calcium-activated potassium channel protein 4 isoform X1) c4( ) c5(275902, 275903, 275904, 275905, 275906, 275907) c6( ) c7(bM, dD, ea, aw, cz, cR, bB, aj, eq, eA, J, El, ad, x, K, ae, Hl, cl, ek, LB, ck, bE, z, hL, hO, Hb); #c1(KCNQ1) c2(NP_000209) c3(potassium voltage-gated channel subfamily KQT member 1 isoform 1) c4(4610) c5(4611, 4612, 4613, 4614, 4615, 4616, 4617, 4618, 4619) c6( ) c7(bL, T, bzy, bVe, mr, wL, Or, e, ha, hx, EQ, cV, cz, bB, aj, v, j, bD, ml, atN, ajM, q, J, Mk, tO, er, iu, aw, cc, oW, h, jy, vc, adm, K, on, HO, mu, Qe, aa, bVg, vj, r, EZ, g, Z, Hl, bz, dr, eM, bVf, bUC, mG, ali, gX, mg, gZ, mx, avl, bo, dy, QF, lM, gq, rf); #c1(KCNQ1) c2(NP_861463) c3(potassium voltage-gated channel subfamily KQT member 1 isoform 2 precursor) c4(255609) c5(255610, 255611, 255612, 255613, 255614, 255615) c6( ) c7(bL, T, bzy, bVe, mr, wL, Or, e, ha, hx, EQ, cV, cz, bB, aj, v, j, bD, ml, atN, ajM, q, J, Mk, to, er, iu, aw, cc, oW, h, jy, vc, adm, K, on, HO, mu, Qe, aa, bVg, vj, r, EZ, g, Z, Hl, bz, dr, eM, bVf, bUC, mG, ali, gX, mg, gZ, mx, avl, bo, dy, QF, lM, gq, rf); #c1(KCNQ2) c2(NP_004509) c3(potassium voltage-gated channel subfamily KAT member 2 isoform c) c4(147019) c5(147020, 147021, 147022, 147023, 147024, 147025, 147026, 147027, 147028, 147029, 147030, 147031, 147032) c6( ) c7(bVk, ha, cV, mS, iD, nA, FR, aR, bVi, hb, mW, nX, lm, bK, hg, mY, mV, mu, mU, Kp, akf, mZ, akq, mX, bVh, Cq, bVj, aOc, avl, agT, mT); #c1(KCNQ2) c2(NP_742104) c3(potassium voltage-gated channel subfamily KQT member 2 isoform b) c4(248122, 248123) c5(248124, 248125, 248126, 248127, 248128, 248129, 248130) c6( ) c7(bVk, ha, cV, mS, iD, nA, FR, aR, bVi, hb, mW, nX, lm, bK, hg, mY, mV, mu, mU, Kp, akf, mZ, akq, mX, bVh, Cq, bVj, aOc, avl, agT, mT); #c1(KCNQ2) c2(NP_742105) c3(potassium voltage-gated channel subfamily KQT member 2 isoform a) c4(248131, 248132) c5(248133, 248134, 248135, 248136, 248137, 248138, 248139, 248140, 248141, 248142, 248143, 248144, 248145) c6( ) c7(bVk, ha, cV, mS, iD, nA, FR, aR, bVi, hb, mW, nX, lm, bK, hg, mY, mV, mu, mU, Kp, akf, mZ, akq, mX, bVh, Cq, bVj, aOc, avl, agT, ml); #c1(KCNQ2) c2(NP_742106) c3(potassium voltage-gated channel subfamily KAT member 2 isoform d) c4(248146) c5(248147, 248148, 248149, 248150, 248151, 248152, 248153, 248154, 248155, 248156, 248157, 248158) c6( ) c7(bVk, ha, cV, mS, iD, nA, FR, aR, bVi, hb, mW, nX, lm, bK, hg, mY, mV, mu, mU, Kp, akf, mZ, akq, mX, bVh, Cq, bVj, aOc, avl, agT, mT); #c1(KCNQ2) c2(NP_742107) c3(potassium voltage-gated channel subfamily KAT member 2 isoform e) c4(248159) c5(248160, 248161, 248162, 248163, 248164, 248165, 248166) c6( ) c7(bVk, ha, cV, mS, iD, nA, FR, aR, bVi, hb, mW, nX, lm, bK, hg, mY, mV, mu, mU, Kp, akf, mZ, akq, mX, bVh, Cq, bVj, aOc, avl, agT, mT); #c1(KCNQ2) c2(XP_006723850) c3(potassium voltage-gated channel subfamily KAT member 2 isoform X5) c4(295408) c5(295409, 295410, 295411, 295412, 295413, 295414) c6( ) c7(bVk, ha, cV, mS, iD, nA, FR, aR, bVi, hb, mW, nX, lm, bK, hg, mY, mV, mu, mU, Kp, akf, mZ, akq, mX, bVh, Cq, bVj, aOc, avl, agT, mT); #c1(KCNQ2) c2(XP_011527111) c3(potassium voltage-gated channel subfamily KQT member 2 isoform X3) c4(321604) c5( ) c6( ) c7(bVk, ha, cV, mS, iD, nA, FR, aR, bVi, hb, mW, nX, lm, bK, hg, mY, mV, mu, mU, Kp, akf, mZ, akq, mX, bVh, Cq, bVj, aOc, avl, agT, mT); #c1(KCNQ2) c2(XP_011527113) c3(potassium voltage-gated channel subfamily KQT member 2 isoform X6) c4(321605, 321606, 321607) c5(321608, 321609, 321610, 321611, 321612, 321613, 321614, 321615, 321616, 321617, 321618, 321619, 321620, 321621, 321622, 321623, 321624, 321625) c6( ) c7(bVk, ha, cV, mS, iD, nA, FR, aR, bVi, hb, mW, nX, lm, bK, hg, mY, mV, mu, mU, Kp, akf, mZ, akq, mX, bVh, Cq, bVj, aOc, avl, agT, mT); #c1(KCNQ3) c2(NP_001191753) c3(potassium voltage-gated channel subfamily KQT member 3 isoform 2 precursor) c4(81656, 81657) c5(81658, 81659, 81660, 81661, 81662, 81663, 81664, 81665, 81666, 81667, 81668, 81669) c6( ) c7(bVk, Cq, ac, ha, HN, akf, iD, avl, hg, nA, il, vo, agT, cd, akq); #c1(KCNQ3) c2(NP_004510) c3(potassium voltage-gated channel subfamily KAT member 3 isoform 1) c4(147033, 147034) c5(147035, 147036, 147037, 147038, 147039, 147040, 147041, 147042, 147043, 147044, 147045, 147046) c6( ) c7(bVk, Cq, ac, ha, HN, akf, iD, avl, hg, nA, il, vo, agT, cd, akq); #c1(KCNQ5) c2(NP_001153602) c3(potassium voltage-gated channel subfamily KAT member 5 isoform 2) c4(60714) c5(60715, 60716, 60717, 60718, 60719, 60720) c6( ) c7(atL, T, ha, Be); #c1(KCNQ5) c2(NP_001153604) c3(potassium voltage-gated channel subfamily KQT member 5 isoform 3) c4(60721) c5(60722, 60723, 60724, 60725, 60726, 60727) c6( ) c7(atL, T, ha, Be); #c1(KCNQ5) c2(NP_001153605) c3(potassium voltage-gated channel subfamily KQT member 5 isoform 4) c4(60728, 60729, 60730) c5(60731, 60732, 60733, 60734, 60735, 60736, 60737, 60738, 60739, 60740, 60741, 60742) c6( ) c7(atL, T, ha, Be); #c1(KCNQ5) c2(NP_001153606) c3(potassium voltage-gated channel subfamily KAT member 5 isoform 5) c4(60743) c5(60744, 60745, 60746, 60747, 60748, 60749, 60750, 60751, 60752, 60753, 60754, 60755) c6( ) c7(atL, T, ha, Be); #c1(KCNQ5) c2(NP_062816) c3(potassium voltage-gated channel subfamily KAT member 5 isoform 1) c4(202103, 202104, 202105) c5(202106, 202107, 202108, 202109, 202110, 202111, 202112, 202113, 202114, 202115, 202116, 202117, 202118, 202119, 202120, 202121, 202122, 202123) c6( ) c7(atL, T, ha, Be); #c1(KCNRG) c2(NP_775876) c3(potassium channel regulatory protein isoform 1) c4(250542) c5(250543, 250544, 250545, 250546, 250547, 250548) c6( ) c7(fd, B, ad, jh, cj); #c1(KCNRG) c2(NP_955751) c3(potassium channel regulatory protein isoform 2) c4(261939) c5(261940, 261941, 261942, 261943, 261944, 261945) c6( ) c7(fd, B, ad, jh, cj); #c1(KCNS1) c2(XP_005260466) c3(potassium voltage-gated channel subfamily S member 1 isoform X1) c4(276555) c5(276556, 276557, 276558, 276559, 276560, 276561, 276562, 276563, 276564, 276565) c6( ) c7(Pt, gf, h, hq); #c1(KCNS3) c2(XP_011531127) c3(potassium voltage-gated channel subfamily S member 3 isoform X1) c4(327092, 327093) c5(327094, 327095, 327096, 327097, 327098, 327099, 327100, 327101, 327102, 327103, 327104, 327105, 327106, 327107, 327108, 327109) c6( ) c7(vo, fq, bK, ac, c); #c1(KCNT1) c2(NP_065873) c3(potassium channel subfamily T member 1 isoform 1) c4(206002, 206003) c5(206004, 206005, 206006, 206007, 206008, 206009, 206010, 206011, 206012, 206013, 206014, 206015) c6( ) c7(bVm, bVl, ha, lr, aNX, hg, fq, aNW); #c1(KCNT1) c2(XP_011517182) c3(potassium channel subfamily T member 1 isoform X4) c4(310773) c5(310774, 310775, 310776, 310777, 310778, 310779) c6( ) c7(bVm, bVl, ha, lr, aNX, hg, fq, aNW); #c1(KCNT2) c2(NP_001274748) c3(potassium channel subfamily T member 2 isoform 2) c4(106750, 106751) c5(106752, 106753, 106754, 106755, 106756, 106757) c6( ) c7(W); #c1(KCNT2) c2(NP_001274749) c3(potassium channel subfamily T member 2 isoform 3) c4(106758) c5(106759, 106760, 106761, 106762, 106763, 106764) c6( ) c7(W); #c1(KCNT2) c2(NP_940905) c3(potassium channel subfamily T member 2 isoform 1) c4(260076) c5(260077, 260078, 260079, 260080, 260081, 260082, 260083, 260084, 260085, 260086, 260087, 260088, 260089) c6( ) c7(W); #c1(KCNT2) c2(XP_006711357) c3(potassium channel subfamily T member 2 isoform X1) c4( ) c5(285936, 285937) c6( ) c7(W); #c1(KCNT2) c2(XP_006711358) c3(potassium channel subfamily T member 2 isoform X2) c4(285938) c5( ) c6( ) c7(W); #c1(KCNT2) c2(XP_011507785) c3(potassium channel subfamily T member 2 isoform X4) c4( ) c5(297851, 297852, 297853, 297854, 297855) c6( ) c7(W); #c1(KCNU1) c2(NP_001027006) c3(potassium channel subfamily U member 1) c4(30062) c5(30063, 30064, 30065, 30066, 30067, 30068) c6( ) c7(cd); #c1(KCNV1) c2(NP_055194) c3(potassium voltage-gated channel subfamily V member 1) c4(179682, 179683) c5(179684, 179685, 179686, 179687, 179688, 179689, 179690, 179691, 179692, 179693, 179694, 179695, 179696, 179697, 179698, 179699) c6( ) c7(De); #c1(KCNV2) c2(NP_598004) c3(potassium voltage-gated channel subfamily V member 2) c4(235268, 235269) c5(235270, 235271, 235272, 235273, 235274, 235275, 235276, 235277, 235278, 235279, 235280, 235281) c6( ) c7(lR, nx, bVn, rf, OL, oQ, HO, mu); #c1(KCTD1) c2(NP_001136202) c3(BTB/POZ domain-containing protein KCTD1 isoform b) c4(54513, 54514) c5(54515, 54516, 54517, 54518, 54519, 54520) c6( ) c7(bVp, Xt, cj, bVo); #c1(KCTD1) c2(NP_001245150) c3(BTB/POZ domain-containing protein KCTD1 isoform a) c4( ) c5(92619, 92620, 92621, 92622, 92623, 92624, 92625, 92626, 92627, 92628, 92629) c6( ) c7(bVp, Xt, cj, bVo); #c1(KCTD1) c2(NP_001245151) c3(BTB/POZ domain-containing protein KCTD1 isoform c) c4( ) c5(92630, 92631, 92632) c6( ) c7(bVp, Xt, cj, bVo); #c1(KCTD1D) c2(NP_114160) c3(BTB/POZ domain-containing adapter for CUL3-mediated RhoA degradation protein 3) c4(222441, 222442, 222443) c5(222444, 222445, 222446, 222447, 222448, 222449, 222450, 222451, 222452, 222453, 222454, 222455, 222456, 222457, 222458, 222459, 222460, 222461, 222462, 222463, 222464, 222465, 222466, 222467) c6( ) c7(dQ, cm, fb); #c1(KCTD11) c2(NP_001002914) c3(BTB/POZ domain-containing protein KCTD11) c4(19763) c5(19764, 19765, 19766, 19767, 19768, 19769) c6( ) c7(jf, T, ft, ib, nO, oW, cz, iv, hS); #c1(KCTD12) c2(NP_612453) c3(BTB/POZ domain-containing protein KCTD12) c4(236523) c5(236524, 236525, 236526, 236527, 236528, 236529, 236530, 236531, 236532, 236533, 236534) c6( ) c7(bL, fb, j); #c1(KCTD13) c2(NP_849194) c3(BTB/POZ domain-containing adapter for CUL3-mediated RhoA degradation protein 1) c4(254489) c5(254490, 254491, 254492, 254493, 254494, 254495, 254496, 254497, 254498, 254499) c6( ) c7(QK, bN); #c1(KCTD15) c2(NP_076981) c3(BTB/POZ domain-containing protein KCTD15 isoform 1) c4(214571) c5(214572, 214573, 214574, 214575, 214576, 214577) c6( ) c7(bL, dD, ea, e, r, v, eq, fq, q); #c1(KCTD15) c2(XP_011525598) c3(BTB/POZ domain-containing protein KCTD15 isoform X2) c4(319989) c5(319990, 319991, 319992, 319993, 319994, 319995, 319996, 319997, 319998, 319999, 320000) c6( ) c7(bL, dD, ea, e, r, v, eq, fq, q); #c1(KCTD15) c2(XP_011525600) c3(BTB/POZ domain-containing protein KCTD15 isoform X1) c4(320001, 320002) c5(320003, 320004, 320005, 320006, 320007, 320008, 320009, 320010, 320011, 320012, 320013, 320014, 320015) c6( ) c7(bL, dD, ea, e, r, v, eq, fq, q); #c1(KCTD16) c2(XP_005268550) c3(BTB/POZ domain-containing protein KCTD16 isoform X1) c4(281883) c5(281884, 281885, 281886, 281887, 281888, 281889, 281890, 281891, 281892, 281893) c6( ) c7(j); #c1(KCTD2) c2(NP_056168) c3(BTB/POZ domain-containing protein KCTD2) c4(186081, 186082) c5(186083, 186084, 186085, 186086, 186087, 186088, 186089, 186090, 186091, 186092, 186093, 186094) c6( ) c7(aaw); #c1(KCTD21) c2(XP_005273982) c3(BTB/POZ domain-containing protein KCTD21 isoform X2) c4(284900, 284901, 284902) c5(284903, 284904) c6( ) c7(jf); #c1(KCTD21) c2(XP_006718579) c3(BTB/POZ domain-containing protein KCTD21 isoform X3) c4( ) c5(291905, 291906, 291907, 291908) c6( ) c7(jf); #c1(KCTD21) c2(XP_006718581) c3(BTB/POZ domain-containing protein KCTD21 isoform X1) c4(291909, 291910, 291911) c5(291912, 291913, 291914, 291915, 291916, 291917, 291918, 291919, 291920, 291921, 291922, 291923, 291924, 291925, 291926, 291927, 291928, 291929, 291930, 291931, 291932, 291933, 291934, 291935, 291936, 291937, 291938, 291939, 291940, 291941) c6( ) c7(jf); #c1(KCTD3) c2(NP_057205) c3(BTB/POZ domain-containing protein KCTD3) c4(189470, 189471) c5(189472, 189473, 189474, 189475, 189476, 189477) c6( ) c7(rz, ey); #c1(KCTD3) c2(XP_005273213) c3(BTB/POZ domain-containing protein KCTD3 isoform X1) c4(284337) c5(284338, 284339, 284340, 284341, 284342, 284343, 284344, 284345, 284346, 284347, 284348, 284349, 284350) c6( ) c7(rz, ey); #c1(KCTD3) c2(XP_005273215) c3(BTB/POZ domain-containing protein KCTD3 isoform X2) c4(284351, 284352) c5(284353, 284354, 284355, 284356, 284357, 284358, 284359, 284360, 284361, 284362, 284363) c6( ) c7(rz, ey); #c1(KCTD6) c2(NP_699162) c3(BTB/POZ domain-containing protein KCTD6) c4(245760) c5(245761, 245762, 245763, 245764, 245765, 245766, 245767, 245768, 245769, 245770, 245771, 245772, 245773, 245774, 245775, 245776, 245777, 245778) c6( ) c7(jf); #c1(KCTD7) c2(NP_001161433) c3(BTB/POZ domain-containing protein KCTD7 isoform 2) c4(65784) c5(65785, 65786, 65787, 65788, 65789, 65790) c6( ) c7(bEQ, bVq, yZ, bVr, yU, agT, rY); #c1(KCTD7) c2(NP_694578) c3(BTB/POZ domain-containing protein KCTD7 isoform 1) c4(245309) c5(245310, 245311, 245312, 245313, 245314, 245315) c6( ) c7(bEQ, bVq, yZ, bVr, yU, agT, rY); #c1(KCTD9) c2(NP_060104) c3(BTB/POZ domain-containing protein KCTD9) c4(194175) c5(194176, 194177, 194178, 194179, 194180, 194181) c6( ) c7(aGj, jE, vi); #c1(KDELC1) c2(NP_076994) c3(KDEL motif-containing protein 1 precursor) c4(214666) c5(214667, 214668, 214669, 214670, 214671, 214672) c6( ) c7(ac); #c1(KDELR1) c2(NP_006792) c3(ER lumen protein-retaining receptor 1) c4(168100) c5(168101, 168102, 168103, 168104, 168105, 168106) c6( ) c7(ad); #c1(KDM2A) c2(NP_001243334) c3(lysine-specific demethylase ZA isoform b) c4(90416, 90417) c5(90418, 90419, 90420, 90421, 90422, 90423, 90424, 90425, 90426, 90427, 90428, 90429, 90430) c6( ) c7(T, bz, ci, eM); #c1(KDM2A) c2(NP_036440) c3(lysine-specific demethylase 2A isoform a) c4(173885, 173886) c5(173887, 173888, 173889, 173890, 173891, 173892, 173893, 173894, 173895, 173896, 173897, 173898) c6( ) c7(T, bz, ci, eM); #c1(KDM2A) c2(XP_011543162) c3(lysine-specific demethylase 2A isoform X2) c4(341995) c5( ) c6( ) c7(T, bz, ci, eM); #c1(KDM2A) c2(XP_011543164) c3(lysine-specific demethylase 2A isoform X5) c4( ) c5(341996, 341997, 341998, 341999, 342000) c6( ) c7(T, bz, ci, eM); #c1(KDM2B) c2(NP_001005366) c3(lysine-specific demethylase 2B isoform b) c4(21497) c5(21498, 21499, 21500, 21501, 21502, 21503, 21504, 21505, 21506) c6( ) c7(ck, T, kP, aE, aW, aT, kp, anN, avQ); #c1(KDM2B) c2(NP_115979) c3(lysine-specific demethylase 2B isoform a) c4(225050, 225051) c5(225052, 225053, 225054, 225055, 225056, 225057, 225058, 225059, 225060, 225061, 225062, 225063) c6( ) c7(ck, T, kP, aE, aW, aT, kp, anN, avQ); #c1(KDM2B) c2(XP_005254013) c3(lysine-specific demethylase 2B isoform X12) c4(272867) c5( ) c6( ) c7(ck, T, kP, aE, aW, aT, kp, anN, avQ); #c1(KDM2B) c2(XP_011537170) c3(lysine-specific demethylase 2B isoform X9) c4(334926, 334927) c5( ) c6( ) c7(ck, T, kP, aE, aW, aT, kp, anN, avQ); #c1(KDM2B) c2(XP_011537171) c3(lysine-specific demethylase 2B isoform X10) c4( ) c5(334928, 334929, 334930, 334931, 334932, 334933, 334934, 334935, 334936, 334937, 334938, 334939) c6( ) c7(ck, T, kP, aE, aW, aT, kp, anN, avQ); #c1(KDM2B) c2(XP_011537 (72) c3(lysine-specific demethylase 2B isoform X13) c4( ) c5(334940, 334941, 334942, 334943, 334944, 334945) c6( ) c7(ck, T, kP, aE, aW, aT, kp, anN, avQ); #c1(KDM2B) c2(XP_011537177) c3(lysine-specific demethylase 2B isoform X18) c4(334946) c5(334947, 334948, 334949, 334950, 334951, 334952) c6( ) c7(ck, T, kP, aE, aW, aT, kp, anN, avQ); #c1(KDM3A) c2(NP_060903) c3(lysine-specific demethylase 3A) c4(198517, 198518, 198519, 198520) c5(198521, 198522, 198523, 198524, 198525, 198526, 198527, 198528, 198529, 198530, 198531, 198532, 198533, 198534, 198535, 198536, 198537, 198538, 198539, 198540, 198541, 198542, 198543, 198544, 198545, 198546, 198547, 198548, 198549, 198550, 198551, 198552, 198553, 198554, 198555, 198556, 198557, 198558, 198559, 198560, 198561, 198562, 198563, 198564, 198565) c6( ) c7(T, wu, bB, n, v, ci, q, ad, Pl, vH, dL, wm, ae, eL, ND, r, cm, bz, es, iA, bE, eD, B); #c1(KDM3A) c2(XP_011531287) c3(lysine-specific demethylase 3A isoform X1) c4(327262, 327263) c5(327264) c6( ) c7(T, wu, bB, n, v, ci, q, ad, Pl, vH, dL, wm, ae, eL, ND, r, cm, bz, es, iA, bE, eD, B); #c1(KDM3B) c2(NP_057688) c3(lysine-specific demethylase 3B) c4(192340, 192341, 192342, 192343) c5(192344, 192345, 192346, 192347, 192348, 192349, 192350, 192351, 192352, 192353, 192354, 192355, 192356, 192357, 192358, 192359, 192360, 192361, 192362) c6( ) c7(ck, x, iT, aW, r, aT, n, k, bdj, bt, q, hq); #c1(KDM3B) c2(XP_011541791) c3(lysine-specific demethylase 3B isoform X2) c4( ) c5(340383, 340384, 340385, 340386, 340387, 340388) c6( ) c7(ck, x, iT, aW, r, aT, n, k, bdj, bt, q, hq); #c1(KDM4B) c2(NP_055830) c3(lysine-specific demethylase 4B) c4(183759, 183760) c5(183761, 183762, 183763, 183764, 183765, 183766, 183767, 183768) c6(183769, 183770) c7(jf, ae, T, bE, aW, r, aT, aA, bB, dL, aF, g, bz, q, ad, bA); #c1(KDM4B) c2(XP_005259578) c3(lysine-specific demethylase 48 isoform X1) c4(276144) c5(276145, 276146, 276147, 276148, 276149, 276150) c6( ) c7(jf, ae, T, bE, aW, r, aT, aA, bB, dL, aF, g, bz, q, ad, bA); #c1(KDM4B) c2(XP_011526117) c3(lysine-specific demethylase 4B isoform X3) c4( ) c5(320394) c6( ) c7(jf, ae, T, bE, aW, r, aT, aA, bB, dL, aF, g, bz, q, ad, bA); #c1(KDM4B) c2(XP_011526120) c3(lysine-specific demethylase 48 isoform X7) c4( ) c5(320395, 320396, 320397, 320398, 320399, 320400) c6( ) c7(jf, ae, T, bE, aW, r, aT, aA, bB, dL, aF, g, bz, q, ad, bA); #c1(KDM4C) c2(NP_001140167) c3(lysine-specific demethylase 4C isoform 3) c4( ) c5(60025, 60026, 60027, 60028, 60029) c6( ) c7(Gl, T, oe, ht, aw, eX, jh, hy, fr, cd, ad, aT, eV, Gw, ae, jf, ff, en, FX, fq, bN, Gu, it); #c1(KDM4C) c2(NP_001140168) c3(lysine-specific demethylase 4C isoform 4) c4(60030, 60031) c5(60032, 60033, 60034, 60035, 60036, 60037, 60038) c6( ) c7(Gl, T, oe, ht, aw, eX, jh, hy, fr, cd, ad, aT, eV, Gw, ae, jf, ff, en, FX, fq, bN, Gu, it); #c1(KDM4C) c2(NP_001291269) c3(lysine-specific demethylase 4C isoform 6) c4(115626) c5(115627, 115628, 115629, 115630, 115631, 115632) c6( ) c7(Gl, T, oe, ht, aw, eX, jh, hy, fr, cd, ad, aT, eV, Gw, ae, jf, ff, en, FX, fq, bN, Gu, it); #c1(KDM4C) c2(NP_055876) c3(lysine-specific demethylase 4C isoform 1) c4(184092, 184093, 184094) c5(184095, 184096, 184097, 184098, 184099, 184100, 184101, 184102, 184103, 184104, 184105, 184106, 184107, 184108, 184109, 184110, 184111, 184112, 184113) c6( ) c7(Gl, T, oe, ht, aw, eX, jh, hy, fr, cd, ad, aT, eV, Gw, ae, jf, f, en, FX, fq, bN, Gu, it); #c1(KDM4C) c2(XP_011516111) c3(lysine-specific demethylase 4C isoform X3) c4( ) c5(309333) c6( ) c7(Gl, T, oe, ht, aw, eX, jh, hy, fr, cd, ad, aT, eV, Gw, ae, jf, ff, en, FX, fq, bN, Gu, it); #c1(KDM4C) c2(XP_011516113) c3(lysine-specific demethylase 4C isoform X4) c4(309334) c5(309335, 309336, 309337, 309338, 309339) c6( ) c7(Gl, T, oe, ht, aw, eX, jh, hy, fr, cd, ad, aT, eV, Gw, ae, jf, ff, en, FX, fq, bN, Gu, it); #c1(KDM4C) c2(XP_011516118) c3(lysine-specific demethylase 4C isoform X8) c4(309340) c5(309341, 309342, 309343, 309344, 309345, 309346) c6( ) c7(Gl, T, oe, ht, aw, eX, jh, hy, fr, cd, ad, aT, eV, Gw, ae, jf, ff, en, FX, fq, bN, Gu, it); #c1(KDM5A) c2(NP_001036068) c3(lysine-specific demethylase 5A) c4(33920, 33921, 33922, 33923) c5(33924, 33925, 33926, 33927, 33928, 33929, 33930, 33931, 33932, 33933, 33934, 33935, 33936, 33937, 33938, 33939, 33940, 33941, 33942, 33943, 33944, 33945, 33946, 33947, 33948) c6( ) c7(ck, yi, mR, Dy, eM, bP, aW, aT, aA, n, Hn, aF, nA, hF, bt, bz, B); #c1(KDM5B) c2(NP_006609) c3(lysine-specific demethylase 5B) c4(166602) c5(166603, 166604, 166605, 166606, 166607, 166608, 166609, 166610, 166611, 166612, 166613, 166614, 166615, 166616, 166617, 166618, 166619, 166620, 166621, 166622) c6( ) c7(gx, T, kP, aW, Y, oV, yk, eL, r, aT, cd, cz, dL, Hn, bu, bz, q, ad); #c1(KDM5B) c2(XP_011507391) c3(lysine-specific demethylase 58 isoform X3) c4(297250) c5( ) c6( ) c7(gx, T, kP, aW, Y, oV, yk, eL, r, aT, cd, cz, dL, Hn, bu, bz, q, ad); #c1(KDM5B) c2(XP_011507393) c3(lysine-specific demethylase 5B isoform X4) c4( ) c5(297251, 297252, 297253) c6( ) c7(gx, T, kP, aW, Y, oV, yk, eL, r, aT, cd, cz, dL, Hn, bu, bz, q, ad); #c1(KDM5B) c2(XP_011507394) c3(lysine-specific demethylase 5B isoform X5) c4(297254, 297255) c5(297256, 297257, 297258, 297259, 297260) c6( ) c7(gx, T, kP, aW, Y, oV, yk, eL, r, aT, cd, cz, dL, Hn, bu, bz, q, ad); #c1(KDM5C) c2(NP_001140174) c3(lysine-specific demethylase 5C isoform 2) c4(60039) c5(60040, 60041, 60042, 60043, 60044, 60045, 60046) c6( ) c7(py, Rq, bVs, ha, nf, fr, nA, lu, bN); #c1(KDM5C) c2(NP_001269551) c3(lysine-specific demethylase 5C isoform 3) c4(101901) c5(101902, 101903, 101904, 101905, 101906, 101907) c6( ) c7(py, Rq, bVs, ha, nf, fr, nA, lu, bN); #c1(KDM5C) c2(NP_004178) c3(lysine-specific demethylase 5C isoform 1) c4(144111) c5(144112, 144113, 144114, 144115, 144116, 144117, 144118) c6( ) c7(py, Rq, bVs, ha, nf, fr, nA, lu, bN); #c1(KDM5C) c2(XP_005262092) c3(lysine-specific demethylase 5C isoform X1) c4(277744) c5(277745, 277746, 277747, 277748, 277749, 277750) c6( ) c7(py, Rq, bVs, ha, nf, fr, nA, lu, bN); #c1(KDM5C) c2(XP_011529128) c3(lysine-specific demethylase 5C isoform X4) c4(324451) c5(324452, 324453, 324454, 324455, 324456, 324457) c6( ) c7(py, Rq, bVs, ha, nf, fr, nA, lu, bN); #c1(KDM5D) c2(NP_001140177) c3(lysine-specific demethylase 5D isoform 1) c4(60047) c5(60048, 60049, 60050, 60051, 60052, 60053) c6( ) c7(AV, aF, fr, aEE, aA); #c1(KDM5D) c2(NP_001140178) c3(lysine-specific demethylase 5D isoform 3) c4(60054) c5(60055, 60056, 60057, 60058, 60059, 60060) c6( ) c7(AV, aF, fr, aEE, aA); #c1(KDM5D) c2(NP_004644) c3(lysine-specific demethylase 5D isoform 2) c4(148473, 148474) c5(148475, 148476, 148477, 148478, 148479, 148480, 148481, 148482, 148483, 148484, 148485, 148486, 148487) c6( ) c7(AV, aF, fr, aEE, aA); #c1(KDM5D) c2(XP_005262617) c3(lysine-specific demethylase 5D isoform X2) c4(278013) c5(278014, 278015, 278016, 278017, 278018, 278019) c6( ) c7(AV, aF, fr, aEE, aA); #c1(KDM5D) c2(XP_005262619) c3(lysine-specific demethylase 5D isoform X4) c4(278020) c5(278021, 278022, 278023, 278024, 278025, 278026) c6( ) c7(AV, aF, fr, aEE, aA); #c1(KDM5D) c2(XP_011529770) c3(lysine-specific demethylase 50 isoform X1) c4(325434) c5(325435, 325436, 325437, 325438, 325439, 325440) c6( ) c7(AV, aF, fr, aEE, aA); #c1(KDM6A) c2(NP_001278344) c3(lysine-specific demethylase 6A isoform 1) c4(109557, 109558) c5(109559) c6( ) c7(T, id, fd, n, fr, nA, lu, XW, py, bVt, qK, aT, hD, dL, jj, eL, jf, aW, bUh, bw, ck, it); #c1(KDM6A) c2(NP_001278345) c3(lysine-specific demethylase 6A isoform 2) c4(109560) c5(109561, 109562, 109563, 109564, 109565, 109566) c6( ) c7(T, id, fd, n, fr, nA, lu, XW, py, bVt, qK, aT, hD, dL, jj, eL, jf, aW, bUh, bw, ck, it); #c1(KDM6A) c2(NP_001278346) c3(lysine-specific demethylase 6A isoform 4) c4(109567, 109568) c5(109569, 109570, 109571, 109572, 109573, 109574) c6( ) c7(T, id, fd, n, fr, nA, lu, XW, py, bVt, qK, aT, hD, dL, jj, eL, jf, aW, bUh, bw, ck, it); #c1(KDM6A) c2(NP_001278347) c3(lysine-specific demethylase 6A isoform 5) c4(109575) c5(109576, 109577, 109578, 109579, 109580, 109581, 109582) c6( ) c7(T, id, fd, n, fr, nA, lu, XW, py, bVt, qK, aT, hD, dL, jj, eL, jf, aW, bUh, bw, ck, it); #c1(KDM6A) c2(NP_001278350) c3(lysine-specific demethylase 6A isoform 6) c4( ) c5(109590) c6( ) c7(T, id, fd, n, fr, nA, lu, XW, py, bVt, qK, aT, hD, dL, jj, eL, jf, aW, bUh, bw, ck, it); #c1(KDM6A) c2(NP_066963) c3(lysine-specific demethylase GA isoform 3) c4(207840) c5(207841, 207842, 207843, 207844, 207845, 207846) c6( ) c7(T, id, fd, n, fr, nA, lu, XW, py, bVt, qK, aT, hD, dL, jj, eL, jf, aW, bUh, bw, ck, it); #c1(KDM6A) c2(XP_005272716) c3(lysine-specific demethylase 6A isoform X19) c4(284161) c5(284162, 284163, 284164, 284165, 284166, 284167) c6( ) c7(T, id, fd, n, fr, nA, lu, XW, py, bVt, qK, aT, hD, dL, jj, eL, jf, aW, bUh, bw, ck, it); #c1(KDM6A) c2(XP_011542262) c3(lysine-specific demethylase 6A isoform X4) c4( ) c5(340845, 340846, 340847, 340848, 340849, 340850, 340851, 340852, 340853, 340854, 340855) c6( ) c7(T, id, fd, n, fr, nA, lu, XW, py, bVt, qK, aT, hD, dL, jj, eL, jf, aW, bUh, bw, ck, it); #c1(KDM6A) c2(XP_011542275) c3(lysine-specific demethylase 6A isoform X18) c4( ) c5(340856, 340857, 340858, 340859, 340860, 340861) c6( ) c7(T, id, fd, n, fr, nA, lu, XW, py, bVt, qK, aT, hD, dL, jj, eL, jf, aW, bUh, bw, ck, it); #c1(KDM6A) c2(XP_011542276) c3(lysine-specific demethylase 6A isoform X20) c4(340862, 340863) c5(340864, 340865, 340866, 340867, 340868, 340869, 340870, 340871, 340872, 340873) c6( ) c7(T, id, fd, n, fr, nA, lu, XW, py, bVt, qK, aT, hD, dL, jj, eL, jf, aW, bUh, bw, ck, it); #c1(KDM6A) c2(XP_011542277) c3(lysine-specific demethylase 6A isoform X21) c4(340874) c5(340875, 340876, 340877, 340878, 340879, 340880) c6( ) c7(T, id, fd, n, fr, nA, lu, XW, py, bVt, qK, aT, hD, dL, jj, eL, jf, aW, bUh, bw, ck, it); #c1(KDM6B) c2(NP_001073893) c3(lysine-specific demethylase 6B) c4(36337) c5(36338, 36339, 36340, 36341, 36342, 36343) c6( ) c7(T, B, bE, qK, eZ, bB, aq, fr, br, nA, bw, ae, X, ad, bA); #c1(KDM6B) c2(XP_005256611) c3(lysine-specific demethylase 6B isoform X2) c4(274224) c5(274225, 274226, 274227, 274228, 274229, 274230) c6( ) c7(T, B, bE, qK, eZ, bB, aq, fr, br, nA, bw, ae, X, ad, bA); #c1(KDM6B) c2(XP_011522056) c3(lysine-specific demethylase GB isoform X4) c4( ) c5(316716) c6( ) c7(T, B, bE, qK, eZ, bB, aq, fr, br, nA, bw, ae, X, ad, bA); #c1(KDR) c2(NP_002244) c3(vascular endothelial growth factor receptor 2 precursor) c4(126868) c5(126869, 126870, 126871, 126872, 126873, 126874) c6( ) c7(bM, T, aC, UH, pf, bVx, ea, if, al, bO, eb, bB, fd, gf, ci, Hr, x, Vp, bv, aGm, aE, il, fm, rw, M, dV, Vv, K, cp, li, aW, fc, hl, nY, g, as, cm, qJ, y, eM, nB, iT, mx, vy, cj, uJ, k, m, Xu, Vj, dD, sw, ht, NT, n, BH, aj, fr, ct, bd, rP, zP, eh, ai, bis, b, ft, jz, i, h, lG, Jn, Um, dx, cY, hi, bkN, mR, MD, aJ, hQ, aq, cM, fs, W, bP, dB, Vy, hL, auh, bVv, hR, at, aAl, B, bL, fM, dA, eR, jp, aA, cz, WD, zN, hy, j, wo, il, q, J, eA, eJ, nv, BJ, MP, aT, cy, Tj, bq, rv, ae, af, dl, eu, jf, iF, PK, iU, vT, nO, oV, Y, r, en, bu, cl, t, Vi, acp, aoy, pb, ck, ov, beH, beJ, kP, c, ac, nM, wO, nG, JL, dy, EY, bt, hS, gx, id, ow, qz, iW, oA, jh, dG, ah, eq, hF, gb, ad, afb, Jm, fU, dP, F, ard, ib, py, yt, oz, Hk, ji, fb, nJ, uQ, d, ue, cH, cW, ff, cr, eZ, aF, iV, bVw, bz, Hv, ek, Hp, es, nb, bE, bVu, ajw, aS, oQ, GF, lf, vt, jr); #c1(KDSR) c2(NP_002026) c3(3-ketodihydrosphingosine reductase precursor) c4(123413) c5(123414, 123415, 123416, 123417, 123418, 123419, 123420, 123421, 123422, 123423, 123424, 123425, 123426, 123427, 123428, 123429) c6(123430, 123431, 123432, 123433) c7(kf, T, fM, j); #c1(KDSR) c2(XP_006722496) c3(3-ketodihydrosphingosine reductase isoform X1) c4(294720) c5( ) c6( ) c7(kf, T, fM, j); #c1(KEAP1) c2(NP_036421) c3(kelch-like ECH-associated protein 1) c4(173686) c5(173687, 173688, 173689, 173690, 173691, 173692, 173693, 173694, 173695, 173696, 173697) c6( ) c7(T, eR, e, aWG, bB, aj, fr, ci, C, aY, q, J, er, fU, av, cc, aE, aT, io, lG, K, hB, an, af, Zm, aa, s, aW, ff, r, Qq, nY, en, g, t, iK, bz, nM, c, eM, bE, Qu, dh, U, at, aS, m, lC, bt, it, B); #c1(KEL) c2(NP_000411) c3(kell blood group glycoprotein) c4(8654) c5(8655, 8656, 8657, 8658, 8659, 8660) c6( ) c7(bL, bM, ZM, aw, qd, eB, dV, ZU, lf, bgl); #c1(KEL) c2(XP_005277046) c3(kell blood group glycoprotein isoform X2) c4( ) c5(285749) c6( ) c7(bL, bM, ZM, aw, qd, eB, dV, ZU, lf, bgl); #c1(KERA) c2(XP_011536083) c3(keratocan isoform X1) c4(333419) c5(333420, 333421, 333422, 333423, 333424, 333425) c6( ) c7(dD, pN, bmV, ea, bVy, ab, eq, bVz); #c1(KHDC3L) c2(NP_001017361) c3(KHDC3-like protein) c4(26252) c5(26253, 26254, 26255, 26256, 26257, 26258) c6( ) c7(CN, jv, ZH); #c1(KHDRBS2) c2(NP_689901) c3(KH domain-containing, RNA-binding, signal transduction-associated protein 2) c4(244325) c5(244326, 244327, 244328, 244329, 244330, 244331) c6( ) c7(Y, iu, K); #c1(KHDRBS2) c2(XP_006715417) c3(KH domain-containing, RNA-binding, signal transduction-associated protein 2 isoform X1) c4(289524) c5(289525, 289526, 289527, 289528, 289529, 289530) c6( ) c7(Y, iu, K); #c1(KHDRBS3) c2(NP_006549) c3(KH domain-containing, RNA-binding, signal transduction-associated protein 3) c4(166163) c5(166164, 166165, 166166, 166167, 166168, 166169) c6( ) c7(jf, aW, ha, aT, n, K, bVA, X); #c1(KHK) c2(NP_000212) c3(ketohexokinase isoform a) c4(4628) c5(4629, 4630, 4631, 4632, 4633, 4634) c6( ) c7(es, T, bVB, go, bVC, fr, bq); #c1(KHK) c2(NP_006479) c3(ketohexokinase isoform b) c4(165576) c5(165577, 165578, 165579, 165580, 165581, 165582) c6( ) c7(es, T, bVB, go, bVC, fr, bq); #c1(KHSRP) c2(NP_003676) c3(far upstream element-binding protein 2) c4(140116, 140117) c5( ) c6( ) c7(bO, aA, aj, hF, eA, aR, amW, ft, ec, aCJ, aql, aT, kf, dz, K, hS, zy, H, WZ, jf, eG, LC, Y, Ls, aF, hf, eE, cm, i, c, iT, Fi, bt, it, B, L); #c1(KHSRP) c2(XP_011526697) c3(far upstream element-binding protein 2 isoform X2) c4(320996) c5(320997, 320998, 320999, 321000, 321001, 321002, 321003, 321004, 321005, 321006, 321007, 321008, 321009, 321010, 321011, 321012, 321013, 321014) c6( ) c7(bO, aA, aj, hF, eA, aR, amW, ft, ec, aCJ, aql, aT, kf, dz, K, hS, zy, H, WZ, jf, eG, LC, Y, Ls, aF, hf, eE, cm, li, c, iT, Fi, bt, it, B, L); #c1(KIAA0020) c2(NP_055693) c3(pumilio domain-containing protein KIAA0020) c4(182804) c5(182805, 182806, 182807, 182808, 182809, 182810) c6( ) c7(hp); #c1(KIAA0040) c2(NP_055471) c3(uncharacterized protein KIAA0040) c4(181339) c5(181340, 181341, 181342, 181343, 181344, 181345, 181346, 181347) c6( ) c7(cd); #c1(KIAA0040) c2(XP_011508485) c3(uncharacterized protein KIAA0040 isoform X2) c4(299201) c5(299202, 299203, 299204, 299205, 299206, 299207, 299208, 299209, 299210) c6( ) c7(cd); #c1(KIAA0100) c2(NP_055495) c3(UPF0378 protein KIAA0100 precursor) c4(181505, 181506, 181507, 181508) c5(181509, 181510, 181511, 181512, 181513, 181514, 181515, 181516, 181517, 181518, 181519, 181520, 181521, 181522, 181523, 181524, 181525, 181526, 181527, 181528, 181529, 181530, 181531, 181532, 181533, 181534, 181535, 181536, 181537, 181538, 181539, 181540) c6( ) c7(aW, ci, kP, A, aT); #c1(KIAA0100) c2(XP_006722261) c3(UPF0378 protein KIAA0100 isoform X2) c4( ) c5(294671, 294672, 294673) c6( ) c7(aW, ci, kP, AD, aT); #c1(KIAA0101) c2(NP_001025160) c3(PCNA-associated factor isoform 2) c4(29367) c5(29368, 29369, 29370, 29371, 29372, 29373) c6( ) c7(gx, T, dD, ea, ht, aA, bB, j, ci, eq, py, aE, aT, iv, ey, K, lQ, hu, ae, qt, aW, Y, iw, cr, aq, aF, Hu, g, iR, LV, cm, bz, DF, es, atU, eM, nB, bE, A, JM, Th, B); #c1(KIAA0101) c2(NP_055551) c3(PCNA-associated factor isoform 1) c4(181896) c5(181897, 181898, 181899, 181900, 181901, 181902) c6( ) c7(gx, T, dD, ea, ht, aA, bB, j, ci, eq, py, aE, aT, iv, ey, K, lQ, hu, ae, qt, aW, Y, iw, cr, aq, aF, Hu, g, iR, LV, cm, bz, DF, es, atU, eM, nB, bE, iA, JM, Th, B); #c1(KIAA0196) c2(NP_055661) c3(WASH complex subunit strumpellin) c4(182591) c5(182592, 182593, 182594, 182595, 182596, 182597) c6( ) c7(bb, aV, oj, ajb, cz, bVD, ci, bi, cm, bVE, ae, ad); #c1(KIAA0226) c2(NP_001139114) c3(run domain Beclin-1 interacting and cysteine-rich containing protein isoform 1) c4(58699) c5(58700, 58701, 58702, 58703, 58704) c6( ) c7(bVF); #c1(KIAA0226) c2(NP_055502) c3(run domain Beclin-I interacting and cysteine-rich containing protein isoform 2) c4(181589) c5(181590, 181591, 181592, 181593, 181594, 181595) c6( ) c7(bVF); #c1(KIAA0226) c2(XP_005269431) c3(run domain Beclin-1 interacting and cysteine-rich containing protein isoform X3) c4( ) c5(282564) c6( ) c7(bVF); #c1(KIAA0226) c2(XP_006713891) c3(run domain Beclin-1 interacting and cysteine-rich containing protein isoform X2) c4(287414) c5(287415, 287416, 287417, 287418, 287419, 287420) c6( ) c7(bVF); #c1(KIAA0226L) c2(NP_001273691) c3(uncharacterized protein KIAA0226-like isoform b) c4(105623) c5(105624, 105625, 105626, 105627, 105628, 105629, 105630, 105631, 105632, 105633, 105634, 105635, 105636, 105637, 105638, 105639, 105640, 105641, 105642, 105643, 105644) c6( ) c7(c, if, ff, ht, ey, en, qN); #c1(KIAA0226L) c2(NP_001273692) c3(uncharacterized protein KIAA0226-like isoform c) c4(105645) c5(105646, 105647, 105648, 105649, 105650, 105651) c6( ) c7(c, if, ff, ht, ey, en, qN); #c1(KIAA0226L) c2(NP_001273693) c3(uncharacterized protein KIAA0226-like isoform d) c4( ) c5(105652, 105653, 105654, 105655, 105656) c6( ) c7(c, if, ff, ht, ey, en, qN); #c1(KIAA0226L) c2(NP_001273694) c3(uncharacterized protein KIAA0226-like isoform e) c4(105657) c5(105658, 105659, 105660, 105661, 105662, 105663) c6( ) c7(c, if, ff, ht, ey, en, qN); #c1(KIAA0226L) c2(NP_079389) c3(uncharacterized protein KIAA0226-like isoform a) c4(217869, 217870, 217871, 217872) c5(217873, 217874, 217875, 217876, 217877, 217878, 217879, 217880, 217881, 217882, 217883, 217884) c6( ) c7(c, if, ff, ht, ey, en, qN); #c1(KIAA0232) c2(NP_001094060) c3(uncharacterized protein KIAA0232) c4(40560) c5(40561, 40562, 40563, 40564, 40565, 40566, 40567, 40568, 40569, 40570) c6( ) c7(j); #c1(KIAA0232) c2(XP_006713998) c3(uncharacterized protein KIAA0232 isoform X2) c4( ) c5(287489, 287490, 287491, 287492, 287493, 287494) c6( ) c7(j); #c1(KIAA0232) c2(XP_011511899) c3(uncharacterized protein KIAA0232 isoform X1) c4(302604) c5(302605) c6( ) c7(j); #c1(KIAA0319) c2(NP_001161846) c3(dyslexia-associated protein KIAA0319 isoform b) c4(66150) c5(66151, 66152, 66153, 66154, 66155, 66156) c6( ) c7(T, hl, qx, ha, VT, zN, ti, fu, nA, alP, afz, aR, xt, ad, yn, Ae, aT, bK, mL, bU, he, afx, bVH, Wq, ae, bVG, al, bVl, Lg, bdG, ago, PK, afy, bPl, aW, FD, gl, dq, lo, Fy, Ku, cm, bVJ, dr, bVK, kn, bN, bdT, alC, WO, afZ, FB, U, bdU, yA, bC, UF, Wl, xg, lf); #c1(KIAA0319) c2(NP_001161849) c3(dyslexia-associated protein KIAA0319 isoform d precursor) c4(66157) c5(66158, 66159, 66160, 66161, 66162, 66163) c6( ) c7(T, hl, qx, ha, VT, zN, ti, fu, nA, alP, afz, aR, xt, ad, yn, Ae, aT, bK, mL, bU, he, afx, bVH, Wq, ae, bVG, al, bVl, Lg, bdG, ago, PK, afy, bPl, aW, FD, gl, dq, lo, Fy, Ku, cm, bVJ, dr, bVK, kn, bN, bdT, alC, WO, afZ, FB, U, bdU, yA, bC, UF, Wl, xg, lf); #c1(KIAA0319) c2(NP_055624) c3(dyslexia-associated protein KIAA0319 isoform a precursor) c4(182330) c5(182331, 182332, 182333, 182334, 182335, 182336, 182337, 182338, 182339, 182340, 182341, 182342, 182343, 182344, 182345) c6( ) c7(T, hl, qx, ha, VT, zN, ti, fu, nA, alP, afz, aR, xt, ad, yn, Ae, aT, bK, mL, bU, he, afx, bVH, Wq, ae, bVG, al, bVl, Lg, bdG, ago, PK, afy, bPl, aW, FD, gl, dq, lo, Fy, Ku, cm, bVJ, dr, bVK, kn, bN, bdT, alC, WO, afZ, FB, U, bdU, yA, bC, UF, W, xg, lf); #c1(KIAA0319) c2(XP_011513326) c3(dyslexia-associated protein KIAA0319 isoform X3) c4(305588) c5(305589, 305590) c6( ) c7(T, hl, qx, ha, VT, zN, ti, fu, nA, alP, afz, aR, xt, ad, yn, Ae, aT, bK, mL, bU, he, afx, bVH, Wq, ae, bVG, al, bVl, Lg, bdG, ago, PK, afy, bPl, aW, FD, gl, dq, lo, Fy, Ku, cm, bVJ, dr, bVK, kn, bN, bdT, alC, WO, afZ, FB, U, bdU, yA, bC, UF, Wl, xg, lf); #c1(KIAA0319L) c2(XP_011540484) c3(dyslexia-associated protein KIAA0319-like protein isoform X3) c4(338958, 338959, 338960, 338961) c5(338962, 338963, 338964, 338965, 338966, 338967, 338968, 338969, 338970, 338971, 338972, 338973, 338974, 338975, 338976, 338977, 338978, 338979) c6( ) c7(eR, pb, yA, eZ); #c1(KIAA0355) c2(NP_055501) c3(uncharacterized protein KIAA0355) c4(181574, 181575) c5(181576, 181577, 181578, 181579, 181580, 181581, 181582, 181583, 181584, 181585, 181586, 181587, 181588) c6( ) c7(bK); #c1(KIAA0391) c2(NP_001243607) c3(mitochondrial ribonuclease P protein 3 isoform 2) c4(91175, 91176, 91177, 91178) c5(91179, 91180, 91181, 91182, 91183, 91184, 91185, 91186, 91187, 91188, 91189, 91190, 91191) c6( ) c7(cL, aw, j, cr); #c1(KIAA0391) c2(NP_001243608) c3(mitochondrial ribonuclease P protein 3 isoform 3) c4( ) c5(91192, 91193, 91194, 91195, 91196, 91197, 91198, 91199, 91200) c6( ) c7(cL, aw, j, cr); #c1(KIAA0391) c2(NP_001243610) c3(mitochondrial ribonuclease P protein 3 isoform 4) c4( ) c5(91201, 91202, 91203, 91204, 91205) c6( ) c7(cL, aw, j, cr); #c1(KIAA0391) c2(NP_055487) c3(mitochondrial ribonuclease P protein 3 isoform 1 precursor) c4(181441, 181442, 181443) c5(181444, 181445, 181446, 181447, 181448, 181449, 181450, 181451, 181452, 181453, 181454, 181455, 181456, 181457, 181458, 181459, 181460, 181461, 181462, 181463, 181464, 181465, 181466, 181467, 181468, 181469, 181470) c6( ) c7(cL, aw, j, cr); #c1(KIAA0586) c2(NP_001231118) c3(protein TALPID3 isoform 1) c4(87205) c5(87206, 87207, 87208, 87209, 87210, 87211) c6( ) c7(zG, Az, c); #c1(KIAA0586) c2(NP_001231119) c3(protein TALPID3 isoform 2) c4(87212) c5(87213, 87214, 87215, 87216, 87217, 87218, 87219, 87220, 87221, 87222, 87223) c6( ) c7(zG, Az, c); #c1(KIAA0586) c2(NP_001231120) c3(protein TALPID3 isoform 3) c4(87224) c5( ) c6( ) c7(zG, Az, c); #c1(KIAA0586) c2(NP_001231121) c3(protein TALPID3 isoform 4) c4(87225) c5(87226, 87227, 87228, 87229, 87230, 87231, 87232) c6( ) c7(zG, Az, c); #c1(KIAA0586) c2(NP_001231122) c3(protein TALPID3 isoform 6) c4( ) c5(87233) c6( ) c7(zG, Az, c); #c1(KIAA0586) c2(NP_055564) c3(protein TALPID3 isoform 5) c4(181954) c5(181955, 181956, 181957, 181958, 181959, 181960) c6( ) c7(zG, Az, c); #c1(KIAA0825) c2(NP_001139150) c3(uncharacterized protein KIAA0825 isoform 1) c4(58860) c5(58861, 58862, 58863, 58864, 58865, 58866, 58867, 58868, 58869, 58870) c6( ) c7(j, hB); #c1(KIAA0825) c2(NP_775936) c3(uncharacterized protein KIAA0825 isoform 2) c4( ) c5(250756, 250757, 250758, 250759, 250760, 250761) c6( ) c7(j, hB); #c1(KIAA0825) c2(XP_011541633) c3(uncharacterized protein KIAA0825 isoform X4) c4(340178, 340179) c5( ) c6( ) c7(j, hB); #c1(KIAA0825) c2(XP_011541636) c3(uncharacterized protein KIAA0825 isoform X7) c4( ) c5(340180, 340181, 340182, 340183, 340184, 340185) c6( ) c7(j, hB); #c1(KIAA0907) c2(NP_055764) c3(UPF0469 protein KIAA0907) c4(183343, 183344, 183345) c5(183346, 183347, 183348, 183349, 183350, 183351, 183352, 183353, 183354, 183355, 183356, 183357, 183358, 183359, 183360, 183361, 183362, 183363, 183364) c6( ) c7(bz); #c1(KIAA0922) c2(NP_001124479) c3(transmembrane protein 131-like isoform 1 precursor) c4(49132) c5(49133, 49134, 49135, 49136, 49137, 49138) c6( ) c7(aw); #c1(KIAA0922) c2(NP_056011) c3(transmembrane protein 131-like isoform 2 precursor) c4(185119) c5(185120, 185121, 185122, 185123, 185124, 185125) c6( ) c7(aw); #c1(KIAA0922) c2(XP_011530085) c3(transmembrane protein 131-like isoform X4) c4(325913) c5(325914, 325915, 325916, 325917, 325918, 325919) c6( ) c7(aw); #c1(KIAA1024) c2(NP_056021) c3(UPF0258 protein KIAA1024) c4(185200) c5(185201, 185202, 185203, 185204, 185205, 185206) c6( ) c7(De); #c1(KIAA1033) c2(NP_056090) c3(WASH complex subunit 7 isoform 2) c4(185642) c5(185643, 185644, 185645, 185646, 185647, 185648) c6( ) c7(bK, nA); #c1(KIAA1109) c2(XP_005263344) c3(uncharacterized protein KIAA1109 isoform X1) c4(278444) c5(278445, 278446, 278447, 278448, 278449, 278450, 278451, 278452, 278453, 278454) c6( ) c7(Ki, A, Kk, ge, nf, hp, Ku, iV, y); #c1(KIAA1109) c2(XP_006714406) c3(uncharacterized protein KIAA1109 isoform X14) c4(287830) c5(287831, 287832, 287833, 287834, 287835, 287836) c6( ) c7(Ki, A, Kk, ge, nf, hp, Ku, iV, y); #c1(KIAA1109) c2(XP_006714407) c3(uncharacterized protein KIAA1109 isoform X15) c4( ) c5(287837, 287838, 287839, 287840, 287841, 287842) c6( ) c7(Ki, A, Kk, ge, nf, hp, Ku, iV, y); #c1(KIAA1109) c2(XP_011530632) c3(uncharacterized protein KIAA1109 isoform X12) c4(326727) c5( ) c6( ) c7(Ki, A, Kk, ge, nf, hp, Ku, iV, y); #c1(KIAA1147) c2(NP_001073861) c3(protein LCHN) c4(36153) c5(36154, 36155, 36156, 36157, 36158, 36159, 36160) c6( ) c7(cy, vs, ek); #c1(KIAA1161) c2(NP_065753) c3(uncharacterized family 31 glucosidase KIAA1161) c4(205190, 205191) c5(205192, 205193, 205194, 205195, 205196, 205197, 205198, 205199, 205200, 205201, 205202, 205203) c6( ) c7(af); #c1(KIAA1211) c2(NP_065773) c3(uncharacterized protein KIAA1211) c4(205309) c5(205310, 205311, 205312, 205313, 205314, 205315, 205316, 205317, 205318, 205319, 205320, 205321, 205322, 205323, 205324, 205325, 205326, 205327) c6( ) c7(ad); #c1(KIAA1211) c2(XP_005265813) c3(uncharacterized protein KIAA1211 isoform X7) c4(279740) c5( ) c6( ) c7(ad); #c1(KIAA1211) c2(XP_006714108) c3(uncharacterized protein KIAA1211 isoform X8) c4( ) c5(287572, 287573) c6( ) c7(ad); #c1(KIAA1211) c2(XP_011532700) c3(uncharacterized protein KIAA1211 isoform X4) c4(328732) c5( ) c6( ) c7(ad); #c1(KIAA1211L) c2(XP_005263981) c3(uncharacterized protein KIAA1211-like isoform X2) c4(278637) c5(278638, 278639, 278640, 278641, 278642, 278643) c6( ) c7(bK); #c1(KIAA1217) c2(NP_001091970) c3(sickle tail protein homolog isoform 2) c4(39089) c5(39090, 39091, 39092, 39093, 39094) c6( ) c7(aC, iM, je, rl, cd, Ek); #c1(KIAA1217) c2(NP_001269696) c3(sickle tail protein homolog isoform 4) c4(102581) c5(102582, 102583, 102584, 102585, 102586, 102587, 102588, 102589, 102590, 102591, 102592, 102593) c6( ) c7(aC, iM, je, rl, cd, Ek); #c1(KIAA1217) c2(NP_001269697) c3(sickle tail protein homolog isoform 5) c4(102594) c5(102595, 102596, 102597, 102598, 102599, 102600, 102601) c6( ) c7(aC, iM, je, rl, cd, Ek); #c1(KIAA1217) c2(NP_062536) c3(sickle tail protein homolog isoform 1) c4(201832) c5(201833, 201834, 201835, 201836, 201837, 201838) c6( ) c7(aC, iM, je, rl, cd, Ek); #c1(KIAA1217) c2(XP_005252564) c3(sickle tail protein homolog isoform X15) c4( ) c5(271878) c6( ) c7(aC, iM, je, rl, cd, Ek); #c1(KIAA1217) c2(XP_005252573) c3(sickle tail protein homolog isoform X21) c4( ) c5(271879, 271880, 271881, 271882, 271883, 271884, 271885, 271886, 271887, 271888) c6( ) c7(aC, iM, je, rl, cd, Ek); #c1(KIAA1217) c2(XP_011517859) c3(sickle tail protein homolog isoform X11) c4(311785, 311786) c5( ) c6( ) c7(aC, iM, je, rl, cd, Ek); #c1(KIAA1217) c2(XP_011517861) c3(sickle tail protein homolog isoform X14) c4(311787) c5(311788) c6( ) c7(aC, iM, je, rl, cd, Ek); #c1(KIAA1279) c2(NP_056449) c3(KIFI-binding protein) c4(187595) c5(187596, 187597, 187598, 187599, 187600, 187601) c6( ) c7(mO, ahl, nA, an, QK, ahE, bVL); #c1(KIAA1324) c2(XP_011540127) c3(UPF0577 protein KIAA1324 isoform X1) c4(338475, 338476) c5( ) c6( ) c7(auY, zY, zX, hL, Qm, m, bX, cl, bbr); #c1(KIAA1324) c2(XP_011540128) c3(UPF0577 protein KIAA1324 isoform X2) c4(338477) c5( ) c6( ) c7(auY, zY, zX, hL, Qm, m, bX, cl, bbr); #c1(KIAA1324) c2(XP_011540129) c3(UPF0577 protein KIAA1324 isoform X3) c4(338478) c5(338479, 338480, 338481, 338482, 338483, 338484) c6( ) c7(auY, zY, zX, hL, Qm, m, bX, cl, bbr); #c1(KIAA1324L) c2(NP_001136221) c3(UPF0577 protein KIAA1324-like isoform 1 precursor) c4(54521) c5(54522, 54523, 54524, 54525, 54526, 54527) c6( ) c7(fq); #c1(KIAA1324L) c2(NP_001278920) c3(UPF0577 protein KIAA1324-like isoform 4) c4(110991) c5(110992, 110993, 110994, 110995, 110996, 110997) c6( ) c7(fq); #c1(KIAA1324L) c2(NP_689961) c3(UPF0577 protein KIAA1324-like isoform 2) c4(244588) c5(244589, 244590, 244591, 244592, 244593, 244594) c6( ) c7(fq); #c1(KIAA1324L) c2(XP_006715958) c3(UPF0577 protein KIAA1324-like isoform X1) c4(289952) c5(289953, 289954, 289955, 289956, 289957) c6( ) c7(fq); #c1(KIAA1324L) c2(XP_011514220) c3(UPF0577 protein KIAA1324-like isoform X2) c4( ) c5(306823) c6( ) c7(fq); #c1(KIAA1377) c2(NP_065853) c3(uncharacterized protein KIAA1377) c4(205912, 205913) c5(205914, 205915, 205916, 205917, 205918, 205919, 205920, 205921, 205922, 205923, 205924, 205925) c6( ) c7(axl); #c1(KIAA1456) c2(XP_005273641) c3(probable tRNA methyltransferase 9-like protein isoform X3) c4(284749, 284750, 284751) c5(284752, 284753) c6( ) c7(j, q, r); #c1(KIAA1456) c2(XP_005273643) c3(probable tRNA methyltransferase 9-like protein isoform X1) c4( ) c5(284754, 284755, 284756, 284757, 284758, 284759, 284760, 284761, 284762, 284763, 284764) c6( ) c7(j, q, r); #c1(KIAA1456) c2(XP_011542899) c3(probable tRNA methyltransferase 9-like protein isoform X2) c4(341495) c5(341496, 341497, 341498, 341499, 341500) c6( ) c7(j, q, r); #c1(KIAA1456) c2(XP_011542903) c3(probable tRNA methyltransferase 9-like protein isoform X4) c4( ) c5(341501) c6( ) c7(j, q, r); #c1(KIAA1456) c2(XP_011542904) c3(probable tRNA methyltransferase 9-like protein isoform X7) c4( ) c5(341502, 341503, 341504, 341505, 341506) c6( ) c7(j, q, r); #c1(KIAA1462) c2(XP_011517910) c3(junctional protein associated with coronary artery disease isoform X1) c4(311853) c5(311854, 311855, 311856, 311857, 311858, 311859, 311860) c6( ) c7(fK, e, j, qM, af); #c1(KIAA1462) c2(XP_011517912) c3(junctional protein associated with coronary artery disease isoform X2) c4( ) c5(311861) c6( ) c7(fK, e, j, qM, af); #c1(KIAA1468) c2(NP_065905) c3(lisH domain and HEAT repeat-containing protein KIAA1468) c4(206150) c5(206151, 206152, 206153, 206154, 206155, 206156) c6( ) c7(cd); #c1(KIAA1468) c2(XP_005266787) c3(lisH domain and HEAT repeat-containing protein KIAA1468 isoform X3) c4(280324) c5(280325, 280326, 280327, 280328, 280329, 280330) c6( ) c7(cd); #c1(KIAA1524) c2(NP_065941) c3(protein CIP2A) c4(206351, 206352) c5(206353, 206354, 206355, 206356, 206357, 206358, 206359) c6( ) c7(T, dD, hl, QO, ea, if, aA, bB, n, aj, pl, eq, ci, qN, fr, q, av, iu, ds, aT, iU, NE, Jl, aW, Y, eH, r, aF, cl, eF, t, cm, bz, ik, ck, kP, Dy, asl, bE, eM, nw, m, nz, bt, B, y, de); #c1(KIAA1524) c2(XP_011511358) c3(protein CIP2A isoform X2) c4( ) c5(301927, 301928, 301929, 301930, 301931) c6( ) c7(T, dD, hl, QO, ea, if, aA, bB, n, aj, pl, eq, ci, qN, fr, q, av, iu, ds, aT, iU, NE, Jl, aW, Y, eH, r, aF, cl, eF, t, cm, bz, ik, ck, kP, Dy, asl, bE, eM, nw, m, nz, bt, B, y, de); #c1(KIAA1549) c2(NP_001158137) c3(UPF0606 protein KIAA1549 isoform 2) c4(63176) c5(63177, 63178, 63179, 63180, 63181, 63182) c6( ) c7(auJ, Ew, ac, EQ, aun, Vl, as, Fc, anu, eA, Hp); #c1(KIAA1549) c2(NP_065961) c3(UPF0606 protein KIAA1549 isoform 1) c4(206492, 206493) c5(206494, 206495, 206496, 206497, 206498, 206499, 206500, 206501, 206502, 206503, 206504, 206505) c6( ) c7(auJ, Ew, ac, EQ, aun, Vl, as, Fc, anu, eA, Hp); #c1(KIAA1551) c2(XP_005253462) c3(uncharacterized protein KIAA1551 isoform X1) c4(272503, 272504, 272505) c5(272506, 272507, 272508, 272509, 272510, 272511, 272512, 272513, 272514, 272515, 272516) c6( ) c7(Y); #c1(KIAA1551) c2(XP_006719162) c3(uncharacterized protein KIAA1551 isoform X2) c4( ) c5(292231, 292232, 292233, 292234, 292235, 292236, 292237) c6( ) c7(Y); #c1(KIAA1598) c2(NP_001120683) c3(shootin-1 isoform a) c4(44540, 44541) c5(44542, 44543, 44544, 44545, 44546, 44547, 44548, 44549, 44550, 44551, 44552, 44553) c6( ) c7(cd, Nc, bvU); #c1(KIAA1598) c2(NP_001245228) c3(shootin-1 isoform d) c4( ) c5(92758) c6( ) c7(cd, Nc, bvU); #c1(KIAA1598) c2(NP_001245229) c3(shootin-1 isoform e) c4(92759) c5(92760, 92761, 92762, 92763, 92764) c6( ) c7(cd, Nc, bvU); #c1(KIAA1598) c2(NP_060800) c3(shootin-1 isoform b) c4(197898) c5(197899, 197900, 197901, 197902, 197903, 197904, 197905) c6( ) c7(cd, Nc, bvU); #c1(KIAA1715) c2(NP_001291937) c3(protein lunapark isoform 1 precursor) c4( ) c5(116535) c6( ) c7(aMb, an); #c1(KIAA1715) c2(NP_001291938) c3(protein lunapark isoform 2) c4(116536, 116537) c5(116538, 116539, 116540, 116541, 116542, 116543) c6( ) c7(aMb, an); #c1(KIAA1715) c2(NP_001291939) c3(protein lunapark isoform 3 precursor) c4(116544) c5(116545, 116546, 116547, 116548, 116549, 116550) c6( ) c7(aMb, an); #c1(KIAA1715) c2(NP_001291940) c3(protein lunapark isoform 5) c4( ) c5(116551, 116552, 116553, 116554, 116555) c6( ) c7(aMb, an); #c1(KIAA1715) c2(NP_085153) c3(protein lunapark isoform 4) c4(219488, 219489) c5(219490, 219491, 219492, 219493, 219494, 219495, 219496) c6( ) c7(aMb, an); #c1(KIAA1715) c2(XP_005246935) c3(protein lunapark isoform X2) c4( ) c5(267522, 267523, 267524, 267525, 267526) c6( ) c7(aMb, an); #c1(KIAA1715) c2(XP_005246936) c3(protein lunapark isoform X3) c4(267527) c5(267528, 267529, 267530, 267531, 267532, 267533) c6( ) c7(aMb, an); #c1(KIAA1804) c2(NP_115811) c3(mitogen-activated protein kinase kinase kinase MLK4) c4(224034, 224035, 224036) c5(224037, 224038, 224039, 224040, 224041, 224042, 224043, 224044, 224045, 224046, 224047, 224048) c6( ) c7(ac, r); #c1(KIAA1804) c2(XP_011542607) c3(mitogen-activated protein kinase kinase kinase MLK4 isoform X1) c4( ) c5(341174, 341175, 341176, 341177, 341178, 341179) c6( ) c7(ac, r); #c1(KIAA1841) c2(XP_005264663) c3(uncharacterized protein KIAA1841 isoform X1) c4(279153) c5(279154, 279155, 279156, 279157, 279158, 279159) c6( ) c7(hp); #c1(KIAA1841) c2(XP_011531429) c3(uncharacterized protein KIAA1841 isoform X2) c4(327564) c5(327565, 327566, 327567, 327568, 327569, 327570, 327571, 327572, 327573, 327574, 327575, 327576, 327577, 327578) c6( ) c7(hp); #c1(KIAA1919) c2(NP_699200) c3(sodium-dependent glucose transporter 1) c4(245891) c5(245892, 245893, 245894, 245895, 245896, 245897) c6( ) c7(A); #c1(KIAA2022) c2(NP_001008537) c3(protein KIAA2022) c4(23305, 23306) c5(23307, 23308, 23309, 23310, 23311, 23312, 23313, 23314, 23315, 23316, 23317, 23318, 23319, 23320, 23321, 23322, 23323, 23324) c6( ) c7(pH, bVM, Kr, bO, nf, abl, nA, Wl, rz); #c1(KIDINS220) c2(NP_065789) c3(kinase D-interacting substrate of 220 kDa) c4(205441, 205442, 205443, 205444, 205445) c5(205446, 205447, 205448, 205449, 205450, 205451, 205452, 205453, 205454, 205455, 205456, 205457, 205458, 205459, 205460, 205461, 205462, 205463, 205464, 205465, 205466, 205467, 205468, 205469, 205470, 205471, 205472, 205473, 205474, 205475, 205476, 205477, 205478, 205479) c6( ) c7(bM, T, jx, SW, hX, bv, ec, h, dL, K, eL, af, ajU, cp, Ar, oV, Y, ih, eZ, cm, nC, bz, ek, eM, U, pw, Xu, y); #c1(KIF11) c2(NP_004514) c3(kinesin-like protein KIF11) c4(147092) c5(147093, 147094, 147095, 147096, 147097, 147098) c6( ) c7(bL, ov, T, bVO, d, af, bVN, dB, aT, Vv, ey, PN, nA, QK, fK); #c1(KIF13A) c2(NP_001099036) c3(kinesin-like protein KIF13A isoform b) c4(41736, 41737) c5(41738, 41739, 41740, 41741, 41742, 41743, 41744, 41745, 41746, 41747, 41748, 41749) c6( ) c7(bK, ad); #c1(KIF13A) c2(NP_001099037) c3(kinesin-like protein KIF13A isoform c) c4(41750) c5(41751, 41752, 41753, 41754, 41755, 41756) c6( ) c7(bK, ad); #c1(KIF13A) c2(NP_001099038) c3(kinesin-like protein KIF13A isoform d) c4(41757) c5(41758, 41759, 41760, 41761, 41762, 41763) c6( ) c7(bK, ad); #c1(KIF13A) c2(NP_001230352) c3(kinesin-like protein KIF13A isoform e) c4(86244) c5(86245, 86246, 86247, 86248, 86249, 86250) c6( ) c7(bK, ad); #c1(KIF13A) c2(NP_071396) c3(kinesin-like protein KIF13A isoform a) c4(211422) c5(211423, 211424, 211425, 211426, 211427, 211428) c6( ) c7(bK, ad); #c1(KIF14) c2(NP_055690) c3(kinesin-like protein KIF14) c4(182790) c5(182791, 182792, 182793, 182794, 182795, 182796) c6( ) c7(T, aC, n, qN, AO, BA, av, aE, aT, ey, jf, B, aW, ff, en, g, t, bz, kP, eM, bP, m, iu); #c1(KIF15) c2(NP_064627) c3(kinesin-like protein KIF15) c4(203361, 203362) c5(203363, 203364, 203365, 203366, 203367, 203368, 203369, 203370, 203371, 203372, 203373, 203374) c6( ) c7(By, qy); #c1(KIF16B) c2(NP_001186795) c3(kinesin-like protein KIF1 GB isoform 1) c4(78599) c5(78600, 78601, 78602, 78603, 78604, 78605, 78606, 78607, 78608, 78609, 78610, 78611) c6( ) c7(bVP, bxw); #c1(KIF16B) c2(NP_078980) c3(kinesin-like protein KIF1 GB isoform 2) c4(216388, 216389) c5(216390, 216391, 216392, 216393, 216394, 216395) c6( ) c7(bVP, bxw); #c1(KIF1 GB) c2(XP_005260807) c3(kinesin-like protein KIF1 GB isoform X1) c4(276884) c5(276885, 276886, 276887, 276888, 276889, 276890, 276891, 276892, 276893, 276894, 276895, 276896, 276897, 276898, 276899, 276900) c6( ) c7(bVP, bxw); #c1(KIF1 GB) c2(XP_005260809) c3(kinesin-like protein KIF1 GB isoform X3) c4( ) c5(276901, 276902) c6( ) c7(bVP, bxw); #c1(KIF1 GB) c2(XP_005260811) c3(kinesin-like protein KIF1 GB isoform X6) c4(276903) c5(276904, 276905, 276906, 276907, 276908, 276909) c6( ) c7(bVP, bxw); #c1(KIF16B) c2(XP_006723651) c3(kinesin-like protein KIF16B isoform X7) c4(295268) c5(295269, 295270, 295271, 295272, 295273, 295274) c6( ) c7(bVP, bxw); #c1(KIF17) c2(XP_011540143) c3(kinesin-like protein KIF17 isoform X5) c4( ) c5(338501, 338502, 338503, 338504) c6( ) c7(vH, ND); #c1(KIF18A) c2(NP_112494) c3(kinesin-like protein KIF18A) c4(221158) c5(221159, 221160, 221161, 221162, 221163, 221164) c6( ) c7(T, aW, r, aT, ey, q); #c1(KIF1A) c2(NP_001230937) c3(kinesin-like protein KIF1A isoform 1) c4(87198) c5(87199, 87200, 87201, 87202, 87203, 87204) c6( ) c7(T, hl, aFa, bVQ, bVS, aj, bqQ, fu, bVR, cm, ik, akS, ad); #c1(KIF1A) c2(XP_005247080) c3(kinesin-like protein KIF1A isoform X3) c4(267637) c5(267638, 267639, 267640, 267641, 267642, 267643) c6( ) c7(T, hl, aFa, bVQ, bVS, aj, bqQ, fu, bVR, cm, ik, akS, ad); #c1(KIF1A) c2(XP_005247085) c3(kinesin-like protein KIF1A isoform X1) c4(267644) c5(267645, 267646, 267647, 267648, 267649, 267650, 267651) c6( ) c7(T, hl, aFa, bVQ, bVS, aj, bqQ, fu, bVR, cm, ik, akS, ad); #c1(KIF1A) c2(XP_011509666) c3(kinesin-like protein KIF1A isoform X2) c4(300245) c5(300246, 300247, 300248, 300249, 300250) c6( ) c7(T, hl, aFa, bVQ, bVS, aj, bqQ, fu, bVR, cm, ik, akS, ad); #c1(KIF1C) c2(NP_006603) c3(kinesin-like protein KIF1C) c4(166552, 166553) c5(166554, 166555, 166556, 166557, 166558, 166559, 166560, 166561, 166562, 166563, 166564, 166565, 166566) c6( ) c7(DS, Ng, no, ky, lu, bVT, Wl); #c1(KIF20A) c2(NP_005724) c3(kinesin-like protein KIF20A) c4(158861, 158862, 158863, 158864) c5(158865, 158866, 158867, 158868, 158869, 158870, 158871, 158872, 158873, 158874, 158875, 158876, 158877, 158878, 158879, 158880, 158881, 158882, 158883, 158884, 158885, 158886, 158887, 158888) c6( ) c7(T, aC, aE, Y, aA, n, kp, ey, aF); #c1(KIF20B) c2(NP_001271188) c3(kinesin-like protein KIF20B isoform 1) c4(103524, 103525, 103526) c5(103527, 103528, 103529, 103530, 103531, 103532, 103533, 103534, 103535, 103536, 103537, 103538, 103539, 103540, 103541, 103542, 103543, 103544) c6( ) c7(aW, aT, dL, Kw, bu, zy, el); #c1(KIF20B) c2(NP_057279) c3(kinesin-like protein KIF20B isoform 2) c4(189825, 189826, 189827) c5(189828, 189829, 189830, 189831, 189832, 189833, 189834, 189835, 189836, 189837, 189838, 189839, 189840, 189841, 189842, 189843, 189844, 189845) c6( ) c7(aW, aT, dL, Kw, bu, zy, el); #c1(KIF21A) c2(NP_001166934) c3(kinesin-like protein KIF21A isoform 3) c4(68657) c5(68658, 68659, 68660, 68661, 68662, 68663) c6( ) c7(Na, aUl, bVV, ft, tT, arH, oj, bVP, aNy, xv, bxw, bVU, zW, Al); #c1(KIF21A) c2(NP_00166935) c3(kinesin-like protein KIF21A isoform 1) c4(68664) c5(68665, 68666, 68667, 68668, 68669, 68670) c6( ) c7(Na, aUl, bVV, ft, tT, arH, oj, bVP, aNy, xv, bxw, bVU, zW, Al); #c1(KIF21A) c2(NP_001166936) c3(kinesin-like protein KIF21A isoform 4) c4(68671) c5(68672, 68673, 68674, 68675, 68676, 68677, 68678) c6( ) c7(Na, aUl, bVV, ft, tT, arH, oj, bVP, aNy, xv, bxw, bVU, zW, Al); #c1(KIF21A) c2(NP_060111) c3(kinesin-like protein KIF21A isoform 2) c4(194234) c5(194235, 194236, 194237, 194238, 194239, 194240) c6( ) c7(Na, aUl, bVV, ft, tT, arH, oj, bVP, aNy, xv, bxw, bVU, zW, Al); #c1(KIF21A) c2(XP_005269064) c3(kinesin-like protein KIF21A isoform X1) c4(282270) c5(282271, 282272, 282273, 282274, 282275, 282276) c6( ) c7(Na, aUl, bVV, ft, tT, arH, oj, bVP, aNy, xv, bxw, bVU, zW, Al); #c1(KIF21A) c2(XP_005269070) c3(kinesin-like protein KIF21A isoform X10) c4(282277) c5(282278, 282279, 282280, 282281, 282282, 282283) c6( ) c7(Na, aUl, bVV, ft, tT, arH, oj, bVP, aNy, xv, bxw, bVU, zW, Al); #c1(KIF21A) c2(XP_005269071) c3(kinesin-like protein KIF21A isoform X11) c4(282284) c5(282285, 282286, 282287, 282288, 282289, 282290, 282291) c6( ) c7(Na, aUl, bVV, ft, tT, arH, oj, bVP, aNy, xv, bxw, bVU, zW, Al); #c1(KIF21B) c2(NP_001239029) c3(kinesin-like protein KIF21B isoform 1) c4(88191, 88192) c5(88193, 88194, 88195, 88196, 88197, 88198, 88199, 88200, 88201, 88202, 88203, 88204) c6( ) c7(fe, V, mR, W, iV); #c1(KIF21B) c2(NP_001239031) c3(kinesin-like protein KIF21B isoform 3) c4(88205) c5(88206, 88207, 88208, 88209, 88210, 88211) c6( ) c7(fe, V, mR, W, iV); #c1(KIF21B) c2(NP_001239032) c3(kinesin-like protein KIF21B isoform 4) c4(88212) c5(88213, 88214, 88215, 88216, 88217, 88218) c6( ) c7(fe, V, mR, W, iV); #c1(KIF21B) c2(NP_060066) c3(kinesin-like protein KIF21B isoform 2) c4(194020) c5(194021, 194022, 194023, 194024, 194025, 194026) c6( ) c7(fe, V, mR, W, iV); #c1(KIF22) c2(NP_001243199) c3(kinesin-like protein KIF22 isoform 2) c4(90122) c5(90123, 90124, 90125, 90126, 90127, 90128, 90129, 90130, 90131, 90132, 90133, 90134, 90135) c6( ) c7(T, aA, bB, fr, adS, q, ad, av, aT, rE, ae, B, aW, oV, PX, r, lo, aF, t, ik, bz, bVW, bVX, eM, bE, m, X, qc); #c1(KIF22) c2(NP_015556) c3(kinesin-like protein KIF22 isoform 1) c4(171847, 171848) c5(171849, 171850, 171851, 171852, 171853, 171854, 171855, 171856, 171857, 171858, 171859) c6( ) c7(T, aA, bB, fr, adS, q, ad, av, aT, rE, ae, B, aW, oV, PX, r, lo, aF, t, ik, bz, bVW, bVX, eM, bE, m, X, qc); #c1(KIF23) c2(NP_004847) c3(kinesin-like protein KIF23 isoform 2) c4(150285) c5(150286, 150287, 150288, 150289, 150290, 150291) c6( ) c7(aW, Y, ey, eA, aT); #c1(KIF23) c2(NP_612565) c3(kinesin-like protein KIF23 isoform 1) c4(236691) c5(236692, 236693, 236694, 236695, 236696, 236697, 236698, 236699, 236700, 236701, 236702, 236703) c6( ) c7(aW, Y, ey, eA, aT); #c1(KIF23) c2(XP_005254856) c3(kinesin-like protein KIF23 isoform X4) c4(273343) c5(273344, 273345, 273346, 273347, 273348, 273349, 273350, 273351, 273352, 273353) c6( ) c7(aW, Y, ey, eA, aT); #c1(KIF23) c2(XP_011520541) c3(kinesin-like protein KIF23 isoform X6) c4(315468) c5( ) c6( ) c7(aW, Y, ey, eA, aT); #c1(KIF24) c2(NP_919289) c3(kinesin-like protein KIF24) c4( ) c5(258460, 258461, 258462, 258463, 258464, 258465, 258466, 258467, 258468, 258469, 258470, 258471, 258472, 258473, 258474, 258475, 258476, 258477, 258478) c6( ) c7(WY); #c1(KIF24) c2(XP_005251508) c3(kinesin-like protein KIF24 isoform X4) c4(270974) c5( ) c6( ) c7(WY); #c1(KIF24) c2(XP_011516164) c3(kinesin-like protein KIF24 isoform X2) c4(309408, 309409) c5(309410, 309411, 309412) c6( ) c7(WY); #c1(KIF25) c2(NP_005346) c3(kinesin-like protein KIF25 isoform 2) c4(155282) c5(155283, 155284, 155285, 155286, 155287, 155288) c6( ) c7(Ea); #c1(KIF25) c2(NP_085118) c3(kinesin-like protein KIF25 isoform 1) c4(219299) c5(219300, 219301, 219302, 219303, 219304, 219305, 219306, 219307, 219308, 219309) c6( ) c7(Ea); #c1(KIF26A) c2(NP_056471) c3(kinesin-like protein KIF26A) c4(187655) c5(187656, 187657, 187658, 187659, 187660, 187661) c6( ) c7(aC, ac); #c1(KIF26B) c2(NP_060482) c3(kinesin-like protein KIF26B) c4(196188) c5(196189, 196190, 196191, 196192, 196193, 196194) c6( ) c7(aW, j, ac, aT); #c1(KIF26B) c2(XP_011542520) c3(kinesin-like protein KIF26B isoform X3) c4(341060, 341061) c5(341062, 341063, 341064, 341065, 341066, 341067, 341068, 341069, 341070, 341071, 341072, 341073) c6( ) c7(aW, j, ac, aT); #c1(KIF2A) c2(NP_001091981) c3(kinesin-like protein KIF2A isoform 2) c4(39128) c5(39129, 39130, 39131, 39132, 39133, 39134) c6( ) c7(HQ, PK, dH, aW, aT, zN, aj, ey, QK, ae, bVY, J, ad); #c1(KIF2A) c2(NP_001230881) c3(kinesin-like protein KIF2A isoform 3) c4(86908) c5(86909, 86910, 86911, 86912, 86913, 86914, 86915, 86916, 86917, 86918, 86919, 86920) c6( ) c7(HQ, PK, dH, aW, aT, zN, aj, ey, QK, ae, bVY, J, ad); #c1(KIF2A) c2(NP_001230882) c3(kinesin-like protein KIF2A isoform 4) c4(86921, 86922) c5(86923, 86924, 86925, 86926, 86927, 86928) c6( ) c7(HQ, PK, dH, aW, aT, zN, aj, ey, QK, ae, bVY, J, ad); #c1(KIF2A) c2(NP_004511) c3(kinesin-like protein KIF2A isoform 1) c4(147047) c5(147048, 147049, 147050, 147051, 147052, 147053) c6( ) c7(HQ, PK, dH, aW, aT, zN, aj, ey, QK, ae, bVY, J, ad); #c1(KIF2B) c2(NP_115948) c3(kinesin-like protein KIF2B) c4(224881, 224882) c5(224883, 224884, 224885, 224886, 224887, 224888, 224889, 224890, 224891, 224892, 224893, 224894) c6( ) c7(tT, v); #c1(KIF2C) c2(NP_001284584) c3(kinesin-like protein KIF2C isoform 2) c4(112078, 112079, 112080) c5(112081, 112082, 112083, 112084, 112085, 112086, 112087, 112088, 112089, 112090, 112091, 112092) c6( ) c7(T, bE, aW, aw, aT, r, aA, aj, bB, j, ci, aF, bu, q, J); #c1(KIF2C) c2(NP_001284585) c3(kinesin-like protein KIF2C isoform 3) c4(112093) c5(112094, 112095, 112096, 112097, 112098, 112099) c6( ) c7(T, bE, aW, aw, aT, r, aA, aj, bB, j, ci, aF, bu, q, J); #c1(KIF2C) c2(XP_011538843) c3(kinesin-like protein KIF2C isoform X1) c4( ) c5(336903, 336904, 336905, 336906, 336907, 336908, 336909, 336910, 336911, 336912, 336913) c6( ) c7(T, bE, aW, aw, aT, r, aA, aj, bB, j, ci, aF, bu, q, J); #c1(KIF3A) c2(NP_001287720) c3(kinesin-like protein KIF3A isoform 1) c4(112656) c5(112657, 112658, 112659, 112660, 112661, 112662) c6( ) c7(bM, T, sS, tk, arS, Az, wB, h, zG, K, bVZ, pM, eE, aY, ae, ad); #c1(KIF3A) c2(NP_001287721) c3(kinesin-like protein KIF3A isoform 2) c4(112663) c5(112664, 112665, 112666, 112667, 112668, 112669) c6( ) c7(bM, T, sS, tk, arS, Az, wB, h, zG, K, bVZ, pM, eE, aY, ae, ad); #c1(KIF3A) c2(NP_008985) c3(kinesin-like protein KIF3A isoform 3) c4(170057, 170058) c5(170059, 170060, 170061, 170062, 170063, 170064, 170065, 170066, 170067, 170068, 170069, 170070) c6( ) c7(bM, T, sS, tk, arS, Az, wB, h, zG, K, bVZ, pM, eE, aY, ae, ad); #c1(KIF3B) c2(NP_004789) c3(kinesin-like protein KIF3B) c4(149871, 149872) c5(149873, 149874, 149875, 149876, 149877, 149878, 149879, 149880, 149881, 149882, 149883, 149884) c6( ) c7(B); #c1(KIF3C) c2(NP_002245) c3(kinesin-like protein KIF3C) c4(126875, 126876) c5(126877, 126878, 126879, 126880, 126881, 126882, 126883, 126884, 126885, 126886, 126887, 126888, 126889, 126890, 126891, 126892) c6( ) c7(cm, PJ); #c1(KIF4A) c2(XP_011529195) c3(chromosome-associated kinesin KIF4A isoform X1) c4(324602, 324603, 324604) c5(324605, 324606, 324607, 324608, 324609, 324610, 324611, 324612, 324613, 324614, 324615, 324616, 324617, 324618, 324619, 324620, 324621, 324622) c6( ) c7(T, eM, qt, ha, ey, nA, bz); #c1(KIF4B) c2(NP_001092763) c3(chromosome-associated kinesin KIF4B) c4(39629) c5(39630, 39631, 39632, 39633, 39634, 39635) c6( ) c7(fq, ac, cz); #c1(KIF5A) c2(NP_004975) c3(kinesin heavy chain isoform 5A) c4(151542, 151543) c5(151544, 151545, 151546, 151547, 151548, 151549, 151550, 151551, 151552, 151553, 151554, 151555) c6( ) c7(aiZ, bdn, bWa, A, aV, aW, bT, tT, ajb, aT, cL, bk, fu, bU, y, V, o, wD); #c1(KIF5B) c2(NP_004512) c3(kinesin-1 heavy chain) c4(147054) c5(147055, 147056, 147057, 147058, 147059, 147060) c6( ) c7(iu, eG, bWa, d, eW, aV, eM, Qu, iE, Y, pX, ff, r, dz, aj, eA, en, g, cm, bz, q); #c1(KIF5C) c2(NP_004513) c3(kinesin heavy chain isoform 5C) c4(147061, 147062, 147063, 147064) c5(147065, 147066, 147067, 147068, 147069, 147070, 147071, 147072, 147073, 147074, 147075, 147076, 147077, 147078, 147079, 147080, 147081, 147082, 147083, 147084, 147085, 147086, 147087, 147088, 147089, 147090, 147091) c6( ) c7(PK, bWb, FD, zN, nA, QK); #c1(KIF5C) c2(XP_011509459) c3(kinesin heavy chain isoform 5C isoform X1) c4(300039) c5(300040, 300041, 300042, 300043, 300044, 300045) c6( ) c7(PK, bWb, FD, zN, nA, QK); #c1(KIF6) c2(NP_001275949) c3(kinesin-like protein KIF6 isoform 2) c4(107500) c5(107501, 107502, 107503, 107504, 107505, 107506) c6( ) c7(bL, dD, ac, sO, ea, e, aw, j, aS, eq, cM, eu); #c1(KIF6) c2(NP_001275950) c3(kinesin-like protein KIF6 isoform 3) c4(107507) c5(107508, 107509, 107510, 107511, 107512, 107513) c6( ) c7(bL, dD, ac, sO, ea, e, aw, j, aS, eq, cM, eu); #c1(KIF6) c2(NP_001275953) c3(kinesin-like protein KIF6 isoform 4) c4( ) c5(107514, 107515, 107516, 107517, 107518, 107519, 107520, 107521, 107522, 107523, 107524, 107525, 107526, 107527, 107528, 107529, 107530, 107531) c6( ) c7(bL, dD, ac, sO, ea, e, aw, j, aS, eq, cM, eu); #c1(KIF6) c2(NP_659464) c3(kinesin-like protein KIF6 isoform 1) c4(240076, 240077, 240078) c5(240079, 240080, 240081, 240082, 240083, 240084, 240085, 240086, 240087, 240088, 240089, 240090) c6( ) c7(bL, dD, ac, sO, ea, e, aw, j, aS, eq, cM, eu); #c1(KIF6) c2(XP_011512659) c3(kinesin-like protein KIF6 isoform X2) c4(303649) c5( ) c6( ) c7(bL, dD, ac, sO, ea, e, aw, j, aS, eq, cM, eu); #c1(KIF6) c2(XP_011512661) c3(kinesin-like protein KIF6 isoform X4) c4(303650, 303651, 303652) c5(303653, 303654, 303655, 303656, 303657, 303658, 303659, 303660, 303661, 303662, 303663, 303664, 303665) c6( ) c7(bL, dD, ac, sO, ea, e, aw, j, aS, eq, cM, eu); #c1(KIF6) c2(XP_011512662) c3(kinesin-like protein KIF6 isoform X5) c4(303666, 303667, 303668) c5(303669, 303670, 303671, 303672, 303673, 303674) c6( ) c7(bL, dD, ac, sO, ea, e, aw, j, aS, eq, cM, eu); #c1(KIF6) c2(XP_011512663) c3(kinesin-like protein KIF6 isoform X6) c4( ) c5(303675, 303676, 303677, 303678, 303679, 303680, 303681, 303682, 303683, 303684, 303685, 303686) c6( ) c7(bL, dD, ac, sO, ea, e, aw, j, aS, eq, cM, eu); #c1(KIF7) c2(XP_005254959) c3(kinesin-like protein KIF7 isoform X1) c4(273417) c5(273418, 273419, 273420, 273421, 273422) c6( ) c7(Na, ben, Y, ry, bWc, cz, nA, Tp, Ll, bWd, bwD); #c1(KIF7) c2(XP_011519833) c3(kinesin-like protein KIF7 isoform X2) c4( ) c5(314575, 314576) c6( ) c7(Na, ben, Y, ry, bWc, cz, nA, Tp, Ll, bWd, bwD); #c1(KIF9) c2(NP_001128350) c3(kinesin-like protein KIF9 isoform 2) c4(49951) c5(49952, 49953, 49954, 49955, 49956, 49957, 49958, 49959, 49960, 49961, 49962) c6( ) c7(j); #c1(KIF9) c2(NP_071737) c3(kinesin-like protein KIF9 isoform 1) c4(211865, 211866) c5(211867, 211868, 211869, 211870, 211871, 211872, 211873, 211874, 211875, 211876, 211877, 211878, 211879, 211880, 211881, 211882) c6( ) c7(j); #c1(KIF9) c2(XP_005265445) c3(kinesin-like protein KIF9 isoform X2) c4( ) c5(279552, 279553, 279554) c6( ) c7(j); #c1(KIF9) c2(XP_005265446) c3(kinesin-like protein KIF9 isoform X4) c4( ) c5(279555, 279556) c6( ) c7(j); #c1(KIF9) c2(XP_011532305) c3(kinesin-like protein KIF9 isoform X3) c4(328473) c5( ) c6( ) c7(j); #c1(KIFAP3) c2(NP_001191443) c3(kinesin-associated protein 3 isoform 2) c4(81538, 81539) c5(81540, 81541, 81542, 81543, 81544, 81545) c6( ) c7(bA, Vd, ad, c); #c1(KIFAP3) c2(NP_001191445) c3(kinesin-associated protein 3 isoform 3) c4(81546) c5(81547, 81548, 81549, 81550, 81551, 81552) c6( ) c7(bA, Vd, ad, c); #c1(KIFAP3) c2(NP_001191446) c3(kinesin-associated protein 3 isoform 4) c4( ) c5(81553, 81554, 81555, 81556, 81557, 81558, 81559, 81560, 81561, 81562, 81563) c6( ) c7(bA, Vd, ad, c); #c1(KIFAP3) c2(NP_055785) c3(kinesin-associated protein 3 isoform 1) c4(183477, 183478) c5(183479, 183480, 183481, 183482, 183483, 183484, 183485) c6( ) c7(bA, Vd, ad, c); #c1(KIFC1) c2(NP_002254) c3(kinesin-like protein KIFC1) c4(126969) c5(126970, 126971, 126972, 126973, 126974, 126975, 126976, 126977, 126978, 126979, 126980, 126981, 126982) c6( ) c7(eZ, qy); #c1(KIFC1) c2(XP_011512889) c3(kinesin-like protein KIFC1 isoform X6) c4(304261, 304262) c5(304263, 304264, 304265, 304266, 304267, 304268, 304269, 304270, 304271, 304272, 304273, 304274, 304275, 304276, 304277) c6( ) c7(eZ, qy); #c1(KIFC3) c2(NP_001123572) c3(kinesin-like protein KIFC3 isoform 2) c4( ) c5(47614, 47615, 47616, 47617, 47618, 47619, 47620, 47621, 47622, 47623, 47624, 47625, 47626, 47627, 47628) c6( ) c7(nw, Tp); #c1(KIFC3) c2(NP_005541) c3(kinesin-like protein KIFC3 isoform 1) c4(157248) c5(157249, 157250, 157251, 157252, 157253, 157254) c6( ) c7(nw, Tp); #c1(KIFC3) c2(XP_005255994) c3(kinesin-like protein KIFC3 isoform X5) c4( ) c5(273910) c6( ) c7(nw, Tp); #c1(KIN) c2(NP_036443) c3(DNA/RNA-binding protein KIN17) c4(173921) c5(173922, 173923, 173924, 173925, 173926, 173927) c6( ) c7(aW, aT); #c1(KIN) c2(XP_006717497) c3(DNA/RNA-binding protein KIN17 isoform X1) c4( ) c5(291154) c6( ) c7(aW, al); #c1(KIN) c2(XP_006717498) c3(DNA/RNA-binding protein KIN17 isoform X3) c4(291155) c5(291156, 291157, 291158, 291159, 291160) c6( ) c7(aW, aT); #c1(KIN) c2(XP_011517729) c3(DNA/RNA-binding protein KIN17 isoform X2) c4(311521) c5(311522, 311523, 311524, 311525, 311526, 311527) c6( ) c7(aW, aT); #c1(KIR2DL1) c2(NP_055033) c3(killer cell immunoglobulin-like receptor 2DLI precursor) c4(178410) c5(178411, 178412, 178413, 178414, 178415, 178416, 178417, 178418) c6( ) c7(bL, fR, aD, iW, aw, al, jx, fN, jE, qN, V, wD, dP, x, AW, ga, F, ds, hD, rN, h, rB, hH, A, B, mR, iU, aAT, HA, ih, cr, eZ, aq, vs, hp, ve, uC, cM, bPK, cm, iP, ck, eU, sn, ac, iT, blS, bbD, k, aCD, y, bt, ne, hv, avY); #c1(KIR2DL2) c2(NP_055034) c3(killer cell immunoglobulin-like receptor 2DL2 precursor) c4(178419, 178420) c5(178421, 178422, 178423, 178424, 178425, 178426, 178427, 178428, 178429, 178430, 178431, 178432) c6( ) c7(aD, iW, aw, al, jx, fN, aj, lR, jE, qN, fr, V, eW, J, wD, bBq, AW, ga, F, hD, rN, h, JK, rB, hH, A, B, mR, iU, Y, aAT, HA, ih, cr, eZ, aq, lo, vs, hp, iV, cm, cM, bPK, uC, pb, iP, ck, eU, sn, ac, iT, bJa, aED, eo, k, aCD, gg, y, bt, ne, hv, vi); #c1(KIR2DL3) c2(NP_056952) c3(killer cell immunoglobulin-like receptor 2DL3 precursor) c4(188182) c5(188183, 188184, 188185, 188186, 188187, 188188, 188189) c6( ) c7(bL, Kd, T, aD, iW, aw, al, jx, fN, fd, jh, aj, lR, jE, qN, aFV, fr, V, eh, eA, amW, J, hX, dP, bBq, AW, ga, F, wD, dV, hD, rN, h, JK, rB, DY, hH, A, B, mR, iU, Y, aAT, HA, ih, cr, eZ, aq, vs, hp, iV, ve, cM, bPK, uC, pb, cm, ck, eU, sn, ac, zr, iT, bJa, aED, bt, k, aCD, y, lB, ne, hv, vi); #c1(KIR2DL4) c2(NP_001074239) c3(killer cell immunoglobulin-like receptor 2DL4 isoform c precursor) c4(37015, 37016, 37017, 37018) c5(37019, 37020, 37021, 37022, 37023, 37024, 37025, 37026, 37027, 37028, 37029, 37030, 37031, 37032, 37033, 37034, 37035, 37036, 37037, 37038, 37039, 37040, 37041, 37042, 37043) c6( ) c7(T, aD, al, jx, jh, eb, jE, qN, V, mC, wD, hX, AW, ga, F, rN, h, rB, hH, A, B, mR, s, aAT, HA, ih, cr, eZ, aq, hp, uC, cM, iP, ck, sn, iT, fL, aCD, y, bt, ne, VB); #c1(KIR2DL4) c2(NP_001074241) c3(killer cell immunoglobulin-like receptor 2DL4 isoform b precursor) c4( ) c5(37044)37045, 37046, 37047, 37048) c6( ) c7(T, aD, al, jx, jh, eb, jE, qN, V, mC, wD, hX, AW, ga, F, rN, h, rB, hH, A, B, mR, s, aAT, HA, ih, cr, eZ, aq, hp, uC, cM, iP, ck, sn, iT, fL, aCD, y, bt, ne, VB); #c1(KIR2DL4) c2(NP_002246) c3(killer cell immunoglobulin-like receptor 2DL4 isoform a precursor) c4(126893, 126894, 126895, 126896, 126897, 126898, 126899) c5(126900, 126901, 126902, 126903, 126904, 126905, 126906, 126907, 126908, 126909, 126910, 126911, 126912, 126913, 126914, 126915, 126916, 126917, 126918, 126919, 126920, 126921, 126922, 126923, 126924, 126925, 126926, 126927, 126928, 126929, 126930, 126931, 126932) c6( ) c7(T, aD, al, jx, jh, eb, jE, qN, V, mC, wD, hX, AW, ga, F, rN, h, rB, hH, A, B, mR, s, aAT, HA, ih, cr, eZ, aq, hp, uC, cM, iP, ck, sn, iT, fL, aCD, y, bt, ne, VB); #c1(KIR2DL5A) c2(NP_065396) c3(killer cell immunoglobulin-like receptor 2DL5A precursor) c4(204727, 204728) c5(204729, 204730, 204731, 204732, 204733, 204734, 204735, 204736, 204737, 204738, 204739, 204740, 204741) c6( ) c7(bL, T, aD, iW, axd, al, jx, jh, v, jE, oL, V, amW, wD, aMp, dP, hX, AW, ga, F, x, gd, dV, hD, rN, h, rB, hH, A, B, mR, iU, aAT, HA, ih, cr, eZ, aq, lo, hp, pL, cM, pb, aYt, Cv, ck, sn, W, Jr, aWZ, ig, iT, dW, k, aCD, y, bt, ne); #c1(KIR2DL5B) c2(NP_001018091) c3(killer cell immunoglobulin-like receptor 2DL5B precursor) c4(27310) c5(27311) c6( ) c7(ck, A, mR, Jr, aAT, eZ, rN, h, lo, hp, bt); #c1(KIR2DS1) c2(NP_055327) c3(killer cell immunoglobulin-like receptor 20SI precursor) c4(180520) c5( ) c6( ) c7(bL, T, aD, iW, axd, al, aw, fN, jh, v, jE, qN, oL, V, amW, wD, aMp, dP, hX, AW, ga, F, x, gd, dV, hD, rN, h, rB, hH, A, B, mR, iU, aAT, aFE, HA, ih, cr, eZ, aq, lo, vs, hp, pL, cm, cM, uC, pb, aYt, Cv, ck, sn, W, Jr, aWZ, ac, ig, iT, dW, k, aCD, y, bt, ne, hv, avY); #c1(KIR2DS2) c2(NP_001278624) c3(killer cell immunoglobulin-like receptor 2DS2 isoform b precursor) c4( ) c5(109903, 109904, 109905, 109906) c6( ) c7(Kd, T, aD, iW, aw, al, jx, jh, avY, fd, aj, lR, jE, fr, aFV, V, eh, eA, amW, J, hX, dP, bBq, AW, wD, F, dV, hD, rN, h, JK, rB, DY, hH, A, B, mR, iU, aAT, HA, ih, cr, eZ, aq, vs, cm, cM, pb, Cv, ck, sn, ac, zr, iT, bJa, k, aCD, y, bt, ne, hv, vi); #c1(KIR2DS2) c2(NP_001278625) c3(killer cell immunoglobulin-like receptor 2DS2 isoform c precursor) c4( ) c5(109907) c6( ) c7(Kd, T, aD, iW, aw, al, jx, jh, avY, fd, aj, lR, jE, fr, aFV, V, eh, eA, amW, J, hX, dP, bBq, AW, wD, F, dV, hD, rN, h, JK, rB, DY, hH, A, B, mR, iU, aAT, HA, ih, cr, eZ, aq, vs, cm, cM, pb, Cv, ck, sn, ac, zr, iT, bJa, k, aCD, y, bt, ne, hv, vi); #c1(KIR2DS2) c2(XP_006725842) c3(killer cell immunoglobulin-like receptor 2DS2 isoform X8) c4(297005) c5(297006, 297007, 297008, 297009, 297010, 297011, 297012, 297013) c6( ) c7(Kd, T, aD, iW, aw, al, jx, jh, avY, fd, aj, lR, jE, fr, aFV, V, eh, eA, amW, J, hX, dP, bBq, AW, wD, F, dV, hD, rN, h, JK, rB, DY, hH, A, B, mR, iU, aAT, HA, ih, cr, eZ, aq, vs, cm, cM, pb, Cv, ck, sn, ac, zr, iT, bJa, k, aCD, y, bt, ne, hv, vi); #c1(KIR2DS3) c2(NP_036445) c3(killer cell immunoglobulin-like receptor 2DS3 precursor) c4(173928) c5(173929) c6( ) c7(Kd, aD, iW, al, jx, fN, jE, qN, V, amW, wD, hX, AW, ga, F, hD, rN, h, rB, hH, A, B, mR, iU, aAT, aFE, HA, ih, cr, eZ, aq, hp, cm, uC, cM, aYt, Cv, ck, sn, iT, blS, dc, k, aCD, y, bt, ne, avY); #c1(KIR2DS4) c2(NP_036446) c3(killer cell immunoglobulin-like receptor 2DS4 isoform 1 precursor) c4(173930) c5( ) c6( ) c7(bL, aD, iW, axd, al, jx, fN, jh, jE, qN, oL, aY, V, wD, hX, AW, ga, F, ajk, bFU, dV, hD, rN, h, rB, hH, A, B, mR, iU, Y, aAT, HA, ih, cr, eZ, aq, hp, pL, cm, uC, cM, ck, sn, iT, Jr, aED, k, aCD, y, bt, ne, avY); #c1(KIR3DL1) c2(NP_037421) c3(killer cell immunoglobulin-like receptor 3DL1 precursor) c4(175412, 175413, 175414, 175415, 175416, 175417, 175418) c5(175419, 175420, 175421, 175422, 175423, 175424, 175425, 175426, 175427, 175428, 175429, 175430, 175431, 175432, 175433, 175434, 175435, 175436, 175437, 175438, 175439, 175440, 175441, 175442, 175443, 175444, 175445, 175446, 175447, 175448, 175449, 175450, 175451, 175452, 175453, 175454, 175455, 175456, 175457, 175458, 175459, 175460, 175461, 175462, 175463, 175464, 175465, 175466, 175467, 175468, 175469) c6(175470, 175471, 175472, 175473) c7(T, fR, al, fd, aeu, lR, amW, x, AW, dV, rB, hH, aW, aAT, HA, ih, hN, cm, ve, uC, bw, iP, iT, Jr, aWZ, dW, k, pw, avY, fN, n, aj, fr, qN, qW, aMp, bBq, gd, h, A, mR, aq, cM, W, eo, fL, aCD, B, bL, jv, jx, v, V, J, ga, akU, ld, aT, iU, Y, WO, lo, pb, bN, Cv, ck, sn, ig, oX, yH, bt, vi, aD, iW, axd, jh, jE, mC, wD, hX, dP, F, hD, rN, lQ, aFE, cr, eZ, hp, bPK, dr, aYt, gk, zr, blS, y, fh); #c1(KIR3DL2) c2(NP_001229796) c3(killer cell immunoglobulin-like receptor 3DL2 isoform 2 precursor) c4(85256) c5(85257) c6( ) c7(aD, al, jE, qN, V, ai, wD, AW, ga, F, il, rN, h, rB, hH, A, B, mR, aAT, HA, ih, cr, eZ, aq, hp, ve, uC, cM, ck, eU, sn, iT, aCD, y, bt, ne); #c1(KIR3DL2) c2(NP_006728) c3(killer cell immunoglobulin-like receptor 3DL2 isoform 1 precursor) c4(167484) c5(167485, 167486, 167487, 167488, 167489, 167490, 167491) c6( ) c7(aD, al, jE, qN, V, ai, wD, AW, ga, F, il, rN, h, rB, hH, A, B, mR, aAT, HA, ih, cr, eZ, aq, hp, ve, uC, cM, ck, eU, sn, iT, aCD, y, bt, ne); #c1(KIR3DL2) c2(XP_011545872) c3(killer cell immunoglobulin-like receptor 3DL2 isoform X2) c4(344775) c5( ) c6( ) c7(aD, al, jE, qN, V, ai, wD, AW, ga, F, il, rN, h, rB, hH, A, B, mR, aAT, HA, ih, cr, eZ, aq, hp, ve, uC, cM, ck, eU, sn, iT, aCD, y, bt, ne); #c1(KIR3DL2) c2(XP_011545882) c3(killer cell immunoglobulin-like receptor 3DL2 isoform X1) c4(344776) c5(344777, 344778, 344779, 344780, 344781, 344782, 344783) c6( ) c7(aD, al, jE, qN, V, ai, wD, AW, ga, F, il, rN, h, rB, hH, A, B, mR, aAT, HA, ih, cr, ez, aq, hp, ve, uC, cM, ck, eU, sn, iT, aCD, y, bt, ne); #c1(KIR3DL2) c2(XP_011545883) c3(killer cell immunoglobulin-like receptor 3DL2 isoform X2) c4( ) c5(344784, 344785, 344786, 344787, 344788, 344789) c6( ) c7(aD, al, jE, qN, V, ai, wD, AW, ga, F, il, rN, h, rB, hH, A, B, mR, aAT, HA, ih, cr, eZ, aq, hp, ve, uC, cM, ck, eU, sn, iT, aCD, y, bt, ne); #c1(KIR3DL3) c2(NP_703144) c3(killer cell immunoglobulin-like receptor 3DL3 precursor) c4(245976, 245977) c5(245978, 245979, 245980, 245981, 245982, 245983, 245984, 245985, 245986, 245987, 245988, 245989, 245990) c6( ) c7(Kd, T, aD, al, jx, jh, fd, aj, lR, jE, qN, aFV, fr, V, eh, eA, amW, J, hX, dP, bBq, AW, ga, F, wD, dV, rN, h, JK, rB, DY, hH, A, B, mR, iU, aAT, HA, ih, cr, eZ, aq, hp, uC, cM, pb, ck, sn, zr, iT, bJa, aCD, y, bt, ne, hv, vi); #c1(KIRREL) c2(NP_001273278) c3(kin of IRRE-like protein 1 isoform 2 precursor) c4(104580, 104581) c5(104582, 104583, 104584, 104585, 104586, 104587, 104588, 104589, 104590, 104591, 104592, 104593, 104594, 104595, 104596, 104597, 104598, 104599) c6( ) c7(sk, A, ah, eR); #c1(KIRREL) c2(NP_060710) c3(kin of IRRE-like protein 1 isoform 1 precursor) c4(197327, 197328) c5(197329, 197330, 197331, 197332, 197333, 197334) c6( ) c7(sk, A, ah, eR); #c1(KIRREL) c2(XP_005245362) c3(kin of IRRE-like protein 1 isoform X1) c4(266483) c5(266484, 266485, 266486, 266487, 266488, 266489) c6( ) c7(sk, A, ah, eR); #c1(KIRREL2) c2(NP_115499) c3(kin of IRRE-like protein 2 isoform a precursor) c4(222946) c5(222947, 222948, 222949, 222950, 222951, 222952) c6( ) c7(asp); #c1(KIRREL2) c2(NP_954648) c3(kin of IRRE-like protein 2 isoform b precursor) c4(261155) c5(261156, 261157, 261158, 261159, 261160, 261161) c6( ) c7(asp); #c1(KIRREL2) c2(NP_954649) c3(kin of IRRE-like protein 2 isoform c precursor) c4(261162, 261163) c5(261164, 261165, 261166, 261167, 261168, 261169, 261170, 261171, 261172, 261173, 261174, 261175) c6( ) c7(asp); #c1(KIRREL2) c2(XP_011525666) c3(kin of IRRE-like protein 2 isoform X2) c4(320081) c5(320082, 320083, 320084, 320085, 320086, 320087) c6( ) c7(asp); #c1(KIRREL3) c2(NP_001155179) c3(kin of IRRE-like protein 3 isoform 2 precursor) c4(61505) c5(61506, 61507, 61508, 61509, 61510, 61511, 61512, 61513) c6( ) c7(ac, alv, nA, Wl, he, cd, bWe); #c1(KIRREL3) c2(NP_115920) c3(kin of IRRE-like protein 3 isoform 1 precursor) c4(224694) c5(224695, 224696, 224697, 224698, 224699, 224700, 224701, 224702) c6( ) c7(ac, alv, nA, Wl, he, cd, bWe); #c1(KIRREL3) c2(XP_011541335) c3(kin of IRRE-like protein 3 isoform X2) c4(339925) c5(339926, 339927, 339928, 339929, 339930, 339931, 339932, 339933, 339934) c6( ) c7(ac, alv, nA, Wl, he, cd, bWe); #c1(KIRREL3) c2(XP_011541336) c3(kin of IRRE-like protein 3 isoform X7) c4(339935) c5(339936, 339937, 339938, 339939, 339940, 339941, 339942) c6( ) c7(ac, alv, nA, Wl, he, cd, bWe); #c1(KISS1) c2(XP_011507827) c3(metastasis-suppressor KiSS-1 isoform X1) c4(297971) c5(297972, 297973, 297974, 297975, 297976, 297977, 297978, 297979) c6( ) c7(T, dD, id, sw, ea, aA, cz, n, bB, v, j, ci, eq, fr, AO, q, ad, eJ, UE, yn, cc, aE, il, Hk, aT, dL, K, nJ, ae, eL, cp, yo, kl, CN, s, qt, aW, Y, ff, r, aF, en, cl, g, t, ik, bWf, eM, bE, AV, hL, hR, m, UF, B); #c1(KISS1R) c2(NP_115940) c3(kiSS-1 receptor) c4(224808) c5(224809, 224810, 224811, 224812, 224813, 224814) c6( ) c7(T, dD, id, dA, ea, bWg, aA, eq, ci, fr, yn, UE, aT, K, nJ, yo, CN, aW, Y, ff, bWh, yq, en, cl, aMP, eM, nB, hL, hR, UF, B); #c1(KIT) c2(NP_000213) c3(mast/stem cell growth factor receptor Kit isoform 1 precursor) c4(4635) c5(4636, 4637, 4638, 4639, 4640, 4641) c6( ) c7(Fv, bM, T, aC, aJV, fd, bB, qj, ci, asr, x, bv, bWj, bW, pX, aeO, bbu, cp, Qe, aa, auQ, yo, aeR, aAu, aW, wp, Ls, CT, Qm, g, as, eF, cm, bWs, QA, Qt, arZ, iP, bw, eM, nB, iA, iT, go, oy, vA, dW, k, m, bsS, aHk, Th, Le, ik, gU, Uc, aEj, wu, iB, WA, NT, qy, n, awr, fr, zP, JW, br, ft, pe, Tf, Xe, Nn, bkP, KN, ic, UD, apG, dU, aDC, Dz, Xh, aBl, dx, Nr, kj, Tc, AQ, Lh, ahJ, aq, po, bWt, pm, ban, XT, op, yR, Ld, Qf, bP, dB, hk, bWq, eD, bo, Vy, mg, hj, pg, bOQ, ph, aPG, aAE, fM, jx, aA, hz, afK, v, j, pd, q, er, eA, Zp, aRM, lg, hW, qK, bWn, aT, vH, dz, rO, bK, Tj, j, aPF, ae, ne, bA, jf, iF, hM, s, ND, vT, iU, Y, Lf, apK, r, en, aom, hf, t, KQ, bu, f, cl, aEp, zJ, ck, cT, Ta, nM, Dy, kP, hA, bWk, QG, hc, nG, gX, aws, bWl, zu, bt, On, kh, rQ, bWi, jN, bs, jh, bWp, hF, iJ, ad, hX, iC, av, bkq, fX, ib, py, Db, vJ, bWr, oW, Ti, hP, wm, fb, iL, bWo, aeQ, d, et, ff, eH, aeV, aF, aiz, aTa, adj, bz, aTb, dr, Hp, Nq, es, bF, baa, Ew, bE, asq, X, akC, apv, Lb, acH, bWm, jr, pC); #c1(KIT) c2(NP_001087241) c3(mast/stem cell growth factor receptor Kit isoform 2 precursor) c4(38303, 38304) c5(38305, 38306, 38307, 38308, 38309, 38310, 38311, 38312, 38313, 38314, 38315, 38316) c6( ) c7(Fv, bM, T, aC, aJV, fd, bB, qj, ci, asr, x, bv, bWj, bW, pX, aeO, bbu, cp, Qe, aa, auQ, yo, aeR, aAu, aW, wp, Ls, CT, Qm, g, as, eF, cm, bWs, QA, Qt, arZ, iP, bw, eM, nB, iA, iT, go, oy, vA, dW, k, m, bsS, aHk, Th, Le, ik, gU, Uc, aEj, wu, iB, WA, NT, qy, n, awr, fr, zP, JW, br, ft, pe, Tf, Xe, Nn, bkP, KN, ic, UD, apG, dU, aDC, Dz, Xh, aBl, dx, Nr, kj, Tc, AQ, Lh, ahJ, aq, po, bWt, pm, ban, XT, op, yR, Ld, Qf, bP, dB, hk, bWq, eD, bo, Vy, mg, hj, pg, bOQ, ph, aPG, aAE, fM, jx, aA, hz, afK, v, j, pd, q, er, eA, Zp, aRM, lg, hW, qK, bWn, aT, vH, dz, rO, bK, Tj, jj, aPF, ae, ne, bA, jf, iF, hM, s, ND, vT, iU, Y, Lf, apK, r, en, aom, hf, t, KQ, bu, f, cl, aEp, zJ, ck, cT, Ta, nM, Dy, kP, hA, bWk, QG, hc, nG, gX, aws, bWl, zu, bt, On, kh, rQ, bWi, jN, bs, jh, bWp, hF, iJ, ad, hX, iC, av, bkq, fX, ib, py, Db, vJ, bWr, oW, Ti, hP, wm, fb, iL, bWo, aeQ, d, et, ff, eH, aeV, aF, aiz, aTa, adj, bz, aTb, dr, Hp, Nq, es, bF, baa, Ew, bE, asq, X, aKC, apv, Lb, aeH, bWm, jr, pC); #c1(KITLG) c2(NP_000890) c3(kit ligand isoform b precursor) c4(16860, 16861) c5(16862, 16863, 16864, 16865, 16866, 16867, 16868, 16869, 16870, 16871, 16872, 16873, 16874, 16875, 16876, 16877, 16878, 16879, 16880, 16881, 16882, 16883, 16884, 16885, 16886, 16887, 16888, 16889, 16890, 16891, 16892, 16893, 16894, 16895, 16896, 16897, 16898, 16899, 16900, 16901, 16902, 16903, 16904, 16905, 16906, 16907, 16908, 16909, 16910, 16911, 16912, 16913, 16914, 16915, 16916, 16917, 16918, 16919, 16920, 16921, 16922, 16923, 16924, 16925, 16926, 16927, 16928, 16929, 16930, 16931, 16932, 16933, 16934, 16935, 16936, 16937, 16938, 16939, 16940, 16941, 16942, 16943, 16944, 16945, 16946, 16947, 16948, 16949, 16950, 16951, 16952, 16953, 16954) c6(16955) c7(Fv, bM, T, os, Js, fV, if, fd, qj, ci, Qp, x, bv, aE, il, K, cp, eG, zQ, aW, eE, eF, cm, bw, eM, nB, Kv, iA, U, k, m, gU, Uc, wu, fr, zP, ai, br, apG, dU, bl, HY, aq, zZ, Ld, ok, aPG, B, Jo, dA, oe, afK, j, q, aT, vH, rO, dz, aPF, kl, bWu, ND, vT, iU, Y, Lf, r, en, bu, cl, t, ck, Ta, c, hc, bt, On, bs, oZ, iJ, ad, dP, av, hd, CJ, ib, wm, fb, ff, xs, eH, avG, iR, bz, Ew, zr, Lb, y, pC); #c1(KITLG) c2(NP_003985) c3(kit ligand isoform a precursor) c4(142645) c5(142646, 142647, 142648, 142649, 142650, 142651, 142652, 142653, 142654) c6( ) c7(Fv, bM, T, os, Js, fV, if, fd, qj, ci, Qp, x, bv, aE, il, K, cp, eG, zQ, aW, eE, eF, cm, bw, eM, nB, Kv, iA, U, k, m, gU, Uc, wu, fr, zP, ai, br, apG, dU, bl, HY, aq, zZ, Ld, ok, aPG, B, Jo, dA, oe, afK, j, q, aT, vH, rO, dz, aPF, kl, bWu, ND, vT, iU, Y, Lf, r, en, bu, cl, t, ck, Ta, c, hc, bt, On, bs, oZ, iJ, ad, dP, av, hd, CJ, ib, wm, fb, ff, xs, eH, avG, iR, bz, Ew, zr, Lb, y, pC); #c1(KL) c2(NP_004786) c3(klotho precursor) c4(149629) c5(149630, 149631, 149632, 149633, 149634, 149635, 149636, 149637, 149638, 149639, 149640, 149641, 149642, 149643, 149644, 149645, 149646, 149647, 149648, 149649, 149650, 149651, 149652, 149653, 149654, 149655, 149656, 149657, 149658, 149659, 149660, 149661, 149662, 149663, 149664, 149665, 149666, 149667, 149668, 149669, 149670, 149671, 149672, 149673, 149674, 149675, 149676, 149677, 149678, 149679, 149680, 149681, 149682, 149683, 149684, 149685, 149686, 149687, 149688, 149689, 149690, 149691, 149692, 149693, 149694, 149695, 149696, 149697, 149698, 149699, 149700, 149701, 149702, 149703, 149704, 149705, 149706, 149707, 149708, 149709, 149710, 149711, 149712, 149713, 149714, 149715, 149716, 149717, 149718, 149719, 149720, 149721, 149722, 149723, 149724, 149725, 149726, 149727, 149728, 149729, 149730, 149731, 149732, 149733, 149734, 149735, 149736, 149737, 149738, 149739, 149740, 149741, 149742, 149743, 149744, 149745, 149746, 149747, 149748, 149749, 149750, 149751, 149752, 149753, 149754, 149755, 149756, 149757, 149758, 149759, 149760, 149761, 149762, 149763, 149764, 149765, 149766, 149767, 149768, 149769, 149770, 149771, 149772, 149773, 149774, 149775, 149776, 149777, 149778, 149779, 149780, 149781, 149782, 149783, 149784, 149785, 149786, 149787, 149788, 149789, 149790, 149791, 149792, 149793, 149794, 149795, 149796, 149797, 149798, 149799, 149800, 149801, 149802, 149803, 149804, 149805, 149806, 149807, 149808, 149809, 149810, 149811, 149812, 149813, 149814, 149815, 149816, 149817, 149818, 149819, 149820, 149821, 149822, 149823, 149824, 149825, 149826, 149827, 149828, 149829, 149830, 149831, 149832, 149833, 149834, 149835, 149836, 149837, 149838, 149839, 149840, 149841, 149842, 149843, 149844, 149845, 149846, 149847, 149848, 149849, 149850, 149851, 149852, 149853, 149854, 149855, 149856, 149857, 149858, 149859, 149860) c6(149861) c7(Fv, T, fR, ea, if, e, al, bB, aY, sD, aE, M, K, dQ, eN, azh, Hl, aW, bsZ, zC, rq, bTq, iS, uo, Hb, m, lS, uL, dD, sQ, aj, fr, qN, JW, ft, cc, zw, acm, jy, eV, bta, hi, Dg, aq, wU, afc, ut, B, bL, AB, eR, oe, eX, cz, cR, v, j, amC, V, q, J, eJ, MR, aT, cy, ae, bA, s, Y, r, rs, t, AA, asw, aOx, rR, ac, dh, fP, dy, qb, eq, ad, fU, Dj, ji, bsR, lQ, yh, vj, dq, Hl, bz, dr, qa, adA, OG, bE, bon, ei, y, bsV, aAN); #c1(KL) c2(XP_006719958) c3(klotho isoform X1) c4(293100) c5(293101, 293102, 293103, 293104, 293105, 293106, 293107) c6( ) c7(Fv, T, fR, ea, if, e, al, bB, aY, sD, aE, M, K, dQ, eN, azh, Hl, aW, bsZ, zC, rq, bTq, iS, uo, Hb, m, lS, uL, dD, sQ, aj, fr, qN, JW, ft, cc, zw, acm, jy, eV, bta, hi, Dg, aq, wU, afc, ut, B, bL, AB, eR, oe, eX, cz, cR, v, j, amC, V, q, J, eJ, MR, aT, cy, ae, bA, s, Y, r, rs, t, AA, asw, aOx, rR, ac, dh, fP, dy, qb, eq, ad, fU, Dj, ji, bsR, lQ, yh, vj, dq, Hl, bz, dr, qa, adA, OG, bE, bon, ei, y, bsV, aAN); #c1(KLB) c2(NP_783864) c3(beta-klotho) c4(251778) c5(251779, 251780, 251781, 251782, 251783, 251784, 251785, 251786, 251787, 251788, 251789, 251790, 251791, 251792, 251793, 251794, 251795, 251796, 251797, 251798, 251799, 251800, 251801, 251802, 251803, 251804, 251805, 251806, 251807, 251808, 251809, 251810, 251811, 251812, 251813, 251814, 251815, 251816, 251817, 251818, 251819, 251820, 251821, 251822, 251823, 251824, 251825, 251826, 251827, 251828, 251829, 251830, 251831, 251832, 251833, 251834, 251835, 251836, 251837, 251838, 251839, 251840, 251841, 251842, 251843, 251844, 251845, 251846, 251847, 251848, 251849, 251850, 251851, 251852, 251853, 251854, 251855, 251856, 251857, 251858, 251859, 251860, 251861, 251862, 251863, 251864, 251865, 251866, 251867, 251868, 251869, 251870) c6( ) c7(T, aZ, cz, h, KO, aq, v, bWv, ae, B, ad); #c1(KLC1) c2(NP_001123579) c3(kinesin light chain 1 isoform 3) c4(47737, 47738, 47739, 47740, 47741, 47742, 47743, 47744) c5(47745, 47746, 47747, 47748, 47749, 47750, 47751, 47752, 47753, 47754, 47755, 47756, 47757, 47758, 47759, 47760, 47761, 47762, 47763, 47764, 47765, 47766, 47767, 47768, 47769, 47770, 47771, 47772, 47773, 47774) c6( ) c7(ac, hk, Y, aWG, sW, cz, Da, V, af, Hv); #c1(KLC1) c2(NP_005543) c3(kinesin light chain 1 isoform 1) c4(157263, 157264, 157265, 157266) c5(157267, 157268, 157269, 157270, 157271, 157272, 157273, 157274, 157275, 157276, 157277, 157278, 157279, 157280, 157281, 157282, 157283, 157284, 157285, 157286, 157287, 157288, 157289, 157290, 157291, 157292, 157293, 157294, 157295, 157296, 157297, 157298, 157299, 157300) c6( ) c7(ac, hk, Y, aWG, sW, cz, Da, V, af, Hv); #c1(KLC1) c2(NP_891553) c3(kinesin light chain 1 isoform 2) c4(257298) c5(257299, 257300, 257301, 257302, 257303, 257304, 257305, 257306, 257307, 257308, 257309, 257310, 257311, 257312, 257313, 257314, 257315, 257316) c6( ) c7(ac, hk, Y, aWG, sW, cz, Da, V, af, Hv); #c1(KLF1) c2(NP_006554) c3(Krueppel-like factor 1) c4(166194) c5(166195, 166196, 166197, 166198, 166199, 166200) c6( ) c7(bL, ck, BU, VJ, avS, qb, Fm, bWw, adw, oW, oY, aot, bfX, S, pw, avG, bt, bWx); #c1(KLF10) c2(NP_001027453) c3(Krueppel-like factor 10 isoform b) c4(30181) c5(30182, 30183, 30184, 30185, 30186, 30187) c6( ) c7(bL, ck, BI, T, am, aE, cc, qb, aW, aT, aT, hR, cy, ci, pw, aot, t, bkB, g, eJ); #c1(KLF10) c2(NP_005646) c3(Krueppel-like factor 10 isoform a) c4(158243) c5(158244, 158245, 158246, 158247, 158248, 158249, 158250) c6( ) c7(bL, ck, Bl, T, am, aE, cc, qb, aW, aT, aT, hR, cy, ci, pw, aot, t, bkB, g, eJ); #c1(KLF11) c2(NP_003588) c3(Krueppel-like factor Il isoform a) c4(139382, 139383) c5(139384, 139385, 139386, 139387, 139388, 139389, 139390, 139391, 139392) c6( ) c7(bL, dP, T, mh, aE, bWy, op, vA, fc, dq, aj, J); #c1(KLF11) c2(XP_005246236) c3(Krueppel-like factor Il isoform X1) c4( ) c5(266975, 266976, 266977, 266978, 266979, 266980, 266981, 266982) c6( ) c7(bL, dP, T, mh, aE, bWy, op, vA, fc, dq, aj, J); #c1(KLF12) c2(XP_011533211) c3(Krueppel-like factor 12 isoform X1) c4(329338, 329339) c5(329340, 329341, 329342, 329343, 329344, 329345, 329346, 329347, 329348, 329349, 329350, 329351, 329352, 329353, 329354, 329355, 329356) c6( ) c7(bL, T, W, ajP, aA, aF, oQ, y, lB, ae, ad); #c1(KLF13) c2(NP_057079) c3(Krueppel-like factor 13 isoform 1) c4(188847) c5(188848, 188849, 188850, 188851, 188852, 188853, 188854) c6( ) c7(bL, eZ, dP); #c1(KLF14) c2(NP_619638) c3(Krueppel-like factor 14) c4(236951) c5(236952, 236953, 236954, 236955, 236956, 236957, 236958, 236959) c6( ) c7(bL, sS, hk, Y, afQ, ff, dq, j, en); #c1(KLF15) c2(NP_054798) c3(Krueppel-like factor 15) c4( ) c5(177999, 178000, 178001, 178002, 178003, 178004) c6( ) c7(bL, dD, bf, tQ, hX, ea, dh, fc, h, rx, v, aS, K, eq, cd, us, ad, vQ); #c1(KLF15) c2(XP_011511045) c3(Krueppel-like factor 15 isoform X2) c4(301468) c5(301469, 301470) c6( ) c7(bL, dD, bf, tQ, hX, ea, dh, fc, h, rx, v, aS, K, eq, cd, us, ad, vQ); #c1(KLF16) c2(NP_114124) c3(Krueppel-like factor 16) c4(222258) c5(222259, 222260, 222261, 222262, 222263, 222264, 222265, 222266, 222267, 222268, 222269, 222270) c6( ) c7(bL); #c1(KLF2) c2(NP_057354) c3(Krueppel-like factor 2) c4(190305) c5(190306, 190307, 190308, 190309, 190310, 190311, 190312) c6( ) c7(bL, T, DM, dD, ea, e, al, cR, aj, j, eq, C, J, M, aT, h, rO, K, aa, sj, oV, DF, gk, ac, eM, at, aS, fD, ao); #c1(KLF3) c2(NP_057615) c3(Krueppel-like factor 3) c4(191782) c5(191783, 191784, 191785, 191786, 191787, 191788, 191789) c6( ) c7(bL, aC, c, e, bt, vQ); #c1(KLF4) c2(NP_004226) c3(Krueppel-like factor 4) c4(144431, 144432, 144433, 144434) c5(144435, 144436, 144437, 144438, 144439, 144440, 144441, 144442, 144443, 144444, 144445, 144446, 144447, 144448, 144449, 144450, 144451, 144452, 144453, 144454, 144455) c6(144456) c7(T, aC, tQ, ea, if, EQ, fd, bB, ci, x, aE, K, hu, pk, eL, Qe, Jl, aW, OZ, as, cm, ou, iA, U, dD, QO, wu, aw, ht, n, fr, qN, kH, eV, dL, bwj, bAF, kp, pm, bm, bD, aAE, B, bL, fM, dA, yk, eX, aA, cz, v, j, AO, q, eA, aT, rO, ae, bf, s, ev, r, en, iK, ck, sS, iM, kP, gX, Da, bt, Dd, jh, eq, ad, py, wm, d, je, ff, aF, bz, bF, es, bE, aS, adg, lC, X, BA); #c1(KLF5) c2(NP_001273747) c3(Krueppel-like factor 5 isoform 2) c4( ) c5(105755, 105756, 105757, 105758, 105759) c6( ) c7(bL, T, aD, e, ht, cz, bB, n, Gn, ci, q, ad, av, aE, rA, aT, dL, jj, ae, af, eL, s, aW, ff, r, en, t, bz, sE, W, eM, lT, bE, aS, m, GF, bt, Mj); #c1(KLF5) c2(NP_001721) c3(Krueppel-like factor 5 isoform 1) c4(120496, 120497, 120498) c5(120499, 120500, 120501, 120502, 120503, 120504, 120505, 120506, 120507, 120508, 120509, 120510, 120511, 120512) c6( ) c7(bL, T, aD, e, ht, cz, bB, n, Gn, ci, q, ad, av, aE, rA, aT, dL, jj, ae, af, eL, s, aW, ff, r, en, t, bz, sE, W, eM, lT, bE, aS, m, GF, bt, Mj); #c1(KLF6) c2(NP_001153597) c3(Krueppel-like factor 6 isoform C) c4(60693, 60694, 60695) c5(60696, 60697, 60698, 60699, 60700, 60701, 60702, 60703, 60704, 60705, 60706, 60707, 60708, 60709, 60710, 60711, 60712, 60713) c6( ) c7(bL, T, ul, al, jx, aA, fd, axk, aj, j, ci, uu, Fb, hF, AD, Fc, q, mC, J, ad, hX, eA, av, iu, ds, pX, aT, ab, bK, K, jj, he, ae, cp, Qe, d, BO, Ub, aW, Y, eH, fc, r, fe, aq, lo, kp, aF, g, as, eF, t, cM, fq, bz, cm, bF, gk, ac, eM, bP, dB, eD, Ei, hR, m, cP, gn, pK, bt, B); #c1(KLF7) c2(NP_001257871) c3(Krueppel-like factor 7 isoform 2) c4(95695, 95696) c5(95697, 95698, 95699, 95700, 95701, 95702, 95703, 95704, 95705, 95706, 95707, 95708) c6( ) c7(bL, aur, v); #c1(KLF7) c2(NP_001257872) c3(Krueppel-like factor 7 isoform 3) c4(95709) c5(95710, 95711, 95712, 95713, 95714, 95715, 95716, 95717, 95718, 95719, 95720, 95721) c6( ) c7(bL, aur, v); #c1(KLF7) c2(NP_001257873) c3(Krueppel-like factor 7 isoform 4) c4(95722) c5(95723, 95724, 95725, 95726, 95727, 95728) c6( ) c7(bL, aur, v); #c1(KLF7) c2(NP_003700) c3(Krueppel-like factor 7 isoform 1) c4(140257, 140258) c5(140259, 140260, 140261, 140262, 140263, 140264, 140265) c6( ) c7(bL, aur, v); #c1(KLF8) c2(XP_005262036) c3(Krueppel-like factor 8 isoform X1) c4(277720) c5(277721, 277722, 277723, 277724, 277725, 277726, 277727) c6( ) c7(bL, T, d, py, iA, aW, aT, aA, n, aF, fr, gn, m, nA, eA, t, B); #c1(KLF8) c2(XP_006724638) c3(Krueppel-like factor 8 isoform X2) c4(296154) c5(296155, 296156, 296157, 296158, 296159, 296160) c6( ) c7(bL, T, d, py, iA, aW, aT, aA, n, aF, fr, gn, m, nA, eA, t, B); #c1(KLF9) c2(NP_001197) c3(Krueppel-like factor 9) c4(83513) c5(83514, 83515, 83516, 83517, 83518, 83519) c6( ) c7(dP, T, am, d, AV, al, hL, hz, cl, PU, ae, B, ad); #c1(KLHDC1) c2(NP_751943) c3(kelch domain-containing protein 1) c4(248559) c5(248560, 248561, 248562, 248563, 248564, 248565) c6( ) c7(bL, y, A, j, cz); #c1(KLHDC2) c2(NP_055130) c3(kelch domain-containing protein 2) c4(179166) c5(179167, 179168, 179169, 179170, 179171, 179172) c6( ) c7(eM); #c1(KLHDC8A) c2(XP_006711492) c3(kelch domain-containing protein 8A isoform X1) c4(286018, 286019) c5(286020, 286021, 286022, 286023, 286024, 286025, 286026, 286027, 286028, 286029, 286030, 286031, 286032, 286033, 286034, 286035, 286036, 286037, 286038, 286039) c6( ) c7(bX); #c1(KLHDC8B) c2(NP_775817) c3(kelch domain-containing protein 8B) c4(250396) c5(250397, 250398, 250399, 250400, 250401, 250402, 250403) c6( ) c7(h, eV, ex, zL); #c1(KLHDC8B) c2(XP_005264995) c3(kelch domain-containing protein 88 isoform X3) c4(279324) c5( ) c6( ) c7(h, eV, ex, zL); #c1(KLHDC8B) c2(XP_005264997) c3(kelch domain-containing protein 8B isoform X5) c4( ) c5(279325, 279326, 279327, 279328, 279329) c6( ) c7(h, eV, ex, zL); #c1(KLHL1) c2(NP_001273654) c3(kelch-like protein 1 isoform 2) c4(105556) c5(105557, 105558, 105559, 105560, 105561, 105562) c6( ) c7(T, ha, jx, fr, ci, lu, q, ft, F, ib, aE, jL, aT, bc, ls, aW, Y, kK, hC, ff, lo, aF, en, hf, hJ, g, bz, eM, KB, m, hE, bt, B, hS); #c1(KLHL1) c2(NP_065917) c3(kelch-like protein 1 isoform 1) c4(206243) c5(206244, 206245, 206246, 206247, 206248, 206249, 206250) c6( ) c7(T, ha, jx, fr, ci, lu, q, ft, F, ib, aE, jL, aT, bc, ls, aW, Y, kK, hC, ff, lo, aF, en, hf, hJ, g, bz, eM, kB, m, hE, bt, B, hS); #c1(KLHL10) c2(NP_689680) c3(kelch-like protein 10) c4(243553, 243554) c5(243555, 243556, 243557, 243558, 243559, 243560, 243561, 243562, 243563, 243564, 243565, 243566) c6( ) c7(vH, ND, gU, ZE, bWz); #c1(KLHL12) c2(NP_001289980) c3(kelch-like protein 12 isoform 1) c4(114919, 114920) c5(114921, 114922, 114923, 114924, 114925, 114926, 114927, 114928, 114929, 114930, 114931, 114932) c6( ) c7(cz); #c1(KLHL12) c2(NP_067646) c3(kelch-like protein 12 isoform 2) c4(209210) c5(209211, 209212, 209213, 209214, 209215) c6( ) c7(cz); #c1(KLHL12) c2(XP_011508137) c3(kelch-like protein 12 isoform X1) c4( ) c5(298624, 298625) c6( ) c7(cz); #c1(KLHL12) c2(XP_011508138) c3(kelch-like protein 12 isoform X2) c4(298626) c5(298627, 298628, 298629, 298630, 298631, 298632) c6( ) c7(cz); #c1(KLHL2) c2(NP_001154993) c3(kelch-like protein 2 isoform 2) c4(61248) c5(61249, 61250, 61251, 61252, 61253, 61254) c6( ) c7(bWA, xX, d, eM, dB, atC, WP, Ws, Am, r, cz, K, aVg, lC, q, er); #c1(KLHL2) c2(NP_009177) c3(kelch-like protein 2 isoform 1) c4(171317, 171318) c5(171319, 171320, 171321, 171322, 171323, 171324, 171325, 171326, 171327, 171328, 171329, 171330) c6( ) c7(bWA, xX, d, eM, dB, atC, WP, Ws, Am, r, cz, K, aVg, lC, q, er); #c1(KLHL2) c2(XP_011529878) c3(kelch-like protein 2 isoform X4) c4( ) c5(325648) c6( ) c7(bWA, xX, d, eM, dB, atC, WP, Ws, Am, r, cz, K, aVg, lC, q, er); #c1(KLHL20) c2(NP_055273) c3(kelch-like protein 20) c4(180198, 180199) c5(180200, 180201, 180202, 180203, 180204, 180205, 180206, 180207, 180208, 180209, 180210, 180211) c6( ) c7(W); #c1(KLHL25) c2(NP_071925) c3(kelch-like protein 25) c4(212343, 212344) c5(212345, 212346, 212347, 212348, 212349, 212350, 212351, 212352, 212353, 212354, 212355, 212356, 212357, 212358, 212359, 212360) c6( ) c7(bA, Ea); #c1(KLHL26) c2(NP_060786) c3(kelch-like protein 26) c4(197802, 197803) c5(197804, 197805, 197806, 197807, 197808, 197809, 197810, 197811, 197812, 197813, 197814, 197815) c6( ) c7(Nc); #c1(KLHL29) c2(NP_443152) c3(kelch-like protein 29) c4(229663) c5(229664, 229665, 229666, 229667, 229668, 229669) c6( ) c7(fq, j); #c1(KLHL3) c2(NP_001244123) c3(kelch-like protein 3 isoform 2) c4( ) c5(91942, 91943, 91944, 91945, 91946, 91947) c6( ) c7(bWB, ahS, iT, cz); #c1(KLHL3) c2(NP_001244124) c3(kelch-like protein 3 isoform 3) c4( ) c5(91948, 91949, 91950, 91951, 91952, 91953, 91954, 91955, 91956, 91957) c6( ) c7(bWB, ahS, iT, cz); #c1(KLHL3) c2(NP_059111) c3(kelch-like protein 3 isoform 1) c4(193072, 193073, 193074, 193075, 193076) c5(193077, 193078, 193079, 193080, 193081, 193082, 193083, 193084, 193085, 193086, 193087, 193088, 193089, 193090) c6( ) c7(bWB, ahS, iT, cz); #c1(KLHL31) c2(NP_001003760) c3(kelch-like protein 31) c4(20075) c5(20076, 20077, 20078, 20079, 20080, 20081, 20082, 20083, 20084, 20085) c6( ) c7(cz); #c(KLHL35) c2(NP_001034637) c3(kelch-like protein 35) c4( ) c5(31946, 31947, 31948, 31949, 31950, 31951, 31952, 31953, 31954, 31955, 31956, 31957) c6( ) c7(fr, B); #c1(KLHL35) c2(XP_011543256) c3(kelch-like protein 35 isoform X1) c4(342057, 342058) c5( ) c6( ) c7(fr, B); #c1(KLHL40) c2(NP_689606) c3(kelch-like protein 40) c4(243245, 243246) c5(243247, 243248, 243249, 243250, 243251, 243252, 243253, 243254, 243255, 243256, 243257, 243258, 243259) c6( ) c7(Ag, Am, oj, bWC); #c1(KLHL41) c2(NP_006054) c3(kelch-like protein 41) c4(161513) c5(161514, 161515, 161516, 161517, 161518, 161519, 161520) c6( ) c7(bWQ, Am); #c1(KLHL42) c2(NP_065833) c3(kelch-like protein 42) c4(205745) c5(205746, 205747, 205748, 205749, 205750, 205751) c6( ) c7(bL, eU); #c1(KLHL5) c2(NP_001007076) c3(kelch-like protein 5 isoform 3) c4(22464) c5(22465, 22466, 22467, 22468, 22469, 22470, 22471, 22472, 22473, 22474, 22475) c6( ) c7(cu); #c1(KLHL5) c2(NP_001165125) c3(kelch-like protein 5 isoform 4) c4(67504) c5(67505, 67506, 67507, 67508, 67509, 67510) c6( ) c7(cu); #c1(KLHL5) c2(NP_057074) c3(kelch-like protein 5 isoform 1) c4(188806, 188807, 188808) c5(188809, 188810, 188811, 188812, 188813, 188814, 188815, 188816, 188817, 188818, 188819, 188820, 188821) c6( ) c7(cu); #c1(KLHL5) c2(NP_950240) c3(kelch-like protein 5 isoform 2) c4(260913) c5(260914, 260915, 260916, 260917, 260918, 260919) c6( ) c7(cu); #c1(KLHL5) c2(XP_005262712) c3(kelch-like protein 5 isoform X2) c4(278048) c5(278049, 278050, 278051, 278052, 278053, 278054, 278055) c6( ) c7(cu); #c1(KLHL5) c2(XP_011512001) c3(kelch-like protein 5 isoform X1) c4(302672, 302673) c5(302674, 302675, 302676, 302677, 302678, 302679, 302680, 302681, 302682, 302683, 302684, 302685) c6( ) c7(cu); #c1(KLHL6) c2(NP_569713) c3(kelch-like protein 6) c4(233820) c5(233821, 233822, 233823, 233824, 233825, 233826, 233827) c6( ) c7(cj); #c1(KLHL7) c2(NP_001026880) c3(kelch-like protein 7 isoform 1) c4(29792, 29793, 29794, 29795, 29796) c5(29797, 29798, 29799, 29800, 29801, 29802, 29803, 29804, 29805, 29806, 29807, 29808, 29809, 29810, 29811, 29812, 29813, 29814, 29815, 29816, 29817, 29818, 29819, 29820) c6( ) c7(cj, bWE, xX, lR, nw); #c1(KLHL7) c2(NP_001165899) c3(kelch-like protein 7 isoform 3) c4(67967) c5(67968, 67969, 67970, 67971, 67972, 67973) c6( ) c7(cj, bWE, xX, lR, nw); #c1(KLHL7) c2(NP_061334) c3(kelch-like protein 7 isoform 2) c4( ) c5(200109, 200110, 200111, 200112, 200113, 200114, 200115, 200116, 200117, 200118, 200119) c6( ) c7(cj, bWE, xX, lR, nw); #c1(KLHL7) c2(XP_006715816) c3(kelch-like protein 7 isoform X1) c4(289799) c5( ) c6( ) c7(cj, bWE, xX, lR, nw); #c1(KLHL7) c2(XP_006715818) c3(kelch-like protein 7 isoform X2) c4(289800, 289801) c5(289802, 289803, 289804, 289805, 289806, 289807, 289808, 289809, 289810, 289811, 289812, 289813, 289814, 289815, 289816, 289817) c6( ) c7(cj, bWE, xX, lR, nw); #c1(KLHL7) c2(XP_006715819) c3(kelch-like protein 7 isoform X3) c4( ) c5(289818) c6( ) c7(cj, bWE, xX, lR, nw); #c1(KLHL9) c2(NP_061335) c3(kelch-like protein 9) c4(200120, 200121) c5(200122, 200123, 200124, 200125, 200126, 200127, 200128, 200129, 200130, 200131, 200132, 200133) c6( ) c7(hk, Y); #c1(KLK1) c2(NP_002248) c3(kallikrein-1 preproprotein) c4(126933) c5(126934, 126935, 126936, 126937, 126938, 126939, 126940) c6( ) c7(bL, gx, T, bM, rG, eR, lT, e, uv, aw, cz, bB, aj, v, j, ci, fr, sl, il, aY, q, J, ad, ip, fU, cc, wG, sN, M, aT, cu, lG, cy, jy, K, bq, ae, dl, bWF, hm, vP, d, bf, LC, aW, cr, r, eZ, fe, kp, aB, aF, g, iV, eE, bUw, t, uC, pb, qa, ek, bLL, dR, ar, TA, tY, ac, bE, wO, go, X, bm, iS, hR, E, bWG, dy, m, y, gD, us); #c1(KLK1) c2(XP_011525244) c3(kallikrein-1 isoform X1) c4(319712) c5(319713, 319714, 319715, 319716, 319717) c6( ) c7(bL, gx, T, bM, rG, eR, lT, e, uv, aw, cz, bB, aj, v, j, ci, fr, sl, il, aY, q, J, ad, ip, fU, cc, wG, sN, M, aT, cU, lG, cy, jy, K, bq, ae, dl, bWF, hm, vP, d, bf, LC, aW, cr, r, eZ, fe, kp, aB, aF, g, iV, eE, bUw, t, uC, pb, qa, ek, bLL, dR, ar, TA, tY, ac, bE, wO, go, X, bm, iS, hR, E, bWG, dy, m, y, gD, us); #c1(KLK10) c2(NP_002767) c3(kallikrein-10 preproprotein) c4(131832) c5(131833, 131834, 131835, 131836, 131837, 131838, 131839, 131840, 131841, 131842, 131843, 131844, 131845, 131846, 131847) c6( ) c7(T, al, aA, ci, q, ad, hX, Db, aT, Dz, ae, qt, hT, aW, ff, cr, r, kp, aF, en, g, t, ML, gk, eM, wB, gX, m, B); #c1(KLK11) c2(NP_001129504) c3(kallikrein-Il isoform 1 precursor) c4( ) c5(51758, 51759, 51760, 51761, 51762, 51763, 51764, 51765) c6( ) c7(G, T, ib, s, eM, aW, cr, aT, aA, aF, fr, ci, m, t, ae, ad); #c1(KLK11) c2(NP_001161077) c3(kallikrein-Il isoform 3 precursor) c4( ) c5(65272, 65273, 65274, 65275, 65276) c6( ) c7(G, T, ib, s, eM, aW, cr, aT, aA, aF, fr, ci, m, t, ae, ad); #c1(KLK11) c2(NP_659196) c3(kallikrein-11 isoform 2) c4(239716, 239717) c5(239718, 239719, 239720, 239721, 239722, 239723, 239724, 239725, 239726, 239727, 239728, 239729) c6( ) c7(G, T, ib, s, eM, aW, cr, aT, aA, aF, fr, ci, m, t, ae, ad); #c1(KLK11) c2(XP_011524671) c3(kallikrein-Il isoform X2) c4(319177) c5(319178) c6( ) c7(G, T, ib, s, eM, aW, cr, aT, aA, aF, fr, ci, m, t, ae, ad); #c1(KLK12) c2(NP_062544) c3(kallikrein-12 isoform 1 precursor) c4(201901) c5(201902, 201903, 201904, 201905, 201906, 201907) c6( ) c7(T, aW, aA, aT, aF, g, as, pb, ad); #c1(KLK12) c2(NP_665901) c3(kallikrein-12 isoform 2 precursor) c4(241945) c5(241946, 241947, 241948, 241949, 241950, 241951, 241952, 241953, 241954, 241955, 241956) c6( ) c7(T, aW, aA, aT, aF, g, ae, pb, ad); #c1(KLK12) c2(NP_665902) c3(kallikrein-12 isoform 3 precursor) c4(241957) c5(241958, 241959, 241960, 241961, 241962) c6( ) c7(T, aW, aA, aT, aF, g, ae, pb, ad); #c1(KLK12) c2(XP_005259007) c3(kallikrein-12 isoform X2) c4(275916) c5(275917, 275918, 275919, 275920, 275921, 275922, 275923) c6( ) c7(T, aW, aA, aT, aF, g, ae, pb, ad); #c1(KLK13) c2(NP_056411) c3(kallikrein-13 precursor) c4(187462, 187463, 187464) c5(187465, 187466, 187467, 187468, 187469, 187470, 187471, 187472, 187473, 187474, 187475, 187476, 187477, 187478, 187479, 187480, 187481, 187482, 187483, 187484, 187485, 187486, 187487, 187488, 187489, 187490) c6( ) c7(av, T, eM, aW, Lf, cr, aT, aA, n, af, ci, m, bu, g, t, ae, ad); #c1(KLK14) c2(NP_071329) c3(kallikrein-14 preproprotein) c4(211008) c5(211009, 211010, 211011, 211012, 211013, 211014, 211015, 211016, 211017, 211018, 211019, 211020, 211021, 211022, 211023, 211024, 211025, 211026, 211027, 211028) c6( ) c7(T, xE, s, eM, bE, aW, ev, cr, aT, bB, Fb, m, g, bu, t, ae, ad); #c1(KLK15) c2(NP_001264010) c3(kallikrein-15 isoform 5 preproprotein) c4( ) c5(97439, 97440) c6( ) c7(T, aW, aT, n, Fb, PQ, m, g, t, ae, ad); #c1(KLK15) c2(NP_059979) c3(kallikrein-15 isoform 4 preproprotein) c4(193452) c5(193453, 193454, 193455, 193456, 193457, 193458, 193459, 193460, 193461, 193462) c6( ) c7(T, aW, aT, n, Fb, PQ, m, g, t, ae, ad); #c1(KLK15) c2(XP_011525389) c3(kallikrein-15 isoform X4) c4(319826) c5(319827, 319828, 319829, 319830, 319831, 319832) c6( ) c7(T, aW, aT, n, Fb, PQ, m, g, t, ae, ad); #c1(KLK15) c2(XP_011525390) c3(kallikrein-15 isoform X5) c4(319833, 319834) c5(319835, 319836, 319837, 319838, 319839, 319840, 319841, 319842, 319843, 319844) c6( ) c7(T, aW, aT, n, Fb, PQ, m, g, t, ae, ad); #c1(KLK2) c2(NP_001002231) c3(kallikrein-2 isoform 2 preproprotein) c4(19434) c5(19435, 19436, 19437, 19438, 19439, 19440) c6( ) c7(HQ, ae, T, dH, aW, aT, cz, n, aj, g, bz, ne, J, ad); #c1(KLK2) c2(NP_001243009) c3(kallikrein-2 isoform 3) c4(89535) c5(89536, 89537, 89538, 89539, 89540) c6( ) c7(HQ, ae, T, dH, aW, aT, cz, n, aj, g, bz, ne, J, ad); #c1(KLK2) c2(NP_005542) c3(kallikrein-2 isoform 1 preproprotein) c4(157255) c5(157256, 157257, 157258, 157259, 157260, 157261, 157262) c6( ) c7(HQ, ae, T, dH, aW, aT, cz, n, aj, g, bz, ne, J, ad); #c1(KLK3) c2(NP_001025218) c3(prostate-specific antigen isoform 3 preproprotein) c4(29503) c5(29504, 29505, 29506, 29507, 29508, 29509) c6( ) c7(gh, G, T, bWH, ci, aY, asr, gH, il, K, aeN, aa, li, UL, aW, dg, g, cm, Jr, cX, OD, m, Uw, WA, sw, ez, n, fr, eW, b, no, aow, is, cY, RT, AQ, hq, GU, dB, hk, dA, eR, aA, fF, ze, q, er, azn, aT, ae, af, ne, kl, zH, oV, Y, OS, r, eS, en, apx, t, UF, LS, bWl, id, rQ, Dd, iW, jN, Fb, ad, av, ji, Ub, ff, cr, eZ, aF, bF, pu, asq, ag, dX, i, iu, jr); #c1(KLK3) c2(NP_001025219) c3(prostate-specific antigen isoform 4 preproprotein) c4(29510, 29511) c5(29512, 29513, 29514, 29515, 29516, 29517, 29518, 29519, 29520, 29521, 29522, 29523, 29524, 29525, 29526, 29527) c6( ) c7(gh, G, T, bWH, ci, aY, asr, gH, il, K, aeN, aa, li, UL, aW, dg, g, cm, Jr, cX, OD, m, Uw, WA, sw, ez, n, fr, eW, b, no, aow, is, cY, RT, AQ, hq, GU, dB, hk, dA, eR, aA, fF, ze, q, er, azn, aT, ae, af, ne, kl, zH, oV, Y, OS, r, eS, en, apx, t, UF, LS, bWl, id, rQ, Dd, iW, jN, Fb, ad, av, ji, Ub, ff, cr, eZ, aF, bF, pu, asq, ag, dX, i, iu, jr); #c1(KLK3) c2(NP_001639) c3(prostate-specific antigen isoform 1 preproprotein) c4(119547, 119548) c5(119549, 119550, 119551, 119552, 119553, 119554, 119555, 119556, 119557, 119558, 119559, 119560) c6( ) c7(gh, G, T, bWH, ci, aY, asr, gH, il, K, aeN, aa, li, UL, aW, dg, g, cm, Jr, cX, OD, m, Uw, WA, sw, ez, n, fr, eW, b, no, aow, is, cY, RT, AQ, hq, GU, dB, hk, dA, eR, aA, fF, ze, q, er, azn, aT, ae, af, ne, kl, zH, oV, Y, OS, r, eS, en, apx, t, UF, LS, bWl, id, rQ, Dd, iW, jN, Fb, ad, av, ji, Ub, ff, cr, eZ, aF, bF, pu, asq, ag, dX, i, iu, jr); #c1(KLK4) c2(NP_004908) c3(kallikrein-4 isoform 1 preproprotein) c4(150704) c5(150705, 150706, 150707, 150708, 150709, 150710) c6( ) c7(T, eR, aw, cz, j, ci, uE, cd, q, mC, ad, BJ, aT, h, cy, K, ae, RT, s, aW, eZ, r, g, cl, wA, t, Ud, ek, uP, bWJ, ac, wO, hL, dy, m, fL, us); #c1(KLK4) c2(XP_011525848) c3(kallikrein-4 isoform X3) c4( ) c5(320264, 320265, 320266, 320267, 320268, 320269, 320270) c6( ) c7(T, eR, aw, cz, j, ci, uE, cd, q, mC, ad, BJ, aT, h, cy, K, ae, RT, s, aW, eZ, r, g, cl, wA, t, Ud, ek, uP, bWJ, ac, wO, hL, dy, m, fL, us); #c1(KLK4) c2(XP_011525849) c3(kallikrein-4 isoform X4) c4(320271) c5( ) c6( ) c7(T, eR, aw, cz, j, ci, uE, cd, q, mC, ad, BJ, aT, h, cy, K, ae, RT, s, aW, eZ, r, g, cl, wA, t, Ud, ek, uP, bWJ, ac, wO, hL, dy, m, fL, us); #c1(KLK5) c2(NP_036559) c3(kallikrein-5 preproprotein) c4(174385, 174386) c5(174387, 174388, 174389, 174390, 174391, 174392, 174393, 174394, 174395, 174396, 174397, 174398, 174399, 174400) c6( ) c7(gh, cT, T, aW, eL, ff, cr, aT, aEW, dL, m, en, eE, t, ae, LS, ad); #c1(KLK6) c2(NP_001012982) c3(kallikrein-6 isoform A preproprotein) c4(25384, 25385, 25386) c5(25387, 25388, 25389, 25390, 25391, 25392, 25393, 25394, 25395, 25396, 25397, 25398, 25399, 25400, 25401, 25402, 25403, 25404, 25405) c6( ) c7(T, Uc, dA, bWK, aA, n, bB, fr, ci, mF, V, q, ad, ip, ke, Db, aT, ae, af, d, s, aW, cr, r, aEW, kp, aF, g, cl, eE, t, wA, bE, go, iS, m); #c1(KLK6) c2(NP_001012983) c3(kallikrein-6 isoform B) c4( ) c5(25406, 25407, 25408, 25409, 25410, 25411, 25412, 25413, 25414) c6( ) c7(T, Uc, dA, bWK, aA, n, bB, fr, ci, mF, V, q, ad, ip, ke, Db, aT, ae, af, d, s, aW, cr, r, aEW, kp, aF, g, cl, eE, t, wA, bE, go, iS, m); #c1(KLK7) c2(NP_001193982) c3(kallikrein-7 isoform 2) c4( ) c5(83419, 83420, 83421, 83422, 83423, 83424, 83425) c6(83426, 83427) c7(T, aC, Uc, bB, fr, ci, q, ad, aE, apJ, aT, lQ, ae, s, aW, cr, r, dN, aEW, bSc, g, eE, t, bE, go, m); #c1(KLK7) c2(NP_001230055) c3(kallikrein-7 isoform 3) c4( ) c5(85860) c6( ) c7(T, aC, Uc, bB, fr, ci, q, ad, aE, apJ, aT, lQ, ae, s, aW, cr, r, dN, aEW, bSc, g, eE, t, bE, go, m); #c1(KLK7) c2(NP_005037) c3(kallikrein-7 isoform 1 preproprotein) c4(152354, 152355) c5(152356, 152357, 152358, 152359, 152360, 152361, 152362, 152363, 152364, 152365, 152366, 152367, 152368, 152369) c6(152370, 152371, 152372, 152373) c7(T, aC, Uc, bB, fr, ci, q, ad, aE, apJ, aT, lQ, ae, s, aW, cr, r, dN, aEW, bSc, g, eE, t, bE, go, m); #c1(KLK8) c2(NP_009127) c3(kallikrein-8 isoform 1 preproprotein) c4(170941) c5(170942, 170943, 170944, 170945, 170946, 170947, 170948, 170949, 170950, 170951) c6( ) c7(av, T, Ew, eM, aW, if, VB, aT, dN, n, he, bk, K, m, qN, g, t, ik, bz, ad); #c1(KLK8) c2(NP_653088) c3(kallikrein-8 isoform 2 precursor) c4(238851) c5(238852, 238853, 238854, 238855, 238856, 238857) c6( ) c7(av, T, Ew, eM, aW, if, VB, aT, dN, n, he, bK, K, m, qN, g, t, iK, bz, ad); #c1(KLK8) c2(NP_653089) c3(kallikrein-8 isoform 3 precursor) c4(238858) c5(238859, 238860, 238861, 238862, 238863, 238864, 238865) c6( ) c7(av, T, Ew, eM, aW, if, VB, aT, dN, n, he, bK, K, m, qN, g, t, ik, bz, ad); #c1(KLK8) c2(NP_653090) c3(kallikrein-8 isoform 4) c4(238866) c5(238867, 238868, 238869, 238870, 238871) c6( ) c7(av, T, Ew, eM, aW, if, VB, aT, dN, n, he, bK, K, m, qN, g, t, iK, bz, ad); #c1(KLK9) c2(NP_036447) c3(kallikrein-9 precursor) c4(173931) c5(173932, 173933, 173934, 173935, 173936, 173937, 173938, 173939, 173940, 173941, 173942, 173943, 173944, 173945) c6( ) c7(T, aW, aT, m, t, ad); #c1(KLKB1) c2(NP_000883) c3(plasma kallikrein preproprotein) c4(16799, 16800) c5(16801, 16802, 16803, 16804, 16805, 16806, 16807, 16808, 16809, 16810, 16811, 16812) c6( ) c7(P, bM, T, eR, al, by, aA, cz, C, aY, bCx, ad, bnC, aT, ae, Zm, aW, fO, eZ, fe, af, Ci, qa, RJ, bvc, arO); #c1(KLLN) c2(NP_001119521) c3(killin) c4(44054) c5(44055, 44056, 44057, 44058, 44059, 44060) c6( ) c7(es, T, hd, nM, qw, iA, nB, aW, go, aT, bWM, bj, fr, ci, ae, ad, bWL); #c1(KLRB1) c2(NP_002249) c3(killer cell lectin-like receptor subfamily B member 1) c4(126941) c5(126942, 126943, 126944, 126945, 126946, 126947) c6( ) c7(wj, dA, ds, Y, jx, eb, h, lo, wV, ci, yH, hE, V, bWN, y, aDH, hX); #c1(KLRC1) c2(NP_998822) c3(NKG2-A/NKG2-B type II integral membrane protein isoform NKG2-B) c4(266035) c5(266036, 266037, 266038, 266039, 266040, 266041, 266042, 266043, 266044, 266045) c6( ) c7(T, fR, jx, jh, qj, jE, hX, qC, dP, F, ds, h, rB, hH, Y, eZ, cl, ck, hL, fL, y, pC); #c1(KLRC1) c2(NP_998823) c3(NKG2-A/NKG2-B type 11 integral membrane protein isoform NKG2-A) c4(266046) c5(266047, 266048, 266049, 266050, 266051, 266052, 266053, 266054, 266055, 266056) c6( ) c7(T, fR, jx, jh, qj, jE, hX, qC, dP, F, ds, h, rB, hH, Y, eZ, cl, ck, hL, fL, y, pC); #c1(KLRC2) c2(NP_002251) c3(NKG2-C type 11 integral membrane protein) c4(126948, 126949, 126950) c5(126951, 126952, 126953, 126954, 126955, 126956, 126957, 126958, 126959, 126960, 126961, 126962, 126963, 126964, 126965, 126966, 126967, 126968) c6( ) c7(T, hl, dc, cr, eZ, h, aSu, fL, hH, V, bWN, y); #c1(KLRC4) c2(NP_038459) c3(NKG2-F type II integral membrane protein) c4(176490, 176491) c5(176492, 176493, 176494, 176495, 176496, 176497, 176498, 176499, 176500, 176501, 176502) c6( ) c7(hH, T, jE, cj); #c1(KLRC4-KLRK1) c2(NP_001186734) c3(NKG2-D type II integral membrane protein) c4(78449, 78450) c5(78451, 78452, 78453, 78454, 78455, 78456, 78457, 78458, 78459, 78460, 78461, 78462, 78463, 78464, 78465, 78466, 78467) c6( ) c7(oO, uB, T, dD, eR, ea, if, iW, eX, oA, aA, bB, n, aj, dG, eq, ci, qN, wV, fd, aY, V, hF, q, amW, J, ad, hX, eA, wD, jz, blC, fh, oz, ds, h, eV, hn, K, dQ, ae, cY, buh, d, oV, Y, ff, eH, hl, eZ, r, dq, lo, gE, hN, aF, en, jh, eF, t, cm, bz, ik, bw, LB, ck, bF, eU, iT, nB, aWZ, bE, ig, atM, cj, at, dW, dy, m, y, bt, Pr, B); #c1(KLRD1) c2(NP_001107868) c3(natural killer cells antigen CD94 isoform 1) c4(43061) c5(43062, 43063, 43064, 43065, 43066, 43067, 43068, 43069, 43070, 43071, 43072, 43073, 43074, 43075, 43076, 43077) c6( ) c7(dP, T, y, F, A, fR, jx, eZ, jh, h, fL, jE, hH, VB, iP); #c1(KLRD1) c2(NP_031360) c3(natural killer cells antigen CD94 isoform 2) c4(171938) c5(171939, 171940, 171941, 171942, 171943, 171944, 171945, 171946, 171947, 171948, 171949, 171950) c6( ) c7(dP, T, y, F, A, fR, jx, eZ, jh, h, fL, jE, hH, VB, iP); #c1(KLRD1) c2(XP_006719129) c3(natural killer cells antigen CD94 isoform X5) c4(292212) c5( ) c6( ) c7(dP, T, y, F, A, fR, jx, eZ, jh, h, fL, jE, hH, VB, iP); #c1(KLRD1) c2(XP_006719130) c3(natural killer cells antigen CD94 isoform X7) c4(292213) c5(292214, 292215, 292216, 292217, 292218, 292219) c6( ) c7(dP, T, y, F, A, fR, jx, eZ, jh, h, fL, jE, hH, VB, iP); #c1(KLRD1) c2(XP_011518949) c3(natural killer cells antigen CD94 isoform X1) c4(313391) c5( ) c6( ) c7(dP, T, y, F, A, fR, jx, eZ, jh, h, fL, jE, hH, VB, iP); #c1(KLRG1) c2(NP_005801) c3(killer cell lectin-like receptor subfamily G member 1) c4(159574, 159575) c5(159576, 159577, 159578, 159579, 159580, 159581, 159582, 159583, 159584, 159585, 159586, 159587) c6( ) c7(bL, A, fR, ds, jx, eZ, cj, lo, dG, aj, ci, jM, J, yF, hX); #c1(KLRG2) c2(NP_940910) c3(killer cell lectin-like receptor subfamily G member 2) c4(260103) c5(260104, 260105, 260106, 260107, 260108, 260109) c6( ) c7(ae, ad); #c1(KLRG2) c2(XP_005250368) c3(killer cell lectin-like receptor subfamily G member 2 isoform X3) c4( ) c5(270275, 270276, 270277, 270278, 270279, 270280, 270281, 270282) c6( ) c7(ae, ad); #c1(KLRG2) c2(XP_011514443) c3(killer cell lectin-like receptor subfamily G member 2 isoform X2) c4(307133) c5( ) c6( ) c7(ae, ad); #c1(KMO) c2(NP_003670) c3(kynurenine 3-monooxygenase) c4(140081, 140082) c5(140083, 140084, 140085, 140086, 140087) c6( ) c7(x, ou, ft, rR, fJ, bO, bK, k, V); #c1(KMO) c2(XP_005273394) c3(kynurenine 3-monooxygenase isoform X1) c4(284556, 284557) c5(284558, 284559, 284560, 284561, 284562, 284563, 284564, 284565, 284566, 284567, 284568, 284569, 284570) c6( ) c7(x, ou, ft, rR, fJ, bO, bk, k, V); #c1(KMO) c2(XP_005273395) c3(kynurenine 3-monooxygenase isoform X2) c4( ) c5(284571, 284572, 284573, 284574, 284575, 284576) c6( ) c7(x, ou, ft, rR, fJ, bO, bK, k, V); #c1(KMT2A) c2(NP_001184033) c3(histone-lysine N-methyltransferase ZA isoform 1 precursor) c4(75285) c5(75286, 75287, 75288, 75289, 75290, 75291) c6( ) c7(T, ea, if, e, qx, fd, bB, ip, x, OB, pp, aEX, K, pk, eG, aW, g, Fu, aKj, cm, bw, ou, eM, iT, cj, k, ajJ, dD, aw, bWO, n, oY, fr, br, b, cq, ft, Bj, KH, h, hn, dL, bWP, B, fM, aA, hy, V, q, er, eA, aT, cy, dz, jj, ae, eu, iU, iZ, oV, Y, r, gm, hf, bu, f, ck, wR, dy, oX, bt, yv, id, DA, bs, jh, eq, hF, ad, hX, fx, iY, lQ, pq, iL, gj, BN, gK, dq, gE, bzD, aF, eB, aNL, i, y, kS, ne, jr); #c1(KMT2A) c2(NP_005924) c3(histone-lysine N-methyltransferase 2A isoform 2 precursor) c4(160454, 160455, 160456) c5(160457, 160458, 160459, 160460, 160461, 160462, 160463, 160464, 160465, 160466, 160467, 160468, 160469, 160470, 160471, 160472, 160473, 160474, 160475, 160476, 160477) c6( ) c7(T, ea, if, e, qx, fd, bB, ip, x, OB, pp, aEX, K, pk, eG, aW, g, Fu, aKj, cm, bw, ou, eM, iT, cj, k, ajJ, dD, aw, bWO, n, oY, fr, br, b, cq, ft, Bj, KH, h, hn, dL, bWP, B, fM, aA, hy, V, q, er, eA, aT, cy, dz, jj, ae, eu, iU, iZ, oV, Y, r, gm, hf, bu, f, ck, wR, dy, oX, bt, yv, id, DA, bs, jh, eq, hF, ad, hX, fx, iY, lQ, pq, iL, gj, BN, gK, dq, gE, bzD, aF, eB, aNL, i, y, kS, ne, jr); #c1(KMT2A) c2(XP_006718902) c3(histone-lysine N-methyltransferase 2A isoform X6) c4( ) c5(292045, 292046, 292047, 292048, 292049, 292050) c6( ) c7(T, ea, if, e, qx, fd, bB, ip, x, oB, pp, aEX, K, pk, eG, aW, g, Fu, aKj, cm, bw, ou, eM, iT, cj, k, ajJ, dD, aw, bWO, n, oY, fr, br, b, cq, ft, Bj, KH, h, hn, dL, bWP, B, fM, aA, hy, V, q, er, eA, aT, cy, dz, jj, ae, eu, iU, iZ, oV, Y, r, gm, hf, bu, f, ck, wR, dy, oX, bt, yv, id, DA, bs, jh, eq, hF, ad, hX, fx, iY, lQ, pq, iL, gj, BN, gK, dq, gE, bzD, aF, eB, aNL, i, y, kS, ne, jr); #c1(KMT2A) c2(XP_011541131) c3(histone-lysine N-methyltransferase 2A isoform X1) c4(339655) c5(339656, 339657, 339658, 339659, 339660, 339661) c6( ) c7(T, ea, if, e, qx, fd, bB, ip, x, oB, pp, aEX, K, pk, eG, aW, g, Fu, akj, cm, bw, ou, eM, iT, cj, k, ajJ, dD, aw, bWO, n, oY, fr, br, b, cq, ft, Bj, KH, h, hn, dL, bWP, B, fM, aA, hy, V, q, er, eA, aT, cy, dz, jj, ae, eu, iU, iZ, oV, Y, r, gm, hf, bu, f, ck, wR, dy, oX, bt, yv, id, DA, bs, jh, eq, hF, ad, hX, fx, iY, lQ, pq, iL, gj, BN, gK, dq, gE, bzD, aF, eB, aNL, i, y, kS, ne, jr); #c1(KMT2A) c2(XP_011541132) c3(histone-lysine N-methyltransferase 2A isoform X2) c4(339662) c5( ) c6( ) c7(T, ea, if, e, qx, fd, bB, ip, x, oB, pp, aEX, K, pk, eG, aW, g, Fu, akj, cm, bw, ou, eM, iT, cj, k, ajJ, dD, aw, bWO, n, oY, fr, br, b, cq, ft, Bj, KH, h, hn, dL, bWP, B, fM, aA, hy, V, q, er, eA, aT, cy, dz, jj, ae, eu, iU, iZ, oV, Y, r, gm, hf, bu, f, ck, wR, dy, oX, bt, yv, id, DA, bs, jh, eq, hF, ad, hX, fX, iY, lQ, pq, iL, gj, BN, gK, dq, gE, bzD, aF, eB, aNL, i, y, kS, ne, jr); #c1(KMT2A) c2(XP_011541133) c3(histone-lysine N-methyltransferase 2A isoform X3) c4( ) c5(339663, 339664, 339665, 339666, 339667, 339668) c6( ) c7(T, ea, if, e, qx, fd, bB, ip, x, oB, pp, aEX, K, pk, eG, aW, g, Fu, aKj, cm, bw, ou, eM, iT, cj, k, ajJ, dD, aw, bWO, n, oY, fr, br, b, cq, ft, Bj, KH, h, hn, dL, bWP, B, fM, aA, hy, V, q, er, eA, aT, cy, dz, jj, ae, eu, iU, iZ, oV, Y, r, gm, hf, bu, f, ck, wR, dy, oX, bt, yv, id, DA, bs, jh, eq, hF, ad, hX, fx, iY, lQ, pq, iL, gj, BN, gK, dq, gE, bzD, aF, eB, aNL, i, y, kS, ne, jr); #c1(KMT2A) c2(XP_011541135) c3(histone-lysine N-methyltransferase 2A isoform X5) c4(339669) c5( ) c6( ) c7(T, ea, if, e, qx, fd, bB, ip, x, oB, pp, aEX, K, pk, eG, aW, g, Fu, aKj, cm, bw, ou, eM, iT, cj, k, ajJ, dD, aw, bWO, n, oY, fr, br, b, cq, ft, Bj, kH, h, hn, dL, bWP, B, fM, aA, hy, V, q, er, eA, aT, cy, dz, jj, ae, eu, iU, iZ, oV, Y, r, gm, hf, bu, f, ck, wR, dy, oX, bt, yv, id, oA, bs, jh, eq, hF, ad, hX, fX, iY, lQ, pq, iL, gj, BN, gK, dq, gE, bzD, aF, eB, aNL, i, y, kS, ne, jr); #c1(KMT2B) c2(NP_055542) c3(histone-lysine N-methyltransferase 2B) c4(181847) c5(181848) c6( ) c7(T, fM, d, aE, py, aW, ff, jh, aT, aq, dy, K, nA, bX, en, bUh, B, ad, hX); #c1(KMT2B) c2(XP_011525863) c3(histone-lysine N-methyltransferase 2B isoform X1) c4( ) c5(320288, 320289, 320290, 320291, 320292, 320293, 320294, 320295, 320296, 320297) c6( ) c7(T, fM, d, aE, py, aW, ff, jh, aT, aq, dy, K, nA, bX, en, bUh, B, ad, hX); #c1(KMT2B) c2(XP_011525864) c3(histone-lysine N-methyltransferase 2B isoform X2) c4( ) c5(320298) c6( ) c7(T, fM, d, aE, py, aW, ff, jh, aT, aq, dy, K, nA, bX, en, bUh, B, ad, hX); #c1(KMT2B) c2(XP_011525865) c3(histone-lysine N-methyltransferase 2B isoform X3) c4(320299) c5(320300, 320301, 320302, 320303, 320304, 320305, 320306, 320307) c6( ) c7(T, fM, d, aE, py, aW, ff, jh, aT, aq, dy, K, nA, bX, en, bUh, B, ad, hX); #c1(KMT2C) c2(NP_733751) c3(histone-lysine N-methyltransferase 2C) c4(246941) c5(246942, 246943, 246944, 246945, 246946, 246947) c6( ) c7(T, id, aA, q, jz, qK, aT, hn, dL, he, aW, ff, r, aq, aF, en, g, hJ, ck, bcd, bt, it, B); #c1(KMT2C) c2(XP_005250082) c3(histone-lysine N-methyltransferase 2C isoform X1) c4( ) c5(270101, 270102, 270103, 270104, 270105) c6( ) c7(T, id, aA, q, jz, qK, aT, hn, dL, he, aW, ff, r, aq, aF, en, g, hJ, ck, bcd, bt, it, B); #c1(KMT2C) c2(XP_005250085) c3(histone-lysine N-methyltransferase 2C isoform X5) c4( ) c5(270106) c6( ) c7(T, id, aA, q, jz, qK, aT, hn, dL, he, aW, ff, r, aq, aF, en, g, hJ, ck, bed, bt, it, B); #c1(KMT2C) c2(XP_006716140) c3(histone-lysine N-methyltransferase 2C isoform X4) c4( ) c5(290091) c6( ) c7(T, id, aA, q, jz, qK, aT, hn, dL, he, aW, ff, r, aq, aF, en, g, hJ, ck, bcd, bt, it, B); #c1(KMT2C) c2(XP_006716141) c3(histone-lysine N-methyltransferase 2C isoform X7) c4(290092) c5(290093, 290094, 290095, 290096, 290097, 290098) c6( ) c7(T, id, aA, q, jz, qK, aT, hn, dL, he, aW, ff, r, aq, aF, en, g, hJ, ck, bed, bt, it, B); #c1(KMT2C) c2(XP_011514752) c3(histone-lysine N-methyltransferase 2C isoform X6) c4(307396) c5(307397, 307398, 307399, 307400, 307401, 307402) c6( ) c7(T, id, aA, q, jz, qK, aT, hn, dL, he, aW, ff, r, aq, aF, en, g, hJ, ck, bed, bt, it, B); #c1(KMT2C) c2(XP_011514757) c3(histone-lysine N-methyltransferase 2C isoform X14) c4( ) c5(307403) c6( ) c7(T, id, aA, q, jz, qK, aT, hn, dL, he, aW, ff, r, aq, aF, en, g, hJ, ck, bcd, bt, it, B); #c1(KMT2C) c2(XP_011514758) c3(histone-lysine N-methyltransferase 2C isoform X15) c4(307404) c5( ) c6( ) c7(T, id, aA, q, jz, qK, aT, hn, dL, he, aW, ff, r, aq, aF, en, g, hJ, ck, bed, bt, it, B); #c1(KMT2D) c2(XP_005269219) c3(histone-lysine N-methyltransferase 2D isoform X1) c4(282486) c5(282487, 282488, 282489, 282490, 282491, 282492) c6( ) c7(T, gU, fM, lP, jh, fd, aj, nA, bX, J, ad, hX, x, jz, py, aE, aT, K, jf, A, d, aW, oV, ff, aq, en, bUh, KM, ky, bE, mG, vy, dy, k, it, B); #c1(KMT2D) c2(XP_006719679) c3(histone-lysine N-methyltransferase 2D isoform X10) c4(292881) c5( ) c6( ) c7(T, gU, fM, lP, jh, fd, aj, nA, bX, J, ad, hX, x, jz, py, aE, aT, K, jf, A, d, aW, oV, ff, aq, en, bUh, KM, ky, bE, mG, vy, dy, k, it, B); #c1(KMT2D) c2(XP_011537072) c3(histone-lysine N-methyltransferase 2D isoform X2) c4( ) c5(334852, 334853, 334854, 334855, 334856, 334857) c6( ) c7(T, gU, fM, lP, jh, fd, aj, nA, bX, J, ad, hX, x, jz, py, aE, aT, K, jf, A, d, aW, oV, ff, aq, en, bUh, KM, ky, bE, mG, vy, dy, k, it, B); #c1(KMT2D) c2(XP_011537078) c3(histone-lysine N-methyltransferase 2D isoform X9) c4( ) c5(334858, 334859, 334860) c6( ) c7(T, gU, fM, lP, jh, fd, aj, nA, bX, J, ad, hX, x, jz, py, aE, aT, K, jf, A, d, aW, oV, ff, aq, en, bUh, KM, ky, bE, mG, vy, dy, k, it, B); #c1(KMT2E) c2(NP_061152) c3(histone-lysine N-methyltransferase 2E) c4(199561, 199562, 199563, 199564, 199565) c5(199566, 199567, 199568, 199569, 199570, 199571, 199572, 199573, 199574, 199575, 199576, 199577, 199578, 199579, 199580, 199581, 199582, 199583, 199584, 199585, 199586, 199587, 199588, 199589, 199590, 199591, 199592, 199593, 199594, 199595, 199596, 199597, 199598, 199599) c6( ) c7(ck, T, dD, iT, ea, if, eq, bt, fq); #c1(KMT2E) c2(XP_006716112) c3(histone-lysine N-methyltransferase 2E isoform X2) c4(290050, 290051, 290052, 290053, 290054) c5(290055, 290056, 290057, 290058, 290059, 290060, 290061, 290062, 290063, 290064, 290065, 290066, 290067, 290068, 290069, 290070, 290071, 290072, 290073, 290074, 290075, 290076, 290077, 290078, 290079, 290080, 290081, 290082, 290083, 290084, 290085) c6( ) c7(ck, T, dD, iT, ea, if, eq, bt, fq); #c1(KNG1) c2(NP_000884) c3(kininogen-1 isoform 2 precursor) c4(16813, 16814) c5(16815, 16816, 16817, 16818, 16819, 16820, 16821, 16822, 16823, 16824, 16825, 16826) c6( ) c7(bL, bM, T, dD, aoT, Wh, eR, ea, e, bWQ, aw, cz, aj, v, j, eq, C, pM, aY, cd, J, ad, eA, hd, F, cc, vh, Bc, cU, dL, K, Fg, eL, af, UO, zQ, bpL, bWS, fO, mQ, eZ, fQ, fe, zC, mo, iV, t, bWR, bz, cm, qa, ik, uP, ua, dB, RJ, at, E, dy, m, hE, gq); #c1(KNG1) c2(NP_001095886) c3(kininogen-1 isoform 1 precursor) c4(40892) c5(40893, 40894, 40895, 40896, 40897, 40898) c6( ) c7(bL, bM, T, dD, aoT, Wh, eR, ea, e, bWQ, aw, cz, aj, v, j, eq, C, pM, aY, cd, J, ad, eA, hd, F, cc, vh, Bc, cU, dL, K, Fg, eL, af, UO, zQ, bpL, bWS, fO, mQ, eZ, fQ, fe, zC, mo, iV, t, bWR, bz, cm, qa, ik, uP, ua, dB, RJ, at, E, dy, m, hE, gq); #c1(KNG1) c2(NP_001159923) c3(kininogen-1 isoform 3 precursor) c4(64986) c5(64987, 64988, 64989, 64990, 64991, 64992) c6( ) c7(bL, bM, T, dD, aoT, Wh, eR, ea, e, bWQ, aw, cz, aj, v, j, eq, C, pM, aY, cd, J, ad, eA, hd, F, cc, vh, Bc, c, dL, K, Fg, eL, af, UO, zQ, bpL, bWS, fO, mQ, eZ, fQ, fe, zC, mo, iV, t, bWR, bz, cm, qa, ik, uP, ua, dB, RJ, at, E, dy, m, hE, gq); #c1(KNSTRN) c2(NP_001136233) c3(small kinetochore-associated protein isoform b) c4(54528) c5(54529, 54530, 54531, 54532, 54533, 54534) c6( ) c7(T, bLl, eR, ff, cz, en, adg, aOo); #c1(KNSTRN) c2(NP_001136234) c3(small kinetochore-associated protein isoform c) c4(54535) c5(54536, 54537, 54538, 54539, 54540, 54541) c6( ) c7(T, bLl, eR, ff, cz, en, adg, aOo); #c1(KNSTRN) c2(NP_150628) c3(small kinetochore-associated protein isoform a) c4(228143) c5(228144, 228145, 228146, 228147, 228148, 228149) c6( ) c7(T, bLl, eR, ff, cz, en, adg, aOo); #c1(KNTC1) c2(NP_055523) c3(kinetochore-associated protein 1) c4(181746, 181747) c5(181748, 181749, 181750, 181751, 181752, 181753, 181754) c6( ) c7(h, TH, cF, QC, hj); #c1(KNTC1) c2(XP_011537330) c3(kinetochore-associated protein 1 isoform X3) c4( ) c5(335039, 335040, 335041, 335042, 335043) c6( ) c7(h, TH, cF, QC, hj); #c1(KPNA1) c2(NP_002255) c3(importin subunit alpha-5) c4(126983, 126984, 126985, 126986) c5(126987, 126988, 126989, 126990, 126991, 126992, 126993, 126994, 126995, 126996, 126997, 126998, 126999, 127000, 127001, 127002, 127003, 127004, 127005, 127006, 127007, 127008, 127009, 127010) c6( ) c7(y, ds, rR); #c1(KPNA2) c2(XP_011523085) c3(importin subunit alpha-1 isoform X1) c4(317583, 317584) c5(317585, 317586, 317587, 317588, 317589, 317590, 317591, 317592, 317593, 317594, 317595, 317596, 317597, 317598, 317599, 317600, 317601, 317602, 317603) c6( ) c7(T, fR, aA, n, ci, ad, aT, h, dL, ey, ae, eL, d, aW, Lh, aq, aF, as, iR, t, bz, ov, bP, eM, AH, m, KY, B); #c1(KPNA3) c2(NP_002258) c3(importin subunit alpha-4) c4(127022) c5(127023, 127024, 127025, 127026, 127027, 127028) c6( ) c7(ds, gv); #c1(KPNA4) c2(NP_002259) c3(importin subunit alpha-3) c4(127029) c5(127030, 127031, 127032, 127033, 127034, 127035) c6( ) c7(ds, ka, ki); #c1(KPNA6) c2(XP_011539462) c3(importin subunit alpha-7 isoform X2) c4( ) c5(337656, 337657, 337658, 337659, 337660, 337661, 337662, 337663, 337664, 337665, 337666, 337667, 337668, 337669, 337670, 337671) c6( ) c7(T, aE, cC); #c1(KPNA7) c2(NP_001139187) c3 (importin subunit alpha-8) c4( ) c5(58885, 58886, 58887, 58888, 58889) c6( ) c7(T, aE, cC); #c1(KPNA7) c2(XP_011514519) c3(importin subunit alpha-8 isoform X4) c4(307212) c5(307213) c6( ) c7(T, aE, cC); #c1(KPNB1) c2(NP_001263382) c3(importin subunit beta-1 isoform 2) c4( ) c5(97213, 97214, 97215, 97216, 97217, 97218, 97219, 97220, 97221, 97222, 97223, 97224, 97225, 97226) c6( ) c7(h, hP, AH, fR, aT); #c1(KPNB1) c2(NP_002256) c3(importin subunit beta-1 isoform 1) c4(127011, 127012, 127013) c5(127014, 127015, 127016, 127017, 127018, 127019, 127020, 127021) c6( ) c7(h, hP, AH, fR, aT); #c1(KPRP) c2(NP_001020402) c3(keratinocyte proline-rich protein) c4(28717, 28718, 28719) c5(28720, 28721, 28722, 28723, 28724, 28725, 28726, 28727, 28728, 28729, 28730, 28731) c6( ) c7(Xk); #c1(KPTN) c2(NP_001278225) c3(kaptin isoform 2) c4( ) c5(109517, 109518, 109519, 109520, 109521) c6( ) c7(bWT); #c1(KPTN) c2(NP_008990) c3(kaptin isoform 1) c4(170111, (70112) c5(170113, 170114, 170115, 170116, 170117, 170118, 170119) c6( ) c7(bWT); #c1(KPTN) c2(XP_011524703) c3(kaptin isoform X3) c4( ) c5(319192) c6( ) c7(bWT); #c1(KRAS) c2(NP_004976) c3(GTPase KRas isoform b) c4(151556) c5(151557, 151558, 151559, 151560, 151561, 151562) c6( ) c7(atT, T, aC, aEn, bkx, pf, yS, anl, if, al, fd, bB, abD, aub, ci, BWV, aJM, ip, bv, x, aE, il, pX, aeO, Qk, auB, iv, auf, K, hu, MO, eL, cp, YL, LQ, xQ, iE, aW, bWY, Ls, hl, QB, nY, Qq, Qv, Qm, kt, NR, g, hJ, as, ahv, cm, Qt, zV, mK, Fu, qJ, bw, eM, nB, AM, iT, vA, PV, k, m, YK, iu, wu, sw, zX, ht, NT, n, awr, kv, bWU, fr, qN, bao, br, atW, Bl, jz, hw, Tf, gd, h, auI, yT, hn, dL, auS, bWX, hB, bXd, aEL, Yl, bXa, auM, PX, aJL, blP, ge, fe, aq, agF, kp, po, YF, zZ, cM, Yr, Zj, op, ar, iO, Qy, bP, dB, Qu, Vy, ayS, Tk, hL, bcd, gw, at, nl, qF, bkE, bD, Te, Qz, it, B, zl, aJJ, dA, eR, jp, WT, jx, aA, bWZ, hy, v, bWW, hz, yO, AO, pd, auG, q, er, eA, eJ, av, hW, nX, BJ, qK, aT, bXb, Tj, atZ, ae, ne, s, vT, NE, oV, Y, ev, Or, BA, il, r, aMA, aJl, lo, en, bu, cl, LV, t, apx, ik, OC, ck, ov, nM, kP, Dy, auz, AH, gX, WU, anD, bsT, aws, zu, bt, vi, gV, hl, Mb, id, iW, aEh, atS, bXc, jh, auq, Fb, fu, ad, afb, bkO, qC, Jm, dP, hd, F, ib, Rq, Db, yt, vJ, NS, Yo, io, Ti, vc, hP, wm, YQ, fb, pq, Yj, auY, BN, BO, d, hG, qt, bXe, ff, eZ, Kf, aF, iV, bz, Hp, bF, es, gk, ell, RV, pc, Ew, bE, afd, gZ, gy, Lb, auk, bC, MC, i, X, jr, auy); #c1(KRAS) c2(NP_203524) c3(GTPase KRas isoform a) c4(228402) c5(228403, 228404, 228405, 228406, 228407, 228408) c6( ) c7(atT, T, aC, aEn, bkx, pf, yS, anl, if, al, fd, bB, abD, aub, ci, bWV, aJM, ip, bv, x, aE, il, pX, aeO, Qk, auB, iv, auf, K, hu, MO, eL, cp, YL, LQ, xQ, iE, aW, bWY, Ls, hl, QB, nY, Qq, Qv, Qm, kt, NR, g, hJ, as, ahv, cm, At, zV, mK, Fu, qJ, bw, eM, nB, AM, iT, vA, PV, k, m, YK, iu, wu, sw, zX, ht, NT, n, awr, kv, bWU, fr, qN, bao, br, atW, Bl, jz, hw, Tf, gd, h, auI, yT, hn, dL, auS, bWX, hB, bXd, aEL, Yl, bXa, auM, PX, aJL, blP, ge, fe, aq, agF, kp, po, YF, zZ, cM, Yr, Zj, op, ar, iO, Qy, bP, dB, Qu, Vy, ayS, Tk, hL, bcd, gw, at, nl, qF, bkE, bD, Te, Qz, it, B, zl, aJJ, dA, eR, jp, WT, jx, aA, bWZ, hy, v, bWW, hz, yO, AO, pd, auG, q, er, eA, eJ, av, hW, nX, BJ, qK, aT, bXb, Tj, atZ, ae, ne, s, vT, NE, OV, Y, ev, Or, BA, iU, r, aMA, aJl, lo, en, bu, cl, LV, t, apx, ik, OC, ck, ov, nM, kP, Dy, auz, AH, gX, WU, anD, bsT, aws, zu, bt, vi, gV, hl, Mb, id, iW, aEh, atS, bXc, jh, auq, Fb, fu, ad, afb, bk, qC, Jm, dP, hd, F, ib, Rq, Db, yt, vJ, NS, Yo, io, Ti, vc, hP, wm, YA, fb, pq, Yj, auY, BN, BO, d, hG, qt, bXe, ff, eZ, Kf, aF, iV, bz, Hp, bF, es, gk, eU, RV, pc, Ew, bE, afd, gZ, gy, Lb, auk, bC, MC, i, X, jr, auy); #c1(KRBOX4) c2(NP_001123370) c3(KRAB domain-containing protein 4 isoform 1) c4(47068, 47069) c5(47070, 47071, 47072, 47073, 47074, 47075, 47076, 47077, 47078, 47079) c6( ) c7(jj); #c1(KRBOX4) c2(NP_001123371) c3(KRAB domain-containing protein 4 isoform 3) c4(47080) c5(47081, 47082, 47083, 47084, 47085, 47086, 47087, 47088, 47089, 47090, 47091, 47092) c6( ) c7(jj); #c1(KRBOX4) c2(NP_060246) c3(KRAB domain-containing protein 4 isoform 2) c4(194991) c5(194992, 194993, 194994, 194995, 194996) c6( ) c7(jj); #c1(KREMEN1) c2(NP_001034659) c3(kremen protein 1 isoform 3 precursor) c4(32004) c5(32005, 32006, 32007, 32008, 32009, 32010) c6( ) c7(iO, fd); #c1(KREMEN1) c2(NP_114434) c3(kremen protein 1 isoform 2 precursor) c4(222767) c5(222768, 222769, 222770, 222771, 222772, 222773) c6( ) c7(iO, fd); #c1(KREMEN1) c2(XP_011528731) c3(kremen protein 1 isoform X1) c4(324038, 324039) c5(324040, 324041, 324042, 324043, 324044, 324045, 324046, 324047, 324048, 324049, 324050, 324051) c6( ) c7(iO, fd); #c1(KRIT1) c2(NP_001013424) c3(krev interaction trapped protein 1 isoform 2) c4(25539, 25540, 25541, 25542) c5(25543, 25544, 25545, 25546, 25547, 25548, 25549, 25550, 25551, 25552, 25553, 25554, 25555, 25556, 25557, 25558, 25559, 25560, 25561, 25562, 25563, 25564, 25565, 25566, 25567, 25568, 25569, 25570, 25571, 25572, 25573, 25574, 25575, 25576, 25577, 25578, 25579, 25580, 25581, 25582, 25583, 25584, 25585, 25586, 25587, 25588, 25589, 25590, 25591, 25592, 25593, 25594, 25595, 25596, 25597, 25598, 25599) c6( ) c7(bM, T, qb, bXg, fd, jh, bXh, bS, v, ct, bxj, ci, pK, fr, Cb, rc, ad, bXi, eA, Jm, ip, Mo, ft, ib, Yc, Ot, lC, aT, io, h, dL, K, hB, ae, eL, cp, d, s, il, aW, Y, ff, CO, en, Z, t, cm, op, es, bXk, ac, iA, bXf, vA, m, azr, xh, gT, B); #c1(KRIT1) c2(XP_011514963) c3(krev interaction trapped protein 1 isoform X1) c4(307716, 307717, 307718, 307719, 307720, 307721, 307722, 307723, 307724, 307725, 307726) c5(307727, 307728, 307729, 307730, 307731, 307732, 307733, 307734, 307735, 307736, 307737, 307738, 307739, 307740, 307741, 307742, 307743, 307744, 307745, 307746, 307747, 307748, 307749, 307750, 307751, 307752, 307753, 307754, 307755, 307756, 307757, 307758, 307759, 307760, 307761, 307762, 307763, 307764, 307765, 307766, 307767, 307768, 307769, 307770, 307771, 307772) c6( ) c7(bM, T, qb, bXg, fd, jh, bXh, bS, v, ct, bXj, ci, pK, fr, Cb, rc, ad, bXi, eA, Jm, ip, Mo, ft, ib, Yc, Ot, lC, aT, io, h, dL, K, hB, ae, eL, cp, d, s, ill, aW, Y, ff, CQ, en, Z, t, cm, op, es, bXk, ac, iA, bXf, vA, m, azr, xh, gT, B); #c1(KRT1) c2(NP_006112) c3(keratin, type II cytoskeletal 1) c4(162123) c5(162124, 162125, 162126, 162127, 162128, 162129) c6( ) c7(bnC, bXl, lg, bik, hk, bAH, bXo, ff, bXp, by, bXm, baP, bvc, K, lQ, ci, en, bXn, bXq, dr, arO); #c1(KRT10) c2(NP_000412) c3(keratin, type I cytoskeletal 10) c4(8661) c5(8662, 8663, 8664, 8665, 8666, 8667) c6( ) c7(bXr, bnC, hk, ff, ht, Pg, ew, bXs, bXm, by, rO, baP, K, IQ, hu, en, Pk, bkE, B, bXt, arO); #c1(KRT12) c2(NP_000214) c3(keratin, type I cytoskeletal (2) c4(4642) c5(4643, 4644, 4645, 4646, 4647, 4648) c6( ) c7(aOu, PK, bXv, la, zN, bXu); #c1(KRT13) c2(NP_002265) c3(keratin, type I cytoskeletal 13 isoform b) c4(127097, 127098) c5(127099, 127100, 127101, 127102, 127103, 127104, 127105, 127106, 127107, 127108, 127109, 127110, 127111, 127112) c6( ) c7(aEj, pf, aW, ff, aT, bXw, en, g, it); #c1(KRT13) c2(NP_705694) c3(keratin, type I cytoskeletal 13 isoform a) c4(246268) c5(246269, 246270, 246271, 246272, 246273, 246274) c6( ) c7(aEj, pf, aW, ff, al, bXw, en, g, it); #c1(KRT14) c2(NP_000517) c3(keratin, type I cytoskeletal 14) c4(10897) c5(10898, 10899, 10900, 10901, 10902, 10903, 10904, 10905) c6(10906, 10907) c7(T, arM, biF, if, ht, jx, it, ci, bXA, qN, XM, ad, hX, bnC, Bl, lg, aT, K, lQ, asT, arO, bPf, bTH, aW, ff, cr, bXB, mo, bXx, bXy, en, g, dr, Ta, kP, eM, bXz, YH, bXC, cn, Tc); #c1(KRT15) c2(NP_002266) c3(keratin, type I cytoskeletal 15) c4(127113, 127114) c5( ) c6( ) c7(T, bPf, lg, aW, tT, hk, afQ, qK, aT, dL, oH, ci, Dz, bAf, Pr, bXD, acG); #c1(KRT15) c2(XP_005257402) c3(keratin, type I cytoskeletal 15 isoform X1) c4(275075, 275076) c5(275077, 275078, 275079, 275080, 275081, 275082, 275083, 275084, 275085, 275086, 275087, 275088, 275089, 275090, 275091, 275092, 275093, 275094, 275095, 275096, 275097, 275098, 275099, 275100, 275101, 275102, 275103, 275104) c6( ) c7(T, bPf, lg, aW, tT, hk, afd, qK, aT, dL, oH, ci, Dz, bAf, Pr, bXD, acG); #c1(KRT16) c2(NP_005548) c3(keratin, type I cytoskeletal 16) c4(157355) c5(157356, 157357, 157358, 157359, 157360, 157361) c6( ) c7(bL, T, bXE, bis, aw, ht, cr, bXF, baP, aj, dL, v, hu, ei, bCv, adg, y, bvc, asS, J, akE); #c1(KRT17) c2(NP_000413) c3(keratin, type I cytoskeletal 17) c4(8668, 8669, 8670, 8671) c5(8672, 8673, 8674, 8675, 8676, 8677, 8678, 8679, 8680, 8681, 8682, 8683, 8684, 8685, 8686, 8687, 8688, 8689, 8690, 8691, 8692, 8693, 8694, 8695) c6( ) c7(Dw, T, by, ZA, jE, aml, ad, hX, aE, Db, afQ, aT, cU, ae, dx, arO, qw, aW, ff, cr, en, g, t, ik, bz, eM, wE, Nl, bvc, m, adg, gT, it); #c1(KRT18) c2(NP_000215) c3(keratin, type I cytoskeletal 18) c4(4649) c5(4650, 4651, 4652, 4653, 4654, 4655, 4656, 4657, 4658, 4659) c6( ) c7(T, gU, al, aA, v, ci, bh, pz, aY, XM, AO, fU, b, F, ft, aE, jz, aT, rO, Tj, ae, bc, eL, bXG, aW, ff, fc, hl, fQ, aq, fe, en, g, t, KM, eM, bE, wR, m, cP, i, hE, B, vi); #c1(KRT19) c2(NP_002267) c3(keratin, type I cytoskeletal 19) c4(127115, 127116) c5(127117, 127118, 127119, 127120, 127121, 127122, 127123, 127124, 127125, 127126, 127127, 127128) c6( ) c7(bM, T, id, jz, aSP, bXJ, yk, if, al, jh, aA, sM, n, ajp, oY, fr, ci, qN, pd, q, ad, hX, av, mj, hd, fX, aE, iu, Db, yt, lv, nv, pX, afQ, py, gd, aT, K, Dz, ae, bc, ave, li, lU, bXH, s, aW, ff, r, ahJ, aq, nY, kp, aF, en, g, cm, cM, bz, Qx, iK, ck, qJ, gk, iO, nM, bXl, eM, nB, hk, wE, it, B, KG, vi); #c1(KRT2) c2(NP_000414) c3(keratin, type II cytoskeletal 2 epidermal) c4(8696) c5(8697, 8698, 8699, 8700, 8701, 8702) c6( ) c7(bnC, bAH, ra); #c1(KRT20) c2(NP_061883) c3(keratin, type I cytoskeletal 20) c4(200932) c5(200933, 200934, 200935, 200936, 200937, 200938, 200939, 200940) c6( ) c7(bM, T, aC, vf, if, fd, bB, ci, NF, x, aE, auB, DY, pk, eL, aW, axu, LZ, g, mK, iT, nB, aep, cj, k, gn, fy, aov, wu, yG, zj, n, aj, qN, aHj, ai, ft, jz, hw, bXL, gd, h, eV, dL, ey, bxg, OV, mR, A, bAF, bub, Og, bXK, yq, xe, alj, Wt, dB, ajd, HL, FL, qc, fM, dA, jx, eX, aA, V, q, J, eJ, zn, XX, lg, nv, LY, aT, rE, af, aol, sb, s, NE, iU, xR, Y, lC, r, lH, en, cl, pb, OC, lY, ck, rR, iM, kP, Zg, nG, gX, bt, Ze, ao, id, Ke, ob, aD, iW, aEh, bXN, jh, bS, mC, bkO, dP, hd, fx, CJ, qh, oW, wm, amY, fb, aHp, gj, je, ff, Of, eZ, aF, iV, LB, bF, bXM, bE, y, X); #c1(KRT23) c2(NP_056330) c3(keratin, type I cytoskeletal 23 isoform 1) c4(187128) c5(187129, 187130, 187131, 187132, 187133, 187134, 187135, 187136) c6( ) c7(aE, bE, r, bB, uz, at, dL, cP, g, q); #c1(KRT23) c2(XP_005257257) c3(keratin, type I cytoskeletal 23 isoform X1) c4(275008) c5(275009, 275010, 275011, 275012, 275013) c6( ) c7(aE, bE, r, bB, uz, at, dL, cP, g, q); #c1(KRT3) c2(NP_476429) c3(keratin, type II cytoskeletal 3) c4(230562, 230563) c5(230564, 230565, 230566, 230567, 230568, 230569, 230570, 230571, 230572, 230573, 230574, 230575) c6( ) c7(ab, kl, aOu, bXv, bXO); #c1(KRT31) c2(NP_002268) c3(keratin, type I cuticular Ha1) c4(127129) c5(127130, 127131, 127132, 127133, 127134, 127135) c6( ) c7(bL, T, ZH, CN, iT, ds, bXP, h, aNp, Pd, bt, jr); #c1(KRT32) c2(NP_002269) c3(keratin, type I cuticular Ha2) c4(127136) c5(127137, 127138, 127139, 127140, 127141, 127142) c6( ) c7(fr); #c1(KRT34) c2(NP_066293) c3(keratin, type I cuticular Ha4) c4(207114) c5(207115, 207116, 207117, 207118, 207119, 207120, 207121) c6( ) c7(ci); #c1(KRT34) c2(XP_011523095) c3(keratin, type I cuticular Ha4 isoform X1) c4(317604) c5(317605, 317606, 317607, 317608, 317609) c6( ) c7(ci); #c1(KRT35) c2(NP_002271) c3(keratin, type I cuticular Ha5) c4(127143, 127144) c5(127145, 127146, 127147, 127148, 127149, 127150, 127151, 127152, 127153, 127154, 127155, 127156) c6( ) c7(vi); #c1(KRT4) c2(NP_002263) c3(keratin, type II cytoskeletal 4) c4(127053, 127054, (27055) c5(127056, 127057, 127058, 127059, 127060, 127061, 127062, 127063, 127064, 127065, 127066, 127067, 127068, 127069, 127070, 127071, 127072) c6( ) c7(T, it, bXw); #c1(KRT5) c2(NP_000415) c3(keratin, type II cytoskeletal 5) c4(8703, 8704, 8705) c5(8706, 8707, 8708, 8709, 8710, 8711, 8712, 8713, 8714, 8715, 8716, 8717, 8718, 8719, 8720, 8721, 8722, 8723) c6( ) c7(T, arM, bXl, biF, e, aw, by, j, ci, zP, XM, bXq, ib, aew, lg, afQ, qK, aT, iv, dL, lQ, Tj, zy, eL, eu, zQ, bTH, aW, Y, oV, aEl, iw, cr, ff, aq, ark, bXB, bXQ, bXx, pl, en, bz, g, dr, iK, sS, ac, eM, hk, DW, rF, in, arO, B, Dc); #c1(KRT6A) c2(NP_005545) c3 (keratin, type II cytoskeletal 6A) c4(157306, 157307) c5(157308, 157309, 157310, 157311, 157312, 157313, 157314, 157315, 157316, 157317, 157318, 157319) c6( ) c7(aW, bXF, bXR, aT, aA, dL, aF, lQ, bvc, bCv, gT, aue); #c1(KRT6B) c2(NP_005546) c3(keratin, type II cytoskeletal 6B) c4(157320, 157321) c5(157322, 157323, 157324, 157325, 157326, 157327, 157328, 157329, 157330, 157331, 157332, 157333) c6( ) c7(eM, bXS, ff, bvc, ci, en); #c1(KRT6C) c2(NP_775109) c3(keratin, type II cytoskeletal 6C) c4(249585, 249586) c5(249587, 249588, 249589, 249590, 249591, 249592, 249593, 249594, 249595, 249596, 249597, 249598) c6( ) c7(bvc, bXT, bCv); #c1(KRT7) c2(NP_005547) c3(keratin, type II cytoskeletal 7) c4(157334, 157335, 157336) c5(157337, 157338, 157339, 157340, 157341, 157342, 157343, 157344, 157345, 157346, 157347, 157348, 157349, 157350, 157351, 157352, 157353, 157354) c6( ) c7(bdo, T, fM, dA, Uc, pf, iW, ht, oA, KY, bXU, bcE, bS, gf, ct, ci, v, fr, zP, hF, rP, aQF, wD, x, PW, RU, hw, yt, lv, Tf, qh, aql, kH, aT, hn, dL, K, zg, hu, Tj, bXV, eL, bc, il, zl, Yj, je, s, aW, Y, ff, apK, kp, en, Eg, Z, t, g, bz, pb, Qx, iV, hq, ck, rR, iM, iT, pg, dW, zv, k, adg, y, bt, B); #c1(KRT71) c2(NP_258259) c3(keratin, type II cytoskeletal 71) c4(228683) c5(228684, 228685, 228686, 228687, 228688, 228689) c6( ) c7(zP, alu, zQ, ci, afB); #c1(KRT72) c2(NP_001139698) c3(keratin, type II cytoskeletal 72 isoform 2) c4(59753) c5(59754, 59755, 59756, 59757, 59758, 59759) c6( ) c7(adg); #c1(KRT72) c2(NP_542785) c3(keratin, type II cytoskeletal 72 isoform 1) c4(233082) c5(233083, 233084, 233085, 233086, 233087, 233088, 233089, 233090, 233091, 233092) c6( ) c7(adg); #c1(KRT72) c2(XP_011536245) c3(keratin, type II cytoskeletal 72 isoform X3) c4( ) c5(333587) c6( ) c7(adg); #c1(KRT72) c2(XP_011536246) c3(keratin, type II cytoskeletal 72 isoform X4) c4(333588) c5(333589, 333590, 333591, 333592, 333593) c6( ) c7(adg); #c1(KRT74) c2(XP_011536204) c3(keratin, type II cytoskeletal 74 isoform X1) c4(333498) c5(333499, 333500, 333501, 333502, 333503, 333504) c6( ) c7(bXW, bXY, ac, alu, bXX, e, aw, afB, j, bKO, cd, eu); #c1(KRT75) c2(NP_004684) c3(keratin, type II cytoskeletal 75) c4(148822) c5(148823, 148824, 148825, 148826, 148827, 148828) c6( ) c7(bEY, afB, bXZ); #c1(KRT76) c2(NP_056932) c3(keratin, type II cytoskeletal 2 oral) c4(188143) c5(188144, 188145, 188146, 188147, 188148, 188149, 188150) c6( ) c7(T, Ea, aHZ); #c1(KRT78) c2(NP_001287743) c3(keratin, type II cytoskeletal 78 isoform 2) c4(112760, 112761) c5(112762, 112763, 112764, 112765, 112766, 112767, 112768, 112769, 112770, 112771, 112772, 112773, 112774, 112775, 112776, 112777) c6( ) c7(ad); #c1(KRT78) c2(NP_775487) c3(keratin, type II cytoskeletal 78 isoform 1) c4(250062) c5(250063, 250064, 250065, 250066, 250067, 250068, 250069) c6( ) c7(ad); #c1(KRT8) c2(NP_001243211) c3(keratin, type II cytoskeletal 8 isoform 1) c4(90175, 90176) c5(90177, 90178, 90179, 90180, 90181, 90182, 90183, 90184, 90185, 90186, 90187, 90188, 90189, 90190, 90191, 90192, 90193) c6( ) c7(T, aC, id, fG, Or, al, aA, bB, fr, ci, bh, asF, iG, AO, ad, hX, av, bkq, b, F, ft, aE, aT, ji, K, aSX, ae, bYa, af, bc, eL, bXG, aW, ff, hl, fQ, aq, fe, aF, en, g, iV, f, cm, bz, KM, LB, ar, gk, bP, eM, bE, hk, zl, aS, i, ra, B, ao); #c1(KRT8) c2(NP_002264) c3(keratin, type II cytoskeletal 8 isoform 2) c4(127073, 127074, 127075) c5(127076, 127077, 127078, 127079, 127080, 127081, 127082, 127083, 127084, 127085, 127086, 127087, 127088, 127089, 127090, 127091, 127092, 127093, 127094, 127095, 127096) c6( ) c7(T, aC, id, fG, Or, al, aA, bB, fr, ci, bh, asf, iG, AO, ad, hX, av, bkq, b, F, ft, aE, aT, ji, K, aSX, ae, bYa, af, bc, eL, bXG, aW, ff, hl, fQ, aq, fe, af, en, g, iV, f, cm, bz, KM, LB, ar, gk, bP, eM, bE, hk, zl, aS, i, ra, B, ao); #c1(KRT80) c2(NP_001074961) c3(keratin, type II cytoskeletal 80 isoform K80.1) c4(37153) c5(37154, 37155, 37156, 37157, 37158, 37159, 37160) c6( ) c7(bnC, bis, afB, by, bvc, ra, arO); #c1(KRT80) c2(NP_872313) c3(keratin, type II cytoskeletal 80 isoform K80) c4(256101) c5(256102, 256103, 256104, 256105, 256106, 256107) c6( ) c7(bnC, bis, afB, by, bvc, ra, arO); #c1(KRT81) c2(XP_011536636) c3(keratin, type II cuticular Hb1 isoform X1) c4(334115, 334116) c5(334117, 334118, 334119, 334120, 334121, 334122, 334123, 334124, 334125, 334126, 334127, 334128) c6( ) c7(eM, bis, Qu, ff, n, ci, en, g); #c1(KRT82) c2(NP_149022) c3(keratin, type II cuticular Hb2) c4(226784) c5(226785, 226786, 226787, 226788, 226789, 226790) c6( ) c7(ac); #c1(KRT83) c2(NP_002273) c3(keratin, type II cuticular Hb3) c4(127157) c5(127158, 127159, 127160, 127161, 127162, 127163) c6( ) c7(ac, bis); #c1(KRT85) c2(NP_001287739) c3(keratin, type Il cuticular Hb5 isoform 2) c4(112746) c5(112747, 112748, 112749, 112750, 112751, 112752) c6( ) c7(bKO, ac); #c1(KRT85) c2(NP_002274) c3(keratin, type II cuticular Hb5 isoform 1) c4(127164) c5(127165, 127166, 127167, 127168, 127169, 127170) c6( ) c7(bKO, ac); #c1(KRT86) c2(XP_005268923) c3(keratin, type II cuticular Hb6 isoform X2) c4(282190, 282191, 282192, 282193) c5(282194, 282195, 282196) c6( ) c7(aOu, bis, afB, iV, bYb, dr); #c1(KRT86) c2(XP_011536640) c3(keratin, type II cuticular Hb6 isoform X1) c4( ) c5(334129, 334130, 334131, 334132, 334133, 334134, 334135, 334136, 334137, 334138, 334139, 334140, 334141, 334142) c6( ) c7(aOu, bis, afB, iV, bYb, dr); #c1(KRT9) c2(NP_000217) c3(keratin, type I cytoskeletal 9) c4(4660) c5(4661, 4662, 4663, 4664, 4665, 4666) c6( ) c7(bnC, ff, buZ, mx, by, bvc, bYd, lQ, m, en, t, bYc, arO); #c1(KRTAP11-1) c2(NP_787054) c3(keratin-associated protein (1-1) c4(252026) c5(252027, 252028, 252029, 252030, 252031, 252032) c6( ) c7(dY); #c1(KRTAP5-1) c2(NP_001005922) c3(keratin-associated protein 5-1) c4(22178) c5(22179) c6( ) c7(bnC); #c1(KRTAP5-9) c2(NP_005544) c3(keratin-associated protein 5-9) c4(157301) c5(157302, 157303, 157304, 157305) c6( ) c7(y, ai); #c1(KSR1) c2(XP_006722214) c3(kinase suppressor of Ras 1 isoform X2) c4(294635, 294636) c5(294637, 294638, 294639, 294640, 294641, 294642, 294643, 294644, 294645, 294646, 294647, 294648) c6( ) c7(ck, T, aC, aE, gT, aW, aT, ci, m, t, aly); #c1(KSR1) c2(XP_011523731) c3(kinase suppressor of Ras 1 isoform X3) c4(318185, 318186) c5(318187, 318188, 318189, 318190, 318191, 318192, 318193, 318194, 318195, 318196, 318197, 318198) c6( ) c7(ck, T, aC, aE, gT, aW, aT, ci, m, t, aQy); #c1(KSR1) c2(XP_011523732) c3(kinase suppressor of Ras I isoform X5) c4(318199) c5(318200, 318201, 318202, 318203, 318204, 318205) c6( ) c7(ck, T, aC, aE, gT, aW, al, ci, m, t, aQy); #c1(KSR1) c2(XP_011523735) c3(kinase suppressor of Ras 1 isoform X9) c4(318206) c5(318207, 318208, 318209, 318210, 318211) c6( ) c7(ck, T, aC, aE, gT, aW, aT, ci, m, t, aly); #c1(KSR2) c2(NP_775869) c3(kinase suppressor of Ras 2) c4(250517, 250518) c5(250519, 250520, 250521, 250522, 250523) c6( ) c7(bL, hH, bt, v); #c1(KSR2) c2(XP_011536526) c3(kinase suppressor of Ras 2 isoform X1) c4( ) c5(333892, 333893, 333894, 333895, 333896, 333897, 333898) c6( ) c7(bL, hH, bt, v); #c1(KSR2) c2(XP_011536528) c3(kinase suppressor of Ras 2 isoform X3) c4(333899) c5(333900, 333901, 333902, 333903, 333904, 333905) c6( ) c7(bL, hH, bt, v); #c1(KSR2) c2(XP_011536530) c3(kinase suppressor of Ras 2 isoform X5) c4(333906) c5(333907, 333908, 333909, 333910, 333911, 333912) c6( ) c7(bL, hH, bt, v); #c1(KSR2) c2(XP_011536532) c3(kinase suppressor of Ras 2 isoform X7) c4( ) c5(333913) c6( ) c7(bL, hH, bt, v); #c1(KTN1) c2(NP_001072989) c3(kinectin isoform a) c4(35481, 35482) c5(35483, 35484, 35485, 35486, 35487, 35488, 35489, 35490, 35491, 35492, 35493, 35494, 35495, 35496, 35497, 35498, 35499, 35500) c6( ) c7(dW, km); #c1(KTN1) c2(NP_001072990) c3(kinectin isoform b) c4(35501) c5(35502, 35503, 35504, 35505, 35506, 35507, 35508, 35509, 35510, 35511, 35512, 35513, 35514, 35515) c6( ) c7(dW, km); #c1(KTN1) c2(NP_001257943) c3(kinectin isoform d) c4(95842) c5( ) c6( ) c7(dW, km); #c1(KTN1) c2(NP_004977) c3(kinectin isoform c) c4(151563) c5(151564, 151565, 151566, 151567, 151568, 151569) c6( ) c7(dW, km); #c1(KTN1) c2(XP_006720204) c3(kinectin isoform X6) c4( ) c5(293310, 293311, 293312, 293313, 293314, 293315, 293316, 293317, 293318, 293319, 293320, 293321) c6( ) c7(dW, km); #c1(KYNU) c2(NP_001028170) c3(kynureninase isoform b) c4(30323) c5(30324, 30325, 30326, 30327, 30328, 30329) c6( ) c7(bYe, ey, qa, U, cz); #c1(KYNU) c2(NP_001186170) c3(kynureninase isoform a) c4(77224) c5(77225, 77226, 77227, 77228, 77229, 77230, 77231, 77232, 77233, 77234, 77235, 77236, 77237, 77238, 77239, 77240, 77241, 77242, 77243, 77244, 77245, 77246, 77247, 77248, 77249, 77250, 77251, 77252, 77253, 77254, 77255, 77256, 77257, 77258, 77259, 77260, 77261, 77262, 77263, 77264, 77265, 77266, 77267, 77268, 77269, 77270, 77271, 77272, 77273, 77274, 77275, 77276, 77277, 77278, 77279, 77280, 77281, 77282, 77283, 77284, 77285, 77286, 77287, 77288, 77289, 77290, 77291, 77292, 77293, 77294, 77295, 77296, 77297, 77298, 77299, 77300, 77301, 77302, 77303, 77304, 77305, 77306, 77307, 77308, 77309, 77310, 77311, 77312, 77313, 77314, 77315, 77316, 77317, 77318, 77319, 77320) c6( ) c7(bYe, ey, qa, U, cz); #c1(KYNU) c2(XP_011510407) c3 (kynureninase isoform X2) c4( ) c5(300977) c6( ) c7(bYe, ey, qa, U, cz); #c1(L1CAM) c2(NP_000416) c3(neural cell adhesion molecule L1 isoform 1 precursor) c4(8724, 8725, 8726, 8727) c5(8728, 8729, 8730, 8731, 8732, 8733, 8734, 8735, 8736, 8737, 8738, 8739, 8740, 8741, 8742, 8743, 8744, 8745, 8746, 8747, 8748, 8749, 8750, 8751, 8752, 8753, 8754, 8755, 8756) c6( ) c7(bYi, T, ul, AB, Mb, aio, Ay, aA, n, bB, bmf, ct, ci, nA, fr, bYh, MT, q, aR, eA, eJ, dP, ft, no, hd, aV, aE, Eo, aT, adB, bYj, fb, yj, ahE, bYf, af, d, bYg, aW, Y, ff, r, nf, fe, kp, aF, ahl, en, bu, cl, t, g, bz, cm, bJ, hq, ov, es, eM, nB, dB, bE, iA, m, bee, X); #c1(L1CAM) c2(NP_001137435) c3(neural cell adhesion molecule L1 isoform 3 precursor) c4(55697) c5(55698, 55699, 55700, 55701, 55702, 55703, 55704, 55705, 55706, 55707, 55708, 55709, 55710, 55711) c6( ) c7(bYi, T, ul, AB, Mb, aio, Ay, aA, n, bB, bmf, ct, ci, nA, fr, bYh, MT, q, aR, eA, eJ, dP, ft, no, hd, aV, aE, Eo, aT, adB, bYj, fb, yj, ahE, bYf, af, d, bYg, aW, Y, ff, r, nf, fe, kp, aF, ahl, en, bu, cl, t, g, bz, cm, bJ, hq, ov, es, eM, nB, dB, bE, iA, m, bee, X); #c1(L1CAM) c2(NP_076493) c3(neural cell adhesion molecule L1 isoform 2 precursor) c4(214164, 214165, 214166) c5(214167, 214168, 214169, 214170, 214171, 214172, 214173, 214174, 214175, 214176, 214177, 214178, 214179, 214180, 214181, 214182, 214183, 214184, 214185) c6( ) c7(bYi, T, ul, AB, Mb, aio, Ay, aA, n, bB, bmf, ct, ci, nA, fr, bYh, MT, q, aR, eA, eJ, dP, ft, no, hd, aV, aE, Eo, aT, adB, bYj, fb, yj, ahE, bYf, af, d, bYg, aW, Y, ff, r, nf, fe, kp, aF, ahl, en, bu, cl, t, g, bz, cm, bJ, hq, ov, es, eM, nB, dB, bE, iA, m, bee, X); #c1(L2HGDH) c2(XP_005268132) c3(L-2-hydroxyglutarate dehydrogenase, mitochondrial isoform X1) c4(281612, 281613) c5(281614, 281615, 281616, 281617, 281618, 281619, 281620, 281621, 281622, 281623, 281624, 281625, 281626, 281627, 281628, 281629) c6( ) c7(d, dB, ha, nA, bcV, bYk); #c1(L3MBTL1) c2(NP_056293) c3(lethal(3)malignant brain tumor-like protein 1 isoform I) c4(186958, 186959, 186960, 186961, 186962) c5(186963, 186964, 186965, 186966, 186967, 186968, 186969, 186970, 186971, 186972, 186973, 186974, 186975, 186976, 186977, 186978, 186979, 186980, 186981, 186982, 186983, 186984, 186985, 186986, 186987, 186988, 186989, 186990, 186991, 186992, 186993, 186994, 186995, 186996, 186997) c6( ) c7(ck, T, iT, dB, dz, pm, hf, bw, bt); #c1(L3MBTL1) c2(NP_115479) c3(lethal(3)malignant brain tumor-like protein 1 isoform II) c4(222841, 222842, 222843, 222844) c5(222845, 222846, 222847, 222848, 222849, 222850, 222851, 222852, 222853, 222854, 222855, 222856, 222857, 222858, 222859, 222860, 222861, 222862, 222863, 222864, 222865, 222866, 222867) c6( ) c7(ck, T, iT, dB, dz, pm, hf, bw, bt); #c1(L3MBTL2) c2(NP_113676) c3(lethal(3)malignant brain tumor-like protein 2) c4(222056) c5(222057, 222058, 222059, 222060, 222061, 222062) c6( ) c7(dB, bt, jf, pm); #c1(L3MBTL2) c2(XP_011528722) c3(lethal(3)malignant brain tumor-like protein 2 isoform X1) c4(324010) c5(324011, 324012, 324013, 324014, 324015, 324016) c6( ) c7(dB, bt, jf, pm); #c1(L3MBTL3) c2(NP_001007103) c3 (lethal(3)malignant brain tumor-like protein 3 isoform b) c4(22491) c5(22492, 22493, 22494, 22495, 22496, 22497, 22498, 22499, 22500, 22501, 22502, 22503, 22504, 22505) c6( ) c7(bL, jf); #c1(L3MBTL3) c2(NP_115814) c3(lethal(3)malignant brain tumor-like protein 3 isoform a) c4(224049, 224050) c5(224051, 224052, 224053, 224054, 224055, 224056, 224057, 224058, 224059, 224060, 224061, 224062, 224063, 224064, 224065, 224066, 224067, 224068, 224069, 224070, 224071) c6( ) c7(bL, jf); #c1(L3MBTL4) c2(NP_775735) c3(lethal(3)malignant brain tumor-like protein 4) c4(250107) c5(250108, 250109, 250110, 250111, 250112, 250113, 250114, 250115, 250116, 250117) c6( ) c7(aC, ac); #c1(L3MBTL4) c2(XP_005258223) c3(lethal(3)malignant brain tumor-like protein 4 isoform X4) c4(275586) c5(275587, 275588, 275589, 275590, 275591, 275592) c6( ) c7(aC, ac); #c1(LACC1) c2(XP_006719829) c3(laccase domain-containing protein 1 isoform X1) c4(293008) c5(293009, 293010, 293011, 293012, 293013, 293014, 293015, 293016, 293017, 293018) c6( ) c7(AW, acC, fe, iV, hH, acD, cd); #c1(LACC1) c2(XP_011533237) c3(laccase domain-containing protein 1 isoform X3) c4(329357) c5(329358, 329359, 329360, 329361, 329362, 329363) c6( ) c7(AW, acC, fe, iV, hH, acD, cd); #c1(LACE1) c2(NP_660358) c3 (lactation elevated protein 1) c4(241089) c5(241090, 241091, 241092, 241093, 241094, 241095) c6( ) c7(bK); #c1(LACRT) c2(NP_150593) c3(extracellular glycoprotein Iacritin precursor) c4(228117) c5(228118, 228119, 228120, 228121, 228122, 228123) c6( ) c7(bYI, aT); #c1(LACTB) c2(NP_116246) c3(serine beta-lactamase-like protein LACTB, mitochondrial isoform a precursor) c4(225941, 225942) c5(225943, 225944, 225945, 225946, 225947, 225948) c6( ) c7(v, fS); #c1(LACTB) c2(NP_741982) c3(serine beta-lactamase-like protein LACTB, mitochondrial isoform b precursor) c4( ) c5(247517, 247518, 247519, 247520, 247521, 247522) c6( ) c7(v, fS); #c1(LAD1) c2(NP_005549) c3(ladinin-1) c4(157362, 157363) c5(157364, 157365, 157366, 157367, 157368, 157369, 157370, 157371, 157372, 157373, 157374, 157375, 157376, 157377, 157378, 157379) c6(157380, 157381, 157382, 157383) c7(bL, yG, bfM, n, aj, nA, bfO, XM, J, aRG, aRM, dv, A, arl, Cw, oV, awv, arJ, dr, aRH, ck, uz, Km, XN); #c1(LAG3) c2(NP_002277) c3(lymphocyte activation gene 3 protein precursor) c4(127222) c5(127223, 127224, 127225, 127226, 127227, 127228) c6( ) c7(ck, T, A, anR, qt, jx, eX, fd, h, eV, eo, mo, AO, V, asE, au); #c1(LAIR1) c2(NP_001275955) c3(leukocyte-associated immunoglobulin-like receptor 1 isoform f) c4(107532) c5( ) c6( ) c7(ey, eZ, iT, cj); #c1(LAIR1) c2(NP_002278) c3(leukocyte-associated immunoglobulin-like receptor 1 isoform a precursor) c4(127229) c5(127230, 127231, 127232, 127233, 127234, 127235) c6( ) c7(ey, eZ, iT, cj); #c1(LAIR1) c2(NP_068352) c3(leukocyte-associated immunoglobulin-like receptor 1 isoform b precursor) c4(209318) c5(209319, 209320, 209321, 209322, 209323, 209324) c6( ) c7(ey, eZ, iT, cj); #c1(LAIR1) c2(XP_005258977) c3(leukocyte-associated immunoglobulin-like receptor 1 isoform X7) c4( ) c5(275908, 275909, 275910, 275911, 275912, 275913) c6( ) c7(ey, eZ, iT, cj); #c1(LAIR1) c2(XP_005258981) c3(leukocyte-associated immunoglobulin-like receptor 1 isoform X8) c4( ) c5(275914) c6( ) c7(ey, eZ, iT, cj); #c1(LAIR1) c2(XP_011545830) c3 (leukocyte-associated immunoglobulin-like receptor 1 isoform X2) c4( ) c5(344761) c6( ) c7(ey, eZ, iT, cj); #c1(LAIR2) c2(NP_002279) c3 (leukocyte-associated immunoglobulin-like receptor 2 isoform a precursor) c4(127236) c5(127237, 127238, 127239, 127240, 127241, 127242, 127243) c6(127244, 127245) c7(mR, bX); #c1(LAIR2) c2(NP_067154) c3(leukocyte-associated immunoglobulin-like receptor 2 isoform b precursor) c4(208971) c5(208972, 208973, 208974, 208975, 208976, 208977, 208978) c6(208979, 208980) c7(mR, bX); #c1(LALBA) c2(XP_011536644) c3(alpha-lactalbumin isoform X2) c4(334143) c5(334144) c6( ) c7(aW, boH, aHE, aT, mo); #c1(LALBA) c2(XP_011536645) c3(alpha-lactalbumin isoform X1) c4( ) c5(334145, 334146, 334147, 334148, 334149) c6( ) c7(aW, boH, aHE, aT, mo); #c1(LAMA1) c2(NP_005550) c3(laminin subunit alpha-1 precursor) c4(157384) c5(157385, 157386, 157387, 157388, 157389, 157390) c6( ) c7(bL, iu, gU, ac, lR, il, bE, nx, eZ, bB, v, nA, hF, af); #c1(LAMA1) c2(XP_011523957) c3(laminin subunit alpha-1 isoform X1) c4( ) c5(318513, 318514, 318515, 318516, 318517) c6( ) c7(bL, iu, gU, ac, lR, il, bE, nx, eZ, bB, v, nA, hF, af); #c1(LAMA1) c2(XP_011523958) c3(laminin subunit alpha-isoform X2) c4(318518, 318519) c5(318520) c6( ) c7(bL, iu, gU, ac, lR, il, bE, nx, eZ, bB, v, nA, hF, af); #c1(LAMA1) c2(XP_011523959) c3(laminin subunit alpha-1 isoform X3) c4( ) c5(318521) c6( ) c7(bL, iu, gU, ac, lR, iU, bE, nx, eZ, bB, v, nA, hF, af); #c1(LAMA2) c2(NP_000417) c3(laminin subunit alpha-2 isoform a precursor) c4(8757, 8758, 8759) c5(8760, 8761, 8762, 8763, 8764, 8765, 8766, 8767, 8768, 8769, 8770, 8771, 8772, 8773, 8774, 8775, 8776, 8777) c6( ) c7(cZ, dA, arw, fu, nA, bX, q, AW, aLs, bYm, no, Ae, fm, oj, Ak, bgr, Jn, dd, Wq, vP, VC, Y, xs, lb, r, uY, fq, c, nB, km, Be, iu, lf); #c1(LAMA2) c2(XP_011534122) c3(laminin subunit alpha-2 isoform X2) c4(330356) c5(330357, 330358, 330359, 330360, 330361, 330362) c6( ) c7(cZ, dA, arw, fu, nA, bX, q, AW, aLs, bYm, no, Ae, fm, oj, Ak, bgr, Jn, dd, Wq, vP, VC, Y, xs, lb, r, uY, fq, c, nB, km, Be, iu, lf); #c1(LAMA3) c2(NP_000218) c3(laminin subunit alpha-3 isoform 2 precursor) c4(4667) c5(4668, 4669, 4670, 4671, 4672, 4673) c6( ) c7(D, dt, T, d, s, ac, eR, byn, aw, XJ, aT, aA, aF, Zh, dy, g, bYn, aRE, arL, XN, ad); #c1(LAMA3) c2(NP_001121189) c3(laminin subunit alpha-3 isoform 3 precursor) c4(45733, 45734) c5(45735, 45736, 45737, 45738, 45739, 45740, 45741, 45742, 45743, 45744, 45745, 45746, 45747, 45748, 45749, 45750, 45751) c6( ) c7(D, dt, T, d, s, ac, eR, byn, aw, XJ, aT, aA, aF, Zh, dy, g, bYn, aRE, arL, XN, ad); #c1(LAMA3) c2(NP_001121190) c3(laminin subunit alpha-3 isoform 4 precursor) c4(45752) c5(45753, 45754, 45755, 45756, 45757, 45758) c6( ) c7(D, dt, T, d, s, ac, eR, byn, aw, XJ, aT, aA, aF, Zh, dy, g, bYn, aRE, arL, XN, ad); #c1(LAMA3) c2(NP_937762) c3(laminin subunit alpha-3 isoform 1 precursor) c4(259068) c5(259069, 259070, 259071, 259072, 259073, 259074, 259075) c6( ) c7(D, dt, T, d, s, ac, eR, byn, aw, XJ, aT, aA, aF, Zh, dy, g, bYn, aRE, arL, XN, ad); #c1(LAMA3) c2(XP_011524280) c3(laminin subunit alpha-3 isoform X1) c4(318773) c5( ) c6( ) c7(D, dt, T, d, s, ac, eR, byn, aw, XJ, aT, aA, aF, Zh, dy, g, bYn, aRE, arL, XN, ad); #c1(LAMA4) c2(NP_001098676) c3(laminin subunit alpha-4 isoform 1 precursor) c4(41413, 41414, 41415) c5(41416, 41417, 41418, 41419, 41420, 41421, 41422, 41423, 41424, 41425, 41426, 41427, 41428, 41429, 41430, 41431, 41432, 41433, 41434, 41435, 41436, 41437, 41438, 41439, 41440, 41441, 41442, 41443, 41444, 41445, 41446, 41447, 41448, 41449, 41450, 41451, 41452, 41453, 41454, 41455, 41456) c6(41457, 41458, 41459, 41460) c7(T, dt, dD, s, iU, dB, aw, mx, aT, aA, aF, bYo, ci, eq, g, eA, XN, B, ad); #c1(LAMA4) c2(NP_001098679) c3(laminin subunit alpha-4 isoform 3 precursor) c4(41461) c5(41462, 41463, 41464, 41465, 41466, 41467, 41468, 41469, 41470, 41471, 41472, 41473, 41474, 41475, 41476, 41477, 41478, 41479, 41480, 41481, 41482, 41483, 41484) c6( ) c7(T, dt, dD, s, il, dB, aw, mx, aT, aA, aF, bYo, ci, eq, g, eA, XN, B, ad); #c1(LAMA4) c2(NP_002281) c3(laminin subunit alpha-4 isoform 2 precursor) c4(127246, 127247, 127248, 127249, 127250) c5(127251, 127252, 127253, 127254, 127255, 127256, 127257, 127258, 127259, 127260, 127261, 127262, 127263, 127264, 127265, 127266, 127267, 127268, 127269, 127270) c6( ) c7(T, dt, dD, s, il, dB, aw, mx, aT, aA, aF, bYo, ci, eq, g, eA, XN, B, ad); #c1(LAMA4) c2(XP_011534123) c3(laminin subunit alpha-4 isoform X2) c4(330363, 330364, 330365, 330366) c5(330367, 330368, 330369, 330370, 330371, 330372, 330373, 330374, 330375, 330376, 330377, 330378, 330379, 330380, 330381, 330382, 330383, 330384, 330385, 330386, 330387, 330388, 330389, 330390, 330391, 330392, 330393, 330394, 330395, 330396, 330397, 330398, 330399, 330400, 330401, 330402, 330403, 330404, 330405, 330406, 330407, 330408, 330409) c6( ) c7(T, dt, dD, s, iU, dB, aw, mx, aT, aA, aF, bYo, ci, eq, g, eA, XN, B, ad); #c1(LAMA5) c2(NP_005551) c3(laminin subunit alpha-5 precursor) c4(157391) c5(157392, 157393, 157394, 157395, 157396, 157397, 157398, 157399, 157400, 157401, 157402, 157403, 157404, 157405, 157406, 157407, 157408, 157409) c6( ) c7(bM, fR, aW, r, aT, q, eA, XN, pm, aY, BB, B, ad); #c1(LAMAS) c2(XP_006723861) c3(laminin subunit alpha-5 isoform X4) c4( ) c5(295415) c6( ) c7(bM, fR, aW, r, aT, q, eA, XN, pm, aY, BB, B, ad); #c1(LAMA5) c2(XP_011527121) c3(laminin subunit alpha-5 isoform X3) c4(321626, 321627) c5( ) c6( ) c7(bM, fR, aW, r, aT, q, eA, XN, pm, aY, BB, B, ad); #c1(LAMB1) c2(NP_002282) c3(laminin subunit beta-l precursor) c4(127271, 127272, 127273) c5(127274, 127275, 127276, 127277, 127278, 127279, 127280, 127281, 127282, 127283, 127284, 127285, 127286, 127287, 127288, 127289, 127290, 127291, 127292, 127293, 127294, 127295) c6(127296) c7(dP, kn, T, dB, aW, op, FB, ha, vA, oj, aT, bYp, ci, iV, ad, bN); #c1(LAMB1) c2(XP_011514505) c3(laminin subunit beta-1 isoform X1) c4( ) c5(307210, 307211) c6( ) c7(dP, kn, T, dB, aW, op, FB, ha, vA, oj, aT, bYp, ci, iV, ad, bN); #c1(LAMB2) c2(NP_002283) c3(laminin subunit beta-2 precursor) c4(127297, 127298) c5(127299, 127300, 127301, 127302, 127303, 127304, 127305, 127306, 127307, 127308, 127309, 127310, 127311, 127312, 127313, 127314) c6( ) c7(Kl, eR, aA, ah, nA, BB, eA, ad, F, vw, wt, ayT, lR, Y, bYq, An, aF, aSO, xA, KE, vW, dB, va, dy, oQ); #c1(LAMB3) c2(NP_001121113) c3(laminin subunit beta-3 precursor) c4(45183, 45184, 45185) c5(45186, 45187, 45188, 45189, 45190, 45191, 45192, 45193, 45194, 45195, 45196, 45197, 45198, 45199, 45200, 45201, 45202, 45203, 45204, 45205, 45206, 45207, 45208, 45209, 45210, 45211, 45212) c6( ) c7(ck, li, D, T, if, ff, aA, XM, n, it, aF, XO, lQ, en, qN, aRE, arL, Ud, XN, ad); #c1(LAMB4) c2(NP_031382) c3(laminin subunit beta-4 precursor) c4(172107) c5(172108, 172109, 172110, 172111, 172112, 172113, 172114, 172115, 172116, 172117, 172118, 172119, 172120, 172121) c6(172122, 172123) c7(bN); #c1(LAMB4) c2(XP_011514277) c3(laminin subunit beta-4 isoform X2) c4(306859) c5( ) c6( ) c7(bN); #c1(LAMB4) c2(XP_011514280) c3(laminin subunit beta-4 isoform X5) c4(306860) c5(306861, 306862, 306863, 306864, 306865, 306866, 306867, 306868) c6(306869, 306870) c7(bN); #c1(LAMC1) c2(NP_002284) c3(laminin subunit gamma-1 precursor) c4(127315) c5(127316, 127317, 127318, 127319, 127320, 127321) c6( ) c7(KI, eR, RX, aA, ah, lK, BB, ad, il, le, vw, ae, af, d, lR, eZ, An, aF, aSO, zZ, t, bfP, xA, KE, vW, c, dB, dy, B); #c1(LAMC2) c2(NP_005553) c3(laminin subunit gamma-2 isoform a precursor) c4(157417, 157418) c5(157419, 157420, 157421, 157422, 157423, 157424) c6( ) c7(T, uk, Js, l, fd, ci, mF, aR, ip, lE, aXn, Fo, Qk, aU, aXg, K, rB, H, arL, eL, aXp, amN, aiV, aW, Oj, aXh, dg, g, cm, akw, bw, bb, jV, iT, aiM, cj, U, Zn, dW, m, R, act, hh, aWX, XL, aFG, OI, aFc, aXo, n, ct, aWT, eh, eW, ke, Mo, Hx, h, SH, dL, aWY, aDC, pi, alY, cC, nP, Nr, aXf, po, CQ, ajz fs, RB, bo, JM, aXv, ahC, pg, aEG, bk, eo, XN, it, B, Jo, abW, L, qc, ek, ul, dA, Nt, XJ, ti, afK, aA, cz, aWU, C, j, aRE, V, q, eA, eJ, cF, aaF, Cc, WY, hb, aql, aT, rO, an, rz, af, lr, jf, ak, oV, Y, aM, WQ, Pg, RF, aaE, en, cl, wA, t, xw, UZ, aXi, rY, dS, Cv, ck, Qi, bN, tj, aXm, c, auo, aXe, ac, agc, mG, AH, Uq, HK, ig, yW, JH, fD, ij, bt, Ze, gV, hl, qX, jh, bS, fu, aNH, XM, vN, ad, iC, sO, akJ, abg, oW, lQ, cb, rL, iM, cs, d, lR, hG, qZ, je, ff, aGS, eZ, dq, aF, xH, Fq, Z, aXs, bz, Nq, bF, D, Bv, aqM, aHL, bE, aXq, y, aXu, KS, CW, Ex); #c1(LAMC2) c2(NP_061486) c3(laminin subunit gamma-2 isoform b precursor) c4(200350) c5(200351, 200352, 200353, 200354, 200355, 200356, 200357, 200358, 200359, 200360, 200361, 200362) c6( ) c7(T, uk, Js, l, fd, ci, mF, aR, ip, lE, aXn, Fo, Qk, aU, aXg, K, rB, H, arL, eL, aXp, amN, aiV, aW, Oj, aXh, dg, g, cm, akw, bw, bb, jV, iT, aiM, cj, U, Zn, dW, m, R, act, hh, aWX, XL, aFG, Ol, aFc, aXo, n, ct, aWT, eh, eW, ke, Mo, Hx, h, SH, dL, aWY, aDC, pi, alY, cC, nP, Nr, aXf, po, CQ, ajz, fs, RB, bo, JM, aXv, ahC, pg, aEG, bk, eo, XN, it, B, Jo, abW, L, qc, ek, ul, dA, Nt, XJ, ti, afK, aA, cz, aWU, C, j, aRE, V, q, eA, eJ, cF, aaF, Cc, WY, hb, aql, aT, rO, an, rz, af, lr, jf, ak, oV, Y, aM, WQ, Pg, RF, aaE, en, cl, wA, t, xw, UZ, aXi, rY, dS, Cv, ck, Qi, bN, tj, aXm, c, auo, aXe, ac, agc, mG, AH, Uq, HK, ig, yW, JH, fD, ij, bt, Ze, gV, hl, qX, jh, bS, fu, aNH, XM, vN, ad, iC, sO, akJ, abg, oW, lQ, cb, rL, iM, cs, d, lR, hG, qZ, je, ff, aGS, eZ, dq, aF, xH, Fq, Z, aXs, bz, Nq, bF, D, Bv, aqM, aHL, bE, aXq, y, aXu, KS, CW, Ex); #c1(LAMC3) c2(NP_006050) c3(laminin subunit gamma-3 precursor) c4(161455) c5(161456, 161457, 161458, 161459, 161460, 161461, 161462, 161463, 161464, 161465, 161466, 161467) c6( ) c7(bYr, hb, cd, VX); #c1(LAMC3) c2(XP_011516423) c3(laminin subunit gamma-3 isoform X1) c4(309649) c5( ) c6( ) c7(bYr, hb, cd, VX); #c1(LAMP1) c2(NP_005552) c3(lysosome-associated membrane glycoprotein 1 precursor) c4(157410) c5(157411, 157412, 157413, 157414, 157415, 157416) c6( ) c7(T, jh, C, pz, q, eW, aJO, ds, dv, h, an, bc, ak, Y, r, as, t, ck, vl, W, eM, cj, wR, Lq, m); #c1(LAMP1) c2(XP_011535797) c3(lysosome-associated membrane glycoprotein 1 isoform X2) c4(333013) c5(333014, 333015, 333016, 333017, 333018, 333019) c6( ) c7(T, jh, C, pz, q, eW, aJO, ds, dv, h, an, bc, ak, Y, r, as, t, ck, vl, W, eM, cj, wR, Lq, m); #c1(LAMP2) c2(NP_001116078) c3(lysosome-associated membrane glycoprotein 2 isoform C precursor) c4(43383, 43384) c5(43385, 43386, 43387, 43388, 43389, 43390, 43391, 43392, 43393, 43394, 43395, 43396) c6( ) c7(T, iG, aks, nA, q, cc, adl, dv, oj, aT, aiW, vc, aWs, mL, ak, lR, aW, Bw, r, ar, bnP, mx, oh, adh, cB, hv); #c1(LAMP2) c2(NP_002285) c3(lysosome-associated membrane glycoprotein 2 isoform A precursor) c4(127322) c5(127323, 127324, 127325, 127326, 127327, 127328) c6( ) c7(T, iG, aKs, nA, q, cc, adl, dv, oj, aT, aiW, vc, aWs, ml, ak, lR, aW, Bw, r, ar, bnP, mx, oh, adh, cB, hv); #c1(LAMP2) c2(NP_054701) c3(lysosome-associated membrane glycoprotein 2 isoform B precursor) c4(177293, 177294) c5(177295, 177296, 177297, 177298, 177299, 177300, 177301, 177302, 177303, 177304, 177305, 177306) c6( ) c7(T, iG, aks, nA, q, cc, adl, dv, oj, aT, aiW, vc, aWs, ml, ak, lR, aW, Bw, r, ar, bnP, mx, oh, adh, cB, hv); #c1(LAMP3) c2(NP_055213) c3(lysosome-associated membrane glycoprotein 3 precursor) c4(179868, 179869) c5(179870, 179871, 179872, 179873, 179874, 179875) c6( ) c7(T, if, aA, n, qN, ds, aT, cU, h, bk, K, ey, an, XZ, s, aW, Y, apn, r, Ni, lo, aF, g, awi, alm, aDX, ig, awx, AH, y, iu); #c1(LAMP3) c2(XP_011510990) c3(lysosome-associated membrane glycoprotein 3 isoform X3) c4( ) c5(301452, 301453, 301454, 301455, 301456, 301457) c6( ) c7(T, if, aA, n, qN, ds, aT, cU, h, bK, K, ey, an, XZ, s, aW, Y, apn, r, Ni, lo, aF, g, awi, alm, aDX, ig, awx, AH, y, iu); #c1(LAMTOR1) c2(NP_060377) c3(ragulator complex protein LAMTOR1) c4(195695) c5(195696, 195697, 195698, 195699, 195700, 195701) c6( ) c7(ck, T, eG, eM, Y, iS, fd, bw, v, K, hF, vt, B, ip); #c1(LAMTOR2) c2(NP_001138736) c3(ragulator complex protein LAMTOR2 isoform 2) c4(57495) c5(57496, 57497, 57498, 57499, 57500, 57501) c6( ) c7(sc, T, aEj, jh, n, fr, ci, q, ad, ft, aT, dL, K, jf, aW, Y, r, aF, g, bYs, cj, EY, zA, B); #c1(LAMTOR2) c2(NP_054736) c3(ragulator complex protein LAMTOR2 isoform 1) c4(177479, 177480) c5(177481, 177482, 177483, 177484, 177485, 177486, 177487, 177488, 177489, 177490, 177491, 177492) c6( ) c7(sc, T, aEj, jh, n, fr, ci, q, ad, ft, aT, dL, K, jf, aW, Y, r, aF, g, bYs, cj, EY, zA, B); #c1(LAMTOR3) c2(NP_068805) c3(ragulator complex protein LAMTOR3 isoform 1) c4(210643) c5(210644, 210645, 210646, 210647, 210648, 210649) c6( ) c7(aW, aE, aT); #c1(LANCL1) c2(NP_001130047) c3(lanC-like protein 1) c4(52486) c5(52487, 52488, 52489, 52490, 52491, 52492, 52493, 52494, 52495, 52496, 52497, 52498, 52499, 52500, 52501, 52502, 52503, 52504, 52505, 52506, 52507, 52508) c6( ) c7(bM, A, Jr, z, Y, eE, afS, dV, r, cU, Hn, h, cr, eo, eZ, fe, afT, y, V, q); #c1(LANCL1) c2(XP_005246300) c3(lanC-like protein 1 isoform X1) c4(267029) c5(267030, 267031, 267032, 267033, 267034, 267035) c6( ) c7(bM, A, Jr, z, Y, eE, afS, dV, r, c, Hn, h, cr, eo, eZ, fe, afT, y, V, q); #c1(LANCL2) c2(NP_061167) c3(lanC-like protein 2) c4(199700) c5(199701, 199702, 199703, 199704, 199705, 199706) c6( ) c7(mj, ds, d, fe); #c1(LAP3) c2(NP_056991) c3(cytosol aminopeptidase) c4(188362) c5(188363, 188364, 188365, 188366, 188367, 188368) c6( ) c7(ck, pc, dA, cj, aT, ji, cm, bt); #c1(LAPTM4B) c2(NP_060877) c3(lysosomal-associated transmembrane protein 4B) c4(198260, 198261, 198262) c5(198263, 198264, 198265, 198266, 198267, 198268, 198269, 198270, 198271, 198272, 198273, 198274, 198275, 198276, 198277, 198278, 198279, 198280) c6( ) c7(T, if, ht, aA, bB, n, ci, qN, hX, jz, NS, aT, io, K, hu, B, aW, aq, aF, bz, bE, it, X); #c1(LARGE) c2(XP_005261889) c3(glycosyltransferase-like protein LARGE1 isoform X1) c4(277653, 277654, 277655, 277656, 277657) c5(277658, 277659, 277660, 277661, 277662, 277663, 277664, 277665, 277666, 277667, 277668, 277669, 277670, 277671, 277672, 277673, 277674, 277675, 277676, 277677, 277678, 277679, 277680, 277681, 277682, 277683) c6( ) c7(oe, xs, aw, km, aT, cd, aq, ci, bYu, bxu, xa, bYt, dR); #c1(LARGE) c2(XP_011528813) c3 (glycosyltransferase-like protein LARGE1 isoform X2) c4(324169, 324170, 324171) c5( ) c6( ) c7(De, xs, aw, km, aT, cd, aq, ci, bYu, bxu, xa, bYt, dR); #c1(LARGE) c2(XP_011528814) c3(glycosyltransferase-like protein LARGE1 isoform X3) c4(324172) c5(324173, 324174, 324175, 324176, 324177, 324178) c6( ) c7(oe, xs, aw, km, aT, cd, aq, ci, bYu, bxu, xa, bYt, dR); #c1(LARGE) c2(XP_011528816) c3(glycosyltransferase-like protein LARGE1 isoform X5) c4( ) c5(324179, 324180, 324181, 324182, 324183, 324184, 324185, 324186, 324187, 324188, 324189, 324190, 324191, 324192, 324193, 324194, 324195, 324196, 324197, 324198, 324199, 324200, 324201, 324202, 324203, 324204, 324205, 324206, 324207, 324208) c6( ) c7(oe, xs, aw, km, aT, cd, aq, ci, bYu, bxu, xa, bYt, dR); #c1(LARP1) c2(NP_056130) c3(Ia-related protein 1) c4(185815) c5(185816, 185817, 185818, 185819, 185820, 185821, 185822, 185823, 185824, 185825, 185826, 185827) c6( ) c7(T); #c1(LARP1) c2(XP_005268461) c3(Ia-related protein 1 isoform X1) c4(281835) c5(281836, 281837, 281838, 281839, 281840, 281841) c6( ) c7(T); #c1(LARP1) c2(XP_006714838) c3(Ia-related protein 1 isoform X7) c4( ) c5(288256) c6( ) c7(T); #c1(LARP1) c2(XP_011535918) c3(Ia-related protein 1 isoform X5) c4(333177) c5( ) c6( ) c7(T); #c1(LARP1) c2(XP_011535920) c3(Ia-related protein 1 isoform X8) c4(333178) c5(333179, 333180, 333181, 333182, 333183) c6( ) c7(T); #c1(LARP4) c2(NP_001164274) c3(Ia-related protein 4 isoform d) c4( ) c5(66894) c6( ) c7(kl); #c1(LARP4) c2(NP_001164275) c3(Ia-related protein 4 isoform e) c4( ) c5(66895, 66896, 66897, 66898, 66899, 66900, 66901, 66902, 66903, 66904, 66905, 66906) c6( ) c7(kl); #c1(LARP4) c2(NP_001164279) c3(Ia-related protein 4 isoform f) c4(66914) c5(66915, 66916, 66917, 66918, 66919, 66920) c6( ) c7(kl); #c1(LARP4) c2(NP_443111) c3(Ia-related protein 4 isoform a) c4(229460, 229461, 229462) c5(229463, 229464, 229465, 229466, 229467, 229468) c6( ) c7(kl); #c1(LARP4) c2(NP_954658) c3(Ia-related protein 4 isoform b) c4(261183) c5(261184, 261185, 261186, 261187, 261188, 261189) c6( ) c7(kl); #c1(LARP4) c2(NP_954660) c3(Ia-related protein 4 isoform c) c4(261190) c5(261191, 261192, 261193, 261194, 261195, 261196) c6( ) c7(kl); #c1(LARP4) c2(XP_011536132) c3(Ia-related protein 4 isoform X3) c4(333441) c5(333442, 333443, 333444, 333445, 333446, 333447) c6( ) c7(kl); #c1(LARP4) c2(XP_011536144) c3(Ia-related protein 4 isoform X10) c4(333448) c5( ) c6( ) c7(kl); #c1(LARP4) c2(XP_011536147) c3 (Ia-related protein 4 isoform X13) c4( ) c5(333449, 333450, 333451, 333452, 333453) c6( ) c7(kl); #c1(LARP6) c2(NP_060827) c3(Ia-related protein 6 isoform 1) c4(198020) c5(198021, 198022, 198023, 198024, 198025, 198026) c6( ) c7(bL, es, py, iA, aW, Y, oV, aT, dL, v, fr, eL, eA); #c1(LARP6) c2(NP_932062) c3(Ia-related protein 6 isoform 2) c4(258733, 258734) c5(258735, 258736, 258737, 258738, 258739, 258740, 258741, 258742, 258743, 258744, 258745, 258746) c6( ) c7(bL, es, py, iA, aW, Y, oV, aT, dL, v, fr, eL, eA); #c1(LARP7) c2(NP_001253968) c3(Ia-related protein 7 isoform 2) c4(94296, 94297) c5(94298, 94299, 94300, 94301, 94302, 94303, 94304, 94305, 94306, 94307, 94308, 94309, 94310, 94311, 94312, 94313, 94314, 94315) c6( ) c7(bYv, aF, avx, nA, aA); #c1(LARP7) c2(NP_057732) c3(Ia-related protein 7 isoform 1) c4(192560, 192561, 192562) c5(192563, 192564, 192565, 192566, 192567, 192568, 192569, 192570, 192571, 192572, 192573, 192574, 192575, 192576, 192577, 192578) c6( ) c7(bYv, aF, avx, nA, aA); #c1(LARS) c2(NP_064502) c3(leucine-tRNA ligase, cytoplasmic) c4(202647, 202648, 202649) c5(202650, 202651, 202652, 202653, 202654, 202655, 202656) c6( ) c7(bL, av, ac, SE, hl, bYw, Ex, aj, bk, K, px, J, ale); #c1(LARS) c2(XP_011535959) c3(leucine-tRNA ligase, cytoplasmic isoform X3) c4( ) c5(333234, 333235, 333236, 333237, 333238, 333239, 333240) c6( ) c7(bL, av, ac, SE, hl, bYw, Ex, aj, bk, K, px, J, ale); #c1(LARS2) c2(NP_056155) c3(probable leucine-tRNA ligase, mitochondrial) c4(186003, 186004, 186005) c5(186006, 186007, 186008, 186009, 186010, 186011, 186012, 186013, 186014, 186015, 186016, 186017, 186018, 186019, 186020, 186021) c6( ) c7(bL, ac, bYx, SE, Ex, aj, bK, px, J, aQe); #c1(LARS2) c2(XP_011531856) c3(probable leucine-tRNA ligase, mitochondrial isoform X2) c4( ) c5(327918, 327919, 327920, 327921, 327922, 327923) c6( ) c7(bL, ac, bYx, SE, Ex, aj, bK, px, J, ale); #c1(LAS1L) c2(NP_001164120) c3(ribosomal biogenesis protein LAS1L isoform 2) c4(66636) c5(66637, 66638, 66639, 66640, 66641, 66642) c6( ) c7(bej); #c1(LAS1L) c2(NP_001164121) c3(ribosomal biogenesis protein LAS1L isoform 3) c4(66643) c5(66644, 66645, 66646, 66647, 66648, 66649) c6( ) c7(bej); #c1(LAS1L) c2(NP_112483) c3(ribosomal biogenesis protein LAS1L isoform 1) c4(221136) c5(221137, 221138, 221139, 221140, 221141, 221142, 221143) c6( ) c7(bej); #c1(LAS1L) c2(XP_005262358) c3(ribosomal biogenesis protein LAS1L isoform X1) c4(277854) c5( ) c6( ) c7(bej); #c1(LAS1L) c2(XP_005262361) c3(ribosomal biogenesis protein LAS1L isoform X3) c4( ) c5(277855) c6( ) c7(bej); #c1(LAS1L) c2(XP_005262363) c3(ribosomal biogenesis protein LAS1L isoform X6) c4( ) c5(277856, 277857, 277858, 277859, 277860) c6( ) c7(bej); #c1(LASP1) c2(NP_001258537) c3(LIM and SH3 domain protein 1 isoform b) c4(96119) c5(96120, 96121, 96122, 96123, 96124, 96125) c6( ) c7(T, id, ci, q, ad, hX, aT, dL, K, Dz, eL, ae, jf, aW, r, eS, t, bN, kP, iT, cX, m, it, B); #c1(LASP1) c2(NP_006139) c3(LIM and SH3 domain protein 1 isoform a) c4(162329, 162330, 162331) c5(162332, 162333, 162334, 162335, 162336, 162337, 162338, 162339, 162340, 162341, 162342, 162343, 162344, 162345, 162346, 162347, 162348, 162349, 162350, 162351, 162352, 162353) c6( ) c7(T, id, ci, q, ad, hX, aT, dL, K, Dz, eL, ae, jf, aW, r, eS, t, bN, kP, iT, cX, m, it, B); #c1(LAT) c2(NP_001014987) c3(linker for activation of T-cells family member 1 isoform b) c4( ) c5(26107, 26108, 26109, 26110, 26111) c6( ) c7(bM, T, id, acU, fr, PJ, eA, ad, eW, jz, py, aaU, h, aXd, ae, beo, d, Y, ff, hl, eZ, en, ik, dr, iq, eM, z, go, iu); #c1(LAT) c2(NP_001014988) c3(linker for activation of T-cells family member 1 isoform c) c4(26112) c5(26113, 26114, 26115, 26116, 26117) c6( ) c7(bM, T, id, acU, fr, PJ, eA, ad, eW, jz, py, aaU, h, aXd, ae, beo, d, Y, ff, hl, eZ, en, ik, dr, iq, eM, z, go, iu); #c1(LAT) c2(NP_001014989) c3(linker for activation of T-cells family member 1 isoform d) c4(26118, 26119) c5(26120, 26121, 26122, 26123, 26124, 26125, 26126, 26127) c6( ) c7(bM, T, id, acU, fr, PJ, eA, ad, eW, jz, py, aaU, h, aXd, ae, beo, d, Y, ff, hl, eZ, en, ik, dr, iq, eM, z, go, iu); #c1(LAT) c2(NP_055202) c3(linker for activation of T-cells family member 1 isoform a) c4(179760, 179761) c5(179762, 179763, 179764, 179765, 179766, 179767, 179768, 179769, 179770, 179771, 179772, 179773) c6( ) c7(bM, T, id, acU, fr, PJ, eA, ad, eW, jz, py, aaU, h, aXd, ae, beo, d, Y, ff, hl, eZ, en, iK, dr, iq, eM, z, go, iu); #c1(LAT2) c2(NP_115852) c3(linker for activation of T-cells family member 2 precursor) c4(224266, 224267, 224268, 224269) c5(224270, 224271, 224272, 224273, 224274, 224275, 224276, 224277, 224278, 224279, 224280, 224281, 224282, 224283, 224284, 224285, 224286, 224287, 224288, 224289, 224290, 224291, 224292, 224293, 224294, 224295, 224296, 224297, 224298, 224299, 224300, 224301, 224302, 224303, 224304, 224305, 224306, 224307, 224308, 224309, 224310, 224311, 224312, 224313, 224314) c6( ) c7(T, py, iT, ail, ey, bt, B, hq); #c1(LATS1) c2(NP_001257448) c3(serine/threonine-protein kinase LATS1 isoform 2) c4(95421) c5(95422, 95423, 95424, 95425, 95426, 95427) c6( ) c7(T, eM, aW, aT, K, m, as, ex, t, ahv, aLl); #c1(LATS1) c2(XP_011534552) c3(serine/threonine-protein kinase LATS1 isoform X1) c4(331092) c5(331093, 331094, 331095, 331096, 331097, 331098, 331099, 331100, 331101, 331102) c6( ) c7(T, eM, aW, aT, K, m, as, ex, t, ahv, aLl); #c1(LATS2) c2(XP_005266399) c3(serine/threonine-protein kinase LATS2 isoform X1) c4(279980) c5(279981, 279982, 279983, 279984, 279985, 279986, 279987, 279988, 279989, 279990) c6( ) c7(ck, ae, T, av, eM, ht, r, cj, aT, it, K, as, hu, bz, q, ad, hS); #c1(LAX1) c2(NP_001129662) c3(lymphocyte transmembrane adapter 1 isoform b) c4(52108) c5(52109, 52110, 52111, 52112, 52113, 52114) c6( ) c7(cz); #c1(LAX1) c2(NP_060243) c3(lymphocyte transmembrane adapter 1 isoform a) c4(194953) c5(194954, 194955, 194956, 194957, 194958, 194959) c6( ) c7(cz); #c1(LAYN) c2(NP_001245319) c3(layilin isoform 1 precursor) c4(93109) c5(93110, 93111, 93112, 93113, 93114, 93115) c6( ) c7(g); #c1(LAYN) c2(NP_001245320) c3(layilin isoform 3) c4(93116) c5(93117, 93118, 93119, 93120, 93121, 93122) c6( ) c7(g); #c1(LAYN) c2(NP_849156) c3(layilin isoform 2 precursor) c4(254300, 254301, 254302) c5(254303, 254304, 254305, 254306, 254307, 254308, 254309, 254310, 254311, 254312, 254313, 254314, 254315, 254316, 254317, 254318, 254319) c6( ) c7(g); #c1(LBH) c2(NP_112177) c3(protein LBH) c4(220632) c5(220633, 220634, 220635, 220636, 220637, 220638) c6( ) c7(gZ, bM); #c1(LBP) c2(NP_004130) c3(lipopolysaccharide-binding protein precursor) c4(143819, 143820) c5(143821, 143822, 143823, 143824, 143825, 143826, 143827, 143828, 143829, 143830, 143831, 143832) c6( ) c7(gV, DM, lk, fR, aw, al, jx, fF, v, ci, C, jL, h, nm, aFi, ji, jo, ZN, dF, af, wC, B, fQ, brW, aq, afr, afd, aNn, bE, uz, Zn, cP, aDE, pK, ei, X, ao); #c1(LBR) c2(XP_005273182) c3(lamin-B receptor isoform X3) c4( ) c5(284327, 284328, 284329, 284330, 284331, 284332) c6( ) c7(bM, T, dA, fN, ew, bB, jh, EA, Jx, V, zn, btD, aE, kH, aT, h, K, bYy, ox, jj, hH, A, nO, aW, Y, bYA, CT, eZ, ks, lo, CO, g, kk, eE, bYz, aoO, Wt, ck, asE, ou, dB, bE, bjx, cj, bC, bt, iu); #c1(LBR) c2(XP_011542487) c3(lamin-B receptor isoform X1) c4(340980, 340981, 340982) c5(340983, 340984, 340985, 340986, 340987, 340988, 340989, 340990, 340991, 340992) c6( ) c7(bM, T, dA, fN, ew, bB, jh, EA, Jx, V, zn, btD, aE, kH, aT, h, K, bYy, ox, jj, hH, A, nO, aW, Y, bYA, CT, eZ, ks, lo, CO, g, kk, eE, bYz, aoO, Wt, ck, asE, ou, dB, bE, bjx, cj, bC, bt, iu); #c1(LBR) c2(XP_011542488) c3(lamin-B receptor isoform X2) c4(340993) c5(340994, 340995, 340996, 340997, 340998, 340999) c6( ) c7(bM, T, dA, fN, ew, bB, jh, EA, Jx, V, zn, btD, aE, kH, aT, h, K, bYy, ox, jj, hH, A, nO, aW, Y, bYA, CT, eZ, ks, lo, CQ, g, kk, eE, bYz, aoO, Wt, ck, asE, ou, dB, bE, bjx, cj, bC, bt, iu); #c1(LBR) c2(XP_011542489) c3(lamin-B receptor isoform X4) c4(341000) c5(341001, 341002, 341003, 341004, 341005, 341006, 341007, 341008, 341009, 341010, 341011, 341012) c6( ) c7(bM, T, dA, fN, ew, bB, jh, EA, Jx, V, zn, btD, aE, kH, aT, h, K, bYy, ox, jj, hH, A, nO, aW, Y, bYA, CT, eZ, ks, lo, CO, g, kk, eE, bYz, aoO, Wt, ck, asE, ou, dB, bE, bjx, cj, bC, bt, iu); #c1(LBX1) c2(XP_005269500) c3(transcription factor LBX1 isoform X1) c4(282597) c5(282598, 282599, 282600, 282601, 282602, 282603, 282604) c6( ) c7(T, if, bB, ci, aR, x, Na, aE, arA, asY, rJ, pX, bki, K, pr, aW, lh, bYC, g, cm, ou, iT, bpY, cj, k, m, gU, FG, aj, TZ, nA, qN, axC, jz, aOu, RN, acA, eV, hn, yi, avP, kj, CN, kp, bSZ, kn, kT, YE, Vy, eD, hR, rx, it, B, qc, bL, eX, fZ, aA, v, q, J, boJ, bKE, er, anA, aT, bK, anz, KU, ae, af, bDf, s, vT, NE, il, ev, yL, Lf, r, gm, en, hf, bu, t, xw, cl, bN, amF, ck, iM, c, agw, bt, jh, hF, ad, dP, hd, vJ, ab, nJ, yr, je, qt, ff, nf, gl, aeV, aF, si, bz, dr, bE, bYB, od, VK, X); #c1(LBX2) c2(NP_001009812) c3 (transcription factor LBX2 isoform 2) c4(23694) c5(23695, 23696, 23697, 23698, 23699, 23700) c6( ) c7(Tq); #c1(LBX2) c2(NP_001269359) c3(transcription factor LBX2 isoform 1) c4(101275) c5(101276, 101277, 101278, 101279, 101280, 101281) c6( ) c7(Tq); #c1(LCA5) c2(NP_001116241) c3(Lebercilin) c4(43700) c5(43701, 43702, 43703, 43704, 43705, 43706, 43707, 43708, 43709, 43710) c6( ) c7(alF, nx, bYD, nw, nk, nC, qc); #c1(LCAT) c2(NP_000220) c3(phosphatidylcholine-sterol acyltransferase precursor) c4(4674, 4675, 4676) c5(4677, 4678, 4679, 4680, 4681, 4682, 4683, 4684, 4685, 4686, 4687, 4688, 4689, 4690, 4691, 4692, 4693, 4694, 4695, 4696, 4697, 4698, 4699, 4700, 4701, 4702, 4703, 4704, 4705, 4706, 4707, 4708, 4709, 4710, 4711, 4712, 4713, 4714, 4715, 4716, 4717, 4718, 4719, 4720, 4721, 4722, 4723, 4724, 4725, 4726, 4727, 4728, 4729, 4730, 4731, 4732, 4733, 4734, 4735, 4736, 4737, 4738, 4739, 4740, 4741, 4742, 4743, 4744, 4745, 4746, 4747, 4748, 4749, 4750, 4751, 4752, 4753, 4754, 4755, 4756, 4757, 4758, 4759, 4760, 4761, 4762, 4763, 4764, 4765, 4766, 4767, 4768, 4769, 4770) c6(4771) c7(bL, dD, ef, eR, ea, e, aw, vG, v, j, eq, aY, Zu, nX, dL, dQ, af, hl, Zt, cJ, dr, ek, bYE, mO, dC, cO, at, dy); #c1(LCE1C) c2(NP_001263260) c3(late cornified envelope protein 1C isoform 2) c4( ) c5(97109) c6( ) c7(v, wD, cz); #c1(LCE1C) c2(NP_848128) c3(late cornified envelope protein 1C isoform 1) c4(253745) c5( ) c6( ) c7(v, wD, cz); #c1(LCE3A) c2(NP_848518) c3(late cornified envelope protein 3A) c4(253759) c5( ) c6( ) c7(eE, cr); #c1(LCE3B) c2(NP_848520) c3(late cornified envelope protein 3B) c4(253767) c5( ) c6( ) c7(wD, fJ, cr, eZ, eE, y, blT); #c1(LCE3C) c2(NP_848521) c3(late cornified envelope protein 3C) c4(253768) c5(253769, 253770, 253771, 253772, 253773, 253774) c6( ) c7(pH, wD, fJ, cr, eZ, eE, y, blT); #c1(LCE3D) c2(NP_115952) c3(late cornified envelope protein 3D) c4(224902) c5( ) c6( ) c7(cr, Jr); #c1(LCE3E) c2(NP_848522) c3(late cornified envelope protein 3E) c4(253775) c5( ) c6( ) c7(FO, FP, cr); #c1(LCE5A) c2(NP_848525) c3(late cornified envelope protein 5A) c4(253776) c5(253777) c6( ) c7(eE); #c1(LCK) c2(XP_011539756) c3(tyrosine-protein kinase Lck isoform X1) c4(337970) c5(337971, 337972, 337973, 337974, 337975, 337976) c6( ) c7(T, aCA, dA, bYF, jx, jh, bB, aj, oH, pd, q, J, x, oW, kH, aT, h, hn, aa, A, je, eZ, r, cm, ik, bz, ck, iM, bE, Qu, cj, gZ, k, B); #c1(LCLAT1) c2(NP_001291374) c3(lysocardiolipin acyltransferase 1 isoform 2) c4( ) c5(115783, 115784, 115785, 115786, 115787, 115788, 115789, 115790, 115791, 115792, 115793, 115794, 115795, 115796, 115797) c6( ) c7(LB, fG, cd, fD); #c1(LCLAT1) c2(NP_872357) c3(lysocardiolipin acyltransferase 1 isoform 1) c4(256201, 256202, 256203, 256204, 256205) c5(256206, 256207, 256208, 256209, 256210, 256211, 256212, 256213, 256214, 256215, 256216, 256217, 256218, 256219, 256220, 256221, 256222) c6( ) c7(LB, fG, cd, fD); #c1(LCLAT1) c2(XP_011531047) c3(lysocardiolipin acyltransferase 1 isoform X4) c4( ) c5(327004, 327005, 327006, 327007, 327008, 327009) c6( ) c7(LB, fG, cd, fD); #c1(LCMT1) c2(NP_001027563) c3(leucine carboxyl methyltransferase 1 isoform b) c4(30270) c5(30271, 30272, 30273, 30274, 30275, 30276) c6( ) c7(cm, af, JT); #c1(LCMT1) c2(NP_057393) c3 (leucine carboxyl methyltransferase 1 isoform a) c4(190539) c5(190540, 190541, 190542, 190543, 190544, 190545) c6( ) c7(cm, af, JT); #c1(LCMT1) c2(XP_011544166) c3(leucine carboxy methyltransferase 1 isoform X4) c4(343334) c5(343335, 343336, 343337, 343338, 343339, 343340) c6( ) c7(cm, af, JT); #c1(LCN1) c2(NP_001239546) c3(lipocalin-1 isoform 1 precursor) c4(88508, 88509) c5(88510, 88511, 88512, 88513, 88514, 88515, 88516, 88517, 88518) c6( ) c7(T, b, id, fN, Hn, wR, K, i, af); #c1(LCN1) c2(NP_001239548) c3(lipocalin-1 isoform 3 precursor) c4(88519) c5( ) c6( ) c7(T, b, id, fN, Hn, wR, K, i, af); #c1(LCN1) c2(XP_011516977) c3(lipocalin-1 isoform X1) c4( ) c5(310478) c6( ) c7(T, b, id, fN, Hn, wR, K, i, af); #c1(LCN2) c2(NP_005555) c3(neutrophil gelatinase-associated lipocalin precursor) c4(157425, 157426, 157427, 157428) c5(157429, 157430, 157431, 157432, 157433, 157434, 157435, 157436, 157437, 157438, 157439, 157440, 157441, 157442, 157443, 157444, 157445, 157446, 157447, 157448, 157449, 157450, 157451, 157452, 157453, 157454, 157455, 157456) c6( ) c7(T, aC, aiR, ea, if, al, ci, aY, PB, aR, bmL, aE, K, qM, hu, hm, aa, up, aW, fc, zC, g, zE, axp, nB, wB, U, tV, aUS, m, pw, lS, amz, gD, dD, aw, NT, n, aj, fr, kQ, qN, cd, aDF, Hx, jz, h, lG, ey, A, fQ, aq, fe, kp, pm, ar, Qf, hk, hL, bm, E, B, bL, Ob, dA, eR, cz, cR, v, V, q, eA, eJ, aT, cy, bq, ae, af, aGP, s, il, r, gm, en, cl, LV, t, Ym, ck, sS, kP, c, ac, dh, dy, fD, bt, aZ, wV, eq, atK, ad, fU, hd, afm, sO, oW, Os, ji, lQ, nJ, d, ff, aFt, cr, eZ, iV, bz, bF, D, gk, pc, aS, y, X, BA); #c1(LCOR) c2(NP_001164237) c3(ligand-dependent corepressor isoform 2) c4(66849) c5(66850, 66851, 66852, 66853, 66854) c6( ) c7(v, ae, fc, ad, cP); #c1(LCOR) c2(NP_115816) c3(ligand-dependent corepressor isoform 1) c4(224072) c5(224073, 224074, 224075, 224076, 224077, 224078, 224079, 224080, 224081, 224082, 224083) c6( ) c7(v, ae, fc, ad, cP); #c1(LCOR) c2(XP_006718096) c3(ligand-dependent corepressor isoform X2) c4( ) c5(291504, 291505, 291506, 291507) c6( ) c7(v, ae, fc, ad, cP); #c1(LCP1) c2(XP_005266431) c3(plastin-2 isoform X1) c4(280038, 280039, 280040, 280041) c5(280042, 280043, 280044, 280045, 280046, 280047, 280048, 280049, 280050, 280051, 280052, 280053, 280054, 280055, 280056, 280057, 280058, 280059, 280060, 280061, 280062, 280063, 280064, 280065, 280066, 280067, 280068, 280069) c6( ) c7(bL, T, dD, fM, Zz, ea, e, aw, cz, bB, vG, aj, v, j, ci, eq, q, J, ad, ft, baE, QJ, M, aT, dL, ey, qM, dQ, ae, eL, hm, B, cH, Y, r, fe, iV, t, ck, ar, W, ac, eM, bE, Fi, bP, cj, m, ei, X); #c1(LCP2) c2(NP_005556) c3(lymphocyte cytosolic protein 2) c4(157457) c5(157458, 157459, 157460, 157461, 157462, 157463, 157464, 157465) c6( ) c7(Vv, cy, Cb, zC, Cd); #c1(LCT) c2(NP_002290) c3(lactase-phlorizin hydrolase preproprotein) c4(127353, 127354) c5(127355, 127356, 127357, 127358, 127359, 127360, 127361, 127362, 127363, 127364, 127365, 127366) c6( ) c7(bL, T, dD, e, sM, aiC, aj, v, eq, q, ad, aMb, aT, bCX, bYH, ji, bq, rE, ae, af, bA, aRR, A, r, iV, cm, fq, aMa, aAS, yE, wE, bYG, ei, ra, qc); #c1(LDB1) c2(NP_001106878) c3(LIM domain-binding protein 1 isoform 1) c4(42669) c5(42670, 42671, 42672, 42673, 42674, 42675, 42676, 42677) c6( ) c7(hF, ff, ci, en, pn, aY); #c1(LDB1) c2(NP_003884) c3(LIM domain-binding protein 1 isoform 3) c4(141800) c5(141801, 141802, 141803, 141804, 141805) c6( ) c7(hF, ff, ci, en, pn, aY); #c1(LDB2) c2(NP_001124306) c3(LIM domain-binding protein 2 isoform b) c4(48530) c5(48531, 48532, 48533, 48534, 48535, 48536) c6( ) c7(pn, dD, ac, ea, hF, aw, ff, j, ci, bk, en, eq, aY, fq); #c1(LDB2) c2(NP_001281) c3(LIM domain-binding protein 2 isoform a) c4(111719) c5( ) c6( ) c7(pn, dD, ac, ea, hF, aw, ff, j, ci, bK, en, eq, aY, fq); #c1(LDB2) c2(NP_001291363) c3(LIM domain-binding protein 2 isoform c) c4(115716, 1157(7) c5(115718, 115719, 115720, 115721, 115722, 115723) c6( ) c7(pn, dD, ac, ea, hF, aw, ff, j, ci, bK, en, eq, aY, fq); #c1(LDB2) c2(NP_001291364) c3(LIM domain-binding protein 2 isoform d) c4(115724, 115725, 115726) c5(115727, 115728, 115729, 115730, 115731, 115732, 115733, 115734, 115735, 115736, 115737, 115738, 115739, 115740, 115741, 115742, 115743, 115744, 115745, 115746, 115747, 115748, 115749, 115750, 115751, 115752, 115753, 115754, 115755) c6( ) c7(pn, dD, ac, ea, hF, aw, ff, j, ci, bK, en, eq, aY, fq); #c1(LDB2) c2(XP_006714045) c3(LIM domain-binding protein 2 isoform X8) c4(287517) c5( ) c6( ) c7(pn, dD, ac, ea, hF, aw, ff, j, ci, bK, en, eq, aY, fq); #c1(LDB3) c2(NP_001073583) c3(LIM domain-binding protein 3 isoform 2) c4(36005) c5(36006, 36007, 36008, 36009, 36010, 36011, 36012, 36013, 36014, 36015, 36016, 36017, 36018, 36019, 36020, 36021, 36022, 36023, 36024, 36025, 36026, 36027) c6( ) c7(cc, bfW, Ws, mx, aWn, oj, bt, km, vc, aDB, K, bp, bqW, bYI, bYJ); #c1(LDB3) c2(NP_001073584) c3(LIM domain-binding protein 3 isoform 3) c4(36028) c5(36029, 36030, 36031, 36032, 36033, 36034, 36035, 36036, 36037, 36038, 36039) c6( ) c7(cc, bfW, Ws, mx, aWn, oj, bt, km, vc, aDB, K, bp, bqW, bYI, bYJ); #c1(LDB3) c2(NP_001073585) c3(LIM domain-binding protein 3 isoform 4) c4(36040) c5(36041, 36042, 36043, 36044, 36045, 36046) c6( ) c7(cc, bfW, Ws, mx, aWn, oj, bt, km, vc, aDB, K, bp, bqW, bYI, bYJ); #c1(LDB3) c2(NP_001165081) c3(LIM domain-binding protein 3 isoform 5) c4(67269) c5(67270, 67271, 67272, 67273, 67274, 67275) c6( ) c7(cc, bfW, Ws, mx, aWn, oj, bt, km, vc, aDB, K, bp, bqW, bYI, bYJ); #c1(LDB3) c2(NP_001165082) c3(LIM domain-binding protein 3 isoform 6) c4(67276, 67277) c5(67278, 67279, 67280, 67281, 67282, 67283, 67284) c6( ) c7(cc, bfW, Ws, mx, aWn, oj, bt, km, vc, aDB, K, bp, bqW, bYI, BYJ); #c1(LDB3) c2(NP_009009) c3(LIM domain-binding protein 3 isoform 1) c4(170222) c5( ) c6( ) c7(cc, bfW, Ws, mx, aWn, oj, bt, km, vc, aDB, K, bp, bqW, bYI, bYJ); #c1(LDB3) c2(XP_005269523) c3(LIM domain-binding protein 3 isoform X10) c4(282613) c5(282614, 282615, 282616, 282617, 282618, 282619) c6( ) c7(cc, bfW, Ws, mx, aWn, oj, bt, km, vc, aOB, K, bp, bqW, bYI, bYJ); #c1(LDB3) c2(XP_011537488) c3(LIM domain-binding protein 3 isoform X5) c4(335112) c5( ) c6( ) c7(cc, bfW, Ws, mx, aWn, oj, bt, km, vc, aDB, K, bp, bqW, bYI, bYJ); #c1(LDHA) c2(NP_001128711) c3(L-lactate dehydrogenase A chain isoform 2) c4(50554) c5(50555, 50556, 50557, 50558, 50559, 50560) c6( ) c7(bL, T, kh, aC, aw, jh, aA, bB, n, eb, dG, j, ci, ww, fd, eA, ad, bv, av, cF, py, aE, cc, oj, aT, K, ae, cY, tb, aW, aF, bYK, aB, g, t, cm, iK, ov, es, pc, iA, bP, bE, mx, uS, pg, m, it, B); #c1(LDHA) c2(NP_001158886) c3(L-lactate dehydrogenase A chain isoform 3) c4(63756, 63757) c5(63758, 63759, 63760, 63761, 63762, 63763, 63764, 63765, 63766, 63767, 63768, 63769, 63770, 63771, 63772) c6( ) c7(bL, T, kh, aC, aw, jh, aA, bB, n, eb, dG, j, ci, ww, fd, eA, ad, bv, av, cF, py, aE, cc, oj, aT, K, ae, cY, tb, aW, aF, bYK, aB, g, t, cm, iK, ov, es, pc, iA, bP, bE, mx, uS, pg, m, it, B); #c1(LDHA) c2(NP_001158887) c3(L-lactate dehydrogenase A chain isoform 4) c4(63773, 63774, 63775) c5(63776, 63777, 63778, 63779, 63780, 63781) c6( ) c7(bL, T, kh, aC, aw, jh, aA, bB, n, eb, dG, j, ci, ww, fd, eA, ad, bv, av, cF, py, aE, cc, oj, aT, K, ae, cY, tb, aW, aF, bYK, aB, g, t, cm, ik, ov, es, pc, iA, bP, bE, mx, uS, pg, m, it, B); #c1(LDHA) c2(NP_001158888) c3(L-lactate dehydrogenase A chain isoform 5) c4(63782) c5(63783, 63784, 63785, 63786, 63787, 63788, 63789, 63790, 63791, 63792, 63793, 63794, 63795, 63796, 63797, 63798, 63799, 63800, 63801, 63802, 63803, 63804, 63805, 63806, 63807) c6( ) c7(bL, T, kh, aC, aw, jh, aA, bB, n, eb, dG, j, ci, ww, fd, eA, ad, bv, av, cF, py, aE, cc, oj, aT, K, ae, cY, tb, aW, aF, bYK, aB, g, t, cm, ik, ov, es, pc, iA, bP, bE, mx, uS, pg, m, it, B); #c1(LDHA) c2(NP_005557) c3(L-lactate dehydrogenase A chain isoform 1) c4(157466, 157467, 157468, 157469) c5(157470, 157471, 157472, 157473, 157474, 157475, 157476, 157477, 157478, 157479, 157480, 157481, 157482, 157483, 157484, 157485) c6( ) c7(bL, T, kh, aC, aw, jh, aA, bB, n, eb, dG, j, ci, ww, fd, eA, ad, bv, av, cF, py, aE, cc, oj, aT, K, a, cY, tb, aW, aF, bYK, aB, g, t, cm, ik, ov, es, pc, iA, bP, bE, mx, uS, pg, m, it, B); #c1(LDHAL6B) c2(NP_149972) c3(L-lactate dehydrogenase A-like GB) c4(227674) c5(227675, 227676, 227677, 227678, 227679, 227680) c6( ) c7(aT); #c1(LDHB) c2(NP_002291) c3(L-lactate dehydrogenase B chain) c4( ) c5(127367, 127368, 127369, 127370, 127371, 127372, 127373, 127374, 127375, 127376, 127377, 127378, 127379, 127380, 127381, 127382, 127383, 127384, 127385, 127386, 127387, 127388, 127389, 127390, 127391, 127392) c6( ) c7(bxC, T, eb, bB, n, ww, fr, ad, av, yt, aT, ae, jf, B, tb, aW, aq, g, bz, mO, kP, bE, pg, it, iu); #c1(LDHB) c2(XP_006719137) c3(L-lactate dehydrogenase B chain isoform X1) c4(292220, 292221) c5( ) c6( ) c7(bxC, T, eb, bB, n, ww, fr, ad, av, yt, aT, ae, jf, B, tb, aW, aq, g, bz, mO, kP, bE, pg, it, iu); #c1(LDHC) c2(NP_059144) c3(L-lactate dehydrogenase C chain) c4(193277) c5(193278, 193279, 193280, 193281, 193282, 193283, 193284, 193285, 193286, 193287) c6( ) c7(T, gU, pc, eM, ND, aa); #c1(LDHD) c2(NP_705690) c3(probable D-lactate dehydrogenase, mitochondrial isoform 1 precursor) c4(246182) c5(246183, 246184, 246185, 246186, 246187, 246188) c6( ) c7(bF, T, pa, eG, dA, baE, eM, bE, ev, X, gd, r, bB, dq, ci, bz, q, adn); #c1(LDHD) c2(NP_919417) c3(probable D-lactate dehydrogenase, mitochondrial isoform 2 precursor) c4(258561) c5(258562, 258563, 258564, 258565, 258566, 258567) c6( ) c7(bF, T, pa, eG, dA, baE, eM, bE, ev, X, gd, r, bB, dq, ci, bz, q, adn); #c1(LDLR) c2(NP_000518) c3(low-density lipoprotein receptor isoform 1 precursor) c4(10908, 10909, 10910, 10911, 10912, 10913, 10914, 10915, 10916, 10917, 10918, 10919, 10920, 10921, 10922, 10923, 10924, 10925, 10926, 10927, 10928) c5(10929, 10930, 10931, 10932, 10933, 10934, 10935, 10936, 10937, 10938, 10939, 10940, 10941, 10942, 10943, 10944, 10945, 10946, 10947, 10948, 10949, 10950, 10951, 10952, 10953, 10954, 10955, 10956, 10957, 10958, 10959, 10960, 10961, 10962, 10963, 10964, 10965, 10966, 10967, 10968, 10969, 10970, 10971, 10972, 10973, 10974, 10975, 10976, 10977, 10978, 10979, 10980, 10981, 10982, 10983, 10984, 10985, 10986, 10987, 10988, 10989, 10990, 10991, 10992, 10993, 10994, 10995, 10996, 10997, 10998, 10999, 11000, 11001, 11002, 11003, 11004, 11005, 11006, 11007, 11008, 11009, 11010, 11011, 11012, 11013, 11014, 11015, 11016, 11017, 11018, 11019, 11020, 11021, 11022, 11023, 11024, 11025, 11026, 11027, 11028, 11029, 11030, 11031, 11032, 11033, 11034, 11035, 11036, 11037, 11038, 11039, 11040, 11041, 11042, 11043, 11044, 11045, 11046, 11047, 11048, 11049, 11050, 11051, 11052, 11053, 11054, 11055, 11056, 11057, 11058, 11059, 11060, 11061, 11062, 11063, 11064, 11065, 11066, 11067, 11068, 11069, 11070, 11071, 11072, 11073, 11074, 11075, 11076, 11077, 11078, 11079, 11080, 11081, 11082, 11083, 11084, 11085, 11086, 11087, 11088, 11089, 11090, 11091, 11092, 11093, 11094, 11095, 11096, 11097, 11098, 11099, 11100, 11101, 11102, 11103, 11104, 11105, 11106, 11107, 11108, 11109, 11110, 11111, 11112, 11113, 11114, 11115, 11116, 11117, 11118, 11119, 11120, 11121, 11122, 11123, 11124, 11125, 11126, 11127, 11128, 11129, 11130, 11131, 11132, 11133, 11134, 11135, 11136, 11137, 11138, 11139, 11140, 11141, 11142, 11143, 11144, 11145, 11146, 11147, 11148, 11149, 11150, 11151, 11152, 11153, 11154, 11155, 11156, 11157, 11158, 11159, 11160, 11161, 11162, 11163, 11164, 11165, 11166, 11167, 11168, 11169, 11170, 11171, 11172, 11173, 11174, 11175, 11176, 11177, 11178, 11179, 11180, 11181, 11182, 11183, 11184, 11185, 11186, 11187, 11188, 11189, 11190, 11191, 11192, 11193, 11194, 11195, 11196, 11197, 11198, 11199, 11200, 11201, 11202, 11203, 11204, 11205, 11206, 11207, 11208, 11209, 11210, 11211, 11212, 11213, 11214, 11215, 11216, 11217, 11218, 11219, 11220, 11221, 11222, 11223, 11224, 11225, 11226, 11227, 11228, 11229, 11230, 11231, 11232, 11233, 11234, 11235, 11236, 11237, 11238, 11239, 11240, 11241, 11242, 11243, 11244, 11245, 11246, 11247, 11248, 11249, 11250, 11251, 11252, 11253, 11254, 11255, 11256, 11257, 11258, 11259, 11260, 11261, 11262, 11263, 11264, 11265, 11266, 11267, 11268, 11269, 11270, 11271, 11272, 11273, 11274, 11275, 11276, 11277, 11278, 11279, 11280, 11281, 11282, 11283, 11284, 11285, 11286, 11287, 11288, 11289, 11290, 11291, 11292, 11293, 11294, 11295, 11296, 11297, 11298, 11299, 11300, 11301, 11302, 11303, 11304, 11305, 11306, 11307, 11308, 11309, 11310, 11311, 11312, 11313, 11314, 11315, 11316, 11317, 11318, 11319, 11320, 11321, 11322, 11323, 11324, 11325, 11326, 11327, 11328, 11329, 11330, 11331, 11332, 11333, 11334, 11335, 11336, 11337, 11338, 11339, 11340, 11341, 11342, 11343, 11344, 11345, 11346, 11347, 11348, 11349, 11350, 11351, 11352, 11353, 11354, 11355, 11356, 11357, 11358, 11359, 11360, 11361, 11362, 11363, 11364, 11365, 11366, 11367, 11368, 11369, 11370, 11371, 11372, 11373, 11374, 11375, 11376, 11377, 11378, 11379, 11380, 11381, 11382, 11383, 11384, 11385, 11386, 11387, 11388, 11389, 11390, 11391, 11392, 11393, 11394, 11395, 11396, 11397, 11398, 11399, 11400, 11401, 11402, 11403, 11404, 11405, 11406, 11407, 11408, 11409, 11410, 11411, 11412, 11413, 11414, 11415, 11416, 11417, 11418, 11419, 11420, 11421, 11422, 11423, 11424, 11425, 11426, 11427, 11428, 11429, 11430, 11431, 11432, 11433, 11434, 11435, 11436, 11437, 11438, 11439, 11440, 11441, 11442, 11443, 11444, 11445, 11446, 11447, 11448, 11449, 11450, 11451, 11452, 11453, 11454, 11455, 11456, 11457, 11458, 11459, 11460, 11461, 11462, 11463, 11464, 11465, 11466, 11467, 11468, 11469, 11470, 11471, 11472, 11473, 11474, 11475, 11476, 11477, 11478, 11479, 11480, 11481, 11482, 11483, 11484, 11485, 11486, 11487, 11488, 11489, 11490, 11491, 11492, 11493, 11494, 11495, 11496, 11497, 11498, 11499, 11500, 11501, 11502, 11503, 11504, 11505, 11506, 11507, 11508, 11509, 11510, 11511, 11512, 11513, 11514, 11515, 11516, 11517, 11518, 11519, 11520, 11521, 11522, 11523, 11524, 11525, 11526, 11527, 11528, 11529, 11530, 11531, 11532, 11533, 11534, 11535, 11536, 11537, 11538, 11539, 11540, 11541, 11542, 11543, 11544, 11545, 11546, 11547, 11548, 11549, 11550, 11551, 11552, 11553, 11554, 11555, 11556, 11557, 11558, 11559) c6(11560, 11561, 11562, 11563, 11564, 11565, 11566) c7(bM, T, qo, fR, Zz, ea, e, bB, ur, aY, bsH, AW, dY, u, iX, ari, M, dV, K, qM, dQ, H, BYL, eG, dH, aW, fc, bYP, nY, FU, cm, bw, zV, y, dc, Gs, U, lS, vF, Vj, GV, dD, Ly, wh, aw, aj, PJ, aLc, nV, ec, lt, bYO, h, lG, jy, dL, Go, A, mR, aq, bYR, vo, cM, bbT, aPt, KT, dC, NL, Fi, at, el, oh, fl, B, ek, L, bL, ZV, rG, eR, jx, cz, wo, v, j, bX, so, q, J, eA, ld, aT, bYN, bq, af, bA, bf, il, r, lo, sk, ck, cl, ac, c, au, dh, hr, fP, cS, gT, bt, Kd, nZ, qb, dM, HM, vG, auq, uu, eq, bNM, XM, sg, mC, ad, l, F, ds, bYM, aak, bYQ, d, azS, cH, cW, eZ, dq, dr, qa, ek, bE, bbY, ZD, nT, aS, cP, ei, hE, jr); #c1(LDLR) c2(NP_001182727) c3(low-density lipoprotein receptor isoform 2 precursor) c4(75172) c5(75173, 75174, 75175, 75176, 75177, 75178, 75179) c6( ) c7(bM, T, qo, fR, Zz, ea, e, bB, ur, aY, bsH, AW, dY, u, iX, ari, M, dV, K, qM, dQ, H, bYL, eG, dH, aW, fc, bYP, nY, FU, cm, bw, zV, y, dc, Gs, U, lS, vF, Vj, GV, dD, Ly, wh, aw, aj, PJ, aLc, nV, ec, lt, bYO, h, lG, jy, dL, Go, A, mR, aq, bYR, vo, cM, bbT, aPt, KT, dC, NL, Fi, at, el, oh, fL, B, ek, L, bL, ZV, rG, eR, jx, cz, wo, v, j, bX, so, q, J, eA, ld, aT, bYN, bq, af, bA, bf, il, r, lo, sk, ck, cl, ac, c, au, dh, hr, fP, cS, gT, bt, Kd, nZ, qb, dM, HM, vG, auq, uu, eq, bNM, XM, sg, mC, ad, l, F, ds, bYM, aak, bYQ, d, azS, cH, cW, eZ, dq, dr, qa, ek, bE, bbY, ZD, nT, aS, cP, ei, hE, jr); #c1(LDLR) c2(NP_001182728) c3(low-density lipoprotein receptor isoform 3 precursor) c4(75180, 75181) c5(75182, 75183, 75184, 75185, 75186, 75187, 75188, 75189, 75190, 75191, 75192, 75193, 75194, 75195) c6( ) c7(bM, T, qo, fR, Zz, ea, e, bB, ur, aY, bsH, AW, dY, u, iX, ari, M, dV, K, qM, dQ, H, BYL, eG, dH, aW, fc, BYP, nY, FU, cm, bw, zV, y, dc, Gs, U, lS, vF, Vj, GV, dD, Ly, wh, aw, aj, PJ, aLc, nV, ec, lt, bYO, h, lG, jy, dL, Go, A, mR, aq, bYR, vo, cM, bbT, aPt, kT, dC, NL, Fi, at, el, oh, fL, B, ek, L, bL, ZV, rG, eR, jx, cz, wo, v, j, bX, so, q, J, eA, ld, aT, bYN, bq, af, bA, bf, ill, r, lo, sk, ck, cl, ac, c, au, dh, hr, fP, cS, gT, bt, Kd, nZ, qb, dM, HM, vG, auq, uu, eq, bNM, XM, sg, mC, ad, l, F, ds, bYM, aak, bYQ, d, azS, cH, cW, eZ, dq, dr, qa, ek, bE, bbY, ZD, nT, aS, cP, ei, hE, jr); #c1(LDLR) c2(NP_001182729) c3(low-density lipoprotein receptor isoform 4 precursor) c4(75196, 75197, 75198, 75199, 75200, 75201) c5(75202, 75203, 75204, 75205, 75206, 75207, 75208) c6( ) c7(bM, T, qo, fR, Zz, ea, e, bB, ur, aY, bsH, AW, dY, u, iX, ari, M, dV, K, qM, dQ, H, BYL, eG, dH, aW, fc, bYP, nY, FU, cm, bw, zV, y, dc, Gs, U, lS, vF, Vj, GV, dD, Ly, wh, aw, aj, PJ, aLc, nV, ec, lt, bYO, h, lG, jy, dL, Go, A, mR, aq, bYR, vo, cM, bbT, aPt, kT, dC, NL, Fi, at, el, oh, fl, B, ek, L, bL, ZV, rG, eR, jx, cz, wo, v, j, bX, so, q, J, eA, ld, aT, bYN, bq, af, bA, bf, iU, r, lo, sk, ck, cl, ac, c, au, dh, hr, fP, cS, gT, bt, Kd, nZ, qb, dM, HM, vG, auq, uu, eq, bNM, XM, sg, mC, ad, l, F, ds, bYM, aak, bYQ, d, azS, cH, cW, eZ, dq, dr, qa, ek, bE, bbY, ZD, nT, aS, cP, ei, hE, jr); #c1(LDLR) c2(NP_001182732) c3(low-density lipoprotein receptor isoform 6 precursor) c4(75209) c5(75210, 75211, 75212, 75213, 75214, 75215, 75216) c6( ) c7(bM, T, qo, fR, Zz, ea, e, bB, ur, aY, bsH, AW, dY, u, iX, ari, M, dV, K, qM, dQ, H, bYL, eG, dH, aW, fc, bYP, nY, FU, cm, bw, zV, y, dc, Gs, U, lS, vF, Vj, GV, dD, Ly, wh, aw, aj, PJ, aLc, nV, ec, lt, bYO, h, lG, jy, dL, Go, A, mR, aq, bYR, vo, cM, bbT, aPt, KT, dC, NL, Fi, at, el, oh, fL, B, ek, L, bL, ZV, rG, eR, jx, cz, wo, v, j, bX, so, q, J, eA, ld, aT, bYN, bq, af, bA, bf, ill, r, lo, sk, ck, cl, ac, c, au, dh, hr, fP, cS, gT, bt, Kd, nZ, qb, dM, HM, vG, auq, uu, eq, bNM, XM, sg, mC, ad, l, F, ds, bYM, aak, bYQ, d, azS, cH, cW, eZ, dq, dr, qa, ek, bE, bbY, ZD, nT, aS, cP, ei, hE, jr); #c1(LDLR) c2(XP_011526313) c3(low-density lipoprotein receptor isoform X2) c4(320605) c5( ) c6( ) c7(bM, T, qo, fR, Zz, ea, e, bB, ur, aY, bsH, AW, dY, u, iX, ari, M, dV, K, qM, dQ, H, bYL, eG, dH, aW, fc, bYP, nY, FU, cm, bw, zV, y, dc, Gs, U, lS, vF, Vj, GV, dD, Ly, wh, aw, aj, PJ, aLc, nV, ec, lt, bYO, h, lG, jy, dL, Go, A, mR, aq, bYR, vo, cM, bbT, aPt, KT, dC, NL, Fi, at, el, oh, fL, B, ek, L, bL, ZV, rG, eR, jx, cz, wo, v, j, bX, so, q, J, eA, ld, aT, bYN, bq, af, bA, bf, iU, r, lo, sk, ck, cl, ac, c, au, dh, hr, fP, cS, gT, bt, Kd, nZ, qb, dM, HM, vG, auq, uu, eq, bNM, XM, sg, mC, ad, l, F, ds, bYM, aak, bYQ, d, azS, cH, cW, eZ, dq, dr, qa, ek, bE, bbY, ZD, nT, aS, cP, ei, hE, jr); #c1(LDLRAD3) c2(NP_777562) c3(low-density lipoprotein receptor class A domain-containing protein 3 precursor) c4(251136, 251137) c5(251138, 251139, 251140, 251141, 251142, 251143, 251144, 251145, 251146, 251147, 251148, 251149) c6( ) c7(IB, bN); #c1(LDLRAD4) c2(NP_001003674) c3(low-density lipoprotein receptor class A domain-containing protein 4 isoform gamma 1) c4(19852, 19853) c5(19854, 19855, 19856, 19857, 19858, 19859, 19860, 19861, 19862, 19863) c6( ) c7(fq, an, Ea, cz); #c1(LDLRAD4) c2(NP_001003675) c3(low-density lipoprotein receptor class A domain-containing protein 4 isoform gamma 2) c4(19864, 19865) c5(19866, 19867, 19868, 19869, 19870, 19871, 19872, 19873, 19874, 19875, 19876, 19877) c6( ) c7(fq, an, Ea, cz); #c1(LDLRAD4) c2(NP_852147) c3(low-density lipoprotein receptor class A domain-containing protein 4 isoform alpha 2) c4(254924) c5(254925, 254926, 254927, 254928, 254929, 254930) c6( ) c7(fq, an, Ea, cz); #c1(LDLRAD4) c2(XP_006722416) c3(low-density lipoprotein receptor class A domain-containing protein 4 isoform X2) c4(294704) c5(294705, 294706, 294707, 294708, 294709, 294710) c6( ) c7(fq, an, Ea, cz); #c1(LDLRAD4) c2(XP_011524039) c3(low-density lipoprotein receptor class A domain-containing protein 4 isoform X3) c4( ) c5(318636, 318637, 318638, 318639, 318640, 318641) c6( ) c7(fq, an, Ea, cz); #c1(LDOC1) c2(NP_036449) c3(protein LDOC1) c4(173946) c5(173947, 173948, 173949, 173950, 173951, 173952, 173953) c6( ) c7(T, aC, aE, ff, cz, cj, en); #c1(LDOC1L) c2(NP_115663) c3(protein LDOC1L) c4(223491) c5(223492, 223493, 223494, 223495, 223496, 223497) c6( ) c7(lB); #c1(LEAP2) c2(NP_443203) c3(liver-expressed antimicrobial peptide 2 precursor) c4(229892, 229893, 229894, 229895, 229896, 229897, 229898, 229899, 229900, 229901, 229902, 229903, 229904, 229905) c5(229906, 229907, 229908, 229909, 229910, 229911, 229912) c6(229913) c7(00, h); #c1(LECT1) c2(NP_001011705) c3(leukocyte cell-derived chemotaxin 1 isoform 2 precursor) c4(24759) c5(24760, 24761, 24762, 24763, 24764) c6( ) c7(T, kh, y, up, Qg, n, m, t, dx); #c1(LECT1) c2(NP_008946) c3(leukocyte cell-derived chemotaxin 1 isoform 1 precursor) c4(169711) c5(169712, 169713, 169714, 169715, 169716) c6( ) c7(T, kh, y, up, Qg, n, m, t, dx); #c1(LECT1) c2(XP_006719824) c3(leukocyte cell-derived chemotaxin 1 isoform X5) c4(292994, 292995) c5(292996, 292997, 292998, 292999, 293000, 293001, 293002, 293003, 293004, 293005, 293006, 293007) c6( ) c7(T, kh, y, up, Qg, n, m, t, dx); #c1(LECT1) c2(XP_011533199) c3(leukocyte cell-derived chemotaxin 1 isoform X1) c4( ) c5(329336) c6( ) c7(T, kh, y, up, Qg, n, m, t, dx); #c1(LECT1) c2(XP_011533200) c3(leukocyte cell-derived chemotaxin 1 isoform X2) c4( ) c5(329337) c6( ) c7(T, kh, y, up, Qg, n, m, t, dx); #c1(LECT2) c2(NP_002293) c3(leukocyte cell-derived chemotaxin-2 precursor) c4(127393) c5(127394, 127395, 127396, 127397, 127398, 127399) c6( ) c7(fU, D, iO, jy, aq, v, R, y, B, adz); #c1(LEF1) c2(NP_001124185) c3(lymphoid enhancer-binding factor 1 isoform 2) c4(48454) c5(48455, 48456, 48457, 48458, 48459, 48460) c6( ) c7(gh, gV, T, DE, OD, aA, bB, jh, afK, ci, bYS, il, hF, q, ad, hX, x, jz, aE, bjA, oC, aT, oF, hn, K, jj, aEL, ae, de, hG, aW, Y, oV, eZ, r, gm, af, hf, Z, cl, bw, bz, LB, Cv, ck, apM, bF, bE, X, hL, cj, pg, k, bD, hE, bt, B); #c1(LEF1) c2(NP_001124186) c3(lymphoid enhancer-binding factor 1 isoform 3) c4(48461) c5(48462, 48463, 48464, 48465, 48466, 48467) c6( ) c7(gh, gV, T, DE, OD, aA, bB, jh, afK, ci, bYS, il, hF, q, ad, hX, x, jz, aE, bjA, oC, aT, oF, hn, K, jj, aEL, ae, De, hG, aW, Y, oV, eZ, r, gm, aF, hf, Z, c1, bw, bz, LB, Cv, ck, apM, bF, bE, X, hL, cj, pg, k, bD, hE, bt, B); #c1(LEF1) c2(NP_001159591) c3(lymphoid enhancer-binding factor I isoform 4) c4(64426) c5(64427, 64428, 64429, 64430, 64431, 64432) c6( ) c7(gh, gV, T, DE, OD, aA, bB, jh, afK, ci, bYS, il, hF, q, ad, hX, x, jz, aE, bjA, oC, aT, oF, hn, K, jj, aEL, ae, De, hG, aW, Y, oV, eZ, r, gm, aF, hf, Z, cl, bw, bz, LB, Cv, ck, apM, bF, bE, X, hL, cj, pg, k, bD, hE, bt, B); #c1(LEF1) c2(NP_057353) c3(lymphoid enhancer-binding factor 1 isoform 1) c4(190297) c5(190298, 190299, 190300, 190301, 190302, 190303, 190304) c6( ) c7(gh, gV, T, DE, OD, aA, bB, jh, afK, ci, bYS, il, hF, q, ad, hX, x, jz, aE, bjA, oC, aT, oF, hn, K, jj, aEL, ae, De, hG, aW, Y, oV, eZ, r, gm, aF, hf, Z, cl, bw, bz, LB, Cv, ck, apM, bF, bE, X, hL, cj, pg, k, bD, hE, bt, B); #c1(LEFTY1) c2(NP_066277) c3(left-right determination factor 1 preproprotein) c4(206931) c5(206932, 206933, 206934, 206935, 206936, 206937) c6( ) c7(T, aT); #c1(LEFTY2) c2(NP_001165896) c3(left-right determination factor 2 isoform 2 preproprotein) c4(67959) c5(67960, 67961, 67962, 67963, 67964, 67965, 67966) c6( ) c7(dP, BS, eD, atS, Au, g); #c1(LEFTY2) c2(NP_003231) c3(left-right determination factor 2 isoform 1 preproprotein) c4(136631) c5(136632, 136633, 136634, 136635, 136636, 136637) c6( ) c7(dP, BS, eD, atS, Au, g); #c1(LEKR1) c2(NP_001004316) c3(leucine-, glutamate- and lysine-rich protein 1 isoform 1) c4(20553) c5(20554, 20555, 20556, 20557, 20558) c6( ) c7(V, an); #c1(LEKR1) c2(NP_001180212) c3(leucine-, glutamate- and lysine-rich protein 1 isoform 2) c4( ) c5(73425, 73426, 73427, 73428, 73429) c6( ) c7(V, an); #c1(LEKR1) c2(XP_006713694) c3(leucine-, glutamate- and lysine-rich protein 1 isoform X3) c4( ) c5(287308) c6( ) c7(V, an); #c1(LEKR1) c2(XP_011511107) c3(leucine-, glutamate- and lysine-rich protein 1 isoform X6) c4(301516) c5( ) c6( ) c7(V, an); #c1(LEKR1) c2(XP_011511116) c3(leucine-, glutamate- and lysine-rich protein 1 isoform X12) c4( ) c5(301517, 301518, 301519, 301520, 301521, 301522) c6( ) c7(V, an); #c1(LEKR1) c2(XP_011511118) c3(leucine-, glutamate- and lysine-rich protein 1 isoform X14) c4(301523) c5(301524) c6( ) c7(V, an); #c1(LEMD2) c2(NP_001137416) c3(LEM domain-containing protein 2 isoform 2) c4( ) c5(55655, 55656, 55657, 55658, 55659, 55660, 55661, 55662, 55663, 55664) c6( ) c7(eZ); #c1(LEMD2) c2(NP_851853) c3(LEM domain-containing protein 2 isoform 1) c4(254690, 254691, 254692) c5(254693, 254694, 254695, 254696, 254697, 254698, 254699, 254700) c6( ) c7(eZ); #c1(LEMD3) c2(NP_055134) c3(inner nuclear membrane protein Man1 isoform 1) c4(179195) c5(179196, 179197, 179198, 179199, 179200, 179201) c6( ) c7(bYT, zw, blF, ln, bYU, blO, lQ, MY, aSG); #c1(LEO1) c2(NP_001273359) c3(RNA polymerase-associated protein LEO1 isoform 2) c4(104755) c5(104756, 104757, 104758, 104759, 104760, 104761) c6( ) c7(bt); #c1(LEO1) c2(NP_620147) c3(RNA polymerase-associated protein LEO1 isoform 1) c4(237353, 237354) c5(237355, 237356, 237357, 237358, 237359, 237360, 237361, 237362, 237363, 237364, 237365, 237366) c6( ) c7(bt); #c1(LEP) c2(NP_000221) c3(leptin precursor) c4(4772) c5(4773, 4774, 4775, 4776, 4777, 4778, 4779, 4780) c6( ) c7(gh, T, bM, UH, bQl, fR, tQ, pf, ea, e, al, eb, bB, fd, gf, aoj, ci, bh, Lw, aY, GK, x, Bc, Fo, iX, K, qM, wt, dQ, H, blc, hH, bc, DY, yo, zQ, ZH, aW, az, hl, fc, kt, vs, g, ve, uC, DL, aiv, GR, cm, eM, iA, vA, ZT, Gs, iS, azg, k, m, Uw, GV, dD, tp, lo, n, aj, afE, ct, ml, bjl, fr, zP, aje, cd, ai, rc, hw, h, lG, jy, dL, wo, eV, uc, is, he, alY, ajV, Dg, mR, CN, A, Rj, fQ, aq, fe, zZ, iN, bJ, hq, kT, ok, bP, dB, xO, anZ, Fi, afc, mg, hj, bYW, hL, hR, at, bm, jd, B, L, bL, jv, fM, dA, AL, eR, jx, qQ, eX, Nt, bBX, aA, cz, v, j, bYV, C, sl, hz, nS, V, AO, q, J, btx, UE, eA, mj, aHu, bQG, cF, mP, rS, MP, aql, HC, aT, aHs, cy, bK, bq, bau, ae, af, dl, bA, qw, bSl, kl, bf, s, aGP, il, oV, Y, fO, bSm, r, iy, en, hf, cl, t, f, bQ, Dm, bN, ck, Qi, rR, nM, tW, c, ac, au, gX, hr, mf, cS, dy, Da, UF, gT, bt, yv, gx, sY, tS, aZ, aD, iW, jN, jh, bS, dG, ah, eq, hF, ad, afb, qC, Jm, dP, av, LL, F, QJ, ds, oW, aZW, Dj, nJ, sj, d, cH, baT, xs, ff, zR, cr, eZ, gl, dq, aF, Tp, iV, Z, FX, iR, bz, dr, qa, dR, bF, es, D, OG, bE, pE, gZ, aS, cP, ei, pk, hE, X, y); #c1(LEPROTL1) c2(NP_001121680) c3(leptin receptor overlapping transcript-like 1 isoform 2) c4(46034) c5(46035, 46036, 46037, 46038, 46039, 46040) c6( ) c7(v, ey); #c1(LEPROTL1) c2(NP_056159) c3(leptin receptor overlapping transcript-like 1 isoform 1) c4(186022) c5(186023, 186024, 186025, 186026, 186027, 186028) c6( ) c7(v, ey); #c1(LETM1) c2(NP_036450) c3(LETM1 and EF-hand domain-containing protein 1, mitochondrial precursor) c4(173954, 173955) c5(173956, 173957, 173958, 173959, 173960, 173961, 173962) c6( ) c7(av, yf, eM, ha, Az, amj, Ko, nA, bz, B, axC); #c1(LETM1) c2(XP_006713947) c3(LETM1 and EF-hand domain-containing protein 1, mitochondrial isoform X1) c4( ) c5(287429, 287430, 287431, 287432, 287433) c6( ) c7(av, yf, eM, ha, Az, amj, Ko, nA, bz, B, axC); #c1(LETMD1) c2(NP_001230618) c3(LETM1 domain-containing protein 1 isoform 3) c4(86401) c5(86402, 86403, 86404, 86405, 86406, 86407) c6( ) c7(ck, T, aW, if, r, aT, ci, qN, eA, q); #c1(LETMD1) c2(NP_001287694) c3(LETM1 domain-containing protein 1 isoform 7) c4(112569) c5(112570, 112571, 112572, 112573, 112574) c6( ) c7(ck, T, aW, if, r, aT, ci, qN, eA, q); #c1(LETMD1) c2(NP_056231) c3(LETM1 domain-containing protein 1 isoform 1) c4(186608, 186609) c5(186610, 186611, 186612, 186613, 186614, 186615, 186616, 186617, 186618, 186619, 186620, 186621) c6( ) c7(ck, T, aW, if, r, aT, ci, qN, eA, q); #c1(LETMD1) c2(XP_006719398) c3(LETM1 domain-containing protein 1 isoform X5) c4(292458) c5(292459, 292460, 292461, 292462, 292463, 292464, 292465) c6( ) c7(ck, T, aW, if, r, aT, ci, qN, eA, q); #c1(LEUTX) c2(NP_001137304) c3(leucine-twenty homeobox) c4(55416, 55417) c5(55418, 55419, 55420, 55421, 55422, 55423, 55424, 55425, 55426, 55427, 55428, 55429) c6( ) c7(bt); #c1(LFNG) c2(NP_001035257) c3(beta-1,3-N-acetylglucosaminyltransferase lunatic fringe isoform a preproprotein) c4(32687) c5(32688, 32689, 32690, 32691, 32692, 32693) c6( ) c7(bYX, Al, aT); #c1(LFNG) c2(NP_001035258) c3(beta-1,3-N-acetylglucosaminyltransferase lunatic fringe isoform b preproprotein) c4(32694) c5(32695, 32696, 32697, 32698, 32699, 32700) c6( ) c7(bYX, Al, aT); #c1(LFNG) c2(NP_001159827) c3(beta-1,3-N-acetylglucosaminyltransferase lunatic fringe isoform c) c4(64801) c5(64802, 64803, 64804, 64805, 64806, 64807, 64808, 64809, 64810) c6( ) c7(bYX, Al, aT); #c1(LFNG) c2(NP_002295) c3(beta-1,3-N-acetylglucosaminyltransferase lunatic fringe isoform d) c4(127400) c5(127401, 127402, 127403, 127404, 127405, 127406, 127407, 127408, 127409, 127410) c6( ) c7(bYX, Al, aT); #c1(LGALS1) c2(NP_002296) c3(galectin-1) c4(127411) c5(127412, 127413, 127414, 127415, 127416, 127417) c6( ) c7(bM, T, if, eb, bB, fd, ci, xE, aE, il, fm, rJ, DO, K, pk, eL, DA, aW, hJ, cm, uC, eM, Hb, m, ul, n, aj, uE, qN, ai, eW, jz, h, eV, dL, Ha, kp, jT, cM, auC, ok, dB, mg, B, fM, fG, Ju, eX, aA, hz, C, q, eA, aT, ae, hM, arl, xR, oV, Y, r, lo, en, t, ik, ck, nM, c, id, iW, jh, Fc, ad, hd, py, Ps, nJ, d, ff, cr, aF, VI, bz, bE, Hs, hE, iu, y); #c1(LGALS12) c2(NP_001136007) c3(galectin-12 isoform 1) c4(53986) c5(53987, 53988, 53989, 53990, 53991, 53992) c6( ) c7(T, eM); #c1(LGALS12) c2(NP_001136008) c3(galectin-12 isoform 3) c4(53993) c5(53994, 53995, 53996, 53997, 53998, 53999, 54000) c6( ) c7(T, eM); #c1(LGALS12) c2(NP_001136009) c3(galectin-12 isoform 4) c4( ) c5(54001, 54002, 54003, 54004, 54005) c6( ) c7(T, eM); #c1(LGALS12) c2(NP_001136010) c3(galectin-12 isoform 5) c4(54006) c5(54007, 54008, 54009, 54010, 54011) c6( ) c7(T, eM); #c1(LGALS12) c2(NP_149092) c3(galectin-12 isoform 2) c4(227094, 227095) c5(227096, 227097, 227098, 227099, 227100, 227101, 227102) c6( ) c7(T, eM); #c1(LGALS13) c2(NP_037400) c3(galactoside-binding soluble lectin 13) c4(175295) c5(175296, 175297, 175298, 175299, 175300, 175301) c6( ) c7(nJ, gx, T, hd, B, Tb, AL, Tg, bE, jz, bB, aq, uC, ae, cd, ad); #c1(LGALS14) c2(NP_064514) c3(placental protein 13-like isoform 1) c4(202824) c5(202825, 202826, 202827, 202828, 202829, 202830) c6( ) c7(Cq, aPn, ac, e, Ji, aw, aCs, ao, j, hg, ji, iV, cb, eu, eA, aR); #c1(LGALS14) c2(NP_982297) c3(placental protein 13-like isoform 2) c4(263308) c5(263309, 263310, 263311, 263312, 263313, 263314) c6( ) c7(Cq, aPn, ac, e, Ji, aw, aCs, ao, j, hg, ji, iV, cb, eu, eA, aR); #c1(LGALS16) c2(NP_001177370) c3(galectin-16) c4(72709) c5(72710, 72711, 72712, 72713, 72714, 72715) c6( ) c7(Tg, T, hd, B, Tb, jz, aq, cd); #c1(LGALS2) c2(NP_006489) c3(galectin-2) c4(165637, 165638) c5(165639, 165640, 165641, 165642, 165643, 165644, 165645, 165646, 165647, 165648, 165649, 165650) c6( ) c7(cT, ac, eR, e, eh, aw, M, aA, cz, dV, wz, aF, j, ci, ei, fe, ji, y, aDi, eu, eK); #c1(LGALS3) c2(NP_001170859) c3(galectin-3 isoform 2) c4(69566) c5(69567) c6( ) c7(bM, T, aC, Tb, FR, ea, if, e, al, bB, ci, ip, AW, xE, aE, M, K, hu, eL, cp, yo, aW, bvD, Du, hl, g, as, cm, qJ, eM, nB, cj, m, dD, lT, aw, ht, yG, n, aj, fr, uE, qN, cd, eW, ft, jz, cc, gd, dL, A, PX, AQ, fQ, aq, fe, ajz, dB, hk, hR, E, B, AN, vQ, fM, AS, fG, lH, yk, fZ, aA, cz, hz, v, j, q, J, eA, qK, aT, cy, Fx, jj, bq, ae, eu, Uf, s, NE, iU, Y, ev, fO, r, iK, pb, ck, bnc, cT, sS, nM, ac, c, iM, aJQ, bt, Mj, id, arh, KO, eq, ad, hX, av, Tg, hd, ib, Db, oj, bYY, d, je, eZ, af, iV, bz, Hp, LB, bE, lF, HE, X, y); #c1(LGALS3) c2(XP_011535061) c3(galectin-3 isoform X1) c4(331726, 331727) c5(331728, 331729, 331730, 331731, 331732, 331733, 331734, 331735, 331736, 331737, 331738, 331739, 331740, 331741, 331742, 331743, 331744, 331745, 331746, 331747, 331748, 331749, 331750, 331751, 331752, 331753, 331754, 331755, 331756, 331757, 331758, 331759, 331760, 331761, 331762, 331763, 331764, 331765, 331766, 331767, 331768, 331769, 331770, 331771, 331772, 331773, 331774, 331775, 331776, 331777, 331778, 331779, 331780, 331781, 331782, 331783, 331784, 331785, 331786, 331787, 331788, 331789, 331790, 331791, 331792, 331793, 331794, 331795, 331796, 331797, 331798, 331799, 331800, 331801, 331802, 331803, 331804, 331805, 331806, 331807, 331808, 331809, 331810, 331811, 331812, 331813, 331814, 331815, 331816, 331817, 331818, 331819, 331820) c6(331821) c7(bM, T, aC, Tb, FR, ea, if, e, al, bB, ci, ip, AW, xE, aE, M, K, hu, eL, cp, yo, aW, bvD, Du, hl, g, as, cm, qJ, eM, nB, cj, m, dD, lT, aw, ht, yG, n, aj, fr, uE, qN, cd, eW, ft, jz, cc, gd, dL, A, PX, AQ, fQ, aq, fe, ajz, dB, hk, hR, E, B, AN, vQ, fM, AS, fG, lH, yk, fZ, aA, cz, hz, v, j, q, J, eA, qK, aT, cy, Fx, jj, bq, ae, eu, Uf, s, NE, iU, Y, ev, fO, r, iK, pb, ck, bnc, cT, sS, nM, ac, c, iM, aJQ, bt, Mj, id, arh, KO, eq, ad, hX, av, Tg, hd, ib, Db, oj, bYY, d, je, eZ, aF, iV, bz, Hp, LB, bE, lF, hE, X, y); #c1(LGALS3BP) c2(NP_005558) c3(galectin-3-binding protein precursor) c4(157486, 157487) c5(157488, 157489, 157490, 157491, 157492, 157493, 157494, 157495, 157496, 157497, 157498, 157499) c6( ) c7(T, wu, jx, aA, bB, q, ad, av, aT, h, ZQ, wm, ae, aW, ff, r, Ph, aF, en, cm, t, bz, bE, y, X); #c1(LGALS4) c2(NP_006140) c3(galectin-4) c4(162354) c5(162355, 162356, 162357, 162358, 162359, 162360) c6( ) c7(T, jx, n, ci, q, ad, hX, dP, Yn, ds, aT, ae, s, aW, ev, r, g, t, bz, c, m, bt, B); #c1(LGALS7B) c2(NP_001035972) c3(galectin-7) c4(33590) c5(33591, 33592, 33593, 33594, 33595, 33596, 33597, 33598, 33599, 33600, 33601, 33602) c6(33603, 33604) c7(T, b, ajm, bE, sw, Y, aW, if, ff, jh, aT, aA, dL, aF, ci, en, qN, i, cm, el); #c1(LGALS8) c2(NP_006490) c3(galectin-8 isoform a) c4(165651, 165652, 165653, 165654) c5(165655, 165656, 165657, 165658, 165659, 165660, 165661, 165662, 165663, 165664, 165665, 165666, 165667, 165668, 165669, 165670, 165671, 165672, 165673, 165674, 165675, 165676, 165677, 165678) c6( ) c7(ae, T, Ub, gH, Qu, ci, bz, ad); #c1(LGALS8) c2(NP_963838) c3(galectin-8 isoform b) c4(262379, 262380) c5(262381, 262382, 262383, 262384, 262385, 262386, 262387, 262388, 262389, 262390, 262391, 262392, 262393, 262394, 262395, 262396, 262397, 262398, 262399, 262400, 262401, 262402, 262403, 262404, 262405, 262406, 262407, 262408, 262409, 262410, 262411, 262412, 262413, 262414, 262415, 262416, 262417, 262418) c6( ) c7(ae, T, Ub, gH, Qu, ci, bz, ad); #c1(LGALS8) c2(XP_011542490) c3(galectin-8 isoform X1) c4(341013, 341014) c5(341015, 341016, 341017, 341018, 341019, 341020) c6( ) c7(ae, T, Ub, gH, Qu, ci, bz, ad); #c1(LGALS9) c2(NP_002299) c3(galectin-9 isoform short) c4(127418) c5(127419, 127420, 127421, 127422, 127423, 127424) c6( ) c7(gx, Kd, T, ex, aA, bB, fd, jE, ci, uE, bh, lu, zo, q, ai, hX, xE, aT, h, eV, ji, pk, Nr, je, il, aW, Y, ff, bg, dN, po, aF, en, iV, ajz, Nq, ba, iM, Jr, bE, aCl, dW, fD, y, B); #c1(LGALS9) c2(NP_033665) c3(galectin-9 isoform 1ong) c4(172303) c5(172304, 172305, 172306, 172307, 172308, 172309) c6( ) c7(gx, Kd, T, ex, aA, bB, fd, jE, ci, uE, bh, lu, zo, q, ai, hX, xE, aT, h, eV, ji, pk, Nr, je, iU, aW, Y, ff, bg, dN, po, aF, en, iV, ajz, Nq, ba, iM, Jr, bE, aCl, dW, fD, y, B); #c1(LGALS9) c2(XP_006721955) c3(galectin-9 isoform X1) c4(294471) c5(294472, 294473, 294474, 294475, 294476, 294477) c6( ) c7(gx, Kd, T, eX, aA, bB, fd, jE, ci, uE, bh, lu, zo, q, ai, hX, xE, aT, h, eV, ji, pk, Nr, je, il, aW, Y, ff, bg, dN, po, aF, en, iV, ajz, Nq, ba, iM, Jr, bE, aCl, dW, fD, y, B); #c1(LGALS9) c2(XP_006721956) c3(galectin-9 isoform X2) c4(294478) c5(294479, 294480, 294481, 294482, 294483, 294484) c6( ) c7(gx, Kd, T, eX, aA, bB, fd, jE, ci, uE, bh, lu, zo, q, ai, hX, xE, aT, h, eV, ji, pk, Nr, je, iU, aW, Y, ff, bg, dN, po, aF, en, iV, ajz, Nq, ba, iM, Jr, bE, aCl, dW, fD, y, B); #c1(LGALS9) c2(XP_006721957) c3(galectin-9 isoform X3) c4(294485) c5(294486, 294487, 294488, 294489, 294490, 294491) c6( ) c7(gx, Kd, T, ex, aA, bB, fd, jE, ci, uE, bh, lu, zo, q, ai, hX, xE, aT, h, eV, ji, pk, Nr, je, iU, aW, Y, ff, bg, dN, po, aF, en, iV, ajz, Nq, ba, iM, Jr, bE, aCl, dW, fD, y, B); #c1(LGALS9) c2(XP_006721958) c3(galectin-9 isoform X4) c4(294492) c5( ) c6( ) c7(gx, Kd, T, ex, aA, bB, fd, jE, ci, uE, bh, lu, zo, q, ai, hX, xE, aT, h, eV, ji, pk, Nr, je, il, aW, Y, ff, bg, dN, po, aF, en, iV, ajz, Nq, ba, iM, Jr, bE, aCl, dW, fD, y, B); #c1(LGALS9) c2(XP_011523098) c3(galectin-9 isoform X5) c4( ) c5(317610, 317611, 317612, 317613, 317614, 317615) c6( ) c7(gx, Kd, T, ex, aA, bB, fd, jE, ci, uE, bh, lu, zo, q, ai, hX, xE, aT, h, eV, ji, pk, Nr, je, ill, aW, Y, ff, bg, dN, po, aF, en, iV, ajz, Nq, ba, iM, Jr, bE, aCl, dW, fD, y, B); #c1(LGALSL) c2(NP_054900) c3(galectin-related protein) c4(178259) c5(178260, 178261, 178262, 178263, 178264, 178265) c6( ) c7(T, aA, fr, eA, ad, av, ft, zw, aE, pX, aT, K, ae, gj, aW, aF, g, cm, t, bz, ik, es, eM, iA, va, m, y); #c1(LGI1) c2(NP_005088) c3(leucine-rich glioma-inactivated protein 1 precursor) c4(152791) c5(152792, 152793, 152794, 152795, 152796, 152797) c6( ) c7(wY, bZd, oe, ha, lr, bS, gf, ct, bZa, ci, il, eA, aR, yt, hb, bZb, K, hg, bYZ, d, ahJ, as, Z, cm, dr, Hp, Cq, dB, bZc, FJ, avl, it); #c1(LGI2) c2(NP_060646) c3(leucine-rich repeat LGI family member 2 precursor) c4(196966, 196967) c5(196968, 196969, 196970, 196971, 196972, 196973, 196974, 196975, 196976, 196977, 196978, 196979) c6( ) c7(ha, j, lr); #c1(LGI4) c2(NP_644813) c3(leucine-rich repeat LGI family member 4 precursor) c4(238581, 238582, 238583) c5(238584, 238585, 238586, 238587, 238588, 238589, 238590, 238591, 238592, 238593, 238594, 238595, 238596, 238597, 238598, 238599, 238600, 238601) c6( ) c7(HN, Cq, ha, akq); #c1(LGMN) c2(NP_005597) c3(legumain preproprotein) c4(157793, 157794, 157795) c5(157796, 157797, 157798, 157799, 157800, 157801, 157802, 157803, 157804, 157805, 157806, 157807, 157808, 157809, 157810, 157811, 157812, 157813, 157814, 157815, 157816, 157817, 157818, 157819, 157820, 157821, 157822) c6( ) c7(T, dD, ea, aA, n, eq, V, q, ad, ke, Ma, Ot, rJ, cy, K, ae, azh, asp, r, aF, t, FV, FJ, m, ajJ, B); #c1(LGMN) c2(XP_005267919) c3(legumain isoform X2) c4(281289) c5(281290, 281291, 281292, 281293, 281294, 281295) c6( ) c7(T, dD, ea, aA, n, eq, V, q, ad, ke, Ma, Ot, rJ, cy, K, ae, azh, asp, r, aF, t, FV, FJ, m, ajJ, B); #c1(LGMN) c2(XP_005267920) c3(legumain isoform X3) c4(281296, 281297) c5(281298, 281299, 281300, 281301, 281302, 281303, 281304, 281305, 281306, 281307, 281308, 281309, 281310, 281311, 281312, 281313, 281314, 281315, 281316, 281317, 281318, 281319, 281320, 281321, 281322, 281323, 281324, 281325, 281326) c6( ) c7(T, dD, ea, aA, n, eq, V, q, ad, ke, Ma, Ot, rJ, cy, K, ae, azh, asp, r, aF, t, FV, FJ, m, ajJ, B); #c1(LGMN) c2(XP_011535272) c3(legumain isoform X1) c4(332173, 332174, 332175) c5( ) c6( ) c7(T, dD, ea, aA, n, eq, V, q, ad, ke, Ma, Ot, rJ, cy, K, ae, azh, asp, r, aF, t, FV, FJ, m, ajJ, B); #c1(LGR4) c2(NP_060960) c3(leucine-rich repeat-containing G-protein coupled receptor 4 precursor) c4(198888) c5(198889, 198890, 198891, 198892, 198893, 198894) c6( ) c7(T, aA, r, aqK, ko, aF, ci, nA, q); #c1(LGR5) c2(NP_001264155) c3(leucine-rich repeat-containing G-protein coupled receptor 5 isoform 2 precursor) c4(97594) c5(97595, 97596, 97597, 97598, 97599, 97600) c6( ) c7(bL, T, aD, oe, ht, NT, aA, bB, n, aj, v, j, ci, bZe, q, eA, afQ, h, hu, KV, d, B, s, r, hl, aF, g, dB, bE, gX, X); #c1(LGR5) c2(NP_001264156) c3(leucine-rich repeat-containing G-protein coupled receptor 5 isoform 3 precursor) c4(97601) c5(97602, 97603, 97604, 97605, 97606, 97607) c6( ) c7(bL, T, aD, oe, ht, NT, aA, bB, n, aj, v, j, ci, bZe, q, eA, afQ, h, hu, KV, d, B, s, r, hQ, aF, g, dB, bE, gX, X); #c1(LGR5) c2(NP_003658) c3(leucine-rich repeat-containing G-protein coupled receptor 5 isoform 1 precursor) c4(140020, 140021) c5(140022, 140023, 140024, 140025, 140026, 140027, 140028, 140029, 140030, 140031, 140032, 140033) c6( ) c7(bL, T, aD, oe, ht, NT, aA, bB, n, aj, v, j, ci, bZe, q, eA, afQ, h, hu, KV, d, B, s, r, hQ, aF, g, dB, bE, gX, X); #c1(LGR6) c2(NP_001017403) c3 (leucine-rich repeat-containing G-protein coupled receptor 6 isoform 1 precursor) c4(26419, 26420) c5(26421, 26422, 26423, 26424, 26425, 26426, 26427, 26428, 26429, 26430, 26431, 26432) c6( ) c7(bL, bM, gU, bZf, Ja, v, ci, aE, pX, aT, h, he, Fg, avg, d, aW, Y, ff, aeS, baZ, en, cl, bfw, dX, cE); #c1(LGR6) c2(NP_001017404) c3(leucine-rich repeat-containing G-protein coupled receptor 6 isoform 3) c4(26433) c5(26434, 26435, 26436, 26437, 26438, 26439) c6( ) c7(bL, bM, gU, bZf, Ja, v, ci, aE, pX, aT, h, he, Fg, avg, d, aW, Y, ff, aeS, baZ, en, cl, bfw, dX, cE); #c1(LGRG) c2(NP_067649) c3(leucine-rich repeat-containing G-protein coupled receptor 6 isoform 2) c4(209232) c5(209233, 209234, 209235, 209236, 209237, 209238, 209239) c6( ) c7(bL, bM, gU, bZf, Ja, v, ci, aE, pX, aT, h, he, Fg, avg, d, aW, Y, ff, aeS, baZ, en, cl, bfw, dX, cE); #c1(LGR6) c2(XP_011508140) c3(leucine-rich repeat-containing G-protein coupled receptor 6 isoform X2) c4(298633) c5(298634, 298635, 298636, 298637, 298638) c6( ) c7(bL, bM, gU, bZf, Ja, v, ci, aE, pX, aT, h, he, Fg, avg, d, aW, Y, ff, aeS, baZ, en, cl, bfw, dX, cE); #c1(LGSN) c2(NP_001137412) c3(lengsin isoform b) c4(55648) c5(55649, 55650, 55651, 55652, 55653, 55654) c6( ) c7(T, TY, aEX, aw, asM, bz, B); #c1(LGSN) c2(NP_057655) c3(lengsin isoform a) c4(192120) c5(192121, 192122, 192123, 192124, 192125, 192126) c6( ) c7(T, TY, aEX, aw, asM, bz, B); #c1(LHB) c2(NP_000885) c3(lutropin subunit beta precursor) c4(16827) c5(16828, 16829, 16830, 16831, 16832, 16833, 16834, 16835, 16836) c6( ) c7(dP, kl, gU, zZ, il, aec, bZg, aW, aT, cz, UE, avO, UF, pt, af, ad, bN, biN); #c1(LHB) c2(XP_011525277) c3(lutropin subunit beta isoform X1) c4(319718) c5(319719, 319720, 319721, 319722, 319723, 319724, 319725, 319726) c6(319727, 319728) c7(dP, kl, gU, zZ, il, aec, bZg, aW, aT, cz, UE, avO, UF, pt, af, ad, bN, biN); #c1(LHCGR) c2(NP_000224) c3(lutropin-choriogonadotropic hormone receptor precursor) c4(4789, 4790, 4791, 4792) c5(4793, 4794, 4795, 4796, 4797, 4798, 4799, 4800, 4801, 4802, 4803, 4804, 4805, 4806, 4807, 4808, 4809, 4810, 4811, 4812, 4813, 4814, 4815) c6( ) c7(T, UH, gU, eR, wu, aec, zX, aZ, vf, jN, eb, adW, bZf, v, ci, zP, ad, UE, bSU, il, BJ, aT, Ps, bZk, iv, wm, uV, Fg, ae, af, il, alL, aeQ, yi, kl, UL, s, aW, vj, Lf, aeU, aeS, bZj, bbs, Pp, baZ, Hu, bu, cl, t, bN, bZh, bZi, sn, ac, AV, anZ, m); #c1(LHCGR) c2(XP_005264366) c3(lutropin-choriogonadotropic hormone receptor isoform X6) c4( ) c5(278829) c6( ) c7(T, UH, gU, eR, wu, aec, zX, aZ, vf, jN, eb, adW, bZf, v, ci, zP, ad, UE, bSU, il, BJ, aT, Ps, bZk, iv, wm, uV, Fg, ae, af, il, alL, aeQ, yi, kl, UL, s, aW, vj, Lf, aeU, aeS, bZj, bbs, Pp, baZ, Hu, bu, cl, t, bN, bZh, bZi, sn, ac, AV, anZ, m); #c1(LHCGR) c2(XP_011531131) c3(lutropin-choriogonadotropic hormone receptor isoform X2) c4(327110) c5(327111, 327112, 327113, 327114, 327115, 327116) c6( ) c7(T, UH, gU, eR, wu, aec, zX, aZ, vf, jN, eb, adW, bZf, v, ci, zP, ad, UE, bSU, il, BJ, aT, Ps, bZk, iv, wm, uV, Fg, ae, af, il, alL, aeQ, yi, kl, UL, s, aW, vj, Lf, aeU, aeS, bZj, bbs, Pp, baZ, Hu, bu, cl, t, bN, bZh, bZi, sn, ac, AV, anZ, m); #c1(LHFP) c2(XP_011533163) c3(lipoma HMGIC fusion partner isoform X1) c4(329302) c5(329303, 329304, 329305, 329306, 329307, 329308, 329309) c6( ) c7(T, Qf, cr, hj, cz, aHs, vr, j, Xk, eA, On); #c1(LHFPL1) c2(XP_011529245) c3(lipoma HMGIC fusion partner-like 1 protein isoform X2) c4( ) c5(324686) c6( ) c7(vr); #c1(LHFPL1) c2(XP_011529246) c3(lipoma HMGIC fusion partner-like I protein isoform X1) c4(324687) c5(324688, 324689, 324690, 324691, 324692) c6( ) c7(vr); #c1(LHFPL1) c2(XP_011529248) c3(lipoma HMGIC fusion partner-like 1 protein isoform X4) c4(324693) c5(324694, 324695, 324696, 324697, 324698, 324699) c6( ) c7(vr); #c1(LHFPL2) c2(NP_005770) c3(lipoma HMGIC fusion partner-like 2 protein) c4(159214) c5(159215, 159216, 159217, 159218, 159219, 159220, 159221, 159222, 159223, 159224) c6( ) c7(ey); #c1(LHFPL3) c2(NP_945351) c3 (lipoma HMGIC fusion partner-like 3 protein) c4(260865, 260866) c5(260867, 260868, 260869, 260870, 260871, 260872, 260873, 260874, 260875, 260876, 260877, 260878) c6( ) c7(T, d, ac, if, cz, v, qN, bN); #c1(LHFPL3) c2(XP_005250384) c3(lipoma HMGIC fusion partner-like 3 protein isoform X1) c4(270284) c5(270285, 270286, 270287, 270288, 270289, 270290) c6( ) c7(T, d, ac, if, cz, v, qN, bN); #c1(LHFPL4) c2(XP_011531995) c3(lipoma HMGIC fusion partner-like 4 protein isoform X1) c4(328036) c5(328037, 328038, 328039, 328040, 328041, 328042) c6( ) c7(T, if, qN); #c1(LHFPL5) c2(NP_872354) c3(tetraspan membrane protein of hair cell stereocilia) c4(256190) c5(256191, 256192, 256193, 256194, 256195, 256196, 256197, 256198, 256199, 256200) c6( ) c7(bxn, Bh, bZl); #c1(LHPP) c2(NP_001161352) c3(phospholysine phosphohistidine inorganic pyrophosphate phosphatase isoform 2) c4(65624) c5(65625, 65626, 65627, 65628, 65629, 65630) c6( ) c7(bO); #c1(LHPP) c2(NP_071409) c3(phospholysine phosphohistidine inorganic pyrophosphate phosphatase isoform 1) c4(211504) c5(211505, 211506, 211507, 211508, 211509, 211510) c6( ) c7(bO); #c1(LHX1) c2(XP_011545112) c3(LIM/homeobox protein Lhx1 isoform X1) c4(343970) c5(343971, 343972, 343973, 343974, 343975, 343976, 343977) c6( ) c7(bfP, T, bxG, Ur, fr); #c1(LHX2) c2(NP_004780) c3(LIM/homeobox protein Lhx2) c4(149568) c5(149569, 149570, 149571, 149572, 149573, 149574, 149575) c6( ) c7(gk, T, mj, kj, iU, aW, al, oW, aT, y, biN); #c1(LHX3) c2(NP_055379) c3(LIM/homeobox protein Lhx3 isoform b) c4(180672, 180673) c5(180674, 180675, 180676, 180677, 180678, 180679, 180680, 180681, 180682, 180683, 180684, 180685, 180686) c6( ) c7(bBJ, acz, ajX, mG, v, bZm, bHF); #c1(LHX3) c2(NP_835258) c3(LIM/homeobox protein Lhx3 isoform a) c4(253436) c5(253437, 253438, 253439, 253440, 253441, 253442) c6( ) c7(bBJ, acz, ajX, mG, v, bZm, bHF); #c1(LHX4) c2(NP_203129) c3(LIM/homeobox protein Lhx4) c4(228348) c5(228349, 228350, 228351, 228352, 228353, 228354, 228355) c6( ) c7(x, bZn, il, acz, tT, k, bHF, biN); #c1(LHX5) c2(NP_071758) c3(LIM/homeobox protein Lhx5) c4(211971) c5(211972, 211973, 211974, 211975, 211976, 211977, 211978) c6( ) c7(aj); #c1(LHX6) c2(NP_001229262) c3(LIM/homeobox protein Lhx6 isoform 3) c4(84045) c5(84046, 84047, 84048, 84049, 84050, 84051) c6( ) c7(yL, T, bz); #c1(LHX6) c2(NP_001229263) c3(LIM/homeobox protein Lhx6 isoform 4) c4(84052, 84053) c5(84054, 84055, 84056, 84057, 84058, 84059) c6( ) c7(yL, T, bz); #c1(LHX6) c2(NP_001229264) c3(LIM/homeobox protein Lhx6 isoform 5) c4( ) c5(84060, 84061, 84062, 84063, 84064) c6( ) c7(yL, T, bz); #c1(LHX6) c2(NP_055183) c3(LIM/homeobox protein Lhx6 isoform 1) c4(179637) c5(179638, 179639, 179640, 179641, 179642, 179643, 179644, 179645) c6( ) c7(yL, T, bz); #c1(LHX6) c2(NP_954629) c3(LIM/homeobox protein Lhx6 isoform 2) c4(261086) c5(261087, 261088, 261089, 261090, 261091, 261092) c6( ) c7(yL, T, bz); #c1(LHX6) c2(XP_005251973) c3(LIM/homeobox protein Lhx6 isoform X4) c4( ) c5(271380, 271381, 271382, 271383, 271384, 271385, 271386, 271387, 271388, 271389, 271390, 271391) c6( ) c7(yL, T, bz); #c1(LHX6) c2(XP_011516824) c3(LIM/homeobox protein Lhx6 isoform X2) c4(310263, 310264) c5( ) c6( ) c7(yL, T, bz); #c1(LHX9) c2(NP_001014434) c3(LIM/homeobox protein Lhx9 isoform 2) c4(25872) c5(25873, 25874, 25875, 25876, 25877, 25878, 25879) c6( ) c7(eA, as); #c1(LHX9) c2(NP_064589) c3(LIM/homeobox protein Lhx9 isoform 1) c4(203141) c5(203142, 203143, 203144, 203145, 203146, 203147) c6( ) c7(eA, as); #c1(LIAS) c2(NP_006850) c3(lipoyl synthase, mitochondrial isoform 1 precursor) c4(168616) c5(168617, 168618, 168619, 168620, 168621, 168622) c6( ) c7(bL, ajU, ha, aj, abj, dL, K, bZp, MC, bZo, eL, UZ, J, hq); #c1(LIAS) c2(NP_919433) c3(lipoyl synthase, mitochondrial isoform 2 precursor) c4(258628) c5(258629, 258630, 258631, 258632, 258633, 258634) c6( ) c7(bL, ajU, ha, aj, abj, dL, K, bZp, MC, bZo, eL, UZ, J, hq); #c1(LIF) c2(XP_011528474) c3 (leukemia inhibitory factor isoform XI) c4(323582) c5(323583, 323584, 323585, 323586, 323587, 323588, 323589) c6( ) c7(bM, T, ya, ea, ci, Qp, Hr, RL, amR, bv, aE, il, M, JT, Ou, K, aVT, azh, Ur, aW, eF, cm, bw, iA, iT, vA, m, dD, lT, aj, fr, PJ, cd, ai, Vs, kH, ey, dx, Lg, mR, aq, op, dB, eD, hR, eo, B, bL, bX, V, eA, J, nv, aT, dz, eu, jf, hM, kl, s, vT, Y, tu, en, wA, t, ck, iM, ac, c, bt, gx, aZ, jh, eq, dP, nJ, d, je, ff, eH, tv, gu, bz, bE, y); #c1(LIFR) c2(XP_011512344) c3(leukemia inhibitory factor receptor isoform X1) c4(302997, 302998, 302999) c5(303000, 303001, 303002, 303003, 303004, 303005, 303006, 303007, 303008, 303009, 303010, 303011, 303012, 303013, 303014, 303015, 303016, 303017, 303018, 303019, 303020, 303021, 303022) c6( ) c7(bM, T, lT, bB, ci, ai, cd, q, ad, eW, bv, Vs, aT, h, K, aVT, bZq, jf, kl, aW, r, aq, ck, bE, mx, m, B); #c1(LIG1) c2(NP_001275992) c3(DNA ligase 1 isoform 2) c4(107677) c5(107678, 107679, 107680, 107681, 107682, 107683) c6( ) c7(av, T, aE, dB, oV, iW, Qk, ew, aT, at, hy, dL, fr, m, bz, eA, ip); #c1(LIG1) c2(NP_001275993) c3(DNA ligase 1 isoform 3) c4(107684) c5(107685, 107686, 107687, 107688, 107689, 107690) c6( ) c7(av, T, aE, dB, oV, iW, Qk, ew, aT, at, hy, dL, fr, m, bz, eA, ip); #c1(LIG1) c2(XP_006723278) c3(DNA ligase 1 isoform X2) c4(295072, 295073) c5(295074, 295075, 295076, 295077, 295078, 295079, 295080, 295081, 295082, 295083, 295084, 295085) c6( ) c7(av, T, aE, dB, oV, iW, Qk, ew, al, at, hy, dL, fr, m, bz, eA, ip); #c1(LIG1) c2(XP_006723279) c3(DNA ligase 1 isoform X1) c4(295086) c5(295087, 295088, 295089, 295090, 295091, 295092) c6( ) c7(av, T, aE, dB, oV, iW, Qk, ew, aT, at, hy, dL, fr, m, bz, eA, ip); #c1(LIG3) c2(XP_005258027) c3(DNA ligase 3 isoform X3) c4(275457) c5(275458, 275459, 275460, 275461, 275462, 275463) c6( ) c7(Gl, aC, aE, aW, iW, ht, EQ, r, aT, lG, hy, dL, at, m, bC, it, Gu, Gw, V, eL); #c1(LIG3) c2(XP_006721959) c3(DNA ligase 3 isoform X5) c4(294493) c5(294494, 294495, 294496, 294497, 294498, 294499) c6( ) c7(Gl, aC, aE, aW, iW, ht, EQ, r, aT, lG, hy, dL, at, m, bC, it, Gu, Gw, V, el); #c1(LIG3) c2(XP_011523099) c3(DNA ligase 3 isoform X1) c4( ) c5(317616) c6( ) c7(Gl, aC, aE, aW, iW, ht, EQ, r, aT, lG, hy, dL, at, m, bC, it, Gu, Gw, V, el); #c1(LIG4) c2(XP_005254115) c3(DNA ligase 4 isoform X1) c4(272967) c5(272968, 272969, 272970, 272971, 272972, 272973, 272974, 272975, 272976, 272977, 272978, 272979, 272980, 272981, 272982) c6( ) c7(bM, T, hl, aC, dA, ajm, Zl, id, if, WL, al, iW, jh, fd, bB, aj, bS, hy, Do, V, q, J, ad, eA, ip, dY, aE, Db, bTr, Qk, gd, aT, dL, K, CB, jf, qw, d, wr, s, aW, Y, oV, ff, CT, r, CF, Kf, en, g, bZs, t, QK, bz, iK, dr, ck, gk, c, nB, dB, bE, eM, bZr, cj, oX, m, k, fD, hh, y, bt, B); #c1(LILRA1) c2(NP_006854) c3(leukocyte immunoglobulin-like receptor subfamily A member 1 isoform 1 precursor) c4(168664) c5( ) c6( ) c7(y, dy, jx, aYt, eZ); #c1(LILRA1) c2(XP_011545378) c3(leukocyte immunoglobulin-like receptor subfamily A member 1 isoform X1) c4( ) c5(344132, 344133, 344134, 344135, 344136, 344137) c6( ) c7(y, dy, jx, aYt, eZ); #c1(LILRA2) c2(NP_006857) c3(leukocyte immunoglobulin-like receptor subfamily A member 2 isoform b precursor) c4(168665) c5(168666, 168667, 168668, 168669, 168670, 168671) c6( ) c7(jx, eZ, Kw, y, hv, aYt); #c1(LILRA2) c2(XP_011545392) c3(leukocyte immunoglobulin-like receptor subfamily A member 2 isoform X2) c4(344138, 344139) c5(344140, 344141, 344142, 344143, 344144, 344145, 344146, 344147, 344148, 344149, 344150, 344151, 344152, 344153, 344154, 344155) c6( ) c7(jx, eZ, Kw, y, hv, aYt); #c1(LILRA3) c2(NP_001166125) c3(leukocyte immunoglobulin-like receptor subfamily A member 3 isoform 2 precursor) c4(68248) c5(68249, 68250, 68251, 68252, 68253, 68254) c6( ) c7(y, V, eo, jx, fN); #c1(LILRA3) c2(XP_011545807) c3(leukocyte immunoglobulin-like receptor subfamily A member 3 isoform X8) c4( ) c5(344663) c6( ) c7(y, V, eo, jx, fN); #c1(LILRA3) c2(XP_011545809) c3(leukocyte immunoglobulin-like receptor subfamily A member 3 isoform X10) c4(344664, 344665) c5(344666, 344667, 344668, 344669, 344670, 344671, 344672, 344673, 344674, 344675, 344676, 344677, 344678, 344679, 344680) c6( ) c7(y, V, eo, jx, fN); #c1(LILRA4) c2(NP_036408) c3(leukocyte immunoglobulin-like receptor subfamily A member 4 precursor) c4(173636) c5(173637, 173638, 173639, 173640, 173641, 173642) c6( ) c7(vi, cj); #c1(LILRA5) c2(NP_067073) c3(leukocyte immunoglobulin-like receptor subfamily A member 5 isoform 1 precursor) c4(208818) c5(208819, 208820, 208821, 208822, 208823, 208824, 208825, 208826) c6(208827, 208828) c7(BU, jx, xR); #c1(LILRA5) c2(NP_870994) c3(leukocyte immunoglobulin-like receptor subfamily A member 5 isoform 3) c4(255913) c5(255914, 255915, 255916, 255917, 255918, 255919, 255920, 255921) c6(255922, 255923) c7(BU, jx, xR); #c1(LILRA5) c2(NP_871714) c3(leukocyte immunoglobulin-like receptor subfamily A member 5 isoform 2) c4(255954) c5(255955, 255956, 255957, 255958, 255959, 255960, 255961, 255962) c6(255963, 255964) c7(BU, jx, xR); #c1(LILRA5) c2(NP_871715) c3(leukocyte immunoglobulin-like receptor subfamily A member 5 isoform 4) c4(255965) c5(255966, 255967, 255968, 255969, 255970, 255971, 255972, 255973) c6(255974, 255975) c7(BU, jx, xR); #c1(LILRB1) c2(NP_001075107) c3(leukocyte immunoglobulin-like receptor subfamily B member 1 isoform 3 precursor) c4(37237) c5( ) c6( ) c7(bL, T, FR, aD, if, aA, bB, aj, v, ci, bX, qN, bWN, q, J, ad, eA, aE, Dq, ds, aT, h, ae, d, aW, Ls, eZ, r, fe, aF, t, bz, Bv, BU, eU, eM, bE, gX, aSu, m, fl, y, bt, B); #c1(LILRB1) c2(NP_001075108) c3 (leukocyte immunoglobulin-like receptor subfamily B member 1 isoform 4 precursor) c4(37238) c5( ) c6( ) c7(bL, T, fR, aD, if, aA, bB, aj, v, ci, bX, qN, bWN, q, J, ad, eA, aE, Dq, ds, aT, h, ae, d, aW, Ls, eZ, r, fe, aF, t, bz, Bv, BU, eU, eM, bE, gX, aSu, m, fl, y, bt, B); #c1(LILRB1) c2(NP_001265327) c3(leukocyte immunoglobulin-like receptor subfamily B member 1 isoform 5 precursor) c4(98274) c5(98275, 98276, 98277, 98278, 98279, 98280) c6( ) c7(bL, T, FR, aD, if, aA, bB, aj, v, ci, bX, qN, bWN, q, J, ad, eA, aE, Dq, ds, aT, h, ae, d, aW, Ls, eZ, r, fe, aF, t, bz, Bv, BU, eU, eM, bE, gX, aSu, m, fl, y, bt, B); #c1(LILRB1) c2(NP_006660) c3(leukocyte immunoglobulin-like receptor subfamily B member 1 isoform 1 precursor) c4(166986) c5(166987, 166988, 166989, 166990, 166991, 166992, 166993, 166994, 166995, 166996, 166997, 166998) c6( ) c7(bL, T, FR, aD, if, aA, bB, aj, v, ci, bX, qN, bWN, q, J, ad, eA, aE, Dq, ds, aT, h, ae, d, aW, Ls, eZ, r, fe, aF, t, bz, Bv, BU, eU, eM, bE, gX, aSu, m, fl, y, bt, B); #c1(LILRB1) c2(XP_011546945) c3(leukocyte immunoglobulin-like receptor subfamily B member 1 isoform X9) c4(345772) c5(345773, 345774, 345775, 345776, 345777, 345778, 345779, 345780, 345781, 345782, 345783, 345784, 345785, 345786, 345787, 345788, 345789, 345790, 345791, 345792) c6( ) c7(bL, T, fR, aD, if, aA, bB, aj, v, ci, bX, qN, bWN, q, J, ad, eA, aE, Dq, ds, aT, h, ae, d, aW, Ls, eZ, r, fe, aF, t, bz, Bv, BU, eU, eM, bE, gX, aSu, m, fl, y, bt, B); #c1(LILRB1) c2(XP_011546950) c3(leukocyte immunoglobulin-like receptor subfamily B member 1 isoform X13) c4(345793) c5(345794, 345795, 345796, 345797, 345798, 345799) c6( ) c7(bL, T, fR, aD, if, aA, bB, aj, v, ci, bX, qN, bWN, q, J, ad, eA, aE, Dq, ds, al, h, ae, d, aW, Ls, eZ, r, fe, aF, t, bz, Bv, BU, eU, eM, bE, gX, aSu, m, fl, y, bt, B); #c1(LILRB2) c2(NP_001265333) c3(leukocyte immunoglobulin-like receptor subfamily B member 2 isoform 3) c4(98281) c5( ) c6( ) c7(T, eM, s, eZ, h, ey, y, bt); #c1(LILRB2) c2(XP_011546934) c3(leukocyte immunoglobulin-like receptor subfamily B member 2 isoform X18) c4(345752) c5(345753, 345754, 345755, 345756, 345757, 345758, 345759, 345760, 345761, 345762, 345763, 345764) c6( ) c7(T, eM, s, eZ, h, ey, y, bt); #c1(LILRB2) c2(XP_011546935) c3(leukocyte immunoglobulin-like receptor subfamily B member 2 isoform X19) c4(345765) c5(345766, 345767, 345768, 345769, 345770, 345771) c6( ) c7(T, eM, s, eZ, h, ey, y, bt); #c1(LILRB3) c2(XP_006726238) c3(leukocyte immunoglobulin-like receptor subfamily B member 3 isoform X10) c4(297126) c5(297127, 297128, 297129, 297130, 297131, 297132) c6( ) c7(LB, aa); #c1(LILRB3) c2(XP_006726239) c3(leukocyte immunoglobulin-like receptor subfamily B member 3 isoform X11) c4(297133) c5(297134, 297135, 297136, 297137, 297138, 297139) c6( ) c7(LB, aa); #c1(LILRB3) c2(XP_011546969) c3(leukocyte immunoglobulin-like receptor subfamily B member 3 isoform X5) c4(345822, 345823) c5( ) c6( ) c7(LB, aa); #c1(LILRB3) c2(XP_011546970) c3(leukocyte immunoglobulin-like receptor subfamily B member 3 isoform X6) c4(345824) c5(345825, 345826, 345827, 345828, 345829, 345830) c6( ) c7(LB, aa); #c1(LILRB3) c2(XP_011546974) c3(leukocyte immunoglobulin-like receptor subfamily B member 3 isoform X12) c4( ) c5(345831, 345832, 345833, 345834, 345835, 345836, 345837, 345838, 345839, 345840, 345841) c6( ) c7(LB, aa); #c1(LILRB4) c2(XP_011546956) c3(leukocyte immunoglobulin-like receptor subfamily B member 4 isoform X2) c4(345800) c5( ) c6( ) c7(T, aC, dA, eZ, fe, fl, bX, iV, mC); #c1(LILRB4) c2(XP_011546957) c3 (leukocyte immunoglobulin-like receptor subfamily B member 4 isoform X3) c4(345801) c5(345802, 345803, 345804, 345805, 345806, 345807, 345808, 345809, 345810, 345811, 345812) c6( ) c7(T, aC, dA, eZ, fe, fl, bX, iV, mC); #c1(LILRB4) c2(XP_011546958) c3(leukocyte immunoglobulin-like receptor subfamily B member 4 isoform X4) c4(345813, 345814) c5( ) c6( ) c7(T, aC, dA, eZ, fe, fL, bX, iV, mC); #c1(LILRB4) c2(XP_011546960) c3(leukocyte immunoglobulin-like receptor subfamily B member 4 isoform X6) c4(345815) c5(345816, 345817, 345818, 345819, 345820, 345821) c6( ) c7(T, aC, dA, eZ, fe, fL, bX, iV, mC); #c1(LILRB5) c2(NP_001074911) c3(leukocyte immunoglobulin-like receptor subfamily B member 5 isoform 1 precursor) c4(37120) c5(37121, 37122, 37123, 37124, 37125, 37126) c6( ) c7(ey); #c1(LILRB5) c2(NP_001074912) c3(leukocyte immunoglobulin-like receptor subfamily B member 5 isoform 3 precursor) c4(37127) c5(37128, 37129, 37130, 37131, 37132, 37133) c6( ) c7(By); #c1(LILRB5) c2(NP_001291386) c3(leukocyte immunoglobulin-like receptor subfamily B member 5 isoform 4 precursor) c4(115833) c5(115834, 115835, 115836, 115837, 115838, 115839) c6( ) c7(ey); #c1(LILRB5) c2(NP_006831) c3(leukocyte immunoglobulin-like receptor subfamily B member 5 isoform 2 precursor) c4(168511) c5(168512, 168513, 168514, 168515, 168516, 168517) c6( ) c7(ey); #c1(LIM2) c2(NP_001155220) c3(lens fiber membrane intrinsic protein isoform 2) c4(61535) c5(61536, 61537, 61538, 61539, 61540, 61541) c6( ) c7(ck, bZt); #c1(LIM2) c2(NP_085915) c3(lens fiber membrane intrinsic protein isoform 1) c4(219632) c5(219633, 219634, 219635, 219636, 219637, 219638) c6( ) c7(ck, bZt); #c1(LIMA1) c2(NP_001230704) c3(LIM domain and actin-binding protein 1 isoform 4) c4( ) c5(86607, 86608, 86609, 86610, 86611, 86612, 86613, 86614, 86615, 86616, 86617) c6( ) c7(T, aW, aT, n, K, ae, ad); #c1(LIMA1) c2(NP_057441) c3(LIM domain and actin-binding protein 1 isoform 2) c4(190862, 190863, 190864, 190865, 190866, 190867, 190868) c5(190869, 190870, 190871, 190872, 190873, 190874, 190875, 190876, 190877, 190878, 190879, 190880, 190881, 190882, 190883, 190884, 190885, 190886, 190887, 190888, 190889, 190890, 190891, 190892, 190893, 190894, 190895, 190896, 190897, 190898, 190899) c6( ) c7(T, aW, aT, n, K, ae, ad); #c1(LIMA1) c2(XP_011536757) c3(LIM domain and actin-binding protein 1 isoform X1) c4(334435, 334436) c5(334437, 334438, 334439, 334440, 334441, 334442, 334443, 334444, 334445, 334446, 334447, 334448) c6( ) c7(T, aW, aT, n, K, ae, ad); #c1(LIMCH1) c2(NP_001106188) c3(LIM and calponin homology domains-containing protein 1 isoform b) c4(42347) c5(42348, 42349, 42350, 42351, 42352, 42353) c6( ) c7(lB); #c1(LIMCH1) c2(NP_001106189) c3(LIM and calponin homology domains-containing protein 1 isoform c) c4(42354) c5(42355, 42356, 42357, 42358, 42359, 42360, 42361, 42362, 42363, 42364, 42365, 42366) c6( ) c7(lB); #c1(LIMCH1) c2(NP_001106190) c3(LIM and calponin homology domains-containing protein 1 isoform d) c4(42367, 42368) c5(42369, 42370, 42371, 42372, 42373, 42374) c6( ) c7(lB); #c1(LIMCH1) c2(NP_001106191) c3(LIM and calponin homology domains-containing protein 1 isoform e) c4(42375, 42376) c5(42377, 42378, 42379, 42380, 42381, 42382) c6( ) c7(lB); #c1(LIMCH1) c2(NP_001276051) c3(LIM and calponin homology domains-containing protein 1 isoform f) c4(107836) c5(107837, 107838, 107839, 107840, 107841, 107842) c6( ) c7(lB); #c1(LIMCH1) c2(NP_055803) c3(LIM and calponin homology domains-containing protein 1 isoform a) c4(183591) c5(183592, 183593, 183594, 183595, 183596, 183597) c6( ) c7(lB); #c1(LIMCH1) c2(XP_005248117) c3(LIM and calponin homology domains-containing protein 1 isoform X7) c4( ) c5(268241) c6( ) c7(lB); #c1(LIMCH1) c2(XP_005248124) c3(LIM and calponin homology domains-containing protein 1 isoform X14) c4(268242, 268243) c5(268244, 268245, 268246, 268247, 268248, 268249, 268250, 268251, 268252, 268253, 268254) c6( ) c7(lB); #c1(LIMCH1) c2(XP_011511960) c3(LIM and calponin homology domains-containing protein 1 isoform X24) c4( ) c5(302606, 302607, 302608, 302609, 302610, 302611) c6( ) c7(lB); #c1(LIMD1) c2(NP_055055) c3(LIM domain-containing protein 1) c4(178630) c5(178631, 178632, 178633, 178634, 178635, 178636, 178637) c6( ) c7(av, T, ff, aT, hy, en, ik, bz); #c1(LIMD2) c2(NP_085053) c3(LIM domain-containing protein 2) c4(219221) c5(219222, 219223, 219224, 219225, 219226, 219227) c6( ) c7(aW, n, bE, bB, aT); #c1(LIMK1) c2(NP_001191355) c3(LIM domain kinase 1 isoform 2) c4(81473) c5(81474, 81475, 81476, 81477, 81478, 81479) c6( ) c7(ID, T, ci, ad, BY, Qk, aT, h, hP, lQ, ae, eu, aW, eH, eF, ek, alC, ac, eM, ei, xg, us); #c1(LIMK1) c2(NP_002305) c3(LIM domain kinase 1 isoform 1) c4(127441) c5(127442, 127443, 127444, 127445, 127446, 127447, 127448, 127449, 127450, 127451, 127452, 127453, 127454) c6( ) c7(ID, T, ci, ad, BY, Qk, aT, h, hP, lQ, ae, eu, aW, eH, eF, eL, alC, ac, eM, ei, xg, us); #c1(LIMK2) c2(NP_001026971) c3(LIM domain kinase 2 isoform 1) c4(30005) c5(30006, 30007, 30008, 30009, 30010, 30011) c6( ) c7(T, gU, eR, ND, r, vH, cm, ji, K, aY, q); #c1(LIMK2) c2(NP_005560) c3(LIM domain kinase 2 isoform 2a) c4(157500) c5(157501, 157502, 157503, 157504, 157505, 157506, 157507) c6( ) c7(T, gU, eR, ND, r, vH, cm, ji, K, aY, q); #c1(LIMK2) c2(NP_057952) c3(LIM domain kinase 2 isoform 2b) c4(192614, 192615, 192616) c5(192617, 192618, 192619, 192620, 192621, 192622, 192623, 192624, 192625, 192626, 192627, 192628, 192629, 192630, 192631, 192632, 192633, 192634) c6( ) c7(T, gU, eR, ND, r, vH, cm, ji, K, aY, q); #c1(LIMS1) c2(NP_001180411) c3(LIM and senescent cell antigen-like-containing domain protein 1 isoform c) c4(73972) c5(73973, 73974, 73975, 73976, 73977, 73978, 73979) c6( ) c7(T, ff, dy, Hb, en, lu, bt, eA); #c1(LIMS1) c2(NP_001180412) c3(LIM and senescent cell antigen-like-containing domain protein 1 isoform d) c4(73980) c5(73981, 73982, 73983, 73984, 73985, 73986) c6( ) c7(T, ff, dy, Hb, en, lu, bt, eA); #c1(LIMS1) c2(NP_001180413) c3(LIM and senescent cell antigen-like-containing domain protein 1 isoform e) c4(73987, 73988) c5(73989, 73990, 73991, 73992, 73993, 73994, 73995) c6( ) c7(T, ff, dy, Hb, en, lu, bt, eA); #c1(LIMS1) c2(NP_001180414) c3(LIM and senescent cell antigen-like-containing domain protein 1 isoform a) c4(73996, 73997) c5(73998, 73999, 74000, 74001, 74002, 74003, 74004) c6( ) c7(T, ff, dy, Hb, en, lu, bt, eA); #c1(LIMS1) c2(NP_001180417) c3(LIM and senescent cell antigen-like-containing domain protein 1 isoform b) c4( ) c5(74012, 74013, 74014, 74015, 74016, 74017, 74018, 74019, 74020, 74021, 74022, 74023, 74024, 74025, 74026, 74027, 74028, 74029, 74030) c6( ) c7(T, ff, dy, Hb, en, lu, bt, eA); #c1(LIMS2) c2(NP_001129509) c3(LIM and senescent cell antigen-like-containing domain protein 2 isoform 1) c4(51771) c5(51772, 51773, 51774, 51775, 51776, 51777, 51778, 51779, 51780, 51781, 51782, 51783, 51784, 51785) c6( ) c7(aA); #c1(LIMS2) c2(NP_001154875) c3(LIM and senescent cell antigen-like-containing domain protein 2 isoform 3) c4(61185) c5(61186, 61187, 61188, 61189, 61190, 61191, 61192) c6( ) c7(aA); #c1(LIMS2) c2(NP_001154876) c3(LIM and senescent cell antigen-like-containing domain protein 2 isoform 5) c4( ) c5(61193, 61194, 61195, 61196, 61197, 61198, 61199, 61200, 61201, 61202, 61203, 61204, 61205, 61206) c6( ) c7(aA); #c1(LIMS2) c2(NP_001243471) c3(LIM and senescent cell antigen-like-containing domain protein 2 isoform 4) c4(90901) c5(90902, 90903, 90904, 90905, 90906, 90907, 90908, 90909, 90910, 90911, 90912, 90913, 90914, 90915, 90916, 90917, 90918) c6( ) c7(aA); #c1(LIMS2) c2(NP_060450) c3(LIM and senescent cell antigen-like-containing domain protein 2 isoform 2) c4(195993, 195994) c5(195995, 195996, 195997, 195998, 195999, 196000) c6( ) c7(aA); #c1(LIMS2) c2(XP_006712690) c3(LIM and senescent cell antigen-like-containing domain protein 2 isoform X4) c4(286798) c5(286799, 286800, 286801, 286802, 286803, 286804, 286805, 286806, 286807, 286808, 286809) c6( ) c7(aA); #c1(LIMS2) c2(XP_011509755) c3(LIM and senescent cell antigen-like-containing domain protein 2 isoform X2) c4(300312) c5(300313, 300314, 300315, 300316, 300317) c6( ) c7(aA); #c1(LIN28B) c2(NP_001004317) c3(protein lin-28 homolog B) c4(20559) c5(20560, 20561, 20562, 20563, 20564, 20565) c6( ) c7(T, Zl, bB, bBX, n, aj, v, q, er, J, Vs, aT, K, jf, aW, ff, r, aq, en, t, cm, eM, bE, m, fD, xb, B); #c1(LIN52) c2(NP_001019845) c3(protein lin-52 homolog) c4(27965) c5(27966, 27967, 27968, 27969, 27970, 27971) c6( ) c7(j); #c1(LIN52) c2(XP_011535622) c3(protein lin-52 homolog isoform X1) c4( ) c5(332882, 332883, 332884, 332885, 332886, 332887, 332888) c6( ) c7(j); #c1(LIN52) c2(XP_011535623) c3(protein lin-52 homolog isoform X2) c4(332889) c5(332890, 332891, 332892, 332893, 332894, 332895) c6( ) c7(j); #c1(LIN7A) c2(NP_004655) c3(protein lin-7 homolog A) c4(148548) c5(148549, 148550, 148551, 148552, 148553, 148554) c6( ) c7(zQ, afy, ci, nA, zP, aqD); #c1(LIN7B) c2(NP_071448) c3(protein lin-7 homolog B) c4(211732) c5(211733, 211734, 211735, 211736, 211737, 211738) c6( ) c7(rz); #c1(LIN7C) c2(NP_060832) c3(protein lin-7 homolog C) c4(198038) c5(198039, 198040, 198041, 198042, 198043, 198044) c6( ) c7(bL, en, v, ff, cA); #c1(LIN9) c2(NP_001257338) c3(protein lin-9 homolog isoform 2) c4(95223) c5(95224, 95225, 95226, 95227, 95228, 95229) c6( ) c7(bP, aW, atC, aT, gZ, B); #c1(LIN9) c2(NP_775106) c3(protein lin-9 homolog isoform 1) c4(249536) c5(249537, 249538, 249539, 249540, 249541, 249542) c6( ) c7(bP, aW, atC, aT, gZ, B); #c1(LIN9) c2(XP_005273159) c3(protein lin-9 homolog isoform X3) c4(284305) c5(284306, 284307, 284308, 284309, 284310, 284311) c6( ) c7(bP, aW, atC, aT, gZ, B); #c1(LIN9) c2(XP_011542475) c3(protein lin-9 homolog isoform X4) c4(340966) c5(340967, 340968, 340969, 340970, 340971, 340972) c6( ) c7(bP, aW, atC, aT, gZ, B); #c1(LINGO1) c2(NP_001288126) c3(leucine-rich repeat and immunoglobulin-like domain-containing nogo receptor-interacting protein 1 isoform b precursor) c4(113568) c5(113569, 113570, 113571, 113572, 113573, 113574, 113575, 113576, 113577, 113578) c6( ) c7(dw, V, Gt, wf, an); #c1(LINGO1) c2(NP_116197) c3(leucine-rich repeat and immunoglobulin-like domain-containing nogo receptor-interacting protein 1 isoform a precursor) c4(225706, 225707) c5(225708, 225709, 225710, 225711, 225712, 225713, 225714, 225715) c6( ) c7(dw, V, Gt, wf, an); #c1(LINGO2) c2(XP_011516032) c3(leucine-rich repeat and immunoglobulin-like domain-containing nogo receptor-interacting protein 2 isoform X1) c4(309227) c5(309228, 309229, 309230, 309231, 309232, 309233, 309234, 309235, 309236, 309237) c6( ) c7(Gt, ac, aw, EQ, aq, v, an, fq, xa); #c1(LINGO4) c2(NP_001004432) c3(leucine-rich repeat and immunoglobulin-like domain-containing nogo receptor-interacting protein 4 precursor) c4(20683) c5(20684, 20685, 20686, 20687, 20688, 20689) c6( ) c7(Gt, byQ); #c1(LINS) c2(NP_001035706) c3 (protein Lines homolog) c4(32975) c5( ) c6( ) c7(QK, bZu, nA); #c1(LINS) c2(XP_005254998) c3(protein Lines homolog isoform X1) c4(273483) c5(273484, 273485, 273486, 273487, 273488, 273489) c6( ) c7(QK, bZu, nA); #c1(LINS) c2(XP_005255000) c3(protein Lines homolog isoform X2) c4(273490, 273491, 273492, 273493) c5(273494, 273495, 273496, 273497, 273498, 273499, 273500, 273501, 273502, 273503, 273504, 273505, 273506, 273507, 273508, 273509, 273510, 273511, 273512, 273513, 273514, 273515, 273516, 273517, 273518, 273519, 273520, 273521, 273522, 273523) c6( ) c7(QK, bZu, nA); #c1(LIPA) c2(NP_000226) c3(lysosomal acid lipase/cholesteryl ester hydrolase isoform 1 precursor) c4(4816, 4817, 4818, 4819, 4820, 4821, 4822, 4823) c5(4824, 4825, 4826, 4827, 4828, 4829, 4830, 4831, 4832, 4833, 4834, 4835, 4836, 4837, 4838, 4839, 4840, 4841, 4842, 4843, 4844, 4845, 4846, 4847, 4848, 4849, 4850, 4851, 4852, 4853, 4854, 4855, 4856, 4857, 4858, 4859, 4860, 4861, 4862, 4863, 4864, 4865, 4866, 4867, 4868, 4869, 4870, 4871, 4872, 4873, 4874, 4875, 4876, 4877, 4878, 4879, 4880, 4881, 4882, 4883, 4884, 4885, 4886, 4887, 4888, 4889, 4890, 4891, 4892, 4893, 4894, 4895, 4896, 4897, 4898, 4899, 4900, 4901, 4902, 4903, 4904, 4905, 4906, 4907, 4908, 4909, 4910, 4911, 4912, 4913, 4914, 4915, 4916, 4917, 4918, 4919, 4920, 4921, 4922, 4923, 4924, 4925, 4926, 4927, 4928, 4929, 4930, 4931, 4932, 4933, 4934, 4935, 4936, 4937, 4938, 4939, 4940, 4941, 4942, 4943, 4944, 4945, 4946, 4947, 4948, 4949, 4950, 4951, 4952, 4953, 4954, 4955, 4956, 4957, 4958, 4959, 4960, 4961, 4962, 4963, 4964, 4965, 4966, 4967, 4968, 4969, 4970, 4971, 4972, 4973, 4974, 4975, 4976, 4977, 4978, 4979, 4980, 4981, 4982, 4983, 4984, 4985, 4986, 4987, 4988, 4989, 4990, 4991, 4992, 4993, 4994, 4995, 4996, 4997, 4998, 4999, 5000, 5001, 5002, 5003, 5004, 5005, 5006, 5007, 5008, 5009, 5010, 5011, 5012, 5013, 5014, 5015, 5016, 5017, 5018, 5019, 5020, 5021, 5022, 5023, 5024, 5025, 5026, 5027, 5028, 5029, 5030, 5031, 5032, 5033, 5034, 5035, 5036, 5037, 5038, 5039, 5040, 5041, 5042, 5043, 5044, 5045, 5046, 5047, 5048, 5049, 5050, 5051, 5052, 5053, 5054, 5055, 5056, 5057, 5058, 5059, 5060, 5061, 5062, 5063, 5064, 5065, 5066, 5067, 5068, 5069, 5070, 5071, 5072, 5073, 5074, 5075, 5076) c6(5077, 5078, 5079, 5080, 5081, 5082, 5083, 5084, 5085, 5086, 5087, 5088, 5089, 5090, 5091, 5092, 5093, 5094, 5095, 5096, 5097) c7(dD, ea, e, aw, cR, v, j, eq, hX, x, h, ey, de, af, bZv, cH, ff, g, en, ej, nT, Lq, k, aEN, aKB, ek); #c1(LIPA) c2(NP_001275908) c3(lysosomal acid lipase/cholesteryl ester hydrolase isoform 2) c4( ) c5(107361, 107362, 107363, 107364, 107365, 107366, 107367) c6(107368) c7(dD, ea, e, aw, cR, v, j, eq, hX, x, h, ey, dQ, af, bZv, cH, ff, g, en, ej, nT, Lq, k, aEN, aKB, ek); #c1(LIPC) c2(XP_005254429) c3(hepatic triacylglycerol lipase isoform X1) c4(273208) c5(273209, 273210, 273211, 273212, 273213, 273214, 273215, 273216, 273217, 273218) c6( ) c7(bL, sY, dD, eR, Zz, bZw, ea, iW, oe, e, aw, cz, vr, Zv, aj, j, ah, C, v, eq, q, J, awq, TB, Zu, dY, ec, YX, vw, xV, cy, dL, jy, h, bq, dQ, db, af, A, nU, cH, cW, fc, r, bZx, Au, cM, uC, Uk, ek, bJ, kT, W, c, ac, cO, alu, Fi, ZD, eM, gZ, at, mf, cS, aS, dy, ei, GF, gT); #c1(LIPE) c2(NP_005348) c3(hormone-sensitive lipase) c4(155289) c5(155290, 155291, 155292, 155293, 155294, 155295) c6( ) c7(bL, T, dD, gU, aZ, ea, dG, j, v, eq, q, td, QJ, vH, jy, qM, kl, ND, r, aq, uC, GR, bJ, Fi, hr, gZ, aoJ, ei, gT, B, ao); #c1(LIPE) c2(XP_005258998) c3(hormone-sensitive lipase isoform X4) c4( ) c5(275915) c6( ) c7(bL, T, dD, gU, aZ, ea, dG, j, v, eq, q, td, AJ, vH, jy, qM, kl, ND, r, aq, uC, GR, bJ, Fi, hr, gZ, aoJ, ei, gT, B, ao); #c1(LIPF) c2(NP_001185757) c3 (gastric triacylglycerol lipase isoform 3 precursor) c4( ) c5(76214, 76215, 76216, 76217, 76218, 76219, 76220) c6(76221, 76222) c7(ej, aW, aKB, aT); #c1(LIPF) c2(NP_001185758) c3(gastric triacylglycerol lipase isoform 1 precursor) c4(76223, 76224) c5(76225, 76226, 76227, 76228, 76229, 76230, 76231, 76232, 76233) c6(76234, 76235) c7(ej, aW, aKB, aT); #c1(LIPF) c2(NP_001185759) c3(gastric triacylglycerol lipase isoform 4 precursor) c4(76236) c5(76237) c6( ) c7(ej, aW, aKB, aT); #c1(LIPF) c2(XP_011538613) c3(gastric triacylglycerol lipase isoform X1) c4( ) c5(336335, 336336, 336337, 336338, 336339, 336340, 336341) c6(336342, 336343) c7(ej, aW, aKB, aT); #c1(LIPG) c2(NP_006024) c3(endothelial lipase precursor) c4(161246) c5(161247, 161248, 161249, 161250, 161251, 161252) c6( ) c7(P, bL, dD, eR, ea, oe, e, aw, cz, v, j, eq, Zu, ql, h, hm, vT, cH, cW, iP, cO, mg, aS, ei); #c1(LIPH) c2(NP_640341) c3(lipase member H precursor) c4(238465) c5(238466, 238467, 238468, 238469, 238470, 238471) c6( ) c7(bZy, YX, xV, bZx, bZz, bZA); #c1(LIPI) c2(NP_001289927) c3(lipase member 1 isoform 1 precursor) c4(114695) c5(114696, 114697, 114698, 114699, 114700, 114701) c6( ) c7(T, bZB, SV); #c1(LIPI) c2(NP_001289928) c3(lipase member 1 isoform 2) c4(114702, 114703) c5(114704, 114705, 114706, 114707, 114708, 114709, 114710, 114711, (14712, 114713, 114714) c6( ) c7(T, bZB, SV); #c1(LIPI) c2(NP_945347) c3(lipase member 1 isoform 5) c4(260857) c5(260858, 260859, 260860, 260861, 260862, 260863, 260864) c6( ) c7(T, bZB, SV); #c1(LIPN) c2(NP_001095939) c3(lipase member N precursor) c4(40945) c5(40946, 40947, 40948, 40949, 40950, 40951) c6( ) c7(aC); #c1(LIPT1) c2(NP_057013) c3(lipoyltransferase 1, mitochondrial precursor) c4(188464, 188465, 188466, 188467, 188468) c5(188469, 188470, 188471, 188472, 188473, 188474, 188475, 188476, 188477, 188478, 188479, 188480, 188481, 188482, 188483, 188484, 188485, 188486, 188487, 188488, 188489, 188490, 188491, 188492, 188493, 188494, 188495, 188496, 188497, 188498, 188499, 188500, 188501, 188502, 188503, 188504, 188505, 188506, 188507, 188508, 188509, 188510, 188511, 188512, 188513, 188514) c6( ) c7(aju, C); #c1(LIPT2) c2(NP_001138341) c3 (putative lipoyltransferase 2, mitochondrial precursor) c4(56185) c5(56186, 56187, 56188, 56189, 56190, 56191) c6( ) c7(fq); #c1(LITAF) c2(NP_001129945) c3(lipopolysaccharide-induced tumor necrosis factor-alpha factor isoform 2) c4(52321, 52322) c5(52323, 52324, 52325, 52326, 52327, 52328, 52329, 52330, 52331, 52332, 52333) c6( ) c7(tJ, T, fM, id, Pt, xF, agU, EQ, cz, aj, aNG, j, ci, bX, MA, hF, q, zn, w, Mm, ec, aT, h, dL, ey, bU, XZ, eL, A, Fn, aW, r, bZC, yy, pD, eF, t, dr, dR, ck, bF, pH, kB, vL, aeg, NO, at, m); #c1(LITAF) c2(XP_006721046) c3(lipopolysaccharide-induced tumor necrosis factor-alpha factor isoform X1) c4( ) c5(293822, 293823, 293824, 293825, 293826, 293827, 293828, 293829) c6( ) c7(tJ, T, fM, id, Pt, xF, agU, EQ, cz, aj, aNG, j, ci, bX, MA, hF, q, zn, w, Mm, ec, aT, h, dL, ey, bU, XZ, eL, A, Fn, aW, r, bZC, yy, pD, eF, t, dr, dR, ck, bF, pH, kB, vL, aeg, NO, at, m); #c1(LITAF) c2(XP_011521056) c3(lipopolysaccharide-induced tumor necrosis factor-alpha factor isoform X2) c4(316027) c5(316028, 316029, 316030, 316031) c6( ) c7(tJ, T, fM, id, Pt, xF, agU, EQ, cz, aj, aNG, j, ci, bX, MA, hF, q, zn, w, Mm, ec, aT, h, dL, ey, bU, XZ, eL, A, Fn, aW, r, bZC, yy, pD, eF, t, dr, dR, ck, bF, pH, KB, vl, aeg, NO, at, m); #c1(LIX1) c2(NP_694966) c3(protein limb expression 1 homolog) c4(245499) c5(245500, 245501, 245502, 245503, 245504, 245505) c6( ) c7(kf); #c1(LLGL1) c2(NP_004131) c3(lethal(2) giant larvae protein homolog 1) c4(143833) c5(143834, 143835, 143836, 143837, 143838, 143839) c6( ) c7(ck, T, alC, s, Y, XY, hL, aT, r, aFx, bfR, ci, q, oQ, bt, B, hq); #c1(LLGL2) c2(NP_001015002) c3 (lethal(2) giant larvae protein homolog 2 isoform b) c4(26128) c5(26129, 26130, 26131, 26132, 26133, 26134) c6( ) c7(T, ac, aW, M, aT, eu); #c1(LLGL2) c2(NP_001026973) c3(lethal(2) giant larvae protein homolog 2 isoform c) c4(30012) c5(30013, 30014, 30015, 30016, 30017, 30018) c6( ) c7(T, ac, aW, M, al, eu); #c1(LLGL2) c2(NP_004515) c3(lethal(2) giant larvae protein homolog 2 isoform a) c4(147099, 147100) c5(147101, 147102, 147103, 147104, 147105, 147106, 147107, 147108, 147109, 147110, 147111, 147112, 147113, 147114) c6(147115, 147116) c7(T, ac, aW, M, aT, eu); #c1(LMAN1) c2(NP_005561) c3(protein ERGIC-53 precursor) c4(157508) c5(157509, 157510, 157511, 157512, 157513, 157514) c6( ) c7(NU, aUK, fR, e, oY, j, ci, bqA, C, hX, qh, dV, acb, ey, bU, bpU, s, mQ, eZ, fe, aUC, gZ, dX); #c1(LMAN1L) c2(NP_068591) c3(protein ERGIC-53-like precursor) c4(209729, 209730, 209731) c5(209732, 209733, 209734, 209735, 209736, 209737, 209738, 209739, 209740, 209741, 209742, 209743, 209744, 209745, 209746, 209747, 209748, 209749) c6( ) c7(pH); #c1(LMAN2L) c2(NP_001135764) c3(VIP36-like protein isoform 1 precursor) c4(53156) c5(53157, 53158, 53159, 53160, 53161, 53162, 53163, 53164) c6( ) c7(fr, bK); #c1(LMAN2L) c2(NP_110432) c3(VIP36-like protein isoform 2 precursor) c4(220333, 220334) c5(220335, 220336, 220337, 220338, 220339, 220340, 220341, 220342, 220343, 220344, 220345, 220346) c6( ) c7(fr, bk); #c1(LMAN2L) c2(XP_011510250) c3(VIP36-like protein isoform X2) c4(300845, 300846) c5(300847, 300848, 300849, 300850, 300851, 300852, 300853, 300854, 300855, 300856) c6( ) c7(fr, bk); #c1(LMBR1) c2(NP_071903) c3(limb region 1 protein homolog) c4(212171, 212172, 212173) c5(212174, 212175, 212176, 212177, 212178, 212179, 212180, 212181, 212182, 212183, 212184, 212185, 212186, 212187, 212188, 212189, 212190, 212191) c6( ) c7(aow, are, bZG, zG, bDk, bZE, bZD, zU, bZF, atb); #c1(LMBR1) c2(XP_005249612) c3(limb region 1 protein homolog isoform X1) c4(269729, 269730, 269731) c5(269732, 269733, 269734, 269735, 269736, 269737, 269738, 269739, 269740, 269741, 269742, 269743, 269744, 269745, 269746, 269747, 269748, 269749) c6( ) c7(aow, are, bZG, zG, bDk, bZE, bZD, zU, bZF, atb); #c1(LMBR1) c2(XP_005249615) c3(limb region 1 protein homolog isoform X4) c4(269750) c5(269751, 269752, 269753, 269754, 269755, 269756) c6( ) c7(aow, are, bZG, zG, bDk, bZE, bZD, zU, bZF, atb); #c1(LMBRD1) c2(NP_060838) c3(probable lysosomal cobalamin transporter) c4(198069, 198070) c5(198071, 198072, 198073, 198074, 198075, 198076, 198077) c6( ) c7(bZH); #c1(LMBRD1) c2(XP_011534243) c3(probable lysosomal cobalamin transporter isoform X1) c4( ) c5(330597, 330598, 330599, 330600, 330601) c6( ) c7(bZH); #c1(LMBRD2) c2(NP_001007528) c3(LMBR1 domain-containing protein 2) c4(22760, 22761) c5(22762, 22763, 22764, 22765, 22766, 22767, 22768, 22769, 22770, 22771, 22772, 22773) c6( ) c7(k); #c1(LMCD1) c2(NP_001265162) c3(LIM and cysteine-rich domains protein 1 isoform 2) c4( ) c5(97886, 97887, 97888, 97889, 97890) c6( ) c7(By, B, K); #c1(LMCD1) c2(NP_001265163) c3(LIM and cysteine-rich domains protein 1 isoform 3) c4(97891) c5(97892, 97893, 97894, 97895, 97896, 97897) c6( ) c7(By, B, K); #c1(LMCD1) c2(NP_001265164) c3(LIM and cysteine-rich domains protein 1 isoform 4) c4(97898) c5(97899, 97900, 97901, 97902, 97903, 97904) c6( ) c7(By, B, K); #c1(LMCD1) c2(NP_055398) c3(LIM and cysteine-rich domains protein 1 isoform 1) c4(180779, 180780) c5(180781, 180782, 180783, 180784, 180785, 180786, 180787, 180788, 180789) c6( ) c7(By, B, K); #c1(LMF1) c2(NP_073610) c3 (lipase maturation factor 1) c4(213124, 213125) c5(213126, 213127, 213128, 213129, 213130, 213131, 213132, 213133, 213134, 213135, 213136, 213137, 213138) c6( ) c7(ar, vG, bZl, bX, GR, Zx); #c1(LMF1) c2(XP_006720989) c3(lipase maturation factor 1 isoform X2) c4(293802, 293803, 293804) c5( ) c6( ) c7(ar, vG, bZl, bX, GR, Zx); #c1(LMF1) c2(XP_011520916) c3 (lipase maturation factor 1 isoform X1) c4(315911, 315912) c5( ) c6( ) c7(ar, vG, bZl, bX, GR, Zx); #c1(LMF1) c2(XP_011520917) c3(lipase maturation factor 1 isoform X5) c4( ) c5(315913, 315914) c6( ) c7(ar, vG, bZl, bX, GR, Zx); #c1(LMF1) c2(XP_011520919) c3 (lipase maturation factor 1 isoform X7) c4( ) c5(315915, 315916, 315917, 315918, 315919, 315920, 315921, 315922, 315923, 315924, 315925, 315926, 315927, 315928, 315929, 315930, 315931) c6( ) c7(ar, vG, bZl, bX, GR, Zx); #c1(LMF1) c2(XP_011520920) c3(lipase maturation factor 1 isoform X8) c4( ) c5(315932, 315933, 315934, 315935, 315936, 315937, 315938, 315939, 315940, 315941, 315942) c6( ) c7(ar, vG, bZl, bX, GR, Zx); #c1(LMLN) c2(NP_001129521) c3(leishmanolysin-like peptidase isoform 1) c4(51825, 51826) c5(51827, 51828, 51829, 51830, 51831, 51832, 51833, 51834, 51835, 51836, 51837, 51838) c6( ) c7(T, qo, Uc, Ol, if, e, aA, it, fr, ci, qN, PJ, asr, q, ad, rc, dP, ft, aE, lg, acu, aCX, jZ, pX, dV, aT, aDt, bOu, NM, ae, VF, d, aW, Y, cr, r, lo, aF, xH, bSZ, Fq, g, kk, ik, bz, op, ck, qe, c, eM, aDs, dc, asq, vA, agl, DC, Nl, uz, ER, eg, bt, Pr, B); #c1(LMLN) c2(NP_149018) c3(leishmanolysin-like peptidase isoform 2) c4(226769) c5(226770, 226771, 226772, 226773, 226774, 226775) c6( ) c7(T, qo, Uc, Ol, if, e, aA, it, fr, ci, qN, PJ, asr, q, ad, rc, dP, ft, aE, lg, acu, aCX, jZ, pX, dV, aT, aDt, bOu, NM, ae, VF, d, aW, Y, cr, r, lo, aF, xH, bSZ, Fq, g, kk, iK, bz, op, ck, qe, c, eM, aDs, dc, asq, vA, agl, DC, Nl, uz, ER, eg, bt, Pr, B); #c1(LMNA) c2(NP_001244303) c3(lamin isoform D) c4(92108) c5(92109, 92110, 92111, 92112, 92113, 92114) c6( ) c7(T, rV, ea, qj, ci, x, rw, Eo, K, bU, bc, bZR, azu, aCg, aW, Az, La, g, cm, GR, ly, wK, mx, aep, U, bel, cx, bZO, k, in, iu, dD, bAB, aWG, n, arl, aj, fr, cd, eW, md, jz, no, cc, rC, Ak, h, jy, bl, Dz, uc, pj, asp, aiB, lb, mo, bxJ, bfW, GG, bP, afc, mg, rx, YH, ZY, bD, Pr, bL, bZJ, fM, eR, mr, wL, fG, rf, cz, cR, aTl, v, j, bX, nS, V, AD, bZP, J, q, QI, MR, nv, nX, aT, kf, adm, aWs, jj, avt, aOV, ae, bZN, af, VC, avg, kl, wr, iU, NE, blV, r, eS, bZM, aHG, ck, bLr, ac, bNJ, mG, km, JL, mf, Gg, aan, bAQ, id, atk, aZ, jh, eq, fu, tO, ad, KL, hX, fU, LL, Db, Dq, QJ, oj, vc, anq, anR, bZL, qt, KS, xs, eZ, bZA, dq, aF, Bu, bz, dr, gZ, Km, aS, Fw, ei, QV, aed, bZK, gq, jr, pC); #c1(LMNA) c2(NP_001269553) c3(lamin isoform E) c4(101908) c5(101909, 101910, 101911, 101912, 101913, 101914, 101915, 101916, 101917, 101918) c6( ) c7(T, rV, ea, qj, ci, x, rw, Eo, K, bU, bc, bZR, azu, aCg, aW, Az, La, g, cm, GR, ly, wK, mx, aep, U, bel, cx, bZO, k, in, iu, dD, bAB, aWG, n, arl, aj, fr, cd, eW, md, jz, no, cc, rC, Ak, h, jy, bl, Dz, uc, pj, asp, aiB, lb, mo, bxJ, bfW, GG, bP, afc, mg, rx, YH, ZY, bD, Pr, bL, bZJ, fM, eR, mr, wl, fG, rf, cz, cR, aTl, v, j, bX, nS, V, AD, bZP, J, q, QI, MR, nv, nX, aT, kf, adm, aWs, jj, avt, aOV, ae, bZN, af, VC, avg, kl, wr, il, NE, blV, r, eS, bZM, aHG, ck, bLr, ac, bNJ, mG, km, JL, mf, Gg, aan, bAQ, id, atk, aZ, jh, eq, fu, tO, ad, KL, hX, fU, LL, Db, Dq, QJ, oj, vc, anq, anR, bZL, qt, KS, xs, eZ, bZQ, dq, aF, Bu, bz, dr, gZ, Km, aS, Fw, ei, QV, aed, bZK, gq, jr, pC); #c1(LMNA) c2(NP_001269554) c3(lamin isoform C) c4(101919) c5(101920, 101921, 101922, 101923, 101924, 101925, 101926, 101927, 101928, 101929, 101930, 101931, 101932, 101933, 101934, 101935, 101936, 101937, 101938, 101939, 101940, 101941, 101942) c6( ) c7(T, rV, ea, qj, ci, x, rw, Eo, K, bU, bc, bZR, azu, aCg, aW, Az, La, g, cm, GR, ly, wK, mx, aep, U, bel, cx, bZO, k, in, iu, dD, bAB, aWG, n, arl, aj, fr, cd, eW, md, jz, no, cc, rC, Ak, h, jy, bl, Dz, uc, pj, asp, aiB, lb, mo, bxJ, bfW, GG, bP, afc, mg, rx, YH, ZY, bD, Pr, bL, bZJ, fM, eR, mr, wL, fG, rf, cz, cR, aTl, v, j, bX, nS, V, AO, bZP, J, q, QI, MR, nv, nX, aT, kf, adm, aWs, jj, avt, aOV, ae, bZN, af, VC, avg, kl, wr, il, NE, blV, r, eS, bZM, aHG, ck, bLr, ac, bNJ, mG, km, JL, mf, Gg, aan, bAQ, id, atk, aZ, jh, eq, fu, tO, ad, kL, hX, fU, LL, Db, Dq, QJ, oj, vc, anq, anR, bZL, qt, KS, xs, eZ, bZA, dq, aF, Bu, bz, dr, gZ, Km, aS, Fw, ei, QV, aed, bZK, gq, jr, pC); #c1(LMNA) c2(NP_001269555) c3(lamin isoform A-delta50) c4(101943, 101944) c5(101945, 101946, 101947, 101948, 101949, 101950, 101951, 101952) c6( ) c7(T, rV, ea, qj, ci, x, rw, Eo, K, bU, bc, bZR, azu, aCg, aW, Az, La, g, cm, GR, ly, wK, mx, aep, U, bel, cX, bZO, k, in, iu, dD, bAB, aWG, n, arl, aj, fr, cd, eW, md, jz, no, cc, rC, Ak, h, jy, bl, Dz, uc, pj, asp, aiB, lb, mo, bxJ, bfW, GG, bP, afc, mg, rx, YH, ZY, bD, Pr, bL, bZJ, fM, eR, mr, wL, fG, rf, cz, cR, aTl, v, j, bX, nS, V, AD, bZP, J, q, QI, MR, nv, nX, aT, kf, adm, aWs, jj, avt, aOV, ae, bZN, af, VC, avg, kl, wr, il, NE, blV, r, eS, bZM, aHG, ck, bLr, ac, bNJ, mG, km, JL, mf, Gg, aan, bAQ, id, atk, aZ, jh, eq, fu, tO, ad, KL, hX, fU, LL, Db, Dq, QJ, oj, vc, anq, anR, bZL, qt, KS, xs, eZ, bZA, dq, aF, Bu, bz, dr, gZ, Km, aS, Fw, ei, QV, aed, bZK, gq, jr, pC); #c1(LMNA) c2(NP_733821) c3(lamin isoform A) c4(247166) c5(247167, 247168, 247169, 247170, 247171, 247172, 247173, 247174, 247175) c6( ) c7(T, rV, ea, qj, ci, x, rw, Eo, K, bU, bc, bZR, azu, aCg, aW, Az, La, g, cm, GR, ly, wK, mx, aep, U, bel, cX, bZO, k, in, iu, dD, bAB, aWG, n, arl, aj, fr, cd, eW, md, jz, no, cc, rC, Ak, h, jy, bl, Dz, uc, pj, asp, aiB, lb, mo, bxJ, bfW, GG, bP, afc, mg, rx, YH, ZY, bD, Pr, bL, bZJ, fM, eR, mr, wL, fG, rf, cz, cR, aTl, v, j, bX, nS, V, AD, bZP, J, q, QI, MR, nv, nX, aT, kf, adm, aWs, jj, avt, aOV, ae, bZN, af, VC, avg, kl, wr, il, NE, blV, r, eS, bZM, aHG, ck, bLr, ac, bNJ, mG, km, JL, mf, Gg, aan, bAQ, id, atk, aZ, jh, eq, fu, to, ad, kL, hX, fU, LL, Db, Dq, QJ, oj, vc, anq, anR, bZL, qt, KS, xs, eZ, bZQ, dq, aF, Bu, bz, dr, gZ, Km, aS, Fw, ei, QV, aed, bZK, gq, jr, pC); #c1(LMNA) c2(NP_733822) c3(lamin isoform A-delta10) c4(247176, 247177) c5(247178, 247179, 247180, 247181, 247182, 247183, 247184) c6( ) c7(T, rV, ea, qj, ci, x, rw, Eo, K, bU, bc, bZR, azu, aCg, aW, Az, La, g, cm, GR, ly, wK, mx, aep, U, bel, cx, bZO, k, in, iu, dD, bAB, aWG, n, arl, aj, fr, cd, eW, md, jz, no, cc, rC, Ak, h, jy, bl, Dz, uc, pj, asp, aiB, lb, mo, bxJ, bfW, GG, bP, afc, mg, rx, YH, ZY, bD, Pr, bL, bZJ, fM, eR, mr, wL, fG, rf, cz, cR, aTl, v, j, bX, nS, V, AD, bZP, J, q, Ql, MR, nv, nX, aT, kf, adm, aWs, jj, avt, aOV, ae, bZN, af, VC, avg, kl, wr, iU, NE, blV, r, eS, bZM, aHG, ck, bLr, ac, bNJ, mG, km, JL, mf, Gg, aan, bAQ, id, atk, aZ, jh, eq, fu, tO, ad, KL, hX, fU, LL, Db, Dq, QJ, oj, vc, anq, anR, bZL, qt, KS, xs, eZ, bZA, dq, aF, Bu, bz, dr, gZ, Km, aS, Fw, ei, QV, aed, bZK, gq, jr, pC); #c1(LMNB1) c2(NP_005564) c3(lamin-B1 isoform 1) c4(157515) c5(157516, 157517, 157518, 157519, 157520, 157521) c6( ) c7(T, dD, ea, HM, eq, mF, V, eW, aE, QJ, rC, ey, K, cb, bZT, aq, iR, c, kB, U, bZS, cS, bee, B); #c1(LMNB2) c2(NP 116126) c3(lamin-B2) c4(225436) c5(225437, 225438, 225439, 225440, 225441, 225442, 225443, 225444, 225445, 225446) c6( ) c7(Kl, KE, LL, lR, vW, xA, eR, aA, vw, An, aF, dy, ah, aSO, Fw, jj, BB, GR); #c1(LMNTD1) c2(NP_001139199) c3(lamin tail domain-containing protein 1 isoform 3) c4(58948) c5(58949, 58950, 58951, 58952, 58953, 58954, 58955) c6( ) c7(AW, fq, cd); #c1(LMNTD1) c2(NP_001243195) c3(lamin tail domain-containing protein 1 isoform 5) c4( ) c5(90094, 90095, 90096, 90097, 90098) c6( ) c7(AW, fq, cd); #c1(LMNTD1) c2(NP_689803) c3(lamin tail domain-containing protein 1 isoform 2) c4(244009) c5(244010, 244011, 244012, 244013, 244014, 244015) c6( ) c7(AW, fq, cd); #c1(LMNTD1) c2(XP_011518880) c3(lamin tail domain-containing protein 1 isoform X2) c4(313219, 313220, 313221) c5( ) c6( ) c7(AW, fq, cd); #c1(LMNTD1) c2(XP_011518881) c3(lamin tail domain-containing protein 1 isoform X3) c4( ) c5(313222, 313223, 313224, 313225, 313226, 313227) c6( ) c7(AW, fq, cd); #c1(LMNTD1) c2(XP_011518882) c3(lamin tail domain-containing protein 1 isoform X1) c4(313228, 313229) c5(313230, 313231, 313232, 313233, 313234, 313235) c6( ) c7(AW, fq, cd); #c1(LMNTD1) c2(XP_011518890) c3(lamin tail domain-containing protein 1 isoform X6) c4( ) c5(313236, 313237, 313238, 313239, 313240, 313241, 313242, 313243, 313244, 313245, 313246, 313247) c6( ) c7(AW, fq, cd); #c1(LMO1) c2(NP_001257357) c3(rhombotin-1 isoform b) c4(95254) c5(95255, 95256, 95257, 95258, 95259, 95260) c6( ) c7(ck, x, T, iM, je, aW, oe, apK, aT, kH, cj, r, aj, cm, k, hF, q, ek); #c1(LMO1) c2(NP_002306) c3(rhombotin-1 isoform a) c4(127455) c5(127456, 127457, 127458, 127459, 127460, 127461, 127462) c6( ) c7(ck, x, T, iM, je, aW, oe, apK, aT, kH, cj, r, aj, cm, k, hF, q, ek); #c1(LMO2) c2(NP_001135787) c3(rhombotin-2 isoform 2) c4( ) c5(53282, 53283, 53284, 53285, 53286) c6( ) c7(T, fM, dA, eX, jh, Hn, hF, pd, ad, x, av, aE, aT, kH, eV, dL, ae, je, qt, aW, jg, cm, LV, ck, iM, bP, iT, at, k, bt); #c1(LMO2) c2(NP_005565) c3(rhombotin-2 isoform 1) c4(157522, 157523) c5(157524, 157525, 157526, 157527, 157528, 157529, 157530, 157531, 157532) c6( ) c7(T, fM, dA, eX, jh, Hn, hF, pd, ad, x, av, aE, aT, kH, eV, dL, ae, je, qt, aW, jg, cm, LV, ck, iM, bP, iT, at, k, bt); #c1(LMO3) c2(NP_001001395) c3(LIM domain only protein 3 isoform 1) c4( ) c5(18254, 18255, 18256, 18257, 18258, 18259, 18260, 18261, 18262, 18263, 18264, 18265, 18266, 18267, 18268, 18269, 18270, 18271, 18272, 18273, 18274, 18275, 18276, 18277, 18278, 18279, 18280, 18281, 18282, 18283, 18284, 18285, 18286, 18287, 18288, 18289, 18290, 18291, 18292, 18293, 18294, 18295, 18296, 18297, 18298, 18299, 18300, 18301, 18302, 18303, 18304, 18305, 18306, 18307, 18308, 18309, 18310, 18311, 18312, 18313, 18314, 18315, 18316, 18317, 18318, 18319, 18320, 18321, 18322, 18323) c6( ) c7(T, aUk, v, Jd, Ea, bZV, VV, ix, Sb, bZU, gl, K, bK, he, bME, aqa, aWm, aog, yL, bds, g, Eg, FX, cm, bz, eM, Qu, amu, Gp, iu); #c1(LMO3) c2(NP_001230540) c3(LIM domain only protein 3 isoform 2) c4(86324) c5(86325, 86326, 86327, 86328, 86329, 86330, 86331, 86332, 86333, 86334, 86335, 86336, 86337, 86338, 86339, 86340, 86341, 86342, 86343, 86344, 86345, 86346, 86347) c6( ) c7(T, aUk, v, Jd, Ea, bZV, VV, iX, Sb, bZU, gl, K, bK, he, bME, aqa, aWm, aog, yL, bds, g, Eg, FX, cm, bz, eM, Qu, amu, Gp, iu); #c1(LMO3) c2(NP_001230541) c3(LIM domain only protein 3 isoform 3) c4(86348, 86349, 86350, 86351, 86352, 86353, 86354, 86355, 86356) c5(86357, 86358, 86359, 86360, 86361, 86362, 86363, 86364) c6( ) c7(T, aUk, v, Jd, Ea, bZV, VV, iX, Sb, bZU, gl, K, bK, he, bME, aqa, aWm, aog, yL, bds, g, Eg, FX, cm, bz, eM, Qu, amu, Gp, iu); #c1(LMO7) c2(NP_005349) c3(LIM domain only protein 7 isoform 1) c4(155296) c5(155297, 155298, 155299, 155300, 155301, 155302, 155303) c6( ) c7(A, T, oe, ad, oj); #c1(LMO7) c2(NP_056667) c3(LIM domain only protein 7 isoform 2) c4(188113) c5(188114, 188115, 188116, 188117, 188118) c6( ) c7(A, T, De, ad, oj); #c1(LMOD1) c2(NP_036266) c3(leiomodin-1) c4(172706, 172707, 172708, 172709) c5(172710, 172711, 172712, 172713, 172714, 172715, 172716, 172717, 172718, 172719, 172720, 172721, 172722, 172723, 172724, 172725, 172726, 172727) c6( ) c7(dP, A, aaG, hr, HD, at, dL, act, an); #c1(LMOD3) c2(NP_001291347) c3(leiomodin-3) c4(115668) c5(115669, 115670, 115671, 115672, 115673, 115674, 115675, 115676, 115677, 115678, 115679, 115680, 115681, 115682) c6( ) c7(Am); #c1(LMTK2) c2(NP_055731) c3(serine/threonine-protein kinase LMTK2 precursor) c4(183167) c5(183168, 183169, 183170, 183171, 183172, 183173) c6( ) c7(g, ae, iu, ad, Ea); #c1(LMTK3) c2(NP_001073903) c3(serine/threonine-protein kinase LMTK3) c4(36419) c5(36420, 36421, 36422, 36423, 36424, 36425) c6( ) c7(aW, aF, aA, aT); #c1(LMX1A) c2(NP_001167540) c3(LIM homeobox transcription factor 1-alpha) c4(69042) c5(69043, 69044, 69045, 69046, 69047, 69048, 69049, 69050, 69051, 69052, 69053, 69054, 69055, 69056) c6( ) c7(bL, if, aA, cz, n, dL, aF, m, Ea, an, qN, t, eL, bA); #c1(LMX1A) c2(XP_011507841) c3(LIM homeobox transcription factor 1-alpha isoform X2) c4( ) c5(297981) c6( ) c7(bL, if, aA, cz, n, dL, aF, m, Ea, an, qN, t, eL, bA); #c1(LMX1B) c2(NP_001167617) c3(LIM homeobox transcription factor 1-beta isoform 3) c4(69414) c5(69415, 69416, 69417, 69418, 69419, 69420) c6( ) c7(T, bO, bZW, pn, er, Na, XW, MS, Nc, an, dQ, he, wt, Cr, dH, byn, ih, wf, bZX, dw, t, fq, bUq, bN, zV, bbE, pZ, dy, m, sH); #c1(LMX1B) c2(NP_001167618) c3(LIM homeobox transcription factor 1-beta isoform 2) c4(69421) c5(69422, 69423, 69424, 69425, 69426, 69427) c6( ) c7(T, bO, bZW, pn, er, Na, XW, MS, Nc, an, dQ, he, wt, Cr, dH, byn, ih, wf, bZX, dw, t, fq, bUq, bN, zV, bbE, pZ, dy, m, sH); #c1(LMX1B) c2(NP_002307) c3(LIM homeobox transcription factor 1-beta isoform 1) c4(127463, 127464) c5(127465, 127466, 127467, 127468, 127469, 127470, 127471, 127472, 127473, 127474, 127475, 127476, 127477) c6( ) c7(T, bO, bZW, pn, er, Na, XW, MS, Nc, an, dQ, he, wt, Cr, dH, byn, ih, wf, bZX, dw, t, fq, bUq, bN, zV, bbE, pZ, dy, m, sH); #c1(LNPEP) c2(NP_005566) c3(leucyl-cystinyl aminopeptidase isoform 1) c4(157533) c5(157534, 157535, 157536, 157537, 157538, 157539, 157540, 157541, 157542, 157543, 157544, 157545) c6( ) c7(bL, bM, T, jv, Ke, wu, e, jx, cz, bB, n, aj, v, fr, lu, zP, q, J, eW, F, ot, di, afn, aT, h, wm, MO, nJ, avR, aW, cr, r, tv, aq, tu, g, cl, t, uC, avT, es, cT, rR, avS, bE, AM, dJ, m, y, B); #c1(LNPEP) c2(NP_787116) c3(leucyl-cystinyl aminopeptidase isoform 2) c4(252127) c5( ) c6( ) c7(bL, bM, T, jv, Ke, wu, e, jx, cz, bB, n, aj, v, fr, lu, zP, q, J, eW, F, ot, di, afn, aT, h, wm, MO, nJ, avR, aW, cr, r, tv, aq, tu, g, cl, t, uC, avT, es, cT, rR, avS, bE, AM, dJ, m, y, B); #c1(LNX1) c2(NP_116011) c3(E3 ubiquitin-protein ligase LNX isoform b precursor) c4(225198) c5(225199, 225200, 225201, 225202, 225203, 225204) c6( ) c7(wQ, aap, d, lR, oe, lf, ab, bZY, Ea, FX, eA); #c1(LNX1) c2(XP_005265843) c3(E3 ubiquitin-protein ligase LNX isoform X1) c4(279741, 279742, 279743, 279744, 279745, 279746) c5(279747, 279748, 279749, 279750, 279751, 279752, 279753, 279754, 279755, 279756, 279757, 279758, 279759, 279760, 279761, 279762, 279763, 279764, 279765, 279766, 279767, 279768, 279769, 279770, 27971, 279772, 279773, 27974, 27975, 279776, 279777, 279778, 279779, 279780, 279781, 279782) c6( ) c7(wQ, aap, d, lR, oe, lf, ab, bZY, Ea, FX, eA); #c1(LNX2) c2(NP_699202) c3(ligand of Numb protein X 2) c4(245898) c5(245899, 245900, 245901, 245902, 245903, 245904) c6( ) c7(aw, q, jh, r); #c1(LOC100288966) c2(NP_001244291) c3(uncharacterized protein LOC100288966) c4(92101) c5(92102, 92103, 92104, 92105, 92106, 92107) c6( ) c7(T, aW, aT, m, bu, t, ae, ad); #c1(LOC101060321) c2(NP_001278391) c3(TBC1 domain family member-like) c4(109611, 109612, 109613, 109614, 109615, 109616) c5(109617, 109618, 109619, 109620, 109621, 109622, 109623, 109624, 109625, 109626, 109627, 109628, 109629, 109630, 109631, 109632, 109633, 109634, 109635, 109636, 109637, 109638, 109639, 109640, 109641, 109642, 109643, 109644, 109645, 109646, 109647, 109648, 109649, 109650, 109651, 109652, 109653, 109654, 109655, 109656, 109657, 109658, 109659, 109660, 109661, 109662, 109663, 109664, 109665, 109666) c6( ) c7(ae, ad); #c1(LOC101060321) c2(XP_006725438) c3(TBC1 domain family member-like isoform X1) c4( ) c5(296806, 296807) c6( ) c7(ae, ad); #c1(LOC102723475) c2(XP_006724009) c3(potassium voltage-gated channel subfamily E member 1 isoform X1) c4( ) c5(295589, 295590, 295591, 295592, 295593, 295594, 295595, 295596, 295597, 295598) c6( ) c7(bUz); #c1(LOC102723996) c2(XP_006723962) c3(ICOS ligand isoform X1) c4( ) c5(295579, 295580) c6( ) c7(Nq, Kd, ck, Nr, OW, Bv, bM, A, Dq, Y, aA, po, wV, aF, iV, aaO, y, bt, eA, Wt); #c1(LOC102723996) c2(XP_006723963) c3(ICOS ligand isoform X4) c4(295581) c5(295582, 295583, 295584, 295585, 295586, 295587, 295588) c6( ) c7(Nq, Kd, ck, Nr, OW, Bv, bM, A, Dq, Y, aA, po, wV, aF, iV, aaO, y, bt, eA, Wt); #c1(LOC102724127) c2(XP_011544335) c3(TP53-target gene 3 protein) c4(343460) c5(343461, 343462, 343463, 343464, 343465, 343466, 343467, 343468, 343469, 343470, 343471, 343472) c6( ) c7(T); #c1(LOC102724560) c2(XP_011544402) c3(cystathionine beta-synthase isoform X2) c4(343473, 343474, 343475, 343476) c5(343477, 343478, 343479, 343480, 343481, 343482, 343483, 343484, 343485, 343486, 343487, 343488, 343489, 343490, 343491, 343492, 343493, 343494, 343495, 343496, 343497, 343498, 343499, 343500, 343501, 343502, 343503, 343504, 343505, 343506, 343507, 343508, 343509, 343510, 343511, 343512) c6( ) c7(ng, T, dD, ea, iW, e, al, fd, aA, sM, cR, aj, v, j, ci, C, nA, bB, aY, eq, q, J, ad, ip, av, M, aT, iY, dL, aBR, K, ux, is, aBQ, ae, dF, af, eL, MX, aW, oV, PX, Zf, fc, r, dN, aq, fe, aF, t, f, bz, dr, bN, eK, ck, bF, mO, rR, lU, ac, lp, bE, ag, aBO, ayS, Nb, gX, fP, uo, wE, wR, aS, m, cP, bt, B, us); #c1(LOC102724770) c2(XP_006724997) c3(protein DGCR6) c4(296508) c5(296509, 296510, 296511, 296512, 296513, 296514) c6( ) c7(RX, agd, bN, hq); #c1(LOC102725016) c2(XP_006725712) c3(serine/threonine-protein phosphatase 2A regulatory subunit B″ subunit beta isoform X1) c4(296963) c5(296964) c6( ) c7(vi); #c1(LOC102725016) c2(XP_011545804) c3(serine/threonine-protein phosphatase ZA regulatory subunit B″ subunit beta isoform X2) c4( ) c5(344662) c6( ) c7(vi); #c1(LOC102725035) c2(XP_006726376) c3(leukocyte immunoglobulin-like receptor subfamily B member 3 isoform X6) c4(297147, 297148, 297149) c5(297150, 297151, 297152, 297153, 297154, 297155, 297156, 297157, 297158, 297159, 297160, 297161, 297162, 297163, 297164, 297165, 297166, 297167) c6( ) c7(LB); #c1(LOC102725035) c2(XP_006726377) c3(leukocyte immunoglobulin-like receptor subfamily B member 3 isoform X7) c4(297168, 297169) c5(297170, 297171, 297172, 297173, 297174, 297175, 297176, 297177, 297178, 297179, 297180, 297181, 297182, 297183, 297184, 297185, 297186, 297187, 297188, 297189, 297190, 297191, 297192) c6( ) c7(LB); #c1(LOC102725035) c2(XP_011546877) c3(leukocyte immunoglobulin-like receptor subfamily B member 3 isoform X2) c4(345731, 345732) c5(345733, 345734, 345735, 345736, 345737, 345738) c6( ) c7(LB); #c1(LOC400499) c2(XP_006721060) c3(putative uncharacterized protein LOC400499 isoform X2) c4(293831) c5(293832, 293833, 293834, 293835, 293836, 293837, 293838) c6( ) c7(j); #c1(LOC400927-CSNKIE) c2(NP_001276841) c3(casein kinase 1 isoform epsilon) c4(108465, 108466, 108467, 108468) c5(108469, 108470, 108471, 108472, 108473, 108474, 108475, 108476, 108477, 108478, 108479, 108480, 108481, 108482, 108483, 108484, 108485, 108486, 108487, 108488, 108489, 108490, 108491, 108492, 108493, 108494, 108495, 108496, 108497, 108498, 108499, 108500, 108501, 108502, 108503, 108504, 108505, 108506, 108507, 108508) c6( ) c7(T, aWm, aW, alR, aT, Nt, gl, aqm, bK, ae, ad); #c1(LOC645177) c2(XP_011519208) c3(lymphoid-restricted membrane protein-like) c4( ) c5(313790) c6( ) c7(aw); #c1(LONP1) c2(NP_001263408) c3(Ion protease homolog, mitochondrial isoform 2) c4( ) c5(97277) c6( ) c7(ID, T, am, fM, eX, jh, aA, fd, aj, Gn, ci, sl, cd, J, ad, cc, aE, bQV, rA, kH, aT, cy, h, eV, K, ae, N, eu, ly, xX, d, bZZ, aW, ff, hl, kp, aF, uF, en, hf, ik, bz, GR, LS, lx, Cv, ck, eM, bE, cj, bt, it, jr); #c1(LONP1) c2(NP_001263409) c3(Ion protease homolog, mitochondrial isoform 3) c4(97278) c5(97279, 97280, 97281, 97282, 97283) c6( ) c7(ID, T, am, fM, eX, jh, aA, fd, aj, Gn, ci, sl, cd, J, ad, cc, aE, bQV, rA, kH, aT, cy, h, eV, K, ae, N, eu, ly, xX, d, bZZ, aW, ff, hl, kp, aF, uF, en, hf, ik, bz, GR, LS, lx, Cv, ck, eM, bE, cj, bt, it, jr); #c1(LONP1) c2(NP_004784) c3(Ion protease homolog, mitochondrial isoform 1 precursor) c4(149615, 149616) c5(149617, 149618, 149619, 149620, 149621, 149622, 149623, 149624, 149625, 149626, 149627, 149628) c6( ) c7(ID, T, am, fM, eX, jh, aA, fd, aj, Gn, ci, sl, cd, J, ad, cc, aE, bQV, rA, kH, aT, cy, h, eV, K, ae, N, eu, ly, xX, d, bZZ, aW, ff, hl, kp, aF, uF, en, hf, iK, bz, GR, LS, lx, Cv, ck, eM, bE, cj, bt, it, jr); #c1(LONRF1) c2(NP_689484) c3(LON peptidase N-terminal domain and RING finger protein 1) c4(242841) c5(242842, 242843, 242844, 242845, 242846, 242847) c6( ) c7(oe, q); #c1(LOR) c2(NP_000418) c3(loricrin) c4(8778, 8779) c5(8780, 8781) c6( ) c7(T, bCq, iM, lU, bnY, je, bVu, arE, hk, ff, cr, by, fn, lQ, Xk, en, eE, bc); #c1(LOR) c2(XP_011507843) c3(loricrin isoform X1) c4( ) c5(297982, 297983, 297984, 297985, 297986, 297987, 297988, 297989, 297990, 297991, 297992) c6( ) c7(T, bCq, iM, lU, bnY, je, bVu, arE, hk, ff, cr, by, fn, lQ, Xk, en, eE, bc); #c1(LOX) c2(NP_002308) c3(protein-lysine 6-oxidase isoform 1 preproprotein) c4(127478, 127479, 127480) c5(127481, 127482, 127483, 127484, 127485, 127486, 127487, 127488, 127489, 127490, 127491, 127492, 127493, 127494, 127495, 127496, 127497, 127498) c6( ) c7(T, aC, lP, ea, e, fd, bB, fo, ci, cab, aE, ws, K, eL, bc, cp, iE, aW, eF, iP, eY, xd, eM, U, uo, uJ, m, Vj, dD, cd, aOu, ep, h, dL, vx, dx, pl, MX, zq, aq, vD, bm, pg, alo, it, B, fl, fG, jx, aA, cR, tc, cz, j, Eh, q, eA, aT, ae, bA, oV, Y, la, r, iy, en, t, amF, c, dh, aZ, eq, ad, hX, av, le, ab, fn, lQ, aln, alk, d, lU, cH, qt, ff, eH, dq, aF, si, iR, bz, bF, caa, akQ, Uj, bE, gZ, aS, X, us); #c1(LOXHD1) c2(NP_001138944) c3(lipoxygenase homology domain-containing protein 1 isoform 2) c4(58447) c5(58448, 58449, 58450, 58451, 58452, 58453, 58454, 58455, 58456, 58457, 58458) c6( ) c7(cac, Lu, bCJ); #c1(LOXHD1) c2(NP_001138945) c3(lipoxygenase homology domain-containing protein 1 isoform 3) c4( ) c5(58459, 58460, 58461, 58462, 58463) c6( ) c7(cac, Lu, bCJ); #c1(LOXHD1) c2(NP_001166600) c3(lipoxygenase homology domain-containing protein 1 isoform 4) c4( ) c5(68569, 68570, 68571, 68572, 68573) c6( ) c7(cac, Lu, bCJ); #c1(LOXHD1) c2(NP_653213) c3(lipoxygenase homology domain-containing protein 1 isoform 1) c4(239094, 239095, 239096) c5(239097, 239098, 239099, 239100, 239101, 239102, 239103, 239104, 239105, 239106, 239107, 239108, 239109, 239110, 239111, 239112, 239113, 239114) c6( ) c7(cac, Lu, bCJ); #c1(LOXHD1) c2(XP_006722454) c3(lipoxygenase homology domain-containing protein 1 isoform X7) c4(294713) c5(294714, 294715, 294716, 294717, 294718, 294719) c6( ) c7(cac, Lu, bCJ); #c1(LOXHD1) c2(XP_011524105) c3(lipoxygenase homology domain-containing protein 1 isoform X2) c4(318674, 318675) c5( ) c6( ) c7(cac, Lu, bCJ); #c1(LOXHD1) c2(XP_011524106) c3 (lipoxygenase homology domain-containing protein 1 isoform X3) c4(318676, 318677) c5(318678, 318679, 318680, 318681, 318682, 318683, 318684) c6( ) c7(cac, Lu, bCJ); #c1(LOXL1) c2(NP_005567) c3(lysyl oxidase homolog 1 preproprotein) c4(157546, 157547) c5(157548, 157549, 157550, 157551, 157552, 157553, 157554, 157555, 157556, 157557, 157558, 157559) c6( ) c7(HQ, T, lP, fG, e, lJ, bER, aT, brC, dL, K, eL, dH, aW, dw, cad, xd, W, pZ, vD, Uj, uJ, sa, us); #c1(LOXL2) c2(NP_002309) c3(lysyl oxidase homolog 2 precursor) c4(127499, 127500, 127501, 127502) c5(127503, 127504, 127505, 127506, 127507, 127508, 127509, 127510, 127511, 127512, 127513, 127514, 127515, 127516, 127517, 127518, 127519, 127520, 127521, 127522, 127523, 127524, 127525, 127526) c6( ) c7(T, fG, ht, jx, aA, n, ci, q, hX, jz, Tf, Ot, aT, hu, bc, mu, wr, aW, ff, r, aF, en, g, eY, Qu, uJ, it, X, us); #c1(LOXL3) c2(NP_001276093) c3(lysyl oxidase homolog 3 isoform 2 precursor) c4(107995) c5(107996, 107997, 107998, 107999, 108000, 108001) c6( ) c7(us, aW, Ot, ci, aT); #c1(LOXL3) c2(NP_115992) c3(lysyl oxidase homolog 3 isoform 1 precursor) c4(225133, 225134, 225135) c5(225136, 225137, 225138, 225139, 225140, 225141, 225142, 225143, 225144, 225145, 225146, 225147, 225148, 225149, 225150, 225151, 225152, 225153) c6( ) c7(us, aW, Ot, ci, aT); #c1(LOXL4) c2(NP_115587) c3(lysyl oxidase homolog 4 precursor) c4(223288) c5(223289, 223290, 223291, 223292, 223293, 223294) c6( ) c7(dP, lD, T, aW, ff, aT, dL, ci, en, iK, eL, us, BY); #c1(LPA) c2(NP_005568) c3(apolipoprotein(a) precursor) c4(157560) c5(157561, 157562, 157563, 157564, 157565, 157566, 157567, 157568, 157569, 157570) c6( ) c7(bL, bM, sY, T, dD, ed, eR, ea, e, aw, aA, cz, bB, aj, v, j, ci, ah, vr, eq, aY, V, ss, fK, q, ad, acs, Zu, dY, ec, M, aT, h, wx, dL, mL, jj, dQ, ae, af, eu, hm, eL, A, lR, cH, cW, aW, bvD, md, eH, r, aq, vt, af, eF, t, cM, uC, wA, qa, bm, LB, KT, y, ac, c, bE, X, gX, cj, vy, nT, MJ, dX, cS, m, np, R, aS, dy, fD, cae, B, us); #c1(LPAR1) c2(XP_005251839) c3(lysophosphatidic acid receptor 1 isoform X1) c4(271202, 271203, 271204, 271205, 271206) c5(271207, 271208, 271209, 271210, 271211, 271212, 271213, 271214, 271215, 271216, 271217, 271218, 271219, 271220, 271221, 271222, 271223, 271224, 271225, 271226, 271227, 271228, 271229, 271230, 271231, 271232, 271233, 271234, 271235, 271236, 271237, 271238, 271239, 271240, 271241, 271242, 271243, 271244) c6( ) c7(bL, cZ, T, aC, fG, aA, bB, bS, j, ct, fr, q, ad, eA, aE, aT, lG, K, bU, dd, ae, EN, B, s, aW, bvD, eH, Az, eZ, r, vt, aF, Z, t, eF, bz, hq, LB, bE, m, fD, y, bt, xb, X); #c1(LPAR2) c2(XP_011526723) c3(lysophosphatidic acid receptor 2 isoform X1) c4(321028) c5(321029, 321030, 321031, 321032, 321033, 321034, 321035, 321036, 321037, 321038) c6( ) c7(bL, bM, T, iH, dA, fG, yk, ha, hU, if, cz, bB, fd, v, ci, qN, yp, ye, xY, q, cd, ad, eA, Vp, yn, av, aE, xW, aT, h, xV, bK, K, yb, yh, yj, ae, yi, xX, d, xU, qt, aW, Y, r, yc, yq, cl, t, nC, bz, ik, qa, iP, dR, jq, ok, yf, bP, yl, xZ, bE, z, hL, cj, tl, m, fD, y, X); #c1(LPAR3) c2(XP_011539451) c3(lysophosphatidic acid receptor 3 isoform X1) c4(337617) c5(337618, 337619, 337620, 337621, 337622, 337623, 337624, 337625, 337626, 337627) c6( ) c7(bL, bM, T, dA, if, bB, Ja, j, v, qN, q, ad, dP, aE, pX, aT, h, he, Fg, ae, A, d, aW, Y, ff, r, aq, en, t, bE, dX, m, y, cE, B); #c1(LPAR6) c2(NP_001155970) c3(lysophosphatidic acid receptor 6) c4(61655) c5(61656, 61657, 61658, 61659, 61660, 61661, 61662, 61663, 61664, 61665) c6( ) c7(T, caf, alu, YX, afB, xV, bZx, cag, dL, iV, el); #c1(LPCAT1) c2(NP_079106) c3(lysophosphatidylcholine acyltransferase 1) c4(217043) c5(217044, 217045, 217046, 217047, 217048, 217049, 217050, 217051, 217052, 217053) c6( ) c7(aq, aw, B); #c1(LPCAT2) c2(NP_060309) c3(lysophosphatidylcholine acyltransferase 2) c4(195309) c5(195310, 195311, 195312, 195313, 195314, 195315) c6( ) c7(T, kl, q, r); #c1(LPCAT3) c2(NP_005759) c3(lysophospholipid acyltransferase 5) c4(159128) c5(159129, 159130, 159131, 159132, 159133, 159134) c6( ) c7(B, qa, cz); #c1(LPCAT4) c2(NP_705841) c3(lysophospholipid acyltransferase LPCAT4) c4(246337) c5(246338, 246339, 246340, 246341, 246342, 246343) c6( ) c7(q, r); #c1(LPGAT1) c2(NP_055688) c3(acyl-CoA:lysophosphatidylglycerol acyltransferase 1) c4( ) c5(182782, 182783, 182784, 182785, 182786, 182787, 182788, 182789) c6( ) c7(v, ey, bO); #c1(LPGAT1) c2(XP_005273419) c3(acyl-CoA:lysophosphatidylglycerol acyltransferase 1 isoform X2) c4(284580) c5(284581, 284582) c6( ) c7(v, ey, bO); #c1(LPIN1) c2(NP_001248356) c3 (phosphatidate phosphatase LPIN1 isoform 2) c4(93763, 93764) c5(93765, 93766, 93767, 93768, 93769, 93770, 93771, 93772, 93773, 93774, 93775) c6( ) c7(bL, T, eR, if, e, cz, v, fu, qN, bTg, b, aoA, jy, bk, K, bq, kl, xX, cah, fc, dq, GR, iO, GG, cP, ei, i); #c1(LPIN1) c2(NP_001248357) c3(phosphatidate phosphatase LPIN1 isoform 3) c4(93776, 93777, 93778) c5(93779, 93780, 93781, 93782, 93783, 93784, 93785, 93786, 93787, 93788, 93789, 93790, 93791, 93792, 93793, 93794, 93795) c6( ) c7(bL, T, eR, if, e, cz, v, fu, qN, bTg, b, aoA, jy, bK, K, bq, kl, xX, cah, fc, dq, GR, iO, GG, cP, ei, i); #c1(LPIN1) c2(NP_001248358) c3(phosphatidate phosphatase LPIN1 isoform 4) c4(93796) c5(93797, 93798, 93799, 93800, 93801, 93802, 93803, 93804, 93805, 93806, 93807, 93808) c6( ) c7(bL, T, eR, if, e, cz, v, fu, qN, bTg, b, aoA, jy, bK, K, bq, kl, xX, cah, fc, dq, GR, iO, GG, cP, ei, i); #c1(LPIN1) c2(NP_663731) c3(phosphatidate phosphatase LPIN1 isoform 1) c4(241463) c5(241464, 241465, 241466, 241467, 241468, 241469) c6( ) c7(bL, T, eR, if, e, cz, v, fu, qN, bTg, b, aoA, jy, bK, K, bq, kl, xX, cah, fc, dq, GR, iO, GG, cP, ei, i); #c1(LPIN1) c2(XP_006711932) c3(phosphatidate phosphatase LPIN1 isoform X2) c4( ) c5(286306) c6( ) c7(bL, T, eR, if, e, cz, v, fu, qN, bTg, b, aoA, jy, bk, K, bq, kl, xX, cah, fc, dq, GR, iO, GG, cP, ei, i); #c1(LPIN1) c2(XP_011508636) c3(phosphatidate phosphatase LPIN1 isoform X3) c4(299250, 299251) c5(299252, 299253, 299254, 299255, 299256, 299257) c6( ) c7(bL, T, eR, if, e, cz, v, fu, qN, bTg, b, aoA, jy, bK, K, bq, kl, xX, cah, fc, dq, GR, iO, GG, cP, ei, i); #c1(LPIN1) c2(XP_011508640) c3(phosphatidate phosphatase LPIN1 isoform X6) c4( ) c5(299258) c6( ) c7(bL, T, eR, if, e, cz, v, fu, qN, bTg, b, aoA, jy, bK, K, bq, kl, xX, cah, fc, dq, GR, iO, GG, cP, ei, i); #c1(LPIN2) c2(NP_055461) c3 (phosphatidate phosphatase LPIN2) c4(181317, 181318) c5(181319, 181320, 181321, 181322, 181323, 181324, 181325, 181326, 181327, 181328) c6( ) c7(bL, cai, bvG, aC, adw, oW, dq, v); #c1(LPIN2) c2(XP_005258234) c3(phosphatidate phosphatase LPIN2 isoform X2) c4( ) c5(275593, 275594) c6( ) c7(bL, cai, bvG, aC, adw, oW, dq, v); #c1(LPL) c2(NP_000228) c3(lipoprotein lipase precursor) c4(5098, 5099, 5100, 5101, 5102, 5103, 5104, 5105) c5(5106, 5107, 5108, 5109, 5110, 5111, 5112, 5113, 5114, 5115, 5116, 5117, 5118, 5119, 5120, 5121, 5122, 5123, 5124, 5125, 5126, 5127, 5128, 5129, 5130, 5131, 5132, 5133, 5134, 5135, 5136, 5137, 5138, 5139, 5140, 5141, 5142, 5143, 5144, 5145, 5146, 5147, 5148, 5149, 5150, 5151, 5152, 5153, 5154, 5155, 5156, 5157, 5158, 5159, 5160, 5161, 5162, 5163, 5164, 5165, 5166, 5167, 5168, 5169, 5170, 5171, 5172, 5173, 5174, 5175, 5176, 5177, 5178, 5179, 5180, 5181, 5182, 5183, 5184, 5185, 5186, 5187, 5188, 5189, 5190, 5191, 5192, 5193, 5194, 5195, 5196, 5197, 5198, 5199, 5200, 5201, 5202, 5203, 5204, 5205, 5206, 5207, 5208, 5209, 5210, 5211, 5212, 5213, 5214, 5215, 5216, 5217, 5218, 5219, 5220, 5221, 5222, 5223, 5224, 5225, 5226, 5227, 5228, 5229, 5230, 5231, 5232, 5233, 5234, 5235, 5236, 5237, 5238, 5239, 5240, 5241, 5242, 5243, 5244) c6(5245, 5246, 5247, 5248, 5249, 5250, 5251, 5252, 5253, 5254, 5255, 5256, 5257) c7(G, T, fR, Zz, ea, e, eb, aeu, ci, GK, dY, u, M, iv, GO, qM, dQ, H, aab, ex, eN, Zx, hm, zQ, fc, bYP, hl, vs, g, eF, uC, eM, dc, mx, U, cj, lS, aGv, cam, dD, wu, aw, fg, cQ, Zv, aj, zP, JW, TB, ft, Zu, cc, ec, cU, jy, h, dL, wb, RE, A, Rj, ge, aae, aq, fe, caj, cM, ar, kT, W, UY, dC, cO, Fi, mg, tl, at, GN, B, L, bL, jv, eR, aaa, oe, jx, aA, cz, v, j, avO, so, nS, J, ga, baE, aT, cy, bZl, bq, ae, af, eu, aai, vT, Y, bg, r, iy, wg, ac, c, cak, bpB, mf, aoJ, dy, sY, wi, iW, vr, vG, dG, ah, eq, iG, tK, ad, dZ, av, QJ, ts, oj, vw, wm, sj, uX, nU, cH, cW, Zy, eH, cal, eZ, dq, bHV, Ed, iV, dr, qa, ek, pE, gZ, aS, cP, ei, Zw, X); #c1(LPO) c2(NP_001153574) c3(lactoperoxidase isoform 3 preproprotein) c4(60665) c5(60666, 60667, 60668, 60669, 60670, 60671) c6( ) c7(dB, aW, ki, aT, an, rk); #c1(LPO) c2(NP_006142) c3 (lactoperoxidase isoform 1 preproprotein) c4(162361, 162362, 162363) c5(162364, 162365, 162366, 162367, 162368, 162369, 162370, 162371, 162372, 162373, 162374, 162375) c6( ) c7(dB, aW, ki, al, an, rk); #c1(LPO) c2(XP_011523111) c3(lactoperoxidase isoform X2) c4( ) c5(317617, 317618, 317619, 317620, 317621, 317622) c6( ) c7(dB, aW, ki, al, an, rk); #c1(LPP) c2(NP_001161143) c3(lipoma-preferred partner isoform a) c4(65359) c5(65360, 65361, 65362, 65363, 65364, 65365, 65366, 65367) c6( ) c7(bM, T, aZ, cz, cl, qj, hF, q, o, ad, oB, oj, aT, aHs, K, ae, kl, A, aW, Y, ff, r, hp, en, cl, fq, buo, au, hj, bt); #c1(LPP) c2(NP_001161144) c3(lipoma-preferred partner isoform b) c4(65368, 65369, 65370, 65371, 65372, 65373, 65374, 65375) c5( ) c6( ) c7(bM, T, aZ, cz, cl, qj, hF, q, o, ad, oB, oj, aT, aHs, K, ae, kl, A, aW, Y, ff, r, hp, en, cl, fq, buo, au, hj, bt); #c1(LPP) c2(XP_011511125) c3(lipoma-preferred partner isoform X2) c4(301525) c5(301526, 301527, 301528, 301529, 301530, 301531, 301532, 301533, 301534, 301535) c6( ) c7(bM, T, aZ, cz, cl, qj, hF, q, o, ad, OB, oj, aT, aHs, K, ae, kl, A, aW, Y, ff, r, hp, en, cl, fq, buo, au, hj, bt); #c1(LPP) c2(XP_011511137) c3(lipoma-preferred partner isoform X4) c4( ) c5(301536, 301537, 301538, 301539, 301540, 301541, 301542, 301543, 301544, 301545, 301546, 301547, 301548, 301549, 301550, 301551, 301552, 301553, 301554, 301555, 301556, 301557, 301558, 301559, 301560, 301561, 301562, 301563, 301564, 301565, 301566, 301567, 301568, 301569, 301570, 301571, 301572, 301573, 301574, 301575, 301576, 301577, 301578, 301579, 301580, 301581) c6( ) c7(bM, T, aZ, cz, cl, qj, hF, q, o, ad, oB, oj, aT, aHs, K, ae, kl, A, aW, Y, ff, r, hp, en, cl, fq, buo, au, hj, bt); #c1(LPPR2) c2(NP_001164106) c3(lipid phosphate phosphatase-related protein type 2 isoform 2) c4(66621) c5(66622, 66623, 66624, 66625, 66626, 66627, 66628) c6( ) c7(bjt, can, aBm, agS, bjs); #c1(LPPR2) c2(NP_073574) c3(lipid phosphate phosphatase-related protein type 2 isoform 1) c4(212917) c5(212918, 212919, 212920, 212921, 212922, 212923) c6( ) c7(bjt, can, aBm, agS, bjs); #c1(LPPR4) c2(XP_011540800) c3(lipid phosphate phosphatase-related protein type 4 isoform X1) c4(339359) c5( ) c6( ) c7(T, aE, aW, TG, aT, K); #c1(LPXN) c2(NP_001137467) c3(leupaxin isoform 1) c4(55891) c5(55892, 55893, 55894, 55895, 55896, 55897) c6( ) c7(dE, jh, g, bt, ae, ad); #c1(LPXN) c2(NP_004802) c3(leupaxin isoform 2) c4(149966) c5(149967, 149968, 149969, 149970, 149971, 149972) c6( ) c7(dE, jh, g, bt, ae, ad); #c1(LRAT) c2(NP_001288574) c3(lecithin retinol acyltransferase precursor) c4(113756, 113757) c5(113758, 113759, 113760, 113761, 113762, 113763, 113764, 113765, 113766, 113767, 113768, 113769, 113770, 113771, 113772, 113773) c6( ) c7(T, oQ, lR, Dy, af, Y, nx, aSW, cao, nw, dL, eQ, nC, nk, Nh, caq, OK, ae, cap, ad); #c1(LRBA) c2(NP_006717) c3(lipopolysaccharide-responsive and beige-like anchor protein isoform 2) c4(167385) c5(167386, 167387, 167388, 167389, 167390, 167391) c6( ) c7(n, T, fq, car); #c1(LRBA) c2(XP_005263432) c3(lipopolysaccharide-responsive and beige-like anchor protein isoform X4) c4(278470) c5(278471, 278472, 278473, 278474, 278475, 278476) c6( ) c7(n, T, fq, car); #c1(LRCH1) c2(NP_001157683) c3(leucine-rich repeat and calponin homology domain-containing protein 1 isoform 1) c4(62402) c5(62403, 62404, 62405, 62406, 62407, 62408) c6( ) c7(kh, q, ci, s, r); #c1(LRCH1) c2(NP_001157685) c3(leucine-rich repeat and calponin homology domain-containing protein 1 isoform 3) c4(62409) c5(62410, 62411, 62412, 62413, 62414, 62415) c6( ) c7(kh, q, ci, s, r); #c1(LRCH1) c2(NP_055931) c3(leucine-rich repeat and calponin homology domain-containing protein 1 isoform 2) c4(184505) c5(184506, 184507, 184508, 184509, 184510, 184511) c6( ) c7(kh, q, ci, s, r); #c1(LRCH1) c2(XP_011533315) c3(leucine-rich repeat and calponin homology domain-containing protein 1 isoform X1) c4( ) c5(329463) c6( ) c7(kh, q, ci, s, r); #c1(LRCH4) c2(NP_002310) c3 (leucine-rich repeat and calponin homology domain-containing protein 4 isoform 1) c4(127527, 127528) c5(127529, 127530, 127531, 127532, 127533, 127534, 127535, 127536, 127537, 127538, 127539, 127540) c6( ) c7(T, ib); #c1(LRFN2) c2(NP_065788) c3(leucine-rich repeat and fibronectin type-III domain-containing protein 2 precursor) c4(205435) c5(205436, 205437, 205438, 205439, 205440) c6( ) c7(ht, pN); #c1(LRFN2) c2(XP_011513062) c3(leucine-rich repeat and fibronectin type-III domain-containing protein 2 isoform X2) c4( ) c5(304745) c6( ) c7(ht, pN); #c1(LRFN5) c2(NP_689660) c3(leucine-rich repeat and fibronectin type-III domain-containing protein 5 precursor) c4(243508) c5(243509, 243510, 243511, 243512, 243513, 243514) c6( ) c7(dt); #c1(LRG1) c2(NP_443204) c3(leucine-rich alpha-2-glycoprotein precursor) c4(229914, 229915) c5(229916, 229917, 229918, 229919, 229920, 229921, 229922, 229923, 229924, 229925, 229926, 229927, 229928, 229929, 229930, 229931) c6(229932, 229933, 229934, 229935) c7(T, iV, sw, hl, ji, cl, cm, y); #c1(LRGUK) c2(NP_653249) c3(leucine-rich repeat and guanylate kinase domain-containing protein) c4(239336) c5(239337, 239338, 239339, 239340, 239341, 239342) c6( ) c7(bL, oe, dY); #c1(LRIF1) c2(XP_011540070) c3(ligand-dependent nuclear receptor-interacting factor 1 isoform X2) c4(338368) c5( ) c6( ) c7(eG); #c1(LRIG1) c2(NP_056356) c3(leucine-rich repeats and immunoglobulin-like domains protein 1 precursor) c4(187240, 187241, 187242) c5(187243, 187244, 187245, 187246, 187247, 187248, 187249, 187250, 187251, 187252, 187253, 187254, 187255, 187256, 187257, 187258, 187259, 187260) c6( ) c7(T, j, fr, eA, q, ad, aT, dL, ae, eL, d, s, aW, ff, r, en, cl, as, t, bz, Qt, hL, cj, m); #c1(LRIG3) c2(NP_001129523) c3 (leucine-rich repeats and immunoglobulin-like domains protein 3 isoform 1 precursor) c4(51839) c5(51840, 51841, 51842, 51843, 51844, 51845) c6( ) c7(T, d, aE, dB, aW, awP, aT, hR, it, dL, eA, bC, eL, cd); #c1(LRIG3) c2(NP_700356) c3 (leucine-rich repeats and immunoglobulin-like domains protein 3 isoform 2 precursor) c4(245912) c5(245913, 245914, 245915, 245916, 245917, 245918) c6( ) c7(T, d, aE, dB, aW, awP, aT, hR, it, dL, eA, bC, eL, cd); #c1(LRIT1) c2(NP_056428) c3(leucine-rich repeat, immunoglobulin-like domain and transmembrane domain-containing protein 1 precursor) c4(187552) c5(187553, 187554, 187555, 187556, 187557, 187558) c6( ) c7(Jm, aGm, mR, aZy, Y, aZw, ym, Fg); #c1(LRIT3) c2(NP_940908) c3(leucine-rich repeat, immunoglobulin-like domain and transmembrane domain-containing protein 3 precursor) c4(260090) c5(260091, 260092, 260093, 260094, 260095, 260096) c6( ) c7(cas, oe); #c1(LRIT3) c2(XP_005263036) c3(leucine-rich repeat, immunoglobulin-like domain and transmembrane domain-containing protein 3 isoform X1) c4(278223) c5(278224, 278225, 278226, 278227, 278228, 278229, 278230, 278231, 278232, 278233) c6( ) c7(cas, oe); #c1(LRMP) c2(NP_006143) c3(lymphoid-restricted membrane protein) c4(162376, 162377) c5(162378, 162379, 162380, 162381, 162382, 162383, 162384, 162385, 162386, 162387, 162388, 162389, 162390, 162391, 162392, 162393, 162394, 162395, 162396, 162397) c6( ) c7(aw, av, bz, iu, g); #c1(LRP1) c2(NP_002323) c3(prolow-density lipoprotein receptor-related protein 1 precursor) c4(127541) c5(127542, 127543, 127544, 127545, 127546, 127547) c6( ) c7(bL, T, dD, vl, ea, axd, aw, l, cz, bO, fd, v, cat, j, ci, ur, uu, hF, eq, eA, ad, x, av, iu, ft, xc, kH, aT, cy, h, K, da, ae, af, dx, bA, d, bf, je, cW, aW, Y, Ff, aq, yq, g, cl, t, acD, bz, vo, cm, QA, ck, iM, il, eM, nB, bE, dJ, U, Be, bm, aS, k, m, y, bt, B, L); #c1(LRP10) c2(NP_054764) c3(low-density lipoprotein receptor-related protein 10 precursor) c4(177639, 177640) c5(177641, 177642, 177643, 177644, 177645, 177646, 177647, 177648, 177649, 177650, 177651, 177652) c6( ) c7(cm, af); #c1(LRP12) c2(NP_001129175) c3(low-density lipoprotein receptor-related protein 12 isoform b precursor) c4(51101) c5(51102, 51103, 51104, 51105, 51106, 51107) c6( ) c7(T, bE, aW, FO, aw, FP, aT, r, bB, q, hu, dw, B); #c1(LRP12) c2(NP_038465) c3(low-density lipoprotein receptor-related protein 12 isoform a precursor) c4(176556) c5(176557, 176558, 176559, 176560, 176561, 176562) c6( ) c7(T, bE, aW, FO, aw, FP, aT, r, bB, q, hu, dw, B); #c1(LRP1B) c2(NP_061027) c3(low-density lipoprotein receptor-related protein (B precursor) c4(199043, 199044) c5(199045, 199046, 199047, 199048, 199049, 199050, 199051, 199052, 199053, 199054, 199055, 199056, 199057, 199058, 199059, 199060, 199061, 199062, 199063) c6( ) c7(T, Y, oe, ff, fq, it, vd, fr, en, cm, cau, bA); #c1(LRP1B) c2(XP_011509654) c3(low-density lipoprotein receptor-related protein 1B isoform X1) c4(300228) c5(300229, 300230) c6( ) c7(T, Y, oe, ff, fq, it, vd, fr, en, cm, cau, bA); #c1(LRP2) c2(NP_004516) c3(low-density lipoprotein receptor-related protein 2 precursor) c4(147117) c5(147118, 147119, 147120, 147121, 147122, 147123, 147124, 147125, 147126, 147127, 147128, 147129, 147130, 147131, 147132, 147133, 147134, 147135) c6( ) c7(T, eR, dk, cz, n, hz, oZ, fr, bNM, ad, amB, b, zw, Tl, aE, iX, awd, aT, aU, h, Wm, bq, dQ, H, ae, af, lr, sj, cH, aW, eH, Az, aZi, cav, cm, bKy, es, mO, aPt, aNJ, mG, wk, iS, aSW, va, dy, i, y, ZW, ars, L); #c1(LRP2) c2(XP_011509486) c3(low-density lipoprotein receptor-related protein 2 isoform X2) c4(300065) c5( ) c6( ) c7(T, eR, dk, cz, n, hz, oZ, fr, bNM, ad, amB, b, zw, Tl, aE, iX, awd, aT, aU, h, Wm, bq, dQ, H, ae, af, lr, sj, cH, aW, eH, Az, aZi, cav, cm, bKy, es, mO, aPt, aNJ, mG, wk, iS, aSW, va, dy, i, y, ZW, ars, L); #c1(LRP2) c2(XP_011509487) c3(low-density lipoprotein receptor-related protein 2 isoform X3) c4(300066) c5( ) c6( ) c7(T, eR, dk, cz, n, hz, oZ, fr, bNM, ad, amB, b, zw, Tl, aE, iX, awd, aT, aU, h, Wm, bq, dd, H, ae, af, lr, sj, cH, aW, eH, Az, aZi, cav, cm, bKy, es, m, aPt, aNJ, mG, wk, iS, aSW, va, dy, i, y, ZW, ars, L); #c1(LRP2BP) c2(NP_060879) c3(LRP2-binding protein) c4(198281, 198282, 198283) c5(198284, 198285, 198286, 198287, 198288, 198289, 198290, 198291, 198292, 198293, 198294, 198295, 198296, 198297, 198298, 198299, 198300, 198301, 198302, 198303, 198304, 198305) c6( ) c7(rx); #c1(LRP4) c2(NP_002325) c3(low-density lipoprotein receptor-related protein 4 precursor) c4(127548) c5(127549, 127550, 127551, 127552, 127553, 127554) c6( ) c7(D, hM, xA, zw, nX, xj, aDT, jh, cj, aUt, zU, aUs, cm, af, aUv, bA); #c1(LRP4) c2(XP_011518404) c3(low-density lipoprotein receptor-related protein 4 isoform X1) c4( ) c5(312384) c6( ) c7(D, hM, xA, zw, nX, xj, aDT, jh, cj, aUt, zU, aUs, cm, af, aUv, bA); #c1(LRP4) c2(XP_011518406) c3(low-density lipoprotein receptor-related protein 4 isoform X3) c4( ) c5(312385) c6( ) c7(D, hM, xA, zw, nX, xj, aDT, jh, cj, aUt, zU, aUs, cm, af, aUv, bA); #c1(LRP5) c2(NP_002326) c3(low-density lipoprotein receptor-related protein 5 isoform 1 precursor) c4(127555) c5(127556, 127557, 127558, 127559, 127560, 127561) c6( ) c7(DS, T, dD, dA, ea, zs, aw, aDE, jh, cz, fd, aj, cax, j, v, bX, eq, azk, cay, sg, J, ad, bsH, zw, no, A, bfJ, ari, caw, dL, bYN, K, caz, ae, af, bA, aRR, hi, kl, mR, lR, byj, caB, cH, bf, are, Tc, eH, r, bHg, byl, anW, akb, eF, HI, iV, aSb, dr, zV, bF, Ta, arB, W, nb, ac, yE, YE, bbY, bm, iS, at, Hs, k, ei, y, vt, aAO, caA); #c1(LRP5) c2(XP_011543333) c3(low-density lipoprotein receptor-related protein 5 isoform X4) c4( ) c5(342127) c6( ) c7(DS, T, dD, dA, ea, zs, aw, aDE, jh, cz, fd, aj, cax, j, v, bX, eq, azk, cay, sg, J, ad, bsH, zw, no, A, bfJ, ari, caw, dL, bYN, K, caz, ae, af, bA, aRR, hi, kl, mR, lR, byj, caB, cH, bf, are, Tc, eH, r, bHg, byl, anW, akb, eF, HI, iV, aSb, dr, zV, bF, Ta, arB, W, nb, ac, yE, YE, bbY, bm, iS, at, Hs, k, ei, y, vt, aAO, caA); #c1(LRP6) c2(NP_002327) c3(low-density lipoprotein receptor-related protein 6 precursor) c4(127562, 127563) c5(127564, 127565, 127566, 127567, 127568, 127569, 127570, 127571, 127572, 127573, 127574, 127575) c6( ) c7(DS, T, j, azk, aT, dL, af, bA, aRR, eG, A, kj, aW, caD, are, r, iV, caC, arB, kP, W, aAS, yE, dO, vy, at, ei, it, B); #c1(LRPAP1) c2(NP_002328) c3(alpha-2-macroglobulin receptor-associated protein precursor) c4(127576) c5(127577, 127578, 127579, 127580, 127581, 127582) c6( ) c7(bL, T, dk, aw, caE, aA, fr, ci, ad, afb, Vp, l, py, u, BR, dL, ae, af, qk, bA, bFd, ak, nY, t, bz, pb, Uk, ac, nB, caF, U, Be, m, gT, B, y); #c1(LRPPRC) c2(NP_573566) c3(leucine-rich PPR motif-containing protein, mitochondrial precursor) c4(234539) c5(234540, 234541, 234542, 234543, 234544, 234545, 234546, 234547, 234548, 234549, 234550, 234551) c6( ) c7(bL, T, lT, aD, aw, aA, fd, caG, v, bX, qN, hF, cd, ai, x, jz, aE, rw, Yo, aT, h, ji, K, an, ae, af, ajU, jf, yo, aW, Y, aGS, hl, fe, aq, lo, aF, yq, g, t, bmj, dr, ck, pc, dB, mx, U, acJ, m, k, fD, y, bt, B); #c1(LRPPRC) c2(XP_011530775) c3(leucine-rich PPR motif-containing protein, mitochondrial isoform X1) c4(326810) c5( ) c6( ) c7(bL, T, lT, aD, aw, aA, fd, caG, v, bX, qN, hF, cd, ai, x, jz, aE, rw, Yo, aT, h, ji, K, an, ae, af, ajU, jf, yo, aW, Y, aGS, hl, fe, aq, lo, aF, yq, g, t, bmj, dr, ck, pc, dB, mx, U, acJ, m, k, fD, y, bt, B); #c1(LRR1) c2(NP_689542) c3(leucine-rich repeat protein 1 isoform 1) c4(243048, 243049) c5(243050, 243051, 243052, 243053, 243054, 243055, 243056, 243057, 243058, 243059, 243060, 243061) c6( ) c7(yl, qj, pC); #c1(LRR1) c2(NP_982292) c3(leucine-rich repeat protein 1 isoform 3) c4(263293) c5(263294, 263295, 263296, 263297, 263298, 263299, 263300) c6( ) c7(yl, qj, pC); #c1(LRRC1) c2(NP_060684) c3(leucine-rich repeat-containing protein 1) c4(197151, 197152) c5(197153, 197154, 197155, 197156, 197157, 197158, 197159, 197160, 197161, 197162, 197163, 197164) c6( ) c7(cz); #c1(LRRC15) c2(NP_001128529) c3(leucine-rich repeat-containing protein 15 isoform a precursor) c4(50200, 50201) c5(50202, 50203, 50204, 50205, 50206, 50207, 50208, 50209, 50210, 50211, 50212, 50213, 50214, 50215, 50216, 50217) c6( ) c7(T, Yn, fR, qt, aW, aT, AO, af); #c1(LRRC15) c2(NP_570843) c3(leucine-rich repeat-containing protein 15 isoform b precursor) c4( ) c5(234295) c6( ) c7(T, Yn, fR, qt, aW, aT, AD, af); #c1(LRRC16A) c2(NP_001167448) c3(leucine-rich repeat-containing protein 16A isoform 2) c4(68920) c5(68921, 68922, 68923, 68924, 68925, 68926) c6( ) c7(nD); #c1(LRRC16A) c2(NP_060110) c3(leucine-rich repeat-containing protein 16A isoform 1) c4(194227) c5(194228, 194229, 194230, 194231, 194232, 194233) c6( ) c7(nD); #c1(LRRC16A) c2(XP_011513035) c3(leucine-rich repeat-containing protein 16A isoform X3) c4(304636) c5( ) c6( ) c7(nD); #c1(LRRC16A) c2(XP_011513039) c3(leucine-rich repeat-containing protein 16A isoform X7) c4( ) c5(304637, 304638, 304639, 304640, 304641, 304642) c6( ) c7(nD); #c1(LRRC16B) c2(NP_612369) c3(leucine-rich repeat-containing protein 16B) c4(236284, 236285) c5(236286, 236287, 236288, 236289, 236290, 236291, 236292, 236293, 236294, 236295, 236296, 236297) c6( ) c7(kl); #c1(LRRC16B) c2(XP_011535602) c3(leucine-rich repeat-containing protein 16B isoform X1) c4( ) c5(332857, 332858) c6( ) c7(kl); #c1(LRRC17) c2(NP_005815) c3(leucine-rich repeat-containing protein 17 isoform 2 precursor) c4(159640) c5(159641, 159642, 159643, 159644, 159645, 159646, 159647, 159648, 159649, 159650, 159651, 159652, 159653, 159654, 159655) c6(159656, 159657, 159658, 159659) c7(ck); #c1(LRRC17) c2(XP_005250165) c3(leucine-rich repeat-containing protein 17 isoform X1) c4(270137, 270138) c5(270139, 270140, 270141, 270142, 270143, 270144, 270145, 270146, 270147) c6(270148, 270149) c7(ck); #c1(LRRC18) c2(XP_011538131) c3(leucine-rich repeat-containing protein 18 isoform X1) c4(335699, 335700) c5(335701, 335702, 335703, 335704, 335705, 335706, 335707, 335708, 335709, 335710, 335711, 335712) c6( ) c7(fq, eZ); #c1(LRRC20) c2(NP_001265141) c3(leucine-rich repeat-containing protein 20 isoform 1) c4(97855, 97856) c5(97857, 97858, 97859, 97860, 97861, 97862, 97863, 97864, 97865, 97866, 97867, 97868, 97869, 97870, 97871, 97872) c6( ) c7(j, eZ); #c1(LRRC20) c2(NP_060675) c3(leucine-rich repeat-containing protein 20 isoform 3) c4(197088) c5(197089, 197090, 197091, 197092, 197093, 197094, 197095, 197096, 197097, 197098) c6( ) c7(j, eZ); #c1(LRRC20) c2(NP_060709) c3(leucine-rich repeat-containing protein 20 isoform 2) c4(197316) c5(197317, 197318, 197319, 197320, 197321, 197322, 197323, 197324, 197325, 197326) c6( ) c7(j, eZ); #c1(LRRC26) c2(NP_001013675) c3(leucine-rich repeat-containing protein 26 precursor) c4(25702) c5(25703, 25704, 25705, 25706, 25707, 25708) c6( ) c7(aW, T, ae, ad, aT); #c1(LRRC30) c2(NP_001099051) c3(leucine-rich repeat-containing protein 30) c4(41789) c5(41790, 41791, 41792, 41793, 41794, 41795) c6( ) c7(cm); #c1(LRRC32) c2(XP_005273959) c3(leucine-rich repeat-containing protein 32 isoform X1) c4(284878, 284879) c5(284880, 284881, 284882, 284883, 284884, 284885, 284886, 284887, 284888, 284889, 284890, 284891, 284892, 284893, 284894, 284895, 284896, 284897, 284898, 284899) c6( ) c7(Xk, aDR, alx, aDQ, oc); #c1(LRRC37A) c2(NP_055649) c3(leucine-rich repeat-containing protein 37A precursor) c4(182528) c5(182529, 182530, 182531, 182532, 182533, 182534, 182535, 182536, 182537, 182538) c6( ) c7(nA); #c1(LRRC37A) c2(XP_005257936) c3(leucine-rich repeat-containing protein 37A isoform X5) c4( ) c5(275434) c6( ) c7(nA); #c1(LRRC37A) c2(XP_006725343) c3(leucine-rich repeat-containing protein 37A isoform X8) c4(296769, 296770, 296771, 296772, 296773) c5(296774, 296775, 296776, 296777, 296778, 296779, 296780, 296781, 296782, 296783, 296784, 296785, 296786, 296787, 296788, 296789, 296790, 296791, 296792, 296793, 296794, 296795, 296796, 296797, 296798, 296799, 296800, 296801, 296802, 296803) c6( ) c7(nA); #c1(LRRC37A) c2(XP_011544667) c3(leucine-rich repeat-containing protein 37A isoform X7) c4(343762) c5( ) c6( ) c7(nA); #c1(LRRC37B) c2(NP_443120) c3(leucine-rich repeat-containing protein 378 precursor) c4(229520, 229521) c5(229522, 229523, 229524, 229525, 229526, 229527, 229528, 229529, 229530, 229531, 229532, 229533, 229534, 229535, 229536, 229537, 229538, 229539) c6( ) c7(nX, xb, Az, xt); #c1(LRRC37B) c2(XP_011522563) c3(leucine-rich repeat-containing protein 378 isoform X1) c4(317159) c5(317160, 317161, 317162, 317163, 317164, 317165) c6( ) c7(nX, xb, Az, xt); #c1(LRRC37B) c2(XP_011522570) c3(leucine-rich repeat-containing protein 378 isoform X6) c4(317166) c5( ) c6( ) c7(nX, xb, Az, xt); #c1(LRRC39) c2(XP_011538980) c3 (leucine-rich repeat-containing protein 39 isoform X1) c4(337021) c5(337022, 337023, 337024, 337025, 337026, 337027, 337028, 337029, 337030, 337031) c6( ) c7(cc, af, K); #c1(LRRC4) c2(XP_011514763) c3 (leucine-rich repeat-containing protein 4 isoform X1) c4(307417, 307418, 307419) c5(307420, 307421, 307422, 307423, 307424, 307425, 307426, 307427, 307428, 307429, 307430, 307431, 307432, 307433, 307434, 307435, 307436, 307437) c6( ) c7(es, d, c, iA, dB, aW, aT, ae, eA, LS, ad, Qt); #c1(LRRC49) c2(NP_060161) c3 (leucine-rich repeat-containing protein 49 isoform 2) c4(194532) c5(194533, 194534, 194535, 194536, 194537, 194538) c6( ) c7(aW, aT); #c1(LRRC49) c2(XP_011520018) c3(leucine-rich repeat-containing protein 49 isoform X1) c4(314899) c5(314900, 314901, 314902, 314903, 314904, 314905) c6( ) c7(aW, aT); #c1(LRRC4B) c2(XP_011525822) c3(leucine-rich repeat-containing protein 48 isoform X1) c4(320221, 320222) c5(320223, 320224, 320225, 320226, 320227, 320228, 320229, 320230, 320231, 320232, 320233, 320234) c6( ) c7(fq); #c1(LRRC4C) c2(NP_001245348) c3(leucine-rich repeat-containing protein 4C precursor) c4(93183, 93184) c5(93185, 93186, 93187, 93188, 93189, 93190, 93191, 93192, 93193, 93194, 93195, 93196, 93197, 93198, 93199, 93200, 93201, 93202, 93203, 93204, 93205, 93206, 93207, 93208) c6( ) c7(bM, sj); #c1(LRRC52) c2(NP_001005214) c3(leucine-rich repeat-containing protein 52 precursor) c4(21155) c5(21156, 21157, 21158, 21159, 21160, 21161) c6( ) c7(bA, cz); #c1(LRRC59) c2(NP_060979) c3(leucine-rich repeat-containing protein 59) c4(198895) c5(198896, 198897, 198898, 198899, 198900, 198901) c6( ) c7(bF, bE, bB, ey, bC, bD, bz); #c1(LRRC6) c2(NP_036604) c3(protein tilB homolog) c4(174762, 174763) c5(174764, 174765, 174766, 174767, 174768, 174769, 174770, 174771, 174772, 174773, 174774, 174775, 174776, 174777, 174778, 174779) c6( ) c7(caH, BV, Pf, MG); #c1(LRRC61) c2(XP_011514822) c3(leucine-rich repeat-containing protein 61 isoform X1) c4(307490) c5(307491, 307492, 307493, 307494, 307495, 307496, 307497, 307498, 307499, 307500, 307501, 307502, 307503, 307504) c6( ) c7(fe); #c1(LRRC63) c2(NP_001269389) c3(leucine-rich repeat-containing protein 63) c4(101554) c5(101555, 101556, 101557, 101558, 101559, 101560) c6( ) c7(wL); #c1(LRRC69) c2(NP_001123362) c3(leucine-rich repeat-containing protein 69) c4(47040, 47041, 47042) c5(47043, 47044, 47045, 47046, 47047, 47048, 47049, 47050, 47051, 47052, 47053, 47054, 47055, 47056, 47057, 47058, 47059, 47060) c6( ) c7(cm, cd); #c1(LRRC7) c2(XP_011540138) c3(leucine-rich repeat-containing protein 7 isoform X1) c4(338485, 338486) c5(338487, 338488, 338489, 338490, 338491, 338492, 338493, 338494, 338495, 338496, 338497, 338498, 338499, 338500) c6( ) c7(A, xs, cz, aU, cu, eW); #c1(LRRC74A) c2(NP_919263) c3(leucine-rich repeat-containing protein 74A) c4(258355, 258356) c5(258357, 258358, 258359, 258360, 258361, 258362, 258363, 258364, 258365, 258366, 258367) c6( ) c7(j); #c1(LRRC74A) c2(XP_011534782) c3(leucine-rich repeat-containing protein 74A isoform X5) c4(331225) c5(331226, 331227, 331228, 331229, 331230, 331231) c6( ) c7(j); #c1(LRRC74A) c2(XP_011534789) c3(leucine-rich repeat-containing protein 74A isoform X12) c4( ) c5(331232) c6( ) c7(j); #c1(LRRC8A) c2(NP_062540) c3(volume-regulated anion channel subunit LRRC8A) c4(201874) c5(201875, 201876, 201877, 201878, 201879, 201880, 201881, 201882, 201883, 201884, 201885, 201886, 201887, 201888) c6( ) c7(Ze, cal); #c1(LRRC8C) c2(XP_011540584) c3(volume-regulated anion channel subunit LRRC8C isoform X1) c4( ) c5(339094, 339095, 339096, 339097, 339098) c6( ) c7(bK); #c1(LRRCC1) c2(NP_208325) c3(leucine-rich repeat and coiled-coil domain-containing protein 1) c4(228526, 228527) c5(228528, 228529, 228530, 228531, 228532, 228533, 228534, 228535, 228536, 228537, 228538, 228539) c6( ) c7(wR, Zf, Za, C); #c1(LRRCC1) c2(XP_005251375) c3(leucine-rich repeat and coiled-coil domain-containing protein 1 isoform X2) c4(270928) c5(270929, 270930, 270931, 270932, 270933) c6( ) c7(wR, Zf, Za, C); #c1(LRRCC1) c2(XP_005251379) c3(leucine-rich repeat and coiled-coil domain-containing protein 1 isoform X5) c4( ) c5(270934) c6( ) c7(wR, Zf, Za, C); #c1(LRRFIP1) c2(NP_001131022) c3 (leucine-rich repeat flightless-interacting protein 1 isoform 1) c4(52510, 52511) c5(52512, 52513, 52514, 52515, 52516, 52517, 52518, 52519, 52520, 52521, 52522, 52523) c6( ) c7(d, ac, dO, aw, r, ci, q); #c1(LRRFIP1) c2(NP_001131024) c3(leucine-rich repeat flightless-interacting protein 1 isoform 3) c4(52524) c5(52525, 52526, 52527, 52528, 52529, 52530) c6( ) c7(d, ac, dO, aw, r, ci, q); #c1(LRRFIP1) c2(NP_001131025) c3(leucine-rich repeat flightless-interacting protein 1 isoform 5) c4(52531) c5(52532, 52533, 52534, 52535, 52536, 52537) c6( ) c7(d, ac, dO, aw, r, ci, q); #c1(LRRFIP1) c2(NP_004726) c3 (leucine-rich repeat flightless-interacting protein 1 isoform 4) c4(149109) c5(149110, 149111, 149112, 149113, 149114, 149115) c6( ) c7(d, ac, dO, aw, r, ci, q); #c1(LRRFIP1) c2(XP_011510466) c3(leucine-rich repeat flightless-interacting protein 1 isoform X41) c4(300978) c5( ) c6( ) c7(d, ac, dO, aw, r, ci, q); #c1(LRRFIP1) c2(XP_011510467) c3(leucine-rich repeat flightless-interacting protein 1 isoform X42) c4( ) c5(300979, 300980, 300981, 300982, 300983) c6( ) c7(d, ac, dO, aw, r, ci, q); #c1(LRRFIP2) c2(NP_001127841) c3(leucine-rich repeat flightless-interacting protein 2 isoform 3) c4(49360) c5(49361, 49362, 49363, 49364, 49365, 49366) c6( ) c7(T, BO); #c1(LRRFIP2) c2(NP_006300) c3(leucine-rich repeat flightless-interacting protein 2 isoform 1) c4(163818) c5(163819, 163820, 163821, 163822, 163823, 163824, 163825, 163826, 163827, 163828) c6( ) c7(T, BO); #c1(LRRFIP2) c2(NP_060194) c3(leucine-rich repeat flightless-interacting protein 2 isoform 2) c4(194718) c5(194719, 194720, 194721, 194722, 194723, 194724, 194725, 194726, 194727, 194728, 194729, 194730, 194731, 194732, 194733, 194734, 194735, 194736, 194737, 194738) c6( ) c7(T, BO); #c1(LRRFIP2) c2(XP_006713466) c3(leucine-rich repeat flightless-interacting protein 2 isoform X28) c4(287166) c5( ) c6( ) c7(T, BO); #c1(LRRK1) c2(XP_011520315) c3(leucine-rich repeat serine/threonine-protein kinase 1 isoform X1) c4(315157, 315158) c5(315159, 315160, 315161, 315162, 315163, 315164, 315165, 315166, 315167, 315168, 315169, 315170, 315171, 315172, 315173, 315174, 315175, 315176) c6( ) c7(alS, an); #c1(LRRK1) c2(XP_011520317) c3(leucine-rich repeat serine/threonine-protein kinase 1 isoform X3) c4(315177) c5( ) c6( ) c7(alS, an); #c1(LRRK1) c2(XP_011520320) c3(leucine-rich repeat serine/threonine-protein kinase 1 isoform X5) c4(315178) c5(315179, 315180, 315181, 315182, 315183, 315184, 315185, 315186, 315187, 315188, 315189) c6( ) c7(alS, an); #c1(LRRK1) c2(XP_011520321) c3(leucine-rich repeat serine/threonine-protein kinase 1 isoform X6) c4(315190) c5(315191) c6( ) c7(alS, an); #c1(LRRK2) c2(NP_940980) c3(leucine-rich repeat serine/threonine-protein kinase 2) c4(260248) c5(260249, 260250, 260251, 260252, 260253, 260254, 260255, 260256, 260257, 260258, 260259) c6( ) c7(T, caJ, Ol, yk, Nt, GC, fr, alS, xg, dP, AW, Hx, Gt, WY, HC, bUn, Wk, aT, aU, ji, K, an, he, af, hm, ak, asp, cak, aW, WQ, fe, aaE, dg, cm, acD, xw, GB, aeb, bb, acC, c, Ji, U, nw, act, abd, aen, NN); #c1(LRRN1) c2(NP_065924) c3(leucine-rich repeat neuronal protein 1 precursor) c4(206286) c5(206287, 206288, 206289, 206290, 206291, 206292) c6( ) c7(cm, T, oe, gv); #c1(LRRN2) c2(NP_006329) c3(leucine-rich repeat neuronal protein 2 precursor) c4(164055) c5(164056, 164057, 164058, 164059, 164060, 164061, 164062, 164063, 164064, 164065, 164066, 164067, 164068, 164069) c6( ) c7(bM, fw, d, eA, qy); #c1(LRRN3) c2(NP_001093128) c3(leucine-rich repeat neuronal protein 3 precursor) c4(39801) c5(39802, 39803, 39804, 39805, 39806, 39807, 39808, 39809, 39810, 39811, 39812, 39813, 39814, 39815) c6( ) c7(cm, T, oe, VX); #c1(LRRN4) c2(NP_689824) c3(leucine-rich repeat neuronal protein 4 precursor) c4(244044) c5(244045, 244046, 244047, 244048, 244049, 244050) c6( ) c7(T, gv, ib); #c1(LRRTM1) c2(NP_849161) c3(leucine-rich repeat transmembrane neuronal protein 1 precursor) c4(254337, 254338, 254339, 254340) c5(254341, 254342, 254343, 254344, 254345, 254346, 254347, 254348, 254349, 254350, 254351, 254352, 254353, 254354, 254355, 254356, 254357, 254358, 254359, 254360, 254361, 254362, 254363, 254364, 254365, 254366, 254367, 254368, 254369, 254370, 254371, 254372, 254373, 254374, 254375, 254376, 254377) c6( ) c7(bM, Fn, c, aA, sv, gv, yA, jb); #c1(LRRTM2) c2(NP_056379) c3(leucine-rich repeat transmembrane neuronal protein 2 precursor) c4(187349) c5(187350, 187351, 187352, 187353, 187354, 187355) c6( ) c7(bw); #c1(LRRTM3) c2(NP_821079) c3(leucine-rich repeat transmembrane neuronal protein 3 precursor) c4(253214) c5(253215, 253216, 253217, 253218, 253219, 253220, 253221, 253222, 253223, 253224) c6( ) c7(fK, VX, af); #c1(LRRTM4) c2(NP_001128217) c3(leucine-rich repeat transmembrane neuronal protein 4 isoform a precursor) c4(49726) c5(49727, 49728, 49729, 49730, 49731, 49732, 49733, 49734, 49735, 49736) c6( ) c7(fq); #c1(LRRTM4) c2(NP_079269) c3(leucine-rich repeat transmembrane neuronal protein 4 isoform b precursor) c4(217642) c5(217643, 217644, 217645, 217646, 217647, 217648, 217649) c6( ) c7(fq); #c1(LRRTM4) c2(XP_011531417) c3 (leucine-rich repeat transmembrane neuronal protein 4 isoform X1) c4( ) c5(327507, 327508, 327509, 327510, 327511, 327512) c6( ) c7(fq); #c1(LRRTM4) c2(XP_011531419) c3(leucine-rich repeat transmembrane neuronal protein 4 isoform X3) c4(327513) c5(327514, 327515, 327516, 327517, 327518, 327519) c6( ) c7(fq); #c1(LRSAM1) c2(NP_001177652) c3(E3 ubiquitin-protein ligase LRSAM1 isoform 2) c4(72823) c5(72824, 72825, 72826, 72827, 72828, 72829) c6( ) c7(eR, qb, cal, kH, lo, bU, dr); #c1(LRSAM1) c2(NP_612370) c3(E3 ubiquitin-protein ligase LRSAM1 isoform 1) c4(236298) c5(236299, 236300, 236301, 236302, 236303, 236304, 236305, 236306, 236307, 236308, 236309, 236310, 236311, 236312) c6( ) c7(BR, qb, cal, KH, lo, bu, dr); #c1(LRTM1) c2(NP_001291318) c3(leucine-rich repeat and transmembrane domain-containing protein 1 isoform 2) c4(115662) c5(115663, 115664, 115665, 115666, 115667) c6( ) c7(bL, aw, j); #c1(LRTM1) c2(NP_065729) c3(leucine-rich repeat and transmembrane domain-containing protein 1 isoform 1 precursor) c4(205107) c5(205108, 205109, 205110, 205111, 205112, 205113, 205114) c6( ) c7(bL, aw, j); #c1(LRTOMT) c2(NP_001138779) c3(leucine-rich repeat-containing protein 51 isoform LRTOMT(b) c4(57662) c5(57663, 57664, 57665, 57666, 57667, 57668, 57669, 57670, 57671, 57672) c6( ) c7(aW, caM, Bh, aT); #c1(LRTOMT) c2(NP_001138781) c3(transmembrane O-methyltransferase isoform LRTOMT2a) c4(57673) c5(57674, 57675, 57676, 57677, 57678, 57679, 57680, 57681, 57682, 57683) c6( ) c7(aW, caM, Bh, aT); #c1(LRTOMT) c2(NP_001138782) c3(transmembrane O-methyltransferase isoform LRTOMT2b) c4(57684) c5(57685, 57686, 57687, 57688, 57689, 57690) c6( ) c7(aW, caM, Bh, aT); #c1(LRTOMT) c2(NP_001192067) c3(leucine-rich repeat-containing protein 51 isoform LRTOMT1c) c4(81910) c5(81911, 81912, 81913, 81914, 81915, 81916) c6( ) c7(aW, caM, Bh, aT); #c1(LRTOMT) c2(NP_001258400) c3(leucine-rich repeat-containing protein 51 isoform LRTOMT1d) c4( ) c5(96057, 96058, 96059, 96060, 96061, 96062, 96063, 96064, 96065, 96066, 96067, 96068, 96069, 96070, 96071, 96072, 96073, 96074) c6( ) c7(aW, caM, Bh, aT); #c1(LRTOMT) c2(NP_660352) c3(leucine-rich repeat-containing protein 51 isoform LRTOMT1a) c4(241054, 241055, 241056, 241057) c5(241058, 241059, 241060, 241061, 241062, 241063, 241064, 241065, 241066, 241067) c6( ) c7(aW, caM, Bh, aT); #c1(LSAMP) c2(NP_002329) c3(limbic system-associated membrane protein preproprotein) c4(127583) c5(127584, 127585, 127586, 127587, 127588, 127589) c6( ) c7(T, dD, bO, aA, bS, j, eq, fr, ww, py, ds, cU, h, XZ, d, tb, s, eH, apn, r, Ni, lo, aF, eF, awi, hq, alm, aDX, ig, awx, AH, iu); #c1(LSAMP) c2(XP_005247511) c3(limbic system-associated membrane protein isoform X1) c4(267856) c5(267857, 267858, 267859, 267860, 267861, 267862) c6( ) c7(T, dD, bO, aA, bS, j, eq, fr, ww, py, ds, cU, h, XZ, d, tb, s, eH, apn, r, Ni, lo, aF, eF, awi, hq, alm, aDX, ig, awx, AH, iu); #c1(LSM1) c2(NP_055277) c3(U6 snRNA-associated Sm-like protein LSm1) c4(180230) c5(180231, 180232, 180233, 180234, 180235, 180236) c6( ) c7(ae, aC, ib, aE, aW, aT, bX, bz, ad); #c1(LSM11) c2(NP_775762) c3(U7 snRNA-associated Sm-like protein LSm11) c4(250210) c5(250211, 250212, 250213, 250214, 250215, 250216) c6( ) c7(aw); #c1(LSM2) c2(NP_067000) c3(U6 snRNA-associated Sm-like protein LSm2) c4(208217) c5(208218, 208219, 208220, 208221, 208222, 208223, 208224, 208225, 208226, 208227, 208228, 208229, 208230, 208231, 208232, 208233, 208234, 208235, 208236, 208237, 208238) c6( ) c7(zT, T, eW, eZ, aT, kf, uo, h, mK, yZ, aGX, aVK, yU, f, nC, caN, Ro); #c1(LSM3) c2(NP_055278) c3(UG snRNA-associated Sm-like protein LSm3) c4(180237) c5(180238, 180239, 180240, 180241, 180242, 180243) c6( ) c7(Ea); #c1(LSM4) c2(NP_036453) c3(UG snRNA-associated Sm-like protein LSm4 isoform 1) c4(174000) c5(174001, 174002, 174003, 174004, 174005, 174006, 174007, 174008, 174009, 174010) c6( ) c7(T, aA, fr, eA, ad, av, ft, zw, aE, pX, aT, K, ae, gj, aW, aF, g, cm, t, bz, iK, es, eM, iA, m, y); #c1(LSM5) c2(NP_036454) c3(UG snRNA-associated Sm-like protein LSm5 isoform a) c4(174011) c5(174012, 174013, 174014, 174015, 174016, 174017) c6( ) c7(kl); #c1(LSM6) c2(NP_009011) c3(UG snRNA-associated Sm-like protein LSm6) c4(170223) c5(170224, 170225, 170226, 170227, 170228, 170229, 170230, 170231, 170232, 170233, 170234, 170235, 170236, 170237, 170238, 170239, 170240, 170241) c6( ) c7(UF); #c1(LSM7) c2(NP_057283) c3(U6 snRNA-associated Sm-like protein LSm7) c4(189846) c5(189847, 189848, 189849, 189850, 189851, 189852) c6( ) c7(T); #c1(LSP1) c2(NP_001013273) c3(lymphocyte-specific protein 1 isoform 2) c4(25465, 25466, 25467, 25468, 25469) c5(25470, 25471, 25472, 25473, 25474, 25475, 25476, 25477, 25478, 25479, 25480, 25481, 25482, 25483, 25484, 25485, 25486, 25487, 25488, 25489, 25490, 25491, 25492, 25493, 25494, 25495, 25496, 25497, 25498, 25499, 25500) c6( ) c7(T, aE, up, PE, Y, aW, iW, aT, lS, as, iV, bz, eJ); #c1(LSP1) c2(NP_001229861) c3(lymphocyte-specific protein 1 isoform 3) c4(85476) c5(85477, 85478, 85479, 85480, 85481, 85482, 85483, 85484) c6( ) c7(T, aE, up, PE, Y, aW, iW, aT, lS, as, iV, bz, eJ); #c1(LSP1) c2(NP_002330) c3(lymphocyte-specific protein 1 isoform 1) c4(127590) c5(127591, 127592, 127593, 127594, 127595, 127596, 127597, 127598, 127599, 127600, 127601) c6( ) c7(T, aE, up, PE, Y, aW, iW, aT, lS, as, iV, bz, eJ); #c1(LSR) c2(NP_001247418) c3(lipolysis-stimulated lipoprotein receptor isoform 4) c4(93496) c5(93497, 93498, 93499, 93500, 93501, 93502, 93503, 93504) c6(93505, 93506) c7(T, zQ, AD, bE, aW, aT, bB, tv, tu, ci, zP, X); #c1(LSR) c2(NP_001247419) c3(lipolysis-stimulated lipoprotein receptor isoform 5) c4(93507) c5(93508, 93509, 93510, 93511, 93512, 93513, 93514, 93515) c6(93516, 93517) c7(T, zQ, AO, bE, aW, aT, bB, tv, tu, ci, zP, X); #c1(LSR) c2(NP_057009) c3(lipolysis-stimulated lipoprotein receptor isoform 1) c4(188452) c5(188453, 188454, 188455, 188456, 188457, 188458, 188459, 188460, 188461) c6(188462, 188463) c7(T, zQ, AO, bE, aW, aT, bB, tv, tu, ci, zP, X); #c1(LSR) c2(NP_991403) c3(lipolysis-stimulated lipoprotein receptor isoform 2) c4(263458) c5(263459, 263460, 263461, 263462, 263463, 263464, 263465, 263466) c6(263467, 263468) c7(T, zQ, AD, bE, aW, aT, bB, tv, tu, ci, zP, X); #c1(LSR) c2(NP_991404) c3(lipolysis-stimulated lipoprotein receptor isoform 3) c4(263469) c5(263470, 263471, 263472, 263473, 263474, 263475, 263476, 263477) c6(263478, 263479) c7(T, zQ, AD, bE, aW, aT, bB, tv, tu, ci, zP, X); #c1(LSR) c2(XP_005259037) c3(lipolysis-stimulated lipoprotein receptor isoform X1) c4(275925) c5(275926, 275927, 275928, 275929, 275930, 275931, 275932, 275933) c6(275934, 275935) c7(T, zQ, AD, bE, aW, aT, bB, tv, tu, ci, zP, X); #c1(LSS) c2(NP_001001438) c3(lanosterol synthase isoform 1) c4(18491) c5(18492, 18493, 18494, 18495, 18496, 18497, 18498, 18499, 18500, 18501, 18502, 18503, 18504, 18505, 18506, 18507) c6( ) c7(hT, ff, agF, en, du, ZW); #c1(LSS) c2(NP_001138908) c3(lanosterol synthase isoform 2) c4(58130) c5(58131, 58132, 58133, 58134, 58135, 58136) c6( ) c7(hT, ff, agF, en, du, ZW); #c1(LSS) c2(NP_001138909) c3(lanosterol synthase isoform 3) c4(58137) c5( ) c6( ) c7(hT, ff, agF, en, du, ZW); #c1(LST1) c2(NP_995309) c3(leukocyte-specific transcript 1 protein isoform 2) c4(263480, 263481) c5(263482, 263483, 263484, 263485, 263486, 263487, 263488, 263489, 263490, 263491, 263492, 263493, 263494, 263495, 263496, 263497, 263498, 263499, 263500, 263501, 263502, 263503, 263504, 263505, 263506, 263507, 263508, 263509, 263510, 263511, 263512, 263513, 263514, 263515, 263516, 263517, 263518, 263519, 263520, 263521, 263522, 263523, 263524, 263525, 263526, 263527, 263528, 263529, 263530, 263531, 263532, 263533, 263534, 263535, 263536, 263537, 263538, 263539, 263540, 263541, 263542, 263543, 263544, 263545, 263546, 263547, 263548, 263549, 263550, 263551, 263552, 263553, 263554, 263555, 263556, 263557) c6( ) c7(A, fR, sQ, dV, cr, uo, eZ, ut, Hs, pD, lQ, C, aiJ, vk); #c1(LST1) c2(NP_995310) c3(leukocyte-specific transcript 1 protein isoform 3) c4(263558, 263559) c5(263560, 263561, 263562, 263563, 263564, 263565, 263566, 263567, 263568, 263569, 263570, 263571, 263572, 263573, 263574, 263575, 263576, 263577, 263578, 263579, 263580, 263581, 263582, 263583, 263584, 263585, 263586, 263587, 263588, 263589, 263590, 263591, 263592, 263593, 263594, 263595, 263596, 263597, 263598, 263599, 263600, 263601, 263602, 263603, 263604, 263605, 263606, 263607, 263608, 263609, 263610, 263611, 263612, 263613, 263614, 263615, 263616, 263617, 263618, 263619, 263620, 263621, 263622, 263623, 263624, 263625, 263626, 263627, 263628, 263629, 263630, 263631, 263632, 263633, 263634, 263635, 263636, 263637, 263638, 263639, 263640, 263641, 263642, 263643, 263644, 263645, 263646, 263647, 263648, 263649, 263650, 263651, 263652, 263653, 263654, 263655, 263656, 263657, 263658, 263659, 263660, 263661, 263662, 263663, 263664, 263665, 263666, 263667, 263668, 263669, 263670, 263671, 263672, 263673, 263674, 263675, 263676, 263677, 263678, 263679, 263680, 263681, 263682, 263683, 263684, 263685, 263686, 263687, 263688, 263689, 263690, 263691, 263692, 263693) c6( ) c7(A, fR, sQ, dV, cr, uo, eZ, ut, Hs, pD, lQ, C, aiJ, vk); #c1(LST1) c2(NP_99531 l) c3(leukocyte-specific transcript 1 protein isoform 4) c4(263694, 263695, 263696, 263697, 263698) c5(263699, 263700, 263701, 263702, 263703, 263704, 263705, 263706, 263707, 263708, 263709, 263710, 263711, 263712, 263713, 263714, 263715, 263716, 263717, 263718, 263719, 263720, 263721, 263722, 263723, 263724, 263725, 263726, 263727, 263728, 263729, 263730, 263731, 263732, 263733, 263734, 263735, 263736, 263737, 263738, 263739, 263740, 263741, 263742, 263743, 263744, 263745, 263746, 263747, 263748, 263749, 263750, 263751, 263752, 263753, 263754, 263755, 263756, 263757, 263758, 263759, 263760, 263761, 263762, 263763, 263764, 263765, 263766, 263767, 263768, 263769, 263770, 263771, 263772, 263773, 263774, 263775, 263776, 263777, 263778, 263779, 263780, 263781, 263782, 263783, 263784, 263785, 263786, 263787, 263788) c6( ) c7(A, fR, sQ, dV, cr, uo, eZ, ut, Hs, pD, lQ, C, aiJ, vk); #c1(LST1) c2(XP_006715272) c3(leukocyte-specific transcript 1 protein isoform X2) c4(289195, 289196) c5(289197, 289198, 289199, 289200, 289201, 289202, 289203, 289204, 289205, 289206, 289207, 289208, 289209, 289210, 289211, 289212, 289213, 289214, 289215, 289216, 289217, 289218, 289219, 289220, 289221, 289222, 289223, 289224, 289225, 289226, 289227, 289228, 289229, 289230, 289231, 289232, 289233, 289234, 289235, 289236, 289237, 289238, 289239, 289240, 289241, 289242, 289243, 289244, 289245, 289246, 289247, 289248, 289249, 289250, 289251, 289252, 289253, 289254, 289255, 289256, 289257, 289258, 289259, 289260, 289261, 289262, 289263, 289264, 289265, 289266, 289267) c6( ) c7(A, fR, s, dV, cr, uo, eZ, ut, Hs, pD, lQ, C, aiJ, vk); #c1(LST1) c2(XP_006715273) c3(leukocyte-specific transcript 1 protein isoform X3) c4(289268, 289269, 289270, 289271) c5(289272, 289273, 289274, 289275, 289276, 289277, 289278, 289279, 289280, 289281, 289282, 289283, 289284, 289285, 289286, 289287, 289288, 289289, 289290, 289291, 289292, 289293, 289294, 289295, 289296, 289297, 289298, 289299, 289300, 289301, 289302, 289303, 289304, 289305, 289306, 289307, 289308, 289309, 289310, 289311, 289312, 289313, 289314, 289315, 289316, 289317, 289318, 289319, 289320, 289321, 289322, 289323, 289324, 289325, 289326, 289327, 289328, 289329, 289330, 289331, 289332, 289333, 289334, 289335, 289336, 289337, 289338, 289339, 289340, 289341, 289342, 289343, 289344, 289345, 289346, 289347, 289348, 289349, 289350, 289351, 289352, 289353, 289354, 289355, 289356, 289357, 289358, 289359, 289360, 289361, 289362, 289363, 289364, 289365, 289366, 289367, 289368, 289369, 289370, 289371, 289372, 289373, 289374, 289375, 289376, 289377, 289378, 289379, 289380, 289381, 289382, 289383, 289384, 289385, 289386, 289387, 289388, 289389, 289390, 289391, 289392, 289393, 289394, 289395, 289396, 289397, 289398, 289399, 289400, 289401, 289402, 289403, 289404, 289405, 289406, 289407, 289408, 289409, 289410, 289411, 289412, 289413, 289414, 289415, 289416, 289417, 289418, 289419, 289420, 289421, 289422, 289423, 289424, 289425, 289426, 289427, 289428, 289429, 289430, 289431, 289432, 289433, 289434, 289435, 289436, 289437, 289438, 289439, 289440, 289441) c6( ) c7(A, fR, sQ, dV, cr, uo, eZ, ut, Hs, pD, lQ, C, aiJ, vk); #c1(LST1) c2(XP_011513216) c3(leukocyte-specific transcript 1 protein isoform X1) c4(305437) c5(305438, 305439, 305440, 305441, 305442, 305443, 305444, 305445, 305446, 305447, 305448, 305449, 305450, 305451, 305452, 305453, 305454, 305455, 305456, 305457, 305458, 305459, 305460, 305461, 305462, 305463, 305464, 305465, 305466) c6( ) c7(A, fR, sQ, dV, cr, uo, eZ, ut, Hs, pD, lQ, C, aiJ, vk); #c1(LTA) c2(XP_011512920) c3(lymphotoxin-alpha isoform X1) c4(304285, 304286) c5(304287, 304288, 304289, 304290, 304291, 304292, 304293, 304294, 304295, 304296, 304297, 304298, 304299, 304300, 304301, 304302, 304303, 304304, 304305, 304306, 304307, 304308, 304309, 304310, 304311, 304312, 304313, 304314, 304315, 304316, 304317, 304318, 304319, 304320) c6( ) c7(bM, T, os, fR, ea, if, e, eb, fd, cl, ur, qj, aMB, bHS, AW, aE, lu, bW, fS, M, dV, Ds, dQ, hH, DY, eN, aiJ, hm, bzH, aa, bn, aW, axu, hl, kt, pD, g, eE, bw, uC, y, fT, aDs, mx, vy, cj, k, eg, qR, dD, sw, aw, ht, n, aj, BF, aBl, uE, qN, qW, cd, ai, eh, eW, aPb, Mo, aMj, cU, Ne, jy, dL, aaA, wb, h, eV, aDp, bEg, A, sp, ge, fe, yq, CA, vo, cM, Wt, ar, kT, hk, mg, bcd, hL, uz, at, eo, fl, gg, bL, Dw, jv, fM, eR, AL, jx, eX, aA, cz, tc, fF, v, j, C, bX, wy, V, pd, q, J, o, zn, ga, mP, hb, nX, jZ, ld, qp, aT, vH, qG, aDt, rz, ae, caR, af, dl, bA, eu, aFS, hM, s, il, Y, apK, r, iy, aPf, en, bu, cl, pb, vk, OC, iM, sn, c, ac, ig, AH, caP, aYx, EY, fD, byK, gT, bt, ao, sY, aD, em, iW, jh, oR, oZ, dG, uu, jE, eq, mC, ad, cal, dZ, dP, hX, bPu, av, F, wD, oW, Os, hD, JK, vB, WW, ul, asn, lU, je, cW, ff, cr, eZ, dq, aF, cal, hp, iV, Z, bz, Hv, qa, ek, LB, gk, axs, yE, z, dO, sG, gZ, Hs, aS, ei, lf, iu, hv, pC); #c1(LTA) c2(XP_011545552) c3 (lymphotoxin-alpha isoform X1) c4(344305, 344306) c5(344307, 344308, 344309, 344310, 344311, 344312, 344313, 344314, 344315, 344316, 344317, 344318, 344319, 344320, 344321, 344322) c6( ) c7(bM, T, os, fR, ea, if, e, eb, fd, cl, ur, qj, aMB, bHS, AW, aE, lu, bW, fS, M, dV, Ds, de, hH, DY, eN, aiJ, hm, bzH, aa, bn, aW, axu, hl, kt, pD, g, eE, bw, uC, y, fT, aDs, mx, vy, cj, k, eg, qR, dD, sw, aw, ht, n, aj, BF, aBl, uE, qN, qW, cd, ai, eh, eW, aPb, Mo, aMj, cU, Ne, jy, dL, aaA, wb, h, eV, aDp, bEg, A, sp, ge, fe, yq, CA, vo, cM, Wt, ar, kT, hk, mg, bcd, hl, uz, at, eo, fl, gg, bL, Dw, jv, fM, eR, AL, jx, eX, aA, cz, tc, fF, v, j, C, bX, wy, V, pd, q, J, o, zn, ga, mP, hb, nX, jZ, ld, qp, aT, vH, qG, aDt, rz, ae, caR, af, dl, bA, eu, aFS, hM, s, iU, Y, apk, r, iy, aPf, en, bu, cl, pb, vk, QC, iM, sn, c, ac, ig, AH, caP, aYx, EY, fD, byK, gT, bt, ao, sY, aD, em, iW, jh, OR, oZ, dG, uu, jE, eq, mC, ad, cal, dZ, dP, hX, bPu, av, F, wD, oW, Os, HD, JK, vB, WW, ul, asn, lU, je, cW, ff, cr, eZ, dq, aF, cal, hp, iV, Z, bz, Hv, qa, ek, LB, gk, axs, yE, z, do, sG, gZ, Hs, aS, ei, lf, iu, hv, pC); #c1(LTA) c2(XP_011546545) c3(lymphotoxin-alpha isoform X1) c4(345348, 345349) c5(345350, 345351, 345352, 345353, 345354, 345355, 345356, 345357, 345358, 345359, 345360, 345361, 345362, 345363, 345364, 345365, 345366, 345367, 345368, 345369, 345370, 345371, 345372, 345373, 345374, 345375, 345376, 345377, 345378, 345379, 345380, 345381, 345382, 345383) c6( ) c7(bM, T, os, fR, ea, if, e, eb, fd, cl, ur, qj, aMB, bHS, AW, aE, lu, bW, fS, M, dV, Ds, dQ, hH, DY, eN, aiJ, hm, bzH, aa, bn, aW, axu, hl, kt, pD, g, eE, bw, uC, y, fT, aDs, mx, vy, cj, k, eg, qR, dD, sw, aw, ht, n, aj, BF, aBl, uE, qN, qW, cd, ai, eh, eW, aPb, Mo, aMj, cU, Ne, jy, dL, aaA, wb, h, eV, aDp, bEg, A, sp, ge, fe, yq, CA, vo, cM, Wt, ar, kT, hk, mg, bcd, hl, uz, at, eo, fl, gg, bL, Dw, jv, fM, eR, AL, jx, ex, aA, cz, tc, fF, v, j, C, bX, wy, V, pd, q, J, o, zn, ga, mP, hb, nX, jZ, ld, qp, aT, vH, qG, aDt, rz, ae, caR, af, dl, bA, eu, aFS, hM, s, il, Y, apK, r, iy, aPf, en, bu, cl, pb, vk, OC, iM, sn, c, ac, ig, AH, caP, aYx, EY, fD, byK, gT, bt, ao, sY, aD, em, iW, jh, oR, oZ, dG, uu, jE, eq, mC, ad, cal, dZ, dP, hX, bPu, av, F, wD, oW, Os, hD, JK, vB, WW, uQ, asn, lU, je, cW, ff, cr, eZ, dq, af, caQ, hp, iV, Z, bz, Hv, qa, ek, LB, gk, axs, yE, z, dO, sG, gZ, Hs, aS, ei, lf, iu, hv, pC); #c1(LTA4H) c2(NP_000886) c3 (leukotriene A-4 hydrolase isoform 1) c4(16837) c5(16838, 16839, 16840, 1684), 16842, 16843) c6( ) c7(bM, dD, ea, e, aw, bS, j, ct, ah, caS, pM, eq, ad, Hr, AW, dY, aE, M, ae, eu, iU, ff, mQ, caU, en, g, Z, sK, ou, cT, acC, ac, ig, ei, cal); #c1(LTA4H) c2(NP_001243572) c3(leukotriene A-4 hydrolase isoform 2) c4(91072) c5(91073, 91074, 91075, 91076, 91077, 91078) c6( ) c7(bM, dD, ea, e, aw, bS, j, ct, ah, caS, pM, eq, ad, Hr, AW, dY, aE, M, ae, eu, iU, ff, mQ, caU, en, g, Z, sK, ou, cT, acC, ac, ig, ei, cal); #c1(LTA4H) c2(NP_001243573) c3(leukotriene A-4 hydrolase isoform 3) c4(91079) c5(91080, 91081, 91082, 91083, 91084, 91085) c6( ) c7(bM, dD, ea, e, aw, bS, j, ct, ah, caS, pM, eq, ad, Hr, AW, dY, aE, M, ae, eu, ill, ff, mQ, caU, en, g, Z, sK, ou, cT, acC, ac, ig, ei, caT); #c1(LTB) c2(NP_002332) c3(lymphotoxin-beta isoform a) c4(127602) c5(127603, 127604, 127605, 127606, 127607, 127608, 127609, 127610, 127611, 127612, 127613, 127614, 127615, 127616, 127617, 127618, 127619, 127620, 127621, 127622, 127623, 127624, 127625, 127626, 127627, 127628, 127629, 127630) c6( ) c7(bM, T, dD, aw, eX, jh, j, BF, eq, ai, dZ, x, aE, dV, h, eV, K, eu, iU, caU, eZ, aq, KM, DF, ck, TA, rR, acC, gt, ac, cj, at, k, fD, eg, y, bt, B, hv); #c1(LTB) c2(NP_033666) c3(lymphotoxin-beta isoform b) c4(172310) c5(172311, 172312, 172313, 172314, 172315) c6( ) c7(bM, T, dD, aw, eX, jh, j, BF, eq, ai, dZ, x, aE, dV, h, eV, K, eu, iU, caU, eZ, aq, KM, DF, ck, TA, rR, acC, gt, ac, cj, at, k, fD, eg, y, bt, B, hv); #c1(LTB4R) c2(NP_001137391) c3 (leukotriene B4 receptor 1) c4(55613, 55614) c5(55615, 55616, 55617, 55618, 55619, 55620, 55621, 55622, 55623, 55624, 55625, 55626, 55627, 55628, 55629, 55630, 55631, 55632, 55633, 55634) c6( ) c7(bM, aC, dD, id, Ke, ea, fG, eq, C, ai, wa, dY, aE, h, alO, dL, af, eu, bf, hl, t, ac, bm, aS, m, fD, y); #c1(LTB4R2) c2(NP_062813) c3(leukotriene B4 receptor 2) c4(202056, 202057, 202058, 202059) c5(202060, 202061, 202062, 202063, 202064, 202065, 202066, 202067, 202068, 202069, 202070, 202071, 202072, 202073, 202074, 202075, 202076, 202077, 202078, 202079, 202080, 202081, 202082, 202083, 202084, 202085, 202086, 202087, 202088) c6( ) c7(bM, T, dD, id, ea, eq, eJ, ai, ad, dY, aE, aT, dL, eL, ae, Nr, aW, Xm, t, DF, Nq, ac, z, y); #c1(LTBP1) c2(NP_000618) c3(latent-transforming growth factor beta-binding protein 1 isoform LTBP-1S precursor) c4(13972, 13973) c5(13974, 13975, 13976, 13977, 13978, 13979, 13980, 13981, 13982, 13983, 13984, 13985) c6( ) c7(Gl, T, ht, al, lj, hy, j, F, aT, Gw, an, r, t, op, LB, ac, vA, bm, tV, m, Gu, hS); #c1(LTBP1) c2(NP_001159736) c3(latent-transforming growth factor beta-binding protein 1 isoform 4 precursor) c4(64649) c5(64650, 64651, 64652, 64653, 64654, 64655) c6( ) c7(Gl, T, ht, al, lj, hy, j, F, aT, Gw, an, r, t, op, LB, ac, vA, bm, tV, m, Gu, hS); #c1(LTBP1) c2(NP_001159737) c3(latent-transforming growth factor beta-binding protein 1 isoform 3 precursor) c4(64656) c5(64657, 64658, 64659, 64660, 64661, 64662) c6( ) c7(Gl, T, ht, al, lj, hy, j, F, aT, Gw, an, r, t, op, LB, ac, vA, bm, tV, m, Gu, hS); #c1(LTBP1) c2(NP_996826) c3(latent-transforming growth factor beta-binding protein 1 isoform LTBP-1L precursor) c4(264665) c5(264666, 264667, 264668, 264669, 264670, 264671) c6( ) c7(Gl, T, ht, al, lj, hy, j, F, aT, Gw, an, r, t, op, LB, ac, vA, bm, tV, m, Gu, hS); #c1(LTBP1) c2(XP_005264375) c3(latent-transforming growth factor beta-binding protein 1 isoform X11) c4( ) c5(278830, 278831, 278832, 278833, 278834, 278835) c6( ) c7(Gl, T, ht, al, lj, hy, j, F, aT, Gw, an, r, t, op, LB, ac, vA, bm, tV, m, Gu, hS); #c1(LTBP1) c2(XP_011531155) c3(latent-transforming growth factor beta-binding protein 1 isoform X1) c4(327117) c5(327118, 327119, 327120, 327121, 327122, 327123) c6( ) c7(Gl, T, ht, al, lj, hy, j, F, aT, Gw, an, r, t, op, LB, ac, vA, bm, tV, m, Gu, hS); #c1(LTBP1) c2(XP_011531159) c3 (latent-transforming growth factor beta-binding protein 1 isoform X5) c4(327124) c5( ) c6( ) c7(Gl, T, ht, al, lj, hy, j, F, aT, Gw, an, r, t, op, LB, ac, vA, bm, tV, m, Gu, hS); #c1(LTBP2) c2(NP_000419) c3(latent-transforming growth factor beta-binding protein 2 precursor) c4(8782) c5(8783, 8784, 8785, 8786, 8787, 8788) c6( ) c7(vl, al, jx, fd, lj, cd, q, F, Eb, ib, Er, caX, aNM, dH, Y, vj, caW, dw, op, cad, ac, pZ, vD, vA, DC, caV, DE, JJ, adU, it, B, hS); #c1(LTBP3) c2(NP_001123616) c3(latent-transforming growth factor beta-binding protein 3 isoform 1 precursor) c4(47830, 47831, 47832) c5(47833, 47834, 47835, 47836, 47837, 47838, 47839, 47840, 47841, 47842, 47843, 47844, 47845, 47846, 47847, 47848, 47849, 47850, 47851, 47852, 47853, 47854, 47855, 47856, 47857, 47858, 47859, 47860, 47861, 47862, 47863, 47864, 47865, 47866, 47867, 47868, 47869, 47870, 47871, 47872) c6( ) c7(vl, al, fd, lj, cd, q, F, Eb, ib, Er, caX, aNM, dH, Y, Hz, Az, dw, cad, caY, bm, DE, adU, it, hS); #c1(LTBP3) c2(NP_001157738) c3(latent-transforming growth factor beta-binding protein 3 isoform 3) c4( ) c5(62470, 62471, 62472, 62473, 62474, 62475, 62476, 62477) c6( ) c7(vl, al, fd, lj, cd, q, F, Eb, ib, Er, caX, aNM, dH, Y, Hz, Az, dw, cad, caY, bm, DE, adU, it, hS); #c1(LTBP3) c2(NP_066548) c3(latent-transforming growth factor beta-binding protein 3 isoform 2 precursor) c4(207344) c5(207345, 207346, 207347, 207348, 207349, 207350, 207351, 207352) c6( ) c7(vl, al, fd, lj, cd, q, F, Eb, ib, Er, caX, aNM, dH, Y, Hz, Az, dw, cad, caY, bm, DE, adU, it, hS); #c1(LTBP3) c2(XP_011543334) c3(latent-transforming growth factor beta-binding protein 3 isoform X1) c4(342128, 342129) c5( ) c6( ) c7(vl, al, fd, lj, cd, q, F, Eb, ib, Er, caX, aNM, dH, Y, Hz, Az, dw, cad, caY, bm, DE, adU, it, hS); #c1(LTBP3) c2(XP_011543335) c3(latent-transforming growth factor beta-binding protein 3 isoform X2) c4(342130) c5(342131) c6( ) c7(vl, al, fd, lj, cd, q, F, Eb, ib, Er, caX, aNM, dH, Y, Hz, Az, dw, cad, caY, bm, DE, adu, it, hS); #c1(LTBP4) c2(NP_001036009) c3(latent-transforming growth factor beta-binding protein 4 isoform a precursor) c4(33685) c5(33686, 33687, 33688, 33689, 33690, 33691) c6( ) c7(ht, al, ci, q, F, oj, aT, hu, bf, Y, vj, xs, ff, r, uY, en, g, caZ, bm, at, aS, cba, us); #c1(LTBP4) c2(NP_001036010) c3 (latent-transforming growth factor beta-binding protein 4 isoform c precursor) c4(33692) c5(33693, 33694, 33695, 33696, 33697, 33698, 33699) c6( ) c7(ht, al, ci, q, F, oj, aT, hu, bf, Y, vj, xs, ff, r, uY, en, g, caZ, bm, at, aS, cba, us); #c1(LTBP4) c2(NP_003564) c3 (latent-transforming growth factor beta-binding protein 4 isoform b) c4(139202, 139203) c5( ) c6( ) c7(ht, al, ci, q, F, oj, aT, hu, bf, Y, vj, xs, ff, r, uY, en, g, caZ, bm, at, aS, cba, us); #c1(LTBP4) c2(XP_011525679) c3(latent-transforming growth factor beta-binding protein 4 isoform X2) c4( ) c5(320088, 320089, 320090, 320091, 320092, 320093) c6( ) c7(ht, al, ci, q, F, oj, aT, hu, bf, Y, vj, xs, ff, r, uY, en, g, caZ, bm, at, aS, cba, us); #c1(LTBP4) c2(XP_011525683) c3(latent-transforming growth factor beta-binding protein 4 isoform X6) c4(320094) c5(320095, 320096, 320097, 320098, 320099, 320100) c6( ) c7(ht, al, ci, q, F, oj, aT, hu, bf, Y, vj, xs, ff, r, uY, en, g, caZ, bm, at, aS, cba, us); #c1(LTBP4) c2(XP_011525687) c3 (latent-transforming growth factor beta-binding protein 4 isoform X10) c4(320101) c5(320102, 320103, 320104, 320105, 320106) c6( ) c7(ht, al, ci, q, F, oj, aT, hu, bf, Y, vj, xs, ff, r, uY, en, g, caZ, bm, at, aS, cba, us); #c1(LTBP4) c2(XP_011525688) c3(latent-transforming growth factor beta-binding protein 4 isoform X11) c4(320107) c5(320108, 320109, 320110, 320111, 320112, 320113) c6( ) c7(ht, al, ci, q, F, oj, aT, hu, bf, Y, vj, xs, ff, r, uY, en, g, caZ, bm, at, aS, cba, us); #c1(LTBP4) c2(XP_011525689) c3(latent-transforming growth factor beta-binding protein 4 isoform X12) c4( ) c5(320114, 320115, 320116, 320117, 320118, 320119, 320120) c6( ) c7(ht, al, ci, q, F, oj, al, hu, bf, Y, vj, xs, ff, r, uY, en, g, caZ, bm, at, aS, cba, us); #c1(LTBR) c2(NP_001257916) c3(tumor necrosis factor receptor superfamily member 3 isoform 2) c4(95797) c5(95798, 95799, 95800, 95801, 95802) c6( ) c7(00, bM, T, dD, aC, aYZ, ea, dM, df, e, fG, jh, fF, fd, aj, v, eq, ci, C, aFV, bfO, aoj, ai, J, ad, zn, Tz, aE, pe, fh, tT, ju, dV, aMj, aT, cy, hD, lQ, ae, Nr, mR, aCq, iU, agT, A, aW, Y, oV, cr, aw, dN, po, pl, t, f, arJ, dr, aYt, OC, Nq, ck, kT, adz, il, ac, c, Jr, iT, cj, wR, m, y, bt, zA, AN, hv, aHl); #c1(LTBR) c2(NP_002333) c3(tumor necrosis factor receptor superfamily member 3 isoform 1 precursor) c4(127631) c5(127632, 127633, 127634, 127635, 127636, 127637, 127638) c6( ) c7(00, bM, T, dD, aC, aYZ, ea, dM, df, e, fG, jh, fF, fd, aj, v, eq, ci, C, aFV, bfO, aoj, ai, J, ad, zn, Tz, aE, pe, fh, tT, ju, dV, aMj, aT, cy, hD, lQ, ae, Nr, mR, aCq, iU, agT, A, aW, Y, oV, cr, aw, dN, po, pl, t, f, arJ, dr, aYt, OC, Nq, ck, KT, adz, il, ac, c, Jr, iT, cj, wR, m, y, bt, zA, AN, hv, aHl); #c1(LTBR) c2(XP_006719046) c3(tumor necrosis factor receptor superfamily member 3 isoform X2) c4( ) c5(292202) c6( ) c7(00, bM, T, dD, aC, aYZ, ea, dM, df, e, fG, jh, fF, fd, aj, v, eq, ci, C, aFV, bfO, aoj, ai, J, ad, zn, Tz, aE, pe, fh, tT, ju, dV, aMj, aT, cy, hD, lQ, ae, Nr, mR, aCq, iU, agT, A, aW, Y, oV, cr, aw, dN, po, pL, t, f, arJ, dr, aYt, OC, Nq, ck, kT, adz, il, ac, c, Jr, iT, cj, wR, m, y, bt, zA, AN, hv, aHl); #c1(LTC4S) c2(NP_665874) c3(leukotriene C4 synthase) c4(241897) c5(241898, 241899, 241900, 241901, 241902, 241903) c6( ) c7(bL, bM, bjX, e, aw, eX, rf, j, bX, dY, fS, M, Tx, cy, eV, JK, cbb, af, eu, iU, sp, ap, vs, cM, tk, ac, z, hc, JM, at, Tu); #c1(LTF) c2(NP_001186078) c3 (lactotransferrin isoform 2) c4(77050) c5(77051, 77052, 77053, 77054, 77055, 77056) c6( ) c7(bM, sY, ka, T, dA, fR, Dw, aPJ, aKm, n, afK, v, ci, hF, Tz, ad, eW, dP, av, ft, aaU, rw, oW, aT, Ds, ey, ae, bA, le, il, aW, Y, oV, Zf, aq, cl, bBu, iV, uC, bz, aBs, D, eM, ki, wz, Br, uz, mf, X); #c1(LTF) c2(NP_002334) c3(lactotransferrin isoform 1 precursor) c4(127639, 127640, 127641) c5(127642, 127643, 127644, 127645, 127646, 127647, 127648, 127649, 127650, 127651, 127652, 127653) c6( ) c7(bM, sY, ka, T, dA, fR, Dw, aPJ, aKm, n, afK, v, ci, hF, Tz, ad, eW, dP, av, ft, aaU, rw, oW, aT, Ds, ey, ae, bA, le, il, aW, Y, oV, Zf, aq, cl, bBu, iV, uC, bz, aBs, D, eM, ki, wz, Br, uz, mf, X); #c1(LTK) c2(NP_001129157) c3 (leukocyte tyrosine kinase receptor isoform 3 precursor) c4(50938) c5(50939, 50940, 50941, 50942, 50943, 50944) c6( ) c7(ck, T, kl, eZ, bX); #c1(LTK) c2(NP_002335) c3(leukocyte tyrosine kinase receptor isoform 1 precursor) c4(127654) c5(127655, 127656, 127657, 127658, 127659, 127660) c6( ) c7(ck, T, kl, eZ, bX); #c1(LTK) c2(NP_996844) c3(leukocyte tyrosine kinase receptor isoform 2 precursor) c4(264748) c5(264749, 264750, 264751, 264752, 264753, 264754) c6( ) c7(ck, T, kl, eZ, bX); #c1(LTK) c2(XP_011519858) c3(leukocyte tyrosine kinase receptor isoform X1) c4(314598) c5(314599, 314600, 314601, 314602, 314603, 314604) c6( ) c7(ck, T, kl, eZ, bX); #c1(LUC7L) c2(NP_060502) c3(putative RNA-binding protein Luc7-like 1 isoform a) c4(196262) c5(196263, 196264, 196265, 196266, 196267, 196268, 196269, 196270, 196271, 196272, 196273, 196274, 196275, 196276, 196277, 196278, 196279, 196280) c6( ) c7(oW, qe, ER); #c1(LUC7L) c2(NP_958815) c3(putative RNA-binding protein Luc7-like 1 isoform b) c4(262215, 262216) c5(262217) c6( ) c7(oW, qe, ER); #c1(LUC7L) c2(XP_005255486) c3(putative RNA-binding protein Luc7-like 1 isoform X4) c4(273673, 273674) c5(273675, 273676, 273677, 273678, 273679, 273680, 273681, 273682, 273683, 273684, 273685) c6( ) c7(oW, qe, ER); #c1(LUC7L) c2(XP_011520863) c3(putative RNA-binding protein Luc7-like 1 isoform X2) c4( ) c5(315873) c6( ) c7(oW, qe, ER); #c1(LUC7L) c2(XP_011520865) c3(putative RNA-binding protein Luc7-like 1 isoform X3) c4(315874, 315875) c5(315876, 315877, 315878, 315879, 315880, 315881, 315882, 315883, 315884, 315885) c6( ) c7(oW, qe, ER); #c1(LUC7L3) c2(NP_057508) c3(luc7-like protein 3) c4(191192) c5( ) c6( ) c7(bv, T, cF, r, q, ik, cd); #c1(LUC7L3) c2(XP_005257506) c3(luc7-like protein 3 isoform X2) c4(275153, 275154) c5( ) c6( ) c7(bv, T, cF, r, q, ik, cd); #c1(LUC7L3) c2(XP_005257512) c3(luc7-like protein 3 isoform X7) c4( ) c5(275155, 275156, 275157, 275158, 275159, 275160, 275161, 275162, 275163, 275164, 275165, 275166, 275167, 275168, 275169, 275170, 275171, 275172, 275173, 275174, 275175, 275176, 275177, 275178, 275179, 275180, 275181, 275182, 275183, 275184) c6( ) c7(bv, T, cF, r, q, ik, cd); #c1(LUC7L3) c2(XP_006722006) c3(luc7-like protein 3 isoform X6) c4(294539) c5( ) c6( ) c7(bv, T, cF, r, q, ik, cd); #c1(LUM) c2(NP_002336) c3(lumican precursor) c4(127661) c5(127662, 127663, 127664, 127665, 127666, 127667) c6( ) c7(T, asg, aC, eR, zt, if, jx, lL, bB, j, cd, q, eA, eJ, afb, cc, aE, Db, Bc, le, aT, Yi, cp, cW, aW, Y, ev, eH, hl, eZ, r, ff, lo, kp, en, g, eF, bz, D, bE, nG, mg, Be, rx, iu); #c1(LURAP1L) c2(NP_981948) c3(leucine rich adaptor protein 1-like) c4(263184) c5(263185, 263186, 263187, 263188, 263189, 263190) c6( ) c7(j); #c1(LUZP4) c2(NP_057467) c3(leucine zipper protein 4) c4(191116, 191117) c5(191118, 191119, 191120, 191121, 191122, 191123, 191124, 191125, 191126, 191127, 191128) c6( ) c7(WB, fd, bu); #c1(LUZP4) c2(XP_005268400) c3(leucine zipper protein 4 isoform X1) c4(281797) c5(281798, 281799, 281800, 281801, 281802, 281803) c6( ) c7(WB, fd, bu); #c1(LUZP6) c2(NP_001122091) c3(leucine zipper protein 6) c4( ) c5(46441) c6( ) c7(ae, ad); #c1(LVRN) c2(NP_776161) c3(aminopeptidase Q) c4(250902, 250903) c5(250904, 250905, 250906, 250907, 250908, 250909, 250910, 250911, 250912, 250913, 250914, 250915) c6( ) c7(y, Y, jv, uC, cz); #c1(LVRN) c2(XP_011541565) c3(aminopeptidase Q isoform X3) c4(340104) c5(340105, 340106, 340107, 340108, 340109, 340110) c6( ) c7(y, Y, jv, uC, cz); #c1(LXN) c2(NP_064554) c3(latexin) c4(202976) c5(202977, 202978, 202979, 202980, 202981, 202982) c6( ) c7(ID, T, jh, aA, bS, ci, aQF, ad, vN, BY, eA, aow, aT, K, dQ, H, ae, aW, Y, aF, hf, cl, t, op, bN, hq, ck, pH, vA, aiM, hL, dW, m, lC, y); #c1(LY6D) c2(NP_003686) c3(lymphocyte antigen 60 precursor) c4(140185) c5(140186, 140187, 140188, 140189, 140190, 140191) c6( ) c7(ik, g); #c1(LY6E) c2(NP_001120685) c3(lymphocyte antigen GE precursor) c4(44554, 44555) c5(44556, 44557, 44558, 44559, 44560, 44561, 44562, 44563, 44564, 44565, 44566, 44567, 44568, 44569, 44570, 44571, 44572, 44573, 44574, 44575, 44576, 44577, 44578, 44579, 44580, 44581, 44582, 44583, 44584, 44585, 44586, 44587, 44588, 44589, 44590, 44591) c6( ) c7(T, aC, alW, Ol, yY, rf, Nt, lb, GC, Kh, alS, bX, V, Kx, ad, Gt, KB, aV, ds, aU, yZ, alU, aPF, bV, ae, af, alL, cC, rU, eZ, akL, EH, adE, aBs, alT, ac, ky, kB, c, Kv, X, U, nw, Kj, alV, B); #c1(LY6G5B) c2(NP_067044) c3(lymphocyte antigen 6 complex locus protein G5b precursor) c4(208595, 208596, 208597, 208598, 208599) c5(208600, 208601, 208602, 208603, 208604, 208605, 208606, 208607, 208608, 208609, 208610, 208611, 208612, 208613, 208614, 208615, 208616, 208617, 208618, 208619, 208620, 208621, 208622, 208623, 208624, 208625, 208626, 208627, 208628, 208629, 208630, 208631, 208632, 208633, 208634, 208635, 208636, 208637, 208638, 208639, 208640, 208641, 208642, 208643, 208644, 208645, 208646, 208647, 208648, 208649, 208650, 208651, 208652, 208653, 208654, 208655, 208656, 208657, 208658, 208659, 208660, 208661, 208662, 208663, 208664, 208665, 208666, 208667, 208668, 208669, 208670, 208671, 208672, 208673, 208674, 208675, 208676, 208677, 208678, 208679, 208680, 208681, 208682) c6(208683, 208684, 208685, 208686, 208687, 208688, 208689, 208690, 208691, 208692, 208693, 208694, 208695, 208696, 208697, 208698) c7(A, ac, aw, e, eZ, j, eu); #c1(LY6G5C) c2(NP_079538) c3(lymphocyte antigen 6 complex locus protein G5c precursor) c4(218941, 218942, 218943, 218944) c5(218945, 218946, 218947, 218948, 218949, 218950, 218951, 218952, 218953, 218954, 218955, 218956, 218957, 218958, 218959, 218960, 218961, 218962, 218963, 218964, 218965, 218966, 218967, 218968, 218969, 218970, 218971, 218972, 218973, 218974, 218975, 218976, 218977, 218978, 218979, 218980, 218981, 218982, 218983, 218984, 218985, 218986, 218987, 218988, 218989, 218990, 218991, 218992, 218993, 218994, 218995, 218996, 218997, 218998, 218999, 219000, 219001, 219002, 219003, 219004, 219005, 219006, 219007, 219008, 219009, 219010, 219011, 219012, 219013, 219014, 219015, 219016, 219017, 219018, 219019, 219020, 219021, 219022, 219023, 219024, 219025, 219026, 219027, 219028, 219029, 219030, 219031, 219032, 219033, 219034, 219035, 219036, 219037, 219038, 219039, 219040, 219041, 219042, 219043, 219044, 219045, 219046, 219047, 219048, 219049, 219050, 219051, 219052, 219053, 219054, 219055, 219056, 219057, 219058, 219059, 219060, 219061, 219062, 219063, 219064, 219065, 219066) c6(219067, 219068, 219069, 219070, 219071, 219072, 219073, 219074, 219075, 219076, 219077, 219078, 219079, 219080, 219081, 219082, 219083, 219084, 219085, 219086, 219087, 219088, 219089, 219090, 219091, 219092, 219093, 219094, 219095, 219096, 219097, 219098, 219099, 219100, 219101, 219102, 219103, 219104, 219105, 219106, 219107, 219108) c7(y, W, eZ); #c1(LYGG6C) c2(NP_079537) c3(lymphocyte antigen 6 complex locus protein G6c precursor) c4(218906) c5(218907, 218908, 218909, 218910, 218911, 218912, 218913, 218914, 218915, 218916, 218917, 218918, 218919, 218920, 218921, 218922, 218923, 218924, 218925, 218926, 218927, 218928, 218929, 218930, 218931, 218932, 218933, 218934, 218935, 218936, 218937, 218938, 218939, 218940) c6( ) c7(y, ah, eZ, fd); #c1(LY6G6C) c2(XP_011546609) c3(lymphocyte antigen 6 complex locus protein G6c isoform X1) c4(345434) c5( ) c6( ) c7(y, ah, eZ, fd); #c1(LY6G6F) c2(NP_001003693) c3(lymphocyte antigen 6 complex locus protein G6f precursor) c4(19904, 19905, 19906, 19907, 19908) c5(19909, 19910, 19911, 19912, 19913, 19914, 19915, 19916, 19917, 19918, 19919, 19920, 19921, 19922, 19923, 19924, 19925, 19926, 19927, 19928, 19929, 19930, 19931, 19932, 19933, 19934, 19935, 19936, 19937, 19938, 19939, 19940, 19941, 19942, 19943, 19944, 19945, 19946, 19947, 19948, 19949, 19950, 19951, 19952, 19953, 19954, 19955, 19956, 19957, 19958, 19959, 19960, 19961, 19962, 19963) c6( ) c7(WY, W, eZ); #c1(LY6K) c2(NP_059997) c3(lymphocyte antigen GK isoform 1 precursor) c4(193575, 193576) c5(193577, 193578, 193579, 193580, 193581, 193582, 193583, 193584, 193585, 193586, 193587, 193588, 193589, 193590, 193591, 193592, 193593, 193594, 193595) c6(193596, 193597, 193598, 193599) c7(T, eM, aW, ff, ht, bu, aA, aT, dL, v, ci, en, hu, aF, iK, eL); #c1(LY75) c2(NP_002340) c3(lymphocyte antigen 75 precursor) c4(127675) c5(127676, 127677, 127678, 127679, 127680, 127681) c6( ) c7(Y, eX, mx, gZ, h, eV, m, t, pd, cd); #c1(LY75-CD302) c2(NP_001185688) c3(LY75-CD302 fusion protein isoform 1 precursor) c4(76037) c5(76038, 76039, 76040, 76041, 76042, 76043, 76044, 76045, 76046, 76047) c6( ) c7(Y, mx, gZ, h, m, t, cd); #c1(LY75-CD302) c2(NP_001185689) c3(LY75-CD302 fusion protein isoform 2 precursor) c4(76048) c5(76049, 76050, 76051, 76052, 76053, 76054, 76055, 76056, 76057, 76058) c6( ) c7(Y, mx, gZ, h, m, t, cd); #c1(LY86) c2(NP_004262) c3(lymphocyte antigen 86 precursor) c4(144680) c5(144681, 144682, 144683, 144684, 144685, 144686, 144687, 144688, 144689, 144690, 144691, 144692, 144693, 144694) c6(144695, 144696) c7(bM, j, lS, up, pN); #c1(LY9) c2(NP_001028839) c3(T-lymphocyte surface antigen Ly-9 isoform b) c4(30679) c5(30680, 30681, 30682, 30683, 30684, 30685) c6( ) c7(B, eZ, fd); #c1(LY9) c2(NP_001248385) c3(T-lymphocyte surface antigen Ly-9 isoform c) c4( ) c5(93919, 93920) c6( ) c7(B, eZ, fd); #c1(LY9) c2(NP_001248386) c3(T-lymphocyte surface antigen Ly-9 isoform d precursor) c4(93921, 93922) c5(93923, 93924, 93925, 93926, 93927, 93928, 93929, 93930, 93931, 93932, 93933, 93934, 93935) c6( ) c7(B, eZ, fd); #c1(LY9) c2(NP_002339) c3(T-lymphocyte surface antigen Ly-9 isoform a precursor) c4(127668) c5(127669, 127670, 127671, 127672, (27673, 127674) c6( ) c7(B, eZ, fd); #c1(LY9) c2(XP_011507853) c3(T-lymphocyte surface antigen Ly-9 isoform X4) c4(298000) c5(298001, 298002, 298003, 298004, 298005) c6( ) c7(B, eZ, fd); #c1(LY9) c2(XP_011507854) c3(T-lymphocyte surface antigen Ly-9 isoform X5) c4(298006) c5(298007, 298008, 298009, 298010, 298011, 298012, 298013, 298014, 298015) c6( ) c7(B, eZ, fd); #c1(LY9) c2(XP_011507856) c3(T-lymphocyte surface antigen Ly-9 isoform X7) c4(298016) c5(298017, 298018, 298019, 298020, 298021, 298022) c6( ) c7(B, eZ, fd); #c1(LY9) c2(XP_011507857) c3(T-lymphocyte surface antigen Ly-9 isoform X8) c4(298023) c5(298024, 298025, 298026, 298027, 298028) c6( ) c7(B, eZ, fd); #c1(LY96) c2(NP_001182726) c3(lymphocyte antigen 96 isoform 2 precursor) c4(75161) c5(75162, 75163, 75164, 75165, 75166, 75167, 75168, 75169) c6(75170, 75171) c7(oO, bL, d, fR, uR, mQ, aA, fF, aFr, fe, ci, aDE, C, q); #c1(LY96) c2(NP_056179) c3(lymphocyte antigen 96 isoform 1 precursor) c4(186158) c5(186159, 186160, 186161, 186162, 186163, 186164, 186165, 186166) c6(186167, 186168) c7(oO, bL, d, fR, uR, ml, aA, fF, aFr, fe, ci, aDE, C, q); #c1(LYL1) c2(XP_006722815) c3(protein lyl-1 isoform X2) c4(294890) c5(294891) c6( ) c7(ck, ou, T, fM, oA, jh, bw, hn, gn, hF, bt, br); #c1(LYL1) c2(XP_011526318) c3(protein lyl-1 isoform X1) c4( ) c5(320606, 320607, 320608, 320609, 320610) c6( ) c7(ck, ou, T, fM, oA, jh, bw, hn, gn, hF, bt, br); #c1(LYN) c2(NP_001104567) c3(tyrosine-protein kinase Lyn isoform B) c4(42179) c5(42180, 42181, 42182, 42183, 42184, 42185) c6( ) c7(T, fM, bkF, jh, eA, q, dP, ib, aT, kH, h, blY, hn, ox, d, iU, aW, eZ, r, iV, ck, bt); #c1(LYN) c2(XP_011515833) c3(tyrosine-protein kinase Lyn isoform X1) c4(308794, 308795) c5(308796, 308797, 308798, 308799, 308800, 308801, 308802, 308803, 308804, 308805, 308806, 308807) c6( ) c7(T, fM, bkF, jh, eA, q, dP, ib, aT, kH, h, blY, hn, ox, d, iU, aW, eZ, r, iV, ck, bt); #c1(LYNX1) c2(NP_803253) c3(ly-6/neurotoxin-like protein 1 isoform b precursor) c4(252593) c5(252594, 252595, 252596, 252597, 252598, 252599) c6( ) c7(ci, aT, cr, Jr); #c1(LYNX1) c2(NP_803429) c3(ly-6/neurotoxin-like protein 1 isoform c precursor) c4(252600, 252601, 252602) c5(252603, 252604, 252605, 252606, 252607, 252608, 252609, 252610, 252611, 252612, 252613, 252614, 252615, 252616, 252617, 252618, 252619, 252620, 252621, 252622, 252623, 252624, 252625, 252626, 252627, 252628) c6( ) c7(ci, aT, cr, Jr); #c1(LYNX1) c2(XP_011515540) c3(ly-6/neurotoxin-like protein 1 isoform X1) c4(308367) c5(308368, 308369, 308370, 308371, 308372, 308373) c6( ) c7(ci, aT, cr, Jr); #c1(LYPD1) c2(NP_653187) c3(ly6/PLAUR domain-containing protein 1 isoform a precursor) c4(238978, 238979) c5(238980, 238981, 238982, 238983, 238984, 238985) c6( ) c7(rz); #c1(LYPD1) c2(XP_005263640) c3(ly6/PLAUR domain-containing protein 1 isoform X3) c4(278498) c5(278499, 278500, 278501, 278502, 278503, 278504) c6( ) c7(rz); #c1(LYPD1) c2(XP_005263641) c3(ly6/PLAUR domain-containing protein 1 isoform X1) c4( ) c5(278505, 278506, 278507, 278508, 278509) c6( ) c7(rz); #c1(LYPD2) c2(NP_991108) c3(ly6/PLAUR domain-containing protein 2 precursor) c4(263424) c5(263425, 263426, 263427, 263428, 263429, 263430) c6( ) c7(Ea); #c1(LYPD3) c2(NP_055215) c3(ly6/PLAUR domain-containing protein 3 precursor) c4(179883, (79884) c5(179885, 179886, 179887, 179888, 179889, 179890, 179891, 179892, 179893, 179894, 179895, 179896) c6( ) c7(av, T, Y, ff, r, aT, n, en, g, it, q); #c1(LYPD4) c2(NP_001278348) c3(ly6/PLAUR domain-containing protein 4 precursor) c4(109583) c5(109584, 109585, 109586, 109587, 109588, 109589) c6( ) c7(T, if, cbc, bB, j, qN, ad, aE, aT, dL, ae, eL, mu, aW, bw, t, bz, bP, bE, MW, wx, m); #c1(LYPD4) c2(NP_775777) c3(ly6/PLAUR domain-containing protein 4 precursor) c4(250259) c5(250260, 250261, 250262, 250263, 250264, 250265) c6( ) c7(T, if, cbc, bB, j, qN, ad, aE, aT, dL, ae, eL, mu, aW, bw, t, bz, bP, bE, MW, wx, m); #c1(LYPD5) c2(NP_001026919) c3(ly6/PLAUR domain-containing protein 5 isoform A precursor) c4(29989, 29990) c5(29991, 29992, 29993, 29994, 29995, 29996, 29997) c6( ) c7(T, fM, aE, eM, aW, Y, r, aT, aA, aF, ci, g, ae, q, ad, hX); #c1(LYPD5) c2(NP_872379) c3(ly6/PLAUR domain-containing protein 5 isoform B) c4( ) c5(256284, 256285, 256286, 256287, 256288) c6( ) c7(T, fM, aE, eM, aW, Y, r, aT, aA, af, ci, g, ae, q, ad, hX); #c1(LYPD6) c2(NP_001182614) c3(ly6/PLAUR domain-containing protein 6 precursor) c4(75115, 75116) c5(75117, 75118, 75119, 75120, 75121, 75122, 75123, 75124, 75125, 75126, 75127, 75128, 75129, 75130, 75131, 75132) c6( ) c7(cbd); #c1(LYPD8) c2(NP_001078943) c3(ly6/PLAUR domain-containing protein 8 preproprotein) c4(38148) c5(38149, 38150, 38151, 38152, 38153, 38154) c6( ) c7(ai, j); #c1(LYPLA1) c2(NP_001266286) c3(acyl-protein thioesterase 1 isoform 3) c4(99573) c5(99574, 99575, 99576, 99577, 99578, 99579) c6( ) c7(dD, ea, aj, eq, ey, mK, J); #c1(LYPLA1) c2(NP_006321) c3(acyl-protein thioesterase 1 isoform 1) c4(164019) c5(164020, 164021, 164022, 164023, 164024, 164025) c6( ) c7(dD, ea, aj, eq, ey, mk, J); #c1(LYPLAL1) c2(NP_001287699) c3(lysophospholipase-like protein 1 isoform c) c4(112575) c5(112576, 112577, 112578, 112579, 112580, 112581) c6( ) c7(aj, v, fc, cP, Be); #c1(LYPLAL1) c2(NP_620149) c3(lysophospholipase-like protein 1 isoform a) c4(237367) c5(237368, 237369, 237370, 237371, 237372, 237373) c6( ) c7(aj, v, fc, cP, Be); #c1(LYRM1) c2(NP_001289765) c3(LYR motif-containing protein 1) c4(114598) c5(114599, 114600, 114601, 114602, 114603, 114604, 114605, 114606, 114607, 114608, 114609, 114610, 114611, 114612, 114613, 114614, 114615, 114616) c6( ) c7(v); #c1(LYRM1) c2(XP_011544201) c3(LYR motif-containing protein 1 isoform X2) c4( ) c5(343341, 343342) c6( ) c7(v); #c1(LYRM4) c2(NP_001158312) c3(LYR motif-containing protein 4 isoform 2) c4(63599) c5(63600, 63601, 63602, 63603, 63604, 63605, 63606, 63607) c6( ) c7(oe, fe, K, cbe, iV, cm); #c1(LYRM4) c2(NP_001158313) c3(LYR motif-containing protein 4 isoform 3) c4(63608) c5(63609, 63610, 63611, 63612, 63613, 63614) c6( ) c7(de, fe, K, che, iV, cm); #c1(LYRM4) c2(NP_065141) c3(LYR motif-containing protein 4 isoform 1) c4(204248) c5(204249, 204250, 204251, 204252, 204253, 204254) c6( ) c7(oe, fe, K, cbe, iV, cm); #c1(LYRM7) c2(NP_859056) c3(complex III assembly factor LYRM7 isoform 1) c4(255427) c5(255428, 255429, 255430, 255431, 255432, 255433) c6( ) c7(hb); #c1(LYRM9) c2(NP_001070148) c3(LYR motif-containing protein 9) c4(34594) c5(34595, 34596, 34597, 34598, 34599, 34600, 34601, 34602, 34603) c6( ) c7(bM, ft); #c1(LYRM9) c2(XP_011522794) c3(LYR motif-containing protein 9 isoform X2) c4( ) c5(317303) c6( ) c7(bM, ft); #c1(LYST) c2(NP_001288294) c3(lysosomal-trafficking regulator) c4(113720) c5(113721, 113722, 113723, 113724, 113725, 113726, 113727, 113728, 113729, 113730, 113731, 113732, 113733, 113734, 113735, 113736) c6( ) c7(Nq, cT, Nr, lE, vD, rT, XC, U, lF, po, yH, pz, cbb, cd); #c1(LYST) c2(XP_011542339) c3(lysosomal-trafficking regulator isoform X5) c4(340913) c5( ) c6( ) c7(Nq, cT, Nr, lE, vD, rT, XC, U, lF, po, yH, pz, cbb, cd); #c1(LYVE1) c2(NP_006682) c3(lymphatic vessel endothelial hyaluronic acid receptor 1 precursor) c4(167121) c5(167122, 167123, 167124, 167125, 167126, 167127) c6( ) c7(T, iW, al, ci, q, ad, eA, Jm, bll, aT, Vv, PD, brC, JK, K, ae, aW, Y, hl, r, aq, iR, iK, bPK, gk, dB, B, vi); #c1(LYZ) c2(NP_000230) c3(lysozyme C precursor) c4(5266, 5267, 5268, 5269) c5(5270, 5271, 5272, 5273, 5274, 5275) c6( ) c7(kV, Kd, sY, dD, aXW, qb, ea, fN, aA, cz, jh, aj, BF, eq, ai, J, eA, alZ, h, bRd, cbf, K, dQ, H, fb, amO, af, Yl, ajV, s, ih, hl, fe, aq, aF, hp, g, iV, YZ, uC, zV, bF, ig, WP, dh, aQG, wR, bas, dy, R, y, bt, B); #c1(LYZL1) c2(NP_115906) c3(lysozyme-like protein 1) c4(224602) c5(224603, 224604, 224605, 224606, 224607, 224608, 224609, 224610) c6(224611, 224612) c7(aj, J); #c1(LYZL2) c2(NP_898881) c3(lysozyme-like protein 2) c4(257814, 257815) c5(257816, 257817, 257818, 257819, 257820, 257821) c6( ) c7(ki, W); #c1(LYZL6) c2(NP_001186880) c3(lysozyme-like protein 6 precursor) c4(78742) c5(78743, 78744, 78745, 78746, 78747, 78748, 78749, 78750, 78751, 78752, 78753, 78754, 78755, 78756) c6( ) c7(aw); #c1(LZTFL1) c2(NP_001263307) c3(leucine zipper transcription factor-like protein 1 isoform 2) c4(97147) c5(97148, 97149, 97150, 97151, 97152) c6( ) c7(T, aA, aF, zG, Tp, hH); #c1(LZTFL1) c2(NP_001263308) c3(leucine zipper transcription factor-like protein 1 isoform 3) c4(97153) c5(97154, 97155, 97156, 97157, 97158, 97159) c6( ) c7(T, aA, aF, zG, Tp, hH); #c1(LZTFL1) c2(NP_065080) c3(leucine zipper transcription factor-like protein 1 isoform 1) c4(203735) c5(203736, 203737, 203738, 203739, 203740, 203741, 203742) c6( ) c7(T, aA, aF, zG, Tp, hH); #c1(LZTR1) c2(NP_006758) c3(leucine-zipper-like transcriptional regulator 1) c4(167777, 167778, 167779) c5(167780, 167781, 167782, 167783, 167784, 167785, 167786, 167787, 167788, 167789, 167790, 167791, 167792, 167793, 167794, 167795, 167796, 167797) c6( ) c7(d, ac, cbg, Ls, oe, ayg); #c1(LZTS1) c2(XP_011542689) c3(leucine zipper putative tumor suppressor 1 isoform X1) c4(341227, 341228, 341229, 341230) c5(341231, 341232, 341233, 341234, 341235, 341236, 341237, 341238, 341239, 341240, 341241, 341242, 341243, 341244, 341245, 341246, 341247, 341248, 341249, 341250, 341251, 341252, 341253, 341254, 341255, 341256, 341257, 341258) c6( ) c7(Gl, T, id, oe, ht, aA, aj, hy, j, ci, ad, aT, Gw, dL, bK, ae, eL, aW, Y, AQ, ff, gl, aF, en, Tp, g, t, bz, iP, ac, m, Gu); #c1(M6PR) c2(NP_001193953) c3(cation-dependent mannose-6-phosphate receptor isoform 2 precursor) c4( ) c5(83317, 83318, 83319, 83320, 83321, 83322, 83323, 83324, 83325, 83326, 83327, 83328) c6( ) c7(fR, cz, aA, fr, af, ad); #c1(M6PR) c2(XP_011518974) c3(cation-dependent mannose-6-phosphate receptor isoform X1) c4(313428, 313429, 313430) c5(313431, 313432, 313433, 313434, 313435, 313436, 313437, 313438) c6( ) c7(fR, cz, aA, fr, af, ad); #c1(MAATS1) c2(NP_203528) c3(protein MAATS1) c4(228424, 228425, 228426) c5(228427, 228428, 228429, 228430, 228431, 228432) c6( ) c7(fR); #c1(MAATS1) c2(XP_011511579) c3(protein MAATS1 isoform X3) c4( ) c5(302309) c6( ) c7(fR); #c1(MAATS1) c2(XP_011511580) c3(protein MAATS1 isoform X4) c4( ) c5(302310, 302311, 302312, 302313, 302314, 302315, 302316, 302317, 302318, 302319, 302320) c6( ) c7(fR); #c1(MAB21L1) c2(NP_005575) c3(protein mab-21-like 1) c4(157585) c5(157586, 157587, 157588, 157589, 157590, 157591) c6( ) c7(aR); #c1(MAB21L2) c2(NP_006430) c3(protein mab-21-like 2) c4(165148) c5(165149, 165150, 165151, 165152, 165153, 165154) c6( ) c7(y, bA); #c1(MAB21L3) c2(XP_011538928) c3(protein mab-21-like 3 isoform X1) c4(336996) c5(336997, 336998, 336999, 337000, 337001, 337002) c6( ) c7(aw); #c1(MACC1) c2(NP_877439) c3(metastasis-associated in colon cancer protein 1) c4(256834) c5(256835, 256836, 256837, 256838, 256839, 256840, 256841, 256842, 256843, 256844) c6( ) c7(T, Dd, if, al, aA, bB, n, j, ci, qN, q, eA, iu, aT, io, hB, aW, eH, r, aF, VI, g, as, eF, t, gk, bE, m, B); #c1(MACROD1) c2(NP_054786) c3(O-acetyl-ADP-ribose deacetylase MACROD1) c4(177833) c5(177834, 177835, 177836, 177837, 177838, 177839) c6( ) c7(ck, aW, cl, hL, aT); #c1(MACROD2) c2(NP_001028259) c3(O-acetyl-ADP-ribose deacetylase MACROD2 isoform 2) c4(30430) c5(30431, 30432, 30433, 30434, 30435, 30436, 30437, 30438, 30439) c6( ) c7(c, pN, oe, aw, aA, bS, j, v, aF, an, bUh, lB, bN); #c1(MACROD2) c2(NP_542407) c3(O-acetyl-ADP-ribose deacetylase MACROD2 isoform 1) c4(232386, 232387) c5(232388, 232389, 232390, 232391, 232392, 232393, 232394, 232395, 232396, 232397, 232398, 232399, 232400) c6( ) c7(c, pN, De, aw, aA, bS, j, v, aF, an, bUh, lB, bN); #c1(MAD1L1) c2(NP_001291452) c3(mitotic spindle assembly checkpoint protein MAD1 isoform a) c4(116080) c5(116081, 116082, 116083, 116084, 116085, 116086, 116087, 116088, 116089, 116090, 116091, 116092, (16093, 116094) c6( ) c7(T, aw, jh, bB, fr, ci, hF, q, br, ad, cF, ft, aT, bK, he, pi, jf, d, B, s, aW, Y, oV, r, aq, aF, iR, t, cm, bz, Ld, bw, bE, m, bt, ne); #c1(MAD1L1) c2(NP_001291453) c3(mitotic spindle assembly checkpoint protein MAD1 isoform b precursor) c4(116095) c5(116096, 116097, 116098, 116099, 116100, 116101) c6( ) c7(T, aw, jh, bB, fr, ci, hF, q, br, ad, cF, ft, aT, bK, he, pi, jf, d, B, s, aW, Y, oV, r, aq, aF, iR, t, cm, bz, Ld, bw, bE, m, bt, ne); #c1(MAD1L1) c2(XP_011513869) c3(mitotic spindle assembly checkpoint protein MAD1 isoform X1) c4(306281) c5( ) c6( ) c7(T, aw, jh, bB, fr, ci, hF, q, br, ad, cF, ft, aT, bK, he, pi, jf, d, B, s, aW, Y, oV, r, aq, aF, iR, t, cm, bz, Ld, bw, bE, m, bt, ne); #c1(MAD1L1) c2(XP_011513872) c3(mitotic spindle assembly checkpoint protein MAD1 isoform X5) c4(306282) c5(306283, 306284, 306285, 306286, 306287, 306288) c6( ) c7(T, aw, jh, bB, fr, ci, hF, q, br, ad, cF, ft, aT, bK, he, pi, jf, d, B, s, aW, Y, oV, r, aq, aF, iR, t, cm, bz, Ld, bw, bE, m, bt, ne); #c1(MAD1L1) c2(XP_011513873) c3(mitotic spindle assembly checkpoint protein MAD1 isoform X4) c4( ) c5(306289, 306290, 306291, 306292, 306293, 306294) c6( ) c7(T, aw, jh, bB, fr, ci, hF, q, br, ad, cF, ft, aT, bK, he, pi, jf, d, B, s, aW, Y, oV, r, aq, aF, iR, t, cm, bz, Ld, bw, bE, m, bt, ne); #c1(MAD2L1) c2(NP_002349) c3(mitotic spindle assembly checkpoint protein MAD2A) c4(127713) c5(127714, 127715, 127716, 127717, 127718, 127719) c6( ) c7(T, id, EQ, aA, n, ci, q, av, no, py, Db, aT, dL, ey, eL, kl, aiB, qt, aW, ff, eH, LZ, r, aq, aF, en, g, eF, t, iV, bz, es, bP, eM, nB, bE, iA, m, it, B); #c1(MAD2L1BP) c2(NP_001003690) c3(MAD2L1-binding protein isoform 1) c4(19897) c5(19898, 19899, 19900, 19901, 19902, 19903) c6( ) c7(xF, bkY, n, fu, bX, UX, cbh, no, aV, QJ, kf, bMf, bU, aCg, bYA, bT, xz, bhi, bMj, dr, ql, bk, afO, B); #c1(MAD2L1BP) c2(NP_055443) c3(MAD2L1-binding protein isoform 2) c4(181166) c5(181167, 181168, 181169, 181170, 181171, 181172) c6( ) c7(xF, bkY, n, fu, bX, UX, cbh, no, aV, QJ, kf, bMf, bU, aCg, bYA, bT, xz, bhi, bMj, dr, ql, bk, afO, B); #c1(MAD2L2) c2(XP_011538809) c3(mitotic spindle assembly checkpoint protein MAD2B isoform X1) c4(336545, 336546, 336547, 336548, 336549, 336550) c5(336551, 336552, 336553, 336554, 336555, 336556, 336557, 336558, 336559, 336560, 336561, 336562, 336563, 336564, 336565, 336566, 336567, 336568, 336569, 336570, 336571, 336572, 336573, 336574, 336575, 336576, 336577, 336578, 336579, 336580, 336581, 336582, 336583, 336584, 336585, 336586, 336587, 336588, 336589, 336590, 336591, 336592, 336593, 336594, 336595, 336596, 336597, 336598) c6( ) c7(T, bE, aW, r, aT, bB, BF, m, fr, lu, f, q); #c1(MADCAM1) c2(NP_570116) c3(mucosal addressin cell adhesion molecule 1 isoform a precursor) c4(233941, 233942, 233943, 233944) c5(233945, 233946, 233947, 233948, 233949, 233950, 233951, 233952, 233953, 233954, 233955, 233956) c6( ) c7(A, iM, je, aD, kH, hN, cbi, ji, iV, XI); #c1(MADCAM1) c2(NP_570118) c3(mucosal addressin cell adhesion molecule 1 isoform b precursor) c4(233957, 233958) c5(233959, 233960, 233961, 233962, 233963, 233964, 233965, 233966, 233967, 233968, 233969, 233970, 233971, 233972, 233973, 233974, 233975, 233976, 233977, 233978, 233979, 233980, 233981, 233982) c6( ) c7(A, iM, je, aD, kH, hN, cbi, ji, iV, XI); #c1(MADD) c2(NP_569831) c3(MAP kinase-activating death domain protein isoform g) c4(233927) c5(233928, 233929, 233930, 233931, 233932, 233933) c6( ) c7(bL, qJ, T, hd, nB, cj, wl, By, aj, hp, j, vs, cm, af, J); #c1(MADD) c2(NP_569832) c3(MAP kinase-activating death domain protein isoform h) c4(233934) c5(233935, 233936, 233937, 233938, 233939, 233940) c6( ) c7(bL, qJ, T, hd, nB, cj, wl, By, aj, hp, j, vs, cm, af, J); #c1(MADD) c2(XP_005253246) c3(MAP kinase-activating death domain protein isoform X6) c4(272296) c5(272297, 272298, 272299, 272300, 272301, 272302) c6( ) c7(bL, qJ, T, hd, nB, cj, wl, By, aj, hp, j, vs, cm, af, J); #c1(MADD) c2(XP_005253253) c3(MAP kinase-activating death domain protein isoform X4) c4(272303) c5(272304, 272305, 272306, 272307, 272308, 272309) c6( ) c7(bL, qJ, T, hd, nB, cj, wl, By, aj, hp, j, vs, cm, af, J); #c1(MADD) c2(XP_005253256) c3(MAP kinase-activating death domain protein isoform X1) c4(272310) c5(272311, 272312, 272313, 272314, 272315, 272316) c6( ) c7(bL, qJ, T, hd, nB, cj, wl, By, aj, hp, j, vs, cm, af, J); #c1(MADD) c2(XP_005253258) c3(MAP kinase-activating death domain protein isoform X2) c4(272317) c5(272318, 272319, 272320, 272321, 272322, 272323) c6( ) c7(bL, qJ, T, hd, nB, cj, wl, By, aj, hp, j, vs, cm, af, J); #c1(MADD) c2(XP_005253260) c3(MAP kinase-activating death domain protein isoform X3) c4(272324) c5(272325, 272326, 272327, 272328, 272329, 272330) c6( ) c7(bL, qJ, T, hd, nB, cj, wl, By, aj, hp, j, vs, cm, af, J); #c1(MADD) c2(XP_005253261) c3(MAP kinase-activating death domain protein isoform X7) c4(272331) c5(272332, 272333, 272334, 272335, 272336, 272337) c6( ) c7(bL, qJ, T, hd, nB, cj, wl, By, aj, hp, j, vs, cm, af, J); #c1(MADD) c2(XP_005253262) c3(MAP kinase-activating death domain protein isoform X5) c4(272338, 272339) c5(272340, 272341, 272342, 272343, 272344, 272345, 272346, 272347, 272348, 272349, 272350, 272351) c6( ) c7(bL, qJ, T, hd, nB, cj, wl, By, aj, hp, j, vs, cm, af, J); #c1(MAEA) c2(NP_001017405) c3(macrophage erythroblast attacher isoform 1) c4(26440, 26441, 26442, 26443) c5(26444, 26445, 26446, 26447, 26448, 26449, 26450, 26451, 26452, 26453, 26454, 26455, 26456, 26457, 26458, 26459, 26460, 26461) c6( ) c7(bL, ea, dD, eq); #c1(MAEA) c2(NP_001284359) c3(macrophage erythroblast attacher isoform 3) c4(111771) c5(111772, 111773, 111774, 111775, 111776, 111777) c6( ) c7(bL, ea, dD, eq); #c1(MAEA) c2(NP_001284360) c3(macrophage erythroblast attacher isoform 4) c4(111778, 111779) c5(111780, 111781, 111782, 111783, 111784, 111785, 111786, 111787, 111788, 111789, 111790, 111791) c6( ) c7(bL, ea, dD, eq); #c1(MAEA) c2(NP_001284361) c3(macrophage erythroblast attacher isoform 5) c4(111792) c5(111793, 111794, 111795, 111796, 111797, 111798, 111799) c6( ) c7(bL, ea, dD, eq); #c1(MAEA) c2(NP_001284362) c3(macrophage erythroblast attacher isoform 6) c4( ) c5(111800, 111801, 111802, 111803, 111804) c6( ) c7(bL, ea, dD, eq); #c1(MAEA) c2(NP_005873) c3(macrophage erythroblast attacher isoform 2) c4(160060) c5(160061, 160062, 160063, 160064, 160065, 160066) c6( ) c7(bL, ea, dD, eq); #c1(MAEL) c2(NP_001273306) c3(protein maelstrom homolog isoform 2) c4(104649, 104650) c5(104651, 104652, 104653, 104654, 104655, 104656, 104657, 104658, 104659, 104660, 104661, 104662) c6( ) c7(cz, r, m, t, q, bA); #c1(MAEL) c2(NP_116247) c3(protein maelstrom homolog isoform 1) c4(225949) c5(225950, 225951, 225952, 225953, 225954, 225955) c6( ) c7(cz, r, m, t, q, bA); #c1(MAEL) c2(XP_006711646) c3(protein maelstrom homolog isoform X3) c4(286184) c5( ) c6( ) c7(cz, r, m, t, q, bA); #c1(MAEL) c2(XP_011508370) c3(protein maelstrom homolog isoform X4) c4( ) c5(299063, 299064, 299065, 299066, 299067, 299068) c6( ) c7(cz, r, m, t, q, bA); #c1(MAF) c2(NP_001026974) c3(transcription factor Maf isoform b) c4( ) c5(30019, 30020, 30021, 30022, 30023, 30024) c6( ) c7(bL, T, dD, ea, aw, bO, fd, v, j, ah, eq, aSL, jw, Vp, XY, hP, qM, bVH, bN, kn, cbj, amS, W, aor); #c1(MAF) c2(NP_005351) c3(transcription factor Maf isoform a) c4(155331) c5(155332, 155333, 155334, 155335, 155336, 155337, 155338) c6( ) c7(bL, T, dD, ea, aw, bO, fd, v, j, ah, eq, aSL, jw, Vp, XY, hP, qM, bVH, bN, kn, cbj, amS, W, aor); #c1(MAF) c2(XP_011521386) c3(transcription factor Maf isoform X1) c4(316195) c5( ) c6( ) c7(bL, T, dD, ea, aw, bO, fd, v, j, ah, eq, aSL, jw, Vp, XY, hP, qM, bVH, bN, kn, cbj, amS, W, aor); #c1(MAF1) c2(NP_115648) c3(repressor of RNA polymerase III transcription MAF1 homolog) c4(223429, 223430) c5(223431, 223432, 223433, 223434, 223435, 223436, 223437, 223438, 223439, 223440, 223441, 223442, 223443, 223444, 223445, 223446) c6( ) c7(d, K); #c1(MAFA) c2(NP_963883) c3(transcription factor MafA) c4(262488) c5(262489, 262490, 262491, 262492, 262493, 262494, 262495) c6( ) c7(bL, A, aj, dG, J, yF); #c1(MAFB) c2(NP_005452) c3(transcription factor MafB) c4(156299) c5(156300, 156301, 156302, 156303, 156304, 156305, 156306, 156307, 156308, 156309, 156310) c6( ) c7(bL, Na, aC, cbk, amS, nX, jh, fd, Nc, y, fq); #c1(MAFF) c2(NP_001155045) c3(transcription factor MafF isoform a) c4(61324, 61325, 61326, 61327) c5(61328, 61329, 61330, 61331, 61332, 61333, 61334, 61335, 61336, 61337, 61338, 61339, 61340, 61341, 61342, 61343, 61344, 61345, 61346, 61347, 61348, 61349, 61350, 61351, 61352, 61353, 61354) c6( ) c7(Y); #c1(MAFF) c2(NP_001155046) c3(transcription factor MafF isoform b) c4( ) c5(61355, 61356, 61357, 61358, 61359, 61360, 61361, 61362, 61363, 61364) c6( ) c7(Y); #c1(MAFG) c2(XP_011521881) c3(transcription factor MafG isoform X1) c4(316480) c5(316481, 316482, 316483, 316484, 316485, 316486, 316487, 316488, 316489, 316490, 316491) c6( ) c7(bz, jw, r); #c1(MAFK) c2(XP_006715836) c3(transcription factor MafK isoform X1) c4(289867) c5(289868, 289869, 289870, 289871, 289872, 289873, 289874, 289875, 289876, 289877, 289878, 289879, 289880, 289881) c6( ) c7(ck, eM, Y, iS, fd, bw, v, K, hF, vt, B, ip); #c1(MAG) c2(NP_001186145) c3(myelin-associated glycoprotein isoform c) c4(77218) c5(77219, 77220, 77221, 77222, 77223) c6( ) c7(ak, aC, wi, akQ, ac, ob, fm, cbl, My, an, fu, R, bU, cb, cbm, V, bz, eA, dr); #c1(MAG) c2(NP_002352) c3(myelin-associated glycoprotein isoform a precursor) c4(127720, 127721) c5(127722, 127723, 127724, 127725, 127726, 127727, 127728, 127729, 127730, 127731, 127732, 127733, 127734, 127735) c6( ) c7(ak, aC, wi, akQ, ac, ob, fm, cbl, My, an, fu, R, bU, cb, cbm, V, bz, eA, dr); #c1(MAG) c2(NP_542167) c3(myelin-associated glycoprotein isoform b precursor) c4(232158) c5(232159, 232160, 232161, 232162, 232163) c6( ) c7(ak, aC, wi, akQ, ac, ob, fm, cbl, My, an, fu, R, bU, cb, cbm, V, bz, eA, dr); #c1(MAGEA1) c2(NP_004979) c3(melanoma-associated antigen 1) c4(151570) c5(151571, 151572, 151573, 151574, 151575, 151576) c6( ) c7(nZ, G, T, id, wu, aoE, aA, fd, n, ci, ad, av, avA, Nn, kH, aT, wm, xQ, d, aW, Y, ff, aF, en, bu, t, cm, iK, ck, Dy, eM, cj, m, B); #c1(MAGEA10) c2(NP_001011543) c3(melanoma-associated antigen 10) c4(24516, 24517, 24518) c5(24519, 24520, 24521, 24522, 24523, 24524, 24525, 24526, 24527, 24528, 24529, 24530, 24531, 24532, 24533, 24534, 24535, 24536, 24537, 24538, 24539, 24540) c6( ) c7(Y); #c1(MAGEA11) c2(NP_001011544) c3(melanoma-associated antigen 11 isoform b) c4(24541) c5(24542, 24543, 24544, 24545, 24546, 24547, 24548, 24549, 24550) c6( ) c7(T, ae, ad); #c1(MAGEA11) c2(NP_005357) c3(melanoma-associated antigen Il isoform a) c4(155418) c5(155419, 155420, 155421, 155422, 155423, 155424) c6( ) c7(T, ae, ad); #c1(MAGEA11) c2(XP_011529466) c3(melanoma-associated antigen Il isoform X2) c4( ) c5(324890) c6( ) c7(T, ae, ad); #c1(MAGEA12) c2(NP_001159858) c3(melanoma-associated antigen (2) c4(64848) c5(64849, 64850, 64851, 64852, 64853, 64854, 64855, 64856, 64857, 64858, 64859, 64860, 64861, 64862) c6( ) c7(Y, ff, en); #c1(MAGEA2B) c2(NP_705692) c3(melanoma-associated antigen 2) c4(246203, 246204, 246205, 246206, 246207, 246208) c5(246209, 246210, 246211, 246212, 246213, 246214, 246215, 246216, 246217, 246218, 246219, 246220, 246221, 246222, 246223, 246224, 246225, 246226, 246227, 246228, 246229, 246230, 246231, 246232, 246233, 246234, 246235, 246236, 246237, 246238, 246239, 246240, 246241, 246242, 246243, 246244, 246245, 246246, 246247, 246248, 246249, 246250, 246251, 246252, 246253, 246254, 246255, 246256, 246257, 246258, 246259, 246260, 246261, 246262, 246263, 246264, 246265, 246266, 246267) c6( ) c7(Dy, Y, AJ, bX, bu, bz, eA, iP); #c1(MAGEA3) c2(NP_005353) c3(melanoma-associated antigen 3) c4(155339, 155340) c5(155341, 155342, 155343, 155344, 155345, 155346, 155347, 155348, 155349, 155350, 155351, 155352, 155353, 155354, 155355, 155356) c6( ) c7(nZ, G, T, aC, wu, ht, aA, n, ci, aHy, ad, hX, av, ft, aE, aT, dL, wm, hu, ae, eL, cp, xQ, aW, Y, ff, hl, lo, kp, af, en, bu, t, aoG, bz, cm, iK, ck, gX, m, B, ao); #c1(MAGEA4) c2(NP_001011549) c3(melanoma-associated antigen 4) c4(24551, 24552, 24553, 24554, 24555) c5(24556, 24557, 24558, 24559, 24560, 24561, 24562, 24563, 24564, 24565, 24566, 24567, 24568, 24569, 24570, 24571, 24572, 24573, 24574, 24575, 24576, 24577, 24578, 24579, 24580, 24581, 24582, 24583, 24584, 24585, 24586, 24587, 24588, 24589, 24590, 24591, 24592, 24593, 24594, 24595, 24596, 24597, 24598, 24599, 24600, 24601, 24602, 24603, 24604, 24605, 24606, 24607, 24608, 24609, 24610) c6( ) c7(av, G, id, eM, Y, ff, Qm, ci, en, bu, g, bz, B, ad); #c1(MAGEA4) c2(XP_005274734) c3(melanoma-associated antigen 4 isoform X2) c4( ) c5(285352, 285353, 285354, 285355, 285356, 285357, 285358, 285359, 285360, 285361, 285362) c6( ) c7(av, G, id, eM, Y, ff, Qm, ci, en, bu, g, bz, B, ad); #c1(MAGEA6) c2(NP_005354) c3(melanoma-associated antigen 6) c4(155357, 155358, 155359, 155360) c5(155361, 155362, 155363, 155364, 155365, 155366, 155367, 155368, 155369, 155370, 155371, 155372, 155373, 155374, 155375, 155376, 155377, 155378) c6( ) c7(ds, ff, fr, Y, en); #c1(MAGEA9) c2(NP_005356) c3(melanoma-associated antigen 9) c4(155379, 155380, 155381, 155382, 155383) c5(155384, 155385, 155386, 155387, 155388, 155389, 155390, 155391, 155392, 155393, 155394, 155395, 155396, 155397, 155398, 155399, 155400, 155401, 155402, 155403, 155404, 155405, 155406, 155407, 155408, 155409, 155410, 155411, 155412, 155413, 155414, 155415, 155416, 155417) c6( ) c7(dL, fr); #c1(MAGEB1) c2(NP_803134) c3(melanoma-associated antigen B1) c4(252502) c5(252503, 252504, 252505, 252506, 252507, 252508, 252509, 252510, 252511, 252512, 252513, 252514, 252515, 252516) c6( ) c7(Y); #c1(MAGEB17) c2(NP_001264236) c3(melanoma-associated antigen B17) c4(97622, 97623) c5(97624, 97625, 97626, 97627, 97628, 97629, 97630, 97631, 97632, 97633, 97634, 97635, 97636, 97637, 97638, 97639) c6( ) c7(Hr); #c1(MAGEB2) c2(NP_002355) c3(melanoma-associated antigen B2) c4(127736) c5(127737, 127738, 127739, 127740, 127741, 127742) c6( ) c7(Y, fr, iK, eZ); #c1(MAGEB6) c2(NP_775794) c3(melanoma-associated antigen B6) c4(250334) c5(250335, 250336, 250337, 250338, 250339, 250340) c6( ) c7(pf, ff, NT, aA, aF, en); #c1(MAGEC1) c2(NP_005453) c3(melanoma-associated antigen CI) c4(156311, 156312, 156313, 156314) c5(156315, 156316, 156317, 156318, 156319, 156320, 156321, 156322, 156323, 156324, 156325, 156326, 156327, 156328, 156329, 156330, 156331, 156332, 156333, 156334, 156335, 156336, 156337, 156338) c6( ) c7(ae, T, baa, eM, Y, ff, aT, fd, en, bu, bz, ad, cp); #c1(MAGEC2) c2(NP_057333) c3(melanoma-associated antigen C2) c4(190171) c5(190172, 190173, 190174, 190175, 190176, 190177) c6( ) c7(T, iV, wu, aW, Y, oe, e, ff, aT, r, ci, q, bu, en, g, wm, bz, B, Hr, cp); #c1(MAGEC3) c2(NP_619647) c3(melanoma-associated antigen C3 isoform 1) c4(237007, 237008) c5(237009, 237010, 237011, 237012, 237013, 237014, 237015, 237016, 237017, 237018, 237019, 237020) c6( ) c7(Ej, T, zs); #c1(MAGEC3) c2(XP_011529569) c3(melanoma-associated antigen C3 isoform X1) c4(325122, 325123) c5(325124, 325125, 325126, 325127, 325128, 325129, 325130, 325131, 325132, 325133, 325134, 325135, 325136, 325137, 325138, 325139, 325140) c6( ) c7(Ej, T, zs); #c1(MAGED1) c2(NP_001005332) c3(melanoma-associated antigen D1 isoform b) c4(21352, 21353) c5(21354, 21355, 21356, 21357, 21358, 21359, 21360, 21361, 21362, 21363, 21364, 21365, 21366, 21367, 21368, 21369, 21370, 21371, 21372, 21373, 21374, 21375, 21376, 21377, 21378, 21379) c6( ) c7(T, hq, aC, aE, dB, Y, bS, v, ci, ct, eA, Z, rc, ad, bN, bdO); #c1(MAGED1) c2(NP_001005333) c3(melanoma-associated antigen D1 isoform a) c4(21380) c5(21381, 21382, 21383, 21384, 21385, 21386, 21387) c6( ) c7(T, hq, aC, aE, dB, Y, bS, v, ci, ct, eA, Z, rc, ad, bN, bdO); #c1(MAGED2) c2(NP_055414) c3(melanoma-associated antigen D2) c4(180926, 180927, 180928, 180929) c5(180930, 180931, 180932, 180933, 180934, 180935, 180936, 180937, 180938, 180939, 180940, 180941, 180942, 180943, 180944, 180945, 180946, 180947, 180948, 180949, 180950, 180951, 180952, 180953, 180954, 180955, 180956, 180957, 180958, 180959, 180960, 180961, 180962, 180963, 180964, 180965, 180966, 180967, 180968, 180969, 180970, 180971, 180972, 180973) c6( ) c7(cbn, aW, Y, aT, aA, ci, m, t); #c1(MAGED4) c2(NP_001092270) c3(melanoma-associated antigen D4 isoform 2) c4(39514) c5( ) c6( ) c7(nZ, T, id, aoE, fd, n, fr, ci, eA, av, avA, Nn, kH, aT, xQ, d, aW, Y, aF, bu, t, cm, iK, ck, Dy, eM, cj, m, B); #c1(MAGED4) c2(NP_001258990) c3(melanoma-associated antigen D4 isoform 3) c4(96901) c5(96902, 96903, 96904, 96905, 96906, 96907, 96908, 96909, 96910, 96911, 96912, 96913) c6( ) c7(nZ, T, id, aoE, fd, n, fr, ci, eA, av, avA, Nn, kH, aT, xQ, d, aW, Y, aF, bu, t, cm, iK, ck, Dy, eM, cj, m, B); #c1(MAGED4B) c2(NP_110428) c3(melanoma-associated antigen D4 isoform 1) c4(220298) c5(220299, 220300, 220301, 220302, 220303, 220304, 220305, 220306, 220307, 220308, 220309, 220310, 220311, 220312, 220313, 220314, 220315, 220316, 220317, 220318, 220319, 220320, 220321, 220322, 220323, 220324, 220325) c6( ) c7(nZ, T, id, aoE, fd, n, fr, ci, eA, av, avA, Nn, kH, aT, xQ, d, aW, Y, aF, bu, t, cm, iK, ck, Dy, eM, cj, m, B); #c1(MAGEE1) c2(NP_065983) c3(melanoma-associated antigen E1) c4(206598) c5(206599, 206600, 206601, 206602, 206603, 206604) c6( ) c7(ng, bL, T, dD, ea, zs, e, aA, cz, j, eq, aE, aT, cy, dL, K, eL, hm, A, d, aW, Y, aF, dw, ac, zr, aBr, mx, pP, y); #c1(MAGEH1) c2(NP_054780) c3(melanoma-associated antigen H1) c4(177783) c5(177784, 177785, 177786, 177787, 177788, 177789) c6( ) c7(aE, Y, eX, h, eV, K); #c1(MAGEL2) c2(NP_061939) c3(MAGE-like protein 2) c4(201223) c5(201224, 201225, 201226, 201227, 201228, 201229, 201230, 201231) c6( ) c7(cA, rz, Y, amj, v, cbo, Tp, he, rc, bN); #c1(MAGI1) c2(NP_001028229) c3(membrane-associated guanylate kinase, WW and PDZ domain-containing protein 1 isoform c) c4(30407) c5(30408, 30409, 30410, 30411, 30412, 30413) c6( ) c7(T, dA, n, fr, anu, ip, jz, ib, py, qK, Nn, aT, Ne, bK, K, anw, je, anv, nO, aW, Y, t, bw, bz, iP, sk, es, iM, Dy, iA, Ew, hk, m, anx, B, VB, hS); #c1(MAGI1) c2(NP_004733) c3(membrane-associated guanylate kinase, WW and PDZ domain-containing protein 1 isoform b) c4(149165) c5(149166, 149167, 149168, 149169, 149170, 149171) c6( ) c7(T, dA, n, fr, anu, ip, jz, ib, py, qK, Nn, aT, Ne, bK, K, anw, je, anv, nO, aW, Y, t, bw, bz, iP, sk, es, iM, Dy, iA, Ew, hk, m, anx, B, VB, hS); #c1(MAGI1) c2(NP_056335) c3(membrane-associated guanylate kinase, WW and PDZ domain-containing protein 1 isoform a) c4(187137, 187138) c5(187139, 187140, 187141, 187142, 187143, 187144, 187145, 187146, 187147, 187148, 187149, 187150) c6( ) c7(T, dA, n, fr, anu, ip, jz, ib, py, qK, Nn, aT, Ne, bK, K, anw, je, anv, nO, aW, Y, t, bw, bz, iP, sk, es, iM, Dy, iA, Ew, hk, m, anx, B, VB, hS); #c1(MAGI2) c2(NP_001288057) c3(membrane-associated guanylate kinase, WW and PDZ domain-containing protein 2 isoform 2) c4(113450, 113451) c5(113452, 113453, 113454, 113455, 113456, 113457) c6( ) c7(ea, Y, ha, qK, h, fe, nw, hp, wb, iV, f, bz, iu, cC); #c1(MAGI2) c2(NP_036433) c3(membrane-associated guanylate kinase, WW and PDZ domain-containing protein 2 isoform 1) c4(173822, 173823) c5(173824, 173825, 173826, 173827, 173828, 173829, 173830, 173831, 173832, 173833, 173834, 173835, 173836, 173837, 173838, 173839, 173840, (73841) c6( ) c7(ea, Y, ha, qK, h, fe, nw, hp, wb, iV, f, bz, iu, cC); #c1(MAGI2) c2(XP_011515020) c3(membrane-associated guanylate kinase, WW and PDZ domain-containing protein 2 isoform X1) c4(307800) c5(307801) c6( ) c7(ea, Y, ha, qK, h, fe, nw, hp, wb, iV, f, bz, iu, cC); #c1(MAGI2) c2(XP_011515027) c3(membrane-associated guanylate kinase, WW and PDZ domain-containing protein 2 isoform X6) c4( ) c5(307802, 307803, 307804, 307805, 307806, 307807) c6( ) c7(ea, Y, ha, qK, h, fe, nw, hp, wb, iV, f, bz, iu, cC); #c1(MAGI3) c2(XP_011539510) c3(membrane-associated guanylate kinase, WW and PDZ domain-containing protein 3 isoform X2) c4(337714) c5(337715, 337716, 337717, 337718, 337719, 337720, 337721) c6( ) c7(bE, hr, bB, hD, g, gT, X); #c1(MAGT1) c2(NP_115497) c3(magnesium transporter protein 1) c4(222916, 222917) c5(222918, 222919, 222920, 222921, 222922, 222923, 222924, 222925, 222926, 222927, 222928, 222929) c6( ) c7(T, if, eX, jh, fd, alH, ci, nA, PJ, qN, ai, ad, cbq, aE, dv, aT, kf, vH, eV, K, cbp, ae, cp, d, MD, nO, aW, Y, hl, aq, iR, bw, bz, LB, ck, kP, iT, bE, HN, bt, zA, B); #c1(MAK) c2(NP_005897) c3(serine/threonine-protein kinase MAK isoform 1) c4(160195, 160196, 160197) c5(160198, 160199, 160200, 160201, 160202, 160203, 160204, 160205, 160206, 160207, 160208) c6( ) c7(IR, cbr, nw, nC, ae, ad, de); #c1(MAK) c2(XP_011512924) c3(serine/threonine-protein kinase MAK isoform X1) c4( ) c5(304321, 304322, 304323, 304324, 304325, 304326, 304327, 304328, 304329, 304330, 304331) c6( ) c7(IR, cbr, nw, nC, ae, ad, de); #c1(MAK16) c2(NP_115898) c3(protein MAK16 homolog) c4(224571) c5(224572, 224573, 224574, 224575, 224576, 224577) c6( ) c7(bM, T, aC, ha, jh, aA, bB, tc, TM, Vd, ci, nA, lR, fr, hF, eA, aR, ad, hX, eW, ft, aE, WY, ds, SV, aT, kf, Ak, dV, h, K, an, brV, fb, ae, af, dx, iL, d, vT, aW, Y, ff, eZ, nf, aq, aaE, aF, Ku, en, iR, bz, pb, QA, bN, dR, ck, rR, kP, c, eM, bE, Qu, dh, mx, U, aEs, rx, xh, arg, B); #c1(MAL) c2(NP_002362) c3(myelin and lymphocyte protein isoform a) c4(127743) c5(127744, 127745, 127746, 127747, 127748, 127749) c6( ) c7(T, aHV, if, ht, eX, aA, hy, v, ci, qN, ad, aE, akU, dV, aT, h, eV, iY, hu, lZ, s, aW, ff, aq, af, en, f, t, iK, ck, gX, m, y, bt, B); #c1(MAL) c2(NP_071883) c3(myelin and lymphocyte protein isoform b) c4(212063) c5(212064, 212065, 212066, 212067, 212068, 212069) c6( ) c7(T, aHV, if, ht, eX, aA, hy, v, ci, qN, ad, aE, akU, dV, aT, h, eV, iY, hu, lZ, s, aW, ff, aq, aF, en, f, t, iK, ck, gX, m, y, bt, B); #c1(MAL) c2(NP_071884) c3(myelin and lymphocyte protein isoform c) c4(212070) c5(212071, 212072, 212073, 212074, 212075, 212076) c6( ) c7(T, aHV, if, ht, eX, aA, hy, v, ci, qN, ad, aE, akU, dV, aT, h, eV, iY, hu, lZ, s, aW, ff, aq, aF, en, f, t, iK, ck, gX, m, y, bt, B); #c1(MAL) c2(NP_071885) c3(myelin and lymphocyte protein isoform d) c4(212077) c5(212078, 212079, 212080, 212081, 212082, 212083) c6( ) c7(T, aHV, if, ht, eX, aA, hy, v, ci, qN, ad, aE, akU, dV, aT, h, eV, iY, hu, lZ, s, aW, ff, aq, aF, en, f, t, iK, ck, gX, m, y, bt, B); #c1(MALL) c2(NP_005425) c3(MAL-like protein) c4(156002) c5(156003, 156004, 156005, 156006, 156007, 156008) c6( ) c7(bB, bE, s); #c1(MALRD1) c2(NP_001135780) c3(MAM and LDL-receptor class A domain-containing protein 1 precursor) c4(53246, 53247, 53248) c5( ) c6( ) c7(Kc, mR, af, cz, aj, v, cd, sj); #c1(MALRD1) c2(XP_011517756) c3(MAM and LDL-receptor class A domain-containing protein 1 isoform X2) c4(311598, 311599) c5( ) c6( ) c7(Kc, mR, af, cz, aj, v, cd, sj); #c1(MALRD1) c2(XP_011517757) c3(MAM and LDL-receptor class A domain-containing protein 1 isoform X3) c4( ) c5(311600, 311601, 311602, 311603, 311604, 311605, 311606, 311607, 311608, 311609, 311610, 311611, 311612, 311613, 311614, 311615, 311616, 311617, 311618, 311619, 311620, 311621, 311622, 311623, 311624, 311625, 311626, 311627, 311628, 311629) c6( ) c7(Kc, mR, af, cz, aj, v, cd, sj); #c1(MALT1) c2(NP_006776) c3(mucosa-associated lymphoid tissue lymphoma translocation protein 1 isoform a) c4(167972) c5(167973, 167974, 167975, 167976, 167977, 167978) c6( ) c7(T, fM, aD, jh, asa, aA, fd, ci, V, cbs, apS, aJA, Df, uQ, oS, az, Of, aq, Og, arZ, asc, fT, SZ, ij); #c1(MALT1) c2(NP_776216) c3(mucosa-associated lymphoid tissue lymphoma translocation protein 1 isoform b) c4(251016) c5(251017, 251018, 251019, 251020, 251021, 251022) c6( ) c7(T, fM, aD, jh, asa, aA, fd, ci, V, cbs, apS, aJA, Df, uQ, oS, az, Of, aq, Og, arZ, asc, fT, SZ, ij); #c1(MAML1) c2(NP_055572) c3(mastermind-like protein 1) c4(182008, 182009) c5(182010, 182011, 182012, 182013, 182014, 182015, 182016, 182017, 182018, 182019, 182020, 182021, 182022) c6( ) c7(es, py, iA, if, fr, qN, it); #c1(MAML2) c2(NP_115803) c3(mastermind-like protein 2) c4(223961) c5(223962, 223963, 223964, 223965, 223966, 223967, 223968, 223969, 223970) c6( ) c7(T, aWj, ahG, cbt, iB, aWi, cbu, aw, aeX, ff, cj, jh, n, k, en, Tj, bw, bt, br, afb); #c1(MAML2) c2(XP_011541327) c3(mastermind-like protein 2 isoform X3) c4( ) c5(339923, 339924) c6( ) c7(T, aWj, ahG, cbt, iB, aWi, cbu, aw, aeX, ff, cj, jh, n, k, en, Tj, bw, bt, br, afb); #c1(MAML3) c2(NP_061187) c3(mastermind-like protein 3) c4(199851, 199852, 199853, 199854, 199855) c5(199856, 199857, 199858, 199859, 199860, 199861, 199862, 199863, 199864, 199865, 199866, 199867, 199868, 199869, 199870, 199871, 199872, 199873, 199874, 199875, 199876, 199877, 199878, 199879, 199880, 199881, 199882, 199883, 199884) c6( ) c7(bD, qK); #c1(MAMLD1) c2(NP_001170936) c3(mastermind-like domain-containing protein 1 isoform 1) c4(69598) c5(69599, 69600, 69601, 69602, 69603, 69604, 69605) c6( ) c7(cbv, adY, xz, cbw, adW, Pp, Ns, UX); #c1(MAMLD1) c2(NP_001170937) c3(mastermind-like domain-containing protein 1 isoform 3) c4(69606) c5(69607, 69608, 69609, 69610, 69611, 69612, 69613) c6( ) c7(cbv, adY, xz, cbw, adW, Pp, Ns, UX); #c1(MAMLD1) c2(NP_005482) c3(mastermind-like domain-containing protein 1 isoform 2) c4(156588, 156589) c5(156590, 156591, 156592, 156593, 156594, 156595, 156596, 156597, 156598, 156599, 156600, 156601, 156602, 156603, 156604, 156605, 156606) c6( ) c7(cbv, adY, xz, cbw, adW, Pp, Ns, UX); #c1(MAMLD1) c2(XP_011529395) c3(mastermind-like domain-containing protein 1 isoform X4) c4(324771) c5( ) c6( ) c7(cbv, adY, xz, cbw, adW, Pp, Ns, UX); #c1(MAMLD1) c2(XP_011529397) c3(mastermind-like domain-containing protein 1 isoform X5) c4( ) c5(324772, 324773, 324774, 324775, 324776, 324777) c6( ) c7(cbv, adY, xz, cbw, adW, Pp, Ns, UX); #c1(MAMLD1) c2(XP_011529398) c3(mastermind-like domain-containing protein 1 isoform X6) c4(324778) c5(324779, 324780, 324781, 324782, 324783, 324784) c6( ) c7(cbv, adY, xz, cbw, adW, Pp, Ns, UX); #c1(MAN1A1) c2(NP_005898) c3(mannosyl-oligosaccharide 1,2-alpha-mannosidase (A) c4(160209) c5( ) c6( ) c7(bL, ey, dv, h); #c1(MAN1A1) c2(XP_005267043) c3(mannosyl-oligosaccharide 1,2-alpha-mannosidase IA isoform X1) c4( ) c5(280632, 280633, 280634, 280635, 280636, 280637) c6( ) c7(bL, ey, dv, h); #c1(MAN1A1) c2(XP_011534136) c3(mannosyl-oligosaccharide 1,2-alpha-mannosidase IA isoform X3) c4(330410) c5(330411, 330412, 330413, 330414, 330415, 330416) c6( ) c7(bL, ey, dv, h); #c1(MAN1B1) c2(NP_057303) c3(endoplasmic reticulum mannosyl-oligosaccharide 1,2-alpha-mannosidase) c4(190003, 190004) c5(190005, 190006, 190007, 190008, 190009, 190010, 190011, 190012, 190013, 190014, 190015, 190016) c6( ) c7(T, am, SP, aq, K, nA, cbx, B, aDj); #c1(MAN1B1) c2(XP_006717008) c3(endoplasmic reticulum mannosyl-oligosaccharide 1,2-alpha-mannosidase isoform X1) c4(290650) c5(290651, 290652, 290653, 290654, 290655, 290656) c6( ) c7(T, am, SP, aq, K, nA, cbx, B, aDj); #c1(MAN2A1) c2(NP_002363) c3(alpha-mannosidase 2) c4(127750) c5(127751, 127752, 127753, 127754, 127755, 127756) c6( ) c7(bL, NU, bM, EQ, eZ, k, nJ); #c1(MAN2A2) c2(NP_006113) c3(alpha-mannosidase 2x) c4(162130, 162131, 162132) c5(162133, 162134, 162135, 162136, 162137, 162138, 162139, 162140, 162141, 162142, 162143, 162144) c6( ) c7(eA); #c1(MAN2A2) c2(XP_005254967) c3(alpha-mannosidase 2x isoform X1) c4(273423, 273424) c5(273425, 273426, 273427, 273428, 273429) c6( ) c7(eA); #c1(MAN2A2) c2(XP_011519866) c3(alpha-mannosidase 2x isoform X2) c4(314614) c5(314615, 314616, 314617, 314618, 314619, 314620, 314621, 314622, 314623, 314624, 314625, 314626, 314627, 314628, 314629, 314630, 314631, 314632, 314633) c6( ) c7(eA); #c1(MAN2A2) c2(XP_011519868) c3(alpha-mannosidase 2x isoform X4) c4(314634, 314635) c5(314636, 314637, 314638, 314639, 314640, 314641, 314642, 314643, 314644, 314645, 314646, 314647, 314648) c6( ) c7(eA); #c1(MAN2B1) c2(NP_000519) c3(lysosomal alpha-mannosidase isoform 1 precursor) c4(11567, 11568) c5(11569, 11570, 11571, 11572, 11573, 11574, 11575, 11576, 11577, 11578, 11579, 11580, 11581, 11582, 11583, 11584, 11585, 11586, 11587, 11588, 11589, 11590, 11591, 11592, 11593, 11594, 11595, 11596, 11597, 11598, 11599, 11600, 11601, 11602, 11603, 11604, 11605, 11606, 11607, 11608, 11609, 11610, 11611, 11612, 11613, 11614, 11615, 11616, 11617, 11618, 11619, 11620, 11621, 11622, 11623, 11624, 11625, 11626, 11627, 11628, 11629, 11630, 11631, 11632, 11633, 11634, 11635, 11636, 11637, 11638, 11639, 11640, 11641, 11642, 11643, 11644, 11645, 11646, 11647, 11648, 11649, 11650, 11651, 11652, 11653, 11654, 11655, 11656, 11657, 11658, 11659, 11660, 11661, 11662, 11663, 11664, 11665, 11666, 11667, 11668) c6(11669) c7(Lq, ey, cby); #c1(MAN2B1) c2(NP_001166969) c3(lysosomal alpha-mannosidase isoform 2 precursor) c4(68743) c5(68744, 68745, 68746, 68747, 68748, 68749) c6( ) c7(Lq, ey, cby); #c1(MAN2B1) c2(XP_005259970) c3(lysosomal alpha-mannosidase isoform X1) c4(276332) c5( ) c6( ) c7(Lq, ey, cby); #c1(MAN2B1) c2(XP_011526319) c3(lysosomal alpha-mannosidase isoform X2) c4(320611, 320612) c5(320613, 320614, 320615, 320616, 320617, 320618, 320619) c6( ) c7(Lq, ey, cby); #c1(MAN2C1) c2(NP_001243423) c3(alpha-mannosidase 201 isoform 2) c4(90736) c5(90737, 90738, 90739, 90740, 90741, 90742) c6( ) c7(By, ae, ci, ad, hu); #c1(MAN2C1) c2(NP_001243424) c3(alpha-mannosidase 2C1 isoform 3) c4(90743, 90744) c5(90745, 90746, 90747, 90748, 90749, 90750) c6( ) c7(By, ae, ci, ad, hu); #c1(MAN2C1) c2(NP_001243425) c3(alpha-mannosidase 2C1 isoform 4) c4(90751) c5(90752, 90753, 90754, 90755, 90756, 90757, 90758, 90759, 90760, 90761, 90762) c6( ) c7(By, ae, ci, ad, hu); #c1(MAN2C1) c2(NP_006706) c3(alpha-mannosidase 2C1 isoform 1) c4(167305) c5(167306, 167307, 167308, 167309, 167310, 167311) c6( ) c7(By, ae, ci, ad, hu); #c1(MAN2C1) c2(XP_011519870) c3(alpha-mannosidase 2C1 isoform X3) c4( ) c5(314649) c6( ) c7(By, ae, ci, ad, hu); #c1(MANBA) c2(NP_005899) c3(beta-mannosidase precursor) c4(160210) c5(160211, 160212, 160213, 160214, 160215, 160216) c6( ) c7(T, cbz, r, aj, Lq, cbA, V, fq, q); #c1(MANEA) c2(NP_078917) c3(glycoprotein endo-alpha-1,2-mannosidase) c4(216095, 216096, 216097) c5(216098, 216099, 216100, 216101, 216102, 216103, 216104, 216105, 216106, 216107, 216108, 216109, 216110, 216111, 216112, 216113, 216114, 216115, 216116) c6( ) c7(IB, cu, kl, VR, EQ); #c1(MANF) c2(NP_006001) c3(mesencephalic astrocyte-derived neurotrophic factor precursor) c4(161091) c5(161092, 161093, 161094, 161095, 161096, 161097, 161098, 161099) c6(161100, 161101) c7(T, aC, aE, ha, lG, j, K, fr, an, cm, cd, ad); #c1(MANSC1) c2(NP_060520) c3(MANSC domain-containing protein 1 precursor) c4(196335, 196336) c5(196337, 196338, 196339, 196340, 196341, 196342, 196343, 196344, 196345, 196346, 196347, 196348) c6( ) c7(ae, ad); #c1(MADA) c2(NP_000231) c3(amine oxidase [flavin-containing] A isoform 1) c4(5276, 5277) c5(5278, 5279, 5280, 5281, 5282, 5283, 5284) c6( ) c7(tJ, T, uK, ahc, cbF, ea, aqo, e, yY, bO, eb, gf, ur, HH, Gp, VV, x, iX, alX, K, byj, da, uY, cm, mZ, aqr, bb, abS, Gs, sv, uJ, nD, FW, dD, To, aj, ww, ct, nA, aUf, KL, FY, Mo, jz, aqJ, XY, Nn, jy, dL, mY, mV, he, mU, abf, cbC, vo, mX, hq, amt, ok, cA, yP, bhU, vL, lB, jd, B, mT, qc, bL, jv, Nt, cR, mS, Kn, v, j, wy, il, aqN, buD, Si, cF, ga, aas, HJ, bK, an, rE, ae, af, cbB, yL, iy, aPf, HL, f, FT, bN, pH, ac, cbE, aTU, OL, Fl, gx, qX, ha, bS, uu, eq, bxb, aVy, qi, ad, dP, HF, nH, w, mW, oj, cbD, na, Jc, tb, nf, gl, aTR, EZ, Z, Rm, aS, ei, pK, aqw, gq); #c1(MADA) c2(NP_001257387) c3(amine oxidase [flavin-containing] A isoform 2) c4( ) c5(95302, 95303, 95304, 95305, 95306) c6( ) c7(tJ, T, uK, ahc, cbF, ea, aqo, e, yY, bO, eb, gf, ur, HH, Gp, VV, x, iX, alX, K, byj, da, uY, cm, mZ, aqr, bb, abS, Gs, sv, uJ, nD, FW, dD, To, aj, ww, ct, nA, aUf, KL, FY, Mo, jz, aqJ, XY, Nn, jy, dL, mY, mV, he, mU, abf, cbC, vo, mX, hq, amt, ok, cA, yP, bhU, vL, lB, jd, B, mT, qc, bL, jv, Nt, cR, mS, Kn, v, j, wy, il, aqN, buD, Si, cF, ga, aas, HJ, bK, an, rE, ae, af, cbB, yL, iy, aPf, HL, f, FT, bN, pH, ac, cbE, aTU, OL, FI, gx, qX, ha, bS, uu, eq, bxb, aVy, qi, ad, dP, HF, nH, w, mW, oj, cbD, na, Jc, tb, nf, gl, aTR, EZ, Z, Rm, aS, ei, pk, aqw, gq); #c1(MAOB) c2(NP_000889) c3(amine oxidase [flavin-containing] B) c4(16851, 16852) c5(16853, 16854, 16855, 16856, 16857, 16858, 16859) c6( ) c7(bL, uK, qX, yY, bO, cR, bS, uu, ct, nA, wy, HH, qi, dP, HF, bK, K, an, he, rz, af, na, ak, abf, d, hl, aPf, HL, Z, hJ, eE, cm, bN, UP, cA, U, bhU, aS, X); #c1(MADB) c2(XP_005272664) c3(amine oxidase [flavin-containing] B isoform X1) c4(284110) c5(284111, 284112, 284113, 284114, 284115, 284116, 284117, 284118, 284119, 284120, 284121) c6( ) c7(bL, uK, qX, yY, bO, cR, bS, uu, ct, nA, wy, HH, qi, dP, HF, bK, K, an, he, rz, af, na, ak, abf, d, hl, aPf, HL, Z, hJ, eE, cm, bN, UP, cA, U, bhU, aS, X); #c1(MAP10) c2(NP_061963) c3(microtubule-associated protein 10) c4(201333) c5(201334, 201335, 201336, 201337, 201338, 201339) c6( ) c7(cd); #c1(MAP1A) c2(NP_002364) c3(microtubule-associated protein 1A) c4(127757, 127758, 127759, 127760) c5(127761, 127762, 127763, 127764, 127765, 127766, 127767, 127768, 127769, 127770, 127771, 127772, 127773, 127774, 127775, 127776, 127777, 127778, 127779, 127780, 127781, 127782, 127783, 127784, 127785, 127786, 127787) c6( ) c7(cbG); #c1(MAP1B) c2(NP_005900) c3(microtubule-associated protein (B) c4(160217) c5(160218, 160219, 160220, 160221, 160222, 160223) c6( ) c7(kf, gT, c, cm, bxw); #c1(MAP1B) c2(XP_005248564) c3(microtubule-associated protein (B isoform X1) c4( ) c5(268473) c6( ) c7(kf, gT, c, cm, bxw); #c1(MAP1LC3A) c2(NP_115903) c3(microtubule-associated proteins 1A/1B light chain 3A isoform a) c4( ) c5(224587, 224588, 224589, 224590, 224591, 224592, 224593, 224594) c6( ) c7(iu, kP, fR, aW, Dq, gX, aT, j, ci, B); #c1(MAP1LC3A) c2(NP_852610) c3(microtubule-associated proteins 1A/B light chain 3A isoform b) c4(255073) c5(255074, 255075, 255076, 255077, 255078, 255079) c6( ) c7(iu, kP, fR, aW, Dq, gX, aT, j, ci, B); #c1(MAP1LC3A) c2(XP_011527386) c3(microtubule-associated proteins 1A/1B light chain 3A isoform X1) c4(322087) c5(322088) c6( ) c7(iu, kP, fR, aW, Dq, gX, aT, j, ci, B); #c1(MAP1LC3A) c2(XP_011527387) c3(microtubule-associated proteins 1A/1B light chain 3A isoform X2) c4( ) c5(322089, 322090) c6( ) c7(iu, kP, fR, aW, Dq, gX, aT, j, ci, B); #c1(MAP1LC3B) c2(NP_073729) c3(microtubule-associated proteins (A/1B light chain 3B) c4(213291, 213292) c5(213293, 213294, 213295, 213296, 213297, 213298, 213299, 213300, 213301, 213302, 213303, 213304) c6( ) c7(T, py, kP, eM, nv, aW, eH, aT, fe, nw, j, K, nA, C, eF, rz, ra); #c1(MAP1LC3B2) c2(NP_001078950) c3(microtubule-associated proteins 1A/1B light chain 3 beta 2 precursor) c4(38155) c5(38156, 38157, 38158, 38159, 38160, 38161, 38162, 38163) c6( ) c7(hn); #c1(MAP1LC3B2) c2(XP_011536954) c3(microtubule-associated proteins 1A/1B light chain 3 beta 2 isoform X1) c4( ) c5(334641, 334642, 334643) c6( ) c7(hn); #c1(MAP1S) c2(NP_060644) c3(microtubule-associated protein (S) c4(196959) c5(196960, 196961, 196962, 196963, 196964, 196965) c6( ) c7(aW, aT, hD, h, j, an, bt); #c1(MAP2) c2(NP_001034627) c3(microtubule-associated protein 2 isoform 5) c4(31939) c5(31940, 31941, 31942, 31943, 31944, 31945) c6( ) c7(aNA, kh, My, bO, GC, il, V, eA, iC, Hx, cbH, pX, Hk, dV, aT, h, lG, bk, on, af, eu, cp, jf, asp, aW, Y, AQ, ff, en, as, cm, Hv, bN, ek, Qi, W, Dy, cA, wO, nG, hR, adR, eo, L); #c1(MAP2) c2(NP_002365) c3(microtubule-associated protein 2 isoform 1) c4(127788) c5(127789, 127790, 127791, 127792, 127793, 127794) c6( ) c7(aNA, kh, My, bO, GC, il, V, eA, iC, Hx, cbH, pX, Hk, dV, aT, h, lG, bK, on, af, eu, cp, jf, asp, aW, Y, AQ, ff, en, as, cm, Hv, bN, ek, Qi, W, Dy, cA, wO, nG, hR, adR, eo, L); #c1(MAP2) c2(NP_114033) c3(microtubule-associated protein 2 isoform 2) c4(222085) c5(222086, 222087, 222088, 222089, 222090, 222091, 222092, 222093, 222094, 222095, 222096, 222097, 222098, 222099, 222100, 222101) c6( ) c7(aNA, kh, My, bO, GC, il, V, eA, iC, Hx, cbH, pX, Hk, dV, aT, h, lG, bK, on, af, eu, cp, jf, asp, aW, Y, AQ, ff, en, as, cm, Hv, bN, ek, Qi, W, Dy, cA, wO, nG, hR, adR, eo, L); #c1(MAP2) c2(XP_011509492) c3(microtubule-associated protein 2 isoform X6) c4(300067) c5( ) c6( ) c7(aNA, kh, My, bO, GC, il, V, eA, iC, Hx, cbH, pX, Hk, dV, aT, h, lG, bK, on, af, eu, cp, jf, asp, aW, Y, AD, ff, en, as, cm, Hv, bN, ek, Qi, W, Dy, cA, wO, nG, hR, adR, eo, L); #c1(MAP2) c2(XP_011509500) c3(microtubule-associated protein 2 isoform X15) c4(300068) c5(300069, 300070, 300071, 300072, 300073, 300074) c6( ) c7(aNA, kh, My, bO, GC, il, V, eA, iC, Hx, cbH, pX, Hk, dV, aT, h, lG, bK, on, af, eu, cp, jf, asp, aW, Y, AQ, ff, en, as, cm, Hv, bN, ek, Qi, W, Dy, cA, wO, nG, hR, adR, eo, L); #c1(MAP2K1) c2(NP_002746) c3(dual specificity mitogen-activated protein kinase kinase 1) c4(131653) c5(131654, 131655, 131656, 131657, 131658, 131659) c6( ) c7(DS, T, yS, bB, ci, Qp, x, aE, ws, ja, K, eL, cp, Qe, eG, aW, wf, aEq, FS, g, as, eM, nB, iA, iT, cj, m, k, ajJ, cE, NT, qy, kv, fr, nA, jz, dL, cY, aq, W, fL, bD, B, jp, aA, yO, lu, q, eA, zn, nX, aT, cy, jj, ae, af, eu, jf, iU, Y, r, en, t, ck, aZT, bt, atS, mC, ad, dP, av, hd, ib, cbl, vc, fb, d, ff, eZ, iR, bz, es, bE, y, iu, jr); #c1(MAP2K2) c2(NP_109587) c3(dual specificity mitogen-activated protein kinase kinase 2) c4(219646) c5(219647, 219648, 219649, 219650, 219651, 219652) c6( ) c7(T, yS, atS, bB, amj, kv, j, yO, nA, q, eA, av, jz, aE, nX, cE, qK, af, Qe, jf, Y, cbJ, r, g, iV, t, bz, Qt, aZT, bE, gX, m, bD, xg, B, jr); #c1(MAP2K3) c2(NP_002747) c3(dual specificity mitogen-activated protein kinase kinase 3 isoform A) c4( ) c5(131660, 131661, 131662, 131663, 131664) c6( ) c7(ai, av, lR, eM, aW, aT, cd, v, j, K, eA, bq, y, NM, B); #c1(MAP2K3) c2(NP_659731) c3(dual specificity mitogen-activated protein kinase kinase 3 isoform B) c4(240295, 240296, 240297) c5(240298, 240299, 240300, 240301, 240302, 240303, 240304, 240305, 240306, 240307, 240308, 240309, 240310) c6( ) c7(ai, av, lR, eM, aW, aT, cd, v, j, K, eA, bq, y, NM, B); #c1(MAP2K4) c2(NP_001268364) c3(dual specificity mitogen-activated protein kinase kinase 4 isoform 2) c4(99788, 99789) c5(99790, 99791, 99792, 99793, 99794, 99795) c6( ) c7(T, aC, zX, aA, v, j, ci, q, ad, eA, bv, av, iu, aE, aT, cy, K, ae, Jl, aW, Y, eH, r, agF, vs, aF, g, sz, cl, t, eF, bz, fq, cm, ac, eM, hL, m, MC, y, bt, X); #c1(MAP2K4) c2(NP_003001) c3(dual specificity mitogen-activated protein kinase kinase 4 isoform 1) c4(134325, 134326) c5(134327, 134328, 134329, 134330, 134331, 134332, 134333, 134334, 134335, 134336, 134337, 134338) c6( ) c7(T, aC, zX, aA, v, j, ci, q, ad, eA, bv, av, iu, aE, aT, cy, K, ae, Jl, aW, Y, eH, r, agF, vs, aF, g, sz, cl, t, eF, bz, fq, cm, ac, eM, hL, m, MC, y, bt, X); #c1(MAP2K4) c2(XP_005256810) c3(dual specificity mitogen-activated protein kinase kinase 4 isoform X2) c4( ) c5(274517, 274518, 274519, 274520, 274521, 274522) c6( ) c7(T, aC, zX, aA, v, j, ci, q, ad, eA, bv, av, iu, aE, aT, cy, K, ae, Jl, aW, Y, eH, r, agF, vs, aF, g, sz, cl, t, eF, bz, fq, cm, ac, eM, hL, m, MC, y, bt, X); #c1(MAP2K5) c2(NP_001193733) c3(dual specificity mitogen-activated protein kinase kinase 5 isoform C) c4(82536) c5(82537, 82538, 82539, 82540, 82541, 82542) c6( ) c7(gh, T, eR, yY, n, bS, Jd, ct, ci, v, aY, ad, aT, ae, xa, jf, aW, yL, Z, fq, hq, mx, aS, awm); #c1(MAP2K5) c2(NP_002748) c3(dual specificity mitogen-activated protein kinase kinase 5 isoform B) c4(131665, 131666) c5(131667, 131668, 131669, 131670, 131671, 131672, 131673, 131674, 131675, 131676, 131677, 131678) c6( ) c7(gh, T, eR, yY, n, bS, Jd, ct, ci, v, aY, ad, aT, ae, xa, jf, aW, yL, Z, fq, hq, mx, aS, awm); #c1(MAP2K5) c2(NP_660143) c3(dual specificity mitogen-activated protein kinase kinase 5 isoform A) c4(240388) c5(240389, 240390, 240391, 240392, 240393, 240394) c6( ) c7(gh, T, eR, yY, n, bS, Jd, ct, ci, v, aY, ad, aT, ae, xa, jf, aW, yL, Z, fq, hq, mx, aS, awm); #c1(MAP2K6) c2(NP_002749) c3(dual specificity mitogen-activated protein kinase kinase 6) c4(131679) c5(131680, 131681, 131682, 131683, 131684, 131685, 131686, 131687, 131688, 131689) c6( ) c7(ai, rR, cc, eM, bE, fm, ds, aw, bB, cy, K, m, bq, t, bz, X, y); #c1(MAP2K7) c2(NP_001284484) c3(dual specificity mitogen-activated protein kinase kinase 7 isoform 1) c4(111888) c5(111889, 111890, 111891, 111892, 111893, 111894) c6( ) c7(bM, T, aC, yS, bO, fd, bB, lR, ci, bv, aE, pX, aeO, ja, K, eL, cp, eG, yo, aeR, aW, az, NR, g, as, cm, Qt, iP, eM, nB, Qw, cj, m, Ro, kv, zP, cd, eW, auO, jz, auj, gd, dL, oo, aq, NL, it, B, aA, q, eA, zn, nv, dn, aT, dz, jj, ae, jf, Qr, iU, Y, Pg, r, t, iK, pb, FT, ck, sS, nM, kP, Dy, np, bt, id, BD, hF, ad, abZ, dP, av, hd, ds, NS, hP, pq, d, et, aF, iV, bz, Hp, es, Ew, bE, X, y, iu); #c1(MAP2K7) c2(NP_660186) c3(dual specificity mitogen-activated protein kinase kinase 7 isoform 3) c4(240475, 240476) c5(240477, 240478, 240479, 240480, 240481, 240482, 240483, 240484, 240485, 240486, 240487, 240488) c6( ) c7(bM, T, aC, yS, bO, fd, bB, lR, ci, bv, aE, pX, aeO, ja, K, eL, cp, eG, yo, aeR, aW, az, NR, g, as, cm, lt, iP, eM, nB, Qw, cj, m, Ro, kv, zP, cd, eW, auO, jz, auj, gd, dL, oo, aq, NL, it, B, aA, q, eA, zn, nv, dn, aT, dz, jj, ae, jf, Ar, iU, Y, Pg, r, t, iK, pb, FT, ck, sS, nM, kP, Dy, np, bt, id, BD, hF, ad, abZ, dP, av, hd, ds, NS, hP, pq, d, et, aF, iV, bz, Hp, es, Ew, bE, X, y, iu); #c1(MAP3KI) c2(NP_005912) c3(mitogen-activated protein kinase kinase kinase 1) c4(160320) c5(160321, 160322, 160323, 160324, 160325, 160326) c6( ) c7(gx, T, aC, cbK, sw, aA, adW, eJ, ad, av, aE, vJ, aT, UD, K, ae, avg, A, zQ, xX, aW, Y, aF, cl, iV, t, bz, hL, m, y, B); #c1(MAP3K1) c2(XP_011541708) c3(mitogen-activated protein kinase kinase kinase 1 isoform X1) c4( ) c5(340269, 340270) c6( ) c7(gx, T, aC, cbK, sw, aA, adW, eJ, ad, av, aE, vJ, aT, UD, K, ae, avg, A, zl, xX, aW, Y, aF, cl, iV, t, bz, hL, m, y, B); #c1(MAP3K10) c2(NP_002437) c3(mitogen-activated protein kinase kinase kinase 10) c4(128461) c5(128462, 128463, 128464, 128465, 128466, 128467) c6( ) c7(x, aE, eM, bE, aW, NT, aT, bB, kp, k, pd, hq); #c1(MAP3K10) c2(XP_011525285) c3(mitogen-activated protein kinase kinase kinase 10 isoform X3) c4( ) c5(319744) c6( ) c7(x, aE, eM, bE, aW, NT, aT, bB, kp, k, pd, hq); #c1(MAP3K11) c2(NP_002410) c3(mitogen-activated protein kinase kinase kinase (1) c4(128186, 128187) c5(128188, 128189, 128190, 128191, 128192, 128193, 128194, 128195, 128196, 128197, 128198, 128199) c6( ) c7(T, jx, NT, bB, ci, q, ad, aT, h, cy, K, ae, eu, aW, r, t, ek, ac, bE, gX, U, m); #c1(MAP3K12) c2(NP_006292) c3(mitogen-activated protein kinase kinase kinase 12 isoform 2) c4(163732) c5(163733, 163734, 163735, 163736, 163737, 163738) c6( ) c7(x, iO, fM, Tb, iU, aW, r, aT, bw, k, cm, cd); #c1(MAP3K13) c2(NP_001229246) c3(mitogen-activated protein kinase kinase kinase 13 isoform 2) c4(84024) c5(84025, 84026, 84027, 84028, 84029, 84030) c6( ) c7(bL, aW, cd, r, aT); #c1(MAP3K13) c2(XP_011511612) c3(mitogen-activated protein kinase kinase kinase 13 isoform XI) c4(302345, 302346, 302347, 302348) c5(302349, 302350, 302351, 302352, 302353, 302354, 302355, 302356, 302357, 302358, 302359, 302360, 302361, 302362, 302363, 302364, 302365, 302366, 302367, 302368, 302369, 302370, 302371, 302372, 302373, 302374, 302375, 302376) c6( ) c7(bL, aW, cd, r, aT); #c1(MAP3K14) c2(XP_011523743) c3(mitogen-activated protein kinase kinase kinase 14 isoform X1) c4(318244, 318245) c5(318246, 318247, 318248, 318249, 318250, 318251, 318252, 318253, 318254, 318255, 318256, 318257) c6( ) c7(T, fN, fd, jh, aj, eW, J, ad, aE, aT, K, bc, aW, Y, aq, alJ, f, bz, bb, nM, eM, MC, y, B, hS); #c1(MAP3K19) c2(NP_001018054) c3(mitogen-activated protein kinase kinase kinase 19 isoform 3) c4(26958) c5(26959, 26960, 26961, 26962, 26963, 26964) c6( ) c7(ab, hF, fM, W); #c1(MAP3K19) c2(NP_001018056) c3(mitogen-activated protein kinase kinase kinase 19 isoform 2) c4(26965) c5(26966, 26967, 26968, 26969, 26970, 26971) c6( ) c7(ab, hF, fM, W); #c1(MAP3K19) c2(NP_001018057) c3(mitogen-activated protein kinase kinase kinase 19 isoform 4) c4(26972) c5(26973, 26974, 26975, 26976, 26977, 26978) c6( ) c7(ab, hF, fM, W); #c1(MAP3K19) c2(NP_001269812) c3(mitogen-activated protein kinase kinase kinase 19 isoform 5) c4(102894) c5(102895, 102896, 102897, 102898, 102899, 102900, 102901, 102902, 102903, 102904, 102905) c6( ) c7(ab, hF, fM, W); #c1(MAP3K19) c2(XP_011510196) c3(mitogen-activated protein kinase kinase kinase 19 isoform X3) c4(300767) c5( ) c6( ) c7(ab, hF, fM, W); #c1(MAP3K19) c2(XP_011510201) c3(mitogen-activated protein kinase kinase kinase 19 isoform X1) c4(300768, 300769) c5(300770, 300771, 300772, 300773, 300774, 300775, 300776, 300777, 300778, 300779, 300780, 300781) c6( ) c7(ab, hF, fM, W); #c1(MAP3K19) c2(XP_011510203) c3(mitogen-activated protein kinase kinase kinase 19 isoform X8) c4( ) c5(300782) c6( ) c7(ab, hF, fM, W); #c1(MAP3K2) c2(NP_006600) c3(mitogen-activated protein kinase kinase kinase 2) c4(166535, 166536) c5(166537, 166538, 166539, 166540, 166541, 166542, 166543, 166544, 166545, 166546, 166547, 166548, 166549, 166550, 166551) c6( ) c7(y, T, ci); #c1(MAP3K3) c2(NP_002392) c3(mitogen-activated protein kinase kinase kinase 3 isoform 2) c4(128083) c5(128084, 128085, 128086, 128087, 128088, 128089) c6( ) c7(T, aW, ht, aT, K, m, t); #c1(MAP3K3) c2(NP_976226) c3(mitogen-activated protein kinase kinase kinase 3 isoform 1) c4(263000) c5(263001, 263002, 263003, 263004, 263005, 263006) c6( ) c7(T, aW, ht, aT, K, m, t); #c1(MAP3K4) c2(NP_005913) c3(mitogen-activated protein kinase kinase kinase 4 isoform a) c4(160327, 160328) c5(160329, 160330, 160331, 160332, 160333, 160334) c6( ) c7(afz, hL); #c1(MAP3K4) c2(NP_006715) c3(mitogen-activated protein kinase kinase kinase 4 isoform b) c4(167364, 167365) c5(167366, 167367, 167368, 167369, 167370, 167371, 167372, 167373, 167374, 167375, 167376, 167377) c6( ) c7(afz, hL); #c1(MAP3K4) c2(XP_005267046) c3(mitogen-activated protein kinase kinase kinase 4 isoform X1) c4( ) c5(280638, 280639, 280640, 280641, 280642, 280643) c6( ) c7(afz, hL); #c1(MAP3K5) c2(NP_005914) c3(mitogen-activated protein kinase kinase kinase 5) c4(160335, 160336, 160337) c5(160338, 160339, 160340, 160341, 160342, 160343, 160344, 160345, 160346, 160347, 160348, 160349, 160350, 160351, 160352, 160353, 160354, 160355) c6( ) c7(bL, T, dD, aC, hV, ea, iW, e, aA, bB, fd, aj, dG, v, eq, mF, V, ai, J, ad, eA, cc, xE, aE, aT, cy, dL, ji, K, bq, an, ae, eL, cp, eG, aW, Y, ff, xs, eH, aMA, aq, af, en, eF, t, cm, iK, Hv, ek, ck, ac, c, iT, bE, au, U, wz, m, pw, y, bt, B); #c1(MAP3K6) c2(NP_001284538) c3(mitogen-activated protein kinase kinase kinase 6 isoform 2) c4(111995) c5(111996, 111997, 111998, 111999, 112000, 112001) c6( ) c7(rR); #c1(MAP3K6) c2(XP_011540708) c3(mitogen-activated protein kinase kinase kinase 6 isoform X1) c4(339274, 339275) c5(339276, 339277, 339278, 339279, 339280, 339281, 339282, 339283, 339284, 339285, 339286, 339287) c6( ) c7(rR); #c1(MAP3K7) c2(NP_003179) c3(mitogen-activated protein kinase kinase kinase 7 isoform A) c4(135980, 135981) c5(135982, 135983, 135984, 135985, 135986, 135987, 135988, 135989, 135990, 135991, 135992, 135993) c6( ) c7(T, bsy, lH, aw, eX, jh, fd, bB, ci, Lw, hF, ad, er, Na, cF, aE, bjA, hb, aT, eV, dL, ey, ji, ae, eL, B, aW, Y, ff, fe, aq, si, hp, en, CQ, t, Zb, fq, bz, ck, wR, cbL, m, bt, it, bsw, vQ); #c1(MAP3K7) c2(NP_663304) c3(mitogen-activated protein kinase kinase kinase 7 isoform B) c4(241164) c5(241165, 241166, 241167, 241168, 241169, 241170) c6( ) c7(T, bsy, lH, aw, eX, jh, fd, bB, ci, Lw, hF, ad, er, Na, cF, aE, bjA, hb, aT, eV, dL, ey, ji, ae, eL, B, aW, Y, ff, fe, aq, si, hp, en, CQ, t, Zb, fq, bz, ck, wR, cbL, m, bt, it, bsw, vQ); #c1(MAP3K7) c2(NP_663305) c3(mitogen-activated protein kinase kinase kinase 7 isoform C) c4(241171) c5(241172, 241173, 241174, 241175, 241176, 241177) c6( ) c7(T, bsy, lH, aw, eX, jh, fd, bB, ci, Lw, hF, ad, er, Na, cF, aE, bjA, hb, aT, eV, dL, ey, ji, ae, eL, B, aW, Y, ff, fe, aq, si, hp, en, CQ, t, Zb, fq, bz, ck, wR, cbL, m, bt, it, bsw, vQ); #c1(MAP3K7) c2(NP_663306) c3(mitogen-activated protein kinase kinase kinase 7 isoform D) c4(241178, 241179) c5(241180, 241181, 241182, 241183, 241184, 241185, 241186, 241187, 241188, 241189, 241190, 241191) c6( ) c7(T, bsy, lH, aw, eX, jh, fd, bB, ci, Lw, hF, ad, er, Na, cF, aE, bjA, hb, aT, eV, dL, ey, ji, ae, eL, B, aW, Y, ff, fe, aq, si, hp, en, CQ, t, Zb, fq, bz, ck, wR, cbL, m, bt, it, bsw, vQ); #c1(MAP3K7CL) c2(NP_001273546) c3(MAP3K7 C-terminal-like protein isoform 2) c4(105167, 105168) c5(105169, 105170, 105171, 105172, 105173, 105174, 105175, 105176, 105177, 105178, 105179, 105180, 105181, 105182, 105183, 105184, 105185, 105186, 105187, 105188, 105189, 105190, 105191, 105192, 105193, 105194, 105195, 105196, 105197, 105198, 105199) c6( ) c7(aW, aT); #c1(MAP3K7CL) c2(NP_001273551) c3(MAP3K7 C-terminal-like protein isoform 3) c4(105200) c5(105201, 105202, 105203, 105204, 105205) c6( ) c7(aW, aT); #c1(MAP3K7CL) c2(NP_001273552) c3(MAP3K7 C-terminal- like protein isoform 4) c4( ) c5(105206, 105207, 105208, 105209, 105210, 105211, 105212, 105213, 105214, 105215, 105216) c6( ) c7(aW, aT); #c1(MAP3K7CL) c2(NP_001273563) c3(MAP3K7 C-terminal-like protein isoform 1) c4(105278, 105279, 105280) c5(105281, 105282, 105283, 105284, 105285, 105286, 105287, 105288, 105289, 105290, 105291, 105292, 105293) c6( ) c7(aW, aT); #c1(MAP3 KB) c2(XP_005252421) c3(mitogen-activated protein kinase kinase kinase 8 isoform X1) c4(271741, 271742, 271743, 271744) c5(271745, 271746, 271747, 271748, 271749, 271750, 271751, 271752, 271753, 271754, 271755, 271756, 271757, 271758, 271759, 271760, 271761, 271762, 271763, 271764, 271765, 271766, 271767, 271768, 271769, 271770, 271771, 271772, 271773, 271774, 271775, 271776, 271777, 271778, 271779, 271780) c6( ) c7(DS, T, RX, fd, aA, bB, n, bZ, v, j, ci, nA, Fb, fr, Fc, pd, q, ad, eA, av, iu, hd, py, jz, pN, aT, lG, rO, bK, K, ji, ae, al, lR, TY, dH, Jl, aW, Y, r, aq, af, bu, zM, t, iK, bz, c, dB, bE, go, hL, cj, hR, m, bgi, Bh, B); #c1(MAP3K8) c2(XP_011517610) c3(mitogen-activated protein kinase kinase kinase 8 isoform X2) c4(311398) c5(311399, 311400, 311401, 311402, 311403, 311404, 311405) c6( ) c7(DS, T, RX, fd, aA, bB, n, bZ, v, j, ci, nA, Fb, fr, Fc, pd, q, ad, eA, av, iu, hd, py, jz, pN, aT, lG, rO, bk, K, ji, ae, al, lR, TY, dH, Jl, aW, Y, r, aq, aF, bu, zM, t, iK, bz, c, dB, bE, go, hL, cj, hR, m, bgi, Bh, B); #c1(MAP3K8) c2(XP_011517611) c3(mitogen-activated protein kinase kinase kinase 8 isoform X3) c4( ) c5(311406, 311407, 311408) c6( ) c7(DS, T, RX, fd, aA, bB, n, bZ, v, j, ci, nA, Fb, fr, Fc, pd, q, ad, eA, av, iu, hd, py, jz, pN, aT, lG, rO, bK, K, ji, ae, al, lR, TY, dH, Jl, aW, Y, r, aq, aF, bu, zM, t, iK, bz, c, dB, bE, go, hL, cj, hR, m, bgi, Bh, B); #c1(MAP3K9) c2(NP_001271159) c3(mitogen-activated protein kinase kinase kinase 9 isoform 2) c4(103496) c5(103497, 103498, 103499, 103500, 103501, 103502) c6( ) c7(bE, Y, ht, bB, K, hu, cp); #c1(MAP3K9) c2(NP_149132) c3(mitogen-activated protein kinase kinase kinase 9 isoform 1) c4(227392) c5(227393, 227394, 227395, 227396, 227397, 227398, 227399, 227400) c6( ) c7(bE, Y, ht, bB, K, hu, cp); #c1(MAP3K9) c2(XP_011535093) c3(mitogen-activated protein kinase kinase kinase 9 isoform X4) c4(331822) c5(331823, 331824, 331825, 331826, 331827, 331828) c6( ) c7(bE, Y, ht, bB, K, hu, cp); #c1(MAP4) c2(NP_001127836) c3(microtubule-associated protein 4 isoform 4) c4(49301) c5(49302, 49303, 49304, 49305, 49306, 49307) c6( ) c7(e, eL, oe, fM, dL); #c1(MAP4) c2(NP_002366) c3(microtubule-associated protein 4 isoform 1) c4(127795, 127796) c5(127797, 127798, 127799, 127800, 127801, 127802) c6( ) c7(e, eL, oe, fM, dL); #c1(MAP4) c2(NP_112147) c3(microtubule-associated protein 4 isoform 3) c4( ) c5(220502, 220503, 220504, 220505, 220506, 220507) c6( ) c7(e, eL, oe, fM, dL); #c1(MAP4) c2(XP_011532007) c3(microtubule-associated protein 4 isoform X3) c4(328043, 328044) c5(328045, 328046, 328047, 328048, 328049, 328050, 328051, 328052, 328053, 328054, 328055, 328056) c6( ) c7(e, eL, oe, fM, dL); #c1(MAP4) c2(XP_011532010) c3(microtubule-associated protein 4 isoform X6) c4(328057) c5(328058, 328059, 328060, 328061, 328062, 328063) c6( ) c7(e, eL, oe, fM, dL); #c1(MAP4) c2(XP_011532015) c3(microtubule-associated protein 4 isoform X12) c4(328064) c5( ) c6( ) c7(e, eL, oe, fM, dL); #c1(MAP4) c2(XP_011532020) c3(microtubule-associated protein 4 isoform X18) c4(328065) c5(328066, 328067, 328068, 328069, 328070, 328071) c6( ) c7(e, eL, oe, fM, dL); #c1(MAP4) c2(XP_011532023) c3(microtubule-associated protein 4 isoform X26) c4(328072, 328073) c5(328074, 328075, 328076, 328077, 328078, 328079, 328080, 328081, 328082, 328083, 328084, 328085, 328086, 328087, 328088, 328089, 328090, 328091) c6( ) c7(e, eL, oe, fM, dL); #c1(MAP4K1) c2(NP_001036065) c3(mitogen-activated protein kinase kinase kinase kinase 1 isoform 1) c4(33907) c5(33908, 33909, 33910, 33911, 33912, 33913, 33914, 33915, 33916, 33917, 33918, 33919) c6( ) c7(jf, aC, W, aE, bE, eZ, vs, ci, K); #c1(MAP4K1) c2(NP_009112) c3(mitogen-activated protein kinase kinase kinase kinase 1 isoform 2) c4(170772, 170773) c5(170774, 170775, 170776, 170777, 170778, 170779, 170780, 170781, 170782, 170783) c6( ) c7(jf, aC, W, aE, bE, eZ, vs, ci, K); #c1(MAP4K2) c2(NP_004570) c3(mitogen-activated protein kinase kinase kinase kinase 2) c4(147822) c5(147823, 147824, 147825, 147826, 147827, 147828) c6( ) c7(md, bL, AW, nD, mh, aC, A, bza, bAg, mg, cz, cy, aj, dG, jy, K, ei, J); #c1(MAP4K3) c2(NP_001257354) c3(mitogen-activated protein kinase kinase kinase kinase 3 isoform 2) c4(95237) c5(95238, 95239, 95240, 95241, 95242, 95243, 95244, 95245, 95246, 95247, 95248, 95249, 95250, 95251, 95252, 95253) c6( ) c7(xE, c, mi, eZ, gv, fL, mb, mC, y); #c1(MAP4K3) c2(NP_003609) c3(mitogen-activated protein kinase kinase kinase kinase 3 isoform 1) c4(139566, 139567, 139568) c5(139569, 139570, 139571, 139572, 139573, 139574, 139575, 139576) c6( ) c7(xE, c, mi, eZ, gv, fL, mb, mC, y); #c1(MAP4K4) c2(NP_001229488) c3(mitogen-activated protein kinase kinase kinase kinase 4 isoform 4) c4(84569) c5(84570, 84571, 84572, 84573, 84574, 84575, 84576, 84577, 84578, 84579, 84580, 84581) c6( ) c7(bL, T, bb, aE, eM, nM, hX, aW, Y, jh, aT, fd, aq, aj, mf, ci, bz, B, J, hS); #c1(MAP4K4) c2(NP_004825) c3(mitogen-activated protein kinase kinase kinase kinase 4 isoform 1) c4(150094) c5(150095, 150096, 150097, 150098, 150099, 150100) c6( ) c7(bL, T, bb, aE, eM, nM, hX, aW, Y, jh, aT, fd, aq, aj, mf, ci, bz, B, J, hS); #c1(MAP4K4) c2(NP_663719) c3(mitogen-activated protein kinase kinase kinase kinase 4 isoform 2) c4(241438) c5(241439, 241440, 241441, 241442, 241443, 241444) c6( ) c7(bL, T, bb, aE, eM, nM, hX, aW, Y, jh, aT, fd, aq, aj, mf, ci, bz, B, J, hS); #c1(MAP4K4) c2(XP_005264106) c3(mitogen-activated protein kinase kinase kinase kinase 4 isoform X7) c4( ) c5(278668) c6( ) c7(bL, T, bb, aE, eM, nM, hX, aW, Y, jh, aT, fd, aq, aj, mf, ci, bz, B, J, hS); #c1(MAP4K4) c2(XP_005264112) c3(mitogen-activated protein kinase kinase kinase kinase 4 isoform X14) c4(278669) c5(278670, 278671, 278672, 278673, 278674, 278675) c6( ) c7(bL, T, bb, aE, eM, nM, hX, aW, Y, jh, aT, fd, aq, aj, mf, ci, bz, B, J, hS); #c1(MAP4K4) c2(XP_005264126) c3(mitogen-activated protein kinase kinase kinase kinase 4 isoform X29) c4(278676) c5( ) c6( ) c7(bL, T, bb, aE, eM, nM, hX, aW, Y, jh, aT, fd, aq, aj, mf, ci, bz, B, J, hS); #c1(MAP4K4) c2(XP_006712932) c3(mitogen-activated protein kinase kinase kinase kinase 4 isoform X34) c4(286903) c5(286904, 286905, 286906, 286907, 286908, 286909) c6( ) c7(bL, T, bb, aE, eM, nM, hX, aW, Y, jh, aT, fd, aq, aj, mf, ci, bz, B, J, hS); #c1(MAP4K4) c2(XP_011510506) c3(mitogen-activated protein kinase kinase kinase kinase 4 isoform X19) c4(301030) c5(301031, 301032, 301033, 301034, 301035, 301036) c6( ) c7(bL, T, bb, aE, eM, nM, hX, aW, Y, jh, aT, fd, aq, aj, mf, ci, bz, B, J, hS); #c1(MAP4K4) c2(XP_011510507) c3(mitogen-activated protein kinase kinase kinase kinase 4 isoform X31) c4(301037) c5(301038, 301039, 301040, 301041, 301042, 301043) c6( ) c7(bL, T, bb, aE, eM, nM, hX, aW, Y, jh, aT, fd, aq, aj, mf, ci, bz, B, J, hS); #c1(MAP4K5) c2(XP_006720076) c3(mitogen-activated protein kinase kinase kinase kinase 5 isoform X1) c4(293204) c5(293205, 293206, 293207, 293208, 293209, 293210, 293211, 293212) c6( ) c7(bL, iM, eR, je, fc, aj, dG, j, v, ei, aY, cd, J); #c1(MAP6) c2(NP_149052) c3(microtubule-associated protein 6 isoform 1) c4(226918, 226919, 226920) c5(226921, 226922, 226923, 226924, 226925, 226926, 226927, 226928, 226929, 226930, 226931, 226932) c6( ) c7(bL, iU, aj, mf, m, fD, jg, t, Fl, q, J); #c1(MAP6) c2(NP_997460) c3(microtubule-associated protein 6 isoform 2) c4( ) c5(265536, 265537, 265538, 265539, 265540, 265541) c6( ) c7(bL, iU, aj, mf, m, fD, jg, t, Fl, q, J); #c1(MAP7) c2(NP_001185540) c3(ensconsin isoform 4) c4(75791) c5(75792, 75793, 75794, 75795, 75796, 75797) c6( ) c7(T, X, B); #c1(MAP7) c2(NP_001185543) c3(ensconsin isoform 1) c4(75798) c5(75799, 75800, 75801, 75802, 75803, 75804) c6( ) c7(T, X, B); #c1(MAP7) c2(NP_001185544) c3(ensconsin isoform 5) c4(75805) c5(75806, 75807, 75808, 75809, 75810, 75811) c6( ) c7(T, X, B); #c1(MAP7) c2(NP_001185546) c3(ensconsin isoform 7) c4(75812) c5(75813, 75814, 75815, 75816, 75817, 75818) c6( ) c7(T, X, B); #c1(MAP7) c2(NP_001185547) c3(ensconsin isoform 8) c4(75819) c5(75820, 75821, 75822, 75823, 75824) c6( ) c7(T, X, B); #c1(MAP7) c2(NP_003971) c3(ensconsin isoform 3) c4(142548) c5(142549, 142550, 142551, 142552, 142553, 142554, 142555) c6( ) c7(T, X, B); #c1(MAP9) c2(NP_001034669) c3(microtubule-associated protein 9) c4(32011, 32012, 32013, 32014) c5(32015, 32016, 32017, 32018, 32019, 32020, 32021, 32022, 32023, 32024, 32025, 32026, 32027, 32028, 32029, 32030, 32031, 32032, 32033, 32034, 32035, 32036, 32037) c6( ) c7(h); #c1(MAP9) c2(XP_006714369) c3(microtubule-associated protein 9 isoform X2) c4(287811) c5(287812, 287813, 287814, 287815, 287816, 287817, 287818) c6( ) c7(h); #c1(MAP9) c2(XP_011530557) c3(microtubule-associated protein 9 isoform X3) c4( ) c5(326482) c6( ) c7(h); #c1(MAPK1) c2(NP_620407) c3(mitogen-activated protein kinase 1) c4(237550, 237551, 237552) c5(237553, 237554, 237555, 237556, 237557, 237558, 237559, 237560, 237561, 237562, 237563, 237564, 237565, 237566, 237567, 237568, 237569, 237570, 237571, 237572, 237573, 237574) c6( ) c7(DS, gh, T, os, bM, lD, fR, aC, yS, ea, if, pf, bO, fd, bB, gf, lR, ci, Lw, x, bv, xy, xE, aE, Bc, rw, pX, aeO, M, dV, iv, K, hu, eL, cp, ly, xQ, yo, aeR, iE, aW, aEl, az, zC, g, as, eE, eF, axp, cm, Qt, bw, iP, eM, Jr, nB, iA, vA, cbM, mx, cj, NO, Qw, k, Hb, m, Xu, iK, NN, iu, Ro, NK, dD, iB, RX, ht, NT, qy, aw, NJ, aj, BF, ct, qN, fr, kv, cd, asS, ai, Hx, ft, no, jz, cc, gd, cU, h, dL, ey, ox, Bz, he, A, PX, fJ, ks, fe, aq, kp, pm, op, lx, NL, w, hL, hR, uz, eo, fL, Xa, it, B, bL, Dw, NQ, fM, dA, yk, lH, jx, aA, cR, cz, hy, v, j, C, bX, V, q, eA, eJ, zn, cF, yw, nv, qK, HC, aT, aAn, kf, cy, rD, bK, j, bq, ae, af, eu, bA, jf, hM, wr, s, NE, Qr, iU, Y, ev, afS, Pg, r, iy, cu, lo, en, hf, cl, t, iK, pb, FT, bN, ck, bvL, pa, nM, Dy, hc, cbO, gX, auN, im, bt, bFC, ao, hl, kh, MH, btY, iW, ha, jh, Hn, oZ, bS, dG, eq, hF, iJ, mC, ad, hX, dP, av, hd, F, agd, CJ, ib, yt, ds, hP, lQ, ji, cbN, fb, pq, NM, NP, d, ff, eH, cr, eZ, aoF, aF, iV, iR, bz, dr, Hp, es, D, bE, bEs, z, afT, y, WX, X, jr); #c1(MAPK10) c2(NP_002744) c3(mitogen-activated protein kinase 10 isoform 1) c4(131599, 131600, 131601) c5(131602, 131603, 131604, 131605, 131606, 131607, 131608, 131609, 131610, 131611, 131612, 131613, 131614, 131615, 131616, 131617) c6( ) c7(bM, hb, dB, cbP, h, lm, ci, K, m, nA, t, iK); #c1(MAPK10) c2(NP_620446) c3(mitogen-activated protein kinase 10 isoform 3) c4(237646, 237647, 237648, 237649) c5(237650, 237651, 237652, 237653, 237654, 237655, 237656, 237657) c6( ) c7(bM, hb, dB, cbP, h, lm, ci, K, m, nA, t, iK); #c1(MAPK10) c2(XP_005263193) c3(mitogen-activated protein kinase 10 isoform X8) c4( ) c5(278341, 278342, 278343, 278344, 278345) c6( ) c7(bM, hb, dB, cbP, h, lm, ci, K, m, nA, t, iK); #c1(MAPK10) c2(XP_006714332) c3(mitogen-activated protein kinase 10 isoform X6) c4(287690) c5(287691, 287692, 287693, 287694, 287695, 287696) c6( ) c7(bM, hb, dB, cbP, h, lm, ci, K, m, nA, t, iK); #c1(MAPK10) c2(XP_006714335) c3(mitogen-activated protein kinase 10 isoform X10) c4(287697) c5(287698, 287699, 287700, 287701, 287702, 287703, 287704, 287705, 287706, 287707, 287708, 287709, 287710, 287711, 287712, 287713, 287714, 287715, 287716, 287717, 287718, 287719, 287720, 287721, 287722, 287723, 287724, 287725, 287726, 287727, 287728, 287729, 287730, 287731, 287732, 287733, 287734, 287735, 287736, 287737, 287738, 287739, 287740, 287741, 287742, 287743, 287744, 287745, 287746, 287747, 287748, 287749, 287750, 287751, 287752, 287753, 287754, 287755, 287756, 287757) c6( ) c7(bM, hb, dB, cbP, h, lm, ci, K, m, nA, t, iK); #c1(MAPK10) c2(XP_011530421) c3(mitogen-activated protein kinase 10 isoform X5) c4(326266, 326267, 326268, 326269, 326270, 326271) c5( ) c6( ) c7(bM, hb, dB, cbP, h, lm, ci, K, m, nA, t, iK); #c1(MAPK11) c2(NP_002742) c3(mitogen-activated protein kinase 11) c4(131579, 131580) c5(131581, 131582, 131583, 131584, 131585, 131586, 131587, 131588, 131589, 131590, 131591) c6( ) c7(ck, cc, d, eM, h, K, y, B); #c1(MAPK11) c2(XP_011529008) c3(mitogen-activated protein kinase Il isoform X1) c4( ) c5(324369, 324370, 324371, 324372, 324373) c6( ) c7(ck, cc, d, eM, h, K, y, B); #c1(MAPK12) c2(NP_001290181) c3(mitogen-activated protein kinase (2 isoform 2) c4(115100) c5(115101, 115102, 115103, 115104, 115105, 115106) c6( ) c7(T, bE, aW, Y, ff, aT, bB, ci, en, bz); #c1(MAPK12) c2(NP_002960) c3(mitogen-activated protein kinase 12 isoform 1) c4(133938) c5(133939, 133940, 133941, 133942, 133943, 133944) c6( ) c7(T, bE, aW, Y, ff, aT, bB, ci, en, bz); #c1(MAPK13) c2(NP_002745) c3(mitogen-activated protein kinase 13) c4(131618, 131619, 131620, 131621, 131622) c5(131623, 131624, 131625, 131626, 131627, 131628, 131629, 131630, 131631, 131632, 131633, 131634, 131635, 131636, 131637, 131638, 131639, 131640, 131641, 131642, 131643, 131644, 131645, 131646, 131647, 131648, 131649, 131650, 131651, 131652) c6( ) c7(jz, nO, h, K, bjl, hJ, y, rY); #c1(MAPK14) c2(NP_001306) c3(mitogen-activated protein kinase 14 isoform 1) c4(116556) c5(116557, 116558, 116559, 116560, 116561, 116562) c6( ) c7(bM, T, aC, fR, ea, if, fd, bB, gf, lR, ci, Lw, bv, xE, aE, rw, dV, K, hu, H, pk, eL, YL, aW, az, zC, g, as, hJ, eF, cm, bw, eM, nB, iT, cj, NO, Hb, m, NN, iu, NK, dD, ht, RC, NJ, aj, BF, ct, qN, asF, fr, cd, ai, Hx, jz, cc, cU, lG, jy, dL, h, fe, aq, NL, hk, w, hR, at, B, L, bL, Dw, NQ, fM, yk, lH, jx, aA, cR, cz, v, j, C, V, q, J, eA, eJ, cF, yw, uT, aT, kf, cy, ae, af, hM, wr, iU, Y, DN, Pg, r, lo, en, t, iK, pb, ck, c, mf, im, bt, ao, kh, jh, bS, dG, eq, iJ, ad, hX, dP, av, CJ, F, ib, ds, Aa, lQ, jo, ji, fb, NM, NP, d, ff, eH, cr, aF, Z, jb, bz, xZ, bE, aDB, ME, y, X); #c1(MAPK14) c2(NP_620581) c3(mitogen-activated protein kinase 14 isoform 2) c4(237783) c5(237784, 237785, 237786, 237787, 237788, 237789, 237790) c6( ) c7(bM, T, aC, fR, ea, if, fd, bB, gf, lR, ci, Lw, bv, xE, aE, rw, dV, K, hu, H, pk, eL, YL, aW, az, zC, g, as, hJ, eF, cm, bw, eM, nB, iT, cj, NO, Hb, m, NN, iu, NK, dD, ht, RC, NJ, aj, BF, ct, qN, asF, fr, cd, ai, Hx, jz, cc, cU, IG, jy, dL, h, fe, aq, NL, hk, w, hR, at, B, L, bL, Dw, NQ, fM, yk, lH, jx, aA, CR, cz, v, j, C, V, q, J, eA, eJ, cF, yw, uT, aT, kf, cy, ae, af, hM, wr, iU, Y, DN, Pg, r, lo, en, t, iK, pb, ck, c, mf, im, bt, ao, kh, jh, bS, dG, eq, iJ, ad, hX, dP, av, CJ, F, ib, ds, Aa, lQ, jo, ji, fb, NM, NP, d, ff, eH, cr, aF, Z, jb, bz, xZ, bE, aDB, ME, y, X); #c1(MAPK14) c2(NP_620583) c3(mitogen-activated protein kinase 14 isoform 4) c4(237791) c5(237792, 237793, 237794, 237795, 237796, 237797) c6( ) c7(bM, T, aC, fR, ea, if, fd, bB, gf, lR, ci, Lw, bv, xE, aE, rw, dV, K, hu, H, pk, eL, YL, aW, az, zC, g, as, hJ, eF, cm, bw, eM, nB, iT, cj, NO, Hb, m, NN, iu, NK, dD, ht, RC, NJ, aj, BF, ct, qN, asf, fr, cd, ai, Hx, jz, cc, cU, lG, jy, dL, h, fe, aq, NL, hk, w, hR, at, B, L, bL, Dw, NQ, fM, yk, lH, jx, aA, cR, cz, v, j, C, V, q, J, eA, eJ, cF, yw, uT, aT, kf, cy, ae, af, hM, wr, iU, Y, DN, Pg, r, lo, en, t, ik, pb, ck, c, mf, im, bt, ao, kh, jh, bS, dG, eq, iJ, ad, hX, dP, av, CJ, F, ib, ds, Aa, lQ, jo, ji, fb, NM, NP, d, ff, eH, cr, aF, Z, jb, bz, xZ, bE, aDB, ME, y, X); #c1(MAPK14) c2(XP_006715061) c3(mitogen-activated protein kinase 14 isoform X3) c4( ) c5(288562, 288563, 288564, 288565, 288566) c6( ) c7(bM, T, aC, fR, ea, if, fd, bB, gf, lR, ci, Lw, bv, xE, aE, rw, dV, K, hu, H, pk, eL, YL, aW, az, zC, g, as, hJ, eF, cm, bw, eM, nB, iT, cj, NO, Hb, m, NN, iu, NK, dD, ht, RC, NJ, aj, BF, ct, qN, asf, fr, cd, ai, Hx, jz, cc, cU, lG, jy, dL, h, fe, aq, NL, hk, w, hR, at, B, L, bL, Dw, NQ, fM, yk, lH, jx, aA, cR, cz, v, j, C, V, q, J, eA, eJ, cF, yw, uT, aT, kf, cy, ae, af, hM, wr, iU, Y, DN, Pg, r, lo, en, t, iK, pb, ck, c, mf, im, bt, ao, kh, jh, bS, dG, eq, iJ, ad, hX, dP, av, CJ, F, ib, ds, Aa, lQ, jo, ji, fb, NM, NP, d, ff, eH, cr, aF, Z, jb, bz, xZ, bE, aDB, ME, y, X); #c1(MAPK14) c2(XP_011512612) c3(mitogen-activated protein kinase 14 isoform X1) c4(303577) c5( ) c6( ) c7(bM, T, aC, fR, ea, if, fd, bB, gf, lR, ci, Lw, bv, xE, aE, rw, dV, K, hu, H, pk, eL, YL, aW, az, zC, g, as, hJ, eF, cm, bw, eM, nB, iT, cj, NO, Hb, m, NN, iu, NK, dD, ht, RC, NJ, aj, BF, ct, qN, asf, fr, cd, ai, Hx, jz, cc, cU, lG, jy, dL, h, fe, aq, NL, hk, w, hR, at, B, L, bL, Dw, NQ, fM, yk, lH, jx, aA, cR, cz, v, j, C, V, q, J, eA, eJ, cF, yw, uT, aT, kf, cy, ae, af, hM, wr, iU, Y, DN, Pg, r, lo, en, t, iK, pb, ck, c, mf, im, bt, ao, kh, jh, bS, dG, eq, iJ, ad, hX, dP, av, CJ, F, ib, ds, Aa, lQ, jo, ji, fb, NM, NP, d, ff, eH, cr, aF, Z, jb, bz, xZ, bE, aDB, ME, y, X); #c1(MAPK15) c2(NP_620590) c3(mitogen-activated protein kinase 15) c4(237825, 237826, 237827) c5(237828, 237829, 237830, 237831, 237832, 237833, 237834, 237835, 237836, 237837, 237838, 237839, 237840, 237841, 237842, 237843, 237844, 237845, 237846, 237847, 237848, 237849, 237850, 237851) c6(237852, 237853, 237854, 237855, 237856, 237857) c7(aW, aT); #c1(MAPK3) c2(NP_001035145) c3(mitogen-activated protein kinase 3 isoform 2) c4(32510) c5(32511, 32512, 32513, 32514, 32515, 32516) c6( ) c7(ID, gh, T, bM, aC, fR, yS, ea, fd, bB, ci, Lw, ip, aE, ws, K, hH, ass, eL, Qe, aa, ly, aW, aEl, Ls, fc, g, as, pL, eF, axp, cm, bw, iP, qJ, eM, mx, vy, cj, m, dD, iB, aw, ht, NT, fg, n, ct, asS, ft, jz, cc, h, eV, dL, ks, aq, kp, dw, jT, lx, bm, amb, bD, B, fM, dA, eX, aA, hy, j, yO, C, q, eA, XX, BJ, qK, HC, aCV, aT, bZk, rD, bK, jj, ae, af, eu, bA, jf, hM, bf, s, iU, Y, r, cu, en, cl, t, iK, pb, bN, ck, nM, Dy, mf, bt, hS, BD, ob, MH, df, jh, wV, bS, eq, oH, iJ, ad, dP, av, ib, py, yt, sN, bnM, Os, hP, NM, Yi, d, ff, eH, cr, dN, aF, Z, bz, bF, es, D, yE, bE, cP, ei, lC, X, y); #c1(MAPK3) c2(NP_001103361) c3(mitogen-activated protein kinase 3 isoform 3) c4(41896) c5(41897, 41898, 41899, 41900, 41901, 41902, 41903, 41904, 41905, 41906) c6( ) c7(ID, gh, T, bM, aC, fR, yS, ea, fd, bB, ci, Lw, ip, aE, ws, K, hH, ass, eL, Qe, aa, ly, aW, aEl, Ls, fc, g, as, pL, eF, axp, cm, bw, iP, qJ, eM, mx, vy, cj, m, dD, iB, aw, ht, NT, fg, n, ct, asS, ft, jz, cc, h, eV, dL, ks, aq, kp, dw, jT, lx, bm, amb, bD, B, fM, dA, eX, aA, hy, j, yO, C, q, eA, XX, BJ, qK, HC, aCV, aT, bZk, rD, bK, jj, ae, af, eu, bA, jf, hM, bf, s, iU, Y, r, cu, en, cl, t, iK, pb, bN, ck, nM, Dy, mf, bt, hS, BD, ob, MH, df, jh, wV, bS, eq, oH, iJ, ad, dP, av, ib, py, yt, sN, bnM, Os, hP, NM, Yi, d, ff, eH, cr, dN, aF, Z, bz, bF, es, D, yE, bE, cP, ei, lC, X, y); #c1(MAPK3) c2(NP_002737) c3(mitogen-activated protein kinase 3 isoform 1) c4(131535, 131536, 131537) c5(131538, 131539, 131540, 131541, 131542, 131543, 131544, 131545, 131546, 131547, 131548, 131549, 131550, 131551, 131552, 131553, 131554, 131555) c6( ) c7(ID, gh, T, bM, aC, fR, yS, ea, fd, bB, ci, Lw, ip, aE, ws, K, hH, ass, eL, de, aa, ly, aW, aEl, Ls, fc, g, as, pL, eF, axp, cm, bw, iP, qJ, eM, mx, vy, cj, m, dD, iB, aw, ht, NT, fg, n, ct, asS, ft, jz, cc, h, eV, dL, ks, aq, kp, dw, jT, lx, bm, amb, bD, B, fM, dA, eX, aA, hy, j, yO, C, q, eA, XX, BJ, qK, HC, aCV, aT, bZk, rD, bK, jj, ae, af, eu, bA, jf, hM, bf, s, iU, Y, r, cu, en, cl, t, iK, pb, bN, ck, nM, Dy, mf, bt, hS, BD, ob, MH, df, jh, wV, bS, eq, oH, iJ, ad, dP, av, ib, py, yt, sN, bnM, Os, hP, NM, Yi, d, ff, eH, cr, dN, af, Z, bz, bF, es, D, yE, bE, cP, ei, lC, X, y); #c1(MAPK6) c2(XP_005254596) c3(mitogen-activated protein kinase 6 isoform X1) c4(273272) c5(273273, 273274, 273275, 273276, 273277, 273278) c6( ) c7(T, Y, ff, ci, en, bz); #c1(MAPK7) c2(NP_002740) c3(mitogen-activated protein kinase 7 isoform 1) c4(131556, 131557, 131558) c5(131559, 131560, 131561, 131562, 131563, 131564, 131565, 131566, 131567, 131568, 131569, 131570, 131571, 131572, 131573, 131574, 131575, 131576, 131577, 131578) c6( ) c7(T, dD, aZ, fG, e, ea, eX, fd, cR, bB, aj, bS, eq, ci, J, ad, av, ib, py, aT, eV, dL, K, ae, eL, Qe, jf, aW, ff, eH, en, eF, bz, bN, ck, iT, bE, gX, aS, B, hS); #c1(MAPK7) c2(XP_006721622) c3(mitogen-activated protein kinase 7 isoform X2) c4( ) c5(294119) c6( ) c7(T, dD, aZ, fG, e, ea, eX, fd, cR, bB, aj, bS, eq, ci, J, ad, av, ib, py, aT, eV, dL, K, ae, eL, Qe, jf, aW, ff, eH, en, eF, bz, bN, ck, iT, bE, gX, aS, B, hS); #c1(MAPK7) c2(XP_011522259) c3(mitogen-activated protein kinase 7 isoform X1) c4( ) c5(316909, 316910, 316911, 316912, 316913) c6( ) c7(T, dD, aZ, fG, e, ea, eX, fd, cR, bB, aj, bS, eq, ci, J, ad, av, ib, py, aT, eV, dL, K, ae, eL, Qe, jf, aW, ff, eH, en, eF, bz, bN, ck, iT, bE, gX, aS, B, hS); #c1(MAPK8) c2(NP_001265477) c3(mitogen-activated protein kinase 8 isoform 5) c4(98635, 98636, 98637, 98638) c5(98639, 98640, 98641, 98642, 98643, 98644, 98645, 98646, 98647, 98648, 98649, 98650, 98651, 98652, 98653, 98654, 98655, 98656, 98657, 98658, 98659, 98660, 98661, 98662, 98663, 98664, 98665, 98666) c6( ) c7(bM, T, os, fd, bB, ci, fw, bv, aE, Bc, rw, JT, K, aa, cbl, eG, aW, fc, hl, CT, La, as, eE, eF, cm, bw, eM, iA, iT, U, cj, Hb, m, Xu, aCS, NT, aj, cd, br, ai, aGR, Hx, ft, zw, h, lG, jy, bl, dx, ia, A, fe, aq, mo, Zb, bP, dB, NL, bo, bm, hR, GN, eo, Pr, B, arO, bL, fM, Dh, lH, aA, cz, v, j, C, AO, V, q, J, eA, bke, aT, cy, an, ae, af, eu, jf, bf, iU, Y, r, en, t, bN, ck, ac, c, gX, EY, fD, bt, kh, qb, iW, jh, oZ, dG, jE, iJ, ad, hX, av, ib, ds, bj, ji, d, qt, ff, xs, eH, dq, aF, iR, KM, bz, es, bE, aRB, wz, ME, cP, ei, hE, X, y); #c1(MAPK8) c2(XP_011538257) c3(mitogen-activated protein kinase 8 isoform X4) c4(335863) c5(335864, 335865, 335866, 335867, 335868, 335869) c6( ) c7(bM, T, os, fd, bB, ci, fw, bv, aE, Bc, rw, JT, K, aa, cbl, eG, aW, fc, hl, CT, La, as, eE, eF, cm, bw, eM, iA, iT, U, cj, Hb, m, Xu, aCS, NT, aj, cd, br, ai, aGR, Hx, ft, zw, h, lG, jy, bl, dx, ia, A, fe, aq, mo, Zb, bP, dB, NL, bo, bm, HR, GN, eo, Pr, B, arO, bL, fM, Dh, lH, aA, cz, v, j, C, AO, V, q, J, eA, bke, aT, cy, an, ae, af, eu, jf, bf, iU, Y, r, en, t, bN, ck, ac, c, gX, EY, fD, bt, kh, qb, iW, jh, oZ, dG, jE, iJ, ad, hX, av, ib, ds, bj, ji, d, qt, ff, xs, eH, dq, aF, iR, KM, bz, es, bE, aRB, wz, ME, cP, ei, hE, X, y); #c1(MAPK8) c2(XP_011538258) c3(mitogen-activated protein kinase 8 isoform X3) c4(335870) c5(335871, 335872, 335873, 335874, 335875, 335876, 335877, 335878, 335879, 335880) c6( ) c7(bM, T, os, fd, bB, ci, fw, bv, aE, Bc, rw, JT, K, aa, cbQ, eG, aW, fc, hl, CT, La, as, eE, eF, cm, bw, eM, iA, iT, U, cj, Hb, m, Xu, aCS, NT, aj, cd, br, ai, aGR, Hx, ft, zw, h, lG, jy, bl, dx, ia, A, fe, aq, mo, Zb, bP, dB, NL, bo, bm, hR, GN, eo, Pr, B, arO, bL, fM, Dh, lH, aA, cz, v, j, C, AO, V, q, J, eA, bke, aT, cy, an, ae, af, eu, jf, bf, iU, Y, r, en, t, bN, ck, ac, c, gX, EY, fD, bt, kh, qb, iW, jh, oZ, dG, jE, iJ, ad, hX, av, ib, ds, bj, ji, d, qt, ff, xs, eH, dq, aF, iR, KM, bz, es, bE, aRB, wz, ME, cP, ei, hE, X, y); #c1(MAPK8) c2(XP_011538259) c3(mitogen-activated protein kinase 8 isoform X2) c4(335881) c5(335882, 335883, 335884, 335885, 335886, 335887) c6( ) c7(bM, T, os, fd, bB, ci, fw, bv, aE, Bc, rw, JT, K, aa, cbQ, eG, aW, fc, hl, CT, La, as, eE, eF, cm, bw, eM, iA, iT, U, cj, Hb, m, Xu, aCS, NT, aj, cd, br, ai, aGR, Hx, ft, zw, h, lG, jy, bl, dx, ia, A, fe, aq, mo, Zb, bP, dB, NL, bo, bm, hR, GN, eo, Pr, B, arO, bL, fM, Dh, lH, aA, cz, v, j, C, AD, V, q, J, eA, bke, aT, cy, an, ae, af, eu, jf, bf, iU, Y, r, en, t, bN, ck, ac, c, gX, EY, fD, bt, kh, qb, iW, jh, oZ, dG, jE, iJ, ad, hX, av, ib, ds, bj, ji, d, qt, ff, xs, eH, dq, aF, iR, KM, bz, es, bE, aRB, wz, ME, cP, ei, hE, X, y); #c1(MAPK8) c2(XP_011538260) c3(mitogen-activated protein kinase 8 isoform X1) c4(335888) c5(335889, 335890, 335891, 335892, 335893, 335894) c6( ) c7(bM, T, os, fd, bB, ci, fw, bv, aE, Bc, rw, JT, K, aa, cbQ, eG, aW, fc, hl, CT, La, as, eE, eF, cm, bw, eM, iA, iT, U, cj, Hb, m, Xu, aCS, NT, aj, cd, br, ai, aGR, Hx, ft, zw, h, lG, jy, bl, dx, ia, A, fe, aq, mo, Zb, bP, dB, NL, bo, bm, hR, GN, eo, Pr, B, arO, bL, fM, Dh, lH, aA, cz, v, j, C, AO, V, q, J, eA, bke, aT, cy, an, ae, af, eu, jf, bf, iU, Y, r, en, t, bN, ck, ac, c, gX, EY, fD, bt, kh, qb, iW, jh, oZ, dG, jE, iJ, ad, hX, av, ib, ds, bj, ji, d, qt, ff, xs, eH, dq, af, iR, KM, bz, es, bE, aRB, wz, ME, cP, ei, hE, X, y); #c1(MAPK8IP1) c2(NP_005447) c3(C-Jun-amino-terminal kinase-interacting protein 1) c4(156276, 156277) c5(156278, 156279, 156280, 156281, 156282, 156283) c6( ) c7(bL, mj, eH, aj, bu, eF, er, af, J, ad); #c1(MAPK8IP1) c2(XP_005253283) c3(C-Jun-amino-terminal kinase-interacting protein 1 isoform X1) c4( ) c5(272368, 272369, 272370, 272371, 272372, 272373, 272374, 272375, 272376, 272377, 272378, 272379) c6( ) c7(bL, mj, eH, aj, bu, eF, er, af, J, ad); #c1(MAPK8IP2) c2(NP_036456) c3(C-Jun-amino-terminal kinase-interacting protein 2) c4(174018, 174019) c5(174020, 174021, 174022, 174023, 174024, 174025) c6( ) c7(qN, if, xg, fb, bN); #c1(MAPK8IP2) c2(XP_011528982) c3(C-Jun-amino-terminal kinase-interacting protein 2 isoform X2) c4(324323, 324324, 324325) c5( ) c6( ) c7(qN, if, xg, fb, bN); #c1(MAPK8IP2) c2(XP_011528983) c3(C-Jun-amino-terminal kinase-interacting protein 2 isoform X3) c4( ) c5(324326, 324327, 324328, 324329, 324330, 324331, 324332, 324333, 324334, 324335, 324336, 324337, 324338, 324339, 324340, 324341, 324342, 324343, 324344, 324345, 324346, 324347, 324348, 324349, 324350, 324351, 324352, 324353, 324354, 324355) c6( ) c7(qN, if, xg, fb, bN); #c1(MAPK8IP3) c2(NP_001035529) c3(C-Jun-amino-terminal kinase-interacting protein 3 isoform 2) c4(32860) c5(32861, 32862, 32863, 32864, 32865, 32866) c6( ) c7(dB, lG, T, kl, bK); #c1(MAPK8IP3) c2(NP_055948) c3(C-Jun-amino-terminal kinase-interacting protein 3 isoform 1) c4(184609) c5(184610, 184611, 184612, 184613, 184614, 184615) c6( ) c7(dB, lG, T, kl, bK); #c1(MAPK9) c2(NP_001128516) c3(mitogen-activated protein kinase 9 isoform JNK2 gamma) c4(50145) c5(50146, 50147, 50148, 50149, 50150, 50151, 50152) c6( ) c7(T, fd, bB, v, ci, cd, aR, ad, eA, ft, Bc, JT, aT, lG, cy, jy, h, K, ae, d, B, iU, aW, ff, en, as, cm, bz, ck, c, iT, bE, hk, U, m, y, X); #c1(MAPK9) c2(NP_002743) c3(mitogen-activated protein kinase 9 isoform alpha2) c4(131592) c5(131593, 131594, 131595, 131596, 131597, 131598) c6( ) c7(T, fd, bB, v, ci, cd, aR, ad, eA, ft, Bc, JT, aT, lG, cy, jy, h, K, ae, d, B, iU, aW, ff, en, as, cm, bz, ck, c, iT, bE, hk, U, m, y, X); #c1(MAPK9) c2(NP_620707) c3(mitogen-activated protein kinase 9 isoform alpha1) c4(238066) c5(238067, 238068, 238069, 238070, 238071, 238072, 238073) c6( ) c7(T, fd, bB, v, ci, cd, aR, ad, eA, ft, Bc, JT, aT, lG, cy, jy, h, K, ae, d, B, iU, aW, ff, en, as, cm, bz, ck, c, iT, bE, hk, U, m, y, X); #c1(MAPK9) c2(NP_620708) c3(mitogen-activated protein kinase 9 isoform beta1) c4(238074, 238075) c5(238076, 238077, 238078, 238079, 238080, 238081) c6( ) c7(T, fd, bB, v, ci, cd, aR, ad, eA, ft, Bc, JT, aT, lG, cy, jy, h, K, ae, d, B, iU, aW, ff, en, as, cm, bz, ck, c, iT, bE, hk, U, m, y, X); #c1(MAPK9) c2(NP_620709) c3(mitogen-activated protein kinase 9 isoform beta2) c4(238082, 238083) c5(238084, 238085, 238086, 238087, 238088, 238089) c6( ) c7(T, fd, bB, v, ci, cd, aR, ad, eA, ft, Bc, JT, aT, lG, cy, jy, h, K, ae, d, B, iU, aW, ff, en, as, cm, bz, ck, c, iT, bE, hk, U, m, y, X); #c1(MAPKAP1) c2(NP_001006618) c3(target of rapamycin complex 2 subunit MAPKAP1 isoform 1) c4(22217, 22218) c5(22219, 22220, 22221, 22222, 22223, 22224, 22225, 22226, 22227, 22228, 22229, 22230) c6( ) c7(ck, Na, T, zw, ac, awN, Qu, fd, h, lo, Nc, fr, K, eA, kC, B, alk); #c1(MAPKAP1) c2(NP_001006619) c3(target of rapamycin complex 2 subunit MAPKAP1 isoform 5) c4(22231) c5(22232, 22233, 22234, 22235, 22236, 22237, 22238, 22239, 22240, 22241, 22242, 22243, 22244, 22245, 22246, 22247, 22248, 22249) c6( ) c7(ck, Na, T, zw, ac, awN, Qu, fd, h, lo, Nc, fr, K, eA, kC, B, alk); #c1(MAPKAP1) c2(NP_001006620) c3(target of rapamycin complex 2 subunit MAPKAP1 isoform 3) c4(22250) c5(22251, 22252, 22253, 22254, 22255, 22256) c6( ) c7(ck, Na, T, zw, ac, awN, Qu, fd, h, lo, Nc, fr, K, eA, kC, B, alk); #c1(MAPKAP1) c2(NP_077022) c3(target of rapamycin complex 2 subunit MAPKAP1 isoform 2) c4(214775, 214776) c5(214777, 214778, 214779, 214780, 214781, 214782, 214783, 214784, 214785, 214786, 214787, 214788) c6( ) c7(ck, Na, T, zw, ac, awN, Qu, fd, h, lo, Nc, fr, K, eA, kC, B, alk); #c1(MAPKAP1) c2(XP_011517307) c3(target of rapamycin complex 2 subunit MAPKAP1 isoform X2) c4(310973) c5(310974) c6( ) c7(ck, Na, T, zw, ac, awN, Qu, fd, h, lo, Nc, fr, K, eA, kC, B, alk); #c1(MAPKAP1) c2(XP_011517308) c3(target of rapamycin complex 2 subunit MAPKAP1 isoform X3) c4(310975, 310976) c5( ) c6( ) c7(ck, Na, T, zw, ac, awN, Qu, fd, h, lo, Nc, fr, K, eA, kC, B, alk); #c1(MAPKAP1) c2(XP_011517311) c3(target of rapamycin complex 2 subunit MAPKAP1 isoform X6) c4( ) c5(310977, 310978, 310979, 310980, 310981) c6( ) c7(ck, Na, T, zw, ac, awN, Qu, fd, h, lo, Nc, fr, K, eA, kC, B, alk); #c1(MAPKAP1) c2(XP_011517312) c3(target of rapamycin complex 2 subunit MAPKAP1 isoform X1) c4(310982) c5(310983, 310984, 310985, 310986, 310987, 310988, 310989, 310990, 310991, 310992, 310993, 310994, 310995, 310996) c6( ) c7(ck, Na, T, zw, ac, awN, Qu, fd, h, lo, Nc, fr, K, eA, kC, B, alk); #c1(MAPKAPK2) c2(NP_004750) c3(MAP kinase-activated protein kinase 2 isoform 1) c4(149255) c5(149256, 149257, 149258, 149259, 149260, 149261, 149262) c6( ) c7(ae, T, fb, aE, eM, eL, cr, aT, fd, tv, dL, tu, K, kp, y, bz, af, ad); #c1(MAPKAPK2) c2(NP_116584) c3(MAP kinase-activated protein kinase 2 isoform 2) c4(226294) c5(226295, 226296, 226297, 226298, 226299, 226300) c6( ) c7(ae, T, fb, aE, eM, eL, cr, aT, fd, tv, dL, tu, K, kp, y, bz, af, ad); #c1(MAPKAPK3) c2(NP_001230855) c3(MAP kinase-activated protein kinase 3) c4(86841, 86842, 86843, 86844) c5(86845, 86846, 86847, 86848, 86849, 86850, 86851, 86852, 86853, 86854, 86855, 86856, 86857, 86858, 86859, 86860, 86861, 86862, 86863, 86864, 86865, 86866) c6( ) c7(F, K, vi); #c1(MAPKBP1) c2(NP_001122080) c3(mitogen-activated protein kinase-binding protein 1 isoform b) c4(46395) c5(46396, 46397, 46398, 46399, 46400, 46401) c6( ) c7(bX); #c1(MAPKBP1) c2(NP_001252540) c3(mitogen-activated protein kinase-binding protein 1 isoform c) c4(94219) c5(94220, 94221, 94222, 94223, 94224, 94225) c6( ) c7(bX); #c1(MAPKBP1) c2(NP_055809) c3(mitogen-activated protein kinase-binding protein 1 isoform a) c4(183642) c5(183643, 183644, 183645, 183646, 183647, 183648, 183649, 183650, 183651, 183652, 183653, 183654) c6( ) c7(bX); #c1(MAPKBP1) c2(XP_006720501) c3(mitogen-activated protein kinase-binding protein 1 isoform X3) c4(293534, 293535) c5(293536, 293537, 293538, 293539, 293540, 293541) c6( ) c7(bX); #c1(MAPKBP1) c2(XP_011519685) c3(mitogen-activated protein kinase-binding protein 1 isoform X2) c4(314360) c5( ) c6( ) c7(bX); #c1(MAPKBP1) c2(XP_011519686) c3(mitogen-activated protein kinase-binding protein 1 isoform X4) c4( ) c5(314361, 314362) c6( ) c7(bX); #c1(MAPKBP1) c2(XP_011519687) c3(mitogen-activated protein kinase-binding protein 1 isoform X5) c4( ) c5(314363, 314364, 314365, 314366, 314367, 314368) c6( ) c7(bX); #c1(MAPRE1) c2(NP_036457) c3(microtubule-associated protein RP/EB family member 1) c4(174026) c5(174027, 174028, 174029, 174030, 174031, 174032) c6( ) c7(bF, ou, T, eM, aW, r, aT, n, j, bz, B); #c1(MAPRE2) c2(NP_001137298) c3(microtubule-associated protein RP/EB family member 2 isoform 2) c4(55367) c5(55368, 55369, 55370, 55371, 55372) c6( ) c7(ck, bF, ou, T, xX, lR, aE, eM, ff, an, r, n, nw, q, en, nk, Kw, nC, B, bA); #c1(MAPRE2) c2(NP_001137299) c3(microtubule-associated protein RP/EB family member 2 isoform 3) c4(55373) c5(55374, 55375, 55376, 55377, 55378, 55379) c6( ) c7(ck, bF, ou, T, xX, lR, aE, eM, ff, an, r, n, nw, q, en, nk, Kw, nC, B, bA); #c1(MAPRE2) c2(NP_001243349) c3(microtubule-associated protein RP/EB family member 2 isoform 4) c4(90464) c5(90465, 90466, 90467, 90468, 90469, 90470) c6( ) c7(ck, bF, ou, T, xX, lR, aE, eM, ff, an, r, n, nw, q, en, nk, Kw, nC, B, bA); #c1(MAPRE2) c2(NP_055083) c3(microtubule-associated protein RP/EB family member 2 isoform 1) c4(178860) c5(178861, 178862, 178863, 178864, 178865, 178866) c6( ) c7(ck, bF, ou, T, xX, lR, aE, eM, ff, an, r, n, nw, q, en, nk, Kw, nC, B, bA); #c1(MAPRE2) c2(XP_006722438) c3(microtubule-associated protein RP/EB family member 2 isoform X2) c4( ) c5(294711) c6( ) c7(ck, bF, ou, T, xX, lR, aE, eM, ff, an, r, n, nw, q, en, nk, Kw, nC, B, bA); #c1(MAPRE3) c2(NP_001289979) c3(microtubule-associated protein RP/EB family member 3) c4(114905, 114906) c5(114907, 114908, 114909, 114910, 114911, 114912, 114913, 114914, 114915, 114916, 114917, 114918) c6( ) c7(jf, afR, T, kl, dB, yg, aA, Be, aF, HU, ayj, nC); #c1(MAPRE3) c2(XP_011531002) c3(microtubule-associated protein RP/EB family member 3 isoform X1) c4(326954) c5(326955, 326956, 326957, 326958, 326959, 326960, 326961, 326962, 326963, 326964) c6( ) c7(jf, afR, T, kl, dB, yg, aA, Be, aF, HU, ayj, nC); #c1(MAPT) c2(NP_001116538) c3(microtubule-associated protein tau isoform 6) c4(43908, 43909) c5(43910, 43911, 43912, 43913, 43914, 43915, 43916, 43917, 43918, 43919, 43920, 43921, 43922, 43923, 43924, 43925) c6( ) c7(T, FR, bUk, e, Vd, aPl, aR, x, Gt, Fo, aU, K, H, li, aW, dg, cm, bb, jV, cbT, U, k, R, m, act, bmE, cZ, dt, dD, Ol, aw, nA, PJ, Mo, Hx, no, bl, cbU, dd, acM, aWO, dB, cbS, abd, ek, L, cbW, aA, buk, GC, cbV, V, Kx, di, hb, WY, nX, aT, aaw, bK, an, cbX, af, eu, ak, aaE, aaO, t, xw, f, wA, dS, ck, axS, c, xB, AC, aen, ha, vr, eq, fu, xt, ot, cbH, sO, Dq, abg, abP, oj, Sb, cx, nJ, lx, gl, bOW, aF, Z, dr, GB, dR, bF, aqM, dJ, aaG, cbR, Kj, Gp, xg); #c1(MAPT) c2(NP_001116539) c3(microtubule-associated protein tau isoform 5) c4(43926) c5(43927, 43928, 43929, 43930, 43931, 43932, 43933, 43934, 43935, 43936, 43937, 43938, 43939, 43940) c6( ) c7(T, fR, bUk, e, Vd, aPl, aR, x, Gt, Fo, aU, K, H, li, aW, dg, cm, bb, jV, cbT, U, k, R, m, act, bmE, cZ, dt, dD, Ol, aw, nA, PJ, Mo, Hx, no, bl, cbU, dd, acM, aWO, dB, cbS, abd, ek, L, cbW, aA, buk, GC, cbV, V, Kx, di, hb, WY, nX, aT, aaw, bK, an, cbX, af, eu, ak, aaE, aaO, t, xw, f, wA, dS, ck, axS, c, xB, AC, aen, ha, vr, eq, fu, xt, ot, cbH, sO, Dq, abg, abP, oj, Sb, cx, nJ, lx, gl, bOW, aF, Z, dr, GB, dR, bF, aqM, dJ, aaG, cbR, Kj, Gp, xg); #c1(MAPT) c2(NP_001190180) c3(microtubule-associated protein tau isoform 7) c4(80312, 80313) c5(80314, 80315, 80316, 80317, 80318, 80319, 80320) c6( ) c7(T, fR, buk, e, Vd, aPl, aR, x, Gt, Fo, aU, K, H, li, aW, dg, cm, bb, jV, cbT, U, k, R, m, act, bmE, cZ, dt, dD, Ol, aw, nA, PJ, Mo, Hx, no, bl, cbU, dd, acM, aWO, dB, cbS, abd, ek, L, cbW, aA, buk, GC, cbV, V, Kx, di, hb, WY, nX, aT, aaw, bk, an, cbX, af, eu, ak, aaE, aaO, t, xw, f, wA, dS, ck, axS, c, xB, AC, aen, ha, vr, eq, fu, xt, ot, cbH, sO, Dq, abg, abP, oj, Sb, cx, nJ, lx, gl, bOW, aF, Z, dr, GB, dR, bF, aqM, dJ, aaG, cbR, Kj, Gp, xg); #c1(MAPT) c2(NP_001190181) c3(microtubule-associated protein tau isoform 8) c4(80321, 80322) c5(80323, 80324, 80325, 80326, 80327, 80328, 80329, 80330, 80331, 80332) c6( ) c7(T, fR, bUk, e, Vd, aPl, aR, x, Gt, Fo, aU, K, H, li, aW, dg, cm, bb, jV, cbT, U, k, R, m, act, bmE, cZ, dt, dD, Ol, aw, nA, PJ, Mo, Hx, no, bl, cbU, dd, acM, aWO, dB, cbS, abd, ek, L, cbW, aA, buk, GC, cbV, V, Kx, di, hb, WY, nX, aT, aaw, bK, an, cbX, af, eu, ak, aaE, aaD, t, xw, f, wA, dS, ck, axS, c, xB, AC, aen, ha, vr, eq, fu, xt, ot, cbH, sO, Dq, abg, abP, oj, Sb, cx, nJ, lx, gl, bOW, aF, Z, dr, GB, dR, bF, aqM, dJ, aaG, cbR, Kj, Gp, xg); #c1(MAPT) c2(NP_005901) c3(microtubule-associated protein tau isoform 2) c4(160224) c5(160225, 160226, 160227, 160228, 160229, 160230, 160231, 160232, 160233, 160234) c6( ) c7(T, fR, bUk, e, Vd, aPl, aR, x, Gt, Fo, aU, K, H, li, aW, dg, cm, bb, jV, cbT, U, k, R, m, act, bmE, cZ, dt, dD, Ol, aw, nA, PJ, Mo, Hx, no, bl, cbU, dd, acM, aWO, dB, cbS, abd, ek, L, cbW, aA, buk, GC, cbV, V, Kx, di, hb, WY, nX, aT, aaw, bK, an, cbX, af, eu, ak, aaE, aaD, t, xw, f, wA, dS, ck, axS, c, xB, AC, aen, ha, vr, eq, fu, xt, ot, cbH, sO, Dq, abg, abP, oj, Sb, cx, nJ, lx, gl, bOW, aF, Z, dr, GB, dR, bF, aqM, dJ, aaG, cbR, Kj, Gp, xg); #c1(MAPT) c2(NP_058518) c3(microtubule-associated protein tau isoform 3) c4(192750) c5(192751, 192752, 192753, 192754, 192755, 192756, 192757, 192758, 192759, 192760, 192761, 192762, 192763, 192764) c6( ) c7(T, fR, bUk, e, Vd, aPl, aR, x, Gt, Fo, aU, K, H, li, aW, dg, cm, bb, jV, cbT, U, k, R, m, act, bmE, cZ, dt, dD, Ol, aw, nA, PJ, Mo, Hx, no, bl, cbU, dd, acM, aWO, dB, cbS, abd, ek, L, cbW, aA, buk, GC, cbV, V, Kx, di, hb, WY, nX, aT, aaw, bK, an, cbX, af, eu, ak, aaE, aaD, t, xw, f, wA, dS, ck, axS, c, xB, AC, aen, ha, vr, eq, fu, xt, ot, cbH, sO, Dq, abg, abP, oj, Sb, cx, nJ, lx, gl, bOW, aF, Z, dr, GB, dR, bF, aqM, dJ, aaG, cbR, Kj, Gp, xg); #c1(MAPT) c2(NP_058519) c3(microtubule-associated protein tau isoform 1) c4(192765, 192766) c5(192767, 192768, 192769, 192770, 192771, 192772, 192773, 192774, 192775, 192776, 192777, 192778) c6( ) c7(T, fR, bUk, e, Vd, aPl, aR, x, Gt, Fo, aU, K, H, li, aW, dg, cm, bb, jV, cbT, U, k, R, m, act, bmE, cZ, dt, dD, Ol, aw, nA, PJ, Mo, Hx, no, bl, cbU, dd, acM, aWO, dB, cbS, abd, ek, L, cbW, aA, buk, GC, cbV, V, Kx, di, hb, WY, nX, aT, aaw, bK, an, cbX, af, eu, ak, aaE, aaD, t, xw, f, wA, dS, ck, axS, c, xB, AC, aen, ha, vr, eq, fu, xt, ot, cbH, sO, Dq, abg, abP, oj, Sb, cx, nJ, lx, gl, bOW, aF, Z, dr, GB, dR, bF, aqM, dJ, aaG, cbR, Kj, Gp, xg); #c1(MAPT) c2(NP_058525) c3(microtubule-associated protein tau isoform 4) c4(192786) c5(192787, 192788, 192789, 192790, 192791, 192792, 192793, 192794, 192795, 192796, 192797, 192798, 192799, 192800, 192801, 192802, 192803, 192804, 192805, 192806, 192807, 192808, 192809, 192810, 192811, 192812, 192813, 192814, 192815, 192816, 192817) c6( ) c7(T, fR, bUk, e, Vd, aPl, aR, x, Gt, Fo, aU, K, H, li, aW, dg, cm, bb, jV, cbT, U, k, R, m, act, bmE, cZ, dt, dD, Ol, aw, nA, PJ, Mo, Hx, no, bl, cbU, dd, acM, aWO, dB, cbS, abd, ek, L, cbW, aA, buk, GC, cbV, V, Kx, di, hb, WY, nX, aT, aaw, bk, an, cbX, af, eu, ak, aaE, aaD, t, xw, f, wA, dS, ck, axS, c, xB, AC, aen, ha, vr, eq, fu, xt, ot, cbH, sO, Dq, abg, abP, oj, Sb, cx, nJ, lx, gl, bOW, aF, Z, dr, GB, dR, bF, aqM, dJ, aaG, cbR, Kj, Gp, xg); #c1(MAPT) c2(XP_005257422) c3(microtubule-associated protein tau isoform X5) c4(275105) c5( ) c6( ) c7(T, FR, bUk, e, Vd, aPl, aR, x, Gt, Fo, aU, K, H, li, aW, dg, cm, bb, jV, cbT, U, k, R, m, act, bmE, cZ, dt, dD, Ol, aw, nA, PJ, Mo, Hx, no, bl, cbU, dd, acM, aWO, dB, cbS, abd, ek, L, cbW, aA, buk, GC, cbV, V, Kx, di, hb, WY, nX, aT, aaw, bK, an, cbX, af, eu, ak, aaE, aaD, t, xw, f, wA, dS, ck, axS, c, xB, AC, aen, ha, vr, eq, fu, xt, ot, cbH, sO, Dq, abg, abP, oj, Sb, cx, nJ, lx, gl, bOW, aF, Z, dr, GB, dR, bF, aqM, dJ, aaG, cbR, Kj, Gp, xg); #c1(MARC1) c2(NP_073583) c3(mitochondrial amidoxime-reducing component 1 precursor) c4(212973) c5(212974, 212975, 212976, 212977, 212978, 212979) c6( ) c7(qb, cd, xa); #c1(MARC2) c2(XP_011507986) c3(mitochondrial amidoxime reducing component 2 isoform X3) c4( ) c5(298423, 298424, 298425, 298426, 298427, 298428) c6( ) c7(ant, xa, abV, la); #c1(MARCH1) c2(NP_001159845) c3(E3 ubiquitin-protein ligase MARCH1 isoform 1) c4(64830, 64831) c5(64832, 64833, 64834, 64835, 64836, 64837, 64838, 64839, 64840, 64841, 64842, 64843, 64844, 64845, 64846, 64847) c6( ) c7(bL, ac, avm, aw, fe, m, t); #c1(MARCH1) c2(NP_060393) c3(E3 ubiquitin-protein ligase MARCH1 isoform 2) c4(195798) c5(195799, 195800, 195801, 195802, 195803, 195804) c6( ) c7(bL, ac, avm, aw, fe, m, t); #c1(MARCH2) c2(NP_001005415) c3(E3 ubiquitin-protein ligase MARCH2 isoform 1) c4(21667) c5(21668, 21669, 21670, 21671, 21672, 21673, 21674, 21675, 21676, 21677) c6( ) c7(T); #c1(MARCH2) c2(NP_001005416) c3(E3 ubiquitin-protein ligase MARCH2 isoform 2) c4(21678) c5(21679, 21680, 21681, 21682, 21683, 21684) c6( ) c7(T); #c1(MARCH5) c2(NP_060294) c3(E3 ubiquitin-protein ligase MARCH5) c4(195238) c5(195239, 195240, 195241, 195242, 195243, 195244) c6( ) c7(T, fq, bK); #c1(MARCH6) c2(NP_005876) c3(E3 ubiquitin-protein ligase MARCH6 isoform 1) c4(160067) c5(160068, 160069, 160070, 160071, 160072, 160073) c6( ) c7(aF, fq, ei, aA); #c1(MARCH7) c2(NP_001269735) c3(E3 ubiquitin-protein ligase MARCH7 isoform b) c4(102734) c5(102735, 102736, 102737, 102738, 102739, 102740, 102741, 102742, 102743, 102744, 102745, 102746) c6( ) c7(ck, ey); #c1(MARCH7) c2(NP_073737) c3(E3 ubiquitin-protein ligase MARCH7 isoform a) c4(213331, 213332) c5(213333, 213334, 213335, 213336, 213337, 213338, 213339, 213340, 213341, 213342) c6( ) c7(ck, ey); #c1(MARCH8) c2(NP_001002266) c3(E3 ubiquitin-protein ligase MARCH8 isoform a) c4(19512, 19513) c5(19514, 19515, 19516, 19517, 19518, 19519, 19520, 19521, 19522, 19523, 19524, 19525, 19526, 19527, 19528, 19529, 19530, 19531, 19532, 19533, 19534, 19535, 19536) c6( ) c7(bL, x, T, aE, bE, gl, ht, hL, cj, bB, fr, k, cl, iK, B); #c1(MARCH8) c2(NP_001269795) c3(E3 ubiquitin-protein ligase MARCH8 isoform b) c4(102858) c5(102859, 102860, 102861, 102862, 102863, 102864) c6( ) c7(bL, x, T, aE, bE, ql, ht, hL, cj, bB, fr, k, cl, iK, B); #c1(MARCKS) c2(NP_002347) c3(myristoylated alanine-rich C-kinase substrate) c4(127697, 127698) c5(127699, 127700, 127701, 127702, 127703, 127704) c6( ) c7(bM, T, al, bS, ct, ci, q, eA, jz, qK, aT, h, ey, K, nJ, d, aW, Y, r, nY, aq, g, Z, cm, iR, bz, bw, lU, gk, bE, ei, hE, B, ao, vi); #c1(MARC0) c2(NP_006761) c3(macrophage receptor MARC0) c4(167812, 167813) c5(167814, 167815, 167816, 167817, 167818, 167819, 167820) c6( ) c7(oO, ac, ig, ld, cr, azm, eE, y); #c1(MARC0) c2(XP_011510385) c3(macrophage receptor MARCO isoform X2) c4( ) c5(300913, 300914, 300915, 300916, 300917, 300918) c6( ) c7(oO, ac, ig, ld, cr, azm, eE, y); #c1(MARK1) c2(NP_001273055) c3(serine/threonine-protein kinase MARK1 isoform 3) c4(104267) c5(104268, 104269, 104270, 104271, 104272, 104273) c6( ) c7(T, nM, aA, azm, MC, fq, bN); #c1(MARK1) c2(NP_001273057) c3(serine/threonine-protein kinase MARK1 isoform 4) c4(104274) c5(104275, 104276, 104277, 104278, 104279, 104280) c6( ) c7(T, nM, aA, azm, MC, fq, bN); #c1(MARK1) c2(NP_061120) c3(serine/threonine-protein kinase MARK1 isoform 2) c4(199225) c5(199226, 199227, 199228, 199229, 199230, 199231) c6( ) c7(T, nM, aA, azm, MC, fq, bN); #c1(MARK2) c2(NP_001034558) c3(serine/threonine-protein kinase MARK2 isoform d) c4(31767, 31768) c5(31769, 31770, 31771, 31772, 31773, 31774, 31775, 31776, 31777, 31778, 31779, 31780, 31781, 31782, 31783, 31784, 31785, 31786, 31787, 31788, 31789, 31790, 31791, 31792, 31793, 31794, 31795, 31796, 31797) c6( ) c7(bM, T, aC, yS, if, fd, bB, ci, bv, aE, K, cp, aa, aW, bWB, g, Qt, eM, iT, m, vl, fr, nA, ai, Hx, jz, kH, h, OV, tl, at, fL, B, bL, fG, jx, aA, j, q, anj, hb, qK, bUn, aT, cy, an, ae, af, Y, r, lo, en, t, f, iK, ck, Qi, kP, fD, bt, ao, Kd, nZ, ha, jh, wV, hF, mC, ad, F, nd, d, ff, eZ, aF, bz, dr, qa, dR, LB, ek, bE, uR, ei, MC, y, X); #c1(MARK2) c2(NP_001156768) c3(serine/threonine-protein kinase MARK2 isoform e) c4(61931) c5(61932, 61933, 61934, 61935, 61936, 61937) c6( ) c7(bM, T, aC, yS, if, fd, bB, ci, bv, aE, K, cp, aa, aW, bWB, g, Qt, eM, iT, m, vl, fr, nA, ai, Hx, jz, kH, h, OV, tl, at, fL, B, bL, fG, jx, aA, j, q, anj, hb, qK, bUn, aT, cy, an, ae, af, Y, r, lo, en, t, f, iK, ck, Qi, kP, fD, bt, ao, Kd, nZ, ha, jh, wV, hF, mC, ad, F, nd, d, ff, eZ, aF, bz, dr, qa, dR, LB, ek, bE, UR, ei, MC, y, X); #c1(MARK2) c2(NP_001156769) c3(serine/threonine-protein kinase MARK2 isoform f) c4(61938, 61939) c5(61940, 61941, 61942, 61943, 61944, 61945, 61946, 61947, 61948, 61949, 61950, 61951, 61952, 61953, 61954, 61955, 61956, 61957, 61958, 61959, 61960, 61961, 61962, 61963, 61964, 61965, 61966, 61967, 61968) c6( ) c7(bM, T, aC, yS, if, fd, bB, ci, bv, aE, K, cp, aa, aW, bWB, g, Qt, eM, iT, m, vl, fr, nA, ai, Hx, jz, kH, h, OV, tl, at, fL, B, bL, fG, jx, aA, j, q, anj, hb, qK, bUn, aT, cy, an, ae, af, Y, r, lo, en, t, f, iK, ck, Qi, kP, fD, bt, ao, Kd, nZ, ha, jh, wV, hF, mC, ad, F, nd, d, ff, eZ, aF, bz, dr, qa, dR, LB, ek, bE, uR, ei, MC, y, X); #c1(MARK2) c2(NP_004945) c3(serine/threonine-protein kinase MARK2 isoform c) c4(151382, 151383, 151384) c5(151385, 151386, 151387, 151388, 151389, 151390, 151391, 151392, 151393, 151394, 151395, 151396, 151397) c6( ) c7(bM, T, aC, yS, if, fd, bB, ci, bv, aE, K, cp, aa, aW, bWB, g, Qt, eM, iT, m, vl, fr, nA, ai, Hx, jz, kH, h, OV, tl, at, fL, B, bL, fG, jx, aA, j, q, anj, hb, qK, bUn, aT, cy, an, ae, af, Y, r, lo, en, t, f, iK, ck, Qi, kP, fD, bt, ao, Kd, nZ, ha, jh, wV, hF, mC, ad, F, nd, d, ff, eZ, aF, bz, dr, qa, dR, LB, ek, bE, uR, ei, MC, y, X); #c1(MARK2) c2(XP_011543091) c3(serine/threonine-protein kinase MARK2 isoform X1) c4( ) c5(341927) c6( ) c7(bM, T, aC, yS, if, fd, bB, ci, bv, aE, K, cp, aa, aW, bWB, g, Qt, eM, iT, m, vl, fr, nA, ai, Hx, jz, kH, h, OV, tl, at, fL, B, bL, fG, jx, aA, j, q, anj, hb, qK, bUn, aT, cy, an, ae, af, Y, r, lo, en, t, f, iK, ck, Qi, kP, fD, bt, ao, Kd, nZ, ha, jh, wV, hF, mC, ad, F, nd, d, ff, eZ, aF, bz, dr, qa, dR, LB, ek, bE, uR, ei, MC, y, X); #c1(MARK2) c2(XP_011543092) c3(serine/threonine-protein kinase MARK2 isoform X2) c4(341928) c5( ) c6( ) c7(bM, T, aC, yS, if, fd, bB, ci, bv, aE, K, cp, aa, aW, bWB, g, Qt, eM, iT, m, vl, fr, nA, ai, Hx, jz, kH, h, OV, tl, at, fL, B, bL, fG, jx, aA, j, q, anj, hb, qK, bUn, aT, cy, an, ae, af, Y, r, lo, en, t, f, iK, ck, Qi, kP, fD, bt, ao, Kd, nZ, ha, jh, wV, hF, mC, ad, F, nd, d, ff, eZ, aF, bz, dr, qa, dR, LB, ek, bE, uR, ei, MC, y, X); #c1(MARK2) c2(XP_011543093) c3(serine/threonine-protein kinase MARK2 isoform X3) c4(341929) c5(341930, 341931, 341932, 341933, 341934, 341935) c6( ) c7(bM, T, aC, yS, if, fd, bB, ci, bv, aE, K, cp, aa, aW, bWB, g, Qt, eM, iT, m, vl, fr, nA, ai, Hx, jz, kH, h, OV, tl, at, fL, B, bL, fG, jx, aA, j, q, anj, hb, qK, bUn, aT, cy, an, ae, af, Y, r, lo, en, t, f, iK, ck, Qi, kP, fD, bt, ao, Kd, nZ, ha, jh, wV, hF, mC, ad, F, nd, d, ff, eZ, aF, bz, dr, qa, dR, LB, ek, bE, uR, ei, MC, y, X); #c1(MARK2) c2(XP_011543094) c3(serine/threonine-protein kinase MARK2 isoform X4) c4(341936) c5( ) c6( ) c7(bM, T, aC, yS, if, fd, bB, ci, bv, aE, K, cp, aa, aW, bWB, g, Qt, eM, iT, m, vl, fr, nA, ai, Hx, jz, kH, h, OV, tl, at, fL, B, bL, fG, jx, aA, j, q, anj, hb, qK, bUn, aT, cy, an, ae, af, Y, r, lo, en, t, f, iK, ck, Qi, kP, fD, bt, ao, Kd, nZ, ha, jh, wV, hF, mC, ad, F, nd, d, ff, eZ, aF, bz, dr, qa, dR, LB, ek, bE, uR, ei, MC, y, X); #c1(MARK2) c2(XP_011543095) c3(serine/threonine-protein kinase MARK2 isoform X5) c4(341937) c5( ) c6( ) c7(bM, T, aC, yS, if, fd, bB, ci, bv, aE, K, cp, aa, aW, bWB, g, Qt, eM, iT, m, vl, fr, nA, ai, Hx, jz, kH, h, OV, tl, at, fL, B, bL, fG, jx, aA, j, q, anj, hb, qK, bUn, aT, cy, an, ae, af, Y, r, lo, en, t, f, iK, ck, Qi, kP, fD, bt, ao, Kd, nZ, ha, jh, wV, hF, mC, ad, F, nd, d, ff, eZ, aF, bz, dr, qa, dR, LB, ek, bE, uR, ei, MC, y, X); #c1(MARK2) c2(XP_011543098) c3(serine/threonine-protein kinase MARK2 isoform X8) c4(341938) c5(341939, 341940, 341941, 341942, 341943, 341944, 341945, 341946, 341947) c6( ) c7(bM, T, aC, yS, if, fd, bB, ci, bv, aE, K, cp, aa, aW, bWB, g, Qt, eM, iT, m, vl, fr, nA, ai, Hx, jz, kH, h, OV, tl, at, fL, B, bL, fG, jx, aA, j, q, anj, hb, qK, bUn, aT, cy, an, ae, af, Y, r, lo, en, t, f, iK, ck, Qi, kP, fD, bt, ao, Kd, nZ, ha, jh, wV, hF, mC, ad, F, nd, d, ff, eZ, aF, bz, dr, qa, dR, LB, ek, bE, uR, ei, MC, y, X); #c1(MARK2) c2(XP_011543099) c3(serine/threonine-protein kinase MARK2 isoform X9) c4(341948, 341949) c5( ) c6( ) c7(bM, T, aC, yS, if, fd, bB, ci, bv, aE, K, cp, aa, aW, bWB, g, Qt, eM, iT, m, vl, fr, nA, ai, Hx, jz, kH, h, OV, tl, at, fL, B, bL, fG, jx, aA, j, q, anj, hb, qK, bUn, aT, cy, an, ae, af, Y, r, lo, en, t, f, iK, ck, Qi, kP, fD, bt, ao, Kd, nZ, ha, jh, wV, hF, mC, ad, F, nd, d, ff, eZ, aF, bz, dr, qa, dR, LB, ek, bE, uR, ei, MC, y, X); #c1(MARK2) c2(XP_011543100) c3(serine/threonine-protein kinase MARK2 isoform X10) c4( ) c5(341950, 341951, 341952, 341953, 341954, 341955) c6( ) c7(bM, T, aC, yS, if, fd, bB, ci, bv, aE, K, cp, aa, aW, bWB, g, Qt, eM, iT, m, vl, fr, nA, ai, Hx, jz, kH, h, OV, tl, at, fL, B, bL, fG, jx, aA, j, q, anj, hb, qK, bUn, aT, cy, an, ae, af, Y, r, lo, en, t, f, iK, ck, Qi, kP, fD, bt, ao, Kd, nZ, ha, jh, wV, hF, mC, ad, F, nd, d, ff, eZ, aF, bz, dr, qa, dR, LB, ek, bE, uR, ei, MC, y, X); #c1(MARK3) c2(NP_001122390) c3(MAP/microtubule affinity-regulating kinase 3 isoform a) c4(46608) c5(46609, 46610, 46611, 46612, 46613, 46614) c6( ) c7(kD, D, v, bA); #c1(MARK3) c2(NP_001122391) c3(MAP/microtubule affinity-regulating kinase 3 isoform b) c4(46615, 46616) c5(46617, 46618, 46619, 46620, 46621, 46622, 46623, 46624, 46625, 46626, 46627, 46628) c6( ) c7(kD, D, v, bA); #c1(MARK3) c2(NP_001122392) c3(MAP/microtubule affinity-regulating kinase 3 isoform d) c4(46629) c5(46630, 46631, 46632, 46633, 46634, 46635) c6( ) c7(kD, D, v, bA); #c1(MARK3) c2(NP_001122393) c3(MAP/microtubule affinity-regulating kinase 3 isoform e) c4(46636) c5(46637, 46638, 46639, 46640, 46641, 46642) c6( ) c7(kD, D, v, bA); #c1(MARK3) c2(NP_002367) c3(MAP/microtubule affinity-regulating kinase 3 isoform c) c4(127803, 127804) c5(127805, 127806, 127807, 127808, 127809, 127810, 127811, 127812, 127813, 127814, 127815, 127816) c6( ) c7(kD, D, v, bA); #c1(MARK3) c2(XP_005267699) c3(MAP/microtubule affinity-regulating kinase 3 isoform X3) c4(281222) c5(281223, 281224, 281225, 281226, 281227, 281228) c6( ) c7(kD, D, v, bA); #c1(MARK4) c2(NP_001186796) c3(MAP/microtubule affinity-regulating kinase 4 isoform 1) c4(78612) c5(78613, 78614, 78615, 78616, 78617, 78618) c6( ) c7(d, eA, af, MC); #c1(MARK4) c2(NP_113605) c3(MAP/microtubule affinity-regulating kinase 4 isoform 2) c4(221674, 221675) c5(221676, 221677, 221678, 221679, 221680, 221681, 221682, 221683, 221684, 221685, 221686, 221687, 221688) c6( ) c7(d, eA, af, MC); #c1(MARK4) c2(XP_006723370) c3(MAP/microtubule affinity-regulating kinase 4 isoform X1) c4(295179) c5(295180, 295181, 295182, 295183, 295184, 295185) c6( ) c7(d, eA, af, MC); #c1(MARS) c2(NP_004981) c3(methionine-tRNA ligase, cytoplasmic) c4(151577, 151578, 151579) c5(151580, 151581, 151582, 151583, 151584, 151585, 151586, 151587, 151588, 151589, 151590, 151591) c6( ) c7(cbY, aS, ad); #c1(MARS) c2(XP_011536655) c3(methionine-tRNA ligase, cytoplasmic isoform X2) c4( ) c5(334150, 334151, 334152, 334153, 334154, 334155) c6( ) c7(cbY, aS, ad); #c1(MARS2) c2(NP_612404) c3(methionine-tRNA ligase, mitochondrial precursor) c4(236399, 236400) c5(236401, 236402, 236403, 236404, 236405, 236406, 236407, 236408, 236409, 236410, 236411, 236412, 236413) c6( ) c7(cbZ); #c1(MARVELD1) c2(NP_113672) c3(MARVEL domain-containing protein 1) c4(222049) c5(222050, 222051, 222052, 222053, 222054, 222055) c6( ) c7(aW, KO, B, aT); #c1(MARVELD2) c2(NP_001231663) c3(MARVEL domain-containing protein 2 isoform 2) c4(87415) c5(87416, 87417, 87418, 87419, 87420, 87421, 87422, 87423, 87424, 87425, 87426, 87427, 87428, 87429, 87430, 87431, 87432, 87433) c6( ) c7(Bh, cca, Tp); #c1(MARVELD2) c2(XP_005248503) c3(MARVEL domain-containing protein 2 isoform X1) c4(268436, 268437, 268438, 268439, 268440) c5(268441, 268442, 268443, 268444, 268445, 268446, 268447, 268448, 268449, 268450, 268451, 268452, 268453, 268454, 268455, 268456, 268457, 268458) c6( ) c7(Bh, cca, Tp); #c1(MARVELD2) c2(XP_005276817) c3(MARVEL domain-containing protein 2 isoform X2) c4( ) c5(285735) c6( ) c7(Bh, cca, Tp); #c1(MARVELD3) c2(NP_001017967) c3(MARVEL domain-containing protein 3 isoform 1) c4(26684) c5(26685, 26686, 26687, 26688, 26689, 26690) c6( ) c7(T, aE, g); #c1(MARVELD3) c2(NP_001258258) c3(MARVEL domain-containing protein 3 isoform 3) c4( ) c5(96010) c6( ) c7(T, aE, g); #c1(MARVELD3) c2(NP_443090) c3(MARVEL domain-containing protein 3 isoform 2) c4(229342) c5(229343, 229344, 229345, 229346, 229347, 229348) c6( ) c7(T, aE, g); #c1(MAS1) c2(NP_002368) c3(proto-oncogene Mas) c4(127817) c5(127818, 127819, 127820, (27821, 127822, 127823) c6( ) c7(bL, bM, T, bcb, e, cz, v, C, cd, eA, ws, dl, vd, bq, aWf, eL, bc, A, je, xs, fl, si, Ku, as, dw, eE, wU, cT, iM, mx, HB, aS, pw, dy, bC, us, vQ); #c1(MAS1L) c2(NP_443199) c3(mas-related G-protein coupled receptor MRG) c4(229829) c5(229830, 229831, 229832, 229833, 229834, 229835, 229836, 229837, 229838, 229839, 229840, 229841, 229842, 229843, 229844, 229845, 229846, 229847, 229848, 229849, 229850, 229851, 229852, 229853, 229854, 229855, 229856) c6( ) c7(aj, J, eZ); #c1(MASP1) c2(NP_001027019) c3(mannan-binding lectin serine protease 1 isoform 3 precursor) c4(30089) c5(30090, 30091, 30092, 30093, 30094, 30095, 30096, 30097) c6( ) c7(T, jx, aTH, aTJ, aj, V, ai, q, J, ad, dL, ae, af, aTl, ff, Az, hl, r, en, W, Qu, WP, at); #c1(MASP1) c2(NP_001870) c3(mannan-binding lectin serine protease 1 isoform 1 precursor) c4(121850) c5(121851, 121852, 121853, 121854, 121855, 121856, 121857, 121858) c6( ) c7(T, jx, aTH, aTJ, aj, V, ai, q, J, ad, dL, ae, af, aTl, ff, Az, hl, r, en, W, Qu, WP, at); #c1(MASP1) c2(NP 624302) c3(mannan-binding lectin serine protease 1 isoform 2 precursor) c4(238143) c5(238144, 238145, 238146, 238147, 238148, 238149, 238150, 238151, 238152) c6( ) c7(T, jx, aTH, aTJ, aj, V, ai, q, J, ad, dL, ae, af, aTl, ff, Az, hl, r, en, W, Qu, WP, at); #c1(MASP1) c2(XP_006713763) c3(mannan-binding lectin serine protease 1 isoform X3) c4( ) c5(287314, 287315, 287316, 287317, 287318, 287319, 287320, 287321, 287322, 287323, 287324, 287325, 287326, 287327, 287328, 287329, 287330, 287331, 287332, 287333, 287334, 287335) c6(287336) c7(T, jx, aTH, aTJ, aj, V, ai, q, J, ad, dL, ae, af, aTl, ff, Az, hl, r, en, W, Qu, WP, at); #c1(MASP1) c2(XP_011511291) c3(mannan-binding lectin serine protease 1 isoform X1) c4(301800, 301801, 301802) c5( ) c6( ) c7(T, jx, aTH, aTJ, aj, V, ai, q, J, ad, dL, ae, af, aTl, ff, Az, hl, r, en, W, Qu, WP, at); #c1(MAST2) c2(XP_011539364) c3(microtubule-associated serine/threonine-protein kinase 2 isoform X4) c4(337460) c5(337461, 337462, 337463, 337464, 337465, 337466) c6( ) c7(aW, d, aT); #c1(MAST2) c2(XP_011539370) c3(microtubule-associated serine/threonine-protein kinase 2 isoform X16) c4( ) c5(337467, 337468, 337469, 337470, 337471, 337472, 337473, 337474, 337475, 337476, 337477, 337478) c6( ) c7(aW, d, aT); #c1(MAST2) c2(XP_011539371) c3(microtubule-associated serine/threonine-protein kinase 2 isoform X17) c4( ) c5(337479, 337480, 337481, 337482, 337483, 337484) c6( ) c7(aW, d, aT); #c1(MAST4) c2(NP_001158136) c3(microtubule-associated serine/threonine-protein kinase 4 isoform c) c4(63162, 63163) c5(63164, 63165, 63166, 63167, 63168, 63169, 63170, 63171, 63172, 63173, 63174, 63175) c6( ) c7(bL); #c1(MAST4) c2(NP_001277155) c3(microtubule-associated serine/threonine-protein kinase 4 isoform d) c4(109179, 109180) c5(109181, 109182, 109183, 109184, 109185, 109186, 109187, 109188, 109189, 109190, 109191, 109192) c6( ) c7(bL); #c1(MAST4) c2(NP_001277156) c3(microtubule-associated serine/threonine-protein kinase 4 isoform e) c4(109193) c5(109194, 109195, 109196, 109197, 109198, 109199, 109200) c6( ) c7(bL); #c1(MAST4) c2(NP_055998) c3(microtubule-associated serine/threonine-protein kinase 4 isoform a) c4(185034) c5(185035, 185036, 185037, 185038, 185039, 185040) c6( ) c7(bL); #c1(MAST4) c2(NP_942123) c3(microtubule-associated serine/threonine-protein kinase 4 isoform b) c4(260473) c5(260474, 260475, 260476, 260477, 260478, 260479) c6( ) c7(bL); #c1(MAST4) c2(XP_006714669) c3(microtubule-associated serine/threonine-protein kinase 4 isoform X2) c4(287982, 287983) c5(287984, 287985, 287986, 287987, 287988, 287989, 287990, 287991, 287992, 287993, 287994, 287995) c6( ) c7(bL); #c1(MASTL) c2(NP_001165774) c3(serine/threonine-protein kinase greatwall isoform 1) c4(67892) c5(67893, 67894, 67895, 67896, 67897, 67898) c6( ) c7(DU, Wa); #c1(MASTL) c2(NP_001165775) c3(serine/threonine-protein kinase greatwall isoform 3) c4(67899) c5(67900, 67901, 67902, 67903, 67904, 67905) c6( ) c7(DU, Wa); #c1(MASTL) c2(NP_116233) c3(serine/threonine-protein kinase greatwall isoform 2) c4(225878) c5(225879, 225880, 225881, 225882, 225883, 225884) c6( ) c7(DU, Wa); #c1(MAT1A) c2(XP_005269899) c3(S-adenosylmethionine synthase isoform type-l isoform X1) c4(282978) c5(282979, 282980, 282981, 282982, 282983, 282984, 282985, 282986, 282987, 282988) c6( ) c7(bL, T, e, al, cz, ci, mF, ad, Na, aE, hb, rJ, aT, rN, rO, Nc, K, ZN, ae, rL, KC, MX, aW, fc, dq, aq, bFG, bFF, cm, iR, gk, mO, ac, Ip, aBO, rH, cP, B); #c1(MAT1A) c2(XP_005269900) c3(S-adenosylmethionine synthase isoform type-1 isoform X2) c4(282989) c5(282990, 282991, 282992, 282993, 282994, 282995) c6( ) c7(bL, T, e, al, cz, ci, mF, ad, Na, aE, hb, rJ, aT, rN, rO, Nc, K, ZN, ae, rL, KC, MX, aW, fc, dq, aq, bFG, bFF, cm, iR, gk, mO, ac, lp, aBO, rH, cP, B); #c1(MAT2A) c2(NP_005902) c3(S-adenosylmethionine synthase isoform type-2) c4(160235, 160236) c5(160237, 160238, 160239, 160240, 160241, 160242, 160243, 160244, 160245, 160246, 160247, 160248) c6( ) c7(Na, T, B, lp, bE, r, aT, bB, aq, Nc, ey, ci, fr, iR, cm, X, hX); #c1(MAT2B) c2(NP_037415) c3(methionine adenosyltransferase 2 subunit beta isoform 1) c4(175387, 175388, 175389) c5(175390, 175391, 175392, 175393, 175394, 175395, 175396, 175397, 175398, 175399, 175400, 175401) c6( ) c7(Na, uH, W, pN, lp, e, aw, cz, gZ, cy, cd, aj, aq, Nc, sl, cm, B, J); #c1(MAT2B) c2(NP_877725) c3(methionine adenosyltransferase 2 subunit beta isoform 2) c4(256949) c5(256950, 256951, 256952, 256953, 256954, 256955, 256956, 256957, 256958, 256959, 256960, 256961) c6( ) c7(Na, uH, W, pN, lp, e, aw, cz, gZ, cy, cd, aj, aq, Nc, sl, cm, B, J); #c1(MATK) c2(NP_002369) c3(megakaryocyte-associated tyrosine-protein kinase isoform b) c4(127824) c5(127825, 127826, 127827, 127828, 127829, 127830) c6( ) c7(ck, T, d, aE, dB, aW, jh, aT, v, K, as, cm, bz, X); #c1(MATK) c2(NP_64761 l) c3(megakaryocyte-associated tyrosine-protein kinase isoform c) c4(238804) c5( ) c6( ) c7(ck, T, d, aE, dB, aW, jh, aT, v, K, as, cm, bz, X); #c1(MATK) c2(XP_011526320) c3(megakaryocyte-associated tyrosine-protein kinase isoform X1) c4(320620) c5(320621, 320622, 320623, 320624, 320625, 320626, 320627, 320628, 320629, 320630, 320631, 320632) c6( ) c7(ck, T, d, aE, dB, aW, jh, aT, v, K, as, cm, bz, X); #c1(MATN2) c2(NP_002371) c3(matrilin-2 isoform a precursor) c4(127831, 127832, 127833, 127834) c5(127835, 127836, 127837, 127838, 127839, 127840, 127841, 127842, 127843, 127844, 127845, 127846, 127847, 127848, 127849, 127850, 127851, 127852) c6( ) c7(Ew, B, as, Hp); #c1(MATN2) c2(NP_085072) c3(matrilin-2 isoform b precursor) c4(219256) c5(219257, 219258, 219259, 219260, 219261, 219262) c6( ) c7(Ew, B, as, Hp); #c1(MATN2) c2(XP_005250977) c3(matrilin-2 isoform X2) c4( ) c5(270717, 270718, 270719, 270720, 270721, 270722) c6( ) c7(Ew, B, as, Hp); #c1(MATN3) c2(NP_002372) c3(matrilin-3 precursor) c4(127853, 127854) c5(127855, 127856, 127857, 127858, 127859, 127860, 127861, 127862, 127863, 127864, 127865, 127866) c6( ) c7(Dg, kh, zw, rq, ccb, ru, ry, ln, rs, Dj, K, rv, ccc); #c1(MATR3) c2(NP_001181883) c3(matrin-3 isoform a) c4(74308, 74309, 74310, 74311, 74312, 74313, 74314, 74315) c5(74316, 74317, 74318, 74319, 74320, 74321, 74322, 74323, 74324, 74325, 74326, 74327, 74328, 74329, 74330, 74331, 74332, 74333, 74334, 74335, 74336, 74337, 74338, 74339, 74340, 74341, 74342, 74343, 74344, 74345, 74346, 74347, 74348, 74349, 74350, 74351, 74352, 74353, 74354, 74355, 74356, 74357, 74358, 74359, 74360, 74361, 74362, 74363, 74364, 74365, 74366, 74367, 74368, 74369, 74370, 74371, 74372, 74373, 74374, 74375, 74376) c6( ) c7(ccd, no, Ws, xs, iw, oj, at, iv, h, dl, bw, f, cce); #c1(MATR3) c2(NP_001181885) c3(matrin-3 isoform b) c4(74377, 74378) c5(74379, 74380, 74381, 74382, 74383, 74384, 74385, 74386) c6( ) c7(ccd, no, Ws, xs, iw, oj, at, iv, h, dl, bw, f, cce); #c1(MATR3) c2(NP_001269207) c3(matrin-3 isoform c) c4(100764) c5(100765, 100766, 100767, 100768, 100769, 100770, 100771, 100772, 100773, 100774, 100775, 100776, 100777, 100778) c6( ) c7(ccd, no, Ws, xs, iw, oj, at, iv, h, dl, bw, f, cce); #c1(MAU2) c2(NP_056144) c3(MAU2 chromatid cohesion factor homolog) c4(185938, 185939) c5(185940, 185941, 185942, 185943, 185944, 185945, 185946, 185947, 185948, 185949, 185950, 185951) c6( ) c7(T, Y, ff, aNL, en, qN, nz, iK, bz); #c1(MAU2) c2(XP_011526146) c3(MAU2 chromatid cohesion factor homolog isoform X5) c4( ) c5(320407, 320408, 320409, 320410, 320411, 320412) c6( ) c7(T, Y, ff, aNL, en, qN, nz, iK, bz); #c1(MAVS) c2(NP_001193420) c3(mitochondrial antiviral-signaling protein isoform 2) c4(82180) c5(82181, 82182, 82183, 82184, 82185, 82186) c6( ) c7(gV, T, bM, cch, Dw, fR, jx, aFc, V, mC, F, ccg, ds, ccf, cU, aT, DU, aW, Y, eZ, aiT, lo, CQ, pm, t, m, fl); #c1(MAVS) c2(NP_065797) c3(mitochondrial antiviral-signaling protein isoform 1) c4(205494, 205495, 205496) c5(205497, 205498, 205499, 205500, 205501, 205502, 205503, 205504, 205505, 205506, 205507, 205508, 205509, 205510, 205511, 205512, 205513, 205514) c6( ) c7(gV, T, bM, cch, Dw, fR, jx, aFc, V, mC, F, ccg, ds, ccf, cu, aT, DU, aW, Y, eZ, aiT, lo, CQ, pm, t, m, fL); #c1(MAX) c2(NP_002373) c3(protein max isoform a) c4(127867, 127868) c5(127869, 127870, 127871, 127872, 127873, 127874, 127875, 127876, 127877, 127878, 127879, 127880, 127881, 127882, 127883, 127884, 127885, 127886, 127887, 127888, 127889, 127890, 127891, 127892, 127893, 127894, 127895, 127896, 127897, 127898, 127899, 127900, 127901, 127902, 127903, 127904, 127905, 127906, 127907, 127908, 127909, 127910, 127911, 127912, 127913, 127914, 127915, 127916, 127917, 127918, 127919, 127920, 127921, 127922, 127923, 127924, 127925, 127926, 127927, 127928, 127929, 127930) c6( ) c7(jf, av, T, ft, pf, dO, anx, EQ, r, tv, tu, ei, g, cm, q); #c1(MAX) c2(NP_660087) c3(protein max isoform b) c4(240311) c5(240312, 240313, 240314, 240315, 240316, 240317) c6( ) c7(jf, av, T, ft, pf, dO, anx, EQ, r, tv, tu, ei, g, cm, q); #c1(MAX) c2(NP_660088) c3(protein max isoform c) c4(240318, 240319, 240320) c5(240321, 240322, 240323, 240324, 240325, 240326, 240327, 240328, 240329, 240330, 240331, 240332, 240333, 240334, 240335, 240336, 240337, 240338, 240339, 240340, 240341) c6( ) c7(jf, av, T, ft, pf, dO, anx, EQ, r, tv, tu, ei, g, cm, q); #c1(MAX) c2(NP_660089) c3(protein max isoform d) c4(240342) c5(240343, 240344, 240345, 240346, 240347, 240348) c6( ) c7(jf, av, T, ft, pf, dO, anx, EQ, r, tv, tu, ei, g, cm, q); #c1(MAX) c2(NP_932061) c3(protein max isoform f) c4(258726) c5(258727, 258728, 258729, 258730, 258731, 258732) c6( ) c7(jf, av, T, ft, pf, dO, anx, EQ, r, tv, tu, ei, g, cm, q); #c1(MAZ) c2(NP_001036004) c3(myc-associated zinc finger protein isoform 2) c4(33664) c5(33665, 33666, 33667, 33668, 33669, 33670) c6( ) c7(bxy, Ng, dD, d, jz, aW, ea, Pg, VB, aT, h, aj, eq, R, Lz, bt, ai, J, bN); #c1(MAZ) c2(NP_001263204) c3(myc-associated zinc finger protein isoform 3) c4(97027, 97028) c5(97029, 97030, 97031, 97032, 97033, 97034, 97035, 97036, 97037, 97038, 97039, 97040) c6( ) c7(bxy, Ng, dD, d, jz, aW, ea, Pg, VB, aT, h, aj, eq, R, Lz, bt, ai, J, bN); #c1(MAZ) c2(NP_001263205) c3(myc-associated zinc finger protein isoform 4) c4( ) c5(97041) c6( ) c7(bxy, Ng, dD, d, jz, aW, ea, Pg, VB, aT, h, aj, eq, R, Lz, bt, ai, J, bN); #c1(MAZ) c2(NP_002374) c3(myc-associated zinc finger protein isoform 1) c4(127931) c5(127932, 127933, 127934, 127935, 127936, 127937) c6( ) c7(bxy, Ng, dD, d, jz, aW, ea, Pg, VB, aT, h, aj, eq, R, Lz, bt, ai, J, bN); #c1(MB) c2(XP_005261662) c3(myoglobin isoform X1) c4(277525, 277526, 277527, 277528, 277529) c5(277530, 277531, 277532, 277533, 277534, 277535, 277536, 277537, 277538, 277539, 277540, 277541, 277542, 277543, 277544, 277545, 277546, 277547, 277548, 277549, 277550, 277551, 277552, 277553, 277554, 277555, 277556, 277557, 277558, 277559, 277560, 277561, 277562, 277563, 277564, 277565, 277566, 277567, 277568, 277569, 277570, 277571, 277572, 277573, 277574, 277575) c6( ) c7(T, Or, aw, j, ci, fr, pd, iJ, ip, bv, no, py, Bc, ry, oj, aT, K, aW, xs, uY, g, cm, Bu, bz, kC, eM, iA, aNJ, E, cci); #c1(MB21D1) c2(NP_612450) c3(cyclic GMP-AMP synthase) c4(236516) c5(236517, 236518, 236519, 236520, 236521, 236522) c6( ) c7(oo, gl); #c1(MB21D1) c2(XP_006715390) c3(cyclic GMP-AMP synthase isoform X1) c4(289498) c5(289499, 289500, 289501, 289502, 289503, 289504, 289505, 289506, 289507, 289508) c6( ) c7(oo, gl); #c1(MB21D2) c2(NP_848591) c3(protein MB21D2) c4(253909) c5(253910, 253911, 253912, 253913, 253914, 253915, 253916) c6( ) c7(fq); #c1(MBD1) c2(NP_001191065) c3(methyl-CpG-binding domain protein 1 isoform 6) c4(80901) c5(80902, 80903, 80904, 80905, 80906, 80907, 80908, 80909) c6( ) c7(T, aC, hy, qj, ci, hF, ad, av, aE, ae, BN, vs, Fq, Tp, g, bz, bN, Qi, ou, rR, nM, nB, bE, cj, m, ne, pC); #c1(MBD1) c2(NP_001191070) c3(methyl-CpG-binding domain protein 1 isoform 10) c4(80910) c5(80911, 80912, 80913, 80914, 80915, 80916) c6( ) c7(T, aC, hy, qj, ci, hF, ad, av, aE, ae, BN, vs, Fq, Tp, g, bz, bN, Qi, ou, rR, nM, nB, bE, cj, m, ne, pC); #c1(MBD1) c2(NP_001191071) c3(methyl-CpG-binding domain protein 1 isoform 11) c4(80917) c5(80918, 80919, 80920, 80921, 80922, 80923) c6( ) c7(T, aC, hy, qj, ci, hF, ad, av, aE, ae, BN, vs, Fq, Tp, g, bz, bN, Di, ou, rR, nM, nB, bE, cj, m, ne, pC); #c1(MBD1) c2(NP_001191080) c3(methyl-CpG-binding domain protein 1 isoform (3) c4(80973) c5(80974, 80975, 80976, 80977, 80978) c6( ) c7(T, aC, hy, qj, ci, hF, ad, av, aE, ae, BN, vs, Fq, Tp, g, bz, bN, Qi, ou, rR, nM, nB, bE, cj, m, ne, pC); #c1(MBD1) c2(NP_002375) c3(methyl-CpG-binding domain protein 1 isoform 4) c4(127938) c5(127939, 127940, 127941, 127942, 127943, 127944, 127945, 127946, 127947, 127948) c6( ) c7(T, aC, hy, qj, ci, hF, ad, av, aE, ae, BN, vs, Fq, Tp, g, bz, bN, Qi, ou, rR, nM, nB, bE, cj, m, ne, pC); #c1(MBD1) c2(NP_056669) c3(methyl-CpG-binding domain protein 1 isoform 3) c4(188119) c5(188120, 188121, 188122, 188123, 188124, 188125, 188126, 188127, 188128) c6( ) c7(T, aC, hy, qj, ci, hF, ad, av, aE, ae, BN, vs, Fq, Tp, g, bz, bN, Qi, ou, rR, nM, nB, bE, cj, m, ne, pC); #c1(MBD1) c2(NP_056670) c3(methyl-CpG-binding domain protein 1 isoform 2) c4(188129) c5(188130, 188131, 188132, 188133, 188134, 188135, 188136) c6( ) c7(T, aC, hy, qj, ci, hF, ad, av, aE, ae, BN, vs, Fq, Tp, g, bz, bN, Qi, ou, rR, nM, nB, bE, cj, m, ne, pC); #c1(MBD1) c2(NP_056723) c3(methyl-CpG-binding domain protein 1 isoform 5) c4(188137) c5(188138, 188139, 188140, 188141, 188142) c6( ) c7(T, aC, hy, qj, ci, hF, ad, av, aE, ae, BN, vs, Fq, Tp, g, bz, bN, Qi, ou, rR, nM, nB, bE, cj, m, ne, pC); #c1(MBD1) c2(XP_005258328) c3(methyl-CpG-binding domain protein 1 isoform X1) c4(275627) c5(275628, 275629, 275630, 275631, 275632, 275633, 275634, 275635, 275636, 275637, 275638, 275639) c6( ) c7(T, aC, hy, qj, ci, hF, ad, av, aE, ae, BN, vs, Fq, Tp, g, bz, bN, Di, ou, rR, nM, nB, bE, cj, m, ne, pC); #c1(MBD1) c2(XP_011524289) c3(methyl-CpG-binding domain protein 1 isoform X4) c4( ) c5(318781) c6( ) c7(T, aC, hy, qj, ci, hF, ad, av, aE, ae, BN, vs, Fq, Tp, g, bz, bN, Qi, ou, rR, nM, nB, bE, cj, m, ne, pC); #c1(MBD1) c2(XP_011524297) c3(methyl-CpG-binding domain protein 1 isoform X2) c4(318782, 318783) c5(318784, 318785, 318786, 318787, 318788, 318789, 318790, 318791, 318792, 318793, 318794) c6( ) c7(T, aC, hy, qj, ci, hF, ad, av, aE, ae, BN, vs, Fq, Tp, g, bz, bN, Qi, ou, rR, nM, nB, bE, cj, m, ne, pC); #c1(MBD1) c2(XP_011524300) c3(methyl-CpG-binding domain protein 1 isoform X12) c4( ) c5(318795) c6( ) c7(T, aC, hy, qj, ci, hF, ad, av, aE, ae, BN, vs, Fq, Tp, g, bz, bN, Qi, ou, rR, nM, nB, bE, cj, m, ne, pC); #c1(MBD1) c2(XP_011524303) c3(methyl-CpG-binding domain protein 1 isoform X15) c4( ) c5(318796, 318797) c6( ) c7(T, aC, hy, qj, ci, hF, ad, av, aE, ae, BN, vs, Fq, Tp, g, bz, bN, Qi, ou, rR, nM, nB, bE, cj, m, ne, pC); #c1(MBD1) c2(XP_011524308) c3(methyl-CpG-binding domain protein 1 isoform X3) c4(318798) c5(318799, 318800, 318801, 318802, 318803, 318804) c6( ) c7(T, aC, hy, qj, ci, hF, ad, av, aE, ae, BN, vs, Fq, Tp, g, bz, bN, Di, ou, rR, nM, nB, bE, cj, m, ne, pC); #c1(MBD2) c2(NP_003918) c3(methyl-CpG-binding domain protein 2 isoform 1) c4(142154) c5(142155, 142156, 142157, 142158, 142159, 142160) c6( ) c7(T, kh, id, iW, arQ, oA, aA, cz, bB, jh, dG, ci, hF, AO, iJ, q, ccj, mC, ad, hX, eA, bv, biS, aT, dL, jj, ae, eL, avl, aa, d, s, aNl, aW, eH, eZ, la, nf, r, aq, vs, kp, aF, g, eF, t, cm, bz, bcN, Hv, pb, iR, ck, bw, Bv, eM, dB, bE, eD, Qu, at, np, m, fl, bt, it, B); #c1(MBD2) c2(NP_056647) c3(methyl-CpG-binding domain protein 2 testis-specific isoform) c4(188088) c5(188089, 188090, 188091, 188092, 188093, 188094, 188095, 188096, 188097, 188098) c6( ) c7(T, kh, id, iW, arQ, oA, aA, cz, bB, jh, dG, ci, hF, AO, iJ, q, ccj, mC, ad, hX, eA, bv, biS, aT, dL, jj, ae, eL, avl, aa, d, s, aNl, aW, eH, eZ, la, nf, r, aq, vs, kp, aF, g, eF, t, cm, bz, bcN, Hv, pb, iR, ck, bw, Bv, eM, dB, bE, eD, Qu, at, np, m, fl, bt, it, B); #c1(MBD3) c2(NP_001268382) c3(methyl-CpG-binding domain protein 3 isoform 1) c4(99810) c5(99811, 99812, 99813, 99814, 99815, 99816, 99817, 99818) c6( ) c7(T, eM, aD, vs, qj, jj, bz, bN, pC); #c1(MBD3) c2(NP_001268383) c3(methyl-CpG-binding domain protein 3 isoform 2) c4(99819) c5(99820, 99821, 99822, 99823, 99824, 99825) c6( ) c7(T, eM, aD, vs, qj, jj, bz, bN, pC); #c1(MBD3L2) c2(NP_653215) c3(methyl-CpG-binding domain protein 3-like 2) c4(239115, 239116, 239117) c5(239118, 239119, 239120, 239121, 239122, 239123, 239124, 239125, 239126, 239127, 239128, 239129, 239130, 239131, 239132, 239133, 239134, 239135) c6( ) c7(aA); #c1(MBD4) c2(NP_001263199) c3(methyl-CpG-binding domain protein 4 isoform 2) c4(97013) c5(97014, 97015, 97016, 97017, 97018, 97019) c6( ) c7(T, aA, bB, V, q, ad, eA, av, aE, aT, dL, DY, ae, dx, jf, iF, s, aW, Y, eZ, r, aF, Fq, g, t, bz, pb, bN, eM, bE, gX, at, m, y, it, X); #c1(MBD4) c2(NP_001263202) c3(methyl-CpG-binding domain protein 4 isoform 5) c4(97020) c5(97021, 97022, 97023, 97024, 97025, 97026) c6( ) c7(T, aA, bB, V, q, ad, eA, av, aE, aT, dL, DY, ae, dx, jf, iF, s, aW, Y, eZ, r, aF, Fq, g, t, bz, pb, bN, eM, bE, gX, at, m, y, it, X); #c1(MBD4) c2(NP_003916) c3(methyl-CpG-binding domain protein 4 isoform 1) c4(142146) c5(142147, 142148, 142149, 142150, 142151, 142152, 142153) c6( ) c7(T, aA, bB, V, q, ad, eA, av, aE, aT, dL, DY, ae, dx, jf, iF, s, aW, Y, eZ, r, aF, Fq, g, t, bz, pb, bN, eM, bE, gX, at, m, y, it, X); #c1(MBD5) c2(NP_060798) c3(methyl-CpG-binding domain protein 5) c4(197891) c5(197892, 197893, 197894, 197895, 197896, 197897) c6( ) c7(cck, agd, ac, ha, amj, bK, nA, rz, B, bN, hq); #c1(MBD6) c2(NP_443129) c3(methyl-CpG-binding domain protein 6) c4(229565, 229566, 229567, 229568, 229569, 229570) c5( ) c6( ) c7(bN, bX); #c1(MBD6) c2(XP_005268677) c3(methyl-CpG-binding domain protein 6 isoform X2) c4( ) c5(282014, 282015, 282016, 282017, 282018, 282019, 282020, 282021, 282022, 282023, 282024, 282025, 282026, 282027, 282028, 282029, 282030, 282031, 282032, 282033, 282034, 282035, 282036, 282037, 282038, 282039, 282040, 282041, 282042, 282043, 282044, 282045) c6( ) c7(bN, bX); #c1(MBD6) c2(XP_011536153) c3(methyl-CpG-binding domain protein 6 isoform X1) c4( ) c5(333461, 333462, 333463, 333464, 333465, 333466, 333467, 333468, 333469, 333470, 333471, 333472, 333473) c6( ) c7(bN, bX); #c1(MBIP) c2(NP_00138363) c3(MAP3K12-binding inhibitory protein 1 isoform 2) c4(56376) c5(56377, 56378, 56379, 56380, 56381, 56382) c6( ) c7(aW, aT); #c1(MBIP) c2(NP_057670) c3(MAP3K12-binding inhibitory protein 1 isoform 1) c4(192270) c5(192271, 192272, 192273, 192274, 192275, 192276) c6( ) c7(aW, aT); #c1(MBL2) c2(XP_011538118) c3(mannose-binding protein C isoform X1) c4(335692) c5(335693, 335694, 335695, 335696, 335697, 335698) c6( ) c7(bol, oO, T, bM, fR, com, ea, if, e, al, eb, bvq, bB, cL, gA, qj, aoj, fd, fw, aR, aEl, wa, NF, bPE, AW, xy, x, ws, JM, dV, Ds, K, aaW, cco, zy, hH, bc, hm, aa, aW, az, sP, hl, mQ, kt, pD, pL, ve, akw, axp, uC, ccs, eE, y, fT, yl, anT, aFO, nB, dc, ki, bPC, eM, bTe, cj, uo, Zn, uJ, QF, k, m, R, aNn, eg, aFf, bsh, sr, ajy, dD, JZ, sQ, aw, fN, ez, xM, n, aj, aCZ, pM, qN, eh, zo, ai, eW, aDF, ke, aBy, b, no, afn, cU, lG, Ne, dL, DU, wb, ey, h, aDp, Nr, OV, mR, A, Cw, ge, fe, aCE, aq, po, ajz, cM, sE, xe, Zj, acD, Wt, adz, bMc, KT, ar, bkn, W, apO, NL, aZL, RJ, mg, axj, hs, uz, ut, aOL, eo, fl, aDE, at, bni, Pr, B, it, bL, Dw, ka, jv, pF, ach, AL, eR, oe, ccr, DV, jx, aA, cz, fF, hz, v, j, OY, C, bX, aBo, V, rd, q, J, o, eA, con, ga, aEE, aZX, aOj, bNQ, aWc, jZ, sl, qp, aWJ, aT, rS, cy, bsi, aAD, yb, an, af, eu, uA, blm, hM, oV, bsb, DN, lC, r, cct, afq, lo, aDm, afs, t, aMf, eL, lY, ck, iQ, sn, ac, and, hA, ig, AH, hr, auT, aYx, wR, CZ, cS, dy, JH, fD, gT, bt, ao, yv, gx, sY, Ve, Kd, aFe, gV, qb, aD, aPJ, Hc, uG, axd, iW, jh, vr, OR, wV, eq, jE, atK, Fc, mC, Uy, ad, hX, bnZ, hd, F, od, afm, ccq, ccu, ccv, ds, acu, oW, Os, hD, ccl, JK, lQ, bOu, vO, yF, sj, uQ, uX, aVZ, aFP, d, hG, cW, aZK, xs, wc, bHX, eZ, Mz, aFr, aF, hp, avG, aFd, ccp, iV, bz, ek, Nq, D, gk, Bv, zr, atM, bE, z, anY, afb, aED, wz, sG, aS, Lz, i, lf, ra, ne, hv, pC); #c1(MBNL1) c2(NP_066368) c3(muscleblind-like protein 1 isoform a) c4(207265) c5(207266, 207267, 207268, 207269, 207270, 207271) c6( ) c7(I, OV, u, oj, ey, bzq, dR); #c1(MBNL1) c2(NP_997175) c3(muscleblind-like protein 1 isoform b) c4(265075) c5(265076, 265077, 265078, 265079, 265080, 265081) c6( ) c7(I, OV, u, oj, ey, bzq, dR); #c1(MBNL1) c2(NP_997176) c3(muscleblind-like protein 1 isoform c) c4(265082) c5(265083, 265084, 265085, 265086, 265087, 265088, 265089, 265090, 265091, 265092) c6( ) c7(l, OV, u, oj, ey, bzq, dR); #c1(MBNL1) c2(NP_997177) c3(muscleblind-like protein 1 isoform d) c4(265093) c5(265094, 265095, 265096, 265097, 265098, 265099, 265100, 265101, 265102, 265103) c6( ) c7(I, OV, u, oj, ey, bzq, dR); #c1(MBNL1) c2(NP_997178) c3(muscleblind-like protein 1 isoform e) c4(265104) c5(265105, 265106, 265107, 265108, 265109, 265110) c6( ) c7(I, OV, u, oj, ey, bzq, dR); #c1(MBNL1) c2(NP_997179) c3(muscleblind-like protein 1 isoform f) c4(265111) c5(265112, 265113, 265114, 265115, 265116, 265117, 265118, 265119, 265120, 265121) c6( ) c7(I, OV, u, oj, ey, bzq, dR); #c1(MBNL1) c2(NP_997180) c3(muscleblind-like protein 1 isoform g) c4(265122) c5(265123, 265124, 265125, 265126, 265127, 265128, 265129, 265130, 265131, 265132) c6( ) c7(I, OV, u, oj, ey, bzq, dR); #c1(MBNL1) c2(XP_005247534) c3(muscleblind-like protein 1 isoform X19) c4( ) c5(267863) c6( ) c7(l, OV, u, oj, ey, bzq, dR); #c1(MBNL2) c2(NP_659002) c3(muscleblind-like protein 2 isoform 1) c4(239675) c5(239676, 239677, 239678, 239679, 239680, 239681) c6( ) c7(aj, lU, oj, dR); #c1(MBNL2) c2(NP_997187) c3(muscleblind-like protein 2 isoform 3) c4(265141) c5(265142, 265143, 265144, 265145, 265146, 265147, 265148, 265149, 265150, 265151, 265152, 265153, 265154, 265155, 265156, 265157) c6( ) c7(aj, lU, oj, dR); #c1(MBNL2) c2(XP_005254076) c3(muscleblind-like protein 2 isoform X3) c4(272868) c5( ) c6( ) c7(aj, lU, oj, dR); #c1(MBNL2) c2(XP_005254078) c3(muscleblind-like protein 2 isoform X4) c4(272869) c5(272870, 272871, 272872, 272873, 272874, 272875) c6( ) c7(aj, lU, oj, dR); #c1(MBNL2) c2(XP_011519347) c3(muscleblind-like protein 2 isoform X1) c4( ) c5(314007) c6( ) c7(aj, lU, oj, dR); #c1(MBOAT1) c2(NP_001073949) c3(lysophospholipid acyltransferase 1) c4(36693) c5(36694, 36695, 36696, 36697, 36698, 36699) c6( ) c7(fq); #c1(MBOAT1) c2(XP_006715062) c3(lysophospholipid acyltransferase 1 isoform X1) c4(288567) c5(288568, 288569, 288570, 288571, 288572, 288573) c6( ) c7(fq); #c1(MBOAT1) c2(XP_011512615) c3(lysophospholipid acyltransferase 1 isoform X3) c4(303578) c5(303579, 303580, 303581, 303582, 303583, 303584) c6( ) c7(fq); #c1(MBOAT2) c2(NP_620154) c3(lysophospholipid acyltransferase 2) c4(237374) c5(237375, 237376, 237377, 237378, 237379, 237380) c6( ) c7(q, r); #c1(MBOAT4) c2(XP_011542911) c3(ghrelin O-acyltransferase isoform X1) c4(341513) c5(341514, 341515, 341516, 341517, 341518, 341519) c6( ) c7(bL, aW, iX, az, aT, dq, v, K); #c1(MBP) c2(NP_001020252) c3(myelin basic protein isoform 1) c4(28366) c5(28367, 28368, 28369, 28370, 28371, 28372) c6( ) c7(bM, cZ, hl, bmQ, JZ, aiR, qz, iW, oe, aw, jN, l, ccx, aA, GC, jE, mF, il, V, Fc, eA, ad, hX, ccw, NF, Mo, aBy, uT, ds, ccy, aT, h, abW, ey, dd, bq, H, bU, dF, af, pi, mH, nP, hM, d, dH, aaC, Cw, Ls, lC, eZ, aq, aHU, vs, aF, as, kk, BE, Hv, bN, lY, ck, rR, bkn, ac, iQ, ig, Om, dc, U, uo, Da, eo, k, bee, aiS, hh, y, bt, B, L); #c1(MBP) c2(NP_001020261) c3(myelin basic protein isoform 3) c4( ) c5(28394, 28395, 28396, 28397, 28398) c6( ) c7(bM, cZ, hl, bmQ, JZ, aiR, qz, iW, oe, aw, jN, l, ccx, aA, GC, jE, mF, il, V, Fc, eA, ad, hX, ccw, NF, Mo, aBy, uT, ds, ccy, aT, h, abW, ey, dd, bq, H, bU, dF, af, pi, mH, nP, hM, d, dH, aaC, Cw, Ls, lC, eZ, aq, aHU, vs, af, as, kk, eE, Hv, bN, lY, ck, rR, bkn, ac, iQ, ig, Om, dc, U, uo, Da, eo, k, bee, aiS, hh, y, bt, B, L); #c1(MBP) c2(NP_001020263) c3(myelin basic protein isoform 4) c4( ) c5(28481, 28482, 28483, 28484, 28485, 28486, 28487, 28488, 28489, 28490) c6( ) c7(bM, cZ, hl, bmQ, JZ, aiR, qz, iW, oe, aw, jN, l, ccx, aA, GC, jE, mF, il, V, Fc, eA, ad, hX, ccw, NF, Mo, aBy, uT, ds, ccy, aT, h, abW, ey, dd, bq, H, bU, dF, af, pi, mH, nP, hM, d, dH, aaC, Cw, Ls, lC, eZ, aq, aHU, vs, aF, as, kk, eE, Hv, bN, lY, ck, rR, bkn, ac, iQ, ig, Om, dc, U, uo, Da, eo, k, bee, aiS, hh, y, bt, B, L); #c1(MBP) c2(NP_001020271) c3(Golli-MBP isoform 2) c4(28491) c5(28492, 28493, 28494, 28495, 28496, 28497, 28498, 28499, 28500, 28501) c6( ) c7(bM, cZ, hl, bmQ, JZ, aiR, qz, iW, oe, aw, jN, l, ccx, aA, GC, jE, mF, il, V, Fc, eA, ad, hX, ccw, NF, Mo, aBy, uT, ds, ccy, aT, h, abW, ey, dd, bq, H, bU, dF, af, pi, mH, nP, hM, d, dH, aaC, Cw, Ls, lC, eZ, aq, aHU, vs, aF, as, kk, eE, Hv, bN, lY, ck, rR, bkn, ac, iQ, ig, Om, dc, U, uo, Da, eo, k, bee, aiS, hh, y, bt, B, L); #c1(MBP) c2(NP_001020272) c3(Golli-MBP isoform 1) c4(28502, 28503) c5(28504, 28505, 28506, 28507, 28508, 28509, 28510) c6( ) c7(bM, cZ, hl, bmQ, JZ, aiR, qz, iW, oe, aw, jN, l, ccx, aA, GC, jE, mF, il, V, Fc, eA, ad, hX, ccw, NF, Mo, aBy, uT, ds, ccy, aT, h, abW, ey, dd, bq, H, bU, dF, af, pi, mH, nP, hM, d, dH, aaC, Cw, Ls, lC, eZ, aq, aHU, vs, aF, as, kk, eE, Hv, bN, lY, ck, rR, bkn, ac, iQ, ig, Om, dc, U, uo, Da, eo, k, bee, aiS, hh, y, bt, B, L); #c1(MBP) c2(NP_002376) c3(myelin basic protein isoform 2) c4(127949) c5(127950, 127951, 127952, 127953, 127954, 127955) c6( ) c7(bM, cZ, hl, bmQ, JZ, aiR, qz, iW, oe, aw, jN, l, ccx, aA, GC, jE, mF, il, V, Fc, eA, ad, hX, ccw, NF, Mo, aBy, uT, ds, ccy, aT, h, abW, ey, dd, bq, H, bU, dF, af, pi, mH, nP, hM, d, dH, aaC, Cw, Ls, lC, eZ, aq, aHU, vs, aF, as, kk, eE, Hv, bN, lY, ck, rR, bkn, ac, iQ, ig, Om, dc, U, uo, Da, eo, k, bee, aiS, hh, y, bt, B, L); #c1(MBP) c2(XP_011524312) c3(Golli-MBP isoform X1) c4(318805) c5( ) c6( ) c7(bM, cZ, hl, bmQ, JZ, aiR, qz, iW, oe, aw, jN, l, ccx, aA, GC, jE, mF, il, V, Fc, eA, ad, hX, ccw, NF, Mo, aBy, uT, ds, ccy, aT, h, abW, ey, dd, bq, H, bU, dF, af, pi, mH, nP, hM, d, dH, aaC, Cw, Ls, lC, eZ, aq, aHU, vs, aF, as, kk, eE, Hv, bN, lY, ck, rR, bkn, ac, iQ, ig, Om, dc, U, uo, Da, eo, k, bee, aiS, hh, y, bt, B, L); #c1(MBP) c2(XP_011524313) c3(Golli-MBP isoform X2) c4(318806) c5(318807, 318808) c6( ) c7(bM, cZ, hl, bmQ, JZ, aiR, qz, iW, oe, aw, jN, l, ccx, aA, GC, jE, mF, il, V, Fc, eA, ad, hX, ccw, NF, Mo, aBy, uT, ds, ccy, aT, h, abW, ey, dd, bq, H, bU, dF, af, pi, mH, nP, hM, d, dH, aaC, Cw, Ls, lC, eZ, aq, aHU, vs, aF, as, kk, BE, Hv, bN, lY, ck, rR, bkn, ac, iQ, ig, Om, dc, U, uo, Da, eo, k, bee, aiS, hh, y, bt, B, L); #c1(MBTD1) c2(XP_005257525) c3(MBT domain-containing protein 1 isoform X7) c4(275199) c5(275200, 275201, 275202, 275203, 275204, 275205, 275206, 275207, 275208, 275209, 275210, 275211, 275212) c6( ) c7(dB, Th); #c1(MBTD1) c2(XP_011523237) c3(MBT domain-containing protein 1 isoform X11) c4(317757) c5(317758) c6( ) c7(dB, Th); #c1(MBTD1) c2(XP_011523238) c3(MBT domain-containing protein 1 isoform X12) c4( ) c5(317759, 317760, 317761, 317762, 317763) c6( ) c7(dB, Th); #c1(MBTPS1) c2(NP_003782) c3(membrane-bound transcription factor site-1 protease preproprotein) c4(140769, 140770) c5(140771, 140772, 140773, 140774, 140775, 140776, 140777, 140778, 140779, 140780, 140781, 140782) c6( ) c7(ccz, T, dD, ea, fG, eq, V, ad, iu, hd, ds, aT, ji, K, dF, ae, blm, d, aW, Y, oV, fc, Az, fe, aq, lo, nB, au, cj, Da, dy, eo, cP, B); #c1(MBTPS2) c2(NP_056968) c3(membrane-bound transcription factor site-2 protease) c4(188234, 188235) c5(188236, 188237, 188238, 188239, 188240, 188241, 188242, 188243, 188244, 188245, 188246, 188247) c6( ) c7(ccD, ccG, aOu, MS, acG, ccC, ccA, ccH, bgm, ccE, dr, by, K, lQ, ccF, aTi, B, ccB, bXD, alk); #c1(MC1R) c2(NP_002377) c3(melanocyte-stimulating hormone receptor) c4(127956) c5(127957, 127958, 127959, 127960, 127961, 127962) c6( ) c7(bol, sc, atT, Ve, T, yk, iW, eX, v, qj, Qo, V, cd, ad, aJO, hd, ccJ, ib, Db, afQ, Nn, aT, eV, lQ, an, bsn, ahU, ae, aW, Y, cck, ff, cr, gu, en, bHy, ahV, ccM, hq, qJ, iP, sS, atU, Dy, nB, hk, ccl, agl, ccl, adg, alo, aPG, pC); #c1(MC2R) c2(NP_001278840) c3(adrenocorticotropic hormone receptor) c4(110702) c5(110703, 110704, 110705, 110706, 110707, 110708, 110709, 110710, 110711, 110712) c6( ) c7(bL, T, Ve, aaT, aG, vf, cz, Ja, ci, oH, ad, qC, axy, aVM, aT, iv, bK, K, qM, hK, cC, bgp, ak, li, yo, avH, aWm, s, iw, Hu, es, OW, iA, OD, MN); #c1(MC3R) c2(NP_063941) c3(melanocortin receptor 3) c4(202352) c5(202353, 202354, 202355, 202356, 202357, 202358) c6( ) c7(bL, bVk, Ve, ccN, e, ha, qQ, cz, aj, v, J, aPW, GL, iX, aT, jy, qM, aW, aCK, akq, ig, mf, ei); #c1(MC4R) c2(NP_005903) c3(melanocortin receptor 4) c4(160249) c5(160250, 160251, 160252, 160253, 160254, 160255) c6( ) c7(bL, bM, T, Ve, dD, ea, De, My, qQ, jh, cz, wo, aj, v, afE, eq, q, J, aPW, GL, iX, aoh, aT, jy, K, jo, EL, uV, bSo, BYL, qM, cC, kl, aW, Y, fc, r, dq, cl, fq, bz, bJ, EJ, ck, hq, pO, W, ZT, gX, hL, mf, ei, X); #c1(MC5R) c2(NP_005904) c3(melanocortin receptor 5) c4(160256) c5(160257, 160258, 160259, 160260, 160261, 160262) c6( ) c7(bL, Ve, ccD, v, bK, qM, yA, qW); #c1(MCAM) c2(NP_006491) c3(cell surface glycoprotein MUC18 precursor) c4(165679, 165680, 165681) c5(165682, 165683, 165684, 165685, 165686, 165687, 165688, 165689, 165690, 165691, 165692, 165693, 165694, 165695, 165696, 165697, 165698, 165699) c6( ) c7(oO, bM, T, dA, eR, ci, V, sD, ad, ccP, ib, rw, Nn, aT, GT, dz, UN, K, lQ, zy, hS, ae, cp, aa, UW, et, LC, aEH, Y, aW, g, cl, t, iP, es, Dy, pc, iA, hL, at, nL, pg, m, fD, y, DB); #c1(MCAT) c2(NP_055322) c3(malonyl-CoA-acyl carrier protein transacylase, mitochondrial isoform b precursor) c4(180476) c5(180477, 180478, 180479, 180480, 180481, 180482) c6( ) c7(bL, T, dD, ea, e, jh, bB, vr, aj, eq, jE, fr, eA, J, ad, aT, bOk, h, K, fb, ae, d, nO, qt, aW, hl, cm, iO, W, nB, bE, U, B); #c1(MCAT) c2(NP_775738) c3(malonyl-CoA-acyl carrier protein transacylase, mitochondrial isoform a precursor) c4(250(26) c5(250127, 250128, 250129, 250130, 250131, 250132) c6( ) c7(bL, T, dD, ea, e, jh, bB, vr, aj, eq, jE, fr, eA, J, ad, aT, bOk, h, K, fb, ae, d, nO, qt, aW, hl, cm, iO, W, nB, bE, U, B); #c1(MCC) c2(NP_001078846) c3(colorectal mutant cancer protein isoform 1) c4(37929) c5(37930, 37931, 37932, 37933, 37934, 37935) c6( ) c7(bM, T, qb, al, aA, fr, ci, nA, q, ad, hX, av, ft, aE, Yc, NS, aT, ni, ae, YT, De, BO, s, aW, oV, ff, LZ, r, dN, aF, en, g, bz, bN, bF, gk, ac, eM, bE, z, B); #c1(MCC) c2(NP_002378) c3(colorectal mutant cancer protein isoform 2) c4(127963) c5(127964, 127965, 127966, 127967, (27968, 127969) c6( ) c7(bM, T, qb, al, aA, fr, ci, nA, q, ad, hX, av, ft, aE, Yc, NS, aT, ni, ae, YT, De, BO, s, aW, oV, ff, LZ, r, dN, aF, en, g, bz, bN, bF, gk, ac, eM, bE, z, B); #c1(MCCC1) c2(NP_064551) c3(methylcrotonayl-CoA carboxylase subunit alpha, mitochondrial isoform 1 precursor) c4(202958, 202959, 202960, 202961) c5(202962, 202963, 202964, 202965, 202966, 202967, 202968) c6( ) c7(an, dV, eg); #c1(MCCC1) c2(XP_006713765) c3(methylcrotonayl-CoA carboxylase subunit alpha, mitochondrial isoform X2) c4( ) c5(287337, 287338, 287339, 287340, 287341, 287342, 287343, 287344, 287345, 287346, 287347) c6( ) c7(an, dV, eg); #c1(MCCC1) c2(XP_011511294) c3(methylcrotonoyl-CoA carboxylase subunit alpha, mitochondrial isoform XI) c4(301803) c5(301804) c6( ) c7(an, dV, eg); #c1(MCCC1) c2(XP_011511295) c3(methylcrotonoyl-CoA carboxylase subunit alpha, mitochondrial isoform X3) c4( ) c5(301805, 301806, 301807, 301808, 301809, 301810, 301811, 301812, 301813, 301814, 301815) c6( ) c7(an, dV, eg); #c1(MCCC2) c2(NP_071415) c3(methylcrotonoyl-CoA carboxylase beta chain, mitochondrial) c4(211551) c5(211552, 211553, 211554, 211555, 211556, 211557) c6( ) c7(ccR, ccQ, ka, ac, ki); #c1(MCCC2) c2(XP_005248624) c3(methylcrotonoyl-CoA carboxylase beta chain, mitochondrial isoform X1) c4(268474) c5(268475, 268476, 268477, 268478, 268479, 268480) c6( ) c7(ccR, ccQ, ka, ac, ki); #c1(MCCD1) c2(NP_001011700) c3(mitochondrial coiled-coil domain protein 1 precursor) c4(24709, 24710, 24711) c5(24712, 24713, 24714, 24715, 24716, 24717, 24718, 24719, 24720, 24721, 24722, 24723, 24724, 24725, 24726, 24727, 24728, 24729, 24730, 24731, 24732, 24733, 24734, 24735, 24736, 24737, 24738, 24739, 24740, 24741, 24742, 24743, 24744, 24745, 24746) c6( ) c7(hH, V, aiJ, cr, eZ); #c1(MCEE) c2(NP_115990) c3(methylmalonyl-CoA epimerase, mitochondrial precursor) c4(225126) c5(225127, 225128, 225129, 225130, 225131, 225132) c6( ) c7(ccS, GE); #c1(MCF2) c2(NP_001093325) c3(proto-oncogene DBL isoform a) c4(40232) c5(40233, 40234, 40235, 40236, 40237, 40238, 40239, 40240, 40241, 40242, 40243, 40244, 40245, 40246) c6( ) c7(ya, vT, aW, oe, EQ, dV, bgk, uJ, Ou); #c1(MCF2) c2(NP_001165348) c3(proto-oncogene DBL isoform d) c4(67611) c5(67612, 67613, 67614, 67615, 67616, 67617) c6( ) c7(ya, vT, aW, oe, EQ, dV, bgk, uJ, Ou); #c1(MCF2) c2(NP_001165349) c3(proto-oncogene DBL isoform e) c4(67618) c5(67619, 67620, 67621, 67622, 67623, 67624) c6( ) c7(ya, vT, aW, oe, EQ, dV, bgk, uJ, Ou); #c1(MCF2) c2(NP_001165350) c3(proto-oncogene DBL isoform f) c4(67625) c5(67626, 67627, 67628, 67629, 67630, 67631) c6( ) c7(ya, vT, aW, oe, EQ, dV, bgk, uJ, Ou); #c1(MCF2) c2(NP_005360) c3(proto-oncogene DBL isoform b) c4(155425, 155426) c5(155427, 155428, 155429, 155430, 155431, 155432) c6( ) c7(ya, vT, aW, oe, EQ, dV, bgK, uJ, Ou); #c1(MCF2) c2(XP_005262470) c3(proto-oncogene DBL isoform X2) c4(277964, 277965, 277966) c5(277967, 277968, 277969, 277970, 277971, 277972, 277973, 277974, 277975) c6( ) c7(ya, vT, aW, oe, EQ, dV, bgk, uJ, Ou); #c1(MCF2) c2(XP_006724820) c3(proto-oncogene DBL isoform X5) c4( ) c5(296439, 296440, 296441, 296442, 296443, 296444) c6( ) c7(ya, vT, aW, oe, EQ, dV, bgK, uJ, Ou); #c1(MCF2) c2(XP_011529641) c3(proto-oncogene DBL isoform X1) c4( ) c5(325293, 325294, 325295, 325296, 325297) c6( ) c7(ya, vT, aW, oe, EQ, dV, bgk, uJ, Ou); #c1(MCF2L) c2(NP_001106203) c3(guanine nucleotide exchange factor DBS isoform a) c4(42390, 42391, 42392) c5(42393, 42394, 42395, 42396, 42397, 42398, 42399, 42400, 42401, 42402, 42403, 42404) c6( ) c7(T, b, m, aE, aW, aNJ, e, aT, aU, h, fr, Wm, i, hX); #c1(MCF2L) c2(NP_079255) c3(guanine nucleotide exchange factor DBS isoform b precursor) c4(217593, 217594) c5(217595, 217596, 217597, 217598, 217599, 217600, 217601, 217602, 217603, 217604, 217605, 217606, 217607, 217608, 217609, 217610, 217611, 217612, 217613, 217614, 217615, 217616, 217617, 217618, 217619, 217620, 217621, 217622, 217623, 217624, 217625, 217626) c6( ) c7(T, b, mO, aE, aW, aNJ, e, aT, aU, h, fr, Wm, i, hX); #c1(MCF2L) c2(XP_005268366) c3(guanine nucleotide exchange factor DBS isoform X4) c4(281750) c5(281751, 281752, 281753, 281754, 281755, 281756) c6( ) c7(T, b, mO, aE, aW, aNJ, e, aT, aU, h, fr, Wm, i, hX); #c1(MCF2L) c2(XP_005268373) c3(guanine nucleotide exchange factor DBS isoform X13) c4(281757) c5(281758, 281759, 281760, 281761, 281762, 281763, 281764, 281765, 281766, 281767, 281768) c6( ) c7(T, b, mO, aE, aW, aNJ, e, aT, aU, h, fr, Wm, i, hX); #c1(MCF2L) c2(XP_011535786) c3(guanine nucleotide exchange factor DBS isoform X2) c4(332993) c5( ) c6( ) c7(T, b, mO, aE, aW, aNJ, e, aT, aU, h, fr, Wm, i, hX); #c1(MCF2L) c2(XP_011535787) c3(guanine nucleotide exchange factor DBS isoform X3) c4(332994) c5( ) c6( ) c7(T, b, mO, aE, aW, aNJ, e, aT, aU, h, fr, Wm, i, hX); #c1(MCF2L) c2(XP_011535791) c3(guanine nucleotide exchange factor DBS isoform X8) c4(332995) {00886765] c5(332996, 332997, 332998, 332999, 333000, 333001, 333002, 333003, 333004, 333005) c6( ) c7(T, b, mO, aE, aW, aNJ, e, aT, aU, h, fr, Wm, i, hX); #c1(MCF2L) c2(XP_011535795) c3(guanine nucleotide exchange factor DBS isoform X14) c4(333006, 333007) c5(333008, 333009, 333010, 333011, 333012) c6( ) c7(T, b, mO, aE, aW, aNJ, e, aT, aU, h, fr, Wm, i, hX); #c1(MCF2L2) c2(NP_055893) c3(probable guanine nucleotide exchange factor MCF2L2) c4(184247, 184248, 184249, 184250, 184251) c5(184252, 184253, 184254, 184255, 184256, 184257, 184258, 184259, 184260, 184261, 184262, 184263, 184264, 184265, 184266, 184267, 184268, 184269, 184270, 184271, 184272, 184273, 184274, 184275, 184276) c6( ) c7(bL, ccT, cZ, kl, A, eR, xZ, dd, hH); #c1(MCF2L2) c2(XP_011510889) c3(probable guanine nucleotide exchange factor MCF2L2 isoform X2) c4( ) c5(301352, 301353, 301354, 301355, 301356) c6( ) c7(bL, ccT, cZ, kl, A, eR, xZ, dd, hH); #c1(MCFD2) c2(NP_001164979) c3(multiple coagulation factor deficiency protein 2 isoform A precursor) c4(67182, 67183) c5(67184, 67185, 67186, 67187, 67188, 67189, 67190, 67191, 67192, 67193, 67194, 67195, 67196, 67197, 67198, 67199, 67200, 67201, 67202, 67203, 67204, 67205, 67206, 67207, 67208, 67209, 67210, 67211, 67212, 67213, 67214, 67215, 67216, 67217, 67218, 67219, 67220, 67221, 67222, 67223, 67224, 67225, 67226, 67227, 67228) c6(67229) c7(NU, aUK, wu, qh, acb, oY, dX, wm, bqA, ccU, aUC, bpU); #c1(MCFD2) c2(NP_001164981) c3(multiple coagulation factor deficiency protein 2 isoform B) c4( ) c5(67230, 67231, 67232, 67233, 67234, 67235, 67236, 67237, 67238, 67239, 67240, 67241, 67242, 67243) c6(67244, 67245, 67246) c7(NU, aUK, wu, qh, acb, oY, dX, wm, bqA, ccU, aUC, bpU); #c1(MCFD2) c2(NP_001164982) c3(multiple coagulation factor deficiency protein 2 isoform C) c4(67247, 67248) c5(67249, 67250, 67251, 67252, 67253, 67254, 67255, 67256, 67257, 67258, 67259) c6( ) c7(NU, aUK, wu, qh, acb, oY, dX, wm, bqA, ccU, aUC, bpU); #c1(MCHR1) c2(NP_005288) c3(melanin-concentrating hormone receptor 1) c4(154704, 154705) c5(154706, 154707, 154708, 154709, 154710, 154711, 154712, 154713, 154714, 154715, 154716, 154717, 154718) c6( ) c7(bl, mS, bS, v, ct, qj, mW, bK, K, mY, mV, mU, s, Y, cr, Z, cm, mZ, mX, cA, uJ, mT, pC); #c1(MCHR2) c2(NP_001035269) c3(melanin-concentrating hormone receptor 2) c4(32733) c5(32734, 32735, 32736, 32737, 32738, 32739, 32740, 32741, 32742, 32743, 32744, 32745, 32746, 32747) c6( ) c7(e, qQ, EQ, mS, v, afE, abb, aE, mW, ql, BJ, bK, qM, mY, he, KU, mV, cY, ml, s, lh, Eg, mZ, mX, Bv, aLA, pJ, mT); #c1(MCIDAS) c2(NP_001177716) c3(multicilin) c4(72894) c5(72895, 72896, 72897, 72898, 72899, 72900) c6( ) c7(ak, sO, yy, aaw, PG, K, af, eu); #c1(MCM10) c2(NP_060988) c3(protein MCM10 homolog isoform 2) c4(198902, 198903) c5(198904, 198905, 198906, 198907, 198908, 198909, 198910, 198911, 198912, 198913, 198914, 198915, 198916) c6( ) c7(bL, T, ac, pN, if, e, aw, j, qN, it, eu); #c1(MCM10) c2(NP_877428) c3(protein MCM10 homolog isoform 1) c4(256802) c5(256803, 256804, 256805, 256806, 256807, 256808, 256809, 256810, 256811, 256812) c6( ) c7(bL, T, ac, pN, if, e, aw, j, qN, it, eu); #c1(MCM2) c2(NP_004517) c3(DNA replication licensing factor MCM2) c4(147136, 147137, 147138) c5(147139, 147140, 147141, 147142, 147143, 147144, 147145, 147146, 147147, 147148, 147149, 147150, 147151, 147152, 147153, 147154, 147155, 147156) c6( ) c7(bL, T, aGn, id, Uc, if, al, jh, fd, bB, n, fr, ci, qN, aql, hF, q, ad, er, ip, XX, aT, h, dL, K, pk, ae, jf, ayT, kl, B, s, hG, aW, Y, aJD, r, g, t, iK, bw, es, gk, bP, eM, bE, bkv, cj, k, m, bt, X); #c1(MCM3) c2(NP_001257401) c3(DNA replication licensing factor MCM3 isoform 2) c4(95344) c5(95345, 95346, 95347, 95348, 95349, 95350, 95351, 95352, 95353, 95354, 95355, 95356, 95357, 95358, 95359, 95360, 95361) c6( ) c7(ck, jf, NK, T, Dy, dB, Y, aT, h, ci, B, ip); #c1(MCM3) c2(NP_002379) c3(DNA replication licensing factor MCM3 isoform 1) c4(127970, 127971, 127972, 127973) c5(127974, 127975, 127976, 127977, 127978, 127979, 127980, 127981, 127982, 127983, (27984, 127985, 127986) c6( ) c7(ck, jf, NK, T, Dy, dB, Y, aT, h, ci, B, ip); #c1(MCM3AP) c2(XP_006724127) c3(germinal-center associated nuclear protein isoform X1) c4(295772, 295773, 295774) c5(295775, 295776, 295777, 295778, 295779, 295780, 295781, 295782, 295783, 295784, 295785, 295786, 295787, 295788, 295789, 295790, 295791, 295792, 295793, 295794, 295795, 295796) c6( ) c7(T, Y, eX, jh, eV, bU, eA); #c1(MCM4) c2(NP_877423) c3(DNA replication licensing factor MCM4) c4(256790, 256791) c5(256792, 256793, 256794, 256795, 256796, 256797, 256798, 256799, 256800, 256801) c6( ) c7(ck, jf, T, Dy, eM, bP, aX, if, oW, aT, cj, KN, fd, qN, g, ccV, ip); #c1(MCM4) c2(XP_005251291) c3(DNA replication licensing factor MCM4 isoform X1) c4( ) c5(270865, 270866, 270867, 270868, 270869, 270870) c6( ) c7(ck, jf, T, Dy, eM, bP, aX, if, oW, aT, cj, KN, fd, qN, g, ccV, ip); #c1(MCM5) c2(NP_006730) c3(DNA replication licensing factor MCM5) c4(167526, 167527) c5(167528, 167529, 167530, 167531, 167532, 167533, 167534, 167535, 167536, 167537, 167538, 167539) c6( ) c7(T, hd, akd, eL, aoA, cn, cj, aT, n, fd, dL, lQ, qN, hu, g, ae, X, ad, ip); #c1(MCM5) c2(XP_006724305) c3(DNA replication licensing factor MCM5 isoform X1) c4(295904, 295905) c5(295906, 295907, 295908, 295909, 295910, 295911, 295912, 295913, 295914, 295915, 295916, 295917, 295918) c6( ) c7(T, hd, akd, eL, aoA, cn, cj, aT, n, fd, dL, lQ, qN, hu, g, ae, X, ad, ip); #c1(MCM6) c2(NP_005906) c3(DNA replication licensing factor MCM6) c4(160263) c5(160264, 160265, 160266, 160267, 160268, 160269) c6( ) c7(es, aMa, aAS, aMb, jh, aT, fr, ci, t, ip); #c1(MCM7) c2(NP_005907) c3(DNA replication licensing factor MCM7 isoform 1) c4(160270, 160271, 160272, 160273, 160274) c5(160275, 160276, 160277, 160278, 160279, 160280, 160281, 160282, 160283, 160284, 160285, 160286, 160287, 160288, 160289, 160290, 160291, 160292, 160293, 160294) c6( ) c7(aJJ, T, id, Uc, if, al, jh, aA, bB, n, fd, fr, ci, qN, hF, iJ, q, ad, er, ip, XX, hd, aE, aT, h, dL, K, pk, ae, eL, jf, ayT, kl, s, hG, aW, ff, aJD, r, aF, en, cl, g, cm, bw, pb, bN, es, gk, Dy, eM, bP, bE, bkv, hr, cj, hL, YK, k, m, bt, B); #c1(MCM7) c2(XP_005250405) c3(DNA replication licensing factor MCM7 isoform X1) c4( ) c5(270291, 270292, 270293, 270294, 270295, 270296, 270297, 270298, 270299, 270300) c6( ) c7(aJJ, T, id, Uc, if, al, jh, aA, bB, n, fd, fr, ci, qN, hF, iJ, q, ad, er, ip, XX, hd, aE, aT, h, dL, K, pk, ae, eL, jf, ayT, kl, s, hG, aW, ff, aJD, r, aF, en, cl, g, cm, bw, pb, bN, es, gk, Dy, eM, bP, bE, bkv, hr, cj, hL, YK, k, m, bt, B); #c1(MCM8) c2(NP_001268449) c3(DNA helicase MCM8 isoform 1) c4(99944) c5(99945, 99946, 99947, 99948, 99949, 99950) c6( ) c7(nJ, y, Y, gv, ev); #c1(MCM8) c2(NP_001268450) c3(DNA helicase MCM8 isoform 3) c4(99951) c5(99952, 99953, 99954, 99955, 99956, 99957) c6( ) c7(nJ, y, Y, gv, ev); #c1(MCM8) c2(NP_001268451) c3(DNA helicase MCM8 isoform 4) c4(99958, 99959) c5(99960, 99961, 99962, 99963, 99964, 99965) c6( ) c7(nJ, y, Y, gv, ev); #c1(MCM8) c2(NP_877954) c3(DNA helicase MCM8 isoform 2) c4(257035) c5(257036, 257037, 257038, 257039, 257040, 257041) c6( ) c7(nJ, y, Y, gv, ev); #c1(MCM8) c2(XP_011527689) c3(DNA helicase MCM8 isoform X1) c4( ) c5(322611, 322612, 322613, 322614, 322615, 322616) c6( ) c7(nJ, y, Y, gv, ev); #c1(MCM9) c2(NP_060166) c3(DNA helicase MCM9 isoform 1) c4(194583, 194584, 194585, 194586) c5(194587, 194588, 194589, 194590, 194591, 194592, 194593, 194594, 194595, 194596, 194597, 194598) c6( ) c7(B); #c1(MCM9) c2(NP_694987) c3(DNA helicase MCM9 isoform 2) c4(245588) c5(245589, 245590, 245591, 245592, 245593, 245594, 245595, 245596, 245597, 245598, 245599, 245600, 245601, 245602, 245603, 245604, 245605, 245606) c6( ) c7(B); #c1(MCMBP) c2(NP_001243307) c3(mini-chromosome maintenance complex-binding protein isoform 2) c4(90402) c5(90403, 90404, 90405, 90406, 90407, 90408) c6( ) c7(oe); #c1(MCMBP) c2(NP_079110) c3(mini-chromosome maintenance complex-binding protein isoform 1) c4(217070) c5(217071, 217072, 217073, 217074, 217075, 217076) c6( ) c7(De); #c1(MCOLN1) c2(NP_065394) c3(mucolipin-1) c4(204713, 204714) c5(204715, 204716, 204717, 204718, 204719, 204720, 204721, 204722, 204723, 204724, 204725, 204726) c6( ) c7(asp, Am, xs, cV, U, h, sH, Lq, beZ, aR); #c1(MCOLN3) c2(XP_011540041) c3(mucolipin-3 isoform X2) c4(338340, 338341) c5(338342, 338343) c6( ) c7(aQi); #c1(MCOLN3) c2(XP_011540042) c3(mucolipin-3 isoform X3) c4(338344) c5(338345, 338346, 338347, 338348, 338349, 338350, 338351, 338352, 338353, 338354) c6( ) c7(aQi); #c1(MCOLN3) c2(XP_011540043) c3(mucolipin-3 isoform X4) c4( ) c5(338355, 338356, 338357, 338358, 338359, 338360) c6( ) c7(aQi); #c1(MCPH1) c2(NP_00166045) c3(microcephalin isoform 2) c4(68129) c5( ) c6( ) c7(gV, T, dD, aC, ea, GC, j, oH, alQ, ci, nA, eq, AD, zo, eA, aE, ccW, aT, rz, fb, aij, aii, d, hG, qt, aW, WE, nO, g, QK, t, cm, bN, m); #c1(MCPH1) c2(NP_001166046) c3(microcephalin isoform 3) c4(68130) c5(68131, 68132, 68133, 68134, 68135, 68136) c6( ) c7(gV, T, dD, aC, ea, GC, j, oH, alQ, ci, nA, eq, AO, zo, eA, aE, ccW, aT, rz, fb, aij, aii, d, hG, qt, aW, WE, nO, g, QK, t, cm, bN, m); #c1(MCPH1) c2(NP_078872) c3(microcephalin isoform 1) c4(215854) c5(215855, 215856, 215857, 215858, 215859, 215860) c6( ) c7(gV, T, dD, aC, ea, GC, j, oH, alQ, ci, nA, eq, AO, zo, eA, aE, ccW, aT, rz, fb, aij, aii, d, hG, qt, aW, WE, nO, g, QK, t, cm, bN, m); #c1(MCPH1) c2(XP_011533061) c3(microcephalin isoform X5) c4( ) c5(329269, 329270, 329271, 329272, 329273, 329274) c6( ) c7(gV, T, dD, aC, ea, GC, j, oH, alQ, ci, nA, eq, AO, zo, eA, aE, ccW, aT, rz, fb, aij, aii, d, hG, qt, aW, WE, nO, g, QK, t, cm, bN, m); #c1(MCRS1) c2(NP_001012300) c3(microspherule protein 1 isoform 2) c4(24846, 24847) c5(24848, 24849, 24850, 24851, 24852, 24853) c6( ) c7(T, d, OW, eM, r, K, eA, f, bz, q); #c1(MCRS1) c2(NP_001265270) c3(microspherule protein 1 isoform 3) c4( ) c5(98089, 98090, 98091, 98092, 98093) c6( ) c7(T, d, OW, eM, r, K, eA, f, bz, q); #c1(MCRS1) c2(XP_005268629) c3(microspherule protein 1 isoform X1) c4(281970, 281971, 281972, 281973) c5(281974, 281975, 281976, 281977, 281978, 281979, 281980, 281981, 281982, 281983, 281984, 281985, 281986, 281987, 281988, 281989, 281990, 281991, 281992, 281993, 281994, 281995, 281996, 281997, 281998, 281999, 282000, 282001, 282002) c6( ) c7(T, d, OW, eM, r, K, eA, f, bz, q); #c1(MCTP1) c2(NP_001002796) c3(multiple C2 and transmembrane domain-containing protein 1 isoform S) c4(19601, 19602) c5(19603, 19604, 19605, 19606, 19607, 19608, 19609, 19610, 19611, 19612, 19613, 19614) c6( ) c7(aw, fq, bK); #c1(MCTP1) c2(NP_001284706) c3(multiple C2 and transmembrane domain-containing protein 1 isoform 3) c4(112358, 112359) c5(112360, 112361, 112362, 112363, 112364, 112365, 112366, 112367, 112368, 112369, 112370, 112371, 112372, 112373, 112374, 112375, 112376, 112377) c6( ) c7(aw, fq, bK); #c1(MCTP1) c2(NP_078993) c3(multiple CZ and transmembrane domain-containing protein 1 isoform L) c4(216459, 216460) c5(216461, 216462, 216463, 216464, 216465, 216466, 216467, 216468, 216469, 216470, 216471, 216472) c6( ) c7(aw, fq, bK); #c1(MCTP1) c2(XP_005272147) c3(multiple CZ and transmembrane domain-containing protein 1 isoform XII) c4(283730) c5( ) c6( ) c7(aw, fq, bK); #c1(MCTP2) c2(NP_001153115) c3(multiple C2 and transmembrane domain-containing protein 2 isoform 2) c4(60436) c5(60437, 60438, 60439, 60440, 60441, 60442) c6( ) c7(Kc, eR, Gp, aw, bO, v, j, Z, aY, fq, hH); #c1(MCTP2) c2(NP_001153116) c3(multiple C2 and transmembrane domain-containing protein 2 isoform 3) c4( ) c5(60443, 60444, 60445, 60446, 60447, 60448, 60449, 60450, 60451) c6( ) c7(Kc, eR, Gp, aw, bO, v, j, Z, aY, fq, hH); #c1(MCTP2) c2(NP_060819) c3(multiple C2 and transmembrane domain-containing protein 2 isoform 1) c4(197979) c5(197980, 197981, 197982, 197983, 197984, 197985) c6( ) c7(Kc, eR, Gp, aw, bO, v, j, Z, aY, fq, hH); #c1(MCTP2) c2(XP_011520072) c3(multiple CZ and transmembrane domain-containing protein 2 isoform X2) c4(314970) c5( ) c6( ) c7(Kc, eR, Gp, aw, bO, v, j, Z, aY, fq, hH); #c1(MCTP2) c2(XP_011520075) c3(multiple CZ and transmembrane domain-containing protein 2 isoform X3) c4(314971) c5(314972) c6( ) c7(Kc, eR, Gp, aw, bO, v, j, Z, aY, fq, hH); #c1(MCTP2) c2(XP_011520076) c3(multiple C2 and transmembrane domain-containing protein 2 isoform X4) c4( ) c5(314973, 314974, 314975, 314976, 314977, 314978) c6( ) c7(Kc, eR, Gp, aw, bO, v, j, Z, aY, fq, hH); #c1(MCTS1) c2(NP_001131026) c3(malignant T-cell-amplified sequence 1 isoform 2) c4(52538) c5(52539, 52540, 52541, 52542, 52543, 52544) c6( ) c7(T, fM, jh, fd, ci, eA, ad, mj, aT, DO, jy, K, hg, fb, ae, d, aW, eH, fe, eF, cm, bz, bo); #c1(MCTS1) c2(NP_054779) c3(malignant T-cell-amplified sequence 1 isoform 1) c4(177776) c5(177777, 177778, 177779, 177780, 177781, 177782) c6( ) c7(T, fM, jh, fd, ci, eA, ad, mj, aT, DO, jy, K, hg, fb, ae, d, aW, eH, fe, eF, cm, bz, bo); #c1(MCU) c2(NP_001257608) c3(calcium uniporter protein, mitochondrial isoform 2 precursor) c4(95510) c5(95511, 95512, 95513, 95514, 95515, 95516) c6( ) c7(T, ccX); #c1(MCU) c2(NP_001257609) c3(calcium uniporter protein, mitochondrial isoform 3) c4(95517) c5(95518, 95519, 95520, 95521, 95522, 95523) c6( ) c7(T, ccX); #c1(MCU) c2(NP_612366) c3(calcium uniporter protein, mitochondrial isoform 1 precursor) c4(236277) c5(236278, 236279, 236280, 236281, 236282, 236283) c6( ) c7(T, ccX); #c1(MCUR1) c2(NP_001026883) c3(mitochondrial calcium uniporter regulator 1) c4(29828) c5(29829, 29830, 29831, 29832, 29833, 29834) c6( ) c7(ey); #c1(MCUR1) c2(XP_011513104) c3(mitochondrial calcium uniporter regulator 1 isoform X1) c4(304826) c5(304827, 304828, 304829, 304830, 304831, 304832) c6( ) c7(ey); #c1(MDC1) c2(NP_055456) c3(mediator of DNA damage checkpoint protein 1) c4(181279, 181280) c5(181281, 181282, 181283, 181284, 181285, 181286, 181287, 181288, 181289, 181290, 181291, 181292, 181293, 181294, 181295, 181296, 181297, 181298, 181299, 181300, 181301, 181302, 181303, 181304) c6( ) c7(T, aC, eZ, cj, g, hH, ad); #c1(MDC1) c2(XP_005249549) c3(mediator of DNA damage checkpoint protein 1 isoform X4) c4( ) c5(269570, 269571, 269572, 269573, 269574, 269575, 269576, 269577, 269578, 269579, 269580, 269581, 269582, 269583, 269584, 269585, 269586, 269587, 269588, 269589, 269590, 269591, 269592, 269593, 269594, 269595, 269596, 269597, 269598, 269599, 269600, 269601, 269602, 269603, 269604, 269605, 269606, 269607, 269608, 269609, 269610, 269611, 269612, 269613, 269614, 269615, 269616, 269617, 269618, 269619, 269620, 269621, 269622, 269623, 269624, 269625, 269626, 269627, 269628, 269629, 269630, 269631, 269632, 269633, 269634, 269635, 269636, 269637, 269638, 269639, 269640, 269641, 269642, 269643, 269644, 269645, 269646, 269647, 269648, 269649, 269650, 269651, 269652, 269653, 269654, 269655, 269656) c6( ) c7(T, aC, eZ, cj, g, hH, ad); #c1(MDC1) c2(XP_005249555) c3(mediator of DNA damage checkpoint protein 1 isoform X9) c4(269657) c5(269658, 269659, 269660, 269661, 269662, 269663, 269664, 269665, 269666, 269667, 269668, 269669, 269670, 269671, 269672, 269673, 269674, 269675, 269676, 269677, 269678, 269679, 269680, 269681, 269682, 269683, 269684, 269685, 269686, 269687, 269688, 269689, 269690, 269691, 269692, 269693, 269694, 269695, 269696, 269697, 269698, 269699, 269700, 269701, 269702, 269703, 269704) c6( ) c7(T, aC, eZ, cj, g, hH, ad); #c1(MDC1) c2(XP_005275549) c3(mediator of DNA damage checkpoint protein 1 isoform X4) c4( ) c5(285613, 285614) c6( ) c7(T, aC, eZ, cj, g, hH, ad); #c1(MDCI) c2(XP_005275555) c3(mediator of DNA damage checkpoint protein 1 isoform X9) c4(285615) c5(285616, 285617, 285618, 285619, 285620, 285621, 285622, 285623, 285624, 285625, 285626, 285627) c6( ) c7(T, aC, eZ, cj, g, hH, ad); #c1(MDCI) c2(XP_005275686) c3(mediator of DNA damage checkpoint protein 1 isoform X4) c4(285689) c5(285690, 285691, 285692, 285693, 285694, 285695) c6( ) c7(T, aC, eZ, cj, g, hH, ad); #c1(MDCI) c2(XP_005275692) c3(mediator of DNA damage checkpoint protein 1 isoform X9) c4(285696) c5(285697, 285698, 285699, 285700, 285701, 285702, 285703) c6( ) c7(T, aC, eZ, cj, g, hH, ad); #c1(MDCI) c2(XP_011546255) c3(mediator of DNA damage checkpoint protein 1 isoform X3) c4(344937, 344938, 344939, 344940, 344941, 344942, 344943, 344944, 344945, 344946, 344947) c5(344948, 344949, 344950, 344951, 344952, 344953, 344954, 344955, 344956, 344957, 344958, 344959, 344960, 344961, 344962, 344963, 344964, 344965, 344966, 344967, 344968, 344969, 344970, 344971, 344972, 344973, 344974, 344975, 344976, 344977, 344978, 344979, 344980, 344981, 344982, 344983, 344984, 344985, 344986, 344987, 344988, 344989, 344990, 344991, 344992, 344993, 344994, 344995, 344996, 344997, 344998, 344999, 345000, 345001, 345002, 345003, 345004, 345005, 345006, 345007, 345008, 345009, 345010, 345011, 345012, 345013, 345014, 345015, 345016, 345017, 345018, 345019, 345020, 345021, 345022, 345023, 345024, 345025, 345026, 345027, 345028, 345029, 345030, 345031, 345032, 345033, 345034, 345035, 345036, 345037, 345038, 345039, 345040, 345041, 345042, 345043, 345044, 345045, 345046, 345047, 345048, 345049, 345050, 345051, 345052) c6( ) c7(T, aC, eZ, cj, g, hH, ad); #c1(MDC1) c2(XP_011546622) c3(mediator of DNA damage checkpoint protein 1 isoform X6) c4(345442, 345443) c5(345444, 345445, 345446, 345447, 345448, 345449, 345450, 345451, 345452, 345453) c6( ) c7(T, aC, eZ, cj, g, hH, ad); #c1(MDCI) c2(XP_011546808) c3(mediator of DNA damage checkpoint protein 1 isoform X6) c4(345700) c5(345701, 345702, 345703, 345704, 345705, 345706, 345707, 345708, 345709, 345710, 345711, 345712, 345713, 345714, 345715, 345716, 345717, 345718, 345719, 345720, 345721, 345722, 345723, 345724, 345725, 345726, 345727, 345728, 345729, 345730) c6( ) c7(T, aC, eZ, cj, g, hH, ad); #c1(MDF1) c2(NP_001287733) c3(myoD family inhibitor isoform 1) c4(112703, 112704, 112705, 112706, 112707) c5(112708, 112709, 112710, 112711, 112712, 112713, 112714, 112715, 112716, 112717, 112718, 112719, 112720, 112721, 112722, 112723, 112724, 112725, 112726, 112727, 112728, 112729, 112730, 112731, 112732, 112733, 112734, 112735, 112736, 112737, 112738, 112739, 112740, (12741, 112742, 112743, 112744, 112745) c6( ) c7(e); #c1(MDF1C) c2(NP_001159817) c3(myoD family inhibitor domain-containing protein isoform p32) c4(64792) c5(64793, 64794, 64795, 64796, 64797) c6( ) c7(abX); #c1(MDF1C) c2(NP_001159818) c3(myoD family inhibitor domain-containing protein isoform c) c4( ) c5(64798, 64799, 64800) c6( ) c7(abX); #c1(MDF1C) c2(NP_951038) c3(myoD family inhibitor domain-containing protein isoform p40) c4(261008, 261009) c5(261010, 261011, 261012, 261013, 261014, 261015) c6( ) c7(abX); #c1(MDGA1) c2(NP_705691) c3(MAM domain-containing glycosylphosphatidylinositol anchor protein 1 precursor) c4(246189, 246190) c5(246191, 246192, 246193, 246194, 246195, 246196, 246197, 246198, 246199, 246200, 246201, 246202) c6( ) c7(bk, bO); #c1(MDGA1) c2(XP_006715119) c3(MAM domain-containing glycosylphosphatidylinositol anchor protein 1 isoform X1) c4(288745) c5(288746, 288747, 288748, 288749, 288750, 288751) c6( ) c7(bK, bO); #c1(MDGA2) c2(NP_001106970) c3(MAM domain-containing glycosylphosphatidylinositol anchor protein 2 isoform 1 precursor) c4(42724, 42725) c5(42726, 42727, 42728, 42729, 42730, 42731, 42732, 42733, 42734, 42735, 42736, 42737) c6( ) c7(oe, eZ, akf, agJ, y, bN, Ef); #c1(MDGA2) c2(NP_878250) c3(MAM domain-containing glycosylphosphatidylinositol anchor protein 2 isoform 2) c4(257063) c5( ) c6( ) c7(oe, eZ, akf, agJ, y, bN, Ef); #c1(MDGA2) c2(XP_011534821) c3(MAM domain-containing glycosylphosphatidylinositol anchor protein 2 isoform X1) c4(331312) c5(331313, 331314, 331315, 331316, 331317, 331318, 331319, 331320, 331321, 331322) c6( ) c7(oe, eZ, akf, agJ, y, bN, Ef); #c1(MDGA2) c2(XP_011534825) c3(MAM domain-containing glycosylphosphatidylinositol anchor protein 2 isoform X5) c4( ) c5(331323) c6( ) c7(oe, eZ, akf, agJ, y, bN, Ef); #c1(MDGA2) c2(XP_011534827) c3(MAM domain-containing glycosylphosphatidylinositol anchor protein 2 isoform X7) c4( ) c5(331324, 331325, 331326, 331327, 331328, 331329) c6( ) c7(oe, eZ, akf, agJ, y, bN, Ef); #c1(MDH1) c2(NP_001186040) c3(malate dehydrogenase, cytoplasmic isoform 1) c4(76923) c5(76924, 76925, 76926, 76927, 76928, 76929) c6( ) c7(d, c, cz); #c1(MDH1) c2(NP_001186041) c3(malate dehydrogenase, cytoplasmic isoform 3) c4( ) c5(76930, 76931, 76932, 76933, 76934) c6( ) c7(d, c, cz); #c1(MDH1) c2(NP_005908) c3(malate dehydrogenase, cytoplasmic isoform 2) c4(160295, 160296) c5(160297, 160298, 160299, 160300, 160301, 160302, 160303, 160304) c6( ) c7(d, c, cz); #c1(MDH2) c2(NP_005909) c3(malate dehydrogenase, mitochondrial isoform 1 precursor) c4(160305) c5(160306, 160307, 160308, 160309, 160310, 160311) c6( ) c7(T, zQ, iE, xZ, aD, eu, aw, h, ci, zP, ae, cd, ad, FT); #c1(MDK) c2(NP_001257481) c3(midkine isoform b precursor) c4(95489) c5(95490, 95491, 95492, 95493, 95494, 95495, 95496, 95497) c6( ) c7(eW, cZ, T, if, aw, ht, aA, ajp, n, bB, ci, qN, q, ad, aDl, ai, dP, er, eA, b, ip, ot, aE, Db, SU, mj, iu, aeO, aT, h, cy, x, YB, dL, K, hP, dd, ae, af, eu, pl, eL, eN, jf, yo, d, aeR, s, vT, aW, ff, eH, r, kp, aF, aiz, en, as, eF, t, cm, bz, aZz, ek, hq, sS, wg, nM, eM, Ew, dB, bE, eD, X, hL, cj, Zn, m, k, i, y, B, ao, hS); #c1(MDK) c2(XP_011518418) c3(midkine isoform X1) c4(312396, 312397, 312398) c5(312399, 312400, 312401, 312402, 312403, 312404, 312405, 312406, 312407, 312408, 312409, 312410, 312411, 312412, 312413, 312414, 312415, 312416, 312417, 312418, 312419, 312420, 312421, 312422, 312423, 312424, 312425, 312426, 312427, 312428, 312429, 312430, 312431, 312432, 312433, 312434, 312435, 312436) c6( ) c7(eW, cZ, T, if, aw, ht, aA, ajp, n, bB, ci, qN, q, ad, aDl, ai, dP, er, eA, b, ip, ot, aE, Db, SU, mj, iu, aeO, aT, h, cy, x, YB, dL, K, hP, dd, ae, af, eu, pl, eL, eN, jf, yo, d, aeR, s, vT, aW, ff, eH, r, kp, aF, aiz, en, as, eF, t, cm, bz, aZz, ek, hq, sS, wg, nM, eM, Ew, dB, bE, eD, X, hl, cj, Zn, m, k, i, y, B, ao, hS); #c1(MDM1) c2(NP_001191957) c3(nuclear protein MDM1 isoform 4) c4(81894) c5(81895, 81896, 81897, 81898, 81899, 81900) c6( ) c7(aj, nw, bk, W, j); #c1(MDM1) c2(NP_001191958) c3(nuclear protein MDM1 isoform 3) c4(81901) c5(81902, 81903, 81904, 81905, 81906) c6( ) c7(aj, nw, bk, W, j); #c1(MDM1) c2(NP_059136) c3(nuclear protein MDM1 isoform 1) c4(193223) c5(193224, 193225, 193226, 193227, 193228, 193229) c6( ) c7(aj, nw, bK, W, j); #c1(MDM1) c2(NP_064513) c3(nuclear protein MDM1 isoform 2) c4(202816) c5(202817, 202818, 202819, 202820, 202821, 202822, 202823) c6( ) c7(aj, nw, bk, W, j); #c1(MDM2) c2(NP_001138809) c3(E3 ubiquitin-protein ligase Mdm2 isoform g) c4(57775, 57776) c5(57777, 57778, 57779, 57780, 57781, 57782, 57783, 57784, 57785, 57786, 57787, 57788) c6( ) c7(T, aC, aiH, jW, fR, pf, hV, ea, if, al, cdf, bB, fd, qj, ci, aJa, ip, x, bv, bHS, gJ, aE, Bc, aeO, Qk, ajv, ja, cda, bnt, K, hu, pk, eL, cp, eG, zQ, aW, aEl, Ls, az, hl, QB, CT, kt, Qm, g, as, eF, ve, cm, bw, ccZ, iP, y, fT, eM, nB, AM, iA, iT, vA, cj, PV, k, m, cE, iu, alM, dD, gU, Qj, wu, sw, iB, ht, qy, n, fr, ct, Qh, qN, cde, co, Bl, jz, apS, Bj, aeX, bUP, h, eV, dL, Gw, Ha, dx, pl, cY, awV, FZ, TH, hQ, GA, aq, kp, yq, pm, mo, iN, Zj, op, oU, afb, ar, KT, iO, YE, bP, bbz, dB, hk, eD, Pl, Qu, Vy, hL, hj, at, MW, azr, Qz, WL, Pr, B, it, fM, dA, yk, ex, aA, hy, C, bX, Qo, il, lu, q, eA, eJ, ccY, amg, lg, hW, nv, qK, yg, aT, aHs, ae, ne, jf, Qr, vT, NE, nO, Y, Lf, iU, r, lo, en, bu, cl, t, iK, vk, ck, sS, cdd, kP, Dy, nM, iM, QG, au, im, zu, cdb, bt, rA, hS, On, gx, gV, kh, Gl, id, Mb, atY, iW, jh, KO, bS, Gn, jE, eq, Fc, iJ, ad, kL, hX, qC, av, Tg, hd, F, ib, cdc, bjn, Db, iC, lC, hP, bwV, wm, fb, nJ, WW, d, BN, BO, hG, qt, je, ff, eH, yy, aeV, Ej, aF, iV, Z, iR, bz, es, gk, eL, RV, pc, zr, Ew, bE, amS, sG, Hs, QC, bC, Gu, aeH, X, rk, BA, pC); #c1(MDM2) c2(NP_001138811) c3(E3 ubiquitin-protein ligase Mdm2 isoform h) c4(57853, 57854) c5(57855, 57856, 57857, 57858, 57859, 57860, 57861, 57862, 57863, 57864, 57865, 57866) c6( ) c7(T, aC, aiH, jW, fR, pf, hV, ea, if, al, cdf, bB, fd, qj, ci, aJa, ip, x, bv, bHS, gJ, aE, Bc, aeO, Qk, ajv, ja, cda, bnt, K, hu, pk, eL, cp, eG, zQ, aW, aEl, Ls, az, hl, QB, CT, kt, Um, g, as, eF, ve, cm, bw, ccZ, iP, y, fT, eM, nB, AM, iA, iT, vA, cj, PV, k, m, cE, iu, alM, dD, gU, Qj, wu, sw, iB, ht, qy, n, fr, ct, Qh, qN, cde, co, Bl, jz, apS, Bj, aeX, bUP, h, eV, dL, Gw, Ha, dx, pl, cY, awV, FZ, TH, hQ, GA, aq, kp, yq, pm, mo, iN, Zj, op, oU, afb, ar, kT, iO, YE, bP, bbz, dB, hk, eD, Pl, Qu, Vy, hL, hj, at, MW, azr, Qz, WL, Pr, B, it, fM, dA, yk, eX, aA, hy, C, bX, Qo, il, lu, q, eA, eJ, ccY, amg, lg, hW, nv, qK, yg, aT, aHs, ae, ne, jf, Qr, vT, NE, nO, Y, Lf, iU, r, lo, en, bu, cl, t, iK, vk, ck, sS, cdd, kP, Dy, nM, iM, QG, au, im, zu, cdb, bt, rA, hS, On, gx, gV, kh, Gl, id, Mb, atY, iW, jh, KD, bS, Gn, jE, eq, Fc, iJ, ad, kL, hX, qC, av, Tg, hd, F, ib, cdc, bjn, Db, iC, lC, hP, bwV, wm, fb, nJ, WW, d, BN, BO, hG, qt, je, ff, eH, yy, aeV, Ej, aF, iV, Z, iR, bz, es, gk, eU, RV, pc, zr, Ew, bE, amS, sG, Hs, QC, bC, Gu, aeH, X, rk, BA, pC); #c1(MDM2) c2(NP_001138812) c3(E3 ubiquitin-protein ligase Mdm2 isoform i) c4(57867, 57868, 57869) c5(57870, 57871, 57872, 57873, 57874, 57875, 57876, 57877, 57878, 57879, 57880, 57881, 57882, 57883, 57884, 57885, 57886, 57887, 57888, 57889, 57890, 57891, 57892, 57893, 57894, 57895, 57896, 57897, 57898, 57899, 57900, 57901, 57902, 57903) c6( ) c7(T, aC, aiH, jW, fR, pf, hV, ea, if, al, cdf, bB, fd, qj, ci, aJa, ip, x, bv, bHS, gJ, aE, Bc, aeO, Qk, ajv, ja, cda, bnt, K, hu, pk, eL, cp, eG, zQ, aW, aEl, Ls, az, hl, QB, CT, kt, Qm, g, as, eF, ve, cm, bw, ccZ, iP, y, fT, eM, nB, AM, iA, iT, vA, cj, PV, k, m, cE, iu, alM, dD, gU, Qj, wu, sw, iB, ht, qy, n, fr, ct, Qh, qN, cde, co, Bl, jz, apS, Bj, aeX, bUP, h, eV, dL, Gw, Ha, dx, pl, cY, awV, FZ, TH, hQ, GA, aq, kp, yq, pm, mo, iN, Zj, op, oU, afb, ar, kT, iO, YE, bP, bbz, dB, hk, eD, Pl, Qu, Vy, hL, hj, at, MW, azr, Qz, WL, Pr, B, it, fM, dA, yk, eX, aA, hy, C, bX, Qo, il, lu, q, eA, eJ, ccY, amg, lg, hW, nv, qK, yg, aT, aHs, ae, ne, jf, Ar, vT, NE, nO, Y, Lf, iU, r, lo, en, bu, cl, t, iK, vk, ck, sS, cdd, kP, Dy, nM, iM, QG, au, im, zu, cdb, bt, rA, hS, On, gx, gV, kh, GI, id, Mb, atY, iW, jh, KO, bS, Gn, jE, eq, Fc, iJ, ad, KL, hX, qC, av, Tg, hd, F, ib, cdc, bjn, Db, iC, lC, hP, bwV, wm, fb, nJ, WW, d, BN, BO, hG, qt, je, ff, eH, yy, aeV, Ej, aF, iV, Z, iR, bz, es, gk, eL, RV, pc, zr, Ew, bE, amS, sG, Hs, QC, bC, Gu, aeH, X, rk, BA, pC); #c1(MDM2) c2(NP_001265391) c3(E3 ubiquitin-protein ligase Mdm2 isoform 1) c4(98455, 98456, 98457) c5(98458, 98459, 98460, 98461, 98462, 98463, 98464, 98465, 98466, 98467, 98468, 98469, 98470, 98471, 98472, 98473, 98474, 98475, 98476, 98477, 98478, 98479, 98480, 98481, 98482, 98483) c6( ) c7(T, aC, aiH, jW, fR, pf, hV, ea, if, al, cdf, bB, fd, qj, ci, aJa, ip, x, bv, bHS, gJ, aE, Bc, aeO, Qk, ajv, ja, cda, bnt, K, hu, pk, eL, cp, eG, zQ, aW, aEl, Ls, az, hl, QB, CT, kt, Qm, g, as, eF, ve, cm, bw, ccZ, iP, y, fT, eM, nB, AM, iA, iT, vA, cj, PV, k, m, cE, iu, alM, dD, gU, Qj, wu, sw, iB, ht, qy, n, fr, ct, Qh, qN, cde, co, Bl, jz, apS, Bj, aeX, bUP, h, eV, dL, Gw, Ha, dx, pl, cY, awV, FZ, TH, hQ, GA, aq, kp, yq, pm, mo, iN, Zj, op, oU, afb, ar, kT, iO, YE, bP, bbz, dB, hk, eD, Pl, Qu, Vy, hL, hj, at, MW, azr, Qz, WL, Pr, B, it, fM, dA, yk, eX, aA, hy, C, bX, Qo, il, lu, q, eA, eJ, ccY, amg, lg, hW, nv, qK, yg, aT, aHs, ae, ne, jf, Qr, vT, NE, nO, Y, Lf, iU, r, lo, en, bu, cl, t, iK, vk, ck, sS, cdd, KP, Dy, nM, iM, QG, au, im, zu, cdb, bt, rA, hS, On, gx, gV, kh, Gl, id, Mb, atY, iW, jh, KO, bS, Gn, jE, eq, Fc, iJ, ad, kL, hX, qC, av, Tg, hd, F, ib, cdc, bjn, Db, iC, lC, hP, bwV, wm, fb, nJ, WW, d, BN, BO, hG, qt, je, ff, eH, yy, aeV, Ej, aF, iV, Z, iR, bz, es, gk, eL, RV, pc, zr, Ew, bE, amS, sG, Hs, QC, bC, Gu, aeH, X, rk, BA, pC); #c1(MDM2) c2(NP_002383) c3(E3 ubiquitin-protein ligase Mdm2 isoform a) c4(128001, 128002, 128003, 128004, 128005) c5(128006, 128007, 128008, 128009, 128010, 128011, 128012, 128013, 128014, 128015, 128016, 128017, 128018, 128019) c6( ) c7(T, aC, aiH, jW, fR, pf, hV, ea, if, al, cdf, bB, fd, qj, ci, aJa, ip, x, bv, bHS, gJ, aE, Bc, aeO, Qk, ajv, ja, cda, bnt, K, hu, pk, eL, cp, eG, zQ, aW, aEl, Ls, az, hl, QB, CT, kt, Qm, g, as, ef, ve, cm, bw, ccZ, iP, y, fT, eM, nB, AM, iA, iT, vA, cj, PV, k, m, cE, iu, alM, dD, gU, Qj, wu, sw, iB, ht, qy, n, fr, ct, Qh, qN, cde, co, Bl, jz, apS, Bj, aeX, bUP, h, eV, dL, Gw, Ha, dx, pl, cY, awV, FZ, TH, hQ, GA, aq, kp, yq, pm, mo, iN, Zj, op, oU, afb, ar, kT, iO, YE, bP, bbz, dB, hk, eD, Pl, Qu, Vy, hL, hj, at, MW, azr, Qz, WL, Pr, B, it, fM, dA, yk, eX, aA, hy, C, bX, Qo, il, lu, q, eA, eJ, ccY, amg, lg, hW, nv, qK, yg, aT, aHs, ae, ne, jf, Qr, vT, NE, nO, Y, Lf, iU, r, lo, en, bu, cl, t, iK, vk, ck, sS, cdd, kP, Dy, nM, iM, QG, au, im, zu, cdb, bt, rA, hS, On, gx, gV, kh, Gl, id, Mb, atY, iW, jh, KO, bS, Gn, jE, eq, Fc, iJ, ad, KL, hX, qC, av, Tg, hd, F, ib, cdc, bjn, Db, iC, lC, hP, bwV, wm, fb, nJ, WW, d, BN, BO, hG, qt, je, ff, eH, yy, aeV, Ej, aF, iV, Z, iR, bz, es, gk, eL, RV, pc, zr, Ew, bE, amS, sG, Hs, QC, bC, Gu, aeH, X, rk, BA, pC); #c1(MDM2) c2(XP_005268929) c3(E3 ubiquitin-protein ligase Mdm2 isoform X1) c4( ) c5(282197, 282198, 282199, 282200, 282201, 282202, 282203, 282204, 282205, 282206, 282207, 282208, 282209, 282210, 282211, 282212) c6( ) c7(T, aC, aiH, jW, fR, pf, hV, ea, if, al, cdf, bB, fd, qj, ci, aJa, ip, x, bv, bHS, gJ, aE, Bc, aeO, Qk, ajv, ja, cda, bnt, K, hu, pk, eL, cp, eG, zQ, aW, aEl, Ls, az, hl, QB, CT, kt, Qm, g, as, eF, ve, cm, bw, ccZ, iP, y, fT, eM, nB, AM, iA, iT, vA, cj, PV, k, m, cE, iu, alM, dD, gU, Qj, wu, sw, iB, ht, qy, n, fr, ct, Qh, qN, cde, co, Bl, jz, apS, Bj, aeX, bUP, h, eV, dL, Gw, Ha, dx, pl, cY, awV, FZ, TH, hQ, GA, aq, kp, yq, pm, mo, iN, Zj, op, ol, afb, ar, kT, iO, YE, bP, bbz, dB, hk, eD, Pl, Qu, Vy, hL, hj, at, MW, azr, Qz, WL, Pr, B, it, fM, dA, yk, eX, aA, hy, C, bX, Qo, il, lu, q, eA, eJ, ccY, amg, lg, hW, nv, qK, yg, aT, aHs, ae, ne, jf, Qr, vT, NE, nO, Y, Lf, iU, r, lo, en, bu, cl, t, iK, vk, ck, sS, cdd, kP, Dy, nM, iM, QG, au, im, zu, cdb, bt, rA, hS, On, gx, gV, kh, Gl, id, Mb, atY, iW, jh, KO, bS, Gn, jE, eq, Fc, iJ, ad, kL, hX, qC, av, Tg, hd, F, ib, cdc, bjn, Db, iC, lC, hP, bwV, wm, fb, nJ, WW, d, BN, BO, hG, qt, je, ff, eH, yy, aeV, Ej, aF, iV, Z, iR, bz, es, gk, eU, RV, pc, zr, Ew, bE, amS, sG, Hs, QC, bC, Gu, aeH, X, rk, BA, pC); #c1(MDM2) c2(XP_006719462) c3(E3 ubiquitin-protein ligase Mdm2 isoform X2) c4(292492) c5( ) c6( ) c7(T, aC, aiH, jW, fR, pf, hV, ea, if, al, cdf, bB, fd, qj, ci, aJa, ip, x, bv, bHS, gJ, aE, Bc, aeO, Qk, ajv, ja, cda, bnt, K, hu, pk, eL, cp, eG, zQ, aW, aEl, Ls, az, hl, QB, CT, kt, Qm, g, as, eF, ve, cm, bw, ccZ, iP, y, fT, eM, nB, AM, iA, iT, vA, cj, PV, k, m, cE, iu, alM, dD, gU, Qj, wu, sw, iB, ht, qy, n, fr, ct, Qh, qN, cde, co, Bl, jz, apS, Bj, aeX, bUP, h, eV, dL, Gw, Ha, dx, pl, cY, awV, FZ, TH, hQ, GA, aq, kp, yq, pm, mo, iN, Zj, op, oU, afb, ar, KT, iO, YE, bP, bbz, dB, hk, eD, Pl, Qu, Vy, hL, hj, at, MW, azr, Qz, WL, Pr, B, it, fM, dA, yk, eX, aA, hy, C, bX, Qo, il, lu, q, eA, eJ, ccY, amg, lg, hW, nv, qK, yg, aT, aHs, ae, ne, jf, Qr, vT, NE, nO, Y, Lf, iU, r, lo, en, bu, cl, t, iK, vk, ck, sS, cdd, kP, Dy, nM, iM, QG, au, im, zu, cdb, bt, rA, hS, On, gx, gV, kh, Gl, id, Mb, atY, iW, jh, KO, bS, Gn, jE, eq, Fc, iJ, ad, kL, hX, qC, av, Tg, hd, F, ib, cdc, bjn, Db, iC, lC, hP, bwV, wm, fb, nJ, WW, d, BN, BO, hG, qt, je, ff, eH, yy, aeV, Ej, aF, iV, Z, iR, bz, es, gk, eU, RV, pc, zr, Ew, bE, amS, sG, Hs, QC, bC, Gu, aeH, X, rk, BA, pC); #c1(MDM2) c2(XP_006719463) c3(E3 ubiquitin-protein ligase Mdm2 isoform X3) c4(292493) c5( ) c6( ) c7(T, aC, aiH, jW, fR, pf, hV, ea, if, al, cdf, bB, fd, qj, ci, aJa, ip, x, bv, bHS, gJ, aE, Bc, aeO, Ok, ajv, ja, cda, bnt, K, hu, pk, eL, cp, eG, zQ, aW, aEl, Ls, az, hl, QB, CT, kt, Qm, g, as, ef, ve, cm, bw, ccZ, iP, y, fT, eM, nB, AM, iA, lT, vA, cj, PV, k, m, cE, iu, alM, dD, gU, Qj, wu, sw, iB, ht, qy, n, fr, ct, Qh, qN, cde, co, Bl, jz, apS, Bj, aeX, bUP, h, eV, dL, Gw, Ha, dx, pl, cY, awV, FZ, TH, hQ, GA, aq, kp, yq, pm, mo, iN, Zj, op, oU, afb, ar, KT, iO, YE, bP, bbz, dB, hk, eD, Pl, Qu, Vy, hL, hj, at, MW, azr, Qz, WL, Pr, B, it, fM, dA, yk, eX, aA, hy, C, bX, Qo, il, lu, q, eA, eJ, ccY, amg, lg, hW, nv, qK, yg, aT, aHs, ae, ne, jf, Ar, vT, NE, nO, Y, Lf, iU, r, lo, en, bu, cl, t, iK, vk, ck, sS, cdd, kP, Dy, nM, iM, QG, au, im, zu, cdb, bt, rA, hS, On, gx, gV, kh, Gl, id, Mb, atY, iW, jh, KO, bS, Gn, jE, eq, Fc, iJ, ad, kL, hX, qC, av, Tg, hd, F, ib, cdc, bjn, Db, iC, lC, hP, bwV, wm, fb, nJ, WW, d, BN, BO, hG, qt, je, ff, eH, yy, aeV, Ej, aF, iV, Z, iR, bz, es, gk, eU, RV, pc, zr, Ew, bE, amS, sG, Hs, QC, bC, Gu, aeH, X, rk, BA, pC); #c1(MDM4) c2(NP_001191100) c3(protein Mdm4 isoform 2) c4(80980, 80981) c5(80982, 80983, 80984, 80985, 80986, 80987, 80988, 80989, 80990, 80991, 80992, 80993) c6( ) c7(T, bO, fd, bB, ci, x, bv, aE, K, pk, bc, cp, aW, g, as, eF, kJ, ir, cm, eM, nB, cj, m, k, gn, ik, iB, n, fr, ft, kl, kH, h, dl, ia, hQ, GA, aq, bP, dB, hR, MW, it, B, aA, hy, j, q, er, eA, cF, aT, ae, iU, Y, ev, r, en, t, iK, hY, ck, iM, kP, Dy, ac, au, bt, ao, kG, hS, ha, jh, hF, iJ, ad, hd, ib, d, BN, je, ff, eH, KD, aF, bz, bE, ne); #c1(MDM4) c2(NP_001191101) c3(protein Mdm4 isoform 3) c4(80994) c5(80995, 80996, 80997, 80998, 80999, 81000) c6( ) c7(T, bO, fd, bB, ci, x, bv, aE, K, pk, bc, cp, aW, g, as, eF, kJ, ir, cm, eM, nB, cj, m, k, gn, ik, iB, n, fr, ft, kl, kH, h, dl, ia, hQ, GA, aq, bP, dB, hR, MW, it, B, aA, hy, j, q, er, eA, cF, aT, ae, iU, Y, ev, r, en, t, iK, hY, ck, iM, kP, Dy, ac, au, bt, ao, kG, hS, ha, jh, hF, iJ, ad, hd, ib, d, BN, je, ff, eH, kD, aF, bz, bE, ne); #c1(MDM4) c2(NP_001265446) c3(protein Mdm4 isoform 5) c4( ) c5(98601, 98602, 98603, 98604, 98605, 98606) c6( ) c7(T, bO, fd, bB, ci, x, bv, aE, K, pk, bc, cp, aW, g, as, eF, kJ, ir, cm, eM, nB, cj, m, k, gn, ik, iB, n, fr, ft, kl, kH, h, dL, ia, hQ, GA, aq, bP, dB, hR, MW, it, B, aA, hy, j, q, er, eA, cF, aT, ae, iU, Y, ev, r, en, t, iK, hY, ck, iM, kP, Dy, ac, au, bt, ao, kG, hS, ha, jh, hF, iJ, ad, hd, ib, d, BN, je, ff, eH, kD, aF, bz, bE, ne); #c1(MDM4) c2(NP_002384) c3(protein Mdm4 isoform 1) c4(128020, 128021) c5(128022, 128023, 128024, 128025, 128026, 128027, 128028, 128029) c6( ) c7(T, bO, fd, bB, ci, x, bv, aE, K, pk, bc, cp, aW, g, as, eF, kJ, ir, cm, eM, nB, cj, m, k, gn, ik, iB, n, fr, ft, kl, kH, h, dL, ia, hQ, GA, aq, bP, dB, hR, MW, it, B, aA, hy, j, q, er, eA, cF, aT, ae, iU, Y, ev, r, en, t, iK, hY, ck, iM, KP, Dy, ac, au, bt, ao, kG, hS, ha, jh, hF, iJ, ad, hd, ib, d, BN, je, ff, eH, KD, aF, bz, bE, ne); #c1(MDM4) c2(XP_011507867) c3(protein Mdm4 isoform X3) c4(298043) c5( ) c6( ) c7(T, bO, fd, bB, ci, x, bv, aE, K, pk, bc, cp, aW, g, as, eF, kJ, ir, cm, eM, nB, cj, m, k, gn, ik, iB, n, fr, ft, kl, kH, h, dl, ia, hQ, GA, aq, bP, dB, hR, MW, it, B, aA, hy, j, q, er, eA, cF, aT, ae, iU, Y, ev, r, en, t, iK, hY, ck, iM, kP, Dy, ac, au, bt, ao, kG, hS, ha, jh, hF, iJ, ad, hd, ib, d, BN, je, ff, eH, kD, aF, bz, bE, ne); #c1(MDM4) c2(XP_011507868) c3(protein Mdm4 isoform X4) c4(298044) c5(298045, 298046, 298047, 298048, 298049, 298050, 298051, 298052, 298053, 298054, 298055) c6( ) c7(T, bO, fd, bB, ci, x, bv, aE, K, pk, bc, cp, aW, g, as, eF, kJ, ir, cm, eM, nB, cj, m, k, gn, ik, iB, n, fr, ft, kl, kH, h, dl, ia, hQ, GA, aq, bP, dB, hR, MW, it, B, aA, hy, j, q, er, eA, cF, aT, ae, iU, Y, ev, r, en, t, iK, hY, ck, iM, kP, Dy, ac, au, bt, ao, kG, hS, ha, jh, hF, iJ, ad, hd, ib, d, BN, je, ff, eH, KD, aF, bz, bE, ne); #c1(MDM4) c2(XP_011507869) c3(protein Mdm4 isoform X5) c4(298056) c5(298057, 298058, 298059, 298060, 298061, 298062) c6( ) c7(T, bO, fd, bB, ci, x, bv, aE, K, pk, bc, cp, aW, g, as, eF, kJ, ir, cm, eM, nB, cj, m, k, gn, ik, iB, n, fr, ft, kl, kH, h, dl, ia, hQ, GA, aq, bP, dB, hR, MW, it, B, aA, hy, j, q, er, eA, cF, aT, ae, iU, Y, ev, r, en, t, iK, hY, ck, iM, kP, Dy, ac, au, bt, ao, kG, hS, ha, jh, hF, iJ, ad, hd, ib, d, BN, je, ff, eH, KD, aF, bz, bE, ne); #c1(MDM4) c2(XP_011507870) c3(protein Mdm4 isoform X1) c4(298063) c5(298064, 298065, 298066, 298067, 298068) c6( ) c7(T, bO, fd, bB, ci, x, bv, aE, K, pk, bc, cp, aW, g, as, eF, kJ, ir, cm, eM, nB, cj, m, k, gn, ik, iB, n, fr, ft, kl, kH, h, dL, ia, hQ, GA, aq, bP, dB, hR, MW, it, B, aA, hy, j, q, er, eA, cF, aT, ae, iU, Y, ev, r, en, t, iK, hY, ck, iM, kP, Dy, ac, au, bt, ao, kG, hS, ha, jh, hF, iJ, ad, hd, ib, d, BN, je, ff, eH, kD, aF, bz, bE, ne); #c1(ME1) c2(NP_002386) c3(NADP-dependent malic enzyme) c4(128037, 128038) c5(128039, 128040, 128041, 128042, 128043, 128044, 128045) c6( ) c7(bL, ae, ix, d, eM, Qu, aW, Y, ff, aT, fe, ail, v, eA, en, as, bz, B, ad); #c1(MEI) c2(XP_011534138) c3(NADP-dependent malic enzyme isoform X1) c4(330417, 330418) c5(330419, 330420, 330421, 330422, 330423, 330424, 330425, 330426, 330427, 330428, 330429, 330430, 330431, 330432, 330433, 330434, 330435) c6( ) c7(bL, ae, ix, d, eM, Qu, aW, Y, ff, aT, fe, ail, v, eA, en, as, bz, B, ad); #c1(ME2) c2(NP_001161807) c3(NAD-dependent malic enzyme, mitochondrial isoform 2 precursor) c4(66043) c5(66044, 66045, 66046, 66047, 66048, 66049) c6( ) c7(ck, Cq, VR, Y, fe, gl, iD, bK, hg, ey, as, ae, ad); #c1(ME2) c2(NP_002387) c3(NAD-dependent malic enzyme, mitochondrial isoform 1 precursor) c4(128046) c5(128047, 128048, 128049, 128050, 128051, 128052) c6( ) c7(ck, Cq, VR, Y, fe, gl, iD, bK, hg, ey, as, ae, ad); #c1(ME3) c2(NP_001155058) c3(NADP-dependent malic enzyme, mitochondrial) c4(61398, 61399, 61400, 61401, 61402, 61403, 61404, 61405) c5(61406, 61407, 61408, 61409, 61410, 61411, 61412, 61413, 61414, 61415, 61416, 61417, 61418, 61419, 61420, 61421, 61422, 61423, 61424, 61425) c6( ) c7(fe, Y, ae, ad, as); #c1(ME3) c2(XP_011543037) c3(NADP-dependent malic enzyme, mitochondrial isoform X2) c4(341783) c5(341784, 341785, 341786, 341787, 341788, 341789) c6( ) c7(fe, Y, ae, ad, as); #c1(ME3) c2(XP_011543038) c3(NADP-dependent malic enzyme, mitochondrial isoform X3) c4( ) c5(341790, 341791, 341792, 341793, 341794, 341795, 341796, 341797, 341798, 341799, 341800, 341801, 341802, 341803, 341804, 341805, 341806, 341807, 341808, 341809, 341810, 341811, 341812, 341813, 341814, 341815, 341816, 341817, 341818, 341819, 341820, 341821, 341822, 341823, 341824, 341825) c6( ) c7(fe, Y, ae, ad, as); #c1(MEA1) c2(NP_055438) c3(male-enhanced antigen 1) c4(181133) c5(181134, 181135, 181136, 181137, 181138, 181139) c6( ) c7(nv, T); #c1(MECOM) c2(NP_001098547) c3(MDS1 and EV11 complex locus protein EV11 isoform a) c4(41358) c5(41359, 41360, 41361, 41362, 41363, 41364, 41365, 41366, 41367, 41368, 41369, 41370, 41371, 41372, 41373) c6( ) c7(T, os, gp, oe, oA, bs, bB, it, oZ, hF, pd, q, br, cd, eA, iC, x, pN, aEX, oW, aT, h, dz, hn, K, jj, pq, iU, et, iZ, aW, ff, gK, r, kp, en, cl, pm, t, bw, ck, ou, bP, c, eM, bE, iT, eB, cj, wR, dW, oX, m, k, bt, KS, X); #c1(MECOM) c2(NP_001157471) c3(MDS1 and EV11 complex locus protein EV11 isoform d) c4(62230, 62231) c5( ) c6( ) c7(T, os, gp, oe, oA, bs, bB, it, oZ, hF, pd, q, br, cd, eA, iC, x, pN, aEX, oW, aT, h, dz, hn, K, jj, pq, iU, et, iZ, aW, ff, gK, r, kp, en, cl, pm, t, bw, ck, ou, bP, c, eM, bE, iT, eB, cj, wR, dW, oX, m, k, bt, kS, X); #c1(MECOM) c2(NP_001157472) c3(MDS1 and EV11 complex locus protein EV11 isoform e) c4(62232, 62233, 62234, 62235) c5(62236, 62237, 62238, 62239, 62240, 62241, 62242, 62243, 62244, 62245) c6( ) c7(T, os, gp, oe, oA, bs, bB, it, oZ, hF, pd, q, br, cd, eA, iC, x, pN, aEX, oW, aT, h, dz, hn, K, jj, pq, iU, et, iZ, aW, ff, gK, r, kp, en, cl, pm, t, bw, ck, ou, bP, c, eM, bE, lT, eB, cj, wR, dW, oX, m, k, bt, kS, X); #c1(MECOM) c2(NP_001192123) c3(MDS1 and EV11 complex locus protein EV11 isoform b) c4(81924) c5(81925, 81926, 81927, 81928, 81929, 81930, 81931, 81932) c6( ) c7(T, os, gp, oe, oA, bs, bB, it, oZ, hF, pd, q, br, cd, eA, iC, x, pN, aEX, oW, aT, h, dz, hn, K, jj, pq, iU, et, iZ, aW, ff, gK, r, kp, en, cl, pm, t, bw, ck, ou, bP, c, eM, bE, iT, eB, cj, wR, dW, oX, m, k, bt, kS, X); #c1(MECOM) c2(NP_004982) c3(MDS1 and EV11 complex locus protein EV11 isoform c) c4(151592) c5(151593, 151594, 151595) c6( ) c7(T, os, gp, oe, oA, bs, bB, it, oZ, hF, pd, q, br, cd, eA, iC, x, pN, aEX, oW, aT, h, dz, hn, K, jj, pq, iU, et, iZ, aW, ff, gK, r, kp, en, cl, pm, t, bw, ck, ou, bP, c, eM, bE, iT, eB, cj, wR, dW, oX, m, k, bt, kS, X); #c1(MECOM) c2(XP_005247270) c3(MDS1 and EV11 complex locus protein EV11 isoform X3) c4( ) c5(267698, 267699, 267700, 267701, 267702, 267703, 267704, 267705, 267706, 267707, 267708, 267709) c6( ) c7(T, os, gp, oe, oA, bs, bB, it, oZ, hF, pd, q, br, cd, eA, iC, x, pN, aEX, oW, aT, h, dz, hn, K, jj, pq, iU, et, iZ, aW, ff, gK, r, kp, en, cl, pm, t, bw, ck, ou, bP, c, eM, bE, iT, eB, cj, wR, dW, oX, m, k, bt, KS, X); #c1(MECOM) c2(XP_005247272) c3(MDS1 and EV11 complex locus protein EV11 isoform X5) c4(267710) c5(267711, 267712, 267713, 267714, 267715, 267716, 267717, 267718) c6( ) c7(T, os, gp, oe, oA, bs, bB, it, oZ, hF, pd, q, br, cd, eA, iC, x, pN, aEX, oW, aT, h, dz, hn, K, jj, pq, iU, et, iZ, aW, ff, gK, r, kp, en, cl, pm, t, bw, ck, ou, bP, c, eM, bE, iT, eB, cj, wR, dW, oX, m, k, bt, kS, X); #c1(MECOM) c2(XP_005247283) c3(MDS1 and EV11 complex locus protein EV11 isoform X10) c4( ) c5(267719, 267720, 267721, 267722, 267723, 267724, 267725, 267726, 267727, 267728, 267729, 267730, 267731, 267732, 267733, 267734, 267735, 267736, 267737, 267738, 267739, 267740, 267741, 267742, 267743, 267744) c6( ) c7(T, os, gp, oe, oA, bs, bB, it, oZ, hF, pd, q, br, cd, eA, iC, x, pN, aEX, oW, aT, h, dz, hn, K, jj, pq, iU, et, iZ, aW, ff, gK, r, kp, en, cl, pm, t, bw, ck, ou, bP, c, eM, bE, iT, eB, cj, wR, dW, oX, m, k, bt, kS, X); #c1(MECOM) c2(XP_011510848) c3(MDS1 and EV11 complex locus protein EV11 isoform X6) c4(301303) c5(301304) c6( ) c7(T, os, gp, de, DA, bs, bB, it, oZ, hF, pd, q, br, cd, eA, iC, x, pN, aEX, oW, aT, h, dz, hn, K, jj, pq, iU, et, iZ, aW, ff, gK, r, kp, en, cl, pm, t, bw, ck, ou, bP, c, eM, bE, iT, eB, cj, wR, dW, oX, m, k, bt, KS, X); #c1(MECP2) c2(NP_001104262) c3(methyl-CpG-binding protein 2 isoform 2) c4(42083) c5(42084, 42085, 42086, 42087, 42088) c6( ) c7(T, ahc, fd, amj, aR, dE, aVA, dV, aU, amA, aVB, aW, Az, apn, vs, Ku, cm, eM, cdi, lq, cdm, cdn, Kk, n, ct, nA, rc, bdO, ask, he, on, cC, Fn, Cw, QK, hq, cdg, bvv, agl, fL, Pr, B, fG, Nt, aA, il, q, eJ, MR, hb, jZ, aT, rz, ae, afy, NE, oV, r, cu, bu, bQ, f, xw, pb, bN, Qi, cdk, wR, Fl, id, ha, cdh, cdj, bS, mC, xt, ad, hX, av, l, afm, nd, lm, cx, aeQ, aqa, alW, VX, eZ, nf, aF, ys, Z, Kp, bz, dr, all, LB, Bv, agX, pk, y, cdl, jr); #c1(MECP2) c2(NP_004983) c3(methyl-CpG-binding protein 2 isoform 1) c4(151596, 151597, 151598, 151599) c5(151600, 151601, 151602, 151603, 151604, 151605, 151606, 151607, 151608, 151609, 151610, 151611, 151612, 151613, 151614, 151615, 151616, 151617, 151618, 151619, 151620, 151621, 151622, 151623, 151624, 151625) c6( ) c7(T, ahc, fd, amj, aR, dE, aVA, dV, aU, amA, aVB, aW, Az, apn, vs, Ku, cm, eM, cdi, lq, cdm, cdn, Kk, n, ct, nA, rc, bdO, ask, he, on, cC, Fn, Cw, QK, hq, cdg, bvv, agl, fL, Pr, B, fG, Nt, aA, il, q, eJ, MR, hb, jZ, aT, rz, ae, afy, NE, oV, r, cu, bu, bQ, f, xw, pb, bN, Qi, cdk, wR, Fl, id, ha, cdh, cdj, bS, mC, xt, ad, hX, av, l, afm, nd, lm, cx, aeQ, aqa, alW, VX, eZ, nf, aF, ys, Z, Kp, bz, dr, all, LB, Bv, agX, pk, y, cdl, jr); #c1(MED1) c2(NP_004765) c3(mediator of RNA polymerase Il transcription subunit 1) c4(149408, 149409) c5(149410, 149411, 149412, 149413, 149414, 149415, 149416) c6( ) c7(T, ha, aA, bB, iJ, q, ad, aDF, dP, iu, aE, ds, aT, ae, dx, jf, iF, aW, Y, r, aF, g, t, axp, eM, mg, m, bt, B); #c1(MED1) c2(XP_006722020) c3(mediator of RNA polymerase II transcription subunit 1 isoform X2) c4( ) c5(294553, 294554, 294555, 294556, 294557, 294558) c6( ) c7(T, ha, aA, bB, iJ, q, ad, aDF, dP, iu, aE, ds, aT, ae, dx, jf, iF, aW, Y, r, aF, g, t, axp, eM, mg, m, bt, B); #c1(MED(2) c2(NP_005111) c3(mediator of RNA polymerase II transcription subunit 12) c4(152972) c5(152973, 152974, 152975, 152976, 152977, 152978, 152979, 152980, 152981, 152982, 152983, 152984, 152985) c6( ) c7(HQ, cZ, T, bO, cz, Bm, bD, aNG, bS, fr, ct, nA, bgR, q, aR, cd, ad, Zp, lS, alD, cdt, amg, aV, HJ, hW, oj, aT, iv, Th, bK, K, vd, bU, dd, he, cdp, ae, af, awL, aAe, Dz, nP, cdr, dH, mA, ff, cdq, nf, gl, cds, r, en, dw, Z, kC, op, bN, bUr, cdo, QG, ac, c, eM, bE, aps, mG, vA, U, awk, acJ, eQ, lM, gT, B, Al); #c1(MED12) c2(XP_005262374) c3(mediator of RNA polymerase II transcription subunit 12 isoform X1) c4(277867, 277868, 277869, 277870) c5(277871, 277872, 277873, 277874, 277875, 277876, 277877, 277878, 277879, 277880, 277881, 277882, 277883, 277884, 277885, 277886, 277887, 277888) c6( ) c7(HQ, cZ, T, bO, cz, Bm, bD, aNG, bS, fr, ct, nA, bgR, q, aR, cd, ad, Zp, IS, alD, cdt, amg, aV, HJ, hW, oj, aT, iv, Th, bK, K, vd, bU, dd, he, cdp, ae, af, awl, aAe, Dz, nP, cdr, dH, mA, ff, cdq, nf, gl, cds, r, en, dw, Z, kC, op, bN, bUr, cdo, QG, ac, c, eM, bE, aps, mG, vA, U, awk, acJ, eL, lM, gT, B, Al); #c1(MED(2) c2(XP_005262376) c3(mediator of RNA polymerase II transcription subunit 12 isoform X2) c4(277889, 277890, 277891) c5(277892, 277893, 277894, 277895, 277896, 277897, 277898, 277899, 277900, 277901, 277902, 277903, 277904, 277905, 277906, 277907, 277908, 277909) c6( ) c7(HQ, cZ, T, bO, cz, Bm, bD, aNG, bS, fr, ct, nA, bgR, q, aR, cd, ad, Zp, lS, alD, cdt, amg, aV, HJ, hW, oj, aT, iv, Th, bK, K, vd, bU, dd, he, cdp, ae, af, awL, aAe, Dz, nP, cdr, dH, mA, ff, cdq, nf, gl, cds, r, en, dw, Z, kC, op, bN, bur, cdo, QG, ac, c, eM, bE, aps, mG, vA, U, awK, acJ, eL, lM, gT, B, Al); #c1(MED12L) c2(NP_443728) c3(mediator of RNA polymerase II transcription subunit 12-like protein) c4(230035) c5(230036, 230037, 230038, 230039, 230040, 230041, 230042, 230043, 230044, 230045, 230046, 230047) c6( ) c7(cm, oe, oc, cz); #c1(MED12L) c2(XP_006713550) c3(mediator of RNA polymerase II transcription subunit 12-like protein isoform X4) c4(287230) c5( ) c6( ) c7(cm, de, oc, cz); #c1(MED12L) c2(XP_011510691) c3(mediator of RNA polymerase II transcription subunit 12-like protein isoform X6) c4(301135) c5( ) c6( ) c7(cm, oe, oc, cz); #c1(MED12L) c2(XP_011510701) c3(mediator of RNA polymerase II transcription subunit 12-like protein isoform X15) c4( ) c5(301136, 301137, 301138, 301139, 301140, 301141) c6( ) c7(cm, oe, oc, cz); #c1(MED13) c2(NP_005112) c3(mediator of RNA polymerase II transcription subunit 13) c4(152986, 152987) c5(152988, 152989, 152990, 152991, 152992, 152993, 152994, 152995, 152996, 152997, 152998, 152999) c6( ) c7(om, vA, dq, v, K, bD, op); #c1(MED13L) c2(NP_056150) c3(mediator of RNA polymerase II transcription subunit 13-like) c4(185969) c5(185970, 185971, 185972, 185973, 185974, 185975) c6( ) c7(bD, alo, nA, acl); #c1(MED14) c2(NP_004220) c3(mediator of RNA polymerase II transcription subunit 14) c4(144399) c5(144400, 144401, 144402, 144403, 144404, 144405) c6( ) c7(dP, aW, eZ, h, aT); #c1(MED14) c2(XP_011542302) c3(mediator of RNA polymerase II transcription subunit 14 isoform X1) c4(340895, 340896) c5(340897, 340898, 340899, 340900, 340901, 340902, 340903, 340904, 340905, 340906, 340907, 340908) c6( ) c7(dP, aW, eZ, h, aT); #c1(MED15) c2(NP_001003891) c3(mediator of RNA polymerase II transcription subunit 15 isoform a) c4(20241) c5(20242) c6( ) c7(ck, bF, T, agd, jz, lU, MD, bE, RX, cr, it, bB, ci, cl, lk, ae, B, ad); #c1(MED15) c2(NP_001280165) c3(mediator of RNA polymerase II transcription subunit 15 isoform e) c4(111464, 111465, 111466) c5( ) c6( ) c7(ck, bF, T, agd, jz, lU, MD, bE, RX, cr, it, bB, ci, cl, lk, ae, B, ad); #c1(MED15) c2(NP_056973) c3(mediator of RNA polymerase II transcription subunit 15 isoform b) c4(188281, 188282) c5( ) c6( ) c7(ck, bF, T, agd, jz, lU, MD, bE, RX, cr, it, bB, ci, cl, lk, ae, B, ad); #c1(MED15) c2(XP_006724326) c3(mediator of RNA polymerase II transcription subunit 15 isoform X3) c4(295944) c5( ) c6( ) c7(ck, bF, T, agd, jz, lU, MD, bE, RX, cr, it, bB, ci, cl, lk, ae, B, ad); #c1(MED15) c2(XP_011528518) c3(mediator of RNA polymerase II transcription subunit 15 isoform X4) c4( ) c5(323617, 323618, 323619, 323620, 323621, 323622) c6( ) c7(ck, bF, T, agd, jz, lU, MD, bE, RX, cr, it, bB, ci, cl, lk, ae, B, ad); #c1(MED15) c2(XP_011528522) c3(mediator of RNA polymerase II transcription subunit 15 isoform X7) c4( ) c5(323623, 323624, 323625, 323626, 323627, 323628, 323629, 323630) c6( ) c7(ck, bF, T, agd, jz, lU, MD, bE, RX, cr, it, bB, ci, cl, lk, ae, B, ad); #c1(MED15) c2(XP_011528523) c3(mediator of RNA polymerase II transcription subunit 15 isoform X8) c4( ) c5(323631, 323632, 323633, 323634, 323635, 323636, 323637, 323638, 323639, 323640, 323641, 323642, 323643, 323644, 323645, 323646, 323647, 323648, 323649, 323650, 323651, 323652, 323653, 323654, 323655, 323656, 323657, 323658, 323659, 323660, 323661, 323662, 323663, 323664) c6( ) c7(ck, bF, T, agd, jz, lU, MD, bE, RX, cr, it, bB, ci, cl, lk, ae, B, ad); #c1(MED16) c2(NP_005472) c3(mediator of RNA polymerase II transcription subunit 16) c4(156442, 156443, 156444, 156445, 156446, 156447, 156448, 156449, 156450) c5(156451, 156452, 156453, 156454, 156455, 156456, 156457, 156458, 156459, 156460, 156461, 156462, 156463, 156464, 156465, 156466, 156467, 156468, 156469, 156470, 156471, 156472, 156473, 156474, 156475, 156476, 156477, 156478, 156479, 156480, 156481, 156482, 156483, 156484, 156485, 156486, 156487, 156488, 156489, 156490, 156491, 156492, 156493, 156494, 156495, 156496, 156497, 156498, 156499, 156500, 156501, 156502, 156503, 156504, 156505) c6( ) c7(dP); #c1(MED16) c2(XP_011525906) c3(mediator of RNA polymerase II transcription subunit 16 isoform X1) c4(320308) c5(320309, 320310, 320311, 320312, 320313, 320314) c6( ) c7(dP); #c1(MED17) c2(NP_004259) c3(mediator of RNA polymerase II transcription subunit (7) c4(144660, 144661) c5(144662, 144663, 144664, 144665, 144666, 144667, 144668, 144669, 144670, 144671, 144672, 144673, 144674, 144675, 144676, 144677, 144678, 144679) c6( ) c7(dP, cdu, ae, ad); #c1(MED(7) c2(XP_011541370) c3(mediator of RNA polymerase II transcription subunit 17 isoform X1) c4(339982) c5( ) c6( ) c7(dP, cdu, ae, ad); #c1(MED19) c2(NP_703151) c3(mediator of RNA polymerase II transcription subunit 19) c4( ) c5(246045, 246046, 246047, 246048, 246049, 246050) c6( ) c7(ae, T, eM, aW, eL, eH, a, dL, eF, bz, iu, ad); #c1(MED22) c2(NP_598395) c3(mediator of RNA polymerase II transcription subunit 22 isoform b) c4(235482, 235483, 235484, 235485) c5(235486, 235487, 235488, 235489, 235490, 235491, 235492, 235493, 235494, 235495) c6( ) c7(bM); #c1(MED22) c2(NP_852468) c3(mediator of RNA polymerase II transcription subunit 22 isoform c) c4(254958, 254959) c5(254960, 254961, 254962, 254963, 254964, 254965, 254966, 254967, 254968, 254969, 254970, 254971, 254972, 254973, 254974, 254975, 254976, 254977, 254978, 254979, 254980, 254981, 254982, 254983, 254984, 254985, 254986, 254987, 254988, 254989, 254990, 254991, 254992, 254993, 254994, 254995, 254996, 254997) c6( ) c7(bM); #c1(MED23) c2(NP_001257451) c3(mediator of RNA polymerase II transcription subunit 23 isoform d) c4(95429) c5(95430, 95431, 95432, 95433, 95434, 95435) c6( ) c7(eW, T, cdv, Y, cz, mw, mt, nA, it, cd, cp); #c1(MED23) c2(NP_004821) c3(mediator of RNA polymerase II transcription subunit 23 isoform a) c4(150054, 150055) c5(150056, 150057, 150058, 150059, 150060, 150061, 150062, 150063, 150064, 150065, 150066, 150067) c6( ) c7(eW, T, cdv, Y, cz, mw, mt, nA, it, cd, cp); #c1(MED23) c2(NP_057063) c3(mediator of RNA polymerase II transcription subunit 23 isoform b) c4(188687, 188688, 188689, 188690) c5(188691, 188692, 188693, 188694, 188695, 188696, 188697, 188698, 188699, 188700, 188701, 188702, 188703, 188704, 188705, 188706, 188707, 188708, 188709) c6( ) c7(eW, T, cdv, Y, cz, mw, mt, nA, it, cd, cp); #c1(MED23) c2(XP_005267280) c3(mediator of RNA polymerase II transcription subunit 23 isoform X1) c4(280849, 280850, 280851, 280852) c5(280853, 280854, 280855, 280856, 280857, 280858, 280859, 280860, 280861, 280862, 280863, 280864, 280865, 280866, 280867, 280868, 280869, 280870, 280871, 280872, 280873, 280874) c6( ) c7(eW, T, cdv, Y, cz, mw, mt, nA, it, cd, cp); #c1(MED23) c2(XP_006715675) c3(mediator of RNA polymerase II transcription subunit 23 isoform X3) c4( ) c5(289702, 289703, 289704, 289705, 289706, 289707, 289708, 289709, 289710, 289711, 289712, 289713) c6( ) c7(eW, T, cdv, Y, cz, mw, mt, nA, it, cd, cp); #c1(MED23) c2(XP_011534559) c3(mediator of RNA polymerase II transcription subunit 23 isoform X2) c4(331103) c5(331104) c6( ) c7(eW, T, cdv, Y, cz, mw, mt, nA, it, cd, cp); #c1(MED24) c2(NP_001072986) c3(mediator of RNA polymerase II transcription subunit 24 isoform 2) c4(35446) c5(35447, 35448, 35449, 35450, 35451, 35452, 35453, 35454, 35455, 35456, 35457, 35458, 35459, 35460) c6( ) c7(bM); #c1(MED24) c2(NP_055630) c3(mediator of RNA polymerase II transcription subunit 24 isoform 1) c4(182366, 182367, 182368) c5(182369, 182370, 182371, 182372, 182373, 182374, 182375, 182376, 182377, 182378, 182379, 182380, 182381, 182382, 182383, 182384, 182385, 182386, 182387, 182388, 182389, 182390, 182391, 182392, 182393, 182394, 182395, 182396, 182397, 182398, 182399, 182400) c6( ) c7(bM); #c1(MED24) c2(XP_005257931) c3(mediator of RNA polymerase II transcription subunit 24 isoform X10) c4(275424, 275425, 275426) c5(275427, 275428, 275429, 275430, 275431, 275432, 275433) c6( ) c7(bM); #c1(MED24) c2(XP_011523831) c3(mediator of RNA polymerase II transcription subunit 24 isoform X1) c4( ) c5(318407) c6( ) c7(bM); #c1(MED24) c2(XP_011523833) c3(mediator of RNA polymerase II transcription subunit 24 isoform X4) c4(318408) c5( ) c6( ) c7(bM); #c1(MED24) c2(XP_011523837) c3(mediator of RNA polymerase II transcription subunit 24 isoform X7) c4(318409) c5( ) c6( ) c7(bM); #c1(MED24) c2(XP_011523838) c3(mediator of RNA polymerase II transcription subunit 24 isoform X9) c4(318410, 318411) c5( ) c6( ) c7(bM); #c1(MED24) c2(XP_011523839) c3(mediator of RNA polymerase II transcription subunit 24 isoform XII) c4( ) c5(318412, 318413, 318414, 318415, 318416, 318417, 318418, 318419, 318420, 318421, 318422, 318423, 318424, 318425, 318426) c6( ) c7(bM); #c1(MED24) c2(XP_011523840) c3(mediator of RNA polymerase II transcription subunit 24 isoform X12) c4( ) c5(318427, 318428, 318429, 318430, 318431, 318432) c6( ) c7(bM); #c1(MED25) c2(NP_112235) c3(mediator of RNA polymerase II transcription subunit 25) c4(220998, 220999, 221000, 221001) c5(221002, 221003, 221004, 221005, 221006, 221007, 221008, 221009, 221010, 221011, 221012, 221013, 221014, 221015, 221016, 221017, 221018, 221019, 221020, 221021, 221022, 221023, 221024, 221025, 221026, 221027, 221028, 221029, 221030, 221031, 221032, 221033, 221034, 221035, 221036, 221037, 221038, 221039, 221040, 221041, 221042, 221043, 221044, 221045, 221046, 221047, 221048, 221049, 221050, 221051, 221052, 221053, 221054, 221055, 221056) c6( ) c7(bAQ, OW, aTT, aW, cdw, aT, uu, fu, bX, bU, f, bNM, B); #c1(MED25) c2(XP_011525655) c3(mediator of RNA polymerase II transcription subunit 25 isoform X1) c4(320055, 320056, 320057, 320058, 320059, 320060) c5(320061, 320062, 320063, 320064, 320065, 320066) c6( ) c7(bAQ, OW, aTT, aW, cdw, aT, uu, fu, bX, bU, f, bNM, B); #c1(MED28) c2(NP_079481) c3(mediator of RNA polymerase II transcription subunit 28) c4(218277) c5(218278, 218279, 218280, 218281, 218282, 218283) c6( ) c7(aW, ae, ad, aT); #c1(MED29) c2(NP_060062) c3(mediator of RNA polymerase II transcription subunit 29) c4(193948, 193949) c5(193950, 193951, 193952, 193953, 193954, 193955, 193956, 193957, 193958, 193959, 193960, 193961) c6( ) c7(aE); #c1(MED30) c2(NP_542382) c3(mediator of RNA polymerase II transcription subunit 30 isoform 1) c4(232316) c5(232317, 232318, 232319, 232320, 232321, 232322) c6( ) c7(If); #c1(MED4) c2(NP_001257558) c3(mediator of RNA polymerase II transcription subunit 4 isoform 2) c4( ) c5(95505, 95506, 95507, 95508, 95509) c6( ) c7(dP, bP); #c1(MED4) c2(NP_054885) c3(mediator of RNA polymerase II transcription subunit 4 isoform 1) c4(178190, 178191, 178192, 178193) c5(178194, 178195, 178196, 178197, 178198, 178199, 178200, 178201, 178202, 178203, 178204, 178205, 178206, 178207, 178208, 178209, 178210, 178211, 178212) c6( ) c7(dP, bP); #c1(MED6) c2(NP_001271138) c3(mediator of RNA polymerase II transcription subunit 6 isoform 1) c4(103453) c5(103454, 103455, 103456, 103457, 103458, 103459) c6( ) c7(dY); #c1(MED6) c2(NP_001271139) c3(mediator of RNA polymerase II transcription subunit 6 isoform 3) c4(103460) c5(103461, 103462, 103463, 103464, 103465, 103466) c6( ) c7(dY); #c1(MED6) c2(NP_005457) c3(mediator of RNA polymerase II transcription subunit 6 isoform 2) c4(156339, 156340) c5(156341, 156342, 156343, 156344, 156345, 156346, 156347, 156348, 156349, 156350, 156351, 156352, 156353) c6( ) c7(dY); #c1(MED9) c2(NP_060489) c3(mediator of RNA polymerase Il transcription subunit 9) c4(196224) c5(196225, 196226, 196227, 196228, 196229, 196230) c6( ) c7(yu, bX); #c1(MEF2A) c2(NP_005578) c3(myocyte-specific enhancer factor 2A isoform 1) c4(157607) c5(157608, 157609, 157610, 157611, 157612, 157613, 157614) c6( ) c7(bL, aZ, oe, ha, e, aw, cz, aj, j, cd, aR, J, cc, cdx, aT, vc, K, bq, af, jf, kl, anR, lR, Qr, aW, bN, dR, wk, mx, gZ, nw, B); #c1(MEF2A) c2(XP_005254972) c3(myocyte-specific enhancer factor 2A isoform X3) c4(273430) c5(273431, 273432, 273433, 273434, 273435, 273436, 273437, 273438, 273439, 273440) c6( ) c7(bL, aZ, oe, ha, e, aw, cz, aj, j, cd, aR, J, cc, cdx, aT, vc, K, bq, af, jf, kl, anR, lR, Qr, aW, bN, dR, wk, mx, gZ, nw, B); #c1(MEF2A) c2(XP_005254973) c3(myocyte-specific enhancer factor 2A isoform X12) c4( ) c5(273441, 273442, 273443, 273444, 273445, 273446, 273447, 273448, 273449, 273450, 273451, 273452, 273453, 273454, 273455, 273456, 273457, 273458, 273459, 273460, 273461, 273462, 273463, 273464, 273465, 273466, 273467, 273468, 273469) c6( ) c7(bL, aZ, oe, ha, e, aw, cz, aj, j, cd, aR, J, cc, cdx, aT, vc, K, bq, af, jf, kl, anR, lR, Or, aW, bN, dR, wk, mx, gZ, nw, B); #c1(MEF2A) c2(XP_011519886) c3(myocyte-specific enhancer factor 2A isoform X6) c4(314650, 314651) c5(314652, 314653, 314654, 314655, 314656, 314657, 314658, 314659, 314660, 314661, 314662, 314663) c6( ) c7(bL, aZ, oe, ha, e, aw, cz, aj, j, cd, aR, J, cc, cdx, aT, vc, K, bq, af, jf, kl, anR, lR, Qr, aW, bN, dR, wk, mx, gZ, nw, B); #c1(MEF2A) c2(XP_011519888) c3(myocyte-specific enhancer factor 2A isoform X1) c4(314664) c5(314665, 314666, 314667, 314668, 314669, 314670) c6( ) c7(bL, aZ, oe, ha, e, aw, cz, aj, j, cd, aR, J, cc, cdx, aT, vc, K, bq, af, jf, kl, anR, lR, Or, aW, bN, dR, wk, mx, gZ, nw, B); #c1(MEF2A) c2(XP_011519889) c3(myocyte-specific enhancer factor 2A isoform X2) c4(314671) c5(314672, 314673, 314674, 314675, 314676, 314677) c6( ) c7(bL, aZ, oe, ha, e, aw, cz, aj, j, cd, aR, J, cc, cdx, aT, vc, K, bq, af, jf, kl, anR, lR, Or, aW, bN, dR, wk, mx, gZ, nw, B); #c1(MEF2A) c2(XP_011519892) c3(myocyte-specific enhancer factor 24 isoform XII) c4(314678, 314679) c5( ) c6( ) c7(bL, aZ, oe, ha, e, aw, cz, aj, j, cd, aR, J, cc, cdx, aT, vc, K, bq, af, jf, kl, anR, lR, Ar, aW, bN, dR, wk, mx, gZ, nw, B); #c1(MEF2B) c2(NP_001139257) c3(myocyte-specific enhancer factor 2B) c4(59016, 59017) c5(59018, 59019, 59020, 59021, 59022, 59023, 59024, 59025, 59026, 59027, 59028, 59029, 59030, 59031, 59032, 59033, 59034, 59035) c6( ) c7(V); #c1(MEF2BNB-MEF2B) c2(NP_005910) c3(myocyte enhancer factor 28 isoform b) c4(160312) c5(160313, 160314, 160315, 160316, 160317, 160318, 160319) c6( ) c7(V); #c1(MEF2C) c2(NP_001124477) c3(myocyte-specific enhancer factor 2C isoform 2) c4(49122, 49123, 49124) c5(49125, 49126, 49127, 49128, 49129, 49130, 49131) c6( ) c7(bL, gh, cdy, oe, ha, e, aw, amj, bS, nA, cd, aR, cc, aEX, nX, aT, h, K, bq, aqa, alW, VX, lR, eZ, f, bN, ck, iT, wK, agl, nw, WI, bt, cB, B); #c1(MEF2C) c2(NP_001180276) c3(myocyte-specific enhancer factor 2C isoform 3) c4(73674) c5(73675, 73676, 73677, 73678, 73679, 73680, 73681) c6( ) c7(bL, gh, cdy, oe, ha, e, aw, amj, bS, nA, cd, aR, cc, aEX, nX, aT, h, K, bq, aqa, alW, VX, lR, eZ, f, bN, ck, iT, wK, agl, nw, Wl, bt, cB, B); #c1(MEF2C) c2(NP_001180277) c3(myocyte-specific enhancer factor 2C isoform 4) c4(73682) c5(73683, 73684, 73685, 73686, 73687, 73688, 73689, 73690) c6( ) c7(bL, gh, cdy, oe, ha, e, aw, amj, bS, nA, cd, aR, cc, aEX, nX, aT, h, K, bq, aqa, alW, VX, lR, eZ, f, bN, ck, iT, wK, agl, nw, Wl, bt, cB, B); #c1(MEF2C) c2(NP_001180278) c3(myocyte-specific enhancer factor 2C isoform 5) c4( ) c5(73691) c6( ) c7(bL, gh, cdy, De, ha, e, aw, amj, bS, nA, cd, aR, cc, aEX, nX, aT, h, K, bq, aqa, alW, VX, lR, eZ, f, bN, ck, iT, wK, agl, nw, Wl, bt, cB, B); #c1(MEF2C) c2(NP_001180279) c3(myocyte-specific enhancer factor 2C isoform 1) c4(73692, 73693, 73694, 73695) c5(73696, 73697, 73698, 73699, 73700, 73701, 73702, 73703, 73704, 73705, 73706, 73707, 73708, 73709, 73710, 73711, 73712, 73713, 73714) c6( ) c7(bL, gh, cdy, De, ha, e, aw, amj, bS, nA, cd, aR, cc, aEX, nX, aT, h, K, bq, aqa, alW, VX, lR, eZ, f, bN, ck, iT, wK, agl, nw, Wl, bt, cB, B); #c1(MEF2C) c2(XP_011541699) c3(myocyte-specific enhancer factor 2C isoform X6) c4(340221, 340222) c5( ) c6( ) c7(bL, gh, cdy, oe, ha, e, aw, amj, bS, nA, cd, aR, cc, aEX, nX, aT, h, K, bq, aqa, alW, VX, lR, eZ, f, bN, ck, iT, wK, agl, nw, Wl, bt, cB, B); #c1(MEF2C) c2(XP_011541702) c3(myocyte-specific enhancer factor 2C isoform X8) c4(340223) c5(340224, 340225, 340226, 340227, 340228, 340229) c6( ) c7(bL, gh, cdy, De, ha, e, aw, amj, bS, nA, cd, aR, cc, aEX, nX, aT, h, K, bq, aqa, alW, VX, lR, eZ, f, bN, ck, iT, wK, agl, nw, Wl, bt, cB, B); #c1(MEF2C) c2(XP_011541703) c3(myocyte-specific enhancer factor 2C isoform X9) c4(340230, 340231) c5(340232, 340233, 340234, 340235, 340236, 340237, 340238, 340239, 340240, 340241, 340242, 340243, 340244, 340245, 340246, 340247, 340248, 340249) c6( ) c7(bL, gh, cdy, De, ha, e, aw, amj, bS, nA, cd, aR, cc, aEX, nX, aT, h, K, bq, aqa, alW, VX, lR, eZ, f, bN, ck, iT, wK, agl, nw, Wl, bt, cB, B); #c1(MEF2C) c2(XP_011541704) c3(myocyte-specific enhancer factor 2C isoform X10) c4(340250) c5(340251, 340252, 340253, 340254, 340255, 340256, 340257, 340258, 340259, 340260, 340261, 340262, 340263, 340264, 340265, 340266, 340267, 340268) c6( ) c7(bL, gh, cdy, De, ha, e, aw, amj, bS, nA, cd, aR, cc, aEX, nX, aT, h, K, bq, aqa, alW, VX, lR, eZ, f, bN, ck, iT, wK, agl, nw, Wl, bt, cB, B); #c1(MEF2D) c2(XP_005245226) c3(myocyte-specific enhancer factor 20 isoform X1) c4(266357) c5(266358, 266359, 266360, 266361, 266362, 266363, 266364) c6( ) c7(bL, ck, T, bv, fM, ac, x, an, aj, ur, K, k, uu, vo, B); #c1(MEF2D) c2(XP_006711395) c3(myocyte-specific enhancer factor 2D isoform X3) c4(285972, 285973) c5(285974, 285975, 285976, 285977, 285978, 285979, 285980, 285981, 285982, 285983, 285984, 285985, 285986) c6( ) c7(bL, ck, T, bv, fM, ac, x, an, aj, ur, K, k, uu, vo, B); #c1(MEF2D) c2(XP_006711396) c3(myocyte-specific enhancer factor 20 isoform X2) c4(285987, 285988) c5(285989, 285990, 285991, 285992, 285993, 285994, 285995, 285996, 285997, 285998) c6( ) c7(bL, ck, T, bv, fM, ac, x, an, aj, ur, K, k, uu, vo, B); #c1(MEF2D) c2(XP_006711397) c3(myocyte-specific enhancer factor 20 isoform X4) c4(285999) c5(286000, 286001, 286002, 286003, 286004, 286005, 286006) c6( ) c7(bL, ck, T, bv, fM, ac, x, an, aj, ur, K, k, uu, vo, B); #c1(MEF2D) c2(XP_011507872) c3(myocyte-specific enhancer factor 2D isoform X5) c4( ) c5(298069, 298070, 298071, 298072, 298073, 298074) c6( ) c7(bL, ck, T, bv, fM, ac, x, an, aj, ur, K, k, uu, vo, B); #c1(MEFV) c2(NP_000234) c3(pyrin isoform 1) c4(5285, 5286, 5287, 5288) c5(5289, 5290, 5291, 5292, 5293, 5294, 5295, 5296, 5297, 5298, 5299, 5300, 5301, 5302, 5303, 5304, 5305, 5306, 5307, 5308, 5309, 5310, 5311, 5312, 5313, 5314) c6( ) c7(bM, T, ea, fd, cl, cdA, yM, Vp, K, aWf, hH, hm, eG, cdz, aW, asD, ih, bw, iT, qr, m, R, dD, gU, aw, pn, aPb, ai, br, bKk, rC, h, By, wb, ey, NX, mR, qY, lb, fe, pm, bQL, Zb, cM, hs, B, bL, eR, j, C, V, eA, o, cF, cdC, nX, ld, aT, DX, af, iU, cdB, aLJ, t, iK, dS, hc, hO, wR, Da, fD, bt, ao, qX, CS, dM, jh, cdD, eq, ad, wD, fU, NY, ji, sj, d, eZ, anW, iV, YZ, agn, dr, aRk, sG, Lz, y, ra, hv); #c1(MEFV) c2(NP_001185465) c3(pyrin isoform 2) c4(75654, 75655, 75656) c5(75657, 75658, 75659, 75660, 75661, 75662, 75663, 75664, 75665, 75666, 75667, 75668, 75669, 75670, 75671, 75672, 75673) c6( ) c7(bM, T, ea, fd, cl, cdA, yM, Vp, K, aWf, hH, hm, eG, cdz, aW, asD, ih, bw, iT, qr, m, R, dD, gU, aw, pn, aPb, ai, br, bKk, rC, h, By, wb, ey, NX, mR, qY, lb, fe, pm, bQL, Zb, cM, hs, B, bL, eR, j, C, V, eA, o, cF, cdC, nX, ld, aT, DX, af, iU, cdB, aLJ, t, iK, dS, hc, hO, wR, Da, fD, bt, ao, qX, CS, dM, jh, cdD, eq, ad, wD, fU, NY, ji, sj, d, eZ, anW, iV, YZ, agn, dr, aRk, sG, Lz, y, ra, hv); #c1(MEGF10) c2(NP_115822) c3(multiple epidermal growth factor-like domains protein 10 precursor) c4(224122, 224123) c5(224124, 224125, 224126, 224127, 224128, 224129, 224130, 224131, 224132, 224133) c6( ) c7(dP, cdE, oj, Ad, pN); #c1(MEGF11) c2(NP_115821) c3(multiple epidermal growth factor-like domains protein fl precursor) c4(224091, 224092, 224093) c5(224094, 224095, 224096, 224097, 224098, 224099, 224100, 224101, 224102, 224103, 224104, 224105, 224106, 224107, 224108, 224109, 224110, 224111, 224112, 224113, 224114, 224115, 224116, 224117, 224118, 224119, 224120, 224121) c6( ) c7(aw, fq, j, ac, Ea); #c1(MEGF11) c2(XP_011520411) c3(multiple epidermal growth factor-like domains protein Il isoform X1) c4(315292) c5(315293, 315294, 315295, 315296, 315297) c6( ) c7(aw, fq, j, ac, Ea); #c1(MEGF8) c2(NP_001258867) c3(multiple epidermal growth factor-like domains protein 8 isoform 1 precursor) c4(96612) c5(96613, 96614, 96615, 96616, 96617, 96618, 96619) c6( ) c7(T, pf, bE, bDi, aW, cdF, axc, OS, n, aT, bDl, r, K, q, aA, bB, bD, g, aF, B); #c1(MEGF8) c2(NP_001401) c3(multiple epidermal growth factor-like domains protein 8 isoform 2 precursor) c4(117593, 117594) c5(117595, 117596, 117597, 117598, 117599, 117600, 117601, 117602, 117603, 117604, 117605, 117606, 117607) c6( ) c7(T, pf, bE, bDi, aW, cdF, axc, OS, n, aT, bDl, r, K, q, aA, bB, bD, g, aF, B); #c1(MEGF9) c2(NP_001073966) c3(multiple epidermal growth factor-like domains protein 9 precursor) c4(36734, 36735) c5(36736, 36737, 36738, 36739, 36740, 36741, 36742, 36743, 36744, 36745, 36746, 36747) c6( ) c7(aW, aT); #c1(MEI1) c2(NP_689726) c3(meiosis inhibitor protein 1) c4(243679, 243680) c5(243681, 243682, 243683, 243684, 243685, 243686) c6( ) c7(vH); #c1(MEI1) c2(XP_011528242) c3(meiosis inhibitor protein 1 isoform X6) c4(323308) c5(323309, 323310, 323311, 323312, 323313, 323314, 323315) c6( ) c7(vH); #c1(MEI1) c2(XP_011528243) c3(meiosis inhibitor protein 1 isoform X7) c4(323316) c5(323317, 323318, 323319, 323320, 323321, 323322) c6( ) c7(vH); #c1(MEI1) c2(XP_011528253) c3(meiosis inhibitor protein 1 isoform X17) c4(323323) c5(323324, 323325, 323326, 323327, 323328) c6( ) c7(vH); #c1(MEI1) c2(XP_011528256) c3(meiosis inhibitor protein 1 isoform X20) c4( ) c5(323329, 323330, 323331, 323332, 323333, 323334) c6( ) c7(vH); #c1(MEI1) c2(XP_011528257) c3(meiosis inhibitor protein 1 isoform X21) c4(323335) c5(323336, 323337, 323338, 323339, 323340, 323341) c6( ) c7(vH); #c1(MEIS1) c2(NP_002389) c3(homeobox protein Meis1) c4(128053) c5(128054, 128055, 128056, 128057, 128058, 128059, 128060, 128061, 128062, 128063, 128064, 128065, 128066, 128067, 128068, 128069, 128070) c6( ) c7(T, eR, yY, EQ, ci, hF, aY, q, br, ad, x, hb, aT, dz, jj, ae, aW, yL, r, cm, bw, fq, ck, iT, k, bD, bt, kS, X, awm); #c1(MEIS1) c2(XP_005264378) c3(homeobox protein Meis1 isoform X1) c4(278836) c5( ) c6( ) c7(T, eR, yY, EQ, ci, hF, aY, q, br, ad, x, hb, aT, dz, jj, ae, aW, yL, r, cm, bw, fq, ck, iT, k, bD, bt, kS, X, awm); #c1(MEIS1) c2(XP_005264379) c3(homeobox protein Meis1 isoform X2) c4(278837, 278838) c5(278839, 278840, 278841, 278842, 278843, 278844, 278845, 278846, 278847, 278848, 278849, 278850, 278851, 278852) c6( ) c7(T, eR, yY, EQ, ci, hF, aY, q, br, ad, x, hb, aT, dz, jj, ae, aW, yL, r, cm, bw, fq, ck, iT, k, bD, bt, KS, X, awm); #c1(MEIS1) c2(XP_005264380) c3(homeobox protein Meis1 isoform X3) c4(278853, 278854) c5( ) c6( ) c7(T, eR, yY, EQ, ci, hF, aY, q, br, ad, x, hb, aT, dz, jj, ae, aW, yL, r, cm, bw, fq, ck, iT, k, bD, bt, KS, X, awm); #c1(MEIS1) c2(XP_005264381) c3(homeobox protein Meis1 isoform X4) c4( ) c5(278855, 278856) c6( ) c7(T, eR, yY, EQ, ci, hF, aY, q, br, ad, x, hb, aT, dz, jj, ae, aW, yL, r, cm, bw, fq, ck, iT, k, bD, bt, kS, X, awm); #c1(MEIS1) c2(XP_005264382) c3(homeobox protein Meis1 isoform X5) c4(278857, 278858, 278859) c5(278860, 278861, 278862, 278863, 278864, 278865, 278866, 278867, 278868, 278869, 278870, 278871, 278872, 278873, 278874, 278875, 278876, 278877, 278878, 278879, 278880, 278881) c6( )c7(T, eR, yY, EQ, ci, hF, aY, q, br, ad, x, hb, aT, dz, j, ae, aW, yL, r, cm, bw, fq, ck, iT, k, bD, bt, KS, X, awm); #c1(MEIS2) c2(NP_002390) c3(homeobox protein Meis2 isoform f) c4(128071, 128072) c5(128073, 128074, 128075, 128076, 128077, 128078, 128079, 128080, 128081, 128082) c6( )c7(e, fq, rE, cm, ae, ad, qc); #c1(MEIS2) c2(NP_733774) c3(homeobox protein Meis2 isoform b) c4(247026) c5(247027, 247028, 247029, 247030, 247031, 247032, 247033, 247034, 247035) c6( )c7(e, fq, rE, cm, ae, ad, qc); #c1(MEIS2) c2(NP_733776) c3(homeobox protein Meis2 isoform d) c4(247036) c5(247037, 247038, 247039, 247040, 247041, 247042, 247043, 247044, 247045, 247046, 247047, 247048, 247049, 247050, 247051) c6( )c7(e, fq, rE, cm, ae, ad, qc); #c1(MEIS2) c2(NP_733777) c3(homeobox protein Meis2 isoform a) c4(247052, 247053) c5(247054, 247055, 247056, 247057, 247058) c6( )c7(e, fq, rE, cm, ae, ad, qc); #c1(MEIS2) c2(NP_758526) c3(homeobox protein Meis2 isoform g) c4( )c5(249253, 249254, 249255, 249256, 249257) c6( )c7(e, fq, rE, cm, ae, ad, qc); #c1(MEIS2) c2(NP_758527) c3(homeobox protein Meis2 isoform h) c4(249258) c5(249259, 249260, 249261, 249262, 249263, 249264, 249265, 249266, 249267, 249268, 249269, 249270, 249271) c6( )c7(e, fq, rE, cm, ae, ad, qc); #c1(MEIS2) c2(XP_006720585) c3(homeobox protein Meis2 isoform XI) c4(293558) c5(293559, 293560, 293561, 293562, 293563, 293564, 293565, 293566, 293567, 293568, 293569, 293570) c6( )c7(e, fq, rE, cm, ae, ad, qc); #c1(MEIS2) c2(XP_006720588) c3(homeobox protein Meis2 isoform X4) c4(293571) c5(293572) c6( )c7(e, fq, rE, cm, ae, ad, qc); #c1(MEIS2) c2(XP_006720589) c3(homeobox protein Meis2 isoform X5) c4( )c5(293573) c6( )c7(e, fq, rE, cm, ae, ad, qc); #c1(MEIS2) c2(XP_006720592) c3(homeobox protein Meis2 isoform X7) c4(293574) c5(293575, 293576, 293577, 293578, 293579) c6( )c7(e, fq, rE, cm, ae, ad, qc); #c1(MELK) c2(NP_001243614) c3(maternal embryonic leucine zipper kinase isoform 2) c4(91206) c5(91207, 91208, 91209, 91210, 91211, 91212) c6( )c7(T, d, dB, aW, qy, aT, aA, KO, aF, ey, K, as, eA, ae, B, ad); #c1(MELK) c2(NP_001243616) c3(maternal embryonic leucine zipper kinase isoform 3) c4(91213, 91214) c5(91215, 91216, 91217, 91218, 91219, 91220) c6( )c7(T, d, dB, aW, qy, aT, aA, KO, aF, ey, K, as, eA, a, B, ad); #c1(MELK) c2(NP_001243617) c3(maternal embryonic leucine zipper kinase isoform 4) c4(91221) c5(91222, 91223, 91224, 91225, 91226, 91227) c6( )c7(T, d, dB, aW, qy, aT, aA, KO, aF, ey, K, as, eA, ae, B, ad); #c1(MELK) c2(NP_001243619) c3(maternal embryonic leucine zipper kinase isoform 6) c4(91228) c5(91229, 91230, 91231, 91232, 91233, 91234) c6( )c7(T, d, dB, aW, qy, aT, aA, KO, aF, ey, K, as, eA, a, B, ad); #c1(MELK) c2(NP_001243620) c3(maternal embryonic leucine zipper kinase isoform 7) c4( )c5(91235) c6( )c7(T, d, dB, aW, qy, aT, aA, KO, aF, ey, K, as, eA, ae, B, ad); #c1(MELK) c2(NP_001243621) c3(maternal embryonic leucine zipper kinase isoform 8) c4( )c5(91236, 91237, 91238, 91239, 91240) c6( )c7(T, d, dB, aW, qy, aT, aA, KO, aF, ey, K, as, eA, ae, B, ad); #c1(MELK) c2(NP_055606) c3(maternal embryonic leucine zipper kinase isoform 1) c4(182206, 182207) c5(182208, 182209, 182210, 182211, 182212, 182213, 182214, 182215, 182216, 182217, 182218) c6( )c7(T, d, dB, aW, qy, aT, aA, KO, aF, ey, K, as, eA, ae, B, ad); #c1(MELK) c2(XP_011516386) c3(maternal embryonic leucine zipper kinase isoform X1) c4(309611) c5(309612, 309613, 309614, 309615, 309616, 309617, 309618) c6( )c7(T, d, dB, aW, qy, aT, aA, KO, aF, ey, K, as, eA, a, B, ad); #c1(MEMO1) c2(NP_001131074) c3(protein MEMO1 isoform 2) c4(52571, 52572) c5(52573, 52574, 52575, 52576, 52577, 52578) c6( )c7(cm); #c1(MEMO1) c2(NP_001288762) c3(protein MEMO1 isoform 1) c4(113994, 113995) c5(113996, 113997, 113998, 113999, 114000, 114001, 114002, 114003, 114004, 114005, 114006, 114007, 114008, 114009, 114010, 114011) c6( )c7(cm); #c1(MEMO1) c2(XP_011531199) c3(protein MEMO1 isoform X4) c4( )c5(327153, 327154, 327155, 327156, 327157, 327158) c6( )c7(cm); #c1(MEN1) c2(NP_570711) c3(menin isoform 2) c4(234146) c5(234147, 234148, 234149, 234150, 234151, 234152, 234153, 234154, 234155, 234156, 234157, 234158, 234159, 234160, 234161, 234162, 234163, 234164, 234165, 234166, 234167, 234168, 234169, 234170, 234171, 234172, 234173, 234174, 234175, 234176, 234177, 234178, 234179, 234180, 234181, 234182, 234183, 234184, 234185, 234186, 234187, 234188, 234189, 234190, 234191, 234192, 234193, 234194, 234195, 234196, 234197, 234198, 234199, 234200) c6( ) c7(T, aC, Fj, vf, if, e, abD, ci, aY, ip, AW, aE, pX, vg, iv, K, eL, azh, li, yo, avH, aW, kt, aB, g, eF, cm, aoS, qJ, eM, nB, TG, vA, iS, beI, QF, azg, OD, iu, lT, n, aj, aKt, OA, ml, qN, JW, zw, Do, KN, KH, jy, h, dL, Xh, Oz, aAH, aeu, ahJ, Hu, hq, bzw, aKA, YE, hk, bo, hj, HR, bUG, HB, anx, B, bL, eR, boq, fZ, cz, wo, j, q, J, eJ, UE, Zp, mj, nv, cdG, aT, aHs, blg, ae, jf, Uf, bID, s, Y, aNi, hf, t, ck, nM, iM, nG, hr, aJQ, VB, aAO, bNx, hl, pY, Oy, aD, jh, OE, KO, hF, ad, iC, av, bkq, Rq, yh, bYY, Yi, aAL, je, vj, eH, hp, HI, bz, aTb, dr, bF, Ew, AR, cdH, X); #c1(MEN1) c2(NP_570713) c3(menin isoform 1) c4(234201) c5(234202, 234203, 234204, 234205, 234206, 234207, 234208, 234209, 234210, 234211, 234212, 234213, 234214, 234215, 234216, 234217, 234218, 234219, 234220, 234221, 234222, 234223) c6( ) c7(T, aC, Fj, vf, if, e, abD, ci, aY, ip, AW, aE, pX, vg, iv, K, eL, azh, li, yo, avH, aW, kt, aB, g, eF, cm, aoS, qJ, eM, nB, TG, vA, iS, beI, QF, azg, OD, iu, lT, n, aj, aKt, OA, ml, qN, JW, zw, Do, KN, KH, jy, h, dL, Xh, Oz, aAH, aeU, ahJ, Hu, hq, bzw, aKA, YE, hk, bo, hj, hR, bUG, HB, anx, B, bL, eR, boq, fZ, cz, wo, j, q, J, eJ, UE, Zp, mj, nv, cdG, aT, aHs, blg, ae, jf, Uf, bID, s, Y, aNi, hf, t, ck, nM, iM, nG, hr, aJQ, VB, aAO, bNx, hl, pY, Oy, aD, jh, OE, KO, hF, ad, iC, av, bkq, Rq, yh, bYY, Yi, aAL, je, vj, eH, hp, HI, bz, aTb, dr, bF, Ew, AR, cdH, X); #c1(MEN1) c2(XP_005274058) c3(menin isoform X1) c4(284912, 284913, 284914, 284915, 284916, 284917) c5( ) c6( ) c7(T, aC, Fj, vf, if, e, abD, ci, aY, ip, AW, aE, pX, vg, iv, K, eL, azh, li, yo, avH, aW, kt, aB, g, eF, cm, aoS, qJ, eM, nB, TG, vA, iS, beI, QF, azg, OD, iu, lT, n, aj, aKt, OA, ml, qN, JW, zw, Do, KN, KH, jy, h, dL, Xh, Oz, aAH, aeU, ahJ, Hu, hq, bzw, aKA, YE, hk, bo, hj, hR, bUG, HB, anx, B, bL, eR, boq, fZ, cz, wo, j, q, J, eJ, UE, Zp, mj, nv, cdG, aT, aHs, blg, ae, jf, Uf, bID, s, Y, aNi, hf, t, ck, nM, iM, nG, hr, aJQ, VB, aAO, bNx, hl, pY, Oy, aD, jh, OE, KO, hF, ad, iC, av, bkq, Rq, yh, bYY, Yi, aAL, je, vj, eH, hp, HI, bz, aTb, dr, bF, Ew, AR, cdH, X); #c1(MEOX1) c2(NP_001035091) c3(homeobox protein MOX-1 isoform 3) c4( ) c5(32491, 32492, 32493, 32494, 32495) c6( ) c7(t, bAV, cdl, m, fJ); #c1(MEOX1) c2(NP_004518) c3(homeobox protein MOX-1 isoform 1) c4(147157, 147158) c5(147159, 147160, 147161, 147162, 147163, 147164, 147165, 147166) c6( ) c7(t, bAV, cdl, m, fJ); #c1(MEOX1) c2(NP_054705) c3(homeobox protein MOX-1 isoform 2) c4(177321) c5(177322, 177323, 177324, 177325, 177326, 177327) c6( ) c7(t, bAV, cdl, m, fJ); #c1(MEOX2) c2(NP_005915) c3(homeobox protein MOX-2) c4(160356) c5(160357, 160358, 160359, 160360, 160361, 160362, 160363) c6( ) c7(B, j, af); #c1(MEP1A) c2(NP_005579) c3(meprin A subunit alpha precursor) c4(157615) c5(157616, 157617, 157618, 157619, 157620, 157621) c6( ) c7(fe, uu, X, iV); #c1(MEPE) c2(NP_001171623) c3(matrix extracellular phosphoglycoprotein isoform a precursor) c4(70989, 70990, 70991) c5(70992, 70993, 70994, 70995, 70996, 70997, 70998, 70999, 71000, 71001, 71002, 71003, 71004, 71005, 71006, 71007) c6( ) c7(bfh, lD, T, ea, if, e, al, bO, bLf, bB, cL, ci, aY, x, Gt, aE, awe, wM, Ag, iI, iX, M, iv, gs, azm, Bi, bU, eL, hm, cdN, xQ, aW, hI, CT, YG, bxx, Ku, g, hJ, as, eF, cm, bw, ajj, bfw, eM, nB, dc, vA, mx, aep, cj, bXz, wx, k, m, NN, abh, dD, OI, aw, NT, Bm, n, aj, fr, nA, qN, cd, br, rc, afR, BI, ft, amB, cc, h, hn, dL, ux, ais, Lg, pI, beo, mR, A, aq, kp, Hu, YF, zZ, acD, op, lx, lJ, ky, dB, kz, hL, pg, oh, azr, ER, anx, it, B, bL, AB, AS, eR, jx, rf, aA, cz, cdP, j, aDO, wy, AO, V, biO, q, J, o, er, aOY, eA, mj, bIC, hW, cdJ, ld, aWJ, aT, dz, bK, j, ae, af, bA, jf, qw, arI, s, iI, xR, Y, afS, aeS, r, An, en, cl, t, iK, bN, avz, ck, dS, pH, BU, tY, kP, ac, rT, rR, iM, au, of, cdK, np, rK, fD, UF, cdM, gT, bt, ij, hS, hl, kh, id, qb, cdO, uO, bs, jN, jh, eq, fu, hF, cdL, aGW, ad, BY, Jm, hX, afb, hd, F, afm, vJ, bfZ, hD, bsR, auE, fb, vO, iL, d, azS, BO, je, cH, qt, ff, xs, eH, eZ, afi, Ej, gE, hp, aF, Fq, anW, bfH, bz, qa, Hp, axI, es, gk, eU, Ew, qe, pc, bF, bE, xT, ag, aS, bC, hE, X, y); #c1(MEPE) c2(NP_001171626) c3(matrix extracellular phosphoglycoprotein isoform b) c4( ) c5(71008, 71009, 71010, 71011, 71012, 71013, 71014, 71015, 71016) c6( ) c7(bfh, ID, T, ea, if, e, al, bO, bLf, bB, cL, ci, aY, x, Gt, aE, awe, wM, Ag, iI, iX, M, iv, gs, azm, Bi, bU, eL, hm, cdN, xQ, aW, hl, CT, YG, bxx, Ku, g, hJ, as, eF, cm, bw, aj, bfw, eM, nB, dc, vA, mx, aep, cj, bXz, wx, k, m, NN, abh, dD, OI, aw, NT, Bm, n, aj, fr, nA, qN, cd, br, rc, afR, BI, ft, amB, cc, h, hn, dL, ux, ais, Lg, pI, beo, mR, A, aq, kp, Hu, YF, zZ, acD, op, lx, lJ, ky, dB, kz, hL, pg, oh, azr, ER, anx, it, B, bL, AB, AS, eR, jx, rf, aA, cz, cdP, j, aDO, wy, AO, V, biO, q, J, o, er, aOY, eA, mj, bIC, hW, cdJ, ld, aWJ, aT, dz, bK, j, ae, af, bA, jf, qw, arI, s, iU, xR, Y, afS, aeS, r, An, en, cl, t, iK, bN, avz, ck, dS, pH, BU, tY, kP, ac, rT, rR, iM, au, of, cdk, np, rK, fD, UF, cdM, gT, bt, ij, hS, hl, kh, id, qb, cdO, uO, bs, jN, jh, eq, fu, hF, cdL, aGW, ad, BY, Jm, hX, afb, hd, F, afm, vJ, bfZ, hD, bsR, auE, fb, vO, iL, d, azS, BO, je, cH, qt, ff, xs, eH, eZ, afi, Ej, gE, hp, aF, Fq, anW, bfH, bz, qa, Hp, axI, es, gk, eU, Ew, qe, pc, bF, bE, xT, ag, aS, bC, hE, X, y); #c1(MEPE) c2(NP_001278112) c3(matrix extracellular phosphoglycoprotein isoform c precursor) c4( ) c5(109482, 109483) c6( ) c7(bfh, lD, T, ea, if, e, al, bO, bLf, bB, cL, ci, aY, x, Gt, aE, awe, wM, Ag, iI, iX, M, iv, gs, azm, Bi, bU, eL, hm, cdN, xQ, aW, hl, CT, YG, bxx, Ku, g, hJ, as, eF, cm, bw, ajj, bfw, eM, nB, dc, vA, mx, aep, cj, bXz, wx, k, m, NN, abh, dD, OI, aw, NT, Bm, n, aj, fr, nA, qN, cd, br, rc, afR, BI, ft, amB, cc, h, hn, dL, ux, ais, Lg, pI, beo, mR, A, aq, kp, Hu, YF, zZ, acD, op, lx, lJ, ky, dB, kz, hL, pg, oh, azr, ER, anx, it, B, bL, AB, AS, eR, jx, rf, aA, cz, cdP, j, aDO, wy, AO, V, biO, q, J, o, er, aOY, eA, mj, bIC, hW, cdJ, ld, aWJ, aT, dz, bK, jj, ae, af, bA, jf, qw, arI, s, iU, xR, Y, afS, aeS, r, An, en, cl, t, iK, bN, avz, ck, dS, pH, BU, tY, kP, ac, rT, rR, iM, au, of, cdk, np, rK, fD, UF, cdM, gT, bt, ij, hS, hl, kh, id, qb, cdO, uO, bs, jN, jh, eq, fu, hF, cdL, aGW, ad, BY, Jm, hX, afb, hd, F, afm, vJ, bfZ, hD, bsR, auE, fb, vO, iL, d, azS, BO, je, cH, qt, ff, xs, eH, eZ, afi, Ej, gE, hp, aF, Fq, anW, bfH, bz, qa, Hp, axI, es, gk, eU, Ew, qe, pc, bF, bE, xT, ag, aS, bC, hE, X, y); #c1(MERTK) c2(NP_006334) c3(tyrosine-protein kinase Mer precursor) c4(164102, 164103) c5(164104, 164105, 164106, 164107, 164108, 164109, 164110, 164111, 164112, 164113, 164114, 164115, 164116, 164117, 164118, 164119, 164120) c6( ) c7(T, dD, ea, nx, jh, oY, eq, ci, bX, V, mC, Nh, x, bv, cdQ, aT, hn, ey, d, lR, hG, aW, CM, wc, eZ, nK, jT, nC, ck, dB, nw, cS, k, np, fL, wx, bt); #c1(MERTK) c2(XP_011508792) c3(tyrosine-protein kinase Mer isoform X2) c4(299388) c5( ) c6( ) c7(T, dD, ea, nx, jh, oY, eq, ci, bX, V, mC, Nh, x, bv, cdQ, aT, hn, ey, d, IR, hG, aW, CM, wc, eZ, nK, jT, nC, ck, dB, nw, cS, k, np, fL, wx, bt); #c1(MERTK) c2(XP_011508793) c3(tyrosine-protein kinase Mer isoform X4) c4( ) c5(299389, 299390, 299391, 299392, 299393) c6( ) c7(T, dD, ea, nx, jh, oY, eq, ci, bX, V, mC, Nh, x, bv, cdQ, aT, hn, ey, d, lR, hG, aW, CM, wc, eZ, nK, jT, nC, ck, dB, nw, cS, k, np, fL, wx, bt); #c1(MESDC1) c2(NP_072088) c3(mesoderm development candidate 1) c4(212645) c5(212646, 212647, 212648, 212649, 212650, 212651) c6( ) c7(eL, oe, aw, dL); #c1(MESDC2) c2(NP_055969) c3(LDLR chaperone MESO precursor) c4(184789) c5(184790, 184791, 184792, 184793, 184794, 184795, 184796, 184797, 184798, 184799) c6( ) c7(rO); #c1(MESP2) c2(NP_001035047) c3(mesoderm posterior protein 2) c4(32475) c5(32476, 32477, 32478, 32479, 32480, 32481, 32482, 32483) c6( ) c7(cdR, bfU, bga); #c1(MEST) c2(NP_001240831) c3(mesoderm-specific transcript homolog protein isoform d precursor) c4(88881) c5(88882, 88883, 88884, 88885, 88886, 88887, 88888, 88889, 88890, 88891, 88892, 88893, 88894, 88895, 88896, 88897, 88898, 88899, 88900, 88901, 88902, 88903, 88904, 88905, 88906, 88907, 88908, 88909, 88910, 88911, 88912, 88913, 88914, 88915, 88916, 88917, 88918, 88919, 88920, 88921, 88922, 88923) c6( ) c7(iu, T, gU, ND, bE, aW, LO, ml, mg, aT, bB, at, dL, v, ci, m, qM, g, t, bp, B); #c1(MEST) c2(NP_002393) c3(mesoderm-specific transcript homolog protein isoform a) c4(128090) c5(128091) c6( ) c7(iu, T, gU, ND, bE, aW, LO, ml, mg, aT, bB, at, dL, v, ci, m, qM, g, t, bp, B); #c1(MEST) c2(NP_803491) c3 (mesoderm-specific transcript homolog protein isoform b precursor) c4(252636) c5(252637, 252638, 252639, 252640, 252641, 252642, 252643, 252644, 252645, 252646, 252647, 252648, 252649, 252650, 252651, 252652, 252653, 252654, 252655, 252656, 252657) c6( ) c7(iu, T, gU, ND, bE, aW, LO, ml, mg, aT, bB, at, dL, v, ci, m, qM, g, t, bp, B); #c1(MEST) c2(XP_011514524) c3(mesoderm-specific transcript homolog protein isoform X1) c4(307214, 307215, 307216, 307217, 307218) c5(307219, 307220, 307221, 307222) c6( ) c7(iu, T, gU, ND, bE, aW, LO, ml, mg, aT, bB, at, dL, v, ci, m, qM, g, t, bp, B); #c1(MET) c2(NP_000236) c3(hepatocyte growth factor receptor isoform b precursor) c4(5315) c5(5316, 5317, 5318, 5319, 5320) c6( ) c7(T, aC, aCh, pf, bll, al, bO, fd, bB, ci, aY, sD, lS, bv, aE, Xc, pX, Qk, ja, Vv, Ou, HR, K, hu, pk, eL, KC, eG, iE, Jl, aW, fc, CT, g, as, eF, ir, cm, QA, Qt, bw, iP, eM, nB, iA, aep, aUS, k, Hb, m, Xu, ik, iu, aw, ht, n, fr, uE, rP, ft, jz, gd, KH, lG, eV, dL, kj, fQ, aq, kp, pm, cdT, ar, Sc, Qy, SE, dB, ayS, Be, auh, hL, E, uz, at, JM, it, B, dA, eR, jp, jx, eX, aA, aNG, lu, V, q, eA, nv, yg, aT, Fx, bq, rz, DX, bau, ae, dl, jf, Sh, s, nO, oV, Y, yL, r, aNi, en, aom, aDY, t, cl, btO, pb, Vi, bN, iK, ck, Qi, nM, iM, c, np, Bd, bt, zu, VB, ao, hS, kh, id, LX, bXc, jh, KO, Sv, cdS, Fc, iJ, que, ad, hX, dP, av, hd, ib, MS, py, Db, yt, hP, d, bug, je, PE, vj, ff, eH, gl, aF, iV, iR, adj, jb, bz, bF, es, gk, RV, bE, AR, adg, lf, X, y); #c1(MET) c2(NP_001120972) c3(hepatocyte growth factor receptor isoform a precursor) c4(45017) c5(45018, 45019, 45020, 45021, 45022, 45023, 45024, 45025, 45026, 45027, 45028, 45029) c6( ) c7(T, aC, aCh, pf, bll, al, bO, fd, bB, ci, aY, sD, lS, bv, aE, Xc, pX, Qk, ja, Vv, Ou, HR, K, hu, pk, eL, KC, eG, iE, Jl, aW, fc, CT, g, as, eF, ir, cm, QA, Qt, bw, iP, eM, nB, iA, aep, aUS, k, Hb, m, Xu, ik, iu, aw, ht, n, fr, uE, rP, ft, jz, gd, kH, lG, eV, dL, kj, fQ, aq, kp, pm, cdT, ar, Sc, Qy, SE, dB, ayS, Be, auh, HL, E, uz, at, JM, it, B, dA, eR, jp, jx, ex, aA, aNG, lu, V, q, eA, nv, yg, aT, Fx, bq, rz, DX, bau, ae, dl, jf, Sh, s, nO, oV, Y, yL, r, aNi, en, aom, aOY, t, cl, btO, pb, Vi, bN, iK, ck, Qi, nM, iM, c, np, Bd, bt, zu, VB, ao, hS, kh, id, LX, bXc, jh, KO, Sv, cdS, Fc, iJ, que, ad, hX, dP, av, hd, ib, MS, py, Db, yt, hP, d, bug, je, PE, vj, ff, eH, gl, aF, iV, iR, adj, jb, bz, bF, es, gk, RV, bE, AR, adg, lf, X, y); #c1(MET) c2(XP_006716053) c3(hepatocyte growth factor receptor isoform X2) c4( ) c5(290011, 290012, 290013, 290014, 290015, 290016) c6( ) c7(T, aC, aCh, pf, bll, al, bO, fd, bB, ci, aY, sD, lS, bv, aE, Xc, pX, Qk, ja, Vv, Du, HR, K, hu, pk, eL, KC, eG, iE, Jl, aW, fc, CT, g, as, eF, ir, cm, QA, Qt, bw, iP, eM, nB, iA, aep, aUS, k, Hb, m, Xu, iK, iu, aw, ht, n, fr, uE, rP, ft, jz, gd, KH, lG, eV, dL, kj, fQ, aq, kp, pm, cdT, ar, Sc, Qy, SE, dB, ayS, Be, auh, hL, E, uz, at, JM, it, B, dA, eR, jp, jx, eX, aA, aNG, lu, V, q, eA, nv, yg, aT, Fx, bq, rz, DX, bau, ae, dl, jf, Sh, s, nO, oV, Y, yL, r, aNi, en, aom, aOY, t, cl, btO, pb, Vi, bN, iK, ck, Qi, nM, iM, c, np, Bd, bt, zu, VB, ao, hS, kh, id, LX, bXc, jh, KO, Sv, cdS, Fc, iJ, aue, ad, hX, dP, av, hd, ib, MS, py, Db, yt, hP, d, bug, je, PE, vj, ff, eH, gl, aF, iV, iR, adj, jb, bz, bF, es, gk, RV, bE, AR, adg, lf, X, y); #c1(MET) c2(XP_011514525) c3(hepatocyte growth factor receptor isoform X1) c4(307223, 307224) c5(307225) c6( ) c7(T, aC, aCh, pf, bll, al, bO, fd, bB, ci, aY, sD, lS, bv, aE, Xc, pX, Qk, ja, Vv, Ou, HR, K, hu, pk, eL, KC, eG, iE, Jl, aW, fc, CT, g, as, eF, ir, cm, QA, Qt, bw, iP, eM, nB, iA, aep, aUS, k, Hb, m, Xu, ik, iu, aw, ht, n, fr, uE, rP, ft, jz, gd, kH, lG, eV, dL, kj, fQ, aq, kp, pm, cdT, ar, Sc, Qy, SE, dB, ayS, Be, auh, hL, E, uz, at, JM, it, B, dA, eR, jp, jx, eX, aA, aNG, lu, V, q, eA, nv, yg, aT, Fx, bq, rz, DX, bau, ae, dl, jf, Sh, s, nO, oV, Y, yL, r, aNi, en, aom, aDY, t, cl, btO, pb, Vi, bN, iK, ck, Qi, nM, iM, c, np, Bd, bt, zu, VB, ao, hS, kh, id, LX, bXc, jh, KO, Sv, cdS, Fc, iJ, aue, ad, hX, dP, av, hd, ib, MS, py, Db, yt, hP, d, bug, je, PE, vj, ff, eH, gl, aF, iV, iR, adj, jb, bz, bF, es, gk, RV, bE, AR, adg, lf, X, y); #c1(METAP1) c2(NP_055958) c3(methionine aminopeptidase 1) c4(184693) c5(184694, 184695, 184696, 184697, 184698, 184699) c6( ) c7(WP, J, aj); #c1(METAP1) c2(XP_005262925) c3(methionine aminopeptidase 1 isoform X4) c4(278166) c5(278167, 278168, 278169, 278170, 278171, 278172) c6( ) c7(WP, J, aj); #c1(METAP1D) c2(NP_954697) c3(methionine aminopeptidase 1D, mitochondrial precursor) c4(261275) c5(261276, 261277, 261278, 261279, 261280, 261281) c6( ) c7(bB, bE, X); #c1(METAP2) c2(NP_006829) c3(methionine aminopeptidase 2) c4(168497) c5(168498, 168499, 168500, 168501, 168502, 168503) c6( ) c7(ID, fM, fG, GC, V, eA, ib, dV, aT, h, ey, K, on, af, cp, aa, jf, ly, d, aW, Y, g, cm, lx, bN, LB, Qi, Ew, dc, w, adR, eo, X, L); #c1(METRN) c2(NP_076947) c3(meteorin precursor) c4(214345) c5(214346, 214347, 214348, 214349, 214350, 214351, 214352, 214353, 214354, 214355, 214356, 214357, 214358, 214359, 214360, 214361, 214362, 214363, 214364, 214365, 214366, 214367, 214368, 214369, 214370, 214371, 214372, 214373, 214374, 214375, 214376, 214377, 214378, 214379, 214380, 214381, 214382, 214383, 214384, 214385, 214386, 214387, 214388, 214389, 214390, 214391, 214392, 214393, 214394, 214395, 214396, 214397, 214398, 214399, 214400, 214401, 214402, 214403, 214404, 214405, 214406, 214407, 214408, 214409, 214410, 214411, 214412, 214413, 214414, 214415, 214416, 214417, 214418, 214419, 214420, 214421, 214422, 214423, 214424, 214425, 214426, 214427, 214428, 214429, 214430, 214431, 214432, 214433, 214434, 214435, 214436, 214437, 214438, 214439, 214440, 214441, 214442) c6( ) c7(aR, U); #c1(METTL1) c2(NP_005362) c3(tRNA (guanine-N(7)-)-methyltransferase isoform a) c4(155440) c5(155441, 155442, 155443, 155444, 155445, 155446) c6( ) c7(Xw, V, dL); #c1(METTL1) c2(NP_075422) c3(tRNA (guanine-N(7)-)-methyltransferase isoform c) c4(213859) c5(213860, 213861, 213862, 213863, 213864, 213865) c6( ) c7(Xw, V, dL); #c1(METTL13) c2(NP_001007240) c3(methyltransferase-like protein 13 isoform 3) c4(22594) c5(22595, 22596, 22597, 22598, 22599, 22600) c6( ) c7(iR, aq, T, B, jh); #c1(METTL13) c2(NP_057019) c3(methyltransferase-like protein 13 isoform 1) c4(188540, 188541, 188542) c5(188543, 188544, 188545, 188546, 188547, 188548, 188549, 188550, 188551, 188552, 188553, 188554, 188555, 188556, 188557, 188558, 188559, 188560) c6( ) c7(iR, aq, T, B, jh); #c1(METTL13) c2(XP_011507917) c3(methyltransferase-like protein 13 isoform X1) c4(298226) c5(298227, 298228, 298229, 298230, 298231, 298232) c6( ) c7(iR, aq, T, B, jh); #c1(METTL16) c2(NP_076991) c3(methyltransferase-like protein 16) c4(214649, 214650, 214651, 214652) c5(214653, 214654, 214655, 214656, 214657, 214658, 214659, 214660, 214661, 214662, 214663, 214664, 214665) c6( ) c7(ac, W); #c1(METTL16) c2(XP_011522311) c3(methyltransferase-like protein 16 isoform X1) c4( ) c5(316998, 316999, 317000, 317001, 317002, 317003, 317004, 317005, 317006, 317007, 317008) c6( ) c7(ac, W); #c1(METTL17) c2(NP_001025162) c3(methyltransferase-like protein 17, mitochondrial isoform 1 precursor) c4(29374, 29375) c5(29376, 29377, 29378, 29379, 29380, 29381, 29382) c6( ) c7(kC); #c1(METTL17) c2(NP_073571) c3(methyltransferase-like protein 17, mitochondrial isoform 2 precursor) c4(212883) c5(212884, 212885, 212886, 212887, 212888, 212889, 212890, 212891, 212892, 212893, 212894, 212895) c6( ) c7(kC); #c1(METTL18) c2(XP_011508450) c3(histidine protein methyltransferase 1 homolog isoform X1) c4(299170, 299171) c5(299172, 299173, 299174, 299175, 299176, 299177, 299178, 299179, 299180, 299181, 299182, 299183, 299184, 299185, 299186, 299187) c6( ) c7(bA, ey, M, cz); #c1(METTL21A) c2(NP_660323) c3(protein N-lysine methyltransferase METTL21A) c4(240913, 240914) c5(240915, 240916, 240917, 240918, 240919, 240920, 240921, 240922, 240923, 240924, 240925, 240926, 240927, 240928, 240929, 240930, 240931, 240932, 240933) c6( ) c7(fq); #c1(METTL21A) c2(XP_011509027) c3(protein N-lysine methyltransferase METTL21A isoform X3) c4( ) c5(299634, 299635, 299636) c6( ) c7(fq); #c1(METTL21A) c2(XP_011509033) c3(protein N-lysine methyltransferase METTL21A isoform X8) c4( ) c5(299637) c6( ) c7(fq); #c1(METTL21A) c2(XP_011509034) c3(protein N-lysine methyltransferase METTL21A isoform X9) c4( ) c5(299638, 299639, 299640, 299641, 299642, 299643, 299644, 299645, 299646) c6( ) c7(fq); #c1(METTL21B) c2(NP_056248) c3(protein-lysine methyltransferase METTL21B isoform a) c4(186713) c5(186714, 186715, 186716, 186717, 186718, 186719) c6( ) c7(V); #c1(METTL21B) c2(NP_996797) c3(protein-lysine methyltransferase METTL21B isoform b) c4(264560) c5(264561, 264562, 264563, 264564, 264565, 264566) c6( ) c7(V); #c1(METTL21C) c2(NP_001010977) c3(protein-lysine methyltransferase METTL21C) c4(24388) c5(24389, 24390, 24391, 24392, 24393, 24394) c6( ) c7(bA); #c1(METTL24) c2(NP_001116836) c3(methyltransferase-like protein 24 precursor) c4(43956) c5(43957, 43958, 43959, 43960, 43961, 43962, 43963, 43964) c6(43965, 43966) c7(aw); #c1(METTL6) c2(XP_005264927) c3(methyltransferase-like protein 6 isoform X2) c4( ) c5(279287, 279288, 279289, 279290, 279291, 279292, 279293, 279294, 279295, 279296, 279297, 279298, 279299, 279300, 279301, 279302, 279303, 279304, 279305, 279306, 279307, 279308, 279309) c6( ) c7(bz, aT); #c1(METTL6) c2(XP_006713032) c3(methyltransferase-like protein 6 isoform X1) c4(286974) c5(286975, 286976, 286977, 286978, 286979, 286980, 286981, 286982, 286983, 286984) c6( ) c7(bz, aT); #c1(METTL7A) c2(NP_054752) c3(methyltransferase-like protein 7A precursor) c4(177586, 177587, 177588) c5(177589, 177590, 177591, 177592, 177593, 177594, 177595, 177596, 177597, 177598, 177599, 177600, 177601, 177602, 177603, 177604, 177605, 177606, 177607, 177608, 177609, 177610, 177611, 177612, 177613, 177614, 177615, 177616, 177617, 177618) c6( ) c7(dP); #c1(METTL9) c2(NP_001070648) c3(methyltransferase-like protein 9 isoform 2 precursor) c4(34644) c5(34645, 34646, 34647, 34648, 34649, 34650) c6( ) c7(T, b, xX, if, aoA, qN, i, GR); #c1(METTL9) c2(NP_057109) c3(methyltransferase-like protein 9 isoform 1 precursor) c4(188952, 188953) c5(188954, 188955, 188956, 188957, 188958, 188959, 188960, 188961, 188962, 188963, 188964, 188965) c6( ) c7(T, b, xX, if, aoA, qN, i, GR); #c1(MEX3B) c2(NP_115622) c3(RNA-binding protein MEX3B) c4(223387) c5(223388, 223389, 223390, 223391, 223392, 223393) c6( ) c7(fq); #c1(MEX3C) c2(NP_057710) c3(RNA-binding E3 ubiquitin-protein ligase MEX3C) c4(192487) c5(192488, 192489, 192490, 192491, 192492, 192493) c6( ) c7(aw, cz, q, qa, r); #c1(MEX3D) c2(NP_001167589) c3(RNA-binding protein MEX3D isoform 2) c4(69344, 69345) c5(69346, 69347, 69348, 69349, 69350, 69351, 69352) c6( ) c7(ae, ad); #c1(MEX3D) c2(NP_976049) c3(RNA-binding protein MEX3D isoform 1) c4(262887) c5(262888, 262889, 262890, 262891, 262892, 262893, 262894, 262895, 262896, 262897, 262898) c6( ) c7(ae, ad); #c1(MFAP3) c2(NP_001128509) c3(microfibril-associated glycoprotein 3 isoform 2) c4( ) c5(50123, 50124, 50125, 50126, 50127) c6( ) c7(fq); #c1(MFAP3) c2(NP_001229265) c3 (microfibril-associated glycoprotein 3 isoform 1 precursor) c4(84065, 84066, 84067, 84068, 84069) c5(84070, 84071, 84072, 84073, 84074, 84075, 84076, 84077, 84078, 84079, 84080, 84081, 84082, 84083, 84084, 84085, 84086, 84087, 84088, 84089, 84090, 84091, 84092, 84093, 84094, 84095, 84096, 84097, 84098) c6( ) c7(fq); #c1(MFAP4) c2(NP_001185624) c3(microfibril-associated glycoprotein 4 isoform 1 precursor) c4(75990) c5(75991, 75992, 75993, 75994, 75995, 75996) c6( ) c7(t, e); #c1(MFAP4) c2(NP_002395) c3(microfibril-associated glycoprotein 4 isoform 2 precursor) c4(128092) c5(128093, 128094, 128095, 128096, 128097, 128098) c6( ) c7(t, e); #c1(MFAP5) c2(NP_001284638) c3(microfibrillar-associated protein 5 isoform b precursor) c4(112212) c5(112213, 112214, 112215, 112216, 112217, 112218, 112219, 112220, 112221, 112222, 112223, 112224, 112225, 112226) c6( ) c7(t, m); #c1(MFAP5) c2(NP_001284640) c3(microfibrillar-associated protein 5 isoform d precursor) c4(112227) c5(112228, 112229, 112230, 112231, 112232, 112233, 112234, 112235) c6( ) c7(t, m); #c1(MFAP5) c2(NP_003471) c3(microfibrillar-associated protein 5 isoform a precursor) c4(138600, 138601) c5(138602, 138603, 138604, 138605, 138606, 138607, 138608, 138609, 138610, 138611, 138612, 138613, 138614, 138615, 138616) c6(138617) c7(t, m); #c1(MFGE8) c2(NP_001108086) c3 (lactadherin isoform b precursor) c4(43092) c5(43093, 43094, 43095, 43096, 43097, 43098) c6( ) c7(bdo, T, dD, jp, qb, ea, bS, eq, C, mC, eA, aT, cy, dL, bK, bq, eL, aiB, aW, Y, ff, eZ, en, bN, aFb, Ei, U, uz, fL); #c1(MFGE8) c2(NP_005919) c3(lactadherin isoform a preproprotein) c4(160364) c5(160365, 160366, 160367, 160368, 160369, 160370) c6( ) c7(bdo, T, dD, jp, qb, ea, bS, eq, C, mC, eA, aT, cy, dL, bK, bq, eL, aiB, aW, Y, ff, eZ, en, bN, aFb, Ei, U, uz, fL); #c1(MFHAS1) c2(NP_004216) c3(malignant fibrous histiocytoma-amplified sequence 1) c4(144360) c5(144361, 144362, 144363, 144364, 144365, 144366) c6( ) c7(Gp, cdU, dt, fM, bX); #c1(MF12) c2(NP_005920) c3(melanotransferrin isoform 1 precursor) c4(160371) c5(160372, 160373, 160374, 160375, 160376, 160377) c6( ) c7(Mo, T, cdV, ac, nM, ya, Y, Ou, ci, be, Xh, af, cp, L); #c1(MF12) c2(NP_201573) c3(melanotransferrin isoform 2 precursor) c4(228279) c5(228280, 228281, 228282, 228283, 228284, 228285) c6( ) c7(Mo, T, cdV, ac, nM, ya, Y, Ou, ci, be, Xh, af, cp, L); #c1(MFN1) c2(XP_005247653) c3(mitofusin-1 isoform X1) c4(267893, 267894, 267895, 267896) c5(267897, 267898, 267899, 267900, 267901, 267902, 267903, 267904, 267905, 267906, 267907, 267908, 267909, 267910, 267911, 267912, 267913, 267914, 267915, 267916, 267917, 267918, 267919, 267920, 267921, 267922, 267923, 267924) c6( ) c7(T, bux, pZ, c, ae, ad); #c1(MFRP) c2(NP_113621) c3(membrane frizzled-related protein) c4(221774) c5(221775, 221776, 221777, 221778, 221779, 221780, 221781, 221782, 221783, 221784) c6( ) c7(nk, od, cdY, lR, nb, W, cdW, kj, bnP, sa, Be, cdZ, aqV, nw, v, DL, cdX, ei, nC, lJ); #c1(MFSD1) c2(NP_073573) c3(major facilitator superfamily domain-containing protein 1 isoform 1) c4(212903, 212904) c5(212905, 212906, 212907, 212908, 212909, 212910, 212911, 212912, 212913, 212914, 212915, 212916) c6( ) c7(ey); #c1(MFSD1) c2(XP_006713793) c3(major facilitator superfamily domain-containing protein 1 isoform X1) c4(287372, 287373) c5(287374, 287375, 287376, 287377, 287378, 287379, 287380, 287381, 287382, 287383, 287384, 287385) c6( ) c7(ey); #c1(MFSD11) c2(NP_001229466) c3(UNC93-like protein MFSD11 isoform 2) c4(84557) c5(84558, 84559, 84560, 84561, 84562, 84563) c6( ) c7(ey); #c1(MFSD11) c2(NP_077287) c3(UNC93-like protein MFSD11 isoform 1) c4(214948) c5(214949, 214950, 214951, 214952, 214953, 214954) c6( ) c7(ey); #c1(MFSD12) c2(NP_778148) c3(major facilitator superfamily domain-containing protein 12 isoform c) c4(251368) c5(251369, 251370, 251371, 251372, 251373, 251374) c6( ) c7(cm, T); #c1(MFSD12) c2(XP_006722710) c3(major facilitator superfamily domain-containing protein 12 isoform X1) c4(294847) c5(294848, 294849, 294850, 294851, 294852, 294853) c6( ) c7(cm, T); #c1(MFSD12) c2(XP_011525986) c3(major facilitator superfamily domain-containing protein 12 isoform X3) c4(320356) c5(320357, 320358, 320359, 320360, 320361, 320362) c6( ) c7(cm, T); #c1(MFSD6) c2(NP_060164) c3(major facilitator superfamily domain-containing protein 6) c4(194553, 194554, 194555) c5(194556, 194557, 194558, 194559, 194560, 194561, 194562, 194563, 194564, 194565, 194566, 194567, 194568, 194569, 194570, 194571, 194572, 194573, 194574, 194575, 194576, 194577, 194578, 194579, 194580, 194581, 194582) c6( ) c7(bN, bX); #c1(MFSD6) c2(XP_005246713) c3(major facilitator superfamily domain-containing protein 6 isoform X2) c4(267374, 267375, 267376) c5(267377, 267378, 267379, 267380, 267381, 267382, 267383, 267384, 267385, 267386, 267387, 267388, 267389) c6( ) c7(bN, bX); #c1(MFSD7) c2(NP_001281270) c3(major facilitator superfamily domain-containing protein 7 isoform 1) c4(111648) c5(111649, 111650, 111651, 111652, (11653, 111654) c6( ) c7(ci); #c1(MFSD7) c2(NP_001281271) c3(major facilitator superfamily domain-containing protein 7 isoform 3) c4(111655) c5(111656, 111657, 111658, 111659, 111660, 111661) c6( ) c7(ci); #c1(MFSD7) c2(NP_115595) c3(major facilitator superfamily domain-containing protein 7 isoform 2) c4(223311) c5(223312, 223313, 223314, 223315, 223316, 223317) c6( ) c7(ci); #c1(MFSD7) c2(XP_011511877) c3(major facilitator superfamily domain-containing protein 7 isoform X8) c4( ) c5(302601, 302602) c6( ) c7(ci); #c1(MFSD8) c2(NP_689991) c3(major facilitator superfamily domain-containing protein 8) c4(244666) c5(244667, 244668, 244669, 244670, 244671, 244672) c6( ) c7(U, Lq, rY, cea); #c1(MFSD8) c2(XP_005262957) c3(major facilitator superfamily domain-containing protein 8 isoform X8) c4(278175) c5(278176, 278177, 278178, 278179, 278180, 278181) c6( ) c7(U, Lq, rY, cea); #c1(MFSD8) c2(XP_011530132) c3(major facilitator superfamily domain-containing protein 8 isoform X2) c4( ) c5(326030) c6( ) c7(U, Lq, rY, cea); #c1(MFSD8) c2(XP_011530134) c3(major facilitator superfamily domain-containing protein 8 isoform X7) c4(326031) c5(326032, 326033, 326034, 326035, 326036) c6( ) c7(U, Lq, rY, cea); #c1(MGA) c2(NP_001074010) c3(MAX gene-associated protein isoform 2) c4(36937) c5(36938, 36939, 36940, 36941, 36942, 36943) c6( ) c7(ou, ft, aW, qK, cj, aT); #c1(MGA) c2(NP_001157745) c3(MAX gene-associated protein isoform 1) c4(62510, 62511) c5(62512, 62513, 62514, 62515, 62516, 62517, 62518, 62519, 62520, 62521, 62522, 62523, 62524) c6( ) c7(ou, ft, aW, qK, cj, aT); #c1(MGA) c2(XP_011519702) c3(MAX gene-associated protein isoform X11) c4( ) c5(314376) c6( ) c7(ou, ft, aW, qK, cj, aT); #c1(MGAM) c2(NP_004659) c3(maltase-glucoamylase, intestinal) c4(148589, 148590) c5(148591, 148592, 148593, 148594, 148595, 148596) c6( ) c7(T, ft, aW, ff, cj, aT, en); #c1(MGAM) c2(XP_011514975) c3(maltase-glucoamylase, intestinal isoform X1) c4(307774) c5(307775, 307776, 307777, 307778, 307779, 307780, 307781, 307782, 307783, 307784, 307785, 307786) c6( ) c7(T, ft, aW, ff, cj, al, en); #c1(MGARP) c2(NP_116012) c3(protein MGARP) c4(225205) c5(225206, 225207, 225208, 225209, 225210, 225211, 225212) c6( ) c7(y); #c1(MGAT1) c2(XP_011532865) c3 (alpha-1,3-mannosyl-glycoprotein 2-beta-N-acetylglucosaminyltransferase isoform X1) c4(328968, 328969, 328970, 328971, 328972, 328973, 328974, 328975, 328976, 328977, 328978) c5(328979, 328980, 328981, 328982, 328983, 328984, 328985, 328986, 328987, 328988, 328989, 328990, 328991, 328992, 328993, 328994, 328995, 328996, 328997, 328998, 328999, 329000, 329001, 329002, 329003, 329004, 329005, 329006, 329007, 329008, 329009, 329010, 329011, 329012, 329013, 329014, 329015, 329016, 329017, 329018, 329019, 329020, 329021, 329022, 329023, 329024, 329025, 329026, 329027, 329028, 329029, 329030, 329031, 329032, 329033, 329034, 329035, 329036, 329037, 329038, 329039, 329040, 329041, 329042, 329043, 329044, 329045, 329046, 329047, 329048, 329049, 329050, 329051, 329052, 329053, 329054, 329055, 329056, 329057, 329058, 329059, 329060) c6( ) c7(ke, T, A, aW, aT, gl, aj, aqm, v, Eg, V, ae, J, ad); #c1(MGAT2) c2(NP_002399) c3(alpha-1,6-mannosyl-glycoprotein 2-beta-N-acetylglucosaminyltransferase) c4(128106, 128107) c5(128108, 128109, 128110, 128111, 128112, 128113, 128114, 128115, 128116, 128117, 128118, 128119) c6( ) c7(dq, v, dv, ceb); #c1(MGAT3) c2(NP_001091740) c3(beta-1,4-mannosyl-glycoprotein 4-beta-N-acetylglucosaminyltransferase) c4(38722) c5(38723, 38724, 38725, 38726, 38727, 38728, 38729, 38730, 38731, 38732, 38733) c6( ) c7(aW, Y, al, aq, v, nJ, B, hX); #c1(MGAT3) c2(XP_005261665) c3(beta-1,4-mannosyl-glycoprotein 4-beta-N-acetylglucosaminyltransferase isoform X2) c4(277576) c5(277577) c6( ) c7(aW, Y, aT, aq, v, nJ, B, hX); #c1(MGAT3) c2(XP_006724315) c3(beta-1,4-mannosyl-glycoprotein 4-beta-N-acetylglucosaminyltransferase isoform X3) c4(295937) c5(295938, 295939, 295940, 295941, 295942, 295943) c6( ) c7(aW, Y, aT, aq, v, nJ, B, hX); #c1(MGAT4A) c2(NP_001153626) c3(alpha-1,3-mannosyl-glycoprotein 4-beta-N-acetylglucosaminyltransferase A isoform 2) c4(60785) c5(60786, 60787, 60788, 60789, 60790, 60791) c6( ) c7(T, aC, dq, v, bK, nJ); #c1(MGAT4A) c2(NP_036346) c3(alpha-1,3-mannosyl-glycoprotein 4-beta-N-acetylglucosaminyltransferase A isoform 1) c4(173111) c5(173112, 173113, 173114, 173115, 173116, 173117, 173118, 173119, 173120, 173121, 173122, 173123, 173124, 173125) c6( ) c7(T, aC, dq, v, bK, nJ); #c1(MGAT4B) c2(NP_055090) c3(alpha-1,3-mannosyl-glycoprotein 4-beta-N-acetylglucosaminyltransferase B isoform 1 precursor) c4(178880) c5(178881, 178882, 178883, 178884, 178885, 178886) c6( ) c7(nJ, T, aC); #c1(MGAT4B) c2(NP_463459) c3(alpha-1,3-mannosyl-glycoprotein 4-beta-N-acetylglucosaminyltransferase B isoform 2) c4(230382) c5(230383, 230384, 230385, 230386, 230387, 230388) c6( ) c7(nJ, T, aC); #c1(MGAT4C) c2(NP_037376) c3(alpha-1,3-mannosyl-glycoprotein 4-beta-N-acetylglucosaminyltransferase C) c4( ) c5(175113, 175114, 175115, 175116, 175117, 175118, 175119, 175120, 175121, 175122, 175123, 175124, 175125, 175126, 175127, 175128, 175129, 175130, 175131, 175132, 175133, 175134, 175135, 175136, 175137, 175138, 175139, 175140, 175141, 175142, 175143, 175144) c6( ) c7(aj); #c1(MGAT4C) c2(XP_005268833) c3(alpha-1,3-mannosyl-glycoprotein 4-beta-N-acetylglucosaminyltransferase C isoform X4) c4(282111) c5(282112, 282113, 282114, 282115, 282116, 282117, 282118, 282119, 282120, 282121) c6( ) c7(aj); #c1(MGAT4C) c2(XP_011536451) c3(alpha-1,3-mannosyl-glycoprotein 4-beta-N-acetylglucosaminyltransferase C isoform X2) c4(333815) c5( ) c6( ) c7(aj); #c1(MGAT4C) c2(XP_011536453) c3(alpha-1,3-mannosyl-glycoprotein 4-beta-N-acetylglucosaminyltransferase C isoform X3) c4(333816, 333817) c5(333818) c6( ) c7(aj); #c1(MGAT4C) c2(XP_011536457) c3(alpha-1,3-mannosyl-glycoprotein 4-beta-N-acetylglucosaminyltransferase C isoform X5) c4( ) c5(333819) c6( ) c7(aj); #c1(MGAT5) c2(XP_011509505) c3(alpha-1,6-mannosylglycoprotein 6-beta-N-acetylglucosaminyltransferase A isoform X1) c4(300075) c5(300076, 300077, 300078, 300079, 300080, 300081, 300082, 300083, 300084, 300085) c6( ) c7(T, dA, aA, bB, gf, ci, V, abC, ad, av, aYy, aT, dL, an, eu, B, s, Jl, aW, Y, fQ, alw, aF, iV, cm, fq, ac, bE, y, X); #c1(MGAT5B) c2(NP_001186101) c3(alpha-1,6-mannosylglycoprotein 6-beta-N-acetylglucosaminyltransferase B isoform 3) c4(77123) c5(77124, 77125, 77126, 77127, 77128, 77129) c6( ) c7(yt, T, kl, aF, aA); #c1(MGAT5B) c2(NP_653278) c3(alpha-1,6-mannosylglycoprotein 6-beta-N-acetylglucosaminyltransferase B isoform 1) c4(239483) c5(239484, 239485, 239486, 239487, 239488, 239489) c6( ) c7(yt, T, kl, aF, aA); #c1(MGAT5B) c2(NP_945193) c3(alpha-1,6-mannosylglycoprotein 6-beta-N-acetylglucosaminyltransferase B isoform 2) c4(260760) c5(260761, 260762, 260763, 260764, 260765, 260766) c6( ) c7(yt, T, kl, aF, aA); #c1(MGAT5B) c2(XP_011522655) c3(alpha-1,6-mannosylglycoprotein 6-beta-N-acetylglucosaminyltransferase B isoform X5) c4(317215) c5(317216, 317217, 317218, 317219, 317220, 317221) c6( ) c7(yt, T, kl, aF, aA); #c1(MGEA5) c2(NP_001135906) c3(protein O-GlcNAcase isoform b) c4(53531, 53532) c5(53533, 53534, 53535, 53536, 53537, 53538, 53539, 53540, 53541, 53542, 53543, 53544) c6( ) c7(bL, dD, bHJ, OI, ea, bDa, aw, cz, cQ, aj, v, vQ, eq, ww, q, J, cd, bHN, ad, bCZ, ip, bDz, gd, aT, lG, ae, cec, d, tb, aW, r, zC, Vk, vU, aDA, kg, bHI, tV, U, akH, awM, Lq, dy, mf, hh, bCS); #c1(MGEA5) c2(NP_036347) c3 (protein O-GlcNAcase isoform a) c4(173126) c5(173127, 173128, 173129, 173130, 173131, 173132, 173133, 173134, 173135, 173136, 173137, 173138) c6( ) c7(bL, dD, bHJ, OI, ea, bDa, aw, cz, cQ, aj, v, vQ, eq, ww, q, J, cd, bHN, ad, bCZ, ip, bDz, gd, aT, lG, ae, cec, d, tb, aW, r, zC, Vk, vU, aDA, kg, bHI, tV, U, akH, awM, Lq, dy, mf, hh, bCS); #c1(MGEA5) c2(XP_011537482) c3(protein O-GlcNAcase isoform X1) c4(335097) c5( ) c6( ) c7(bL, dD, bHJ, OI, ea, bDa, aw, cz, cQ, aj, v, vQ, eq, ww, q, J, cd, bHN, ad, bCZ, ip, bDz, gd, aT, lG, ae, cec, d, tb, aW, r, zC, Vk, vU, aDA, kg, bHI, tV, U, akH, awM, Lq, dy, mf, hh, bCS); #c1(MGEA5) c2(XP_011537484) c3(protein O-GlcNAcase isoform X3) c4(335098, 335099) c5(335100, 335101, 335102, 335103, 335104, 335105, 335106, 335107, 335108, 335109, 335110, 33511) c6( ) c7(bL, dD, bHJ, OI, ea, bDa, aw, cz, cQ, aj, v, vQ, eq, ww, q, J, cd, bHN, ad, bCZ, ip, bDz, gd, aT, lG, ae, cec, d, tb, aW, r, zC, Vk, vU, aDA, kg, bHI, tV, U, akH, awM, Lq, dy, mf, hh, bCS); #c1(MGLL) c2(NP_001003794) c3(monoglyceride lipase isoform 2) c4(20131, 20132) c5(20133, 20134, 20135, 20136, 20137, 20138, 20139, 20140, 20141, 20142, 20143, 20144, 20145, 20146, 20147) c6( ) c7(T, aiB, s, bE, aW, r, aT, bB, v, PQ, eA, jb, ae, q, ad, bA); #c1(MGLL) c2(NP_001243514) c3(monoglyceride lipase isoform 3) c4(90987) c5(90988, 90989, 90990, 90991, 90992, 90993, 90994, 90995, 90996, 90997, 90998, 90999) c6( ) c7(T, aiB, s, bE, aW, r, aT, bB, v, PQ, eA, jb, ae, q, ad, bA); #c1(MGLL) c2(NP_009214) c3(monoglyceride lipase isoform 1) c4(171546, 171547) c5(171548, 171549, 171550, 171551, 171552, 171553, 171554) c6( ) c7(T, aiB, s, bE, aW, r, aT, bB, v, PQ, eA, jb, ae, q, ad, bA); #c1(MGME1) c2(NP_443097) c3(mitochondrial genome maintenance exonuclease 1) c4(229402) c5(229403, 229404, 229405, 229406, 229407, 229408) c6( ) c7(AI, ced, kC); #c1(MGME1) c2(XP_011527697) c3(mitochondrial genome maintenance exonuclease 1 isoform X2) c4(322629) c5(322630, 322631, 322632, 322633, 322634, 322635) c6( ) c7(Al, ced, kC); #c1(MGMT) c2(NP_002403) c3(methylated-DNA-protein-cysteine methyltransferase) c4(128128) c5(128129, 128130, 128131, 128132, 128133, 128134) c6( ) c7(T, os, aC, fR, pf, if, al, fd, bB, amM, ci, ip, aE, Qk, K, hu, eL, cp, yo, aW, aEl, aoo, hl, LZ, nY, g, as, eF, bw, iP, eM, nB, Ip, iT, m, ik, QS, bGC, cE, sc, lT, ht, NT, qy, n, awr, qN, BI, ft, gd, bUP, Gw, dL, hB, oS, auM, GA, cM, fs, Ld, Zj, Wt, bJ, bP, asl, dB, hk, hL, hR, at, it, B, bL, fM, dA, aA, hy, bX, pd, q, eA, XX, lg, aT, jj, ae, bkl, jf, avg, s, n, Y, Lf, r, qL, en, bu, cl, t, iK, ck, kP, Dy, oX, ij, aws, bt, On, vi, GI, id, iW, YM, jh, OE, oZ, hF, Fc, ad, afb, hX, iC, av, hd, io, hP, ji, fb, d, BO, ff, eH, aF, VI, iV, bz, bF, gk, amS, Ew, bE, Ny, auH, Gu, HE, X); #c1(MGP) c2(NP_000891) c3(matrix G1a protein isoform 2 precursor) c4(16956) c5(16957, 16958, 16959, 16960, 16961, 16962, 16963) c6( ) c7(sY, T, dD, eR, ea, e, aw, eX, wo, aj, fo, j, ci, eq, fr, zP, q, J, Hr, fU, kl, aT, acb, eV, K, bA, zQ, d, cee, cW, Y, aW, ceg, r, rs, g, dw, uC, wU, cef, rR, rq, hO, aS, dy, lS, aAN); #c1(MGP) c2(NP_001177768) c3(matrix G1a protein isoform 1 precursor) c4(72998) c5(72999, 73000, 73001, 73002, 73003, 73004, 73005) c6( ) c7(sY, T, dD, eR, ea, e, aw, eX, wo, aj, fo, j, ci, eq, fr, zP, q, J, Hr, fU, kl, aT, acb, eV, K, bA, zQ, d, cee, cW, Y, aW, ceg, r, rs, g, dw, uC, wU, cef, rR, rq, hO, aS, dy, lS, aAN); #c1(MGRN1) c2(NP_001135761) c3(E3 ubiquitin-protein ligase MGRN1 isoform 2) c4( ) c5(53137) c6( ) c7(hk, Y, dt); #c1(MGRN1) c2(NP_001135762) c3(E3 ubiquitin-protein ligase MGRN1 isoform 3) c4(53138) c5(53139, 53140, 53141, 53142, 53143, 53144) c6( ) c7(hk, Y, dt); #c1(MGRN1) c2(NP_001135763) c3(E3 ubiquitin-protein ligase MGRN1 isoform 4) c4( ) c5(53145, 53146, 53147, 53148, 53149, 53150, 53151, 53152, 53153, 53154, 53155) c6( ) c7(hk, Y, dt); #c1(MGRN1) c2(NP_056061) c3(E3 ubiquitin-protein ligase MGRN1 isoform 1) c4(185466, 185467) c5(185468, 185469, 185470, 185471, 185472, 185473) c6( ) c7(hk, Y, dt); #c1(MGRN1) c2(XP_005255276) c3(E3 ubiquitin-protein ligase MGRN1 isoform X5) c4(273579) c5( ) c6( ) c7(hk, Y, dt); #c1(MGST1) c2(NP_001247440) c3(microsomal glutathione S-transferase 1 isoform a) c4(93629, 93630, 93631) c5(93632, 93633, 93634, 93635, 93636, 93637, 93638, 93639, 93640, 93641, 93642, 93643, 93644, 93645, 93646, 93647, 93648, 93649, 93650, 93651, 93652, 93653, 93654, 93655, 93656, 93657, 93658, 93659, 93660, 93661, 93662, 93663) c6( ) c7(GI, av, W, ff, U, r, K, en, cm, iK, bz, dx); #c1(MGST1) c2(NP_001254527) c3(microsomal glutathione S-transferase 1 isoform c) c4(94593) c5(94594, 94595, 94596, 94597, 94598, 94599, 94600, 94601, 94602, 94603, 94604, 94605) c6( ) c7(GI, av, W, ff, U, r, K, en, cm, iK, bz, dx); #c1(MGST1) c2(XP_011518975) c3(microsomal glutathione S-transferase 1 isoform X2) c4(313439) c5( ) c6( ) c7(GI, av, W, ff, U, r, K, en, cm, iK, bz, dx); #c1(MGST2) c2(NP_001191295) c3(microsomal glutathione S-transferase 2 isoform 1 precursor) c4(81333) c5(81334, 81335, 81336, 81337, 81338, 81339, 81340, 81341, 81342, 81343) c6( ) c7(aC, cr, iU, Jr); #c1(MGST2) c2(NP_001191297) c3(microsomal glutathione S-transferase 2 isoform 3 precursor) c4(81344) c5(81345, 81346, 81347, 81348, 81349, 81350) c6( ) c7(aC, cr, iU, Jr); #c1(MGST3) c2(NP_004519) c3(microsomal glutathione S-transferase 3) c4(147167, 147168) c5(147169, 147170, 147171, 147172, 147173, 147174, 147175, 147176, 147177, 147178, 147179, 147180, 147181, 147182, 147183, 147184, 147185, 147186, 147187, 147188, 147189, 147190, 147191, 147192, 147193, 147194, 147195, 147196) c6( ) c7(bL, af, bA, cz); #c1(MIA) c2(NP_001189482) c3(melanoma-derived growth regulatory protein precursor) c4(80217, 80218) c5(80219, 80220, 80221, 80222, 80223, 80224, 80225, 80226, 80227, 80228, 80229, 80230, 80231, 80232, 80233, 80234) c6( ) c7(T, kh, aC, aw, aA, j, ci, ale, qN, eA, eJ, iu, jz, aE, azh, cp, atX, d, Y, ff, kp, aF, en, g, bz, ck, bE, at, lf, B, y); #c1(MIA2) c2(NP_473365) c3(melanoma inhibitory activity protein 2 precursor) c4(230410, 230411) c5(230412, 230413, 230414, 230415, 230416, 230417, 230418, 230419, 230420, 230421, 230422, 230423, 230424, 230425, 230426, 230427) c6(230428, 230429, 230430, 230431) c7(F, ff, hl, aq, en, B); #c1(MIA3) c2(NP_001287796) c3(melanoma inhibitory activity protein 3 isoform 2) c4(112897, 112898, 112899) c5(112900, 112901, 112902, 112903, 112904, 112905, 112906, 112907, 112908, 112909, 112910, 112911, 112912, 112913, 112914, 112915, 112916) c6( ) c7(bL, dP, yo, A, Y, aw, mg, cj, mf, j, pz, B, lc); #c1(MIA3) c2(NP_940953) c3(melanoma inhibitory activity protein 3 isoform 1 precursor) c4(260181, 260182) c5(260183, 260184, 260185, 260186, 260187, 260188, 260189, 260190, 260191, 260192, 260193, 260194) c6( ) c7(bL, dP, yo, A, Y, aw, mg, cj, mf, j, pz, B, lc); #c1(MIA3) c2(XP_005273178) c3(melanoma inhibitory activity protein 3 isoform X1) c4(284326) c5( ) c6( ) c7(bL, dP, yo, A, Y, aw, mg, cj, mf, j, pz, B, lc); #c1(MIA3) c2(XP_006711367) c3(melanoma inhibitory activity protein 3 isoform X2) c4(285957, 285958) c5(285959, 285960, 285961, 285962, 285963, 285964, 285965, 285966, 285967, 285968, 285969, 285970) c6( ) c7(bL, dP, yo, A, Y, aw, mg, cj, mf, j, pz, B, lc); #c1(MIA3) c2(XP_011507815) c3(melanoma inhibitory activity protein 3 isoform X3) c4(297955) c5(297956, 297957, 297958, 297959, 297960, 297961) c6( ) c7(bL, dP, yo, A, Y, aw, mg, cj, mf, j, pz, B, lc); #c1(MIB1) c2(NP_065825) c3(E3 ubiquitin-protein ligase MIB1) c4(205699) c5(205700, 205701, 205702, 205703, 205704, 205705) c6( ) c7(T, apr, hV, fd, ci, ip, bv, gJ, MO, pk, eL, eG, akV, bPf, yo, aW, Ls, az, g, as, eF, avZ, vA, m, lT, Or, NT, cek, qy, n, fr, cc, dL, Yl, aiB, ge, aJG, YF, bfW, op, bP, dB, eD, KT, ceh, WL, B, aGn, fM, avm, aA, acn, cej, q, er, eA, RI, cei, nv, qK, aT, rz, ae, s, Y, qL, en, cl, t, KQ, QG, Dy, au, RW, aHB, zu, bt, VB, ava, kh, id, Dd, YM, bkL, jh, Fb, aFJ, Fc, iJ, ad, iC, bjn, Db, fb, apw, d, Hq, ff, eH, cr, Hp, bF, aoR, Ew, bhz, y); #c1(MIB2) c2(XP_011539035) c3(E3 ubiquitin-protein ligase MIB2 isoform X3) c4( ) c5(337121) c6( ) c7(Y, hd, fd, s, kH); #c1(MIB2) c2(XP_011539044) c3(E3 ubiquitin-protein ligase MIB2 isoform X12) c4( ) c5(337122, 337123, 337124) c6( ) c7(Y, hd, fd, s, kH); #c1(MICA) c2(NP_000238) c3(MHC class 1 polypeptide-related sequence A isoform 1 (MICA*001) precursor) c4(5336, 5337, 5338, 5339, 5340) c5(5341, 5342, 5343, 5344, 5345, 5346, 5347, 5348, 5349, 5350, 5351, 5352, 5353, 5354, 5355, 5356, 5357, 5358, 5359, 5360, 5361, 5362, 5363, 5364, 5365, 5366, 5367, 5368, 5369, 5370, 5371, 5372, 5373, 5374) c6( ) c7(T, if, cL, ci, aGX, amW, AW, aE, bFU, K, hH, avq, aW, asD, mQ, ih, hN, g, eF, cm, y, eM, Jr, nB, PV, m, P, bxC, aw, yG, n, aj, fr, qN, jz, lt, ju, bsg, h, eV, ajV, A, mR, oo, ge, fe, aq, acD, Zj, mg, Pr, B, bL, yk, jx, eX, aA, Qo, V, q, J, eA, bIC, aT, vH, Tj, hK, ae, oV, Y, r, afq, lo, en, iK, ck, sS, aYx, aaT, jE, ad, wD, hX, l, hd, od, hD, buh, d, lU, ff, eH, cr, eZ, aF, hp, iV, iR, bz, LB, bF, eL, hE, hv); #c1(MICA) c2(NP_001170990) c3(MHC class 1 polypeptide-related sequence A isoform 2 (MICA*00801) precursor) c4(69726, 69727, 69728, 69729) c5(69730, 69731, 69732, 69733, 69734, 69735, 69736, 69737, 69738, 69739, 69740, 69741, 69742, 69743, 69744, 69745, 69746, 69747, 69748, 69749, 69750, 69751, 69752, 69753, 69754, 69755, 69756, 69757, 69758, 69759, 69760, 69761, 69762, 69763, 69764, 69765, 69766, 69767, 69768, 69769, 69770, 69771, 69772) c6( ) c7(T, if, cL, ci, aGX, amW, AW, aE, bFU, K, hH, avq, aW, asD, mQ, ih, hN, g, eF, cm, y, eM, Jr, nB, PV, m, P, bxC, aw, yG, n, aj, fr, qN, jz, lt, ju, bsg, h, eV, ajV, A, mR, oo, ge, fe, aq, acD, Zj, mg, Pr, B, bL, yk, jx, eX, aA, Qo, V, q, J, eA, bIC, aT, vH, Tj, hK, ae, oV, Y, r, afq, lo, en, iK, ck, sS, aYx, aaT, jE, ad, wD, hX, l, hd, od, hD, buh, d, lU, ff, eH, cr, eZ, aF, hp, iV, iR, bz, LB, bF, eU, hE, hv); #c1(MICA) c2(NP_001276083) c3(MHC class 1 polypeptide-related sequence A isoform 4 (MICA*00801) c4(107890) c5(107891, 107892, 107893, 107894, 107895, 107896, 107897, 107898, 107899, 107900, 107901, 107902, 107903) c6( ) c7(T, if, cL, ci, aGX, amW, AW, aE, bFU, K, hH, avq, aW, asD, mQ, ih, hN, g, eF, cm, y, eM, Jr, nB, PV, m, P, bxC, aw, yG, n, aj, fr, qN, jz, lt, ju, bsg, h, eV, ajV, A, mR, oo, ge, fe, aq, acD, Zj, mg, Pr, B, bL, yk, jx, eX, aA, Qo, V, q, J, eA, bIC, aT, vH, Tj, hK, ae, oV, Y, r, afq, lo, en, iK, ck, sS, aYx, aaT, jE, ad, wD, hX, l, hd, od, hD, buh, d, IU, ff, eH, cr, eZ, aF, hp, iV, iR, bz, LB, bF, eU, hE, hv); #c1(MICAL2) c2(NP_001269592) c3(protein-methionine sulfoxide oxidase MICAL2 isoform a) c4(102108) c5(102109, 102110, 102111, 102112, 102113, 102114) c6( ) c7(bM, T, ac, aw, ae, ad); #c1(MICAL2) c2(NP_001269594) c3(protein-methionine sulfoxide oxidase MICAL2 isoform c) c4(102115) c5(102116, 102117, 102118, 102119, 102120, 102121) c6( ) c7(bM, T, ac, aw, ae, ad); #c1(MICAL2) c2(NP_001269595) c3(protein-methionine sulfoxide oxidase MICAL2 isoform d) c4(102122, 102123) c5(102124, 102125, 102126, 102127, 102128, 102129, 102130, 102131, 102132, 102133, 102134, 102135) c6( ) c7(bM, T, ac, aw, ae, ad); #c1(MICAL2) c2(NP_001269597) c3(protein-methionine sulfoxide oxidase MICAL2 isoform f) c4( ) c5(102136, 102137, 102138, 102139, 102140, 102141, 102142, 102143, 102144, 102145, 102146, 102147, 102148, 102149, 102150, 102151, 102152, 102153, 102154, 102155, 102156, 102157, 102158, 102159, 102160, 102161, 102162, 102163, 102164, 102165, 102166, 102167, 102168, 102169) c6( ) c7(bM, T, ac, aw, ae, ad); #c1(MICAL2) c2(XP_005253306) c3(protein-methionine sulfoxide oxidase MICAL2 isoform X1) c4(272423) c5(272424, 272425, 272426, 272427, 272428, 272429) c6( ) c7(bM, T, ac, aw, ae, ad); #c1(MICAL2) c2(XP_011518792) c3(protein-methionine sulfoxide oxidase MICAL2 isoform X2) c4(313195, 313196) c5(313197) c6( ) c7(bM, T, ac, aw, ae, ad); #c1(MICAL2) c2(XP_011518793) c3(protein-methionine sulfoxide oxidase MICAL2 isoform X3) c4(313198, 313199, 313200) c5( ) c6( ) c7(bM, T, ac, aw, ae, ad); #c1(MICALCL) c2(NP_116256) c3(MICAL C-terminal-like protein) c4(226005, 226006) c5(226007, 226008, 226009, 226010, 226011, 226012) c6( ) c7(EQ); #c1(MICB) c2(NP_001276090) c3(MHC class 1 polypeptide-related sequence B isoform 3 precursor) c4(107923, 107924, 107925, 107926, 107927, 107928, 107929, 107930) c5(107931, 107932, 107933, 107934, 107935, 107936, 107937, 107938, 107939, 107940, 107941, 107942, 107943, 107944, 107945, 107946, 107947, 107948, 107949, 107950, 107951, 107952, 107953, 107954, 107955, 107956, 107957, 107958, 107959, 107960, 107961, 107962, 107963, 107964, 107965, 107966, 107967, 107968, 107969, 107970, 107971, 107972, 107973, 107974, 107975, 107976, 107977, 107978, 107979, 107980, 107981, 107982, 107983, 107984, 107985, 107986, 107987, 107988, 107989, 107990, 107991, 107992, 107993, 107994) c6( ) c7(bM, T, fR, if, jx, fd, gA, qN, V, q, wD, AW, cc, dV, aT, ja, K, hH, af, buh, atJ, A, aW, eH, cr, r, eZ, hN, aDm, hp, iV, eF, acD, eU, iQ, AH, gZ, fL, y, B); #c1(MICB) c2(NP_005922) c3(MHC class 1 polypeptide-related sequence B isoform 1 precursor) c4(160393, 160394, 160395, 160396, 160397, 160398, 160399) c5(160400, 160401, 160402, 160403, 160404, 160405, 160406, 160407, 160408, 160409, 160410, 160411, 160412, 160413, 160414, 160415, 160416, 160417, 160418, 160419, 160420, 160421, 160422, 160423, 160424, 160425, 160426, 160427, 160428, 160429, 160430, 160431, 160432, 160433, 160434, 160435, 160436, 160437, 160438, 160439, 160440, 160441, 160442, 160443, 160444, 160445, 160446) c6( ) c7(bM, T, fR, if, jx, fd, gA, qN, V, q, wD, AW, cc, dV, aT, ja, K, hH, af, buh, atJ, A, aW, eH, cr, r, eZ, hN, aDm, hp, iV, eF, acD, eU, iQ, AH, gZ, fL, y, B); #c1(MICB) c2(XP_011512933) c3(MHC class 1 polypeptide-related sequence B isoform X1) c4( ) c5(304332, 304333, 304334, 304335, 304336, 304337, 304338, 304339) c6( ) c7(bM, T, fR, if, jx, fd, gA, qN, V, q, wD, AW, cc, dV, aT, ja, K, hH, af, buh, atJ, A, aW, eH, cr, r, eZ, hN, aDm, hp, iV, eF, acD, eU, iQ, AH, gZ, fL, y, B); #c1(MICB) c2(XP_011545554) c3(MHC class 1 polypeptide-related sequence B isoform X1) c4(344323) c5(344324, 344325, 344326, 344327, 344328) c6( ) c7(bM, T, fR, if, jx, fd, gA, qN, V, q, wD, AW, cc, dV, aT, ja, K, hH, af, buh, atJ, A, aW, eH, cr, r, eZ, hN, aDm, hp, iV, eF, acD, eU, iQ, AH, gZ, fl, y, B); #c1(MICB) c2(XP_011545958) c3(MHC class 1 polypeptide-related sequence B isoform X1) c4(344802) c5(344803, 344804, 344805, 344806, 344807) c6( ) c7(bM, T, fR, if, jx, fd, gA, qN, V, q, wD, AW, cc, dV, aT, ja, K, hH, af, buh, atJ, A, aW, eH, cr, r, eZ, hN, aDm, hp, iV, eF, acD, eU, iQ, AH, gZ, fL, y, B); #c1(MICB) c2(XP_011546355) c3(MHC class 1 polypeptide-related sequence B isoform X1) c4(345070) c5(345071, 345072, 345073, 345074, 345075) c6( ) c7(bM, T, fR, if, jx, fd, gA, qN, V, q, wD, AW, cc, dV, aT, ja, K, hH, af, buh, atJ, A, aW, eH, cr, r, eZ, hN, aDm, hp, iV, eF, acD, eU, iQ, AH, gZ, fL, y, B); #c1(MICU1) c2(NP_001182447) c3(calcium uptake protein 1, mitochondrial isoform 2) c4(74920, 74921) c5(74922, 74923, 74924, 74925, 74926, 74927, 74928, 74929, 74930, 74931, 74932, 74933, 74934) c6( ) c7(T, nv, bht, xs, oj, bcD, cel, xw, xt); #c1(MICU1) c2(NP_001182448) c3(calcium uptake protein 1, mitochondrial isoform 3) c4(74935, 74936) c5(74937, 74938, 74939, 74940, 74941, 74942, 74943, 74944, 74945, 74946, 74947) c6( ) c7(T, nv, bht, xs, oj, bcD, cel, xw, xt); #c1(MICU1) c2(NP_006068) c3(calcium uptake protein 1, mitochondrial isoform 1) c4(161642) c5(161643, 161644, 161645, 161646, 161647, 161648) c6( ) c7(T, nv, bht, xs, oj, bcD, cel, xw, xt); #c1(MICU3) c2(NP_859074) c3(calcium uptake protein 3, mitochondrial) c4(255508) c5(255509, 255510, 255511, 255512, 255513, 255514) c6( ) c7(cm, sj); #c1(MICU3) c2(XP_006716391) c3(calcium uptake protein 3, mitochondrial isoform X3) c4( ) c5(290274) c6( ) c7(cm, sj); #c1(MID1) c2(NP_001180206) c3(E3 ubiquitin-protein ligase Midline-1 isoform 1) c4(73377, 73378, 73379) c5(73380, 73381, 73382, 73383, 73384, 73385, 73386, 73387, 73388, 73389, 73390, 73391, 73392, 73393, 73394, 73395, 73396, 73397, 73398, 73399, 73400, 73401, 73402, 73403, 73404, 73405, 73406, 73407, 73408, 73409, 73410, 73411, 73412, 73413, 73414, 73415, 73416, 73417, 73418) c6( ) c7(Na, T, aml, nX, eH, cem, aRk, Nc, Pp, ci, K, eF, ae, ad); #c1(MID1) c2(NP_001180208) c3(E3 ubiquitin-protein ligase Midline-I isoform 4) c4( ) c5(73419, 73420, 73421, 73422, 73423, 73424) c6( ) c7(Na, T, aml, nX, eH, cem, aRk, Nc, Pp, ci, K, eF, ae, ad); #c1(MID1) c2(NP_150631) c3(E3 ubiquitin-protein ligase Midline-1 isoform 2) c4(228150) c5(228151, 228152, 228153, 228154, 228155, 228156) c6( ) c7(Na, T, aml, nX, eH, cem, aRk, Nc, Pp, ci, K, ef, ae, ad); #c1(MID1) c2(XP_005274595) c3(E3 ubiquitin-protein ligase Midline-1 isoform X1) c4(285173) c5( ) c6( ) c7(Na, T, aml, nX, eH, cem, aRk, Nc, Pp, ci, K, eF, ae, ad); #c1(MID2) c2(NP_036348) c3(probable E3 ubiquitin-protein ligase MID2 isoform 1) c4(173139, 173140) c5(173141, 173142, 173143, 173144, 173145, 173146, 173147) c6( ) c7(aml, m, aRk); #c1(MID2) c2(NP_438112) c3(probable E3 ubiquitin-protein ligase MID2 isoform 2) c4(229105) c5(229106, 229107, 229108, 229109, 229110, 229111, 229112, 229113, 229114, 229115, 229116, 229117) c6( ) c7(aml, m, aRk); #c1(MIEN1) c2(NP_115715) c3(migration and invasion enhancer 1) c4(223687) c5(223688, 223689, 223690, 223691, 223692, 223693) c6( ) c7(T, aW, aA, aT, AO, in, ae, ad); #c1(MIER2) c2(NP_060020) c3(mesoderm induction early response protein 2) c4(193711) c5(193712, 193713, 193714, 193715, 193716, 193717) c6( ) c7(qK); #c1(MIER3) c2(NP_001284528) c3(mesoderm induction early response protein 3 isoform 2) c4(111973) c5(111974, 111975, 111976, 111977, 111978, 111979) c6( ) c7(aW, T, aT); #c1(MIER3) c2(NP_689835) c3(mesoderm induction early response protein 3 isoform 3) c4(244100) c5(244101, 244102, 244103, 244104, 244105, 244106) c6( ) c7(aW, T, aT); #c1(MIF) c2(NP_002406) c3(macrophage migration inhibitory factor) c4(128143) c5(128144, 128145, 128146, 128147, 128148, 128149) c6( ) c7(bM, T, xS, fR, ea, e, bB, cL, lR, ci, bh, ip, rJ, bAy, dV, K, hH, eL, bc, Us, hm, aa, UC, yo, zQ, aAg, aW, az, HA, ih, fc, kt, pD, aB, g, eE, cm, uC, Qn, fT, eM, Jr, aiM, cj, U, uJ, k, m, Uz, eg, aFf, qR, aZt, dD, aw, aj, ct, zP, bPL, ai, cc, SH, dL, wb, ey, A, apo, EU, lb, ge, fQ, fe, bRD, hq, ar, dB, afc, bm, at, my, fL, eo, Pr, B, bL, pF, eR, aA, cz, fF, v, j, C, Qo, V, eA, J, o, jZ, qp, qK, aT, cy, ae, kl, s, Y, fO, brW, lo, t, iK, pb, bN, OW, ua, c, ac, ig, hc, AH, dy, EY, fD, ao, sY, kh, akd, aD, dM, ha, jh, WV, ah, jE, eq, mC, Uy, ad, hX, dP, av, ot, ds, oW, hD, ji, lQ, jo, uQ, d, cH, KS, cr, eZ, dN, brp, aF, hp, Fq, iV, D, bE, wz, aEs, mz, XO, ME, lf, iu, y); #c1(MILR1) c2(XP_011522929) c3 (allergin-1 isoform X1) c4(317428) c5(317429, 317430, 317431, 317432, 317433, 317434) c6( ) c7(Vk); #c1(MINA) c2(NP_116167) c3(bifunctional lysine-specific demethylase and histidyl-hydroxylase MINA isoform b) c4(225516, 225517) c5(225518, 225519, 225520, 225521, 225522, 225523, 225524, 225525, 225526, 225527, 225528, 225529, 225530, 225531, 225532, 225533, 225534, 225535) c6( ) c7(jf, av, T, s, bE, aW, Qu, ff, jh, aT, bB, aF, fr, ci, en, g, bz, X); #c1(MINA) c2(NP_694822) c3(bifunctional lysine-specific demethylase and histidyl-hydroxylase MINA isoform a) c4(245378, 245379) c5(245380, 245381, 245382, 245383, 245384, 245385, 245386, 245387, 245388, 245389) c6( ) c7(jf, av, T, s, bE, aW, Qu, ff, jh, aT, bB, aF, fr, ci, en, g, bz, X); #c1(MINA) c2(XP_011511546) c3(bifunctional lysine-specific demethylase and histidyl-hydroxylase MINA isoform X2) c4(302250) c5(302251, 302252, 302253, 302254, 302255, 302256) c6( ) c7(jf, av, T, s, bE, aW, Qu, ff, jh, aT, bB, aF, fr, ci, en, g, bz, X); #c1(MINK1) c2(NP_001020108) c3(misshapen-like kinase 1 isoform 4) c4(28172) c5(28173, 28174, 28175, 28176, 28177, 28178) c6( ) c7(T, eG, mr, wL, Y, gZ, h, aLJ, K, act, MC, eW, mu); #c1(MINK1) c2(NP_056531) c3(misshapen-like kinase 1 isoform 1) c4( ) c5(187970) c6( ) c7(T, eG, mr, wL, Y, gZ, h, aLJ, K, act, MC, eW, mu); #c1(MINK1) c2(NP_722549) c3(misshapen-like kinase 1 isoform 3) c4(246829) c5(246830, 246831, 246832, 246833, 246834, 246835) c6( ) c7(T, eG, mr, wL, Y, gZ, h, aLJ, K, act, MC, eW, mu); #c1(MINK1) c2(NP_733763) c3(misshapen-like kinase 1 isoform 2) c4(246990) c5(246991, 246992, 246993, 246994, 246995, 246996) c6( ) c7(T, eG, mr, wL, Y, gZ, h, aLJ, K, act, MC, eW, mu); #c1(MINK1) c2(XP_005256721) c3(misshapen-like kinase 1 isoform X1) c4( ) c5(274304) c6( ) c7(T, eG, mr, wL, Y, gZ, h, aLJ, K, act, MC, eW, mu); #c1(MINK1) c2(XP_006721593) c3(misshapen-like kinase 1 isoform X2) c4(294109) c5( ) c6( ) c7(T, eG, mr, wL, Y, gZ, h, aLJ, K, act, MC, eW, mu); #c1(MINK1) c2(XP_006721598) c3(misshapen-like kinase 1 isoform X9) c4(294110) c5( ) c6( ) c7(T, eG, mr, wL, Y, gZ, h, aLJ, K, act, MC, eW, mu); #c1(MINK1) c2(XP_011522210) c3(misshapen-like kinase 1 isoform X14) c4( ) c5(316826, 316827, 316828, 316829, 316830, 316831, 316832, 316833, 316834, 316835, 316836) c6( ) c7(T, eG, mr, wL, Y, gZ, h, aLJ, K, act, MC, eW, mu); #c1(MINK1) c2(XP_011522211) c3(misshapen-like kinase 1 isoform X15) c4(316837) c5(316838, 316839, 316840, 316841) c6( ) c7(T, eG, mr, wL, Y, gZ, h, aLJ, K, act, MC, eW, mu); #c1(MINPP1) c2(NP_001171588) c3(multiple inositol polyphosphate phosphatase 1 isoform 2 precursor) c4(70663) c5(70664, 70665, 70666, 70667, 70668, 70669) c6( ) c7(qJ, nB, fd, ey, ci, lQ, Pk, aMI); #c1(MINPP1) c2(NP_004888) c3(multiple inositol polyphosphate phosphatase 1 isoform 1 precursor) c4(150564, 150565) c5(150566, 150567, 150568, 150569, 150570, 150571, 150572, 150573, 150574, 150575, 150576, 150577) c6( ) c7(qJ, nB, fd, ey, ci, lQ, Pk, aMI); #c1(MINPP1) c2(XP_011538681) c3(multiple inositol polyphosphate phosphatase 1 isoform X1) c4(336423) c5(336424, 336425, 336426, 336427, 336428, 336429) c6( ) c7(qJ, nB, fd, ey, ci, lQ, Pk, aMI); #c1(MIOX) c2(NP_060054) c3(inositol oxygenase) c4(193894, 193895, 193896) c5(193897, 193898, 193899, 193900, 193901, 193902, 193903, 193904, 193905, 193906, 193907, 193908, 193909, 193910, 193911, 193912, 193913, 193914, 193915) c6( ) c7(aj, A); #c1(MIOX) c2(XP_011529007) c3 (inositol oxygenase isoform X2) c4(324363) c5(324364, 324365, 324366, 324367, 324368) c6( ) c7(aj, A); #c1(MIP) c2(NP_036196) c3(lens fiber major intrinsic protein) c4(172337) c5(172338, 172339, 172340, 172341, 172342, 172343) c6( ) c7(bM, T, Dw, dA, aD, fG, lH, bLS, jh, fd, aj, j, Zh, ci, UX, fr, pz, q, J, cd, aoH, aXI, eW, zw, aXC, xE, aWc, arm, M, kH, cU, aT, dV, h, mJ, azh, cen, ceo, nP, je, caK, bf, aW, xz, r, fe, aq, iV, Bu, QA, pb, aBs, LB, ck, pa, iM, aDA, bE, wO, hc, aS, fD, bwg, y, bt, B); #c1(MIPEP) c2(NP_005923) c3(mitochondrial intermediate peptidase) c4(160447) c5(160448, 160449, 160450, 160451, 160452, 160453) c6( ) c7(bM, T, Dw, dA, aD, fG, lH, bLS, jh, fd, aj, j, ci, UX, fr, pz, q, J, cd, aXI, aoH, eW, zw, aXC, xE, aWc, M, kH, cU, aT, dV, h, mJ, azh, ceo, je, caK, bf, aW, xz, r, fe, aq, iV, Bu, bz, pb, QA, aBs, LB, ck, pa, iM, aDA, bE, wO, hc, Be, aS, fD, bwg, y, bt, B); #c1(MIPOL1) c2(NP_620059) c3(mirror-image polydactyly gene 1 protein) c4(237203, 237204, 237205) c5(237206, 237207, 237208, 237209, 237210, 237211, 237212, 237213, 237214, 237215, 237216, 237217, 237218, 237219, 237220, 237221, 237222, 237223, 237224, 237225) c6( ) c7(zG, bGy); #c1(MIPOL1) c2(XP_011534766) c3(mirror-image polydactyly gene 1 protein isoform X3) c4(331203, 331204) c5( ) c6( ) c7(zG, bGy); #c1(MIPOL1) c2(XP_011534767) c3(mirror-image polydactyly gene 1 protein isoform X4) c4( ) c5(331205, 331206, 331207, 331208, 331209, 331210) c6( ) c7(zG, bGy); #c1(MIPOL1) c2(XP_011534769) c3(mirror-image polydactyly gene 1 protein isoform X7) c4( ) c5(331211, 331212, 331213, 331214, 331215, 331216, 331217, 331218, 331219, 331220, 331221, 331222) c6( ) c7(zG, bGy); #c1(MIR1-IHG) c2(NP_848558) c3(uncharacterized protein MIR1-IHG) c4(253842, 253843) c5(253844, 253845, 253846, 253847, 253848, 253849, 253850, 253851, 253852, 253853, 253854, 253855, 253856, 253857, 253858, 253859) c6( ) c7(cz); #c1(MIS18BP1) c2(XP_005267890) c3(mis18-binding protein 1 isoform X1) c4(281266, 281267) c5(281268, 281269, 281270, 281271, 281272, 281273, 281274, 281275, 281276, 281277, 281278, 281279, 281280) c6( ) c7(EW, j, aj); #c1(MITF) c2(NP_000239) c3(microphthalmia-associated transcription factor isoform 4) c4(5375) c5(5376, 5377, 5378, 5379, 5380, 5381, 5382) c6( ) c7(T, iW, cet, cep, amj, qj, ci, bjN, fr, pC, eA, aR, aJe, aJO, aJf, aE, K, Xh, ahE, cp, lR, kj, oV, Y, Tc, ahI, aTa, adj, dr, yR, ces, arv, es, Ta, iP, Dy, iA, hk, mG, cer, y, ceq); #c1(MITF) c2(NP_001171896) c3(microphthalmia-associated transcription factor isoform 7) c4( ) c5(71979, 71980, 71981, 71982, 71983, 71984, 71985, 71986, 71987, 71988, 71989, 71990) c6( ) c7(T, iW, cet, cep, amj, qj, ci, bjN, fr, pC, eA, aR, ale, aJO, aJf, aE, K, Xh, ahE, cp, lR, kj, oV, Y, Tc, ahI, aTa, adj, dr, yR, ces, arv, es, Ta, iP, Dy, iA, hk, mG, cer, y, ceq); #c1(MITF) c2(NP_006713) c3(microphthalmia-associated transcription factor isoform 3) c4(167349, 167350, 167351) c5(167352, 167353, 167354, 167355, 167356, 167357, 167358, 167359, 167360, 167361, 167362, 167363) c6( ) c7(T, iW, cet, cep, amj, qj, ci, bjN, fr, pC, eA, aR, aJe, aJO, aJf, aE, K, Xh, ahE, cp, lR, kj, oV, Y, Tc, ahI, aTa, adj, dr, yR, ces, arv, es, Ta, iP, Dy, iA, hk, mG, cer, y, ceq); #c1(MITF) c2(NP_937801) c3 (microphthalmia-associated transcription factor isoform 5) c4(259139) c5(259140, 259141, 259142, 259143, 259144, 259145) c6( ) c7(T, iW, cet, cep, amj, qj, ci, bjN, fr, pC, eA, aR, aJe, aJO, aJf, aE, K, Xh, ahE, cp, lR, kj, oV, Y, Tc, ahI, aTa, adj, dr, yR, ces, arv, es, Ta, iP, Dy, iA, hk, mG, cer, y, ceq); #c1(MITF) c2(NP_937802) c3(microphthalmia-associated transcription factor isoform 1) c4(259146, 259147) c5(259148, 259149, 259150, 259151, 259152, 259153) c6( ) c7(T, iW, cet, cep, amj, qj, ci, bjN, fr, pC, eA, aR, aJe, aJO, aJf, aE, K, Xh, ahE, cp, lR, kj, oV, Y, Tc, ahI, aTa, adj, dr, yR, ces, arv, es, Ta, iP, Dy, iA, hk, mG, cer, y, ceq); #c1(MITF) c2(NP_937820) c3(microphthalmia-associated transcription factor isoform 2) c4(259178) c5(259179, 259180, 259181, 259182, 259183, 259184) c6( ) c7(T, iW, cet, cep, amj, qj, ci, bjN, fr, pC, eA, aR, aJe, aJO, aJf, aE, K, Xh, ahE, cp, lR, kj, oV, Y, Tc, ahI, aTa, adj, dr, yR, ces, arv, es, Ta, iP, Dy, iA, hk, mG, cer, y, ceq); #c1(MITF) c2(NP_937821) c3(microphthalmia-associated transcription factor isoform 6) c4(259185) c5(259186, 259187, 259188, 259189, 259190, 259191) c6( ) c7(T, iW, cet, cep, amj, qj, ci, bjN, fr, pC, eA, aR, aJe, aJO, aJf, aE, K, Xh, ahE, cp, lR, kj, oV, Y, Tc, ahI, aTa, adj, dr, yR, ces, arv, es, Ta, iP, Dy, iA, hk, mG, cer, y, ceq); #c1(MITF) c2(XP_005264811) c3(microphthalmia-associated transcription factor isoform XI) c4(279255) c5(279256, 279257, 279258, 279259, 279260, 279261) c6( ) c7(T, iW, cet, cep, amj, qj, ci, bjN, fr, pC, eA, aR, ale, aJO, aJf, aE, K, Xh, ahE, cp, lR, kj, oV, Y, Tc, ahI, aTa, adj, dr, yR, ces, arv, es, Ta, iP, Dy, iA, hk, mG, cer, y, ceq); #c1(MITF) c2(XP_005264812) c3(microphthalmia-associated transcription factor isoform X3) c4(279262) c5(279263, 279264, 279265, 279266, 279267, 279268) c6( ) c7(T, iW, cet, cep, amj, qj, ci, bjN, fr, pC, eA, aR, aJe, aJO, aJf, aE, K, Xh, ahE, cp, lR, kj, oV, Y, Tc, ahI, aTa, adj, dr, yR, ces, arv, es, Ta, iP, Dy, iA, hk, mG, cer, y, ceq); #c1(MIXL1) c2(NP_001269331) c3(homeobox protein MIXL1 isoform 1) c4(101148) c5(101149, 101150, 101151, 101152, 101153, 101154) c6( ) c7(iW, ex, jN, jh, eV, uJ, hf, eE, pk); #c1(MIXL1) c2(NP_114150) c3(homeobox protein MIXL1 isoform 2) c4(222353) c5(222354, 222355, 222356, 222357, 222358, 222359, 222360) c6( ) c7(iW, ex, jN, jh, eV, uJ, hf, eE, pk); #c1(MKI67) c2(NP_001139438) c3(antigen KI-67 isoform 2) c4(59358, 59359) c5(59360, 59361, 59362, 59363, 59364, 59365, 59366, 59367, 59368, 59369, 59370, 59371) c6( ) c7(T, apr, hV, e, ci, ip, bv, gJ, Bc, K, MO, pk, eL, eG, akV, bPf, yo, aW, Ls, az, LZ, Qm, g, as, eF, bkD, eM, nB, vA, cj, apy, m, iK, lT, zX, Or, aw, ht, NT, cek, qy, n, fr, ft, dL, ey, Yl, aiB, ge, aJG, yq, mo, YF, op, bP, dB, eD, KT, B, bL, aGn, fM, avm, jx, aA, hy, j, acn, bX, q, eA, RI, cei, qK, aT, rz, ae, eu, s, Y, qL, en, cl, t, KQ, QG, Dy, ac, au, RW, aHB, aJQ, dy, zu, bt, VB, kh, id, Dd, YM, bkL, jh, Fb, aFJ, Fc, iJ, ad, iC, av, ib, bjn, Db, NS, fb, apw, d, qt, ff, eH, cr, Hp, bF, es, aoR, Ew, bhz, y, X); #c1(MKI67) c2(NP_002408) c3 (antigen KI-67 isoform 1) c4(128157, 128158, 128159) c5(128160, 128161, 128162, 128163, 128164, 128165, 128166, 128167, 128168, 128169, 128170, 128171, 128172, 128173, 128174, 128175, 128176) c6( ) c7(T, apr, hV, e, ci, ip, bv, gJ, Bc, K, MO, pk, eL, eG, akV, bPf, yo, aW, Ls, az, LZ, Qm, g, as, eF, bkD, eM, nB, vA, cj, apy, m, iK, lT, zX, Or, aw, ht, NT, cek, qy, n, fr, ft, dL, ey, Yl, aiB, ge, aJG, yq, mo, YF, op, bP, dB, eD, KT, B, bL, aGn, fM, avm, jx, aA, hy, j, acn, bX, q, eA, RI, cei, qK, aT, rz, ae, eu, s, Y, qL, en, cl, t, KQ, QG, Dy, ac, au, RW, aHB, aJQ, dy, zu, bt, VB, kh, id, Dd, YM, bkL, jh, Fb, aFJ, Fc, iJ, ad, iC, av, ib, bjn, Db, NS, fb, apw, d, qt, ff, eH, cr, Hp, bF, es, aoR, Ew, bhz, y, X); #c1(MKI67) c2(XP_006717927) c3(antigen KI-67 isoform X2) c4( ) c5(291393) c6( ) c7(T, apr, hV, e, ci, ip, bv, gJ, Bc, K, MO, pk, eL, eG, akV, bPf, yo, aW, Ls, az, LZ, Qm, g, as, eF, bkD, eM, nB, vA, cj, apy, m, iK, lT, zX, Or, aw, ht, NT, cek, qy, n, fr, ft, dL, ey, YI, aiB, ge, aJG, yq, mo, YF, op, bP, dB, eD, KT, B, bL, aGn, fM, avm, jx, aA, hy, j, acn, bX, q, eA, RI, cei, qK, aT, rz, ae, eu, s, Y, qL, en, cl, t, KQ, QG, Dy, ac, au, RW, aHB, aJQ, dy, zu, bt, VB, kh, id, Dd, YM, bkL, jh, Fb, aFJ, Fc, iJ, ad, iC, av, ib, bjn, Db, NS, fb, apw, d, qt, ff, eH, cr, Hp, bF, es, aoR, Ew, bhz, y, X); #c1(MKKS) c2(NP_061336) c3(McKusick-Kaufman/Bardet-Biedl syndromes putative chaperonin) c4(200134) c5(200135, 200136, 200137, 200138, 200139, 200140, 200141, 200142, 200143, 200144) c6( ) c7(IP, qQ, cev, cz, fd, aj, v, vm, nA, J, anl, qM, Nj, ago, lR, oV, ceu, Tp, bUh, bE, ei, bD, B); #c1(MKL1) c2(NP_001269589) c3(MKL/myocardin-like protein 1 isoform 1) c4( ) c5(102087, 102088, 102089, 102090, 102091, 102092, 102093, 102094) c6( ) c7(T, aHV, fG, if, ht, eX, aA, v, j, ci, hF, akU, dV, aT, h, eV, iY, hu, lZ, aW, aq, aF, t, f, iK, LB, ck, bP, gX, aS, m, bt, B); #c1(MKL1) c2(NP_001269590) c3(MKL/myocardin-like protein 1 isoform 2) c4(102095) c5(102096, 102097, 102098, 102099, 102100) c6( ) c7(T, aHV, fG, if, ht, eX, aA, v, j, ci, hF, akU, dV, aT, h, eV, iY, hu, lZ, aW, aq, aF, t, f, iK, LB, ck, bP, gX, aS, m, bt, B); #c1(MKL1) c2(NP_001269591) c3(MKL/myocardin-like protein 1 isoform 3) c4(102101) c5(102102, 102103, 102104, 102105, 102106, 102107) c6( ) c7(T, aHV, fG, if, ht, ex, aA, v, j, ci, hF, akU, dV, aT, h, eV, iY, hu, lZ, aW, aq, aF, t, f, iK, LB, ck, bP, gX, aS, m, bt, B); #c1(MKL1) c2(XP_005261749) c3(MKL/myocardin-like protein 1 isoform X3) c4(277595) c5(277596) c6( ) c7(T, aHV, fG, if, ht, ex, aA, v, j, ci, hF, akU, dV, aT, h, eV, iY, hu, lZ, aW, aq, aF, t, f, iK, LB, ck, bP, gX, aS, m, bt, B); #c1(MKL1) c2(XP_011528586) c3(MKL/myocardin-like protein 1 isoform X4) c4( ) c5(323806) c6( ) c7(T, aHV, fG, if, ht, eX, aA, v, j, ci, hF, akU, dV, aT, h, eV, iY, hu, lZ, aW, aq, aF, t, f, iK, LB, ck, bP, gX, aS, m, bt, B); #c1(MKL1) c2(XP_011528589) c3(MKL/myocardin-like protein 1 isoform X6) c4( ) c5(323807, 323808, 323809, 323810, 323811, 323812) c6( ) c7(T, aHV, fG, if, ht, eX, aA, v, j, ci, hF, akU, dV, aT, h, eV, iY, hu, lZ, aW, aq, aF, t, f, iK, LB, ck, bP, gX, aS, m, bt, B); #c1(MKL2) c2(NP_054767) c3(MKL/myocardin-like protein 2) c4(177704) c5(177705, 177706, 177707, 177708, 177709, 177710) c6( ) c7(awO, VX, hj, v, aS, QK, bN); #c1(MKL2) c2(XP_005255512) c3(MKL/myocardin-like protein 2 isoform X3) c4(273686, 273687) c5(273688, 273689, 273690, 273691, 273692, 273693, 273694, 273695, 273696, 273697, 273698, 273699) c6( ) c7(awO, VX, hj, v, aS, QK, bN); #c1(MKLN1) c2(NP_037387) c3(muskelin isoform 2) c4(175187) c5(175188, 175189, 175190, 175191, 175192, 175193, 175194) c6( ) c7(Ea); #c1(MKLN1) c2(XP_005250413) c3(muskelin isoform X2) c4(270301) c5(270302, 270303, 270304, 270305, 270306) c6( ) c7(Ea); #c1(MKNK1) c2(XP_011540651) c3(MAP kinase-interacting serine/threonine-protein kinase 1 isoform X7) c4(339199) c5(339200, 339201, 339202, 339203, 339204, 339205) c6( ) c7(eW, T, d, lI, aW, ep, aT, fd, lN, y, lB, eA); #c1(MKNK2) c2(NP_060042) c3(MAP kinase-interacting serine/threonine-protein kinase 2 isoform 1) c4(193847) c5(193848, 193849, 193850, 193851, 193852, 193853) c6( ) c7(kp); #c1(MKNK2) c2(NP_951009) c3(MAP kinase-interacting serine/threonine-protein kinase 2 isoform 2) c4(260965, 260966, 260967) c5(260968, 260969, 260970, 260971, 260972, 260973, 260974, 260975, 260976, 260977, 260978, 260979, 260980, 260981, 260982, 260983, 260984, 260985) c6( ) c7(kp); #c1(MKRN1) c2(NP_001138597) c3(E3 ubiquitin-protein ligase makorin-1 isoform 2) c4(57155) c5(57156, 57157, 57158, 57159, 57160, 57161) c6( ) c7(aF, aA); #c1(MKRN1) c2(NP_038474) c3(E3 ubiquitin-protein ligase makorin-1 isoform 1) c4(176648) c5(176649, 176650, 176651, 176652, 176653, 176654, 176655) c6( ) c7(aF, aA); #c1(MKRN1) c2(XP_011514298) c3(E3 ubiquitin-protein ligase makorin-1 isoform X1) c4(306981) c5( ) c6( ) c7(aF, aA); #c1(MKRN1) c2(XP_011514300) c3(E3 ubiquitin-protein ligase makorin-1 isoform X2) c4(306982) c5(306983, 306984, 306985, 306986, 306987, 306988, 306989, 306990, 306991, 306992, 306993) c6( ) c7(aF, aA); #c1(MKRN3) c2(NP_005655) c3(probable E3 ubiquitin-protein ligase makorin-3) c4(158314) c5(158315, 158316, 158317, 158318, 158319, 158320) c6( ) c7(rc, cex, agl, rk, cew); #c1(MKS1) c2(NP_001159399) c3(Meckel syndrome type I protein isoform 2) c4(63955, 63956) c5(63957, 63958, 63959, 63960, 63961, 63962) c6( ) c7(ago, d, eM, Qu, aW, nX, ff, atS, aT, cey, aiL, vm, zG, Xr, en, Tp, Nj, bz, eA, ix); #c1(MKS1) c2(NP_060247) c3(Meckel syndrome type 1 protein isoform 1) c4(194997, 194998, 194999) c5(195000, 195001, 195002, 195003, 195004, 195005, 195006) c6( ) c7(ago, d, eM, Qu, aW, nX, ff, atS, aT, cey, aiL, vm, zG, Xr, en, Tp, Nj, bz, eA, ix); #c1(MKS1) c2(XP_005257540) c3(Meckel syndrome type 1 protein isoform X3) c4( ) c5(275232, 275233, 275234, 275235, 275236, 275237, 275238, 275239, 275240, 275241, 275242, 275243) c6( ) c7(ago, d, eM, Qu, aW, nX, ff, atS, aT, cey, aiL, vm, zG, Xr, en, Tp, Nj, bz, eA, ix); #c1(MKS1) c2(XP_005257543) c3(Meckel syndrome type 1 protein isoform X8) c4( ) c5(275244, 275245, 275246, 275247, 275248) c6( ) c7(ago, d, eM, Qu, aW, nX, ff, atS, aT, cey, aiL, vm, zG, Xr, en, Tp, Nj, bz, eA, ix); #c1(MKS1) c2(XP_011523261) c3(Meckel syndrome type 1 protein isoform X4) c4(317778) c5(317779, 317780, 317781, 317782, 317783, 317784) c6( ) c7(ago, d, eM, Qu, aW, nX, ff, atS, aT, cey, aiL, vm, zG, Xr, en, Tp, Nj, bz, eA, ix); #c1(MLANA) c2(NP_005502) c3(melanoma antigen recognized by T-cells 1) c4(156858) c5(156859, 156860, 156861, 156862, 156863, 156864, 156865, 156866, 156867, 156868, 156869, 156870, 156871, 156872) c6( ) c7(T, dA, Sv, ci, EA, qj, qN, fr, V, bNX, ds, Xh, cp, aFS, eG, kj, asf, Y, HA, ks, lo, bu, kk, cm, iP, hk, pC); #c1(MLC1) c2(NP_631941) c3(membrane protein MLC1) c4(238370, 238371, 238372, 238373, 238374) c5(238375, 238376, 238377, 238378, 238379, 238380, 238381, 238382, 238383, 238384, 238385, 238386, 238387, 238388, 238389, 238390, 238391, 238392, 238393, 238394, 238395, 238396, 238397, 238398, 238399, 238400, 238401, 238402, 238403, 238404, 238405, 238406, 238407, 238408) c6( ) c7(bkZ, gh, aPn, aW, qK, aT, aNN, cy, dz, cz, bK, ci, as, mk, cb, hF, bt, B, dr, vQ); #c1(MLC1) c2(XP_011528980) c3(membrane protein MLC1 isoform X1) c4(324316) c5(324317, 324318, 324319, 324320, 324321, 324322) c6( ) c7(bkZ, gh, aPn, aW, qK, aT, aNN, cy, dz, cz, bK, ci, as, mk, cb, hF, bt, B, dr, vQ); #c1(MLEC) c2(NP_055545) c3(malectin isoform1 precursor) c4(181882) c5(181883, 181884, 181885, 181886, 181887, 181888) c6( ) c7(cd); #c1(MLF1) c2(NP_001123629) c3(myeloid leukemia factor 1 isoform 2) c4(47881) c5(47882, 47883, 47884, 47885, 47886, 47887, 47888, 47889, 47890, 47891, 47892) c6( ) c7(ck, T, ac, iT, e, aw, bs, aA, af, j, K, jj, bw, bt, eu, br); #c1(MLF1) c2(NP_001182361) c3(myeloid leukemia factor 1 isoform 3) c4(74859, 74860) c5(74861, 74862, 74863, 74864, 74865, 74866, 74867) c6( ) c7(ck, T, ac, iT, e, aw, bs, aA, aF, j, K, jj, bw, bt, eu, br); #c1(MLF1) c2(NP_001182362) c3(myeloid leukemia factor 1 isoform 4) c4(74868) c5(74869, 74870, 74871, 74872, 74873) c6( ) c7(ck, T, ac, iT, e, aw, bs, aA, aF, j, K, jj, bw, bt, eu, br); #c1(MLF1) c2(NP_071888) c3(myeloid leukemia factor 1 isoform 1) c4(212102) c5(212103, 212104, 212105, 212106, 212107, 212108, 212109, 212110, 212111, 212112) c6( ) c7(ck, T, ac, iT, e, aw, bs, aA, aF, j, K, jj, bw, bt, eu, br); #c1(MLF1) c2(XP_005247536) c3(myeloid leukemia factor 1 isoform X3) c4( ) c5(267864) c6( ) c7(ck, T, ac, iT, e, aw, bs, aA, aF, j, K, jj, bw, bt, eu, br); #c1(MLF1) c2(XP_011511154) c3(myeloid leukemia factor 1 isoform X2) c4( ) c5(301582, 301583, 301584, 301585, 301586, 301587, 301588, 301589) c6( ) c7(ck, T, ac, iT, e, aw, bs, aA, aF, j, K, jj, bw, bt, eu, br); #c1(MLF2) c2(NP_005430) c3(myeloid leukemia factor 2) c4(156110) c5(156111, 156112, 156113, 156114, 156115, 156116, 156117, 156118, 156119, 156120, 156121, 156122, 156123, 156124, 156125, 156126, 156127, 156128) c6( ) c7(aW, hF, ad, aT); #c1(MLH1) c2(NP_000240) c3(DNA mismatch repair protein Mlh1 isoform 1) c4(5383, 5384, 5385) c5(5386, 5387, 5388, 5389, 5390, 5391, 5392, 5393, 5394) c6( ) c7(T, os, aC, fR, if, fd, bB, ci, x, afr, aE, K, hu, eL, cp, Qe, aW, LZ, nY, Qq, kt, NR, Ku, g, as, ir, fT, eM, nB, iA, cj, ceA, m, iu, wu, sw, zX, ht, NT, n, fr, qN, eW, BI, ft, hw, Do, pe, kH, h, eV, auS, hn, dL, aDC, HB, KV, aDW, dx, pl, OV, AQ, ge, hQ, aq, fe, kp, cM, Ld, Zj, bP, dB, hk, Qu, hL, at, it, B, fM, cez, eX, ew, aA, hy, lu, q, er, eA, mj, BJ, eO, aT, vH, ae, ne, qw, avg, s, ND, vT, NE, Y, ev, aEk, iU, xR, r, eS, en, bu, cl, t, Px, Vi, iK, zJ, ck, sS, nM, Dy, aDA, iM, au, gX, anD, bt, rA, Mb, id, aD, aJF, jh, KO, oZ, Gn, iJ, ad, dP, av, avp, hd, Db, NS, Yo, io, ji, lQ, wm, YQ, fb, iL, Yj, d, BN, BO, Ub, qt, je, ff, aeV, Ej, aF, hp, iV, iR, adj, bz, dr, dR, bF, es, baa, aoR, Ew, amS, bE, afd, lO, be, adg, hE, ra, X, jr); #c1(MLH1) c2(NP_001161089) c3(DNA mismatch repair protein Mlh1 isoform 2) c4(65306) c5(65307, 65308, 65309, 65310, 65311, 65312, 65313) c6( ) c7(T, os, aC, fR, if, fd, bB, ci, x, afr, aE, K, hu, eL, cp, Qe, aW, LZ, nY, Qq, kt, NR, Ku, g, as, ir, fT, eM, nB, iA, cj, ceA, m, iu, wu, sw, zX, ht, NT, n, fr, qN, eW, BI, ft, hw, Do, pe, kH, h, eV, auS, hn, dL, aDC, hB, KV, aDW, dx, pl, OV, AQ, ge, hQ, aq, fe, kp, cM, Ld, Zj, bP, dB, hk, Qu, hL, at, it, B, fM, cez, eX, ew, aA, hy, lu, q, er, eA, mj, BJ, eO, aT, vH, ae, ne, qw, avg, s, ND, vT, NE, Y, ev, aEk, iU, xR, r, eS, en, bu, cl, t, Px, Vi, iK, zJ, ck, sS, nM, Dy, aDA, iM, au, gX, anD, bt, rA, Mb, id, aD, aJF, jh, KO, oZ, Gn, iJ, ad, dP, av, avp, hd, Db, NS, Yo, io, ji, lQ, wm, YQ, fb, iL, Yj, d, BN, BO, Ub, qt, je, ff, aeV, Ej, aF, hp, iV, iR, adj, bz, dr, dR, bF, es, baa, aoR, Ew, amS, bE, afd, lO, be, adg, hE, ra, X, jr); #c1(MLH1) c2(NP_001245200) c3(DNA mismatch repair protein Mlh1 isoform 4) c4(92643, 92644, 92645, 92646, 92647, 92648) c5(92649, 92650, 92651, 92652, 92653, 92654, 92655, 92656, 92657, 92658, 92659, 92660, 92661, 92662, 92663, 92664, 92665, 92666, 92667, 92668, 92669, 92670, 92671, 92672, 92673, 92674, 92675, 92676, 92677, 92678) c6( ) c7(T, os, aC, fR, if, fd, bB, ci, x, afr, aE, K, hu, eL, cp, Qe, aW, LZ, nY, Qq, kt, NR, Ku, g, as, ir, fT, eM, nB, iA, cj, ceA, m, iu, wu, sw, zX, ht, NT, n, fr, qN, eW, BI, ft, hw, Do, pe, kH, h, eV, auS, hn, dL, aDC, hB, KV, aDW, dx, pl, OV, AQ, ge, hQ, aq, fe, kp, cM, Ld, Zj, bP, dB, hk, Qu, hL, at, it, B, fM, cez, eX, ew, aA, hy, lu, q, er, eA, mj, BJ, eO, aT, vH, ae, ne, qw, avg, s, ND, vT, NE, Y, ev, aEk, iU, xR, r, eS, en, bu, cl, t, Px, Vi, iK, zJ, ck, sS, nM, Dy, aDA, iM, au, gX, anD, bt, rA, Mb, id, aD, aJF, jh, KO, oZ, Gn, iJ, ad, dP, av, avp, hd, Db, NS, Yo, io, ji, lQ, wm, YQ, fb, iL, Yj, d, BN, BO, Ub, qt, je, ff, aeV, Ej, aF, hp, iV, iR, adj, bz, dr, dR, bF, es, baa, aoR, Ew, amS, bE, afd, lO, be, adg, hE, ra, X, jr); #c1(MLH1) c2(NP_001245202) c3(DNA mismatch repair protein Mlh1 isoform 3) c4( ) c5(92679, 92680, 92681, 92682, 92683, 92684, 92685, 92686, 92687, 92688, 92689, 92690, 92691, 92692, 92693, 92694) c6( ) c7(T, os, aC, fR, if, fd, bB, ci, x, afr, aE, K, hu, eL, cp, Qe, aW, LZ, nY, Qq, kt, NR, Ku, g, as, ir, fT, eM, nB, iA, cj, ceA, m, iu, wu, sw, zX, ht, NT, n, fr, qN, eW, BI, ft, hw, Do, pe, kH, h, eV, auS, hn, dL, aDC, hB, KV, aDW, dx, pI, OV, AQ, ge, hQ, aq, fe, kp, cM, Ld, Zj, bP, dB, hk, Qu, hL, at, it, B, fM, cez, eX, ew, aA, hy, lu, q, er, eA, mj, BJ, eO, aT, vH, ae, ne, qw, avg, s, ND, vT, NE, Y, ev, aEk, iU, xR, r, eS, en, bu, cl, t, Px, Vi, iK, zJ, ck, sS, nM, Dy, aDA, iM, au, gX, anD, bt, rA, Mb, id, aD, aJF, jh, KO, oZ, Gn, iJ, ad, dP, av, avp, hd, Db, NS, Yo, io, ji, lQ, wm, YQ, fb, iL, Yj, d, BN, BO, Ub, qt, je, ff, aeV, Ej, aF, hp, iV, iR, adj, bz, dr, dR, bF, es, baa, aoR, Ew, amS, bE, afd, lO, be, adg, hE, ra, X, jr); #c1(MLH1) c2(XP_005265218) c3(DNA mismatch repair protein Mlh1 isoform X2) c4( ) c5(279449) c6( ) c7(T, os, aC, fR, if, fd, bB, ci, x, afr, aE, K, hu, eL, cp, Qe, aW, LZ, nY, Qq, kt, NR, Ku, g, as, ir, fT, eM, nB, iA, cj, ceA, m, iu, wu, sw, zX, ht, NT, n, fr, qN, eW, BI, ft, hw, Do, pe, kH, h, eV, auS, hn, dL, aDC, hB, KV, aDW, dx, pl, OV, AQ, ge, hQ, aq, fe, kp, cM, Ld, Zj, bP, dB, hk, Qu, hL, at, it, B, fM, cez, eX, ew, aA, hy, lu, q, er, eA, mj, BJ, eO, aT, vH, ae, ne, qw, avg, s, ND, vT, NE, Y, ev, aEk, iU, xR, r, eS, en, bu, cl, t, Px, Vi, iK, zJ, ck, sS, nM, Dy, aDA, iM, au, gX, anD, bt, rA, Mb, id, aD, aJF, jh, KO, oZ, Gn, iJ, ad, dP, av, avp, hd, Db, NS, Yo, io, ji, lQ, wm, YQ, fb, iL, Yj, d, BN, BO, Ub, qt, je, ff, aeV, Ej, aF, hp, iV, iR, adj, bz, dr, dR, bF, es, baa, aoR, Ew, amS, bE, afd, lO, be, adg, hE, ra, X, jr); #c1(MLH1) c2(XP_011532029) c3(DNA mismatch repair protein Mlh1 isoform X4) c4( ) c5(328092, 328093, 328094, 328095, 328096) c6( ) c7(T, os, aC, fR, if, fd, bB, ci, x, afr, aE, K, hu, eL, cp, Qe, aW, LZ, nY, Qq, kt, NR, Ku, g, as, ir, fT, eM, nB, iA, cj, ceA, m, iu, wu, sw, zX, ht, NT, n, fr, qN, eW, BI, ft, hw, Do, pe, kH, h, eV, auS, hn, dL, aDC, hB, KV, aDW, dx, pl, OV, AQ, ge, hQ, aq, fe, kp, cM, Ld, Zj, bP, dB, hk, Qu, hL, at, it, B, fM, cez, eX, ew, aA, hy, lu, q, er, eA, mj, BJ, eO, aT, vH, ae, ne, qw, avg, s, ND, vT, NE, Y, ev, aEk, iU, xR, r, eS, en, bu, cl, t, Px, Vi, iK, zJ, ck, sS, nM, Dy, aDA, iM, au, gX, anD, bt, rA, Mb, id, aD, aJF, jh, KO, oZ, Gn, iJ, ad, dP, av, avp, hd, Db, NS, Yo, io, ji, lQ, wm, YQ, fb, iL, Yj, d, BN, BO, Ub, qt, je, ff, aeV, Ej, aF, hp, iV, iR, adj, bz, dr, dR, bF, es, baa, aoR, Ew, amS, bE, afd, lO, be, adg, hE, ra, X, jr); #c1(MLH3) c2(NP_055196) c3(DNA mismatch repair protein Mlh3 isoform 2) c4(179711) c5(179712, 179713, 179714, 179715, 179716, 179717) c6( ) c7(T, gU, BO, ND, bE, aW, ht, gX, aT, r, bB, hL, dL, vH, m, ceB, cl, t, q); #c1(MLH3) c2(XP_005267588) c3(DNA mismatch repair protein Mlh3 isoform X1) c4(281055, 281056, 281057) c5(281058, 281059, 281060, 281061, 281062, 281063, 281064, 281065, 281066, 281067, 281068, 281069, 281070, 281071, 281072, 281073) c6( ) c7(T, gU, BO, ND, bE, aW, ht, gX, aT, r, bB, hL, dL, vH, m, ceB, cl, t, q); #c1(MLH3) c2(XP_005267589) c3(DNA mismatch repair protein Mlh3 isoform X2) c4(281074) c5(281075, 281076, 281077, 281078, 281079, 281080) c6( ) c7(T, gU, BO, ND, bE, aW, ht, gX, aT, r, bB, hL, dL, vH, m, ceB, cl, t, q); #c1(MLH3) c2(XP_011534949) c3(DNA mismatch repair protein Mlh3 isoform X5) c4(331512) c5( ) c6( ) c7(T, gU, BO, ND, bE, aW, ht, gX, aT, r, bB, hL, dL, vH, m, ceB, cl, t, q); #c1(MLH3) c2(XP_011534950) c3(DNA mismatch repair protein Mlh3 isoform X7) c4( ) c5(331513, 331514, 331515, 331516, 331517, 331518, 331519, 331520, 331521, 331522, 331523, 331524, 331525) c6( ) c7(T, gU, BO, ND, bE, aW, ht, gX, aT, r, bB, hL, dL, vH, m, ceB, cl, t, q); #c1(MLKL) c2(NP_001135969) c3(mixed lineage kinase domain-like protein isoform 2) c4(53767) c5(53768, 53769, 53770, 53771, 53772, 53773) c6( ) c7(fe, ceC); #c1(MLKL) c2(NP_689862) c3(mixed lineage kinase domain-like protein isoform 1) c4(244195) c5(244196, 244197, 244198, 244199, 244200, 244201) c6( ) c7(fe, ceC); #c1(MLLT1) c2(NP_005925) c3(protein ENL) c4(160478) c5(160479, 160480, 160481, 160482, 160483, 160484, 160485) c6( ) c7(ck, x, eG, iT, Bj, k, jj, hF, bt, kS, abh); #c1(MLLT10) c2(NP_001182555) c3(protein AF-10 isoform c) c4(75039) c5(75040, 75041, 75042, 75043, 75044, 75045) c6( ) c7(ck, x, T, iT, iZ, Y, hF, r, fd, dz, iY, fr, k, Fu, hf, ajA, bt, iJ, q, ip); #c1(MLLT10) c2(NP_001182556) c3(protein AF-10 isoform d) c4(75046) c5(75047, 75048, 75049, 75050, 75051, 75052, 75053, 75054, 75055, 75056, 75057, 75058) c6( ) c7(ck, x, T, lT, iZ, Y, hF, r, fd, dz, iY, fr, k, Fu, hf, ajA, bt, iJ, q, ip); #c1(MLLT10) c2(NP_001182557) c3(protein AF-10 isoform e) c4(75059) c5(75060, 75061, 75062, 75063, 75064, 75065) c6( ) c7(ck, x, T, iT, iZ, Y, hF, r, fd, dz, iY, fr, k, Fu, hf, ajA, bt, iJ, q, ip); #c1(MLLT10) c2(NP_004632) c3(protein AF-10 isoform a) c4(148351, 148352, 148353) c5(148354, 148355, 148356, 148357, 148358, 148359, 148360, 148361, 148362, 148363, 148364, 148365, 148366, 148367, 148368, 148369, 148370, 148371, 148372, 148373, 148374, 148375, 148376, 148377, 148378, 148379, 148380, 148381) c6( ) c7(ck, x, T, iT, iZ, Y, hF, r, fd, dz, iY, fr, k, Fu, hf, ajA, bt, iJ, q, ip); #c1(MLLT10) c2(XP_005252662) c3(protein AF-10 isoform X4) c4(271976, 271977) c5(271978) c6( ) c7(ck, x, T, iT, iZ, Y, hF, r, fd, dz, iY, fr, k, Fu, hf, ajA, bt, iJ, q, ip); #c1(MLLT10) c2(XP_005252666) c3(protein AF-10 isoform X7) c4(271979, 271980) c5(271981, 271982, 271983, 271984, 271985, 271986, 271987, 271988, 271989, 271990, 271991, 271992) c6( ) c7(ck, x, T, iT, iZ, Y, hF, r, fd, dz, iY, fr, k, Fu, hf, ajA, bt, iJ, q, ip); #c1(MLLT3) c2(NP_001273620) c3 (protein AF-9 isoform b) c4(105459, 105460) c5(105461, 105462, 105463, 105464, 105465, 105466, 105467, 105468, 105469, 105470, 105471, 105472, 105473, 105474, 105475, 105476, 105477, 105478, 105479, 105480, 105481, 105482, 105483, 105484, 105485, 105486, 105487) c6( ) c7(ck, x, ceD, T, iZ, ha, bK, k, bt, af); #c1(MLLT3) c2(NP_004520) c3(protein AF-9 isoform a) c4(147197, 147198, 147199) c5(147200, 147201, 147202, 147203, 147204, 147205, 147206, 147207) c6( ) c7(ck, x, ceD, T, iZ, ha, bK, k, bt, af); #c1(MLLT4) c2(NP_001035089) c3(afadin isoform 2) c4(32484) c5(32485, 32486, 32487, 32488, 32489, 32490) c6( ) c7(T, aW, aT, m, bt, eW); #c1(MLLT4) c2(NP_001193937) c3(afadin isoform 1) c4(83243) c5(83244, 83245, 83246, 83247, 83248, 83249) c6( ) c7(T, aW, aT, m, bt, eW); #c1(MLLT4) c2(XP_005267053) c3(afadin isoform X1) c4(280644, 280645, 280646, 280647) c5(280648, 280649, 280650, 280651, 280652, 280653, 280654, 280655, 280656, 280657, 280658, 280659, 280660) c6( ) c7(T, aW, aT, m, bt, eW); #c1(MLLT4) c2(XP_006715555) c3(afadin isoform X9) c4( ) c5(289604, 289605, 289606, 289607, 289608, 289609, 289610, 289611, 289612, 289613, 289614) c6( ) c7(T, aW, aT, m, bt, eW); #c1(MLLT4) c2(XP_006715557) c3(afadin isoform X8) c4( ) c5(289615) c6( ) c7(T, aW, aT, m, bt, eW); #c1(MLLT6) c2(NP_005928) c3(protein AF-17) c4(160494) c5( ) c6( ) c7(ck, hF, on, k, bt); #c1(MLLT6) c2(XP_005257436) c3(protein AF-17 isoform X5) c4( ) c5(275113) c6( ) c7(ck, hF, on, k, bt); #c1(MLLT6) c2(XP_006721974) c3(protein AF-17 isoform X2) c4(294511) c5(294512, 294513, 294514, 294515, 294516, 294517) c6( ) c7(ck, hF, on, k, bt); #c1(MLLT6) c2(XP_011523121) c3(protein AF-17 isoform X6) c4( ) c5(317630, 317631, 317632, 317633, 317634, 317635, 317636) c6( ) c7(ck, hF, on, k, bt); #c1(MLN) c2(NP_001035198) c3(promotilin isoform 2 preproprotein) c4(32589) c5(32590, 32591, 32592, 32593, 32594, 32595, 32596, 32597) c6( ) c7(xS, aaT, Pf, aW, fS, aZL, eZ, aT, hD, fd, Ja, hp, K, v, iV, aeP, af, MG); #c1(MLN) c2(NP_001171627) c3(promotilin isoform 3 preproprotein) c4(71017) c5(71018, 71019, 71020, 71021, 71022, 71023, 71024, 71025) c6( ) c7(xS, aaT, Pf, aW, fS, aZL, eZ, aT, hD, fd, Ja, hp, K, v, iV, aeP, af, MG); #c1(MLN) c2(NP_002409) c3(promotilin isoform 1 preproprotein) c4(128177) c5(128178, 128179, 128180, 128181, 128182, 128183, 128184, 128185) c6( ) c7(xS, aaT, Pf, aW, fS, aZL, eZ, aT, hD, fd, Ja, hp, K, v, iV, aeP, af, MG); #c1(MLNR) c2(NP_001498) c3(motilin receptor) c4(118200, 118201) c5(118202, 118203, 118204, 118205, 118206, 118207, 118208, 118209, 118210, 118211, 118212, 118213) c6( ) c7(FE, QK); #c1(MLPH) c2(NP_001035932) c3(melanophilin isoform 2) c4(33463, 33464) c5(33465, 33466, 33467, 33468, 33469, 33470) c6( ) c7(ceE, ad, bN, ip); #c1(MLPH) c2(NP_001268403) c3(melanophilin isoform 4) c4( ) c5(99840, 99841, 99842, 99843, 99844, 99845, 99846, 99847, 99848, 99849, 99850) c6( ) c7(ceE, ad, bN, ip); #c1(MLPH) c2(NP_077006) c3(melanophilin isoform 1) c4(214701, 214702) c5(214703, 214704, 214705, 214706, 214707, 214708, 214709, 214710, 214711, 214712, 214713, 214714) c6( ) c7(ceE, ad, bN, ip); #c1(MLPH) c2(XP_006712803) c3(melanophilin isoform X4) c4(286843) c5( ) c6( ) c7(ceE, ad, bN, ip); #c1(MLST8) c2(NP_001186102) c3(target of rapamycin complex subunit LST8 isoform a) c4(77130, 77131) c5(77132, 77133, 77134, 77135, 77136, 77137, 77138, 77139, 77140, 77141, 77142, 77143, 77144, 77145, 77146, 77147, 77148, 77149, 77150) c6( ) c7(it, T, aF, adz, aA); #c1(MLST8) c2(XP_005255532) c3(target of rapamycin complex subunit LST8 isoform X1) c4(273719) c5(273720, 273721, 273722, 273723, 273724, 273725, 273726, 273727, 273728, 273729, 273730) c6( ) c7(it, T, aF, adz, aA); #c1(MLX) c2(NP_733752) c3(max-like protein X isoform gamma) c4(246948) c5(246949, 246950, 246951, 246952, 246953, 246954) c6( ) c7(v, af); #c1(MLX) c2(NP_937847) c3(max-like protein X isoform beta) c4(259279) c5(259280, 259281, 259282, 259283, 259284, 259285, 259286) c6( ) c7(v, af); #c1(MLX) c2(NP_937848) c3(max-like protein X isoform alpha) c4(259287) c5(259288, 259289, 259290, 259291, 259292, 259293) c6( ) c7(v, af); #c1(MLX1P) c2(NP_055753) c3(MLX-interacting protein) c4(183292, 183293, 183294) c5(183295, 183296, 183297, 183298, 183299, 183300, 183301, 183302, 183303, 183304, 183305, 183306, 183307) c6( ) c7(bL, x, T, aE, bE, Y, cj, bB, fr, k, iK, B); #c1(MLX1P) c2(XP_006719357) c3(MLX-interacting protein isoform X5) c4( ) c5(292433, 292434, 292435, 292436, 292437, 292438) c6( ) c7(bL, x, T, aE, bE, Y, cj, bB, fr, k, iK, B); #c1(MLX1PL) c2(NP_116569) c3(carbohydrate-responsive element-binding protein isoform alpha) c4(226229, 226230) c5(226231, 226232, 226233, 226234, 226235, 226236, 226237, 226238, 226239, 226240, 226241, 226242) c6( ) c7(bL, T, eR, e, aj, dG, j, v, q, J, aT, ae, af, eu, qt, aW, Bw, fc, r, Mz, as, aIC, ac, cO, mf, cP, ei, B); #c1(MLX1PL) c2(NP_116570) c3(carbohydrate-responsive element-binding protein isoform beta) c4(226243) c5(226244, 226245, 226246, 226247, 226248, 226249) c6( ) c7(bL, T, eR, e, aj, dG, j, v, q, J, aT, ae, af, eu, qt, aW, Bw, fc, r, Mz, as, aIC, ac, cO, mf, cP, ei, B); #c1(MLX1PL) c2(NP_116571) c3(carbohydrate-responsive element-binding protein isoform gamma) c4(226250) c5(226251, 226252, 226253, 226254, 226255, 226256) c6( ) c7(bL, T, eR, e, aj, dG, j, v, q, J, aT, ae, af, eu, qt, aW, Bw, fc, r, Mz, as, aIC, ac, cO, mf, cP, ei, B); #c1(MLX1PL) c2(NP_116572) c3(carbohydrate-responsive element-binding protein isoform delta) c4(226257) c5(226258, 226259, 226260, 226261, 226262, 226263) c6( ) c7(bL, T, eR, e, aj, dG, j, v, q, J, aT, ae, af, eu, qt, aW, Bw, fc, r, Mz, as, aIC, ac, cO, mf, cP, ei, B); #c1(MLYCD) c2(NP_036345) c3(malonyl-CoA decarboxylase, mitochondrial) c4(173104) c5(173105, 173106, 173107, 173108, 173109, 173110) c6( ) c7(mO, cc, vN, uL, oV, ha, lC, bKw, zN, zO, vm, dy, uW, afx, af); #c1(MMAA) c2(NP_758454) c3(methylmalonic aciduria type A protein, mitochondrial precursor) c4(249208, 249209, 249210) c5(249211, 249212, 249213, 249214, 249215, 249216, 249217, 249218, 249219, 249220, 249221, 249222, 249223) c6( ) c7(ceF, ceH, ceI, ao, ceG); #c1(MMAA) c2(XP_011529988) c3(methylmalonic aciduria type A protein, mitochondrial isoform X2) c4( ) c5(325800, 325801, 325802, 325803, 325804) c6( ) c7(ceF, ceH, ceI, ao, ceG); #c1(MMAB) c2(NP_443077) c3(cob(l)yrinic acid a,c-diamide adenosyltransferase, mitochondrial precursor) c4(229284) c5(229285, 229286, 229287, 229288, 229289, 229290) c6( ) c7(bL, aHq, T, os, dD, aC, id, ea, WL, aw, ew, aA, jh, aj, oZ, j, ct, nA, eq, WC, V, J, x, ceG, hb, aT, h, ey, K, dQ, WF, uQ, sb, A, d, kj, wr, aW, WE, Y, ff, nf, kp, aF, en, t, cm, dr, WH, qe, cO, cj, Km, k, m, ER, WI, bC, eo); #c1(MMAB) c2(XP_011536570) c3(cob(l)yrinic acid a,c-diamide adenosyltransferase, mitochondrial isoform X2) c4(334078) c5(334079, 334080, 334081, 334082, 334083, 334084) c6( ) c7(bL, aHq, T, os, dD, aC, id, ea, WL, aw, ew, aA, jh, aj, oZ, j, ct, nA, eq, WC, V, J, x, ceG, hb, aT, h, ey, K, dQ, WF, uQ, sb, A, d, kj, wr, aW, WE, Y, ff, nf, kp, aF, en, t, cm, dr, WH, qe, cO, cj, Km, k, m, ER, WI, bC, eo); #c1(MMADHC) c2(NP_056517) c3(methylmalonic aciduria and homocystinuria type D protein, mitochondrial) c4(187899) c5(187900, 187901, 187902, 187903, 187904, 187905, 187906, 187907, 187908, 187909) c6( ) c7(MX, ceL, ceK, gv, ad, ceJ); #c1(MMD) c2(NP_036461) c3(monocyte to macrophage differentiation factor) c4(174040) c5(174041, 174042, 174043, 174044, 174045, 174046) c6( ) c7(T, id, ac, eM, Qu, aJ, xs, EQ, cz, j, aS, Ea, bd, ey, fq); #c1(MMD2) c2(NP_001094070) c3(monocyte to macrophage differentiation factor 2 isoform 1) c4(40610) c5(40611, 40612, 40613, 40614, 40615, 40616) c6( ) c7(ceM); #c1(MMD2) c2(NP_001257304) c3(monocyte to macrophage differentiation factor 2 isoform 3) c4(95108) c5(95109, 95110, 95111, 95112, 95113) c6( ) c7(ceM); #c1(MMD2) c2(NP_940685) c3(monocyte to macrophage differentiation factor 2 isoform 2) c4(259850) c5(259851, 259852, 259853, 259854, 259855, 259856, 259857) c6( ) c7(ceM); #c1(MME) c2(XP_011511160) c3(neprilysin isoform X1) c4(301590, 301591, 301592, 301593, 301594, 301595) c5(301596, 301597, 301598, 301599, 301600, 301601, 301602, 301603, 301604, 301605, 301606, 301607, 301608, 301609, 301610, 301611, 301612, 301613, 301614, 301615, 301616, 301617, 301618, 301619, 301620, 301621, 301622, 301623, 301624, 301625, 301626, 301627, 301628, 301629, 301630, 301631, 301632, 301633, 301634, 301635, 301636, 301637, 301638, 301639, 301640, 301641, 301642, 301643, 301644, 301645, 301646) c6( ) c7(gh, T, aC, e, l, fd, bB, aeu, ci, ceN, x, aE, oG, aoL, K, H, pk, aW, Qm, g, hJ, cm, bw, ou, bXI, eM, iA, acw, vA, mx, vy, cj, k, R, ajJ, Th, dD, n, fr, cd, ke, b, jz, Bj, h, hn, dL, ox, db, A, kj, Ni, fQ, kp, bXK, ceO, alj, op, hq, hL, it, B, vQ, bL, L, fM, dA, Lx, cz, v, j, q, eA, RI, uT, aT, ae, af, qw, s, iU, Y, aEk, lo, cl, t, iK, Cv, ck, tj, fP, bt, ao, iW, jh, eq, hF, ad, fX, Ae, iL, BN, si, aEm, bz, es, bE, ei, i, xg, Ex); #c1(MMEL1) c2(XP_011540425) c3(membrane metallo-endopeptidase-like 1 isoform X1) c4( ) c5(338919, 338920, 338921) c6( ) c7(T, di, agT, sO, dJ, cL, aj, hp, bc, y, V, ae, af, J, ad); #c1(MMEL1) c2(XP_011544704) c3(membrane metallo-endopeptidase-like 1 isoform X6) c4(343763) c5( ) c6( ) c7(T, di, agT, sO, dJ, cL, aj, hp, bc, y, V, ae, af, J, ad); #c1(MMEL1) c2(XP_011544709) c3(membrane metallo-endopeptidase-like 1 isoform X10) c4(343764) c5(343765, 343766, 343767, 343768, 343769, 343770, 343771, 343772, 343773, 343774, 343775, 343776, 343777, 343778, 343779, 343780) c6(343781, 343782, 343783, 343784) c7(T, di, agT, sO, dJ, cL, aj, hp, bc, y, V, ae, af, J, ad); #c1(MMEL1) c2(XP_011544712) c3(membrane metallo-endopeptidase-like 1 isoform X13) c4(343785, 343786) c5(343787, 343788, 343789, 343790, 343791, 343792, 343793, 343794, 343795, 343796, 343797, 343798, 343799, 343800, 343801, 343802) c6(343803, 343804, 343805, 343806) c7(T, di, agT, sO, dJ, cL, aj, hp, bc, y, V, ae, af, J, ad); #c1(MMP1) c2(NP_002412) c3(interstitial collagenase isoform 1 preproprotein) c4(128225) c5(128226, 128227, 128228, 128229, 128230, 128231) c6( ) c7(bM, T, fR, lP, pf, ea, if, e, aRm, al, eb, bB, aTy, fd, lR, ci, bh, fw, sD, Tz, Hr, dY, aE, Bc, ja, K, bah, hu, ass, eL, cp, aa, hm, eG, yo, aTq, dH, Jl, aW, aEl, az, hl, wf, CT, hN, vs, g, as, pL, eF, uC, aAW, ba, qJ, eM, iA, dc, ceR, vA, Gs, mx, bhl, uo, m, in, ik, iu, cE, Vj, dD, pG, sQ, wJ, sw, zt, aw, ht, n, aj, fr, qN, aBP, cd, ai, eh, jz, no, cc, ec, zw, h, aNj, dL, ey, pj, dx, bzr, Nr, asL, ks, fQ, aq, po, kp, fe, dw, sE, fq, op, kT, GG, W, ky, vD, dB, anC, PI, lf, Be, wk, at, uz, ut, bm, qF, it, B, AN, bL, AS, dA, eR, fG, oe, yk, jx, aA, lj, ceP, cz, hy, j, v, C, bcE, bgR, V, q, J, eA, eJ, bke, RI, lg, tL, ceS, aT, Fx, bq, an, lM, ae, af, dL, bA, kl, aGP, bf, s, oV, Y, ev, fO, Pg, bg, r, aYp, afq, en, cl, bQ, t, Px, iK, pb, asw, Dy, c, ac, ig, lV, gX, uS, cS, np, fD, VB, avi, vi, ao, sY, kh, am, aFe, id, aZ, iW, jh, vr, ah, eq, XM, ad, wD, hX, dP, ceQ, av, hd, F, od, py, yt, Dq, le, bgr, lQ, WZ, uX, TC, d, lU, BO, ff, xs, eH, Ej, bpA, aF, hp, iV, px, bz, DF, Nq, LB, gk, Bv, es, bMX, TA, bE, aka, aFb, aef, aS, Hg, Lz, ei, lC, ne, y); #c1(MMP10) c2(NP_002416) c3(stromelysin-2 preproprotein) c4(128253) c5(128254, 128255, 128256, 128257, 128258, 128259) c6( ) c7(bM, sY, T, yk, aw, al, jh, aA, n, j, ci, tK, ai, av, afr, F, rw, M, dL, ji, ZN, hm, oS, A, lR, bf, ff, xs, hl, ks, Ej, kp, aF, en, iV, t, uC, bz, ac, ky, eM, hk, vy, bm, dy, m, y, iu); #c1(MMP11) c2(NP_005931) c3(stromelysin-3 preproprotein) c4(160503) c5(160504, 160505, 160506, 160507, 160508, 160509) c6( ) c7(sY, T, dD, AS, ea, al, NT, aA, n, Gn, aei, ci, eq, AO, mC, q, ip, BI, F, aE, ZZ, aT, aaj, dL, AN, apj, Dz, in, pj, eL, dx, pl, aa, yi, iU, Jl, aW, qt, ff, eZ, r, Pp, kp, aF, en, YF, cl, uC, aYt, aoO, sS, bP, eM, nB, aGo, hk, hc, Be, aJQ, fL, lf, rA, it, B, hv); #c1(MMP12) c2(NP_002417) c3(macrophage metalloelastase preproprotein) c4(128260) c5(128261, 128262, 128263, 128264, 128265, 128266) c6( ) c7(bM, T, lP, pf, ea, e, al, fo, ci, Qp, aE, lQ, eL, Zm, aiJ, dH, aW, vs, g, hJ, eF, ahv, uC, y, eM, avZ, dD, aw, ht, n, aBP, cd, ai, nm, dL, ey, apc, A, fe, aq, bld, bxB, hq, bm, at, uz, B, fl, eR, fG, oe, yk, aA, cz, j, V, q, eA, yg, aT, qk, qw, bf, r, en, t, pb, ajl, ac, c, ig, lV, np, sY, kh, am, eq, ad, hX, dP, av, F, od, aMn, vB, hP, lQ, uQ, d, lR, Di, TY, ff, aF, bz, TA, aS, ME, ei, lf, X, Ex); #c1(MMP13) c2(NP_002418) c3(collagenase 3 preproprotein) c4(128267, 128268, 128269) c5(128270, 128271, 128272, 128273, 128274, 128275, 128276, 128277, 128278, 128279, 128280, 128281, 128282, 128283, 128284, 128285, 128286, 128287, 128288, 128289, 128290, 128291, 128292, 128293) c6(128294, 128295, 128296, 128297, 128298, 128299) c7(T, ea, al, fd, bB, aTy, zh, ci, Na, afr, bPx, rw, aeO, K, hu, eL, bc, cp, eG, aeR, aW, Az, hl, g, pL, eF, ahv, uC, nB, m, ceV, dD, zt, aw, ceU, n, fr, bd, cd, ai, pe, amJ, rl, dL, Dz, fQ, aq, fe, mo, W, hL, bm, at, pg, it, B, dA, fG, aA, j, C, q, eA, XX, aT, Nc, rE, ae, eu, bA, aGP, bf, oV, Y, fO, Pg, bg, r, en, t, sS, ru, ceT, JH, hS, sY, kh, id, aFe, eq, ad, hX, dP, F, od, hP, ji, ff, eH, aF, bz, LB, zr, bE, aFb, aS, lf, X, y); #c1(MMP14) c2(NP_004986) c3(matrix metalloproteinase-14 preproprotein) c4(151710) c5(151711, 151712, 151713, 151714, 151715, 151716) c6( ) c7(DS, T, aC, lP, pf, ea, if, al, Xi, eb, bB, aTy, ci, aE, rw, K, hu, dQ, H, eL, xQ, eG, aW, g, as, pL, eF, uC, cm, bw, bSA, eM, nB, ki, mx, U, tV, cj, m, uL, iu, dD, sw, aw, ht, n, aj, fr, qN, cd, ai, cc, adL, By, dL, aaA, ey, vx, Gc, pl, A, kp, yq, W, dB, hk, hL, bm, at, pg, it, B, AN, ceW, bL, L, ka, vQ, AS, dA, eR, yk, lH, jx, aA, v, j, V, q, J, eA, aT, cy, ceX, ae, eu, bf, iU, nO, iZ, Y, r, en, t, btO, iK, pb, tR, sk, ac, c, np, dy, im, bt, rA, VB, yv, hS, sY, kh, id, KO, eq, ceY, ad, wD, hX, dP, av, hd, F, ib, py, vw, aQ, nJ, d, ff, eH, aP, af, bz, gk, bE, ll, aS, ME, hE, X, y); #c1(MMP15) c2(NP_002419) c3(matrix metalloproteinase-15 preproprotein) c4(128300) c5(128301, 128302, 128303, 128304, 128305, 128306) c6( ) c7(gx, sY, T, if, al, KO, v, ci, qN, cd, q, eA, wD, iu, F, aE, qK, lG, K, jf, d, r, as, uC, btO, eM, y, bt, B); #c1(MMP16) c2(NP_005932) c3(matrix metalloproteinase-16 precursor) c4(160510) c5(160511, 160512, 160513, 160514, 160515, 160516) c6( ) c7(sY, al, aA, fr, ci, C, qN, lu, eA, ad, Yn, F, sl, ae, af, d, aF, PQ, aXL, as, uC, fq, vK, uJ, y, B); #c1(MMP17) c2(NP_057239) c3(matrix metalloproteinase-17 preproprotein) c4(189655, 189656) c5(189657, 189658, 189659, 189660, 189661, 189662, 189663, 189664, 189665, 189666, 189667, 189668, 189669) c6( ) c7(sY, T, F, aW, al, aT, ci, g, im, y, uC, ai); #c1(MMP17) c2(XP_011536659) c3(matrix metalloproteinase-17 isoform X1) c4(334156) c5(334157, 334158, 334159, 334160, 334161) c6( ) c7(sY, T, F, aW, al, aT, ci, g, im, y, uC, ai); #c1(MMP19) c2(NP_001259030) c3(matrix metalloproteinase-19 isoform 3 preproprotein) c4(96958) c5(96959, 96960, 96961, 96962, 96963, 96964, 96965, 96966) c6(96967, 96968) c7(LB, bM, sY, T, afr, hk, fG, yk, bsO, ff, cr, ci, K, en, g, y, uC, af); #c1(MMP19) c2(NP_002420) c3(matrix metalloproteinase-19 isoform 1 preproprotein) c4(128307, 128308) c5(128309, 128310, 128311, 128312, 128313, 128314, 128315, 128316) c6(128317, 128318) c7(LB, bM, sY, T, afr, hk, fG, yk, bsO, ff, cr, ci, K, en, g, y, uC, af); #c1(MMP19) c2(XP_006719464) c3 (matrix metalloproteinase-19 isoform X3) c4( ) c5(292494, 292495, 292496, 292497, 292498, 292499, 292500, 292501) c6(292502, 292503) c7(LB, bM, sY, T, afr, hk, fG, yk, bsO, ff, cr, ci, K, en, g, y, uC, af); #c1(MMP2) c2(NP_001121363) c3(72 kDa type IV collagenase isoform 2) c4( ) c5(45766, 45767) c6( ) c7(T, ea, fd, fo, aoj, ci, aAo, aY, Hr, bv, aE, fm, rw, hu, cp, yo, Az, uY, vs, ba, fT, iA, nB, wE, k, lS, vF, aFG, aO, ht, aj, cd, Ak, aEL, EU, fQ, fe, ajz, dB, bOj, Be, ZR, apE, jv, jp, DD, aA, cz, AO, q, bIN, qK, cy, dl, eu, bA, aGP, bf, bg, uU, cl, cfa, pb, cT, rR, iQ, ac, bt, ao, cfe, gV, iW, jh, aHv, fu, ceY, ad, wD, hX, yt, le, lQ, WZ, ff, aeV, iV, bz, lf, lC, iu, bM, xS, if, e, bB, Tz, ip, Na, vV, ja, bc, eG, dH, JI, hl, as, eF, cm, qJ, Kv, ki, vA, vy, wx, in, Vj, rt, dD, n, md, brY, jz, zw, ec, WB, eV, aYF, hi, hQ, yq, sE, YE, abV, PI, hL, at, ut, B, vQ, AS, mr, wL, eX, v, V, hW, nQ, ae, jf, iZ, De, Qr, t, Px, ck, bLr, kP, au, gX, JH, zu, avi, vi, hl, ob, Dd, wV, eq, hd, py, ab, vc, nJ, d, hG, NH, dN, dr, aef, y, hv, aC, fR, azf, lP, al, Vp, u, Bc, K, eL, adT, mQ, wf, hN, g, pL, bwe, uC, aAW, aDs, Gs, uo, Hb, m, QO, cfg, sQ, aw, NT, qy, zP, ft, Lv, By, jl, CN, bMq, ks, cM, Ld, W, JM, Pr, it, fl, L, ceW, fM, fG, j, eA, J, XX, amg, cfd, yg, Nc, bq, kl, xR, Ue, Y, r, en, wA, iK, brZ, hc, dh, dy, Mj, gx, sY, kh, id, hF, iJ, dP, oj, io, hP, adx, WW, uX, lU, lR, xs, eH, cr, Fq, iR, cfF, Bv, sG, ei, hE, tQ, pf, wH, zh, dY, cfc, H, hm, DA, aW, hJ, eM, nw, Xu, vl, Mi, fr, bd, qN, KO, ai, ke, BI, cc, rl, h, dL, aUm, hB, vx, wC, bgf, aq, kp, op, Qy, Of, hk, bm, hR, uz, te, ceZ, bL, ka, eR, uv, tc, C, uT, aT, af, s, iU, oV, aYp, ajl, nM, pZ, yW, AH, fP, dT, aDj, hS, vr, jE, kL, av, l, F, ib, vw, jo, fb, cfb, qt, yy, Ej, aF, LB, es, bE, aS, be, X, us); #c1(MMP2) c2(NP_001289437) c3(72 kDa type IV collagenase isoform 3) c4( ) c5(114212, 114213, 114214, 114215, 114216) c6( ) c7(T, ea, fd, fo, aoj, ci, aAo, aY, Hr, bv, aE, fm, rw, hu, cp, yo, Az, uY, vs, ba, fT, iA, nB, wE, k, lS, vF, aFG, aO, ht, aj, cd, Ak, aEL, EU, fQ, fe, ajz, dB, bOj, Be, ZR, apE, jv, jp, DD, aA, cz, AO, q, bIN, qK, cy, dl, eu, bA, aGP, bf, bg, uU, cl, cfa, pb, cT, rR, iQ, ac, bt, ao, cfe, gV, iW, jh, aHv, fu, ceY, ad, wD, hX, yt, le, lQ, WZ, ff, aeV, iV, bz, lf, lC, iu, bM, xS, if, e, bB, Tz, ip, Na, vV, ja, bc, eG, dH, JI, hl, as, eF, cm, qJ, Kv, ki, vA, vy, wx, in, Vj, rt, dD, n, md, brY, jz, zw, ec, WB, eV, aYF, hi, hQ, yq, SE, YE, abV, PI, hL, at, ut, B, vQ, AS, mr, wL, eX, v, V, hW, nQ, ae, jf, iZ, De, Qr, t, Px, ck, bLr, kP, au, gX, JH, zu, avi, vi, hl, ob, Dd, wV, eq, hd, py, ab, vc, nJ, d, hG, NH, dN, dr, aef, y, hv, aC, fR, azf, lP, al, Vp, u, Bc, K, eL, adT, mQ, wf, hN, g, pL, bwe, uC, aAW, aDs, Gs, uo, Hb, m, QO, cfg, sQ, aw, NT, qy, zP, ft, Lv, By, jl, CN, bMq, ks, cM, Ld, W, JM, Pr, it, fl, L, ceW, fM, fG, j, eA, J, XX, amg, cfd, yg, Nc, bq, kl, xR, Ue, Y, r, en, wA, iK, brZ, hc, dh, dy, Mj, gx, sY, kh, id, hF, iJ, dP, oj, io, hP, adx, WW, uX, lU, lR, xs, eH, cr, Fq, iR, cfF, Bv, sG, ei, hE, tQ, pf, wH, zh, dY, cfc, H, hm, DA, aW, hJ, eM, nw, Xu, vl, Mi, fr, bd, qN, KO, ai, ke, BI, cc, rl, h, dL, aUm, hB, vx, wC, bgf, aq, kp, op, Qy, Qf, hk, bm, hR, uz, te, ceZ, bL, ka, eR, uv, tc, C, uT, aT, af, s, iU, oV, aYp, ajl, nM, pZ, yW, AH, fP, dT, aDj, hS, vr, jE, kL, av, l, F, ib, vw, jo, fb, cfb, qt, yy, Ej, aF, LB, es, bE, aS, be, X, us); #c1(MMP2) c2(NP_004521) c3(72 kDa type IV collagenase isoform 1 preproprotein) c4(147208, 147209, 147210) c5(147211, 147212, 147213, 147214, 147215, 147216) c6( ) c7(T, ea, fd, fo, aoj, ci, aAo, aY, Hr, bv, aE, fm, rw, hu, cp, yo, Az, uY, vs, ba, fT, iA, nB, wE, k, lS, vF, aFG, aO, ht, aj, cd, Ak, aEL, EU, fQ, fe, ajz, dB, bOj, Be, ZR, apE, jv, jp, DD, aA, cz, AO, q, bIN, qK, cy, dl, eu, bA, aGP, bf, bg, uU, cl, cfa, pb, cT, rR, iQ, ac, bt, ao, cfe, gV, iW, jh, aHv, fu, ceY, ad, wD, hX, yt, le, lQ, WZ, ff, aeV, iV, bz, lf, lC, iu, bM, xS, if, e, bB, Tz, ip, Na, vV, ja, bc, eG, dH, Jl, hl, as, eF, cm, qJ, Kv, ki, vA, vy, wx, in, Vj, rt, dD, n, md, brY, jz, zw, ec, WB, eV, aYF, hi, hQ, yq, sE, YE, abV, PI, hL, at, ut, B, vQ, AS, mr, wL, ex, v, V, hW, nQ, ae, jf, iZ, De, Qr, t, Px, ck, bLr, kP, au, gX, JH, zu, avi, vi, hl, ob, Dd, wV, eq, hd, py, ab, vc, nJ, d, hG, NH, dN, dr, aef, y, hv, aC, fR, azf, lP, al, Vp, u, Bc, K, eL, adT, mQ, wf, hN, g, pL, bwe, uC, aAW, aDs, Gs, uo, Hb, m, QO, cfg, sQ, aw, NT, qy, zP, ft, Lv, By, jl, CN, bMq, ks, cM, Ld, W, JM, Pr, it, fl, L, ceW, fM, fG, j, eA, J, XX, amg, cfd, yg, Nc, bq, kl, xR, Ue, Y, r, en, wA, iK, brZ, hc, dh, dy, Mj, gx, sY, kh, id, hF, iJ, dP, oj, io, hP, adx, WW, uX, lU, lR, xs, eH, cr, Fq, iR, cfF, Bv, sG, ei, hE, tQ, pf, wH, zh, dY, cfc, H, hm, DA, aW, hJ, eM, nw, Xu, vl, Mi, fr, bd, qN, KO, ai, ke, BI, cc, rl, h, dL, aUm, hB, vx, wC, bgf, aq, kp, op, Qy, Of, hk, bm, hR, uz, te, ceZ, bL, ka, eR, uv, tc, C, uT, aT, af, s, iU, oV, aYp, ajl, nM, pZ, yW, AH, fP, dT, aDj, hS, vr, jE, kL, av, l, F, ib, vw, jo, fb, cfb, qt, yy, Ej, aF, LB, es, bE, aS, be, X, us); #c1(MMP20) c2(NP_004762) c3(matrix metalloproteinase-20 preproprotein) c4(149393) c5(149394, 149395, 149396, 149397, 149398, 149399, 149400) c6( ) c7(bWJ, ka, ki, ci, y, Ud, cfh); #c1(MMP21) c2(XP_011537559) c3(matrix metalloproteinase-21 isoform X1) c4(335145) c5(335146, 335147, 335148, 335149, 335150, 335151) c6( ) c7(T, aGn, aGo, bB, ci, lQ, B); #c1(MMP24) c2(NP_006681) c3(matrix metalloproteinase-24 preproprotein) c4(167103, 167104) c5(167105, 167106, 167107, 167108, 167109, 167110, 167111, 167112, 167113, 167114, 167115, 167116, 167117, 167118) c6(167119, 167120) c7(d, F, eR, al, aj, eo, ci, aY, V, J); #c1(MMP25) c2(NP_071913) c3(matrix metalloproteinase-25 preproprotein) c4(212282, 212283) c5(212284, 212285, 212286, 212287, 212288, 212289) c6( ) c7(ka, T, al, qy, bB, ci, V, ip, Na, F, aT, Nc, eG, d, aW, Ud, dB, bE, ki, eo, y, X); #c1(MMP25) c2(XP_011520905) c3(matrix metalloproteinase-25 isoform X3) c4( ) c5(315905, 315906, 315907, 315908, 315909, 315910) c6( ) c7(ka, T, al, qy, bB, ci, V, ip, Na, F, aT, Nc, eG, d, aW, Ud, dB, bE, ki, eo, y, X); #c1(MMP26) c2(NP_068573) c3(matrix metalloproteinase-26 preproprotein) c4(209448) c5(209449, 209450, 209451, 209452, 209453, 209454, 209455, 209456, 209457, 209458) c6( ) c7(bL, T, aC, aZ, n, j, ci, ad, aT, lQ, ae, kl, zH, aW, aEl, ff, en, cl, t, bz, hL, lf, it); #c1(MMP28) c2(NP_001027449) c3(matrix metalloproteinase-28 isoform 3 precursor) c4(30123) c5(30124, 30125, 30126, 30127, 30128, 30129, 30130, 30131) c6(30132, 30133) c7(bE, aA, bB, fe, aF, eo, ci, V); #c1(MMP28) c2(NP_077278) c3(matrix metalloproteinase-28 isoform 1 preproprotein) c4(214888) c5(214889, 214890, 214891, 214892, 214893, 214894, 214895, 214896, 214897) c6(214898, 214899) c7(bE, aA, bB, fe, aF, eo, ci, V); #c1(MMP28) c2(NP_116568) c3(matrix metalloproteinase-28 isoform 2 preproprotein) c4(226217, 226218) c5(226219, 226220, 226221, 226222, 226223, 226224, 226225, 226226) c6(226227, 226228) c7(bE, aA, bB, fe, aF, eo, ci, V); #c1(MMP28) c2(XP_011523534) c3(matrix metalloproteinase-28 isoform X5) c4( ) c5(318045, 318046, 318047, 318048, 318049, 318050, 318051) c6(318052, 318053) c7(bE, aA, bB, fe, aF, eo, ci, V); #c1(MMP3) c2(NP_002413) c3(stromelysin-1 preproprotein) c4(128232) c5(128233, 128234, 128235, 128236, 128237, 128238) c6( ) c7(oO, T, fR, tQ, lP, ea, if, e, al, pf, fd, aTy, cL, zh, ci, lR, Tz, Hr, Na, dY, vV, aE, rn, M, K, dQ, eL, bc, eN, cp, hm, eG, aW, hl, hN, g, as, pL, uC, aAW, ba, qJ, fT, eM, lq, vA, mx, vy, uo, m, R, cE, Vj, rt, aNm, dD, pG, Ly, sQ, FG, sw, wJ, aw, ht, n, aj, fr, KO, uE, bd, aCQ, zP, aBP, cd, ai, jz, zw, ec, amJ, rl, kl, sW, lG, dL, ux, he, aqi, wC, A, mR, aJ, fJ, ks, fQ, aq, fe, cfk, dw, cM, sE, op, kT, W, ky, bP, dB, hk, vb, Be, hL, at, uz, ut, bm, YH, anx, bni, it, B, rp, bL, Dw, vQ, eR, fG, oe, jx, aA, lj, ceP, cz, cfl, j, hy, bX, v, V, q, J, eA, aRo, Mg, RI, tL, hb, nX, qp, afQ, ceS, rS, aT, Nc, bK, an, rE, ae, af, dl, eu, bf, s, Y, fO, bg, r, iy, cfm, en, cl, wA, t, iK, bQ, pb, sS, wg, ac, c, yW, lV, hc, gX, uS, ro, cS, np, JH, Bd, rA, VB, vi, sY, kh, am, aFe, aD, iW, jh, vr, oR, mw, Fb, uu, jE, Gn, bKZ, eq, aHv, ad, dZ, hX, dP, bRP, nH, av, F, od, wD, yt, LA, Aa, lQ, jo, WZ, uX, gj, d, lU, cfj, cW, ff, eZ, dN, yy, aF, hp, iV, bz, Hv, ek, LB, uP, gk, es, TA, afb, bQS, lI, X, gZ, aef, wz, aS, bC, be, ei, lf, cfi, gq, y); #c1(MMP7) c2(NP_002414) c3(matrilysin preproprotein) c4(128239) c5(128240, 128241, 128242, 128243, 128244, 128245) c6( ) c7(bM, T, os, aC, pf, ea, if, al, eb, bB, fd, ci, Hr, wa, aE, HR, K, hu, eL, cp, akl, Qe, aa, Jl, aW, az, aB, g, as, eF, uC, aoS, aAW, qJ, fT, eM, Hb, m, fy, iu, dD, gU, wu, sw, ht, NT, n, BH, fr, qN, jz, dL, RT, alw, aq, fe, kp, ar, Qy, W, dB, hL, it, B, bL, AS, dA, fG, lH, uv, aA, cz, cfl, j, cfn, V, q, eA, eJ, qp, qK, aT, Oi, rO, bau, ae, dl, s, NE, nO, oV, Y, ev, r, iy, en, cl, t, kP, yW, nG, gX, cS, dy, KY, ao, gx, sY, id, aD, jh, oZ, Fb, eq, ad, dP, av, F, MS, od, sN, NS, ji, wm, WZ, ff, xs, eH, aF, Fq, iV, bz, LB, bF, D, gk, Bv, bE, adg, lf, X, y); #c1(MMP8) c2(NP_002415) c3(neutrophil collagenase isoform 1 preproprotein) c4(128246) c5(128247, 128248, 128249, 128250, 128251, 128252) c6( ) c7(bM, sY, T, dD, fR, sQ, ea, yk, e, al, bO, fd, aj, j, eq, C, uE, V, cd, J, Tz, wa, eA, av, F, bPx, tL, rl, afQ, aT, dL, lQ, aa, lU, bf, iU, cW, aW, Y, ff, bg, fQ, aFr, afq, aoD, en, pL, g, uC, bz, aZz, op, bm, uP, ba, D, gk, hk, vA, Be, cj, uo, uz, ut, dX, cS, aS, fD, ei, pg, nz, B, y); #c1(MMP8) c2(XP_011541136) c3(neutrophil collagenase isoform X2) c4( ) c5(339670) c6( ) c7(bM, sY, T, dD, fR, sQ, ea, yk, e, al, bO, fd, aj, j, eq, C, uE, V, cd, J, Tz, wa, eA, av, F, bPx, tL, rl, afQ, aT, dL, lQ, aa, lU, bf, iU, cW, aW, Y, ff, bg, fQ, aFr, afq, aoD, en, pL, g, uC, bz, aZz, op, bm, uP, ba, D, gk, hk, vA, Be, cj, uo, uz, ut, dX, cS, aS, fD, ei, pg, nz, B, y); #c1(MMP8) c2(XP_011541137) c3(neutrophil collagenase isoform X1) c4(339671) c5(339672, 339673, 339674, 339675, 339676) c6( ) c7(bM, sY, T, dD, fR, sQ, ea, yk, e, al, bO, fd, aj, j, eq, C, uE, V, cd, J, Tz, wa, eA, av, F, bPx, tL, rl, afQ, aT, dL, lQ, aa, lU, bf, iU, cW, aW, Y, ff, bg, fQ, aFr, afq, aoD, en, pL, g, uC, bz, aZz, op, bm, uP, ba, D, gk, hk, vA, Be, cj, uo, uz, ut, dX, cS, aS, fD, ei, pg, nz, B, y); #c1(MMP8) c2(XP_011541138) c3(neutrophil collagenase isoform X3) c4(339677) c5(339678, 339679, 339680, 339681, 339682, 339683) c6( ) c7(bM, sY, T, dD, fR, sQ, ea, yk, e, al, bO, fd, aj, j, eq, C, uE, V, cd, J, Tz, wa, eA, av, F, bPx, tL, rl, afQ, aT, dL, lQ, aa, lU, bf, iU, cW, aW, Y, ff, bg, fQ, aFr, afq, aoD, en, pL, g, uC, bz, aZz, op, bm, uP, ba, D, gk, hk, vA, Be, cj, uo, uz, ut, dX, cS, aS, fD, ei, pg, nz, B, y); #c1(MMP9) c2(NP_004985) c3(matrix metalloproteinase-9 preproprotein) c4(151696, 151697) c5(151698, 151699, 151700, 151701, 151702, 151703, 151704, 151705, 151706, 151707, 151708, 151709) c6( ) c7(gh, T, ea, lr, fd, fo, aoj, ci, aY, Hr, GK, bv, aE, rw, zy, aFk, hH, cp, aa, ly, yo, az, uY, vs, zE, cfp, fT, nB, cfs, wE, ceR, cj, Zn, k, lS, vF, eg, nD, aFG, vE, aO, ht, ceU, aj, aBP, cd, br, sW, Ak, lG, aNj, ey, pl, fQ, Vt, fe, ajz, dB, bOj, Be, qF, aZy, jp, aA, cz, bX, q, qK, Di, cy, DX, dl, eu, bf, nO, fO, lo, uU, cl, cfa, pb, rR, iM, ac, iQ, ig, bt, rA, ao, On, wi, iW, jh, dG, Gn, fu, ad, afb, hX, yt, Dq, Ot, le, tT, cfr, Aa, lQ, WZ, qZ, cfj, cW, ff, aZw, iV, bz, Ek, aka, lf, lC, aed, iu, oO, bM, lD, xS, aiH, if, e, bB, lK, sD, Tz, ip, Na, bPx, cfq, vV, ja, bc, eG, aiV, aCg, dH, Jl, Du, hl, as, eF, cm, bw, qJ, AV, vA, mx, aiM, tV, vy, cfo, wx, in, Vj, ajy, dD, pG, zt, n, jz, no, zw, zY, ec, gd, eV, cY, oS, A, fJ, lb, hQ, sE, abV, bP, Pl, hL, at, ut, eo, sa, cB, B, arO, vQ, AS, dA, mr, wL, lH, eX, fF, v, V, yw, nQ, ae, jf, kX, afq, acB, t, FT, asw, ck, kP, gX, JH, yH, zu, vi, pY, df, wV, eq, hd, py, od, ds, nJ, mu, d, je, NH, rM, dN, anW, bF, gk, bQS, gZ, aef, MJ, Hg, y, hv, aC, fR, lP, al, bEy, wa, afr, bkA, u, M, B, K, eL, adT, mH, axu, wF, hN, Ku, g, pL, uC, aAW, aik, Gs, uo, m, Ro, P, sQ, sw, aw, NT, fN, qy, pM, zP, biS, ft, aMD, KH, Lv, cU, By, jl, ZN, KV, bkS, cM, kT, W, Pr, it, fl, L, fM, fG, jx, qQ, j, iI, eA, J, XX, Mg, amg, MR, cfd, ceS, yg, qG, Nc, bq, avg, kl, xR, Ue, Y, Pg, r, aHU, en, wA, iK, Dm, Dy, c, ru, hc, Da, cS, dy, fD, Mj, bFR, gx, sY, kh, id, bs, iJ, dP, fU, sN, oj, io, ji, uX, lU, lR, aCt, xs, eH, cr, si, iR, cfF, qa, TA, Bv, sG, bC, ei, hE, tQ, pf, eb, ajp, zh, bWN, dY, H, hm, aW, OZ, eM, uJ, mk, R, vl, ajm, tp, Mi, fr, bd, qN, kO, ai, eW, Bl, rl, h, dL, aUm, ux, hB, wC, aq, kp, mo, op, lx, Qf, NL, hk, vb, Vy, bm, hR, uz, cn, bL, Dw, eR, yk, dk, uv, sM, cR, aBo, OY, C, uT, qp, afQ, aT, bK, af, s, iU, amF, ajl, sS, nM, yW, wq, im, Bd, lJ, aDj, hS, aD, vr, Fb, jE, av, l, F, ib, vw, TC, cfb, afe, qt, eZ, afr, Ej, aF, uF, ek, LB, es, RV, bE, z, HN, aS, XO, be, X, us); #c1(MMRN1) c2(NP_031377) c3(multimerin-1 precursor) c4(172070, 172071) c5(172072, 172073, 172074, 172075, 172076, 172077, 172078, 172079, 172080, 172081, 172082, 172083, 172084, 172085, 172086, 172087, 172088, 172089, 172090, 172091, 172092, 172093) c6(172094, 172095, 172096, 172097, 172098, 172099) c7(T, dD, xu, ea, aw, aj, j, eq, J, dY, M, an, bpU, hm, lR, r, kD, cft, iV, kT, ac, vy, dX, aS, act, gq); #c1(MMRN2) c2(NP_079032) c3(multimerin-2 precursor) c4(216672) c5(216673, 216674, 216675, 216676, 216677, 216678, 216679, 216680, 216681, 216682, 216683, 216684, 216685, 216686, 216687, 216688) c6(216689, 216690, 216691, 216692) c7(T); #c1(MMRN2) c2(XP_005270210) c3(multimerin-2 isoform X1) c4(283288) c5( ) c6( ) c7(T); #c1(MMS19) c2(NP_001276332) c3(MMS19 nucleotide excision repair protein homolog isoform 2) c4(108094, 108095, 108096) c5(108097, 108098, 108099, 108100, 108101, 108102, 108103, 108104, 108105, 108106, 108107, 108108) c6( ) c7(aC, aE, eM, hG, eH, m, g, eF, V); #c1(MMS19) c2(NP_001276334) c3(MMS19 nucleotide excision repair protein homolog isoform 1) c4(108109, 108110) c5(108111, 108112, 108113, 108114, 108115, 108116, 108117, 108118, 108119, 108120, 108121, 108122, 108123, 108124, 108125, 108126, 108127, 108128) c6( ) c7(aC, aE, eM, hG, eH, m, g, eF, V); #c1(MMS19) c2(XP_006718008) c3(MMS19 nucleotide excision repair protein homolog isoform X4) c4( ) c5(291440, 291441, 291442, 291443, 291444, 291445, 291446, 291447) c6( ) c7(aC, aE, eM, hG, eH, m, g, eF, V); #c1(MMS19) c2(XP_011538365) c3(MMS19 nucleotide excision repair protein homolog isoform X5) c4(336056) c5(336057, 336058, 336059, 336060, 336061) c6( ) c7(aC, aE, eM, hG, eH, m, g, eF, V); #c1(MMS22L) c2(NP_940870) c3(protein MMS22-like) c4(260009) c5(260010, 260011, 260012, 260013, 260014, 260015) c6( ) c7(T, K); #c1(MN1) c2(NP_002421) c3(probable tumor suppressor protein MN1) c4(128319) c5(128320, 128321, 128322, 128323, 128324, 128325, 128326) c6( ) c7(boY, iT, gm, bX, cfu, bt); #c1(MNAT1) c2(NP_001171434) c3(CDK-activating kinase assembly factor MAT1 isoform 2) c4(70080) c5(70081, 70082, 70083, 70084, 70085, 70086) c6( ) c7(T, hl, aj, V, mC, aR, J, hX, av, AW, hb, dV, aT, af, pi, uQ, A, aW, Y, eH, Pg, eZ, as, eF, cm, bz, fq, kT, WO, ac, dB, WP, U, Xa, fL, WX, xg, B, ek); #c1(MNAT1) c2(NP_002422) c3(CDK-activating kinase assembly factor MAT1 isoform 1) c4(128327, 128328) c5(128329, 128330, 128331, 128332, 128333, 128334, 128335) c6( ) c7(T, hl, aj, V, mC, aR, J, hX, av, AW, hb, dV, aT, af, pi, uQ, A, aW, Y, eH, Pg, eZ, as, eF, cm, bz, fq, kT, WO, ac, dB, WP, U, Xa, fL, WX, xg, B, ek); #c1(MNAT1) c2(XP_011535097) c3(CDK-activating kinase assembly factor MAT1 isoform X3) c4( ) c5(331829, 331830, 331831, 331832, 331833, 331834) c6( ) c7(T, hl, aj, V, mC, aR, J, hX, av, AW, hb, dV, aT, af, pi, uQ, A, aW, Y, eH, Pg, eZ, as, eF, cm, bz, fq, kT, WO, ac, dB, WP, U, Xa, fL, WX, xg, B, ek); #c1(MNDA) c2(NP_002423) c3(myeloid cell nuclear differentiation antigen) c4(128336, 128337) c5(128338, 128339, 128340, 128341, 128342, 128343, 128344, 128345, 128346, 128347, 128348, 128349, 128350) c6( ) c7(dD, fM, A, bm, cj, eZ, jh, eq, fu, ey, bw, bt, br); #c1(MNS1) c2(NP_060835) c3(meiosis-specific nuclear structural protein 1) c4(198045) c5(198046, 198047, 198048, 198049, 198050, 198051) c6( ) c7(oe); #c1(MNT) c2(NP_064706) c3(max-binding protein MNT) c4(203502) c5(203503, 203504, 203505, 203506, 203507, 203508, 203509, 203510, 203511, 203512, 203513) c6( ) c7(Na, jf, T, aW, Y, Az, ja, aT, K, bdS, B); #c1(MNX1) c2(NP_001158727) c3(motor neuron and pancreas homeobox protein 1 isoform 2) c4(63729) c5(63730, 63731, 63732, 63733, 63734, 63735) c6( ) c7(eX, aj, j, ci, J, mj, mh, bxn, Bj, eV, iY, Gc, ck, c, iT, bYB, bt, agw, cj, mg, cfv, BV, ZW, B, cfw); #c1(MNX1) c2(NP_005506) c3(motor neuron and pancreas homeobox protein 1 isoform 1) c4(156912, 156913) c5(156914, 156915, 156916, 156917, 156918, 156919, 156920, 156921, 156922, 156923, 156924, 156925, 156926) c6( ) c7(ex, aj, j, ci, J, mj, mh, bxn, Bj, eV, iY, Gc, ck, c, iT, bYB, bt, agw, cj, mg, cfv, BV, ZW, B, cfw); #c1(MOAP1) c2(NP_071434) c3(modulator of apoptosis 1) c4(211648) c5(211649, 211650, 211651, 211652, 211653, 211654, 211655, 211656, 211657, 211658) c6( ) c7(T, WP, aj, ae, af, J, ad); #c1(MOB1A) c2(NP_060691) c3(MOB kinase activator 1A) c4(197183, 197184) c5(197185, 197186, 197187, 197188, 197189, 197190) c6( ) c7(av, T, eM, r, bB, v, bz, q); #c1(MOB1B) c2(NP_775739) c3(MOB kinase activator 1B isoform 2) c4(250133) c5(250134, 250135, 250136, 250137, 250138, 250139) c6( ) c7(T); #c1(MOB2) c2(NP_001165694) c3(MOB kinase activator 2 isoform 1) c4(67878) c5(67879, 67880, 67881, 67882, 67883, 67884) c6( ) c7(iR, aq, B); #c1(MOB3B) c2(NP_079037) c3(MOB kinase activator 3B) c4(216709) c5(216710, 216711, 216712, 216713, 216714, 216715, 216716) c6( ) c7(y, T, c); #c1(MOB4) c2(NP_001094289) c3(MOB-like protein phocein isoform 3) c4(40673) c5(40674, 40675, 40676, 40677, 40678, 40679) c6( ) c7(av, T, eM, r, v, bz); #c1(MOB4) c2(NP_001191023) c3(MOB-like protein phocein isoform 2) c4( ) c5(80556, 80557, 80558, 80559, 80560, 80561, 80562, 80563, 80564, 80565, 80566, 80567, 80568, 80569) c6( ) c7(av, T, eM, r, v, bz); #c1(MOB4) c2(NP_056202) c3(MOB-like protein phocein isoform 1) c4(186302) c5(186303, 186304, 186305, 186306, 186307, 186308, 186309, 186310) c6( ) c7(av, T, eM, r, v, bz); #c1(MOBP) c2(NP_001265251) c3(myelin-associated oligodendrocyte basic protein isoform a) c4( ) c5(98064) c6( ) c7(act); #c1(MOBP) c2(NP_001265252) c3(myelin-associated oligodendrocyte basic protein isoform b) c4(98065, 98066) c5(98067, 98068) c6( ) c7(act); #c1(MOBP) c2(NP_891980) c3(myelin-associated oligodendrocyte basic protein isoform c) c4(257346) c5(257347, 257348, 257349, 257350, 257351, 257352, 257353, 257354, 257355, 257356, 257357, 257358, 257359, 257360, 257361, 257362, 257363, 257364, 257365, 257366, 257367, 257368, 257369, 257370, 257371, 257372, 257373, 257374, 257375, 257376, 257377, 257378, 257379, 257380, 257381, 257382, 257383, 257384, 257385, 257386, 257387, 257388, 257389, 257390, 257391) c6( ) c7(act); #c1(MOCOS) c2(NP_060417) c3(molybdenum cofactor sulfurase) c4(195851) c5(195852, 195853, 195854, 195855, 195856, 195857) c6( ) c7(x, cc, fM, nB, nv, aW, bE, hl, Xn, aT, aA, h, fe, aF, m, k, t, Bu, afb, mu); #c1(MOCS1) c2(NP_001068566) c3(molybdenum cofactor biosynthesis protein 1 isoform 4) c4(34574) c5(34575, 34576, 34577, 34578, 34579) c6( ) c7(cfx); #c1(MOCS1) c2(NP_005934) c3(molybdenum cofactor biosynthesis protein 1 isoform 1) c4(160517, 160518) c5(160519, 160520, 160521, 160522, 160523, 160524, 160525, 160526, 160527, 160528, 160529, 160530, 160531, 160532) c6( ) c7(cfx); #c1(MOCS1) c2(XP_011512934) c3(molybdenum cofactor biosynthesis protein 1 isoform X1) c4(304340, 304341) c5(304342, 304343, 304344, 304345, 304346, 304347, 304348, 304349, 304350, 304351, 304352, 304353, 304354, 304355, 304356) c6( ) c7(cfx); #c1(MOCS1) c2(XP_011512935) c3(molybdenum cofactor biosynthesis protein 1 isoform X2) c4(304357, 304358) c5(304359, 304360, 304361, 304362, 304363, 304364, 304365, 304366, 304367, 304368, 304369, 304370) c6( ) c7(cfx); #c1(MOCS1) c2(XP_011512936) c3(molybdenum cofactor biosynthesis protein 1 isoform X3) c4(304371) c5( ) c6( ) c7(cfx); #c1(MOCS1) c2(XP_011512937) c3(molybdenum cofactor biosynthesis protein 1 isoform X4) c4( ) c5(304372, 304373, 304374, 304375, 304376, 304377) c6( ) c7(cfx); #c1(MOCS2) c2(NP_004522) c3(molybdopterin synthase catalytic subunit large subunit MOCS2B) c4(147217, 147218) c5(147219, 147220, 147221, 147222, 147223, 147224, 147225, 147226, 147227, 147228, 147229, 147230, 147231, 147232, 147233) c6( ) c7(cfy, iu); #c1(MOCS2) c2(NP_789776) c3(molybdopterin synthase sulfur carrier subunit small subunit MOCS2A) c4(252250) c5(252251, 252252, 252253, 252254, 252255, 252256, 252257, 252258, 252259, 252260, 252261, 252262, 252263, 252264, 252265) c6( ) c7(cfy, iu); #c1(MOG) c2(NP_001008229) c3(myelin-oligodendrocyte glycoprotein isoform alpha3 precursor) c4(22937, 22938, 22939) c5(22940, 22941, 22942, 22943, 22944, 22945, 22946, 22947, 22948, 22949, 22950, 22951, 22952, 22953, 22954, 22955, 22956, 22957, 22958, 22959, 22960, 22961, 22962, 22963, 22964, 22965, 22966, 22967, 22968, 22969, 22970, 22971, 22972, 22973, 22974, 22975, 22976, 22977, 22978, 22979, 22980, 22981, 22982, 22983, 22984, 22985, 22986, 22987, 22988, 22989, 22990, 22991, 22992, 22993, 22994, 22995, 22996, 22997, 22998, 22999, 23000, 23001, 23002, 23003, 23004, 23005, 23006, 23007, 23008, 23009, 23010, 23011, 23012, 23013) c6( ) c7(bJq, aiR, adK, mF, V, ai, hX, ke, cF, oz, akJ, rE, hH, dF, alY, nP, A, eZ, cfz, bN, U, eo, y, abW, qc); #c1(MOG) c2(NP_001008230) c3(myelin-oligodendrocyte glycoprotein isoform beta2 precursor) c4(23014, 23015, 23016, 23017, 23018) c5(23019, 23020, 23021, 23022, 23023, 23024, 23025, 23026, 23027, 23028, 23029, 23030, 23031, 23032, 23033, 23034, 23035, 23036, 23037, 23038, 23039, 23040, 23041, 23042, 23043, 23044, 23045, 23046, 23047, 23048, 23049, 23050, 23051, 23052, 23053, 23054, 23055, 23056, 23057, 23058, 23059, 23060, 23061, 23062, 23063, 23064, 23065, 23066, 23067, 23068, 23069, 23070, 23071, 23072, 23073, 23074, 23075, 23076, 23077, 23078, 23079, 23080, 23081, 23082, 23083, 23084, 23085, 23086, 23087, 23088, 23089, 23090, 23091, 23092, 23093, 23094, 23095, 23096, 23097, 23098, 23099, 23100, 23101, 23102, 23103, 23104, 23105, 23106, 23107, 23108, 23109, 23110, 23111, 23112, 23113, 23114, 23115, 23116, 23117, 23118, 23119, 23120, 23121, 23122, 23123) c6( ) c7(bJq, aiR, adK, mF, V, ai, hX, ke, cF, oz, akJ, rE, hH, dF, alY, nP, A, eZ, cfz, bN, U, eo, y, abW, qc); #c1(MOG) c2(NP_001163889) c3(myelin-oligodendrocyte glycoprotein isoform alpha6 precursor) c4(66482) c5(66483, 66484, 66485, 66486, 66487, 66488) c6( ) c7(bJq, aiR, adK, mF, V, ai, hX, ke, cF, oz, akJ, rE, hH, dF, alY, nP, A, eZ, cfz, bN, U, eo, y, abW, qc); #c1(MOG) c2(NP_002424) c3(myelin-oligodendrocyte glycoprotein isoform beta1 precursor) c4(128351, 128352, 128353) c5( ) c6( ) c7(bJq, aiR, adK, mF, V, ai, hX, ke, cF, oz, akJ, rE, hH, dF, alY, nP, A, eZ, cfz, bN, U, eo, y, abW, qc); #c1(MOG) c2(NP_996532) c3(myelin-oligodendrocyte glycoprotein isoform alpha1 precursor) c4(264059, 264060, 264061, 264062, 264063) c5(264064, 264065, 264066, 264067, 264068, 264069, 264070, 264071, 264072, 264073, 264074, 264075, 264076, 264077, 264078, 264079, 264080, 264081, 264082, 264083, 264084, 264085, 264086, 264087, 264088, 264089, 264090, 264091, 264092, 264093, 264094, 264095, 264096, 264097, 264098, 264099, 264100, 264101, 264102, 264103, 264104, 264105, 264106, 264107, 264108, 264109, 264110, 264111, 264112, 264113, 264114, 264115, 264116, 264117, 264118, 264119, 264120, 264121, 264122, 264123, 264124, 264125, 264126, 264127, 264128, 264129, 264130, 264131, 264132, 264133, 264134, 264135, 264136, 264137, 264138, 264139, 264140, 264141, 264142, 264143, 264144, 264145, 264146, 264147, 264148, 264149, 264150, 264151, 264152, 264153, 264154, 264155, 264156, 264157, 264158, 264159, 264160, 264161, 264162, 264163, 264164, 264165, 264166, 264167, 264168, 264169, 264170, 264171) c6( ) c7(bJq, aiR, adK, mF, V, ai, hX, ke, cF, oz, akJ, rE, hH, dF, alY, nP, A, eZ, cfz, bN, U, eo, y, abW, qc); #c1(MOG) c2(NP_996533) c3(myelin-oligodendrocyte glycoprotein isoform beta5 precursor) c4(264172, 264173, 264174) c5( ) c6( ) c7(bJq, aiR, adK, mF, V, ai, hX, ke, cF, oz, akJ, rE, hH, dF, alY, nP, A, eZ, cfz, bN, U, eo, y, abW, qc); #c1(MOG) c2(NP_996534) c3(myelin-oligodendrocyte glycoprotein isoform beta3 precursor) c4(264175, 264176, 264177) c5( ) c6( ) c7(bJq, aiR, adK, mF, V, ai, hX, ke, cF, oz, akJ, rE, hH, dF, alY, nP, A, eZ, cfz, bN, U, eo, y, abW, qc); #c1(MOG) c2(NP_996535) c3(myelin-oligodendrocyte glycoprotein isoform alpha2 precursor) c4(264178, 264179, 264180, 264181) c5(264182, 264183, 264184, 264185, 264186, 264187, 264188, 264189, 264190, 264191, 264192, 264193, 264194, 264195, 264196, 264197, 264198, 264199, 264200, 264201, 264202, 264203, 264204, 264205, 264206, 264207, 264208, 264209, 264210, 264211, 264212, 264213, 264214, 264215, 264216, 264217, 264218, 264219, 264220, 264221, 264222, 264223, 264224, 264225, 264226, 264227, 264228, 264229, 264230, 264231, 264232, 264233, 264234, 264235, 264236, 264237, 264238, 264239, 264240, 264241, 264242, 264243) c6( ) c7(bJq, aiR, adK, mF, V, ai, hX, ke, cF, oz, akJ, rE, hH, dF, alY, nP, A, eZ, cfz, bN, U, eo, y, abW, qc); #c1(MOG) c2(NP_996537) c3(myelin-oligodendrocyte glycoprotein isoform alpha5 precursor) c4(264244, 264245, 264246) c5(264247, 264248, 264249, 264250, 264251, 264252, 264253, 264254, 264255, 264256, 264257, 264258, 264259, 264260, 264261, 264262, 264263, 264264, 264265, 264266, 264267, 264268, 264269, 264270, 264271, 264272, 264273, 264274, 264275, 264276, 264277, 264278, 264279, 264280, 264281, 264282, 264283) c6( ) c7(bJq, aiR, adK, mF, V, ai, hX, ke, cF, oz, akJ, rE, hH, dF, alY, nP, A, eZ, cfz, bN, U, eo, y, abW, qc); #c1(MOG) c2(XP_005249188) c3(myelin-oligodendrocyte glycoprotein isoform X1) c4( ) c5(269056, 269057, 269058, 269059, 269060, 269061) c6( ) c7(bJq, aiR, adK, mF, V, ai, hX, ke, cF, oz, akJ, rE, hH, dF, alY, nP, A, eZ, cfz, bN, U, eo, y, abW, qc); #c1(MOG) c2(XP_005249196) c3(myelin-oligodendrocyte glycoprotein isoform X2) c4(269062) c5(269063, 269064, 269065, 269066, 269067, 269068, 269069, 269070, 269071, 269072, 269073, 269074, 269075, 269076, 269077, 269078, 269079, 269080, 269081, 269082, 269083, 269084, 269085, 269086, 269087, 269088, 269089, 269090, 269091, 269092) c6( ) c7(bJq, aiR, adK, mF, V, ai, hX, ke, cF, oz, akJ, rE, hH, dF, alY, nP, A, eZ, cfz, bN, U, eo, y, abW, qc); #c1(MOG) c2(XP_005272877) c3(myelin-oligodendrocyte glycoprotein isoform X1) c4(284207) c5(284208, 284209, 284210, 284211, 284212, 284213, 284214, 284215, 284216, 284217, 284218, 284219, 284220, 284221, 284222, 284223, 284224, 284225, 284226, 284227, 284228, 284229, 284230, 284231, 284232) c6( ) c7(bJq, aiR, adK, mF, V, ai, hX, ke, cF, oz, akJ, rE, hH, dF, alY, nP, A, eZ, cfz, bN, U, eo, y, abW, qc); #c1(MOG) c2(XP_005275309) c3(myelin-oligodendrocyte glycoprotein isoform X1) c4(285609) c5( ) c6( ) c7(bJq, aiR, adK, mF, V, ai, hX, ke, cF, oz, akJ, rE, hH, dF, alY, nP, A, eZ, cfz, bN, U, eo, y, abW, qc); #c1(MOGAT1) c2(NP_477513) c3(2-acylglycerol O-acyltransferase 1) c4(230793) c5(230794, 230795, 230796, 230797, 230798, 230799) c6( ) c7(T, A, aj, v, V, ae, J, ad); #c1(MOGAT2) c2(NP_079374) c3(2-acylglycerol O-acyltransferase 2) c4(217827, 217828, 217829) c5(217830, 217831, 217832, 217833, 217834, 217835, 217836, 217837, 217838, 217839, 217840, 217841, 217842, 217843, 217844, 217845, 217846, 217847) c6( ) c7(dv, fc, dq, v, mf, cP); #c1(MOGAT3) c2(NP_001274076) c3(2-acylglycerol O-acyltransferase 3 isoform b) c4(106015) c5(106016, 106017, 106018, 106019, 106020, 106021) c6( ) c7(aW, v, aq, aT); #c1(MOGAT3) c2(NP_835470) c3(2-acylglycerol O-acyltransferase 3 isoform a) c4(253547) c5(253548, 253549, 253550, 253551, 253552, 253553) c6( ) c7(aW, v, aq, aT); #c1(MOGAT3) c2(XP_005250366) c3(2-acylglycerol O-acyltransferase 3 isoform X1) c4(270268) c5(270269, 270270, 270271, 270272, 270273, 270274) c6( ) c7(aW, v, aq, aT); #c1(MOGS) c2(NP_001139630) c3(mannosyl-oligosaccharide glucosidase isoform 2) c4( ) c5(59578) c6( ) c7(Nq, ck, adj, Nr, x, BV, dV, pg, cfA, wR, Xm, Km, k, ZW, cm, bt, bz, bEa, ao); #c1(MOGS) c2(NP_006293) c3(mannosyl-oligosaccharide glucosidase isoform 1) c4(163739, 163740, 163741) c5(163742, 163743, 163744, 163745, 163746, 163747, 163748, 163749, 163750, 163751, 163752, 163753, 163754, 163755, 163756, 163757, 163758) c6( ) c7(Nq, ck, adj, Nr, x, BV, dV, pg, cfA, wR, Xm, Km, k, ZW, cm, bt, bz, bEa, ao); #c1(MOK) c2(NP_055041) c3(MAPK/MAK/MRK overlapping kinase isoform 1) c4(178500) c5(178501, 178502, 178503, 178504, 178505, 178506) c6( ) c7(bL, T, dD, eR, ea, e, aw, eX, fd, aA, cR, KO, cz, dG, j, aj, v, eq, fr, wy, V, J, ad, fU, av, ns, M, aT, cy, eV, wo, K, wt, H, ae, af, sj, aa, uX, A, lR, s, bf, cW, aW, iU, cr, iy, aF, g, bu, bz, bN, kT, rR, eM, eD, X, vy, at, uz, aS, dy, m, Lz, fD, y, KY, B); #c1(MOK) c2(XP_006720289) c3(MAPK/MAK/MRK overlapping kinase isoform X3) c4( ) c5(293344) c6( ) c7(bL, T, dD, eR, ea, e, aw, eX, fd, aA, cR, KO, cz, dG, j, aj, v, eq, fr, wy, V, J, ad, fU, av, ns, M, aT, cy, eV, wo, K, wt, H, ae, af, sj, aa, uX, A, lR, s, bf, cW, aW, iU, cr, iy, aF, g, bu, bz, bN, kT, rR, eM, eD, X, vy, at, uz, aS, dy, m, Lz, fD, y, KY, B); #c1(MOK) c2(XP_011535355) c3(MAPK/MAK/MRK overlapping kinase isoform X4) c4(332403) c5(332404, 332405, 332406, 332407, 332408) c6( ) c7(bL, T, dD, eR, ea, e, aw, eX, fd, aA, cR, KO, cz, dG, j, aj, v, eq, fr, wy, V, J, ad, fU, av, ns, M, aT, cy, eV, wo, K, wt, H, ae, af, sj, aa, uX, A, lR, s, bf, cW, aW, iU, cr, iy, aF, g, bu, bz, bN, kT, rR, eM, eD, X, vy, at, uz, aS, dy, m, Lz, fD, y, KY, B); #c1(MON1A) c2(NP_001135973) c3(vacuolar fusion protein MON1 homolog A isoform b) c4(53872) c5(53873, 53874, 53875, 53876, 53877, 53878) c6( ) c7(aUW); #c1(MON1A) c2(NP_115731) c3(vacuolar fusion protein MON1 homolog A isoform a) c4(223731) c5(223732, 223733, 223734, 223735, 223736, 223737) c6( ) c7(aUW); #c1(MON1B) c2(NP_001273568) c3(vacuolar fusion protein MON1 homolog B isoform 2) c4(105309) c5(105310, 105311, 105312, 105313, 105314, 105315) c6( ) c7(eW, ac); #c1(MON1B) c2(NP_001273569) c3(vacuolar fusion protein MON1 homolog B isoform 3) c4(105316) c5(105317, 105318, 105319, 105320, 105321, 105322) c6( ) c7(eW, ac); #c1(MON1B) c2(NP_055755) c3(vacuolar fusion protein MON1 homolog B isoform 1) c4(183308, 183309) c5(183310, 183311, 183312, 183313, 183314, 183315, 183316, 183317, 183318, 183319, 183320, 183321) c6( ) c7(eW, ac); #c1(MON2) c2(NP_001265398) c3(protein MON2 homolog isoform 2) c4(98512, 98513, 98514) c5(98515, 98516, 98517, 98518, 98519, 98520, 98521, 98522, 98523, 98524, 98525, 98526) c6( ) c7(adz, aDs); #c1(MON2) c2(NP_001265399) c3(protein MON2 homolog isoform 3) c4( ) c5(98527, 98528, 98529, 98530, 98531, 98532) c6( ) c7(adz, aDs); #c1(MON2) c2(NP_055841) c3(protein MON2 homolog isoform 1) c4(183833, 183834) c5(183835, 183836, 183837, 183838, 183839, 183840, 183841, 183842, 183843, 183844, 183845, 183846, 183847) c6( ) c7(adz, aDs); #c1(MORC3) c2(NP_056173) c3(MORC family CW-type zinc finger protein 3) c4(186123) c5(186124, 186125, 186126, 186127, 186128, 186129) c6( ) c7(cm, Bv); #c1(MORF4L1) c2(NP_001252534) c3(mortality factor 4-like protein 1 isoform 3) c4( ) c5(94203, 94204, 94205, 94206, 94207, 94208, 94209, 94210, 94211) c6( ) c7(aW, T, qK, K, aT); #c1(MORF4L1) c2(NP_006782) c3(mortality factor 4-like protein 1 isoform 1) c4(168021, 168022) c5(168023, 168024, 168025, 168026, 168027, 168028, 168029, 168030, 168031, 168032, 168033, 168034, 168035, 168036, 168037) c6( ) c7(aW, T, qK, K, aT); #c1(MORF4L1) c2(NP_996670) c3(mortality factor 4-like protein 1 isoform 2) c4(264359, 264360) c5(264361, 264362, 264363, 264364, 264365, 264366) c6( ) c7(aW, T, qK, K, aT); #c1(MORN1) c2(NP_001287989) c3(MORN repeat-containing protein 1 isoform 2) c4(113304) c5(113305, 113306, 113307, 113308, 113309, 113310) c6( ) c7(kl); #c1(MORN1) c2(XP_011540474) c3(MORN repeat-containing protein 1 isoform X5) c4( ) c5(338940, 338941, 338942, 338943, 338944, 338945) c6( ) c7(kl); #c1(MORN1) c2(XP_011540479) c3(MORN repeat-containing protein 1 isoform X10) c4( ) c5(338946, 338947, 338948, 338949, 338950, 338951, 338952, 338953, 338954, 338955, 338956, 338957) c6( ) c7(kl); #c1(MORN2) c2(NP_001138922) c3(MORN repeat-containing protein 2) c4(58207) c5(58208, 58209, 58210, 58211, 58212, 58213, 58214, 58215, 58216, 58217, 58218, 58219, 58220, 58221) c6( ) c7(c); #c1(MORN5) c2(NP_940871) c3(MORN repeat-containing protein 5 isoform a) c4(260016) c5(260017, 260018, 260019, 260020, 260021, 260022) c6( ) c7(Na, Nc); #c1(MORN5) c2(XP_005251934) c3(MORN repeat-containing protein 5 isoform X2) c4(271307, 271308) c5(271309, 271310, 271311, 271312, 271313, 271314) c6( ) c7(Na, Nc); #c1(MORN5) c2(XP_005251935) c3(MORN repeat-containing protein 5 isoform X4) c4( ) c5(271315) c6( ) c7(Na, Nc); #c1(MORN5) c2(XP_011516760) c3(MORN repeat-containing protein 5 isoform X5) c4( ) c5(310219, 310220, 310221, 310222, 310223) c6( ) c7(Na, Nc); #c1(MOS) c2(NP_005363) c3(proto-oncogene serine/threonine-protein kinase mos) c4(155447) c5(155448, 155449, 155450, 155451, 155452, 155453) c6( ) c7(T, fM, wu, jh, bs, aA, fr, ci, qN, hF, br, afb, x, nv, aT, h, wm, iU, aW, hl, aF, cm, bw, bz, ck, eM, nB, iA, iT, eD, m, k, auk, bt); #c1(MOV1OL1) c2(NP_001157576) c3 (putative helicase Mov1Ol1 isoform 2) c4(62267) c5(62268, 62269, 62270, 62271, 62272, 62273) c6( ) c7(Kg); #c1(MOV1OL1) c2(NP_001157577) c3 (putative helicase Mov1Ol1 isoform 3) c4(62274, 62275, 62276) c5(62277, 62278, 62279, 62280, 62281, 62282, 62283, 62284, 62285, 62286, 62287, 62288) c6( ) c7(Kg); #c1(MOV1OL1) c2(NP_001157578) c3(putative helicase Mov1Ol1 isoform 4) c4( ) c5(62289, 62290, 62291, 62292, 62293, 62294, 62295, 62296, 62297, 62298, 62299, 62300, 62301, 62302, 62303) c6( ) c7(Kg); #c1(MOV1OL1) c2(NP_061868) c3(putative helicase Mov1Ol1 isoform 1) c4(200854, 200855) c5(200856, 200857, 200858, 200859, 200860, 200861, 200862, 200863, 200864, 200865, 200866) c6( ) c7(Kg); #c1(MPC1) c2(NP_001257808) c3(mitochondrial pyruvate carrier 1 isoform 2) c4( ) c5(95646, 95647, 95648, 95649, 95650) c6( ) c7(cfB, lB); #c1(MPC1) c2(NP_057182) c3(mitochondrial pyruvate carrier 1 isoform 1) c4(189333, 189334, 189335) c5(189336, 189337, 189338, 189339, 189340, 189341, 189342, 189343, 189344, 189345, 189346, 189347, 189348) c6( ) c7(cfB, lB); #c1(MPC2) c2(NP_001137146) c3(mitochondrial pyruvate carrier 2) c4(54973, 54974) c5(54975, 54976, 54977, 54978, 54979, 54980, 54981, 54982, 54983, 54984, 54985, 54986, 54987, 54988, 54989, 54990) c6( ) c7(bA, cz); #c1(MPDU1) c2(NP_004861) c3(mannose-P-dolichol utilization defect 1 protein) c4(150385, 150386, 150387) c5(150388, 150389, 150390, 150391, 150392, 150393, 150394, 150395, 150396, 150397, 150398) c6( ) c7(SQ, cfC, dv, at, dL, ey, Km); #c1(MPDU1) c2(XP_006721660) c3(mannose-P-dolichol utilization defect 1 protein isoform X1) c4(294195, 294196) c5(294197, 294198, 294199, 294200, 294201, 294202, 294203, 294204, 294205, 294206, 294207, 294208, 294209) c6( ) c7(SQ, cfC, dv, at, dL, ey, Km); #c1(MPDU1) c2(XP_006721662) c3(mannose-P-dolichol utilization defect 1 protein isoform X3) c4( ) c5(294210, 294211, 294212, 294213, 294214, 294215) c6( ) c7(SQ, cfC, dv, at, dl, ey, Km); #c1(MPDZ) c2(NP_001248335) c3(multiple PDZ domain protein isoform 2) c4(93700) c5(93701, 93702, 93703, 93704, 93705, 93706, 93707) c6( ) c7(wQ, aap, lR, bZY, cfD, FX, Hr); #c1(MPDZ) c2(NP_001248336) c3(multiple PDZ domain protein isoform 3) c4(93708) c5(93709, 93710, 93711, 93712, 93713, 93714) c6( ) c7(wQ, aap, lR, bZY, cfD, FX, Hr); #c1(MPDZ) c2(NP_003820) c3(multiple PDZ domain protein isoform 1) c4(141148) c5(141149, 141150, 141151, 141152, 141153, 141154, 141155, 141156, 141157, 141158) c6( ) c7(wQ, aap, lR, bZY, cfD, FX, Hr); #c1(MPDZ) c2(XP_005251680) c3(multiple PDZ domain protein isoform X6) c4(271029, 271030) c5(271031, 271032, 271033, 271034, 271035, 271036, 271037, 271038, 271039, 271040, 271041, 271042) c6( ) c7(wQ, aap, lR, bZY, cfD, FX, Hr); #c1(MPDZ) c2(XP_006716950) c3(multiple PDZ domain protein isoform X4) c4(290598) c5(290599, 290600, 290601, 290602, 290603, 290604, 290605, 290606, 290607, 290608) c6( ) c7(wQ, aap, lR, bZY, cfD, FX, Hr); #c1(MPEG1) c2(NP_001034485) c3(macrophage-expressed gene 1 protein precursor) c4(31753, 31754) c5(31755, 31756, 31757, 31758, 31759, 31760, 31761, 31762, 31763, 31764, 31765, 31766) c6( ) c7(oO, T, lE, d, af, jp, kg, aaH, hb, Y, aT, aA, dq, bNj, Lq, ati, kp, af, iK, wS, aLI); #c1(MPG) c2(NP_001015052) c3(DNA-3-methyladenine glycosylase isoform b) c4(26164) c5(26165, 26166, 26167, 26168, 26169, 26170) c6( ) c7(bM, T, os, dD, am, ea, jh, aA, bB, fd, oZ, rf, eq, ci, V, q, ad, er, eA, Kx, iu, hd, aE, aT, h, dL, K, ox, ae, N, jf, d, bf, s, iU, aW, aml, Y, Lf, ff, r, aq, Pp, aF, en, bu, iV, t, bz, iP, ck, es, Ta, ZL, byx, eM, iA, bE, iT, aRk, cj, U, dX, m, Kj, ER, pw, be, bt, B, y, Tc); #c1(MPG) c2(NP_001015054) c3(DNA-3-methyladenine glycosylase isoform c) c4( ) c5(26171, 26172, 26173, 26174, 26175, 26176, 26177, 26178, 26179, 26180, 26181, 26182, 26183) c6( ) c7(bM, T, os, dD, am, ea, jh, aA, bB, fd, oZ, rf, eq, ci, V, q, ad, er, eA, Kx, iu, hd, aE, aT, h, dL, K, ox, ae, N, jf, d, bf, s, iU, aW, aml, Y, Lf, ff, r, aq, Pp, aF, en, bu, iV, t, bz, iP, ck, es, Ta, ZL, byx, eM, iA, bE, iT, aRk, cj, U, dX, m, Kj, ER, pw, be, bt, B, y, Tc); #c1(MPG) c2(NP_002425) c3(DNA-3-methyladenine glycosylase isoform a) c4(128354, 128355, 128356) c5(128357, 128358, 128359, 128360, 128361, 128362, 128363, 128364) c6( ) c7(bM, T, os, dD, am, ea, jh, aA, bB, fd, oZ, rf, eq, ci, V, q, ad, er, eA, Kx, iu, hd, aE, aT, h, dL, K, ox, ae, N, jf, d, bf, s, iU, aW, aml, Y, Lf, ff, r, aq, Pp, aF, en, bu, iV, t, bz, iP, ck, es, Ta, ZL, byx, eM, iA, bE, iT, aRk, cj, U, dX, m, Kj, ER, pw, be, bt, B, y, Tc); #c1(MPHOSPH10) c2(NP_005782) c3(U3 small nucleolar ribonucleoprotein protein MPP10) c4(159300) c5(159301, 159302, 159303, 159304, 159305, 159306) c6( ) c7(aW, aT); #c1(MPHOSPH6) c2(NP_005783) c3(M-phase phosphoprotein 6) c4(159307) c5(159308, 159309, 159310, 159311, 159312, 159313) c6( ) c7(T, sS, Hx, FO, FP, cz, K, an, cm, fq, af); #c1(MPHOSPH8) c2(NP_059990) c3(M-phase phosphoprotein 8) c4(193525) c5(193526, 193527, 193528, 193529, 193530, 193531, 193532, 193533, 193534, 193535, 193536, 193537) c6( ) c7(ci); #c1(MPHOSPH8) c2(XP_005266493) c3(M-phase phosphoprotein 8 isoform X2) c4(280192) c5( ) c6( ) c7(ci); #c1(MPHOSPH8) c2(XP_011533426) c3(M-phase phosphoprotein 8 isoform X1) c4(329538, 329539) c5(329540, 329541, 329542, 329543, 329544, 329545, 329546, 329547, 329548, 329549, 329550, 329551) c6( ) c7(ci); #c1(MPHOSPH9) c2(NP_073619) c3(M-phase phosphoprotein 9) c4(213166) c5(213167, 213168, 213169, 213170, 213171, 213172) c6( ) c7(cm, V); #c1(MPHOSPH9) c2(XP_011536041) c3(M-phase phosphoprotein 9 isoform X2) c4(333345) c5(333346, 333347, 333348, 333349, 333350, 333351) c6( ) c7(cm, V); #c1(MPI) c2(NP_001276084) c3(mannose-6-phosphate isomerase isoform 2) c4(107904) c5(107905, 107906, 107907, 107908, 107909, 107910) c6( ) c7(bL, cfF, cfE, aiV, bo, aoE, dv, hl, brC, dX, xH, Km, abQ, t, cfG, ccM); #c1(MPI) c2(NP_001276085) c3(mannose-6-phosphate isomerase isoform 3) c4( ) c5(107911, 107912, 107913, 107914, 107915) c6( ) c7(bL, cfF, cfE, aiV, bo, aoE, dv, hl, brC, dX, xH, Km, abQ, t, cfG, ccM); #c1(MPI) c2(NP_001276086) c3(mannose-6-phosphate isomerase isoform 4) c4(107916) c5(107917, 107918, 107919, 107920, 107921, 107922) c6( ) c7(bL, cfF, cfE, aiV, bo, aoE, dv, hl, brC, dX, xH, Km, abQ, t, cfG, ccM); #c1(MPI) c2(NP_002426) c3(mannose-6-phosphate isomerase isoform 1) c4(128365) c5(128366, 128367, 128368, 128369, 128370, 128371) c6( ) c7(bL, cfF, cfE, aiV, bo, aoE, dv, hl, brC, dX, xH, Km, abQ, t, cfG, ccM); #c1(MPI) c2(XP_011519895) c3(mannose-6-phosphate isomerase isoform X2) c4(314680) c5(314681) c6( ) c7(bL, cfF, cfE, aiV, bo, aoE, dv, hl, brC, dX, xH, Km, abQ, t, cfG, ccM); #c1(MPLK1P) c2(NP_619646) c3(M-phase-specific PLK1-interacting protein) c4(237000) c5(237001, 237002, 237003, 237004, 237005, 237006) c6( ) c7(aBq, nl, nh, bnA); #c1(MPO) c2(NP_000241) c3(myeloperoxidase precursor) c4(5395, 5396) c5(5397, 5398, 5399, 5400, 5401, 5402, 5403, 5404, 5405, 5406, 5407, 5408) c6( ) c7(oO, bM, T, aC, fR, ea, e, al, bO, fd, bB, aoj, qj, ci, aY, fw, sD, ye, Tz, x, gJ, dY, aE, pp, K, hu, hH, eL, Zm, aa, aW, az, hl, mQ, nY, kt, aB, g, Fu, aBa, ve, bw, cfl, ba, ou, fT, eM, iT, U, cj, k, m, iu, dD, aKm, lo, ht, NT, aw, n, aj, oY, BF, uE, qN, cd, br, b, ft, hw, ec, lG, Gw, hn, dL, wb, OV, anP, fe, aq, op, ar, iO, dB, Qu, at, E, fL, qF, lB, Pr, B, L, bL, bmT, fM, eR, yk, jx, rf, aA, cR, cz, hy, v, j, GC, C, azF, uW, V, q, aZZ, eJ, cF, qp, dn, aT, bOk, yu, cy, jj, bq, lM, ae, af, eu, s, iU, oV, fO, bg, r, lo, en, t, ck, wg, ac, hA, hc, hO, dy, cS, EY, fD, cfH, bt, rA, aDj, ao, Kd, Gl, am, qb, aD, iW, df, oA, jh, Gn, eq, hF, mC, ad, KL, dP, av, F, sN, Ot, ds, iY, ji, vP, iL, uX, NP, bJV, ff, eZ, yy, hv, and, aF, Sm, iR, iV, bz, aYt, ek, TA, D, gk, atM, bE, uR, eB, lI, aS, Gu, oQ, i, y, kS, ne, rk); #c1(MPP1) c2(NP_001159933) c3(55 kDa erythrocyte membrane protein isoform 3) c4(64993) c5(64994, 64995, 64996, 64997, 64998, 64999) c6( ) c7(av, A, F, xE, dV, h, g, zy, y, bt, amW, uQ); #c1(MPP1) c2(NP_001159934) c3(55 kDa erythrocyte membrane protein isoform 4) c4(65000) c5(65001, 65002, 65003, 65004, 65005, 65006) c6( ) c7(av, A, F, xE, dV, h, g, zy, y, bt, amW, uQ); #c1(MPP1) c2(NP_002427) c3(55 kDa erythrocyte membrane protein isoform 1) c4(128372) c5(128373, 128374, 128375, 128376, 128377, 128378) c6( ) c7(av, A, F, xE, dV, h, g, zy, y, bt, amW, uQ); #c1(MPP2) c2(NP_001265299) c3(MAGUK p55 subfamily member 2 isoform 1) c4(98222, 98223, 98224) c5(98225, 98226, 98227, 98228, 98229) c6( ) c7(WA, es, qw, lg, aT); #c1(MPP2) c2(NP_001265301) c3(MAGUK p55 subfamily member 2 isoform 4) c4(98230, 98231) c5(98232, 98233, 98234, 98235, 98236, 98237) c6( ) c7(WA, es, qw, lg, aT); #c1(MPP2) c2(NP_001265305) c3(MAGUK p55 subfamily member 2 isoform 8) c4(98238) c5(98239) c6( ) c7(WA, es, qw, lg, aT); #c1(MPP2) c2(NP_005365) c3(MAGUK p55 subfamily member 2 isoform 3) c4(155454, 155455) c5(155456, 155457, 155458, 155459, 155460, 155461, 155462, 155463, 155464, 155465) c6( ) c7(WA, es, qw, lg, aT); #c1(MPP2) c2(XP_011523127) c3(MAGUK p55 subfamily member 2 isoform X3) c4( ) c5(317637, 317638, 317639, 317640, 317641, 317642, 317643, 317644, 317645, 317646, 317647, 317648, 317649, 317650, 317651, 317652, 317653, 317654, 317655, 317656, 317657, 317658, 317659, 317660, 317661, 317662) c6( ) c7(WA, es, qw, lg, aT); #c1(MPP3) c2(NP_001923) c3(MAGUK p55 subfamily member 3) c4(122233) c5(122234, 122235, 122236, 122237, 122238) c6( ) c7(d, qy, nf, nA, aR, bN); #c1(MPP3) c2(XP_011523130) c3(MAGUK p55 subfamily member 3 isoform X2) c4(317663, 317664) c5(317665, 317666, 317667, 317668, 317669, 317670, 317671, 317672, 317673, 317674, 317675, 317676, 317677) c6( ) c7(d, qy, nf, nA, aR, bN); #c1(MPP5) c2(NP_001243479) c3(MAGUK p55 subfamily member 5 isoform 2) c4( ) c5(90919, 90920, 90921, 90922, 90923, 90924, 90925, 90926, 90927, 90928) c6( ) c7(nw, aIF); #c1(MPP5) c2(NP_071919) c3(MAGUK p55 subfamily member 5 isoform 1) c4(212298, 212299, 212300) c5(212301, 212302, 212303, 212304, 212305, 212306, 212307, 212308) c6( ) c7(nw, aIF); #c1(MPP7) c2(XP_005252425) c3(MAGUK p55 subfamily member 7 isoform X2) c4(271781) c5(271782, 271783, 271784, 271785, 271786, 271787) c6( ) c7(bM, e, EQ, lB, fq, af); #c1(MPP7) c2(XP_011517640) c3 (MAGUK p55 subfamily member 7 isoform X3) c4(311477) c5(311478, 311479, 311480, 311481, 311482, 311483) c6( ) c7(bM, e, EQ, lB, fq, af); #c1(MPPE1) c2(NP_001229833) c3(metallophosphoesterase 1 isoform 2) c4(85389) c5(85390, 85391, 85392, 85393, 85394, 85395) c6( ) c7(uJ, bK); #c1(MPPE1) c2(NP_075563) c3(metallophosphoesterase 1 isoform 1) c4(213959, 213960) c5(213961, 213962, 213963, 213964, 213965, 213966, 213967, 213968, 213969, 213970, 213971) c6( ) c7(uJ, bK); #c1(MPPE1) c2(XP_006722408) c3(metallophosphoesterase 1 isoform X3) c4(294697) c5(294698, 294699, 294700, 294701, 294702, 294703) c6( ) c7(uJ, bK); #c1(MPPE1) c2(XP_011524034) c3(metallophosphoesterase 1 isoform X5) c4(318554) c5(318555, 318556, 318557, 318558, 318559, 318560, 318561, 318562, 318563, 318564, 318565) c6( ) c7(uJ, bK); #c1(MPPED2) c2(NP_001138871) c3(metallophosphoesterase MPPED2 isoform 2) c4(57989, 57990) c5(57991, 57992, 57993, 57994, 57995, 57996) c6( ) c7(T, eR, ki, aw, fe, aqK, cm, cd); #c1(MPPED2) c2(XP_005253169) c3(metallophosphoesterase MPPED2 isoform X1) c4(272233) c5(272234, 272235, 272236, 272237, 272238, 272239) c6( ) c7(T, eR, ki, aw, fe, aqK, cm, cd); #c1(MPPED2) c2(XP_005253171) c3(metallophosphoesterase MPPED2 isoform X3) c4( ) c5(272240, 272241, 272242, 272243, 272244, 272245) c6( ) c7(T, eR, ki, aw, fe, aqK, cm, cd); #c1(MPRIP) c2(NP_055949) c3(myosin phosphatase Rho-interacting protein isoform 1) c4(184616) c5(184617, 184618, 184619, 184620, 184621, 184622, 184623) c6( ) c7(dD, d, aAF, ea, ceC, cj, fe, nw, eq, wb, Hp); #c1(MPRIP) c2(NP_958431) c3(myosin phosphatase Rho-interacting protein isoform 2) c4(262060) c5(262061, 262062, 262063, 262064, 262065, 262066, 262067, 262068, 262069) c6( ) c7(dD, d, aAF, ea, ceC, cj, fe, nw, eq, wb, Hp); #c1(MPRIP) c2(XP_005256620) c3(myosin phosphatase Rho-interacting protein isoform X6) c4(274232) c5(274233, 274234, 274235, 274236, 274237, 274238, 274239, 274240, 274241, 274242, 274243, 274244, 274245, 274246) c6( ) c7(dD, d, aAF, ea, ceC, cj, fe, nw, eq, wb, Hp); #c1(MPRIP) c2(XP_005256621) c3(myosin phosphatase Rho-interacting protein isoform X9) c4(274247, 274248) c5(274249, 274250, 274251, 274252, 274253, 274254, 274255, 274256, 274257, 274258, 274259, 274260) c6( ) c7(dD, d, aAF, ea, ceC, cj, fe, nw, eq, wb, Hp); #c1(MPRIP) c2(XP_011522063) c3(myosin phosphatase Rho-interacting protein isoform X1) c4(316718) c5(316719, 316720, 316721, 316722, 316723, 316724) c6( ) c7(dD, d, aAF, ea, ceC, cj, fe, nw, eq, wb, Hp); #c1(MPRIP) c2(XP_011522064) c3(myosin phosphatase Rho-interacting protein isoform X2) c4(316725) c5(316726, 316727, 316728, 316729, 316730) c6( ) c7(dD, d, aAF, ea, ceC, cj, fe, nw, eq, wb, Hp); #c1(MPRIP) c2(XP_011522065) c3(myosin phosphatase Rho-interacting protein isoform X3) c4(316731) c5( ) c6( ) c7(dD, d, aAF, ea, ceC, cj, fe, nw, eq, wb, Hp); #c1(MPRIP) c2(XP_011522066) c3(myosin phosphatase Rho-interacting protein isoform X4) c4(316732) c5(316733, 316734, 316735, 316736, 316737, 316738) c6( ) c7(dD, d, aAF, ea, ceC, cj, fe, nw, eq, wb, Hp); #c1(MPST) c2(NP_001123989) c3(3-mercaptopyruvate sulfurtransferase isoform 2) c4( ) c5(48148, 48149, 48150, 48151, 48152, 48153, 48154, 48155, 48156, 48157, 48158, 48159, 48160, 48161, 48162, 48163, 48164, 48165, 48166, 48167, 48168, 48169, 48170) c6( ) c7(bL, x, eM, cfJ, aW, aT, k, C, pd, B, hq); #c1(MPST) c2(NP_066949) c3(3-mercaptopyruvate sulfurtransferase isoform 1) c4(207644, 207645, 207646) c5(207647, 207648, 207649, 207650, 207651, 207652, 207653, 207654, 207655, 207656, 207657) c6( ) c7(bL, x, eM, cfJ, aW, aT, k, C, pd, B, hq); #c1(MPV17) c2(XP_005264383) c3(protein Mpv17 isoform X1) c4(278882, 278883, 278884, 278885) c5(278886, 278887, 278888, 278889, 278890, 278891, 278892, 278893, 278894, 278895, 278896, 278897, 278898, 278899, 278900, 278901, 278902, 278903, 278904, 278905, 278906, 278907, 278908, 278909, 278910, 278911, 278912, 278913) c6( ) c7(atx, dD, cfK, ob, eR, ea, qx, cz, aNG, ah, eq, aY, aoz, hb, btl, vw, K, wt, cb, afJ, apn, beV, hJ, cm, kC, bw, mG, dy, cfL, aSQ, aw, VK); #c1(MPV17) c2(XP_005264384) c3(protein Mpv17 isoform X3) c4( ) c5(278914, 278915, 278916, 278917, 278918, 278919, 278920, 278921, 278922, 278923, 278924, 278925) c6( ) c7(atx, dD, cfK, ob, eR, ea, qx, cz, aNG, ah, eq, aY, aoz, hb, btl, vw, K, wt, cb, afJ, apn, beV, hJ, cm, kC, bw, mG, dy, cfL, aSQ, awl, VK); #c1(MPV17) c2(XP_006712084) c3(protein Mpv17 isoform X2) c4(286385, 286386, 286387, 286388) c5(286389, 286390, 286391, 286392, 286393, 286394, 286395, 286396, 286397, 286398, 286399, 286400) c6( ) c7(atx, dD, cfK, ob, eR, ea, qx, cz, aNG, ah, eq, aY, aoz, hb, btl, vw, K, wt, cb, afJ, apn, beV, hJ, cm, kC, bw, mG, dy, cfL, aSQ, awl, VK); #c1(MPV17L2) c2(NP_116072) c3(mpv17-like protein 2 precursor) c4(225325) c5(225326, 225327, 225328, 225329, 225330, 225331) c6( ) c7(V); #c1(MPZ) c2(NP_000521) c3(myelin protein PO precursor) c4( ) c5(11670, 11671, 11672, 11673, 11674, 11675, 11676, 11677, 11678, 11679, 11680, 11681, 11682, 11683, 11684, 11685, 11686, 11687, 11688) c6( ) c7(T, ob, aiK, Pt, cfN, xF, bkY, amp, bbn, My, yk, agU, n, fu, bX, mF, V, q, aR, Hx, od, no, uT, vJ, cfQ, HC, UD, cfP, ji, K, bUo, bU, bV, an, UF, AT, aCM, bT, WQ, aFT, r, aDm, cfM, iV, cbm, cm, bMj, pb, cfO, dr, sk, sS, Ew, ql, bkW, bgy, c, yW, AH, bk, R, cfR, bCG, afO); #c1(MPZL1) c2(NP_001139663) c3(myelin protein zero-like protein 1 isoform c precursor) c4(59653) c5(59654, 59655, 59656, 59657, 59658, 59659) c6( ) c7(zQ, c, yS, cz, ci, zP, B, bA); #c1(MPZL1) c2(NP_003944) c3(myelin protein zero-like protein 1 isoform a precursor) c4(142370) c5(142371, 142372, 142373, 142374, 142375, 142376) c6( ) c7(zQ, c, yS, cz, ci, zP, B, bA); #c1(MPZL1) c2(NP_078845) c3(myelin protein zero-like protein 1 isoform b precursor) c4(215675) c5(215676, 215677, 215678, 215679, 215680, 215681) c6( ) c7(zQ, c, yS, cz, ci, zP, B, bA); #c1(MPZL2) c2(NP_658911) c3(myelin protein zero-like protein 2 precursor) c4(239602) c5(239603, 239604, 239605, 239606, 239607, 239608, 239609, 239610, 239611, 239612) c6( ) c7(wg, aW, aMT, aT, j, ale, dr); #c1(MPZL3) c2(NP_001273081) c3(myelin protein zero-like protein 3 isoform b precursor) c4(104306) c5(104307, 104308, 104309, 104310, 104311, 104312) c6( ) c7(dr); #c1(MPZL3) c2(NP_938016) c3(myelin protein zero-like protein 3 isoform a precursor) c4(259519) c5(259520, 259521, 259522, 259523, 259524, 259525) c6( ) c7(dr); #c1(MRI) c2(NP_001181928) c3(major histocompatibility complex class 1-related gene protein isoform 2 precursor) c4(74429) c5(74430, 74431, 74432, 74433, 74434, 74435, 74436) c6( ) c7(ahR, hd, aE, nB, K, iV, aR); #c1(MRI) c2(NP_001181929) c3(major histocompatibility complex class 1-related gene protein isoform 3 precursor) c4(74437) c5(74438, 74439, 74440, 74441, 74442, 74443, 74444) c6( ) c7(ahR, hd, aE, nB, K, iV, aR); #c1(MRI) c2(NP_00181964) c3(major histocompatibility complex class 1-related gene protein isoform 4 precursor) c4(74445) c5(74446, 74447, 74448, 74449, 74450, 74451, 74452) c6( ) c7(ahR, hd, aE, nB, K, iV, aR); #c1(MRI) c2(NP_001522) c3(major histocompatibility complex class 1-related gene protein isoform 1 precursor) c4(118593) c5(118594, 118595, 118596, 118597, 118598, 118599, 118600) c6( ) c7(ahR, hd, aE, nB, K, iV, aR); #c1(MRAP) c2(NP_848932) c3(melanocortin-2 receptor accessory protein isoform alpha) c4(254237) c5(254238, 254239, 254240, 254241, 254242, 254243, 254244, 254245, 254246, 254247) c6( ) c7(aK, mR, ld, fe, ahr, Ph, ai, cfS); #c1(MRAP) c2(NP_996781) c3(melanocortin-2 receptor accessory protein isoform beta) c4(264516) c5(264517, 264518, 264519, 264520, 264521, 264522, 264523, 264524, 264525, 264526) c6( ) c7(aK, mR, ld, fe, ahr, Ph, ai, cfS); #c1(MRAP2) c2(XP_011533703) c3(melanocortin-2 receptor accessory protein 2 isoform X1) c4(329796) c5(329797, 329798, 329799, 329800, 329801, 329802) c6( ) c7(iv, v, j, sj); #c1(MRAS) c2(NP_001239019) c3(ras-related protein M-Ras isoform 1 precursor) c4(88168, 88169, 88170) c5(88171, 88172, 88173, 88174, 88175, 88176, 88177, 88178, 88179, 88180, 88181, 88182, 88183, 88184, 88185) c6( ) c7(dY, aw, r, j, an, q); #c1(MRAS) c2(NP_001239022) c3(ras-related protein M-Ras isoform 2 precursor) c4( ) c5(88186, 88187, 88188, 88189, 88190) c6( ) c7(dY, aw, r, j, an, q); #c1(MRAS) c2(XP_005247286) c3(ras-related protein M-Ras isoform X2) c4( ) c5(267745, 267746, 267747, 267748, 267749, 267750) c6( ) c7(dY, aw, r, j, an, q); #c1(MRC1) c2(NP_002429) c3(macrophage mannose receptor 1 precursor) c4(128379, 128380, 128381) c5(128382, 128383, 128384, 128385, 128386, 128387, 128388, 128389, 128390, 128391, 128392, 128393, 128394, 128395, 128396, 128397, 128398, (28399) c6( ) c7(ID, bM, T, os, aC, fd, ajp, bB, ci, aub, AW, afr, aE, pX, K, DY, eL, ly, aW, dg, g, as, eE, eM, m, gU, NT, n, aGU, fr, qN, ai, hw, gd, KN, KH, auS, dL, pI, ajV, OV, oS, fJ, ask, fe, CO, zZ, acD, Ld, lx, RB, acC, hk, hL, at, oh, it, B, EM, fG, yk, aA, Do, q, eA, eJ, XX, mj, aT, ae, qw, Uf, s, NE, Y, Lf, RF, r, en, bu, cl, t, iK, bN, ck, sS, iM, ig, id, jh, KO, oZ, pI, ad, hX, dP, avp, YQ, Yi, uQ, d, Gy, BO, je, ff, cr, af, adj, bz, dr, bF, Ew, bE, y, X); #c1(MRC2) c2(NP_006030) c3(C-type mannose receptor 2 precursor) c4(161282, 161283, 161284) c5(161285, 161286, 161287, 161288, 161289, 161290, 161291, 161292, 161293, 161294, 161295, 161296) c6( ) c7(zQ, qt, ff, aT, ci, en, zP, eA); #c1(MRE11A) c2(NP_005581) c3(double-strand break repair protein MRE11A isoform 2) c4(157629) c5(157630, 157631, 157632, 157633, 157634, 157635, 157636, 157637, 157638, 157639, 157640, 157641, 157642, 157643, 157644, 157645, 157646, 157647, 157648, 157649, 157650, 157651, 157652, 157653) c6( ) c7(T, os, aC, fR, WL, iW, jh, aA, bB, fd, oZ, Gn, bRy, ci, bX, V, q, ad, eA, iu, cc, Bj, rA, aT, dL, K, kU, eL, af, B, BO, aix, s, aW, qt, r, aF, g, cl, t, QK, bz, dr, adq, ck, ky, dB, bE, m, bC, y, bt, X); #c1(MRE11A) c2(NP_005582) c3(double-strand break repair protein MRE11A isoform 1) c4(157654, 157655, 157656, 157657) c5(157658, 157659, 157660, 157661, 157662, 157663, 157664, 157665, 157666, 157667, 157668, 157669) c6( ) c7(T, os, aC, fR, WL, iW, jh, aA, bB, fd, oZ, Gn, bRy, ci, bX, V, q, ad, eA, iu, cc, Bj, rA, aT, dL, K, kU, eL, af, B, BO, aix, s, aW, qt, r, aF, g, cl, t, QK, bz, dr, adq, ck, ky, dB, bE, m, bC, y, bt, X); #c1(MRE11A) c2(XP_006718905) c3(double-strand break repair protein MRE11A isoform X2) c4(292051, 292052) c5(292053, 292054, 292055, 292056, 292057, 292058, 292059) c6( ) c7(T, os, aC, fR, WL, iW, jh, aA, bB, fd, oZ, Gn, bRy, ci, bX, V, q, ad, eA, iu, cc, Bj, rA, aT, dL, K, kU, eL, af, B, BO, aix, s, aW, qt, r, aF, g, cl, t, QK, bz, dr, adq, ck, ky, dB, bE, m, bC, y, bt, X); #c1(MREG) c2(NP_060470) c3(melanoregulin) c4(196087, 196088) c5(196089, 196090, 196091, 196092, 196093, 196094, 196095, 196096, 196097, 196098, 196099, 196100, 196101, 196102, 196103, 196104) c6( ) c7(at, sj); #c1(MRFAP1) c2(NP_150638) c3(MORF4 family-associated protein 1 isoform a) c4(228210) c5(228211, 228212, 228213, 228214, 228215, 228216, 228217, 228218, 228219, 228220, 228221, 228222, 228223, 228224) c6( ) c7(zb, g, hS); #c1(MRGBP) c2(NP_060740) c3(MRG/MORF4L-binding protein) c4(197562) c5(197563, 197564, 197565, 197566, 197567, 197568) c6( ) c7(T, s, r, ci, adg, q); #c1(MRGPRF) c2(NP_659452) c3(mas-related G-protein coupled receptor member F) c4(239993, 239994) c5(239995, 239996, 239997, 239998, 239999, 240000, 240001, 240002, 240003, 240004, 240005, 240006, 240007, 240008, 240009, 240010) c6( ) c7(ck, fU, TI, ajT, eR, NL, zs, h, aq, lo, yq, nA, iR, bt); #c1(MRGPRX1) c2(NP_671732) c3(mas-related G-protein coupled receptor member XI) c4(242181) c5(242182, 242183, 242184, 242185, 242186, 242187) c6( ) c7(bL, bM, d, aE, Y, pX, ff, h, Ja, v, dX, en, he, Fg, cE); #c1(MRGPRX3) c2(NP_473372) c3(mas-related G-protein coupled receptor member X3) c4(230473, 230474) c5(230475, 230476, 230477, 230478, 230479, 230480, 230481, 230482, 230483, 230484, 230485, 230486) c6( ) c7(bL, bM, d, aE, Y, pX, ff, h, Ja, v, dX, en, he, Fg, cE); #c1(MRGPRX4) c2(NP_473373) c3(mas-related G-protein coupled receptor member X4) c4(230487) c5(230488, 230489, 230490, 230491, 230492, 230493) c6( ) c7(bL, bM, d, aE, Y, pX, ff, h, Ja, v, dX, en, he, Fg, cE); #c1(MRI1) c2(NP_001026897) c3(methylthioribose-1-phosphate isomerase isoform 1) c4(29933) c5(29934, 29935, 29936, 29937, 29938, 29939, 29940, 29941) c6(29942, 29943) c7(By); #c1(MRI1) c2(NP_115661) c3(methylthioribose-I-phosphate isomerase isoform 2) c4(223484) c5(223485, 223486, 223487, 223488, 223489, 223490) c6( ) c7(By); #c1(MRM1) c2(NP_079140) c3(rRNA methyltransferase 1, mitochondrial precursor) c4(217208) c5(217209, 217210, 217211, 217212, 217213, 217214) c6( ) c7(Na); #c1(MRO) c2(NP_001120647) c3(protein maestro isoform c) c4(44351) c5(44352, 44353, 44354, 44355, 44356, 44357) c6( ) c7(qJ, T, no, AS, Db, cj, Fx, ci, hf, AN); #c1(MRO) c2(NP_001120648) c3 (protein maestro isoform d) c4(44358) c5(44359, 44360, 44361, 44362, 44363, 44364) c6( ) c7(qJ, T, no, AS, Db, cj, Fx, ci, hf, AN); #c1(MRO) c2(XP_005258424) c3 (protein maestro isoform X1) c4(275658) c5(275659, 275660, 275661, 275662, 275663, 275664, 275665, 275666, 275667, 275668) c6( ) c7(qJ, T, no, AS, Db, cj, Fx, ci, hf, AN); #c1(MRO) c2(XP_011524528) c3(protein maestro isoform X2) c4(319099) c5(319100, 319101, 319102, 319103, 319104, 319105, 319106, 319107, 319108, 319109) c6( ) c7(qJ, T, no, AS, Db, cj, Fx, ci, hf, AN); #c1(MROH2B) c2(NP_775760) c3(maestro heat-like repeat-containing protein family member 28) c4(250189, 250190) c5(250191, 250192, 250193, 250194, 250195, 250196, 250197, 250198, 250199, 250200, 250201, 250202) c6( ) c7(V); #c1(MRPL1) c2(NP_064621) c3(39S ribosomal protein L1, mitochondrial precursor) c4(203332, 203333) c5(203334, 203335, 203336, 203337, 203338, 203339, 203340) c6( ) c7(fq, Ea, cz); #c1(MRPL1) c2(XP_011530502) c3(39S ribosomal protein L1, mitochondrial isoform X2) c4( ) c5(326400, 326401, 326402, 326403, 326404) c6( ) c7(fq, Ea, cz); #c1(MRPL10) c2(NP_660298) c3(39S ribosomal protein L10, mitochondrial isoform a precursor) c4(240792) c5(240793, 240794, 240795, 240796, 240797, 240798) c6( ) c7(dD, eq); #c1(MRPL10) c2(NP_683685) c3(39S ribosomal protein L10, mitochondrial isoform b) c4(242369) c5(242370, 242371, 242372, 242373, 242374, 242375, 242376, 242377, 242378, 242379) c6( ) c7(dD, eq); #c1(MRPL11) c2(NP_057134) c3(39S ribosomal protein L11, mitochondrial isoform a) c4(189074) c5(189075, 189076, 189077, 189078, 189079, 189080) c6( ) c7(af); #c1(MRPL11) c2(NP_733934) c3(39S ribosomal protein L11, mitochondrial isoform b) c4(247271) c5(247272, 247273, 247274, 247275, 247276, 247277) c6( ) c7(af); #c1(MRPL11) c2(NP_733935) c3(39S ribosomal protein L11, mitochondrial isoform c) c4(247278) c5(247279, 247280, 247281, 247282, 247283, 247284) c6( ) c7(af); #c1(MRPL13) c2(NP_054797) c3(39S ribosomal protein L13, mitochondrial) c4(177992) c5(177993, 177994, 177995, 177996, 177997, 177998) c6( ) c7(aA, ff, j, en, aT); #c1(MRPL15) c2(NP_054894) c3(39S ribosomal protein L15, mitochondrial) c4(178234) c5(178235, 178236, 178237, 178238, 178239, 178240) c6( ) c7(aj); #c1(MRPL17) c2(NP_071344) c3(39S ribosomal protein L17, mitochondrial) c4(211107) c5(211108, 211109, 211110, 211111, 211112, 211113) c6( ) c7(y); #c1(MRPL19) c2(NP_055578) c3(39S ribosomal protein L19, mitochondrial) c4(182045, 182046) c5(182047, 182048, 182049, 182050, 182051, 182052, 182053, 182054, 182055, 182056, 182057, 182058, 182059, 182060, 182061, 182062) c6( ) c7(yA, aT); #c1(MRPL23) c2(NP_066957) c3(39S ribosomal protein L23, mitochondrial) c4(207742) c5(207743, 207744, 207745, 207746, 207747, 207748, 207749, 207750, 207751, 207752) c6( ) c7(t, aF, m, aA); #c1(MRPL28) c2(XP_011520653) c3(39S ribosomal protein L28, mitochondrial isoform X1) c4(315554, 315555, 315556, 315557, 315558) c5(315559, 315560, 315561, 315562, 315563, 315564, 315565, 315566, 315567, 315568, 315569, 315570, 315571, 315572, 315573, 315574, 315575, 315576, 315577, 315578, 315579, 315580, 315581, 315582, 315583, 315584, 315585, 315586, 315587, 315588, 315589, 315590, 315591, 315592, 315593, 315594, 315595, 315596) c6( ) c7(T, aC, fM, id, ht, jx, jh, aA, fd, j, ci, hF, Fc, q, ad, eA, ip, x, av, iu, fX, ib, nv, kH, aT, h, hn, dL, j, XZ, hu, hS, pq, ae, eL, Zm, d, je, s, aix, iU, aW, Y, ff, r, aF, en, cl, as, t, iK, bw, iP, ck, qJ, iM, bP, eM, aJm, dB, iT, cj, pg, k, bt, B, vi); #c1(MRPL3) c2(NP_009139) c3(39S ribosomal protein L3, mitochondrial) c4(171034, 171035, 171036) c5(171037, 171038, 171039, 171040, 171041, 171042, 171043, 171044, 171045, 171046, 171047, 171048, 171049, 171050, 171051, 171052, 171053, 171054) c6( ) c7(bE, cfT, vc, jh, p); #c1(MRPL33) c2(NP_004882) c3(39S ribosomal protein L33, mitochondrial isoform a) c4(150530, 150531) c5(150532, 150533, 150534, 150535, 150536, 150537) c6( ) c7(v); #c1(MRPL33) c2(NP_663303) c3(39S ribosomal protein L33, mitochondrial isoform b) c4(241157) c5(241158, 241159, 241160, 241161, 241162, 241163) c6( ) c7(v); #c1(MRPL36) c2(NP_115868) c3(39S ribosomal protein L36, mitochondrial) c4( ) c5(224397, 224398, 224399, 224400, 224401, 224402, 224403, 224404, 224405, 224406, 224407, 224408, 224409, 224410) c6( ) c7(os, aW, if, aT, oZ, K, m, ci, qN, t, aed, ae, awc, ad); #c1(MRPL36) c2(XP_011512381) c3(39S ribosomal protein L36, mitochondrial isoform X1) c4(303087) c5( ) c6( ) c7(os, aW, if, aT, oZ, K, m, ci, qN, t, aed, ae, awc, ad); #c1(MRPL36) c2(XP_011512382) c3(39S ribosomal protein L36, mitochondrial isoform X2) c4( ) c5(303088, 303089, 303090, 303091) c6( ) c7(os, aW, if, aT, oZ, K, m, ci, qN, t, aed, ae, awc, ad); #c1(MRPL40) c2(NP_003767) c3(39S ribosomal protein L40, mitochondrial) c4(140678) c5(140679, 140680, 140681, 140682, 140683, 140684) c6( ) c7(agd); #c1(MRPL41) c2(NP_115866) c3(39S ribosomal protein L41, mitochondrial) c4(224390) c5(224391, 224392, 224393, 224394, 224395, 224396) c6( ) c7(T, aW, aT, ci, K, iK, ae, ad); #c1(MRPL44) c2(NP_075066) c3(39S ribosomal protein [44, mitochondrial) c4(213648) c5(213649, 213650, 213651, 213652, 213653, 213654) c6( ) c7(acJ, cfU, ca); #c1(MRPL49) c2(NP_004918) c3(39S ribosomal protein L49, mitochondrial) c4(150775) c5(150776, 150777, 150778, 150779, 150780, 150781, 150782, 150783, 150784, 150785) c6( ) c7(Y); #c1(MRPL52) c2(NP_848026) c3(39S ribosomal protein L52, mitochondrial isoform a) c4(253706) c5(253707, 253708, 253709, 253710, 253711, 253712, 253713, 253714, 253715) c6( ) c7(fq); #c1(MRPL52) c2(NP_851313) c3(39S ribosomal protein L52, mitochondrial isoform c) c4(254534) c5(254535, 254536, 254537, 254538, 254539, 254540) c6( ) c7(fq); #c1(MRPL52) c2(NP_851824) c3(39S ribosomal protein L52, mitochondrial isoform e) c4(254608) c5(254609, 254610, 254611, 254612, 254613, 254614) c6( ) c7(fq); #c1(MRPL52) c2(XP_005267385) c3(39S ribosomal protein L52, mitochondrial isoform X1) c4(280929) c5(280930, 280931, 280932, 280933, 280934, 280935, 280936, 280937, 280938, 280939, 280940, 280941, 280942, 280943, 280944, 280945, 280946) c6( ) c7(fq); #c1(MRPL9) c2(NP_001287662) c3(39S ribosomal protein L9, mitochondrial isoform 2) c4(112477) c5(112478, 112479, 112480, 112481, 112482, 112483, 112484) c6( ) c7(v, aT); #c1(MRPL9) c2(NP_113608) c3(39S ribosomal protein L9, mitochondrial isoform 1) c4(221717) c5(221718, 221719, 221720, 221721, 221722, 221723) c6( ) c7(v, aT); #c1(MRPS11) c2(NP_073750) c3(28S ribosomal protein S11, mitochondrial isoform a) c4(213459) c5(213460, 213461, 213462, 213463, 213464, 213465) c6( ) c7(B, aA); #c1(MRPS11) c2(NP_789775) c3(28S ribosomal protein S11, mitochondrial isoform b) c4(252243) c5(252244, 252245, 252246, 252247, 252248, 252249) c6( ) c7(B, aA); #c1(MRPS12) c2(NP_203527) c3(28S ribosomal protein S12, mitochondrial precursor) c4(228409) c5(228410, 228411, 228412, 228413, 228414, 228415, 228416, 228417, 228418, 228419, 228420, 228421, 228422, 228423) c6( ) c7(aF, aA); #c1(MRPS16) c2(NP_057149) c3(28S ribosomal protein S16, mitochondrial) c4(189121, 189122, 189123, 189124, 189125, 189126, 189127, 189128, 189129, 189130, 189131, 189132, 189133, 189134, 189135, 189136, 189137, 189138, 189139, 189140) c5(189141, 189142, 189143, 189144, 189145, 189146, 189147, 189148, 189149, 189150, 189151, 189152, 189153, 189154, 189155, 189156, 189157, 189158) c6( ) c7(cfV); #c1(MRPS18B) c2(NP_054765) c3(28S ribosomal protein S18b, mitochondrial) c4(177653, 177654) c5(177655, 177656, 177657, 177658, 177659, 177660, 177661, 177662, 177663, 177664, 177665, 177666, 177667, 177668, 177669, 177670, 177671, 177672, 177673, 177674, 177675, 177676, 177677, 177678, 177679, 177680, 177681, 177682, 177683, 177684, 177685, 177686, 177687, 177688, 177689, 177690, 177691, 177692, 177693, 177694, 177695, 177696, 177697, 177698, 177699, 177700, 177701, 177702, 177703) c6( ) c7(eZ, aA); #c1(MRPS18B) c2(XP_005249087) c3(28S ribosomal protein S18b, mitochondrial isoform X1) c4(268956) c5(268957, 268958, 268959, 268960, 268961, 268962, 268963, 268964, 268965, 268966, 268967, 268968, 268969, 268970, 268971, 268972, 268973, 268974, 268975, 268976, 268977, 268978, 268979) c6( ) c7(eZ, aA); #c1(MRPS22) c2(NP_064576) c3(28S ribosomal protein S22, mitochondrial) c4(203048) c5(203049, 203050, 203051, 203052, 203053, 203054, 203055, 203056, 203057, 203058) c6( ) c7(cfW, kC, vc, aT); #c1(MRPS22) c2(XP_005247697) c3(28S ribosomal protein S22, mitochondrial isoform X1) c4(267932, 267933) c5(267934, 267935, 267936, 267937, 267938, 267939, 267940, 267941, 267942, 267943, 267944, 267945) c6( ) c7(cfW, kC, vc, aT); #c1(MRPS22) c2(XP_011511297) c3(28S ribosomal protein S22, mitochondrial isoform X3) c4(301816) c5(301817, 301818, 301819, 301820, 301821, 301822) c6( ) c7(cfW, kC, vc, aT); #c1(MRPS23) c2(NP_057154) c3(28S ribosomal protein S23, mitochondrial) c4(189180) c5(189181, 189182, 189183, 189184, 189185, 189186) c6( ) c7(aT); #c1(MRPS28) c2(NP_054737) c3(28S ribosomal protein S28, mitochondrial) c4(177493) c5(177494, 177495, 177496, 177497, 177498, 177499) c6( ) c7(aT); #c1(MRPS30) c2(NP_057724) c3(28S ribosomal protein S30, mitochondrial) c4(192519) c5(192520, 192521, 192522, 192523, 192524, 192525) c6( ) c7(ck, ant, d, dA, aW, iW, aT, r, la, aj, v, zG, uF, dT, wq, zl, cd, ao); #c1(MRPS31) c2(NP_005821) c3(28S ribosomal protein S31, mitochondrial) c4(159699) c5(159700, 159701, 159702, 159703, 159704, 159705) c6( ) c7(aj, J); #c1(MRPS33) c2(NP_444263) c3(28S ribosomal protein S33, mitochondrial) c4(230175, 230176) c5(230177, 230178, 230179, 230180, 230181, 230182, 230183, 230184, 230185, 230186, 230187, 230188, 230189, 230190, 230191, 230192, 230193, 230194, 230195, 230196, 230197, 230198, 230199, 230200) c6( ) c7(bL); #c1(MRPS6) c2(NP_115865) c3(28S ribosomal protein S6, mitochondrial) c4(224383) c5(224384, 224385, 224386, 224387, 224388, 224389) c6( ) c7(aw, j); #c1(MRPS7) c2(NP_057055) c3(28S ribosomal protein S7, mitochondrial) c4(188655) c5(188656, 188657, 188658, 188659, 188660, 188661) c6( ) c7(aT); #c1(MRPS9) c2(NP_872578) c3(28S ribosomal protein S9, mitochondrial) c4(256404) c5(256405, 256406, 256407, 256408, 256409, 256410) c6( ) c7(bL, aC); #c1(MRRF) c2(NP_001166983) c3(ribosome-recycling factor, mitochondrial isoform 3 precursor) c4(68750) c5(68751, 68752, 68753, 68754, 68755, 68756) c6( ) c7(acJ); #c1(MRRF) c2(XP_011517485) c3(ribosome-recycling factor, mitochondrial isoform X3) c4( ) c5(311233, 311234) c6( ) c7(acJ); #c1(MRRF) c2(XP_011517486) c3(ribosome-recycling factor, mitochondrial isoform X1) c4(311235, 311236, 311237, 311238, 311239, 311240, 311241, 311242) c5(311243, 311244, 311245, 311246, 311247, 311248, 311249, 311250, 311251, 311252, 311253, 311254, 311255, 311256, 311257, 311258) c6( ) c7(acJ); #c1(MRRF) c2(XP_011517487) c3(ribosome-recycling factor, mitochondrial isoform X4) c4( ) c5(311259, 311260, 311261, 311262, 311263, 311264, 311265) c6( ) c7(acJ); #c1(MRRF) c2(XP_011517488) c3(ribosome-recycling factor, mitochondrial isoform X2) c4(311266) c5(311267, 311268, 311269, 311270, 311271, 311272, 311273, 311274, 311275, 311276, 311277, 311278, 311279, 311280, 311281, 311282, 311283, 311284, 311285, 311286, 311287, 311288, 311289, 311290, 311291, 311292, 311293, 311294, 311295, 311296, 311297, 311298, 311299, 311300, 311301, 311302, 311303) c6( ) c7(acJ); #c1(MRS2) c2(NP_001273193) c3(magnesium transporter MRS2 homolog, mitochondrial isoform a) c4(104531, 104532, 104533, 104534) c5(104535, 104536, 104537, 104538, 104539, 104540) c6( ) c7(zw, YE, eR, pY, Do, TG, vj, aaG, aA, cz, wo, v, azg, azh, aF, pL, aY, HI, aAO, aAL); #c1(MRS2) c2(NP_001273194) c3(magnesium transporter MRS2 homolog, mitochondrial isoform c) c4(104541) c5(104542, 104543, 104544, 104545, 104546, 104547) c6( ) c7(zw, YE, eR, pY, Do, TG, vj, aaG, aA, cz, wo, v, azg, azh, aF, pl, aY, HI, aAO, aAL); #c1(MRS2) c2(NP_001273195) c3(magnesium transporter MRS2 homolog, mitochondrial isoform d) c4(104548) c5(104549, 104550, 104551, 104552, 104553, 104554, 104555, 104556, 104557, 104558, 104559, 104560, 104561, 104562, 104563, 104564, 104565, 104566) c6( ) c7(zw, YE, eR, pY, Do, TG, vj, aaG, aA, cz, wo, v, azg, azh, aF, pL, aY, HI, aAO, aAL); #c1(MRS2) c2(NP_065713) c3(magnesium transporter MRS2 homolog, mitochondrial isoform b precursor) c4(205041) c5(205042, 205043, 205044, 205045, 205046, 205047, 205048) c6( ) c7(zw, YE, eR, pY, Do, TG, vj, aaG, aA, cz, wo, v, azg, azh, aF, pL, aY, HI, aAO, aAL); #c1(MRS2) c2(XP_005249299) c3(magnesium transporter MRS2 homolog, mitochondrial isoform X1) c4( ) c5(269153, 269154, 269155, 269156, 269157) c6( ) c7(zw, YE, eR, pY, Do, TG, vj, aaG, aA, cz, wo, v, azg, azh, aF, pl, aY, HI, aAO, aAL); #c1(MRVI1) c2(NP_001092049) c3(protein MRVI1 isoform a) c4(39268, 39269, 39270) c5(39271, 39272, 39273, 39274, 39275, 39276, 39277, 39278, 39279, 39280, 39281, 39282, 39283, 39284, 39285, 39286, 39287, 39288, 39289, 39290, 39291, 39292, 39293, 39294, 39295) c6( ) c7(he, ad); #c1(MRVI1) c2(NP_001093633) c3(protein MRVI1 isoform b) c4(40346, 40347) c5(40348, 40349, 40350, 40351, 40352, 40353, 40354, 40355, 40356, 40357, 40358, 40359, 40360, 40361, 40362, 40363) c6( ) c7(he, ad); #c1(MRVI1) c2(NP_001193809) c3(protein MRVI1 isoform e) c4(82633) c5(82634, 82635, 82636, 82637, 82638, 82639) c6( ) c7(he, ad); #c1(MRVI1) c2(NP_569056) c3(protein MRVI1 isoform d) c4(233603, 233604, 233605) c5(233606, 233607, 233608, 233609, 233610, 233611, 233612, 233613, 233614, 233615, 233616, 233617, 233618, 233619, 233620, 233621, 233622) c6( ) c7(he, ad); #c1(MS4A1) c2(NP_068769) c3(B-lymphocyte antigen CD20) c4(210286, 210287) c5(210288, 210289, 210290, 210291, 210292, 210293, 210294, 210295, 210296, 210297, 210298, 210299, 210300, 210301, 210302, 210303, 210304, 210305, 210306, 210307) c6( ) c7(bM, T, fd, x, DY, pk, aW, axu, mQ, mK, iT, aep, cj, k, gn, fy, aov, aw, yG, zj, aj, aHj, ai, ft, bXL, h, eV, ey, ox, bxg, OV, mR, A, Og, bXK, yq, CA, xe, Wt, kT, ajd, fL, fM, dA, jx, eX, V, q, J, zn, aT, aol, sb, iU, xR, Y, lC, r, pb, OC, lY, ck, cT, wg, kP, iM, Zg, hc, bt, Ze, Ke, ob, iW, cfx, bXN, jh, bS, mC, dP, CJ, fX, qh, amY, aHp, gj, je, Of, eZ, LB, bXM, y, ne); #c1(MS4A12) c2(NP_001157942) c3(membrane-spanning 4-domains subfamily A member 12 isoform 2) c4(62922) c5(62923, 62924, 62925, 62926, 62927, 62928) c6( ) c7(bB, bE); #c1(MS4A12) c2(NP_060186) c3(membrane-spanning 4-domains subfamily A member 12 isoform 1) c4(194652, 194653) c5(194654, 194655, 194656, 194657, 194658, 194659, 194660, 194661, 194662, 194663, 194664, 194665, 194666, 194667) c6( ) c7(bB, bE); #c1(MS4A2) c2(NP_001243845) c3(high affinity immunoglobulin epsilon receptor subunit beta isoform 3) c4( ) c5(91816, 91817, 91818, 91819, 91820) c6( ) c7(bL, bM, Dw, dD, eX, j, eq, pM, Jx, q, fS, eV, Ds, JK, bGD, eu, sp, r, Xm, eE, ve, cT, ac, z, uz, Tu, k, fD); #c1(MS4A2) c2(XP_005273903) c3(high affinity immunoglobulin epsilon receptor subunit beta isoform X2) c4(284874, 284875) c5(284876, 284877) c6( ) c7(bL, bM, Dw, dD, eX, j, eq, pM, Jx, q, fS, eV, Ds, JK, bGD, eu, sp, r, Xm, eE, ve, cT, ac, z, uz, Tu, k, fD); #c1(MS4A2) c2(XP_011543152) c3(high affinity immunoglobulin epsilon receptor subunit beta isoform X1) c4( ) c5(341978, 341979, 341980, 341981, 341982) c6( ) c7(bL, bM, Dw, dD, eX, j, eq, pM, Jx, q, fS, eV, Ds, JK, bGD, eu, sp, r, Xm, eE, ve, cT, ac, z, uz, Tu, k, fD); #c1(MS4A3) c2(NP_001026836) c3(membrane-spanning 4-domains subfamily A member 3 isoform c) c4( ) c5(29640, 29641, 29642, 29643, 29644) c6( ) c7(bM, T, cF, Qu, g, fq); #c1(MS4A3) c2(NP_001026979) c3(membrane-spanning 4-domains subfamily A member 3 isoform b) c4(30033) c5(30034, 30035, 30036, 30037, 30038, 30039, 30040, 30041, 30042, 30043) c6( ) c7(bM, T, cF, Qu, g, fq); #c1(MS4A3) c2(NP_006129) c3(membrane-spanning 4-domains subfamily A member 3 isoform a) c4(162281, 162282) c5(162283, 162284, 162285, 162286, 162287, 162288) c6( ) c7(bM, T, cF, Qu, g, fq); #c1(MS4A3) c2(XP_011543665) c3(membrane-spanning 4-domains subfamily A member 3 isoform X1) c4( ) c5(342771) c6( ) c7(bM, T, cF, Qu, g, fq); #c1(MS4A4A) c2(NP_001230195) c3(membrane-spanning 4-domains subfamily A member 4A isoform 3) c4(86083) c5(86084, 86085, 86086, 86087, 86088, 86089, 86090, 86091, 86092, 86093, 86094) c6( ) c7(ey, fK, af); #c1(MS4A4A) c2(NP_076926) c3(membrane-spanning 4-domains subfamily A member 4A isoform 2) c4( ) c5(214279, 214280, 214281, 214282, 214283, 214284, 214285, 214286) c6( ) c7(ey, fK, af); #c1(MS4A4A) c2(NP_683876) c3(membrane-spanning 4-domains subfamily A member 4A isoform 1) c4(242598, 242599, 242600) c5(242601, 242602, 242603, 242604, 242605, 242606, 242607, 242608) c6( ) c7(ey, fK, af); #c1(MS4A6A) c2(NP_001234928) c3(membrane-spanning 4-domains subfamily A member GA isoform 4) c4(87802, 87803) c5(87804, 87805, 87806, 87807, 87808, 87809, 87810) c6( ) c7(sO, ey, fK, af); #c1(MS4A6A) c2(NP_071744) c3(membrane-spanning 4-domains subfamily A member GA isoform 2) c4( ) c5(211909, 211910, 211911, 211912, 211913) c6( ) c7(sO, ey, fK, af); #c1(MS4A6A) c2(NP_690590) c3(membrane-spanning 4-domains subfamily A member 6A isoform 3) c4(244856) c5(244857, 244858, 244859, 244860, 244861, 244862) c6( ) c7(sO, ey, fK, af); #c1(MS4A6A) c2(NP_690591) c3(membrane-spanning 4-domains subfamily A member GA isoform 1) c4( ) c5(244863, 244864, 244865, 244866, 244867, 244868, 244869, 244870) c6( ) c7(sO, ey, fK, af); #c1(MS4A6A) c2(XP_005274235) c3(membrane-spanning 4-domains subfamily A member 6A isoform X3) c4(284996, 284997) c5(284998, 284999, 285000, 285001, 285002, 285003, 285004, 285005) c6( ) c7(sO, ey, fK, af); #c1(MS4A6A) c2(XP_006718723) c3(membrane-spanning 4-domains subfamily A member GA isoform X4) c4(291994) c5( ) c6( ) c7(sO, ey, fK, af); #c1(MS4A6A) c2(XP_006718724) c3(membrane-spanning 4-domains subfamily A member 6A isoform X5) c4(291995) c5(291996) c6( ) c7(sO, ey, fK, af); #c1(MS4A6A) c2(XP_006718726) c3(membrane-spanning 4-domains subfamily A member GA isoform X7) c4( ) c5(291997, 291998, 291999, 292000, 292001) c6( ) c7(sO, ey, fK, af); #c1(MS4A6A) c2(XP_011543511) c3(membrane-spanning 4-domains subfamily A member 6A isoform X8) c4( ) c5(342385, 342386, 342387, 342388, 342389, 342390) c6( ) c7(sO, ey, fK, af); #c1(MS4A8) c2(NP_113645) c3(membrane-spanning 4-domains subfamily A member 8) c4(221911) c5(221912, 221913, 221914, 221915, 221916, 221917) c6( ) c7(bE, T, ae, ad, g); #c1(MSANTD3-TMEFF1) c2(NP_001185741) c3(MSANTD3-TMEFF1 fusion protein) c4(76160, 76161, 76162) c5(76163, 76164, 76165, 76166, 76167, 76168, 76169, 76170, 76171, 76172, 76173, 76174) c6( ) c7(dB, T); #c1(MSC) c2(NP_005089) c3(musculin) c4(152798) c5(152799, 152800, 152801, 152802, 152803, 152804) c6( ) c7(ID, T, am, fM, dA, eR, hV, fG, aw, eX, fN, aA, bB, n, aj, bS, v, wV, fd, PJ, aY, V, q, amW, J, eA, ip, x, eW, py, aE, fm, M, ZQ, aT, cy, eV, bI, K, zp, ji, pk, bPQ, N, bA, ly, A, d, aW, Y, oV, xs, hl, r, fe, aF, zC, nr, t, lx, ek, ck, es, lt, ac, c, wg, dB, wO, bE, iA, cj, uJ, m, k, fD, lC, aNI, B); #c1(MSGN1) c2(NP_001099039) c3(mesogenin-1) c4(41764) c5(41765, 41766, 41767, 41768, 41769, 41770) c6( ) c7(fr); #c1(MSH2) c2(NP_000242) c3(DNA mismatch repair protein Msh2 isoform 1) c4(5409, 5410, 5411, 5412, 5413, 5414, 5415) c5(5416, 5417, 5418, 5419, 5420, 5421, 5422) c6( ) c7(ID, T, os, aC, hV, if, al, ajp, bB, ci, oJ, aY, ip, x, afr, aE, Qk, K, hu, eL, cp, Qe, eG, aW, LZ, nY, kt, Ku, g, as, zE, bw, wT, ix, fT, eM, nB, iA, cj, k, m, iu, wu, sw, ht, NT, n, oY, fr, qN, eW, br, Bl, jz, hw, kH, auS, dL, hB, dx, hQ, ax, aq, Hu, YF, cM, Ld, iN, bP, dB, hk, Qu, hL, at, HB, oh, MW, it, B, fM, yk, bmz, cez, ew, aA, hy, acn, Qo, lu, q, eA, nv, eO, aT, cy, ae, ne, qw, avg, Uf, s, NE, Y, ev, r, lo, en, bu, cl, t, Px, Vi, iK, cfZ, ck, QG, iM, Dy, ac, au, gX, fP, anD, ij, bt, Mb, id, aD, Dd, iW, jh, bs, KO, bhL, oZ, hF, ad, BY, av, avp, py, Rq, NS, io, YB, ji, wm, Yi, Yj, d, BN, BO, je, qt, ff, gK, aF, iV, adj, bz, dr, dR, bF, es, cfY, aoR, Ew, bE, afd, lO, adg, y, X, jr); #c1(MSH2) c2(NP_001245210) c3(DNA mismatch repair protein Msh2 isoform 2) c4(92710, 92711) c5(92712, 92713, 92714, 92715, 92716, 92717, 92718, 92719, 92720, 92721, 92722, 92723, 92724, 92725, 92726, 92727, 92728, 92729, 92730) c6( ) c7(ID, T, os, aC, hV, if, al, ajp, bB, ci, oJ, aY, ip, x, afr, aE, Qk, K, hu, eL, cp, Qe, eG, aW, LZ, nY, kt, Ku, g, as, zE, bw, wT, ix, fT, eM, nB, iA, cj, k, m, iu, wu, sw, ht, NT, n, oY, fr, qN, eW, br, Bl, jz, hw, KH, auS, dL, hB, dx, hQ, ax, aq, Hu, YF, cM, Ld, iN, bP, dB, hk, Qu, hL, at, HB, oh, MW, it, B, fM, yk, bmz, cez, ew, aA, hy, acn, do, lu, q, eA, nv, eO, aT, cy, ae, ne, qw, avg, Uf, s, NE, Y, ev, r, lo, en, bu, cl, t, Px, Vi, iK, cfZ, ck, QG, iM, Dy, ac, au, gX, fP, anD, ij, bt, Mb, id, aD, Dd, iW, jh, bs, KO, bhL, oZ, hF, ad, BY, av, avp, py, Rq, NS, io, YB, ji, wm, Yi, Yj, d, BN, BO, je, qt, ff, gK, aF, iV, adj, bz, dr, dR, bF, es, cfY, aoR, Ew, bE, afd, lO, adg, y, X, jr); #c1(MSH2) c2(XP_005264389) c3(DNA mismatch repair protein Msh2 isoform XI) c4(278926) c5(278927, 278928, 278929, 278930, 278931, 278932) c6( ) c7(ID, T, os, aC, hV, if, al, ajp, bB, ci, oJ, aY, ip, x, afr, aE, Qk, K, hu, eL, cp, Qe, eG, aW, LZ, nY, kt, Ku, g, as, zE, bw, wT, ix, fT, eM, nB, iA, cj, k, m, iu, wu, sw, ht, NT, n, oY, fr, qN, eW, br, Bl, jz, hw, KH, auS, dL, hB, dx, hQ, ax, aq, Hu, YF, cM, Ld, iN, bP, dB, hk, Qu, hL, at, HB, oh, MW, it, B, fM, yk, bmz, cez, ew, aA, hy, acn, Qo, lu, q, eA, nv, eO, aT, cy, ae, ne, qw, avg, Uf, s, NE, Y, ev, r, lo, en, bu, cl, t, Px, Vi, iK, cfZ, ck, QG, iM, Dy, ac, au, gX, fP, anD, ij, bt, Mb, id, aD, Dd, iW, jh, bs, KO, bhL, oZ, hF, ad, BY, av, avp, py, Rq, NS, io, YB, ji, wm, Yi, Yj, d, BN, BO, je, qt, ff, gK, aF, iV, adj, bz, dr, dR, bF, es, cfY, aoR, Ew, bE, afd, lO, adg, y, X, jr); #c1(MSH2) c2(XP_011531169) c3(DNA mismatch repair protein Msh2 isoform X2) c4( ) c5(327125, 327126, 327127, 327128, 327129, 327130, 327131, 327132, 327133, 327134, 327135, 327136, 327137, 327138, 327139, 327140, 327141, 327142, 327143, 327144, 327145, 327146, 327147, 327148, 327149, 327150, 327151, 327152) c6( ) c7(ID, T, os, aC, hV, if, al, ajp, bB, ci, oJ, aY, ip, x, afr, aE, Qk, K, hu, eL, cp, Qe, eG, aW, LZ, nY, kt, Ku, g, as, zE, bw, wT, ix, fT, eM, nB, iA, cj, k, m, iu, wu, sw, ht, NT, n, oY, fr, qN, eW, br, Bl, jz, hw, kH, auS, dL, hB, dx, hQ, ax, aq, Hu, YF, cM, Ld, iN, bP, dB, hk, Qu, hL, at, HB, oh, MW, it, B, fM, yk, bmz, cez, ew, aA, hy, acn, Do, lu, q, eA, nv, eO, aT, cy, ae, ne, qw, avg, Uf, s, NE, Y, ev, r, lo, en, bu, cl, t, Px, Vi, iK, cfZ, ck, QG, iM, Dy, ac, au, gX, fP, anD, ij, bt, Mb, id, aD, Dd, iW, jh, bs, KO, bhL, oZ, hF, ad, BY, av, avp, py, Rq, NS, io, YB, ji, wm, Yi, Yj, d, BN, BO, je, qt, ff, gK, aF, iV, adj, bz, dr, dR, bF, es, cfY, aoR, Ew, bE, afd, lO, adg, y, X, jr); #c1(MSH3) c2(NP_002430) c3(DNA mismatch repair protein Msh3) c4(128400, 128401) c5(128402, 128403, 128404, 128405, 128406, 128407, 128408, 128409, 128410, 128411, 128412, 128413) c6( ) c7(T, os, aC, bO, fd, bB, ci, x, aE, K, hu, eL, bc, Qe, aW, Qq, g, as, eF, kJ, ir, cm, bw, eM, nB, m, k, gn, iK, dD, iB, aw, ht, n, fr, ft, kl, kH, h, dL, ia, aq, dB, hL, at, B, aA, q, er, XX, afQ, aT, ae, qw, s, iU, Y, ev, r, cl, t, iK, hY, ck, iM, gX, bt, ao, kG, hS, ha, jh, oZ, eq, hF, iJ, ad, ib, d, je, BO, BH, kD, aF, bz, bE, y, iu); #c1(MSH5) c2(NP_079535) c3 (mutS protein homolog 5 isoform a) c4(218794, 218795, 218796, 218797, 218798, 218799, 218800, 218801) c5(218802, 218803, 218804, 218805, 218806, 218807, 218808, 218809, 218810, 218811, 218812, 218813, 218814, 218815, 218816, 218817, 218818, 218819, 218820, 218821, 218822, 218823, 218824, 218825, 218826, 218827, 218828, 218829, 218830, 218831, 218832, 218833, 218834, 218835, 218836, 218837, 218838, 218839, 218840, 218841, 218842, 218843, 218844, 218845, 218846, 218847, 218848, 218849, 218850, 218851, 218852, 218853, 218854, 218855, 218856, 218857, 218858, 218859, 218860, 218861, 218862, 218863, 218864, 218865, 218866, 218867, 218868, 218869, 218870, 218871, 218872, 218873, 218874) c6( ) c7(T, A, gU, W, ND, iI, iW, lC, eZ, vH, lY, y, Wt); #c1(MSH5) c2(NP_751897) c3(mutS protein homolog 5 isoform b) c4(248238, 248239, 248240, 248241, 248242, 248243, 248244, 248245) c5(248246, 248247, 248248, 248249, 248250, 248251, 248252, 248253, 248254, 248255, 248256, 248257, 248258, 248259, 248260, 248261, 248262, 248263, 248264, 248265, 248266, 248267, 248268, 248269, 248270, 248271, 248272, 248273) c6( ) c7(T, A, gU, W, ND, iI, iW, lC, eZ, vH, lY, y, Wt); #c1(MSH5) c2(NP_751898) c3(mutS protein homolog 5 isoform c) c4(248274, 248275, 248276, 248277, 248278, 248279, 248280, 248281, 248282, 248283, 248284, 248285, 248286, 248287) c5(248288, 248289, 248290, 248291, 248292, 248293, 248294, 248295, 248296, 248297, 248298, 248299, 248300, 248301, 248302, 248303, 248304, 248305, 248306, 248307, 248308, 248309, 248310, 248311, 248312, 248313, 248314, 248315, 248316, 248317, 248318, 248319, 248320, 248321, 248322, 248323, 248324, 248325, 248326, 248327, 248328, 248329, 248330, 248331, 248332, 248333, 248334, 248335, 248336, 248337, 248338, 248339, 248340, 248341, 248342, 248343, 248344, 248345, 248346, 248347, 248348, 248349, 248350, 248351, 248352, 248353, 248354, 248355, 248356, 248357, 248358, 248359, 248360, 248361, 248362, 248363, 248364, 248365, 248366, 248367, 248368, 248369, 248370, 248371, 248372, 248373, 248374, 248375, 248376, 248377, 248378, 248379, 248380, 248381, 248382, 248383, 248384, 248385, 248386, 248387, 248388, 248389, 248390, 248391, 248392, 248393, 248394, 248395, 248396, 248397, 248398, 248399, 248400, 248401, 248402, 248403, 248404, 248405, 248406, 248407, 248408, 248409, 248410, 248411, 248412, 248413, 248414, 248415, 248416, 248417, 248418, 248419, 248420, 248421, 248422, 248423, 248424, 248425, 248426, 248427, 248428, 248429, 248430, 248431, 248432, 248433, 248434, 248435, 248436, 248437, 248438, 248439, 248440, 248441, 248442, 248443, 248444, 248445, 248446, 248447, 248448, 248449, 248450, 248451, 248452, 248453, 248454, 248455, 248456, 248457, 248458, 248459, 248460, 248461, 248462, 248463, 248464, 248465, 248466, 248467, 248468, 248469, 248470, 248471, 248472, 248473, 248474, 248475, 248476, 248477, 248478, 248479, 248480, 248481, 248482, 248483, 248484, 248485, 248486, 248487, 248488, 248489, 248490, 248491, 248492, 248493, 248494, 248495, 248496, 248497, 248498, 248499, 248500, 248501, 248502, 248503, 248504, 248505, 248506, 248507) c6( ) c7(T, A, gU, W, ND, iI, iW, lC, eZ, vH, lY, y, Wt); #c1(MSH6) c2(NP_000170) c3(DNA mismatch repair protein Msh6 isoform 1) c4(3767, 3768, 3769) c5(3770, 3771, 3772, 3773, 3774, 3775, 3776, 3777, 3778, 3779, 3780, 3781, 3782, 3783, 3784, 3785, 3786, 3787, 3788) c6( ) c7(gV, T, os, aC, id, lT, iW, NT, jh, aA, bB, n, oZ, OE, oH, ci, bX, fr, q, ad, eA, dP, av, afr, b, iu, XX, eO, kH, aT, auS, dL, ey, ae, eL, dx, cp, Qe, jf, gj, yo, d, je, BO, s, hG, aW, Y, PL, vT, ev, NE, r, Kf, aF, g, cl, as, t, adj, aHd, bF, iM, Ew, dB, bE, gX, hL, at, SZ, m, MC, i, y, X, cga); #c1(MSH6) c2(NP_001268421) c3(DNA mismatch repair protein Msh6 isoform 2) c4(99879, 99880, 99881) c5(99882, 99883, 99884, 99885, 99886, 99887, 99888, 99889, 99890, 99891, 99892, 99893) c6( ) c7(gV, T, os, aC, id, lT, iW, NT, jh, aA, bB, n, oZ, OE, oH, ci, bX, fr, q, ad, eA, dP, av, afr, b, iu, XX, eO, kH, aT, auS, dL, ey, ae, eL, dx, cp, Qe, jf, gj, yo, d, je, BO, s, hG, aW, Y, PL, vT, ev, NE, r, Kf, aF, g, cl, as, t, adj, aHd, bF, iM, Ew, dB, bE, gX, hL, at, SZ, m, MC, i, y, X, cga); #c1(MSH6) c2(NP_001268423) c3(DNA mismatch repair protein Msh6 isoform 3) c4( ) c5(99894, 99895, 99896, 99897, 99898) c6( ) c7(gV, T, os, aC, id, lT, iW, NT, jh, aA, bB, n, oZ, OE, oH, ci, bX, fr, q, ad, eA, dP, av, afr, b, iu, XX, eO, kH, aT, auS, dL, ey, ae, eL, dx, cp, Qe, jf, gj, yo, d, je, BO, s, hG, aW, Y, PL, vT, ev, NE, r, Kf, aF, g, cl, as, t, adj, aHd, bF, iM, Ew, dB, bE, gX, hL, at, SZ, m, MC, i, y, X, cga); #c1(MSI1) c2(NP_002433) c3(RNA-binding protein Musashi homolog 1) c4(128414) c5(128415, 128416, 128417, 128418, 128419, 128420) c6( ) c7(T, fR, aA, bB, ci, qN, q, eA, eW, dP, gd, aT, dL, K, eL, af, eu, jf, d, s, aW, ev, ff, r, aq, aF, en, cl, g, bz, bb, kP, wg, ig, dB, bE, hL, at, B); #c1(MSI2) c2(NP_620412) c3(RNA-binding protein Musashi homolog 2 isoform a) c4(237593, 237594) c5(237595, 237596, 237597, 237598, 237599, 237600, 237601, 237602, 237603, 237604, 237605, 237606) c6( ) c7(ck, x, iT, iU, bK, k, bw, bt, bz, B); #c1(MSI2) c2(NP_733839) c3(RNA-binding protein Musashi homolog 2 isoform b) c4(247226) c5(247227, 247228, 247229, 247230, 247231, 247232) c6( ) c7(ck, x, iT, iU, bK, k, bw, bt, bz, B); #c1(MSI2) c2(XP_005257071) c3(RNA-binding protein Musashi homolog 2 isoform X1) c4(274654) c5(274655, 274656, 274657, 274658, 274659, 274660) c6( ) c7(ck, x, iT, iU, bK, k, bw, bt, bz, B); #c1(MSLN) c2(XP_005255091) c3(mesothelin isoform X4) c4(273536) c5(273537, 273538, 273539, 273540, 273541, 273542, 273543, 273544, 273545, 273546, 273547, 273548) c6( ) c7(T, aC, id, sw, YM, aA, bB, n, ci, eJ, ip, x, jz, ib, aE, lt, aT, fb, xQ, no, Jl, aW, ff, nY, agF, kp, en, cl, g, t, bz, kP, bE, m, k, bt, iu, BA, hS); #c1(MSLN) c2(XP_006720900) c3(mesothelin isoform X1) c4(293763, 293764) c5(293765, 293766, 293767, 293768, 293769, 293770, 293771, 293772, 293773, 293774, 293775, 293776, 293777, 293778) c6( ) c7(T, aC, id, sw, YM, aA, bB, n, ci, eJ, ip, x, jz, ib, aE, lt, aT, fb, xQ, nO, Jl, aW, ff, nY, agF, kp, en, cl, g, t, bz, kP, bE, m, k, bt, iu, BA, hS); #c1(MSLN) c2(XP_011520652) c3(mesothelin isoform X2) c4(315543) c5(315544, 315545, 315546, 315547, 315548, 315549, 315550, 315551, 315552, 315553) c6( ) c7(T, aC, id, sw, YM, aA, bB, n, ci, eJ, ip, x, jz, ib, aE, lt, aT, fb, xQ, nO, Jl, aW, ff, nY, agF, kp, en, cl, g, t, bz, kP, bE, m, k, bt, iu, BA, hS); #c1(MSMB) c2(NP_002434) c3(beta-microseminoprotein isoform a precursor) c4(128421) c5(128422, 128423, 128424, 128425, 128426, 128427) c6( ) c7(T, qo, Uc, OI, if, iW, aA, it, n, fr, ci, qN, PJ, q, ad, rc, dP, ft, aE, lg, acu, jZ, pX, dV, aT, bOu, NM, ae, VF, cgb, d, Ub, aW, Y, r, lo, kp, xH, aF, g, cl, kk, iK, bz, op, ar, pu, qe, c, eM, dc, vA, agl, DC, hL, uz, wR, act, ER, eg, Pr, B); #c1(MSMB) c2(NP_619540) c3(beta-microseminoprotein isoform b precursor) c4(236874) c5(236875, 236876, 236877, 236878, 236879, 236880) c6( ) c7(T, qo, Uc, OI, if, iW, aA, it, n, fr, ci, qN, PJ, q, ad, rc, dP, ft, aE, lg, acu, jZ, pX, dV, aT, bOu, NM, ae, VF, cgb, d, Ub, aW, Y, r, lo, kp, xH, aF, g, cl, kk, iK, bz, op, ar, pu, qe, c, eM, dc, vA, agl, DC, hL, uz, wR, act, ER, eg, Pr, B); #c1(MSMO1) c2(NP_001017369) c3(methylsterol monooxygenase 1 isoform 2) c4( ) c5(26341, 26342, 26343, 26344, 26345) c6( ) c7(bL, T, cgc); #c1(MSMO1) c2(NP_006736) c3(methylsterol monooxygenase 1 isoform 1) c4(167591, 167592, 167593, 167594) c5(167595, 167596, 167597, 167598, 167599, 167600, 167601, 167602, 167603, 167604, 167605, 167606, 167607, 167608, 167609, 167610, 167611, 167612, 167613) c6( ) c7(bL, T, cgc); #c1(MSMP) c2(NP_001037729) c3(prostate-associated microseminoprotein precursor) c4(34117) c5(34118, 34119, 34120, 34121, 34122, 34123, 34124, 34125) c6(34126, 34127) c7(ae, ac, ad); #c1(MSN) c2(NP_002435) c3(moesin) c4(128428) c5(128429, 128430, 128431, 128432, 128433, 128434) c6( ) c7(T, aC, fR, fG, if, bB, j, BF, qN, PB, eA, ad, ip, aE, bW, aT, h, By, Fx, ae, d, iE, aW, Y, Ls, ff, lo, en, cl, g, t, iK, rR, Ew, W, bE, AM, hL, dW, oX, gn, m, iu); #c1(MSR1) c2(NP_002436) c3(macrophage scavenger receptor types I and II isoform type 2) c4(128435, 128436) c5(128437, 128438, 128439, 128440, 128441, 128442, 128443, 128444, 128445, 128446, 128447, 128448, 128449, 128450, 128451, 128452, 128453, 128454, 128455, 128456, 128457, 128458, 128459) c6(128460) c7(bL, T, EM, dD, eR, pf, ea, fG, e, n, j, eq, eA, ad, x, aT, dL, ey, XZ, ae, af, RT, cgd, Ub, iU, aW, iZ, LZ, aq, t, bz, LB, ck, at, mf, m, k, it, X); #c1(MSR1) c2(NP_619729) c3(macrophage scavenger receptor types I and II isoform type 1) c4(237071, 237072, 237073) c5(237074, 237075, 237076, 237077, 237078, 237079, 237080, 237081, 237082, 237083, 237084, 237085, 237086, 237087, 237088, 237089) c6( ) c7(bL, T, EM, dD, eR, pf, ea, fG, e, n, j, eq, eA, ad, x, aT, dL, ey, XZ, ae, af, RT, cgd, Ub, iU, aW, iZ, LZ, aq, t, bz, LB, ck, at, mf, m, k, it, X); #c1(MSR1) c2(NP_619730) c3(macrophage scavenger receptor types I and II isoform type 3) c4(237090) c5(237091, 237092, 237093, 237094, 237095, 237096, 237097, 237098, 237099, 237100, 237101, 237102, 237103, 237104) c6( ) c7(bL, T, EM, dD, eR, pf, ea, fG, e, n, j, eq, eA, ad, x, aT, dL, ey, XZ, ae, af, RT, cgd, Ub, iU, aW, iZ, LZ, aq, t, bz, LB, ck, at, mf, m, k, it, X); #c1(MSRA) c2(NP_001129142) c3(mitochondrial peptide methionine sulfoxide reductase isoform b) c4(50895) c5(50896, 50897, 50898, 50899, 50900, 50901) c6( ) c7(y, ac, hX, e, aw, qQ, U, cz, v, j, K, qj, yC, cm, IB, B, ao, pC); #c1(MSRA) c2(NP_001129143) c3(mitochondrial peptide methionine sulfoxide reductase isoform c) c4(50902) c5(50903, 50904, 50905, 50906, 50907, 50908, 50909) c6( ) c7(y, ac, hX, e, aw, qQ, U, cz, v, j, K, qj, yC, cm, lB, B, ao, pC); #c1(MSRA) c2(NP_001186658) c3(mitochondrial peptide methionine sulfoxide reductase isoform d) c4( ) c5(78303, 78304, 78305, 78306, 78307) c6( ) c7(y, ac, hX, e, aw, qQ, U, cz, v, j, K, qj, yC, cm, lB, B, ao, pC); #c1(MSRA) c2(NP_036463) c3(mitochondrial peptide methionine sulfoxide reductase isoform a precursor) c4(174047, 174048) c5(174049, 174050, 174051, 174052, 174053, 174054) c6( ) c7(y, ac, hX, e, aw, qQ, U, cz, v, j, K, qj, yC, cm, lB, B, ao, pC); #c1(MSRB2) c2(NP_036360) c3(methionine-R-sulfoxide reductase B2, mitochondrial precursor) c4(173219) c5(173220, 173221, 173222, 173223, 173224, 173225, 173226) c6( ) c7(qj); #c1(MSRB3) c2(NP_001180390) c3(methionine-R-sulfoxide reductase B3 isoform 2 precursor) c4(73914, 73915, 73916) c5(73917, 73918, 73919, 73920, 73921, 73922, 73923, 73924, 73925, 73926, 73927, 73928, 73929, 73930, 73931, 73932, 73933, 73934, 73935, 73936, 73937, 73938) c6( ) c7(bK, aHs, cgf, cge, EQ); #c1(MSRB3) c2(NP_932346) c3(methionine-R-sulfoxide reductase B3 isoform 1 precursor) c4(258966) c5(258967, 258968, 258969, 258970, 258971, 258972) c6( ) c7(bk, aHs, cgf, cge, EQ); #c1(MST1) c2(NP_066278) c3(hepatocyte growth factor-like protein precursor) c4(206938, 206939, 206940) c5(206941, 206942, 206943, 206944, 206945, 206946, 206947, 206948, 206949, 206950, 206951, 206952, 206953) c6( ) c7(T, qo, Uc, ra, OI, bgQ, fG, if, aw, aA, bB, it, fr, ci, qN, PJ, V, q, ad, rc, eA, dP, av, ft, hd, aE, lg, jz, acu, cc, bW, jZ, pX, dV, aT, K, Tj, bOu, NM, ae, VF, uQ, d, aW, Y, ff, r, Pr, hN, lo, aF, xH, fe, en, cl, kk, jT, g, bz, iK, op, iV, LB, nM, qe, c, nB, bE, dc, eM, vA, agl, DC, mx, hL, apv, uz, eo, ER, eg, zA, B); #c1(MST1) c2(XP_011532038) c3(hepatocyte growth factor-like protein isoform X8) c4(328097) c5( ) c6( ) c7(T, qo, Uc, ra, OI, bgQ, fG, if, aw, aA, bB, it, fr, ci, qN, PJ, V, q, ad, rc, eA, dP, av, ft, hd, aE, lg, jz, acu, cc, bW, jZ, pX, dV, aT, K, Tj, bOu, NM, ae, VF, uQ, d, aW, Y, ff, r, Pr, hN, lo, aF, xH, fe, en, cl, kk, jT, g, bz, iK, op, iV, LB, nM, qe, c, nB, bE, dc, eM, vA, agl, DC, mx, hL, apv, uz, eo, ER, eg, zA, B); #c1(MST1) c2(XP_011532040) c3(hepatocyte growth factor-like protein isoform X11) c4( ) c5(328098, 328099, 328100, 328101, 328102, 328103, 328104, 328105, 328106, 328107, 328108) c6( ) c7(T, qo, Uc, ra, OI, bgQ, fG, if, aw, aA, bB, it, fr, ci, qN, PJ, V, q, ad, rc, eA, dP, av, ft, hd, aE, lg, jz, acu, cc, bW, jZ, pX, dV, aT, K, Tj, bOu, NM, ae, VF, uQ, d, aW, Y, ff, r, Pr, hN, lo, aF, xH, fe, en, cl, kk, jT, g, bz, iK, op, iV, LB, nM, qe, c, nB, bE, dc, eM, vA, agl, DC, mx, hL, apv, uz, eo, ER, eg, zA, B); #c1(MST1R) c2(NP_001231866) c3(macrophage-stimulating protein receptor isoform 2 precursor) c4(87578) c5(87579, 87580, 87581, 87582, 87583, 87584) c6( ) c7(T, WA, rQ, jh, aA, bB, BH, fr, ci, V, q, aR, ad, eA, dP, av, ft, hd, aE, lg, pX, akJ, aT, h, dL, pk, ae, eL, B, s, aW, ff, r, fe, lo, aF, en, aDY, g, LV, t, bz, eF, iV, ck, eM, nB, bE, m, X); #c1(MST1R) c2(NP_002438) c3(macrophage-stimulating protein receptor isoform 1 preproprotein) c4(128468) c5(128469, 128470, 128471, 128472, 128473, 128474) c6( ) c7(T, WA, rQ, jh, aA, bB, BH, fr, ci, V, q, aR, ad, eA, dP, av, ft, hd, aE, lg, pX, akJ, aT, h, dL, pk, ae, eL, B, s, aW, ff, r, fe, lo, aF, en, aDY, g, LV, t, bz, eF, iV, ck, eM, nB, bE, m, X); #c1(MSTN) c2(NP_005250) c3(growth/differentiation factor 8 precursor) c4(154394) c5(154395, 154396, 154397, 154398, 154399, 154400) c6( ) c7(bL, T, EM, ea, cgg, e, vX, wo, tc, bS, v, ct, Eh, q, cd, bv, Yc, oz, Ae, oj, kf, h, bgr, bl, K, nJ, DA, xs, fc, r, TQ, dq, uY, aaO, Z, fq, op, c, bQl, vA, U, gZ, at, cP, gT); #c1(MSTO1) c2(NP_001243462) c3(protein misato homolog 1 isoform c) c4( ) c5(90874, 90875, 90876, 90877, 90878) c6( ) c7(x, W, eM, aW, aT, k, pd, hq); #c1(MSTO1) c2(NP_060586) c3(protein misato homolog 1 isoform a) c4(196683, 196684) c5(196685, 196686, 196687, 196688, 196689, 196690, 196691) c6( ) c7(x, W, eM, aW, aT, k, pd, hq); #c1(MSTO1) c2(XP_011507998) c3(protein misato homolog 1 isoform X2) c4(298436) c5(298437, 298438, 298439, 298440, 298441, 298442) c6( ) c7(x, W, eM, aW, aT, k, pd, hq); #c1(MSTO1) c2(XP_011508004) c3(protein misato homolog 1 isoform X8) c4(298443) c5( ) c6( ) c7(x, W, eM, aW, aT, k, pd, hq); #c1(MSTO1) c2(XP_011508011) c3(protein misato homolog 1 isoform X13) c4( ) c5(298444) c6( ) c7(x, W, eM, aW, aT, k, pd, hq); #c1(MSTO1) c2(XP_011508012) c3(protein misato homolog 1 isoform X14) c4( ) c5(298445, 298446, 298447, 298448, 298449, 298450, 298451, 298452, 298453, 298454) c6( ) c7(x, W, eM, aW, aT, k, pd, hq); #c1(MSX1) c2(NP_002439) c3(homeobox protein MSX-1) c4(128475) c5(128476, 128477, 128478, 128479, 128480, 128481) c6( ) c7(Tb, if, bjB, eX, aA, GC, fd, cgk, aml, qN, cgj, boJ, amH, cgl, Na, axC, amJ, aT, eV, rD, Nc, an, rz, cgi, yi, aoa, bjy, aW, Az, cm, apM, rR, cgh, bpv, aen); #c1(MSX2) c2(NP_002440) c3(homeobox protein MSX-2) c4(128482) c5(128483, 128484, 128485, 128486, 128487, 128488, 128489) c6( ) c7(T, aC, aw, al, cz, n, TZ, aml, C, Na, aE, aT, cy, Nc, apw, Ur, aOX, cgi, uX, mR, TY, aW, Y, Az, akB, kp, cgm, agj, t, cgo, zV, gk, apM, cgn, c, cgp, anD, m, art); #c1(MT1A) c2(NP_005937) c3(metallothionein-(A) c4(160540) c5(160541, 160542, 160543, 160544, 160545, 160546, 160547, 160548, 160549, 160550, 160551, 160552) c6( ) c7(bL, G, T, fR, FG, sw, iW, e, aA, n, aj, Vd, ci, uE, PJ, ml, fr, J, ad, Vp, hd, lg, oz, nv, Qg, aT, dL, yh, ae, B, s, dH, aW, ff, eZ, fQ, aF, en, dr, dS, nM, eM, nB, QF, Km, Pk, X); #c1(MT1B) c2(NP_005938) c3(metallothionein-1B) c4(160553) c5(160554, 160555, 160556, 160557, 160558, 160559, 160560) c6( ) c7(bL, T, ZV, FG, iW, e, aj, j, PJ, J, ec, aT, ZU, jy, K, B, dH, aW, g, fi, dS, eM, Km, ei, gT, X); #c1(MT1E) c2(NP_783316) c3(metallothionein-1E) c4(251609) c5(251610, 251611, 251612, 251613, 251614, 251615, 251616, 251617, 251618, 251619, 251620) c6( ) c7(bL, T, FG, iW, ha, bS, Vd, fr, PJ, eA, aT, dL, eL, aa, B, dH, qt, aW, cl, dS, aQn, ig, eM, hL, Km, X); #c1(MT1E) c2(XP_005256013) c3(metallothionein-1E isoform X1) c4(273911, 273912) c5(273913, 273914, 273915, 273916, 273917, 273918) c6( ) c7(bL, T, FG, iW, ha, bS, Vd, fr, PJ, eA, aT, dL, eL, aa, B, dH, qt, aW, cl, dS, aQn, ig, eM, hL, Km, X); #c1(MT1F) c2(NP_005940) c3(metallothionein-IF isoform 1) c4(160561) c5(160562, 160563, 160564, 160565, 160566, 160567) c6( ) c7(bL, iO, T, Ve, no, eM, dH, FG, aW, qt, iW, X, an, aT, aq, Km, dy, PJ, B, dS); #c1(MT1G) c2(NP_001288196) c3(metallothionein-1G isoform 2) c4(113694, 113695) c5(113696, 113697, 113698, 113699, 113700, 113701, 113702, 113703, 113704, 113705, 113706, 113707) c6( ) c7(bL, FG, iW, ht, fr, PJ, q, ad, hd, aT, K, ae, bA, B, dH, r, dS, iO, nM, eM, nB, Km, bt, X); #c1(MT1G) c2(NP_005941) c3(metallothionein-1G isoform 1) c4(160568) c5(160569, 160570, 160571, 160572, 160573, 160574) c6( ) c7(bL, FG, iW, ht, fr, PJ, q, ad, hd, aT, K, ae, bA, B, dH, r, dS, iO, nM, eM, nB, Km, bt, X); #c1(MT1H) c2(NP_005942) c3(metallothionein-1H) c4(160575) c5( ) c6( ) c7(bL, T, eM, dH, FG, aW, iW, X, aT, aq, dL, Km, K, fr, PJ, iR, eL, B, dS); #c1(MT1M) c2(NP_789846) c3(metallothionein-1M) c4(252401) c5(252402, 252403, 252404, 252405, 252406, 252407) c6( ) c7(bL, eM, dH, FG, iW, X, aT, Km, K, PJ, B, dS); #c1(MT1X) c2(NP_005943) c3(metallothionein-1X) c4(160576) c5(160577, 160578, 160579, 160580, 160581, 160582) c6( ) c7(bL, eY, B, eM, dH, FG, eL, iW, ff, aT, dL, Km, en, PJ, ae, X, ad, dS); #c1(MT2A) c2(NP_005944) c3(metallothionein-2) c4(160583, 160584) c5(160585, 160586, 160587, 160588, 160589, 160590, 160591, 160592, 160593, 160594, 160595, 160596) c6( ) c7(bL, G, Ve, T, dD, FG, ea, bmk, ht, jh, aA, bB, vr, bS, dG, ct, uE, Gn, v, eq, ad, no, yt, aT, dL, ji, K, jo, rz, ae, eL, aiB, aMG, aW, eH, eZ, aF, Z, t, nC, eF, ac, bE, gZ, QF, dy, m, rA, B); #c1(MT3) c2(NP_005945) c3(metallothionein-3) c4(160597) c5(160598, 160599, 160600, 160601, 160602, 160603) c6( ) c7(G, T, wi, OI, bS, ct, ci, ad, BI, aT, Lv, SH, dL, H, eL, s, EV, ff, vs, aF, en, g, Z, hq, ck, dS, eM, mz, my, gT, bt, L); #c1(MT4) c2(NP_116324) c3(metallothionein-4) c4(226158) c5(226159, 226160, 226161, 226162, 226163, 226164) c6( ) c7(eM); #c1(MTA1) c2(NP_001190187) c3(metastasis-associated protein MTA1 isoform MTA1s) c4(80333) c5(80334, 80335, 80336, 80337, 80338) c6( ) c7(T, fM, zX, jh, aA, lD, n, ci, pd, q, ad, hX, dP, aE, rJ, pX, aT, K, hu, fb, ae, Yi, asp, qt, aW, Y, anx, ff, aFt, r, aq, en, cl, g, t, cM, bz, iR, W, eM, AM, m, im, it, B); #c1(MTA1) c2(NP_004680) c3(metastasis-associated protein MTA1 isoform MTA1) c4(148799) c5(148800, 148801, 148802, 148803, 148804, 148805) c6( ) c7(T, fM, zX, jh, aA, lD, n, ci, pd, q, ad, hX, dP, aE, rJ, pX, aT, K, hu, fb, ae, Yi, asp, qt, aW, Y, anx, ff, aFt, r, aq, en, cl, g, t, cM, bz, iR, W, eM, AM, m, im, it, B); #c1(MTA1) c2(XP_011535613) c3(metastasis-associated protein MTA1 isoform X11) c4( ) c5(332859, 332860) c6( ) c7(T, fM, zX, jh, aA, lD, n, ci, pd, q, ad, hX, dP, aE, rJ, pX, aT, K, hu, fb, ae, Yi, asp, qt, aW, Y, anx, ff, aFt, r, aq, en, cl, g, t, cM, bz, iR, W, eM, AM, m, im, it, B); #c1(MTA1) c2(XP_011535614) c3(metastasis-associated protein MTA1 isoform X12) c4(332861, 332862) c5( ) c6( ) c7(T, fM, zX, jh, aA, lD, n, ci, pd, q, ad, hX, dP, aE, rJ, pX, aT, K, hu, fb, ae, Yi, asp, qt, aW, Y, anx, ff, aFt, r, aq, en, cl, g, t, cM, bz, iR, W, eM, AM, m, im, it, B); #c1(MTA1) c2(XP_011535615) c3(metastasis-associated protein MTA1 isoform X13) c4( ) c5(332863, 332864, 332865, 332866, 332867, 332868, 332869, 332870, 332871, 332872, 332873) c6( ) c7(T, fM, zX, jh, aA, lD, n, ci, pd, q, ad, hX, dP, aE, rJ, pX, aT, K, hu, fb, ae, Yi, asp, qt, aW, Y, anx, ff, aFt, r, aq, en, cl, g, t, cM, bz, iR, W, eM, AM, m, im, it, B); #c1(MTA1) c2(XP_011535617) c3(metastasis-associated protein MTA1 isoform X15) c4( ) c5(332874) c6( ) c7(T, fM, zX, jh, aA, lD, n, ci, pd, q, ad, hX, dP, aE, rJ, pX, aT, K, hu, fb, ae, Yi, asp, qt, aW, Y, anx, ff, aFt, r, aq, en, cl, g, t, cM, bz, iR, W, eM, AM, m, im, it, B); #c1(MTA2) c2(NP_004730) c3(metastasis-associated protein MTA2) c4(149130, 149131) c5(149132, 149133, 149134, 149135, 149136, 149137, 149138, 149139, 149140, 149141, 149142, 149143, 149144, 149145, 149146, 149147) c6( ) c7(T, dA, aA, bB, n, Vs, dP, aT, dL, K, eL, aW, cgq, eZ, aFr, kp, aF, BG, bz, eM, bE, eB, zA); #c1(MTA3) c2(NP_065795) c3(metastasis-associated protein MTA3 isoform b) c4(205480) c5(205481, 205482, 205483, 205484, 205485, 205486) c6( ) c7(T, fM, eM, aW, jh, aT, aj, m, g, t, bz); #c1(MTA3) c2(XP_005264513) c3(metastasis-associated protein MTA3 isoform X1) c4(278976, 278977) c5(278978, 278979, 278980, 278981, 278982, 278983, 278984, 278985, 278986, 278987, 278988, 278989) c6( ) c7(T, fM, eM, aW, jh, aT, aj, m, g, t, bz); #c1(MTAP) c2(NP_002442) c3(S-methyl-5′-thioadenosine phosphorylase) c4(128490) c5(128491, 128492, 128493, 128494, 128495, 128496) c6( ) c7(bL, sc, T, kh, cgs, aC, anl, pf, aw, jh, aA, cz, aj, ci, eA, ad, eJ, x, ib, aE, pX, rA, kH, aT, K, dQ, fb, ae, Zm, eu, je, s, anv, Jl, aW, Y, MQ, eH, nO, ff, nY, Ej, aF, en, g, eF, bz, ck, iM, Dy, eM, bP, bE, hk, azT, cgr, k, bt, ik, B); #c1(MTBP) c2(NP_071328) c3(mdm2-binding protein) c4(211001) c5(211002, 211003, 211004, 211005, 211006, 211007) c6( ) c7(T, fM, aW, eH, aT, j, eF); #c1(MTCH1) c2(NP_001258570) c3(mitochondrial carrier homolog 1 PSAP-LL) c4(96146, 96147) c5(96148, 96149, 96150, 96151, 96152, 96153, 96154, 96155, 96156, 96157, 96158, 96159) c6( ) c7(AK, vU, T, eG, afu, vi); #c1(MTCH1) c2(NP_055156) c3(mitochondrial carrier homolog 1 PSAP-LS) c4(179491, 179492, 179493) c5(179494, 179495, 179496, 179497, 179498, 179499, 179500, 179501, 179502, 179503, 179504, 179505, 179506, 179507, 179508, 179509, 179510, 179511, 179512) c6( ) c7(AK, vU, T, eG, afu, vi); #c1(MTCH1) c2(XP_005249036) c3(mitochondrial carrier homolog 1 isoform X2) c4(268872, 268873) c5(268874, 268875, 268876, 268877, 268878, 268879, 268880) c6( ) c7(AK, vU, T, eG, afu, vi); #c1(MTCH1) c2(XP_011512752) c3(mitochondrial carrier homolog 1 isoform X3) c4(303770) c5(303771, 303772, 303773, 303774, 303775, 303776, 303777, 303778, 303779, 303780) c6( ) c7(AK, vU, T, eG, afu, vi); #c1(MTCH2) c2(NP_055157) c3(mitochondrial carrier homolog 2) c4(179513) c5(179514, 179515, 179516, 179517, 179518) c6( ) c7(bL, bM, T, e, aq, v, fq); #c1(MTCL1) c2(NP_056025) c3(microtubule cross-linking factor 1) c4(185215) c5(185216, 185217, 185218, 185219, 185220) c6( ) c7(ht, ff, cd, ac, en); #c1(MTCL1) c2(XP_005258155) c3 (microtubule cross-linking factor 1 isoform X2) c4( ) c5(275527) c6( ) c7(ht, ff, cd, ac, en); #c1(MTCL1) c2(XP_005258156) c3(microtubule cross-linking factor 1 isoform X4) c4(275528) c5(275529, 275530, 275531, 275532, 275533, 275534) c6( ) c7(ht, ff, cd, ac, en); #c1(MTCL1) c2(XP_011523942) c3(microtubule cross-linking factor 1 isoform X1) c4(318484) c5(318485, 318486, 318487, 318488, 318489, 318490) c6( ) c7(ht, ff, cd, ac, en); #c1(MTCL1) c2(XP_011523943) c3(microtubule cross-linking factor 1 isoform X3) c4( ) c5(318491) c6( ) c7(ht, ff, cd, ac, en); #c1(MTCP1) c2(NP_001018025) c3(protein p13 MTCP-1) c4(26887) c5(26888, 26889, 26890, 26891, 26892, 26893, 26894, 26895, 26896, 26897, 26898, 26899, 26900) c6( ) c7(ck, fM, Kk, gh, kH, atf, aKO, ho, ad); #c1(MTDH) c2(NP_848927) c3 (protein LYR1C) c4(254223) c5(254224, 254225, 254226, 254227, 254228, 254229) c6( ) c7(G, T, if, ht, jx, NT, aA, bB, n, uu, ci, qN, q, aR, ad, hX, eA, ke, aT, h, DO, K, hu, ae, d, aW, Y, ff, eH, r, aq, Ej, aF, agF, en, cl, eF, t, apx, cm, iK, iR, kP, eM, dB, bE, U, hL, m, qF, bt, it, B); #c1(MTERF1) c2(XP_006716189) c3(transcription termination factor 1, mitochondrial isoform X1) c4(290151, 290152) c5(290153, 290154, 290155, 290156, 290157, 290158, 290159, 290160, 290161, 290162, 290163, 290164) c6( ) c7(awN, kC, m); #c1(MTF1) c2(XP_011539794) c3(metal regulatory transcription factor 1 isoform X1) c4(338002, 338003) c5(338004, 338005, 338006, 338007, 338008, 338009, 338010) c6( ) c7(nM, Db, nB, gT, B, bN); #c1(MTF1) c2(XP_011539795) c3(metal regulatory transcription factor 1 isoform X2) c4( ) c5(338011, 338012, 338013, 338014, 338015, 338016) c6( ) c7(nM, Db, nB, gT, B, bN); #c1(MTFMT) c2(NP_640335) c3(methionyl-tRNA formyltransferase, mitochondrial) c4(238425) c5(238426, 238427, 238428, 238429, 238430, 238431, 238432, 238433, 238434, 238435) c6( ) c7(T, ajU, cgt, bxl, bZ, ae, ad); #c1(MTFP1) c2(NP_001003704) c3(mitochondrial fission process protein 1 isoform b) c4(20001) c5(20002, 20003, 20004, 20005, 20006, 20007) c6( ) c7(eR); #c1(MTFP1) c2(NP_057582) c3(mitochondrial fission process protein 1 isoform a) c4(191470) c5(191471, 191472, 191473, 191474, 191475, 191476) c6( ) c7(eR); #c1(MTG1) c2(NP_612393) c3(mitochondrial ribosome-associated GTPase 1) c4(236342, 236343, 236344) c5(236345, 236346, 236347, 236348, 236349, 236350, 236351, 236352, 236353, 236354, 236355, 236356, 236357, 236358, 236359, 236360, 236361, 236362) c6( ) c7(T, jv, lV, ha, bAp, bO, cz, cR, n, aj, dG, zh, bB, qN, qy, kv, q, J, cbh, ad, ai, eA, av, b, aji, xE, ft, hd, aWc, sO, aE, aeO, aT, aU, DO, jy, iY, K, ae, yo, B, aeR, wr, Wi, Y, aW, Ls, ff, eZ, r, Mz, gl, aq, Hu, en, Tp, cm, boD, bz, dr, qa, EJ, bF, es, byl, Ew, jx, CA, iA, bE, bP, bo, cj, mf, i, bt, bAs, cgu, ao); #c1(MTHFD1) c2(NP_005947) c3(C-1-tetrahydrofolate synthase, cytoplasmic) c4(160604, 160605) c5(160606, 160607, 160608, 160609, 160610, 160611, 160612, 160613, 160614, 160615, 160616, 160617) c6( ) c7(ng, T, jv, dD, fR, sQ, arQ, aA, bB, v, uu, eq, q, ad, x, av, Na, ft, fS, MP, aT, Nc, bK, lQ, an, ae, af, eu, bf, hG, aW, caD, r, Ll, Lk, g, Z, t, uC, bz, f, bN, ck, sn, ac, eM, lp, bE, ag, wE, bm, uo, at, ut, k, m, bD, y); #c1(MTHFD1L) c2(NP_001229696) c3(monofunctional C1-tetrahydrofolate synthase, mitochondrial isoform 1 precursor) c4(84826) c5( ) c6( ) c7(A, ac, af, aw, j, f, fq, fK, Gc); #c1(MTHFD1L) c2(NP_001229697) c3(monofunctional C1-tetrahydrofolate synthase, mitochondrial isoform 3) c4(84827) c5( ) c6( ) c7(A, ac, af, aw, j, f, fq, fK, Gc); #c1(MTHFD1L) c2(NP_001229698) c3(monofunctional C1-tetrahydrofolate synthase, mitochondrial isoform 4 precursor) c4(84828, 84829, 84830) c5(84831, 84832, 84833, 84834, 84835, 84836, 84837, 84838, 84839, 84840, 84841, 84842, 84843, 84844, 84845, 84846, 84847, 84848, 84849, 84850, 84851, 84852, 84853, 84854) c6( ) c7(A, ac, af, aw, j, f, fq, fK, Gc); #c1(MTHFD1L) c2(NP_056255) c3(monofunctional C1-tetrahydrofolate synthase, mitochondrial isoform 2 precursor) c4(186764, 186765) c5(186766, 186767, 186768, 186769, 186770, 186771) c6( ) c7(A, ac, af, aw, j, f, fq, fK, Gc); #c1(MTHFD1L) c2(XP_005266967) c3(monofunctional C1-tetrahydrofolate synthase, mitochondrial isoform X1) c4( ) c5(280547, 280548, 280549, 280550, 280551) c6( ) c7(A, ac, af, aw, j, f, fq, fK, Gc); #c1(MTHFD1L) c2(XP_005266968) c3(monofunctional C1-tetrahydrofolate synthase, mitochondrial isoform X12) c4( ) c5(280552, 280553, 280554, 280555, 280556, 280557, 280558, 280559, 280560, 280561, 280562, 280563, 280564, 280565, 280566, 280567, 280568, 280569, 280570) c6( ) c7(A, ac, af, aw, j, f, fq, fK, Gc); #c1(MTHFD1L) c2(XP_011534033) c3(monofunctional C1-tetrahydrofolate synthase, mitochondrial isoform X5) c4(330207, 330208) c5( ) c6( ) c7(A, ac, af, aw, j, f, fq, fK, Gc); #c1(MTHFD1L) c2(XP_011534036) c3 (monofunctional C1-tetrahydrofolate synthase, mitochondrial isoform X7) c4(330209, 330210) c5(330211, 330212, 330213, 330214, 330215, 330216) c6( ) c7(A, ac, af, aw, j, f, fq, fK, Gc); #c1(MTHFD2) c2(NP_006627) c3(bifunctional methylenetetrahydrofolate dehydrogenase/cyclohydrolase, mitochondrial precursor) c4(166713, 166714, 166715) c5(166716, 166717, 166718, 166719, 166720, 166721, 166722) c6( ) c7(Na, T, lp, aW, aT, at, Nc, dL, bD, B); #c1(MTHFD2) c2(XP_006711987) c3(bifunctional methylenetetrahydrofolate dehydrogenase/cyclohydrolase, mitochondrial isoform X1) c4( ) c5(286313, 286314, 286315, 286316, 286317, 286318, 286319, 286320, 286321, 286322, 286323) c6( ) c7(Na, T, lp, aW, aT, at, Nc, dL, bD, B); #c1(MTHFR) c2(XP_011539797) c3(methylenetetrahydrofolate reductase isoform X3) c4(338017) c5( ) c6( ) c7(T, ahf, ea, bO, cgC, fd, ci, aY, bpI, Gt, aE, dV, JT, hu, hH, az, nY, YG, vs, aUG, cgz, fT, Jr, nB, lp, wE, Ji, cj, cX, k, eg, bzN, aaJ, ht, cQ, aj, aBP, sW, Gw, aaL, mR, sp, vu, ge, fe, aMJ, zZ, hq, vU, bbz, dB, se, vL, aoB, E, bFV, jv, aA, cz, bX, Eh, q, oT, akU, cy, rz, bqe, eu, bA, arI, bf, iy, eS, cl, pt, pb, cT, rR, iM, tW, ua, ac, bpv, mG, Nb, hr, tz, bt, gV, iW, Jy, jh, dG, uu, fu, ad, wD, hX, vJ, abq, oW, qV, lQ, BO, cH, cW, ff, aLI, dq, bpA, Lk, iV, bz, iq, DT, qe, ag, dX, Pk, lC, ne, bM, qo, aSz, if, e, bB, cL, ame, ur, ss, ye, ip, x, Na, vV, vh, fS, Gr, azm, dH, Jl, fc, hl, cgv, kt, Au, NR, Eg, eE, eF, AV, vy, wx, dD, gU, n, oY, nA, aSv, awq, md, Mo, jz, ec, amJ, Bj, acA, gd, eV, hn, DU, aBR, hi, cgB, MX, A, PX, AQ, atf, cgF, ajo, vo, bpx, aap, bP, cA, PI, agl, hL, at, ut, fL, ER, sa, B, vQ, aCw, wl, ard, ex, fF, vv, v, Qo, V, Mp, aqt, sl, aDr, ae, afs, t, sk, eL, ck, UP, gX, yv, vi, HQ, dM, OR, acU, eq, adg, hd, py, qh, LA, hD, sj, d, je, hG, gW, Z, jb, Hv, dr, bLL, bF, gk, yE, gZ, Tw, y, cgD, pC, ng, aC, UH, fR, al, cgG, lj, gf, qj, M, K, wt, dF, eL, mH, aAX, bpR, caD, qS, mQ, wf, g, ve, uC, aaS, ou, aBO, U, uo, vn, m, P, FW, xu, sQ, sw, aw, NT, fN, BH, pM, abQ, KL, ft, vC, wb, Vb, ny, bsK, cgH, sq, Ff, bIP, ack, wd, aUP, cM, Zj, ar, kT, W, afc, aiS, Qz, Pr, it, ach, fM, wj, AL, fG, oe, jx, aSA, wo, j, aqf, uW, eA, J, o, er, lg, hb, nX, yg, Nc, bq, aRR, kl, Y, r, cfm, en, wA, iK, bN, cgy, sn, c, cS, rK, dy, fD, lw, gT, gx, sY, kh, Gl, id, aZ, Gd, bS, ah, hF, gb, fU, tt, cx, adx, vl, WW, uX, BN, lR, Vx, cr, hp, Pd, iR, qa, cgE, cgA, X, cP, ei, hE, st, pf, fV, eb, aiC, Jx, mF, aR, aEQ, XW, dY, iI, ZX, qM, dQ, pk, eN, Bp, hm, zQ, LC, aW, da, lm, aVc, eM, iT, sU, sv, bqh, pw, cgw, bpH, vl, bzb, ez, mN, fr, ct, qN, ai, qg, ql, h, dL, aDC, ux, he, Df, Gc, wC, qY, aq, Ll, dw, bJ, vD, bpC, gY, hk, bm, gw, uz, eQ, bD, arg, bL, eR, dk, yk, rf, sM, cR, hy, C, bpW, ga, uT, aoA, aT, HG, vH, dz, bK, an, qk, af, bkl, qw, Uf, s, ND, iU, f, sS, wg, pZ, fP, aJY, mf, ij, cgx, uq, Zl, qb, ha, vr, tK, mC, kL, av, nH, F, ib, sO, NS, ts, dm, vw, fb, iL, eZ, yy, gl, aFr, aF, S, ek, es, apM, aAS, bE, pE, aS, GF, gq, us); #c1(MTHFR) c2(XP_011539798) c3(methylenetetrahydrofolate reductase isoform X4) c4( ) c5(338018, 338019, 338020, 338021, 338022, 338023, 338024, 338025) c6( ) c7(T, ahf, ea, bO, cgC, fd, ci, aY, bpl, Gt, aE, dV, JT, hu, hH, az, nY, YG, vs, aUG, cgz, fT, Jr, nB, lp, wE, Ji, cj, cX, k, eg, bzN, aaJ, ht, cQ, aj, aBP, sW, Gw, aaL, mR, sp, vu, ge, fe, aMJ, zZ, hq, vU, bbz, dB, se, vL, aoB, E, bFV, jv, aA, cz, bX, Eh, q, oT, akU, cy, rz, bqe, eu, bA, arI, bf, iy, eS, cl, pt, pb, cT, rR, iM, tW, ua, ac, bpv, mG, Nb, hr, tz, bt, gV, iW, Jy, jh, dG, uu, fu, ad, wD, hX, vJ, abq, oW, qV, lQ, BO, cH, cW, ff, aLI, dq, bpA, Lk, iV, bz, iq, DT, qe, ag, dX, Pk, lC, ne, bM, qo, aSz, if, e, bB, cL, ame, ur, ss, ye, ip, x, Na, vV, vh, fS, Gr, azm, dH, Jl, fc, hl, cgv, kt, Au, NR, Eg, eE, eF, AV, vy, wx, dD, gU, n, oY, nA, aSv, awq, md, Mo, jz, ec, amJ, Bj, acA, gd, eV, hn, DU, aBR, hi, cgB, MX, A, PX, AQ, atf, cgF, ajo, vo, bpx, aap, bP, cA, Pl, agl, hL, at, ut, fL, ER, sa, B, vQ, aCw, wL, ard, eX, fF, vv, v, Qo, V, Mp, aqt, sl, aDr, ae, afs, t, sK, el, ck, UP, gX, yv, vi, HQ, dM, oR, acU, eq, adg, hd, py, qh, LA, hD, sj, d, je, hG, gW, Z, jb, Hv, dr, bLL, bF, gk, yE, gZ, Tw, y, cgD, pC, ng, aC, UH, fR, al, cgG, lj, gf, qj, M, K, wt, dF, eL, mH, aAX, bpR, caD, qS, mQ, wf, g, ve, uC, aaS, ou, aBO, U, uo, vn, m, P, FW, xu, sQ, sw, aw, NT, fN, BH, pM, abQ, KL, ft, vC, wb, Vb, ny, bsK, cgH, sq, Ff, bIP, ack, wd, aUP, cM, Zj, ar, kT, W, afc, aiS, Qz, Pr, it, ach, fM, wj, AL, fG, oe, jx, aSA, wo, j, aqf, uW, eA, J, o, er, lg, hb, nX, yg, Nc, bq, aRR, kl, Y, r, cfm, en, wA, iK, bN, cgy, sn, c, cS, rK, dy, fD, lw, gT, gx, sY, kh, Gl, id, aZ, Gd, bS, ah, hF, gb, fU, tt, cx, adx, vl, WW, uX, BN, lR, Vx, cr, hp, Pd, iR, qa, cgE, cgA, X, cP, ei, hE, st, pf, fV, eb, aiC, Jx, mF, aR, aEQ, XW, dY, iL, ZX, qM, dQ, pk, eN, Bp, hm, zQ, LC, aW, da, lm, aVc, eM, iT, sU, sv, bqh, pw, cgw, bpH, vl, bzb, ez, mN, fr, ct, qN, ai, qg, ql, h, dL, aDC, ux, he, Df, Gc, wC, qY, aq, Ll, dw, bJ, vD, bpC, gY, hk, bm, gw, uz, eQ, bD, arg, bL, eR, dk, yk, rf, sM, cR, hy, C, bpW, ga, uT, aoA, aT, HG, vH, dz, bk, an, qk, af, bkl, qw, Uf, s, ND, iU, f, sS, wg, pZ, fP, aJY, mf, ij, cgx, uq, Zl, qb, ha, vr, tK, mC, kL, av, nH, F, ib, sO, NS, ts, dm, vw, fb, iL, eZ, yy, gl, aFr, aF, S, ek, es, apM, aAS, bE, pE, aS, GF, gq, us); #c1(MTHFS) c2(NP_006432) c3(5-formyltetrahydrofolate cyclo-ligase isoform a) c4(165196) c5(165197, 165198, 165199, 165200, 165201, 165202) c6( ) c7(Na, fM, eR, lp, iW, e, r, Nc, t); #c1(MTIF2) c2(NP_001005369) c3(translation initiation factor IF-2, mitochondrial precursor) c4(21526, 21527, 21528, 21529, 21530, 21531) c5(21532, 21533, 21534, 21535, 21536, 21537, 21538, 21539, 21540, 21541, 21542, 21543, 21544, 21545, 21546, 21547, 21548, 21549, 21550, 21551, 21552, 21553, 21554, 21555, 21556, 21557, 21558, 21559, 21560, 21561, 21562, 21563, 21564, 21565, 21566, 21567, 21568, 21569) 21570, 21571, 21572, 21573, 21574, 21575, 21576, 21577, 21578) c6( ) c7(bL); #c1(MTIF3) c2(XP_011533265) c3(translation initiation factor IF-3, mitochondrial isoform X1) c4(329442) c5(329443, 329444, 329445, 329446, 329447, 329448, 329449, 329450, 329451, 329452, 329453, 329454, 329455, 329456, 329457, 329458, 329459, 329460) c6( ) c7(bL, cm, fq, v, an); #c1(MTL5) c2(NP_001034745) c3(tesmin isoform b) c4(32192) c5(32193, 32194, 32195, 32196, 32197, 32198, 32199, 32200, 32201, 32202, 32203, 32204) c6( ) c7(aw); #c1(MTL5) c2(NP_004914) c3(tesmin isoform a) c4(150722) c5(150723, 150724, 150725, 150726, 150727, 150728) c6( ) c7(aw); #c1(MTL5) c2(XP_011543705) c3(tesmin isoform X1) c4(342784) c5( ) c6( ) c7(aw); #c1(MTM1) c2(XP_011529475) c3(myotubularin isoform X1) c4(324891, 324892, 324893, 324894) c5(324895, 324896, 324897, 324898, 324899, 324900, 324901, 324902, 324903, 324904, 324905, 324906, 324907, 324908, 324909, 324910, 324911, 324912, 324913, 324914, 324915, 324916, 324917, 324918, 324919, 324920, 324921, 324922, 324923) c6( ) c7(T, cgk, cgJ, KW, aA, fu, cgl, bX, UX, no, oj, Ak, bI, K, bU, li, xz, Ad, dr, dR, mx, aWn, CD, ei); #c1(MTMR1) c2(NP_003819) c3(myotubularin-related protein 1) c4(141126, 141127, 141128) c5(141129, 141130, 141131, 141132, 141133, 141134, 141135, 141136, 141137, 141138, 141139, 141140, 141141, 141142, 141143, 141144, 141145, 141146, 141147) c6( ) c7(xz, dR); #c1(MTMR1) c2(XP_011529510) c3(myotubularin-related protein 1 isoform X7) c4(325042) c5( ) c6( ) c7(xz, dR); #c1(MTMR1) c2(XP_011529512) c3(myotubularin-related protein 1 isoform X9) c4(325043) c5(325044, 325045, 325046) c6( ) c7(xz, dR); #c1(MTMR11) c2(XP_011507401) c3(myotubularin-related protein 11 isoform X4) c4(297267) c5(297268, 297269, 297270, 297271, 297272) c6( ) c7(ha, T, q, bv, r); #c1(MTMR11) c2(XP_011507402) c3(myotubularin-related protein 11 isoform X5) c4(297273, 297274) c5(297275, 297276, 297277, 297278, 297279, 297280, 297281, 297282, 297283, 297284, 297285) c6( ) c7(ha, T, q, bv, r); #c1(MTMR12) c2(NP_001035536) c3(myotubularin-related protein (2 isoform 1) c4(32887) c5(32888, 32889, 32890, 32891, 32892, 32893) c6( ) c7(xz, UX); #c1(MTMR12) c2(NP_001281272) c3(myotubularin-related protein 12 isoform 1) c4(111662) c5(111663, 111664, 111665, 111666, 111667, 111668) c6( ) c7(xz, UX); #c1(MTMR12) c2(NP_001281273) c3(myotubularin-related protein 12 isoform 3) c4(111669) c5(111670, 111671, 111672, 111673, 111674, 111675) c6( ) c7(xz, UX); #c1(MTMR14) c2(NP_001070993) c3(myotubularin-related protein 14 isoform 2) c4(35241) c5(35242, 35243, 35244, 35245, 35246, 35247, 35248) c6( ) c7(xz, bhe, Km, oj, UX); #c1(MTMR14) c2(NP_001070994) c3(myotubularin-related protein 14 isoform 1) c4(35249, 35250) c5(35251, 35252, 35253, 35254, 35255, 35256) c6( ) c7(xz, bhe, Km, oj, UX); #c1(MTMR14) c2(NP_071930) c3(myotubularin-related protein 14 isoform 3) c4(212368) c5(212369, 212370, 212371, 212372, 212373, 212374, 212375, 212376, 212377, 212378, 212379) c6( ) c7(xz, bhe, Km, oj, UX); #c1(MTMR2) c2(NP_057240) c3(myotubularin-related protein 2 isoform 1) c4(189670, 189671, 189672) c5(189673, 189674, 189675, 189676, 189677, 189678, 189679, 189680, 189681, 189682, 189683, 189684, 189685, 189686, 189687) c6( ) c7(ql, Pt, agU, xz, U, bbH, bk, fu, cgl, bU, dw, bX, UX); #c1(MTMR2) c2(NP_958438) c3(myotubularin-related protein 2 isoform 2) c4( ) c5(262099, 262100, 262101, 262102, 262103, 262104, 262105, 262106, 262107, 262108, 262109) c6( ) c7(ql, Pt, agU, xz, U, bbH, bk, fu, cgl, bU, dw, bX, UX); #c1(MTMR2) c2(XP_011541361) c3(myotubularin-related protein 2 isoform X2) c4(339950) [5( ) c6( ) c7(ql, Pt, agU, xz, U, bbH, bk, fu, cgl, bU, dw, bX, UX); #c1(MTMR3) c2(NP_066576) c3(myotubularin-related protein 3 isoform c) c4(207450) c5(207451, 207452, 207453, 207454, 207455, 207456) c6( ) c7(av, T, aA, aF, ey, K, bz); #c1(MTMR3) c2(NP_694690) c3(myotubularin-related protein 3 isoform a) c4(245353) c5(245354, 245355, 245356, 245357, 245358, 245359, 245360, 245361, 245362, 245363) c6( ) c7(av, T, aA, aF, ey, K, bz); #c1(MTMR3) c2(NP_694691) c3(myotubularin-related protein 3 isoform b) c4(245364, 245365) c5(245366, 245367, 245368, 245369, 245370, 245371, 245372, 245373, 245374, 245375, 245376, 245377) c6( ) c7(av, T, aA, aF, ey, K, bz); #c1(MTMR3) c2(XP_005261866) c3(myotubularin-related protein 3 isoform X6) c4( ) c5(277646) c6( ) c7(av, T, aA, aF, ey, K, bz); #c1(MTMR4) c2(XP_006722231) c3(myotubularin-related protein 4 isoform X1) c4( ) c5(294655, 294656, 294657, 294658, 294659, 294660) c6( ) c7(ki, bl); #c1(MTMR4) c2(XP_011523762) c3(myotubularin-related protein 4 isoform X3) c4(318258) c5( ) c6( ) c7(ki, bl); #c1(MTMR6) c2(NP_004676) c3(myotubularin-related protein 6) c4(148745, 148746, 148747) c5(148748, 148749, 148750, 148751, 148752, 148753, 148754, 148755, 148756, 148757, 148758, 148759, 148760, 148761, 148762, 148763, 148764, 148765, 148766, 148767, 148768, 148769, 148770) c6( ) c7(T); #c1(MTMR6) c2(XP_011533608) c3(myotubularin-related protein 6 isoform X1) c4(329719) c5( ) c6( ) c7(T); #c1(MTMR6) c2(XP_011533609) c3(myotubularin-related protein 6 isoform X2) c4( ) c5(329720) c6( ) c7(T); #c1(MTMR7) c2(NP_004677) c3(myotubularin-related protein 7) c4(148771, 148772) c5(148773, 148774, 148775, 148776, 148777, 148778, 148779, 148780, 148781, 148782, 148783, 148784) c6( ) c7(fq, dS); #c1(MTMR7) c2(XP_011542995) c3(myotubularin-related protein 7 isoform X3) c4(341686) c5(341687, 341688, 341689, 341690, 341691, 341692) c6( ) c7(fq, dS); #c1(MTMR8) c2(NP_060147) c3(myotubularin-related protein 8) c4(194459, 194460, 194461, 194462) c5(194463, 194464, 194465, 194466, 194467, 194468, 194469, 194470, 194471, 194472, 194473, 194474, 194475, 194476, 194477, 194478, 194479, 194480, 194481, 194482, 194483, 194484, 194485, 194486) c6( ) c7(W); #c1(MTMR9) c2(NP_056273) c3(myotubularin-related protein 9) c4(186849) c5(186850, 186851, 186852, 186853, 186854, 186855) c6( ) c7(v, cz); #c1(MTNR1A) c2(NP_005949) c3(melatonin receptor type (A) c4(160618) c5(160619, 160620, 160621, 160622, 160623, 160624) c6( ) c7(bL, FG, iW, PJ, HF, aT, rz, af, B, VX, lR, dH, aW, ff, HL, en, bQ, eF, bN, dS, eM, Km, y, X); #c1(MTNR1B) c2(NP_005950) c3(melatonin receptor type (B) c4(160625) c5(160626, 160627, 160628, 160629, 160630, 160631) c6( ) c7(bL, dD, FG, aZ, Nt, GP, aj, bS, dG, ct, v, eq, J, HF, no, aT, h, dL, wb, ji, rz, eL, kl, VX, aMG, aW, Y, Mz, HL, bQ, Z, bN, iP, mG, mg, mf, ei, y); #c1(MTO1) c2(NP_001116698) c3(protein MTO1 homolog, mitochondrial isoform c) c4(43941) c5(43942, 43943, 43944, 43945, 43946, 43947) c6( ) c7(bL, T, cc, SE, aW, cgL, mG, aT, bZ, vc); #c1(MT01) c2(NP_036255) c3(protein MTO1 homolog, mitochondrial isoform a) c4(172662) c5(172663, 172664, 172665, 172666, 172667, 172668) c6( ) c7(bL, T, cc, SE, aW, cgL, mG, aT, bZ, vc); #c1(MT01) c2(NP_598400) c3(protein MTO1 homolog, mitochondrial isoform b) c4(235503, 235504) c5(235505, 235506, 235507, 235508, 235509, 235510, 235511, 235512, 235513, 235514, 235515, 235516) c6( ) c7(bL, T, cc, SE, aW, cgL, mG, aT, bZ, vc); #c1(MT01) c2(XP_006715508) c3 (protein MTO1 homolog, mitochondrial isoform X1) c4(289603) c5( ) c6( ) c7(bL, T, cc, SE, aW, cgL, mG, aT, bZ, vc); #c1(MTO1) c2(XP_011534027) c3(protein MTO1 homolog, mitochondrial isoform X3) c4( ) c5(330201, 330202, 330203, 330204, 330205, 330206) c6( ) c7(bL, T, cc, SE, aW, cgL, mG, aT, bZ, vc); #c1(MTPAP) c2(NP_060579) c3(poly(A) RNA polymerase, mitochondrial precursor) c4(196638, 196639, 196640, 196641) c5(196642, 196643, 196644, 196645, 196646, 196647, 196648, 196649, 196650, 196651, 196652, 196653, 196654, 196655, 196656, 196657, 196658, 196659, 196660, 196661, 196662, 196663, 196664, 196665) c6( ) c7(ck, A, d, dA, af, Y, iW, r, wq, dT, awK, aj, zG, uF, v, zl, cgM, cd, ao); #c1(MTPN) c2(NP_665807) c3(myotrophin) c4(241709) c5(241710, 241711, 241712, 241713, 241714, 241715) c6( ) c7(cd, cz); #c1(MTR) c2(NP_000245) c3(methionine synthase isoform 1) c4(5590) c5(5591, 5592, 5593, 5594, 5595, 5596) c6( ) c7(ng, T, aC, pf, fV, if, e, al, fd, bB, ur, ci, mF, aR, ip, x, Na, aE, iX, fS, M, dV, K, hu, dQ, dF, eL, eN, aW, caD, cgv, da, g, eF, uC, cgz, eM, lp, dc, wE, sv, k, P, dt, gU, dD, aw, ht, oY, mN, ct, qN, abQ, md, bJf, Mo, ft, amJ, Bj, dL, ey, MX, fe, bJ, W, bP, gY, dB, bm, at, my, bD, Qz, it, B, cn, bL, fM, eR, mr, aA, sM, cz, hy, v, j, bX, V, q, eA, XX, MP, yg, aT, HG, vH, Nc, gz, bK, bq, an, avl, ae, bkl, af, eu, Uf, kl, s, ND, r, en, f, iK, bN, ck, ahN, rR, ac, bpv, mG, gX, dy, ij, bt, gV, id, iW, ha, jh, acU, bS, uu, eq, ad, av, abq, iL, d, BN, aAA, hG, ff, eZ, dq, bpA, aF, aDd, iV, Z, jb, bz, qa, bE, aS, Tw, GF, y, ne, us); #c1(MTR) c2(NP_001278868) c3 (methionine synthase isoform 2) c4(110743) c5(110744, 110745, 110746, 110747, 110748, 110749) c6( ) c7(ng, T, aC, pf, fV, if, e, al, fd, bB, ur, ci, mF, aR, ip, x, Na, aE, iX, fS, M, dV, K, hu, dQ, dF, eL, eN, aW, caD, cgv, da, g, eF, uC, cgz, eM, lp, dc, wE, sv, k, P, dt, gU, dD, aw, ht, oY, mN, ct, qN, abQ, md, bJf, Mo, ft, amJ, Bj, dL, ey, MX, fe, bJ, W, bP, gY, dB, bm, at, my, bD, Qz, it, B, cn, bL, fM, eR, mr, aA, sM, cz, hy, v, j, bX, V, q, eA, XX, MP, yg, aT, HG, vH, Nc, gz, bK, bq, an, avl, ae, bkl, af, eu, Uf, kl, s, ND, r, en, f, iK, bN, ck, ahN, rR, ac, bpv, mG, gX, dy, ij, bt, gV, id, iW, ha, jh, acU, bS, uu, eq, ad, av, abq, iL, d, BN, aAA, hG, ff, eZ, dq, bpA, aF, aDd, iV, Z, jb, bz, qa, bE, aS, Tw, GF, y, ne, us); #c1(MTR) c2(XP_005273198) c3 (methionine synthase isoform X1) c4(284333, 284334) c5( ) c6( ) c7(ng, T, aC, pf, fV, if, e, al, fd, bB, ur, ci, mF, aR, ip, x, Na, aE, iX, fS, M, dV, K, hu, dQ, dF, eL, eN, aW, caD, cgv, da, g, eF, uC, cgz, eM, lp, dc, wE, sv, k, P, dt, gU, dD, aw, ht, oY, mN, ct, qN, abQ, md, bJf, Mo, ft, amJ, Bj, dL, ey, MX, fe, bJ, W, bP, gY, dB, bm, at, my, bD, Qz, it, B, cn, bL, fM, eR, mr, aA, sM, cz, hy, v, j, bX, V, q, eA, XX, MP, yg, aT, HG, vH, Nc, gz, bK, bq, an, avl, ae, bkl, af, eu, Uf, kl, s, ND, r, en, f, iK, bN, ck, ahN, rR, ac, bpv, mG, gX, dy, ij, bt, gV, id, iW, ha, jh, acU, bS, uu, eq, ad, av, abq, iL, d, BN, aAA, hG, ff, eZ, dq, bpA, aF, aDd, iV, Z, jb, bz, qa, bE, aS, Tw, GF, y, ne, us); #c1(MTR) c2(XP_011542496) c3(methionine synthase isoform X5) c4(341021) c5(341022, 341023, 341024, 341025, 341026, 341027, 341028, 341029, 341030, 341031, 341032, 341033, 341034, 341035, 341036, 341037, 341038, 341039) c6( ) c7(ng, T, aC, pf, fV, if, e, al, fd, bB, ur, ci, mF, aR, ip, x, Na, aE, iX, fS, M, dV, K, hu, dQ, dF, eL, eN, aW, caD, cgv, da, g, eF, uC, cgz, eM, lp, dc, wE, sv, k, P, dt, gU, dD, aw, ht, oY, mN, ct, qN, abQ, md, bJf, Mo, ft, amJ, Bj, dL, ey, MX, fe, bJ, W, bP, gY, dB, bm, at, my, bD, Qz, it, B, cn, bL, fM, eR, mr, aA, sM, cz, hy, v, j, bX, V, q, eA, XX, MP, yg, aT, HG, vH, Nc, gz, bK, bq, an, avl, ae, bkl, af, eu, Uf, kl, s, ND, r, en, f, iK, bN, ck, ahN, rR, ac, bpv, mG, gX, dy, ij, bt, gV, id, iW, ha, jh, acU, bS, uu, eq, ad, av, abq, iL, d, BN, aAA, hG, ff, eZ, dq, bpA, aF, aDd, iV, Z, jb, bz, qa, bE, aS, Tw, GF, y, ne, us); #c1(MTRF1) c2(XP_006719963) c3(peptide chain release factor 1, mitochondrial isoform X1) c4( ) c5(293108, 293109, 293110, 293111, 293112, 293113, 293114, 293115) c6( ) c7(aY, eR); #c1(MTRF1) c2(XP_011533617) c3(peptide chain release factor 1, mitochondrial isoform X2) c4(329723) c5(329724) c6( ) c7(aY, eR); #c1(MTRF1) c2(XP_011533618) c3(peptide chain release factor 1, mitochondrial isoform X3) c4(329725, 329726) c5(329727, 329728, 329729, 329730, 329731, 329732) c6( ) c7(aY, eR); #c1(MTRF1) c2(XP_011533619) c3(peptide chain release factor 1, mitochondrial isoform X4) c4( ) c5(329733) c6( ) c7(aY, eR); #c1(MTRF1) c2(XP_011533629) c3 (peptide chain release factor 1, mitochondrial isoform X6) c4( ) c5(329734, 329735, 329736, 329737, 329738, 329739) c6( ) c7(aY, eR); #c1(MTRNR2L7) c2(NP_001177418) c3(humanin-like 7) c4(72792) c5(72793, 72794, 72795, 72796, 72797, 72798) c6( ) c7(v); #c1(MTRR) c2(NP_002445) c3(methionine synthase reductase isoform 1) c4( ) c5(128497, 128498, 128499, 128500, 128501) c6( ) c7(ng, T, aC, fV, ea, e, if, fd, bB, ur, mF, aR, ip, x, Na, aE, fS, M, K, dF, eL, aW, caD, da, g, uC, cgz, eM, cgN, lp, wE, cj, sv, k, P, dD, gU, aw, n, ct, qN, cd, md, Bl, b, ft, i, Bj, hn, dL, ey, MX, A, fe, aq, Ll, bbz, W, gY, dB, bm, at, bD, Pr, B, it, aA, sM, cz, hy, v, j, V, q, eA, MP, aT, HG, vH, Nc, bK, an, ae, af, eu, bf, s, ND, Y, r, en, f, iK, bN, ck, rR, ac, UP, mG, dy, bt, iW, ha, acU, bS, eq, uu, ad, hX, av, fb, iL, ff, eZ, dq, Lk, aF, iV, Z, jb, bz, dr, bE, pE, aS, Tw, GF, y, BA); #c1(MTRR) c2(NP_076915) c3(methionine synthase reductase isoform 2) c4(214204, 214205) c5(214206, 214207, 214208, 214209, 214210, 214211, 214212) c6( ) c7(ng, T, aC, fV, ea, e, if, fd, bB, ur, mF, aR, ip, x, Na, aE, fS, M, K, dF, eL, aW, caD, da, g, uC, cgz, eM, cgN, lp, wE, cj, sv, k, P, dD, gU, aw, n, ct, qN, cd, md, Bl, b, ft, i, Bj, hn, dL, ey, MX, A, fe, aq, Ll, bbz, W, gY, dB, bm, at, bD, Pr, B, it, aA, sM, cz, hy, v, j, V, q, eA, MP, aT, HG, vH, Nc, bK, an, ae, af, eu, bf, s, ND, Y, r, en, f, iK, bN, ck, rR, ac, UP, mG, dy, bt, iW, ha, acu, bS, eq, uu, ad, hX, av, fb, iL, ff, eZ, dq, Lk, aF, iV, Z, jb, bz, dr, bE, pE, aS, Tw, GF, y, BA); #c1(MTRR) c2(XP_006714537) c3(methionine synthase reductase isoform X3) c4( ) c5(287900) c6( ) c7(ng, T, aC, fV, ea, e, if, fd, bB, ur, mF, aR, ip, x, Na, aE, fS, M, K, dF, eL, aW, caD, da, g, uC, cgz, eM, cgN, lp, wE, cj, sv, k, P, dD, gU, aw, n, ct, qN, cd, md, Bl, b, ft, i, Bj, hn, dL, ey, MX, A, fe, aq, Ll, bbz, W, gY, dB, bm, at, bD, Pr, B, it, aA, sM, cz, hy, v, j, V, q, eA, MP, aT, HG, vH, Nc, bK, an, ae, af, eu, bf, s, ND, Y, r, en, f, iK, bN, ck, rR, ac, UP, mG, dy, bt, iW, ha, acu, bS, eq, uu, ad, hX, av, fb, iL, ff, eZ, dq, Lk, aF, iV, Z, jb, bz, dr, bE, pE, aS, Tw, GF, y, BA); #c1(MTRR) c2(XP_011512347) c3(methionine synthase reductase isoform X4) c4(303023, 303024, 303025, 303026) c5(303027, 303028, 303029, 303030, 303031, 303032, 303033, 303034, 303035, 303036, 303037, 303038, 303039, 303040, 303041, 303042, 303043, 303044, 303045, 303046, 303047, 303048, 303049) c6( ) c7(ng, T, aC, fV, ea, e, if, fd, bB, ur, mF, aR, ip, x, Na, aE, fS, M, K, dF, eL, aW, caD, da, g, uC, cgz, eM, cgN, lp, wE, cj, sv, k, P, dD, gU, aw, n, ct, qN, cd, md, Bl, b, ft, i, Bj, hn, dL, ey, MX, A, fe, aq, Ll, bbz, W, gY, dB, bm, at, bD, Pr, B, it, aA, sM, cz, hy, v, j, V, q, eA, MP, aT, HG, vH, Nc, bK, an, ae, af, eu, bf, s, ND, Y, r, en, f, iK, bN, ck, rR, ac, UP, mG, dy, bt, iW, ha, acU, bS, eq, uu, ad, hX, av, fb, iL, ff, eZ, dq, Lk, aF, iV, Z, jb, bz, dr, bE, pE, aS, Tw, GF, y, BA); #c1(MTSS1) c2(NP_001269903) c3(metastasis suppressor protein 1 isoform 3) c4(103117) c5(103118, 103119, 103120, 103121, 103122, 103123) c6( ) c7(jf, T, B, aW, Y, eL, iW, ff, r, aT, aA, dL, af, ci, en, ae, q, ad); #c1(MTSS1) c2(NP_055566) c3(metastasis suppressor protein 1 isoform 2) c4(181961, 181962) c5(181963, 181964, 181965, 181966, 181967, 181968, 181969, 181970, 181971, 181972, 181973, 181974) c6( ) c7(jf, T, B, aW, Y, eL, iW, ff, r, aT, aA, dL, af, ci, en, ae, q, ad); #c1(MTSS1) c2(XP_006716769) c3(metastasis suppressor protein 1 isoform X12) c4(290447) c5(290448) c6( ) c7(jf, T, B, aW, Y, eL, iW, ff, r, aT, aA, dL, af, ci, en, ae, q, ad); #c1(MTSS1) c2(XP_011515710) c3(metastasis suppressor protein 1 isoform X16) c4( ) c5(308547, 308548, 308549, 308550, 308551) c6( ) c7(jf, T, B, aW, Y, eL, iW, ff, r, aT, aA, dL, af, ci, en, ae, q, ad); #c1(MTTP) c2(NP_000244) c3(microsomal triglyceride transfer protein large subunit isoform 1 precursor) c4(5423, 5424, 5425, 5426) c5(5427, 5428, 5429, 5430, 5431, 5432, 5433, 5434, 5435, 5436, 5437, 5438, 5439, 5440, 5441, 5442, 5443, 5444, 5445, 5446, 5447, 5448, 5449, 5450, 5451, 5452, 5453, 5454, 5455, 5456, 5457, 5458, 5459, 5460, 5461, 5462, 5463, 5464, 5465, 5466, 5467, 5468, 5469, 5470, 5471, 5472, 5473, 5474, 5475, 5476, 5477, 5478, 5479, 5480, 5481, 5482, 5483, 5484, 5485, 5486, 5487, 5488, 5489, 5490, 5491, 5492, 5493, 5494, 5495, 5496, 5497, 5498, 5499, 5500, 5501, 5502, 5503, 5504, 5505, 5506, 5507, 5508, 5509, 5510, 5511, 5512, 5513, 5514, 5515, 5516, 5517, 5518, 5519, 5520, 5521, 5522, 5523, 5524, 5525, 5526, 5527, 5528, 5529, 5530, 5531, 5532, 5533, 5534, 5535, 5536, 5537, 5538, 5539, 5540, 5541, 5542, 5543, 5544, 5545, 5546, 5547, 5548, 5549, 5550, 5551, 5552, 5553, 5554, 5555, 5556, 5557, 5558, 5559, 5560, 5561, 5562, 5563, 5564, 5565, 5566, 5567, 5568, 5569, 5570, 5571, 5572, 5573, 5574, 5575, 5576, 5577, 5578, 5579, 5580, 5581, 5582, 5583, 5584, 5585, 5586, 5587, 5588) c6(5589) c7(T, ea, e, al, fd, GK, x, oB, pp, K, hu, dQ, pk, aW, fc, hl, bw, gn, k, dD, gp, aw, ht, n, aj, oY, ct, pn, pe, ec, h, jy, ox, ZN, on, cgO, aq, bJ, oU, W, bP, Fi, ZG, B, bL, ZV, dA, oe, jx, cz, ol, v, j, C, nS, q, J, er, aT, dz, iU, oV, r, lo, hf, fi, ck, iM, mf, gT, bt, hl, ow, jh, oR, bS, eq, jE, fu, hF, hX, F, fX, ZU, fb, iL, d, je, et, dq, Z, dr, gZ, cP, ei); #c1(MTURN) c2(NP_690006) c3(maturin) c4(244718, 244719) c5(244720, 244721, 244722, 244723, 244724, 244725, 244726, 244727, 244728, 244729, 244730, 244731, 244732, 244733, 244734, 244735, 244736, 244737, 244738, 244739, 244740) c6( ) c7(aw); #c1(MTUS1) c2(NP_001001931) c3(microtubule-associated tumor suppressor 1 isoform 4) c4(19170) c5(19171, 19172, 19173, 19174, 19175, 19176, 19177) c6( ) c7(T, aW, eL, aw, bzi, aT, dL, ci, m, g, ae, B, ad); #c1(MTUS1) c2(NP_001159865) c3(microtubule-associated tumor suppressor 1 isoform 6) c4( ) c5(64863, 64864, 64865, 64866, 64867, 64868, 64869, 64870, 64871, 64872, 64873) c6( ) c7(T, aW, eL, aw, bzi, aT, dL, ci, m, g, ae, B, ad); #c1(MTUS1) c2(NP_065800) c3(microtubule-associated tumor suppressor 1 isoform 5) c4(205554) c5(205555, 205556, 205557, 205558, 205559, 205560) c6( ) c7(T, aW, eL, aw, bzi, aT, dL, ci, m, g, ae, B, ad); #c1(MTUS1) c2(XP_005273636) c3(microtubule-associated tumor suppressor 1 isoform X2) c4(284722, 284723, 284724, 284725) c5(284726, 284727, 284728, 284729, 284730, 284731) c6( ) c7(T, aW, eL, aw, bzi, aT, dL, ci, m, g, ae, B, ad); #c1(MTUS1) c2(XP_005273639) c3(microtubule-associated tumor suppressor 1 isoform X1) c4(284732) c5(284733, 284734, 284735, 284736, 284737, 284738, 284739, 284740, 284741, 284742) c6( ) c7(T, aW, eL, aw, bzi, aT, dL, ci, m, g, ae, B, ad); #c1(MTUS1) c2(XP_005273640) c3(microtubule-associated tumor suppressor 1 isoform X6) c4( ) c5(284743, 284744, 284745, 284746, 284747, 284748) c6( ) c7(T, aW, eL, aw, bzi, aT, dL, ci, m, g, ae, B, ad); #c1(MTUS2) c2(NP_001028774) c3(microtubule-associated tumor suppressor candidate 2 isoform a) c4(30649, 30650) c5(30651, 30652, 30653, 30654, 30655, 30656) c6( ) c7(bM, oe, cd); #c1(MTUS2) c2(NP_056048) c3(microtubule-associated tumor suppressor candidate 2 isoform b) c4(185282) c5(185283, 185284, 185285, 185286, 185287, 185288, 185289, 185290, 185291, 185292, 185293, 185294, 185295, 185296, 185297, 185298, 185299, 185300) c6( ) c7(bM, oe, cd); #c1(MTUS2) c2(XP_011533323) c3(microtubule-associated tumor suppressor candidate 2 isoform X3) c4(329464) c5( ) c6( ) c7(bM, oe, cd); #c1(MTX1) c2(NP_002446) c3(metaxin-1 isoform 1) c4(128502, 128503) c5(128504, 128505, 128506, 128507, 128508, 128509, 128510, 128511, 128512, 128513, 128514, 128515) c6( ) c7(ck, vU, x, ac, zr, bE, aW, Y, wE, cr, aT, sM, bB, jh, k, ij, y, aen); #c1(MTX1) c2(NP_942584) c3(metaxin-1 isoform 2) c4(260558) c5(260559, 260560, 260561, 260562, 260563, 260564) c6( ) c7(ck, vU, x, ac, zr, bE, aW, Y, wE, cr, aT, sM, bB, jh, k, ij, y, aen); #c1(MTX2) c2(NP_006545) c3(metaxin-2) c4(166128, 166129) c5(166130, 166131, 166132, 166133, 166134, 166135, 166136, 166137, 166138, 166139, 166140, 166141) c6( ) c7(bk); #c1(MUC1) c2(NP_001018016) c3(mucin-1 isoform 2 precursor) c4(26845, 26846) c5(26847, 26848, 26849, 26850, 26851, 26852, 26853, 26854, 26855, 26856, 26857, 26858) c6( ) c7(oO, bM, T, aC, fR, pf, if, bKw, bB, fd, ci, aY, fw, wa, aE, u, rJ, aEJ, pX, bW, Ds, K, hu, eL, cp, YL, ZH, iE, Jl, aW, bKt, aoo, az, fc, kt, g, as, cm, Qx, fT, eM, nB, iA, m, uL, in, iu, cE, sw, iB, ht, fN, n, aj, fr, ct, qN, zo, ai, eW, Vs, Bl, ft, jz, aoc, ju, cU, bKs, lG, dL, h, cgl, bzs, mR, CN, aoX, MD, fe, kp, oU, at, it, B, Dw, bev, fM, dA, eR, fG, ard, DV, aA, fF, zN, v, ze, uW, AO, q, ccP, aKc, eJ, RU, lg, hb, afQ, aT, ae, jf, kl, s, NE, nO, oV, cgR, Y, ev, iU, r, aZg, en, avb, cl, LV, t, Px, iK, ck, nM, iM, gX, fP, dy, EY, cgP, bt, KY, rA, ao, hS, nZ, Mb, Pt, aD, ha, rl, jh, vm, bS, oH, Gn, iJ, vN, ad, dP, nH, l, F, hd, ib, Db, ds, io, ji, uD, vl, uQ, je, BO, qt, ff, rM, So, aF, iV, Z, bz, LB, es, mO, bF, bE, y, X, BA); #c1(MUC1) c2(NP_001018017) c3(mucin-1 isoform 3 precursor) c4(26859) c5(26860, 26861, 26862, 26863, 26864, 26865) c6( ) c7(oO, bM, T, aC, fR, pf, if, bKw, bB, fd, ci, aY, fw, wa, aE, u, rJ, aEJ, pX, bW, Ds, K, hu, eL, cp, YL, ZH, iE, Jl, aW, bKt, aoo, az, fc, kt, g, as, cm, Qx, fT, eM, nB, iA, m, uL, in, iu, cE, sw, iB, ht, fN, n, aj, fr, ct, qN, zo, ai, eW, Vs, Bl, ft, jz, aoc, ju, cl, bKs, IG, dL, h, cgQ, bzs, mR, CN, aoX, MD, fe, kp, oU, at, it, B, Dw, bev, fM, dA, eR, fG, arQ, DV, aA, fF, zN, v, ze, uW, AO, q, ccP, aKc, eJ, RU, lg, hb, afQ, aT, ae, jf, kl, s, NE, nO, oV, cgR, Y, ev, iU, r, aZg, en, avb, cl, LV, t, Px, iK, ck, nM, iM, gX, fP, dy, EY, cgP, bt, KY, rA, ao, hS, nZ, Mb, Pt, aD, ha, rl, jh, vm, bS, oH, Gn, iJ, vN, ad, dP, nH, l, F, hd, ib, Db, ds, io, ji, uD, vl, uQ, je, BO, qt, ff, rM, So, aF, iV, Z, bz, LB, es, mO, bF, bE, y, X, BA); #c1(MUC1) c2(NP_001037855) c3(mucin-1 isoform 5 precursor) c4(34157) c5(34158, 34159, 34160, 34161, 34162, 34163) c6( ) c7(oO, bM, T, aC, fR, pf, if, bKw, bB, fd, ci, aY, fw, wa, aE, u, rJ, aEJ, pX, bW, Ds, K, hu, eL, cp, YL, ZH, iE, Jl, aW, bKt, aoo, az, fc, kt, g, as, cm, Qx, fT, eM, nB, iA, m, ul, in, iu, cE, sw, iB, ht, fN, n, aj, fr, ct, qN, zo, ai, eW, Vs, Bl, ft, jz, aoc, ju, cU, bKs, lG, dL, h, cgQ, bzs, mR, CN, aoX, MD, fe, kp, oU, at, it, B, Dw, bev, fM, dA, eR, fG, arQ, DV, aA, fF, zN, v, ze, uW, AO, q, ccP, aKc, eJ, RU, lg, hb, afQ, aT, ae, jf, kl, s, NE, nO, oV, cgR, Y, ev, iU, r, aZg, en, avb, cl, LV, t, Px, iK, ck, nM, iM, gX, fP, dy, EY, cgP, bt, KY, rA, ao, hS, nZ, Mb, Pt, aD, ha, rl, jh, vm, bS, oH, Gn, iJ, vN, ad, dP, nH, l, F, hd, ib, Db, ds, io, ji, uD, vl, uQ, je, BO, qt, ff, rM, So, aF, iV, Z, bz, LB, es, mO, bF, bE, y, X, BA); #c1(MUC1) c2(NP_001037857) c3(mucin-1 isoform 7 precursor) c4(34164) c5(34165, 34166, 34167, 34168, 34169, 34170) c6( ) c7(oO, bM, T, aC, fR, pf, if, bKw, bB, fd, ci, aY, fw, wa, aE, u, rJ, aEJ, pX, bW, Ds, K, hu, eL, cp, YL, ZH, iE, Jl, aW, bKt, aoo, az, fc, kt, g, as, cm, Qx, fT, eM, nB, iA, m, uL, in, iu, cE, sw, iB, ht, fN, n, aj, fr, ct, qN, zo, ai, eW, Vs, Bl, ft, jz, aoc, ju, cU, bKs, lG, dL, h, cgQ, bzs, mR, CN, aoX, MD, fe, kp, oU, at, it, B, Dw, bev, fM, dA, eR, fG, arQ, DV, aA, fF, zN, v, ze, uW, AO, q, ccP, aKc, eJ, RU, lg, hb, afQ, aT, ae, jf, kl, s, NE, nO, oV, cgR, Y, ev, iU, r, aZg, en, avb, cl, LV, t, Px, iK, ck, nM, iM, gX, fP, dy, EY, cgP, bt, KY, rA, ao, hS, nZ, Mb, Pt, aD, ha, rl, jh, vm, bS, oH, Gn, iJ, vN, ad, dP, nH, l, F, hd, ib, Db, ds, io, ji, uD, vl, ul, je, BO, qt, ff, rM, So, aF, iV, Z, bz, LB, es, mO, bF, bE, y, X, BA); #c1(MUC1) c2(NP_001191214) c3(mucin-1 isoform 9 precursor) c4(81114, 81115) c5(81116, 81117, 81118, 81119, 81120, 81121, 81122, 81123, 81124, 81125, 81126, 81127) c6( ) c7(oO, bM, T, aC, fR, pf, if, bKw, bB, fd, ci, aY, fw, wa, aE, u, rJ, aEJ, pX, bW, Ds, K, hu, eL, cp, YL, ZH, iE, Jl, aW, bKt, aoo, az, fc, kt, g, as, cm, Qx, fT, eM, nB, iA, m, uL, in, iu, cE, sw, iB, ht, fN, n, aj, fr, ct, qN, zo, ai, eW, Vs, Bl, ft, jz, aoc, ju, cU, bKs, lG, dL, h, cgQ, bzs, mR, CN, aoX, MD, fe, kp, oU, at, it, B, Dw, bev, fM, dA, eR, fG, ard, DV, aA, fF, zN, v, ze, uW, AO, q, ccP, aKc, eJ, RU, lg, hb, afQ, aT, ae, jf, kl, s, NE, nO, oV, cgR, Y, ev, iU, r, aZg, en, avb, cl, LV, t, Px, iK, ck, nM, iM, gX, fP, dy, EY, cgP, bt, KY, rA, ao, hS, nZ, Mb, Pt, aD, ha, rl, jh, vm, bS, oH, Gn, iJ, vN, ad, dP, nH, l, F, hd, ib, Db, ds, io, ji, uD, vl, uQ, je, BO, qt, ff, rM, So, aF, iV, Z, bz, LB, es, mO, bF, bE, y, X, BA); #c1(MUC1) c2(NP_001191218) c3(mucin-I isoform 13 precursor) c4(81128, 81129) c5(81130, 81131, 81132, 81133, 81134, 81135, 81136, 81137, 81138, 81139, 81140, 81141) c6( ) c7(oO, bM, T, aC, fR, pf, if, bKw, bB, fd, ci, aY, fw, wa, aE, u, rJ, aEJ, pX, bW, Ds, K, hu, eL, cp, YL, ZH, iE, Jl, aW, bKt, aoo, az, fc, kt, g, as, cm, Qx, fT, eM, nB, iA, m, uL, in, iu, cE, sw, iB, ht, fN, n, aj, fr, ct, qN, zo, ai, eW, Vs, Bl, ft, jz, aoc, ju, cU, bKs, lG, dL, h, cgQ, bzs, mR, CN, aoX, MD, fe, kp, oU, at, it, B, Dw, bev, fM, dA, eR, fG, arQ, DV, aA, fF, zN, v, ze, uW, AO, q, ccP, aKc, eJ, RU, lg, hb, afQ, aT, ae, jf, kl, s, NE, nO, oV, cgR, Y, ev, iU, r, aZg, en, avb, cl, LV, t, Px, iK, ck, nM, iM, gX, fP, dy, EY, cgP, bt, KY, rA, ao, hS, nZ, Mb, Pt, aD, ha, rl, jh, vm, bS, oH, Gn, iJ, vN, ad, dP, nH, l, F, hd, ib, Db, ds, io, ji, uD, vl, uQ, je, BO, qt, ff, rM, So, aF, iV, Z, bz, LB, es, mO, bF, bE, y, X, BA); #c1(MUC1) c2(NP_001191222) c3(mucin-1 isoform 17 precursor) c4(81142) c5(81143, 81144, 81145, 81146, 81147, 81148) c6( ) c7(oO, bM, T, aC, fR, pf, if, bKw, bB, fd, ci, aY, fw, wa, aE, u, rJ, aEJ, pX, bW, Ds, K, hu, eL, cp, YL, ZH, iE, Jl, aW, bKt, aoo, az, fc, kt, g, as, cm, Qx, fT, eM, nB, iA, m, uL, in, iu, cE, sw, iB, ht, fN, n, aj, fr, ct, qN, zo, ai, eW, Vs, Bl, ft, jz, aoc, ju, cU, bKs, lG, dL, h, cgQ, bzs, mR, CN, aoX, MD, fe, kp, oU, at, it, B, Dw, bev, fM, dA, eR, fG, arQ, DV, aA, fF, zN, v, ze, uW, AO, q, ccP, aKc, eJ, RU, lg, hb, afQ, aT, ae, jf, kl, s, NE, nO, oV, cgR, Y, ev, iU, r, aZg, en, avb, cl, LV, t, Px, iK, ck, nM, iM, gX, fP, dy, EY, cgP, bt, KY, rA, ao, hS, nZ, Mb, Pt, aD, ha, rl, jh, vm, bS, oH, Gn, iJ, vN, ad, dP, nH, l, F, hd, ib, Db, ds, io, ji, uD, vl, ul, je, BO, qt, ff, rM, So, aF, iV, Z, bz, LB, es, mO, bF, bE, y, X, BA); #c1(MUC1) c2(NP_001191223) c3(mucin-1 isoform 18 precursor) c4(81149) c5(81150, 81151, 81152, 81153, 81154, 81155) c6( ) c7(oO, bM, T, aC, FR, pf, if, bKw, bB, fd, ci, aY, fw, wa, aE, u, rJ, aEJ, pX, bW, Ds, K, hu, eL, cp, YL, ZH, iE, Jl, aW, bKt, aoo, az, fc, kt, g, as, cm, Qx, fT, eM, nB, iA, m, uL, in, iu, cE, sw, iB, ht, fN, n, aj, fr, ct, qN, zo, ai, eW, Vs, Bl, ft, jz, aoc, ju, cU, bKs, lG, dL, h, cgQ, bzs, mR, CN, aoX, MD, fe, kp, oU, at, it, B, Dw, bev, fM, dA, eR, fG, arQ, DV, aA, fF, zN, v, ze, uW, AO, q, ccP, aKc, eJ, RU, lg, hb, afQ, aT, ae, jf, kl, s, NE, nO, oV, cgR, Y, ev, iU, r, aZg, en, avb, cl, LV, t, Px, iK, ck, nM, iM, gX, fP, dy, EY, cgP, bt, KY, rA, ao, hS, nZ, Mb, Pt, aD, ha, rl, jh, vm, bS, oH, Gn, iJ, vN, ad, dP, nH, l, F, hd, ib, Db, ds, io, ji, uD, vl, uQ, je, BO, qt, ff, rM, So, aF, iV, Z, bz, LB, es, mO, bF, bE, y, X, BA); #c1(MUC1) c2(NP_001191224) c3(mucin-1 isoform 19 precursor) c4(81156) c5(81157, 81158, 81159, 81160, 81161, 81162) c6( ) c7(oO, bM, T, aC, fR, pf, if, bKw, bB, fd, ci, aY, fw, wa, aE, u, rJ, aEJ, pX, bW, Ds, K, hu, eL, cp, YL, ZH, iE, Jl, aW, bKt, aoo, az, fc, kt, g, as, cm, Qx, fT, eM, nB, iA, m, uL, in, iu, cE, sw, iB, ht, fN, n, aj, fr, ct, qN, zo, ai, eW, Vs, Bl, ft, jz, aoc, ju, cU, bKs, lG, dL, h, cgQ, bzs, mR, CN, aoX, MD, fe, kp, oU, at, it, B, Dw, bev, fM, dA, eR, fG, arQ, DV, aA, fF, zN, v, ze, uW, AO, q, ccP, aKc, eJ, RU, lg, hb, afQ, aT, ae, jf, kl, s, NE, nO, oV, cgR, Y, ev, iU, r, aZg, en, avb, cl, LV, t, Px, iK, ck, nM, iM, gX, fP, dy, EY, cgP, bt, KY, rA, ao, hS, nZ, Mb, Pt, aD, ha, rl, jh, vm, bS, oH, Gn, iJ, vN, ad, dP, nH, l, F, hd, ib, Db, ds, io, ji, uD, vl, uQ, je, BO, qt, ff, rM, So, aF, iV, Z, bz, LB, es, mO, bF, bE, y, X, BA); #c1(MUC1) c2(NP_001191226) c3(mucin-1 isoform 21 precursor) c4(81163) c5(81164, 81165, 81166, 81167, 81168, 81169) c6( ) c7(oO, bM, T, aC, fR, pf, if, bKw, bB, fd, ci, aY, fw, wa, aE, u, rJ, aEJ, pX, bW, Ds, K, hu, eL, cp, YL, ZH, iE, Jl, aW, bKt, aoo, az, fc, kt, g, as, cm, Qx, fT, eM, nB, iA, m, uL, in, iu, cE, sw, iB, ht, fN, n, aj, fr, ct, qN, zo, ai, eW, Vs, Bl, ft, jz, aoc, ju, cU, bKs, lG, dL, h, cgQ, bzs, mR, CN, aoX, MD, fe, kp, oU, at, it, B, Dw, bev, fM, dA, eR, fG, arQ, DV, aA, fF, zN, v, ze, uW, AO, q, ccP, aKc, eJ, RU, lg, hb, afQ, aT, ae, jf, kl, s, NE, nO, oV, cgR, Y, ev, iU, r, aZg, en, avb, cl, LV, t, Px, iK, ck, nM, iM, gX, fP, dy, EY, cgP, bt, KY, rA, ao, hS, nZ, Mb, Pt, aD, ha, rl, jh, vm, bS, oH, Gn, iJ, vN, ad, dP, nH, l, F, hd, ib, Db, ds, io, ji, uD, vl, uQ, je, BO, qt, ff, rM, So, aF, iV, Z, bz, LB, es, mQ, bF, bE, y, X, BA); #c1(MUC1) c2(NP_002447) c3(mucin-1 isoform 1 precursor) c4(128516) c5(128517, 128518, 128519, 128520, 128521, 128522) c6( ) c7(oO, bM, T, aC, fR, pf, if, bKw, bB, fd, ci, aY, fw, wa, aE, u, rJ, aEJ, pX, bW, Ds, K, hu, eL, cp, YL, ZH, iE, Jl, aW, bKt, aoo, az, fc, kt, g, as, cm, Qx, fT, eM, nB, iA, m, uL, in, iu, cE, sw, iB, ht, fN, n, aj, fr, ct, qN, zo, ai, eW, Vs, Bl, ft, jz, aoc, ju, cU, bKs, IG, dL, h, cgQ, bzs, mR, CN, aoX, MD, fe, kp, oU, at, it, B, Dw, bev, fM, dA, eR, fG, arQ, DV, aA, fF, zN, v, ze, uW, AO, q, ccP, aKc, BJ, RU, lg, hb, afQ, aT, ae, jf, kl, s, NE, nO, oV, cgR, Y, ev, iU, r, aZg, en, avb, cl, LV, t, Px, iK, ck, nM, iM, gX, fP, dy, EY, cgP, bt, KY, rA, ao, hS, nZ, Mb, Pt, aD, ha, rl, jh, vm, bS, oH, Gn, iJ, vN, ad, dP, nH, l, F, hd, ib, Db, ds, io, ji, uD, vl, ul, je, BO, qt, ff, rM, So, aF, iV, Z, bz, LB, es, mO, bF, bE, y, X, BA); #c1(MUC12) c2(NP_001157934) c3(mucin-12 precursor) c4(62882, 62883) c5(62884, 62885, 62886, 62887, 62888, 62889, 62890, 62891, 62892, 62893, 62894, 62895, 62896) c6( ) c7(IV, T, q, ccP, r); #c1(MUC13) c2(NP_149038) c3(mucin-13 precursor) c4(226882) c5(226883, 226884, 226885, 226886, 226887, 226888) c6( ) c7(T, aA, bB, cy, fe, aF, m, iV, t, ccP); #c1(MUC15) c2(NP_001128563) c3(mucin-15 isoform a) c4( ) c5(50218, 50219, 50220, 50221, 50222, 50223, 50224, 50225, 50226, 50227, 50228, 50229, 50230) c6(50231, 50232, 50233, 50234) c7(bB, bE, ccP); #c1(MUC15) c2(NP_001128564) c3(mucin-15 isoform c) c4( ) c5(50235, 50236, 50237, 50238, 50239, 50240, 50241, 50242, 50243, 50244, 50245, 50246) c6(50247, 50248, 50249, 50250) c7(bB, bE, ccP); #c1(MUC15) c2(NP_663625) c3(mucin-15 isoform b precursor) c4( ) c5(241352, 241353, 241354, 241355, 241356, 241357, 241358) c6(241359, 241360) c7(bB, bE, ccP); #c1(MUC15) c2(XP_011518210) c3(mucin-15 isoform X1) c4(312246, 312247) c5(312248, 312249) c6( ) c7(bB, bE, ccP); #c1(MUC15) c2(XP_011518211) c3(mucin-15 isoform X2) c4(312250) c5(312251, 312252) c6( ) c7(bB, bE, ccP); #c1(MUC16) c2(NP_078966) c3(mucin-16) c4(216303, 216304) c5(216305, 216306, 216307, 216308, 216309, 216310, 216311, 216312, 216313, 216314, 216315, 216316, 216317, 216318) c6( ) c7(T, kh, dA, zX, iW, rl, jh, n, aj, oH, ci, ah, fr, cd, ccP, eJ, avu, dP, av, avp, gJ, ib, aE, Db, avs, aEJ, aT, vc, hS, wt, cgl, BO, aW, ff, cr, rM, Qm, kp, en, bjl, cl, t, cM, g, apx, ar, By, amS, AM, hL, m, avr, iu, vQ); #c1(MUC17) c2(NP_001035194) c3(mucin-17 precursor) c4(32575) c5(32576, 32577, 32578, 32579, 32580, 32581, 32582, 32583, 32584, 32585, 32586, 32587) c6(32588) c7(bF, ai, T, aC, y, aE, s, iE, bE, aW, r, aT, bB, kp, ji, m, iV, t, q, ccP, de); #c1(MUC19) c2(NP_775871) c3(mucin-19 precursor) c4(250524) c5(250525, 250526, 250527, 250528, 250529, 250530, 250531, 250532, 250533, 250534) c6( ) c7(fe, ccP, iV); #c1(MUC2) c2(NP_002448) c3(mucin-2 precursor) c4(128523, 128524, 128525, 128526) c5(128527, 128528, 128529, 128530, 128531, 128532, 128533, 128534, 128535, 128536, 128537, 128538, 128539, 128540, 128541, 128542, 128543, 128544, 128545, 128546, 128547, 128548, 128549, 128550, 128551, 128552, 128553, 128554, 128555, 128556, 128557, 128558, 128559) c6( ) c7(cgT, bM, T, aC, pf, al, bB, ci, fw, aE, u, Ds, K, MO, eL, Qe, YL, iE, aW, az, hl, LZ, kt, g, eM, dc, mx, m, in, sw, NT, cd, zo, jz, cc, ju, ep, hB, YO, PX, fe, aq, kp, pm, cM, Dw, aA, tc, q, ccP, aT, cy, ae, ls, s, NE, ev, Pg, r, Ox, en, cl, t, aKe, gX, EY, fD, rA, ao, arh, Gn, jE, cgS, ad, l, ib, NS, io, vc, ji, jo, bby, WW, mu, qt, ff, rM, aF, iV, bz, bF, bE, lI, wz, y, X, BA); #c1(MUC20) c2(NP_001269435) c3(mucin-20 isoform a precursor) c4(101646, 101647) c5(101648, 101649, 101650, 101651, 101652, 101653, 101654, 101655, 101656, 101657, 101658, 101659, 101660, 101661, 101662, 101663) c6(101664, 101665, 101666, 101667) c7(ey, ccP, hL, c1); #c1(MUC20) c2(NP_065841) c3(mucin-20 isoform d precursor) c4(205801) c5(205802, 205803, 205804, 205805, 205806, 205807, 205808, 205809) c6(205810, 205811) c7(ey, ccP, hL, c1); #c1(MUC20) c2(NP_689886) c3(mucin-20 isoform b precursor) c4(244260) c5(244261, 244262, 244263, 244264, 244265, 244266, 244267, 244268) c6(244269, 244270) c7(ey, ccP, hL, cl); #c1(MUC21) c2(NP_001010909) c3(mucin-21 precursor) c4(24086, 24087, 24088) c5(24089, 24090, 24091, 24092, 24093, 24094, 24095, 24096, 24097, 24098, 24099, 24100, 24101, 24102, 24103, 24104, 24105, 24106, 24107, 24108, 24109, 24110, 24111, 24112, 24113, 24114, 24115, 24116, 24117, 24118, 24119, 24120, 24121, 24122, 24123, 24124, 24125, 24126, 24127, 24128, 24129, 24130, 24131, 24132, 24133, 24134, 24135, 24136, 24137, 24138, 24139, 24140, 24141, 24142, 24143, 24144, 24145, 24146, 24147, 24148, 24149, 24150, 24151, 24152, 24153, 24154, 24155, 24156, 24157, 24158, 24159) c6( ) c7(xS, ff, cr, eZ, hD, ci, en, gT, hH); #c1(MUC21) c2(XP_005275180) c3(mucin-21 isoform X1) c4(285606, 285607, 285608) c5( ) c6( ) c7(xS, ff, cr, eZ, hD, ci, en, gT, hH); #c1(MUC21) c2(XP_005275463) c3(mucin-21 isoform X1) c4(285610, 285611, 285612) c5( ) c6( ) c7(xS, ff, cr, eZ, HD, ci, en, gT, hH); #c1(MUC22) c2(NP_001185744) c3(mucin-22 precursor) c4(76175) c5(76176, 76177, 76178, 76179, 76180, 76181, 76182) c6( ) c7(aZL); #c1(MUC22) c2(XP_011545884) c3(mucin-22 isoform X1) c4( ) c5(344790) c6( ) c7(aZL); #c1(MUC3A) c2(NP_005951) c3(mucin-3A precursor) c4(160632) c5(160633, 160634, 160635, 160636, 160637, 160638, 160639, 160640, 160641, 160642, 160643, 160644) c6( ) c7(bF, ai, T, aC, y, s, iE, bE, aW, aT, r, bB, ci, m, iV, t, q, ccP, Qe); #c1(MUC3A) c2(XP_011514548) c3(mucin-3A isoform X1) c4(307245) c5( ) c6( ) c7(bF, ai, T, aC, y, s, iE, bE, aW, aT, r, bB, ci, m, iV, t, q, ccP, Qe); #c1(MUC3A) c2(XP_011514551) c3(mucin-3A isoform X4) c4(307246) c5( ) c6( ) c7(bF, ai, T, aC, y, s, iE, bE, aW, aT, r, bB, ci, m, iV, t, q, ccP, Qe); #c1(MUC3A) c2(XP_011514553) c3(mucin-3A isoform X6) c4( ) c5(307247, 307248, 307249, 307250, 307251, 307252, 307253) c6( ) c7(bF, ai, T, aC, y, s, iE, bE, aW, aT, r, bB, ci, m, iV, t, q, ccP, Qe); #c1(MUC4) c2(NP_004523) c3(mucin-4 isoform d precursor) c4( ) c5(147234, 147235, 147236, 147237, 147238, 147239) c6( ) c7(bM, T, Dw, aC, sw, iB, fN, aA, bB, n, oH, ci, fr, iJ, zo, ad, El, ccP, dP, aKc, eJ, iu, jz, aE, akU, rJ, pX, aT, Ds, bj, hu, ae, Qe, s, NE, Jl, aW, Y, ff, eH, nY, Ox, aq, fe, kp, aF, en, g, eF, t, LV, bz, iV, aBs, bF, bIC, QG, eM, bE, m, cE, B, ao); #c1(MUC4) c2(NP_060876) c3(mucin-4 isoform a precursor) c4(198237, 198238, 198239, 198240, 198241) c5(198242, 198243, 198244, 198245, 198246, 198247, 198248, 198249, 198250, 198251, 198252, 198253, 198254, 198255, 198256, 198257, 198258, 198259) c6( ) c7(bM, T, Dw, aC, sw, iB, fN, aA, bB, n, oH, ci, fr, iJ, zo, ad, El, ccP, dP, aKc, eJ, iu, jz, aE, akU, rJ, pX, aT, Ds, bj, hu, ae, Qe, s, NE, Jl, aW, Y, ff, eH, nY, Ox, aq, fe, kp, aF, en, g, eF, t, LV, bz, iV, aBs, bF, bIC, QG, eM, bE, m, cE, B, ao); #c1(MUC4) c2(NP_612154) c3(mucin-4 isoform e precursor) c4( ) c5(236107, 236108, 236109, 236110, 236111, 236112) c6( ) c7(bM, T, Dw, aC, sw, iB, fN, aA, bB, n, oH, ci, fr, iJ, zo, ad, El, ccP, dP, aKc, eJ, iu, jz, aE, akU, rJ, pX, aT, Ds, bj, hu, ae, Qe, s, NE, Jl, aW, Y, ff, eH, nY, Ox, aq, fe, kp, aF, en, g, eF, t, LV, bz, iV, aBs, bF, bIC, QG, eM, bE, m, cE, B, ao); #c1(MUC5AC) c2(NP_001291288) c3(mucin-5AC precursor) c4(115645, 115646) c5(115647, 115648, 115649, 115650, 115651, 115652, 115653, 115654, 115655, 115656, 115657, 115658, 115659, 115660, 115661) c6( ) c7(bM, T, aC, pf, bB, ci, fw, aE, aEJ, Ds, K, aa, iE, az, LZ, nY, g, eM, m, in, cE, aZN, sw, fN, n, BF, zo, ai, BI, cU, cgQ, YO, fe, kp, mo, cM, JM, at, ZZ, Dw, fG, aA, q, ccP, aOY, aT, cgU, yb, dl, ls, kl, s, NE, cgR, ev, r, lo, Ph, en, cl, LV, t, aKe, EY, fD, ao, Mb, aZ, aD, rl, ad, Os, bby, qt, ff, rM, aF, Sm, bz, DF, TA, bF, bE, dO, uR, X, y, iu, BA); #c1(MUC5B) c2(NP_002449) c3(mucin-58 precursor) c4(128560, 128561, 128562, 128563, 128564, 128565, 128566, 128567, 128568) c5(128569, 128570, 128571, 128572, 128573, 128574, 128575, 128576, 128577, 128578, 128579, 128580, 128581, 128582, 128583, 128584, 128585, 128586, 128587, 128588, 128589, 128590, 128591, 128592, 128593, 128594, 128595, 128596, 128597, 128598, 128599, 128600, 128601, 128602, 128603, 128604, 128605, 128606, 128607, 128608, 128609, 128610, 128611, 128612, 128613, 128614, 128615, 128616, 128617, 128618, 128619, 128620, 128621, 128622, 128623) c6( ) c7(bM, T, Dw, fG, fN, aA, bB, btu, ci, zo, ccP, ad, aE, bW, aT, h, Ds, dL, K, ae, ul, aa, ls, aW, ev, az, hQ, kt, fe, aF, g, cM, pb, zV, DF, LB, bF, eM, bE, aZL, uR, at, iu, ao); #c1(MUC6) c2(NP_005952) c3(mucin-6 precursor) c4(160645) c5(160646, 160647, 160648, 160649, 160650, 160651) c6( ) c7(T, fR, sw, aA, ci, q, ccP, ad, BI, ZZ, aE, qp, aT, ae, dl, bc, ls, s, iE, Jl, aW, qt, az, r, kt, fe, aF, g, bF, gX, iu, ao); #c1(MUC7) c2(NP_001138479) c3(mucin-7 precursor) c4(56852, 56853, 56854, 56855, 56856, 56857, 56858, 56859, 56860) c5(56861, 56862, 56863, 56864, 56865, 56866, 56867, 56868, 56869, 56870, 56871, 56872, 56873, 56874, 56875, 56876, 56877, 56878, 56879, 56880, 56881) c6( ) c7(bM, BI, Dw, qt, sp, ff, rM, Ds, dL, en, g, eL, eL, ccP); #c1(MUCL1) c2(XP_011536170) c3(mucin-like protein 1 isoform X1) c4(333477) c5(333478, 333479, 333480, 333481, 333482, 333483, 333484) c6(333485) c7(T, pf, aW, aT, AO, it); #c1(MUM1) c2(NP_116242) c3(PWWP domain-containing protein MUM1) c4(225911, 225912) c5(225913, 225914, 225915, 225916, 225917, 225918) c6( ) c7(apQ, fM, jh, fd, lo, pk); #c1(MUM1) c2(XP_011526684) c3(PWWP domain-containing protein MUM1 isoform X3) c4(320962) c5(320963, 320964, 320965, 320966, 320967, 320968) c6( ) c7(apQ, fM, jh, fd, lo, pk); #c1(MUM1) c2(XP_011526686) c3(PWWP domain-containing protein MUM1 isoform X5) c4( ) c5(320969, 320970, 320971, 320972, 320973, 320974) c6( ) c7(apQ, fM, jh, fd, lo, pk); #c1(MUM1) c2(XP_011526687) c3(PWWP domain-containing protein MUM1 isoform X6) c4(320975, 320976) c5(320977, 320978, 320979, 320980, 320981, 320982, 320983, 320984, 320985, 320986, 320987, 320988) c6( ) c7(apQ, fM, jh, fd, lo, pk); #c1(MURC) c2(NP_001018126) c3(muscle-related coiled-coil protein) c4(27445) c5(27446, 27447, 27448, 27449, 27450, 27451) c6( ) c7(Hr); #c1(MUS81) c2(XP_011543572) c3(crossover junction endonuclease MUS81 isoform X1) c4(342486) c5(342487, 342488, 342489, 342490, 342491, 342492, 342493, 342494, 342495, 342496) c6( ) c7(GI, T, os, vl, eM, dB, sw, aW, bE, ff, aT, bB, oZ, K, en, eA, V, B); #c1(MUSK) c2(NP_001159752) c3(muscle, skeletal receptor tyrosine-protein kinase isoform 2) c4(64713, 64714, 64715) c5(64716, 64717, 64718, 64719, 64720, 64721, 64722, 64723, 64724, 64725, 64726, 64727, 64728) c6( ) c7(xA, aOf, hM, aw, ha, cgV, bl, xe); #c1(MUSK) c2(NP_001159753) c3(muscle, skeletal receptor tyrosine-protein kinase isoform 3) c4( ) c5(64729, 64730, 64731, 64732, 64733, 64734, 64735, 64736, 64737, 64738) c6( ) c7(xA, aOf, hM, aw, ha, cgV, bl, xe); #c1(MUSK) c2(NP_005583) c3(muscle, skeletal receptor tyrosine-protein kinase isoform 1) c4(157670, 157671, 157672) c5(157673, 157674, 157675, 157676, 157677, 157678, 157679, 157680, 157681, 157682, 157683, 157684, 157685, 157686, 157687, 157688, 157689, 157690) c6( ) c7(xA, aOf, hM, aw, ha, cgV, bl, xe); #c1(MUSK) c2(XP_005252053) c3(muscle, skeletal receptor tyrosine-protein kinase isoform X4) c4(271504) c5(271505, 271506, 271507, 271508, 271509, 271510) c6( ) c7(xA, aOf, hM, aw, ha, cgV, bl, xe); #c1(MUSK) c2(XP_011517009) c3(muscle, skeletal receptor tyrosine-protein kinase isoform X1) c4(310499) c5( ) c6( ) c7(xA, aOf, hM, aw, ha, cgV, bl, xe); #c1(MUSK) c2(XP_011517010) c3(muscle, skeletal receptor tyrosine-protein kinase isoform X5) c4( ) c5(310500, 310501, 310502, 310503, 310504, 310505, 310506, 310507, 310508) c6( ) c7(xA, aOf, hM, aw, ha, cgV, bl, xe); #c1(MUT) c2(NP_000246) c3(methylmalonyl-CoA mutase, mitochondrial precursor) c4(5597, 5598, 5599, 5600) c5(5601, 5602, 5603, 5604, 5605, 5606, 5607, 5608, 5609, 5610, 5611, 5612, 5613, 5614, 5615, 5616, 5617, 5618, 5619, 5620, 5621, 5622, 5623, 5624, 5625, 5626, 5627, 5628, 5629, 5630, 5631, 5632, 5633, 5634, 5635, 5636, 5637, 5638, 5639, 5640, 5641, 5642, 5643, 5644, 5645, 5646, 5647, 5648, 5649, 5650, 5651, 5652, 5653, 5654, 5655, 5656, 5657, 5658, 5659, 5660, 5661, 5662, 5663, 5664, 5665, 5666, 5667, 5668, 5669, 5670, 5671, 5672, 5673, 5674, 5675, 5676, 5677, 5678, 5679, 5680, 5681, 5682, 5683, 5684, 5685, 5686, 5687, 5688, 5689, 5690, 5691, 5692, 5693, 5694, 5695, 5696, 5697, 5698, 5699, 5700, 5701, 5702, 5703, 5704) c6(5705, 5706, 5707, 5708, 5709) c7(aHq, T, if, aip, bZ, v, j, qN, q, ad, ip, cgY, hd, cc, M, aT, h, r, r, dq, cgW, pb, dr, mO, bP, eM, nB, cgX); #c1(MUTYH) c2(NP_001280119) c3(A/G-specific adenine DNA glycosylase isoform 6) c4(111333) c5(111334, 111335, 111336, 111337, 111338, (11339, 111340) c6( ) c7(bL, T, eR, eX, NT, aA, bB, aj, ci, bX, aY, V, q, J, ad, eA, ip, jz, aE, Ok, aT, eV, auS, YB, dL, K, ae, Qe, B, BO, s, dH, aW, Y, Yd, ff, r, aq, Kf, aF, en, cl, g, bz, bF, cgZ, rR, W, dB, bE, gX, hL, aMy, k, YS, X, rk); #c1(MUTYH) c2(NP_001280120) c3(A/G-specific adenine DNA glycosylase isoform 7) c4(111341) c5(111342, 111343, 111344, 111345, 111346, 111347, 111348, 111349, 111350, 111351, 111352, 111353, 111354) c6( ) c7(bL, T, eR, ex, NT, aA, bB, aj, ci, bX, aY, V, q, J, ad, eA, ip, jz, aE, Qk, aT, eV, auS, YB, dL, K, ae, Qe, B, BO, s, dH, aW, Y, Yd, ff, r, aq, Kf, aF, en, cl, g, bz, bF, cgZ, rR, W, dB, bE, gX, hL, aMy, k, YS, X, rk); #c1(MUTYH) c2(XP_011539799) c3(A/G-specific adenine DNA glycosylase isoform X2) c4(338026) c5( ) c6( ) c7(bL, T, eR, eX, NT, aA, bB, aj, ci, bX, aY, V, q, J, ad, eA, ip, jz, aE, Qk, aT, eV, auS, YB, dL, K, ae, Qe, B, BO, s, dH, aW, Y, Yd, ff, r, aq, Kf, aF, en, cl, g, bz, bF, cgZ, rR, W, dB, bE, gX, hL, aMy, k, YS, X, rk); #c1(MUTYH) c2(XP_011539809) c3(A/G-specific adenine DNA glycosylase isoform X6) c4( ) c5(338027, 338028, 338029, 338030, 338031, 338032) c6( ) c7(bL, T, eR, eX, NT, aA, bB, aj, ci, bX, aY, V, q, J, ad, eA, ip, jz, aE, Qk, aT, eV, auS, YB, dL, K, ae, Qe, B, BO, s, dH, aW, Y, Yd, ff, r, aq, Kf, aF, en, cl, g, bz, bF, cgZ, rR, W, dB, bE, gX, hL, aMy, k, YS, X, rk); #c1(MUTYH) c2(XP_011539810) c3(A/G-specific adenine DNA glycosylase isoform X7) c4( ) c5(338033, 338034, 338035, 338036, 338037, 338038) c6( ) c7(bL, T, eR, eX, NT, aA, bB, aj, ci, bX, aY, V, q, J, ad, eA, ip, jz, aE, Qk, aT, eV, auS, YB, dL, K, ae, Qe, B, BO, s, dH, aW, Y, Yd, ff, r, aq, Kf, aF, en, cl, g, bz, bF, cgZ, rR, W, dB, bE, gX, hL, aMy, k, YS, X, rk); #c1(MVB12B) c2(NP_001011703) c3(multivesicular body subunit 128 isoform 2) c4( ) c5(24747, 24748, 24749, 24750, 24751, 24752, 24753, 24754, 24755, 24756, 24757, 24758) c6( ) c7(dw, Na, Nc); #c1(MVB12B) c2(NP_258257) c3(multivesicular body subunit 12B isoform 1) c4(228676) c5(228677, 228678, 228679, 228680, 228681, 228682) c6( ) c7(dw, Na, Nc); #c1(MVB12B) c2(XP_005252354) c3(multivesicular body subunit 12B isoform X5) c4(271668, 271669, 271670) c5(271671, 271672, 271673, 271674, 271675, 271676, 271677, 271678, 271679, 271680, 271681, 271682) c6( ) c7(dw, Na, Nc); #c1(MVB12B) c2(XP_011517479) c3 (multivesicular body subunit 12B isoform X3) c4(311218) c5( ) c6( ) c7(dw, Na, Nc); #c1(MVD) c2(NP_002452) c3(diphosphomevalonate decarboxylase) c4(128633, 128634) c5(128635, 128636, 128637, 128638, 128639, 128640, 128641) c6( ) c7(T, qb, yS, bgQ, qx, n, aj, j, aqf, eA, J, oT, hd, py, jZ, dV, aT, dz, hP, pq, pl, aeR, iL, et, aW, Ls, ff, kD, en, pm, t, bw, yR, ck, pc, nB, pg, m, bt, B); #c1(MVD) c2(XP_011521391) c3(diphosphomevalonate decarboxylase isoform X4) c4( ) c5(316207, 316208, 316209, 316210, 316211) c6( ) c7(T, qb, yS, bgQ, qx, n, aj, j, aqf, eA, J, oT, hd, py, jZ, dV, aT, dz, hP, pq, pl, aeR, iU, et, aW, Ls, ff, kD, en, pm, t, bw, yR, ck, pc, nB, pg, m, bt, B); #c1(MVK) c2(NP_0012881 fl) c3(mevalonate kinase isoform b) c4(113538, 113539) c5(113540, 113541, 113542, 113543, 113544, 113545, 113546, 113547, 113548, 113549, 113550, 113551, 113552, 113553, 113554, 113555) c6( ) c7(bL, T, bzE, amj, agH, nA, aPb, q, hX, cF, NY, aT, dQ, hH, NX, ih, aLJ, r, aq, bQL, cm, nC, dr, mO, ky, cO, cha, R, i, bt, ra, B); #c1(MVK) c2(XP_011536674) c3(mevalonate kinase isoform X1) c4(334162, 334163, 334164) c5(334165, 334166, 334167, 334168, 334169, 334170, 334171, 334172, 334173, 334174, 334175, 334176, 334177, 334178, 334179, 334180, 334181, 334182) c6( ) c7(bL, T, bzE, amj, agH, nA, aPb, q, hX, cF, NY, aT, dQ, hH, NX, ih, aLJ, r, aq, bQL, cm, nC, dr, mO, ky, cO, cha, R, i, bt, ra, B); #c1(MVP) c2(NP_001280133) c3(major vault protein isoform 2) c4( ) c5(111367) c6( ) c7(T, wJ, if, aw, ha, jx, jh, fd, bB, j, ci, qN, fr, hF, eA, er, x, ft, xc, kH, aT, cy, h, K, dQ, af, Lg, d, B, je, aW, eH, nK, aq, lo, kp, as, cl, eF, t, cm, bz, QA, iK, bw, ck, iM, eM, dB, bE, m, k, bt, gD, KG); #c1(MVP) c2(NP_001280134) c3(major vault protein isoform 3) c4( ) c5(111368, 111369) c6( ) c7(T, wJ, if, aw, ha, jx, jh, fd, bB, j, ci, qN, fr, hF, eA, er, x, ft, xc, kH, aT, cy, h, K, dQ, af, Lg, d, B, je, aW, eH, nK, aq, lo, kp, as, cl, eF, t, cm, bz, QA, iK, bw, ck, iM, eM, dB, bE, m, k, bt, gD, KG); #c1(MVP) c2(NP_059447) c3(major vault protein isoform 1) c4(193312) c5(193313, 193314, 193315, 193316, 193317, 193318, 193319, 193320, 193321, 193322) c6( ) c7(T, wJ, if, aw, ha, jx, jh, fd, bB, j, ci, qN, fr, hF, eA, er, x, ft, xc, kH, aT, cy, h, K, dQ, af, Lg, d, B, je, aW, eH, nk, aq, lo, kp, as, cl, eF, t, cm, bz, QA, iK, bw, ck, iM, eM, dB, bE, m, k, bt, gD, KG); #c1(MXI) c2(XP_005261039) c3(interferon-induced GTP-binding protein Mx1 isoform X1) c4(277104, 277105, 277106, 277107, 277108) c5(277109, 277110, 277111, 277112, 277113, 277114, 277115, 277116, 277117, 277118, 277119, 277120, 277121, 277122, 277123, 277124, 277125, 277126, 277127, 277128, 277129, 277130, 277131, 277132) c6( ) c7(fR, al, jx, aA, oZ, j, jE, V, eW, aFu, hX, F, ds, h, dL, kU, af, afu, qZ, lh, wc, sP, eZ, tx, aHU, lo, CO, Fq, t, f, bNw, pb, atM, aiM, at, eo, bt, Pr, B, chb, L); #c1(MXI) c2(XP_011527871) c3(interferon-induced GTP-binding protein Mx1 isoform X2) c4(322918) c5( ) c6( ) c7(fR, al, jx, aA, oZ, j, jE, V, eW, aFu, hX, F, ds, h, dL, kU, af, afu, qZ, lh, wc, sP, eZ, tx, aHU, lo, CQ, Fq, t, f, bNw, pb, atM, aiM, at, eo, bt, Pr, B, chb, L); #c1(MXI) c2(XP_011527872) c3(interferon-induced GTP-binding protein Mx1 isoform X3) c4(322919) c5(322920, 322921, 322922, 322923, 322924, 322925, 322926, 322927, 322928, 322929, 322930, 322931, 322932, 322933, 322934, 322935, 322936, 322937, 322938, 322939, 322940, 322941, 322942, 322943, 322944, 322945, 322946, 322947) c6( ) c7(fR, al, jx, aA, oZ, j, jE, V, eW, aFu, hX, F, ds, h, dL, kU, af, afu, qZ, lh, wc, sP, eZ, tx, aHU, lo, CO, Fq, t, f, bNw, pb, atM, aiM, at, eo, bt, Pr, B, chb, L); #c1(MX2) c2(XP_011527874) c3(interferon-induced GTP-binding protein Mx2 isoform X1) c4(322948) c5(322949, 322950, 322951, 322952, 322953, 322954, 322955, 322956, 322957, 322958) c6( ) c7(cF, ds, Y, jx, h, iu); #c1(MX2) c2(XP_011527875) c3(interferon-induced GTP-binding protein Mx2 isoform X2) c4(322959, 322960, 322961, 322962, 322963) c5(322964, 322965, 322966, 322967, 322968, 322969, 322970, 322971, 322972, 322973, 322974, 322975, 322976, 322977, 322978, 322979, 322980, 322981, 322982, 322983, 322984, 322985, 322986, 322987, 322988) c6( ) c7(cF, ds, Y, jx, h, iu); #c1(MXD1) c2(NP_001189443) c3(max dimerization protein 1 isoform 3) c4(79387) c5(79388, 79389, 79390, 79391, 79392, 79393) c6( ) c7(T, pf, jx, eX, bB, fr, ci, hF, q, br, ad, eA, cc, F, aT, ta, eV, ae, pi, jf, d, s, aW, Y, oV, fJ, r, aq, aF, g, as, iR, cm, bw, bz, bE, cj, m, bt, B); #c1(MXD1) c2(NP_002348) c3(max dimerization protein 1 isoform 1) c4(127705) c5(127706, 127707, 127708, 127709, 127710, 127711, 127712) c6( ) c7(T, pf, jx, eX, bB, fr, ci, hF, q, br, ad, eA, cc, F, aT, ta, eV, ae, pi, jf, d, s, aW, Y, oV, fJ, r, aq, aF, g, as, iR, cm, bw, bz, bE, cj, m, bt, B); #c1(MXD3) c2(NP_001136407) c3(max dimerization protein 3 isoform b) c4(54926) c5(54927, 54928, 54929, 54930, 54931, 54932, 54933, 54934, 54935, 54936, 54937, 54938) c6( ) c7(dB, T, jf); #c1(MXD3) c2(NP_112590) c3(max dimerization protein 3 isoform a) c4(221498, 221499, 221500) c5(221501, 221502, 221503, 221504, 221505, 221506, 221507, 221508, 221509, 221510, 221511, 221512, 221513, 221514, 221515, 221516, 221517) c6( ) c7(dB, T, jf); #c1(MXD4) c2(NP_006445) c3(max dimerization protein 4) c4(165278, 165279) c5(165280, 165281, 165282, 165283, 165284, 165285, 165286, 165287, 165288, 165289, 165290, 165291, 165292) c6( ) c7(iC, d, dB, h, Nf, as, Fc, pi); #c1(MXI1) c2(NP_001008541) c3(max-interacting protein 1 isoform c) c4(23332) c5(23333, 23334, 23335, 23336, 23337) c6( ) c7(G, T, id, atY, ht, aA, n, ci, pz, hF, V, q, ad, eA, eJ, av, no, py, arV, aT, dL, ae, eL, jf, d, Ub, aW, Y, eH, aq, aF, g, as, eF, t, cm, bz, iV, es, byl, bP, eM, iA, dB, bE, iT, wB, cj, dy, m, Hb, B); #c1(MXI1) c2(NP_005953) c3(max-interacting protein 1 isoform a) c4(160652, 160653, 160654) c5(160655, 160656, 160657, 160658, 160659, 160660, 160661, 160662, 160663, 160664, 160665, 160666, 160667, 160668, 160669, 160670, 160671, 160672, 160673, 160674, 160675, 160676, 160677, 160678, 160679, 160680, 160681, 160682, 160683, 160684, 160685, 160686, 160687, 160688, 160689, 160690, 160691, 160692) c6( ) c7(G, T, id, atY, ht, aA, n, ci, pz, hF, V, q, ad, eA, eJ, av, no, py, arV, aT, dL, ae, eL, jf, d, Ub, aW, Y, eH, aq, aF, g, as, eF, t, cm, bz, iV, es, byl, bP, eM, iA, dB, bE, iT, wB, cj, dy, m, Hb, B); #c1(MXI1) c2(NP_569157) c3(max-interacting protein 1 isoform b) c4(233705, 233706, 233707) c5(233708, 233709, 233710, 233711, 233712, 233713, 233714, 233715, 233716, 233717, 233718, 233719) c6( ) c7(G, T, id, atY, ht, aA, n, ci, pz, hF, V, q, ad, eA, eJ, av, no, py, arV, aT, dL, ae, eL, jf, d, Ub, aW, Y, eH, aq, aF, g, as, eF, t, cm, bz, iV, es, byl, bP, eM, iA, dB, bE, iT, wB, cj, dy, m, Hb, B); #c1(MXRA5) c2(NP_056234) c3(matrix-remodeling-associated protein 5 precursor) c4(186622) c5(186623, 186624, 186625, 186626, 186627, 186628, 186629, 186630, 186631, 186632, 186633, 186634) c6(186635, 186636, 186637, 186638) c7(T, bz, eM); #c1(MXRA5) c2(XP_005274542) c3(matrix-remodeling-associated protein 5 isoform X1) c4( ) c5(285150, 285151) c6( ) c7(T, bz, eM); #c1(MYADM) c2(NP_001277119) c3(myeloid-associated differentiation marker) c4(109049, 109050, 109051, 109052, 109053, 109054) c5(109055, 109056, 109057, 109058, 109059, 109060, 109061, 109062, 109063, 109064, 109065, 109066, 109067, 109068, 109069, 109070, 109071, 109072, 109073, 109074, 109075, 109076, 109077, 109078, 109079, 109080, 109081, 109082, 109083, 109084, 109085, 109086, 109087, 109088, 109089, 109090, 109091, 109092, 109093, 109094, 109095, 109096, 109097, 109098, 109099, 109100, 109101, 109102, 109103, 109104, 109105, 109106) c6( ) c7(B); #c1(MYB) c2(NP_001123644) c3(transcriptional activator Myb isoform 3) c4(47939) c5(47940, 47941, 47942, 47943, 47944, 47945) c6( ) c7(bM, T, qb, atC, aw, chc, jh, cz, bB, rf, v, j, ci, pM, hF, AO, q, br, eA, x, pN, kl, qK, dV, kH, aT, dz, hn, K, in, qw, yo, Nr, d, je, iU, et, aW, Y, r, po, bow, as, hf, cm, bw, pb, Hp, Nq, ck, BU, iM, Dy, awC, qe, bE, eM, iT, z, mg, pg, k, pw, bt, X); #c1(MYB) c2(NP_001123645) c3(transcriptional activator Myb isoform 1) c4(47946, 47947, 47948) c5(47949, 47950, 47951, 47952, 47953, 47954) c6( ) c7(bM, T, qb, atC, aw, chc, jh, cz, bB, rf, v, j, ci, pM, hF, AO, q, br, eA, x, pN, kl, qK, dV, kH, aT, dz, hn, K, in, qw, yo, Nr, d, je, iU, et, aW, Y, r, po, bow, as, hf, cm, bw, pb, Hp, Nq, ck, BU, iM, Dy, awC, qe, bE, eM, iT, z, mg, pg, k, pw, bt, X); #c1(MYB) c2(NP_001155129) c3(transcriptional activator Myb isoform 5) c4( ) c5(61447, 61448, 61449, 61450, 61451, 61452, 61453, 61454, 61455, 61456) c6( ) c7(bM, T, qb, atC, aw, chc, jh, cz, bB, rf, v, j, ci, pM, hF, AO, q, br, eA, x, pN, kl, qK, dV, kH, aT, dz, hn, K, in, qw, yo, Nr, d, je, iU, et, aW, Y, r, po, bow, as, hf, cm, bw, pb, Hp, Nq, ck, BU, iM, Dy, awC, qe, bE, eM, iT, z, mg, pg, k, pw, bt, X); #c1(MYB) c2(NP_001155132) c3(transcriptional activator Myb isoform 8) c4(61457) c5(61458, 61459, 61460, 61461, 61462, 61463) c6( ) c7(bM, T, qb, atC, aw, chc, jh, cz, bB, rf, v, j, ci, pM, hF, AO, q, br, eA, x, pN, kl, qK, dV, kH, aT, dz, hn, K, in, qw, yo, Nr, d, je, iU, et, aW, Y, r, po, bow, as, hf, cm, bw, pb, Hp, Nq, ck, BU, iM, Dy, awC, qe, bE, eM, iT, z, mg, pg, k, pw, bt, X); #c1(MYB) c2(NP_005366) c3(transcriptional activator Myb isoform 2) c4(155466, 155467, 155468) c5(155469, 155470, 155471, 155472, 155473, 155474, 155475, 155476, 155477, 155478, 155479, 155480, 155481, 155482, 155483, 155484, 155485, 155486, 155487, 155488, 155489) c6( ) c7(bM, T, qb, atC, aw, chc, jh, cz, bB, rf, v, j, ci, pM, hF, AO, q, br, eA, x, pN, kl, qK, dV, kH, aT, dz, hn, K, in, qw, yo, Nr, d, je, iU, et, aW, Y, r, po, bow, as, hf, cm, bw, pb, Hp, Nq, ck, BU, iM, Dy, awC, qe, bE, eM, iT, z, mg, pg, k, pw, bt, X); #c1(MYBBP1A) c2(NP_001099008) c3(myb-binding protein (A isoform 1) c4(41616, 41617) c5(41618, 41619, 41620, 41621, 41622, 41623, 41624, 41625, 41626, 41627, 41628, 41629) c6( ) c7(gj, T, ig, aW, aT, ae, ad); #c1(MYBBP1A) c2(NP_055335) c3(myb-binding protein (A isoform 2) c4(180579) c5(180580, 180581, 180582, 180583, 180584, 180585, 180586) c6( ) c7(gj, T, ig, aW, aT, ae, ad); #c1(MYBL1) c2(NP_001073885) c3(myb-related protein A isoform 1) c4(36295) c5(36296, 36297, 36298, 36299, 36300, 36301, 36302, 36303, 36304, 36305, 36306, 36307, 36308) c6( ) c7(jh, kl, eA, qK); #c1(MYBL1) c2(NP_001138227) c3(myb-related protein A isoform 2) c4(56055) c5(56056, 56057, 56058, 56059, 56060, 56061) c6( ) c7(jh, kl, eA, qK); #c1(MYBL1) c2(XP_011515835) c3(myb-related protein A isoform X1) c4(308808) c5( ) c6( ) c7(jh, kl, eA, qK); #c1(MYBL2) c2(NP_001265539) c3(myb-related protein B isoform 2) c4(98772) c5(98773, 98774, 98775, 98776, 98777, 98778) c6( ) c7(T, bP, aW, atC, if, cj, aT, at, fd, dL, lR, K, qN, pb, bw, cm, bt, B); #c1(MYBL2) c2(NP_002457) c3(myb-related protein B isoform 1) c4(128649) c5(128650, 128651, 128652, 128653, 128654, 128655, 128656) c6( ) c7(T, bP, aW, atC, if, cj, aT, at, fd, dL, lR, K, qN, pb, bw, cm, bt, B); #c1(MYBPC1) c2(NP_001241647) c3(myosin-binding protein C, slow-type isoform 5) c4(88945) c5(88946, 88947, 88948, 88949, 88950, 88951) c6( ) c7(chd, vc, mx, oj, che); #c1(MYBPC1) c2(NP_001241648) c3(myosin-binding protein C, slow-type isoform 6) c4(88952) c5(88953, 88954, 88955, 88956, 88957, 88958) c6( ) c7(chd, vc, mx, oj, che); #c1(MYBPC1) c2(NP_001241650) c3(myosin-binding protein C, slow-type isoform 8) c4(88959) c5(88960, 88961, 88962, 88963, 88964, 88965) c6( ) c7(chd, vc, mx, oj, che); #c1(MYBPC1) c2(NP_001241651) c3(myosin-binding protein C, slow-type isoform 9) c4(88966) c5(88967, 88968, 88969, 88970, 88971, 88972) c6( ) c7(chd, vc, mx, oj, che); #c1(MYBPC1) c2(NP_001241652) c3(myosin-binding protein C, slow-type isoform 10) c4(88973) c5(88974, 88975, 88976, 88977, 88978, 88979) c6( ) c7(chd, vc, mx, oj, che); #c1(MYBPC1) c2(NP_002456) c3(myosin-binding protein C, slow-type isoform 1) c4(128642) c5(128643, 128644, 128645, 128646, 128647, 128648) c6( ) c7(chd, vc, mx, oj, che); #c1(MYBPC1) c2(NP_996555) c3(myosin-binding protein C, slow-type isoform 2) c4(264284) c5(264285, 264286, 264287, 264288, 264289, 264290) c6( ) c7(chd, vc, mx, oj, che); #c1(MYBPC1) c2(NP_996556) c3(myosin-binding protein C, slow-type isoform 3) c4(264291) c5(264292, 264293, 264294, 264295, 264296, 264297, 264298, 264299, 264300, 264301) c6( ) c7(chd, vc, mx, oj, che); #c1(MYBPC1) c2(NP_996557) c3(myosin-binding protein C, slow-type isoform 4) c4(264302) c5(264303, 264304, 264305, 264306, 264307, 264308) c6( ) c7(chd, vc, mx, oj, che); #c1(MYBPC1) c2(XP_006719473) c3(myosin-binding protein C, slow-type isoform X6) c4(292504) c5(292505, 292506, 292507, 292508, 292509, 292510) c6( ) c7(chd, vc, mx, oj, che); #c1(MYBPC1) c2(XP_006719474) c3(myosin-binding protein C, slow-type isoform X8) c4(292511) c5(292512, 292513, 292514, 292515, 292516, 292517) c6( ) c7(chd, vc, mx, oj, che); #c1(MYBPC2) c2(NP_004524) c3(myosin-binding protein C, fast-type) c4(147240) c5(147241, 147242, 147243, 147244, 147245, 147246) c6( ) c7(vc, cc, mx); #c1(MYBPC3) c2(NP_000247) c3(myosin-binding protein C, cardiac-type) c4(5710, 5711, 5712) c5(5713, 5714, 5715, 5716, 5717, 5718, 5719, 5720, 5721, 5722, 5723, 5724, 5725, 5726, 5727, 5728, 5729, 5730) c6( ) c7(cc, cl, mR, ec, mr, chf, chh, chi, oj, ld, gZ, cd, vc, mx, j, wl, mo, aYo, rz, chg, gq); #c1(MYBPH) c2(NP_004988) c3(myosin-binding protein H) c4(151765) c5(151766, 151767, 151768, 151769, 151770, 151771) c6( ) c7(LB, T, iM, qZ, je, UZ, cz, yu, Pp, CQ, g, iu); #c1(MYC) c2(NP_002458) c3(myc proto-oncogene protein) c4(128657, 128658, 128659, 128660, 128661, 128662, 128663, 128664, 128665) c5(128666, 128667, 128668, 128669, 128670, 128671, 128672, 128673, 128674, 128675, 128676, 128677, 128678, 128679, 128680, 128681, 128682, 128683, 128684, 128685, 128686, 128687, 128688, 128689, 128690, 128691, 128692, 128693, 128694, 128695, 128696, 128697, 128698, 128699, 128700, 128701, 128702, 128703, 128704, 128705, 128706, 128707, 128708, 128709, 128710, 128711, 128712, 128713, 128714, 128715, 128716, 128717, 128718, 128719, 128720, 128721, 128722, 128723, 128724, 128725, 128726, 128727, 128728, 128729, 128730, 128731, 128732, 128733, 128734, 128735, 128736, 128737, 128738, 128739, 128740, 128741, 128742, 128743, 128744, 128745, 128746, 128747, 128748, 128749, 128750, 128751, 128752, 128753, 128754, 128755, 128756, 128757, 128758, 128759, 128760, 128761, 128762, 128763, 128764, 128765, 128766, 128767, 128768, 128769, 128770, 128771, 128772, 128773, 128774, 128775, 128776, 128777, 128778, 128779, 128780, 128781, 128782, 128783, 128784, 128785, 128786, 128787, 128788, 128789, 128790, 128791, 128792, 128793, 128794, 128795, 128796, 128797, 128798, 128799, 128800, 128801, 128802, 128803, 128804, 128805, 128806, 128807, 128808, 128809, 128810, 128811, 128812, 128813, 128814, 128815, 128816, 128817, 128818, 128819, 128820, 128821, 128822, 128823, 128824, 128825, 128826, 128827, 128828, 128829, 128830, 128831, 128832, 128833, 128834, 128835, 128836, 128837, 128838, 128839, 128840, 128841, 128842, 128843, 128844, 128845, 128846, 128847, 128848, 128849, 128850, 128851, 128852, 128853, 128854, 128855, 128856, 128857, 128858, 128859, 128860, 128861, 128862, 128863, 128864, 128865, 128866, 128867, 128868, 128869, 128870, 128871, 128872, 128873, 128874, 128875, 128876, 128877, 128878, 128879, 128880, 128881, 128882, 128883, 128884, 128885, 128886, 128887, 128888, 128889, 128890, 128891, 128892, 128893, 128894, 128895, 128896, 128897, 128898, 128899, 128900, 128901, 128902, 128903, 128904, 128905, 128906, 128907, 128908, 128909, 128910, 128911, 128912, 128913, 128914, 128915, 128916, 128917, 128918, 128919, 128920, 128921, 128922, 128923, 128924, 128925, 128926, 128927, 128928, 128929, 128930, 128931, 128932, 128933, 128934, 128935, 128936, 128937, 128938, 128939, 128940, 128941, 128942, 128943, 128944, 128945, 128946, 128947, 128948, 128949, 128950, 128951, 128952, 128953, 128954, 128955, 128956, 128957, 128958, 128959, 128960, 128961, 128962, 128963, 128964, 128965, 128966, 128967, 128968, 128969, 128970, 128971, 128972, 128973, 128974, 128975, 128976, 128977, 128978, 128979, 128980, 128981, 128982, 128983, 128984, 128985, 128986, 128987, 128988, 128989, 128990, 128991, 128992, 128993, 128994, 128995, 128996, 128997, 128998, 128999, 129000, 129001, 129002, 129003, 129004, 129005, 129006, 129007, 129008, 129009, 129010, 129011, 129012, 129013) c6(129014, 129015) c7(uB, G, T, aC, fR, ya, pf, ea, if, al, WV, fd, bB, aeu, lR, ci, ip, chj, oB, bv, x, Na, aE, SV, Vv, Ou, K, hu, pk, eL, aiJ, cp, Qe, yo, Jl, aW, az, XV, LZ, fc, CT, Qm, g, as, eF, zE, cm, bw, ou, y, eM, nB, iA, iT, wB, cj, cX, k, m, gn, ym, Xu, cho, avU, iu, dD, ajm, lT, zt, ht, aDT, n, aj, fr, qN, rP, JW, asS, br, bao, BI, ft, jz, pe, TK, Nn, zf, kH, h, eV, hn, dL, Dz, hB, on, KV, Df, dx, pI, bsk, OV, A, aoX, MD, ks, aq, apk, kp, Hu, aMJ, yq, mo, iN, op, apa, kT, iO, YE, bP, asl, dB, Qu, Vy, chI, hL, at, SZ, qF, gg, WL, it, B, ZZ, fM, jp, jx, eX, fZ, aA, ol, hz, v, j, il, AO, pd, q, J, qH, eA, cdV, mP, lg, nv, hW, aT, rO, dz, Nc, j, bq, avt, ae, af, bA, jf, iF, qw, wr, s, vT, NE, oV, Y, ev, nO, iI, xR, r, eS, en, aom, hf, t, ho, cl, pb, iK, Cv, zJ, ck, nM, kP, Dy, iM, iI, au, aor, Da, np, ij, bt, rA, vi, kh, id, LX, iW, jh, bs, KO, eq, chm, hF, ad, hX, av, hd, aop, py, oz, Ot, zK, lC, io, hD, vc, nJ, Yi, mu, azH, d, bug, chk, Ub, et, qt, je, ff, eH, chn, aeV, Xj, aF, iR, adj, KM, bz, dr, bF, es, eU, amS, pc, bE, aNL, Lb, bC, adg, hE, aed, KS, X, EB); #c1(MYCBP2) c2(NP_055872) c3(E3 ubiquitin-protein ligase MYCBP2) c4(184054, 184055) c5(184056, 184057, 184058, 184059, 184060, 184061, 184062, 184063, 184064, 184065, 184066, 184067, 184068, 184069, 184070) c6( ) c7(ck, bv, av, eM, dB, aW, aT, n, dG, v, g, cm, bjQ, bz, eA, uQ); #c1(MYCBP2) c2(XP_006719846) c3(E3 ubiquitin-protein ligase MYCBP2 isoform X5) c4(293030) c5(293031, 293032, 293033, 293034, 293035, 293036) c6( ) c7(ck, bv, av, eM, dB, aW, aT, n, dG, v, g, cm, bjQ, bz, eA, uQ); #c1(MYCBP2) c2(XP_011533307) c3(E3 ubiquitin-protein ligase MYCBP2 isoform X14) c4( ) c5(329461, 329462) c6( ) c7(ck, bv, av, eM, dB, aW, aT, n, dG, v, g, cm, bjQ, bz, eA, ul); #c1(MYCBPAP) c2(NP_115509) c3(MYCBP-associated protein) c4(222983, 222984) c5(222985, 222986, 222987, 222988, 222989, 222990, 222991, 222992, 222993, 222994, 222995, 222996) c6( ) c7(aW, T, iK, aT); #c1(MYCN) c2(NP_005369) c3(N-myc proto-oncogene protein isoform 1) c4(155490, 155491) c5(155492, 155493, 155494, 155495, 155496, 155497, 155498, 155499, 155500, 155501, 155502, 155503, 155504) c6( ) c7(T, Tb, ya, aAx, bB, ci, bNe, aJa, ip, bv, Fo, pX, SV, MU, Ou, K, aJu, eL, Jl, aW, g, eF, cm, eM, nB, cj, m, Xu, chp, qy, n, qN, b, ft, TK, hn, dL, on, dx, pI, awV, PX, aq, chq, Hu, QK, chs, Ld, bP, asl, dB, eo, B, fM, dA, oe, aA, V, q, er, qH, eA, aAF, nv, afQ, qK, ahI, aT, ae, jf, iF, Uf, Qr, Y, r, bU, t, ck, chr, nG, azk, bt, jh, oH, iJ, ad, iC, Tg, hd, iY, hP, iL, d, aF, bz, es, bkh, i, hE, y); #c1(MYCT1) c2(NP_079383) c3(myc target protein 1) c4(217855, 217856) c5(217857, 217858, 217859, 217860, 217861, 217862, 217863, 217864, 217865, 217866, 217867, 217868) c6( ) c7(Ej, T, ci, aA); #c1(MYD88) c2(NP_001166037) c3(myeloid differentiation primary response protein MyD88 isoform 5) c4(68096) c5(68097, 68098, 68099, 68100, 68101, 68102) c6( ) c7(oO, bM, T, fR, ea, fd, bB, ci, xE, vV, YX, Bc, dV, bc, hm, LC, aW, bNq, az, ml, aw, zC, bw, dc, ki, cj, Zn, m, aNn, dD, n, aj, BF, uE, JX, cU, h, ey, Og, CQ, Ca, YF, adz, hq, ar, dC, eD, fL, B, Dw, fM, lk, jx, v, j, C, pd, q, J, mP, Cc, WY, jZ, rS, aT, cy, le, r, lo, t, Cv, zJ, baw, ig, hc, cht, wR, EY, fD, zA, bTc, aDj, azW, jh, eq, atK, mC, hX, yt, ds, LA, ji, NM, cH, Of, eZ, dN, aoF, gE, hp, uF, iV, aDH, bE, gZ, ei, y); #c1(MYD88) c2(NP_001166038) c3(myeloid differentiation primary response protein MyD88 isoform 1) c4(68103) c5(68104, 68105, 68106, 68107, 68108, 68109) c6( ) c7(oO, bM, T, fR, ea, fd, bB, ci, xE, vV, YX, Bc, dV, bc, hm, LC, aW, bNq, az, mQ, aww, zC, bw, dc, ki, cj, Zn, m, aNn, dD, n, aj, BF, uE, JX, cU, h, ey, Og, CQ, Ca, YF, adz, hq, ar, dC, eD, fL, B, Dw, fM, lk, jx, v, j, C, pd, q, J, mP, Cc, WY, jZ, rS, aT, cy, le, r, lo, t, Cv, zJ, baw, ig, hc, cht, wR, EY, fD, zA, bTc, aDj, azW, jh, eq, atK, mC, hX, yt, ds, LA, ji, NM, cH, Of, eZ, dN, aoF, gE, hp, uF, iV, aDH, bE, gZ, ei, y); #c1(MYD88) c2(NP_001166039) c3(myeloid differentiation primary response protein MyD88 isoform 3) c4(68110) c5(68111, 68112, 68113, 68114, 68115, 68116) c6( ) c7(oO, bM, T, fR, ea, fd, bB, ci, xE, vV, YX, Bc, dV, bc, hm, LC, aW, bNq, az, ml, aw, zC, bw, dc, ki, cj, Zn, m, aNn, dD, n, aj, BF, uE, JX, cU, h, ey, Og, CQ, Ca, YF, adz, hq, ar, dC, eD, fL, B, Dw, fM, lk, jx, v, j, C, pd, q, J, mP, Cc, WY, jZ, rS, aT, cy, le, r, lo, t, Cv, zJ, baw, ig, hc, cht, wR, EY, fD, zA, bTc, aDj, azW, jh, eq, atK, mC, hX, yt, ds, LA, ji, NM, cH, Of, eZ, dN, aoF, gE, hp, uF, iV, aDH, bE, gZ, ei, y); #c1(MYD88) c2(NP_001166040) c3(myeloid differentiation primary response protein MyD88 isoform 4) c4(68117) c5(68118, 68119, 68120, 68121, 68122, 68123, 68124, 68125, 68126, 68127, 68128) c6( ) c7(oO, bM, T, fR, ea, fd, bB, ci, xE, vV, YX, Bc, dV, bc, hm, LC, aW, bNq, az, ml, awv, zC, bw, dc, ki, cj, Zn, m, aNn, dD, n, aj, BF, uE, JX, cU, h, ey, Og, CO, Ca, YF, adz, hq, ar, dC, eD, fL, B, Dw, fM, lk, jx, v, j, C, pd, q, J, mP, Cc, WY, jZ, rS, aT, cy, le, r, lo, t, Cv, zJ, baw, ig, hc, cht, wR, EY, fD, zA, bTc, aDj, azW, jh, eq, atK, mC, hX, yt, ds, LA, ji, NM, cH, Of, eZ, dN, aoF, gE, hp, uF, iV, aDH, bE, gZ, ei, y); #c1(MYD88) c2(NP_002459) c3(myeloid differentiation primary response protein MyD88 isoform 2) c4(129016, 129017, 129018) c5(129019, 129020, 129021, 129022, 129023, 129024, 129025, 129026, 129027, 129028, 129029, 129030, 129031) c6( ) c7(oO, bM, T, fR, ea, fd, bB, ci, xE, vV, YX, Bc, dV, bc, hm, LC, aW, bNq, az, md, aw, zC, bw, dc, ki, cj, Zn, m, aNn, dD, n, aj, BF, uE, JX, cU, h, ey, Og, CO, Ca, YF, adz, hq, ar, dC, eD, fL, B, Dw, fM, lk, jx, v, j, C, pd, q, J, mP, Cc, WY, jZ, rS, aT, cy, le, r, lo, t, Cv, zJ, baw, ig, hc, cht, wR, EY, fD, zA, bTc, aDj, azW, jh, eq, atK, mC, hX, yt, ds, LA, ji, NM, cH, Of, eZ, dN, aoF, gE, hp, uF, iV, aDH, bE, gZ, ei, y); #c1(MYDGF) c2(NP_061980) c3(UPF0556 protein C19orf10 precursor) c4(201602) c5(201603, 201604, 201605, 201606, 201607, 201608) c6( ) c7(oO, bM, cZ, T, dD, JZ, ea, ht, al, jh, fF, aj, v, j, jE, C, eq, aoj, V, qW, ai, J, ad, aDF, hX, rw, ds, Os, aT, bQd, K, ji, dd, hu, ae, DA, Nr, A, cW, aW, Y, oV, fO, cr, fe, po, lo, iV, eE, axp, bz, DF, Nq, ck, aDu, W, eM, ig, aCT, bE, z, at, dW, eo, m, fD, y, bt, Pr, B); #c1(MYEF2) c2(NP_001288139) c3(myelin expression factor 2 isoform b) c4(113601, 113602, 113603) c5(113604, 113605, 113606, 113607, 113608, 113609, 113610) c6( ) c7(aw, anR); #c1(MYEF2) c2(NP_057216) c3(myelin expression factor 2 isoform a) c4(189530) c5(189531, 189532, 189533, 189534, 189535, 189536) c6( ) c7(aw, anR); #c1(MYEF2) c2(XP_005254481) c3(myelin expression factor 2 isoform X2) c4( ) c5(273226, 273227, 273228, 273229, 273230) c6( ) c7(aw, anR); #c1(MYEF2) c2(XP_011519959) c3(myelin expression factor 2 isoform X5) c4( ) c5(314780, 314781, 314782, 314783, 314784, 314785) c6( ) c7(aw, anR); #c1(MYEOV) c2(NP_001280220) c3(myeloma-overexpressed gene protein isoform 1) c4(111485) c5(111486, 111487, 111488, 111489, 111490, 111491, 111492, 111493, 111494, 111495) c6( ) c7(T, Jl, aW, bE, ff, r, cj, aT, bB, fd, en, g, cm, it, q); #c1(MYF5) c2(NP_005584) c3(myogenic factor 5) c4(157691) c5(157692, 157693, 157694, 157695, 157696, 157697, 157698) c6( ) c7(bv, ac, Qr, v, qk, cd); #c1(MYF6) c2(NP_002460) c3(myogenic factor 6) c4(129032) c5(129033, 129034, 129035, 129036, 129037, 129038, 129039) c6( ) c7(bv, chu, oj, uY, Xu, Wq, bhe); #c1(MYH1) c2(NP_005954) c3(myosin-1) c4(160693, 160694) c5(160695, 160696, 160697, 160698, 160699, 160700, 160701, 160702, 160703, 160704, 160705, 160706) c6( ) c7(bF, XW, jz, lt, aW, nX, aA, r, aT, hN, aq, aF, g, rk); #c1(MYH10) c2(NP_001242941) c3(myosin-10 isoform 1) c4(89330) c5(89331, 89332, 89333, 89334, 89335) c6( ) c7(cF, xu, aW, aT, vw, bt); #c1(MYH10) c2(NP_005955) c3(myosin-10 isoform 2) c4(160707, 160708) c5(160709, 160710, 160711, 160712, 160713, 160714, 160715, 160716, 160717, 160718) c6( ) c7(cF, xu, aW, aT, vw, bt); #c1(MYH10) c2(XP_011522177) c3(myosin-10 isoform X2) c4( ) c5(316775) c6( ) c7(cF, xu, aW, aT, vw, bt); #c1(MYH10) c2(XP_011522179) c3(myosin-10 isoform X4) c4(316776) c5(316777, 316778, 316779, 316780, 316781, 316782) c6( ) c7(cF, xu, aW, aT, vw, bt); #c1(MYH10) c2(XP_011522180) c3(myosin-10 isoform X5) c4(316783) c5(316784, 316785, 316786, 316787, 316788, 316789) c6( ) c7(cF, xu, aW, aT, vw, bt); #c1(MYH10) c2(XP_011522182) c3(myosin-10 isoform X7) c4( ) c5(316790, 316791) c6( ) c7(cF, xu, aW, aT, vw, bt); #c1(MYH11) c2(NP_001035202) c3(myosin-11 isoform SM2B) c4(32598, 32599) c5(32600, 32601, 32602, 32603, 32604, 32605, 32606, 32607, 32608, 32609, 32610) c6( ) c7(chv, bM, T, Ap, fo, hF, q, ad, fX, chw, gd, aT, iY, K, j, chx, ae, aBl, Nr, bf, BO, s, iU, aW, r, po, Au, Fu, bw, yR, Nq, ck, iT, be, MC, aRD, bt, fl); #c1(MYH11) c2(NP_001035203) c3(myosin-11 isoform SM1B) c4(32611) c5(32612, 32613, 32614, 32615, 32616, 32617, 32618) c6( ) c7(chv, bM, T, Ap, fo, hF, q, ad, fX, chw, gd, aT, iY, K, j, chx, ae, aBl, Nr, bf, BO, s, iU, aW, r, po, Au, Fu, bw, yR, Nq, ck, iT, be, MC, aRD, bt, fl); #c1(MYH11) c2(NP_002465) c3(myosin-11 isoform SM1A) c4(129102) c5(129103, 129104, 129105, 129106, 129107, 129108, 129109) c6( ) c7(chv, bM, T, Ap, fo, hF, q, ad, fx, chw, gd, aT, iY, K, j, chx, ae, aBl, Nr, bf, BO, s, iU, aW, r, po, Au, Fu, bw, yR, Nq, ck, iT, be, MC, aRD, bt, fl); #c1(MYH11) c2(NP_074035) c3(myosin-11 isoform SM2A) c4(213491) c5(213492, 213493, 213494, 213495, 213496) c6( ) c7(chv, bM, T, Ap, fo, hF, q, ad, fX, chw, gd, aT, iY, K, j, chx, ae, aBl, Nr, bf, BO, s, iU, aW, r, po, Au, Fu, bw, yR, Nq, ck, iT, be, MC, aRD, bt, fl); #c1(MYH13) c2(NP_003793) c3(myosin-13) c4(140894, 140895) c5(140896, 140897, 140898, 140899, 140900, 140901, 140902, 140903, 140904, 140905, 140906, 140907, 140908, 140909, 140910, 140911) c6( ) c7(af); #c1(MYH14) c2(NP_001139281) c3(myosin-14 isoform 3) c4(59087, 59088, 59089) c5(59090, 59091, 59092, 59093, 59094, 59095, 59096, 59097, 59098, 59099, 59100, 59101, 59102, 59103, 59104, 59105, 59106, 59107, 59108, 59109, 59110, 59111) c6( ) c7(chz, Na, akf, T, cc, eR, chA, mx, jh, vc, chy, aS, Nc, bX, Tp, Bh, ad); #c1(MYH14) c2(NP_079005) c3(myosin-14 isoform 2) c4(216530) c5( ) c6( ) c7(chz, Na, akf, T, cc, eR, chA, mx, jh, vc, chy, aS, Nc, bX, Tp, Bh, ad); #c1(MYH14) c2(XP_011525622) c3(myosin-14 isoform X2) c4( ) c5(320028) c6( ) c7(chz, Na, akf, T, cc, eR, chA, mx, jh, vc, chy, aS, Nc, bX, Tp, Bh, ad); #c1(MYH14) c2(XP_011525623) c3(myosin-14 isoform X3) c4(320029) c5(320030, 320031, 320032, 320033, 320034, 320035, 320036, 320037, 320038, 320039, 320040, 320041) c6( ) c7(chz, Na, akf, T, cc, eR, chA, mx, jh, vc, chy, aS, Nc, bX, Tp, Bh, ad); #c1(MYH14) c2(XP_011525625) c3(myosin-14 isoform X1) c4(320042) c5(320043, 320044, 320045, 320046, 320047) c6( ) c7(chz, Na, akf, T, cc, eR, chA, mx, jh, vc, chy, aS, Nc, bX, Tp, Bh, ad); #c1(MYH15) c2(NP_055796) c3(myosin-15 precursor) c4(183563) c5(183564, 183565, 183566, 183567, 183568, 183569) c6( ) c7(dD, ac, ea, e, aw, j, eq, aHP, aHQ, eu); #c1(MYH2) c2(NP_001093582) c3(myosin-2) c4(40279, 40280, 40281, 40282) c5(40283, 40284, 40285, 40286, 40287, 40288, 40289, 40290, 40291, 40292, 40293, 40294, 40295, 40296, 40297, 40298, 40299, 40300, 40301, 40302, 40303, 40304, 40305, 40306, 40307, 40308, 40309, 40310) c6( ) c7(ul, aZA, buA, eA, chC, XW, cc, Ae, oj, Ak, By, vc, ji, K, chD, yi, Y, XV, Ad, aaO, chB, cl, Ew, cj, bt, AI); #c1(MYH3) c2(XP_011522173) c3(myosin-3 isoform X1) c4(316762) c5(316763, 316764, 316765, 316766, 316767, 316768, 316769, 316770, 316771, 316772) c6( ) c7(chE, XW, chF, ff, ht, oj, dV, buA, bBU, K, en, chG); #c1(MYH3) c2(XP_011522174) c3(myosin-3 isoform X2) c4( ) c5(316773, 316774) c6( ) c7(chE, XW, chF, ff, ht, oj, dV, buA, bBU, K, en, chG); #c1(MYH4) c2(NP_060003) c3(myosin-4) c4(193607) c5(193608, 193609, 193610, 193611, 193612, 193613) c6( ) c7(oe, My); #c1(MYH6) c2(NP_002462) c3(myosin-6) c4(129040) c5(129041, 129042, 129043, 129044, 129045, 129046, 129047, 129048, 129049, 129050) c6( ) c7(T, AY, chH, wV, j, cd, ad, chK, cc, fm, chI, oj, vc, hn, bHm, rz, ae, alj, mG, mx, bD, chJ); #c1(MYH7) c2(NP_000248) c3(myosin-7) c4(5731, 5732) c5(5733, 5734, 5735, 5736, 5737, 5738, 5739, 5740, 5741, 5742, 5743, 5744) c6( ) c7(chM, AY, bAB, mr, acl, aw, cz, chL, n, aNG, cd, boz, NW, cc, Ws, boB, oj, kf, yu, vc, atw, aWs, chN, eN, mu, aTm, yi, anR, Ad, akB, chy, app, mo, aYo, bji, bfW, Bt, cl, mx, uS, bD, gq, vQ); #c1(MYH7B) c2(NP_065935) c3(myosin-7B) c4(206322, 206323) c5(206324, 206325, 206326, 206327, 206328, 206329, 206330) c6( ) c7(chz, cc, Dy, hk, Y, aT, dL, bX); #c1(MYH7B) c2(XP_011527244) c3(myosin-7B isoform X2) c4( ) c5(321823, 321824, 321825, 321826, 321827) c6( ) c7(chz, cc, Dy, hk, Y, aT, dL, bX); #c1(MYH7B) c2(XP_011527251) c3(myosin-7B isoform X8) c4(321828, 321829, 321830, 321831) c5(321832, 321833, 321834, 321835, 321836, 321837, 321838, 321839, 321840, 321841, 321842, 321843, 321844, 321845, 321846, 321847, 321848, 321849, 321850, 321851, 321852, 321853, 321854, 321855) c6( ) c7(chz, cc, Dy, hk, Y, aT, dL, bX); #c1(MYH7B) c2(XP_011527252) c3(myosin-7B isoform X9) c4( ) c5(321856) c6( ) c7(chz, cc, Dy, hk, Y, aT, dL, bX); #c1(MYH8) c2(NP_002463) c3(myosin-8) c4(129051) c5(129052, 129053, 129054, 129055, 129056, 129057, 129058, 129059, 129060, 129061, 129062, 129063, 129064, 129065, 129066) c6( ) c7(bBU, chO, chG, XW, chP); #c1(MYH9) c2(NP_002464) c3(myosin-9) c4(129067, 129068, 129069, 129070, 129071) c5(129072, 129073, 129074, 129075, 129076, 129077, 129078, 129079, 129080, 129081, 129082, 129083, 129084, 129085, 129086, 129087, 129088, 129089, 129090, 129091, 129092, 129093, 129094, 129095, 129096, 129097, 129098, 129099, (29100, 129101) c6( ) c7(bL, eq, T, dD, chR, aZA, chV, xu, qb, ea, eR, uv, chU, aA, cz, che, aj, ah, HS, uE, Wa, ml, aY, uW, ye, hF, J, eW, aXI, chC, fU, Na, amJ, bpp, aT, vw, h, chX, Nc, chW, DU, ey, DY, sj, UM, Nr, acF, aW, ajP, eZ, Xm, aF, bz, bLL, Nq, sk, pa, apM, BU, ac, Pa, chA, hc, mG, MM, dy, od, bt, chT, chS); #c1(MYL1) c2(NP_524144) c3(myosin light chain 1/3, skeletal muscle isoform isoform ff) c4(231132) c5(231133, 231134, 231135, 231136, 231137, 231138) c6( ) c7(bv); #c1(MYL1) c2(NP_524146) c3(myosin light chain 1/3, skeletal muscle isoform isoform 3f) c4(231139) c5(231140, 231141, 231142, 231143, 231144, 231145) c6( ) c7(bv); #c1(MYL12A) c2(NP_001289977) c3(myosin regulatory light chain (2A isoform 1) c4( ) c5(114894, 114895, 114896, 114897, 114898, 114899, 114900, 114901, 114902, 114903) c6( ) c7(vc, bz, cl, cc, py); #c1(MYL12A) c2(NP_001289978) c3(myosin regulatory light chain (ZA isoform 2) c4(114904) c5( ) c6( ) c7(vc, bz, cl, cc, py); #c1(MYL12B) c2(NP_001138416) c3(myosin regulatory light chain (28) c4(56623) c5(56624, 56625, 56626, 56627, 56628, 56629, 56630, 56631, 56632, 56633, 56634, 56635, 56636, 56637, 56638, 56639, 56640, 56641, 56642, 56643, 56644) c6( ) c7(aj, chY); #c1(MYL2) c2(NP_000423) c3(myosin regulatory light chain 2, ventricular/cardiac muscle isoform) c4(8810) c5(8811, 8812, 8813, 8814, 8815, 8816) c6( ) c7(bL, cc, chZ, mx, cz, vc, fr, K, mo, eA, cfe); #c1(MYL3) c2(NP_000249) c3(myosin light chain 3) c4(5745, 5746) c5(5747, 5748, 5749, 5750, 5751, 5752, 5753, 5754, 5755, 5756, 5757, 5758, 5759, 5760) c6( ) c7(yu, vc, cc, mx, cia); #c1(MYL4) c2(NP_001002841) c3(myosin light chain 4) c4(19624, 19625) c5(19626, 19627, 19628, 19629, 19630, 19631, 19632, 19633, 19634, 19635, 19636, 19637, 19638, 19639, 19640, 19641) c6( ) c7(bkZ, aPn, bwE, aNN, vc, bK, mk, as, cb, dr); #c1(MYL6) c2(NP_066299) c3(myosin light polypeptide 6 isoform 1) c4(207130) c5(207131, 207132, 207133, 207134, 207135, 207136, 207137, 207138, 207139, 207140) c6( ) c7(K); #c1(MYL6) c2(NP_524147) c3(myosin light polypeptide 6 isoform 2) c4(231146) c5(231147, 231148, 231149, 231150, 231151, 231152) c6( ) c7(K); #c1(MYL7) c2(NP_067046) c3(myosin regulatory light chain 2, atrial isoform) c4(208699, 208700) c5(208701, 208702, 208703, 208704, 208705, 208706, 208707, 208708, 208709, 208710, 208711, 208712) c6( ) c7(cc, aPn, vc, j, K, cb, f, cd, vQ); #c1(MYL9) c2(NP_006088) c3(myosin regulatory light polypeptide 9 isoform a) c4(161809) c5(161810, 161811, 161812, 161813, 161814, 161815, 161816) c6( ) c7(cc, cl, vc, fr, aS, K, mk, bt); #c1(MYL9) c2(NP_852667) c3(myosin regulatory light polypeptide 9 isoform b) c4(255111) c5(255112, 255113, 255114, 255115, 255116, 255117) c6( ) c7(cc, cl, vc, fr, aS, K, mk, bt); #c1(MYL1P) c2(NP_037394) c3(E3 ubiquitin-protein ligase MYL1P) c4(175234, 175235) c5(175236, 175237, 175238, 175239, 175240, 175241, 175242, 175243) c6( ) c7(bL, dP, T, x, aE, c, bE, cj, kH, bB, v, j, k, eq, fr, iK, B); #c1(MYL1P) c2(XP_005249090) c3(E3 ubiquitin-protein ligase MYL1P isoform X2) c4( ) c5(268980, 268981, 268982, 268983, 268984) c6( ) c7(bL, dP, T, x, aE, c, bE, cj, kH, bB, v, j, k, eq, fr, iK, B); #c1(MYLK) c2(NP_444253) c3(myosin light chain kinase, smooth muscle isoform 1) c4(230118) c5(230119, 230120, 230121, 230122, 230123, 230124, 230125, 230126, 230127, 230128) c6( ) c7(bM, T, Lx, cz, Bm, n, BH, fF, j, C, cd, ad, aJr, eA, eW, no, yt, sl, aT, jy, K, ae, aa, cH, cib, aW, md, fe, dw, Bu, bz, qa, dR, es, D, eM, iA, bm, sG, mf, aS, mk, fD, bD); #c1(MYLK) c2(NP_444254) c3(myosin light chain kinase, smooth muscle isoform 2) c4(230129, 230130, 230131) c5(230132, 230133, 230134, 230135, 230136, 230137, 230138, 230139, 230140, 230141, 230142, 230143) c6( ) c7(bM, T, Lx, cz, Bm, n, BH, fF, j, C, cd, ad, aJr, eA, eW, no, yt, sl, aT, jy, K, ae, aa, cH, cib, aW, mQ, fe, dw, Bu, bz, qa, dR, es, D, eM, iA, bm, sG, mf, aS, mk, fD, bD); #c1(MYLK) c2(NP_444255) c3(myosin light chain kinase, smooth muscle isoform 3A) c4(230144) c5(230145, 230146, 230147, 230148, 230149, 230150, 230151, 230152, 230153, 230154) c6( ) c7(bM, T, Lx, cz, Bm, n, BH, fF, j, C, cd, ad, aJr, eA, eW, no, yt, sl, aT, jy, K, ae, aa, cH, cib, aW, mQ, fe, dw, Bu, bz, qa, dR, es, D, eM, iA, bm, sG, mf, aS, mk, fD, bD); #c1(MYLK) c2(NP_444256) c3(myosin light chain kinase, smooth muscle isoform 3B) c4( ) c5(230155, 230156, 230157, 230158, 230159, 230160) c6( ) c7(bM, T, Lx, cz, Bm, n, BH, fF, j, C, cd, ad, aJr, eA, eW, no, yt, sl, aT, jy, K, ae, aa, cH, cib, aW, mQ, fe, dw, Bu, bz, qa, dR, es, D, eM, iA, bm, sG, mf, aS, mk, fD, bD); #c1(MYLK) c2(NP_444260) c3(myosin light chain kinase, smooth muscle isoform 8) c4(230161) c5(230162, 230163, 230164, 230165, 230166, 230167) c6( ) c7(bM, T, Lx, cz, Bm, n, BH, fF, j, C, cd, ad, aJr, eA, eW, no, yt, sl, aT, jy, K, ae, aa, cH, cib, aW, md, fe, dw, Bu, bz, qa, dR, es, D, eM, iA, bm, sG, mf, aS, mk, fD, bD); #c1(MYLK2) c2(NP_149109) c3(myosin light chain kinase 2, skeletal/cardiac muscle) c4(227192) c5(227193, 227194, 227195, 227196, 227197, 227198, 227199, 227200, 227201, 227202) c6( ) c7(yt, Bu, AY, no, cc); #c1(MYLK3) c2(NP_872299) c3(myosin light chain kinase 3) c4(256044, 256045) c5(256046, 256047, 256048, 256049, 256050, 256051, 256052) c6( ) c7(Bu, no); #c1(MYLPF) c2(NP_037424) c3(myosin regulatory light chain 2, skeletal muscle isoform) c4(175488) c5(175489, 175490, 175491, 175492, 175493, 175494) c6( ) c7(eA, j, cfe); #c1(MYNN) c2(NP_001172048) c3(myoneurin isoform B) c4(72355) c5(72356, 72357, 72358, 72359, 72360, 72361, 72362) c6( ) c7(at, V, dL, hp); #c1(MYNN) c2(XP_011511290) c3 (myoneurin isoform X1) c4(301781, 301782) c5(301783, 301784, 301785, 301786, 301787, 301788, 301789, 301790, 301791, 301792, 301793, 301794, 301795, 301796, 301797, 301798, 301799) c6( ) c7(at, V, dL, hp); #c1(MY010) c2(NP_036466) c3(unconventional myosin-X) c4(174055) c5(174056, 174057, 174058, 174059, 174060, 174061, 174062, 174063, 174064, 174065, 174066, 174067, 174068, 174069, 174070, 174071, 174072, 174073, 174074, 174075, 174076, 174077, 174078, 174079, 174080) c6( ) c7(aW, om, cz, af, aT); #c1(MY010) c2(XP_005248363) c3(unconventional myosin-X isoform X2) c4(268370) c5( ) c6( ) c7(aW, om, cz, af, aT); #c1(MY010) c2(XP_005248364) c3(unconventional myosin-X isoform X3) c4(268371) c5( ) c6( ) c7(aW, om, cz, af, al); #c1(MY015A) c2(NP_057323) c3(unconventional myosin-XV) c4(190114, 190115, 190116, 190117, 190118) c5(190119, 190120, 190121, 190122, 190123, 190124, 190125, 190126, 190127, 190128, 190129, 190130, 190131, 190132, 190133, 190134, 190135, 190136, 190137, 190138, 190139, 190140, 190141) c6( ) c7(bCd, cic, alA, aQi, bU, Bh, bCJ); #c1(MY015A) c2(XP_011522223) c3(unconventional myosin-XV isoform X5) c4( ) c5(316843, 316844, 316845, 316846, 316847, 316848, 316849) c6( ) c7(bCd, cic, alA, aQi, bU, Bh, bCJ); #c1(MY016) c2(NP_001185879) c3(unconventional myosin-XVI isoform 1) c4( ) c5(76625, 76626, 76627, 76628) c6( ) c7(W, ac, aj, dy, j, rz, cd, J); #c1(MY016) c2(NP_055826) c3(unconventional myosin-XVI isoform 2) c4(183724, 183725, 183726, 183727, 183728) c5(183729, 183730, 183731, 183732, 183733, 183734, 183735, 183736, 183737, 183738, 183739, 183740, 183741, 183742, 183743, 183744, 183745, 183746, 183747, 183748, 183749, 183750, 183751, 183752, 183753, 183754, 183755, 183756, 183757, 183758) c6( ) c7(W, ac, aj, dy, j, rz, cd, J); #c1(MY018B) c2(NP_115997) c3(unconventional myosin-XVIIIb) c4(225172) c5(225173, 225174, 225175, 225176, 225177, 225178, 225179, 225180, 225181, 225182) c6( ) c7(av, ft, ib, W, r, he, m, t, bz, q); #c1(MYD1A) c2(NP_005370) c3(unconventional myosin-1a) c4(155505, 155506, 155507) c5(155508, 155509, 155510, 155511, 155512, 155513, 155514, 155515, 155516, 155517, 155518, 155519, 155520, 155521, 155522, 155523) c6( ) c7(nO, mG, cid, aA, aq, aF, B, Qe); #c1(MYO1A) c2(XP_011536675) c3 (unconventional myosin-1a isoform X1) c4( ) c5(334183, 334184, 334185, 334186, 334187, 334188) c6( ) c7(nO, mG, cid, aA, aq, aF, B, Qe); #c1(MY01B) c2(NP_036355) c3(unconventional myosin-1b isoform 2) c4(173164, 173165) c5(173166, 173167, 173168, 173169, 173170, 173171, 173172, 173173, 173174, 173175, 173176, 173177, 173178, 173179, 173180, 173181, 173182, 173183, 173184, 173185, 173186, 173187, 173188, 173189, 173190, 173191, 173192, 173193, 173194, 173195, 173196) c6( ) c7(fq, A); #c1(MY01B) c2(XP_011509515) c3(unconventional myosin-1b isoform X1) c4(300086, 300087, 300088, 300089) c5(300090, 300091, 300092, 300093, 300094, 300095, 300096, 300097, 300098, 300099, 300100, 300101, 300102, 300103, 300104) c6( ) c7(fq, A); #c1(MYD1B) c2(XP_011509516) c3 (unconventional myosin-1b isoform X2) c4(300105, 300106) c5(300107, 300108, 300109, 300110, 300111, 300112, 300113, 300114, 300115, 300116, 300117, 300118) c6( ) c7(fq, A); #c1(MYD1B) c2(XP_011509517) c3(unconventional myosin-1b isoform X3) c4(300119) c5( ) c6( ) c7(fq, A); #c1(MYO1C) c2(NP_001074248) c3(unconventional myosin-1c isoform a) c4(37049, 37050) c5(37051, 37052, 37053, 37054, 37055, 37056, 37057, 37058, 37059, 37060, 37061, 37062, 37063, 37064, 37065, 37066, 37067, 37068, 37069, 37070, 37071, 37072, 37073, 37074) c6( ) c7(T, mG, n, dL, m, t, eL); #c1(MYO1C) c2(NP_001074419) c3(unconventional myosin-1c isoform b) c4(37097, 37098, 37099, 37100) c5(37101, 37102, 37103, 37104, 37105, 37106, 37107, 37108, 37109, 37110, 37111, 37112) c6( ) c7(T, mG, n, dL, m, t, eL); #c1(MYO1D) c2(NP_056009) c3 (unconventional myosin-Id isoform 1) c4(185105, 185106) c5(185107, 185108, 185109, 185110, 185111, 185112, 185113, 185114, 185115, 185116, 185117, 185118) c6( ) c7(at, aC, bN, cz); #c1(MYO1E) c2(NP_004989) c3(unconventional myosin-1e) c4(151772) c5(151773, 151774, 151775, 151776, 151777, 151778) c6( ) c7(oe, gi, mG, vw, ah, cie); #c1(MYO1F) c2(NP_036467) c3(unconventional myosin-1f) c4(174081, 174082) c5(174083, 174084, 174085, 174086, 174087, 174088, 174089, 174090, 174091, 174092, 174093, 174094) c6( ) c7(mG, ey, iZ); #c1(MYO1F) c2(XP_011526330) c3(unconventional myosin-1f isoform X5) c4( ) c5(320633, 320634) c6( ) c7(mG, ey, iZ); #c1(MY03A) c2(XP_011517803) c3(myosin-IIIa isoform X1) c4(311725, 311726) c5(311727, 311728, 311729, 311730, 311731, 311732) c6( ) c7(cif, r, bX, Z, y, q); #c1(MY03A) c2(XP_011517806) c3(myosin-IIIa isoform X4) c4(311733) c5( ) c6( ) c7(cif, r, bX, Z, y, q); #c1(MY03A) c2(XP_011517807) c3(myosin-IIIa isoform X5) c4(311734) c5( ) c6( ) c7(cif, r, bX, Z, y, q); #c1(MY03A) c2(XP_011517813) c3(myosin-IIIa isoform X11) c4( ) c5(311735, 311736, 311737, 311738, 311739, 311740, 311741, 311742, 311743, 311744, 311745, 311746, 311747, 311748, 311749, 311750, 311751, 311752) c6( ) c7(cif, r, bX, Z, y, q); #c1(MY03B) c2(NP_001077084) c3(myosin-IIIb isoform 1) c4(37708) c5(37709, 37710, 37711, 37712, 37713, 37714) c6( ) c7(Tp); #c1(MY03B) c2(NP_620482) c3(myosin-IIIb isoform 2) c4(237701) c5(237702, 237703, 237704, 237705, 237706, 237707, 237708) c6( ) c7(Tp); #c1(MY03B) c2(XP_006712362) c3(myosin-IIIb isoform X2) c4(286603, 286604) c5(286605, 286606, 286607, 286608, 286609, 286610, 286611, 286612, 286613, 286614, 286615) c6( ) c7(Tp); #c1(MY05A) c2(NP_000250) c3(unconventional myosin-Va isoform 1) c4(5761) c5(5762, 5763, 5764, 5765, 5766, 5767, 5768, 5769, 5770, 5771, 5772, 5773) c6( ) c7(cig, ceE, T, hk, Y, cih, gd, at, dL, ey, bX); #c1(MY05A) c2(NP_001135967) c3(unconventional myosin-Va isoform 2) c4(53747, 53748) c5(53749, 53750, 53751, 53752, 53753, 53754, 53755, 53756, 53757, 53758, 53759, 53760, 53761, 53762, 53763, 53764, 53765, 53766) c6( ) c7(cig, ceE, T, hk, Y, cih, gd, at, dL, ey, bX); #c1(MY05A) c2(XP_005254454) c3 (unconventional myosin-Va isoform X3) c4(273219) c5(273220, 273221, 273222, 273223, 273224, 273225) c6( ) c7(cig, ceE, T, hk, Y, cih, gd, at, dL, ey, bX); #c1(MY05A) c2(XP_011519911) c3(unconventional myosin-Va isoform X6) c4(314697) c5(314698, 314699, 314700, 314701, 314702, 314703) c6( ) c7(cig, ceE, T, hk, Y, cih, gd, at, dL, ey, bX); #c1(MY05A) c2(XP_011519912) c3(unconventional myosin-Va isoform X7) c4(314704) c5( ) c6( ) c7(cig, ceE, T, hk, Y, cih, gd, at, dL, ey, bX); #c1(MY05A) c2(XP_011519913) c3(unconventional myosin-Va isoform X8) c4(314705) c5( ) c6( ) c7(cig, ceE, T, hk, Y, cih, gd, at, dL, ey, bX); #c1(MY05B) c2(NP_001073936) c3(unconventional myosin-Vb) c4(36596, 36597, 36598) c5(36599, 36600, 36601, 36602, 36603, 36604, 36605, 36606, 36607, 36608, 36609, 36610, 36611, 36612, 36613, 36614, 36615, 36616) c6( ) c7(mO, cii, cA, bpj, lz, ju, hl, awd, aA, n, aj, aq, Km, bK, aF, oc, hJ, xa); #c1(MY05C) c2(NP_061198) c3(unconventional myosin-Vc) c4(199924, 199925) c5(199926, 199927, 199928, 199929, 199930, 199931, 199932, 199933, 199934, 199935, 199936, 199937, 199938, 199939) c6( ) c7(IB); #c1(MY05C) c2(XP_011520083) c3(unconventional myosin-Vc isoform X1) c4(314979, 314980, 314981) c5(314982, 314983, 314984, 314985, 314986, 314987, 314988, 314989, 314990, 314991, 314992, 314993, 314994, 314995, 314996, 314997) c6( ) c7(B); #c1(MY06) c2(NP_001287828) c3(unconventional myosin-VI isoform 2) c4(112943) c5( ) c6( ) c7(cik, ae, T, mG, cz, aQV, cij, cil, hn, ci, lB, Bh, ad); #c1(MY06) c2(NP_004990) c3(unconventional myosin-VI isoform 1) c4(151779) c5(151780, 151781, 151782, 151783, 151784, 151785, 151786, 151787, 151788, 151789, 151790, 151791, 151792, 151793, 151794, 151795, 151796, 151797, 151798, 151799, 151800, 151801, 151802, 151803, 151804, 151805, 151806, 151807, 151808, 151809) c6( ) c7(cik, ae, T, mG, cz, aQV, cij, cil, hn, ci, lB, Bh, ad); #c1(MY06) c2(XP_005248776) c3(unconventional myosin-VI isoform X1) c4(268574) c5( ) c6( ) c7(cik, ae, T, mG, cz, aQV, cij, cil, hn, ci, lB, Bh, ad); #c1(MY06) c2(XP_005248777) c3 (unconventional myosin-VI isoform X2) c4(268575) c5( ) c6( ) c7(cik, ae, T, mG, cz, aQV, cij, cil, hn, ci, lB, Bh, ad); #c1(MY06) c2(XP_005248778) c3 (unconventional myosin-VI isoform X3) c4(268576) c5(268577, 268578, 268579, 268580, 268581, 268582) c6( ) c7(cik, ae, T, mG, cz, aQV, cij, cil, hn, ci, lB, Bh, ad); #c1(MY06) c2(XP_005248783) c3 (unconventional myosin-VI isoform X6) c4(268583) c5( ) c6( ) c7(cik, ae, T, mG, cz, aQV, cij, cil, hn, ci, lB, Bh, ad); #c1(MY07A) c2(NP_000251) c3(unconventional myosin-VIIa isoform 1) c4(5774, 5775, 5776) c5(5777, 5778, 5779, 5780, 5781, 5782, 5783, 5784, 5785, 5786, 5787, 5788, 5789, 5790, 5791, 5792, 5793, 5794) c6( ) c7(dD, nx, n, eq, oc, Nh, Fs, alx, cim, IR, Y, cds, Xz, Tp, nC, cio, cin, W, hk, mG, nw, Bh); #c1(MY07A) c2(NP_001120651) c3(unconventional myosin-VIIa isoform 3) c4(44374, 44375) c5(44376, 44377, 44378, 44379, 44380, 44381, 44382, 44383, 44384, 44385, 44386, 44387) c6( ) c7(dD, nx, n, eq, oc, Nh, Fs, alx, cim, lR, Y, cds, Xz, Tp, nC, cio, cin, W, hk, mG, nw, Bh); #c1(MY07A) c2(NP_001120652) c3 (unconventional myosin-VIIa isoform 2) c4(44388) c5(44389, 44390, 44391, 44392, 44393, 44394) c6( ) c7(dD, nx, n, eq, oc, Nh, Fs, alx, cim, lR, Y, cds, Xz, Tp, nC, cio, cin, W, hk, mG, nw, Bh); #c1(MY07A) c2(XP_006718624) c3(unconventional myosin-VIIa isoform X14) c4(291942) c5(291943, 291944, 291945, 291946, 291947, 291948) c6( ) c7(dD, nx, n, eq, oc, Nh, Fs, alx, cim, lR, Y, cds, Xz, Tp, nC, cio, cin, W, hk, mG, nw, Bh); #c1(MY07A) c2(XP_011543351) c3(unconventional myosin-VIIa isoform X10) c4(342132) c5(342133, 342134, 342135, 342136, 342137, 342138) c6( ) c7(dD, nx, n, eq, oc, Nh, Fs, alx, cim, lR, Y, cds, Xz, Tp, nC, cio, cin, W, hk, mG, nw, Bh); #c1(MY07A) c2(XP_011543352) c3(unconventional myosin-VIIa isoform X11) c4(342139) c5(342140, 342141, 342142, 342143, 342144, 342145) c6( ) c7(dD, nx, n, eq, oc, Nh, Fs, alx, cim, lR, Y, cds, Xz, Tp, nC, cio, cin, W, hk, mG, nw, Bh); #c1(MY07A) c2(XP_011543354) c3(unconventional myosin-VIIa isoform X13) c4( ) c5(342146) c6( ) c7(dD, nx, n, eq, oc, Nh, Fs, alx, cim, lR, Y, cds, Xz, Tp, nC, cio, cin, W, hk, mG, nw, Bh); #c1(MY07B) c2(NP_001073996) c3(unconventional myosin-VIIb) c4(36854, 36855, 36856) c5(36857, 36858, 36859, 36860, 36861, 36862, 36863, 36864, 36865, 36866, 36867, 36868, 36869, 36870, 36871, 36872, 36873, 36874, 36875, 36876, 36877, 36878) c6( ) c7(dy, ac); #c1(MY09A) c2(NP_008832) c3 (unconventional myosin-IXa) c4(168911, 168912) c5(168913, 168914, 168915, 168916, 168917, 168918, 168919, 168920, 168921, 168922, 168923, 168924) c6( ) c7(Tp); #c1(MY09A) c2(XP_006720602) c3 (unconventional myosin-IXa isoform X4) c4(293580) c5(293581, 293582, 293583, 293584, 293585, 293586) c6( ) c7(Tp); #c1(MY09A) c2(XP_011519922) c3(unconventional myosin-IXa isoform X6) c4(314706) c5(314707, 314708, 314709, 314710, 314711, 314712) c6( ) c7(Tp); #c1(MY09A) c2(XP_011519923) c3 (unconventional myosin-(Xa isoform X7) c4(314713) c5( ) c6( ) c7(Tp); #c1(MY09A) c2(XP_011519924) c3 (unconventional myosin-IXa isoform X8) c4( ) c5(314714, 314715, 314716, 314717, 314718, 314719) c6( ) c7(Tp); #c1(MY09B) c2(NP_001123537) c3(unconventional myosin-IXb isoform 2) c4(47449) c5(47450, 47451, 47452, 47453, 47454, 47455, 47456, 47457) c6( ) c7(b, lt, cip, A, lV, eZ, fe, gy, hp, BF, wb, iV, i, f, V, y); #c1(MY09B) c2(NP_004136) c3 (unconventional myosin-IXb isoform 1) c4( ) c5(143840, 143841, 143842) c6( ) c7(b, lt, cip, A, lV, eZ, fe, gy, hp, BF, wb, iV, i, f, V, y); #c1(MY09B) c2(XP_011526332) c3(unconventional myosin-IXb isoform X2) c4(320635) c5( ) c6( ) c7(b, lt, cip, A, lV, eZ, fe, gy, hp, BF, wb, iV, i, f, V, y); #c1(MY09B) c2(XP_011526336) c3(unconventional myosin-IXb isoform X6) c4( ) c5(320636, 320637, 320638, 320639, 320640) c6( ) c7(b, lt, cip, A, lV, eZ, fe, gy, hp, BF, wb, iV, i, f, V, y); #c1(MYOC) c2(NP_000252) c3(myocilin precursor) c4(5795, 5796, 5797, 5798) c5(5799, 5800, 5801, 5802, 5803, 5804, 5805, 5806, 5807, 5808, 5809, 5810, 5811, 5812, 5813, 5814, 5815, 5816, 5817, 5818, 5819, 5820, 5821, 5822, 5823, 5824) c6( ) c7(HQ, cZ, DD, bbH, ec, vd, K, an, dd, bbG, lR, dH, wf, Pg, dw, bbE, vD, pZ, DW, ciq, Be, bbF, JJ, lJ, cir); #c1(MYOCD) c2(NP_001139784) c3(myocardin isoform 1) c4(59915, 59916) c5(59917, 59918, 59919, 59920, 59921, 59922) c6( ) c7(T, cc, VR, hW, aW, au, aT, Bm, cz, vc, Au, aS, K, aRD, hZ); #c1(MYOCD) c2(NP_705832) c3(myocardin isoform 2) c4(246296) c5(246297, 246298, 246299, 246300, 246301, 246302) c6( ) c7(T, cc, VR, hW, aW, au, aT, Bm, cz, vc, Au, aS, K, aRD, hZ); #c1(MYOCD) c2(XP_005256920) c3(myocardin isoform X1) c4(274582, 274583) c5(274584, 274585, 274586, 274587, 274588, 274589, 274590, 274591, 274592, 274593, 274594, 274595) c6( ) c7(T, cc, VR, hW, aW, au, aT, Bm, cz, vc, Au, aS, K, aRD, hZ); #c1(MYOCD) c2(XP_005256921) c3(myocardin isoform X2) c4( ) c5(274596, 274597, 274598, 274599, 274600, 274601) c6( ) c7(T, cc, VR, hW, aW, au, al, Bm, cz, vc, Au, aS, K, aRD, hZ); #c1(MYOD1) c2(NP_002469) c3(myoblast determination protein 1) c4(129110) c5(129111, 129112, 129113, 129114, 129115, 129116, 129117) c6( ) c7(T, FW, if, Or, eX, ml, iW, cz, v, ci, qN, cd, q, bv, dY, vJ, oj, al, eV, hn, bwV, MO, Qr, aW, xs, fO, r, g, iV, t, Vi, ck, bP, wk, oh, m, Xu); #c1(MYOF) c2(NP_038479) c3(myoferlin isoform a) c4(176695, 176696, 176697) c5(176698, 176699, 176700, 176701, 176702, 176703, 176704, 176705, 176706, 176707, 176708, 176709) c6( ) c7(adu, T, cc, aW, xs, aT); #c1(MYOF) c2(NP_579899) c3(myoferlin isoform b) c4(234707) c5(234708, 234709, 234710, 234711, 234712, 234713, 234714, 234715, 234716, 234717, 234718, 234719) c6( ) c7(adu, T, cc, aW, xs, aT); #c1(MYOF) c2(XP_005269750) c3(myoferlin isoform X1) c4(282753, 282754) c5(282755, 282756, 282757, 282758, 282759, 282760, 282761, 282762, 282763, 282764, 282765, 282766) c6( ) c7(adu, T, cc, aW, xs, aT); #c1(MYOG) c2(NP_002470) c3(myogenin) c4(129118) c5(129119, 129120, 129121, 129122, 129123, 129124, 129125) c6( ) c7(bv, hM, cdV, anR, LL, Qr, pd, fO, Ad, kf, v, bl, Xu, Vi, cm, xe, Wq, dR); #c1(MYOM1) c2(NP_003794) c3(myomesin-1 isoform a) c4(140912, 140913) c5(140914, 140915, 140916, 140917, 140918, 140919, 140920, 140921, 140922, 140923, 140924, 140925) c6( ) c7(aPm); #c1(MYOM1) c2(NP_062830) c3(myomesin-1 isoform b) c4(202179) c5(202180, 202181, 202182, 202183, 202184, 202185) c6( ) c7(aPm); #c1(MYOM2) c2(NP_003961) c3(myomesin-2) c4(142479) c5(142480, 142481, 142482, 142483, 142484, 142485) c6( ) c7(T, Ke, fd, bS, ct, sD, ad, hX, cit, ke, zw, pe, pN, gd, aT, ey, kU, vO, tw, afu, qZ, aQk, up, aW, Y, lh, wc, cr, r, lo, CQ, Z, zJ, cis, W, ac, RB, bPc, qr, AH, gZ, bzm, azy, HE, bH, aMk); #c1(MYOT) c2(NP_001129412) c3(myotilin isoform b) c4( ) c5(51462, 51463, 51464, 51465, 51466) c6( ) c7(md, cc, no, ciu, Ws, Am, xs, aWn, oj, bt, km, akB, civ, K, bSc, bX, bl, alj, bgM, aTm); #c1(MYOT) c2(NP_001287840) c3(myotilin isoform c) c4( ) c5(112993) c6( ) c7(md, cc, no, ciu, Ws, Am, xs, aWn, oj, bt, km, akB, civ, K, bSc, bX, bl, alj, bgM, aTm); #c1(MYOT) c2(NP_006781) c3(myotilin isoform a) c4(168013, 168014) c5(168015, 168016, 168017, 168018, 168019, 168020) c6( ) c7(md, cc, no, ciu, Ws, Am, xs, aWn, oj, bt, km, akB, civ, K, bSc, bX, bl, alj, bgM, aTm); #c1(MYOZ1) c2(NP_067068) c3(myozenin-1) c4(208804) c5(208805, 208806, 208807, 208808, 208809, 208810) c6( ) c7(mx, km, mo); #c1(MYOZ2) c2(NP_057683) c3(myozenin-2) c4(192326) c5(192327, 192328, 192329, 192330, 192331, 192332) c6( ) c7(cc, Am, mx, km, oj, uY, vc, ciw, fy); #c1(MYOZ3) c2(XP_011536006) c3(myozenin-3 isoform X1) c4(333266) c5(333267, 333268, 333269, 333270, 333271, 333272, 333273, 333274, 333275, 333276) c6( ) c7(km); #c1(MYPN) c2(NP_001243196) c3(myopalladin isoform a) c4(90099, 90100, 90101) c5(90102, 90103, 90104, 90105, 90106, 90107, 90108, 90109, 90110, 90111, 90112, 90113, 90114, 90115, 90116, 90117, 90118, 90119) c6( ) c7(AX, cc, mx, vc, ciX, K, vd, mo, cau); #c1(MYPN) c2(NP_001243197) c3(myopalladin isoform b) c4(90120, 90121) c5( ) c6( ) c7(AX, cc, mx, vc, ciX, K, vd, mo, cau); #c1(MYPN) c2(XP_006718106) c3(myopalladin isoform X2) c4( ) c5(291508, 291509, 291510, 291511, 291512, 291513, 291514, 291515, 291516, 291517, 291518, 291519) c6( ) c7(AX, cc, mx, vc, ciX, K, vd, mo, cau); #c1(MYRF) c2(NP_001120864) c3(myelin regulatory factor isoform 2 precursor) c4(44865) c5(44866, 44867, 44868, 44869, 44870, 44871) c6( ) c7(v); #c1(MYRF) c2(NP_037411) c3(myelin regulatory factor isoform 1) c4(175367) c5(175368, 175369, 175370, 175371, 175372, 175373) c6( ) c7(v); #c1(MYRF) c2(XP_011543536) c3(myelin regulatory factor isoform X8) c4(342457) c5(342458, 342459, 342460, 342461, 342462, 342463) c6( ) c7(v); #c1(MYRFL) c2(XP_011537358) c3(myelin regulatory factor-like protein isoform X9) c4(335044) c5(335045, 335046, 335047, 335048, 335049, 335050) c6( ) c7(cd, alv, hH); #c1(MYRIP) c2(NP_001271352) c3(rab effector MyRIP isoform a) c4(103928) c5(103929, 103930, 103931, 103932, 103933, 103934, 103935, 103936, 103937, 103938, 103939, 103940, 103941, 103942, 103943, 103944) c6( ) c7(ciy, aaw, d, W, fq); #c1(MYRIP) c2(NP_001271354) c3(rab effector MyRIP isoform c) c4(103945, 103946) c5(103947, 103948, 103949, 103950, 103951, 103952) c6( ) c7(ciy, aaw, d, W, fq); #c1(MYT1) c2(NP_004526) c3(myelin transcription factor 1) c4(147247, 147248, 147249) c5(147250, 147251, 147252, 147253, 147254, 147255, 147256, 147257, 147258, 147259, 147260, 147261, 147262, 147263, 147264, 147265, 147266, 147267, 147268) c6( ) c7(bmQ, nM, aT, eo, ci, nA, V); #c1(MYT1L) c2(NP_001289981) c3(myelin transcription factor 1-like protein isoform 1) c4(114933) c5(114934, 114935, 114936, 114937, 114938, 114939, 114940) c6( ) c7(bO, aA, aF, v, Z, Fc); #c1(MYT1L) c2(NP_055840) c3(myelin transcription factor 1-like protein isoform 2) c4(183819, 183820) c5(183821, 183822, 183823, 183824, 183825, 183826, 183827, 183828, 183829, 183830, 183831, 183832) c6( ) c7(bO, aA, aF, v, Z, Fc); #c1(MYT1L) c2(XP_011508628) c3(myelin transcription factor 1-like protein isoform X4) c4(299243) c5( ) c6( ) c7(bO, aA, aF, v, Z, Fc); #c1(MYT1L) c2(XP_011508631) c3(myelin transcription factor 1-like protein isoform X7) c4( ) c5(299244, 299245, 299246, 299247, 299248, 299249) c6( ) c7(bO, aA, aF, v, Z, Fc); #c1(MYZAP) c2(NP_001018110) c3(myocardial zonula adherens protein isoform 1 precursor) c4(27341, 27342, 27343) c5(27344, 27345, 27346, 27347, 27348, 27349, 27350, 27351, 27352, 27353, 27354, 27355, 27356, 27357, 27358, 27359, 27360, 27361, 27362, 27363, 27364, 27365, 27366, 27367, 27368, 27369, 27370) c6( ) c7(ae, K, ad); #c1(MYZAP) c2(NP_689664) c3(myocardial zonula adherens protein isoform 2) c4(243515, 243516) c5(243517, 243518, 243519, 243520, 243521, 243522, 243523, 243524, 243525, 243526, 243527, 243528, 243529, 243530, 243531, 243532) c6( ) c7(ae, K, ad); #c1(MZB1) c2(NP_057543) c3(marginal zone B- and B1-cell-specific protein precursor) c4(191328, 191329, 191330) c5(191331, 191332, 191333, 191334, 191335, 191336, 191337, 191338, 191339, 191340, 191341, 191342, 191343, 191344, 191345, 191346, 191347, 191348) c6( ) c7(gh, T, aA, fd, KO, aj, v, uu, aY, V, J, ad, aE, bK, K, he, ae, hM, EX, aq, yq, cm, bz, hq, bP, Ei, cj, ei, bt, B); #c1(MZF1) c2(NP_001253962) c3(myeloid zinc finger 1 isoform 2) c4(94245) c5(94246, 94247, 94248, 94249, 94250) c6( ) c7(aq, T, if, B, qN); #c1(MZF1) c2(XP_006723422) c3(myeloid zinc finger 1 isoform X1) c4(295199) c5(295200, 295201, 295202, 295203, 295204, 295205, 295206, 295207) c6( ) c7(aq, T, if, B, qN); #c1(MZF1) c2(XP_011525566) c3(myeloid zinc finger 1 isoform X3) c4( ) c5(319977, 319978, 319979) c6( ) c7(aq, T, if, B, qN); #c1(MZF1) c2(XP_011525568) c3(myeloid zinc finger 1 isoform X4) c4( ) c5(319980) c6( ) c7(aq, T, if, B, qN); #c1(N4BP1) c2(NP_694574) c3(NEDD4-binding protein 1) c4(245302) c5(245303, 245304, 245305, 245306, 245307, 245308) c6( ) c7(y, cj); #c1(N4BP2L1) c2(NP_001073159) c3(NEDD4-binding protein 2-like 1 isoform 2) c4(35654) c5(35655, 35656, 35657, 35658, 35659, 35660) c6( ) c7(dL); #c1(N4BP2L1) c2(NP_001273388) c3(NEDD4-binding protein 2-like 1 isoform 1) c4(104825) c5(104826, 104827, 104828, 104829, 104830, 104831) c6( ) c7(dL); #c1(N4BP2L1) c2(XP_011533604) c3(NEDD4-binding protein 2-like 1 isoform X3) c4(329712) c5( ) c6( ) c7(dL); #c1(N4BP2L1) c2(XP_011533606) c3(NEDD4- binding protein 2-like 1 isoform X5) c4( ) c5(329713) c6( ) c7(dL); #c1(N4BP2L1) c2(XP_011533607) c3(NEDD4-binding protein 2-like 1 isoform X6) c4( ) c5(329714, 329715, 329716, 329717, 329718) c6( ) c7(dL); #c1(N4BP2L2) c2(NP_001265361) c3(NEDD4-binding protein 2-like 2 isoform 3) c4(98377) c5( ) c6( ) c7(gV, hG, r, lQ, Pk, B); #c1(N4BP2L2) c2(NP_055702) c3(NEDD4-binding protein 2-like 2 isoform 2) c4(182886) c5(182887, 182888, 182889, 182890, 182891, 182892) c6( ) c7(gV, hG, r, lQ, Pk, B); #c1(N4BP2L2) c2(NP_149102) c3(NEDD4-binding protein 2-like 2 isoform 1) c4(227165) c5(227166, 227167, 227168, 227169, 227170, 227171, 227172, 227173, 227174, 227175, 227176, 227177) c6( ) c7(gV, hG, r, lQ, Pk, B); #c1(NAA10) c2(NP_001243048) c3(N-alpha-acetyltransferase 10 isoform 2) c4(89667, 89668) c5(89669, 89670, 89671, 89672, 89673, 89674, 89675, 89676, 89677, 89678, 89679, 89680, 89681, 89682) c6( ) c7(ciz, T, kj, Db, aW, apW, aT, Bm, r, adB, ji, apx, q, bJ); #c1(NAA10) c2(NP_003482) c3(N-alpha-acetyltransferase 10 isoform 1) c4(138695) c5(138696, 138697, 138698, 138699, 138700, 138701, 138702, 138703, 138704, 138705) c6( ) c7(ciz, T, kj, Db, aW, apW, aT, Bm, r, adB, ji, apx, q, bJ); #c1(NAA15) c2(NP_476516) c3(N-alpha-acetyltransferase 15, NatA auxiliary subunit) c4(230722, 230723) c5(230724, 230725, 230726, 230727, 230728, 230729, 230730, 230731, 230732, 230733, 230734, 230735) c6( ) c7(T, hd, Db, ci, pk, vt); #c1(NAA15) c2(XP_005263293) c3(N-alpha-acetyltransferase 15, NatA auxiliary subunit isoform X1) c4(278387) c5(278388, 278389, 278390, 278391, 278392, 278393) c6( ) c7(T, hd, Db, ci, pk, vt); #c1(NAA16) c2(NP_001104268) c3(N-alpha-acetyltransferase 16, NatA auxiliary subunit isoform 3) c4(42089) c5(42090, 42091, 42092, 42093, 42094, 42095) c6( ) c7(azs, T, bM, bB, x, gs, eL, aW, NR, g, ba, eM, nB, cX, bcl, k, m, NT, n, cQ, aj, fr, amJ, h, dL, hB, aOX, fe, Qy, at, it, B, bL, bll, aA, cz, q, J, eA, XX, nv, aT, sX, ae, bkl, avV, s, nO, bg, r, eS, en, cT, iQ, ig, ciA, bt, blu, hS, gx, iW, ha, jh, ad, kL, hX, dP, l, hd, NS, oW, io, BO, ff, eZ, Ej, aF, bz, bF, apM, bE, X, pP, i, hE, iu, y); #c1(NAA16) c2(NP_078837) c3(N-alpha-acetyltransferase 16, NatA auxiliary subunit isoform 1) c4(215661) c5(215662, 215663, 215664, 215665, 215666, 215667) c6( ) c7(azs, T, bM, bB, x, gs, eL, aW, NR, g, ba, eM, nB, cX, bcl, k, m, NT, n, cd, aj, fr, amJ, h, dL, hB, aOX, fe, Qy, at, it, B, bL, bll, aA, cz, q, J, eA, XX, nv, aT, sX, ae, bkl, avV, s, nO, bg, r, eS, en, cT, iQ, ig, ciA, bt, blu, hS, gx, iW, ha, jh, ad, kL, hX, dP, l, hd, NS, oW, io, BO, ff, eZ, Ej, aF, bz, bF, apM, bE, X, pP, i, hE, iu, y); #c1(NAA16) c2(XP_011533530) c3(N-alpha-acetyltransferase 16, NatA auxiliary subunit isoform X4) c4(329652) c5(329653, 329654, 329655, 329656, 329657, 329658) c6( ) c7(azs, T, bM, bB, x, gs, eL, aW, NR, g, ba, eM, nB, cX, bcl, k, m, NT, n, cQ, aj, fr, amJ, h, dL, hB, aOX, fe, Qy, at, it, B, bL, bll, aA, cz, q, J, eA, XX, nv, aT, sX, ae, bkl, avV, s, nO, bg, r, eS, en, cT, iQ, ig, ciA, bt, blu, hS, gx, iW, ha, jh, ad, KL, hX, dP, l, hd, NS, oW, io, BO, ff, eZ, Ej, aF, bz, bF, apM, bE, X, pP, i, hE, iu, y); #c1(NAA20) c2(NP_057184) c3(N-alpha-acetyltransferase 20 isoform a) c4(189349) c5(189350, 189351, 189352, 189353, 189354, 189355) c6( ) c7(K); #c1(NAA20) c2(NP_852668) c3(N-alpha-acetyltransferase 20 isoform b) c4(255118) c5(255119, 255120, 255121, 255122, 255123, 255124) c6( ) c7(K); #c1(NAA20) c2(NP_852669) c3(N-alpha-acetyltransferase 20 isoform c) c4(255125) c5(255126, 255127, 255128, 255129, 255130, 255131) c6( ) c7(K); #c1(NAA25) c2(NP_079229) c3(N-alpha-acetyltransferase 25, NatB auxiliary subunit) c4(217548) c5(217549, 217550, 217551, 217552, 217553, 217554) c6( ) c7(bL, T, cz, n, cl, hy, j, ci, C, AO, ai, eA, o, aE, aT, zy, af, A, qt, aW, cr, r, eZ, tv, tu, iK, y, aml); #c1(NAA30) c2(NP_001011713) c3 (N-alpha-acetyltransferase 30) c4(24775, 24776) c5(24777, 24778, 24779, 24780, 24781, 24782, 24783, 24784, 24785, 24786, 24787, 24788) c6( ) c7(W); #c1(NAA35) c2(NP_078911) c3(N-alpha-acetyltransferase 35, NatC auxiliary subunit) c4(216067, 216068, 216069) c5(216070, 216071, 216072, 216073, 216074, 216075, 216076, 216077, 216078, 216079, 216080, 216081, 216082, 216083, 216084, 216085, 216086, 216087) c6( ) c7(T, it); #c1(NAA40) c2(NP_001287729) c3(N-alpha-acetyltransferase 40 isoform 2) c4( ) c5(112678, 112679, 112680, 112681, 112682, 112683, 112684, 112685, 112686, 112687, 112688, 112689) c6( ) c7(aq, B); #c1(NAA40) c2(NP_079047) c3(N-alpha-acetyltransferase 40 isoform 1) c4(216744, 216745) c5( ) c6( ) c7(aq, B); #c1(NAA50) c2(NP_079422) c3(N-alpha-acetyltransferase 50) c4(218050, 218051) c5(218052, 218053, 218054, 218055, 218056, 218057) c6( ) c7(cF, xU, Z, ob, aFa, bhl, aql, bS, ct, AC, aDC, fu); #c1(NAA60) c2(NP_001077069) c3(N-alpha-acetyltransferase 60) c4(37640) c5(37641, 37642, 37643, 37644, 37645, 37646, 37647, 37648, 37649, 37650, 37651) c6( ) c7(oe); #c1(NAAA) c2(NP_001035861) c3(N-acylethanolamine-hydrolyzing acid amidase isoform 2 precursor) c4( ) c5(33284, 33285, 33286, 33287, 33288, 33289, 33290, 33291, 33292, 33293, 33294, 33295, 33296, 33297, 33298, 33299) c6( ) c7(T, arI, aW, e, qr, aT, af, Gc, ad); #c1(NAAA) c2(XP_005262977) c3(N-acylethanolamine-hydrolyzing acid amidase isoform X1) c4(278182, 278183, 278184) c5(278185, 278186, 278187, 278188, 278189, 278190) c6( ) c7(T, arI, aW, e, qr, aT, af, Gc, ad); #c1(NAALAD2) c2(XP_011540854) c3(N-acetylated-alpha-linked acidic dipeptidase 2 isoform X3) c4(339360) c5(339361, 339362, 339363, 339364, 339365, 339366, 339367) c6( ) c7(e); #c1(NAALAD2) c2(XP_011540856) c3(N-acetylated-alpha-linked acidic dipeptidase 2 isoform X6) c4(339368) c5(339369, 339370, 339371, 339372, 339373) c6( ) c7(e); #c1(NAALADL1) c2(NP_005459) c3 (N-acetylated-alpha-linked acidic dipeptidase-like protein) c4(156354, 156355, 156356) c5(156357, 156358, 156359, 156360, 156361, 156362, 156363, 156364, 156365, 156366, 156367, 156368) c6( ) c7(cm, vA); #c1(NAALADL1) c2(XP_011543008) c3(N-acetylated-alpha-linked acidic dipeptidase-like protein isoform X1) c4( ) c5(341693, 341694, 341695, 341696, 341697, 341698) c6( ) c7(cm, vA); #c1(NAALADL1) c2(XP_011543012) c3(N-acetylated-alpha-linked acidic dipeptidase-like protein isoform X5) c4(341699) c5(341700, 341701, 341702, 341703, 341704, 341705) c6( ) c7(cm, vA); #c1(NAALADL2) c2(NP_996898) c3(inactive N-acetylated-alpha-linked acidic dipeptidase-like protein 2) c4(264864) c5(264865, 264866, 264867, 264868, 264869, 264870) c6( ) c7(aNL, lf); #c1(NAALADL2) c2(XP_011510918) c3 (inactive N-acetylated-alpha-linked acidic dipeptidase-like protein 2 isoform X5) c4(301391) c5(301392) c6( ) c7(aNL, lf); #c1(NAALADL2) c2(XP_011510919) c3 (inactive N-acetylated- alpha-linked acidic dipeptidase-like protein 2 isoform X6) c4( ) c5(301393, 301394, 301395, 301396, 301397) c6( ) c7(aNL, If); #c1(NAB1) c2(NP_005957) c3(NGF1-A-binding protein 1) c4(160719, 160720, 160721, 160722) c5(160723, 160724, 160725, 160726, 160727, 160728, 160729, 160730, 160731, 160732, 160733) c6( ) c7(oe, bc, bkW); #c1(NAB1) c2(XP_005246643) c3(NGF1-A-binding protein 1 isoform X2) c4(267251, 267252) c5(267253, 267254, 267255, 267256, 267257, 267258) c6( ) c7(oe, bc, bkW); #c1(NAB1) c2(XP_005246644) c3(NGF1-A-binding protein 1 isoform X5) c4(267259, 267260, 267261) c5(267262, 267263, 267264, 267265, 267266, 267267, 267268, 267269, 267270, 267271, 267272, 267273, 267274, 267275, 267276, 267277, 267278, 267279, 267280, 267281, 267282, 267283, 267284, 267285, 267286, 267287, 267288, 267289, 267290, 267291, 267292, 267293, 267294, 267295, 267296, 267297, 267298, 267299, 267300, 267301, 267302) c6( ) c7(oe, bc, bkW); #c1(NAB2) c2(NP_005958) c3(NGF1-A-binding protein 2) c4(160734) c5(160735, 160736, 160737, 160738, 160739, 160740, 160741, 160742) c6( ) c7(ae, bkW, aW, au, ff, aT, en, pk, bz, ad); #c1(NAB2) c2(XP_005268951) c3(NGF1-A-binding protein 2 isoform X1) c4(282213, 282214) c5(282215, 282216, 282217, 282218, 282219, 282220, 282221, 282222, 282223, 282224, 282225) c6( ) c7(ae, bkW, aW, au, ff, al, en, pk, bz, ad); #c1(NABP1) c2(NP_001026886) c3(SOSS complex subunit B2 isoform 1) c4(29844, 29845) c5(29846, 29847, 29848, 29849, 29850, 29851, 29852, 29853) c6( ) c7(it); #c1(NABP1) c2(NP_001241665) c3(SOSS complex subunit B2 isoform 2) c4( ) c5(89028, 89029, 89030, 89031, 89032, 89033, 89034, 89035) c6( ) c7(it); #c1(NABP2) c2(NP_076973) c3(SOSS complex subunit BI) c4( ) c5(214529, 214530, 214531, 214532, 214533, 214534, 214535, 214536, 214537, 214538, 214539, 214540, 214541, 214542, 214543, 214544, 214545, 214546, 214547, 214548, 214549) c6( ) c7(T, aKO, B, K); #c1(NABP2) c2(XP_005269203) c3(SOSS complex subunit B1 isoform X2) c4(282476, 282477, 282478) c5(282479, 282480, 282481, 282482) c6( ) c7(T, aKO, B, K); #c1(NABP2) c2(XP_005269204) c3(SOSS complex subunit B1 isoform X3) c4( ) c5(282483) c6( ) c7(T, aKO, B, K); #c1(NACA) c2(NP_001106674) c3(nascent polypeptide-associated complex subunit alpha isoform a) c4(42574) c5(42575, 42576, 42577, 42578, 42579, 42580) c6( ) c7(f, pq, af); #c1(NACA) c2(XP_006719476) c3(nascent polypeptide-associated complex subunit alpha isoform X2) c4( ) c5(292518, 292519, 292520, 292521) c6( ) c7(f, pq, af); #c1(NACA) c2(XP_006719484) c3(nascent polypeptide-associated complex subunit alpha isoform X1) c4(292522, 292523, 292524) c5(292525, 292526, 292527, 292528, 292529, 292530, 292531, 292532, 292533, 292534, 292535, 292536, 292537, 292538, 292539, 292540, 292541, 292542, 292543, 292544, 292545, 292546, 292547, 292548, 292549, 292550, 292551, 292552, 292553, 292554) c6( ) c7(f, pq, af); #c1(NACA) c2(XP_011536691) c3(nascent polypeptide-associated complex subunit alpha isoform X3) c4(334225) c5(334226, 334227, 334228, 334229, 334230, 334231, 334232, 334233, 334234, 334235, 334236, 334237, 334238, 334239) c6( ) c7(f, pq, af); #c1(NACC1) c2(XP_005259778) c3(nucleus accumbens-associated protein 1 isoform X1) c4(276298) c5(276299, 276300, 276301, 276302, 276303, 276304, 276305) c6( ) c7(T, auY, Y, if, ff, cm, n, agF, np, ci, m, en, qN, t, g, Tj); #c1(NACC2) c2(XP_011516523) c3(nucleus accumbens-associated protein 2 isoform X1) c4(309934) c5(309935, 309936, 309937, 309938, 309939, 309940, 309941, 309942, 309943, 309944, 309945) c6( ) c7(ac, Ea); #c1(NADSYN1) c2(NP_060631) c3(glutamine-dependent NAD(+) synthetase) c4(196912, 196913) c5(196914, 196915, 196916, 196917, 196918, 196919, 196920, 196921, 196922, 196923, 196924, 196925) c6( ) c7(A, ac, e, r, dy, j, y, q); #c1(NAE1) c2(NP_001018169) c3(NEDD8-activating enzyme E1 regulatory subunit isoform b) c4(27507, 27508) c5(27509, 27510, 27511, 27512, 27513, 27514) c6( ) c7(T, s, pf, aA, r, aF, q, g, YQ, f, X); #c1(NAE1) c2(NP_001273429) c3(NEDD8-activating enzyme E1 regulatory subunit isoform d) c4( ) c5(104880) c6( ) c7(T, s, pf, aA, r, aF, q, g, YQ, f, X); #c1(NAE1) c2(NP_003896) c3(NEDD8-activating enzyme E1 regulatory subunit isoform a) c4(141940) c5(141941, 141942, 141943, 141944, 141945, 141946) c6( ) c7(T, s, pf, aA, r, aF, q, g, YQ, f, X); #c1(NAE1) c2(XP_011521724) c3(NEDD8-activating enzyme E1 regulatory subunit isoform X1) c4( ) c5(316445, 316446, 316447, 316448, 316449) c6( ) c7(T, s, pf, aA, r, aF, q, g, YA, f, X); #c1(NAF1) c2(NP_001122403) c3(H/ACA ribonucleoprotein complex non-core subunit NAF1 isoform b) c4(46789) c5(46790, 46791, 46792, 46793, 46794, 46795) c6( ) c7(sb, y, hl, aWF); #c1(NAF1) c2(NP_612395) c3(H/ACA ribonucleoprotein complex non-core subunit NAF1 isoform a) c4(236363) c5(236364, 236365, 236366, 236367, 236368, 236369) c6( ) c7(sb, y, hl, aWF); #c1(NAGA) c2(XP_005261673) c3(alpha-N-acetylgalactosaminidase isoform X1) c4(277578) c5(277579, 277580, 277581, 277582, 277583, 277584, 277585, 277586, 277587, 277588, 277589, 277590, 277591, 277592) c6( ) c7(aR, cc, ciD, ciC, ds, hO, ciB, dr, aya); #c1(NAGK) c2(NP_060037) c3(N-acetyl-D-glucosamine kinase) c4(193817, 193818) c5(193819, 193820, 193821, 193822, 193823, 193824, 193825, 193826, 193827, 193828, 193829, 193830) c6( ) c7(ad); #c1(NAGLU) c2(NP_000254) c3(alpha-N-acetylglucosaminidase precursor) c4(5825) c5(5826, 5827, 5828, 5829, 5830, 5831) c6( ) c7(jf, iF, lE, arl, Yc, tb, aBU, kg, aAh, awd, KW, jN, bw, lo, Lq, ww, nA, R, cm, dx, ad); #c1(NAGLU) c2(XP_011523142) c3(alpha-N-acetylglucosaminidase isoform X2) c4(317681) c5(317682, 317683, 317684, 317685, 317686, 317687) c6( ) c7(jf, iF, lE, arI, Yc, tb, aBU, kg, aAh, awd, KW, jN, bw, lo, Lq, ww, nA, R, cm, dx, ad); #c1(NAGPA) c2(NP_057340) c3 (N-acetylglucosamine-I-phosphodiester alpha-N-acetylglucosaminidase precursor) c4(190185, 190186) c5(190187, 190188, 190189, 190190, 190191, 190192, 190193, 190194, 190195, 190196, 190197, 190198, 190199) c6( ) c7(aQQ); #c1(NAGS) c2(NP_694551) c3(N-acetylglutamate synthase, mitochondrial) c4(245129, 245130) c5(245131, 245132, 245133, 245134, 245135, 245136, 245137, 245138, 245139, 245140, 245141, 245142, 245143, 245144, 245145, 245146, 245147, 245148, 245149, 245150, 245151, 245152, 245153, 245154, 245155, 245156, 245157, 245158, 245159, 245160, 245161, 245162, 245163, 245164, 245165, 245166, 245167, 245168, 245169, 245170, 245171, 245172, 245173, 245174, 245175, 245176, 245177, 245178, 245179, 245180, 245181, 245182, 245183, 245184, 245185, 245186, 245187, 245188, 245189, 245190, 245191, 245192, 245193, 245194, 245195, 245196, 245197, 245198, 245199, 245200, 245201, 245202, 245203, 245204, 245205, 245206, 245207, 245208, 245209, 245210, 245211, 245212, 245213, 245214, 245215, 245216, 245217, 245218, 245219, 245220, 245221, 245222, 245223, 245224, 245225, 245226, 245227, 245228, 245229, 245230, 245231, 245232, 245233, 245234, 245235, 245236, 245237, 245238, 245239, 245240, 245241, 245242, 245243, 245244, 245245, 245246, 245247, 245248, 245249, 245250, 245251, 245252, 245253, 245254, 245255, 245256, 245257, 245258, 245259, 245260, 245261, 245262, 245263, 245264, 245265, 245266, 245267, 245268, 245269, 245270, 245271, 245272, 245273, 245274, 245275) c6(245276) c7(ciE, aip); #c1(NAGS) c2(XP_011522741) c3(N-acetylglutamate synthase, mitochondrial isoform X2) c4(317268) c5(317269, 317270, 317271, 317272, 317273) c6( ) c7(ciE, aip); #c1(NAIP) c2(NP_004527) c3(baculoviral IAP repeat-containing protein 1 isoform 1) c4(147269) c5(147270, 147271, 147272, 147273, 147274, 147275, 147276, 147277, 147278, 147279) c6( ) c7(oO, ck, T, d, apq, c, apI, arX, aT, kf, cy, hO, bw, bl, g, rz, V, ae, ad); #c1(NAIP) c2(NP_075043) c3(baculoviral IAP repeat-containing protein 1 isoform 2) c4(213532) c5(213533, 213534, 213535, 213536, 213537, 213538, 213539, 213540, 213541, 213542) c6( ) c7(oO, ck, T, d, apq, c, apl, arX, aT, kf, cy, hO, bw, bl, g, rz, V, ae, ad); #c1(NALCN) c2(XP_011519369) c3(sodium leak channel non-selective protein isoform X2) c4(314017, 314018) c5(314019, 314020) c6( ) c7(cd, he, bK, ciF); #c1(NALCN) c2(XP_011519370) c3(sodium leak channel non-selective protein isoform X1) c4( ) c5(314021, 314022, 314023, 314024, 314025) c6( ) c7(cd, he, bk, ciF); #c1(NALCN) c2(XP_011519371) c3(sodium leak channel non-selective protein isoform X3) c4(314026, 314027) c5( ) c6( ) c7(cd, he, bK, ciF); #c1(NALCN) c2(XP_011519372) c3(sodium leak channel non-selective protein isoform X4) c4( ) c5(314028, 314029, 314030, 314031, 314032, 314033, 314034, 314035, 314036, 314037, 314038, 314039, 314040, 314041, 314042, 314043, 314044, 314045) c6( ) c7(cd, he, bK, ciF); #c1(NAMPT) c2(XP_005250157) c3(nicotinamide phosphoribosyltransferase isoform X1) c4(270107, 270108, 270109, 270110, 270111, 270112) c5(270113, 270114, 270115, 270116, 270117, 270118, 270119, 270120, 270121, 270122, 270123, 270124, 270125, 270126, 270127, 270128, 270129, 270130, 270131, 270132, 270133, 270134, 270135, 270136) c6( ) c7(bL, T, dD, fR, aZ, ea, al, EQ, fF, fd, fg, afK, dG, j, aj, C, v, eq, JW, ai, J, eA, GK, BJ, xE, aE, M, aT, cy, dL, ey, K, uV, bq, Qe, qM, ji, eL, d, aW, fJ, fO, fc, fe, iV, fq, ek, ck, eM, iT, mg, mf, aS, dy, fD, ei, y, bt); #c1(NAMPT) c2(XP_011514009) c3(nicotinamide phosphoribosyltransferase isoform X2) c4( ) c5(306414, 306415, 306416, 306417, 306418, 306419, 306420, 306421, 306422, 306423, 306424, 306425) c6( ) c7(bL, T, dD, fR, aZ, ea, al, EQ, fF, fd, fg, afK, dG, j, aj, C, v, eq, JW, ai, J, eA, GK, eJ, xE, aE, M, aT, cy, dL, ey, K, uV, bq, Qe, qM, ji, eL, d, aW, fJ, fO, fc, fe, iV, fq, ek, ck, eM, iT, mg, mf, aS, dy, fD, ei, y, bt); #c1(NANOG) c2(NP_001284627) c3(homeobox protein NANOG isoform 2) c4(112159, 112160) c5(112161, 112162, 112163, 112164, 112165, 112166, 112167, 112168, 112169, 112170, 112171, 112172) c6( ) c7(T, wu, if, oe, jh, fZ, bB, Hn, ci, qN, q, ad, eA, BI, aoc, aT, rO, aAE, wm, nJ, ae, d, ZH, gP, B, vT, aW, CN, Lf, ff, r, aq, en, bU, as, t, iR, ck, bE, eD, Lb, m, KY, X); #c1(NANOG) c2(NP_079141) c3(homeobox protein NANOG isoform 1) c4(217215) c5(217216, 217217, 217218, 217219, 217220, 217221, 217222, 217223, 217224, 217225, 217226, 217227, 217228) c6( ) c7(T, wu, if, oe, jh, fZ, bB, Hn, ci, qN, q, ad, eA, BI, aoc, aT, rO, aAE, wm, nJ, ae, d, ZH, gP, B, vT, aW, CN, Lf, ff, r, aq, en, bU, as, t, iR, ck, bE, eD, Lb, m, KY, X); #c1(NANOG) c2(XP_011519154) c3(homeobox protein NANOG isoform X3) c4( ) c5(313755) c6( ) c7(T, wu, if, oe, jh, fZ, bB, Hn, ci, qN, q, ad, eA, Bl, aoc, aT, rO, aAE, wm, nJ, ae, d, ZH, gP, B, vT, aW, CN, Lf, ff, r, aq, en, bU, as, t, iR, ck, bE, eD, Lb, m, KY, X); #c1(NANOS1) c2(NP_955631) c3(nanos homolog 1) c4(261911, 261912) c5(261913, 261914, 261915, 261916, 261917, 261918, 261919, 261920, 261921, 261922, 261923, 261924) c6( ) c7(tJ, bM, eR, qb, iW, ago, uG, yY, cz, bS, gf, ct, fr, bzq, Jt, V, aY, cd, ciG, eA, ip, HF, aE, aT, K, he, qD, rz, aW, Y, ff, HL, Ku, en, Z, es, pH, cT, ac, eM, bjL, fy, xg, ao); #c1(NANOS2) c2(NP_001025032) c3(nanos homolog 2) c4(29239) c5(29240, 29241, 29242, 29243, 29244, 29245) c6( ) c7(ID, bM, T, ciH, gU, dD, ea, jx, bB, n, tc, auq, eq, C, eg, eh, q, aR, ad, ai, F, oW, aT, dV, io, dL, vd, K, hB, H, ae, af, ao, VF, eL, ly, A, zH, dH, aW, wf, hl, cr, r, eZ, aq, lo, bz, pb, lx, bF, pu, ac, c, nM, bE, cj, aAl, amb, y, aen, B, hv); #c1(NANOS3) c2(NP_001092092) c3(nanos homolog 3) c4(39310, 39311) c5(39312, 39313, 39314, 39315, 39316, 39317, 39318, 39319, 39320, 39321, 39322, 39323) c6( ) c7(P, bL, bM, dD, gU, pG, lD, Zl, qb, aZ, ea, e, pf, aw, bO, cz, sM, cR, bS, j, ct, gf, ci, ur, uu, aY, v, cd, ad, ip, x, aif, vJ, iI, M, axc, aT, eq, rO, dL, bk, K, dQ, ae, bPQ, eL, hm, uX, ly, A, dH, cW, asP, aW, aeS, eR, dw, Z, zZ, vo, pb, qa, lx, adA, ayt, ac, cA, nM, wE, mG, mx, U, vy, wR, ro, aS, k, dy, fD, ei, lC, sZ, y); #c1(NANS) c2(NP_061819) c3(sialic acid synthase) c4(200490) c5(200491, 200492, 200493, 200494, 200495, 200496) c6( ) c7(T, sw, ha, aA, bS, ct, iJ, yg, h, cU, d, ff, eH, aF, en, eF, cm, Z, iK, QG, au, QC, AC, cil, nz, bt); #c1(NANS) c2(XP_011517089) c3(sialic acid synthase isoform X1) c4(310623, 310624) c5( ) c6( ) c7(T, sw, ha, aA, bS, ct, iJ, yg, h, c, d, ff, eH, aF, en, eF, cm, Z, iK, QG, au, QC, AC, cil, nz, bt); #c1(NANS) c2(XP_011517090) c3(sialic acid synthase isoform X2) c4( ) c5(310625, 310626, 310627, 310628, 310629, 310630, 310631, 310632, 310633, 310634, 310635, 310636) c6( ) c7(T, sw, ha, aA, bS, ct, iJ, yg, h, cu, d, ff, eH, aF, en, eF, cm, Z, iK, QG, au, QC, AC, cil, nz, bt); #c1(NAP1L1) c2(NP_004528) c3(nucleosome assembly protein 1-like 1) c4(147280, 147281, 147282) c5(147283, 147284, 147285, 147286, 147287, 147288, 147289, 147290, 147291, 147292, 147293, 147294, 147295, 147296, 147297, 147298, 147299, 147300, 147301, 147302, 147303, 147304, 147305, 147306, 147307, 147308) c6( ) c7(T, d, af, dB, aW, eL, cr, aT, n, aTl, tv, tu, K, C, EY, dL, cm, ae, q, ad, amL); #c1(NAP1L1) c2(XP_006719488) c3(nucleosome assembly protein 1-like 1 isoform X7) c4(292555) c5(292556, 292557, 292558, 292559, 292560, 292561, 292562, 292563, 292564, 292565, 292566, 292567) c6( ) c7(T, d, af, dB, aW, eL, cr, aT, n, aTl, tv, tu, K, C, EY, dL, cm, ae, q, ad, amL); #c1(NAP1L1) c2(XP_011536692) c3(nucleosome assembly protein 1-like 1 isoform X2) c4(334240, 334241, 334242) c5(334243, 334244, 334245, 334246) c6( ) c7(T, d, af, dB, aW, eL, cr, aT, n, aTl, tv, tu, K, C, EY, dL, cm, ae, q, ad, amL); #c1(NAP1L3) c2(NP_004529) c3(nucleosome assembly protein 1-like 3) c4(147309, 147310) c5(147311, 147312, 147313, 147314, 147315, 147316, 147317, 147318, 147319, 147320, 147321, 147322) c6( ) c7(ae, ad); #c1(NAP1L4) c2(NP_005960) c3(nucleosome assembly protein 1-like 4) c4(160756, 160757, 160758, 160759, 160760, 160761, 160762, 160763, 160764) c5(160765, 160766, 160767, 160768, 160769, 160770, 160771, 160772, 160773, 160774, 160775, 160776, 160777, 160778, 160779, 160780, 160781, 160782, 160783, 160784, 160785, 160786, 160787, 160788, 160789, 160790, 160791, 160792, 160793, 160794, 160795, 160796, 160797, 160798, 160799, 160800, 160801, 160802, 160803, 160804, 160805, 160806, 160807, 160808, 160809, 160810, 160811, 160812, 160813, 160814, 160815, 160816, 160817, 160818) c6( ) c7(n, er, K); #c1(NAP1L5) c2(NP_715638) c3(nucleosome assembly protein 1-like 5) c4(246684, 246685) c5(246686, 246687, 246688, 246689, 246690, 246691, 246692, 246693, 246694, 246695, 246696, 246697) c6( ) c7(v); #c1(NAPA) c2(NP_003818) c3(alpha-soluble NSF attachment protein) c4(141119) c5(141120, 141121, 141122, 141123, 141124, 141125) c6( ) c7(ul, K, Gx); #c1(NAPEPLD) c2(XP_006725119) c3 (N-acyl-phosphatidylethanolamine-hydrolyzing phospholipase D isoform X1) c4(296573, 296574, 296575) c5(296576, 296577, 296578, 296579, 296580, 296581, 296582, 296583, 296584, 296585, 296586, 296587, 296588, 296589, 296590, 296591, 296592, 296593, 296594, 296595, 296596, 296597, 296598, 296599, 296600, 296601, 296602, 296603, 296604, 296605, 296606, 296607, 296608, 296609, 296610, 296611, 296612) c6( ) c7(hp, W); #c1(NAPG) c2(NP_003817) c3(gamma-soluble NSF attachment protein) c4(141111) c5(141112, 141113, 141114, 141115, 141116, 141117, 141118) c6( ) c7(vr, ciJ, bK); #c1(NAPG) c2(XP_011524057) c3(gamma-soluble NSF attachment protein isoform X2) c4(318648) c5( ) c6( ) c7(vr, ciJ, bK); #c1(NAPG) c2(XP_011524058) c3(gamma-soluble NSF attachment protein isoform X3) c4( ) c5(318649, 318650, 318651, 318652, 318653) c6( ) c7(vr, ciJ, bK); #c1(NAPRT) c2(NP_001273758) c3(nicotinate phosphoribosyltransferase isoform 2) c4(105781) c5(105782, 105783, 105784, 105785, 105786, 105787) c6( ) c7(T, d); #c1(NAPRT) c2(NP_660202) c3(nicotinate phosphoribosyltransferase isoform 1) c4(240510, 240511, 240512) c5(240513, 240514, 240515, 240516, 240517, 240518, 240519, 240520, 240521, 240522, 240523, 240524, 240525, 240526, 240527, 240528, 240529, 240530) c6( ) c7(T, d); #c1(NAPSA) c2(XP_011525842) c3(napsin-A isoform X1) c4(320247) c5(320248, 320249, 320250, 320251, 320252, 320253, 320254, 320255) c6(320256, 320257) c7(T, n, ci, C, lu, VV, eJ, K, af, d, ff, cr, tv, tu, en, g, as, t, bz, m, iu, amL); #c1(NARFL) c2(NP_001291728) c3(cytosolic Fe-S cluster assembly factor NARFL isoform 2) c4( ) c5(116428, 116429, 116430, 116431, 116432, 116433) c6( ) c7(uJ, ac); #c1(NARFL) c2(NP_071938) c3(cytosolic Fe-S cluster assembly factor NARFL isoform 1) c4(212408, 212409, 212410) c5(212411, 212412, 212413, 212414, 212415, 212416, 212417, 212418, 212419, 212420, 212421, 212422) c6( ) c7(uJ, ac); #c1(NARR) c2(NP_001243210) c3(ras-related protein Rab-34, isoform NARR) c4( ) c5(90174) c6( ) c7(aW, aT); #c1(NARS) c2(NP_004530) c3(asparagine-tRNA ligase, cytoplasmic) c4(147323, 147324, 147325, 147326) c5(147327, 147328, 147329, 147330, 147331, 147332, 147333, 147334, 147335, 147336, 147337) c6( ) c7(q, r); #c1(NARS) c2(XP_005266757) c3(asparagine-tRNA ligase, cytoplasmic isoform X1) c4( ) c5(280305, 280306, 280307, 280308, 280309, 280310, 280311, 280312, 280313, 280314, 280315, 280316, 280317) c6( ) c7(q, r); #c1(NAT1) c2(NP_001153646) c3(arylamine N-acetyltransferase 1 isoform a) c4( ) c5(60825, 60826, 60827, 60828, 60829, 60830, 60831, 60832, 60833, 60834, 60835, 60836, 60837, 60838, 60839, 60840, 60841, 60842, 60843, 60844, 60845, 60846, 60847) c6( ) c7(gx, azs, T, GI, bM, fR, s, aC, fV, OI, id, jx, aCw, iW, aA, bB, fd, hy, jE, bX, fw, q, ad, hX, Na, XW, av, jz, ib, aE, amJ, NS, aT, Nc, dL, lQ, Od, j, ae, af, bkl, eL, aAX, ny, avV, BN, B, s, BA, aW, AQ, ml, ff, Sn, ge, r, aq, cr, aF, eZ, en, g, iV, cm, iK, bz, bJ, ck, cT, apM, lp, bE, dJ, X, cj, uo, aSp, ut, oQ, bt, ne, blu); #c1(NAT1) c2(NP_001278891) c3(arylamine N-acetyltransferase 1 isoform b) c4(110819, 110820, 110821) c5(110822, 110823, 110824, 110825, 110826, 110827, 110828, 110829, 110830, 110831, 110832, 110833, 110834) c6( ) c7(gx, azs, T, GI, bM, fR, sQ, aC, fV, OI, id, jx, aCw, iW, aA, bB, fd, hy, jE, bX, fw, q, ad, hX, Na, XW, av, jz, ib, aE, amJ, NS, aT, Nc, dL, lQ, Od, jj, ae, af, bkl, eL, aAX, ny, avV, BN, B, s, BA, aW, AQ, mQ, ff, Sn, ge, r, aq, cr, aF, eZ, en, g, iV, cm, iK, bz, bJ, ck, cT, apM, lp, bE, dJ, X, cj, uo, aSp, ut, od, bt, ne, blu); #c1(NAT10) c2(XP_011518499) c3(N-acetyltransferase 10 isoform X1) c4(312569, 312570) c5(312571, 312572, 312573, 312574, 312575, 312576, 312577, 312578, 312579, 312580, 312581, 312582) c6( ) c7(Zu, TI, vT, HY, TH, TG, ZB, TJ, bE, eH, au, bB, aq, rx, h, fd, eF, y, nJ, B, bc); #c1(NAT14) c2(NP_065111) c3(N-acetyltransferase 14) c4(203973) c5(203974, 203975, 203976, 203977, 203978, 203979, 203980, 203981, 203982, 203983) c6( ) c7(Rq); #c1(NAT2) c2(XP_011542660) c3(arylamine N-acetyltransferase 2 isoform X1) c4(341215) c5(341216, 341217, 341218, 341219, 341220, 341221) c6( ) c7(azs, atT, T, bM, aC, fR, fV, cik, al, fd, bB, lj, ci, ip, x, XW, Na, iI, fS, ja, gs, hH, eL, aAX, aW, hl, NR, g, as, eE, uC, ba, y, eM, nB, lp, iT, U, cj, uo, cX, bcl, k, m, iu, dD, sl, sw, OI, ht, NT, n, cQ, aj, fr, qN, jz, cc, amJ, h, Gw, dL, hB, aDX, ny, A, sp, fJ, AQ, ge, fe, aq, bJ, Qy, W, dB, at, uz, ut, it, B, bL, aCw, bll, dk, aA, cz, hy, aNG, j, q, J, eA, XX, nv, aT, Nc, an, sX, ae, bkl, af, ne, avV, s, nO, bg, r, eS, en, t, ck, cT, iI, ig, mG, ciA, bt, blu, vi, hS, gx, Gl, id, iW, ha, jh, Gn, jE, asl, eq, ad, kL, hX, dP, av, l, hd, ib, Db, NS, oW, io, lQ, XI, BO, hG, cH, ff, Sn, cr, eZ, Ej, aF, iV, gW, ciL, bz, bF, es, apM, atM, bE, dO, pP, aSp, dX, Gu, i, HE, X, rk, BA); #c1(NAT6) c2(NP_001186947) c3(N-acetyltransferase 6 isoform 2) c4( ) c5(78836, 78837, 78838, 78839, 78840, 78841, 78842, 78843, 78844, 78845, 78846, 78847, 78848, 78849, 78850, 78851, 78852, 78853, 78854, 78855, 78856, 78857, 78858, 78859, 78860, 78861, 78862) c6( ) c7(ja); #c1(NAT6) c2(NP_036323) c3 (N-acetyltransferase 6 isoform 1) c4(173009, 173010, 173011, 173012) c5(173013, 173014, 173015, 173016, 173017, 173018, 173019, 173020, 173021, 173022, 173023, 173024) c6( ) c7(ja); #c1(NAT8) c2(NP_003951) c3(N-acetyltransferase 8) c4(142414) c5(142415, 142416, 142417, 142418, 142419, 142420) c6( ) c7(T, eR, uv, ht, cz, aj, fo, q, ad, kL, Na, fU, b, hd, M, aT, dL, hu, ae, af, eL, avV, aW, ff, hl, cr, r, eZ, Ej, fe, aF, en, dw, iK, bz, ck, ac, iT, nB, hO, bcl, dy, i, hE, it, B, y); #c1(NAT8B) c2(NP_057431) c3(putative N-acetyltransferase 8B) c4(190772) c5( ) c6( ) c7(cT, T, af, bE, aW, eL, aT, fQ, cy, r, dL, bB, iV, ae, q, ad); #c1(NAT8L) c2(NP_848652) c3(N-acetylaspartate synthetase) c4(254074, 254075) c5(254076, 254077, 254078, 254079, 254080, 254081, 254082, 254083, 254084, 254085, 254086, 254087) c6( ) c7(ciM); #c1(NAV1) c2(NP_001161210) c3(neuron navigator 1 isoform 2) c4(65496) c5(65497, 65498, 65499, 65500, 65501, 65502, 65503) c6( ) c7(aW, oe, aT); #c1(NAV1) c2(NP_065176) c3(neuron navigator 1 isoform 1) c4(204462) c5(204463, 204464, 204465, 204466, 204467, 204468) c6( ) c7(aW, oe, aT); #c1(NAV1) c2(XP_006711673) c3(neuron navigator 1 isoform X9) c4( ) c5(286209, 286210, 286211, 286212, 286213) c6( ) c7(aW, oe, aT); #c1(NAV1) c2(XP_006711674) c3(neuron navigator 1 isoform X10) c4( ) c5(286214, 286215, 286216, 286217, 286218) c6( ) c7(aW, oe, aT); #c1(NAV1) c2(XP_011508399) c3(neuron navigator 1 isoform X1) c4(299110, 299111) c5(299112, 299113, 299114, 299115, 299116, 299117) c6( ) c7(aW, oe, aT); #c1(NAV1) c2(XP_011508400) c3(neuron navigator 1 isoform X2) c4(299118) c5(299119, 299120, 299121, 299122, 299123, 299124) c6( ) c7(aW, Qe, aT); #c1(NAV1) c2(XP_011508401) c3(neuron navigator 1 isoform X3) c4(299125) c5(299126) c6( ) c7(aW, oe, aT); #c1(NAV1) c2(XP_011508402) c3(neuron navigator 1 isoform X4) c4(299127) c5(299128, 299129, 299130, 299131, 299132, 299133) c6( ) c7(aW, oe, aT); #c1(NAV2) c2(NP_001104488) c3(neuron navigator 2 isoform 3) c4(42096) c5(42097, 42098, 42099, 42100, 42101, 42102) c6( ) c7(bB, cm, q); #c1(NAV2) c2(NP_00104489) c3(neuron navigator 2 isoform 4) c4(42103) c5(42104, 42105, 42106, 42107, 42108, 42109, 42110, 42111, 42112, 42113) c6( ) c7(bB, cm, q); #c1(NAV2) c2(NP_001231892) c3(neuron navigator 2 isoform 5) c4(87671) c5(87672, 87673, 87674, 87675, 87676, 87677) c6( ) c7(bB, cm, q); #c1(NAV2) c2(NP_660093) c3(neuron navigator 2 isoform 2) c4(240349) c5(240350, 240351, 240352, 240353, 240354, 240355) c6( ) c7(bB, cm, q); #c1(NAV2) c2(NP_892009) c3(neuron navigator 2 isoform 1) c4(257570) c5(257571, 257572, 257573, 257574, 257575, 257576) c6( ) c7(bB, cm, q); #c1(NAV2) c2(XP_005253271) c3(neuron navigator 2 isoform X15) c4(272359) c5(272360, 272361, 272362, 272363, 272364, 272365) c6( ) c7(bB, cm, q); #c1(NAV2) c2(XP_006718429) c3(neuron navigator 2 isoform X12) c4(291854) c5(291855, 291856, 291857, 291858, 291859, 291860) c6( ) c7(bB, cm, q); #c1(NAV2) c2(XP_011518750) c3(neuron navigator 2 isoform X9) c4(313146) c5( ) c6( ) c7(bB, cm, q); #c1(NAV2) c2(XP_011518753) c3(neuron navigator 2 isoform X13) c4( ) c5(313147, 313148, 313149, 313150, 313151, 313152) c6( ) c7(bB, cm, q); #c1(NAV3) c2(NP_001019554) c3(neuron navigator 3 isoform 1) c4(27790) c5(27791, 27792, 27793, 27794, 27795, 27796) c6( ) c7(bM, T, eU, d, VR, s, af, aE, xR, aw, r, fr, ci, cm, q); #c1(NAV3) c2(NP_055718) c3(neuron navigator 3 isoform 2) c4(183023, 183024) c5(183025, 183026, 183027, 183028, 183029, 183030, 183031, 183032, 183033, 183034, 183035, 183036) c6( ) c7(bM, T, eU, d, VR, s, af, aE, xR, aw, r, fr, ci, cm, q); #c1(NAV3) c2(XP_006719740) c3(neuron navigator 3 isoform X9) c4( ) c5(292952, 292953, 292954, 292955, 292956, 292957) c6( ) c7(bM, T, eU, d, VR, s, af, aE, xR, aw, r, fr, ci, cm, q); #c1(NAV3) c2(XP_011537243) c3(neuron navigator 3 isoform X3) c4(334974) c5(334975, 334976, 334977, 334978, 334979, 334980) c6( ) c7(bM, T, eL, d, VR, s, af, aE, xR, aw, r, fr, ci, cm, q); #c1(NAV3) c2(XP_011537246) c3(neuron navigator 3 isoform X7) c4(334981) c5(334982, 334983, 334984, 334985, 334986, 334987) c6( ) c7(bM, T, eU, d, VR, s, af, aE, xR, aw, r, fr, ci, cm, q); #c1(NAV3) c2(XP_011537247) c3(neuron navigator 3 isoform X8) c4(334988) c5( ) c6( ) c7(bM, T, eU, d, VR, s, af, aE, xR, aw, r, fr, ci, cm, q); #c1(NBAS) c2(NP_056993) c3(neuroblastoma-amplified sequence) c4(188369) c5(188370, 188371, 188372, 188373, 188374, 188375) c6( ) c7(jf, iF, ciN, cZ, lE, tb, Yc, bYA, aAh, awd, KW, aA, jN, bw, awK, ww, dd, Gp, cm, dx); #c1(NBAS) c2(XP_011508663) c3(neuroblastoma-amplified sequence isoform X5) c4(299278) c5(299279, 299280, 299281, 299282, 299283, 299284) c6( ) c7(jf, iF, ciN, cZ, lE, tb, Yc, bYA, aAh, awd, KW, aA, jN, bw, awK, ww, dd, Gp, cm, dx); #c1(NBEA) c2(NP_001191126) c3(neurobeachin isoform 2) c4( ) c5(81061, 81062, 81063, 81064, 81065) c6( ) c7(T, ac, fd, v, rz, bN); #c1(NBEA) c2(NP_056493) c3(neurobeachin isoform 1) c4(187755, 187756, 187757) c5(187758, 187759, 187760, 187761, 187762, 187763, 187764, 187765, 187766, 187767, 187768, 187769, 187770, 187771, 187772, 187773, 187774) c6( ) c7(T, ac, fd, v, rz, bN); #c1(NBEA) c2(XP_005266404) c3(neurobeachin isoform X1) c4( ) c5(279991, 279992) c6( ) c7(T, ac, fd, v, rz, bN); #c1(NBEA) c2(XP_005266407) c3(neurobeachin isoform X5) c4(279993) c5( ) c6( ) c7(T, ac, fd, v, rz, bN); #c1(NBEA) c2(XP_006719869) c3(neurobeachin isoform X10) c4(293037, 293038) c5(293039, 293040, 293041, 293042, 293043, 293044, 293045, 293046, 293047, 293048, 293049, 293050, 293051, 293052, 293053, 293054, 293055, 293056) c6( ) c7(T, ac, fd, v, rz, bN); #c1(NBEA) c2(XP_011533347) c3(neurobeachin isoform X6) c4(329470) c5( ) c6( ) c7(T, ac, fd, v, rz, bN); #c1(NBEA) c2(XP_011533348) c3(neurobeachin isoform X7) c4(329471) c5(329472, 329473, 329474, 329475, 329476, 329477, 329478, 329479, 329480) c6( ) c7(T, ac, fd, v, rz, bN); #c1(NBEAL1) c2(NP_001107604) c3(neurobeachin-like protein 1) c4(42889, 42890) c5(42891, 42892, 42893, 42894, 42895, 42896, 42897, 42898, 42899, 42900, 42901, 42902) c6( ) c7(eA); #c1(NBEAL2) c2(NP_055990) c3(neurobeachin-like protein 2) c4(184968) c5(184969, 184970, 184971, 184972, 184973, 184974, 184975, 184976, 184977, 184978, 184979, 184980, 184981, 184982, 184983, 184984, 184985, 184986) c6( ) c7(T, xu, r, pg, dX, q); #c1(NBEAL2) c2(XP_005265049) c3(neurobeachin-like protein 2 isoform X4) c4(279411) c5(279412, 279413, 279414, 279415, 279416, 279417) c6( ) c7(T, xu, r, pg, dX, q); #c1(NBEAL2) c2(XP_011531835) c3(neurobeachin-like protein 2 isoform X3) c4(327902) c5(327903, 327904, 327905, 327906, 327907, 327908) c6( ) c7(T, xu, r, pg, dX, q); #c1(NBEAL2) c2(XP_011531836) c3(neurobeachin-like protein 2 isoform X5) c4(327909, 327910) c5( ) c6( ) c7(T, xu, r, pg, dX, q); #c1(NBN) c2(NP_002476) c3(nibrin) c4(129158, 129159) c5(129160, 129161, 129162, 129163, 129164, 129165, 129166, 129167, 129168, 129169, 129170) c6( ) c7(atT, T, aC, if, ci, ip, x, iI, Qk, K, eL, zQ, Cn, aW, g, as, iP, eM, nB, iT, aBO, cj, cX, dW, k, m, aBx, n, fr, nA, qg, Bj, h, eV, hn, dL, Cw, CM, QK, dB, at, WL, eX, aA, v, Qo, V, q, eA, aT, qv, an, ae, jf, qw, kl, wr, Y, r, lo, eS, en, t, iK, ck, sS, Dy, ig, rA, bt, ao, GI, iW, jh, oZ, Gn, bRy, hF, ad, av, avp, d, BN, ff, eZ, aJW, bz, dr, adq, ag, bC, kS, sH, ahZ); #c1(NBN) c2(XP_011515348) c3(nibrin isoform X3) c4( ) c5(308194) c6( ) c7(atT, T, aC, if, ci, ip, x, iI, Qk, K, eL, zQ, Cn, aW, g, as, iP, eM, nB, iT, aBO, cj, cx, dW, k, m, aBx, n, fr, nA, qg, Bj, h, eV, hn, dL, Cw, CM, QK, dB, at, WL, eX, aA, v, Do, V, q, eA, aT, qv, an, ae, jf, qw, kl, wr, Y, r, lo, eS, en, t, iK, ck, sS, Dy, ig, rA, bt, ao, GI, iW, jh, oZ, Gn, bRy, hF, ad, av, avp, d, BN, ff, eZ, aJW, bz, dr, adq, ag, bC, KS, sH, ahZ); #c1(NBPF3) c2(XP_011540583) c3(neuroblastoma breakpoint family member 3 isoform X3) c4(339086, 339087, 339088) c5(339089, 339090, 339091, 339092, 339093) c6( ) c7(hl); #c1(NBR1) c2(NP_001278500) c3(next to BRCA1 gene 1 protein isoform b) c4(109767) c5(109768, 109769, 109770, 109771, 109772, 109773) c6( ) c7(bM, T, kh, zX, n, aj, OH, ci, fr, avu, dP, avp, gJ, ib, aE, Db, avs, aT, BO, aW, ff, Am, en, bjl, cl, t, g, apx, AM, hL, m, avr, iu, hS); #c1(NBR1) c2(NP_001278501) c3(next to BRCA1 gene 1 protein isoform c) c4(109774) c5(109775, 109776, 109777, 109778, 109779, 109780) c6( ) c7(bM, T, kh, zX, n, aj, OH, ci, fr, avu, dP, avp, gJ, ib, aE, Db, avs, aT, BO, aW, ff, Qm, en, bjl, cl, t, g, apx, AM, hL, m, avr, iu, hS); #c1(NBR1) c2(NP_114068) c3(next to BRCA1 gene 1 protein isoform a) c4(222109, 222110, 222111) c5(222112, 222113, 222114, 222115, 222116, 222117, 222118, 222119, 222120, 222121, 222122, 222123, 222124, 222125, 222126, 222127) c6( ) c7(bM, T, kh, zX, n, aj, oH, ci, fr, avu, dP, avp, gJ, ib, aE, Db, avs, aT, BO, aW, ff, Am, en, bjl, cl, t, g, apx, AM, hL, m, avr, iu, hS); #c1(NBR1) c2(XP_006721966) c3(next to BRCA1 gene 1 protein isoform X2) c4( ) c5(294500, 294501, 294502, 294503, 294504, 294505) c6( ) c7(bM, T, kh, zX, n, aj, oH, ci, fr, avu, dP, avp, gJ, ib, aE, Db, avs, aT, BO, aW, ff, Am, en, bjl, cl, t, g, apx, AM, hL, m, avr, iu, hS); #c1(NCALD) c2(XP_011515637) c3(neurocalcin-delta isoform X1) c4(308484) c5(308485, 308486, 308487, 308488, 308489, 308490, 308491, 308492, 308493, 308494, 308495, 308496, 308497, 308498, 308499, 308500, 308501, 308502, 308503, 308504, 308505, 308506, 308507) c6( ) c7(bL, uT); #c1(NCAM1) c2(NP_000606) c3(neural cell adhesion molecule 1 isoform 1 precursor) c4(13687) c5(13688, 13689, 13690, 13691, 13692, 13693) c6( ) c7(T, aC, Tb, aAx, fd, bB, ci, amW, x, bv, aE, pX, K, ajA, yo, g, Fu, cm, bw, mZ, eM, nB, iT, mx, zv, k, Xu, fy, ajm, aiK, lT, aw, TI, cd, ke, ft, no, pe, cc, Xe, ju, gd, kH, h, hn, mY, mV, he, aBl, dx, mU, bZT, AQ, mo, Ca, alj, mX, hq, ej, W, cA, bP, dB, hk, hR, lB, B, mT, fM, lk, dA, jx, DV, mS, ti, j, V, q, er, eA, qK, aT, cy, rO, bK, j, af, jf, iF, hM, s, iU, Y, lo, gm, en, aom, hf, aER, zM, f, Vq, iK, FT, bN, Cv, zJ, ck, nM, iM, nG, km, ciO, bGB, gT, bt, aKB, vi, gx, iW, jh, Hn, wV, pz, hF, dP, av, Tg, hd, F, alp, Db, yt, mW, Dq, oj, oW, vc, vB, iI, MG, uQ, d, je, ue, Gv, ff, eZ, Fq, bz, alm, amS, bE, lI, bC, hE, iu, y); #c1(NCAM1) c2(NP_001070150) c3(neural cell adhesion molecule 1 isoform 3 precursor) c4(34604) c5(34605, 34606, 34607, 34608, 34609, 34610) c6( ) c7(T, aC, Tb, aAx, fd, bB, ci, amW, x, bv, aE, pX, K, ajA, yo, g, Fu, cm, bw, mZ, eM, nB, iT, mx, zv, k, Xu, fy, ajm, aiK, lT, aw, Tl, cd, ke, ft, no, pe, cc, Xe, ju, gd, kH, h, hn, mY, mV, he, aBl, dx, mU, bZT, AQ, mo, Ca, alj, mX, hq, ej, W, cA, bP, dB, hk, hR, lB, B, mT, fM, Ik, dA, jx, DV, mS, ti, j, V, q, er, eA, qK, aT, cy, rD, bK, j, af, jf, iF, hM, s, iL, Y, lo, gm, en, aom, hf, aER, zM, f, Vq, iK, FT, bN, Cv, zJ, ck, nM, iM, nG, km, ciO, bGB, gT, bt, aKB, vi, gx, iW, jh, Hn, wV, pz, hF, dP, av, Tg, hd, F, alp, Db, yt, mW, Dq, oj, oW, vc, vB, iI, MG, uQ, d, je, ue, Gv, ff, eZ, Fq, bz, alm, amS, bE, lI, bC, hE, iu, y); #c1(NCAM1) c2(NP_001229537) c3(neural cell adhesion molecule 1 isoform 4 precursor) c4(84617) c5(84618, 84619, 84620, 84621, 84622, 84623) c6( ) c7(T, aC, Tb, aAx, fd, bB, ci, amW, x, bv, aE, pX, K, ajA, yo, g, Fu, cm, bw, mZ, eM, nB, iT, mx, zv, k, Xu, fy, ajm, aiK, lT, aw, TI, cd, ke, ft, no, pe, cc, Xe, ju, gd, kH, h, hn, mY, mV, he, aBl, dx, mU, bZT, AQ, mo, Ca, alj, mX, hq, ej, W, cA, bP, dB, hk, hR, lB, B, mT, fM, lk, dA, jx, DV, mS, ti, j, V, q, er, eA, qK, aT, cy, rO, bK, j, af, jf, iF, hM, s, iU, Y, lo, gm, en, aom, hf, aER, zM, f, Vq, iK, FT, bN, Cv, zJ, ck, nM, iM, nG, km, ciO, bGB, gT, bt, aKB, vi, gx, iW, jh, Hn, wV, pz, hF, dP, av, Tg, hd, F, alp, Db, yt, mW, Dq, oj, oW, vc, vB, iI, MG, uQ, d, je, ue, Gv, ff, eZ, Fq, bz, alm, amS, bE, lI, bC, hE, iu, y); #c1(NCAM1) c2(NP_851996) c3(neural cell adhesion molecule 1 isoform 2 precursor) c4(254735, 254736, 254737) c5(254738, 254739, 254740, 254741, 254742, 254743, 254744, 254745, 254746, 254747, 254748, 254749, 254750, 254751, 254752, 254753, 254754, 254755) c6( ) c7(T, aC, Tb, aAx, fd, bB, ci, amW, x, bv, aE, pX, K, ajA, yo, g, Fu, cm, bw, mZ, eM, nB, iT, mx, zv, k, Xu, fy, ajm, aiK, lT, aw, TI, cd, ke, ft, no, pe, cc, Xe, ju, gd, kH, h, hn, mY, mV, he, aBl, dx, mU, bZT, AQ, mo, Ca, alj, mX, hq, ej, W, cA, bP, dB, hk, hR, lB, B, mT, fM, lk, dA, jx, DV, mS, ti, j, V, q, er, eA, qK, aT, cy, rO, bK, j, af, jf, iF, hM, s, iU, Y, lo, gm, en, aom, hf, aER, zM, f, Vq, iK, FT, bN, Cv, zJ, ck, nM, iM, nG, km, ciO, bGB, gT, bt, aKB, vi, gx, iW, jh, Hn, wV, pz, hF, dP, av, Tg, hd, F, alp, Db, yt, mW, Dq, oj, oW, vc, vB, iI, MG, uQ, d, je, ue, Gv, ff, eZ, Fq, bz, alm, amS, bE, lI, bC, hE, iu, y); #c1(NCAM2) c2(NP_004531) c3(neural cell adhesion molecule 2 precursor) c4(147338, 147339) c5(147340, 147341, 147342, 147343, 147344, 147345) c6( ) c7(T, W, c, aW, aT, v, f, af); #c1(NCAM2) c2(XP_011527880) c3(neural cell adhesion molecule 2 isoform X2) c4( ) c5(322989, 322990, 322991, 322992, 322993, 322994) c6( ) c7(T, W, c, aW, aT, v, f, af); #c1(NCAM2) c2(XP_011527883) c3(neural cell adhesion molecule 2 isoform X5) c4(322995) c5( ) c6( ) c7(T, W, c, aW, aT, v, f, af); #c1(NCAM2) c2(XP_011527884) c3(neural cell adhesion molecule 2 isoform X6) c4( ) c5(322996, 322997, 322998, 322999, 323000, 323001) c6( ) c7(T, W, c, aW, aT, v, f, af); #c1(NCAN) c2(NP_004377) c3(neurocan core protein precursor) c4(145910, 145911, 145912) c5(145913, 145914, 145915, 145916, 145917, 145918, 145919, 145920, 145921, 145922, 145923, 145924, 145925, 145926, 145927, 145928, 145929) c6( ) c7(bK, cP, fc); #c1(NCAPD2) c2(NP_055680) c3(condensin complex subunit 1) c4(182733, 182734, 182735) c5(182736, 182737, 182738, 182739, 182740, 182741, 182742, 182743, 182744, 182745, 182746, 182747, 182748, 182749, 182750, 182751, 182752, 182753) c6( ) c7(af, aT); #c1(NCAPD3) c2(NP_056076) c3(condensin-2 complex subunit D3) c4(185540, 185541, 185542) c5(185543, 185544, 185545, 185546, 185547, 185548, 185549, 185550, 185551, 185552, 185553, 185554, 185555, 185556, 185557, 185558, 185559) c6( ) c7(ae, ad); #c1(NCAPG) c2(NP_071741) c3(condensin complex subunit 3) c4(211895) c5(211896, 211897, 211898, 211899, 211900, 211901) c6( ) c7(T, cH, aW, Y, aT, ey, g); #c1(NCAPG2) c2(NP_001268861) c3(condensin-2 complex subunit G2 isoform a) c4(100237, 100238) c5(100239, 100240, 100241, 100242, 100243, 100244, 100245, 100246, 100247, 100248, 100249, 100250, 100251, 100252, 100253) c6( ) c7(aEV, bNu, bcb, ig, h, bDI, ciP); #c1(NCAPG2) c2(NP_001268862) c3(condensin-2 complex subunit G2 isoform b) c4(100254) c5(100255, 100256, 100257, 100258, 100259, 100260, 100261, 100262, 100263, 100264, 100265) c6( ) c7(aEV, bNu, bcb, ig, h, bDI, ciP); #c1(NCAPG2) c2(XP_005249604) c3(condensin-2 complex subunit G2 isoform X2) c4(269713, 269714, 269715) c5(269716, 269717, 269718, 269719, 269720, 269721, 269722, 269723, 269724, 269725, 269726, 269727, 269728) c6( ) c7(aEV, bNu, bcb, ig, h, bDI, ciP); #c1(NCAPG2) c2(XP_011514664) c3(condensin-2 complex subunit G2 isoform X5) c4( ) c5(307316, 307317, 307318, 307319, 307320) c6( ) c7(aEV, bNu, bcb, ig, h, bDI, ciP); #c1(NCBP2) c2(NP_001036005) c3(nuclear cap-binding protein subunit 2 isoform 2) c4(33671) c5(33672, 33673, 33674, 33675, 33676, 33677) c6( ) c7(aW, aT); #c1(NCBP2) c2(NP_031388) c3(nuclear cap-binding protein subunit 2 isoform 1) c4(172173, 172174) c5(172175, 172176, 172177, 172178, 172179, 172180, 172181, 172182, 172183, 172184, 172185, 172186) c6( ) c7(aW, aT); #c1(NCEH1) c2(NP_001139748) c3(neutral cholesterol ester hydrolase 1 isoform a) c4(59830) c5(59831, 59832, 59833, 59834, 59835, 59836, 59837) c6( ) c7(ea, dD, eq, ac); #c1(NCEH1) c2(NP_001139750) c3(neutral cholesterol ester hydrolase 1 isoform c) c4(59838) c5(59839, 59840, 59841, 59842, 59843) c6( ) c7(ea, dD, eq, ac); #c1(NCEH1) c2(NP_065843) c3(neutral cholesterol ester hydrolase 1 isoform b) c4(205812, 205813) c5(205814, 205815, 205816, 205817, 205818, 205819, 205820, 205821, 205822, 205823, 205824, 205825) c6( ) c7(ea, dD, eq, ac); #c1(NCF1) c2(NP_000256) c3(neutrophil cytosol factor 1) c4(5847) c5(5848, 5849, 5850, 5851, 5852, 5853) c6( ) c7(T, dA, eR, qb, e, Ju, ew, cz, jh, aj, v, C, V, cd, J, ad, ai, xE, akJ, dV, dz, ji, baJ, K, bq, asC, ae, af, et, cH, oV, Cu, zb, az, fe, iV, cil, ek, aIC, ac, AH, uR, eB, bm, wR, eg, y, ra, us); #c1(NCF1) c2(XP_011514800) c3(neutrophil cytosol factor 1 isoform X1) c4(307469) c5( ) c6( ) c7(T, dA, eR, qb, e, Ju, ew, cz, jh, aj, v, C, V, cd, J, ad, ai, xE, akJ, dV, dz, ji, baJ, K, bq, asC, ae, af, et, cH, oV, Cu, zb, az, fe, iV, cil, ek, aIC, ac, AH, uR, eB, bm, wR, eg, y, ra, us); #c1(NCF1) c2(XP_011514802) c3(neutrophil cytosol factor 1 isoform X5) c4( ) c5(307470, 307471, 307472, 307473, 307474, 307475, 307476) c6( ) c7(T, dA, eR, qb, e, Ju, ew, cz, jh, aj, v, C, V, cd, J, ad, ai, xE, akJ, dV, dz, ji, baJ, K, bq, asC, ae, af, et, cH, oV, Cu, zb, az, fe, iV, cil, ek, aIC, ac, AH, uR, eB, bm, wR, eg, y, ra, us); #c1(NCF1) c2(XP_011514803) c3(neutrophil cytosol factor 1 isoform X6) c4(307477) c5(307478, 307479, 307480, 307481, 307482) c6( ) c7(T, dA, eR, qb, e, Ju, ew, cz, jh, aj, v, C, V, cd, J, ad, ai, xE, akJ, dV, dz, ji, baJ, K, bq, asC, ae, af, et, cH, oV, Cu, zb, az, fe, iV, cil, ek, aIC, ac, AH, uR, eB, bm, wR, eg, y, ra, us); #c1(NCF2) c2(NP_001177718) c3(neutrophil cytosol factor 2 isoform 3) c4(72901) c5(72902, 72903, 72904, 72905, 72906, 72907, 72908, 72909, 72910, 72911, 72912, 72913, 72914, 72915, 72916, 72917, 72918, 72919, 72920) c6( ) c7(bM, Dw, dD, ea, iW, cz, n, v, j, eq, eA, py, aT, h, Ds, dL, ey, bq, asC, d, ciR, ciS, Cu, az, Rr, eZ, fe, dB, eB, at, y, ra, rk, cn); #c1(NCF2) c2(NP_001177723) c3(neutrophil cytosol factor 2 isoform 2) c4(72921) c5(72922, 72923, 72924, 72925, 72926, 72927) c6( ) c7(bM, Dw, dD, ea, iW, cz, n, v, j, eq, eA, py, aT, h, Ds, dL, ey, bq, asC, d, ciR, ciS, Cu, az, Rr, eZ, fe, dB, eB, at, y, ra, rk, cn); #c1(NCF2) c2(XP_005245264) c3(neutrophil cytosol factor 2 isoform X2) c4(266365, 266366) c5( ) c6( ) c7(bM, Dw, dD, ea, iW, cz, n, v, j, eq, eA, py, aT, h, Ds, dL, ey, bq, asC, d, ciR, ciS, Cu, az, Rr, eZ, fe, dB, eB, at, y, ra, rk, cn); #c1(NCF2) c2(XP_011507883) c3(neutrophil cytosol factor 2 isoform X1) c4(298095, 298096) c5(298097, 298098, 298099, 298100, 298101, 298102, 298103, 298104, 298105, 298106, 298107, 298108, 298109, 298110, 298111, 298112) c6( ) c7(bM, Dw, dD, ea, iW, cz, n, v, j, eq, eA, py, aT, h, Ds, dL, ey, bq, asC, d, ciR, ciS, Cu, az, Rr, eZ, fe, dB, eB, at, y, ra, rk, cn); #c1(NCF4) c2(NP_000622) c3(neutrophil cytosol factor 4 isoform 1) c4(14021) c5(14022, 14023, 14024, 14025, 14026, 14027) c6( ) c7(ciT, xN, dB, iW, eB, aT, gZ, r, h, fe, iV, eE, y, q, rk); #c1(NCF4) c2(NP_038202) c3(neutrophil cytosol factor 4 isoform 2) c4(176258, 176259) c5(176260, 176261, 176262, 176263, 176264, 176265, 176266, 176267, 176268, 176269, 176270, (76271, 176272) c6( ) c7(ciT, xN, dB, iW, eB, aT, gZ, r, h, fe, iV, eE, y, q, rk); #c1(NCK1) c2(NP_001177725) c3(cytoplasmic protein NCK1 isoform 2) c4(72928) c5(72929, 72930, 72931, 72932, 72933, 72934) c6( ) c7(T, dG, dy, K, ah, v, wt, BG, B, cp, sj); #c1(NCK1) c2(NP_001278928) c3(cytoplasmic protein NCK1 isoform 1) c4(111019, 111020, 111021, 111022) c5(111023, 111024, 111025, 111026, 111027, 111028, 111029, 111030, 111031, 111032, 111033, 111034, 111035, 111036, 111037, 111038, 111039, 111040, 111041, 111042, 111043, 111044, 111045, 111046, 111047, 111048, 111049, 111050) c6( ) c7(T, dG, dy, K, ah, v, wt, BG, B, cp, sj); #c1(NCK2) c2(NP_001004722) c3(cytoplasmic protein NCK2 isoform B) c4(20964) c5(20965, 20966, 20967, 20968, 20969, 20970, 20971, 20972) c6( ) c7(HQ, cZ, T, Y, ah, K, wt, dd, dw, cp, sj); #c1(NCK2) c2(NP_003572) c3(cytoplasmic protein NCK2 isoform A) c4(139249, 139250, 139251) c5(139252, 139253, 139254, 139255, 139256, 139257, 139258, 139259, 139260, 139261, 139262, 139263, 139264, 139265, 139266, 139267, 139268, 139269, 139270, 139271, 139272, 139273) c6( ) c7(HQ, cZ, T, Y, ah, K, wt, dd, dw, cp, sj); #c1(NCK2) c2(XP_011510294) c3(cytoplasmic protein NCK2 isoform X2) c4( ) c5(300877, 300878) c6( ) c7(HQ, cZ, T, Y, ah, K, wt, dd, dw, cp, sj); #c1(NCKAP1) c2(NP_038464) c3(nck-associated protein 1 isoform 1) c4(176549) c5(176550, 176551, 176552, 176553, 176554, 176555) c6( ) c7(cr, n, tv, tu, C, af, amL); #c1(NCKAP1) c2(NP_995314) c3(nck-associated protein 1 isoform 2) c4(263789) c5(263790, 263791, 263792, 263793, 263794, 263795) c6( ) c7(cr, n, tv, tu, C, af, amL); #c1(NCKAP1L) c2(NP_001171905) c3(nck-associated protein 1-like isoform 2) c4( ) c5(72007, 72008, 72009, 72010, 72011) c6( ) c7(ey, cj); #c1(NCKAP1L) c2(NP_005328) c3(nck-associated protein 1-like isoform 1) c4(155027, 155028) c5(155029, 155030, 155031, 155032, 155033, 155034, 155035) c6( ) c7(ey, cj); #c1(NCKAP5) c2(NP_997246) c3(nck-associated protein 5 isoform 1) c4(265300) c5(265301, 265302, 265303, 265304, 265305, 265306, 265307, 265308, 265309, 265310) c6( ) c7(pZ, oe, cz, gf, bk, he, V); #c1(NCKAP5) c2(NP_997364) c3(nck-associated protein 5 isoform 2) c4(265443, 265444) c5(265445, 265446, 265447, 265448, 265449, 265450, 265451, 265452, 265453, 265454, 265455, 265456, 265457, 265458, 265459, 265460, 265461, 265462, 265463, 265464, 265465, 265466, 265467, 265468, 265469, 265470, 265471, 265472) c6( ) c7(pZ, oe, cz, gf, bK, he, V); #c1(NCKAP5) c2(XP_005263717) c3(nck-associated protein 5 isoform X1) c4(278510, 278511) c5( ) c6( ) c7(pZ, oe, cz, gf, bK, he, V); #c1(NCK1PSD) c2(NP_057537) c3(NCK-interacting protein with SH3 domain isoform 1) c4(191300, 191301) c5(191302, 191303, 191304, 191305, 191306, 191307, 191308, 191309, 191310, 191311, 191312, 191313) c6( ) c7(dA, hE, Hn, er, Ne, h, By, Nf, K, eG, je, byn, Y, Ni, pR, rs, ck, iM, rq, Nk, y, jr); #c1(NCK1PSD) c2(NP_909119) c3(NCK-interacting protein with SH3 domain isoform 2) c4(258212) c5(258213, 258214, 258215, 258216, 258217, 258218) c6( ) c7(dA, hE, Hn, er, Ne, h, By, Nf, K, eG, je, byn, Y, Ni, pR, rs, ck, iM, rq, Nk, y, jr); #c1(NCL) c2(NP_005372) c3(nucleolin) c4(155524, 155525) c5(155526, 155527, 155528, 155529, 155530, 155531, 155532, 155533, 155534, 155535, 155536, 155537, 155538, 155539, 155540, 155541, 155542, 155543) c6( ) c7(Dw, T, fM, aPR, if, aA, j, ci, nA, qN, fr, AO, mC, aR, eA, eW, iC, b, py, aPQ, Bc, aT, cU, h, K, cY, aDX, adt, d, aW, Y, ff, az, eZ, aF, en, g, as, bz, rY, ck, apM, nM, eM, bE, AH, aPS, cj, U, np, aPT, FL, i, rA, B); #c1(NCOA1) c2(XP_005264682) c3(nuclear receptor coactivator 1 isoform X1) c4(279160) c5(279161, 279162, 279163, 279164, 279165, 279166, 279167, 279168, 279169, 279170) c6( ) c7(T, n, v, ci, zP, ad, afb, dP, bv, aT, ae, Or, aW, Y, ff, aq, en, as, cj, m, gT, B); #c1(NCOA1) c2(XP_005264683) c3(nuclear receptor coactivator 1 isoform X2) c4(279171) c5(279172, 279173, 279174, 279175, 279176, 279177, 279178, 279179, 279180) c6( ) c7(T, n, v, ci, zP, ad, afb, dP, bv, aT, ae, Or, aW, Y, ff, aq, en, as, cj, m, gT, B); #c1(NCOA1) c2(XP_005264685) c3(nuclear receptor coactivator 1 isoform X3) c4(279181) c5(279182, 279183, 279184, 279185, 279186, 279187, 279188, 279189, 279190, 279191, 279192) c6( ) c7(T, n, v, ci, zP, ad, afb, dP, bv, aT, ae, Or, aW, Y, ff, aq, en, as, cj, m, gT, B); #c1(NCOA2) c2(XP_005251188) c3 (nuclear receptor coactivator 2 isoform X1) c4(270845) c5(270846, 270847, 270848, 270849, 270850, 270851, 270852, 270853, 270854, 270855, 270856) c6( ) c7(gx, T, jv, kh, oA, vr, n, ci, hF, ad, aEQ, bv, Bj, aT, PD, bwV, ae, hi, gj, Or, aW, gE, cl, ck, eM, hL, apy, Xu, gT, bt, B); #c1(NCOA2) c2(XP_005251190) c3(nuclear receptor coactivator 2 isoform X2) c4(270857, 270858) c5(270859, 270860, 270861, 270862, 270863, 270864) c6( ) c7(gx, T, jv, kh, oA, vr, n, ci, hF, ad, aEQ, bv, Bj, aT, PD, bwV, ae, hi, gj, Qr, aW, gE, cl, ck, eM, hL, apy, Xu, gT, bt, B); #c1(NCOA2) c2(XP_011515743) c3(nuclear receptor coactivator 2 isoform X4) c4( ) c5(308552) c6( ) c7(gx, T, jv, kh, oA, vr, n, ci, hF, ad, aEQ, bv, Bj, aT, PD, bwV, ae, hi, gj, Ar, aW, gE, cl, ck, eM, hL, apy, Xu, gT, bt, B); #c1(NCOA3) c2(NP_001167559) c3(nuclear receptor coactivator 3 isoform d) c4(69230, 69231) c5(69232, 69233, 69234, 69235, 69236, 69237, 69238, 69239, 69240, 69241, 69242, 69243, 69244) c6( ) c7(T, aC, iW, bGr, aA, bB, n, v, oH, ci, AO, iJ, q, ad, eJ, iu, aE, Ma, aT, dL, K, ae, eL, uQ, s, iU, PE, Y, aW, eH, fc, ff, r, aF, en, cl, g, LV, t, bz, as, eF, ck, ar, Dy, eM, bE, hL, at, np, m, k, cP, ei, gT, B); #c1(NCOA3) c2(NP_006525) c3(nuclear receptor coactivator 3 isoform b) c4(165992) c5(165993, 165994, 165995, 165996, 165997, 165998, 165999, 166000, 166001, 166002, 166003, 166004, 166005, 166006, 166007, 166008) c6( ) c7(T, aC, iW, bGr, aA, bB, n, v, oH, ci, AO, iJ, q, ad, eJ, iu, aE, Ma, aT, dL, K, ae, eL, uQ, s, iU, PE, Y, aW, eH, fc, ff, r, aF, en, cl, g, LV, t, bz, as, eF, ck, ar, Dy, eM, bE, hl, at, np, m, k, cP, ei, gT, B); #c1(NCOA3) c2(NP_858045) c3(nuclear receptor coactivator 3 isoform a) c4(255304, 255305) c5(255306, 255307, 255308, 255309, 255310, 255311) c6( ) c7(T, aC, iW, bGr, aA, bB, n, v, oH, ci, AO, iJ, q, ad, eJ, iu, aE, Ma, aT, dL, K, ae, eL, uQ, s, iU, PE, Y, aW, eH, fc, ff, r, aF, en, cl, g, LV, t, bz, as, eF, ck, ar, Dy, eM, bE, hL, at, np, m, k, cP, ei, gT, B); #c1(NCOA4) c2(NP_001138732) c3 (nuclear receptor coactivator 4 isoform 1) c4(57470) c5(57471, 57472, 57473, 57474, 57475, 57476) c6( ) c7(BI, T, hd, xy, nM, nB, auz, aW, qt, Db, AS, aT, hz, ciU, Fx, ci, m, mo, t, ae, ad); #c1(NCOA4) c2(NP_001138733) c3(nuclear receptor coactivator 4 isoform 2) c4(57477, 57478, 57479, 57480, 57481, 57482, 57483) c5(57484, 57485, 57486, 57487, 57488, 57489, 57490, 57491, 57492, 57493, 57494) c6( ) c7(BI, T, hd, xy, nM, nB, auz, aW, qt, Db, AS, aT, hz, ciU, Fx, ci, m, mo, t, ae, ad); #c1(NCOA4) c2(NP_005428) c3(nuclear receptor coactivator 4 isoform 3) c4( ) c5(156047, 156048, 156049, 156050, 156051, 156052, 156053, 156054, 156055, 156056, 156057, 156058, 156059, 156060, 156061, 156062, 156063, 156064, 156065, 156066, 156067, 156068, 156069, 156070, 156071, 156072, 156073, 156074, 156075, 156076, 156077, 156078, 156079, 156080, 156081, 156082, 156083, 156084, 156085, 156086, 156087, 156088, 156089, 156090, 156091, 156092, 156093, 156094) c6( ) c7(BI, T, hd, xy, nM, nB, auz, aW, qt, Db, AS, aT, hz, ciU, Fx, ci, m, mo, t, ae, ad); #c1(NCOA5) c2(NP_066018) c3(nuclear receptor coactivator 5) c4(206751) c5( ) c6( ) c7(zw, xE, hA, y, fb, ai); #c1(NCOA5) c2(XP_005260531) c3(nuclear receptor coactivator 5 isoform X1) c4(276600) c5(276601, 276602, 276603, 276604, 276605, 276606) c6( ) c7(zw, xE, hA, y, fb, ai); #c1(NCOA5) c2(XP_011527253) c3(nuclear receptor coactivator 5 isoform X2) c4( ) c5(321857, 321858, 321859, 321860, 321861, 321862) c6( ) c7(zw, xE, hA, y, fb, ai); #c1(NCOA6) c2(NP_001229468) c3(nuclear receptor coactivator 6 isoform 2) c4( ) c5(84564, 84565, 84566, 84567, 84568) c6( ) c7(bL, T, al, aj, fr, nA, Eh, er, ad, dP, ft, aet, aT, h, K, he, af, aW, Y, ciV, aq, as, gk, kq, B); #c1(NCOA6) c2(NP_054790) c3(nuclear receptor coactivator 6 isoform 1) c4(177975) c5(177976, 177977, 177978, 177979, 177980, 177981, 177982, 177983, 177984, 177985) c6( ) c7(bL, T, al, aj, fr, nA, Eh, er, ad, dP, ft, aet, al, h, K, he, af, aW, Y, ciV, aq, as, gk, kq, B); #c1(NCOA6) c2(XP_011527031) c3(nuclear receptor coactivator 6 isoform X7) c4(321357) c5(321358) c6( ) c7(bL, T, al, aj, fr, nA, Eh, er, ad, dP, ft, aet, aT, h, K, he, af, aW, Y, ciV, aq, as, gk, kq, B); #c1(NCOA7) c2(NP_001116314) c3(nuclear receptor coactivator 7 isoform 2) c4(43760, 43761, 43762, 43763, 43764, 43765) c5(43766, 43767, 43768, 43769, 43770, 43771, 43772, 43773, 43774, 43775, 43776, 43777, 43778, 43779, 43780, 43781, 43782, 43783, 43784, 43785, 43786, 43787, 43788, 43789, 43790, 43791) c6( ) c7(cm, aW, cd, aT); #c1(NCOA7) c2(NP_001186550) c3(nuclear receptor coactivator 7 isoform 3) c4( ) c5(77776, 77777, 77778, 77779, 77780, 77781, 77782, 77783, 77784, 77785, 77786, 77787, 77788, 77789, 77790, 77791, 77792, 77793, 77794, 77795, 77796) c6( ) c7(cm, aW, cd, aT); #c1(NCOA7) c2(NP_001186551) c3(nuclear receptor coactivator 7 isoform 4) c4(77797, 77798, 77799) c5(77800, 77801, 77802, 77803, 77804, 77805, 77806, 77807, 77808, 77809, 77810, 77811, 77812, 77813, 77814, 77815, 77816, 77817) c6( ) c7(cm, aW, cd, aT); #c1(NCOA7) c2(XP_011533757) c3(nuclear receptor coactivator 7 isoform X1) c4(329906) c5(329907, 329908, 329909, 329910, 329911, 329912, 329913, 329914, 329915, 329916, 329917) c6( ) c7(cm, aW, cd, aT); #c1(NCOR1) c2(NP_001177367) c3(nuclear receptor corepressor 1 isoform 2) c4(72695) c5(72696, 72697, 72698, 72699, 72700, 72701) c6( ) c7(T, id, aZ, aTS, hF, AO, q, ad, afb, dP, x, Bj, aeX, aT, dL, K, uV, jj, ae, eL, pi, kl, d, aW, r, op, ck, vA, m, gT, kS); #c1(NCOR1) c2(NP_001177369) c3(nuclear receptor corepressor 1 isoform 3) c4(72702) c5(72703, 72704, 72705, 72706, 72707, 72708) c6( ) c7(T, id, aZ, aTS, hF, AO, q, ad, afb, dP, x, Bj, aeX, aT, dL, K, uV, jj, ae, eL, pi, kl, d, aW, r, op, ck, vA, m, gT, kS); #c1(NCOR1) c2(NP_006302) c3(nuclear receptor corepressor 1 isoform 1) c4(163834, 163835) c5(163836, 163837, 163838, 163839, 163840, 163841) c6( ) c7(T, id, aZ, aTS, hF, AO, q, ad, afb, dP, x, Bj, aeX, aT, dL, K, uV, jj, ae, eL, pi, kl, d, aW, r, op, ck, vA, m, gT, kS); #c1(NCOR1) c2(XP_005256928) c3(nuclear receptor corepressor 1 isoform X5) c4(274602) c5(274603, 274604, 274605, 274606, 274607, 274608) c6( ) c7(T, id, aZ, aTS, hF, AO, q, ad, afb, dP, x, Bj, aeX, aT, dL, K, uV, jj, ae, eL, pi, kl, d, aW, r, op, ck, vA, m, gT, kS); #c1(NCOR1) c2(XP_005256931) c3(nuclear receptor corepressor 1 isoform X9) c4( ) c5(274609) c6( ) c7(T, id, aZ, aTS, hF, AO, q, ad, afb, dP, x, Bj, aeX, aT, dL, K, uV, jj, ae, eL, pi, kl, d, aW, r, op, ck, vA, m, gT, kS); #c1(NCOR1) c2(XP_005256932) c3(nuclear receptor corepressor 1 isoform X11) c4(274610, 274611) c5( ) c6( ) c7(T, id, aZ, aTS, hF, AO, q, ad, afb, dP, x, Bj, aeX, aT, dL, K, uV, jj, ae, eL, pi, kl, d, aW, r, op, ck, vA, m, gT, kS); #c1(NCOR1) c2(XP_006721665) c3(nuclear receptor corepressor 1 isoform X3) c4(294216, 294217) c5(294218, 294219, 294220, 294221, 294222, 294223, 294224, 294225, 294226, 294227, 294228) c6( ) c7(T, id, aZ, aTS, hF, AO, q, ad, afb, dP, x, Bj, aeX, aT, dL, K, uV, jj, ae, eL, pi, kl, d, aW, r, op, ck, vA, m, gT, kS); #c1(NCOR1) c2(XP_006721667) c3(nuclear receptor corepressor 1 isoform X10) c4(294229) c5(294230, 294231, 294232, 294233, 294234, 294235, 294236, 294237, 294238, 294239, 294240, 294241) c6( ) c7(T, id, aZ, aTS, hF, AO, q, ad, afb, dP, x, Bj, aeX, aT, dL, K, uV, jj, ae, eL, pi, kl, d, aW, r, op, ck, vA, m, gT, kS); #c1(NCOR1) c2(XP_011522385) c3(nuclear receptor corepressor 1 isoform X2) c4( ) c5(317070, 317071, 317072, 317073, 317074, 317075, 317076, 317077, 317078, 317079, 317080, 317081) c6( ) c7(T, id, aZ, aTS, hF, AO, q, ad, afb, dP, x, Bj, aeX, aT, dL, K, uV, jj, ae, eL, pi, kl, d, aW, r, op, ck, vA, m, gT, kS); #c1(NCOR2) c2(NP_001070729) c3(nuclear receptor corepressor 2 isoform 2) c4(34801, 34802) c5(34803, 34804, 34805, 34806, 34807, 34808, 34809, 34810, 34811, 34812, 34813, 34814) c6( ) c7(bM, T, fM, aA, bB, fd, v, ad, aT, h, Dj, bK, qM, jj, ae, Dg, aW, aF, cm, op, Qi, ac, bE, vA, pg, bC, gT, kS); #c1(NCOR2) c2(NP_001193583) c3(nuclear receptor corepressor 2 isoform 3) c4(82282, 82283, 82284) c5(82285, 82286, 82287, 82288, 82289, 82290, 82291, 82292, 82293, 82294, 82295, 82296, 82297, 82298, 82299, 82300, 82301, 82302, 82303, 82304, 82305, 82306, 82307, 82308) c6( ) c7(bM, T, fM, aA, bB, fd, v, ad, aT, h, Dj, bK, qM, jj, ae, Dg, aW, aF, cm, op, Qi, ac, bE, vA, pg, bC, gT, KS); #c1(NCOR2) c2(NP_006303) c3(nuclear receptor corepressor 2 isoform 1) c4(163842, 163843, 163844) c5(163845, 163846, 163847, 163848, 163849, 163850, 163851, 163852, 163853, 163854, 163855, 163856) c6( ) c7(bM, T, fM, aA, bB, fd, v, ad, aT, h, Dj, bK, qM, jj, ae, Dg, aW, aF, cm, op, Qi, ac, bE, vA, pg, bC, gT, kS); #c1(NCR1) c2(NP_001138929) c3(natural cytotoxicity triggering receptor 1 isoform b precursor) c4(58244) c5(58245, 58246, 58247, 58248, 58249, 58250, 58251, 58252, 58253, 58254, 58255, 58256, 58257, 58258, 58259, 58260, 58261, 58262, 58263, 58264, 58265, 58266, 58267, 58268, 58269, 58270, 58271, 58272, 58273, 58274) c6( ) c7(ck, eW, A, d, JZ, iU, Y, ds, jx, iK, bt, fd, h, uz, eo, K, as, zM, V, eA); #c1(NCR1) c2(NP_001138930) c3(natural cytotoxicity triggering receptor 1 isoform c precursor) c4(58275) c5(58276, 58277, 58278, 58279, 58280, 58281) c6( ) c7(ck, eW, A, d, JZ, iU, Y, ds, jx, iK, bt, fd, h, uz, eo, K, as, zM, V, eA); #c1(NCR1) c2(NP_001229285) c3(natural cytotoxicity triggering receptor 1 isoform d precursor) c4(84151, 84152) c5(84153, 84154, 84155, 84156, 84157, 84158, 84159, 84160, 84161, 84162, 84163, 84164) c6( ) c7(ck, eW, A, d, JZ, iU, Y, ds, jx, iK, bt, fd, h, uz, eo, K, as, zM, V, eA); #c1(NCR1) c2(NP_004820) c3(natural cytotoxicity triggering receptor 1 isoform a precursor) c4(150047) c5(150048, 150049, 150050, 150051, 150052, 150053) c6( ) c7(ck, eW, A, d, JZ, iU, Y, ds, jx, iK, bt, fd, h, uz, eo, K, as, zM, V, eA); #c1(NCR2) c2(NP_001186438) c3(natural cytotoxicity triggering receptor 2 isoform 2 precursor) c4(77640) c5(77641, 77642, 77643, 77644, 77645, 77646) c6( ) c7(ck, mR, ac, Y, ds, eZ, fd, uz, azm, j, ci, bt, af); #c1(NCR2) c2(NP_001186439) c3(natural cytotoxicity triggering receptor 2 isoform 3 precursor) c4(77647) c5(77648, 77649, 77650, 77651, 77652, 77653) c6( ) c7(ck, mR, ac, Y, ds, eZ, fd, uz, azm, j, ci, bt, af); #c1(NCR2) c2(NP_004819) c3(natural cytotoxicity triggering receptor 2 isoform 1 precursor) c4(150040) c5(150041, 150042, 150043, 150044, 150045, 150046) c6( ) c7(ck, mR, ac, Y, ds, eZ, fd, uz, azm, j, ci, bt, af); #c1(NCR3) c2(NP_001138938) c3(natural cytotoxicity triggering receptor 3 isoform b precursor) c4(58349) c5(58350, 58351, 58352, 58353, 58354, 58355, 58356, 58357, 58358, 58359, 58360, 58361, 58362, 58363, 58364, 58365, 58366, 58367, 58368, 58369, 58370, 58371, 58372, 58373, 58374, 58375, 58376, 58377) c6( ) c7(ciW, zJ, T, ck, d, A, iU, dV, eZ, h, fd, Hs, v, jh, eg, hH, bt, fb, vk); #c1(NCR3) c2(NP_001138939) c3(natural cytotoxicity triggering receptor 3 isoform c precursor) c4(58378, 58379) c5(58380, 58381, 58382, 58383, 58384, 58385, 58386, 58387, 58388, 58389, 58390, 58391, 58392, 58393, 58394, 58395, 58396, 58397, 58398, 58399, 58400, 58401, 58402, 58403, 58404, 58405, 58406, 58407, 58408, 58409, 58410, 58411, 58412, 58413, 58414, 58415, 58416, 58417, 58418, 58419, 58420, 58421, 58422, 58423, 58424, 58425, 58426, 58427, 58428, 58429, 58430, 58431, 58432, 58433, 58434, 58435) c6( ) c7(ciW, zJ, T, ck, d, A, iU, dV, eZ, h, fd, Hs, v, jh, eg, hH, bt, fb, vk); #c1(NCR3) c2(XP_006715112) c3(natural cytotoxicity triggering receptor 3 isoform X8) c4(288705) c5(288706, 288707, 288708, 288709, 288710, 288711, 288712, 288713, 288714, 288715, 288716, 288717, 288718, 288719, 288720, 288721, 288722, 288723, 288724, 288725, 288726, 288727, 288728, 288729, 288730, 288731, 288732, 288733) c6( ) c7(ciW, zJ, T, ck, d, A, iU, dV, eZ, h, fd, Hs, v, jh, eg, hH, bt, fb, vk); #c1(NCR3LG1) c2(NP_001189368) c3(natural cytotoxicity triggering receptor 3 ligand 1 precursor) c4(79294) c5(79295, 79296, 79297, 79298, 79299, 79300, 79301, 79302, 79303, 79304) c6( ) c7(T, jh, Y, C); #c1(NCS1) c2(NP_001122298) c3(neuronal calcium sensor 1 isoform 2) c4(46458) c5(46459, 46460, 46461, 46462, 46463, 46464) c6( ) c7(nf, aNU, bK, cm, cd, bN); #c1(NCS1) c2(NP_055101) c3(neuronal calcium sensor 1 isoform 1) c4(178948) c5(178949, 178950, 178951, 178952, 178953, 178954, 178955) c6( ) c7(nf, aNU, bk, cm, cd, bN); #c1(NCSTN) c2(NP_001277113) c3(nicastrin isoform 2) c4(109023, 109024) c5(109025, 109026, 109027, 109028, 109029, 109030, 109031, 109032, 109033, 109034) c6( ) c7(ciX, T, af, bbx, aaH, aW, aT, cm, fK, L); #c1(NCSTN) c2(NP_001277115) c3(nicastrin isoform 3 precursor) c4(109035, 109036) c5(109037, 109038, 109039, 109040, 109041, 109042, 109043, 109044, 109045, 109046, 109047, 109048) c6( ) c7(ciX, T, af, bbx, aaH, aW, aT, cm, fK, L); #c1(NCSTN) c2(NP_056146) c3(nicastrin isoform 1 precursor) c4(185952, 185953, 185954) c5(185955, 185956, 185957, 185958, 185959, 185960, 185961) c6( ) c7(ciX, T, af, bbx, aaH, aW, aT, cm, fK, L); #c1(NCSTN) c2(XP_005245110) c3(nicastrin isoform X3) c4( ) c5(266289, 266290, 266291, 266292, 266293, 266294, 266295, 266296, 266297, 266298, 266299, 266300) c6( ) c7(ciX, T, af, bbx, aaH, aW, aT, cm, fK, L); #c1(NCSTN) c2(XP_011507665) c3(nicastrin isoform XI) c4(297621, 297622, 297623) c5( ) c6( ) c7(ciX, T, af, bbx, aaH, aW, aT, cm, fK, L); #c1(NCSTN) c2(XP_011507666) c3(nicastrin isoform X2) c4(297624) c5(297625, 297626, 297627, 297628, 297629, 297630, 297631, 297632, 297633, 297634, 297635, 297636, 297637, 297638, 297639, 297640, 297641, 297642, 297643, 297644, 297645, 297646, 297647, 297648, 297649, 297650, 297651, 297652, 297653) c6( ) c7(ciX, T, af, bbx, aaH, aW, aT, cm, fK, L); #c1(NDC80) c2(NP_006092) c3 (kinetochore protein NDC80 homolog) c4(161831, 161832) c5(161833, 161834, 161835, 161836, 161837, 161838, 161839, 161840, 161841, 161842, 161843, 161844) c6( ) c7(bF, T, eM, NE, AM, aW, zX, nO, hL, aT, aA, apx, h, af, ey, m, bU, bz, eW, cl); #c1(NDE1) c2(NP_060138) c3(nuclear distribution protein nudE homolog 1) c4(194355) c5(194356, 194357, 194358, 194359, 194360, 194361, 194362, 194363, 194364, 194365, 194366, 194367) c6( ) c7(ciZ, FB, FD, r, gl, cja, q, m, QK, ciY, gD, aR); #c1(NDE1) c2(XP_006720961) c3(nuclear distribution protein nudE homolog 1 isoform X2) c4(293793) c5(293794, 293795, 293796, 293797, 293798, 293799, 293800, 293801) c6( ) c7(ciZ, FB, FD, r, gl, cja, q, m, QK, ciY, gD, aR); #c1(NDE1) c2(XP_011520855) c3(nuclear distribution protein nudE homolog 1 isoform X7) c4( ) c5(315838) c6( ) c7(ciZ, FB, FD, r, gl, cja, q, m, QK, ciY, gD, aR); #c1(NDEL1) c2(NP_001020750) c3(nuclear distribution protein nudE-like 1 isoform A) c4(28976) c5(28977, 28978, 28979, 28980, 28981, 28982, 28983, 28984, 28985, 28986, 28987, 28988) c6( ) c7(bJL, qx, cL, oQ, cm, ai, o, bN); #c1(NDEL1) c2(NP_110435) c3(nuclear distribution protein nudE-like 1 isoform B) c4(220354, 220355) c5(220356, 220357, 220358, 220359, 220360, 220361, 220362, 220363, 220364, 220365, 220366, 220367) c6( ) c7(bJL, qx, cL, od, cm, ai, o, bN); #c1(NDEL1) c2(XP_006721643) c3(nuclear distribution protein nudE-like 1 isoform X2) c4(294139) c5( ) c6( ) c7(bJL, qx, cL, ol, cm, ai, o, bN); #c1(NDFIP1) c2(NP_085048) c3(NEDD4 family-interacting protein 1) c4(219207, 219208) c5(219209, 219210, 219211, 219212, 219213, 219214, 219215, 219216, 219217, 219218, 219219, 219220) c6( ) c7(bM, V, ac, bu); #c1(NDFIP2) c2(NP_061953) c3(NEDD4 family-interacting protein 2 isoform 1) c4(201286) c5(201287, 201288, 201289, 201290, 201291, 201292) c6( ) c7(bL, aC, Nc); #c1(NDN) c2(NP_002478) c3(necdin) c4(129171) c5(129172, 129173, 129174, 129175, 129176, 129177) c6( ) c7(T, id, oe, amj, v, ci, rc, yn, hb, aT, K, he, zQ, d, aW, Y, Tp, t, cm, pb, bN, Qi, eD, agl, m, UF); #c1(NDNF) c2(NP_078850) c3(protein NDNF precursor) c4(215719, 215720, 215721) c5(215722, 215723, 215724, 215725, 215726, 215727, 215728, 215729, 215730, 215731, 215732, 215733, 215734, 215735, 215736, 215737, 215738, 215739, 215740, 215741, 215742, 215743, 215744, 215745) c6(215746, 215747, 215748, 215749, 215750, 215751) c7(dP, cy, aS); #c1(NDP) c2(NP_000257) c3(norrin precursor) c4(5854) c5(5855, 5856, 5857, 5858, 5859, 5860) c6( ) c7(Ck, lR, byj, af, aW, ajw, aT, cjb, vt, B, cC); #c1(NDRG1) c2(NP_001128714) c3(protein NDRG1 isoform 1) c4(50579, 50580, 50581) c5(50582, 50583, 50584, 50585, 50586, 50587, 50588, 50589, 50590, 50591, 50592, 50593, 50594, 50595, 50596, 50597, 50598, 50599, 50600, 50601, 50602, 50603, 50604, 50605, 50606, 50607, 50608, 50609, 50610, 50611, 50612, 50613, 50614) c6( ) c7(T, aC, if, agU, aA, bB, n, fr, ci, bX, qN, mF, V, q, ad, eA, Zp, av, ft, cjc, RI, aE, aT, yu, dL, K, j, bU, hu, ae, d, B, s, aW, Y, PX, ff, r, aq, kp, aF, en, cl, g, t, as, bz, cm, es, ok, ql, eM, iA, dB, bE, hL, Rm, np, m, bt, it, X, BA); #c1(NDRG1) c2(NP_001245361) c3(protein NDRG1 isoform 2) c4( ) c5(93252) c6( ) c7(T, aC, if, agU, aA, bB, n, fr, ci, bX, qN, mF, V, q, ad, eA, Zp, av, ft, cjc, RI, aE, aT, yu, dL, K, j, bU, hu, ae, d, B, s, aW, Y, PX, ff, r, aq, kp, aF, en, cl, g, t, as, bz, cm, es, ok, ql, eM, iA, dB, bE, hL, Rm, np, m, bt, it, X, BA); #c1(NDRG1) c2(NP_001245362) c3(protein NDRG1 isoform 3) c4(93253, 93254, 93255) c5(93256, 93257, 93258, 93259, 93260, 93261, 93262, 93263, 93264, 93265, 93266, 93267, 93268, 93269, 93270, 93271, 93272) c6( ) c7(T, aC, if, agU, aA, bB, n, fr, ci, bX, qN, mF, V, q, ad, eA, Zp, av, ft, cjc, RI, aE, aT, yu, dL, K, j, bU, hu, ae, d, B, s, aW, Y, PX, ff, r, aq, kp, aF, en, cl, g, t, as, bz, cm, es, ok, ql, eM, iA, dB, bE, hL, Rm, np, m, bt, it, X, BA); #c1(NDRG1) c2(XP_011515093) c3(protein NDRG1 isoform X1) c4(307953, 307954, 307955) c5(307956, 307957, 307958, 307959) c6( ) c7(T, aC, if, agU, aA, bB, n, fr, ci, bX, qN, mF, V, q, ad, eA, Zp, av, ft, cjc, RI, aE, aT, yu, dL, K, j, bU, hu, ae, d, B, s, aW, Y, PX, ff, r, aq, kp, aF, en, cl, g, t, as, bz, cm, es, ok, ql, eM, iA, dB, bE, hL, Rm, np, m, bt, it, X, BA); #c1(NDRG1) c2(XP_011515094) c3(protein NDRG1 isoform X2) c4( ) c5(307960, 307961, 307962, 307963, 307964, 307965) c6( ) c7(T, aC, if, agU, aA, bB, n, fr, ci, bX, qN, mF, V, q, ad, eA, Zp, av, ft, cjc, RI, aE, aT, yu, dL, K, j, bU, hu, ae, d, B, s, aW, Y, PX, ff, r, aq, kp, aF, en, cl, g, t, as, bz, cm, es, ok, ql, eM, iA, dB, bE, hL, Rm, np, m, bt, it, X, BA); #c1(NDRG2) c2(NP_001269140) c3 (protein NDRG2 isoform c) c4(100548, 100549, 100550, 100551, 100552, 100553, 100554, 100555, 100556, 100557, 100558, 100559, 100560, 100561, 100562, 100563, 100564, 100565) c5(100566, 100567, 100568, 100569, 100570, 100571, 100572, 100573, 100574, 100575, 100576, 100577, 100578, 100579, 100580, 100581, 100582, 100583, 100584, 100585, 100586, 100587, 100588, 100589, 100590, 100591, 100592, 100593, 100594, 100595) c6( ) c7(T, aC, if, oe, lH, qy, aA, bB, n, fr, ci, qN, q, ad, eA, ip, hd, py, aE, aT, dL, AN, K, ae, af, eL, d, B, s, je, aW, ev, r, aq, vs, aF, as, iR, iM, iA, nB, bE, go, it, X, L); #c1(NDRG2) c2(NP_001269141) c3(protein NDRG2 isoform d) c4(100596) c5(100597, 100598, 100599, 100600, 100601, 100602, 100603, 100604, 100605, 100606, 100607, 100608, 100609, 100610, 100611, 100612, 100613, 100614, 100615, 100616, 100617, 100618, 100619, 100620, 100621, 100622, 100623, 100624, 100625, 100626, 100627, 100628, 100629, 100630, 100631, 100632, 100633, 100634, 100635, 100636, 100637, 100638, 100639, 100640, 100641, 100642, 100643, 100644, 100645, 100646, 100647, 100648, 100649, 100650, 100651, 100652, 100653, 100654, 100655, 100656, 100657, 100658, 100659, 100660, 100661, 100662, 100663, 100664, 100665, 100666, 100667, 100668, 100669, 100670, 100671, 100672, 100673, 100674, 100675, 100676, 100677, 100678, 100679, 100680, 100681, 100682, 100683, 100684, 100685, 100686, 100687, 100688, 100689) c6( ) c7(T, aC, if, oe, lH, qy, aA, bB, n, fr, ci, qN, q, ad, eA, ip, hd, py, aE, aT, dL, AN, K, ae, af, eL, d, B, s, je, aW, ev, r, aq, vs, aF, as, iR, iM, iA, nB, bE, go, it, X, L); #c1(NDRG2) c2(NP_001269144) c3(protein NDRG2 isoform e) c4(100690, 100691, 100692, 100693, 100694, 100695, 100696, 100697, 100698, 100699, 100700, 100701) c5(100702, 100703, 100704, 100705, 100706, 100707) c6( ) c7(T, aC, if, oe, lH, qy, aA, bB, n, fr, ci, qN, q, ad, eA, ip, hd, py, aE, aT, dL, AN, K, ae, af, eL, d, B, s, je, aW, ev, r, aq, vs, aF, as, iR, iM, iA, nB, bE, go, it, X, L); #c1(NDRG2) c2(NP_963294) c3(protein NDRG2 isoform b) c4(262353) c5(262354, 262355, 262356, 262357, 262358, 262359, 262360, 262361, 262362, 262363, 262364, 262365, 262366, 262367, 262368, 262369, 262370, 262371, 262372, 262373, 262374, 262375, 262376, 262377, 262378) c6( ) c7(T, aC, if, oe, lH, qy, aA, bB, n, fr, ci, qN, q, ad, eA, ip, hd, py, aE, aT, dL, AN, K, ae, af, eL, d, B, s, je, aW, ev, r, aq, vs, aF, as, iR, iM, iA, nB, bE, go, it, X, L); #c1(NDRG2) c2(XP_005267968) c3(protein NDRG2 isoform X3) c4( ) c5(281334, 281335, 281336, 281337, 281338, 281339, 281340, 281341, 281342, 281343, 281344, 281345, 281346, 281347, 281348, 281349, 281350, 281351, 281352, 281353, 281354, 281355, 281356, 281357, 281358, 281359, 281360, 281361, 281362, 281363, 281364, 281365, 281366, 281367, 281368, 281369, 281370, 281371, 281372, 281373, 281374, 281375, 281376, 281377, 281378, 281379, 281380, 281381, 281382, 281383, 281384, 281385, 281386, 281387, 281388, 281389, 281390, 281391, 281392, 281393, 281394, 281395, 281396, 281397, 281398, 281399, 281400, 281401, 281402, 281403, 281404, 281405) c6( ) c7(T, aC, if, oe, lH, qy, aA, bB, n, fr, ci, qN, q, ad, eA, ip, hd, py, aE, aT, dL, AN, K, ae, af, eL, d, B, s, je, aW, ev, r, aq, vs, aF, as, iR, iM, iA, nB, bE, go, it, X, L); #c1(NDRG2) c2(XP_011535300) c3(protein NDRG2 isoform X1) c4(332311, 332312) c5(332313, 332314, 332315, 332316, 332317, 332318, 332319, 332320, 332321, 332322, 332323, 332324, 332325, 332326, 332327, 332328, 332329, 332330, 332331, 332332, 332333, 332334) c6( ) c7(T, aC, if, oe, lH, qy, aA, bB, n, fr, ci, qN, q, ad, eA, ip, hd, py, aE, aT, dL, AN, K, ae, af, eL, d, B, s, je, aW, ev, r, aq, vs, aF, as, iR, iM, iA, nB, bE, go, it, X, L); #c1(NDRG3) c2(NP_071922) c3(protein NDRG3 isoform b) c4(212328, 212329) c5(212330, 212331, 212332, 212333, 212334, 212335) c6( ) c7(T, ae, ad); #c1(NDRG3) c2(NP_114402) c3(protein NDRG3 isoform a) c4(222545) c5(222546, 222547, 222548, 222549, 222550, 222551) c6( ) c7(T, ae, ad); #c1(NDRG3) c2(XP_011527228) c3(protein NDRG3 isoform X2) c4( ) c5(321779, 321780, 321781, 321782, 321783, 321784) c6( ) c7(T, ae, ad); #c1(NDRG3) c2(XP_011527230) c3(protein NDRG3 isoform X6) c4(321785) c5(321786, 321787, 321788, 321789, 321790, 321791) c6( ) c7(T, ae, ad); #c1(NDRG4) c2(NP_001123959) c3(protein NDRG4 isoform 2) c4(48139) c5(48140, 48141, 48142, 48143, 48144, 48145, 48146, 48147) c6( ) c7(q, K, ek, r); #c1(NDRG4) c2(NP_001229762) c3(protein NDRG4 isoform 4) c4(85196) c5(85197, 85198, 85199, 85200, 85201, 85202) c6( ) c7(q, K, ek, r); #c1(NDRG4) c2(NP_001229764) c3(protein NDRG4 isoform 6) c4(85203) c5(85204, 85205, 85206, 85207, 85208, 85209, 85210, 85211, 85212, 85213, 85214) c6( ) c7(q, K, ek, r); #c1(NDRG4) c2(NP_001229765) c3(protein NDRG4 isoform 3) c4(85215) c5(85216, 85217, 85218, 85219, 85220, 85221) c6( ) c7(q, K, ek, r); #c1(NDRG4) c2(NP_075061) c3(protein NDRG4 isoform 1) c4(213614) c5(213615, 213616, 213617, 213618, 213619, 213620, 213621, 213622) c6( ) c7(q, K, e, r); #c1(NDRG4) c2(XP_006721322) c3(protein NDRG4 isoform X9) c4(293939) c5( ) c6( ) c7(q, K, ek, r); #c1(NDRG4) c2(XP_006721324) c3(protein NDRG4 isoform X10) c4( ) c5(293940) c6( ) c7(q, K, ek, r); #c1(NDST1) c2(XP_005268491) c3(bifunctional heparan sulfate N-deacetylase/N-sulfotransferase 1 isoform X3) c4(281855) c5(281856, 281857, 281858, 281859, 281860) c6( ) c7(T, ac, RX, Az, Xg, aT, va, dy, j, nA, bms); #c1(NDST1) c2(XP_005268495) c3(bifunctional heparan sulfate N-deacetylase/N-sulfotransferase 1 isoform X4) c4(281861) c5( ) c6( ) c7(T, ac, RX, Az, Xg, aT, va, dy, j, nA, bms); #c1(NDST1) c2(XP_005268496) c3(bifunctional heparan sulfate N-deacetylase/N-sulfotransferase 1 isoform X2) c4(281862) c5( ) c6( ) c7(T, ac, RX, Az, Xg, al, va, dy, j, nA, bms); #c1(NDST1) c2(XP_005268498) c3(bifunctional heparan sulfate N-deacetylase/N-sulfotransferase 1 isoform X6) c4( ) c5(281863, 281864, 281865, 281866, 281867, 281868, 281869, 281870, 281871, 281872, 281873, 281874, 281875) c6( ) c7(T, ac, RX, Az, Xg, aT, va, dy, j, nA, bms); #c1(NDST1) c2(XP_006714846) c3(bifunctional heparan sulfate N-deacetylase/N-sulfotransferase 1 isoform X1) c4(288257) c5(288258, 288259, 288260, 288261, 288262, 288263) c6( ) c7(T, ac, RX, Az, Xg, aT, va, dy, j, nA, bms); #c1(NDST2) c2(XP_005270312) c3(bifunctional heparan sulfate N-deacetylase/N-sulfotransferase 2 isoform X3) c4( ) c5(283333) c6( ) c7(q, r); #c1(NDST2) c2(XP_005270313) c3(bifunctional heparan sulfate N-deacetylase/N-sulfotransferase 2 isoform X4) c4( ) c5(283334, 283335, 283336, 283337, 283338, 283339) c6( ) c7(q, r); #c1(NDST2) c2(XP_006718109) c3(bifunctional heparan sulfate N-deacetylase/N-sulfotransferase 2 isoform X2) c4(291520) c5( ) c6( ) c7(q, r); #c1(NDST2) c2(XP_011538612) c3(bifunctional heparan sulfate N-deacetylase/N-sulfotransferase 2 isoform X1) c4(336321, 336322) c5(336323, 336324, 336325, 336326, 336327, 336328, 336329, 336330, 336331, 336332, 336333, 336334) c6( ) c7(q, r); #c1(NDST3) c2(NP_004775) c3(bifunctional heparan sulfate N-deacetylase/N-sulfotransferase 3) c4(149519) c5(149520, 149521, 149522, 149523, 149524, 149525) c6( ) c7(bL, A, cz, j, bK, y); #c1(NDST3) c2(XP_011530717) c3(bifunctional heparan sulfate N-deacetylase/N-sulfotransferase 3 isoform X3) c4(326803) c5( ) c6( ) c7(bL, A, cz, j, bK, y); #c1(NDST3) c2(XP_011530718) c3(bifunctional heparan sulfate N-deacetylase/N-sulfotransferase 3 isoform X4) c4( ) c5(326804, 326805, 326806, 326807, 326808, 326809) c6( ) c7(bL, A, cz, j, bK, y); #c1(NDST4) c2(NP_072091) c3(bifunctional heparan sulfate N-deacetylase/N-sulfotransferase 4) c4(212663) c5(212664, 212665, 212666, 212667, 212668, 212669) c6( ) c7(T, q, j, VT, r); #c1(NDUFA1) c2(NP_004532) c3(NADH dehydrogenase [ubiquinone] 1 alpha subcomplex subunit 1) c4(147346) c5(147347, 147348, 147349, 147350, 147351, 147352) c6( ) c7(yr, sS, bBz, awN, cjd, aM, afQ, U, bxk, aNG, vd, eL, an, px, kC, ajU); #c1(NDUFA10) c2(NP_004535) c3(NADH dehydrogenase [ubiquinone] 1 alpha subcomplex subunit 10, mitochondrial precursor) c4(147369, 147370) c5(147371, 147372, 147373, 147374, 147375, 147376, 147377, 147378, 147379, 147380, 147381, 147382) c6( ) c7(aM, bxl, ad, aju); #c1(NDUFA11) c2(NP_001180304) c3(NADH dehydrogenase [ubiquinone] 1 alpha subcomplex subunit 11 isoform 2) c4(73783) c5(73784, 73785, 73786, 73787, 73788, 73789) c6( ) c7(bxk, aM); #c1(NDUFA11) c2(NP_783313) c3(NADH dehydrogenase [ubiquinone] I alpha subcomplex subunit Il isoform 1) c4(251602) c5(251603, 251604, 251605, 251606, 251607, 251608) c6( ) c7(bxk, aM); #c1(NDUFA12) c2(NP_001245267) c3(NADH dehydrogenase [ubiquinone] 1 alpha subcomplex subunit (2 isoform b) c4(92964) c5(92965, 92966, 92967, 92968, 92969) c6( ) c7(aM, bxl, ajU); #c1(NDUFA12) c2(NP_061326) c3(NADH dehydrogenase [ubiquinone] 1 alpha subcomplex subunit 12 isoform a) c4(200028) c5(200029, 200030, 200031, 200032, 200033, 200034, 200035) c6( ) c7(aM, bxl, ajU); #c1(NDUFA13) c2(NP_057049) c3(NADH dehydrogenase [ubiquinone] 1 alpha subcomplex subunit 13) c4(188620, 188621) c5(188622, 188623, 188624, 188625, 188626, 188627, 188628, 188629, 188630, 188631, 188632, 188633) c6( ) c7(T, if, aA, fr, ci, eA, ad, Db, cje, aT, h, ae, eu, d, B, aW, aF, es, iA, nB, X, iu); #c1(NDUFA2) c2(NP_001171941) c3(NADH dehydrogenase [ubiquinone] 1 alpha subcomplex subunit 2 isoform 2) c4(72060) c5(72061, 72062, 72063, 72064, 72065, 72066) c6( ) c7(oO, bM, T, JN, Js, aFg, ea, hV, e, qx, al, fd, cL, Vd, Jx, Qp, fw, aDI, Tz, AW, xE, aE, rw, Gb, dV, K, dQ, aFk, dF, eL, hm, yo, aAg, aW, az, hl, hJ, tU, eE, bw, ba, eM, JA, dc, ki, cj, afp, Dt, eg, Le, Wx, dD, zt, aw, n, fr, jG, pM, aCQ, Tl, zo, ai, eW, ec, aFq, cU, h, dL, aFi, wb, ey, aDp, A, mR, sp, fe, nN, jT, aFj, Wt, asE, ar, kT, aBs, uz, aEG, eo, fL, B, Jo, bL, fM, dA, jx, aA, tc, ol, v, j, C, aFn, V, aFv, q, o, er, rS, ld, aT, yu, jj, aFm, Cy, ae, ajU, aFs, s, iU, bg, Pg, r, aFo, lo, pb, ck, cT, ac, c, ig, wD, gX, tM, mf, cS, JH, fD, Da, zu, bt, ao, Kd, aD, iW, jh, Jz, jE, ah, eq, ad, kL, hX, Jm, aFu, F, ib, aFl, aFh, Dq, Os, hD, LA, vB, NM, uQ, d, cW, qt, aFt, eZ, bxl, gE, hp, Fq, aF, aFd, iV, eK, gk, pc, zr, yE, z, aka, eB, wz, aS, ei, lf, fh, y); #c1(NDUFA2) c2(NP_002479) c3 (NADH dehydrogenase [ubiquinone] 1 alpha subcomplex subunit 2 isoform 1) c4(12918) c5(129179, 129180, 129181, 129182, 129183, 129184) c6( ) c7(oO, bM, T, JN, Js, aFg, ea, hV, e, qx, al, fd, cL, Vd, Jx, Qp, fw, aDI, Tz, AW, xE, aE, rw, Gb, dV, K, dQ, aFk, dF, eL, hm, yo, aAg, aW, az, hl, hJ, tU, eE, bw, ba, eM, JA, dc, ki, cj, afp, Dt, eg, Le, Wx, dD, zt, aw, n, fr, jG, pM, aCQ, Tl, zo, ai, eW, ec, aFq, cU, h, dL, aFi, wb, ey, aDp, A, mR, sp, fe, nN, jT, aFj, Wt, asE, ar, kT, aBs, uz, aEG, eo, fL, B, Jo, bL, fM, dA, jx, aA, tc, ol, v, j, C, aFn, V, aFv, q, o, er, rS, ld, aT, yu, jj, aFm, Cy, ae, ajU, aFs, s, iU, bg, Pg, r, aFo, lo, pb, ck, cT, ac, c, ig, wO, gX, tM, mf, cS, JH, fD, Da, zu, bt, ao, Kd, aD, iW, jh, Jz, jE, ah, eq, ad, kL, hX, Jm, aFu, F, ib, aFl, aFh, Dq, Os, hD, LA, vB, NM, uQ, d, cW, qt, aFt, eZ, bxl, gE, hp, Fq, aF, aFd, iV, ek, gk, pc, zr, yE, z, aka, eB, wz, aS, ei, lf, fh, y); #c1(NDUFA5) c2(NP_001269349) c3(NADH dehydrogenase [ubiquinone] 1 alpha subcomplex subunit 5 isoform 3) c4(101236) c5(101237, 101238, 101239, 101240, 101241, 101242) c6( ) c7(EV, cc, ad, bN, SE); #c1(NDUFA5) c2(NP_001269351) c3(NADH dehydrogenase [ubiquinone] 1 alpha subcomplex subunit 5 isoform 5) c4( ) c5(101243, 101244, 101245, 101246, 101247, 101248) c6( ) c7(EV, cc, ad, bN, SE); #c1(NDUFA5) c2(NP_004991) c3(NADH dehydrogenase [ubiquinone] 1 alpha subcomplex subunit 5 isoform 1) c4(151810, 151811) c5(151812, 151813, 151814, 151815, 151816, 151817) c6( ) c7(EV, cc, ad, bN, SE); #c1(NDUFA6) c2(NP_002481) c3(NADH dehydrogenase [ubiquinone] 1 alpha subcomplex subunit 6) c4(129185) c5(129186, 129187, 129188, 129189, 129190, 129191) c6( ) c7(h, hy, ne, BN); #c1(NDUFA9) c2(NP_004993) c3(NADH dehydrogenase [ubiquinone] 1 alpha subcomplex subunit 9, mitochondrial precursor) c4(151818) c5(151819, 151820, 151821, 151822, 151823, 151824, 151825) c6( ) c7(bxl, ad); #c1(NDUFAB1) c2(NP_004994) c3(acyl carrier protein, mitochondrial precursor) c4(151826) c5(151827, 151828, 151829, 151830, 151831, 151832, 151833, 151834) c6( ) c7(BO, cz, hR, aj, aUP, bK, j, aKr, y, zn); #c1(NDUFAF1) c2(NP_057097) c3(complex 1 intermediate-associated protein 30, mitochondrial precursor) c4(188890, 188891) c5(188892, 188893, 188894, 188895, 188896, 188897) c6( ) c7(bxk, cc, kC, aM, vc); #c1(NDUFAF1) c2(XP_011519961) c3(complex 1 intermediate-associated protein 30, mitochondrial isoform X2) c4(314786) c5(314787, 314788, 314789, 314790, 314791, 314792, 314793, 314794, 314795, 314796, 314797, 314798) c6( ) c7(bxk, cc, kC, aM, vc); #c1(NDUFAF2) c2(NP_777549) c3(mimitin, mitochondrial) c4(251122) c5(251123, 251124, 251125, 251126, 251127, 251128) c6( ) c7(hb, aM, bwo, ajU, ff, bxk, bxl, K, en, lM, kC, it, ad); #c1(NDUFAF3) c2(NP_951032) c3(NADH dehydrogenase [ubiquinone] 1 alpha subcomplex assembly factor 3 isoform a precursor) c4(260986, 260987) c5(260988, 260989, 260990, 260991, 260992, 260993, 260994, 260995, 260996) c6( ) c7(bxk, aW, kC, aM, aT); #c1(NDUFAF3) c2(NP_951033) c3(NADH dehydrogenase [ubiquinone] 1 alpha subcomplex assembly factor 3 isoform b) c4( ) c5(260997, 260998, 260999, 261000, 261001, 261002, 261003, 261004, 261005, 261006, 261007) c6( ) c7(bxk, aW, kC, aM, aT); #c1(NDUFAF4) c2(NP_054884) c3(NADH dehydrogenase [ubiquinone] 1 alpha subcomplex assembly factor 4) c4(178183) c5(178184, 178185, 178186, 178187, 178188, 178189) c6( ) c7(aW, aM, jh, aT, bxk, px, kC); #c1(NDUFAF5) c2(NP_001034464) c3(NADH dehydrogenase [ubiquinone] 1 alpha subcomplex assembly factor 5 isoform 2) c4(31746) c5(31747, 31748, 31749, 31750, 31751, 31752) c6( ) c7(bxk, kC, ajU); #c1(NDUFAF5) c2(NP_077025) c3(NADH dehydrogenase [ubiquinone] 1 alpha subcomplex assembly factor 5 isoform 1) c4(214805) c5(214806, 214807, 214808, 214809, 214810, 214811, 214812) c6( ) c7(bxk, kC, ajU); #c1(NDUFAF5) c2(XP_011527643) c3(NADH dehydrogenase [ubiquinone] 1 alpha subcomplex assembly factor 5 isoform X1) c4(322560) c5( ) c6( ) c7(bxk, kC, ajU); #c1(NDUFAF5) c2(XP_011527645) c3(NADH dehydrogenase [ubiquinone] 1 alpha subcomplex assembly factor 5 isoform X3) c4( ) c5(322561, 322562, 322563, 322564, 322565) c6( ) c7(bxk, kC, ajU); #c1(NDUFAF6) c2(NP_689629) c3(NADH dehydrogenase (ubiquinone) complex I, assembly factor 6 precursor) c4(243346) c5(243347, 243348, 243349, 243350, 243351, 243352, 243353) c6( ) c7(bxk, aM, bxl, ajU); #c1(NDUFAF6) c2(XP_005250850) c3(NADH dehydrogenase (ubiquinone) complex I, assembly factor 6 isoform X4) c4( ) c5(270606, 270607, 270608, 270609, 270610) c6( ) c7(bxk, aM, bxl, ajU); #c1(NDUFAF6) c2(XP_011515144) c3(NADH dehydrogenase (ubiquinone) complex I, assembly factor 6 isoform X1) c4(307987, 307988) c5(307989, 307990, 307991, 307992, 307993, 307994) c6( ) c7(bxk, aM, bxl, ajU); #c1(NDUFAF6) c2(XP_011515146) c3(NADH dehydrogenase (ubiquinone) complex I, assembly factor 6 isoform X3) c4( ) c5(307995) c6( ) c7(bxk, aM, bxl, ajU); #c1(NDUFB10) c2(NP_004539) c3(NADH dehydrogenase [ubiquinone] 1 beta subcomplex subunit 10) c4(147419, 147420) c5(147421, 147422, 147423, 147424, 147425, 147426, 147427, 147428, 147429, 147430, 147431, 147432) c6( ) c7(cd); #c1(NDUFB11) c2(NP_001129470) c3(NADH dehydrogenase [ubiquinone] 1 beta subcomplex subunit 11, mitochondrial isoform 2) c4(51526, 51527) c5(51528, 51529, 51530, 51531, 51532, 51533, 51534, 51535, 51536, 51537, 51538, 51539) c6( ) c7(px); #c1(NDUFB11) c2(NP_061929) c3(NADH dehydrogenase [ubiquinone] 1 beta subcomplex subunit 11, mitochondrial isoform 1) c4(201194) c5(201195, 201196, 201197, 201198, 201199, 201200, 201201) c6( ) c7(px); #c1(NDUFB2) c2(NP_004537) c3(NADH dehydrogenase [ubiquinone] 1 beta subcomplex subunit 2, mitochondrial precursor) c4(147383, 147384, 147385) c5(147386, 147387, 147388, 147389, 147390, 147391, 147392, 147393, 147394, 147395, 147396, 147397, 147398, 147399, 147400, 147401, 147402, 147403, 147404, 147405, 147406, 147407, 147408, 147409, 147410, 147411) c6( ) c7(JQ, hD, lG, cy, ae, B, ad, vi); #c1(NDUFB3) c2(NP_001244031) c3(NADH dehydrogenase [ubiquinone] 1 beta subcomplex subunit 3) c4(91822, 91823) c5(91824, 91825, 91826, 91827, 91828, 91829, 91830, 91831, 91832, 91833, 91834, 91835, 91836, 91837, 91838, 91839, 91840, 91841, 91842, 91843) c6( ) c7(bxk, aW, my, aT); #c1(NDUFB4) c2(NP_001161803) c3(NADH dehydrogenase [ubiquinone] 1 beta subcomplex subunit 4 isoform 2) c4(66036) c5(66037, 66038, 66039, 66040, 66041, 66042) c6( ) c7(ao); #c1(NDUFB4) c2(NP_004538) c3(NADH dehydrogenase [ubiquinone] 1 beta subcomplex subunit 4 isoform 1) c4(147412) c5(147413, 147414, 147415, 147416, 147417, 147418) c6( ) c7(ao); #c1(NDUFB5) c2(NP_001186886) c3(NADH dehydrogenase [ubiquinone] 1 beta subcomplex subunit 5, mitochondrial isoform 2) c4(78757) c5(78758, 78759, 78760, 78761, 78762, 78763) c6( ) c7(ad); #c1(NDUFB5) c2(NP_001186887) c3(NADH dehydrogenase [ubiquinone] 1 beta subcomplex subunit 5, mitochondrial isoform 3 precursor) c4(78764) c5(78765, 78766, 78767, 78768, 78769) c6( ) c7(ad); #c1(NDUFB5) c2(NP_002483) c3(NADH dehydrogenase [ubiquinone] I beta subcomplex subunit 5, mitochondrial isoform 1 precursor) c4(129192) c5(129193, 129194, 129195, 129196, 129197, 129198, 129199) c6( ) c7(ad); #c1(NDUFB6) c2(NP_002484) c3(NADH dehydrogenase [ubiquinone] 1 beta subcomplex subunit 6 isoform 1) c4(129200) c5(129201, 129202, 129203, 129204, 129205, 129206) c6( ) c7(bL, ae, aM, ac, ad); #c1(NDUFB6) c2(NP_877416) c3(NADH dehydrogenase [ubiquinone] 1 beta subcomplex subunit 6 isoform 2) c4(256758) c5(256759, 256760, 256761, 256762, 256763, 256764) c6( ) c7(bL, ae, aM, ac, ad); #c1(NDUFB8) c2(NP_001271296) c3(NADH dehydrogenase [ubiquinone] 1 beta subcomplex subunit 8, mitochondrial isoform 2) c4(103867) c5(103868, 103869, 103870, 103871, 103872, 103873, 103874, 103875, 103876, 103877) c6( ) c7(Pk, lQ, ad); #c1(NDUFB8) c2(NP_001271297) c3(NADH dehydrogenase [ubiquinone] 1 beta subcomplex subunit 8, mitochondrial isoform 3) c4( ) c5(103878, 103879, 103880, 103881, 103882) c6( ) c7(Pk, lQ, ad); #c1(NDUFB8) c2(NP_004995) c3(NADH dehydrogenase [ubiquinone] 1 beta subcomplex subunit 8, mitochondrial isoform 1 precursor) c4(151835, 151836) c5(151837, 151838, 151839, 151840, 151841, 151842, 151843) c6( ) c7(Pk, lQ, ad); #c1(NDUFB9) c2(NP_004996) c3(NADH dehydrogenase [ubiquinone] 1 beta subcomplex subunit 9 isoform 1) c4(151844) c5(151845, 151846, 151847, 151848, 151849, 151850) c6( ) c7(bxk, aM, ad); #c1(NDUFC2) c2(NP_001190983) c3(NADH dehydrogenase [ubiquinone] 1 subunit CZ isoform 2) c4(80380) c5(80381, 80382, 80383, 80384, 80385, 80386, 80387, 80388, 80389, 80390, 80391) c6( ) c7(bz, ad); #c1(NDUFC2) c2(NP_001190984) c3(NADH dehydrogenase [ubiquinone] 1 subunit CZ isoform 3) c4(80392) c5(80393, 80394, 80395, 80396, 80397, 80398, 80399) c6( ) c7(bz, ad); #c1(NDUFC2) c2(NP_004540) c3(NADH dehydrogenase [ubiquinone] 1 subunit CZ isoform 1) c4(147433, 147434) c5(147435, 147436, 147437, 147438, 147439, 147440) c6( ) c7(bz, ad); #c1(NDUFS1) c2(NP_001186910) c3(NADH-ubiquinone oxidoreductase 75 kDa subunit, mitochondrial isoform 2 precursor) c4(78770, 78771) c5(78772, 78773, 78774, 78775, 78776, 78777) c6( ) c7(cjf, aM, aXb, ajU, aA, bxk, abn, vc, K, rz, ad, bN); #c1(NDUFS1) c2(NP_001186911) c3(NADH-ubiquinone oxidoreductase 75 kDa subunit, mitochondrial isoform 3) c4(78778) c5(78779, 78780, 78781, 78782, 78783, 78784) c6( ) c7(cjf, aM, aXb, ajU, aA, bxk, abn, vc, K, rz, ad, bN); #c1(NDUFS1) c2(NP_001186913) c3(NADH-ubiquinone oxidoreductase 75 kDa subunit, mitochondrial isoform 5) c4(78785, 78786, 78787, 78788) c5(78789, 78790, 78791, 78792, 78793, 78794, 78795, 78796, 78797, 78798, 78799, 78800, 78801, 78802, 78803, 78804, 78805, 78806, 78807, 78808, 78809, 78810, 78811, 78812, 78813, 78814, 78815, 78816, 78817) c6( ) c7(cjf, aM, aXb, ajU, aA, bxk, abn, vc, K, rz, ad, bN); #c1(NDUFS1) c2(NP_004997) c3(NADH-ubiquinone oxidoreductase 75 kDa subunit, mitochondrial isoform 1 precursor) c4( ) c5(151851, 151852, 151853, 151854, 151855) c6( ) c7(cjf, aM, aXb, ajU, aA, bxk, abn, vc, K, rz, ad, bN); #c1(NDUFS2) c2(NP_001159631) c3(NADH dehydrogenase [ubiquinone] iron-sulfur protein 2, mitochondrial isoform 2 precursor) c4(64483, 64484) c5(64485, 64486, 64487, 64488, 64489, 64490, 64491) c6( ) c7(T, cc, dc, aM, jZ, ajU, bxk, bq, px, V, ad, ip); #c1(NDUFS2) c2(NP_004541) c3(NADH dehydrogenase [ubiquinone] iron-sulfur protein 2, mitochondrial isoform 1 precursor) c4(147441) c5(147442, 147443, 147444, 147445, 147446, 147447, 147448) c6( ) c7(T, cc, dc, aM, jZ, ajU, bxk, bq, px, V, ad, ip); #c1(NDUFS2) c2(XP_005245266) c3(NADH dehydrogenase [ubiquinone] iron-sulfur protein 2, mitochondrial isoform X2) c4(266367) c5(266368, 266369, 266370, 266371, 266372, 266373, 266374, 266375, 266376, 266377) c6( ) c7(T, cc, dc, aM, jZ, ajU, bxk, bq, px, V, ad, ip); #c1(NDUFS3) c2(NP_004542) c3(NADH dehydrogenase [ubiquinone] iron-sulfur protein 3, mitochondrial precursor) c4(147449) c5(147450, 147451, 147452, 147453, 147454, 147455) c6( ) c7(es, cc, eM, sO, aM, aT, awk, bxl, bq, f, ajU); #c1(NDUFS4) c2(NP_002486) c3(NADH dehydrogenase [ubiquinone] iron-sulfur protein 4, mitochondrial precursor) c4(129207) c5(129208, 129209, 129210, 129211, 129212, 129213) c6( ) c7(y, W, eR, hb, aM, ajU, hL, r, bxk, aDa, cl, bxl, eZ, an, px, q, aR, ad, cjg); #c1(NDUFS6) c2(NP_004544) c3 (NADH dehydrogenase [ubiquinone] iron-sulfur protein 6, mitochondrial precursor) c4(147456) c5(147457, 147458, 147459, 147460, 147461, 147462) c6( ) c7(bxk, if, aM, ad, qN); #c1(NDUFS7) c2(NP_077718) c3(NADH dehydrogenase [ubiquinone] iron-sulfur protein 7, mitochondrial) c4(215248, 215249, 215250) c5(215251, 215252, 215253, 215254, 215255, 215256, 215257, 215258, 215259, 215260, 215261, 215262, 215263, 215264, 215265, 215266, 215267, 215268, 215269, 215270, 215271, 215272, 215273) c6( ) c7(ac, Y, aM, ajU, bxk, aj, bxl, J); #c1(NDUFS7) c2(XP_005259613) c3(NADH dehydrogenase [ubiquinone] iron-sulfur protein 7, mitochondrial isoform X1) c4(276200) c5(276201, 276202, 276203, 276204, 276205, 276206) c6( ) c7(ac, Y, aM, ajU, bxk, aj, bxl, J); #c1(NDUFS8) c2(NP_002487) c3(NADH dehydrogenase [ubiquinone] iron-sulfur protein 8, mitochondrial precursor) c4(129214, 129215, 129216, 129217, 129218) c5(129219, 129220, 129221, 129222, 129223, 129224, 129225, 129226, 129227, 129228, 129229, 129230, 129231, 129232, 129233, 129234, 129235, 129236, 129237, 129238, 129239, 129240, 129241, 129242) c6( ) c7(bxk, aM, bxl, ajU); #c1(NDUFS8) c2(XP_005274072) c3(NADH dehydrogenase [ubiquinone] iron-sulfur protein 8, mitochondrial isoform X2) c4( ) c5(284928, 284929, 284930, 284931, 284932, 284933) c6( ) c7(bxk, aM, bxl, ajU); #c1(NDUFV1) c2(NP_001159574) c3(NADH dehydrogenase [ubiquinone] flavoprotein 1, mitochondrial isoform 2 precursor) c4(64283) c5(64284, 64285, 64286, 64287, 64288, 64289, 64290, 64291, 64292, 64293, 64294, 64295, 64296, 64297, 64298, 64299, 64300, 64301) c6( ) c7(EG, aM, ajU, aA, bxk, asF, cb, kC, aR); #c1(NDUFV1) c2(NP_009034) c3(NADH dehydrogenase [ubiquinone] flavoprotein 1, mitochondrial isoform 1 precursor) c4(170326, 170327, 170328, 170329) c5(170330, 170331, 170332, 170333, 170334, 170335, 170336, 170337, 170338, 170339, 170340, 170341) c6( ) c7(EG, aM, ajU, aA, bxk, asF, cb, kC, aR); #c1(NDUFV2) c2(NP_066552) c3(NADH dehydrogenase [ubiquinone] flavoprotein 2, mitochondrial precursor) c4(207381) c5(207382, 207383, 207384, 207385, 207386, 207387) c6( ) c7(cc, hb, aM, ajU, bxk, vc, bK, an, oi, px, ad, GB); #c1(NDUFV3) c2(NP_001001503) c3(NADH dehydrogenase [ubiquinone] flavoprotein 3, mitochondrial isoform b precursor) c4(18553) c5(18554, 18555, 18556, 18557, 18558, 18559, 18560, 18561, 18562, 18563, 18564, 18565) c6( ) c7(f, ad); #c1(NDUFV3) c2(NP_066553) c3(NADH dehydrogenase [ubiquinone] flavoprotein 3, mitochondrial isoform a precursor) c4(207388) c5(207389, 207390, 207391, 207392, 207393, 207394) c6( ) c7(f, ad); #c1(NDUFV3) c2(XP_011527888) c3(NADH dehydrogenase [ubiquinone] flavoprotein 3, mitochondrial isoform X1) c4(323002) c5( ) c6( ) c7(f, ad); #c1(NEB) c2(NP_001157979) c3(nebulin isoform 1) c4(62981) c5(62982, 62983, 62984, 62985, 62986, 62987, 62988) c6( ) c7(cc, no, Ag, Ws, Am, xs, lb, oj, Ad, Ak, uY, bl); #c1(NEB) c2(NP_001157980) c3(nebulin isoform 2) c4(62989) c5(62990, 62991, 62992, 62993, 62994, 62995, 62996) c6( ) c7(cc, no, Ag, Ws, Am, xs, lb, oj, Ad, Ak, uY, bl); #c1(NEB) c2(NP_001258137) c3(nebulin isoform 4) c4( ) c5(95979, 95980, 95981, 95982, 95983, 95984, 95985, 95986, 95987, 95988, 95989, 95990) c6( ) c7(cc, no, Ag, Ws, Am, xs, lb, oj, Ad, Ak, uY, bl); #c1(NEB) c2(NP_004534) c3(nebulin isoform 3) c4(147353, 147354) c5(147355, 147356, 147357, 147358, 147359, 147360, 147361, 147362, 147363, 147364, 147365, 147366, 147367, 147368) c6( ) c7(cc, no, Ag, Ws, Am, xs, lb, oj, Ad, Ak, uY, bl); #c1(NEB) c2(XP_005246653) c3(nebulin isoform X8) c4(267303) c5(267304, 267305, 267306, 267307, 267308, 267309) c6( ) c7(cc, no, Ag, Ws, Am, xs, lb, oj, Ad, Ak, uY, bl); #c1(NEB) c2(XP_005246663) c3(nebulin isoform X17) c4(267310) c5( ) c6( ) c7(cc, no, Ag, Ws, Am, xs, lb, oj, Ad, Ak, uY, bl); #c1(NEB) c2(XP_005246667) c3(nebulin isoform X21) c4(267311) c5(267312, 267313, 267314, 267315, 267316, 267317) c6( ) c7(cc, no, Ag, Ws, Am, xs, lb, oj, Ad, Ak, uY, bl); #c1(NEB) c2(XP_005246670) c3(nebulin isoform X24) c4(267318) c5( ) c6( ) c7(cc, no, Ag, Ws, Am, xs, lb, oj, Ad, Ak, uY, bl); #c1(NEB) c2(XP_005246672) c3(nebulin isoform X26) c4(267319) c5(267320, 267321, 267322, 267323, 267324, 267325) c6( ) c7(cc, no, Ag, Ws, Am, xs, lb, aj, Ad, Ak, uY, bl); #c1(NEBL) c2(NP_006384) c3(nebulette isoform 1) c4(164553) c5(164554, 164555, 164556, 164557, 164558, 164559) c6( ) c7(vc, acC, mx, Bt); #c1(NEBL) c2(NP_998734) c3(nebulette isoform 2) c4(265804, 265805) c5(265806, 265807, 265808, 265809, 265810, 265811, 265812, 265813, 265814, 265815, 265816, 265817) c6( ) c7(vc, acC, mx, Bt); #c1(NEBL) c2(XP_005252400) c3(nebulette isoform X3) c4(271714) c5( ) c6( ) c7(vc, acC, mx, Bt); #c1(NEBL) c2(XP_005252401) c3(nebulette isoform X4) c4( ) c5(271715, 271716, 271717, 271718, 271719, 271720) c6( ) c7(vc, acC, mx, Bt); #c1(NEBL) c2(XP_011517593) c3(nebulette isoform X1) c4(311348) c5(311349, 311350, 311351, 311352, 311353, 311354) c6( ) c7(vc, acC, mx, Bt); #c1(NECAB1) c2(NP_071746) c3(N-terminal EF-hand calcium-binding protein 1) c4(211914, 211915) c5(211916, 211917, 211918, 211919, 211920, 211921, 211922, 211923, 211924, 211925, 211926, 211927, 211928, 211929, 211930, 211931) c6( ) c7(U); #c1(NECAB3) c2(NP_112508) c3(N-terminal EF-hand calcium-binding protein 3 isoform 1) c4(221316) c5(221317, 221318, 221319, 221320, 221321, 221322) c6( ) c7(aW, aT); #c1(NECAB3) c2(NP_112509) c3(N-terminal EF-hand calcium-binding protein 3 isoform 2) c4(221323) c5(221324, 221325, 221326, 221327, 221328, 221329) c6( ) c7(aW, aT); #c1(NECAB3) c2(XP_005260567) c3(N-terminal EF-hand calcium-binding protein 3 isoform X1) c4(276633) c5(276634, 276635, 276636, 276637, 276638, 276639) c6( ) c7(aW, aT); #c1(NECAB3) c2(XP_011527294) c3(N-terminal EF-hand calcium-binding protein 3 isoform X4) c4( ) c5(321918) c6( ) c7(aW, aT); #c1(NECAP1) c2(NP_056324) c3(adaptin ear-binding coat-associated protein 1) c4(187068, 187069) c5(187070, 187071, 187072, 187073, 187074, 187075, 187076, 187077, 187078, 187079, 187080, 187081, 187082, 187083, 187084, 187085) c6( ) c7(lm); #c1(NEDD1) c2(NP_001128648) c3(protein NEDD1 isoform b) c4(50441, 50442) c5(50443, 50444, 50445, 50446, 50447, 50448, 50449, 50450, 50451, 50452, 50453) c6( ) c7(aF, aA, pN); #c1(NEDD1) c2(XP_005268701) c3 (protein NEDD1 isoform X1) c4(282054, 282055, 282056) c5(282057, 282058, 282059, 282060, 282061, 282062, 282063, 282064, 282065, 282066, 282067, 282068, 282069, 282070, 282071, 282072, 282073, 282074, 282075, 282076, 282077, 282078, 282079, 282080, 282081, 282082) c6( ) c7(aF, aA, pN); #c1(NEDD1) c2(XP_011536205) c3(protein NEDD1 isoform X2) c4(333505, 333506) c5(333507, 333508, 333509, 333510, 333511, 333512, 333513, 333514, 333515, 333516, 333517, 333518, 333519) c6( ) c7(aF, aA, pN); #c1(NEDD4) c2(NP_001271267) c3(E3 ubiquitin-protein ligase NEDD4 isoform 3) c4(103785) c5(103786, 103787, 103788, 103789, 103790, 103791) c6( ) c7(T, aA, j, cd, eA, q, ad, hX, aT, h, bk, K, bl, bU, ae, aW, r, bZC, bz, eM, U, cj); #c1(NEDD4) c2(NP_001271268) c3(E3 ubiquitin-protein ligase NEDD4 isoform 4) c4(103792) c5(103793, 103794, 103795, 103796, 103797, 103798) c6( ) c7(T, aA, j, cd, eA, q, ad, hX, aT, h, bK, K, bl, bU, ae, aW, r, bZC, bz, eM, U, cj); #c1(NEDD4) c2(NP_006145) c3(E3 ubiquitin-protein ligase NEDD4 isoform 1) c4(162398) c5(162399, 162400, 162401, 162402, 162403, 162404) c6( ) c7(T, aA, j, cd, eA, q, ad, hX, aT, h, bK, K, bl, bU, ae, aW, r, bZC, bz, eM, U, cj); #c1(NEDD4) c2(NP_940682) c3(E3 ubiquitin-protein ligase NEDD4 isoform 2) c4(259836) c5(259837, 259838, 259839, 259840, 259841, 259842) c6( ) c7(T, aA, j, cd, eA, q, ad, hX, aT, h, bK, K, bl, bU, ae, aW, r, bZC, bz, eM, U, cj); #c1(NEDD4L) c2(NP_001138439) c3(E3 ubiquitin-protein ligase NEDD4-like isoform 1) c4(56768, 56769) c5(56770, 56771, 56772, 56773, 56774, 56775) c6( ) c7(cjh, qa, T, dP, bM, ac, eL, e, ha, nl, nh, cz, MF, cji, v, hg, yA, ae, ad); #c1(NEDD4L) c2(NP_001138440) c3(E3 ubiquitin-protein ligase NEDD4-like isoform 4) c4(56776) c5(56777, 56778, 56779, 56780, 56781, 56782, 56783, 56784) c6( ) c7(cjh, qa, T, dP, bM, ac, eU, e, ha, nl, nh, cz, MF, cji, v, hg, yA, ae, ad); #c1(NEDD4L) c2(NP_001138441) c3(E3 ubiquitin-protein ligase NEDD4-like isoform 5) c4( ) c5(56785) c6( ) c7(cjh, qa, T, dP, bM, ac, eU, e, ha, nl, nh, cz, MF, cji, v, hg, yA, ae, ad); #c1(NEDD4L) c2(NP_00138442) c3(E3 ubiquitin-protein ligase NEDD4-like isoform 6) c4( ) c5(56786, 56787, 56788, 56789, 56790, 56791, 56792, 56793, 56794, 56795, 56796, 56797, 56798) c6( ) c7(cjh, qa, T, dP, bM, ac, eU, e, ha, nl, nh, cz, MF, cji, v, hg, yA, ae, ad); #c1(NEDD4L) c2(NP_001230889) c3(E3 ubiquitin-protein ligase NEDD4-like isoform 7) c4(86929, 86930) c5(86931, 86932, 86933, 86934, 86935, 86936, 86937, 86938, 86939, 86940, 86941, 86942) c6( ) c7(cjh, qa, T, dP, bM, ac, eU, e, ha, nl, nh, cz, MF, cji, v, hg, yA, ae, ad); #c1(NEDD4L) c2(NP_056092) c3(E3 ubiquitin-protein ligase NEDD4-like isoform 3) c4(185670, 185671) c5(185672, 185673, 185674, 185675, 185676, 185677, 185678) c6( ) c7(cjh, qa, T, dP, bM, ac, eL, e, ha, nl, nh, cz, MF, cji, v, hg, yA, ae, ad); #c1(NEDD4L) c2(XP_005266717) c3(E3 ubiquitin-protein ligase NEDD4-like isoform X2) c4( ) c5(280280, 280281, 280282, 280283, 280284, 280285, 280286, 280287) c6( ) c7(cjh, qa, T, dP, bM, ac, eL, e, ha, nl, nh, cz, MF, cji, v, hg, yA, ae, ad); #c1(NEDD4L) c2(XP_005266720) c3(E3 ubiquitin-protein ligase NEDD4-like isoform X4) c4(280288) c5( ) c6( ) c7(cjh, qa, T, dP, bM, ac, eU, e, ha, nl, nh, cz, MF, cji, v, hg, yA, ae, ad); #c1(NEDD4L) c2(XP_011524189) c3(E3 ubiquitin-protein ligase NEDD4-like isoform X8) c4( ) c5(318734) c6( ) c7(cjh, qa, T, dP, bM, ac, eL, e, ha, nl, nh, cz, MF, cji, v, hg, yA, ae, ad); #c1(NEDD8) c2(NP_006147) c3(NEDD8 precursor) c4(162405) c5(162406, 162407, 162408, 162409, 162410, 162411) c6( ) c7(T, GG, aW, ff, cr, aT, h, fd, K, m, anW, en, t, bt, GR); #c1(NEDD9) c2(NP_001135865) c3(enhancer of filamentation 1 isoform 3) c4(53427, 53428) c5(53429, 53430, 53431, 53432, 53433, 53434, 53435, 53436, 53437, 53438, 53439, 53440) c6( ) c7(T, iW, aw, jh, aA, bB, n, fr, ci, qN, alq, q, ai, eA, x, av, Bl, aE, anV, aT, h, hn, an, fb, af, d, s, aW, Y, ff, r, kp, aF, en, iV, t, lk, bz, cm, iK, ck, kP, eM, bP, bE, iT, adR, np, m, k, aps, bt, B, y); #c1(NEDD9) c2(NP_006394) c3(enhancer of filamentation 1 isoform 1) c4(164663) c5(164664, 164665, 164666, 164667, 164668, 164669) c6( ) c7(T, iW, aw, jh, aA, bB, n, fr, ci, qN, alq, q, ai, eA, x, av, Bl, aE, anV, aT, h, hn, an, fb, af, d, s, aW, Y, ff, r, kp, aF, en, iV, t, lk, bz, cm, iK, ck, kP, eM, bP, bE, iT, adR, np, m, k, aps, bt, B, y); #c1(NEDD9) c2(NP_892011) c3(enhancer of filamentation 1 isoform 2) c4(257590) c5(257591, 257592, 257593, 257594, 257595, 257596) c6( ) c7(T, iW, aw, jh, aA, bB, n, fr, ci, qN, alq, q, ai, eA, x, av, Bl, aE, anV, aT, h, hn, an, fb, af, d, s, aW, Y, ff, r, kp, aF, en, iV, t, lk, bz, cm, iK, ck, kP, eM, bP, bE, iT, adR, np, m, k, aps, bt, B, y); #c1(NEFH) c2(NP_066554) c3(neurofilament heavy polypeptide) c4(207395, 207396) c5( ) c6( ) c7(qa, aQy, T, bb, Ew, c, OI, My, U, cz, acU, K, m, bq, PJ, t, bX, it, af, aR, f); #c1(NEFH) c2(XP_011528502) c3(neurofilament heavy polypeptide isoform X1) c4( ) c5(323597, 323598, 323599, 323600, 323601, 323602, 323603, 323604, 323605, 323606, 323607, 323608) c6( ) c7(qa, aQy, T, bb, Ew, c, OI, My, U, cz, acU, K, m, bq, PJ, t, bX, it, af, aR, f); #c1(NEFM) c2(NP_001099011) c3(neurofilament medium polypeptide isoform 2) c4(41648) c5(41649, 41650, 41651, 41652, 41653, 41654) c6( ) c7(dP, bb, c, OI, an, KP, alQ, La, bq, aQy); #c1(NEFM) c2(NP_005373) c3(neurofilament medium polypeptide isoform 1) c4(155544, 155545) c5(155546, 155547, 155548, 155549, 155550, 155551, 155552, 155553, 155554, 155555, 155556, 155557) c6( ) c7(dP, bb, c, OI, an, KP, alQ, La, bq, aQy); #c1(NEIL1) c2(NP_001243481) c3(endonuclease 8-like 1 isoform 1) c4( ) c5(90929) c6( ) c7(T, os, sw, aOE, aA, oZ, v, fr, V, jz, It, aT, h, jy, K, dQ, ff, fc, hl, hN, aF, en, iK, bF, es, eM, mf, ei); #c1(NEIL1) c2(NP_078884) c3(endonuclease 8-like 1 isoform 2) c4( ) c5(215894, 215895, 215896, 215897, 215898, 215899, 215900, 215901, 215902, 215903, 215904, 215905, 215906, 215907) c6( ) c7(T, os, sw, aOE, aA, oZ, v, fr, V, jz, lt, aT, h, jy, K, dQ, ff, fc, hl, hN, aF, en, iK, bF, es, eM, mf, ei); #c1(NEIL1) c2(XP_006720740) c3 (endonuclease 8-like 1 isoform X2) c4(293660) c5( ) c6( ) c7(T, os, sw, aOE, aA, oZ, v, fr, V, jz, lt, aT, h, jy, K, dQ, ff, fc, hl, hN, aF, en, iK, bF, es, eM, mf, ei); #c1(NEIL1) c2(XP_006720742) c3(endonuclease 8-like 1 isoform X4) c4(293661) c5(293662) c6( ) c7(T, os, sw, aOE, aA, oZ, v, fr, V, jz, lt, aT, h, jy, K, dQ, ff, fc, hl, hN, aF, en, iK, bF, es, eM, mf, ei); #c1(NEIL2) c2(NP_001129219) c3(endonuclease 8-like 2 isoform b) c4( ) c5(51238, 51239, 51240, 51241, 51242) c6( ) c7(bF, T, BN, sw, aW, ff, aoE, al, r, n, hy, GA, m, q, en, t, V, bz, ne); #c1(NEIL2) c2(NP_001129220) c3(endonuclease 8-like 2 isoform c) c4(51243) c5(51244, 51245, 51246, 51247, 51248, 51249) c6( ) c7(bF, T, BN, sw, aW, ff, aOE, aT, r, n, hy, GA, m, q, en, t, V, bz, ne); #c1(NEIL2) c2(NP_659480) c3(endonuclease 8-like 2 isoform a) c4(240113, 240114, 240115) c5(240116, 240117, 240118, 240119, 240120, 240121, 240122, 240123, 240124, 240125, 240126, 240127, 240128, 240129, 240130, 240131, 240132, 240133, 240134, 240135, 240136) c6( ) c7(bF, T, BN, sw, aW, ff, aoE, aT, r, n, hy, GA, m, q, en, t, V, bz, ne); #c1(NEIL3) c2(NP_060718) c3(endonuclease 8-like 3) c4(197454) c5(197455, 197456, 197457, 197458, 197459, 197460) c6( ) c7(bF, ey, bX); #c1(NEK1) c2(NP_001186326) c3(serine/threonine-protein kinase Nek1 isoform 1) c4(77472) c5(77473, 77474, 77475, 77476, 77477, 77478) c6( ) c7(T, wB, aT, aA, aF, bWB, ci, zG, ade, rv, cjj); #c1(NEK1) c2(NP_001186327) c3(serine/threonine-protein kinase Nek1 isoform 3) c4(77479) c5(77480, 77481, 77482, 77483, 77484, 77485) c6( ) c7(T, wB, aT, aA, aF, bWB, ci, zG, ade, rv, cjj); #c1(NEK1) c2(NP_001186328) c3(serine/threonine-protein kinase Nek1 isoform 4) c4(77486) c5(77487, 77488, 77489, 77490, 77491, 77492) c6( ) c7(T, wB, aT, aA, aF, bWB, ci, zG, ade, rv, cjj); #c1(NEK1) c2(NP_001186329) c3(serine/threonine-protein kinase Nek1 isoform 5) c4(77493) c5(77494, 77495, 77496, 77497, 77498, 77499) c6( ) c7(T, wB, aT, aA, aF, bWB, ci, zG, ade, rv, cjj); #c1(NEK1) c2(NP_036356) c3(serine/threonine-protein kinase Nek1 isoform 2) c4(173197) c5(173198, 173199, 173200, 173201, 173202, 173203) c6( ) c7(T, wB, aT, aA, aF, bWB, ci, zG, ade, rv, cjj); #c1(NEK10) c2(NP_001026911) c3(serine/threonine-protein kinase Nek10 isoform 1) c4(29976) c5(29977, 29978, 29979, 29980, 29981, 29982, 29983, 29984, 29985, 29986, 29987, 29988) c6( ) c7(aT); #c1(NEK10) c2(NP_955379) c3(serine/threonine-protein kinase Nek10 isoform 3) c4(261616, 261617) c5(261618, 261619, 261620, 261621, 261622, 261623, 261624, 261625, 261626, 261627, 261628, 261629) c6( ) c7(aT); #c1(NEK10) c2(XP_006713064) c3(serine/threonine-protein kinase Nek10 isoform X9) c4(287008) c5( ) c6( ) c7(aT); #c1(NEK10) c2(XP_011531712) c3(serine/threonine-protein kinase Nek10 isoform X6) c4(327794) c5(327795, 327796, 327797, 327798, 327799, 327800) c6( ) c7(aT); #c1(NEK10) c2(XP_011531714) c3(serine/threonine-protein kinase Nek10 isoform X8) c4(327801, 327802) c5( ) c6( ) c7(aT); #c1(NEK10) c2(XP_011531717) c3(serine/threonine-protein kinase Nek10 isoform X13) c4( ) c5(327803, 327804, 327805, 327806, 327807, 327808, 327809, 327810, 327811, 327812, 327813, 327814) c6( ) c7(aT); #c1(NEK11) c2(NP_001139475) c3(serine/threonine-protein kinase Nek11 isoform 3) c4(59393) c5(59394, 59395, 59396, 59397, 59398, 59399) c6( ) c7(t, T, m); #c1(NEK11) c2(NP_079076) c3(serine/threonine-protein kinase Nek11 isoform 1) c4(216907) c5(216908, 216909, 216910, 216911, 216912, 216913, 216914, 216915, 216916, 216917, 216918, 216919, 216920, 216921) c6( ) c7(t, T, m); #c1(NEK11) c2(NP_665917) c3(serine/threonine-protein kinase Nek11 isoform 2) c4(241991) c5(241992, 241993, 241994, 241995, 241996, 241997) c6( ) c7(t, T, m); #c1(NEK11) c2(XP_011511472) c3(serine/threonine-protein kinase Nek11 isoform X3) c4(302150) c5(302151) c6( ) c7(t, T, m); #c1(NEK11) c2(XP_011511481) c3(serine/threonine-protein kinase Nek11 isoform X8) c4(302152) c5(302153, 302154, 302155, 302156, 302157, 302158, 302159, 302160, 302161, 302162) c6( ) c7(t, T, m); #c1(NEK11) c2(XP_011511484) c3(serine/threonine-protein kinase Nek11 isoform X11) c4( ) c5(302163, 302164, 302165, 302166, 302167) c6( ) c7(t, T, m); #c1(NEK2) c2(NP_001191112) c3(serine/threonine-protein kinase Nek2 isoform 2) c4(81041) c5(81042, 81043, 81044, 81045, 81046, 81047) c6( ) c7(T, wu, fd, n, ci, AO, q, jz, cjk, aE, aT, h, wm, aEL, B, qt, aW, r, nY, mo, t, kP, eM, m, iu); #c1(NEK2) c2(NP_002488) c3(serine/threonine-protein kinase Nek2 isoform 1) c4(129243, 129244) c5(129245, 129246, 129247, 129248, 129249, 129250, 129251, 129252, 129253, 129254, 129255, 129256) c6( ) c7(T, wu, fd, n, ci, AO, q, jz, cjk, aE, aT, h, wm, aEL, B, qt, aW, r, nY, mo, t, kP, eM, m, iu); #c1(NEK3) c2(NP_002489) c3(serine/threonine-protein kinase Nek3 isoform a) c4(129257, 129258) c5(129259, 129260, 129261, 129262, 129263, 129264, 129265, 129266, 129267, 129268) c6( ) c7(aW, aT); #c1(NEK4) c2(NP_001180462) c3(serine/threonine-protein kinase Nek4 isoform 2) c4(74131, 74132, 74133) c5(74134, 74135, 74136, 74137, 74138, 74139) c6( ) c7(eM, ey, bz, bK, bX); #c1(NEK4) c2(NP_003148) c3(serine/threonine-protein kinase Nek4 isoform 1) c4(135717, 135718) c5(135719, 135720, 135721, 135722, 135723, 135724) c6( ) c7(eM, ey, bz, bK, bX); #c1(NEK4) c2(XP_011532342) c3(serine/threonine-protein kinase Nek4 isoform X3) c4( ) c5(328494, 328495, 328496, 328497, 328498) c6( ) c7(eM, ey, bz, bK, bX); #c1(NEK6) c2(NP_001138473) c3(serine/threonine-protein kinase Nek6 isoform 1) c4(56834, 56835) c5(56836, 56837, 56838, 56839, 56840, 56841, 56842, 56843, 56844, 56845, 56846, 56847, 56848, 56849, 56850, 56851) c6( ) c7(T, cF, aj, dL, MC, eL, B, dR); #c1(NEK6) c2(NP_001159639) c3(serine/threonine-protein kinase Nek6 isoform 3) c4(64516, 64517) c5(64518, 64519, 64520, 64521, 64522, 64523, 64524, 64525) c6( ) c7(T, cF, aj, dL, MC, eL, B, dR); #c1(NEK6) c2(NP_001159641) c3(serine/threonine-protein kinase Nek6 isoform 4) c4(64526, 64527, 64528, 64529, 64530, 64531) c5(64532, 64533, 64534, 64535, 64536, 64537, 64538, 64539, 64540, 64541, 64542, 64543) c6( ) c7(T, cF, aj, dL, MC, eL, B, dR); #c1(NEK6) c2(XP_006716999) c3(serine/threonine-protein kinase Nek6 isoform X1) c4( ) c5(290610, 290611, 290612, 290613, 290614, 290615, 290616, 290617, 290618, 290619, 290620, 290621, 290622, 290623, 290624, 290625, 290626, 290627, 290628, 290629, 290630) c6( ) c7(T, cF, aj, dL, MC, eL, B, dR); #c1(NEK6) c2(XP_011516457) c3(serine/threonine-protein kinase Nek6 isoform X2) c4(309769, 309770) c5(309771, 309772, 309773, 309774, 309775, 309776, 309777, 309778, 309779, 309780, 309781, 309782, 309783, 309784, 309785, 309786, 309787, 309788, 309789, 309790, 309791, 309792, 309793, 309794, 309795, 309796, 309797, 309798, 309799, 309800, 309801) c6( ) c7(T, cF, aj, dL, MC, eL, B, dR); #c1(NEK7) c2(NP_598001) c3(serine/threonine-protein kinase Nek7) c4(235257) c5(235258, 235259, 235260, 235261, 235262, 235263, 235264, 235265, 235266, 235267) c6( ) c7(aT); #c1(NEK7) c2(XP_011507510) c3(serine/threonine-protein kinase Nek7 isoform X1) c4(297440, 297441, 297442) c5( ) c6( ) c7(al); #c1(NEK7) c2(XP_011507511) c3(serine/threonine-protein kinase Nek7 isoform X2) c4( ) c5(297443, 297444, 297445, 297446, 297447, 297448, 297449, 297450, 297451, 297452, 297453, 297454, 297455, 297456, 297457, 297458, 297459, 297460) c6( ) c7(aT); #c1(NEK8) c2(NP_835464) c3(serine/threonine-protein kinase Nek8) c4(253525) c5(253526, 253527, 253528, 253529, 253530, 253531) c6( ) c7(d, aE, bE, aW, wB, aT, vm, dy, cjm, K, Nj, aBZ, cjl, vN); #c1(NEK8) c2(XP_011522947) c3(serine/threonine-protein kinase Nek8 isoform X6) c4(317442) c5(317443, 317444, 317445, 317446, 317447, 317448) c6( ) c7(d, aE, bE, aW, wB, aT, vm, dy, cjm, K, Nj, aBZ, cjl, vN); #c1(NEK9) c2(NP_149107) c3(serine/threonine-protein kinase Nek9) c4(227185) c5(227186, 227187, 227188, 227189, 227190, 227191) c6( ) c7(wB, aT, vm, aBZ, Nj, K, VN); #c(NEK9) cZ(XP_005268265) c3(serine/threonine-protein kinase Nek9 isoform X1) c4(281653) c5(281654, 281655, 281656, 281657, 281658, 281659) c6( ) c7(wB, aT, vm, aBZ, Nj, K, vN); #c1(NELFA) c2(NP_005654) c3(negative elongation factor A) c4(158294, 158295) c5(158296, 158297, 158298, 158299, 158300, 158301, 158302, 158303, 158304, 158305, 158306, 158307, 158308, 158309, 158310, 158311, 158312, 158313) c6( ) c7(axC); #c1(NELFA) c2(XP_006713979) c3(negative elongation factor A isoform X2) c4(287488) c5( ) c6( ) c7(axC); #c1(NELFB) c2(NP_056271) c3(negative elongation factor B) c4(186842) c5(186843, 186844, 186845, 186846, 186847, 186848) c6( ) c7(T, aW, aA, aT, aF, ci, g); #c1(NELFCD) c2(NP_945327) c3(negative elongation factor C/D) c4(260818) c5(260819, 260820, 260821, 260822, 260823, 260824, 260825, 260826, 260827, 260828) c6( ) c7(A, lg, Pg, aGd, BG, y); #c1(NELFE) c2(NP_002895) c3(negative elongation factor E) c4(133295, 133296, 133297, 133298) c5(133299, 133300, 133301, 133302, 133303, 133304, 133305, 133306, 133307, 133308, 133309, 133310, 133311, 133312, 133313, 133314, 133315, 133316, 133317, 133318, 133319, 133320, 133321, 133322, 133323, 133324, 133325, 133326, 133327, 133328, 133329, 133330, 133331, 133332, 133333, 133334, 133335, 133336, 133337, 133338, 133339, 133340, 133341, 133342, 133343, 133344, 133345, 133346, 133347, 133348, 133349, 133350, 133351, 133352, 133353, 133354, 133355, 133356, 133357, 133358, 133359, 133360, 133361, 133362, 133363, 133364, 133365, 133366, 133367, 133368, 133369, 133370, 133371, 133372, 133373, 133374, 133375, 133376, 133377, 133378, 133379, 133380, 133381, 133382, 133383) c6( ) c7(T, os, aC, bB, gA, ci, x, aU, K, hu, H, eL, bc, aW, La, as, eE, cm, akw, aAW, eM, nB, iT, vA, U, dW, nw, k, m, avU, NK, gU, OI, ht, NT, n, aj, nA, qN, eW, hw, Ak, h, aYf, dL, acd, Um, cC, oS, A, kj, aq, op, W, bP, mg, hR, bfX, fL, it, B, L, bL, fM, dA, cjn, ew, ON, C, V, q, J, qH, er, Kx, MR, WY, aT, jj, ae, bUd, wr, iU, Y, r, aaE, lo, bu, aaO, t, ck, rR, aIC, iQ, c, gX, bt, kh, pY, jh, oZ, dG, hF, mC, ad, Ae, oj, bj, ji, lQ, fb, nJ, uX, aCt, xs, eZ, app, akh, KM, bz, nb, bE, afd, Kj, bC, lC, KS, iu, y); #c1(NELFE) c2(XP_006715268) c3(negative elongation factor E isoform X1) c4(289147, 289148, 289149) c5(289150, 289151, 289152, 289153, 289154, 289155, 289156, 289157, 289158, 289159, 289160, 289161, 289162, 289163, 289164, 289165, 289166, 289167, 289168, 289169, 289170, 289171, 289172, 289173, 289174, 289175, 289176, 289177, 289178, 289179, 289180, 289181, 289182, 289183, 289184, 289185, 289186, 289187, 289188, 289189, 289190, 289191, 289192, 289193, 289194) c6( ) c7(T, os, aC, bB, gA, ci, x, aU, K, hu, H, eL, bc, aW, La, as, eE, cm, akw, aAW, eM, nB, iT, vA, U, dW, nw, k, m, avU, NK, gU, OI, ht, NT, n, aj, nA, qN, eW, hw, Ak, h, aYf, dL, acd, Um, cC, oS, A, kj, aq, op, W, bP, mg, hR, bfX, fL, it, B, L, bL, fM, dA, cjn, ew, ON, C, V, q, J, qH, er, Kx, MR, WY, aT, jj, ae, bUd, wr, iU, Y, r, aaE, lo, bu, aaO, t, ck, rR, aIC, iQ, c, gX, bt, kh, pY, jh, oZ, dG, hF, mC, ad, Ae, oj, bj, ji, lQ, fb, nJ, uX, aCt, xs, eZ, app, akh, KM, bz, nb, bE, afd, Kj, bC, lC, KS, iu, y); #c1(NELL1) c2(NP_001275643) c3(protein kinase C-binding protein NELL1 isoform 4 precursor) c4(106863) c5(106864, 106865, 106866, 106867, 106868, 106869) c6( ) c7(bM, T, pf, acl, bO, bS, ct, eA, yn, x, Mg, aE, gd, dL, K, an, rz, eL, A, Y, ev, ff, fe, agj, en, iV, Z, t, cm, fq, zV, bF, c, eM, ayS, m, k, UF, bt, ra); #c1(NELL1) c2(NP_006148) c3(protein kinase C-binding protein NELL1 isoform 1 precursor) c4(162412) c5(162413, 162414, 162415, 162416, 162417, 162418) c6( ) c7(bM, T, pf, acl, bO, bS, ct, eA, yn, x, Mg, aE, gd, dL, K, an, rz, eL, A, Y, ev, ff, fe, agj, en, iV, Z, t, cm, fq, zV, bF, c, eM, ayS, m, k, UF, bt, ra); #c1(NELL1) c2(NP_963845) c3(protein kinase C-binding protein NELL1 isoform 2 precursor) c4(262426) c5(262427, 262428, 262429, 262430, 262431, 262432) c6( ) c7(bM, T, pf, acl, bO, bS, ct, eA, yn, x, Mg, aE, gd, dL, K, an, rz, eL, A, Y, ev, ff, fe, agj, en, iV, Z, t, cm, fq, zV, bF, c, eM, ayS, m, k, UF, bt, ra); #c1(NELL2) c2(NP_001138579) c3(protein kinase C-binding protein NELL2 isoform a) c4(57042) c5(57043, 57044, 57045, 57046, 57047, 57048, 57049, 57050, 57051, 57052, 57053, 57054, 57055, 57056, 57057, 57058, 57059, 57060, 57061, 57062, 57063, 57064, 57065, 57066) c6(57067) c7(T, iT, eL, Y, eE, gX, apG, dL, EY, ci, K, m, bt, bz, q, yn, yR, UF); #c1(NELL2) c2(NP_001138580) c3(protein kinase C-binding protein NELL2 isoform b precursor) c4(57068) c5(57069, 57070, 57071, 57072, 57073, 57074, 57075, 57076, 57077, 57078, 57079, 57080, 57081, 57082, 57083, 57084, 57085, 57086, 57087) c6( ) c7(T, iT, eL, Y, eE, gX, apG, dL, EY, ci, K, m, bt, bz, q, yn, yR, UF); #c1(NELL2) c2(NP_001138581) c3(protein kinase C-binding protein NELL2 isoform c precursor) c4(57088, 57089, 57090) c5(57091, 57092, 57093, 57094, 57095, 57096, 57097, 57098, 57099, 57100, 57101, 57102, 57103, 57104, 57105, 57106, 57107, 57108, 57109, 57110, 57111, 57112, 57113, 57114, 57115, 57116, 57117, 57118, 57119, 57120) c6( ) c7(T, iT, eL, Y, eE, gX, apG, dL, EY, ci, K, m, bt, bz, q, yn, yR, UF); #c1(NELL2) c2(NP_001138582) c3(protein kinase C-binding protein NELL2 isoform d) c4(57121, 57122, 57123) c5(57124, 57125) c6( ) c7(T, iT, eL, Y, eE, gX, apG, dL, EY, ci, K, m, bt, bz, q, yn, yR, UF); #c1(NEMF) c2(NP_001288661) c3(nuclear export mediator factor NEMF isoform 2) c4(113913) c5(113914, 113915, 113916, 113917, 113918, 113919) c6( ) c7(bB, bE, bz, eM); #c1(NEMF) c2(NP_004704) c3(nuclear export mediator factor NEMF isoform 1) c4(148947, 148948) c5(148949, 148950, 148951, 148952, 148953, 148954, 148955, 148956, 148957, 148958, 148959, 148960) c6( ) c7(bB, bE, bz, eM); #c1(NEMF) c2(XP_005268258) c3(nuclear export mediator factor NEMF isoform X2) c4(281636) c5(281637, 281638, 281639, 281640, 281641, 281642, 281643, 281644, 281645, 281646, 281647, 281648) c6( ) c7(bB, bE, bz, eM); #c1(NEMF) c2(XP_011535618) c3(nuclear export mediator factor NEMF isoform X1) c4(332875) c5( ) c6( ) c7(bB, bE, bz, eM); #c1(NED1) c2(NP_001166094) c3(neogenin isoform 2 precursor) c4(68137) c5(68138, 68139, 68140, 68141, 68142, 68143) c6( ) c7(T, d, aE, iL, n, fD, Ea, t, it, eA); #c1(NEO1) c2(NP_001166095) c3(neogenin isoform 3 precursor) c4(68144) c5(68145, 68146, 68147, 68148, 68149, 68150) c6( ) c7(T, d, aE, iL, n, fD, Ea, t, it, eA); #c1(NEO1) c2(NP_002490) c3(neogenin isoform 1 precursor) c4(129269) c5(129270, 129271, 129272, 129273, 129274, 129275, 129276, 129277, 129278, 129279) c6( ) c7(T, d, aE, iL, n, fD, Ea, t, it, eA); #c1(NEO1) c2(XP_011519930) c3(neogenin isoform X1) c4( ) c5(314720) c6( ) c7(T, d, aE, iL, n, fD, Ea, t, it, eA); #c1(NEO1) c2(XP_011519933) c3(neogenin isoform X4) c4(314721) c5(314722, 314723, 314724, 314725, 314726, 314727) c6( ) c7(T, d, aE, iL, n, fD, Ea, t, it, eA); #c1(NES) c2(NP_006608) c3(nestin) c4(166588, 166589) c5(166590, 166591, 166592, 166593, 166594, 166595, 166596, 166597, 166598, 166599, 166600, 166601) c6( ) c7(Fv, T, aC, ea, if, fd, bv, aE, aeO, K, hm, eG, yo, aeR, aW, Ls, g, as, eF, cm, yX, eM, vA, fy, dD, aw, n, qN, cd, eW, b, ft, h, lG, SD, cY, A, aq, kp, RI, QK, op, dB, hR, va, fM, fZ, j, Cb, eA, RI, aT, cy, eu, jf, Uf, s, oV, Y, qL, en, cl, LV, dS, sk, wg, ac, iQ, aGi, dy, kG, hl, dG, uu, eq, iJ, ad, hP, fb, d, ff, eH, iR, bz, bF, amS, i, iu); #c1(NET1) c2(NP_001040625) c3(neuroepithelial cell-transforming gene 1 protein isoform 1) c4(34208, 34209) c5(34210, 34211, 34212, 34213, 34214, 34215, 34216) c6( ) c7(T, dB, aW, Jl, awa, AH, awb, aT, aA, aF, ci, K, g, B); #c1(NET1) c2(NP_005854) c3(neuroepithelial cell-transforming gene 1 protein isoform 2) c4(159916, 159917) c5(159918, 159919, 159920, 159921, 159922, 159923, 159924, 159925, 159926, 159927, 159928, 159929, 159930, 159931, 159932, 159933, 159934, 159935, 159936, 159937, 159938) c6( ) c7(T, dB, aW, Jl, awa, AH, awb, aT, aA, aF, ci, K, g, B); #c1(NET01) c2(NP_620552) c3(neuropilin and tolloid-like protein 1 isoform 1 precursor) c4(237709) c5(237710, 237711, 237712, 237713, 237714, 237715) c6( ) c7(T); #c1(NET01) c2(XP_005266831) c3(neuropilin and tolloid-like protein 1 isoform X1) c4(280338, 280339) c5(280340, 280341, 280342, 280343, 280344, 280345, 280346, 280347, 280348, 280349, 280350, 280351, 280352, 280353, 280354, 280355) c6( ) c7(T); #c1(NET02) c2(NP_060562) c3(neuropilin and tolloid-like protein 2 isoform 1 precursor) c4(196565) c5(196566, 196567, 196568, 196569, 196570, 196571, 196572, 196573, 196574, 196575) c6( ) c7(qK); #c1(NEU1) c2(NP_000425) c3(sialidase-1 precursor) c4(8817) c5(8818, 8819, 8820, 8821, 8822, 8823, 8824, 8825, 8826, 8827, 8828, 8829, 8830, 8831, 8832, 8833, 8834, 8835, 8836, 8837, 8838, 8839, 8840, 8841, 8842, 8843, 8844, 8845, 8846) c6( ) c7(T, id, aYP, n, dG, beZ, ci, fr, yU, AO, q, ad, eA, XX, Db, bzk, aT, yZ, ey, H, ae, eL, xQ, d, qt, aW, Ls, eH, eZ, aYR, aF, g, ys, cl, t, as, bz, eF, ir, kP, eM, aYa, aYb, aYQ, Lp, Zn, Lq, m, cjo, bnl, L); #c1(NEU3) c2(NP_006647) c3(sialidase-3) c4(166855, 166856, 166857) c5(166858, 166859, 166860, 166861, 166862, 166863, 166864, 166865, 166866, 166867, 166868, 166869, 166870, 166871, 166872, 166873, 166874, 166875, 166876, 166877, 166878, 166879) c6( ) c7(x, gh, T, aYa, bE, Y, bzk, bB, fr, ci, k, ae, X, ad); #c1(NEURL1) c2(NP_004201) c3(E3 ubiquitin-protein ligase NEURL1) c4(144303) c5(144304, 144305, 144306, 144307, 144308, 144309) c6( ) c7(T, id, n, fr, ci, AO, q, ad, eA, XX, Db, aT, ae, eL, jf, xQ, d, qt, aW, Ls, eH, aF, g, cl, as, t, eF, bz, ir, kP, eM, Zn, m, bnl); #c1(NEURL1) c2(XP_005270326) c3(E3 ubiquitin-protein ligase NEURL1 isoform X3) c4(283340, 283341, 283342) c5(283343, 283344, 283345, 283346, 283347, 283348, 283349, 283350, 283351, 283352, 283353, 283354) c6( ) c7(T, id, n, fr, ci, AO, q, ad, eA, XX, Db, aT, ae, eL, jf, xQ, d, qt, aW, Ls, eH, aF, g, cl, as, t, eF, bz, ir, kP, eM, Zn, m, bnl); #c1(NEURL1) c2(XP_011538637) c3(E3 ubiquitin-protein ligase NEURL1 isoform X6) c4( ) c5(336379, 336380, 336381, 336382, 336383, 336384) c6( ) c7(T, id, n, fr, ci, AO, q, ad, eA, XX, Db, aT, ae, eL, jf, xQ, d, qt, aW, Ls, CH, aF, g, cl, as, t, eF, bz, ir, kP, eM, Zn, m, bnl); #c1(NEURL2) c2(NP_542787) c3(neutralized-like protein 2 isoform 1) c4(233093) c5(233094, 233095, 233096, 233097, 233098, 233099) c6( ) c7(km); #c1(NEUROD1) c2(NP_002491) c3(neurogenic differentiation factor 1) c4(129280) c5(129281, 129282, 129283, 129284, 129285, 129286, (29287) c6( ) c7(bM, T, byb, e, ame, ci, aQK, aR, x, aE, pX, vg, qM, Us, hm, yo, aW, hl, g, cm, qJ, y, bhB, eM, iA, Gs, mx, vy, cj, uJ, OD, m, pw, zv, iu, cZ, dt, cV, aj, fr, cd, ai, xC, Mo, ft, mh, i, BR, ach, cjq, cU, dd, A, Cw, sp, ahJ, agF, aPP, CQ, jT, mg, hR, ER, art, aNX, B, aNW, bL, Dw, Rt, cz, tc, QZ, v, V, q, J, eA, oT, lg, aT, bK, jj, bq, Cy, ae, jf, arl, s, xR, oV, Y, cjp, r, lo, t, arJ, pb, ck, cT, BU, ac, lV, mG, nG, wR, mf, dy, bt, hS, pY, iW, jh, dG, aNH, hF, ad, av, tT, oW, hD, agm, lQ, sj, mu, d, lx, Gv, xs, cr, dN, S, bz, qa, blZ, bF, es, eL, qe, bE, z, ag, dX, pk, lf, gq, hv); #c1(NEUROD2) c2(XP_005257466) c3(neurogenic differentiation factor 2 isoform X2) c4( ) c5(275136, 275137) c6( ) c7(jf, A, pz); #c1(NEUROD2) c2(XP_011523153) c3(neurogenic differentiation factor 2 isoform X1) c4(317688) c5(317689, 317690, 317691, 317692, 317693) c6( ) c7(jf, A, pz); #c1(NEUROD4) c2(NP_067014) c3(neurogenic differentiation factor 4) c4(208358) c5(208359, 208360, 208361, 208362, 208363, 208364, 208365) c6( ) c7(bL, aj, J); #c1(NEUROD6) c2(NP_073565) c3(neurogenic differentiation factor 6) c4(212867) c5(212868, 212869, 212870, 212871, 212872, 212873, 212874) c6( ) c7(aXL); #c1(NEUROG1) c2(NP_006152) c3(neurogenin-1) c4(162419) c5(162420, 162421, 162422, 162423, 162424, 162425, 162426) c6( ) c7(jf, Mb, T, yo, jz, bP, c, s, iH, ajN, iI, r, n, ci, q, bN); #c1(NEUROG2) c2(NP_076924) c3(neurogenin-2) c4(214268) c5(214269, 214270, 214271, 214272, 214273, 214274, 214275, 214276, 214277, 214278) c6( ) c7(yo, s, an); #c1(NEUROG3) c2(NP_066279) c3(neurogenin-3) c4(206954) c5(206955, 206956, 206957, 206958, 206959, 206960, 206961, 206962) c6( ) c7(bL, yo, A, mh, pY, cjr, Hm, UG, ahJ, aj, mf, j, J); #c1(NF1) c2(NP_000258) c3(neurofibromin isoform 2) c4(5861) c5(5862, 5863, 5864, 5865, 5866, 5867) c6( ) c7(T, aC, yS, e, bB, amj, ci, ip, bv, cjw, aeO, Qk, ajv, Ev, Ou, K, aBK, eG, aeR, aW, Ls, Az, uY, as, cm, bw, iP, lq, QF, m, adk, n, nA, Cd, b, ft, bkm, TK, Dz, dx, cC, aVf, iw, blP, atf, Hu, cjt, iN, byl, bkn, dB, anx, bD, agx, yO, eA, XX, nX, aT, dz, jf, qw, iU, Y, en, bQ, f, bN, ck, QG, acW, Dy, au, dh, aeM, cjv, bt, xb, VB, jh, agz, hF, xt, iC, ib, cjx, hD, bma, hP, fb, pq, cjs, iL, d, ff, cr, kD, aiz, VI, bz, dr, dR, aHY, Hp, cju, Ew, bE, gZ, aS, i, xg, jr); #c1(NF1) c2(NP_001035957) c3(neurofibromin isoform 1) c4(33538) c5(33539, 33540, 33541, 33542, 33543, 33544) c6( ) c7(T, aC, yS, e, bB, amj, ci, ip, bv, cjw, aeO, Qk, ajv, Ev, Ou, K, aBK, eG, aeR, aW, Ls, Az, uY, as, cm, bw, iP, lq, QF, m, adk, n, nA, Cd, b, ft, bkm, TK, Dz, dx, cC, aVf, iw, blP, atf, Hu, cjt, iN, byl, bkn, dB, anx, bD, agx, yO, eA, XX, nX, aT, dz, jf, qw, iU, Y, en, bQ, f, bN, ck, QG, acW, Dy, au, dh, aeM, cjv, bt, xb, VB, jh, agz, hF, xt, iC, ib, cjx, hD, bma, hP, fb, pq, cjs, iL, d, ff, cr, kD, aiz, VI, bz, dr, dR, aHY, Hp, cju, Ew, bE, gZ, aS, i, xg, jr); #c1(NF1) c2(NP_001121619) c3(neurofibromin isoform 3) c4(45878) c5(45879, 45880, 45881, 45882, 45883, 45884) c6( ) c7(T, aC, yS, e, bB, amj, ci, ip, bv, cjw, aeO, Qk, ajv, Ev, Ou, K, aBK, eG, aeR, aW, Ls, Az, uY, as, cm, bw, iP, lq, QF, m, adk, n, nA, Cd, b, ft, bkm, TK, Dz, dx, cC, aVf, iw, blP, atf, Hu, cjt, iN, byl, bkn, dB, anx, bD, agx, yO, eA, XX, nX, aT, dz, jf, qw, iU, Y, en, bQ, f, bN, ck, QG, acW, Dy, au, dh, aeM, cjv, bt, xb, VB, jh, agz, hF, xt, iC, ib, cjx, hD, bma, hP, fb, pq, cjs, iL, d, ff, cr, kD, aiz, VI, bz, dr, dR, aHY, Hp, cju, Ew, bE, gZ, aS, i, xg, jr); #c1(NF1) c2(XP_005258040) c3(neurofibromin isoform X3) c4(275464) c5( ) c6( ) c7(T, aC, yS, e, bB, amj, ci, ip, bv, cjw, aeO, Qk, ajv, Ev, Ou, K, aBK, eG, aeR, aW, Ls, Az, uY, as, cm, bw, iP, lq, QF, m, adk, n, nA, Cd, b, ft, bkm, TK, Dz, dx, cC, aVf, iw, blP, atf, Hu, cjt, iN, byl, bkn, dB, anx, bD, agx, yO, eA, XX, nX, aT, dz, jf, qw, iU, Y, en, bQ, f, bN, ck, QG, acW, Dy, au, dh, aeM, cjv, bt, xb, VB, jh, agz, hF, xt, iC, ib, cjx, hD, bma, hP, fb, pq, cjs, iL, d, ff, cr, kD, aiz, VI, bz, dr, dR, aHY, Hp, cju, Ew, bE, gZ, aS, i, xg, jr); #c1(NF1) c2(XP_005258041) c3(neurofibromin isoform X7) c4(275465) c5(275466, 275467, 275468, 275469, 275470, 275471) c6( ) c7(T, aC, yS, e, bB, amj, ci, ip, bv, cjw, aeO, Qk, ajv, Ev, Ou, K, aBK, eG, aeR, aW, Ls, Az, uY, as, cm, bw, iP, lq, QF, m, adk, n, nA, Cd, b, ft, bkm, TK, Dz, dx, cC, aVf, iw, blP, atf, Hu, cjt, iN, byl, bkn, dB, anx, bD, agx, yO, eA, XX, nX, aT, dz, jf, qw, iU, Y, en, bQ, f, bN, ck, QG, acW, Dy, au, dh, aeM, cjv, bt, xb, VB, jh, agz, hF, xt, iC, ib, cjx, hD, bma, hP, fb, pq, cjs, iL, d, ff, cr, kD, aiz, VI, bz, dr, dR, aHY, Hp, cju, Ew, bE, gZ, aS, i, xg, jr); #c1(NF1) c2(XP_006721985) c3(neurofibromin isoform X1) c4( ) c5(294518, 294519, 294520, 294521, 294522) c6( ) c7(T, aC, yS, e, bB, amj, ci, ip, bv, cjw, aeO, Qk, ajv, Ev, Ou, K, aBK, eG, aeR, aW, Ls, Az, uY, as, cm, bw, iP, lq, QF, m, adk, n, nA, Cd, b, ft, bkm, TK, Dz, dx, cC, aVf, iw, blP, atf, Hu, cjt, iN, byl, bkn, dB, anx, bD, agx, yO, eA, XX, nX, aT, dz, jf, qw, iU, Y, en, bQ, f, bN, ck, QG, acW, Dy, au, dh, aeM, cjv, bt, xb, VB, jh, agz, hF, xt, iC, ib, cjx, hD, bma, hP, fb, pq, cjs, iL, d, ff, cr, kD, aiz, VI, bz, dr, dR, aHY, Hp, cju, Ew, bE, gZ, aS, i, xg, jr); #c1(NF1) c2(XP_006721987) c3(neurofibromin isoform X5) c4(294523) c5(294524, 294525, 294526, 294527, 294528, 294529) c6( ) c7(T, aC, yS, e, bB, amj, ci, ip, bv, cjw, aeO, Qk, ajv, Ev, Ou, K, aBK, eG, aeR, aW, Ls, Az, uY, as, cm, bw, iP, lq, QF, m, adk, n, nA, Cd, b, ft, bkm, TK, Dz, dx, cC, aVf, iw, blP, atf, Hu, cjt, iN, byl, bkn, dB, anx, bD, agx, yO, eA, XX, nX, aT, dz, jf, qw, iU, Y, en, bQ, f, bN, ck, QG, acW, Dy, au, dh, aeM, cjv, bt, xb, VB, jh, agz, hF, xt, iC, ib, cjx, hD, bma, hP, fb, pq, cjs, iL, d, ff, cr, kD, aiz, VI, bz, dr, dR, aHY, Hp, cju, Ew, bE, gZ, aS, i, xg, jr); #c1(NF1) c2(XP_011523159) c3(neurofibromin isoform X8) c4( ) c5(317694) c6( ) c7(T, aC, yS, e, bB, amj, ci, ip, bv, cjw, aeO, Qk, ajv, Ev, Ou, K, aBK, eG, aeR, aW, Ls, Az, uY, as, cm, bw, iP, lq, QF, m, adk, n, nA, Cd, b, ft, bkm, TK, Dz, dx, cC, aVf, iw, blP, atf, Hu, cjt, iN, byl, bkn, dB, anx, bD, agx, yO, eA, XX, nX, aT, dz, jf, qw, iU, Y, en, bQ, f, bN, ck, QG, acW, Dy, au, dh, aeM, cjv, bt, xb, VB, jh, agz, hF, xt, iC, ib, cjx, hD, bma, hP, fb, pq, cjs, iL, d, ff, cr, kD, aiz, VI, bz, dr, dR, aHY, Hp, cju, Ew, bE, gZ, aS, i, xg, jr); #c1(NF2) c2(NP_000259) c3(merlin isoform 1) c4(5868, 5869) c5(5870, 5871, 5872, 5873, 5874, 5875) c6( ) c7(agx, cZ, T, aC, ob, ha, jx, aA, n, KO, Zh, fr, agz, hF, anu, q, ad, qH, cjy, eA, ip, iC, ib, avs, aeO, Qk, aT, hD, h, By, bkw, dL, dd, cfu, fb, eL, dx, Fp, Uf, d, aeR, aW, Y, oV, Ls, r, gl, uY, aq, aF, as, eF, cjz, kJ, bz, ayg, cm, ck, pH, Ew, eM, aBU, dB, hk, aJQ, avr, azr, bt, B, hS); #c1(NF2) c2(NP_861546) c3(merlin isoform 2) c4(255671, 255672) c5(255673, 255674, 255675, 255676, 255677, 255678, 255679, 255680, 255681, 255682, 255683, 255684, 255685, 255686, 255687, 255688, 255689, 255690, 255691, 255692, 255693, 255694, 255695, 255696, 255697, 255698) c6( ) c7(agx, cZ, T, aC, ob, ha, jx, aA, n, KO, Zh, fr, agz, hF, anu, q, ad, qH, cjy, eA, ip, iC, ib, avs, aeO, Qk, aT, hD, h, By, bkw, dL, dd, cfu, fb, eL, dx, Fp, Uf, d, aeR, aW, Y, oV, Ls, r, gl, uY, aq, aF, as, eF, cjz, kJ, bz, ayg, cm, ck, pH, Ew, eM, aBU, dB, hk, aJQ, avr, azr, bt, B, hS); #c1(NF2) c2(NP_861966) c3(merlin isoform 5) c4(255699, 255700) c5(255701, 255702, 255703, 255704, 255705, 255706) c6( ) c7(agx, cZ, T, aC, ob, ha, jx, aA, n, KO, Zh, fr, agz, hF, anu, q, ad, qH, cjy, eA, ip, iC, ib, avs, aeO, Qk, aT, hD, h, By, bkw, dL, dd, cfu, fb, eL, dx, Fp, Uf, d, aeR, aW, Y, oV, Ls, r, gl, uY, aq, aF, as, eF, cjz, kJ, bz, ayg, cm, ck, pH, Ew, eM, aBU, dB, hk, aJQ, avr, azr, bt, B, hS); #c1(NF2) c2(NP_861967) c3(merlin isoform 6) c4(255707) c5(255708, 255709, 255710, 255711, 255712, 255713) c6( ) c7(agx, cZ, T, aC, ob, ha, jx, aA, n, KO, Zh, fr, agz, hF, anu, q, ad, qH, cjy, eA, ip, iC, ib, avs, aeO, Qk, aT, hD, h, By, bkw, dL, dd, cfu, fb, eL, dx, Fp, Uf, d, aeR, aW, Y, oV, Ls, r, gl, uY, aq, aF, as, eF, cjz, kJ, bz, ayg, cm, ck, pH, Ew, eM, aBU, dB, hk, aJQ, avr, azr, bt, B, hS); #c1(NF2) c2(NP_861969) c3(merlin isoform 7) c4(255714) c5(255715, 255716, 255717, 255718, 255719, 255720, 255721, 255722, 255723, 255724, 255725, 255726, 255727, 255728, 255729, 255730, 255731, 255732, 255733, 255734) c6( ) c7(agx, cZ, T, aC, ob, ha, jx, aA, n, KO, Zh, fr, agz, hF, anu, q, ad, qH, cjy, eA, ip, iC, ib, avs, aeO, Qk, aT, hD, h, By, bkw, dL, dd, cfu, fb, eL, dx, Fp, Uf, d, aeR, aW, Y, oV, Ls, r, gl, uY, aq, aF, as, eF, cjz, kJ, bz, ayg, cm, ck, pH, Ew, eM, aBU, dB, hk, aJQ, avr, azr, bt, B, hS); #c1(NF2) c2(NP_861971) c3(merlin isoform 8) c4(255735) c5(255736, 255737, 255738, 255739, 255740, 255741) c6( ) c7(agx, cZ, T, aC, ob, ha, jx, aA, n, KO, Zh, fr, agz, hF, anu, q, ad, qH, cjy, eA, ip, iC, ib, avs, aeO, Qk, aT, hD, h, By, bkw, dL, dd, cfu, fb, eL, dx, Fp, Uf, d, aeR, aW, Y, oV, Ls, r, gl, uY, aq, aF, as, eF, cjz, kJ, bz, ayg, cm, ck, pH, Ew, eM, aBU, dB, hk, aJQ, avr, azr, bt, B, hS); #c1(NFAM1) c2(NP_666017) c3(NFAT activation molecule 1 precursor) c4(241998) c5( ) c6( ) c7(FR); #c1(NFAM1) c2(XP_011528259) c3(NFAT activation molecule 1 isoform X1) c4( ) c5(323342, 323343, 323344, 323345, 323346, 323347) c6( ) c7(fR); #c1(NFASC) c2(NP_001005388) c3(neurofascin isoform 1 precursor) c4(21613) c5(21614, 21615, 21616, 21617, 21618, 21619, 21620, 21621, 21622, 21623) c6( ) c7(ans, mF, V, yW); #c1(NFASC) c2(NP_001005389) c3(neurofascin isoform 5 precursor) c4(21624) c5(21625, 21626, 21627, 21628, 21629, 21630, 21631, 21632, 21633, 21634, 21635, 21636, 21637, 21638, 21639) c6( ) c7(ans, mF, V, yW); #c1(NFASC) c2(NP_055905) c3(neurofascin isoform 4 precursor) c4( ) c5(184381, 184382, 184383, 184384, 184385, 184386, 184387, 184388) c6( ) c7(ans, mF, V, yW); #c1(NFASC) c2(XP_005245046) c3(neurofascin isoform X16) c4(266227, 266228) c5(266229, 266230, 266231, 266232, 266233, 266234, 266235, 266236, 266237, 266238, 266239, 266240, 266241) c6( ) c7(ans, mF, V, yW); #c1(NFASC) c2(XP_005245048) c3(neurofascin isoform X17) c4(266242) c5(266243, 266244, 266245, 266246, 266247, 266248, 266249, 266250, 266251, 266252) c6( ) c7(ans, mF, V, yW); #c1(NFASC) c2(XP_005245049) c3(neurofascin isoform X18) c4(266253) c5(266254, 266255) c6( ) c7(ans, mF, V, yW); #c1(NFASC) c2(XP_005245054) c3(neurofascin isoform X22) c4( ) c5(266256, 266257) c6( ) c7(ans, mF, V, yW); #c1(NFASC) c2(XP_011507617) c3(neurofascin isoform X5) c4(297524) c5(297525, 297526, 297527, 297528, 297529) c6( ) c7(ans, mF, V, yW); #c1(NFASC) c2(XP_011507621) c3(neurofascin isoform X9) c4(297530, 297531, 297532) c5(297533, 297534, 297535, 297536, 297537) c6( ) c7(ans, mF, V, yW); #c1(NFASC) c2(XP_011507622) c3 (neurofascin isoform X10) c4( ) c5(297538, 297539, 297540) c6( ) c7(ans, mF, V, yW); #c1(NFASC) c2(XP_011507623) c3(neurofascin isoform X11) c4(297541, 297542) c5(297543, 297544, 297545, 297546, 297547, 297548, 297549, 297550, 297551, 297552, 297553) c6( ) c7(ans, mF, V, yW); #c1(NFASC) c2(XP_011507624) c3 (neurofascin isoform X12) c4(297554) c5(297555, 297556, 297557, 297558, 297559, 297560) c6( ) c7(ans, mF, V, yW); #c1(NFASC) c2(XP_011507630) c3(neurofascin isoform X23) c4(297561) c5(297562, 297563, 297564, 297565, 297566, 297567) c6( ) c7(ans, mF, V, yW); #c1(NFAT5) c2(NP_001106649) c3(nuclear factor of activated T-cells 5 isoform d) c4(42451) c5(42452, 42453, 42454, 42455, 42456, 42457, 42458, 42459, 42460, 42461) c6( ) c7(ck, T, cF, op, aW, vA, vy, aT, cz, cy, hR, dG, h, K, y, ai, Pm); #c1(NFAT5) c2(NP_006590) c3(nuclear factor of activated T-cells 5 isoform c) c4(166489, 166490) c5(166491, 166492, 166493, 166494, 166495, 166496, 166497) c6( ) c7(ck, T, cF, op, aW, vA, vy, aT, cz, cy, hR, dG, h, K, y, ai, Pm); #c1(NFAT5) c2(NP_619727) c3(nuclear factor of activated T-cells 5 isoform b) c4(237050) c5(237051, 237052, 237053, 237054, 237055, 237056) c6( ) c7(ck, T, cF, op, aW, vA, vy, aT, cz, cy, hR, dG, h, K, y, ai, Pm); #c1(NFAT5) c2(NP_619728) c3(nuclear factor of activated T-cells 5 isoform a) c4( ) c5(237057, 237058, 237059, 237060, 237061, 237062, 237063, 237064, 237065, 237066, 237067, 237068, 237069, 237070) c6( ) c7(ck, T, cF, op, aW, vA, vy, aT, cz, cy, hR, dG, h, K, y, ai, Pm); #c1(NFATC1) c2(NP_001265598) c3(nuclear factor of activated T-cells, cytoplasmic 1 isoform F) c4(98930, 98931, 98932) c5(98933, 98934, 98935, 98936, 98937, 98938, 98939, 98940, 98941, 98942, 98943, 98944, 98945, 98946, 98947, 98948, 98949, 98950, 98951, 98952, 98953, 98954, 98955, 98956, 98957) c6( ) c7(T, aC, acl, oe, jh, cjA, j, ci, Ea, ai, aE, aT, h, pi, cp, lC, cr, akB, BG, iV, f, t, lY, pa, aLw, bE, cj, gZ, y, kS); #c1(NFATC1) c2(NP_001265599) c3(nuclear factor of activated T-cells, cytoplasmic 1 isoform G) c4(98958) c5(98959, 98960, 98961, 98962, 98963, 98964) c6( ) c7(T, aC, acl, oe, jh, cjA, j, ci, Ea, ai, aE, aT, h, pi, cp, lC, cr, akB, BG, iV, f, t, lY, pa, aLw, bE, cj, gZ, y, kS); #c1(NFATC1) c2(NP_001265602) c3(nuclear factor of activated T-cells, cytoplasmic 1 isoform J) c4( ) c5(98965, 98966, 98967, 98968, 98969, 98970, 98971, 98972) c6( ) c7(T, aC, acl, oe, jh, cjA, j, ci, Ea, ai, aE, aT, h, pi, cp, lC, cr, akB, BG, iV, f, t, lY, pa, aLw, bE, cj, gZ, y, kS); #c1(NFATC1) c2(NP_006153) c3(nuclear factor of activated T-cells, cytoplasmic 1 isoform B) c4(162427, 162428) c5( ) c6( ) c7(T, aC, acl, oe, jh, cjA, j, ci, Ea, ai, aE, aT, h, pi, cp, lC, cr, akB, BG, iV, f, t, lY, pa, aLw, bE, cj, gZ, y, kS); #c1(NFATC1) c2(NP_765975) c3(nuclear factor of activated T-cells, cytoplasmic 1 isoform C) c4(249440, 249441) c5(249442, 249443, 249444, 249445, 249446, 249447, 249448, 249449, 249450, 249451, 249452, 249453) c6( ) c7(T, aC, acl, oe, jh, cjA, j, ci, Ea, ai, aE, aT, h, pi, cp, lC, cr, akB, BG, iV, f, t, lY, pa, aLw, bE, cj, gZ, y, kS); #c1(NFATC1) c2(NP_765976) c3(nuclear factor of activated T-cells, cytoplasmic 1 isoform D) c4( ) c5(249454, 249455, 249456, 249457, 249458) c6( ) c7(T, aC, acl, oe, jh, cjA, j, ci, Ea, ai, aE, aT, h, pi, cp, lC, cr, akB, BG, iV, f, t, lY, pa, aLw, bE, cj, gZ, y, kS); #c1(NFATC1) c2(NP_765977) c3(nuclear factor of activated T-cells, cytoplasmic 1 isoform E) c4( ) c5(249459) c6( ) c7(T, aC, acl, oe, jh, cjA, j, ci, Ea, ai, aE, aT, h, pi, cp, lC, cr, akB, BG, iV, f, t, lY, pa, aLw, bE, cj, gZ, y, kS); #c1(NFATC1) c2(NP_765978) c3(nuclear factor of activated T-cells, cytoplasmic 1 isoform A) c4(249460) c5(249461, 249462, 249463, 249464, 249465, 249466) c6( ) c7(T, aC, acl, oe, jh, cjA, j, ci, Ea, ai, aE, aT, h, pi, cp, lC, cr, akB, BG, iV, f, t, lY, pa, aLw, bE, cj, gZ, y, kS); #c1(NFATC2) c2(NP_001129493) c3(nuclear factor of activated T-cells, cytoplasmic 2 isoform D) c4(51638) c5(51639, 51640, 51641, 51642, 51643, 51644, 51645) c6( ) c7(bL, lD, T, kh, jh, bB, aj, v, ci, q, aR, J, cd, eA, av, cF, aE, sO, abq, cjB, aT, h, ji, pk, dx, cp, uQ, ly, Nr, aW, Y, po, BG, iV, Z, LV, f, lx, Nq, ck, pc, bE, oy, akC, dW, fD, lC, ra, B); #c1(NFATC2) c2(NP_001245225) c3 (nuclear factor of activated T-cells, cytoplasmic 2 isoform F) c4( ) c5(92751, 92752, 92753, 92754, 92755, 92756) c6( ) c7(bL, lD, T, kh, jh, bB, aj, v, ci, q, aR, J, cd, eA, av, cF, aE, sO, abq, cjB, aT, h, ji, pk, dx, cp, uQ, ly, Nr, aW, Y, po, BG, iV, Z, LV, f, lx, Nq, ck, pc, bE, oy, akC, dW, fD, lC, ra, B); #c1(NFATC2) c2(NP_001245226) c3(nuclear factor of activated T-cells, cytoplasmic 2 isoform G) c4(92757) c5( ) c6( ) c7(bL, lD, T, kh, jh, bB, aj, v, ci, q, aR, J, cd, eA, av, cF, aE, sO, abq, cjB, aT, h, ji, pk, dx, cp, uQ, ly, Nr, aW, Y, po, BG, iV, Z, LV, f, lx, Nq, ck, pc, bE, oy, akC, dW, fD, lC, ra, B); #c1(NFATC2) c2(NP_036472) c3(nuclear factor of activated T-cells, cytoplasmic 2 isoform B) c4(174095) c5(174096, 174097, 174098, 174099, 174100, 174101, 174102) c6( ) c7(bL, lD, T, kh, jh, bB, aj, v, ci, q, aR, J, cd, eA, av, cF, aE, sO, abq, cjB, aT, h, ji, pk, dx, cp, uQ, ly, Nr, aW, Y, po, BG, iV, Z, LV, f, lx, Nq, ck, pc, bE, oy, akC, dW, fD, lC, ra, B); #c1(NFATC2) c2(NP_775114) c3(nuclear factor of activated T-cells, cytoplasmic 2 isoform C) c4(249632) c5(249633, 249634, 249635, 249636, 249637, 249638) c6( ) c7(bL, lD, T, kh, jh, bB, aj, v, ci, q, aR, J, cd, eA, av, cF, aE, sO, abq, cjB, aT, h, ji, pk, dx, cp, uQ, ly, Nr, aW, Y, po, BG, iV, Z, LV, f, lx, Nq, ck, pc, bE, oy, akC, dW, fD, lC, ra, B); #c1(NFATC3) c2(NP_004546) c3(nuclear factor of activated T-cells, cytoplasmic 3 isoform 2) c4(147477, 147478) c5(147479, 147480, 147481, 147482, 147483, 147484, 147485, 147486, 147487, 147488, 147489, 147490, 147491) c6( ) c7(ck, T, fR, aW, gX, aT, lo, aj, lC, J, ad); #c1(NFATC3) c2(NP_775186) c3(nuclear factor of activated T-cells, cytoplasmic 3 isoform 3) c4(249664) c5(249665, 249666, 249667, 249668, 249669, 249670, 249671, 249672) c6( ) c7(ck, T, fR, aW, gX, aT, lo, aj, lC, J, ad); #c1(NFATC3) c2(NP_775188) c3(nuclear factor of activated T-cells, cytoplasmic 3 isoform 1) c4(249673) c5(249674, 249675, 249676, 249677, 249678, 249679) c6( ) c7(ck, T, fR, aW, gX, aT, lo, aj, lC, J, ad); #c1(NFATC4) c2(NP_001129494) c3(nuclear factor of activated T-cells, cytoplasmic 4 isoform 1) c4(51646, 51647, 51648, 51649, 51650, 51651, 51652) c5(51653, 51654, 51655, 51656, 51657, 51658, 51659, 51660, 51661, 51662, 51663, 51664, 51665, 51666, 51667, 51668, 51669, 51670, 51671, 51672, 51673, 51674, 51675, 51676, 51677, 51678, 51679, 51680, 51681, 51682, 51683, 51684) c6( ) c7(cc, af, aW, sO, abq, mx, aT, aj, v, yO, cd, J); #c1(NFATC4) c2(NP_001185894) c3(nuclear factor of activated T-cells, cytoplasmic 4 isoform 3) c4( ) c5(76680, 76681, 76682, 76683, 76684, 76685, 76686, 76687, 76688, 76689) c6( ) c7(cc, af, aW, sO, abq, mx, al, aj, v, yO, cd, J); #c1(NFATC4) c2(NP_001185895) c3(nuclear factor of activated T-cells, cytoplasmic 4 isoform 4) c4(76690, 76691, 76692) c5(76693, 76694, 76695, 76696, 76697, 76698, 76699, 76700, 76701, 76702, 76703, 76704) c6( ) c7(cc, af, aW, sO, abq, mx, aT, aj, v, yO, cd, J); #c1(NFATC4) c2(NP_001185896) c3(nuclear factor of activated T-cells, cytoplasmic 4 isoform 5) c4(76705, 76706, 76707, 76708, 76709, 76710, 76711, 76712) c5(76713, 76714, 76715, 76716, 76717, 76718, 76719, 76720, 76721, 76722, 76723, 76724, 76725, 76726, 76727, 76728, 76729, 76730, 76731, 76732, 76733, 76734, 76735, 76736, 76737) c6( ) c7(cc, af, aW, sO, abq, mx, aT, aj, v, yO, cd, J); #c1(NFATC4) c2(NP_001275731) c3(nuclear factor of activated T-cells, cytoplasmic 4 isoform 6) c4(107198) c5(107199, 107200, 107201, 107202, 107203, 107204, 107205, 107206, 107207, 107208, 107209, 107210, 107211, 107212, 107213, 107214, 107215, 107216, 107217, 107218, 107219, 107220, 107221, 107222) c6( ) c7(cc, af, aW, sO, abq, mx, aT, aj, v, yO, cd, J); #c1(NFATC4) c2(NP_004545) c3(nuclear factor of activated T-cells, cytoplasmic 4 isoform 2) c4(147463, 147464, 147465) c5(147466, 147467, 147468, 147469, 147470, 147471, 147472, 147473, 147474, 147475, 147476) c6( ) c7(cc, af, aW, sO, abq, mx, aT, aj, v, yO, cd, J); #c1(NFATC4) c2(XP_011535099) c3(nuclear factor of activated T-cells, cytoplasmic 4 isoform X1) c4(331848) c5(331849, 331850, 331851, 331852, 331853, 331854) c6( ) c7(cc, af, aW, sO, abq, mx, aT, aj, v, yO, cd, J); #c1(NFATC4) c2(XP_011535100) c3(nuclear factor of activated T-cells, cytoplasmic 4 isoform X2) c4( ) c5(331855, 331856, 331857, 331858, 331859, 331860, 331861, 331862, 331863, 331864) c6( ) c7(cc, af, aW, sO, abq, mx, aT, aj, v, yO, cd, J); #c1(NFATC4) c2(XP_011535101) c3(nuclear factor of activated T-cells, cytoplasmic 4 isoform X3) c4(331865) c5(331866, 331867, 331868, 331869, 331870, 331871, 331872, 331873, 331874, 331875, 331876, 331877, 331878, 331879, 331880, 331881, 331882, 331883, 331884, 331885, 331886, 331887, 331888, 331889, 331890, 331891) c6( ) c7(cc, af, aW, sO, abq, mx, aT, aj, v, yO, cd, J); #c1(NFE2) c2(XP_005268963) c3 (transcription factor NF-E2 45 kDa subunit isoform X1) c4(282226, 282227) c5(282228, 282229, 282230, 282231, 282232, 282233, 282234, 282235, 282236, 282237, 282238, 282239, 282240, 282241, 282242, 282243, 282244, 282245, 282246, 282247, 282248, 282249, 282250, 282251, 282252) c6( ) c7(T, dD, ea, qj, eq, C, oT, oW, aT, dz, K, zy, et, aW, kD, pm, age, pc, eM, pg, XO, pC); #c1(NFE2L1) c2(NP_003195) c3(nuclear factor erythroid 2-related factor 1) c4(136179, 136180) c5(136181, 136182, 136183, 136184, 136185, 136186, 136187, 136188, 136189, 136190, 136191, 136192) c6( ) c7(bL, ae, kj, aW, aT, cd, aj, aq, bl, m, K, Ku, cm, fq, B, J, ad); #c1(NFE2L1) c2(XP_005257470) c3(nuclear factor erythroid 2-related factor 1 isoform X3) c4(275138) c5(275139, 275140, 275141, 275142, 275143, 275144, 275145, 275146, 275147, 275148, 275149, 275150) c6( ) c7(bL, ae, kj, aW, aT, cd, aj, aq, bl, m, K, Ku, cm, fq, B, J, ad); #c1(NFE2L1) c2(XP_005257472) c3(nuclear factor erythroid 2-related factor 1 isoform X4) c4(275151) c5( ) c6( ) c7(bL, ae, kj, aW, aT, cd, aj, aq, bl, m, K, Ku, cm, fq, B, J, ad); #c1(NFE2L2) c2(NP_001138884) c3(nuclear factor erythroid 2-related factor 2 isoform 2) c4(58055, 58056) c5(58057, 58058, 58059, 58060, 58061, 58062, 58063, 58064, 58065, 58066, 58067, 58068, 58069, 58070, 58071) c6( ) c7(oO, bM, T, aC, fV, ea, e, al, if, bB, qj, aY, fw, s, aR, bv, aE, dV, K, aDZ, eL, bc, N, Zm, aa, LC, aW, fc, hl, nY, Qq, aB, g, hJ, cm, fT, eM, nB, Kv, U, cj, NI, vy, tG, wx, Hb, m, bi, iu, FW, gU, dD, JY, aWG, n, BH, aj, asF, uE, PJ, qN, rP, Hx, ft, cc, bud, cU, lG, h, dL, hB, db, Nr, GZ, fJ, fQ, sm, aq, po, cM, adz, Ye, iO, W, dB, hk, Fi, Qu, hL, at, E, eo, cB, B, it, bl, eR, fG, hx, yk, aA, cR, fF, v, j, C, il, V, q, J, er, eA, Wu, aT, cy, jj, an, ae, af, dl, Lu, s, oV, Y, r, lo, en, cl, t, iK, ck, sk, rR, nM, ac, c, kB, dh, dy, fD, bt, KG, ao, am, qb, aD, ha, dG, eq, ad, hX, fU, ceQ, hd, ib, aPn, av, byL, oz, oW, io, ji, lQ, d, lR, ff, eZ, dq, gE, aF, byM, aot, iR, iV, KM, bz, ek, iq, Nq, LB, D, xZ, bE, aS, cP, lC, tP, kS, X, jr, BA, pC); #c1(NFE2L2) c2(NP_001138885) c3(nuclear factor erythroid 2-related factor 2 isoform 3) c4(58072) c5(58073, 58074, 58075, 58076, 58077, 58078) c6( ) c7(oO, bM, T, aC, fV, ea, e, al, if, bB, qj, aY, fw, sD, aR, bv, aE, dV, K, aDZ, eL, bc, N, Zm, aa, LC, aW, fc, hl, nY, Qq, aB, g, hJ, cm, fT, eM, nB, Kv, U, cj, NI, vy, tG, wx, Hb, m, bi, iu, FW, gU, dD, JY, aWG, n, BH, aj, asF, uE, PJ, qN, rP, Hx, ft, cc, bud, cU, lG, h, dL, hB, db, Nr, GZ, fJ, fQ, sm, aq, po, cM, adz, Ye, iO, W, dB, hk, Fi, Qu, hL, at, E, eo, cB, B, it, bl, eR, fG, hx, yk, aA, cR, fF, v, j, C, iI, V, q, J, er, eA, Wu, aT, cy, jj, an, ae, af, dl, Lu, s, oV, Y, r, lo, en, cl, t, iK, ck, sk, rR, nM, ac, c, kB, dh, dy, fD, bt, kG, ao, am, qb, aD, ha, dG, eq, ad, hX, fU, ceQ, hd, ib, aPn, av, byL, oz, oW, io, ji, lQ, d, lR, ff, eZ, dq, gE, aF, byM, aot, iR, iV, KM, bz, eK, iq, Nq, LB, D, xZ, bE, aS, cP, lC, tP, KS, X, jr, BA, pC); #c1(NFE2L2) c2(NP_006155) c3(nuclear factor erythroid 2-related factor 2 isoform 1) c4(162429, 162430) c5(162431, 162432, 162433, 162434, 162435, 162436, 162437, 162438, 162439, 162440) c6( ) c7(oO, bM, T, aC, fV, ea, e, al, if, bB, qj, aY, fw, sD, aR, bv, aE, dV, K, aDZ, eL, bc, N, Zm, aa, LC, aW, fc, hl, nY, Qq, aB, g, hJ, cm, fT, eM, nB, Kv, U, cj, NI, vy, tG, wx, Hb, m, bi, iu, FW, gU, dD, JY, aWG, n, BH, aj, asf, uE, PJ, qN, rP, Hx, ft, cc, bud, cU, lG, h, dL, hB, db, Nr, GZ, fJ, fQ, sm, aq, po, cM, adz, Ye, iO, W, dB, hk, Fi, Qu, hL, at, E, eo, cB, B, it, bL, eR, fG, hx, yk, aA, cR, fF, v, j, C, iI, V, q, J, er, eA, Wu, aT, cy, jj, an, ae, af, dl, Lu, s, oV, Y, r, lo, en, cl, t, iK, ck, sk, rR, nM, ac, c, kB, dh, dy, fD, bt, KG, ao, am, qb, aD, ha, dG, eq, ad, hX, fU, ceQ, hd, ib, aPn, av, byL, oz, oW, io, ji, lQ, d, lR, ff, eZ, dq, gE, aF, byM, aot, iR, iV, KM, bz, ek, iq, Nq, LB, D, xZ, bE, aS, cP, lC, tP, KS, X, jr, BA, pC); #c1(NFE2L3) c2(NP_004280) c3(nuclear factor erythroid 2-related factor 3) c4(144799, 144800) c5(144801, 144802, 144803, 144804, 144805, 144806, 144807, 144808, 144809, 144810, 144811, 144812, 144813, 144814, 144815) c6( ) c7(dP, k, jh, hf); #c1(NF1A) c2(XP_011539817) c3(nuclear factor 1 A-type isoform X4) c4(338039) c5( ) c6( ) c7(ul, ac, afy, bof, aA, cm, aF, hp, f, fq, af); #c1(NF1B) c2(NP_001177666) c3(nuclear factor 1 B-type isoform 1) c4(72879) c5(72880, 72881, 72882, 72883, 72884, 72885) c6( ) c7(T, cjC, fR, bKc, atC, che, aA, AO, afb, qK, aT, aHs, rz, af, s, aW, aF, bow, pm, cm, f, kP, W, ac, hj, in); #c1(NF1B) c2(NP_001177667) c3(nuclear factor 1 B-type isoform 2) c4(72886, 72887) c5(72888, 72889, 72890, 72891, 72892, 72893) c6( ) c7(T, cjC, fR, bKc, atC, chc, aA, AO, afb, qK, aT, aHs, rz, af, s, aW, aF, bow, pm, cm, f, kP, W, ac, hj, in); #c1(NF1B) c2(NP_001269716) c3(nuclear factor 1 B-type isoform 4) c4( ) c5(102697, 102698, 102699, 102700, 102701) c6( ) c7(T, cjC, fR, bKc, atC, chc, aA, AO, afb, qK, aT, aHs, rz, af, s, aW, aF, bow, pm, cm, f, kP, W, ac, hj, in); #c1(NF1B) c2(NP_005587) c3(nuclear factor 1 B-type isoform 3) c4(157699) c5(157700, 157701, 157702, 157703, 157704, 157705, 157706, 157707, 157708, 157709, 157710, 157711, 157712, 157713, 157714, 157715, 157716, 157717, 157718) c6( ) c7(T, cjC, fR, bKc, atC, chc, aA, AO, afb, qK, aT, aHs, rz, af, s, aW, aF, bow, pm, cm, f, kP, W, ac, hj, in); #c1(NF1B) c2(XP_005251524) c3(nuclear factor 1 B-type isoform X1) c4(270975) c5(270976, 270977, 270978, 270979, 270980, 270981) c6( ) c7(T, cjC, fR, bKc, atC, chc, aA, AO, afb, qK, aT, aHs, rz, af, s, aW, aF, bow, pm, cm, f, kP, W, ac, hj, in); #c1(NF1B) c2(XP_005251526) c3(nuclear factor 1 B-type isoform X4) c4(270982, 270983) c5(270984, 270985, 270986, 270987, 270988, 270989, 270990) c6( ) c7(T, cjC, fR, bKc, atC, chc, aA, AO, afb, qK, aT, aHs, rz, af, s, aW, aF, bow, pm, cm, f, kP, W, ac, hj, in); #c1(NF1B) c2(XP_005251527) c3(nuclear factor 1 B-type isoform X7) c4(270991) c5(270992, 270993, 270994, 270995, 270996, 270997) c6( ) c7(T, cjC, fR, bKc, atC, chc, aA, AO, afb, qK, aT, aHs, rz, af, s, aW, af, bow, pm, cm, f, kP, W, ac, hj, in); #c1(NF1B) c2(XP_006716837) c3(nuclear factor 1 B-type isoform X5) c4(290520) c5( ) c6( ) c7(T, cjC, fR, bKc, atC, chc, aA, AO, afb, qK, aT, aHs, rz af, s, aW, aF, bow, pm, cm, f, kP, W, ac, hj, in); #c1(NF1C) c2(NP_001231931) c3 (nuclear factor 1 C-type isoform 1) c4(87737) c5(87738, 87739, 87740, 87741, 87742, 87743, 87744) c6( ) c7(T, kl, cjC, ac, Ke, Ew, aA, cm, aF, f, af, ad); #c1(NF1C) c2(NP_001231934) c3(nuclear factor 1 C-type isoform 4) c4(87745, 87746) c5(87747, 87748, 87749, 87750, 87751, 87752, 87753, 87754, 87755, 87756, 87757, 87758) c6( ) c7(T, kl, cjC, ac, Ke, Ew, aA, cm, aF, f, af, ad); #c1(NF1C) c2(NP_005588) c3(nuclear factor 1 C-type isoform 5) c4(157719) c5(157720, 157721, 157722, 157723, 157724, 157725, 157726, 157727, 157728, 157729, 157730) c6( ) c7(T, kl, cjC, ac, Ke, Ew, aA, cm, aF, f, af, ad); #c1(NF1C) c2(NP_995315) c3(nuclear factor 1 C-type isoform 2) c4(263796) c5(263797, 263798, 263799, 263800, 263801, 263802, 263803, 263804, 263805, 263806) c6( ) c7(T, kl, cjC, ac, Ke, Ew, aA, cm, aF, f, af, ad); #c1(NF1C) c2(XP_005259621) c3(nuclear factor 1 C-type isoform X3) c4(276207) c5( ) c6( ) c7(T, kl, cjC, ac, Ke, Ew, aA, cm, aF, f, af, ad); #c1(NFIL3) c2(NP_001276929) c3(nuclear factor interleukin-3-regulated protein) c4(108636) c5(108637, 108638, 108639, 108640, 108641, 108642, 108643, 108644, 108645, 108646, 108647, 108648) c6( ) c7(T, zw, ac, c, xU, eZ, fe, ji, iV, Z, y); #c1(NFIX) c2(NP_001257972) c3(nuclear factor 1 X-type isoform 1) c4( ) c5(95879) c6( ) c7(ul, cjD, cjF, aPH, bof, aA, cm, aF, bK, f, af, cjE); #c1(NFIX) c2(NP_001257973) c3(nuclear factor 1 X-type isoform 3) c4(95880) c5( ) c6( ) c7(ul, cjD, cjF, aPH, bof, aA, cm, aF, bK, f, af, cjE); #c1(NFIX) c2(NP_002492) c3(nuclear factor 1 X-type isoform 2) c4(129288) c5(129289, 129290, 129291, 129292, 129293, 129294) c6( ) c7(ul, cjD, cjF, aPH, bof, aA, cm, aF, bK, f, af, cjE); #c1(NFIX) c2(XP_005259974) c3(nuclear factor 1 X-type isoform X1) c4(276333) c5(276334, 276335, 276336, 276337, 276338, 276339) c6( ) c7(ul, cjD, cjF, aPH, bof, aA, cm, aF, bK, f, af, cjE); #c1(NFIX) c2(XP_005259975) c3(nuclear factor 1 X-type isoform X3) c4(276340) c5(276341, 276342, 276343, 276344, 276345, 276346) c6( ) c7(ul, cjD, cjF, aPH, bof, aA, cm, aF, bK, f, af, cjE); #c1(NFIX) c2(XP_005259979) c3(nuclear factor 1 X-type isoform X8) c4( ) c5(276347, 276348, 276349, 276350, 276351) c6( ) c7(ul, cjD, cjF, aPH, bof, aA, cm, aF, bk, f, af, cjE); #c1(NFIX) c2(XP_006722823) c3(nuclear factor 1 X-type isoform X6) c4(294892) c5(294893, 294894, 294895, 294896, 294897, 294898) c6( ) c7(ul, cjD, cjF, aPH, bof, aA, cm, aF, bK, f, af, cjE); #c1(NFKB1) c2(NP_001158884) c3 (nuclear factor NF-kappa-B p105 subunit isoform 2) c4( ) c5(63748, 63749, 63750, 63751, 63752, 63753, 63754, 63755) c6( ) c7(oO, bM, T, os, aC, fR, pf, ea, bkF, e, abM, al, if, fd, bB, ci, aR, ip, x, bv, AW, wD, xE, dY, aE, anV, pX, dV, rB, K, hu, zy, hH, pk, eL, bc, cp, aa, DA, eG, ago, HI, aAg, aW, Jl, az, hl, aAu, Ls, fc, NR, g, as, eF, lk, uC, cm, bw, eM, Jr, xD, dc, nB, iA, iT, mx, vy, cj, uo, uJ, k, m, Uw, hh, fy, cjG, gD, atO, iu, dt, dD, aEj, sQ, aiK, aw, yG, ht, NT, n, aj, BF, fr, uE, qN, alq, cd, ai, eW, Vs, baA, ft, jz, pe, zw, kH, cU, Ne, h, hn, dL, ox, Jn, bl, eV, he, vx, wC, baj, Nr, bvO, mR, eZ, MD, Cw, A, gi, lb, ks, agb, aq, aga, kp, po, fe, dw, cM, Zb, Wt, afb, KT, bP, dB, w, aOL, hL, at, uz, ut, bm, fL, Pr, B, it, cn, cjJ, bL, ul, cjH, fM, Dw, eR, lH, jx, eX, rf, aA, cz, ti, fF, v, j, C, il, V, q, J, aZZ, eA, eJ, oT, mP, yw, bjA, Vr, apJ, LW, BJ, aT, qU, cy, bK, jj, an, rz, ae, af, eu, bA, jf, le, qw, cjl, s, iU, oV, Y, xR, Pg, r, lo, tu, en, cl, t, iK, pb, ck, nM, kP, c, ac, agc, ig, hc, MB, dh, iM, gX, dy, EY, JH, fD, UF, aps, bt, ao, Kd, sY, hl, id, aD, rQ, df, iW, jh, oZ, dG, eq, jE, agz, iJ, mC, ad, kL, hX, Jm, dP, av, hd, F, agd, py, iC, bjc, ds, NS, azq, io, hD, ji, lQ, Dn, cr, WZ, uQ, d, je, bja, Gv, ff, xs, wc, cjk, eH, dN, gl, tv, dq, hp, aeV, aF, iV, iR, KM, bz, DF, ek, Nq, LB, D, es, age, eU, zr, atM, yE, bE, Ny, dO, bEs, sG, aS, aGg, bC, ei, y, kS, X, hv); #c1(NFKB1) c2(NP_003989) c3(nuclear factor NF-kappa-B p105 subunit isoform 1) c4(142673, 142674) c5(142675, 142676, 142677, 142678, 142679, 142680) c6( ) c7(oO, bM, T, os, aC, fR, pf, ea, bkF, e, abM, al, if, fd, bB, ci, aR, ip, x, bv, AW, wD, xE, dY, aE, anV, pX, dV, rB, K, hu, zy, hH, pk, eL, bc, cp, aa, DA, eG, ago, HI, aAg, aW, Jl, az, hl, aAu, Ls, fc, NR, g, as, eF, lk, uC, cm, bw, eM, Jr, xD, dc, nB, iA, iT, mx, vy, cj, uo, uJ, k, m, Uw, hh, fy, cjG, gD, atO, iu, dt, dD, aEj, sQ, aiK, aw, yG, ht, NT, n, aj, BF, fr, uE, qN, alq, cd, ai, eW, Vs, baA, ft, jz, pe, zw, kH, cU, Ne, h, hn, dL, ox, Jn, bl, eV, he, vx, wC, baj, Nr, bvO, mR, eZ, MD, Cw, A, gi, lb, ks, agb, aq, aga, kp, po, fe, dw, cM, Zb, Wt, afb, kT, bP, dB, w, aOL, hl, at, uz, ut, bm, fL, Pr, B, it, cn, cjJ, bL, ul, cjH, fM, Dw, eR, lH, jx, eX, rf, aA, cz, ti, fF, v, j, C, il, V, q, J, aZZ, eA, eJ, oT, mP, yw, bjA, Vr, apJ, LW, BJ, aT, qU, cy, bK, jj, an, rz, ae, af, eu, bA, jf, le, qw, cjl, s, iU, oV, Y, xR, Pg, r, lo, tu, en, cl, t, iK, pb, ck, nM, kP, c, ac, agc, ig, hc, MB, dh, iM, gX, dy, EY, JH, fD, UF, aps, bt, ao, Kd, sY, hl, id, aD, rQ, df, iW, jh, oZ, dG, eq, jE, agz, iJ, mC, ad, kL, hX, Jm, dP, av, hd, F, agd, py, iC, bjc, ds, NS, azq, io, hD, ji, lQ, Dn, cr, WZ, uQ, d, je, bja, Gv, ff, xs, wc, cjk, eH, dN, gl, tv, dq, hp, aeV, aF, iV, iR, KM, bz, DF, ek, Nq, LB, D, es, age, eU, zr, atM, yE, bE, Ny, dO, bEs, sG, aS, aGg, bC, ei, y, kS, X, hv); #c1(NFKB1) c2(XP_011530308) c3(nuclear factor NF-kappa-B p105 subunit isoform X2) c4(326147, 326148) c5(326149, 326150, 326151, 326152, 326153, 326154, 326155, 326156) c6( ) c7(oO, bM, T, os, aC, fR, pf, ea, bkF, e, abM, al, if, fd, bB, ci, aR, ip, x, bv, AW, wD, xE, dY, aE, anV, pX, dV, rB, K, hu, zy, hH, pk, eL, bc, cp, aa, DA, eG, ago, HI, aAg, aW, Jl, az, hl, aAu, Ls, fc, NR, g, as, eF, lk, uC, cm, bw, eM, Jr, xD, dc, nB, iA, iT, mx, vy, cj, uo, uJ, k, m, Uw, hh, fy, cjG, gD, atO, iu, dt, dD, aEj, sQ, aiK, aw, yG, ht, NT, n, aj, BF, fr, uE, qN, alq, cd, ai, eW, Vs, baA, ft, jz, pe, zw, kH, cU, Ne, h, hn, dL, ox, Jn, bl, eV, he, vx, wC, baj, Nr, bvO, mR, eZ, MD, Cw, A, gi, lb, ks, agb, aq, aga, kp, po, fe, dw, cM, Zb, Wt, afb, kT, bP, dB, w, aOL, hl, at, uz, ut, bm, fL, Pr, B, it, cn, cjJ, bL, ul, cjH, fM, Dw, eR, lH, jx, eX, rf, aA, cz, ti, fF, v, j, C, il, V, q, J, aZZ, eA, eJ, oT, mP, yw, bjA, Vr, apJ, LW, BJ, aT, qU, cy, bK, jj, an, rz, ae, af, eu, bA, jf, le, qw, cjl, s, iU, oV, Y, xR, Pg, r, lo, tu, en, cl, t, iK, pb, ck, nM, kP, c, ac, agc, ig, hc, MB, dh, iM, gX, dy, EY, JH, fD, UF, aps, bt, ao, Kd, sY, hl, id, aD, rQ, df, iW, jh, oZ, dG, eq, jE, agz, iJ, mC, ad, kL, hX, Jm, dP, av, hd, F, agd, py, iC, bjc, ds, NS, azq, io, hD, ji, lQ, Dn, cr, WZ, uQ, d, je, bja, Gv, ff, xs, wc, cjk, eH, dN, gl, tv, dq, hp, aeV, aF, iV, iR, KM, bz, DF, ek, Nq, LB, D, es, age, eU, zr, atM, yE, bE, Ny, dO, bEs, sG, aS, aGg, bC, ei, y, kS, X, hv); #c1(NFKB1) c2(XP_011530310) c3(nuclear factor NF-kappa-B p105 subunit isoform X3) c4( ) c5(326157, 326158, 326159, 326160, 326161, 326162) c6( ) c7(oO, bM, T, os, aC, fR, pf, ea, bkF, e, abM, al, if, fd, bB, ci, aR, ip, x, bv, AW, wD, xE, dY, aE, anV, pX, dV, rB, K, hu, zy, hH, pk, eL, bc, cp, aa, DA, eG, ago, HI, aAg, aW, Jl, az, hl, aAu, Ls, fc, NR, g, as, eF, lk, uC, cm, bw, eM, Jr, xD, dc, nB, iA, iT, mx, vy, cj, uo, uJ, k, m, Uw, hh, fy, cjG, gD, atO, iu, dt, dD, aEj, sQ, aiK, aw, yG, ht, NT, n, aj, BF, fr, uE, qN, alq, cd, ai, eW, Vs, baA, ft, jz, pe, zw, kH, cU, Ne, h, hn, dL, ox, Jn, bl, eV, he, vx, wC, baj, Nr, bvO, mR, eZ, MD, Cw, A, gi, lb, ks, agb, aq, aga, kp, po, fe, dw, cM, Zb, Wt, afb, kT, bP, dB, w, aOL, hL, at, uz, ut, bm, fL, Pr, B, it, cn, cjJ, bL, ul, cjH, fM, Dw, eR, lH, jx, eX, rf, aA, cz, ti, fF, v, j, C, iI, V, q, J, aZZ, eA, eJ, oT, mP, yw, bjA, Vr, apJ, LW, BJ, aT, qU, cy, bK, jj, an, rz, ae, af, eu, bA, jf, le, qw, cjl, s, iU, oV, Y, xR, Pg, r, lo, tu, en, cl, t, iK, pb, ck, nM, kP, c, ac, agc, ig, hc, MB, dh, iM, gX, dy, EY, JH, fD, UF, aps, bt, ao, Kd, sY, hl, id, aD, rQ, df, iW, jh, oZ, dG, eq, jE, agz, iJ, mC, ad, kL, hX, Jm, dP, av, hd, F, agd, py, iC, bjc, ds, NS, azq, io, hD, ji, lQ, Dn, cr, WZ, uQ, d, je, bja, Gv, ff, xs, wc, cjK, eH, dN, gl, tv, dq, hp, aeV, aF, iV, iR, KM, bz, DF, ek, Nq, LB, D, es, age, eU, zr, atM, yE, bE, Ny, dO, bEs, sG, aS, aGg, bC, ei, y, kS, X, hv); #c1(NFKB2) c2(NP_002493) c3(nuclear factor NF-kappa-B p100 subunit isoform b) c4(129295) c5(129296, 129297, 129298, 129299, 129300, 129301, 129302, 129303, 129304, 129305) c6( ) c7(bM, T, dD, fM, dA, Dw, sQ, aC, ea, iW, al, jh, aA, bB, fd, eq, ci, qN, fr, alq, eA, ad, x, aE, dV, aT, kH, h, Ds, dz, Ze, lQ, hn, bq, ji, pk, ae, wC, je, MD, bNc, ff, aF, en, Ca, g, t, lk, iK, iV, Wt, ck, iM, bP, iT, bE, cjL, ahC, uo, cj, ut, k, aGg, aps, bt, zA, y); #c1(NFKB2) c2(XP_005269918) c3(nuclear factor NF-kappa-B p100 subunit isoform X1) c4(282996) c5(282997, 282998, 282999, 283000, 283001, 283002, 283003, 283004, 283005, 283006) c6( ) c7(bM, T, dD, fM, dA, Dw, sQ, aC, ea, iW, al, jh, aA, bB, fd, eq, ci, qN, fr, alq, eA, ad, x, aE, dV, aT, kH, h, Ds, dz, Ze, lQ, hn, bq, ji, pk, ae, wC, je, MD, bNc, ff, aF, en, Ca, g, t, lk, iK, iV, Wt, ck, iM, bP, iT, bE, cjL, ahC, uo, cj, ut, k, aGg, aps, bt, zA, y); #c1(NFKB1A) c2(NP_065390) c3(NF-kappa-B inhibitor alpha) c4(204683) c5(204684, 204685, 204686, 204687, 204688, 204689) c6( ) c7(oO, bM, T, aC, fR, if, al, fd, bB, dY, aE, Ds, K, hH, pk, aa, aW, g, eE, eF, eM, U, uo, Dt, k, m, hh, dD, sQ, aw, fN, oY, qN, pM, eW, Oe, cU, jy, h, eV, wb, aDp, A, mR, Cw, zb, fe, aq, Og, CQ, cjM, dB, Qu, at, aEG, ut, fL, it, B, Dw, eX, aA, fF, GC, j, C, q, eA, bjA, bjv, aT, cjN, dz, ae, eu, jf, qw, Y, r, lo, en, t, ac, fD, iW, jh, eq, ad, kL, hX, wD, av, hD, vB, ji, lQ, uQ, d, ff, eH, Of, cr, eZ, aF, iV, bz, bE, z, aS, pk, y, ra, X); #c1(NFKB1B) c2(NP_002494) c3(NF-kappa-B inhibitor beta isoform 1) c4(129306, 129307) c5(129308, 129309, 129310, 129311, 129312, 129313, 129314, 129315, 129316, 129317, 129318, 129319, (29320) c6( ) c7(dD, BN, h, sQ, at, iW, jx, uo, cjN, aEG, ut, hp, m, k, lQ, hy, t, eq, ne, y); #c1(NFKB1B) c2(XP_006723289) c3(NF-kappa-B inhibitor beta isoform X2) c4(295093) c5(295094, 295095, 295096, 295097, 295098, 295099) c6( ) c7(dD, BN, h, sQ, at, iW, jx, uo, cjN, aEG, ut, hp, m, k, lQ, hy, t, eq, ne, y); #c1(NFKB1E) c2(NP_004547) c3(NF-kappa-B inhibitor epsilon) c4(147492, 147493) c5(147494, 147495, 147496, 147497, 147498, 147499, 147500, 147501, 147502, 147503, 147504, 147505, 147506, 147507) c6( ) c7(dD, fR, sQ, aT, uo, cjN, ut, dL, eq, lQ, at, h, fd, y); #c1(NFKBIL1) c2(NP_001138433) c3(NF-kappa-B inhibitor-like protein 1 isoform 2) c4(56688, 56689) c5(56690, 56691, 56692, 56693, 56694, 56695, 56696, 56697, 56698, 56699, 56700, 56701, 56702, 56703, 56704, 56705, 56706, 56707, 56708, 56709, 56710, 56711, 56712, 56713, 56714, 56715, 56716, 56717) c6( ) c7(P, bM, iW, aw, fN, C, V, aue, cc, ds, lC, dV, hD, hH, aiJ, A, ue, eZ, pD, iV, uz, y); #c1(NFKBIL1) c2(NP_001138434) c3(NF-kappa-B inhibitor-like protein 1 isoform 3) c4( ) c5(56718, 56719, 56720, 56721, 56722, 56723, 56724, 56725, 56726, 56727, 56728, 56729, 56730, 56731, 56732, 56733, 56734, 56735, 56736, 56737, 56738, 56739, 56740, 56741, 56742, 56743, 56744, 56745, 56746, 56747, 56748, 56749, 56750, 56751, 56752, 56753, 56754, 56755, 56756, 56757, 56758, 56759, 56760, 56761, 56762, 56763, 56764, 56765, 56766, 56767) c6( ) c7(P, bM, iW, aw, fN, C, V, aue, cc, ds, lC, dV, hD, hH, aiJ, A, ue, eZ, pD, iV, uz, y); #c1(NFKBIL1) c2(NP_004998) c3 (NF-kappa-B inhibitor-like protein 1 isoform 1) c4(151856, 151857, 151858, 151859, 151860, 151861) c5(151862, 151863, 151864, 151865, 151866, 151867, 151868, 151869, 151870, 151871, 151872, 151873, 151874, 151875, 151876, 151877, 151878, 151879, 151880, 151881, 151882, 151883, 151884, 151885, 151886, 151887, 151888, 151889, 151890, 151891, 151892, 151893, 151894, 151895, 151896) c6( ) c7(P, bM, iW, aw, fN, C, V, aue, cc, ds, lC, dV, hD, hH, aiJ, A, ue, eZ, pD, iV, uz, y); #c1(NFKBIZ) c2(NP_001005474) c3 (NF-kappa-B inhibitor zeta isoform b) c4(21703, 21704, 21705) c5(21706, 21707, 21708, 21709, 21710, 21711, 21712, 21713, 21714, 21715, 21716, 21717, 21718, 21719, 21720, 21721, 21722, 21723, 21724, 21725, 21726) c6( ) c7(xS, Dy, dV, eE, cm, V, sj); #c1(NFKBIZ) c2(NP_113607) c3 (NF-kappa-B inhibitor zeta isoform a) c4(221698, 221699, 221700) c5(221701, 221702, 221703, 221704, 221705, 221706, 221707, 221708, 221709, 221710, 221711, 221712, 221713, 221714, 221715, 221716) c6( ) c7(xS, Dy, dV, eE, cm, V, sj); #c1(NFRKB) c2(NP_001137307) c3(nuclear factor related to kappa-B-binding protein isoform 1) c4(55430) c5( ) c6( ) c7(af, fd); #c1(NFRKB) c2(XP_011541151) c3(nuclear factor related to kappa-B-binding protein isoform X3) c4( ) c5(339695, 339696, 339697, 339698, 339699, 339700, 339701, 339702, 339703, 339704, 339705, 339706, 339707) c6( ) c7(af, fd); #c1(NFRKB) c2(XP_011541152) c3(nuclear factor related to kappa-B-binding protein isoform X4) c4( ) c5(339708) c6( ) c7(af, fd); #c1(NFRKB) c2(XP_011541154) c3(nuclear factor related to kappa-B-binding protein isoform X1) c4(339709) c5(339710, 339711, 339712, 339713, 339714, 339715, 339716) c6( ) c7(af, fd); #c1(NFS1) c2(NP_001185918) c3(cysteine desulfurase, mitochondrial isoform b precursor) c4(76757, 76758) c5(76759, 76760, 76761, 76762, 76763, 76764, 76765, 76766, 76767, 76768, 76769, 76770, 76771, 76772, 76773, 76774, 76775) c6( ) c7(cm); #c1(NFS1) c2(NP_066923) c3(cysteine desulfurase, mitochondrial isoform a precursor) c4(207485, 207486, 207487) c5(207488, 207489, 207490, 207491, 207492, 207493, 207494, 207495, 207496, 207497, 207498, 207499, 207500) c6( ) c7(cm); #c1(NFU1) c2(NP_001002755) c3(NFU1 iron-sulfur cluster scaffold homolog, mitochondrial isoform 2) c4(19562, 19563, 19564, 19565, 19566) c5(19567, 19568, 19569, 19570, 19571, 19572, 19573) c6( ) c7(hb, cjO, kC, aNG, yT); #c1(NFU1) c2(NP_001002756) c3(NFU1 iron-sulfur cluster scaffold homolog, mitochondrial isoform 3) c4( ) c5(19574, 19575, 19576, 19577, 19578, 19579, 19580, 19581, 19582, 19583, 19584, 19585, 19586, 19587, 19588, 19589, 19590, 19591) c6( ) c7(hb, cjO, kC, aNG, yT); #c1(NFU1) c2(NP_056515) c3(NFU1 iron-sulfur cluster scaffold homolog, mitochondrial isoform 1) c4( ) c5(187883, 187884, 187885, 187886, 187887, 187888, 187889, 187890, 187891) c6( ) c7(hb, cjO, kC, aNG, yT); #c1(NFYA) c2(NP_002496) c3(nuclear transcription factor Y subunit alpha isoform 1) c4(129321) c5( ) c6( ) c7(av, qh, m, K, qM, g, j, bz, B, qa, bc); #c1(NFYA) c2(NP_068351) c3(nuclear transcription factor Y subunit alpha isoform 2) c4(209304) c5(209305, 209306, 209307, 209308, 209309, 209310, 209311, 209312, 209313, 209314, 209315, 209316, 209317) c6( ) c7(av, qh, m, K, qM, g, j, bz, B, qa, bc); #c1(NFYB) c2(NP_006157) c3(nuclear transcription factor Y subunit beta) c4(162441) c5(162442, 162443, 162444, 162445, 162446, 162447, 162448, 162449, 162450, 162451, 162452) c6( ) c7(cm, bt, bz, qa); #c1(NFYC) c2(XP_011539819) c3(nuclear transcription factor Y subunit gamma isoform X5) c4(338040) c5( ) c6( ) c7(Qu, es, sZ, fr); #c1(NGB) c2(NP_067080) c3(neuroglobin) c4(208945) c5(208946, 208947, 208948, 208949, 208950, 208951) c6( ) c7(T, dD, d, ac, eM, dB, wO, ff, aAm, cy, eq, K, en, g, eK, cm, bz, af, eu, aeb); #c1(NGDN) c2(NP_001036100) c3(neuroguidin isoform 1) c4(33983) c5( ) c6( ) c7(beR, r, q, ae, X, ad); #c1(NGDN) c2(NP_056329) c3(neuroguidin isoform 2) c4(187115) c5(187116, 187117, 187118, 187119, 187120, 187121, 187122, 187123, 187124, 187125, 187126, 187127) c6( ) c7(beR, r, q, ae, X, ad); #c1(NGEF) c2(NP_001107562) c3(ephexin-1 isoform 2) c4(42843, 42844, 42845) c5(42846, 42847, 42848, 42849, 42850, 42851, 42852, 42853, 42854, 42855, 42856, 42857) c6( ) c7(v); #c1(NGEF) c2(XP_011509225) c3(ephexin-1 isoform X1) c4(299871, 299872) c5(299873, 299874, 299875, 299876, 299877, 299878, 299879, 299880, 299881, 299882, 299883, 299884) c6( ) c7(v); #c1(NGEF) c2(XP_011509227) c3(ephexin-1 isoform X2) c4( ) c5(299885, 299886, 299887, 299888, 299889, 299890) c6( ) c7(v); #c1(NGF) c2(XP_011539820) c3(beta-nerve growth factor isoform X2) c4(338041) c5(338042) c6( ) c7(bM, bB, aR, Ds, HR, H, hH, cjQ, aW, as, eE, cm, yX, wE, U, cZ, fN, aj, ct, ai, RD, biS, jz, cc, aqJ, dd, he, ES, sp, dw, cjP, hq, cA, dB, bOj, JM, jd, cB, B, L, Dw, eR, Lx, cz, j, bX, sl, V, q, J, uT, qK, aql, HC, aT, aaw, UN, bq, an, ae, af, jf, ak, kl, Y, abi, r, cu, en, nC, bN, ck, bdn, ac, lI, hc, cjR, JH, EY, gT, Pt, aFa, jh, fu, ad, wD, cjg, fU, uD, na, ff, cr, eZ, dr, Ew, xZ, bE, dJ, Ug); #c1(NGFR) c2(NP_002498) c3(tumor necrosis factor receptor superfamily member 16 precursor) c4(129322, 129323) c5(129324, 129325, 129326, 129327, 129328, 129329, 129330, 129331, 129332, 129333, 129334, 129335, 129336, 129337) c6( ) c7(bM, aBD, os, cZ, T, dA, ya, Dw, aC, Pt, lT, bO, aBx, aA, cz, oZ, bS, Fb, ct, ci, fu, bX, hF, aOM, cd, ad, eA, eW, x, aE, uT, h, tT, HC, aT, ab, cy, Ds, dL, hP, K, Ou, bq, dd, rz, j, ae, af, WZ, jf, kl, aW, Cu, Y, oV, apn, ff, aJW, aF, en, dw, eE, cm, dr, bN, ck, Hw, QG, nM, Ew, aBH, U, cj, dy, m, EY, it, B, ao, hS); #c1(NGFRAP1) c2(NP_001269603) c3 (protein BEX3 isoform c) c4(102170, 102171) c5(102172, 102173, 102174, 102175) c6( ) c7(hl); #c1(NGFRAP1) c2(NP_055195) c3(protein BEX3 isoform b) c4( ) c5(179700, 179701, 179702, 179703, 179704, 179705, 179706, 179707, 179708, 179709, 179710) c6( ) c7(hl); #c1(NGFRAP1) c2(NP_996800) c3(protein BEX3 isoform a) c4( ) c5(264567, 264568, 264569, 264570, 264571, 264572, 264573, 264574, 264575) c6( ) c7(hl); #c1(NGLY1) c2(NP_00138765) c3(peptide-N(4)-(N-acetyl-beta-glucosaminyl)asparagine amidase isoform 2) c4(57601, 57602, 57603) c5(57604, 57605, 57606, 57607, 57608, 57609, 57610, 57611, 57612, 57613, 57614, 57615) c6( ) c7(cjS); #c1(NGLY1) c2(NP_001138766) c3(peptide-N(4)-(N-acetyl-beta-glucosaminyl)asparagine amidase isoform 3) c4( ) c5(57616, 57617, 57618, 57619, 57620) c6( ) c7(cjS); #c1(NGLY1) c2(NP_001138767) c3(peptide-N(4)-(N-acetyl-beta-glucosaminyl)asparagine amidase isoform 4) c4(57621) c5(57622, 57623, 57624, 57625, 57626, 57627) c6( ) c7(cjS); #c1(NGLY1) c2(NP_060767) c3(peptide-N(4)-(N-acetyl-beta-glucosaminyl)asparagine amidase isoform 1) c4(197689, 197690) c5(197691, 197692, 197693, 197694, 197695, 197696, 197697) c6( ) c7(cjS); #c1(NGLY1) c2(XP_005265373) c3(peptide-N(4)-(N-acetyl-beta-glucosaminyl)asparagine amidase isoform X1) c4(279472) c5( ) c6( ) c7(cjS); #c1(NGLY1) c2(XP_011532246) c3(peptide-N(4)-(N-acetyl-beta-glucosaminyl)asparagine amidase isoform X2) c4( ) c5(328296, 328297) c6( ) c7(cjS); #c1(NGLY1) c2(XP_011532247) c3(peptide-N(4)-(N-acetyl-beta-glucosaminyl)asparagine amidase isoform X4) c4( ) c5(328298, 328299, 328300, 328301, 328302, 328303, 328304, 328305, 328306, 328307) c6( ) c7(cjS); #c1(NHEJ1) c2(NP_079058) c3(non-homologous end-joining factor 1) c4(216802, 216803, 216804) c5(216805, 216806, 216807, 216808, 216809, 216810, 216811, 216812, 216813, 216814, 216815, 216816, 216817, 216818, 216819, 216820, 216821, 216822) c6( ) c7(WL, bZr, K, bC, QK, V); #c1(NHLH1) c2(NP_005589) c3(helix-loop-helix protein 1) c4(157731) c5(157732, 157733, 157734, 157735, 157736, 157737, 157738) c6( ) c7(Na, T, Qy, Gc, atf, An, g, cm, asX); #c1(NHLRC1) c2(NP_940988) c3(E3 ubiquitin-protein ligase NHLRC1) c4(260267) c5(260268, 260269, 260270, 260271, 260272, 260273) c6( ) c7(yr, ha, cjT, U, jh, yT, yZ, yX, K, yA, yU, xg, xt); #c1(NHLRC3) c2(NP_001012772) c3(NHL repeat-containing protein 3 isoform a precursor) c4(25324) c5(25325, 25326, 25327, 25328, 25329, 25330, 25331, 25332) c6(25333, 25334) c7(Ea); #c1(NHLRC3) c2(NP_001017370) c3(NHL repeat-containing protein 3 isoform b) c4(26346, 26347) c5(26348, 26349, 26350, 26351, 26352, 26353, 26354, 26355, 26356, 26357, 26358, 26359, 26360, 26361, 26362, 26363) c6(26364, 26365, 26366, 26367) c7(Ea); #c1(NHP2) c2(NP_001030005) c3(H/ACA ribonucleoprotein complex subunit 2 isoform b) c4(30817) c5(30818, 30819, 30820, 30821, 30822) c6( ) c7(sb, av, cjW, cjU, cjV); #c1(NHP2) c2(NP_060308) c3(H/ACA ribonucleoprotein complex subunit 2 isoform a) c4(195301) c5(195302, 195303, 195304, 195305, 195306, 195307, 195308) c6( ) c7(sb, av, cjW, cjU, cjV); #c1(NHP2L1) c2(NP_004999) c3(NHP2-like protein 1) c4(151897, 151898) c5(151899, 151900, 151901, 151902, 151903, 151904, 151905, 151906, 151907, 151908, 151909, 151910, 151911, 151912, 151913, 151914, 151915, 151916, 151917, 151918) c6( ) c7(oZ, os); #c1(NHS) c2(NP_001129496) c3(Nance-Horan syndrome protein isoform 2) c4(51685, 51686) c5(51687, 51688, 51689, 51690, 51691, 51692, 51693, 51694, 51695, 51696, 51697, 51698) c6( ) c7(apV, eW, T, ec, aW, aZ, e, dV, aT, bD, ao, h, j, C, Ku, cjX, alV, q, Gc); #c1(NHS) c2(NP_001278796) c3(Nance-Horan syndrome protein isoform 3) c4( ) c5(110131) c6( ) c7(apV, eW, T, ec, aW, aZ, e, dV, aT, bD, ao, h, j, C, Ku, cjX, alV, q, Gc); #c1(NHS) c2(NP_938011) c3(Nance-Horan syndrome protein isoform 1) c4(259505) c5(259506, 259507, 259508, 259509, 259510, 259511) c6( ) c7(apV, eW, T, ec, aW, aZ, e, dV, aT, bD, ao, h, j, C, Ku, cjX, alV, q, Gc); #c1(NHSL1) c2(NP_001137532) c3(NHS-like protein 1 isoform 2) c4(56000) c5(56001, 56002, 56003, 56004, 56005, 56006) c6( ) c7(alV, af); #c1(NHSL1) c2(NP_065197) c3(NHS-like protein 1 isoform 1) c4(204554) c5(204555, 204556, 204557, 204558, 204559, 204560) c6( ) c7(alV, af); #c1(NICN1) c2(NP_115692) c3(nicolin-1) c4(223578, 223579) c5(223580, 223581, 223582, 223583, 223584, 223585, 223586, 223587, 223588, 223589, 223590, 223591, 223592, 223593, 223594, 223595) c6( ) c7(dL); #c1(NID1) c2(NP_002499) c3(nidogen-1 precursor) c4(129338) c5(129339, 129340, 129341, 129342, 129343, 129344, 129345, 129346) c6(129347, 129348) c7(T, bE, aW, Y, Nn, gX, aT, aA, bB, vy, ey, XT); #c1(NID1) c2(XP_011542497) c3(nidogen-1 isoform X1) c4(341040) c5(341041, 341042, 341043, 341044, 341045, 341046, 341047, 341048) c6(341049, 341050) c7(T, bE, aW, Y, Nn, gX, aT, aA, bB, vy, ey, XT); #c1(NID2) c2(NP_031387) c3 (nidogen-2 precursor) c4(172152, 172153, 172154) c5(172155, 172156, 172157, 172158, 172159, 172160, 172161, 172162, 172163, 172164, 172165, 172166, 172167, 172168, 172169, 172170, (72171, 172172) c6( ) c7(T, aW, hl, gX, aT, aA, dL, j, eL, B); #c1(NID2) c2(XP_005267464) c3(nidogen-2 isoform X3) c4(280983) c5(280984, 280985, 280986, 280987, 280988, 280989) c6( ) c7(T, aW, hl, gX, aT, aA, dL, j, eL, B); #c1(NIF3L1) c2(NP_001129511) c3 (putative GTP cyclohydrolase 1 type 2 NIF3L1 isoform 1) c4(51793, 51794, 51795, 51796, 51797) c5(51798, 51799, 51800, 51801, 51802, 51803, 51804, 51805, 51806, 51807, 51808, 51809, 51810, 51811, 51812) c6( ) c7(dD, yf, aji, J, ea, mx, cR, al, uJ, aj, eq, Wm, cm, aWT, ai, bAl, xw); #c1(NIF3L1) c2(NP_001135827) c3(putative GTP cyclohydrolase 1 type 2 NIF3L1 isoform 2) c4(53377) c5(53378, 53379, 53380, 53381, 53382) c6( ) c7(dD, yf, aji, J, ea, mx, cR, aU, uJ, aj, eq, Wm, cm, aWT, ai, bAl, xw); #c1(NIF3L1) c2(NP_001135828) c3(putative GTP cyclohydrolase 1 type 2 NIF3L1 isoform 3) c4(53383) c5(53384, 53385, 53386, 53387, 53388, 53389, 53390, 53391, 53392, 53393, 53394, 53395, 53396, 53397, 53398, 53399, 53400, 53401, 53402, 53403, 53404, 53405, 53406, 53407, 53408, 53409, 53410, 53411, 53412, 53413) c6( ) c7(dD, yf, aji, J, ea, mx, cR, aU, uJ, aj, eq, Wm, cm, aWT, ai, bAl, xw); #c1(NIM1K) c2(XP_006714513) c3(serine/threonine-protein kinase NIM1 isoform X1) c4(287881) c5(287882, 287883, 287884, 287885, 287886, 287887, 287888, 287889, 287890, 287891) c6( ) c7(Qq, hd, azh); #c1(NIN) c2(NP_057434) c3(ninein isoform 6) c4(190773) c5(190774, 190775, 190776, 190777, 190778, 190779) c6( ) c7(bVW, aE, et, dB, aW, aT, dz, cjY, Ea, QK, avx); #c1(NIN) c2(NP_065972) c3(ninein isoform 2) c4(206537, 206538) c5(206539, 206540, 206541, 206542, 206543, 206544, 206545, 206546, 206547, 206548, 206549, 206550, 206551, 206552, 206553, 206554, 206555, 206556) c6( ) c7(bVW, aE, et, dB, aW, aT, dz, cjY, Ea, QK, avx); #c1(NIN) c2(NP_891989) c3(ninein isoform 1) c4(257416) c5(257417, 257418, 257419, 257420, 257421, 257422) c6( ) c7(bVW, aE, et, dB, aW, aT, dz, cjY, Ea, QK, avx); #c1(NIN) c2(NP_891991) c3(ninein isoform 5) c4(257423) c5(257424, 257425, 257426, 257427, 257428, 257429) c6( ) c7(bVW, aE, et, dB, aW, aT, dz, cjY, Ea, QK, avx); #c1(NIN) c2(XP_005267792) c3(ninein isoform X8) c4(281246) c5( ) c6( ) c7(bVW, aE, et, dB, aW, aT, dz, cjY, Ea, QK, avx); #c1(NIN) c2(XP_006720223) c3(ninein isoform X5) c4(293322) c5(293323, 293324, 293325, 293326, 293327, 293328) c6( ) c7(bVW, aE, et, dB, aW, aT, dz, cjY, Ea, QK, avx); #c1(NIN) c2(XP_011535125) c3(ninein isoform X9) c4(331950) c5(331951, 331952, 331953, 331954, 331955, 331956) c6( ) c7(bVW, aE, et, dB, aW, aT, dz, cjY, Ea, QK, avx); #c1(NIN) c2(XP_011535126) c3(ninein isoform X10) c4(331957) c5(331958, 331959, 331960, 331961, 331962, 331963) c6( ) c7(bVW, aE, et, dB, aW, aT, dz, cjY, Ea, QK, avx); #c1(NINJ1) c2(NP_004139) c3(ninjurin-1) c4(143850) c5(143851, 143852, 143853, 143854, 143855, 143856) c6( ) c7(at, AW, dL, xU, fJ); #c1(NINJ2) c2(NP_001281274) c3(ninjurin-2 isoform 2) c4( ) c5(111676) c6( ) c7(uJ, eu, ac, M); #c1(NINJ2) c2(NP_001281275) c3(ninjurin-2 isoform 3) c4(111677) c5(111678, 111679, 111680, 111681, 111682) c6( ) c7(uJ, eu, ac, M); #c1(NINJ2) c2(NP_057617) c3(ninjurin- 2 isoform 1) c4(191804) c5(191805, 191806, 191807, 191808, 191809, 191810) c6( ) c7(uJ, eu, ac, M); #c1(NINL) c2(NP_079452) c3(ninein-like protein) c4(218165) c5(218166, 218167, 218168, 218169, 218170, 218171) c6( ) c7(T, aW, eX, jh, aT, eV, amY, bz); #c1(NIPA1) c2(NP_001135747) c3(magnesium transporter NIPA1 isoform 2) c4( ) c5(53012, 53013, 53014, 53015, 53016, 53017, 53018, 53019, 53020, 53021, 53022, 53023, 53024, 53025) c6( ) c7(cjZ, aiZ, Cq, no, c, aV, OI, ha, alR, U, ajb, agl, K, he, rc, Qn, bN); #c1(NIPA1) c2(NP_653200) c3(magnesium transporter NIPA1 isoform 1) c4(239032, 239033, 239034) c5(239035, 239036, 239037, 239038, 239039, 239040, 239041, 239042) c6( ) c7(cjZ, aiZ, Cq, no, c, aV, OI, ha, alR, U, ajb, agl, K, he, rc, On, bN); #c1(NIPA2) c2(NP_001008892) c3(magnesium transporter NIPA2 isoform a) c4(23450, 23451) c5(23452, 23453, 23454, 23455, 23456, 23457, 23458, 23459, 23460, 23461, 23462, 23463, 23464, 23465, 23466, 23467, 23468, 23469, 23470, 23471) c6( ) c7(HN, cM, rc, ad); #c1(NIPA2) c2(NP_001171817) c3(magnesium transporter NIPA2 isoform b) c4(71832) c5(71833, 71834, 71835, 71836, 71837, 71838, 71839, 71840, 71841, 71842) c6( ) c7(HN, cM, rc, ad); #c1(NIPAL3) c2(XP_011540110) c3(NIPA-like protein 3 isoform X3) c4( ) c5(338453, 338454, 338455, 338456, 338457, 338458, 338459, 338460, 338461, 338462, 338463) c6(338464, 338465) c7(ad); #c1(NIPAL4) c2(NP_001092757) c3(magnesium transporter NIPA4 isoform 1) c4(39614) c5(39615, 39616, 39617, 39618, 39619, 39620) c6( ) c7(fn, cd, cka); #c1(NIPAL4) c2(NP_001165763) c3(magnesium transporter NIPA4 isoform 2) c4(67885) c5(67886, 67887, 67888, 67889, 67890, 67891) c6( ) c7(fn, cd, cka); #c1(NIPBL) c2(NP_056199) c3(nipped-B-like protein isoform B) c4(186285) c5( ) c6( ) c7(bei, ff, Az, aNL, wR, en, zW, bD, ckb, X); #c1(NIPBL) c2(NP_597677) c3(nipped-B-like protein isoform A) c4(234976) c5(234977, 234978, 234979, 234980, 234981, 234982) c6( ) c7(bei, ff, Az, aNL, wR, en, zW, bD, ckb, X); #c1(NIPBL) c2(XP_005248337) c3(nipped-B-like protein isoform X3) c4( ) c5(268318, 268319, 268320, 268321, 268322, 268323) c6( ) c7(bei, ff, Az, aNL, wR, en, zW, bD, ckb, X); #c1(NIPSNAP1) c2(NP_003625) c3(protein NipSnap homolog 1 isoform 1) c4(139702) c5(139703, 139704, 139705, 139706, 139707, 139708) c6( ) c7(bQC); #c1(NIPSNAP3B) c2(NP_060846) c3 (protein NipSnap homolog 3B) c4(198110) c5(198111, 198112, 198113, 198114, 198115, 198116) c6( ) c7(cK, cJ); #c1(NISCH) c2(NP_001263222) c3(nischarin isoform 2) c4(97066) c5(97067, 97068, 97069, 97070, 97071, 97072) c6( ) c7(bL, dD, aW, ea, e, aT, cz, v, eq, cd); #c1(NISCH) c2(NP_009115) c3(nischarin isoform 1) c4(170798, 170799) c5(170800, 170801, 170802, 170803, 170804, 170805, 170806, 170807, 170808, 170809) c6( ) c7(bL, dD, aW, ea, e, al, cz, v, eq, cd); #c1(NISCH) c2(XP_006713018) c3(nischarin isoform X4) c4( ) c5(286962, 286963, 286964, 286965, 286966, 286967, 286968, 286969, 286970, 286971, 286972, 286973) c6( ) c7(bL, dD, aW, ea, e, aT, cz, v, eq, cd); #c1(NISCH) c2(XP_011531619) c3(nischarin isoform X1) c4(327766) c5( ) c6( ) c7(bL, dD, aW, ea, e, aT, cz, v, eq, cd); #c1(NIT1) c2(NP_001172021) c3(nitrilase homolog 1 isoform 2) c4(72257) c5(72258, 72259, 72260, 72261, 72262, 72263) c6( ) c7(jy, T, mj, K); #c1(NIT1) c2(NP_001172022) c3(nitrilase homolog 1 isoform 3) c4(72264, 72265) c5(72266, 72267, 72268, 72269, 72270, 72271) c6( ) c7(jy, T, mj, K); #c1(NIT1) c2(NP_001172023) c3(nitrilase homolog 1 isoform 4) c4( ) c5(72272, 72273, 72274, 72275, 72276) c6( ) c7(jy, T, mj, K); #c1(NIT1) c2(NP_005591) c3(nitrilase homolog 1 isoform 1) c4( ) c5(157739, 157740, 157741, 157742, 157743) c6( ) c7(jy, T, mj, K); #c1(NIT1) c2(XP_005245271) c3(nitrilase homolog 1 isoform X3) c4(266378) c5(266379) c6( ) c7(jy, T, mj, K); #c1(NIT1) c2(XP_005245272) c3(nitrilase homolog 1 isoform X2) c4( ) c5(266380) c6( ) c7(jy, T, mj, K); #c1(NIT2) c2(NP_064587) c3(omega-amidase NIT2) c4(203127, 203128) c5(203129, 203130, 203131, 203132, 203133, 203134, 203135, 203136, 203137, 203138, 203139, 203140) c6( ) c7(T); #c1(NKAIN2) c2(NP_001035304) c3(sodium/potassium-transporting ATPase subunit beta-l-interacting protein 2 isoform 1) c4(32758, 32759, 32760) c5(32761, 32762, 32763, 32764, 32765, 32766, 32767, 32768, 32769, 32770, 32771, 32772) c6( ) c7(ck, agJ, pH, ac, pN, Cw, cm, fq, cd, Hr); #c1(NKAIN2) c2(NP_001287666) c3(sodium/potassium-transporting ATPase subunit beta-l-interacting protein 2 isoform 3) c4(112492) c5(112493, 112494, 112495, 112496, 112497, 112498) c6( ) c7(ck, agJ, pH, ac, pN, Cw, cm, fq, cd, Hr); #c1(NKAIN2) c2(NP_699186) c3(sodium/potassium-transporting ATPase subunit beta-l-interacting protein 2 isoform 2) c4(245821) c5(245822, 245823, 245824, 245825, 245826, 245827) c6( ) c7(ck, agJ, pH, ac, pN, Cw, cm, fq, cd, Hr); #c1(NKAIN2) c2(XP_005266891) c3(sodium/potassium-transporting ATPase subunit beta-1-interacting protein 2 isoform X3) c4( ) c5(280370, 280371, 280372, 280373, 280374, 280375) c6( ) c7(ck, agJ, pH, ac, pN, Cw, cm, fq, cd, Hr); #c1(NKAIN3) c2(NP_001291462) c3(sodium/potassium-transporting ATPase subunit beta-l-interacting protein 3 precursor) c4(116115, 116116) c5( ) c6( ) c7(cm, ac); #c1(NKAIN3) c2(XP_011515813) c3(sodium/potassium-transporting ATPase subunit beta-1-interacting protein 3 isoform X1) c4( ) c5(308610, 308611, 308612, 308613, 308614, 308615, 308616, 308617, 308618, 308619, 308620, 308621, 308622, 308623, 308624, 308625) c6( ) c7(cm, ac); #c1(NKAP) c2(NP_078804) c3(NF-kappa-B-activating protein) c4(215552) c5(215553, 215554, 215555, 215556, 215557, 215558, 215559) c6( ) c7(y); #c1(NKD1) c2(NP_149110) c3(protein naked cuticle homolog 1) c4(227203) c5(227204, 227205, 227206, 227207, 227208, 227209) c6( ) c7(iO, T, s, eM, aX, r, lo, m, t, bz, q); #c1(NKD2) c2(NP_001258011) c3(protein naked cuticle homolog 2 isoform 2) c4(95894) c5(95895, 95896, 95897, 95898, 95899, 95900, 95901, 95902, 95903, 95904) c6( ) c7(hP, aF, d, aA); #c1(NKD2) c2(NP_149111) c3(protein naked cuticle homolog 2 isoform 1) c4(227210) c5(227211, 227212, 227213, 227214, 227215, 227216, 227217) c6( ) c7(hP, aF, d, aA); #c1(NKG7) c2(NP_005592) c3(protein NKG7) c4(157744) c5(157745, 157746, 157747, 157748, 157749, 157750) c6( ) c7(bt, eU, iU); #c1(NK1RAS1) c2(NP_065078) c3(NF-kappa-B inhibitor-interacting Ras-like protein 1) c4(203698) c5(203699, 203700, 203701, 203702, 203703, 203704, 203705, 203706, 203707, 203708, 203709, 203710, 203711, 203712, 203713, 203714, 203715, 203716, 203717, 203718, 203719, 203720) c6( ) c7(es, fr, y); #c1(NK1RAS2) c2(NP_001138400) c3(NF-kappa-B inhibitor-interacting Ras-like protein 2 isoform b) c4(56554) c5(56555, 56556, 56557, 56558, 56559, 56560) c6( ) c7(y, d); #c1(NK1RAS2) c2(NP_001138401) c3(NF-kappa-B inhibitor-interacting Ras-like protein 2 isoform c) c4(56561) c5(56562, 56563, 56564, 56565, 56566, 56567, 56568, 56569) c6( ) c7(y, d); #c1(NK1RAS2) c2(NP_060065) c3(NF-kappa-B inhibitor-interacting Ras-like protein 2 isoform a) c4(193983, 193984, 193985) c5(193986, 193987, 193988, 193989, 193990, 193991, 193992, 193993, 193994, 193995, 193996, 193997, 193998, 193999, 194000, 194001, 194002, 194003, 194004, 194005, 194006, 194007, 194008, 194009, 194010, 194011, 194012, 194013, 194014, 194015, 194016, 194017, 194018, 194019) c6( ) c7(y, d); #c1(NK1RAS2) c2(XP_011522982) c3 (NF-kappa-B inhibitor-interacting Ras-like protein 2 isoform X2) c4( ) c5(317449) c6( ) c7(y, d); #c1(NKRF) c2(NP_001166958) c3(NF-kappa-B-repressing factor isoform 1) c4(68733) c5(68734, 68735, 68736, 68737, 68738, 68739, 68740, 68741, 68742) c6( ) c7(bL, X, aE, c); #c1(NKRF) c2(XP_011529667) c3(NF-kappa-B-repressing factor isoform X1) c4(325305) c5(325306, 325307, 325308, 325309, 325310, 325311, 325312, 325313) c6( ) c7(bL, X, aE, c); #c1(NKTR) c2(NP_005376) c3(NK-tumor recognition protein) c4(155558, 155559) c5(155560, 155561, 155562, 155563, 155564, 155565, 155566) c6( ) c7(h); #c1(NKTR) c2(XP_006713234) c3(NK-tumor recognition protein isoform X2) c4( ) c5(287059, 287060, 287061, 287062, 287063, 287064) c6( ) c7(h); #c1(NKTR) c2(XP_011532052) c3(NK-tumor recognition protein isoform X4) c4( ) c5(328109, 328110) c6( ) c7(h); #c1(NKX1-1) c2(NP_001277008) c3(NK1 transcription factor-related protein 1) c4(108976) c5(108977, 108978, 108979, 108980, 108981, 108982, 108983) c6( ) c7(Ng, no, lu); #c1(NKX1-2) c2(NP_001139812) c3(NK1 transcription factor-related protein 2) c4(59997, 59998) c5(59999, 60000, 60001, 60002, 60003, 60004, 60005, 60006, 60007, 60008, 60009, 60010) c6( ) c7(aHP, aHQ); #c1(NKX2-1) c2(NP_001073136) c3(homeobox protein Nkx-2.1 isoform 1) c4(35592, 35593) c5(35594, 35595, 35596, 35597, 35598, 35599, 35600, 35601, 35602, 35603, 35604, 35605, 35606, 35607) c6( ) c7(Fv, bM, T, ahf, dA, apr, zX, v, ci, fw, eW, er, Vp, fE, ft, hd, av, Db, nv, iI, tT, bW, RN, ayQ, aT, aU, hD, bpf, h, Fx, ckf, biQ, aa, kl, s, aus, ckc, Wi, Y, aW, NE, AQ, afe, ff, kt, ahI, ckd, en, cl, t, xw, bz, g, dr, rR, yf, ac, eM, nB, nM, Qy, wk, cke, nG, hL, at, m, fD, zl, gT, iu); #c1(NKX2-1) c2(NP_003308) c3(homeobox protein Nkx-2.1 isoform 2) c4(137337) c5(137338, 137339, 137340, 137341, 137342, 137343, 137344, 137345, 137346, 137347, 137348, 137349, 137350) c6( ) c7(Fv, bM, T, ahf, dA, apr, zX, v, ci, fw, eW, er, Vp, fE, ft, hd, av, Db, nv, iI, tT, bW, RN, ayQ, aT, aU, hD, bpf, h, Fx, ckf, biQ, aa, kl, s, aus, ckc, Wi, Y, aW, NE, AQ, afe, ff, kt, ahI, ckd, en, cl, t, xw, bz, g, dr, rR, yf, ac, eM, nB, nM, Qy, wk, cke, nG, hL, at, m, fD, zl, gT, iu); #c1(NKX2-2) c2(NP_002500) c3(homeobox protein Nkx-2.2) c4(129349) c5(129350, 129351, (29352, 129353, 129354, 129355) c6( ) c7(bL, T, ft, d, mh, Y, pX, as, mF, cm, Fc, eA, dx); #c1(NKX2-3) c2(NP_660328) c3(homeobox protein Nkx-2.3) c4(240941) c5(240942, 240943, 240944, 240945, 240946, 240947, 240948) c6( ) c7(xN, T, nG, r, aMA, fe, iV, ra, q); #c1(NKX2-5) c2(NP_001159647) c3(homeobox protein Nkx-2.5 isoform 2) c4( ) c5(64544, 64545, 64546, 64547, 64548) c6( ) c7(ckh, ahf, ckk, ckl, mr, atk, wL, ckm, fG, acl, aln, ckj, j, MC, bzq, ckn, cd, ad, md, Mg, cc, RN, acA, aT, hD, atw, adm, rz, chN, ae, cko, aW, eZ, Vt, ckg, si, aac, atb, dR, amF, acw, gY, gZ, cki, k, Km, aS, bD, y, KY, vQ); #c1(NKX2-5) c2(NP_001159648) c3(homeobox protein Nkx-2.5 isoform 3) c4(64549) c5(64550, 64551, 64552, 64553, 64554, 64555) c6( ) c7(ckh, ahf, ckk, ckl, mr, atk, wL, ckm, fG, acl, aln, ckj, j, MC, bzq, ckn, cd, ad, md, Mg, cc, RN, acA, aT, hD, atw, adm, rz, chN, ae, cko, aW, eZ, Vt, ckg, si, aac, atb, dR, amF, acw, gY, gZ, cki, k, Km, aS, bD, y, KY, vQ); #c1(NKX2-5) c2(NP_004378) c3(homeobox protein Nkx-2.5 isoform 1) c4(145930, 145931) c5(145932, 145933, 145934, 145935, 145936, 145937, 145938, 145939, 145940, 145941) c6( ) c7(ckh, ahf, ckk, ckl, mr, atk, wL, ckm, fG, acl, aln, ckj, j, MC, bzq, ckn, cd, ad, md, Mg, cc, RN, acA, aT, hD, atw, adm, rz, chN, ae, cko, aW, eZ, Vt, ckg, si, aac, atb, dR, amF, acw, gY, gZ, cki, k, Km, aS, bD, y, KY, vQ); #c1(NKX2-6) c2(NP_001129743) c3(homeobox protein Nkx-2.6) c4(52307, 52308) c5(52309, 52310, 52311, 52312, 52313, 52314, 52315, 52316, 52317, 52318, 52319, 52320) c6( ) c7(aLi, fq, C); #c1(NKX2-8) c2(NP_055175) c3(homeobox protein Nkx-2.8) c4(179577) c5(179578, 179579, 179580, 179581, 179582, 179583, 179584) c6( ) c7(T, eM, lp, eL, ht, it, aur, dL, hu, bz); #c1(NKX3-1) c2(NP_001243268) c3(homeobox protein Nkx-3.1 isoform 2) c4(90336) c5(90337, 90338, 90339, 90340, 90341, 90342, 90343, 90344, 90345, 90346, 90347, 90348) c6( ) c7(gh, G, T, wu, Fb, ci, ad, x, Bl, aT, hn, wm, aDZ, ae, li, Ub, vT, aW, Lf, ff, en, bu, g, ck, eD, Lb, k, in); #c1(NKX3-1) c2(NP_006158) c3(homeobox protein Nkx-3.1 isoform 1) c4(162453, 162454) c5(162455, 162456, 162457, 162458, 162459, 162460, 162461) c6( ) c7(gh, G, T, wu, Fb, ci, ad, x, Bl, aT, hn, wm, aDZ, ae, li, Ub, vT, aW, Lf, ff, en, bU, g, ck, eD, Lb, k, in); #c1(NKX6-1) c2(XP_006714293) c3(homeobox protein Nkx-6.1 isoform X1) c4(287675) c5(287676, 287677, 287678, 287679, 287680, 287681, 287682) c6( ) c7(bL, beR, bq); #c1(NKX6-2) c2(NP_796374) c3(homeobox protein Nkx-6.2) c4(252476, 252477) c5(252478, 252479, 252480, 252481, 252482, 252483, 252484, 252485, 252486, 252487, 252488, 252489, 252490) c6( ) c7(bL, dB, dG, dL, Fc, eL); #c1(NLE1) c2(NP_001014445) c3(notchless protein homolog 1 isoform b) c4(25927) c5(25928, 25929, 25930, 25931, 25932, 25933, 25934, 25935) c6( ) c7(pJ, T); #c1(NLE1) c2(NP_060566) c3(notchless protein homolog 1 isoform a) c4(196607, 196608) c5(196609, 196610, 196611, 196612, 196613, 196614, 196615, 196616, 196617, 196618, 196619, 196620, 196621, 196622) c6( ) c7(pJ, T); #c1(NLE1) c2(XP_011523215) c3(notchless protein homolog 1 isoform X1) c4(317738) c5(317739, 317740, 317741, 317742, 317743, 317744) c6( ) c7(pJ, T); #c1(NLGN1) c2(NP_055747) c3(neuroligin-1) c4(183251) c5(183252, 183253, 183254, 183255, 183256, 183257, 183258, 183259, 183260, 183261, 183262, 183263, 183264, 183265) c6( ) c7(cF, c, rz, aw, bK, Z, he, bN); #c1(NLGN1) c2(XP_005247290) c3(neuroligin-1 isoform X3) c4(267751) c5( ) c6( ) c7(cF, c, rz, aw, bK, Z, he, bN); #c1(NLGN1) c2(XP_005247293) c3 (neuroligin-1 isoform X4) c4( ) c5(267752) c6( ) c7(cF, c, rz, aw, bk, Z, he, bN); #c1(NLGN1) c2(XP_011510854) c3(neuroligin-1 isoform X2) c4(301305) c5(301306, 301307, 301308, 301309, 301310, 301311, 301312, 301313, 301314, 301315, 301316, 301317, 301318, 301319, 301320, 301321) c6( ) c7(cF, c, rz, aw, bk, Z, he, bN); #c1(NLGN2) c2(NP_065846) c3(neuroligin-2 precursor) c4(205826) c5(205827, 205828, 205829, 205830, 205831, 205832) c6( ) c7(bN); #c1(NLGN3) c2(NP_001160132) c3(neuroligin-3 isoform 3 precursor) c4(65091, 65092) c5(65093, 65094, 65095, 65096, 65097, 65098, 65099, 65100, 65101, 65102, 65103, 65104) c6( ) c7(aQy, ckp, hb, ckq, ckr, nA, rz, bN); #c1(NLGN3) c2(NP_061850) c3(neuroligin-3 isoform 2 precursor) c4(200778) c5(200779, 200780, 200781, 200782, 200783, 200784) c6( ) c7(aQy, ckp, hb, ckq, ckr, nA, rz, bN); #c1(NLGN3) c2(NP_851820) c3(neuroligin-3 isoform 1 precursor) c4(254601) c5(254602, 254603, 254604, 254605, 254606, 254607) c6( ) c7(aQy, ckp, hb, ckq, ckr, nA, rz, bN); #c1(NLGN3) c2(XP_006724726) c3(neuroligin-3 isoform X2) c4( ) c5(296277) c6( ) c7(aQy, ckp, hb, ckq, ckr, nA, rz, bN); #c1(NLGN3) c2(XP_011529277) c3(neuroligin-3 isoform X3) c4(324716) c5(324717, 324718, 324719, 324720, 324721) c6( ) c7(aQy, ckp, hb, ckq, ckr, nA, rz, bN); #c1(NLGN4X) c2(NP_001269075) c3(neuroligin-4, X-linked) c4(100370) c5(100371, 100372, 100373, 100374, 100375, 100376, 100377, 100378, 100379, 100380, 100381, 100382, 100383, 100384, 100385, 100386, 100387, 100388) c6( ) c7(bN, VX, rz, hb, ckt, yL, hQ, nf, btu, nA, he, cks, ckr); #c1(NLGN4X) c2(XP_011543850) c3(neuroligin-4, X-linked isoform X1) c4(342987) c5(342988, 342989, 342990, 342991, 342992, 342993) c6( ) c7(bN, VX, rz, hb, ckt, yL, hQ, nf, btu, nA, he, cks, ckr); #c1(NLGN4Y) c2(NP_001193779) c3(neuroligin-4, Y-linked isoform 3) c4(82600) c5(82601, 82602, 82603, 82604, 82605) c6( ) c7(bN, nA); #c1(NLGN4Y) c2(NP_055708) c3(neuroligin-4, Y-linked isoform 1 precursor) c4(182976) c5(182977, 182978, 182979, 182980, 182981, 182982, 182983, 182984, 182985, 182986) c6( ) c7(bN, nA); #c1(NLGN4Y) c2(XP_011529732) c3(neuroligin-4, Y-linked isoform X2) c4(325355, 325356) c5(325357, 325358, 325359, 325360, 325361, 325362, 325363, 325364, 325365, 325366, 325367, 325368, 325369, 325370) c6( ) c7(bN, nA); #c1(NLGN4Y) c2(XP_011529733) c3 (neuroligin-4, Y-linked isoform X1) c4(325371, 325372) c5(325373, 325374, 325375, 325376, 325377, 325378, 325379, 325380, 325381, 325382, 325383, 325384) c6( ) c7(bN, nA); #c1(NLK) c2(NP_057315) c3(serine/threonine-protein kinase NLK) c4(190067) c5(190068, 190069, 190070, 190071, 190072, 190073) c6( ) c7(T, B, eM, bE, aW, r, aT, bB, aq, m, iR, t, ae, X, ad); #c1(NLN) c2(NP_065777) c3(neurolysin, mitochondrial) c4(205349, 205350, 205351) c5(205352, 205353, 205354, 205355, 205356, 205357, 205358, 205359, 205360, 205361, 205362, 205363, 205364, 205365, 205366, 205367, 205368, 205369) c6( ) c7(T, os, af, aW, cr, aT, oZ, j, cm, ae, cd, ad, hZ); #c1(NLRC3) c2(NP_849172) c3(protein NLRC3) c4(254399, 254400, 254401, 254402, 254403) c5(254404, 254405, 254406, 254407, 254408, 254409, 254410, 254411, 254412, 254413, 254414, 254415, 254416, 254417, 254418, 254419, 254420, 254421, 254422, 254423, 254424, 254425, 254426, 254427, 254428, 254429, 254430, 254431, 254432, 254433) c6( ) c7(eW); #c1(NLRC4) c2(NP_001186068) c3(NLR family CARD domain-containing protein 4 isoform a) c4(77005) c5(77006, 77007, 77008, 77009, 77010, 77011, 77012, 77013, 77014, 77015, 77016, 77017, 77018, 77019) c6( ) c7(kV, oO, ds, apJ, aLJ, eZ, bsk, nN, ZN, NX); #c1(NLRC4) c2(NP_001289433) c3(NLR family CARD domain-containing protein 4 isoform b) c4(114205) c5(114206, 114207, 114208, 114209, 114210, 114211) c6( ) c7(kV, oO, ds, apJ, aLJ, eZ, bsk, nN, ZN, NX); #c1(NLRC5) c2(XP_005256251) c3(protein NLRC5 isoform X1) c4(274014, 274015) c5(274016, 274017, 274018, 274019, 274020, 274021, 274022, 274023, 274024, 274025, 274026, 274027, 274028, 274029, 274030, 274031, 274032) c6( ) c7(oO, bL, j); #c1(NLRC5) c2(XP_005256252) c3(protein NLRC5 isoform X2) c4( ) c5(274033, 274034, 274035, 274036, 274037) c6( ) c7(oO, bL, j); #c1(NLRC5) c2(XP_005256254) c3(protein NLRC5 isoform X4) c4(274038) c5(274039, 274040, 274041, 274042, 274043, 274044) c6( ) c7(oO, bL, j); #c1(NLRC5) c2(XP_005256258) c3(protein NLRC5 isoform X8) c4(274045, 274046) c5(274047, 274048, 274049, 274050, 274051, 274052) c6( ) c7(oO, bL, j); #c1(NLRC5) c2(XP_006721361) c3(protein NLRC5 isoform X5) c4(293952) c5( ) c6( ) c7(oO, bL, j); #c1(NLRC5) c2(XP_011521677) c3(protein NLRC5 isoform X10) c4( ) c5(316436, 316437) c6( ) c7(oO, bL, j); #c1(NLRP1) c2(NP_001028225) c3(NACHT, LRR and PYD domains-containing protein 1 isoform 5) c4(30378) c5(30379, 30380, 30381, 30382, 30383, 30384, 30385, 30386, 30387, 30388) c6( ) c7(T, mr, fG, aw, aA, cL, qj, fr, C, uE, hF, ai, o, ad, AW, apJ, h, K, hK, NM, ae, af, yF, A, Y, XV, iw, aFt, eZ, fe, af, hp, as, eE, cm, pb, es, cT, L, yl, ac, rR, iA, iT, cku, ajd, cj, U, y, Tr, iu, abh, pC); #c1(NLRP1) c2(NP_055737) c3(NACHT, LRR and PYD domains-containing protein 1 isoform 2) c4(183188, 183189) c5(183190, 183191, 183192, 183193, 183194, 183195) c6( ) c7(T, mr, fG, aw, aA, cL, qj, fr, C, uE, hF, ai, o, ad, AW, apJ, h, K, hK, NM, ae, af, yF, A, Y, XV, iw, aFt, eL, fe, af, hp, as, eE, cm, pb, es, cT, L, yl, ac, rR, iA, iT, cku, ajd, cj, U, y, Tr, iu, abh, pC); #c1(NLRP1) c2(NP_127497) c3(NACHT, LRR and PYD domains-containing protein 1 isoform 1) c4(226538) c5(226539, 226540, 226541, 226542, 226543, 226544, 226545, 226546, 226547, 226548, 226549, 226550, 226551, 226552, 226553, 226554) c6( ) c7(T, mr, fG, aw, aA, cl, qj, fr, C, uE, hF, ai, o, ad, AW, apJ, h, K, hK, NM, ae, af, yF, A, Y, XV, iw, aFt, eZ, fe, aF, hp, as, eE, cm, pb, es, cT, L, yl, ac, rR, iA, iT, cku, ajd, cj, U, y, Tr, iu, abh, pC); #c1(NLRP1) c2(NP_127499) c3(NACHT, LRR and PYD domains-containing protein 1 isoform 3) c4(226555) c5(226556, 226557, 226558, 226559, 226560, 226561) c6( ) c7(T, mr, fG, aw, aA, cl, qj, fr, C, uE, hF, ai, o, ad, AW, apJ, h, K, hK, NM, ae, af, yF, A, Y, XV, iw, aFt, eL, fe, aF, hp, as, eE, cm, pb, es, cT, L, yl, ac, rR, iA, iT, cku, ajd, cj, U, y, Tr, iu, abh, pC); #c1(NLRP1) c2(NP_127500) c3(NACHT, LRR and PYD domains-containing protein 1 isoform 4) c4(226562) c5(226563, 226564, 226565, 226566, 226567, 226568) c6( ) c7(T, mr, fG, aw, aA, c, qj, fr, C, uE, hF, ai, o, ad, AW, apJ, h, K, hK, NM, ae, af, yF, A, Y, XV, iw, aFt, eZ, fe, aF, hp, as, eE, cm, pb, es, cT, L, yl, ac, rR, iA, iT, cku, ajd, cj, U, y, Tr, iu, abh, pC); #c1(NLRP10) c2(XP_011518345) c3 (NACHT, LRR and PYD domains-containing protein 10 isoform X1) c4(312349, 312350) c5(312351, 312352, 312353, 312354, 312355, 312356, 312357, 312358, 312359, 312360, 312361, 312362) c6( ) c7(aj, J); #c1(NLRP11) c2(NP_659444) c3(NACHT, LRR and PYD domains-containing protein 11 isoform 1) c4(239925, 239926) c5(239927, 239928, 239929, 239930, 239931, 239932, 239933, 239934, 239935, 239936, 239937, 239938) c6( ) c7(V, gv, cj); #c1(NLRP12) c2(NP_001264055) c3(NACHT, LRR and PYD domains-containing protein 12 isoform 3) c4(97448, 97449) c5(97450, 97451, 97452, 97453, 97454, 97455, 97456, 97457, 97458, 97459, 97460, 97461) c6( ) c7(EW, bE, NY, apJ, dV, bB, fr, R, eE, ckv, NM, Wt); #c1(NLRP12) c2(NP_001264058) c3(NACHT, LRR and PYD domains-containing protein 12 isoform 4) c4(97462) c5(97463, 97464, 97465, 97466, 97467, 97468, 97469, 97470, 97471, 97472, 97473, 97474) c6( ) c7(EW, bE, NY, apJ, dV, bB, fr, R, eE, ckv, NM, Wt); #c1(NLRP12) c2(NP_653288) c3(NACHT, LRR and PYD domains-containing protein 12 isoform 2) c4(239504, 239505) c5(239506, 239507, 239508, 239509, 239510, 239511, 239512, 239513, 239514, 239515, 239516, 239517) c6( ) c7(EW, bE, NY, apJ, dV, bB, fr, R, eE, ckv, NM, Wt); #c1(NLRP12) c2(XP_011525782) c3(NACHT, LRR and PYD domains-containing protein 12 isoform X3) c4(320175) c5( ) c6( ) c7(EW, bE, NY, apJ, dV, bB, fr, R, eE, ckv, NM, Wt); #c1(NLRP12) c2(XP_011525784) c3(NACHT, LRR and PYD domains-containing protein 12 isoform X5) c4(320176, 320177, 320178) c5(320179, 320180, 320181, 320182, 320183, 320184, 320185, 320186, 320187, 320188, 320189, 320190, 320191, 320192, 320193, 320194, 320195, 320196) c6( ) c7(EW, bE, NY, apJ, dV, bB, fr, R, eE, ckv, NM, Wt); #c1(NLRP13) c2(NP_789780) c3(NACHT, LRR and PYD domains-containing protein (3) c4(252266) c5(252267, 252268, 252269, 252270, 252271, 252272) c6( ) c7(cj); #c1(NLRP14) c2(NP_789792) c3(NACHT, LRR and PYD domains-containing protein 14) c4(252312) c5(252313, 252314, 252315, 252316, 252317, 252318) c6( ) c7(vH, ND, cj); #c1(NLRP2) c2(NP_001167552) c3(NACHT, LRR and PYD domains-containing protein 2 isoform 1) c4(69071, 69072, 69073) c5(69074, 69075, 69076, 69077, 69078, 69079, 69080, 69081, 69082, 69083, 69084, 69085, 69086, 69087, 69088, 69089, 69090, 69091, 69092, 69093, 69094, 69095, 69096, 69097, 69098, 69099, 69100, 69101, 69102, 69103, 69104, 69105, 69106, 69107, 69108, 69109, 69110, 69111, 69112, 69113, 69114, 69115, 69116, 69117, 69118, 69119, 69120, 69121, 69122, 69123, 69124, 69125, 69126, 69127, 69128, 69129, 69130, 69131, 69132, 69133, 69134, 69135, 69136, 69137, 69138, 69139, 69140, 69141) c6( ) c7(T, aC, WL, iW, eX, ml, jh, aBx, n, oZ, Gn, fr, nA, bX, hF, q, ad, eL, x, aE, ld, aT, qv, qG, eV, ae, jf, qw, d, CN, Cw, aW, Y, CM, bg, eZ, r, aJW, lo, eS, g, QK, cM, bz, iK, dr, iP, adq, ck, ajl, ba, Dy, eM, cj, cx, dW, m, k, bC, bt, kS); #c1(NLRP2) c2(NP_001167553) c3(NACHT, LRR and PYD domains-containing protein 2 isoform 2) c4(69142) c5(69143, 69144, 69145, 69146, 69147, 69148, 69149, 69150, 69151, 69152, 69153, 69154, 69155, 69156, 69157, 69158, 69159, 69160, 69161, 69162, 69163, 69164, 69165, 69166, 69167, 69168, 69169, 69170, 69171, 69172, 69173) c6( ) c7(T, aC, WL, iW, eX, ml, jh, aBx, n, oZ, Gn, fr, nA, bX, hF, q, ad, eJ, x, aE, ld, aT, qv, qG, eV, ae, jf, qw, d, CN, Cw, aW, Y, CM, bg, eZ, r, aJW, lo, eS, g, QK, cM, bz, iK, dr, iP, adq, ck, ajl, ba, Dy, eM, cj, cX, dW, m, k, bC, bt, KS); #c1(NLRP2) c2(NP_00167554) c3(NACHT, LRR and PYD domains-containing protein 2 isoform 3) c4(69174) c5(69175, 69176, 69177, 69178, 69179, 69180) c6( ) c7(T, aC, WL, iW, eX, ml, jh, aBx, n, oZ, Gn, fr, nA, bX, hF, q, ad, eJ, x, aE, ld, aT, qv, qG, eV, ae, jf, qw, d, CN, Cw, aW, Y, CM, bg, eZ, r, aJW, lo, eS, g, QK, cM, bz, iK, dr, iP, adq, ck, ajl, ba, Dy, eM, cj, cX, dW, m, k, bC, bt, KS); #c1(NLRP3) c2(NP_001120933) c3(NACHT, LRR and PYD domains-containing protein 3 isoform c) c4(44963) c5(44964, 44965, 44966, 44967, 44968, 44969, 44970, 44971, 44972, 44973) c6( ) c7(bol, oO, T, fR, ea, bkF, cL, Ja, xE, fS, dV, iv, K, hH, pk, aa, aW, asD, ih, aoD, eE, cm, axp, Uk, ba, lt, qr, aiM, R, hh, fy, abh, nD, dD, aw, n, amk, NJ, aj, nA, aPb, ai, eW, aDF, ke, adN, h, ey, NX, A, mR, adF, fe, aq, mo, ajz, nN, cM, Zb, bQL, hq, iO, W, bm, hs, fL, ckw, bdR, B, adG, bL, Dw, aAf, fG, yk, jx, ew, cz, azF, v, j, C, V, q, J, o, nX, apJ, aT, qG, aAD, Fg, ae, bf, s, Y, RF, bg, aLJ, r, lo, aDm, Rp, ajl, rR, Uq, wR, dy, yH, fD, aPJ, bQH, jh, eq, mC, ad, afb, LH, wD, fU, F, ib, NY, ds, adD, ji, XI, nh, cr, eZ, dq, hp, iV, iR, YZ, qa, LB, bF, Ny, gZ, ei, Lz, i, y, MN); #c1(NLRP3) c2(XP_011542353) c3(NACHT, LRR and PYD domains-containing protein 3 isoform X3) c4(340914) c5(340915, 340916, 340917, 340918, 340919, 340920, 340921, 340922, 340923, 340924, 340925) c6( ) c7(bol, oO, T, fR, ea, bkF, cL, Ja, xE, fS, dV, iv, K, hH, pk, aa, aW, asD, ih, aoD, eE, cm, axp, Uk, ba, lt, qr, aiM, R, hh, fy, abh, nD, dD, aw, n, amk, NJ, aj, nA, aPb, ai, eW, aDF, ke, adN, h, ey, NX, A, mR, adF, fe, aq, mo, ajz, nN, cM, Zb, bQL, hq, iO, W, bm, hs, fl, ckw, bdR, B, adG, bL, Dw, aAf, fG, yk, jx, ew, cz, azF, v, j, C, V, q, J, o, nX, apJ, aT, qG, aAD, Fg, ae, bf, s, Y, RF, bg, aLJ, r, lo, aDm, Rp, ajl, rR, Uq, wR, dy, yH, fD, aPJ, bQH, jh, eq, mC, ad, afb, LH, wD, fU, F, ib, NY, ds, adD, ji, Xl, nh, cr, eZ, dq, hp, iV, iR, YZ, qa, LB, bF, Ny, gZ, ei, Lz, i, y, MN); #c1(NLRP3) c2(XP_011542354) c3(NACHT, LRR and PYD domains-containing protein 3 isoform X2) c4(340926) c5(340927, 340928, 340929, 340930, 340931, 340932) c6( ) c7(bol, oO, T, fR, ea, bkF, cL, Ja, xE, fS, dV, iv, K, hH, pk, aa, aW, asD, ih, aoD, eE, cm, axp, Uk, ba, lt, qr, aiM, R, hh, fy, abh, nD, dD, aw, n, amk, NJ, aj, nA, aPb, ai, eW, aDF, ke, adN, h, ey, NX, A, mR, adF, fe, aq, mo, ajz, nN, cM, Zb, bQL, hq, iO, W, bm, hs, fl, ckw, bdR, B, adG, bL, Dw, aAf, fG, yk, jx, ew, cz, azF, v, j, C, V, q, J, o, nX, apJ, aT, qG, aAD, Fg, ae, bf, s, Y, RF, bg, aLJ, r, lo, aDm, Rp, ajl, rR, Uq, wR, dy, yH, fD, aPJ, bQH, jh, eq, mC, ad, afb, LH, wD, fU, F, ib, NY, ds, adD, ji, XI, nh, cr, eZ, dq, hp, iV, iR, YZ, qa, LB, bF, Ny, gZ, ei, Lz, i, y, MN); #c1(NLRP3) c2(XP_011542356) c3(NACHT, LRR and PYD domains-containing protein 3 isoform X1) c4(340933) c5(340934, 340935, 340936, 340937, 340938, 340939) c6( ) c7(bol, oO, T, fR, ea, bkF, cl, Ja, xE, fS, dV, iv, K, hH, pk, aa, aW, asD, ih, aoD, eE, cm, axp, Uk, ba, lt, qr, aiM, R, hh, fy, abh, nD, dD, aw, n, amk, NJ, aj, nA, aPb, ai, eW, aDF, ke, adN, h, ey, NX, A, mR, adF, fe, aq, mo, ajz, nN, cM, Zb, bQL, hq, iO, W, bm, hs, fl, ckw, bdR, B, adG, bL, Dw, aAf, fG, yk, jx, ew, cz, azF, v, j, C, V, q, J, o, nX, apJ, aT, qG, aAD, Fg, ae, bf, s, Y, RF, bg, aLJ, r, lo, aDm, Rp, ajl, rR, Uq, wR, dy, yH, fD, aPJ, bQH, jh, eq, mC, ad, afb, LH, wD, fU, F, ib, NY, ds, adD, ji, XI, nh, cr, eZ, dq, hp, iV, iR, YZ, qa, LB, bF, Ny, gZ, ei, Lz, i, y, MN); #c1(NLRP4) c2(XP_006723105) c3(NACHT, LRR and PYD domains-containing protein 4 isoform X2) c4(295007) c5(295008) c6( ) c7(cd, T, q, r, cj); #c1(NLRP4) c2(XP_011524817) c3(NACHT, LRR and PYD domains-containing protein 4 isoform X1) c4( ) c5(319216, 319217, 319218, 319219, 319220) c6( ) c7(cd, T, q, r, cj); #c1(NLRP4) c2(XP_011524818) c3(NACHT, LRR and PYD domains-containing protein 4 isoform X3) c4(319221) c5(319222, 319223, 319224, 319225, 319226, 319227) c6( ) c7(cd, T, q, r, cj); #c1(NLRP5) c2(NP_703148) c3(NACHT, LRR and PYD domains-containing protein 5) c4(246004) c5(246005, 246006, 246007, 246008, 246009, 246010) c6( ) c7(vJ, afc, OX, iI, cj, OM); #c1(NLRP6) c2(NP_001263629) c3(NACHT, LRR and PYD domains-containing protein 6 isoform 2) c4(97316) c5(97317, 97318, 97319, 97320, 97321, 97322) c6( ) c7(bL, aE, qa, cz); #c1(NLRP6) c2(NP_612202) c3(NACHT, LRR and PYD domains-containing protein 6 isoform 1) c4(236196) c5(236197, 236198, 236199, 236200, 236201, 236202) c6( ) c7(bL, aE, qa, cz); #c1(NLRP7) c2(NP_631915) c3(NACHT, LRR and PYD domains-containing protein 7 isoform 1) c4(238304) c5(238305, 238306, 238307, 238308, 238309, 238310, 238311, 238312, 238313, 238314, 238315, 238316, 238317, 238318, 238319, 238320, 238321, 238322, 238323, 238324, 238325, 238326, 238327) c6( ) c7(jv, ZH, CN, vT, aoc, wu, eD, ml, wm, nJ); #c1(NLRP7) c2(XP_006725701) c3(NACHT, LRR and PYD domains-containing protein 7 isoform X2) c4( ) c5(296937, 296938, 296939, 296940, 296941, 296942, 296943, 296944, 296945, 296946, 296947, 296948, 296949, 296950, 296951, 296952, 296953, 296954, 296955, 296956, 296957, 296958, 296959, 296960, 296961, 296962) c6( ) c7(jv, ZH, CN, vT, aoc, wu, eD, ml, wm, nJ); #c1(NLRP7) c2(XP_011545815) c3(NACHT, LRR and PYD domains-containing protein 7 isoform X1) c4(344700, 344701) c5(344702, 344703, 344704, 344705, 344706, 344707, 344708, 344709, 344710, 344711, 344712, 344713, 344714, 344715, 344716, 344717, 344718, 344719, 344720, 344721, 344722, 344723, 344724, 344725, 344726, 344727) c6( ) c7(jv, ZH, CN, vT, aoc, wu, eD, ml, wm, nJ); #c1(NLRP7) c2(XP_011545818) c3(NACHT, LRR and PYD domains-containing protein 7 isoform X3) c4(344728, 344729) c5(344730, 344731, 344732, 344733, 344734, 344735, 344736, 344737, 344738, 344739, 344740, 344741, 344742, 344743, 344744, 344745, 344746, 344747, 344748, 344749, 344750, 344751, 344752, 344753, 344754, 344755, 344756, 344757, 344758, 344759) c6( ) c7(jv, ZH, CN, vT, aoc, wu, eD, ml, wm, nJ); #c1(NLRP8) c2(NP_789781) c3(NACHT, LRR and PYD domains-containing protein 8) c4(252273) c5(252274, 252275, 252276, 252277, 252278, 252279) c6( ) c7(cj); #c1(NLRP9) c2(XP_011525195) c3(NACHT, LRR and PYD domains-containing protein 9 isoform X1) c4(319675, 319676) c5(319677, 319678, 319679, 319680, 319681, 319682, 319683, 319684, 319685, 319686, 319687, 319688) c6( ) c7(cd); #c1(NMB) c2(NP_066563) c3(neuromedin-B isoform 1 preproprotein) c4(207397) c5(207398, 207399, 207400, 207401, 207402) c6( ) c7(eM, anK, v, ci, K, Tp, bz, bJ); #c1(NMB) c2(NP_995580) c3(neuromedin-B isoform 2 preproprotein) c4(263918) c5(263919, 263920, 263921, 263922, 263923, 263924, 263925) c6( ) c7(eM, anK, v, ci, K, Tp, bz, bJ); #c1(NMBR) c2(NP_002502) c3(neuromedin-B receptor) c4(129370) c5(129371, 129372, 129373, 129374, 129375, 129376) c6( ) c7(ft, eM, aW, e, aT, anK, bz); #c1(NME1) c2(NP_000260) c3(nucleoside diphosphate kinase A isoform b) c4( ) c5(5876, 5877, 5878, 5879, 5880) c6( ) c7(T, if, aAx, al, bB, aJN, ci, x, Vp, aE, K, pk, eL, cp, Cn, aW, LZ, g, eF, cm, iP, qJ, eM, nB, iA, iT, aBO, bwr, aep, m, iu, sw, NT, n, aj, kM, fr, qN, Bl, h, dL, asT, dx, CN, PX, aq, aoO, bP, Qu, hL, hR, qF, WL, it, B, cn, fM, dA, AS, KR, Dh, aA, hy, AO, q, J, eA, XX, mj, hW, yg, aT, Fx, ckx, j, ae, af, s, vT, iU, iZ, Y, ev, r, gm, en, cl, apx, t, f, iK, ck, nM, Dy, ig, gX, bt, ao, VB, gx, id, iW, jh, OE, Fb, Gn, iJ, ad, dP, av, hd, Db, yt, vc, lQ, qt, ff, eH, dq, aF, KM, bz, es, bE, bC, y, kS, X); #c1(NME1) c2(NP_937818) c3(nucleoside diphosphate kinase A isoform a) c4(259154, 259155, 259156) c5(259157, 259158, 259159, 259160, 259161, 259162, 259163, 259164, 259165, 259166, 259167, 259168, 259169, 259170, 259171, 259172, 259173, 259174, 259175) c6(259176, 259177) c7(T, if, aAx, al, bB, aJN, ci, x, Vp, aE, K, pk, eL, cp, Cn, aW, LZ, g, eF, cm, iP, qJ, eM, nB, iA, iT, aBO, bwr, aep, m, iu, sw, NT, n, aj, kM, fr, qN, Bl, h, dL, asT, dx, CN, PX, aq, aoO, bP, Qu, hL, hR, qF, WL, it, B, cn, fM, dA, AS, KR, Dh, aA, hy, AO, q, J, eA, XX, mj, hW, yg, aT, Fx, ckx, j, ae, af, s, vT, iL, iZ, Y, ev, r, gm, en, cl, apx, t, f, iK, ck, nM, Dy, ig, gX, bt, ao, VB, gx, id, iW, jh, OE, Fb, Gn, iJ, ad, dP, av, hd, Db, yt, vc, lQ, qt, ff, eH, dq, aF, KM, bz, es, bE, bC, y, kS, X); #c1(NME1-NME2) c2(NP_001018146) c3(NME1-NME2 protein) c4(27452, 27453, 27454, 27455) c5(27456, 27457, 27458, 27459, 27460, 27461, 27462, 27463, 27464, 27465, 27466, 27467, 27468, 27469, 27470, 27471, 27472, 27473, 27474) c6( ) c7(T, dD, dA, KR, ea, eq, fr, qN, q, aT, lQ, K, dx, yo, iI, vT, aW, Y, ff, gm, en, g, t, cm, bz, eD, m, be, B); #c1(NME2) c2(NP_001018148) c3(nucleoside diphosphate kinase B isoform a) c4( ) c5(27475, 27476, 27477, 27478, 27479, 27480, 27481, 27482, 27483, 27484, 27485, 27486, 27487, 27488, 27489, 27490, 27491, 27492, 27493, 27494, 27495, 27496, 27497, 27498, 27499, 27500, 27501, 27502, 27503, 27504, 27505, 27506) c6( ) c7(T, dD, dA, KR, ea, eq, fr, qN, q, ib, aT, lQ, K, af, dx, yo, iU, vT, aW, Y, ff, gm, en, g, t, f, bz, cm, ck, eD, m, be, B); #c1(NME3) c2(NP_002504) c3(nucleoside diphosphate kinase 3 precursor) c4(129377) c5(129378, 129379, 129380, 129381, 129382, 129383) c6( ) c7(d, s, iU, aW, r, cm, q); #c1(NME4) c2(NP_001273364) c3(nucleoside diphosphate kinase, mitochondrial isoform c) c4(104768) c5(104769, 104770, 104771, 104772, 104773, 104774, 104775) c6( ) c7(ck, aA, h, bw, aF, cm, br); #c1(NME4) c2(NP_001273369) c3(nucleoside diphosphate kinase, mitochondrial isoform d) c4(104776, 104777) c5(104778, 104779, 104780, 104781, 104782, 104783, 104784, 104785, 104786, 104787, 104788, 104789, 104790) c6( ) c7(ck, aA, h, bw, af, cm, br); #c1(NME4) c2(NP_005000) c3(nucleoside diphosphate kinase, mitochondrial isoform a precursor) c4(151919, 151920, 151921) c5(151922, 151923, 151924, 151925, 151926, 151927, 151928, 151929, 151930, 151931, 151932, 151933, 151934, 151935, 151936, 151937, 151938, 151939, 151940, 151941) c6( ) c7(ck, aA, h, bw, aF, cm, br); #c1(NME5) c2(NP_003542) c3(nucleoside diphosphate kinase homolog 5) c4(138988) c5(138989, 138990, 138991, 138992, 138993, 138994) c6( ) c7(id, aE); #c1(NME5) c2(XP_011541965) c3(nucleoside diphosphate kinase homolog 5 isoform X2) c4(340454) c5(340455, 340456, 340457, 340458, 340459, 340460) c6( ) c7(id, aE); #c1(NME6) c2(NP_005784) c3(nucleoside diphosphate kinase 6) c4(159314, 159315) c5(159316, 159317, 159318, 159319, 159320, 159321, 159322, 159323, 159324) c6( ) c7(bB, bE, aF); #c1(NME6) c2(XP_005264846) c3(nucleoside diphosphate kinase 6 isoform X1) c4( ) c5(279286) c6( ) c7(bB, bE, aF); #c1(NME7) c2(NP_037462) c3(nucleoside diphosphate kinase 7 isoform a) c4(175633, 175634) c5(175635, 175636, 175637, 175638, 175639, 175640, 175641) c6( ) c7(ac, bE, cz, bB, lB, bA); #c1(NME7) c2(NP_932076) c3(nucleoside diphosphate kinase 7 isoform b) c4( ) c5(258815, 258816, 258817, 258818, 258819) c6( ) c7(ac, bE, cz, bB, lB, bA); #c1(NME8) c2(NP_057700) c3(thioredoxin domain-containing protein 3) c4(192437) c5(192438, 192439, 192440, 192441, 192442, 192443, 192444, 192445, 192446, 192447) c6( ) c7(gU, cky, MG); #c1(NME9) c2(NP_835231) c3(thioredoxin domain-containing protein 6) c4(253400) c5(253401, 253402, 253403, 253404, 253405, 253406, 253407, 253408, 253409, 253410, 253411, 253412, 253413) c6( ) c7(bB, bE, T, af); #c1(NME9) c2(XP_005247486) c3(thioredoxin domain-containing protein 6 isoform X1) c4(267852, 267853) c5(267854, 267855) c6( ) c7(bB, bE, T, af); #c1(NME9) c2(XP_006713679) c3(thioredoxin domain-containing protein 6 isoform X7) c4(287279) c5(287280, 287281, 287282, 287283, 287284, 287285, 287286, 287287, 287288, 287289) c6( ) c7(bB, bE, T, af); #c1(NME9) c2(XP_011511073) c3(thioredoxin domain-containing protein 6 isoform X2) c4( ) c5(301493, 301494, 301495, 301496, 301497, 301498, 301499, 301500, 301501, 301502) c6( ) c7(bB, bE, T, af); #c1(NME9) c2(XP_011511076) c3(thioredoxin domain-containing protein 6 isoform X4) c4( ) c5(301503) c6( ) c7(bB, bE, T, af); #c1(NME9) c2(XP_011511079) c3(thioredoxin domain-containing protein 6 isoform X8) c4(301504) c5(301505, 301506, 301507, 301508, 301509, 301510) c6( ) c7(bB, bE, T, af); #c1(NMI) c2(NP_004679) c3(N-myc-interactor) c4(148785, 148786) c5(148787, 148788, 148789, 148790, 148791, 148792, 148793, 148794, 148795, 148796, 148797, 148798) c6( ) c7(T, aW, Y, aT, n, dL, m, t, eL); #c1(NMNAT1) c2(NP_001284707) c3(nicotinamide mononucleotide adenylyltransferase 1 isoform 1) c4(112378) c5(112379, 112380, 112381, 112382, 112383, 112384, 112385, 112386, 112387, (12388) c6( ) c7(aIF, bfl, W, ckz, aVu, awk, nw, aR); #c1(NMNAT1) c2(NP_001284708) c3(nicotinamide mononucleotide adenylyltransferase 1 isoform 2) c4(112389) c5(112390, 112391, 112392, 112393, 112394, 112395) c6( ) c7(aIF, bfl, W, ckz, aVu, awK, nw, aR); #c1(NMNAT2) c2(NP_055854) c3(nicotinamide mononucleotide adenylyltransferase 2 isoform 1) c4(183927) c5(183928, 183929, 183930, 183931, 183932, 183933) c6( ) c7(cd, Hx, xg, q, r); #c1(NMNAT2) c2(NP_733820) c3(nicotinamide mononucleotide adenylyltransferase 2 isoform 2) c4(247159) c5(247160, 247161, 247162, 247163, 247164, 247165) c6( ) c7(cd, Hx, xg, q, r); #c1(NMNAT3) c2(NP_001186976) c3(nicotinamide mononucleotide adenylyltransferase 3 isoform 2) c4(78908) c5(78909, 78910, 78911, 78912, 78913, 78914) c6( ) c7(hb, kl, EQ); #c1(NMNAT3) c2(NP_835471) c3(nicotinamide mononucleotide adenylyltransferase 3 isoform 1) c4( ) c5(253554, 253555, 253556, 253557, 253558, 253559, 253560, 253561, 253562) c6( ) c7(hb, kl, EQ); #c1(NMNAT3) c2(XP_006713687) c3(nicotinamide mononucleotide adenylyltransferase 3 isoform X2) c4(287290, 287291) c5( ) c6( ) c7(hb, kl, EQ); #c1(NMNAT3) c2(XP_006713692) c3(nicotinamide mononucleotide adenylyltransferase 3 isoform X3) c4(287292, 287293) c5(287294, 287295, 287296, 287297, 287298, 287299, 287300, 287301, 287302, 287303, 287304, 287305, 287306, 287307) c6( ) c7(hb, kl, EQ); #c1(NMNAT3) c2(XP_011511094) c3(nicotinamide mononucleotide adenylyltransferase 3 isoform X4) c4( ) c5(301511, 301512, 301513, 301514, 301515) c6( ) c7(hb, kl, EQ); #c1(NMS) c2(NP_001011717) c3(neuromedin-S precursor) c4(24789) c5(24790, 24791, 24792, 24793, 24794, 24795, 24796, 24797) c6(24798, 24799) c7(T, Ew, kp, hP, bt, fq); #c1(NMT1) c2(NP_066565) c3(glycylpeptide N-tetradecanoyltransferase 1) c4(207410, 207411) c5(207412, 207413, 207414, 207415, 207416, 207417, 207418, 207419, 207420, 207421, 207422, 207423, 207424, 207425, 207426) c6( ) c7(fR, dB, ff, ci, en, X); #c1(NMT1) c2(XP_011523160) c3 (glycylpeptide N-tetradecanoyltransferase 1 isoform X1) c4( ) c5(317695) c6( ) c7(fR, dB, ff, ci, en, X); #c1(NMT2) c2(NP_004799) c3(glycylpeptide N-tetradecanoyltransferase 2) c4(149942, 149943, 149944) c5(149945, 149946, 149947, 149948, 149949, 149950, 149951, 149952, 149953, 149954, 149955, 149956, 149957, 149958) c6( ) c7(dB, ci, UE); #c1(NMT2) c2(XP_006717602) c3(glycylpeptide N-tetradecanoyltransferase 2 isoform X3) c4( ) c5(291245, 291246, 291247, 291248, 291249) c6( ) c7(dB, ci, UE); #c1(NMU) c2(NP_006672) c3(neuromedin-U isoform 1 preproprotein) c4(167020, 167021) c5(167022, 167023, 167024, 167025, 167026, 167027, 167028, 167029, 167030, 167031, 167032, 167033, 167034, 167035, 167036, 167037) c6(167038, 167039, 167040, 167041) c7(T, qQ, v, x, av, aE, aT, dL, ey, eL, af, aW, kp, zE, iK, bz, es, pO, iA, iT, k, bt, it); #c1(NMUR1) c2(NP_006047) c3(neuromedin-U receptor 1) c4(161448) c5(161449, 161450, 161451, 161452, 161453, 161454) c6( ) c7(es, iT, j, iA); #c1(NMUR2) c2(NP_064552) c3(neuromedin-U receptor 2) c4(202969) c5(202970, 202971, 202972, 202973, 202974, 202975) c6( ) c7(v); #c1(NNAT) c2(NP_005377) c3(neuronatin isoform alpha) c4(155567) c5(155568, 155569, 155570, 155571, 155572, 155573, 155574) c6(155575) c7(Fv, T, aj, v, hF, er, J, ft, yT, hn, K, dx, jf, yo, B, oP, s, ff, en, g, cm, ck, eM, U, iu); #c1(NNAT) c2(NP_859017) c3(neuronatin isoform beta) c4(255373) c5(255374, 255375, 255376, 255377, 255378, 255379, 255380) c6( ) c7(Fv, T, aj, v, hF, er, J, ft, yT, hn, K, dx, jf, yo, B, oP, s, ff, en, g, cm, ck, eM, U, iu); #c1(NNMT) c2(NP_006160) c3(nicotinamide N-methyltransferase) c4(162462) c5(162463, 162464, 162465, 162466, 162467, 162468, 162469, 162470, 162471, 162472) c6( ) c7(bM, T, FW, dA, v, uu, fr, q, x, hd, py, Db, hb, ja, aT, dL, K, ux, an, eL, eu, aW, ff, r, en, cm, qJ, nM, ac, nB, lp, wE, go, bm, at, k, anx, bD, B); #c1(NNT) c2(XP_006714524) c3(NAD(P) transhydrogenase, mitochondrial isoform X2) c4(287893) c5(287894, 287895, 287896, 287897, 287898, 287899) c6( ) c7(ak, ckA, v, mf, j, jE, ey, vi); #c1(NNT) c2(XP_011512303) c3(NAD(P) transhydrogenase, mitochondrial isoform X1) c4(302834, 302835, 302836) c5(302837, 302838, 302839, 302840, 302841, 302842, 302843, 302844, 302845, 302846, 302847, 302848, 302849, 302850, 302851, 302852, 302853, 302854, 302855, 302856, 302857, 302858) c6( ) c7(ak, ckA, v, mf, j, jE, ey, vi); #c1(NOA1) c2(NP_115689) c3(nitric oxide-associated protein 1) c4(223557) c5(223558, 223559, 223560, 223561, 223562, 223563) c6( ) c7(W); #c1(NOB1) c2(NP_054781) c3(RNA-binding protein NOB1) c4(177790) c5(177791, 177792, 177793, 177794, 177795, 177796) c6( ) c7(es, T, py, iA, go, fr, fs, eA); #c1(NOBOX) c2(NP_001073882) c3(homeobox protein NOBOX) c4(36280) c5(36281, 36282, 36283, 36284, 36285, 36286) c6( ) c7(ckB, zZ, iI, baZ); #c1(NOBOX) c2(XP_011514093) c3(homeobox protein NOBOX isoform X1) c4(306697) c5(306698, 306699, 306700, 306701, 306702, 306703) c6( ) c7(ckB, zZ, iI, baZ); #c1(NOC3L) c2(NP_071896) c3(nucleolar complex protein 3 homolog) c4(212135, 212136, 212137) c5(212138, 212139, 212140, 212141, 212142, 212143, 212144, 212145, 212146, 212147, 212148, 212149, 212150, 212151, 212152, 212153, 212154, 212155) c6( ) c7(aA, aF, q, ac, r); #c1(NOD1) c2(NP_006083) c3(nucleotide-binding oligomerization domain-containing protein 1) c4(161771) c5(161772, 161773, 161774, 161775, 161776, 161777, 161778, 161779, 161780, 161781, 161782, 161783) c6( ) c7(oO, bL, T, dD, bM, dA, fR, aD, lH, aA, aj, eq, C, V, ai, aR, J, AW, aFl, apJ, qp, aT, qU, h, aaA, uQ, oo, aW, sp, fO, az, r, fe, hN, lo, aF, aB, zC, iV, eE, cT, ac, z, dh, hL, cj, y, X, aDj); #c1(NOD1) c2(XP_011513384) c3(nucleotide-binding oligomerization domain-containing protein 1 isoform X2) c4(305609) c5( ) c6( ) c7(oO, bL, T, dD, bM, dA, fR, aD, lH, aA, aj, eq, C, V, ai, aR, J, AW, aFl, apJ, qp, aT, qU, h, aaA, uQ, oo, aW, sp, fO, az, r, fe, hN, lo, aF, aB, zC, iV, eE, cT, ac, z, dh, hL, cj, y, X, aDj); #c1(NOD1) c2(XP_01513389) c3(nucleotide-binding oligomerization domain-containing protein 1 isoform X7) c4(305610, 305611) c5( ) c6( ) c7(oO, bL, T, dD, bM, dA, fR, aD, lH, aA, aj, eq, C, V, ai, aR, J, AW, aFl, apJ, qp, aT, qU, h, aaA, uQ, oo, aW, sp, fO, az, r, fe, hN, lo, aF, aB, zC, iV, eE, cT, ac, z, dh, hl, cj, y, X, aDj); #c1(NOD1) c2(XP_011513390) c3(nucleotide-binding oligomerization domain-containing protein 1 isoform X8) c4(305612) c5(305613, 305614, 305615, 305616, 305617, 305618, 305619, 305620, 305621, 305622, 305623, 305624, 305625, 305626, 305627, 305628, 305629, 305630, 305631, 305632, 305633, 305634, 305635, 305636, 305637, 305638) c6( ) c7(oO, bL, T, dD, bM, dA, fR, aD, lH, aA, aj, eq, C, V, ai, aR, J, AW, aFl, apJ, qp, aT, qU, h, aaA, uQ, oo, aW, sp, fO, az, r, fe, hN, lo, aF, aB, zC, iV, eE, cT, ac, z, dh, hl, cj, y, X, aDj); #c1(NOD2) c2(NP_071445) c3(nucleotide-binding oligomerization domain-containing protein 2 isoform 1) c4(211716, 211717, 211718) c5(211719, 211720, 211721, 211722, 211723, 211724, 211725, 211726, 211727, 211728, 211729, 211730, 211731) c6( ) c7(oO, bM, T, fR, ea, e, aXb, al, bB, cl, rm, Tz, wa, AW, vV, rw, bW, Yy, Ds, K, hH, amA, Us, YY, aW, qS, az, Rr, hl, ml, ih, hN, dg, zC, pL, ve, uC, eE, fT, lt, gt, Jr, aFO, qr, cj, uo, k, ckG, aSs, qR, xN, ajy, dD, Ht, sQ, ckE, n, BH, aj, oY, qW, ai, aPb, KL, i, afn, ju, cU, lG, h, dL, ey, aNj, ux, bEg, NX, OV, mR, A, aiB, sp, bPr, ge, fe, nN, cM, acD, bxB, Wt, aBs, ar, acC, hk, ckD, hL, aum, hs, uz, ut, at, SZ, JM, aDE, B, bL, Dw, cai, AL, wL, fG, YN, aA, cz, baK, j, C, V, q, J, cjf, ga, nX, apJ, ld, aT, qU, yu, cy, XU, an, bA, azU, Y, aZs, r, aMA, lo, ahl, t, Ym, ck, ajl, cT, ig, WP, gX, wR, dy, bt, xb, sY, aCA, aD, iW, axd, dG, Gn, eq, hF, wD, av, biq, l, bNN, oz, Dq, vB, ji, bVZ, lQ, ckC, NM, aAy, uQ, bRb, aFP, ckF, baT, aLI, aFt, cr, dN, aFr, eZ, aF, hp, Sm, iV, bz, dr, blZ, bFf, gk, bE, z, eB, wz, Xs, lM, hE, ra, X, y); #c1(NOD2) c2(XP_011521561) c3(nucleotide-binding oligomerization domain-containing protein 2 isoform X4) c4(316293) c5(316294, 316295, 316296, 316297, 316298, 316299) c6( ) c7(oO, bM, T, fR, ea, e, aXb, al, bB, cL, rm, Tz, wa, AW, vV, rw, bW, Yy, Ds, K, hH, amA, Us, YY, aW, qS, az, Rr, hl, mQ, ih, hN, dg, zC, pL, ve, uC, eE, fT, lt, gt, Jr, aFO, qr, cj, uo, k, ckG, aSs, qR, xN, ajy, dD, Ht, sQ, ckE, n, BH, aj, oY, qW, ai, aPb, KL, i, afn, ju, cU, lG, h, dL, ey, aNj, ux, bEg, NX, OV, mR, A, aiB, sp, bPr, ge, fe, nN, cM, acD, bxB, Wt, aBs, ar, acC, hk, ckD, hL, aum, hs, uz, ut, at, SZ, JM, aDE, B, bL, Dw, cai, AL, wL, fG, YN, aA, cz, baK, j, C, V, q, J, cjf, ga, nX, apJ, ld, aT, qU, yu, cy, XU, an, bA, azU, Y, aZs, r, aMA, lo, ahl, t, Ym, ck, ajl, cT, ig, WP, gX, wR, dy, bt, xb, sY, aCA, aD, iW, axd, dG, Gn, eq, hF, wD, av, biq, I, bNN, oz, Dq, vB, ji, bVZ, lQ, ckC, NM, aAy, ul, bRb, aFP, ckF, baT, aLI, aFt, cr, dN, aFr, eZ, aF, hp, Sm, iV, bz, dr, blZ, bFf, gk, bE, z, eB, wz, Xs, lM, hE, ra, X, y); #c1(NOD2) c2(XP_011521563) c3(nucleotide-binding oligomerization domain-containing protein 2 isoform X7) c4( ) c5(316300, 316301, 316302, 316303, 316304) c6( ) c7(oO, bM, T, fR, ea, e, aXb, al, bB, cL, rm, Tz, wa, AW, wV, rw, bW, Yy, Ds, K, hH, amA, Us, YY, aW, qS, az, Rr, hl, mQ, ih, hN, dg, zC, pL, ve, uC, eE, fT, lt, gt, Jr, aFO, qr, cj, uo, k, ckG, aSs, qR, xN, ajy, dD, Ht, sQ, ckE, n, BH, aj, oY, qW, ai, aPb, KL, i, afn, ju, cU, lG, h, dL, ey, aNj, ux, bEg, NX, OV, mR, A, aiB, sp, bPr, ge, fe, nN, cM, acD, bxB, Wt, aBs, ar, acC, hk, ckD, hL, aum, hs, uz, ut, at, SZ, JM, aDE, B, bL, Dw, cai, AL, wL, fG, YN, aA, cz, baK, j, C, V, q, J, cjf, ga, nX, apJ, ld, aT, qU, yu, cy, XU, an, bA, azU, Y, aZs, r, aMA, lo, ahl, t, Ym, ck, ajl, cT, ig, WP, gX, wR, dy, bt, xb, sY, aCA, aD, iW, axd, dG, Gn, eq, hF, wD, av, biq, l, bNN, oz, Dq, vB, ji, bVZ, lQ, ckC, NM, aAy, ul, bRb, aFP, ckF, baT, aLI, aFt, cr, dN, aFr, eZ, aF, hp, Sm, iV, bz, dr, blZ, bFf, gk, bE, z, eB, wz, Xs, lM, hE, ra, X, y); #c1(NODAL) c2(NP_060525) c3(nodal homolog precursor) c4(196370, 196371) c5(196372, 196373, 196374, 196375, 196376, 196377, 196378, 196379, 196380, 196381, 196382, 196383, 196384, 196385, 196386, 196387, 196388) c6( ) c7(T, BN, ne, aW, Y, arR, hy, aDR, j, eA, ZW, ckH, ae, B, ad); #c1(NOG) c2(NP_005441) c3(noggin precursor) c4(156199) c5(156200, 156201, 156202, 156203, 156204, 156205) c6( ) c7(atx, T, dA, rV, ht, cz, cR, v, zu, ad, bv, ckN, iI, btl, aT, Ne, h, Nc, K, hu, ZW, a, bA, je, ckl, aW, Y, ate, apn, nY, ckM, bBd, ckL, agj, ckJ, zZ, ckQ, bz, bvU, ckK, zV, cm, atm, iM, BL, NL, w, aVv, pg, aS, dy, bkh, VK, B, atd); #c1(NOL11) c2(NP_001290201) c3(nucleolar protein 11 isoform 2) c4( ) c5(115141, 115142, 115143, 115144, 115145) c6( ) c7(aOE); #c1(NOL11) c2(NP_056277) c3(nucleolar protein 11 isoform 1) c4(186891, 186892) c5(186893, 186894, 186895, 186896, 186897, 186898, 186899) c6( ) c7(aOE); #c1(NOL3) c2(NP_001263236) c3(nucleolar protein 3 isoform MYP) c4(97073) c5(97074, 97075, 97076, 97077, 97078, 97079, 97080, 97081) c6( ) c7(T, fM, id, aw, afk, eX, jh, aA, bB, j, ci, fr, afl, AO, jw, q, aT, h, eV, vc, K, B, aW, Y, r, aF, BG, iV, cm, lk, kC, xw, ck, es, sS, iA, bE, mx, Ei, ckP, y, bt, X); #c1(NOL3) c2(NP_001263248) c3(nucleolar protein 3 isoform D) c4( ) c5(97087, 97088) c6( ) c7(T, fM, id, aw, afk, eX, jh, aA, bB, j, ci, fr, afl, AO, jw, q, aT, h, eV, vc, K, B, aW, Y, r, aF, BG, iV, cm, lk, kC, xw, ck, es, sS, iA, bE, mx, Ei, ckP, y, bt, X); #c1(NOL4) c2(NP_001185475) c3(nucleolar protein 4 isoform 2) c4(75705, 75706) c5( ) c6( ) c7(if, T, fq, ac, qN); #c1(NOL4) c2(NP_001185476) c3(nucleolar protein 4 isoform 3) c4(75707) c5(75708, 75709, 75710, 75711, 75712, 75713) c6( ) c7(if, T, fq, ac, qN); #c1(NOL4) c2(NP_001185477) c3(nucleolar protein 4 isoform 4) c4(75714) c5(75715, 75716, 75717, 75718, 75719, 75720) c6( ) c7(if, T, fq, ac, qN); #c1(NOL4) c2(NP_001185478) c3(nucleolar protein 4 isoform 5) c4( ) c5(75721, 75722, 75723, 75724, 75725) c6( ) c7(if, T, fq, ac, qN); #c1(NOL4) c2(NP_001269456) c3(nucleolar protein 4 isoform 6) c4( ) c5(101696, 101697, 101698, 101699, 101700, 101701, 101702, 101703, 101704, 101705, 101706) c6( ) c7(if, T, fq, ac, qN); #c1(NOL4) c2(NP_003778) c3(nucleolar protein 4 isoform 1) c4(140762) c5(140763, 140764, 140765, 140766, 140767, 140768) c6( ) c7(if, T, fq, ac, qN); #c1(NOL4) c2(XP_006722627) c3(nucleolar protein 4 isoform X3) c4( ) c5(294786) c6( ) c7(if, T, fq, ac, qN); #c1(NOL4) c2(XP_006722629) c3(nucleolar protein 4 isoform X5) c4( ) c5(294787) c6( ) c7(if, T, fq, ac, qN); #c1(NOL4) c2(XP_011524542) c3(nucleolar protein 4 isoform X4) c4(319146) c5( ) c6( ) c7(if, T, fq, ac, qN); #c1(NOL6) c2(NP_075068) c3(nucleolar protein 6 alpha isoform) c4(213662, 213663, 213664, 213665, 213666, 213667) c5(213668, 213669, 213670, 213671, 213672, 213673, 213674, 213675, 213676, 213677, 213678, 213679, 213680, 213681, 213682, 213683, 213684, 213685, 213686, 213687, 213688, 213689, 213690, 213691, 213692, 213693, 213694, 213695, 213696, 213697, 213698, 213699, 213700, 213701, 213702, 213703) c6( ) c7(aw); #c1(NOL6) c2(NP_631981) c3(nucleolar protein 6 gamma isoform) c4(238418) c5(238419, 238420, 238421, 238422, 238423, 238424) c6( ) c7(aw); #c1(NOL8) c2(NP_001243323) c3(nucleolar protein 8 isoform b) c4(90409) c5( ) c6( ) c7(T, aF, aA); #c1(NOL8) c2(NP_060418) c3(nucleolar protein 8 isoform a) c4(195858, 195859) c5(195860, 195861, 195862, 195863, 195864, 195865, 195866, 195867, 195868, 195869, 195870, 195871, 195872, 195873, 195874, 195875, 195876, 195877, 195878, 195879, 195880, 195881, 195882) c6( ) c7(T, aF, aA); #c1(NOL8) c2(XP_006717231) c3(nucleolar protein 8 isoform X3) c4(290812, 290813) c5(290814, 290815, 290816, 290817, 290818, 290819) c6( ) c7(T, aF, aA); #c1(NOL8) c2(XP_011517128) c3(nucleolar protein 8 isoform X4) c4(310637, 310638, 310639) c5(310640, 310641, 310642, 310643, 310644, 310645, 310646, 310647, 310648, 310649, 310650, 310651, 310652, 310653, 310654, 310655, 310656, 310657, 310658, 310659, 310660, 310661, 310662) c6( ) c7(T, aF, aA); #c1(NOLC1) c2(NP_001271317) c3(nucleolar and coiled-body phosphoprotein 1 isoform 1) c4(103883) c5(103884, 103885, 103886, 103887, 103888, 103889) c6( ) c7(T, if, jh, aA, ci, qN, q, ad, ft, lg, aT, K, ae, af, aJE, d, aW, ff, r, aF, en, bP, eD, m, B, L); #c1(NOLC1) c2(NP_004732) c3(nucleolar and coiled-body phosphoprotein 1 isoform 2) c4(149158) c5(149159, 149160, 149161, 149162, 149163, 149164) c6( ) c7(T, if, jh, aA, ci, qN, q, ad, ft, lg, aT, K, ae, af, aJE, d, aW, ff, r, aF, en, bP, eD, m, B, L); #c1(NOMI) c2(NP_612409) c3(nucleolar MIF4G domain-containing protein 1) c4(236414) c5(236415, 236416, 236417, 236418, 236419, 236420) c6( ) c7(ac); #c1(NOMO1) c2(NP_055102) c3(nodal modulator 1 precursor) c4(178956) c5(178957, 178958, 178959, 178960, 178961, 178962) c6( ) c7(bt, dA, kH); #c1(NONO) c2(NP_001138881) c3(non-POU domain-containing octamer-binding protein isoform 1) c4(57997, 57998, 57999, 58000, 58001, 58002, 58003) c5(58004, 58005, 58006, 58007, 58008, 58009, 58010, 58011, 58012, 58013, 58014, 58015, 58016, 58017, 58018, 58019, 58020, 58021, 58022, 58023, 58024, 58025, 58026, 58027, 58028, 58029, 58030, 58031, 58032, 58033, 58034, 58035, 58036, 58037, 58038, 58039, 58040, 58041, 58042, 58043) c6( ) c7(Y, ev, r, aT, ci, K, rP, q); #c1(NONO) c2(NP_001138882) c3(non-POU domain-containing octamer-binding protein isoform 2) c4( ) c5(58044, 58045, 58046, 58047, 58048, 58049, 58050, 58051, 58052, 58053, 58054) c6( ) c7(Y, ev, r, aT, ci, K, rP, q); #c1(NOP10) c2(NP_061118) c3(H/ACA ribonucleoprotein complex subunit 3) c4(199200) c5(199201, 199202, 199203, 199204, 199205, 199206) c6( ) c7(sb, cju, cjV); #c1(NOP14) c2(NP_001278908) c3(nucleolar protein 14 isoform 2) c4(110861) c5(110862, 110863, 110864, 110865, 110866, 110867) c6( ) c7(aE); #c1(NOP14) c2(NP_003694) c3(nucleolar protein 14 isoform 1) c4(140208, 140209) c5(140210, 140211, 140212, 140213, 140214, 140215, 140216, 140217, 140218, 140219, 140220, 140221, 140222, 140223, 140224, 140225) c6( ) c7(aE); #c1(NOP16) c2(NP_001243468) c3(nucleolar protein 16 isoform 1) c4(90887) c5(90888, 90889, 90890, 90891, 90892, 90893) c6( ) c7(aW, aq, aT); #c1(NOP16) c2(NP_001243469) c3(nucleolar protein 16 isoform 2) c4(90894) c5(90895, 90896, 90897, 90898, 90899, 90900) c6( ) c7(aW, aq, aT); #c1(NOP16) c2(NP_001278234) c3(nucleolar protein 16 isoform 4) c4(109522) c5(109523, 109524, 109525, 109526, 109527, 109528) c6( ) c7(aW, aq, aT); #c1(NOP16) c2(NP_057475) c3(nucleolar protein 16 isoform 3) c4(191136) c5(191137, 191138, 191139, 191140, 191141, 191142) c6( ) c7(aW, aq, aT); #c1(NOP2) c2(NP_001028886) c3(probable 28S rRNA (cytosine(4447)-C(5)-methyltransferase isoform 1) c4(30686) c5(30687, 30688, 30689, 30690, 30691, 30692, 30693, 30694, 30695, 30696) c6( ) c7(T, aE, AO, qt, aW, r, aT, ci, nA, zy, hF, eA); #c1(NOP2) c2(NP_001245238) c3(probable 28S rRNA (cytosine(4447)-C(5)-methyltransferase isoform 3) c4(92765) c5(92766, 92767, 92768, 92769, 92770, 92771) c6( ) c7(T, aE, AO, qt, aW, r, aT, ci, nA, zy, hF, eA); #c1(NOP2) c2(NP_001245239) c3 (probable 28S rRNA (cytosine(4447)-C(5)-methyltransferase isoform 4) c4(92772) c5(92773, 92774, 92775, 92776, 92777, 92778) c6( ) c7(T, aE, AO, qt, aW, r, aT, ci, nA, zy, hF, eA); #c1(NOP2) c2(XP_005253748) c3(probable 28S rRNA (cytosine(4447)-C(5))-methyltransferase isoform X1) c4(272630) c5(272631, 272632, 272633, 272634, 272635, 272636, 272637, 272638, 272639, 272640) c6( ) c7(T, aE, AO, qt, aW, r, aT, ci, nA, zy, hF, eA); #c1(NOP56) c2(NP_006383) c3(nucleolar protein 56) c4(164525, 164526, 164527, 164528) c5(164529, 164530, 164531, 164532, 164533, 164534, 164535, 164536, 164537, 164538, 164539, 164540, 164541, 164542, 164543, 164544, 164545, 164546, 164547, 164548, 164549, 164550, 164551, 164552) c6( ) c7(ky, kB, OI, jh, aA, rf, aIQ); #c1(NOP9) c2(NP_777573) c3(nucleolar protein 9 isoform 1) c4(251171) c5(251172, 251173, 251174, 251175, 251176, 251177) c6( ) c7(aT); #c1(NOP9) c2(XP_011534828) c3(nucleolar protein 9 isoform X2) c4(331330) c5(331331, 331332, 331333, 331334, 331335, 331336) c6( ) c7(aT); #c1(NOS1) c2(NP_000611) c3(nitric oxide synthase, brain isoform 1) c4(13743, 13744, 13745, 13746, 13747) c5(13748, 13749, 13750, 13751, 13752, 13753, 13754, 13755, 13756, 13757, 13758, 13759, 13760, 13761, 13762, 13763, 13764, 13765, 13766, 13767, 13768) c6( ) c7(oO, tJ, T, bM, ea, aqo, e, al, yY, gf, ur, ci, HH, Jt, aY, aR, ip, aE, bWj, dV, Ds, K, qD, xa, aW, mQ, vs, Ku, Eg, g, cm, bw, bdc, y, eM, lp, oy, mx, vy, cj, bjL, sv, pw, act, eg, fy, ul, dD, xJ, aj, fr, ct, eh, cd, ai, cc, h, lG, he, A, sp, EU, bzu, Og, cA, dB, at, cB, bL, Dw, iH, eR, bBr, hx, cz, ckQ, v, j, C, il, V, eA, J, uT, WY, sy, qK, HC, MI, aT, cy, bK, ahg, bq, an, rz, ae, bPQ, af, eu, afC, Y, ev, aPf, HL, en, bN, bNB, ck, Qi, pH, cT, iM, ac, c, ciO, bt, ao, bOo, vq, am, akd, MH, qb, iW, uG, ckR, jh, bS, ah, oH, dG, uu, bzq, eq, ad, dP, av, HF, ot, oj, vc, vP, MG, d, je, aCt, ff, Of, eZ, iV, Z, jb, qa, es, gk, aaG, wz, sG, aGg, lC, vt, xg, cbO, rk); #c1(NOS1) c2(NP_001191142) c3(nitric oxide synthase, brain isoform 3) c4( ) c5(81073, 81074, 81075, 81076, 81077, 81078) c6( ) c7(oO, tJ, T, bM, ea, aqo, e, al, yY, gf, ur, ci, HH, Jt, aY, aR, ip, aE, bWj, dV, Ds, K, qD, xa, aW, mQ, vs, Ku, Eg, g, cm, bw, bdc, y, eM, lp, oy, mx, vy, cj, bjL, sv, pw, act, eg, fy, uL, dD, xJ, aj, fr, ct, eh, cd, ai, cc, h, lG, he, A, sp, EU, bzu, Og, cA, dB, at, cB, bL, Dw, iH, eR, bBr, hx, cz, ckQ, v, j, C, il, V, eA, J, uT, WY, sy, qK, HC, MI, aT, cy, bK, ahg, bq, an, rz, ae, bPQ, af, eu, afC, Y, ev, aPf, HL, en, bN, bNB, ck, Qi, pH, cT, iM, ac, c, ciO, bt, ao, bOo, vq, am, akd, MH, qb, iW, uG, ckR, jh, bS, ah, oH, dG, uu, bzq, eq, ad, dP, av, HF, ot, oj, vc, vP, MG, d, je, aCt, ff, Of, eZ, iV, Z, jb, qa, es, gk, aaG, wz, sG, aGg, lC, vt, xg, cbO, rk); #c1(NOS1) c2(NP_001191147) c3(nitric oxide synthase, brain isoform 2) c4(81079, 81080) c5(81081, 81082, 81083, 81084, 81085, 81086, 81087, 81088, 81089, 81090, 81091, 81092, 81093) c6( ) c7(oO, tJ, T, bM, ea, aqo, e, al, yY, gf, ur, ci, HH, Jt, aY, aR, ip, aE, bWj, dV, Ds, K, qD, xa, aW, mQ, vs, Ku, Eg, g, cm, bw, bdc, y, eM, lp, oy, mx, vy, cj, bjL, sv, pw, act, eg, fy, ul, dD, xJ, aj, fr, ct, eh, cd, ai, cc, h, lG, he, A, sp, EU, bzu, Og, cA, dB, at, cB, bL, Dw, iH, eR, bBr, hx, cz, ckQ, v, j, C, iI, V, eA, J, uT, WY, sy, qK, HC, MI, aT, cy, bK, ahg, bq, an, rz, ae, bPQ, af, eu, afC, Y, ev, aPf, HL, en, bN, bNB, ck, Qi, pH, cT, iM, ac, c, ciO, bt, ao, bOo, vq, am, akd, MH, qb, iW, uG, ckR, jh, bS, ah, oH, dG, uu, bzq, eq, ad, dP, av, HF, ot, oj, vc, vP, MG, d, je, aCt, ff, Of, eZ, iV, Z, jb, qa, es, gk, aaG, wz, sG, aGg, lC, vt, xg, cbO, rk); #c1(NOS1AP) c2(NP_001119532) c3(carboxyl-terminal PDZ ligand of neuronal nitric oxide synthase protein isoform 2) c4(44141) c5(44142, 44143, 44144, 44145, 44146, 44147) c6( ) c7(bL, dD, tS, atB, ea, e, My, aw, EQ, rf, tc, aj, bS, j, ct, eq, abQ, cd, J, no, aT, By, bK, K, he, HO, mu, uX, aW, EZ, pL, Z, ckS, Ei, gZ, gq); #c1(NOS1AP) c2(NP_001158229) c3(carboxyl-terminal PDZ ligand of neuronal nitric oxide synthase protein isoform 3) c4(63482) c5(63483, 63484, 63485, 63486, 63487, 63488, 63489, 63490) c6( ) c7(bL, dD, tS, atB, ea, e, My, aw, EQ, rf, tc, aj, bS, j, ct, eq, abQ, cd, J, no, aT, By, bK, K, he, HO, mu, uX, aW, EZ, pL, Z, ckS, Ei, gZ, gq); #c1(NOS1AP) c2(NP_055512) c3(carboxyl-terminal PDZ ligand of neuronal nitric oxide synthase protein isoform 1) c4(181621) c5(181622, 181623, 181624, 181625, 181626, 181627) c6( ) c7(bL, dD, tS, atB, ea, e, My, aw, EQ, rf, tc, aj, bS, j, ct, eq, abQ, cd, J, no, aT, By, bK, K, he, HO, mu, uX, aW, EZ, pL, Z, ckS, Ei, gZ, gq); #c1(NOS2) c2(NP_000616) c3(nitric oxide synthase, inducible) c4(13941, 13942, 13943) c5(13944, 13945, 13946, 13947, 13948, 13949, 13950, 13951, 13952, 13953, 13954, 13955, 13956, 13957, 13958, 13959, 13960, 13961, 13962) c6( ) c7(oO, bM, G, T, lD, xS, fR, hV, ea, atj, e, al, eb, bB, zh, ci, qj, bh, aY, sD, Tz, aR, ip, Bc, dV, us, Ds, K, GO, bah, zy, H, hH, eL, bc, VF, bzH, aa, ly, dH, Jl, aW, bn, aoo, wf, hl, LZ, kt, hN, g, Eg, aFU, hJ, uC, cm, bw, ln, bdc, y, fT, eM, iA, lp, aDs, Jp, oy, mx, vy, cj, Gs, aVm, dW, mk, m, pw, eg, hh, aTW, qR, dD, pG, aw, ht, NT, fN, n, BH, xJ, aj, ct, uE, fr, eh, cd, zo, ai, eW, ckT, jz, cc, RN, ju, kH, cU, lG, h, dL, vd, ey, hB, he, aDp, pl, wC, A, mR, sp, EU, bzu, Ff, ge, fQ, QP, aq, fe, Og, aGq, jh, dw, cM, lx, kT, W, bP, dB, wk, lf, JM, bm, at, E, apY, aAl, qF, amb, fL, Pr, B, cB, bL, Dw, eR, jp, uv, Ju, jx, rf, aA, cR, tc, cz, hy, j, v, C, OY, wo, yV, V, wy, q, J, ga, uT, hb, jZ, qp, qK, aT, Di, qG, bK, ahg, bq, XZ, an, ae, bsd, bPQ, af, CB, dL, ak, eu, afC, zH, Y, ev, fO, r, aPf, lo, en, aaO, t, kC, iK, pb, bN, ck, Qi, akF, wg, Dy, c, ac, lI, ig, nM, iM, hO, ro, cil, dy, Da, gT, bt, aen, ao, bOo, sY, ciH, wi, aD, dM, iW, gN, ckR, aZH, OR, auq, bS, oH, jE, uu, aXD, dG, eq, wV, ad, wD, LH, dP, av, F, yt, ds, lC, oW, Os, vw, io, JK, ji, jo, JS, MG, sj, ul, NP, uX, je, lR, aZl, ff, Of, cr, eZ, hv, aF, hp, iV, Z, bz, qa, ek, adA, aDN, es, D, gk, pu, TA, axs, bF, bE, Ny, dO, X, uR, wz, aDB, gZ, aS, ME, ei, hE, vt, ne, rk, pC); #c1(NOS3) c2(NP_000594) c3(nitric oxide synthase, endothelial isoform 1) c4(13516, 13517) c5(13518, 13519, 13520, 13521, 13522, 13523, 13524, 13525, 13526, 13527, 13528, 13529, 13530, 13531, 13532, 13533, 13534) c6( ) c7(T, ea, bO, ci, aY, xy, dV, HR, hH, aa, ly, ahP, nY, GR, ba, lp, wE, k, lS, aNn, uL, aEw, ht, cQ, aj, cd, sW, lG, bxi, aaL, ey, sp, vu, fQ, fe, ayt, dB, E, Dp, aA, cz, nS, q, QI, cy, sR, Cy, dL, eu, bA, bf, bg, aeS, pb, rR, OW, tW, ac, mG, bcs, hr, ao, arS, iW, uG, mw, dG, uu, fu, ad, HF, yt, vJ, Aa, lQ, vP, WZ, ZE, ue, cH, cW, ag, dX, lC, ra, rk, lD, bM, ckX, e, ur, ip, x, Na, aif, bc, dH, hI, fH, as, ckU, mx, vy, bjL, tG, wx, dD, gU, aJr, md, Mo, ec, bmo, aDX, hi, MX, A, aRa, ty, sE, cA, c, at, ut, fl, sa, sZ, B, mr, wL, fF, v, V, UE, WY, rS, MI, ae, HL, t, sk, acp, wO, hO, kV, HQ, dM, OR, eq, BY, LA, hD, vc, JK, ckW, sj, wc, Z, HI, D, gk, yE, wz, gZ, gB, Tw, y, aax, hv, fR, al, gf, wa, lu, Bc, M, K, wt, eL, wf, g, aBa, uC, Gs, U, uo, fC, m, P, sQ, sw, aw, aCQ, eh, fK, axc, Lv, Ff, wd, cM, kT, wk, mg, gv, gg, Pr, oe, jx, wo, j, eA, J, nX, Nc, bq, bPQ, kl, Y, ckV, r, iK, bmS, sn, c, hc, dh, wR, cS, dy, fD, gT, gx, sY, am, akd, aZ, bS, ah, gb, Jm, fU, dP, ji, uX, lR, cr, qa, adA, cgA, ckY, ei, vt, st, wH, acz, eb, dY, iI, dQ, eN, hm, aAu, LC, aW, aDW, hJ, eM, wB, bWG, nw, qR, aEj, tp, wh, ez, ct, cc, h, jy, dL, vx, Gc, wC, aq, mo, dw, lx, Vy, FV, bm, te, bD, um, bl, eR, dk, uv, sM, cR, C, acj, uT, qp, aT, vH, bK, yb, an, af, ak, qw, ND, aaE, f, bNB, ajl, wg, nM, pZ, mf, lJ, hS, qX, uq, Zl, qb, vr, oH, av, ib, sO, ts, jo, bjP, uQ, eZ, aF, eK, apM, z, uR, aS, tr, tP, us); #c1(NOS3) c2(NP_00153581) c3(nitric oxide synthase, endothelial isoform 2) c4(60686) c5(60687) c6( ) c7(T, ea, bO, ci, aY, xy, dV, HR, hH, aa, ly, ahP, nY, GR, ba, lp, wE, k, lS, aNn, uL, aEw, ht, cQ, aj, cd, sW, lG, bxi, aaL, ey, sp, vu, fQ, fe, ayt, dB, E, Dp, aA, cz, nS, q, QI, cy, sR, Cy, dL, eu, bA, bf, bg, aeS, pb, rR, OW, tW, ac, mG, bcs, hr, ao, arS, iW, uG, mw, dG, uu, fu, ad, HF, yt, vJ, Aa, lQ, vP, WZ, ZE, ue, cH, cW, ag, dX, lC, ra, rk, lD, bM, ckX, e, ur, ip, x, Na, aif, bc, dH, hl, fH, as, ckU, mx, vy, bjL, tG, wx, dD, gU, aJr, md, Mo, ec, bmo, aOX, hi, MX, A, aRa, ty, sE, cA, cO, at, ut, fl, sa, sZ, B, mr, wL, fF, v, V, UE, WY, rS, MI, ae, HL, t, sk, acp, wO, hO, kV, HQ, dM, OR, eq, BY, LA, hD, vc, JK, ckW, sj, wc, Z, HI, D, gk, yE, wz, gZ, gB, Tw, y, aax, hv, FR, al, gf, wa, lu, Bc, M, K, wt, eL, wf, g, aBa, uC, Gs, U, uo, fC, m, P, sQ, sw, aw, aCQ, eh, fK, axc, Lv, Ff, wd, cM, kT, wk, mg, gv, gg, Pr, oe, jx, wo, j, eA, J, nX, Nc, bq, bPQ, kl, Y, ckV, r, iK, bmS, sn, c, hc, dh, wR, cS, dy, fD, gT, gx, sY, am, akd, aZ, bS, ah, gb, Jm, fU, dP, ji, uX, lR, cr, qa, adA, cgA, ckY, ei, vt, st, wH, acz, eb, dY, iI, dQ, eN, hm, aAu, LC, aW, aOW, hJ, eM, wB, bWG, nw, qR, aEj, tp, wh, ez, ct, cc, h, jy, dL, vx, Gc, wC, aq, mo, dw, lx, Vy, FV, bm, te, bD, um, bL, eR, dk, uv, sM, cR, C, acj, uT, qp, aT, vH, bK, yb, an, af, ak, qw, ND, aaE, f, bNB, ajl, wg, nM, pZ, mf, lJ, hS, qX, uq, Zl, qb, vr, oH, av, ib, sO, ts, jo, bjP, uQ, eZ, aF, ek, apM, z, uR, aS, tr, tP, us); #c1(NOS3) c2(NP_001153582) c3(nitric oxide synthase, endothelial isoform 3) c4(60688, 60689) c5(60690) c6( ) c7(T, ea, bO, ci, aY, xy, dV, HR, hH, aa, ly, ahP, nY, GR, ba, lp, wE, k, lS, aNn, uL, aEw, ht, cQ, aj, cd, sW, lG, bxi, aaL, ey, sp, vu, fQ, fe, ayt, dB, E, Dp, aA, cz, nS, q, QI, cy, sR, Cy, dL, eu, bA, bf, bg, aeS, pb, rR, OW, tW, ac, mG, bcs, hr, ao, arS, iW, uG, mw, dG, uu, fu, ad, HF, yt, vJ, Aa, lQ, vP, WZ, ZE, ue, cH, cW, ag, dX, lC, ra, rk, lD, bM, ckX, e, ur, ip, x, Na, aif, bc, dH, hl, fH, as, ckU, mx, vy, bjL, tG, wx, dD, gU, aJr, md, Mo, ec, bmo, aDX, hi, MX, A, aRa, ty, sE, cA, cO, at, ut, fl, sa, sZ, B, mr, wL, fF, v, V, UE, WY, rS, MI, ae, HL, t, sk, acp, wO, hO, kV, HQ, dM, OR, eq, BY, LA, hD, vc, JK, ckW, sj, wc, Z, HI, D, gk, yE, wz, gZ, gB, Tw, y, aax, hv, fR, al, gf, wa, lu, Bc, M, K, wt, eL, wf, g, aBa, uC, Gs, U, uo, fC, m, P, s, sw, aw, aCQ, eh, fK, axc, Lv, Ff, wd, cM, kT, wk, mg, gv, gg, Pr, oe, jx, wo, j, eA, J, nX, Nc, bq, bPQ, kl, Y, ckV, r, iK, bmS, sn, c, hc, dh, wR, cS, dy, fD, gT, gx, sY, am, akd, aZ, bS, ah, gb, Jm, fU, dP, ji, uX, IR, cr, qa, adA, cgA, ckY, ei, vt, st, wH, acz, eb, dY, iI, dQ, eN, hm, aAu, LC, aW, aOW, hJ, eM, wB, bWG, nw, qR, aEj, tp, wh, ez, ct, cc, h, jy, dL, vx, Gc, wC, aq, mo, dw, lx, Vy, FV, bm, te, bD, um, bL, eR, dk, uv, sM, cR, C, acj, uT, qp, aT, vH, bK, yb, an, af, ak, qw, ND, aaE, f, bNB, ajl, wg, nM, pZ, mf, lJ, hS, qX, uq, Zl, qb, vr, oH, av, ib, sO, ts, jo, bjP, uQ, eZ, aF, ek, apM, z, uR, aS, tr, tP, us); #c1(NOS3) c2(NP_001153583) c3(nitric oxide synthase, endothelial isoform 4) c4(60691) c5(60692) c6( ) c7(T, ea, bO, ci, aY, xy, dV, HR, hH, aa, ly, ahP, nY, GR, ba, lp, wE, k, lS, aNn, uL, aEw, ht, cl, aj, cd, sW, lG, bxi, aaL, ey, sp, vu, fQ, fe, ayt, dB, E, Dp, aA, cz, nS, q, QI, cy, sR, Cy, dL, eu, bA, bf, bg, aeS, pb, rR, OW, tW, ac, mG, bcs, hr, ao, arS, iW, uG, mw, dG, uu, fu, ad, HF, yt, vJ, Aa, lQ, vP, WZ, ZE, ue, cH, cW, ag, dX, lC, ra, rk, lD, bM, ckX, e, ur, ip, x, Na, aif, bc, dH, hl, fH, as, ckU, mx, vy, bjL, tG, wx, dD, gU, aJr, md, Mo, ec, bmo, aDX, hi, MX, A, aRa, ty, sE, cA, cO, at, ut, fl, sa, sZ, B, mr, wL, fF, v, V, UE, WY, rS, MI, ae, HL, t, sk, acp, wO, hO, kV, HQ, dM, OR, eq, BY, LA, hD, vc, JK, ckW, sj, wc, Z, HI, D, gk, yE, wz, gZ, gB, Tw, y, aax, hv, FR, al, gf, wa, lu, Bc, M, K, wt, eL, wf, g, aBa, uC, Gs, U, uo, fC, m, P, sQ, sw, aw, aCQ, eh, fK, axc, Lv, Ff, wd, cM, kT, wk, mg, gv, gg, Pr, oe, jx, wo, j, eA, J, nX, Nc, bq, bPQ, kl, Y, ckV, r, iK, bmS, sn, c, hc, dh, wR, cS, dy, fD, gT, gx, sY, am, akd, aZ, bS, ah, gb, Jm, fU, dP, ji, uX, IR, cr, qa, adA, cgA, ckY, ei, vt, st, wH, acz, eb, dY, iI, dQ, eN, hm, aAu, LC, aW, aOW, hJ, eM, wB, bWG, nw, qR, aEj, tp, wh, ez, ct, cc, h, jy, dL, vx, Gc, wC, aq, mo, dw, lx, Vy, FV, bm, te, bD, um, bL, eR, dk, uv, sM, cR, C, acj, uT, qp, aT, vH, bK, yb, an, af, ak, qw, ND, aaE, f, bNB, ajl, wg, nM, pZ, mf, lJ, hS, qX, uq, Zl, qb, vr, oH, av, ib, sO, ts, jo, bjP, uQ, eZ, aF, ek, apM, z, uR, aS, tr, tP, us); #c1(NOS1P) c2(NP_057037) c3(nitric oxide synthase-interacting protein) c4(188577) c5(188578, 188579, 188580, 188581, 188582, 188583, 188584, 188585, 188586, 188587, 188588, 188589, 188590, 188591) c6( ) c7(Ou, ya); #c1(NOSTRIN) c2(NP_001034813) c3(nostrin isoform 2) c4(32349) c5(32350, 32351, 32352, 32353, 32354, 32355, 32356) c6( ) c7(gk, al, ac, bN); #c1(NOSTRIN) c2(NP_001165102) c3(nostrin isoform 4) c4(67479) c5(67480, 67481, 67482, 67483, 67484, 67485, 67486, 67487, 67488, 67489) c6( ) c7(gk, al, ac, bN); #c1(NOSTRIN) c2(NP_443178) c3(nostrin isoform 1) c4(229734) c5(229735, 229736, 229737, 229738, 229739) c6( ) c7(gk, al, ac, bN); #c1(NOTCH1) c2(NP_060087) c3(neurogenic locus notch homolog protein 1 preproprotein) c4(194085) c5(194086, 194087, 194088, 194089, 194090, 194091) c6( ) c7(T, os, aC, tQ, yS, if, fd, bB, ci, Qp, aR, ip, aJM, x, aE, dE, pX, K, hu, eL, Zm, cla, up, Jl, aW, Az, uY, g, as, eE, eF, cm, iP, qJ, ou, eM, nB, acw, lq, aep, cj, CD, k, m, lS, ym, iu, wu, aDT, n, qN, b, ft, jz, i, kH, eV, hn, Df, Gc, aeJ, Vt, ack, aq, wU, bfW, dB, hk, eD, Vy, hL, pg, eo, qF, anx, bD, nz, ph, it, B, bL, fM, dA, eR, jp, yk, eX, aA, hy, bzl, V, akG, q, eA, Mg, lg, nv, qK, aT, rO, dz, j, ae, af, ne, jf, afG, bf, s, vT, iU, oV, Y, apk, r, en, cl, bQ, t, iK, amF, ck, sS, baz, nM, iM, dy, im, bt, rA, uG, jh, oZ, Fb, hF, ad, iC, av, hd, fx, ib, Rq, py, ji, wm, nJ, Yi, d, BN, je, qt, ff, eH, cr, eZ, aF, si, iV, iR, bz, arB, ckZ, bE, X, gZ, aS, be, adg, y, CW, jr, BA); #c1(NOTCH1) c2(XP_011517019) c3(neurogenic locus notch homolog protein 1 isoform X1) c4( ) c5(310509) c6(310510) c7(T, os, aC, tQ, yS, if, fd, bB, ci, Qp, aR, ip, aJM, x, aE, dE, pX, K, hu, eL, Zm, cla, up, Jl, aW, Az, uY, g, as, eE, eF, cm, iP, qJ, ou, eM, nB, acw, lq, aep, cj, CD, k, m, lS, ym, iu, wu, aDT, n, qN, b, ft, jz, i, kH, eV, hn, Df, Gc, aeJ, Vt, ack, aq, wU, bfW, dB, hk, eD, Vy, hL, pg, eo, qF, anx, bD, nz, ph, it, B, bL, fM, dA, eR, jp, yk, eX, aA, hy, bzl, V, akG, q, eA, Mg, lg, nv, qK, aT, rO, dz j, ae, af, ne, jf, afG, bf, s, vT, iU, oV, Y, apk, r, en, cl, bQ, t, iK, amF, ck, sS, baz, nM, iM, dy, im, bt, rA, uG, jh, oZ, Fb, hF, ad, iC, av, hd, fx, ib, Rq, py, ji, wm, nJ, Yi, d, BN, je, qt, ff, eH, cr, eZ, aF, si, iV, iR, bz, arB, ckZ, bE, X, gZ, aS, be, adg, y, CW, jr, BA); #c1(NOTCH2) c2(XP_011539821) c3(neurogenic locus notch homolog protein 2 isoform X1) c4( ) c5(338043, 338044, 338045, 338046, 338047, 338048) c6( ) c7(bL, gV, T, fM, bga, wu, if, e, qy, aA, jh, aj, v, qN, oc, Fc, q, J, ai, zn, eA, ip, BI, hd, ib, MS, Rq, av, nv, dE, iu, jz, aT, h, wm, Od, Wr, bA, jf, d, ajT, hG, Jl, aW, cr, r, nY, aF, g, as, hJ, eE, uC, clb, cm, aHY, ck, clc, bLr, bKf, nB, dB, acw, vA, cj, aS, hh, bt, it, B, Ex, hS); #c1(NOTCH2) c2(XP_011539822) c3(neurogenic locus notch homolog protein 2 isoform X2) c4( ) c5(338049, 338050, 338051, 338052, 338053) c6( ) c7(bL, gV, T, fM, bga, wu, if, e, qy, aA, jh, aj, v, qN, oc, Fc, q, J, ai, zn, eA, ip, BI, hd, ib, MS, Rq, av, nv, dE, iu, jz, aT, h, wm, Od, Wr, bA, jf, d, ajT, hG, Jl, aW, cr, r, nY, aF, g, as, hJ, eE, uC, clb, cm, aHY, ck, clc, bLr, bkf, nB, dB, acw, vA, cj, aS, hh, bt, it, B, Ex, hS); #c1(NOTCH3) c2(NP_000426) c3(neurogenic locus notch homolog protein 3 precursor) c4(8847) c5(8848, 8849, 8850, 8851, 8852, 8853, 8854) c6( ) c7(ID, T, ahf, aC, Tb, if, e, al, l, fd, ur, ci, Hr, x, bv, aif, abH, M, K, hm, ly, aW, vs, g, hJ, cm, eM, U, cj, k, m, vl, aw, n, fr, qN, cld, cd, aJr, Mo, clf, kH, hn, Jn, ey, bI, A, MD, ack, agF, adz, lx, hR, te, it, B, cz, cR, v, j, V, eA, PW, Mp, hb, aT, cy, cle, ae, ak, bf, Y, r, en, wA, t, kC, Vq, ck, baz, ac, c, clg, wg, nM, dy, vi, uq, uu, ad, av, Ae, Ot, oj, d, lR, ff, bz, dr, ek, gk, gZ, aS, tr, lC, Ex, BA); #c1(NOTCH3) c2(XP_005259981) c3(neurogenic locus notch homolog protein 3 isoform X1) c4(276352) c5(276353, 276354, 276355, 276356, 276357, 276358, 276359, 276360, 276361, 276362, 276363, 276364, 276365) c6( ) c7(ID, T, ahf, aC, Tb, if, e, al, l, fd, ur, ci, Hr, x, bv, aif, abH, M, K, hm, ly, aW, vs, g, hJ, cm, eM, U, cj, k, m, vl, aw, n, fr, qN, cld, cd, aJr, Mo, clf, kH, hn, Jn, ey, bI, A, MD, ack, agF, adz, lx, hR, te, it, B, cz, cR, v, j, V, eA, PW, Mp, hb, aT, cy, cle, ae, ak, bf, Y, r, en, wA, t, kC, Vq, ck, baz, ac, c, clg, wg, nM, dy, vi, uq, uu, ad, av, Ae, Ot, oj, d, lR, ff, bz, dr, eK, gk, gZ, aS, tr, lC, Ex, BA); #c1(NOTCH4) c2(NP_004548) c3(neurogenic locus notch homolog protein 4 preproprotein) c4(147508, 147509, 147510, 147511, 147512, 147513, 147514) c5(147515, 147516, 147517, 147518, 147519, 147520, 147521, 147522, 147523, 147524, 147525, 147526, 147527, 147528, 147529, 147530, 147531, 147532, 147533, 147534, 147535, 147536, 147537, 147538, 147539, 147540, 147541, 147542, 147543, 147544, 147545, 147546, 147547, 147548, 147549, 147550, 147551, 147552, 147553, 147554, 147555, 147556, 147557, 147558, 147559, 147560, 147561, 147562, 147563, 147564, 147565, 147566, 147567, 147568, 147569, 147570, 147571, 147572, 147573, 147574, 147575, 147576, 147577, 147578, 147579) c6( ) c7(bM, aC, wu, fG, al, jx, mS, ti, uu, ci, C, mF, hF, V, eA, BI, mW, BJ, qK, aT, hD, bK, ey, wm, mY, mV, af, cp, mU, A, B, aW, Y, cr, eZ, abn, pD, cl, aaL, iV, mZ, pb, mX, ck, gk, OW, kP, W, nB, cA, hL, y, mT, BA, vi); #c1(NOTUM) c2(NP_848588) c3 (protein notum homolog precursor) c4(253895, 253896) c5(253897, 253898, 253899, 253900, 253901, 253902, 253903, 253904, 253905, 253906, 253907, 253908) c6( ) c7(B); #c1(NOV) c2(NP_002505) c3(protein NOV homolog precursor) c4(129384) c5(129385, 129386, 129387, 129388, 129389, 129390) c6( ) c7(T, kh, aC, oe, eb, KO, aj, fr, uE, V, eA, ad, J, er, fU, bv, aE, bOL, aT, By, dL, ae, eL, dx, cp, d, iU, aW, Y, eH, iw, as, cl, eF, t, uC, hc, hl, dy, m, Xu, bt); #c1(NOVA1) c2(NP_002506) c3(RNA-binding protein Nova-1 isoform 1) c4(129391, 129392) c5(129393, 129394, 129395, 129396, 129397, 129398, 129399, 129400, 129401, 129402, 129403, 129404) c6( ) c7(akx, clh, oe, e, alv, bK, aen, eA, aR); #c1(NOVA1) c2(NP_006480) c3(RNA-binding protein Nova-1 isoform 2) c4(165583) c5(165584, 165585, 165586, 165587, 165588, 165589) c6( ) c7(akx, clh, oe, e, alv, bk, aen, eA, aR); #c1(NOVA1) c2(NP_006482) c3(RNA-binding protein Nova-1 isoform 3) c4(165590) c5(165591, 165592, 165593, 165594, 165595, 165596) c6( ) c7(akx, clh, oe, e, alv, bK, aen, eA, aR); #c1(NOVA1) c2(XP_011535102) c3(RNA-binding protein Nova-1 isoform X1) c4( ) c5(331892) c6( ) c7(akx, clh, oe, e, alv, bK, aen, eA, aR); #c1(NOVA2) c2(NP_002507) c3(RNA-binding protein Nova-2) c4(129405) c5(129406, 129407, 129408, 129409, 129410, 129411) c6( ) c7(bM, T, Uc, clh, aZ, aD, if, bO, aA, Bm, cz, clj, bS, j, ct, ci, v, qN, fr, Ul, q, aR, tO, eA, x, hd, aOu, oz, aT, K, lQ, he, aDp, eN, af, Jc, OV, d, s, aW, aFt, r, fe, aF, g, pm, Z, FX, t, f, cm, qa, op, hq, ck, bN, pH, ac, cli, nB, vA, ayS, qy, at, Rm, aS, m, k, dy, xg); #c1(NOX1) c2(NP_001258744) c3 (NADPH oxidase 1 isoform 3) c4(96470) c5(96471, 96472, 96473, 96474, 96475, 96476, 96477, 96478, 96479, 96480, 96481, 96482, 96483, 96484, 96485, 96486, 96487) c6( ) c7(bL, T, dD, OI, al, cz, bB, eq, ci, C, cd, q, ad, iu, BI, aT, h, ji, K, ae, cp, wC, YL, d, lR, s, CH, Cu, Y, Rr, r, kt, g, uC, ck, D, bE, eB, aS, vt, X); #c1(NOX1) c2(NP_008983) c3(NADPH oxidase 1 isoform 1) c4(170047, 170048, 170049) c5(170050, 170051, 170052, 170053, 170054, 170055, 170056) c6( ) c7(bL, T, dD, OI, al, cz, bB, eq, ci, C, cd, q, ad, iu, BI, aT, h, ji, K, ae, cp, wC, YL, d, lR, s, cH, Cu, Y, Rr, r, kt, g, uC, ck, D, bE, eB, aS, vt, X); #c1(NOX1) c2(NP_039249) c3(NADPH oxidase 1 isoform 2) c4(176932, 176933) c5(176934, 176935, 176936, 176937, 176938, 176939, 176940, 176941, 176942, 176943, 176944, 176945, 176946, 176947, 176948, 176949, 176950) c6( ) c7(bL, T, dD, OI, al, cz, bB, eq, ci, C, cd, q, ad, iu, Bl, aT, h, ji, K, ae, cp, wC, YL, d, lR, s, cH, Cu, Y, Rr, r, kt, g, uC, ck, D, bE, eB, aS, vt, X); #c1(NOX3) c2(NP_056533) c3(NADPH oxidase 3) c4(187978) c5(187979, 187980, 187981, 187982, 187983, 187984, 187985, 187986) c6( ) c7(bM, aC, ml, cz, h, fe, k); #c1(NOX4) c2(NP_001137308) c3(NADPH oxidase 4 isoform b) c4(55431) c5(55432, 55433, 55434, 55435, 55436, 55437, 55438, 55439, 55440, 55441, 55442, 55443) c6( ) c7(bL, bM, T, lD, aC, eR, fG, e, al, jx, cz, cR, bB, aj, dG, j, bh, fr, cd, J, eA, BY, eJ, hd, aE, sN, aT, h, lG, cy, K, bq, cp, bA, wC, Lu, A, d, B, bf, cW, aW, Y, ev, aq, kp, as, iR, t, iK, bz, LB, ck, es, ac, c, bE, go, aS, lC, X); #c1(NOX4) c2(NP_001278856) c3(NADPH oxidase 4 isoform e) c4(110726) c5(110727, 110728, 110729, 110730, 110731, 110732, 110733, 110734, 110735, 110736) c6( ) c7(bL, bM, T, lD, aC, eR, fG, e, al, jx, cz, cR, bB, aj, dG, j, bh, fr, cd, J, eA, BY, eJ, hd, aE, sN, aT, h, lG, cy, K, bq, cp, bA, wC, Lu, A, d, B, bf, cW, aW, Y, ev, aq, kp, as, iR, t, iK, bz, LB, ck, es, ac, c, bE, go, aS, lC, X); #c1(NOX4) c2(NP_001278858) c3(NADPH oxidase 4 isoform f) c4(110737) c5( ) c6( ) c7(bL, bM, T, lD, aC, eR, fG, e, al, jx, cz, cR, bB, aj, dG, j, bh, fr, cd, J, eA, BY, eJ, hd, aE, sN, aT, h, IG, cy, K, bq, cp, bA, wC, Lu, A, d, B, bf, cW, aW, Y, ev, aq, kp, as, iR, t, iK, bz, LB, ck, es, ac, c, bE, go, aS, lC, X); #c1(NOX4) c2(NP_058627) c3(NADPH oxidase 4 isoform a) c4(192840, 192841) c5(192842, 192843, 192844, 192845, 192846, 192847, 192848) c6( ) c7(bL, bM, T, lD, aC, eR, fG, e, al, jx, cz, cR, bB, aj, dG, j, bh, fr, cd, J, eA, BY, eJ, hd, aE, sN, aT, h, lG, cy, K, bq, cp, bA, wC, Lu, A, d, B, bf, cW, aW, Y, ev, aq, kp, as, iR, t, iK, bz, LB, ck, es, ac, c, bE, go, aS, lC, X); #c1(NOX4) c2(XP_011541159) c3(NADPH oxidase 4 isoform X1) c4(339717) c5(339718, 339719, 339720, 339721, 339722, 339723, 339724, 339725, 339726, 339727, 339728, 339729, 339730, 339731, 339732, 339733, 339734) c6( ) c7(bL, bM, T, lD, aC, eR, fG, e, al, jx, cz, cR, bB, aj, dG, j, bh, fr, cd, J, eA, BY, eJ, hd, aE, sN, aT, h, lG, cy, K, bq, cp, bA, wC, Lu, A, d, B, bf, cW, aW, Y, ev, aq, kp, as, iR, t, iK, bz, LB, ck, es, ac, c, bE, go, aS, lC, X); #c1(NOX4) c2(XP_011541160) c3(NADPH oxidase 4 isoform X3) c4(339735) c5(339736, 339737, 339738, 339739, 339740, 339741) c6( ) c7(bL, bM, T, lD, aC, eR, fG, e, al, jx, cz, cR, bB, aj, dG, j, bh, fr, cd, J, eA, BY, eJ, hd, aE, sN, aT, h, lG, cy, K, bq, cp, bA, wC, Lu, A, d, B, bf, cW, aW, Y, ev, aq, kp, as, iR, t, iK, bz, LB, ck, es, ac, c, bE, go, aS, lC, X); #c1(NOX4) c2(XP_011541161) c3(NADPH oxidase 4 isoform X2) c4( ) c5(339742, 339743, 339744, 339745, 339746, 339747) c6( ) c7(bL, bM, T, lD, aC, eR, fG, e, al, jx, cz, cR, bB, aj, dG, j, bh, fr, cd, J, eA, BY, eJ, hd, aE, sN, aT, h, lG, cy, K, bq, cp, bA, wC, Lu, A, d, B, bf, cW, aW, Y, ev, aq, kp, as, iR, t, iK, bz, LB, ck, es, ac, c, bE, go, aS, lC, X); #c1(NOX5) c2(NP_001171708) c3(NADPH oxidase 5 isoform 2) c4(71361) c5(71362, 71363, 71364, 71365, 71366, 71367) c6( ) c7(T, dD, pf, ea, acl, aw, cz, aj, j, eq, aY, cd, ad, J, h, K, ae, hm, d, g, bz, eB, lI, fP, gZ, dy, cE); #c1(NOX5) c2(NP_001171709) c3(NADPH oxidase 5 isoform 3) c4(71368) c5(71369, 71370, 71371, 71372, 71373, 71374) c6( ) c7(T, dD, pf, ea, acl, aw, cz, aj, j, eq, aY, cd, ad, J, h, K, ae, hm, d, g, bz, eB, lI, fP, gZ, dy, cE); #c1(NOX5) c2(NP_078781) c3(NADPH oxidase 5 isoform 1) c4(215448) c5(215449, 215450, 215451, 215452, 215453, 215454) c6( ) c7(T, dD, pf, ea, acl, aw, cz, aj, j, eq, aY, cd, ad, J, h, K, ae, hm, d, g, bz, eB, lI, fP, gZ, dy, cE); #c1(NOXA1) c2(NP_001242997) c3(NADPH oxidase activator 1 isoform 3) c4(89506) c5(89507, 89508, 89509, 89510, 89511, 89512) c6( ) c7(T, dD, bE, ea, bB, aS, K, eq, g); #c1(NOXA1) c2(NP_006638) c3(NADPH oxidase activator 1 isoform 1) c4(166827) c5(166828, 166829, 166830, 166831, 166832, 166833) c6( ) c7(T, dD, bE, ea, bB, aS, K, eq, g); #c1(NOX01) c2(NP_653204) c3(NADPH oxidase organizer 1 isoform a) c4(239057) c5(239058, 239059, 239060, 239061, 239062, 239063) c6( ) c7(YL, aA, bvS, K, g, ZN); #c1(NOX01) c2(NP_751907) c3(NADPH oxidase organizer 1 isoform b) c4(248508) c5(248509, 248510, 248511, 248512, 248513, 248514) c6( ) c7(YL, aA, bvS, K, g, ZN); #c1(NOX01) c2(XP_005255156) c3(NADPH oxidase organizer 1 isoform X1) c4(273563) c5(273564, 273565, 273566, 273567, 273568, 273569) c6( ) c7(YL, aA, bvS, K, g, ZN); #c1(NPAP1) c2(NP_061831) c3(nuclear pore-associated protein 1) c4(200673) c5(200674, 200675, 200676, 200677, 200678, 200679, 200680) c6( ) c7(Y, rc, agl); #c1(NPAS2) c2(NP_002509) c3(neuronal PAS domain-containing protein 2) c4(129412) c5(129413, 129414, 129415, 129416, 129417) c6( ) c7(aPY, T, d, iH, aW, iW, jh, aT, ta, bk, ei, Z, oi, ae, ad, bN, dS); #c1(NPAS2) c2(XP_005264010) c3(neuronal PAS domain-containing protein 2 isoform X1) c4(278644) c5(278645, 278646, 278647, 278648, 278649, 278650, 278651) c6( ) c7(aPY, T, d, iH, aW, iW, jh, aT, ta, bK, ei, Z, oi, ae, ad, bN, dS); #c1(NPAS3) c2(NP_001158221) c3(neuronal PAS domain-containing protein 3 isoform 1) c4(63436, 63437) c5(63438, 63439, 63440, 63441, 63442, 63443, 63444, 63445, 63446, 63447, 63448, 63449) c6( ) c7(d, bO, gl, uJ, bK, nA, as, he, ZW, eA, xt, jb); #c1(NPAS3) c2(NP_001159365) c3(neuronal PAS domain-containing protein 3 isoform 4) c4(63869) c5(63870, 63871, 63872, 63873, 63874, 63875) c6( ) c7(d, bO, gl, uJ, bK, nA, as, he, ZW, eA, xt, jb); #c1(NPAS3) c2(NP_071406) c3(neuronal PAS domain-containing protein 3 isoform 2) c4(211473) c5(211474, 211475, 211476, 211477, 211478, 211479, 211480, 211481, 211482, 211483, 211484, 211485) c6( ) c7(d, bO, gl, uJ, bK, nA, as, he, ZW, eA, xt, jb); #c1(NPAS3) c2(NP_775182) c3(neuronal PAS domain-containing protein 3 isoform 3) c4(249650) c5(249651, 249652, 249653, 249654, 249655, 249656) c6( ) c7(d, bO, gl, uJ, bK, nA, as, he, ZW, eA, xt, jb); #c1(NPAS3) c2(XP_005268049) c3(neuronal PAS domain-containing protein 3 isoform X4) c4(281426) c5( ) c6( ) c7(d, bO, gl, uJ, bK, nA, as, he, ZW, eA, xt, jb); #c1(NPAS4) c2(NP_849195) c3(neuronal PAS domain-containing protein 4) c4(254500, 254501) c5(254502, 254503, 254504, 254505, 254506, 254507, 254508, 254509, 254510, 254511, 254512) c6( ) c7(cm, Hx); #c1(NPAS4) c2(XP_011543240) c3(neuronal PAS domain-containing protein 4 isoform X1) c4( ) c5(342027) c6( ) c7(cm, Hx); #c1(NPAT) c2(NP_002510) c3(protein NPAT) c4(129418) c5(129419, 129420, 129421, 129422, 129423, 129424) c6( ) c7(if, eX, VT, cj, eV, dL, bC, qN); #c1(NPB) c2(NP_683694) c3(neuropeptide B preproprotein) c4(242386) c5(242387, 242388, 242389, 242390, 242391, 242392, 242393) c6(242394) c7(kl, clk, ek, cG); #c1(NPBWR1) c2(NP_005276) c3(neuropeptides B/W receptor type 1) c4(154649) c5(154650, 154651, 154652, 154653, 154654, 154655) c6( ) c7(c, n, aj, fu, ae, ad); #c1(NPC1) c2(NP_000262) c3(Niemann-Pick C1 protein precursor) c4(5895, 5896) c5(5897, 5898, 5899, 5900, 5901, 5902, 5903, 5904, 5905, 5906, 5907, 5908, 5909, 5910, 5911, 5912, 5913, 5914, 5915) c6( ) c7(oO, bL, T, dD, iW, e, aOE, al, aA, cz, fd, v, j, ci, qf, aVK, ee, eq, q, aR, bCZ, eA, ke, Hx, MR, WY, rC, oW, h, K, lQ, qM, fb, af, avV, d, clm, clk, xs, hl, fc, r, lo, aF, g, anW, bz, VZ, AH, U, cU, Lp, akH, Lq, act, cP, du, Al, ek); #c1(NPC1) c2(XP_005258335) c3(Niemann-Pick C1 protein isoform X2) c4(275640) c5( ) c6( ) c7(oO, bL, T, dD, iW, e, aOE, al, aA, cz, fd, v, j, ci, qf, aVK, ee, eq, q, aR, bCZ, eA, ke, Hx, MR, WY, rC, oW, h, K, lQ, qM, fb, af, avV, d, clm, clk, xs, hl, fc, r, lo, aF, g, anW, bz, VZ, AH, U, cU, Lp, akH, Lq, act, cP, du, Al, ek); #c1(NPC1L1) c2(NP_001095118) c3(Niemann-Pick C1-like protein 1 isoform 2 precursor) c4(40787, 40788) c5(40789, 40790, 40791, 40792, 40793, 40794, 40795, 40796, 40797, 40798, 40799, 40800) c6( ) c7(jF, cl, ZV, cH, gZ, aj, v, j, bq, dQ, ZG); #c1(NPC1L1) c2(NP_037521) c3(Niemann-Pick C1-like protein 1 isoform 1 precursor) c4(175984) c5(175985, 175986, 175987, 175988, 175989, 175990) c6( ) c7(jF, cl, ZV, cH, gZ, aj, v, j, bq, dQ, ZG); #c1(NPC1L1) c2(XP_011513629) c3(Niemann-Pick C1-like protein 1 isoform X2) c4( ) c5(305942) c6( ) c7(jF, cl, ZV, cH, gZ, aj, v, j, bq, dL, ZG); #c1(NPC2) c2(NP_006423) c3(epididymal secretory protein E1 precursor) c4(164920, 164921) c5(164922, 164923, 164924, 164925, 164926, 164927, 164928, 164929, 164930, 164931, 164932, 164933, 164934) c6( ) c7(bL, T, iW, al, fr, bX, hd, Db, WY, aT, jy, af, Y, aXY, dq, aq, iR, dr, gk, nM, aDA, nB, U, cU, Lq, act, du, ek); #c1(NPDC1) c2(NP_056207) c3(neural proliferation differentiation and control protein 1 precursor) c4(186318, 186319, 186320) c5(186321, 186322, 186323, 186324, 186325, 186326, 186327, 186328, 186329, 186330, 186331, 186332) c6( ) c7(aT); #c1(NPEPL1) c2(NP_001191801) c3(probable aminopeptidase NPEPL1 isoform 2) c4(81820) c5(81821, 81822, 81823, 81824, 81825, 81826) c6( ) c7(aW, aT); #c1(NPEPL1) c2(NP_001191802) c3(probable aminopeptidase NPEPL1 isoform 3) c4(81827) c5(81828, 81829, 81830, 81831, 81832, 81833) c6( ) c7(aW, aT); #c1(NPEPL1) c2(NP_078939) c3(probable aminopeptidase NPEPL1 isoform 1) c4(216195) c5(216196, 216197, 216198, 216199, 216200, 216201) c6( ) c7(aW, aT); #c1(NPEPPS) c2(NP_006301) c3(puromycin-sensitive aminopeptidase) c4(163829, 163830, 163831, 163832, 163833) c5( ) c6( ) c7(gh, T, dA, WA, rQ, Hn, n, Fb, ci, eW, ad, aE, aT, ji, ae, Ub, aW, cr, g, t, hq, pu, GU, Jr, m, LS); #c1(NPEPPS) c2(XP_011523798) c3(puromycin-sensitive aminopeptidase isoform X1) c4( ) c5(318298, 318299, 318300, 318301, 318302, 318303, 318304, 318305, 318306, 318307, 318308, 318309, 318310, 318311, 318312, 318313, 318314, 318315, 318316, 318317, 318318, 318319, 318320, 318321, 318322, 318323, 318324, 318325, 318326, 318327) c6( ) c7(gh, T, dA, WA, rQ, Hn, n, Fb, ci, eW, ad, aE, aT, ji, ae, Ub, aW, cr, g, t, hq, pu, GU, Jr, m, LS); #c1(NPFF) c2(NP_003708) c3(pro-FMRFamide-related neuropeptide FF preproprotein) c4(140326) c5(140327, 140328, 140329, 140330, 140331, 140332, 140333, 140334) c6(140335, 140336) c7(cm, gu); #c1(NPFFR2) c2(NP_00138228) c3(neuropeptide FF receptor 2 isoform 3) c4(56062) c5(56063, 56064, 56065, 56066, 56067, 56068, 56069) c6( ) c7(cm, v, j); #c1(NPFFR2) c2(NP_004876) c3(neuropeptide FF receptor 2 isoform 1) c4(150474, 150475, 150476) c5(150477, 150478, 150479, 150480, 150481, 150482, 150483, 150484, 150485, 150486, 150487) c6( ) c7(cm, v, j); #c1(NPFFR2) c2(NP_444264) c3(neuropeptide FF receptor 2 isoform 2) c4(230201) c5(230202, 230203, 230204, 230205, 230206, 230207) c6( ) c7(cm, v, j); #c1(NPHP1) c2(NP_000263) c3(nephrocystin-1 isoform 1) c4(5916) c5(5917, 5918, 5919, 5920, 5921, 5922) c6( ) c7(aKZ, blk, W, eR, vj, wB, KA, vw, vm, Sq, nw, dy, nA, uD, GF, Sp, Tp, vN, aY); #c1(NPHP1) c2(NP_001121650) c3(nephrocystin-1 isoform 3) c4(46020) c5(46021, 46022, 46023, 46024, 46025, 46026) c6( ) c7(aKZ, blk, W, eR, vj, wB, KA, vw, vm, Sq, nw, dy, nA, uD, GF, Sp, Tp, vN, aY); #c1(NPHP1) c2(NP_001121651) c3(nephrocystin-1 isoform 4) c4(46027) c5(46028, 46029, 46030, 46031, 46032, 46033) c6( ) c7(aKZ, blk, W, eR, vj, wB, KA, vw, vm, Sq, nw, dy, nA, uD, GF, Sp, Tp, vN, aY); #c1(NPHP1) c2(NP_997064) c3(nephrocystin-1 isoform 2) c4(265012) c5(265013, 265014, 265015, 265016, 265017, 265018) c6( ) c7(aKZ, blk, W, eR, vj, wB, KA, vw, vm, Sq, nw, dy, nA, uD, GF, Sp, Tp, vN, aY); #c1(NPHP3) c2(NP_694972) c3(nephrocystin-3) c4(245513, 245514, 245515, 245516, 245517, 245518) c5(245519, 245520, 245521, 245522, 245523, 245524, 245525, 245526, 245527, 245528, 245529, 245530, 245531, 245532, 245533, 245534, 245535, 245536, 245537, 245538, 245539, 245540, 245541, 245542, 245543, 245544, 245545, 245546, 245547, 245548, 245549, 245550, 245551, 245552, 245553, 245554) c6( ) c7(aKZ, eR, BV, cln, bKt, wB, clo, vm, nw, v, Nj, aBZ, bTa, Sp, nC, vN, aY); #c1(NPHP4) c2(NP_001278522) c3(nephrocystin-4 isoform b) c4( ) c5(109788, 109789, 109790, 109791, 109792) c6( ) c7(akZ, clq, clp, vm, clr, dy, bD, Sp, nC, Nh); #c1(NPHP4) c2(XP_011539522) c3(nephrocystin-4 isoform X6) c4(337722) c5(337723) c6( ) c7(aKZ, clq, clp, vm, clr, dy, bD, Sp, nC, Nh); #c1(NPHS1) c2(NP_004637) c3(nephrin precursor) c4(148410, 148411) c5(148412, 148413, 148414, 148415, 148416, 148417, 148418, 148419, 148420, 148421, 148422, 148423) c6( ) c7(bL, Kl, eR, dk, cz, zN, n, dG, ah, aY, er, cls, vw, ey, K, Bz, wt, sj, A, PK, vj, gi, aEW, uC, dr, sk, vW, wE, hc, va, dy, fy, ul, jr); #c1(NPHS2) c2(NP_001284504) c3(podocin isoform 2) c4(111937) c5(111938, 111939, 111940, 111941, 111942, 111943) c6( ) c7(dD, eR, ea, dk, cz, zN, n, cQ, ah, aj, uE, j, eq, aY, uW, UI, J, er, fU, vw, vH, h, ey, wt, DX, af J, sj, A, PK, acF, gi, aEW, agn, dr, sk, aLO, vW, gZ, va, dy, R, oQ, ul); #c1(NPHS2) c2(NP_055440) c3(podocin isoform 1) c4(181151) c5(181152, 181153, 181154, 181155, 181156, 181157) c6( ) c7(dD, eR, ea, dk, cz, zN, n, cQ, ah, aj, uE, j, eq, aY, uW, Ul, J, er, fU, vw, vH, h, ey, wt, DX, afJ, sj, A, PK, acF, gi, aEW, agn, dr, sk, aLO, vW, gZ, va, dy, R, oQ, ul); #c1(NPL) c2(NP_001186979) c3(N-acetylneuraminate lyase isoform 2) c4(78915, 78916, 78917) c5(78918, 78919, 78920, 78921, 78922, 78923, 78924, 78925, 78926, 78927, 78928, 78929, 78930, 78931, 78932, 78933, 78934, 78935) c6( ) c7(T, cz, mS, ww, q, ad, mW, iX, bK, ey, mY, mV, ae, ml, A, tb, sp, r, fe, gl, SG, iV, eE, mZ, mX, arz, Kr, uJ, mT); #c1(NPL) c2(NP_001186980) c3(N-acetylneuraminate lyase isoform 4) c4(78936) c5(78937, 78938, 78939, 78940, 78941, 78942, 78943, 78944, 78945, 78946) c6( ) c7(T, cz, mS, ww, q, ad, mW, iX, bk, ey, mY, mV, ae, mU, A, tb, sp, r, fe, gl, SG, iV, eE, mZ, mX, arz, Kr, uJ, mT); #c1(NPL) c2(NP_001186981) c3(N-acetylneuraminate lyase isoform 5) c4( ) c5(78947, 78948, 78949, 78950, 78951, 78952) c6( ) c7(T, cz, mS, ww, q, ad, mW, iX, bK, ey, mY, mV, ae, mU, A, tb, sp, r, fe, gl, SG, iV, eE, mZ, mX, arz, Kr, uJ, mT); #c1(NPL) c2(NP_110396) c3(N-acetylneuraminate lyase isoform 1) c4(220033, 220034) c5(220035, 220036, 220037, 220038, 220039, 220040, 220041, 220042, 220043, 220044) c6( ) c7(T, cz, mS, ww, q, ad, mW, iX, bK, ey, mY, mV, ae, mU, A, tb, sp, r, fe, gl, SG, iV, eE, mZ, mX, arz, Kr, uJ, mT); #c1(NPLOC4) c2(NP_060391) c3(nuclear protein localization protein 4 homolog) c4(195791) c5(195792, 195793, 195794, 195795, 195796, 195797) c6( ) c7(t); #c1(NPLOC4) c2(XP_011523281) c3(nuclear protein localization protein 4 homolog isoform X1) c4(317791, 317792) c5(317793, 317794, 317795, 317796, 317797, 317798, 317799, 317800, 317801, 317802, 317803, 317804) c6( ) c7(t); #c1(NPM1) c2(NP_001032827) c3(nucleophosmin isoform 3) c4(31476) c5(31477, 31478, 31479, 31480, 31481, 31482) c6( ) c7(T, os, al, fd, bB, ci, x, aE, lv, K, eL, aW, CT, bFG, Fu, eF, cm, bw, lt, eM, iT, dc, cj, k, Wx, n, fr, TI, br, b, KN, h, eV, dL, PX, aq, yq, cM, oU, bP, hk, hL, pg, B, fM, dA, eX, aA, ol, q, er, dn, aT, dz, adV, jj, s, iL, xR, oV, iZ, bvJ, r, gm, en, cl, ck, Ta, Uq, bt, iW, jh, bs, oZ, hF, ad, av, fX, ds, bvF, d, et, ff, aF, iR, bz, bE, i, KS, bEt, Tc); #c1(NPM1) c2(NP_002511) c3 (nucleophosmin isoform 1) c4(129425) c5(129426, 129427, 129428, 129429, 129430, 129431, 129432, 129433, 129434, 129435) c6( ) c7(T, os, al, fd, bB, ci, x, aE, lv, K, eL, aW, CT, bFG, Fu, eF, cm, bw, Qt, eM, iT, dc, cj, k, Wx, n, fr, TI, br, b, KN, h, eV, dL, PX, aq, yq, cM, oU, bP, hk, hL, pg, B, fM, dA, eX, aA, ol, q, er, dn, aT, dz, adV, jj, s, iU, xR, oV, iZ, bvJ, r, gm, en, cl, ck, Ta, Uq, bt, iW, jh, bs, oZ, hF, ad, av, fX, ds, bvF, d, et, ff, aF, iR, bz, bE, i, kS, bEt, Tc); #c1(NPM1) c2(NP_954654) c3(nucleophosmin isoform 2) c4(261176) c5(261177, 261178, 261179, 261180, 261181, 261182) c6( ) c7(T, os, al, fd, bB, ci, x, aE, lv, K, eL, aW, CT, bFG, Fu, eF, cm, bw, Qt, eM, iT, dc, cj, k, Wx, n, fr, TI, br, b, KN, h, eV, dL, PX, aq, yq, cM, oU, bP, hk, hL, pg, B, fM, dA, eX, aA, ol, q, er, dn, aT, dz, adV, jj, s, iU, xR, oV, iZ, bvJ, r, gm, en, cl, ck, Ta, Uq, bt, iW, jh, bs, oZ, hF, ad, av, fx, ds, bvF, d, et, ff, aF, iR, bz, bE, i, kS, bEt, Tc); #c1(NPM2) c2(NP_001273610) c3(nucleoplasmin-2 isoform 2) c4(105435) c5(105436, 105437, 105438, 105439, 105440, 105441) c6( ) c7(ck, bt, aT); #c1(NPM2) c2(NP_877724) c3(nucleoplasmin-2 isoform 1) c4(256934) c5(256935, 256936, 256937, 256938, 256939, 256940, 256941, 256942, 256943, 256944, 256945, 256946, 256947, 256948) c6( ) c7(ck, bt, aT); #c1(NPM2) c2(XP_011542661) c3(nucleoplasmin-2 isoform X2) c4( ) c5(341222, 341223, 341224, 341225) c6( ) c7(ck, bt, aT); #c1(NPNT) c2(NP_001028219) c3(nephronectin isoform B precursor) c4(30367) c5(30368, 30369, 30370, 30371, 30372, 30373, 30374, 30375) c6(30376, 30377) c7(aW, Y, aT, at, Dp, aa); #c1(NPNT) c2(NP_001171619) c3(nephronectin isoform A precursor) c4(70934) c5(70935, 70936, 70937, 70938, 70939, 70940, 70941, 70942) c6(70943, 70944) c7(aW, Y, aT, at, Dp, aa); #c1(NPNT) c2(NP_001171620) c3(nephronectin isoform C precursor) c4(70945) c5(70946, 70947, 70948, 70949, 70950, 70951, 70952, 70953) c6(70954, 70955) c7(aW, Y, aT, at, Dp, aa); #c1(NPNT) c2(NP_001171621) c3(nephronectin isoform D precursor) c4(70956, 70957) c5(70958, 70959, 70960, 70961, 70962, 70963, 70964, 70965, 70966, 70967, 70968, 70969, 70970, 70971, 70972, 70973) c6(70974, 70975, 70976, 70977) c7(aW, Y, aT, at, Dp, aa); #c1(NPNT) c2(NP_001171622) c3(nephronectin isoform E precursor) c4(70978) c5(70979, 70980, 70981, 70982, 70983, 70984, 70985, 70986) c6(70987, 70988) c7(aW, Y, al, at, Dp, aa); #c1(NPPC) c2(NP_077720) c3(C-type natriuretic peptide precursor) c4(215274) c5(215275, 215276, 215277, 215278, 215279, 215280, 215281, 215282, 215283, 215284, 215285, 215286, 215287, 215288) c6(215289, 215290, 215291, 215292) c7(bM, dD, ea, e, aw, cz, aNN, cR, tc, bS, ah, ct, bBb, j, eq, GC, Fc, cd, ad, aRo, ws, Bc, rD, hP, K, he, af, yo, si, as, Z, kT, gZ, dy, xb, vQ); #c1(NPR1) c2(NP_000897) c3(atrial natriuretic peptide receptor 1 precursor) c4(17005) c5(17006, 17007, 17008, 17009, 17010, 17011) c6( ) c7(bL, sY, T, e, aw, gN, cz, vr, v, j, cd, ad, h, ae, Y, ul, t, uC, qa, gZ, dy, m, fy, it); #c1(NPR1) c2(XP_005245275) c3(atrial natriuretic peptide receptor 1 isoform X3) c4(266381) c5(266382, 266383, 266384, 266385, 266386, 266387, 266388, 266389, 266390, 266391, 266392, 266393) c6( ) c7(bL, sY, T, e, aw, gN, cz, vr, v, j, cd, ad, h, ae, Y, ul, t, uC, qa, gZ, dy, m, fy, it); #c1(NPR1) c2(XP_006711405) c3(atrial natriuretic peptide receptor 1 isoform X1) c4(286007) c5( ) c6( ) c7(bL, sY, T, e, aw, gN, cz, vr, v, j, cd, ad, h, ae, Y, ul, t, uC, qa, gZ, dy, m, fy, it); #c1(NPR2) c2(NP_003986) c3(atrial natriuretic peptide receptor 2 precursor) c4(142655) c5(142656, 142657, 142658, 142659, 142660, 142661) c6( ) c7(bL, T, yo, ac, s, e, cz, rD, ade, bBb, mo, pk, rv, cd, qa, ek, bBg); #c1(NPR3) c2(NP_000899) c3(atrial natriuretic peptide receptor 3 isoform 2 precursor) c4(17012) c5(17013, 17014, 17015, 17016, 17017, 17018) c6( ) c7(qa, bM, jf, tW, ac, Bc, e, M, cz, vy, v, j, ji, he, cd, ad); #c1(NPR3) c2(NP_001191304) c3(atrial natriuretic peptide receptor 3 isoform 1 precursor) c4(81351) c5(81352, 81353, 81354, 81355, 81356, 81357) c6( ) c7(qa, bM, jf, tW, ac, Bc, e, M, cz, vy, v, j, ji, he, cd, ad); #c1(NPR3) c2(NP_001191305) c3(atrial natriuretic peptide receptor 3 isoform 3) c4(81358) c5(81359, 81360, 81361, 81362, 81363, 81364) c6( ) c7(qa, bM, jf, tW, ac, Bc, e, M, cz, vy, v, j, ji, he, cd, ad); #c1(NPRL2) c2(NP_006536) c3(nitrogen permease regulator 2-like protein) c4(166082) c5(166083, 166084, 166085, 166086, 166087, 166088) c6( ) c7(av, yo, s, eM, ff, rD, bBb, mo, en, qN, g, bz, cd, bBg); #c1(NPRL3) c2(NP_001034565) c3(nitrogen permease regulator 3-like protein isoform 2) c4( ) c5(31798, 31799, 31800, 31801, 31802) c6( ) c7(jf, BU, ER); #c1(NPRL3) c2(NP_001070818) c3(nitrogen permease regulator 3-like protein isoform 1) c4(34842, 34843) c5(34844, 34845, 34846, 34847, 34848, 34849) c6( ) c7(jf, BU, ER); #c1(NPRL3) c2(NP_001230 (76) c3(nitrogen permease regulator 3-like protein isoform 3) c4( ) c5(86053) c6( ) c7(jf, BU, ER); #c1(NPRL3) c2(NP_001230 (78) c3(nitrogen permease regulator 3-like protein isoform 4) c4(86054, 86055) c5(86056, 86057, 86058, 86059, 86060, 86061, 86062, 86063, 86064, 86065, 86066) c6( ) c7(jf, BU, ER); #c1(NPRL3) c2(XP_011520973) c3(nitrogen permease regulator 3-like protein isoform X6) c4( ) c5(315950) c6( ) c7(jf, BU, ER); #c1(NPS) c2(NP_001025184) c3(neuropeptide S precursor) c4(29464) c5(29465, 29466, 29467, 29468, 29469, 29470) c6( ) c7(ID, bM, T, hq, pn, wM, tb, eM, ju, cu, abj, ww, K, lC, BY); #c1(NPSR1) c2(NP_001287863) c3(neuropeptide S receptor isoform F) c4(113062, 113063) c5(113064, 113065, 113066, 113067, 113068, 113069, 113070, 113071, 113072, 113073, 113074, 113075, 113076, 113077, 113078, 113079, 113080, 113081) c6( ) c7(bM, Dw, aCA, fF, ww, aql, pM, clt, baA, ju, alO, Ds, abj, K, aa, tb, sp, zS, mQ, cr, fe, iV, eE, Jh, hq, cT, z, Jp, JM, uJ, y, qc); #c1(NPSR1) c2(NP_001287864) c3(neuropeptide S receptor isoform G) c4(113082) c5(113083, 113084, 113085, 113086, 113087, 113088, 113089, 113090, 113091, 113092, 113093, 113094) c6( ) c7(bM, Dw, aCA, fF, ww, aql, pM, clt, baA, ju, alO, Ds, abj, K, aa, tb, sp, zS, md, cr, fe, iV, eE, Jh, hq, cT, z, Jp, JM, uJ, y, qc); #c1(NPSR1) c2(NP_997055) c3 (neuropeptide S receptor isoform A) c4(264982, 264983, 264984, 264985) c5(264986, 264987, 264988, 264989, 264990, 264991, 264992, 264993, 264994, 264995, 264996, 264997) c6( ) c7(bM, Dw, aCA, fF, ww, aql, pM, clt, baA, ju, alO, Ds, abj, K, aa, tb, sp, zS, mQ, cr, fe, iV, eE, Jh, hq, cT, z, Jp, JM, uJ, y, qc); #c1(NPSR1) c2(NP_997056) c3(neuropeptide S receptor isoform B) c4(264998) c5(264999, 265000, 265001, 265002, 265003, 265004) c6( ) c7(bM, Dw, aCA, fF, ww, aql, pM, clt, baA, ju, alO, Ds, abj, K, aa, tb, sp, zS, mQ, cr, fe, iV, eE, Jh, hq, cT, z, Jp, JM, uJ, y, qc); #c1(NPTN) c2(NP_001154835) c3(neuroplastin isoform c precursor) c4(61177, 61178) c5(61179, 61180, 61181, 61182, 61183, 61184) c6( ) c7(bnN, oT, GU, aW, cz, aT, j); #c1(NPTN) c2(NP_036560) c3(neuroplastin isoform b precursor) c4(174401, 174402) c5(174403, 174404, 174405, 174406, 174407, 174408) c6( ) c7(bnN, oT, GU, aW, cz, aT, j); #c1(NPTN) c2(NP_059429) c3(neuroplastin isoform a precursor) c4(193288) c5(193289, 193290, 193291, 193292, 193293, 193294, 193295, 193296, 193297, 193298, 193299, 193300) c6( ) c7(bnN, oT, GU, aW, cz, aT, j); #c1(NPTN) c2(XP_011519754) c3(neuroplastin isoform X1) c4( ) c5(314443, 314444, 314445, 314446, 314447, 314448) c6( ) c7(bnN, oT, GU, aW, cz, aT, j); #c1(NPTX1) c2(NP_002513) c3(neuronal pentraxin-1 precursor) c4(129436, 129437) c5(129438, 129439, 129440, 129441, 129442, 129443, 129444, 129445, 129446, 129447, 129448, 129449) c6( ) c7(if, ft, qN, r); #c1(NPTX2) c2(NP_002514) c3(neuronal pentraxin-2 precursor) c4(129450) c5(129451, 129452, 129453, 129454, 129455, 129456) c6( ) c7(bgL, T, aC, nx, bS, ci, eA, no, aE, bgN, vc, an, awL, ajU, d, adF, nY, kp, kC, nC, bN, ajS, mx, eQ, clu); #c1(NPTXR) c2(NP_055108) c3(neuronal pentraxin receptor) c4(178988) c5(178989, 178990, 178991, 178992, 178993, 178994, (78995) c6( ) c7(T, ft, cd, eu, mo); #c1(NPVF) c2(NP_071433) c3(pro-FMRFamide-related neuropeptide VF precursor) c4(211637) c5(211638, 211639, 211640, 211641, 211642, 211643, 211644, 211645) c6(211646, 211647) c7(aC, fq, cz); #c1(NPW) c2(NP_001092926) c3(neuropeptide W preproprotein) c4(39778) c5(39779, 39780, 39781, 39782, 39783, 39784, 39785) c6( ) c7(bM, auq, clv, fD); #c1(NPY) c2(NP_000896) c3(pro-neuropeptide Y preproprotein) c4(16990) c5(16991, 16992, 16993, 16994, 16995, 16996, 16997, 16998, 16999, 17000, 17001, 17002, 17003, (7004) c6( ) c7(bM, T, vS, ya, ea, e, bO, ci, afl, aY, VV, aE, pX, M, Ou, K, qM, dQ, blc, xa, yo, aW, as, eE, cm, uC, clw, iT, Gs, U, vy, QF, sv, dD, lT, aw, n, To, aj, afE, ct, fr, ww, cd, rc, FY, kH, jy, gl, ey, bXV, he, dx, aze, A, cly, fq, bJ, hq, cA, dB, yP, Ei, JM, lB, bL, iH, eR, oe, qQ, cz, v, j, bX, iI, clx, eA, J, er, aPW, uT, aT, cy, ae, af, eu, ev, aKt, cu, en, FT, ck, pH, rR, ac, cS, VB, sY, iW, ha, vr, bS, dG, eq, hF, Fc, ad, Tg, w, ot, Sb, d, lR, tb, cH, ff, gC, gl, Tp, Z, FX, jb, qa, ek, TA, es, D, pu, Rm, aS, ei); #c1(NPY1R) c2(NP_000900) c3(neuropeptide Y receptor type 1) c4(17019, 17020, 17021, 17022, 17023) c5(17024, 17025, 17026, 17027, 17028, 17029, 17030, 17031, 17032, 17033, 17034, 17035, 17036, 17037, 17038, 17039, 17040, 17041, 17042, 17043, 17044, 17045, 17046, 17047, 17048, 17049, 17050, 17051, 17052, 17053) c6( ) c7(bL, aWm, ac, Bc, e, ha, hl, cz, cm, cu, sv, v, K, ct, iI, FX, xa, ad, vk, hq); #c1(NPY2R) c2(NP_000901) c3(neuropeptide Y receptor type 2) c4(17054) c5(17055, 17056, 17057, 17058, 17059, 17060, 17061, 17062, 17063, 17064) c6( ) c7(bL, oe, ha, qQ, bO, cz, v, iL, VV, ip, XY, cy, qM, jo, af, aW, cu, Tp, cm, FX, hq, rR, cA); #c1(NPY5R) c2(XP_011530316) c3(neuropeptide Y receptor type 5 isoform X2) c4( ) c5(326163, 326164, 326165) c6( ) c7(bL, rR, tb, lT, aW, iX, hl, aql, aT, iy, jy, cu, aj, v, ww, ei, FX, J, hq); #c1(NPY5R) c2(XP_011530317) c3(neuropeptide Y receptor type 5 isoform X3) c4(326166) c5( ) c6( ) c7(bL, rR, tb, lT, aW, iX, hl, aql, aT, iy, jy, cu, aj, v, ww, ei, FX, J, hq); #c1(NQO1) c2(NP_000894) c3(NAD(P)H dehydrogenase [quinone] 1 isoform a) c4(16983) c5(16984, 16985, 16986, 16987, 16988, 16989) c6( ) c7(bM, T, aC, pf, ea, if, e, bO, fd, bB, aeu, qj, ci, aJO, x, aE, M, ja, K, hu, pk, eL, ln, aa, Jl, aW, az, hl, LZ, nY, fc, g, hJ, cm, bw, fT, eM, nB, ii, iA, iT, Gs, cj, sv, dW, k, m, cE, iu, FW, gU, dD, aw, ht, n, aj, fr, qN, pM, cd, br, eW, b, ft, jz, ql, Bj, eV, Gw, dL, rk, A, sp, QP, aq, cM, Zj, iO, clz, kT, ok, cA, dB, anZ, at, eo, it, B, cn, bL, jv, yk, eX, aA, cz, hy, v, j, C, AO, V, q, J, eA, eJ, lg, Vr, aT, cy, dz, bq, an, ae, af, ne, s, iU, Y, r, akY, HL, en, t, iK, ck, ov, vK, sS, nM, ac, gX, dy, im, bt, Gl, wi, iW, jh, lD, dG, eq, fu, Fb, hF, iJ, ad, dP, av, HF, akN, ib, Db, ji, d, BN, qt, ff, gK, cr, aF, iV, FX, jb, bz, iq, es, bE, pP, gZ, Gu, i, X, us, pC); #c1(NQO1) c2(NP_001020604) c3(NAD(P)H dehydrogenase [quinone] 1 isoform b) c4(28955) c5(28956, 28957, 28958, 28959, 28960, 28961) c6( ) c7(bM, T, aC, pf, ea, if, e, b, fd, bB, aeu, qj, ci, aJO, x, aE, M, ja, K, hu, pk, eL, ln, aa, Jl, aW, az, hl, LZ, nY, fc, g, hJ, cm, bw, fT, eM, nB, ii, iA, iT, Gs, cj, sv, dW, k, m, cE, iu, FW, gU, dD, aw, ht, n, aj, fr, qN, pM, cd, br, eW, b, ft, jz, ql, Bj, eV, Gw, dL, rk, A, sp, QP, aq, cM, Zj, iO, clz, kT, ok, cA, dB, anZ, at, eo, it, B, cn, bL, jv, yk, eX, aA, cz, hy, v, j, C, AO, V, q, J, eA, eJ, lg, Vr, aT, cy, dz, bq, an, ae, af, ne, s, iU, Y, r, akY, HL, en, t, iK, ck, ov, vK, sS, nM, ac, gX, dy, im, bt, GI, wi, iW, jh, lD, dG, eq, fu, Fb, hF, iJ, ad, dP, av, HF, akN, ib, Db, ji, d, BN, qt, ff, gK, cr, aF, iV, FX, jb, bz, iq, es, bE, pP, gZ, Gu, i, X, us, pC); #c1(NQO1) c2(NP_001020605) c3 (NAD(P)H dehydrogenase [quinone] 1 isoform c) c4(28962) c5(28963, 28964, 28965, 28966, 28967, 28968) c6( ) c7(bM, T, aC, pf, ea, if, e, bO, fd, bB, aeu, qj, ci, aJO, x, aE, M, ja, K, hu, pk, eL, ln, aa, Jl, aW, az, hl, LZ, nY, fc, g, hJ, cm, bw, fT, eM, nB, ii, iA, iT, Gs, cj, sv, dW, k, m, cE, iu, FW, gU, dD, aw, ht, n, aj, fr, qN, pM, cd, br, eW, b, ft, jz, ql, Bj, eV, Gw, dL, rk, A, sp, QP, aq, cM, Zj, iO, clz, kT, ok, cA, dB, anZ, at, eo, it, B, cn, bL, jv, yk, eX, aA, cz, hy, v, j, C, AO, V, q, J, eA, eJ, lg, Vr, aT, cy, dz, bq, an, ae, af, ne, s, iU, Y, r, akY, HL, en, t, iK, ck, ov, vK, sS, nM, ac, gX, dy, im, bt, Gl, wi, iW, jh, lD, dG, eq, fu, Fb, hF, iJ, ad, dP, av, HF, akN, ib, Db, ji, d, BN, qt, ff, gK, cr, aF, iV, FX, jb, bz, iq, es, bE, pP, gZ, Gu, i, X, us, pC); #c1(NQO1) c2(NP_001273066) c3(NAD(P)H dehydrogenase [quinone] 1 isoform d) c4(104281) c5(104282, 104283, 104284, 104285, 104286, 104287) c6( ) c7(bM, T, aC, pf, ea, if, e, bO, fd, bB, aeu, qj, ci, aJO, x, aE, M, ja, K, hu, pk, eL, ln, aa, Jl, aW, az, hl, LZ, nY, fc, g, hJ, cm, bw, fT, eM, nB, ii, iA, iT, Gs, cj, sv, dW, k, m, cE, iu, FW, gU, dD, aw, ht, n, aj, fr, qN, pM, cd, br, eW, b, ft, jz, ql, Bj, eV, Gw, dL, rk, A, sp, QP, aq, cM, Zj, iO, clz, kT, ok, cA, dB, anZ, at, eo, it, B, cn, bL, jv, yk, eX, aA, cz, hy, v, j, C, AO, V, q, J, eA, eJ, lg, Vr, aT, cy, dz, bq, an, ae, af, ne, s, iU, Y, r, akY, HL, en, t, iK, ck, ov, vK, sS, nM, ac, gX, dy, im, bt, Gl, wi, iW, jh, lD, dG, eq, fu, Fb, hF, iJ, ad, dP, av, HF, akN, ib, Db, ji, d, BN, qt, ff, gK, cr, aF, iV, FX, jb, bz, iq, es, bE, pP, gZ, Gu, i, X, us, pC); #c1(NQO2) c2(NP_001277151) c3(ribosyldihydronicotinamide dehydrogenase [quinone] isoform 2) c4(109155, 109156) c5(109157, 109158, 109159, 109160, 109161, 109162, 109163, 109164, 109165, 109166, 109167, 109168, 109169, 109170, 109171, 109172) c6( ) c7(T, ht, iG, aR, ad, ot, aE, aT, dz, dL, K, hu, an, ae, PT, aW, Y, gl, aq, iR, t, FX, cm, hq, ck, iO, ok, bP, hA, dJ, Gs, aiS, m); #c1(NQO2) c2(XP 005249208) c3(ribosyldihydronicotinamide dehydrogenase [quinone] isoform X1) c4(269093, 269094) c5(269095, 269096, 269097, 269098, 269099, 269100, 269101, 269102, 269103, 269104, 269105, 269106, 269107, 269108) c6( ) c7(T, ht, iG, aR, ad, ot, aE, aT, dz, dL, K, hu, an, ae, PT, aW, Y, gl, aq, iR, t, FX, cm, hq, ck, iO, ok, bP, hA, dJ, Gs, aiS, m); #c1(NQO2) c2(XP_011512944) c3 (ribosyldihydronicotinamide dehydrogenase [quinone] isoform X2) c4(304378) c5(304379, 304380, 304381, 304382, 304383, 304384, 304385, 304386, 304387, 304388, 304389, 304390, 304391, 304392, 304393, 304394) c6( ) c7(T, ht, iG, aR, ad, ot, aE, aT, dz, dL, K, hu, an, ae, PT, aW, Y, gl, aq, iR, t, FX, cm, hq, ck, iO, ok, bP, hA, dJ, Gs, aiS, m); #c1(NROB1) c2(NP_000466) c3(nuclear receptor subfamily 0 group B member 1) c4(9265, 9266, 9267) c5(9268, 9269, 9270, 9271, 9272, 9273, 9274, 9275, 9276, 9277, 9278, 9279, 9280, 9281, 9282, 9283, 9284, 9285) c6( ) c7(bL, T, dD, aiK, aec, bbn, e, ht, ea, yk, cz, bB, bgj, n, v, eq, ci, nA, UI, oH, q, ad, er, UE, yn, iu, BI, eW, vJ, aX, BJ, bgm, aT, vH, UD, iv, ji, j, bUo, clD, hu, H, hK, ae, dx, cp, bgp, aeT, clA, yo, UL, lR, s, Qr, CN, aW, oV, fc, bCO, iw, r, cr, uY, aDm, baZ, g, iV, pt, GR, pb, bE, PI, AH, au, hL, clB, hR, at, nw, Xu, UF, lC, KS, clC, L); #c1(NR1D1) c2(NP_068370) c3(nuclear receptor subfamily 1 group D member 1) c4(209353) c5(209354, 209355, 209356, 209357, 209358, 209359) c6( ) c7(bM, T, dD, mS, eq, hd, mW, bfJ, aT, ta, bK, j, mY, mV, mU, IR, qt, aW, Z, t, mZ, mX, aPY, cA, nB, nw, m, mT); #c1(NR1D2) c2(NP_005117) c3(nuclear receptor subfamily 1 group D member 2 isoform 1) c4(153032, 153033) c5(153034, 153035, 153036, 153037, 153038, 153039, 153040, 153041, 153042, 153043, 153044, 153045) c6( ) c7(dP, gk, T, F, al, vi); #c1(NR1D2) c2(XP_006713514) c3(nuclear receptor subfamily 1 group D member 2 isoform X1) c4( ) c5(287183, 287184, 287185, 287186, 287187) c6( ) c7(dP, gk, T, F, al, vi); #c1(NR1H2) c2(NP_001243576) c3(oxysterols receptor LXR-beta isoform 2) c4(91086, 91087) c5(91088, 91089, 91090, 91091, 91092, 91093, 91094, 91095, 91096, 91097, 91098, 91099) c6( ) c7(bL, T, dD, pf, yk, iW, ha, ht, bB, n, v, Gn, ci, j, eq, Do, fr, q, ad, Vr, rA, aT, aYf, dL, KP, K, hu, ae, af, eL, aW, Y, aBq, ff, CT, r, La, en, cl, hJ, t, bw, bz, yX, ck, eM, bE, U, hL, m, bnB, B); #c1(NR1H2) c2(NP_009052) c3(oxysterols receptor LXR-beta isoform 1) c4(170454, 170455) c5(170456, 170457, 170458, 170459, 170460, 170461, 170462, 170463, 170464, 170465, 170466, 170467, 170468) c6( ) c7(bL, T, dD, pf, yk, iW, ha, ht, bB, n, v, Gn, ci, j, eq, Do, fr, q, ad, Vr, rA, aT, aYf, dL, KP, K, hu, ae, af, eL, aW, Y, aBq, ff, CT, r, La, en, cl, hJ, t, bw, bz, yX, ck, eM, bE, U, hL, m, bnB, B); #c1(NR1H3) c2(NP_001123573) c3(oxysterols receptor LXR-alpha isoform 2) c4(47629) c5(47630, 47631, 47632, 47633, 47634, 47635, 47636, 47637, 47638, 47639, 47640, 47641, 47642, 47643, 47644, 47645, 47646, 47647, 47648, 47649, 47650, 47651, 47652, 47653, 47654, 47655, 47656, 47657) c6( ) c7(bL, UH, dD, ea, iW, cz, v, j, eq, ad, l, u, h, K, ae, cH, cW, Bw, eH, cr, r, fc, dq, hJ, eF, aS, cP, ei, bt); #c1(NR1H3) c2(NP_001123574) c3(oxysterols receptor LXR-alpha isoform 3) c4(47658) c5(47659, 47660, 47661, 47662, 47663) c6( ) c7(bL, UH, dD, ea, iW, cz, v, j, eq, ad, l, u, h, K, ae, cH, cW, Bw, eH, cr, r, fc, dq, hJ, eF, aS, cP, ei, bt); #c1(NR1H3) c2(NP_005684) c3(oxysterols receptor LXR-alpha isoform 1) c4(158534, 158535, 158536, 158537) c5(158538, 158539, 158540, 158541, 158542, 158543, 158544, 158545, 158546, 158547, 158548, 158549) c6( ) c7(bL, UH, dD, ea, iW, cz, v, j, eq, ad, l, u, h, K, ae, cH, cW, Bw, eH, cr, r, fc, dq, hJ, eF, aS, cP, ei, bt); #c1(NR1H3) c2(XP_005252772) c3(oxysterols receptor LXR-alpha isoform X6) c4(272053, 272054) c5(272055, 272056, 272057, 272058, 272059, 272060, 272061, 272062, 272063, 272064, 272065, 272066) c6( ) c7(bL, UH, dD, ea, iW, cz, v, j, eq, ad, l, u, h, K, ae, cH, cW, Bw, eH, cr, r, fc, dq, hJ, eF, aS, cP, ei, bt); #c1(NR1H4) c2(NP_001193907) c3(bile acid receptor isoform 5) c4(83129) c5(83130, 83131, 83132, 83133, 83134, 83135) c6( ) c7(bL, T, jv, dD, fR, pf, al, NT, cz, v, j, eq, A, jw, hX, jF, l, aE, u, JD, M, aT, h, dL, ji, K, qM, bq, da, af, Qe, wC, UO, ls, ff, fc, hl, LZ, fe, dq, lo, akY, aq, Ku, en, hJ, g, iV, Jh, bbT, jq, ac, U, cj, uS, at, eo, B); #c1(NR1H4) c2(NP_001193908) c3(bile acid receptor isoform 1) c4(83136) c5(83137, 83138, 83139, 83140, 83141, 83142) c6( ) c7(bL, T, jv, dD, fR, pf, al, NT, cz, v, j, eq, AO, jw, hX, jF, l, aE, u, JD, M, aT, h, dL, ji, K, qM, bq, dQ, af, Qe, wC, UO, ls, ff, fc, hl, LZ, fe, dq, lo, akY, aq, Ku, en, hJ, g, iV, Jh, bbT, jq, ac, U, cj, uS, at, eo, B); #c1(NR1H4) c2(NP_001193921) c3(bile acid receptor isoform 4) c4(83156) c5(83157, 83158, 83159, 83160, 83161, 83162) c6( ) c7(bL, T, jv, dD, fR, pf, al, NT, cz, v, j, eq, AO, jw, hX, jF, l, aE, u, JD, M, aT, h, dL, ji, K, qM, bq, dQ, af, Qe, wC, UO, ls, ff, fc, hl, LZ, fe, dq, lo, akY, aq, Ku, en, hJ, g, iV, Jh, bbT, jq, ac, U, cj, uS, at, eo, B); #c1(NR1H4) c2(NP_001193922) c3(bile acid receptor isoform 3) c4(83163) c5(83164, 83165, 83166, 83167, 83168, 83169) c6( ) c7(bL, T, jv, dD, fR, pf, al, NT, cz, v, j, eq, AO, jw, hX, jF, l, aE, u, JD, M, aT, h, dL, ji, K, qM, bq, dQ, af, Qe, wC, UO, ls, ff, fc, hl, LZ, fe, dq, lo, akY, aq, Ku, en, hJ, g, iV, Jh, bbT, jq, ac, U, cj, uS, at, eo, B); #c1(NR1H4) c2(NP_005114) c3(bile acid receptor isoform 2) c4(153009) c5(153010, 153011, 153012, 153013, 153014, 153015, 153016) c6( ) c7(bL, T, jv, dD, fR, pf, al, NT, cz, v, j, eq, AO, jw, hX, jF, l, aE, u, JD, M, aT, h, dl, ji, K, qM, bq, dQ, af, Qe, wC, UO, ls, ff, fc, hl, LZ, fe, dq, lo, akY, aq, Ku, en, hJ, g, iV, Jh, bbT, jq, ac, U, cj, uS, at, eo, B); #c1(NR112) c2(NP_071285) c3(nuclear receptor subfamily 1 group 1 member 2 isoform 2) c4(210880, 210881) c5(210882, 210883, 210884, 210885, 210886, 210887, 210888, 210889, 210890, 210891, 210892, 210893, 210894, 210895) c6( ) c7(ID, gh, T, bM, aC, ea, e, al, fd, bB, ci, bv, aE, eL, bc, cp, aa, mH, aW, az, hl, LZ, fc, hN, alb, g, hJ, eE, aaD, eF, cm, zV, iA, bpY, acS, cj, cX, m, cE, aZN, NK, dD, aw, ht, ez, n, aj, fr, qN, jw, cd, ai, rc, ke, amB, jz, h, dL, bbN, wC, OV, auM, fe, aq, jq, kT, Pa, hk, hL, at, aiS, fL, agy, zB, it, B, bL, jv, dA, fG, aA, cR, hy, v, j, qf, C, sl, V, q, J, eA, ady, aT, cy, ae, af, ne, bA, nO, Y, ev, r, lo, eS, en, cl, t, iK, ck, wR, CZ, Tu, fD, gT, bt, ao, gV, Mb, ha, Jy, jh, wV, MC, oH, eq, mC, ad, BY, dP, hX, av, F, py, sN, vJ, ds, ji, lQ, d, BN, hG, ff, eH, cr, eZ, gl, dq, aF, iV, bz, bdu, LB, es, EW, yE, xZ, bE, uR, cP, ei, lC, ra, X, y); #c1(NR112) c2(NP_148934) c3(nuclear receptor subfamily 1 group 1 member 2 isoform 3) c4(226621) c5(226622, 226623, 226624, 226625, 226626, 226627, 226628) c6( ) c7(ID, gh, T, bM, aC, ea, e, al, fd, bB, ci, bv, aE, eL, bc, cp, aa, mH, aW, az, hl, LZ, fc, hN, alb, g, hJ, eE, aaD, eF, cm, zV, iA, bpY, acS, cj, cX, m, cE, aZN, NK, dD, aw, ht, ez, n, aj, fr, qN, jw, cd, ai, rc, ke, amB, jz, h, dL, bbN, wC, OV, auM, fe, aq, jq, kT, Pa, hk, hl, at, aiS, fL, agy, zB, it, B, bL, jv, dA, fG, aA, cR, hy, v, j, qf, C, sl, V, q, J, eA, ady, aT, cy, ae, af, ne, bA, n, Y, ev, r, lo, eS, en, cl, t, iK, ck, wR, CZ, Tu, fD, gT, bt, ao, gV, Mb, ha, Jy, jh, wV, MC, oH, eq, mC, ad, BY, dP, hX, av, F, py, sN, vJ, ds, ji, lQ, d, BN, hG, ff, eH, cr, eZ, gl, dq, aF, iV, bz, bdu, LB, es, EW, yE, xZ, bE, uR, cP, ei, lC, ra, X, y); #c1(NR113) c2(NP_001070937) c3(nuclear receptor subfamily 1 group 1 member 3 isoform 6) c4(34924) c5(34925, 34926, 34927, 34928, 34929, 34930) c6( ) c7(bL, gV, T, dA, jp, qb, NT, jh, fd, bB, aj, v, ci, C, jw, q, ad, eA, eW, aT, h, jy, dL, ae, af, eL, PP, s, hG, aW, Y, OX, oV, hC, r, aq, lo, as, hJ, t, cm, ck, kT, bE, mx, cj, mE, iS, MJ, m, ei, gT, bt, B, y); #c1(NR113) c2(NP_001070938) c3(nuclear receptor subfamily 1 group 1 member 3 isoform 11) c4(34931) c5(34932, 34933, 34934, 34935, 34936, 34937, 34938, 34939) c6( ) c7(bL, gV, T, dA, jp, qb, NT, jh, fd, bB, aj, v, ci, C, jw, q, ad, eA, eW, aT, h, jy, dL, ae, af, eL, PP, s, hG, aW, Y, OX, oV, hC, r, aq, lo, as, hJ, t, cm, ck, kT, bE, mx, cj, mE, iS, MJ, m, ei, gT, bt, B, y); #c1(NR113) c2(NP_001070939) c3(nuclear receptor subfamily 1 group 1 member 3 isoform 5) c4(34940) c5(34941, 34942, 34943, 34944, 34945, 34946) c6( ) c7(bL, gV, T, dA, jp, qb, NT, jh, fd, bB, aj, v, ci, C, jw, q, ad, eA, eW, aT, h, jy, dL, ae, af, eL, PP, s, hG, aW, Y, OX, oV, hC, r, aq, lo, as, hJ, t, cm, ck, kT, bE, mx, cj, mE, iS, MJ, m, ei, gT, bt, B, y); #c1(NR113) c2(NP_001070940) c3(nuclear receptor subfamily 1 group 1 member 3 isoform 9) c4(34947) c5(34948, 34949, 34950, 34951, 34952) c6( ) c7(bL, gV, T, dA, jp, qb, NT, jh, fd, bB, aj, v, ci, C, jw, q, ad, eA, eW, aT, h, jy, dL, ae, af, eL, PP, s, hG, aW, Y, OX, oV, hC, r, aq, lo, as, hJ, t, cm, ck, kT, bE, mx, cj, mE, iS, MJ, m, ei, gT, bt, B, y); #c1(NR113) c2(NP_001070941) c3(nuclear receptor subfamily 1 group 1 member 3 isoform (2) c4(34953, 34954) c5(34955, 34956, 34957, 34958, 34959, 34960, 34961, 34962, 34963, 34964, 34965) c6( ) c7(bL, gV, T, dA, jp, qb, NT, jh, fd, bB, aj, v, ci, C, jw, q, ad, eA, eW, aT, h, jy, dL, ae, af, eL, PP, s, hG, aW, Y, OX, oV, hC, r, aq, lo, as, hJ, t, cm, ck, kT, bE, mx, cj, mE, iS, MJ, m, ei, gT, bt, B, y); #c1(NR113) c2(NP_001070942) c3(nuclear receptor subfamily 1 group 1 member 3 isoform 8) c4(34966) c5(34967, 34968, 34969, 34970, 34971, 34972, 34973, 34974, 34975, 34976, 34977, 34978, 34979, 34980, 34981, 34982, 34983, 34984, 34985, 34986, 34987, 34988, 34989, 34990) c6( ) c7(bL, gV, T, dA, jp, qb, NT, jh, fd, bB, aj, v, ci, C, jw, q, ad, eA, eW, aT, h, jy, dL, ae, af, eL, PP, s, hG, aW, Y, OX, oV, hC, r, aq, lo, as, hJ, t, cm, ck, kT, bE, mx, cj, mE, iS, MJ, m, ei, gT, bt, B, y); #c1(NR113) c2(NP_001070943) c3(nuclear receptor subfamily 1 group 1 member 3 isoform 15) c4(34991) c5(34992, 34993, 34994, 34995, 34996, 34997, 34998, 34999, 35000, 35001, 35002, 35003, 35004, 35005, 35006, 35007, 35008, 35009, 35010, 35011, 35012, 35013, 35014, 35015, 35016, 35017, 35018, 35019) c6( ) c7(bL, gV, T, dA, jp, qb, NT, jh, fd, bB, aj, v, ci, C, jw, q, ad, eA, eW, aT, h, jy, dL, ae, af, eL, PP, s, hG, aW, Y, OX, oV, hC, r, aq, lo, as, hJ, t, cm, ck, kT, bE, mx, cj, mE, iS, MJ, m, ei, gT, bt, B, y); #c1(NR113) c2(NP_001070944) c3(nuclear receptor subfamily 1 group 1 member 3 isoform (3) c4(35020) c5(35021, 35022, 35023, 35024, 35025, 35026) c6( ) c7(bL, gV, T, dA, jp, qb, NT, jh, fd, bB, aj, v, ci, C, jw, q, ad, eA, eW, aT, h, jy, dL, ae, af, eL, PP, s, hG, aW, Y, OX, oV, hC, r, aq, lo, as, hJ, t, cm, ck, kT, bE, mx, cj, mE, iS, MJ, m, ei, gT, bt, B, y); #c1(NR113) c2(NP_001070945) c3(nuclear receptor subfamily 1 group 1 member 3 isoform 14) c4(35027) c5(35028, 35029, 35030, 35031, 35032, 35033) c6( ) c7(bL, gV, T, dA, jp, qb, NT, jh, fd, bB, aj, v, ci, C, jw, q, ad, eA, eW, aT, h, jy, dL, ae, af, eL, PP, s, hG, aW, Y, OX, oV, hC, r, aq, lo, as, hJ, t, cm, ck, kT, bE, mx, cj, mE, iS, MJ, m, ei, gT, bt, B, y); #c1(NR113) c2(NP_001070946) c3(nuclear receptor subfamily 1 group 1 member 3 isoform 7) c4(35034) c5(35035, 35036, 35037, 35038, 35039, 35040) c6( ) c7(bL, gV, T, dA, jp, qb, NT, jh, fd, bB, aj, v, ci, C, jw, q, ad, eA, eW, aT, h, jy, dL, ae, af, eL, PP, s, hG, aW, Y, OX, oV, hC, r, aq, lo, as, hJ, t, cm, ck, kT, bE, mx, cj, mE, iS, MJ, m, ei, gT, bt, B, y); #c1(NR113) c2(NP_001070947) c3(nuclear receptor subfamily 1 group 1 member 3 isoform 10) c4(35041, 35042, 35043) c5(35044, 35045, 35046, 35047, 35048, 35049) c6( ) c7(bL, gV, T, dA, jp, qb, NT, jh, fd, bB, aj, v, ci, C, jw, q, ad, eA, eW, aT, h, jy, dl, ae, af, eL, PP, s, hG, aW, Y, OX, oV, hC, r, aq, lo, as, hJ, t, cm, ck, kT, bE, mx, cj, mE, iS, MJ, m, ei, gT, bt, B, y); #c1(NR113) c2(NP_001070948) c3(nuclear receptor subfamily 1 group 1 member 3 isoform 2) c4(35050) c5(35051, 35052, 35053, 35054, 35055, 35056) c6( ) c7(bL, gV, T, dA, jp, qb, NT, jh, fd, bB, aj, v, ci, C, jw, q, ad, eA, eW, aT, h, jy, dL, ae, af, eL, PP, s, hG, aW, Y, OX, oV, hC, r, aq, lo, as, hJ, t, cm, ck, kT, bE, mx, cj, mE, iS, MJ, m, ei, gT, bt, B, y); #c1(NR113) c2(NP_001070949) c3(nuclear receptor subfamily 1 group 1 member 3 isoform 4) c4(35057) c5(35058, 35059, 35060, 35061, 35062, 35063, 35064) c6( ) c7(bL, gV, T, dA, jp, qb, NT, jh, fd, bB, aj, v, ci, C, jw, q, ad, eA, eW, aT, h, jy, dL, ae, af, eL, PP, s, hG, aW, Y, OX, oV, hC, r, aq, lo, as, hJ, t, cm, ck, kT, bE, mx, cj, mE, iS, MJ, m, ei, gT, bt, B, y); #c1(NR113) c2(NP_001070950) c3(nuclear receptor subfamily 1 group 1 member 3 isoform 1) c4(35065, 35066) c5(35067, 35068, 35069, 35070, 35071, 35072, 35073, 35074, 35075, 35076) c6( ) c7(bL, gV, T, dA, jp, qb, NT, jh, fd, bB, aj, v, ci, C, jw, q, ad, eA, eW, aT, h, jy, dL, ae, af, eL, PP, s, hG, aW, Y, OX, oV, hC, r, aq, lo, as, hJ, t, cm, ck, kT, bE, mx, cj, mE, iS, MJ, m, ei, gT, bt, B, y); #c1(NR113) c2(NP_005113) c3 (nuclear receptor subfamily 1 group 1 member 3 isoform 3) c4(153000, 153001) c5(153002, 153003, 153004, 153005, 153006, 153007, 153008) c6( ) c7(bL, gV, T, dA, jp, qb, NT, jh, fd, bB, aj, v, ci, C, jw, q, ad, eA, eW, aT, h, jy, dL, ae, af, eL, PP, s, hG, aW, Y, OX, oV, hC, r, aq, lo, as, hJ, t, cm, ck, kT, bE, mx, cj, mE, iS, MJ, m, ei, gT, bt, B, y); #c1(NR113) c2(XP_005245750) c3(nuclear receptor subfamily 1 group 1 member 3 isoform X1) c4( ) c5(266934, 266935) c6( ) c7(bL, gV, T, dA, jp, qb, NT, jh, fd, bB, aj, v, ci, C, jw, q, ad, eA, eW, aT, h, jy, dL, ae, af, eL, PP, s, hG, aW, Y, OX, oV, hC, r, aq, lo, as, hJ, t, cm, ck, kT, bE, mx, cj, mE, iS, MJ, m, ei, gT, bt, B, y); #c1(NR113) c2(XP_005245751) c3(nuclear receptor subfamily 1 group 1 member 3 isoform X2) c4( ) c5(266936) c6( ) c7(bL, gV, T, dA, jp, qb, NT, jh, fd, bB, aj, v, ci, C, jw, q, ad, eA, eW, aT, h, jy, dL, ae, af, eL, PP, s, hG, aW, Y, OX, oV, hC, r, aq, lo, as, hJ, t, cm, ck, kT, bE, mx, cj, mE, iS, MJ, m, ei, gT, bt, B, y); #c1(NR113) c2(XP_005245754) c3(nuclear receptor subfamily 1 group 1 member 3 isoform X4) c4(266937) c5( ) c6( ) c7(bL, gV, T, dA, jp, qb, NT, jh, fd, bB, aj, v, ci, C, jw, q, ad, eA, eW, aT, h, jy, dL, ae, af, eL, PP, s, hG, aW, Y, OX, oV, hC, r, aq, lo, as, hJ, t, cm, ck, kT, bE, mx, cj, mE, iS, MJ, m, ei, gT, bt, B, y); #c1(NR(13) c2(XP_011508539) c3(nuclear receptor subfamily 1 group 1 member 3 isoform X3) c4( ) c5(299235, 299236) c6( ) c7(bL, gV, T, dA, jp, qb, NT, jh, fd, bB, aj, v, ci, C, jw, q, ad, eA, eW, aT, h, jy, dL, ae, af, eL, PP, s, hG, aW, Y, OX, oV, hC, r, aq, lo, as, hJ, t, cm, ck, kT, bE, mx, cj, mE, iS, MJ, m, ei, gT, bt, B, y); #c1(NR2C1) c2(NP_001027458) c3(nuclear receptor subfamily 2 group C member 1 isoform b) c4(30208) c5(30209, 30210, 30211, 30212, 30213, 30214) c6( ) c7(dP, ae, ad); #c1(NR2C1) c2(NP_001120834) c3(nuclear receptor subfamily 2 group C member 1 isoform c) c4(44811) c5(44812, 44813, 44814, 44815, 44816, 44817) c6( ) c7(dP, ae, ad); #c1(NR2C1) c2(NP_003288) c3(nuclear receptor subfamily 2 group C member 1 isoform a) c4(137160) c5(137161, 137162, 137163, 137164, 137165, 137166, 137167) c6( ) c7(dP, ae, ad); #c1(NR2C1) c2(XP_006719649) c3(nuclear receptor subfamily 2 group C member 1 isoform X6) c4(292867) c5( ) c6( ) c7(dP, ae, ad); #c1(NR2C2) c2(NP_001278623) c3(nuclear receptor subfamily 2 group C member 2 isoform 2) c4( ) c5(109891, 109892, 109893, 109894, 109895, 109896, 109897, 109898, 109899, (09900, 109901, 109902) c6( ) c7(T, bsy, lH, bB, aNG, ci, Lw, ad, er, dP, Na, aE, bjA, aT, dL, ji, ae, eL, B, aW, Y, aq, si, t, bz, ck, cbL, m, bt, bsw, vQ); #c1(NR2C2) c2(NP_003289) c3(nuclear receptor subfamily 2 group C member 2 isoform 1) c4(137168) c5(137169, 137170, 137171, 137172, 137173, 137174, 137175, 137176, 137177, 137178) c6( ) c7(T, bsy, lH, bB, aNG, ci, Lw, ad, er, dP, Na, aE, bjA, aT, dL, ji, ae, eL, B, aW, Y, aq, si, t, bz, ck, cbL, m, bt, bsw, vQ); #c1(NR2C2) c2(XP_011532363) c3 (nuclear receptor subfamily 2 group C member 2 isoform X2) c4(328537, 328538) c5(328539, 328540, 328541, 328542, 328543, 328544, 328545) c6( ) c7(T, bsy, lH, bB, aNG, ci, Lw, ad, er, dP, Na, aE, bjA, aT, dL, ji, ae, eL, B, aW, Y, aq, si, t, bz, ck, cbL, m, bt, bsw, vQ); #c1(NR2C2) c2(XP_011532364) c3(nuclear receptor subfamily 2 group C member 2 isoform X3) c4( ) c5(328546, 328547) c6( ) c7(T, bsy, lH, bB, aNG, ci, Lw, ad, er, dP, Na, aE, bjA, aT, dL, ji, ae, eL, B, aW, Y, aq, si, t, bz, ck, cbL, m, bt, bsw, vQ); #c1(NR2C2) c2(XP_011532365) c3(nuclear receptor subfamily 2 group C member 2 isoform X4) c4(328548) c5(328549, 328550) c6( ) c7(T, bsy, lH, bB, aNG, ci, Lw, ad, er, dP, Na, aE, bjA, aT, dL, ji, ae, eL, B, aW, Y, aq, si, t, bz, ck, cbL, m, bt, bsw, vQ); #c1(NRZE1) c2(NP_001273031) c3(nuclear receptor subfamily 2 group E member 1 isoform a) c4(104193) c5(104194, 104195, 104196, 104197, 104198, 104199, 104200) c6( ) c7(QK, dB, ff, dy, bK, en, he, FI); #c1(NRZE1) c2(NP_003260) c3(nuclear receptor subfamily 2 group E member 1 isoform b) c4(136902) c5(136903, 136904, 136905, 136906, 136907, 136908, 136909) c6( ) c7(QK, dB, ff, dy, bK, en, he, FI); #c1(NRZE1) c2(XP_011534396) c3(nuclear receptor subfamily 2 group E member 1 isoform X1) c4(330778) c5(330779, 330780, 330781, 330782, 330783) c6( ) c7(QK, dB, ff, dy, bK, en, he, Fl); #c1(NR2E3) c2(NP_055064) c3 (photoreceptor-specific nuclear receptor isoform b) c4(178689, 178690) c5(178691) c6( ) c7(clH, clF, T, xX, lR, W, bFo, clE, nx, aW, cII, aT, n, nw, nK, clG, nC, ayj, de); #c1(NR2E3) c2(NP_057430) c3(photoreceptor-specific nuclear receptor isoform a) c4( ) c5(190766, 190767, 190768, 190769, 190770, 190771) c6( ) c7(clH, clF, T, xX, lR, W, bFo, clE, nx, aW, cII, al, n, nw, nK, clG, nC, ayj, de); #c1(NRZE3) c2(XP_011519448) c3(photoreceptor-specific nuclear receptor isoform X1) c4( ) c5(314119, 314120, 314121, 314122, 314123) c6( ) c7(clH, cIF, T, XX, lR, W, bFo, clE, nx, aW, cII, aT, n, nw, nK, clG, nC, ayj, de); #c1(NR2F1) c2(NP_005645) c3(COUP transcription factor 1) c4(158235) c5(158236, 158237, 158238, 158239, 158240, 158241, 158242) c6( ) c7(T, jv, cz, ci, nA, V, q, ad, aT, ae, clJ, s, aW, r, t, uC, op, vA, awK, m, bD, vi); #c1(NR2F2) c2(NP_001138627) c3(COUP transcription factor 2 isoform b) c4(57261) c5(57262, 57263, 57264, 57265, 57266, 57267, 57268, 57269) c6( ) c7(bL, T, dD, BC, BD, aC, ea, cz, bB, n, fd, bKx, j, ci, nA, eq, hF, V, AO, q, ad, er, eJ, dP, aE, aT, h, aAG, ae, A, aW, oV, aNb, ev, r, aq, t, bz, op, ck, bE, vA, wk, awK, m, bD, y, B); #c1(NR2F2) c2(NP_001138629) c3(COUP transcription factor 2 isoform c) c4(57270) c5(57271, 57272, 57273, 57274, 57275, 57276, 57277, 57278) c6( ) c7(bL, T, dD, BC, BD, aC, ea, cz, bB, n, fd, bKx, j, ci, nA, eq, hF, V, AO, q, ad, er, eJ, dP, aE, aT, h, aAG, ae, A, aW, oV, aNb, ev, r, aq, t, bz, op, ck, bE, vA, wk, awK, m, bD, y, B); #c1(NR2F2) c2(NP_066285) c3(COUP transcription factor 2 isoform a) c4(206990) c5(206991, 206992, 206993, 206994, 206995, 206996, 206997) c6( ) c7(bL, T, dD, BC, BD, aC, ea, cz, bB, n, fd, bKx, j, ci, nA, eq, hF, V, AO, q, ad, er, eJ, dP, aE, aT, h, aAG, ae, A, aW, oV, aNb, ev, r, aq, t, bz, op, ck, bE, vA, wk, awK, m, bD, y, B); #c1(NR2F6) c2(NP_005225) c3(nuclear receptor subfamily 2 group F member 6) c4(154208, 154209) c5(154210, 154211, 154212, 154213, 154214, 154215, 154216, 154217, 154218, 154219, 154220, 154221) c6( ) c7(dP, T, bE, r, bB, aT, q); #c1(NR3C1) c2(NP_001018085) c3 (glucocorticoid receptor isoform alpha) c4(27169, 27170, 27171, 27172) c5(27173, 27174, 27175, 27176, 27177, 27178, 27179, 27180, 27181, 27182, 27183, 27184, 27185, 27186, 27187, 27188, 27189, 27190, 27191, 27192, 27193) c6( ) c7(gh, T, bM, FR, vf, e, lr, bO, bB, fd, gf, ci, ip, GK, x, tg, xE, oG, axy, vg, Ds, K, qM, hu, wt, DY, eL, aa, bo, eG, yo, amy, aW, wF, Fa, uY, g, ir, hJ, eF, cm, mZ, ou, eM, Gs, cj, uo, uJ, OD, k, m, xh, dD, Uc, sQ, aVR, aw, ht, n, aj, afE, ct, fr, bLU, qW, cd, eW, ft, ec, Bj, adX, cU, jy, h, hn, dL, bI, aAG, uV, mV, mY, he, aDp, cIK, cC, hi, mU, Rj, lb, ap, ge, Xg, aq, fe, Hu, yq, ajo, gG, dw, pm, mX, bJ, hq, ok, cA, adZ, JQ, yP, Ei, mg, Uv, vL, hR, ut, at, pg, fL, B, mT, aOo, bL, Dw, pF, eR, AL, DD, oe, aA, cz, mS, fF, v, j, OY, C, aql, Qo, iI, V, wy, eA, J, clN, oT, bkJ, mP, clO, aVM, aql, aT, wP, bK, hK, ae, bZN, bA, eu, qw, hM, kl, s, aeS, r, cu, lo, cfm, en, t, ck, vK, sn, kP, ac, clL, pZ, mf, fD, lw, xb, LS, VB, ao, qX, id, aZ, rQ, iW, oA, Jy, jh, bS, ah, jE, eq, adg, OH, clM, ad, qC, dP, av, py, vJ, mW, Ot, Dq, lQ, jo, uQ, gj, lR, cH, cW, bPq, ff, eH, Ce, eZ, gl, dq, aeV, hp, aF, iV, Z, bz, dr, qa, ek, TA, pc, yE, bE, ei, pk, y); #c1(NR3C1) c2(NP_001018661) c3(glucocorticoid receptor isoform beta) c4(27529, 27530, 27531) c5(27532, 27533, 27534, 27535, 27536, 27537) c6( ) c7(gh, T, bM, fR, vf, e, lr, bO, bB, fd, gf, ci, ip, GK, x, tg, xE, oG, axy, vg, Ds, K, qM, hu, wt, DY, eL, aa, bo, eG, yo, amy, aW, wf, Fa, uY, g, ir, hJ, eF, cm, mZ, ou, eM, Gs, cj, uo, uJ, OD, k, m, xh, dD, Uc, sQ, aVR, aw, ht, n, aj, afE, ct, fr, bLU, qW, cd, eW, ft, ec, Bj, adX, cU, jy, h, hn, dL, bl, aAG, uV, mV, mY, he, aDp, cIK, cC, hi, mU, Rj, lb, ap, ge, Xg, aq, fe, Hu, yq, ajo, gG, dw, pm, mX, bJ, hq, ok, cA, adZ, JQ, yP, Ei, mg, Uv, vL, hR, ut, at, pg, fL, B, mT, aOo, bL, Dw, pF, eR, AL, DD, oe, aA, cz, mS, fF, v, j, OY, C, aql, Qo, iL, V, wy, eA, J, clN, oT, bkJ, mP, clO, aVM, aql, aT, wP, bK, hK, ae, bZN, bA, eu, qw, hM, kl, s, aeS, r, cu, lo, cfm, en, t, ck, vK, sn, kP, ac, ciL, pZ, mf, fD, lw, xb, LS, VB, ao, qX, id, aZ, rQ, iW, oA, Jy, jh, bS, ah, jE, eq, adg, OH, clM, ad, qC, dP, av, py, vJ, mW, Ot, Dq, lQ, jo, uQ, gj, lR, cH, cW, bPq, ff, eH, Ce, eZ, gl, dq, aeV, hp, aF, iV, Z, bz, dr, qa, ek, TA, pc, yE, bE, ei, pk, y); #c1(NR3C1) c2(NP_001019265) c3(glucocorticoid receptor isoform gamma) c4(27733) c5(27734, 27735, 27736, 27737, 27738, 27739, 27740, 27741, 27742, 27743, 27744, 27745, 27746, 27747) c6( ) c7(gh, T, bM, fR, vf, e, lr, bQ, bB, fd, gf, ci, ip, GK, x, tg, xE, oG, axy, vg, Ds, K, qM, hu, wt, DY, eL, aa, bo, eG, yo, amy, aW, wf, Fa, uY, g, ir, hJ, eF, cm, mZ, ou, eM, Gs, cj, uo, uJ, OD, k, m, xh, dD, Uc, sQ, aVR, aw, ht, n, aj, afE, ct, fr, bLU, qW, cd, eW, ft, ec, Bj, adX, cU, jy, h, hn, dL, bI, aAG, uV, mV, mY, he, aDp, cIK, cC, hi, mU, Rj, lb, ap, ge, Xg, aq, fe, Hu, yq, ajo, gG, dw, pm, mX, bJ, hq, ok, cA, adZ, JQ, yP, Ei, mg, Uv, vL, hR, ut, at, pg, fL, B, mT, aOo, bL, Dw, pF, eR, AL, DD, oe, aA, cz, mS, fF, v, j, OY, C, aql, Qo, iI, V, wy, eA, J, clN, oT, bkJ, mP, clO, aVM, aql, aT, wP, bK, hK, ae, bZN, bA, eu, qw, hM, kl, s, aeS, r, cu, lo, cfm, en, t, ck, vK, sn, kP, ac, ciL, pZ, mf, fD, lw, xb, LS, VB, ao, qX, id, aZ, rQ, iW, oA, Jy, jh, bS, ah, jE, eq, adg, OH, clM, ad, qC, dP, av, py, vJ, mW, Ot, Dq, lQ, jo, uQ, gj, lR, cH, cW, bPq, ff, eH, Ce, eZ, gl, dq, aeV, hp, aF, iV, Z, bz, dr, qa, ek, TA, pc, yE, bE, ei, pk, y); #c1(NR3C1) c2(NP_001191194) c3(glucocorticoid receptor isoform GR-P) c4( ) c5(81101, 81102, 81103, 81104, 81105, 81106) c6( ) c7(gh, T, bM, fR, vf, e, lr, bO, bB, fd, gf, ci, ip, GK, x, tg, xE, oG, axy, vg, Ds, K, qM, hu, wt, DY, eL, aa, bo, eG, yo, amy, aW, wf, Fa, uY, g, ir, hJ, eF, cm, mZ, ou, eM, Gs, cj, uo, uJ, OD, k, m, xh, dD, Uc, sQ, aVR, aw, ht, n, aj, afE, ct, fr, bLU, qW, cd, eW, ft, ec, Bj, adX, cU, jy, h, hn, dL, bl, aAG, uV, mV, mY, he, aDp, clk, cC, hi, mU, Rj, lb, ap, ge, Xg, aq, fe, Hu, yq, ajo, gG, dw, pm, mX, bJ, hq, ok, cA, adZ, JQ, yP, Ei, mg, Uv, vL, hR, ut, at, pg, fL, B, mT, aOo, bL, Dw, pF, eR, AL, DD, oe, aA, cz, mS, fF, v, j, OY, C, agl, Qo, iL, V, wy, eA, J, clN, oT, bkJ, mP, clO, aVM, aql, aT, wP, bK, hK, ae, bZN, bA, eu, qw, hM, kl, s, aeS, r, cu, lo, cfm, en, t, ck, vK, sn, kP, ac, clL, pZ, mf, fD, lw, xb, LS, VB, ao, qX, id, aZ, rQ, iW, DA, Jy, jh, bS, ah, jE, eq, adg, OH, clM, ad, qC, dP, av, py, vJ, mW, Ot, Dq, lQ, jo, uQ, gj, lR, cH, cW, bPq, ff, eH, Ce, eZ, gl, dq, aeV, hp, aF, iV, Z, bz, dr, qa, ek, TA, pc, yE, bE, ei, pK, y); #c1(NR3C1) c2(XP_011535938) c3(glucocorticoid receptor isoform X2) c4( ) c5(333198, 333199, 333200, 333201, 333202, 333203, 333204, 333205, 333206, 333207, 333208, 333209, 333210, 333211, 333212, 333213, 333214, 333215, 333216, 333217, 333218, 333219, 333220, 333221) c6( ) c7(gh, T, bM, fR, vf, e, lr, bO, bB, fd, gf, ci, ip, GK, x, tg, xE, oG, axy, vg, Ds, K, qM, hu, wt, DY, eL, aa, bo, eG, yo, amy, aW, wf, Fa, uY, g, ir, hJ, eF, cm, mZ, ou, eM, Gs, cj, uo, uJ, OD, k, m, xh, dD, Uc, sQ, aVR, aw, ht, n, aj, afE, ct, fr, bLU, qW, cd, eW, ft, ec, Bj, adX, cU, jy, h, hn, dL, bl, aAG, uV, mV, mY, he, aDp, clk, cC, hi, mU, Rj, lb, ap, ge, Xg, aq, fe, Hu, yq, ajo, gG, dw, pm, mX, bJ, hq, ok, cA, adZ, JA, yP, Ei, mg, Uv, vL, hR, ut, at, pg, fl, B, mT, aOo, bL, Dw, pF, eR, AL, DD, oe, aA, cz, mS, fF, v, j, OY, C, aql, Qo, iI, V, wy, eA, J, clN, oT, bkJ, mP, clO, aVM, aql, aT, wP, bK, hK, ae, bZN, bA, eu, qw, hM, kl, s, aeS, r, cu, lo, cfm, en, t, ck, vK, sn, kP, ac, ciL, pZ, mf, fD, lw, xb, LS, VB, ao, qX, id, aZ, rQ, iW, oA, Jy, jh, bS, ah, jE, eq, adg, OH, clM, ad, qC, dP, av, py, vJ, mW, Ot, Dq, lQ, jo, uQ, gj, lR, cH, cW, bPq, ff, eH, Ce, eZ, gl, dq, aeV, hp, aF, iV, Z, bz, dr, qa, ek, TA, pc, yE, bE, ei, pk, y); #c1(NR3C1) c2(XP_011535939) c3(glucocorticoid receptor isoform X3) c4( ) c5(333222, 333223, 333224, 333225, 333226, 333227) c6( ) c7(gh, T, bM, fR, vf, e, lr, bO, bB, fd, gf, ci, ip, GK, x, tg, xE, oG, axy, vg, Ds, K, qM, hu, wt, DY, eL, aa, bo, eG, yo, amy, aW, wF, Fa, uY, g, ir, hJ, eF, cm, mZ, ou, eM, Gs, cj, uo, uJ, OD, k, m, xh, dD, Uc, sQ, aVR, aw, ht, n, aj, afE, ct, fr, bLU, qW, cd, eW, ft, ec, Bj, adX, cU, jy, h, hn, dL, bI, aAG, uV, mV, mY, he, aDp, cIK, cC, hi, mU, Rj, lb, ap, ge, Xg, aq, fe, Hu, yq, ajo, gG, dw, pm, mX, bJ, hq, ok, cA, adZ, JQ, yP, Ei, mg, Uv, vL, hR, ut, at, pg, fL, B, mT, aOo, bL, Dw, pF, eR, AL, DD, oe, aA, cz, mS, fF, v, j, OY, C, aql, Qo, iL, V, wy, eA, J, clN, oT, bkJ, mP, clO, aVM, aql, aT, wP, bK, hK, ae, bZN, bA, eu, qw, hM, kl, s, aeS, r, cu, lo, cfm, en, t, ck, vK, sn, kP, ac, ciL, pZ, mf, fD, lw, xb, LS, VB, ao, qX, id, aZ, rQ, iW, oA, Jy, jh, bS, ah, jE, eq, adg, OH, clM, ad, qC, dP, av, py, vJ, mW, Ot, Dq, lQ, jo, uQ, gj, lR, cH, cW, bPq, ff, eH, Ce, eZ, gl, dq, aeV, hp, aF, iV, Z, bz, dr, qa, ek, TA, pc, yE, bE, ei, pK, y); #c1(NR3C2) c2(NP_000892) c3(mineralocorticoid receptor isoform 1) c4(16964, (6965) c5(16966, 16967, 16968, 16969, 16970, 16971, 16972, 16973, 16974, 16975, 16976, 16977, 16978, 16979, 16980, 16981, 16982) c6( ) c7(T, ahS, ea, e, al, lr, eb, bB, bO, gf, ci, aY, clR, M, K, wt, eL, Fe, Fa, uC, mx, cj, lS, dD, bLl, aw, kM, n, fr, ct, qW, fK, cd, cc, dL, he, Gh, fq, hq, cA, bo, clS, MM, jd, aOo, mr, DD, rf, cz, cR, v, aql, V, q, aT, cy, bK, Fg, iI, r, ul, pH, ac, wl, dh, fP, mf, dy, clP, clQ, bS, eq, hF, dP, fU, sN, gj, Z, dr, qa, ek, bF, bE, gZ, aS, ei, hE, gq); #c1(NR3C2) c2(NP_001159576) c3(mineralocorticoid receptor isoform 2) c4(64309, 64310) c5(64311, 64312, 64313, 64314, 64315, 64316, 64317, 64318, 64319, 64320, 64321, 64322) c6( ) c7(T, ahS, ea, e, al, lr, eb, bB, bO, gf, ci, aY, clR, M, K, wt, eL, Fe, Fa, uC, mx, cj, lS, dD, bLl, aw, kM, n, fr, ct, qW, fK, cd, cc, dL, he, Gh, fq, hq, cA, bo, clS, MM, jd, aOo, mr, DD, rf, cz, cR, v, aql, V, q, aT, cy, bK, Fg, iU, r, uU, pH, ac, wO, dh, fP, mf, dy, clP, clQ, bS, eq, hF, dP, fU, sN, gj, Z, dr, qa, ek, bF, bE, gZ, aS, ei, hE, gq); #c1(NR3C2) c2(XP_011530279) c3(mineralocorticoid receptor isoform X1) c4(326136) c5(326137, 326138, 326139, 326140, 326141, 326142, 326143, 326144, 326145, 326146) c6( ) c7(T, ahS, ea, e, al, lr, eb, bB, bO, gf, ci, aY, clR, M, K, wt, eL, Fe, Fa, uC, mx, cj, lS, dD, bLl, aw, kM, n, fr, ct, qW, fK, cd, cc, dL, he, Gh, fq, hq, cA, bo, clS, MM, jd, aOo, mr, DD, rf, cz, cR, v, aql, V, q, aT, cy, bK, Fg, iU, r, ul, pH, ac, wO, dh, fP, mf, dy, clP, clQ, bS, eq, hF, dP, fU, sN, gj, Z, dr, qa, ek, bF, bE, gZ, aS, ei, hE, gq); #c1(NR4A1) c2(NP_001189162) c3(nuclear receptor subfamily 4 group A member 1 isoform 2) c4(79242) c5(79243, 79244, 79245, 79246, 79247, 79248, 79249, 79250, 79251, 79252, 79253, 79254, 79255, 79256, 79257, 79258, 79259, 79260, 79261, 79262, 79263, 79264, 79265) c6( ) c7(bL, T, dD, fM, dA, ajm, aC, ahS, ea, jh, aA, bB, v, j, ci, eq, fr, hF, V, q, ad, hX, dP, av, cF, hd, aE, aT, bK, K, ae, B, aW, Y, ff, r, aDe, dq, aq, aF, HL, en, iR, bz, op, ck, qJ, kT, dB, bE, vA, cj, aS, y, bt, X); #c1(NR4A1) c2(NP_001189163) c3(nuclear receptor subfamily 4 group A member 1 isoform 3) c4(79266, 79267) c5(79268, 79269, 79270, 79271, 79272, 79273, 79274, 79275) c6( ) c7(bL, T, dD, fM, dA, ajm, aC, ahS, ea, jh, aA, bB, v, j, ci, eq, fr, hF, V, q, ad, hX, dP, av, cF, hd, aE, aT, bk, K, ae, B, aW, Y, ff, r, aDe, dq, aq, aF, HL, en, iR, bz, op, ck, qJ, kT, dB, bE, vA, cj, aS, y, bt, X); #c1(NR4A1) c2(XP_011536553) c3(nuclear receptor subfamily 4 group A member 1 isoform X3) c4(334032) c5(334033, 334034, 334035, 334036, 334037, 334038) c6( ) c7(bL, T, dD, fM, dA, ajm, aC, ahS, ea, jh, aA, bB, v, j, ci, eq, fr, hF, V, q, ad, hX, dP, av, cF, hd, aE, aT, bK, K, ae, B, aW, Y, ff, r, aDe, dq, aq, aF, HL, en, iR, bz, op, ck, qJ, kT, dB, bE, vA, cj, aS, y, bt, X); #c1(NR4A2) c2(NP_006177) c3(nuclear receptor subfamily 4 group A member 2) c4(162588, 162589, 162590) c5(162591, 162592, 162593, 162594, 162595, 162596, 162597, 162598, 162599, 162600, 162601, 162602, 162603, 162604, 162605, 162606, 162607) c6( ) c7(T, iB, e, bO, aA, bB, v, V, q, Pm, ai, wD, Hx, aqk, aT, iv, dL, bK, K, IQ, an, Tj, hZ, ak, aWm, Di, s, Jl, aW, Y, fO, cr, r, aF, g, Eg, cm, bz, op, GB, kT, bE, vA, agl, eo, lS, act, Pk, B, y); #c1(NR4A2) c2(XP_005246678) c3(nuclear receptor subfamily 4 group A member 2 isoform X1) c4(267326, 267327) c5(267328, 267329, 267330, 267331, 267332, 267333) c6( ) c7(T, iB, e, bO, aA, bB, v, V, q, Pm, ai, wD, Hx, aqk, aT, iv, dL, bK, K, lQ, an, Tj, hZ, ak, aWm, Di, s, Jl, aW, Y, fO, cr, r, aF, g, Eg, cm, bz, op, GB, kT, bE, vA, agl, eo, lS, act, Pk, B, y); #c1(NR4A2) c2(XP_005246680) c3(nuclear receptor subfamily 4 group A member 2 isoform X4) c4( ) c5(267334, 267335, 267336, 267337, 267338, 267339, 267340, 267341) c6( ) c7(T, iB, e, bO, aA, bB, v, V, q, Pm, ai, wD, Hx, aqk, aT, iv, dL, bK, K, lQ, an, Tj, hZ, ak, aWm, Di, s, Jl, aW, Y, fO, cr, r, aF, g, Eg, cm, bz, op, GB, kT, bE, vA, agl, eo, lS, act, Pk, B, y); #c1(NR4A2) c2(XP_011509548) c3(nuclear receptor subfamily 4 group A member 2 isoform X3) c4( ) c5(300127, 300128, 300129, 300130, 300131, 300132, 300133, 300134) c6( ) c7(T, iB, e, bO, aA, bB, v, V, q, Pm, ai, wD, Hx, aqk, aT, iv, dL, bK, K, lQ, an, Tj, hZ, ak, aWm, Di, s, Jl, aW, Y, fO, cr, r, aF, g, Eg, cm, bz, op, GB, kT, bE, vA, agl, eo, lS, act, Pk, B, y); #c1(NR4A3) c2(NP_008912) c3(nuclear receptor subfamily 4 group A member 3 isoform a) c4(169554) c5( ) c6( ) c7(bL, bM, T, kh, dD, xu, lD, aNA, ea, aHA, yG, ha, cz, aj, v, eq, ci, fr, atN, aY, pd, br, J, ad, Na, ec, M, aT, FQ, Nc, bK, K, yh, ae, azh, ly, hM, A, xU, cH, aW, Y, vj, Zf, Gv, Mz, aDP, cm, bw, KM, qa, lx, op, LB, ck, QG, Qf, eM, iT, Nk, vA, aiM, ajd, cj, clT, CZ, y, bt, ZW, B); #c1 (NR4A3) c2(NP_775291) c3(nuclear receptor subfamily 4 group A member 3 isoform c) c4(249851, 249852) c5(249853, 249854, 249855, 249856, 249857, 249858, 249859, 249860, 249861, 249862, 249863, 249864) c6( ) c7(bL, bM, T, kh, dD, xu, lD, aNA, ea, aHA, yG, ha, cz, aj, v, eq, ci, fr, atN, aY, pd, br, J, ad, Na, ec, M, aT, FQ, Nc, bK, K, yh, ae, azh, ly, hM, A, xU, cH, aW, Y, vj, Zf, Gv, Mz, aDP, cm, bw, KM, qa, lx, op, LB, ck, QG, Qf, eM, iT, Nk, vA, aiM, ajd, cj, clT, CZ, y, bt, ZW, B); #c1(NR4A3) c2(NP_775292) c3(nuclear receptor subfamily 4 group A member 3 isoform b) c4(249865) c5(249866, 249867, 249868, 249869, 249870, 249871, 249872, 249873, 249874, 249875, 249876, 249877, 249878) c6( ) c7(bL, bM, T, kh, dD, xu, lD, aNA, ea, aHA, yG, ha, cz, aj, v, eq, ci, fr, atN, aY, pd, br, J, ad, Na, ec, M, aT, FQ, Nc, bK, K, yh, ae, azh, ly, hM, A, xU, CH, aW, Y, vj, Zf, Gv, Mz, aDP, cm, bw, KM, qa, lx, op, LB, ck, QG, Qf, eM, iT, Nk, vA, aiM, ajd, cj, clT, CZ, y, bt, ZW, B); #c1(NR4A3) c2(XP_005252294) c3(nuclear receptor subfamily 4 group A member 3 isoform X2) c4( ) c5(271653) c6( ) c7(bL, bM, T, kh, dD, xu, lD, aNA, ea, aHA, yG, ha, cz, aj, v, eq, ci, fr, atN, aY, pd, br, J, ad, Na, ec, M, aT, FQ, Nc, bK, K, yh, ae, azh, ly, hM, A, xU, cH, aW, Y, vj, Zf, Gv, Mz, aDP, cm, bw, KM, qa, lx, op, LB, ck, QG, Qf, eM, iT, Nk, vA, aiM, ajd, cj, clT, CZ, y, bt, ZW, B); #c1(NR5A1) c2(NP_004950) c3(steroidogenic factor 1) c4(151409, 151410) c5(151411, 151412, 151413, 151414, 151415, 151416, 151417) c6( ) c7(bL, T, gU, bbn, adW, n, aj, bS, v, ct, Ns, ci, eA, J, er, dP, PW, clU, aX, vJ, il, PD, vH, UD, iv, bUo, hk, Fg, bgp, aeT, yo, yi, UL, s, ND, bfg, asP, vT, asQ, iw, bbs, dq, Pp, Lc, Hu, Z, t, clV, zZ, hq, ck, hL, KZ, m, Ux, UF, Po); #c1 (NR5A1) c2(XP_005251929) c3(steroidogenic factor 1 isoform X2) c4(271300) c5(271301, 271302, 271303, 271304, 271305, 271306) c6( ) c7(bL, T, gU, bbn, adW, n, aj, bS, v, ct, Ns, ci, eA, J, er, dP, PW, clU, aX, vJ, il, PD, vH, UD, iv, bUo, hK, Fg, bgp, aeT, yo, yi, UL, s, ND, bfg, asP, vT, asQ, iw, bbs, dq, Pp, Lc, Hu, Z, t, clV, zZ, hq, ck, hL, KZ, m, Ux, UF, Po); #c1(NR5A1) c2(XP_011516758) c3 (steroidogenic factor 1 isoform X3) c4( ) c5(310213, 310214, 310215, 310216, 310217, 310218) c6( ) c7(bL, T, gU, bbn, adW, n, aj, bS, v, ct, Ns, ci, eA, J, er, dP, PW, clU, aX, vJ, il, PD, vH, UD, iv, blo, hk, Fg, bgp, aeT, yo, yi, UL, s, ND, bfg, asP, vT, asQ, iw, bbs, dq, Pp, Lc, Hu, Z, t, clV, zZ, hq, ck, hL, KZ, m, Ux, UF, Po); #c1(NR5A2) c2(NP_001263393) c3(nuclear receptor subfamily 5 group A member 2 isoform 3) c4( ) c5(97234, 97235, 97236, 97237, 97238, 97239, 97240, 97241, 97242, 97243, 97244, 97245) c6( ) c7(T, aC, aA, bB, hX, aE, UG, aT, K, KV, bA, Qe, xQ, aiB, iU, qt, aW, fe, iV, cm, ck, iT, bE, eD, hL, im); #c1(NR5A2) c2(NP_003813) c3(nuclear receptor subfamily 5 group A member 2 isoform 2) c4(141068, 141069, 141070, 141071) c5(141072, 141073, 141074, 141075, 141076, 141077, 141078, 141079) c6( ) c7(T, aC, aA, bB, hX, aE, UG, aT, K, KV, bA, Qe, xQ, aiB, iU, qt, aW, fe, iV, cm, ck, iT, bE, eD, hL, im); #c1(NR5A2) c2(NP_995582) c3(nuclear receptor subfamily 5 group A member 2 isoform 1) c4(263926, 263927, 263928) c5(263929, 263930, 263931, 263932, 263933, 263934, 263935, 263936, 263937, 263938, 263939, 263940, 263941, 263942, 263943, 263944, 263945, 263946, 263947, 263948, 263949, 263950, 263951, 263952, 263953, 263954, 263955, 263956, 263957) c6( ) c7(T, aC, aA, bB, hX, aE, UG, aT, K, KV, bA, Qe, xQ, aiB, iU, qt, aW, fe, iV, cm, ck, iT, bE, eD, hL, im); #c1(NR5A2) c2(XP_011507686) c3(nuclear receptor subfamily 5 group A member 2 isoform X2) c4(297697) c5( ) c6( ) c7(T, aC, aA, bB, hX, aE, UG, aT, K, KV, bA, Qe, xQ, aiB, iU, qt, aW, fe, iV, cm, ck, iT, bE, eD, hL, im); #c1(NR6A1) c2(NP_001265475) c3(nuclear receptor subfamily 6 group A member 1 isoform 4) c4(98628) c5(98629, 98630, 98631, 98632, 98633, 98634) c6( ) c7(Na, T, wu, aW, eD, ff, aT, ks, rO, Nc, wm, en, nJ, ae, ad); #c1(NRGA1) c2(NP_001480) c3(nuclear receptor subfamily 6 group A member 1 isoform 2) c4(118048) c5(118049, 118050, 118051, 118052, 118053, 118054, 118055) c6( ) c7(Na, T, wu, aW, eD, ff, aT, ks, rO, Nc, wm, en, nJ, ae, ad); #c1(NRGA1) c2(NP_201591) c3(nuclear receptor subfamily 6 group A member 1 isoform 1) c4(228308) c5(228309, 228310, 228311, 228312, 228313, 228314) c6( ) c7(Na, T, wu, aW, eD, ff, aT, ks, rO, Nc, wm, en, nJ, ae, ad); #c1(NRAP) c2(NP_006166) c3(nebulin-related-anchoring protein isoform 1) c4(162473, 162474) c5(162475, 162476, 162477, 162478, 162479, 162480, 162481, 162482, 162483, 162484, 162485, 162486) c6( ) c7(vc, cc, abV); #c1(NRAP) c2(NP_932326) c3(nebulin-related-anchoring protein isoform 2) c4(258945, 258946) c5(258947, 258948, 258949, 258950, 258951, 258952, 258953, 258954, 258955, 258956, 258957, 258958) c6( ) c7(vc, cc, abV); #c1(NRAP) c2(XP_005269922) c3(nebulin-related-anchoring protein isoform X3) c4(283007) c5( ) c6( ) c7(vc, cc, abV); #c1(NRAP) c2(XP_011538134) c3(nebulin-related-anchoring protein isoform X4) c4( ) c5(335713, 335714, 335715, 335716, 335717, 335718) c6( ) c7(vc, cc, abV); #c1(NRARP) c2(NP_001004354) c3(notch-regulated ankyrin repeat-containing protein) c4(20612) c5(20613, 20614, 20615, 20616, 20617, 20618) c6( ) c7(dB); #c1(NRBP1) c2(XP_005264331) c3(nuclear receptor-binding protein isoform X1) c4(278804, 278805, 278806, 278807) c5(278808, 278809, 278810, 278811, 278812, 278813, 278814, 278815, 278816, 278817, 278818, 278819, 278820, 278821, 278822, 278823) c6( ) c7(T, ae, ad, iT); #c1(NRBP1) c2(XP_006712062) c3(nuclear receptor-binding protein isoform X2) c4( ) c5(286373, 286374, 286375, 286376, 286377, 286378, 286379, 286380, 286381, 286382, 286383, 286384) c6( ) c7(T, ae, ad, iT); #c1(NRBP2) c2(NP_848659) c3(nuclear receptor-binding protein 2) c4(254111, 254112) c5(254113, 254114, 254115, 254116, 254117, 254118, 254119, 254/20, 254121, 254122, 254123, 254124) c6( ) c7(asl); #c1(NRCAM) c2(NP_001032209) c3(neuronal cell adhesion molecule isoform A precursor) c4(31093) c5(31094, 31095, 31096, 31097, 31098, 31099, 31100, 31101, 31102, 31103) c6( ) c7(bN, ul, aRp, aQE, s, nB, lv, aW, Y, bE, X, aT, r, bB, bS, eA, nA, he, lB, q, FT); #c1(NRCAM) c2(NP_001180511) c3(neuronal cell adhesion molecule isoform D precursor) c4(74193) c5(74194, 74195, 74196, 74197, 74198, 74199) c6( ) c7(bN, ul, aRp, aQE, s, nB, lv, aW, Y, bE, X, aT, r, bB, bS, eA, nA, he, lB, q, FT); #c1(NRCAM) c2(NP_001180512) c3(neuronal cell adhesion molecule isoform E precursor) c4(74200) c5(74201, 74202, 74203, 74204, 74205, 74206) c6( ) c7(bN, ul, aRp, aDE, s, nB, lv, aW, Y, bE, X, aT, r, bB, bS, eA, nA, he, lB, q, FT); #c1(NRCAM) c2(NP_001180513) c3(neuronal cell adhesion molecule isoform F precursor) c4(74207, 74208) c5(74209, 74210, 74211, 74212, 74213, 74214, 74215, 74216, 74217, 74218, 74219) c6( ) c7(bN, ul, aRp, aQE, s, nB, lv, aW, Y, bE, X, aT, r, bB, bS, eA, nA, he, lB, q, FT); #c1(NRCAM) c2(NP_005001) c3(neuronal cell adhesion molecule isoform B precursor) c4(151942) c5(151943, 151944, 151945, 151946, 151947, 151948, 151949, 151950, 151951, 151952, 151953, 151954, 151955, 151956, 151957, 151958, 151959, 151960, 151961, 151962, 151963, 151964) c6( ) c7(bN, ul, aRp, aQE, s, nB, lv, aW, Y, bE, X, aT, r, bB, bS, eA, nA, he, lB, q, FT); #c1(NRCAM) c2(XP_005250437) c3(neuronal cell adhesion molecule isoform X14) c4( ) c5(270307) c6( ) c7(bN, ul, aRp, aQE, s, nB, lv, aW, Y, bE, X, aT, r, bB, bS, eA, nA, he, lB, q, FT); #c1(NRCAM) c2(XP_006716066) c3(neuronal cell adhesion molecule isoform X3) c4(290017, 290018) c5(290019, 290020, 290021, 290022, 290023, 290024) c6( ) c7(bN, ul, aRp, aQE, s, nB, lv, aW, Y, bE, X, aT, r, bB, bS, eA, nA, he, lB, q, FT); #c1(NRCAM) c2(XP_011514561) c3(neuronal cell adhesion molecule isoform X6) c4(307254, 307255) c5( ) c6( ) c7(bN, ul, aRp, aQE, s, nB, lv, aW, Y, bE, X, aT, r, bB, bS, eA, nA, he, lB, q, FT); #c1(NRCAM) c2(XP_011514563) c3(neuronal cell adhesion molecule isoform X9) c4(307256, 307257) c5( ) c6( ) c7(bN, ul, aRp, aQE, s, nB, lv, aW, Y, bE, X, aT, r, bB, bS, eA, nA, he, lB, q, FT); #c1(NRCAM) c2(XP_011514567) c3(neuronal cell adhesion molecule isoform X15) c4( ) c5(307258, 307259, 307260, 307261, 307262, 307263) c6( ) c7(bN, ul, aRp, aQE, s, nB, lv, aW, Y, bE, X, aT, r, bB, bS, eA, nA, he, lB, q, FT); #c1(NRCAM) c2(XP_011514573) c3(neuronal cell adhesion molecule isoform X25) c4(307264) c5(307265, 307266, 307267, 307268, 307269, 307270) c6( ) c7(bN, ul, aRp, aQE, s, nB, lv, aW, Y, bE, X, aT, r, bB, bS, eA, nA, he, lB, q, FT); #c1(NRCAM) c2(XP_011514574) c3(neuronal cell adhesion molecule isoform X26) c4( ) c5(307271, 307272, 307273, 307274, 307275, 307276, 307277, 307278, 307279, 307280, 307281, 307282) c6( ) c7(bN, ul, aRp, aQE, s, nB, lv, aW, Y, bE, X, aT, r, bB, bS, eA, nA, he, lB, q, FT); #c1(NRD1) c2(XP_011539823) c3(nardilysin isoform X1) c4(338054, 338055) c5( ) c6( ) c7(aW, ht, aT, cm, ci, hu, AO, it, af, ad, L); #c1(NRD1) c2(XP_011539824) c3(nardilysin isoform X2) c4(338056) c5( ) c6( ) c7(aW, ht, aT, cm, ci, hu, AO, it, af, ad, L); #c1(NRD1) c2(XP_011539826) c3(nardilysin isoform X4) c4( ) c5(338057, 338058, 338059, 338060, 338061) c6( ) c7(aW, ht, aT, cm, ci, hu, AO, it, af, ad, L); #c1(NRDE2) c2(NP_060440) c3(protein NRDE2 homolog) c4(195947, 195948) c5(195949, 195950, 195951, 195952, 195953, 195954, 195955, 195956, 195957, 195958, 195959, 195960) c6( ) c7(n, Y, bM); #c1(NREP) c2(NP_001135947) c3(neuronal regeneration-related protein isoform c) c4(53684, 53685) c5(53686, 53687, 53688, 53689, 53690, 53691) c6( ) c7(dE, eA, y, cz); #c1(NREP) c2(NP_001135955) c3(neuronal regeneration-related protein isoform a) c4(53692, 53693) c5(53694, 53695, 53696, 53697, 53698, 53699, 53700, 53701, 53702, 53703, 53704, 53705, 53706, 53707, 53708, 53709, 53710, 53711, 53712, 53713, 53714, 53715, 53716, 53717, 53718, 53719, 53720, 53721, 53722, 53723, 53724, 53725, 53726, 53727, 53728, 53729, 53730, 53731, 53732, 53733, 53734, 53735, 53736, 53737, 53738, 53739, 53740, 53741, 53742, 53743, 53744, 53745, 53746) c6( ) c7(dE, eA, y, cz); #c1(NREP) c2(XP_006714795) c3(neuronal regeneration-related protein isoform X1) c4( ) c5(288175) c6( ) c7(dE, eA, y, cz); #c1(NREP) c2(XP_006714796) c3(neuronal regeneration-related protein isoform X2) c4( ) c5(288176) c6( ) c7(dE, eA, y, cz); #c1(NRF1) c2(NP_001280092) c3(nuclear respiratory factor 1 isoform 2) c4(111279, 111280) c5(111281, 111282, 111283, 111284, 111285, 111286, 111287, 111288, 111289, 111290) c6( ) c7(bL, T, af, aW, eu, aT, Mz, aq, aj, bl, m, K, Ku, cm, ae, cd, J, ad, hX); #c1(NRF1) c2(NP_001280093) c3(nuclear respiratory factor 1 isoform 3) c4( ) c5(111291, 111292, 111293, 111294, 111295) c6( ) c7(bL, T, af, aW, eu, aT, Mz, aq, aj, bl, m, K, Ku, cm, ae, cd, J, ad, hX); #c1(NRF1) c2(NP_005002) c3(nuclear respiratory factor 1 isoform 1) c4(151965, 151966, 151967) c5(151968, 151969, 151970, 151971, 151972, 151973, 151974, 151975, 151976, 151977, 151978, 151979, 151980, 151981, 151982, 151983, 151984, 151985, 151986, 151987, 151988, 151989, 151990, 151991, 151992, 151993, 151994) c6( ) c7(bL, T, af, aW, eu, aT, Mz, aq, aj, bl, m, K, Ku, cm, ae, cd, J, ad, hX); #c1(NRG1) c2(NP_001153467) c3(pro-neuregulin-1, membrane-bound isoform isoform HRG-beta1c) c4(60568) c5(60569) c6( ) c7(bL, bM, T, dD, aC, ya, lT, hU, aw, ha, EQ, fZ, bO, oR, gf, j, ci, eq, V, cd, eA, amR, av, aE, pN, yt, fm, aeO, Qk, M, aT, cy, dL, bK, K, hP, an, oi, he, rz, ahE, xa, dx, af, dl, bA, eL, kl, aeR, aW, Y, Ls, AQ, ff, gl, ahl, en, Eg, Z, t, xw, bz, g, Qt, adj, jb, pH, Ew, ac, c, nB, agc, wO, eM, uR, U, tV, vL, sv, clW, uJ, m, bFC, iu); #c1(NRG1) c2(NP_001153468) c3(pro-neuregulin-1, membrane-bound isoform isoform ndf43c) c4(60570) c5(60571) c6( ) c7(bL, bM, T, dD, aC, ya, lT, hU, aw, ha, EQ, fZ, bO, oR, gf, j, ci, eq, V, cd, eA, amR, av, aE, pN, yt, fm, aeO, Qk, M, aT, cy, dL, bK, K, hP, an, oi, he, rz, ahE, xa, dx, af, dl, bA, eL, kl, aeR, aW, Y, Ls, AQ, ff, gl, ahl, en, Eg, Z, t, xw, bz, g, Qt, adj, jb, pH, Ew, ac, c, nB, agc, wO, eM, uR, U, tV, vL, sv, clW, uJ, m, bFC, iu); #c1(NRG1) c2(NP_001153471) c3(pro-neuregulin-1, membrane-bound isoform isoform HRG-beta1b) c4(60572) c5(60573) c6( ) c7(bL, bM, T, dD, aC, ya, lT, hU, aw, ha, EQ, fZ, bO, oR, gf, j, ci, eq, V, cd, eA, amR, av, aE, pN, yt, fm, aeO, Qk, M, aT, cy, dL, bK, K, hP, an, oi, he, rz, ahE, xa, dx, af, dl, bA, eL, kl, aeR, aW, Y, Ls, AQ, ff, gl, ahl, en, Eg, Z, t, xw, bz, g, Qt, adj, jb, pH, Ew, ac, c, nB, agc, wO, eM, uR, U, tV, vL, sv, clW, uJ, m, bFC, iu); #c1(NRG1) c2(NP_001153473) c3(pro-neuregulin-1, membrane-bound isoform isoform HRG-beta1d) c4(60574, 60575) c5(60576, 60577, 60578, 60579, 60580, 60581, 60582, 60583, 60584, 60585, 60586, 60587, 60588) c6( ) c7(bL, bM, T, dD, aC, ya, lT, hU, aw, ha, EQ, fZ, bO, oR, gf, j, ci, eq, V, cd, eA, amR, av, aE, pN, yt, fm, aeO, Qk, M, aT, cy, dL, bK, K, hP, an, oi, he, rz, ahE, xa, dx, af, dl, bA, eL, kl, aeR, aW, Y, Ls, AQ, ff, gl, ahl, en, Eg, Z, t, xw, bz, g, Qt, adj, jb, pH, Ew, ac, c, nB, agc, wO, eM, uR, U, tV, vL, sv, clW, uJ, m, bFC, iu); #c1(NRG1) c2(NP_001153474) c3(pro-neuregulin-1, membrane-bound isoform isoform HRG-gamma2) c4(60589) c5(60590) c6( ) c7(bL, bM, T, dD, aC, ya, lT, hU, aw, ha, EQ, fZ, bO, oR, gf, j, ci, eq, V, cd, eA, amR, av, aE, pN, yt, fm, aeO, Qk, M, aT, cy, dL, bK, K, hP, an, oi, he, rz, ahE, xa, dx, af, dl, bA, eL, kl, aeR, aW, Y, Ls, AQ, ff, gl, ahl, en, Eg, Z, t, xw, bz, g, Qt, adj, jb, pH, Ew, ac, c, nB, agc, wO, eM, uR, U, tV, vL, sv, clW, uJ, m, bFC, iu); #c1(NRG1) c2(NP_001153476) c3(pro-neuregulin-1, membrane-bound isoform isoform ndf 43b) c4(60591) c5(60592, 60593, 60594) c6( ) c7(bL, bM, T, dD, aC, ya, lT, hU, aw, ha, EQ, fZ, bO, oR, gf, j, ci, eq, V, cd, eA, amR, av, aE, pN, yt, fm, aeO, Qk, M, aT, cy, dL, bK, K, hP, an, oi, he, rz, ahE, xa, dx, af, dl, bA, eL, kl, aeR, aW, Y, Ls, AQ, ff, gl, ahl, en, Eg, Z, t, xw, bz, g, Qt, adj, jb, pH, Ew, ac, c, nB, agc, wO, eM, uR, U, tV, vL, sv, clW, uJ, m, bFC, iu); #c1(NRG1) c2(NP_001153477) c3(pro-neuregulin-1, membrane-bound isoform isoform HRG-beta3b) c4(60595) c5(60596) c6( ) c7(bL, bM, T, dD, aC, ya, lT, hU, aw, ha, EQ, fZ, bO, oR, gf, j, ci, eq, V, cd, eA, amR, av, aE, pN, yt, fm, aeO, Qk, M, aT, cy, dL, bK, K, hP, an, oi, he, rz, ahE, xa, dx, af, dl, bA, eL, kl, aeR, aW, Y, Ls, AQ, ff, gl, ahl, en, Eg, Z, t, xw, bz, g, Qt, adj, jb, pH, Ew, ac, c, nB, agc, wO, eM, uR, U, tV, vL, sv, clW, uJ, m, bFC, iu); #c1(NRG1) c2(NP_001153479) c3 (pro-neuregulin-1, membrane-bound isoform isoform HRG-gamma3) c4(60597) c5(60598) c6( ) c7(bL, bM, T, dD, aC, ya, lT, hU, aw, ha, EQ, fZ, bO, oR, gf, j, ci, eq, V, cd, eA, amR, av, aE, pN, yt, fm, aeO, Qk, M, aT, cy, dL, bK, K, hP, an, oi, he, rz, ahE, xa, dx, af, dl, bA, eL, kl, aeR, aW, Y, Ls, AQ, ff, gl, ahl, en, Eg, Z, t, xw, bz, g, Qt, adj, jb, pH, Ew, ac, c, nB, agc, wO, eM, uR, U, tV, vL, sv, clW, uJ, m, bFC, iu); #c1(NRG1) c2(NP_001153480) c3(pro-neuregulin-1, membrane-bound isoform isoform HRG-beta2b) c4( ) c5(60599, 60600, 60601, 60602, 60603) c6( ) c7(bL, bM, T, dD, aC, ya, lT, hU, aw, ha, EQ, fZ, bO, oR, gf, j, ci, eq, V, cd, eA, amR, av, aE, pN, yt, fm, aeO, Qk, M, aT, cy, dL, bK, K, hP, an, oi, he, rz, ahE, xa, dx, af, dl, bA, eL, kl, aeR, aW, Y, Ls, AQ, ff, gl, ahl, en, Eg, Z, t, xw, bz, g, Qt, adj, jb, pH, Ew, ac, c, nB, agc, wO, eM, uR, U, tV, vL, sv, clW, uJ, m, bFC, iu); #c1(NRG1) c2(NP_004486) c3(pro-neuregulin-1, membrane-bound isoform isoform HRG-gamma) c4( ) c5(146838) c6( ) c7(bL, bM, T, dD, aC, ya, lT, hU, aw, ha, EQ, fZ, bO, oR, gf, j, ci, eq, V, cd, eA, amR, av, aE, pN, yt, fm, aeO, Qk, M, aT, cy, dL, bK, K, hP, an, oi, he, rz, ahE, xa, dx, af, dl, bA, eL, kl, aeR, aW, Y, Ls, AQ, ff, gl, ahl, en, Eg, Z, t, xw, bz, g, Qt, adj, jb, pH, Ew, ac, c, nB, agc, wO, eM, uR, U, tV, vL, sv, clW, uJ, m, bFC, iu); #c1(NRG1) c2(NP_039250) c3(pro-neuregulin-1, membrane-bound isoform isoform HRG-beta1) c4(176951) c5(176952, 176953, 176954, 176955, 176956, 176957, 176958) c6( ) c7(bL, bM, T, dD, aC, ya, lT, hU, aw, ha, EQ, fZ, bO, oR, gf, j, ci, eq, V, cd, eA, amR, av, aE, pN, yt, fm, aeO, Qk, M, aT, cy, dL, bK, K, hP, an, oi, he, rz, ahE, xa, dx, af, dl, bA, eL, kl, aeR, aW, Y, Ls, AQ, ff, gl, ahl, en, Eg, Z, t, xw, bz, g, Qt, adj, jb, pH, Ew, ac, c, nB, agc, wO, eM, uR, U, tV, vL, sv, clW, uJ, m, bFC, iu); #c1(NRG1) c2(NP_039251) c3(pro-neuregulin-1, membrane-bound isoform isoform HRG-beta2) c4(176959, 176960, 176961, 176962) c5(176963, 176964, 176965, 176966, 176967, 176968, 176969, 176970, 176971, 176972, 176973, 176974, 176975, 176976, 176977, 176978, 176979, 176980, 176981, 176982, 176983, 176984, 176985, 176986, 176987, 176988, 176989, 176990, 176991, 176992, 176993, 176994, 176995, 176996, 176997, 176998, 176999, 177000, 177001, 177002, 177003, 177004, 177005, 177006, 177007, 177008, 177009, 177010, 177011, 177012, 177013, 177014, 177015, 177016, 177017, 177018, 177019, 177020, 177021, 177022, 177023, 177024, 177025, 177026, 177027, 177028, 177029, 177030, 177031, 177032, 177033, 177034, 177035, 177036, 177037, 177038, 177039, 177040, 177041, 177042, 177043, 177044, 177045, 177046, 177047, 177048, 177049, 177050, 177051, 177052, 177053, 177054, 177055, 177056, 177057, 177058, 177059, 177060, 177061, 177062, 177063, 177064) c6(177065) c7(bL, bM, T, dD, aC, ya, lT, hU, aw, ha, EQ, fZ, bO, oR, gf, j, ci, eq, V, cd, eA, amR, av, aE, pN, yt, fm, aeO, Qk, M, aT, cy, dL, bK, K, hP, an, oi, he, rz, ahE, xa, dx, af, dl, bA, eL, kl, aeR, aW, Y, Ls, AQ, ff, gl, ahl, en, Eg, Z, t, xw, bz, g, Qt, adj, jb, pH, Ew, ac, c, nB, agc, wO, eM, uR, U, tV, vL, sv, clW, uJ, m, bFC, iu); #c1(NRG1) c2(NP_039252) c3(pro-neuregulin-1, membrane-bound isoform isoform HRG-beta3) c4(177066) c5(177067) c6( ) c7(bL, bM, T, dD, aC, ya, lT, hU, aw, ha, EQ, fZ, bO, oR, gf, j, ci, eq, V, cd, eA, amR, av, aE, pN, yt, fm, aeO, Qk, M, aT, cy, dL, bK, K, hP, an, oi, he, rz, ahE, xa, dx, af, dl, bA, eL, kl, aeR, aW, Y, Ls, AQ, ff, gl, ahl, en, Eg, Z, t, xw, bz, g, Qt, adj, jb, pH, Ew, ac, c, nB, agc, wO, eM, uR, U, tV, vL, sv, clW, uJ, m, bFC, iu); #c1(NRG1) c2(NP_039253) c3(pro-neuregulin-1, membrane-bound isoform isoform SMDF) c4(177068) c5(177069, 177070, 177071, 177072, 177073, 177074, 177075) c6( ) c7(bL, bM, T, dD, aC, ya, lT, hU, aw, ha, EQ, fZ, bO, oR, gf, j, ci, eq, V, cd, eA, amR, av, aE, pN, yt, fm, aeO, Qk, M, aT, cy, dL, bK, K, hP, an, oi, he, rz, ahE, xa, dx, af, dl, bA, eL, kl, aeR, aW, Y, Ls, AQ, ff, gl, ahl, en, Eg, Z, t, xw, bz, g, Qt, adj, jb, pH, Ew, ac, c, nB, agc, wO, eM, uR, U, tV, vL, sv, clW, uJ, m, bFC, iu); #c1(NRG1) c2(NP_039254) c3(pro-neuregulin-1, membrane-bound isoform isoform ndf 43) c4(177076) c5(177077, 177078, 177079, 177080, 177081, 177082, 177083, 177084, 177085, 177086, 177087, 177088) c6( ) c7(bL, bM, T, dD, aC, ya, lT, hU, aw, ha, EQ, fZ, bO, oR, gf, j, ci, eq, V, cd, eA, amR, av, aE, pN, yt, fm, aeO, Qk, M, aT, cy, dL, bK, K, hP, an, oi, he, rz, ahE, xa, dx, af, dl, bA, eL, kl, aeR, aW, Y, Ls, AQ, ff, gl, ahl, en, Eg, Z, t, xw, bz, g, Qt, adj, jb, pH, Ew, ac, c, nB, agc, wO, eM, uR, U, tV, vL, sv, clW, uJ, m, bFC, iu); #c1(NRG1) c2(NP_039256) c3(pro-neuregulin-1, membrane-bound isoform isoform GGF2 precursor) c4(177089) c5(177090, 177091, 177092, 177093, 177094, 177095, 177096, 177097, 177098, 177099, 177100, 177101, 177102, 177103, 177104, 177105, 177106, 177107, 177108, 177109, 177110, 177111, 177112, 177113, 177114, 177115, 177116, 177117, 177118, 177119, 177120, 177121, 177122, 177123, 177124, 177125, 177126, 177127, 177128, 177129, 177130, 177131, 177132, 177133, 177134, 177135, 177136, 177137, 177138, 177139, 177140, 177141, 177142, 177143, 177144, 177145, 177146, 177147, 177148, 177149, 177150, 177151, 177152, 177153, 177154, 177155, 177156, 177157, 177158, 177159, 177160, 177161, 177162, 177163, 177164, 177165, 177166, 177167, 177168, 177169, 177170, 177171, 177172, 177173, 177174, 177175, 177176, 177177, 177178, 177179, 177180, 177181, 177182) c6( ) c7(bL, bM, T, dD, aC, ya, lT, hU, aw, ha, EQ, fZ, bO, oR, gf, j, ci, eq, V, cd, eA, amR, av, aE, pN, yt, fm, aeO, Qk, M, aT, cy, dL, bK, K, hP, an, oi, he, rz, ahE, xa, dx, af, dl, bA, eL, kl, aeR, aW, Y, Ls, AQ, ff, gl, ahl, en, Eg, Z, t, xw, bz, g, Qt, adj, jb, pH, Ew, ac, c, nB, agc, wO, eM, uR, U, tV, vL, sv, clW, uJ, m, bFC, iu); #c1(NRG1) c2(NP_039258) c3(pro-neuregulin-1, membrane-bound isoform isoform HRG-alpha) c4(177183, 177184, 177185) c5(177186, 177187, 177188, 177189, 177190, 177191, 177192, 177193, 177194, 177195, 177196, 177197, 177198, 177199, 177200, (77201, 177202) c6( ) c7(bL, bM, T, dD, aC, ya, lT, hU, aw, ha, EQ, fZ, bO, oR, gf, j, ci, eq, V, cd, eA, amR, av, aE, pN, yt, fm, aeO, Qk, M, aT, cy, dL, bK, K, hP, an, oi, he, rz, ahE, xa, dx, af, dl, bA, eL, kl, aeR, aW, Y, Ls, AQ, ff, gl, ahl, en, Eg, Z, t, xw, bz, g, Qt, adj, jb, pH, Ew, ac, c, nB, agc, wO, eM, uR, U, tV, vL, sv, clW, uJ, m, bFC, iu); #c1(NRG2) c2(NP_001171864) c3(pro-neuregulin-2, membrane-bound isoform isoform 7) c4(71951) c5(71952, 71953, 71954, 71955, 71956, 71957) c6( ) c7(aW, ac, aT, pZ, bU); #c1(NRG2) c2(NP_004874) c3(pro-neuregulin-2, membrane-bound isoform isoform 1) c4(150459) c5(150460, 150461, 150462, 150463, 150464, 150465) c6( ) c7(aW, ac, aT, pZ, bU); #c1(NRG2) c2(NP_053584) c3(pro-neuregulin-2, membrane-bound isoform isoform 2) c4(177237) c5(177238, 177239, 177240, (77241, 177242, 177243) c6( ) c7(aW, ac, aT, pZ, bU); #c1(NRG2) c2(NP_053585) c3(pro-neuregulin-2, membrane-bound isoform isoform 3) c4(177244, 177245) c5(177246, 177247, 177248, 177249, 177250, 177251, 177252, 177253, 177254, 177255, 177256, 177257) c6( ) c7(aW, ac, aT, pZ, bU); #c1(NRG2) c2(NP_053586) c3(pro-neuregulin-2, membrane-bound isoform isoform 4) c4(177258) c5(177259, 177260, 177261, 177262, 177263, 177264) c6( ) c7(aW, ac, aT, pZ, bU); #c1(NRG2) c2(XP_006714873) c3(pro-neuregulin-2, membrane-bound isoform isoform X7) c4(288288, 288289) c5(288290, 288291, 288292, 288293, 288294, 288295, 288296, 288297, 288298, 288299, 288300, 288301) c6( ) c7(aW, ac, aT, pZ, bU); #c1(NRG3) c2(NP_001010848) c3(pro-neuregulin-3, membrane-bound isoform isoform 1) c4(23831) c5(23832, 23833, 23834, 23835, 23836, 23837) c6( ) c7(bM, aC, af, rz, yW, aW, Fo, oe, aw, aT, cz, xg, akY, bK, ahl, Eg, he, ahE, cd); #c1(NRG3) c2(NP_001159444) c3(pro-neuregulin-3, membrane-bound isoform isoform 2) c4(64068, 64069) c5(64070, 64071, 64072, 64073, 64074, 64075, 64076) c6( ) c7(bM, aC, af, rz, yW, aW, Fo, oe, aw, aT, cz, xg, akY, bK, ahl, Eg, he, ahE, cd); #c1(NRG3) c2(NP_001159445) c3 (pro-neuregulin-3, membrane-bound isoform isoform 3) c4(64077) c5(64078, 64079, 64080, 64081, 64082, 64083) c6( ) c7(bM, aC, af, rz, yW, aW, Fo, oe, aw, aT, cz, xg, akY, bK, ahl, Eg, he, ahE, cd); #c1(NRG3) c2(XP_005269501) c3(pro-neuregulin-3, membrane-bound isoform isoform X2) c4(282605) c5(282606, 282607, 282608, 282609, 282610, 282611) c6( ) c7(bM, aC, af, rz, yW, aW, Fo, oe, aw, aT, cz, xg, akY, bK, ahl, Eg, he, ahE, cd); #c1(NRG3) c2(XP_011537479) c3(pro-neuregulin-3, membrane-bound isoform isoform X7) c4( ) c5(335087, 335088, 335089, 335090, 335091, 335092, 335093, 335094, 335095, 335096) c6( ) c7(bM, aC, af, rz, yW, aW, Fo, oe, aw, aT, cz, xg, akY, bK, ahl, Eg, he, ahE, cd); #c1(NRG4) c2(NP_612640) c3(pro-neuregulin-4, membrane-bound isoform) c4(236737, 236738, 236739) c5(236740, 236741, 236742, 236743, 236744, 236745, 236746, 236747, 236748, 236749, 236750, 236751, 236752, 236753, 236754, 236755, 236756, 236757) c6( ) c7(bL, RT, lR, fc, aqV, fe, nw, dL, v, cP, ji, nC, ae, ad); #c1(NRGN) c2(NP_001119653) c3(neurogranin) c4(44282) c5(44283, 44284, 44285, 44286, 44287, 44288, 44289, 44290, 44291, 44292, 44293, 44294, 44295, 44296) c6( ) c7(gl, bK, K, bC, yA, cm, ae, eA, ad); #c1(NRIP1) c2(XP_005261122) c3(nuclear receptor-interacting protein 1 isoform X1) c4(277220, 277221) c5(277222, 277223, 277224, 277225, 277226, 277227, 277228, 277229, 277230, 277231, 277232, 277233, 277234, 277235, 277236, 277237, 277238, 277239, 277240, 277241) c6( ) c7(T, gU, aw, bB, uu, cd, dP, aT, bA, JU, vT, aW, fc, dq, g, iV, t, fq, yE, bE, eD, gZ, m, cP, y); #c1(NRIP2) c2(NP_113662) c3(nuclear receptor-interacting protein 2) c4(221996, 221997) c5(221998, 221999, 222000, 222001, 222002, 222003, 222004, 222005, 222006, 222007, 222008, 222009) c6( ) c7(j); #c1(NRK) c2(NP_940867) c3(nik-related protein kinase) c4(259991) c5(259992, 259993, 259994, 259995, 259996, 259997, 259998, 259999, 260000, 260001, 260002, 260003, 260004, 260005, 260006, 260007, 260008) c6( ) c7(ck, je, iM, kH); #c1(NRK) c2(XP_006724695) c3(nik-related protein kinase isoform X1) c4(296261, 296262, 296263) c5( ) c6( ) c7(ck, je, iM, kH); #c1(NRK) c2(XP_006724697) c3(nik-related protein kinase isoform X4) c4( ) c5(296264, 296265, 296266, 296267, 296268, 296269) c6( ) c7(ck, je, iM, kH); #c1(NRK) c2(XP_011529189) c3(nik-related protein kinase isoform X3) c4( ) c5(324592) c6( ) c7(ck, je, iM, KH); #c1(NRL) c2(XP_011535103) c3(neural retina-specific leucine zipper protein isoform X2) c4(331893, 331894, 331895) c5(331896, 331897, 331898, 331899) c6( ) c7(clX, clZ, xx, lR, alF, bFo, nx, clY, nw, nk, Bd, nC, ayj); #c1(NRL) c2(XP_011535107) c3(neural retina-specific leucine zipper protein isoform X1) c4( ) c5(331900, 331901, 331902, 331903, 331904, 331905, 331906, 331907, 331908, 331909, 331910, 331911, 331912, 331913, 331914, 331915) c6( ) c7(clX, clZ, xx, lR, alF, bFo, nx, clY, nw, nk, Bd, nC, ayj); #c1(NRL) c2(XP_011535108) c3(neural retina-specific leucine zipper protein isoform X3) c4( ) c5(331916, 331917, 331918, 331919, 331920, 331921, 331922, 331923, 331924, 331925) c6( ) c7(clX, clZ, xx, lR, alF, bFo, nx, clY, nw, nk, Bd, nC, ayj); #c1(NRM) c2(NP_001257638) c3(nurim isoform 4) c4(95524) c5(95525, 95526, 95527, 95528, 95529, 95530, 95531, 95532, 95533, 95534, 95535, 95536, 95537, 95538, 95539, 95540, 95541, 95542, 95543, 95544, 95545, 95546, 95547, 95548) c6( ) c7(y, eZ, cj); #c1(NRM) c2(NP_009174) c3(nurim isoform 1) c4(171256, 171257) c5(171258, 171259, 171260, 171261, 171262, 171263, 171264, 171265, 171266, 171267, 171268, 171269, 171270, 171271, 171272, 171273, 171274, 171275, 171276, 171277, 171278, 171279, 171280, 171281, 171282, 171283, 171284, 171285, 171286, 171287, 171288, 171289, 171290, 171291, 171292, 171293, 171294, 171295, 171296, 171297, 171298, 171299, 171300, 171301, 171302, 171303, 171304, 171305, 171306, 171307, 171308, 171309) c6( ) c7(y, eZ, cj); #c1(NRN1) c2(NP_001265639) c3(neuritin isoform 1 precursor) c4(99076) c5(99077, 99078, 99079, 99080, 99081, 99082) c6( ) c7(KC, ke, T, xg, ek); #c1(NRP1) c2(NP_001019799) c3(neuropilin-1 isoform b precursor) c4(27841, 27842, 27843) c5(27844, 27845, 27846, 27847, 27848, 27849, 27850, 27851, 27852, 27853, 27854, 27855, 27856, 27857, 27858, 27859, 27860, 27861) c6( ) c7(T, aC, bVx, ea, bO, bB, ci, x, aE, pX, K, eL, aiJ, ago, aW, Ls, hl, g, as, cm, eM, iT, acw, vy, cj, k, m, asW, dD, sw, aw, ht, NT, fr, ct, qN, Vs, yn, ft, gd, Ne, h, hn, dL, vx, aze, hi, A, ack, aq, dB, ayS, YH, aPG, B, aA, bX, q, eA, eJ, bza, aT, cy, aPF, ae, Y, ev, r, en, t, ck, Ta, sS, iM, dy, EY, UF, bt, ao, hS, HQ, jh, bS, aFR, eq, hF, gb, ad, dP, av, hd, fX, Cj, yt, fb, d, je, ff, af, Z, bz, ek, VO, bE, oQ, y, vt, ne, Tc); #c1(NRP1) c2(NP_001019800) c3 (neuropilin-1 isoform c precursor) c4(27862) c5(27863, 27864, 27865, 27866, 27867, 27868, 27869, 27870, 27871, 27872, 27873, 27874, 27875, 27876, 27877, 27878, 27879, 27880, 27881, 27882, 27883, 27884, 27885) c6( ) c7(T, aC, bVx, ea, bO, bB, ci, x, aE, pX, K, eL, aiJ, ago, aW, Ls, hl, g, as, cm, eM, iT, acw, vy, cj, k, m, asW, dD, sw, aw, ht, NT, fr, ct, qN, Vs, yn, ft, gd, Ne, h, hn, dl, vx, aze, hi, A, ack, aq, dB, ayS, YH, aPG, B, aA, bX, q, eA, eJ, bza, aT, cy, aPF, ae, Y, ev, r, en, t, ck, Ta, sS, iM, dy, EY, UF, bt, ao, hS, HQ, jh, bS, aFR, eq, hF, gb, ad, dP, av, hd, fX, Cj, yt, fb, d, je, ff, aF, Z, bz, ek, VO, bE, oQ, y, vt, ne, Tc); #c1(NRP1) c2(NP_001231901) c3(neuropilin-1 isoform d precursor) c4(87678) c5( ) c6( ) c7(T, aC, bVx, ea, bO, bB, ci, x, aE, pX, K, eL, aiJ, ago, aW, Ls, hl, g, as, cm, eM, iT, acw, vy, cj, k, m, asW, dD, sw, aw, ht, NT, fr, ct, qN, Vs, yn, ft, gd, Ne, h, hn, dL, vx, aze, hi, A, ack, aq, dB, ayS, YH, aPG, B, aA, bX, q, eA, eJ, bza, aT, cy, aPF, ae, Y, ev, r, en, t, ck, Ta, sS, iM, dy, EY, UF, bt, ao, hS, HQ, jh, bS, aFR, eq, hF, gb, ad, dP, av, hd, fX, Cj, yt, fb, d, je, ff, af, Z, bz, ek, VO, bE, oQ, y, vt, ne, Tc); #c1 (NRP1) c2(NP_001231902) c3(neuropilin-1 isoform e precursor) c4(87679, 87680) c5(87681, 87682, 87683, 87684, 87685, 87686) c6( ) c7(T, aC, bVx, ea, bO, bB, ci, x, aE, pX, K, eL, aiJ, ago, aW, Ls, hl, g, as, cm, eM, iT, acw, vy, cj, k, m, asW, dD, sw, aw, ht, NT, fr, ct, qN, Vs, yn, ft, gd, Ne, h, hn, dL, vx, aze, hi, A, ack, aq, dB, ayS, YH, aPG, B, aA, bX, q, eA, eJ, bza, aT, cy, aPF, ae, Y, ev, r, en, t, ck, Ta, sS, iM, dy, EY, UF, bt, ao, hS, HQ, jh, bS, aFR, eq, hF, gb, ad, dP, av, hd, fX, Cj, yt, fb, d, je, ff, aF, Z, bz, ek, VO, bE, oQ, y, vt, ne, Tc); #c1(NRP1) c2(NP_003864) c3(neuropilin-1 isoform a precursor) c4(141603) c5(141604, 141605, 141606, 141607, 141608, 141609, 141610, 141611, 141612, 141613) c6( ) c7(T, aC, bVx, ea, bO, bB, ci, x, aE, pX, K, eL, aiJ, ago, aW, Ls, hl, g, as, cm, eM, iT, acw, vy, cj, k, m, asW, dD, sw, aw, ht, NT, fr, ct, qN, Vs, yn, ft, gd, Ne, h, hn, dL, vx, aze, hi, A, ack, aq, dB, ayS, YH, aPG, B, aA, bX, q, eA, eJ, bza, aT, cy, aPF, ae, Y, ev, r, en, t, ck, Ta, sS, iM, dy, EY, UF, bt, ao, hS, HQ, jh, bS, aFR, eq, hF, gb, ad, dP, av, hd, fX, Cj, yt, fb, d, je, ff, af, Z, bz, ek, VO, bE, oQ, y, vt, ne, Tc); #c1(NRP1) c2(XP_006717584) c3(neuropilin-1 isoform X1) c4(291181) c5( ) c6( ) c7(T, aC, bVx, ea, bO, bB, ci, x, aE, pX, K, eL, aiJ, ago, aW, Ls, hl, g, as, cm, eM, iT, acw, vy, cj, k, m, asW, dD, sw, aw, ht, NT, fr, ct, qN, Vs, yn, ft, gd, Ne, h, hn, dL, vx, aze, hi, A, ack, aq, dB, ayS, YH, aPG, B, aA, bX, q, eA, eJ, bza, aT, cy, aPF, ae, Y, ev, r, en, t, ck, Ta, sS, iM, dy, EY, UF, bt, ao, hS, HQ, jh, bS, aFR, eq, hF, gb, ad, dP, av, hd, fX, Cj, yt, fb, d, je, ff, aF, Z, bz, ek, VO, bE, oQ, y, vt, ne, Tc); #c1(NRP1) c2(XP_006717586) c3(neuropilin-1 isoform X3) c4(291182, 291183) c5(291184, 291185, 29/186, 291187, 291188, 291189, 291190, 291191, 291192, 291193, 291194, 291195) c6( ) c7(T, aC, bVx, ea, bO, bB, ci, x, aE, pX, K, eL, aiJ, ago, aW, Ls, hl, g, as, cm, eM, iT, acw, vy, cj, k, m, asW, dD, sw, aw, ht, NT, fr, ct, qN, Vs, yn, ft, gd, Ne, h, hn, dL, vx, aze, hi, A, ack, aq, dB, ayS, YH, aPG, B, aA, bX, q, eA, eJ, bza, aT, cy, aPF, ae, Y, ev, r, en, t, ck, Ta, sS, iM, dy, EY, UF, bt, ao, hS, HQ, jh, bS, aFR, eq, hF, gb, ad, dP, av, hd, fX, Cj, yt, fb, d, je, ff, aF, Z, bz, ek, VO, bE, oQ, y, vt, ne, Tc); #c1(NRP1) c2(XP_006717587) c3(neuropilin-1 isoform X4) c4(291196) c5( ) c6( ) c7(T, aC, bVx, ea, bO, bB, ci, x, aE, pX, K, eL, aiJ, ago, aW, Ls, hl, g, as, cm, eM, iT, acw, vy, cj, k, m, asW, dD, sw, aw, ht, NT, fr, ct, qN, Vs, yn, ft, gd, Ne, h, hn, dL, vx, aze, hi, A, ack, aq, dB, ayS, YH, aPG, B, aA, bX, q, eA, eJ, bza, aT, cy, aPF, ae, Y, ev, r, en, t, ck, Ta, sS, iM, dy, EY, UF, bt, ao, hS, HQ, jh, bS, aFR, eq, hF, gb, ad, dP, av, hd, fX, Cj, yt, fb, d, je, ff, aF, Z, bz, ek, VO, bE, oQ, y, vt, ne, Tc); #c1 (NRP1) c2(XP_006717589) c3(neuropilin-1 isoform X6) c4(291197, 291198) c5(291199, 291200, 291201, 291202, 29/203, 291204, 291205, 291206, 291207, 291208, 291209, 291210, 291211, 291212, 291213, 291214, 291215, 291216) c6( ) c7(T, aC, bVx, ea, bO, bB, ci, x, aE, pX, K, eL, aiJ, ago, aW, Ls, hl, g, as, cm, eM, iT, acw, vy, cj, k, m, asW, dD, sw, aw, ht, NT, fr, ct, qN, Vs, yn, ft, gd, Ne, h, hn, dL, vx, aze, hi, A, ack, aq, dB, ayS, YH, aPG, B, aA, bX, q, eA, BJ, bza, aT, cy, aPF, ae, Y, ev, r, en, t, ck, Ta, sS, iM, dy, EY, UF, bt, ao, hS, HQ, jh, bS, aFR, eq, hF, gb, ad, dP, av, hd, fX, Cj, yt, fb, d, je, ff, aF, Z, bz, ek, VO, bE, oQ, y, vt, ne, Tc); #c1(NRP1) c2(XP_011518057) c3(neuropilin-1 isoform X7) c4(312098) c5( ) c6( ) c7(T, aC, bVx, ea, bO, bB, ci, x, aE, pX, K, eL, aiJ, ago, aW, Ls, hl, g, as, cm, eM, iT, acw, vy, cj, k, m, asW, dD, sw, aw, ht, NT, fr, ct, qN, Vs, yn, ft, gd, Ne, h, hn, dL, vx, aze, hi, A, ack, aq, dB, ayS, YH, aPG, B, aA, bX, q, eA, eJ, bza, aT, cy, aPF, ae, Y, ev, r, en, t, ck, Ta, sS, iM, dy, EY, UF, bt, ao, hS, HQ, jh, bS, aFR, eq, hF, gb, ad, dP, av, hd, fX, Cj, yt, fb, d, je, ff, aF, Z, bz, ek, VO, bE, oQ, y, vt, ne, Tc); #c1(NRP1) c2(XP_011518058) c3(neuropilin-1 isoform X8) c4( ) c5(312099, 312100, 312101, 312102, 312103, 312104, 312105) c6( ) c7(T, aC, bVx, ea, bO, bB, ci, x, aE, pX, K, eL, aiJ, ago, aW, Ls, hl, g, as, cm, eM, iT, acw, vy, cj, k, m, asW, dD, sw, aw, ht, NT, fr, ct, qN, Vs, yn, ft, gd, Ne, h, hn, dL, vx, aze, hi, A, ack, aq, dB, ayS, YH, aPG, B, aA, bX, q, eA, eJ, bza, aT, cy, aPF, ae, Y, ev, r, en, t, ck, Ta, sS, iM, dy, EY, UF, bt, ao, hS, HQ, jh, bS, aFR, eq, hF, gb, ad, dP, av, hd, fX, Cj, yt, fb, d, je, ff, aF, Z, bz, ek, VO, bE, oQ, y, vt, ne, Tc); #c1(NRP2) c2(NP_003863) c3(neuropilin-2 isoform 2 precursor) c4(141589, 141590) c5(141591, 141592, 141593, 141594, 141595, 141596, 141597, 141598, 141599, 141600, 141601, 141602) c6( ) c7(T, aC, ha, bB, v, fr, ci, Cd, Cb, buj, q, ad, eA, Jm, eJ, yn, av, py, yt, apG, aT, Vv, vw, dL, K, KV, ae, eL, jf, d, adF, aW, Y, r, vt, g, bz, bN, yR, kP, uL, iT, bE, gX, dy, EY, m, UF, bt, hS); #c1(NRP2) c2(NP_061004) c3(neuropilin-2 isoform 4 precursor) c4(198917, 198918, 198919) c5(198920, 198921, 198922, 198923, 198924, 198925) c6( ) c7(T, aC, ha, bB, v, fr, ci, Cd, Cb, buj, q, ad, eA, Jm, eJ, yn, av, py, yt, apG, aT, Vv, vw, dL, K, KV, ae, eL, jf, d, adF, aW, Y, r, vt, g, bz, bN, yR, kP, uL, iT, bE, gX, dy, EY, m, UF, bt, hS); #c1(NRP2) c2(NP_957716) c3(neuropilin-2 isoform 6 precursor) c4(262023) c5(262024, 262025, 262026, 262027, 262028, 262029, 262030, 262031, 262032, 262033, 262034, 262035, 262036, 262037, 262038, 262039, 262040, 262041, 262042, 262043, 262044, 262045) c6( ) c7(T, aC, ha, bB, v, fr, ci, Cd, Cb, buj, q, ad, eA, Jm, eJ, yn, av, py, yt, apG, aT, Vv, vw, dL, K, KV, ae, eL, jf, d, adF, aW, Y, r, vt, g, bz, bN, yR, kP, uL, iT, bE, gX, dy, EY, m, UF, bt, hS); #c1(NRP2) c2(NP_957718) c3(neuropilin-2 isoform 1 precursor) c4(262046) c5(262047, 262048, 262049, 262050, 262051, 262052) c6( ) c7(T, aC, ha, bB, v, fr, ci, Cd, Cb, buj, q, ad, eA, Jm, eJ, yn, av, py, yt, apG, aT, Vv, vw, dL, K, KV, ae, eL, jf, d, adF, aW, Y, r, vt, g, bz, bN, yR, kP, uL, iT, bE, gX, dy, EY, m, UF, bt, hS); #c1(NRP2) c2(NP_957719) c3 (neuropilin-2 isoform 5 precursor) c4(262053) c5(262054, 262055, 262056, 262057, 262058, 262059) c6( ) c7(T, aC, ha, bB, v, fr, ci, Cd, Cb, buj, q, ad, eA, Jm, eJ, yn, av, py, yt, apG, aT, Vv, vw, dL, K, KV, ae, eL, jf, d, adF, aW, Y, r, vt, g, bz, bN, yR, kP, ul, iT, bE, gX, dy, EY, m, UF, bt, hS); #c1(NRP2) c2(NP_958436) c3(neuropilin-2 isoform 3 precursor) c4(262077, 262078) c5(262079, 262080, 262081, 262082, 262083, 262084, 262085, 262086, 262087, 262088, 262089, 262090) c6( ) c7(T, aC, ha, bB, v, fr, ci, Cd, Cb, buj, q, ad, eA, Jm, eJ, yn, av, py, yt, apG, aT, Vv, vw, dL, K, KV, ae, eL, jf, d, adF, aW, Y, r, vt, g, bz, bN, yR, kP, uL, iT, bE, gX, dy, EY, m, UF, bt, hS); #c1(NRSN1) c2(NP_542454) c3(neurensin-1) c4(232997, 232998, 232999) c5(233000, 233001, 233002, 233003, 233004, 233005, 233006, 233007, 233008, 233009, 233010, 233011, 233012, 233013, 233014, 233015, 233016, 233017, 233018, 233019, 233020, 233021) c6( ) c7(if, iW, qN, hX, ds, jZ, dV, kH, h, SH, he, CB, je, aHW, Pg, lo, kk, cm, ck, iM, iQ, cA, bE, NL, aEG, zv, yA, aEN, EB); #c1 (NRSN2) c2(NP_079234) c3(neurensin-2) c4(217562) c5(217563, 217564, 217565, 217566, 217567, 217568, 217569, 217570, 217571, 217572) c6( ) c7(aaw); #c1(NRTN) c2(XP_011526343) c3(neurturin isoform X1) c4(320641) c5(320642, 320643, 320644, 320645, 320646, 320647) c6( ) c7(cma, aJf, aE, fm, Qk, Ji, an, U, bK, fu, qF, ahl, ahE, aJe); #c1(NRXN1) c2(NP_001129131) c3(neurexin-1-beta isoform alpha2 precursor) c4(50866, 50867) c5(50868, 50869, 50870, 50871, 50872, 50873, 50874, 50875, 50876, 50877, 50878, 50879) c6( ) c7(ahc, oe, ha, atS, amj, bS, ct, nA, aQK, hb, XY, cmb, lG, Ko, aQM, bK, he, rz, A, VX, yL, Az, aha, Z, bN, Cq, Kr, cmc, Wl, y); #c1(NRXN1) c2(NP_004792) c3(neurexin-1-beta isoform alphal precursor) c4(149892, 149893) c5(149894, 149895, 149896, 149897, 149898, 149899, 149900, 149901, 149902, 149903, 149904, 149905) c6( ) c7(ahc, oe, ha, atS, amj, bS, ct, nA, aQK, hb, XY, cmb, lG, Ko, aQM, bK, he, rz, A, VX, yL, Az, aha, Z, bN, Cq, Kr, cmc, Wl, y); #c1(NRXN1) c2(NP_620072) c3(neurexin-1-beta isoform beta precursor) c4(237255, 237256) c5(237257, 237258, 237259, 237260, 237261, 237262, 237263, 237264, 237265, 237266, 237267, 237268) c6( ) c7(ahc, oe, ha, atS, amj, bS, ct, nA, aQK, hb, XY, cmb, lG, Ko, aQM, bK, he, rz, A, VX, yL, Az, aha, Z, bN, Cq, Kr, cmc, Wl, y); #c1(NRXN1) c2(XP_011531473) c3 (neurexin-1-beta isoform X6) c4(327596) c5(327597, 327598, 327599, 327600, 327601, 327602) c6( ) c7(ahc, oe, ha, atS, amj, bS, ct, nA, aQK, hb, XY, cmb, lG, Ko, aQM, bk, he, rz, A, VX, yL, Az, aha, Z, bN, Cq, Kr, cmc, Wl, y); #c1(NRXN1) c2(XP_011531476) c3(neurexin-1-beta isoform X9) c4(327603) c5(327604, 327605, 327606, 327607, 327608, 327609) c6( ) c7(ahc, oe, ha, atS, amj, bS, ct, nA, aQK, hb, XY, cmb, lG, Ko, aQM, bK, he, rz, A, VX, yL, Az, aha, Z, bN, Cq, Kr, cmc, Wl, y); #c1(NRXN1) c2(XP_011531479) c3(neurexin-1-beta isoform X13) c4(327610) c5( ) c6( ) c7(ahc, oe, ha, atS, amj, bS, ct, nA, aQK, hb, XY, cmb, lG, Ko, aQM, bK, he, rz, A, VX, yL, Az, aha, Z, bN, Cq, Kr, cmc, Wl, y); #c1(NRXN1) c2(XP_011531481) c3(neurexin-1-beta isoform X16) c4( ) c5(327611, 327612, 327613, 327614, 327615, 327616) c6( ) c7(ahc, oe, ha, atS, amj, bS, ct, nA, aQK, hb, XY, cmb, lG, Ko, aQM, bK, he, rz, A, VX, yL, Az, aha, Z, bN, Cq, Kr, cmc, Wl, y); #c1(NRXN2) c2(NP_055895) c3(neurexin-2-beta isoform alpha-1 precursor) c4(184277) c5(184278, 184279, 184280, 184281, 184282, 184283, 184284, 184285, 184286, 184287) c6( ) c7(rz, Kr, VX, bfM, aT); #c1(NRXN2) c2(NP_620060) c3(neurexin-2-beta isoform alpha-2 precursor) c4(237226, 237227) c5(237228, 237229, 237230, 237231, 237232, 237233, 237234, 237235, 237236, 237237, 237238, 237239) c6( ) c7(rz, Kr, VX, bfM, aT); #c1(NRXN2) c2(NP_620063) c3(neurexin-2-beta isoform beta precursor) c4(237248) c5(237249, 237250, 237251, 237252, 237253, 237254) c6( ) c7(rz, Kr, VX, bfM, aT); #c1(NRXN2) c2(XP_005274457) c3(neurexin-2-beta isoform X11) c4(285083) c5(285084, 285085, 285086, 285087, 285088, 285089) c6( ) c7(rz, Kr, VX, bfM, aT); #c1(NRXN3) c2(NP_001098720) c3(neurexin 3 isoform 3 precursor) c4(41562) c5(41563, 41564, 41565, 41566, 41567, 41568) c6( ) c7(Na, bN, s, rz, hb, XY, cz, hD, Nc, v, j, nA, g, oi, he, lB, fq, eA, FT, hZ); #c1(NRXN3) c2(NP_001258949) c3 (neurexin 3 isoform 4 precursor) c4(96814) c5(96815, 96816, 96817, 96818, 96819, 96820) c6( ) c7(Na, bN, s, rz, hb, XY, cz, hD, Nc, v, j, nA, g, oi, he, lB, fq, eA, FT, hZ); #c1(NRXN3) c2(NP_004787) c3(neurexin 3 isoform 1) c4(149862) c5(149863, 149864, 149865, 149866, 149867, 149868, 149869, 149870) c6( ) c7(Na, bN, s, rz, hb, XY, cz, hD, Nc, v, j, nA, g, oi, he, lB, fq, eA, FT, hZ); #c1(NRXN3) c2(NP_620426) c3(neurexin 3 isoform 2 precursor) c4(237618) c5(237619, 237620, 237621, 237622, 237623, 237624) c6( ) c7(Na, bN, s, rz, hb, XY, cz, hD, Nc, v, j, nA, g, oi, he, lB, fq, eA, FT, hZ); #c1(NRXN3) c2(XP_011535669) c3(neurexin 3 isoform X5) c4(332903) c5(332904, 332905, 332906, 332907, 332908, 332909) c6( ) c7(Na, bN, s, rz, hb, XY, cz, hD, Nc, v, j, nA, g, oi, he, lB, fq, eA, FT, hZ); #c1(NRXN3) c2(XP_011535675) c3(neurexin 3 isoform X12) c4( ) c5(332910, 332911) c6( ) c7(Na, bN, s, rz, hb, XY, cz, hD, Nc, v, j, nA, g, oi, he, lB, fq, eA, FT, hZ); #c1(NRXN3) c2(XP_011535676) c3(neurexin 3 isoform X13) c4(332912) c5(332913, 332914, 332915, 332916, 332917, 332918) c6( ) c7(Na, bN, s, rz, hb, XY, cz, hD, Nc, v, j, nA, g, oi, he, lB, fq, eA, FT, hZ); #c1(NRXN3) c2(XP_011535678) c3(neurexin 3 isoform X15) c4(332919) c5( ) c6( ) c7(Na, bN, s, rz, hb, XY, cz, hD, Nc, v, j, nA, g, oi, he, lB, fq, eA, FT, hZ); #c1(NSA2) c2(NP_055701) c3(ribosome biogenesis protein NSA2 homolog isoform 1) c4(182880) c5(182881, 182882, 182883, 182884, 182885) c6( ) c7(bL, aj, dG, dy, bc, J); #c1(NSA2) c2(XP_011541400) c3(ribosome biogenesis protein NSA2 homolog isoform X1) c4( ) c5(340009) c6( ) c7(bL, aj, dG, dy, bc, J); #c1(NSD1) c2(XP_005266018) c3(histone-lysine N-methyltransferase, H3 lysine-36 and H4 lysine-20 specific isoform X2) c4(279857, 279858, 279859, 279860) c5(279861, 279862, 279863, 279864, 279865, 279866, 279867, 279868, 279869, 279870, 279871, 279872, 279873, 279874, 279875, 279876, 279877, 279878, 279879, 279880, 279881, 279882, 279883, 279884, 279885, 279886, 279887, 279888) c6( ) c7(bN, T, aRp, qe, bbM, nX, btY, ha, atS, qK, ml, bw, ci, nA, ER, bz, MV, bt, xb, st, bA); #c1(NSD1) c2(XP_005266019) c3(histone-lysine N-methyltransferase, H3 lysine-36 and H4 lysine-20 specific isoform X9) c4( ) c5(279889) c6( ) c7(bN, T, aRp, qe, bbM, nX, btY, ha, atS, qK, ml, bw, ci, nA, ER, bz, MV, bt, xb, st, bA); #c1(NSD1) c2(XP_011532913) c3(histone-lysine N-methyltransferase, H3 lysine-36 and H4 lysine-20 specific isoform X1) c4(329076) c5(329077, 329078, 329079, 329080, 329081, 329082) c6( ) c7(bN, T, aRp, qe, bbM, nX, btY, ha, atS, qK, ml, bw, ci, nA, ER, bz, MV, bt, xb, st, bA); #c1(NSD1) c2(XP_011532915) c3(histone-lysine N-methyltransferase, H3 lysine-36 and H4 lysine-20 specific isoform X4) c4(329083) c5(329084, 329085, 329086, 329087, 329088, 329089) c6( ) c7(bN, T, aRp, qe, bbM, nX, btY, ha, atS, qK, ml, bw, ci, nA, ER, bz, MV, bt, xb, st, bA); #c1(NSDHL) c2(XP_011529480) c3(sterol-4-alpha-carboxylate 3-dehydrogenase, decarboxylating isoform X1) c4(324926, 324927) c5(324928, 324929, 324930, 324931, 324932, 324933, 324934, 324935, 324936, 324937, 324938, 324939, 324940, 324941, 324942, 324943, 324944, 324945, 324946, 324947, 324948, 324949) c6( ) c7(bex, bjq, cgc, cmd, nA, dr); #c1(NSF) c2(NP_006169) c3(vesicle-fusing ATPase) c4(162487) c5(162488, 162489, 162490, 162491, 162492, 162493, 162494, 162495, 162496, 162497, 162498, 162499) c6( ) c7(il, Or, jh, cm, act, bdq, an, bdr, he, VV, iJ); #c1(NSF) c2(XP_011523165) c3(vesicle-fusing ATPase isoform X2) c4(317696) c5( ) c6( ) c7(il, Or, jh, cm, act, bdq, an, bdr, he, VV, iJ); #c1(NSFL1C) c2(NP_001193665) c3(NSFL1 cofactor p47 isoform d) c4(82430, 82431) c5(82432, 82433, 82434, 82435, 82436, 82437, 82438, 82439, 82440, 82441, 82442, 82443) c6( ) c7(fe, bM, T, ra, iV); #c1(NSFL1C) c2(NP_057227) c3(NSFL1 cofactor p47 isoform a) c4(189592) c5(189593, 189594, 189595, 189596, 189597, 189598) c6( ) c7(fe, bM, T, ra, iV); #c1(NSFL1C) c2(NP_061327) c3(NSFL1 cofactor p47 isoform b) c4(200036) c5(200037, 200038, 200039, 200040, 200041, 200042) c6( ) c7(fe, bM, T, ra, iV); #c1(NSFL1C) c2(XP_006723657) c3(NSFL1 cofactor p47 isoform X8) c4(295275) c5(295276, 295277, 295278, 295279, 295280, 295281) c6( ) c7(fe, bM, T, ra, iV); #c1(NSG1) c2(NP_001274692) c3(neuron-specific protein family member 1 isoform a) c4(106675, 106676) c5(106677, 106678, 106679, 106680, 106681, 106682, 106683, 106684, 106685, 106686, 106687, 106688, 106689, 106690, 106691, 106692, 106693, 106694, 106695, 106696, 106697, 106698, 106699, 106700, 106701, 106702, 106703, 106704, 106705, 106706, 106707, 106708, 106709, 106710, 106711, 106712) c6( ) c7(hd, hL); #c1(NSMAF) c2(NP_001138244) c3(protein FAN isoform 2) c4(56128) c5(56129, 56130, 56131, 56132, 56133, 56134) c6( ) c7(T, cd, Y); #c1(NSMAF) c2(NP_003571) c3(protein FAN isoform 1) c4(139240) c5(139241, 139242, 139243, 139244, 139245, 139246, 139247, 139248) c6( ) c7(T, cd, Y); #c1(NSMCE2) c2(NP_775956) c3(E3 SUMO-protein ligase NSE2) c4(250792, 250793, 250794, 250795) c5(250796, 250797, 250798, 250799, 250800, 250801, 250802, 250803, 250804, 250805, 250806, 250807, 250808, 250809, 250810, 250811, 250812, 250813, 250814, 250815, 250816, 250817, 250818, 250819, 250820, 250821, 250822, 250823) c6( ) c7(ac, kS, K, hH, avx, ad); #c1(NSMF) c2(NP_001124441) c3(NMDA receptor synaptonuclear signaling and neuronal migration factor isoform a) c4(48913, 48914, 48915) c5(48916, 48917, 48918, 48919, 48920, 48921) c6( ) c7(aMP, zQ, cme, ci, UF, zP, yn, UE); #c1(NSMF) c2(NP_001124442) c3(NMDA receptor synaptonuclear signaling and neuronal migration factor isoform c) c4(48922, 48923) c5(48924, 48925, 48926, 48927, 48928, 48929, 48930, 48931, 48932, 48933, 48934, 48935) c6( ) c7(aMP, zQ, cme, ci, UF, zP, yn, UE); #c1(NSMF) c2(NP_001124443) c3(NMDA receptor synaptonuclear signaling and neuronal migration factor isoform d) c4(48936) c5(48937, 48938, 48939, 48940, 48941, 48942, 48943, 48944, 48945, 48946, 48947, 48948) c6( ) c7(aMP, zQ, cme, ci, UF, zP, yn, UE); #c1(NSMF) c2(NP_001171535) c3(NMDA receptor synaptonuclear signaling and neuronal migration factor isoform e) c4(70540) c5(70541, 70542, 70543, 70544, 70545, 70546) c6( ) c7(aMP, zQ, cme, ci, UF, zP, yn, UE); #c1(NSMF) c2(NP_056352) c3(NMDA receptor synaptonuclear signaling and neuronal migration factor isoform b) c4(187213, 187214, 187215) c5(187216, 187217, 187218, 187219, 187220, 187221, 187222, 187223, 187224, 187225, 187226, 187227, 187228, 187229, 187230, 187231, 187232) c6( ) c7(aMP, zQ, cme, ci, UF, zP, yn, UE); #c1(NSMF) c2(XP_011516799) c3(NMDA receptor synaptonuclear signaling and neuronal migration factor isoform X4) c4( ) c5(310243, 310244, 310245, 310246, 310247, 310248, 310249, 310250, 310251, 310252, 310253) c6( ) c7(aMP, zQ, cme, ci, UF, zP, yn, UE); #c1(NSRP1) c2(NP_001248396) c3(nuclear speckle splicing regulatory protein 1 isoform 2) c4( ) c5(93964, 93965, 93966, 93967, 93968, 93969, 93970, 93971, 93972, 93973, 93974) c6( ) c7(hF, fr, es); #c1(NSRP1) c2(NP_115517) c3(nuclear speckle splicing regulatory protein 1 isoform 1) c4(223028) c5(223029, 223030, 223031, 223032, 223033, 223034) c6( ) c7(hF, fr, es); #c1(NSRP1) c2(XP_011523647) c3(nuclear speckle splicing regulatory protein 1 isoform X1) c4(318141, 318142) c5(318143) c6( ) c7(hF, fr, es); #c1(NSUN2) c2(NP_001180384) c3(tRNA (cytosine(34)-C(5)- methyltransferase isoform 2) c4(73900, 73901) c5(73902, 73903, 73904, 73905, 73906, 73907, 73908, 73909, 73910, 73911, 73912, 73913) c6( ) c7(cmf, aW, aT, yu, K, Ea, nA, bZs, QK); #c1(NSUN2) c2(NP_060225) c3 (tRNA (cytosine(34)-C(5)-methyltransferase isoform 1) c4(194854) c5(194855, 194856, 194857, 194858, 194859, 194860) c6( ) c7(cmf, aW, aT, yu, K, Ea, nA, bZs, QK); #c1(NSUN3) c2(NP_071355) c3(putative methyltransferase NSUN3) c4(211180) c5(211181, 211182, 211183, 211184, 211185, 211186) c6( ) c7(iu); #c1(NSUN5) c2(NP_001161819) c3 (probable 28S rRNA (cytosine-C(5)-methyltransferase isoform 3) c4(66091, 66092) c5(66093, 66094, 66095, 66096, 66097, 66098, 66099, 66100) c6( ) c7(T, alC, aE, AO, qt, aW, r, aT, ci, zy, hF, eA); #c1(NSUNS) c2(NP_001161820) c3(probable 28S rRNA (cytosine-C(5)-methyltransferase isoform 4) c4(66101) c5(66102, 66103, 66104, 66105, 66106) c6( ) c7(T, alC, aE, AO, qt, aW, r, aT, ci, zy, hF, eA); #c1(NSUN5) c2(NP_060514) c3(probable 28S rRNA (cytosine-C(5)-methyltransferase isoform 2) c4( ) c5(196316, 196317, 196318, 196319, 196320) c6( ) c7(T, alC, aE, AO, qt, aW, r, aT, ci, zy, hF, eA); #c1(NSUNS) c2(NP_683759) c3 (probable 28S rRNA (cytosine-C(5))-methyltransferase isoform 1) c4(242546) c5(242547, 242548, 242549, 242550, 242551, 242552) c6( ) c7(T, alC, aE, AO, qt, aW, r, aT, ci, zy, hF, eA); #c1(NSUN7) c2(NP_078953) c3(putative methyltransferase NSUN7) c4(216242) c5(216243, 216244, 216245, 216246, 216247, 216248) c6( ) c7(gU); #c1(NTSC) c2(NP_001239306) c3(5′(3′)-deoxyribonucleotidase, cytosolic type isoform 2) c4(88405) c5(88406, 88407, 88408, 88409, 88410, 88411) c6( ) c7(bt); #c1(NT5C) c2(NP_055410) c3(5′(3′)-deoxyribonucleotidase, cytosolic type isoform 1) c4(180908) c5(180909, 180910, 180911, 180912, 180913, 180914) c6( ) c7(bt); #c1(NTSC1B) c2(NP_001002006) c3(cytosolic 5′-nucleotidase lB isoform 1) c4(19334) c5(19335, 19336, 19337, 19338, 19339, 19340) c6( ) c7(e, ix, Cr, ac); #c1(NTSC1B) c2(NP_001186016) c3(cytosolic 5′-nucleotidase 1B isoform 4) c4(76869) c5(76870, 76871, 76872, 76873, 76874, 76875) c6( ) c7(e, iX, Cr, ac); #c1(NTSC1B) c2(NP_150278) c3(cytosolic 5′-nucleotidase 18 isoform 2) c4(228015) c5(228016, 228017, 228018, 228019, 228020, 228021) c6( ) c7(e, iX, Cr, ac); #c1(NTSC1B-RDH14) c2(NP_001186032) c3(NTSC1B-RDH14 protein isoform 1) c4(76902) c5(76903, 76904, 76905, 76906, 76907, 76908) c6( ) c7(iX); #c1(NTSC1B-RDH14) c2(NP_001186033) c3(NTSC1B-RDH14 protein isoform 2) c4(76909) c5(76910, 76911, 76912, 76913, 76914, 76915) c6( ) c7(iX); #c1(NTSC2) c2(NP_036361) c3(cytosolic purine 5′-nucleotidase) c4(173227) c5(173228, 173229, 173230, 173231, 173232, 173233, 173234, 173235, 173236, 173237) c6( ) c7(bL, Kd, T, nD, lD, oe, cz, bB, wV, v, aje, eW, x, av, aT, cmg, ly, d, aW, Y, oV, tv, tu, as, cM, nC, adA, lx, ck, Dy, eM, nw, k, lC, bt); #c1(NTSC2) c2(XP_005269696) c3(cytosolic purine 5′-nucleotidase isoform X7) c4(282720) c5(282721, 282722, 282723, 282724, 282725, 282726) c6( ) c7(bL, Kd, T, nD, lD, oe, cz, bB, wV, v, aje, eW, x, av, aT, cmg, ly, d, aW, Y, oV, tv, tu, as, cM, nC, adA, lx, ck, Dy, eM, nw, k, lC, bt); #c1(NTSC2) c2(XP_006717787) c3(cytosolic purine 5′-nucleotidase isoform X9) c4( ) c5(291362, 291363, 291364, 291365, 291366, 291367) c6( ) c7(bL, Kd, T, nD, lD, oe, cz, bB, wV, v, aje, eW, x, av, aT, cmg, ly, d, aW, Y, oV, tv, tu, as, cM, nC, adA, lx, ck, Dy, eM, nw, k, lC, bt); #c1(NTSC2) c2(XP_011537836) c3(cytosolic purine 5′-nucleotidase isoform X2) c4(335330, 335331) c5( ) c6( ) c7(bL, Kd, T, nD, lD, oe, cz, bB, wV, v, aje, eW, x, av, aT, cmg, ly, d, aW, Y, oV, tv, tu, as, cM, nC, adA, lx, ck, Dy, eM, nw, k, lC, bt); #c1(NT5C2) c2(XP_011537839) c3(cytosolic purine 5′-nucleotidase isoform X8) c4(335332, 335333) c5(335334, 335335, 335336, 335337, 335338, 335339, 335340, 335341, 335342, 335343, 335344, 335345, 335346, 335347, 335348, 335349, 335350, 335351) c6( ) c7(bL, Kd, T, nD, lD, oe, cz, bB, wV, v, aje, eW, x, av, aT, cmg, ly, d, aW, Y, oV, tv, tu, as, cM, nC, adA, lx, ck, Dy, eM, nw, k, lC, bt); #c1(NTSC3A) c2(NP_001002010) c3(cytosolic 5′-nucleotidase 3A isoform 1) c4(19341, 19342) c5(19343, 19344, 19345, 19346, 19347, 19348) c6( ) c7(bL, T, aj, mC, J, eW, AW, aE, dV, aT, lQ, cmh, pi, uQ, aW, eZ, S, cm, zz, aHG, dB, WP, byG, Nl, Xa, fL, Cl, ei, bt, WX, B); #c1(NTSC3A) c2(NP_001159590) c3(cytosolic 5′-nucleotidase 3A isoform 3) c4(64410) c5(64411, 64412, 64413, 64414, 64415, 64416, 64417, 64418, 64419, 64420, 64421, 64422, 64423, 64424, 64425) c6( ) c7(bL, T, aj, mC, J, eW, AW, aE, dV, aT, lQ, cmh, pi, uQ, aW, eZ, S, cm, zz, aHG, dB, WP, byG, Nl, Xa, fL, Cl, ei, bt, WX, B); #c1(NTSC3A) c2(NP_057573) c3(cytosolic 5′-nucleotidase 3A isoform 2) c4(191396) c5(191397, 191398, 191399, 191400, 191401, 191402, 191403, 191404, 191405, 191406, 191407, 191408) c6( ) c7(bL, T, aj, mC, J, eW, AW, aE, dV, aT, lQ, cmh, pi, uQ, aW, eZ, S, cm, zz, aHG, dB, WP, byG, Nl, Xa, FL, Cl, ei, bt, WX, B); #c1(NT5DC1) c2(NP_689942) c3(5′-nucleotidase domain-containing protein 1) c4(244426, 244427, 244428) c5(244429, 244430, 244431, 244432, 244433, 244434) c6( ) c7(Y); #c1(NT5DC1) c2(XP_006715441) c3(5′-nucleotidase domain-containing protein 1 isoform X2) c4( ) c5(289566, 289567, 289568, 289569, 289570, 289571, 289572, 289573, 289574, 289575, 289576, 289577, 289578, 289579, 289580, 289581) c6( ) c7(Y); #c1(NT5DC3) c2(NP_001026871) c3(5′-nucleotidase domain-containing protein 3) c4(29736) c5(29737, 29738, 29739, 29740, 29741, 29742) c6( ) c7(bt, aC, jh); #c1(NTSE) c2(NP_001191742) c3(5′-nucleotidase isoform 2 preproprotein) c4(81635, 81636, 81637) c5(81638, 81639, 81640, 81641, 81642, 81643, 81644, 81645, 81646, 81647, 81648, 81649, 81650, 81651, 81652, 81653, 81654, 81655) c6( ) c7(T, dA, aD, e, aA, cz, n, bB, fo, zx, BF, V, ai, eA, Co, aT, io, h, ji, bq, aOh, zy, hB, Cr, cB, jf, aW, Y, jP, fQ, hp, zC, S, iV, nr, t, bw, zz, LB, ck, bE, lV, mG, cj, at, nL, rH, lS, y, bt, zA, fl); #c1(NT5E) c2(NP_002517) c3(5′-nucleotidase isoform 1 preproprotein) c4(129457, 129458, 129459) c5(129460, 129461, 129462, 129463, 129464, 129465, 129466, 129467, 129468, 129469, 129470, 129471, 129472, 129473, 129474, 129475, 129476, 129477) c6( ) c7(T, dA, aD, e, aA, cz, n, bB, fo, zx, BF, V, ai, eA, Co, aT, io, h, ji, bq, aOh, zy, hB, Cr, cB, jf, aW, Y, jP, fQ, hp, zC, S, iV, nr, t, bw, zz, LB, ck, bE, lV, mG, cj, at, nL, rH, lS, y, bt, zA, fl); #c1(NT5M) c2(NP_064586) c3(5′(3′)-deoxyribonucleotidase, mitochondrial precursor) c4(203119) c5(203120, 203121, 203122, 203123, 203124, 203125, 203126) c6( ) c7(dr, fd); #c1(NT5M) c2(XP_005256788) c3(5′(3′)-deoxyribonucleotidase, mitochondrial isoform X1) c4(274511) c5( ) c6( ) c7(dr, fd); #c1(NT5M) c2(XP_005256790) c3(5′(3′)-deoxyribonucleotidase, mitochondrial isoform X4) c4( ) c5(274512, 274513, 274514, 274515, 274516) c6( ) c7(dr, fd); #c1(NT5M) c2(XP_011522267) c3(5′(3′)-deoxyribonucleotidase, mitochondrial isoform X7) c4( ) c5(316945, 316946) c6( ) c7(dr, fd); #c1(NTAN1) c2(NP_775745) c3(protein N-terminal asparagine amidohydrolase isoform 1) c4(250147, 250148) c5(250149, 250150, 250151, 250152, 250153, 250154, 250155, 250156, 250157, 250158, 250159, 250160) c6( ) c7(k, cz); #c1(NTF3) c2(NP_001096124) c3(neurotrophin-3 isoform 1 preproprotein) c4(41077, 41078) c5(41079, 41080, 41081, 41082, 41083, 41084, 41085, 41086, 41087) c6( ) c7(bM, cZ, T, ul, hl, Ol, fG, bO, ti, aj, bS, Vd, ct, fu, PJ, cmi, aR, J, ad, dP, eA, Mg, aE, uT, sO, fm, bW, Fo, HC, M, bK, K, bU, dd, he, rE, ahE, dF, af, cB, jf, aW, Y, gl, ahl, as, iV, dw, Z, kk, f, cm, yX, bN, hq, ek, bF, ac, c, cA, dB, asl, eM, aHB, U, at, fD, bt, aqD, qc); #c1(NTF3) c2(NP_002518) c3(neurotrophin-3 isoform 2 preproprotein) c4( ) c5(129478, 129479, 129480, 129481, 129482, 129483, 129484) c6( ) c7(bM, cZ, T, ul, hl, Ol, fG, bO, ti, aj, bS, Vd, ct, fu, PJ, cmi, aR, J, ad, dP, eA, Mg, aE, uT, sO, fm, bW, Fo, HC, M, bK, K, bU, dd, he, rE, ahE, dF, af, cB, jf, aW, Y, gl, ahl, as, iV, dw, Z, kk, f, cm, yX, bN, hq, ek, bF, ac, c, cA, dB, asl, eM, aHB, U, at, fD, bt, aqD, qc); #c1(NTF4) c2(XP_011525310) c3(neurotrophin-4 isoform X2) c4(319770) c5( ) c6( ) c7(dP, bM, hl, c, pZ, dH, fm, Y, aW, sa, HC, aT, cm, bK, nA, eE, px, lJ, cmj, ad, bN); #c1(NTF4) c2(XP_011525312) c3(neurotrophin-4 isoform X1) c4( ) c5(319771, 319772, 319773, 319774, 319775, 319776, 319777) c6( ) c7(dP, bM, hl, c, pZ, dH, fm, Y, aW, sa, HC, aT, cm, bK, nA, eE, px, lJ, cmj, ad, bN); #c1(NTHL1) c2(NP_002519) c3(endonuclease III-like protein 1) c4(129485) c5(129486, 129487, 129488, 129489, 129490, 129491) c6( ) c7(bF, qw, T, rJ, r, aA, aF, V, q, Qe); #c1(NTM) c2(NP_001041674) c3(neurotrimin isoform 2 precursor) c4(34272) c5(34273, 34274, 34275, 34276, 34277, 34278) c6( ) c7(ac, dH, ig, bO, fK, Wb, ahg, be, he, cd); #c1(NTM) c2(NP_001137530) c3(neurotrimin isoform 3 precursor) c4(55986) c5(55987, 55988, 55989, 55990, 55991, 55992) c6( ) c7(ac, dH, ig, bO, fK, Wb, ahg, be, he, cd); #c1(NTM) c2(NP_001137531) c3(neurotrimin isoform 4 precursor) c4(55993) c5(55994, 55995, 55996, 55997, 55998, 55999) c6( ) c7(ac, dH, ig, bO, fK, Wb, ahg, be, he, cd); #c1(NTM) c2(NP_057606) c3(neurotrimin isoform 1 precursor) c4(19/714) c5(191715, 191716, 191717, 191718, 191719, 191720) c6( ) c7(ac, dH, ig, bO, fK, Wb, ahg, be, he, cd); #c1(NTM) c2(XP_005271639) c3(neurotrimin isoform X4) c4(283508) c5(283509, 283510, 283511, 283512, 283513, 283514) c6( ) c7(ac, dH, ig, bO, fK, Wb, ahg, be, he, cd); #c1(NTM) c2(XP_006718918) c3(neurotrimin isoform X6) c4(292081) c5(292082, 292083, 292084, 292085, 292086, 292087) c6( ) c7(ac, dH, ig, bO, fK, Wb, ahg, be, he, cd); #c1(NTN1) c2(XP_006721658) c3(netrin-1 isoform X1) c4(294157, 294158) c5(294159, 294160, 294161, 294162, 294163, 294164, 294165, 294166, 294167, 294168, 294169, 294170, 294171, 294172, 294173, 294174, 294175, 294176, 294177, 294178, 294179, 294180, 294181, 294182, 294183, 294184, 294185, 294186) c6(294187, 294188, 294189, 294190, 294191, 294192, 294193, 294194) c7(T, dD, ea, e, bB, j, eq, C, fr, q, av, aT, cy, K, ae, Qe, jf, d, B, aW, Y, r, zC, cl, t, cm, bz, eM, asl, bE, iA, hL, m, y, X); #c1(NTN4) c2(NP_067052) c3(netrin-4 precursor) c4(208752, 208753, 208754) c5(208755, 208756, 208757, 208758, 208759, 208760, 208761, 208762, 208763, 208764, 208765, 208766, 208767, 208768, 208769, 208770, 208771, 208772, 208773) c6( ) c7(T, d, hb, e, aT, ad, cY); #c1(NTNG1) c2(XP_011539324) c3(netrin-G1 isoform X4) c4(337406) c5(337407, 337408, 337409, 337410, 337411, 337412) c6( ) c7(Qi, iX, r, bK, fL, nA, an, fq, q, bN); #c1(NTNG1) c2(XP_011539326) c3(netrin-G1 isoform X6) c4(337413, 337414) c5(337415, 337416, 337417, 337418, 337419, 337420, 337421, 337422, 337423, 337424, 337425, 337426) c6( ) c7(Qi, iX, r, bK, fL, nA, an, fq, q, bN); #c1(NTNG1) c2(XP_011539327) c3(netrin-G1 isoform X8) c4( ) c5(337427, 337428, 337429, 337430, 337431, 337432, 337433, 337434, 337435, 337436) c6( ) c7(Qi, iX, r, bK, fL, nA, an, fq, q, bN); #c1(NTNG2) c2(NP_115925) c3(netrin-G2 precursor) c4(224711, 224712, 224713) c5(224714, 224715, 224716, 224717, 224718, 224719, 224720, 224721, 224722, 224723, 224724, 224725, 224726, 224727, 224728, 224729, 224730, 224731) c6( ) c7(Qi, eZ, dL, bK, nA, dL, bN); #c1(NTNG2) c2(XP_011517412) c3(netrin-G2 isoform X7) c4(311123) c5(311124, 311125, 311126, 311127, 311128, 311129) c6( ) c7(Qi, eL, dL, bK, nA, eL, bN); #c1(NTPCR) c2(NP_115700) c3(cancer-related nucleoside-triphosphatase) c4(223632, 223633) c5(223634, 223635, 223636, 223637, 223638, 223639, 223640, 223641, 223642, 223643, 223644, 223645) c6( ) c7(vi); #c1(NTRK1) c2(NP_001007793) c3(high affinity nerve growth factor receptor isoform 3) c4(22855) c5(22856, 22857, 22858, 22859, 22860, 22861) c6( ) c7(lD, bM, T, aC, Tb, fR, if, ci, YD, Vp, bv, aE, pX, Ou, K, hH, eL, oP, aW, g, as, eE, eF, cm, Qt, zV, bDr, nB, eM, iA, iT, cj, QF, m, aHk, ik, cZ, gU, lT, wu, yG, n, aj, fr, ct, nA, ml, KL, ft, TK, gd, cU, dL, dd, dx, cml, fe, kp, yq, dw, yR, cjP, bP, dB, hk, bjx, uz, B, bL, Dw, Lx, hz, bX, bDq, V, q, J, eA, asG, uT, lg, nv, Qg, qK, aT, dz, bK, bq, ahE, ae, af, aeY, jf, ak, hM, iF, s, vT, iU, Y, alJ, r, ahl, en, t, ik, cmk, vk, bN, tR, ck, Ta, nM, hc, cjR, JL, PN, bt, VB, ao, Pt, aFa, iW, Me, jh, bS, oH, ad, BY, dP, Tg, hd, Db, ji, wm, yh, ahk, ako, nJ, fb, TC, d, lR, et, vj, ff, cr, eZ, aeV, aF, Z, bz, Hv, dr, Hw, eU, pc, xZ, bE, Tc); #c1(NTRK1) c2(NP_001012331) c3(high affinity nerve growth factor receptor isoform 1 precursor) c4(24882) c5(24883, 24884, 24885, 24886, 24887, 24888) c6( ) c7(lD, bM, T, aC, Tb, fR, if, ci, YD, Vp, bv, aE, pX, Ou, K, hH, eL, oP, aW, g, as, eE, eF, cm, Qt, zV, bDr, nB, eM, iA, iT, cj, QF, m, aHk, ik, cZ, gU, lT, wu, yG, n, aj, fr, ct, nA, ml, KL, ft, TK, gd, cU, dL, dd, dx, cml, fe, kp, yq, dw, yR, cjP, bP, dB, hk, bjx, uz, B, bL, Dw, Lx, hz, bX, bDq, V, q, J, eA, asG, uT, lg, nv, Qg, qK, aT, dz, bK, bq, ahE, ae, af, aeY, jf, ak, hM, iF, s, vT, iU, Y, alJ, r, ahl, en, t, iK, cmk, vk, bN, tR, ck, Ta, nM, hc, cjR, JL, PN, bt, VB, ao, Pt, aFa, iW, Me, jh, bS, oH, ad, BY, dP, Tg, hd, Db, ji, wm, yh, ahK, ako, nJ, fb, TC, d, lR, et, vj, ff, cr, eZ, aeV, aF, Z, bz, Hv, dr, Hw, eU, pc, xZ, bE, Tc); #c1(NTRK1) c2(NP_002520) c3(high affinity nerve growth factor receptor isoform 2 precursor) c4(129492) c5(129493, 129494, 129495, 129496, (29497, 129498) c6( ) c7(lD, bM, T, aC, Tb, fR, if, ci, YD, Vp, bv, aE, pX, Ou, K, hH, eL, oP, aW, g, as, eE, eF, cm, Qt, zV, bDr, nB, eM, iA, iT, cj, QF, m, aHk, ik, cZ, gU, lT, wu, yG, n, aj, fr, ct, nA, ml, KL, ft, TK, gd, cU, dL, dd, dx, cml, fe, kp, yq, dw, yR, cjP, bP, dB, hk, bjx, uz, B, bL, Dw, Lx, hz, bX, bDq, V, q, J, eA, asG, uT, lg, nv, Qg, qK, aT, dz, bk, bq, ahE, ae, af, aeY, jf, ak, hM, iF, s, vT, iU, Y, aLJ, r, ahl, en, t, ik, cmk, vk, bN, tR, ck, Ta, nM, hc, cjR, JL, PN, bt, VB, ao, Pt, aFa, iW, Me, jh, bS, oH, ad, BY, dP, Tg, hd, Db, ji, wm, yh, ahK ako, nJ, fb, TC, d, lR, et, vj, ff, cr, eZ, aeV, aF, Z, bz, Hv, dr, Hw, eU, pc, xZ, bE, Tc); #c1(NTRK2) c2(NP_001018075) c3(BDNF/NT-3 growth factors receptor isoform d precursor) c4(27093) c5(27094, 27095, 27096, 27097, 27098, 27099) c6( ) c7(Fv, T, uk, id, Tb, lr, bO, bB, bS, v, ct, il, qi, q, eA, dP, iu, Tg, ft, aE, yt, aqJ, iX, pX, qK, aql, cy, bK, K, ji, oi, he, ahK, cmm, af, rE, ak, A, Gv, Y, r, cmn, cu, g, cl, dw, ajz, t, bz, Z, as, bN, hq, amt, cm, Hw, aqr, Hp, auo, cA, bE, aqM, ZT, nG, Ei, m, cB, B, qc); #c1(NTRK2) c2(NP_001018076) c3(BDNF/NT-3 growth factors receptor isoform e precursor) c4(27100) c5(27101, 27102, 27103, 27104, 27105, 27106) c6( ) c7(Fv, T, uk, id, Tb, lr, bO, bB, bS, v, ct, il, qi, q, eA, dP, iu, Tg, ft, aE, yt, aqJ, iX, pX, qK, aql, cy, bK, K, ji, oi, he, ahK, cmm, af, rE, ak, A, Gv, Y, r, cmn, cu, g, cl, dw, ajz, t, bz, Z, as, bN, hq, amt, cm, Hw, aqr, Hp, auo, cA, bE, aqM, ZT, nG, Ei, m, cB, B, qc); #c1(NTRK2) c2(XP_005252061) c3(BDNF/NT-3 growth factors receptor isoform X3) c4(271511) c5(271512, 271513, 271514, 271515, 271516, 271517, 271518, 271519, 271520, 271521) c6( ) c7(Fv, T, uk, id, Tb, lr, bO, bB, bS, v, ct, il, qi, q, eA, dP, iu, Tg, ft, aE, yt, aqJ, iX, pX, qK, aql, cy, bK, K, ji, oi, he, ahk, cmm, af, rE, ak, A, Gv, Y, r, cmn, cu, g, cl, dw, ajz, t, bz, Z, as, bN, hq, amt, cm, Hw, aqr, Hp, auo, cA, bE, aqM, ZT, nG, Ei, m, cB, B, qc); #c1(NTRK2) c2(XP_005252064) c3(BDNF/NT-3 growth factors receptor isoform X4) c4(271522, 271523) c5(271524, 271525, 271526, 271527, 271528, 271529, 271530, 271531, 271532, 271533, 271534, 271535, 271536, 271537, 271538, 271539) c6( ) c7(Fv, T, uk, id, Tb, lr, bO, bB, bS, v, ct, il, qi, q, eA, dP, iu, Tg, ft, aE, yt, aqJ, iX, pX, qK, aql, cy, bK, K, ji, oi, he, ahK, cmm, af, rE, ak, A, Gv, Y, r, cmn, cu, g, cl, dw, ajz, t, bz, Z, as, bN, hq, amt, cm, Hw, aqr, Hp, auo, cA, bE, aqM, ZT, nG, Ei, m, cB, B, qc); #c1(NTRK2) c2(XP_011517021) c3(BDNF/NT-3 growth factors receptor isoform X5) c4(310511) c5(310512, 310513, 310514, 310515, 310516, 310517, 310518, 310519, 310520, 310521) c6( ) c7(Fv, T, uk, id, Tb, lr, bO, bB, bS, v, ct, il, qi, q, eA, dP, iu, Tg, ft, aE, yt, aqJ, iX, pX, qK, aql, cy, bK, K, ji, oi, he, ahK, cmm, af, rE, ak, A, Gv, Y, r, cmn, cu, g, cl, dw, ajz, t, bz, Z, as, bN, hq, amt, cm, Hw, aqr, Hp, auo, cA, bE, aqM, ZT, nG, Ei, m, cB, B, qc); #c1(NTRK3) c2(NP_001007157) c3(NT-3 growth factor receptor isoform c precursor) c4(22514) c5(22515, 22516, 22517, 22518, 22519, 22520, 22521, 22522, 22523, 22524) c6( ) c7(T, aC, aHx, bO, cz, ww, ci, AO, avD, q, ad, eA, afb, dP, hd, iX, qK, aql, aT, in, bq, rE, ae, ahE, dx, Yl, jf, tb, s, aW, Y, boG, r, ahl, g, Z, cm, bN, hq, vU, es, cA, nB, aHB, nG, Kg, bt, cB, qc); #c1(NTRK3) c2(NP_001012338) c3(NT-3 growth factor receptor isoform a precursor) c4(24889, 24890) c5(24891, 24892, 24893, 24894, 24895, 24896, 24897, 24898, 24899, 24900, 24901, 24902, 24903) c6( ) c7(T, aC, aHx, bO, cz, ww, ci, AO, avD, q, ad, eA, afb, dP, hd, iX, qK, aql, aT, in, bq, rE, ae, ahE, dx, Yl, jf, tb, s, aW, Y, boG, r, ahl, g, Z, cm, bN, hq, vU, es, cA, nB, aHB, nG, Kg, bt, cB, qc); #c1(NTRK3) c2(NP_002521) c3(NT-3 growth factor receptor isoform b precursor) c4(129499) c5(129500, 129501, 129502, 129503, 129504, 129505, 129506, 129507, 129508, 129509) c6( ) c7(T, aC, aHx, bO, cz, ww, ci, AO, avD, q, ad, eA, afb, dP, hd, iX, qK, aql, aT, in, bq, rE, ae, ahE, dx, Yl, jf, tb, s, aW, Y, boG, r, ahl, g, Z, cm, bN, hq, vU, es, cA, nB, aHB, nG, Kg, bt, cB, qc); #c1(NTRK3) c2(XP_006720608) c3(NT-3 growth factor receptor isoform X5) c4(293587) c5(293588) c6( ) c7(T, aC, aHx, bO, cz, ww, ci, AO, avD, q, ad, eA, afb, dP, hd, iX, qK, aql, aT, in, bq, rE, ae, ahE, dx, Yl, jf, tb, s, aW, Y, boG, r, ahl, g, Z, cm, bN, hq, vU, es, cA, nB, aHB, nG, Kg, bt, cB, qc); #c1(NTRK3) c2(XP_011519938) c3(NT-3 growth factor receptor isoform X6) c4(314728) c5(314729, 314730, 314731, 314732, 314733, 314734, 314735, 314736, 314737, 314738) c6( ) c7(T, aC, aHx, bO, cz, ww, ci, AO, avD, q, ad, eA, afb, dP, hd, iX, qK, aql, aT, in, bq, rE, ae, ahE, dx, Yl, jf, tb, s, aW, Y, boG, r, ahl, g, Z, cm, bN, hq, vU, es, cA, nB, aHB, nG, Kg, bt, cB, qc); #c1(NTS) c2(NP_006174) c3(neurotensin/neuromedin N preproprotein) c4(162500, 162501, 162502) c5(162503, 162504, 162505, 162506, 162507, 162508, 162509, 162510, 162511, 162512, 162513, 162514, 162515, 162516, 162517, 162518, 162519, 162520, 162521, 162522, 162523, 162524, 162525) c6(162526) c7(T, tp, yY, cmo, cz, ti, bB, Fb, ci, C, q, ad, eJ, ft, ib, aE, lv, aT, bK, K, ji, an, fb, ae, af, Yi, tN, yo, s, nO, ev, yL, ff, cr, r, fe, gl, tv, aq, tu, gu, en, g, eE, t, bz, Dm, op, bN, YR, Ta, ok, kP, eM, bE, hc, vA, zl, xg, B, hS); #c1(NTSR1) c2(NP_002522) c3(neurotensin receptor type 1) c4(129510) c5(129511, 129512, 129513, 129514, 129515, 129516) c6( ) c7(T, bO, cz, bB, bS, uu, ci, q, ad, eW, av, ib, aE, sO, he, fb, tN, VX, s, vj, ff, cr, r, fe, en, g, eE, bz, Ta, cA, eM, bE, hE); #c1(NTSR2) c2(NP_036476) c3(neurotensin receptor type 2) c4(174140) c5(174141, 174142, 174143, 174144, 174145, 174146) c6( ) c7(yo); #c1(NUAK1) c2(NP_055655) c3(NUAK family SNFI-like kinase 1) c4(182553, 182554) c5(182555, 182556, 182557, 182558, 182559, 182560, 182561, 182562, 182563, 182564, 182565, 182566) c6( ) c7(T, pe, aW, Y, jh, r, aT, fd, q, B, cp); #c1(NUAK2) c2(NP_112214) c3(NUAK family SNFI-like kinase 2) c4(220855) c5(220856, 220857, 220858, 220859, 220860, 220861) c6( ) c7(T, Y, m, t, kG, vi); #c1(NUB1) c2(NP_001230280) c3(NEDD8 ultimate buster 1 isoform 1) c4(86184) c5(86185, 86186, 86187, 86188, 86189, 86190, 86191) c6( ) c7(T, fr); #c1(NUB1) c2(NP_057202) c3(NEDD8 ultimate buster 1 isoform 2) c4(189456) c5(189457, 189458, 189459, 189460, 189461, 189462, 189463, 189464, 189465, 189466, 189467, 189468, 189469) c6( ) c7(T, fr); #c1(NUB1) c2(XP_005250068) c3(NEDD8 ultimate buster 1 isoform X4) c4(270061, 270062, 270063, 270064) c5(270065, 270066, 270067, 270068, 270069, 270070, 270071, 270072, 270073, 270074, 270075, 270076, 270077, 270078, 270079, 270080, 270081, 270082, 270083, 270084) c6( ) c7(T, fr); #c1(NUBP1) c2(NP_001265435) c3(cytosolic Fe-S cluster assembly factor NUBP1 isoform 2) c4(98594) c5(98595, 98596, 98597, 98598, 98599, 98600) c6( ) c7(at, cm, xg, K, zU); #c1(NUBP1) c2(NP_002475) c3(cytosolic Fe-S cluster assembly factor NUBP1 isoform 1) c4(129151) c5(129152, 129153, 129154, 129155, 129156, 129157) c6( ) c7(at, cm, xg, K, zU); #c1(NUBP2) c2(NP_036357) c3(cytosolic Fe-S cluster assembly factor NUBP2 isoform 1) c4(173204, 173205) c5(173206, 173207, 173208, 173209, 173210, 173211) c6( ) c7(at, btE, dL, btk, zU); #c1(NUBP2) c2(XP_005255087) c3(cytosolic Fe-S cluster assembly factor NUBP2 isoform X3) c4( ) c5(273531, 273532, 273533, 273534, 273535) c6( ) c7(at, btE, dL, btk, zU); #c1(NUBP2) c2(XP_011520640) c3(cytosolic Fe-S cluster assembly factor NUBP2 isoform X2) c4( ) c5(315542) c6( ) c7(at, btE, dL, btk, zU); #c1(NUBPL) c2(NP_001188502) c3(iron-sulfur protein NUBPL isoform 2) c4( ) c5(79225, 79226, 79227, 79228, 79229, 79230) c6( ) c7(aM, cz, bxk, he, kC, V); #c1(NUBPL) c2(NP_079428) c3(iron-sulfur protein NUBPL isoform 1 precursor) c4(218078, 218079) c5(218080, 218081, 218082, 218083, 218084, 218085) c6( ) c7(aM, cz, bxk, he, kC, V); #c1(NUCB1) c2(NP_006175) c3(nucleobindin-1 precursor) c4(162527, 162528, 162529) c5(162530, 162531, 162532, 162533, 162534, 162535, 162536, 162537, 162538, 162539, 162540, 162541, 162542, 162543, 162544, 162545, 162546, 162547, 162548, 162549, 162550, 162551, 162552, 162553, 162554, 162555, 162556) c6( ) c7(vi); #c1(NUCB2) c2(NP_005004) c3(nucleobindin-2 precursor) c4( ) c5(151995, 151996, 151997, 151998, 151999, 152000) c6( ) c7(bL, T, e, aw, aA, n, aj, bS, afE, ci, dG, v, nS, ad, aT, h, jy, K, qM, he, ae, aW, af, ac, ei); #c1(NUCB2) c2(XP_011518423) c3(nucleobindin-2 isoform X1) c4(312437, 312438) c5( ) c6( ) c7(bL, T, e, aw, aA, n, aj, bS, afE, ci, dG, v, nS, ad, aT, h, jy, K, qM, he, ae, aW, aF, ac, ei); #c1(NUCB2) c2(XP_011518432) c3(nucleobindin-2 isoform X2) c4( ) c5(312439, 312440, 312441, 312442, 312443, 312444) c6( ) c7(bL, T, e, aw, aA, n, aj, bS, afE, ci, dG, v, nS, ad, aT, h, jy, K, qM, he, ae, aW, af, ac, ei); #c1(NUCB2) c2(XP_011518433) c3(nucleobindin-2 isoform X3) c4(312445, 312446) c5( ) c6( ) c7(bL, T, e, aw, aA, n, aj, bS, afE, ci, dG, v, nS, ad, aT, h, jy, K, qM, he, ae, aW, af, ac, ei); #c1(NUCB2) c2(XP_011518434) c3(nucleobindin-2 isoform X4) c4(312447) c5( ) c6( ) c7(bL, T, e, aw, aA, n, aj, bS, afE, ci, dG, v, nS, ad, aT, h, jy, K, qM, he, ae, aW, aF, ac, ei); #c1(NUCB2) c2(XP_011518435) c3(nucleobindin-2 isoform X5) c4(312448, 312449, 312450, 312451) c5(312452, 312453, 312454, 312455, 312456, 312457, 312458, 312459, 312460, 312461, 312462, 312463, 312464, 312465, 312466, 312467, 312468, 312469, 312470, 312471, 312472, 312473, 312474, 312475, 312476, 312477, 312478, 312479, 312480, 312481, 312482, 312483, 312484, 312485, 312486, 312487, 312488, 312489, 312490, 312491, 312492, 312493, 312494, 312495, 312496, 312497, 312498, 312499, 312500, 312501) c6( ) c7(bL, T, e, aw, aA, n, aj, bS, afE, ci, dG, v, nS, ad, aT, h, jy, K, qM, he, ae, aW, aF, ac, ei); #c1(NUCKS1) c2(NP_073568) c3(nuclear ubiquitous casein and cyclin-dependent kinase substrate 1) c4(212875, 212876) c5(212877, 212878, 212879, 212880, 212881, 212882) c6( ) c7(n, T, q, an, r); #c1(NUCKS1) c2(XP_005245510) c3(nuclear ubiquitous casein and cyclin-dependent kinase substrate 1 isoform X1) c4( ) c5(266670, 266671, 266672, 266673, 266674, 266675) c6( ) c7(n, T, q, an, r); #c1(NUDC) c2(XP_011538831) c3(nuclear migration protein nudC isoform X1) c4(336806, 336807) c5(336808, 336809, 336810, 336811, 336812, 336813) c6( ) c7(ck, bt, k, ad, x); #c1(NUDC) c2(XP_011538832) c3(nuclear migration protein nudC isoform X2) c4( ) c5(336814, 336815, 336816, 336817, 336818) c6( ) c7(ck, bt, k, ad, x); #c1(NUDCD1) c2(NP_001121683) c3(nudC domain-containing protein 1 isoform 2) c4(46048, 46049) c5(46050, 46051, 46052, 46053, 46054, 46055, 46056) c6( ) c7(ck, ae, T, ac, iU, m, qN, t, bt, bz, ad); #c1(NUDCD1) c2(NP_116258) c3(nudC domain-containing protein 1 isoform 1) c4(226013) c5(226014, 226015, 226016, 226017, 226018, 226019) c6( ) c7(ck, ae, T, ac, iU, m, qN, t, bt, bz, ad); #c1(NUDCD3) c2(NP_056147) c3(nudC domain-containing protein 3) c4(185962) c5(185963, 185964, 185965, 185966, 185967, 185968) c6( ) c7(aq); #c1(NUDT1) c2(NP_945191) c3(7,8-dihydro-8-oxoguanine triphosphatase isoform p18) c4(260729) c5(260730, 260731, 260732, 260733, 260734, 260735, 260736, 260737, 260738, 260739, 260740) c6( ) c7(bF, T, ft, jz, A, aiB, eM, Gs, an, U, aT, r, ci, K, anW, q, rk, ad); #c1(NUDT1) c2(NP_945192) c3(7,8-dihydro-8-oxoguanine triphosphatase isoform p22) c4(260741, 260742) c5(260743, 260744, 260745, 260746, 260747, 260748, 260749, 260750, 260751, 260752, 260753, 260754, 260755, 260756, 260757, 260758, 260759) c6( ) c7(bF, T, ft, jz, A, aiB, eM, Gs, an, U, aT, r, ci, K, anW, q, rk, ad); #c1(NUDT10) c2(NP_001291892) c3(diphosphoinositol polyphosphate phosphohydrolase 3-alpha) c4(116491) c5(116492, 116493, 116494, 116495, 116496, 116497, 116498, 116499, 116500, 116501, 116502, 116503, 116504) c6( ) c7(lF, OV, xy, A, ON, bxf, qj, OM, OU, ad, yF, pC); #c1(NUDT11) c2(NP_060629) c3 (diphosphoinositol polyphosphate phosphohydrolase 3-beta) c4(196905) c5(196906, 196907, 196908, 196909, 196910, 196911) c6( ) c7(OV, Pa, vJ, il, OX, OS, CZ, FS, ON, ml, OM, pd, dr, ad, L); #c1(NUDT15) c2(NP_060753) c3(probable 8-oxo-dGTP diphosphatase NUDT15 isoform 1) c4(197602) c5(197603, 197604, 197605, 197606, 197607, 197608) c6( ) c7(hG); #c1(NUDT19) c2(NP_001099040) c3(nucleoside diphosphate-linked moiety X motif 19, mitochondrial precursor) c4(41771) c5(41772, 41773, 41774, 41775, 41776, 4/777) c6( ) c7(afR, xX, Be, nw, eQ, HU, ayj, nC); #c1(NUDT2) c2(NP_671702) c3(bis(5′-nucleosyl)-tetraphosphatase [asymmetrical]) c4(242058) c5(242059, 242060, 242061, 242062, 242063, 242064, 242065, 242066, 242067, 242068, 242069, 242070, 242071, 242072, 242073, 242074, 242075, 242076, 242077, 242078, 242079, 242080) c6( ) c7(qt, aW, aT, mo); #c1(NUDT21) c2(NP_008937) c3(cleavage and polyadenylation specificity factor subunit 5) c4(169662, 169663) c5(169664, 169665, 169666, 169667, 169668, 169669, 169670, 169671, 169672, 169673, 169674, 169675) c6( ) c7(d); #c1(NUDT3) c2(NP_006694) c3(diphosphoinositol polyphosphate phosphohydrolase 1) c4(167196) c5(167197, 167198, 167199, 167200, 167201, 167202) c6( ) c7(Jd, fq, v); #c1(NUDT6) c2(NP_009014) c3(nucleoside diphosphate-linked moiety X motif 6 isoform a) c4(170242, 170243) c5(170244, 170245, 170246, 170247, 170248, 170249, 170250, 170251) c6( ) c7(dP, jf, ft, pf, Y, ht, r, dL, j, qj, g, fy, eL, q, wa); #c1(NUDT6) c2(NP_932158) c3(nucleoside diphosphate-linked moiety X motif 6 isoform b) c4( ) c5(258826, 258827, 258828, 258829, 258830, 258831, 258832, 258833) c6( ) c7(dP, jf, ft, pf, Y, ht, r, dL, j, qj, g, fy, eL, q, wa); #c1(NUDT7) c2(NP_001099133) c3(peroxisomal coenzyme A diphosphatase NUDT7 isoform 1) c4(41796) c5(41797, 41798, 41799, 41800, 41801, 41802, 41803) c6( ) c7(aq); #c1(NUDT7) c2(NP_001230586) c3(peroxisomal coenzyme A diphosphatase NUDT7 isoform 3) c4(86365) c5(86366, 86367, 86368, 86369, 86370) c6( ) c7(aq); #c1(NUDT7) c2(NP_001230590) c3(peroxisomal coenzyme A diphosphatase NUDT7 isoform 2) c4(86394) c5(86395, 86396, 86397, 86398, 86399, 86400) c6( ) c7(aq); #c1(NUF2) c2(NP_663735) c3(kinetochore protein Nuf2) c4(241477, 241478, 241479, 241480, 241481, 241482) c5(241483, 241484, 241485, 241486, 241487, 241488, 241489, 241490, 241491, 241492, 241493, 241494, 241495, 241496, 241497, 241498, 241499, 241500, 241501, 241502, 241503, 241504, 241505, 241506, 241507, 241508, 241509, 241510, 241511, 241512, 241513, 241514, 241515, 241516, 241517, 241518, 241519, 241520, 241521, 241522) c6( ) c7(T, ac, eM, aw, r, aT, n, aA, cz, aF, fr, be, bu, g, bz); #c1(NUFIP1) c2(NP_036477) c3(nuclear fragile X mental retardation-interacting protein 1) c4(174147) c5(174148, 174149, 174150, 174151, 174152, 174153) c6( ) c7(QK, v); #c1(NUFIP2) c2(NP_065823) c3(nuclear fragile X mental retardation-interacting protein 2) c4(205692) c5(205693, 205694, 205695, 205696, 205697, 205698) c6( ) c7(bM, rz, z, bxn, Fo, ha, h, K, nA, xg, f, he, Ku, Kl, bvX, bN); #c1(NUGGC) c2(NP_001010906) c3(nuclear GTPase SLIP-GC) c4(24050, 24051) c5(24052, 24053, 24054, 24055, 24056, 24057, 24058, 24059, 24060, 24061, 24062, 24063) c6( ) c7(bAO); #c1(NUMA1) c2(NP_001273490) c3(nuclear mitotic apparatus protein 1 isoform 2) c4(105004, 105005, 105006, 105007, 105008, 105009) c5(105010, 105011, 105012, 105013, 105014, 105015, 105016, 105017, 105018, 105019, 105020, 105021, 105022, 105023, 105024, 105025, 105026, 105027, 105028, 105029, 105030, 105031, 105032, 105033, 105034, 105035, 105036, 105037, 105038, 105039, 105040, 105041, 105042, 105043, 105044, 105045, 105046) c6( ) c7(ck, T, yi, id, aW, fJ, aT, dL, eL); #c1(NUMA1) c2(NP_006176) c3(nuclear mitotic apparatus protein 1 isoform 1) c4(162557, 162558, 162559, 162560, 162561, 162562, 162563, 162564, 162565, 162566, 162567, 162568, 162569) c5(162570, 162571, 162572, 162573, 162574, 162575, 162576, 162577, 162578, 162579, 162580, 162581, 162582, 162583, 162584, 162585, 162586, 162587) c6( ) c7(ck, T, yi, id, aW, fJ, aT, dL, eL); #c1(NUMA1) c2(XP_011543366) c3(nuclear mitotic apparatus protein 1 isoform X3) c4( ) c5(342147, 342148, 342149, 342150, 342151, 342152, 342153, 342154, 342155, 342156, 342157, 342158, 342159, 342160, 342161, 342162, 342163, 342164, 342165, 342166, 342167, 342168, 342169, 342170, 342171, 342172, 342173, 342174, 342175, 342176, 342177, 342178, 342179, 342180, 342181, 342182, 342183, 342184, 342185, 342186, 342187, 342188, 342189, 342190, 342191, 342192, 342193, 342194, 342195, 342196, 342197, 342198, 342199, 342200, 342201, 342202, 342203, 342204, 342205, 342206, 342207, 342208, 342209) c6( ) c7(ck, T, yi, id, aW, fJ, aT, dL, eL); #c1(NUMA1) c2(XP_011543367) c3(nuclear mitotic apparatus protein 1 isoform X4) c4(342210) c5(342211, 342212, 342213, 342214, 342215, 342216) c6( ) c7(ck, T, yi, id, aW, fJ, aT, dL, eL); #c1 (NUMB) c2(NP_001005745) c3(protein numb homolog isoform 4) c4(22011) c5(22012, 22013, 22014, 22015, 22016, 22017, 22018, 22019, 22020, 22021, 22022) c6( ) c7(T, dD, d, af, iU, aW, aT, aT, eq, ci, lS, eA, bw, bt, bz, X); #c1(NUMB) c2(XP_011535557) c3(protein numb homolog isoform X2) c4(332646, 332647) c5(332648, 332649, 332650, 332651, 332652, 332653, 332654, 332655, 332656, 332657) c6( ) c7(T, dD, d, af, iU, aW, aT, aT, eq, ci, lS, eA, bw, bt, bz, X); #c1(NUMB) c2(XP_011535560) c3(protein numb homolog isoform X1) c4(332658, 332659, 332660) c5(332661, 332662, 332663, 332664, 332665, 332666, 332667, 332668, 332669, 332670, 332671, 332672, 332673, 332674, 332675) c6( ) c7(T, dD, d, af, iU, aW, aT, aT, eq, ci, lS, eA, bw, bt, bz, X); #c1(NUMB) c2(XP_011535562) c3(protein numb homolog isoform X3) c4(332676) c5(332677, 332678, 332679, 332680, 332681, 332682, 332683, 332684, 332685, 332686, 332687, 332688, 332689, 332690, 332691, 332692, 332693, 332694, 332695, 332696) c6( ) c7(T, dD, d, af, iU, aW, al, aT, eq, ci, lS, eA, bw, bt, bz, X); #c1(NUMB) c2(XP_011535565) c3(protein numb homolog isoform X8) c4(332697, 332698) c5(332699, 332700, 332701, 332702, 332703, 332704, 332705, 332706, 332707, 332708, 332709, 332710) c6( ) c7(T, dD, d, af, iU, aW, aT, al, eq, ci, lS, eA, bw, bt, bz, X); #c1(NUMB) c2(XP_011535566) c3 (protein numb homolog isoform X9) c4( ) c5(332711, 332712, 332713, 332714, 332715, 332716) c6( ) c7(T, dD, d, af, iU, aW, aT, al, eq, ci, lS, eA, bw, bt, bz, X); #c1(NUMBL) c2(NP_001276909) c3(numb-like protein isoform b) c4(108604) c5(108605, 108606, 108607, 108608, 108609) c6( ) c7(cm, T, eA); #c1(NUMBL) c2(NP_004747) c3(numb-like protein isoform a) c4(149239) c5(149240, 149241, 149242, 149243, 149244, 149245, 149246) c6( ) c7(cm, T, eA); #c1(NUMBL) c2(XP_011525791) c3(numb-like protein isoform X2) c4( ) c5(320197) c6( ) c7(cm, T, eA); #c1(NUP153) c2(NP_001265138) c3(nuclear pore complex protein Nup153 isoform 1) c4(97841) c5(97842, 97843, 97844, 97845, 97846, 97847) c6( ) c7(bP, aE, fR, h, lo, gn); #c1(NUP153) c2(NP_001265139) c3(nuclear pore complex protein Nup153 isoform 3) c4(97848) c5(97849, 97850, 97851, 97852, 97853, 97854) c6( ) c7(bP, aE, fR, h, lo, gn); #c1(NUP153) c2(NP_005115) c3(nuclear pore complex protein Nup153 isoform 2) c4(153017) c5(153018, 153019, 153020, 153021, 153022, 153023) c6( ) c7(bP, aE, fR, h, lo, gn); #c1(NUP155) c2(NP_001265241) c3(nuclear pore complex protein Nup155 isoform 3) c4(98032) c5(98033, 98034, 98035, 98036, 98037, 98038) c6( ) c7(h, wL, e, BD, mr); #c1(NUP155) c2(NP_004289) c3(nuclear pore complex protein Nup155 isoform 2) c4( ) c5(144882, 144883, 144884, 144885, 144886) c6( ) c7(h, wL, e, BD, mr); #c1(NUP155) c2(NP_705618) c3(nuclear pore complex protein Nup155 isoform 1) c4(246166, 246167, 246168) c5(246169, 246170, 246171, 246172, 246173, 246174, 246175, 246176, 246177, 246178, 246179, 246180, 246181) c6( ) c7(h, wL, e, BD, mr); #c1(NUP205) c2(NP_055950) c3(nuclear pore complex protein Nup205) c4(184624, 184625) c5(184626, 184627, 184628, 184629, 184630, 184631, 184632, 184633, 184634, 184635, 184636, 184637) c6( ) c7(bz, bi, bdM); #c1(NUP210) c2(NP_079199) c3(nuclear pore membrane glycoprotein 210 precursor) c4(217459) c5(217460, 217461, 217462, 217463, 217464, 217465) c6( ) c7(q, ac, bc, r); #c1(NUP214) c2(NP_005076) c3(nuclear pore complex protein Nup214) c4(152653) c5(152654, 152655, 152656, 152657, 152658, 152659, 152660, 152661, 152662, 152663, 152664, 152665) c6( ) c7(bM, T, Dw, Va, if, jx, jh, aA, Bm, mS, bB, fd, ci, nA, qN, hF, q, eA, hX, cc, mW, ds, cmp, dV, aT, cU, h, bK, K, jj, H, mV, mY, pk, de, bF, mU, B, iU, aW, Fm, Y, yx, apn, ff, r, kD, lo, Kf, aF, beP, en, YZ, t, bz, mZ, f, ek, mX, ck, dR, cm, y, bb, VJ, c, eM, bE, Om, bP, iT, AH, aiM, cj, m, YH, ph, bgi, bt, kS, mT, ao, L); #c1(NUP214) c2(XP_005272273) c3(nuclear pore complex protein Nup214 isoform X1) c4(283970, 283971) c5(283972, 283973, 283974, 283975, 283976, 283977, 283978, 283979, 283980, 283981, 283982, 283983) c6( ) c7(bM, T, Dw, Va, if, jx, jh, aA, Bm, mS, bB, fd, ci, nA, qN, hF, q, eA, hX, cc, mW, ds, cmp, dV, aT, cU, h, bK, K, jj, H, mV, mY, pk, de, bF, mU, B, iU, aW, Fm, Y, yx, apn, ff, r, kD, lo, Kf, aF, beP, en, YZ, t, bz, mZ, f, ek, mX, ck, dR, cm, y, bb, VJ, c, eM, bE, Om, bP, iT, AH, aiM, cj, m, YH, ph, bgi, bt, kS, mT, ao, L); #c1(NUP214) c2(XP_005272275) c3 (nuclear pore complex protein Nup214 isoform X5) c4(283984) c5( ) c6( ) c7(bM, T, Dw, Va, if, jx, jh, aA, Bm, mS, bB, fd, ci, nA, qN, hF, q, eA, hX, cc, mW, ds, cmp, dV, aT, cU, h, bK, K, jj, H, mV, mY, pk, de, bF, mU, B, iU, aW, Fm, Y, yx, apn, ff, r, kD, lo, Kf, aF, beP, en, YZ, t, bz, mZ, f, ek, mX, ck, dR, cm, y, bb, VJ, c, eM, bE, Om, bP, iT, AH, aiM, cj, m, YH, ph, bgi, bt, kS, mT, ao, L); #c1(NUP214) c2(XP_005272276) c3(nuclear pore complex protein Nup214 isoform X6) c4(283985) c5( ) c6( ) c7(bM, T, Dw, Va, if, jx, jh, aA, Bm, mS, bB, fd, ci, nA, qN, hF, q, eA, hX, cc, mW, ds, cmp, dV, aT, cU, h, bK, K, jj, H, mV, mY, pk, de, bF, mU, B, iU, aW, Fm, Y, yx, apn, ff, r, kD, lo, Kf, aF, beP, en, YZ, t, bz, mZ, f, ek, mX, ck, dR, cm, y, bb, VJ, c, eM, bE, Om, bP, iT, AH, aiM, cj, m, YH, ph, bgi, bt, kS, mT, ao, L); #c1(NUP214) c2(XP_006717355) c3(nuclear pore complex protein Nup214 isoform X3) c4(290966) c5(290967, 290968, 290969, 290970, 290971, 290972) c6( ) c7(bM, T, Dw, Va, if, jx, jh, aA, Bm, mS, bB, fd, ci, nA, qN, hF, q, eA, hX, cc, mW, ds, cmp, dV, aT, cU, h, bK, K, jj, H, mV, mY, pk, de, bF, mU, B, iU, aW, Fm, Y, yx, apn, ff, r, kD, lo, Kf, aF, beP, en, YZ, t, bz, mZ, f, ek, mX, ck, dR, cm, y, bb, VJ, c, eM, bE, Om, bP, iT, AH, aiM, cj, m, YH, ph, bgi, bt, kS, mT, ao, L); #c1(NUP214) c2(XP_011517355) c3(nuclear pore complex protein Nup214 isoform X4) c4( ) c5(311089) c6( ) c7(bM, T, Dw, Va, if, jx, jh, aA, Bm, mS, bB, fd, ci, nA, qN, hF, q, eA, hX, cc, mW, ds, cmp, dV, aT, cU, h, bK, K, jj, H, mV, mY, pk, de, bF, mU, B, iU, aW, Fm, Y, yx, apn, ff, r, kD, lo, Kf, aF, beP, en, YZ, t, bz, mZ, f, ek, mX, ck, dR, cm, y, bb, VJ, c, eM, bE, Om, bP, iT, AH, aiM, cj, m, YH, ph, bgi, bt, kS, mT, ao, L); #c1(NUP35) c2(NP_001274513) c3(nucleoporin NUP53 isoform b) c4(106591) c5(106592, 106593, 106594, 106595, 106596) c6( ) c7(aw); #c1(NUP35) c2(NP_001274514) c3(nucleoporin NUP53 isoform c) c4( ) c5(106597, 106598, 106599, 106600, 106601) c6( ) c7(aw); #c1(NUP35) c2(NP_612142) c3(nucleoporin NUP53 isoform a) c4(236047, 236048) c5(236049, 236050, 236051, 236052, 236053, 236054, 236055, 236056) c6( ) c7(aw); #c1(NUP37) c2(NP_076962) c3(nucleoporin Nup37) c4(214478, 214479) c5(214480, 214481, 214482, 214483, 214484, 214485, 214486, 214487, 214488, 214489, 214490, 214491, 214492, 214493, 214494, 214495) c6( ) c7(Pg); #c1(NUP43) c2(NP_942590) c3(nucleoporin Nup43) c4(260565) c5(260566, 260567, 260568, 260569, 260570, 260571, 260572, 260573, 260574, 260575) c6( ) c7(h, T, yw, kB); #c1(NUP43) c2(XP_005267019) c3(nucleoporin Nup43 isoform X4) c4( ) c5(280626, 280627, 280628, 280629, 280630, 280631) c6( ) c7(h, T, yw, kB); #c1(NUP43) c2(XP_011534100) c3(nucleoporin Nup43 isoform X1) c4(330354) c5( ) c6( ) c7(h, T, yw, KB); #c1(NUP50) c2(XP_005261370) c3(nuclear pore complex protein Nup50 isoform X2) c4( ) c5(277303, 277304, 277305, 277306, 277307, 277308, 277309, 277310, 277311, 277312, 277313, 277314, 277315, 277316) c6( ) c7(j); #c1(NUP50) c2(XP_011528135) c3(nuclear pore complex protein Nup50 isoform X1) c4(323219, 323220, 323221, 323222, 323223, 323224) c5(323225, 323226, 323227, 323228, 323229, 323230, 323231, 323232, 323233, 323234, 323235, 323236, 323237, 323238, 323239, 323240, 323241, 323242, 323243, 323244, 323245, 323246, 323247, 323248, 323249, 323250, 323251, 323252, 323253, 323254, 323255, 323256) c6( ) c7(j); #c1(NUP62) c2(NP_001180286) c3(nuclear pore glycoprotein p62) c4(73715, 73716, 73717) c5(73718, 73719, 73720, 73721, 73722, 73723, 73724, 73725, 73726, 73727, 73728, 73729, 73730, 73731, 73732, 73733, 73734, 73735, 73736, 73737, 73738, 73739, 73740, 73741, 73742, 73743) c6( ) c7(bM, T, FR, if, GC, To, v, BF, ci, q, ad, Kx, jz, zw, WY, aT, h, K, Wr, ae, af, d, aW, ff, az, alR, r, aq, aaE, kp, en, g, cmq, bb, kP, c, eM, U, Kj, bdR, bi, xg, B); #c1(NUP85) c2(NP_001290205) c3(nuclear pore complex protein Nup85 isoform 2) c4(115172) c5(115173, 115174, 115175, 115176, 115177) c6( ) c7(By, cd); #c1(NUP85) c2(NP_079120) c3(nuclear pore complex protein Nup85 isoform 1) c4(217/75, 217176) c5(217177, 217178, 217179, 217180, 217181, 217182, 217183, 217184) c6( ) c7(By, cd); #c1(NUP85) c2(XP_005257749) c3(nuclear pore complex protein Nup85 isoform X5) c4( ) c5(275359, 275360, 275361, 275362, 275363, 275364) c6( ) c7(By, cd); #c1(NUP85) c2(XP_011523569) c3(nuclear pore complex protein Nup85 isoform X2) c4(318065) c5(318066, 318067, 318068, 318069, 318070) c6( ) c7(By, cd); #c1(NUP85) c2(XP_011523570) c3(nuclear pore complex protein Nup85 isoform X4) c4(318071) c5(318072, 318073, 318074, 318075, 318076, 318077) c6( ) c7(By, cd); #c1(NUP88) c2(NP_002523) c3(nuclear pore complex protein Nup88) c4(129517) c5(129518, 129519, 129520, 129521, 129522, 129523, 129524, 129525, 129526, 129527) c6( ) c7(T, aW, r, h, aT, q); #c1(NUP88) c2(XP_005256716) c3(nuclear pore complex protein Nup88 isoform X1) c4( ) c5(274303) c6( ) c7(T, aW, r, h, al, q); #c1(NUP88) c2(XP_011522195) c3(nuclear pore complex protein Nup88 isoform X2) c4(316819) c5(316820, 316821, 316822, 316823, 316824) c6( ) c7(T, aW, r, h, aT, q); #c1(NUP93) c2(XP_005256320) c3(nuclear pore complex protein Nup93 isoform X1) c4(274096, 274097) c5(274098, 274099, 274100, 274101, 274102, 274103, 274104, 274105, 274106, 274107, 274108, 274109, 274110, 274111, 274112, 274113) c6( ) c7(fq); #c1(NUP98) c2(NP_005378) c3(nuclear pore complex protein Nup98-Nup96 isoform 3) c4( ) c5(155576, 155577, 155578, 155579, 155580, 155581) c6( ) c7(T, dA, gp, al, bO, n, ci, hF, br, x, fX, pp, dE, h, dz, hn, bK, he, pq, d, iU, et, gK, eZ, aq, as, hf, bw, bz, ck, c, cA, eM, iT, bt, B, EB); #c1(NUP98) c2(NP_057404) c3(nuclear pore complex protein Nup98-Nup96 isoform 1) c4(190567, 190568) c5(190569, 190570, 190571, 190572, 190573, 190574) c6( ) c7(T, dA, gp, al, bO, n, ci, hF, br, x, fX, pp, dE, h, dz, hn, bK, he, pq, d, iU, et, gK, eZ, aq, as, hf, bw, bz, ck, c, cA, eM, iT, bt, B, EB); #c1(NUP98) c2(NP_624357) c3(nuclear pore complex protein Nup98-Nup96 isoform 2) c4( ) c5(238153, 238154, 238155, 238156, 238157, 238158) c6( ) c7(T, dA, gp, al, bO, n, ci, hF, br, x, fX, pp, dE, h, dz, hn, bK, he, pq, d, iU, et, gK, eZ, aq, as, hf, bw, bz, ck, c, cA, eM, iT, bt, B, EB); #c1(NUP98) c2(NP_624358) c3(nuclear pore complex protein Nup98-Nup96 isoform 4) c4(238159, 238160) c5(238161, 238162, 238163, 238164, 238165, 238166, 238167, 238168, 238169, 238170, 238171, 238172) c6( ) c7(T, dA, gp, al, bO, n, ci, hF, br, x, fX, pp, dE, h, dz, hn, bK, he, pq, d, iU, et, gK, eZ, aq, as, hf, bw, bz, ck, c, cA, eM, iT, bt, B, EB); #c1(NUP98) c2(XP_011518437) c3(nuclear pore complex protein Nup98-Nup96 isoform X1) c4(312502) c5( ) c6( ) c7(T, dA, gp, al, bO, n, ci, hF, br, x, fX, pp, dE, h, dz, hn, bK, he, pq, d, iU, et, gK, eZ, aq, as, hf, bw, bz, ck, c, cA, eM, iT, bt, B, EB); #c1(NUPL2) c2(NP_031368) c3(nucleoporin-like protein 2) c4(171966) c5(171967, 171968, 171969, 171970, 171971, (71972) c6( ) c7(yy); #c1(NUPR1) c2(NP_001035948) c3(nuclear protein 1 isoform a) c4(33515) c5(33516, 33517, 33518, 33519, 33520, 33521) c6( ) c7(bL, T, aC, v, ci, iJ, q, ad, aE, Db, aT, dL, Fx, K, ae, eL, s, aW, r, akh, kp, qJ, ar, aQd, nB, mf, du); #c1(NUPR1) c2(NP_036517) c3(nuclear protein 1 isoform b) c4(174213) c5(174214, 174215, 174216, 174217, 174218, 174219) c6( ) c7(bL, T, aC, v, ci, iJ, q, ad, aE, Db, aT, dL, Fx, K, ae, eL, s, aW, r, akh, kp, qJ, ar, aQd, nB, mf, du); #c1(NUS1) c2(NP_612468) c3(nogo-B receptor) c4(236607) c5(236608, 236609, 236610, 236611, 236612, 236613) c6( ) c7(T, Y); #c1(NUSAP1) c2(NP_001230071) c3(nucleolar and spindle-associated protein 1 isoform 4) c4(85868) c5(85869, 85870, 85871, 85872, 85873, 85874) c6( ) c7(qt, aMb, n, ey, ae, ad); #c1(NUSAP1) c2(NP_001230072) c3(nucleolar and spindle-associated protein 1 isoform 5) c4(85875) c5(85876, 85877, 85878, 85879, 85880, 85881) c6( ) c7(qt, aMb, n, ey, ae, ad); #c1(NUSAP1) c2(NP_001288065) c3(nucleolar and spindle-associated protein 1 isoform 7) c4(113477) c5(113478, 113479, 113480, 113481, 113482, 113483) c6( ) c7(qt, aMb, n, ey, ae, ad); #c1(NUSAP1) c2(NP_057443) c3(nucleolar and spindle-associated protein 1 isoform 1) c4(190916) c5(190917, 190918, 190919, 190920, 190921, 190922) c6( ) c7(qt, aMb, n, ey, ae, ad); #c1(NUSAP1) c2(NP_060924) c3(nucleolar and spindle-associated protein 1 isoform 2) c4(198700) c5(198701, 198702, 198703, 198704, 198705, 198706) c6( ) c7(qt, aMb, n, ey, ae, ad); #c1(NUSAP1) c2(XP_006720626) c3(nucleolar and spindle-associated protein 1 isoform X8) c4(293589) c5(293590, 293591, 293592, 293593, 293594, 293595) c6( ) c7(qt, aMb, n, ey, ae, ad); #c1(NUTF2) c2(XP_005255828) c3(nuclear transport factor 2 isoform X1) c4(273879) c5(273880, 273881, 273882, 273883, 273884, 273885) c6( ) c7(h); #c1(NUTM1) c2(NP_001271221) c3(NUT family member 1 isoform 1) c4(103650) c5(103651, 103652, 103653, 103654, 103655, 103656, 103657, 103658) c6( ) c7(aJP, T, Jg, vT, ff, awr, ci, en, aws, Le, pd, avU); #c1(NUTM1) c2(NP_786883) c3(NUT family member 1 isoform 3) c4(251908) c5( ) c6( ) c7(aJP, T, Jg, vT, ff, awr, ci, en, aws, Le, pd, avU); #c1(NUTM1) c2(XP_011519731) c3(NUT family member 1 isoform X1) c4(314395) c5(314396, 314397, 314398, 314399, 314400, 314401, 314402, 314403, 314404, 314405) c6( ) c7(aJP, T, Jg, vT, ff, awr, ci, en, aws, Le, pd, avU); #c1(NUTM2A) c2(XP_011538424) c3(NUT family member 2A isoform X1) c4(336107, 336108, 336109, 336110, 336111, 336112, 336113, 336114) c5(336115, 336116, 336117, 336118, 336119, 336120, 336121, 336122, 336123, 336124, 336125, 336126, 336127, 336128, 336129, 336130, 336131, 336132, 336133, 336134, 336135, 336136, 336137, 336138, 336139, 336140, 336141, 336142, 336143, 336144, 336145, 336146, 336147, 336148, 336149, 336150, 336151, 336152, 336153, 336154, 336155, 336156, 336157, 336158, 336159, 336160, 336161, 336162) c6( ) c7(Th); #c1(NUTM2A) c2(XP_011538425) c3(NUT family member 2A isoform X2) c4(336163, 336164, 336165, 336166) c5(336167, 336168, 336169, 336170, 336171, 336172, 336173, 336174, 336175, 336176, 336177, 336178, 336179, 336180, 336181, 336182, 336183, 336184, 336185, 336186, 336187, 336188, 336189, 336190) c6( ) c7(Th); #c1(NUTM2B) c2(XP_005270192) c3(NUT family member 28 isoform X1) c4(283222, 283223, 283224) c5(283225, 283226, 283227, 283228, 283229, 283230, 283231, 283232, 283233, 283234, 283235, 283236, 283237, 283238, 283239, 283240, 283241, 283242) c6( ) c7(Th); #c1(NVL) c2(NP_001230075) c3(nuclear valosin-containing protein-like isoform 3) c4( ) c5(85882, 85883, 85884, 85885, 85886, 85887) c6( ) c7(cd, ac); #c1(NVL) c2(NP_001230076) c3(nuclear valosin-containing protein-like isoform 4) c4(85888) c5(85889, 85890, 85891, 85892, 85893, 85894) c6( ) c7(cd, ac); #c1(NVL) c2(NP_002524) c3(nuclear valosin-containing protein-like isoform 1) c4(129528, 129529, 129530) c5(129531, 129532, 129533, 129534, 129535, 129536, 129537, 129538, 129539, 129540, 129541, 129542, 129543, 129544, 129545, 129546, 129547, 129548) c6( ) c7(cd, ac); #c1(NVL) c2(XP_011542498) c3(nuclear valosin-containing protein-like isoform X1) c4(341051) c5( ) c6( ) c7(cd, ac); #c1(NWD1) c2(NP_001007526) c3(NACHT and WD repeat domain-containing protein 1 isoform 1) c4(22749) c5(22750, 22751, 22752, 22753, 22754, 22755, 22756, 22757, 22758, 22759) c6( ) c7(ae, ad); #c1(NWD1) c2(NP_001277284) c3(NACHT and WD repeat domain-containing protein 1 isoform 2) c4(109323, 109324) c5(109325, 109326, 109327, 109328, 109329, 109330, 109331, 109332, 109333, 109334, 109335, 109336, 109337, 109338, 109339) c6( ) c7(ae, ad); #c1(NWD1) c2(XP_011526233) c3(NACHT and WD repeat domain-containing protein 1 isoform X2) c4( ) c5(320492) c6( ) c7(ae, ad); #c1(NXF1) c2(NP_001074960) c3(nuclear RNA export factor 1 isoform 2) c4(37142) c5(37143, 37144, 37145, 37146, 37147, 37148, 37149, 37150, 37151, 37152) c6( ) c7(oO, T, bvt, if, jx, aA, eb, n, cL, fr, bvx, ci, qN, V, eW, o, hX, jF, av, F, h, K, hu, hH, aa, uQ, A, mR, dH, Y, KS, cr, eZ, afq, lo, aF, xH, hp, iV, eE, iK, bz, ig, bvy, lF, y, qR, hv); #c1(NXF1) c2(NP_006353) c3(nuclear RNA export factor 1 isoform 1) c4(164277) c5(164278, 164279, 164280, 164281, 164282, 164283, 164284, 164285, 164286, 164287) c6( ) c7(oO, T, bvt, if, jx, aA, eb, n, cL, fr, bvx, ci, qN, V, eW, o, hX, jF, av, F, h, K, hu, hH, aa, uQ, A, mR, dH, Y, KS, cr, eZ, afq, lo, aF, xH, hp, iV, eE, iK, bz, ig, bvy, lF, y, qR, hv); #c1(NXF2B) c2(NP_001093156) c3(nuclear RNA export factor 2) c4(39841, 39842) c5(39843, 39844, 39845, 39846, 39847, 39848, 39849, 39850, 39851, 39852, 39853, 39854, 39855, 39856, 39857, 39858, 39859, 39860, 39861, 39862, 39863, 39864, 39865, 39866, 39867, 39868, 39869, 39870, 39871, 39872, 39873, 39874, 39875, 39876, 39877, 39878, 39879, 39880, 39881, 39882, 39883, 39884, 39885, 39886, 39887, 39888, 39889, 39890, 39891, 39892, 39893, 39894, 39895, 39896) c6( ) c7(wu, pt, wm, cj); #c1(NXF3) c2(NP_071335) c3(nuclear RNA export factor 3) c4(211065, 211066, 211067, 211068) c5(211069, 211070, 211071, 211072, 211073, 211074, 211075, 211076, 211077, 211078, 211079, 211080, 211081, 211082, 211083, 211084, 211085, 211086, 211087, 211088, 211089, 211090, 211091, 211092) c6( ) c7(Na); #c1(NXF5) c2(NP_116564) c3(nuclear RNA export factor 5) c4(226192) c5(226193, 226194, 226195, 226196, 226197, 226198, 226199, 226200, 226201, 226202, 226203, 226204, 226205, 226206, 226207, 226208, 226209) c6( ) c7(nf, il, nA); #c1(NXF5) c2(XP_011529294) c3(nuclear RNA export factor 5 isoform X3) c4(324726) c5( ) c6( ) c7(nf, il, nA); #c1(NXN) c2(NP_001192248) c3(nucleoredoxin isoform 2) c4(82149) c5(82150, 82151, 82152, 82153, 82154, 82155, 82156, 82157, 82158, 82159) c6( ) c7(bK); #c1(NXN) c2(NP_071908) c3(nucleoredoxin isoform 1) c4(212224) c5(212225, 212226, 212227, 212228, 212229, 212230) c6( ) c7(bK); #c1(NXN) c2(XP_005256813) c3(nucleoredoxin isoform X1) c4(274523) c5(274524, 274525, 274526, 274527, 274528, 274529) c6( ) c7(bK); #c1(NXNL1) c2(NP_612463) c3(nucleoredoxin-like protein 1) c4(236571) c5(236572, 236573, 236574, 236575, 236576, 236577) c6( ) c7(lR, nC, K, Tp); #c1(NXNL2) c2(NP_001155097) c3(nucleoredoxin-like protein 2 isoform 1) c4(61440) c5(61441, 61442, 61443, 61444, 61445, 61446) c6( ) c7(cd); #c1(NXNL2) c2(NP_660326) c3(nucleoredoxin-like protein 2 isoform 2) c4(240934) c5(240935, 240936, 240937, 240938, 240939, 240940) c6( ) c7(cd); #c1(NXPE1) c2(NP_689528) c3(NXPE family member 1) c4( ) c5(242992, 242993, 242994, 242995, 242996, 242997, 242998) c6(242999, 243000) c7(aw, iV); #c1(NXPE1) c2(XP_011540901) c3(NXPE family member 1 isoform X1) c4(339438, 339439) c5(339440, 339441, 339442, 339443, 339444, 339445, 339446, 339447, 339448) c6(339449, 339450) c7(aw, iV); #c1(NXPE2) c2(NP_872301) c3(NXPE family member 2) c4(256053, 256054) c5(256055, 256056, 256057, 256058, 256059, 256060, 256061, 256062, 256063, 256064, 256065, 256066) c6( ) c7(oe, sj); #c1(NXPE4) c2(NP_001071107) c3(NXPE family member 4 isoform 1 precursor) c4(35281, 35282) c5(35283, 35284, 35285, 35286, 35287, 35288, 35289, 35290, 35291) c6(35292, 35293) c7(aw); #c1(NXPE4) c2(NP_060148) c3(NXPE family member 4 isoform 2) c4( ) c5(194487, 194488, 194489, 194490, 194491, 194492, 194493) c6(194494, 194495) c7(aw); #c1(NXPH1) c2(NP_689958) c3(neurexophilin-1 precursor) c4(244545, 244546, 244547, 244548) c5(244549, 244550, 244551, 244552, 244553, 244554, 244555, 244556, 244557, 244558, 244559, 244560, 244561, 244562, 244563, 244564, 244565, 244566, 244567, 244568, 244569, 244570, 244571, 244572, 244573, 244574, 244575, 244576, 244577, 244578, 244579) c6(244580, 244581, 244582, 244583, 244584, 244585, 244586, 244587) c7(blk, pH, bM, aC, agJ, eR, oe, cm, aj, vm, dy, nA, aY, vN, ad); #c1(NXPH1) c2(XP_011513634) c3(neurexophilin-1 isoform X1) c4( ) c5(305944) c6( ) c7(blk, pH, bM, aC, agJ, eR, oe, cm, aj, vm, dy, nA, aY, vN, ad); #c1 (NXPH2) c2(NP_009157) c3 (neurexophilin-2 precursor) c4(171188) c5(171189, 171190, 171191, 171192, 171193, 171194, 171195, 171196) c6(171197, 171198) c7(gU, EQ, cm, m, t, cd); #c1(NXT1) c2(XP_011527532) c3 (NTF2-related export protein 1 isoform X1) c4(322340) c5(322341, 322342, 322343, 322344, 322345, 322346) c6( ) c7(T, aC, fM, id, ht, jx, jh, aA, fd, j, ci, hF, Fc, q, ad, eA, ip, x, av, iu, fX, ib, nv, kH, aT, h, hn, dL, jj, XZ, hu, hS, pq, ae, eL, Zm, d, je, s, aix, iU, aW, Y, ff, r, aF, en, cl, as, t, ik, bw, iP, ck, qJ, iM, bP, eM, aJm, dB, iT, cj, pg, k, bt, B, vi); #c1(NXT2) c2(NP_001229546) c3(NTF2-related export protein 2 isoform 2) c4( ) c5(84624, 84625, 84626, 84627, 84628) c6( ) c7(aYJ, atf, ey, nA, UK, axC); #c1(NXT2) c2(NP_001229547) c3(NTF2-related export protein 2 isoform 3) c4( ) c5(84629, 84630, 84631, 84632, 84633, 84634, 84635, 84636) c6( ) c7(aYJ, atf, ey, nA, UK, axC); #c1(NXT2) c2(NP_061168) c3(NTF2-related export protein 2 isoform 1) c4(199707, 199708, 199709) c5(199710, 199711, 199712, 199713, 199714, 199715, 199716, 199717) c6( ) c7(aYJ, atf, ey, nA, UK, axC); #c1(NXT2) c2(XP_011529291) c3(NTF2-related export protein 2 isoform X1) c4( ) c5(324725) c6( ) c7(aYJ, atf, ey, nA, UK, axC); #c1(NYAP2) c2(NP_065915) c3(neuronal tyrosine-phosphorylated phosphoinositide-3-kinase adapter 2) c4(206215, 206216) c5(206217, 206218, 206219, 206220, 206221, 206222, 206223, 206224, 206225, 206226, 206227, 206228) c6( ) c7(bL, ayB, v, j, an); #c1(NYX) c2(NP_072089) c3(nyctalopin precursor) c4(212652) c5(212653, 212654, 212655, 212656, 212657, 212658, 212659, 212660, 212661, 212662) c6( ) c7(ck, cZ, lR, Be, ayA, cmr, OL, dd, HU, ayj, nC); #c1(DARD1) c2(NP_659500) c3(O-acetyl-ADP-ribose deacetylase 1) c4(240221, 240222, 240223) c5(240224, 240225, 240226, 240227, 240228, 240229, 240230, 240231, 240232, 240233, 240234, 240235, 240236, 240237, 240238, 240239, 240240, 240241, 240242, 240243, 240244, 240245, 240246, 240247, 240248, 240249, 240250, 240251, 240252, 240253) c6( ) c7(U); #c1(OAS1) c2(NP_001027581) c3(2′-5′-oligoadenylate synthase 1 isoform 3) c4(30308) c5(30309, 30310, 30311, 30312, 30313, 30314) c6( ) c7(bL, fR, aT, jx, jE, V, ad, hX, F, ds, oW, h, cfr, ae, A, Y, wc, eZ, lo, t, AH, aiM, aEU); #c1(DAS1) c2(NP_002525) c3(2′-5′-oligoadenylate synthase 1 isoform 2) c4(129549) c5(129550, 129551, 129552, 129553, 129554, 129555) c6( ) c7(bL, fR, al, jx, jE, V, ad, hX, F, ds, oW, h, cfr, ae, A, Y, wc, eZ, lo, t, AH, aiM, aEU); #c1(DAS1) c2(NP_058132) c3(2′-5′-oligoadenylate synthase 1 isoform 1) c4(192653) c5(192654, 192655, 192656, 192657, 192658, 192659) c6( ) c7(bL, fR, al, jx, jE, V, ad, hX, F, ds, oW, h, cfr, ae, A, Y, wc, eZ, lo, t, AH, aiM, aEU); #c1(DAS1) c2(XP_011536716) c3(2′-5′-oligoadenylate synthase 1 isoform X3) c4(334310) c5(334311, 334312, 334313, 334314, 334315, 334316) c6( ) c7(bL, fR, aT, jx, jE, V, ad, hX, F, ds, oW, h, cfr, ae, A, Y, wc, eZ, lo, t, AH, aiM, aEU); #c1(DAS2) c2(NP_001027903) c3(2′-5′-oligoadenylate synthase 2 isoform 3) c4(30315) c5(30316, 30317, 30318, 30319, 30320, 30321) c6( ) c7(ke, A, ds, AH, jE, CQ, ae, ad, hX); #c1(DAS2) c2(NP_002526) c3(2′-5′-oligoadenylate synthase 2 isoform 2) c4(129556) c5(129557, 129558, 129559, 129560, 129561, 129562) c6( ) c7(ke, A, ds, AH, jE, CQ, ae, ad, hX); #c1(DAS2) c2(NP_058197) c3(2′-5′-oligoadenylate synthase 2 isoform 1) c4(192660) c5(192661, 192662, 192663, 192664, 192665, 192666) c6( ) c7(ke, A, ds, AH, jE, CQ, ae, ad, hX); #c1(DAS3) c2(NP_006178) c3(2′-5′-oligoadenylate synthase 3) c4(162608, 162609) c5(162610, 162611, 162612, 162613, 162614, 162615, 162616, 162617, 162618, 162619, 162620, 162621) c6( ) c7(ke, A, iM, je, ds, AH, jh, kH, ahC, Zn, cz, bB, Da, jE, qN, fd, t, ae, ad, hX); #c1(DASL) c2(NP_003724) c3(2′-5′-oligoadenylate synthase-like protein isoform a) c4(140458) c5(140459, 140460, 140461, 140462, 140463, 140464) c6( ) c7(bL, F, ds, aiM, eZ, jE); #c1(DASL) c2(NP_937856) c3(2′-5′-oligoadenylate synthase-like protein isoform b) c4(259308) c5(259309, 259310, 259311, 259312, 259313, 259314) c6( ) c7(bL, F, ds, aiM, eZ, jE); #c1(DAT) c2(NP_001165285) c3(ornithine aminotransferase, mitochondrial isoform 2) c4( ) c5(67586, 67587, 67588, 67589, 67590) c6( ) c7(cms, cmu, cmt, bS, v, bpT, iJ, afR, aah, vH, aXd, rL, A, zC, dr, bDw, bP, U, wz, nw, be, oQ); #c1(DAT) c2(XP_011538136) c3(ornithine aminotransferase, mitochondrial isoform X1) c4(335719, 335720) c5(335721, 335722, 335723, 335724, 335725, 335726, 335727) c6( ) c7(cms, cmu, cmt, bS, v, bpT, iJ, afR, aah, vH, aXd, rL, A, zC, dr, bDw, bP, U, wz, nw, be, oQ); #c1(DAZ1) c2(NP_001287949) c3(ornithine decarboxylase antizyme 1 isoform 2) c4(113264) c5(113265) c6( ) c7(T, y, dY, eM, e, eZ, cm, iy, bS, j, fL, m, t, ae, ad); #c1(DAZ1) c2(NP_004143) c3(ornithine decarboxylase antizyme 1 isoform 1) c4( ) c5(143857, 143858, 143859, 143860, 143861, 143862) c6( ) c7(T, y, dY, eM, e, eZ, cm, iy, bS, j, fL, m, t, ae, ad); #c1(OAZ2) c2(NP_002528) c3(ornithine decarboxylase antizyme 2 isoform 1) c4(129591) c5(129592, 129593, 129594, 129595, 129596, 129597) c6( ) c7(cm); #c1(OBFC1) c2(XP_006718039) c3(CST complex subunit STN1 isoform X1) c4(291465) c5(291466, 291467, 291468, 291469, 291470, 291471, 291472, 291473, 291474, 291475) c6( ) c7(LB, BO, e, r, q, op); #c1(OBP2A) c2(NP_001280118) c3(odorant-binding protein 2a isoform gamma precursor) c4(111325) c5(111326, 111327, 111328, 111329, 111330, 111331, 111332) c6( ) c7(ae, T, av, eM, bE, oV, PX, X, r, aA, bB, lo, aF, fr, m, q, t, ik, bz, B, ad); #c1(DBP2A) c2(NP_001280122) c3(odorant-binding protein Za isoform delta precursor) c4(111355, 111356) c5(111357, 111358, 111359, 111360, 111361, 111362, 111363, 111364, 111365, 111366) c6( ) c7(ae, T, av, eM, bE, oV, PX, X, r, aA, bB, lo, aF, fr, m, q, t, ik, bz, B, ad); #c1(OBP2A) c2(NP_055397) c3(odorant-binding protein 2a isoform alpha precursor) c4(180768) c5(180769, 180770, 180771, 180772, 180773, 180774, 180775, 180776, 180777, 180778) c6( ) c7(ae, T, av, eM, bE, oV, PX, X, r, aA, bB, lo, aF, fr, m, q, t, iK, bz, B, ad); #c1(OBSCN) c2(NP_001092093) c3(obscurin isoform b) c4(39324) c5(39325, 39326, 39327, 39328, 39329, 39330) c6( ) c7(T, W, hW, mx, r, vc, fb); #c1(OBSCN) c2(NP_001258152) c3(obscurin isoform lC) c4(95991) c5( ) c6( ) c7(T, W, hW, mx, r, vc, fb); #c1(OBSCN) c2(NP_443075) c3(obscurin isoform a) c4(229263) c5(229264, 229265, 229266, 229267, 229268, 229269, 229270, 229271, 229272, 229273, 229274, 229275) c6( ) c7(T, W, hW, mx, r, vc, fb); #c1(OBSCN) c2(XP_005273344) c3(obscurin isoform X1) c4( ) c5(284480, 284481, 284482, 284483, 284484, 284485) c6( ) c7(T, W, hW, mx, r, vc, fb); #c1(OBSCN) c2(XP_005273345) c3(obscurin isoform X2) c4( ) c5(284486, 284487, 284488, 284489, 284490, 284491) c6( ) c7(T, W, hW, mx, r, vc, fb); #c1(OBSCN) c2(XP_005273348) c3(obscurin isoform X5) c4( ) c5(284492) c6( ) c7(T, W, hW, mx, r, vc, fb); #c1(OBSCN) c2(XP_006711886) c3(obscurin isoform X23) c4(286279) c5(286280, 286281, 286282, 286283, 286284) c6( ) c7(T, W, hW, mx, r, vc, fb); #c1(OBSCN) c2(XP_006711890) c3(obscurin isoform X38) c4(286285) c5( ) c6( ) c7(T, W, hW, mx, r, vc, fb); #c1(OBSCN) c2(XP_006711891) c3(obscurin isoform X40) c4(286286) c5(286287, 286288, 286289, 286290, 286291, 286292, 286293) c6( ) c7(T, W, hW, mx, r, vc, fb); #c1 (OBSCN) c2(XP_011542599) c3(obscurin isoform X33) c4( ) c5(341166, 341167, 341168, 341169, 341170, 341171) c6( ) c7(T, W, hW, mx, r, vc, fb); #c1(OBSCN) c2(XP_011542600) c3(obscurin isoform X39) c4(34/172, 341173) c5( ) c6( ) c7(T, W, hW, mx, r, vc, fb); #c1(OBSL1) c2(NP_001166879) c3(obscurin-like protein 1 isoform 3 precursor) c4(68576) c5(68577, 68578, 68579, 68580, 68581, 68582) c6( ) c7(aCo, aCm, oj, km, cmv); #c1(OBSL1) c2(NP_056126) c3(obscurin-like protein 1 isoform 1 precursor) c4(185801) c5(185802, 185803, 185804, 185805, 185806, 185807) c6( ) c7(aCo, aCm, oj, km, cmv); #c1(OBSL1) c2(XP_005246481) c3(obscurin-like protein 1 isoform X8) c4( ) c5(267224, 267225, 267226, 267227, 267228, 267229) c6( ) c7(aCo, aCm, oj, km, cmv); #c1(OBSL1) c2(XP_011509161) c3(obscurin-like protein 1 isoform X6) c4(299792) c5( ) c6( ) c7(aCo, aCm, oj, km, cmv); #c1(OBSL1) c2(XP_011509168) c3(obscurin-like protein 1 isoform X13) c4(299793) c5(299794, 299795, 299796, 299797, 299798, 299799) c6( ) c7(aCo, aCm, oj, km, cmv); #c1(OC90) c2(NP_001073868) c3(otoconin-90 precursor) c4(36182, 36183, 36184) c5(36185, 36186, 36187, 36188, 36189, 36190, 36191, 36192, 36193, 36194, 36195, 36196, 36197, 36198, 36199, 36200, 36201, 36202, 36203, 36204) c6(36205, 36206) c7(Hn, aRa); #c1(OCA2) c2(NP_000266) c3(P protein isoform 1) c4(5939) c5( ) c6( ) c7(bL, sc, T, yk, iW, fr, bHA, q, rc, hX, cmw, av, hd, afQ, aT, SH, cmx, dL, afu, d, lR, aW, Y, ff, ih, apn, XC, eZ, lo, CQ, en, bHy, t, cm, UZ, dr, sS, RB, Dy, eM, nB, hk, agl, lF, cj, gZ, at, m, aPG, aMk); #c1(OCA2) c2(NP_001287913) c3(P protein isoform 2) c4(113198) c5( ) c6( ) c7(bL, sc, T, yk, iW, fr, bHA, q, rc, hX, cmw, av, hd, afQ, aT, SH, cmx, dL, afu, d, lR, aW, Y, ff, ih, apn, XC, eZ, lo, CQ, en, bHy, t, cm, UZ, dr, sS, RB, Dy, eM, nB, hk, agl, lF, cj, gZ, at, m, aPG, aMk); #c1(OCA2) c2(XP_011519942) c3(P protein isoform X2) c4(314739, 314740) c5( ) c6( ) c7(bL, sc, T, yk, iW, fr, bHA, q, rc, hX, cmw, av, hd, afQ, aT, SH, cmx, dL, afu, d, lR, aW, Y, ff, ih, apn, XC, eZ, lo, CQ, en, bHy, t, cm, UZ, dr, sS, RB, Dy, eM, nB, hk, agl, lF, cj, gZ, at, m, aPG, aMk); #c1(OCA2) c2(XP_011519943) c3(P protein isoform X3) c4( ) c5(314741, 314742, 314743, 314744, 314745, 314746) c6( ) c7(bL, sc, T, yk, iW, fr, bHA, q, rc, hX, cmw, av, hd, afQ, aT, SH, cmx, dL, afu, d, lR, aW, Y, ff, ih, apn, XC, eZ, lo, CQ, en, bHy, t, cm, UZ, dr, sS, RB, Dy, eM, nB, hk, agl, lF, cj, gZ, at, m, aPG, aMk); #c1(DCA2) c2(XP_011519945) c3(P protein isoform X5) c4( ) c5(314747, 314748, 314749, 314750, 314751, 314752) c6( ) c7(bL, sc, T, yk, iW, fr, bHA, q, rc, hX, cmw, av, hd, afQ, aT, SH, cmx, dL, afu, d, lR, aW, Y, ff, ih, apn, XC, eZ, lo, CQ, en, bHy, t, cm, UZ, dr, sS, RB, Dy, eM, nB, hk, agl, lF, cj, gZ, at, m, aPG, aMk); #c1(OCA2) c2(XP_011519948) c3(P protein isoform X8) c4(314753) c5(314754, 314755, 314756, 314757, 314758, 314759) c6( ) c7(bL, sc, T, yk, iW, fr, bHA, q, rc, hX, cmw, av, hd, afQ, aT, SH, cmx, dL, afu, d, lR, aW, Y, ff, ih, apn, XC, eZ, lo, CQ, en, bHy, t, cm, UZ, dr, sS, RB, Dy, eM, nB, hk, agl, lF, cj, gZ, at, m, aPG, aMk); #c1(OCA2) c2(XP_011519949) c3(P protein isoform X9) c4( ) c5(314760, 314761, 314762, 314763, 314764, 314765, 314766, 314767, 314768, 314769, 314770, 314771) c6( ) c7(bL, sc, T, yk, iW, fr, bHA, q, rc, hX, cmw, av, hd, afQ, aT, SH, cmx, dL, afu, d, lR, aW, Y, ff, ih, apn, XC, eZ, lo, CQ, en, bHy, t, cm, UZ, dr, sS, RB, Dy, eM, nB, hk, agl, lF, cj, gZ, at, m, aPG, aMk); #c1(OCIAD1) c2(NP_001073308) c3(OCIA domain-containing protein 1 isoform 1) c4(35775, 35776, 35777, 35778, 35779, 35780) c5(35781, 35782, 35783, 35784, 35785, 35786, 35787, 35788, 35789, 35790, 35791, 35792, 35793, 35794, 35795, 35796, 35797, 35798, 35799) c6( ) c7(t); #c1(OCIAD1) c2(NP_001073310) c3(OCIA domain-containing protein 1 isoform 3) c4(35800) c5(35801, 35802, 35803, 35804, 35805, 35806, 35807, 35808, 35809, 35810, 35811, 35812, 35813, 35814, 35815, 35816, 35817, 35818, 35819, 35820, 35821, 35822, 35823, 35824, 35825, 35826, 35827, 35828, 35829, 35830, 35831, 35832, 35833, 35834) c6( ) c7(t); #c1(OCIAD1) c2(NP_001073311) c3(OCIA domain-containing protein 1 isoform 2) c4(35835, 35836) c5(35837, 35838, 35839, 35840, 35841, 35842, 35843) c6( ) c7(t); #c1(OCIAD1) c2(NP_001161726) c3(OCIA domain-containing protein 1 isoform 4) c4( ) c5(65885, 65886, 65887, 65888, 65889, 65890, 65891, 65892, 65893, 65894) c6( ) c7(t); #c1(OCIAD2) c2(NP_001014446) c3(OCIA domain-containing protein 2 isoform 1) c4(25936) c5(25937, 25938, 25939, 25940, 25941, 25942) c6( ) c7(af, g, iE); #c1(OCIAD2) c2(NP_001273702) c3(OCIA domain-containing protein 2 isoform 2) c4(105664, 105665) c5(105666, 105667, 105668, 105669, 105670, 105671, 105672, 105673, 105674, 105675, 105676, 105677) c6( ) c7(af, g, iE); #c1(OCLM) c2(NP_07/770) c3(oculomedin) c4(212029) c5(212030, 212031, 212032, 212033, 212034, 212035) c6( ) c7(dw, dH); #c1(OCLN) c2(NP_001192183) c3(occludin isoform a) c4(82005, 82006, 82007) c5(82008, 82009, 82010, 82011, 82012, 82013, 82014, 82015, 82016, 82017, 82018, 82019, 82020, 82021, 82022, 82023) c6( ) c7(Dw, T, jx, aWU, ci, fu, hF, V, fU, aE, zf, aT, K, aPB, lR, aW, fe, aq, g, bz, ac, c, wz, E, aS, mk, adg, ra, B); #c1(OCLN) c2(NP_001192184) c3(occludin isoform b precursor) c4(82024) c5(82025, 82026, 82027, 82028, 82029, 82030, 82031, 82032, 82033, 82034, 82035, 82036) c6( ) c7(Dw, T, jx, aWU, ci, fu, hF, V, fU, aE, zf, aT, K, aPB, lR, aW, fe, aq, g, bz, ac, c, wz, E, aS, mk, adg, ra, B); #c1(DCM) c2(XP_011513787) c3(oncomodulin-1 isoform X1) c4(306245) c5(306246, 306247, 306248, 306249, 306250, 306251, 306252, 306253, 306254, 306255) c6( ) c7(Y, cbM, aq, auN, B, iP); #c1(OCM2) c2(NP_006179) c3(putative oncomodulin-2) c4(162622) c5(162623, 162624, 162625, 162626, 162627, 162628) c6( ) c7(Y, iP); #c1(OCRL) c2(NP_000267) c3(inositol polyphosphate 5-phosphatase OCRL-1 isoform a) c4(5940) c5(5941, 5942, 5943, 5944, 5945, 5946) c6( ) c7(aPt, azU, Tl, KW, dw, nf, nA, lM, aY, jr); #c1(OCRL) c2(NP_001578) c3(inositol polyphosphate 5-phosphatase OCRL-l isoform b) c4(118996) c5(118997, 118998, 118999, 119000, 119001, 119002) c6( ) c7(aPt, azU, Tl, KW, dw, nf, nA, iM, aY, jr); #c1(ODAM) c2(NP_060325) c3(odontogenic ameloblast-associated protein precursor) c4( ) c5(195403, 195404, 195405, 195406, 195407, 195408, 195409) c6(195410, 195411) c7(T, aW, Y, aT, ci, ahv); #c1(ODAM) c2(XP_005265755) c3(odontogenic ameloblast-associated protein isoform X1) c4(279706) c5(279707) c6( ) c7(T, aW, Y, aT, ci, ahv); #c1(ODC1) c2(NP_001274117) c3(ornithine decarboxylase isoform 2) c4( ) c5(106029, 106030, 106031, 106032, 106033) c6( ) c7(T, aC, iV, bbW, eR, aD, e, ha, ht, ff, aA, bB, n, gA, bS, j, ct, ci, fd, q, ad, eJ, ip, bv, dY, lg, aE, hb, dE, pX, aT, h, cy, dz, hn, K, jj, XZ, hu, ae, B, s, et, iU, aW, Y, aoo, az, baT, eH, r, bod, aMA, aq, aF, iy, kt, en, Sm, Z, g, eF, bz, cm, iR, ek, dR, ck, bF, ac, eM, bE, wO, hk, Pl, X, jh, gX, cj, qF, MC, y, kS, ne, ao); #c1(ODC1) c2(NP_001274119) c3(ornithine decarboxylase isoform 1) c4(106034, 106035, 106036) c5(106037, 106038, 106039, 106040, 106041, 106042, 106043, 106044, 106045, 106046, 106047, 106048, 106049, 106050, 106051, 106052, 106053) c6( ) c7(T, aC, iV, bbW, eR, aD, e, ha, ht, ff, aA, bB, n, gA, bS, j, ct, ci, fd, q, ad, eJ, ip, bv, dY, lg, aE, hb, dE, pX, aT, h, cy, dz, hn, K, jj, XZ, hu, ae, B, s, et, iU, aW, Y, aoo, az, baT, eH, r, bod, aMA, aq, aF, iy, kt, en, Sm, Z, g, eF, bz, cm, iR, ek, dR, ck, bF, ac, eM, bE, wO, hk, Pl, X, jh, gX, cj, qF, MC, y, kS, ne, ao); #c1(ODF1) c2(NP_077721) c3(outer dense fiber protein 1) c4(215293) c5(215294, 215295, 215296, 215297, 215298, 215299) c6( ) c7(zt, aj, pl, y, fq, J); #c1(ODF3B) c2(NP_001014440) c3(outer dense fiber protein 3B) c4(25910) c5(25911, 25912, 25913, 25914, 25915, 25916, 25917, 25918, 25919, 25920, 25921) c6( ) c7(V, kl); #c1(ODF4) c2(NP_694552) c3(outer dense fiber protein 4) c4(245277) c5(245278, 245279, 245280, 245281, 245282, 245283) c6( ) c7(sS); #c1(OFCC1) c2(XP_011513347) c3(orofacial cleft 1 candidate gene 1 protein) c4(305591) c5(305592, 305593, 305594, 305595, 305596, 305597) c6( ) c7(abZ, azs, he); #c1(OFD1) c2(NP_003602) c3(oral-facial-digital syndrome 1 protein) c4(139502, 139503) c5(139504, 139505, 139506, 139507, 139508, 139509, 139510, 139511, 139512, 139513, 139514, 139515) c6( ) c7(afR, FB, arV, dO, wB, cmz, anL, bsi, Nj, nA, ahD, cmy); #c1(OFD1) c2(XP_005274661) c3(oral-facial-digital syndrome 1 protein isoform X5) c4(285294, 285295) c5(285296, 285297, 285298, 285299, 285300, 285301, 285302, 285303, 285304, 285305, 285306, 285307) c6( ) c7(afR, FB, arV, dO, wB, cmz, anL, bsi, Nj, nA, ahD, cmy); #c1(OFD1) c2(XP_005274664) c3(oral-facial-digital syndrome 1 protein isoform X10) c4(285308) c5(285309, 285310, 285311, 285312, 285313) c6( ) c7(afR, FB, arV, dO, wB, cmz, anL, bsi, Nj, nA, ahD, cmy); #c1(OFD1) c2(XP_011543897) c3(oral-facial-digital syndrome 1 protein isoform X9) c4( ) c5(343076) c6( ) c7(afR, FB, arV, dO, wB, cmz, anL, bsi, Nj, nA, ahD, cmy); #c1(OGDH) c2(NP_001003941) c3(2-oxoglutarate dehydrogenase, mitochondrial isoform 2 precursor) c4(20310, 20311) c5(20312, 20313, 20314, 20315, 20316, 20317, 20318, 20319, 20320, 20321, 20322, 20323) c6( ) c7(ajU, U, cmA, j, K, af, aR, bc); #c1(OGDH) c2(NP_001158508) c3(2-oxoglutarate dehydrogenase, mitochondrial isoform 3 precursor) c4(63692) c5(63693, 63694, 63695, 63696, 63697, 63698) c6( ) c7(ajU, U, cmA, j, K, af, aR, bc); #c1(OGDH) c2(NP_002532) c3(2-oxoglutarate dehydrogenase, mitochondrial isoform 1 precursor) c4(129598) c5(129599, 129600, 129601, 129602, 129603, 129604) c6( ) c7(ajU, U, cmA, j, K, af, aR, bc); #c1(OGDH) c2(XP_005249818) c3(2-oxoglutarate dehydrogenase, mitochondrial isoform X2) c4(269888) c5(269889, 269890, 269891, 269892, 269893, 269894) c6( ) c7(ajU, U, cmA, j, K, af, aR, bc); #c1(OGDHL) c2(NP_001137468) c3(2-oxoglutarate dehydrogenase-like, mitochondrial isoform b) c4(55898, 55899) c5(55900, 55901, 55902, 55903, 55904, 55905) c6( ) c7(T, if, dy, ac, qN); #c1(OGDHL) c2(NP_060715) c3(2-oxoglutarate dehydrogenase-like, mitochondrial isoform a) c4(197417, 197418) c5(197419, 197420, 197421, 197422, 197423, 197424, 197425, 197426, 197427, 197428, 197429, 197430) c6( ) c7(T, if, dy, ac, qN); #c1(OGFOD1) c2(NP_060703) c3(prolyl 3-hydroxylase OGFOD1) c4(197300, 197301) c5(197302, 197303, 197304, 197305, 197306, 197307, 197308, 197309, 197310, 197311, 197312) c6( ) c7(cy, aw, j, cj); #c1(OGFOD1) c2(XP_011521500) c3(prolyl 3-hydroxylase OGFOD1 isoform X1) c4( ) c5(316246, 316247, 316248, 316249, 316250) c6( ) c7(cy, aw, j, cj); #c1(OGFR) c2(NP_031372) c3(opioid growth factor receptor) c4(171988, 171989, 171990, 171991, 171992, 171993) c5(171994, 171995, 171996, 171997, 171998, 171999, 172000, 172001, 172002, 172003, 172004, 172005, 172006, 172007, 172008, 172009, 172010, 172011, 172012, 172013, 172014, 172015, 172016, 172017, 172018, 172019, 172020, 172021, 172022, 172023, 172024, 172025, 172026, 172027, 172028, 172029, 172030) c6( ) c7(qJ, T, hd, aC, aE, nM, nB, m, t, ik, X); #c1(OGG1) c2(NP_002533) c3(N-glycosylase/DNA lyase isoform 1a) c4(129605) c5(129606, 129607, 129608, 129609, 129610, 129611) c6( ) c7(oO, T, aC, fR, atj, if, bB, ci, aY, ip, aE, u, Qk, K, hu, eL, Zm, dH, aW, CT, LZ, nY, kt, vs, g, bw, lk, qJ, xd, fT, eM, vA, Gs, U, Zn, k, m, Vj, iu, FW, gU, sw, aWG, ht, Pi, NT, n, aj, fr, ct, qN, jz, hw, h, eV, Gw, dL, hB, Xh, dx, ny, FZ, fQ, aq, op, Sc, W, dB, hk, Qu, hL, at, nL, cB, B, it, bL, eR, yk, eX, aA, cz, hy, v, j, V, q, J, eA, hb, aT, HG, bq, ae, af, ne, jf, iF, qw, Sh, s, Y, r, en, cl, t, kC, iK, ck, sS, rR, kP, pZ, alt, gX, JH, bt, rA, ao, Gl, hl, wi, iW, jh, bS, Gn, hF, ad, hX, dP, av, l, ib, py, sN, io, lQ, cmB, BN, BO, Ub, ff, aF, Z, bz, bF, es, bE, gZ, Gu, y, X, rk); #c1(OGG1) c2(NP_058212) c3 (N-glycosylase/DNA lyase isoform 1b) c4(192674) c5(192675, 192676, 192677, 192678, 192679) c6( ) c7(oO, T, aC, fR, atj, if, bB, ci, aY, ip, aE, u, Qk, K, hu, eL, Zm, dH, aW, CT, LZ, nY, kt, vs, g, bw, lk, qJ, xd, fT, eM, vA, Gs, U, Zn, k, m, Vj, iu, FW, gU, sw, aWG, ht, Pi, NT, n, aj, fr, ct, qN, jz, hw, h, eV, Gw, dL, hB, Xh, dx, ny, FZ, fQ, aq, op, Sc, W, dB, hk, Qu, hl, at, nL, cB, B, it, bL, eR, yk, ex, aA, cz, hy, v, j, V, q, J, eA, hb, aT, HG, bq, ae, af, ne, jf, iF, qw, Sh, s, Y, r, en, cl, t, kC, ik, ck, sS, rR, kP, pZ, alt, gX, JH, bt, rA, ao, Gl, hl, wi, iW, jh, bS, Gn, hF, ad, hX, dP, av, l, ib, py, sN, io, lQ, cmB, BN, BO, Ub, ff, aF, Z, bz, bF, es, bE, gZ, Gu, y, X, rk); #c1(OGG1) c2(NP_058213) c3(N-glycosylase/DNA lyase isoform 1c) c4(192680) c5(192681, 192682, 192683, 192684, 192685, 192686) c6( ) c7(oO, T, aC, fR, atj, if, bB, ci, aY, ip, aE, u, Qk, K, hu, eL, Zm, dH, aW, CT, LZ, nY, kt, vs, g, bw, lk, qJ, xd, fT, eM, vA, Gs, U, Zn, k, m, Vj, iu, FW, gU, sw, aWG, ht, Pi, NT, n, aj, fr, ct, qN, jz, hw, h, eV, Gw, dL, hB, Xh, dx, ny, FZ, fQ, aq, op, Sc, W, dB, hk, Qu, hl, at, nL, cB, B, it, bL, eR, yk, eX, aA, cz, hy, v, j, V, q, J, eA, hb, aT, HG, bq, ae, af, ne, jf, iF, qw, Sh, s, Y, r, en, cl, t, kC, iK, ck, sS, rR, kP, pZ, aJt, gX, JH, bt, rA, ao, Gl, hl, wi, iW, jh, bS, Gn, hF, ad, hX, dP, av, l, ib, py, sN, io, lQ, cmB, BN, BO, Ub, ff, aF, Z, bz, bF, es, bE, gZ, Gu, y, X, rk); #c1(OGG1) c2(NP_058214) c3(N-glycosylase/DNA lyase isoform 2a) c4(192687) c5(192688, 192689, 192690, 192691, 192692, 192693, 192694) c6( ) c7(oO, T, aC, fR, atj, if, bB, ci, aY, ip, aE, u, Qk, K, hu, eL, Zm, dH, aW, CT, LZ, nY, kt, vs, g, bw, lk, qJ, xd, fT, eM, vA, Gs, U, Zn, k, m, Vj, iu, FW, gU, sw, aWG, ht, Pi, NT, n, aj, fr, ct, qN, jz, hw, h, eV, Gw, dL, hB, Xh, dx, ny, FZ, fQ, aq, op, Sc, W, dB, hk, Qu, hl, at, nL, cB, B, it, bL, eR, yk, eX, aA, cz, hy, v, j, V, q, J, eA, hb, aT, HG, bq, ae, af, ne, jf, iF, qw, Sh, s, Y, r, en, cl, t, kC, iK, ck, sS, rR, kP, pZ, alt, gX, JH, bt, rA, ao, Gl, hl, wi, iW, jh, bS, Gn, hF, ad, hX, dP, av, l, ib, py, sN, io, lQ, cmB, BN, BO, Ub, ff, af, Z, bz, bF, es, bE, gZ, Gu, y, X, rk); #c1(OGG1) c2(NP_058434) c3(N-glycosylase/DNA lyase isoform 2b) c4(192709, 192710) c5(192711, 192712, 192713, 192714, 192715, 192716, 192717, 192718, 192719, 192720, 192721, 192722) c6( ) c7(oO, T, aC, fR, atj, if, bB, ci, aY, ip, aE, u, Qk, K, hu, eL, Zm, dH, aW, CT, LZ, nY, kt, vs, g, bw, lk, qJ, xd, fT, eM, vA, Gs, U, Zn, k, m, Vj, iu, FW, gU, sw, aWG, ht, Pi, NT, n, aj, fr, ct, qN, jz, hw, h, eV, Gw, dL, hB, Xh, dx, ny, FZ, fQ, aq, op, Sc, W, dB, hk, Qu, hL, at, nL, cB, B, it, bL, eR, yk, ex, aA, cz, hy, v, j, V, q, J, eA, hb, aT, HG, bq, ae, af, ne, jf, iF, qw, Sh, s, Y, r, en, cl, t, kC, ik, ck, sS, rR, kP, pZ, alt, gX, JH, bt, rA, ao, Gl, hl, wi, iW, jh, bS, Gn, hF, ad, hX, dP, av, l, ib, py, sN, io, lQ, cmB, BN, BO, Ub, ff, aF, Z, bz, bF, es, bE, gZ, Gu, y, X, rk); #c1(OGG1) c2(NP_058436) c3(N-glycosylase/DNA lyase isoform 2c) c4(192723) c5(192724, 192725, 192726, 192727, 192728, 192729) c6( ) c7(oO, T, aC, fR, atj, if, bB, ci, aY, ip, aE, u, Qk, K, hu, eL, Zm, dH, aW, CT, LZ, nY, kt, vs, g, bw, lk, qJ, xd, fT, eM, vA, Gs, U, Zn, k, m, Vj, iu, FW, gU, sw, aWG, ht, Pi, NT, n, aj, fr, ct, qN, jz, hw, h, eV, Gw, dL, hB, Xh, dx, ny, FZ, fQ, aq, op, Sc, W, dB, hk, Qu, hl, at, nL, cB, B, it, bL, eR, yk, eX, aA, cz, hy, v, j, V, q, J, eA, hb, aT, HG, bq, ae, af, ne, jf, iF, qw, Sh, s, Y, r, en, cl, t, kC, iK, ck, sS, rR, kP, pZ, alt, gX, JH, bt, rA, ao, Gl, hl, wi, iW, jh, bS, Gn, hF, ad, hX, dP, av, l, ib, py, sN, io, lQ, cmB, BN, BO, Ub, ff, aF, Z, bz, bF, es, bE, gZ, Gu, y, X, rk); #c1(OGG1) c2(NP_058437) c3(N-glycosylase/DNA lyase isoform 2d) c4(192730) c5(192731, 192732, 192733, 192734, 192735, 192736) c6( ) c7(oO, T, aC, fR, atj, if, bB, ci, aY, ip, aE, u, Qk, K, hu, eL, Zm, dH, aW, CT, LZ, nY, kt, vs, g, bw, lk, qJ, xd, fT, eM, vA, Gs, U, Zn, k, m, Vj, iu, FW, gU, sw, aWG, ht, Pi, NT, n, aj, fr, ct, qN, jz, hw, h, eV, Gw, dL, hB, Xh, dx, ny, FZ, fQ, aq, op, Sc, W, dB, hk, Qu, hl, at, nL, cB, B, it, bL, eR, yk, eX, aA, cz, hy, v, j, V, q, J, eA, hb, aT, HG, bq, ae, af, ne, jf, iF, qw, Sh, s, Y, r, en, cl, t, kC, iK, ck, sS, rR, kP, pZ, alt, gX, JH, bt, rA, ao, Gl, hl, wi, iW, jh, bS, Gn, hF, ad, hX, dP, av, l, ib, py, sN, io, lQ, cmB, BN, BO, Ub, ff, aF, Z, bz, bF, es, bE, gZ, Gu, y, X, rk); #c1(OGG1) c2(NP_058438) c3(N-glycosylase/DNA lyase isoform 2e) c4(192737) c5(192738, 192739, 192740, 192741, 192742, 192743) c6( ) c7(oO, T, aC, fR, atj, if, bB, ci, aY, ip, aE, u, Qk, K, hu, eL, Zm, dH, aW, CT, LZ, nY, kt, vs, g, bw, lk, qJ, xd, fT, eM, vA, Gs, U, Zn, k, m, Vj, iu, FW, gU, sw, aWG, ht, Pi, NT, n, aj, fr, ct, qN, jz, hw, h, eV, Gw, dL, hB, Xh, dx, ny, FZ, fQ, aq, op, Sc, W, dB, hk, Qu, hL, at, nL, cB, B, it, bL, eR, yk, eX, aA, cz, hy, v, j, V, q, J, eA, hb, aT, HG, bq, ae, af, ne, jf, iF, qw, Sh, s, Y, r, en, cl, t, kC, ik, ck, sS, rR, kP, pZ, alt, gX, JH, bt, rA, ao, Gl, hl, wi, iW, jh, bS, Gn, hF, ad, hX, dP, av, l, ib, py, sN, io, lQ, cmB, BN, BO, Ub, ff, aF, Z, bz, bF, es, bE, gZ, Gu, y, X, rk); #c1(OGG1) c2(XP_011532063) c3(N-glycosylase/DNA lyase isoform X2) c4( ) c5(328132) c6( ) c7(oO, T, aC, fR, atj, if, bB, ci, aY, ip, aE, u, Qk, K, hu, eL, Zm, dH, aW, CT, LZ, nY, kt, vs, g, bw, lk, qJ, xd, fT, eM, vA, Gs, U, Zn, k, m, Vj, iu, FW, gU, sw, aWG, ht, Pi, NT, n, aj, fr, ct, qN, jz, hw, h, eV, Gw, dL, hB, Xh, dx, ny, FZ, fQ, aq, op, Sc, W, dB, hk, Qu, hL, at, nL, cB, B, it, bL, eR, yk, ex, aA, cz, hy, v, j, V, q, J, eA, hb, aT, HG, bq, ae, af, ne, jf, iF, qw, Sh, s, Y, r, en, cl, t, kC, iK, ck, sS, rR, kP, pZ, alt, gX, JH, bt, rA, ao, Gl, hl, wi, iW, jh, bS, Gn, hF, ad, hX, dP, av, l, ib, py, sN, io, lQ, cmB, BN, BO, Ub, ff, aF, Z, bz, bF, es, bE, gZ, Gu, y, X, rk); #c1(OGN) c2(NP_054776) c3(mimecan preproprotein) c4(177732, 177733) c5(177734, 177735, 177736, 177737, 177738, 177739, 177740, 177741, 177742, 177743, 177744, 177745, 177746, 177747, 177748, 177749) c6( ) c7(hR, s); #c1(OGT) c2(NP_858058) c3(UDP-N-acetylglucosamine-peptide N-acetylglucosaminyltransferase 110 kDa subunit isoform 1) c4(255326) c5(255327, 255328, 255329, 255330, 255331, 255332) c6( ) c7(bL, T, Hx, aC, aE, AO, BA, aW, aT, aU, n, aj, K, C, bq, t, ae, cd, J, ad, vQ); #c1(OGT) c2(NP_858059) c3(UDP-N-acetylglucosamine-peptide N-acetylglucosaminyltransferase 110 kDa subunit isoform 2) c4(255333) c5(255334, 255335, 255336, 255337, 255338, 255339) c6( ) c7(bL, T, Hx, aC, aE, AO, BA, aW, aT, aU, n, aj, K, C, bq, t, ae, cd, J, ad, vQ); #c1(DIP5) c2(NP_009211) c3(protein Mis18-beta) c4(171539) c5(171540, 171541, 171542, 171543, 171544, 171545) c6( ) c7(T, ff, ht, aA, v, ey, af, en, hu); #c1(DIT3) c2(NP_689848) c3(oncoprotein-induced transcript 3 protein precursor) c4(244142, 244143) c5(244144, 244145, 244146, 244147, 244148, 244149, 244150, 244151, 244152, 244153, 244154, 244155) c6( ) c7(nH, B); #c1(OLA1) c2(NP_001011708) c3(obg-like ATPase 1 isoform 2) c4( ) c5(24765, 24766, 24767, 24768, 24769, 24770, 24771, 24772, 24773, 24774) c6( ) c7(aW, aT); #c1(OLA1) c2(NP_037473) c3(obg-like ATPase 1 isoform 1) c4(175692, 175693, 175694, 175695, 175696, 175697) c5(175698, 175699, 175700, 175701, 175702, 175703, 175704, 175705, 175706, 175707, 175708, 175709, 175710, 175711, 175712, 175713, 175714, 175715, 175716, 175717, 175718, 175719, 175720, 175721, 175722, 175723) c6( ) c7(aW, aT); #c1(OLFM1) c2(NP_001269540) c3(noelin isoform 4 precursor) c4(101842, 101843) c5(101844, 101845, 101846, 101847, 101848, 101849, 101850, 101851, 101852, 101853, 101854, 101855, 101856, 101857, 101858, 101859) c6( ) c7(F, af, aq, va, f, H, iJ, B, ad, L); #c1(OLFM1) c2(NP_001269541) c3(noelin isoform 5) c4(101860) c5(101861, 101862, 101863, 101864, 101865, 101866, 101867, 101868) c6( ) c7(F, af, aq, va, f, H, iJ, B, ad, L); #c1(OLFM1) c2(NP_006325) c3(noelin isoform 2 precursor) c4( ) c5(164040, 164041, 164042, 164043, 164044, 164045, 164046, 164047) c6( ) c7(F, af, aq, va, f, H, iJ, B, ad, L); #c1(OLFM1) c2(NP_055094) c3(noelin isoform 1 precursor) c4(178888, 178889) c5(178890, 178891, 178892, 178893, 178894, 178895, 178896, 178897) c6( ) c7(F, af, aq, va, f, H, iJ, B, ad, L); #c1(OLFM2) c2(NP_477512) c3(noelin-2 isoform 2 precursor) c4(230782) c5(230783, 230784, 230785, 230786, 230787, 230788, 230789, 230790) c6(230791, 230792) c7(pZ); #c1(OLFM4) c2(NP_006409) c3(olfactomedin-4 precursor) c4(164781) c5(164782, 164783, 164784, 164785, 164786, 164787) c6( ) c7(T, aA, bB, v, j, ci, q, ad, dP, aE, ae, ev, r, aF, cl, fq, ac, c, iT, bE, gX, hL, gZ, bt); #c1(OLFML2B) c2(NP_056256) c3 (olfactomedin-like protein 2B isoform 2 precursor) c4(186772) c5(186773, 186774, 186775, 186776, 186777, 186778, 186779, 186780) c6(186781, 186782) c7(dD, eq, EQ); #c1(OLFML2B) c2(XP_005245132) c3(olfactomedin-like protein 2B isoform X1) c4(266315) c5( ) c6( ) c7(dD, eq, EQ); #c1(OLFML2B) c2(XP_011507700) c3(olfactomedin-like protein 28 isoform X2) c4( ) c5(297699, 297700, 297701, 297702, 297703, 297704, 297705, 297706) c6(297707, 297708) c7(dD, eq, EQ); #c1(OLIG1) c2(NP_620450) c3(oligodendrocyte transcription factor 1) c4(237658, 237659, 237660) c5(237661, 237662, 237663, 237664, 237665, 237666, 237667, 237668, 237669, 237670, 237671, 237672, 237673, 237674, 237675, 237676, 237677, 237678, 237679) c6( ) c7(byp, av, Fc, d, eM, dB, yL, qy, rE, as, cb, sj, V, eA, f, qc); #c1(OLlG2) c2(XP_005260965) c3(oligodendrocyte transcription factor 2 isoform X1) c4(277030, 277031) c5(277032, 277033, 277034, 277035, 277036, 277037, 277038, 277039, 277040, 277041, 277042, 277043, 277044, 277045, 277046, 277047, 277048, 277049) c6( ) c7(T, Tb, cfg, qy, ci, bNe, Fc, eA, iC, iX, aT, By, Ou, K, cb, he, rE, af, dx, jf, iF, Uf, d, aW, Y, yL, apk, as, f, Hp, ck, chr, c, dB, bt, qc); #c1(OLlG3) c2(NP_786923) c3(oligodendrocyte transcription factor 3) c4(251909) c5(251910, 251911, 251912, 251913, 251914, 251915) c6( ) c7(bL, A, oe, cr, cz, gZ, j, hp, iV, y, V, fq); #c1(OLR1) c2(NP_001166103) c3(oxidized low-density lipoprotein receptor 1 isoform 2) c4(68159) c5(68160, 68161, 68162, 68163, 68164, 68165) c6( ) c7(bL, T, dD, eR, ea, e, aw, l, cz, cR, aj, v, j, eq, cd, J, cc, adL, Bc, Ot, M, aT, ey, K, dQ, eN, af, hm, cH, cW, aW, hl, uC, qa, kT, ac, mf, aS, ei, y, L); #c1(OLR1) c2(NP_001166104) c3(oxidized low-density lipoprotein receptor 1 isoform 3) c4(68166) c5(68167, 68168, 68169, 68170, 68171, 68172) c6( ) c7(bL, T, dD, eR, ea, e, aw, l, cz, cR, aj, v, j, eq, cd, J, cc, adL, Bc, Ot, M, aT, By, K, dQ, eN, af, hm, cH, cW, aW, hl, uC, qa, KT, ac, mf, aS, ei, y, L); #c1(OLR1) c2(XP_011518984) c3(oxidized low-density lipoprotein receptor 1 isoform X1) c4(313458) c5(313459, 313460, 313461, 313462, 313463, 313464) c6( ) c7(bL, T, dD, eR, ea, e, aw, l, cz, cR, aj, v, j, eq, cd, J, cc, adL, Bc, Ot, M, aT, ey, K, dQ, eN, af, hm, cH, cW, aW, hl, uC, qa, kT, ac, mf, aS, ei, y, L); #c1(OMD) c2(NP_005005) c3(osteomodulin precursor) c4(152001) c5(152002, 152003, 152004, 152005, 152006, 152007, 152008, 152009, 152010, 152011) c6(152012, 152013) c7(rE, T, qc); #c1(OMG) c2(NP_002535) c3(oligodendrocyte-myelin glycoprotein precursor) c4(129612) c5(129613, 129614, 129615, 129616, 129617, 129618, 129619, 129620) c6( ) c7(ahN, Ew, ac, fm, aiz, hP, nA, V, bN); #c1(OMP) c2(NP_006180) c3(olfactory marker protein) c4(162629) c5(162630, 162631, 162632, 162633, 162634, 162635, 162636) c6( ) c7(at, alx, VF, oc); #c1(ONECUT1) c2(NP_004489) c3(hepatocyte nuclear factor 6) c4(146861) c5(146862, 146863, 146864, 146865, 146866, 146867, 146868) c6( ) c7(bL, T, aC, aE, bE, qt, ff, FN, aq, aj, kp, bz, en, fq, B); #c1(ONECUT2) c2(NP_004843) c3(one cut domain family member 2) c4(150209, 150210) c5(150211, 150212, 150213, 150214, 150215, 150216, 150217, 150218, 150219, 150220, 150221, 150222, 150223, 150224) c6( ) c7(dB, T, fM, EQ); #c1(OPA1) c2(NP_056375) c3(dynamin-like (20 kDa protein, mitochondrial isoform 1) c4(187342) c5(187343, 187344, 187345, 187346, 187347, 187348) c6( ) c7(cmC, HQ, cZ, T, cz, bD, aNG, cmD, ci, bgR, q, aR, cd, alD, cmE, aV, oj, vd, K, bU, dd, awL, nP, cdr, dH, mA, ff, wf, cdq, cds, en, g, dw, px, kC, vU, ac, c, pZ, mG, U, awk, gv, acJ, eQ, iM, B, Al); #c1(OPA1) c2(NP_570844) c3(dynamin-like (20 kDa protein, mitochondrial isoform 2) c4( ) c5(234296, 234297, 234298, 234299, 234300, 234301) c6( ) c7(cmC, HQ, cZ, T, cz, bD, aNG, cmD, ci, bgR, q, aR, cd, alD, cmE, aV, oj, vd, K, bU, dd, awL, nP, cdr, dH, mA, ff, wf, cdq, cds, en, g, dw, px, kC, vU, ac, c, pZ, mG, U, awK, gv, acJ, eL, iM, B, Al); #c1(OPA1) c2(NP_570846) c3(dynamin-like (20 kDa protein, mitochondrial isoform 4) c4(234302, 234303) c5(234304, 234305, 234306, 234307, 234308, 234309) c6( ) c7(cmC, HQ, cZ, T, cz, bD, aNG, cmD, ci, bgR, q, aR, cd, alD, cmE, aV, oj, vd, K, bU, dd, awL, nP, cdr, dH, mA, ff, wf, cdq, cds, en, g, dw, px, kC, vU, ac, c, pZ, mG, U, awK, gv, acJ, eQ, lM, B, Al); #c1(OPA1) c2(NP_570847) c3(dynamin-like (20 kDa protein, mitochondrial isoform 5) c4(234310, 234311) c5(234312, 234313, 234314, 234315, 234316, 234317, 234318, 234319, 234320, 234321, 234322, 234323) c6( ) c7(cmC, HQ, cZ, T, cz, bD, aNG, cmD, ci, bgR, q, aR, cd, alD, cmE, aV, oj, vd, K, bU, dd, awL, nP, cdr, dH, mA, ff, wf, cdq, cds, en, g, dw, px, kC, vU, ac, c, pZ, mG, U, awK, gv, acJ, eQ, lM, B, Al); #c1(OPA1) c2(NP_570848) c3(dynamin-like (20 kDa protein, mitochondrial isoform 6) c4(234324) c5(234325, 234326, 234327, 234328, 234329, 234330) c6( ) c7(cmC, HQ, cZ, T, cz, bD, aNG, cmD, ci, bgR, q, aR, cd, alD, cmE, aV, oj, vd, K, bU, dd, awL, nP, cdr, dH, mA, ff, wF, cdq, cds, en, g, dw, px, kC, vU, ac, c, pZ, mG, U, awK, gv, acJ, eQ, lM, B, Al); #c1(OPA1) c2(NP_570849) c3(dynamin-like 120 kDa protein, mitochondrial isoform 7) c4(234331) c5(234332, 234333, 234334, 234335, 234336, 234337) c6( ) c7(cmC, HQ, cZ, T, cz, bD, aNG, cmD, ci, bgR, q, aR, cd, alD, cmE, aV, oj, vd, K, bU, dd, awL, nP, cdr, dH, mA, ff, wF, cdq, cds, en, g, dw, px, kC, vU, ac, c, pZ, mG, U, awK, gv, acJ, eL, lM, B, Al); #c1(OPA1) c2(NP_570850) c3(dynamin-like 120 kDa protein, mitochondrial isoform 8) c4(234338) c5(234339, 234340, 234341, 234342, 234343, 234344) c6( ) c7(cmC, HQ, cZ, T, cz, bD, aNG, cmD, ci, bgR, q, aR, cd, alD, cmE, aV, oj, vd, K, bU, dd, awL, nP, cdr, dH, mA, ff, wF, cdq, cds, en, g, dw, px, kC, vU, ac, c, pZ, mG, U, awK, gv, acJ, eQ, lM, B, Al); #c1(OPA3) c2(NP_001017989) c3(optic atrophy 3 protein isoform a) c4(26732) c5(26733, 26734, 26735, 26736, 26737, 26738) c6( ) c7(alD, lR, cmH, cmG, awK, cmF, vd, eQ, lJ); #c1(OPA3) c2(NP_079412) c3(optic atrophy 3 protein isoform b) c4(218012, 218013) c5(218014, 218015, 218016, 218017, 218018, 218019, 218020) c6( ) c7(alD, lR, cmH, cmG, awk, cmF, vd, eQ, lJ); #c1(DPA3) c2(XP_005259335) c3(optic atrophy 3 protein isoform X2) c4( ) c5(276062, 276063, 276064, 276065, 276066) c6( ) c7(alD, lR, cmH, cmG, awk, cmF, vd, eQ, lJ); #c1(OPCML) c2(NP_001012393) c3(opioid-binding protein/cell adhesion molecule isoform b preproprotein) c4(24911, 24912) c5(24913, 24914, 24915, 24916, 24917, 24918, 24919, 24920, 24921, 24922, 24923, 24924) c6( ) c7(T, oe, e, eX, jh, aA, aj, ci, eA, XX, jz, eV, dL, K, eL, jf, B, PL, g, t, fq, dB, gv, m, iu); #c1(OPCML) c2(NP_002536) c3(opioid-binding protein/cell adhesion molecule isoform a preproprotein) c4(129621, 129622) c5(129623, 129624, 129625, 129626, 129627, 129628, 129629, 129630, 129631, 129632, 129633, 129634) c6( ) c7(T, oe, e, eX, jh, aA, aj, ci, eA, XX, jz, eV, dL, K, eL, jf, B, PL, g, t, fq, dB, gv, m, iu); #c1(OPHN1) c2(XP_005262327) c3 (oligophrenin-1 isoform X3) c4(277834) c5(277835, 277836, 277837, 277838, 277839, 277840, 277841, 277842, 277843, 277844, 277845) c6( ) c7(nd, nf, bS, cml, ct, nA, Z); #c1(DPHN1) c2(XP_011529263) c3(oligophrenin-1 isoform X1) c4(324707, 324708) c5(324709, 324710, 324711, 324712, 324713, 324714, 324715) c6( ) c7(nd, nf, bS, cml, ct, nA, Z); #c1(OPNILW) c2(NP_064445) c3(long-wave-sensitive opsin 1) c4(202490, 202491) c5(202492, 202493, 202494, 202495, 202496, 202497, 202498, 202499, 202500, 202501, 202502, 202503) c6( ) c7(T, cmJ, jh, it, bB, oY, fd, Kh, ci, C, nA, hF, AO, q, ad, aCi, eA, av, aT, h, ask, ae, pi, uQ, cmk, avP, je, lR, kS, aW, t, bz, all, ck, Qi, cmL, nM, iM, eM, kB, bE, cj, CZ, m, bt, xg, L); #c1(OPNIMW2) c2(NP_001041646) c3(medium-wave-sensitive opsin 1) c4(34236) c5(34237, 34238, 34239, 34240, 34241, 34242, 34243, 34244, 34245, 34246) c6( ) c7(jf, Hx, T, adB, Fw, cd); #c1(OPNISW) c2(NP_001699) c3(short-wave-sensitive opsin 1) c4(120120) c5(120121, 120122, 120123, 120124, 120125, 120126) c6( ) c7(T, fM, dA, al, jh, n, jE, qN, hX, x, iu, fX, pp, hn, cmM, nJ, iL, d, lR, iU, mA, hl, bnl, yq, eF, aGj, f, bz, Uk, hq, ck, bF, ou, gk, W, dc, k, hE, bt, B, jr); #c1(OPN3) c2(NP_055137) c3(opsin-3) c4(179220, 179221) c5(179222, 179223, 179224, 179225, 179226, 179227, 179228, 179229, 179230, 179231, (79232, 179233) c6( ) c7(bM, ft, dY, z, V, B); #c1(DPN4) c2(NP_001025186) c3(melanopsin isoform 2) c4(29471, 29472) c5(29473, 29474, 29475, 29476, 29477, 29478, 29479, 29480, 29481, 29482, 29483, 29484) c6( ) c7(T, os, aW, cr, aT, Nt, ta, oZ, j, ei, cm, ae, cd, ad, hZ); #c1(OPN4) c2(NP_150598) c3(melanopsin isoform 1) c4(228136) c5(228137, 228138, 228139, 228140, 228141, 228142) c6( ) c7(T, os, aW, cr, aT, Nt, ta, oZ, j, ei, cm, ae, cd, ad, hZ); #c1(OPN5) c2(NP_859528) c3(opsin-5) c4(255536) c5(255537, 255538, 255539, 255540, 255541, 255542) c6( ) c7(aW, he, B, bK, aT); #c1(OPRK1) c2(NP_000903) c3(kappa-type opioid receptor isoform 1) c4( ) c5(17065, 17066, 17067, 17068, 17069, 17070, 17071, 17072, 17073, 17074, 17075, (7076, 17077) c6( ) c7(jd, bO, jh, bS, gf, ct, VV, eA, br, x, FY, aqt, gM, Jb, rJ, pX, aqk, h, gl, K, ox, he, hZ, iU, cmN, cu, gu, Z, bw, FT, ck, ok, cA, eM, Gs, Ei, bt, cj, Rm, k, lB); #c1(OPRL1) c2(NP_001186948) c3(nociceptin receptor) c4( ) c5(78863, 78864, 78865, 78866, 78867, 78868, 78869, 78870, 78871, 78872, 78873) c6( ) c7(eY, T, VR, aCt, Ei, gu, cm, lB, FT, hZ); #c1(OPRL1) c2(XP_011527130) c3(nociceptin receptor isoform X1) c4(321634) c5(321635, 321636, 321637) c6( ) c7(eY, T, VR, aCt, Ei, gu, cm, lB, FT, hZ); #c1(OPRM1) c2(NP_000905) c3(mu-type opioid receptor isoform MOR-1) c4( ) c5(17078, 17079, 17080, 17081, 17082) c6( ) c7(oO, T, os, bO, gf, ur, VV, Gb, K, xa, bc, gr, iE, aW, hl, hJ, cm, eM, Gs, cj, sv, abl, aCS, gL, ct, cd, ai, FY, h, lG, aNU, dL, gl, hg, aTX, he, PT, fe, aFx, cM, Jh, hq, ok, at, aqm, lB, jd, cmP, bL, jc, jx, bAp, v, j, eA, Si, aT, cmO, bq, avl, ae, eu, cmN, Y, aPf, cu, en, xw, FT, bN, iQ, qX, VR, cmQ, Dd, ha, jh, Gd, oZ, bS, uu, ad, dP, F, aqk, Jc, hZ, Gv, vj, aCt, ff, gC, cr, gl, gu, aTR, Z, jb, bz, Ek, es, Cq, eU, xZ, HN, Rm, be, pk, hE); #c1(OPRM1) c2(NP_001008503) c3(mu-type opioid receptor isoform MOR-10) c4(23273) c5(23274, 23275, 23276, 23277, 23278, 23279, 23280) c6( ) c7(oO, T, os, bO, gf, ur, W, Gb, K, xa, bc, gr, iE, aW, hl, hJ, cm, eM, Gs, cj, sv, abl, aCS, gL, ct, cd, ai, FY, h, lG, aNU, dL, gl, hg, aTX, he, PT, fe, aFx, cM, Jh, hq, ok, at, aqm, lB, jd, cmP, bL, jc, jx, bAp, v, j, eA, Si, aT, cmO, bq, avl, ae, eu, cmN, Y, aPf, cu, en, xw, FT, bN, iQ, qX, VR, cmQ, Dd, ha, jh, Gd, oZ, bS, uu, ad, dP, F, aqk, Jc, hZ, Gv, vj, aCt, ff, gC, cr, gl, gu, aTR, Z, jb, bz, Ek, es, Cq, eU, xZ, HN, Rm, be, pk, hE); #c1(OPRM1) c2(NP_001008504) c3(mu-type opioid receptor isoform MOR-1A) c4(23281) c5(23282, 23283, 23284, 23285, 23286, 23287, 23288, 23289, 23290, 23291, 23292, 23293, 23294, 23295, 23296, 23297) c6( ) c7(oO, T, os, bO, gf, ur, WV, Gb, K, xa, bc, gr, iE, aW, hl, hJ, cm, eM, Gs, cj, sv, abl, aCS, gL, ct, cd, ai, FY, h, lG, aNU, dL, gl, hg, aTX, he, PT, fe, aFx, cM, Jh, hq, ok, at, aqm, lB, jd, cmP, bL, jc, jx, bAp, v, j, eA, Si, aT, cmO, bq, avl, ae, eu, cmN, Y, aPf, cu, en, xw, FT, bN, il, qX, VR, cmQ, Dd, ha, jh, Gd, oZ, bS, uu, ad, dP, F, aqk, Jc, hZ, Gv, vj, aCt, ff, gC, cr, gl, gu, aTR, Z, jb, bz, Ek, es, Cq, ell, xZ, HN, Rm, be, pK, hE); #c1(OPRM1) c2(NP_001008505) c3(mu-type opioid receptor isoform MOR-1X) c4(23298) c5(23299, 23300, 23301, 23302, 23303, 23304) c6( ) c7(oO, T, os, bO, gf, ur, VV, Gb, K, xa, bc, gr, iE, aW, hl, hJ, cm, eM, Gs, cj, sv, abl, aCS, gL, ct, cd, ai, FY, h, lG, aNU, dL, gl, hg, aTX, he, PT, fe, aFx, cM, Jh, hq, ok, at, aqm, lB, jd, cmP, bL, jc, jx, bAp, v, j, eA, Si, aT, cmO, bq, avl, ae, eu, cmN, Y, aPf, cu, en, xw, FT, bN, iQ, qX, VR, cmQ, Dd, ha, jh, Gd, oZ, bS, uu, ad, dP, F, aqk, Jc, hZ, Gv, vj, aCt, ff, gC, cr, gl, gu, aTR, Z, jb, bz, Ek, es, Cq, eU, xZ, HN, Rm, be, pk, hE); #c1(OPRM1) c2(NP_001138752) c3(mu-type opioid receptor isoform MOR-1G1) c4( ) c5(57527, 57528, 57529, 57530, 57531, 57532, 57533, 57534, 57535, 57536, 57537) c6( ) c7(oO, T, os, bO, gf, ur, WV, Gb, K, xa, bc, gr, iE, aW, hl, hJ, cm, eM, Gs, cj, sv, abl, aCS, gL, ct, cd, ai, FY, h, lG, aNU, dL, gl, hg, aTX, he, PT, fe, aFx, cM, Jh, hq, ok, at, aqm, lB, jd, cmP, bL, jc, jx, bAp, v, j, eA, Si, aT, cmO, bq, avl, ae, eu, cmN, Y, aPf, cu, en, xw, FT, bN, iQ, qX, VR, cmQ, Dd, ha, jh, Gd, oZ, bS, uu, ad, dP, F, aqk, Jc, hZ, Gv, vj, aCt, ff, gC, cr, gl, gu, aTR, Z, jb, bz, Ek, es, Cq, eU, xZ, HN, Rm, be, pk, hE); #c1(OPRM1) c2(NP_001138753) c3(mu-type opioid receptor isoform MOR-1G2) c4(57538, 57539) c5(57540, 57541, 57542, 57543, 57544, 57545, 57546, 57547, 57548) c6( ) c7(oO, T, os, bO, gf, ur, WV, Gb, K, xa, bc, gr, iE, aW, hl, hJ, cm, eM, Gs, cj, sv, abl, aCS, gL, ct, cd, ai, FY, h, lG, aNU, dL, gl, hg, aTX, he, PT, fe, aFx, cM, Jh, hq, ok, at, aqm, lB, jd, cmP, bL, jc, jx, bAp, v, j, eA, Si, aT, cmO, bq, avl, ae, eu, cmN, Y, aPf, cu, en, xw, FT, bN, iQ, qX, VR, cmQ, Dd, ha, jh, Gd, oZ, bS, uu, ad, dP, F, aqk, Jc, hZ, Gv, vj, aCt, ff, gC, cr, gl, gu, aTR, Z, jb, bz, Ek, es, Cq, eU, xZ, HN, Rm, be, pk, hE); #c1(OPRM1) c2(NP_001138754) c3(mu-type opioid receptor isoform MOR-1B1) c4(57549) c5(57550, 57551, 57552, 57553, 57554) c6( ) c7(oO, T, os, bO, gf, ur, VV, Gb, K, xa, bc, gr, iE, aW, hl, hJ, cm, eM, Gs, cj, sv, abl, aCS, gL, ct, cd, ai, FY, h, lG, aNU, dL, gl, hg, aTX, he, PT, fe, aFx, cM, Jh, hq, ok, at, aqm, lB, jd, cmP, bL, jc, jx, bAp, v, j, eA, Si, aT, cmO, bq, avl, ae, eu, cmN, Y, aPf, cu, en, xw, FT, bN, iQ, qX, VR, cmQ, Dd, ha, jh, Gd, oZ, bS, uu, ad, dP, F, aqk, Jc, hZ, Gv, vj, aCt, ff, gC, cr, gl, gu, aTR, Z, jb, bz, Ek, es, Cq, eU, xZ, HN, Rm, be, pk, hE); #c1(OPRM1) c2(NP_001138755) c3(mu-type opioid receptor isoform MOR-182) c4(57555) c5(57556, 57557, 57558, 57559, 57560) c6( ) c7(oO, T, os, bO, gf, ur, VV, Gb, K, xa, bc, gr, iE, aW, hl, hJ, cm, eM, Gs, cj, sv, abl, aCS, gL, ct, cd, ai, FY, h, lG, aNU, dL, gl, hg, aTX, he, PT, fe, aFx, cM, Jh, hq, ok, at, aqm, lB, jd, cmP, bL, jc, jx, bAp, v, j, eA, Si, aT, cmO, bq, avl, ae, eu, cmN, Y, aPf, cu, en, xw, FT, bN, iQ, qX, VR, cmQ, Dd, ha, jh, Gd, oZ, bS, uu, ad, dP, F, aqk, Jc, hZ, Gv, vj, aCt, ff, gC, cr, gl, gu, aTR, Z, jb, bz, Ek, es, Cq, eU, xZ, HN, Rm, be, pk, hE); #c1(OPRM1) c2(NP_001138756) c3(mu-type opioid receptor isoform MOR-183) c4(57561) c5(57562, 57563, 57564, 57565, 57566, 57567) c6( ) c7(oO, T, os, bO, gf, ur, VV, Gb, K, xa, bc, gr, iE, aW, hl, hJ, cm, eM, Gs, cj, sv, abl, aCS, gL, ct, cd, ai, FY, h, lG, aNU, dL, gl, hg, aTX, he, PT, fe, aFx, cM, Jh, hq, ok, at, aqm, lB, jd, cmP, bL, jc, jx, bAp, v, j, eA, Si, aT, cmO, bq, avl, ae, eu, cmN, Y, aPf, cu, en, xw, FT, bN, iQ, qX, VR, cmQ, Dd, ha, jh, Gd, oZ, bS, uu, ad, dP, F, aqk, Jc, hZ, Gv, vj, aCt, ff, gC, cr, gl, gu, aTR, Z, jb, bz, Ek, es, Cq, eU, xZ, HN, Rm, be, pk, hE); #c1(OPRM1) c2(NP_001138757) c3(mu-type opioid receptor isoform MOR-184) c4( ) c5(57568, 57569, 57570, 57571, 57572) c6( ) c7(oO, T, os, bO, gf, ur, VV, Gb, K, xa, bc, gr, iE, aW, hl, hJ, cm, eM, Gs, cj, sv, abl, aCS, gL, ct, cd, ai, FY, h, lG, aNU, dL, gl, hg, aTX, he, PT, fe, aFx, cM, Jh, hq, ok, at, aqm, lB, jd, cmP, bL, jc, jx, bAp, v, j, eA, Si, aT, cmO, bq, avl, ae, eu, cmN, Y, aPf, cu, en, xw, FT, bN, iQ, qX, VR, cmQ, Dd, ha, jh, Gd, oZ, bS, uu, ad, dP, F, aqk, Jc, hZ, Gv, vj, aCt, ff, gC, cr, gl, gu, aTR, Z, jb, bz, Ek, es, Cq, ell, xZ, HN, Rm, be, pk, hE); #c1(OPRM1) c2(NP_001138758) c3(mu-type opioid receptor isoform MOR-185) c4(57573) c5(57574, 57575, 57576, 57577, 57578, 57579) c6( ) c7(oO, T, os, bO, gf, ur, VV, Gb, K, xa, bc, gr, iE, aW, hl, hJ, cm, eM, Gs, cj, sv, abl, aCS, gL, ct, cd, ai, FY, h, lG, aNU, dL, gl, hg, aTX, he, PT, fe, aFx, cM, Jh, hq, ok, at, aqm, lB, jd, cmP, bL, jc, jx, bAp, v, j, eA, Si, aT, cmO, bq, avl, ae, eu, cmN, Y, aPf, cu, en, xw, FT, bN, iQ, qX, VR, cmQ, Dd, ha, jh, Gd, oZ, bS, uu, ad, dP, F, aqk, Jc, hZ, Gv, vj, aCt, ff, gC, cr, gl, gu, aTR, Z, jb, bz, Ek, es, Cq, eU, xZ, HN, Rm, be, pk, hE); #c1(OPRM1) c2(NP_001272451) c3(mu-type opioid receptor isoform MOR-1S) c4( ) c5(104062, 104063, 104064, 104065, 104066) c6( ) c7(oO, T, os, bO, gf, ur, W, Gb, K, xa, bc, gr, iE, aW, hl, hJ, cm, eM, Gs, cj, sv, abl, aCS, gL, ct, cd, ai, FY, h, lG, aNU, dL, gl, hg, aTX, he, PT, fe, aFx, cM, Jh, hq, ok, at, aqm, lB, jd, cmP, bL, jc, jx, bAp, v, j, eA, Si, aT, cmO, bq, avl, ae, eu, cmN, Y, aPf, cu, en, xw, FT, bN, iQ, qX, VR, cmQ, Dd, ha, jh, Gd, oZ, bS, uu, ad, dP, F, aqk, Jc, hZ, Gv, vj, aCt, ff, gC, cr, gl, gu, aTR, Z, jb, bz, Ek, es, Cq, eU xZ, HN, Rm, be, pk, hE); #c1(OPRM1) c2(NP_001272453) c3(mu-type opioid receptor isoform MOR-1i) c4(104067, 104068, 104069, 104070, 104071, 104072) c5(104073, 104074, 104075, 104076, 104077, 104078, 104079, 104080, 104081, 104082, 104083, 104084, 104085, 104086, 104087, 104088, 104089, 104090, 104091, 104092) c6( ) c7(oO, T, os, bO, gf, ur, VV, Gb, K, xa, bc, gr, iE, aW, hl, hJ, cm, eM, Gs, cj, sv, abl, aCS, gL, ct, cd, ai, FY, h, lG, aNU, dL, gl, hg, aTX, he, PT, fe, aFx, cM, Jh, hq, ok, at, aqm, lB, jd, cmP, bL, jc, jx, bAp, v, j, eA, Si, aT, cmO, bq, avl, ae, eu, cmN, Y, aPf, cu, en, xw, FT, bN, iQ, qX, VR, cmQ, Dd, ha, jh, Gd, oZ, bS, uu, ad, dP, F, aqk, Jc, hZ, Gv, vj, aCt, ff, gC, cr, gl, gu, aTR, Z, jb, bz, Ek, es, Cq, eU, xZ, HN, Rm, be, pk, hE); #c1(OPTC) c2(XP_011507708) c3(opticin isoform X1) c4(297717, 297718) c5(297719, 297720, 297721, 297722, 297723, 297724, 297725, 297726, 297727, 297728, 297729, 297730, 297731, 297732, 297733, 297734) c6(297735, 297736, 297737, 297738) c7(HQ, Ew, W, pZ, dH, Be, cz); #c1(OPTN) c2(NP_001008214) c3(optineurin) c4(22869) c5(22870, 22871, 22872, 22873, 22874, 22875, 22876, 22877, 22878, 22879, 22880, 22881, 22882, 22883, 22884, 22885, 22886, 22887, 22888, 22889) c6( ) c7(cmC, HQ, cZ, T, zt, bbH, Vd, PJ, ad, zw, WY, cmS, aT, bdM, dL, vd, K, HR, dd, ae, eL, d, lR, dH, aW, wf, aiT, dw, cm, D, c, pZ, dB, Kv, U, EY, bi, oQ, lJ, cmR); #c1(OPTN) c2(XP_005252394) c3(optineurin isoform X1) c4(271690) c5(271691, 271692, 271693, 271694, 271695, 271696, 271697, 271698, 271699, 27/700) c6( ) c7(cmC, HQ, cZ, T, zt, bbH, Vd, PJ, ad, zw, WY, cmS, aT, bdM, dL, vd, K, HR, dd, ae, eL, d, lR, dH, aW, wf, aiT, dw, cm, D, c, pZ, dB, Kv, U, EY, bi, od, lJ, cmR); #c1(OPTN) c2(XP_005252395) c3(optineurin isoform X2) c4(271701, 271702) c5(271703, 271704, 271705, 271706, 271707, 271708, 271709, 27/710, 271711, 271712, 271713) c6( ) c7(cmC, HQ, cZ, T, zt, bbH, Vd, PJ, ad, zw, WY, cmS, aT, bdM, dL, vd, K, HR, dd, ae, eL, d, lR, dH, aW, wf, aiT, dw, cm, D, c, pZ, dB, Kv, U, EY, bi, oQ, lJ, cmR); #c1(OR10A2) c2(NP_001004460) c3(olfactory receptor 10A2) c4(20745) c5(20746, 20747, 20748, 20749, 20750, 20751) c6( ) c7(cmT); #c1(OR1DA4) c2(NP_997069) c3(olfactory receptor 10A4) c4(265019) c5(265020, 265021, 265022, 265023, 265024, 265025) c6( ) c7(bL, T, dD, eR, ea, e, aw, bO, aA, cz, n, cQ, aj, j, v, eq, aY, J, aE, ds, M, aT, oW, yu, dV, JK, bK, K, wb, h, uX, Nr, A, cW, aW, az, po, aF, hp, iV, qa, Nq, BU, mg, agf, cS, dy, lf); #c1(OR10C1) c2(NP_039229) c3(olfactory receptor 10C1) c4(176836, 176837, 176838, 176839, 176840, 176841, 176842) c5(176843, 176844, 176845, 176846, 176847, 176848, 176849, 176850, 176851, 176852, 176853, 176854, 176855, 176856, 176857, 176858, 176859, 176860, 176861, 176862, 176863, 176864, 176865, 176866, 176867, 176868, 176869, 176870, 176871, 176872, 176873, 176874, 176875, 176876, 176877, 176878, 176879, 176880, 176881, 176882, 176883, 176884, 176885, 176886, 176887, 176888, 176889, 176890, 176891, 176892, 176893, 176894, 176895, 176896, 176897, 176898, 176899, 176900, 176901, 176902, 176903, 176904, 176905, 176906, 176907, 176908) c6( ) c7(v, qQ, mg, cz); #c1(OR10J1) c2(NP_036483) c3(olfactory receptor 10J1) c4(174162) c5(174163, 174164, 174165, 174166, 174167, 174168) c6( ) c7(e, pN); #c1(OR10J3) c2(NP_001004467) c3(olfactory receptor 10J3) c4(20752) c5(20753, 20754, 20755, 20756, 20757, 20758) c6( ) c7(bL, T, A, aW, if, e, eZ, aT, eb, aj, v, qN, J); #c1(OR10J5) c2(NP_001004469) c3(olfactory receptor 10J5) c4(20759) c5(20760, 20761, 20762, 20763, 20764, 20765) c6( ) c7(bL, aZ, bO, Nt, cz, aj, ai, q, wD, J, ad, aT, alX, ae, aW, eZ, r, iV, wA, bAd, mg, ei, y); #c1(OR10K1) c2(NP_001004473) c3(olfactory receptor 10K1) c4(20783) c5(20784, 20785, 20786, 20787, 20788, 20789) c6( ) c7(bL, bM, T, dD, pf, ea, if, aw, aA, cz, bB, aj, j, eq, qN, ase, q, J, ad, dP, cF, l, bae, bNN, u, dV, aT, h, ae, s, aW, ff, eH, eZ, r, fe, aF, en, g, eF, bz, bE, cmU, bTe, mg, adg, y); #c1(OR10K2) c2(NP_001004476) c3(olfactory receptor 10K2) c4(20804) c5(20805, 20806, 20807, 20808, 20809, 20810) c6( ) c7(cT, mR, lX, alt, WY, aW, ag, hB, atj, bJL, aT, cz, eb, io, aw, cr, jh, aFV, eE); #c1(OR10R2) c2(NP_001004472) c3(olfactory receptor 10R2) c4(20776) c5(20777, 20778, 20779, 20780, 20781, 20782) c6( ) c7(bM, T, mr, aw, jh, cz, aj, cmV, Uy, J, hX, aT, vO, aW, Y, az, eZ, iV, eE, bz, Dy, ac, wx, R, y, ra, B); #c1(OR10T2) c2(NP_001004475) c3(olfactory receptor 1012) c4(20790, 20791) c5(20792, 20793, 20794, 20795, 20796, 20797, 20798, 20799, 20800, 20801, 20802, 20803) c6( ) c7(T, ajy, cF, A, cA, bE, aW, awN, NT, eZ, aT, bB, fe, j, fu, R, iV, eE, aYs, bA); #c1(OR10X1) c2(NP_001004477) c3(olfactory receptor 10X1) c4(20811) c5(20812, 20813, 20814, 20815, 20816, 20817, 20818, 20819, 20820, 20821, 20822) c6( ) c7(bL, bM, ahf, e, aw, cz, aj, fK, J, aE, aT, aOh, je, aW, Y, cr, eZ, fe, t, bz, pb, iM, ac, Jr, vy, m, y); #c1(OR10Z1) c2(NP_001004478) c3(olfactory receptor 10Z1) c4(20823) c5(20824, 20825, 20826, 20827, 20828, 20829) c6( ) c7(bL, T, iV, no, cF, ig, aW, Y, aT, fe, dG, dl, v, uE, ei, y, iK, eL, pb); #c1(OR11A1) c2(NP_039225) c3(olfactory receptor (11A1) c4(176748, 176749) c5(176750, 176751, 176752, 176753, 176754, 176755, 176756, 176757, 176758, 176759, 176760, 176761, 176762, 176763, 176764, 176765, 176766, 176767, 176768, 176769, 176770, 176771, 176772, 176773, 176774, 176775, 176776, 176777, 176778, 176779, 176780, 176781, 176782, 176783, 176784, 176785, 176786, 176787, 176788, 176789, 176790, 176791, 176792, 176793, 176794, 176795, 176796, 176797, 176798, 176799, 176800, 176801, 176802, 176803, 176804, 176805, 176806, 176807, 176808, 176809, 176810, 176811, 176812, 176813, 176814, 176815, 176816, 176817, 176818, 176819, 176820, 176821, 176822, 176823, 176824, 176825, 176826, 176827, 176828, 176829, 176830, 176831, 176832, 176833, 176834, 176835) c6( ) c7(eZ); #c1(OR12D2) c2(NP_039224) c3(olfactory receptor 1202) c4(176714, 176715, 176716, 176717) c5(176718, 176719, 176720, 176721, 176722, 176723, 176724, 176725, 176726, 176727, 176728, 176729, 176730, 176731, 176732, 176733, 176734, 176735, 176736, 176737, 176738, 176739, 176740, 176741, 176742, 176743, 176744, 176745, 176746, (76747) c6( ) c7(eZ); #c1(OR12D3) c2(NP_112221) c3(olfactory receptor (203) c4(220897, 220898) c5(220899, 220900, 220901, 220902, 220903, 220904, 220905, 220906, 220907, 220908, 220909, 220910, 220911, 220912, 220913, 220914, 220915, 220916, 220917, 220918, 220919, 220920, 220921, 220922, 220923, 220924, 220925, 220926, 220927, 220928, 220929, 220930, 220931, 220932, 220933, 220934, 220935, 220936, 220937, 220938, 220939, 220940, 220941, 220942, 220943, 220944, 220945, 220946, 220947, 220948, 220949) c6( ) c7(h, V, eZ); #c1(OR13C3) c2(NP_001001961) c3(olfactory receptor 13C3) c4(19216) c5(19217, 19218, 19219, 19220, 19221, 19222) c6( ) c7(aw, lb); #c1(OR13C4) c2(NP_001001919) c3(olfactory receptor 13C4) c4(19156, 19157) c5(19158, 19159, 19160, 19161, 19162, 19163, 19164, 19165, 19166, 19167, 19168, 19169) c6( ) c7(aC); #c1(OR13F1) c2(NP_001004485) c3(olfactory receptor 13F1) c4(20830) c5(20831, 20832, 20833, 20834, 20835, 20836) c6( ) c7(pu); #c1(OR13G1) c2(NP_001005487) c3(olfactory receptor 13G1) c4(21748) c5(21749, 21750, 21751, 21752, 21753, 21754) c6( ) c7(lD, bM, if, e, aw, NT, aA, uu, j, qN, ec, aT, eu, ly, mR, lR, aW, aF, pb, lx, ac, Ji, ei, ra); #c1(OR13 J1) c2(NP_001004487) c3(olfactory receptor 13J1) c4(20837) c5(20838, 20839, 20840, 20841, 20842, 20843) c6( ) c7(BU); #c1(OR14J1) c2(NP_112208) c3(olfactory receptor 14J1) c4(220818, 220819) c5(220820, 220821, 220822, 220823, 220824, 220825, 220826, 220827, 220828, 220829, 220830, 220831, 220832, 220833, 220834, 220835, 220836, 220837, 220838, 220839, 220840, 220841, 220842, 220843, 220844, 220845, 220846, 220847) c6( ) c7(A, eZ); #c1(OR1A1) c2(NP_055380) c3(olfactory receptor 1A1) c4(180687) c5(180688, 180689, 180690, 180691, 180692, 180693) c6( ) c7(aue); 1C1(OR1C1) c2(NP_036485) c3(olfactory receptor 1C1) c4(174169) c5(174170, 174171, 174172, 174173, 174/74, 174175) c6( ) c7(aE, ig, aW, WY, Y, aw, NT, eZ, aT, uu, ei, y, ae, B, ad); #c1(OR102) c2(NP_002539) c3(olfactory receptor 1D2) c4(129642) c5(129643, 129644, 129645, 129646, 129647, 129648) c6( ) c7(ci); #c1(DR1E1) c2(NP_003544) c3(olfactory receptor (E1) c4(138995) c5(138996, 138997, 138998, 138999, 139000, 139001) c6( ) c7(Kw); #c1(DR1E2) c2(NP_003545) c3(olfactory receptor 1E2) c4(139002, 139003) c5(139004, 139005, 139006, 139007, 139008, 139009, 139010, 139011, 139012, 139013, 139014, 139015) c6( ) c7(oe); #c1(OR1J2) c2(NP_473448) c3(olfactory receptor 1J2) c4(230525, 230526) c5(230527, 230528, 230529, 230530, 230531, 230532, 230533, 230534, 230535, 230536, 230537, 230538) c6( ) c7(cmW); #c1(OR1K1) c2(NP_543135) c3(olfactory receptor 1K1) c4(233341) c5(233342, 233343, 233344, 233345, 233346, 233347) c6( ) c7(Na, Nc); #c1(OR1L8) c2(NP_001004454) c3(olfactory receptor 1L8) c4(207/7) c5(20718, 20719, 20720, 20721, 20722, 20723) c6( ) c7(ac); #c1(OR1M1) c2(NP_001004456) c3(olfactory receptor (M1) c4(20724, 20725) c5(20726, 20727, 20728, 20729, 20730, 20731, 20732, 20733, 20734, 20735, 20736, 20737) c6( ) c7(y); #c1(OR1N1) c2(NP_036495) c3(olfactory receptor 1N1) c4(174/76) c5(174177, 174178, 174179, 174180, 174181, 174182) c6( ) c7(bL, T, aW, e, aw, ff, vy, aT, cz, eZ, aj, mf, en, iV, f, B, J, hm); #c1(OR1N2) c2(NP_001004457) c3(olfactory receptor 1N2) c4(20738) c5(20739, 20740, 20741, 20742, 20743, 20744) c6( ) c7(Na, Nc); #c1(OR2A25) c2(NP_001004488) c3(olfactory receptor 2A25) c4(20844) c5(20845, 20846, 20847, 20848, 20849, 20850) c6( ) c7(aw, eu, e, j, ac); #c1(OR2AG1) c2(NP_001004489) c3(olfactory receptor 2AG1) c4(20851) c5(20852, 20853, 20854, 20855, 20856, 20857) c6( ) c7(bL, T, if, bS, ct, qN, ai, aR, lg, iX, XY, aql, rE, he, bA, hZ, xi, Y, azP, cr, eZ, Z, FT, hq, ua, QT, y, ra, qc); #c1(OR2AK2) c2(NP_001004491) c3(olfactory receptor 2AK2) c4(20858) c5(20859, 20860, 20861, 20862, 20863, 20864) c6( ) c7(bL, bM, lD, eR, aT, cz, aj, j, aY, J, M, cmX, ly, s, dH, eZ, Mz, pb, lx, gk, ac, vy, dy, y, B); #c1(OR2AT4) c2(NP_001005285) c3(olfactory receptor 2AT4) c4(21308) c5(21309, 21310, 21311, 21312, 21313, 21314) c6( ) c7(fe); #c1(OR2B2) c2(NP_149046) c3(olfactory receptor 282) c4(226911) c5(226912, 226913, 226914, 226915, 226916, 226917) c6( ) c7(wA, iM, avP, wG, je, acz, tT, cr, aFr, v, pl, ai); #c1(OR2B3) c2(NP_001005226) c3 (putative olfactory receptor 283) c4(21208) c5(21209, 21210, 21211, 21212, 21213, 21214, 21215, 21216, 21217, 21218, 21219, 21220, 21221, 21222, 21223, 21224, 21225, 21226, 21227, 21228, 21229, 21230, 21231, 21232, 21233, 21234, 21235, 21236) c6( ) c7(aj, eZ, cr); #c1(OR2B6) c2(NP_036499) c3(olfactory receptor 286) c4(174183) c5(174184, 174185, 174186, 174187, 174188, 174189) c6( ) c7(v, mg, qQ, C, cz); #c1(OR2C1) c2(NP_036500) c3(olfactory receptor 2C1) c4(174190) c5(174191, 174192, 174193, 174194, 174195, 174196) c6( ) c7(oe); #c1(OR2F2) c2(NP_001004685) c3(olfactory receptor 2F2) c4(20865) c5(20866, 20867, 20868, 20869, 20870, 20871) c6( ) c7(fe, abW, j, SV, iV); #c1(OR2G2) c2(NP_001001915) c3(olfactory receptor 262) c4(19149) c5(19150, 19151, 19152, 19153, 19154, 19155) c6( ) c7(bL, T, Bv, B, bf, iV, hl, NT, r, sM, dL, j, pb, bt, eL, q, hq); #c1(OR2G3) c2(NP_001001914) c3 (olfactory receptor 263) c4(19142) c5(19143, 19144, 19145, 19146, 19147, 19148) c6( ) c7(bM, e, aT, aj, gf, j, ase, ai, J, ad, wD, WY, aT, ae, RT, ND, aW, Y, pb, hq, gk, Jr, y, B); #c1(OR2H2) c2(NP_009091) c3(olfactory receptor 2H2) c4(170625, 170626, 170627) c5(170628, 170629, 170630, 170631, 170632, 170633, 170634, 170635, 170636, 170637, 170638, 170639, 170640, 170641, 170642, 170643, 170644, 170645, 170646, 170647, 170648, 170649, 170650, 170651, 170652, 170653, 170654, 170655, 170656, 170657) c6( ) c7(jy, V, eZ); #c1(OR2J2) c2(NP_112167) c3(olfactory receptor 2J2) c4(220551, 220552, 220553, 220554, 220555) c5(220556, 220557, 220558, 220559, 220560, 220561, 220562, 220563, 220564, 220565, 220566, 220567, 220568, 220569, 220570, 220571, 220572, 220573, 220574, 220575, 220576, 220577, 220578, 220579, 220580, 220581, 220582, 220583, 220584, 220585, 220586, 220587, 220588, 220589, 220590, 220591, 220592, 220593, 220594, 220595, 220596, 220597, 220598, 220599, 220600, 220601) c6( ) c7(T, wA, eZ, j, ci, pl, eA, hm); #c1(OR2J3) c2(NP_001005216) c3(olfactory receptor 2J3) c4(21162, 21163, 21164) c5(21165, 21166, 21167, 21168, 21169, 21170, 21171, 21172, 21173, 21174, 21175, 21176, 21177, 21178, 21179, 21180, 21181, 21182, 21183, 21184, 21185, 21186, 21187, 21188, 21189, 21190, 21191, 21192, 21193, 21194, 21195, 21196, 21197) c6( ) c7(aW, cmY, eZ, aT); #c1(OR2L2) c2(NP_001004686) c3(olfactory receptor 2L2) c4(20872, 20873) c5(20874, 20875, 20876, 20877, 20878, 20879) c6( ) c7(bL, OW, eR, aW, M, aT, mg, fe, aj, j, qj, iV, aY, J, pC); #c1(OR2M3) c2(NP_001004689) c3 (olfactory receptor 2M3) c4(20880) c5(20881, 20882, 20883, 20884, 20885, 20886) c6( ) c7(mR, az, aA, cmZ, aF, hp, iV, y, Pr, fK); #c1(OR2M4) c2(NP_059974) c3(olfactory receptor 2M4) c4(193423) c5(193424, 193425, 193426, 193427, 193428, 193429) c6( ) c7(T, ac, dH, Y, aw, mg, cz, fe, mf, pT); #c1(OR2M7) c2(NP_001004691) c3(olfactory receptor 2M7) c4(20887) c5(20888, 20889, 20890, 20891, 20892, 20893) c6( ) c7(kT, aW, aLJ, aT, v, i); #c1(OR2S2) c2(NP_063950) c3(olfactory receptor 2S2) c4(202408) c5(202409, 202410, 202411, 202412, 202413, 202414) c6( ) c7(cF); #c1(OR2T1) c2(NP_112166) c3(olfactory receptor 211) c4(220544) c5(220545, 220546, 220547, 220548, 220549, 220550) c6( ) c7(bL, T, aW, eZ, aT, eb, aj, J); #c1(OR2T10) c2(NP_001004693) c3 (olfactory receptor 2110) c4(20901) c5(20902, 20903, 20904, 20905, 20906, 20907) c6( ) c7(bL, T, Bv, wD, cC, mx, lr, eZ, mf, j, aF, nA, uE, ZN, bz, pb, aoO); #c1(OR2T12) c2(NP_001004692) c3(olfactory receptor 2112) c4(20894) c5(20895, 20896, 20897, 20898, 20899, 20900) c6( ) c7(dP, x, s, aW, bW, eZ, r, aT, aA, cz, aF, j, k, ei, Qo, ZN, q, y); #c1(OR2T2) c2(NP_001004136) c3(olfactory receptor 212) c4( ) c5(20472, 20473, 20474, 20475, 20476) c6( ) c7(bL, bM, ac, eR, iW, eX, eZ, aA, Nt, alX, cz, aF, j, eV, aY, pb, qa); #c1(OR2T2) c2(XP_011545643) c3(olfactory receptor 212 isoform X1) c4(344582) c5(344583) c6( ) c7(bL, bM, ac, eR, iW, eX, eZ, aA, Nt, alX, cz, aF, j, eV, aY, pb, qa); #c1(OR2T33) c2(NP_001004695) c3(olfactory receptor 2133) c4(20908) c5(20909, 20910, 20911, 20912, 20913, 20914) c6( ) c7(bL, bM, ac, aik, ig, aZ, if, iR, cr, aA, eZ, aq, fo, aF, fe, ei, qN, pl, y, B); #c1 (OR2T4) c2(NP_001004696) c3(olfactory receptor 214) c4(20915) c5(20916, 20917, 20918, 20919, 20920, 20921) c6( ) c7(bL, aDs, bE, NT, eZ, bB, dy); #c1(OR2T5) c2(NP_001004697) c3(olfactory receptor 215) c4(20922) c5(20923, 20924, 20925, 20926, 20927, 20928) c6( ) c7(aw, ha, eZ, aA, bS, aF, ct, Z, he, y); #c1(OR2W1) c2(NP_112165) c3(olfactory receptor 2W1) c4(220516) c5(220517, 220518, 220519, 220520, 220521, 220522, 220523, 220524, 220525, 220526, 220527, 220528, 220529, 220530, 220531, 220532, 220533, 220534, 220535, 220536, 220537, 220538, 220539, 220540, 220541, 220542, 220543) c6( ) c7(eZ); #c1(OR2Y1) c2(NP_001001657) c3(olfactory receptor 2Y1) c4(18926) c5(18927, 18928, 18929, 18930, 18931, 18932) c6( ) c7(Gb, oW, afr, m, ra, t, Pr); #c1(OR2Z1) c2(NP_001004699) c3(olfactory receptor 221) c4(20929) c5(20930, 20931, 20932, 20933, 20934, 20935) c6( ) c7(gk, al); #c1(OR3A1) c2(NP_002541) c3(olfactory receptor 3A1) c4( ) c5(129649, 129650, 129651, 129652, 129653, 129654) c6( ) c7(al, cz, sM, ci, q, aR, alu, sO, aT, cy, dF, A, s, cH, aW, hl, r, aUP, t, gk, wE, vL, m, y, cE); #c1(OR3A1) c2(XP_011522196) c3(olfactory receptor 3A1 isoform X1) c4(316825) c5( ) c6( ) c7(al, cz, sM, ci, q, aR, aOu, sO, aT, cy, dF, A, s, cH, aW, hl, r, aUP, t, gk, wE, vL, m, y, cE); #c1(OR4A15) c2(NP_001005275) c3(olfactory receptor 4A15) c4(21273) c5(21274, 21275, 21276, 21277, 21278, 21279) c6( ) c7(eZ); #c1(OR4C12) c2(NP_001005270) c3(olfactory receptor 4C12) c4(21252) c5(21253, 21254, 21255, 21256, 21257, 21258) c6( ) c7(MU); #c1(OR4C13) c2(NP_001001955) c3(olfactory receptor 4C13) c4(19209) c5(19210, 19211, 19212, 19213, 19214, 19215) c6( ) c7(fr); #c1(OR4C6) c2(NP_001004704) c3(olfactory receptor 4C6) c4(20936, 20937) c5(20938, 20939, 20940, 20941, 20942, 20943, 20944, 20945, 20946, 20947, 20948, 20949) c6( ) c7(v); #c1(OR4D10) c2(NP_001004705) c3(olfactory receptor 4D10) c4(20950) c5(20951, 20952, 20953, 20954, 20955, 20956) c6( ) c7(bA); #c1(OR4K13) c2(NP_001004714) c3(olfactory receptor 4K13) c4(20957) c5(20958, 20959, 20960, 20961, 20962, 20963) c6( ) c7(bM); #c1(OR4P4) c2(NP_001004124) c3(olfactory receptor 4P4) c4(20434) c5(20435, 20436, 20437, 20438, 20439, 20440) c6( ) c7(v); #c1(OR4S2) c2(NP_001004059) c3(olfactory receptor 4S2) c4(20408) c5(20409, 20410, 20411, 20412, 20413, 20414) c6( ) c7(v); #c1(OR51A2) c2(NP_001004748) c3(olfactory receptor 51A2) c4(20987) c5(20988, 20989, 20990, 20991, 20992, 20993) c6( ) c7(fq); #c1(OR51A7) c2(NP_001004749) c3(olfactory receptor 51A7) c4(20994, 20995, 20996) c5(20997, 20998, 20999, 21000, 21001, 21002, 21003, 21004, 21005, 21006, 21007, 21008, 21009, 21010, 21011, 21012, 21013, 21014) c6( ) c7(j); #c1(OR51E1) c2(NP_689643) c3(olfactory receptor 51E1) c4(243404) c5(243405, 243406, 243407, 243408, 243409, 243410) c6( ) c7(ae, lB, oe, nG, ad); #c1(OR51E2) c2(NP_110401) c3(olfactory receptor 51E2) c4(220052, 220053) c5(220054, 220055, 220056, 220057, 220058, 220059, 220060, 220061, 220062, 220063, 220064, 220065) c6( ) c7(ae, ad); #c1(OR51F2) c2(NP_001004753) c3(olfactory receptor 51F2) c4(21015, 21016, 21017) c5(21018, 21019, 21020, 21021, 21022, 21023, 21024, 21025, 21026, 21027, 21028, 21029, 21030, 21031, 21032, 21033, 21034, 21035) c6( ) c7(Ea); #c1(OR51V1) c2(NP_001004760) c3(olfactory receptor 51V1) c4(21036) c5(21037, 21038, 21039, 21040, 21041, 21042) c6( ) c7(dV); #c1(OR52B2) c2(NP_001004052) c3(olfactory receptor 5282) c4(20380, 20381) c5(20382, 20383, 20384, 20385, 20386, 20387, 20388, 20389, 20390, 20391, 20392, 20393) c6( ) c7(aC); #c1(OR52B4) c2(NP_001005161) c3(olfactory receptor 5284) c4(21043) c5(21044, 21045, 21046, 21047, 21048, 21049) c6( ) c7(cF); #c1(OR52D1) c2(NP_001005163) c3(olfactory receptor 52D1) c4(21050) c5(21051, 21052, 21053, 21054, 21055, 21056) c6( ) c7(cF); #c1(OR52E6) c2(NP_001005167) c3(olfactory receptor 52E6) c4(21057, 21058) c5(21059, 21060, 21061, 21062, 21063, 21064, 21065, 21066, 21067, 21068, 21069, 21070) c6( ) c7(cF); #c1(OR52I1) c2(NP_001005169) c3(olfactory receptor 52I1) c4(21071, 21072) c5(21073, 21074, 21075, 21076, 21077, 21078, 21079, 21080, 21081, 21082, 21083, 21084) c6( ) c7(bL); #c1(OR52I2) c2(NP_001005170) c3 (olfactory receptor 52I2) c4(21085) c5(21086, 21087, 21088, 21089, 21090, 21091) c6( ) c7(bvS, aF, dl, aA); #c1(OR52K1) c2(NP_001005171) c3(olfactory receptor 52K1) c4(21092) c5(21093, 21094, 21095, 21096, 21097, 21098) c6( ) c7(gV, j, eZ, azW, hG); #c1(OR52K2) c2(NP_001005172) c3 (olfactory receptor 52K2) c4(21099) c5(21100, 21101, 21102, 21103, 21104, 21105) c6( ) c7(y, cl, qW, o); #c1(OR52M1) c2(NP_001004137) c3(olfactory receptor 52M1) c4(20477) c5(20478, 20479, 20480, 20481, 20482, 20483) c6( ) c7(s); #c1(OR5D18) c2(NP_001001952) c3(olfactory receptor 5018) c4(19200) c5(19201, 19202, 19203, 19204, 19205, 19206, 19207, 19208) c6( ) c7(aw); #c1(OR5H2) c2(NP_001005482) c3(olfactory receptor 5H2) c4(21741) c5(21742, 21743, 21744, 21745, 21746, 21747) c6( ) c7(bc); #c1(OR5H6) c2(NP_001005479) c3(olfactory receptor 5H6) c4(21734) c5(21735, 21736, 21737, 21738, 21739, 21740) c6( ) c7(aW, ds, LZ, aT, v, ra); #c1(OR5K1) c2(NP_001004736) c3(olfactory receptor 5K1) c4(20973) c5(20974, 20975, 20976, 20977, 20978, 20979) c6( ) c7(T, pu, AL, dN, cl, cna, dy, k, o); #c1(OR5K2) c2(NP_001004737) c3(olfactory receptor 5K2) c4(20980) c5(20981, 20982, 20983, 20984, 20985, 20986) c6( ) c7(bM, xN, Bv, cT, lX, lh, cz, v, tr, kU, y, wD); #c1(OR5V1) c2(NP_110503) c3(olfactory receptor 5V1) c4(220453) c5(220454, 220455, 220456, 220457, 220458, 220459, 220460, 220461, 220462, 220463, 220464, 220465, 220466, 220467, 220468, 220469, 220470, 220471, 220472, 220473, 220474, 220475, 220476, 220477, 220478, 220479, 220480, 220481, 220482, 220483, 220484, 220485, 220486, 220487, 220488, 220489, 220490, 220491) c6( ) c7(h, eZ); #c1(OR5V1) c2(XP_011546413) c3(olfactory receptor 5V1 isoform X1) c4(345170) c5(345171, 345172, 345173, 345174, 345175, 345176, 345177, 345178, 345179, 345180) c6( ) c7(h, eZ); #c1(OR5V1) c2(XP_011546799) c3(olfactory receptor 5V1 isoform X1) c4(345685) c5(345686, 345687, 345688, 345689, 345690, 345691, 345692, 345693, 345694, 345695, 345696, 345697, 345698, 345699) c6( ) c7(h, eZ); #c1(OR6B1) c2(NP_001005281) c3(olfactory receptor 681) c4(21294) c5(21295, 21296, 21297, 21298, 21299, 21300) c6( ) c7(oO, l, A, u, pf, aT, fd, bS, ct, lS, Z, cnb); #c1(OR6B2) c2(NP_001005853) c3(olfactory receptor 682) c4(22123) c5(22124, 22125, 22126, 22127, 22128) c6( ) c7(bM, T, ahf, mr, if, ht, al, aj, qN, q, J, dV, aT, hu, kU, db, aa, s, amy, aW, lh, ff, r, fe, ls, vs, en, g, eE, wA, bz, gk, ac, lp, ag, cmU, Be, ra, B); #c1(OR6B2) c2(XP_005247061) c3 (olfactory receptor 682 isoform X1) c4( ) c5(267636) c6( ) c7(bM, T, ahf, mr, if, ht, al, aj, qN, q, J, dV, aT, hu, KU, db, aa, s, amy, aW, lh, ff, r, fe, ls, vs, en, g, eE, wA, bz, gk, ac, lp, ag, cmU, Be, ra, B); #c1(OR6C1) c2(NP_001005182) c3(olfactory receptor 6C1) c4(21113) c5(2/114, 21115, 21116, 21117, 21118, 21119) c6( ) c7(cF); #c1(OR6F1) c2(NP_001005286) c3(olfactory receptor 6F1) c4(21315) c5(21316, 21317, 21318, 21319, 21320, 21321) c6( ) c7(bL, lD, xN, aZ, if, eb, bB, aj, j, qj, qN, aY, q, J, aT, ly, mR, s, aW, r, iV, pb, lx, bE, dy, ei, y, bLi, pC); #c1(OR6K2) c2(NP_001005279) c3(olfactory receptor 6K2) c4(21287) c5(21288, 21289, 21290, 21291, 21292, 21293) c6( ) c7(bL, T, ac, s, aW, wE, aw, aT, sM, hD, vs, aS, j, y, bz); #c1(OR6K3) c2(NP_001005327) c3 (olfactory receptor 6K3) c4(21338, 21339) c5(21340, 21341, 21342, 21343, 21344, 21345, 21346, 21347, 21348, 21349, 21350, 21351) c6( ) c7(iV, ahf, ac, aW, if, M, r, aT, fK, cz, mg, aj, hD, qN, pb, fe, y, bz, q, J, wD); #c1(OR6K6) c2(NP_001005184) c3(olfactory receptor 6K6) c4(21120) c5(21121, 21122, 21123, 21124, 21125, 21126) c6( ) c7(kT, T, w, wD, HJ, ig, QU, aoO, eZ, fe, aj, v, aF, iV, y, bz, J, hm); #c1(OR6N1) c2(NP_001005185) c3(olfactory receptor GN1) c4(21127) c5(21128, 21129, 21130, 21131, 21132, 21133) c6( ) c7(bL, c, aW, aT, aj, v, ei, y, J, us); #c1(OR6N2) c2(NP_001005278) c3(olfactory receptor 6N2) c4(21280) c5(21281, 21282, 21283, 21284, 21285, 21286) c6( ) c7(bL, aW, ag, ajd, aT, gf, y, pb, hq); #c1(OR6X1) c2(NP_001005188) c3(olfactory receptor 6X1) c4(21134) c5(21135, 21136, 21137, 21138, 21139, 21140) c6( ) c7(ac); #c1(OR6Y1) c2(NP_001005189) c3 (olfactory receptor 6Y1) c4(21141) c5(21142, 21143, 21144, 21145, 21146, 21147) c6( ) c7(bL, kT, y, aW, aT, aj, bS, bK, m, ct, j, Z, t, pb, J); #c1(OR7C1) c2(NP_945182) c3(olfactory receptor 7C1) c4( ) c5(260699, 260700, 260701, 260702, 260703) c6( ) c7(y); #c1(OR7C1) c2(XP_011526217) c3 (olfactory receptor 7C1 isoform X2) c4(320478) c5(320479) c6( ) c7(y); #c1(OR7D2) c2(NP_787079) c3 (olfactory receptor 702) c4(252083) c5(252084, 252085, 252086, 252087, 252088, 252089) c6( ) c7(gk, al); #c1(OR7E24) c2(NP_001073404) c3(olfactory receptor 7E24) c4(35998) c5(35999, 36000, 36001, 36002, 36003, 36004) c6( ) c7(cd, cz); #c1(OR6S1) c2(NP_001005203) c3(olfactory receptor 8S1) c4(21148) c5(21149, 21150, 21151, 21152, 21153, 21154) c6( ) c7(cF); #c1(OR9K2) c2(NP_001005243) c3(olfactory receptor 9K2) c4(21245) c5(21246, 21247, 21248, 21249, 21250, 21251) c6( ) c7(bM, cT, s); #c1(OR9Q2) c2(NP_001005283) c3(olfactory receptor 902) c4(21301) c5(21302, 21303, 21304, 21305, 21306, 21307) c6( ) c7(EQ); #c1(ORAI1) c2(NP_116 (79) c3(calcium release-activated calcium channel protein 1) c4(225621) c5(225622, 225623, 225624, 225625, 225626, 225627) c6( ) c7(T, hl, dA, cz, n, ad, bjv, aT, h, ae, d, mR, aW, Y, oV, xs, lo, cnc, eE, aVc, cnd, im, lf); #c1(ORAI3) c2(NP_689501) c3 (protein orai-3) c4(242947, 242948) c5(242949, 242950, 242951, 242952, 242953, 242954, 242955, 242956, 242957, 242958, 242959, 242960) c6( ) c7(T, eM, aW, aT, ae, iu, ad); #c1(ORAOV1) c2(NP_703152) c3(oral cancer-overexpressed protein 1) c4(246051, 246052) c5(246053, 246054, 246055, 246056, 246057, 246058, 246059, 246060, 246061, 246062, 246063, 246064, 246065, 246066, 246067, 246068) c6( ) c7(T, if, ff, K, qN, en); #c1(ORAOV1) c2(XP_011543148) c3(oral cancer-overexpressed protein 1 isoform X3) c4(341971) c5(341972, 341973, 341974, 341975, 341976, 341977) c6( ) c7(T, if, ff, K, qN, en); #c1(ORC2) c2(NP_006181) c3(origin recognition complex subunit 2) c4(162637, 162638) c5(162639, 162640, 162641, 162642, 162643, 162644, 162645, 162646, 162647, 162648, 162649, 162650, 162651, 162652, 162653, 162654, 162655, 162656) c6( ) c7(py); #c1(ORC2) c2(XP_011509556) c3(origin recognition complex subunit 2 isoform X2) c4(300135) c5( ) c6( ) c7(py); #c1(DRC3) c2(NP_001184188) c3(origin recognition complex subunit 3 isoform 3) c4( ) c5(75552, 75553, 75554, 75555, 75556) c6( ) c7(d, aaU, z, cj, h, ae, eW, ad); #c1(ORC3) c2(NP_036513) c3(origin recognition complex subunit 3 isoform 2) c4(174197, (74198) c5(174199, 174200, 174201, 174202, 174203, 174204, 174205) c6( ) c7(d, aaU, z, cj, h, ae, eW, ad); #c1(ORC3) c2(NP_862820) c3(origin recognition complex subunit 3 isoform 1) c4(255749) c5(255750, 255751, 255752, 255753, 255754, 255755, 255756) c6( ) c7(d, aaU, z, cj, h, ae, eW, ad); #c1(ORC3) c2(XP_005248763) c3(origin recognition complex subunit 3 isoform X3) c4(268561) c5( ) c6( ) c7(d, aaU, z, cj, h, ae, eW, ad); #c1(DRC3) c2(XP_011533954) c3(origin recognition complex subunit 3 isoform X4) c4( ) c5(330154, 330155, 330156, 330157, 330158) c6( ) c7(d, aaU, z, cj, h, ae, eW, ad); #c1(ORC4) c2(NP_001177810) c3(origin recognition complex subunit 4 isoform 2) c4( ) c5(73058, 73059, 73060, 73061, 73062, 73063, 73064, 73065, 73066, 73067, 73068) c6( ) c7(cne, ali, cj); #c1(ORC4) c2(NP_001177811) c3(origin recognition complex subunit 4 isoform 3) c4( ) c5(73069, 73070, 73071, 73072, 73073, 73074) c6( ) c7(cne, ali, cj); #c1(ORC4) c2(NP_002543) c3(origin recognition complex subunit 4 isoform 1) c4(129655, 129656, 129657, 129658, 129659, 129660, 129661) c5(129662, 129663, 129664, 129665, 129666, 129667, 129668, 129669, 129670, 129671, 129672, 129673, 129674, 129675, 129676, 129677, 129678, 129679, 129680, 129681, 129682, 129683, 129684, 129685, 129686, 129687, 129688, 129689, 129690, 129691, 129692, 129693, 129694, 129695, 129696, 129697, 129698) c6( ) c7(cne, ali, cj); #c1(ORC5) c2(NP_002544) c3(origin recognition complex subunit 5 isoform 1) c4(129699, 129700) c5(129701, 129702, 129703, 129704, 129705, 129706, 129707, 129708, 129709, 129710, 129711, 129712) c6( ) c7(T, cF, iT, vA, bw, bt, op); #c1(ORC5) c2(NP_859531) c3(origin recognition complex subunit 5 isoform 2) c4(255550) c5(255551, 255552, 255553, 255554, 255555, 255556) c6( ) c7(T, cF, iT, vA, bw, bt, op); #c1(ORC6) c2(NP_055136) c3(origin recognition complex subunit 6) c4(179213) c5(179214, 179215, 179216, 179217, 179218, 179219) c6( ) c7(T, bE, r, bB, ali, q, X, cnf); #c1(ORM1) c2(NP_000598) c3(alpha-1-acid glycoprotein 1 precursor) c4(13634) c5(13635, 13636, 13637, 13638, 13639, 13640, 13641, 13642, 13643, 13644, 13645, 13646, 13647, 13648, 13649, 13650, 13651) c6( ) c7(bM, T, A, cA, iU, fO, bS, v, ct, ci, pM, Z, fb, aaD, uQ); #c1(ORM2) c2(NP_000599) c3(alpha-l-acid glycoprotein 2 precursor) c4(13652, 13653, 13654) c5(13655, 13656, 13657, 13658, 13659, 13660, 13661) c6( ) c7(Ob, aaD, iU, r); #c1(ORMDL3) c2(XP_005257884) c3(ORM1-like protein 3 isoform X1) c4(275399) c5(275400, 275401, 275402, 275403, 275404, 275405, 275406, 275407, 275408, 275409) c6( ) c7(bM, iV, A, mR, z, LX, sp, JM, at, v, K, pM, y, eA, JB); #c1(OS9) c2(NP_001017956) c3 (protein OS-9 isoform 2 precursor) c4(26630) c5(26631, 26632, 26633, 26634, 26635, 26636) c6( ) c7(bL, A, Y, cz, j, y); #c1(DS9) c2(NP_001017957) c3(protein OS-9 isoform 3 precursor) c4(26637) c5(26638, 26639, 26640, 26641, 26642, 26643) c6( ) c7(bL, A, Y, cz, j, y); #c1(OS9) c2(NP_001017958) c3(protein OS-9 isoform 4 precursor) c4(26644) c5(26645, 26646, 26647, 26648, 26649, 26650) c6( ) c7(bL, A, Y, cz, j, y); #c1(OS9) c2(NP_001248349) c3(protein OS-9 isoform 5 precursor) c4(93742) c5(93743, 93744, 93745, 93746, 93747, 93748) c6( ) c7(bL, A, Y, cz, j, y); #c1(DS9) c2(NP_001248350) c3(protein OS-9 isoform 6 precursor) c4(93749) c5(93750, 93751, 93752, 93753, 93754, 93755) c6( ) c7(bL, A, Y, cz, j, y); #c1(OS9) c2(NP_001248351) c3(protein OS-9 isoform 7 precursor) c4(93756) c5(93757, 93758, 93759, 93760, 93761, 93762) c6( ) c7(bL, A, Y, cz, j, y); #c1(OS9) c2(NP_006803) c3(protein OS-9 isoform 1 precursor) c4(168238) c5(168239, 168240, 168241, 168242, 168243, 168244) c6( ) c7(bL, A, Y, cz, j, y); #c1(OSBP) c2(NP_002547) c3(oxysterol-binding protein 1) c4(129713, 129714) c5(129715, 129716, 129717, 129718, 129719, 129720, 129721, 129722, 129723, 129724, 129725, 129726) c6( ) c7(Fi, T, OV, aE, ei); #c1(OSBP2) c2(NP_001269667) c3(oxysterol-binding protein 2 isoform c) c4(102474) c5(102475, 102476, 102477, 102478, 102479, 102480, 102481, 102482, 102483, 102484, 102485, 102486) c6( ) c7(W, iU); #c1(OSBP2) c2(NP_001269668) c3(oxysterol-binding protein 2 isoform d) c4(102487) c5( ) c6( ) c7(W, iU); #c1(OSBP2) c2(NP_001269670) c3(oxysterol-binding protein 2 isoform f) c4(102488) c5(102489, 102490, 102491, 102492, 102493, 102494) c6( ) c7(W, iU); #c1(OSBP2) c2(NP_110385) c3(oxysterol-binding protein 2 isoform a) c4(219850) c5(219851, 219852, 219853, 219854, 219855, 219856) c6( ) c7(W, iU); #c1(OSBP2) c2(XP_005261522) c3(oxysterol-binding protein 2 isoform X3) c4(277467) c5(277468, 277469, 277470, 277471, 277472, 277473) c6( ) c7(W, iU); #c1(OSBPL10) c2(NP_001167531) c3(oxysterol-binding protein-related protein 10 isoform 2) c4(69001) c5(69002, 69003, 69004, 69005, 69006, 69007) c6( ) c7(af, Fi, aq, aS, ei, dQ, B); #c1(OSBPL10) c2(NP_060254) c3(oxysterol-binding protein-related protein 10 isoform 1) c4(195041) c5(195042, 195043, 195044, 195045, 195046, 195047) c6( ) c7(af, Fi, aq, aS, ei, dQ, B); #c1(OSBPL11) c2(NP_073613) c3(oxysterol-binding protein-related protein 11) c4(213139) c5(213140, 213141, 213142, 213143, 213144, 213145) c6( ) c7(e, aj, v, ey, ei, J); #c1(OSBPL1A) c2(NP_060500) c3(oxysterol-binding protein-related protein 1 isoform 1) c4( ) c5(196257, 196258, 196259, 196260, 196261) c6( ) c7(cm, v, bA); #c1(OSBPL1A) c2(NP_542164) c3(oxysterol-binding protein-related protein 1 isoform 2) c4(231484, 231485, 231486) c5(231487, 231488, 231489, 231490, 231491, 231492, 231493, 231494, 231495, 231496, 231497, 231498, 231499) c6( ) c7(cm, v, bA); #c1(OSBPL1A) c2(XP_006722444) c3(oxysterol-binding protein-related protein 1 isoform X2) c4(294712) c5( ) c6( ) c7(cm, v, bA); #c1(OSBPL1A) c2(XP_011524102) c3(oxysterol-binding protein-related protein 1 isoform X5) c4( ) c5(318668, 318669, 318670, 318671, 318672, 318673) c6( ) c7(cm, v, bA); #c1(OSBPL2) c2(NP_001265578) c3(oxysterol-binding protein-related protein 2 isoform 3) c4(98875) c5(98876, 98877, 98878, 98879, 98880, 98881, 98882, 98883, 98884, 98885, 98886, 98887, 98888, 98889, 98890, 98891, 98892) c6( ) c7(bqJ); #c1(OSBPL2) c2(NP_055650) c3(oxysterol-binding protein-related protein 2 isoform 1) c4(182539) c5(182540, 182541, 182542, 182543, 182544, 182545) c6( ) c7(bqJ); #c1(OSBPL2) c2(NP_653081) c3(oxysterol-binding protein-related protein 2 isoform 2) c4(238831, 238832) c5(238833, 238834, 238835, 238836, 238837, 238838, 238839) c6( ) c7(bqJ); #c1(OSBPL2) c2(XP_011527420) c3(oxysterol-binding protein-related protein 2 isoform X1) c4(322156) c5( ) c6( ) c7(bqJ); #c1(OSBPL3) c2(NP_056365) c3(oxysterol-binding protein-related protein 3 isoform a) c4(187320) c5(187321, 187322, 187323, 187324, 187325, 187326) c6( ) c7(ey, Hr); #c1(OSBPL3) c2(NP_663160) c3(oxysterol-binding protein-related protein 3 isoform b) c4(241103, 241104, 241105, 241106) c5(241107, 241108, 241109, 241110, 24/111, 241112, 241113, 241114, 241115, 241116) c6( ) c7(By, Hr); #c1(OSBPL3) c2(NP_663161) c3(oxysterol-binding protein-related protein 3 isoform c) c4(241117) c5(241118, 241119, 241120, 241121, 241122, 24/123, 241124, 241125, 241126, 241127) c6( ) c7(ey, Hr); #c1(DSBPL3) c2(NP_663162) c3(oxysterol-binding protein-related protein 3 isoform d) c4(241128) c5(241129, 241130, 241131, 241132, 241133, 241134, 241135, 241136, 241137, 241138, 241139, 241140, 241141, 241142, 241143, 241144, 241145, 241146, 241147, 241148, 241149, 241150, 241151, 241152, 241153, 241154, 241155, 241156) c6( ) c7(ey, Hr); #c1(OSBPL5) c2(NP_065947) c3(oxysterol-binding protein-related protein 5 isoform a) c4(206400, 206401, 206402) c5(206403, 206404, 206405, 206406, 206407, 206408, 206409, 206410, 206411, 206412, 206413, 206414, 206415, 206416, 206417, 206418, 206419, 206420, 206421) c6( ) c7(T, aC, aE); #c1(OSBPL5) c2(NP_663613) c3(oxysterol-binding protein-related protein 5 isoform b) c4(241265, 241266, 241267, 241268, 241269, 241270, 24/271) c5(241272, 241273, 241274, 241275, 241276, 241277, 241278, 241279, 241280, 241281, 241282, 241283, 241284, 241285, 241286, 241287, 241288, 241289, 241290, 241291, 241292, 241293, 241294, 241295, 241296, 241297, 241298, 241299, 241300, 241301, 241302, 241303, 241304, 241305, 241306, 241307, 241308, 241309, 241310, 241311, 241312, 241313, 241314, 241315, 241316) c6( ) c7(T, aC, aE); #c1(OSBPL6) c2(NP_001188409) c3(oxysterol-binding protein-related protein 6 isoform c) c4(79153) c5(79154, 79155, 79156, 79157, 79158, 79159) c6( ) c7(bN, bX); #c1(OSBPL6) c2(NP_001188410) c3(oxysterol-binding protein-related protein 6 isoform d) c4(79160) c5(79161, 79162, 79163, 79164, 79165, 79166) c6( ) c7(bN, bX); #c1(DSBPL6) c2(NP_001188411) c3(oxysterol-binding protein-related protein 6 isoform e) c4(79167) c5(79168, 79169, 79170, 79171, 79172, 79173, 79174, 79175, 79176, 79177, 79178, 79179) c6( ) c7(bN, bX); #c1(OSBPL6) c2(NP_115912) c3(oxysterol-binding protein-related protein 6 isoform a) c4(224643, 224644) c5(224645, 224646, 224647, 224648, 224649, 224650) c6( ) c7(bN, bX); #c1(OSBPL6) c2(NP_665682) c3(oxysterol-binding protein-related protein 6 isoform b) c4(241645) c5(241646, 241647, 241648, 241649, 241650, 241651) c6( ) c7(bN, bX); #c1(OSBPL8) c2(NP_001003712) c3(oxysterol-binding protein-related protein 8 isoform b) c4(20008) c5(20009, 20010, 20011, 20012, 20013, 20014, 20015, 20016, 20017, 20018, 20019, 20020, 20021, 20022, 20023, 20024, 20025, 20026) c6( ) c7(dD, eq); #c1(OSBPL8) c2(NP_065892) c3(oxysterol-binding protein-related protein 8 isoform a) c4(206086, 206087, 206088, 206089) c5(206090, 206091, 206092, 206093, 206094, 206095, 206096, 206097, 206098, 206099, 206100, 206101, 206102, 206103, 206104, 206105, 206106, 206107, 206108, 206109, 206110, 206111, 206112, 206113, 206114, 206115, 206116) c6( ) c7(dD, eq); #c1(OSBPL8) c2(XP_005268679) c3(oxysterol-binding protein-related protein 8 isoform X5) c4(282046) c5(282047, 282048, 282049, 282050, 282051, 282052) c6( ) c7(dD, eq); #c1(OSBPL8) c2(XP_011536159) c3(oxysterol-binding protein-related protein 8 isoform X4) c4(333474) c5( ) c6( ) c7(dD, eq); #c1(OSBPL8) c2(XP_011536160) c3(oxysterol-binding protein-related protein 8 isoform X6) c4(333475) c5(333476) c6( ) c7(dD, eq); #c1(OSBPL9) c2(XP_011538901) c3(oxysterol-binding protein-related protein 9 isoform X2) c4( ) c5(336968, 336969, 336970, 336971, 336972) c6( ) c7(fq, bqJ); #c1(OSBPL9) c2(XP_011538904) c3(oxysterol-binding protein-related protein 9 isoform X6) c4(336973) c5( ) c6( ) c7(fq, bqJ); #c1(OSBPL9) c2(XP_011538907) c3(oxysterol-binding protein-related protein 9 isoform X10) c4( ) c5(336974) c6( ) c7(fq, bqJ); #c1(OSCAR) c2(NP_001269278) c3(osteoclast-associated immunoglobulin-like receptor isoform 6 precursor) c4(101026) c5(101027, 101028, 101029, 101030, 101031, 101032, 101033) c6( ) c7(ea, aj, dD, ai, eq); #c1(OSCAR) c2(NP_573398) c3(osteoclast-associated immunoglobulin-like receptor isoform 5 precursor) c4(234467) c5(234468, 234469, 234470, 234471, 234472, 234473, 234474) c6( ) c7(ea, aj, dD, ai, eq); #c1(OSCAR) c2(NP_573399) c3(osteoclast-associated immunoglobulin-like receptor isoform 4 precursor) c4(234475) c5(234476, 234477, 234478, 234479, 234480, 234481, 234482) c6( ) c7(ea, aj, dD, ai, eq); #c1(OSCAR) c2(XP_011545812) c3(osteoclast-associated immunoglobulin-like receptor isoform X3) c4(344681) c5(344682, 344683, 344684, 344685, 344686, 344687, 344688) c6( ) c7(ea, aj, dD, ai, eq); #c1(OSCAR) c2(XP_011545813) c3(osteoclast-associated immunoglobulin-like receptor isoform X4) c4(344689) c5(344690, 344691, 344692, 344693, 344694, 344695, 344696, 344697, 344698, 344699) c6( ) c7(ea, aj, dD, ai, eq); #c1(OSER1) c2(XP_011527155) c3(oxidative stress-responsive serine-rich protein 1 isoform X1) c4(321663) c5(321664, 321665, 321666, 321667, 321668, 321669, 321670, 321671, 321672, 321673) c6( ) c7(ff, cz, aA, jy, aOM, aF, K, en, er, qa, ad); #c1(OSGEP) c2(NP_060277) c3(probable tRNA NG-adenosine threonylcarbamoyltransferase) c4(195174) c5(195175, 195176, 195177, 195178, 195179, 195180) c6( ) c7(alf, qs, dy, bX, cz); #c1(OSG1N1) c2(NP_892026) c3(oxidative stress-induced growth inhibitor 1) c4(257642, 257643) c5(257644, 257645, 257646, 257647, 257648, 257649, 257650, 257651, 257652, 257653, 257654, 257655, 257656, 257657, 257658, 257659) c6( ) c7(es, T, ac, iA, aW, aT, aq, K, iR, B); #c1(OSM) c2(NP_065391) c3(oncostatin-M preproprotein) c4(204690, 204691) c5(204692, 204693, 204694, 204695, 204696, 204697, 204698, 204699, 204700) c6( ) c7(oO, bL, T, Ob, FR, lT, aw, al, aDT, jh, fd, bB, cL, v, ci, V, cd, o, ad, aDl, ai, eA, Mo, iu, hb, rw, aT, ji, K, de, ae, cp, hm, hM, yo, d, Di, s, qt, aW, Y, nO, cr, fQ, dq, aq, fe, as, pl, eE, cm, yR, ck, uP, gk, dB, bE, mx, cj, Nl, uz, aS, Hb, m, fD, y, B, cn); #c1(OSM) c2(XP_011528504) c3(oncostatin-M isoform X1) c4( ) c5(323609, 323610, 323611, 323612, 323613, 323614, 323615) c6( ) c7(oO, bL, T, Ob, FR, lT, aw, aT, aDT, jh, fd, bB, cL, v, ci, V, cd, o, ad, aDl, ai, eA, Mo, iu, hb, rw, aT, ji, K, dQ, ae, cp, hm, hM, yo, d, Di, s, qt, aW, Y, nO, cr, fQ, dq, aq, fe, as, pl, eE, cm, yR, ck, uP, gk, dB, bE, mx, cj, Nl, uz, aS, Hb, m, fD, y, B, cn); #c1(OSMR) c2(NP_001161827) c3(oncostatin-M-specific receptor subunit beta isoform 2 precursor) c4(66107) c5(66108, 66109, 66110, 66/11, 66112, 66113) c6( ) c7(T, cng, eM, bE, aW, vj, cn, aT, bB, n, h, eZ, ey, m, ci, aay, g, t, it, y, cp); #c1(OSMR) c2(NP_003990) c3(oncostatin-M-specific receptor subunit beta isoform 1 precursor) c4(142681) c5(142682, 142683, 142684, 142685, 142686, 142687) c6( ) c7(T, cng, eM, bE, aW, vj, cn, aT, bB, n, h, eZ, ey, m, ci, aay, g, t, it, y, cp); #c1(OSR1) c2(XP_0067 (2005) c3(protein odd-skipped-related 1 isoform X1) c4(286324) c5(286325, 286326, 286327, 286328, 286329, 286330, 286331, 286332, 286333, 286334, 286335) c6( ) c7(qa, ac, ff, aA, cz, jy, aOM, Jd, aF, K, en, avG, er, fq, ad); #c1(OSR2) c2(NP_001135934) c3(protein odd-skipped-related 2 isoform a) c4( ) c5(53604, 53605, 53606, 53607, 53608, 53609, 53610, 53611) c6( ) c7(dP, Na); #c1(OSR2) c2(NP_001273770) c3(protein odd-skipped-related 2 isoform c) c4(105857, 105858) c5(105859, 105860, 105861, 105862, 105863, 105864) c6( ) c7(dP, Na); #c1(OSR2) c2(NP_443727) c3(protein odd-skipped-related 2 isoform b) c4(230024) c5(230025, 230026, 230027, 230028, 230029, 230030, 230031, 230032, 230033, 230034) c6( ) c7(dP, Na); #c1(OSR2) c2(XP_005250835) c3(protein odd-skipped-related 2 isoform X2) c4(270603) c5(270604, 270605) c6( ) c7(dP, Na); #c1(OSR2) c2(XP_011515127) c3(protein odd-skipped-related 2 isoform X3) c4( ) c5(307985) c6( ) c7(dP, Na); #c1(OSR2) c2(XP_011515128) c3(protein odd-skipped-related 2 isoform X4) c4( ) c5(307986) c6( ) c7(dP, Na); #c1(OSTF1) c2(NP_036515) c3(osteoclast-stimulating factor 1) c4(174206) c5(174207, 174208, 174209, 174210, 174211, 174212) c6( ) c7(FE, V, fq, j, af); #c1(OSTM1) c2(NP_054747) c3(osteopetrosis-associated transmembrane protein 1) c4(177559, 177560) c5(177561, 177562, 177563, 177564, 177565, 177566, 177567, 177568, 177569, 177570, 177571, (77572) c6( ) c7(zs, cnh, Lq, ajW, cni); #c1(OTC) c2(NP_000522) c3(ornithine carbamoyltransferase, mitochondrial precursor) c4(11689, 11690, 11691, 11692, 11693, 11694, 11695) c5(11696, 11697, 11698, 11699, 11700, 11701, 11702, 11703, 11704, 11705, 11706, 11707, 11708, 11709, 11710, 11711, 11712, 11713, 11714, 11715, 11716, 11717, 11718, 11719, 11720, 11721, 11722, 11723, 11724, 11725, 11726, 11727, 11728, 11729, 11730, 11731, 11732, 11733, 11734, 11735, 11736, 11737, 11738, 11739, 11740, 11741, 11742, 11743, 11744, 11745, 11746, 11747, 11748, 11749, 11750, 11751, 11752, 11753, 11754, 11755, 11756, 11757, 11758, 11759, 11760, 11761, 11762, 11763, 11764, 11765, 11766, 11767, 11768, 11769, 11770, 11771, 11772, 11773, 11774, 11775, 11776, 11777, 11778, 11779, 11780, 11781, 11782, 11783, 11784, 11785, 11786, 11787, 11788, 11789, 11790, 11791, 11792) c6(11793, 11794, 11795) c7(gx, ahW, e, aip, Ri, n, cz, cnk, v, jA, C, afD, HU, bfD, afR, hb, lG, ayA, jo, bq, ais, af, UF, cnl, bqy, xs, fc, fQ, dq, uY, BG, Ku, Pd, hJ, nC, KM, ayj, dr, ar, mO, iO, cnj, eB, Be, aUS, Uw, B, jr); #c1(OTOA) c2(NP_001155155) c3(otoancorin isoform 3) c4(61485) c5(61486, 61487, 61488, 61489, 61490, 61491) c6( ) c7(c, alA, com); #c1(OTOA) c2(NP_733764) c3(otoancorin isoform 2) c4(246997) c5(246998, 246999, 247000, 247001, 247002, 247003) c6( ) c7(c, alA, cnm); #c1(OTOA) c2(XP_011544049) c3(otoancorin isoform X1) c4(343222, 343223, 343224) c5(343225, 343226, 343227, 343228, 343229, 343230, 343231, 343232, 343233, 343234, 343235, 343236) c6( ) c7(c, alA, cnm); #c1(OTOA) c2(XP_011544050) c3(otoancorin isoform X2) c4( ) c5(343237, 343238, 343239, 343240, 343241, 343242) c6( ) c7(c, alA, cnm); #c1(OTOF) c2(NP_004793) c3(otoferlin isoform b) c4(149906) c5(149907, 149908, 149909, 149910, 149911) c6( ) c7(bel, mG, alA, cnn, oc, gT, Bh); #c1(OTOF) c2(NP_919224) c3(otoferlin isoform a) c4(258247) c5(258248, 258249, 258250, 258251, 258252, 258253) c6( ) c7(beO, mG, alA, cnn, oc, gT, Bh); #c1(OTOF) c2(NP_919303) c3(otoferlin isoform c) c4(258507) c5(258508, 258509, 258510, 258511, 258512, 258513, 258514) c6( ) c7(beO, mG, alA, cnn, oc, gT, Bh); #c1(OTOF) c2(NP_919304) c3(otoferlin isoform d) c4(258515) c5(258516, 258517, 258518, 258519, 258520, 258521) c6( ) c7(beO, mG, alA, cnn, oc, gT, Bh); #c1(OTOF) c2(XP_005264701) c3(otoferlin isoform X2) c4( ) c5(279214, 279215, 279216, 279217, 279218, 279219) c6( ) c7(bel, mG, alA, cnn, oc, gT, Bh); #c1(OTOF) c2(XP_011531487) c3(otoferlin isoform X1) c4(327617, 327618) c5(327619, 327620, 327621, 327622, 327623, 327624) c6( ) c7(beO, mG, alA, cnn, oc, gT, Bh); #c1(OTOG) c2(NP_001264198) c3(otogelin isoform a precursor) c4(97615) c5(97616, 97617, 97618, 97619, 97620, 97621) c6( ) c7(cno, an, ac, PJ); #c1(OTOG) c2(NP_001278992) c3(otogelin isoform b precursor) c4(111177) c5(111178, 111179, 111180, 111181, 111182, 111183, 111184) c6( ) c7(cno, an, ac, PJ); #c1(OTOGL) c2(NP_775862) c3(otogelin-like protein precursor) c4( ) c5(250505, 250506, 250507, 250508, 250509) c6( ) c7(cnp); #c1(OTOGL) c2(XP_011536493) c3(otogelin-like protein isoform X2) c4(333857) c5(333858) c6( ) c7(cnp); #c1(OTOL1) c2(NP_001073909) c3(otolin-1 precursor) c4(36491) c5(36492, 36493, 36494, 36495, 36496, 36497) c6( ) c7(aur); #c1(OTOP1) c2(NP_819056) c3(otopetrin-1) c4(253147) c5(253148, 253149, 253150, 253151, 253152, 253153, 253154, 253155) c6(253156, 253157) c7(aRa, aQX); #c1(OTOR) c2(NP_064542) c3(otoraplin precursor) c4(202899) c5(202900, 202901, 202902, 202903, 202904, 202905) c6( ) c7(cnq); #c1(OTP) c2(NP_115485) c3(homeobox protein orthopedia) c4(222886) c5(222887, 222888, 222889, 222890, 222891, 222892, 222893) c6( ) c7(Yi); #c1(DTUB1) c2(NP_060140) c3(ubiquitin thioesterase OTUB1) c4(194368, 194369, 194370) c5(194371, 194372, 194373, 194374, 194375, 194376, 194377, 194378, 194379, 194380, 194381, 194382, 194383, 194384, 194385, 194386, 194387, 194388, 194389, 194390, 194391) c6( ) c7(t, hc, uE); #c1(OTUD1) c2(NP_001138845) c3(OTU domain-containing protein 1) c4(57931) c5(57932, 57933, 57934, 57935, 57936, 57937) c6( ) c7(uQ); #c1(OTUD4) c2(NP_001096123) c3(OTU domain-containing protein 4 isoform 3) c4(41071) c5(41072, 41073, 41074, 41075, 41076) c6( ) c7(ck, bv, T, ft, kP, s, eM, bP, aW, ds, Xc, ff, jh, aT, ci, en, g, cm, bt, ad); #c1(OTUD4) c2(NP_059963) c3(OTU domain-containing protein 4 isoform 2) c4( ) c5(193396, 193397, 193398, 193399, 193400, 193401, 193402, 193403, 193404, 193405, 193406, 193407, 193408, 193409) c6( ) c7(ck, bv, T, ft, kP, s, eM, bP, aW, ds, Xc, ff, jh, aT, ci, en, g, cm, bt, ad); #c1(OTUD4) c2(XP_005263136) c3(OTU domain-containing protein 4 isoform X4) c4(278327, 278328, 278329) c5(278330, 278331, 278332, 278333, 278334, 278335, 278336, 278337, 278338) c6( ) c7(ck, bv, T, ft, kP, s, eM, bP, aW, ds, Xc, ff, jh, aT, ci, en, g, cm, bt, ad); #c1(OTUD7A) c2(NP_570971) c3(OTU domain-containing protein 7A) c4(234440, 234441) c5(234442, 234443, 234444, 234445, 234446, 234447, 234448, 234449, 234450, 234451, 234452) c6( ) c7(cd, aNO, T, B); #c1(OTUD7A) c2(XP_011519589) c3(OTU domain-containing protein 7A isoform X1) c4(314255) c5(314256, 314257, 314258, 314259, 314260, 314261, 314262) c6( ) c7(cd, aNO, T, B); #c1(OTUD7B) c2(XP_011508087) c3(OTU domain-containing protein 7B isoform X1) c4(298576) c5( ) c6( ) c7(T, B); #c1(OTUD7B) c2(XP_011508090) c3(OTU domain-containing protein 7B isoform X2) c4( ) c5(298577) c6( ) c7(T, B); #c1(OTULIN) c2(NP_612357) c3(ubiquitin thioesterase otulin) c4(236230) c5(236231, 236232, 236233, 236234, 236235, 236236) c6( ) c7(T); #c1(OTX1) c2(NP_055377) c3(homeobox protein OTX1) c4(180660) c5(180661, 180662, 180663, 180664, 180665, 180666, 180667, 180668, 180669, 180670, 180671) c6( ) c7(jf, aW, ff, ha, aT, en, rz, ad, bN); #c1(OTX2) c2(NP_001257453) c3(homeobox protein OTX2 isoform b) c4(95436, 95437) c5(95438, 95439, 95440, 95441, 95442, 95443, 95444, 95445, 95446, 95447, 95448, 95449, 95450, 95451, 95452, 95453) c6( ) c7(aMU, LX, ko, cnv, Nh, biN, aMS, HC, aT, bK, an, cnu, cnt, jf, avP, cns, bDf, kj, aW, ff, Rj, chn, en, cnr, nC, kn, ac, bP, dB, eD, cnw, aSp, bgd, oQ); #c1(OTX2) c2(NP_001257454) c3(homeobox protein OTX2 isoform a) c4(95454, 95455) c5(95456, 95457, 95458, 95459, 95460, 95461, 95462, 95463, 95464, 95465, 95466, 95467, 95468) c6( ) c7(aMU, LX, ko, cnv, Nh, biN, aMS, HC, aT, bK, an, cnu, cnt, jf, avP, cns, bDf, kj, aW, ff, Rj, chn, en, cnr, nC, kn, ac, bP, dB, eD, cnw, aSp, bgd, oQ); #c1(OVCA2) c2(NP_543012) c3(ovarian cancer-associated gene 2 protein) c4(233244) c5(233245, 233246, 233247, 233248, 233249, 233250) c6( ) c7(jf, dA, m, bz, j, t, kS); #c1(OVOL1) c2(NP_004552) c3 (putative transcription factor Ovo-like 1) c4(147587) c5(147588, 147589, 147590, 147591, 147592, 147593, 147594) c6( ) c7(eE, cm, T); #c1(OVOL2) c2(NP_067043) c3(transcription factor Ovo-like 2 isoform 1) c4(208587) c5(208588, 208589, 208590, 208591, 208592, 208593, 208594) c6( ) c7(T); #c1(QXA1L) c2(NP_005006) c3(mitochondrial inner membrane protein OXAlL) c4(152014, 152015) c5(152016, 152017, 152018, 152019, 152020, 152021, 152022, 152023, 152024, 152025, 152026, 152027) c6( ) c7(bL, bM, T, if, e, ht, n, j, ci, qN, q, eA, x, cF, py, aE, M, aT, kf, nm, dL, K, hu, pk, db, eL, azh, aii, vT, aW, Y, ff, eH, alB, r, nY, yq, en, g, eF, t, f, bz, cm, ik, dR, eM, ig, eD, at, m, k, fD, adg, B, Rf); #c1(OXCT1) c2(NP_000427) c3(succinyl-CoA:3-ketoacid coenzyme A transferase 1, mitochondrial precursor) c4(8855, 8856, 8857, 8858) c5(8859, 8860, 8861, 8862, 8863, 8864, 8865, 8866, 8867, 8868, 8869, 8870, 8871, 8872, 8873, 8874, 8875, 8876, 8877, 8878, 8879, 8880, 8881, 8882, 8883, 8884, 8885, 8886) c6( ) c7(bL, aq, v, eA, bq, B, bA, cnx); #c1(OXER1) c2(NP_683765) c3(oxoeicosanoid receptor 1) c4(242572) c5(242573, 242574, 242575, 242576, 242577, 242578) c6( ) c7(bL, bM, d, aE, Y, pX, ff, h, Ja, v, dX, en, he, Fg, cE, ad); #c1(OXGR1) c2(XP_005254103) c3(2-oxoglutarate receptor 1 isoform X1) c4(272942, 272943, 272944) c5(272945, 272946, 272947, 272948, 272949, 272950, 272951, 272952, 272953, 272954, 272955, 272956, 272957, 272958, 272959, 272960, 272961, 272962, 272963, 272964, 272965, 272966) c6( ) c7(B); #c1(OXR1) c2(NP_001185461) c3(oxidation resistance protein 1 isoform 3) c4(75626) c5(75627, 75628, 75629, 75630, 75631, 75632) c6( ) c7(U, fq, K, c); #c1(OXR1) c2(NP_001185462) c3(oxidation resistance protein 1 isoform 4) c4(75633) c5(75634, 75635, 75636, 75637, 75638, 75639) c6( ) c7(U, fq, K, c); #c1(OXR1) c2(NP_001185463) c3(oxidation resistance protein 1 isoform 5) c4(75640) c5(75641, 75642, 75643, 75644, 75645, 75646) c6( ) c7(U, fq, K, c); #c1(OXR1) c2(NP_001185464) c3(oxidation resistance protein 1 isoform 6) c4(75647) c5(75648, 75649, 75650, 75651, 75652, 75653) c6( ) c7(U, fq, K, c); #c1(OXR1) c2(NP_060472) c3(oxidation resistance protein 1 isoform 1) c4(196105) c5(196106, 196107, 196108, 196109, 196110, 196111, 196112, 196113, 196114, 196115, 196116) c6( ) c7(U, fq, K, c); #c1(OXR1) c2(NP_851999) c3(oxidation resistance protein 1 isoform 2) c4(254776) c5(254777, 254778, 254779, 254780, 254781, 254782, 254783, 254784, 254785, 254786, 254787) c6( ) c7(U, fq, K, c); #c1(OXR1) c2(XP_005251052) c3(oxidation resistance protein 1 isoform X7) c4(270730) c5(270731, 270732, 270733, 270734, 270735, 270736) c6( ) c7(U, fq, K, c); #c1(OXR1) c2(XP_006716658) c3(oxidation resistance protein 1 isoform X5) c4(290422) c5( ) c6( ) c7(U, fq, K, c); #c1(OXSR1) c2(NP_005100) c3(serine/threonine-protein kinase OSR1) c4(152874) c5(152875, 152876, 152877, 152878, 152879, 152880) c6( ) c7(ff, cz, aA, jy, aOM, aF, ey, K, en, er, qa, ad); #c1(OXT) c2(XP_011527540) c3(oxytocin-neurophysin 1 isoform X1) c4(322354) c5(322355, 322356, 322357, 322358, 322359, 322360) c6( ) c7(gh, uk, DV, bO, cz, To, bS, v, ct, ci, zP, ad, aqN, ft, Bc, iX, px, QU, aT, lG, cy, K, lQ, qM, he, yj, ae, dl, rz, nJ, agK, CN, aW, ff, eH, en, Eg, brk, Jl, Z, bz, f, eE, bN, hq, eF, cm, yX, pu, bb, cA, X, cbE, m, ra, ZW, sC, agZ); #c1(OXTR) c2(NP_000907) c3(oxytocin receptor) c4(17083, 17084, 17085) c5(17086, 17087, 17088, 17089, 17090, 17091, 17092, 17093, 17094, 17095, 17096, 17097, 17098, 17099, 17100, 17101, 17102) c6( ) c7(bM, sY, T, uk, alc, bO, cz, bS, j, ct, gf, ci, v, aqN, av, b, ft, iX, pX, aT, adB, K, ahg, he, rz, aqC, dl, VX, aW, eH, aeS, Eg, Jl, Z, uC, bz, eF, op, bN, avK, jq, hq, eM, vA, Uv, m, amu, i, ra, agZ, ckr); #c1(P2RX1) c2(NP_002549) c3(P2X purinoceptor 1) c4(129727) c5(129728, 129729, 129730, 129731, 129732, 129733) c6( ) c7(cA, c, ig, lT, yx, aT, cny, bk, K, iq); #c1(P2RX2) c2(NP_001269093) c3(P2X purinoceptor 2 isoform J) c4(100424) c5(100425, 100426, 100427, 100428, 100429, 100430) c6( ) c7(cA, c, ig, iT, yx, ha, aT, bK, cnz); #c1(P2RX2) c2(NP_036358) c3(P2X purinoceptor 2 isoform 1) c4(173212) c5(173213, 173214, 173215, 173216, 173217, 173218) c6( ) c7(cA, c, ig, iT, yx, ha, aT, bk, cnz); #c1(P2RX2) c2(NP_057402) c3(PZX purinoceptor 2 isoform C) c4(190560) c5(190561, 190562, 190563, 190564, 190565, 190566) c6( ) c7(cA, c, ig, iT, yx, ha, aT, bK, cnz); #c1(P2RX2) c2(NP_733782) c3(P2X purinoceptor 2 isoform A) c4(247075) c5(247076, 247077, 247078, 247079, 247080, 247081) c6( ) c7(cA, c, ig, iT, yx, ha, aT, bK, cnz); #c1(P2RX2) c2(NP_733783) c3(P2X purinoceptor 2 isoform D) c4(247082) c5(247083, 247084, 247085, 247086, 247087, 247088) c6( ) c7(cA, c, ig, iT, yx, ha, aT, bK, cnz); #c1(P2RX2) c2(NP_777361) c3(P2X purinoceptor 2 isoform H) c4(251094) c5(251095, 251096, 251097, 251098, 251099, 251100) c6( ) c7(cA, c, ig, iT, yx, ha, aT, bK, cnz); #c1(P2RX2) c2(NP_777362) c3(P2X purinoceptor 2 isoform B) c4(251101) c5(251102, 251103, 251104, 251105, 251106, 251107) c6( ) c7(cA, c, ig, iT, yx, ha, aT, bK, cnz); #c1(P2RX3) c2(XP_011543372) c3(P2X purinoceptor 3 isoform X1) c4(342217, 342218) c5(342219, 342220, 342221, 342222, 342223, 342224, 342225, 342226, 342227, 342228, 342229, 342230) c6( ) c7(aUh, dP, T, c, cA, xZ, ig, iT, yx, eH, aT, bK, ajC); #c1(P2RX4) c2(NP_001243725) c3(P2X purinoceptor 4 isoform 1) c4(91591) c5(91592, 91593, 91594, 91595, 91596, 91597) c6( ) c7(W, c, cA, ig, iT, yx, ha, aT, cz, bk, il, cd, hq); #c1(P2RX4) c2(NP_001248326) c3(P2X purinoceptor 4 isoform 3) c4(93679, 93680) c5(93681, 93682, 93683, 93684, 93685, 93686, 93687) c6( ) c7(W, c, cA, ig, iT, yx, ha, aT, cz, bk, il, cd, hq); #c1(PZRX4) c2(NP_001248327) c3(P2X purinoceptor 4 isoform 4) c4( ) c5(93688, 93689, 93690, 93691, 93692) c6( ) c7(W, c, cA, ig, iT, yx, ha, aT, cz, bK, il, cd, hq); #c1(PZRX4) c2(NP_002551) c3(P2X purinoceptor 4 isoform 2) c4(129734, 129735) c5(129736, 129737, 129738, 129739, 129740, 129741, 129742, 129743, 129744, 129745, 129746, 129747) c6( ) c7(W, c, cA, ig, iT, yx, ha, aT, cz, bK, il, cd, hq); #c1(P2RX5) c2(NP_001191448) c3(PZX purinoceptor 5 isoform D) c4(81564) c5(81565, 81566, 81567, 81568, 81569, 81570) c6( ) c7(ck, xQ, T, cF, c, cA, ig, aW, hk, yx, qt, iT, iU, aT, fe, bK, im, bt, bpx); #c1(P2RX5) c2(NP_001191449) c3(P2X purinoceptor 5 isoform E) c4(81571, 81572) c5(81573, 81574, 81575, 81576, 81577, 81578, 81579, 81580, 81581, 81582, 81583, 81584) c6( ) c7(ck, xQ, T, cF, c, cA, ig, aW, hk, yx, qt, iT, iU, aT, fe, bK, im, bt, bpx); #c1(P2RX5) c2(NP_002552) c3(P2X purinoceptor 5 isoform A) c4(129748, 129749) c5(129750, 129751, 129752, 129753, 129754, 129755, 129756, 129757, 129758, 129759, 129760, 129761) c6( ) c7(ck, xQ, T, cF, c, cA, ig, aW, hk, yx, qt, iT, iU, aT, fe, bK, im, bt, bpx); #c1(P2RX5) c2(NP_778255) c3(P2X purinoceptor 5 isoform B) c4(251523) c5(251524, 251525, 251526, 251527, 251528, 251529) c6( ) c7(ck, xQ, T, cF, c, cA, ig, aW, hk, yx, qt, iT, iU, aT, fe, bK, im, bt, bpx); #c1(P2RX6) c2(NP_001153026) c3(P2X purinoceptor 6 isoform 2) c4(60335, 60336) c5(60337, 60338, 60339, 60340, 60341, 60342, 60343, 60344, 60345, 60346, 60347, 60348, 60349, 60350, 60351, 60352, 60353, 60354, 60355, 60356, 60357, 60358, 60359, 60360) c6( ) c7(T, QG, ac, c, cA, ig, iT, Or, e, yx, aw, aT, cz, bK, j, eu); #c1(P2RX6) c2(NP_005437) c3(P2X purinoceptor 6 isoform 1) c4(156169, 156170, 156/71) c5(156172, 156173, 156174, 156175, 156176, 156177, 156178, 156179, 156180, 156181, 156182, 156183) c6( ) c7(T, QG, ac, c, cA, ig, iT, Or, e, yx, aw, aT, cz, bK, j, eu); #c1(P2RX6) c2(XP_011528802) c3(PZX purinoceptor 6 isoform X3) c4(324167) c5( ) c6( ) c7(T, QG, ac, c, cA, ig, iT, Or, e, yx, aw, aT, cz, bK, j, eu); #c1(P2RX6) c2(XP_011528804) c3(P2X purinoceptor 6 isoform X6) c4( ) c5(324168) c6( ) c7(T, QG, ac, c, cA, ig, iT, Or, e, yx, aw, aT, cz, bK, j, eu); #c1(P2RX7) c2(NP_002553) c3(P2X purinoceptor 7) c4(129762, 129763, 129764) c5(129765, 129766, 129767, 129768, 129769, 129770, 129771, 129772, 129773, 129774, 129775, 129776, 129777) c6( ) c7(bL, bM, T, bO, cz, fd, mS, aj, bS, gf, ct, j, pz, il, V, cd, J, ad, fU, abk, wv, aE, Db, mW, M, aT, cy, iT, hn, bK, dL, wb, ji, oi, mY, mV, ae, bA, ml, aus, aW, Y, vj, Tr, yx, eZ, zD, ig, Qv, fe, g, hf, Z, cm, jb, bw, mZ, mX, ck, hq, pH, nM, W, c, nB, xZ, hk, anY, hc, cA, Ei, U, cj, vL, at, sv, nw, dy, wB, qF, ei, y, bt, mT, avY); #c1(P2RX7) c2(XP_011536720) c3(P2X purinoceptor 7 isoform X1) c4(334317, 334318) c5(334319, 334320, 334321, 334322, 334323, 334324, 334325, 334326, 334327, 334328, 334329, 334330, 334331, 334332, 334333) c6( ) c7(bL, bM, T, bO, cz, fd, mS, aj, bS, gf, ct, j, pz, il, V, cd, J, ad, fU, abk, w, aE, Db, mW, M, aT, cy, iT, hn, bK, dL, wb, ji, oi, mY, mV, ae, bA, mU, aus, aW, Y, vj, Tr, yx, eZ, zD, ig, Qv, fe, g, hf, Z, cm, jb, bw, mZ, mX, ck, hq, pH, nM, W, c, nB, xZ, hk, anY, hc, cA, Ei, U, cj, vL, at, sv, nw, dy, wB, qF, ei, y, bt, mT, avY); #c1(P2RX7) c2(XP_011536721) c3(P2X purinoceptor 7 isoform X2) c4( ) c5(334334, 334335, 334336, 334337, 334338, 334339) c6( ) c7(bL, bM, T, bO, cz, fd, mS, aj, bS, gf, ct, j, pz, il, V, cd, J, ad, fU, abk, wv, aE, Db, mW, M, aT, cy, iT, hn, bK, dL, wb, ji, oi, mY, mV, ae, bA, mU, aus, aW, Y, vj, Tr, yx, eZ, zD, ig, Av, fe, g, hf, Z, cm, jb, bw, mZ, mX, ck, hq, pH, nM, W, c, nB, xZ, hk, anY, hc, cA, Ei, U, cj, vL, at, sv, nw, dy, wB, qF, ei, y, bt, mT, avY); #c1(P2RX7) c2(XP_011536722) c3(PZX purinoceptor 7 isoform X3) c4(334340) c5(334341, 334342, 334343, 334344, 334345) c6( ) c7(bL, bM, T, bO, cz, fd, mS, aj, bS, gf, ct, j, pz, il, V, cd, J, ad, fU, abk, w, aE, Db, mW, M, aT, cy, iT, hn, bK, dL, wb, ji, oi, mY, mV, ae, bA, mU, aus, aW, Y, vj, Tr, yx, eZ, zD, ig, Qv, fe, g, hf, Z, cm, jb, bw, mZ, mX, ck, hq, pH, nM, W, c, nB, xZ, hk, anY, hc, cA, Ei, U, cj, vL, at, sv, nw, dy, wB, qF, ei, y, bt, mT, avY); #c1(P2RY1) c2(NP_002554) c3(P2Y purinoceptor 1) c4(129778) c5(129779, 129780, 129781, 129782, 129783, 129784) c6( ) c7(dD, j, eq, eA, ad, aT, h, bk, K, ae, yx, eZ, kD, EZ, as, c, cA, ig, hk, iT, cj, sU, dX); #c1(P2RY11) c2(NP_002557) c3(P2Y purinoceptor 11) c4( ) c5(129792, 129793, 129794, 129795, 129796, 129797) c6( ) c7(T, aw, cF, ao, hm); #c1(P2RY12) c2(NP_795345) c3(P2Y purinoceptor (2) c4(252423, 252424) c5(252425, 252426, 252427, 252428, 252429, 252430, 252431, 252432, 252433, 252434, 252435, 252436) c6( ) c7(P, T, dD, eR, e, aw, cz, BH, aj, j, eq, J, fU, M, K, GO, af, hm, fO, eZ, kD, iy, vs, alf, cm, kT, ac, bm, cny, aS, dX, ei, wx); #c1(P2RY13) c2(NP_795713) c3(PZY purinoceptor (3) c4(252462) c5(252463, 252464, 252465, 252466, 252467, 252468) c6( ) c7(ea, dD, eq, iV); #c1(P2RY14) c2(NP_055694) c3(P2Y purinoceptor 14) c4(182811, 182812) c5(182813, 182814, 182815, 182816, 182817, 182818, 182819, 182820, 182821, 182822, 182823, 182824, 182825, 182826, 182827, 182828) c6( ) c7(lV); #c1(P2RY2) c2(NP_788085) c3(P2Y purinoceptor 2) c4( ) c5(252173) c6( ) c7(oO, T, dD, ea, zt, aw, cz, eq, ad, av, aE, aT, h, bK, ae, af, aW, yx, eH, cl, eF, cm, wU, qa, ek, ac, c, cA, ig, hk, iT, U, hL, B, ao); #c1(P2RY2) c2(XP_011543376) c3(P2Y purinoceptor 2 isoform X1) c4(342231) c5(342232, 342233, 342234, 342235, 342236, 342237, 342238, 342239, 342240, 342241, 342242, 342243, 342244, 342245) c6( ) c7(oO, T, dD, ea, zt, aw, cz, eq, ad, av, aE, aT, h, bK, ae, af, aW, yx, eH, cl, eF, cm, wU, qa, ek, ac, c, cA, ig, hk, iT, U, hL, B, ao); #c1(P2RY4) c2(NP_002556) c3(P2Y purinoceptor 4) c4(129785) c5(129786, 129787, 129788, 129789, 129790, 129791) c6( ) c7(cm); #c1(P2RY6) c2(NP_001264134) c3(P2Y purinoceptor 6 isoform 1) c4(97543) c5(97544, 97545, 97546, 97547, 97548, 97549, 97550, 97551, 97552, 97553, 97554, 97555, 97556, 97557, 97558, 97559, 97560, 97561, 97562, 97563, 97564, 97565, 97566, 97567, 97568, 97569, 97570, 97571, 97572, 97573, 97574, 97575, 97576, 97577, 97578, 97579) c6( ) c7(av, aE, dh, tv, tu, as, cm); #c1(P2RY6) c2(NP_001264137) c3(P2Y purinoceptor 6 isoform 2) c4( ) c5(97580, 97581) c6( ) c7(av, aE, dh, tv, tu, as, cm); #c1(P2RY6) c2(XP_011543378) c3(P2Y purinoceptor 6 isoform X3) c4(342246, 342247) c5(342248, 342249, 342250, 342251, 342252, 342253, 342254, 342255) c6( ) c7(av, aE, dh, tv, tu, as, cm); #c1(PZRY8) c2(XP_005274835) c3(P2Y purinoceptor 8 isoform X2) c4(285485) c5(285486, 285487, 285488, 285489, 285490) c6( ) c7(ck, x, k, nA, ajo, f, hF); #c1(P2RY8) c2(XP_006724927) c3(P2Y purinoceptor 8 isoform X1) c4( ) c5(296493) c6( ) c7(ck, x, k, nA, ajo, f, hF); #c1(P3H2) c2(NP_001127890) c3(prolyl 3-hydroxylase 2 isoform b) c4(49432) c5( ) c6( ) c7(T, dB, aW, Y, aT, cnA, af); #c1(P3H2) c2(NP_060662) c3(prolyl 3-hydroxylase 2 isoform a precursor) c4(197035) c5(197036, 197037, 197038, 197039, 197040, 197041, 197042, 197043, 197044, 197045, 197046, 197047, 197048, 197049, 197050, 197051) c6( ) c7(T, dB, aW, Y, aT, cnA, af); #c1(P3H3) c2(NP_055077) c3(prolyl 3-hydroxylase 3 precursor) c4(178793, 178794, 178795) c5(178796, 178797, 178798, 178799, 178800, 178801, 178802, 178803, 178804, 178805, 178806, 178807, 178808, 178809, 178810, 178811, 178812, 178813, 178814, 178815, 178816) c6( ) c7(AK, ai, T, d, c, sO, ds, aWL, aWM, aW, asD, aT, Pg, Ol, Vd, ci, bU, bwg, af); #c1(P3H4) c2(NP_006446) c3(synaptonemal complex protein SC65 precursor) c4(165293, 165294) c5(165295, 165296, 165297, 165298, 165299, 165300, 165301, 165302, 165303, 165304, 165305, 165306, 165307, 165308) c6( ) c7(lN, ll); #c1(P3H4) c2(XP_006721703) c3(synaptonemal complex protein SC65 isoform X1) c4( ) c5(294243, 294244) c6( ) c7(lN, ll); #c1(P4HA1) c2(NP_000908) c3(prolyl 4-hydroxylase subunit alpha-isoform 1 precursor) c4(17103) c5(17104, 17105, 17106, 17107, 17108, 17109, 17110, 17111, 17112, 17113) c6( ) c7(aW, aT); #c1(P4HA1) c2(NP_001136067) c3(prolyl 4-hydroxylase subunit alpha-1 isoform 2 precursor) c4(54231) c5(54232, 54233, 54234, 54235, 54236, 54237, 54238, 54239, 54240, 54241, 54242, 54243, 54244, 54245) c6( ) c7(aW, aT); #c1(P4HA1) c2(NP_001136068) c3 (prolyl 4-hydroxylase subunit alpha-isoform 3 precursor) c4(54246) c5(54247, 54248, 54249, 54250, 54251, 54252) c6( ) c7(aW, aT); #c1(P4HA2) c2(XP_005272177) c3 (prolyl 4-hydroxylase subunit alpha-2 isoform X1) c4(283733, 283734) c5(283735, 283736, 283737, 283738, 283739, 283740, 283741, 283742, 283743, 283744, 283745, 283746, 283747, 283748, 283749, 283750) c6( ) c7(Dw, dA, aW, oe, aT, cm, Ds, t); #c1(P4HA2) c2(XP_006714793) c3 (prolyl 4-hydroxylase subunit alpha-2 isoform X2) c4(288160) c5(288161, 288162, 288163, 288164, 288165, 288166, 288167, 288168, 288169, 288170, 288171, 288172, 288173, 288174) c6( ) c7(Dw, dA, aW, oe, aT, cm, Ds, t); #c1(P4HA2) c2(XP_011542007) c3(prolyl 4-hydroxylase subunit alpha-2 isoform X3) c4(340505, 340506, 340507, 340508, 340509, 340510, 340511) c5(340512, 340513, 340514, 340515, 340516, 340517, 340518, 340519, 340520, 340521, 340522, 340523, 340524, 340525, 340526, 340527, 340528, 340529, 340530, 340531, 340532, 340533, 340534, 340535, 340536, 340537, 340538, 340539, 340540, 340541, 340542, 340543, 340544, 340545, 340546, 340547, 340548, 340549, 340550, 340551, 340552, 340553, 340554, 340555, 340556, 340557, 340558, 340559) c6( ) c7(Dw, dA, aW, oe, aT, cm, Ds, t); #c1(P4HB) c2(NP_000909) c3(protein disulfide-isomerase precursor) c4(17114, 17115, 17116) c5(17117, 17118, 17119, 17120, 17121, 17122, 17123, 17124, 17125, 17126, 17127, 17128, 17129, 17130, 17131, 17132, 17133, 17134) c6( ) c7(bL, T, dt, aw, aA, aj, j, P, J, cd, amW, J, ad, eA, akN, F, xE, dV, aT, h, cy, ZU, K, eu, bA, ul, A, qt, aW, lo, aYR, aF, cm, ac, c, kg, dh, AH, ll, U, y, bt, B); #c1(P4HTM) c2(NP_808807) c3(transmembrane prolyl 4-hydroxylase isoform c) c4(252937) c5(252938, 252939, 252940, 252941, 252942, 252943) c6( ) c7(eF, beH, beJ, eH); #c1(P4HTM) c2(NP_808808) c3(transmembrane prolyl 4-hydroxylase isoform a) c4(252944) c5(252945, 252946, 252947, 252948, 252949, 252950) c6( ) c7(eF, beH, beJ, eH); #c1(PAZG4) c2(NP_006182) c3(proliferation-associated protein 2G4) c4(162657, 162658) c5(162659, 162660, 162661, 162662, 162663, 162664, 162665, 162666, 162667, 162668, 162669, 162670) c6( ) c7(T, dB, aW, qK, aT, aA, ci, g, ae, LS, ad); #c1(PABPC1) c2(XP_005250918) c3(polyadenylate-binding protein 1 isoform X1) c4(270641, 270642, 270643, 270644, 270645, 270646, 270647, 270648, 270649, 270650, 270651) c5(270652, 270653, 270654, 270655, 270656, 270657, 270658, 270659, 270660, 270661, 270662, 270663, 270664, 270665, 270666, 270667, 270668, 270669, 270670, 270671, 270672, 270673, 270674, 270675, 270676, 270677, 270678, 270679, 270680, 270681, 270682, 270683, 270684, 270685, 270686, 270687, 270688, 270689, 270690, 270691, 270692, 270693, 270694, 270695, 270696, 270697, 270698, 270699, 270700, 270701, 270702, 270703, 270704, 270705, 270706, 270707, 270708, 270709, 270710, 270711, 270712, 270713, 270714, 270715, 270716) c6( ) c7(ae, aoU, aW, Ol, Dq, ht, aT, h, lo, app, K, bz, ad); #c1(PABPC3) c2(NP_112241) c3(polyadenylate-binding protein 3) c4(221072) c5(221073, 221074, 221075, 221076, 221077, 221078, 221079) c6( ) c7(fq); #c1(PABPC4L) c2(NP_00108206) c3(polyadenylate-binding protein 4-like) c4(43140, 43141, 43142) c5(43143, 43144, 43145, 43146, 43147, 43148, 43149, 43150, 43151, 43152, 43153) c6( ) c7(j, pN); #c1(PABPN1) c2(NP_004634) c3(polyadenylate-binding protein 2) c4(148389, 148390) c5(148391, 148392, 148393, 148394, 148395, 148396, 148397, 148398, 148399, 148400, 148401, 148402) c6( ) c7(no, af, eM, Ae, aku, bAj, ds, xs, cnB, Ak, oj, app, zW, bZP, dr); #c1(PACRG) c2(NP_001073848) c3(parkin coregulated gene protein isoform 2) c4(36116) c5(36117, 36118, 36119, 36120, 36121, 36122, 36123, 36124, 36125, 36126) c6( ) c7(ck, x, AW, gU, py, iU, pN, aWe, ahD, vm, k, an, aMB); #c1(PACRG) c2(NP_689623) c3(parkin coregulated gene protein isoform 1) c4(243324) c5(243325, 243326, 243327, 243328, 243329, 243330) c6( ) c7(ck, x, AW, gU, py, iU, pN, aWe, ahD, vm, k, an, aMB); #c1(PACS1) c2(NP_060496) c3 (phosphofurin acidic cluster sorting protein 1) c4(196239, 196240) c5(196241, 196242, 196243, 196244, 196245, 196246) c6( ) c7(cnC, h, v, qM, nA, lB); #c1(PACS1) c2(XP_011543465) c3(phosphofurin acidic cluster sorting protein 1 isoform X2) c4(342357) c5( ) c6( ) c7(cnC, h, v, qM, nA, lB); #c1(PACS1) c2(XP_011543466) c3 (phosphofurin acidic cluster sorting protein 1 isoform X3) c4( ) c5(342358, 342359, 342360, 342361, 342362, 342363, 342364, 342365, 342366, 342367, 342368, 342369) c6( ) c7(cnC, h, v, qM, nA, lB); #c1(PACS2) c2(NP_001094383) c3(phosphofurin acidic cluster sorting protein 2 isoform 1) c4(40698) c5(40699, 40700, 40701, 40702, 40703, 40704) c6( ) c7(ac); #c1(PACS2) c2(NP_056012) c3(phosphofurin acidic cluster sorting protein 2 isoform 2) c4(185126) c5(185127, 185128, 185129, 185130, 185131, 185132) c6( ) c7(ac); #c1(PACS2) c2(XP_006720154) c3(phosphofurin acidic cluster sorting protein 2 isoform X9) c4(293301) c5(293302, 293303, 293304, 293305, 293306, 293307) c6( ) c7(ac); #c1(PACS1N1) c2(XP_011512843) c3(protein kinase C and casein kinase substrate in neurons protein 1 isoform X1) c4(303903, 303904) c5(303905, 303906, 303907, 303908, 303909, 303910, 303911, 303912, 303913, 303914, 303915, 303916, 303917, 303918, 303919, 303920) c6( ) c7(rR, ff, ht, ex, eV, en, on, Hr); #c1(PACSlN2) c2(NP_001171899) c3(protein kinase C and casein kinase substrate in neurons protein 2 isoform A) c4(71991, 71992) c5(71993, 71994, 71995, 71996, 71997, 71998, 71999, 72000, 72001, 72002, 72003, 72004, 72005, 72006) c6( ) c7(ck, ad); #c1(PACSIN2) c2(XP_005261376) c3(protein kinase C and casein kinase substrate in neurons protein 2 isoform X1) c4(277317, 277318) c5(277319, 277320, 277321, 277322, 277323, 277324, 277325, 277326, 277327, 277328, 277329, 277330, 277331, 277332, 277333, 277334, 277335, 277336, 277337, 277338, 277339, 277340, 277341, 277342, 277343, 277344, 277345) c6( ) c7(ck, ad); #c1(PACSIN2) c2(XP_011528153) c3(protein kinase C and casein kinase substrate in neurons protein 2 isoform X2) c4(323270) c5(323271, 323272, 323273, 323274, 323275) c6( ) c7(ck, ad); #c1(PADI4) c2(XP_011539452) c3(protein-arginine deiminase type-4 isoform X1) c4(337628, 337629) c5( ) c6( ) c7(T, Qs, rV, pf, e, bB, aj, j, ci, zP, V, Uy, J, ai, yM, ds, afQ, BJ, auB, M, aT, cy, K, bby, hu, A, zQ, bf, vT, aW, Y, ff, Lh, ge, fe, hp, en, g, pL, t, ir, cm, iV, ck, kT, bE, hk, nF, U, Le, KY, y); #c1(PADI4) c2(XP_011539458) c3(protein-arginine deiminase type-4 isoform X6) c4( ) c5(337630, 337631, 337632, 337633, 337634, 337635, 337636, 337637, 337638, 337639, 337640, 337641) c6( ) c7(T, Qs, rV, pf, e, bB, aj, j, ci, zP, V, Uy, J, ai, yM, ds, afQ, BJ, auB, M, aT, cy, K, bby, hu, A, zQ, bf, vT, aW, Y, ff, Lh, ge, fe, hp, en, g, pL, t, ir, cm, iV, ck, KT, bE, hk, nF, U, Le, KY, y); #c1(PAEP) c2(NP_001018059) c3(glycodelin precursor) c4(26979, 26980, 26981, 26982, 26983) c5(26984, 26985, 26986, 26987, 26988, 26989, 26990, 26991, 26992, 26993, 26994, 26995, 26996, 26997, 26998, 26999, 27000, 27001, 27002, 27003, 27004, 27005, 27006, 27007, 27008, 27009) c6( ) c7(T, aT, bB, gf, ci, aE, Co, K, cp, aW, fc, hl, g, cm, bwl, y, AM, vA, cj, m, Cx, atx, xN, n, aj, ct, qN, eW, aZv, h, Df, A, mR, aiB, aq, aGd, pm, op, dB, hL, pg, B, dA, jx, aA, cz, q, J, eA, aT, ae, kl, oV, Y, aeS, r, lo, cl, t, ho, blC, aDv, ac, c, im, gT, vi, gx, Kd, uG, arh, cnD, jh, bS, jE, iJ, ad, hX, dP, F, oW, nJ, d, qt, dN, kD, cnE, hv, hp, af, Z, bz, gk, pc, bE, CO, rH, cP, hE, EB, Cp); #c1(PAEP) c2(XP_011517049) c3(glycodelin isoform X2) c4(310522) c5( ) c6( ) c7(T, aT, bB, gf, ci, aE, Co, K, cp, aW, fc, hl, g, cm, bwQ, y, AM, vA, cj, m, Cx, atx, xN, n, aj, ct, qN, eW, aZv, h, Df, A, mR, aiB, aq, aGd, pm, op, dB, hL, pg, B, dA, jx, aA, cz, q, J, eA, aT, ae, kl, oV, Y, aeS, r, lo, cl, t, ho, blC, aDv, ac, c, im, gT, vi, gx, Kd, uG, arh, cnD, jh, bS, jE, iJ, ad, hX, dP, F, oW, nJ, d, qt, dN, kD, cnE, hv, hp, af, Z, bz, gk, pc, bE, CO, rH, cP, hE, EB, Cp); #c1(PAEP) c2(XP_011517050) c3(glycodelin isoform X3) c4(310523, 310524) c5(310525, 310526, 310527, 310528, 310529, 310530, 310531, 310532, 310533, 310534, 310535, 310536) c6( ) c7(T, aT, bB, gf, ci, aE, Co, K, cp, aW, fc, hl, g, cm, bwQ, y, AM, vA, cj, m, Cx, atx, xN, n, aj, ct, qN, eW, aZv, h, Df, A, mR, aiB, aq, aGd, pm, op, dB, hL, pg, B, dA, jx, aA, cz, q, J, eA, aT, ae, kl, oV, Y, aeS, r, lo, cl, t, ho, blC, aDv, ac, c, im, gT, vi, gx, Kd, uG, arh, cnD, jh, bS, jE, iJ, ad, hX, dP, F, oW, nJ, d, qt, dN, kD, cnE, hv, hp, aF, Z, bz, gk, pc, bE, CO, rH, cP, hE, EB, Cp); #c1(PAEP) c2(XP_011517054) c3(glycodelin isoform X6) c4( ) c5(310537, 310538, 310539, 310540, 310541) c6( ) c7(T, aT, bB, gf, ci, aE, Co, K, cp, aW, fc, hl, g, cm, bwQ, y, AM, vA, cj, m, Cx, atx, xN, n, aj, ct, qN, eW, aZv, h, Df, A, mR, aiB, aq, aGd, pm, op, dB, hL, pg, B, dA, jx, aA, cz, q, J, eA, aT, ae, kl, oV, Y, aeS, r, lo, cl, t, ho, blC, aDv, ac, c, im, gT, vi, gx, Kd, uG, arh, cnD, jh, bS, jE, iJ, ad, hX, dP, F, oW, nJ, d, qt, dN, kD, cnE, hv, hp, aF, Z, bz, gk, pc, bE, CO, rH, cP, hE, EB, Cp); #c1(PAF1) c2(NP_001243755) c3(RNA polymerase II-associated factor 1 homolog isoform 2) c4(91654, 91655) c5(91656, 91657, 91658, 91659, 91660, 91661, 91662, 91663, 91664, 91665, 91666, 91667, 91668, 91669, 91670, 91671, 91672, 91673) c6( ) c7(T, aE, ajx, mE, aq, mL, B); #c1(PAF1) c2(NP_061961) c3(RNA polymerase II-associated factor 1 homolog isoform 1) c4(201325, 201326) c5(201327, 201328, 201329, 201330, 201331, 201332) c6( ) c7(T, aE, ajx, mE, aq, mL, B); #c1(PAFAH1B1) c2(NP_000421) c3(platelet-activating factor acetylhydrolase lB subunit alpha) c4(8789, 8790, 8791) c5(8792, 8793, 8794, 8795, 8796, 8797, 8798, 8799, 8800, 8801, 8802, 8803, 8804, 8805, 8806, 8807, 8808, 8809) c6( ) c7(bM, T, fR, Js, e, qx, fd, aeu, afz, Tz, aR, x, oB, aU, oP, pr, aW, FD, bw, uC, aiv, yd, WO, eM, iT, cj, uo, dW, k, dD, sQ, qy, n, oY, nA, abd, br, cc, h, By, cC, Nr, aix, po, pm, QK, UY, kz, FB, pg, ut, Jo, fZ, ol, j, aT, dz, j, af, jf, iU, cnF, Y, bN, ck, afZ, bdU, bt, sY, aZ, ha, jh, eq, hF, oW, vw, lQ, bdS, pq, hG, et, gK, Mz, gl, kD, bz, ek, Nq, EW, pc, lf, iu); #c1(PAFAH1B2) c2(NP_001171675) c3(platelet-activating factor acetylhydrolase lB subunit beta isoform b) c4(71275, 71276) c5(71277, 71278, 71279, 71280, 71281, 71282) c6( ) c7(ame, fR); #c1(PAFAH1B2) c2(NP_001171676) c3 (platelet-activating factor acetylhydrolase lB subunit beta isoform c) c4(71283) c5(71284, 71285, 71286, 71287, 71288, 71289) c6( ) c7(ame, fR); #c1(PAFAH1B2) c2(NP_001171677) c3(platelet-activating factor acetylhydrolase lB subunit beta isoform d) c4( ) c5(71290, 71291, 71292, 71293, 71294, 71295) c6( ) c7(ame, fR); #c1(PAFAH1B2) c2(NP_002563) c3(platelet-activating factor acetylhydrolase lB subunit beta isoform a) c4(129819) c5(129820, 129821, 129822, 129823, 129824, 129825) c6( ) c7(ame, fR); #c1(PAFAH2) c2(XP_011539830) c3(platelet-activating factor acetylhydrolase 2, cytoplasmic isoform X2) c4(338067, 338068, 338069) c5(338070, 338071, 338072, 338073) c6( ) c7(j); #c1(PAG1) c2(XP_011515867) c3(phosphoprotein associated with glycosphingolipid-enriched microdomains 1 isoform X1) c4(308840) c5(308841, 308842, 308843, 308844, 308845, 308846) c6( ) c7(T, ha, al, jh, it, bB, oY, fd, Kh, ci, C, nA, il, hF, AO, q, ad, aCi, eA, av, aT, ask, ae, pi, avP, je, kS, aW, az, t, bz, all, ck, Qi, gk, nM, iM, eM, kB, bE, CZ, m, k, bt, xg, gD, L); #c1(PAGE1) c2(NP_003776) c3(P antigen family member 1) c4(140741) c5(140742, 140743, 140744, 140745, 140746, 140747) c6( ) c7(T, vT, Y, bX, ae, ad); #c1(PAGE4) c2(NP_008934) c3(P antigen family member 4) c4(169640) c5(169641, 169642, 169643, 169644, 169645, 169646, 169647, 169648, 169649, 169650, 169651, 169652, 169653) c6( ) c7(T, vT, Y, r, aA, n, ae, q, ad); #c1(PAGES) c2(NP_001013453) c3(P antigen family member 5 isoform 2) c4( ) c5(25600, 25601, 25602, 25603, 25604, 25605, 25606, 25607, 25608, 25609) c6( ) c7(T, Y, bu); #c1(PAGES) c2(NP_569734) c3(P antigen family member 5 isoform 1) c4(233907, 233908, 233909) c5(233910, 233911, 233912, 233913, 233914, 233915, 233916, 233917) c6( ) c7(T, Y, bu); #c1(PAGR1) c2(NP_078792) c3(PAXIP1-associated glutamate-rich protein 1) c4(215484) c5(215485, 215486, 215487, 215488, 215489, 215490) c6( ) c7(Bl, W, aW, qr, afs, aT, cj, Zn, bzm, aA, aF, K, m, C, h, t, bjQ, vO, azy, bH, bA); #c1(PAH) c2(NP_000268) c3(phenylalanine-4-hydroxylase) c4(5947, 5948) c5(5949, 5950, 5951, 5952, 5953, 5954, 5955, 5956, 5957, 5958, 5959, 5960, 5961, 5962, 5963, 5964, 5965, 5966, 5967, 5968, 5969, 5970, 5971, 5972, 5973, 5974, 5975, 5976, 5977, 5978, 5979, 5980, 5981, 5982, 5983, 5984, 5985, 5986, 5987, 5988, 5989, 5990, 5991, 5992, 5993, 5994, 5995, 5996, 5997, 5998, 5999, 6000, 6001, 6002, 6003, 6004, 6005, 6006, 6007, 6008, 6009, 6010, 6011, 6012, 6013, 6014, 6015, 6016, 6017, 6018, 6019, 6020, 6021, 6022, 6023, 6024, 6025, 6026, 6027, 6028, 6029, 6030, 6031, 6032, 6033, 6034, 6035, 6036, 6037, 6038, 6039, 6040, 6041, 6042, 6043, 6044) c6(6045, 6046, 6047) c7(lD, bM, T, hl, AL, ht, bB, acU, qj, nA, pM, cnH, ad, BY, HF, b, aji, aT, Ps, jy, dL, K, hu, he, eL, rL, ao, ly, aW, oV, zS, dq, aq, HL, zZ, bz, dr, lx, bN, mO, cnl, eM, Kv, bE, pP, cnG, ei, i, it, B, jr, pC); #c1(PAH) c2(XP_011536724) c3(phenylalanine-4-hydroxylase isoform X1) c4(334346) c5(334347, 334348, 334349, 334350, 334351, 334352, 334353, 334354, 334355, 334356, 334357, 334358, 334359, 334360, 334361, 334362, 334363, 334364) c6(334365, 334366, 334367, 334368) c7(lD, bM, T, hl, AL, ht, bB, acU, qj, nA, pM, cnH, ad, BY, HF, b, aji, aT, Ps, jy, dL, K, hu, he, eL, rL, ao, ly, aW, oV, zS, dq, aq, HL, zZ, bz, dr, lx, bN, mO, cnl, eM, Kv, bE, pP, cnG, ei, i, it, B, jr, pC); #c1(PAICS) c2(NP_001072992) c3(multifunctional protein ADE2 isoform 2) c4(35516) c5(35517, 35518, 35519, 35520, 35521, 35522, 35523, 35524, 35525, 35526) c6( ) c7(adW, T, K, ad, PD); #c1(PAICS) c2(NP_001072993) c3(multifunctional protein ADE2 isoform 1) c4(35527) c5(35528, 35529, 35530, 35531, 35532, 35533) c6( ) c7(adW, T, K, ad, PD); #c1(PA1P1) c2(NP_006442) c3(polyadenylate-binding protein-interacting protein 1 isoform 1) c4(165258, 165259) c5(165260, 165261, 165262, 165263, 165264, 165265, 165266) c6( ) c7(j); #c1(PA1P1) c2(NP_877590) c3(polyadenylate-binding protein-interacting protein 1 isoform 2) c4(256917, 256918, 256919) c5(256920, 256921, 256922, 256923, 256924, 256925, 256926, 256927, 256928, 256929, 256930, 256931, 256932, 256933) c6( ) c7(j); #c1(PA1P1) c2(NP_899152) c3(polyadenylate-binding protein-interacting protein 1 isoform 3) c4( ) c5(257961, 257962, 257963, 257964, 257965, 257966, 257967, 257968, 257969, 257970, 257971, 257972, 257973, 257974, 257975, 257976, 257977, 257978, 257979, 257980) c6( ) c7(j); #c1(PA1P1) c2(XP_011512240) c3(polyadenylate-binding protein-interacting protein 1 isoform X2) c4(302814, 302815, 302816) c5(302817) c6( ) c7(j); #c1(PAIP2) c2(XP_011541730) c3(polyadenylate-binding protein-interacting protein 2 isoform X1) c4(340315, 340316) c5(340317, 340318, 340319, 340320, 340321, 340322, 340323, 340324, 340325, 340326, 340327, 340328, 340329, 340330, 340331, 340332, 340333, 340334, 340335, 340336) c6( ) c7(bw, lo, ci, K); #c1(PAK1) c2(NP_002567) c3(serine/threonine-protein kinase PAK 1 isoform 2) c4(129826, 129827, 129828) c5(129829, 129830, 129831, 129832, 129833, 129834, 129835, 129836, 129837, 129838, 129839, 129840, 129841, 129842, 129843, 129844, 129845, 129846, 129847, 129848, 129849, 129850, 129851, 129852, 129853, 129854, 129855, 129856, 129857, 129858, 129859, 129860, 129861, 129862, 129863, 129864, 129865) c6( ) c7(T, afa, dA, fR, yk, jh, aA, bB, n, fr, ci, nA, q, ad, hX, dP, bv, hd, aoc, Ot, aT, h, cy, By, hP, K, an, wt, nJ, ae, af, eu, WZ, d, ZH, s, CN, aW, Y, ff, r, aq, aF, en, g, t, f, bz, es, KT, cT, Ew, eM, nB, bE, iA, dO, U, aSp, mf, m, bnl, B, y); #c1(PAK1) c2(XP_011543386) c3(serine/threonine-protein kinase PAK 1 isoform X1) c4(342256, 342257, 342258, 342259, 342260) c5(342261, 342262, 342263, 342264, 342265, 342266, 342267, 342268, 342269, 342270, 342271) c6( ) c7(T, afa, dA, fR, yk, jh, aA, bB, n, fr, ci, nA, q, ad, hX, dP, bv, hd, aoc, Ot, aT, h, cy, By, hP, K, an, wt, nJ, ae, af, eu, WZ, d, ZH, s, CN, aW, Y, ff, r, aq, aF, en, g, t, f, bz, es, kT, cT, Ew, eM, nB, bE, iA, do, U, aSp, mf, m, bnl, B, y); #c1(PAK1IP1) c2(NP_060376) c3(p21-activated protein kinase-interacting protein 1) c4(195688) c5(195689, 195690, 195691, 195692, 195693, 195694) c6( ) c7(K); #c1(PAK2) c2(NP_002568) c3(serine/threonine-protein kinase PAK 2) c4(129866) c5(129867, 129868, 129869, 129870, 129871, 129872) c6( ) c7(iQ, ZH, CN, aoc, qt, aW, hl, aT, aA, h, lo, K, m, wt, t, Pr, af, ad); #c1(PAK3) c2(NP_001121639) c3(serine/threonine-protein kinase PAK 3 isoform a) c4(45954) c5(45955, 45956, 45957, 45958, 45959, 45960, 45961, 45962, 45963, 45964, 45965, 45966, 45967, 45968) c6( ) c7(T, dD, fM, ea, aw, Kk, aoE, ha, aA, eq, nA, q, ad, cnJ, x, pX, nd, aT, h, By, dL, bl, K, jj, ae, Lu, je, kS, Jl, aW, Y, ff, eZ, r, nf, gl, Ej, en, cl, cm, bz, op, Hp, LB, iM, bP, eM, afZ, vA, U, cj, at, k, xg); #c1(PAK3) c2(NP_001121640) c3(serine/threonine-protein kinase PAK 3 isoform b) c4(45969, 45970) c5(45971, 45972, 45973, 45974, 45975, 45976, 45977, 45978, 45979, 45980, 45981, 45982, 45983, 45984, 45985, 45986) c6( ) c7(T, dD, fM, ea, aw, Kk, aoE, ha, aA, eq, nA, q, ad, cnJ, x, pX, nd, aT, h, By, dL, bl, K, jj, ae, Lu, je, kS, Jl, aW, Y, ff, eZ, r, nf, gl, Ej, en, cl, cm, bz, op, Hp, LB, iM, bP, eM, afZ, vA, U, cj, at, k, xg); #c1 (PAK3) c2(NP_001121644) c3(serine/threonine-protein kinase PAK 3 isoform c) c4(45987) c5(45988, 45989, 45990, 45991, 45992, 45993, 45994, 45995, 45996, 45997) c6( ) c7(T, dD, fM, ea, aw, Kk, aoE, ha, aA, eq, nA, q, ad, cnJ, x, pX, nd, aT, h, By, dL, bl, K, jj, ae, Lu, je, kS, Jl, aW, Y, ff, eZ, r, nf, gl, Ej, en, cl, cm, bz, op, Hp, LB, iM, bP, eM, afZ, vA, U, cj, at, k, xg); #c1(PAK3) c2(NP_001121645) c3(serine/threonine-protein kinase PAK 3 isoform d) c4(45998) c5(45999, 46000, 46001, 46002, 46003, 46004, 46005, 46006, 46007, 46008) c6( ) c7(T, dD, fM, ea, aw, Kk, aoE, ha, aA, eq, nA, q, ad, cnJ, x, pX, nd, aT, h, By, dL, bl, K, jj, ae, Lu, je, KS, Jl, aW, Y, ff, eZ, r, nf, gl, Ej, en, cl, cm, bz, op, Hp, LB, iM, bP, eM, afZ, vA, U, cj, at, k, xg); #c1(PAK4) c2(NP_001014832) c3(serine/threonine-protein kinase PAK 4 isoform 1) c4(26007, 26008, 26009) c5(26010, 26011, 26012, 26013, 26014, 26015, 26016, 26017, 26018, 26019, 26020, 26021, 26022, 26023, 26024, 26025, 26026, 26027, 26028, 26029, 26030, 26031, 26032, 26033) c6( ) c7(T, aC, ZH, CN, aoc, bE, ff, rA, aA, bB, aF, ci, K, an, en, eA, nJ, ae, B, ad); #c1(PAK4) c2(NP_001014834) c3(serine/threonine-protein kinase PAK 4 isoform 2) c4(26034) c5(26035, 26036, 26037, 26038, 26039, 26040, 26041, 26042, 26043, 26044, 26045, 26046, 26047, 26048, 26049, 26050) c6( ) c7(T, aC, ZH, CN, aoc, bE, ff, rA, aA, bB, aF, ci, K, an, en, eA, nJ, ae, B, ad); #c1(PAK6) c2(NP_001263646) c3(serine/threonine-protein kinase PAK 6 isoform 1) c4(97323) c5(97324, 97325, 97326, 97327, 97328, 97329, 97330, 97331, 97332, 97333, 97334, 97335, 97336, 97337, 97338, 97339, 97340, 97341) c6( ) c7(T, aW, aT, at, dL, K, ae, ad); #c1(PAK6) c2(NP_001263647) c3(serine/threonine-protein kinase PAK 6 isoform 2) c4(97342, 97343, 97344, 97345, 97346, 97347) c5(97348, 97349, 97350, 97351, 97352, 97353, 97354, 97355, 97356, 97357, 97358, 97359, 97360, 97361, 97362, 97363, 97364, 97365, 97366, 97367, 97368, 97369, 97370, 97371, 97372, 97373, 97374, 97375, 97376, 97377, 97378, 97379, 97380, 97381, 97382, 97383) c6( ) c7(T, aW, aT, at, dL, K, ae, ad); #c1(PAK7) c2(NP_065074) c3(serine/threonine-protein kinase PAK 7) c4(203647, 203648) c5(203649, 203650, 203651, 203652, 203653, 203654, 203655, 203656, 203657, 203658, 203659, 203660, 203661, 203662, 203663, 203664, 203665, 203666, 203667, 203668) c6( ) c7(T, W, aW, aT, aA, gl, aF, fu, an, dw, q); #c1(PALB2) c2(NP_078951) c3(partner and localizer of BRCA2) c4(216235) c5(216236, 216237, 216238, 216239, 216240, 216241) c6( ) c7(T, os, aC, cz, aj, oZ, j, ad, er, aE, oW, aT, qv, bK, adV, ae, qw, Ub, aW, Y, g, zE, t, ajj, es, kP, cnl, afd, cnK, m, MC, y, aed); #c1(PALD1) c2(NP_055246) c3(paladin) c4(180018, 180019, 180020) c5(180021, 180022, 180023, 180024, 180025, 180026, 180027, 180028, 180029, 180030, 180031, 180032, 180033, 180034, 180035, 180036, 180037, 180038) c6( ) c7(T, dD, aC, eJ, aE, bE, aW, ea, oe, aT, bB, v, eq, kp, g, X); #c1(PALLD) c2(NP_001159580) c3(palladin isoform 1) c4(64340) c5(64341, 64342, 64343, 64344, 64345) c6( ) c7(T, dD, aC, ea, e, aw, bB, j, eq, eJ, aE, pN, aT, K, cnM, eu, aW, kp, g, ac, bE, pw, ei, X); #c1(PALLD) c2(NP_001159581) c3(palladin isoform 3) c4(64346) c5( ) c6( ) c7(T, dD, aC, ea, e, aw, bB, j, eq, eJ, aE, pN, aT, K, cnM, eu, aW, kp, g, ac, bE, pw, ei, X); #c1(PALLD) c2(NP_001159582) c3(palladin isoform 4) c4(64347) c5(64348, 64349, 64350, 64351, 64352, 64353, 64354, 64355, 64356, 64357, 64358, 64359) c6( ) c7(T, dD, aC, ea, e, aw, bB, j, eq, eJ, aE, pN, aT, K, cnM, eu, aW, kp, g, ac, bE, pw, ei, X); #c1(PALLD) c2(NP_057165) c3(palladin isoform 2) c4(189261, 189262) c5(189263, 189264, 189265, 189266, 189267, 189268, 189269, 189270, 189271, 189272) c6( ) c7(T, dD, aC, ea, e, aw, bB, j, eq, eJ, aE, pN, aT, K, cnM, eu, aW, kp, g, ac, bE, pw, ei, X); #c1(PALLD) c2(XP_011530070) c3(palladin isoform X1) c4( ) c5(325882, 325883, 325884, 325885, 325886) c6( ) c7(T, dD, aC, ea, e, aw, bB, j, eq, eJ, aE, pN, aT, K, cnM, eu, aW, kp, g, ac, bE, pw, ei, X); #c1(PALLD) c2(XP_011530071) c3(palladin isoform X2) c4(325887) c5(325888, 325889, 325890, 325891, 325892, 325893, 325894) c6( ) c7(T, dD, aC, ea, e, aw, bB, j, eq, eJ, aE, pN, aT, K, cnM, eu, aW, kp, g, ac, bE, pw, ei, X); #c1(PALLD) c2(XP_011530072) c3(palladin isoform X3) c4(325895) c5( ) c6( ) c7(T, dD, aC, ea, e, aw, bB, j, eq, eJ, aE, pN, aT, K, cnM, eu, aW, kp, g, ac, bE, pw, ei, X); #c1(PALLD) c2(XP_011530074) c3(palladin isoform X6) c4(325896) c5( ) c6( ) c7(T, dD, aC, ea, e, aw, bB, j, eq, eJ, aE, pN, aT, K, cnM, eu, aW, kp, g, ac, bE, pw, ei, X); #c1(PALLD) c2(XP_011530075) c3(palladin isoform X7) c4(325897) c5(325898) c6( ) c7(T, dD, aC, ea, e, aw, bB, j, eq, eJ, aE, pN, aT, K, cnM, eu, aW, kp, g, ac, bE, pw, ei, X); #c1(PALLD) c2(XP_011530076) c3(palladin isoform X8) c4( ) c5(325899, 325900, 325901, 325902, 325903, 325904, 325905, 325906, 325907, 325908, 325909, 325910, 325911, 325912) c6( ) c7(T, dD, aC, ea, e, aw, bB, j, eq, eJ, aE, pN, aT, K, cnM, eu, aW, kp, g, ac, bE, pw, ei, X); #c1(PALM2-AKAP2) c2(NP_009134) c3(PALM2-AKAP2 protein isoform 1) c4(171008) c5(171009, 171010, 171011, 171012, 171013, 171014, 171015, 171016, 171017, 171018) c6( ) c7(yn); #c1(PALM2-AKAP2) c2(NP_671492) c3(PALM2-AKAP2 protein isoform 2) c4(242021, 242022) c5(242023, 242024, 242025, 242026, 242027, 242028, 242029, 242030, 242031, 242032, 242033, 242034, 242035, 242036, 242037, 242038) c6( ) c7(yn); #c1(PAM) c2(NP_620177) c3(peptidyl-glycine alpha-amidating monooxygenase isoform d preproprotein) c4(237485) c5(237486, 237487, 237488, 237489, 237490, 237491) c6( ) c7(bL, aZy, n, dG, v, bjQ, eA, Jm, bv, av, aGm, ep, aT, aln, Fg, ae, bmo, uQ, hM, d, mR, aW, Y, fO, aZw, g, cm, bz, hq, ck, eM, dB, hR, ym, gT); #c1(PAM) c2(XP_006714695) c3(peptidyl-glycine alpha-amidating monooxygenase isoform X1) c4(287997) c5(287998, 287999, 288000, 288001, 288002, 288003) c6( ) c7(bL, aZy, n, dG, v, bjQ, eA, Jm, bv, av, aGm, ep, aT, aln, Fg, ae, bmo, uQ, hM, d, mR, aW, Y, fO, aZw, g, cm, bz, hq, ck, eM, dB, hR, ym, gT); #c1(PAM) c2(XP_006714697) c3(peptidyl-glycine alpha-amidating monooxygenase isoform X4) c4(288004) c5(288005, 288006, 288007, 288008, 288009, 288010) c6( ) c7(bL, aZy, n, dG, v, bjQ, eA, Jm, bv, av, aGm, ep, aT, aln, Fg, ae, bmo, uQ, hM, d, mR, aW, Y, fO, aZw, g, cm, bz, hq, ck, eM, dB, hR, ym, gT); #c1(PAM) c2(XP_006714699) c3(peptidyl-glycine alpha-amidating monooxygenase isoform X10) c4(288011) c5(288012, 288013, 288014, 288015, 288016, 288017) c6( ) c7(bL, aZy, n, dG, v, bjQ, eA, Jm, bv, av, aGm, ep, aT, aln, Fg, ae, bmo, uQ, hM, d, mR, aW, Y, fO, aZw, g, cm, bz, hq, ck, eM, dB, hR, ym, gT); #c1(PAM) c2(XP_006714701) c3(peptidyl-glycine alpha-amidating monooxygenase isoform X2) c4(288018, 288019) c5(288020, 288021, 288022, 288023, 288024, 288025) c6( ) c7(bL, aZy, n, dG, v, bjQ, eA, Jm, bv, av, aGm, ep, aT, aln, Fg, ae, bmo, uQ, hM, d, mR, aW, Y, fO, aZw, g, cm, bz, hq, ck, eM, dB, hR, ym, gT); #c1(PAM) c2(XP_006714703) c3(peptidyl-glycine alpha-amidating monooxygenase isoform X3) c4(288026) c5(288027, 288028, 288029, 288030, 288031, 288032) c6( ) c7(bL, aZy, n, dG, v, bjQ, eA, Jm, bv, av, aGm, ep, aT, aln, Fg, ae, bmo, uQ, hM, d, mR, aW, Y, fO, aZw, g, cm, bz, hq, ck, eM, dB, hR, ym, gT); #c1(PAM) c2(XP_01154/721) c3(peptidyl-glycine alpha-amidating monooxygenase isoform X5) c4( ) c5(340278, 340279, 340280, 340281, 340282, 340283, 340284) c6( ) c7(bL, aZy, n, dG, v, bjQ, eA, Jm, bv, av, aGm, ep, aT, aln, Fg, ae, bmo, uQ, hM, d, mR, aW, Y, fO, aZw, g, cm, bz, hq, ck, eM, dB, hR, ym, gT); #c1(PAM) c2(XP_011541723) c3(peptidyl-glycine alpha-amidating monooxygenase isoform X7) c4(340285) c5( ) c6( ) c7(bL, aZy, n, dG, v, bjQ, eA, Jm, bv, av, aGm, ep, aT, aln, Fg, ae, bmo, uQ, hM, d, mR, aW, Y, fO, aZw, g, cm, bz, hq, ck, eM, dB, hR, ym, gT); #c1(PAM) c2(XP_01154/725) c3 (peptidyl-glycine alpha-amidating monooxygenase isoform X9) c4(340286, 340287) c5(340288, 340289, 340290, 340291, 340292, 340293) c6( ) c7(bL, aZy, n, dG, v, bjQ, eA, Jm, bv, av, aGm, ep, aT, aln, Fg, ae, bmo, uQ, hM, d, mR, aW, Y, fO, aZw, g, cm, bz, hq, ck, eM, dB, hR, ym, gT); #c1(PAM) c2(XP_011541727) c3(peptidyl-glycine alpha-amidating monooxygenase isoform X12) c4(340294, 340295, 340296, 340297) c5( ) c6( ) c7(bL, aZy, n, dG, v, bjQ, eA, Jm, bv, av, aGm, ep, aT, aln, Fg, ae, bmo, uQ, hM, d, mR, aW, Y, fO, aZw, g, cm, bz, hq, ck, eM, dB, hR, ym, gT); #c1(PAM) c2(XP_011541729) c3(peptidyl-glycine alpha-amidating monooxygenase isoform X15) c4( ) c5(340298, 340299, 340300, 340301, 340302, 340303, 340304, 340305, 340306, 340307, 340308, 340309, 340310, 340311, 340312, 340313, 340314) c6( ) c7(bL, aZy, n, dG, v, bjQ, eA, Jm, bv, av, aGm, ep, aT, aln, Fg, ae, bmo, uQ, hM, d, mR, aW, Y, fO, aZw, g, cm, bz, hq, ck, eM, dB, hR, ym, gT); #c1(PAM16) c2(NP_057153) c3(mitochondrial import inner membrane translocase subunit TlM16) c4(189173) c5(189174, 189175, 189176, 189177, 189178, 189179) c6( ) c7(OH, yo, ad); #c1(PAMR1) c2(NP_001001991) c3 (inactive serine protease PAMRl isoform b precursor) c4(19279) c5(19280, 19281, 19282, 19283, 19284, 19285, 19286, 19287) c6(19288, 19289) c7(et, xs, aA, nf, uY, dz, aF); #c1(PAMR1) c2(NP_001269605) c3(inactive serine protease PAMR1 isoform d precursor) c4(102176) c5(102177, 102178, 102179, 102180, 102181, 102182, 102183, 102184) c6(102185, 102186) c7(et, xs, aA, nf, uY, dz, aF); #c1(PAMR1) c2(NP_056245) c3(inactive serine protease PAMR1 isoform a precursor) c4(186694) c5(186695, 186696, 186697, 186698, 186699, 186700, 186701, 186702) c6(186703, 186704) c7(et, xs, aA, nf, uY, dz, aF); #c1(PAN2) c2(NP_001120932) c3(PAB-dependent poly(A)-specific ribonuclease subunit PANZ isoform 1) c4(44952) c5(44953, 44954, 44955, 44956, 44957, 44958, 44959, 44960, 44961, 44962) c6( ) c7(T, q, r); #c1(PAN2) c2(NP_001159751) c3(PAB-dependent poly(A)-specific ribonuclease subunit PANZ isoform 2) c4(64706) c5(64707, 64708, 64709, 64710, 64711, 64712) c6( ) c7(T, q, r); #c1(PAN2) c2(NP_055686) c3(PAB-dependent poly(A)-specific ribonuclease subunit PAN2 isoform 3) c4(182768) c5(182769, 182770, 182771, 182772, 182773, 182774) c6( ) c7(T, q, r); #c1(PAN3) c2(NP_787050) c3(PAB-dependent poly(A)-specific ribonuclease subunit PAN3) c4(252012) c5(252013, 252014, 252015, 252016, 252017, 252018) c6( ) c7(aE, ac); #c1(PAN3) c2(XP_005266390) c3(PAB-dependent poly(A)-specific ribonuclease subunit PAN3 isoform X2) c4(279966) c5(279967, 279968, 279969, 279970, 279971, 279972) c6( ) c7(aE, ac); #c1(PAN3) c2(XP_005266391) c3(PAB-dependent poly(A)-specific ribonuclease subunit PAN3 isoform X7) c4(279973) c5(279974, 279975, 279976, 279977, 279978, 279979) c6( ) c7(aE, ac); #c1(PANK1) c2(NP_612189) c3(pantothenate kinase 1 isoform gamma) c4(236128) c5(236129, 236130, 236131, 236132, 236133, 236134) c6( ) c7(Pw, axa, K, U); #c1(PANK1) c2(NP_683878) c3 (pantothenate kinase 1 isoform alpha) c4(242616, 242617, 242618) c5(242619, 242620, 242621, 242622, 242623, 242624, 242625, 242626, 242627, 242628, 242629, 242630) c6( ) c7(Pw, axa, K, U); #c1(PANK1) c2(NP_683879) c3(pantothenate kinase 1 isoform beta) c4(242631) c5(242632, 242633, 242634, 242635, 242636, 242637, 242638, 242639, 242640, 242641, 242642, 242643) c6( ) c7(Pw, axa, K, U); #c1(PANK2) c2(NP_705902) c3(pantothenate kinase 2, mitochondrial isoform 1 preproprotein) c4(246410, 246411, 246412) c5(246413, 246414, 246415, 246416, 246417, 246418) c6( ) c7(axa, PW, Pv, Pw, aPR, HC, U, aAm, aU, K, xw, Pu); #c1(PANK2) c2(NP_705904) c3(pantothenate kinase 2, mitochondrial isoform 2) c4( ) c5(246419, 246420, 246421, 246422, 246423) c6( ) c7(axa, PW, Pv, Pw, aPR, HC, U, aAm, aU, K, xw, Pu); #c1(PANK2) c2(XP_005260892) c3(pantothenate kinase 2, mitochondrial isoform X3) c4( ) c5(276960, 276961, 276962, 276963, 276964, 276965, 276966) c6( ) c7(axa, PW, Pv, Pw, aPR, HC, U, aAm, aU, K, xw, Pu); #c1(PANX1) c2(NP_056183) c3(pannexin-1) c4(186178, 186179) c5(186180, 186181, 186182, 186183, 186184, 186185, 186186, 186187, 186188, 186189, 186190, 186191) c6( ) c7(Y, h, fe, ji, iV, t, eA); #c1(PANX2) c2(NP_443071) c3(pannexin-2 isoform 1) c4(229228, 229229) c5(229230, 229231, 229232, 229233, 229234, 229235, 229236, 229237, 229238, 229239, 229240, 229241) c6( ) c7(ac, eA, rH); #c1(PADX) c2(NP_690875) c3(peroxisomal N(1)-acetyl-spermine/spermidine oxidase isoform 1) c4(245034, 245035, 245036, 245037, 245038) c5(245039, 245040, 245041, 245042, 245043, 245044, 245045, 245046, 245047, 245048, 245049, 245050, 245051, 245052, 245053, 245054, 245055, 245056, 245057, 245058, 245059, 245060) c6( ) c7(ae, ac, aW, aT, ao, g, bz, ad); #c1(PAOX) c2(NP_997010) c3(peroxisomal N(1)-acetyl-spermine/spermidine oxidase isoform 2) c4(264956, 264957) c5(264958, 264959, 264960, 264961, 264962, 264963, 264964, 264965, 264966, 264967, 264968, 264969, 264970, 264971, 264972, 264973, 264974) c6( ) c7(ae, ac, aW, aT, ao, g, bz, ad); #c1(PAOX) c2(NP_997011) c3(peroxisomal N(1)-acetyl-spermine/spermidine oxidase isoform 4) c4(264975) c5(264976, 264977, 264978, 264979, 264980, 264981) c6( ) c7(ae, ac, aW, aT, ao, g, bz, ad); #c1(PAPD7) c2(XP_005248291) c3(non-canonical poly(A) RNA polymerase PAPD7 isoform X2) c4(268292) c5(268293, 268294, 268295, 268296, 268297, 268298, 268299) c6( ) c7(if, Be, r, bK, K, qN, bz, q); #c1(PAPD7) c2(XP_011512249) c3(non-canonical poly(A) RNA polymerase PAPD7 isoform X1) c4(302823) c5(302824, 302825, 302826, 302827, 302828) c6( ) c7(if, Be, r, bK, K, qN, bz, q); #c1(PAPL) c2(NP_001004318) c3(iron/zinc purple acid phosphatase-like protein precursor) c4(20566) c5(20567, 20568, 20569, 20570, 20571, 20572, 20573, 20574) c6(20575, 20576) c7(bL, B, vi); #c1(PAPOLA) c2(NP_001238935) c3(poly(A) polymerase alpha isoform 2) c4(88048) c5(88049, 88050, 88051, 88052, 88053, 88054) c6( ) c7(ck, d, dA, iW, r, wq, dT, aj, v, zG, uF, zQ, cd, ao); #c1(PAPOLA) c2(NP_001280556) c3(poly(A) polymerase alpha isoform 4) c4( ) c5(111523, 111524) c6( ) c7(ck, d, dA, iW, r, wq, dT, aj, v, zG, uF, zl, cd, ao); #c1(PAPOLA) c2(NP_116021) c3(poly(A) polymerase alpha isoform 1) c4(225232) c5(225233, 225234, 225235, 225236, 225237, 225238) c6( ) c7(ck, d, dA, iW, r, wq, dT, aj, v, zG, uF, zl, cd, ao); #c1(PAPOLA) c2(XP_005267338) c3(poly(A) polymerase alpha isoform X1) c4(280905) c5(280906, 280907, 280908, 280909, 280910, 280911) c6( ) c7(ck, d, dA, iW, r, wq, dT, aj, v, zG, uF, zl, cd, ao); #c1(PAPOLG) c2(NP_075045) c3(poly(A) polymerase gamma) c4(213550) c5(213551, 213552, 213553, 213554, 213555, 213556) c6( ) c7(uF, eV, T, ex, Y); #c1(PAPOLG) c2(XP_005264557) c3(poly(A) polymerase gamma isoform X1) c4(279047, 279048) c5(279049, 279050, 279051, 279052, 279053, 279054, 279055, 279056, 279057, 279058, 279059, 279060) c6( ) c7(uF, eV, T, eX, Y); #c1(PAPPA) c2(NP_002572) c3(pappalysin-1 preproprotein) c4(129873, 129874, 129875) c5(129876, 129877, 129878, 129879, 129880, 129881, 129882, 129883, 129884, 129885, 129886, 129887, 129888, 129889, 129890, 129891, 129892, 129893) c6( ) c7(gx, T, dD, eR, ea, aw, eb, n, j, eq, aY, cd, dP, aHF, Na, vJ, rS, aT, Nc, hH, Df, hm, cY, nO, aW, ace, eZ, fe, t, f, bz, op, sn, vA, gZ, aNL, cS, m, ER); #c1(PAPPA2) c2(NP_068755) c3(pappalysin-2 isoform 2 precursor) c4(210184) c5(210185, 210186, 210187, 210188, 210189, 210190, 210191, 210192) c6(210193, 210194) c7(T, jv, eb); #c1(PAPPA2) c2(XP_011508160) c3(pappalysin-2 isoform X1) c4(298654) c5(298655, 298656, 298657, 298658, 298659, 298660, 298661, 298662) c6(298663, 298664) c7(T, jv, eb); #c1(PAPPA2) c2(XP_011508161) c3(pappalysin-2 isoform X2) c4( ) c5(298665, 298666) c6( ) c7(T, jv, eb); #c1(PAPSS1) c2(NP_005434) c3(bifunctional 3′-phosphoadenosine 5′-phosphosulfate synthase 1) c4(156144) c5(156145, 156146, 156147, 156148, 156149, 156150) c6( ) c7(gk, T, ac, aT, aq, j, B, hX); #c1(PAPSS2) c2(NP_001015880) c3(bifunctional 3′-phosphoadenosine 5′-phosphosulfate synthase 2 isoform b) c4(26214, 26215) c5(26216, 26217, 26218, 26219, 26220, 26221, 26222, 26223, 26224, 26225, 26226, 26227) c6( ) c7(vK, aZ, ln, cnR, cnP, cnN, nA, cnQ, rv, cnO); #c1(PAPSS2) c2(NP_004661) c3(bifunctional 3′-phosphoadenosine 5′-phosphosulfate synthase 2 isoform a) c4(148611, 148612) c5(148613, 148614, 148615, 148616, 148617, 148618, 148619, 148620, 148621, 148622, 148623, 148624) c6( ) c7(vK, aZ, ln, cnR, cnP, cnN, nA, cnQ, rv, cnO); #c1(PAAR3) c2(NP_001035292) c3(progestin and adipod receptor family member 3) c4(32748) c5(32749, 32750, 32751, 32752, 32753, 32754, 32755, 32756, 32757) c6( ) c7(bF, yk, Y, fc, r, v, cP, q); #c1(PAQR3) c2(XP_006714169) c3(progestin and adipod receptor family member 3 isoform X1) c4(287622) c5(287623, 287624, 287625, 287626, 287627, 287628, 287629) c6( ) c7(bF, yk, Y, fc, r, v, cP, q); #c1(PAQR3) c2(XP_006714170) c3(progestin and adipol receptor family member 3 isoform X2) c4(287630) c5(287631, 287632, 287633, 287634, 287635) c6( ) c7(bF, yk, Y, fc, r, v, cP, q); #c1(PAQR5) c2(NP_060175) c3(membrane progestin receptor gamma) c4(194619) c5(194620, 194621, 194622, 194623, 194624, 194625, 194626, 194627, 194628, 194629, 194630, 194631, 194632, 194633) c6( ) c7(he, dY); #c1(PAAR7) c2(XP_011539164) c3(membrane progestin receptor alpha isoform X1) c4(337271) c5(337272, 337273, 337274, 337275, 337276, 337277) c6( ) c7(dD, ea, eq, ae, dr, ad); #c1(PARD3) c2(NP_001171714) c3(partitioning defective 3 homolog isoform 2) c4(71396) c5(71397, 71398, 71399, 71400, 71401, 71402) c6( ) c7(sc, T, fG, yk, iW, fr, ci, ad, bv, aT, hn, wb, ae, d, s, Y, ff, cr, hp, en, iV, f, LB, sS, Dy, vA, hE, it, B); #c1(PARD3) c2(NP_001171715) c3(partitioning defective 3 homolog isoform 3) c4(71403) c5(71404, 71405, 71406, 71407, 71408, 71409) c6( ) c7(sc, T, fG, yk, iW, fr, ci, ad, bv, aT, hn, wb, ae, d, s, Y, ff, cr, hp, en, iV, f, LB, sS, Dy, vA, hE, it, B); #c1(PARD3) c2(NP_001171716) c3 (partitioning defective 3 homolog isoform 4) c4(71410) c5(71411, 71412, 71413, 71414, 71415, 71416) c6( ) c7(sc, T, fG, yk, iW, fr, ci, ad, bv, aT, hn, wb, ae, d, s, Y, ff, cr, hp, en, iV, f, LB, sS, Dy, vA, hE, it, B); #c1(PARD3) c2(NP_001171717) c3(partitioning defective 3 homolog isoform 5) c4(71417) c5(71418, 71419, 71420, 71421, 71422, 71423) c6( ) c7(sc, T, fG, yk, iW, fr, ci, ad, bv, aT, hn, wb, ae, d, s, Y, ff, cr, hp, en, iV, f, LB, sS, Dy, vA, hE, it, B); #c1(PARD3) c2(NP_001171719) c3(partitioning defective 3 homolog isoform 7) c4(71424) c5(71425, 71426, 71427, 71428, 71429, 71430) c6( ) c7(sc, T, fG, yk, iW, fr, ci, ad, bv, aT, hn, wb, ae, d, s, Y, ff, cr, hp, en, iV, f, LB, sS, Dy, vA, hE, it, B); #c1(PARD3) c2(NP_001171720) c3(partitioning defective 3 homolog isoform 8) c4(71431) c5(71432, 71433, 71434, 71435, 71436, 71437) c6( ) c7(sc, T, fG, yk, iW, fr, ci, ad, bv, aT, hn, wb, ae, d, s, Y, ff, cr, hp, en, iV, f, LB, sS, Dy, vA, hE, it, B); #c1(PARD3) c2(NP_001171721) c3 (partitioning defective 3 homolog isoform 9) c4(71438) c5(71439, 71440, 71441, 71442, 71443, 71444) c6( ) c7(sc, T, fG, yk, iW, fr, ci, ad, bv, aT, hn, wb, ae, d, s, Y, ff, cr, hp, en, iV, f, LB, sS, Dy, vA, hE, it, B); #c1(PARD3) c2(NP_001171723) c3(partitioning defective 3 homolog isoform 11) c4(71445) c5(71446, 71447, 71448, 71449, 71450, 71451) c6( ) c7(sc, T, fG, yk, iW, fr, ci, ad, bv, aT, hn, wb, ae, d, s, Y, ff, cr, hp, en, iV, f, LB, sS, Dy, vA, hE, it, B); #c1(PARD3) c2(NP_062565) c3(partitioning defective 3 homolog isoform 1) c4(201998) c5(201999, 202000, 202001, 202002, 202003, 202004) c6( ) c7(sc, T, fG, yk, iW, fr, ci, ad, bv, aT, hn, wb, ae, d, s, Y, ff, cr, hp, en, iV, f, LB, sS, Dy, vA, hE, it, B); #c1(PARD3) c2(XP_005252588) c3(partitioning defective 3 homolog isoform X9) c4(271889) c5(271890, 271891, 271892, 271893, 271894, 271895) c6( ) c7(sc, T, fG, yk, iW, fr, ci, ad, bv, aT, hn, wb, ae, d, s, Y, ff, cr, hp, en, iV, f, LB, sS, Dy, vA, hE, it, B); #c1(PARD3) c2(XP_005252589) c3(partitioning defective 3 homolog isoform X12) c4(271896) c5(271897, 271898, 271899, 271900, 271901, 271902) c6( ) c7(sc, T, fG, yk, iW, fr, ci, ad, bv, aT, hn, wb, ae, d, s, Y, ff, cr, hp, en, iV, f, LB, sS, Dy, vA, HE, it, B); #c1(PARD3) c2(XP_005252592) c3(partitioning defective 3 homolog isoform X21) c4(271903) c5(271904, 271905, 271906, 271907, 271908, 271909) c6( ) c7(sc, T, fG, yk, iW, fr, ci, ad, bv, aT, hn, wb, ae, d, s, Y, ff, cr, hp, en, iV, f, LB, sS, Dy, vA, hE, it, B); #c1(PARD3) c2(XP_005252593) c3 (partitioning defective 3 homolog isoform X23) c4( ) c5(271910, 271911, 271912, 271913, 271914, 271915) c6( ) c7(sc, T, fG, yk, iW, fr, ci, ad, bv, aT, hn, wb, ae, d, s, Y, ff, cr, hp, en, iV, f, LB, sS, Dy, vA, hE, it, B); #c1(PARD3) c2(XP_011517877) c3 (partitioning defective 3 homolog isoform X10) c4(311789) c5( ) c6( ) c7(sc, T, fG, yk, iW, fr, ci, ad, bv, aT, hn, wb, ae, d, s, Y, ff, cr, hp, en, iV, f, LB, sS, Dy, vA, hE, it, B); #c1(PARD3B) c2(NP_001289698) c3(partitioning defective 3 homolog B isoform Par3La) c4(114367) c5(114368, 114369, 114370, 114371, 114372, 114373) c6( ) c7(cF, v, k); #c1(PARD3B) c2(NP_476518) c3(partitioning defective 3 homolog B isoform Par3Lc) c4(230736) c5(230737, 230738, 230739, 230740, 230741, 230742) c6( ) c7(cF, v, k); #c1(PARD3B) c2(NP_689739) c3(partitioning defective 3 homolog B isoform Par3Lb) c4(243809) c5(243810, 243811, 243812, 243813, 243814, 243815) c6( ) c7(cF, v, k); #c1(PARD3B) c2(NP_995585) c3(partitioning defective 3 homolog B isoform 4) c4(263964) c5(263965, 263966, 263967, 263968, 263969, 263970) c6( ) c7(cF, v, k); #c1(PARDGA) c2(NP_001032358) c3(partitioning defective 6 homolog alpha isoform 2) c4(31240) c5(31241, 31242, 31243, 31244, 31245, 31246, 31247) c6( ) c7(eM, Mz, y, nJ, bz, af); #c1(PARDEA) c2(NP_058644) c3 (partitioning defective 6 homolog alpha isoform 1) c4(192942) c5(192943, 192944, 192945, 192946, 192947, 192948) c6( ) c7(eM, Mz, y, nJ, bz, af); #c1(PARDGB) c2(NP_115910) c3(partitioning defective 6 homolog beta) c4(224636) c5(224637, 224638, 224639, 224640, 224641, 224642) c6( ) c7(bK); #c1(PARG) c2(NP_001290415) c3(poly(ADP-ribose) glycohydrolase isoform b) c4(115389) c5( ) c6( ) c7(eW, T, wg, bE, aW, iW, aT, JT, aq, SH, K, eA); #c1(PARG) c2(NP_001290416) c3(poly(ADP-ribose) glycohydrolase isoform c) c4( ) c5(115390, 115391, 115392, 115393, (15394, 115395) c6( ) c7(eW, T, wg, bE, aW, iW, aT, JT, aq, SH, K, eA); #c1(PARG) c2(NP_003622) c3(poly(ADP-ribose) glycohydrolase isoform a) c4(139694) c5( ) c6( ) c7(eW, T, wg, bE, aW, iW, aT, JT, aq, SH, K, eA); #c1(PARG) c2(XP_011538611) c3(poly(ADP-ribose) glycohydrolase isoform X5) c4( ) c5(336315, 336316, 336317, 336318, 336319, 336320) c6( ) c7(eW, T, wg, bE, aW, iW, aT, JT, aq, SH, K, eA); #c1(PARK2) c2(NP_004553) c3(E3 ubiquitin-protein ligase parkin isoform 1) c4(147595, 147596, 147597) c5(147598, 147599, 147600, 147601, 147602, 147603, 147604) c6( ) c7(T, os, aC, yS, al, bB, MF, ci, aY, aMB, x, AW, aji, Gt, aE, rw, aU, K, bBp, bU, cnS, Jl, aW, Ls, aiT, bw, jV, eM, iA, iT, U, nw, k, m, act, iu, dt, Ri, aj, BF, fr, nA, eW, Hx, aMV, h, yT, yZ, bl, bLD, pi, QP, aq, fq, hq, agl, art, B, bL, fM, cz, GC, v, j, bAM, C, bX, yU, q, J, eA, oT, bgk, lg, HC, bUn, aT, jj, an, ae, alL, ak, iU, Y, PL, bTw, r, lo, t, xw, bN, dS, ck, bAn, alC, aWe, bt, bAr, VB, cnT, hl, jh, oZ, xt, ad, hX, av, py, ot, pq, d, cr, nf, dq, Tp, iR, bz, GB, bF, es, bE, aS, y, kS, X, jr); #c1(PARK2) c2(NP_054642) c3(E3 ubiquitin-protein ligase parkin isoform 2) c4(177266) c5(177267, 177268, 177269, 177270, 177271, 177272) c6( ) c7(T, os, aC, yS, al, bB, MF, ci, aY, aMB, x, AW, aji, Gt, aE, rw, aU, K, bBp, bU, cnS, Jl, aW, Ls, aiT, bw, jV, eM, iA, iT, U, nw, k, m, act, iu, dt, Ri, aj, BF, fr, nA, eW, Hx, aMV, h, yT, yZ, bl, bLD, pi, QP, aq, fq, hq, agl, art, B, bL, fM, cz, GC, v, j, bAM, C, bX, yU, q, J, eA, oT, bgk, lg, HC, bUn, aT, jj, an, ae, alL, ak, iU, Y, PL, bTw, r, lo, t, xw, bN, dS, ck, bAn, alC, aWe, bt, bAr, VB, cnT, hl, jh, oZ, xt, ad, hX, av, py, ot, pq, d, cr, nf, dq, Tp, iR, bz, GB, bF, es, bE, aS, y, kS, X, jr); #c1(PARK2) c2(NP_054643) c3(E3 ubiquitin-protein ligase parkin isoform 3) c4(177273) c5(177274, 177275, 177276, 177277, 177278, (77279) c6( ) c7(T, os, aC, yS, aT, bB, MF, ci, aY, aMB, x, AW, aji, Gt, aE, rw, aU, K, bBp, bU, cnS, Jl, aW, Ls, aiT, bw, jV, eM, iA, iT, U, nw, k, m, act, iu, dt, Ri, aj, BF, fr, nA, eW, Hx, aMV, h, yT, yZ, bl, bLD, pi, QP, aq, fq, hq, agl, art, B, bL, fM, cz, GC, v, j, bAM, C, bX, yU, q, J, eA, oT, bgk, lg, HC, bUn, aT, j, an, ae, alL, ak, iU, Y, PL, bTw, r, lo, t, xw, bN, dS, ck, bAn, alC, aWe, bt, bAr, VB, cnT, hl, jh, oZ, xt, ad, hX, av, py, ot, pq, d, cr, nf, dq, Tp, iR, bz, GB, bF, es, bE, aS, y, kS, X, jr); #c1(PARK2) c2(XP_011534165) c3(E3 ubiquitin-protein ligase parkin isoform X1) c4(330436) c5(330437, 330438, 330439, 330440, 330441, 330442, 330443, 330444, 330445, 330446, 330447, 330448, 330449, 330450, 330451) c6( ) c7(T, os, aC, yS, al, bB, MF, ci, aY, aMB, x, AW, aji, Gt, aE, rw, aU, K, bBp, bU, cnS, Jl, aW, Ls, aiT, bw, jV, eM, iA, iT, U, nw, k, m, act, iu, dt, Ri, aj, BF, fr, nA, eW, Hx, aMV, h, yT, yZ, bl, bLD, pi, QP, aq, fq, hq, agl, art, B, bL, fM, cz, GC, v, j, bAM, C, bX, yU, q, J, eA, oT, bgk, lg, HC, bUn, aT, jj, an, ae, alL, ak, iU, Y, PL, bTw, r, lo, t, xw, bN, dS, ck, bAn, alC, aWe, bt, bAr, VB, cnT, hl, jh, oZ, xt, ad, hX, av, py, ot, pq, d, cr, nf, dq, Tp, iR, bz, GB, bF, es, bE, aS, y, kS, X, jr); #c1(PARK2) c2(XP_011534166) c3(E3 ubiquitin-protein ligase parkin isoform X2) c4(330452, 330453) c5(330454, 330455, 330456, 330457, 330458, 330459) c6( ) c7(T, os, aC, yS, al, bB, MF, ci, aY, aMB, x, AW, aji, Gt, aE, rw, aU, K, bBp, bU, cnS, Jl, aW, Ls, aiT, bw, jV, eM, iA, iT, U, nw, k, m, act, iu, dt, Ri, aj, BF, fr, nA, eW, Hx, aMV, h, yT, yZ, bl, bLD, pi, QP, aq, fq, hq, agl, art, B, bL, fM, cz, GC, v, j, bAM, C, bX, yU, q, J, eA, oT, bgk, lg, HC, bUn, aT, j, an, ae, alL, ak, iU, Y, PL, bTw, r, lo, t, xw, bN, dS, ck, bAn, alC, aWe, bt, bAr, VB, cnT, hl, jh, oZ, xt, ad, hX, av, py, ot, pq, d, cr, nf, dq, Tp, iR, bz, GB, bF, es, bE, aS, y, kS, X, jr); #c1(PARK2) c2(XP_011534167) c3(E3 ubiquitin-protein ligase parkin isoform X3) c4( ) c5(330460, 330461, 330462, 330463, 330464, 330465) c6( ) c7(T, os, aC, yS, aT, bB, MF, ci, aY, aMB, x, AW, aji, Gt, aE, rw, aT, K, bBp, bU, cnS, Jl, aW, Ls, aiT, bw, jV, eM, iA, iT, U, nw, k, m, act, iu, dt, Ri, aj, BF, fr, nA, eW, Hx, aMV, h, yT, yZ, bl, bLD, pi, QP, aq, fq, hq, agl, art, B, bL, fM, cz, GC, v, j, bAM, C, bX, yU, q, J, eA, oT, bgk, lg, HC, bUn, aT, jj, an, ae, alL, ak, iU, Y, PL, bTw, r, lo, t, xw, bN, dS, ck, bAn, alC, aWe, bt, bAr, VB, cnT, hl, jh, oZ, xt, ad, hX, av, py, ot, pq, d, cr, nf, dq, Tp, iR, bz, GB, bF, es, bE, aS, y, kS, X, jr); #c1(PARK2) c2(XP_011534169) c3(E3 ubiquitin-protein ligase parkin isoform X5) c4( ) c5(330466, 330467, 330468, 330469, 330470, 330471) c6( ) c7(T, os, aC, yS, aT, bB, MF, ci, aY, aMB, x, AW, aji, Gt, aE, rw, aU, K, bBp, bU, cnS, Jl, aW, Ls, aiT, bw, jV, eM, iA, iT, U, nw, k, m, act, iu, dt, Ri, aj, BF, fr, nA, eW, Hx, aMV, h, yT, yZ, bl, bLD, pi, QP, aq, fq, hq, agl, art, B, bL, fM, cz, GC, v, j, bAM, C, bX, yU, q, J, eA, oT, bgk, lg, HC, bUn, aT, jj, an, ae, aU, ak, iU, Y, PL, bTw, r, lo, t, xw, bN, dS, ck, bAn, alC, aWe, bt, bAr, VB, cnT, hl, jh, oZ, xt, ad, hX, av, py, ot, pq, d, cr, nf, dq, Tp, iR, bz, GB, bF, es, bE, aS, y, kS, X, jr); #c1(PARL) c2(NP_001032728) c3(presenilins-associated rhomboid-like protein, mitochondrial isoform 2 preproprotein) c4(31447) c5(31448, 31449, 31450, 31451, 31452, 31453) c6( ) c7(bL, pZ, j, eL, ei, K); #c1(PARL) c2(NP_061092) c3(presenilins-associated rhomboid-like protein, mitochondrial isoform 1 preproprotein) c4(199144) c5(199145, 199146, 199147, 199148, 199149, 199150) c6( ) c7(bL, pZ, j, eQ, ei, K); #c1(PARM1) c2(NP_056208) c3(prostate androgen-regulated mucin-like protein 1 precursor) c4(186333) c5(186334, 186335, 186336, 186337, 186338, 186339) c6( ) c7(ae, dt, ad, Jh); #c1(PARN) c2(NP_001127949) c3(poly(A)-specific ribonuclease PARN isoform 2) c4(49539) c5(49540, 49541, 49542, 49543, 49544, 49545, 49546, 49547, 49548, 49549, 49550) c6( ) c7(cm, T, aE); #c1(PARN) c2(NP_001229921) c3(poly(A)-specific ribonuclease PARN isoform 3) c4(85513) c5(85514, 85515, 85516, 85517, 85518, 85519) c6( ) c7(cm, T, aE); #c1(PARN) c2(NP_002573) c3(poly(A)-specific ribonuclease PARN isoform 1) c4(129894, 129895) c5(129896, 129897, 129898, 129899, 129900, 129901, 129902) c6( ) c7(cm, T, aE); #c1(PARN) c2(XP_011520814) c3(poly(A)-specific ribonuclease PARN isoform X3) c4(315810) c5( ) c6( ) c7(cm, T, aE); #c1(PARN) c2(XP_011520816) c3(poly(A)-specific ribonuclease PARN isoform X5) c4( ) c5(315811, 315812, 315813, 315814, 315815, 315816) c6( ) c7(cm, T, aE); #c1(PARP1) c2(NP_001609) c3(poly [ADP-ribose] polymerase 1) c4(119153, 119154, 119155, 119156) c5(119157, 119158, 119159, 119160, 119161, 119162, 119163, 119164, 119165, 119166, 119167, 119168, 119169, 119/70, 119171, 119172, 119173, 119174, 119175, 119176, 119177, 119178, 119179, 119180, 119181, 119182, 119183, 119184) c6( ) c7(bM, T, os, fR, pf, if, fd, bB, qj, ci, aR, ip, x, bv, aE, Qk, K, hu, pk, eL, cp, hm, aW, uY, pD, zC, g, cnV, eF, cm, bw, axG, y, fT, eM, nB, Fh, dc, iT, aiM, cj, Zn, nw, k, m, yA, abh, de, cZ, dD, ajm, sw, aw, ht, NT, n, aj, fr, nA, uE, qN, cd, br, zo, ai, eW, ke, ft, no, pe, cc, h, lG, aYf, hn, dL, apD, dd, asT, hB, dx, pl, Nr, FZ, bux, OV, asp, A, Yd, Tc, hQ, aq, po, kp, cM, op, oU, W, dB, hk, eD, hL, aar, at, fL, WL, it, B, cnW, bL, DM, fM, dA, ew, aA, v, j, C, bX, V, q, J, eA, eJ, uT, hW, afQ, rS, yg, aT, cy, an, ae, af, eu, CB, qw, Uf, wr, s, vT, iU, Y, r, en, cl, t, f, zJ, ck, Ta, iM, kP, c, Dy, ac, hc, aeM, dy, fD, bt, LS, hS, gV, kh, iW, jh, Hn, oZ, bS, dG, eq, mC, ad, hX, fU, av, hd, le, nl, abq, oW, dm, io, PZ, NM, vL, auY, d, je, hG, afF, ff, xs, eH, eZ, dN, aF, bz, ek, Hp, Nq, bF, bE, afd, Lb, bC, hE, cnU, bND, pC); #c1(PARP(2) c2(NP_073587) c3(poly [ADP-ribose] polymerase (2) c4(212980, 212981) c5(212982, 212983, 212984, 212985, 212986, 212987, 212988, 212989, 212990, 212991, 212992, 212993) c6( ) c7(ds, lo, K); #c1(PARP14) c2(NP_060024) c3(poly [ADP-ribose] polymerase 14) c4(193744, 193745, 193746, 193747, 193748) c5(193749, 193750, 193751, 193752, 193753, 193754, 193755, 193756, 193757, 193758, 193759, 193760, 193761, 193762, 193763, 193764, 193765, 193766, 193767, 193768, 193769, 193770, 193771, 193772) c6( ) c7(fd); #c1(PARP14) c2(XP_011511231) c3(poly [ADP-ribose] polymerase 14 isoform X2) c4( ) c5(301713, 301714, 301715, 301716, 301717, 301718) c6( ) c7(fd); #c1(PARP15) c2(NP_001106995) c3(poly [ADP-ribose] polymerase 15 isoform 1) c4(42764) c5(42765, 42766, 42767, 42768, 42769, 42770) c6( ) c7(aj); #c1(PARP15) c2(NP_689828) c3(poly [ADP-ribose] polymerase 15 isoform 2 precursor) c4(244058) c5(244059, 244060, 244061, 244062, 244063, 244064) c6( ) c7(aj); #c1(PARP15) c2(XP_011510781) c3 (poly [ADP-ribose] polymerase 15 isoform X7) c4(301186) c5(301187, 301188, 301189, 301190, 301191, 301192) c6( ) c7(aj); #c1(PARP2) c2(NP_001036083) c3(poly [ADP-ribose] polymerase 2 isoform 2) c4(33956) c5(33957, 33958, 33959, 33960, 33961, 33962) c6( ) c7(T, aW, aT, vH, m, bX, V, GR); #c1(PARP2) c2(NP_005475) c3(poly [ADP-ribose] polymerase 2 isoform 1) c4(156507) c5(156508, 156509, 156510, 156511, 156512, 156513) c6( ) c7(T, aW, aT, vH, m, bX, V, GR); #c1(PARP3) c2(NP_001003931) c3(poly [ADP-ribose] polymerase 3 isoform a) c4(20292, 20293) c5(20294, 20295, 20296, 20297, 20298, 20299, 20300, 20301, 20302) c6( ) c7(b, dD, ea, dl, eq, i, cm, ao); #c1(PARP3) c2(NP_005476) c3(poly [ADP-ribose] polymerase 3 isoform b) c4(156514) c5(156515, 156516, 156517, 156518, 156519, 156520, 156521, 156522, 156523, 156524, 156525, 156526, 156527, 156528) c6( ) c7(b, dD, ea, dL, eq, i, cm, ao); #c1(PARP4) c2(NP_006428) c3(poly [ADP-ribose] polymerase 4) c4(165133) c5(165134, 165135, 165136, 165137, 165138, 165139, 165140) c6( ) c7(ck, T, B, jh, aA, at, cy, aq, dL, j, m, an, f, t, X); #c1(PARP6) c2(NP_064599) c3(poly [ADP-ribose] polymerase 6) c4(203217, 203218, 203219) c5(203220, 203221, 203222, 203223, 203224, 203225, 203226, 203227, 203228, 203229, 203230, 203231, 203232, 203233, 203234, 203235) c6( ) c7(dy, q, r); #c1(PARP6) c2(XP_005254614) c3(poly [ADP-ribose] polymerase 6 isoform X1) c4(273279) c5(273280, 273281, 273282, 273283, 273284, 273285) c6( ) c7(dy, q, r); #c1(PARP6) c2(XP_005254615) c3(poly [ADP-ribose] polymerase 6 isoform X2) c4( ) c5(273286) c6( ) c7(dy, q, r); #c1(PARP6) c2(XP_005254616) c3(poly [ADP-ribose] polymerase 6 isoform X3) c4(273287) c5(273288, 273289, 273290, 273291, 273292, 273293) c6( ) c7(dy, q, r); #c1(PARP6) c2(XP_011520108) c3(poly [ADP-ribose] polymerase 6 isoform X5) c4( ) c5(314998, 314999, 315000, 315001, 315002, 315003) c6( ) c7(dy, q, r); #c1(PARP6) c2(XP_011520109) c3(poly [ADP-ribose] polymerase 6 isoform X6) c4(315004) c5(315005, 315006, 315007, 315008, 315009) c6( ) c7(dy, q, r); #c1(PARP9) c2(NP_001139574) c3(poly [ADP-ribose] polymerase 9 isoform a) c4(59497) c5(59498, 59499, 59500, 59501, 59502, 59503, 59504, 59505, 59506, 59507, 59508, 59509) c6( ) c7(oO, bM, T, fM, fG, df, hF, iX, bW, h, vB, K, zy, MG, qE, ul, hZ, aky, aKD, aq, anW, pb, dr, LB, ok, ig, dc, dO, uR, cj, at, Br, dT, fD, B); #c1(PARP9) c2(NP_001139576) c3(poly [ADP-ribose] polymerase 9 isoform b) c4(59510, 59511) c5(59512, 59513, 59514, 59515, 59516, 59517, 59518, 59519, 59520, 59521) c6( ) c7(oO, bM, T, fM, fG, df, hF, iX, bW, h, vB, K, zy, MG, qE, ul, hZ, aKy, aKD, aq, anW, pb, dr, LB, ok, ig, dc, do, uR, cj, at, Br, dT, fD, B); #c1(PARP9) c2(NP_001139578) c3(poly [ADP-ribose] polymerase 9 isoform c) c4(59522) c5(59523, 59524, 59525, 59526, 59527, 59528) c6( ) c7(oO, bM, T, fM, fG, df, hF, iX, bW, h, vB, K, zy, MG, qE, ul, hZ, aKy, aKD, aq, anW, pb, dr, LB, ok, ig, dc, dO, uR, cj, at, Br, dT, fD, B); #c1(PARPBP) c2(XP_011536808) c3(PCNA-interacting partner isoform X1) c4(334487, 334488) c5(334489, 334490, 334491, 334492, 334493, 334494, 334495, 334496) c6( ) c7(aeQ, T, os, gU, yi, aC, aE, bSU, yo, oZ, kp, HR, ey, pt, JO); #c1(PARPBP) c2(XP_011536816) c3(PCNA-interacting partner isoform X5) c4( ) c5(334497, 334498, 334499, 334500, 334501, 334502, 334503, 334504) c6( ) c7(aeQ, T, os, gU, yi, aC, aE, bSU, yo, oZ, kp, HR, ey, pt, JO); #c1(PARPBP) c2(XP_011536820) c3(PCNA-interacting partner isoform X12) c4(334505) c5(334506, 334507, 334508, 334509, 334510, 334511) c6( ) c7(aeQ, T, os, gU, yi, aC, aE, bSU, yo, oZ, kp, HR, ey, pt, JO); #c1(PARS2) c2(XP_011539505) c3(probable proline-tRNA ligase, mitochondrial isoform X1) c4( ) c5(337700, 337701, 337702, 337703, 337704, 337705) c6( ) c7(ac); #c1(PARVA) c2(NP_060692) c3(alpha-parvin) c4(197191, 197192, 197193, 197194) c5(197195, 197196, 197197, 197198, 197199, 197200, 197201, 197202, 197203, 197204, 197205, 197206, 197207, 197208, 197209, 197210, 197211, 197212, 197213) c6( ) c7(lB, T, cd); #c1(PARVA) c2(XP_005253072) c3(alpha-parvin isoform X1) c4( ) c5(272133, 272134, 272135, 272136, 272137, 272138) c6( ) c7(lB, T, cd); #c1(PARVB) c2(NP_001003828) c3(beta-parvin isoform a) c4(20220) c5(20221, 20222, 20223, 20224, 20225, 20226) c6( ) c7(adu, bM, T, aW, aT, iy, j, ci); #c1(PARVB) c2(NP_001230314) c3(beta-parvin isoform c) c4(86223) c5(86224, 86225, 86226, 86227, 86228, 86229) c6( ) c7(adu, bM, T, aW, aT, iy, j, ci); #c1(PARVB) c2(NP_037459) c3(beta-parvin isoform b) c4(175626) c5(175627, 175628, 175629, 175630, 175631, 175632) c6( ) c7(adu, bM, T, aW, aT, iy, j, ci); #c1(PARVG) c2(NP_001131077) c3(gamma-parvin) c4(52586, 52587) c5(52588, 52589, 52590, 52591, 52592, 52593, 52594, 52595, 52596, 52597, 52598) c6( ) c7(T, Fc); #c1(PARVG) c2(XP_011528604) c3(gamma-parvin isoform X3) c4( ) c5(323856, 323857, 323858) c6( ) c7(T, Fc); #c1(PARVG) c2(XP_011528609) c3(gamma-parvin isoform X6) c4(323859, 323860) c5(323861, 323862, 323863, 323864, 323865, 323866, 323867, 323868, 323869, 323870, 323871, 323872, 323873, 323874, 323875, 323876, 323877, 323878) c6( ) c7(T, Fc); #c1(PASD1) c2(NP_775764) c3(PAS domain-containing protein 1) c4(250222) c5(250223, 250224, 250225, 250226, 250227, 250228) c6( ) c7(kk, sS, fd, jh, bu); #c1(PASK) c2(NP_001239048) c3(PAS domain-containing serine/threonine-protein kinase isoform 1) c4(88219) c5(88220, 88221, 88222, 88223, 88224) c6( ) c7(bri, aj, K, bX, J, bN); #c1(PASK) c2(NP_001239049) c3(PAS domain-containing serine/threonine-protein kinase isoform 2) c4(88225, 88226) c5(88227, 88228, 88229, 88230, 88231, 88232, 88233, 88234, 88235, 88236, 88237, 88238, 88239, 88240) c6( ) c7(bri, aj, K, bX, J, bN); #c1(PASK) c2(NP_001239053) c3(PAS domain-containing serine/threonine-protein kinase isoform 4) c4(88241) c5(88242, 88243, 88244, 88245, 88246, 88247, 88248, 88249, 88250, 88251, 88252) c6( ) c7(bri, aj, K, bX, J, bN); #c1(PASK) c2(XP_011509128) c3(PAS domain-containing serine/threonine-protein kinase isoform X2) c4( ) c5(299751) c6( ) c7(bri, aj, K, bX, J, bN); #c1(PASK) c2(XP_011509129) c3(PAS domain-containing serine/threonine-protein kinase isoform X3) c4(299752) c5(299753, 299754, 299755) c6( ) c7(bri, aj, K, bX, J, bN); #c1(PASK) c2(XP_011509134) c3(PAS domain-containing serine/threonine-protein kinase isoform X5) c4(299756) c5(299757, 299758, 299759, 299760, 299761, 299762) c6( ) c7(bri, aj, K, bX, J, bN); #c1(PATE1) c2(NP_612151) c3(prostate and testis expressed protein 1 precursor) c4(236071, 236072) c5(236073, 236074, 236075, 236076, 236077, 236078, 236079, 236080, 236081, 236082, 236083, 236084, 236085, 236086, 236087, 236088) c6(236089, 236090, 236091, 236092) c7(ae, ad); #c1(PATZ1) c2(NP_055138) c3(POZ-, AT hook-, and zinc finger-containing protein 1 long C isoform) c4(179234) c5(179235, 179236, 179237, 179238, 179239, 179240, 179241) c6( ) c7(T, dD, wu, ea, jh, vH, eq, wm, bu, aiG); #c1(PATZ1) c2(NP_114439) c3(POZ-, AT hook-, and zinc finger-containing protein l long A isoform) c4(222802) c5(222803, 222804, 222805, 222806, 222807, 222808) c6( ) c7(T, dD, wu, ea, jh, vH, eq, wm, bu, aiG); #c1(PATZ1) c2(NP_114440) c3(POZ-, AT hook-, and zinc finger-containing protein 1 short isoform) c4(222809) c5(222810, 222811, 222812, 222813, 222814, 222815) c6( ) c7(T, dD, wu, ea, jh, vH, eq, wm, bu, aiG); #c1(PATZ1) c2(NP_114441) c3(POZ-, AT hook-, and zinc finger-containing protein l long B isoform) c4(222816) c5(222817, 222818, 222819, 222820, 222821, 222822) c6( ) c7(T, dD, wu, ea, jh, vH, eq, wm, bu, aiG); #c1(PAWR) c2(XP_006719498) c3(PRKC apoptosis WTl regulator protein isoform X1) c4(292575, 292576, 292577) c5(292578, 292579, 292580, 292581, 292582, 292583, 292584) c6( ) c7(gh, T, aC, fG, bO, bB, bS, ct, ci, eA, ad, av, jz, aT, h, K, an, H, he, ae, xa, af, CB, jf, Uf, B, aW, Y, auM, eH, g, cl, Z, eF, cm, bz, ek, hq, ck, ac, c, bE, wO, acS, apl, yP, U, hL, lF, dX, alv, EY, X, L); #c1(PAWR) c2(XP_006719499) c3(PRKC apoptosis WTl regulator protein isoform X2) c4( ) c5(292585, 292586, 292587, 292588, 292589, 292590, 292591, 292592, 292593, 292594, 292595, 292596) c6( ) c7(gh, T, aC, fG, bO, bB, bS, ct, ci, eA, ad, av, jz, aT, h, K, an, H, he, ae, xa, af, CB, jf, Uf, B, aW, Y, auM, eH, g, cl, Z, eF, cm, bz, ek, hq, ck, ac, c, bE, wO, acS, apl, yP, U, hL, lF, dX, alv, EY, X, L); #c1(PAX1) c2(NP_006183) c3(paired box protein Pax-1 isoform 1) c4(162671) c5(162672, 162673, 162674, 162675, 162676, 162677) c6( ) c7(bcX, azs, bAV, cnX, Ln, bAZ, lq, lp, if, ff, bQ, en, qN, bfU, Ex); #c1(PAX2) c2(NP_000269) c3(paired box protein Pax-2 isoform b) c4( ) c5(6048, 6049, 6050, 6051, 6052) c6( ) c7(gh, T, ul, Uc, eR, cnZ, zX, aMq, bB, fr, HS, aoM, ci, aY, cnY, iJ, pn, ad, er, dP, py, MS, yQ, wG, tL, tT, aT, lG, dL, Nj, ae, af, eL, Ur, xQ, auY, s, aW, Y, agF, yq, Lc, zC, g, cl, t, dr, ck, sk, aug, uP, kn, es, vW, iA, bE, AM, FB, wB, hL, fP, dy, VB); #c1(PAX2) c2(NP_001291498) c3 (paired box protein Pax-2 isoform f) c4(116295) c5(116296, 116297) c6( ) c7(gh, T, ul, Uc, eR, cnZ, zX, aMq, bB, fr, HS, aoM, ci, aY, cnY, iJ, pn, ad, er, dP, py, MS, yQ, wG, tL, tT, aT, lG, dL, Nj, ae, af, eL, Ur, xQ, auY, s, aW, Y, agF, yq, Lc, zC, g, cl, t, dr, ck, sk, aug, uP, kn, es, vW, iA, bE, AM, FB, wB, hL, fP, dy, VB); #c1(PAX2) c2(NP_003978) c3(paired box protein Pax-2 isoform a) c4(142602, 142603) c5(142604, 142605, 142606, 142607, 142608, 142609, 142610, 142611, 142612, 142613, 142614, 142615) c6( ) c7(gh, T, ul, Uc, eR, cnZ, zX, aMq, bB, fr, HS, aoM, ci, aY, cnY, iJ, pn, ad, er, dP, py, MS, yQ, wG, tl, tT, aT, lG, dL, Nj, ae, af, eL, Ur, xQ, auY, s, aW, Y, agF, yq, Lc, zC, g, cl, t, dr, ck, sk, aug, uP, kn, es, vW, iA, bE, AM, FB, wB, hL, fP, dy, VB); #c1(PAX2) c2(NP_003979) c3(paired box protein Pax-2 isoform c) c4(142616, 142617) c5(142618, 142619, 142620, 142621, 142622, 142623, 142624, 142625, 142626, 142627, 142628, 142629) c6( ) c7(gh, T, ul, Uc, eR, cnZ, zX, aMq, bB, fr, HS, aoM, ci, aY, cnY, iJ, pn, ad, er, dP, py, MS, yQ, wG, tL, tT, aT, lG, dL, Nj, ae, af, eL, Ur, xQ, auY, s, aW, Y, agF, yq, Lc, zC, g, cl, t, dr, ck, sk, aug, uP, kn, es, vW, iA, bE, AM, FB, wB, hL, fP, dy, VB); #c1(PAX2) c2(NP_003981) c3 (paired box protein Pax-2 isoform e) c4(142630) c5(142631, 142632, 142633, 142634, 142635, 142636) c6( ) c7(gh, T, ul, Uc, eR, cnZ, zX, aMq, bB, fr, HS, aoM, ci, aY, cnY, iJ, pn, ad, er, dP, py, MS, yQ, wG, tL, tT, aT, lG, dL, Nj, ae, af, eL, Ur, xQ, auY, s, aW, Y, agF, yq, Lc, zC, g, cl, t, dr, ck, sk, aug, uP, kn, es, vW, iA, bE, AM, FB, wB, hL, fP, dy, VB); #c1(PAX3) c2(NP_000429) c3(paired box protein Pax-3 isoform PAX3a) c4(8887) c5(8888, 8889, 8890, 8891, 8892, 8893, 8894) c6( ) c7(ng, bM, T, dA, ha, aOH, jh, cob, ko, aln, bjN, coa, nA, eA, Na, bv, ft, amJ, Eo, Nn, KN, Ou, Nc, bqS, K, fb, ahE, dx, Gc, jf, lR, kj, Qr, are, Y, AQ, Az, ahl, aTa, eF, cm, bz, XT, bN, ck, Dy, lp, hk, mG, wk, Xu, aps, aPG, ps); #c1(PAX3) c2(NP_001120838) c3(paired box protein Pax-3 isoform PAX3i) c4(44818) c5(44819, 44820, 44821, 44822, 44823, 44824) c6( ) c7(ng, bM, T, dA, ha, aOH, jh, cob, ko, aln, bjN, coa, nA, eA, Na, bv, ft, amJ, Eo, Nn, KN, Ou, Nc, bqS, K, fb, ahE, dx, Gc, jf, lR, kj, Or, are, Y, AQ, Az, ahl, aTa, eF, cm, bz, XT, bN, ck, Dy, lp, hk, mG, wk, Xu, aps, aPG, ps); #c1(PAX3) c2(NP_039230) c3(paired box protein Pax-3 isoform PAX3b) c4(176909) c5(176910, 176911, 176912, 176913, 176914, 176915) c6( ) c7(ng, bM, T, dA, ha, aOH, jh, cob, ko, aln, bjN, coa, nA, eA, Na, bv, ft, amJ, Eo, Nn, KN, Ou, Nc, bqS, K, fb, ahE, dx, Gc, jf, lR, kj, Or, are, Y, AQ, Az, ahl, aTa, eF, cm, bz, XT, bN, ck, Dy, lp, hk, mG, wk, Xu, aps, aPG, ps); #c1(PAX3) c2(NP_852122) c3(paired box protein Pax-3 isoform PAX3) c4(254866) c5(254867, 254868, 254869, 254870, 254871, 254872) c6( ) c7(ng, bM, T, dA, ha, aOH, jh, cob, ko, aln, bjN, coa, nA, eA, Na, bv, ft, amJ, Eo, Nn, KN, Ou, Nc, bqS, K, fb, ahE, dx, Gc, jf, lR, kj, Qr, are, Y, AQ, Az, ahl, aTa, eF, cm, bz, XT, bN, ck, Dy, lp, hk, mG, wk, Xu, aps, aPG, ps); #c1(PAX3) c2(NP_852123) c3(paired box protein Pax-3 isoform PAX3d) c4(254873) c5( ) c6( ) c7(ng, bM, T, dA, ha, aOH, jh, cob, ko, aln, bjN, coa, nA, eA, Na, bv, ft, amJ, Eo, Nn, KN, Ou, Nc, bqS, K, fb, ahE, dx, Gc, jf, lR, kj, Qr, are, Y, AQ, Az, ahl, aTa, eF, cm, bz, XT, bN, ck, Dy, lp, hk, mG, wk, Xu, aps, aPG, ps); #c1(PAX3) c2(NP_852124) c3(paired box protein Pax-3 isoform PAX3e) c4(254874) c5(254875, 254876, 254877, 254878, 254879, 254880, 254881, 254882, 254883, 254884, 254885, 254886) c6( ) c7(ng, bM, T, dA, ha, aOH, jh, cob, ko, aln, bjN, coa, nA, eA, Na, bv, ft, amJ, Eo, Nn, KN, Ou, Nc, bqS, K, fb, ahE, dx, Gc, jf, lR, kj, Qr, are, Y, AQ, Az, ahl, aTa, eF, cm, bz, XT, bN, ck, Dy, lp, hk, mG, wk, Xu, aps, aPG, ps); #c1(PAX3) c2(NP_852125) c3(paired box protein Pax-3 isoform PAX3h) c4(254887) c5(254888, 254889, 254890, 254891, 254892, 254893) c6( ) c7(ng, bM, T, dA, ha, aOH, jh, cob, ko, aln, bjN, coa, nA, eA, Na, bv, ft, amJ, Eo, Nn, KN, Ou, Nc, bqS, K, fb, ahE, dx, Gc, jf, lR, kj, Qr, are, Y, AQ, Az, ahl, aTa, eF, cm, bz, XT, bN, ck, Dy, lp, hk, mG, wk, Xu, aps, aPG, ps); #c1(PAX3) c2(NP_852126) c3(paired box protein Pax-3 isoform PAX3g) c4(254894) c5(254895, 254896, 254897, 254898, 254899, 254900) c6( ) c7(ng, bM, T, dA, ha, aOH, jh, cob, ko, aLn, bjN, coa, nA, eA, Na, bv, ft, amJ, Eo, Nn, KN, Ou, Nc, bqS, K, fb, ahE, dx, Gc, jf, lR, kj, Qr, are, Y, AQ, Az, ahl, aTa, eF, cm, bz, XT, bN, ck, Dy, lp, hk, mG, wk, Xu, aps, aPG, ps); #c1(PAX4) c2(NP_006184) c3(paired box protein Pax-4) c4(162678) c5(162679, 162680, 162681, 162682, 162683, 162684) c6( ) c7(bL, A, mh, eR, coc, Y, Nn, TF, aj, dG, k, ei, J); #c1(PAX4) c2(XP_011514578) c3(paired box protein Pax-4 isoform X1) c4(307286, 307287) c5(307288, 307289, 307290, 307291, 307292, 307293, 307294, 307295, 307296, 307297, 307298, 307299, 307300) c6( ) c7(bL, A, mh, eR, coc, Y, Nn, TF, aj, dG, k, ei, J); #c1(PAX5) c2(NP_001267476) c3 (paired box protein Pax-5 isoform 2) c4(99599) c5(99600, 99601, 99602, 99603, 99604, 99605) c6( ) c7(oO, bM, T, Fv, aaH, if, aT, fd, amj, Jd, lN, ci, Tz, x, Vp, bv, Gt, aE, OG, fm, pX, jk, rB, K, bU, pk, eL, hm, xQ, bPf, KS, aW, hC, Az, Qq, aVu, Ku, g, as, eF, ou, bb, eM, nB, coi, iT, vy, cj, Zn, k, m, Xu, Vj, asW, dt, QO, aEj, aYZ, aw, n, aj, fr, ct, qN, eW, b, ft, i, coe, adX, h, eV, hn, dL, aDC, pi, dx, Yl, mR, AQ, fe, aiL, aq, yq, Og, mo, dw, Wt, hq, amt, kT, bP, cA, adZ, dB, Vy, Be, eo, eQ, YH, anx, B, bL, fM, aGt, jx, eX, aA, cz, zN, cog, v, j, ajM, V, avD, q, er, eA, XX, Rl, hW, nv, aWJ, aT, rO, dz, adV, an, ae, af, jf, ak, PK, s, iU, oV, Y, ate, Pg, r, en, hf, cl, t, AA, apx, pb, ik, dS, ck, nM, ac, iM, ll, nG, hO, wq, cdM, bt, ava, id, iW, ha, jh, bS, hF, iJ, ad, afb, wD, Jm, dP, av, hd, iC, Db, acu, zK, ds, qh, oW, bby, fb, Yi, mu, d, je, et, ff, xs, Of, eZ, aFr, aF, hp, iV, Z, bz, qa, cgE, eK, gk, axs, coh, cof, aVv, gZ, awk, bC, ei, pk, y, xg, atd, cod); #c1(PAX5) c2(NP_001267477) c3(paired box protein Pax-5 isoform 3) c4(99606) c5(99607, 99608, 99609, 99610, 99611, 99612) c6( ) c7(oO, bM, T, Fv, aaH, if, aT, fd, amj, Jd, lN, ci, Tz, x, Vp, bv, Gt, aE, OG, fm, pX, jk, rB, K, bU, pk, eL, hm, xQ, bPf, kS, aW, hC, Az, Qq, aVu, Ku, g, as, eF, ou, bb, eM, nB, coi, iT, vy, cj, Zn, k, m, Xu, Vj, asW, dt, QO, aEj, aYZ, aw, n, aj, fr, ct, qN, eW, b, ft, i, coe, adX, h, eV, hn, dL, aDC, pi, dx, Yl, mR, AQ, fe, aiJ, aq, yq, Og, mo, dw, Wt, hq, amt, KT, bP, cA, adZ, dB, Vy, Be, eo, eQ, YH, anx, B, bL, fM, aGt, jx, eX, aA, cz, zN, cog, v, j, ajM, V, avD, q, er, eA, XX, Rl, hW, nv, aWJ, aT, rO, dz, adV, an, ae, af, jf, ak, PK, s, iU, oV, Y, ate, Pg, r, en, hf, cl, t, AA, apx, pb, iK, dS, ck, nM, ac, iM, ll, nG, hO, wq, cdM, bt, ava, id, iW, ha, jh, bS, hF, iJ, ad, afb, wD, Jm, dP, av, hd, iC, Db, acu, zK, ds, qh, oW, bby, fb, Yi, mu, d, je, et, ff, xs, Of, eZ, aFr, aF, hp, iV, Z, bz, qa, cgE, eK, gk, axs, coh, cof, aVv, gZ, awk, bC, ei, pK, y, xg, atd, cod); #c1(PAX5) c2(NP_001267478) c3(paired box protein Pax-5 isoform 4) c4( ) c5(99613) c6( ) c7(oO, bM, T, Fv, aaH, if, al, fd, amj, Jd, lN, ci, Tz, x, Vp, bv, Gt, aE, oG, fm, pX, jk, rB, K, bU, pk, eL, hm, xQ, bPf, kS, aW, hC, Az, Qq, aVu, Ku, g, as, ef, ou, bb, eM, nB, coi, iT, vy, cj, Zn, k, m, Xu, Vj, asW, dt, QO, aEj, aYZ, aw, n, aj, fr, ct, qN, eW, b, ft, i, coe, adX, h, eV, hn, dL, aDC, pi, dx, Yl, mR, AQ, fe, aiL, aq, yq, Og, mo, dw, Wt, hq, amt, kT, bP, cA, adZ, dB, Vy, Be, eo, eQ, YH, anx, B, bL, fM, aGt, jx, eX, aA, cz, zN, cog, v, j, ajM, V, avD, q, er, eA, XX, Rl, hW, nv, aW, J, aT, rO, dz, adV, an, ae, af, jf, ak, PK, s, iU, oV, Y, ate, Pg, r, en, hf, cl, t, AA, apx, pb, ik, dS, ck, nM, ac, iM, ll, nG, hO, wq, cdM, bt, ava, id, iW, ha, jh, bS, hF, iJ, ad, afb, wD, Jm, dP, av, hd, iC, Db, acu, zK, ds, qh, oW, bby, fb, Yi, mu, d, je, et, ff, xs, Of, eZ, aFr, af, hp, iV, Z, bz, qa, cgE, ek, gk, axs, coh, cof, aVv, gZ, awk, bC, ei, pK, y, xg, atd, cod); #c1(PAX5) c2(NP_001267479) c3(paired box protein Pax-5 isoform 5) c4( ) c5(99614, 99615, 99616, 99617, 99618) c6( ) c7(oO, bM, T, Fv, aaH, if, al, fd, amj, Jd, lN, ci, Tz, x, Vp, bv, Gt, aE, OG, fm, pX, jk, rB, K, bU, pk, eL, hm, xQ, bPf, KS, aW, hC, Az, Qq, aVu, Ku, g, as, eF, ou, bb, eM, nB, coi, iT, vy, cj, Zn, k, m, Xu, Vj, asW, dt, QO, aEj, aYZ, aw, n, aj, fr, ct, qN, eW, b, ft, i, coe, adX, h, eV, hn, dL, aDC, pi, dx, Yl, mR, AQ, fe, aiJ, aq, yq, Og, mo, dw, Wt, hq, amt, kT, bP, cA, adZ, dB, Vy, Be, eo, eQ, YH, anx, B, bL, fM, aGt, jx, eX, aA, cz, zN, cog, v, j, ajM, V, avD, q, er, eA, XX, Rl, hW, nv, aWJ, aT, rO, dz, adV, an, ae, af, jf, ak, PK, s, iU, oV, Y, ate, Pg, r, en, hf, cl, t, AA, apx, pb, ik, dS, ck, nM, ac, iM, ll, nG, hO, wq, cdM, bt, ava, id, iW, ha, jh, bS, hF, iJ, ad, afb, wD, Jm, dP, av, hd, iC, Db, acu, zK, ds, qh, oW, bby, fb, Yi, mu, d, je, et, ff, xs, Of, eZ, aFr, aF, hp, iV, Z, bz, qa, cgE, ek, gk, axs, coh, cof, aVv, gZ, awk, bC, ei, pK, y, xg, atd, cod); #c1(PAX5) c2(NP_001267481) c3(paired box protein Pax-5 isoform 7) c4(99619, 99620) c5(99621, 99622, 99623, 99624, 99625, 99626) c6( ) c7(oO, bM, T, Fv, aaH, if, al, fd, amj, Jd, lN, ci, Tz, x, Vp, bv, Gt, aE, OG, fm, pX, jk, rB, K, bU, pk, eL, hm, xQ, bPf, kS, aW, hC, Az, Qq, aVu, Ku, g, as, eF, ou, bb, eM, nB, coi, iT, vy, cj, Zn, k, m, Xu, Vj, asW, dt, QO, aEj, aYZ, aw, n, aj, fr, ct, qN, eW, b, ft, i, coe, adX, h, eV, hn, dL, aDC, pi, dx, Yl, mR, AQ, fe, aiL, aq, yq, Og, mo, dw, Wt, hq, amt, kT, bP, cA, adZ, dB, Vy, Be, eo, eQ, YH, anx, B, bL, fM, aGt, jx, eX, aA, cz, zN, cog, v, j, ajM, V, avD, q, er, eA, XX, Rl, hW, nv, aWJ, aT, rO, dz, adV, an, ae, af, jf, ak, PK, s, iU, oV, Y, ate, Pg, r, en, hf, cl, t, AA, apx, pb, ik, dS, ck, nM, ac, iM, ll, nG, hO, wq, cdM, bt, ava, id, iW, ha, jh, bS, hF, iJ, ad, afb, wD, Jm, dP, av, hd, iC, Db, acu, zK, ds, qh, oW, bby, fb, Yi, mu, d, je, et, ff, xs, Of, eZ, aFr, aF, hp, iV, Z, bz, qa, cgE, ek, gk, axs, coh, cof, aVv, gZ, awk, bC, ei, pK, y, xg, atd, cod); #c1(PAX5) c2(NP_001267482) c3(paired box protein Pax-5 isoform 8) c4(99627) c5(99628, 99629, 99630, 99631, 99632, 99633) c6( ) c7(oO, bM, T, Fv, aaH, if, al, fd, amj, Jd, lN, ci, Tz, x, Vp, bv, Gt, aE, OG, fm, pX, jk, rB, K, bU, pk, eL, hm, xQ, bPf, kS, aW, hC, Az, Qq, aVu, Ku, g, as, eF, ou, bb, eM, nB, coi, iT, vy, cj, Zn, k, m, Xu, Vj, asW, dt, QO, aEj, aYZ, aw, n, aj, fr, ct, qN, eW, b, ft, i, coe, adX, h, eV, hn, dL, aDC, pi, dx, Yl, mR, AQ, fe, aiL, aq, yq, Og, mo, dw, Wt, hq, amt, KT, bP, cA, adZ, dB, Vy, Be, eo, eQ, YH, anx, B, bL, fM, aGt, jx, eX, aA, cz, zN, cog, v, j, ajM, V, avD, q, er, eA, XX, Rl, hW, nv, aWJ, aT, rO, dz, adV, an, ae, af, jf, ak, PK, s, iU, oV, Y, ate, Pg, r, en, hf, cl, t, AA, apx, pb, ik, dS, ck, nM, ac, iM, ll, nG, hO, wq, cdM, bt, ava, id, iW, ha, jh, bS, hF, iJ, ad, afb, wD, Jm, dP, av, hd, iC, Db, acu, zK, ds, qh, oW, bby, fb, Yi, mu, d, je, et, ff, xs, Of, eZ, aFr, aF, hp, iV, Z, bz, qa, cgE, ek, gk, axs, coh, cof, aVv, gZ, awk, bC, ei, pK, y, xg, atd, cod); #c1(PAX5) c2(NP_001267483) c3(paired box protein Pax-5 isoform 9) c4(99634) c5(99635, 99636, 99637, 99638, 99639, 99640) c6( ) c7(oO, bM, T, Fv, aaH, if, al, fd, amj, Jd, lN, ci, Tz, x, Vp, bv, Gt, aE, OG, fm, pX, jk, rB, K, bU, pk, eL, hm, xQ, bPf, kS, aW, hC, Az, Qq, aVu, Ku, g, as, eF, ou, bb, eM, nB, coi, iT, vy, cj, Zn, k, m, Xu, Vj, asW, dt, QO, aEj, aYZ, aw, n, aj, fr, ct, qN, eW, b, ft, i, coe, adX, h, eV, hn, dL, aDC, pi, dx, Yl, mR, AQ, fe, aiL, aq, yq, Og, mo, dw, Wt, hq, amt, kT, bP, cA, adZ, dB, Vy, Be, eo, eQ, YH, anx, B, bL, fM, aGt, jx, eX, aA, cz, zN, cog, v, j, ajM, V, avD, q, er, eA, XX, Rl, hW, nv, aWJ, aT, rO, dz, adV, an, ae, af, jf, ak, PK, s, iU, oV, Y, ate, Pg, r, en, hf, cl, t, AA, apx, pb, ik, dS, ck, nM, ac, iM, ll, nG, hO, wq, cdM, bt, ava, id, iW, ha, jh, bS, hF, iJ, ad, afb, wD, Jm, dP, av, hd, iC, Db, acu, zK, ds, qh, oW, bby, fb, Yi, mu, d, je, et, ff, xs, Of, eZ, aFr, aF, hp, iV, Z, bz, qa, cgE, ek, gk, axs, coh, cof, aVv, gZ, awk, bC, ei, pk, y, xg, atd, cod); #c1(PAX5) c2(NP_001267484) c3(paired box protein Pax-5 isoform 10) c4(99641) c5(99642, 99643, 99644, 99645, 99646, 99647) c6( ) c7(oO, bM, T, Fv, aaH, if, al, fd, amj, Jd, lN, ci, Tz, x, Vp, bv, Gt, aE, oG, fm, pX, jk, rB, K, bU, pk, eL, hm, xQ, bPf, kS, aW, hC, Az, Qq, aVu, Ku, g, as, eF, ou, bb, eM, nB, coi, iT, vy, cj, Zn, k, m, Xu, Vj, asW, dt, QO, aEj, aYZ, aw, n, aj, fr, ct, qN, eW, b, ft, i, coe, adX, h, eV, hn, dL, aDC, pi, dx, Yl, mR, AQ, fe, aiL, aq, yq, Og, mo, dw, Wt, hq, amt, KT, bP, cA, adZ, dB, Vy, Be, eo, eQ, YH, anx, B, bL, fM, aGt, jx, eX, aA, cz, zN, cog, v, j, ajM, V, avD, q, er, eA, XX, Rl, hW, nv, aWJ, aT, r, dz, adV, an, ae, af, jf, ak, PK, s, iU, oV, Y, ate, Pg, r, en, hf, cl, t, AA, apx, pb, ik, dS, ck, nM, ac, iM, ll, nG, hO, wq, cdM, bt, ava, id, iW, ha, jh, bS, hF, iJ, ad, afb, wD, Jm, dP, av, hd, iC, Db, acu, zK, ds, qh, oW, bby, fb, Yi, mu, d, je, et, ff, xs, Of, eZ, aFr, aF, hp, iV, Z, bz, qa, cgE, ek, gk, axs, coh, cof, aVv, gZ, awK, bC, ei, pK, y, xg, atd, cod); #c1(PAX5) c2(NP_001267485) c3(paired box protein Pax-5 isoform (1) c4( ) c5(99648, 99649, 99650, 99651, 99652) c6( ) c7(oO, bM, T, Fv, aaH, if, al, fd, amj, Jd, lN, ci, Tz, x, Vp, bv, Gt, aE, OG, fm, pX, jk, rB, K, bU, pk, eL, hm, xQ, bPf, kS, aW, hC, Az, Qq, aVu, Ku, g, as, eF, ou, bb, eM, nB, coi, iT, vy, cj, Zn, k, m, Xu, Vj, asW, dt, QO, aEj, aYZ, aw, n, aj, fr, ct, qN, eW, b, ft, i, coe, adX, h, eV, hn, dL, aDC, pi, dx, Yl, mR, AQ, fe, aiL, aq, yq, Og, mo, dw, Wt, hq, amt, kT, bP, cA, adZ, dB, Vy, Be, eo, eQ, YH, anx, B, bL, fM, aGt, jx, eX, aA, cz, zN, cog, v, j, ajM, V, avD, q, er, eA, XX, Rl, hW, nv, aWJ, aT, rO, dz, adV, an, ae, af, jf, ak, PK, s, iU, oV, Y, ate, Pg, r, en, hf, cl, t, AA, apx, pb, ik, dS, ck, nM, ac, iM, ll, nG, hO, wq, cdM, bt, ava, id, iW, ha, jh, bS, hF, iJ, ad, afb, wD, Jm, dP, av, hd, iC, Db, acu, zK, ds, qh, oW, bby, fb, Yi, mu, d, je, et, ff, xs, Of, eZ, aFr, aF, hp, iV, Z, bz, qa, cgE, ek, gk, axs, coh, cof, aVv, gZ, awK, bC, ei, pK, y, xg, atd, cod); #c1(PAX5) c2(NP_057953) c3(paired box protein Pax-5 isoform 1) c4(192635, 192636) c5(192637, 192638, 192639, 192640, 192641, 192642, 192643, 192644, 192645) c6( ) c7(oO, bM, T, Fv, aaH, if, al, fd, amj, Jd, lN, ci, Tz, x, Vp, bv, Gt, aE, oG, fm, pX, jk, rB, K, bU, pk, eL, hm, xQ, bPf, KS, aW, hC, Az, Qq, aVu, Ku, g, as, eF, ou, bb, eM, nB, coi, iT, vy, cj, Zn, k, m, Xu, Vj, asW, dt, QO, aEj, aYZ, aw, n, aj, fr, ct, qN, eW, b, ft, i, coe, adX, h, eV, hn, dL, aDC, pi, dx, Yl, mR, AQ, fe, aiL, aq, yq, Og, mo, dw, Wt, hq, amt, KT, bP, cA, adZ, dB, Vy, Be, eo, eQ, YH, anx, B, bL, fM, aGt, jx, eX, aA, cz, zN, cog, v, j, ajM, V, avD, q, er, eA, XX, Rl, hW, nv, aWJ, aT, rO, dz, adV, an, ae, af, jf, ak, PK, s, iU, oV, Y, ate, Pg, r, en, hf, cl, t, AA, apx, pb, ik, dS, ck, nM, ac, iM, ll, nG, hO, wq, cdM, bt, ava, id, iW, ha, jh, bS, hF, iJ, ad, afb, wD, Jm, dP, av, hd, iC, Db, acu, zK, ds, qh, oW, bby, fb, Yi, mu, d, je, et, ff, xs, Of, eZ, aFr, aF, hp, iV, Z, bz, qa, cgE, ek, gk, axs, coh, cof, aVv, gZ, awk, bC, ei, pK, y, xg, atd, cod); #c1(PAX5) c2(XP_005251538) c3(paired box protein Pax-5 isoform X3) c4( ) c5(270998) c6( ) c7(oO, bM, T, Fv, aaH, if, al, fd, amj, Jd, lN, ci, Tz, x, Vp, bv, Gt, aE, oG, fm, pX, jk, rB, K, bU, pk, eL, hm, xQ, bPf, KS, aW, hC, Az, Qq, aVu, Ku, g, as, eF, ou, bb, eM, nB, coi, iT, vy, cj, Zn, k, m, Xu, Vj, asW, dt, QO, aEj, aYZ, aw, n, aj, fr, ct, qN, eW, b, ft, i, coe, adX, h, eV, hn, dL, aDC, pi, dx, Yl, mR, AQ, fe, aiL, aq, yq, Og, mo, dw, Wt, hq, amt, kT, bP, cA, adZ, dB, Vy, Be, eo, eQ, YH, anx, B, bL, fM, aGt, jx, eX, aA, cz, zN, cog, v, j, ajM, V, avD, q, er, eA, XX, Rl, hW, nv, aWJ, aT, rO, dz, adV, an, ae, af, jf, ak, PK, s, iU, oV, Y, ate, Pg, r, en, hf, cl, t, AA, apx, pb, ik, dS, ck, nM, ac, iM, ll, nG, hO, wq, cdM, bt, ava, id, iW, ha, jh, bS, hF, iJ, ad, afb, wD, Jm, dP, av, hd, iC, Db, acu, zK, ds, qh, oW, bby, fb, Yi, mu, d, je, et, ff, xs, Of, eZ, aFr, aF, hp, iV, Z, bz, qa, cgE, ek, gk, axs, coh, cof, aVv, gZ, awK, bC, ei, pK, y, xg, atd, cod); #c1(PAX5) c2(XP_011516198) c3(paired box protein Pax-5 isoform X1) c4(309422, 309423) c5(309424, 309425, 309426, 309427, 309428, 309429, 309430) c6( ) c7(oO, bM, T, Fv, aaH, if, al, fd, amj, Jd, lN, ci, Tz, x, Vp, bv, Gt, aE, oG, fm, pX, jk, rB, K, bU, pk, eL, hm, xQ, bPf, KS, aW, hC, Az, Qq, aVu, Ku, g, as, eF, ou, bb, eM, nB, coi, iT, vy, cj, Zn, k, m, Xu, Vj, asW, dt, QO, aEj, aYZ, aw, n, aj, fr, ct, qN, eW, b, ft, i, coe, adX, h, eV, hn, dL, aDC, pi, dx, Yl, mR, AQ, fe, aiL, aq, yq, Og, mo, dw, Wt, hq, amt, kT, bP, cA, adZ, dB, Vy, Be, eo, eQ, YH, anx, B, bL, fM, aGt, jx, eX, aA, cz, zN, cog, v, j, ajM, V, avD, q, er, eA, XX, Rl, hW, nv, aWJ, aT, rO, dz, adV, an, ae, af, jf, ak, PK, s, iU, oV, Y, ate, Pg, r, en, hf, cl, t, AA, apx, pb, ik, dS, ck, nM, ac, iM, ll, nG, hO, wq, cdM, bt, ava, id, iW, ha, jh, bS, hF, iJ, ad, afb, wD, Jm, dP, av, hd, iC, Db, acu, zK, ds, qh, oW, bby, fb, Yi, mu, d, je, et, ff, xs, Of, eZ, aFr, aF, hp, iV, Z, bz, qa, cgE, ek, gk, axs, coh, cof, aVv, gZ, awk, bC, ei, pk, y, xg, atd, cod); #c1(PAX5) c2(XP_011516199) c3(paired box protein Pax-5 isoform X2) c4( ) c5(309431, 309432, 309433, 309434, 309435) c6( ) c7(oO, bM, T, Fv, aaH, if, al, fd, amj, Jd, lN, ci, Tz, x, Vp, bv, Gt, aE, oG, fm, pX, jk, rB, K, bU, pk, eL, hm, xQ, bPf, kS, aW, hC, Az, Qq, aVu, Ku, g, as, eF, ou, bb, eM, nB, coi, iT, vy, cj, Zn, k, m, Xu, Vj, asW, dt, QO, aEj, aYZ, aw, n, aj, fr, ct, qN, eW, b, ft, i, coe, adX, h, eV, hn, dL, aDC, pi, dx, Yl, mR, AQ, fe, aiL, aq, yq, Dg, mo, dw, Wt, hq, amt, kT, bP, cA, adZ, dB, Vy, Be, eo, eQ, YH, anx, B, bL, fM, aGt, jx, eX, aA, cz, zN, cog, v, j, ajM, V, avD, q, er, eA, XX, Rl, hW, nv, aWJ, aT, rO, dz, adV, an, ae, af, jf, ak, PK, s, iU, oV, Y, ate, Pg, r, en, hf, cl, t, AA, apx, pb, ik, dS, ck, nM, ac, iM, ll, nG, hO, wq, cdM, bt, ava, id, iW, ha, jh, bS, hF, iJ, ad, afb, wD, Jm, dP, av, hd, iC, Db, acu, zK, ds, qh, oW, bby, fb, Yi, mu, d, je, et, ff, xs, Of, eZ, aFr, aF, hp, iV, Z, bz, qa, cgE, ek, gk, axs, coh, cof, aVv, gZ, awk, bC, ei, pk, y, xg, atd, cod); #c1(PAX6) c2(NP_001245391) c3(paired box protein Pax-6 isoform b) c4(93443, 93444) c5(93445, 93446, 93447, 93448, 93449, 93450, 93451, 93452, 93453, 93454, 93455, 93456, 93457, 93458, 93459, 93460, 93461, 93462) c6( ) c7(T, os, asg, acz, cop, zh, ci, Na, cfq, aE, arm, aEJ, bki, K, eL, aGb, col, aW, Az, OZ, vs, as, eM, cok, coo, nw, coq, asW, dD, cnZ, fN, n, aj, ko, nA, Dr, aqK, dL, vd, he, bbG, kj, bFc, mo, QK, fq, kn, KE, bP, cA, Be, el, JJ, bL, nx, aA, cz, v, q, J, er, eA, anA, aT, Nc, NG, anz, bq, cot, aQu, ae, jf, le, oV, Y, la, r, kC, bN, bMZ, mf, Bd, com, ha, ml, rl, cor, oZ, eq, cos, qi, tK, ad, coj, con, d, lR, rM, aF, aTa, bz, dr, bF, Hw, Ew, ajw, ei, oQ); #c1 (PAX6) c2(NP_001245394) c3(paired box protein Pax-6 isoform a) c4(93463, 93464, 93465) c5(93466, 93467, 93468, 93469, 93470, 93471, 93472, 93473, 93474, 93475, 93476, 93477, 93478, 93479, 93480, 93481, 93482, 93483, 93484, 93485, 93486, 93487, 93488, 93489, 93490, 93491, 93492, 93493, 93494) c6( ) c7(T, os, asg, acz, cop, zh, ci, Na, cfq, aE, arm, aEJ, bki, K, eL, aGb, col, aW, Az, OZ, vs, as, eM, cok, coo, nw, coq, asW, dD, cnZ, fN, n, aj, ko, nA, Dr, aqK, dL, vd, he, bbG, kj, bFc, mo, QK, fq, kn, KE, bP, cA, Be, el, JJ, bL, nx, aA, cz, v, q, J, er, eA, anA, aT, Nc, NG, anz, bq, cot, aQu, ae, jf, le, oV, Y, la, r, kC, bN, bMZ, mf, Bd, com, ha, ml, rl, cor, oZ, eq, cos, qi, tK, ad, coj, con, d, lR, rM, aF, aTa, bz, dr, bF, Hw, Ew, ajw, ei, oQ); #c1(PAX6) c2(XP_006718309) c3(paired box protein Pax-6 isoform X8) c4( ) c5(291699, 291700, 291701, 29/702, 291703, 291704) c6( ) c7(T, os, asg, acz, cop, zh, ci, Na, cfq, aE, arm, aEJ, bki, K, eL, aGb, col, aW, Az, OZ, vs, as, eM, cok, coo, nw, coq, asW, dD, cnZ, fN, n, aj, ko, nA, Dr, aqK, dl, vd, he, bbG, kj, bFc, mo, QK, fq, kn, KE, bP, cA, Be, el, JJ, bL, nx, aA, cz, v, q, J, er, eA, anA, aT, Nc, NG, anz, bq, cot, aQu, ae, jf, le, oV, Y, la, r, kC, bN, bMZ, mf, Bd, com, ha, ml, rl, cor, oZ, eq, cos, qi, tK, ad, coj, con, d, lR, rM, aF, aTa, bz, dr, bF, Hw, Ew, ajw, ei, oQ); #c1(PAX6) c2(XP_011518454) c3(paired box protein Pax-6 isoform X7) c4(312511, 312512) c5(312513, 312514, 312515, 312516, 312517, 312518, 312519, 312520, 312521, 312522, 312523, 312524) c6( ) c7(T, os, asg, acz, cop, zh, ci, Na, cfq, aE, arm, aEJ, bki, K, eL, aGb, col, aW, Az, OZ, vs, as, eM, cok, coo, nw, coq, asW, dD, cnZ, fN, n, aj, ko, nA, Dr, aqK, dL, vd, he, bbG, kj, bFc, mo, QK, fq, kn, KE, bP, cA, Be, el, JJ, bL, nx, aA, cz, v, q, J, er, eA, anA, aT, Nc, NG, anz, bq, cot, aQu, ae, jf, le, oV, Y, la, r, kC, bN, bMZ, mf, Bd, com, ha, ml, rl, cor, oZ, eq, cos, qi, tk, ad, coj, con, d, lR, rM, aF, aTa, bz, dr, bF, Hw, Ew, ajw, ei, oQ); #c1(PAX8) c2(NP_003457) c3 (paired box protein Pax-8 isoform PAX8A) c4(138457) c5(138458, 138459, 138460, 138461, 138462) c6( ) c7(T, AS, n, hz, fr, ci, ml, aon, aYs, iJ, er, cou, dP, Vp, hd, Db, RN, pX, HD, Fx, AN, Xh, nJ, s, ff, agF, en, cl, g, zE, t, f, ck, sk, qJ, Qy, nM, eM, nB, hr, hL, m, gT, aMl, bwz); #c1(PAX8) c2(NP_039246) c3(paired box protein Pax-8 isoform PAX8C) c4(176916) c5(176917, 176918, 176919, 176920, 176921, 176922, 176923, 176924, 176925) c6( ) c7(T, AS, n, hz, fr, ci, ml, aon, aYs, iJ, er, cou, dP, Vp, hd, Db, RN, pX, HD, Fx, AN, Xh, nJ, s, ff, agF, en, cl, g, zE, t, f, ck, sk, qJ, Qy, nM, eM, nB, hr, hl, m, gT, aMl, bwz); #c1(PAX8) c2(NP_039247) c3 (paired box protein Pax-8 isoform PAX8D) c4(176926) c5(176927, 176928, 176929, 176930, 176931) c6( ) c7(T, AS, n, hz, fr, ci, ml, aon, aYs, iJ, er, cou, dP, Vp, hd, Db, RN, pX, hD, Fx, AN, Xh, nJ, s, ff, agF, en, cl, g, zE, t, f, ck, sk, qJ, Qy, nM, eM, nB, hr, hl, m, gT, aMl, bwz); #c1(PAX8) c2(NP_054698) c3(paired box protein Pax-8 isoform PAX8E) c4(177280) c5(177281, 177282, 177283, 177284, 177285) c6( ) c7(T, AS, n, hz, fr, ci, ml, aon, aYs, iJ, er, cou, dP, Vp, hd, Db, RN, pX, hD, Fx, AN, Xh, nJ, s, ff, agF, en, cl, g, zE, t, f, ck, sk, qJ, Qy, nM, eM, nB, hr, hl, m, gT, aMl, bwz); #c1(PAX8) c2(XP_011510092) c3(paired box protein Pax-8 isoform X1) c4( ) c5(300698, 300699) c6( ) c7(T, AS, n, hz, fr, ci, ml, aon, aYs, iJ, er, cou, dP, Vp, hd, Db, RN, pX, hD, Fx, AN, Xh, nJ, s, ff, agF, en, cl, g, zE, t, f, ck, sk, qJ, Qy, nM, eM, nB, hr, hl, m, gT, aMl, bwz); #c1(PAX8) c2(XP_011510094) c3(paired box protein Pax-8 isoform X3) c4( ) c5(300700) c6( ) c7(T, AS, n, hz, fr, ci, ml, aon, aYs, iJ, er, cou, dP, Vp, hd, Db, RN, pX, hD, Fx, AN, Xh, nJ, s, ff, agF, en, cl, g, zE, t, f, ck, sk, qJ, Qy, nM, eM, nB, hr, hL, m, gT, aMl, bwz); #c1(PAX8) c2(XP_011510095) c3(paired box protein Pax-8 isoform X4) c4( ) c5(300701, 300702) c6( ) c7(T, AS, n, hz, fr, ci, ml, aon, aYs, iJ, er, cou, dP, Vp, hd, Db, RN, pX, hD, Fx, AN, Xh, nJ, s, ff, agF, en, cl, g, zE, t, f, ck, sk, qJ, Qy, nM, eM, nB, hr, hL, m, gT, aMl, bwz); #c1(PAX9) c2(NP_006185) c3(paired box protein Pax-9) c4(162685, 162686) c5(162687, 162688, 162689, 162690, 162691, 162692, 162693, 162694, 162695, 162696, 162697, 162698, 162699, 162700, 162701, 162702, 162703) c6( ) c7(Na, bv, apM, bjy, nv, Y, bjB, Az, Nn, Nc, cov, aml, ckd, zo, amH); #c1(PAX1P1) c2(NP_031375) c3(PAX-interacting protein 1) c4(172046) c5( ) c6( ) c7(af); #c1(PAX1P1) c2(XP_005249596) c3(PAX-interacting protein 1 isoform X2) c4(269711) c5( ) c6( ) c7(af); #c1(PAX1P1) c2(XP_011514284) c3(PAX-interacting protein 1 isoform X1) c4(306871) c5( ) c6( ) c7(af); #c1(PAX1P1) c2(XP_011514286) c3(PAX-interacting protein 1 isoform X4) c4( ) c5(306872, 306873, 306874, 306875, 306876, 306877, 306878, 306879, 306880, 306881, 306882, 306883, 306884, 306885, 306886, 306887, 306888, 306889, 306890, 306891, 306892, 306893, 306894, 306895) c6( ) c7(af); #c1(PBK) c2(XP_006716431) c3(lymphokine-activated killer T-cell-originated protein kinase isoform X1) c4(290301) c5(290302, 290303, 290304, 290305, 290306, 290307) c6( ) c7(T, jh, bB, q, eW, bag, XX, av, jz, aT, kH, dL, ey, K, pk, eL, dx, kl, aW, Y, r, dN, alJ, bu, bz, bE, MC, B); #c1(PBLD) c2(NP_001028255) c3 (phenazine biosynthesis-like domain-containing protein isoform b) c4(30423) c5(30424, 30425, 30426, 30427, 30428, 30429) c6( ) c7(xS); #c1(PBLD) c2(NP_071412) c3(phenazine biosynthesis-like domain-containing protein isoform a) c4(211511, 211512, 211513) c5(211514, 211515, 211516, 211517, 211518, 211519, 211520, 211521, 211522, 211523, 211524, 211525, 211526, 211527, 211528, 211529) c6( ) c7(xS); #c1(PBLD) c2(XP_011538362) c3(phenazine biosynthesis-like domain-containing protein isoform X4) c4( ) c5(336050, 336051, 336052, 336053, 336054, 336055) c6( ) c7(xS); #c1(PBOV1) c2(NP_067648) c3(prostate and breast cancer overexpressed gene 1 protein) c4(209225) c5(209226, 209227, 209228, 209229, 209230, 209231) c6( ) c7(G, T, aW, eL, aT, dl, np, ae, eA, ad); #c1(PBRM1) c2(NP_060783) c3(protein polybromo-1) c4(197768, 197769, 197770, 197771) c5(197772, 197773, 197774, 197775, 197776, 197777, 197778, 197779, 197780, 197781, 197782, 197783, 197784, 197785, 197786, 197787) c6( ) c7(Mb, T, mS, n, fr, ci, pd, av, jz, py, aE, mW, ds, aT, bK, mY, mV, mU, aW, gl, bz, mZ, mX, es, cA, iA, go, RW, mT, VB, hS); #c1(PBRM1) c2(XP_005265332) c3(protein polybromo-1 isoform X4) c4( ) c5(279463, 279464) c6( ) c7(Mb, T, mS, n, fr, ci, pd, av, jz, py, aE, mW, ds, aT, bK, mY, mV, mU, aW, gl, bz, mZ, mX, es, cA, iA, go, RW, mT, VB, hS); #c1(PBRM1) c2(XP_011532205) c3 (protein polybromo-l isoform X1) c4( ) c5(328233, 328234, 328235, 328236, 328237, 328238, 328239, 328240, 328241, 328242, 328243, 328244, 328245, 328246, 328247, 328248, 328249, 328250, 328251, 328252, 328253, 328254, 328255, 328256) c6( ) c7(Mb, T, mS, n, fr, ci, pd, av, jz, py, aE, mW, ds, aT, bK, mY, mV, mU, aW, gl, bz, mZ, mX, es, CA, iA, go, RW, mT, VB, hS); #c1(PBRM1) c2(XP_011532208) c3(protein polybromo-1 isoform X5) c4(328257) c5(328258, 328259, 328260, 328261, 328262, 328263) c6( ) c7(Mb, T, mS, n, fr, ci, pd, av, jz, py, aE, mW, ds, aT, bK, mY, mV, mU, aW, gl, bz, mZ, mX, es, cA, iA, go, RW, mT, VB, hS); #c1(PBRM1) c2(XP_011532213) c3(protein polybromo-1 isoform X9) c4(328264) c5( ) c6( ) c7(Mb, T, mS, n, fr, ci, pd, av, jz, py, aE, mW, ds, aT, bK, mY, mV, mU, aW, gl, bz, mZ, mX, es, cA, iA, go, RW, mT, VB, hS); #c1(PBRM1) c2(XP_011532216) c3 (protein polybromo-1 isoform X12) c4(328265) c5(328266, 328267, 328268, 328269, 328270, 328271) c6( ) c7(Mb, T, mS, n, fr, ci, pd, av, jz, py, aE, mW, ds, aT, bK, mY, mV, mU, aW, gl, bz, mZ, mX, es, cA, iA, go, RW, mT, VB, hS); #c1(PBRM1) c2(XP_011532219) c3(protein polybromo-1 isoform X15) c4(328272, 328273) c5(328274, 328275, 328276, 328277, 328278, 328279) c6( ) c7(Mb, T, mS, n, fr, ci, pd, av, jz py, aE, mW, ds, aT, bK, mY, mV, mU, aW, gl, bz, mZ, mX, es, cA, iA, go, RW, mT, VB, hS); #c1(PBRM1) c2(XP_011532221) c3(protein polybromo-1 isoform X17) c4(328280, 328281) c5(328282, 328283, 328284, 328285, 328286, 328287, 328288, 328289, 328290, 328291) c6( ) c7(Mb, T, mS, n, fr, ci, pd, av, jz, py, aE, mW, ds, aT, bK, mY, mV, mU, aW, gl, bz, mZ, mX, es, cA, iA, go, RW, mT, VB, hS); #c1(PBX1) c2(NP_001191890) c3(pre-B-cell leukemia transcription factor 1 isoform 2) c4(81855, 81856) c5(81857, 81858, 81859, 81860, 81861, 81862, 81863, 81864, 81865, 81866, 81867, 81868, 81869, 81870, 81871, 81872, 81873, 81874, 81875, 81876, 81877, 81878) c6( ) c7(gh, T, aiH, ci, Hr, x, oG, il, anV, K, qM, pk, yo, aW, ou, eM, iT, vA, cj, k, m, gn, Uc, lT, oY, aj, b, kH, h, hn, avP, aq, ajo, cox, zZ, op, bo, hL, hR, fL, zl, lB, Po, B, qc, bL, ul, dA, DD, cz, v, avO, cow, er, Mg, mj, aT, jj, hK, rz, rE, ae, bA, apl, s, Y, en, cl, t, ck, ov, bt, jh, hF, mC, ad, yh, iL, ff, bBO, eZ, OG, oQ, i, y, vt, kS); #c1(PBX1) c2(NP_001191892) c3(pre-B-cell leukemia transcription factor 1 isoform 3) c4(81879) c5(81880, 81881, 81882, 81883, 81884, 81885) c6( ) c7(gh, T, aiH, ci, Hr, x, oG, il, anV, K, qM, pk, yo, aW, ou, eM, iT, vA, cj, k, m, gn, Uc, lT, oY, aj, b, kH, h, hn, avP, aq, ajo, cox, zZ, op, bo, hL, hR, fL, zl, lB, Po, B, qc, bL, ul, dA, DD, cz, v, avO, cow, er, Mg, mj, aT, jj, hK, rz, rE, ae, bA, apl, s, Y, en, cl, t, ck, ov, bt, jh, hF, mC, ad, yh, il, ff, bBO, eZ, OG, oQ, i, y, vt, kS); #c1(PBX1) c2(XP_005245285) c3 (pre-B-cell leukemia transcription factor 1 isoform X2) c4( ) c5(266394) c6( ) c7(gh, T, aiH, ci, Hr, x, oG, il, anV, K, qM, pk, yo, aW, ou, eM, iT, vA, cj, k, m, gn, Uc, lT, oY, aj, b, kH, h, hn, avP, aq, ajo, cox, zZ, op, bo, hL, hR, fL, zl, lB, Po, B, qc, bL, ul, dA, DD, cz, v, avO, cow, er, Mg, mj, aT, jj, hK, rz, rE, ae, bA, apl, s, Y, en, cl, t, ck, ov, bt, jh, hF, mC, ad, yh, il, ff, bBO, eZ, OG, oQ, i, y, vt, kS); #c1(PBX1) c2(XP_005245286) c3(pre-B-cell leukemia transcription factor 1 isoform X1) c4(266395, 266396) c5(266397, 266398, 266399, 266400, 266401, 266402, 266403) c6( ) c7(gh, T, aiH, ci, Hr, x, oG, il, anV, K, qM, pk, yo, aW, ou, eM, iT, vA, cj, k, m, gn, Uc, lT, oY, aj, b, kH, h, hn, avP, aq, ajo, cox, zZ, op, bo, hL, hR, fL, zl, lB, Po, B, qc, bL, ul, dA, DD, cz, v, avO, cow, er, Mg, mj, aT, j, hK, rz, rE, ae, bA, apL, s, Y, en, cl, t, ck, ov, bt, jh, hF, mC, ad, yh, iL, ff, bBO, eZ, OG, oQ, i, y, vt, kS); #c1(PBX1) c2(XP_005245287) c3(pre-B-cell leukemia transcription factor 1 isoform X7) c4(266404) c5(266405, 266406, 266407, 266408, 266409, 266410, 266411, 266412, 266413, 266414, 266415) c6( ) c7(gh, T, aiH, ci, Hr, x, oG, il, anV, K, qM, pk, yo, aW, ou, eM, iT, vA, cj, k, m, gn, Uc, lT, oY, aj, b, kH, h, hn, avP, aq, ajo, cox, zZ, op, bo, hL, hR, fL, zQ, lB, Po, B, qc, bL, ul, dA, DD, cz, v, avO, cow, er, Mg, mj, aT, j, hK, rz, rE, ae, bA, apL, s, Y, en, cl, t, ck, ov, bt, jh, hF, mC, ad, yh, iL, ff, bBO, eZ, OG, oQ, i, y, vt, kS); #c1(PBX1) c2(XP_006711410) c3 (pre-B-cell leukemia transcription factor 1 isoform X6) c4( ) c5(286008, 286009) c6( ) c7(gh, T, aiH, ci, Hr, x, oG, il, anV, K, qM, pk, yo, aW, ou, eM, iT, vA, cj, k, m, gn, Uc, lT, oY, aj, b, kH, h, hn, avP, aq, ajo, cox, zZ, op, bo, hL, hR, FL, zl, lB, Po, B, qc, bL, ul, dA, DD, cz, v, avO, cow, er, Mg, mj, aT, jj, hK, rz, rE, ae, bA, apL, s, Y, en, cl, t, ck, ov, bt, jh, hF, mC, ad, yh, iL, ff, bBO, eZ, OG, oQ, i, y, vt, kS); #c1(PBX1) c2(XP_011507892) c3(pre-B-cell leukemia transcription factor 1 isoform X3) c4(298156) c5(298157, 298158, 298159, 298160, 298161, 298162) c6( ) c7(gh, T, aiH, ci, Hr, x, oG, il, anV, K, qM, pk, yo, aW, ou, eM, iT, vA, cj, k, m, gn, Uc, lT, oY, aj, b, kH, h, hn, avP, aq, ajo, cox, zZ, op, bo, hL, hR, Lf, zl, lB, Po, B, qc, bL, ul, dA, DD, cz, v, avO, cow, er, Mg, mj, aT, jj, hK, rz, rE, ae, bA, apL, s, Y, en, cl, t, ck, ov, bt, jh, hF, mC, ad, yh, iL, ff, bBO, eZ, oG, oQ, i, y, vt, kS); #c1(PBX1) c2(XP_011507895) c3(pre-B-cell leukemia transcription factor 1 isoform X5) c4(298(63) c5(298164, 298165, 298166, 298167, 298168, 298169) c6( ) c7(gh, T, aiH, ci, Hr, x, oG, il, anV, K, qM, pk, yo, aW, ou, eM, iT, vA, cj, k, m, gn, Uc, lT, oY, aj, b, kH, h, hn, avP, aq, ajo, cox, zZ, op, bo, hL, hR, fL, zl, lB, Po, B, qc, bL, ul, dA, DD, cz, v, avO, cow, er, Mg, mj, aT, jj, hK, rz, rE, ae, bA, apL, s, Y, en, cl, t, ck, ov, bt, jh, hF, mC, ad, yh, iL, ff, bBO, eZ, OG, oQ, i, y, vt, kS); #c1(PBX2) c2(NP_002577) c3(pre-B-cell leukemia transcription factor 2) c4(129903, 129904, 129905, 129906, 129907) c5(129908, 129909, 129910, 129911, 129912, 129913, 129914, 129915, 129916, 129917, 129918, 129919, 129920, 129921, 129922, 129923, 129924, 129925, 129926, 129927, 129928, 129929, 129930, 129931, 129932, 129933, 129934, 129935, 129936, 129937, 129938, 129939, 129940, 129941, 129942, 129943, 129944, 129945, 129946, 129947, 129948, 129949, 129950, 129951, 129952, 129953, 129954, 129955, 129956, 129957, 129958, 129959, 129960, 129961, 129962, 129963, 129964, 129965, 129966, 129967, 129968, 129969, 129970, 129971, 129972, 129973, 129974, 129975, 129976, 129977, 129978, 129979, 129980, 129981, 129982, 129983, 129984, 129985, 129986, 129987, 129988, 129989, 129990, 129991, 129992, 129993, 129994, 129995, 129996, 129997, 129998, 129999, 130000, 130001, 130002, 130003, 130004) c6( ) c7(bL, bM, Mg, eM, Y, cr, eZ, Hn, iV, bt); #c1(PBX3) c2(NP_001128250) c3(pre-B-cell leukemia transcription factor 3 isoform 2) c4(49786, 49787) c5(49788, 49789, 49790, 49791, 49792, 49793, 49794, 49795, 49796, 49797, 49798) c6( ) c7(ck, Na, aw, Nc, gm, hp, bt, ae, ad, Hp); #c1(PBX3) c2(NP_006186) c3(pre-B-cell leukemia transcription factor 3 isoform 1) c4(162704) c5(162705, 162706, 162707, 162708, 162709, 162710, 162711) c6( ) c7(ck, Na, aw, Nc, gm, hp, bt, ae, ad, Hp); #c1(PBX3) c2(XP_006717193) c3(pre-B-cell leukemia transcription factor 3 isoform X1) c4(290792) c5(290793, 290794, 290795, 290796, 290797, 290798) c6( ) c7(ck, Na, aw, Nc, gm, hp, bt, ae, ad, Hp); #c1(PBX3) c2(XP_006717195) c3(pre-B-cell leukemia transcription factor 3 isoform X6) c4(290799) c5(290800, 290801, 290802, 290803, 290804, 290805) c6( ) c7(ck, Na, aw, Nc, gm, hp, bt, ae, ad, Hp); #c1(PBX3) c2(XP_0115/7060) c3(pre-B-cell leukemia transcription factor 3 isoform X8) c4(310549) c5(310550, 310551, 310552, 310553, 310554, 310555) c6( ) c7(ck, Na, aw, Nc, gm, hp, bt, ae, ad, Hp); #c1(PBX4) c2(NP_079521) c3(pre-B-cell leukemia transcription factor 4) c4(218569) c5(218570, 218571, 218572, 218573, 218574, 218575) c6( ) c7(x, Az, k); #c1(PC) c2(XP_011543388) c3(pyruvate carboxylase, mitochondrial isoform X1) c4(342272, 342273) c5(342274, 342275, 342276, 342277, 342278, 342279, 342280, 342281, 342282, 342283, 342284, 342285, 342286, 342287, 342288, 342289, 342290, 342291, 342292, 342293) c6( ) c7(bM, T, cg, cR, nA, pM, rc, dP, aT, K, bq, aOh, pk, is, iZ, aW, fQ, eS, bz, awp, SE, hr, cx, uJ, avB); #c1(PCBD1) c2(NP_000272) c3(pterin-4-alpha-carbinolamine dehydratase isoform 1) c4(6053) c5(6054, 6055, 6056, 6057, 6058, 6059) c6( ) c7(bL, ul, jx, fd, acD, aj, v, ame, J, ahD, coy, aU, bgF, MG, Pf, Vq, bN, DF, mf, dy, zG, LS); #c1(PCBD2) c2(NP_115527) c3(pterin-4-alpha-carbinolamine dehydratase 2) c4(223076) c5(223077, 223078, 223079, 223080, 223081, 223082, 223083, 223084, 223085, 223086) c6( ) c7(bL, bE, ld, bB, azm, BF, X); #c1(PCBP1) c2(NP_006187) c3(poly(rC)-binding protein 1) c4(162712) c5(162713, 162714, 162715, 162716, 162717, 162718) c6( ) c7(ng, T, cc, e, aT, h, aq, B); #c1(PCBP2) c2(NP_001092090) c3(poly(rC)-binding protein 2 isoform c) c4(39303) c5(39304, 39305, 39306, 39307, 39308, 39309) c6( ) c7(T, ff, jx, apn, en, ad); #c1(PCBP2) c2(NP_001122383) c3(poly(rC)-binding protein 2 isoform d) c4(46552) c5(46553, 46554, 46555, 46556, 46557, 46558) c6( ) c7(T, ff, jx, apn, en, ad); #c1(PCBP2) c2(NP_001122384) c3(poly(rC)-binding protein 2 isoform e) c4(46559) c5(46560, 46561, 46562, 46563, 46564, 46565, 46566, 46567, 46568, 46569, 46570, 46571) c6( ) c7(T, ff, jx, apn, en, ad); #c1(PCBP2) c2(NP_001122385) c3(poly(rC)-binding protein 2 isoform f) c4(46572) c5(46573, 46574, 46575, 46576, 46577, 46578) c6( ) c7(T, ff, jx, apn, en, ad); #c1(PCBP2) c2(NP_001122386) c3(poly(rC)-binding protein 2 isoform g) c4(46579) c5(46580, 46581, 46582, 46583, 46584, 46585) c6( ) c7(T, ff, jx, apn, en, ad); #c1(PCBP2) c2(NP_005007) c3(poly(rC)-binding protein 2 isoform a) c4(152028) c5(152029, 152030, 152031, 152032, 152033, 152034) c6( ) c7(T, ff, jx, apn, en, ad); #c1(PCBP2) c2(NP_114366) c3(poly(rC)-binding protein 2 isoform b) c4(222496, 222497) c5(222498, 222499, 222500, 222501, 222502, 222503) c6( ) c7(T, ff, jx, apn, en, ad); #c1(PCBP3) c2(NP_001123613) c3(poly(rC)-binding protein 3 isoform 2) c4(47815, 47816) c5(47817, 47818, 47819, 47820, 47821, 47822, 47823, 47824, 47825, 47826, 47827, 47828, 47829) c6( ) c7(bk); #c1(PCBP3) c2(NP_065389) c3(poly(rC)-binding protein 3 isoform 1) c4(204651, 204652, 204653, 204654) c5(204655, 204656, 204657, 204658, 204659, 204660, 204661, 204662, 204663, 204664, 204665, 204666, 204667, 204668, 204669, 204670, 204671, 204672, 204673, 204674, 204675, 204676, 204677, 204678, 204679, 204680, 204681, 204682) c6( ) c7(bK); #c1(PCBP4) c2(XP_005265387) c3(poly(rC)-binding protein 4 isoform X3) c4(279484, 279485, 279486, 279487, 279488, 279489, 279490) c5(279491, 279492, 279493, 279494) c6( ) c7(T, jh, it, bB, oY, fd, Kh, ci, C, nA, xg, hF, AO, aCi, q, ad, eA, av, aT, ask, ae, pi, avP, je, aW, t, bz, aU, ck, Qi, nM, iM, eM, kB, bE, CZ, m, bt, kS, L); #c1(PCBP4) c2(XP_005265388) c3(poly(rC)-binding protein 4 isoform X1) c4( ) c5(279495, 279496, 279497, 279498, 279499, 279500, 279501, 279502, 279503, 279504, 279505, 279506, 279507, 279508, 279509, 279510, 279511, 279512, 279513, 279514, 279515, 279516) c6( ) c7(T, jh, it, bB, oY, fd, Kh, ci, C, nA, xg, hF, AO, aCi, q, ad, eA, av, aT, ask, ae, pi, avP, je, aW, t, bz, all, ck, Qi, nM, iM, eM, kB, bE, CZ, m, bt, kS, L); #c1(PCBP4) c2(XP_005265389) c3(poly(rC)-binding protein 4 isoform X4) c4(279517, 279518, 279519, 279520, 279521) c5(279522, 279523, 279524) c6( ) c7(T, jh, it, bB, oY, fd, Kh, ci, C, nA, xg, hF, AO, aCi, q, ad, eA, av, aT, ask, ae, pi, avP, je, aW, t, bz, aU, ck, Qi, nM, iM, eM, kB, bE, CZ, m, bt, KS, L); #c1(PCBP4) c2(XP_006713333) c3 (poly(rC)-binding protein 4 isoform X2) c4( ) c5(287086, 287087, 287088, 287089, 287090, 287091, 287092, 287093, 287094, 287095, 287096, 287097, 287098) c6( ) c7(T, jh, it, bB, oY, fd, Kh, ci, C, nA, xg, hF, AO, aCi, q, ad, eA, av, aT, ask, ae, pi, avP, je, aW, t, bz, all, ck, Qi, nM, iM, eM, kB, bE, CZ, m, bt, kS, L); #c1(PCBP4) c2(XP_006713334) c3(poly(rC)-binding protein 4 isoform X5) c4( ) c5(287099, 287100, 287101, 287102, 287103, 287104, 287105, 287106, 287107, 287108, 287109, 287110, 287111, 287112, 287113, 287114, 287115, 287116, 287117, 287118, 287119, 287120, 287121, 287122, 287123, 287124, 287125, 287126, 287127, 287128, 287129, 287130, 287131, 287132, 287133, 287134, 287135, 287136, 287137, 287138, 287139, 287140) c6( ) c7(T, jh, it, bB, oY, fd, Kh, ci, C, nA, xg, hF, AO, aCi, q, ad, eA, av, aT, ask, ae, pi, avP, je, aW, t, bz, all, ck, Qi, nM, iM, eM, KB, bE, CZ, m, bt, kS, L); #c1(PCCA) c2(NP_000273) c3(propionyl-CoA carboxylase alpha chain, mitochondrial isoform a precursor) c4(6060) c5(6061, 6062, 6063, 6064, 6065, 6066, 6067, 6068, 6069, 6070) c6( ) c7(coz, ac, r, dq, j, g, XL); #c1(PCCA) c2(NP_001121164) c3(propionyl-CoA carboxylase alpha chain, mitochondrial isoform b) c4(45268, 45269, 45270, 45271, 45272) c5(45273, 45274, 45275, 45276, 45277, 45278, 45279, 45280, 45281, 45282, 45283, 45284, 45285, 45286, 45287, 45288, 45289, 45290, 45291, 45292, 45293, 45294, 45295, 45296) c6( ) c7(coz, ac, r, dq, j, g, XL); #c1(PCCA) c2(NP_001171475) c3(propionyl-CoA carboxylase alpha chain, mitochondrial isoform c precursor) c4(70246, 70247) c5(70248, 70249, 70250, 70251, 70252, 70253, 70254, 70255, 70256, 70257, 70258, 70259, 70260, 70261, 70262, 70263) c6( ) c7(coz, ac, r, dq, j, g, XL); #c1(PCCA) c2(XP_005254116) c3 (propionyl-CoA carboxylase alpha chain, mitochondrial isoform X2) c4(272983) c5(272984, 272985, 272986, 272987, 272988, 272989, 272990, 272991, 272992, 272993, 272994, 272995, 272996, 272997, 272998, 272999, 273000, 273001, 273002, 273003) c6(273004) c7(coz, ac, r, dq, j, g, XL); #c1(PCCA) c2(XP_011519395) c3(propionyl-CoA carboxylase alpha chain, mitochondrial isoform X1) c4( ) c5(314049) c6( ) c7(coz, ac, r, dq, j, g, XL); #c1(PCCB) c2(NP_000523) c3(propionyl-CoA carboxylase beta chain, mitochondrial isoform 1 precursor) c4(11796, 11797) c5(11798, 11799, 11800, 11801, 11802, 11803, 11804, 11805, 11806, 11807, (1808, 11809) c6( ) c7(coz, dq, g, XL, r); #c1(PCCB) c2(NP_001171485) c3(propionyl-CoA carboxylase beta chain, mitochondrial isoform 2 precursor) c4(70286) c5(70287, 70288, 70289, 70290, 70291, 70292) c6( ) c7(coz, dq, g, XL, r); #c1(PCDH1) c2(NP_001265542) c3(protocadherin-1 isoform 3) c4( ) c5(98786, 98787, 98788, 98789, 98790, 98791) c6( ) c7(bM, cT, z, sp, pM, eE); #c1(PCDH1) c2(NP_002578) c3(protocadherin-1 isoform 1 precursor) c4(130005, 130006, 130007) c5(130008, 130009, 130010, 130011, 130012, 130013, 130014, 130015, 130016, 130017, 130018, 130019) c6( ) c7(bM, cT, z, sp, pM, eE); #c1(PCDH1) c2(NP_115796) c3(protocadherin-1 isoform 2 precursor) c4(223929) c5(223930, 223931, 223932, 223933, 223934, 223935) c6( ) c7(bM, cT, z, sp, pM, eE); #c1(PCDH10) c2(NP_116586) c3(protocadherin-10 isoform 1 precursor) c4(226301, 226302) c5(226303, 226304, 226305, 226306, 226307, 226308, 226309, 226310, 226311, 226312, 226313, 226314) c6( ) c7(T, fM, dA, if, ha, jh, aA, fd, n, j, ci, nA, qN, yU, FR, q, hX, x, Bl, hb, yZ, lm, rz, pl, iU, r, dg, aF, cl, bN, Ye, ck, iT, Kr, bMt, k, UF, B); #c1(PCDH11X) c2(NP_001161832) c3(protocadherin-11 X-linked isoform e precursor) c4(66121) c5( ) c6( ) c7(Kc, iH, vJ, il, gl, aQM, af); #c1(PCDH11X) c2(NP_001161833) c3(protocadherin-11 X-linked isoform f precursor) c4(66122) c5(66123, 66124, 66125, 66126, 66127, 66128) c6( ) c7(Kc, iH, vJ, il, gl, aQM, af); #c1(PCDHllX) c2(NP_001161834) c3(protocadherin-ll X-linked isoform g precursor) c4(66129, 66130) c5(66131, 66132, 66133, 66134, 66135, 66136, 66137, 66138, 66139, 66140, 66141, 66142) c6( ) c7(Kc, iH, vJ, il, gl, aQM, af); #c1(PCDH11X) c2(NP_116751) c3(protocadherin-11 X-linked isoform d precursor) c4(226363) c5(226364, 226365, 226366, 226367, 226368) c6( ) c7(Kc, iH, vJ, il, gl, aQM, af); #c1(PCDH11X) c2(XP_011529213) c3(protocadherin-11 X-linked isoform X2) c4(324630) c5(324631, 324632) c6( ) c7(Kc, iH, vJ, il, gl, aQM, af); #c1(PCDH11X) c2(XP_011529214) c3(protocadherin-ll X-linked isoform X3) c4( ) c5(324633, 324634, 324635, 324636, 324637, 324638) c6( ) c7(Kc, iH, vJ, il, gl, aQM, af); #c1(PCDH11X) c2(XP_011529215) c3(protocadherin-11 X-linked isoform X4) c4(324639) c5(324640, 324641, 324642, 324643, 324644, 324645) c6( ) c7(Kc, iH, vJ, il, gl, aQM, af); #c1(PCDH11X) c2(XP_011529216) c3(protocadherin-11 X-linked isoform X1) c4(324646, 324647) c5(324648, 324649, 324650, 324651, 324652, 324653, 324654, 324655, 324656, 324657, 324658) c6( ) c7(Kc, iH, vJ, il, gl, aQM, af); #c1(PCDH11X) c2(XP_011529218) c3(protocadherin-11 X-linked isoform X5) c4(324659) c5(324660, 324661, 324662, 324663, 324664, 324665, 324666, 324667, 324668, 324669, 324670, 324671) c6( ) c7(Kc, iH, vJ, il, gl, aQM, af); #c1(PCDH11Y) c2(NP_116753) c3(protocadherin-11 Y-linked isoform a) c4(226369) c5(226370, 226371, 226372, 226373, 226374, 226375) c6( ) c7(gl, he, iH, ad, bN); #c1(PCDH11Y) c2(NP_116754) c3(protocadherin-11 Y-linked isoform b precursor) c4(226376) c5(226377, 226378, 226379, 226380, 226381, 226382) c6( ) c7(gl, he, iH, ad, bN); #c1(PCDH11Y) c2(NP_116755) c3(protocadherin-11 Y-linked isoform c precursor) c4(226383, 226384) c5(226385, 226386, 226387, 226388, 226389, 226390, 226391, 226392, 226393, 226394, 226395, 226396) c6( ) c7(gl, he, iH, ad, bN); #c1(PCDH15) c2(NP_001136235) c3 (protocadherin-15 isoform CDl-1 precursor) c4(54542) c5(54543, 54544, 54545, 54546, 54547, 54548) c6( ) c7(aQU, bM, alx, alz, coC, lR, af, Fi, nx, aw, aly, oc, cfl, coA, coB, y, lB, Bh, cd, nC); #c1(PCDH15) c2(NP_00136236) c3 (protocadherin-15 isoform CDl-2 precursor) c4(54549, 54550, 54551) c5(54552, 54553, 54554, 54555, 54556, 54557, 54558, 54559, 54560, 54561, 54562, 54563) c6( ) c7(aQU, bM, alx, alz, coC, lR, af, Fi, nx, aw, aly, oc, cfL, coA, coB, y, lB, Bh, cd, nC); #c1(PCDH15) c2(NP_001136237) c3 (protocadherin-15 isoform CDl-6 precursor) c4(54564, 54565, 54566) c5(54567, 54568, 54569, 54570, 54571, 54572, 54573) c6( ) c7(aQU, bM, alx, alz, coC, lR, af, Fi, nx, aw, aly, oc, cfL, coA, coB, y, lB, Bh, cd, nC); #c1(PCDH15) c2(NP_001136238) c3(protocadherin-15 isoform CDl-7 precursor) c4(54574, 54575) c5(54576, 54577, 54578, 54579, 54580, 54581) c6( ) c7(aQU, bM, alx, alz, coC, lR, af, Fi, nx, aw, aly, oc, cfL, coA, coB, y, lB, Bh, cd, nC); #c1(PCDH15) c2(NP_001136239) c3(protocadherin-15 isoform CDl-8 precursor) c4(54582) c5(54583, 54584, 54585, 54586, 54587, 54588) c6( ) c7(aQU, bM, alx, alz, coC, lR, af, Fi, nx, aw, aly, oc, cfL, coA, coB, y, lB, Bh, cd, nC); #c1(PCDH15) c2(NP_001136240) c3(protocadherin-15 isoform CD1-9 precursor) c4(54589) c5(54590, 54591, 54592, 54593, 54594, 54595) c6( ) c7(aQU, bM, alx, alz, coC, lR, af, Fi, nx, aw, aly, oc, cfl, coA, coB, y, lB, Bh, cd, nC); #c1(PCDH1S) c2(NP_001136241) c3(protocadherin-15 isoform CD2-1 precursor) c4(54596) c5(54597, 54598, 54599, 54600, 54601, 54602) c6( ) c7(aQU, bM, alx, alz, coC, lR, af, Fi, nx, aw, aly, oc, cfl, coA, coB, y, lB, Bh, cd, nC); #c1(PCDH15) c2(NP_001136242) c3(protocadherin-15 isoform CD2-2 precursor) c4(54603, 54604) c5(54605, 54606, 54607, 54608, 54609, 54610, 54611, 54612, 54613, 54614, 54615, 54616, 54617, 54618, 54619, 54620, 54621, 54622, 54623, 54624, 54625, 54626, 54627, 54628, 54629, 54630, 54631, 54632, 54633, 54634, 54635) c6( ) c7(aQU, bM, alx, alz, coC, lR, af, Fi, nx, aw, aly, oc, cfl, coA, coB, y, lB, Bh, cd, nC); #c1(PCDH15) c2(NP_001136243) c3(protocadherin-15 isoform CD3-1 precursor) c4(54636) c5(54637, 54638, 54639, 54640, 54641, 54642) c6( ) c7(aQU, bM, alx, alz, coC, lR, af, Fi, nx, aw, aly, oc, cfl, coA, coB, y, lB, Bh, cd, nC); #c1(PCDH15) c2(NP_001136244) c3 (protocadherin-15 isoform CD3-2 precursor) c4(54643, 54644, 54645) c5(54646, 54647, 54648, 54649, 54650, 54651, 54652, 54653, 54654, 54655, 54656, 54657, 54658, 54659, 54660, 54661, 54662, 54663, 54664, 54665, 54666, 54667, 54668, 54669, 54670, 54671, 54672, 54673, 54674) c6( ) c7(aQU, bM, alx, alz, coC, lR, af, Fi, nx, aw, aly, oc, cfl, coA, coB, y, lB, Bh, cd, nC); #c1(PCDH15) c2(NP_001136245) c3(protocadherin-15 isoform CDl-10 precursor) c4(54675) c5(54676, 54677, 54678, 54679, 54680, 54681) c6( ) c7(aQU, bM, alx, alz, coC, lR, af, Fi, nx, aw, aly, oc, cfL, coA, coB, y, lB, Bh, cd, nC); #c1(PCDH15) c2(NP_149045) c3(protocadherin-15 isoform CDl-4 precursor) c4(226903, 226904) c5(226905, 226906, 226907, 226908, 226909, 226910) c6( ) c7(aQU, bM, alx, alz, coC, lR, af, Fi, nx, aw, aly, oc, cfL, coA, coB, y, lB, Bh, cd, nC); #c1(PCDH17) c2(XP_005266414) c3(protocadherin-17 isoform X1) c4(279994) c5(279995, 279996, 279997, 279998, 279999, 280000) c6( ) c7(Ej, T, dL, eL, it); #c1(PCDH18) c2(NP_001287757) c3 (protocadherin-18 isoform 2 precursor) c4(112809) c5(112810, 112811, 112812, 112813, 112814, 112815) c6( ) c7(cm, eL, bA, an); #c1(PCDH18) c2(NP_061908) c3(protocadherin-18 isoform 1 precursor) c4(201154) c5(201155, 201156, 201157, 201158, 201159, 201160) c6( ) c7(cm, eZ, bA, an); #c1(PCDH19) c2(NP_001098713) c3(protocadherin-19 isoform a precursor) c4(41517) c5(41518, 41519, 41520, 41521, 41522, 41523) c6( ) c7(yr, ac, rz, hb, Kr, ha, bMt, nf, n, yZ, dg, lm, nA, coD, yU, FR, bN); #c1(PCDH19) c2(NP_001171809) c3(protocadherin-19 isoform c precursor) c4(7/798) c5(71799, 71800, 71801, 71802, 71803, 71804) c6( ) c7(yr, ac, rz, hb, Kr, ha, bMt, nf, n, yZ, dg, lm, nA, coD, yU, FR, bN); #c1(PCDH19) c2(NP_065817) c3 (protocadherin-19 isoform b precursor) c4(205678) c5(205679, 205680, 205681, 205682, 205683, 205684) c6( ) c7(yr, ac, rz, hb, Kr, ha, bMt, nf, n, yZ, dg, lm, nA, coD, yU, FR, bN); #c1(PCDH20) c2(NP_073754) c3(protocadherin-20 precursor) c4(213473, 213474) c5(213475, 213476, 213477, 213478, 213479, 213480, 213481, 213482, 213483, 213484, 213485, 213486, 213487, 213488, 213489, 213490) c6( ) c7(eM, aj, EQ); #c1(PCDH7) c2(NP_001166994) c3(protocadherin-7 isoform d precursor) c4(68776) c5(68777, 68778, 68779, 68780, 68781, 68782) c6( ) c7(Qi, ac, wL, ha, aj, dL, j, cm, fq); #c1(PCDH7) c2(NP_002580) c3(protocadherin-7 isoform a precursor) c4(130027) c5(130028, 130029, 130030, 130031, 130032, 130033) c6( ) c7(Qi, ac, wL, ha, aj, dL, j, cm, fq); #c1(PCDH7) c2(NP_115833) c3(protocadherin-7 isoform c precursor) c4(224213) c5(224214, 224215, 224216, 224217, 224218, 224219) c6( ) c7(Qi, ac, wO, ha, aj, dL, j, cm, fq); #c1(PCDH8) c2(NP_002581) c3(protocadherin-8 isoform 1 precursor) c4(130034, 130035, 130036) c5(130037, 130038, 130039, 130040, 130041, 130042, 130043, 130044, 130045, 130046, 130047, 130048, 130049, 130050, 130051, 130052, 130053, 130054) c6( ) c7(T, dD, aW, ea, aT, j, eq, fr, QK, ad); #c1(PCDH8) c2(NP_116567) c3(protocadherin-8 isoform 2 precursor) c4(226210) c5(226211, 226212, 226213, 226214, 226215, 226216) c6( ) c7(T, dD, aW, ea, aT, j, eq, fr, QK, ad); #c1(PCDH9) c2(NP_065136) c3(protocadherin-9 isoform 2 precursor) c4(204135) c5(204136, 204137, 204138, 204139, 204140, 204141, 204142, 204143, 204144, 204145) c6( ) c7(T, d, VX, dY, v, rz, eA, bN); #c1(PCDH9) c2(NP_982354) c3(protocadherin-9 isoform 1 precursor) c4(263369) c5(263370, 263371, 263372, 263373, 263374, 263375, 263376, 263377, 263378, 263379) c6( ) c7(T, d, VX, dY, v, rz, eA, bN); #c1(PCDHA1) c2(NP_061723) c3(protocadherin alpha-1 isoform 1 precursor) c4(200391) c5(200392, 200393, 200394, 200395, 200396, 200397) c6( ) c7(T, bK); #c1(PCDHA1) c2(NP_113598) c3(protocadherin alpha-isoform 2 precursor) c4(221619) c5(221620, 221621, 221622, 221623, 221624, 221625) c6( ) c7(T, bK); #c1(PCDHA1) c2(NP_113599) c3(protocadherin alpha-1 isoform 3 precursor) c4(221626) c5(221627, 221628, 221629, 221630, 221631, 221632) c6( ) c7(T, bK); #c1(PCDHA4) c2(NP_061730) c3(protocadherin alpha-4 isoform 1 precursor) c4(200398, 200399) c5(200400, 200401, 200402, 200403, 200404, 200405, 200406, 200407, 200408, 200409, 200410, 200411) c6( ) c7(bL, bO, coE, aj, bS, gf, ct, v, V, W, Vs, J, aQE, iX, Sb, bk, K, he, Gv, gC, dq, iV, Z, FX, jb, FT, pH, aap, ok, ac, cA, vK, Ei, ei, Xu, jd, ra, abW); #c1(PCDHA4) c2(NP_113688) c3(protocadherin alpha-4 isoform 2 precursor) c4(222063) c5(222064, 222065, 222066, 222067, 222068, 222069) c6( ) c7(bL, bO, coE, aj, bS, gf, ct, v, V, VV, Vs, J, aQE, iX, Sb, bK, K, he, Gv, gC, dq, iV, Z, FX, jb, FT, pH, aap, ok, ac, cA, vK, Ei, ei, Xu, jd, ra, abW); #c1 (PCDHA6) c2(NP_061732) c3(protocadherin alpha-6 isoform 1 precursor) c4(200412) c5(200413, 200414, 200415, 200416, 200417, 200418) c6( ) c7(yE, jT, af, bL, bA); #c1(PCDHA6) c2(NP_114037) c3(protocadherin alpha-6 isoform 3 precursor) c4(222102) c5(222103, 222104, 222105, 222106, 222107, 222108) c6( ) c7(yE, jT, af, bL, bA); #c1(PCDHB1) c2(NP_037472) c3(protocadherin beta-l precursor) c4(175685) c5(175686, 175687, 175688, 175689, 175690, 175691) c6( ) c7(cm, Qi); #c1(PCDHB2) c2(NP_061759) c3(protocadherin beta-2 precursor) c4(200440) c5(200441, 200442, 200443, 200444, 200445, 200446) c6( ) c7(fq); #c1(PCDHB3) c2(NP_061760) c3(protocadherin beta-3 precursor) c4(200447) c5(200448, 200449, 200450, 200451, 200452, 200453) c6( ) c7(fq); #c1(PCDHB8) c2(NP_061993) c3(protocadherin beta-8 precursor) c4(201788) c5(201789, 201790, 201791, 201792, 201793, 201794) c6( ) c7(Y); #c1(PCDHGA11) c2(NP_061737) c3(protocadherin gamma-All isoform 1 precursor) c4(200419) c5(200420, 200421, 200422, 200423, 200424, 200425) c6( ) c7(eA, as); #c1(PCDHGA11) c2(NP_114481) c3(protocadherin gamma-All isoform 3 precursor) c4(222823) c5(222824, 222825, 222826, 222827, 222828, 222829) c6( ) c7(eA, as); #c1(PCDHGA3) c2(NP_061739) c3(protocadherin gamma-A3 isoform 1 precursor) c4(200426) c5(200427, 200428, 200429, 200430, 200431, 200432) c6( ) c7(MU); #c1(PCDHGB4) c2(NP_003727) c3(protocadherin gamma-B4 isoform 1 precursor) c4(140472) c5(140473, 140474, 140475, 140476, 140477, 140478) c6( ) c7(g, s); #c1(PCDHGB6) c2(NP_061749) c3(protocadherin gamma-86 isoform 1 precursor) c4(200433) c5(200434, 200435, 200436, 200437, 200438, 200439) c6( ) c7(aT); #c1(PCDHGC3) c2(NP_002579) c3(protocadherin gamma-C3 isoform 1 precursor) c4(130020) c5(130021, 130022, 130023, 130024, 130025, 130026) c6( ) c7(q, ci, s, r); #c1(PCEDlB) c2(NP_001268358) c3(PC-esterase domain-containing protein 1B) c4(99749, 99750, 99751, 99752, 99753) c5(99754, 99755, 99756, 99757, 99758, 99759, 99760, 99761, 99762, 99763, 99764, 99765, 99766, 99767, 99768, 99769, 99770, 99771, 99772, 99773, 99774, 99775, 99776, 99777, 99778, 99779, 99780, 99781, 99782, 99783, 99784, 99785, 99786, 99787) c6( ) c7(oe, af); #c1(PCF11) c2(NP_056969) c3(pre-mRNA cleavage complex 2 protein Pcf11) c4(188248, 188249) c5(188250, 188251, 188252, 188253, 188254, 188255, 188256, 188257, 188258, 188259, 188260, 188261) c6( ) c7(h); #c1(PCGF1) c2(NP_116062) c3(polycomb group RlNG finger protein 1) c4(225311) c5(225312, 225313, 225314, 225315, 225316, 225317) c6( ) c7(y); #c1(PCGF2) c2(XP_005257698) c3(polycomb group RlNG finger protein 2 isoform X1) c4(275351) c5(275352, 275353, 275354, 275355, 275356, 275357, 275358) c6( ) c7(jf, T, aW, aT, aA, n, af, ci, ae, ad); #c1(PCGF3) c2(XP_006713915) c3(polycomb group RlNG finger protein 3 isoform X3) c4(287421) c5(287422, 287423, 287424, 287425, 287426, 287427) c6( ) c7(aHP, aHQ, fq); #c1(PCGF3) c2(XP_011511684) c3(polycomb group RlNG finger protein 3 isoform X4) c4(302397) c5( ) c6( ) c7(aHP, aHQ, fq); #c1(PCGF3) c2(XP_011511686) c3(polycomb group RlNG finger protein 3 isoform X6) c4(302398, 302399) c5( ) c6( ) c7(aHP, aHQ, fq); #c1(PCGF3) c2(XP_011511687) c3(polycomb group RlNG finger protein 3 isoform X7) c4( ) c5(302400, 302401, 302402, 302403, 302404, 302405, 302406, 302407, 302408, 302409, 302410, 302411, 302412, 302413, 302414, 302415, 302416, 302417) c6( ) c7(aHP, aHQ, fq); #c1(PCGF6) c2(NP_001011663) c3(polycomb group RlNG finger protein 6 isoform a) c4(24668) c5(24669, 24670, 24671, 24672, 24673, 24674) c6( ) c7(fM); #c1(PCGF6) c2(NP_115530) c3(polycomb group RlNG finger protein 6 isoform b) c4(223101) c5(223102, 223103, 223104, 223105, 223106, 223107) c6( ) c7(fM); #c1(PClD2) c2(NP_001120675) c3(PCI domain-containing protein 2 isoform 1) c4(44475, 44476) c5(44477, 44478, 44479, 44480, 44481, 44482, 44483, 44484, 44485, 44486, 44487, 44488, 44489, 44490, 44491, 44492, 44493, 44494, 44495, 44496) c6( ) c7(dX, bpm, eA, aUF); #c1(PClD2) c2(NP_001245141) c3(PCI domain-containing protein 2 isoform 2) c4(92598) c5(92599, 92600, 92601, 92602, 92603, 92604) c6( ) c7(dX, bpm, eA, aUF); #c1(PClD2) c2(NP_001245142) c3(PCI domain-containing protein 2 isoform 3) c4(92605, 92606) c5(92607, 92608, 92609, 92610, 92611, 92612, 92613, 92614, 92615, 92616, 92617, 92618) c6( ) c7(dX, bpm, eA, aUF); #c1(PCK1) c2(NP_002582) c3(phosphoenolpyruvate carboxykinase, cytosolic [GTP]) c4(130055, 130056, 130057) c5(130058, 130059, 130060, 130061, 130062, 130063) c6( ) c7(bL, dD, eR, af, qQ, fQ, aj, v, eq, ei, bq, B, J); #c1(PCK1) c2(XP_011527141) c3(phosphoenolpyruvate carboxykinase, cytosolic [GTP] isoform X1) c4(321638) c5(321639, 321640, 321641, 321642, 321643, 321644, 321645, 321646, 321647, 321648, 321649, 321650, 321651, 321652, 321653, 321654, 321655, 321656, 321657) c6( ) c7(bL, dD, eR, af, qQ, fQ, aj, v, eq, ei, bq, B, J); #c1(PCK2) c2(NP_001018083) c3(phosphoenolpyruvate carboxykinase [GTP], mitochondrial isoform 2 precursor) c4(27162) c5(27163, 27164, 27165, 27166, 27167, 27168) c6( ) c7(bL, T, Yn, wK, fc, NT, coF, aq, aj, mf, dG, K, cP, ei, B, J); #c1(PCK2) c2(NP_004554) c3 (phosphoenolpyruvate carboxykinase [GTP], mitochondrial isoform 1 precursor) c4(147605, 147606) c5(147607, 147608, 147609, 147610, 147611, 147612, 147613, 147614) c6( ) c7(bL, T, Yn, wK, fc, NT, coF, aq, aj, mf, dG, K, cP, ei, B, J); #c1(PCLO) c2(NP_055325) c3(protein piccolo isoform 2) c4(180490, 180491, 180492, 180493, 180494) c5(180495, 180496, 180497, 180498, 180499, 180500, 180501, 180502, 180503, 180504, 180505, 180506, 180507, 180508, 180509, 180510, 180511, 180512, 180513, 180514, 180515, 180516, 180517) c6(180518, 180519) c7(bL, cF, alC, bO, bS, bK, ct, nA, Z, bmd, bN); #c1(PCLO) c2(NP_149015) c3(protein piccolo isoform 1) c4(226720, 226721) c5(226722, 226723, 226724, 226725, 226726, 226727, 226728) c6( ) c7(bL, cF, alC, bO, bS, bK, ct, nA, Z, bmd, bN); #c1(PCM1) c2(NP_006188) c3(pericentriolar material 1 protein) c4(162719) c5(162720, 162721, 162722, 162723, 162724, 162725) c6( ) c7(ou, ul, rR, nM, nB, ci, m, Tp, coG, hF); #c1(PCM1) c2(XP_006716403) c3(pericentriolar material 1 protein isoform X6) c4(290275) c5(290276, 290277, 290278, 290279, 290280, 290281, 290282, 290283, 290284, 290285, 290286) c6( ) c7(ou, ul, rR, nM, nB, ci, m, Tp, coG, hF); #c1(PCM1) c2(XP_011542835) c3 (pericentriolar material 1 protein isoform X3) c4(341450) c5( ) c6( ) c7(ou, ul, rR, nM, nB, ci, m, Tp, coG, hF); #c1(PCM1) c2(XP_011542842) c3 (pericentriolar material 1 protein isoform X14) c4(341451) c5(341452, 341453, 341454, 341455, 341456, 341457) c6( ) c7(ou, ul, rR, nM, nB, ci, m, Tp, coG, hF); #c1(PCM1) c2(XP_011542844) c3(pericentriolar material 1 protein isoform X18) c4(341458) c5( ) c6( ) c7(ou, ul, rR, nM, nB, ci, m, Tp, coG, hF); #c1(PCMT1) c2(NP_001238978) c3(protein-L-isoaspartate(D-aspartate) O-methyltransferase isoform 2) c4(88115) c5(88116, 88117, 88118, 88119, 88120, 88121) c6( ) c7(A, lp, il, aj, zZ, J); #c1(PCMT1) c2(NP_001238980) c3 (protein-L-isoaspartate(D-aspartate) O-methyltransferase isoform 4) c4(88122) c5(88123, 88124, 88125, 88126, 88127, 88128) c6( ) c7(A, lp, il, aj, zZ, J); #c1(PCMT1) c2(NP_001238982) c3(protein-L-isoaspartate(D-aspartate) O-methyltransferase isoform 1) c4(88129, 88130) c5(88131, 88132, 88133, 88134, 88135, 88136, 88137, 88138, 88139, 88140, 88141, 88142, 88143, 88144, 88145, 88146) c6( ) c7(A, lp, il, aj, zZ, J); #c1(PCMTD1) c2(NP_001273711) c3(protein-L-isoaspartate O-methyltransferase domain-containing protein 1 isoform 2) c4(105702) c5(105703, 105704, 105705, 105706, 105707, 105708) c6( ) c7(lJ, fq, aw, sa); #c1(PCMTD1) c2(NP_443169) c3(protein-L-isoaspartate O-methyltransferase domain-containing protein 1 isoform 1) c4(229705) c5(229706, 229707, 229708, 229709, 229710, 229711, 229712, 229713, 229714, 229715) c6( ) c7(lJ, fq, aw, sa); #c1(PCNA) c2(NP_872590) c3(proliferating cell nuclear antigen) c4(256457) c5(256458, 256459, 256460, 256461, 256462, 256463, 256464, 256465, 256466, 256467) c6( ) c7(uB, gh, T, os, bM, jW, fR, aC, pf, if, e, al, fd, bB, ci, aR, ip, x, bv, afr, aE, dE, pX, Qk, ajv, K, hu, pk, dF, bc, eL, cp, DA, akV, yo, bn, aW, col, NR, g, as, zE, eF, ir, cm, bw, iP, eM, nB, AM, dc, nF, vA, U, cj, PV, iS, uJ, k, m, hh, aJi, Vj, iu, aEj, lT, wu, zt, Or, ht, Bm, n, aj, oY, fr, Qh, uE, qN, asS, br, eW, Mo, b, Bl, ft, Hx, pe, jz, kH, h, eV, Gw, dL, ey, Ha, YT, dx, pi, kj, PX, Lh, ks, ahJ, aq, kp, yq, op, iO, kT, bP, dB, hk, eD, Pl, bo, KT, hL, hR, at, MW, SZ, fL, YH, it, B, AN, cn, bL, fM, dA, AS, mr, jx, eX, aA, GC, hy, v, j, aEb, AO, V, lu, q, J, er, eA, XX, nv, hb, akU, BJ, MP, qK, aT, cy, rO, zg, aJA, aPa, jj, ae, af, eu, qw, s, vT, iU, Y, ev, ayU, LO, aEk, r, Lf, aYp, lo, en, hf, bu, t, cl, apx, ik, aHG, ck, ov, Qi, alC, Dy, nM, iM, dh, aJQ, im, bt, rA, coH, Gl, hl, id, MH, iW, ml, jh, aHv, oZ, Fb, jE, OH, hF, Fc, mC, ad, afb, hX, qC, Jm, dP, hd, av, NS, hD, vw, wm, fb, Dl, d, lx, je, qt, ff, eH, cr, eZ, Ej, aF, Vl, anW, iR, bz, LB, bF, gk, bE, Hs, aDJ, bC, Gu, i, hE, KS, X, y, BA); #c1(PCNT) c2(NP_006022) c3 (pericentrin) c4(161232, 161233) c5(161234, 161235, 161236, 161237, 161238, 161239, 161240, 161241, 161242, 161243, 161244, 161245) c6( ) c7(T, bO, fd, aHv, aj, J, av, aT, bk, he, ae, af, aii, aW, WE, QK, f, avx, col, coJ, yA, cok); #c1(PCNXL2) c2(NP_055616) c3 (pecanex-like protein 2) c4(182300, 182301) c5(182302, 182303, 182304, 182305, 182306, 182307) c6( ) c7(bL, HN, q, ac, j); #c1(PCNXL2) c2(XP_005273329) c3 (pecanex-like protein 2 isoform X1) c4(284479) c5( ) c6( ) c7(bL, HN, q, ac, j); #c1(PCNXL2) c2(XP_006711879) c3(pecanex-like protein 2 isoform X3) c4( ) c5(286270, 286271, 286272, 286273, 286274, 286275) c6( ) c7(bL, HN, q, ac, j); #c1(PCNXL2) c2(XP_011542581) c3 (pecanex-like protein 2 isoform X4) c4( ) c5(34/137, 341138, 341139, 341140, 341141, 341142) c6( ) c7(bL, HN, q, ac, j); #c1(PCNXL4) c2(NP_071940) c3(pecanex-like protein 4) c4(212423) c5(212424, 212425, 212426, 212427, 212428) c6( ) c7(Qi); #c1(PCNXL4) c2(XP_005268066) c3(pecanex-like protein 4 isoform X1) c4(281461, 281462, 281463) c5(281464, 281465, 281466, 281467, 281468, 281469, 281470, 281471, 281472, 281473, 281474, 281475, 281476, 281477, 281478, 281479, 281480, 281481, 281482, 281483, 281484, 281485) c6( ) c7(Qi); #c1(PCOLCE) c2(NP_002584) c3(procollagen C-endopeptidase enhancer 1 precursor) c4(130064) c5(130065, 130066, 130067, 130068, 130069, 130070) c6( ) c7(dw); #c1(PCOLCE2) c2(NP_037495) c3(procollagen C-endopeptidase enhancer 2 precursor) c4(175838) c5(175839, 175840, 175841, 175842, 175843, 175844) c6( ) c7(xa, dH); #c1(PCP2) c2(NP_777555) c3(Purkinje cell protein 2 homolog isoform 1) c4(25/129) c5(251130, 251131, 251132, 251133, 251134, 251135) c6( ) c7(eJ, rU); #c1(PCP4) c2(NP_006189) c3(Purkinje cell protein 4) c4(162726) c5(162727, 162728, 162729, 162730, 162731, 162732) c6( ) c7(s, hW, vA, f, Fg, X, op); #c1(PCSK1) c2(NP_000430) c3(neuroendocrine convertase 1 isoform 1 preproprotein) c4(8895, 8896) c5(8897, 8898, 8899, 8900, 8901, 8902, 8903, 8904, 8905, 8906, 8907, 8908) c6( ) c7(bL, aec, aw, qQ, hE, cz, jh, aj, dG, j, v, OH, J, mj, il, nX, dL, bK, qM, jo, bq, coN, af, kl, A, coM, adF, DL, fq, ag, hr, ei, bD, gT, adG, y); #c1(PCSK1) c2(NP_001171346) c3(neuroendocrine convertase 1 isoform 2) c4(70048) c5(70049, 70050, 70051, 70052, 70053, 70054) c6( ) c7(bL, aec, aw, qQ, hE, cz, jh, aj, dG, j, v, OH, J, mj, il, nX, dL, bK, qM, jo, bq, coN, af, kl, A, coM, adF, DL, fq, ag, hr, ei, bD, gT, adG, y); #c1(PCSK1N) c2(NP_037403) c3(proSAAS preproprotein) c4(175327) c5(175328, 175329, 175330, 175331, (75332, 175333) c6( ) c7(bb, v, af, qK); #c1(PCSK2) c2(NP_001188457) c3(neuroendocrine convertase 2 isoform 3) c4( ) c5(79191, 79192, 79193, 79194, 79195, 79196) c6( ) c7(bL, fU, yo, qJ, ac, c, s, nB, dY, aw, jh, lG, v, dy, jo, bq, OH, af); #c1(PCSK2) c2(NP_00188458) c3(neuroendocrine convertase 2 isoform 2 preproprotein) c4(79197) c5(79198, 79199, 79200, 79201, 79202, 79203) c6( ) c7(bL, fU, yo, qJ, ac, c, s, nB, dY, aw, jh, lG, v, dy, jo, bq, OH, af); #c1(PCSK2) c2(NP_002585) c3(neuroendocrine convertase 2 isoform 1 preproprotein) c4(130071, 130072) c5(130073, 130074, 130075, 130076, 130077, 130078, 130079) c6( ) c7(bL, fU, yo, qJ, ac, c, s, nB, dY, aw, jh, lG, v, dy, jo, bq, OH, af); #c1(PCSK4) c2(NP_060043) c3(proprotein convertase subtilisin/kexin type 4 preproprotein) c4(193854) c5(193855, 193856, 193857, 193858, 193859, 193860) c6( ) c7(ci, eM); #c1(PCSK4) c2(XP_011526389) c3(proprotein convertase subtilisin/kexin type 4 isoform X3) c4( ) c5(320664) c6( ) c7(ci, eM); #c1(PCSK4) c2(XP_011526395) c3(proprotein convertase subtilisin/kexin type 4 isoform X9) c4(320665) c5(320666, 320667, 320668, 320669, 320670) c6( ) c7(ci, eM); #c1(PCSK4) c2(XP_011526397) c3(proprotein convertase subtilisin/kexin type 4 isoform X11) c4( ) c5(320671, 320672) c6( ) c7(ci, eM); #c1(PCSK5) c2(NP_001177411) c3(proprotein convertase subtilisin/kexin type 5 isoform PCGB preproprotein) c4(72773, 72774, 72775) c5(72776, 72777, 72778, 72779, 72780, 72781) c6( ) c7(gx, dD, bDg, coO, bKP, gY, ea, bjd, acl, agw, Ll, aSp, wR, dy, blm, eq, Lk, V, aa, bOA); #c1(PCSK5) c2(NP_006191) c3(proprotein convertase subtilisin/kexin type 5 isoform PCGA preproprotein) c4(162733, 162734) c5(162735, 162736, 162737, 162738, 162739, 162740, 162741, 162742, 162743, 162744, 162745, 162746) c6( ) c7(gx, dD, bDg, col, bKP, gY, ea, bjd, acl, agw, Ll, aSp, wR, dy, blm, eq, Lk, V, aa, bOA); #c1(PCSK5) c2(XP_005252096) c3(proprotein convertase subtilisin/kexin type 5 isoform X1) c4(271540, 271541) c5(271542, 271543, 271544, 271545, 271546, 271547, 271548, 271549, 271550, 271551, 271552, 271553) c6( ) c7(gx, dD, bDg, coO, bKP, gY, ea, bjd, acl, agw, Ll, aSp, wR, dy, blm, eq, Lk, V, aa, bOA); #c1(PCSK5) c2(XP_011517071) c3(proprotein convertase subtilisin/kexin type 5 isoform X2) c4( ) c5(310581, 310582, 310583, 310584, 310585, 310586, 310587, 310588, 310589, 310590, 310591, 310592) c6( ) c7(gx, dD, bDg, coO, bKP, gY, ea, bjd, acl, agw, Ll, aSp, wR, dy, blm, eq, Lk, V, aa, bOA); #c1 (PCSK6) c2(NP_002561) c3 (proprotein convertase subtilisin/kexin type 6 isoform PACE4-Al preproprotein) c4(129812) c5(129813, 129814, 129815, 129816, 129817, 129818) c6( ) c7(T, dD, Fn, d, aW, hk, oe, ea, aT, cm, cS, m, coP, eq, adg, t, yA, ae, eA, ad); #c1(PCSK6) c2(NP_612192) c3(proprotein convertase subtilisin/kexin type 6 isoform PACE4-All preproprotein) c4(236149) c5(236150, 236151, 236152, 236153, 236154, 236155) c6( ) c7(T, dD, Fn, d, aW, hk, oe, ea, aT, cm, cS, m, coP, eq, adg, t, yA, ae, eA, ad); #c1(PCSK6) c2(NP_612196) c3(proprotein convertase subtilisin/kexin type 6 isoform PACE4CS preproprotein) c4(236156) c5(236157, 236158, 236159, 236160, 236161, 236162) c6( ) c7(T, dD, Fn, d, aW, hk, oe, ea, aT, cm, cS, m, coP, eq, adg, t, yA, ae, eA, ad); #c1(PCSKG) c2(NP_612197) c3(proprotein convertase subtilisin/kexin type 6 isoform PACE4C preproprotein) c4(236163) c5(236164, 236165, 236166, 236167, 236168, 236169) c6( ) c7(T, dD, Fn, d, aW, hk, oe, ea, aT, cm, cS, m, coP, eq, adg, t, yA, ae, eA, ad); #c1(PCSK6) c2(NP_612198) c3(proprotein convertase subtilisin/kexin type 6 isoform f preproprotein) c4(236170) c5(236171, 236172, 236173, 236174, 236175, 236176, 236177, 236178, 236179, 236180, 236181) c6( ) c7(T, dD, Fn, d, aW, hk, oe, ea, aT, cm, cS, m, coP, eq, adg, t, yA, ae, eA, ad); #c1(PCSK7) c2(NP_004707) c3(proprotein convertase subtilisin/kexin type 7 preproprotein) c4(148974, 148975) c5(148976, 148977, 148978, 148979, 148980, 148981, 148982, 148983, 148984, 148985, 148986, 148987, 148988) c6( ) c7(gk, aW, al, aT, yu, fr, bt, dF); #c1(PCSK7) c2(XP_006719002) c3(proprotein convertase subtilisin/kexin type 7 isoform X1) c4(292150, 292151, 292152) c5( ) c6( ) c7(gk, aW, aT, aT, yu, fr, bt, dF); #c1(PCSK7) c2(XP_006719003) c3 (proprotein convertase subtilisin/kexin type 7 isoform X4) c4( ) c5(292153, 292154, 292155, 292156, 292157) c6( ) c7(gk, aW, al, aT, yu, fr, bt, dF); #c1(PCSK7) c2(XP_011541364) c3(proprotein convertase subtilisin/kexin type 7 isoform X3) c4( ) c5(339951, 339952, 339953, 339954, 339955, 339956, 339957, 339958, 339959, 339960, 339961, 339962, 339963, 339964, 339965, 339966) c6( ) c7(gk, aW, aT, al, yu, fr, bt, dF); #c1(PCTP) c2(NP_001095872) c3 (phosphatidylcholine transfer protein isoform 2) c4( ) c5(40870, 40871, 40872, 40873, 40874) c6( ) c7(hJ, jb, dL, at); #c1(PCTP) c2(NP_067036) c3(phosphatidylcholine transfer protein isoform 1) c4(208550, 208551, 208552) c5(208553, 208554, 208555, 208556, 208557, 208558, 208559, 208560, 208561, 208562, 208563, 208564, 208565) c6( ) c7(hJ, jb, dL, at); #c1(PCYT1A) c2(NP_005008) c3(choline-phosphate cytidylyltransferase A) c4(152035, 152036, 152037, 152038, 152039) c5(152040, 152041, 152042, 152043, 152044, 152045, 152046, 152047, 152048, 152049, 152050, 152051, 152052, 152053, 152054, 152055, 152056, 152057, 152058, 152059, 152060, 152061, 152062, 152063, 152064, 152065, 152066, 152067, 152068, 152069, 152070, 152071, 152072, 152073) c6( ) c7(Mb, T, coQ, qX, wu, aO, ht, l, fd, rf, j, eA, ad, Na, aT, Nc, wm, an, hu, ae, af, bf, iU, vT, aW, Y, bu, KQ, ky, kB, lp, iT, y, bt, B); #c1(PCYT1B) c2(NP_001156736) c3(choline-phosphate cytidylyltransferase B isoform 2) c4(61865) c5(61866, 61867, 61868, 61869, 61870, 61871) c6( ) c7(jN); #c1(PCYT1B) c2(NP_001156737) c3(choline-phosphate cytidylyltransferase B isoform 3) c4(61872) c5(61873, 61874, 61875, 61876, 61877, 61878) c6( ) c7(jN); #c1(PCYT1B) c2(NP_004836) c3(choline-phosphate cytidylyltransferase B isoform 1) c4(150174) c5(150175, 150176, 150177, 150178, 150179, 150180) c6( ) c7(jN); #c1(PDAP1) c2(NP_055706) c3(28 kDa heat- and acid-stable phosphoprotein) c4(182952) c5(182953, 182954, 182955, 182956, 182957, 182958) c6( ) c7(T, dA, if, iW, aj, v, qN, cd, b, aoA, zl, d, xX, r, uF, GR, ck, wq, dT, zG, i, ao); #c1(PDC) c2(NP_072098) c3(phosducin isoform b) c4( ) c5(212702, 212703, 212704, 212705, 212706, 212707, 212708, 212709) c6( ) c7(btY, cV, cz, fd, v, fr, bh, oc, bkU, cd, ahR, Hx, hd, oW, aT, dV, ji, K, bc, lR, Jl, nf, kt, t, nC, xw, ck, c, vA, m, bt, L); #c1(PDC) c2(XP_011507905) c3(phosducin isoform X1) c4(298193, 298194) c5(298195, 298196, 298197, 298198, 298199, 298200, 298201, 298202, 298203, 298204, 298205, 298206) c6( ) c7(btY, cV, cz, fd, v, fr, bh, oc, bkU, cd, ahR, Hx, hd, oW, aT, dV, ji, K, bc, lR, Jl, nf, kt, t, nC, xw, ck, c, vA, m, bt, L); #c1(PDCD1) c2(NP_005009) c3(programmed cell death protein 1 precursor) c4(152074, 152075) c5(152076, 152077, 152078, 152079, 152080, 152081, 152082, 152083, 152084, 152085, 152086, 152087) c6( ) c7(Ob, Kd, T, fR, lo, jx, coR, fN, aw, eX, jE, ci, uE, qN, V, mC, ai, ip, hX, ga, KH, aT, alO, hD, h, eV, hH, nJ, bc, hM, A, mR, CN, iU, xR, qZ, aW, Y, je, HA, hl, cr, eZ, fe, lo, hp, iV, ve, iM, mx, uo, aYx, dW, dy, fL, eo, ME, y, hv); #c1(PDCD10) c2(NP_665859) c3(programmed cell death protein 10) c4(241790, 24/791, 241792, 241793, 24/794, 24/795) c5(241796, 24/797, 241798, 241799, 241800, 241801, 241802, 241803, 241804, 241805, 241806, 241807, 241808, 241809, 241810, 241811, 241812, 241813, 241814, 241815, 241816, 241817, 241818, 241819, 241820, 241821, 241822, 241823, 241824, 241825, 241826, 241827, 241828, 241829, 241830, 241831, 241832, 241833, 241834, 241835, 241836, 241837, 241838, 241839, 241840, 241841, 241842, 241843, 241844, 241845, 241846, 241847) c6( ) c7(T, cF, hb, dB, Ot, K, coS); #c1(PDCD1LG2) c2(NP_079515) c3(programmed cell death l ligand 2 precursor) c4(218510, 218511) c5(218512, 218513, 218514, 218515, 218516, 218517, 218518, 218519, 218520, 218521, 218522, 218523, 218524, 218525, 218526, 218527) c6(218528, 218529, 218530, 218531) c7(fR, ht, al, eX, aA, bh, V, h, hD, eV, hu, hH, bc, A, mR, qZ, Y, ff, hl, eZ, r, aF, en, bw, DF, cj, eo, y, bt); #c1(PDCD2) c2(NP_001186391) c3 (programmed cell death protein 2 isoform 4) c4(77604, 77605) c5(77606, 77607, 77608, 77609, 77610, 77611) c6( ) c7(ck, T, A, fM, eM, jh, ci, g, hF, bz, iu); #c1(PDCD2) c2(NP_00186392) c3(programmed cell death protein 2 isoform 5) c4(77612) c5(77613, 77614, 77615, 77616, 77617, 77618) c6( ) c7(ck, T, A, fM, eM, jh, ci, g, hF, bz, iu); #c1(PDCD2) c2(NP_001186393) c3(programmed cell death protein 2 isoform 6) c4( ) c5(77619, 77620, 77621, 77622, 77623, 77624) c6( ) c7(ck, T, A, fM, eM, jh, ci, g, hF, bz, iu); #c1(PDCD2) c2(NP_002589) c3(programmed cell death protein 2 isoform 1) c4(130093) c5(130094, 130095, 130096, 130097, 130098, 130099) c6( ) c7(ck, T, A, fM, eM, jh, ci, g, hF, bz, iu); #c1(PDCD2) c2(NP_659005) c3(programmed cell death protein 2 isoform 2) c4(239709) c5(239710, 239711, 239712, 239713, 239714, 239715) c6( ) c7(ck, T, A, fM, eM, jh, ci, g, hF, bz, iu); #c1(PDCD2) c2(XP_011534180) c3(programmed cell death protein 2 isoform X1) c4( ) c5(330484) c6( ) c7(ck, T, A, fM, eM, jh, ci, g, hF, bz, iu); #c1(PDCD4) c2(NP_001186421) c3(programmed cell death protein 4 isoform 3) c4(77630) c5(77631, 77632, 77633, 77634, 77635, 77636) c6( ) c7(bM, T, fM, AS, aA, bB, ci, pM, atK, mC, q, ad, eA, av, Bl, jz, Do, hW, yt, nv, Db, aT, hP, K, ji, hu, jj, ae, amw, jf, xQ, d, B, s, aeR, mR, aW, ev, ff, eZ, r, aq, agF, kp, aF, en, mo, pL, t, g, bz, iR, op, es, iA, nB, dB, bE, vA, cj, m, fL, lC, bt, it, X); #c1(PDCD4) c2(NP_055271) c3(programmed cell death protein 4 isoform 1) c4(180190) c5(180191, 180192, 180193, 180194, 180195, 180196, 180197) c6( ) c7(bM, T, fM, AS, aA, bB, ci, pM, atK, mC, q, ad, eA, av, Bl, jz, Do, hW, yt, nv, Db, aT, hP, K, ji, hu, jj, ae, amw, jf, xQ, d, B, s, aeR, mR, aW, ev, ff, eZ, r, aq, agF, kp, aF, en, mo, pL, t, g, bz, iR, op, es, iA, nB, dB, bE, vA, cj, m, fL, lC, bt, it, X); #c1(PDCD4) c2(NP_663314) c3 (programmed cell death protein 4 isoform 2) c4(241192) c5(241193, 241194, 241195, 241196, 241197) c6( ) c7(bM, T, fM, AS, aA, bB, ci, pM, atK, mC, q, ad, eA, av, Bl, jz, Do, hW, yt, nv, Db, aT, hP, K, ji, hu, jj, ae, amw, jf, xQ, d, B, s, aeR, mR, aW, ev, ff, eZ, r, aq, agF, kp, aF, en, mo, pL, t, g, bz, iR, op, es, iA, nB, dB, bE, vA, cj, m, fL, lC, bt, it, X); #c1(PDCD5) c2(NP_004699) c3 (programmed cell death protein 5) c4(148906, 148907) c5(148908, 148909, 148910, 148911, 148912, 148913, 148914, 148915, 148916, 148917, 148918, 148919, 148920, 148921, 148922, 148923) c6( ) c7(ck, ai, T, kh, iu, iU, eH, r, aA, aF, q, as, eA, eF, y, bt, bz, B, hX); #c1(PDCD5) c2(XP_005259449) c3(programmed cell death protein 5 isoform X1) c4(276104) c5(276105, 276106, 276107, 276108, 276109, 276110) c6( ) c7(ck, ai, T, kh, iu, iU, eH, r, aA, aF, q, as, eA, eF, y, bt, bz, B, hX); #c1(PDCD6) c2(NP_001254485) c3(programmed cell death protein 6 isoform 2) c4(94456) c5(94457, 94458, 94459, 94460, 94461, 94462) c6( ) c7(av, T, xA, bE, eL, Y, aA, bB, n, dL, aF, m, t, bz, iP); #c1(PDCD6) c2(NP_001254486) c3(programmed cell death protein 6 isoform 3) c4(94463) c5( ) c6( ) c7(av, T, xA, bE, eL, Y, aA, bB, n, dL, aF, m, t, bz, P); #c1(PDCD6) c2(NP_001254488) c3(programmed cell death protein 6 isoform 5) c4(94464, 94465) c5(94466, 94467, 94468, 94469, 94470, 94471, 94472, 94473, 94474, 94475, 94476, 94477, 94478, 94479, 94480, 94481, 94482, 94483) c6( ) c7(av, T, xA, bE, eL, Y, aA, bB, n, dL, aF, m, t, bz, iP); #c1(PDCD6) c2(NP_037364) c3(programmed cell death protein 6 isoform 1) c4(175030) c5(175031, 175032, 175033, 175034, 175035, 175036) c6( ) c7(av, T, xA, bE, eL, Y, aA, bB, n, dL, aF, m, t, bz, iP); #c1(PDCD6IP) c2(NP_001155901) c3(programmed cell death 6-interacting protein isoform 2) c4(61641) c5(61642, 61643, 61644, 61645, 61646, 61647) c6( ) c7(bL, T, ac, eM, ea, Y, h, ey, Ea, bz, cC); #c1(PDCDG1P) c2(NP_037506) c3(programmed cell death 6-interacting protein isoform 1) c4(175903) c5(175904, 175905, 175906, 175907, 175908, 175909) c6( ) c7(bL, T, ac, eM, ea, Y, h, ey, Ea, bz, cC); #c1(PDCD7) c2(NP_005698) c3(programmed cell death protein 7) c4(158612, 158613, 158614) c5(158615, 158616, 158617, 158618, 158619, 158620, 158621, 158622, 158623, 158624, 158625, 158626, 158627, 158628, 158629, 158630, 158631, 158632) c6( ) c7(bt); #c1(PDCD7) c2(XP_005254165) c3(programmed cell death protein 7 isoform X1) c4( ) c5(273042) c6( ) c7(bt); #c1(PDCL2) c2(NP_689614) c3(phosducin-like protein 2) c4(243267) c5(243268, 243269, 243270, 243271, 243272, 243273) c6( ) c7(aF, aA); #c1(PDCL3) c2(NP_076970) c3(phosducin-like protein 3) c4(214522) c5(214523, 214524, 214525, 214526, 214527, 214528) c6( ) c7(dS); #c1(PDE1DA) c2(NP_001124162) c3(cAMP and cAMP-inhibited cGMP 3′,5′-cyclic phosphodiesterase 10A) c4(48347, 48348) c5(48349, 48350, 48351, 48352, 48353, 48354, 48355, 48356, 48357, 48358, 48359) c6( ) c7(c, aw, lC, bjf, U, HK, gl, uJ, v, bK, j, hz, qj, Gp, gT, aen, aR, hq); #c1(PDE11A) c2(NP_001070664) c3(dual 3′,5′-cyclic-AMP and -GMP phosphodiesterase 11A isoform 1) c4( ) c5(34660, 34661, 34662, 34663, 34664, 34665) c6( ) c7(bM, T, gU, vf, bO, Ja, JW, ad, aeZ, iv, avH, s, vT, col, md, iw, bbe, Hu, bu, Z, Ld, bLF, hR, HB, Lb); #c1(PDEN1A) c2(NP_001070665) c3(dual 3′,5′-cyclic-AMP and -GMP phosphodiesterase (1A isoform 3) c4(34666, 34667) c5(34668, 34669, 34670, 34671, 34672, 34673, 34674, 34675, 34676, 34677, 34678, 34679, 34680, 34681, 34682, 34683, 34684, 34685, 34686, 34687, 34688, 34689, 34690, 34691, 34692, 34693, 34694, 34695) c6( ) c7(bM, T, gU, vf, bO, Ja, JW, ad, aeZ, iv, avH, s, vT, col, mQ, iw, bbe, Hu, bu, Z, Ld, bLF, hR, HB, Lb); #c1(PDE11A) c2(NP_001070826) c3(dual 3′,5′-cyclic-AMP and -GMP phosphodiesterase 11A isoform 2) c4(34850, 34851, 34852) c5( ) c6( ) c7(bM, T, gU, vf, bO, Ja, JW, ad, aeZ, iv, avH, s, vT, coT, mQ, iw, bbe, Hu, bu, Z, Ld, bLF, hR, HB, Lb); #c1(PDE1A) c2(NP_058649) c3(dual 3′,5′-cyclic-AMP and -GMP phosphodiesterase 11A isoform 4) c4(193013) c5(193014, 193015, 193016, 193017, 193018, 193019) c6( ) c7(bM, T, gU, vf, bO, Ja, JW, ad, aeZ, iv, avH, s, vT, coT, mQ, iw, bbe, Hu, bu, Z, Ld, bLF, HR, HB, Lb); #c1(PDE12) c2(NP_808881) c3(2′,5′-phosphodiesterase 12) c4(252995) c5(252996, 252997, 252998, 252999, 253000, 253001) c6( ) c7(d); #c1(PDE12) c2(XP_011531777) c3(2′,5′-phosphodiesterase 12 isoform X1) c4(327860) c5(327861, 327862, 327863, 327864, 327865, 327866) c6( ) c7(d); #c1(PDE1A) c2(NP_001003683) c3(calcium/calmodulin-dependent 3′,5′-cyclic nucleotide phosphodiesterase 1A isoform 2) c4(19886) c5(19887, 19888, 19889, 19890, 19891, 19892, 19893, 19894, 19895, 19896) c6( ) c7(x, dD, yP, eq, k, Z); #c1(PDE1A) c2(NP_001245241) c3(calcium/calmodulin-dependent 3′,5′-cyclic nucleotide phosphodiesterase lA isoform 3) c4(92793, 92794, 92795, 92796) c5(92797) c6( ) c7(x, dD, yP, eq, k, Z); #c1(PDE1A) c2(NP_001245242) c3(calcium/calmodulin-dependent 3′,5′-cyclic nucleotide phosphodiesterase 1A isoform 4) c4(92798) c5(92799, 92800, 92801, 92802, 92803, 92804) c6( ) c7(x, dD, yP, eq, k, Z); #c1(PDE1A) c2(NP_001245243) c3(calcium/calmodulin-dependent 3′,5′-cyclic nucleotide phosphodiesterase 1A isoform 5) c4(92805) c5(92806, 92807, 92808, 92809) c6( ) c7(x, dD, yP, eq, k, Z); #c1(PDE1A) c2(NP_005010) c3(calcium/calmodulin-dependent 3′,5′-cyclic nucleotide phosphodiesterase 1A isoform 1) c4( ) c5(152088, 152089, 152090, 152091, 152092, 152093, 152094, 152095, 152096, 152097, 152098, 152099, 152100, 152101, 152102, 152103, 152104, 152105, 152106, 152107, 152108, 152109, 152110, 152111, 152112, 152113, 152114) c6( ) c7(x, dD, yP, eq, k, Z); #c1(PDE1A) c2(XP_011509628) c3(calcium/calmodulin-dependent 3′,5′-cyclic nucleotide phosphodiesterase 1A isoform X4) c4( ) c5(300205, 300206, 300207, 300208, 300209, 300210) c6( ) c7(x, dD, yP, eq, k, Z); #c1(PDE1B) c2(NP_000915) c3(calcium/calmodulin-dependent 3′,5′-cyclic nucleotide phosphodiesterase 1B isoform 1) c4(17156) c5(17157, 17158, 17159, 17160, 17161, (7162) c6( ) c7(aen, xt, cj); #c1(PDE1B) c2(NP_001159447) c3(calcium/calmodulin-dependent 3′,5′-cyclic nucleotide phosphodiesterase (B isoform 2) c4(64091) c5(64092, 64093, 64094, 64095, 64096, 64097, 64098) c6( ) c7(aen, xt, cj); #c1(PDE1B) c2(NP_001275698) c3(calcium/calmodulin-dependent 3′,5′-cyclic nucleotide phosphodiesterase (B isoform 4) c4(107116) c5(107/17, 107118, 107119, 107120, 107121) c6( ) c7(aen, xt, cj); #c1(PDE1C) c2(NP_001177986) c3(calcium/calmodulin-dependent 3′,5′-cyclic nucleotide phosphodiesterase 1C isoform 2) c4(73262) c5(73263, 73264, 73265, 73266, 73267, 73268, 73269, 73270, 73271, 73272) c6( ) c7(lB, lx); #c1(PDE1C) c2(NP_001177987) c3(calcium/calmodulin-dependent 3′,5′-cyclic nucleotide phosphodiesterase 1C isoform 3) c4(73273) c5(73274, 73275, 73276, 73277, 73278, 73279) c6( ) c7(lB, lx); #c1(PDE1C) c2(NP_005011) c3(calcium/calmodulin-dependent 3′,5′-cyclic nucleotide phosphodiesterase 1C isoform 1) c4(152115, 152116) c5(152117, 152118, 152119, 152120, 152121, 152122, 152123, 152124, 152125, 152126, 152127, 152128, 152129, 152130, 152131, 152132) c6( ) c7(lB, lx); #c1(PDE2A) c2(NP_001137311) c3(CGMP-dependent 3′,5′-cyclic phosphodiesterase isoform PDE2A2 precursor) c4(55455, 55456) c5(55457, 55458, 55459, 55460, 55461, 55462, 55463, 55464, 55465, 55466, 55467, 55468) c6( ) c7(r, q, y, C, aT); #c1(PDE2A) c2(NP_001139681) c3(cGMP-dependent 3′,5′-cyclic phosphodiesterase isoform PDE2A4) c4(59695, 59696) c5(59697, 59698, 59699, 59700, 59701, 59702) c6( ) c7(r, q, y, C, aT); #c1(PDE2A) c2(NP_002590) c3(cGMP-dependent 3′,5′-cyclic phosphodiesterase isoform PDE2A3) c4(130100) c5(130101, 130102, 130103, 130104, 130105, 130106, 130107, 130108, 130109, 130110, 130111, 130112) c6( ) c7(r, q, y, C, aT); #c1(PDE3A) c2(NP_000912) c3(cGMP-inhibited 3′,5′-cyclic phosphodiesterase A isoform 1) c4(17135) c5(17136, 17137, 17138, 17139, 17140, 17141) c6( ) c7(gU, ND, e, aw, cz, vH, bq, lC, cd, y); #c1(PDE3B) c2(NP_000913) c3(cGMP-inhibited 3′,5′-cyclic phosphodiesterase B) c4(17142, 17143) c5(17144, 17145, 17146, 17147, 17148, 17149, 17150, 17151, 17152, 17153, 17154, 17155) c6( ) c7(bL, lD, x, kl, lC, eZ, cj, h, k, as, eE, gT, Fc, vN, JR, BY); #c1(PDE4A) c2(NP_001104777) c3(cAMP-specific 3′,5′-cyclic phosphodiesterase 4A isoform 1) c4(42282) c5(42283, 42284, 42285, 42286, 42287, 42288) c6( ) c7(bM, T, Dw, e, bO, jh, adk, bS, j, ct, q, ad, HF, adD, ae, aa, je, sp, Y, asD, r, HL, Z, bN, iM, dB, z, U, cj, at, fD); #c1(PDE4A) c2(NP_001104778) c3(cAMP-specific 3′,5′-cyclic phosphodiesterase 4A isoform 2) c4(42289) c5(42290, 42291, 42292, 42293, 42294, 42295) c6( ) c7(bM, T, Dw, e, bO, jh, adk, bS, j, ct, q, ad, HF, adD, ae, aa, je, sp, Y, asD, r, HL, Z, bN, iM, dB, z, U, cj, at, fD); #c1(PDE4A) c2(NP_00104779) c3(cAMP-specific 3′,5′-cyclic phosphodiesterase 4A isoform 3) c4(42296, 42297) c5(42298, 42299, 42300, 42301, 42302, 42303) c6( ) c7(bM, T, Dw, e, bO, jh, adk, bS, j, ct, q, ad, HF, adD, ae, aa, je, sp, Y, asD, r, HL, Z, bN, iM, dB, z, U, cj, at, fD); #c1(PDE4A) c2(NP_001230050) c3(cAMP-specific 3′,5′-cyclic phosphodiesterase 4A isoform 5) c4(85852) c5(85853, 85854, 85855, 85856, 85857, 85858, 85859) c6( ) c7(bM, T, Dw, e, bO, jh, adk, bS, j, ct, q, ad, HF, adD, ae, aa, je, sp, Y, asD, r, HL, Z, bN, iM, dB, z, U, cj, at, fD); #c1(PDE4A) c2(NP_006193) c3(cAMP-specific 3′,5′-cyclic phosphodiesterase 4A isoform 4) c4(162772) c5(162773, 162774, 162775, 162776, 162777, 162778) c6( ) c7(bM, T, Dw, e, bO, jh, adk, bS, j, ct, q, ad, HF, adD, ae, aa, je, sp, Y, asD, r, HL, Z, bN, iM, dB, z, U, cj, at, fD); #c1(PDE4A) c2(XP_005259990) c3(cAMP-specific 3′,5′-cyclic phosphodiesterase 4A isoform X3) c4( ) c5(276366, 276367, 276368, 276369, 276370) c6( ) c7(bM, T, Dw, e, bO, jh, adk, bS, j, ct, q, ad, HF, adD, ae, aa, je, sp, Y, asD, r, HL, Z, bN, iM, dB, z, U, cj, at, fD); #c1(PDE4A) c2(XP_011526357) c3(cAMP-specific 3′,5′-cyclic phosphodiesterase 4A isoform X2) c4(320650) c5(320651, 320652, 320653, 320654, 320655, 320656, 320657) c6( ) c7(bM, T, Dw, e, bO, jh, adk, bS, j, ct, q, ad, HF, adD, ae, aa, je, sp, Y, asD, r, HL, Z, bN, iM, dB, z, U, cj, at, fD); #c1(PDE4D) c2(NP_001098101) c3(cAMP-specific 3′,5′-cyclic phosphodiesterase 4D isoform PDE4D4) c4(41336) c5(41337, 41338, 41339, 41340, 41341, 41342) c6( ) c7(bM, dD, eR, ea, aw, ht, cz, oR, aj, v, j, ct, nA, hz, eq, agJ, ad, dZ, bri, rS, M, EL, coV, eN, xa, eu, d, vs, pT, Kp, ek, EJ, pH, wg, ac, yl, xZ, cj, at, cS, aS, ei, coU); #c1(PDE4D) c2(NP_001159371) c3(cAMP-specific 3′,5′-cyclic phosphodiesterase 4D isoform PDE4D7) c4(63887, 63888, 63889) c5(63890, 63891, 63892, 63893, 63894, 63895, 63896, 63897, 63898, 63899, 63900, 63901, 63902, 63903, 63904, 63905, 63906, 63907, 63908, 63909, 63910, 63911, 63912) c6( ) c7(bM, dD, eR, ea, aw, ht, cz, oR, aj, v, j, ct, nA, hz, eq, agJ, ad, dZ, bri, rS, M, EL, coV, eN, xa, eu, d, vs, pT, Kp, ek, EJ, pH, wg, ac, yl, xZ, cj, at, cS, aS, ei, coU); #c1(PDE4D) c2(NP_001184147) c3(cAMP-specific 3′,5′-cyclic phosphodiesterase 4D isoform PDE4D5) c4(75384, 75385) c5(75386, 75387, 75388, 75389, 75390, 75391, 75392, 75393, 75394, 75395, 75396, 75397) c6( ) c7(bM, dD, eR, ea, aw, ht, cz, oR, aj, v, j, ct, nA, hz, eq, agJ, ad, dZ, bri, rS, M, EL, coV, eN, xa, eu, d, vs, pT, Kp, ek, EJ, pH, wg, ac, yl, xZ, cj, at, cS, aS, ei, coU); #c1(PDE4D) c2(NP_001184148) c3 (cAMP-specific 3′,5′-cyclic phosphodiesterase 4D isoform PDE408) c4(75398) c5(75399, 75400, 75401, 75402, 75403, 75404) c6( ) c7(bM, dD, eR, ea, aw, ht, cz, oR, aj, v, j, ct, nA, hz, eq, agJ, ad, dZ, bri, rS, M, EL, coV, eN, xa, eu, d, vs, pT, Kp, ek, EJ, pH, wg, ac, yl, xZ, cj, at, cS, aS, ei, coU); #c1(PDE4D) c2(NP_001184149) c3(cAMP-specific 3′,5′-cyclic phosphodiesterase 4D isoform PDE4D9) c4(75405) c5(75406, 75407, 75408, 75409, 75410, 75411) c6( ) c7(bM, dD, eR, ea, aw, ht, cz, oR, aj, v, j, ct, nA, hz, eq, agJ, ad, dZ, bri, rS, M, EL, coV, eN, xa, eu, d, vs, pT, Kp, ek, EJ, pH, wg, ac, yl, xZ, cj, at, cS, aS, ei, coU); #c1(PDE4D) c2(NP_001184150) c3(cAMP-specific 3′,5′-cyclic phosphodiesterase 4D isoform PDE4D2) c4( ) c5(75412, 75413, 75414, 75415, 75416, 75417, 75418, 75419, 75420, 75421) c6( ) c7(bM, dD, eR, ea, aw, ht, cz, oR, aj, v, j, ct, nA, hz, eq, agJ, ad, dZ, bri, rS, M, EL, coV, eN, xa, eu, d, vs, pT, Kp, ek, EJ, pH, wg, ac, yl, xZ, cj, at, cS, aS, ei, coU); #c1(PDE4D) c2(NP_001184151) c3(cAMP-specific 3′,5′-cyclic phosphodiesterase 4D isoform PDE4D1) c4( ) c5(75422) c6( ) c7(bM, dD, eR, ea, aw, ht, cz, oR, aj, v, j, ct, nA, hz, eq, agJ, ad, dZ, bri, rS, M, EL, coV, eN, xa, eu, d, vs, pT, Kp, ek, EJ, pH, wg, ac, yl, xZ, cj, at, cS, aS, ei, coU); #c1(PDE4D) c2(NP_00184152) c3(cAMP-specific 3′,5′-cyclic phosphodiesterase 4D isoform PDE4D6) c4(75423) c5(75424, 75425, 75426, 75427, 75428, 75429) c6( ) c7(bM, dD, eR, ea, aw, ht, cz, oR, aj, v, j, ct, nA, hz, eq, agJ, ad, dZ, bri, rS, M, EL, coV, eN, xa, eu, d, vs, pT, Kp, ek, EJ, pH, wg, ac, yl, xZ, cj, at, cS, aS, ei, coU); #c1(PDE4D) c2(NP_006194) c3(cAMP-specific 3′,5′-cyclic phosphodiesterase 4D isoform PDE4D3) c4(162779) c5(162780, 162781, 162782, 162783, 162784, 162785, 162786, 162787, 162788, 162789, 162790, 162791) c6( ) c7(bM, dD, eR, ea, aw, ht, cz, oR, aj, v, j, ct, nA, hz, eq, agJ, ad, dZ, bri, rS, M, EL, coV, eN, xa, eu, d, vs, pT, Kp, ek, EJ, pH, wg, ac, yl, xZ, cj, at, cS, aS, ei, coU); #c1(PDE4D) c2(XP_011541772) c3(cAMP-specific 3′,5′-cyclic phosphodiesterase 4D isoform X3) c4(340379) c5( ) c6( ) c7(bM, dD, eR, ea, aw, ht, cz, oR, aj, v, j, ct, nA, hz, eq, agJ, ad, dZ, bri, rS, M, EL, coV, eN, xa, eu, d, vs, pT, Kp, ek, EJ, pH, wg, ac, yl, xZ, cj, at, cS, aS, ei, coU); #c1(PDE4D) c2(XP_011541779) c3(cAMP-specific 3′,5′-cyclic phosphodiesterase 4D isoform X8) c4(340380) c5( ) c6( ) c7(bM, dD, eR, ea, aw, ht, cz, oR, aj, v, j, ct, nA, hz, eq, agJ, ad, dZ, bri, rS, M, EL, coV, eN, xa, eu, d, vs, pT, Kp, ek, EJ, pH, wg, ac, yl, xZ, cj, at, cS, aS, ei, coU); #c1(PDE4D) c2(XP_011541781) c3(cAMP-specific 3′,5′-cyclic phosphodiesterase 4D isoform X9) c4(340381) c5( ) c6( ) c7(bM, dD, eR, ea, aw, ht, cz, oR, aj, v, j, ct, nA, hz, eq, agJ, ad, dZ, bri, rS, M, EL, coV, eN, xa, eu, d, vs, pT, Kp, ek, EJ, pH, wg, ac, yl, xZ, cj, at, cS, aS, ei, coU); #c1(PDE5A) c2(NP_001074) c3(CGMP-specific 3′,5′-cyclic phosphodiesterase isoform 1) c4(37161) c5( ) c6( ) c7(lD, wH, qb, e, aT, aw, eX, sM, cR, aj, ah, ci, q, J, cd, BY, cc, aT, h, lG, cy, eV, ey, K, bq, wt, bc, wC, ly, Vx, aW, Y, r, uC, adA, lx, Kc, ac, wE, hc, ll, gZ, lC, X, ao, vQ); #c1(PDE5A) c2(NP_236914) c3(CGMP-specific 3′,5′-cyclic phosphodiesterase isoform 2) c4(228660) c5(228661, 228662, 228663, 228664, 228665, 228666, 228667) c6( ) c7(lD, wH, qb, e, al, aw, eX, sM, cR, aj, ah, ci, q, J, cd, BY, cc, aT, h, lG, cy, eV, ey, K, bq, wt, bc, wC, ly, Vx, aW, Y, r, uC, adA, lx, Kc, ac, wE, hc, ll, gZ, lC, X, ao, vQ); #c1(PDE5A) c2(NP_246273) c3 (cGMP-specific 3′,5′-cyclic phosphodiesterase isoform 3) c4(228668) c5( ) c6( ) c7(lD, wH, qb, e, al, aw, eX, sM, cR, aj, ah, ci, q, J, cd, BY, cc, aT, h, lG, cy, eV, ey, K, bq, wt, bc, wC, ly, Vx, aW, Y, r, uC, adA, lx, Kc, ac, wE, hc, ll, gZ, lC, X, ao, vQ); #c1(PDE6A) c2(NP_000431) c3(rod cGMP-specific 3′,5′-cyclic phosphodiesterase subunit alpha) c4(8909) c5(8910, 8911, 8912, 8913, 8914, 8915) c6( ) c7(coW, lR, nC); #c1(PDE6B) c2(NP_000274) c3(rod cGMP-specific 3′,5′-cyclic phosphodiesterase subunit beta isoform 1) c4(6071) c5( ) c6( ) c7(xX, lR, ig, aCk, cox, coY, nw, nk, nC, ayj, de); #c1(PDEGB) c2(NP_001138763) c3(rod cGMP-specific 3′,5′-cyclic phosphodiesterase subunit beta isoform 2) c4(57595) c5(57596, 57597, 57598, 57599, 57600) c6( ) c7(xX, lR, ig, aCk, cox, coY, nw, nk, nC, ayj, de); #c1(PDEGB) c2(XP_011511776) c3(rod cGMP-specific 3′,5′-cyclic phosphodiesterase subunit beta isoform X2) c4(302471) c5(302472, 302473, 302474, 302475, 302476, 302477, 302478, 302479, 302480, 302481, 302482, 302483, 302484) c6( ) c7(xX, lR, ig, aCk, cox, coY, nw, nk, nC, ayj, de); #c1(PDE6C) c2(NP_006195) c3(cone cGMP-specific 3′,5′-cyclic phosphodiesterase subunit alpha′) c4(162792) c5(162793, 162794, 162795, 162796, 162797, 162798) c6( ) c7(cpa, aQs, coZ, lR, nw, OL, nk, aQu, xa); #c1(PDE6D) c2(NP_002592) c3(retinal rod rhodopsin-sensitive cGMP 3′,5′-cyclic phosphodiesterase subunit delta isoform 1) c4(130113) c5(130114, 130115, 130116, 130117, 130118, 130119) c6( ) c7(LB, cpb, h); #c1(PDE6G) c2(NP_002593) c3(retinal rod rhodopsin-sensitive cGMP 3′,5′-cyclic phosphodiesterase subunit gamma) c4( ) c5(130120, 130121, 130122, 130123, 130124, 130125) c6( ) c7(LB, cpc, xX, nC); #c1(PDE6G) c2(XP_006722005) c3(retinal rod rhodopsin-sensitive cGMP 3′,5′-cyclic phosphodiesterase subunit gamma isoform X1) c4(294530, 294531) c5(294532, 294533, 294534, 294535, 294536, 294537, 294538) c6( ) c7(LB, cpc, xX, nC); #c1(PDE6H) c2(NP_006196) c3(retinal cone rhodopsin-sensitive cGMP 3′,5′-cyclic phosphodiesterase subunit gamma) c4(162799) c5(162800, 162801, 162802, 162803, 162804, 162805) c6( ) c7(cpd, cpe, OL); #c1(PDE7A) c2(NP_001229247) c3(high affinity cAMP-specific 3′,5′-cyclic phosphodiesterase 7A isoform c) c4(84031, 84032) c5(84033, 84034, 84035, 84036, 84037, 84038, 84039, 84040, 84041, 84042, 84043, 84044) c6( ) c7(yw, cj, at, fD, lC, ao); #c1(PDE7A) c2(XP_011515842) c3(high affinity cAMP-specific 3′,5′-cyclic phosphodiesterase 7A isoform X1) c4(308809) c5(308810, 308811, 308812, 308813, 308814, 308815) c6( ) c7(yw, cj, at, fD, lC, ao); #c1(PDE7B) c2(NP_061818) c3(cAMP-specific 3′,5′-cyclic phosphodiesterase 78) c4(200477) c5(200478, 200479, 200480, 200481, 200482, 200483, 200484, 200485, 200486, 200487, 200488, 200489) c6( ) c7(cj); #c1(PDE7B) c2(XP_005266988) c3(cAMP-specific 3′,5′-cyclic phosphodiesterase 78 isoform X1) c4(280596) c5(280597, 280598, 280599, 280600, 280601, 280602) c6( ) c7(cj); #c1(PDE7B) c2(XP_011534057) c3(cAMP-specific 3′,5′-cyclic phosphodiesterase 78 isoform X2) c4(330244) c5( ) c6( ) c7(cj); #c1(PDE8A) c2(NP_002596) c3(high affinity cAMP-specific and IBMX-insensitive 3′,5′-cyclic phosphodiesterase 8A isoform 1) c4(130126, 130127) c5(130128, 130129, 130130, 130131, 130132, 130133, 130134, 130135, 130136, 130137, 130138, 130139, 130140, 130141, 130142, 130143) c6( ) c7(h, aZ, kl, aw); #c1(PDE8A) c2(NP_775656) c3(high affinity cAMP-specific and IBMX-insensitive 3′,5′-cyclic phosphodiesterase 8A isoform 2) c4(250100) c5(250101, 250102, 250103, 250104, 250105, 250106) c6( ) c7(h, aZ, kl, aw); #c1(PDE8B) c2(NP_001025022) c3(high affinity cAMP-specific and IBMX-insensitive 3′,5′-cyclic phosphodiesterase 8B isoform 3) c4(29204) c5(29205, 29206, 29207, 29208, 29209, 29210) c6( ) c7(cpg, avH, s, gT, vf, aw, qQ, U, bcD, iw, hz, v, Hu, cph, xw, biO, af, cpf); #c1(PDE8B) c2(NP_001025023) c3(high affinity cAMP-specific and IBMX-insensitive 3′,5′-cyclic phosphodiesterase 88 isoform 5) c4(29211) c5(29212, 29213, 29214, 29215, 29216, 29217) c6( ) c7(cpg, avH, s, gT, vf, aw, qQ, U, bcD, iw, hz, v, Hu, cph, xw, biO, af, cpf); #c1(PDE8B) c2(NP_001025024) c3(high affinity cAMP-specific and IBMX-insensitive 3′,5′-cyclic phosphodiesterase 88 isoform 4) c4(29218) c5(29219, 29220, 29221, 29222, 29223, 29224) c6( ) c7(cpg, avH, s, gT, vf, aw, qQ, U, bcD, iw, hz, v, Hu, cph, xw, biO, af, cpf); #c1(PDE8B) c2(NP_001025025) c3(high affinity cAMP-specific and IBMX-insensitive 3′,5′-cyclic phosphodiesterase 8B isoform 2) c4(29225) c5(29226, 29227, 29228, 29229, 29230, 29231) c6( ) c7(cpg, avH, s, gT, vf, aw, qQ, U, bcD, iw, hz, v, Hu, cph, xw, biO, af, cpf); #c1(PDE8B) c2(NP_003710) c3(high affinity cAMP-specific and IBMX-insensitive 3′,5′-cyclic phosphodiesterase 8B isoform 1) c4(140344) c5(140345, 140346, 140347, 140348, 140349, 140350) c6( ) c7(cpg, avH, s, gT, vf, aw, qQ, U, bcD, iw, hz, v, Hu, cph, xw, biO, af, cpf); #c1(PDE8B) c2(XP_006714789) c3(high affinity cAMP-specific and IBMX-insensitive 3′,5′-cyclic phosphodiesterase 88 isoform X2) c4( ) c5(288159) c6( ) c7(cpg, avH, s, gT, vf, aw, qQ, U, bcD, iw, hz, v, Hu, cph, xw, biO, af, cpf); #c1(PDE8B) c2(XP_011542002) c3(high affinity cAMP-specific and IBMX-insensitive 3′,5′-cyclic phosphodiesterase 8B isoform X5) c4(340498) c5( ) c6( ) c7(cpg, avH, s, gT, vf, aw, qQ, U, bcD, iw, hz, v, Hu, cph, xw, biO, af, cpf); #c1(PDE8B) c2(XP_011542006) c3(high affinity cAMP-specific and IBMX-insensitive 3′,5′-cyclic phosphodiesterase 8B isoform X11) c4( ) c5(340499, 340500, 340501, 340502, 340503, 340504) c6( ) c7(cpg, avH, s, gT, vf, aw, qQ, U, bcD, iw, hz, v, Hu, cph, xw, biO, af, cpf); #c1(PDE9A) c2(NP_001001567) c3(high affinity cGMP-specific 3′,5′-cyclic phosphodiesterase 9A isoform b) c4(18799, 18800) c5(18801, 18802, 18803, 18804, 18805, 18806, 18807, 18808, 18809, 18810, 18811, 18812) c6( ) c7(Z, bM, f, U, bO); #c1(PDE9A) c2(NP_001001568) c3(high affinity cGMP-specific 3′,5′-cyclic phosphodiesterase 9A isoform c) c4(18813) c5(18814, 18815, 18816, 18817, 18818, 18819) c6( ) c7(Z, bM, f, U, bO); #c1(PDE9A) c2(NP_001001569) c3(high affinity cGMP-specific 3′,5′-cyclic phosphodiesterase 9A isoform d) c4(18820) c5(18821, 18822, 18823, 18824, 18825, 18826) c6( ) c7(Z, bM, f, U, bO); #c1(PDE5A) c2(NP_001001570) c3(high affinity cGMP-specific 3′,5′-cyclic phosphodiesterase 9A isoform e) c4(18827) c5(18828, 18829, 18830, 18831, 18832, 18833) c6( ) c7(Z, bM, f, U, bO); #c1(PDE5A) c2(NP_001001571) c3(high affinity cGMP-specific 3′,5′-cyclic phosphodiesterase 9A isoform f) c4(18834) c5(18835, 18836, 18837, 18838, 18839, 18840) c6( ) c7(Z, bM, f, U, bO); #c1(PDE5A) c2(NP_001001574) c3(high affinity cGMP-specific 3′,5′-cyclic phosphodiesterase 9A isoform h) c4(18841) c5(18842, 18843, 18844, 18845, 18846, 18847) c6( ) c7(Z, bM, f, U, bO); #c1(PDE5A) c2(NP_001001575) c3(high affinity CGMP-specific 3′,5′-cyclic phosphodiesterase 9A isoform i) c4(18848) c5(18849, 18850, 18851, 18852, 18853, 18854) c6( ) c7(Z, bM, f, U, bO); #c1(PDE9A) c2(NP_001001577) c3(high affinity cGMP-specific 3′,5′-cyclic phosphodiesterase 9A isoform k) c4(18855) c5(18856, 18857, 18858, 18859, 18860, 18861) c6( ) c7(Z, bM, f, U, bO); #c1(PDE9A) c2(NP_001001578) c3(high affinity CGMP-specific 3′,5′-cyclic phosphodiesterase 9A isoform 1) c4(18862) c5(18863, 18864, 18865, 18866, 18867, 18868) c6( ) c7(Z, bM, f, U, bO); #c1(PDE5A) c2(NP_001001581) c3(high affinity cGMP-specific 3′,5′-cyclic phosphodiesterase 9A isoform m) c4(18869) c5(18870, 18871, 18872, 18873, 18874, 18875) c6( ) c7(Z, bM, f, U, bO); #c1(PDE9A) c2(NP_001001582) c3(high affinity cGMP-specific 3′,5′-cyclic phosphodiesterase 9A isoform n) c4(18876) c5(18877, 18878, 18879, 18880, 18881, 18882) c6( ) c7(Z, bM, f, U, bO); #c1(PDE9A) c2(NP_001001583) c3(high affinity cGMP-specific 3′,5′-cyclic phosphodiesterase 9A isoform o) c4(18883) c5(18884, 18885, 18886, 18887, 18888, 18889) c6( ) c7(Z, bM, f, U, bO); #c1(PDE9A) c2(NP_002597) c3(high affinity cGMP-specific 3′,5′-cyclic phosphodiesterase 9A isoform a) c4(130144) c5(130145, 130146, 130147, 130148, 130149, 130150) c6( ) c7(Z, bM, f, U, bO); #c1(PDF) c2(NP_071736) c3(peptide deformylase, mitochondrial precursor) c4(211858) c5(211859, 211860, 211861, 211862, 211863, 211864) c6( ) c7(ad); #c1(PDGFA) c2(NP_002598) c3(platelet-derived growth factor subunit A isoform 1 preproprotein) c4( ) c5(130151, 130152, 130153, 130154, 130155, 130156, 130157) c6(130158, 130159) c7(lD, bM, cZ, T, dD, aC, On, ea, fG, aw, jh, n, j, ci, eq, PJ, lR, zP, Fc, fr, eA, er, cpi, BY, dP, ip, afb, hd, ib, aHu, jz, Ru, bW, aeO, rC, M, aT, hP, zg, aDC, dd, ey, nJ, vP, dl, Yi, aa, uQ, yo, zQ, d, aeR, Y, ff, aF, yq, en, g, as, eF, bz, pb, LB, ar, D, sS, bF, pc, bQU, m, lC, hS); #c1(PDGFA) c2(NP_148983) c3(platelet-derived growth factor subunit A isoform 2 preproprotein) c4( ) c5(226704, 226705, 226706, 226707, 226708, 226709, 226710, 226711, 226712, 226713, 226714, 226715, 226716) c6(226717, 226718, 226719) c7(lD, bM, cZ, T, dD, aC, On, ea, fG, aw, jh, n, j, ci, eq, PJ, lR, zP, Fc, fr, eA, er, cpi, BY, dP, ip, afb, hd, ib, alu, jz, Ru, bW, aeD, rC, M, aT, hP, zg, aDC, dd, ey, nJ, vP, dl, Yi, aa, uQ, yo, zQ, d, aeR, Y, ff, aF, yq, en, g, as, eF, bz, pb, LB, ar, D, sS, bF, pc, bQU, m, lC, hS); #c1(PDGFA) c2(XP_011513718) c3(platelet-derived growth factor subunit A isoform X2) c4(306003, 306004) c5(306005, 306006) c6( ) c7(lD, bM, cZ, T, dD, aC, On, ea, fG, aw, jh, n, j, ci, eq, PJ, lR, zP, Fc, fr, eA, er, cpi, BY, dP, ip, afb, hd, ib, aHu, jz, Ru, bW, aeO, rC, M, aT, hP, zg, aDC, dd, ey, nJ, vP, dl, Yi, aa, uQ, yo, zQ, d, aeR, Y, ff, aF, yq, en, g, as, eF, bz, pb, LB, ar, D, sS, bF, pc, bQU, m, lC, hS); #c1(PDGFA) c2(XP_011513720) c3(platelet-derived growth factor subunit A isoform X4) c4(306007) c5(306008, 306009, 306010) c6( ) c7(lD, bM, cZ, T, dD, aC, On, ea, fG, aw, jh, n, j, ci, eq, PJ, lR, zP, Fc, fr, eA, er, cpi, BY, dP, ip, afb, hd, ib, aHu, jz, Ru, bW, aeO, rC, M, aT, hP, zg, aDC, dd, ey, nJ, vP, dl, Yi, aa, uQ, yo, zQ, d, aeR, Y, ff, aF, yq, en, g, as, eF, bz, pb, LB, ar, D, sS, bF, pc, bQU, m, lC, hS); #c1(PDGFB) c2(NP_002599) c3(platelet-derived growth factor subunit B isoform 1 preproprotein) c4(130160) c5(130161, 130162, 130163, 130164, 130165, 130166, 130167, 130168, 130169, 130170, 130171) c6(130172, 130173) c7(bM, sY, T, dD, aC, fR, hV, ea, fG, al, bXc, aYc, cpk, kv, eq, ci, uE, yV, eA, ip, bOV, av, ib, aEX, oW, aT, cpj, aaj, h, iY, ey, bq, bc, jf, eG, d, lR, iU, aW, Y, eH, as, eF, t, uC, bz, kJ, pb, LB, ck, D, ar, bF, pc, c, nB, dB, hk, hc, cpl, aGo, lf, bQU, iT, pg, aRU, hh, lC, bt, B, hS); #c1(PDGFB) c2(NP_148937) c3(platelet-derived growth factor subunit B isoform 2 preproprotein) c4(226636, 226637, 226638) c5(226639, 226640, 226641, 226642, 226643, 226644, 226645, 226646, 226647, 226648, 226649, 226650, 226651, 226652, 226653, 226654, 226655, 226656, 226657, 226658, 226659, 226660, 226661) c6(226662, 226663, 226664, 226665, 226666, 226667) c7(bM, sY, T, dD, aC, fR, hV, ea, fG, al, bXc, aYc, cpk, kv, eq, ci, uE, yV, eA, ip, bOV, av, ib, aEX, oW, aT, cpj, aaj, h, iY, ey, bq, bc, jf, eG, d, lR, iU, aW, Y, eH, as, eF, t, uC, bz, kJ, pb, LB, ck, D, ar, bF, pc, c, nB, dB, hk, hc, cpl, aGo, lf, bQU, iT, pg, aRU, hh, lC, bt, B, hS); #c1(PDGFC) c2(NP_057289) c3(platelet-derived growth factor C precursor) c4(189884, 189885, 189886, 189887, 189888, 189889) c5(189890, 189891, 189892, 189893, 189894, 189895, 189896, 189897, 189898, 189899, 189900, 189901, 189902, 189903, 189904, 189905, 189906) c6(189907, 189908, 189909, 189910) c7(sY, aoU, T, dD, dK, hy, eq, fr, Na, bv, av, hd, ib, Db, aT, Nc, ey, Xh, BYL, bxg, jf, B, BN, aW, Y, oV, uC, apM, bE, hc, ne); #c1(PDGFC) c2(XP_011530426) c3(platelet-derived growth factor C isoform X1) c4(326272) c5( ) c6( ) c7(sY, aoU, T, dD, dK, hy, eq, fr, Na, bv, av, hd, ib, Db, aT, Nc, ey, Xh, BYL, bxg, jf, B, BN, aW, Y, oV, uC, apM, bE, hc, ne); #c1(PDGFC) c2(XP_011530427) c3(platelet-derived growth factor C isoform X2) c4( ) c5(326273, 326274, 326275, 326276, 326277, 326278, 326279, 326280, 326281, 326282, 326283, 326284, 326285, 326286, 326287, 326288) c6(326289, 326290, 326291, 326292) c7(sY, aoU, T, dD, dK, hy, eq, fr, Na, bv, av, hd, ib, Db, aT, Nc, ey, Xh, BYL, bxg, jf, B, BN, aW, Y, oV, uC, apM, bE, hc, ne); #c1(PDGFD) c2(NP_079484) c3(platelet-derived growth factor D isoform 1 precursor) c4(218305) c5(218306, 218307, 218308, 218309, 218310, 218311, 218312, 218313, 218314) c6(218315, 218316) c7(T, dD, aC, dA, ea, Lx, cz, aj, j, ci, eq, fr, ad, av, jz, ib, sN, yg, aT, vw, K, ae, eu, jf, aW, oV, ac, hc, dy, sH, hS); #c1(PDGFD) c2(NP_149126) c3(platelet-derived growth factor D isoform 2 precursor) c4(227330) c5(227331, 227332, 227333, 227334, 227335, 227336, 227337, 227338, 227339) c6(227340, 227341) c7(T, dD, aC, dA, ea, Lx, cz, aj, j, ci, eq, fr, ad, av, jz, ib, sN, yg, aT, vw, K, ae, eu, jf, aW, oV, ac, hc, dy, sH, hS); #c1(PDGFRA) c2(XP_006714102) c3(platelet-derived growth factor receptor alpha isoform X2) c4( ) c5(287547) c6( ) c7(Fv, bM, T, aC, fR, if, qj, ci, Qp, ip, x, bv, Na, aE, pX, aeO, eL, cp, cpm, Qe, cpn, aa, aeR, cpp, aW, Az, hl, uY, Qm, g, as, eF, Fu, Qt, zV, ix, eM, iT, lp, vA, oy, cj, apy, uJ, k, m, Xu, Th, aiu, dD, sw, qy, n, oY, ft, jz, hw, Lv, eV, hn, dL, bkN, Nr, Tc, bFc, AQ, aq, po, op, yR, Qf, dB, eD, wk, bQU, apY, B, aoU, dA, ex, fZ, aA, j, q, eA, aT, dz, bK, Tj, ae, af, dl, jf, iU, Y, ayB, r, hf, t, iK, pb, ck, Ta, nM, ac, gX, bt, On, bkL, jh, eq, hF, Fc, ad, afb, dP, av, iC, cpq, cpo, hP, fb, d, lx, et, vj, eH, aeV, aF, aiz, iR, aTb, lU, Nq, Ew, akC, y, ne, jr, pC); #c1(PDGFRA) c2(XP_006714104) c3(platelet-derived growth factor receptor alpha isoform X3) c4( ) c5(287548, 287549, 287550, 287551, 287552, 287553, 287554, 287555, 287556, 287557, 287558, 287559, 287560, 287561, 287562, 287563, 287564, 287565, 287566, 287567, 287568, 287569, 287570, 287571) c6( ) c7(Fv, bM, T, aC, fR, if, qj, ci, Qp, ip, x, bv, Na, aE, pX, aeO, eL, cp, cpm, Qe, cpn, aa, aeR, cpp, aW, Az, hl, uY, Qm, g, as, eF, Fu, Qt, zV, ix, eM, iT, lp, vA, oy, cj, apy, uJ, k, m, Xu, Th, aiu, dD, sw, qy, n, oY, ft, jz, hw, Lv, eV, hn, dL, bkN, Nr, Tc, bFc, AQ, aq, po, op, yR, Of, dB, eD, wk, bQU, apY, B, aoU, dA, eX, fZ, aA, j, q, eA, aT, dz, bK, Tj, ae, af, dl, jf, iU, Y, ayB, r, hf, t, iK, pb, ck, Ta, nM, ac, gX, bt, On, bkL, jh, eq, hF, Fc, ad, afb, dP, av, iC, cpq, cpo, hP, fb, d, lx, et, vj, eH, aeV, aF, aiz, iR, aTb, lU, Nq, Ew, akC, y, ne, jr, pC); #c1(PDGFRA) c2(XP_011532688) c3(platelet-derived growth factor receptor alpha isoform X1) c4(328722, 328723, 328724, 328725, 328726) c5(328727, 328728, 328729, 328730, 328731) c6( ) c7(Fv, bM, T, aC, FR, if, qj, ci, Qp, ip, x, bv, Na, aE, pX, aeO, eL, cp, cpm, Qe, cpn, aa, aeR, cpp, aW, Az, hl, uY, Qm, g, as, eF, Fu, Qt, zV, ix, eM, iT, lp, vA, oy, cj, apy, uJ, k, m, Xu, Th, aiu, dD, sw, qy, n, oY, ft, jz, hw, Lv, eV, hn, dL, bkN, Nr, Tc, bFc, AQ, aq, po, op, yR, Qf, dB, eD, wk, bQU, apY, B, aol, dA, eX, fZ, aA, j, q, eA, aT, dz, bK, Tj, ae, af, dl, jf, iU, Y, ayB, r, hf, t, iK, pb, ck, Ta, nM, ac, gX, bt, On, bkL, jh, eq, hF, Fc, ad, afb, dP, av, iC, cpq, cpo, hP, fb, d, lx, et, vj, eH, aeV, aF, aiz, iR, aTb, lU, Nq, Ew, akC, y, ne, jr, pC); #c1(PDGFRB) c2(NP_002600) c3(platelet-derived growth factor receptor beta precursor) c4(130174, 130175) c5(130176, 130177, 130178, 130179, 130180, 130181, 130182, 130183, 130184, 130185, 130186, 130187) c6( ) c7(bM, T, pf, yS, cps, qj, ci, lR, ip, bv, aE, aeO, cda, K, eL, eG, oP, aeR, pr, aW, Ls, as, eF, cm, Qt, eM, iT, oy, cj, uJ, m, Th, ik, aiu, cpr, sw, NT, qy, fr, bd, cld, cpt, Xe, Lv, aaj, dL, cY, Nr, aJ, fQ, aq, po, pm, bkn, asl, dB, bQU, B, iH, cz, q, er, eA, aT, dz, bq, ae, af, dl, aZF, jf, VC, iU, vT, nO, Y, aBN, r, bu, t, pb, ck, nM, kP, bKf, hc, bt, hl, jh, iJ, ad, bkq, NS, hP, fb, nJ, pq, d, et, vj, eH, kD, aF, iV, iR, adj, bz, Nq, LB, D, es, akC, lC, jr, pC); #c1(PDGFRB) c2(XP_011535962) c3(platelet-derived growth factor receptor beta isoform X2) c4(333241) c5(333242, 333243, 333244, 333245, 333246, 333247) c6( ) c7(bM, T, pf, yS, cps, qj, ci, lR, ip, bv, aE, aeO, cda, K, eL, eG, oP, aeR, pr, aW, Ls, as, eF, cm, Qt, eM, iT, oy, cj, uJ, m, Th, ik, aiu, cpr, sw, NT, qy, fr, bd, cld, cpt, Xe, Lv, aaj, dL, cY, Nr, aJ, fQ, aq, po, pm, bkn, asl, dB, bQU, B, iH, cz, q, er, eA, aT, dz, bq, ae, af, dl, aZF, jf, VC, iU, vT, nO, Y, aBN, r, bu, t, pb, ck, nM, kP, bKf, hc, bt, hl, jh, iJ, ad, bkq, NS, hP, fb, nJ, pq, d, et, vj, eH, kD, aF, iV, iR, adj, bz, Nq, LB, D, es, akC, lC, jr, pC); #c1(PDGFRL) c2(XP_011542860) c3(platelet-derived growth factor receptor-like protein isoform X1) c4(341459) c5(341460, 341461, 341462, 341463, 341464, 341465, 341466, 341467, 341468, 341469, 341470, 341471, 341472, 341473) c6( ) c7(bE, ff, r, hP, ci, en, g, hH, fq, B); #c1(PDHA1) c2(NP_000275) c3(pyruvate dehydrogenase E1 component subunit alpha, somatic form, mitochondrial isoform 1 precursor) c4(6072, 6073) c5(6074, 6075, 6076, 6077, 6078, 6079, 6080, 6081, 6082, 6083, 6084, 6085) c6( ) c7(SE, ajU, aA, aU, n, cpu, j, aNH, ad, alk); #c1(PDHA1) c2(NP_001166925) c3(pyruvate dehydrogenase E1 component subunit alpha, somatic form, mitochondrial isoform 2 precursor) c4(68597, 68598) c5(68599, 68600, 68601, 68602, 68603, 68604, 68605, 68606, 68607, 68608, 68609, 68610, 68611) c6( ) c7(SE, ajU, aA, aU, n, cpu, j, aNH, ad, alk); #c1(PDHA1) c2(NP_001166926) c3(pyruvate dehydrogenase E1 component subunit alpha, somatic form, mitochondrial isoform 3 precursor) c4(68612, 68613, 68614) c5(68615, 68616, 68617, 68618, 68619, 68620, 68621, 68622, 68623, 68624, 68625, 68626) c6( ) c7(SE, ajU, aA, aU, n, cpu, j, aNH, ad, alk); #c1(PDHA1) c2(NP_001166927) c3 (pyruvate dehydrogenase E1 component subunit alpha, somatic form, mitochondrial isoform 4 precursor) c4(68627) c5(68628, 68629, 68630, 68631, 68632, 68633, 68634, 68635, 68636, 68637, 68638, 68639, 68640, 68641, 68642) c6( ) c7(SE, ajU, aA, aU, n, cpu, j, aNH, ad, alk); #c1(PDHA1) c2(XP_011543833) c3(pyruvate dehydrogenase E1 component subunit alpha, somatic form, mitochondrial isoform X1) c4(342970) c5( ) c6( ) c7(SE, ajU, aA, aU, n, cpu, j, aNH, ad, alk); #c1(PDHA1) c2(XP_011543834) c3(pyruvate dehydrogenase E1 component subunit alpha, somatic form, mitochondrial isoform X2) c4( ) c5(342971, 342972, 342973, 342974, 342975, 342976, 342977, 342978, 342979) c6( ) c7(SE, ajU, aA, aU, n, cpu, j, aNH, ad, alk); #c1(PDHB) c2(NP_000916) c3(pyruvate dehydrogenase E1 component subunit beta, mitochondrial isoform 1 precursor) c4(17163) c5(17164, 17165, 17166, 17167, 17168, (7169) c6( ) c7(fr, cpv, aNH); #c1(PDHB) c2(NP_001166939) c3 (pyruvate dehydrogenase E1 component subunit beta, mitochondrial isoform 2 precursor) c4(68686) c5(68687, 68688, 68689, 68690, 68691, 68692) c6( ) c7(fr, cpv, aNH); #c1(PDHX) c2(NP_001128496) c3(pyruvate dehydrogenase protein X component, mitochondrial isoform 2) c4(50100) c5(50101, 50102, 50103, 50104, 50105, 50106) c6( ) c7(bL, T, aw, aA, aj, dG, ci, nA, aNH, eJ, J, mj, mh, aE, hb, ey, K, aKG, bc, pl, eG, A, cpw, eZ, kp, aF, LV, aam, mf, m, ei); #c1(PDHX) c2(NP_001159630) c3(pyruvate dehydrogenase protein X component, mitochondrial isoform 3 precursor) c4(64476) c5(64477, 64478, 64479, 64480, 64481, 64482) c6( ) c7(bL, T, aw, aA, aj, dG, ci, nA, aNH, eJ, J, mj, mh, aE, hb, ey, K, aKG, bc, pl, eG, A, cpw, eZ, kp, af, LV, aam, mf, m, ei); #c1(PDHX) c2(NP_003468) c3(pyruvate dehydrogenase protein X component, mitochondrial isoform 1 precursor) c4(138582) c5(138583, 138584, 138585, 138586, 138587, 138588) c6( ) c7(bL, T, aw, aA, aj, dG, ci, nA, aNH, eJ, J, mj, mh, aE, hb, ey, K, aKG, bc, pl, eG, A, cpw, eZ, kp, aF, LV, aam, mf, m, ei); #c1(PDIA2) c2(NP_006840) c3(protein disulfide-isomerase A2 precursor) c4(168552, 168553, 168554) c5(168555, 168556, 168557, 168558, 168559, 168560, 168561, 168562, 168563, 168564, 168565, 168566, 168567, 168568, 168569, 168570, 168571, 168572) c6( ) c7(amF, bL, aW, aT, aj, si, bD, cm, B, J); #c1(PDIA3) c2(NP_005304) c3(protein disulfide-isomerase A3 precursor) c4(154807, 154808, 154809, 154810) c5(154811, 154812, 154813, 154814, 154815, 154816, 154817, 154818, 154819, 154820, 154821, 154822, 154823, 154824, 154825, 154826, 154827, 154828, 154829, 154830, 154831, 154832, 154833, 154834) c6( ) c7(T, dt, gU, if, aT, bO, aA, ajp, n, qN, ad, dP, fU, akN, aT, K, ae, aW, Y, ff, kD, en, g, t, bw, DF, gk, cA, Hb, m, bt, B, vi); #c1(PDIA4) c2(NP_004902) c3(protein disulfide-isomerase A4 precursor) c4(150656) c5(150657, 150658, 150659, 150660, 150661, 150662) c6( ) c7(fe); #c1(PDIA5) c2(NP_006801) c3(protein disulfide-isomerase A5 precursor) c4(168199) c5(168200, 168201, 168202, 168203, 168204, 168205) c6( ) c7(dH); #c1(PDIA6) c2(NP_001269633) c3(protein disulfide-isomerase AG isoform a) c4( ) c5(102314, 102315, 102316, 102317, 102318) c6( ) c7(T, aW, eX, aT, eV, B, bA); #c1(PDIA6) c2(NP_001269634) c3(protein disulfide-isomerase A6 isoform b) c4(102319) c5(102320) c6( ) c7(T, aW, eX, aT, eV, B, bA); #c1(PDIA6) c2(NP_001269635) c3(protein disulfide-isomerase A6 isoform c) c4( ) c5(102321, 102322, 102323, 102324, 102325) c6( ) c7(T, aW, ex, aT, eV, B, bA); #c1(PDIA6) c2(NP_001269636) c3(protein disulfide-isomerase A6 isoform e precursor) c4(102326) c5(102327, 102328, 102329, 102330, 102331, 102332, 102333) c6( ) c7(T, aW, ex, aT, eV, B, bA); #c1(PDIA6) c2(NP_005733) c3(protein disulfide-isomerase AG isoform d precursor) c4(158947) c5(158948, 158949, 158950, 158951, 158952) c6( ) c7(T, aW, eX, aT, eV, B, bA); #c1(PDK1) c2(NP_001265478) c3([Pyruvate dehydrogenase (acetyl-transferring)] kinase isozyme 1, mitochondrial isoform 1 precursor) c4(98667) c5(98668, 98669, 98670, 98671, 98672, 98673) c6( ) c7(bL, T, ht, NT, aA, fd, bS, v, ci, Fb, fr, cd, q, ad, hd, bpj, NS, aT, h, hu, ask, sR, ae, aW, Y, r, aF, bz, ck, Qi, ac, nB, mx, bt, X); #c1(PDK1) c2(NP_002601) c3([Pyruvate dehydrogenase (acetyl-transferring)] kinase isozyme 1, mitochondrial isoform 2 precursor) c4(130188, 130189, 130190) c5(130191, 130192, 130193, 130194, 130195, 130196, 130197, 130198, 130199, 130200, 130201) c6( ) c7(bL, T, ht, NT, aA, fd, bS, v, ci, Fb, fr, cd, q, ad, hd, bpj, NS, aT, h, hu, ask, sR, ae, aW, Y, r, aF, bz, ck, Qi, ac, nB, mx, bt, X); #c1(PDK1) c2(XP_011509647) c3(pyruvate dehydrogenase (acetyl-transferring) kinase isozyme 1, mitochondrial isoform X2) c4( ) c5(300217, 300218, 300219, 300220, 300221, 300222, 300223, 300224, 300225, 300226, 300227) c6( ) c7(bL, T, ht, NT, aA, fd, bS, v, ci, Fb, fr, cd, q, ad, hd, bpj, NS, aT, h, hu, ask, sR, ae, aW, Y, r, aF, bz, ck, Qi, ac, nB, mx, bt, X); #c1(PDK2) c2(NP_001186828) c3(pyruvate dehydrogenase kinase, isozyme 2 isoform 2) c4( ) c5(78693, 78694, 78695, 78696, 78697, 78698, 78699) c6(78700, 78701) c7(bL, T, v, cc); #c1(PDK2) c2(NP_002602) c3(pyruvate dehydrogenase kinase, isozyme 2 isoform 1 precursor) c4(130202, 130203) c5(130204, 130205, 130206, 130207, 130208, 130209, 130210) c6( ) c7(bL, T, v, cc); #c1(PDK3) c2(NP_001135858) c3(pyruvate dehydrogenase kinase, isozyme 3 isoform 1 precursor) c4(53420) c5(53421, 53422, 53423, 53424, 53425, 53426) c6( ) c7(bX, cpx, cp, bU); #c1(PDK3) c2(NP_005382) c3(pyruvate dehydrogenase kinase, isozyme 3 isoform 2 precursor) c4(155582) c5(155583, 155584, 155585, 155586, 155587, 155588) c6( ) c7(bX, cpx, cp, bU); #c1(PDK4) c2(NP_002603) c3(pyruvate dehydrogenase kinase, isozyme 4) c4(130211, 130212) c5(130213, 130214, 130215, 130216, 130217, 130218, 130219, 130220, 130221, 130222, 130223, 130224, 130225, 130226) c6( ) c7(bL, cc, B, aq, v, j, K, ei, bq, cd, bc); #c1(PDL1M1) c2(NP_066272) c3(PDZ and LIM domain protein 1) c4(206884) c5(206885, 206886, 206887, 206888, 206889, 206890) c6( ) c7(alC, oh, af, K, azj); #c1(PDLIM2) c2(NP_067643) c3(PDZ and LIM domain protein 2 isoform 2) c4(209/76, 209177) c5(209178, 209179, 209180, 209181, 209182, 209183, 209184, 209185, 209186, 209187, 209188, 209189, 209190, 209191, 209192, 209193, 209194, 209195, 209196, 209197, 209198, 209199, 209200, 209201) c6( ) c7(T, iM, je, dK, r, vw, K, q); #c1(PDLIM2) c2(NP_789847) c3(PDZ and LIM domain protein 2 isoform 1) c4(252408) c5(252409, 252410, 252411, 252412, 252413, 252414, 252415) c6( ) c7(T, iM, je, dK, r, vw, K, q); #c1(PDLIM2) c2(NP_932159) c3(PDZ and LIM domain protein 2 isoform 3) c4(258834, 258835) c5(258836, 258837, 258838, 258839, 258840, 258841, 258842, 258843, 258844, 258845, 258846, 258847, 258848) c6( ) c7(T, iM, je, dK, r, vw, K, q); #c1(PDLIM3) c2(NP_001107579) c3(PDZ and LIM domain protein 3 isoform b) c4(42882) c5(42883, 42884, 42885, 42886, 42887, 42888) c6( ) c7(fd, bB, Tl, h, nJ, bc, vT, HY, TH, eH, aq, mo, eF, TJ, bE, TG, au, mx, gZ, rx, y, B); #c1(PDLIM3) c2(NP_055291) c3(PDZ and LIM domain protein 3 isoform a) c4(180321) c5(180322, 180323, 180324, 180325, 180326, 180327, 180328) c6( ) c7(fd, bB, Tl, h, nJ, bc, vT, HY, TH, eH, aq, mo, eF, TJ, bE, TG, au, mx, gZ, rx, y, B); #c1(PDLIM3) c2(XP_011530176) c3(PDZ and LIM domain protein 3 isoform X1) c4(326051) c5(326052, 326053, 326054, 326055, 326056) c6( ) c7(fd, bB, Tl, h, nJ, bc, vT, HY, TH, eH, aq, mo, eF, TJ, bE, TG, au, mx, gZ, rx, y, B); #c1(PDLIM4) c2(NP_001124499) c3(PDZ and LIM domain protein 4 isoform 2) c4(49178) c5(49179, 49180, 49181, 49182, 49183, 49184, 49185) c6( ) c7(Dw, T, kl, kP, bE, aW, aT, bB, n, Ds, aq, it, K, bw, bt, ae, B, ad, bA); #c1(PDLIM4) c2(NP_003678) c3(PDZ and LIM domain protein 4 isoform 1) c4(140126) c5(140127, 140128, 140129, 140130, 140131, 140132) c6( ) c7(Dw, T, kl, kP, bE, aW, aT, bB, n, Ds, aq, it, K, bw, bt, ae, B, ad, bA); #c1(PDLIM5) c2(NP_001011513) c3(PDZ and LIM domain protein 5 isoform b) c4(24481) c5(24482, 24483, 24484, 24485, 24486, 24487) c6( ) c7(bL, T, JZ, He, bO, bB, ti, v, j, aY, hF, pn, q, ad, hX, x, Tg, aE, lg, hb, oj, kH, aT, oW, vc, h, bK, K, oi, he, ae, je, iU, aW, yL, r, lo, aXL, Z, cm, bw, ck, ou, alw, bb, alC, iM, yW, bE, Ew, mx, hj, vL, hE, bt, B, us); #c1(PDLIM5) c2(NP_001011515) c3(PDZ and LIM domain protein 5 isoform d) c4(24488, 24489) c5(24490, 24491, 24492, 24493, 24494, 24495, 24496, 24497, 24498, 24499, 24500, 24501) c6( ) c7(bL, T, JZ, He, bO, bB, ti, v, j, aY, hF, pn, q, ad, hX, x, Tg, aE, lg, hb, oj, kH, aT, oW, vc, h, bK, K, oi, he, ae, je, iU, aW, yL, r, lo, aXL, Z, cm, bw, ck, ou, alw, bb, alC, iM, yW, bE, Ew, mx, hj, vL, hE, bt, B, us); #c1(PDLIM5) c2(NP_001011516) c3(PDZ and LIM domain protein 5 isoform e) c4(24502, 24503) c5(24504, 24505, 24506, 24507, 24508, 24509, 24510, 24511, 24512, 24513, 24514, 24515) c6( ) c7(bL, T, JZ, He, bO, bB, ti, v, j, aY, hF, pn, q, ad, hX, x, Tg, aE, lg, hb, oj, kH, aT, oW, vc, h, bk, K, oi, he, ae, je, iU, aW, yL, r, lo, aXL, Z, cm, bw, ck, ou, alw, bb, alC, iM, yW, bE, Ew, mx, hj, vL, hE, bt, B, us); #c1(PDLIM5) c2(NP_001243357) c3(PDZ and LIM domain protein 5 isoform h) c4( ) c5(90489, 90490, 90491, 90492, 90493) c6( ) c7(bL, T, JZ, He, bO, bB, ti, v, j, aY, hF, pn, q, ad, hX, x, Tg, aE, lg, hb, oj, kH, aT, oW, vc, h, bK, K, oi, he, ae, je, iU, aW, yL, r, lo, aXL, Z, cm, bw, ck, ou, alw, bb, alC, iM, yW, bE, Ew, mx, hj, vL, hE, bt, B, us); #c1(PDLIM5) c2(NP_001243358) c3(PDZ and LIM domain protein 5 isoform i) c4(90494, 90495, 90496, 90497, 90498, 90499) c5(90500, 90501, 90502, 90503, 90504, 90505, 90506, 90507, 90508, 90509, 90510, 90511, 90512, 90513, 90514, 90515, 90516, 90517, 90518, 90519, 90520, 90521, 90522, 90523, 90524, 90525, 90526, 90527, 90528, 90529, 90530, 90531, 90532, 90533, 90534, 90535, 90536, 90537, 90538, 90539, 90540, 90541, 90542, 90543, 90544, 90545, 90546, 90547, 90548, 90549, 90550, 90551, 90552, 90553, 90554, 90555, 90556, 90557, 90558, 90559, 90560, 90561, 90562, 90563, 90564, 90565, 90566, 90567, 90568, 90569, 90570, 90571, 90572, 90573, 90574, 90575, 90576, 90577, 90578, 90579) c6( ) c7(bL, T, JZ, He, bO, bB, ti, v, j, aY, hF, pn, q, ad, hX, x, Tg, aE, lg, hb, oj, kH, aT, oW, vc, h, bK, K, oi, he, ae, je, iU, aW, yL, r, lo, aXL, Z, cm, bw, ck, ou, aLw, bb, alC, iM, yW, bE, Ew, mx, hj, vL, hE, bt, B, us); #c1(PDLIM5) c2(NP_006448) c3(PDZ and LIM domain protein 5 isoform a) c4(165316, 165317) c5(165318, 165319, 165320, 165321, 165322, 165323, 165324, 165325) c6( ) c7(bL, T, JZ, He, bO, bB, ti, v, j, aY, hF, pn, q, ad, hX, x, Tg, aE, lg, hb, oj, kH, aT, oW, vc, h, bK, K, oi, he, ae, je, iU, aW, yL, r, lo, aXL, Z, cm, bw, ck, ou, alw, bb, alC, iM, yW, bE, Ew, mx, hj, vL, hE, bt, B, us); #c1(PDLIM7) c2(NP_005442) c3(PDZ and LIM domain protein 7 isoform 1) c4(156206, 156207) c5(156208, 156209, 156210, 156211, 156212, 156213) c6( ) c7(Mb, T, aEd, fM, Ke, dA, iW, ex, jh, aA, bB, n, ci, pz, ad, bkO, aJO, lg, aVq, ja, aT, h, eV, K, zg, amY, pk, ae, cpy, jr, hM, aW, oV, ff, Pg, kt, aq, lo, af, aoD, anW, en, g, lk, bz, XT, aJT, Cv, ck, aZx, iQ, Zg, bE, dc, cj, aEG, ciO, zv, bC, ij, it, B, KG); #c1(PDLIM7) c2(NP_976227) c3(PDZ and LIM domain protein 7 isoform 2) c4(263007) c5(263008, 263009, 263010, 263011, 263012, 263013) c6( ) c7(Mb, T, aEd, fM, Ke, dA, iW, ex, jh, aA, bB, n, ci, pz, ad, bkO, aJO, lg, aVq, ja, aT, h, eV, K, zg, amY, pk, ae, cpy, jr, hM, aW, oV, ff, Pg, kt, aq, lo, aF, aoD, anW, en, g, lk, bz, XT, aJT, Cv, ck, aZx, iQ, Zg, bE, dc, cj, aEG, ciO, zv, bC, ij, it, B, KG); #c1(PDLIM7) c2(NP_998801) c3(PDZ and LIM domain protein 7 isoform 4) c4(265995, 265996) c5(265997, 265998, 265999, 266000, 266001, 266002, 266003, 266004, 266005, 266006, 266007, 266008, 266009, 266010, 266011, 266012, 266013, 266014, 266015, 266016, 266017, 266018, 266019) c6( ) c7(Mb, T, aEd, fM, Ke, dA, iW, eX, jh, aA, bB, n, ci, pz, ad, bkO, aJO, lg, aVq, ja, aT, h, eV, K, zg, amY, pk, ae, cpy, jr, hM, aW, oV, ff, Pg, kt, aq, lo, aF, aoD, anW, en, g, lk, bz, XT, aJT, Cv, ck, aZx, iQ, Zg, bE, dc, cj, aEG, ciO, zv, bC, ij, it, B, KG); #c1(PDLIM7) c2(XP_011532999) c3(PDZ and LIM domain protein 7 isoform X2) c4(329237) c5( ) c6( ) c7(Mb, T, aEd, fM, Ke, dA, iW, eX, jh, aA, bB, n, ci, pz, ad, bkO, aJO, lg, aVq, ja, aT, h, eV, K, zg, amY, pk, ae, cpy, jr, hM, aW, oV, ff, Pg, kt, aq, lo, aF, aoD, anW, en, g, lk, bz, XT, aJT, Cv, ck, aZx, iQ, Zg, bE, dc, cj, aEG, ciO, zv, bC, ij, it, B, KG); #c1(PDLIM7) c2(XP_011533001) c3(PDZ and LIM domain protein 7 isoform X4) c4( ) c5(329238) c6( ) c7(Mb, T, aEd, fM, Ke, dA, iW, ex, jh, aA, bB, n, ci, pz, ad, bkO, aJO, lg, aVq, ja, aT, h, eV, K, zg, amY, pk, ae, cpy, jr, hM, aW, oV, ff, Pg, kt, aq, lo, aF, aoD, anW, en, g, lk, bz, XT, aJT, Cv, ck, aZx, iQ, Zg, bE, dc, cj, aEG, ciO, zv, bC, ij, it, B, KG); #c1(PDP1) c2(NP_001155251) c3(pyruvate dehyrogenase phosphatase catalytic subunit 1 isoform 2) c4(61555) c5(61556, 61557, 61558, 61559, 61560, 61561, 61562, 61563, 61564, 61565, 61566, 61567, 61568) c6( ) c7(ck, T, baE, SE, hb, Ni, h, bfN, aNH, cpz, ajU); #c1(PDP1) c2(XP_011515437) c3(pyruvate dehyrogenase phosphatase catalytic subunit 1 isoform X2) c4(308250, 308251, 308252) c5(308253) c6( ) c7(ck, T, baE, SE, hb, Ni, h, bfN, aNH, cpz, ajU); #c1(PDP1) c2(XP_011515439) c3(pyruvate dehyrogenase phosphatase catalytic subunit 1 isoform X1) c4(308254, 308255, 308256) c5(308257, 308258, 308259, 308260, 308261, 308262, 308263, 308264, 308265, 308266, 308267, 308268, 308269, 308270, 308271, 308272, 308273, 308274, 308275, 308276, 308277, 308278, 308279, 308280, 308281, 308282, 308283, 308284, 308285, 308286, 308287, 308288, 308289, 308290, 308291, 308292) c6( ) c7(ck, T, baE, SE, hb, Ni, h, bfN, aNH, cpz, ajU); #c1(PDP2) c2(NP_065837) c3([Pyruvate dehydrogenase [acetyl-transferring]-phosphatase 2, mitochondrial precursor) c4(205794) c5(205795, 205796, 205797, 205798, 205799, 205800) c6( ) c7(Y, wK); #c1(PDPK1) c2(NP_001248745) c3(3-phosphoinositide-dependent protein kinase 1 isoform 3) c4(93975, 93976) c5(93977, 93978, 93979, 93980, 93981, 93982, 93983, 93984, 93985, 93986, 93987, 93988) c6( ) c7(T, ht, NT, aA, bB, fd, bS, v, ci, Fb, fr, cd, q, ad, bv, hd, bpj, aT, h, hu, ask, ae, aW, aml, Y, r, Qq, kp, aF, g, bz, ck, Qi, nM, eM, nB, bE, mx, bt, B); #c1(PDPK1) c2(NP_002604) c3(3-phosphoinositide-dependent protein kinase 1 isoform 1) c4(130227, 130228) c5(130229, 130230, 130231, 130232, 130233, 130234, 130235, 130236, 130237, 130238, 130239, 130240) c6( ) c7(T, ht, NT, aA, bB, fd, bS, v, ci, Fb, fr, cd, q, ad, bv, hd, bpj, aT, h, hu, ask, ae, aW, aml, Y, r, Qq, kp, aF, g, bz, ck, Qi, nM, eM, nB, bE, mx, bt, B); #c1(PDPK1) c2(NP_112558) c3(3-phosphoinositide-dependent protein kinase 1 isoform 2) c4(221389) c5(221390, 221391, 221392, 221393, 221394, 221395) c6( ) c7(T, ht, NT, aA, bB, fd, bS, v, ci, Fb, fr, cd, q, ad, bv, hd, bpj, aT, h, hu, ask, ae, aW, aml, Y, r, Qq, kp, aF, g, bz, ck, Qi, nM, eM, nB, bE, mx, bt, B); #c1(PDPR) c2(NP_060460) c3(pyruvate dehydrogenase phosphatase regulatory subunit, mitochondrial precursor) c4(196050, 196051) c5(196052, 196053, 196054, 196055, 196056, 196057, 196058, 196059, 196060, 196061, 196062, 196063, 196064) c6( ) c7(abV); #c1(PDPR) c2(XP_005256076) c3(pyruvate dehydrogenase phosphatase regulatory subunit, mitochondrial isoform X6) c4( ) c5(273937, 273938, 273939, 273940, 273941, 273942) c6( ) c7(abV); #c1(PDPR) c2(XP_006721287) c3(pyruvate dehydrogenase phosphatase regulatory subunit, mitochondrial isoform X2) c4(293910) c5(293911, 293912, 293913, 293914, 293915, 293916) c6( ) c7(abV); #c1(PDPR) c2(XP_006721288) c3(pyruvate dehydrogenase phosphatase regulatory subunit, mitochondrial isoform X3) c4(293917) c5( ) c6( ) c7(abV); #c1(PDPR) c2(XP_011521492) c3(pyruvate dehydrogenase phosphatase regulatory subunit, mitochondrial isoform X4) c4(316240) c5(316241, 316242, 316243, 316244, 316245) c6( ) c7(abV); #c1(PDRG1) c2(NP_110442) c3(p53 and DNA damage-regulated protein 1) c4(220383) c5(220384, 220385, 220386, 220387, 220388, 220389) c6( ) c7(bB, bE, T, aq, K); #c1(PDS5A) c2(NP_001093869) c3(sister chromatid cohesion protein PDS5 homolog A isoform 1) c4(40461) c5(40462, 40463, 40464, 40465, 40466, 40467) c6( ) c7(aNL, T, cd, ac, ey); #c1(PDS5A) c2(NP_001093870) c3(sister chromatid cohesion protein PDS5 homolog A isoform 3) c4(40468) c5(40469, 40470, 40471, 40472, 40473, 40474) c6( ) c7(aNL, T, cd, ac, ey); #c1(PDS5B) c2(NP_055847) c3(sister chromatid cohesion protein PDS5 homolog B) c4(183893) c5(183894, 183895, 183896, 183897, 183898, 183899, 183900, 183901, 183902, 183903, 183904, 183905) c6( ) c7(T, aW, r, aT, aNL, it, K, cm, ae, af, ad); #c1(PDS5B) c2(XP_005266355) c3(sister chromatid cohesion protein PDSS homolog B isoform X3) c4(279965) c5( ) c6( ) c7(T, aW, r, aT, aNL, it, K, cm, ae, af, ad); #c1(PDSS1) c2(NP_055132) c3(decaprenyl-diphosphate synthase subunit 1) c4(179173, 179174, 179175) c5(179176, 179177, 179178, 179179, 179180, 179181, 179182, 179183, 179184, 179185, 179186, 179187, 179188, 179189, 179190, 179191, 179192, 179193, 179194) c6( ) c7(EG, b, iW, aM, h, EF, gl, i, cpA); #c1(PDSS2) c2(NP_065 (14) c3 (decaprenyl-diphosphate synthase subunit 2) c4(203984) c5(203985, 203986, 203987, 203988, 203989, 203990, 203991) c6( ) c7(EG, sk, eR, eM, cpB, Y, vw, EF, dy, ah, bz, NN, ajU); #c1(PDSS2) c2(XP_011534263) c3(decaprenyl-diphosphate synthase subunit 2 isoform X6) c4(330620) c5(330621, 330622, 330623, 330624, 330625, 330626) c6( ) c7(EG, sk, eR, eM, cpB, Y, vw, EF, dy, ah, bz, NN, ajU); #c1(PDSS2) c2(XP_011534264) c3(decaprenyl-diphosphate synthase subunit 2 isoform X7) c4(330627, 330628) c5(330629, 330630, 330631, 330632, 330633, 330634, 330635, 330636, 330637, 330638, 330639) c6( ) c7(EG, sk, eR, eM, cpB, Y, vw, EF, dy, ah, bz, NN, ajU); #c1(PDSS2) c2(XP_011534265) c3(decaprenyl-diphosphate synthase subunit 2 isoform X8) c4(330640) c5(330641, 330642, 330643, 330644, 330645, 330646) c6( ) c7(EG, sk, eR, eM, cpB, Y, vw, EF, dy, ah, bz, NN, ajU); #c1(PDX1) c2(NP_000200) c3(pancreas/duodenum homeobox protein 1) c4(4457) c5(4458, 4459, 4460, 4461, 4462, 4463, 4464, 4465) c6( ) c7(bL, T, aC, eR, pY, aD, aw, aA, aj, dG, v, ci, aNH, eJ, ad, J, mj, jz, mh, aE, pX, MP, cpD, cpC, jy, K, bxf, bq, aKG, pl, eG, A, cH, cpw, EU, kp, aF, LV, cM, mi, Vy, mg, mf, ei); #c1(PDXDC1) c2(NP_001272374) c3(pyridoxal-dependent decarboxylase domain-containing protein 1 isoform 3) c4(104013) c5(104014, 104015, 104016, 104017, 104018, 104019) c6( ) c7(fr, ey); #c1(PDXDC1) c2(NP_001272376) c3(pyridoxal-dependent decarboxylase domain-containing protein 1 isoform 4) c4( ) c5(104020, 104021, 104022, 104023, 104024) c6( ) c7(fr, ey); #c1(PDXDC1) c2(NP_001272377) c3(pyridoxal-dependent decarboxylase domain-containing protein 1 isoform 5) c4(104025) c5(104026, 104027, 104028, 104029, 104030, 104031) c6( ) c7(fr, ey); #c1(PDXDC1) c2(NP_001272378) c3(pyridoxal-dependent decarboxylase domain-containing protein 1 isoform 6) c4(104032, 104033) c5(104034, 104035, 104036, 104037, 104038, 104039, 104040, 104041, 104042, 104043, 104044, 104045, 104046) c6( ) c7(fr, ey); #c1(PDXDC1) c2(NP_001272379) c3 (pyridoxal-dependent decarboxylase domain-containing protein 1 isoform 7) c4(104047) c5(104048, 104049, 104050, 104051, 104052, 104053) c6( ) c7(fr, ey); #c1(PDXDC1) c2(NP_055842) c3(pyridoxal-dependent decarboxylase domain-containing protein 1 isoform 1) c4(183848, 183849) c5(183850, 183851, 183852, 183853, 183854, 183855) c6( ) c7(fr, ey); #c1(PDXK) c2(NP_003672) c3(pyridoxal kinase) c4(140088, 140089) c5(140090, 140091, 140092, 140093, 140094, 140095, 140096, 140097, 140098, 140099, 140100, 140101) c6( ) c7(T, eM, fQ, an, f, cB); #c1(PDXP) c2(NP_064711) c3(pyridoxal phosphate phosphatase) c4(203528, 203529) c5(203530, 203531, 203532, 203533, 203534, 203535, 203536, 203537, 203538, 203539, 203540, 203541) c6( ) c7(T, eR, aEj, QO, dM, if, cz, sM, n, bB, GC, ci, qN, mF, aFJ, q, aR, ke, fm, akU, yg, aT, K, YQ, cb, akV, aW, ff, eH, r, en, g, eF, bz, dr, asE, cpE, KE, aJH, TJ, wE, Da, m, bee, cn); #c1(PDYN) c2(XP_011527552) c3(proenkephalin-B isoform X1) c4(322361) c5(322362, 322363, 322364, 322365, 322366, 322367, 322368, 322369, 322370, 322371, 322372, 322373, 322374, 322375) c6( ) c7(aoU, T, dD, lB, jd, ha, bO, cz, lb, To, rf, bS, j, ct, Kh, gf, Ge, aqQ, eq, il, VV, aR, FY, aqt, gM, cF, ot, KB, cpF, cpH, Gb, h, yZ, bK, K, an, he, aTO, xa, alL, af, hZ, Ng, alM, cpG, ff, rU, gC, cpl, cu, aFx, en, Z, hJ, cM, xw, eE, cm, FT, cv, ok, ky, kB, cA, Kv, aYb, dJ, Gs, U, abl, aqm, nw, aax, L); #c1(PDZD2) c2(NP_835260) c3(PDZ domain-containing protein 2) c4(253443, 253444) c5(253445, 253446, 253447, 253448, 253449, 253450, 253451, 253452, 253453, 253454, 253455, 253456, 253457, 253458, 253459, 253460) c6( ) c7(gh, T, bM, AS, Ol, fG, if, cpJ, jh, n, fr, ci, fu, qN, cd, ad, eA, hX, Hx, py, MR, WY, sO, aT, h, K, jj, an, ae, af, bc, qw, d, aW, oV, vj, fc, Du, ff, akB, hN, aaE, en, cm, ik, bz, ck, es, ar, c, nB, eM, iA, U, mf, jM, cP, fD, y, bt, it, B); #c1(PDZD2) c2(XP_006714523) c3(PDZ domain-containing protein 2 isoform X7) c4( ) c5(287892) c6( ) c7(gh, T, bM, AS, Ol, fG, if, cpJ, jh, n, fr, ci, fu, qN, cd, ad, eA, hX, Hx, py, MR, WY, sO, aT, h, K, jj, an, ae, af, bc, qw, d, aW, oV, vj, fc, Du, ff, akB, hN, aaE, en, cm, ik, bz, ck, es, ar, c, nB, eM, iA, U, mf, jM, cP, fD, y, bt, it, B); #c1(PDZD2) c2(XP_011512295) c3(PDZ domain-containing protein 2 isoform X2) c4( ) c5(302829, 302830, 302831, 302832) c6( ) c7(gh, T, bM, AS, Ol, fG, if, cpJ, jh, n, fr, ci, fu, qN, cd, ad, eA, hX, Hx, py, MR, WY, sO, aT, h, K, j, an, ae, af, bc, qw, d, aW, oV, vj, fc, Du, ff, akB, hN, aaE, en, cm, iK, bz, ck, es, ar, c, nB, eM, iA, U, mf, jM, cP, fD, y, bt, it, B); #c1(PDZD2) c2(XP_011512296) c3(PDZ domain-containing protein 2 isoform X3) c4(302833) c5( ) c6( ) c7(gh, T, bM, AS, Ol, fG, if, cpJ, jh, n, fr, ci, fu, qN, cd, ad, eA, hX, Hx, py, MR, WY, sO, aT, h, K, jj, an, ae, af, bc, qw, d, aW, oV, vj, fc, Du, ff, akB, hN, aaE, en, cm, iK, bz, ck, es, ar, c, nB, eM, iA, U, mf, jM, cP, fD, y, bt, it, B); #c1(PDZD4) c2(NP_001290441) c3(PDZ domain-containing protein 4 isoform 1) c4(115457) c5(115458, 115459, 115460, 115461, 115462, 115463) c6( ) c7(iJ, fN); #c1(PDZD4) c2(NP_001290442) c3(PDZ domain-containing protein 4 isoform 3) c4(115464) c5(115465, 115466, 115467, 115468, 115469, 115470) c6( ) c7(iJ, fN); #c1(PDZD4) c2(NP_001290443) c3(PDZ domain-containing protein 4 isoform 4) c4(115471) c5(115472, 115473, 115474, 115475, 115476, 115477, 115478) c6( ) c7(iJ, fN); #c1(PDZD4) c2(NP_115901) c3(PDZ domain-containing protein 4 isoform 2) c4(224578) c5(224579, 224580, 224581, 224582, 224583, 224584, 224585, 224586) c6( ) c7(iJ, fN); #c1(PDZD7) c2(NP_001182192) c3(PDZ domain-containing protein 7 isoform 1) c4(74679, 74680) c5(74681, 74682, 74683, 74684, 74685, 74686) c6( ) c7(lR, alA, FQ, oc, FM, cpK); #c1(PDZD7) c2(NP_079171) c3(PDZ domain-containing protein 7 isoform 2) c4(217291) c5(217292, 217293, 217294, 217295, 217296, 217297) c6( ) c7(lR, alA, FQ, oc, FM, cpk); #c1(PDZD7) c2(XP_005270222) c3(PDZ domain-containing protein 7 isoform X4) c4( ) c5(283289) c6( ) c7(lR, alA, FQ, oc, FM, cpk); #c1(PDZD7) c2(XP_011538481) c3(PDZ domain-containing protein 7 isoform X3) c4( ) c5(336236, 336237, 336238, 336239, 336240, 336241) c6( ) c7(lR, alA, FQ, oc, FM, cpk); #c1(PDZD8) c2(NP_776152) c3(PDZ domain-containing protein 8) c4(250863) c5(250864, 250865, 250866, 250867, 250868, 250869) c6( ) c7(h, gn); #c1(PDZK1) c2(XP_011507918) c3(Na(+)/H(+) exchange regulatory cofactor NHE-RF3 isoform X1) c4(298233) c5( ) c6( ) c7(nD, T, dD, nn, aW, ea, e, jx, qK, aT, fd, hN, aq, fe, j, ci, ei, eq, B, ad); #c1(PDZK1) c2(XP_011507921) c3 (Na(+)/H(+) exchange regulatory cofactor NHE-RF3 isoform X3) c4( ) c5(298234, 298235, 298236, 298237, 298238, 298239) c6( ) c7(nD, T, dD, nn, aW, ea, e, jx, qK, aT, fd, hN, aq, fe, j, ci, ei, eq, B, ad); #c1(PDZRN3) c2(NP_001290070) c3(E3 ubiquitin-protein ligase PDZRN3 isoform 4) c4(114968) c5(114969, 114970, 114971, 114972, 114973, 114974) c6( ) c7(bL, aw, m); #c1(PDZRN3) c2(NP_001290071) c3(E3 ubiquitin-protein ligase PDZRN3 isoform 5) c4(114975) c5(114976, 114977, 114978, 114979, 114980, 114981) c6( ) c7(bL, aw, m); #c1(PDZRN3) c2(NP_055824) c3(E3 ubiquitin-protein ligase PDZRN3 isoform 1) c4(183710, 183711) c5(183712, 183713, 183714, 183715, 183716, 183717, 183718, 183719, 183720, 183721, 183722, 183723) c6( ) c7(bL, aw, m); #c1(PDZRN4) c2(NP_001158067) c3(PDZ domain-containing RlNG finger protein 4 isoform 1) c4(63075) c5(63076, 63077, 63078, 63079, 63080, 63081, 63082) c6( ) c7(V, bK, ac, c, an); #c1(PDZRN4) c2(NP_037509) c3(PDZ domain-containing RlNG finger protein 4 isoform 2) c4(175926, 175927) c5(175928, 175929, 175930, 175931, 175932, 175933, 175934, 175935, 175936, 175937, 175938, 175939) c6( ) c7(V, bK, ac, c, an); #c1(PEA15) c2(NP_001284505) c3(astrocytic phosphoprotein PEA-15 isoform a) c4(111944) c5(111945, 111946, 111947, 111948, 111949, 111950, 111951) c6( ) c7(bL, T, aZ, qy, aj, fr, lu, q, J, eA, av, hd, lg, aT, af, kl, d, aW, as, t, cm, bz, eM, nB, dB, wO, hk, cj, m); #c1(PEA15) c2(NP_001284506) c3(astrocytic phosphoprotein PEA-15 isoform b) c4(111952) c5(111953, 111954, 111955, 111956, 111957) c6( ) c7(bL, T, aZ, qy, aj, fr, lu, q, J, eA, av, hd, lg, aT, af, kl, d, aW, as, t, cm, bz, eM, nB, dB, wO, hk, cj, m); #c1(PEA15) c2(NP_001284507) c3(astrocytic phosphoprotein PEA-15 isoform c) c4(111958) c5(111959, 111960, 111961, 111962, 111963, 111964) c6( ) c7(bL, T, aZ, qy, aj, fr, lu, q, J, eA, av, hd, lg, aT, af, kl, d, aW, as, t, cm, bz, eM, nB, dB, wO, hk, cj, m); #c1 (PEAK1) c2(NP_079052) c3(pseudopodium-enriched atypical kinase 1) c4(216759) c5(216760, 216761, 216762, 216763, 216764, 216765, 216766, 216767, 216768, 216769) c6( ) c7(LV, T, kp, aE, aT); #c1(PEAR1) c2(NP_001073940) c3(platelet endothelial aggregation receptor 1 precursor) c4(36617, 36618) c5(36619, 36620, 36621, 36622, 36623, 36624, 36625, 36626, 36627, 36628, 36629, 36630, 36631, 36632, 36633, 36634) c6( ) c7(kD, j); #c1(PEAR1) c2(XP_011507811) c3(platelet endothelial aggregation receptor 1 isoform X2) c4( ) c5(297953) c6( ) c7(kD, j); #c1(PEAR1) c2(XP_011507814) c3(platelet endothelial aggregation receptor 1 isoform X4) c4( ) c5(297954) c6( ) c7(kD, j); #c1(PEBP1) c2(NP_002558) c3(phosphatidylethanolamine-binding protein 1 preproprotein) c4(129798, 129799) c5(129800, 129801, 129802, 129803, 129804, 129805, 129806, 129807, 129808, 129809, 129810, 129811) c6( ) c7(T, jv, if, aw, ht, jh, aA, bB, n, fd, j, ci, qN, zP, q, ad, eA, aDF, eJ, biq, b, aE, i, ds, aT, K, hu, fb, ae, af, WW, aol, zQ, iU, aW, Y, ff, az, r, ks, aq, af, en, g, wA, t, axp, cm, kP, eM, bE, np, m, adg, bt, Pr, B); #c1(PEBP4) c2(NP_659399) c3(phosphatidylethanolamine-binding protein 4 precursor) c4(239756) c5(239757, 239758, 239759, 239760, 239761, 239762) c6( ) c7(T, fM, eM, aW, r, aT, cz, j, t, bz, q); #c1(PECAM1) c2(XP_005276938) c3(platelet endothelial cell adhesion molecule isoform X3) c4(285742) c5( ) c6( ) c7(lD, bM, T, ea, e, fd, ci, Qp, x, xE, aE, Bc, M, dV, Ds, K, eN, ly, DA, aW, aDn, zC, g, as, cm, uC, eM, nB, iA, vy, cj, k, m, eg, Vj, P, aov, dD, lT, aw, n, BH, fr, Cd, eh, cd, ai, eW, aXl, pe, kl, lG, By, Jn, DU, pl, cY, CM, sm, fe, kp, mo, op, lx, kT, dB, Vy, it, B, Dw, dA, eR, dK, lH, cR, j, AO, Cb, V, eA, nX, aT, cy, jj, bq, lM, ae, af, qk, ak, bf, s, iU, Y, bg, eS, t, Vq, bN, ck, bvL, beH, beJ, ac, dh, vi, sY, MH, ow, eq, atK, ad, afb, Jm, hd, CJ, py, qt, eZ, iV, bz, bF, es, Hw, bE, aS, ei, lf, ne, y); #c1(PECAM1) c2(XP_005276939) c3(platelet endothelial cell adhesion molecule isoform X4) c4( ) c5(285743) c6( ) c7(lD, bM, T, ea, e, fd, ci, Qp, x, xE, aE, Bc, M, dV, Ds, K, eN, ly, DA, aW, aDn, zC, g, as, cm, uC, eM, nB, iA, vy, cj, k, m, eg, Vj, P, aov, dD, lT, aw, n, BH, fr, Cd, eh, cd, ai, eW, aXl, pe, kl, lG, By, Jn, DU, pl, cY, CM, sm, fe, kp, mo, op, lx, kT, dB, Vy, it, B, Dw, dA, eR, dK, lH, cR, j, AO, Cb, V, eA, nX, aT, cy, j, bq, lM, ae, af, qk, ak, bf, s, iU, Y, bg, eS, t, Vq, bN, ck, bvL, beH, beJ, ac, dh, vi, sY, MH, ow, eq, atK, ad, afb, Jm, hd, CJ, py, qt, eZ, iV, bz, bF, es, Hw, bE, aS, ei, lf, ne, y); #c1(PECAM1) c2(XP_005276940) c3(platelet endothelial cell adhesion molecule isoform X5) c4( ) c5(285744, 285745, 285746) c6( ) c7(lD, bM, T, ea, e, fd, ci, Qp, x, xE, aE, Bc, M, dV, Ds, K, eN, ly, DA, aW, aDn, zC, g, as, cm, uC, eM, nB, iA, vy, cj, k, m, eg, Vj, P, aov, dD, lT, aw, n, BH, fr, Cd, eh, cd, ai, eW, aXl, pe, kl, lG, By, Jn, DU, pl, cY, CM, sm, fe, kp, mo, op, lx, kT, dB, Vy, it, B, Dw, dA, eR, dK, lH, cR, j, AO, Cb, V, eA, nX, aT, cy, jj, bq, lM, ae, af, qk, ak, bf, s, iU, Y, bg, eS, t, Vq, bN, ck, bvL, beH, beJ, ac, dh, vi, sY, MH, ow, eq, atK, ad, afb, Jm, hd, CJ, py, qt, eZ, iV, bz, bF, es, Hw, bE, aS, ei, lf, ne, y); #c1(PECAM1) c2(XP_011523192) c3(platelet endothelial cell adhesion molecule isoform X1) c4(317717, 317718, 317719) c5(317720, 317721, 317722, 317723, 317724, 317725) c6( ) c7(lD, bM, T, ea, e, fd, ci, Qp, x, xE, aE, Bc, M, dV, Ds, K, eN, ly, DA, aW, aDn, zC, g, as, cm, uC, eM, nB, iA, vy, cj, k, m, eg, Vj, P, aov, dD, lT, aw, n, BH, fr, Cd, eh, cd, ai, eW, aXl, pe, kl, lG, By, Jn, DU, pl, cY, CM, sm, fe, kp, mo, op, lx, kT, dB, Vy, it, B, Dw, dA, eR, dK, lH, cR, j, AO, Cb, V, eA, nX, aT, cy, jj, bq, lM, ae, af, qk, ak, bf, s, iU, Y, bg, eS, t, Vq, bN, ck, bvL, beH, beJ, ac, dh, vi, sY, MH, ow, eq, atK, ad, afb, Jm, hd, CJ, py, qt, eZ, iV, bz, bF, es, Hw, bE, aS, ei, lf, ne, y); #c1(PECR) c2(NP_060911) c3(peroxisomal trans-2-enoyl-CoA reductase) c4(198587) c5(198588, 198589, 198590, 198591, 198592, 198593) c6( ) c7(at, nA); #c1(PEG10) c2(NP_001035242) c3(retrotransposon-derived protein PEG10 isoform 2) c4(32657) c5(32658, 32659, 32660, 32661, 32662, 32663, 32664, 32665) c6( ) c7(T, aE, Qu, aW, Y, hl, cj, aT, aq, ci, iR, AO, B, ad); #c1(PEG10) c2(NP_001165908) c3(retrotransposon-derived protein PEG10 isoform 3) c4( ) c5(67995, 67996) c6( ) c7(T, aE, Qu, aW, Y, hl, cj, aT, aq, ci, iR, AO, B, ad); #c1(PEG3) c2(NP_001139657) c3(paternally-expressed gene 3 protein isoform 2) c4(59589) c5( ) c6( ) c7(T, avp, fM, aE, lg, CN, jz, aW, Y, if, iT, aT, bcd, cm, GA, m, qN, t, nJ, eA, ad); #c1(PEG3) c2(NP_001139658) c3(paternally-expressed gene 3 protein isoform 1) c4(59590, 59591) c5(59592, 59593, 59594, 59595, 59596, 59597, 59598, 59599, 59600, 59601, 59602, 59603, 59604, 59605, 59606, 59607, 59608, 59609, 59610, 59611, 59612, 59613, 59614, 59615, 59616, 59617, 59618, 59619, 59620, 59621, 59622) c6( ) c7(T, avp, fM, aE, lg, CN, jz, aW, Y, if, iT, aT, bed, cm, GA, m, qN, t, nJ, eA, ad); #c1(PEG3) c2(NP_001139659) c3(paternally-expressed gene 3 protein isoform 3) c4(59623) c5(59624, 59625, 59626, 59627, 59628) c6( ) c7(T, avp, fM, aE, lg, CN, jz, aW, Y, if, iT, aT, bcd, cm, GA, m, qN, t, nJ, eA, ad); #c1(PELI1) c2(XP_011531296) c3(E3 ubiquitin-protein ligase pellino homolog 1 isoform X1) c4(327293) c5(327294, 327295, 327296, 327297, 327298, 327299) c6( ) c7(bL, lf, gZ, aj, vc, lQ, Pk, J, mu); #c1(PELI2) c2(NP_067078) c3(E3 ubiquitin-protein ligase pellino homolog 2) c4(208938) c5(208939, 208940, 208941, 208942, 208943, 208944) c6( ) c7(ac); #c1(PELP1) c2(NP_001265170) c3(proline-, glutamic acid- and leucine-rich protein 1 isoform 2) c4(97912, 97913) c5(97914, 97915, 97916, 97917, 97918, 97919) c6( ) c7(ae, gj, T, aE, AO, ws, qt, aW, ajL, hL, aT, r, ci, m, g, t, ajB, bz, q, ad); #c1(PELP1) c2(NP_055204) c3(proline-, glutamic acid- and leucine-rich protein 1 isoform 1) c4(179782, 179783) c5(179784, 179785, 179786, 179787, 179788, 179789, 179790, 179791, 179792, 179793, 179794, 179795, 179796, 179797, 179798, 179799, 179800, 179801, 179802, 179803) c6( ) c7(ae, gj, T, aE, AO, ws, qt, aW, ajL, hL, aT, r, ci, m, g, t, ajB, bz, q, ad); #c1(PEMT) c2(NP_001254480) c3(phosphatidylethanolamine N-methyltransferase isoform 4) c4(94421) c5(94422, 94423) c6( ) c7(ng, T, jp, arQ, cz, aj, v, j, V, J, Na, aT, Nc, dL, bq, eL, vl, af, aW, fc, qa, cP, ei, B); #c1(PEMT) c2(NP_001254481) c3(phosphatidylethanolamine N-methyltransferase isoform 3) c4(94424, 94425) c5(94426, 94427, 94428, 94429, 94430, 94431, 94432, 94433, 94434, 94435, 94436, 94437, 94438, 94439, 94440, 94441, 94442, 94443) c6( ) c7(ng, T, jp, arQ, cz, aj, v, j, V, J, Na, aT, Nc, dL, bq, eL, vl, af, aW, fc, qa, cP, ei, B); #c1(PEMT) c2(NP_009100) c3(phosphatidylethanolamine N-methyltransferase isoform 2) c4( ) c5(170669, 170670, 170671, 170672, 170673, 170674, 170675, 170676) c6( ) c7(ng, T, jp, arQ, cz, aj, v, j, V, J, Na, aT, Nc, dL, bq, eL, vL, af, aW, fc, qa, cP, ei, B); #c1(PEMT) c2(NP_680477) c3(phosphatidylethanolamine N-methyltransferase isoform 1) c4(242298, 242299) c5(242300, 242301, 242302, 242303, 242304, 242305) c6( ) c7(ng, T, jp, arQ, cz, aj, v, j, V, J, Na, aT, Nc, dL, bq, eL, vl, af, aW, fc, qa, cP, ei, B); #c1(PENK) c2(NP_001129162) c3 (proenkephalin-A preproprotein) c4(50945, 50946, 50947, 50948, 50949) c5(50950, 50951, 50952, 50953, 50954, 50955, 50956, 50957, 50958, 50959, 50960, 50961, 50962, 50963, 50964, 50965, 50966, 50967, 50968, 50969, 50970, 50971) c6( ) c7(T, VR, hU, n, PJ, eJ, ad, aR, eW, cF, aE, Jb, SH, dL, bK, an, ae, aTO, eL, hZ, cpL, eH, kp, g, eF, LV, cm, wA, jb, FT, cv, pH, lB, aen); #c1(PEPD) c2(NP_000276) c3(xaa-Pro dipeptidase isoform 1) c4(6086) c5(6087, 6088, 6089, 6090, 6091, 6092) c6( ) c7(bL, T, AY, ac, fR, wL, Y, eZ, aT, CH, j, eo, wZ, fw); #c1(PEPD) c2(NP_001159528) c3(xaa-Pro dipeptidase isoform 2) c4(64244) c5(64245, 64246, 64247, 64248, 64249, 64250) c6( ) c7(bL, T, AY, ac, fR, wL, Y, eZ, aT, CH, j, eo, wZ, fw); #c1(PEPD) c2(NP_001159529) c3(xaa-Pro dipeptidase isoform 3) c4(64251) c5(64252, 64253, 64254, 64255, 64256, 64257) c6( ) c7(bL, T, AY, ac, fR, wL, Y, eZ, aT, CH, j, eo, wZ, fw); #c1(PER1) c2(NP_002607) c3(period circadian protein homolog 1) c4(130248, 130249) c5(130250, 130251, 130252, 130253, 130254, 130255, 130256, 130257, 130258, 130259, 130260, 130261, 130262) c6( ) c7(T, aC, e, al, Jd, qj, ci, Na, XW, AW, aE, Gb, K, eL, aiV, aW, hl, bxx, bMC, eM, dc, wE, pJ, aw, bzb, aj, ct, nA, h, aNU, dL, he, Gc, Jj, agG, CQ, asE, hR, uz, bD, B, bL, eR, cz, arD, v, j, q, J, eA, aaF, nv, aT, bK, ae, iU, r, cl, f, ik, Ym, bN, ck, iM, dy, gT, jN, vm, bS, dG, aZE, ad, fU, sO, fb, Xb, je, nf, yy, Z, bz, apM, bE, ZD, ei, y, pC); #c1(PER2) c2(NP_073728) c3(period circadian protein homolog 2) c4(213257, 213258, 213259, 213260) c5(213261, 213262, 213263, 213264, 213265, 213266, 213267, 213268, 213269, 213270, 213271, 213272, 213273, 213274, 213275, 213276, 213277, 213278, 213279, 213280, 213281, 213282, 213283, 213284, 213285, 213286, 213287, 213288, 213289, 213290) c6( ) c7(bL, T, aXN, dA, jx, jh, cpO, Nt, cR, Gd, cpM, bS, cQ, ct, j, v, bB, ww, VV, q, ad, eA, aqN, hb, aT, h, cy, aXO, bK, K, he, ae, af, Jj, aqv, tb, iU, LC, aW, r, aPZ, agG, fe, kp, cl, Z, Gi, Ym, cpP, FT, aPY, cpN, qJ, NC, ac, nM, iT, bE, cj, ei, pk, y, bt, B); #c1(PER3) c2(NP_001276790) c3(period circadian protein homolog 3 isoform 2) c4(108373) c5( ) c6( ) c7(oO, T, Nt, bB, aj, v, q, J, ad, x, Na, NS, aqk, aT, ta, h, bK, K, ae, d, B, iU, aW, aaB, r, aPZ, fe, NR, sz, Z, iV, ik, aQb, NC, cA, bE, Fi, bzm, k, jd, X); #c1(PER3) c2(NP_001276791) c3(period circadian protein homolog 3 isoform 1) c4(108374) c5(108375, 108376, 108377, 108378, 108379, 108380) c6( ) c7(oO, T, Nt, bB, aj, v, q, J, ad, x, Na, NS, aqk, aT, ta, h, bK, K, ae, d, B, iU, aW, aaB, r, aPZ, fe, NR, sz, Z, iV, ik, aQb, NC, cA, bE, Fi, bzm, k, jd, X); #c1(PER3) c2(NP_001276792) c3 (period circadian protein homolog 3 isoform 3) c4( ) c5(108381, 108382, 108383, 108384, 108385, 108386) c6( ) c7(oO, T, Nt, bB, aj, v, q, J, ad, x, Na, NS, aqk, aT, ta, h, bK, K, ae, d, B, iU, aW, aaB, r, aPZ, fe, NR, sz, Z, iV, ik, aQb, NC, cA, bE, Fi, bzm, k, jd, X); #c1(PERM1) c2(NP_001278296) c3(PGC-1 and ERR-induced regulator in muscle protein 1 isoform 2) c4(109545) c5(109546, 109547, 109548, 109549, 109550, 109551, 109552, 109553) c6( ) c7(aNG, bl); #c1(PERP) c2(NP_071404) c3(p53 apoptosis effector related to PMP-22) c4(211452, 211453, 211454) c5(211455, 211456, 211457, 211458, 211459, 211460, 211461, 211462, 211463, 211464, 211465, 211466, 211467, 211468, 211469, 211470, 211471, 211472) c6( ) c7(T, zQ, Y, ff, aT, auN, en, t, bz, iu, iP); #c1(PES1) c2(NP_001230154) c3(pescadillo homolog isoform 2) c4(85993) c5(85994, 85995, 85996, 85997, 85998, 85999) c6( ) c7(bE, aW, aT, bB, m, t); #c1(PES1) c2(NP_001269257) c3(pescadillo homolog isoform 3) c4(100924) c5(100925, 100926, 100927, 100928, 100929, 100930, 100931, 100932) c6( ) c7(bE, aW, aT, bB, m, t); #c1(PES1) c2(NP_055118) c3(pescadillo homolog isoform 1) c4(179064) c5(179065, 179066, 179067, 179068, 179069, 179070, 179071, 179072) c6( ) c7(bE, aW, aT, bB, m, t); #c1(PET100) c2(NP_001164626) c3(protein PET100 homolog, mitochondrial) c4(67074, 67075) c5(67076, 67077, 67078, 67079, 67080, 67081, 67082, 67083, 67084, 67085, 67086, 67087, 67088, 67089, 67090, 67091) c6( ) c7(acJ, ajU); #c1(PET1I7) c2(NP_001158283) c3(protein PET1I7 homolog, mitochondrial precursor) c4(63536) c5(63537, 63538, 63539, 63540, 63541, 63542, 63543) c6( ) c7(aSP); #c1(PEX1) c2(NP_000457) c3(peroxisome biogenesis factor 1 isoform 1) c4(9256) c5(9257, 9258, 9259, 9260, 9261, 9262, 9263, 9264) c6( ) c7(ajx, fc, mE, cpQ, aT, ml, Nh, aT, aR); #c1(PEX1) c2(NP_001269606) c3(peroxisome biogenesis factor 1 isoform 2) c4(102187, 102188) c5(102189, 102190, 102191, 102192, 102193, 102194, 102195, 102196, 102197, 102198, 102199) c6( ) c7(ajx, fc, mE, cpQ, aT, mL, Nh, aT, aR); #c1(PEX1) c2(NP_001269607) c3(peroxisome biogenesis factor 1 isoform 3) c4(102200) c5( ) c6( ) c7(ajx, fc, mE, cpQ, aT, ml, Nh, aT, aR); #c1(PEX1) c2(XP_005250490) c3 (peroxisome biogenesis factor 1 isoform X1) c4( ) c5(270355, 270356, 270357, 270358, 270359) c6( ) c7(ajx, fc, mE, cpQ, aT, ml, Nh, aT, aR); #c1(PEX11A) c2(NP_003838) c3(peroxisomal membrane protein 11A isoform 1) c4(141253) c5(141254, 141255, 141256, 141257, 141258, 141259) c6( ) c7(c); #c1(PEX12) c2(NP_000277) c3(peroxisome assembly protein (2) c4(6093, 6094) c5(6095, 6096, 6097, 6098, 6099, 6100, 6101, 6102, 6103, 6104, 6105, 6106) c6( ) c7(cpR, ajx, mE, mL, cpS, bX, al); #c1(PEX13) c2(NP_002609) c3(peroxisome biogenesis factor 13) c4(130263) c5(130264, 130265, 130266, 130267, 130268, 130269) c6( ) c7(cpU, cpT, cpQ, aT, mL, dF, al); #c1(PEX14) c2(XP_011539880) c3(peroxisomal membrane protein PEX14 isoform X2) c4( ) c5(338154) c6( ) c7(cpV, aW, ajP, aT, ml, oQ, cm, al); #c1(PEX16) c2(NP_004804) c3(peroxisomal biogenesis factor 16 isoform 1) c4(149973) c5(149974, 149975, 149976, 149977, 149978, 149979) c6( ) c7(cpX, aT, ml, cpW); #c1(PEX16) c2(NP_476515) c3(peroxisomal biogenesis factor 16 isoform 2) c4(230715) c5(230716, 230717, 230718, 230719, 230720, 230721) c6( ) c7(cpX, aT, ml, cpW); #c1(PEX19) c2(NP_001180573) c3(peroxisomal biogenesis factor 19 isoform c) c4(74268, 74269) c5(74270, 74271, 74272, 74273, 74274, 74275, 74276, 74277, 74278, 74279, 74280) c6( ) c7(cpY, ib, eM, bO, ml, LR, al); #c1(PEX19) c2(NP_002848) c3(peroxisomal biogenesis factor 19 isoform a) c4(132763, 132764, 132765) c5(132766, 132767, 132768, 132769, 132770, 132771, 132772, 132773, 132774, 132775, 132776, 132777, 132778, 132779, 132780, 132781, 132782, 132783, 132784) c6( ) c7(cpY, ib, eM, bO, mL, LR, al); #c1(PEX2) c2(NP_001165558) c3(peroxisome biogenesis factor 2) c4(67775, 67776) c5(67777, 67778, 67779, 67780, 67781, 67782, 67783, 67784, 67785, 67786, 67787, 67788, 67789, 67790, 67791, 67792, 67793, 67794, 67795, 67796, 67797, 67798, 67799, 67800, 67801) c6( ) c7(cpZ, ac, LC, cqa, ajx, mE, cy, ao, v, mL, cm, al, ad); #c1(PEX26) c2(NP_001121121) c3(peroxisome assembly protein 26 isoform a) c4(45249, 45250) c5(45251, 45252, 45253, 45254, 45255, 45256, 45257, 45258, 45259, 45260, 45261, 45262, 45263, 45264, 45265, 45266) c6( ) c7(ajx, cqc, cpQ, cqb, mL, dF, al); #c1(PEX26) c2(NP_001186248) c3(peroxisome assembly protein 26 isoform b) c4(77444) c5(77445, 77446, 77447, 77448, 77449, 77450) c6( ) c7(ajx, cqc, cpQ, cqb, mL, dF, al); #c1(PEX3) c2(NP_003621) c3(peroxisomal biogenesis factor 3) c4(139676, 139677) c5(139678, 139679, 139680, 139681, 139682, 139683, 139684, 139685, 139686, 139687, 139688, 139689, 139690, 139691, 139692, 139693) c6( ) c7(mE, al, ml, abh, cqd); #c1(PEX5) c2(NP_000310) c3(peroxisomal biogenesis factor 5 isoform b) c4(6921, 6922, 6923, 6924) c5(6925, 6926, 6927, 6928, 6929, 6930, 6931, 6932, 6933, 6934) c6( ) c7(dH, ajx, dw, mE, cpQ, aL, ml, cqf, cqe, al); #c1(PEX5) c2(NP_001124496) c3(peroxisomal biogenesis factor 5 isoform c) c4( ) c5(49163, 49164, 49165, 49166, 49167, 49168, 49169, 49/70, 49171, 49/72, 49173, 49174, 49175, 49176, 49177) c6( ) c7(dH, ajx, dw, mE, cpQ, aT, mL, cqf, cqe, al); #c1(PEX5) c2(NP_001287718) c3(peroxisomal biogenesis factor 5 isoform e) c4(112629, 112630) c5(112631, 112632, 112633, 112634, 112635, 112636, 112637, 112638, (12639, 112640) c6( ) c7(dH, ajx, dw, mE, cpQ, aL, mL, cqf, cqe, al); #c1(PEX5) c2(XP_005253512) c3(peroxisomal biogenesis factor 5 isoform X5) c4(272534) c5(272535, 272536, 272537, 272538, 272539, 272540, 272541, 272542, 272543) c6( ) c7(dH, ajx, dw, mE, cpQ, aL, mL, cqf, cqe, al); #c1(PEX5) c2(XP_011519096) c3(peroxisomal biogenesis factor 5 isoform X3) c4(313688) c5(313689) c6( ) c7(dH, ajx, dw, mE, cpQ, aL, mL, cqf, cqe, al); #c1(PEX5) c2(XP_011519098) c3(peroxisomal biogenesis factor 5 isoform X1) c4( ) c5(313690, 313691, 313692, 313693, 313694) c6( ) c7(dH, ajx, dw, mE, cpQ, aL, mL, cqf, cqe, al); #c1(PEX5) c2(XP_011519102) c3(peroxisomal biogenesis factor 5 isoform X2) c4(313695) c5(313696, 313697, 313698, 313699, 313700, 313701, 313702, 313703) c6( ) c7(dH, ajx, dw, mE, cpQ, aL, mL, cqf, cqe, al); #c1(PEX5) c2(XP_011519103) c3(peroxisomal biogenesis factor 5 isoform X4) c4(313704) c5(313705, 313706, 313707, 313708, 313709, 313710) c6( ) c7(dH, ajx, dw, mE, cpQ, aL, mL, cqf, cqe, al); #c1(PEX5L) c2(NP_001243679) c3(PEX5-related protein isoform 2) c4(91355, 91356) c5(91357, 91358, 91359, 91360, 91361, 91362, 91363, 91364, 91365, 91366, 91367, 91368, 91369, 91370, 91371, 91372, 91373) c6( ) c7(bL); #c1(PEX5L) c2(NP_001243680) c3(PEX5-related protein isoform 3) c4(91374) c5(91375, 91376, 91377, 91378, 91379, 91380) c6( ) c7(bL); #c1(PEX5L) c2(NP_001243681) c3(PEX5-related protein isoform 4) c4(91381) c5(91382, 91383, 91384, 91385, 91386, 91387) c6( ) c7(bL); #c1(PEX5L) c2(NP_001243684) c3(PEX5-related protein isoform 7) c4(91388) c5(91389, 91390, 91391, 91392, 91393, 91394) c6( ) c7(bL); #c1(PEX5L) c2(NP_057643) c3(PEX5-related protein isoform 1) c4(192011) c5(192012, 192013, 192014, 192015, 192016, 192017, 192018) c6( ) c7(bL); #c1(PEX5L) c2(XP_005247583) c3(PEX5-related protein isoform X10) c4(267867) c5(267868, 267869, 267870, 267871, 267872, 267873) c6( ) c7(bL); #c1(PEX5L) c2(XP_011511184) c3(PEX5-related protein isoform X3) c4(301658) c5( ) c6( ) c7(bL); #c1(PEX6) c2(NP_000278) c3(peroxisome biogenesis factor 6) c4(6107, 6108) c5(6109, 6110, 6111, 6112, 6113, 6114, 6115, 6116, 6117, 6118, 6119, 6120) c6( ) c7(cqh, cqg, ajx, mE, aL, mL, nA, oc, al, aR); #c1(PEX7) c2(NP_000279) c3(peroxisomal biogenesis factor 7) c4(6121, 6122) c5(6123, 6124, 6125, 6126, 6127, 6128, 6129, 6130, 6131, 6132, 6133, 6134) c6( ) c7(rR, ob, ajx, ahj, az, yB, cqi, aBn, LR, al); #c1(PEX7) c2(XP_011534202) c3(peroxisomal biogenesis factor 7 isoform X3) c4(330506) c5(330507, 330508, 330509, 330510, 330511, 330512) c6( ) c7(rR, ob, ajx, ahj, az, yB, cqi, aBn, LR, al); #c1(PF4) c2(NP_002610) c3(platelet factor 4 precursor) c4(130270, 130271, 130272) c5(130273, 130274, 130275, 130276, 130277, 130278) c6( ) c7(bM, T, dD, xu, dA, Dw, Yu, qb, e, jx, ez, cR, fd, j, eq, eh, ai, qh, aT, Ds, iY, DU, kU, cY, iU, aW, lh, mQ, hl, t, ck, bqd, gZ, m, fD, bt); #c1(PF4) c2(XP_005265753) c3(platelet factor 4 isoform X1) c4(279705) c5( ) c6( ) c7(bM, T, dD, xu, dA, Dw, Yu, qb, e, jx, ez, cR, fd, j, eq, eh, ai, qh, aT, Ds, iY, DU, kU, cY, iU, aW, lh, mQ, hl, t, ck, bqd, gZ, m, fD, bt); #c1(PF4V1) c2(NP_002611) c3 (platelet factor 4 variant precursor) c4(130279) c5(130280, 130281, 130282, 130283, 130284, 130285) c6( ) c7(gZ, aS, T, j, ao); #c1(PFDN4) c2(NP_002614) c3(prefoldin subunit 4) c4(130305) c5(130306, 130307, 130308, 130309, 130310, 130311) c6( ) c7(bP, aW, oe, EQ, r, aT, q); #c1(PFDN5) c2(NP_002615) c3(prefoldin subunit 5 isoform alpha) c4(130312) c5(130313, 130314, 130315, 130316, 130317, 130318, 130319, 130320, 130321, 130322, 130323) c6( ) c7(ck, nz, aE); #c1(PFDN5) c2(NP_665904) c3(prefoldin subunit 5 isoform gamma) c4(241963) c5(241964, 241965, 241966, 241967, 241968, 241969) c6( ) c7(ck, nz, aE); #c1(PFDN6) c2(NP_001252525) c3(prefoldin subunit 6) c4(94085, 94086) c5(94087, 94088, 94089, 94090, 94091, 94092, 94093, 94094, 94095, 94096, 94097, 94098, 94099, 94100, 94101, 94102, 94103, 94104, 94105, 94106, 94107, 94108, 94109, 94110, 94111, 94112, 94113, 94114, 94115, 94116, 94117, 94118, 94119, 94120, 94121, 94122, 94123, 94124, 94125, 94126, 94127, 94128, 94129, 94130, 94131, 94132, 94133, 94134, 94135, 94136, 94137, 94138, 94139, 94140, 94141, 94142, 94143, 94144, 94145, 94146, 94147, 94148, 94149, 94150, 94151, 94152, 94153, 94154, 94155, 94156, 94157, 94158, 94159, 94160, 94161, 94162, 94163, 94164, 94165, 94166, 94167, 94168, 94169, 94170, 94171, 94172, 94173) c6( ) c7(eZ); #c1(PFKFB1) c2(NP_001258733) c3(6-phosphofructo-2-kinase/fructose-2, 6-bisphosphatase 1 isoform 2) c4(96443) c5( ) c6( ) c7(qO); #c1(PFKFB1) c2(NP_001258734) c3(6-phosphofructo-2-kinase/fructose-2, 6-bisphosphatase 1 isoform 3) c4(96444) c5(96445, 96446, 96447, 96448, 96449, 96450, 96451, 96452, 96453, 96454, 96455, 96456) c6( ) c7(qO); #c1(PFKFB1) c2(NP_002616) c3(6-phosphofructo-2-kinase/fructose-2, 6-bisphosphatase 1 isoform 1) c4(130324) c5(130325, 130326, 130327, 130328, 130329, 130330) c6( ) c7(qO); #c1(PFKFB2) c2(NP_001018063) c3(6-phosphofructo-2-kinase/fructose-2, 6-bisphosphatase 2 isoform b) c4(27010, 27011, 27012) c5(27013, 27014, 27015, 27016, 27017, 27018, 27019, 27020, 27021, 27022, 27023, 27024, 27025, 27026, 27027, 27028, 27029, 27030) c6( ) c7(T, W); #c1(PFKFB2) c2(XP_011507930) c3(6-phosphofructo-2-kinase/fructose-2, 6-bisphosphatase 2 isoform X1) c4(298247, 298248) c5(298249, 298250, 298251, 298252, 298253, 298254, 298255, 298256, 298257, 298258, 298259, 298260) c6( ) c7(T, W); #c1(PFKFB3) c2(NP_001138915) c3(6-phosphofructo-2-kinase/fructose-2, 6-bisphosphatase 3 isoform 2) c4(58173, 58174) c5(58175, 58176, 58177, 58178, 58179, 58180, 58181, 58182, 58183, 58184, 58185, 58186) c6( ) c7(T, hd, d, ac, aC, bE, aW, tT, Y, ev, aT, cy, v, j, K, lQ, qM, as, Pk, y); #c1(PFKFB3) c2(NP_001269559) c3(6-phosphofructo-2-kinase/fructose-2, 6-bisphosphatase 3 isoform 3) c4(101971) c5(101972, 101973, 101974, 101975, 101976, 101977, 101978) c6( ) c7(T, hd, d, ac, aC, bE, aW, tT, Y, ev, aT, cy, v, j, K, lQ, qM, as, Pk, y); #c1(PFKFB3) c2(NP_004557) c3(6-phosphofructo-2-kinase/fructose-2, 6-bisphosphatase 3 isoform 1) c4(147622) c5(147623, 147624, 147625, 147626, 147627, 147628) c6( ) c7(T, hd, d, ac, aC, bE, aW, tT, Y, ev, aT, cy, v, j, K, lQ, qM, as, Pk, y); #c1(PFKFB3) c2(XP_005252521) c3(6-phosphofructo-2-kinase/fructose-2, 6-bisphosphatase 3 isoform X3) c4(271834) c5(271835) c6( ) c7(T, hd, d, ac, aC, bE, aW, tT, Y, ev, aT, cy, v, j, K, lQ, qM, as, Pk, y); #c1(PFKFB3) c2(XP_011517795) c3(6-phosphofructo-2-kinase/fructose-2, 6-bisphosphatase 3 isoform X4) c4(311675, 311676, 311677) c5(311678, 311679, 311680, 311681, 311682, 311683, 311684, 311685, 311686, 311687, 311688, 311689, 311690, 311691, 311692, 311693, 311694, 311695, 311696, 311697, 311698, 311699, 311700) c6( ) c7(T, hd, d, ac, aC, bE, aW, tT, Y, ev, aT, cy, v, j, K, lQ, qM, as, Pk, y); #c1(PFKFB4) c2(NP_004558) c3(6-phosphofructo-2-kinase/fructose-2, 6-bisphosphatase 4) c4(147629) c5(147630, 147631, 147632, 147633, 147634, 147635) c6( ) c7(T, aC, d, ae, eA, ad); #c1(PFKL) c2(NP_002617) c3(ATP-dependent 6-phosphofructokinase, liver type isoform b) c4(130331, 130332) c5(130333, 130334, 130335, 130336, 130337, 130338, 130339) c6( ) c7(Mg, mU, mW, acl, mZ, mS, yU, mY, bK, yZ, f, mV, mT, y, mX); #c1(PFKL) c2(XP_005261194) c3(ATP-dependent 6-phosphofructokinase, liver type isoform X5) c4( ) c5(277265, 277266, 277267, 277268, 277269) c6( ) c7(Mg, mU, mW, acl, mZ, mS, yU, mY, bK, yZ, f, mV, mT, y, mX); #c1(PFKL) c2(XP_006724075) c3(ATP-dependent 6-phosphofructokinase, liver type isoform X1) c4(295691, 295692) c5(295693, 295694, 295695, 295696, 295697, 295698, 295699, 295700, 295701, 295702, 295703, 295704) c6( ) c7(Mg, mU, mW, acl, mZ, mS, yU, mY, bK, yZ, f, mV, mT, y, mX); #c1(PFKM) c2(NP_001160158) c3(ATP-dependent 6-phosphofructokinase, muscle type isoform 1) c4(65119) c5(65120, 65121, 65122, 65123, 65124, 65125, 65126, 65127, 65128, 65129, 65130, 65131, 65132, 65133, 65134, 65135, 65136, 65137, 65138, 65139) c6( ) c7(bL, T, ajT, aW, oj, aT, j, ci, bq, add); #c1(PFKM) c2(NP_001160160) c3(ATP-dependent 6-phosphofructokinase, muscle type isoform 2) c4( ) c5(65140, 65141, 65142, 65143, 65144, 65145, 65146, 65147, 65148, 65149, 65150, 65151, 65152, 65153, 65154, 65155, 65156, 65157, 65158, 65159, 65160, 65161, 65162, 65163, 65164, 65165, 65166, 65167, 65168, 65169, 65/70) c6( ) c7(bL, T, ajT, aW, oj, aT, j, ci, bq, add); #c1(PFKM) c2(XP_005269033) c3(ATP-dependent 6-phosphofructokinase, muscle type isoform X4) c4(282258, 282259) c5(282260) c6( ) c7(bL, T, ajT, aW, oj, aT, j, ci, bq, add); #c1(PFKM) c2(XP_011536789) c3(ATP-dependent 6-phosphofructokinase, muscle type isoform X3) c4(334467, 334468, 334469, 334470, 334471, 334472) c5(334473, 334474, 334475, 334476, 334477, 334478, 334479, 334480, 334481, 334482, 334483, 334484, 334485, 334486) c6( ) c7(bL, T, ajT, aW, oj, aT, j, ci, bq, add); #c1(PFKP) c2(NP_001229268) c3(ATP-dependent 6-phosphofructokinase, platelet type isoform 2) c4(84099, 84100) c5(84101, 84102, 84103, 84104, 84105, 84106, 84107, 84108, 84109, 84110, 84111, 84112) c6( ) c7(aW, v, ajT, aT); #c1(PFKP) c2(NP_002618) c3(ATP-dependent 6-phosphofructokinase, platelet type isoform 1) c4(130340, 130341, 130342, 130343) c5(130344, 130345, 130346, 130347, 130348, 130349, 130350) c6( ) c7(aW, v, ajT, aT); #c1(PFKP) c2(XP_005252522) c3(ATP-dependent 6-phosphofructokinase, platelet type isoform X1) c4(271836, 271837) c5( ) c6( ) c7(aW, v, ajT, aT); #c1(PFKP) c2(XP_005252523) c3(ATP-dependent 6-phosphofructokinase, platelet type isoform X3) c4(271838) c5(271839, 271840, 271841, 271842, 271843, 271844) c6( ) c7(aW, v, ajT, aT); #c1(PFKP) c2(XP_005252524) c3(ATP-dependent 6-phosphofructokinase, platelet type isoform X4) c4( ) c5(271845, 271846, 271847, 271848, 271849, 271850, 271851, 271852, 271853, 271854, 271855, 271856, 271857, 271858, 271859, 271860, 271861, 271862, 271863, 271864, 271865, 271866, 271867, 271868, 271869, 271870, 271871, 271872, 271873) c6( ) c7(aW, v, ajT, aT); #c1(PFN1) c2(NP_005013) c3(profilin-1) c4(152165) c5(152166, 152167, 152168, 152169, 152170, 152171) c6( ) c7(T, Ol, Vd, ci, fr, PJ, ad, tP, aE, WY, aT, kf, By, K, ae, aW, aq, aaE, g, cqj, bb, kP, c, DW, U, rA, it, B); #c1(PFN2) c2(NP_002619) c3(profilin-2 isoform b) c4(130351) c5(130352, 130353, 130354, 130355, 130356, 130357) c6( ) c7(ds, aF, K, c, aA); #c1(PFN2) c2(NP_444252) c3(profilin-2 isoform a) c4(230111) c5(230112, 230113, 230114, 230115, 230116, 230117) c6( ) c7(ds, aF, K, c, aA); #c1(PGA3) c2(NP_001073275) c3(pepsin A-3 preproprotein) c4(35669, 35670, 35671) c5(35672, 35673, 35674, 35675, 35676, 35677, 35678, 35679, 35680, 35681, 35682, 35683, 35684, 35685, 35686, 35687, 35688, 35689, 35690, 35691, 35692) c6( ) c7(bvS, aF, aLA, aA); #c1(PGA4) c2(NP_001073276) c3(pepsin A-4 preproprotein) c4(35693, 35694) c5(35695, 35696, 35697, 35698, 35699, 35700, 35701, 35702, 35703, 35704, 35705, 35706, 35707) c6( ) c7(aLA); #c1(PGA5) c2(NP_055039) c3(pepsin A-5 preproprotein) c4(178447, 178448, 178449) c5(178450, 178451, 178452, 178453, 178454, 178455, 178456, 178457, 178458, 178459, 178460, 178461, 178462, 178463, 178464, 178465, 178466, 178467, 178468, 178469, 178470, 178471) c6( ) c7(fe, akY, aLA, iV); #c1(PGAM1) c2(NP_002620) c3(phosphoglycerate mutase 1) c4(130358, 130359) c5(130360, 130361, 130362, 130363, 130364, 130365, 130366) c6( ) c7(T, zQ, eM, iR, ff, oW, r, aA, aq, bK, ci, K, q, en, zP, g, eA, B); #c1(PGAM2) c2(NP_000281) c3(phosphoglycerate mutase 2) c4(6135) c5(6136, 6137, 6138, 6139, 6140, 6141) c6( ) c7(Bw, afU, cqk, K, akW, ad); #c1(PGAM4) c2(NP_001025062) c3(phosphoglycerate mutase 4) c4(29292) c5(29293, 29294, 29295, 29296, 29297, 29298) c6( ) c7(q, eA, r); #c1(PGAP1) c2(NP_079265) c3(GPI inositol-deacylase) c4(217627) c5(217628, 217629, 217630, 217631, 217632, 217633, 217634) c6( ) c7(bM, y, bN, an); #c1(PGAP1) c2(XP_011510181) c3(GPI inositol-deacylase isoform X2) c4(300747) c5(300748, 300749, 300750, 300751, 300752) c6( ) c7(bM, y, bN, an); #c1(PGAP1) c2(XP_011510182) c3(GPI inositol-deacylase isoform X3) c4(300753, 300754) c5(300755, 300756, 300757, 300758, 300759, 300760, 300761, 300762, 300763, 300764, 300765, 300766) c6( ) c7(bM, y, bN, an); #c1(PGAP2) c2(NP_001138910) c3(post-GPI attachment to proteins factor 2 isoform 2) c4(58138) c5(58139, 58140, 58141, 58142, 58143, 58144) c6( ) c7(T, com, eH, ml, cqn, K, nA, eF, cql, dr); #c1(PGAP2) c2(NP_001243165) c3(post-GPI attachment to proteins factor 2 isoform 4) c4(90024, 90025) c5(90026, 90027, 90028, 90029, 90030, 90031, 90032, 90033, 90034) c6( ) c7(T, cqm, eH, ml, cqn, K, nA, eF, cql, dr); #c1(PGAP2) c2(NP_001243166) c3(post-GPI attachment to proteins factor 2 isoform 5) c4(90035) c5(90036) c6( ) c7(T, cqm, eH, ml, cqn, K, nA, eF, cql, dr); #c1(PGAP2) c2(NP_001243167) c3(post-GPI attachment to proteins factor 2 isoform 6) c4(90037) c5(90038, 90039, 90040, 90041, 90042, 90043, 90044, 90045, 90046, 90047, 90048) c6( ) c7(T, cqm, eH, ml, cqn, K, nA, eF, cql, dr); #c1(PGAP2) c2(NP_001243169) c3(post-GPI attachment to proteins factor 2 isoform 8) c4(90049) c5(90050, 90051, 90052, 90053, 90054) c6( ) c7(T, com, eH, ml, cqn, K, nA, eF, cql, dr); #c1(PGAP2) c2(NP_001269967) c3(post-GPI attachment to proteins factor 2 isoform 9) c4(103302) c5(103303, 103304, 103305, 103306, 103307, 103308, 103309) c6( ) c7(T, com, eH, ml, cqn, K, nA, eF, cql, dr); #c1(PGAP2) c2(NP_055304) c3 (post-GPI attachment to proteins factor 2 isoform 1) c4( ) c5(180378, 180379, 180380, 180381, 180382, 180383, 180384, 180385) c6( ) c7(T, cqm, eH, ml, cqn, K, nA, eF, cql, dr); #c1(PGAP2) c2(XP_006718246) c3(post-GPI attachment to proteins factor 2 isoform X14) c4( ) c5(291651) c6( ) c7(T, cqm, eH, ml, con, K, nA, eF, cql, dr); #c1(PGAP2) c2(XP_006718248) c3(post-GPI attachment to proteins factor 2 isoform X15) c4( ) c5(291652) c6( ) c7(T, com, eH, ml, con, K, nA, eF, cql, dr); #c1(PGAP2) c2(XP_011518296) c3(post-GPI attachment to proteins factor 2 isoform X8) c4(312324) c5(312325, 312326, 312327, 312328, 312329, 312330) c6( ) c7(T, cqm, eH, ml, cqn, K, nA, eF, cql, dr); #c1(PGAP2) c2(XP_011518310) c3(post-GPI attachment to proteins factor 2 isoform X17) c4(312331) c5(312332, 312333, 312334, 312335, 312336) c6( ) c7(T, com, eH, ml, cqn, K, nA, eF, cql, dr); #c1(PGAP3) c2(NP_001278655) c3(post-GPI attachment to proteins factor 3 isoform 2 precursor) c4(109949) c5(109950, 109951, 109952, 109953, 109954, 109955) c6( ) c7(bL, T, eR, e, al, bzb, jN, aA, arD, cz, aj, bS, Jd, ct, cql, nA, dG, j, v, qj, q, J, aZE, ad, vm, fU, cqo, aaF, AW, XW, aE, Na, nv, sO, aT, h, aNU, bK, K, con, he, Xb, ae, fb, Gc, je, aiV, aw, gT, aW, hl, r, nf, agG, yy, bxx, bMC, cl, Z, f, ik, Ym, bz, CQ, bN, asE, ck, apM, iM, eM, bE, dc, wE, ZD, hR, uz, Km, dy, ei, bD, pJ, B, y, pC); #c1(PGAP3) c2(NP_001278657) c3(post-GPI attachment to proteins factor 3 isoform 3 precursor) c4(109956) c5(109957, 109958, 109959, 109960, 109961, 109962) c6( ) c7(bL, T, eR, e, al, bzb, jN, aA, arD, cz, aj, bS, Jd, ct, cql, nA, dG, j, v, qj, q, J, aZE, ad, vm, fU, cqo, aaF, AW, XW, aE, Na, nv, sO, aT, h, aNU, bK, K, con, he, Xb, ae, fb, Gc, je, aiV, aw, gT, aW, hl, r, nf, agG, yy, bxx, bMC, cl, Z, f, iK, Ym, bz, CQ, bN, asE, ck, apM, iM, eM, bE, dc, wE, ZD, hR, uz, Km, dy, ei, bD, pJ, B, y, pC); #c1(PGAP3) c2(NP_219487) c3(post-GPI attachment to proteins factor 3 isoform 1 precursor) c4(228574, 228575) c5(228576, 228577, 228578, 228579, 228580, 228581, 228582, 228583, 228584, 228585, 228586, 228587) c6( ) c7(bL, T, eR, e, al, bzb, jN, aA, arD, cz, aj, bS, Jd, ct, cql, nA, dG, j, v, qj, q, J, aZE, ad, vm, fU, cqo, aaF, AW, XW, aE, Na, nv, sO, aT, h, aNU, bK, K, cqn, he, Xb, ae, fb, Gc, je, aiV, aw, gT, aW, hl, r, nf, agG, yy, bxx, bMC, cl, Z, f, ik, Ym, bz, CQ, bN, asE, ck, apM, iM, eM, bE, dc, wE, ZD, hR, uz, Km, dy, ei, bD, pJ, B, y, pC); #c1(PGBD1) c2(NP_001171672) c3 (piggyBac transposable element-derived protein 1) c4(71264) c5(71265, 71266, 71267, 71268, 71269, 71270, 71271, 71272, 71273, 71274) c6( ) c7(af, eZ); #c1(PGBD5) c2(NP_001245240) c3(piggyBac transposable element-derived protein 5) c4(92779, 92780) c5(92781, 92782, 92783, 92784, 92785, 92786, 92787, 92788, 92789, 92790, 92791, 92792) c6( ) c7(aw, fq, alv); #c1(PGC) c2(NP_001159896) c3(gastricsin isoform 2 preproprotein) c4(64931, 64932) c5(64933, 64934, 64935, 64936, 64937, 64938, 64939, 64940, 64941, 64942, 64943, 64944, 64945, 64946, 64947, 64948, 64949) c6( ) c7(bL, T, cqp, wu, aZ, aA, cz, aj, dG, v, ci, ml, fr, q, J, ad, cc, qp, aT, bK, K, wm, bl, ae, af, dl, A, vT, Jl, aW, Y, az, eH, fc, r, Mz, bEX, aq, aF, aB, kt, bp, Sm, eF, zE, ve, qa, fT, rR, iA, mx, U, gZ, mf, cP, ei, QV, B, aJC, L); #c1(PGC) c2(NP_002621) c3(gastricsin isoform 1 preproprotein) c4(130367, 130368) c5(130369, 130370, 130371, 130372, 130373, 130374, 130375) c6( ) c7(bL, T, cqp, wu, aZ, aA, cz, aj, dG, v, ci, ml, fr, q, J, ad, cc, qp, aT, bK, K, wm, bl, ae, af, dl, A, vT, Jl, aW, Y, az, eH, fc, r, Mz, bEX, aq, aF, aB, kt, bp, Sm, eF, zE, ve, qa, fT, rR, iA, mx, U, gZ, mf, cP, ei, QV, B, aJC, L); #c1(PGD) c2(NP_001291380) c3(6-phosphogluconate dehydrogenase, decarboxylating isoform 2) c4(115815) c5(115816, 115817, 115818, 115819, 115820, 115821) c6( ) c7(Nq, bM, T, Nr, BU, Uu, Ew, dV, vH, aq, Xm, jE, cqq, aTS, f, jT, Ku, eQ, B, dR); #c1(PGD) c2(NP_001291381) c3(6-phosphogluconate dehydrogenase, decarboxylating isoform 3) c4( ) c5(115822, 115823, 115824, 115825, 115826, 115827, 115828, 115829, 115830, 115831, 115832) c6( ) c7(Nq, bM, T, Nr, BU, Uu, Ew, dV, vH, aq, Xm, jE, cqq, aTS, f, jT, Ku, eQ, B, dR); #c1(PGF) c2(NP_001193941) c3 (placenta growth factor isoform 2 precursor) c4(83270, 83271) c5(83272, 83273, 83274, 83275, 83276, 83277, 83278, 83279, 83280, 83281, 83282, 83283) c6( ) c7(T, ea, e, al, eb, aY, ip, x, Vp, K, aiJ, hm, DA, yo, Au, g, uC, QA, eM, nB, AM, wE, aBO, vy, dD, aw, aj, cd, ai, ft, gd, aze, fQ, aq, cB, bL, eR, jx, rf, cz, cR, j, q, J, Rl, uT, cy, bq, ae, vT, Y, blq, r, en, bu, f, c, dh, fD, rA, bt, ao, hS, sY, Pt, qb, eq, ad, hX, dP, av, hd, py, lC, oW, nJ, azH, d, ff, yy, bz, gk, pc, zr, bE, y); #c1(PGF) c2(NP_002623) c3(placenta growth factor isoform 1 precursor) c4(130376) c5(130377, 130378, 130379, 130380, 130381, 130382) c6( ) c7(T, ea, e, al, eb, aY, ip, x, Vp, K, aiJ, hm, DA, yo, Au, g, uC, QA, eM, nB, AM, wE, aBO, vy, dD, aw, aj, cd, ai, ft, gd, aze, fQ, aq, cB, bL, eR, jx, rf, cz, cR, j, q, J, Rl, uT, cy, bq, ae, vT, Y, blq, r, en, bu, f, c, dh, fD, rA, bt, ao, hS, sY, Pt, qb, eq, ad, hX, dP, av, hd, py, lC, oW, nJ, azH, d, ff, yy, bz, gk, pc, zr, bE, y); #c1(PGGT1B) c2(NP_005014) c3(geranylgeranyl transferase type-1 subunit beta) c4(152172) c5(152173, 152174, 152175, 152176, 152177, 152178) c6( ) c7(zu, ey, Da, pb, aE); #c1(PGGT1B) c2(XP_005272079) c3(geranylgeranyl transferase type-1 subunit beta isoform X5) c4(283667) c5(283668, 283669, 283670, 283671, 283672, 283673) c6( ) c7(zu, ey, Da, pb, aE); #c1(PGK1) c2(NP_000282) c3(phosphoglycerate kinase 1) c4(6142, 6143, 6144, 6145, 6146) c5(6147, 6148, 6149, 6150, 6151, 6152, 6153, 6154, 6155, 6156, 6157, 6158, 6159, 6160, 6161, 6162, 6163, 6164, 6165, 6166, 6167, 6168, 6169, 6170) c6( ) c7(T, bRZ, aA, bB, fr, ci, nA, ad, bjA, ep, oW, aT, aVg, ae, xX, B, bRX, aW, Y, PX, aNq, aq, kp, aF, BG, cm, bw, dr, bRW, yd, eM, bE, X); #c1(PGK2) c2(NP_620061) c3(phosphoglycerate kinase 2) c4(237240, 237241) c5(237242, 237243, 237244, 237245, 237246, 237247) c6( ) c7(S); #c1(PGLS) c2(NP_036220) c3(6-phosphogluconolactonase) c4(172432) c5(172433, 172434, 172435, 172436, 172437, (72438) c6( ) c7(T, bA); #c1(PGLYRP1) c2(NP_005082) c3(peptidoglycan recognition protein 1 precursor) c4(152724, 152725, 152726) c5(152727, 152728, 152729, 152730, 152731, 152732, 152733, 152734) c6(152735, 152736) c7(fr, wR, aFi, Y, cr); #c1(PGLYRP2) c2(NP_443122) c3(N-acetylmuramoyl-L-alanine amidase precursor) c4(229554) c5(229555, 229556, 229557, 229558, 229559, 229560, 229561, 229562) c6(229563, 229564) c7(oO, ka, ac, ki, e, ht, aw, ff, j, en, eu); #c1(PGLYRP2) c2(XP_006722696) c3(N-acetylmuramoyl-L-alanine amidase isoform X1) c4(294832) c5(294833, 294834, 294835, 294836, 294837, 294838, 294839, 294840, 294841, 294842) c6(294843, 294844, 294845) c7(oO, ka, ac, ki, e, ht, aw, ff, j, en, eu); #c1(PGLYRP3) c2(XP_011507421) c3(peptidoglycan recognition protein 3 isoform X1) c4(297338) c5(297339, 297340, 297341, 297342, 297343, 297344, 297345, 297346) c6(297347, 297348) c7(eE, cr, Xk, Ni); #c1(PGLYRP4) c2(NP_065126) c3(peptidoglycan recognition protein 4 precursor) c4(204067) c5(204068, 204069, 204070, 204071, 204072, 204073, 204074, 204075) c6(204076, 204077) c7(cr, Xk); #c1(PGLYRP4) c2(XP_011508093) c3 (peptidoglycan recognition protein 4 isoform X2) c4(298578) c5(298579, 298580, 298581, 298582, 298583, 298584, 298585, 298586) c6(298587, 298588) c7(cr, Xk); #c1(PGM3) c2(NP_001186848) c3(phosphoacetylglucosamine mutase isoform 4) c4(78702, 78703) c5(78704, 78705, 78706, 78707, 78708, 78709) c6( ) c7(Km); #c1(PGM3) c2(NP_056414) c3(phosphoacetylglucosamine mutase isoform 2) c4(187498) c5(187499, 187500, 187501, 187502, 187503) c6( ) c7(Km); #c1(PGM3) c2(XP_011534205) c3(phosphoacetylglucosamine mutase isoform X1) c4(330513) c5(330514, 330515, 330516, 330517, 330518, 330519, 330520, 330521, 330522, 330523, 330524, 330525, 330526) c6( ) c7(Km); #c1(PGP) c2(NP_001035830) c3 (phosphoglycolate phosphatase) c4(33197) c5(33198, 33199, 33200, 33201, 33202, 33203) c6( ) c7(iT, wB, fe, hn, bK, hB, mY, bt); #c1(PGPEP1) c2(NP_060182) c3 (pyroglutamyl-peptidase 1 isoform 1) c4(194634, 194635) c5(194636, 194637, 194638, 194639, 194640, 194641, 194642) c6( ) c7(T, iT, lp, dc, hB, dO, ask, aT, bt, gl, h, he, j, Jz, hn, fD, fe, anN, V); #c1(PGPEP1) c2(XP_011526417) c3 (pyroglutamyl-peptidase 1 isoform X6) c4( ) c5(320722, 320723, 320724, 320725, 320726) c6( ) c7(T, iT, lp, dc, hB, dO, ask, aT, bt, gl, h, he, j, Jz, hn, fD, fe, anN, V); #c1(PGR) c2(NP_000917) c3(progesterone receptor isoform B) c4(17170, 17171, 17172, 17173) c5(17174, 17175, 17176, 17177, 17/78, 17179, 17180) c6( ) c7(T, fR, pf, Xi, ci, ip, lS, aE, il, pX, aeO, iv, K, bU, wt, xQ, zQ, aeR, aW, g, as, uC, ajj, eM, AM, vA, uo, cX, m, Th, ik, sQ, wu, sw, zX, qy, n, ww, ml, zP, hw, pN, UD, dL, hB, mJ, aiB, MD, mo, PU, cM, auC, op, Ye, dB, hL, bm, at, ut, aEc, ZZ, adG, fM, aA, v, AO, q, er, asG, aHu, aew, BJ, aT, ae, bA, qw, avg, kl, s, ND, NE, Y, LO, r, eS, en, cl, apx, t, Px, ck, sn, kP, Nk, bt, gx, nL, aZ, iW, uu, ad, dP, avp, ib, vJ, aRD, io, lQ, wm, nJ, WZ, auY, d, tb, qt, PE, ff, NH, aF, bz, es, aoR, ag, auZ, y, aed); #c1 (PGR) c2(NP_001189403) c3(progesterone receptor isoform A) c4( ) c5(79365) c6( ) c7(T, FR, pf, Xi, ci, ip, lS, aE, il, pX, aeO, iv, K, bU, wt, xl, zQ, aeR, aW, g, as, uC, ajj, eM, AM, vA, uo, cX, m, Th, ik, sQ, wu, sw, zX, qy, n, ww, ml, zP, hw, pN, UD, dL, hB, mJ, aiB, MD, mo, PU, cM, auC, op, Ye, dB, hL, bm, at, ut, aEc, ZZ, adG, fM, aA, v, AO, q, er, asG, aHu, aew, BJ, aT, ae, bA, qw, avg, kl, s, ND, NE, Y, LO, r, eS, en, cl, apx, t, Px, ck, sn, kP, Nk, bt, gx, nL, aZ, iW, uu, ad, dP, avp, ib, vJ, aRD, io, lQ, wm, nJ, WZ, auY, d, tb, qt, PE, ff, NH, aF, bz, es, aoR, ag, auZ, y, aed); #c1(PGR) c2(NP_001258090) c3(progesterone receptor isoform C) c4(95945) c5(95946, 95947, 95948, 95949, 95950, 95951) c6( ) c7(T, FR, pf, Xi, ci, ip, lS, aE, il, pX, aeO, iv, K, bU, wt, xQ, zQ, aeR, aW, g, as, uC, aj, eM, AM, vA, uo, cX, m, Th, ik, sQ, wu, sw, zX, qy, n, ww, ml, zP, hw, pN, UD, dL, hB, mJ, aiB, MD, mo, PU, cM, auC, op, Ye, dB, hL, bm, at, ut, aEc, ZZ, adG, fM, aA, v, AO, q, er, asG, aHu, aew, BJ, aT, ae, bA, qw, avg, kl, s, ND, NE, Y, LO, r, eS, en, cl, apx, t, Px, ck, sn, kP, Nk, bt, gx, nl, aZ, iW, uu, ad, dP, avp, ib, vJ, aRD, io, lQ, wm, nJ, WZ, auY, d, tb, qt, PE, ff, NH, af, bz, es, aoR, ag, auZ, y, aed); #c1(PGR) c2(NP_001258091) c3(progesterone receptor isoform D) c4( ) c5(95952, 95953, 95954, 95955, 95956) c6( ) c7(T, fR, pf, Xi, ci, ip, lS, aE, il, pX, aeO, iv, K, bU, wt, xQ, zQ, aeR, aW, g, as, uC, ajj, eM, AM, vA, uo, cx, m, Th, ik, sQ, wu, sw, zX, qy, n, ww, ml, zP, hw, pN, UD, dL, hB, mJ, aiB, MD, mo, PU, cM, auC, op, Ye, dB, hL, bm, at, ut, aEc, ZZ, adG, fM, aA, v, AO, q, er, asG, aHu, aew, BJ, aT, ae, bA, qw, avg, kl, s, ND, NE, Y, LO, r, eS, en, cl, apx, t, Px, ck, sn, kP, Nk, bt, gx, nl, aZ, iW, uu, ad, dP, avp, ib, vJ, aRD, io, lQ, wm, nJ, WZ, auY, d, tb, qt, PE, ff, NH, aF, bz, es, aoR, ag, auZ, y, aed); #c1(PGR) c2(XP_006718921) c3(progesterone receptor isoform X1) c4( ) c5(292088, 292089, 292090, 292091, 292092, 292093, 292094, 292095, 292096, 292097, 292098, 292099, 292100, 292101, 292102, 292103, 292104, 292105, 292106, 292107, 292108, 292109, 292110, 292111) c6( ) c7(T, fR, pf, Xi, ci, ip, lS, aE, il, pX, aeO, iv, K, bU, wt, xQ, zQ, aeR, aW, g, as, uC, ajj, eM, AM, vA, uo, cX, m, Th, ik, sQ, wu, sw, zX, qy, n, ww, ml, zP, hw, pN, UD, dL, hB, mJ, aiB, MD, mo, PU, cM, auC, op, Ye, dB, hL, bm, at, ut, aEc, ZZ, adG, fM, aA, v, AO, q, er, asG, aHu, aew, BJ, aT, ae, bA, qw, avg, kl, s, ND, NE, Y, LO, r, eS, en, cl, apx, t, Px, ck, sn, kP, Nk, bt, gx, nl, aZ, iW, uu, ad, dP, avp, ib, vJ, aRD, io, lQ, wm, nJ, WZ, auY, d, tb, qt, PE, ff, NH, aF, bz, es, aoR, ag, auZ, y, aed); #c1(PGR) c2(XP_011541171) c3(progesterone receptor isoform X2) c4(339762, 339763) c5(339764, 339765, 339766, 339767) c6( ) c7(T, FR, pf, Xi, ci, ip, lS, aE, il, pX, aeO, iv, K, bU, wt, xQ, zQ, aeR, aW, g, as, uC, ajj, eM, AM, vA, uo, cx, m, Th, ik, sQ, wu, sw, zX, qy, n, ww, ml, zP, hw, pN, UD, dL, hB, mJ, aiB, MD, mo, PU, cM, auC, op, Ye, dB, hL, bm, at, ut, aEc, ZZ, adG, fM, aA, v, AO, q, er, asG, aHu, aew, BJ, aT, ae, bA, qw, avg, kl, s, ND, NE, Y, LO, r, eS, en, cl, apx, t, Px, ck, sn, kP, Nk, bt, gx, nL, aZ, iW, uu, ad, dP, avp, ib, vJ, aRD, io, lQ, wm, nJ, WZ, auY, d, tb, qt, PE, ff, NH, aF, bz, es, aoR, ag, auZ, y, aed); #c1(PGRMC1) c2(NP_001269550) c3(membrane-associated progesterone receptor component 1 isoform 2) c4(101894) c5(101895, 101896, 101897, 101898, 101899, 101900) c6( ) c7(T, fR, sQ, if, qN, ad, b, il, dV, aT, ey, lQ, K, aW, g, zZ, t, bz, Rd, uo, ut, m, i); #c1(PGRMC1) c2(NP_006658) c3(membrane-associated progesterone receptor component 1 isoform 1) c4(166965) c5(166966, 166967, 166968, 166969, 166970, 166971) c6( ) c7(T, fR, sQ, if, qN, ad, b, il, dV, aT, ey, lQ, K, aW, g, zZ, t, bz, Rd, uo, ut, m, i); #c1(PGRMC2) c2(NP_006311) c3(membrane-associated progesterone receptor component 2) c4(163899, 163900) c5(163901, 163902, 163903, 163904, 163905, 163906, 163907, 163908, 163909, 163910, 163911, 163912, 163913, 163914) c6( ) c7(BN, fR, sQ, cz, uo, ut, hy, lQ, ne); #c1(PGRMC2) c2(XP_011529835) c3(membrane-associated progesterone receptor component 2 isoform X1) c4(325636) c5(325637, 325638, 325639) c6( ) c7(BN, fR, sQ, cz, uo, ut, hy, lQ, ne); #c1(PHACTR1) c2(NP_001229577) c3(phosphatase and actin regulator 1) c4(84676, 84677, 84678, 84679) c5(84680, 84681, 84682, 84683, 84684, 84685, 84686, 84687, 84688, 84689, 84690, 84691, 84692, 84693, 84694, 84695, 84696, 84697, 84698) c6( ) c7(cqr, aW, aw, aT, j, pT, vo, eN); #c1(PHACTR1) c2(XP_005248994) c3(phosphatase and actin regulator 1 isoform X5) c4( ) c5(268816, 268817, 268818, 268819, 268820, 268821, 268822, 268823, 268824, 268825, 268826, 268827, 268828, 268829, 268830, 268831, 268832, 268833, 268834, 268835, 268836, 268837, 268838) c6( ) c7(cqr, aW, aw, aT, j, pT, vo, eN); #c1(PHACTR1) c2(XP_011512692) c3(phosphatase and actin regulator 1 isoform X1) c4(303697, 303698, 303699, 303700, 303701) c5(303702, 303703, 303704, 303705, 303706, 303707, 303708, 303709, 303710, 303711, 303712, 303713) c6( ) c7(cqr, aW, aw, aT, j, pT, vo, eN); #c1(PHACTR1) c2(XP_011512693) c3(phosphatase and actin regulator 1 isoform X2) c4(303714, 303715) c5(303716) c6( ) c7(cqr, aW, aw, aT, j, pT, vo, eN); #c1(PHACTR1) c2(XP_011512694) c3(phosphatase and actin regulator 1 isoform X3) c4( ) c5(303717, 303718, 303719, 303720, 303721, 303722, 303723, 303724, 303725, 303726, 303727) c6( ) c7(cqr, aW, aw, aT, j, pT, vo, eN); #c1(PHACTR2) c2(NP_001093634) c3(phosphatase and actin regulator 2 isoform 1) c4(40364) c5(40365, 40366, 40367, 40368, 40369, 40370) c6( ) c7(ac, pN, aw, an, V, af); #c1(PHACTR2) c2(NP_001093635) c3(phosphatase and actin regulator 2 isoform 2) c4(40371, 40372) c5(40373, 40374, 40375, 40376, 40377, 40378, 40379, 40380, 40381, 40382, 40383) c6( ) c7(ac, pN, aw, an, V, af); #c1(PHACTR2) c2(NP_001093636) c3(phosphatase and actin regulator 2 isoform 4) c4(40384) c5(40385, 40386, 40387, 40388, 40389, 40390, 40391) c6( ) c7(ac, pN, aw, an, V, af); #c1(PHACTR2) c2(NP_055536) c3(phosphatase and actin regulator 2 isoform 3) c4(181819) c5(181820, 181821, 181822, 181823, 181824, 181825) c6( ) c7(ac, pN, aw, an, V, af); #c1(PHACTR3) c2(NP_001186434) c3(phosphatase and actin regulator 3 isoform 4) c4( ) c5(77637, 77638, 77639) c6( ) c7(aw, oe, r, ci, y, q); #c1(PHACTR3) c2(NP_542403) c3(phosphatase and actin regulator 3 isoform 1) c4(232373, 232374) c5(232375, 232376, 232377, 232378, 232379, 232380, 232381, 232382, 232383, 232384, 232385) c6( ) c7(aw, oe, r, ci, y, q); #c1(PHACTR3) c2(NP_899069) c3(phosphatase and actin regulator 3 isoform 3) c4( ) c5(257953, 257954, 257955, 257956, 257957, 257958, 257959, 257960) c6( ) c7(aw, oe, r, ci, y, q); #c1(PHACTR3) c2(XP_011526827) c3(phosphatase and actin regulator 3 isoform X1) c4( ) c5(321175, 321176, 321177, 321178, 321179, 321180, 321181, 321182, 321183, 321184, 321185) c6( ) c7(aw, oe, r, ci, y, q); #c1(PHACTR3) c2(XP_011526828) c3(phosphatase and actin regulator 3 isoform X2) c4(321186, 321187) c5(321188, 321189, 321190) c6( ) c7(aw, oe, r, ci, y, q); #c1(PHB) c2(NP_001268426) c3(prohibitin isoform 2) c4(99910) c5(99911, 99912, 99913, 99914, 99915, 99916, 99917, 99918, 99919, 99920, 99921, 99922) c6( ) c7(T, AS, ht, aA, aj, v, ci, zP, ad, J, hd, Db, ju, aT, dL, bK, K, ji, ae, zQ, aW, fe, aq, aF, g, ir, t, aDQ, qJ, nB, U, at, sv, aDR, m, it, B); #c1(PHB) c2(NP_002625) c3(prohibitin isoform 1) c4(130383, 130384, 130385, 130386, 130387, 130388) c5(130389, 130390, 130391, 130392, 130393, 130394, 130395, 130396, 130397, 130398, 130399, 130400, 130401, 130402, 130403, 130404, 130405, 130406, 130407, 130408, 130409, 130410, 130411, 130412, 130413, 130414, 130415, 130416, 130417, 130418) c6( ) c7(T, AS, ht, aA, aj, v, ci, zP, ad, J, hd, Db, ju, aT, dL, bK, K, ji, ae, zQ, aW, fe, aq, aF, g, ir, t, aDQ, qJ, nB, U, at, sv, aDR, m, it, B); #c1(PHB2) c2(NP_001138303) c3(prohibitin-2 isoform 1) c4(56142, 56143) c5(56144, 56145, 56146, 56147, 56148, 56149, 56150, 56151, 56152, 56153, 56154, 56155, 56156) c6( ) c7(nJ, bv, T, bdQ, aNB, cqs, DK, nv, aW, rz, vT, Pg, aT, aA, n, aF, H, Xb, B, bN, L); #c1(PHB2) c2(NP_001254629) c3(prohibitin-2 isoform 3) c4(94720, 94721) c5(94722, 94723, 94724, 94725, 94726, 94727, 94728, 94729, 94730, 94731, 94732, 94733, 94734, 94735) c6( ) c7(nJ, bv, T, bdQ, aNB, cqs, DK, nv, aW, rz, vT, Pg, aT, aA, n, aF, H, Xb, B, bN, L); #c1(PHC1) c2(NP_004417) c3(polyhomeotic-like protein 1) c4(146218, 146219) c5(146220, 146221, 146222, 146223, 146224, 146225) c6( ) c7(ck, x, Mg, cqt, z, Lb, k, eA, bD, aH, aij); #c1(PHC1) c2(XP_011518901) c3 (polyhomeotic-like protein 1 isoform X2) c4(313262) c5( ) c6( ) c7(ck, x, Mg, cqt, z, Lb, k, eA, bD, aH, aij); #c1(PHC1) c2(XP_011518904) c3(polyhomeotic-like protein 1 isoform X6) c4( ) c5(313263, 313264, 313265, 313266, 313267, 313268, 313269, 313270, 313271, 313272, 313273, 313274, 313275, 313276, 313277, 313278, 313279, 313280, 313281, 313282, 313283) c6( ) c7(ck, x, Mg, cqt, z, Lb, k, eA, bD, aH, aij); #c1(PHC2) c2(XP_011539177) c3(polyhomeotic-like protein 2 isoform X3) c4( ) c5(337321, 337322, 337323, 337324, 337325) c6( ) c7(bkT, wS, ad); #c1(PHC3) c2(NP_079223) c3(polyhomeotic-like protein 3) c4(217497, 217498, 217499, 217500) c5(217501, 217502, 217503, 217504, 217505, 217506, 217507, 217508, 217509, 217510, 217511, 217512) c6( ) c7(eF, T, eH, ci); #c1(PHC3) c2(XP_006713819) c3(polyhomeotic-like protein 3 isoform X7) c4(287386, 287387) c5(287388, 287389, 287390, 287391, 287392, 287393) c6( ) c7(eF, T, eH, ci); #c1(PHC3) c2(XP_006713820) c3(polyhomeotic-like protein 3 isoform X11) c4( ) c5(287394, 287395, 287396, 287397, 287398, 287399, 287400, 287401, 287402, 287403, 287404, 287405, 287406, 287407, 287408, 287409, 287410, 287411) c6( ) c7(eF, T, eH, ci); #c1(PHC3) c2(XP_011511489) c3(polyhomeotic-like protein 3 isoform X5) c4(302168) c5(302169, 302170, 302171, 302172, 302173) c6( ) c7(eF, T, eH, ci); #c1(PHEX) c2(NP_000435) c3(phosphate-regulating neutral endopeptidase isoform 1) c4(8957, 8958, 8959) c5(8960, 8961, 8962, 8963, 8964, 8965, 8966, 8967, 8968, 8969, 8970, 8971) c6( ) c7(bL, cqu, bgv, AB, qb, ha, Ay, cz, amC, q, eA, Si, amB, Tl, bgm, hP, mL, ji, af, aT, ajx, vj, Rj, Az, eH, awu, eF, bfH, AA, nC, aBU, mE, lO, ajY, aT, azg, bfl, LR, zA, ars); #c1(PHEX) c2(NP_001269683) c3(phosphate-regulating neutral endopeptidase isoform 2) c4(102543) c5(102544, 102545, 102546, 102547, 102548, 102549) c6( ) c7(bL, cqu, bgv, AB, qb, ha, Ay, cz, amC, q, eA, Si, amB, Tl, bgm, hP, mL, ji, af, aT, ajx, vj, Rj, Az, eH, awu, eF, bfH, AA, nC, aBU, mE, lO, ajY, aL, azg, bfl, LR, zA, ars); #c1(PHEX) c2(XP_011543836) c3(phosphate-regulating neutral endopeptidase isoform X1) c4( ) c5(342980, 342981, 342982, 342983, 342984, 342985) c6( ) c7(bL, cqu, bgv, AB, qb, ha, Ay, cz, amC, q, eA, Si, amB, Tl, bgm, hP, mL, ji, af, al, ajx, vj, Rj, Az, eH, awu, eF, bfH, AA, nC, aBU, mE, lO, ajY, aL, azg, bfl, LR, ZA, ars); #c1(PHF1) c2(NP_002627) c3(PHD finger protein 1 isoform a) c4(130438, 130439) c5(130440, 130441, 130442, 130443, 130444, 130445, 130446, 130447, 130448, 130449, 130450, 130451) c6( ) c7(T, aW, aEm, eZ, aT, cqv, Th, aaO, bmq, af); #c1(PHF1) c2(XP_011512965) c3(PHD finger protein 1 isoform X1) c4( ) c5(304395) c6( ) c7(T, aW, aEm, eZ, aT, cqv, Th, aaO, bmq, af); #c1(PHF1) c2(XP_011512968) c3(PHD finger protein 1 isoform X4) c4( ) c5(304396) c6( ) c7(T, aW, aEm, eZ, aT, cqv, Th, aal, bmq, af); #c1(PHF1) c2(XP_011512970) c3(PHD finger protein 1 isoform X5) c4(304397, 304398) c5(304399, 304400, 304401, 304402, 304403, 304404, 304405) c6( ) c7(T, aW, aEm, eZ, aT, cqv, Th, aaO, bmq, af); #c1(PHF1) c2(XP_011512972) c3 (PHD finger protein 1 isoform X7) c4( ) c5(304406, 304407, 304408, 304409, 304410, 304411, 304412, 304413, 304414, 304415, 304416, 304417, 304418, 304419, 304420, 304421, 304422, 304423) c6( ) c7(T, aW, aEm, eZ, aT, cqv, Th, aaO, bmq, af); #c1(PHF1) c2(XP_011512973) c3(PHD finger protein 1 isoform X8) c4( ) c5(304424, 304425, 304426, 304427, 304428, 304429, 304430, 304431, 304432, 304433) c6( ) c7(T, aW, aEm, eZ, aT, cqv, Th, aaO, bmq, af); #c1(PHF1) c2(XP_011546746) c3(PHD finger protein 1 isoform X2) c4(345662) c5( ) c6( ) c7(T, aW, aEm, eZ, aT, cqv, Th, aal, bmq, af); #c1(PHF1) c2(XP_011546750) c3(PHD finger protein 1 isoform X5) c4(345663) c5(345664, 345665, 345666, 345667, 345668, 345669) c6( ) c7(T, aW, aEm, eZ, aT, cqv, Th, aaO, bmq, af); #c1(PHF1) c2(XP_011546753) c3(PHD finger protein 1 isoform X8) c4(345670, 345671) c5( ) c6( ) c7(T, aW, aEm, eZ, aT, cqv, Th, aaO, bmq, af); #c1(PHF10) c2(NP_060758) c3(PHD finger protein 10 isoform a) c4(197642) c5(197643, 197644, 197645, 197646, 197647, 197648) c6( ) c7(afz, aF, aA); #c1(PHF10) c2(NP_579866) c3(PHD finger protein 10 isoform b) c4(234669) c5(234670, 234671, 234672, 234673, 234674, 234675) c6( ) c7(afz, af, aA); #c1(PHF11) c2(NP_001035533) c3(PHD finger protein 11 isoform a) c4(32867, 32868, 32869, 32870, 32871) c5(32872, 32873, 32874, 32875, 32876, 32877, 32878, 32879) c6( ) c7(buV, bM, Dw, z, sp, oe, mQ, eE, da, Ds, alO, k, bGD); #c1(PHF11) c2(XP_006719891) c3(PHD finger protein 11 isoform X2) c4( ) c5(293057, 293058) c6( ) c7(buV, bM, Dw, z, sp, oe, mQ, eE, da, Ds, alO, k, bGD); #c1(PHF11) c2(XP_011533404) c3(PHD finger protein 11 isoform X1) c4( ) c5(329481, 329482, 329483, 329484, 329485, 329486, 329487, 329488, 329489, 329490, 329491, 329492, 329493, 329494, 329495, 329496, 329497, 329498, 329499, 329500, 329501, 329502, 329503, 329504) c6( ) c7(buV, bM, Dw, z, sp, oe, mQ, eE, da, Ds, alO, k, bGD); #c1(PHF12) c2(NP_001028733) c3(PHD finger protein 12 isoform 1) c4(30582) c5(30583, 30584, 30585, 30586, 30587, 30588) c6( ) c7(arD, af); #c1(PHF12) c2(NP_001277060) c3(PHD finger protein 12 isoform 3) c4(108997) c5(108998, 108999, 109000, 109001, 109002, 109003) c6( ) c7(arD, af); #c1(PHF12) c2(NP_065940) c3(PHD finger protein 12 isoform 2) c4(206340) c5(206341, 206342, 206343, 206344, 206345, 206346, 206347, 206348, 206349, 206350) c6( ) c7(arD, af); #c1(PHF19) c2(NP_001009936) c3(PHD finger protein 19 isoform b) c4(23744, 23745) c5(23746, 23747, 23748, 23749, 23750, 23751, 23752, 23753, 23754, 23755, 23756) c6( ) c7(y, Y); #c1(PHF19) c2(NP_001273769) c3(PHD finger protein 19 isoform c) c4(105843, 105844) c5(105845, 105846, 105847, 105848, 105849, 105850, 105851, 105852, 105853, 105854, 105855, 105856) c6( ) c7(y, Y); #c1(PHF19) c2(NP_001273771) c3(PHD finger protein 19 isoform d) c4( ) c5(105865, 105866, 105867, 105868, 105869) c6( ) c7(y, Y); #c1(PHF19) c2(NP_001273772) c3(PHD finger protein 19 isoform e) c4( ) c5(105870, 105871, 105872, 105873, 105874) c6( ) c7(y, Y); #c1(PHF19) c2(NP_056466) c3(PHD finger protein 19 isoform a) c4(187645) c5( ) c6( ) c7(y, Y); #c1(PHF19) c2(XP_006717109) c3(PHD finger protein 19 isoform X6) c4( ) c5(290733) c6( ) c7(y, Y); #c1(PHF19) c2(XP_011516816) c3(PHD finger protein 19 isoform X4) c4(310260) c5( ) c6( ) c7(y, Y); #c1(PHF19) c2(XP_011516818) c3(PHD finger protein 19 isoform X7) c4( ) c5(310261, 310262) c6( ) c7(y, Y); #c1(PHF2) c2(NP_005383) c3(lysine-specific demethylase PHF2) c4(155589) c5(155590, 155591, 155592, 155593, 155594, 155595) c6( ) c7(aW, aT); #c1(PHF2) c2(XP_0067/7206) c3(lysine-specific demethylase PHF2 isoform X3) c4( ) c5(290806, 290807, 290808, 290809, 290810, 290811) c6( ) c7(aW, aT); #c1(PHF2) c2(XP_011517086) c3(lysine-specific demethylase PHF2 isoform X2) c4(310612) c5( ) c6( ) c7(aW, aT); #c1(PHF20) c2(NP_057520) c3(PHD finger protein 20) c4(191216, 191217, 191218, 191219) c5(191220, 191221, 191222, 191223, 191224, 191225, 191226, 191227, 191228, 191229, 191230, 191231, 191232, 191233, 191234, 191235, 191236, 191237, 191238) c6( ) c7(T, hd, d, s, eM, kH, m, as, t, iK, bz, eA); #c1(PHF20) c2(XP_011527144) c3(PHD finger protein 20 isoform X1) c4( ) c5(321658, 321659, 321660, 321661, 321662) c6( ) c7(T, hd, d, s, eM, kH, m, as, t, iK, bz, eA); #c1(PHF21A) c2(NP_001095272) c3(PHD finger protein 21A isoform a) c4(40854, 40855, 40856) c5(40857, 40858, 40859, 40860, 40861, 40862) c6( ) c7(ac, af, Az, nA); #c1(PHF21A) c2(NP_057705) c3(PHD finger protein 21A isoform b) c4(192455) c5(192456, 192457, 192458, 192459, 192460, 192461) c6( ) c7(ac, af, Az, nA); #c1(PHF21A) c2(XP_005253022) c3(PHD finger protein 21A isoform X5) c4(272107) c5(272108, 272109, 272110, 272111, 272112, 272113) c6( ) c7(ac, af, Az, nA); #c1(PHF21A) c2(XP_011518472) c3(PHD finger protein 21A isoform X2) c4( ) c5(312532, 312533, 312534, 312535, 312536, 312537, 312538) c6( ) c7(ac, af, Az, nA); #c1(PHF21A) c2(XP_011518483) c3(PHD finger protein 21A isoform X12) c4( ) c5(312539, 312540, 312541, 312542, 312543, 312544, 312545, 312546, 312547, 312548, 312549, 312550) c6( ) c7(ac, af, Az, nA); #c1(PHFZlA) c2(XP_011518484) c3(PHD finger protein 21A isoform X13) c4(312551) c5(312552) c6( ) c7(ac, af, Az, nA); #c1(PHF23) c2(NP_001271447) c3(PHD finger protein 23 isoform 3) c4(103967) c5(103968, 103969, 103970, 103971, 103972, 103973) c6( ) c7(bt, aw); #c1(PHF23) c2(NP_077273) c3(PHD finger protein 23 isoform 1) c4(214862, 214863) c5(214864, 214865, 214866, 214867, 214868, 214869, 214870, 214871, 214872, 214873, 214874, 214875, 214876, 214877) c6( ) c7(bt, aw); #c1(PHF3) c2(NP_001277189) c3(PHD finger protein 3 isoform 3) c4( ) c5(109209) c6( ) c7(d); #c1(PHF3) c2(NP_055968) c3(PHD finger protein 3 isoform 1) c4(184780) c5(184781, 184782, 184783, 184784, 184785, 184786, 184787, 184788) c6( ) c7(d); #c1(PHF3) c2(XP_011533950) c3(PHD finger protein 3 isoform X3) c4( ) c5(330146, 330147) c6( ) c7(d); #c1(PHF5A) c2(NP_116147) c3(PHD finger-like domain-containing protein 5A) c4(225485) c5(225486, 225487, 225488, 225489, 225490, 225491) c6( ) c7(dB, Or, app, h); #c1(PHF6) c2(NP_115711) c3(PHD finger protein 6 isoform 2) c4(223680) c5(223681, 223682, 223683, 223684, 223685, 223686) c6( ) c7(T, ou, iZ, bsC, oA, fd, nf, nA, agk, bt, B, UE); #c1(PHF6) c2(NP_115834) c3(PHD finger protein 6 isoform 1) c4(224220, 224221, 224222, 224223) c5(224224, 224225, 224226, 224227, 224228, 224229, 224230, 224231, 224232, 224233, 224234, 224235, 224236, 224237, 224238, 224239, 224240, 224241, 224242, 224243, 224244, 224245, 224246, 224247, 224248, 224249, 224250, 224251) c6( ) c7(T, ou, iZ, bsC, oA, fd, nf, nA, agk, bt, B, UE); #c1(PHF8) c2(NP_001171825) c3(histone lysine demethylase PHF8 isoform 1) c4(71868, 71869, 71870, 71871, 71872) c5(71873, 71874, 71875, 71876, 71877, 71878, 71879, 71880, 71881, 71882, 71883, 71884) c6( ) c7(T, amJ, it, nf, amj, Nc, jj, nA, he, ae, ad, bN, bqN); #c1(PHF8) c2(NP_001171826) c3(histone lysine demethylase PHF8 isoform 3) c4(71885) c5(71886, 71887, 71888, 71889, 71890) c6( ) c7(T, amJ, it, nf, amj, Nc, j, nA, he, ae, ad, bN, bqN); #c1(PHF8) c2(NP_001171827) c3(histone lysine demethylase PHF8 isoform 4) c4(71891) c5(71892, 71893, 71894, 71895, 71896, 71897, 71898, 71899, 71900, 71901, 71902, 71903, 71904, 71905, 71906, 71907, 71908, 71909, 71910, 71911, 71912, 71913, 71914, 71915, 71916, 71917, 71918, 71919, 71920, 71921, 71922) c6( ) c7(T, amJ, it, nf, amj, Nc, j, nA, he, ae, ad, bN, bqN); #c1(PHF8) c2(NP_055922) c3(histone lysine demethylase PHF8 isoform 2) c4(184482) c5( ) c6( ) c7(T, amJ, it, nf, amj, Nc, j, nA, he, ae, ad, bN, bqN); #c1(PHF8) c2(XP_005262053) c3(histone lysine demethylase PHF8 isoform X1) c4(277734, 277735) c5( ) c6( ) c7(T, amJ, it, nf, amj, Nc, jj, nA, he, ae, ad, bN, bqN); #c1(PHF8) c2(XP_005262057) c3(histone lysine demethylase PHF8 isoform X3) c4(277736) c5(277737, 277738, 277739, 277740, 277741, 277742, 277743) c6( ) c7(T, amJ, it, nf, amj, Nc, jj, nA, he, ae, ad, bN, bqN); #c1(PHF8) c2(XP_006724648) c3(histone lysine demethylase PHF8 isoform X4) c4( ) c5(296162, 296163, 296164, 296165, 296166, 296167, 296168, 296169, 296170, 296171, 296172, 296173, 296174, 296175, 296176) c6( ) c7(T, amJ, it, nf, amj, Nc, jj, nA, he, ae, ad, bN, bqN); #c1(PHGDH) c2(XP_011539533) c3(D-3-phosphoglycerate dehydrogenase isoform X6) c4( ) c5(337731, 337732, 337733, 337734, 337735, 337736, 337737, 337738, 337739, 337740, 337741) c6( ) c7(bM, T, aTS, mC, ad, eA, eW, aT, vH, K, rz, cqw, Qe, Nr, iU, Jl, aW, Y, eH, eZ, po, lo, cqx, cqq, Ku, eF, t, jT, QK, f, ek, dR, Nq, bF, BU, Ew, Uu, c, dB, dc, m, qF, fL, eQ, WL, ahZ); #c1(PHIP) c2(NP_060404) c3(PH-interacting protein) c4(195821) c5(195822, 195823, 195824, 195825, 195826, 195827, 195828) c6( ) c7(Y, fN, eA, UF, bHF, yn); #c1(PHIP) c2(XP_011534220) c3(PH-interacting protein isoform X3) c4(330571) c5(330572, 330573, 330574, 330575, 330576) c6( ) c7(Y, fN, eA, UF, bHF, yn); #c1(PHKA1) c2(NP_001116142) c3(phosphorylase b kinase regulatory subunit alpha, skeletal muscle isoform isoform 2) c4(43647) c5(43648, 43649, 43650, 43651, 43652, 43653) c6( ) c7(cqy, acY, Bw, bq, gT, on, add); #c1(PHKA1) c2(NP_001165907) c3(phosphorylase b kinase regulatory subunit alpha, skeletal muscle isoform isoform 3) c4(67988) c5(67989, 67990, 67991, 67992, 67993, 67994) c6( ) c7(cqy, acY, Bw, bq, gT, on, add); #c1(PHKA1) c2(NP_002628) c3(phosphorylase b kinase regulatory subunit alpha, skeletal muscle isoform isoform 1) c4(130452, 130453) c5(130454, 130455, 130456, 130457, 130458, 130459, 130460, 130461, 130462, 130463, 130464, 130465) c6( ) c7(cqy, acY, Bw, bq, gT, on, add); #c1(PHKA1) c2(XP_006724724) c3(phosphorylase b kinase regulatory subunit alpha, skeletal muscle isoform isoform X1) c4(296270) c5(296271, 296272, 296273, 296274, 296275, 296276) c6( ) c7(cqy, acY, Bw, bq, gT, on, add); #c1(PHKA2) c2(NP_000283) c3(phosphorylase b kinase regulatory subunit alpha, liver isoform) c4(6171) c5(6172, 6173, 6174, 6175, 6176, 6177, 6178, 6179, 6180, 6181, 6182, 6183, 6184, 6185, 6186, 6187, 6188, 6189, 6190, 6191, 6192, 6193, 6194, 6195, 6196, 6197, 6198, 6199, 6200, 6201, 6202, 6203, 6204, 6205, 6206, 6207, 6208, 6209, 6210, 6211, 6212, 6213, 6214, 6215, 6216, 6217, 6218, 6219, 6220, 6221, 6222, 6223, 6224, 6225, 6226, 6227, 6228, 6229, 6230, 6231, 6232, 6233, 6234, 6235, 6236, 6237, 6238, 6239, 6240, 6241, 6242, 6243, 6244, 6245, 6246, 6247, 6248, 6249, 6250, 6251, 6252, 6253, 6254, 6255, 6256, 6257, 6258, 6259, 6260, 6261, 6262, 6263, 6264) c6(6265) c7(cqz, cqA, acY, Bw, ex, on, add); #c1(PHKB) c2(NP_000284) c3(phosphorylase b kinase regulatory subunit beta isoform a) c4(6266, 6267) c5(6268, 6269, 6270, 6271, 6272, 6273, 6274, 6275, 6276, 6277, 6278, 6279, 6280, 6281, 6282, 6283, 6284, 6285, 6286, 6287, 6288, 6289, 6290, 6291, 6292, 6293, 6294, 6295, 6296, 6297, 6298, 6299, 6300, 6301, 6302, 6303, 6304, 6305, 6306, 6307, 6308, 6309, 6310, 6311, 6312, 6313, 6314, 6315, 6316, 6317, 6318, 6319, 6320, 6321, 6322, 6323, 6324, 6325, 6326, 6327, 6328, 6329, 6330, 6331, 6332, 6333, 6334, 6335, 6336, 6337, 6338, 6339, 6340, 6341, 6342, 6343, 6344, 6345, 6346, 6347, 6348, 6349, 6350, 6351, 6352, 6353, 6354, 6355, 6356, 6357, 6358, 6359, 6360, 6361, 6362, 6363, 6364, 6365, 6366) c6(6367) c7(cqB, acY, Bw); #c1(PHKB) c2(XP_005256041) c3(phosphorylase b kinase regulatory subunit beta isoform X1) c4(273919) c5(273920, 273921, 273922, 273923, 273924, 273925, 273926, 273927, 273928, 273929) c6( ) c7(cqB, acY, Bw); #c1(PHKG1) c2(NP_001245388) c3(phosphorylase b kinase gamma catalytic chain, skeletal muscle/heart isoform isoform 1) c4(93428) c5(93429, 93430, 93431, 93432, 93433, 93434) c6( ) c7(Bw); #c1(PHKG1) c2(NP_001245389) c3(phosphorylase b kinase gamma catalytic chain, skeletal muscle/heart isoform isoform 3) c4(93435) c5( ) c6( ) c7(Bw); #c1(PHKG1) c2(NP_006204) c3(phosphorylase b kinase gamma catalytic chain, skeletal muscle/heart isoform isoform 2) c4(162820) c5(162821, 162822, 162823, 162824, 162825, 162826, 162827) c6( ) c7(Bw); #c1(PHKG1) c2(XP_005271829) c3(phosphorylase b kinase gamma catalytic chain, skeletal muscle/heart isoform isoform X1) c4( ) c5(283605, 283606, 283607, 283608, 283609) c6( ) c7(Bw); #c1(PHKG2) c2(NP_000285) c3(phosphorylase b kinase gamma catalytic chain, liver/testis isoform isoform 1) c4(6368) c5(6369, 6370, 6371, 6372, 6373, 6374, 6375, 6376, 6377, 6378, 6379, 6380, 6381, 6382, 6383, 6384, 6385, 6386, 6387, 6388, 6389, 6390, 6391, 6392, 6393, 6394, 6395, 6396, 6397, 6398, 6399, 6400, 6401, 6402, 6403, 6404, 6405, 6406, 6407, 6408, 6409, 6410, 6411, 6412, 6413, 6414, 6415, 6416, 6417, 6418, 6419, 6420, 6421, 6422, 6423, 6424, 6425, 6426, 6427, 6428, 6429, 6430, 6431, 6432, 6433, 6434, 6435, 6436, 6437, 6438, 6439, 6440, 6441, 6442, 6443, 6444, 6445, 6446, 6447, 6448, 6449, 6450, 6451, 6452, 6453, 6454, 6455, 6456, 6457, 6458, 6459, 6460, 6461, 6462, 6463, 6464, 6465) c6(6466) c7(cqC, gk, acY, al, Bw); #c1(PHKG2) c2(NP_00165903) c3 (phosphorylase b kinase gamma catalytic chain, liver/testis isoform isoform 2) c4(67974) c5(67975, 67976, 67977, 67978, 67979, 67980) c6( ) c7(cqC, gk, acY, al, Bw); #c1(PHLDA1) c2(NP_031376) c3(pleckstrin homology-like domain family A member 1) c4(172047, 172048) c5(172049, 172050, 172051, 172052, 172053, 172054, 172055, 172056, 172057, 172058, 172059, 172060, 172061, 172062, 172063, 172064, 172065, 172066, 172067, 172068, 172069) c6( ) c7(T, bE, aW, Y, fJ, ha, aT, bB, ey, er, cp); #c1(PHLDA2) c2(NP_003302) c3(pleckstrin homology-like domain family A member 2) c4(137267) c5(137268, 137269, 137270, 137271, 137272, 137273) c6( ) c7(T, d, eM, dB, eH, eF, cm, er, bN); #c1(PHLDA3) c2(NP_036528) c3 (pleckstrin homology-like domain family A member 3) c4(174239) c5(174240, 174241, 174242, 174243, 174244, 174245, 174246, 174247, 174248, 174249) c6( ) c7(aT); #c1(PHLDB1) c2(NP_001138231) c3(pleckstrin homology-like domain family B member 1 isoform b) c4(56081, 56082) c5(56083, 56084, 56085, 56086, 56087, 56088) c6( ) c7(bM, d, dB, mQ, eZ, aJu, eA); #c1(PHLDB1) c2(NP_055972) c3 (pleckstrin homology-like domain family B member 1 isoform a) c4(184813) c5(184814, 184815, 184816, 184817, 184818) c6( ) c7(bM, d, dB, mQ, eZ, aJu, eA); #c1(PHLDB1) c2(XP_005271526) c3(pleckstrin homology-like domain family B member 1 isoform X16) c4(283439) c5(283440, 283441, 283442, 283443, 283444, 283445) c6( ) c7(bM, d, dB, mQ, eZ, aJu, eA); #c1(PHLDB1) c2(XP_005271527) c3(pleckstrin homology-like domain family B member 1 isoform X17) c4( ) c5(283446) c6( ) c7(bM, d, dB, mQ, eZ, aJu, eA); #c1(PHLDB1) c2(XP_005271531) c3 (pleckstrin homology-like domain family B member 1 isoform X25) c4( ) c5(283447) c6( ) c7(bM, d, dB, mQ, eZ, aJu, eA); #c1(PHLDB1) c2(XP_006718859) c3(pleckstrin homology-like domain family B member 1 isoform X8) c4(292024) c5( ) c6( ) c7(bM, d, dB, ml, eZ, aJu, BA); #c1(PHLDB1) c2(XP_006718863) c3(pleckstrin homology-like domain family B member 1 isoform X13) c4(292025) c5(292026, 292027, 292028, 292029, 292030, 292031) c6( ) c7(bM, d, dB, mQ, eZ, aJu, eA); #c1(PHLDB1) c2(XP_006718865) c3(pleckstrin homology-like domain family B member 1 isoform X19) c4(292032) c5( ) c6( ) c7(bM, d, dB, mQ, eZ, aJu, eA); #c1(PHLDB1) c2(XP_011541016) c3(pleckstrin homology-like domain family B member 1 isoform X28) c4( ) c5(339540, 339541, 339542, 339543, 339544, 339545, 339546, 339547, 339548, 339549, 339550, 339551) c6( ) c7(bM, d, dB, mQ, eZ, aJu, eA); #c1(PHLDB1) c2(XP_011541017) c3(pleckstrin homology-like domain family B member 1 isoform X29) c4( ) c5(339552, 339553, 339554, 339555, 339556, 339557) c6( ) c7(bM, d, dB, mQ, eZ, aJu, eA); #c1(PHLPP1) c2(NP_919431) c3(PH domain leucine-rich repeat-containing protein phosphatase 1) c4(258607, 258608) c5(258609, 258610, 258611, 258612, 258613, 258614, 258615, 258616, 258617, 258618, 258619, 258620) c6( ) c7(bL, T, jh, rf, bB, v, q, ad, cF, aT, ae, eu, d, B, s, aW, Y, r, aq, g, as, bz, ac, eM, bE, cj, X); #c1(PHLPP2) c2(NP_001275932) c3(PH domain leucine-rich repeat-containing protein phosphatase 2 isoform 2) c4(107441, 107442) c5(107443, 107444, 107445, 107446, 107447, 107448, 107449, 107450, 107451, 107452, 107453, 107454, 107455, 107456, 107457, 107458, 107459) c6( ) c7(T, eM, aW, r, aT, q, eA, aue); #c1(PHLPP2) c2(NP_055835) c3(PH domain leucine-rich repeat-containing protein phosphatase 2 isoform 1) c4(183771, 183772, 183773, 183774) c5(183775, 183776, 183777, 183778, 183779, 183780, 183781, 183782, 183783, 183784, 183785, 183786, 183787, 183788, 183789, 183790, 183791, 183792, 183793, 183794, 183795, 183796, 183797) c6( ) c7(T, eM, aW, r, aT, q, eA, aue); #c1(PHOX2A) c2(NP_005160) c3(paired mesoderm homeobox protein 2A) c4(153520) c5(153521, 153522, 153523, 153524, 153525, 153526, 153527) c6( ) c7(bL, amd, ha, cz, nf, cm, cqD, bxw, zW, f); #c1(PHOX2B) c2(NP_003915) c3(paired mesoderm homeobox protein 2B) c4(142139) c5(142140, 142141, 142142, 142143, 142144, 142145) c6( ) c7(xN, cZ, T, ha, atS, n, fw, aLl, cqH, Tg, fX, nX, dd, an, ahE, cqG, li, A, ahF, fe, ahl, EZ, iV, cm, cqE, bdn, cql, amd, dB, cqF, Ji, xb); #c1(PHPT1) c2(NP_001129333) c3(14 kDa phosphohistidine phosphatase isoform 2) c4(51395) c5(51396, 51397, 51398, 51399, 51400, 51401, 51402, 51403, 51404, 51405) c6( ) c7(h, bz); #c1(PHPT1) c2(NP_054891) c3(14 kDa phosphohistidine phosphatase isoform 3) c4(178227) c5(178228, 178229, 178230, 178231, 178232, 178233) c6( ) c7(h, bz); #c1(PHRF1) c2(NP_001273510) c3(PHD and RlNG finger domain-containing protein 1 isoform 1) c4(105104) c5(105105, 105106, 105107, 105108, 105109, 105110) c6( ) c7(qw, T, aW, eZ, aT, fL, CQ, y, mC); #c1(PHRF1) c2(NP_065952) c3(PHD and RlNG finger domain-containing protein 1 isoform 2) c4(206471) c5(206472, 206473, 206474, 206475, 206476, 206477) c6( ) c7(qw, T, aW, eZ, aT, fL, CQ, y, mC); #c1(PHTF1) c2(XP_011538833) c3(putative homeodomain transcription factor 1 isoform X1) c4(336819, 336820, 336821, 336822) c5(336823, 336824, 336825, 336826, 336827, 336828, 336829, 336830, 336831, 336832, 336833, 336834, 336835, 336836, 336837, 336838, 336839, 336840, 336841, 336842) c6( ) c7(A); #c1(PHTF1) c2(XP_011538835) c3(putative homeodomain transcription factor 1 isoform X4) c4(336843, 336844, 336845, 336846) c5(336847, 336848, 336849, 336850, 336851, 336852, 336853, 336854, 336855, 336856, 336857, 336858) c6( ) c7(A); #c1(PHTF2) c2(NP_001120830) c3(putative homeodomain transcription factor 2 isoform 2) c4(44783, 44784) c5(44785, 44786, 44787, 44788, 44789, 44790, 44791, 44792, 44793, 44794, 44795, 44796) c6( ) c7(hH, fq, pN); #c1(PHTF2) c2(NP_001120831) c3 (putative homeodomain transcription factor 2 isoform 3) c4(44797) c5(44798, 44799, 44800, 44801, 44802, 44803) c6( ) c7(hH, fq, pN); #c1(PHTF2) c2(NP_001120832) c3(putative homeodomain transcription factor 2 isoform 4) c4(44804) c5(44805, 44806, 44807, 44808, 44809, 44810) c6( ) c7(hH, fq, pN); #c1(PHTF2) c2(XP_005250566) c3(putative homeodomain transcription factor 2 isoform X1) c4(270379, 270380, 270381) c5(270382, 270383, 270384, 270385, 270386, 270387, 270388, 270389, 270390, 270391, 270392, 270393, 270394, 270395, 270396, 270397, 270398, 270399, 270400, 270401, 270402, 270403) c6( ) c7(hH, fq, pN); #c1(PHYH) c2(NP_001032626) c3(phytanoyl-CoA dioxygenase, peroxisomal isoform b) c4( ) c5(31399, 31400, 31401, 31402, 31403) c6( ) c7(aBn, cqJ); #c1(PHYH) c2(NP_006205) c3(phytanoyl-CoA dioxygenase, peroxisomal isoform a precursor) c4(162828, 162829) c5(162830, 162831, 162832, 162833, 162834, 162835, 162836) c6( ) c7(aBn, cqJ); #c1(PHYHIP) c2(NP_001092805) c3 (phytanoyl-CoA hydroxylase-interacting protein) c4(39636, 39637) c5(39638, 39639, 39640, 39641, 39642, 39643, 39644, 39645, 39646, 39647, 39648, 39649, 39650, 39651, 39652, 39653) c6( ) c7(f); #c1(PHYKPL) c2(NP_699204) c3(5-phosphohydroxy-L-lysine phospho-lyase isoform 2) c4(245905) c5(245906, 245907, 245908, 245909, 245910, 245911) c6( ) c7(bMR, bMS); #c1(PI15) c2(NP_056970) c3(peptidase inhibitor 15 preproprotein) c4(188262) c5(188263, 188264, 188265, 188266, 188267, 188268, 188269, 188270, 188271, 188272, 188273, 188274, 188275, 188276) c6(188277, 188278, 188279, 188280) c7(cqK); #c1(PI16) c2(NP_001186088) c3 (peptidase inhibitor 16 precursor) c4(77092, 77093, 77094) c5(77095, 77096, 77097, 77098, 77099, 77100, 77101, 77102, 77103, 77104, 77105, 77106, 77107, 77108, 77109, 77110) c6(77111, 77112, 77113, 77114) c7(ae, ad); #c1(PI16) c2(XP_005248974) c3 (peptidase inhibitor 16 isoform X2) c4(268805) c5(268806, 268807, 268808, 268809, 268810, 268811, 268812, 268813) c6(268814, 268815) c7(ae, ad); #c1(PI3) c2(NP_002629) c3(elafin preproprotein) c4(130466, 130467, 130468, 130469) c5(130470, 130471, 130472, 130473, 130474, 130475) c6( ) c7(afr, sS, d, fR, aw, aT, cr, fF, at, Xs, wR, h, xM, K, wC, iV, aEW, eW, ao, aa); #c1(PI4K2A) c2(NP_060895) c3(phosphatidylinositol 4-kinase type 2-alpha) c4(198446, 198447) c5(198448, 198449, 198450, 198451, 198452, 198453, 198454, 198455, 198456, 198457, 198458, 198459) c6( ) c7(B, lF); #c1(PI4K2B) c2(NP_060793) c3(phosphatidylinositol 4-kinase type 2-beta) c4(197859, 197860) c5(197861, 197862, 197863, 197864, 197865, 197866, 197867, 197868, 197869, 197870, 197871, 197872) c6( ) c7(he, bK); #c1(PI4KA) c2(NP_477352) c3 (phosphatidylinositol 4-kinase alpha) c4(230759, 230760) c5(230761, 230762, 230763, 230764, 230765, 230766, 230767, 230768, 230769, 230770, 230771, 230772, 230773, 230774, 230775, 230776, 230777, 230778, 230779, 230780, 230781) c6( ) c7(agd, cqL, RX, xa, bK, he, B, vi); #c1(PI4KA) c2(XP_005261691) c3(phosphatidylinositol 4-kinase alpha isoform X1) c4(277593) c5( ) c6( ) c7(agd, cqL, RX, xa, bK, he, B, vi); #c1(PI4KA) c2(XP_011528530) c3(phosphatidylinositol 4-kinase alpha isoform X5) c4( ) c5(323671) c6( ) c7(agd, cqL, RX, xa, bK, he, B, vi); #c1(PI4 KB) c2(NP_001185703) c3(phosphatidylinositol 4-kinase beta isoform 2) c4( ) c5(76066, 76067, 76068, 76069, 76070, 76071, 76072) c6( ) c7(af); #c1(PI4 KB) c2(NP_001185704) c3(phosphatidylinositol 4-kinase beta isoform 3) c4( ) c5(76073, 76074, 76075, 76076, 76077, 76078, 76079, 76080, 76081, 76082) c6( ) c7(af); #c1(PI4 KB) c2(NP_002642) c3(phosphatidylinositol 4-kinase beta isoform 1) c4(130644, 130645, 130646, 130647) c5(130648, 130649, 130650, 130651, 130652, 130653, 130654, 130655, 130656, 130657, 130658, 130659, 130660, 130661, 130662, 130663) c6( ) c7(af); #c1(PI4 KB) c2(XP_005245321) c3(phosphatidylinositol 4-kinase beta isoform X4) c4(266451, 266452) c5(266453, 266454, 266455, 266456, 266457, 266458, 266459, 266460, 266461, 266462, 266463, 266464, 266465, 266466, 266467, 266468, 266469) c6( ) c7(af); #c1(PI4 KB) c2(XP_011507936) c3(phosphatidylinositol 4-kinase beta isoform X1) c4(298290) c5( ) c6( ) c7(af); #c1(PIAS1) c2(NP_057250) c3(E3 SUMO-protein ligase PIAS1) c4(189715, 189716) c5(189717, 189718, 189719, 189720, 189721, 189722, 189723, 189724, 189725, 189726, 189727, 189728, 189729) c6( ) c7(ck, T, eM, aW, aT, vw, ao, v, np, K, lQ, jj, Pk, V, ae, ad); #c1(PIAS2) c2(NP_004662) c3(E3 SUMO-protein ligase PIAS2 isoform beta) c4(148625, 148626) c5(148627, 148628, 148629, 148630, 148631, 148632, 148633, 148634, 148635, 148636, 148637, 148638, 148639, 148640, 148641, 148642, 148643) c6( ) c7(T, fM, rq, byn, Y, jh, cm, rs, K, m, t); #c1(PIAS2) c2(NP_775298) c3(E3 SUMO-protein ligase PIAS2 isoform alpha) c4(249925) c5(249926, 249927, 249928, 249929, 249930, 249931, 249932, 249933, 249934, 249935, 249936, 249937) c6( ) c7(T, fM, rq, byn, Y, jh, cm, rs, K, m, t); #c1(PIAS2) c2(XP_005258434) c3(E3 SUMO-protein ligase PIAS2 isoform X3) c4(275674) c5(275675, 275676, 275677, 275678, 275679) c6( ) c7(T, fM, rq, byn, Y, jh, cm, rs, K, m, t); #c1(PIAS2) c2(XP_006722636) c3(E3 SUMO-protein ligase PIAS2 isoform X7) c4(294788) c5( ) c6( ) c7(T, fM, rq, byn, Y, jh, cm, rs, K, m, t); #c1(PIAS4) c2(NP_056981) c3(E3 SUMO-protein ligase PIAS4) c4(188326) c5(188327, 188328, 188329, 188330, 188331, 188332, 188333) c6( ) c7(ck, T, aE, iU, bE, VB, cr, aA, bB, bw, aF, iJ, m, bC, t, ae, ad); #c1(PIBF1) c2(NP_006337) c3(progesterone-induced-blocking factor 1) c4(164146, 164147) c5(164148, 164149, 164150, 164151, 164152, 164153) c6( ) c7(aW, cqM, y, bA, aT); #c1(PIBF1) c2(XP_011533183) c3(progesterone-induced-blocking factor 1 isoform X1) c4(329310) c5( ) c6( ) c7(aW, cqM, y, bA, aT); #c1(PIBF1) c2(XP_011533187) c3(progesterone-induced-blocking factor 1 isoform X5) c4( ) c5(329311, 329312, 329313, 329314, 329315, 329316) c6( ) c7(aW, cqM, y, bA, aT); #c1(PIBF1) c2(XP_011533188) c3(progesterone-induced-blocking factor 1 isoform X6) c4( ) c5(329317, 329318, 329319, 329320, 329321, 329322) c6( ) c7(aW, cqM, y, bA, aT); #c1(PICALM) c2(NP_001008660) c3(phosphatidylinositol-binding clathrin assembly protein isoform 2) c4(23345) c5(23346, 23347, 23348, 23349, 23350, 23351) c6( ) c7(ck, T, dA, af, pc, cH, iT, an, cj, aT, rO, iY, gE, v, ji, hf, f, hF, bt, fK); #c1 (PICALM) c2(NP_001193875) c3(phosphatidylinositol-binding clathrin assembly protein isoform 3) c4(82897) c5(82898, 82899, 82900, 82901, 82902, 82903) c6( ) c7(ck, T, dA, af, pc, cH, iT, an, cj, aT, rO, iY, gE, v, ji, hf, f, hF, bt, fK); #c1(PICALM) c2(NP_001193876) c3(phosphatidylinositol-binding clathrin assembly protein isoform 4) c4(82904) c5(82905, 82906, 82907, 82908, 82909) c6( ) c7(ck, T, dA, af, pc, cH, iT, an, cj, aT, rO, iY, gE, v, ji, hf, f, hF, bt, fK); #c1(PICALM) c2(NP_009097) c3(phosphatidylinositol-binding clathrin assembly protein isoform 1) c4(170658) c5(170659, 170660, 170661, 170662, 170663, 170664, 170665, 170666, 170667, 170668) c6( ) c7(ck, T, dA, af, pc, cH, iT, an, cj, aT, rO, iY, gE, v, ji, hf, f, hF, bt, fK); #c1(PICALM) c2(XP_005274390) c3(phosphatidylinositol-binding clathrin assembly protein isoform X15) c4( ) c5(285066) c6( ) c7(ck, T, dA, af, pc, cH, iT, an, cj, aT, rO, iY, gE, v, ji, hf, f, hF, bt, fK); #c1(PICK1) c2(NP_001034673) c3(PRKCA-binding protein) c4(32048, 32049, 32050, 32051, 32052) c5(32053, 32054, 32055, 32056, 32057, 32058, 32059, 32060, 32061, 32062, 32063, 32064, 32065, 32066, 32067, 32068) c6( ) c7(gU, Kv, aMv, U, gl, sv, dG, cqN, xa); #c1(PICK1) c2(XP_011528819) c3(PRKCA-binding protein isoform X1) c4( ) c5(324209, 324210, 324211, 324212, 324213, 324214, 324215, 324216, 324217, 324218, 324219, 324220, 324221, 324222, 324223, 324224, 324225, 324226) c6( ) c7(gU, Kv, aMv, U, gl, sv, dG, cqN, xa); #c1(PID1) c2(NP_001094288) c3(PTB-containing, cubilin and LRP1-interacting protein isoform 2) c4(40660) c5(40661, 40662, 40663, 40664, 40665, 40666, 40667, 40668, 40669, 40670, 40671, 40672) c6( ) c7(jf, T, d, ac, dB, v, as, eA, aa); #c1(PID1) c2(NP_060403) c3(PTB-containing, cubilin and LRP1-interacting protein isoform 1) c4(195814) c5(195815, 195816, 195817, 195818, 195819, 195820) c6( ) c7(jf, T, d, ac, dB, v, as, eA, aa); #c1(PID1) c2(XP_011509686) c3(PTB-containing, cubilin and LRP1-interacting protein isoform X1) c4(300251, 300252) c5(300253, 300254, 300255, 300256, 300257, 300258, 300259, 300260, 300261, 300262) c6( ) c7(jf, T, d, ac, dB, v, as, eA, aa); #c1(PIDD1) c2(XP_011518511) c3(p53-induced death domain-containing protein 1 isoform X4) c4( ) c5(312615) c6( ) c7(gV, hG, Y, ff, fr, en); #c1(PIDD1) c2(XP_011518512) c3(p53-induced death domain-containing protein 1 isoform X1) c4(312616) c5(312617, 312618, 312619, 312620, 312621, 312622) c6( ) c7(gV, hG, Y, ff, fr, en); #c1(PIDD1) c2(XP_011518513) c3(p53-induced death domain-containing protein 1 isoform X2) c4(312623) c5(312624, 312625, 312626, 312627, 312628) c6( ) c7(gV, hG, Y, ff, fr, en); #c1(PIEZO1) c2(NP_001136336) c3(piezo-type mechanosensitive ion channel component 1) c4(54820, 54821, 54822) c5(54823, 54824, 54825, 54826, 54827, 54828, 54829, 54830, 54831, 54832, 54833, 54834, 54835, 54836, 54837, 54838, 54839, 54840) c6( ) c7(av, ft, Fd, aq, rz, B, y, L); #c1(PIEZO2) c2(NP_071351) c3 (piezo-type mechanosensitive ion channel component 2) c4(211141) c5(211142, 211143, 211144, 211145, 211146, 211147, 211148, 211149, 211150, 211151, 211152, 211153, 211154, 211155, 211156, 211157, 211158, 211159, 211160, 211161, 211162, 211163, 211164, 211165) c6( ) c7(aAf, eM, cqP, aw, zD, cqO, af); #c1(PIEZO2) c2(XP_011524026) c3(piezo-type mechanosensitive ion channel component 2 isoform X2) c4(318544) c5(318545, 318546, 318547, 318548, 318549, 318550) c6( ) c7(aAf, eM, cqP, aw, zD, cqO, af); #c1(PIEZO2) c2(XP_011524028) c3 (piezo-type mechanosensitive ion channel component 2 isoform X4) c4(318551, 318552, 318553) c5( ) c6( ) c7(aAf, eM, cqP, aw, zD, cqO, af); #c1(PIF1) c2(NP_001273426) c3(ATP-dependent DNA helicase PIF1 isoform b) c4(104872, 104873) c5(104874, 104875, 104876, 104877, 104878, 104879) c6( ) c7(T, aW, aT, tc, oH, K, ae, ad); #c1(PIF1) c2(XP_011520386) c3(ATP-dependent DNA helicase PIF1 isoform X1) c4(315238) c5(315239, 315240, 315241, 315242, 315243, 315244, 315245, 315246, 315247, 315248, 315249, 315250, 315251, 315252, 315253, 315254) c6( ) c7(T, aW, aT, tc, oH, K, ae, ad); #c1(PIFO) c2(NP_001287760) c3(protein pitchfork isoform 2) c4(112830) c5(112831, 112832, 112833, 112834, 112835, 112836) c6( ) c7(ckH); #c1(PIGA) c2(NP_002632) c3(phosphatidylinositol N-acetylglucosaminyltransferase subunit A isoform 1) c4(130490, 130491) c5(130492, 130493, 130494, 130495, 130496, 130497, 130498, 130499, 130500, 130501, 130502, 130503, 130504, 130505, 130506, 130507, 130508, 130509, 130510, 130511, 130512, 130513, 130514, 130515, 130516, 130517, 130518, 130519, 130520, 130521, 130522, 130523, 130524, 130525, 130526, 130527, 130528, 130529, 130530, 130531, 130532, 130533, 130534, 130535, 130536, 130537, 130538, 130539, 130540, 130541, 130542, 130543, 130544, 130545, 130546, 130547, 130548, 130549, 130550, 130551, 130552, 130553, 130554, 130555, 130556, 130557, 130558, 130559, 130560, 130561, 130562, 130563, 130564, 130565, 130566, 130567, 130568, 130569, 130570, 130571, 130572, 130573, 130574, 130575, 130576, 130577, 130578, 130579, 130580, 130581, 130582, 130583) c6( ) c7(ka, T, ha, fd, oY, nA, pe, aU, h, K, cqQ, pk, amA, aHo, bEj, aom, cqR, bw, dr, ck, aWZ, ki, HN, oh, dW, oX, xG, Wl, bt, fh, aHl); #c1(PIGA) c2(NP_065206) c3(phosphatidylinositol N-acetylglucosaminyltransferase subunit A isoform 3) c4(204582) c5(204583, 204584, 204585, 204586, 204587, 204588, 204589, 204590, 204591, 204592, 204593, 204594) c6( ) c7(ka, T, ha, fd, oY, nA, pe, aU, h, K, cqQ, pk, amA, aHo, bEj, aom, cqR, bw, dr, ck, aWZ, ki, HN, oh, dW, oX, xG, Wl, bt, fh, aHl); #c1(PIGF) c2(NP_002634) c3(phosphatidylinositol-glycan biosynthesis class F protein isoform 1) c4(130584) c5(130585, 130586, 130587, 130588, 130589, 130590, 130591) c6( ) c7(bL, gx, sY, vT, eZ, K, ci, m, C, uC, fh, Vs); #c1(PIGF) c2(NP_775097) c3(phosphatidylinositol-glycan biosynthesis class F protein isoform 2) c4(249495) c5(249496, 249497, 249498, 249499, 249500) c6( ) c7(bL, gx, sY, vT, eZ, K, ci, m, C, uC, fh, Vs); #c1(PIGF) c2(XP_005264426) c3(phosphatidylinositol-glycan biosynthesis class F protein isoform X2) c4(278933) c5(278934, 278935, 278936, 278937, 278938, 278939) c6( ) c7(bL, gx, sY, vT, eZ, K, ci, m, C, uC, fh, Vs); #c1(PIGG) c2(NP_001120650) c3(GPI ethanolamine phosphate transferase 2 isoform 1) c4(44365) c5(44366, 44367, 44368, 44369, 44370, 44371, 44372, 44373) c6( ) c7(dy, ac); #c1(PIGG) c2(NP_001275981) c3(GPI ethanolamine phosphate transferase 2 isoform 4) c4(107636) c5(107637, 107638, 107639, 107640, 107641, 107642) c6( ) c7(dy, ac); #c1(PIGG) c2(NP_001275984) c3(GPI ethanolamine phosphate transferase 2 isoform 6) c4(107643) c5(107644, 107645, 107646, 107647, 107648, 107649) c6( ) c7(dy, ac); #c1(PIGG) c2(NP_001275986) c3(GPI ethanolamine phosphate transferase 2 isoform 7) c4(107650) c5(107651, 107652, 107653, 107654, 107655, 107656, 107657, 107658) c6( ) c7(dy, ac); #c1(PIGG) c2(NP_060203) c3(GPI ethanolamine phosphate transferase 2 isoform 2) c4(194739, 194740) c5(194741, 194742, 194743, 194744, 194745, 194746) c6( ) c7(dy, ac); #c1(PIGG) c2(XP_005272346) c3(GPI ethanolamine phosphate transferase 2 isoform X9) c4( ) c5(284003) c6( ) c7(dy, ac); #c1(PIGG) c2(XP_011511794) c3(GPI ethanolamine phosphate transferase 2 isoform X5) c4( ) c5(302485, 302486, 302487, 302488, 302489) c6( ) c7(dy, ac); #c1(PIGL) c2(NP_004269) c3(N-acetylglucosaminyl-phosphatidylinositol de-N-acetylase precursor) c4(144735, 144736) c5(144737, 144738, 144739, 144740, 144741, 144742) c6( ) c7(pk, oh, fq, cqS); #c1(PIGL) c2(XP_011522382) c3(N-acetylglucosaminyl- phosphatidylinositol de-N-acetylase isoform X1) c4( ) c5(317064, 317065, 317066, 317067, 317068, 317069) c6( ) c7(pk, oh, fq, cqS); #c1(PIGM) c2(NP_660150) c3(GPI mannosyltransferase 1) c4(240409) c5(240410, 240411, 240412, 240413, 240414, 240415, 240416) c6( ) c7(aHo, ha, bEj); #c1(PIGN) c2(NP_789744) c3(GPI ethanolamine phosphate transferase 1) c4(252200) c5(252201, 252202, 252203, 252204, 252205, 252206, 252207, 252208, 252209, 252210) c6( ) c7(cqT, ha, cd, q, r); #c1(PIGO) c2(NP_001188413) c3(GPI ethanolamine phosphate transferase 3 isoform 2) c4(79180) c5(79181, 79182, 79183, 79184, 79185, 79186, 79187, 79188, 79189, 79190) c6( ) c7(Wl, lm, cqU, cql); #c1(PIGO) c2(NP_116023) c3(GPI ethanolamine phosphate transferase 3 isoform 1) c4(225239) c5(225240, 225241, 225242, 225243, 225244, 225245, 225246, 225247, 225248, 225249, 225250) c6( ) c7(Wl, lm, cqU, cql); #c1(PIGP) c2(NP_710148) c3(phosphatidylinositol N-acetylglucosaminyltransferase subunit P isoform 1) c4(246481, 246482, 246483, 246484) c5(246485, 246486, 246487, 246488, 246489, 246490, 246491, 246492, 246493, 246494) c6( ) c7(f, anj); #c1(PIGP) c2(NP_710149) c3(phosphatidylinositol N-acetylglucosaminyltransferase subunit P isoform 2) c4( ) c5(246495, 246496, 246497, 246498, 246499, 246500, 246501, 246502, 246503, 246504, 246505, 246506, 246507, 246508, 246509, 246510, 246511, 246512, 246513, 246514, 246515, 246516) c6( ) c7(f, anj); #c1(PIGQ) c2(NP_004195) c3(phosphatidylinositol N-acetylglucosaminyltransferase subunit @ isoform 2) c4(144241, 144242, 144243, 144244, 144245, 144246, 144247) c5(144248, 144249, 144250, 144251, 144252, 144253, 144254, 144255, 144256, 144257, 144258, 144259, 144260, 144261, 144262, 144263, 144264, 144265, 144266, 144267, 144268, 144269, 144270, 144271, 144272, 144273, 144274, 144275, 144276, 144277, 144278, 144279, 144280, 144281, 144282, 144283, 144284, 144285, 144286, 144287, 144288, 144289, 144290, 144291, 144292, 144293) c6( ) c7(cZ, ac, dd); #c1(PIGQ) c2(NP_683721) c3(phosphatidylinositol N-acetylglucosaminyltransferase subunit Q isoform 1) c4(242516) c5(242517, 242518, 242519, 242520, 242521, 242522, 242523, 242524) c6(242525, 242526) c7(cZ, ac, dd); #c1(PIGR) c2(XP_011507931) c3(polymeric immunoglobulin receptor isoform X1) c4(298261) c5(298262, 298263, 298264, 298265, 298266, 298267) c6( ) c7(oO, T, EM, fM, bB, n, bS, ct, ci, hX, cqV, aE, ja, ey, ji, B, ff, en, cl, Z, g, bz, eM, bE, hL, dy, hE, X); #c1(PIGT) c2(NP_001171657) c3(GPI transamidase component PIG-T isoform 2 precursor) c4(71168) c5(71169, 71170, 71171, 71172, 71173, 71174) c6( ) c7(cqW, hb, TJ, atf, fh, cqX); #c1(PIGT) c2(NP_001171658) c3(GPI transamidase component PIG-T isoform 3 precursor) c4(71175) c5(71176, 71177, 71178, 71179, 71180, 71181) c6( ) c7(cqW, hb, TJ, atf, fh, cqX); #c1(PIGT) c2(NP_001171659) c3(GPI transamidase component PIG-T isoform 4 precursor) c4(71182, 71183, 71184) c5(71185, 71186, 71187, 71188, 71189, 71190, 71191, 71192, 71193, 71194, 71195, 71196, 71197, 71198, 71199, 71200, 71201, 71202) c6( ) c7(cqW, hb, TJ, atf, fh, cqX); #c1(PIGT) c2(NP_057021) c3(GPI transamidase component PIG-T isoform 1 precursor) c4(188561, 188562) c5(188563, 188564, 188565, 188566, 188567, 188568, 188569) c6( ) c7(cqW, hb, TJ, atf, fh, cqX); #c1(PIGT) c2(XP_005260489) c3(GPI transamidase component PIG-T isoform X2) c4( ) c5(276567, 276568, 276569, 276570, 276571) c6( ) c7(cqW, hb, TJ, atf, fh, cqX); #c1(PIGU) c2(NP_536724) c3(phosphatidylinositol glycan anchor biosynthesis class U protein precursor) c4(231295, 231296, 231297, 231298) c5(231299, 231300, 231301, 231302, 231303, 231304, 231305, 231306, 231307, 231308, 231309, 231310, 231311, 231312, 231313, 231314, 231315, 231316) c6( ) c7(kT, T, d, Dy, kl, aW, Y, hk, eL, cj, aT, fd, dL, yO, ci, ik, bz, B, biS); #c1(PIGU) c2(XP_011526844) c3(phosphatidylinositol glycan anchor biosynthesis class U protein isoform X1) c4( ) c5(321192, 321193, 321194, 321195, 321196, 321197) c6( ) c7(kT, T, d, Dy, kl, aW, Y, hk, eL, cj, aT, fd, dL, yO, ci, iK, bz, B, biS); #c1(PIGW) c2(NP_848612) c3(phosphatidylinositol-glycan biosynthesis class W protein) c4(253977, 253978) c5(253979, 253980, 253981, 253982, 253983, 253984, 253985, 253986, 253987, 253988, 253989, 253990, 253991, 253992, 253993) c6( ) c7(x, By, bS, hp, m, k, ct, Z, t); #c1(PIGY) c2(NP_001036081) c3(phosphatidylinositol N-acetylglucosaminyltransferase subunit Y) c4(33949) c5(33950, 33951, 33952, 33953, 33954, 33955) c6( ) c7(pk, oh, sj); #c1(PIH1D1) c2(NP_060386) c3(PIH1 domain-containing protein 1) c4(195748) c5(195749, 195750, 195751, 195752, 195753, 195754) c6( ) c7(aW, aT); #c1(PIK3AP1) c2(NP_689522) c3(phosphoinositide 3-kinase adapter protein 1) c4(242984) c5(242985, 242986, 242987, 242988, 242989, 242990, 242991) c6( ) c7(ad); #c1(PIK3AP1) c2(XP_005269555) c3(phosphoinositide 3-kinase adapter protein 1 isoform X3) c4(282634, 282635) c5(282636, 282637, 282638, 282639, 282640, 282641, 282642, 282643, 282644, 282645, 282646) c6( ) c7(ad); #c1(PIK3C2A) c2(NP_002636) c3(phosphatidylinositol 4-phosphate 3-kinase C2 domain-containing subunit alpha) c4(130592, 130593) c5(130594, 130595, 130596, 130597, 130598, 130599, 130600, 130601, 130602, 130603, 130604, 130605, 130606, 130607, 130608, 130609, 130610, 130611, 130612, 130613, 130614, 130615, 130616, 130617) c6( ) c7(bL, no, auy, iT, ff, hL, en, ad, hm); #c1(PIK3C2A) c2(XP_011518488) c3(phosphatidylinositol 4-phosphate 3-kinase C2 domain-containing subunit alpha isoform X2) c4(312553, 312554) c5( ) c6( ) c7(bL, no, auy, iT, ff, hl, en, ad, hm); #c1(PIK3C2A) c2(XP_011518489) c3(phosphatidylinositol 4-phosphate 3-kinase C2 domain-containing subunit alpha isoform X4) c4(312555) c5(312556, 312557, 312558, 312559, 312560, 312561) c6( ) c7(bL, no, auy, iT, ff, hL, en, ad, hm); #c1(PIK3C2B) c2(NP_002637) c3 (phosphatidylinositol 4-phosphate 3-kinase C2 domain-containing subunit beta) c4(130618, 130619, 130620) c5(130621, 130622, 130623, 130624, 130625, 130626, 130627, 130628, 130629, 130630, 130631) c6( ) c7(T, ft, cz, pb, cm, eA, ad); #c1(PIK3C2B) c2(XP_005245315) c3(phosphatidylinositol 4-phosphate 3-kinase C2 domain-containing subunit beta isoform X2) c4(266441, 266442, 266443) c5(266444, 266445, 266446, 266447, 266448, 266449, 266450) c6( ) c7(T, ft, cz, pb, cm, eA, ad); #c1(PIK3C2B) c2(XP_011507935) c3(phosphatidylinositol 4-phosphate 3-kinase CZ domain-containing subunit beta isoform X3) c4( ) c5(298268, 298269, 298270, 298271, 298272, 298273, 298274, 298275, 298276, 298277, 298278, 298279, 298280, 298281, 298282, 298283, 298284, 298285, 298286, 298287, 298288, 298289) c6( ) c7(T, ft, cz, pb, cm, eA, ad); #c1(PIK3C2G) c2(NP_001275701) c3(phosphatidylinositol 4-phosphate 3-kinase C2 domain-containing subunit gamma isoform 1) c4(107/22) c5(107123, 107124, 107125, 107126, 107127, 107128) c6( ) c7(bL, dQ, aw); #c1(PIK3C2G) c2(NP_004561) c3(phosphatidylinositol 4-phosphate 3-kinase CZ domain-containing subunit gamma isoform 3) c4(147636) c5(147637, 147638, 147639, 147640, 147641, 147642, 147643, 147644, 147645, 147646) c6( ) c7(bL, dQ, aw); #c1(PIK3C3) c2(NP_002638) c3(phosphatidylinositol 3-kinase catalytic subunit type 3) c4(130632) c5(130633, 130634, 130635, 130636, 130637, 130638) c6( ) c7(nD, T, af, aW, oe, ht, aw, eZ, aT, ff, aj, bK, fL, m, en, Kw, fq, mC); #c1(PIK3CA) c2(NP_006209) c3(phosphatidylinositol 4,5-bisphosphate 3-kinase catalytic subunit alpha isoform) c4(162895, 162896, 162897) c5(162898, 162899, 162900, 162901, 162902, 162903, 162904, 162905, 162906, 162907, 162908, 162909, 162910, 162911, 162912, 162913, 162914, 162915) c6( ) c7(gh, aHq, T, bM, aC, fR, pf, YU, ea, if, al, fd, bB, lR, ci, auu, Qp, YD, ip, x, bv, gJ, aE, Xc, ata, Fo, pX, auB, ja, auF, K, hu, MO, pk, eL, Zm, cp, Qe, cre, cqY, xQ, eG, zQ, yo, Jl, aW, Ls, QB, hl, LZ, Qv, Qq, Qm, PQ, crf, Ku, g, as, eF, cm, aoS, GR, Qt, QA, aJg, qJ, iP, bw, eM, nB, iA, aJh, iT, mx, U, cj, Qw, k, m, in, ik, iu, dD, Or, aw, ht, NT, qy, n, aj, akt, fr, PY, qN, PR, cd, br, eW, biS, Bl, ft, jz, hw, cc, TK, Nn, gd, kH, h, jy, By, dL, ey, ox, eV, apG, Gw, aYF, dx, awV, crc, avc, MD, AQ, aJL, ge, aiL, crb, aq, kp, agF, pm, mo, Og, Ld, iN, Ye, i, aKA, W, cA, auG, dB, hk, Qu, bP, ayS, bo, hL, hj, hR, pg, Vy, qF, art, anx, Qz, it, B, AN, bL, fM, Dh, fG, WT, jx, eX, aA, v, yO, C, crd, Qo, AO, pd, lu, q, J, qH, zn, cqZ, er, eA, eJ, aHy, lg, hW, nv, Qg, BJ, qK, aT, cy, dz, Fx, ch, rz, ae, ne, af, amw, jf, qw, avg, cra, s, NE, oV, Y, PL, Or, nO, iU, r, aJl, lo, en, cl, LV, t, iK, pb, bN, ck, Ta, sS, nM, kP, Dy, ac, iM, au, WU, adR, mf, im, bt, xb, rA, KG, LS, VB, hS, hl, kh, id, ow, Dd, jh, aHv, bS, dG, eq, zU, brr, hF, Fc, iJ, ad, afb, hX, dP, av, iC, hd, ib, py, Db, yt, ds, NS, hD, bj, bkw, ji, lQ, fb, nJ, NM, Yi, PM, d, je, BO, qt, ff, eH, Of, eZ, btV, aeV, aF, btZ, bow, EZ, anW, iR, bz, dr, ek, Hp, LB, es, gk, crg, Ew, bF, bE, X, gZ, ei, Gu, i, y, aed, CW, jr, auy, Tc); #c1(PIK3CB) c2(NP_006210) c3(phosphatidylinositol 4,5-bisphosphate 3-kinase catalytic subunit beta isoform isoform 1) c4(162916, 162917, 162918, 162919, 162920) c5(162921, 162922, 162923, 162924, 162925, 162926, 162927, 162928, 162929, 162930, 162931, 162932, 162933, 162934, 162935, 162936, 162937, 162938, 162939, 162940, 162941, 162942, 162943, 162944, 162945, 162946, 162947, 162948, 162949, 162950, 162951, 162952, 162953, 162954) c6( ) c7(gh, T, bM, aC, pf, YU, ea, if, al, fd, bB, lR, ci, Qp, ip, x, bv, aE, ata, Fo, pX, ja, K, hu, MO, pk, eL, cp, Qe, cre, xQ, eG, yo, aW, Ls, hl, LZ, PQ, brr, Ku, g, as, eF, cm, aoS, GR, Qt, QA, iP, qJ, bw, eM, nB, iA, iT, mx, U, cj, Qw, k, m, ik, iu, dD, Or, aw, ht, qy, n, aj, akt, fr, PY, qN, PR, cd, br, eW, biS, ft, jz, hw, TK, gd, kH, h, eV, By, dL, ey, ox, apG, dx, crc, MD, AQ, ge, aiL, crb, aq, kp, Og, pm, Ld, Ye, iO, aKA, W, cA, bP, dB, Vy, bo, hL, hR, pg, qF, art, anx, it, B, bL, fM, Dh, fG, jx, eX, aA, v, yO, C, crd, Qo, lu, pd, q, J, qH, zn, er, eA, eJ, aHy, lg, hW, nv, Qg, BJ, aT, cy, dz, Fx, rz, ae, af, jf, Qr, nO, iU, oV, Y, r, lo, en, cL, LV, t, iK, pb, bN, ck, Ta, sS, nM, kP, ac, iM, au, adR, mf, bt, rA, kG, LS, VB, hS, hl, kh, id, ow, jh, aHv, bS, dG, eq, hF, iJ, ad, dP, av, iC, hd, ib, py, ds, hD, bj, bkw, ji, lQ, fb, nJ, NM, Yi, PM, d, je, ff, eH, Df, eZ, aeV, aF, iR, bz, dr, ek, Hp, LB, es, gk, crg, Ew, bE, X, gZ, ei, y, CW, jr, Tc); #c1(PIK3CB) c2(XP_011511198) c3(phosphatidylinositol 4,5-bisphosphate 3-kinase catalytic subunit beta isoform isoform X2) c4(301666) c5(301667, 301668, 301669, 301670, 301671, 301672) c6( ) c7(gh, T, bM, aC, pf, YU, ea, if, al, fd, bB, lR, ci, Qp, ip, x, bv, aE, ata, Fo, pX, ja, K, hu, MO, pk, eL, cp, Qe, cre, xQ, eG, yo, aW, Ls, hl, LZ, PQ, brr, Ku, g, as, eF, cm, aoS, GR, Qt, QA, iP, qJ, bw, eM, nB, iA, iT, mx, U, cj, Qw, k, m, ik, iu, dD, Or, aw, ht, qy, n, aj, aKt, fr, PY, qN, PR, cd, br, eW, bis, ft, jz, hw, TK, gd, kH, h, eV, By, dL, ey, ox, apG, dx, crc, MD, AQ, ge, aiL, crb, aq, kp, Og, pm, Ld, Ye, iO, aKA, W, cA, bP, dB, Vy, bo, hL, hR, pg, qF, art, anx, it, B, bL, fM, Dh, fG, jx, eX, aA, v, yO, C, crd, Qo, lu, pd, q, J, qH, zn, er, eA, eJ, aHy, lg, hW, nv, Qg, BJ, aT, cy, dz, Fx, rz, ae, af, jf, Qr, nO, iU, oV, Y, r, lo, en, cl, LV, t, ik, pb, bN, ck, Ta, sS, nM, kP, ac, iM, au, adR, mf, bt, rA, kG, LS, VB, hS, hl, kh, id, ow, jh, aHv, bS, dG, eq, hF, iJ, ad, dP, av, iC, hd, ib, py, ds, hD, bj, bkw, ji, lQ, fb, nJ, NM, Yi, PM, d, je, ff, eH, Of, eZ, aeV, aF, iR, bz, dr, ek, Hp, LB, es, gk, crg, Ew, bE, X, gZ, ei, y, CW, jr, Tc); #c1(PIK3CD) c2(XP_011539885) c3(phosphatidylinositol 4,5-bisphosphate 3-kinase catalytic subunit delta isoform isoform X5) c4(338155) c5(338156, 338157, 338158, 338159, 338160, 338161) c6( ) c7(gh, T, bM, aC, YU, ea, if, aT, fd, bB, lR, ci, Qp, ip, x, bv, aE, ata, Fo, pX, ja, K, hu, MO, pk, eL, cp, Qe, cre, xQ, eG, yo, aW, Ls, hl, PQ, brr, Ku, g, as, eF, cm, aoS, GR, Qt, QA, iP, qJ, bw, eM, nB, iA, iT, mx, U, cj, Qw, k, m, ik, iu, dD, Dr, aw, ht, qy, n, aj, aKt, fr, PY, qN, PR, cd, br, eW, bis, Oe, ft, jz, hw, TK, gd, kH, h, eV, By, dL, ey, ox, apG, dx, crc, MD, zb, AQ, ge, aiL, crb, aq, kp, Og, pm, Ld, Ye, iO, aKA, W, cA, bP, dB, Vy, bo, hL, hR, at, pg, qF, art, anx, it, B, bL, fM, dA, Dh, fG, jx, eX, aA, v, yO, C, crd, Qo, lu, pd, q, J, qH, zn, er, eA, eJ, aHy, lg, hW, nv, Qg, BJ, aT, cy, dz, Fx, rz, ae, af, jf, Qr, nO, iU, oV, Y, r, lo, en, cl, LV, t, iK, pb, bN, ck, cT, sS, Ta, kP, ac, nM, iM, au, adR, mf, bt, rA, kG, LS, VB, hS, hl, kh, id, ow, jh, aHv, dG, eq, hF, iJ, ad, dP, av, iC, hd, ib, py, ds, hD, bj, bkw, ji, lQ, fb, nJ, NM, Yi, PM, d, je, cgq, ff, eH, Of, cr, eZ, dN, aeV, aF, iR, bz, dr, ek, Hp, LB, es, gk, crg, Ew, bE, X, gZ, ei, y, CW, jr, Tc); #c1(PIK3CG) c2(NP_001269355) c3(phosphatidylinositol 4,5-bisphosphate 3-kinase catalytic subunit gamma isoform) c4(101255) c5(101256, 101257, 101258, 101259, 101260, 101261, 101262, 101263, 101264, 101265, 101266, 101267, 101268, 101269) c6( ) c7(oO, gh, T, bM, aC, fR, YU, ea, if, e, al, fd, bB, lR, ci, Qp, ajC, ip, x, bv, xE, aE, ata, Fo, pX, ja, K, qM, hu, MO, pk, eL, cp, Qe, cre, xQ, eG, yo, aW, Ls, hl, PQ, brr, Ku, g, hJ, akj, as, aoS, GR, eF, Qt, cm, qJ, QA, iP, eM, nB, iA, iT, mx, U, cj, Qw, k, m, ik, iu, dD, Dr, aw, ht, qy, n, aj, aKt, fr, PY, qN, PR, cd, br, ai, biS, eW, ft, jz, hw, TK, gd, kH, h, eV, By, dL, ey, ox, apG, dx, crc, MD, AQ, ge, aiJ, crb, aq, kp, Og, pm, Ld, Ye, iO, aKA, W, cA, bP, dB, Vy, bo, hL, hR, pg, qF, art, anx, it, B, bL, fM, Dh, fG, jx, eX, aA, cz, v, j, yD, C, crd, bX, Qo, pd, lu, q, J, qH, zn, er, eA, eJ, mj, aHy, lg, hW, nv, Qg, BJ, aT, cy, dz, Fx, rz, ae, af, jf, Qr, nO, iU, oV, Y, r, bw, lo, en, cl, LV, t, iK, pb, bN, ck, Ta, sS, nM, kP, ac, iM, au, adR, mf, afW, fD, bt, rA, kG, LS, VB, hS, Kd, hl, kh, id, ow, jh, aHv, dG, eq, hF, iJ, ad, dP, av, iC, hd, ib, py, ds, hD, bj, bkw, ji, lQ, fb, nJ, NM, Yi, PM, d, je, ff, eH, Of, cr, eZ, dN, aeV, aF, iR, bz, dr, ek, Hp, LB, es, gk, crg, Ew, bE, X, gZ, ei, y, CW, jr, Tc); #c1(PIK3CG) c2(XP_011514619) c3(phosphatidylinositol 4,5-bisphosphate 3-kinase catalytic subunit gamma isoform isoform X3) c4( ) c5(307308) c6( ) c7(oO, gh, T, bM, aC, fR, YU, ea, if, e, aT, fd, bB, lR, ci, Qp, ajC, ip, x, bv, xE, aE, ata, Fo, pX, ja, K, qM, hu, MO, pk, eL, cp, Qe, cre, xQ, eG, yo, aW, Ls, hl, PQ, brr, Ku, g, hJ, aKj, as, aoS, GR, eF, Qt, cm, qJ, QA, iP, eM, nB, iA, iT, mx, U, cj, Qw, k, m, ik, iu, dD, Or, aw, ht, qy, n, aj, akt, fr, PY, qN, PR, cd, br, ai, biS, eW, ft, jz, hw, TK, gd, kH, h, eV, By, dL, ey, ox, apG, dx, crc, MD, AQ, ge, aiL, crb, aq, kp, Og, pm, Ld, Ye, iO, aKA, W, cA, bP, dB, Vy, bo, hL, hR, pg, qF, art, anx, it, B, bL, fM, Dh, fG, jx, eX, aA, cz, v, j, yO, C, crd, bX, Qo, pd, lu, q, J, qH, zn, er, eA, eJ, mj, aHy, lg, hW, nv, Qg, BJ, aT, cy, dz, Fx, rz, ae, af, jf, Qr, nO, iU, oV, Y, r, bw, lo, en, cl, LV, t, ik, pb, bN, ck, Ta, sS, nM, kP, ac, iM, au, adR, mf, afW, fD, bt, rA, KG, LS, VB, hS, Kd, hl, kh, id, ow, jh, aHv, dG, eq, hF, iJ, ad, dP, av, iC, hd, ib, py, ds, hD, bj, bkw, ji, lQ, fb, nJ, NM, Yi, PM, d, je, ff, eH, Of, cr, eZ, dN, aeV, aF, iR, bz, dr, ek, Hp, LB, es, gk, crg, Ew, bE, X, gZ, ei, y, CW, jr, Tc); #c1(PIK3IP1) c2(NP_001129383) c3(phosphoinositide-3-kinase-interacting protein 1 isoform 2 precursor) c4(51406) c5(51407, 51408, 51409, 51410, 51411) c6( ) c7(ft); #c1(PIK3IP1) c2(NP_443112) c3(phosphoinositide-3-kinase-interacting protein 1 isoform 1 precursor) c4(229469, 229470, 229471, 229472) c5(229473, 229474, 229475, 229476, 229477, 229478, 229479, 229480, 229481, 229482, 229483, 229484, 229485, 229486, 229487, 229488, 229489, 229490, 229491, 229492, 229493, 229494, 229495, 229496, 229497) c6( ) c7(ft); #c1(PIK3R1) c2(NP_001229395) c3(phosphatidylinositol 3-kinase regulatory subunit alpha isoform 4) c4( ) c5(84353, 84354, 84355, 84356, 84357, 84358, 84359, 84360, 84361, 84362) c6( ) c7(bL, T, aC, id, eR, aZ, uG, ex, cz, bB, Gd, aj, dG, v, ci, fd, zP, eA, J, ad, oT, anA, LL, ata, aT, h, lG, cy, dL, eV, anz, ji, bq, pk, ae, af, eL, kl, zQ, d, B, Jl, aW, aq, kp, crh, cl, t, bz, GR, fq, asE, Ye, ck, ac, cA, eM, bE, hl, uz, ei, X); #c1(PIK3R1) c2(NP_852556) c3(phosphatidylinositol 3-kinase regulatory subunit alpha isoform 2) c4(255040) c5(255041, 255042, 255043, 255044, 255045, 255046) c6( ) c7(bL, T, aC, id, eR, aZ, uG, eX, cz, bB, Gd, aj, dG, v, ci, fd, zP, eA, J, ad, oT, anA, LL, ata, aT, h, lG, cy, dL, eV, anz, ji, bq, pk, ae, af, eL, kl, zQ, d, B, Jl, aW, aq, kp, crh, cl, t, bz, GR, fq, asE, Ye, ck, ac, cA, eM, bE, hL, uz, ei, X); #c1(PIK3R1) c2(NP_852664) c3(phosphatidylinositol 3-kinase regulatory subunit alpha isoform 1) c4(255080, 255081) c5(255082, 255083, 255084, 255085, 255086, 255087, 255088, 255089, 255090, 255091, 255092, 255093, 255094, 255095, 255096, 255097, 255098, 255099, 255100, 255101) c6( ) c7(bL, T, aC, id, eR, aZ, uG, eX, cz, bB, Gd, aj, dG, v, ci, fd, zP, eA, J, ad, oT, anA, LL, ata, aT, h, lG, cy, dL, eV, anz, ji, bq, pk, ae, af, eL, kl, zQ, d, B, Jl, aW, aq, kp, crh, cl, t, bz, GR, fq, asE, Ye, ck, ac, cA, eM, bE, hL, uz, ei, X); #c1(PIK3R1) c2(NP_852665) c3(phosphatidylinositol 3-kinase regulatory subunit alpha isoform 3) c4(255102) c5(255103, 255104, 255105, 255106, 255107, 255108, 255109, 255110) c6( ) c7(bL, T, aC, id, eR, aZ, uG, eX, cz, bB, Gd, aj, dG, v, ci, fd, zP, eA, J, ad, oT, anA, LL, ata, aT, h, lG, cy, dL, eV, anz, ji, bq, pk, ae, af, eL, kl, zQ, d, B, Jl, aW, aq, kp, crh, cl, t, bz, GR, fq, asE, Ye, ck, ac, cA, eM, bE, hL, uz, ei, X); #c1(PIK3R1) c2(XP_011541795) c3(phosphatidylinositol 3-kinase regulatory subunit alpha isoform X2) c4(340389, 340390) c5( ) c6( ) c7(bL, T, aC, id, eR, aZ, uG, eX, cz, bB, Gd, aj, dG, v, ci, fd, zP, eA, J, ad, oT, anA, LL, ata, aT, h, lG, cy, dL, eV, anz, ji, bq, pk, ae, af, eL, kl, zQ, d, B, Jl, aW, aq, kp, crh, cl, t, bz, GR, fq, asE, Ye, ck, ac, cA, eM, bE, hL, uz, ei, X); #c1(PIK3R2) c2(NP_005018) c3(phosphatidylinositol 3-kinase regulatory subunit beta) c4(152211) c5(152212, 152213, 152214, 152215, 152216, 152217) c6( ) c7(T, PM, oT, cA, eM, bE, aW, aZ, hL, aT, fd, aq, m, ei, cl, eA, t, B); #c1(PIK3R3) c2(NP_001290356) c3(phosphatidylinositol 3-kinase regulatory subunit gamma isoform 2) c4(115285) c5(115286, 115287, 115288, 115289, 115290, 115291) c6( ) c7(ck, T, y, F, xE, bP, A, dV, h, cy, uQ, ek, H, bt, bz, eA, amW, ad, L); #c1(PIK3R3) c2(NP_001290357) c3(phosphatidylinositol 3-kinase regulatory subunit gamma isoform 3) c4( ) c5(115292, 115293, 115294) c6( ) c7(ck, T, y, F, xE, bP, A, dV, h, cy, uQ, ek, H, bt, bz, eA, amW, ad, L); #c1(PIK3R3) c2(NP_001290358) c3(phosphatidylinositol 3-kinase regulatory subunit gamma isoform 4) c4(115295, 115296) c5(115297, 115298, 115299, 115300, 115301, 115302, 115303, 115304, 115305, 115306, 115307, 115308, 115309, 115310, 115311, 115312, 115313, 115314, 115315, 115316, 115317, 115318) c6( ) c7(ck, T, y, F, xE, bP, A, dV, h, cy, uQ, ek, H, bt, bz, eA, amW, ad, L); #c1(PIK3R4) c2(NP_055417) c3 (phosphoinositide 3-kinase regulatory subunit 4) c4(181029) c5(181030, 181031, 181032, 181033, 181034, 181035) c6( ) c7(XC, aiW, Lq, pl, m, ps); #c1(PIK3R5) c2(NP_055123) c3(phosphoinositide 3-kinase regulatory subunit 5 isoform 1) c4(179087) c5(179088, 179089, 179090, 179091, 179092, 179093, 179094, 179095, 179096, 179097) c6( ) c7(T, cri, KA, h, ci, KF); #c1(PIKFYVE) c2(NP_001171471) c3(1-phosphatidylinositol 3-phosphate 5-kinase isoform 4) c4( ) c5(70227, 70228, 70229, 70230, 70231, 70232) c6( ) c7(btl, T, id, W); #c1(PIKFYVE) c2(NP_055855) c3(1-phosphatidylinositol 3-phosphate 5-kinase isoform 2) c4(183934, 183935) c5(183936, 183937, 183938, 183939, 183940, 183941) c6( ) c7(btQ, T, id, W); #c1(PIKFYVE) c2(NP_689884) c3(1-phosphatidylinositol 3-phosphate 5-kinase isoform 3) c4(244246) c5(244247, 244248, 244249, 244250, 244251, 244252) c6( ) c7(btQ, T, id, W); #c1(PIKFYVE) c2(XP_011509086) c3(1-phosphatidylinositol 3-phosphate 5-kinase isoform X6) c4(299678) c5(299679, 299680, 299681, 299682, 299683, 299684) c6( ) c7(btQ, T, id, W); #c1(PILRA) c2(NP_038467) c3(paired immunoglobulin-like type 2 receptor alpha isoform 1 precursor) c4(176570, 176571) c5(176572, 176573, 176574, 176575, 176576, 176577) c6( ) c7(W, fR); #c1(PILRA) c2(NP_840056) c3(paired immunoglobulin-like type 2 receptor alpha isoform 2 precursor) c4(253593, 253594) c5(253595, 253596, 253597, 253598, 253599, 253600, 253601, 253602, 253603, 253604, 253605, 253606) c6( ) c7(W, FR); #c1(PILRA) c2(NP_840057) c3(paired immunoglobulin-like type 2 receptor alpha isoform 3 precursor) c4(253607) c5(253608, 253609, 253610, 253611, 253612, 253613) c6( ) c7(W, fR); #c1(PIM1) c2(NP_001230115) c3(serine/threonine-protein kinase pim-1 isoform 1) c4(85950) c5(85951) c6( ) c7(lD, T, am, fM, eX, jh, aA, fd, n, aj, ci, sl, cd, J, ad, x, av, cc, aE, bQV, kH, aT, cy, h, dL, eV, ae, N, ly, crj, xX, d, bZZ, aW, fJ, ff, hl, kp, aF, uF, en, hf, g, ik, bz, LS, lx, Cv, ck, eM, bE, cj, at, pg, k, bt, it, jr); #c1(PIM1) c2(NP_002639) c3(serine/threonine-protein kinase pim-1 isoform 2) c4( ) c5(130639, 130640, 130641, 130642, 130643) c6( ) c7(lD, T, am, fM, eX, jh, aA, fd, n, aj, ci, sl, cd, J, ad, x, av, cc, aE, bQV, KH, aT, cy, h, dL, eV, ae, N, ly, crj, xx, d, bZZ, aW, fJ, ff, hl, kp, af, uF, en, hf, g, iK, bz, LS, lx, Cv, ck, eM, bE, cj, at, pg, k, bt, it, jr); #c1(PIM2) c2(NP_006866) c3(serine/threonine-protein kinase pim-2) c4(168724, 168725) c5(168726, 168727, 168728, 168729, 168730, 168731, 168732, 168733, 168734, 168735, 168736, 168737) c6( ) c7(ck, ae, T, gk, fM, al, jh, cj, fd, n, jj, hF, bt, KS, B, ad); #c1(PIM3) c2(NP_001001852) c3(serine/threonine-protein kinase pim-3) c4(19041) c5(19042, 19043, 19044, 19045, 19046, 19047) c6( ) c7(ck, T, aC, d, eJ, s, aE, bE, cj, bB, aq, at, kp, g, mF, bt, B); #c1(PIN1) c2(NP_006212) c3(peptidyl-prolyl cis-trans isomerase NIMA-interacting 1) c4(162955, 162956, 162957) c5(162958, 162959, 162960, 162961, 162962, 162963, 162964, 162965, 162966, 162967, 162968, 162969, 162970, 162971, 162972, 162973, 162974, 162975) c6( ) c7(bM, T, AS, Ol, fG, if, cpJ, jh, n, hy, v, fr, ci, fu, qN, cd, ad, eA, hX, Hx, py, MR, WY, sO, qK, aT, h, dL, K, jj, an, ae, af, qw, d, aW, oV, vj, fc, Du, ff, akB, aaE, en, cm, iK, bz, ck, es, bb, c, nB, eM, iA, U, at, mf, np, cP, fD, y, bt, it, B); #c1(PIN4) c2(NP_001164218) c3(peptidyl-prolyl cis-trans isomerase NIMA-interacting 4 isoform 2) c4(66815) c5(66816, 66817, 66818, 66819, 66820, 66821) c6( ) c7(mf); #c1(PIN4) c2(NP_006214) c3(peptidyl-prolyl cis-trans isomerase NIMA-interacting 4 isoform 1) c4(162976) c5(162977, 162978, 162979, 162980, 162981, 162982) c6( ) c7(mf); #c1(PINX1) c2(NP_001271285) c3(PIN2/TERF1-interacting telomerase inhibitor 1 isoform 2) c4(103834, 103835) c5(103836, 103837, 103838, 103839, 103840, 103841, 103842, 103843, 103844, 103845, 103846, 103847) c6( ) c7(XX, iD, T, jf, id, aW, ff, eH, jh, aA, aT, aq, aF, ci, en, eF, B, hX); #c1(PINX1) c2(NP_060354) c3(PIN2/TERF1-interacting telomerase inhibitor 1 isoform 1) c4(195564) c5(195565, 195566, 195567, 195568, 195569, 195570) c6( ) c7(XX, iO, T, jf, id, aW, ff, eH, jh, aA, aT, aq, aF, ci, en, eF, B, hX); #c1(PIP) c2(NP_002643) c3 (prolactin-inducible protein precursor) c4(130664) c5(130665, 130666, 130667, 130668, 130669, 130670) c6( ) c7(T, KW, bB, Fb, zF, ci, nA, fr, atN, ai, ad, aT, rx, DO, K, aDZ, ae, crk, avg, aW, Ue, zD, Pp, g, atM, dB, bE, Pl, zB); #c1(PIP4K2A) c2(NP_005019) c3(phosphatidylinositol 5-phosphate 4-kinase type-2 alpha) c4(152218, 152219, 152220, 152221, 152222, 152223) c5(152224, 152225, 152226, 152227, 152228, 152229, 152230, 152231, 152232, 152233, 152234, 152235) c6( ) c7(x, mU, ac, mW, Y, jb, mZ, mS, sv, mY, he, k, bK, mV, mT, ad, mX); #c1(PIP4K2A) c2(XP_011517797) c3(phosphatidylinositol 5-phosphate 4-kinase type-2 alpha isoform X2) c4( ) c5(311701, 311702, 311703, 311704, 31/705, 31/706, 311707) c6( ) c7(x, mU, ac, mW, Y, jb, mZ, mS, sv, mY, he, k, bK, mV, mT, ad, mX); #c1(PIP4KZA) c2(XP_011517798) c3 (phosphatidylinositol 5-phosphate 4-kinase type-2 alpha isoform X4) c4( ) c5(311708, 311709, 311710, 311711, 311712, 311713, 311714, 311715, 311716, 311717, 311718) c6( ) c7(x, mU, ac, mW, Y, jb, mZ, mS, sv, mY, he, k, bK, mV, mT, ad, mX); #c1(PIP4KZA) c2(XP_011517799) c3(phosphatidylinositol 5-phosphate 4-kinase type-2 alpha isoform X5) c4( ) c5(311719, 311720, 311721, 311722, 311723, 311724) c6( ) c7(x, mU, ac, mW, Y, jb, mZ, mS, sv, mY, he, k, bK, mV, mT, ad, mX); #c1(PIP4K2B) c2(NP_003550) c3(phosphatidylinositol 5-phosphate 4-kinase type-2 beta) c4(139023, 139024) c5(139025, 139026, 139027, 139028, 139029, 139030, 139031, 139032, 139033, 139034, 139035, 139036) c6( ) c7(af, aT); #c1(PIP4K2C) c2(NP_001139731) c3(phosphatidylinositol 5-phosphate 4-kinase type-2 gamma isoform b) c4(59775) c5(59776, 59777, 59778, 59779, 59780, 59781) c6( ) c7(y, A); #c1(PIP4K2C) c2(NP_001139732) c3(phosphatidylinositol 5-phosphate 4-kinase type-2 gamma isoform c) c4(59782) c5(59783, 59784, 59785, 59786, 59787, 59788) c6( ) c7(y, A); #c1(PIP4K2C) c2(NP_079055) c3 (phosphatidylinositol 5-phosphate 4-kinase type-2 gamma isoform a) c4(216770, 216771) c5(216772, 216773, 216774, 216775, 216776, 216777, 216778, 216779, 216780, 216781, 216782, 216783, 216784, 216785, 216786, 216787) c6( ) c7(y, A); #c1(PIPSK1A) c2(NP_001129108) c3(phosphatidylinositol 4-phosphate 5-kinase type-1 alpha isoform 3) c4(50778) c5(50779, 50780, 50781, 50782, 50783, 50784) c6( ) c7(aW, aT); #c1(PIPSK1A) c2(NP_001129109) c3(phosphatidylinositol 4-phosphate 5-kinase type-1 alpha isoform 4) c4(50785) c5(50786, 50787, 50788, 50789, 50790, 50791) c6( ) c7(aW, aT); #c1(PIPSK1A) c2(NP_00129110) c3(phosphatidylinositol 4-phosphate 5-kinase type-1 alpha isoform 1) c4(50792) c5(50793, 50794, 50795, 50796, 50797, 50798) c6( ) c7(aW, aT); #c1(PIPSK1A) c2(NP_003548) c3(phosphatidylinositol 4-phosphate 5-kinase type-1 alpha isoform 2) c4(139016) c5(139017, 139018, 139019, 139020, 139021, 139022) c6( ) c7(aW, aT); #c1(PIPSK1A) c2(XP_005245588) c3(phosphatidylinositol 4-phosphate 5-kinase type-1 alpha isoform X14) c4(266760) c5(266761, 266762, 266763, 266764, 266765, 266766) c6( ) c7(aW, aT); #c1(PIPSK1A) c2(XP_011508349) c3(phosphatidylinositol 4-phosphate 5-kinase type-1 alpha isoform X17) c4(299038, 299039) c5(299040, 299041, 299042, 299043, 299044, 299045, 299046, 299047, 299048, 299049, 299050, 299051) c6( ) c7(aW, aT); #c1(PIPSK1B) c2(XP_006717364) c3(phosphatidylinositol 4-phosphate 5-kinase type-1 beta isoform X2) c4(290979, 290980) c5(290981, 290982, 290983, 290984, 290985, 290986, 290987, 290988, 290989, 290990, 290991, 290992, 290993, 290994, 290995, 290996, 290997, 290998, 290999, 291000, 291001, 291002, 291003, 291004, 291005, 291006, 291007, 291008, 291009, 291010) c6( ) c7(bM, eR, dy, aE); #c1(PIPSK1B) c2(XP_011517387) c3 (phosphatidylinositol 4-phosphate 5-kinase type-1 beta isoform X1) c4(311100, 311101, 311102, 311103) c5(311104, 311105, 311106, 311107, 311108, 311109, 311110, 311111, 311112, 311113) c6( ) c7(bM, eR, dy, aE); #c1(PIPSK1C) c2(NP_001182662) c3 (phosphatidylinositol 4-phosphate 5-kinase type-l gamma isoform 1) c4(75141) c5(75142, 75143, 75144, 75145, 75146) c6( ) c7(crl); #c1(PIPSK1C) c2(NP_036530) c3(phosphatidylinositol 4-phosphate 5-kinase type-l gamma isoform 2) c4(174264) c5(174265, 174266, 174267, 174268, 174269, 174270) c6( ) c7(crl); #c1(PIPSK1C) c2(XP_011526149) c3 (phosphatidylinositol 4-phosphate 5-kinase type-l gamma isoform X3) c4(320413) c5(320414, 320415, 320416, 320417, 320418, 320419, 320420) c6( ) c7(crl); #c1(PIPSKL1) c2(NP_001128691) c3(phosphatidylinositol 4-phosphate 5-kinase-like protein 1 isoform 1) c4(50545, 50546) c5(50547, 50548, 50549, 50550, 50551, 50552, 50553) c6( ) c7(T); #c1(PIPSKL1) c2(NP_775763) c3(phosphatidylinositol 4-phosphate 5-kinase-like protein 1 isoform 2) c4( ) c5(250217, 250218, 250219, 250220, 250221) c6( ) c7(T); #c1(PIPOX) c2(NP_057602) c3(peroxisomal sarcosine oxidase) c4(191572, 191573) c5(191574, 191575, 191576, 191577, 191578, 191579, 191580, 191581, 191582, 191583, 191584, 191585) c6( ) c7(aW, cr, aT, mE, aT, ad); #c1(PIR) c2(NP_003653) c3(pirin) c4(139984) c5(139985, 139986, 139987, 139988, 139989, 139990, (39991, 139992, 139993, 139994) c6( ) c7(LB, aT, fe, ji, jj, y, bt, kS); #c1(PISD) c2(XP_005261512) c3(phosphatidylserine decarboxylase proenzyme isoform X2) c4(277403, 277404) c5(277405, 277406, 277407, 277408, 277409, 277410, 277411, 277412, 277413, 277414, 277415, 277416) c6( ) c7(iQ, rz, bO, Ko, iV, he, bN); #c1(PISD) c2(XP_005261517) c3(phosphatidylserine decarboxylase proenzyme isoform X1) c4(277417, 277418, 277419, 277420, 277421, 277422) c5(277423, 277424, 277425, 277426, 277427, 277428, 277429, 277430, 277431, 277432, 277433, 277434, 277435, 277436, 277437, 277438, 277439, 277440, 277441, 277442, 277443, 277444, 277445, 277446, 277447, 277448, 277449, 277450, 277451, 277452, 277453, 277454, 277455, 277456, 277457, 277458, 277459, 277460, 277461, 277462, 277463, 277464, 277465, 277466) c6( ) c7(iQ, rz, bO, Ko, iV, he, bN); #c1(PITPNA) c2(NP_006215) c3(phosphatidylinositol transfer protein alpha isoform) c4(162983, 162984, 162985) c5(162986, 162987, 162988, 162989, 162990, 162991, 162992, 162993, 162994, 162995, 162996, 162997, 162998) c6( ) c7(fi); #c1(PITPNA) c2(XP_011522242) c3(phosphatidylinositol transfer protein alpha isoform isoform X2) c4( ) c5(316877, 316878, 316879, 316880, 316881) c6( ) c7(fi); #c1(PITPNM1) c2(NP_001124320) c3(membrane-associated phosphatidylinositol transfer protein 1 isoform b) c4(48660) c5(48661, 48662, 48663, 48664, 48665, 48666) c6( ) c7(nw); #c1(PITPNM1) c2(NP_004901) c3(membrane-associated phosphatidylinositol transfer protein 1 isoform a) c4(150645) c5(150646, 150647, 150648, 150649, 150650, 150651, 150652, 150653, 150654, 150655) c6( ) c7(nw); #c1(PITPNM3) c2(NP_001159438) c3(membrane-associated phosphatidylinositol transfer protein 3 isoform 2) c4(64044) c5(64045, 64046, 64047, 64048, 64049, 64050) c6( ) c7(bGe, lR, aW, nx, aT, nw, bfR, nC); #c1(PITPNM3) c2(NP_112497) c3(membrane-associated phosphatidylinositol transfer protein 3 isoform 1) c4(221180) c5(221181, 221182, 221183, 221184, 221185, 221186) c6( ) c7(bGe, lR, aW, nx, aT, nw, bfR, nC); #c1(PITRM1) c2(NP_001229236) c3(presequence protease, mitochondrial isoform 1 precursor) c4(84002) c5(84003, 84004, 84005, 84006, 84007, 84008) c6( ) c7(ac, btY, aoj, dy, alv, fq); #c1(PITRM1) c2(NP_001229238) c3(presequence protease, mitochondrial isoform 3 precursor) c4(84009, 84010, 84011) c5(84012, 84013, 84014, 84015, 84016, 84017, 84018, 84019, 84020, 84021, 84022, 84023) c6( ) c7(ac, btY, aoj, dy, alv, fq); #c1(PITRM1) c2(NP_055704) c3(presequence protease, mitochondrial isoform 2 precursor) c4(182924, 182925, 182926, 182927) c5(182928, 182929, 182930, 182931, 182932, 182933, 182934, 182935, 182936, 182937, 182938, 182939, 182940, 182941, 182942, 182943, 182944, 182945, 182946, 182947, 182948, 182949, 182950, 182951) c6( ) c7(ac, btY, aoj, dy, alv, fq); #c1(PITRM1) c2(XP_006717425) c3(presequence protease, mitochondrial isoform X3) c4(291070) c5(291071, 291072, 291073, 291074, 291075, 291076, 291077, 291078, 291079, 291080, 291081, 291082) c6( ) c7(ac, btY, aoj, dy, alv, fq); #c1(PITX1) c2(NP_002644) c3(pituitary homeobox 1) c4(130671, 130672, 130673) c5(130674, 130675, 130676, 130677, 130678, 130679, 130680, 130681, 130682, 130683, 130684, 130685, 130686, 130687, 130688, 130689, 130690, 130691, 130692, 130693, 130694, 130695, 130696) c6( ) c7(T, hl, pf, jx, crm, aA, fr, eA, ad, XW, ds, aT, bl, brQ, ae, Yi, yo, akb, s, aW, Xg, g, as, iV, cm, bz, Ym, atb, bN, aDQ, Ld, iQ, crn, hR, rx, aDR, B); #c1(PITX2) c2(NP_000316) c3 (pituitary homeobox 2 isoform c) c4(6998) c5(6999, 7000, 7001, 7002, 7003, 7004, 7005) c6( ) c7(T, BC, BD, mr, lT, wL, e, aw, bbH, cz, fd, n, crp, aln, afv, aml, aSL, hF, q, cd, ad, cro, hd, anA, any, bki, aT, dl, K, anz, an, rz, ae, eL, Lu, yo, s, aW, oV, xs, Az, r, atf, aq, ir, pT, dw, Vk, cM, t, dr, ck, es, bF, OG, ac, eM, nB, iA, PV, np, m, bkE, bD, gT, it, B); #c1(PITX2) c2(NP_700475) c3(pituitary homeobox 2 isoform b) c4(245937, 245938) c5(245939, 245940, 245941, 245942, 245943, 245944, 245945, 245946, 245947, 245948, 245949, 245950, 245951, 245952, 245953, 245954) c6( ) c7(T, BC, BD, mr, lT, wL, e, aw, bbH, cz, fd, n, crp, aLn, afv, aml, aSL, hF, q, cd, ad, cro, hd, anA, any, bki, aT, dL, K, anz, an, rz, ae, eL, Lu, yo, s, aW, oV, xs, Az, r, atf, aq, ir, pT, dw, Vk, cM, t, dr, ck, es, bF, OG, ac, eM, nB, iA, PV, np, m, bkE, bD, gT, it, B); #c1(PITX2) c2(NP_700476) c3(pituitary homeobox 2 isoform a) c4(245955, 245956) c5(245957, 245958, 245959, 245960, 245961, 245962, 245963, 245964, 245965, 245966, 245967, 245968) c6( ) c7(T, BC, BD, mr, lT, wL, e, aw, bbH, cz, fd, n, crp, aLn, afv, aml, aSL, hF, q, cd, ad, cro, hd, anA, any, bki, aT, dL, K, anz, an, rz, ae, eL, Lu, yo, s, aW, oV, xs, Az, r, atf, aq, ir, pT, dw, Vk, cM, t, dr, ck, es, bF, OG, ac, eM, nB, iA, PV, np, m, bkE, bD, gT, it, B); #c1(PITX3) c2(NP_005020) c3 (pituitary homeobox 3) c4( ) c5(152236, 152237, 152238, 152239, 152240, 152241, 152242, 152243) c6( ) c7(T, dD, ea, crq, v, eq, aBl, aSL, eA, asY, K, an, rz, af, hm, qE, yo, kj, aQp, aiV, kT, au, at, wR, bas, y, bLi, ao); #c1(PITX3) c2(XP_011538167) c3(pituitary homeobox 3 isoform X1) c4(335799) c5(335800, 335801, 335802) c6( ) c7(T, dD, ea, crq, v, eq, aBl, aSL, eA, asY, K, an, rz, af, hm, qE, yo, kj, alp, aiV, kT, au, at, wR, bas, y, bLi, ao); #c1(PIWIL1) c2(NP_001177900) c3 (piwi-like protein 1 isoform 2) c4(73122, 73123, 73124) c5( ) c6( ) c7(T, gU, wu, aA, fr, ci, eJ, q, hX, aT, vH, wm, B, ND, aW, r, aF, t, bz, es, m, lB, ne); #c1(PIWIL1) c2(XP_005253689) c3 (piwi-like protein 1 isoform X2) c4(272623) c5(272624, 272625, 272626, 272627, 272628, 272629) c6( ) c7(T, gU, wu, aA, fr, ci, eJ, q, hX, aT, vH, wm, B, ND, aW, r, aF, t, bz, es, m, lB, ne); #c1(PIWIL1) c2(XP_011544923) c3(piwi-like protein 1 isoform X3) c4(343883) c5(343884, 343885, 343886, 343887, 343888, 343889, 343890, 343891, 343892, 343893, 343894, 343895, 343896, 343897, 343898, 343899, 343900, 343901, 343902, 343903, 343904, 343905, 343906, 343907) c6( ) c7(T, gU, wu, aA, fr, ci, eJ, q, hX, aT, vH, wm, B, ND, aW, r, aF, t, bz, es, m, lB, ne); #c1(PIWIL2) c2(NP_001129193) c3(piwi-like protein 2) c4(51127) c5(51128, 51129, 51130, 51131, 51132, 51133, 51134, 51135, 51136, 51137) c6( ) c7(T, gU, ND, aW, if, hL, aT, vH, hQ, dL, ci, bu, eL, cl); #c1(PIWIL3) c2(NP_001008496) c3(piwi-like protein 3 isoform 1) c4(23266) c5(23267, 23268, 23269, 23270, 23271, 23272) c6( ) c7(vH, ND); #c1(PIWIL4) c2(NP_689644) c3(piwi-like protein 4) c4(243411) c5(243412, 243413, 243414, 243415, 243416, 243417) c6( ) c7(T, ND, wu, if, vH, wm, qN, B); #c1(PJA1) c2(NP_071763) c3(E3 ubiquitin-protein ligase Praja-1 isoform c) c4(211998) c5(211999, 212000, 212001, 212002, 212003, 212004) c6( ) c7(nf, dY, bjz); #c1(PJA1) c2(NP_660095) c3(E3 ubiquitin-protein ligase Praja-1 isoform a) c4(240356, 240357, 240358) c5(240359, 240360, 240361, 240362, 240363, 240364, 240365, 240366, 240367, 240368, 240369, 240370, 240371, 240372, 240373, 240374, 240375, 240376, 240377, 240378, 240379, 240380) c6( ) c7(nf, dY, bjz); #c1(PJA2) c2(NP_055634) c3(E3 ubiquitin-protein ligase Praja-2) c4(182407) c5(182408, 182409, 182410, 182411, 182412, 182413, 182414, 182415, 182416, 182417) c6( ) c7(T, hd, nM, nB); #c1(PKD1) c2(NP_000287) c3(polycystin-1 isoform 2 precursor) c4(6467) c5(6468, 6469, 6470, 6471, 6472, 6473, 6474) c6( ) c7(bL, T, hl, vl, eR, arS, aw, aA, cz, im, aj, v, fo, ci, Vz, fl, aY, cd, J, ad, aDF, aE, lg, Db, ds, arV, aT, rX, crs, us, K, Nj, aDZ, Xh, on, ae, d, aW, Ls, eH, dq, crr, aF, eF, axp, cru, dr, crv, qe, crt, wB, fP, uo, lO, aS, dy, ER, ei, bD, y, BV, jr); #c1(PKD1) c2(NP_001009944) c3(polycystin-1 isoform 1 precursor) c4(23769) c5(23770, 23771, 23772, 23773, 23774, 23775) c6( ) c7(bL, T, hl, vl, eR, arS, aw, aA, cz, im, aj, v, fo, ci, Vz, fL, aY, cd, J, ad, aDF, aE, lg, Db, ds, arV, aT, rX, crs, us, K, Nj, aDZ, Xh, on, ae, d, aW, Ls, eH, dq, crr, aF, eF, axp, cru, dr, crv, qe, crt, wB, fP, uo, lO, aS, dy, ER, ei, bD, y, BV, jr); #c1(PKD1) c2(XP_005255427) c3(polycystin-1 isoform X14) c4( ) c5(273638, 273639, 273640, 273641, 273642, 273643, 273644) c6( ) c7(bL, T, hl, vl, eR, arS, aw, aA, cz, im, aj, v, fo, ci, Vz, fl, aY, cd, J, ad, aDF, aE, lg, Db, ds, arV, aT, rX, crs, us, K, Nj, aDZ, Xh, on, ae, d, aW, Ls, eH, dq, crr, aF, eF, axp, cru, dr, crv, qe, crt, wB, fP, uo, lO, aS, dy, ER, ei, bD, y, BV, jr); #c1(PKD1) c2(XP_011520827) c3(polycystin-1 isoform X1) c4( ) c5(315817, 315818, 315819) c6( ) c7(bL, T, hl, vl, eR, arS, aw, aA, cz, im, aj, v, fo, ci, Vz, fl, aY, cd, J, ad, aDF, aE, lg, Db, ds, arV, aT, rX, crs, us, K, Nj, aDZ, Xh, on, ae, d, aW, Ls, eH, dq, crr, aF, eF, axp, cru, dr, crv, qe, crt, wB, fP, uo, lO, aS, dy, ER, ei, bD, y, BV, jr); #c1(PKD1) c2(XP_011520828) c3(polycystin-1 isoform X2) c4( ) c5(315820, 315821) c6( ) c7(bL, T, hl, vl, eR, arS, aw, aA, cz, im, aj, v, fo, ci, Vz, fl, aY, cd, J, ad, aDF, aE, lg, Db, ds, arV, aT, rX, crs, us, K, Nj, aDZ, Xh, on, ae, d, aW, Ls, eH, dq, crr, aF, eF, axp, cru, dr, crv, qe, crt, wB, fP, uo, lO, aS, dy, ER, ei, bD, y, BV, jr); #c1(PKD1) c2(XP_011520831) c3(polycystin-1 isoform X5) c4( ) c5(315822, 315823, 315824, 315825, 315826, 315827) c6( ) c7(bL, T, hl, vL, eR, arS, aw, aA, cz, im, aj, v, fo, ci, Vz, fl, aY, cd, J, ad, aDF, aE, lg, Db, ds, arV, aT, rX, crs, us, K, Nj, aDZ, Xh, on, ae, d, aW, Ls, eH, dq, crr, aF, eF, axp, cru, dr, crv, qe, crt, wB, fP, uo, lO, aS, dy, ER, ei, bD, y, BV, jr); #c1(PKD1) c2(XP_011520839) c3(polycystin-1 isoform X13) c4(315828, 315829, 315830) c5( ) c6( ) c7(bL, T, hl, vl, eR, arS, aw, aA, cz, im, aj, v, fo, ci, Vz, fl, aY, cd, J, ad, aDF, aE, lg, Db, ds, arV, aT, rX, crs, us, K, Nj, aDZ, Xh, on, ae, d, aW, Ls, eH, dq, crr, aF, eF, axp, cru, dr, crv, qe, crt, wB, fP, uo, lO, aS, dy, ER, ei, bD, y, BV, jr); #c1(PKD1L1) c2(NP_612152) c3(polycystic kidney disease protein 1-like 1) c4(236093) c5(236094, 236095, 236096, 236097, 236098, 236099) c6( ) c7(on, AW); #c1(PKD1L1) c2(XP_011513465) c3 (polycystic kidney disease protein 1-like 1 isoform X1) c4(305724) c5(305725, 305726, 305727, 305728, 305729, 305730) c6( ) c7(on, AW); #c1(PKD1L2) c2(NP_001070248) c3(polycystic kidney disease protein 1-like 2 isoform c precursor) c4(34630, 34631) c5(34632, 34633, 34634, 34635, 34636, 34637, 34638, 34639, 34640, 34641, 34642, 34643) c6( ) c7(bl, alv, wB, Ak); #c1(PKD1L3) c2(NP_853514) c3(polycystic kidney disease protein 1-like 3 precursor) c4( ) c5(255142) c6( ) c7(alv, wB, ahM); #c1(PKD2) c2(NP_000288) c3(polycystin-2) c4(6475) c5(6476, 6477, 6478, 6479, 6480, 6481) c6( ) c7(eR, arS, bSp, uv, cz, bB, aY, cd, ad, eA, aE, arV, pX, rX, adm, K, Nj, brV, ae, d, lR, iU, ff, Az, Lc, en, dr, crv, iT, bE, mx, gX, adl, U, E, wB, nw, aS, Hb, dy, bD, fy, BV, us); #c1(PKD2) c2(XP_011530331) c3 (polycystin-2 isoform X2) c4(326179) c5( ) c6( ) c7(eR, arS, bSp, uv, cz, bB, aY, cd, ad, eA, aE, arV, pX, rX, adm, K, Nj, brV, ae, d, lR, iU, ff, Az, Lc, en, dr, crv, iT, bE, mx, gX, adl, U, E, wB, nw, aS, Hb, dy, bD, fy, BV, us); #c1(PKD2) c2(XP_011530332) c3(polycystin-2 isoform X3) c4( ) c5(326180, 326181, 326182, 326183, 326184, 326185, 326186, 326187, 326188, 326189) c6( ) c7(eR, arS, bSp, uv, cz, bB, aY, cd, ad, eA, aE, arV, pX, rX, adm, K, Nj, brV, ae, d, lR, iU, ff, Az, Lc, en, dr, crv, iT, bE, mx, gX, adl, U, E, wB, nw, aS, Hb, dy, bD, fy, BV, us); #c1(PKD2L1) c2(NP_001240766) c3(polycystic kidney disease 2-like 1 protein isoform 2) c4(88770) c5(88771, 88772, 88773, 88774, 88775, 88776) c6( ) c7(uk, aop, bE, aW, Y, wB, ahM, aT, rX, aA, bB, aF, aor, ci, fd, hE, EB, vk, MG); #c1(PKD2L1) c2(NP_057196) c3(polycystic kidney disease 2-like 1 protein isoform 1) c4(189442) c5(189443, 189444, 189445, 189446, 189447, 189448) c6( ) c7(uk, aop, bE, aW, Y, wB, ahM, aT, rX, aA, bB, aF, aor, ci, fd, hE, EB, vk, MG); #c1(PKD2L1) c2(XP_011538623) c3(polycystic kidney disease 2-like 1 protein isoform X1) c4(336365) c5(336366, 336367, 336368, 336369, 336370, 336371) c6( ) c7(uk, aop, bE, aW, Y, wB, ahM, aT, rX, aA, bB, aF, aor, ci, fd, hE, EB, vk, MG); #c1(PKD2L1) c2(XP_011538624) c3(polycystic kidney disease 2-like 1 protein isoform X2) c4(336372) c5(336373, 336374, 336375, 336376, 336377, 336378) c6( ) c7(uk, aop, bE, aW, Y, wB, ahM, aT, rX, aA, bB, aF, aor, ci, fd, hE, EB, vk, MG); #c1(PKDCC) c2(NP_612379) c3(extracellular tyrosine-protein kinase PKDCC precursor) c4(236313) c5(236314, 236315, 236316, 236317, 236318, 236319) c6( ) c7(Hr, an); #c1(PKDREJ) c2(NP_006062) c3(polycystic kidney disease and receptor for egg jelly-related protein precursor) c4(161561) c5(161562, 161563, 161564, 161565, 161566, 161567) c6( ) c7(wB); #c1(PKHD1) c2(NP_733842) c3(fibrocystin isoform 2 precursor) c4(247233) c5(247234, 247235, 247236, 247237, 247238, 247239, 247240, 247241, 247242, 247243, 247244, 247245) c6( ) c7(blk, crw, c, arV, hl, EQ, r, cz, cd, wB, dy, aY, q, ad); #c1(PKHD1) c2(XP_011512982) c3(fibrocystin isoform X1) c4(304441, 304442) c5(304443, 304444, 304445, 304446, 304447, 304448) c6( ) c7(blk, crw, c, arV, hl, EQ, r, cz, cd, wB, dy, aY, q, ad); #c1(PKHD1) c2(XP_011512991) c3(fibrocystin isoform X10) c4( ) c5(304449) c6( ) c7(blk, crw, c, arV, hl, EQ, r, cz, cd, wB, dy, aY, q, ad); #c1(PKlA) c2(NP_006814) c3(cAMP-dependent protein kinase inhibitor alpha) c4(168325) c5(168326, 168327, 168328, 168329, 168330, 168331, 168332, 168333, 168334, 168335, 168336, 168337, 168338, 168339) c6( ) c7(aw, bK); #c1(PKIB) c2(NP_001257323) c3(cAMP-dependent protein kinase inhibitor beta isoform 2) c4(95144, 95145, 95146) c5(95147, 95148, 95149, 95150, 95151, 95152, 95153, 95154, 95155, 95156, 95157, 95158, 95159, 95160, 95161, 95162, 95163) c6( ) c7(aW, ae, ad, aT); #c1(PKIB) c2(NP_115860) c3(cAMP-dependent protein kinase inhibitor beta isoform 1) c4( ) c5(224355, 224356, 224357, 224358, 224359, 224360, 224361, 224362, 224363, 224364, 224365, 224366, 224367, 224368, 224369, 224370, 224371) c6( ) c7(aW, ae, ad, aT); #c1(PKLR) c2(NP_000289) c3(pyruvate kinase PKLR isoform 1) c4(6482, 6483, 6484) c5( ) c6( ) c7(byD, bL, T, os, Dw, eW, jx, jh, aj, dG, oZ, wy, hF, V, mC, aR, J, ad, qC, av, x, aaF, F, aE, rJ, ds, oW, aT, cU, lG, dV, hn, h, K, bq, ae, af, Rw, d, aW, Y, ff, eH, eZ, pR, lo, aq, VE, S, en, CQ, eF, cm, bz, dS, ck, rR, c, eM, crx, wB, U, cj, rk, k, fL, Cl, B, EB, L); #c1(PKLR) c2(NP_870986) c3(pyruvate kinase PKLR isoform 2) c4(255870) c5(255871, 255872, 255873, 255874, 255875, 255876) c6( ) c7(byD, bL, T, os, Dw, eW, jx, jh, aj, dG, oZ, wy, hF, V, mC, aR, J, ad, qC, av, x, aaF, F, aE, rJ, ds, oW, aT, cU, lG, dV, hn, h, K, bq, ae, af, Rw, d, aW, Y, ff, eH, eZ, pR, lo, aq, VE, S, en, CQ, eF, cm, bz, dS, ck, rR, c, eM, crx, wB, U, cj, rK, k, fL, Cl, B, EB, L); #c1 (PKLR) c2(XP_005245323) c3(pyruvate kinase PKLR isoform X1) c4( ) c5(266470, 266471, 266472, 266473, 266474, 266475, 266476, 266477, 266478, 266479, 266480, 266481, 266482) c6( ) c7(byD, bL, T, os, Dw, eW, jx, jh, aj, dG, oZ, wy, hF, V, mC, aR, J, ad, qC, av, x, aaF, F, aE, rJ, ds, oW, aT, cU, lG, dV, hn, h, K, bq, ae, af, Rw, d, aW, Y, ff, eH, eZ, pR, lo, aq, VE, S, en, CQ, eF, cm, bz, dS, ck, rR, c, eM, crx, wB, U, cj, rK, k, fL, Cl, B, EB, L); #c1(PKLR) c2(XP_011545234) c3(pyruvate kinase PKLR isoform X4) c4( ) c5(344028, 344029, 344030, 344031, 344032) c6( ) c7(byD, bL, T, os, Dw, eW, jx, jh, aj, dG, oZ, wy, hF, V, mC, aR, J, ad, qC, av, x, aaF, F, aE, rJ, ds, oW, aT, cU, lG, dV, hn, h, K, bq, ae, af, Rw, d, aW, Y, ff, eH, eZ, pR, lo, aq, VE, S, en, CQ, eF, cm, bz, dS, ck, rR, c, eM, crx, wB, U, cj, rK, k, fL, Cl, B, EB, L); #c1(PKM) c2(NP_001193725) c3(pyruvate kinase PKM isoform c) c4( ) c5(82499, 82500) c6( ) c7(byD, T, ew, aA, bB, ci, zP, q, eA, av, ft, yt, BJ, aT, K, bA, zQ, d, B, aW, yL, ff, r, fe, aq, af, S, en, g, t, bz, eM, bE, mf, m, qF, X); #c1(PKM) c2(NP_001193726) c3(pyruvate kinase PKM isoform d) c4(82501) c5(82502, 82503, 82504, 82505, 82506, 82507) c6( ) c7(byD, T, ew, aA, bB, ci, zP, q, eA, av, ft, yt, BJ, aT, K, bA, zQ, d, B, aW, yL, ff, r, fe, aq, aF, S, en, g, t, bz, eM, bE, mf, m, qF, X); #c1(PKM) c2(NP_001193727) c3 (pyruvate kinase PKM isoform e) c4(82508) c5(82509, 82510, 82511, 82512, 82513, 82514) c6( ) c7(byD, T, ew, aA, bB, ci, zP, q, eA, av, ft, yt, BJ, aT, K, bA, zQ, d, B, aW, yL, ff, r, fe, aq, aF, S, en, g, t, bz, eM, bE, mf, m, qF, X); #c1(PKM) c2(NP_001193728) c3(pyruvate kinase PKM isoform f) c4( ) c5(82515, 82516, 82517, 82518, 82519, 82520, 82521, 82522) c6( ) c7(byD, T, ew, aA, bB, ci, zP, q, eA, av, ft, yt, BJ, aT, K, bA, zQ, d, B, aW, yL, ff, r, fe, aq, aF, S, en, g, t, bz, eM, bE, mf, m, qF, X); #c1(PKM) c2(NP_002645) c3(pyruvate kinase PKM isoform a) c4( ) c5(130697, 130698, 130699, 130700, 130701) c6( ) c7(byD, T, ew, aA, bB, ci, zP, q, eA, av, ft, yt, BJ, aT, K, bA, zQ, d, B, aW, yL, ff, r, fe, aq, aF, S, en, g, t, bz, eM, bE, mf, m, qF, X); #c1(PKM) c2(NP_872270) c3(pyruvate kinase PKM isoform b) c4( ) c5(255983, 255984, 255985, 255986, 255987, 255988) c6( ) c7(byD, T, ew, aA, bB, ci, zP, q, eA, av, ft, yt, BJ, aT, K, bA, zQ, d, B, aW, yL, ff, r, fe, aq, aF, S, en, g, t, bz, eM, bE, mf, m, qF, X); #c1(PKM) c2(XP_005254500) c3(pyruvate kinase PKM isoform X3) c4( ) c5(273231) c6( ) c7(byD, T, ew, aA, bB, ci, zP, q, eA, av, ft, yt, BJ, aT, K, bA, zQ, d, B, aW, yL, ff, r, fe, aq, aF, S, en, g, t, bz, eM, bE, mf, m, qF, X); #c1(PKM) c2(XP_011519972) c3(pyruvate kinase PKM isoform X4) c4(314827) c5( ) c6( ) c7(byD, T, ew, aA, bB, ci, zP, q, eA, av, ft, yt, BJ, aT, K, bA, zQ, d, B, aW, yL, ff, r, fe, aq, aF, S, en, g, t, bz, eM, bE, mf, m, qF, X); #c1(PKM) c2(XP_011519975) c3 (pyruvate kinase PKM isoform X7) c4(314828) c5( ) c6( ) c7(byD, T, ew, aA, bB, ci, zP, q, eA, av, ft, yt, BJ, aT, K, bA, zQ, d, B, aW, yL, ff, r, fe, aq, aF, S, en, g, t, bz, eM, bE, mf, m, qF, X); #c1(PKMYT1) c2(NP_001245379) c3(membrane-associated tyrosine- and threonine-specific cdc2-inhibitory kinase isoform 3) c4(93358) c5(93359, 93360, 93361, 93362, 93363, 93364) c6( ) c7(he, atC, py, Y); #c1(PKMYT1) c2(NP_872629) c3(membrane-associated tyrosine- and threonine-specific cdc2-inhibitory kinase isoform 2) c4(256558, 256559) c5(256560, 256561, 256562, 256563, 256564, 256565, 256566, 256567, 256568, 256569, 256570, 256571) c6( ) c7(he, atC, py, Y); #c1(PKMYT1) c2(XP_011521036) c3(membrane-associated tyrosine- and threonine-specific cdc2-inhibitory kinase isoform X1) c4(315984, 315985, 315986, 315987) c5(315988, 315989, 315990, 315991, 315992, 315993, 315994, 315995, 315996, 315997, 315998, 315999, 316000) c6( ) c7(he, atC, py, Y); #c1(PKMYT1) c2(XP_011521038) c3(membrane-associated tyrosine- and threonine-specific cdc2-inhibitory kinase isoform X2) c4( ) c5(316001, 316002, 316003, 316004, 316005, 316006, 316007, 316008, 316009, 316010, 316011) c6( ) c7(he, atC, py, Y); #c1(PKN1) c2(NP_002732) c3(serine/threonine-protein kinase Nl isoform 2) c4(131521) c5(131522, 131523, 131524, 131525, 131526, 131527) c6( ) c7(T, afa, jh, bB, n, fr, ci, q, ad, hX, bv, hd, ib, aoc, Ot, aT, h, cy, By, hP, K, nJ, ae, d, ZH, s, CN, aW, Y, ff, xs, r, aq, agF, en, cl, g, t, kT, cT, Ew, c, nB, bE, eM, iA, dO, U, hL, nw, aS, m, mf, bnl, B, y); #c1(PKN1) c2(NP_998725) c3(serine/threonine-protein kinase Nl isoform 1) c4(265790) c5(265791, 265792, 265793, 265794, 265795, 265796) c6( ) c7(T, afa, jh, bB, n, fr, ci, q, ad, hX, bv, hd, ib, aoc, Ot, aT, h, cy, By, hP, K, nJ, ae, d, ZH, s, CN, aW, Y, ff, xs, r, aq, agF, en, cl, g, t, kT, cT, Ew, c, nB, bE, eM, iA, do, U, hL, nw, aS, m, mf, bnl, B, y); #c1(PKN2) c2(XP_011540071) c3(serine/threonine-protein kinase N2 isoform X1) c4( ) c5(338369, 338370, 338371, 338372, 338373, 338374) c6( ) c7(bL, ZH, CN, aoc, qt, aW, aT, aA, h, K, m, t, V, Pr); #c1(PKN2) c2(XP_011540073) c3(serine/threonine-protein kinase N2 isoform X4) c4(338375) c5(338376, 338377, 338378, 338379, 338380, 338381) c6( ) c7(bL, ZH, CN, aoc, qt, aW, aT, aA, h, K, m, t, V, Pr); #c1(PKN2) c2(XP_011540074) c3(serine/threonine-protein kinase N2 isoform X5) c4(338382, 338383) c5(338384, 338385, 338386, 338387, 338388, 338389, 338390, 338391, 338392, 338393, 338394, 338395, 338396, 338397, 338398, 338399, 338400, 338401) c6( ) c7(bL, ZH, CN, aoc, qt, aW, aT, aA, h, K, m, t, V, Pr); #c1(PKN3) c2(NP_037487) c3(serine/threonine-protein kinase N3) c4(175817) c5(175818, 175819, 175820, 175821, 175822, 175823) c6( ) c7(T); #c1(PKNOX1) c2(NP_001273187) c3(homeobox protein PKNOX1 isoform 2) c4(104519) c5(104520, 104521, 104522, 104523, 104524, 104525) c6( ) c7(ck, x, jh, Hn, jy, aj, aaw, cm, K, k, h, f, J); #c1(PKNOX1) c2(NP_004562) c3(homeobox protein PKNOX1 isoform 1) c4(147647, 147648) c5(147649, 147650, 147651, 147652, 147653, 147654, 147655, 147656, 147657, 147658, 147659, 147660, 147661) c6( ) c7(ck, x, jh, Hn, jy, aj, aaw, cm, K, k, h, f, J); #c1(PKNOX2) c2(XP_005271700) c3(homeobox protein PKNOX2 isoform X2) c4(283541) c5(283542, 283543, 283544, 283545, 283546, 283547, 283548, 283549, 283550, 283551, 283552, 283553, 283554, 283555, 283556, 283557) c6( ) c7(dS, lB, FT, ben); #c1(PKNOX2) c2(XP_011541247) c3(homeobox protein PKNOX2 isoform X1) c4(339867, 339868, 339869, 339870, 339871, 339872, 339873) c5(339874, 339875, 339876, 339877, 339878, 339879, 339880, 339881, 339882, 339883, 339884, 339885, 339886) c6( ) c7(dS, lB, FT, ben); #c1(PKNOX2) c2(XP_011541248) c3(homeobox protein PKNOX2 isoform X3) c4( ) c5(339887, 339888, 339889, 339890, 339891, 339892, 339893, 339894, 339895, 339896, 339897, 339898, 339899, 339900, 339901, 339902, 339903, 339904, 339905, 339906, 339907, 339908, 339909, 339910) c6( ) c7(dS, lB, FT, ben); #c1(PKP1) c2(NP_000290) c3(plakophilin-1 isoform Ib) c4(6485) c5(6486, 6487, 6488, 6489, 6490, 6491, 6492, 6493, 6494, 6495) c6( ) c7(bM, T, cc, bjA, pf, biw, ff, gZ, ww, lQ, en, aFJ, fq, B); #c1(PKP1) c2(NP_001005337) c3(plakophilin-1 isoform 1a) c4(21396) c5(21397, 21398, 21399, 21400, 21401, 21402) c6( ) c7(bM, T, cc, bjA, pf, biw, ff, gZ, ww, lQ, en, aFJ, fq, B); #c1(PKP2) c2(NP_001005242) c3(plakophilin-2 isoform 2a) c4(21237) c5(21238, 21239, 21240, 21241, 21242, 21243, 21244) c6( ) c7(cry, cc, QG, wK, cV, rf, gZ, ajM, biv, ci, g); #c1(PKP2) c2(NP_004563) c3(plakophilin-2 isoform 2b) c4(147662, 147663, 147664, 147665, 147666, 147667, 147668, 147669) c5(147670, 147671, 147672, 147673, 147674, 147675, 147676, 147677, 147678, 147679, 147680, 147681, 147682, 147683, 147684, 147685, 147686, 147687, 147688, 147689, 147690, 147691, 147692, 147693, 147694, 147695, 147696, 147697, 147698, 147699, 147700) c6( ) c7 (cry, cc, QG, wK, cV, rf, gZ, ajM, biv, ci, g); #c1(PKP3) c2(NP_009114) c3(plakophilin-3 isoform PKP3a) c4(170791) c5(170792, 170793, 170794, 170795, 170796, 170797) c6( ) c7(T, eM, gX, ci, bz, iu, ad); #c1(PKP4) c2(NP_001005476) c3(plakophilin-4 isoform b) c4(21727) c5(21728, 21729, 21730, 21731, 21732, 21733) c6( ) c7(en, agd, ht, ff, Ku); #c1(PKP4) c2(NP_001291900) c3(plakophilin-4 isoform e) c4( ) c5(116505, 116506, 116507, 116508, 116509, 116510) c6( ) c7(en, agd, ht, ff, Ku); #c1(PKP4) c2(NP_003619) c3(plakophilin-4 isoform a) c4(139668) c5(139669, 139670, 139671, 139672, 139673, 139674, 139675) c6( ) c7(en, agd, ht, ff, Ku); #c1(PKP4) c2(XP_011510322) c3 (plakophilin-4 isoform X4) c4(300886) c5( ) c6( ) c7(en, agd, ht, ff, Ku); #c1(PKP4) c2(XP_011510323) c3(plakophilin-4 isoform X5) c4(300887) c5( ) c6( ) c7(en, agd, ht, ff, Ku); #c1(PKP4) c2(XP_011510328) c3(plakophilin-4 isoform X9) c4( ) c5(300888, 300889, 300890, 300891, 300892) c6( ) c7(en, agd, ht, ff, Ku); #c1(PLA1A) c2(NP_001193889) c3(phospholipase A1 member A isoform 2 precursor) c4(83057) c5(83058, 83059, 83060, 83061, 83062, 83063) c6( ) c7(T, aw, Ak, BZ, zp, K, nA); #c1(PLA1A) c2(NP_001193890) c3(phospholipase A1 member A isoform 3) c4( ) c5(83064, 83065, 83066, 83067, 83068) c6( ) c7(T, aw, Ak, BZ, zp, K, nA); #c1(PLA1A) c2(NP_001280154) c3(phospholipase A1 member A isoform 4) c4(111462) c5(111463) c6( ) c7(T, aw, Ak, BZ, zp, K, nA); #c1(PLA1A) c2(NP_056984) c3(phospholipase A1 member A isoform 1 precursor) c4(188334) c5(188335, 188336, 188337, 188338, 188339, 188340) c6( ) c7(T, aw, Ak, BZ, zp, K, nA); #c1(PLA2G10) c2(NP_003552) c3(group 10 secretory phospholipase A2 precursor) c4(139058) c5(139059, 139060, 139061, 139062, 139063, 139064, 139065, 139066, 139067, 139068) c6( ) c7(DS, gh, T, bL, dD, bM, eR, ea, e, cR, bB, j, ct, eq, aY, q, aR, ad, bUp, av, Yc, aT, bK, K, ae, s, aW, ev, az, cr, r, aq, g, as, bz, zV, bF, eM, iT, boc, at, jM, y, B); #c1(PLAZG1ZA) c2(NP_110448) c3(group XIIA secretory phospholipase A2 precursor) c4(220433, 220434) c5(220435, 220436, 220437, 220438, 220439, 220440, 220441, 220442, 220443, 220444, 220445, 220446, 220447, 220448, 220449) c6(220450, 220451, 220452) c7(W); #c1(PLA2G12B) c2(NP_115951) c3(group X11B secretory phospholipase A2-like protein precursor) c4(224895) c5(224896, 224897, 224898, 224899, 224900, 224901) c6( ) c7(B); #c1(PLA2G15) c2(NP_036452) c3(group XV phospholipase A2 precursor) c4(173971) c5(173972, 173973, 173974, 173975, 173976, 173977, 173978, 173979, 173980, 173981, 173982, 173983, 173984, 173985, 173986, 173987, 173988, 173989, 173990, 173991, 173992, 173993, 173994, 173995) c6(173996, 173997, 173998, 173999) c7(T, rS, h, fr, j, hm); #c1(PLA2G15) c2(XP_011521281) c3(group XV phospholipase A2 isoform X1) c4(316175, 316176) c5( ) c6( ) c7(T, rS, h, fr, j, hm); #c1(PLA2G16) c2(NP_009000) c3(HRAS-like suppressor 3) c4(170/70) c5(170171, 170172, 170173, 170174, 170175, 170176, 170177, 170178, 170179, 170180) c6( ) c7(t, ci, m); #c1(PLAZG1B) c2(NP_000919) c3 (phospholipase A2 precursor) c4(17215, 17216) c5(17217, 17218, 17219, 17220, 17221, 17222) c6( ) c7(DS, gh, T, bM, aCh, ea, e, al, eb, ci, auu, aR, Vp, gJ, K, eN, hm, aa, Jl, aW, hJ, cm, bqd, U, m, Ro, P, NK, dD, Ht, aw, n, aj, ai, Yc, OV, fe, aq, kT, cA, ky, vb, FV, at, Pr, abW, B, bL, eR, dK, cz, fF, v, j, C, V, q, J, jF, axa, bUp, aT, bZk, cy, bK, aAt, ae, af, Pw, arl, ev, Zt, aAm, r, akY, cT, Yq, ac, nM, kB, gX, wR, mf, cS, EY, aan, fD, ha, blo, dG, eq, ad, hD, ji, cb, eZ, kD, iV, bz, bF, gk, bTF, z, ag, gZ, aS, y, xg, X); #c1(PLA2G3) c2(NP_056530) c3 (group 3 secretory phospholipase A2 precursor) c4(187964) c5(187965, 187966, 187967, 187968, 187969) c6( ) c7(dD, ac, bE, r, bB, h, eq, g, q); #c1(PLA2G3) c2(XP_011528505) c3(group 3 secretory phospholipase A2 isoform X1) c4( ) c5(323616) c6( ) c7(dD, ac, bE, r, bB, h, eq, g, q); #c1(PLAZG4A) c2(NP_077734) c3(cytosolic phospholipase A2) c4( ) c5(215322, 215323, 215324, 215325, 215326) c6( ) c7(oO, bL, T, dD, xu, bM, fR, sQ, DD, ea, fG, e, id, aw, fF, bB, dG, j, v, C, aXD, auu, eq, V, lR, q, aR, ad, ai, eA, axa, bUp, jz, ib, wD, lg, qp, aT, h, lG, cy, bK, K, lQ, ae, af, eu, B, xR, dH, iZ, aW, sp, Y, ff, r, fQ, dq, dN, aq, en, g, Z, as, fq, bz, pb, bN, ek, ou, mO, c, eM, bE, z, X, wB, uo, ut, fD, ei, y, bt, gq); #c1(PLA2G4A) c2(XP_011507945) c3(cytosolic phospholipase A2 isoform X1) c4(298291, 298292) c5(298293, 298294, 298295, 298296, 298297, 298298) c6( ) c7(oO, bL, T, dD, xu, bM, fR, sQ, DD, ea, fG, e, id, aw, fF, bB, dG, j, v, C, aXD, auu, eq, V, lR, q, aR, ad, ai, eA, axa, bUp, jz, ib, wD, lg, qp, aT, h, lG, cy, bK, K, lQ, ae, af, eu, B, xR, dH, iZ, aW, sp, Y, ff, r, fQ, dq, dN, aq, en, g, Z, as, fq, bz, pb, bN, eK, ou, m, c, eM, bE, z, X, wB, uo, ut, fD, ei, y, bt, gq); #c1(PLA2G4B) c2(NP_001108105) c3(cytosolic phospholipase A2 beta) c4(43099) c5(43100, 43101, 43102, 43103, 43104, 43105, 43106, 43107, 43108, 43109, 43110, 43111) c6(43112, 43113) c7(h); #c1(PLA2G4C) c2(NP_001152794) c3(cytosolic phospholipase A2 gamma isoform 2 precursor) c4(60179) c5(60180, 60181, 60182, 60183, 60184, 60185) c6( ) c7(h, t, z, eA); #c1(PLA2G4C) c2(NP_001152795) c3(cytosolic phospholipase A2 gamma isoform 3) c4( ) c5(60186, 60187, 60188, 60189, 60190, 60191) c6( ) c7(h, t, z, eA); #c1(PLAZG4C) c2(NP_003697) c3(cytosolic phospholipase A2 gamma isoform 1 precursor) c4(140234, 140235) c5(140236, 140237, 140238, 140239, 140240, 140241) c6( ) c7(h, t, z, eA); #c1(PLA2G4D) c2(NP_828848) c3(cytosolic phospholipase A2 delta) c4(253361) c5(253362, 253363, 253364, 253365, 253366, 253367) c6( ) c7(eE, ac, xR); #c1(PLA265) c2(XP_011539888) c3(calcium-dependent phospholipase A2 isoform X2) c4(338162) c5(338163) c6( ) c7(U, v, hl, crz, fR); #c1(PLA2G6) c2(NP_001186491) c3(85/88 kDa calcium-independent phospholipase A2 isoform b) c4(77703) c5(77704, 77705, 77706, 77707, 77708, 77709, 77710, 77711, 77712, 77713) c6( ) c7(gh, T, bM, aCh, ea, e, al, eb, ci, auu, aR, aU, K, eN, hm, aa, aW, hJ, cm, bqd, U, m, P, sc, dD, aw, aj, ai, crD, no, crB, dL, Ha, crC, OV, A, XT, KT, hk, vb, FV, at, Pr, abW, bL, ciD, dK, j, C, V, q, J, jF, axa, mj, aT, cy, an, ae, af, Pw, arl, Y, r, crA, t, cT, nM, ac, kB, au, wR, cS, EY, aan, bS, blo, dG, eq, ad, av, aV, hD, cb, lR, eZ, kD, bz, bF, gk, bTF, ag, gZ, Hs, aS, ei, X); #c1(PLA266) c2(NP_003551) c3(85/88 kDa calcium-independent phospholipase A2 isoform a) c4(139037, 139038, 139039) c5(139040, 139041, 139042, 139043, 139044, 139045, 139046, 139047, 139048, 139049, 139050, 139051, 139052, 139053, 139054, 139055, 139056, 139057) c6( ) c7(gh, T, bM, aCh, ea, e, al, eb, ci, auu, aR, aU, K, eN, hm, aa, aW, hJ, cm, bqd, U, m, P, sc, dD, aw, aj, ai, crD, no, crB, dL, Ha, crC, OV, A, XT, KT, hk, vb, FV, at, Pr, abW, bL, ciD, dK, j, C, V, q, J, jF, axa, mj, aT, cy, an, ae, af, Pw, arl, Y, r, crA, t, cT, nM, ac, kB, au, wR, cS, EY, aan, bS, blo, dG, eq, ad, av, aV, hD, cb, lR, eZ, kD, bz, bF, gk, bTF, ag, gZ, Hs, aS, ei, X); #c1(PLA2G6) c2(XP_005261828) c3(85/88 kDa calcium-independent phospholipase A2 isoform X5) c4(277636) c5(277637, 277638, 277639, 277640, 277641, 277642) c6( ) c7(gh, T, bM, aCh, ea, e, al, eb, ci, auu, aR, aU, K, eN, hm, aa, aW, hJ, cm, bqd, U, m, P, sc, dD, aw, aj, ai, crD, no, crB, dL, Ha, crC, OV, A, XT, KT, hk, vb, FV, at, Pr, abW, bL, ciD, dK, j, C, V, q, J, jF, axa, mj, aT, cy, an, ae, af, Pw, arl, Y, r, crA, t, cT, nM, ac, kB, au, wR, cS, EY, aan, bS, blo, dG, eq, ad, av, aV, hD, cb, lR, eZ, kD, bz, bF, gk, bTF, ag, gZ, Hs, aS, ei, X); #c1(PLA2G6) c2(XP_011528727) c3(85/88 kDa calcium-independent phospholipase A2 isoform X3) c4(324017) c5( ) c6( ) c7(gh, T, bM, aCh, ea, e, al, eb, ci, auu, aR, aU, K, eN, hm, aa, aW, hJ, cm, bqd, U, m, P, sc, dD, aw, aj, ai, crD, no, crB, dL, Ha, crC, OV, A, XT, KT, hk, vb, FV, at, Pr, abW, bL, ciD, dK, j, C, V, q, J, jF, axa, mj, aT, cy, an, ae, af, Pw, arl, Y, r, crA, t, cT, nM, ac, kB, au, wR, cS, EY, aan, bS, bLo, dG, eq, ad, av, aV, hD, cb, lR, eZ, kD, bz, bF, gk, bTF, ag, gZ, Hs, aS, ei, X); #c1(PLA2G6) c2(XP_011528728) c3(85/88 kDa calcium-independent phospholipase A2 isoform X4) c4(324018) c5( ) c6( ) c7(gh, T, bM, aCh, ea, e, al, eb, ci, auu, aR, aU, K, eN, hm, aa, aW, hJ, cm, bqd, U, m, P, sc, dD, aw, aj, ai, crD, no, crB, dL, Ha, crC, OV, A, XT, KT, hk, vb, FV, at, Pr, abW, bL, ciD, dK, j, C, V, q, J, jF, axa, mj, aT, cy, an, ae, af, Pw, arl, Y, r, crA, t, cT, nM, ac, kB, au, wR, cS, EY, aan, bS, bLo, dG, eq, ad, av, aV, hD, cb, lR, eZ, kD, bz, bF, gk, bTF, ag, gZ, Hs, aS, ei, X); #c1(PLA2G6) c2(XP_011528729) c3(85/88 kDa calcium-independent phospholipase A2 isoform X6) c4(324019) c5(324020, 324021, 324022, 324023, 324024, 324025) c6( ) c7(gh, T, bM, aCh, ea, e, al, eb, ci, auu, aR, aU, K, eN, hm, aa, aW, hJ, cm, bqd, U, m, P, sc, dD, aw, aj, ai, crD, no, crB, dL, Ha, crC, OV, A, XT, KT, hk, vb, FV, at, Pr, abW, bL, ciD, dK, j, C, V, q, J, jF, axa, mj, aT, cy, an, ae, af, Pw, arl, Y, r, crA, t, cT, nM, ac, kB, au, wR, cS, EY, aan, bS, blo, dG, eq, ad, av, aV, hD, cb, lR, eZ, kD, bz, bF, gk, bTF, ag, gZ, Hs, aS, ei, X); #c1(PLA2G6) c2(XP_011528730) c3(85/88 kDa calcium-independent phospholipase A2 isoform X7) c4( ) c5(324026, 324027, 324028, 324029, 324030, 324031, 324032, 324033, 324034, 324035, 324036, 324037) c6( ) c7(gh, T, bM, aCh, ea, e, al, eb, ci, auu, aR, aU, K, eN, hm, aa, aW, hJ, cm, bqd, U, m, P, sc, dD, aw, aj, ai, crD, no, crB, dL, Ha, crC, OV, A, XT, kT, hk, vb, FV, at, Pr, abW, bL, ciD, dK, j, C, V, q, J, jF, axa, mj, aT, cy, an, ae, af, Pw, arl, Y, r, crA, t, cT, nM, ac, kB, au, wR, cS, EY, aan, bS, blo, dG, eq, ad, av, aV, hD, cb, lR, eZ, kD, bz, bF, gk, bTF, ag, gZ, Hs, aS, ei, X); #c1(PLA2G7) c2(NP_005075) c3(platelet-activating factor acetylhydrolase precursor) c4(152641, 152642) c5(152643, 152644, 152645, 152646, 152647, 152648, 152649, 152650, 152651, 152652) c6( ) c7(bM, T, dD, ef, eR, aZ, aO, e, ea, aw, wL, cz, fF, mw, aj, jh, j, ah, eq, aLc, ad, nH, cc, ec, ql, lC, M, vc, ey, K, jo, bGD, ae, af, kl, anR, bf, A, cH, cW, sp, Yd, iV, wO, crE, ek, bJ, bF, mO, ac, dh, X, mx, lf, bm, cS, aS, ei, pJ, gq); #c1(PLA2G7) c2(XP_005249466) c3(platelet-activating factor acetylhydrolase isoform X2) c4(269432) c5(269433, 269434, 269435, 269436, 269437, 269438, 269439, 269440, 269441, 269442, 269443, 269444) c6( ) c7(bM, T, dD, ef, eR, aZ, aO, e, ea, aw, wL, cz, fF, mw, aj, jh, j, ah, eq, aLc, ad, nH, cc, ec, ql, lC, M, vc, ey, K, jo, bGD, ae, af, kl, anR, bf, A, CH, cW, sp, Yd, iV, wU, crE, ek, bJ, bF, mO, ac, dh, X, mx, lf, bm, cS, aS, ei, pJ, gq); #c1(PLAZR1) c2(NP_001007268) c3(secretory phospholipase A2 receptor isoform 2 precursor) c4(22657) c5(22658, 22659, 22660, 22661, 22662, 22663) c6( ) c7(ck, es, T, ac, iA, dK, bt, bw, fr, hF, lB); #c1(PLAZR1) c2(NP_031392) c3(secretory phospholipase A2 receptor isoform 1 precursor) c4(172195) c5(172196, 172197, 172198, 172199, 172200, 172201) c6( ) c7(ck, es, T, ac, iA, dK, bt, bw, fr, hF, lB); #c1(PLAA) c2(NP_001026859) c3(phospholipase A-2-activating protein) c4(29671) c5(29672, 29673, 29674, 29675, 29676, 29677) c6( ) c7(T, Dy, bE, hk, Y, Qu, wu, bB, h, fe, wm, nJ); #c1(PLAC1) c2(NP_068568) c3(placenta-specific protein 1 precursor) c4(209426, 209427) c5(209428, 209429, 209430, 209431, 209432, 209433, 209434, 209435, 209436, 209437, 209438, 209439) c6( ) c7(av, T, jv, aW, e, aT, eb, bu, uC); #c1(PLAC8) c2(NP_001124187) c3(placenta-specific gene 8 protein) c4(48468) c5(48469, 48470, 48471, 48472, 48473, 48474, 48475, 48476, 48477, 48478, 48479, 48480, 48481, 48482, 48483, 48484, 48485, 48486, 48487, 48488) c6( ) c7(T, bE, bB, ae, cd, ad); #c1(PLAG1) c2(NP_001108106) c3(zinc finger protein PLAG1 isoform a) c4(43114) c5(43115, 43116, 43117, 43118, 43119, 43120, 43121, 43122, 43123, 43124, 43125, 43126) c6( ) c7(gh, T, aiH, dA, WA, bkF, rQ, n, Fb, ci, eW, ad, afb, hW, aeX, aT, aHs, ji, ae, s, Ub, aW, cr, g, t, hq, ck, iO, QG, pu, GU, Jr, cj, hj, m, bt, LS); #c1(PLAG1) c2(NP_001108107) c3(zinc finger protein PLAG1 isoform b) c4(43127) c5(43128, 43129, 43130, 43131, 43132) c6( ) c7(gh, T, aiH, dA, WA, bkF, rQ, n, Fb, ci, eW, ad, afb, hW, aeX, aT, als, ji, ae, s, Ub, aW, cr, g, t, hq, ck, iO, QG, pu, GU, Jr, cj, hj, m, bt, LS); #c1(PLAGL1) c2(NP_001275966) c3(zinc finger protein PLAGL1 isoform 1) c4(107575, 107576) c5(107577, 107578, 107579, 107580, 107581, 107582, 107583, 107584, 107585, 107586, 107587, 107588, 107589, 107590, 107591, 107592, 107593, 107594, 107595, 107596, 107597) c6( ) c7(bL, T, kh, aNA, ml, aA, Bm, cz, aj, dG, ci, JW, ahy, mh, Rl, afQ, aT, de, yo, ZH, s, CN, aW, lW, LO, ff, hQ, en, g, t, iK, es, sS, crF, hR, m); #c1(PLAGL1) c2(NP_001275974) c3(zinc finger protein PLAGL1 isoform 2) c4(107598, 107599) c5(107600, 107601, 107602, 107603, 107604, 107605, 107606, 107607, 107608, 107609, 107610, 107611, 107612, 107613, 107614, 107615, 107616, 107617, 107618, 107619, 107620, 107621, 107622, 107623, 107624, 107625, 107626, 107627, 107628, 107629, 107630, 107631, 107632, 107633, 107634, 107635) c6( ) c7(bL, T, kh, aNA, ml, aA, Bm, cz, aj, dG, ci, JW, ahy, mh, Rl, afQ, aT, de, yo, ZH, s, CN, aW, lW, LO, ff, hQ, en, g, t, ik, es, sS, crF, hR, m); #c1(PLAGL2) c2(NP_002648) c3(zinc finger protein PLAGL2) c4(130702) c5(130703, 130704, 130705, 130706, 130707, 130708, 130709) c6( ) c7(ck, av, iu, h, bt, bz, crG); #c1(PLAT) c2(NP_000921) c3(tissue-type plasminogen activator isoform 1 preproprotein) c4(17223, 17224, 17225, 17226, 17227, 17228, 17229, 17230, 17231) c5(17232, 17233, 17234, 17235, 17236, 17237, 17238, 17239, 17240, 17241, 17242, 17243, 17244, 17245, 17246, 17247, 17248, 17249, 17250, 17251, 17252, 17253, 17254, 17255, 17256, 17257, 17258, 17259, 17260, 17261, 17262, 17263, 17264, 17265, 17266, 17267, 17268, 17269, 17270, 17271, 17272, 17273, 17274, 17275, 17276, 17277, 17278, 17279, 17280, 17281, 17282, 17283, 17284, 17285, 17286, 17287, 17288, 17289, 17290, 17291, 17292, 17293, 17294, 17295, 17296, 17297, 17298, 17299, 17300, 17301, 17302, 17303, 17304, 17305, 17306, 17307, 17308, 17309, 17310, 17311, 17312, 17313, 17314, 17315, 17316, 17317, 17318, 17319, 17320, 17321, 17322, 17323, 17324, 17325, 17326, 17327, 17328, 17329, 17330, 17331, 17332, 17333, 17334, 17335, 17336, 17337, 17338, 17339, 17340, 17341, 17342, 17343, 17344, 17345, 17346, 17347, 17348, 17349, 17350, 17351, 17352, 17353, 17354, 17355, 17356, 17357, 17358, 17359, 17360, 17361, 17362, 17363, 17364, 17365, 17366, 17367, 17368, 17369) c6(17370, 17371, 17372, 17373, 17374, 17375, 17376, 17377, 17378, 17379, 17380) c7(T, aC, fR, ea, e, fd, bB, ci, crK, aR, x, bv, M, DO, K, xa, hm, Ok, eG, Hl, aW, g, as, cm, bwe, uC, cj, uo, wx, mk, k, m, cho, zv, dD, sQ, atC, My, aw, RC, ez, cd, cc, vM, h, lG, hn, ux, fJ, fQ, aq, fe, hk, vb, hL, uz, te, ut, bni, cB, B, bL, jv, eR, atB, yk, ew, aA, cz, crl, j, C, V, eA, acj, uT, lg, HC, aT, cy, dz, j, ae, af, dl, eu, kl, bf, Qr, iZ, Y, r, lo, crH, en, cl, t, eL, ck, ajl, wg, ac, AH, bpB, dy, fD, kZ, crJ, sY, wi, iW, ha, jh, oR, eq, crl, ad, fU, lQ, xX, azS, et, ff, eZ, crM, aF, Hv, qa, ek, iq, bE, MJ, aS, bC, pk, y, kS); #c1(PLAT) c2(NP_127509) c3(tissue-type plasminogen activator isoform 3 preproprotein) c4(226569, 226570) c5(226571, 226572, 226573, 226574, 226575, 226576, 226577, 226578, 226579, 226580, 226581, 226582, 226583, 226584, 226585, 226586, 226587, 226588, 226589, 226590, 226591, 226592, 226593, 226594, 226595, 226596, 226597) c6(226598, 226599, 226600, 226601, 226602, 226603) c7(T, aC, fR, ea, e, fd, bB, ci, crK, aR, x, bv, M, DO, K, xa, hm, Ok, eG, Hl, aW, g, as, cm, bwe, uC, cj, uo, wx, mk, k, m, cho, zv, dD, sQ, atC, My, aw, RC, ez, cd, cc, vM, h, lG, hn, ux, fJ, fQ, aq, fe, hk, vb, hl, uz, te, ut, bni, cB, B, bL, jv, eR, atB, yk, ew, aA, cz, crL, j, C, V, eA, acj, uT, lg, HC, aT, cy, dz, j, ae, af, dl, eu, kl, bf, Qr, iZ, Y, r, lo, crH, en, cl, t, el, ck, ajl, wg, ac, AH, bpB, dy, fD, kZ, crJ, sY, wi, iW, ha, jh, oR, eq, crl, ad, fU, lQ, xX, azS, et, ff, eZ, crM, aF, Hv, qa, ek, iq, bE, MJ, aS, bC, pK, y, kS); #c1(PLAU) c2(NP_001138503) c3(urokinase-type plasminogen activator isoform 2) c4(56905) c5(56906, 56907, 56908, 56909, 56910, 56911) c6( ) c7(bM, T, aC, fR, ea, e, al, Xi, bB, axk, lR, ci, bRn, aY, ip, bv, dY, aE, K, cp, hm, eG, LC, aW, az, vs, g, as, BE, eF, uC, eM, nB, iA, iT, wx, mk, m, ik, Vj, dD, crN, aik, sw, wu, My, ht, aw, qy, wJ, n, BH, fr, uE, zP, fK, Lv, lG, h, dl, ey, ux, cY, wC, A, fQ, aq, kp, cM, sE, dB, eD, hL, at, amb, bni, cB, B, it, dA, eR, fG, Lx, jx, aA, fF, j, acn, Vh, V, q, eA, eL, fh, rS, qK, yg, aT, cy, ae, af, dl, bA, ne, eu, jf, kl, bf, s, nO, oV, Y, ev, bg, r, en, cl, t, pb, ac, lV, nG, bpB, fD, bt, VB, ao, On, sY, kh, id, wi, dM, eq, bCx, ad, kL, dP, hX, bqs, av, afm, yt, sN, wm, aGh, d, ZE, anv, qt, ff, xs, eH, dN, crM, kD, aF, aFd, iR, bz, Hv, ek, LB, Hw, zr, bE, arE, dX, aS, XO, y, we, X); #c1(PLAU) c2(XP_011538168) c3(urokinase-type plasminogen activator isoform X1) c4(335803, 335804) c5(335805, 335806, 335807, 335808, 335809, 335810, 335811, 335812, 335813, 335814, 335815, 335816, 335817, 335818, 335819, 335820) c6( ) c7(bM, T, aC, fR, ea, e, al, Xi, bB, axk, lR, ci, bRn, aY, ip, bv, dY, aE, K, cp, hm, eG, LC, aW, az, vs, g, as, eE, eF, uC, eM, nB, iA, iT, wx, mk, m, ik, Vj, dD, crN, aik, sw, wu, My, ht, aw, qy, wJ, n, BH, fr, uE, zP, fK, Lv, lG, h, dl, ey, ux, cY, wC, A, fQ, aq, kp, cM, sE, dB, eD, hL, at, amb, bni, cB, B, it, dA, eR, fG, Lx, jx, aA, fF, j, acn, Vh, V, q, eA, eJ, fh, rS, qK, yg, aT, cy, ae, af, dl, bA, ne, eu, jf, kl, bf, s, nO, oV, Y, ev, bg, r, en, cl, t, pb, ac, lV, nG, bpB, fD, bt, VB, ao, On, sY, kh, id, wi, dM, eq, bCx, ad, kL, dP, hX, bqs, av, afm, yt, sN, wm, aGh, d, ZE, anv, qt, ff, xs, eH, dN, crM, KD, aF, aFd, iR, bz, Hv, ek, LB, Hw, zr, bE, arE, dX, aS, XO, y, we, X); #c1(PLAUR) c2(NP_001005376) c3 (urokinase plasminogen activator surface receptor isoform 2 precursor) c4(21589) c5(21590, 21591, 21592, 21593, 21594, 21595) c6( ) c7(bM, sY, T, jv, dD, fR, aC, id, fG, aw, aA, bB, n, eq, ci, bX, lR, fr, q, ad, eA, ip, av, ft, aE, yt, aT, h, dL, pm, in, rz, ae, kl, d, B, aW, sp, Y, eH, ff, r, dN, kp, aF, en, mo, g, t, uC, bz, pb, eE, bN, eF, ar, iQ, ac, nB, bE, at, aS, m, dy, bt, cB, X); #c1(PLAUR) c2(NP_001005377) c3(urokinase plasminogen activator surface receptor isoform 3 precursor) c4(21596) c5(21597, 21598, 21599, 21600, 21601, 21602) c6( ) c7(bM, sY, T, jv, dD, fR, aC, id, fG, aw, aA, bB, n, eq, ci, bX, lR, fr, q, ad, eA, ip, av, ft, aE, yt, aT, h, dl, pm, in, rz, ae, kl, d, B, aW, sp, Y, eH, ff, r, dN, kp, aF, en, mo, g, t, uC, bz, pb, eE, bN, eF, ar, iQ, ac, nB, bE, at, aS, m, dy, bt, cB, X); #c1(PLAUR) c2(NP_002650) c3(urokinase plasminogen activator surface receptor isoform 1 precursor) c4(130710) c5(130711, 130712, 130713, 130714, 130715, 130716) c6( ) c7(bM, sY, T, jv, dD, fR, aC, id, fG, aw, aA, bB, n, eq, ci, bX, lR, fr, q, ad, eA, ip, av, ft, aE, yt, aT, h, dL, pm, in, rz, ae, kl, d, B, aW, sp, Y, eH, ff, r, dN, kp, aF, en, mo, g, t, uC, bz, pb, eE, bN, eF, ar, iQ, ac, nB, bE, at, aS, m, dy, bt, cB, X); #c1(PLB1) c2(NP_001164056) c3(phospholipase B1, membrane-associated isoform 2 precursor) c4(66556) c5(66557, 66558, 66559, 66560, 66561, 66562) c6( ) c7(DS, bL, NK, T, dD, bM, eR, DD, Ht, ea, dK, e, al, aw, fF, aj, dG, j, v, C, auu, eq, V, q, J, aR, ad, jF, axa, bUp, gJ, cq, ai, Yc, ib, aT, h, bZk, cy, ji, K, cb, ae, avn, af, Pw, aiV, dH, Jl, aW, ev, Zt, aAm, r, akY, aq, iV, cm, bz, ck, bF, gk, cT, Yq, ky, cA, iT, z, X, eB, U, at, mf, aS, EY, aan, m, CZ, eA, bt, xg, B, y, Ro); #c1(PLB1) c2(NP_694566) c3(phospholipase B1, membrane-associated isoform 1 precursor) c4(245284) c5(245285, 245286, 245287, 245288, 245289, 245290) c6( ) c7(DS, bL, NK, T, dD, bM, eR, DD, Ht, ea, dK, e, al, aw, fF, aj, dG, j, v, C, auu, eq, V, q, J, aR, ad, jF, axa, bUp, gJ, cq, ai, Yc, ib, aT, h, bZk, cy, ji, K, cb, ae, avn, af, Pw, aiV, dH, Jl, aW, ev, Zt, aAm, r, akY, aq, iV, cm, bz, ck, bF, gk, cT, Yq, ky, cA, iT, z, X, eB, U, at, mf, aS, EY, aan, m, CZ, eA, bt, xg, B, y, Ro); #c1(PLB1) c2(XP_011530907) c3(phospholipase B1, membrane-associated isoform X22) c4(326899) c5( ) c6( ) c7(DS, bL, NK, T, dD, bM, eR, DD, Ht, ea, dK, e, al, aw, fF, aj, dG, j, v, C, auu, eq, V, q, J, aR, ad, jF, axa, bUp, gJ, cq, ai, Yc, ib, aT, h, bZk, cy, ji, K, cb, ae, avn, af, Pw, aiV, dH, Jl, aW, ev, Zt, aAm, r, akY, aq, iV, cm, bz, ck, bF, gk, cT, Yq, ky, cA, iT, z, X, eB, U, at, mf, aS, EY, aan, m, CZ, eA, bt, xg, B, y, Ro); #c1(PLB1) c2(XP_011530910) c3(phospholipase B1, membrane-associated isoform X26) c4(326900) c5(326901, 326902, 326903, 326904, 326905, 326906) c6( ) c7(DS, bL, NK, T, dD, bM, eR, DD, Ht, ea, dK, e, al, aw, fF, aj, dG, j, v, C, auu, eq, V, q, J, aR, ad, jF, axa, bUp, gJ, cq, ai, Yc, ib, aT, h, bZk, cy, ji, K, cb, ae, avn, af, Pw, aiV, dH, Jl, aW, ev, Zt, aAm, r, akY, aq, iV, cm, bz, ck, bF, gk, cT, Yq, ky, cA, iT, z, X, eB, U, at, mf, aS, EY, aan, m, CZ, eA, bt, xg, B, y, Ro); #c1(PLB1) c2(XP_011530912) c3(phospholipase B1, membrane-associated isoform X27) c4( ) c5(326907, 326908, 326909, 326910, 326911, 326912) c6( ) c7(DS, bL, NK, T, dD, bM, eR, DD, Ht, ea, dK, e, al, aw, fF, aj, dG, j, v, C, auu, eq, V, q, J, aR, ad, jF, axa, bUp, gJ, cq, ai, Yc, ib, aT, h, bZk, cy, ji, K, cb, ae, avn, af, Pw, aiV, dH, Jl, aW, ev, Zt, aAm, r, akY, aq, iV, cm, bz, ck, bF, gk, cT, Yq, ky, cA, iT, z, X, eB, U, at, mf, aS, EY, aan, m, CZ, eA, bt, xg, B, y, Ro); #c1(PLBD1) c2(NP_079105) c3(phospholipase B-like 1 precursor) c4(217032) c5(217033, 217034, 217035, 217036, 217037, 217038, 217039, 217040) c6(217041, 217042) c7(eM); #c1(PLCB1) c2(NP_056007) c3(1-phosphatidylinositol 4,5-bisphosphate phosphodiesterase beta-1 isoform a) c4(185082, 185083) c5(185084, 185085, 185086, 185087, 185088, 185089, 185090, 185091, 185092, 185093, 185094, 185095, 185096) c6( ) c7(aw, ha, ht, bO, dP, aT, h, lm, bK, af, cC, crO, crP, aW, ff, en, bw, dR, ac, cA, ei, bt, X); #c1(PLCB1) c2(NP_877398) c3(1-phosphatidylinositol 4,5-bisphosphate phosphodiesterase beta-1 isoform b) c4(256740, 256741) c5(256742, 256743, 256744, 256745, 256746, 256747, 256748, 256749, 256750, 256751, 256752, 256753, 256754, 256755, 256756, 256757) c6( ) c7(aw, ha, ht, bO, dP, aT, h, lm, bK, af, cC, crO, crP, aW, ff, en, bw, dR, ac, cA, ei, bt, X); #c1(PLCB1) c2(XP_011527501) c3(1-phosphatidylinositol 4,5-bisphosphate phosphodiesterase beta-1 isoform X1) c4(322285) c5(322286, 322287, 322288, 322289, 322290, 322291) c6( ) c7(aw, ha, ht, bO, dP, aT, h, lm, bK, af, cC, crO, crP, aW, ff, en, bw, dR, ac, cA, ei, bt, X); #c1(PLCB1) c2(XP_011527502) c3(1-phosphatidylinositol 4,5-bisphosphate phosphodiesterase beta-1 isoform X2) c4(322292) c5( ) c6( ) c7(aw, ha, ht, bO, dP, aT, h, lm, bK, af, cC, crO, crP, aW, ff, en, bw, dR, ac, cA, ei, bt, X); #c1(PLCB1) c2(XP_011527504) c3(1-phosphatidylinositol 4,5-bisphosphate phosphodiesterase beta-1 isoform X4) c4( ) c5(322293, 322294, 322295, 322296, 322297, 322298) c6( ) c7(aw, ha, ht, bO, dP, aT, h, lm, bK, af, cC, crO, crP, aW, ff, en, bw, dR, ac, cA, ei, bt, X); #c1(PLCB1) c2(XP_011527505) c3(1-phosphatidylinositol 4,5-bisphosphate phosphodiesterase beta-1 isoform X5) c4(322299, 322300) c5(322301, 322302, 322303, 322304, 322305, 322306, 322307, 322308, 322309, 322310, 322311) c6( ) c7(aw, ha, ht, bO, dP, aT, h, lm, bk, af, cC, crO, crP, aW, ff, en, bw, dR, ac, cA, ei, bt, X); #c1(PLCB2) c2(NP_001271226) c3(1-phosphatidylinositol 4,5-bisphosphate phosphodiesterase beta-2 isoform 2) c4(103659) c5(103660, 103661, 103662, 103663, 103664, 103665) c6( ) c7(T, cF, kP, aW, aT, h, yA); #c1(PLCB2) c2(NP_004564) c3(1-phosphatidylinositol 4,5-bisphosphate phosphodiesterase beta-2 isoform 1) c4(147701) c5(147702, 147703, 147704, 147705, 147706, (47707) c6( ) c7(T, cF, kP, aW, aT, h, yA); #c1(PLCB3) c2(NP_001171812) c3(1-phosphatidylinositol 4,5-bisphosphate phosphodiesterase beta-3 isoform 2) c4(71825) c5(71826, 71827, 71828, 71829, 71830, 71831) c6( ) c7(pY, pX, aw, dN, ahJ, h, azg, ci, bq, eE); #c1(PLCB4) c2(NP_000924) c3(1-phosphatidylinositol 4,5-bisphosphate phosphodiesterase beta-4 isoform a) c4(17381, 17382) c5(17383, 17384, 17385, 17386, 17387, 17388, 17389, 17390, 17391, 17392) c6( ) c7(dP, cA, aW, crQ, Y, bO, aT, h, bw, bgd, ei, an, he, bt, fq); #c1(PLCB4) c2(NP_001166117) c3(1-phosphatidylinositol 4,5-bisphosphate phosphodiesterase beta-4 isoform c) c4(68225, 68226, 68227) c5(68228, 68229, 68230, 68231, 68232, 68233, 68234, 68235, 68236, 68237, 68238, 68239) c6( ) c7(dP, cA, aW, crl, Y, bO, aT, h, bw, bgd, ei, an, he, bt, fq); #c1(PLCB4) c2(NP_877949) c3(1-phosphatidylinositol 4,5-bisphosphate phosphodiesterase beta-4 isoform b) c4(256962) c5(256963, 256964, 256965, 256966, 256967, 256968, 256969, 256970, 256971, 256972) c6( ) c7(dP, cA, aW, crQ, Y, bO, aT, h, bw, bgd, ei, an, he, bt, fq); #c1(PLCB4) c2(XP_011527553) c3(1-phosphatidylinositol 4,5-bisphosphate phosphodiesterase beta-4 isoform X2) c4( ) c5(322376, 322377, 322378, 322379, 322380, 322381, 322382, 322383, 322384, 322385) c6( ) c7(dP, cA, aW, crl, Y, bO, aT, h, bw, bgd, ei, an, he, bt, fq); #c1(PLCB4) c2(XP_011527555) c3(1-phosphatidylinositol 4,5-bisphosphate phosphodiesterase beta-4 isoform X3) c4(322386, 322387) c5(322388, 322389, 322390, 322391, 322392, 322393, 322394, 322395, 322396, 322397, 322398, 322399, 322400, 322401) c6( ) c7(dP, cA, aW, crQ, Y, bO, aT, h, bw, bgd, ei, an, he, bt, fq); #c1(PLCD1) c2(NP_001124436) c3(1-phosphatidylinositol 4,5-bisphosphate phosphodiesterase delta-l isoform 1) c4(48858) c5(48859, 48860, 48861, 48862, 48863, 48864) c6( ) c7(eR, h, lu, crR, Bc, aW, ff, aT, Lv, alw, aA, cz, mw, ci, j, en, af, he, it, cd); #c1(PLCD1) c2(NP_006216) c3(1-phosphatidylinositol 4,5-bisphosphate phosphodiesterase delta-1 isoform 2) c4(162999) c5(163000, 163001, 163002, 163003, 163004, 163005) c6( ) c7(eR, h, lu, crR, Bc, aW, ff, aT, Lv, alw, aA, cz, mw, ci, j, en, af, he, it, cd); #c1(PLCD3) c2(NP_588614) c3(1-phosphatidylinositol 4,5-bisphosphate phosphodiesterase delta-3) c4(234817) c5(234818, 234819, 234820, 234821, 234822, 234823, 234824) c6( ) c7(dP, oe, h); #c1(PLCD4) c2(NP_116115) c3(1-phosphatidylinositol 4,5-bisphosphate phosphodiesterase delta-4) c4(225367, 225368, 225369) c5(225370, 225371, 225372, 225373, 225374, 225375, 225376, 225377, 225378, 225379, 225380, 225381, 225382, 225383, 225384, 225385, 225386, 225387, 225388, 225389, 225390, 225391) c6( ) c7(h, aW, aT); #c1(PLCD4) c2(XP_005246972) c3(1-phosphatidylinositol 4,5-bisphosphate phosphodiesterase delta-4 isoform X5) c4(267553) c5(267554, 267555, 267556, 267557, 267558, 267559, 267560, 267561, 267562, 267563, 267564, 267565, 267566, 267567, 267568, 267569, 267570) c6( ) c7(h, aW, aT); #c1(PLCD4) c2(XP_011510314) c3(1-phosphatidylinositol 4,5-bisphosphate phosphodiesterase delta-4 isoform X3) c4(300885) c5( ) c6( ) c7(h, aW, aT); #c1(PLCE1) c2(NP_001159451) c3(1-phosphatidylinositol 4,5-bisphosphate phosphodiesterase epsilon-1 isoform 2) c4(64137) c5(64138, 64139, 64140, 64141, 64142, 64143, 64144, 64145, 64146, 64147) c6( ) c7(T, eR, pf, aD, oe, ht, aA, cz, it, bZ, ah, ci, q, atP, cF, io, vw, h, hu, hB, af, WW, li, atJ, Jl, ff, gi, r, alw, kt, aDm, aF, en, g, iK, sk, bbz, ac, AH, gX, at, acJ, Pr); #c1(PLCE1) c2(NP_057425) c3(1-phosphatidylinositol 4,5-bisphosphate phosphodiesterase epsilon-1 isoform 1) c4(190738, 190739) c5(190740, 190741, 190742, 190743, 190744, 190745, 190746, 190747, 190748, 190749, 190750, 190751) c6( ) c7(T, eR, pf, aD, oe, ht, aA, cz, it, bZ, ah, ci, q, atP, cF, io, vw, h, hu, hB, af, WW, li, atJ, Jl, ff, gi, r, alw, kt, aDm, aF, en, g, iK, sk, bbz, ac, AH, gX, at, acJ, Pr); #c1(PLCG1) c2(NP_002651) c3(1-phosphatidylinositol 4,5-bisphosphate phosphodiesterase gamma-1 isoform a) c4(130717) c5(130718, 130719, 130720, 130721, 130722, 130723, 130724, 130725, 130726, 130727) c6( ) c7(bL, T, bB, ci, V, eA, q, ad, aT, h, bK, bq, ae, uQ, aW, ff, r, en, bw, ck, bE, Vy, wB, ei, bt); #c1(PLCG1) c2(NP_877963) c3(1-phosphatidylinositol 4,5-bisphosphate phosphodiesterase gamma-1 isoform b) c4(257042) c5(257043, 257044, 257045, 257046, 257047, 257048) c6( ) c7(bL, T, bB, ci, V, eA, q, ad, aT, h, bK, bq, ae, uQ, aW, ff, r, en, bw, ck, bE, Vy, wB, ei, bt); #c1(PLCG2) c2(NP_002652) c3(1-phosphatidylinositol 4,5-bisphosphate phosphodiesterase gamma-2) c4(130728, 130729, 130730, 130731) c5(130732, 130733, 130734, 130735, 130736, 130737, 130738, 130739, 130740) c6( ) c7(bF, crS, crT, ac, yE, aW, crU, aw, zf, jh, aT, cj, aA, h, bK, er); #c1(PLCH1) c2(NP_001124432) c3(1-phosphatidylinositol 4,5-bisphosphate phosphodiesterase eta-1 isoform a) c4(48844, 48845) c5(48846, 48847, 48848, 48849, 48850, 48851) c6( ) c7(eM, ff, en, g, bz, fq); #c1(PLCH1) c2(NP_001124433) c3(1-phosphatidylinositol 4,5-bisphosphate phosphodiesterase eta-1 isoform c) c4( ) c5(48852, 48853, 48854, 48855, 48856, 48857) c6( ) c7(eM, ff, en, g, bz, fq); #c1(PLCH1) c2(NP_055811) c3(1-phosphatidylinositol 4,5-bisphosphate phosphodiesterase eta-1 isoform b) c4(183655) c5(183656, 183657, 183658, 183659, 183660, 183661, 183662, 183663, 183664, 183665, 183666) c6( ) c7(eM, ff, en, g, bz, fq); #c1(PLCH1) c2(XP_005247296) c3(1-phosphatidylinositol 4,5-bisphosphate phosphodiesterase eta-1 isoform X4) c4(267753) c5(267754, 267755, 267756, 267757, 267758, 267759, 267760) c6( ) c7(eM, ff, en, g, bz, fq); #c1(PLCH1) c2(XP_011510866) c3(1-phosphatidylinositol 4,5-bisphosphate phosphodiesterase eta-1 isoform X2) c4( ) c5(301322, 301323) c6( ) c7(eM, ff, en, g, bz, fq); #c1(PLCL1) c2(NP_006217) c3 (inactive phospholipase C-like protein 1) c4(163006, 163007) c5(163008, 163009, 163010, 163011, 163012, 163013, 163014, 163015, 163016, 163017, 163018, 163019) c6( ) c7(bA, eR, eL, ayB, kq, ht, r, akY, dL, ah, q, v, hu, bz, af, us, ad); #c1(PLCL2) c2(NP_00137854) c3(inactive phospholipase C-like protein 2 isoform 1) c4(56028, 56029) c5(56030, 56031, 56032, 56033, 56034, 56035, 56036, 56037) c6( ) c7(y, wD, cd, bc); #c1(PLCL2) c2(NP_055999) c3(inactive phospholipase C-like protein 2 isoform 2) c4( ) c5(185041, 185042, 185043, 185044, 185045, 185046, 185047, 185048, 185049, 185050, 185051, 185052, 185053) c6( ) c7(y, wD, cd, bc); #c1(PLCL2) c2(XP_006713136) c3(inactive phospholipase C-like protein 2 isoform X1) c4(287048) c5(287049) c6( ) c7(y, wD, cd, bc); #c1(PLCXD2) c2(NP_001172035) c3(PI-PLC X domain-containing protein 2 isoform a) c4(72298) c5(72299, 72300, 72301, 72302, 72303, 72304) c6( ) c7(cF, Ea, pN); #c1(PLCXD2) c2(NP_695000) c3(PI-PLC X domain-containing protein 2 isoform b) c4(245637) c5(245638, 245639, 245640, 245641, 245642, 245643) c6( ) c7(cF, Ea, pN); #c1(PLCXD3) c2(NP_001005473) c3(PI-PLC X domain-containing protein 3) c4(21692) c5(21693, 21694, 21695, 21696, 21697, 21698, 21699, 21700, 21701, 21702) c6( ) c7(pN); #c1(PLCZ1) c2(NP_149114) c3(l-phosphatidylinositol 4,5-bisphosphate phosphodiesterase zeta-1) c4(227225, 227226, 227227) c5(227228, 227229, 227230, 227231, 227232, 227233, 227234, 227235, 227236, 227237, 227238, 227239, 227240, 227241, 227242, 227243, 227244) c6( ) c7(gU, ao); #c1(PLCZ1) c2(XP_011519185) c3(1-phosphatidylinositol 4,5-bisphosphate phosphodiesterase zeta-1 isoform X11) c4(313776, 313777) c5(313778, 313779, 313780, 313781, 313782, 313783, 313784, 313785, 313786, 313787, 313788, 313789) c6( ) c7(gU, ao); #c1(PLD1) c2(NP_001123553) c3(phospholipase Dl isoform b) c4(47475) c5(47476, 47477, 47478, 47479, 47480, 47481, 47482, 47483, 47484, 47485, 47486, 47487) c6( ) c7(bL, ai, T, wi, aW, Y, az, aT, r, aA, aF, ci, as, eA, il, fq, q, Pm); #c1(PLD1) c2(NP_002653) c3(phospholipase D1 isoform a) c4(130741) c5(130742, 130743, 130744, 130745, 130746, 130747) c6( ) c7(bL, ai, T, wi, aW, Y, az, aT, r, aA, aF, ci, as, eA, il, fq, q, Pm); #c1(PLD1) c2(XP_011511199) c3(phospholipase D1 isoform X3) c4(301673) c5(301674, 301675, 301676, 301677, 301678, 301679) c6( ) c7(bL, ai, T, wi, aW, Y, az, aT, r, aA, aF, ci, as, eA, il, fq, q, Pm); #c1(PLD1) c2(XP_011511200) c3(phospholipase D1 isoform X4) c4(301680, 301681) c5(301682, 301683, 301684, 301685, 301686, 301687) c6( ) c7(bL, ai, T, wi, aW, Y, az, aT, r, aA, aF, ci, as, eA, il, fq, q, Pm); #c1(PLD2) c2(NP_001230037) c3 (phospholipase D2 isoform PLD2B) c4(85839) c5(85840, 85841, 85842, 85843, 85844, 85845) c6( ) c7(T, dA, eR, jh, cz, il, cd, eA, q, Bc, aT, lG, hn, af, dx, d, lR, aW, r, as, beH, beJ, nM); #c1(PLD2) c2(NP_002654) c3 (phospholipase D2 isoform PLDZA) c4(130748) c5(130749, 130750, 130751, 130752, 130753, 130754) c6( ) c7(T, dA, eR, jh, cz, il, cd, eA, q, Bc, aT, lG, hn, af, dx, d, lR, aW, r, as, beH, beJ, nM); #c1(PLD3) c2(NP_001026866) c3(phospholipase D3) c4(29685, 29686, 29687, 29688) c5(29689, 29690, 29691, 29692, 29693, 29694, 29695, 29696, 29697, 29698, 29699, 29700, 29701, 29702, 29703, 29704, 29705, 29706, 29707, 29708, 29709, 29710, 29711, 29712, 29713, 29714, 29715, 29716, 29717, 29718, 29719, 29720, 29721, 29722, 29723, 29724, 29725, 29726, 29727, 29728) c6( ) c7(crW, crV, aT); #c1(PLD4) c2(NP_620145) c3(phospholipase D4) c4(237352) c5( ) c6( ) c7(y, pb); #c1(PLD5) c2(NP_001182740) c3(inactive phospholipase D5 isoform 2) c4(75217, 75218) c5(75219, 75220, 75221, 75222, 75223, 75224, 75225, 75226, 75227, 75228, 75229, 75230) c6( ) c7(V, bN); #c1(PLD5) c2(NP_689879) c3(inactive phospholipase D5 isoform 1) c4(244239) c5(244240, 244241, 244242, 244243, 244244, 244245) c6( ) c7(V, bN); #c1(PLD5) c2(XP_011542420) c3(inactive phospholipase D5 isoform X4) c4(340948) c5(340949, 340950, 340951, 340952, 340953, 340954) c6( ) c7(V, bN); #c1(PLEC) c2(NP_000436) c3(plectin isoform 1c) c4(8972, 8973) c5( ) c6( ) c7(T, arM, fR, biF, XJ, aHv, n, fd, asF, UX, crY, XM, xC, xm, biq, crX, h, K, lQ, aOh, Gc, jf, s, xz, xs, aMH, g, as, iK, dr, xA, XG, XO, arO, B, crZ); #c1(PLEC) c2(NP_958780) c3(plectin isoform If) c4(262138) c5( ) c6( ) c7(T, arM, fR, biF, XJ, aHlv, n, fd, asF, UX, crY, XM, xC, xm, biq, crX, h, K, lQ, aDh, Gc, jf, s, xz, xs, aMH, g, as, iK, dr, xA, XG, XO, arQ, B, crZ); #c1(PLEC) c2(NP_958781) c3(plectin isoform Ie) c4(262139) c5( ) c6( ) c7(T, arM, fR, biF, XJ, aHv, n, fd, asf, UX, crY, XM, xC, xm, biq, crX, h, K, lQ, aOh, Gc, jf, s, xz, xs, aMH, g, as, iK, dr, xA, XG, XO, arO, B, crZ); #c1(PLEC) c2(NP_958782) c3(plectin isoform 1) c4(262140) c5( ) c6( ) c7(T, arM, fR, biF, XJ, aHv, n, fd, asF, UX, crY, XM, xC, xm, biq, crX, h, K, lQ, aOh, Gc, jf, s, xz, xs, aMH, g, as, iK, dr, xA, XG, XO, arO, B, crZ); #c1(PLEC) c2(NP_958783) c3(plectin isoform 1d) c4(262141) c5(262142, 262143, 262144, 262145, 262146, 262147, 262148, 262149, 262150, 262151, 262152, 262153, 262154, 262155, 262156, 262157, 262158, 262159, 262160, 262161, 262162, 262163, 262164, 262165, 262166, 262167, 262168, 262169, 262170, 262171, 262172, 262173, 262174, 262175, 262176, 262177, 262178, 262179, 262180, 262181, 262182, 262183, 262184, 262185, 262186, 262187, 262188, 262189, 262190, 262191, 262192, 262193, 262194, 262195) c6( ) c7(T, arM, fR, biF, XJ, aHv, n, fd, asF, UX, crY, XM, xC, xm, biq, crX, h, K, lQ, aOh, Gc, jf, s, xz, xs, aMH, g, as, iK, dr, xA, XG, XO, arO, B, crZ); #c1(PLEC) c2(NP_958784) c3(plectin isoform Ib) c4(262196) c5( ) c6( ) c7(T, arM, fR, biF, XJ, aHv, n, fd, asF, UX, crY, XM, xC, xm, biq, crX, h, K, lQ, aOh, Gc, jf, s, xz, xs, aMH, g, as, iK, dr, xA, XG, XO, arO, B, crZ); #c1(PLEC) c2(NP_958785) c3(plectin isoform 1g) c4(262197) c5( ) c6( ) c7(T, arM, fR, biF, XJ, aHv, n, fd, asF, UX, crY, XM, xC, xm, biq, crX, h, K, lQ, aOh, Gc, jf, s, xz, xs, aMH, g, as, iK, dr, xA, XG, XO, arD, B, crZ); #c1(PLEC) c2(NP_958786) c3(plectin isoform 1a) c4(262198) c5( ) c6( ) c7(T, arM, fR, biF, XJ, aHv, n, fd, asF, UX, crY, XM, xC, xm, biq, crX, h, K, lQ, aOh, Gc, jf, s, xz, xs, aMH, g, as, iK, dr, xA, XG, XO, arO, B, crZ); #c1(PLEC) c2(XP_005251033) c3 (plectin isoform X3) c4( ) c5(270729) c6( ) c7(T, arM, fR, biF, XJ, aHv, n, fd, asF, UX, crY, XM, xC, xm, biq, crX, h, K, lQ, aDh, Gc, jf, s, xz, xs, aMH, g, as, iK, dr, xA, XG, XO, arO, B, crZ); #c1(PLEK) c2(NP_002655) c3(pleckstrin) c4(130755) c5(130756, 130757, 130758, 130759, 130760, 130761, 130762, 130763) c6( ) c7(bL, bM, T, azi, amp, v, UX, V, afU, kH, ey, bU, ahE, Um, bA, d, bT, Y, xz, fe, arK, hp, ahl, cl, iV, t, wA, asE, ck, bF, m, y, ra); #c1(PLEK2) c2(NP_057529) c3(pleckstrin-2) c4(191260) c5(191261, 19/262, 191263, 191264, 191265, 191266) c6( ) c7(Y, ad); #c1(PLEKHA1) c2(NP_001001974) c3(pleckstrin homology domain-containing family A member 1 isoform 1) c4(19237, 19238) c5(19239, 19240, 19241, 19242, 19243, 19244, 19245, 19246, 19247, 19248, 19249, 19250, 19251, 19252) c6( ) c7(Um, ZC, dD, eq, W); #c1(PLEKHA1) c2(NP_001182537) c3(pleckstrin homology domain-containing family A member 1 isoform 2) c4(75020) c5(75021, 75022, 75023, 75024, 75025, 75026, 75027, 75028, 75029, 75030, 75031) c6( ) c7(Um, ZC, dD, eq, W); #c1(PLEKHA1) c2(XP_005270077) c3(pleckstrin homology domain-containing family A member 1 isoform X5) c4(283094) c5(283095, 283096, 283097, 283098, 283099, 283100, 283101, 283102, 283103, 283104) c6( ) c7(Um, ZC, dD, eq, W); #c1(PLEKHA1) c2(XP_011538327) c3(pleckstrin homology domain-containing family A member 1 isoform X3) c4( ) c5(336025) c6( ) c7(Um, ZC, dD, eq, W); #c1(PLEKHA2) c2(NP_067636) c3(pleckstrin homology domain-containing family A member 2) c4(209119, 209120, 209121) c5(209122, 209123, 209124, 209125, 209126, 209127, 209128, 209129, 209130, 209131, 209132, 209133) c6( ) c7(jh); #c1(PLEKHA2) c2(XP_011542910) c3(pleckstrin homology domain-containing family A member 2 isoform X2) c4( ) c5(341507, 341508, 341509, 341510, 341511, 341512) c6( ) c7(jh); #c1(PLEKHA5) c2(NP_001137293) c3(pleckstrin homology domain-containing family A member 5 isoform 2) c4(55311, 55312) c5(55313, 55314, 55315, 55316, 55317, 55318, 55319, 55320, 55321, 55322) c6( ) c7(fq); #c1(PLEKHA5) c2(NP_001177789) c3(pleckstrin homology domain-containing family A member 5 isoform 3) c4( ) c5(73052, 73053, 73054, 73055, 73056, 73057) c6( ) c7(fq); #c1(PLEKHA5) c2(NP_001243399) c3(pleckstrin homology domain-containing family A member 5 isoform 4) c4(90686, 90687) c5(90688, 90689, 90690, 90691, 90692, 90693, 90694, 90695, 90696, 90697, 90698, 90699) c6( ) c7(fq); #c1(PLEKHA5) c2(NP_001243716) c3(pleckstrin homology domain-containing family A member 5 isoform 5) c4(91527) c5(91528, 91529, 91530, 91531, 91532) c6( ) c7(fq); #c1(PLEKHA5) c2(NP_061885) c3(pleckstrin homology domain-containing family A member 5 isoform 1) c4(200941, 200942, 200943, 200944) c5(200945, 200946, 200947, 200948, 200949, 200950, 200951, 200952, 200953, 200954, 200955, 200956, 200957, 200958, 200959, 200960, 200961, 200962, 200963, 200964, 200965, 200966, 200967, 200968, 200969, 200970, 200971, 200972, 200973, 200974, 200975, 200976, 200977, 200978, 200979, 200980) c6( ) c7(fq); #c1(PLEKHA5) c2(XP_005253457) c3(pleckstrin homology domain-containing family A member 5 isoform X8) c4(272496) c5(272497, 272498, 272499, 272500, 272501, 272502) c6( ) c7(fq); #c1(PLEKHA5) c2(XP_006719152) c3(pleckstrin homology domain-containing family A member 5 isoform X5) c4(292222) c5(292223, 292224, 292225, 292226, 292227, 292228, 292229) c6( ) c7(fq); #c1(PLEKHA5) c2(XP_006719159) c3(pleckstrin homology domain-containing family A member 5 isoform X2) c4(292230) c5( ) c6( ) c7(fq); #c1(PLEKHA5) c2(XP_011519018) c3(pleckstrin homology domain-containing family A member 5 isoform X6) c4(313465) c5( ) c6( ) c7(fq); #c1(PLEKHA6) c2(NP_055750) c3(pleckstrin homology domain-containing family A member 6) c4(183277) c5(183278, 183279, 183280, 183281, 183282, 183283) c6( ) c7(cz); #c1(PLEKHA6) c2(XP_005245024) c3(pleckstrin homology domain-containing family A member 6 isoform X15) c4(266220) c5(266221, 266222, 266223, 266224, 266225, 266226) c6( ) c7(cz); #c1(PLEKHA6) c2(XP_006711275) c3(pleckstrin homology domain-containing family A member 6 isoform X9) c4(285839) c5( ) c6( ) c7(cz); #c1(PLEKHA6) c2(XP_006711276) c3(pleckstrin homology domain-containing family A member 6 isoform X3) c4(285840, 285841) c5(285842, 285843, 285844, 285845, 285846, 285847, 285848, 285849, 285850, 285851, 285852, 285853) c6( ) c7(cz); #c1(PLEKHA6) c2(XP_006711278) c3(pleckstrin homology domain-containing family A member 6 isoform X7) c4(285854) c5(285855, 285856, 285857, 285858, 285859, 285860) c6( ) c7(cz); #c1(PLEKHA6) c2(XP_006711281) c3(pleckstrin homology domain-containing family A member 6 isoform X11) c4(285861) c5(285862, 285863, 285864, 285865, 285866, 285867) c6( ) c7(cz); #c1(PLEKHA6) c2(XP_011507598) c3(pleckstrin homology domain-containing family A member 6 isoform X6) c4(297511) c5(297512, 297513, 297514, 297515, 297516, 297517) c6( ) c7(cz); #c1(PLEKHA6) c2(XP_011507608) c3(pleckstrin homology domain-containing family A member 6 isoform X19) c4( ) c5(297518, 297519, 297520, 297521, 297522, 297523) c6( ) c7(cz); #c1(PLEKHA7) c2(NP_778228) c3(pleckstrin homology domain-containing family A member 7) c4(251428) c5(251429, 251430, 251431, 251432, 251433, 251434, 251435, 251436, 251437, 251438, 251439, 251440) c6( ) c7(oe, sa, cz, in, dw, AO, lJ, ad); #c1(PLEKHA7) c2(XP_011518216) c3(pleckstrin homology domain-containing family A member 7 isoform X6) c4(312253) c5( ) c6( ) c7(oe, sa, cz, in, dw, AQ, lJ, ad); #c1(PLEKHA7) c2(XP_011518218) c3(pleckstrin homology domain-containing family A member 7 isoform X8) c4(312254) c5(312255, 312256, 312257, 312258, 312259, 312260) c6( ) c7(oe, sa, cz, in, dw, AO, lJ, ad); #c1(PLEKHB1) c2(NP_001123505) c3(pleckstrin homology domain-containing family B member 1 isoform c) c4(47210, 47211, 47212, 47213, 47214, 47215) c5(47216, 47217, 47218, 47219, 47220, 47221, 47222, 47223, 47224, 47225, 47226) c6( ) c7(aW, W, d, K, aT); #c1(PLEKHB1) c2(NP_001123506) c3(pleckstrin homology domain-containing family B member 1 isoform b) c4( ) c5(47227, 47228, 47229, 47230, 47231, 47232, 47233, 47234, 47235, 47236) c6( ) c7(aW, W, d, K, aT); #c1(PLEKHB1) c2(NP_001123508) c3(pleckstrin homology domain-containing family B member 1 isoform d) c4( ) c5(47237, 47238, 47239, 47240, 47241, 47242, 47243, 47244, 47245, 47246, 47247, 47248, 47249, 47250, 47251, 47252, 47253, 47254, 47255, 47256, 47257, 47258, 47259) c6( ) c7(aW, W, d, K, aT); #c1(PLEKHB1) c2(NP_067023) c3(pleckstrin homology domain-containing family B member 1 isoform a) c4(208466, 208467, 208468, 208469) c5(208470, 208471, 208472, 208473, 208474, 208475, 208476, 208477) c6( ) c7(aW, W, d, K, aT); #c1(PLEKHB1) c2(XP_01543496) c3(pleckstrin homology domain-containing family B member 1 isoform X3) c4( ) c5(342371, 342372, 342373, 342374, 342375, 342376, 342377, 342378, 342379, 342380, 342381, 342382) c6( ) c7(aW, W, d, K, aT); #c1(PLEKHD1) c2(NP_001154970) c3(pleckstrin homology domain-containing family D member 1) c4(61241) c5(61242, 61243, 61244, 61245, 61246, 61247) c6( ) c7(aw); #c1(PLEKHF1) c2(NP_077286) c3(pleckstrin homology domain-containing family F member 1) c4(214942) c5(214943, 214944, 214945, 214946, 214947) c6( ) c7(T, csc, csb, lo, dL, t, csa); #c1(PLEKHF1) c2(XP_005259313) c3 (pleckstrin homology domain-containing family F member 1 isoform X2) c4( ) c5(276060) c6( ) c7(T, csc, csb, lo, dL, t, csa); #c1(PLEKHF2) c2(NP_078889) c3(pleckstrin homology domain-containing family F member 2) c4(215954) c5(215955, 215956, 215957, 215958, 215959, 215960, 215961, 215962, 215963, 215964) c6( ) c7(ey); #c1(PLEKHG1) c2(NP_001025055) c3(pleckstrin homology domain-containing family G member 1) c4( ) c5(29274, 29275, 29276, 29277, 29278, 29279, 29280, 29281, 29282, 29283, 29284, 29285, 29286, 29287, 29288, 29289, 29290, 29291) c6( ) c7(ww, j, bA); #c1(PLEKHG1) c2(XP_011534283) c3(pleckstrin homology domain-containing family G member 1 isoform X2) c4(330653, 330654, 330655) c5(330656, 330657, 330658) c6( ) c7(ww, j, bA); #c1(PLEKHG1) c2(XP_011534284) c3(pleckstrin homology domain-containing family G member 1 isoform X4) c4( ) c5(330659) c6( ) c7(ww, j, bA); #c1(PLEKHG2) c2(NP_073746) c3(pleckstrin homology domain-containing family G member 2) c4(213440, 213441, 213442) c5(213443, 213444, 213445, 213446, 213447, 213448, 213449, 213450, 213451, 213452, 213453, 213454, 213455, 213456, 213457, 213458) c6( ) c7(av, iT, ky); #c1(PLEKHG2) c2(XP_006723397) c3(pleckstrin homology domain-containing family G member 2 isoform X5) c4(295187) c5(295188, 295189, 295190, 295191, 295192, 295193, 295194, 295195, 295196, 295197) c6( ) c7(av, iT, ky); #c1(PLEKHG2) c2(XP_011525536) c3(pleckstrin homology domain-containing family G member 2 isoform X4) c4(319956) c5(319957, 319958, 319959, 319960, 319961, 319962, 319963, 319964, 319965, 319966, 319967, 319968) c6( ) c7(av, iT, ky); #c1(PLEKHG3) c2(XP_011534927) c3(pleckstrin homology domain-containing family G member 3 isoform X2) c4(331507, 331508) c5( ) c6( ) c7(bN); #c1(PLEKHG3) c2(XP_011534929) c3(pleckstrin homology domain-containing family G member 3 isoform X4) c4( ) c5(331509, 331510, 331511) c6( ) c7(bN); #c1(PLEKHG4) c2(NP_001123201) c3(puratrophin-1 isoform 1) c4(46811, 46812) c5(46813, 46814, 46815, 46816, 46817, 46818, 46819, 46820, 46821, 46822, 46823, 46824, 46825, 46826, 46827) c6( ) c7(Ng, KB, ky, kB, Kv, rf, yZ, bX, aqO); #c1(PLEKHG4) c2(NP_001123203) c3(puratrophin-1 isoform 2) c4(46828) c5(46829, 46830, 46831, 46832, 46833, 46834, 46835) c6( ) c7(Ng, KB, ky, kB, Kv, rf, yZ, bX, aqO); #c1(PLEKHG4) c2(XP_011521291) c3(puratrophin-1 isoform X2) c4( ) c5(316183, 316184, 316185, 316186, 316187) c6( ) c7(Ng, KB, ky, kB, Kv, rf, yZ, bX, aqO); #c1(PLEKHG6) c2(NP_001138328) c3(pleckstrin homology domain-containing family G member 6 isoform a) c4(56157) c5(56158, 56159, 56160, 56161, 56162, 56163, 56164, 56165, 56166, 56167, 56168, 56169) c6( ) c7(aW, aT); #c1(PLEKHG6) c2(NP_001138329) c3(pleckstrin homology domain-containing family G member 6 isoform b) c4(56170, 56171) c5(56172, 56173, 56174, 56175, 56176, 56177, 56178, 56179, 56180, 56181, 56182, 56183, 56184) c6( ) c7(aW, aT); #c1(PLEKHH2) c2(NP_742066) c3(pleckstrin homology domain-containing family H member 2) c4(247932) c5(247933, 247934, 247935, 247936, 247937, 247938) c6( ) c7(vw); #c1(PLEKHM1) c2(NP_055613) c3(pleckstrin homology domain-containing family M member 1) c4(182282) c5(182283, 182284, 182285, 182286, 182287, 182288, 182289, 182290, 182291, 182292, 182293) c6( ) c7(zs, Hz, m, csd, an, cse); #c1(PLEKHM1) c2(XP_006722265) c3(pleckstrin homology domain-containing family M member 1 isoform X4) c4( ) c5(294674) c6( ) c7(zs, Hz, m, csd, an, cse); #c1(PLEKHM1) c2(XP_011523827) c3(pleckstrin homology domain-containing family M member 1 isoform X2) c4(318355, 318356, 318357) c5(318358, 318359, 318360, 318361, 318362, 318363, 318364, 318365, 318366, 318367, 318368, 318369, 318370, 318371, 318372, 318373, 318374, 318375, 318376, 318377, 318378, 318379, 318380) c6( ) c7(zs, Hz, m, csd, an, cse); #c1(PLEKHM1) c2(XP_011523828) c3(pleckstrin homology domain-containing family M member 1 isoform X3) c4(318381, 318382, 318383, 318384) c5(318385, 318386, 318387, 318388, 318389, 318390, 318391, 318392, 318393, 318394, 318395, 318396, 318397, 318398, 318399, 318400, 318401, 318402, 318403, 318404, 318405, 318406) c6( ) c7(zs, Hz, m, csd, an, cse); #c1(PLEKHO1) c2(NP_001291653) c3(pleckstrin homology domain-containing family 0 member 1 isoform c) c4( ) c5(116300, 116301, 116302, 116303, 116304) c6( ) c7(eF, eH); #c1(PLEKHO2) c2(NP_079477) c3(pleckstrin homology domain-containing family 0 member 2 isoform 1) c4(218237, 218238) c5(218239, 218240, 218241, 218242, 218243, 218244, 218245, 218246, 218247, 218248, 218249, 218250) c6( ) c7(j); #c1(PLG) c2(NP_000292) c3(plasminogen isoform 1 precursor) c4(6496, 6497) c5(6498, 6499, 6500, 6501, 6502, 6503, 6504, 6505, 6506, 6507, 6508, 6509, 6510, 6511, 6512, 6513, 6514, 6515, 6516, 6517, 6518, 6519, 6520, 6521, 6522, 6523, 6524, 6525, 6526, 6527, 6528, 6529, 6530, 6531, 6532, 6533, 6534, 6535, 6536, 6537, 6538, 6539, 6540, 6541, 6542, 6543, 6544, 6545, 6546, 6547, 6548, 6549, 6550, 6551, 6552, 6553, 6554, 6555, 6556, 6557, 6558, 6559, 6560, 6561, 6562, 6563, 6564, 6565, 6566, 6567, 6568, 6569, 6570, 6571, 6572, 6573, 6574, 6575, 6576, 6577, 6578, 6579, 6580, 6581, 6582, 6583, 6584, 6585, 6586, 6587, 6588, 6589, 6590, 6591, 6592, 6593, 6594, 6595, 6596, 6597, 6598, 6599, 6600, 6601, 6602, 6603, 6604, 6605, 6606, 6607, 6608, 6609, 6610, 6611, 6612, 6613, 6614, 6615, 6616, 6617, 6618, 6619, 6620, 6621, 6622, 6623, 6624, 6625, 6626, 6627, 6628, 6629, 6630, 6631, 6632, 6633, 6634, 6635, 6636, 6637, 6638, 6639, 6640, 6641, 6642, 6643, 6644, 6645, 6646, 6647, 6648, 6649) c6(6650) c7(DS, oO, T, bM, lD, aC, azf, xS, ea, if, e, al, Xi, l, eb, bB, aiC, ame, ci, bRn, ss, ip, bv, azY, dY, aE, M, K, hu, blx, dQ, eL, cp, bzH, hm, ly, eG, aW, az, YG, pD, aUG, zC, g, as, eF, axp, aVc, DL, bwe, aUF, qJ, eM, iT, mx, vy, wx, zv, Hb, m, fy, bcD, dD, wu, wJ, aw, ht, NT, ez, n, aj, blz, fr, ct, aBl, qN, cd, ai, El, aDF, ft, ju, rC, cU, nm, jH, dL, Gc, wC, OV, csj, csi, sq, EU, fQ, aq, fe, aZf, lx, kT, ok, W, dB, se, ayS, RJ, mg, hL, qq, uz, at, bm, eo, qF, it, B, bL, jv, dA, mr, Dh, dK, fG, cbc, aA, cz, v, j, C, nS, V, q, J, Yh, eA, eJ, MR, aHb, aT, cy, jj, an, rz, ae, af, aWK, kl, bf, wr, Y, Pg, aLJ, r, en, cl, wA, t, sS, nM, Dy, ac, lV, hc, dh, wR, dy, fD, csf, ao, hl, aCA, id, qb, iW, aET, bS, dG, ah, eq, ad, BY, csg, cit, dP, hd, hX, av, sN, Ot, ds, aQ, wm, jo, aGh, lz, vl, uX, d, lU, ue, cW, csk, ff, eH, aP, cr, eZ, dN, gl, afi, gE, aF, Z, csh, bz, dr, qa, ek, LB, bF, DT, zr, bE, ag, dX, aS, XO, be, ei, y, X, nQ, bpw); #c1(PLG) c2(NP_001161810) c3 (plasminogen isoform 2 precursor) c4(66050) c5(66051, 66052, 66053, 66054, 66055, 66056, 66057, 66058, 66059, 66060) c6( ) c7(DS, oO, T, bM, lD, aC, azf, xS, ea, if, e, al, Xi, l, eb, bB, aiC, ame, ci, bRn, ss, ip, bv, azY, dY, aE, M, K, hu, blx, dQ, eL, cp, bzH, hm, ly, eG, aW, az, YG, pD, aUG, zC, g, as, eF, axp, aVc, DL, bwe, aUF, qJ, eM, iT, mx, vy, wx, zv, Hb, m, fy, bcO, dD, wu, wJ, aw, ht, NT, ez, n, aj, blz, fr, ct, aBl, qN, cd, ai, El, aDF, ft, ju, rC, cU, nm, jH, dL, Gc, wC, OV, csj, csi, sq, EU, fQ, aq, fe, aZf, lx, KT, ok, W, dB, se, ayS, RJ, mg, hL, qq, uz, at, bm, eo, qF, it, B, bL, jv, dA, mr, Dh, dK, fG, cbc, aA, cz, v, j, C, nS, V, q, J, Yh, eA, eJ, MR, aHb, aT, cy, jj, an, rz, ae, af, aWK, kl, bf, wr, Y, Pg, aLJ, r, en, cl, wA, t, sS, nM, Dy, ac, lV, hc, dh, wR, dy, fD, csf, ao, hl, aCA, id, qb, iW, aET, bS, dG, ah, eq, ad, BY, csg, cit, dP, hd, hX, av, sN, Ot, ds, aQ, wm, jo, aGh, lz, vl, uX, d, lU, ue, cW, csk, ff, eH, aP, cr, eZ, dN, gl, afi, gE, aF, Z, csh, bz, dr, qa, ek, LB, bF, DT, zr, bE, ag, dX, aS, XO, be, ei, y, X, nQ, bpw); #c1(PLGLB2) c2(NP_002656) c3(plasminogen-like protein B precursor) c4(130764) c5(130765, 130766, 130767, 130768, 130769, 130770, 130771, 130772, 130773, 130774, 130775, 130776, 130777, 130778, 130779, 130780, 130781, 130782, 130783, 130784, 130785, 130786, 130787) c6(130788, 130789, 130790, 130791, 130792) c7(j); #c1(PLIN1) c2(XP_005254991) c3(perilipin-1 isoform X1) c4(273470, 273471) c5(273472, 273473, 273474, 273475, 273476, 273477, 273478, 273479, 273480, 273481, 273482) c6( ) c7(bL, kl, LL, ac, wr, aZ, QJ, axd, fc, M, cz, jy, mf, v, ei, csl, eu, Ql); #c1(PLIN2) c2(NP_001113) c3(perilipin-2) c4(43326, 43327, 43328, 43329, 43330) c5(43331, 43332, 43333, 43334, 43335, 43336, 43337, 43338, 43339, 43340, 43341, 43342, 43343, 43344, 43345, 43346, 43347, 43348, 43349, 43350, 43351, 43352, 43353, 43354, 43355, 43356, 43357, 43358, 43359, 43360) c6( ) c7(T, nl, axd, aA, v, zx, ci, fr, avD, py, aew, nJ, xX, iU, fc, mo, nC, amS, W, iA, go, cS, boH); #c1(PLIN3) c2(NP_001157666) c3(perilipin-3 isoform 3) c4(62395) c5(62396, 62397, 62398, 62399, 62400, 62401) c6( ) c7(v, K, vi); #c1(PLIN3) c2(NP_005808) c3(perilipin-3 isoform 1) c4(159617) c5(159618, 159619, 159620, 159621, 159622, 159623) c6( ) c7(v, K, vi); #c1(PLIN4) c2(NP_001073869) c3(perilipin-4) c4(36207) c5(36208, 36209, 36210, 36211, 36212, 36213) c6( ) c7(om, v, bA); #c1(PLIN5) c2(NP_001013728) c3(perilipin-5) c4(25737) c5(25738, 25739, 25740, 25741, 25742, 25743) c6( ) c7(v); #c1(PLK1) c2(NP_005021) c3(serine/threonine-protein kinase PLK1) c4(152244, 152245, 152246) c5(152247, 152248, 152249, 152250, 152251, 152252, 152253, 152254, 152255, 152256, 152257, 152258, 152259, 152260, 152261, 152262, 152263, 152264) c6( ) c7(T, aC, pf, fd, bB, ci, x, bv, aE, ja, K, hu, MO, pk, eL, Jl, aW, g, eF, cm, eM, nB, iA, iT, m, k, Or, ht, NT, n, fr, qN, ft, no, eV, dL, aq, QK, yR, iO, bP, asl, dB, hR, at, it, B, aMk, fM, eX, aA, hy, q, eA, eJ, aT, an, ae, ne, jf, iU, nO, oV, Y, ev, r, en, cl, t, ck, kP, au, bt, hS, id, jh, KO, ad, dP, hd, Db, d, BN, ff, eH, aF, iR, bz, es, bE, adg, X); #c1(PLK2) c2(NP_001239155) c3(serine/threonine-protein kinase PLK2 isoform 2) c4(88349) c5(88350, 88351, 88352, 88353, 88354, 88355) c6( ) c7(T, fV, jh, fd, ci, zP, XX, av, Bl, aE, aT, dl, K, pk, eL, af, ak, zQ, d, ff, eH, aq, en, g, eF, t, iR, m, bt, B); #c1(PLK2) c2(NP_006613) c3(serine/threonine-protein kinase PLK2 isoform 1) c4(166638, 166639) c5(166640, 166641, 166642, 166643, 166644, 166645, 166646, 166647, 166648, 166649, 166650, 166651) c6( ) c7(T, fV, jh, fd, ci, zP, XX, av, Bl, aE, aT, dL, K, pk, eL, af, ak, zQ, d, ff, eH, aq, en, g, eF, t, iR, m, bt, B); #c1(PLK4) c2(NP_001177728) c3(serine/threonine-protein kinase PLK4 isoform 2) c4(72935) c5(72936, 72937, 72938, 72939, 72940, 72941) c6( ) c7(T, yi, B, aT, r, aSW, kv, K, Nj, QK, ae, q, ad); #c1(PLK4) c2(NP_001177730) c3(serine/threonine-protein kinase PLK4 isoform 3) c4( ) c5(72942, 72943, 72944, 72945, 72946) c6( ) c7(T, yi, B, aT, r, aSW, kv, K, Nj, QK, ae, q, ad); #c1(PLK4) c2(NP_055079) c3(serine/threonine-protein kinase PLK4 isoform 1) c4(178837, 178838) c5(178839, 178840, 178841, 178842, 178843, 178844, 178845) c6( ) c7(T, yi, B, aT, r, aSW, kv, K, Nj, QK, ae, q, ad); #c1(PLK5) c2(NP_001230008) c3(inactive serine/threonine-protein kinase PLK5) c4(85773) c5(85774, 85775, 85776, 85777, 85778, 85779) c6( ) c7(dB, d, as); #c1(PLLP) c2(NP_057077) c3(plasmolipin) c4(188822) c5(188823, 188824, 188825, 188826, 188827, 188828) c6( ) c7(agU, af, Tp); #c1(PLN) c2(NP_002658) c3(cardiac phospholamban) c4(130793) c5(130794, 130795, 130796, 130797, 130798, 130799, 130800) c6( ) c7(bL, T, mr, acl, e, aw, cz, akA, sl, cd, csm, cc, cq, Bc, JD, Ml, h, vc, mu, anR, csn, mo, ck, akF, iT, wK, eB, mx, ll, gZ, eo, bD, bt); #c1(PLOD2) c2(NP_000926) c3(procollagen-lysine,2-oxoglutarate 5-dioxygenase 2 isoform 2 precursor) c4(17393) c5(17394, 17395, 17396, 17397, 17398, 17399, 17400, 17401) c6( ) c7(iU, aW, aRV, yg, aT, buA, uo, uJ, aq, ey, zp, lR, pb, cso, gT, Eh, B, t); #c1(PLOD2) c2(NP_891988) c3(procollagen-lysine,2-oxoglutarate 5-dioxygenase 2 isoform 1 precursor) c4(257397, 257398, 257399) c5(257400, 257401, 257402, 257403, 257404, 257405, 257406, 257407, 257408, 257409, 257410, 257411, 257412, 257413, 257414, 257415) c6( ) c7(iU, aW, aRV, yg, aT, buA, uo, uJ, aq, ey, zp, lR, pb, cso, gT, Eh, B, t); #c1(PLOD2) c2(XP_005247592) c3 (procollagen-lysine,2-oxoglutarate 5-dioxygenase 2 isoform X1) c4( ) c5(267874, 267875, 267876, 267877, 267878, 267879, 267880, 267881) c6( ) c7(iU, aW, aRV, yg, aT, buA, uo, uJ, aq, ey, zp, lR, pb, cso, gT, Eh, B, t); #c1(PLP1) c2(NP_000524) c3(myelin proteolipid protein isoform 1) c4(11810, 11811, 11812, 11813, 11814, 11815) c5(11816, 11817, 11818, 11819, 11820, 11821, 11822, 11823, 11824, 11825, 11826, 11827, 11828, 11829, 11830, 11831, 11832, 11833, 11834, 11835, 11836, 11837, 11838, 11839, 11840, 11841, 11842, 11843, 11844, 11845, 11846, 11847, 11848, 11849, 11850, 11851, 11852, 11853, 11854, 11855) c6( ) c7(ach, eR, dM, agU, sM, oR, GC, csq, fu, bX, mF, V, aR, ke, aV, lu, K, cb, Jj, aHU, as, dr, bN, asE, On, cpE, ahN, KE, TJ, WE, aGY, U, oh, Lq, Da, bk, bee, csp); #c1(PLP1) c2(NP_955772) c3(myelin proteolipid protein isoform 2) c4(261946, 261947, 261948) c5(261949, 261950, 261951, 261952, 261953, 261954, 261955, 261956, 261957, 261958, 261959, 261960, 261961, 261962, 261963, 261964, 261965, 261966) c6( ) c7(ach, eR, dM, agU, sM, oR, GC, csq, fu, bX, mF, V, aR, ke, aV, lu, K, cb, Jj, aHU, as, dr, bN, asE, On, cpE, ahN, KE, TJ, wE, aGY, U, oh, Lq, Da, bk, bee, csp); #c1(PLP2) c2(NP_002659) c3(proteolipid protein 2) c4(130801, 130802) c5(130803, 130804, 130805, 130806, 130807, 130808, 130809, 130810, 130811, 130812, 130813, 130814) c6( ) c7(nf, B, K); #c1(PLRG1) c2(NP_001188493) c3(pleiotropic regulator 1 isoform 2) c4(79218) c5(79219, 79220, 79221, 79222, 79223, 79224) c6( ) c7(By, T, bz, it); #c1(PLRG1) c2(NP_002660) c3 (pleiotropic regulator 1 isoform 1) c4(130815, 130816) c5(130817, 130818, 130819, 130820, 130821, 130822, 130823, 130824, 130825, 130826, 130827, 130828, 130829) c6( ) c7(By, T, bz, it); #c1(PLS1) c2(NP_001138791) c3(plastin-1) c4(57698, 57699, 57700, 57701, 57702, 57703, 57704) c5(57705, 57706, 57707, 57708, 57709, 57710, 57711, 57712, 57713, 57714, 57715, 57716, 57717, 57718, 57719, 57720, 57721, 57722, 57723, 57724, 57725, 57726, 57727, 57728, 57729, 57730, 57731, 57732, 57733, 57734, 57735, 57736, 57737, 57738, 57739, 57740, 57741, 57742, 57743, 57744, 57745, 57746, 57747, 57748, 57749, 57750, 57751, 57752, 57753, 57754) c6( ) c7(bL, iu, Ol, ds, axW, aq, zo); #c1(PLS3) c2(NP_001269266) c3(plastin-3 isoform 3) c4(101001, 101002) c5(101003, 101004, 101005, 101006, 101007, 101008, 101009, 101010, 101011, 101012, 101013, 101014) c6( ) c7(eU, bE, r, kf, n, bB, zp, q, bA); #c1(PLS3) c2(NP_001269267) c3 (plastin-3 isoform 4) c4( ) c5(101015, 101016, 101017, 101018, 101019, 101020, 101021, 101022, 101023, 101024, 101025) c6( ) c7(eU, bE, r, kf, n, bB, zp, q, bA); #c1(PLS3) c2(XP_011535836) c3(plastin-3 isoform X1) c4(333047, 333048, 333049) c5(333050, 333051, 333052, 333053, 333054, 333055, 333056, 333057, 333058, 333059, 333060, 333061, 333062, 333063) c6( ) c7(eU, bE, r, kf, n, bB, zp, q, bA); #c1(PLSCR1) c2(NP_066928) c3(phospholipid scramblase 1) c4(207530, 207531, 207532) c5(207533, 207534, 207535, 207536, 207537, 207538, 207539, 207540, 207541, 207542, 207543, 207544) c6( ) c7(ck, iT, ds, bud, ag, eZ, ci, m, jj, t, bt, kS, ek); #c1(PLSCR1) c2(XP_011511209) c3(phospholipid scramblase 1 isoform X3) c4( ) c5(301688, 301689, 301690, 301691, 301692, 301693) c6( ) c7(ck, iT, ds, bud, ag, eZ, ci, m, jj, t, bt, kS, ek); #c1(PLSCR3) c2(NP_065093) c3(phospholipid scramblase 3) c4(203838, 203839) c5(203840, 203841, 203842, 203843, 203844, 203845, 203846, 203847, 203848, 203849, 203850, 203851, 203852, 203853, 203854, 203855, 203856, 203857, 203858, 203859) c6( ) c7(cm, v); #c1(PLSCR4) c2(NP_001121778) c3(phospholipid scramblase 4 isoform b) c4(46129, 46130) c5(46131, 46132, 46133, 46134, 46135, 46136, 46137, 46138, 46139, 46140, 46141, 46142, 46143) c6( ) c7(Bl, ft, eD, jh, rO, bS, cd); #c1(PLSCR4) c2(NP_001170775) c3(phospholipid scramblase 4 isoform c) c4( ) c5(69507, 69508, 69509, 69510, 69511) c6( ) c7(Bl, ft, eD, jh, rO, bS, cd); #c1(PLSCR4) c2(NP_065086) c3(phospholipid scramblase 4 isoform a) c4(203794, 203795, 203796, 203797, 203798) c5(203799, 203800, 203801, 203802, 203803, 203804, 203805, 203806, 203807, 203808, 203809, 203810, 203811, 203812, 203813, 203814, 203815, 203816, 203817, 203818, 203819, 203820, 203821, 203822, 203823, 203824, 203825, 203826, 203827, 203828, 203829, 203830) c6( ) c7(Bl, ft, eD, jh, rD, bS, cd); #c1(PLSCR5) c2(NP_001078889) c3(phospholipid scramblase family member 5) c4(37955) c5(37956, 37957, 37958, 37959, 37960, 37961, 37962, 37963, 37964, 37965) c6( ) c7(cd); #c1(PLTP) c2(NP_001229849) c3(phospholipid transfer protein isoform c precursor) c4(85409) c5(85410, 85411, 85412, 85413, 85414, 85415) c6( ) c7(bL, dD, ea, e, aj, v, j, eq, J, Zu, dY, nX, aT, K, dQ, qO, af, cW, si, ZB, mg, vQ); #c1(PLTP) c2(NP_001229850) c3 (phospholipid transfer protein isoform d) c4( ) c5(85416, 85417, 85418, 85419, 85420) c6( ) c7(bL, dD, ea, e, aj, v, j, eq, J, Zu, dY, nX, aT, K, dQ, qO, af, cW, si, ZB, mg, vQ); #c1(PLTP) c2(NP_006218) c3(phospholipid transfer protein isoform a precursor) c4(163020, 163021, 163022) c5(163023, 163024, 163025, 163026, 163027, 163028, 163029, 163030, 163031, 163032, 163033, 163034, 163035, 163036, 163037, 163038) c6( ) c7(bL, dD, ea, e, aj, v, j, eq, J, Zu, dY, nX, aT, K, dQ, qO, af, cW, si, ZB, mg, vQ); #c1(PLTP) c2(NP_872617) c3(phospholipid transfer protein isoform b precursor) c4(256532) c5(256533, 256534, 256535, 256536, 256537, 256538) c6( ) c7(bL, dD, ea, e, aj, v, j, eq, J, Zu, dY, nX, aT, K, dl, qO, af, cW, si, ZB, mg, vQ); #c1(PLVAP) c2(NP_112600) c3(plasmalemma vesicle-associated protein) c4(221533) c5(221534, 221535, 221536, 221537, 221538, 221539) c6( ) c7(dB, WS, je, iM); #c1(PLXDC1) c2(NP_065138) c3(plexin domain-containing protein 1 precursor) c4(204153, 204154, 204155, 204156, 204157, 204158, 204159, 204160) c5(204161, 204162, 204163, 204164, 204165, 204166, 204167, 204168, 204169, 204/70, 204171, 204172, 204173, 204174, 204/75, 204/76, 204177, 204178, 204179, 204180, 204181, 204182, 204183, 204184, 204185, 204186, 204187, 204188, 204189, 204190, 204191, 204192, 204193, 204194, 204195, 204196, 204197, 204198, 204199, 204200, 204201, 204202, 204203, 204204, 204205, 204206, 204207, 204208, 204209, 204210, 204211, 204212, 204213, 204214, 204215, 204216, 204217, 204218, 204219, 204220, 204221, 204222) c6( ) c7(eF, vx, T, vy); #c1(PLXDC2) c2(NP_001269665) c3(plexin domain-containing protein 2 isoform 2) c4(102467) c5(102468, 102469, 102470, 102471, 102472, 102473) c6( ) c7(pZ, vy, gZ, aj, aS, j, bX, fq, cd, J); #c1(PLXDC2) c2(NP_116201) c3(plexin domain-containing protein 2 isoform 1 precursor) c4(225724, 225725, 225726) c5(225727, 225728, 225729, 225730, 225731, 225732, 225733, 225734, 225735, 225736, 225737, 225738, 225739, 225740, 225741, 225742, 225743, 225744) c6( ) c7(pZ, vy, gZ, aj, aS, j, bX, fq, cd, J); #c1(PLXNA1) c2(NP_115618) c3(plexin-A1 precursor) c4(223375) c5(223376, 223377, 223378, 223379, 223380, 223381, 223382, 223383, 223384, 223385, 223386) c6( ) c7(T, ib, aE, nO, aW, bOL, eL, aT, KO, dL, m, ci, er, ae, eA, ad, hS); #c1(PLXNA1) c2(XP_011511211) c3 (plexin-A1 isoform X1) c4(301694) c5( ) c6( ) c7(T, ib, aE, nO, aW, bOL, eL, aT, KO, dL, m, ci, er, ae, eA, ad, hS); #c1(PLXNA2) c2(NP_079455) c3(plexin-A2 precursor) c4(218172) c5(218173, 218174, 218175, 218176, 218177, 218178) c6( ) c7(bGg, bL, fM, cdY, aZ, iW, oe, nx, e, aw, jh, To, cor, jA, ci, nA, csr, Jt, ad, aCJ, bxi, an, he, aQu, db, ae, bA, d, cm, Uk, hq, agS, ck, KE, W, bnP, alx, vi); #c1(PLXNA2) c2(XP_005273222) c3(plexin-A2 isoform X3) c4( ) c5(284370, 284371) c6( ) c7(bGg, bL, fM, cdY, aZ, iW, oe, nx, e, aw, jh, To, cor, jA, ci, nA, csr, Jt, ad, aCJ, bxi, an, he, aQu, db, ae, bA, d, cm, Uk, hq, agS, ck, KE, W, bnP, aQx, vi); #c1(PLXNA3) c2(NP_059984) c3(plexin-A3 precursor) c4(193477) c5(193478, 193479, 193480, 193481, 193482, 193483) c6( ) c7(xS, Fn, cA, ajv, hL, cz, bO, eZ, afE, hq); #c1(PLXNA4) c2(NP_001099013) c3(plexin-A4 isoform 3 precursor) c4(41655) c5(41656, 41657, 41658, 41659, 41660, 41661) c6( ) c7(bM, dY, Y, oe, U, eZ); #c1(PLXNA4) c2(XP_005250743) c3(plexin-A4 isoform X2) c4(270500) c5(270501, 270502, 270503, 270504, 270505, 270506, 270507, 270508, 270509, 270510) c6( ) c7(bM, dY, Y, oe, U, eZ); #c1(PLXNA4) c2(XP_005250744) c3(plexin-A4 isoform X1) c4(270511) c5(270512, 270513, 270514, 270515, 270516, 270517) c6( ) c7(bM, dY, Y, oe, U, eZ); #c1(PLXNB1) c2(NP_001123554) c3 (plexin-B1 precursor) c4(47488, 47489, 47490) c5(47491, 47492, 47493, 47494, 47495, 47496, 47497, 47498, 47499, 47500, 47501, 47502, 47503, 47504, 47505, 47506, 47507, 47508, 47509, 47510, 47511, 47512, 47513, 47514, 47515, 47516, 47517, 47518, 47519, 47520, 47521, 47522, 47523, 47524, 47525, 47526) c6(47527, 47528, 47529, 47530, 47531, 47532, 47533, 47534, 47535, 47536) c7(T, sw, Y, aW, if, aT, cj, n, aj, fr, ci, m, qN, g, t, agT, J); #c1(PLXNB3) c2(NP_001156729) c3(plexin-83 isoform 2) c4(61843, 61844, 61845, 61846) c5(61847, 61848, 61849, 61850, 61851, 61852, 61853, 61854, 61855, 61856, 61857, 61858, 61859, 61860, 61861, 61862, 61863, 61864) c6( ) c7(T, eA); #c1(PLXNB3) c2(NP_005384) c3(plexin-B3 isoform 1 precursor) c4(155596, 155597, 155598, 155599) c5(155600, 155601, 155602, 155603, 155604, 155605, 155606, 155607, 155608, 155609, 155610, 155611, 155612, 155613, 155614, 155615, 155616, 155617, 155618, 155619, 155620, 155621, 155622, 155623, 155624, 155625, 155626, 155627, 155628, 155629) c6( ) c7(T, eA); #c1(PLXNC1) c2(NP_005752) c3 (plexin-Cl precursor) c4(159011, 159012, 159013) c5(159014, 159015, 159016, 159017, 159018, 159019, 159020, 159021, 159022, 159023, 159024, 159025) c6( ) c7(dP, T, Y, cp, an); #c1(PLXND1) c2(NP_055918) c3(plexin-Di precursor) c4(184474, 184475) c5(184476, 184477, 184478, 184479, 184480, 184481) c6( ) c7(T, anB, dB, Y, RX, aA, cY); #c1(PLXND1) c2(XP_011510890) c3(plexin-D1 isoform X1) c4( ) c5(301357, 301358, 301359, 301360, 301361, 301362) c6( ) c7(T, anB, dB, Y, RX, aA, cY); #c1(PM20D1) c2(NP_689704) c3(probable carboxypeptidase PM20D1 precursor) c4(243624) c5(243625, 243626, 243627, 243628, 243629, 243630, 243631, 243632) c6(243633, 243634) c7(aiV, wz, aq, afD, an, iR, B); #c1(PMAIP1) c2(NP_066950) c3(phorbol-12-myristate-13-acetate-induced protein 1) c4(207658, 207659) c5(207660, 207661, 207662, 207663, 207664, 207665, 207666, 207667, 207668, 207669, 207670) c6( ) c7(T, aC, fR, Or, fd, n, V, ad, ft, aE, aT, jj, ae, dx, afu, aW, Y, afF, GA, aq, kp, t, ik, bz, eM, cj, apv, m, lB, KS, B); #c1(PMCH) c2(NP_002665) c3(pro-MCH preproprotein) c4(130830) c5(130831, 130832, 130833, 130834, 130835, 130836) c6( ) c7(bM, EQ, fN, To, aj, QZ, bS, ct, v, Tg, yw, oW, K, qD, aDp, bGL, hl, eH, Hu, Z, eF, cm, bJ, BU, byx, qe, bal, Cz, pw, ER); #c1(PMEL) c2(NP_001186982) c3(melanocyte protein PMEL isoform 2 precursor) c4(78953) c5(78954, 78955, 78956, 78957, 78958, 78959, 78960, 78961) c6(78962, 78963) c7(T, if, DV, jh, fd, bB, qj, ci, ze, qN, aYs, ad, x, fX, xy, akU, kH, hD, h, bOZ, Xh, ae, cp, d, kj, je, Y, ff, en, g, kk, aoG, Wv, ck, iM, dc, lF, cj, ahC, k, lC, pC); #c1(PMEL) c2(XP_006719632) c3(melanocyte protein PMEL isoform X2) c4(292727, 292728) c5(292729, 292730, 292731, 292732, 292733, 292734, 292735, 292736, 292737, 292738, 292739, 292740, 292741, 292742, 292743, 292744, 292745, 292746, 292747, 292748) c6(292749, 292750, 292751, 292752, 292753, 292754) c7(T, if, DV, jh, fd, bB, qj, ci, ze, qN, aYs, ad, x, fX, xy, akU, kH, hD, h, bOZ, Xh, ae, cp, d, kj, je, Y, ff, en, g, kk, aoG, Wv, ck, iM, dc, lF, cj, ahC, k, lC, pC); #c1(PMEL) c2(XP_011536987) c3(melanocyte protein PMEL isoform X1) c4(334675, 334676) c5(334677, 334678, 334679, 334680, 334681, 334682, 334683, 334684, 334685, 334686, 334687, 334688, 334689, 334690, 334691, 334692) c6(334693, 334694, 334695, 334696) c7(T, if, DV, jh, fd, bB, qj, ci, ze, qN, aYs, ad, x, fX, xy, akU, kH, hD, h, bOZ, Xh, ae, cp, d, kj, je, Y, ff, en, g, kk, aoG, Wv, ck, iM, dc, lF, cj, ahC, k, lC, pC); #c1(PMEL) c2(XP_011536988) c3(melanocyte protein PMEL isoform X3) c4(334697) c5(334698, 334699, 334700, 334701, 334702, 334703, 334704, 334705) c6(334706, 334707) c7(T, if, DV, jh, fd, bB, qj, ci, ze, qN, aYs, ad, x, fX, xy, akU, KH, hD, h, bOZ, Xh, ae, cp, d, kj, je, Y, ff, en, g, kk, aoG, Wv, ck, iM, dc, lF, cj, ahC, k, lC, pC); #c1(PMEL) c2(XP_011536989) c3(melanocyte protein PMEL isoform X4) c4( ) c5(334708, 334709, 334710, 334711, 334712, 334713, 334714, 334715, 334716) c6(334717, 334718) c7(T, if, DV, jh, fd, bB, qj, ci, ze, qN, aYs, ad, x, fX, xy, akU, kH, hD, h, bOZ, Xh, ae, cp, d, kj, je, Y, ff, en, g, kk, aoG, Wv, ck, iM, dc, lF, cj, ahC, k, lC, pC); #c1(PMEPA1) c2(NP_001242905) c3(protein TMEPA1 isoform d) c4(89203) c5(89204, 89205, 89206, 89207, 89208, 89209, 89210) c6( ) c7(bM, T, NT, jh, bB, fr, ci, ad, av, iu, aT, K, an, ae, s, aW, PX, g, t, bz, ck, ac, bE, m, X); #c1(PMEPA1) c2(NP_064567) c3(protein TMEPA1 isoform a) c4(203004, 203005, 203006) c5(203007, 203008, 203009, 203010, 203011, 203012, 203013, 203014, 203015, 203016, 203017, 203018) c6( ) c7(bM, T, NT, jh, bB, fr, ci, ad, av, iu, aT, K, an, ae, s, aW, PX, g, t, bz, ck, ac, bE, m, X); #c1(PMEPA1) c2(NP_954638) c3(protein TMEPA1 isoform b) c4(261128) c5(261129, 261130, 261131, 261132, 261133, 261134, 261135, 261136) c6( ) c7(bM, T, NT, jh, bB, fr, ci, ad, av, iu, aT, K, an, ae, s, aW, PX, g, t, bz, ck, ac, bE, m, X); #c1(PMEPA1) c2(NP_954639) c3(protein TMEPA1 isoform c precursor) c4( ) c5(261137, 261138, 261139, 261140, 261141, 261142, 261143, 261144, 261145, 261146, 261147) c6( ) c7(bM, T, NT, jh, bB, fr, ci, ad, av, iu, aT, K, an, ae, s, aW, PX, g, t, bz, ck, ac, bE, m, X); #c1(PMF1) c2(NP_001186582) c3(polyamine-modulated factor 1 isoform 3) c4(77890) c5(77891, 77892, 77893, 77894, 77895, 77896) c6( ) c7(n, dL, eL); #c1(PMF1) c2(NP_001186583) c3(polyamine-modulated factor 1 isoform 1) c4(77897) c5(77898, 77899, 77900, 77901, 77902, 77903) c6( ) c7(n, dL, eL); #c1(PMF1) c2(NP_009152) c3(polyamine-modulated factor 1 isoform 2) c4(171154) c5(171155, 171156, 171157, 171158, 171159, 171160, (71161) c6( ) c7(n, dL, eL); #c1(PMF1-BGLAP) c2(NP_001186590) c3(PMF1-BGLAP protein isoform 1) c4(77904) c5(77905, 77906, 77907, 77908, 77909, 77910) c6( ) c7(n, dL, eL); #c1(PMF1-BGLAP) c2(NP_001186592) c3(PMF1-BGLAP protein isoform 3) c4(77911) c5(77912, 77913, 77914, 77915, 77916, 77917) c6( ) c7(n, dL, eL); #c1(PML) c2(NP_002666) c3(protein PML isoform 6) c4(130837) c5(130838, 130839, 130840, 130841, 130842, 130843, 130844) c6( ) c7(T, fR, if, bB, ci, mF, x, aXK, iv, K, bc, aW, alb, g, eF, cm, eM, iT, cj, k, m, bi, cst, NK, wu, n, qN, eW, no, h, eV, vx, on, pi, aq, cM, avf, Zj, hk, B, jp, DD, eX, fZ, aA, Kh, acn, AO, DK, aT, dz, j, ae, af, Uf, iU, vT, ev, aPf, lo, en, hf, t, f, ck, ac, au, bt, oA, css, jh, hF, ad, hX, fX, py, bdM, iY, wm, d, BN, ff, eH, rU, aF, hp, iR, bz, baa, bE, dX, bC, hE, kS, y); #c1(PML) c2(NP_150241) c3(protein PML isoform 1) c4(227925, 227926, 227927, 227928) c5(227929, 227930, 227931, 227932, 227933, 227934, 227935, 227936, 227937, 227938, 227939, 227940, 227941, 227942, 227943, 227944, 227945, 227946) c6( ) c7(T, fR, if, bB, ci, mF, x, aXK, iv, K, bc, aW, alb, g, eF, cm, eM, iT, cj, k, m, bi, cst, NK, wu, n, qN, eW, no, h, eV, vx, on, pi, aq, cM, avf, Zj, hk, B, jp, DD, eX, fZ, aA, Kh, acn, AO, DK, aT, dz, jj, ae, af, Uf, iU, vT, ev, aPf, lo, en, hf, t, f, ck, ac, au, bt, oA, css, jh, hF, ad, hX, fX, py, bdM, iY, wm, d, BN, ff, eH, rU, aF, hp, iR, bz, baa, bE, dx, bC, hE, kS, y); #c1(PML) c2(NP_150242) c3(protein PML isoform 9) c4(227947) c5(227948, 227949, 227950, 227951, 227952, 227953, 227954, 227955, 227956, 227957, 227958, 227959, 227960, 227961, 227962) c6( ) c7(T, FR, if, bB, ci, mF, x, aXK, iv, K, bc, aW, alb, g, eF, cm, eM, iT, cj, k, m, bi, cst, NK, wu, n, qN, eW, no, h, eV, vx, on, pi, aq, cM, avf, Zj, hk, B, jp, DD, eX, fZ, aA, Kh, acn, AO, DK, aT, dz, jj, ae, af, Uf, iU, vT, ev, aPf, lo, en, hf, t, f, ck, ac, au, bt, oA, css, jh, hF, ad, hX, fX, py, bdM, iY, wm, d, BN, ff, eH, rU, aF, hp, iR, bz, baa, bE, dX, bC, hE, kS, y); #c1(PML) c2(NP_150243) c3(protein PML isoform 2) c4(227963) c5(227964, 227965, 227966, 227967, 227968, 227969) c6( ) c7(T, FR, if, bB, ci, mF, x, aXK, iv, K, bc, aW, alb, g, eF, cm, eM, iT, cj, k, m, bi, cst, NK, wu, n, qN, eW, no, h, eV, vx, on, pi, aq, cM, avf, Zj, hk, B, jp, DD, eX, fZ, aA, Kh, acn, AO, DK, aT, dz, jj, ae, af, Uf, iU, vT, ev, aPf, lo, en, hf, t, f, ck, ac, au, bt, oA, css, jh, hF, ad, hX, fX, py, bdM, iY, wm, d, BN, ff, eH, rU, aF, hp, iR, bz, baa, bE, dX, bC, hE, kS, y); #c1(PML) c2(NP_150247) c3(protein PML isoform 5) c4(227970) c5(227971, 227972, 227973, 227974, 227975, 227976) c6( ) c7(T, FR, if, bB, ci, mF, x, aXK, iv, K, bc, aW, alb, g, eF, cm, eM, iT, cj, k, m, bi, cst, NK, wu, n, qN, eW, no, h, eV, vx, on, pi, aq, cM, avf, Zj, hk, B, jp, DD, eX, fZ, aA, Kh, acn, AO, DK, aT, dz, j, ae, af, Uf, iU, vT, ev, aPf, lo, en, hf, t, f, ck, ac, au, bt, oA, css, jh, hF, ad, hX, fX, py, bdM, iY, wm, d, BN, ff, eH, rU, aF, hp, iR, bz, baa, bE, dX, bC, hE, kS, y); #c1(PML) c2(NP_150249) c3(protein PML isoform 7) c4( ) c5(227977, 227978, 227979, 227980, 227981, 227982) c6( ) c7(T, FR, if, bB, ci, mF, x, aXK, iv, K, bc, aW, alb, g, eF, cm, eM, iT, cj, k, m, bi, cst, NK, wu, n, qN, eW, no, h, eV, vx, on, pi, aq, cM, avf, Zj, hk, B, jp, DD, eX, fZ, aA, Kh, acn, AO, DK, aT, dz, jj, ae, af, Uf, iU, vT, ev, aPf, lo, en, hf, t, f, ck, ac, au, bt, oA, css, jh, hF, ad, hX, fX, py, bdM, iY, wm, d, BN, ff, eH, rU, aF, hp, iR, bz, baa, bE, dX, bC, hE, kS, y); #c1(PML) c2(NP_150250) c3(protein PML isoform 8) c4(227983) c5(227984, 227985, 227986, 227987, 227988, 227989) c6( ) c7(T, fR, if, bB, ci, mF, x, aXK, iv, K, bc, aW, alb, g, eF, cm, eM, iT, cj, k, m, bi, cst, NK, wu, n, qN, eW, no, h, eV, vx, on, pi, aq, cM, avf, Zj, hk, B, jp, DD, eX, fZ, aA, Kh, acn, AO, DK, aT, dz, jj, ae, af, Uf, iU, vT, ev, aPf, lo, en, hf, t, f, ck, ac, au, bt, oA, css, jh, hF, ad, hX, fX, py, bdM, iY, wm, d, BN, ff, eH, rU, aF, hp, iR, bz, baa, bE, dX, bC, hE, kS, y); #c1(PML) c2(NP_150252) c3(protein PML isoform 10) c4(227990) c5(227991, 227992, 227993, 227994, 227995, 227996) c6( ) c7(T, FR, if, bB, ci, mF, x, aXK, iv, K, bc, aW, alb, g, eF, cm, eM, iT, cj, k, m, bi, cst, NK, wu, n, qN, eW, no, h, eV, vx, on, pi, aq, cM, avf, Zj, hk, B, jp, DD, eX, fZ, aA, Kh, acn, AO, DK, aT, dz, jj, ae, af, Uf, iU, vT, ev, aPf, lo, en, hf, t, f, ck, ac, au, bt, oA, css, jh, hF, ad, hX, fX, py, bdM, iY, wm, d, BN, ff, eH, rU, aF, hp, iR, bz, baa, bE, dX, bC, hE, kS, y); #c1(PML) c2(NP_150253) c3(protein PML isoform 11) c4(227997) c5(227998, 227999, 228000, 228001, 228002, 228003) c6( ) c7(T, fR, if, bB, ci, mF, x, aXK, iv, K, bc, aW, alb, g, eF, cm, eM, iT, cj, k, m, bi, cst, NK, wu, n, qN, eW, no, h, eV, vx, on, pi, aq, cM, avf, Zj, hk, B, jp, DD, eX, fZ, aA, Kh, acn, AO, DK, aT, dz, jj, ae, af, Uf, iU, vT, ev, aPf, lo, en, hf, t, f, ck, ac, au, bt, oA, css, jh, hF, ad, hX, fX, py, bdM, iY, wm, d, BN, ff, eH, rU, aF, hp, iR, bz, baa, bE, dX, bC, hE, kS, y); #c1(PMM1) c2(NP_002667) c3 (phosphomannomutase 1) c4(130845) c5(130846, 130847, 130848, 130849, 130850, 130851) c6( ) c7(MY, Lo, vi); #c1(PMM2) c2(NP_000294) c3(phosphomannomutase 2) c4(6651, 6652) c5(6653, 6654, 6655, 6656, 6657, 6658, 6659) c6( ) c7(bhy, bL, cfE, dH, il, SQ, dv, SM, akL, dq, CZ, Lo, Km, nA, xH, nC, MY, jr, de); #c1(PMM2) c2(XP_005255431) c3(phosphomannomutase 2 isoform X4) c4(273645) c5(273646, 273647, 273648, 273649, 273650, 273651, 273652, 273653, 273654, 273655, 273656) c6( ) c7(bhy, bL, cfE, dH, il, SQ, dv, SM, akL, dq, CZ, Lo, Km, nA, xH, nC, MY, jr, de); #c1(PMP22) c2(NP_000295) c3(peripheral myelin protein 22) c4(6660, 6661) c5(6662, 6663, 6664, 6665, 6666, 6667, 6668, 6669, 6670, 6671, 6672, 6673, 6674, 6675, 6676, 6677, 6678) c6( ) c7(cw, ach, T, dA, ob, aik, Pt, xF, bkY, csv, amp, bbn, My, yk, csx, n, aj, bze, ci, fu, bX, mF, arX, q, aR, eA, aLl, LH, ke, csA, no, csC, vJ, aT, UD, cfP, bl, ji, bUo, bU, csy, sX, dF, mJ, yr, apl, csB, mD, csz, bT, aW, xs, bhi, aFT, r, oV, Ls, aDm, eH, agU, g, hf, eF, cbm, iV, pb, dr, dR, bkZ, sk, EW, csu, Hp, ql, bkW, bgy, csw, ig, mG, AH, bee, UF, bCG, afO); #c1(PMP22) c2(XP_011522245) c3(peripheral myelin protein 22 isoform X1) c4(316882) c5(316883, 316884, 316885, 316886, 316887) c6( ) c7(cw, ach, T, dA, ob, aik, Pt, xF, bkY, csv, amp, bbn, My, yk, csx, n, aj, bze, ci, fu, bX, mF, arX, q, aR, eA, aLl, LH, ke, csA, no, csC, vJ, aT, UD, cfP, bl, ji, buo, bU, csy, sX, dF, mJ, yr, apl, csB, mD, csz, bT, aW, xs, bhi, aFT, r, oV, Ls, aDm, eH, agU, g, hf, eF, cbm, iV, pb, dr, dR, bkZ, sk, EW, csu, Hp, ql, bkW, bgy, csw, ig, mG, AH, bee, UF, bCG, afO); #c1(PMPCA) c2(NP_001269873) c3(mitochondrial-processing peptidase subunit alpha isoform 2) c4(102996) c5(102997, 102998, 102999, 103000, 103001, 103002) c6( ) c7(bSf, Tp); #c1(PMPCA) c2(NP_055975) c3(mitochondrial-processing peptidase subunit alpha isoform 1 precursor) c4(184826, 184827) c5(184828, 184829, 184830, 184831, 184832, 184833, 184834, 184835, 184836, 184837, 184838, 184839) c6( ) c7(bSf, Tp); #c1(PMPCA) c2(XP_005266116) c3(mitochondrial-processing peptidase subunit alpha isoform X1) c4(279908) c5( ) c6( ) c7(bSf, Tp); #c1(PMPCA) c2(XP_011516719) c3(mitochondrial-processing peptidase subunit alpha isoform X2) c4( ) c5(310173, 310174, 310175, 310176, 310177, 310178) c6( ) c7(bSf, Tp); #c1(PMPCB) c2(NP_004270) c3(mitochondrial-processing peptidase subunit beta precursor) c4(144743) c5(144744, 144745, 144746, 144747, 144748, 144749) c6( ) c7(iU, T, ff, en); #c1(PMPCB) c2(XP_005250774) c3(mitochondrial-processing peptidase subunit beta isoform X1) c4(270534) c5(270535, 270536, 270537, 270538, 270539, 270540) c6( ) c7(iU, T, ff, en); #c1(PMS1) c2(NP_000525) c3(PMS1 protein homolog 1 isoform a) c4(11856, 11857, 11858) c5(11859, 11860, 11861, 11862, 11863, 11864, 11865, 11866, 11867, 11868, 11869, 11870, 11871, 11872) c6( ) c7(T, aC, fM, jh, aA, cz, fr, eA, q, ad, aE, aT, kH, dL, ae, je, BO, Y, r, kp, aF, g, t, bz, Bv, iM, at, dW, m, fh); #c1(PMS1) c2(NP_001121615) c3(PMSl protein homolog 1 isoform b) c4(45863) c5(45864, 45865, 45866, 45867, 45868, 45869) c6( ) c7(T, aC, fM, jh, aA, cz, fr, eA, q, ad, aE, aT, kH, dl, ae, je, BO, Y, r, kp, aF, g, t, bz, Bv, iM, at, dW, m, fh); #c1(PMS1) c2(NP_001121616) c3(PMS1 protein homolog 1 isoform c) c4(45870, 45871) c5(45872, 45873, 45874, 45875, 45876, 45877) c6( ) c7(T, aC, fM, jh, aA, cz, fr, eA, q, ad, aE, aT, kH, dL, ae, je, BO, Y, r, kp, aF, g, t, bz, Bv, iM, at, dW, m, fh); #c1(PMS1) c2(NP_001276338) c3 (PMS1 protein homolog 1 isoform d) c4( ) c5(108129, 108130, 108131, 108132, 108133, 108134, 108135, 108136, 108137, 108138, 108139, 108140, 108141) c6( ) c7(T, aC, fM, jh, aA, cz, fr, eA, q, ad, aE, aT, KH, dL, ae, je, BO, Y, r, kp, aF, g, t, bz, Bv, iM, at, dW, m, fh); #c1(PMS1) c2(XP_005246706) c3(PMSl protein homolog 1 isoform X3) c4(267373) c5( ) c6( ) c7(T, aC, fM, jh, aA, cz, fr, eA, q, ad, aE, aT, kH, dL, ae, je, BO, Y, r, kp, aF, g, t, bz, Bv, iM, at, dW, m, fh); #c1(PMS1) c2(XP_011509657) c3(PMSl protein homolog 1 isoform X4) c4(300231) c5( ) c6( ) c7(T, aC, fM, jh, aA, cz, fr, eA, q, ad, aE, aT, kH, dL, ae, je, BO, Y, r, kp, aF, g, t, bz, Bv, iM, at, dW, m, fh); #c1(PMS1) c2(XP_011509659) c3(PMSl protein homolog 1 isoform X6) c4( ) c5(300232, 300233, 300234, 300235, 300236, 300237, 300238, 300239, 300240, 300241, 300242, 300243, 300244) c6( ) c7(T, aC, fM, jh, aA, cz, fr, eA, q, ad, aE, aT, kH, dL, ae, je, BO, Y, r, kp, aF, g, t, bz, Bv, iM, at, dW, m, fh); #c1(PMS2) c2(NP_000526) c3(mismatch repair endonuclease PMS2 isoform a) c4(11873, 11874, 11875) c5(11876, 11877, 11878, 11879, 11880, 11881, 11882, 11883, 11884, 11885, 11886, 11887, 11888, 11889, 11890, 11891, 11892, 11893) c6( ) c7(T, gU, Fj, jh, bB, n, fr, ci, qN, q, ad, er, bv, b, eO, aT, vH, YB, dL, KV, ae, jf, oS, d, BO, s, ND, aW, Y, NE, YC, r, aJK, Kf, cl, t, bz, bF, sS, alC, csD, dB, bE, gX, hL, at, oh, m, i, X); #c1(PMS2) c2(XP_006715805) c3(mismatch repair endonuclease PMS2 isoform X2) c4(289792) c5(289793, 289794, 289795, 289796, 289797, 289798) c6( ) c7(T, gU, Fj, jh, bB, n, fr, ci, qN, q, ad, er, bv, b, eO, aT, vH, YB, dL, KV, ae, jf, oS, d, BO, s, ND, aW, Y, NE, YC, r, aJK, Kf, cl, t, bz, bF, sS, alC, csD, dB, bE, gX, hL, at, oh, m, i, X); #c1(PMS2) c2(XP_011513732) c3(mismatch repair endonuclease PMSZ isoform X4) c4(306011) c5(306012, 306013, 306014, 306015, 306016, 306017) c6( ) c7(T, gU, Fj, jh, bB, n, fr, ci, qN, q, ad, er, bv, b, eO, aT, vH, YB, dL, KV, ae, jf, oS, d, BO, s, ND, aW, Y, NE, YC, r, aJK, Kf, cl, t, bz, bF, sS, alC, csD, dB, bE, gX, hl, at, oh, m, i, X); #c1(PMVK) c2(NP_006547) c3(phosphomevalonate kinase) c4(166156) c5(166157, 166158, 166159, 166160, 166161, 166162) c6( ) c7(bzi); #c1(PNCK) c2(NP_001034671) c3(calcium/calmodulin-dependent protein kinase type 18 isoform a) c4(32038) c5(32039, 32040, 32041, 32042, 32043, 32044, 32045, 32046, 32047) c6( ) c7(py); #c1(PNCK) c2(NP_001129212) c3(calcium/calmodulin-dependent protein kinase type 1B isoform b) c4(51207) c5(51208, 51209, 51210, 51211, 51212, 51213, 51214, 51215, 51216) c6( ) c7(py); #c1(PNCK) c2(XP_011529409) c3(calcium/calmodulin-dependent protein kinase type 1B isoform X1) c4(324787, 324788, 324789, 324790, 324791, 324792) c5(324793, 324794, 324795) c6( ) c7(py); #c1(PNCK) c2(XP_011529410) c3(calcium/calmodulin-dependent protein kinase type 1B isoform X2) c4( ) c5(324796, 324797, 324798) c6( ) c7(py); #c1(PNCK) c2(XP_011529415) c3(calcium/calmodulin-dependent protein kinase type 18 isoform X3) c4(324799) c5(324800, 324801, 324802, 324803, 324804, 324805, 324806, 324807, 324808, 324809, 324810, 324811, 324812, 324813, 324814, 324815, 324816, 324817, 324818, 324819, 324820, 324821, 324822, 324823, 324824, 324825, 324826, 324827, 324828, 324829, 324830, 324831, 324832, 324833, 324834, 324835, 324836, 324837, 324838, 324839, 324840, 324841, 324842, 324843, 324844, 324845, 324846, 324847, 324848, 324849, 324850, 324851) c6( ) c7(py); #c1(PNKD) c2(NP_001070867) c3(probable hydrolase PNKD isoform 3 precursor) c4(34865) c5(34866, 34867, 34868, 34869, 34870, 34871) c6( ) c7(T, aZ, cV, cz, fr, aR, ahR, Hx, hd, dV, aT, aU, bsE, K, bc, aW, kp, t, xw, c, m, L); #c1(PNKD) c2(NP_056303) c3 (probable hydrolase PNKD isoform 1 precursor) c4(187014) c5(187015, 187016, 187017, 187018, 187019, 187020) c6( ) c7(T, aZ, cV, cz, fr, aR, ahR, Hx, hd, dV, aT, aU, bsE, K, bc, aW, kp, t, xw, c, m, L); #c1(PNKD) c2(NP_072094) c3 (probable hydrolase PNKD isoform 2) c4(212695) c5(212696, 212697, 212698, 212699, 212700, 212701) c6( ) c7(T, aZ, cV, cz, fr, aR, ahR, Hx, hd, dV, aT, aU, bsE, K, bc, aW, kp, t, xw, c, m, L); #c1(PNKP) c2(NP_009185) c3(bifunctional polynucleotide phosphatase/kinase) c4(171366) c5(171367, 171368, 171369, 171370, 171371, 171372) c6( ) c7(aR, s, ob, X, csE, U, n, csF, K, QK, V, ne, aij); #c1(PNLDC1) c2(NP_001258791) c3(poly(A)-specific ribonuclease PARN-like domain-containing protein 1 isoform 1) c4( ) c5(96545) c6( ) c7(dy, ac); #c1(PNLDC1) c2(NP_775787) c3(poly(A)-specific ribonuclease PARN-like domain-containing protein 1 isoform 2 precursor) c4(250311) c5(250312, 250313, 250314, 250315, 250316) c6( ) c7(dy, ac); #c1(PNL1P) c2(NP_000927) c3(pancreatic triacylglycerol lipase precursor) c4(17402) c5(17403, 17404, 17405, 17406, 17407, 17408) c6( ) c7(bL, ar, asp, csG, v, ci, cm); #c1(PNLlPRP2) c2(NP_005387) c3 (pancreatic lipase-related protein 2 precursor) c4(155630) c5(155631, 155632, 155633, 155634, 155635, 155636) c6( ) c7(ar, aE); #c1(PNMA1) c2(NP_006020) c3 (paraneoplastic antigen Ma1) c4(161218) c5(161219, 161220, 161221, 161222, 161223, 161224) c6( ) c7(ke, csH); #c1(PNMA2) c2(NP_009188) c3(paraneoplastic antigen Ma2) c4(171409) c5(171410, 171411, 171412, 171413, 171414, 171415) c6( ) c7(T, nG, fd); #c1(PNMT) c2(NP_002677) c3(phenylethanolamine N-methyltransferase) c4(130852, 130853) c5(130854, 130855, 130856, 130857, 130858, 130859, 130860) c6( ) c7(bxy, ac, aW, aw, bo, VB, tV, aT, tc, cz, v, K, bq, cm, V, af, qa); #c1(PNMT) c2(XP_011523211) c3(phenylethanolamine N-methyltransferase isoform X1) c4( ) c5(317726, 317727, 317728, 317729, 317730) c6( ) c7(bxy, ac, aW, aw, bo, VB, tV, aT, tc, cz, v, K, bq, cm, V, af, qa); #c1(PNN) c2(NP_002678) c3(pinin) c4(130861) c5(130862, 130863, 130864, 130865, 130866, 130867, 130868) c6( ) c7(x, T, bS, k, ct, Z, bt, hq); #c1(PNO1) c2(NP_064528) c3(RNA-binding protein PNO1) c4(202871) c5(202872, 202873, 202874, 202875, 202876, 202877) c6( ) c7(bM, T, aC, ha, jh, aA, bB, tc, TM, Vd, ci, nA, lR, fr, hF, eA, aR, ad, hX, eW, ft, aE, WY, ds, SV, aT, kf, Ak, dV, h, K, an, brV, fb, ae, af, dx, iL, d, vT, aW, Y, ff, eZ, nf, aq, aaE, aF, Ku, en, iR, bz, pb, QA, bN, dR, ck, rR, kP, c, eM, bE, Qu, dh, mx, U, aEs, rx, xh, arg, B); #c1(PNOC) c2(NP_006219) c3(prepronociceptin isoform 1 preproprotein) c4(163039, 163040) c5(163041, 163042, 163043, 163044, 163045, 163046, 163047, 163048, 163049, 163050, 163051, 163052) c6( ) c7(eY, T, VR, ot, csl, Lg, bQC, gu, UN, C, cm, lB, xt, FT); #c1(PNP) c2(NP_000261) c3(purine nucleoside phosphorylase) c4(5881, 5882) c5(5883, 5884, 5885, 5886, 5887, 5888, 5889, 5890, 5891, 5892, 5893, 5894) c6( ) c7(bxC, T, dD, dA, ea, jh, eq, eA, ad, eJ, eW, x, aE, dV, aT, h, dL, ae, af, eL, cp, cY, bA, aW, Y, oV, dq, CQ, beP, t, cm, dr, Cv, bE, U, aiD, cS, k, Pk, B); #c1(PNPLA1) c2(NP_001139188) c3(patatin-like phospholipase domain-containing protein 1 isoform 2) c4(58890) c5(58891, 58892, 58893, 58894, 58895) c6( ) c7(fn, v, csJ, lQ); #c1(PNPLA1) c2(NP_001139189) c3(patatin-like phospholipase domain-containing protein 1 isoform 3) c4(58896, 58897, 58898) c5(58899, 58900, 58901, 58902, 58903, 58904, 58905, 58906, 58907, 58908, 58909, 58910, 58911, 58912) c6( ) c7(fn, v, csJ, lQ); #c1(PNPLA1) c2(NP_775947) c3(patatin-like phospholipase domain-containing protein 1 isoform 1) c4( ) c5(250773, 250774, 250775, 250776, 250777) c6( ) c7(fn, v, csJ, lQ); #c1(PNPLA2) c2(NP_065109) c3(patatin-like phospholipase domain-containing protein 2) c4(203966) c5(203967, 203968, 203969, 203970, 203971, 203972) c6( ) c7(bL, cZ, T, dD, ea, e, ol, aj, v, eq, bSh, cd, J, ad, eA, aE, oj, aT, aU, cy, jy, K, zp, dd, ae, Um, DA, kl, lR, bNp, aW, Y, eH, fc, Mz, aq, eF, t, og, dr, iO, Fi, Be, vy, gZ, JL, arf, nw, m, cP, du, ei, Zw, vt, auN, B); #c1(PNPLA3) c2(NP_079501) c3(patatin-like phospholipase domain-containing protein 3) c4(218410) c5(218411, 218412, 218413, 218414, 218415, 218416, 218417) c6( ) c7(bL, T, dD, ea, al, qQ, jx, aj, dG, v, jE, eq, J, kL, GK, hX, l, F, u, h, jy, qM, dF, avl, A, fc, hl, dq, lo, aq, iR, gk, cP, ei, B); #c1(PNPLA4) c2(NP_001166143) c3(patatin-like phospholipase domain-containing protein 4 isoform 2) c4( ) c5(68319, 68320, 68321, 68322, 68323) c6( ) c7(v); #c1(PNPLA4) c2(NP_004641) c3(patatin-like phospholipase domain-containing protein 4 isoform 1 precursor) c4(148439, 148440, 148441) c5(148442, 148443, 148444, 148445, 148446, 148447, 148448, 148449, 148450, 148451, 148452, 148453, 148454, 148455, 148456, 148457, 148458) c6( ) c7(v); #c1(PNPLA5) c2(NP_001171146) c3(patatin-like phospholipase domain-containing protein 5 isoform 2) c4(69931, 69932) c5(69933, 69934, 69935, 69936, 69937, 69938, 69939, 69940, 69941, 69942, 69943, 69944, 69945, 69946, 69947, 69948) c6( ) c7(v, aw); #c1(PNPLA5) c2(NP_620169) c3(patatin-like phospholipase domain-containing protein 5 isoform 1) c4(237452) c5(237453, 237454, 237455, 237456, 237457, 237458) c6( ) c7(v, aw); #c1(PNPLA6) c2(NP_001159583) c3(neuropathy target esterase isoform a) c4(64360) c5(64361, 64362, 64363, 64364, 64365, 64366) c6( ) c7(bEn, c, aV, ob, uT, dM, Ol, csL, U, cm, lo, bl, fu, bX, aEZ, csK, aR); #c1(PNPLA6) c2(NP_001159584) c3(neuropathy target esterase isoform c) c4(64367) c5(64368, 64369, 64370, 64371, 64372, 64373) c6( ) c7(bEn, c, aV, ob, uT, dM, OI, csL, U, cm, lo, bl, fu, bX, aEZ, csK, aR); #c1(PNPLA6) c2(NP_001159585) c3(neuropathy target esterase isoform b) c4(64374) c5(64375, 64376, 64377, 64378, 64379, 64380, 64381, 64382, 64383, 64384, 64385, 64386, 64387, 64388, 64389, 64390) c6( ) c7(bEn, c, aV, ob, uT, dM, Ol, csL, U, cm, lo, bl, fu, bX, aEZ, csK, aR); #c1(PNPLA6) c2(NP_001159586) c3(neuropathy target esterase isoform d) c4(64391) c5( ) c6( ) c7(bEn, c, aV, ob, uT, dM, Ol, csL, U, cm, lo, bl, fu, bX, aEZ, csK, aR); #c1(PNPLA8) c2(NP_001242937) c3(calcium-independent phospholipase A2-gamma isoform 1) c4(89283) c5(89284, 89285, 89286, 89287, 89288, 89289, 89290, 89291, 89292, 89293, 89294, 89295, 89296) c6( ) c7(q, g, r); #c1(PNPLA8) c2(NP_001242938) c3(calcium-independent phospholipase A2-gamma isoform 2) c4(89297, 89298, 89299, 89300) c5(89301, 89302, 89303, 89304, 89305, 89306, 89307, 89308, 89309, 89310, 89311, 89312, 89313, 89314, 89315, 89316, 89317, 89318, 89319, 89320, 89321, 89322, 89323) c6( ) c7(q, g, r); #c1(PNPLA8) c2(NP_001242939) c3(calcium-independent phospholipase A2-gamma isoform 3) c4(89324) c5(89325, 89326, 89327, 89328, 89329) c6( ) c7(q, g, r); #c1(PNPLA8) c2(XP_011514576) c3(calcium-independent phospholipase A2-gamma isoform X2) c4( ) c5(307283, 307284) c6( ) c7(q, g, r); #c1(PNPLA8) c2(XP_011514577) c3(calcium-independent phospholipase A2-gamma isoform X3) c4( ) c5(307285) c6( ) c7(q, g, r); #c1(PNP0) c2(NP_060599) c3(pyridoxine-5′-phosphate oxidase) c4(196768, 196769, 196770, 196771) c5(196772, 196773, 196774, 196775, 196776, 196777, 196778, 196779, 196780, 196781, 196782, 196783, 196784, 196785, 196786, 196787, 196788, 196789) c6( ) c7(csM, T, Cq, ha, cz, aq, lm, cB, B, cC); #c1(PNP0) c2(XP_005257557) c3(pyridoxine-5′-phosphate oxidase isoform X1) c4( ) c5(275267) c6( ) c7(csM, T, Cq, ha, cz, aq, lm, cB, B, cC); #c1(PNP0) c2(XP_011523270) c3(pyridoxine-5′-phosphate oxidase isoform X2) c4( ) c5(317785, 317786, 317787, 317788, 317789) c6( ) c7(csM, T, Cq, ha, cz, aq, lm, cB, B, cC); #c1(PNPT1) c2(NP_149100) c3(polyribonucleotide nucleotidyltransferase 1, mitochondrial precursor) c4(227143) c5(227144, 227145, 227146, 227147, 227148, 227149, 227150, 227151, 227152, 227153) c6( ) c7(T, aE, c, oz, csO, oe, Y, ev, csN, at, cd, bZ, Fb, cm, V, Bh, B); #c1(PNRC1) c2(NP_006804) c3(proline-rich nuclear receptor coactivator 1) c4(168245, 168246) c5(168247, 168248, 168249, 168250, 168251, 168252, 168253, 168254, 168255, 168256, 168257, 168258, 168259, 168260, 168261, 168262) c6( ) c7(B, vi); #c1(POC1A) c2(NP_001155052) c3(POC1 centriolar protein homolog A isoform 2) c4(61386) c5(61387, 61388, 61389, 61390, 61391, 61392) c6( ) c7(aW, csQ, al, csP, csR, csS, Df); #c1(POC1A) c2(NP_001155053) c3(POC1 centriolar protein homolog A isoform 3) c4( ) c5(61393, 61394, 61395, 61396, 61397) c6( ) c7(aW, csQ, aT, csP, csR, csS, Df); #c1(POC1A) c2(NP_056241) c3(POC1 centriolar protein homolog A isoform 1) c4(186674, 186675) c5(186676, 186677, 186678, 186679, 186680, 186681, 186682) c6( ) c7(aW, csQ, aT, csP, csR, csS, Df); #c1(POC1B) c2(NP_001186706) c3(POC1 centriolar protein homolog B isoform b) c4(78378) c5(78379, 78380, 78381, 78382, 78383, 78384, 78385, 78386, 78387, 78388, 78389, 78390, 78391, 78392, 78393, 78394, 78395) c6( ) c7(nC, aC); #c1(POC1B) c2(NP_758440) c3(POC1 centriolar protein homolog B isoform a) c4(249150, 249151, 249152) c5(249153, 249154, 249155, 249156, 249157, 249158, 249159) c6( ) c7(nC, aC); #c1(POC5) c2(NP_001092741) c3(centrosomal protein POC5 isoform 1) c4(39598) c5(39599, 39600, 39601, 39602, 39603, 39604) c6( ) c7(fq); #c1(POC5) c2(NP_689621) c3(centrosomal protein POC5 isoform 2) c4(243317) c5(243318, 243319, 243320, 243321, 243322, 243323) c6( ) c7(fq); #c1(POC5) c2(XP_005248493) c3(centrosomal protein POC5 isoform X1) c4(268411) c5( ) c6( ) c7(fq); #c1(POC5) c2(XP_005248494) c3(centrosomal protein POC5 isoform X4) c4( ) c5(268412, 268413, 268414, 268415, 268416, 268417, 268418, 268419, 268420, 268421, 268422, 268423, 268424, 268425, 268426, 268427, 268428, 268429) c6( ) c7(fq); #c1(POC5) c2(XP_011541460) c3(centrosomal protein POC5 isoform X2) c4(340038) c5(340039, 340040, 340041, 340042, 340043, 340044, 340045, 340046, 340047, 340048) c6( ) c7(fq); #c1(POC5) c2(XP_011541462) c3(centrosomal protein POC5 isoform X3) c4(340049, 340050) c5( ) c6( ) c7(fq); #c1(PODXL) c2(NP_001018121) c3(podocalyxin isoform 1 precursor) c4(27378, 27379) c5(27380, 27381, 27382, 27383, 27384, 27385, 27386, 27387, 27388, 27389, 27390, 27391) c6( ) c7(T, n, fr, uE, aY, q, ad, jz, dY, aT, ae, d, B, aW, ff, r, en, bu, g, t, bE, eD, X); #c1(PODXL) c2(NP_005388) c3(podocalyxin isoform 2 precursor) c4(155637, 155638, 155639) c5(155640, 155641, 155642, 155643, 155644, 155645, 155646, 155647, 155648, 155649, 155650, 155651, 155652, 155653, 155654, 155655, 155656, 155657) c6( ) c7(T, n, fr, uE, aY, q, ad, jz, dY, aT, ae, d, B, aW, ff, r, en, bu, g, t, bE, eD, X); #c1 (POF1B) c2(NP_079197) c3(protein POF1B) c4(217437, 217438) c5(217439, 217440, 217441, 217442, 217443, 217444, 217445, 217446, 217447, 217448, 217449, 217450) c6( ) c7(c, il, pQ, aeS, csT, lQ, zZ); #c1(POFUT1) c2(NP_056167) c3(GDP-fucose protein O-fucosyltransferase 1 isoform 1 precursor) c4(186067, 186068) c5(186069, 186070, 186071, 186072, 186073, 186074, 186075, 186076, 186077, 186078, 186079, 186080) c6( ) c7(rF, eA, csU); #c1(POFUT1) c2(NP_758436) c3(GDP-fucose protein O-fucosyltransferase 1 isoform 2 precursor) c4(249143) c5(249144, 249145, 249146, 249147, 249148, 249149) c6( ) c7(rF, eA, csU); #c1(POFUT2) c2(NP_056042) c3(GDP-fucose protein O-fucosyltransferase 2 isoform A precursor) c4(185273) c5(185274, 185275, 185276, 185277, 185278, 185279, 185280) c6( ) c7(bL, y, A, j, cz); #c1(POFUT2) c2(NP_598368) c3(GDP-fucose protein O-fucosyltransferase 2 isoform C precursor) c4(235450) c5(235451, 235452, 235453, 235454, 235455, 235456) c6( ) c7(bL, y, A, j, cz); #c1(POGK) c2(NP_060012) c3(pogo transposable element with KRAB domain) c4(193645, 193646, 193647, 193648) c5(193649, 193650, 193651, 193652, 193653, 193654, 193655, 193656, 193657, 193658) c6( ) c7(bA, cz); #c1(POGK) c2(XP_005245428) c3(pogo transposable element with KRAB domain isoform X3) c4( ) c5(266570, 266571, 266572, 266573, 266574, 266575) c6( ) c7(bA, cz); #c1(POGK) c2(XP_011508117) c3(pogo transposable element with KRAB domain isoform X2) c4( ) c5(298596, 298597, 298598, 298599, 298600, 298601) c6( ) c7(bA, cz); #c1(POGLUT1) c2(NP_689518) c3(protein 0-glucosyltransferase 1 precursor) c4(242963) c5(242964, 242965, 242966, 242967, 242968, 242969) c6( ) c7(csV, bw, bt, hp, br); #c1(POLA1) c2(NP_058633) c3(DNA polymerase alpha catalytic subunit) c4(192857) c5(192858, 192859, 192860, 192861, 192862, 192863) c6( ) c7(eF, Wq, eH, nA); #c1(POLA1) c2(XP_005274609) c3(DNA polymerase alpha catalytic subunit isoform X1) c4(285186) c5(285187, 285188, 285189, 285190, 285191, 285192) c6( ) c7(eF, Wq, eH, nA); #c1(POLA2) c2(NP_002680) c3(DNA polymerase alpha subunit B) c4(130896) c5(130897, 130898, 130899, 130900, 130901, 130902) c6( ) c7(dL); #c1(POLA2) c2(XP_011543180) c3(DNA polymerase alpha subunit B isoform X2) c4(342012) c5(342013, 342014, 342015, 342016, 342017, 342018) c6( ) c7(dL); #c1(POLB) c2(NP_002681) c3(DNA polymerase beta) c4(130903, 130904, 130905) c5(130906, 130907, 130908, 130909, 130910, 130911) c6( ) c7(GI, T, aC, wu, ht, aA, bB, hy, ci, bX, V, q, ad, eA, ip, Ok, aT, h, lG, Gw, dL, K, wm, ae, af, eL, wr, aW, eZ, r, af, t, f, bz, dr, bF, es, dB, bE, cj, at, m, Gu, it, X); #c1(POLB) c2(XP_005273592) c3(DNA polymerase beta isoform X1) c4(284681) c5(284682, 284683, 284684, 284685, 284686, 284687) c6( ) c7(GI, T, aC, wu, ht, aA, bB, hy, ci, bX, V, q, ad, eA, ip, Qk, aT, h, lG, Gw, dL, K, wm, ae, af, eL, wr, aW, eZ, r, aF, t, f, bz, dr, bF, es, dB, bE, cj, at, m, Gu, it, X); #c1(POLB) c2(XP_005273594) c3(DNA polymerase beta isoform X3) c4(284688) c5(284689, 284690, 284691, 284692, 284693, 284694) c6( ) c7(GI, T, aC, wu, ht, aA, bB, hy, ci, bX, V, q, ad, eA, ip, Qk, aT, h, IG, Gw, dL, K, wm, ae, af, eL, wr, aW, eZ, r, aF, t, f, bz, dr, bF, es, dB, bE, cj, at, m, Gu, it, X); #c1(POLB) c2(XP_005273595) c3(DNA polymerase beta isoform X4) c4( ) c5(284695, 284696, 284697, 284698, 284699, 284700, 284701) c6( ) c7(GI, T, aC, wu, ht, aA, bB, hy, ci, bX, V, q, ad, eA, ip, Qk, aT, h, lG, Gw, dL, K, wm, ae, af, eL, wr, aW, eZ, r, aF, t, f, bz, dr, bF, es, dB, bE, cj, at, m, Gu, it, X); #c1(POLB) c2(XP_005273597) c3(DNA polymerase beta isoform X5) c4( ) c5(284702, 284703, 284704, 284705, 284706) c6( ) c7(GI, T, aC, wu, ht, aA, bB, hy, ci, bX, V, q, ad, eA, ip, Ok, aT, h, lG, Gw, dL, K, wm, ae, af, eL, wr, aW, eZ, r, aF, t, f, bz, dr, bF, es, dB, bE, cj, at, m, Gu, it, X); #c1(POLDI) c2(NP_001243778) c3(DNA polymerase delta catalytic subunit) c4(91703, 91704, 91705) c5(91706, 91707, 91708, 91709, 91710, 91711, 91712, 91713, 91714, 91715, 91716, 91717) c6( ) c7(csX, T, os, zt, atS, jh, fd, bB, auq, oZ, j, ci, V, fK, q, ad, UE, dP, eA, aT, h, dL, K, j, ae, af, eL, jf, d, B, aW, Y, r, aq, csW, cl, t, ik, bz, GR, ek, ck, bF, ac, eM, bP, bE, do, hL, hR, at, aSp, m, bC, lM, bt, KS, X, L); #c1(POLD2) c2(NP_001243808) c3(DNA polymerase delta subunit 2 isoform 1) c4( ) c5(91787, 91788, 91789, 91790, 91791, 91792, 91793, 91794, 91795, 91796, 91797, 91798, 91799, 91800, 91801) c6( ) c7(ci, dL, m); #c1(POLD2) c2(XP_011513733) c3(DNA polymerase delta subunit 2 isoform X1) c4(306018, 306019, 306020, 306021) c5(306022, 306023, 306024, 306025, 306026, 306027, 306028, 306029, 306030, 306031, 306032, 306033, 306034) c6( ) c7(ci, dL, m); #c1(POLD3) c2(NP_006582) c3(DNA polymerase delta subunit 3) c4(166386, 166387, 166388, 166389) c5(166390, 166391, 166392, 166393, 166394, 166395) c6( ) c7(T, bE, aW, ds, Pg, aLJ, aT, r, bB, aq, dL, iw, K, q, eA, h, ik, ae, B, ad); #c1(POLD3) c2(XP_005273773) c3(DNA polymerase delta subunit 3 isoform X2) c4( ) c5(284828, 284829, 284830, 284831, 284832, 284833) c6( ) c7(T, bE, aW, ds, Pg, aLJ, aT, r, bB, aq, dL, iw, K, q, eA, h, ik, ae, B, ad); #c1(POLD3) c2(XP_011543036) c3(DNA polymerase delta subunit 3 isoform X1) c4( ) c5(341771, 341772, 341773, 341774, 341775, 341776, 341777, 341778, 341779, 341780, 341781, 341782) c6( ) c7(T, bE, aW, ds, Pg, aLJ, aT, r, bB, aq, dL, iw, K, q, eA, h, iK, ae, B, ad); #c1(POLD4) c2(NP_001243799) c3(DNA polymerase delta subunit 4 isoform 2) c4(91751) c5(91752, 91753, 91754, 91755, 91756, 91757, 91758, 91759, 91760, 91761, 91762) c6( ) c7(ck, T, ft, iM, lR, je, eH, dV, aT, ew, eA, bX, eF, bt, bz, zo, ao); #c1(POLD4) c2(NP_066996) c3(DNA polymerase delta subunit 4 isoform 1) c4(208072, 208073) c5(208074, 208075, 208076, 208077, 208078, 208079, 208080) c6( ) c7(ck, T, ft, iM, IR, je, eH, dV, aT, ew, eA, bX, eF, bt, bz, zo, ao); #c1(POLDIP3) c2(NP_001265586) c3(polymerase delta-interacting protein 3 isoform 3) c4( ) c5(98910) c6( ) c7(c, vi); #c1(POLDIP3) c2(NP_115687) c3(polymerase delta-interacting protein 3 isoform 1) c4(223548, 223549, 223550) c5(223551, 223552, 223553, 223554, 223555, 223556) c6( ) c7(c, vi); #c1(POLDIP3) c2(NP_835237) c3(polymerase delta-interacting protein 3 isoform 2) c4(253421, 253422) c5(253423, 253424, 253425, 253426, 253427, 253428, 253429, 253430, 253431, 253432, 253433, 253434, 253435) c6( ) c7(c, vi); #c1(POLDIP3) c2(XP_011528759) c3(polymerase delta-interacting protein 3 isoform X1) c4(324060) c5(324061, 324062, 324063, 324064, 324065, 324066, 324067, 324068, 324069, 324070, 324071, 324072, 324073, 324074, 324075, 324076) c6( ) c7(c, vi); #c1(POLE) c2(NP_006222) c3(DNA polymerase epsilon catalytic subunit A) c4(163053, 163054, 163055, 163056, 163057) c5(163058, 163059, 163060, 163061, 163062, 163063, 163064, 163065, 163066, 163067, 163068, 163069, 163070, 163071, 163072, 163073, 163074, 163075, 163076, 163077, 163078, 163079, 163080, 163081, 163082, 163083, 163084, 163085, 163086, 163087) c6( ) c7(av, T, cF, csY, AM, hl, aT, cj, n, r, aA, dL, ci, q, cl, af, QK, V, it, csZ); #c1(POLE) c2(XP_011533103) c3(DNA polymerase epsilon catalytic subunit A isoform X7) c4( ) c5(329288, 329289, 329290, 329291, 329292, 329293) c6( ) c7(av, T, cF, csY, AM, hL, aT, cj, n, r, aA, dL, ci, q, cl, aF, QK, V, it, csZ); #c1(POLE) c2(XP_011533104) c3(DNA polymerase epsilon catalytic subunit A isoform X8) c4(329294) c5( ) c6( ) c7(av, T, cF, csY, AM, hl, aT, cj, n, r, aA, dL, ci, q, cl, af, QK, V, it, csZ); #c1(POLE2) c2(NP_001184259) c3(DNA polymerase epsilon subunit 2 isoform 2) c4(75599) c5(75600, 75601, 75602, 75603, 75604, 75605) c6( ) c7(dL); #c1(POLE2) c2(NP_001184260) c3(DNA polymerase epsilon subunit 2 isoform 3) c4(75606) c5(75607, 75608, 75609, 75610, 75611, 75612) c6( ) c7(dL); #c1(POLE2) c2(NP_002683) c3(DNA polymerase epsilon subunit 2 isoform 1) c4(130912) c5(130913, 130914, 130915, 130916, 130917, 130918) c6( ) c7(dL); #c1(POLE3) c2(NP_059139) c3(DNA polymerase epsilon subunit 3) c4(193244) c5(193245, 193246, 193247, 193248, 193249, 193250, 193251, 193252, 193253, 193254) c6( ) c7(Ke, NL, fG, aA, h, ey, aoG); #c1(POLE4) c2(NP_063949) c3(DNA polymerase epsilon subunit 4) c4(202401) c5(202402, 202403, 202404, 202405, 202406, 202407) c6( ) c7(ck, T, iM, lR, je, eH, dV, aT, eA, bX, eF, bt, bz, zo, ao); #c1(POLG) c2(NP_002684) c3(DNA polymerase subunit gamma-1) c4(130919) c5(130920, 130921, 130922, 130923, 130924, 130925, 130926, 130927, 130928, 130929) c6( ) c7(T, zW, aR, il, K, bU, dF, aW, ir, GR, wT, AV, mx, U, ctc, m, hh, gU, n, cc, h, dL, bV, awL, clK, A, KH, Fr, cA, SE, bk, gv, cB, Al, awH, cz, GC, aNG, v, j, bX, bgR, V, MR, hb, aT, bK, bq, an, ae, ajU, Ft, EG, kl, ND, aeS, aPf, bho, cta, cte, kC, bcT, bN, ac, ctb, awN, mG, ob, Pt, ha, ctg, ctf, fu, aoz, ad, cth, oj, lm, iL, yr, EH, KM, ctd, akQ, ag, awk, Lb); #c1(POLG2) c2(NP_009146) c3(DNA polymerase subunit gamma-2, mitochondrial) c4(171140) c5(171141, 171142, 171143, 171144, 171145, 171146) c6( ) c7(aR, y, F, xE, A, eL, oj, cti, bt, cds, dV, dL, h, zW, lM, awL, kC, bz, X, amW, uQ); #c1(POLH) c2(NP_001278899) c3(DNA polymerase eta isoform 3) c4(110843) c5(110844, 110845, 110846, 110847, 110848, 110849, 110850, 110851, 110852, 110853, 110854, 110855) c6( ) c7(eW, T, BN, Y, yk, CT, aT, hy, dL, adg, ctj, ne, dr); #c1(POLH) c2(NP_006493) c3(DNA polymerase eta isoform 1) c4(165700, 165701, 165702) c5(165703, 165704, 165705, 165706, 165707, 165708, 165709) c6( ) c7(eW, T, BN, Y, yk, CT, aT, hy, dL, adg, ctj, ne, dr); #c1(POLH) c2(XP_005249243) c3(DNA polymerase eta isoform X2) c4( ) c5(269148, 269149, 269150, 269151, 269152) c6( ) c7(eW, T, BN, Y, yk, CT, aT, hy, dL, adg, ctj, ne, dr); #c1(POLH) c2(XP_011513000) c3(DNA polymerase eta isoform X1) c4( ) c5(304450, 304451, 304452) c6( ) c7(eW, T, BN, Y, yk, CT, aT, hy, dL, adg, ctj, ne, dr); #c1(POL1) c2(NP_009126) c3(DNA polymerase iota) c4(170923, 170924) c5(170925, 170926, 170927, 170928, 170929, 170930, 170931, 170932, 170933, 170934, 170935, 170936, 170937, 170938, 170939, 170940) c6( ) c7(bM, T, sO, aW, if, ff, cn, cj, aT, it, uJ, dL, v, ci, en, hu, qN, pk, eL, Zj, ad); #c1(POLK) c2(NP_057302) c3(DNA polymerase kappa) c4(189985, 189986, 189987, 189988, 189989, 189990) c5(189991, 189992, 189993, 189994, 189995, 189996, 189997, 189998, 189999, 190000, 190001, 190002) c6( ) c7(aW, r, aT, dL, K, bz, q); #c1(POLK) c2(XP_006714715) c3(DNA polymerase kappa isoform X5) c4( ) c5(288039, 288040, 288041, 288042, 288043, 288044, 288045, 288046, 288047, 288048) c6( ) c7(aW, r, aT, dL, K, bz, q); #c1(POLK) c2(XP_011541770) c3(DNA polymerase kappa isoform X4) c4(340354) c5(340355, 340356, 340357, 340358, 340359, 340360, 340361, 340362, 340363, 340364, 340365, 340366, 340367, 340368, 340369, 340370, 340371, 340372, 340373, 340374, 340375, 340376, 340377, 340378) c6( ) c7(aW, r, aT, dL, K, bz, q); #c1(POLL) c2(NP_001167555) c3(DNA polymerase lambda isoform a) c4(69181, 69182, 69183, 69184, 69185, 69186, 69187) c5(69188, 69189, 69190, 69191, 69192, 69193, 69194, 69195, 69196, 69197, 69198, 69199, 69200, 69201, 69202, 69203, 69204, 69205, 69206, 69207, 69208, 69209, 69210, 69211, 69212, 69213, 69214, 69215, 69216, 69217, 69218, 69219, 69220, 69221, 69222, 69223, 69224, 69225, 69226, 69227, 69228, 69229) c6( ) c7(ae, T, av, gU, Pf, aW, aT, cj, r, dL, K, q, bz, B, ad, MG); #c1(POLL) c2(XP_006717839) c3(DNA polymerase lambda isoform X10) c4(291379) c5(291380, 291381, 291382, 291383, 291384, 291385) c6( ) c7(ae, T, av, gU, Pf, aW, aT, cj, r, dL, K, q, bz, B, ad, MG); #c1(POLL) c2(XP_011537956) c3(DNA polymerase lambda isoform X4) c4(335472) c5(335473, 335474, 335475, 335476, 335477, 335478) c6( ) c7(ae, T, av, gU, Pf, aW, aT, cj, r, dL, K, q, bz, B, ad, MG); #c1(POLL) c2(XP_011537958) c3(DNA polymerase lambda isoform X5) c4( ) c5(335479, 335480, 335481, 335482, 335483, 335484, 335485) c6( ) c7(ae, T, av, gU, Pf, aW, aT, cj, r, dL, K, q, bz, B, ad, MG); #c1(POLL) c2(XP_011537960) c3(DNA polymerase lambda isoform X6) c4(335486, 335487) c5(335488, 335489, 335490) c6( ) c7(ae, T, av, gU, Pf, aW, aT, cj, r, dL, K, q, bz, B, ad, MG); #c1(POLL) c2(XP_011537962) c3(DNA polymerase lambda isoform X7) c4( ) c5(335491, 335492, 335493, 335494, 335495, 335496, 335497, 335498, 335499, 335500, 335501, 335502, 335503) c6( ) c7(ae, T, av, gU, Pf, aW, aT, cj, r, dL, K, q, bz, B, ad, MG); #c1(POLL) c2(XP_011537969) c3 (DNA polymerase lambda isoform X13) c4( ) c5(335504, 335505, 335506, 335507, 335508, 335509) c6( ) c7(ae, T, av, gU, Pf, aW, aT, cj, r, dL, K, q, bz, B, ad, MG); #c1(POLM) c2(NP_001271259) c3(DNA-directed DNA/RNA polymerase mu isoform 2) c4(103758) c5(103759, 103760, 103761, 103762, 103763, 103764) c6( ) c7(av, auM, jh, it, dL, ci, pk, bz, eA); #c1(POLM) c2(NP_001271260) c3 (DNA-directed DNA/RNA polymerase mu isoform 3) c4(103765) c5(103766, 103767, 103768, 103769, 103770, 103771) c6( ) c7(av, auM, jh, it, dL, ci, pk, bz, eA); #c1(POLM) c2(NP_037416) c3(DNA-directed DNA/RNA polymerase mu isoform 1) c4(175402, 175403, 175404, 175405) c5(175406, 175407, 175408, 175409, 175410, 175411) c6( ) c7(av, auM, jh, it, dL, ci, pk, bz, eA); #c1(POLM) c2(XP_005249768) c3(DNA-directed DNA/RNA polymerase mu isoform X11) c4( ) c5(269816) c6( ) c7(av, auM, jh, it, dL, ci, pk, bz, eA); #c1(POLM) c2(XP_006715755) c3(DNA-directed DNA/RNA polymerase mu isoform X2) c4( ) c5(289778) c6( ) c7(av, auM, jh, it, dL, ci, pk, bz, eA); #c1(POLM) c2(XP_011513586) c3(DNA-directed DNA/RNA polymerase mu isoform X16) c4( ) c5(305899) c6( ) c7(av, auM, jh, it, dL, ci, pk, bz, eA); #c1(POLM) c2(XP_011513587) c3(DNA-directed DNA/RNA polymerase mu isoform X17) c4( ) c5(305900) c6( ) c7(av, auM, jh, it, dL, ci, pk, bz, eA); #c1(POLM) c2(XP_011513588) c3(DNA-directed DNA/RNA polymerase mu isoform X18) c4( ) c5(305901, 305902, 305903, 305904, 305905) c6( ) c7(av, auM, jh, it, dL, ci, pk, bz, eA); #c1(POLM) c2(XP_011513589) c3(DNA-directed DNA/RNA polymerase mu isoform X19) c4( ) c5(305906, 305907, 305908, 305909, 305910, 305911, 305912, 305913, 305914, 305915, 305916) c6( ) c7(av, auM, jh, it, dL, ci, pk, bz, eA); #c1(POLN) c2(NP_861524) c3(DNA polymerase nu) c4(255639, 255640, 255641) c5(255642, 255643, 255644, 255645, 255646, 255647, 255648, 255649, 255650, 255651, 255652, 255653, 255654, 255655, 255656, 255657, 255658, 255659, 255660, 255661, 255662, 255663) c6( ) c7(Y, af); #c1(POLQ) c2(NP_955452) c3(DNA polymerase theta) c4(261745, 261746, 261747) c5(261748, 261749, 261750, 261751, 261752, 261753, 261754, 261755, 261756, 261757, 261758, 261759, 261760, 261761, 261762, 261763, 261764, 261765) c6( ) c7(T, aW, yk, CT, aT, dL, adg, ctj, eW); #c1(POLQ) c2(XP_011510655) c3(DNA polymerase theta isoform X10) c4( ) c5(301093) c6( ) c7(T, aW, yk, CT, aT, dL, adg, ctj, eW); #c1(POLR1A) c2(NP_056240) c3(DNA-directed RNA polymerase 1 subunit RPA1) c4(186660, 186661) c5(186662, 186663, 186664, 186665, 186666, 186667, 186668, 186669, 186670, 186671, 186672, 186673) c6( ) c7(j, Kh); #c1(POLR1B) c2(NP_001131076) c3(DNA-directed RNA polymerase 1 subunit RPA2 isoform 2) c4(52579) c5(52580, 52581, 52582, 52583, 52584, 52585) c6( ) c7(q, K, r); #c1(POLR1B) c2(NP_001269701) c3(DNA-directed RNA polymerase 1 subunit RPA2 isoform 3) c4(102603, 102604) c5(102605, 102606, 102607, 102608, 102609, 102610, 102611, 102612, 102613) c6( ) c7(q, K, r); #c1(POLR1B) c2(NP_001269703) c3(DNA-directed RNA polymerase 1 subunit RPA2 isoform 4) c4(102625, 102626) c5(102627, 102628, 102629, 102630, 102631, 102632, 102633, 102634, 102635, 102636) c6( ) c7(q, K, r); #c1(POLR1B) c2(NP_001269705) c3(DNA-directed RNA polymerase 1 subunit RPA2 isoform 5) c4(102658) c5(102659, 102660, 102661, 102662, 102663, 102664) c6( ) c7(q, K, r); #c1(POLR1B) c2(NP_061887) c3(DNA-directed RNA polymerase 1 subunit RPA2 isoform 1) c4(200990) c5(200991, 200992, 200993, 200994, 200995, 200996, 200997, 200998, 200999, 201000, 201001) c6( ) c7(q, K, r); #c1(POLR1C) c2(NP_976035) c3(DNA-directed RNA polymerases I and III subunit RPAC1) c4(262837, 262838) c5(262839, 262840, 262841, 262842, 262843, 262844, 262845, 262846, 262847, 262848, 262849, 262850) c6( ) c7(ctk, Xg); #c1(POLR1C) c2(XP_005249548) c3(DNA-directed RNA polymerases I and III subunit RPAC1 isoform X1) c4(269563) c5(269564, 269565, 269566, 269567, 269568, 269569) c6( ) c7(ctk, Xg); #c1(POLR1D) c2(NP_057056) c3(DNA-directed RNA polymerases I and III subunit RPAC2 isoform 1) c4(188662) c5(188663, 188664, 188665, 188666, 188667, 188668, 188669, 188670, 188671, 188672) c6( ) c7(dy, lB, ctl, ac, Xg); #c1(POLR1D) c2(NP_689918) c3(DNA-directed RNA polymerases I and III subunit RPAC2 isoform 2) c4(244364) c5(244365, 244366, 244367, 244368, 244369, 244370) c6( ) c7(dy, lB, ctl, ac, Xg); #c1(POLR2A) c2(NP_000928) c3(DNA-directed RNA polymerase II subunit RPB1) c4(17409) c5(17410, 17411, 17412, 17413, 17414, 17415, 17416) c6( ) c7(T, py, aA, dL, fr, aF); #c1(POLR2B) c2(NP_000929) c3(DNA-directed RNA polymerase II subunit RPB2 isoform 1) c4(17417) c5( ) c6( ) c7(le, dL, W); #c1(POLR2B) c2(NP_001290198) c3(DNA-directed RNA polymerase II subunit RPB2 isoform 3) c4( ) c5(115114, 115115, 115116, 115117, 115118, 115119, 115120, 115121, 115122, 115123) c6( ) c7(le, dL, W); #c1(POLR2C) c2(NP_116558) c3(DNA-directed RNA polymerase II subunit RPB3) c4(226172) c5(226173, 226174, 226175, 226176, 226177, 226178) c6( ) c7(dl, Jr); #c1(POLR2D) c2(NP_004796) c3(DNA-directed RNA polymerase 1 subunit RPB4) c4(149928) c5(149929, 149930, 149931, 149932, 149933, 149934) c6( ) c7(bL, ctm, aPt, A, F, nx, al, mg, Mz, h, aj, mf, dL, ahl, v, ei, ae, fK, J, ad); #c1(POLR2E) c2(NP_002686) c3(DNA-directed RNA polymerases I, lI, and III subunit RPABC1) c4(130930) c5(130931, 130932, 130933, 130934, 130935, 130936) c6( ) c7(cm, dL); #c1(POLR2F) c2(NP_001288058) c3(DNA-directed RNA polymerases I, lI, and III subunit RPABC2 isoform 2) c4( ) c5(113458, 113459, 113460, 113461, 113462, 113463) c6( ) c7(dl, ci); #c1(POLR2F) CZ(NP_001288059) c3(DNA-directed RNA polymerases I, lI, and III subunit RPABC2 isoform 3) c4(113464) c5(113465, 113466, 113467, 113468, 113469, 113470) c6( ) c7(dl, ci); #c1(POLR2F) c2(NP_001288060) c3(DNA-directed RNA polymerases I, lI, and III subunit RPABC2 isoform 4) c4( ) c5(113471, 113472, 113473, 113474, 113475, 113476) c6( ) c7(dl, ci); #c1(POLR2F) c2(NP_068809) c3(DNA-directed RNA polymerases I, lI, and III subunit RPABC2 isoform 1) c4(210688) c5( ) c6( ) c7(dl, ci); #c1(POLR2F) c2(XP_011528537) c3(DNA-directed RNA polymerases I, lI, and III subunit RPABC2 isoform X1) c4(323672) c5( ) c6( ) c7(dl, ci); #c1(POLR2G) c2(NP_002687) c3(DNA-directed RNA polymerase II subunit RPB7) c4(130937) c5(130938, 130939, 130940, 130941, 130942, 130943) c6( ) c7(es, cr, pl, ks); #c1(POLR2H) c2(NP_001265643) c3(DNA-directed RNA polymerases I, lI, and III subunit RPABC3 isoform 4) c4( ) c5(99083, 99084, 99085, 99086) c6( ) c7(dL); #c1(POLR2H) c2(NP_006223) c3(DNA-directed RNA polymerases I, lI, and III subunit RPABC3 isoform 2) c4(163088, 163089) c5(163090, 163091, 163092, 163093, 163094, 163095, 163096, 163097, 163098, 163099, 163100, 163101, 163102, 163103, 163104, 163105) c6( ) c7(dL); #c1(POLR2J) c2(NP_006225) c3(DNA-directed RNA polymerase II subunit RPB11-a) c4(163106) 5(163107, 163108, 163109, 163110, 163111, 163112) c6( ) c7(dL); #c1(POLR2J) c2(XP_005250509) c3(DNA-directed RNA polymerase Il subunit RPB11-a isoform X1) c4(270360) c5(270361, 270362, 270363, 270364, 270365, 270366) c6( ) c7(dL); #c1(POLR2K) c2(NP_005025) c3(DNA-directed RNA polymerases I, lI, and III subunit RPABC4) c4(152265) c5(152266, 152267, 152268, 152269, 152270, 152271) c6( ) c7(t, dL); #c1(POLR2M) c2(NP_001018112) c3(protein GRINL1A isoform 2) c4(27371) c5(27372, 27373, 27374, 27375, 27376, 27377) c6( ) c7(ae, K, ad); #c1(POLR2M) c2(NP_056347) c3(protein GRINL1A isoform 1) c4(187200, 187201) c5(187202, 187203, 187204, 187205, 187206, 187207, 187208, 187209, 187210, 187211, 187212) c6( ) c7(ae, K, ad); #c1(POLR3A) c2(NP_008986) c3(DNA-directed RNA polymerase III subunit RPC1) c4(170071, 170072, 170073) c5(170074, 170075, 170076, 170077, 170078, 170079, 170080, 170081, 170082, 170083, 170084, 170085, 170086, 170087, 170088, 170089, 170090, 170091) c6( ) c7(T, ctn, uo, lR, UF, cb, pb); #c1(POLR3B) c2(NP_001154180) c3(DNA-directed RNA polymerase III subunit RPC2 isoform 2) c4( ) c5(61040, 61041, 61042, 61043, 61044) c6( ) c7(cb, cto, aml, nA, UF, agy, EZ); #c1(POLR3B) c2(NP_060552) c3(DNA-directed RNA polymerase III subunit RPC2 isoform 1) c4(196530, 196531) c5(196532, 196533, 196534, 196535, 196536, 196537, 196538) c6( ) c7(cb, cto, aml, nA, UF, agy, EZ); #c1(POLR3E) c2(NP_001244962) c3(DNA-directed RNA polymerase III subunit RPC5 isoform 2) c4(92461) c5(92462, 92463, 92464, 92465, 92466) c6( ) c7(T, bkf, Y, h, CQ, fh); #c1(POLR3E) c2(NP_001244963) c3(DNA-directed RNA polymerase III subunit RPC5 isoform 4) c4(92467) c5(92468, 92469, 92470, 92471, 92472) c6( ) c7(T, bkf, Y, h, CQ, fh); #c1(POLR3E) c2(NP_001244965) c3(DNA-directed RNA polymerase III subunit RPC5 isoform 5) c4(92473) c5(92474, 92475, 92476, 92477, 92478) c6( ) c7(T, bkf, Y, h, CQ, fh); #c1(POLR3E) c2(NP_060589) c3(DNA-directed RNA polymerase III subunit RPC5 isoform 1) c4(196699) c5(196700, 196701, 196702, 196703, 196704) c6( ) c7(T, bkf, Y, h, CQ, fh); #c1(POLR3K) c2(NP_057394) c3(DNA-directed RNA polymerase III subunit RPC10) c4(190546) c5(190547, 190548, 190549, 190550, 190551, 190552) c6( ) c7(aA); #c1(POLRMT) c2(NP_005026) c3(DNA-directed RNA polymerase, mitochondrial precursor) c4(152272, 152273) c5(152274, 152275, 152276, 152277, 152278, 152279) c6( ) c7(awL, dL); #c1(POLRMT) c2(XP_005259637) c3(DNA-directed RNA polymerase, mitochondrial isoform X1) c4( ) c5(276219, 276220) c6( ) c7(awL, dL); #c1(POLRMT) c2(XP_011526373) c3(DNA-directed RNA polymerase, mitochondrial isoform X2) c4( ) c5(320662, 320663) c6( ) c7(awL, dL); #c1(POM121) c2(NP_001244119) c3(nuclear envelope pore membrane protein POM 121 isoform 1) c4(91930) c5(91931, 91932, 91933, 91934, 91935, 91936, 91937, 91938, 91939, 91940, 91941) c6( ) c7(rc, iU); #c1(POM121) c2(NP_742017) c3(nuclear envelope pore membrane protein POM 121 isoform 2) c4(247877) c5(247878, 247879, 247880, 247881, 247882, 247883, 247884, 247885) c6( ) c7(rc, iU); #c1(POM121) c2(XP_005250783) c3(nuclear envelope pore membrane protein POM 121 isoform X2) c4(270541) c5(270542, 270543, 270544, 270545, 270546, 270547, 270548, 270549, 270550, 270551, 270552, 270553, 270554, 270555, 270556, 270557, 270558) c6( ) c7(rc, iU); #c1(POM121) c2(XP_011515032) c3(nuclear envelope pore membrane protein POM 121 isoform X4) c4( ) c5(307808) c6( ) c7(rc, iU); #c1(POMC) c2(XP_011531219) c3(pro-opiomelanocortin isoform X1) c4(327194, 327195, 327196, 327197, 327198, 327199) c5(327200, 327201, 327202, 327203, 327204, 327205, 327206, 327207, 327208, 327209, 327210, 327211, 327212, 327213, 327214, 327215, 327216, 327217, 327218, 327219, 327220, 327221, 327222, 327223, 327224, 327225, 327226, 327227, 327228) c6(327229) c7(T, uk, Fj, FR, azf, acz, vf, yY, bO, Ja, qj, ci, rm, afl, bh, GK, ctq, tg, aex, aE, il, axy, aX, pX, Gb, Fo, iX, vg, iv, K, qM, wt, aVL, dF, cp, li, yo, avH, bn, Fe, fc, bbe, hJ, DL, zV, yX, bbk, Gs, Ji, cj, uo, QF, aVN, OD, lS, xh, hh, nD, Uc, sQ, lT, n, aj, afE, ct, ctr, ml, nE, fr, qW, cd, bbc, ai, biN, rc, ke, co, ft, aMV, cc, ec, aoh, cty, Nn, adX, h, jy, gl, uV, he, cC, ak, avP, ctw, kj, A, Cw, fJ, Rj, iw, AQ, fQ, ctt, Hu, baZ, ctv, ctz, cM, fq, bJ, hq, bLF, ok, adZ, hk, bo, RJ, hR, HB, ut, lB, jd, adG, bbl, bL, eR, DD, aG, yk, jx, qQ, rf, cz, ti, v, j, C, aql, wy, V, eA, J, ga, cts, TI, nv, aVM, afQ, ctx, aT, ctp, cy, bK, UN, bbd, an, hK, Fg, bZN, bA, hM, kl, s, baY, aMG, nO, Y, apk, cu, en, t, FT, bbK, bN, cT, sS, Ta, Dy, c, alC, hc, dy, EY, fD, UF, lw, bbJ, aaT, ra, pY, aZ, iW, ha, jN, jh, OE, OR, aET, bS, dG, MA, ceY, OH, gb, ad, hX, qC, av, hd, ib, avL, ctu, baX, vc, bj, lQ, bHm, Yi, mu, hZ, gj, yr, mD, vj, ff, bBO, cr, gu, EZ, Z, bz, qa, dR, OG, zr, dJ, Rm, ckY, ei, oQ, y, MN, aUg, X, hv, pC); #c1(POMGNT1) c2(NP_001277059) c3(protein O-linked-mannose beta-1,2-N-acetylglucosaminyltransferase 1 isoform 4) c4( ) c5(108996) c6( ) c7(ctB, ul, ctC, ctA, xs, km, bcZ, nA, Wq, lf); #c1(POMGNT1) c2(XP_011540062) c3(protein O-linked-mannose beta-1,2-N-acetylglucosaminyltransferase 1 isoform X2) c4(338361) c5(338362, 338363, 338364, 338365, 338366, 338367) c6( ) c7(ctB, ul, ctC, ctA, xs, km, bcZ, nA, Wq, lf); #c1(POMGNT2) c2(NP_116195) c3(protein I-linked-mannose beta-1,4-N-acetylglucosaminyltransferase 2 precursor) c4(225680) c5(225681, 225682, 225683, 225684, 225685, 225686, 225687, 225688, 225689, 225690, 225691, 225692, 225693, 225694) c6(225695, 225696, 225697, 225698) c7(ctE, he, xs, ctD); #c1(POMK) c2(NP_115613) c3(protein O-mannose kinase) c4(223368) c5(223369, 223370, 223371, 223372, 223373, 223374) c6( ) c7(ctF); #c1(POMP) c2(NP_057016) c3(proteasome maturation protein) c4(188533) c5(188534, 188535, 188536, 188537, 188538, 188539) c6( ) c7(bL, ctG, aW, om, aT, lQ, K, Zm, aHG); #c1(POMT1) c2(NP_001070834) c3(protein O-mannosyl-transferase 1 isoform c) c4( ) c5(34853) c6( ) c7(VC, cc, QK, hb, amJ, dB, xs, km, Ak, ctH, nA, ctI, eA, lf); #c1(POMT1) c2(NP_001129585) c3(protein O-mannosyl-transferase 1 isoform b) c4(51902, 51903, 51904, 51905) c5(51906, 51907, 51908, 51909, 51910, 51911, 51912, 51913, 51914, 51915, 51916, 51917, 51918, 51919, 51920, 51921, 51922, 51923, 51924, 51925, 51926, 51927, 51928) c6( ) c7(VC, cc, QK, hb, amJ, dB, xs, km, Ak, ctH, nA, ctl, eA, lf); #c1(POMT1) c2(NP_001129586) c3(protein O-mannosyl-transferase 1 isoform d) c4(51929, 51930, 51931) c5(51932, 51933, 51934, 51935, 51936, 51937, 51938, 51939, 51940, 51941, 51942, 51943, 51944, 51945, 51946, 51947) c6( ) c7(VC, cc, QK, hb, amJ, dB, xs, km, Ak, ctH, nA, ctl, eA, lf); #c1(POMT1) c2(XP_005272213) c3(protein O-mannosyl-transferase 1 isoform X1) c4(283764, 283765, 283766) c5(283767, 283768, 283769, 283770, 283771, 283772, 283773) c6( ) c7(VC, cc, QK, hb, amJ, dB, xs, km, Ak, ctH, nA, ctl, eA, lf); #c1(POMT1) c2(XP_005272219) c3(protein O-mannosyl-transferase 1 isoform X12) c4( ) c5(283774) c6( ) c7(VC, cc, QK, hb, amJ, dB, xs, km, Ak, ctH, nA, ctl, eA, lf); #c1(POMT1) c2(XP_006716995) c3 (protein O-mannosyl-transferase 1 isoform X7) c4(290609) c5( ) c6( ) c7(VC, cc, QK, hb, amJ, dB, xs, km, Ak, ctH, nA, ctl, eA, lf); #c1(POMT1) c2(XP_011516443) c3(protein O-mannosyl-transferase 1 isoform X3) c4( ) c5(309692, 309693, 309694, 309695, 309696, 309697) c6( ) c7(VC, cc, QK, hb, amJ, dB, xs, km, Ak, ctH, nA, ctl, eA, lf); #c1(POMT1) c2(XP_011516445) c3(protein O-mannosyl-transferase 1 isoform X6) c4(309698) c5(309699) c6( ) c7(VC, cc, QK, hb, amJ, dB, xs, km, Ak, ctH, nA, ctl, eA, lf); #c1(POMT1) c2(XP_011516446) c3 (protein O-mannosyl-transferase 1 isoform X8) c4( ) c5(309700, 309701, 309702, 309703, 309704, 309705, 309706, 309707, 309708, 309709, 309710, 309711, 309712, 309713, 309714, 309715, 309716, 309717) c6( ) c7(VC, cc, QK, hb, amJ, dB, xs, km, Ak, ctH, nA, ctl, eA, lf); #c1(POMT1) c2(XP_011516448) c3(protein O-mannosyl-transferase 1 isoform X10) c4( ) c5(309718, 309719, 309720, 309721, 309722, 309723, 309724, 309725) c6( ) c7(VC, cc, QK, hb, amJ, dB, xs, km, Ak, ctH, nA, ctl, eA, lf); #c1(POMT1) c2(XP_011516449) c3(protein O-mannosyl-transferase 1 isoform X1l) c4(309726) c5(309727, 309728, 309729, 309730, 309731, 309732) c6( ) c7(VC, cc, QK, hb, amJ, dB, xs, km, Ak, ctH, nA, ctl, eA, lf); #c1(POMT2) c2(NP_037514) c3 (protein O-mannosyl-transferase 2) c4(175966) c5(175967, 175968, 175969, 175970, 175971, 175972) c6( ) c7(VC, ctL, ctJ, ctk, hb, FB, xs, km, nA, QK, Wq, lf); #c1(POMZP3) c2(NP_036362) c3(POM121 and ZP3 fusion protein isoform 1) c4(173238, 173239) c5(173240, 173241, 173242, 173243, 173244, 173245) c6( ) c7(rc); #c1(POMZP3) c2(NP_694537) c3(POM121 and ZP3 fusion protein isoform 2) c4( ) c5(245087, 245088, 245089, 245090, 245091, 245092, 245093, 245094, 245095, 245096) c6( ) c7(rc); #c1(PON1) c2(NP_000437) c3(serum paraoxonase/arylesterase 1 precursor) c4(8974, 8975) c5(8976, 8977, 8978, 8979, 8980, 8981, 8982, 8983, 8984, 8985, 8986, 8987) c6( ) c7(bM, T, fR, ea, xo, e, al, bO, fd, gf, Vd, ip, Na, Gt, dY, Fo, Gb, M, K, oi, qD, dQ, eN, avn, Us, hm, DA, bbC, aW, hl, as, ahv, uC, y, ctO, lp, wE, Ji, U, vy, uo, m, pw, bbv, dD, gU, sQ, tp, Ol, aw, cQ, aj, ct, bmA, aLc, TB, ft, no, ec, Bj, gq, h, eV, dL, ey, uV, ZN, wn, Gc, ctN, hi, A, xq, fe, aq, wZ, cM, hq, ar, kT, W, cA, dB, Fi, bm, at, uz, ut, ctP, lB, B, bL, M, dA, aCw, mr, eR, oe, jx, qQ, eX, cz, sM, cR, hy, v, j, wo, V, J, rS, MP, aT, yu, cy, Nc, bK, an, rz, avl, ne, ae, af, eu, jf, kl, aCs, r, lo, en, wA, t, bN, ck, sk, rR, ac, c, pZ, bbA, hc, DW, hr, fP, hO, mf, cS, dy, ctM, bt, ao, vi, sY, aZ, iW, jh, vr, mw, bS, uu, fu, dG, eq, iG, hF, aue, ad, dZ, dP, fU, nH, av, F, oW, LA, vw, xp, ji, lQ, tt, vl, sj, uQ, uX, BN, lR, Ub, cH, cW, ff, gC, cr, eZ, hp, aot, Z, iV, Hv, ek, iq, gk, nb, aS, ei, Pk, st, rk); #c1(PON2) c2(NP_000296) c3(serum paraoxonase/arylesterase 2 isoform 1) c4(6679, 6680, 6681) c5(6682, 6683, 6684, 6685, 6686, 6687, 6688, 6689, 6690, 6691, 6692, 6693, 6694, 6695, 6696, 6697, 6698, 6699) c6( ) c7(kV, bL, bM, dD, aCw, eR, fR, sQ, ea, df, e, aw, cz, cQ, aj, j, Vd, bX, v, eq, J, dZ, dY, ec, M, cy, K, lQ, af, eu, hm, uX, A, lR, cH, hl, fe, hp, wA, cM, ek, kT, ac, c, wE, vy, bm, uo, gZ, ut, dy, Pk, gq, ao); #c1(PON2) c2(NP_001018171) c3(serum paraoxonase/arylesterase 2 isoform 2) c4(27515) c5(27516, 27517, 27518, 27519, 27520, 27521) c6( ) c7(kV, bL, bM, dD, aCw, eR, fR, sQ, ea, df, e, aw, cz, cd, aj, j, Vd, bX, v, eq, J, dZ, dY, ec, M, cy, K, lQ, af, eu, hm, uX, A, lR, CH, hl, fe, hp, wA, cM, ek, kT, ac, c, wE, vy, bm, uo, gZ, ut, dy, Pk, gq, ao); #c1(PON3) c2(NP_000931) c3(serum paraoxonase/lactonase 3) c4(17418) c5(17419, 17420, 17421, 17422, 17423, 17424) c6( ) c7(T, dD, ac, c, dY, ea, aw, hl, eZ, h, fe, v, j, K, Vd, hp, eq, af); #c1(POP1) c2(NP_001139332) c3(ribonucleases P/MRP protein subunit POP1) c4(59205) c5(59206, 59207, 59208, 59209, 59210, 59211, 59212, 59213, 59214, 59215) c6( ) c7(awB, dt, uo, adz); #c1(POP1) c2(XP_011515102) c3(ribonucleases P/MRP protein subunit POP1 isoform X1) c4(307966) c5( ) c6( ) c7(awB, dt, uo, adz); #c1(POP1) c2(XP_011515103) c3(ribonucleases P/MRP protein subunit POP1 isoform X2) c4( ) c5(307967, 307968, 307969, 307970, 307971, 307972) c6( ) c7(awB, dt, uo, adz); #c1(POP4) c2(NP_006618) c3(ribonuclease P protein subunit p29) c4(166652, 166653) c5(166654, 166655, 166656, 166657, 166658, 166659, 166660, 166661, 166662, 166663, 166664, 166665, 166666 166667, 166668, 166669) c6( ) c7(fe, t); #c1(POPDC3) c2(NP_071756) c3(popeye domain-containing protein 3) c4(211957, 211958) c5(211959, 211960, 211961, 211962, 211963, 211964, 211965, 211966, 211967, 211968, 211969, 211970) c6( ) c7(V, aF, adz, aA); #c1(POR) c2(NP_000932) c3(NADPH-cytochrome P450 reductase) c4(17425, 17426, 17427, 17428, 17429, 17430, 17431, 17432, 17433, 17434, 17435) c5(17436, 17437, 17438, 17439, 17440, 17441, 17442, 17443, 17444, 17445, 17446, 17447, 17448, 17449, 17450, 17451, 17452, 17453, 17454, 17455, 17456, 17457, 17458, 17459, 17460, 17461, 17462, 17463, 17464, 17465, 17466, 17467, 17468, 17469, 17470, 17471, 17472, 17473, 17474, 17475, 17476, 17477, 17478, 17479, 17480, 17481, 17482, 17483, 17484, 17485, 17486, 17487, 17488, 17489, 17490, 17491, 17492, 17493, 17494, 17495, 17496, 17497, 17498, 17499, 17500, 17501, 17502, 17503, 17504, 17505) c6( ) c7(qC, bN, T, av, zw, s, azf, aW, ctQ, aT, aeS, h, aj, dL, zV, K, avF, bz, J, xt, On); #c1(PORCN) c2(NP_001269096) c3(protein-cysteine N-palmitoyltransferase porcupine isoform F) c4( ) c5(100438, 100439, 100440, 100441, 100442) c6( ) c7(qC, av, T, afH, aW, aT, h, avF, aj, zV, arx, bHi, bz, J); #c1(PORCN) c2(NP_073736) c3(protein-cysteine N-palmitoyltransferase porcupine isoform A) c4(213319, 213320) c5(213321, 213322, 213323, 213324, 213325, 213326, 213327, 213328, 213329, 213330) c6( ) c7(qC, av, T, afH, aW, aT, h, avF, aj, zV, arx, bHi, bz, J); #c1(PORCN) c2(NP_982299) c3(protein-cysteine N-palmitoyltransferase porcupine isoform B) c4(263331, 263332) c5(263333, 263334, 263335, 263336, 263337) c6( ) c7(qC, av, T, afH, aW, aT, h, avF, aj, zV, arx, bHi, bz, J); #c1(PORCN) c2(NP_982300) c3(protein-cysteine N-palmitoyltransferase porcupine isoform C) c4(263338) c5(263339, 263340, 263341, 263342, 263343, 263344, 263345, 263346, 263347, 263348, 263349) c6( ) c7(qC, av, T, afH, aW, aT, h, avF, aj, zV, arx, bHi, bz, J); #c1(PORCN) c2(NP_982301) c3 (protein-cysteine N-palmitoyltransferase porcupine isoform D) c4(263350) c5(263351, 263352, 263353, 263354, 263355, 263356, 263357, 263358, 263359, 263360, 263361) c6( ) c7(qC, av, T, afH, aW, aT, h, avF, aj, zV, arx, bHi, bz, J); #c1(PORCN) c2(XP_005272692) c3 (protein-cysteine N-palmitoyltransferase porcupine isoform X3) c4( ) c5(284149) c6( ) c7(qC, av, T, afH, aW, aT, h, avF, aj, zV, arx, bHi, bz, J); #c1(PORCN) c2(XP_005272693) c3(protein-cysteine N-palmitoyltransferase porcupine isoform X4) c4( ) c5(284150) c6( ) c7(qC, av, T, afH, aW, aT, h, avF, aj, zV, arx, bHi, bz, J); #c1(PORCN) c2(XP_005272694) c3(protein-cysteine N-palmitoyltransferase porcupine isoform X5) c4( ) c5(284151) c6( ) c7(qC, av, T, afH, aW, aT, h, avF, aj, zV, arx, bHi, bz, J); #c1(PORCN) c2(XP_006724607) c3(protein-cysteine N-palmitoyltransferase porcupine isoform X6) c4( ) c5(296108, 296109, 296110, 296111) c6( ) c7(qC, av, T, afH, aW, aT, h, avF, aj, zV, arx, bHi, bz, J); #c1(PORCN) c2(XP_006724608) c3(protein-cysteine N-palmitoyltransferase porcupine isoform X7) c4( ) c5(296112) c6( ) c7(qC, av, T, afH, aW, aT, h, avF, aj, zV, arx, bHi, bz, J); #c1(POSTN) c2(NP_001129406) c3 (periostin isoform 2 precursor) c4(51445) c5(51446, 51447, 51448, 51449, 51450, 51451, 51452) c6( ) c7(T, dD, aC, ea, aw, aA, c, eq, ci, o, bke, fS, ctR, aT, h, dL, eL, af, hm, aW, Y, uY, bz, es, iA, nB, Tu, X); #c1(POSTN) c2(NP_001129407) c3(periostin isoform 3 precursor) c4(51453) c5(51454, 51455, 51456, 51457, 51458, 51459, 51460) c6( ) c7(T, dD, aC, ea, aw, aA, cl, eq, ci, o, bke, fS, ctR, aT, h, dL, eL, af, hm, aW, Y, uY, bz, es, iA, nB, Tu, X); #c1(POSTN) c2(NP_001129408) c3(periostin isoform 4 precursor) c4( ) c5(51461) c6( ) c7(T, dD, aC, ea, aw, aA, cl, eq, ci, o, bke, fS, ctR, aT, h, dL, eL, af, hm, aW, Y, uY, bz, es, iA, nB, Tu, X); #c1(POSTN) c2(NP_001273594) c3(periostin isoform 5 precursor) c4(105386) c5(105387, 105388, 105389, 105390, 105391, 105392) c6( ) c7(T, dD, aC, ea, aw, aA, cl, eq, ci, o, bke, fS, ctR, aT, h, dL, eL, af, hm, aW, Y, uY, bz, es, iA, nB, Tu, X); #c1(POSTN) c2(NP_001273595) c3(periostin isoform 6 precursor) c4(105393) c5(105394, 105395, 105396, 105397, 105398, 105399) c6( ) c7(T, dD, aC, ea, aw, aA, cl, eq, ci, o, bke, fS, ctR, aT, h, dL, eL, af, hm, aW, Y, uY, bz, es, iA, nB, Tu, X); #c1(POSTN) c2(NP_001273596) c3(periostin isoform 7 precursor) c4( ) c5(105400, 105401, 105402, 105403, 105404) c6( ) c7(T, dD, aC, ea, aw, aA, cL, eq, ci, o, bke, fS, ctR, aT, h, dL, eL, af, hm, aW, Y, uY, bz, es, iA, nB, Tu, X); #c1(POSTN) c2(NP_006466) c3(periostin isoform 1 precursor) c4(165408) c5(165409, 165410, 165411, 165412, 165413, 165414, 165415, 165416) c6( ) c7(T, dD, aC, ea, aw, aA, cl, eq, ci, o, bke, fS, ctR, aT, h, dL, eL, af, hm, aW, Y, uY, bz, es, iA, nB, Tu, X); #c1(POSTN) c2(XP_005266288) c3(periostin isoform X1) c4(279956) c5(279957, 279958, 279959, 279960, 279961, 279962) c6( ) c7(T, dD, aC, ea, aw, aA, cl, eq, ci, o, bke, fS, ctR, aT, h, dL, eL, af, hm, aW, Y, uY, bz, es, iA, nB, Tu, X); #c1(POSTN) c2(XP_005266289) c3 (periostin isoform X2) c4(279963) c5( ) c6( ) c7(T, dD, aC, ea, aw, aA, cl, eq, ci, o, bke, fS, ctR, aT, h, dL, eL, af, hm, aW, Y, uY, bz, es, iA, nB, Tu, X); #c1(POT1) c2(XP_006715980) c3(protection of telomeres protein 1 isoform X2) c4(289958, 289959, 289960, 289961, 289962, 289963, 289964) c5(289965, 289966, 289967, 289968, 289969, 289970, 289971, 289972, 289973, 289974, 289975, 289976, 289977, 289978, 289979, 289980, 289981, 289982, 289983, 289984, 289985, 289986, 289987, 289988, 289989, 289990, 289991, 289992, 289993, 289994, 289995, 289996, 289997, 289998, 289999, 290000, 290001) c6( ) c7(sc, T, yk, e, jh, aA, n, Fc, auf, aT, kH, hn, dL, je, aW, Y, ff, r, aF, en, bz, sS, iM, ac, hk, hl, cj, at, iS, B); #c1(POT1) c2(XP_011514309) c3(protection of telomeres protein 1 isoform X1) c4( ) c5(306994, 306995, 306996, 306997, 306998) c6( ) c7(sc, T, yk, e, jh, aA, n, Fc, auf, aT, kH, hn, dL, je, aW, Y, ff, r, aF, en, bz, sS, iM, ac, hk, hl, cj, at, iS, B); #c1(POTED) c2(NP_778146) c3(POTE ankyrin domain family member D) c4(251361) c5(251362, 251363, 251364, 251365, 251366, 251367) c6( ) c7(T, aW, aT, m, bu, t, ae, ad); #c1(POTEF) c2(NP_001093241) c3(POTE ankyrin domain family member F) c4(40122, 40123, 40124) c5(40125, 40126, 40127, 40128, 40129, 40130, 40131, 40132, 40133, 40134, 40135, 40136, 40137, 40138, 40139, 40140, 40141, 40142, 40143, 40144, 40145, 40146) c6( ) c7(bM, T, pf, al, bB, ci, zW, ip, x, aE, aU, eL, li, AP, aW, g, as, bw, eM, AM, vA, mx, iS, k, wu, nA, eW, tD, co, ft, h, dL, ey, AT, AQ, fe, op, iO, bP, sZ, AN, B, AB, fM, dA, AL, AS, aA, v, j, AE, AD, q, eJ, AK, aT, kf, AJ, bK, ae, kl, s, iU, Y, r, lo, en, f, AA, AH, hr, AU, AC, gT, bt, vi, yv, jh, Ay, ad, dP, AD, ds, qh, ab, ji, wm, AF, ff, aF, bz, dr, bF, gk, bE, AR, gZ, bC, ei, hE, AG); #c1(POTEG) c2(NP_001005356) c3(POTE ankyrin domain family member G) c4(21448) c5(21449, 21450, 21451, 21452, 21453, 21454) c6( ) c7(T); #c1(POTEH) c2(NP_001129685) c3(POTE ankyrin domain family member H) c4(52194, 52195) c5(52196, 52197, 52198, 52199, 52200, 52201, 52202, 52203, 52204, 52205, 52206, 52207) c6( ) c7(T, eA); #c1(POTEM) c2(NP_001138914) c3(putative POTE ankyrin domain family member M) c4(58145, 58146, 58147, 58148) c5(58149, 58150, 58151, 58152, 58153, 58154, 58155, 58156, 58157, 58158, 58159, 58160, 58161, 58162, 58163, 58164, 58165, 58166, 58167, 58168, 58169, 58170, 58171, 58172) c6( ) c7(bL, bM, T, aw, ct, eA, qH, AW, vJ, sO, il, gd, aT, dV, iv, bK, an, af, A, aW, AV, at, eg); #c1(POU1F1) c2(NP_000297) c3(pituitary-specific positive transcription factor 1 isoform alpha) c4(6700) c5(6701, 6702, 6703, 6704, 6705, 6706) c6( ) c7(bBJ, Ly, lT, acz, hz, bBS, ci, nA, AO, ad, biN, fU, ib, oW, aT, aQy, yo, avP, wr, s, aW, CM, bBO, wU, ctS, OG, hR, lS, im, gT, VK, aya); #c1(POU1F1) c2(NP_001116229) c3(pituitary-specific positive transcription factor 1 isoform beta) c4(43692) c5(43693, 43694, 43695, 43696, 43697, 43698) c6( ) c7(bBJ, Ly, lT, acz, hz, bBS, ci, nA, AO, ad, biN, fU, ib, oW, aT, aQy, yo, avP, wr, s, aW, CM, bBO, wU, ctS, OG, hR, lS, im, gT, VK, aya); #c1(POU2AF1) c2(NP_006226) c3(POU domain class 2-associating factor 1) c4(163113) c5(163114, 163115, 163116, 163117, 163118, 163119) c6( ) c7(T, fM, wu, ex, jh, fd, hF, V, cd, pe, h, eV, wm, bc, Of, eZ, r, Og, zL, Wt, cj, bt, Ze); #c1(POU2AF1) c2(XP_006718922) c3(POU domain class 2-associating factor 1 isoform X2) c4( ) c5(292112) c6( ) c7(T, fM, wu, ex, jh, fd, hF, V, cd, pe, h, eV, wm, bc, Of, eL, r, Og, zL, Wt, cj, bt, Ze); #c1(POU2F1) c2(NP_001185712) c3(POU domain, class 2, transcription factor 1 isoform 2) c4(76096) c5(76097, 76098, 76099, 76100, 76101, 76102, 76103, 76104, 76105, 76106, 76107, 76108, 76109, 76110) c6( ) c7(bL, dD, fM, bkx, ea, if, ht, jh, aA, cz, v, eq, ci, eh, Hr, jz, F, Do, aMb, qp, dV, aT, K, hu, af, bc, bA, d, iU, Of, fe, Og, aF, hJ, eM, eD, hR, y, B); #c1(POU2F1) c2(NP_001185715) c3(POU domain, class 2, transcription factor 1 isoform 3) c4(76111, 76112) c5(76113, 76114, 76115, 76116, 76117, 76118, 76119) c6( ) c7(bL, dD, fM, bkx, ea, if, ht, jh, aA, cz, v, eq, ci, eh, Hr, jz, F, Do, aMb, qp, dV, aT, K, hu, af, bc, bA, d, iU, Of, fe, Og, aF, hJ, eM, eD, hR, y, B); #c1(POU2F1) c2(NP_002688) c3(POU domain, class 2, transcription factor 1 isoform 1) c4(130944, 130945, 130946, 130947) c5( ) c6( ) c7(bL, dD, fM, bkx, ea, if, ht, jh, aA, cz, v, eq, ci, eh, Hr, jz, F, Do, aMb, qp, dV, aT, K, hu, af, bc, bA, d, iU, Of, fe, Og, aF, hJ, eM, eD, hR, y, B); #c1(POU2F1) c2(XP_011507955) c3(POU domain, class 2, transcription factor 1 isoform X1) c4(298315, 298316, 298317) c5( ) c6( ) c7(bL, dD, fM, bkx, ea, if, ht, jh, aA, cz, v, eq, ci, eh, Hr, jz, F, Do, aMb, qp, dV, aT, K, hu, af, bc, bA, d, iU, Of, fe, Og, aF, hJ, eM, eD, hR, y, B); #c1(POU2F1) c2(XP_011507956) c3(POU domain, class 2, transcription factor 1 isoform X2) c4(298318) c5( ) c6( ) c7(bL, dD, fM, bkx, ea, if, ht, jh, aA, cz, v, eq, ci, eh, Hr, jz, F, Do, aMb, qp, dV, aT, K, hu, af, bc, bA, d, iU, Of, fe, Og, aF, hJ, eM, eD, hR, y, B); #c1(POU2F1) c2(XP_011507957) c3(POU domain, class 2, transcription factor 1 isoform X3) c4(298319) c5(298320, 298321, 298322, 298323, 298324, 298325, 298326, 298327, 298328, 298329, 298330, 298331, 298332, 298333, 298334, 298335, 298336, 298337, 298338, 298339, 298340, 298341, 298342, 298343, 298344, 298345, 298346, 298347, 298348, 298349, 298350, 298351, 298352, 298353, 298354, 298355, 298356, 298357, 298358, 298359, 298360, 298361, 298362, 298363, 298364, 298365, 298366, 298367, 298368, 298369, 298370, 298371, 298372, 298373, 298374, 298375, 298376, 298377) c6( ) c7(bL, dD, fM, bkx, ea, if, ht, jh, aA, cz, v, eq, ci, eh, Hr, jz, F, Do, aMb, qp, dV, aT, K, hu, af, bc, bA, d, iU, Of, fe, Og, aF, hJ, eM, eD, hR, y, B); #c1(POU2F2) c2(NP_001193954) c3(POU domain, class 2, transcription factor 2 isoform 1) c4(83329, 83330) c5(83331, 83332, 83333, 83334, 83335, 83336, 83337, 83338, 83339, 83340, 83341, 83342) c6( ) c7(T, fM, if, eX, RC, aA, fd, jh, bX, qN, q, er, bv, aE, aT, KH, eV, hB, pk, nJ, aW, Y, Of, Og, aF, zL, ck, dy, bt, B); #c1(POU2F2) c2(NP_001193955) c3(POU domain, class 2, transcription factor 2 isoform 3) c4(83343) c5(83344, 83345, 83346, 83347, 83348, 83349) c6( ) c7(T, fM, if, ex, RC, aA, fd, jh, bX, qN, q, er, bv, aE, aT, kH, eV, hB, pk, nJ, aW, Y, Of, Og, aF, zL, ck, dy, bt, B); #c1(POU2F2) c2(NP_001234923) c3(POU domain, class 2, transcription factor 2 isoform 4) c4(87774) c5(87775, 87776, 87777, 87778, 87779, 87780, 87781) c6( ) c7(T, fM, if, eX, RC, aA, fd, jh, bX, qN, q, er, bv, aE, aT, kH, eV, hB, pk, nJ, aW, Y, Of, Og, aF, zL, ck, dy, bt, B); #c1(POU2F2) c2(NP_002689) c3(POU domain, class 2, transcription factor 2 isoform 2) c4(130948) c5(130949, 130950, 130951, 130952, 130953, 130954) c6( ) c7(T, fM, if, eX, RC, aA, fd, jh, bX, qN, q, er, bv, aE, aT, kH, eV, hB, pk, nJ, aW, Y, Of, Og, aF, zL, ck, dy, bt, B); #c1(POU2F2) c2(XP_005259067) c3(POU domain, class 2, transcription factor 2 isoform X2) c4(275941) c5(275942, 275943, 275944, 275945, 275946, 275947) c6( ) c7(T, fM, if, eX, RC, aA, fd, jh, bX, qN, q, er, bv, aE, aT, kH, eV, hB, pk, nJ, aW, Y, Of, Og, aF, zL, ck, dy, bt, B); #c1(POU2F2) c2(XP_011525342) c3(POU domain, class 2, transcription factor 2 isoform X1) c4(319805) c5(319806, 319807, 319808, 319809, 319810, 319811) c6( ) c7(T, fM, if, eX, RC, aA, fd, jh, bX, qN, q, er, bv, aE, aT, kH, eV, hB, pk, nJ, aW, Y, Of, Og, aF, zL, ck, dy, bt, B); #c1(POU2F3) c2(NP_001231611) c3(POU domain, class 2, transcription factor 3 isoform 2) c4(87353) c5(87354, 87355, 87356, 87357, 87358, 87359) c6( ) c7(P, bL, dD, ea, if, e, aw, aA, bLo, j, eq, qN, aT, hH, cbb, arl, aW, aF, kT, bTF, ac, te, aS, Pr, abW); #c1(POU2F3) c2(NP_055167) c3(POU domain, class 2, transcription factor 3 isoform 1) c4(179563) c5(179564, 179565, 179566, 179567, 179568, (79569) c6( ) c7(P, bL, dD, ea, if, e, aw, aA, bLo, j, eq, qN, aT, hH, cbb, arl, aW, aF, kT, bTF, ac, te, aS, Pr, abW); #c1(POU3F2) c2(NP_005595) c3(POU domain, class 3, transcription factor 2) c4(157771, 157772) c5(157773, 157774, 157775, 157776, 157777, 157778, 157779, 157780, 157781, 157782, 157783, 157784, 157785) c6( ) c7(av, T, ft, kj, dA, Y, bK, rz); #c1(POU3F3) c2(NP_006227) c3(POU domain, class 3, transcription factor 3) c4(163120) c5(163121, 163122, 163123, 163124, 163125, 163126, 163127, 163128) c6( ) c7(it, nc); #c1(POU3F4) c2(NP_000298) c3(POU domain, class 3, transcription factor 4) c4(6707) c5(6708, 6709, 6710, 6711, 6712, 6713) c6( ) c7(AT, ckN, mG, nd, aMT, ctT, fr, nA, Bh); #c1(POU4F1) c2(NP_006228) c3(POU domain, class 4, transcription factor 1) c4(163129) c5(163130, 163131, 163132, 163133, 163134, 163135, 163136) c6( ) c7(cZ, T, ya, if, oH, qN, OH, ad, bv, lg, akU, pX, K, dd, ae, jf, Y, eZ, cm, ck, ctU, hj, bt, B); #c1(POU4F2) c2(NP_004566) c3(POU domain, class 4, transcription factor 2) c4(147807) c5(147808, 147809, 147810, 147811, 147812, 147813, 147814) c6( ) c7(cZ, T, ctU, lg, aW, ahL, eZ, aT, n, dd, cm); #c1(POU4F3) c2(NP_002691) c3(POU domain, class 4, transcription factor 3) c4(130955) c5(130956, 130957, 130958, 130959, 130960, 130961) c6( ) c7(mG, EQ, aOH, K, ctV, Bh); #c1(POU5F1) c2(NP_001272915) c3(POU domain, class 5, transcription factor 1 isoform 4) c4( ) c5(104093, 104094, 104095, 104096, 104097, 104098, 104099, 104100) c6( ) c7(ID, xS, T, aiH, if, ci, aE, il, aWi, PI, K, MO, hH, eL, xa, ly, DA, xQ, UL, ZH, aW, aeC, g, hJ, eF, cm, wT, eM, m, gn, QO, wu, iB, aw, n, ctW, qN, BI, jz, aoc, TK, dL, CN, AQ, Lh, zZ, Ld, lx, eD, it, aAE, B, aA, lu, V, ctX, q, eA, ns, yg, aT, rO, ae, vT, Y, Lf, r, en, bu, t, ik, ck, QG, Da, KY, bok, aD, jh, Hn, ad, BY, dP, vJ, wm, nJ, mu, d, lx, ff, eH, cr, eZ, aF, bz, Lb, lC, iu); #c1(POU5F1) c2(NP_001272916) c3(POU domain, class 5, transcription factor 1 isoform 3) c4(104101, 104102) c5(104103, 104104, 104105, 104106, 104107, 104108, 104109, 104110, 104111, 104112, 104113, 104114, 104115, 104116, 104117, 104118, 104119, 104120, 104121, 104122, 104123, 104124, 104125, 104126) c6( ) c7(ID, xS, T, aiH, if, ci, aE, il, aWi, PI, K, MO, hH, eL, xa, ly, DA, xQ, UL, ZH, aW, aeC, g, hJ, eF, cm, wT, eM, m, gn, QO, wu, iB, aw, n, ctW, qN, BI, jz, aoc, TK, dL, CN, AD, Lh, zZ, Ld, lx, eD, it, aAE, B, aA, lu, V, ctX, q, eA, ns, yg, al, rO, ae, vT, Y, Lf, r, en, bu, t, ik, ck, QG, Da, KY, bok, aD, jh, Hn, ad, BY, dP, vJ, wm, nJ, mu, d, lx, ff, eH, cr, eZ, aF, bz, Lb, lC, iu); #c1(POU5F1) c2(NP_002692) c3(POU domain, class 5, transcription factor 1 isoform 1) c4(130962, 130963, 130964, 130965) c5(130966, 130967, 130968, 130969, 130970, 130971, 130972, 130973, 130974, 130975, 130976, 130977, 130978, 130979, 130980, 130981, 130982, 130983, 130984, 130985, 130986, 130987, 130988, 130989, 130990, 130991, 130992, 130993, 130994, 130995, 130996, 130997, 130998, 130999, 131000, 131001, 131002, 131003, 131004, 131005, 131006, 131007, 131008, 131009, 131010, 131011, 131012, 131013, 131014, 131015, 131016, 131017, 131018, 131019, 131020, 131021, 131022, 131023, 131024, 131025, 131026, 131027, 131028, 131029, 131030, 131031, 131032, 131033, 131034, 131035, 131036, 131037, 131038, 131039, 131040, 131041, 131042, 131043, 131044, 131045, 131046, 131047, 131048, 131049, 131050, 131051, 131052, 131053, 131054, 131055, 131056, 131057, 131058, 131059, 131060, 131061, 131062, 131063, 131064, 131065, 131066, 131067, 131068, 131069, 131070, 131071, 131072, 131073, 131074, 131075, 131076, 131077, 131078, 131079, 131080, 131081, 131082, 131083, 131084, 131085, 131086, 131087, 131088, 131089, 131090, 131091, 131092, 131093, 131094, 131095, 131096, 131097, 131098, 131099, 131100, 131101, 131102, 131103, 131104, 131105, 131106, 131107, 131108, 131109, 131110, 131111, 131112, 131113, 131114, 131115, 131116, 131117, 131118, 131119, 131120, 131121, 131122, 131123, 131124, 131125, 131126, 131127, 131128, 131129, 131130, 131131, 131132, 131133, 131134, 131135, 131136, 131137, 131138, 131139, 131140, 131141) c6(131142) c7(ID, xS, T, aiH, if, ci, aE, iI, aWi, PI, K, MO, hH, eL, xa, ly, DA, xQ, UL, ZH, aW, aeC, g, hJ, eF, cm, wT, eM, m, gn, QO, wu, iB, aw, n, ctW, qN, BI, jz, aoc, TK, dL, CN, AQ, Lh, zZ, Ld, lx, eD, it, aAE, B, aA, lu, V, ctX, q, eA, ns, yg, aT, rO, ae, vT, Y, Lf, r, en, bu, t, ik, ck, QG, Da, KY, bok, aD, jh, Hn, ad, BY, dP, vJ, wm, nJ, mu, d, lx, ff, eH, cr, eZ, aF, bz, Lb, lC, iu); #c1(POU5F1B) c2(NP_001153014) c3(putative POU domain, class 5, transcription factor (B) c4(60317) c5(60318, 60319, 60320, 60321, 60322, 60323, 60324, 60325, 60326, 60327, 60328) c6( ) c7(T, dL, ci, t, ae, ad); #c1(POU6F1) c2(NP_002693) c3(POU domain, class 6, transcription factor 1) c4(131143, 131144) c5(131145, 131146, 131147, 131148, 131149, 131150, 131151, 131152, 131153, 131154, 131155, 131156, 131157, 131158, 131159, 131160, 131161, 131162, 131163, 131164, 131165) c6( ) c7(aqt, aqp, Z, Ei, ctY, gf, bS, K, ct, nA, xh, he, bk, aR); #c1(POU6F2) c2(NP_001159490) c3(POU domain, class 6, transcription factor 2 isoform 2) c4(64162) c5(64163, 64164, 64165, 64166, 64167, 64168, 64169) c6( ) c7(er, an, bN, ctZ); #c1(POU6F2) c2(NP_009183) c3(POU domain, class 6, transcription factor 2 isoform 1) c4(171348) c5( ) c6( ) c7(er, an, bN, ctZ); #c1(POU6F2) c2(XP_011513416) c3(POU domain, class 6, transcription factor 2 isoform X2) c4( ) c5(305653, 305654, 305655, 305656, 305657, 305658) c6( ) c7(er, an, bN, ctZ); #c1(PPAI) c2(NP_066952) c3(inorganic pyrophosphatase) c4(207671, 207672) c5(207673, 207674, 207675, 207676, 207677, 207678, 207679, 207680, 207681, 207682, 207683, 207684) c6( ) c7(WY, aaE, T, cd, g); #c1(PPA2) c2(NP_008834) c3(inorganic pyrophosphatase 2, mitochondrial isoform 2 precursor) c4(168931) c5(168932, 168933, 168934, 168935, 168936, 168937) c6( ) c7(aoz); #c1(PPA2) c2(NP_789842) c3(inorganic pyrophosphatase 2, mitochondrial isoform 3 precursor) c4(252359) c5(252360, 252361, 252362, 252363, 252364, 252365) c6( ) c7(aoz); #c1(PPA2) c2(NP_789843) c3(inorganic pyrophosphatase 2, mitochondrial isoform 4 precursor) c4(252366) c5(252367, 252368, 252369, 252370, 252371, 252372) c6( ) c7(aoz); #c1(PPA2) c2(NP_789845) c3(inorganic pyrophosphatase 2, mitochondrial isoform 1 precursor) c4(252373, 252374, 252375, 252376) c5(252377, 252378, 252379, 252380, 252381, 252382, 252383, 252384, 252385, 252386, 252387, 252388, 252389, 252390, 252391, 252392, 252393, 252394, 252395, 252396, 252397, 252398, 252399, 252400) c6( ) c7(aoz); #c1(PPAP2A) c2(NP_003702) c3(lipid phosphate phosphohydrolase 1 isoform 1) c4(140266) c5(140267, 140268, 140269, 140270, 140271, 140272) c6( ) c7(t, Fb, aA); #c1(PPAP2A) c2(NP_795714) c3(lipid phosphate phosphohydrolase 1 isoform 2) c4(252469) c5(252470, 252471, 252472, 252473, 252474, 252475) c6( ) c7(t, Fb, aA); #c1(PPAP2C) c2(NP_003703) c3(lipid phosphate phosphohydrolase 2 isoform 1) c4(140273) c5(140274, 140275, 140276, 140277, 140278, 140279) c6( ) c7(T, ci); #c1(PPAP2C) c2(NP_803545) c3(lipid phosphate phosphohydrolase 2 isoform 2) c4(252658) c5(252659, 252660, 252661, 252662, 252663) c6( ) c7(T, ci); #c1(PPAP2C) c2(NP_808211) c3(lipid phosphate phosphohydrolase 2 isoform 3) c4(252703) c5(252704, 252705, 252706, 252707, 252708, 252709, 252710) c6( ) c7(T, ci); #c1(PPAPDC1B) c2(NP_001096029) c3(phosphatidate phosphatase PPAPDC1B isoform 1) c4(40968) c5(40969, 40970, 40971, 40972, 40973, 40974) c6( ) c7(av, ft, eJ, aW, aT, n, bz, B); #c1(PPAPDC1B) c2(NP_001096030) c3(phosphatidate phosphatase PPAPDC1B isoform 3) c4(40975) c5(40976, 40977, 40978, 40979, 40980, 40981) c6( ) c7(av, ft, eJ, aW, aT, n, bz, B); #c1(PPAPDC1B) c2(NP_115872) c3 (phosphatidate phosphatase PPAPDC1B isoform 2) c4(224418) c5(224419, 224420, 224421, 224422, 224423, 224424) c6( ) c7(av, ft, eJ, aW, aT, n, bz, B); #c1(PPAPDC2) c2(NP_982278) c3(presqualene diphosphate phosphatase) c4(263265, 263266) c5(263267, 263268, 263269, 263270, 263271, 263272, 263273, 263274, 263275, 263276, 263277, 263278) c6( ) c7(ck, T); #c1(PPAPDC3) c2(NP_116117) c3(probable lipid phosphate phosphatase PPAPDC3) c4(225399, 225400) c5(225401, 225402, 225403, 225404, 225405, 225406) c6( ) c7(cd); #c1(PPAPDC3) c2(XP_005272288) c3(probable lipid phosphate phosphatase PPAPDC3 isoform X2) c4( ) c5(283986, 283987, 283988, 283989, 283990, 283991) c6( ) c7(cd); #c1(PPARA) c2(XP_011528545) c3(peroxisome proliferator-activated receptor alpha isoform X1) c4(323673, 323674, 323675, 323676) c5(323677, 323678, 323679, 323680, 323681, 323682, 323683, 323684, 323685, 323686, 323687, 323688, 323689, 323690, 323691, 323692, 323693, 323694, 323695, 323696, 323697, 323698, 323699, 323700, 323701, 323702, 323703, 323704, 323705, 323706, 323707, 323708, 323709, 323710, 323711, 323712, 323713, 323714, 323715, 323716, 323717) c6( ) c7(bM, T, FR, fV, ea, e, al, if, l, bB, ci, ip, GK, bv, Bc, M, K, qM, wt, da, dF, eN, zQ, aW, fc, hJ, ve, uC, eE, GR, cm, eM, iA, wK, mx, cj, ym, dD, aw, cl, aj, fr, qN, zP, cd, cc, lG, jy, hn, dL, cgO, wC, fJ, AQ, ks, aq, iO, cO, Fi, bo, mg, at, uz, B, bL, ul, eR, oe, jx, qQ, cz, v, j, so, sl, nS, V, q, J, mP, aql, aT, cy, bq, sR, ae, af, eu, bA, kl, Y, r, iy, lo, uU, en, ck, kP, ac, hc, mf, dy, bTg, cuc, ao, Mb, sY, id, ow, aZ, aET, dG, eq, Fb, ad, hX, dP, cua, av, oz, oj, ji, lQ, d, cH, cW, ff, cr, dN, cub, dq, gu, iV, iR, bz, DF, es, bE, gZ, ME, cP, ei); #c1(PPARA) c2(XP_011528547) c3(peroxisome proliferator-activated receptor alpha isoform X2) c4(323718) c5(323719, 323720, 323721, 323722, 323723, 323724) c6( ) c7(bM, T, FR, fV, ea, e, al, if, l, bB, ci, ip, GK, bv, Bc, M, K, qM, wt, dQ, dF, eN, zQ, aW, fc, hJ, ve, uC, eE, GR, cm, eM, iA, wK, mx, cj, ym, dD, aw, cl, aj, fr, qN, zP, cd, cc, lG, jy, hn, dL, cgO, wC, fJ, AQ, ks, aq, iO, cO, Fi, bo, mg, at, uz, B, bL, ul, eR, oe, jx, qQ, cz, v, j, so, sl, nS, V, q, J, mP, aql, aT, cy, bq, sR, ae, af, eu, bA, kl, Y, r, iy, lo, ul, en, ck, kP, ac, hc, mf, dy, bTg, cuc, ao, Mb, sY, id, ow, aZ, aET, dG, eq, Fb, ad, hX, dP, cua, av, oz, oj, ji, lQ, d, cH, cW, ff, cr, dN, cub, dq, gu, iV, iR, bz, DF, es, bE, gZ, ME, cP, ei); #c1(PPARD) c2(NP_001165290) c3(peroxisome proliferator-activated receptor delta isoform 3) c4(67591) c5(67592, 67593, 67594, 67595, 67596, 67597, 67598, 67599, 67600, 67601, 67602, 67603) c6( ) c7(bL, T, Ve, dD, eR, ea, yk, e, NT, aA, cz, bB, aj, v, j, ci, bX, eq, mF, q, J, cd, ad, av, jz, cc, Bc, NS, aeO, M, aT, h, dL, bK, K, lQ, bq, dQ, ae, af, kl, B, aeR, s, JI, aW, ff, az, hl, cr, r, nY, dq, fc, aF, en, g, cm, bz, bJ, bF, bP, eM, nB, bE, hk, AM, mf, m, cP, ei, adg, xb, X); #c1(PPARD) c2(NP_001165291) c3(peroxisome proliferator-activated receptor delta isoform 4) c4(67604) c5(67605, 67606, 67607, 67608, 67609, 67610) c6( ) c7(bL, T, Ve, dD, eR, ea, yk, e, NT, aA, cz, bB, aj, v, j, ci, bX, eq, mF, q, J, cd, ad, av, jz, cc, Bc, NS, aeO, M, aT, h, dL, bK, K, lQ, bq, dQ, ae, af, kl, B, aeR, s, JI, aW, ff, az, hl, cr, r, nY, dq, fc, aF, en, g, cm, bz, bJ, bF, bP, eM, nB, bE, hk, AM, mf, m, cP, ei, adg, xb, X); #c1(PPARD) c2(NP_803184) c3(peroxisome proliferator-activated receptor delta isoform 2) c4(252536) c5(252537, 252538, 252539, 252540, 252541, 252542, 252543, 252544, 252545, 252546) c6( ) c7(bL, T, Ve, dD, eR, ea, yk, e, NT, aA, cz, bB, aj, v, j, ci, bX, eq, mF, q, J, cd, ad, av, jz, cc, Bc, NS, aeO, M, aT, h, dL, bK, K, lQ, bq, dQ, ae, af, kl, B, aeR, s, JI, aW, ff, az, hl, cr, r, nY, dq, fc, aF, en, g, cm, bz, bJ, bF, bP, eM, nB, bE, hk, AM, mf, m, cP, ei, adg, xb, X); #c1(PPARD) c2(XP_006715186) c3(peroxisome proliferator-activated receptor delta isoform X1) c4(288812, 288813) c5(288814, 288815, 288816, 288817, 288818, 288819, 288820, 288821, 288822, 288823, 288824) c6( ) c7(bL, T, Ve, dD, eR, ea, yk, e, NT, aA, cz, bB, aj, v, j, ci, bX, eq, mF, q, J, cd, ad, av, jz, cc, Bc, NS, aeO, M, aT, h, dL, bk, K, lQ, bq, dQ, ae, af, kl, B, aeR, s, JI, aW, ff, az, hl, cr, r, nY, dq, fc, aF, en, g, cm, bz, bJ, bF, bP, eM, nB, bE, hk, AM, mf, m, cP, ei, adg, xb, X); #c1(PPARG) c2(NP_005028) c3 (peroxisome proliferator-activated receptor gamma isoform 1) c4(152280, 152281, 152282) c5(152283, 152284, 152285, 152286, 152287, 152288, 152289, 152290, 152291, 152292, 152293, 152294, 152295, 152296) c6( ) c7(ID, gh, T, bM, Ap, aC, fR, pf, YU, ea, if, e, al, fd, bB, ci, aTS, aon, mF, aR, ku, wD, dY, u, aE, iI, Fo, M, dV, cpD, K, qM, hu, dQ, H, eL, bc, eN, hm, aa, ly, yo, JI, aW, az, hl, LZ, nY, fc, vs, aB, g, hJ, ve, uC, eE, Vm, GR, eF, cm, qJ, bw, fT, bXI, eM, nB, wK, vA, mx, U, vy, uJ, nw, dW, m, lS, du, hh, jM, ym, OD, iu, FW, dD, vl, lT, wh, aw, ht, NT, n, BH, aj, afE, fr, qN, JW, cd, ai, acy, TB, td, ke, jz, Wy, hw, aoc, oN, Nn, cU, lG, jy, dL, ey, h, aAG, uV, bl, Xh, eV, cue, pi, bXD, wC, hi, A, fJ, iw, ge, fQ, aq, fe, kp, zZ, cM, op, lx, JP, jq, bJ, kT, JR, GG, ar, Qy, bP, cO, dB, Fi, mg, hj, hL, E, uz, hR, at, bm, aMI, B, AN, L, bL, Dw, jv, fM, dA, AS, eR, fG, De, eX, aA, cR, GP, cz, v, j, C, nS, V, GS, q, J, eA, Kx, GL, mP, yw, uT, lg, nX, aNA, MP, HC, aT, cy, Fx, acH, yb, bq, an, j, ae, af, eu, bA, jf, VC, kl, wr, bf, s, BA, iU, oV, Y, ev, r, iy, en, hf, bSy, t, cl, pb, ck, rR, nM, kP, ua, c, ac, hc, au, dh, gX, hr, dT, JL, mf, cS, dy, fD, bt, xb, ao, kV, vq, sY, kh, bPo, Ke, akd, aZ, aD, df, iW, jh, dG, eq, jE, OH, gb, tK, aue, ad, kL, hX, dP, fU, av, l, LL, hd, ib, Db, oz, py, Ot, ds, NS, lC, oj, QJ, hD, vc, vB, ji, lQ, jo, nJ, sj, ul, uX, d, nU, cH, PE, ff, eH, zR, cr, Mz, cud, dq, gE, xv, aF, eZ, iV, Z, iR, KM, bz, qa, ek, bF, es, D, NC, mi, yE, bE, ag, z, eB, aam, gZ, aMy, aS, Kj, cP, ei, Zw, kS, X, y, acG); #c1(PPARG) c2(NP_056953) c3(peroxisome proliferator-activated receptor gamma isoform 2) c4(188190, 188191, 188192, 188193) c5(188194, 188195, 188196, 188197, 188198, 188199, 188200, 188201, 188202, 188203, 188204, 188205, 188206, 188207, 188208, 188209, 188210, 188211) c6( ) c7(ID, gh, T, bM, Ap, aC, fR, pf, YU, ea, if, e, al, fd, bB, ci, aTS, aon, mF, aR, ku, wD, dY, u, aE, iI, Fo, M, dV, cpD, K, qM, hu, dQ, H, eL, bc, eN, hm, aa, ly, yo, JI, aW, az, hl, LZ, nY, fc, vs, aB, g, hJ, ve, uC, BE, Vm, GR, eF, cm, qJ, bw, fT, bXI, eM, nB, wK, vA, mx, U, vy, uJ, nw, dW, m, lS, du, hh, jM, ym, OD, iu, FW, dD, vl, lT, wh, aw, ht, NT, n, BH, aj, afE, fr, qN, JW, cd, ai, acy, TB, td, ke, jz, Wy, hw, aoc, ON, Nn, cU, lG, jy, dL, ey, h, aAG, uV, bl, Xh, eV, cue, pi, bXD, wC, hi, A, fJ, iw, ge, fQ, aq, fe, kp, zZ, cM, op, lx, JP, jq, bJ, kT, JR, GG, ar, Qy, bP, cO, dB, Fi, mg, hj, hL, E, uz, hR, at, bm, aMI, B, AN, L, bL, Dw, jv, fM, dA, AS, eR, fG, De, eX, aA, cR, GP, cz, v, j, C, nS, V, GS, q, J, eA, Kx, GL, mP, yw, uT, lg, nX, aNA, MP, HC, aT, cy, Fx, acH, yb, bq, an, j, ae, af, eu, bA, jf, VC, kl, wr, bf, s, BA, iU, oV, Y, ev, r, iy, en, hf, bSy, t, cl, pb, ck, rR, nM, kP, ua, c, ac, hc, au, dh, gX, hr, dT, JL, mf, cS, dy, fD, bt, xb, ao, kV, vq, sY, kh, bPo, Ke, akd, aZ, aD, df, iW, jh, dG, eq, jE, OH, gb, tK, aue, ad, kL, hX, dP, fU, av, l, LL, hd, ib, Db, oz, py, Ot, ds, NS, lC, oj, QJ, hD, vc, vB, ji, lQ, jo, nJ, sj, ul, uX, d, nU, cH, PE, ff, eH, zR, cr, Mz, cud, dq, gE, xv, aF, eZ, iV, Z, iR, KM, bz, qa, ek, bF, es, D, NC, mi, yE, bE, ag, z, eB, aam, gZ, aMy, aS, Kj, cP, ei, Zw, kS, X, y, acG); #c1(PPARG) c2(XP_011532144) c3 (peroxisome proliferator-activated receptor gamma isoform X2) c4( ) c5(328169) c6( ) c7(ID, gh, T, bM, Ap, aC, fR, pf, YU, ea, if, e, al, fd, bB, ci, aTS, aon, mF, aR, ku, wD, dY, u, aE, iI, Fo, M, dV, cpD, K, qM, hu, dQ, H, eL, bc, eN, hm, aa, ly, yo, JI, aW, az, hl, LZ, nY, fc, vs, aB, g, hJ, ve, uC, eE, Vm, GR, eF, cm, qJ, bw, fT, bXI, eM, nB, wK, vA, mx, U, vy, uJ, nw, dW, m, lS, du, hh, jM, ym, OD, iu, FW, dD, vl, lT, wh, aw, ht, NT, n, BH, aj, afE, fr, qN, JW, cd, ai, acy, TB, td, ke, jz, Wy, hw, aoc, oN, Nn, cU, lG, jy, dL, ey, h, aAG, uV, bl, Xh, eV, cue, pi, bXD, wC, hi, A, fJ, iw, ge, fQ, aq, fe, kp, zZ, cM, op, lx, JP, jq, bJ, kT, JR, GG, ar, Qy, bP, cO, dB, Fi, mg, hj, hL, E, uz, hR, at, bm, aMI, B, AN, L, bL, Dw, jv, fM, dA, AS, eR, fG, oe, eX, aA, cR, GP, cz, v, j, C, nS, V, GS, q, J, eA, Kx, GL, mP, yw, uT, lg, nX, aNA, MP, HC, aT, cy, Fx, acH, yb, bq, an, j, ae, af, eu, bA, jf, VC, kl, wr, bf, s, BA, iU, oV, Y, ev, r, iy, en, hf, bSy, t, cl, pb, ck, rR, nM, kP, ua, c, ac, hc, au, dh, gX, hr, dT, JL, mf, cS, dy, fD, bt, xb, ao, kV, vq, sY, kh, bPo, Ke, akd, aZ, aD, df, iW, jh, dG, eq, jE, OH, gb, tK, aue, ad, kL, hX, dP, fU, av, l, LL, hd, ib, Db, oz, py, Ot, ds, NS, lC, oj, QJ, hD, vc, vB, ji, lQ, jo, nJ, sj, ul, uX, d, nU, cH, PE, ff, eH, zR, cr, Mz, cud, dq, gE, xv, aF, eZ, iV, Z, iR, KM, bz, qa, ek, bF, es, D, NC, mi, yE, bE, ag, z, eB, aam, gZ, aMy, aS, Kj, cP, ei, Zw, kS, X, y, acG); #c1(PPARG) c2(XP_011532145) c3 (peroxisome proliferator-activated receptor gamma isoform X3) c4(328170, 328171) c5(328172, 328173, 328174) c6( ) c7(ID, gh, T, bM, Ap, aC, fR, pf, YU, ea, if, e, al, fd, bB, ci, aTS, aon, mF, aR, ku, wD, dY, u, aE, iI, Fo, M, dV, cpD, K, qM, hu, dQ, H, eL, bc, eN, hm, aa, ly, yo, JI, aW, az, hl, LZ, nY, fc, vs, aB, g, hJ, ve, uC, eE, Vm, GR, eF, cm, qJ, bw, fT, bXI, eM, nB, wK, vA, mx, U, vy, uJ, nw, dW, m, lS, du, hh, jM, ym, OD, iu, FW, dD, vl, lT, wh, aw, ht, NT, n, BH, aj, afE, fr, qN, JW, cd, ai, acy, TB, td, ke, jz, Wy, hw, aoc, oN, Nn, cU, lG, jy, dL, ey, h, aAG, uV, bl, Xh, eV, cue, pi, bXD, wC, hi, A, fJ, iw, ge, fQ, aq, fe, kp, zZ, cM, op, lx, JP, jq, bJ, kT, JR, GG, ar, Qy, bP, cO, dB, Fi, mg, hj, hL, E, uz, hR, at, bm, aMI, B, AN, L, bL, Dw, jv, fM, dA, AS, eR, fG, oe, eX, aA, cR, GP, cz, v, j, C, nS, V, GS, q, J, eA, Kx, GL, mP, yw, uT, lg, nX, aNA, MP, HC, aT, cy, Fx, acH, yb, bq, an, j, ae, af, eu, bA, jf, VC, kl, wr, bf, s, BA, iU, oV, Y, ev, r, iy, en, hf, bSy, t, cl, pb, ck, rR, nM, kP, ua, c, ac, hc, au, dh, gX, hr, dT, JL, mf, cS, dy, fD, bt, xb, ao, kV, vq, sY, kh, bPo, Ke, akd, aZ, aD, df, iW, jh, dG, eq, jE, OH, gb, tK, aue, ad, kL, hX, dP, fU, av, l, LL, hd, ib, Db, oz, py, Ot, ds, NS, lC, oj, QJ, hD, vc, vB, ji, lQ, jo, nJ, sj, uQ, uX, d, nU, cH, PE, ff, eH, zR, cr, Mz, cud, dq, gE, xv, aF, eZ, iV, Z, iR, KM, bz, qa, eK, bF, es, D, NC, mi, yE, bE, ag, z, eB, aam, gZ, aMy, aS, Kj, cP, ei, Zw, kS, X, y, acG); #c1(PPARG) c2(XP_011532146) c3(peroxisome proliferator-activated receptor gamma isoform X4) c4( ) c5(328175, 328176, 328177, 328178, 328179, 328180, 328181, 328182, 328183, 328184, 328185, 328186, 328187, 328188, 328189, 328190, 328191, 328192, 328193, 328194, 328195, 328196, 328197, 328198, 328199, 328200, 328201, 328202, 328203, 328204, 328205) c6( ) c7(ID, gh, T, bM, Ap, aC, fR, pf, YU, ea, if, e, al, fd, bB, ci, aTS, aon, mF, aR, ku, wD, dY, u, aE, iI, Fo, M, dV, cpD, K, qM, hu, dQ, H, eL, bc, eN, hm, aa, ly, yo, JI, aW, az, hl, LZ, nY, fc, vs, aB, g, hJ, ve, uC, eE, Vm, GR, eF, cm, qJ, bw, fT, bXI, eM, nB, wK, vA, mx, U, vy, uJ, nw, dW, m, lS, du, hh, jM, ym, OD, iu, FW, dD, vl, lT, wh, aw, ht, NT, n, BH, aj, afE, fr, qN, JW, cd, ai, acy, TB, td, ke, jz, Wy, hw, aoc, oN, Nn, CU, lG, jy, dL, ey, h, aAG, uV, bl, Xh, eV, cue, pi, bXD, wC, hi, A, fJ, iw, ge, fQ, aq, fe, kp, zZ, cM, op, lx, JP, jq, bJ, kT, JR, GG, ar, Qy, bP, c, dB, Fi, mg, hj, hL, E, uz, hR, at, bm, aMI, B, AN, L, bL, Dw, jv, fM, dA, AS, eR, fG, oe, eX, aA, cR, GP, cz, v, j, C, nS, V, GS, q, J, eA, Kx, GL, mP, yw, uT, lg, nX, aNA, MP, HC, aT, cy, Fx, acH, yb, bq, an, j, ae, af, eu, bA, jf, VC, kl, wr, bf, s, BA, iU, oV, Y, ev, r, iy, en, hf, bSy, t, cl, pb, ck, rR, nM, kP, ua, c, ac, hc, au, dh, gX, hr, dT, JL, mf, cS, dy, fD, bt, xb, ao, kV, vq, sY, kh, bPo, Ke, akd, aZ, aD, df, iW, jh, dG, eq, jE, OH, gb, tK, aue, ad, KL, hX, dP, fU, av, l, LL, hd, ib, Db, oz, py, Ot, ds, NS, lC, oj, QJ, HD, vc, vB, ji, lQ, jo, nJ, sj, uQ, uX, d, nU, cH, PE, ff, eH, zR, cr, Mz, cud, dq, gE, xv, aF, eZ, iV, Z, iR, KM, bz, qa, ek, bF, es, D, NC, mi, yE, bE, ag, z, eB, aam, gZ, aMy, aS, Kj, cP, ei, Zw, kS, X, y, acG); #c1(PPARGC1A) c2(NP_037393) c3(peroxisome proliferator-activated receptor gamma coactivator 1-alpha) c4(175227) c5(175228, 175229, 175230, 175231, 175232, 175233) c6( ) c7(ID, T, ea, e, bB, ci, aR, u, K, qM, brQ, bc, hm, ly, aW, fc, g, wT, vy, U, Hb, m, dD, vX, My, aw, aj, ct, cd, td, cc, eV, bl, pi, cgO, bux, bgn, aq, lx, bJ, W, cO, mg, ut, B, bL, eR, oe, jx, ex, cz, v, j, C, bX, V, J, jF, cF, yw, aT, bq, an, ae, af, kl, Y, r, kC, rY, OC, rR, ac, c, fP, mf, cS, gT, aen, aZ, bS, dG, fu, ah, eq, ad, BY, dP, hX, l, oW, ji, ul, uX, lR, Mz, dq, Z, es, nb, bE, gZ, ei, ol, kS); #c1(PPARGC1B) c2(NP_001166169) c3(peroxisome proliferator-activated receptor gamma coactivator 1-beta isoform 2) c4(68399) c5(68400, 68401, 68402, 68403, 68404, 68405) c6( ) c7(bL, bM, T, cz, bB, aj, dG, v, C, cd, J, ad, jF, cF, l, cc, u, aT, bl, K, dQ, brQ, ae, af, mR, aW, Y, g, bE, ut, ei); #c1(PPARGC1B) c2(NP_001166170) c3(peroxisome proliferator-activated receptor gamma coactivator 1-beta isoform 3) c4(68406) c5(68407, 68408, 68409, 68410, 68411, 68412, 68413) c6( ) c7(bL, bM, T, cz, bB, aj, dG, v, C, cd, J, ad, jF, cF, l, cc, u, aT, bl, K, dQ, brQ, ae, af, mR, aW, Y, g, bE, ut, ei); #c1(PPARGC1B) c2(NP_573570) c3(peroxisome proliferator-activated receptor gamma coactivator 1-beta isoform 1) c4(234558, 234559) c5(234560, 234561, 234562, 234563, 234564, 234565) c6( ) c7(bL, bM, T, cz, bB, aj, dG, v, C, cd, J, ad, jF, cF, l, cc, u, aT, bl, K, dQ, brQ, ae, af, mR, aW, Y, g, bE, ut, ei); #c1(PPARGC1B) c2(XP_011535855) c3(peroxisome proliferator-activated receptor gamma coactivator 1-beta isoform X1) c4( ) c5(333137, 333138, 333139, 333140, 333141, 333142) c6( ) c7(bL, bM, T, cz, bB, aj, dG, v, C, cd, J, ad, jF, cF, l, cc, u, aT, bl, K, dQ, brQ, ae, af, mR, aW, Y, g, bE, ut, ei); #c1(PPARGC1B) c2(XP_011535858) c3(peroxisome proliferator-activated receptor gamma coactivator 1-beta isoform X5) c4( ) c5(333143) c6( ) c7(bL, bM, T, cz, bB, aj, dG, v, C, cd, J, ad, jF, cF, l, cc, u, aT, bl, K, dQ, brQ, ae, af, mR, aW, Y, g, bE, ut, ei); #c1(PPAT) c2(NP_002694) c3(amidophosphoribosyltransferase proprotein) c4(131166) c5(131167, 131168, 131169, 131170, 131171, 131172) c6( ) c7(es, B, j, jp, bq); #c1(PPBP) c2(NP_002695) c3(platelet basic protein preproprotein) c4(131173, 131174, 131175, 131176, 131177, 131178, 131179, 131180, 131181, 131182, 131183, 131184) c5(131185, 131186, 131187, 131188, 131189, 131190) c6( ) c7(ck, bM, Dw, py, iU, qb, aW, ds, e, if, aT, cz, Ds, mN, j, K, qN, fr, axp, er, aDF); #c1(PPCDC) c2(NP_001288034) c3(phosphopantothenoylcysteine decarboxylase isoform e) c4( ) c5(113438) c6( ) c7(bN); #c1(PPCDC) c2(NP_068595) c3 (phosphopantothenoylcysteine decarboxylase isoform a) c4(209761) c5(209762, 209763, 209764, 209765, 209766, 209767) c6( ) c7(bN); #c1(PPEF1) c2(NP_689412) c3(serine/threonine-protein phosphatase with EF-hands 1 isoform 3) c4(242665) c5(242666, 242667, 242668, 242669, 242670, 242671, 242672, 242673, 242674, 242675, 242676, 242677) c6( ) c7(alV, an); #c1(PPEF1) c2(XP_005274610) c3(serine/threonine-protein phosphatase with EF-hands 1 isoform X1) c4(285193, 285194, 285195, 285196) c5(285197, 285198, 285199, 285200, 285201, 285202, 285203, 285204, 285205, 285206, 285207, 285208, 285209, 285210, 285211, 285212, 285213, 285214) c6( ) c7(alV, an); #c1(PPEF2) c2(XP_011530341) c3(serine/threonine-protein phosphatase with EF-hands 2 isoform X1) c4(326190, 326191) c5(326192, 326193, 326194, 326195, 326196, 326197, 326198, 326199, 326200, 326201, 326202, 326203) c6( ) c7(pN); #c1(PPFIA1) c2(NP_003617) c3(liprin-alpha-isoform b) c4(139661) c5(139662, 139663, 139664, 139665, 139666, 139667) c6( ) c7(T, aW, ff, al, ci, en, MC, ik); #c1(PPFIA1) c2(NP_803172) c3 (liprin-alpha-1 isoform a) c4(252517) c5(252518, 252519, 252520, 252521, 252522, 252523, 252524, 252525, 252526, 252527, 252528, 252529, 252530, 252531, 252532, 252533, 252534, 252535) c6( ) c7(T, aW, ff, aT, ci, en, MC, iK); #c1(PPFIA1) c2(XP_011543616) c3(liprin-alpha-1 isoform X9) c4(342622, 342623) c5( ) c6( ) c7(T, aW, ff, aT, ci, en, MC, iK); #c1(PPFIA1) c2(XP_011543618) c3(liprin-alpha-1 isoform X11) c4(342624) c5(342625, 342626, 342627, 342628, 342629) c6( ) c7(T, aW, ff, aT, ci, en, MC, ik); #c1(PPFIA1) c2(XP_011543621) c3(liprin-alpha-1 isoform X14) c4( ) c5(342630) c6( ) c7(T, aW, ff, aT, ci, en, MC, iK); #c1(PPFIA2) c2(NP_001207402) c3(liprin-alpha-2 isoform b) c4(83573, 83574) c5(83575, 83576, 83577, 83578, 83579, 83580, 83581, 83582, 83583, 83584, 83585, 83586) c6( ) c7(bM, aj, ad, Be); #c1(PPFIA2) c2(NP_001207403) c3(liprin-alpha-2 isoform c) c4(83587, 83588) c5(83589, 83590, 83591, 83592, 83593, 83594, 83595, 83596, 83597, 83598, 83599, 83600) c6( ) c7(bM, aj, ad, Be); #c1(PPFIA2) c2(NP_001207404) c3(liprin-alpha-2 isoform d) c4(83601) c5(83602, 83603, 83604, 83605, 83606, 83607, 83608, 83609, 83610, 83611, 83612, 83613) c6( ) c7(bM, aj, ad, Be); #c1(PPFIA2) c2(NP_001207405) c3(liprin-alpha-2 isoform e) c4(83614, 83615) c5(83616, 83617, 83618, 83619, 83620, 83621) c6( ) c7(bM, aj, ad, Be); #c1(PPFIA2) c2(NP_001207406) c3(liprin-alpha-2 isoform f) c4(83622) c5(83623, 83624, 83625, 83626, 83627, 83628) c6( ) c7(bM, aj, ad, Be); #c1(PPFIA2) c2(NP_001207407) c3(liprin-alpha-2 isoform g) c4(83629) c5(83630, 83631, 83632, 83633, 83634, 83635) c6( ) c7(bM, aj, ad, Be); #c1(PPFIA2) c2(NP_001207408) c3(liprin-alpha-2 isoform h) c4(83636) c5(83637, 83638, 83639, 83640, 83641) c6( ) c7(bM, aj, ad, Be); #c1(PPFIA2) c2(NP_001207409) c3(liprin-alpha-2 isoform i) c4( ) c5(83642, 83643, 83644, 83645, 83646) c6( ) c7(bM, aj, ad, Be); #c1(PPFIA2) c2(NP_003616) c3(liprin-alpha-2 isoform a) c4(139654) c5(139655, 139656, 139657, 139658, 139659, 139660) c6( ) c7(bM, aj, ad, Be); #c1(PPFIA2) c2(XP_006719728) c3(liprin-alpha-2 isoform X13) c4(292912) c5(292913) c6( ) c7(bM, aj, ad, Be); #c1(PPFIA2) c2(XP_006719731) c3(liprin-alpha-2 isoform X16) c4( ) c5(292914) c6( ) c7(bM, aj, ad, Be); #c1(PPFIA4) c2(NP_001291260) c3(liprin-alpha-4 isoform 1) c4(115610, 115611) c5(115612, 115613, 115614, 115615, 115616, 115617, 115618, 115619, 115620, 115621, 115622, 115623) c6( ) c7(fr, cz); #c1(PPFIA4) c2(NP_001291261) c3(liprin-alpha-4 isoform 2) c4(115624) c5(115625) c6( ) c7(fr, cz); #c1(PPFIA4) c2(XP_006711651) c3(liprin-alpha-4 isoform X5) c4(286185, 286186) c5(286187, 286188, 286189, 286190, 286191, 286192, 286193, 286194, 286195, 286196, 286197, 286198) c6( ) c7(fr, cz); #c1(PPFIA4) c2(XP_006711652) c3(liprin-alpha-4 isoform X6) c4( ) c5(286199, 286200, 286201, 286202, 286203) c6( ) c7(fr, cz); #c1(PPFIA4) c2(XP_006711654) c3(liprin-alpha-4 isoform X9) c4( ) c5(286204, 286205, 286206, 286207) c6(286208) c7(fr, cz); #c1(PPFIBP1) c2(NP_001185844) c3(liprin-beta-1 isoform 3) c4(76580) c5( ) c6( ) c7(hl, akV, kh, Td, Pg, it, ci, aFJ, V, fq); #c1(PPFIBP1) c2(NP_001185845) c3(liprin-beta-1 isoform 4) c4(76581) c5(76582, 76583, 76584, 76585, 76586, 76587, 76588, 76589, 76590, 76591, 76592, 76593) c6( ) c7(hl, akV, kh, Td, Pg, it, ci, aFJ, V, fq); #c1(PPFIBP1) c2(NP_003613) c3(liprin-beta-1 isoform 1) c4(139639) c5(139640, 139641, 139642, 139643, 139644, 139645, 139646) c6( ) c7(hl, akV, kh, Td, Pg, it, ci, aFJ, V, fq); #c1(PPFIBP1) c2(NP_803193) c3(liprin-beta-1 isoform 2) c4(252573) c5(252574, 252575, 252576, 252577, 252578, 252579, 252580, 252581, 252582, 252583, 252584, 252585) c6( ) c7(hl, akV, kh, Td, Pg, it, ci, aFJ, V, fq); #c1(PPFIBP1) c2(XP_005253564) c3(liprin-beta-1 isoform X2) c4(272572) c5( ) c6( ) c7(hl, akV, kh, Td, Pg, it, ci, aFJ, V, fq); #c1(PPFIBP1) c2(XP_005253571) c3(liprin-beta-1 isoform X7) c4(272573) c5( ) c6( ) c7(hl, akV, kh, Td, Pg, it, ci, aFJ, V, fq); #c1(PPFIBP1) c2(XP_006719224) c3(liprin-beta-1 isoform X11) c4(292336) c5(292337, 292338, 292339, 292340, 292341, 292342, 292343, 292344, 292345, 292346, 292347, 292348, 292349, 292350, 292351, 292352, 292353, 292354) c6( ) c7(hl, akV, kh, Td, Pg, it, ci, aFJ, V, fq); #c1(PPFIBP1) c2(XP_011519171) c3(liprin-beta-1 isoform X1) c4(313770) c5(313771, 313772, 313773, 313774, 313775) c6( ) c7(hl, akV, kh, Td, Pg, it, ci, aFJ, V, fq); #c1(PPFIBP2) c2(NP_001243497) c3(liprin-beta-2 isoform 2) c4( ) c5(90949, 90950, 90951, 90952, 90953, 90954, 90955, 90956, 90957, 90958) c6( ) c7(jz, aT); #c1(PPFIBP2) c2(NP_001243498) c3(liprin-beta-2 isoform 3) c4(90959) c5(90960, 90961, 90962, 90963, 90964, 90965, 90966, 90967, 90968, 90969, 90970, 90971) c6( ) c7(jz, aT); #c1(PPFIBP2) c2(NP_003612) c3(liprin-beta-2 isoform 1) c4(139606, 139607, 139608, 139609) c5(139610, 139611, 139612, 139613, 139614, 139615, 139616, 139617, 139618, 139619, 139620, 139621, 139622, 139623, 139624, 139625, 139626, 139627, 139628, 139629, 139630, 139631, 139632, 139633, 139634, 139635, 139636, 139637, 139638) c6( ) c7(jz, aT); #c1(PPFIBP2) c2(XP_006718413) c3(liprin-beta-2 isoform X5) c4(291831, 291832, 291833) c5( ) c6( ) c7(jz, aT); #c1(PPFIBP2) c2(XP_006718415) c3(liprin-beta-2 isoform X8) c4(291834) c5(291835, 291836, 291837, 291838, 291839, 291840, 291841) c6( ) c7(jz, aT); #c1(PPFIBP2) c2(XP_011518718) c3(liprin-beta-2 isoform X4) c4(313128) c5( ) c6( ) c7(jz, aT); #c1(PPIA) c2(NP_001287910) c3(peptidyl-prolyl cis-trans isomerase A isoform 2) c4( ) c5(113179, 113180, 113181, 113182, 113183) c6( ) c7(bL, bM, T, dD, fR, ea, lH, ha, jx, aA, aj, eq, bX, eJ, J, ad, av, ft, jz, aE, ds, aT, h, K, ae, afu, d, bf, aW, ff, wc, lo, kp, en, cl, pl, bz, ck, iQ, c, eM, hL, bm, aS, lC, B, y); #c1(PPIA) c2(NP_066953) c3(peptidyl-prolyl cis-trans isomerase A isoform 1) c4(207685, 207686, 207687) c5(207688, 207689, 207690, 207691, 207692, 207693, 207694, 207695, 207696, 207697, 207698, 207699, 207700) c6( ) c7(bL, bM, T, dD, fR, ea, lH, ha, jx, aA, aj, eq, bX, eJ, J, ad, av, ft, jz, aE, ds, aT, h, K, ae, afu, d, bf, aW, ff, wc, lo, kp, en, cl, pl, bz, ck, iQ, c, eM, hL, bm, aS, lC, B, y); #c1(PPIB) c2(NP_000933) c3(peptidyl-prolyl cis-trans isomerase B precursor) c4(17506) c5(17507, 17508, 17509, 17510, 17511, 17512) c6( ) c7(ck, T, bE, aW, jx, aT, bB, aq, h, K, zp, cuf, B); #c1(PPIC) c2(NP_000934) c3(peptidyl-prolyl cis-trans isomerase C precursor) c4(17513) c5(17514, 17515, 17516, 17517, 17518, 17519) c6( ) c7(aF, aA); #c1(PPID) c2(NP_005029) c3(peptidyl-prolyl cis-trans isomerase D) c4(152297) c5(152298, 152299, 152300, 152301, 152302, 152303) c6( ) c7(T, aE, dH, Ae, aW, dF, e, xs, U, aT, jh, K, ae, af, ad); #c1(PPIF) c2(NP_005720) c3(peptidyl-prolyl cis-trans isomerase F, mitochondrial precursor) c4(158813, 158814) c5(158815, 158816, 158817, 158818, 158819, 158820, 158821) c6( ) c7(T, anR, aE, dH, Ae, aW, e, ju, xs, oj, aT, U, K, dF, af); #c1(PPIF) c2(XP_005269436) c3(peptidyl-prolyl cis-trans isomerase F, mitochondrial isoform X1) c4(282565) c5(282566, 282567, 282568, 282569, 282570, 282571, 282572, 282573, 282574, 282575, 282576) c6( ) c7(T, anR, aE, dH, Ae, aW, e, ju, xs, oj, aT, U, K, dF, af); #c1(PPIG) c2(NP_004783) c3(peptidyl-prolyl cis-trans isomerase G) c4(149593, 149594, 149595, 149596) c5(149597, 149598, 149599, 149600, 149601, 149602, 149603, 149604, 149605, 149606, 149607, 149608, 149609, 149610, 149611, 149612, 149613, 149614) c6( ) c7(bM, T, ea, e, al, fd, bB, lR, ci, bh, XW, bW, dV, iv, K, hu, eL, aAu, aW, hl, mK, eM, vA, bcl, dD, ht, ct, qN, cd, h, eV, aNU, dL, hB, he, Nu, sp, auM, aq, op, B, qc, bL, ex, aA, CR, cz, j, C, q, eA, aT, cy, rE, ae, af, iU, Lf, r, lo, HL, en, bu, pb, ck, ac, c, dh, gT, bt, ha, bS, eq, jE, hF, ad, dP, av, HF, io, lU, ff, eZ, aF, Z, bz, ek, gk, ml, bE); #c1(PPIG) c2(XP_011510494) c3(peptidyl-prolyl cis-trans isomerase G isoform X2) c4( ) c5(301006, 301007, 301008, 301009, 301010, 301011, 301012, 301013, 301014, 301015) c6( ) c7(bM, T, ea, e, al, fd, bB, lR, ci, bh, XW, bW, dV, iv, K, hu, eL, aAu, aW, hl, mK, eM, vA, bcl, dD, ht, ct, qN, cd, h, eV, aNU, dL, hB, he, Nu, sp, auM, aq, op, B, qc, bL, eX, aA, CR, cz, j, C, q, eA, aT, cy, rE, ae, af, iU, Lf, r, lo, HL, en, bu, pb, ck, ac, c, dh, gT, bt, ha, bS, eq, jE, hF, ad, dP, av, HF, io, lU, ff, eZ, aF, Z, bz, ek, gk, mO, bE); #c1(PPIL1) c2(NP_057143) c3(peptidyl-prolyl cis-trans isomerase-like 1) c4(189096) c5(189097, 189098, 189099, 189100, 189101, 189102) c6( ) c7(T, bE, jx, aA, bB, aF); #c1(PPIL2) c2(NP_680480) c3(peptidyl-prolyl cis-trans isomerase-like 2 isoform a) c4(242306) c5(242307, 242308, 242309, 242310, 242311, 242312, 242313, 242314, 242315, 242316, 242317, 242318, 242319, 242320, 242321, 242322, 242323, 242324) c6( ) c7(af); #c1(PPIL2) c2(NP_680481) c3(peptidyl-prolyl cis-trans isomerase-like 2 isoform b) c4(242325) c5(242326, 242327, 242328, 242329, 242330, 242331) c6( ) c7(af); #c1(PPIL2) c2(XP_011528345) c3(peptidyl-prolyl cis-trans isomerase-like 2 isoform X4) c4(323389) c5(323390) c6( ) c7(af); #c1(PPIL2) c2(XP_011528346) c3(peptidyl-prolyl cis-trans isomerase-like 2 isoform X5) c4(323391) c5(323392, 323393, 323394, 323395, 323396) c6( ) c7(af); #c1(PPIL2) c2(XP_011528353) c3(peptidyl-prolyl cis-trans isomerase-like 2 isoform X7) c4( ) c5(323397) c6( ) c7(af); #c1(PPIL3) c2(NP_115861) c3(peptidyl-prolyl cis-trans isomerase-like 3 isoform PPIL3a) c4(224372) c5(224373, 224374, 224375, 224376, 224377, 224378, 224379, 224380, 224381, 224382) c6( ) c7(eA); #c1(PPIL3) c2(NP_570981) c3(peptidyl-prolyl cis-trans isomerase-like 3 isoform PPIL3b) c4(234453, 234454) c5(234455, 234456, 234457, 234458, 234459, 234460, 234461, 234462, 234463, 234464, 234465, 234466) c6( ) c7(eA); #c1(PPIPSK1) c2(NP_001124330) c3(inositol hexakisphosphate and diphosphoinositol-pentakisphosphate kinase 1 isoform 5) c4(48689) c5(48690, 48691, 48692, 48693, 48694, 48695, 48696, 48697, 48698, 48699, 48700, 48701, 48702, 48703) c6( ) c7(Y, jx, cU, aiT, lo, pm); #c1(PPIPSK1) c2(NP_001177 (43) c3 (inositol hexakisphosphate and diphosphoinositol-pentakisphosphate kinase 1 isoform 6) c4(72465, 72466, 72467, 72468) c5(72469, 72470, 72471, 72472, 72473, 72474, 72475, 72476, 72477, 72478, 72479, 72480, 72481, 72482, 72483, 72484, 72485, 72486, 72487, 72488, 72489, 72490, 72491) c6( ) c7(Y, jx, cU, aiT, lo, pm); #c1(PPIPSK1) c2(XP_005254861) c3(inositol hexakisphosphate and diphosphoinositol-pentakisphosphate kinase 1 isoform X1) c4(273354) c5(273355, 273356, 273357, 273358, 273359, 273360, 273361, 273362, 273363, 273364, 273365, 273366, 273367, 273368) c6( ) c7(Y, jx, cU, aiT, lo, pm); #c1(PPIPSK1) c2(XP_006720849) c3(inositol hexakisphosphate and diphosphoinositol-pentakisphosphate kinase 1 isoform X7) c4(293725) c5(293726, 293727, 293728, 293729, 293730, 293731, 293732, 293733, 293734, 293735, 293736, 293737, 293738, 293739, 293740) c6( ) c7(Y, jx, cl, aiT, lo, pm); #c1(PPIPSK1) c2(XP_011520556) c3(inositol hexakisphosphate and diphosphoinositol-pentakisphosphate kinase 1 isoform X2) c4(315476) c5(315477, 315478, 315479, 315480, 315481, 315482, 315483, 315484, 315485, 315486, 315487, 315488, 315489) c6( ) c7(Y, jx, cu, ail, lo, pm); #c1(PPIPSK1) c2(XP_011520557) c3(inositol hexakisphosphate and diphosphoinositol-pentakisphosphate kinase 1 isoform X3) c4(315490) c5( ) c6( ) c7(Y, jx, cl, aiT, lo, pm); #c1(PPIPSK1) c2(XP_011520560) c3(inositol hexakisphosphate and diphosphoinositol-pentakisphosphate kinase 1 isoform X8) c4( ) c5(315491) c6( ) c7(Y, jx, cU, ail, lo, pm); #c1(PPL) c2(NP_002696) c3(periplakin) c4(131191) c5(131192, 131193, 131194, 131195, 131196, 131197) c6( ) c7(ay, T, hd, ff, ht, yb, hu, en, rz, xt); #c1(PPM1A) c2(NP_066283) c3(protein phosphatase (A isoform 1) c4( ) c5(206974, 206975, 206976, 206977, 206978, 206979, 206980, 206981) c6( ) c7(T, h, lo, ae, B, ad); #c1(PPM1A) c2(NP_808820) c3(protein phosphatase (A isoform 2) c4(252951) c5(252952, 252953, 252954, 252955, 252956, 252957, 252958, 252959, 252960, 252961, 252962, 252963, 252964, 252965, 252966, 252967, 252968, 252969, 252970, 252971, 252972) c6( ) c7(T, h, lo, ae, B, ad); #c1(PPM1A) c2(NP_808821) c3(protein phosphatase IA isoform 3) c4(252973, 252974, 252975, 252976, 252977) c5(252978, 252979, 252980, 252981, 252982, 252983, 252984, 252985, 252986, 252987) c6( ) c7(T, h, lo, ae, B, ad); #c1(PPM1A) c2(XP_011535179) c3(protein phosphatase 1A isoform X3) c4( ) c5(332022) c6( ) c7(T, h, lo, ae, B, ad); #c1(PPM1B) c2(NP_001028729) c3(protein phosphatase (B isoform 5) c4( ) c5(30535, 30536, 30537, 30538, 30539, 30540, 3054I, 30542, 30543, 30544, 30545, 30546, 30547, 30548, 30549, 30550, 30551, 30552) c6( ) c7(azB, azC, j); #c1(PPM1B) c2(NP_002697) c3(protein phosphatase IB isoform 1) c4(131198, 131199, 131200, 131201) c5(131202, 131203, 131204, 131205, 131206, 131207, 131208) c6( ) c7(azB, azC, j); #c1(PPM1B) c2(NP_808907) c3(protein phosphatase IB isoform 2) c4(253009) c5(253010, 253011, 253012, 253013, 253014, 253015) c6( ) c7(azB, azC, j); #c1(PPM1B) c2(NP_808908) c3(protein phosphatase 1B isoform 3) c4(253016) c5(253017, 253018, 253019, 253020, 253021) c6( ) c7(azB, azC, j); #c1(PPM1D) c2(NP_003611) c3(protein phosphatase ID) c4(139585, 139586, 139587) c5(139588, 139589, 139590, 139591, 139592, 139593, 139594, 139595, 139596, 139597, 139598, 139599, 139600, 139601, 139602, 139603, 139604, 139605) c6( ) c7(T, yk, aA, ci, lu, q, eA, aT, K, jf, B, aW, oV, r, eS, as, cm, t, eM, X, cX, m, cug); #c1(PPM1E) c2(NP_055721) c3(protein phosphatase (E) c4(183058, 183059) c5(183060, 183061, 183062, 183063, 183064, 183065, 183066, 183067, 183068, 183069, 183070, 183071) c6( ) c7(aW, aT); #c1(PPM1F) c2(NP_055449) c3(protein phosphatase IF) c4(181220, 181221, 181222, 181223) c5(181224, 181225, 181226, 181227, 181228, 181229, 181230, 181231, 181232, 181233, 181234, 181235, 181236, 181237, 181238, 181239, 181240, 181241, 181242, 181243) c6( ) c7(T, K); #c1(PPM1F) c2(XP_011528858) c3(protein phosphatase IF isoform X1) c4( ) c5(324241, 324242, 324243, 324244, 324245, 324246, 324247, 324248) c6( ) c7(T, K); #c1(PPM1G) c2(NP_817092) c3(protein phosphatase 1G) c4(253089, 253090, 253091) c5(253092, 253093, 253094, 253095, 253096, 253097, 253098, 253099, 253100, 253101, 253102, 253103, 253104, 253105, 253106, 253107, 253108, 253109, 253110, 253111, 253112, 253113) c6( ) c7(cz); #c1(PPM1H) c2(NP_065751) c3(protein phosphatase 1H) c4(205183) c5(205184, 205185, 205186, 205187, 205188, 205189) c6( ) c7(bE, ev, eZ, bB, g, fq); #c1(PPM1K) c2(NP_689755) c3(protein phosphatase IK, mitochondrial) c4(243873, 243874) c5(243875, 243876, 243877, 243878, 243879, 243880) c6( ) c7(cuh, ac, c, bP, agm, K, Gp, B); #c1(PPM1K) c2(XP_005262832) c3(protein phosphatase IK, mitochondrial isoform X3) c4( ) c5(278125, 278126, 278127, 278128, 278129, 278130, 278131, 278132, 278133, 278134) c6( ) c7(cuh, ac, c, bP, agm, K, Gp, B); #c1(PPM1L) c2(NP_640338) c3(protein phosphatase 1L) c4(238458) c5(238459, 238460, 238461, 238462, 238463, 238464) c6( ) c7(bF, lf, v); #c1(PPM1L) c2(XP_006713570) c3(protein phosphatase IL isoform X1) c4(287231) c5(287232, 287233, 287234, 287235, 287236, 287237) c6( ) c7(bF, lf, v); #c1(PPM1M) c2(NP_001116342) c3(protein phosphatase IM isoform b) c4( ) c5(43792, 43793, 43794, 43795, 43796) c6( ) c7(aW, Ld, bK, aT); #c1(PPM1M) c2(NP_653242) c3(protein phosphatase IM isoform a) c4(239280) c5(239281, 239282, 239283, 239284, 239285, 239286, 239287, 239288) c6( ) c7(aW, Ld, bk, aT); #c1(PPM1M) c2(XP_005264937) c3(protein phosphatase IM isoform X2) c4( ) c5(279310) c6( ) c7(aW, Ld, bK, aT); #c1(PPM1M) c2(XP_005264938) c3(protein phosphatase IM isoform X3) c4(279311, 279312, 279313) c5(279314, 279315, 279316, 279317, 279318, 279319, 279320, 279321, 279322, 279323) c6( ) c7(aW, Ld, bK, aT); #c1(PPME1) c2(NP_001258522) c3(protein phosphatase methylesterase 1 isoform b) c4(96096) c5(96097, 96098, 96099, 96100, 96101, 96102) c6( ) c7(T, AM, hL, aA, aF, as, cl, bz, eA); #c1(PPMEI) c2(NP_057231) c3(protein phosphatase methylesterase 1 isoform a) c4(189613, 189614) c5(189615, 189616, 189617, 189618, 189619, 189620, 189621, 189622, 189623, 189624, 189625, 189626) c6( ) c7(T, AM, hL, aA, aF, as, cl, bz, eA); #c1(PPOX) c2(NP_001116236) c3(protoporphyrinogen oxidase) c4(43699) c5( ) c6( ) c7(bL, cuj, cui, Ow, aVf, QW, gi, nl, nh, QV, dq, bHQ, bKa, GR, OC); #c1(PPOX) c2(XP_011507967) c3(protoporphyrinogen oxidase isoform X3) c4(298399) c5(298400, 298401, 298402, 298403, 298404, 298405) c6( ) c7(bL, cuj, cui, Ow, aVf, QW, gi, nl, nh, QV, dq, bHQ, bKa, GR, OC); #c1(PPOX) c2(XP_011507973) c3(protoporphyrinogen oxidase isoform X9) c4( ) c5(298406, 298407, 298408, 298409, 298410, 298411, 298412, 298413, 298414, 298415) c6( ) c7(bL, cuj, cui, Ow, aVf, QW, gi, nl, nh, QV, dq, bHQ, bKa, GR, OC); #c1(PPOX) c2(XP_011507974) c3(protoporphyrinogen oxidase isoform X10) c4( ) c5(298416) c6( ) c7(bL, cuj, cui, Ow, aVf, QW, gi, nl, nh, QV, dq, bHQ, bKa, GR, OC); #c1(PPOX) c2(XP_011507977) c3 (protoporphyrinogen oxidase isoform X13) c4(298417) c5(298418, 298419, 298420, 298421, 298422) c6( ) c7(bL, cuj, cui, Ow, aVf, QW, gi, nl, nh, QV, dq, bHQ, bKa, GR, OC); #c1(PPP1CA) c2(NP_001008709) c3(serine/threonine-protein phosphatase PPI-alpha catalytic subunit isoform 3) c4(23381) c5(23382, 23383, 23384, 23385, 23386, 23387) c6( ) c7(bxC, T, d, aW, ff, wc, al, en, B); #c1(PPP1CA) c2(NP_002699) c3(serine/threonine-protein phosphatase PPI-alpha catalytic subunit isoform 1) c4(131209, 131210) c5(131211, 131212, 131213, 131214, 131215, 131216, 131217, 131218, 131219, 131220, 131221, 131222) c6( ) c7(bxC, T, d, aW, ff, wc, aT, en, B); #c1(PPP1CA) c2(NP_996756) c3(serine/threonine-protein phosphatase PPI-alpha catalytic subunit isoform 2) c4(264480) c5(264481, 264482, 264483, 264484, 264485, 264486) c6( ) c7(bxC, T, d, aW, ff, wc, al, en, B); #c1(PPP1CC) c2(NP_001231903) c3(serine/threonine-protein phosphatase PPI-gamma catalytic subunit isoform 2) c4(87687) c5(87688, 87689, 87690, 87691, 87692, 87693) c6( ) c7(vH, t, T, gU, ND); #c1(PPP1CC) c2(NP_002701) c3(serine/threonine-protein phosphatase PPI-gamma catalytic subunit isoform 1) c4(131223, 131224, 131225) c5(131226, 131227, 131228, 131229, 131230, 131231, 131232, 131233, 131234, 131235, 131236, 131237, 131238, 131239, 131240, 131241, 131242, 131243) c6( ) c7(vH, t, T, gU, ND); #c1(PPP1R10) c2(NP_002705) c3(serine/threonine-protein phosphatase 1 regulatory subunit 10) c4(131262, 131263) c5(131264, 131265, 131266, 131267, 131268, 131269, 131270, 131271, 131272, 131273, 131274, 131275, 131276, 131277, 131278, 131279, 131280, 131281, 131282, 131283, 131284, 131285, 131286, 131287, 131288, 131289, 131290, 131291, 131292, 131293, 131294, 131295, 131296, 131297, 131298, 131299, 131300, 131301, 131302, 131303, 131304, 131305, 131306, 131307, 131308, 131309, 131310, 131311, 131312, 131313) c6( ) c7(T, afF, eZ, io, as, hB, hH, af); #c1(PPP1R11) c2(NP_068778) c3(protein phosphatase 1 regulatory subunit 11) c4(210469, 210470) c5(210471, 210472, 210473, 210474, 210475, 210476, 210477, 210478, 210479, 210480, 210481, 210482, 210483, 210484, 210485, 210486, 210487, 210488, 210489, 210490, 210491, 210492, 210493, 210494, 210495, 210496, 210497, 210498, 210499, 210500, 210501) c6( ) c7(T, aE, W, xl, eZ, nY, cm, V); #c1(PPP1R11) c2(XP_006715237) c3(protein phosphatase 1 regulatory subunit Il isoform X1) c4( ) c5(288868, 288869, 288870, 288871, 288872, 288873, 288874, 288875, 288876, 288877, 288878, 288879, 288880, 288881, 288882, 288883, 288884, 288885, 288886, 288887, 288888, 288889, 288890, 288891, 288892, 288893, 288894, 288895, 288896, 288897, 288898, 288899, 288900, 288901, 288902, 288903, 288904, 288905, 288906, 288907, 288908, 288909, 288910, 288911, 288912, 288913, 288914, 288915, 288916, 288917, 288918, 288919, 288920, 288921, 288922, 288923, 288924, 288925, 288926, 288927, 288928, 288929, 288930, 288931, 288932, 288933, 288934, 288935, 288936, 288937, 288938, 288939, 288940, 288941, 288942, 288943, 288944, 288945, 288946, 288947, 288948, 288949, 288950, 288951, 288952, 288953, 288954, 288955, 288956, 288957, 288958, 288959, 288960, 288961, 288962, 288963, 288964, 288965, 288966, 288967, 288968, 288969, 288970, 288971, 288972, 288973, 288974, 288975, 288976, 288977, 288978, 288979, 288980, 288981, 288982, 288983, 288984, 288985, 288986, 288987, 288988, 288989, 288990, 288991, 288992, 288993, 288994, 288995, 288996, 288997, 288998, 288999, 289000, 289001, 289002, 289003, 289004, 289005, 289006, 289007, 289008, 289009, 289010) c6( ) c7(T, aE, W, xl, eZ, nY, cm, V); #c1(PPP1R12A) c2(NP_001137357) c3 (protein phosphatase 1 regulatory subunit 12A isoform a) c4(55581, 55582) c5(55583, 55584, 55585, 55586, 55587, 55588, 55589, 55590, 55591, 55592, 55593, 55594, 55595, 55596, 55597, 55598, 55599) c6( ) c7(bM, T, ib, bf, aE, hW, fd, akA, afV, m, cd); #c1(PPP1R12A) c2(NP_001137358) c3 (protein phosphatase 1 regulatory subunit (ZA isoform b) c4(55600) c5(55601, 55602, 55603, 55604, 55605) c6( ) c7(bM, T, ib, bf, aE, hW, fd, akA, afV, m, cd); #c1(PPP1R12A) c2(NP_001231919) c3(protein phosphatase 1 regulatory subunit 12A isoform c) c4(87701, 87702) c5(87703, 87704, 87705, 87706, 87707, 87708, 87709, 87710, 87711, 87712, 87713, 87714) c6( ) c7(bM, T, ib, bf, aE, hW, fd, akA, afV, m, cd); #c1(PPP1R12A) c2(NP_001231921) c3(protein phosphatase 1 regulatory subunit 12A isoform d) c4(87715, 87716) c5(87717, 87718, 87719, 87720, 87721, 87722, 87723, 87724, 87725, 87726, 87727, 87728) c6( ) c7(bM, T, ib, bf, aE, hW, fd, akA, afV, m, cd); #c1(PPP1R12A) c2(XP_011536685) c3(protein phosphatase 1 regulatory subunit 12A isoform X11) c4(334189) c5(334190, 334191, 334192, 334193, 334194, 334195) c6( ) c7(bM, T, ib, bf, aE, hW, fd, akA, afV, m, cd); #c1(PPP1R12B) c2(NP_001161329) c3(protein phosphatase 1 regulatory subunit (28 isoform e) c4(65610) c5(65611, 65612, 65613, 65614, 65615, 65616) c6( ) c7(ac); #c1(PPP1R12B) c2(NP_001161330) c3 (protein phosphatase 1 regulatory subunit (28 isoform f) c4(65617) c5(65618, 65619, 65620, 65621, 65622, 65623) c6( ) c7(ac); #c1(PPP1R12B) c2(NP_002472) c3(protein phosphatase 1 regulatory subunit 128 isoform a) c4(129126, 129127, 129128) c5(129129, 129130, 129131, 129132, 129133, 129134, 129135, 129136, 129137, 129138, 129139, 129140, 129141, 129142) c6( ) c7(ac); #c1(PPP1R(2B) c2(NP_115286) c3(protein phosphatase 1 regulatory subunit 12B isoform c) c4( ) c5(222830, 222831, 222832, 222833, 222834) c6( ) c7(ac); #c1(PPP1R(2B) c2(NP_115287) c3(protein phosphatase 1 regulatory subunit 128 isoform d) c4(222835) c5(222836, 222837, 222838, 222839, 222840) c6( ) c7(ac); #c1(PPP1R12C) c2(NP_060077) c3(protein phosphatase 1 regulatory subunit 12C isoform a) c4(194042, 194043) c5(194044, 194045, 194046, 194047, 194048, 194049, 194050, 194051, 194052, 194053, 194054, 194055) c6( ) c7(T, oT, cA, eM, bE, aW, aZ, aT, fd, aq, eA, ei, B); #c1(PPP1R12C) c2(XP_005259070) c3(protein phosphatase 1 regulatory subunit (2C isoform X1) c4(275948) c5(275949, 275950, 275951, 275952, 275953, 275954, 275955) c6( ) c7(T, oT, cA, eM, bE, aW, aZ, aT, fd, aq, eA, ei, B); #c1(PPP1R13B) c2(NP_056131) c3(apoptosis-stimulating of p53 protein 1) c4(185828, 185829, 185830, 185831) c5(185832, 185833, 185834, 185835, 185836, 185837, 185838, 185839, 185840, 185841, 185842, 185843, 185844, 185845, 185846, 185847, 185848, 185849) c6( ) c7(T, aZ, fd, eA, hX, x, oT, aoc, aT, nJ, ZH, CN, aW, Y, aq, cA, eM, bE, hk, k, ei, B); #c1(PPP1R13B) c2(XP_011534900) c3(apoptosis-stimulating of p53 protein 1 isoform X7) c4( ) c5(331422, 331423, 331424, 331425, 331426) c6( ) c7(T, aZ, fd, eA, hX, x, oT, aoc, aT, nJ, ZH, CN, aW, Y, aq, cA, eM, bE, hk, k, ei, B); #c1(PPP1R13L) c2(NP_001135974) c3(relA-associated inhibitor) c4(53879, 53880) c5(53881, 53882, 53883, 53884, 53885, 53886, 53887, 53888, 53889, 53890, 53891, 53892, 53893, 53894, 53895, 53896, 53897) c6( ) c7(T, NT, fd, n, ci, lu, hF, ad, zn, hX, x, hd, bnj, aT, dL, ae, d, s, aW, Y, Lf, r, bu, t, bz, ck, sS, eM, nB, cj, m, k, bt, B); #c1(PPP1R14A) c2(NP_001230876) c3(protein phosphatase 1 regulatory subunit 14A isoform 2) c4(86901) c5(86902, 86903, 86904, 86905, 86906, 86907) c6( ) c7(ib, blU, bzS, cuk, HR, B); #c1(PPP1R14A) c2(NP_150281) c3(protein phosphatase 1 regulatory subunit 14A isoform 1) c4(228028) c5(228029, 228030, 228031, 228032, 228033, 228034) c6( ) c7(ib, blU, bzS, cuk, HR, B); #c1(PPP1R14B) c2(NP_619634) c3(protein phosphatase 1 regulatory subunit 14B) c4(236899) c5(236900, 236901, 236902, 236903, 236904, 236905) c6( ) c7(h, jZ, Y, aaF); #c1(PPP1R14C) c2(NP_112211) c3 (protein phosphatase 1 regulatory subunit 14C) c4(220848) c5(220849, 220850, 220851, 220852, 220853, 220854) c6( ) c7(GC, aW, lf, aT); #c1(PPP1R15A) c2(NP_055145) c3(protein phosphatase 1 regulatory subunit 15A) c4(179414, 179415, 179416) c5(179417, 179418, 179419, 179420, 179421, 179422, 179423, 179424, 179425, 179426, 179427, 179428, 179429, 179430, 179431, 179432, 179433, 179434) c6( ) c7(eW, T, hl, eR, nO, Y, ns, rf, fd, cy, j, ci, K, eA); #c1(PPP1R15B) c2(NP_116222) c3(protein phosphatase 1 regulatory subunit 15B) c4(225837, 225838) c5(225839, 225840, 225841, 225842, 225843, 225844, 225845, 225846, 225847, 225848, 225849, 225850) c6( ) c7(aW, aT); #c1(PPP1R17) c2(NP_001138595) c3(protein phosphatase 1 regulatory subunit 17 isoform 2) c4(57148) c5(57149, 57150, 57151, 57152, 57153, 57154) c6( ) c7(cH, c1); #c1(PPP1R17) c2(NP_006649) c3(protein phosphatase I regulatory subunit 17 isoform 1) c4(166880) c5(166881, 166882, 166883, 166884, 166885, 166886) c6( ) c7(cH, c1); #c1(PPP1R18) c2(NP_001128342) c3(phostensin) c4(49862, 49863, 49864, 49865, 49866, 49867) c5(49868, 49869, 49870, 49871, 49872, 49873, 49874, 49875, 49876, 49877, 49878, 49879, 49880, 49881, 49882, 49883, 49884, 49885, 49886, 49887, 49888, 49889, 49890, 49891, 49892, 49893, 49894, 49895, 49896, 49897, 49898, 49899, 49900, 49901, 49902, 49903, 49904, 49905, 49906, 49907, 49908, 49909, 49910, 49911, 49912, 49913, 49914, 49915, 49916, 49917, 49918, 49919, 49920, 49921, 49922, 49923, 49924, 49925, 49926, 49927, 49928, 49929, 49930, 49931, 49932, 49933, 49934, 49935, 49936, 49937, 49938, 49939, 49940, 49941, 49942, 49943, 49944, 49945, 49946, 49947, 49948, 49949, 49950) c6( ) c7(eZ); #c1(PPP1R18) c2(XP_003403645) c3 (phostensin isoform X1) c4( ) c5(266115, 266116, 266117, 266118, 266119, 266120, 266121, 266122, 266123) c6( ) c7(eZ); #c1(PPP1R1A) c2(NP_006732) c3(protein phosphatase 1 regulatory subunit (A) c4(167540) c5(167541, 167542, 167543, 167544, 167545, 167546) c6( ) c7(T, aw, eX, fd, aks, C, ai, cd, cc, aT, eV, hm, d, xR, aW, Y, t, bF, iO, eL, iT, aKp, aS, m, B); #c1(PPP1R1A) c2(XP_006719534) c3(protein phosphatase 1 regulatory subunit (A isoform X2) c4(292632) c5(292633, 292634, 292635, 292636, 292637, 292638) c6( ) c7(T, aw, eX, fd, aks, C, ai, cd, cc, aT, eV, hm, d, xR, aW, Y, t, bF, iO, eL, iT, aKp, aS, m, B); #c1(PPP1R1B) c2(NP_115568) c3(protein phosphatase 1 regulatory subunit 1B isoform 1) c4(223215, 223216, 223217) c5(223218, 223219, 223220, 223221, 223222, 223223, 223224, 223225, 223226, 223227, 223228, 223229, 223230, 223231, 223232, 223233, 223234, 223235) c6( ) c7(tJ, T, aA, GC, bS, ct, xr, ci, AO, aT, bK, JI, aW, Gv, ff, aF, en, g, Z, jb, FT, ok, rR, cA, Kv, sv); #c1(PPP1R1B) c2(NP_852606) c3(protein phosphatase 1 regulatory subunit (B isoform 2) c4( ) c5(255047, 255048, 255049, 255050, 255051, 255052, 255053, 255054) c6( ) c7(tJ, T, aA, GC, bS, ct, xr, ci, AO, aT, bK, JI, aW, Gv, ff, aF, en, g, Z, jb, FT, ok, rR, cA, Kv, sv); #c1(PPP1R2) c2(NP_006232) c3(protein phosphatase inhibitor 2 isoform 2) c4(163137) c5(163138, 163139, 163140, 163141, 163142, 163143) c6( ) c7(bL, T, ey, K); #c1(PPP1R3A) c2(NP_002702) c3(protein phosphatase 1 regulatory subunit 3A) c4(131244) c5(131245, 131246, 131247, 131248, 131249, 131250) c6( ) c7(td, bL, T, kl, bKq, eR, Mm, iT, aZ, QJ, jh, r, aj, mf, j, ei, af, J, ad); #c1(PPP1R3B) c2(NP_078883) c3(protein phosphatase 1 regulatory subunit 3B) c4(215883) c5(215884, 215885, 215886, 215887, 215888, 215889, 215890, 215891, 215892, 215893) c6( ) c7(ag, fc, yT, aj, v, bK, j, cP, ei, dQ, J); #c1(PPP1R3C) c2(NP_005389) c3(protein phosphatase 1 regulatory subunit 3C) c4(155658, 155659, 155660) c5(155661, 155662, 155663, 155664, 155665, 155666, 155667, 155668, 155669, 155670, 155671, 155672, 155673, 155674, 155675, 155676, 155677, 155678) c6( ) c7(bL, ag, yT, aj, dQ, azp, cd, J); #c1(PPP1R42) c2(NP_001013648) c3(protein phosphatase 1 regulatory subunit 42) c4(25681, 25682, 25683) c5(25684, 25685, 25686, 25687, 25688, 25689, 25690, 25691, 25692, 25693, 25694, 25695, 25696, 25697, 25698, 25699, 25700, 25701) c6( ) c7(aw, g); #c1(PPP1R7) c2(NP_001269338) c3(protein phosphatase 1 regulatory subunit 7 isoform 2) c4(101162) c5(101163, 101164, 101165, 101166, 101167, 101168) c6( ) c7(T, ci, qN); #c1(PPP1R7) c2(NP_001269339) c3(protein phosphatase 1 regulatory subunit 7 isoform 3) c4(101169) c5(101170, 101171, 101172, 101173, 101174, 101175) c6( ) c7(T, ci, qN); #c1(PPP1R7) c2(NP_001269340) c3(protein phosphatase I regulatory subunit 7 isoform 4) c4(101176) c5(101177, 101178, 101179, 101180, 101181, 101182) c6( ) c7(T, ci, qN); #c1(PPP1R7) c2(NP_002703) c3(protein phosphatase 1 regulatory subunit 7 isoform 1) c4(131251) c5(131252, 131253, 131254, 131255, 131256, 131257, 131258, 131259, 131260, 131261) c6( ) c7(T, ci, qN); #c1(PPP1R9A) c2(NP_001159632) c3 (neurabin-1 isoform 1) c4(64492) c5(64493, 64494, 64495, 64496, 64497, 64498) c6( ) c7(y); #c1(PPP1R9A) c2(NP_001159633) c3(neurabin-1 isoform 2) c4(64499) c5(64500, 64501, 64502, 64503, 64504, 64505) c6( ) c7(y); #c1(PPP1R9A) c2(NP_001159634) c3(neurabin-1 isoform 3) c4(64506) c5(64507, 64508, 64509, 64510, 64511, 64512) c6( ) c7(y); #c1(PPP1R9A) c2(NP_001159635) c3(neurabin-1 isoform 5) c4(64513) c5(64514, 64515) c6( ) c7(y); #c1(PPP1R9A) c2(NP_060120) c3(neurabin-1 isoform 4) c4(194262, 194263) c5(194264, 194265, 194266, 194267, 194268, 194269, 194270, 194271, 194272, 194273) c6( ) c7(y); #c1(PPP1R9A) c2(XP_011514685) c3(neurabin-1 isoform X3) c4( ) c5(307329, 307330, 307331, 307332, 307333, 307334, 307335, 307336, 307337, 307338) c6( ) c7(y); #c1(PPP2CA) c2(NP_002706) c3(serine/threonine-protein phosphatase 2A catalytic subunit alpha isoform) c4(131314) c5(131315, 131316, 131317, 131318, 131319, 131320) c6( ) c7(bL, gh, T, bM, F, cF, jp, aW, zf, eZ, aT, gZ, aq, j, f, B, tw); #c1(PPP2CB) c2(NP_001009552) c3(serine/threonine-protein phosphatase 2A catalytic subunit beta isoform) c4(23585) c5(23586, 23587, 23588, 23589, 23590, 23591, 23592, 23593, 23594, 23595) c6( ) c7(ad, g); #c1(PPP2R1A) c2(NP_055040) c3(serine/threonine-protein phosphatase 2A 65 kDa regulatory subunit A alpha isoform) c4(178472, 178473, 178474, 178475) c5(178476, 178477, 178478, 178479, 178480, 178481, 178482, 178483, 178484, 178485, 178486, 178487, 178488, 178489, 178490, 178491, 178492, 178493, 178494, 178495, 178496, 178497, 178498, 178499) c6( ) c7(T, aA, ci, lu, cd, er, hw, aT, alj, xQ, auY, NE, aW, Y, oV, Qm, cl, t, bz, hl, m, B, jr); #c1(PPP2R1B) c2(NP_001171033) c3(serine/threonine-protein phosphatase ZA 65 kDa regulatory subunit A beta isoform isoform d) c4(69773) c5(69774, 69775, 69776, 69777, 69778, 69779, 69780) c6( ) c7(av, T, B, bE, aW, if, cj, aT, r, bB, m, qN, t, bz, q); #c1(PPP2R1B) c2(NP_001171034) c3(serine/threonine-protein phosphatase 2A 65 kDa regulatory subunit A beta isoform isoform e) c4(69781, 69782) c5(69783, 69784, 69785, 69786, 69787, 69788, 69789, 69790, 69791, 69792, 69793, 69794, 69795) c6( ) c7(av, T, B, bE, aW, if, cj, aT, r, bB, m, qN, t, bz, q); #c1(PPP2R1B) c2(NP_002707) c3(serine/threonine-protein phosphatase ZA 65 kDa regulatory subunit A beta isoform isoform a) c4(131321, 131322, 131323, 131324) c5(131325, 131326, 131327, 131328, 131329, 131330, 131331, 131332, 131333, 131334, 131335, 131336) c6( ) c7(av, T, B, bE, aW, if, cj, aT, r, bB, m, qN, t, bz, q); #c1(PPP2R1B) c2(NP_859050) c3(serine/threonine-protein phosphatase 2A 65 kDa regulatory subunit A beta isoform isoform b) c4(255381) c5(255382, 255383, 255384, 255385, 255386, 255387) c6( ) c7(av, T, B, bE, aW, if, cj, al, r, bB, m, qN, t, bz, q); #c1(PPP2R1B) c2(NP_859051) c3(serine/threonine-protein phosphatase 2A 65 kDa regulatory subunit A beta isoform isoform c) c4(255388) c5(255389, 255390, 255391, 255392, 255393, 255394) c6( ) c7(av, T, B, bE, aW, if, cj, aT, r, bB, m, qN, t, bz, q); #c1(PPP2R2A) c2(NP_001171062) c3(serine/threonine-protein phosphatase 2A 55 kDa regulatory subunit B alpha isoform isoform 2) c4(69796) c5(69797, 69798, 69799, 69800, 69801, 69802) c6( ) c7(aE, eM, vT, aW, r, al, cz, v, kp, aNc, sD, q, ad); #c1(PPP2R2A) c2(NP_002708) c3(serine/threonine-protein phosphatase 2A 55 kDa regulatory subunit B alpha isoform isoform 1) c4(131337) c5(131338, 131339, 131340, 131341, 131342, 131343) c6( ) c7(aE, eM, vT, aW, r, al, cz, v, kp, aNc, sD, q, ad); #c1(PPP2R2B) c2(NP_001258828) c3(serine/threonine-protein phosphatase 2A 55 kDa regulatory subunit B beta isoform isoform g) c4(96570) c5(96571, 96572, 96573, 96574, 96575, 96576) c6( ) c7(EQ, rf, GC, n, j, cum, cd, PW, Gt, aT, yZ, K, akr, he, af, bA, cul, aW, RI, cm, bz, wT, alT, ky, kB, U); #c1(PPP2R2B) c2(NP_001258829) c3(serine/threonine-protein phosphatase ZA 55 kDa regulatory subunit B beta isoform isoform f) c4(96577) c5(96578, 96579, 96580, 96581, 96582, 96583, 96584) c6( ) c7(EQ, rf, GC, n, j, cum, cd, PW, Gt, aT, yZ, K, akr, he, af, bA, cul, aW, RI, cm, bz, wT, alT, ky, kB, U); #c1(PPP2R2B) c2(NP_001258877) c3(serine/threonine-protein phosphatase 2A 55 kDa regulatory subunit B beta isoform isoform d) c4(96636) c5(96637, 96638, 96639, 96640, 96641, 96642, 96643, 96644, 96645, 96646) c6( ) c7(EQ, rf, GC, n, j, cum, cd, PW, Gt, aT, yZ, K, akr, he, af, bA, cul, aW, RI, cm, bz, wT, aU, ky, KB, U); #c1(PPP2R2B) c2(NP_858060) c3(serine/threonine-protein phosphatase 2A 55 kDa regulatory subunit B beta isoform isoform a) c4(255340) c5(255341, 255342, 255343, 255344, 255345, 255346, 255347, 255348, 255349, 255350, 255351, 255352, 255353, 255354, 255355, 255356, 255357, 255358, 255359, 255360, 255361, 255362, 255363) c6( ) c7(EQ, rf, GC, n, j, cum, cd, PW, Gt, aT, yZ, K, akr, he, af, bA, cul, aW, RI, cm, bz, wT, alT, ky, kB, U); #c1(PPP2R2B) c2(NP_858061) c3(serine/threonine-protein phosphatase 2A 55 kDa regulatory subunit B beta isoform isoform e) c4(255364) c5(255365) c6( ) c7(EQ, rf, GC, n, j, cum, cd, PW, Gt, aT, yZ, K, akr, he, af, bA, cul, aW, RI, cm, bz, wT, aU, ky, KB, U); #c1(PPP2R2B) c2(NP_858062) c3(serine/threonine-protein phosphatase 2A 55 kDa regulatory subunit B beta isoform isoform b) c4(255366) c5(255367, 255368, 255369, 255370, 255371, 255372) c6( ) c7(EQ, rf, GC, n, j, cum, cd, PW, Gt, aT, yZ, K, akr, he, af, bA, cul, aW, RI, cm, bz, wT, alT, ky, KB, U); #c1(PPP2R2C) c2(NP_001193924) c3(protein phosphatase 2, regulatory subunit B, gamma isoform c) c4(83170) c5(83171, 83172, 83173, 83174, 83175, 83176, 83177, 83178, 83179, 83180) c6( ) c7(bL, ae, T, dB, Dd, fq, j, bz, sD, eA, ad); #c1(PPP2R2C) c2(NP_001193925) c3(protein phosphatase 2, regulatory subunit B, gamma isoform d) c4(83181) c5(83182, 83183, 83184, 83185, 83186, 83187) c6( ) c7(bL, ae, T, dB, Dd, fq, j, bz, sD, eA, ad); #c1(PPP2R2C) c2(NP_065149) c3 (protein phosphatase 2, regulatory subunit B, gamma isoform a) c4(204262) c5(204263, 204264, 204265, 204266, 204267, 204268) c6( ) c7(bL, ae, T, dB, Dd, fq, j, bz, sD, eA, ad); #c1(PPP2R2C) c2(NP_870991) c3(protein phosphatase 2, regulatory subunit B, gamma isoform b) c4(255906) c5(255907, 255908, 255909, 255910, 255911, 255912) c6( ) c7(bL, ae, T, dB, Dd, fq, j, bz, sD, eA, ad); #c1(PPP2R3A) c2(NP_001177376) c3(serine/threonine-protein phosphatase 2A regulatory subunit B″ subunit alpha isoform 3) c4( ) c5(72722, 72723, 72724, 72725, 72726) c6( ) c7(kp); #c1(PPP2R3A) c2(NP_002709) c3(serine/threonine-protein phosphatase 2A regulatory subunit B″ subunit alpha isoform 1) c4(131344, 131345) c5(131346, 131347, 131348, 131349, 131350, 131351, 131352) c6( ) c7(kp); #c1(PPP2R3A) c2(NP_871626) c3(serine/threonine-protein phosphatase 2A regulatory subunit B″ subunit alpha isoform 2) c4(255940) c5(255941, 255942, 255943, 255944, 255945, 255946) c6( ) c7(kp); #c1(PPP2R3A) c2(XP_011511259) c3(serine/threonine-protein phosphatase 2A regulatory subunit B″ subunit alpha isoform X2) c4(301743) c5(301744, 301745, 301746, 301747, 301748, 301749) c6( ) c7(kp); #c1(PPP2R3B) c2(NP_037371) c3(serine/threonine-protein phosphatase 2A regulatory subunit B″ subunit beta) c4( ) c5(175092, 175093, 175094, (75095, 175096) c6( ) c7(vi); #c1(PPP2R4) c2(NP_001180326) c3(serine/threonine-protein phosphatase 2A activator isoform e) c4( ) c5(73833, 73834, 73835, 73836, 73837) c6( ) c7(bL, gh, T, bM, jh, aA, bB, n, dG, Kh, ci, V, q, cd, ad, er, av, Hx, F, aE, zf, aT, h, bK, K, j, ae, af, cp, afu, xQ, kl, cul, iU, aW, aml, Y, eZ, r, af, pm, t, cm, bz, ik, bw, LB, ck, bP, c, bE, U, cj, at, m, bt, KS, B); #c1(PPP2R4) c2(NP_001258761) c3(serine/threonine-protein phosphatase 2A activator isoform f) c4( ) c5(96506, 96507, 96508, 96509, 96510) c6( ) c7(bL, gh, T, bM, jh, aA, bB, n, dG, Kh, ci, V, q, cd, ad, er, av, Hx, F, aE, zf, aT, h, bK, K, j, ae, af, cp, afu, xQ, kl, cul, iU, aW, aml, Y, eZ, r, aF, pm, t, cm, bz, ik, bw, LB, ck, bP, c, bE, U, cj, at, m, bt, KS, B); #c1(PPP2R4) c2(NP_066954) c3(serine/threonine-protein phosphatase ZA activator isoform b) c4(207701, 207702) c5(207703, 207704, 207705, 207706, 207707, 207708, 207709, 207710, 207711, 207712, 207713, 207714, 207715, 207716) c6( ) c7(bL, gh, T, bM, jh, aA, bB, n, dG, Kh, ci, V, q, cd, ad, er, av, Hx, F, aE, zf, aT, h, bK, K, j, ae, af, cp, afu, xQ, kl, cul, iU, aW, aml, Y, eZ, r, aF, pm, t, cm, bz, ik, bw, LB, ck, bP, c, bE, U, cj, at, m, bt, kS, B); #c1(PPP2R4) c2(NP_821068) c3(serine/threonine-protein phosphatase 2A activator isoform a) c4(253164) c5(253165, 253166, 253167, 253168, 253169, 253170) c6( ) c7(bL, gh, T, bM, jh, aA, bB, n, dG, Kh, ci, V, q, cd, ad, er, av, Hx, F, aE, zf, aT, h, bK, K, j, ae, af, cp, afu, xQ, kl, cul, iU, aW, aml, Y, eZ, r, aF, pm, t, cm, bz, ik, bw, LB, ck, bP, c, bE, U, cj, at, m, bt, KS, B); #c1(PPP2R4) c2(NP_821070) c3(serine/threonine-protein phosphatase 2A activator isoform d) c4(253171) c5(253172, 253173, 253174, 253175, 253176) c6( ) c7(bL, gh, T, bM, jh, aA, bB, n, dG, Kh, ci, V, q, cd, ad, er, av, Hx, F, aE, zf, aT, h, bK, K, j, ae, af, cp, afu, xQ, kl, cul, iU, aW, aml, Y, eZ, r, aF, pm, t, cm, bz, ik, bw, LB, ck, bP, c, bE, U, cj, at, m, bt, kS, B); #c1(PPP2R4) c2(XP_011517136) c3(serine/threonine-protein phosphatase 24 activator isoform X1) c4(310669, 310670) c5(310671, 310672, 310673) c6( ) c7(bL, gh, T, bM, jh, aA, bB, n, dG, Kh, ci, V, q, cd, ad, er, av, Hx, F, aE, zf, aT, h, bk, K, j, ae, af, cp, afu, xQ, kl, cul, iU, aW, aml, Y, eZ, r, aF, pm, t, cm, bz, ik, bw, LB, ck, bP, c, bE, U, cj, at, m, bt, KS, B); #c1(PPP2R4) c2(XP_011517138) c3(serine/threonine-protein phosphatase 2A activator isoform X3) c4( ) c5(310674, 310675, 310676, 310677, 310678, 310679) c6( ) c7(bL, gh, T, bM, jh, aA, bB, n, dG, Kh, ci, V, q, cd, ad, er, av, Hx, F, aE, zf, aT, h, bk, K, j, ae, af, cp, afu, xQ, kl, cul, iU, aW, aml, Y, eZ, r, aF, pm, t, cm, bz, ik, bw, LB, ck, bP, c, bE, U, cj, at, m, bt, KS, B); #c1(PPP2R4) c2(XP_011517139) c3(serine/threonine-protein phosphatase 24 activator isoform X4) c4(310680) c5(310681, 310682, 310683, 310684, 310685, 310686) c6( ) c7(bL, gh, T, bM, jh, aA, bB, n, dG, Kh, ci, V, q, cd, ad, er, av, Hx, F, aE, zf, aT, h, bK, K, j, ae, af, cp, afu, xQ, kl, cul, iU, aW, aml, Y, eZ, r, aF, pm, t, cm, bz, ik, bw, LB, ck, bP, c, bE, U, cj, at, m, bt, kS, B); #c1(PPP2R4) c2(XP_011517140) c3(serine/threonine-protein phosphatase 2A activator isoform X5) c4(310687) c5(310688, 310689) c6( ) c7(bL, gh, T, bM, jh, aA, bB, n, dG, Kh, ci, V, q, cd, ad, er, av, Hx, F, aE, zf, aT, h, bk, K, j, ae, af, cp, afu, xQ, kl, cul, iU, aW, aml, Y, eZ, r, af, pm, t, cm, bz, ik, bw, LB, ck, bP, c, bE, U, cj, at, m, bt, KS, B); #c1(PPP2R5A) c2(NP_001186685) c3(serine/threonine-protein phosphatase 2A 56 kDa regulatory subunit alpha isoform isoform 2) c4(78346) c5(78347, 78348, 78349, 78350, 78351, 78352) c6( ) c7(sD, kp, cun); #c1(PPP2R5A) c2(NP_006234) c3(serine/threonine-protein phosphatase 2A 56 kDa regulatory subunit alpha isoform isoform 1) c4(163144) c5(163145, 163146, 163147, 163148, 163149, 163150, 163151, 163152, 163153, 163154) c6( ) c7(sD, kp, cun); #c1(PPP2R5B) c2(NP_006235) c3(serine/threonine-protein phosphatase 2A 56 kDa regulatory subunit beta isoform) c4(163155, 163156, 163157) c5(163158, 163159, 163160, 163161, 163162, 163163, 163164, 163165, 163166, 163167, 163168, 163169, 163170, 163171, 163172, 163173, 163174, 163175, 163176, 163177, 163178, 163179, 163180, 163181) c6( ) c7(cuo, cul, aT, RI); #c1(PPP2R5C) c2(NP_001155197) c3(serine/threonine-protein phosphatase 2A 56 kDa regulatory subunit gamma isoform isoform e) c4(61514, 61515) c5(61516, 61517, 61518, 61519, 61520, 61521, 61522, 61523, 61524, 61525, 61526, 61527) c6( ) c7(ck, T, iT, iU, cj, bt, bN); #c1(PPP2R5C) c2(NP_001155198) c3(serine/threonine-protein phosphatase 2A 56 kDa regulatory subunit gamma isoform isoform f) c4(61528) c5(61529, 61530, 61531, 61532, 61533, 61534) c6( ) c7(ck, T, iT, iU, cj, bt, bN); #c1(PPP2R5C) c2(NP_002710) c3(serine/threonine-protein phosphatase 2A 56 kDa regulatory subunit gamma isoform isoform a) c4(131353, 131354) c5(131355, 131356, 131357, 131358, 131359, 131360, 131361) c6( ) c7(ck, T, iT, iU, cj, bt, bN); #c1(PPP2R5C) c2(NP_848701) c3(serine/threonine-protein phosphatase 2A 56 kDa regulatory subunit gamma isoform isoform b) c4(254198) c5(254199, 254200, 254201, 254202, 254203, 254204) c6( ) c7(ck, T, lT, iU, cj, bt, bN); #c1(PPP2R5C) c2(NP_848702) c3(serine/threonine-protein phosphatase 2A 56 kDa regulatory subunit gamma isoform isoform c) c4(254205) c5(254206, 254207, 254208, 254209, 254210, 254211, 254212, 254213, 254214, 254215, 254216, 254217, 254218, 254219, 254220, 254221, 254222) c6( ) c7(ck, T, lT, iU, cj, bt, bN); #c1(PPP2R5C) c2(XP_005267879) c3(serine/threonine-protein phosphatase 2A 56 kDa regulatory subunit gamma isoform isoform X3) c4(281265) c5( ) c6( ) c7(ck, T, iT, iU, cj, bt, bN); #c1(PPP2R5D) c2(NP_006236) c3(serine/threonine-protein phosphatase 2A 56 kDa regulatory subunit delta isoform isoform 1) c4(163182, 163183, 163184) c5(163185, 163186, 163187, 163188, 163189, 163190, 163191, 163192, 163193, 163194, 163195, 163196, 163197, 163198, 163199, 163200, 163201, 163202) c6( ) c7(t); #c1(PPP2R5D) c2(NP_851307) c3(serine/threonine-protein phosphatase 2A 56 kDa regulatory subunit delta isoform isoform 2) c4(254520) c5(254521, 254522, 254523, 254524, 254525, 254526) c6( ) c7(t); #c1(PPP2R5D) c2(NP_851308) c3(serine/threonine-protein phosphatase 2A 56 kDa regulatory subunit delta isoform isoform 3) c4(254527) c5(254528, 254529, 254530, 254531, 254532, 254533) c6( ) c7(t); #c1(PPP2R5E) c2(NP_001269109) c3(serine/threonine-protein phosphatase 24 56 kDa regulatory subunit epsilon isoform isoform b) c4(100455) c5(100456, 100457, 100458, 100459, 100460, 100461) c6( ) c7(T, aW, oe, EQ, cj, al, r, aA, aF, bz, q); #c1(PPP2R5E) c2(NP_001269111) c3(serine/threonine-protein phosphatase 2A 56 kDa regulatory subunit epsilon isoform isoform c) c4( ) c5(100462, 100463, 100464, 100465, 100466) c6( ) c7(T, aW, oe, EQ, cj, aT, r, aA, aF, bz, q); #c1(PPP2R5E) c2(NP_006237) c3(serine/threonine-protein phosphatase 2A 56 kDa regulatory subunit epsilon isoform isoform a) c4(163203, 163204) c5(163205, 163206, 163207, 163208, 163209, 163210, 163211) c6( ) c7(T, aW, oe, EQ, cj, aT, r, aA, aF, bz, q); #c1(PPP3CA) c2(NP_000935) c3(serine/threonine-protein phosphatase 28 catalytic subunit alpha isoform isoform 1) c4(17520, 17521, 17522, 17523, 17524) c5(17525, 17526, 17527, 17528, 17529, 17530, 17531, 17532, 17533, 17534, 17535, 17536, 17537, 17538, 17539, 17540, 17541, 17542, 17543, 17544, 17545, 17546, 17547, 17548) c6( ) c7(gh, T, dD, ea, rf, aA, fd, wV, eq, ci, C, bX, fr, q, cd, ad, er, dP, ip, ft, cc, zw, nv, aT, ab, lG, cy, dL, K, an, brw, vx, aDU, ae, af, cp, lR, iZ, aW, aDV, eH, ff, r, aF, en, g, eF, t, cm, bz, ar, kT, sS, lt, eM, iT, go, lI, gZ, np, m, y, vt, bVz, B); #c1(PPP3CA) c2(NP_001124163) c3(serine/threonine-protein phosphatase 28 catalytic subunit alpha isoform isoform 2) c4(48360) c5(48361, 48362, 48363, 48364, 48365, 48366) c6( ) c7(gh, T, dD, ea, rf, aA, fd, wV, eq, ci, C, bX, fr, q, cd, ad, er, dP, ip, ft, cc, zw, nv, aT, ab, lG, cy, dL, K, an, brw, vx, aDU, ae, af, cp, lR, iZ, aW, aDV, eH, ff, r, aF, en, g, eF, t, cm, bz, ar, kT, sS, lt, eM, iT, go, ll, gZ, np, m, y, vt, bVz, B); #c1(PPP3CA) c2(NP_001124164) c3(serine/threonine-protein phosphatase 28 catalytic subunit alpha isoform isoform 3) c4(48367, 48368) c5(48369, 48370, 48371, 48372, 48373, 48374, 48375, 48376, 48377, 48378, 48379, 48380, 48381, 48382, 48383, 48384, 48385, 48386) c6( ) c7(gh, T, dD, ea, rf, aA, fd, wV, eq, ci, C, bX, fr, q, cd, ad, er, dP, ip, ft, cc, zw, nv, aT, ab, lG, cy, dL, K, an, brw, vx, aDU, ae, af, cp, lR, iZ, aW, aDV, eH, ff, r, aF, en, g, eF, t, cm, bz, ar, kT, sS, lt, eM, iT, go, ll, gZ, np, m, y, vt, bVz, B); #c1(PPP3CB) c2(NP_001135825) c3(serine/threonine-protein phosphatase 28 catalytic subunit beta isoform isoform a) c4(53363) c5(53364, 53365, 53366, 53367, 53368, 53369) c6( ) c7(bVz, si, vQ); #c1(PPP3CB) c2(NP_001135826) c3(serine/threonine-protein phosphatase 28 catalytic subunit beta isoform isoform c) c4(53370) c5(53371, 53372, 53373, 53374, 53375, 53376) c6( ) c7(bVz, si, vQ); #c1(PPP3CB) c2(NP_001276897) c3(serine/threonine-protein phosphatase 28 catalytic subunit beta isoform isoform d) c4(108562, 108563) c5(108564, 108565, 108566, 108567, 108568, 108569, 108570, 108571, 108572, 108573, 108574, 108575, 108576) c6( ) c7(bVz, si, vQ); #c1(PPP3CB) c2(NP_001276898) c3(serine/threonine-protein phosphatase 28 catalytic subunit beta isoform isoform e) c4(108577) c5(108578, 108579, 108580, 108581, 108582) c6( ) c7(bVz, si, vQ); #c1(PPP3CB) c2(NP_066955) c3(serine/threonine-protein phosphatase 28 catalytic subunit beta isoform isoform b) c4(207717, 207718) c5(207719, 207720, 207721, 207722, 207723, 207724, 207725, 207726, 207727, 207728, 207729, 207730) c6( ) c7(bVz, si, vQ); #c1(PPP3CB) c2(XP_011538223) c3(serine/threonine-protein phosphatase 28 catalytic subunit beta isoform isoform X1) c4(335821, 335822) c5(335823, 335824, 335825, 335826, 335827, 335828, 335829, 335830, 335831, 335832, 335833, 335834) c6( ) c7(bVz, si, vQ); #c1(PPP3CC) c2(NP_001230903) c3(serine/threonine-protein phosphatase 28 catalytic subunit gamma isoform isoform 1) c4(87141) c5(87142, 87143, 87144, 87145, 87146, 87147) c6( ) c7(he, sv, xa, ad); #c1(PPP3CC) c2(NP_001230904) c3(serine/threonine-protein phosphatase 28 catalytic subunit gamma isoform isoform 3) c4(87148) c5(87149, 87150, 87151, 87152, 87153, 87154) c6( ) c7(he, sv, xa, ad); #c1(PPP3CC) c2(NP_005596) c3(serine/threonine-protein phosphatase 28 catalytic subunit gamma isoform isoform 2) c4(157786) c5(157787, 157788, 157789, 157790, 157791, 157792) c6( ) c7(he, sv, xa, ad); #c1(PPP3R1) c2(NP_000936) c3(calcineurin subunit B type 1) c4(17549) c5(17550, 17551, 17552, 17553, 17554, 17555) c6( ) c7(cz, tl, j, cm, bVz, af); #c1(PPP3R2) c2(NP_671709) c3(calcineurin subunit B type 2) c4(242095, 242096) c5(242097, 242098, 242099, 242100, 242101, 242102, 242103, 242104, 242105, 242106, 242107, 242108) c6( ) c7(aw, fq, j, ac); #c1(PPP4C) c2(NP_002711) c3(serine/threonine-protein phosphatase 4 catalytic subunit isoform 1) c4(131362) c5(131363, 131364, 131365, 131366, 131367, 131368) c6( ) c7(Th, n, kp, K, bN); #c1(PPP4R1) c2(NP_001035847) c3(serine/threonine-protein phosphatase 4 regulatory subunit 1 isoform a) c4(33263, 33264) c5(33265, 33266, 33267, 33268, 33269, 33270, 33271, 33272, 33273, 33274, 33275, 33276) c6( ) c7(bX); #c1(PPP4R1) c2(NP_005125) c3(serine/threonine-protein phosphatase 4 regulatory subunit 1 isoform b) c4(153071) c5(153072, 153073, 153074, 153075, 153076, 153077) c6( ) c7(bX); #c1(PPP4R1) c2(XP_011524079) c3(serine/threonine-protein phosphatase 4 regulatory subunit 1 isoform X3) c4(318654) c5( ) c6( ) c7(bX); #c1(PPP4R1) c2(XP_011524080) c3(serine/threonine-protein phosphatase 4 regulatory subunit 1 isoform X4) c4( ) c5(318655, 318656, 318657, 318658, 318659, 318660) c6( ) c7(bX); #c1(PPP5C) c2(NP_001191213) c3(serine/threonine-protein phosphatase 5 isoform 2) c4(81107) c5(81108, 81109, 81110, 81111, 81112, 81113) c6( ) c7(xy, bux, aT, Hn, rY, Fb, bK, ae, jr, ad); #c1(PPP5C) c2(NP_006238) c3(serine/threonine-protein phosphatase 5 isoform 1) c4(163212, 163213, 163214) c5(163215, 163216, 163217, 163218, 163219, 163220, 163221, 163222, 163223, 163224, 163225, 163226, 163227, 163228, 163229, 163230, 163231, 163232, 163233, 163234) c6( ) c7(xy, bux, aT, Hn, rY, Fb, bK, ae, jr, ad); #c1(PPP6C) c2(NP_001116827) c3(serine/threonine-protein phosphatase 6 catalytic subunit isoform a) c4(43948) c5(43949, 43950, 43951, 43952, 43953, 43954, 43955) c6( ) c7(T, Y, n, hp, B, cp); #c1(PPP6C) c2(NP_001116841) c3(serine/threonine-protein phosphatase 6 catalytic subunit isoform c) c4(43980) c5(43981, 43982, 43983, 43984, 43985, 43986, 43987, 43988, 43989, 43990, 43991, 43992) c6( ) c7(T, Y, n, hp, B, cp); #c1(PPP6C) c2(NP_002712) c3(serine/threonine-protein phosphatase 6 catalytic subunit isoform b) c4(131369) c5(131370, 131371, 131372, 131373, 131374, 131375) c6( ) c7(T, Y, n, hp, B, cp); #c1(PPP6C) c2(XP_011517149) c3(serine/threonine-protein phosphatase 6 catalytic subunit isoform X1) c4(310701, 310702) c5(310703, 310704, 310705, 310706, 310707) c6( ) c7(T, Y, n, hp, B, cp); #c1(PPP6R2) c2(NP_001229828) c3(serine/threonine-protein phosphatase 6 regulatory subunit 2 isoform 2) c4( ) c5(85377, 85378, 85379, 85380, 85381) c6( ) c7(e, C); #c1(PPP6R2) c2(NP_001229829) c3(serine/threonine-protein phosphatase 6 regulatory subunit 2 isoform 4) c4(85382) c5(85383, 85384, 85385, 85386, 85387, 85388) c6( ) c7(e, C); #c1(PPP6R2) c2(NP_055493) c3(serine/threonine-protein phosphatase 6 regulatory subunit 2 isoform 3) c4( ) c5(181499, 181500, 181501, 181502, 181503) c6( ) c7(e, C); #c1(PPP6R2) c2(XP_006724497) c3(serine/threonine-protein phosphatase 6 regulatory subunit 2 isoform X16) c4( ) c5(295985, 295986, 295987, 295988, 295989) c6( ) c7(e, C); #c1(PPP6R2) c2(XP_011529024) c3(serine/threonine-protein phosphatase 6 regulatory subunit 2 isoform X3) c4(324378) c5(324379) c6( ) c7(e, C); #c1(PPP6R2) c2(XP_011529031) c3(serine/threonine-protein phosphatase 6 regulatory subunit 2 isoform X10) c4(324380) c5(324381) c6( ) c7(e, C); #c1(PPP6R2) c2(XP_011529033) c3(serine/threonine-protein phosphatase 6 regulatory subunit 2 isoform X12) c4(324382) c5(324383) c6( ) c7(e, C); #c1(PPP6R3) c2(NP_001157632) c3(serine/threonine-protein phosphatase 6 regulatory subunit 3 isoform 4) c4(62331, 62332) c5(62333, 62334, 62335, 62336, 62337, 62338, 62339, 62340, 62341, 62342, 62343, 62344) c6( ) c7(P1, aT); #c1(PPP6R3) c2(NP_001157633) c3(serine/threonine-protein phosphatase 6 regulatory subunit 3 isoform 6) c4(62345) c5(62346, 62347, 62348, 62349, 62350, 62351) c6( ) c7(PI, aT); #c1(PPP6R3) c2(NP_001157634) c3(serine/threonine-protein phosphatase 6 regulatory subunit 3 isoform 1) c4(62352) c5(62353, 62354, 62355, 62356, 62357, 62358) c6( ) c7(PI, aT); #c1(PPP6R3) c2(NP_001157635) c3(serine/threonine-protein phosphatase 6 regulatory subunit 3 isoform 2) c4(62359) c5(62360, 62361, 62362, 62363, 62364, 62365) c6( ) c7(PI, aT); #c1(PPP6R3) c2(NP_001157636) c3(serine/threonine-protein phosphatase 6 regulatory subunit 3 isoform 3) c4(62366) c5(62367, 62368, 62369, 62370, 62371, 62372) c6( ) c7(PI, aT); #c1(PPP6R3) c2(NP_060782) c3(serine/threonine-protein phosphatase 6 regulatory subunit 3 isoform 5) c4(197761) c5(197762, 197763, 197764, 197765, 197766, 197767) c6( ) c7(PI, aT); #c1(PPP6R3) c2(XP_006718675) c3(serine/threonine-protein phosphatase 6 regulatory subunit 3 isoform X9) c4(291966) c5( ) c6( ) c7(PI, aT); #c1(PPP6R3) c2(XP_006718691) c3(serine/threonine-protein phosphatase 6 regulatory subunit 3 isoform X21) c4( ) c5(291967, 291968, 291969, 291970, 291971, 291972, 291973, 291974, 291975, 291976, 291977, 291978, 291979, 291980, 291981, 291982, 291983, 291984, 291985) c6( ) c7(PI, aT); #c1(PPP6R3) c2(XP_011543440) c3(serine/threonine-protein phosphatase 6 regulatory subunit 3 isoform X6) c4( ) c5(342337, 342338, 342339, 342340, 342341, 342342, 342343, 342344, 342345) c6( ) c7(PI, aT); #c1(PPP6R3) c2(XP_011543445) c3(serine/threonine-protein phosphatase 6 regulatory subunit 3 isoform X12) c4(342346, 342347) c5( ) c6( ) c7(PI, aT); #c1(PPP6R3) c2(XP_011543449) c3(serine/threonine-protein phosphatase 6 regulatory subunit 3 isoform X17) c4(342348) c5( ) c6( ) c7(PI, aT); #c1(PPRC1) c2(NP_001275656) c3(peroxisome proliferator-activated receptor gamma coactivator-related protein 1 isoform 2) c4(106953) c5(106954, 106955, 106956, 106957, 106958, 106959) c6( ) c7(bL, qJ, T, ib, Uc, s, aD, Y, aA, h, cy, bZ, aF, K, cm, ae, ad); #c1(PPRC1) c2(NP_001275657) c3(peroxisome proliferator-activated receptor gamma coactivator-related protein 1 isoform 3) c4( ) c5(106960) c6( ) c7(bL, qJ, T, ib, Uc, s, aD, Y, aA, h, cy, bZ, aF, K, cm, ae, ad); #c1(PPRC1) c2(NP_055877) c3(peroxisome proliferator-activated receptor gamma coactivator-related protein 1 isoform 1) c4(184114) c5(184115, 184116, 184117, 184118, 184119, 184120) c6( ) c7(bL, qJ, T, ib, Uc, s, aD, Y, aA, h, cy, bZ, aF, K, cm, ae, ad); #c1(PPRC1) c2(XP_006717794) c3(peroxisome proliferator-activated receptor gamma coactivator-related protein 1 isoform X8) c4( ) c5(291368) c6( ) c7(bL, qJ, T, ib, Uc, s, aD, Y, aA, h, cy, bZ, aF, K, cm, ae, ad); #c1(PPT2) c2(NP_001191032) c3(lysosomal thioesterase PPT2 isoform a precursor) c4(80570, 80571, 80572, 80573) c5(80574, 80575, 80576, 80577, 80578, 80579, 80580, 80581, 80582, 80583, 80584, 80585, 80586, 80587, 80588, 80589, 80590, 80591, 80592, 80593, 80594, 80595, 80596, 80597, 80598, 80599, 80600, 80601, 80602, 80603, 80604, 80605, 80606, 80607, 80608, 80609, 80610, 80611, 80612, 80613, 80614, 80615, 80616, 80617, 80618, 80619, 80620, 80621, 80622, 80623, 80624, 80625, 80626, 80627, 80628, 80629, 80630, 80631, 80632, 80633, 80634, 80635, 80636, 80637, 80638, 80639, 80640, 80641, 80642, 80643, 80644, 80645, 80646, 80647, 80648, 80649, 80650, 80651, 80652, 80653, 80654, 80655, 80656, 80657, 80658, 80659, 80660, 80661, 80662, 80663, 80664, 80665, 80666, 80667, 80668, 80669, 80670, 80671, 80672, 80673, 80674, 80675, 80676, 80677, 80678, 80679, 80680, 80681, 80682, 80683, 80684, 80685, 80686, 80687, 80688, 80689, 80690, 80691, 80692, 80693, 80694, 80695, 80696, 80697, 80698, 80699, 80700, 80701, 80702, 80703, 80704, 80705, 80706, 80707, 80708, 80709, 80710, 80711, 80712, 80713, 80714, 80715, 80716, 80717, 80718, 80719, 80720, 80721, 80722, 80723, 80724, 80725, 80726, 80727, 80728, 80729, 80730, 80731, 80732, 80733, 80734, 80735, 80736, 80737, 80738, 80739, 80740, 80741, 80742, 80743, 80744, 80745, 80746, 80747, 80748, 80749, 80750, 80751, 80752, 80753, 80754, 80755, 80756, 80757, 80758, 80759, 80760, 80761, 80762, 80763, 80764, 80765, 80766, 80767, 80768, 80769, 80770, 80771, 80772, 80773, 80774, 80775, 80776, 80777, 80778, 80779, 80780, 80781, 80782, 80783, 80784, 80785, 80786, 80787, 80788, 80789, 80790, 80791, 80792, 80793, 80794, 80795, 80796, 80797, 80798, 80799, 80800, 80801, 80802, 80803, 80804, 80805, 80806, 80807, 80808, 80809, 80810, 80811, 80812, 80813, 80814, 80815, 80816, 80817, 80818, 80819, 80820, 80821, 80822, 80823, 80824, 80825, 80826, 80827, 80828, 80829, 80830, 80831, 80832, 80833, 80834, 80835, 80836, 80837, 80838, 80839, 80840, 80841, 80842, 80843, 80844, 80845, 80846, 80847, 80848, 80849, 80850, 80851, 80852, 80853, 80854, 80855, 80856, 80857, 80858) c6( ) c7(A, U, eZ, at, h, Dp, Lq, cm, y, rY, aa); #c1(PPT2) c2(NP_619731) c3(lysosomal thioesterase PPT2 isoform b precursor) c4(237105, 237106, 237107, 237108, 237109, 237110, 237111, 237112, 237113, 237114, 237115, 237116, 237117, 237118, 237119, 237120, 237121, 237122, 237123, 237124, 237125) c5(237126, 237127, 237128, 237129, 237130, 237131, 237132, 237133, 237134, 237135, 237136, 237137, 237138, 237139, 237140, 237141, 237142, 237143, 237144, 237145, 237146, 237147, 237148, 237149, 237150, 237151, 237152, 237153, 237154, 237155, 237156, 237157, 237158, 237159, 237160, 237161, 237162, 237163, 237164, 237165, 237166, 237167, 237168, 237169, 237170, 237171, 237172, 237173, 237174, 237175, 237176, 237177, 237178, 237179, 237180, 237181, 237182, 237183, 237184, 237185, 237186, 237187, 237188, 237189, 237190, 237191, 237192, 237193, 237194, 237195) c6( ) c7(A, U, eZ, at, h, Dp, Lq, cm, y, rY, aa); #c1(PPY) c2(NP_002713) c3(pancreatic prohormone preproprotein) c4( ) c5(131376, 131377, 131378, 131379, 131380, 131381) c6( ) c7(bL, T, dA, pY, jh, v, btx, ad, sO, pX, qM, ae, af, azh, oV, ahJ, CQ, t, ar, bE, bo, mf, bFb); #c1(PPY) c2(XP_011523280) c3(pancreatic prohormone isoform X1) c4(317790) c5( ) c6( ) c7(bL, T, dA, pY, jh, v, btx, ad, sO, pX, qM, ae, af, azh, oV, ahJ, CO, t, ar, bE, bo, mf, bFb); #c1(PQBP1) c2(NP_00161464) c3(polyglutamine-binding protein 1 isoform 6) c4( ) c5(65828, 65829, 65830, 65831, 65832, 65833) c6( ) c7(asp, kj, aV, rz, Ol, cup, fc, U, nf, Kh, K, nA, cP, QK, xb, aR, hq); #c1(PQBP1) c2(XP_005272629) c3(polyglutamine-binding protein 1 isoform X1) c4(284089, 284090) c5(284091, 284092, 284093, 284094, 284095, 284096, 284097, 284098, 284099, 284100, 284101, 284102, 284103, 284104, 284105, 284106, 284107, 284108, 284109) c6( ) c7(asp, kj, aV, rz, Ol, cup, fc, U, nf, Kh, K, nA, cP, QK, xb, aR, hq); #c1(PQBP1) c2(XP_011542186) c3 (polyglutamine-binding protein 1 isoform X3) c4(340627, 340628, 340629) c5(340630, 340631, 340632, 340633, 340634, 340635, 340636, 340637, 340638, 340639, 340640, 340641, 340642, 340643, 340644, 340645, 340646, 340647, 340648, 340649, 340650, 340651, 340652, 340653, 340654, 340655, 340656, 340657, 340658, 340659, 340660, 340661, 340662) c6( ) c7(asp, kj, aV, rz, Ol, cup, fc, U, nf, Kh, K, nA, cP, QK, xb, aR, hq); #c1(PQLC3) c2(NP_001269639) c3(PQ-loop repeat-containing protein 3 isoform b) c4(102355) c5(102356, 102357, 102358, 102359, 102360, 102361) c6( ) c7(aq); #c1(PQLC3) c2(NP_689604) c3(PQ-loop repeat-containing protein 3 isoform a precursor) c4(243230) c5(243231, 243232, 243233, 243234, 243235, 243236) c6( ) c7(aq); #c1(PRAC1) c2(NP_115767) c3(small nuclear protein PRAC1) c4(223867) c5(223868, 223869, 223870, 223871, 223872, 223873) c6( ) c7(ae, ad, s); #c1(PRAC2) c2(XP_011523050) c3 (putative protein PRCA2 isoform X1) c4( ) c5(317542) c6( ) c7(ae, ad); #c1(PRADC1) c2(NP_115695) c3(protease- associated domain-containing protein 1 precursor) c4(223596) c5(223597, 223598, 223599, 223600, 223601, 223602, 223603, 223604) c6(223605, 223606) c7(uF); #c1(PRAF2) c2(NP_009144) c3(PRA1 family protein 2) c4(171124) c5(171125, 171126, 171127, 171128, 171129, 171130) c6( ) c7(cm, T, eA); #c1(PRAM1) c2(NP_115528) c3(PML-RARA-regulated adapter molecule 1) c4(223087, 223088) c5(223089, 223090, 223091, 223092, 223093, 223094, 223095, 223096, 223097, 223098, 223099, 223100) c6( ) c7(ck, T, pc, iT, fd, n, j, hF, bt, kS, pi, y); #c1(PRAME) c2(NP_996837) c3(melanoma antigen preferentially expressed in tumors isoform a) c4(264686, 264687, 264688, 264689, 264690, 264691) c5(264692, 264693, 264694, 264695, 264696, 264697, 264698, 264699, 264700, 264701, 264702, 264703, 264704, 264705, 264706, 264707, 264708, 264709, 264710, 264711, 264712, 264713, 264714, 264715, 264716, 264717, 264718, 264719, 264720, 264721, 264722, 264723, 264724, 264725, 264726, 264727, 264728, 264729, 264730, 264731, 264732, 264733, 264734, 264735, 264736, 264737, 264738, 264739, 264740, 264741, 264742, 264743, 264744, 264745, 264746, 264747) c6( ) c7(T, ex, jh, fd, hy, fr, ci, hF, aHy, zn, x, fx, ib, aT, KH, eV, iY, j, jf, xQ, je, iU, et, pr, aW, Y, eH, g, bu, hf, eF, iK, bw, ck, iM, eM, au, azK, cj, k, bt, hS); #c1(PRAP1) c2(NP_001138673) c3(proline-rich acidic protein 1 isoform 2 precursor) c4(57343, 57344) c5(57345, 57346, 57347, 57348, 57349, 57350, 57351, 57352, 57353, 57354, 57355, 57356) c6( ) c7(T, dD, wu, ea, bB, eq, bRn, eA, ad, ip, sN, aT, wm, ae, hm, cY, d, LC, aW, g, cl, t, zr, bE, hL, y, bt, B); #c1(PRAP1) c2(NP_660203) c3(proline-rich acidic protein 1 isoform 1 precursor) c4(240531, 240532) c5(240533, 240534, 240535, 240536, 240537, 240538, 240539, 240540, 240541, 240542, 240543, 240544) c6( ) c7(T, dD, wu, ea, bB, eq, bRn, eA, ad, ip, sN, aT, wm, ae, hm, cY, d, LC, aW, g, cl, t, zr, bE, hL, y, bt, B); #c1(PRB1) c2(NP_005030) c3(basic salivary proline-rich protein 1 isoform 1 preproprotein) c4( ) c5(152304, 152305, 152306, 152307, 152308, 152309, 152310) c6( ) c7(pH, b, bP, BO, bE, PX, bO, jh, bB, n, pg, h, iY, K, pm, cl, i); #c1(PRB1) c2(NP_955385) c3(basic salivary proline-rich protein 1 isoform 2 preproprotein) c4(261637) c5(261638) c6( ) c7(pH, b, bP, BO, bE, PX, bO, jh, bB, n, pg, h, iY, K, pm, cl, i); #c1(PRB1) c2(NP_955386) c3(basic salivary proline-rich protein 1 isoform 3 preproprotein) c4(261639) c5(261640, 261641, 261642, 261643, 261644, 261645) c6( ) c7(pH, b, bP, BO, bE, PX, bO, jh, bB, n, pg, h, iY, K, pm, cl, i); #c1(PRB2) c2(NP_006239) c3(basic salivary proline-rich protein 2 precursor) c4(163235) c5(163236, 163237, 163238, 163239, 163240, 163241, 163242, 163243, 163244) c6( ) c7(T, aC, xu, n, j, bED, ib, bpp, cuq, aT, aaw, K, cur, DY, qk, bqB, aW, Y, ff, eH, hQ, kD, en, cl, eF, t, cm, bz, DI, bP, hL, dX, m, jQ, it); #c1(PRB3) c2(NP_006240) c3(basic salivary proline-rich protein 3 precursor) c4(163245, 163246) c5(163247, 163248, 163249, 163250, 163251, 163252, 163253, 163254, 163255, 163256) c6( ) c7(cus); #c1(PRB4) c2(NP_001248328) c3(basic salivary proline-rich protein 4 isoform 2 precursor) c4(93693) c5( ) c6( ) c7(bU); #c1(PRB4) c2(NP_002714) c3(basic salivary proline-rich protein 4 isoform 1 precursor) c4(131382) c5(131383, 131384) c6( ) c7(b); #c1(PRC1) c2(NP_001254509) c3(protein regulator of cytokinesis 1 isoform 4) c4(94583) c5(94584, 94585, 94586, 94587, 94588, 94589) c6( ) c7(LB, bL, T, sS, fM, ck, bE, aW, eL, aT, cz, bB, dL, ey, m, bz, ai, ou); #c1(PRC1) c2(NP_003972) c3(protein regulator of cytokinesis 1 isoform 1) c4(142556) c5(142557, 142558, 142559, 142560, 142561, 142562) c6( ) c7(LB, bL, T, sS, fM, ck, bE, aW, eL, aT, cz, bB, dL, ey, m, bz, ai, oU); #c1(PRC1) c2(NP_955445) c3(protein regulator of cytokinesis 1 isoform 2) c4(261717) c5(261718, 261719, 261720, 261721, 261722, 261723) c6( ) c7(LB, bL, T, sS, fM, ck, bE, aW, eL, aT, cz, bB, dL, ey, m, bz, ai, oU); #c1(PRC1) c2(XP_005255044) c3(protein regulator of cytokinesis 1 isoform X4) c4(273524) c5(273525, 273526, 273527, 273528, 273529, 273530) c6( ) c7(LB, bL, T, sS, fM, ck, bE, aW, eL, aT, cz, bB, dL, ey, m, bz, ai, oU); #c1(PRCC) c2(NP_005964) c3 (proline-rich protein PRCC) c4(160819) c5(160820, 160821, 160822, 160823, 160824, 160825) c6( ) c7(T, go, fr, ci, rP); #c1(PRCC) c2(XP_005245370) c3(proline-rich protein PRCC isoform XI) c4(266490) c5(266491, 266492, 266493, 266494, 266495, 266496) c6( ) c7(T, go, fr, ci, rP); #c1(PRCC) c2(XP_005245371) c3(proline-rich protein PRCC isoform X2) c4(266497) c5(266498, 266499, 266500, 266501, 266502, 266503) c6( ) c7(T, go, fr, ci, rP); #c1(PRCD) c2(NP_001071088) c3(progressive rod-cone degeneration protein precursor) c4(35274) c5(35275, 35276, 35277, 35278, 35279, 35280) c6( ) c7(IR, nw, cut, nC, rY, de); #c1(PRCD) c2(XP_011523486) c3(progressive rod-cone degeneration protein isoform X1) c4( ) c5(318017, 318018) c6( ) c7(IR, nw, cut, nC, rY, de); #c1(PRCP) c2(NP_005031) c3(lysosomal Pro-X carboxypeptidase isoform 1 preproprotein) c4(152311) c5(152312, 152313, 152314, 152315, 152316, 152317, 152318) c6( ) c7(T, uC, lp, dc, acS, do, ask, aT, cz, gl, h, v, Jz, j, fD, afs, anN, V, y); #c1(PRCP) c2(NP_955450) c3(lysosomal Pro-X carboxypeptidase isoform 2 preproprotein) c4(261737, 261738) c5(261739, 261740, 261741, 261742, 261743, 261744) c6( ) c7(T, uC, lp, dc, acS, do, ask, aT, cz, gl, h, v, Jz, j, fD, afs, anN, V, y); #c1(PRCP) c2(XP_005274150) c3(lysosomal Pro-X carboxypeptidase isoform X1) c4( ) c5(284964, 284965, 284966, 284967, 284968) c6( ) c7(T, uC, lp, dc, acS, do, ask, aT, cz, gl, h, v, Jz, j, fD, afs, anN, V, y); #c1(PRDM1) c2(NP_00189) c3(PR domain zinc finger protein 1 isoform 1) c4(80295, 80296, 80297, 80298) c5( ) c6( ) c7(T, fM, wu, eX, fN, fd, jh, mC, BI, pe, aT, h, eV, ji, wm, BN, aW, qY, eZ, fe, aq, lo, iV, bz, ck, bP, cj, fl, ij, y, iu); #c1(PRDM1) c2(NP_878911) c3(PR domain zinc finger protein 1 isoform 2) c4(257145) c5(257146, 257147, 257148, 257149, 257150, 257151) c6( ) c7(T, fM, wu, eX, fN, fd, jh, mC, BI, pe, aT, h, eV, ji, wm, BN, aW, qY, eZ, fe, aq, lo, iV, bz, ck, bP, cj, fl, ij, y, iu); #c1(PRDM1) c2(XP_011534364) c3(PR domain zinc finger protein 1 isoform X1) c4( ) c5(330753, 330754) c6( ) c7(T, fM, wu, eX, fN, fd, jh, mC, BI, pe, aT, h, eV, ji, wm, BN, aW, qY, eZ, fe, aq, lo, iV, bz, ck, bP, cj, fl, ij, y, iu); #c1(PRDM1) c2(XP_011534365) c3(PR domain zinc finger protein 1 isoform X3) c4( ) c5(330755, 330756, 330757, 330758, 330759, 330760, 330761, 330762, 330763, 330764, 330765, 330766, 330767, 330768, 330769) c6( ) c7(T, fM, wu, eX, fN, fd, jh, mC, BI, pe, aT, h, eV, ji, wm, BN, aW, qY, eZ, fe, aq, lo, iV, bz, ck, bP, cj, fL, ij, y, iu); #c1(PRDM10) c2(NP_064613) c3(PR domain zinc finger protein 10 isoform 1) c4(203297, 203298) c5(203299, 203300, 203301, 203302, 203303, 203304, 203305) c6( ) c7(A, j, aT); #c1(PRDM10) c2(NP_955469) c3(PR domain zinc finger protein 10 isoform 2) c4(261814) c5(261815, 261816, 261817, 261818, 261819, 261820) c6( ) c7(A, j, aT); #c1(PRDM10) c2(NP_955470) c3(PR domain zinc finger protein 10 isoform 3) c4(261821) c5(261822, 261823, 261824, 261825, 261826, 261827) c6( ) c7(A, j, aT); #c1(PRDM10) c2(NP_955471) c3(PR domain zinc finger protein 10 isoform 4) c4(261828) c5(261829, 261830, 261831, 261832, 261833, 261834) c6( ) c7(A, j, aT); #c1(PRDM10) c2(XP_011541216) c3(PR domain zinc finger protein 10 isoform X4) c4( ) c5(339854, 339855, 339856, 339857, 339858, 339859) c6( ) c7(A, j, aT); #c1(PRDM1I) c2(NP_001243625) c3(PR domain-containing protein Il isoform 2) c4(91241, 91242) c5(91243, 91244, 91245, 91246, 91247, 91248, 91249, 91250, 91251, 91252, 91253, 91254, 91255, 91256, 91257, 91258) c6( ) c7(T); #c1(PRDM13) c2(NP_067633) c3(PR domain zinc finger protein 13) c4(209103, 209104) c5(209105, 209106, 209107, 209108, 209109, 209110, 209111, 209112, 209113, 209114, 209115, 209116, 209117, 209118) c6( ) c7(an); #c1(PRDM14) c2(NP_078780) c3(PR domain zinc finger protein 14) c4(215433, 215434) c5(215435, 215436, 215437, 215438, 215439, 215440, 215441, 215442, 215443, 215444, 215445, 215446, 215447) c6( ) c7(T, aW, aT, ci, k, Ld); #c1(PRDM4) c2(NP_036538) c3(PR domain zinc finger protein 4) c4(174278, 174279) c5(174280, 174281, 174282, 174283, 174284, 174285, 174286, 174287, 174288, 174289, 174290, 174291, 174292) c6( ) c7(n, aF, aA); #c1(PRDM5) c2(NP_001287752) c3(PR domain zinc finger protein 5 isoform 2) c4(112795) c5(112796, 112797, 112798, 112799, 112800, 112801) c6( ) c7(T, s, bE, Qu, if, gX, aA, cuu, KO, bB, aF, K, ci, qN, bz, B, all); #c1(PRDM5) c2(NP_001287753) c3(PR domain zinc finger protein 5 isoform 3) c4(112802) c5(112803, 112804, 112805, 112806, 112807, 112808) c6( ) c7(T, s, bE, Qu, if, gX, aA, cuu, KO, bB, aF, K, ci, qN, bz, B, all); #c1(PRDM5) c2(NP_061169) c3(PR domain zinc finger protein 5 isoform 1) c4(199718) c5(199719, 199720, 199721, 199722, 199723, 199724, 199725) c6( ) c7(T, s, bE, Qu, if, gX, aA, cuu, KO, bB, aF, K, ci, qN, bz, B, all); #c1(PRDM5) c2(XP_005262765) c3(PR domain zinc finger protein 5 isoform X8) c4( ) c5(278088, 278089, 278090, 278091, 278092, 278093) c6( ) c7(T, s, bE, Qu, if, gX, aA, cuu, KO, bB, aF, K, ci, qN, bz, B, aDI); #c1(PRDM5) c2(XP_011529866) c3(PR domain zinc finger protein 5 isoform X3) c4(325646) c5( ) c6( ) c7(T, s, bE, Qu, if, gX, aA, cuu, KO, bB, aF, K, ci, qN, bz, B, all); #c1(PRDM5) c2(NP_001129711) c3(putative histone-lysine N-methyltransferase PRDM6) c4(52227) c5(52228, 52229, 52230, 52231, 52232, 52233, 52234) c6( ) c7(us); #c1(PRDM7) c2(NP_001091643) c3(probable histone-lysine N-methyltransferase PRDM7) c4(38532) c5(38533, 38534, 38535, 38536, 38537, 38538) c6( ) c7(cd); #c1(PRDM7) c2(XP_011521130) c3(probable histone-lysine N-methyltransferase PRDM7 isoform X3) c4( ) c5(316077, 316078, 316079) c6( ) c7(cd); #c1(PRDM7) c2(XP_011521132) c3(probable histone-lysine N-methyltransferase PRDM7 isoform X4) c4(316080) c5(316081, 316082, 316083, 316084, 316085, 316086, 316087, 316088, 316089) c6( ) c7(cd); #c1(PRDM7) c2(XP_011521133) c3(probable histone-lysine N-methyltransferase PRDM7 isoform X5) c4(316090) c5(316091, 316092, 316093, 316094, 316095, 316096, 316097, 316098, 316099, 316100, 316101) c6( ) c7(cd); #c1(PRDM8) c2(NP_064611) c3(PR domain zinc finger protein 8) c4(203265) c5(203266, 203267, 203268, 203269, 203270, 203271, 203272, 203273, 203274, 203275, 203276, 203277, 203278, 203279, 203280, 203281) c6( ) c7(yT, oe, cd); #c1(PRDM8) c2(XP_011530436) c3(PR domain zinc finger protein 8 isoform X3) c4(326302) c5(326303, 326304, 326305, 326306) c6( ) c7(yT, oe, cd); #c1(PRDM8) c2(XP_011530437) c3(PR domain zinc finger protein 8 isoform X4) c4( ) c5(326307) c6( ) c7(yT, oe, cd); #c1(PRDM8) c2(XP_011530444) c3(PR domain zinc finger protein 8 isoform X7) c4( ) c5(326308) c6( ) c7(yT, oe, cd); #c1(PRDM9) c2(NP_064612) c3(histone-lysine N-methyltransferase PRDM9) c4(203282, 203283, 203284) c5(203285, 203286, 203287, 203288, 203289, 203290, 203291, 203292, 203293, 203294, 203295, 203296) c6( ) c7(agd, gU, alC, vr, vH, EP, jr); #c1(PRDX2) c2(NP_005800) c3(peroxiredoxin-2) c4(159560, 159561) c5(159562, 159563, 159564, 159565, 159566, 159567, 159568, 159569, 159570, 159571, 159572, 159573) c6( ) c7(ka, dt, T, kh, NO, if, lH, ht, ci, jX, PJ, qN, zP, q, ad, eA, jY, hX, wa, x, ady, ku, aE, hb, rJ, ds, aT, dL, K, j, an, ae, af, eL, Yj, zQ, s, aW, Y, eH, Xg, r, aq, S, kk, t, f, bz, eF, cm, ik, dS, iR, bw, bb, jV, VJ, eM, lT, aDs, ki, kq, Vy, hR, pg, k, m, jQ, pw, ym, bt, Vj, B, y); #c1(PRDX3) c2(NP_006784) c3(thioredoxin-dependent peroxide reductase, mitochondrial isoform a precursor) c4(168038, 168039) c5(168040, 168041, 168042, 168043, 168044, 168045, 168046, 168047, 168048, 168049, 168050, 168051) c6( ) c7(T, QO, JC, Ol, if, v, qN, cd, ad, py, aoA, aT, ey, K, j, an, ae, af, bA, jf, aW, f, bz, bb, bC, y, B, hS); #c1(PRDX4) c2(NP_006397) c3(peroxiredoxin-4 precursor) c4(164681, 164682, 164683) c5(164684, 164685, 164686, 164687, 164688, 164689, 164690, 164691, 164692, 164693, 164694, 164695, 164696, 164697, 164698, 164699) c6(164700, 164701, 164702, 164703) c7(T, kl, A, eM, iT, fJ, bt, aj, mf, dG, K, g, j, J, ae, ad); #c1(PRDX4) c2(XP_005274495) c3(peroxiredoxin-4 isoform X1) c4(285090) c5(285091, 285092, 285093, 285094, 285095, 285096, 285097, 285098, 285099, 285100, 285101, 285102, 285103, 285104, 285105, 285106) c6(285107, 285108, 285109, 285110) c7(T, kl, A, eM, iT, fJ, bt, aj, mf, dG, K, g, j, J, ae, ad); #c1(PRDX5) c2(NP_036226) c3(peroxiredoxin-5, mitochondrial isoform a precursor) c4(172453) c5(172454, 172455, 172456, 172457, 172458, 172459) c6( ) c7(T, eR, dM, aWG, aA, sM, bB, lD, GC, Zh, j, qj, mF, aR, ad, ke, Vp, av, aT, hD, h, K, j, an, cb, ae, cp, aW, ev, as, dw, t, bz, dr, ahV, asE, Ye, cpE, Hp, KE, OW, iT, TJ, bE, wE, mx, gX, lF, Da, m, bee, y, kS, B, pC); #c1(PRDX5) c2(NP_857634) c3(peroxiredoxin-5, mitochondrial isoform b precursor) c4(255283) c5(255284, 255285, 255286, 255287, 255288, 255289) c6( ) c7(T, eR, dM, aWG, aA, sM, bB, lD, GC, Zh, j, qj, mF, aR, ad, ke, Vp, av, aT, hD, h, K, j, an, cb, ae, cp, aW, ev, as, dw, t, bz, dr, ahV, asE, Ye, cpE, Hp, KE, OW, iT, TJ, bE, wE, mx, gX, lF, Da, m, bee, y, KS, B, pC); #c1(PRDX5) c2(NP_857635) c3(peroxiredoxin-5, mitochondrial isoform c precursor) c4(255290) c5(255291, 255292, 255293, 255294, 255295, 255296) c6( ) c7(T, eR, dM, aWG, aA, sM, bB, lD, GC, Zh, j, qj, mF, aR, ad, ke, Vp, av, aT, hD, h, K, j, an, cb, ae, cp, aW, ev, as, dw, t, bz, dr, ahV, asE, Ye, cpE, Hp, KE, OW, iT, TJ, bE, wE, mx, gX, lF, Da, m, bee, y, kS, B, pC); #c1(PRDX6) c2(NP_004896) c3(peroxiredoxin-6) c4(150630) c5(150631, 150632, 150633, 150634, 150635, 150636) c6( ) c7(T, os, dD, qb, aWG, aw, oZ, v, Zh, j, eq, PJ, Fc, q, ad, av, cfq, aT, K, bU, ae, af, aa, DA, brg, aW, hl, r, dw, t, f, bz, Ye, bb, m, cuv, EY, B); #c1(PREB) c2(NP_037520) c3 (prolactin regulatory element-binding protein) c4(175973) c5(175974, 175975, 175976, 175977, 175978, 175979, 175980, 175981, 175982, 175983) c6( ) c7(EW, cuw); #c1(PRELP) c2(NP_958505) c3(prolargin precursor) c4(262131) c5(262132, 262133, 262134, 262135, 262136, 262137) c6( ) c7(y, cz, d, ip, cj); #c1(PREP) c2(NP_002717) c3(prolyl endopeptidase) c4(131385) c5(131386, 131387, 131388, 131389, 131390, 131391) c6( ) c7(bL, btY, aA, bS, j, ct, cux, aoj, eA, K, H, A, mR, dN, hp, Z, cm, cuy, ac, dJ, cj, xg, L); #c1(PREP) c2(XP_005267101) c3(prolyl endopeptidase isoform X2) c4(280691, 280692) c5(280693, 280694, 280695, 280696, 280697, 280698, 280699, 280700, 280701, 280702, 280703, 280704) c6( ) c7(bL, btY, aA, bS, j, ct, cux, aoj, eA, K, H, A, mR, dN, hp, Z, cm, cuy, ac, dJ, cj, xg, L); #c1(PREPL) c2(NP_001035844) c3 (prolyl endopeptidase-like isoform 2) c4(33242) c5(33243, 33244, 33245, 33246, 33247, 33248) c6( ) c7(xC, xm, xA, beo, azB, Rj, aDx, ci, azC); #c1(PREPL) c2(NP_001035845) c3(prolyl endopeptidase-like isoform 3) c4(33249, 33250) c5(33251, 33252, 33253, 33254, 33255, 33256, 33257, 33258, 33259, 33260, 33261, 33262) c6( ) c7(xC, xm, xA, beo, azB, Rj, aDx, ci, azC); #c1(PREPL) c2(NP_001165084) c3(prolyl endopeptidase-like isoform 4) c4( ) c5(67285, 67286, 67287, 67288, 67289) c6( ) c7(xC, xm, xA, beo, azB, Rj, aDx, ci, azC); #c1(PREPL) c2(XP_011531502) c3(prolyl endopeptidase-like isoform X1) c4(327625, 327626) c5(327627, 327628, 327629, 327630, 327631, 327632, 327633, 327634, 327635, 327636, 327637, 327638, 327639, 327640, 327641, 327642, 327643, 327644, 327645, 327646, 327647, 327648, 327649, 327650, 327651, 327652, 327653) c6( ) c7(xC, xm, xA, beo, azB, Rj, aDx, ci, azC); #c1(PREX1) c2(NP_065871) c3(phosphatidylinositol 3,4,5-trisphosphate-dependent Rac exchanger 1 protein) c4(205975, 205976) c5(205977, 205978, 205979, 205980, 205981, 205982, 205983, 205984, 205985, 205986, 205987, 205988) c6( ) c7(bL, T, aW, Y, oe, aT, ae, Qt, ad); #c1(PREX2) c2(NP_079146) c3(phosphatidylinositol 3,4,5-trisphosphate-dependent Rac exchanger 2 protein isoform a) c4(217236, 217237) c5(217238, 217239, 217240, 217241, 217242, 217243) c6( ) c7(aC, aW, Y, aA, al, aF); #c1(PREX2) c2(XP_011515914) c3(phosphatidylinositol 3,4,5-trisphosphate-dependent Rac exchanger 2 protein isoform X1) c4( ) c5(309068, 309069, 309070, 309071, 309072, 309073) c6( ) c7(aC, aW, Y, aA, aT, aF); #c1(PRF1) c2(NP_001076585) c3(perforin-1 precursor) c4(37516, 37517, 37518, 37519) c5(37520, 37521, 37522, 37523, 37524, 37525, 37526, 37527, 37528, 37529, 37530, 37531, 37532, 37533) c6( ) c7(oO, bM, T, xS, fR, ea, atj, if, al, cl, gA, Vd, ci, ip, x, Gt, aDk, Ds, bGm, K, hu, or, zy, DY, eL, Zm, cp, aW, ih, hl, g, eF, cm, mK, yd, ly, bb, xK, eM, nB, iT, dc, mx, wB, cj, Zn, dW, k, m, Xu, hh, pJ, cZ, NK, dD, bJs, Ol, ht, NT, aDI, aj, BF, fr, nA, PJ, pM, qN, fK, eW, ft, pe, cuA, Ne, h, jH, dL, ey, bde, dd, dx, A, apo, mR, aHW, Cu, fJ, lb, CM, hQ, blc, aq, kp, ajz, iO, xO, RJ, bm, at, cuz, aAl, fl, brs, it, B, bL, Dw, fM, jx, aA, fF, bNl, v, j, C, bX, V, q, J, o, er, eA, anA, mP, WY, aT, yu, anz, j, ae, af, aU, jf, hM, bf, Ue, Y, oV, r, aaE, lo, en, aaO, t, aER, zM, f, bN, ck, alC, ac, c, alt, hc, DW, wR, aJQ, cS, oX, bt, zA, cuB, ao, rA, vi, iW, jh, wV, Jz, jE, fu, Gn, EA, hF, eq, pz, mC, ad, hX, qC, dP, hd, F, ib, ds, hD, cuC, ff, eH, cr, eZ, afr, bNZ, Ej, aF, hp, iV, bz, dR, gk, Bv, xT, afd, eB, aS, oQ, i, lf, KS, X, y); #c1(PRG2) c2(NP_001230174) c3(bone marrow proteoglycan isoform 2 preproprotein) c4(86046) c5(86047, 86048, 86049, 86050, 86051, 86052) c6( ) c7(bM, T, JZ, iW, jE, bdL, Fc, V, ad, hX, NF, aBy, aT, h, apj, pj, ae, d, Cw, aW, xs, lb, lC, eZ, aq, eE, lY, bkn, ig, pg, eo, B); #c1(PRG2) c2(NP_001289855) c3(bone marrow proteoglycan isoform 1 preproprotein) c4(114680, 114681, 114682, 114683, 114684) c5(114685, 114686, 114687, 114688, 114689, 114690, 114691, 114692, 114693, (14694) c6( ) c7(bM, T, JZ, iW, jE, bdL, Fc, V, ad, hX, NF, aBy, aT, h, apj, pj, ae, d, Cw, aW, xs, lb, lC, eZ, aq, eE, lY, bkn, ig, pg, eo, B); #c1(PRG4) c2(NP_001121180) c3 (proteoglycan 4 isoform B preproprotein) c4(45663, 45664) c5( ) c6( ) c7(agS, can, ck, cuD, bjs, bjt, aBm, cst, ai); #c1(PRG4) c2(NP_001121181) c3(proteoglycan 4 isoform C preproprotein) c4(45665) c5( ) c6( ) c7(agS, can, ck, cuD, bjs, bjt, aBm, cst, ai); #c1(PRG4) c2(NP_001121182) c3(proteoglycan 4 isoform D preproprotein) c4( ) c5(45666, 45667, 45668, 45669, 45670, 45671, 45672, 45673, 45674, 45675, 45676, 45677, 45678, 45679, 45680, 45681, 45682, 45683, 45684, 45685, 45686, 45687, 45688, 45689, 45690, 45691, 45692, 45693, 45694, 45695, 45696, 45697) c6( ) c7(agS, can, ck, cuD, bjs, bjt, aBm, cst, ai); #c1(PRG4) c2(NP_001290161) c3(proteoglycan 4 isoform E preproprotein) c4(114993) c5( ) c6( ) c7(agS, can, ck, cuD, bjs, bjt, aBm, cst, ai); #c1(PRG4) c2(NP_005798) c3(proteoglycan 4 isoform A preproprotein) c4(159552) c5( ) c6( ) c7(agS, can, ck, cuD, bjs, bjt, aBm, cst, ai); #c1(PRH1) c2(NP_001278243) c3(proline-rich protein HaeIII subfamily 1 isoform a precursor) c4( ) c5(109529, 109530, 109531, 109532, 109533, 109534, 109535, 109536) c6( ) c7(DS, os, aSz, ea, e, al, bB, aiC, ame, ur, ss, x, bpl, aE, bW, M, Yk, K, bgF, hH, eL, hm, LC, hl, cuE, YG, aUG, bw, aVc, DL, aUF, wE, ki, mx, wx, k, m, P, NK, bpH, dD, Rx, aw, fN, ez, n, ld, cuG, cd, ain, cc, ec, vC, acb, dL, wb, aBR, aWY, is, Df, Gc, Vb, OV, MX, sp, sq, QM, lb, fe, CO, se, fL, B, aMk, lp, ka, DM, AL, aZy, atB, DV, cz, sM, cR, v, j, cuJ, bqA, C, agf, eA, Yh, aT, cy, dz, bqe, qk, iU, DN, Pg, wL, lo, aDm, t, akF, cl, ac, AH, fD, cuF, gx, bqf, qb, bpk, oZ, pl, eq, mC, cuH, cul, tx, hX, F, ds, qh, qV, oj, vc, bqc, aZw, eZ, dr, ek, cuk, bE, ag, gZ, dX, aS, be, ei, jr); #c1(PRH2) c2(NP_001103683) c3(salivary acidic proline-rich phosphoprotein 1/2 precursor) c4(41908, 41909, 41910) c5(41911, 41912, 41913, 41914, 41915, 41916, 41917, 41918, 41919, 41920, 41921, 41922, 41923, 41924, 41925, 41926, 41927, 41928, 41929, 41930, 41931, 41932, 41933, 41934, 41935, 41936, 41937, 41938, 41939, 41940, 41941, 41942, 41943) c6( ) c7(DS, os, aSz, ea, e, al, bB, aiC, ame, ur, ss, x, bpl, aE, bW, M, Yk, K, bgF, hH, eL, hm, LC, hl, cuE, YG, aUG, bw, aVc, DL, aUF, wE, ki, mx, wx, k, m, P, NK, bpH, dD, Rx, aw, fN, ez, n, ld, cuG, cd, ain, cc, ec, vC, acb, dL, wb, aBR, aWY, is, Df, Gc, Vb, OV, MX, sp, sq, QM, lb, fe, CO, se, fL, B, aMk, lp, ka, DM, AL, aZy, atB, DV, cz, sM, cR, v, j, cuJ, bqA, C, aqf, eA, Yh, aT, cy, dz, bqe, qk, iU, DN, Pg, wL, lo, aDm, t, akF, cl, ac, AH, fD, cuF, gx, bqf, qb, bpk, oZ, pl, eq, mC, cuH, cul, tX, hX, F, ds, qh, qV, oj, vc, bqc, aZw, eZ, dr, ek, cuk, bE, ag, gZ, dX, aS, be, ei); #c1 (PRICKLE1) c2(NP_001138354) c3(prickle-like protein 1) c4(56271, 56272, 56273, 56274) c5(56275, 56276, 56277, 56278, 56279, 56280, 56281, 56282, 56283, 56284, 56285, 56286, 56287, 56288, 56289, 56290, 56291, 56292, 56293, 56294, 56295, 56296, 56297, 56298, 56299, 56300, 56301, 56302, 56303, 56304, 56305, 56306, 56307, 56308, 56309, 56310, 56311, 56312, 56313, 56314) c6( ) c7(bL, agd, iR, aAT, ha, aq, yU, yZ, bVr, il, agT, B); #c1(PRICKLE2) c2(NP_942559) c3(prickle-like protein 2) c4(260552) c5(260553, 260554, 260555, 260556, 260557) c6( ) c7(af, pN, fe, aur, aq, cul, bN); #c1(PRICKLE2) c2(XP_011531737) c3 (prickle-like protein 2 isoform X4) c4( ) c5(327825) c6( ) c7(af, pN, fe, aur, aq, cul, bN); #c1(PRICKLE2) c2(XP_011531741) c3(prickle-like protein 2 isoform X5) c4(327826) c5( ) c6( ) c7(af, pN, fe, aur, aq, cul, bN); #c1(PRICKLE2) c2(XP_011531742) c3 (prickle-like protein 2 isoform X7) c4( ) c5(327827, 327828, 327829, 327830, 327831, 327832) c6( ) c7(af, pN, fe, aur, aq, cul, bN); #c1(PRICKLE4) c2(NP_037529) c3(prickle-like protein 4) c4(176033, 176034, 176035, 176036, 176037) c5(176038, 176039, 176040, 176041, 176042, 176043, 176044, 176045, 176046, 176047, 176048, 176049, 176050, 176051, 176052, 176053, 176054, 176055, 176056, 176057, 176058, 176059, 176060, 176061, 176062, 176063, 176064, 176065, 176066, 176067) c6( ) c7(aW, aT); #c1(PRIM1) c2(NP_000937) c3(DNA primase small subunit) c4(17556, 17557) c5(17558, 17559, 17560, 17561, 17562, 17563, 17564, 17565, 17566, 17567, 17568, 17569) c6( ) c7(gv); #c1(PRIMA1) c2(XP_011544946) c3(proline-rich membrane anchor 1 isoform X1) c4(343921, 343922) c5(343923, 343924, 343925, 343926, 343927, 343928, 343929, 343930, 343931, 343932, 343933, 343934, 343935, 343936, 343937, 343938) c6( ) c7(T, ft, hd, nB, aW, Y, xs, bO, aT, fd, bK, eA, bt, bz, q, dx); #c1(PRIMPOL) c2(NP_689896) c3(DNA-directed primase/polymerase protein isoform 1) c4(244283) c5(244284, 244285, 244286, 244287, 244288, 244289) c6( ) c7(cuM); #c1(PRKAA1) c2(NP_006242) c3(5′-AMP-activated protein kinase catalytic subunit alpha-1 isoform 1) c4(163257) c5(163258, 163259, 163260, 163261, 163262, 163263) c6( ) c7(bL, T, dD, hl, if, oA, aA, bB, aj, v, j, ci, eq, qN, fr, hF, eJ, J, ad, hX, x, cc, aE, NS, aT, cy, vc, jy, ji, K, ae, af, aiV, iU, aW, Y, ff, fc, dq, aF, app, en, t, bz, ck, cj, gZ, mf, m, k, cP, ei, adh, bt, X); #c1(PRKAA1) c2(NP_996790) c3(5′-AMP-activated protein kinase catalytic subunit alpha-isoform 2) c4(264540) c5(264541, 264542, 264543, 264544, 264545, 264546) c6( ) c7(bL, T, dD, hl, if, oA, aA, bB, aj, v, j, ci, eq, qN, fr, hF, eJ, J, ad, hX, x, cc, aE, NS, aT, cy, vc, jy, ji, K, ae, af, aiV, iU, aW, Y, ff, fc, dq, aF, app, en, t, bz, ck, cj, gZ, mf, m, k, cP, ei, adh, bt, X); #c1(PRKAB1) c2(XP_005253966) c3(5′-AMP-activated protein kinase subunit beta-l isoform X1) c4(272792, 272793) c5(272794, 272795, 272796, 272797, 272798, 272799, 272800, 272801, 272802, 272803, 272804, 272805, 272806, 272807, 272808, 272809) c6( ) c7(bL, T, dD, hl, aA, bB, fd, aj, v, j, ci, eq, fr, eJ, J, ad, x, cc, aE, aT, cy, vc, jy, ji, K, ae, af, aiV, iU, aW, Y, ff, fc, dq, aF, app, en, t, bz, ck, gZ, m, k, cP, ei, adh, bt, X); #c1(PRKACA) c2(NP_002721) c3(cAMP-dependent protein kinase catalytic subunit alpha isoform Calpha1) c4(131407, 131408) c5(131409, 131410, 131411, 131412, 131413, 131414, 131415, 131416, 131417, 131418) c6( ) c7(tg, s, vf, eu, aT, vg, hR, Hu, AN, k, t, ae, af, ad, YR, DB); #c1(PRKACA) c2(NP_997401) c3(cAMP-dependent protein kinase catalytic subunit alpha isoform Calpha2) c4(265507) c5(265508, 265509, 265510, 265511, 265512, 265513, 265514, 265515, 265516, 265517, 265518, 265519, 265520, 265521, 265522, 265523) c6( ) c7(tg, s, vf, eu, aT, vg, hR, Hu, AN, k, t, ae, af, ad, YR, DB); #c1(PRKACB) c2(NP_001287844) c3(cAMP-dependent protein kinase catalytic subunit beta isoform 10) c4(112999) c5(113000, 113001, 113002, 113003, 113004, 113005, 113006, 113007, 113008, 113009, 113010, 113011) c6( ) c7(T, aA, af, eu, ad, DB); #c1(PRKACB) c2(NP_001287846) c3(cAMP-dependent protein kinase catalytic subunit beta isoform 12) c4(113012) c5(113013, 113014, 113015, 113016, 113017, 113018) c6( ) c7(T, aA, af, eu, ad, DB); #c1(PRKACG) c2(NP_002723) c3(cAMP-dependent protein kinase catalytic subunit gamma) c4(131419) c5(131420, 131421, 131422, 131423, 131424, 131425) c6( ) c7(T, xu, KB, Y, alJ, K, MC, eW); #c1(PRKAG1) c2(NP_001193638) c3(5′-AMP-activated protein kinase subunit gamma-1 isoform 3) c4(82412) c5(82413, 82414, 82415, 82416, 82417, 82418) c6( ) c7(bL, aj, dD, eq); #c1(PRKAG1) c2(NP_002724) c3(5′-AMP-activated protein kinase subunit gamma-1 isoform 1) c4(131426, 131427, 131428) c5(131429, 131430, 131431, 131432, 131433, 131434, 131435) c6( ) c7(bL, aj, dD, eq); #c1(PRKAG1) c2(XP_005269076) c3(5′-AMP-activated protein kinase subunit gamma-1 isoform X2) c4( ) c5(282293) c6( ) c7(bL, aj, dD, eq); #c1(PRKAG1) c2(XP_005269077) c3(5′-AMP-activated protein kinase subunit gamma-1 isoform X4) c4( ) c5(282294, 282295, 282296, 282297, 282298) c6( ) c7(bL, aj, dD, eq); #c1(PRKAG1) c2(XP_011536864) c3(5′-AMP-activated protein kinase subunit gamma-l isoform X1) c4( ) c5(334560, 334561, 334562, 334563, 334564) c6( ) c7(bL, aj, dD, eq); #c1(PRKAG2) c2(NP_001035723) c3(5′-AMP-activated protein kinase subunit gamma-2 isoform c) c4( ) c5(32987, 32988, 32989, 32990, 32991) c6( ) c7(bL, cuO, dD, eR, mr, wL, oe, e, Am, cuP, aj, afv, ada, eq, adb, J, cd, cc, cuN, pN, NS, adl, oj, KN, aT, cy, vc, bK, ca, bQx, fb, aW, Y, aXY, Bw, tv, dq, tu, f, cuQ, dr, acY, X, mx, wR, dy, adh, bD, B); #c1(PRKAG2) c2(NP_001291456) c3(5′-AMP-activated protein kinase subunit gamma-2 isoform d) c4(116102) c5(116103, 116104, 116105, 116106, 116107, 116108) c6( ) c7(bL, cuO, dD, eR, mr, wL, oe, e, Am, cuP, aj, afv, ada, eq, adb, J, cd, cc, cuN, pN, NS, adl, oj, KN, aT, cy, vc, bK, ca, bQx, fb, aW, Y, aXY, Bw, tv, dq, tu, f, cuQ, dr, acY, X, mx, wR, dy, adh, bD, B); #c1(PRKAG2) c2(NP_001291460) c3(5′-AMP-activated protein kinase subunit gamma-2 isoform e) c4( ) c5(116109, 116110, 116111, 116112, 116113, 116114) c6( ) c7(bL, cuO, dD, eR, mr, wL, De, e, Am, cuP, aj, afv, ada, eq, adb, J, cd, cc, cuN, pN, NS, adl, oj, KN, aT, cy, vc, bK, ca, bQx, fb, aW, Y, aXY, Bw, tv, dq, tu, f, cull, dr, acY, X, mx, wR, dy, adh, bD, B); #c1(PRKAG2) c2(NP_057287) c3(5′-AMP-activated protein kinase subunit gamma-2 isoform a) c4(189867, 189868, 189869) c5(189870, 189871, 189872, 189873, 189874, 189875, 189876) c6( ) c7(bL, cuO, dD, eR, mr, wL, oe, e, Am, cuP, aj, afv, ada, eq, adb, J, cd, cc, cuN, pN, NS, adl, oj, KN, aT, cy, vc, bK, ca, bQx, fb, aW, Y, aXY, Bw, tv, dq, tu, f, cuQ, dr, acY, X, mx, wR, dy, adh, bD, B); #c1(PRKAG3) c2(NP_059127) c3(5′-AMP-activated protein kinase subunit gamma-3) c4(193163, 193164, 193165, 193166) c5(193167, 193168, 193169, 193170, 193171, 193172, 193173, 193174, 193175, 193176, 193177, 193178, 193179, 193180, 193181, 193182, 193183, 193184, 193185, 193186, 193187, 193188, 193189, 193190, 193191, 193192, 193193, 193194) c6( ) c7(bL, dD, kl, W, nU, cuQ, aZ, aj, eq, af); #c1(PRKAR1A) c2(NP_001263218) c3(cAMP-dependent protein kinase type I-alpha regulatory subunit isoform a) c4(97043) c5(97044, 97045, 97046, 97047, 97048, 97049, 97050, 97051, 97052, 97053, 97054, 97055, 97056, 97057, 97058, 97059, 97060, 97061, 97062, 97063, 97064, 97065) c6( ) c7(bDV, T, aC, vf, fd, Ja, ci, cuS, tg, aeO, vg, iv, hu, cull, eL, yo, avH, aeR, PP, aW, Ls, cm, ahV, qJ, nB, mx, cj, iS, OD, m, OF, ht, eW, b, no, jz, dL, YI, coT, OX, iw, aq, Hu, cuT, Wt, kT, HR, HB, AN, B, aDo, dA, aA, v, V, q, eA, lg, ctx, aT, j, ae, af, wr, s, oV, Y, r, lo, bu, t, ck, nM, yl, gT, bt, gV, pY, qb, jh, oH, atN, ad, hd, bri, hP, hG, aF, bF, OG, Ew, MJ, MC); #c1(PRKAR1B) c2(NP_002726) c3(cAMP-dependent protein kinase type I-beta regulatory subunit) c4(131436, 131437, 131438, 131439) c5(131440, 131441, 131442, 131443, 131444, 131445, 131446, 131447, 131448, 131449, 131450, 131451, 131452, 131453, 131454, 131455, 131456, 131457, 131458, 131459, 131460, 131461) c6( ) c7(HB, bb, U); #c1(PRKAR1B) c2(XP_005249866) c3(cAMP-dependent protein kinase type I-beta regulatory subunit isoform X3) c4( ) c5(269919, 269920, 269921, 269922, 269923, 269924, 269925, 269926, 269927, 269928, 269929, 269930, 269931, 269932, 269933, 269934, 269935, 269936) c6( ) c7(HB, bb, U); #c1(PRKAR1B) c2(XP_011513749) c3(cAMP-dependent protein kinase type I-beta regulatory subunit isoform X2) c4(306092) c5(306093, 306094, 306095, 306096, 306097, 306098) c6( ) c7(HB, bb, U); #c1(PRKAR2A) c2(NP_004148) c3(cAMP-dependent protein kinase type Il-alpha regulatory subunit) c4(143863) c5(143864, 143865, 143866, 143867, 143868, 143869, 143870, 143871, 143872, 143873) c6( ) c7(yn); #c1(PRKAR2A) c2(XP_005265371) c3(cAMP-dependent protein kinase type Il-alpha regulatory subunit isoform X2) c4(279465) c5(279466, 279467, 279468, 279469, 279470, 279471) c6( ) c7(yn); #c1(PRKAR2B) c2(NP_002727) c3(cAMP-dependent protein kinase type II-beta regulatory subunit) c4(131462, 131463) c5(131464, 131465, 131466, 131467, 131468, 131469, 131470, 131471, 131472, 131473, 131474, 131475, 131476, 131477, 131478, 131479) c6( ) c7(v, kl, jz, eZ); #c1(PRKCA) c2(NP_002728) c3(protein kinase C alpha type) c4(131480) c5(131481, 131482, 131483, 131484, 131485, 131486) c6( ) c7(bM, T, fR, vf, dk, yk, al, aA, cz, bB, aj, dG, v, ci, C, fr, V, cd, J, ad, er, eA, av, tg, ft, iu, aE, cc, af Q, oj, aT, vg, lG, By, cy, dL, K, h, bq, he, ae, eL, eu, de, wC, yo, d, apo, s, aiV, aW, Y, ev, ff, aFr, aq, aF, Hu, en, cl, t, cm, bz, YR, es, rR, kP, eM, nB, lp, bE, hL, hR, m, ym, bt, xg, B); #c1 (PRKCA) c2(XP_011523291) c3(protein kinase C alpha type isoform X1) c4(317805) c5(317806) c6( ) c7(bM, T, FR, vf, dk, yk, al, aA, cz, bB, aj, dG, v, ci, C, fr, V, cd, J, ad, er, eA, av, tg, ft, iu, aE, cc, afQ, oj, aT, vg, lG, By, cy, dL, K, h, bq, he, ae, eL, eu, de, wC, yo, d, apo, s, aiV, aW, Y, ev, ff, aFr, aq, af, Hu, en, cl, t, cm, bz, YR, es, rR, kP, eM, nB, lp, bE, hL, hR, m, ym, bt, xg, B); #c1(PRKCA) c2(XP_011523292) c3(protein kinase C alpha type isoform X2) c4( ) c5(317807, 317808, 317809, 317810, 317811) c6( ) c7(bM, T, FR, vf, dk, yk, al, aA, cz, bB, aj, dG, v, ci, C, fr, V, cd, J, ad, er, eA, av, tg, ft, iu, aE, cc, afd, oj, aT, vg, lG, By, cy, dL, K, h, bq, he, ae, eL, eu, De, wC, yo, d, apo, s, aiV, aW, Y, ev, ff, afr, aq, aF, Hu, en, cl, t, cm, bz, YR, es, rR, kP, eM, nB, Ip, bE, hL, hR, m, ym, bt, xg, B); #c1(PRKCB) c2(NP_002729) c3(protein kinase C beta type isoform 2) c4(131487) c5(131488, 131489, 131490, 131491, 131492, 131493) c6( ) c7(bL, T, dD, fM, aC, eR, ea, dk, aw, al, lr, aA, cz, bB, aj, dG, v, ci, bO, eq, aY, mC, cd, ft, aE, Bc, nX, aT, hn, K, cbN, bq, dx, cp, wC, A, aW, Y, cr, eZ, jh, bz, op, bN, eM, lp, bE, Ny, vA, dh, X, cj, dy, fl, be, y, bt, cB, B); #c1(PRKCB) c2(NP_997700) c3(protein kinase C beta type isoform 1) c4(265702) c5(265703, 265704, 265705, 265706, 265707, 265708) c6( ) c7(bL, T, dD, fM, aC, eR, ea, dk, aw, al, lr, aA, cz, bB, aj, dG, v, ci, bO, eq, aY, mC, cd, ft, aE, Bc, nX, aT, hn, K, cbN, bq, dx, cp, wC, A, aW, Y, cr, eZ, jh, bz, op, bN, eM, lp, bE, Ny, vA, dh, X, cj, dy, fl, be, y, bt, cB, B); #c1(PRKCD) c2(NP_997704) c3(protein kinase C delta type) c4(265709, 265710) c5(265711, 265712, 265713, 265714, 265715, 265716, 265717, 265718, 265719, 265720, 265721, 265722, 265723, 265724, 265725, 265726, 265727, 265728, 265729, 265730) c6( ) c7(bL, cZ, T, aC, fG, if, cz, dG, j, ci, v, aGK, qN, AO, bgR, lR, mC, ad, eA, ip, av, ft, ib, aE, uT, Bc, pX, oj, aT, cU, cy, jy, h, K, pj, bq, dd, an, rE, cuV, ae, eu, j, de, aa, d, aAl, qt, aW, ff, Of, eZ, Og, aF, en, apx, aYo, kC, pb, iq, ck, ac, nB, bE, hk, vy, cj, fL, bt, X, cn, qc); #c1(PRKCD) c2(XP_006713320) c3(protein kinase C delta type isoform X2) c4(287083) c5(287084, 287085) c6( ) c7(bL, cZ, T, aC, fG, if, cz, dG, j, ci, v, aGK, qN, AO, bgR, lR, mC, ad, eA, ip, av, ft, ib, aE, uT, Bc, pX, oj, aT, cU, cy, jy, h, K, pj, bq, dd, an, rE, cuV, ae, eu, j, de, aa, d, aAl, qt, aW, ff, Of, eZ, Og, aF, en, apx, aYo, kC, pb, iq, ck, ac, nB, bE, hk, vy, cj, fl, bt, X, cn, qc); #c1(PRKCDBP) c2(NP_659477) c3(protein kinase C delta-binding protein) c4(240105) c5(240106, 240107, 240108, 240109, 240110, 240111, 240112) c6( ) c7(x, av, T, ac, aW, r, aT, aA, vr, cm, aF, m, k, iV, fe, t, bz, q); #c1(PRKCE) c2(XP_005264485) c3(protein kinase C epsilon type isoform X1) c4(278962) c5(278963, 278964, 278965, 278966, 278967, 278968) c6( ) c7(bM, cZ, T, fR, aw, cz, Fb, j, ci, bX, eA, ad, av, cF, cc, pN, aT, dd, bq, ae, eN, hm, aW, Y, aq, cm, bz, hq, nM, Dy, eM, nB, dB, lB, iu, ao); #c1(PRKCE) c2(XP_005264488) c3(protein kinase C epsilon type isoform X8) c4(278969) c5(278970, 278971, 278972, 278973, 278974, 278975) c6( ) c7(bM, cZ, T, FR, aw, cz, Fb, j, ci, bX, eA, ad, av, cF, cc, pN, aT, dd, bq, ae, eN, hm, aW, Y, aq, cm, bz, hq, nM, Dy, eM, nB, dB, lB, iu, ao); #c1(PRKCG) c2(NP_002730) c3(protein kinase C gamma type) c4(131494, 131495, 131496) c5(131497, 131498, 131499, 131500, 131501, 131502, 131503, 131504, 131505, 131506, 131507, 131508, 131509, 131510, 131511, 131512, 131513) c6( ) c7(KE, apo, ky, cuW, KB, al, U, zD, aU, rf, Kh, nA, akr, zB, nC, wC); #c1(PRKCH) c2(NP_006246) c3(protein kinase C eta type) c4(163264) c5(163265, 163266, 163267, 163268, 163269, 163270) c6( ) c7(dD, aA, cz, v, eq, fr, ai, cF, M, oj, eu, d, kt, aF, Z, ek, fT, wg, ac, tr, y, lB, B); #c1(PRKCH) c2(XP_011535258) c3(protein kinase C eta type isoform X3) c4(332162) c5( ) c6( ) c7(dD, aA, cz, v, eq, fr, ai, cF, M, oj, eu, d, kt, aF, Z, ek, fT, wg, ac, tr, y, lB, B); #c1(PRKCH) c2(XP_011535259) c3(protein kinase C eta type isoform X4) c4( ) c5(332163, 332164, 332165, 332166, 332167, 332168, 332169) c6( ) c7(dD, aA, cz, v, eq, fr, ai, cF, M, oj, eu, d, kt, aF, Z, ek, fT, wg, ac, tr, y, lB, B); #c1(PRKCI) c2(NP_002731) c3(protein kinase C iota type) c4(131514) c5(131515, 131516, 131517, 131518, 131519, 131520) c6( ) c7(T, ht, ci, eA, ad, abZ, bv, av, jz, aT, ji, bq, hu, ae, d, Y, ff, en, cm, t, bz, eM, Qu, m, Xu, it); #c1(PRKCA) c2(NP_001229342) c3 (protein kinase C theta type isoform 2) c4(84247) c5(84248, 84249, 84250, 84251, 84252, 84253) c6( ) c7(bL, T, fR, aw, aip, cz, cl, j, C, mC, o, ad, AW, cF, aT, jy, fb, af, A, aW, eZ, hp, ac, aSp, fl, y); #c1(PRKCQ) c2(NP_001269573) c3(protein kinase C theta type isoform 3) c4(102013) c5( ) c6( ) c7(bL, T, fR, aw, aip, cz, cl, j, C, mC, o, ad, AW, cF, aT, jy, fb, af, A, aW, eZ, hp, ac, aSp, fl, y); #c1(PRKCQ) c2(NP_001269574) c3(protein kinase C theta type isoform 4) c4( ) c5(102014, 102015, 102016, 102017, 102018, 102019, 102020, 102021, 102022, 102023, 102024, 102025) c6( ) c7(bL, T, fR, aw, aip, cz, cl, j, C, mC, o, ad, AW, cF, aT, jy, fb, af, A, aW, eZ, hp, ac, aSp, fL, y); #c1(PRKCQ) c2(XP_005252553) c3(protein kinase C theta type isoform X2) c4(271875, 271876) c5(271877) c6( ) c7(bL, T, FR, aw, aip, cz, cL, j, C, mC, o, ad, AW, cF, aT, jy, fb, af, A, aW, eZ, hp, ac, aSp, fL, y); #c1(PRKCA) c2(XP_006717528) c3 (protein kinase C theta type isoform X1) c4( ) c5(291175, 291176, 291177, 291178, 291179) c6( ) c7(bL, T, fR, aw, aip, cz, cl, j, C, mC, o, ad, AW, cF, aT, jy, fb, af, A, aW, eZ, hp, ac, aSp, fL, y); #c1(PRKCSH) c2(XP_011526433) c3(glucosidase 2 subunit beta isoform X1) c4(320737) c5(320738, 320739, 320740, 320741, 320742, 320743) c6( ) c7(T, A, aW, bll, WQ, hl, wB, adl, r, aA, aF, la, eA, il, BV, q); #c1(PRKCSH) c2(XP_011526434) c3(glucosidase 2 subunit beta isoform X2) c4(320744) c5(320745, 320746, 320747, 320748, 320749, 320750) c6( ) c7(T, A, aW, bll, WQ, hl, wB, adl, r, aA, aF, la, eA, il, BV, q); #c1(PRKCZ) c2(XP_011540075) c3(protein kinase C zeta type isoform X1) c4(338402) c5(338403, 338404, 338405, 338406, 338407, 338408, 338409, 338410, 338411, 338412) c6( ) c7(bL, T, aC, bB, mS, dG, v, fr, ad, av, iu, aE, mW, oj, aT, bK, mT, mY, mV, ae, de, mU, d, aW, ff, hl, cr, en, g, t, bz, mZ, mX, ck, es, cA, iA, bE, mf, m, fD, y, X); #c1(PRKCZ) c2(XP_011540076) c3(protein kinase C zeta type isoform X2) c4(338413) c5( ) c6( ) c7(bL, T, aC, bB, mS, dG, v, fr, ad, av, iu, aE, mW, oj, aT, bK, mT, mY, mV, ae, de, mu, d, aW, ff, hl, cr, en, g, t, bz, mZ, mX, ck, es, cA, iA, bE, mf, m, fD, y, X); #c1(PRKCZ) c2(XP_011540077) c3 (protein kinase C zeta type isoform X3) c4(338414, 338415, 338416, 338417, 338418, 338419) c5(338420, 338421, 338422, 338423, 338424, 338425, 338426, 338427) c6( ) c7(bL, T, aC, bB, mS, dG, v, fr, ad, av, iu, aE, mW, oj, aT, bK, mT, mY, mV, ae, De, mU, d, aW, ff, hl, cr, en, g, t, bz, mZ, mX, ck, es, cA, iA, bE, mf, m, fD, y, X); #c1(PRKD1) c2(NP_002733) c3(serine/threonine-protein kinase DI) c4(131528) c5(131529, 131530, 131531, 131532, 131533, 131534) c6( ) c7(bM, T, hl, vl, eR, arS, oe, aA, cz, n, fo, Gn, ci, fl, aY, q, aR, cd, ad, vN, eJ, eW, cF, aE, lg, Db, BV, arV, ayQ, aT, rX, crs, us, aBZ, K, Nj, aDZ, Xh, ae, cuX, d, iU, aW, Ls, eH, r, dq, crr, kp, aF, g, mo, eF, xw, fq, cru, dr, Qi, crv, qe, crt, wB, cuY, fP, uo, lO, aS, dy, ER, im, bD, rA, jr); #c1(PRKD2) c2(NP_001073350) c3(serine/threonine-protein kinase D2 isoform A) c4(35980) c5(35981, 35982, 35983, 35984, 35985, 35986, 35987, 35988, 35989, 35990, 35991) c6( ) c7(T, eR, arS, bSp, aA, cz, n, bB, aY, cd, ad, eA, aE, arV, pX, rx, adm, K, Nj, brV, ae, kl, d, A, iU, ff, Az, Lc, en, dr, Cv, crv, iT, bE, wB, gX, cj, aS, Hb, dy, bD, fy, BV, us); #c1(PRKD2) c2(XP_005258773) c3(serine/threonine-protein kinase D2 isoform X1) c4(275728) c5(275729, 275730, 275731, 275732, 275733) c6( ) c7(T, eR, arS, bSp, aA, cz, n, bB, aY, cd, ad, eA, aE, arV, pX, rX, adm, K, Nj, brV, ae, kl, d, A, iU, ff, Az, Lc, en, dr, Cv, crv, iT, bE, wB, gX, cj, aS, Hb, dy, bD, fy, BV, us); #c1(PRKD3) c2(XP_005264294) c3(serine/threonine-protein kinase D3 isoform X1) c4(278792) c5(278793, 278794, 278795, 278796, 278797, 278798, 278799, 278800, 278801, 278802) c6( ) c7(kP, Y, bK, ey, bX, ae, ad); #c1(PRKD3) c2(XP_011531034) c3(serine/threonine-protein kinase D3 isoform X4) c4( ) c5(326997) c6( ) c7(kP, Y, bK, ey, bX, ae, ad); #c1(PRKDC) c2(NP_001075109) c3(DNA-dependent protein kinase catalytic subunit isoform 2) c4(37239) c5(37240, 37241, 37242, 37243, 37244, 37245) c6( ) c7(T, dA, fR, if, iW, jh, aA, bB, bS, ct, ci, qN, V, q, ad, cuZ, eA, eW, av, Db, qK, oW, aT, h, dL, K, ae, eL, aa, d, iU, aW, Y, CT, r, aPf, alb, aF, as, Z, ik, bz, ck, eM, nB, dB, bE, cva, cj, dy, bC, zA, B); #c1(PRKDC) c2(NP_008835) c3(DNA-dependent protein kinase catalytic subunit isoform 1) c4(168938) c5(168939, 168940, 168941, 168942, 168943, 168944) c6( ) c7(T, dA, fR, if, iW, jh, aA, bB, bS, ct, ci, qN, V, q, ad, cuZ, eA, eW, av, Db, qK, oW, aT, h, dL, K, ae, eL, aa, d, iU, aW, Y, CT, r, aPf, alb, aF, as, Z, ik, bz, ck, eM, nB, dB, bE, cva, cj, dy, bC, zA, B); #c1(PRKG1) c2(NP_001091982) c3(cGMP-dependent protein kinase 1 isoform 1) c4(39135, 39136) c5(39137, 39138, 39139, 39140, 39141, 39142, 39143, 39144, 39145, 39146, 39147, 39148, 39149) c6( ) c7(bM, T, oe, e, bRZ, aw, aA, cz, jh, v, j, ci, aHc, eW, br, UE, AK, lg, Bc, aT, FN, kf, K, bq, cvb, he, xa, af, na, eu, AT, s, aW, zb, aeV, aq, aF, S, pm, BG, bw, bRW, ek, ck, OG, ac, bTX, kg, iS, be, vF, lC, B); #c1(PRKG1) c2(NP_006249) c3(cGMP-dependent protein kinase 1 isoform 2) c4(163271, 163272, 163273) c5(163274, 163275, 163276, 163277, 163278, 163279) c6( ) c7(bM, T, oe, e, bRZ, aw, aA, cz, jh, v, j, ci, aHc, eW, br, UE, AK, lg, Bc, aT, FN, kf, K, bq, cvb, he, xa, af, na, eu, AT, s, aW, zb, aeV, aq, aF, S, pm, BG, bw, bRW, ek, ck, OG, ac, bTX, kg, iS, be, vF, lC, B); #c1(PRKG1) c2(XP_005270029) c3(cGMP-dependent protein kinase 1 isoform X2) c4( ) c5(283038, 283039, 283040, 283041, 283042, 283043, 283044, 283045, 283046, 283047, 283048) c6( ) c7(bM, T, De, e, bRZ, aw, aA, cz, jh, v, j, ci, aHc, eW, br, UE, AK, lg, Bc, aT, FN, kf, K, bq, cvb, he, xa, af, na, eu, AT, s, aW, zb, aeV, aq, aF, S, pm, BG, bw, bRW, ek, ck, OG, ac, bTX, kg, iS, be, vF, lC, B); #c1(PRKG1) c2(XP_011538254) c3(cGMP-dependent protein kinase 1 isoform XI) c4(335856) c5(335857, 335858, 335859, 335860, 335861, 335862) c6( ) c7(bM, T, oe, e, bRZ, aw, aA, cz, jh, v, j, ci, aHc, eW, br, UE, AK, lg, Bc, aT, FN, kf, K, bq, cvb, he, xa, af, na, eu, AT, s, aW, zb, aeV, aq, aF, S, pm, BG, bw, bRW, ek, ck, OG, ac, bTX, kg, iS, be, vF, lC, B); #c1(PRKG2) c2(NP_001269410) c3(cGMP-dependent protein kinase 2 isoform b) c4( ) c5(101595, 101596, 101597, 101598, 101599) c6( ) c7(nH, nD, dz, Hr, yR); #c1(PRKG2) c2(NP_001269414) c3(cGMP-dependent protein kinase 2 isoform e) c4(101600) c5(101601, 101602, 101603, 101604, 101605, 101606) c6( ) c7(nH, nD, dz, Hr, yR); #c1(PRKG2) c2(NP_006250) c3(CGMP-dependent protein kinase 2 isoform a) c4(163280, 163281) c5(163282, 163283, 163284, 163285, 163286, 163287, 163288, 163289, 163290, 163291, 163292) c6( ) c7(nH, nD, dz, Hr, yR); #c1(PRKRA) c2(NP_001132989) c3(interferon-inducible double-stranded RNA-dependent protein kinase activator A isoform 2) c4(52801) c5(52802, 52803, 52804, 52805, 52806, 52807, 52808) c6( ) c7(vE, aj, nA, V, eW, J, Wk, cvd, aU, K, bq, afu, kj, iE, ff, bYC, Az, bcD, akt, lo, en, g, t, cvc); #c1(PRKRA) c2(NP_001132990) c3(interferon-inducible double-stranded RNA-dependent protein kinase activator A isoform 3) c4( ) c5(52809, 52810, 52811, 52812, 52813) c6( ) c7(vE, aj, nA, V, eW, J, Wk, cvd, aU, K, bq, afu, kj, iE, ff, bYC, Az, bcD, akt, lo, en, g, t, cvc); #c1(PRKRA) c2(NP_003681) c3(interferon-inducible double-stranded RNA-dependent protein kinase activator A isoform 1) c4(140168, 140169, 140170) c5(140171, 140172, 140173, 140174, 140175, 140176, 140177) c6( ) c7(vE, aj, nA, V, eW, J, Wk, cvd, aU, K, bq, afu, kj, iE, ff, bYC, Az, bcD, akt, lo, en, g, t, cvc); #c1(PRKRIR) c2(NP_004696) c3(52 kDa repressor of the inhibitor of the protein kinase) c4(148877, 148878) c5(148879, 148880, 148881, 148882, 148883, 148884, 148885, 148886, 148887, 148888, 148889, 148890) c6( ) c7(t, cd); #c1(PRKX) c2(NP_005035) c3(cAMP-dependent protein kinase catalytic subunit PRKX) c4(152319) c5(152320, 152321, 152322, 152323, 152324, 152325) c6( ) c7(Y, fr, iA); #c1(PRKX) c2(XP_005274618) c3(cAMP-dependent protein kinase catalytic subunit PRKX isoform X2) c4(285245) c5(285246, 285247, 285248, 285249, 285250, 285251) c6( ) c7(Y, fr, iA); #c1(PRL) c2(NP_000939) c3(prolactin precursor) c4(17570, 17571, 17572) c5(17573, 17574, 17575, 17576, 17577, 17578, 17579, 17580, 17581, 17582, 17583, 17584) c6( ) c7(G, T, acz, cl, ci, ip, Ef, ku, il, anV, K, qM, pk, blc, yo, zQ, cve, aW, vt, vs, g, cvf, ZE, cm, vA, QF, m, bi, dD, Uc, lT, fN, n, bBS, ct, zP, JW, cd, Vs, cc, h, aAG, Dz, avP, aq, cox, zZ, op, hq, avN, cA, PI, bo, hL, hR, HB, fl, nz, B, ul, bMD, DD, Dh, jx, cz, v, j, avO, cow, AO, V, q, UE, mj, HJ, nv, sl, aql, aT, aZd, hK, rz, ae, af, bA, apL, s, cvh, oV, aeS, bcD, r, bbs, cu, cl, t, bN, ck, ov, nM, sn, kP, ac, im, gT, bt, gO, pY, Oy, aZ, iW, uG, jh, DE, bS, eq, aVy, mC, ad, dP, cvg, oW, bdM, yh, bYY, vP, aeO, qt, PE, vj, bBO, cr, eZ, Z, dr, OG, pE, ol, y, aXu); #c1(PRLH) c2(NP_056977) c3(prolactin-releasing peptide precursor) c4(188301) c5(188302, 188303, 188304, 188305, 188306, 188307, 188308) c6(188309) c7(yo, v, K); #c1(PRLHR) c2(NP_004239) c3(prolactin-releasing peptide receptor) c4(144543) c5(144544, 144545, 144546, 144547, 144548, 144549, 144550, 144551, 144552) c6( ) c7(v, oe); #c1(PRLHR) c2(XP_011538000) c3(prolactin-releasing peptide receptor isoform XI) c4( ) c5(335562) c6( ) c7(v, oe); #c1(PRLR) c2(NP_001191243) c3(prolactin receptor isoform 6 precursor) c4(81188) c5(81189, 81190, 81191, 81192, 81193, 81194, 81195, 81196, 81197, 81198) c6( ) c7(T, lT, iW, oe, al, jh, ci, avO, zP, V, ad, ip, dP, il, jL, aT, rx, Dz, rz, ae, Yl, avg, zQ, aW, sp, aeU, eZ, aq, g, zZ, HI, t, iK, bN, sn, iS, m, fL, alm, cvi, B); #c1(PRLR) c2(NP_001191244) c3(prolactin receptor isoform 2 precursor) c4(81199, 81200) c5(81201, 81202, 81203, 81204, 81205, 81206) c6( ) c7(T, lT, iW, oe, al, jh, ci, avO, zP, V, ad, ip, dP, il, jL, aT, rx, Dz, rz, ae, YI, avg, zQ, aW, sp, aeU, eZ, aq, g, zZ, HI, t, ik, bN, sn, iS, m, fL, alm, cvi, B); #c1(PRLR) c2(NP_001191245) c3(prolactin receptor isoform 3 precursor) c4(81207) c5(81208, 81209, 81210, 81211, 81212, 81213) c6( ) c7(T, lT, iW, oe, al, jh, ci, avO, zP, V, ad, ip, dP, il, jL, aT, rx, Dz, rz, ae, Yl, avg, zQ, aW, sp, aeU, eZ, aq, g, zZ, HI, t, ik, bN, sn, iS, m, fL, alm, cvi, B); #c1(PRLR) c2(NP_001191246) c3 (prolactin receptor isoform 4 precursor) c4( ) c5(81214, 81215, 81216, 81217, 81218, 81219, 81220, 81221, 81222, 81223, 81224, 81225) c6( ) c7(T, lT, iW, oe, al, jh, ci, avO, zP, V, ad, ip, dP, il, jL, aT, rx, Dz, rz, ae, Yl, avg, zQ, aW, sp, aeU, eZ, aq, g, zZ, HI, t, ik, bN, sn, iS, m, fL, alm, cvi, B); #c1(PRLR) c2(NP_001191247) c3(prolactin receptor isoform 5 precursor) c4(81226, 81227) c5(81228, 81229, 81230, 81231, 81232, 81233, 81234, 81235, 81236, 81237, 81238, 81239, 81240, 81241, 81242, 81243, 81244, 81245, 81246, 81247, 81248, 81249, 81250, 81251, 81252, 81253, 81254, 81255, 81256, 81257, 81258, 81259, 81260, 81261, 81262, 81263, 81264) c6( ) c7(T, lT, iW, oe, al, jh, ci, avO, zP, V, ad, ip, dP, il, jL, aT, rx, Dz, rz, ae, Yl, avg, zQ, aW, sp, aeU, eZ, aq, g, zZ, HI, t, ik, bN, sn, iS, m, fl, alm, cvi, B); #c1(PRLR) c2(XP_011512371) c3 (prolactin receptor isoform X1) c4(303069, 303070, 303071, 303072, 303073, 303074, 303075) c5(303076, 303077, 303078, 303079, 303080, 303081, 303082, 303083, 303084, 303085, 303086) c6( ) c7(T, lT, iW, oe, al, jh, ci, avO, zP, V, ad, ip, dP, il, jL, aT, rx, Dz, rz, ae, Yl, avg, zL, aW, sp, aeU, eZ, aq, g, zZ, HI, t, ik, bN, sn, iS, m, fL, alm, cvi, B); #c1(PRM1) c2(NP_002752) c3(sperm protamine PI) c4(131708) c5( ) c6( ) c7(vH, V, hp, gU, ND); #c1(PRM2) c2(NP_001273285) c3(protamine-2 isoform 2) c4(104624) c5(104625, 104626, 104627, 104628, 104629, 104630) c6( ) c7(vH, pt, gU, pm, ND); #c1(PRM2) c2(NP_001273286) c3(protamine-2 isoform 3) c4( ) c5(104631, 104632, 104633, 104634, 104635) c6( ) c7(vH, pt, gU, pm, ND); #c1(PRM2) c2(NP_001273287) c3(protamine-2 isoform 4) c4( ) c5(104636) c6( ) c7(vH, pt, gU, pm, ND); #c1(PRM2) c2(NP_002753) c3(protamine-2 isoform 1) c4(131709) c5( ) c6( ) c7(vH, pt, gU, pm, ND); #c1(PRM3) c2(NP_067070) c3(protamine-3) c4(208811) c5(208812, 208813, 208814, 208815, 208816, 208817) c6( ) c7(A, gU, dA, il, aW, ht, aT, dq, agm, ci, hu, f); #c1(PRMT1) c2(NP_001527) c3(protein arginine N-methyltransferase 1 isoform 1) c4(118609, 118610, 118611) c5(118612, 118613, 118614, 118615, 118616, 118617) c6( ) c7(bL, ck, T, bM, c, yw, lp, aW, bE, eM, hL, aT, aU, bB, cl, bt, bz, X, ao); #c1(PRMT1) c2(NP_938074) c3(protein arginine N-methyltransferase 1 isoform 3) c4(259610) c5(259611, 259612, 259613, 259614, 259615, 259616) c6( ) c7(bL, ck, T, bM, c, yw, lp, aW, bE, eM, hL, aT, aU, bB, cl, bt, bz, X, ao); #c1(PRMT1) c2(XP_005258901) c3(protein arginine N-methyltransferase 1 isoform X2) c4(275872, 275873) c5(275874, 275875, 275876, 275877, 275878, 275879, 275880, 275881, 275882, 275883, 275884, 275885, 275886, 275887, 275888, 275889, 275890, 275891, 275892, 275893, 275894, 275895, 275896, 275897, 275898, 275899, 275900, 275901) c6( ) c7(bL, ck, T, bM, c, yw, lp, aW, bE, eM, hL, aT, aU, bB, cl, bt, bz, X, ao); #c1(PRMT2) c2(NP_001229793) c3(protein arginine N-methyltransferase 2 isoform 2) c4(85236) c5(85237, 85238, 85239, 85240, 85241, 85242) c6( ) c7(bM, lp, aW, aT, aU, yA, f, xg); #c1(PRMT2) c2(NP_001229794) c3(protein arginine N-methyltransferase 2 isoform 3) c4(85243) c5(85244, 85245, 85246, 85247, 85248, 85249) c6( ) c7(bM, lp, aW, aT, al, yA, f, xg); #c1(PRMT2) c2(NP_001229795) c3(protein arginine N-methyltransferase 2 isoform 4) c4( ) c5(85250, 85251, 85252, 85253, 85254, 85255) c6( ) c7(bM, lp, aW, aT, al, yA, f, xg); #c1(PRMT2) c2(NP_001273606) c3(protein arginine N-methyltransferase 2 isoform 6) c4(105427, 105428) c5(105429, 105430, 105431, 105432, 105433, 105434) c6( ) c7(bM, lp, aW, aT, aU, yA, f, xg); #c1(PRMT2) c2(XP_005261168) c3(protein arginine N-methyltransferase 2 isoform X1) c4(277250) c5(277251, 277252, 277253, 277254, 277255, 277256, 277257, 277258, 277259, 277260, 277261, 277262, 277263, 277264) c6( ) c7(bM, lp, aW, aT, aU, yA, f, xg); #c1(PRMT2) c2(XP_006724061) c3(protein arginine N-methyltransferase 2 isoform X2) c4(295677) c5(295678, 295679, 295680, 295681, 295682, 295683) c6( ) c7(bM, lp, aW, aT, aU, yA, f, xg); #c1(PRMT2) c2(XP_006724063) c3(protein arginine N-methyltransferase 2 isoform X3) c4(295684) c5(295685, 295686, 295687, 295688, 295689, 295690) c6( ) c7(bM, lp, aW, al, aU, yA, f, xg); #c1(PRMT3) c2(NP_001138638) c3(protein arginine N-methyltransferase 3 isoform 2) c4(57308, 57309) c5(57310, 57311, 57312, 57313, 57314, 57315, 57316, 57317, 57318, 57319, 57320) c6( ) c7(bM, k); #c1(PRMT3) c2(NP_005779) c3(protein arginine N-methyltransferase 3 isoform 1) c4(159267, 159268) c5(159269, 159270, 159271, 159272, 159273, 159274, 159275, 159276, 159277, 159278, 159279, 159280, 159281) c6( ) c7(bM, k); #c1(PRMT5) c2(NP_001034708) c3(protein arginine N-methyltransferase 5 isoform b) c4(32104) c5(32105, 32106, 32107, 32108, 32109, 32110, 32111) c6( ) c7(ck, bM, T, iu, d, bP, zL, wu, iW, jh, cj, aA, rO, aF, eA, g, bu, as, wm, bz, cd); #c1(PRMT5) c2(NP_001269882) c3 (protein arginine N-methyltransferase 5 isoform c) c4(103016) c5(103017, 103018, 103019, 103020, 103021, 103022) c6( ) c7(ck, bM, T, iu, d, bP, zl, wu, iW, jh, cj, aA, rO, aF, eA, g, bu, as, wm, bz, cd); #c1(PRMT5) c2(NP_001269883) c3(protein arginine N-methyltransferase 5 isoform d) c4( ) c5(103023, 103024, 103025, 103026, 103027) c6( ) c7(ck, bM, T, iu, d, bP, zL, wu, iW, jh, cj, aA, rO, aF, eA, g, bu, as, wm, bz, cd); #c1(PRMT5) c2(NP_001269885) c3(protein arginine N-methyltransferase 5 isoform f) c4(103028) c5(103029, 103030, 103031, 103032, 103033, 103034) c6( ) c7(ck, bM, T, iu, d, bP, zL, wu, iW, jh, cj, aA, rO, aF, eA, g, bu, as, wm, bz, cd); #c1(PRMT5) c2(NP_006100) c3(protein arginine N-methyltransferase 5 isoform a) c4(161886, 161887) c5(161888, 161889, 161890, 161891, 161892, 161893) c6( ) c7(ck, bM, T, iu, d, bP, zL, wu, iW, jh, cj, aA, rO, af, eA, g, bu, as, wm, bz, cd); #c1(PRMT8) c2(NP_001243465) c3(protein arginine N-methyltransferase 8 isoform 2) c4(90880) c5(90881, 90882, 90883, 90884, 90885, 90886) c6( ) c7(WY, aaE, j, c); #c1(PRMT8) c2(NP_062828) c3(protein arginine N-methyltransferase 8 isoform 1) c4(202172) c5(202173, 202174, 202175, 202176, 202177, 202178) c6( ) c7(WY, aaE, j, c); #c1(PRND) c2(NP_036541) c3(prion-like protein doppel preproprotein) c4(174300) c5(174301, 174302, 174303, 174304, 174305, 174306) c6( ) c7(dt, jV, hb, eH, as, eF, af, dS); #c1(PRNP) c2(NP_001073592) c3(major prion protein preproprotein) c4(36047, 36048, 36049, 36050) c5(36051, 36052, 36053, 36054, 36055, 36056, 36057, 36058, 36059, 36060, 36061, 36062, 36063, 36064, 36065, 36066, 36067, 36068, 36069, 36070, 36071, 36072, 36073, 36074, 36075, 36076, 36077, 36078, 36079, 36080) c6( ) c7(bM, cvo, T, fR, cvm, ea, agU, l, Jd, ci, aR, jY, ku, cvn, aE, K, H, bMl, aW, ih, eF, cm, eY, bb, jV, Kv, ki, U, nw, R, cvj, dt, dD, aik, jX, aqL, rc, aJr, Sw, Hx, h, yZ, ey, bl, cvk, aPZ, kp, kk, alj, acM, Fr, ky, ql, agl, bom, Lp, at, abd, L, ka, cbW, Nt, bBX, aA, GC, v, aU, bX, il, yU, V, q, eA, PW, aaF, yw, hb, aT, cy, bK, an, af, eu, Ft, ak, abs, cvs, Y, r, lo, aaO, Mu, nC, f, wA, dS, ov, bdn, axS, rR, ac, c, kB, cvr, AC, jQ, aen, ao, kh, aal, ha, dG, uu, fu, eq, bKZ, vN, ad, hX, Dq, ds, cvl, abg, oj, cvq, ama, acQ, d, eH, rU, gl, EH, aF, EZ, cvp, dr, dR, kq, MJ, fh, CW); #c1(PRNP) c2(NP_001258490) c3(alternative prion protein isoform AltPrp) c4( ) c5(96082) c6( ) c7(bM, cvo, T, fR, cvm, ea, agU, l, Jd, ci, aR, jY, ku, cvn, aE, K, H, bMl, aW, ih, eF, cm, eY, bb, jV, Kv, ki, U, nw, R, cvj, dt, dD, aik, jX, aqL, rc, aJr, Sw, Hx, h, yZ, ey, bl, cvk, aPZ, kp, kk, alj, acM, Fr, ky, ql, agl, bom, Lp, at, abd, L, ka, cbW, Nt, bBX, aA, GC, v, aU, bX, il, yU, V, q, eA, PW, aaF, yw, hb, aT, cy, bK, an, af, eu, Ft, ak, abs, cvs, Y, r, lo, aaO, Mu, nC, f, wA, dS, ov, bdn, axS, rR, ac, c, kB, cvr, AC, jQ, aen, ao, kh, aal, ha, dG, uu, fu, eq, bKZ, vN, ad, hX, Dq, ds, cvl, abg, oj, cvq, ama, acQ, d, eH, rU, gl, EH, aF, EZ, cvp, dr, dR, kq, MJ, fh, CW); #c1(PROC) c2(NP_000303) c3(vitamin K-dependent protein C preproprotein) c4( ) c5(6724, 6725, 6726, 6727, 6728, 6729, 6730, 6731, 6732, 6733, 6734, 6735, 6736, 6737, 6738, 6739, 6740, 6741, 6742, 6743, 6744, 6745, 6746, 6747, 6748, 6749, 6750) c6(6751, 6752, 6753, 6754, 6755, 6756) c7(uB, T, aC, Fj, pf, YU, if, e, al, Yb, bB, ame, ci, YD, aE, pX, Yk, iv, gs, K, bgF, hu, eL, cvu, Qe, Ys, YL, yo, aW, LZ, cuE, YG, Yp, NR, g, cvt, zE, aaD, uC, eF, bw, iP, y, eM, nB, AM, wE, cj, cvw, iS, YP, m, YK, P, dD, Qj, ht, NT, fN, n, aj, fr, qN, eh, eW, ft, no, jz, Yc, lt, By, dL, aWY, is, he, KV, YT, pl, YI, YO, A, MD, sq, iw, ge, fe, aq, cM, Yr, hq, Ye, Yl, ar, iO, YE, bP, dB, se, hl, bm, pg, YH, it, B, DV, aA, cR, cz, wo, j, C, AO, q, J, Yh, YA, er, eJ, Yn, aT, yu, ae, jf, Uf, Yw, s, vT, Ya, Y, NE, r, lo, en, cl, t, YW, Yq, Dy, ac, VJ, nM, Li, QG, gX, LS, hS, cvv, Mb, sY, hl, Yu, df, YM, ml, jh, eq, Fc, iJ, ad, hX, av, hd, NS, Yo, qV, lC, ji, YQ, fb, iL, Yi, WW, MG, Yj, BO, qt, Fm, ff, xs, eH, Kf, hp, aF, iV, bz, dr, YR, bF, gk, bE, ag, AR, pP, aGg, MC, hE, X, jr); #c1(PROC) c2(XP_005263772) c3(vitamin K-dependent protein C isoform X1) c4(278520, 278521, 278522) c5(278523, 278524, 278525) c6( ) c7(uB, T, aC, Fj, pf, YU, if, e, al, Yb, bB, ame, ci, YD, aE, pX, Yk, iv, gs, K, bgF, hu, eL, cvu, de, Ys, YL, yo, aW, LZ, cuE, YG, Yp, NR, g, cvt, zE, aaD, uC, eF, bw, iP, y, eM, nB, AM, wE, cj, cvw, iS, YP, m, YK, P, dD, Qj, ht, NT, fN, n, aj, fr, qN, eh, eW, ft, no, jz, Yc, lt, By, dL, aWY, is, he, KV, YT, pl, YI, YO, A, MD, sq, iw, ge, fe, aq, cM, Yr, hq, Ye, Yl, ar, iO, YE, bP, dB, se, hl, bm, pg, YH, it, B, DV, aA, cR, cz, wo, j, C, A, q, J, Yh, YA, er, eJ, Yn, aT, yu, ae, jf, Uf, Yw, s, vT, Ya, Y, NE, r, lo, en, cl, t, YW, Yq, Dy, ac, VJ, nM, Li, QG, gX, LS, hS, cvv, Mb, sY, hl, Yu, df, YM, ml, jh, eq, Fc, iJ, ad, hX, av, hd, NS, Yo, qV, lC, ji, YQ, fb, iL, Yi, WW, MG, Yj, BO, qt, Fm, ff, xs, eH, Kf, hp, aF, iV, bz, dr, YR, bF, gk, bE, ag, AR, pP, aGg, MC, HE, X, jr); #c1(PROC) c2(XP_005263773) c3(vitamin K-dependent protein C isoform X2) c4(278526, 278527, 278528) c5(278529, 278530, 278531, 278532, 278533, 278534, 278535, 278536, 278537, 278538, 278539, 278540, 278541, 278542, 278543, 278544, 278545, 278546, 278547, 278548, 278549, 278550, 278551, 278552) c6(278553, 278554, 278555, 278556, 278557, 278558) c7(uB, T, aC, Fj, pf, YU, if, e, al, Yb, bB, ame, ci, YD, aE, pX, Yk, iv, gs, K, bgF, hu, eL, cvu, De, Ys, YL, yo, aW, LZ, cuE, YG, Yp, NR, g, cvt, zE, aaD, uC, eF, bw, iP, y, eM, nB, AM, wE, cj, cvw, iS, YP, m, YK, P, dD, Qj, ht, NT, fN, n, aj, fr, qN, eh, eW, ft, no, jz, Yc, lt, By, dL, aWY, is, he, KV, YT, pl, YI, YO, A, MD, sq, iw, ge, fe, aq, cM, Yr, hq, Ye, Yl, ar, iO, YE, bP, dB, se, hl, bm, pg, YH, it, B, DV, aA, cR, cz, wo, j, C, AO, q, J, Yh, YA, er, eJ, Yn, al, yu, ae, jf, Uf, Yw, s, vT, Ya, Y, NE, r, lo, en, cl, t, YW, Yq, Dy, ac, VJ, nM, Li, QG, gX, LS, hS, cvv, Mb, sY, hl, Yu, df, YM, ml, jh, eq, Fc, iJ, ad, hX, av, hd, NS, Yo, qV, lC, ji, YQ, fb, iL, Yi, WW, MG, Yj, BO, qt, Fm, ff, xs, eH, Kf, hp, aF, iV, bz, dr, YR, bF, gk, bE, ag, AR, pP, aGg, MC, HE, X, jr); #c1(PROCR) c2(NP_006395) c3(endothelial protein C receptor precursor) c4(164670) c5(164671, 164672, 164673, 164674, 164675, 164676, 164677, 164678, 164679, 164680) c6( ) c7(bL, T, jv, sQ, e, aw, oR, v, j, C, uE, eh, mC, q, rc, ib, qh, dV, kH, aT, h, wb, Vb, nO, aW, eZ, r, bpA, t, bz, aHQ, dr, ac, ig, ag, m, fl, pw, aHP, B); #c1(PRODH) c2(XP_011528580) c3 (proline dehydrogenase I, mitochondrial isoform X1) c4(323752) c5( ) c6( ) c7(T, iH, ha, aNN, bB, fr, nA, agY, agd, bK, K, he, rL, alp, eZ, jb, cvx, bN, es, eM, iA, bE, sv, hh); #c1(PRODH) c2(XP_011528581) c3(proline dehydrogenase I, mitochondrial isoform X2) c4( ) c5(323753, 323754, 323755, 323756, 323757, 323758) c6( ) c7(T, iH, ha, aNN, bB, fr, nA, agY, agd, bK, K, he, rl, alp, eZ, jb, cvx, bN, es, eM, iA, bE, sv, hh); #c1(PROK2) c2(NP_001119600) c3(prokineticin-2 isoform a precursor) c4(44275) c5(44276, 44277, 44278, 44279, 44280, 44281) c6( ) c7(aRN, acz, cvy, bO, aj, cd, yn, UE, Jb, tT, bK, avP, bUf, cvz, fe, aMP, nM, cA, aqm, aS, UF, ra); #c1(PROK2) c2(NP_068754) c3(prokineticin-2 isoform b precursor) c4(210177) c5(210178, 210179, 210180, 210181, 210182, 210183) c6( ) c7(aRN, acz, cvy, bO, aj, cd, yn, UE, Jb, tT, bK, avP, bUf, cvz, fe, aMP, nM, cA, aqm, aS, UF, ra); #c1(PROKR1) c2(NP_620414) c3(prokineticin receptor 1) c4(237607) c5(237608, 237609, 237610, 237611, 237612, 237613, 237614, 237615, 237616, 237617) c6( ) c7(gx, T, sn); #c1(PROKR2) c2(NP_658986) c3(prokineticin receptor 2) c4(239664) c5(239665, 239666, 239667, 239668, 239669, 239670, 239671, 239672, 239673, 239674) c6( ) c7(gx, T, aRN, acz, bO, Vs, yn, UE, tT, cU, bk, xa, yi, avP, bUf, aVQ, cvB, aeS, sn, cA, cvA, aqm, UF); #c1(PROLI) c2(NP_067048) c3(proline-rich protein 1 isoform 1 precursor) c4(208720) c5(208721, 208722, 208723, 208724, 208725, 208726, 208727, 208728) c6(208729, 208730) c7(T, By, aj, bz, it, J); #c1(PROM1) c2(NP_001139319) c3(prominin-1 isoform 2 precursor) c4(59163, 59164, 59165) c5(59166, 59167, 59168, 59169, 59170, 59171, 59172, 59173, 59174, 59175) c6( ) c7(ID, T, EQ, bO, bB, fd, gf, ci, aR, RL, x, bv, aE, dV, K, cvG, cp, ly, yo, JI, aW, uY, Qm, g, cvD, eF, cm, bw, Qt, cvC, nB, eM, iA, wB, cj, nw, m, ym, iu, wu, sw, zX, Or, aw, NT, n, aj, fr, Ea, eh, eW, b, ft, hw, jz, gd, bkP, By, hB, cY, PX, aq, kp, mo, cM, Ix, W, bP, asl, dB, Qu, HL, B, cvE, dA, De, nx, aA, j, q, er, qH, eA, azn, nv, aT, ae, jf, s, Ar, nO, oV, Y, ev, iU, r, en, cl, t, nC, ck, cT, kP, c, Dy, ac, np, bt, rA, VB, LS, Mb, jN, hF, iJ, ad, hX, Jm, iC, hd, py, Sr, arV, io, wm, fb, d, lR, ue, cW, ff, xs, eH, eZ, aF, iR, bz, cvF, es, bF, nb, bE, X, BA); #c1(PROM1) c2(XP_005248253) c3(prominin-1 isoform X5) c4( ) c5(268280, 268281, 268282, 268283, 268284, 268285, 268286) c6( ) c7(ID, T, EQ, bO, bB, fd, gf, ci, aR, RL, x, bv, aE, dV, K, cvG, cp, ly, yo, JI, aW, uY, Qm, g, cvD, eF, cm, bw, At, cvC, nB, eM, iA, wB, cj, nw, m, ym, iu, wu, sw, zX, Or, aw, NT, n, aj, fr, Ea, eh, eW, b, ft, hw, jz, gd, bkP, By, hB, cY, PX, aq, kp, mo, cM, lx, W, bP, asl, dB, Qu, hL, B, cvE, dA, oe, nx, aA, j, q, er, qH, eA, azn, nv, aT, ae, jf, s, Or, nO, oV, Y, ev, iU, r, en, cl, t, nC, ck, cT, kP, c, Dy, ac, np, bt, rA, VB, LS, Mb, jN, hF, iJ, ad, hX, Jm, iC, hd, py, Sr, arV, io, wm, fb, d, lR, ue, cW, ff, xs, eH, eZ, af, iR, bz, cvF, es, bF, nb, bE, X, BA); #c1(PROM1) c2(XP_011512199) c3(prominin-1 isoform X1) c4(302747) c5(302748, 302749, 302750, 302751, 302752, 302753, 302754, 302755, 302756, 302757, 302758, 302759, 302760, 302761, 302762) c6( ) c7(ID, T, EQ, bO, bB, fd, gf, ci, aR, RL, x, bv, aE, dV, K, cvG, cp, ly, yo, JI, aW, uY, Qm, g, cvD, eF, cm, bw, Qt, cvC, nB, eM, iA, wB, cj, nw, m, ym, iu, wu, sw, zX, Or, aw, NT, n, aj, fr, Ea, eh, eW, b, ft, hw, jz, gd, bkP, By, hB, cY, PX, aq, kp, mo, cM, lx, W, bP, asl, dB, Qu, hL, B, cvE, dA, De, nx, aA, j, q, er, qH, eA, azn, nv, aT, ae, jf, s, Ar, nO, oV, Y, ev, iU, r, en, cl, t, nC, ck, cT, kP, c, Dy, ac, np, bt, rA, VB, LS, Mb, jN, hF, iJ, ad, hX, Jm, iC, hd, py, Sr, arV, io, wm, fb, d, lR, ue, cW, ff, xs, eH, eZ, aF, iR, bz, cvF, es, bF, nb, bE, X, BA); #c1(PROM1) c2(XP_011512201) c3(prominin-1 isoform X7) c4(302763, 302764) c5(302765, 302766, 302767, 302768, 302769, 302770) c6( ) c7(ID, T, EQ, b, bB, fd, gf, ci, aR, RL, x, bv, aE, dV, K, cvG, cp, ly, yo, JI, aW, uY, Qm, g, cvD, eF, cm, bw, Qt, cvC, nB, eM, iA, wB, cj, nw, m, ym, iu, wu, sw, zX, Or, aw, NT, n, aj, fr, Ea, eh, eW, b, ft, hw, jz, gd, bkP, By, hB, cY, PX, aq, kp, mo, cM, lx, W, bP, asl, dB, Qu, hL, B, cvE, dA, oe, nx, aA, j, q, er, qH, eA, azn, nv, aT, ae, jf, s, Ar, nO, oV, Y, ev, iU, r, en, cl, t, nC, ck, cT, kP, c, Dy, ac, np, bt, rA, VB, LS, Mb, jN, hF, iJ, ad, hX, Jm, iC, hd, py, Sr, arV, io, wm, fb, d, lR, ue, cW, ff, xs, eH, eZ, aF, iR, bz, cvF, es, bF, nb, bE, X, BA); #c1(PROM1) c2(XP_011512202) c3(prominin-1 isoform X2) c4(302771) c5(302772, 302773, 302774, 302775, 302776, 302777) c6( ) c7(ID, T, EA, bO, bB, fd, gf, ci, aR, RL, x, bv, aE, dV, K, cvG, cp, ly, yo, JI, aW, uY, Am, g, cvD, eF, cm, bw, Qt, cvC, nB, eM, iA, wB, cj, nw, m, ym, iu, wu, sw, zX, Or, aw, NT, n, aj, fr, Ea, eh, eW, b, ft, hw, jz, gd, bkP, By, hB, cY, PX, aq, kp, mo, cM, lx, W, bP, asl, dB, Qu, hL, B, cvE, dA, oe, nx, aA, j, q, er, qH, eA, azn, nv, aT, ae, jf, s, Ar, nO, oV, Y, ev, iU, r, en, cl, t, nC, ck, cT, kP, c, Dy, ac, np, bt, rA, VB, LS, Mb, jN, hF, iJ, ad, hX, Jm, iC, hd, py, Sr, arV, io, wm, fb, d, lR, ue, cW, ff, xs, eH, eZ, aF, iR, bz, cvF, es, bF, nb, bE, X, BA); #c1(PROM1) c2(XP_011512204) c3(prominin-1 isoform X3) c4(302778) c5(302779, 302780, 302781, 302782, 302783, 302784) c6( ) c7(ID, T, EQ, bO, bB, fd, gf, ci, aR, RL, x, bv, aE, dV, K, cvG, cp, ly, yo, JI, aW, uY, Qm, g, cvD, eF, cm, bw, Qt, cvC, nB, eM, iA, wB, cj, nw, m, ym, iu, wu, sw, zX, Or, aw, NT, n, aj, fr, Ea, eh, eW, b, ft, hw, jz, gd, bkP, By, hB, cY, PX, aq, kp, mo, cM, lx, W, bP, asI, dB, Qu, hL, B, cvE, dA, oe, nx, aA, j, q, er, qH, eA, azn, nv, aT, ae, jf, s, Ar, nO, oV, Y, ev, iU, r, en, cl, t, nC, ck, cT, kP, c, Dy, ac, np, bt, rA, VB, LS, Mb, jN, hF, iJ, ad, hX, Jm, iC, hd, py, Sr, arV, io, wm, fb, d, lR, ue, cW, ff, xs, eH, eZ, aF, iR, bz, cvF, es, bF, nb, bE, X, BA); #c1(PROM2) c2(NP_001159449) c3(prominin-2 precursor) c4(64099, 64100) c5(64101, 64102, 64103, 64104, 64105, 64106, 64107, 64108, 64109, 64110, 64111, 64112, 64113, 64114, 64115, 64116, 64117, 64118, 64119, 64120, 64121, 64122, 64123, 64124, 64125, 64126, 64127, 64128) c6(64129, 64130, 64131, 64132, 64133, 64134, 64135, 64136) c7(Uc, ae, fr, aR, ad); #c1(PROP1) c2(NP_006252) c3(homeobox protein prophet of Pit-1) c4(163300) c5(163301, 163302, 163303, 163304, 163305, 163306) c6( ) c7(yo, bBJ, s, cvH, Dr, aX, acz, Rj, auC, hR, v, UE, bBS, ml, UF, OH, xb, bHF, yn, biN); #c1(PROS1) c2(NP_000304) c3(vitamin K-dependent protein S preproprotein) c4(6757) c5(6758, 6759, 6760, 6761, 6762, 6763, 6764, 6765) c6( ) c7(P, gh, sY, T, dD, dA, fR, WA, ea, rQ, e, ha, wo, vr, n, Fb, j, ci, eq, eW, ad, bqs, afm, cdJ, cvJ, aT, ey, ji, ae, Ub, aW, sq, cr, fQ, YG, g, t, uC, cvl, hq, Cq, pu, sn, GU, Jr, ac, ag, cvw, wx, m, LS); #c1(PROSER1) c2(NP_079414) c3(proline and serine-rich protein 1) c4(218035) c5(218036, 218037, 218038, 218039, 218040, 218041) c6( ) c7(IV); #c1(PROSER1) c2(XP_005266602) c3(proline and serine-rich protein 1 isoform X1) c4(280246) c5(280247, 280248, 280249, 280250, 280251, 280252) c6( ) c7(IV); #c1(PROX1) c2(XP_011508075) c3(prospero homeobox protein 1 isoform X2) c4(298537, 298538) c5( ) c6( ) c7(bL, T, aC, dA, ht, al, it, bB, n, v, ci, pz, q, eA, BI, jz, aE, acA, aoh, aT, Vv, K, hu, De, qw, s, aW, ff, r, aq, yq, en, cm, gk, ac, bE, Vy, ym, bD, cB, B); #c1(PROX1) c2(XP_011508076) c3(prospero homeobox protein 1 isoform X1) c4(298539, 298540) c5(298541, 298542, 298543, 298544, 298545, 298546, 298547, 298548, 298549, 298550, 298551, 298552, 298553, 298554, 298555, 298556, 298557, 298558, 298559, 298560, 298561, 298562, 298563, 298564, 298565, 298566, 298567, 298568, 298569, 298570, 298571, 298572, 298573) c6( ) c7(bL, T, aC, dA, ht, al, it, bB, n, v, ci, pz, q, eA, Bl, jz, aE, acA, aoh, aT, Vv, K, hu, de, qw, s, aW, ff, r, aq, yq, en, cm, gk, ac, bE, Vy, ym, bD, cB, B); #c1(PROZ) c2(NP_001243063) c3(vitamin K-dependent protein Z isoform 1 precursor) c4( ) c5(89701, 89702, 89703, 89704, 89705, 89706, 89707) c6( ) c7(jv, sn, ac, uC, eR, ag, e, aw, cy, cvK, dy, uF, hH, aaD, eL, eu, ek); #c1(PROZ) c2(NP_003882) c3(vitamin K-dependent protein Z isoform 2 precursor) c4(141791) c5(141792, 141793, 141794, 141795, 141796, 141797, 141798, 141799) c6( ) c7(jv, sn, ac, uC, eR, ag, e, aw, cy, cvK, dy, uF, hH, aaD, eL, eu, ek); #c1(PROZ) c2(XP_011535827) c3(vitamin K-dependent protein Z isoform X1) c4(333038) c5(333039) c6( ) c7(jv, sn, ac, uC, eR, ag, e, aw, cy, cvK, dy, uF, hH, aaD, eL, eu, eK); #c1(PRPF19) c2(NP_055317) c3(pre-mRNA-processing factor 19) c4(180409) c5(180410, 180411, 180412, 180413, 180414, 180415) c6( ) c7(bM, wr, aW, aA, aT, K); #c1(PRPF3) c2(XP_011508434) c3(U4/U6 small nuclear ribonucleoprotein Prp3 isoform X5) c4(299144) c5(299145, 299146, 299147, 299148, 299149) c6( ) c7(xX, lR, cvL, aW, aT, nw, nC); #c1(PRPF31) c2(XP_006726118) c3(U4/U6 small nuclear ribonucleoprotein Prp31 isoform X1) c4(297079, 297080, 297081, 297082, 297083, 297084) c5(297085, 297086, 297087, 297088, 297089, 297090, 297091, 297092, 297093, 297094, 297095, 297096, 297097, 297098, 297099, 297100, 297101, 297102, 297103, 297104, 297105, 297106, 297107, 297108, 297109, 297110, 297111, 297112, 297113, 297114, 297115, 297116, 297117, 297118, 297119, 297120) c6( ) c7(xX, lR, nb, W, alC, aW, ff, n, nw, nC, en, QK, cvM, dr, de); #c1(PRPF38B) c2(XP_011539987) c3(pre-mRNA-splicing factor 38B isoform X2) c4( ) c5(338306) c6( ) c7(T, dB, aW, JI, awa, AH, awb, aT, aA, aF, ci, K, g, B); #c1(PRPF38B) c2(XP_011539990) c3 (pre-mRNA-splicing factor 388 isoform X4) c4( ) c5(338307, 338308, 338309, 338310, 338311, 338312) c6( ) c7(T, dB, aW, JI, awa, AH, awb, aT, aA, aF, ci, K, g, B); #c1(PRPF4) c2(NP_001231855) c3(U4/U6 small nuclear ribonucleoprotein Prp4 isoform 2) c4(87571) c5(87572, 87573, 87574, 87575, 87576, 87577) c6( ) c7(T, xX, m, t, lB, B); #c1(PRPF4) c2(NP_004688) c3(U4/U6 small nuclear ribonucleoprotein Prp4 isoform 1) c4(148844) c5(148845, 148846, 148847, 148848, 148849, 148850) c6( ) c7(T, xX, m, t, lB, B); #c1(PRPF4DA) c2(NP_060362) c3(pre-mRNA-processing factor 40 homolog A) c4(195579, 195580) c5(195581, 195582, 195583, 195584, 195585, 195586, 195587, 195588, 195589, 195590, 195591) c6( ) c7(li, rR, bE, bB, bc, aR); #c1(PRPF40A) c2(XP_005246733) c3(pre-mRNA-processing factor 40 homolog A isoform X1) c4(267397) c5(267398) c6( ) c7(Qi, rR, bE, bB, bc, aR); #c1(PRPF40A) c2(XP_005246734) c3(pre-mRNA-processing factor 40 homolog A isoform X4) c4(267399) c5(267400, 267401, 267402, 267403, 267404, 267405) c6( ) c7(Qi, rR, bE, bB, bc, aR); #c1(PRPF40A) c2(XP_005246735) c3(pre-mRNA-processing factor 40 homolog A isoform X6) c4(267406) c5(267407, 267408, 267409, 267410, 267411, 267412) c6( ) c7(Qi, rR, bE, bB, bc, aR); #c1(PRPF40A) c2(XP_011509748) c3(pre-mRNA-processing factor 40 homolog A isoform X2) c4( ) c5(300306, 300307, 300308, 300309, 300310, 300311) c6( ) c7(Qi, rR, bE, bB, bc, aR); #c1(PRPF4B) c2(NP_003904) c3(serine/threonine-protein kinase PRP4 homolog) c4(142025, 142026) c5(142027, 142028, 142029, 142030, 142031, 142032, 142033, 142034, 142035, 142036, 142037, 142038, 142039, 142040, 142041, 142042) c6( ) c7(T, aT, aA, aF, bWB, ci, m, t, B); #c1(PRPF6) c2(NP_036601) c3(pre-mRNA-processing factor 6) c4(174723, 174724) c5(174725, 174726, 174727, 174728, 174729, 174730, 174731, 174732, 174733, 174734, 174735, 174736) c6( ) c7(T, jh, bB, mS, bgR, cvN, cc, mW, oj, bK, K, mY, mV, awL, ml, xX, axu, xs, nC, kC, mZ, dr, mX, Qi, akQ, bE, mx, rx, XO, bgW, mT); #c1(PRPF8) c2(NP_006436) c3(pre-mRNA-processing-splicing factor 8) c4(165211, 165212) c5(165213, 165214, 165215, 165216, 165217, 165218, 165219, 165220, 165221, 165222, 165223, 165224) c6( ) c7(xX, lR, h, aVK, nC, ad, cvO); #c1(PRPH) c2(NP_006253) c3(peripherin) c4(163307) c5(163308, 163309, 163310, 163311, 163312, 163313) c6( ) c7(bGg, bjZ, nx, Vd, fu, PJ, Sr, K, aqX, bU, db, xX, lR, nk, cm, nC, amf, W, nb, c, cvP, Be, nw, OL); #c1(PRPH2) c2(NP_000313) c3(peripherin-2) c4(6963) c5(6964, 6965, 6966, 6967, 6968, 6969) c6( ) c7(bGg, bjZ, nx, cvQ, aeu, cap, Nh, Sr, aqX, db, cvR, xX, lR, cvS, nk, nC, Uk, amf, W, nb, bnP, cvP, uJ, nw, OL, GF, de); #c1(PRPS1) c2(NP_002755) c3(ribose-phosphate pyrophosphokinase 1 isoform 1) c4(131710, 131711, 131712, 131713) c5(131714, 131715, 131716, 131717, 131718, 131719, 131720, 131721, 131722, 131723, 131724, 131725, 131726, 131727, 131728, 131729, 131730, 131731, 131732, 131733, 131734, 131735, 131736, 131737, 131738, 131739, 131740, 131741) c6( ) c7(x, axa, cZ, T, d, bU, cvV, cvW, cvT, aqJ, mG, FV, bhi, bCo, vd, k, bX, dd, as, cvU, Bh); #c1(PRPS1L1) c2(NP_787082) c3(ribose-phosphate pyrophosphokinase 3) c4(252090) c5(252091, 252092, 252093, 252094, 252095, 252096) c6( ) c7(bL, cZ, bU, bCo, mG, FV, bhi, EQ, vd, bX, dd, Bh, cd); #c1(PRPS2) c2(NP_001034180) c3(ribose-phosphate pyrophosphokinase 2 isoform 1) c4(31574) c5(31575, 31576, 31577, 31578, 31579, 31580) c6( ) c7(T); #c1(PRPS2) c2(NP_002756) c3(ribose-phosphate pyrophosphokinase 2 isoform 2) c4(131742) c5(131743, 131744, 131745, 131746, 131747, 131748) c6( ) c7(T); #c1(PRPSAP1) c2(NP_002757) c3(phosphoribosyl pyrophosphate synthase-associated protein 1) c4(131749, 131750) c5(131751, 131752, 131753, 131754, 131755, 131756, 131757) c6( ) c7(nH); #c1(PRPSAP1) c2(XP_011523336) c3(phosphoribosyl pyrophosphate synthase-associated protein 1 isoform X1) c4( ) c5(317872, 317873, 317874, 317875, 317876) c6( ) c7(nH); #c1(PRPSAP2) c2(NP_001230865) c3 (phosphoribosyl pyrophosphate synthase-associated protein 2 isoform 2) c4(86867, 86868) c5(86869, 86870, 86871, 86872, 86873, 86874, 86875, 86876, 86877, 86878, 86879) c6( ) c7(nH); #c1(PRPSAP2) c2(NP_001230869) c3(phosphoribosyl pyrophosphate synthase-associated protein 2 isoform 3) c4(86880) c5(86881, 86882, 86883, 86884, 86885, 86886, 86887, 86888, 86889, 86890, 86891, 86892, 86893, 86894, 86895, 86896, 86897, 86898, 86899, 86900) c6( ) c7(nH); #c1(PRPSAP2) c2(XP_005256782) c3(phosphoribosyl pyrophosphate synthase-associated protein 2 isoform X1) c4(274487, 274488, 274489, 274490, 274491, 274492, 274493) c5(274494, 274495, 274496, 274497, 274498, 274499, 274500, 274501, 274502, 274503, 274504, 274505) c6( ) c7(nH); #c1(PRPSAP2) c2(XP_005256786) c3(phosphoribosyl pyrophosphate synthase-associated protein 2 isoform X2) c4( ) c5(274506, 274507, 274508, 274509, 274510) c6( ) c7(nH); #c1(PRPSAP2) c2(XP_011522262) c3(phosphoribosyl pyrophosphate synthase-associated protein 2 isoform X3) c4(316914) c5(316915, 316916, 316917, 316918, 316919, 316920, 316921, 316922, 316923, 316924, 316925, 316926, 316927, 316928, 316929, 316930, 316931, 316932, 316933, 316934, 316935, 316936, 316937, 316938, 316939, 316940, 316941, 316942, 316943, 316944) c6( ) c7(nH); #c1(PRR11) c2(NP_060774) c3(proline-rich protein (1) c4(197719) c5(197720, 197721, 197722, 197723, 197724, 197725) c6( ) c7(bz); #c1(PRR13) c2(NP_001005354) c3(proline-rich protein 13 isoform 2) c4(21446, 21447) c5( ) c6( ) c7(eM, iu, g); #c1(PRR13) c2(NP_060927) c3(proline-rich protein 13 isoform 1) c4(198728) c5(198729, 198730, 198731, 198732, 198733, 198734, 198735, 198736, 198737, 198738, 198739, 198740, 198741, 198742, 198743, 198744, 198745, 198746, 198747, 198748) c6( ) c7(eM, iu, g); #c1(PRR15) c2(XP_011513501) c3(proline-rich protein 15 isoform X1) c4(305789, 305790) c5(305791, 305792, 305793, 305794, 305795, 305796, 305797, 305798, 305799, 305800, 305801, 305802) c6( ) c7(aw, gX, r, gZ, q, [e); #c1(PRR16) c2(NP_001287712) c3(protein Largen isoform 1) c4(112613, 112614) c5(112615, 112616, 112617, 112618, 112619, 112620, 112621, 112622) c6( ) c7(fq); #c1(PRR16) c2(NP_057728) c3(protein Largen isoform 2) c4(192541, 192542) c5(192543, 192544, 192545, 192546, 192547, 192548) c6( ) c7(fq); #c1(PRR16) c2(XP_011541755) c3(protein Largen isoform X1) c4( ) c5(340353) c6( ) c7(fq); #c1(PRR3) c2(NP_001070965) c3(proline-rich protein 3 isoform b) c4(35093) c5(35094, 35095, 35096, 35097, 35098, 35099, 35100, 35101, 35102, 35103, 35104, 35105, 35106, 35107, 35108, 35109, 35110, 35111, 35112, 35113, 35114, 35115, 35116, 35117, 35118) c6( ) c7(hD, mR, eZ, mP); #c1(PRR3) c2(NP_079539) c3(proline-rich protein 3 isoform a) c4(219109, 219110) c5(219111, 219112, 219113, 219114, 219115, 219116, 219117, 219118, 219119, 219120, 219121, 219122, 219123, 219124, 219125, 219126, 219127, 219128, 219129, 219130, 219131, 219132, 219133, 219134, 219135, 219136, 219137, 219138, 219139, 219140, 219141, 219142, 219143, 219144, 219145, 219146, 219147, 219148, 219149, 219150, 219151, 219152, 219153, 219154, 219155, 219156, 219157, 219158, 219159, 219160) c6( ) c7(hD, mR, eZ, mP); #c1(PRR34) c2(NP_060750) c3(proline-rich protein 34) c4(197589) c5(197590, 197591, 197592, 197593, 197594, 197595, 197596, 197597, 197598, 197599, 197600, 197601) c6( ) c7(m); #c1(PRR5) c2(NP_001017528) c3(proline-rich protein 5 isoform 2) c4(26564, 26565, 26566) c5(26567, 26568, 26569, 26570, 26571, 26572, 26573, 26574, 26575, 26576, 26577, 26578, 26579, 26580, 26581, 26582, 26583) c6(26584, 26585) c7(aW, aw, q, aT); #c1(PRR5) c2(NP_001185650) c3(proline-rich protein 5 isoform 4) c4(76030) c5(76031, 76032, 76033, 76034, 76035, 76036) c6( ) c7(aW, aw, q, aT); #c1(PRR5) c2(NP_851850) c3(proline-rich protein 5 isoform 1) c4(254680) c5(254681, 254682, 254683, 254684, 254685, 254686, 254687, 254688, 254689) c6( ) c7(aW, aw, q, aT); #c1(PRR9) c2(XP_011508108) c3(proline-rich protein 9 isoform X2) c4(298589) c5(298590) c6( ) c7(cr); #c1(PRR9) c2(XP_011508109) c3(proline-rich protein 9 isoform X1) c4( ) c5(298591, 298592, 298593, 298594, 298595) c6( ) c7(cr); #c1(PRRC1) c2(NP_001273737) c3(protein PRRC1 isoform 1) c4(105731) c5(105732, 105733, 105734, 105735, 105736, 105737, 105738, 105739, 105740, 105741, 105742, 105743) c6( ) c7(x, ey, j, k, fe); #c1(PRRC1) c2(NP_570721) c3(protein PRRC1 isoform 2) c4(234237, 234238, 234239, 234240, 234241) c5(234242, 234243, 234244, 234245, 234246, 234247, 234248, 234249, 234250, 234251, 234252, 234253, 234254, 234255, 234256, 234257, 234258, 234259, 234260, 234261, 234262, 234263, 234264, 234265, 234266, 234267, 234268, 234269, 234270, 234271, 234272, 234273) c6( ) c7(x, ey, j, k, fe); #c1(PRRC2A) c2(NP_542417) c3(protein PRRC2A) c4(232796, 232797, 232798, 232799, 232800) c5(232801, 232802, 232803, 232804, 232805, 232806, 232807, 232808, 232809, 232810, 232811, 232812, 232813, 232814, 232815, 232816, 232817, 232818, 232819, 232820, 232821, 232822, 232823, 232824, 232825, 232826, 232827, 232828, 232829) c6( ) c7(y, A, iQ, ac, od, z, iW, e, aw, ff, dV, eZ, h, v, j, en, iV, bD, lf, aue, eu); #c1(PRRC2A) c2(XP_011513192) c3(protein PRRC2A isoform X9) c4(305210, 305211) c5(305212, 305213, 305214, 305215, 305216, 305217, 305218, 305219, 305220, 305221, 305222, 305223, 305224, 305225, 305226, 305227, 305228, 305229, 305230) c6( ) c7(y, A, iQ, ac, od, z, iW, e, aw, ff, dV, eZ, h, v, j, en, iV, bD, lf, que, eu); #c1(PRRC2A) c2(XP_011545591) c3(protein PRRC2A isoform XI) c4( ) c5(344364, 344365, 344366, 344367, 344368, 344369, 344370, 344371, 344372, 344373, 344374, 344375, 344376, 344377, 344378, 344379, 344380, 344381, 344382, 344383, 344384, 344385, 344386, 344387, 344388, 344389, 344390, 344391, 344392, 344393, 344394, 344395, 344396, 344397, 344398, 344399, 344400, 344401, 344402, 344403, 344404, 344405, 344406, 344407, 344408, 344409, 344410, 344411, 344412, 344413, 344414, 344415, 344416, 344417, 344418, 344419, 344420, 344421, 344422, 344423, 344424, 344425) c6( ) c7(y, A, id, ac, od, z, iW, e, aw, ff, dV, eZ, h, v, j, en, iV, bD, lf, que, eu); #c1(PRRC2A) c2(XP_011545592) c3(protein PRRC2A isoform X9) c4(344426, 344427, 344428) c5( ) c6( ) c7(y, A, iQ, ac, od, z, iW, e, aw, ff, dV, eZ, h, v, j, en, iV, bD, lf, aue, eu); #c1(PRRC2A) c2(XP_011545992) c3(protein PRRC2A isoform X2) c4(344844, 344845, 344846, 344847) c5( ) c6( ) c7(y, A, iQ, ac, od, z, iW, e, aw, ff, dV, eZ, h, v, j, en, iV, bD, lf, aue, eu); #c1(PRRC2A) c2(XP_011545994) c3 (protein PRRC2A isoform X9) c4(344848) c5( ) c6( ) c7(y, A, iQ, ac, od, z, iW, e, aw, ff, dV, eZ, h, v, j, en, iV, bD, lf, aue, eu); #c1(PRRC2A) c2(XP_011546211) c3(protein PRRC2A isoform X4) c4(344902, 344903, 344904) c5( ) c6( ) c7(y, A, iQ, ac, od, z, iW, e, aw, ff, dV, eZ, h, v, j, en, iV, bD, lf, que, eu); #c1(PRRC2A) c2(XP_011546212) c3 (protein PRRC2A isoform X9) c4(344905, 344906) c5( ) c6( ) c7(y, A, iQ, ac, od, z, iW, e, aw, ff, dV, eZ, h, v, j, en, iV, bD, lf, aue, eu); #c1(PRRC2A) c2(XP_011546213) c3(protein PRRC2A isoform X5) c4( ) c5(344907, 344908, 344909, 344910, 344911, 344912, 344913, 344914, 344915, 344916, 344917, 344918, 344919, 344920, 344921, 344922, 344923, 344924, 344925, 344926, 344927, 344928, 344929, 344930, 344931, 344932, 344933, 344934, 344935, 344936) c6( ) c7(y, A, iQ, ac, od, z, iW, e, aw, ff, dV, eZ, h, v, j, en, iV, bD, lf, aue, eu); #c1(PRRC2A) c2(XP_011546382) c3(protein PRRC2A isoform X6) c4( ) c5(345114, 345115, 345116, 345117, 345118, 345119, 345120, 345121, 345122, 345123, 345124, 345125, 345126, 345127, 345128, 345129, 345130, 345131, 345132, 345133, 345134, 345135, 345136, 345137, 345138, 345139, 345140, 345141, 345142, 345143, 345144, 345145, 345146, 345147, 345148, 345149, 345150, 345151, 345152, 345153, 345154, 345155, 345156, 345157, 345158, 345159, 345160, 345161, 345162, 345163, 345164, 345165, 345166, 345167) c6( ) c7(y, A, iQ, ac, od, z, iW, e, aw, ff, dV, eZ, h, v, j, en, iV, bD, lf, aue, eu); #c1(PRRC2C) c2(NP_055987) c3(protein PRRC2C) c4(184920, 184921) c5(184922, 184923, 184924, 184925, 184926, 184927, 184928, 184929, 184930, 184931, 184932, 184933, 184934, 184935, 184936, 184937, 184938, 184939, 184940, 184941, 184942, 184943, 184944, 184945, 184946, 184947, 184948, 184949, 184950, 184951) c6( ) c7(av); #c1(PRRC2C) c2(XP_005245072) c3(protein PRRC2C isoform X1) c4( ) c5(266258, 266259, 266260, 266261, 266262) c6( ) c7(av); #c1(PRRC2C) c2(XP_005245073) c3(protein PRRC2C isoform X2) c4(266263) c5( ) c6( ) c7(av); #c1(PRRC2C) c2(XP_005245074) c3(protein PRRC2C isoform X3) c4(266264) c5( ) c6( ) c7(av); #c1(PRRC2C) c2(XP_005245076) c3(protein PRRC2C isoform X6) c4(266265) c5( ) c6( ) c7(av); #c1(PRRC2C) c2(XP_005245080) c3(protein PRRC2C isoform X9) c4( ) c5(266266) c6( ) c7(av); #c1(PRRC2C) c2(XP_005245087) c3(protein PRRC2C isoform X16) c4( ) c5(266267, 266268, 266269, 266270, 266271, 266272, 266273, 266274) c6( ) c7(av); #c1(PRRC2C) c2(XP_006711301) c3(protein PRRC2C isoform X5) c4(285868, 285869) c5( ) c6( ) c7(av); #c1(PRRG4) c2(NP_076986) c3(transmembrane gamma-carboxyglutamic acid protein 4 precursor) c4(214628) c5(214629, 214630, 214631, 214632, 214633, 214634) c6( ) c7(an); #c1(PRRT1) c2(NP_085154) c3(proline-rich transmembrane protein 1) c4(219497) c5(219498, 219499, 219500, 219501, 219502, 219503, 219504, 219505, 219506, 219507, 219508, 219509, 219510, 219511, 219512, 219513, 219514, 219515, 219516, 219517, 219518, 219519, 219520, 219521, 219522, 219523, 219524) c6( ) c7(y, A, eZ); #c1(PRRT1) c2(XP_011544625) c3(proline-rich transmembrane protein 1 isoform X1) c4(343669, 343670) c5(343671, 343672, 343673, 343674, 343675, 343676, 343677, 343678, 343679, 343680, 343681, 343682, 343683, 343684, 343685, 343686, 343687, 343688, 343689, 343690, 343691, 343692, 343693, 343694, 343695, 343696, 343697, 343698, 343699, 343700, 343701, 343702, 343703, 343704, 343705, 343706, 343707, 343708, 343709, 343710, 343711, 343712, 343713, 343714, 343715, 343716, 343717, 343718, 343719, 343720, 343721, 343722, 343723, 343724, 343725, 343726, 343727, 343728, 343729, 343730, 343731, 343732, 343733, 343734, 343735, 343736, 343737, 343738, 343739, 343740, 343741, 343742, 343743, 343744, 343745, 343746, 343747, 343748, 343749, 343750, 343751, 343752) c6( ) c7(y, A, eZ); #c1(PRRT1) c2(XP_011545623) c3(proline-rich transmembrane protein 1 isoform X3) c4( ) c5(344552) c6( ) c7(y, A, eZ); #c1(PRRT2) c2(NP_001243372) c3(proline-rich transmembrane protein 2 isoform 3) c4(90585) c5(90586, 90587, 90588, 90589, 90590, 90591) c6( ) c7(gh, T, azf, agU, fd, bB, ur, lR, aR, aE, aEX, dV, aU, K, pk, De, aW, axu, cm, akf, iP, eM, nB, cvY, vy, cj, m, bVk, nA, eh, cd, ai, cc, ayQ, h, dx, ayn, Ni, aq, vo, cuY, B, bL, fG, aA, bX, FR, V, q, eA, hb, Nw, aT, Wk, bsE, akr, rz, ae, Y, r, en, t, xw, iK, pb, akq, ck, ll, bt, iW, ha, dG, uu, Fb, ad, dP, hd, LA, fb, cuX, d, qt, yx, ff, aF, EZ, bz, DF, dR, cvX, xZ, bE, akm, uR, y, X); #c1(PRRT2) c2(NP_660282) c3 (proline-rich transmembrane protein 2 isoform 1) c4(240627) c5(240628, 240629, 240630, 240631, 240632, 240633) c6( ) c7(gh, T, azf, agU, fd, bB, ur, lR, aR, aE, aEX, dV, aU, K, pk, De, aW, axu, cm, akf, iP, eM, nB, cvY, vy, cj, m, bVk, nA, eh, cd, ai, cc, ayQ, h, dx, ayn, Ni, aq, vo, cuY, B, bL, fG, aA, bX, FR, V, q, eA, hb, Nw, aT, Wk, bsE, akr, rz, ae, Y, r, en, t, xw, ik, pb, akq, ck, ll, bt, iW, ha, dG, uu, Fb, ad, dP, hd, LA, fb, cuX, d, qt, yx, ff, aF, EZ, bz, DF, dR, cvX, xZ, bE, akm, uR, y, X); #c1(PRRX1) c2(NP_008833) c3(paired mesoderm homeobox protein 1 isoform pmx-1a) c4(168925) c5(168926, 168927, 168928, 168929, 168930) c6( ) c7(T, wL, al, n, v, chG, q, ad, aT, K, cwa, af, iU, aW, r, cvZ, cnr, f, bz, ck, eM, iT, cnw, bt); #c1(PRRX1) c2(NP_073207) c3(paired mesoderm homeobox protein 1 isoform pmx-1b) c4(212836) c5(212837, 212838, 212839, 212840, 212841, 212842, 212843, 212844) c6( ) c7(T, wL, al, n, v, chG, q, ad, aT, K, cwa, af, iU, aW, r, cvZ, cnr, f, bz, ck, eM, iT, cnw, bt); #c1(PRRX2) c2(NP_057391) c3(paired mesoderm homeobox protein 2) c4(190531) c5(190532, 190533, 190534, 190535, 190536, 190537, 190538) c6( ) c7(T, eM, Vy, ayB, cvZ, K, ym, PJ, f, ae, af, ad); #c1(PRSS1) c2(NP_002760) c3(trypsin-1 preproprotein) c4( ) c5(131764, 131765, 131766, 131767, 131768, 131769, 131770, 131771, 131772, 131773, 131774, 131775) c6( ) c7(Mb, T, aC, cwb, jx, bB, aj, qj, ci, iG, aMd, alx, ad, J, eJ, aE, oz, nX, aMj, wb, K, aCO, aKG, dQ, dF, A, Y, kp, aF, g, HI, dr, OC, bF, ar, bE, cwc, afd, aMc, m, lS, X, ao, ayZ, pC); #c1(PRSS1) c2(XP_011514713) c3(trypsin-1 isoform X1) c4(307365, 307366, 307367, 307368) c5(307369, 307370, 307371, 307372, 307373, 307374, 307375, 307376, 307377, 307378, 307379, 307380, 307381, 307382, 307383, 307384) c6( ) c7(Mb, T, aC, cwb, jx, bB, aj, qj, ci, iG, aMd, alx, ad, J, eJ, aE, oz, nX, aMj, wb, K, aCO, aKG, dQ, dF, A, Y, kp, aF, g, HI, dr, OC, bF, ar, bE, cwc, afd, aMc, m, lS, X, ao, ayZ, pC); #c1(PRSS12) c2(NP_003610) c3(neurotrypsin precursor) c4(139577) c5(139578, 139579, 139580, 139581, 139582, 139583, 139584) c6( ) c7(ac, nA, cwd); #c1(PRSS16) c2(NP_005856) c3(thymus-specific serine protease) c4(159946, 159947, 159948, 159949) c5(159950, 159951, 159952, 159953, 159954, 159955, 159956, 159957, 159958, 159959, 159960, 159961, 159962, 159963, 159964, 159965, 159966, 159967, 159968, 159969, 159970, 159971, 159972, 159973) c6( ) c7(hM, pd, A); #c1(PRSS2) c2(NP_002761) c3(trypsin-2 isoform 2 preproprotein) c4(131776) c5(131777, 131778, 131779, 131780, 131781, 131782) c6( ) c7(T, aC, eX, iG, ci, ad, jz, eV, wb, EU, ff, r, en, g, ar, zr, ru, zL, m, y, it, X, ayZ); #c1(PRSS21) c2(NP_001257381) c3(testisin isoform 4 preproprotein) c4(95290) c5(95291, 95292, 95293, 95294, 95295) c6( ) c7(bL, dA, sO, lW, h, rO, mf, GR); #c1(PRSS21) c2(NP_006790) c3(testisin isoform 1 preproprotein) c4(168091) c5(168092, 168093, 168094, 168095, 168096, 168097, 168098, 168099) c6( ) c7(bL, dA, sD, lW, h, rO, mf, GR); #c1(PRSS21) c2(NP_659205) c3(testisin isoform 2 preproprotein) c4(239741) c5(239742, 239743, 239744, 239745, 239746, 239747, 239748) c6( ) c7(bL, dA, sO, lW, h, rO, mf, GR); #c1(PRSS21) c2(NP_659206) c3(testisin isoform 3 preproprotein) c4(239749) c5(239750, 239751, 239752, 239753, 239754, 239755) c6( ) c7(bL, dA, sO, lW, h, rO, mf, GR); #c1(PRSS22) c2(NP_071402) c3(brain-specific serine protease 4 precursor) c4(211443) c5(211444, 211445, 211446, 211447, 211448, 211449, 211450, 211451) c6( ) c7(T, bE, bB, aq, m, eA, t, B); #c1(PRSS23) c2(NP_001280107) c3(serine protease 23 isoform b) c4(111296) c5(111297, 111298, 111299, 111300, 111301, 111302, 111303, 111304) c6(111305, 111306) c7(aW, T, v, Y, aT); #c1(PRSS23) c2(NP_001280109) c3(serine protease 23 isoform c precursor) c4( ) c5(111307, 111308, 111309, 111310, 111311, 111312, 111313, 111314, 111315, 111316, 111317, 111318, 111319, 111320) c6(111321, 111322, 111323, 111324) c7(aW, T, v, Y, aT); #c1(PRSS23) c2(NP_009104) c3(serine protease 23 isoform a) c4(170677, 170678) c5(170679, 170680) c6( ) c7(aW, T, v, Y, aT); #c1(PRSS27) c2(NP_114154) c3(serine protease 27 precursor) c4(222390, 222391) c5(222392, 222393, 222394, 222395, 222396, 222397, 222398, 222399, 222400, 222401, 222402, 222403, 222404, 222405, 222406, 222407) c6(222408, 222409, 222410, 222411) c7(ck, pq, aiu, gM, T, oT, pc, iU, ag, bs, kD, n, pg, nC, aqf, pm, bw, bt, br, yR); #c1(PRSS3) c2(NP_001184026) c3(trypsin-3 isoform 3 preproprotein) c4(75257, 75258) c5(75259, 75260, 75261, 75262, 75263, 75264, 75265, 75266, 75267, 75268, 75269, 75270) c6( ) c7(EB, T, aE, eM, JI, aW, eL, le, eX, aT, it, eV, dL, bk, wb, iG, ra, V, ae, ad, vi); #c1(PRSS3) c2(NP_001184027) c3(trypsin-3 isoform 4 preproprotein) c4(75271) c5(75272, 75273, 75274, 75275, 75276, 75277) c6( ) c7(EB, T, aE, eM, JI, aW, eL, le, eX, aT, it, eV, dL, bK, wb, iG, ra, V, ae, ad, vi); #c1(PRSS3) c2(NP_002762) c3(trypsin-3 isoform 2 preproprotein) c4(131783) c5(131784, 131785, 131786, 131787, 131788, 131789) c6( ) c7(EB, T, aE, eM, JI, aW, eL, le, eX, aT, it, eV, dL, bK, wb, iG, ra, V, ae, ad, vi); #c1(PRSS3) c2(NP_031369) c3(trypsin-3 isoform 1 preproprotein) c4(171973) c5(171974, 171975, 171976, 171977, 171978, 171979) c6( ) c7(EB, T, aE, eM, JI, aW, eL, le, eX, aT, it, eV, dL, bK, wb, iG, ra, V, ae, ad, vi); #c1(PRSS33) c2(NP_690851) c3(serine protease 33 precursor) c4(244975) c5(244976, 244977, 244978, 244979, 244980, 244981) c6( ) c7(Qi, bEg); #c1(PRSS35) c2(NP_001163894) c3(inactive serine protease 35 precursor) c4(66489) c5(66490, 66491, 66492, 66493, 66494, 66495, 66496, 66497, 66498, 66499, 66500, 66501, 66502, 66503) c6(66504, 66505, 66506, 66507) c7(Na, amJ, Nc, cz); #c1(PRSS50) c2(NP_037402) c3(probable threonine protease PRSS50 precursor) c4(175309, 175310) c5(175311, 175312, 175313, 175314, 175315, 175316, 175317, 175318, 175319, 175320, 175321, 175322, 175323, 175324, 175325, 175326) c6( ) c7(aW, Lb, np, q, aT); #c1(PRSS53) c2(NP_001034592) c3(serine protease 53 precursor) c4(31889) c5(31890, 31891, 31892, 31893, 31894, 31895, 31896, 31897) c6(31898, 31899) c7(an); #c1(PRSS55) c2(NP_001183949) c3(serine protease 55 isoform 2 precursor) c4(75239) c5(75240, 75241, 75242, 75243, 75244, 75245) c6( ) c7(T, Tb, DD, aZ, if, e, aA, bB, fd, v, lR, ci, qN, fr, cd, ad, ai, eA, DJ, Dq, aT, cy, ae, cp, jf, asp, s, nO, aW, Y, oV, ff, aF, en, cl, dw, aal, t, bz, pb, cm, DT, eM, iA, bE, hL, agf, mf, m, y, iu); #c1(PRSS55) c2(NP_940866) c3(serine protease 55 isoform 1 precursor) c4(259984) c5(259985, 259986, 259987, 259988, 259989, 259990) c6( ) c7(T, Tb, DD, aZ, if, e, aA, bB, fd, v, lR, ci, qN, fr, cd, ad, ai, eA, DJ, Dq, aT, cy, ae, cp, jf, asp, s, nO, aW, Y, oV, ff, aF, en, cl, dw, aal, t, bz, pb, cm, DT, eM, iA, bE, hL, agf, mf, m, y, iu); #c1(PRSS57) c2(NP_999875) c3(serine protease 57 precursor) c4(266092) c5(266093, 266094, 266095, 266096, 266097, 266098, 266099, 266100) c6(266101, 266102) c7(BD, cwe, NL, bed, pR, la, Ph, bOu); #c1(PRSS58) c2(NP_001001317) c3(serine protease 58 precursor) c4(18152) c5(18153, 18154, 18155, 18156, 18157, 18158, 18159, 18160, 18161, 18162, 18163, 18164, 18165, 18166, 18167, 18168, 18169) c6(18170, 18171, 18172, 18173) c7(bF, ar, A, aE, Y, cwc, X, afd, aMj, ao, kp, iG, aCO, aMd, alx, dr, OC); #c1(PRSS8) c2(NP_002764) c3(prostasin preproprotein) c4(131797, 131798) c5(131799, 131800, 131801, 131802, 131803, 131804, 131805, 131806, 131807, 131808, 131809, 131810) c6( ) c7(T, jv, id, Uc, aA, cz, aj, fr, ci, q, J, ad, XX, aT, h, K, lQ, ae, aW, t, xd, fk, CZ, m, ao); #c1(PRTFDC1) c2(NP_001269715) c3 (phosphoribosyltransferase domain-containing protein 1 isoform 2) c4(102683, 102684) c5(102685, 102686, 102687, 102688, 102689, 102690, 102691, 102692, 102693, 102694, 102695, 102696) c6( ) c7(t, ff, en); #c1(PRTFDC1) c2(NP_064585) c3(phosphoribosyltransferase domain-containing protein 1 isoform 1) c4(203112) c5(203113, 203114, 203115, 203116, 203117, 203118) c6( ) c7(t, ff, en); #c1(PRTFDC1) c2(XP_011517891) c3(phosphoribosyltransferase domain-containing protein 1 isoform X4) c4(311790) c5(311791, 311792, 311793, 311794, 311795, 311796) c6( ) c7(t, ff, en); #c1(PRTG) c2(NP_776175) c3(protogenin precursor) c4(250939) c5(250940, 250941, 250942, 250943, 250944, 250945) c6( ) c7(yA); #c1(PRTN3) c2(NP_002768) c3(myeloblastin precursor) c4(131848, 131849, 131850) c5(131851, 131852, 131853, 131854, 131855, 131856, 131857, 131858, 131859, 131860, 131861, 131862) c6( ) c7(T, os, pF, am, id, eR, lo, oZ, aoj, aY, er, yM, xE, DK, Ot, aT, hn, dL, ji, eL, N, hm, aa, ao, DA, anP, iU, bJV, brg, aW, eZ, iV, pl, ve, aYt, ck, zr, iT, uz, aS, EY, y, bt, fh, hv); #c1(PRUNE) c2(NP_001290158) c3(protein prune homolog isoform 2) c4(114982) c5(14983, 114984, 114985, 114986, 114987, 114988, 114989, 114990, 114991, 114992) c6( ) c7(hW, hV, aW, au, aA, aT, cm); #c1(PRUNE) c2(NP_001290171) c3(protein prune homolog isoform 3) c4(115021, 115022, 115023, 115024) c5(115025, 115026, 115027, 115028, 115029, 115030, 115031, 115032, 115033, 115034, 115035, 115036, 115037, 115038, 115039, 115040, 115041, 115042, 115043, 115044, 115045, 115046, 115047, 115048, 115049, (15050, 115051, 115052) c6( ) c7(hW, hV, aW, au, aA, aT, cm); #c1(PRUNE2) c2(NP_056040) c3(protein prune homolog 2) c4(185252) c5(185253, 185254, 185255, 185256, 185257, 185258) c6( ) c7(cZ, T, fb, hW, Y, dd, cm, ae, ad); #c1(PRUNE2) c2(XP_005251805) c3(protein prune homolog 2 isoform X8) c4(271196, 271197) c5(271198) c6( ) c7(cZ, T, fb, hW, Y, dd, cm, ae, ad); #c1(PRUNE2) c2(XP_011516625) c3(protein prune homolog 2 isoform X9) c4(310032) c5(310033, 310034, 310035, 310036, 310037, 310038) c6( ) c7(cZ, T, fb, hW, Y, dd, cm, ae, ad); #c1(PRUNE2) c2(XP_011516628) c3(protein prune homolog 2 isoform X13) c4(310039) c5( ) c6( ) c7(cZ, T, fb, hW, Y, dd, cm, ae, ad); #c1(PRUNE2) c2(XP_011516630) c3(protein prune homolog 2 isoform X16) c4( ) c5(310040, 310041, 310042, 310043, 310044, 310045) c6( ) c7(cZ, T, fb, hW, Y, dd, cm, ae, ad); #c1(PRX) c2(NP_066007) c3 (periaxin isoform 1) c4(206724) c5(206725, 206726, 206727, 206728, 206729) c6( ) c7(ql, Pt, agU, bkY, cwf, fu, bX, bU, mF, ae, ad, aa); #c1(PRX) c2(XP_011525473) c3(periaxin isoform X1) c4(319872, 319873) c5(319874, 319875, 319876, 319877, 319878, 319879, 319880, 319881, 319882, 319883, 319884, 319885, 319886, 319887) c6( ) c7(ql, Pt, agU, bkY, cwf, fu, bX, bU, mF, ae, ad, aa); #c1(PRY) c2(XP_011529813) c3 (PTPN13-like protein, Y-linked isoform X1) c4(325592, 325593) c5(325594, 325595, 325596, 325597, 325598, 325599, 325600, 325601, 325602, 325603, 325604, 325605, 325606, 325607, 325608, 325609, 325610, 325611, 325612, 325613, 325614, 325615, 325616, 325617, 325618, 325619, 325620, 325621, 325622, 325623, 325624, 325625, 325626) c6( ) c7(ae, gU, ad); #c1(PSAP) c2(NP_001035930) c3(prosaposin isoform b preproprotein) c4(33432, 33433, 33434) c5(33435, 33436, 33437, 33438, 33439, 33440, 33441, 33442, 33443, 33444, 33445, 33446) c6( ) c7(T, ww, C, cwi, Za, q, ad, AK, cwh, QU, rS, dV, aT, ey, aAt, ae, pi, ahA, afu, eG, tb, aW, Zf, bez, cwg, cr, r, BE, t, dr, ck, vU, Nk, yP, ahw, ahz, akH, wR, Lq, CZ); #c1(PSAP) c2(NP_001035931) c3(prosaposin isoform c preproprotein) c4(33447, 33448, 33449, 33450) c5(33451, 33452, 33453, 33454, 33455, 33456, 33457, 33458, 33459, 33460, 33461, 33462) c6( ) c7(T, ww, C, cwi, Za, q, ad, AK, cwh, QU, rS, dV, aT, ey, aAt, ae, pi, ahA, afu, eG, tb, aW, Zf, bez, cwg, cr, r, eE, t, dr, ck, vU, Nk, yP, ahw, ahz, akH, wR, Lq, CZ); #c1(PSAP) c2(NP_002769) c3(prosaposin isoform a preproprotein) c4(131863, 131864, 131865) c5(131866, 131867, 131868, 131869, 131870, 131871, 131872, 131873, 131874, 131875, 131876, 131877, 131878, 131879, 131880, 131881, 131882, 131883) c6( ) c7(T, ww, C, cwi, Za, q, ad, AK, cwh, QU, rS, dV, aT, ey, aAt, ae, pi, ahA, afu, eG, tb, aW, Zf, bez, cwg, cr, r, eE, t, dr, ck, vU, Nk, yP, ahw, ahz, akH, wR, Lq, CZ); #c1(PSAT1) c2(NP_066977) c3(phosphoserine aminotransferase isoform 2) c4(207944) c5(207945, 207946, 207947, 207948, 207949, 207950) c6( ) c7(gh, T, dA, WA, rQ, cz, n, Fb, ci, eW, ad, aT, ji, ae, Ub, aW, cr, lo, g, t, hq, bF, cT, pu, ac, GU, Jr, cwj, m, LS); #c1(PSAT1) c2(NP_478059) c3(phosphoserine aminotransferase isoform 1) c4(230862, 230863) c5(230864, 230865, 230866, 230867, 230868, 230869, 230870, 230871, 230872, 230873, 230874, 230875) c6( ) c7(gh, T, dA, WA, rQ, cz, n, Fb, ci, eW, ad, aT, ji, ae, Ub, aW, cr, lo, g, t, hq, bF, cT, pu, ac, GU, Jr, cwj, m, LS); #c1(PSCA) c2(NP_005663) c3(prostate stem cell antigen preproprotein) c4(158368) c5(158369, 158370, 158371, 158372, 158373, 158374, 158375, 158376, 158377, 158378) c6( ) c7(T, aC, id, ht, aA, ci, jO, eJ, ad, cwk, eW, BI, aE, Db, qp, al, io, dL, hB, nJ, ae, eL, pl, Qe, YL, ZH, CN, NE, ff, az, kt, af, en, g, fT, gX, pV, it); #c1(PSD) c2(NP_002770) c3(PH and SEC7 domain-containing protein 1 isoform 1) c4(131884, 131885) c5(131886, 131887, 131888, 131889, 131890, 131891, 131892, 131893, 131894, 131895, 131896, 131897, 131898, 131899, 131900, 131901, 131902, 131903, 131904, 131905, 131906, 131907, 131908) c6( ) c7(d, rz, bO, r, Ko, eA, iV, he, q, bN); #c1(PSD3) c2(NP_056125) c3(PH and SEC7 domain-containing protein 3 isoform a) c4(185787, 185788) c5(185789, 185790, 185791, 185792, 185793, 185794, 185795, 185796, 185797, 185798, 185799, 185800) c6( ) c7(Kc, d, ac, bE, ff, ht, bB, m, en, as, eA, t, xa, ad); #c1(PSD3) c2(NP_996792) c3(PH and SEC7 domain-containing protein 3 isoform b) c4(264547) c5(264548, 264549, 264550, 264551, 264552, 264553, 264554, 264555, 264556, 264557, 264558, 264559) c6( ) c7(Kc, d, ac, bE, ff, ht, bB, m, en, as, eA, t, xa, ad); #c1(PSD3) c2(XP_011542766) c3(PH and SEC7 domain-containing protein 3 isoform X3) c4(341390) c5( ) c6( ) c7(Kc, d, ac, bE, ff, ht, bB, m, en, as, eA, t, xa, ad); #c1(PSD4) c2(NP_036587) c3(PH and SEC7 domain-containing protein 4) c4(174638) c5(174639, 174640, 174641, 174642, 174643, 174644) c6( ) c7(xQ, T, eW, d, aW, Y, eL, aT, cQ, dL, dy, ci, q, eA, agF, cm, bz, B); #c1(PSD4) c2(XP_006712457) c3(PH and SEC7 domain-containing protein 4 isoform X5) c4( ) c5(286666) c6( ) c7(xQ, T, eW, d, aW, Y, eL, aT, cd, dL, dy, ci, q, eA, agF, cm, bz, B); #c1(PSD4) c2(XP_011509191) c3(PH and SEC7 domain-containing protein 4 isoform X6) c4( ) c5(299808) c6( ) c7(xQ, T, eW, d, aW, Y, eL, aT, cQ, dL, dy, ci, q, eA, agF, cm, bz, B); #c1(PSEN1) c2(XP_011535274) c3(presenilin-1 isoform X2) c4(332176, 332177, 332178, 332179, 332180, 332181, 332182) c5(332183, 332184, 332185, 332186, 332187, 332188, 332189, 332190, 332191, 332192, 332193, 332194, 332195, 332196, 332197, 332198, 332199, 332200, 332201, 332202, 332203, 332204, 332205, 332206, 332207, 332208, 332209, 332210, 332211, 332212, 332213, 332214, 332215, 332216, 332217, 332218, 332219, 332220, 332221, 332222, 332223, 332224) c6( ) c7(T, aaH, l, gf, Fo, ta, K, H, cwp, cws, aW, g, as, ir, cm, bb, abS, mx, U, m, R, DI, fr, ct, fK, cd, Hx, cc, ey, cwr, cwo, tx, avf, acM, hq, W, ky, dB, aXX, Lp, avF, B, L, bL, ul, acl, eR, cwq, v, eA, PW, cF, di, aTZ, WY, HC, aT, cy, aaw, an, af, ak, cwn, aaE, en, aal, f, t, wA, avz, cix, cwm, axS, tj, ac, c, cwl, wR, mf, aPT, aan, ha, VT, bS, sO, Hk, abg, ff, dq, Z, KM, dr, ek, dJ, aS, cbR, Gp, xg, CW); #c1(PSEN1) c2(XP_011535276) c3(presenilin-1 isoform X1) c4(332225, 332226, 332227, 332228, 332229, 332230, 332231, 332232, 332233) c5(332234, 332235, 332236, 332237, 332238, 332239, 332240, 332241, 332242, 332243, 332244, 332245, 332246, 332247, 332248, 332249, 332250, 332251, 332252, 332253, 332254, 332255, 332256, 332257, 332258, 332259, 332260, 332261, 332262, 332263, 332264, 332265, 332266, 332267, 332268, 332269, 332270, 332271, 332272, 332273, 332274, 332275, 332276, 332277, 332278, 332279, 332280, 332281, 332282, 332283, 332284, 332285, 332286, 332287, 332288, 332289, 332290, 332291, 332292, 332293, 332294, 332295) c6( ) c7(T, aaH, l, gf, Fo, ta, K, H, cwp, cws, aW, g, as, ir, cm, bb, abS, mx, U, m, R, Ol, fr, ct, fK, cd, Hx, cc, ey, cwr, cwo, tx, avf, acM, hq, W, ky, dB, aXX, Lp, avF, B, L, bL, ul, acL, eR, cwq, v, eA, PW, cF, di, aTZ, WY, HC, aT, cy, aaw, an, af, ak, cwn, aaE, en, aaD, f, t, wA, avz, cix, cwm, axS, tj, ac, c, cwl, wR, mf, aPT, aan, ha, VT, bS, sO, Hk, abg, ff, dq, Z, KM, dr, ek, dJ, aS, cbR, Gp, xg, CW); #c1(PSEN2) c2(NP_036618) c3(presenilin-2 isoform 2) c4(174964) c5(174965, 174966, 174967, 174968, 174969, 174970, 174971, 174972, 174973, 174974, 174975, 174976, 174977, 174978) c6( ) c7(CV, T, acL, cwt, aaH, ha, VT, aA, cz, aj, ci, il, fK, J, q, cd, ad, eA, mj, XX, di, cf, cc, WY, sO, rJ, Fo, Hk, aT, bK, K, hS, MO, H, ae, af, cwr, B, PP, MD, aW, H, aaE, aF, aB, g, Eg, eE, t, f, eF, cm, acM, ar, tj, W, bb, dB, dJ, mx, U, mf, cbR, m, Gp, CW, L); #c1(PSEN2) c2(XP_005273256) c3(presenilin-2 isoform X1) c4(284385, 284386, 284387, 284388, 284389) c5(284390, 284391, 284392, 284393, 284394, 284395, 284396, 284397, 284398, 284399, 284400, 284401, 284402, 284403, 284404, 284405, 284406, 284407, 284408, 284409, 284410, 284411, 284412, 284413, 284414, 284415, 284416, 284417) c6( ) c7(CV, T, acL, cwt, aaH, ha, VT, aA, cz, aj, ci, il, fK, J, q, cd, ad, eA, mj, XX, di, cF, cc, WY, sO, rJ, Fo, Hk, aT, bK, K, hS, MO, H, ae, af, cwr, B, PP, MD, aW, eH, aaE, aF, aB, g, Eg, eE, t, f, eF, cm, acM, ar, tj, W, bb, dB, dJ, mx, U, mf, cbR, m, Gp, CW, L); #c1(PSENEN) c2(NP_001268461) c3(gamma-secretase subunit PEN-2) c4(99966) c5(99967, 99968, 99969, 99970, 99971, 99972, 99973, 99974, 99975, 99976) c6( ) c7(ciX, cm, cwu, af, L); #c1(PSG1) c2(NP_001171754) c3(pregnancy-specific beta-I-glycoprotein 1 isoform 2 precursor) c4(71499) c5(71500, 71501, 71502, 71503, 71504, 71505) c6( ) c7(ck, bM, T, bv, aE, eM, nO, Y, r, hD, uz, vx, q); #c1(PSG1) c2(NP_001171755) c3 (pregnancy-specific beta-1-glycoprotein 1 isoform 3 precursor) c4(71506, 71507) c5(71508, 71509, 71510, 71511, 71512, 71513) c6( ) c7(ck, bM, T, bv, aE, eM, nO, Y, r, hD, uz, vx, q); #c1(PSG1) c2(NP_001284702) c3(pregnancy-specific beta-1-glycoprotein 1 isoform 4 precursor) c4(112350) c5(112351, 112352, 112353, 112354, 112355, 112356, 112357) c6( ) c7(ck, bM, T, bv, aE, eM, nO, Y, r, hD, uz, vx, q); #c1(PSG1) c2(NP_008836) c3(pregnancy-specific beta-1-glycoprotein 1 isoform 1 precursor) c4(168945) c5(168946, 168947, 168948, 168949, 168950, 168951) c6( ) c7(ck, bM, T, bv, aE, eM, nO, Y, r, hD, uz, vx, q); #c1(PSG1) c2(XP_011525427) c3(pregnancy-specific beta-1-glycoprotein 1 isoform X2) c4( ) c5(319851, 319852, 319853, 319854, 319855) c6( ) c7(ck, bM, T, bv, aE, eM, nO, Y, r, hD, uz, vx, q); #c1(PSG2) c2(NP_112536) c3(pregnancy-specific beta-1-glycoprotein 2 precursor) c4(221337) c5(221338, 221339, 221340, 221341, 221342, 221343, 221344, 221345, 221346, 221347, 221348, 221349, 221350, 221351) c6( ) c7(T, dA, if, NT, aA, bB, n, ci, ze, qN, q, eW, x, Vp, bv, av, ib, aE, iu, aoc, nv, Qg, gd, aT, cU, dL, Tj, eL, De, B, ZH, BO, s, Jl, aW, CN, ff, r, aF, CQ, en, YF, cl, aoG, t, bz, g, cm, bP, eM, bE, dc, gX, m, X); #c1(PSG5) c2(NP_001123486) c3(pregnancy-specific beta-1-glycoprotein 5 precursor) c4(47160) c5(47161, 47162, 47163, 47164, 47165, 47166, 47167, 47168, 47169, 47170, 47171, 47172, 47173, 47174) c6( ) c7(wM, ZH, CN, cz, aq, qN, uC, B, alY); #c1(PSG6) c2(NP_001027020) c3(pregnancy-specific beta-1-glycoprotein 6 isoform b precursor) c4(30098) c5(30099, 30100, 30101, 30102, 30103, 30104) c6( ) c7(CN, ZH, aoc); #c1(PSG6) c2(NP_002773) c3(pregnancy-specific beta-1-glycoprotein 6 isoform a precursor) c4(131909) c5(131910, 131911, 131912, 131913, 131914, 131915) c6( ) c7(CN, ZH, aoc); #c1(PSG8) c2(NP_001123639) c3(pregnancy-specific beta-1-glycoprotein 8 isoform b precursor) c4(47924) c5(47925, 47926, 47927, 47928, 47929, 47930, 47931) c6( ) c7(CN, ZH, qN); #c1(PSG8) c2(NP_001123640) c3(pregnancy-specific beta-1-glycoprotein 8 isoform c) c4(47932) c5(47933, 47934, 47935, 47936, 47937, 47938) c6( ) c7(CN, ZH, qN); #c1(PSG8) c2(NP_874366) c3(pregnancy-specific beta-1-glycoprotein 8 isoform a precursor) c4(256629) c5(256630, 256631, 256632, 256633, 256634, 256635) c6( ) c7(CN, ZH, qN); #c1(PSG8) c2(XP_011525288) c3(pregnancy-specific beta-1-glycoprotein 8 isoform X1) c4(319745) c5(319746, 319747, 319748, 319749, 319750, 319751) c6( ) c7(CN, ZH, qN); #c1(PSG8) c2(XP_011525289) c3(pregnancy-specific beta-1-glycoprotein 8 isoform X2) c4(319752) [5( ) c6( ) c7(CN, ZH, qN); #c1(PSG8) c2(XP_011525290) c3(pregnancy-specific beta-1-glycoprotein 8 isoform X3) c4( ) c5(319753, 319754, 319755, 319756, 319757) c6( ) c7(CN, ZH, qN); #c1(PSG9) c2(NP_001288636) c3(pregnancy-specific beta-1-glycoprotein 9 isoform 2 precursor) c4(113895) c5(113896, 113897, 113898, 113899, 113900, 113901, 113902, 113903) c6( ) c7(bF); #c1(PSG9) c2(NP_001288638) c3(pregnancy-specific beta-1-glycoprotein 9 isoform 4 precursor) c4(113904) c5(113905, 113906, 113907, 113908, 113909, 113910, 113911, 113912) c6( ) c7(bF); #c1(PSG9) c2(NP_002775) c3(pregnancy-specific beta-1-glycoprotein 9 isoform 1 precursor) c4(131916, 131917) c5(131918, 131919, 131920, 131921, 131922, 131923, 131924, 131925, 131926, 131927, 131928, 131929, 131930, 131931, 131932, 131933) c6( ) c7(bF); #c1(PSG9) c2(XP_005259132) c3(pregnancy-specific beta-1-glycoprotein 9 isoform X1) c4(275958) c5(275959, 275960, 275961, 275962, 275963, 275964, 275965, 275966) c6( ) c7(bF); #c1(PSIP1) c2(NP_001121689) c3(PC4 and SFRS1-interacting protein isoform 2) c4(46057) c5(46058, 46059, 46060, 46061, 46062, 46063, 46064, 46065, 46066, 46067, 46068, 46069) c6( ) c7(bM, T, aC, fR, bO, aA, aj, ci, hF, eA, aR, amW, ad, aE, ec, aCJ, aql, dV, aT, h, dz, K, hS, zy, H, bYl, ae, WZ, jf, eG, LC, Y, aW, Ls, kD, aF, g, eE, t, cm, ck, OW, c, Jr, bE, Fi, eM, iT, m, bt, it, L); #c1(PSIP1) c2(NP_066967) c3(PC4 and SFRS1-interacting protein isoform 1) c4(207873) c5(207874, 207875, 207876, 207877, 207878, 207879, 207880, 207881, 207882, 207883, 207884) c6( ) c7(bM, T, aC, fR, bO, aA, aj, ci, hF, eA, aR, amW, ad, aE, ec, aCJ, aql, dV, aT, h, dz, K, hS, zy, H, bYI, ae, WZ, jf, eG, LC, Y, aW, Ls, KD, aF, g, eE, t, cm, ck, OW, c, Jr, bE, Fi, eM, iT, m, bt, it, L); #c1(PSIP1) c2(XP_011515999) c3(PC4 and SFRS1-interacting protein isoform X2) c4( ) c5(309207) c6( ) c7(bM, T, aC, fR, bO, aA, aj, ci, hF, eA, aR, amW, ad, aE, ec, aCJ, aql, dV, aT, h, dz, K, hS, zy, H, bYl, ae, WZ, jf, eG, LC, Y, aW, Ls, KD, aF, g, eE, t, cm, ck, OW, c, Jr, bE, Fi, eM, iT, m, bt, it, L); #c1(PSKH1) c2(NP_006733) c3(serine/threonine-protein kinase H1) c4(167547) c5(167548, 167549, 167550, 167551, 167552, 167553) c6( ) c7(ae, ad); #c1(PSMA1) c2(NP_001137409) c3(proteasome subunit alpha type-l isoform 3) c4(55642) c5(55643, 55644, 55645, 55646, 55647) c6( ) c7(T, aW, if, aT, qN, bw, bt); #c1(PSMA1) c2(NP_002777) c3(proteasome subunit alpha type-1 isoform 2) c4(131934) c5(131935, 131936, 131937, 131938, 131939, 131940, 131941, 131942, 131943, 131944, 131945) c6( ) c7(T, aW, if, aT, qN, bw, bt); #c1(PSMA1) c2(NP_683877) c3(proteasome subunit alpha type-1 isoform 1) c4(242609) c5(242610, 242611, 242612, 242613, 242614, 242615) c6( ) c7(T, aW, if, aT, qN, bw, bt); #c1(PSMA2) c2(NP_002778) c3(proteasome subunit alpha type-2) c4(131946, 131947) c5(131948, 131949, 131950, 131951, 131952, 131953, 131954, 131955, 131956, 131957, 131958) c6( ) c7(gZ, bA); #c1(PSMA3) c2(NP_002779) c3(proteasome subunit alpha type-3 isoform 1) c4(131959) c5(131960, 131961, 131962, 131963, 131964, 131965) c6( ) c7(vi); #c1(PSMA3) c2(NP_687033) c3 (proteasome subunit alpha type-3 isoform 2) c4(242644) c5(242645, 242646, 242647, 242648, 242649, 242650) c6( ) c7(vi); #c1(PSMA4) c2(NP_001096137) c3(proteasome subunit alpha type-4 isoform 1) c4(41088, 41089, 41090, 41091, 41092, 41093) c5(41094, 41095, 41096, 41097, 41098, 41099, 41100, 41101, 41102, 41103, 41104, 41105, 41106, 41107, 41108, 41109, 41110, 41111, 41112, 41113, 41114, 41115, 41116, 41117, 41118, 41119, 41120, 41121, 41122, 41123, 41124, 41125, 41126, 41127, 41128, 41129, 41130) c6( ) c7(av, T, zQ, ac, at, aq, h, ci, Vi, zP, bz, bc, y); #c1(PSMA4) c2(NP_001096138) c3(proteasome subunit alpha type-4 isoform 2) c4( ) c5(41131, 41132, 41133, 41134, 41135) c6( ) c7(av, T, zL, ac, at, aq, h, ci, Vi, zP, bz, bc, y); #c1(PSMA6) c2(NP_001269161) c3(proteasome subunit alpha type-6 isoform b) c4( ) c5(100746, 100747, 100748, 100749, 100750) c6( ) c7(bL, T, dD, ea, aw, jh, cz, fd, cl, aj, j, eq, J, wD, rS, M, hD, h, eN, eu, cW, cr, ac, ei); #c1(PSMA6) c2(NP_001269163) c3(proteasome subunit alpha type-6 isoform c) c4(100751, 100752) c5(100753, 100754, 100755, 100756, 100757, 100758) c6( ) c7(bL, T, dD, ea, aw, jh, cz, fd, cl, aj, j, eq, J, wD, rS, M, hD, h, eN, eu, cW, cr, ac, ei); #c1(PSMA6) c2(NP_002782) c3(proteasome subunit alpha type-6 isoform a) c4(131966, 131967, 131968, 131969, 131970) c5(131971, 131972, 131973, 131974, 131975, 131976, 131977, 131978, 131979, 131980, 131981, 131982, 131983, 131984, 131985, 131986, 131987, 131988, 131989, 131990, 131991, 131992, 131993, 131994, 131995, 131996, 131997, 131998, 131999, 132000, 132001) c6( ) c7(bL, T, dD, ea, aw, jh, cz, fd, cl, aj, j, eq, J, wD, rS, M, hD, h, eN, eu, cW, cr, ac, ei); #c1(PSMA7) c2(NP_002783) c3(proteasome subunit alpha type-7) c4(132002, 132003, 132004, 132005) c5(132006, 132007, 132008, 132009, 132010, 132011, 132012, 132013, 132014, 132015, 132016, 132017, 132018, 132019, 132020, 132021, 132022, 132023, 132024, 132025, 132026, 132027, 132028, 132029) c6( ) c7(h, ol, lo, q, r); #c1(PSMB1) c2(NP_002784) c3(proteasome subunit beta type-1) c4(132030, 132031) c5(132032, 132033, 132034, 132035, 132036, 132037, 132038, 132039, 132040, 132041, 132042, 132043) c6( ) c7(A, bX); #c1(PSMB10) c2(NP_002792) c3(proteasome subunit beta type-10 precursor) c4(132185) c5(132186, 132187, 132188, 132189, 132190, 132191) c6( ) c7(T, aW, Dq, aT, h, fd, X); #c1(PSMB4) c2(NP_002787) c3(proteasome subunit beta type-4) c4(132044) c5(132045, 132046, 132047, 132048, 132049, 132050) c6( ) c7(T, ac, bO, h, Z, eA); #c1(PSMB5) c2(NP_001124197) c3(proteasome subunit beta type-5 isoform 2) c4( ) c5(48510, 48511, 48512, 48513, 48514) c6( ) c7(x, T, amS, pe, j, k, y); #c1(PSMB5) c2(NP_001138404) c3(proteasome subunit beta type-5 isoform 3) c4(56570) c5(56571, 56572, 56573, 56574, 56575) c6( ) c7(x, T, amS, pe, j, k, y); #c1(PSMB5) c2(NP_002788) c3(proteasome subunit beta type-5 isoform 1) c4(132051, 132052) c5(132053, 132054, 132055, 132056, 132057, 132058, 132059, 132060) c6( ) c7(x, T, amS, pe, j, k, y); #c1(PSMB6) c2(NP_002789) c3(proteasome subunit beta type-6 isoform 1 proprotein) c4(132061) c5(132062, 132063, 132064, 132065, 132066, 132067) c6( ) c7(bL, bM, T, bfY, jh, bB, bS, bck, ci, fu, v, fr, bfU, hF, hX, x, pp, iX, aql, kH, h, hn, RG, iU, xR, Y, bfV, eZ, hp, bw, bz, bfW, hq, ck, qJ, BU, cA, avS, bE, iT, aDs, cj, bfX, k, pw, y, bt, EB); #c1(PSMB7) c2(NP_002790) c3(proteasome subunit beta type-7 proprotein) c4(132068, 132069, 132070) c5(132071, 132072, 132073, 132074, 132075, 132076, 132077, 132078, 132079, 132080, 132081, 132082, 132083, 132084, 132085, 132086, 132087, 132088) c6( ) c7(aW, X, ac, bX, aT); #c1(PSMB8) c2(NP_004150) c3(proteasome subunit beta type-8 isoform E1 proprotein) c4(143881) c5(143882, 143883, 143884, 143885, 143886, 143887, 143888, 143889, 143890, 143891, 143892, 143893, 143894, 143895, 143896, 143897, 143898, 143899, 143900, 143901, 143902, 143903, 143904, 143905, 143906, 143907, 143908, 143909) c6( ) c7(bM, T, Dw, FR, if, jx, fN, sJ, cz, n, cl, bB, qj, fr, qN, V, q, o, eW, hX, wD, auv, bvG, F, ld, cu, HD, Ds, ey, hu, Df, af, ul, A, mR, asD, hl, cr, eZ, BE, bQk, GR, dr, Zj, ly, ig, bE, dc, aCl, bQm, y, X); #c1(PSMB8) c2(NP_683720) c3(proteasome subunit beta type-8 isoform EZ proprotein) c4(242447, 242448, 242449, 242450) c5(242451, 242452, 242453, 242454, 242455, 242456, 242457, 242458, 242459, 242460, 242461, 242462, 242463, 242464, 242465, 242466, 242467, 242468, 242469, 242470, 242471, 242472, 242473, 242474, 242475, 242476, 242477, 242478, 242479, 242480, 242481, 242482, 242483, 242484, 242485, 242486, 242487, 242488, 242489, 242490, 242491, 242492, 242493, 242494, 242495, 242496, 242497, 242498, 242499, 242500, 242501, 242502, 242503, 242504, 242505, 242506, 242507, 242508, 242509, 242510, 242511, 242512, 242513, 242514, 242515) c6( ) c7(bM, T, Dw, fR, if, jx, fN, sJ, cz, n, cl, bB, qj, fr, qN, V, q, o, eW, hX, wD, auv, bvG, F, ld, cU, hD, Ds, ey, hu, Df, af, ul, A, mR, asD, hl, cr, eZ, eE, bQk, GR, dr, Zj, ly, ig, bE, dc, aCl, bQm, y, X); #c1(PSMB9) c2(NP_002791) c3(proteasome subunit beta type-9 proprotein) c4(132089, 132090, 132091, 132092, 132093, 132094) c5(132095, 132096, 132097, 132098, 132099, 132100, 132101, 132102, 132103, 132104, 132105, 132106, 132107, 132108, 132109, 132110, 132111, 132112, 132113, 132114, 132115, 132116, 132117, 132118, 132119, 132120, 132121, 132122, 132123, 132124, 132125, 132126, 132127, 132128, 132129, 132130, 132131, 132132, 132133, 132134, 132135, 132136, 132137, 132138, 132139, 132140, 132141, 132142, 132143, 132144, 132145, 132146, 132147, 132148, 132149, 132150, 132151, 132152, 132153, 132154, 132155, 132156, 132157, 132158, 132159, 132160, 132161, 132162, 132163, 132164, 132165, 132166, 132167, 132168, 132169, 132170, 132171, 132172, 132173, 132174, 132175, 132176, 132177, 132178, 132179, 132180, 132181, 132182, 132183, 132184) c6( ) c7(T, if, aw, ex, iW, jh, cz, Bm, cl, fr, ci, qN, V, aYs, q, o, ad, ai, qW, hX, wD, amg, rw, ld, dV, aT, cU, hD, eV, ja, ey, ae, af, A, mR, ZH, CN, aW, Y, asD, cr, r, eZ, g, eE, t, Zj, op, ig, Zg, dc, vA, bo, aiM, aCl, ol, y, X); #c1(PSMB9) c2(XP_011544619) c3 (proteasome subunit beta type-9 isoform X1) c4(343607, 343608, 343609) c5(343610, 343611, 343612, 343613, 343614, 343615, 343616, 343617, 343618, 343619, 343620, 343621, 343622, 343623, 343624, 343625, 343626, 343627, 343628, 343629, 343630, 343631, 343632, 343633, 343634, 343635, 343636, 343637, 343638, 343639, 343640, 343641, 343642, 343643, 343644, 343645, 343646, 343647, 343648, 343649, 343650, 343651, 343652, 343653, 343654, 343655, 343656, 343657, 343658, 343659, 343660, 343661, 343662, 343663, 343664, 343665, 343666, 343667, 343668) c6( ) c7(T, if, aw, ex, iW, jh, cz, Bm, cl, fr, ci, qN, V, aYs, q, o, ad, ai, qW, hX, wD, amg, rw, ld, dV, aT, cu, hD, eV, ja, ey, ae, af, A, mR, ZH, CN, aW, Y, asD, cr, r, eZ, g, eE, t, Zj, op, ig, Zg, dc, vA, bo, aiM, aCl, oQ, y, X); #c1 (PSMC1) c2(NP_002793) c3(26S protease regulatory subunit 4) c4(132192, 132193) c5(132194, 132195, 132196, 132197, 132198, 132199, 132200, 132201, 132202, 132203, 132204, 132205) c6( ) c7(bC, aCp, U, Be); #c1(PSMC2) c2(NP_001191382) c3(26S protease regulatory subunit 7 isoform 2) c4( ) c5(81480, 81481, 81482, 81483, 81484, 81485, 81486, 81487, 81488, 81489) c6( ) c7(Vi); #c1(PSMC2) c2(NP_002794) c3(26S protease regulatory subunit 7 isoform 1) c4(132206, 132207, 132208) c5(132209, 132210, 132211, 132212, 132213, 132214, 132215, 132216, 132217, 132218, 132219, 132220, 132221, 132222, 132223, 132224, 132225) c6( ) c7(Vi); #c1(PSMC3) c2(NP_002795) c3(26S protease regulatory subunit 6A) c4(132226) c5(132227, 132228, 132229, 132230, 132231, 132232) c6( ) c7(h); #c1(PSMC31P) c2(NP_001242943) c3(homologous-pairing protein 2 homolog isoform 3) c4( ) c5(89336, 89337, 89338, 89339, 89340) c6( ) c7(zZ, il, vH, cw, m, avr, t, ad); #c1(PSMC3IP) c2(NP_037422) c3(homologous-pairing protein 2 homolog isoform 1) c4(175474) c5(175475, 175476, 175477, 175478, 175479, 175480) c6( ) c7(zZ, il, vH, cw, m, avr, t, ad); #c1(PSMC3IP) c2(NP_057640) c3(homologous-pairing protein 2 homolog isoform 2) c4(192002, 192003) c5(192004, 192005, 192006, 192007, 192008, 192009, 192010) c6( ) c7(zZ, il, vH, cw, m, avr, t, ad); #c1(PSMC4) c2(NP_006494) c3(26S protease regulatory subunit GB isoform 1) c4(165710) c5(165711, 165712, 165713, 165714, 165715, 165716, 165717, 165718) c6( ) c7(hl, aT, fr, K, an, Xh, eW); #c1(PSMC4) c2(NP_694546) c3(26S protease regulatory subunit 6B isoform 2) c4(245111) c5(245112, 245113, 245114, 245115, 245116, 245117) c6( ) c7(hl, aT, fr, K, an, Xh, eW); #c1(PSMC5) c2(NP_001186092) c3(26S protease regulatory subunit 8 isoform 2) c4( ) c5(77115, 77116, 77117, 77118, 77119, 77120, 77121, 77122) c6( ) c7(dD, ea, aT, h, eq, zy, cm); #c1(PSMC5) c2(NP_002796) c3(26S protease regulatory subunit 8 isoform 1) c4(132233) c5(132234, 132235, 132236, 132237, 132238, 132239, 132240, 132241, 132242) c6( ) c7(dD, ea, aT, h, eq, zy, cm); #c1(PSMC5) c2(XP_006722043) c3(26S protease regulatory subunit 8 isoform X1) c4(294559) c5( ) c6( ) c7(dD, ea, al, h, eq, zy, cm); #c1(PSMC6) c2(NP_002797) c3(26S protease regulatory subunit 108) c4(132243) c5(132244, 132245, 132246, 132247, 132248, 132249) c6( ) c7(G, T, dA, yw, kB, wu, kf, h, wm, bu, ae, ad, VF); #c1(PSMD1) c2(NP_001177966) c3(26S proteasome non-ATPase regulatory subunit 1 isoform 2) c4(73243) c5(73244, 73245, 73246, 73247, 73248, 73249, 73250, 73251, 73252, 73253) c6( ) c7(T, aW, ff, U, aT, en); #c1(PSMD1) c2(NP_002798) c3(26S proteasome non-ATPase regulatory subunit 1 isoform 1) c4(132250) c5(132251, 132252, 132253, 132254, 132255, 132256) c6( ) c7(T, aW, ff, U, aT, en); #c1(PSMD10) c2(NP_002805) c3(26S proteasome non-ATPase regulatory subunit 10 isoform 1) c4(132348, 132349, 132350) c5(132351, 132352, 132353, 132354, 132355, 132356, 132357, 132358, 132359, 132360, 132361, 132362, 132363, 132364, 132365, 132366, 132367, 132368, 132369) c6( ) c7(Mb, T, fM, aA, aj, ci, q, J, hX, jz, aE, aT, K, hu, je, s, qt, aW, ff, r, aq, en, cl, iR, iM, anT, Be, hL, np, B); #c1(PSMD10) c2(NP_736606) c3(26S proteasome non-ATPase regulatory subunit 10 isoform 2) c4(247317, 247318) c5(247319, 247320, 247321, 247322, 247323, 247324, 247325, 247326, 247327, 247328, 247329) c6( ) c7(Mb, T, fM, aA, aj, ci, q, J, hX, jz, aE, aT, K, hu, je, s, qt, aW, ff, r, aq, en, cl, iR, iM, anT, Be, hl, np, B); #c1(PSMD12) c2(NP_002807) c3(26S proteasome non-ATPase regulatory subunit 12 isoform 1) c4(132370) c5(132371, 132372, 132373, 132374, 132375, 132376) c6( ) c7(A, F, xE, dV, h, y, bt, amW, ul); #c1(PSMD12) c2(NP_777360) c3(26S proteasome non-ATPase regulatory subunit 12 isoform 2) c4(251087) c5(251088, 251089, 251090, 251091, 251092, 251093) c6( ) c7(A, F, xE, dV, h, y, bt, amW, uQ); #c1(PSMD13) c2(NP_002808) c3(26S proteasome non-ATPase regulatory subunit 13 isoform 1) c4(132377) c5(132378, 132379, 132380, 132381, 132382, 132383, 132384, 132385, 132386, 132387, 132388, 132389) c6( ) c7(h, lo); #c1(PSMD13) c2(NP_787128) c3(26S proteasome non-ATPase regulatory subunit 13 isoform 2) c4(252143, 252144) c5(252145, 252146, 252147, 252148, 252149, 252150) c6( ) c7(h, lo); #c1(PSMD14) c2(NP_005796) c3(26S proteasome non-ATPase regulatory subunit 14) c4(159538, 159539) c5(159540, 159541, 159542, 159543, 159544, 159545, 159546, 159547, 159548, 159549, 159550, 159551) c6( ) c7(y, j); #c1(PSMD2) c2(NP_001265638) c3(26S proteasome non-ATPase regulatory subunit 2 isoform 3) c4(99070) c5(99071, 99072, 99073, 99074, 99075) c6( ) c7(T, if, bs, jE, ci, qN, V, br, hX, oj, aU, K, af, d, Y, g, bw, bz, ck, bb, c, Kv, U, bt, B); #c1(PSMD2) c2(NP_002799) c3(26S proteasome non-ATPase regulatory subunit 2 isoform 1) c4(132257, (32258) c5(132259, 132260, 132261, 132262, 132263, 132264, 132265, 132266, 132267, 132268, 132269, 132270, 132271) c6( ) c7(T, if, bs, jE, ci, qN, V, br, hX, oj, aU, K, af, d, Y, g, bw, bz, ck, bb, c, Kv, U, bt, B); #c1(PSMD3) c2(NP_002800) c3(26S proteasome non-ATPase regulatory subunit 3) c4(132272, 132273, 132274, 132275) c5(132276, 132277, 132278, 132279, 132280, 132281, 132282, 132283, 132284, 132285, 132286, 132287, 132288, 132289, 132290, 132291, 132292, 132293, 132294, 132295, 132296, 132297, 132298, 132299) c6( ) c7(h, oZ, os); #c1(PSMD4) c2(NP_002801) c3(26S proteasome non-ATPase regulatory subunit 4) c4(132300) c5(132301, 132302, 132303, 132304, 132305, 132306, 132307, 132308, 132309, 132310, 132311, 132312) c6( ) c7(T, if, qN, V, av, Mo, lg, ds, aT, kf, on, DA, iU, aW, ff, ahk, lb, lo, en, cm, bN, es, eM, iA); #c1(PSMD4) c2(XP_005245411) c3(26S proteasome non-ATPase regulatory subunit 4 isoform X1) c4(266513, 266514) c5(266515, 266516, 266517, 266518, 266519, 266520) c6( ) c7(T, if, qN, V, av, Mo, lg, ds, aT, kf, on, DA, iU, aW, ff, ahk, lb, lo, en, cm, bN, es, eM, iA); #c1(PSMD6) c2(NP_001258708) c3(26S proteasome non-ATPase regulatory subunit 6 isoform 1) c4(96397) c5(96398, 96399, 96400, 96401, 96402, 96403) c6( ) c7(bL, ac, Dy, aW, Y, aT, h); #c1(PSMD6) c2(NP_001258709) c3(26S proteasome non-ATPase regulatory subunit 6 isoform 3) c4(96404) c5(96405, 96406, 96407, 96408, 96409, 96410) c6( ) c7(bL, ac, Dy, aW, Y, aT, h); #c1(PSMD6) c2(NP_055629) c3(26S proteasome non-ATPase regulatory subunit 6 isoform 2) c4(182359) c5(182360, 182361, 182362, 182363, 182364, 182365) c6( ) c7(bL, ac, Dy, aW, Y, aT, h); #c1(PSMD7) c2(NP_002802) c3(26S proteasome non-ATPase regulatory subunit 7) c4(132313, 132314) c5(132315, 132316, 132317, 132318, 132319, 132320, 132321, 132322, 132323, 132324, 132325, 132326) c6( ) c7(bM, Hn, V, q, ad, dV, cU, h, ae, A, Y, afS, cr, r, eZ, fe, eE, Jr, z, eo, afT, y); #c1(PSMD8) c2(NP_002803) c3(26S proteasome non-ATPase regulatory subunit 8) c4(132327) c5(132328, 132329, 132330, 132331, 132332, 132333) c6( ) c7(ib, aW, U, aT, h, v, hX); #c1(PSMD9) c2(NP_002804) c3(26S proteasome non-ATPase regulatory subunit 9 isoform 1) c4(132334, 132335) c5(132336, 132337, 132338, 132339, 132340, 132341, 132342, 132343, 132344, 132345, 132346, 132347) c6( ) c7(T, ea, if, fd, bB, ci, aE, iI, Qk, K, eL, bc, xQ, yo, aW, as, cm, bw, GR, eM, nB, dc, go, vA, cj, m, dD, NT, n, aj, fr, ct, qN, jz, h, dL, mJ, ge, aq, zZ, hq, iO, kT, bP, yP, hR, it, B, bL, fM, aoE, aA, cz, v, j, q, J, eA, XX, aT, dz, bK, ae, af, jf, s, iU, Y, r, en, cl, t, ik, aEp, ck, nM, iM, dy, bt, On, hS, hl, Ke, pY, jh, bS, eq, ad, hX, hd, ib, fb, d, je, et, cH, ff, apb, aF, Z, bz, bE, aS, EB); #c1(PSME1) c2(NP_006254) c3(proteasome activator complex subunit 1 isoform 1) c4(163314) c5(163315, 163316, 163317, 163318, 163319, 163320) c6( ) c7(zP, zQ, Dq, h, ci, t, it, X); #c1(PSME1) c2(NP_788955) c3(proteasome activator complex subunit 1 isoform 2) c4(252181) c5(252182, 252183, 252184, 252185, 252186, 252187) c6( ) c7(zP, zQ, Dq, h, ci, t, it, X); #c1(PSME2) c2(NP_002809) c3(proteasome activator complex subunit 2) c4(132390) c5(132391, 132392, 132393, 132394, 132395, 132396) c6( ) c7(T, JI, aA, h, fd, g, it, X); #c1(PSME3) c2(NP_001253974) c3(proteasome activator complex subunit 3 isoform 3) c4( ) c5(94341, 94342) c6( ) c7(T, B, eM, jx, fc, eZ, h, np, fL, cP, bC, ey, mC); #c1(PSME3) c2(NP_005780) c3(proteasome activator complex subunit 3 isoform 1) c4(159282, 159283, 159284) c5(159285, 159286, 159287, 159288, 159289, 159290, 159291, 159292, 159293, 159294, 159295, 159296, 159297, 159298, 159299) c6( ) c7(T, B, eM, jx, fc, eZ, h, np, fl, cP, bC, ey, mC); #c1(PSME3) c2(NP_789839) c3(proteasome activator complex subunit 3 isoform 2) c4(252349) c5(252350, 252351, 252352, 252353, 252354, 252355, 252356, 252357, 252358) c6( ) c7(T, B, eM, jx, fc, eZ, h, np, fl, cP, bC, ey, mC); #c1(PSME3) c2(XP_011522480) c3(proteasome activator complex subunit 3 isoform X1) c4( ) c5(317114, 317115, 317116, 317117, 317118, 317119) c6( ) c7(T, B, eM, jx, fc, eZ, h, np, fl, cP, bC, ey, mC); #c1(PSME4) c2(NP_055429) c3(proteasome activator complex subunit 4) c4(181085) c5(181086, 181087, 181088, 181089, 181090, 181091, 181092, 181093, 181094, 181095, 181096, 181097) c6( ) c7(h); #c1(PSME4) c2(XP_006712032) c3(proteasome activator complex subunit 4 isoform X6) c4(286353) c5( ) c6( ) c7(h); #c1(PSMF1) c2(NP_848693) c3(proteasome inhibitor PI31 subunit) c4(254158, 254159, 254160) c5(254161, 254162, 254163, 254164, 254165, 254166, 254167, 254168, 254169, 254170, 254171, 254172, 254173, 254174, 254175, 254176) c6( ) c7(ac); #c1(PSMF1) c2(XP_005260930) c3(proteasome inhibitor PI31 subunit isoform X3) c4(276984, 276985, 276986, 276987) c5( ) c6( ) c7(ac); #c1(PSMF1) c2(XP_006723727) c3(proteasome inhibitor PI31 subunit isoform X2) c4( ) c5(295330, 295331, 295332, 295333, 295334, 295335, 295336, 295337, 295338, 295339, 295340, 295341, 295342, 295343, 295344, 295345, 295346, 295347, 295348, 295349, 295350, 295351, 295352, 295353, 295354, 295355, 295356, 295357, 295358, 295359) c6( ) c7(ac); #c1(PSMG1) c2(NP_003711) c3(proteasome assembly chaperone 1 isoform a) c4(140351) c5(140352, 140353, 140354, 140355, 140356, 140357) c6( ) c7(ul, fd, Fb, uu, hF, Fc, eA, ad, pX, bK, K, he, ae, fe, kp, iV, cm, bz, bP, Ei, m, pk); #c1(PSMG1) c2(NP_982257) c3(proteasome assembly chaperone 1 isoform b) c4(263227) c5(263228, 263229, 263230, 263231, 263232, 263233) c6( ) c7(ul, fd, Fb, uu, hF, Fc, eA, ad, pX, bK, K, he, ae, fe, kp, iV, cm, bz, bP, Ei, m, pk); #c1(PSMG2) c2(NP_064617) c3(proteasome assembly chaperone 2 isoform 1) c4(203306, 203307) c5(203308, 203309, 203310, 203311, 203312, 203313, 203314, 203315, 203316, 203317, 203318, 203319) c6( ) c7(A, eZ, aq, K, bw, eW); #c1(PSMG3) c2(XP_011513881) c3(proteasome assembly chaperone 3 isoform X1) c4(306295) c5(306296, 306297, 306298, 306299, 306300, 306301, 306302, 306303, 306304, 306305, 306306, 306307, 306308, 306309) c6( ) c7(bji, Ws); #c1(PSORS1C1) c2(NP_054787) c3(psoriasis susceptibility 1 candidate gene 1 protein) c4(177840, 177841, 177842, 177843) c5(177844, 177845, 177846, 177847, 177848, 177849, 177850, 177851, 177852, 177853, 177854, 177855, 177856, 177857, 177858, 177859, 177860, 177861, 177862, 177863, 177864, 177865, 177866, 177867, 177868, 177869, 177870, 177871, 177872, 177873, 177874, 177875, 177876, 177877, 177878, 177879) c6( ) c7(xS, AW, iQ, ua, od, cr, eZ, gA, lf, V, pb, wD, hH); #c1(PSORS1C2) c2(NP_054788) c3(psoriasis susceptibility 1 candidate gene 2 protein precursor) c4(177880, 177881) c5(177882, 177883, 177884, 177885, 177886, 177887, 177888, 177889, 177890, 177891, 177892, 177893, 177894, 177895, 177896, 177897, 177898, 177899, 177900, 177901, 177902, 177903, 177904, 177905, 177906, 177907, 177908, 177909, 177910, 177911, 177912, 177913, 177914, 177915, 177916, 177917, 177918, 177919, 177920, 177921, 177922, 177923, 177924, 177925, (77926) c6(177927, 177928) c7(T, Jr, aW, cr, aT, eZ, fd, hH); #c1(PSPC1) c2(XP_006719907) c3(paraspeckle component 1 isoform X1) c4(293068, 293069) c5(293070, 293071, 293072, 293073, 293074, 293075, 293076, 293077, 293078, 293079, 293080, 293081) c6( ) c7(act); #c1(PSPC1) c2(XP_011533442) c3(paraspeckle component 1 isoform X4) c4(329560) c5( ) c6( ) c7(act); #c1(PSPC1) c2(XP_011533445) c3(paraspeckle component 1 isoform X7) c4( ) c5(329561, 329562, 329563, 329564, 329565, 329566) c6( ) c7(act); #c1(PSPH) c2(XP_011513763) c3(phosphoserine phosphatase isoform X1) c4(306132, 306133) c5(306134, 306135, 306136, 306137, 306138, 306139, 306140, 306141, 306142, 306143, 306144, 306145, 306146, 306147, 306148, 306149, 306150, 306151, 306152, 306153, 306154, 306155, 306156, 306157, 306158) c6( ) c7(ar, cww, ctY, kp, act, ae, rL, ad); #c1(PSPH) c2(XP_011513764) c3(phosphoserine phosphatase isoform X3) c4(306159) c5(306160, 306161, 306162, 306163, 306164, 306165, 306166, 306167, 306168, 306169) c6( ) c7(ar, cww, ctY, kp, act, ae, rl, ad); #c1(PSPN) c2(NP_004149) c3(persephin precursor) c4(143874) c5(143875, 143876, 143877, 143878, 143879, 143880) c6( ) c7(bxy, ahE, ar, kp, bK, ahl, act, ae, ad, ek); #c1(PSTPIP1) c2(NP_003969) c3(proline-serine-threonine phosphatase-interacting protein 1) c4(142516, 142517) c5(142518, 142519, 142520, 142521, 142522, 142523, 142524, 142525, 142526, 142527, 142528, 142529) c6( ) c7(y, dE, qY, ih, dN, fe, cL, bQL, hH, NX, o, wD); #c1(PSTPIP2) c2(NP_077748) c3(proline-serine-threonine phosphatase-interacting protein 2) c4(215379, 215380) c5(215381, 215382, 215383, 215384, 215385, 215386, 215387, 215388, 215389, 215390, 215391, 215392) c6( ) c7(azi, cai, ey); #c1(PTBP1) c2(NP_002810) c3(polypyrimidine tract-binding protein 1 isoform a) c4(132397) c5(132398, 132399, 132400, 132401, 132402, 132403) c6( ) c7(bM, T, aC, fR, bB, TM, Vd, ci, aGX, lR, aR, aE, SV, dV, K, brV, zL, aW, Ku, cm, QA, eM, mx, U, QF, m, xh, n, fr, nA, zP, eW, ft, Ak, h, dx, aq, SE, dB, Qu, rx, arg, B, jx, aA, tc, eA, WY, aT, kf, an, ae, af, vT, Y, aaE, en, t, pb, bN, ck, rR, kP, c, ig, dh, ha, jh, hF, ad, hX, ds, oj, fb, iL, d, ff, eZ, nf, aF, iR, bz, dr, dR, bE, anY, aEs, y); #c1(PTBP1) c2(NP_114367) c3(polypyrimidine tract-binding protein 1 isoform b) c4(222504) c5(222505, 222506, 222507, 222508, 222509, 222510) c6( ) c7(bM, T, aC, fR, bB, TM, Vd, ci, aGX, lR, aR, aE, SV, dV, K, brV, zQ, aW, Ku, cm, QA, eM, mx, U, QF, m, xh, n, fr, nA, zP, eW, ft, Ak, h, dx, aq, SE, dB, Qu, rx, arg, B, jx, aA, tc, eA, WY, aT, kf, an, ae, af, vT, Y, aaE, en, t, pb, bN, ck, rR, KP, c, ig, dh, ha, jh, hF, ad, hX, ds, oj, fb, iL, d, ff, eZ, nf, aF, iR, bz, dr, dR, bE, anY, aEs, y); #c1(PTBP1) c2(NP_114368) c3(polypyrimidine tract-binding protein 1 isoform c) c4(222511) c5(222512, 222513, 222514, 222515, 222516, 222517, 222518, 222519, 222520, 222521, 222522, 222523) c6( ) c7(bM, T, aC, fR, bB, TM, Vd, ci, aGX, lR, aR, aE, SV, dV, K, brV, zQ, aW, Ku, cm, QA, eM, mx, U, QF, m, xh, n, fr, nA, zP, eW, ft, Ak, h, dx, aq, SE, dB, Qu, rx, arg, B, jx, aA, tc, eA, WY, aT, kf, an, ae, af, vT, Y, aaE, en, t, pb, bN, ck, rR, kP, c, ig, dh, ha, jh, hF, ad, hX, ds, oj, fb, iL, d, ff, eZ, nf, aF, iR, bz, dr, dR, bE, anY, aEs, y); #c1(PTBP1) c2(XP_005259655) c3(polypyrimidine tract-binding protein 1 isoform X2) c4(276221) c5( ) c6( ) c7(bM, T, aC, fR, bB, TM, Vd, ci, aGX, lR, aR, aE, SV, dV, K, brV, zL, aW, Ku, cm, DA, eM, mx, U, QF, m, xh, n, fr, nA, zP, eW, ft, Ak, h, dx, aq, SE, dB, Qu, rx, arg, B, jx, aA, tc, eA, WY, aT, kf, an, ae, af, vT, Y, aaE, en, t, pb, bN, ck, rR, kP, c, ig, dh, ha, jh, hF, ad, hX, ds, oj, fb, iL, d, ff, eZ, nf, aF, iR, bz, dr, dR, bE, anY, aEs, y); #c1(PTBP2) c2(NP_001287914) c3(polypyrimidine tract-binding protein 2 isoform 1) c4(113199, 113200, 113201) c5(113202, 113203, 113204, 113205, 113206, 113207) c6( ) c7(bxy, Hx, cwx, pN, aW, anY, ig, jx, aT, cm, n, h, K, m, eA, act, t, af); #c1(PTBP2) c2(NP_001287916) c3(polypyrimidine tract-binding protein 2 isoform 3) c4(113208) c5(113209, 113210, 113211, 113212, 113213, 113214) c6( ) c7(bxy, Hx, cwx, pN, aW, anY, ig, jx, aT, cm, n, h, K, m, eA, act, t, af); #c1(PTBP2) c2(NP_001287917) c3(polypyrimidine tract-binding protein 2 isoform 4) c4(113215) c5(113216, 113217, 113218, 113219, 113220, 113221, 113222, 113223, 113224, 113225, 113226) c6( ) c7(bxy, Hx, cwx, pN, aW, anY, ig, jx, aT, cm, n, h, K, m, eA, act, t, af); #c1(PTBP2) c2(NP_001287918) c3(polypyrimidine tract-binding protein 2 isoform 5) c4(113227) c5(113228, 113229, 113230, 113231, 113232, 113233) c6( ) c7(bxy, Hx, cwx, pN, aW, anY, ig, jx, aT, cm, n, h, K, m, eA, act, t, af); #c1(PTBP2) c2(NP_001287919) c3(polypyrimidine tract-binding protein 2 isoform 7) c4( ) c5(113234, 113235, 113236, 113237, 113238, 113239) c6( ) c7(bxy, Hx, cwx, pN, aW, anY, ig, jx, al, cm, n, h, K, m, eA, act, t, af); #c1(PTBP2) c2(XP_011540177) c3(polypyrimidine tract-binding protein 2 isoform X2) c4(338554) c5(338555) c6( ) c7(bxy, Hx, cwx, pN, aW, anY, ig, jx, aT, cm, n, h, K, m, eA, act, t, af); #c1(PTBP2) c2(XP_011540178) c3(polypyrimidine tract-binding protein 2 isoform X3) c4( ) c5(338556) c6( ) c7(bxy, Hx, cwx, pN, aW, anY, ig, jx, aT, cm, n, h, K, m, eA, act, t, af); #c1(PTCD1) c2(NP_056360) c3 (pentatricopeptide repeat-containing protein 1, mitochondrial) c4(187275, 187276, 187277, 187278, 187279, 187280) c5(187281, 187282, 187283, 187284, 187285, 187286, 187287, 187288, 187289, 187290, 187291, 187292, 187293, 187294, 187295, 187296, 187297, 187298, 187299, 187300, 187301, 187302, 187303, 187304, 187305, 187306, 187307, 187308, 187309, 187310, 187311) c6( ) c7(oe); #c1(PTCH1) c2(NP_000255) c3(protein patched homolog 1 isoform L) c4(5832, 5833, 5834) c5(5835, 5836, 5837, 5838, 5839, 5840, 5841, 5842, 5843, 5844, 5845, 5846) c6( ) c7(T, aC, aCh, rV, cwz, fd, ci, aon, xY, bLg, Na, Vp, bv, xy, aE, iv, K, eL, aa, aCg, aW, Az, CT, cwB, Qm, g, cm, aAW, qJ, bDd, eM, nB, vA, cj, m, asW, aAz, n, fr, LI, aCi, azj, amJ, Nn, cwC, h, dL, Gc, dx, cC, cml, aua, aq, kp, alf, fq, op, iO, asl, dB, hk, Dp, YH, bkE, B, AN, ek, ZZ, AS, yk, fZ, aA, hz, Do, AO, q, er, eA, eJ, XX, nv, Qg, afQ, aT, aAn, Nc, Fx, jf, iF, s, Qr, Y, ayU, r, en, t, Px, f, ik, ck, sS, nM, auo, cwA, hS, kh, id, Dd, dG, cwy, ad, dP, hd, Db, yF, Yj, d, ff, Xj, aF, bF, es, Lj, cwD, adg, ZW, jr, bsJ); #c1(PTCH1) c2(NP_001077071) c3(protein patched homolog 1 isoform M) c4(37652, 37653, 37654) c5(37655, 37656, 37657, 37658, 37659, 37660, 37661, 37662, 37663, 37664, 37665, 37666, 37667, 37668, 37669, 37670) c6( ) c7(T, aC, aCh, rV, cwz, fd, ci, aon, xY, bLg, Na, Vp, bv, xy, aE, iv, K, eL, aa, aCg, aW, Az, CT, cwB, Qm, g, cm, aAW, qJ, bDd, eM, nB, vA, cj, m, asW, aAz, n, fr, LI, aCi, azj, amJ, Nn, cwC, h, dL, Gc, dx, cC, cml, aua, aq, kp, alf, fq, op, iO, asl, dB, hk, Dp, YH, bkE, B, AN, ek, ZZ, AS, yk, fZ, aA, hz, Lo, AO, q, er, eA, eJ, XX, nv, Qg, afQ, aT, aAn, Nc, Fx, jf, if, s, Ar, Y, ayU, r, en, t, Px, f, ik, ck, sS, nM, auo, cwA, hS, kh, id, Dd, dG, cwy, ad, dP, hd, Db, yF, Yj, d, ff, Xj, aF, bF, es, Lj, cwD, adg, ZW, jr, bsJ); #c1(PTCH1) c2(NP_001077072) c3(protein patched homolog 1 isoform L′) c4(37671) c5(37672, 37673, 37674, 37675, 37676, 37677, 37678, 37679) c6( ) c7(T, aC, aCh, rV, cwz, fd, ci, aon, xY, bLg, Na, Vp, bv, xy, aE, iv, K, eL, aa, aCg, aW, Az, CT, cwB, Qm, g, cm, aAW, qJ, bDd, eM, nB, vA, cj, m, asW, aAz, n, fr, LI, aCi, azj, amJ, Nn, cwC, h, dL, Gc, dx, cC, cml, aua, aq, kp, alf, fq, op, iO, asl, dB, hk, Dp, YH, bkE, B, AN, ek, ZZ, AS, yk, fZ, aA, hz, Qo, AO, q, er, eA, eJ, XX, nv, Qg, afQ, aT, aAn, Nc, Fx, jf, iF, s, Ar, Y, ayU, r, en, t, Px, f, ik, ck, sS, nM, auo, cwA, hS, kh, id, Dd, dG, cwy, ad, dP, hd, Db, yF, Yj, d, ff, Xj, aF, bF, es, Lj, cwD, adg, ZW, jr, bsJ); #c1(PTCH1) c2(XP_011517170) c3(protein patched homolog 1 isoform X2) c4(310708) c5(310709, 310710, 310711, 310712, 310713, 310714) c6( ) c7(T, aC, aCh, rV, cwz, fd, ci, aon, xY, bLg, Na, Vp, bv, xy, aE, iv, K, eL, aa, aCg, aW, Az, CT, cwB, Qm, g, cm, aAW, qJ, bDd, eM, nB, vA, cj, m, asW, aAz, n, fr, LI, aCi, azj, amJ, Nn, cwC, h, dL, Gc, dx, cC, cml, aua, aq, kp, alf, fq, op, iO, asl, dB, hk, Dp, YH, bkE, B, AN, ek, ZZ, AS, yk, fZ, aA, hz, Qo, AO, q, er, eA, BJ, XX, nv, Qg, afQ, aT, aAn, Nc, Fx, jf, iF, s, Or, Y, ayU, r, en, t, Px, f, ik, ck, sS, nM, auo, cwA, hS, kh, id, Dd, dG, cwy, ad, dP, hd, Db, yF, Yj, d, ff, Xj, aF, bF, es, Lj, cwD, adg, ZW, jr, bsJ); #c1(PTCH1) c2(XP_011517174) c3(protein patched homolog 1 isoform X1) c4( ) c5(310715, 310716, 310717, 310718, 310719, 310720, 310721, 310722, 310723, 310724, 310725) c6( ) c7(T, aC, aCh, rV, cwz, fd, ci, aon, xY, bLg, Na, Vp, bv, xy, aE, iv, K, eL, aa, aCg, aW, Az, CT, cwB, Qm, g, cm, aAW, qJ, bDd, eM, nB, vA, cj, m, asW, aAz, n, fr, LI, aCi, azj, amJ, Nn, cwC, h, dL, Gc, dx, cC, cml, aua, aq, kp, alf, fq, op, iO, asl, dB, hk, Dp, YH, bkE, B, AN, ek, ZZ, AS, yk, fZ, aA, hz, Do, AD, q, er, eA, eJ, XX, nv, Qg, afQ, aT, aAn, Nc, Fx, jf, iF, s, Ar, Y, ayU, r, en, t, Px, f, iK, ck, sS, nM, auo, cwA, hS, kh, id, Dd, dG, cwy, ad, dP, hd, Db, yF, Yj, d, ff, Xj, aF, bF, es, Lj, cwD, adg, ZW, jr, bsJ); #c1(PTCH1) c2(XP_011517175) c3 (protein patched homolog 1 isoform X3) c4( ) c5(310726, 310727, 310728, 310729, 310730, 310731, 310732) c6( ) c7(T, aC, aCh, rV, cwz, fd, ci, aon, xY, bLg, Na, Vp, bv, xy, aE, iv, K, eL, aa, aCg, aW, Az, CT, cwB, Qm, g, cm, aAW, qJ, bDd, eM, nB, vA, cj, m, asW, aAz, n, fr, LI, aCi, azj, amJ, Nn, cwC, h, dL, Gc, dx, cC, cml, aua, aq, kp, alf, fq, op, iO, asl, dB, hk, Dp, YH, bkE, B, AN, ek, ZZ, AS, yk, fZ, aA, hz, lo, AO, q, er, eA, eJ, XX, nv, Qg, afQ, aT, aAn, Nc, Fx, jf, iF, s, Dr, Y, ayU, r, en, t, Px, f, iK, ck, sS, nM, auo, cwA, hS, kh, id, Dd, dG, cwy, ad, dP, hd, Db, yF, Yj, d, ff, Xj, aF, bF, es, Lj, cwD, adg, ZW, jr, bsJ); #c1(PTCH1) c2(XP_011517176) c3(protein patched homolog 1 isoform X4) c4(310733, 310734, 310735) c5(310736, 310737, 310738, 310739, 310740, 310741, 310742, 310743, 310744, 310745, 310746, 310747, 310748, 310749, 310750, 310751, 310752, 310753, 310754, 310755, 310756, 310757, 310758, 310759, 310760, 310761, 310762, 310763, 310764, 310765, 310766) c6( ) c7(T, aC, aCh, rV, cwz, fd, ci, aon, xY, bLg, Na, Vp, bv, xy, aE, iv, K, eL, aa, aCg, aW, Az, CT, cwB, Am, g, cm, aAW, qJ, bDd, eM, nB, vA, cj, m, asW, aAz, n, fr, LI, aCi, azj, amJ, Nn, cwC, h, dL, Gc, dx, cC, cml, aua, aq, kp, alf, fq, op, iO, asl, dB, hk, Dp, YH, bkE, B, AN, ek, ZZ, AS, yk, fZ, aA, hz, Do, AO, q, er, eA, eJ, XX, nv, Qg, afd, aT, aAn, Nc, Fx, jf, iF, s, Or, Y, ayU, r, en, t, Px, f, ik, ck, sS, nM, auo, cwA, hS, kh, id, Dd, dG, cwy, ad, dP, hd, Db, yF, Yj, d, ff, Xj, aF, bF, es, Lj, cwD, adg, ZW, jr, bsJ); #c1(PTCHD1) c2(NP_775766) c3(patched domain-containing protein 1) c4(250229) c5(250230, 250231, 250232, 250233, 250234, 250235) c6( ) c7(nf, bN, VX, nA, rz); #c1(PTCHD4) c2(NP_001013754) c3(patched domain-containing protein 4 isoform 1) c4(25744) c5(25745, 25746, 25747, 25748, 25749, 25750) c6( ) c7(Ea); #c1(PTCHD4) c2(NP_997382) c3(patched domain-containing protein 4 isoform 2) c4(265486, 265487) c5(265488, 265489, 265490, 265491, 265492, 265493, 265494, 265495, 265496, 265497, 265498, 265499) c6( ) c7(Ea); #c1(PTCRA) c2(NP_001230098) c3(pre T-cell antigen receptor alpha isoform 3 precursor) c4(85900) c5(85901, 85902, 85903, 85904, 85905, 85906) c6( ) c7(kl, A, fR, sQ, apk, kH, uo, ut, dX, lQ, wA, ae); #c1(PTCRA) c2(NP_001230099) c3(pre T-cell antigen receptor alpha isoform 4 precursor) c4(85907) c5(85908, 85909, 85910, 85911, 85912, 85913, 85914, 85915, 85916, 85917, 85918, 85919) c6( ) c7(kl, A, fR, sQ, apK, kH, uo, ut, dX, lQ, wA, ae); #c1(PTCRA) c2(NP_612153) c3(pre T-cell antigen receptor alpha isoform 2 precursor) c4(236100) c5(236101, 236102, 236103, 236104, 236105, 236106) c6( ) c7(kl, A, fR, sl, apk, kH, uo, ut, dX, lQ, wA, ae); #c1(PTCRA) c2(XP_011512618) c3(pre T-cell antigen receptor alpha isoform X1) c4(303585) c5( ) c6( ) c7(kl, A, fR, sQ, apk, kH, uo, ut, dX, lQ, wA, ae); #c1(PTDSS1) c2(NP_001277154) c3(phosphatidylserine synthase 1 isoform 2) c4( ) c5(109173, 109174, 109175, 109176, 109177, 109178) c6( ) c7(cwE, aq, V, B, hD); #c1(PTDSS1) c2(NP_055569) c3 (phosphatidylserine synthase 1 isoform 1) c4(181982, 181983) c5(181984, 181985, 181986, 181987, 181988, 181989, 181990) c6( ) c7(cwE, aq, V, B, hD); #c1(PTEN) c2(NP_000305) c3(phosphatidylinositol 3,4,5-trisphosphate 3-phosphatase and dual-specificity protein phosphatase PTEN isoform PTEN) c4( ) c5(6766, 6767, 6768, 6769, 6770) c6( ) c7(gh, T, fd, ci, Ks, bv, aE, awe, ajv, cwN, dV, HR, hu, cp, aa, ZH, iE, wp, Ls, Az, CT, vs, PQ, zE, ahv, iA, nB, cj, cX, k, ht, DE, cwf, aj, auA, PR, cd, cwM, lG, ey, aQM, ox, on, pl, YI, MD, ahJ, dB, eD, zG, qF, aMI, cwJ, AN, OD, aA, cz, lu, AO, q, PW, qK, cy, rO, oF, rz, lM, amw, cwK, bf, NE, eS, cl, cwl, iM, RW, bt, On, qz, uG, atS, jh, aHv, KO, aue, ad, afb, hX, iC, ccJ, yt, oW, bj, lQ, wm, BO, Ub, ff, dq, Sm, bz, rZ, amS, ag, i, aed, iu, ne, jr, bM, cwO, bkF, if, bB, aon, ip, x, aeO, aJu, xQ, eG, JI, fc, hl, fH, Qq, Qm, as, eF, cm, aHd, qJ, bkD, go, vA, mx, Ql, ik, wu, Pi, n, PY, nA, Cd, b, jz, pe, oC, gd, AD, bP, GU, hL, hj, at, B, ul, AS, dA, cwP, lH, v, Yn, hW, BJ, aHs, ae, jf, Qr, vT, ev, Lf, aZg, aom, bu, t, apx, ck, kP, cwH, gX, anD, oX, hl, aBz, Fc, hd, aop, py, Db, cwG, nJ, mu, d, je, aiz, adj, dr, bF, D, afd, y, aC, hV, xE, Bc, Qk, M, K, eL, bWL, Ku, g, iP, AM, QF, m, cwl, Le, zX, aw, NT, qy, ft, kH, KV, CN, cM, ML, XT, Ld, kT, aKA, zL, Az, lB, it, fM, hz, eA, J, er, XX, lg, hb, qv, xR, Y, r, en, ik, bN, Dy, auo, np, LS, id, aZ, iJ, Jm, dP, fX, hP, cfu, Yi, Gv, xs, eH, bow, iR, Nq, beC, pu, Ew, cP, ix, cwl, Tb, ahc, apr, bZe, aR, il, Fo, MO, aAu, aW, eM, iT, gn, aEj, akt, fr, qN, ai, BI, hw, kl, h, jy, dL, aDC, he, Xh, OV, asp, aq, kp, mo, op, oU, hk, Vy, hR, HB, oh, cn, bL, yk, cR, yO, bBs, mj, cf, aoA, bUn, aT, Fx, Tj, an, qw, s, iU, oV, nM, adR, aJQ, VB, hS, ow, ha, Fb, OH, av, Rq, NS, fb, pq, iL, eZ, Ej, aF, Hp, LB, es, eL, bE, aS, MC, X, bND); #c1(PTEN) c2(NP_001291646) c3(phosphatidylinositol 3,4,5-trisphosphate 3-phosphatase and dual-specificity protein phosphatase PTEN isoform PTEN-L) c4(116298) c5(116299) c6( ) c7(gh, T, fd, ci, Ks, bv, aE, awe, ajv, cwN, dV, HR, hu, cp, aa, ZH, iE, wp, Ls, Az, CT, vs, PQ, ZE, ahv, iA, nB, cj, cX, k, ht, DE, cwf, aj, auA, PR, cd, cwM, lG, ey, aQM, ox, on, pl, YI, MD, ahJ, dB, eD, zG, qF, aMl, cwJ, AN, OD, aA, cz, lu, AD, q, PW, qK, cy, rO, oF, rz, lM, amw, cwK, bf, NE, eS, cl, cwl, iM, RW, bt, On, qz, uG, atS, jh, aHv, KO, aue, ad, afb, hX, iC, ccJ, yt, oW, bj, lQ, wm, BO, Ub, ff, dq, Sm, bz, rZ, amS, ag, i, aed, iu, ne, jr, bM, cwO, bkF, if, bB, aon, ip, x, aeO, aJu, xQ, eG, JI, fc, hl, fH, Qq, Qm, as, eF, cm, aHd, qJ, bkD, go, vA, mx, Ql, ik, wu, Pi, n, PY, nA, Cd, b, jz, pe, oC, gd, AQ, bP, GU, hL, hj, at, B, ul, AS, dA, cwP, lH, v, Yn, hW, BJ, aHs, ae, jf, Or, vT, ev, Lf, aZg, aom, bu, t, apx, ck, kP, cwH, gX, anD, oX, hl, aBz, Fc, hd, aop, py, Db, cwG, nJ, mu, d, je, aiz, adj, dr, bF, D, afd, y, aC, hV, xE, Bc, Qk, M, K, eL, bWL, Ku, g, iP, AM, QF, m, cwl, Le, zX, aw, NT, qy, ft, KH, KV, CN, CM, ML, XT, Ld, kT, aKA, zl, Qz, lB, it, fM, hz, eA, J, er, XX, lg, hb, qv, xR, Y, r, en, ik, bN, Dy, auo, np, LS, id, aZ, iJ, Jm, dP, fX, hP, cfu, Yi, Gv, xs, eH, bow, iR, Nq, beC, pu, Ew, cP, ix, cwl, Tb, ahc, apr, bZe, aR, il, Fo, MO, aAu, aW, eM, iT, gn, aEj, akt, fr, qN, ai, BI, hw, kl, h, jy, dL, aDC, he, Xh, OV, asp, aq, kp, mo, op, oU, hk, Vy, hR, HB, oh, cn, bl, yk, cR, yO, bBs, mj, cF, aoA, bUn, aT, Fx, Tj, an, qw, s, iU, oV, nM, adR, aJQ, VB, hS, ow, ha, Fb, OH, av, Rq, NS, fb, pq, iL, eZ, Ej, aF, Hp, LB, es, eL, bE, aS, MC, X, bND); #c1(PTER) c2(NP_001248766) c3(phosphotriesterase-related protein isoform 2) c4(94017) c5(94018, 94019, 94020, 94021, 94022, 94023) c6( ) c7(bL, eR, Y, dk, hl, r, aq, v, aY, q); #c1(PTER) c2(NP_109589) c3(phosphotriesterase-related protein isoform 1) c4(219661) c5(219662, 219663, 219664, 219665, 219666, 219667, 219668, 219669, 219670, 219671, 219672, 219673, 219674, 219675, 219676, 219677, 219678, 219679) c6( ) c7(bL, eR, Y, dk, hl, r, aq, v, aY, q); #c1(PTF1A) c2(NP_835455) c3(pancreas transcription factor 1 subunit alpha) c4(253505) c5(253506, 253507, 253508, 253509, 253510, 253511, 253512) c6( ) c7(Ng, axl, mh, aE, cwR, cpC, atf, kp, eJ); #c1(PTGDR) c2(XP_005267948) c3(prostaglandin D2 receptor isoform X1) c4(281327) c5(281328, 281329, 281330, 281331, 281332, 281333) c6( ) c7(bF, bM, av, af, z, sp, cwS, ff, jh, wb, m, en, eE, cm, B, ek, L); #c1(PTGDR2) c2(NP_004769) c3(prostaglandin D2 receptor 2) c4(149445) c5(149446, 149447, 149448, 149449, 149450, 149451) c6( ) c7(bM, aDy, aD, jh, n, Jx, q, XI, tN, Nr, sp, r, po, eE, ve, DF, Nq, cT, z, Hb, Tu, fD); #c1(PTGDS) c2(NP_000945) c3(prostaglandin-H2 D-isomerase precursor) c4(17585, 17586, 17587, 17588, 17589) c5(17590, 17591, 17592, 17593, 17594, 17595, 17596, 17597, 17598, 17599, 17600, 17601, 17602, 17603, 17604, 17605, 17606, 17607, 17608, 17609, 17610, 17611, 17612, 17613, 17614, 17615, 17616, 17617, 17618, 17619, 17620, 17621, 17622, 17623, 17624, 17625, 17626, 17627, 17628, 17629, 17630, 17631, 17632, 17633, 17634, 17635) c6( ) c7(bL, bM, T, dD, akd, eR, aDy, ea, aD, rl, qy, bLf, hz, aj, bB, OY, ci, eq, jh, q, J, ad, ip, Vp, b, hd, ON, aMT, aT, hD, jy, bK, K, ji, ae, af, yF, De, jf, OV, d, yi, aW, sp, Y, az, cr, r, rM, cwU, dN, bBH, VI, Au, as, iV, eE, t, f, ale, zV, Ew, nB, dB, bE, asl, mG, z, cwT, uz, cS, m, Hb, fD, i, Bh, L); #c1(PTGER1) c2(NP_000946) c3(prostaglandin E2 receptor EPI subtype) c4(17636) c5(17637, 17638, 17639, 17640, 17641, 17642) c6( ) c7(bM, sY, kh, aqo, cz, bB, zP, av, di, af, zQ, qt, ff, en, g, uC, ek, ac, bE, dJ, m, B); #c1(PTGER2) c2(NP_000947) c3(prostaglandin E2 receptor EP2 subtype) c4(17643) c5(17644, 17645, 17646, 17647, 17648, 17649) c6( ) c7(bM, sY, T, fR, sQ, pf, e, aw, NT, aA, j, ci, q, ad, EI, eW, dP, av, M, aT, lQ, bau, ae, af, s, cwV, Rr, r, aF, g, t, uC, cm, kT, cwW, c, eM, afc, wB, uo, ut, Tu, y); #c1(PTGER3) c2(XP_011540 μl) c3(prostaglandin E2 receptor EP3 subtype isoform X1) c4(338466) c5(338467, 338468, 338469, 338470, 338471, 338472) c6( ) c7(bM, sY, T, xS, fR, s, aA, bB, ad, fS, K, lQ, aNR, ae, af, DA, kl, aRL, aq, aF, g, as, iR, uC, bF, bE, uo, ut, m); #c1(PTGER3) c2(XP_011540112) c3 (prostaglandin E2 receptor EP3 subtype isoform X2) c4(338473) c5(338474) c6( ) c7(bM, sY, T, xS, fR, sQ, aA, bB, ad, fS, K, lQ, aNR, ae, af, DA, kl, aRL, aq, aF, g, as, iR, uC, bF, bE, uo, ut, m); #c1(PTGER4) c2(NP_000949) c3(prostaglandin E2 receptor EP4 subtype) c4(17650) c5(17651, 17652, 17653, 17654, 17655, 17656) c6( ) c7(bL, bM, sY, T, fM, pG, xb, DD, dD, pf, if, NT, jh, aA, cz, bB, j, ci, eq, qN, fr, V, q, ad, eW, dP, av, aE, M, al, ae, af, tN, A, mR, bf, s, aW, sp, fO, Rr, r, fe, Au, aF, g, iV, cm, aTQ, uC, ck, gt, eM, xZ, bE, AM, NI, EY, aQY, qR, B, y); #c1(PTGES) c2(NP_004869) c3(prostaglandin E synthase) c4(150446) c5(150447, 150448, 150449, 150450, 150451) c6( ) c7(T, fM, dA, fR, sQ, id, pf, atC, atj, ht, aw, ul, aA, cz, bB, awr, jh, ci, q, ad, eA, dP, av, jz, xE, dY, lg, aT, lG, cy, K, lQ, ae, af, eu, DI, bf, s, JI, aW, ff, fO, LZ, r, fe, hN, aF, en, g, as, t, zE, iK, ac, c, eM, xZ, bE, U, uo, uz, ut, hE, it, B, y); #c1(PTGES) c2(XP_011517512) c3(prostaglandin E synthase isoform X1) c4( ) c5(311306) c6( ) c7(T, fM, dA, fR, s, id, pf, atC, atj, ht, aw, ul, aA, cz, bB, awr, jh, ci, q, ad, eA, dP, av, jz, xE, dY, lg, aT, lG, cy, K, lQ, ae, af, eu, DI, bf, s, Jl, aW, ff, fO, LZ, r, fe, hN, aF, en, g, as, t, zE, ik, ac, c, eM, xZ, bE, U, uo, uz, ut, hE, it, B, y); #c1(PTGES2) c2(NP_079348) c3(prostaglandin E synthase 2 isoform 1) c4(217784, 217785) c5(217786, 217787, 217788, 217789, 217790, 217791, 217792, 217793) c6( ) c7(bL, T, af, nB, jx, r, uz, awr, mf, v, m, ei, g, pd, ae, q, ad); #c1(PTGES2) c2(XP_011517353) c3 (prostaglandin E synthase 2 isoform X1) c4( ) c5(311084, 311085, 311086, 311087, 311088) c6( ) c7(bL, T, af, nB, jx, r, uz, awr, mf, v, m, ei, g, pd, ae, q, ad); #c1(PTGES3) c2(NP_006592) c3(prostaglandin E synthase 3 isoform a) c4(166498) c5(166499, 166500, 166501, 166502, 166503, 166504) c6( ) c7(bM, T, eG, W, bP, fO, Pg, U, aT, aA, uz, auT, aF, aU, K, g, alR, alP, ae, ad); #c1(PTGIR) c2(NP_000951) c3(prostacyclin receptor) c4(17657) c5(17658, 17659, 17660, 17661, 17662, 17663) c6( ) c7(P, bM, dD, ea, e, gZ, Au, aS, Tu, eq, af); #c1(PTGIS) c2(NP_000952) c3(prostacyclin synthase precursor) c4(17664) c5(17665, 17666, 17667, 17668, 17669, 17670) c6( ) c7(ID, Dw, dD, eR, ea, e, al, aw, cz, bB, aj, eq, fu, bX, q, J, av, afm, aT, cy, af, eu, wC, ly, mR, ue, s, aW, ff, r, Au, zC, en, g, bz, qa, lx, ek, nM, ac, eM, bE, m, ei, lC); #c1(PTGR1) c2(NP_001139581) c3(prostaglandin reductase 1 isoform 2) c4(59529, 59530) c5(59531, 59532, 59533, 59534, 59535, 59536, 59537, 59538) c6( ) c7(aq, bz, B, g, hS); #c1(PTGR1) c2(NP_036344) c3(prostaglandin reductase 1 isoform 1) c4(173091, 173092) c5(173093, 173094, 173095, 173096, 173097, 173098, 173099, 173100, 173101, 173102, 173103) c6( ) c7(aq, bz, B, g, hS); #c1(PTGR1) c2(XP_011516696) c3(prostaglandin reductase 1 isoform X1) c4(310103) c5(310104, 310105, 310106, 310107, 310108, 310109, 310110, 310111, 310112, 310113, 310114, 310115, 310116, 310117, 310118, 310119, 310120, 310121, 310122) c6( ) c7(aq, bz, B, g, hS); #c1(PTGR1) c2(XP_011516697) c3(prostaglandin reductase 1 isoform X2) c4( ) c5(310123, 310124, 310125, 310126, 310127, 310128, 310129, 310130) c6( ) c7(aq, bz, B, g, hS); #c1(PTGS1) c2(NP_000953) c3(prostaglandin G/H synthase 1 isoform 1 precursor) c4(17671) c5(17672, 17673, 17674, 17675, 17676, 17677, 17678) c6( ) c7(cwY, bM, T, fR, pf, ea, if, e, eb, bB, amj, fd, ci, afr, aE, rw, fS, M, K, de, eL, aa, Mq, aW, ado, az, aNq, aB, g, sz, uC, aWd, fT, jV, eM, iT, wB, uo, su, m, dD, sl, aw, ht, NT, n, aj, fr, ct, qN, cd, ai, cqH, Yc, lG, dL, pl, OV, sp, lb, fe, cM, SE, at, ut, acJ, fl, eR, aA, cz, hy, j, bX, q, J, nv, qp, aT, Di, wP, jj, bq, ae, af, dL, eu, ajU, s, iU, fO, afS, Pg, r, akY, lo, aPf, en, apx, t, iK, dS, ck, sS, tW, ac, gX, fD, ao, On, Kd, sY, BD, akd, MH, aD, iW, bS, eq, mC, ad, wD, av, ds, cwX, lQ, vP, d, ff, eZ, iV, Z, bz, LB, bF, wQ, Bv, zr, bE, ei, MC, lC, X, y); #c1(PTGS1) c2(NP_001258093) c3 (prostaglandin G/H synthase 1 isoform 3 precursor) c4(95957) c5(95958, 95959, 95960, 95961, 95962, 95963) c6( ) c7(cwY, bM, T, FR, pf, ea, if, e, eb, bB, amj, fd, ci, afr, aE, rw, fS, M, K, de, eL, aa, Mq, aW, aoo, az, aNq, aB, g, sz, uC, aWd, fT, jV, eM, iT, wB, uo, su, m, dD, sQ, aw, ht, NT, n, aj, fr, ct, qN, cd, ai, cqH, Yc, lG, dL, pl, OV, sp, lb, fe, cM, SE, at, ut, acJ, fl, eR, aA, cz, hy, j, bX, q, J, nv, qp, aT, Di, wP, j, bq, ae, af, dL, eu, ajU, s, iU, fO, afS, Pg, r, akY, lo, aPf, en, apx, t, ik, dS, ck, sS, tW, ac, gX, fD, ao, On, Kd, sY, BD, akd, MH, aD, iW, bS, eq, mC, ad, wD, av, ds, cwX, lQ, vP, d, ff, eZ, iV, Z, bz, LB, bF, wQ, Bv, zr, bE, ei, MC, lC, X, y); #c1(PTGS1) c2(NP_001258297) c3(prostaglandin G/H synthase 1 isoform 6) c4(96011) c5(96012, 96013, 96014, 96015, 96016, 96017) c6( ) c7(cwY, bM, T, fR, pf, ea, if, e, eb, bB, amj, fd, ci, afr, aE, rw, fS, M, K, dQ, eL, aa, Mq, aW, aoo, az, aNq, aB, g, sz, uC, aWd, fT, jV, eM, iT, wB, uo, su, m, dD, se, aw, ht, NT, n, aj, fr, ct, qN, cd, ai, cqH, Yc, lG, dL, pl, OV, sp, Ib, fe, cM, SE, at, ut, acJ, fl, eR, aA, cz, hy, j, bX, q, J, nv, qp, aT, Di, wP, j, bq, ae, af, dL, eu, ajU, s, iU, fO, afS, Pg, r, akY, lo, aPf, en, apx, t, ik, dS, ck, sS, tW, ac, gX, fD, ao, On, Kd, sY, BD, akd, MH, aD, iW, bS, eq, mC, ad, wD, av, ds, cwX, lQ, vP, d, ff, eZ, iV, Z, bz, LB, bF, wL, Bv, zr, bE, ei, MC, lC, X, y); #c1(PTGS1) c2(NP_542158) c3(prostaglandin G/H synthase 1 isoform 2 precursor) c4(231452) c5(231453, 231454, 231455, 231456, 231457, 231458) c6( ) c7(cwY, bM, T, fR, pf, ea, if, e, eb, bB, amj, fd, ci, afr, aE, rw, fS, M, K, de, eL, aa, Mq, aW, aoo, az, aNq, aB, g, sz, uC, aWd, fT, jV, eM, iT, wB, uo, sU, m, dD, sQ, aw, ht, NT, n, aj, fr, ct, qN, cd, ai, cqH, Yc, lG, dL, pl, OV, sp, lb, fe, cM, SE, at, ut, acJ, fl, eR, aA, cz, hy, j, bX, q, J, nv, qp, aT, Di, wP, j, bq, ae, af, dL, eu, ajU, s, iU, fO, afS, Pg, r, akY, lo, aPf, en, apx, t, ik, dS, ck, sS, tW, ac, gX, fD, ao, On, Kd, sY, BD, akd, MH, aD, iW, bS, eq, mC, ad, wD, av, ds, cwX, lQ, vP, d, ff, eZ, iV, Z, bz, LB, bF, wL, Bv, zr, bE, ei, MC, lC, X, y); #c1(PTGS1) c2(XP_011517178) c3(prostaglandin G/H synthase 1 isoform X1) c4(310767) c5(310768, 310769, 310770, 310771, 310772) c6( ) c7(cwY, bM, T, FR, pf, ea, if, e, eb, bB, amj, fd, ci, afr, aE, rw, fS, M, K, dQ, eL, aa, Mq, aW, aoo, az, aNq, aB, g, sz, uC, aWd, fT, jV, eM, iT, wB, uo, sU, m, dD, sQ, aw, ht, NT, n, aj, fr, ct, qN, cd, ai, cqH, Yc, lG, dL, pl, OV, sp, lb, fe, cM, SE, at, ut, acJ, fl, eR, aA, cz, hy, j, bX, q, J, nv, qp, aT, Di, wP, j, bq, ae, af, dL, eu, ajU, s, iU, fO, afS, Pg, r, akY, lo, aPf, en, apx, t, ik, dS, ck, sS, tW, ac, gX, fD, ao, On, Kd, sY, BD, akd, MH, aD, iW, bS, eq, mC, ad, wD, av, ds, cwX, lQ, vP, d, ff, eZ, iV, Z, bz, LB, bF, wL, Bv, zr, bE, ei, MC, lC, X, y); #c1(PTGS2) c2(NP_000954) c3 (prostaglandin G/H synthase 2 precursor) c4(17679) c5(17680, 17681, 17682, 17683, 17684, 17685) c6( ) c7(uB, gh, T, ea, bO, fd, ci, bv, aE, rw, dV, Ds, HR, hu, cp, aa, li, az, nY, vs, axp, zV, ba, fT, jV, gt, nB, cj, k, eg, cE, aNm, dt, Uc, ht, RC, aj, cd, lG, dd, pl, MD, fQ, fe, hq, dB, ajd, E, qF, aZD, AN, aJC, DD, fZ, aA, cz, wy, lu, AD, q, Di, cy, j, dL, eu, bf, fO, bg, lo, cl, pb, rR, ac, bt, rA, ao, On, BD, iW, uG, rl, jh, KO, dG, uu, fu, ad, afb, hX, iC, wD, cwX, lQ, vP, WZ, DI, BO, f, gC, aEW, Sm, iV, bz, DF, DT, xZ, lC, ne, iu, oO, bM, if, e, bB, Tz, ip, Yy, azm, bc, akl, De, xQ, dH, JI, Oj, fc, hl, kt, as, ir, eE, eF, cm, Qt, aWd, qJ, vA, vy, hh, in, dD, cwZ, n, jz, gd, eV, hn, A, RT, PX, lb, po, yq, bP, dC, PI, bQU, hL, at, ut, fl, sa, B, B, AS, lH, eX, v, Qo, V, eJ, cqV, bGn, hW, wN, ld, avt, bau, ae, jf, Yw, zH, ev, Lf, acB, bu, apx, LV, t, ck, wL, gX, Tu, vi, nZ, MH, OR, eq, Fc, hd, Db, ds, Hk, LA, sj, Jc, Yj, d, NH, rM, th, Z, bF, D, gk, Hg, y, pC, aC, UH, FR, hV, atj, al, qj, lR, afr, u, Bc, M, K, GO, wt, eL, avn, qS, aoo, ml, LZ, hN, aB, g, uC, ou, AM, nF, U, uo, m, sl, sw, zX, aw, NT, BH, BF, zP, ft, pN, lt, cU, wb, KV, ks, agF, jT, cM, ar, afc, Pr, it, L, fM, fG, jx, pQ, hz, j, acn, il, pd, eA, J, er, akE, XX, lg, yg, bq, xR, Y, aZs, Pg, r, aou, en, iK, bN, Dy, c, dh, dy, EY, fD, GI, sY, kh, id, akd, bkL, uO, aET, bS, ah, aAq, dP, oj, io, vB, ji, NM, WW, lU, BN, lR, vj, eH, iR, cuy, Nq, Bv, Lj, ei, hE, pf, fV, eb, crk, aR, dY, aDk, H, DA, ayT, aAu, aW, eM, iT, wB, aep, uJ, QS, cZ, aEj, aBx, awr, fr, ct, qN, KO, ai, eW, co, BI, cc, hw, Yc, h, aPe, dL, hB, db, Nr, aq, Hu, op, hk, FV, bm, hR, uz, acJ, cir, bL, Dw, lk, eR, yk, cR, hy, C, akc, ga, TI, mP, uT, nv, LY, qp, afQ, aT, UN, zg, an, af, ak, s, iU, oV, akY, f, dS, sS, nM, pZ, alt, AH, aDj, VB, hS, Kd, aD, bmJ, Fb, OH, jE, mC, av, l, F, ib, oz, NS, Yo, jo, ul, qt, eZ, asV, Ej, aF, aDd, S, ek, LB, es, aqM, bE, uR, aS, tr, MC, adg, X, Ex); #c1(PTH) c2(NP_000306) c3(parathyroid hormone preproprotein) c4(6771) c5(6772, 6773, 6774, 6775, 6776, 6777, 6778, 6779, 6780, 6781, 6782, 6783) c6(6784, 6785, 6786, 6787) c7(T, rV, ea, e, al, bBV, ci, aTS, cxa, aY, cpi, aJO, acX, bsH, bv, aE, pX, ari, K, EL, dQ, azh, bDT, KF, aW, Np, Lm, eF, Vm, nB, cxd, nF, TG, iS, QF, aYS, k, azg, lS, Ly, aj, nA, ml, JW, cd, adf, amB, ft, zw, kH, jy, eV, dL, bta, aAG, OM, he, hi, A, aAl, OX, Hz, arp, aq, aLi, YE, ln, FV, hj, tl, at, pg, aAQ, B, vQ, bL, AB, eR, ex, cz, cR, adi, wo, amC, j, avO, pd, J, er, bke, xx, ahy, TI, nv, xW, aT, yu, rD, zg, zp, rz, ae, af, aeY, bA, kl, s, iU, oV, AA, EJ, bRK, Qi, arr, rR, acW, pa, yl, iM, cxh, hr, fP, agf, dy, bt, aAO, ars, cxg, pY, aZ, ha, jN, Ay, cxc, xt, ad, fU, oW, bsR, yh, Yi, aAL, Jg, je, vj, eH, OQ, cxb, nh, cxe, dq, HI, bz, dr, wW, qa, pv, gk, aMa, cxf, yE, bE, bon, X); #c1(PTH1R) c2(NP_000307) c3(parathyroid hormone/parathyroid hormone-related peptide receptor precursor) c4(6788, 6789, 6790) c5(6791, 6792, 6793, 6794, 6795, 6796, 6797, 6798, 6799, 6800, 6801, 6802, 6803, 6804, 6805, 6806, 6807, 6808, 6809, 6810, 6811, 6812, 6813, 6814, 6815, 6816, 6817, 6818, 6819, 6820) c6( ) c7(T, kh, aC, eR, fR, wo, fr, ci, cxa, aY, ahy, xW, aT, af, azh, bA, aa, hi, kF, aW, vj, eH, cxj, eF, dr, nB, cxk, cxi, ln, azg, k, qF, cxl, i, X, ars); #c1(PTH2) c2(NP_848544) c3(tuberoinfundibular peptide of 39 residues preproprotein) c4( ) c5(253806, 253807, 253808, 253809, 253810, 253811) c6(253812) c7(ft); #c1(PTH2) c2(XP_011524711) c3(tuberoinfundibular peptide of 39 residues isoform X1) c4(319206) c5(319207) c6( ) c7(ft); #c1(PTH2R) c2(NP_005039) c3(parathyroid hormone 2 receptor precursor) c4(152374) c5(152375, 152376, 152377, 152378, 152379, 152380) c6( ) c7(bA, aDR, ac, aDQ); #c1(PTHLH) c2(NP_945315) c3(parathyroid hormone-related protein isoform 2 preproprotein) c4(260767, 260768, 260769) c5(260770, 260771, 260772, 260773, 260774, 260775, 260776, 260777, 260778, 260779, 260780, 260781, 260782, 260783, 260784, 260785, 260786, 260787, 260788, 260789, 260790, 260791, 260792, 260793, 260794, 260795, 260796, 260797, 260798, 260799) c6( ) c7(T, aC, Tb, fR, ea, eb, bB, fd, zh, ci, cxa, mF, aY, aR, ip, bv, aE, Xc, pX, Eo, cxo, rB, K, EL, azh, aa, kF, aW, Np, cxm, cxn, g, as, eF, cm, cpE, eM, iA, avZ, iT, wE, vA, iS, azg, m, iu, cZ, dD, zt, aj, fr, cd, ke, ft, zw, KH, Ne, yZ, wb, ey, dd, op, asE, ar, KE, YE, Qu, ln, tl, pg, qF, bee, B, um, cn, eR, lH, sM, GC, lu, q, J, eA, eJ, mj, hb, xW, rS, yg, aT, rD, zp, aOV, ae, aeY, bA, s, vT, iU, Y, ev, r, lo, en, t, EJ, bRK, ck, iM, TJ, aHB, cxh, agf, Da, xb, VB, cxg, kh, dM, jN, jh, Hn, Fb, eq, iG, xt, ad, av, Tg, py, cxp, cb, Yi, ul, je, qt, ff, eH, HI, bz, dr, es, yE, bE, i, hE, X, y); #c1(PTHLH) c2(XP_011519077) c3(parathyroid hormone-related protein isoform X1) c4(313672) c5(313673, 313674, 313675, 313676, 313677, 313678, 313679, 313680, 313681, 313682, 313683, 313684, 313685, 313686) c6( ) c7(T, aC, Tb, FR, ea, eb, bB, fd, zh, ci, cxa, mF, aY, aR, ip, bv, aE, Xc, pX, Eo, cxo, rB, K, EL, azh, aa, kF, aW, Np, cxm, cxn, g, as, eF, cm, cpE, eM, iA, avZ, iT, wE, vA, iS, azg, m, iu, cZ, dD, zt, aj, fr, cd, ke, ft, zw, kH, Ne, yZ, wb, ey, dd, op, asE, ar, KE, YE, Qu, ln, tl, pg, qF, bee, B, um, cn, eR, lH, sM, GC, lu, q, J, eA, eJ, mj, hb, xW, rS, yg, aT, rD, zp, aOV, ae, aeY, bA, s, vT, iU, Y, ev, r, lo, en, t, EJ, bRK, ck, iM, TJ, aHB, cxh, agf, Da, xb, VB, cxg, kh, dM, jN, jh, Hn, Fb, eq, iG, xt, ad, av, Tg, py, cxp, cb, Yi, ul, je, qt, ff, eH, HI, bz, dr, es, yE, bE, i, HE, X, y); #c1(PTK2) c2(NP_001186578) c3(focal adhesion kinase 1 isoform c) c4(77876, 77877) c5(77878, 77879, 77880, 77881, 77882, 77883, 77884, 77885, 77886, 77887, 77888, 77889) c6( ) c7(T, ht, aA, Bm, n, cz, bB, fr, ci, wo, fd, zP, V, iJ, q, ad, eA, hX, eJ, ip, av, iu, jz, yt, Dr, ds, aT, h, lG, cy, hP, K, NM, ae, dx, hm, aa, d, s, MD, iU, aW, Y, ff, eH, eZ, r, fQ, aq, aF, yq, BG, en, g, as, t, eF, bz, cm, iK, LB, qJ, nB, bE, Qu, dh, X, cj, pg, dy, m, y, B); #c1(PTK2) c2(NP_005598) c3(focal adhesion kinase 1 isoform b) c4(157823, 157824, 157825, 157826, 157827, 157828) c5(157829, 157830, 157831, 157832, 157833, 157834, 157835, 157836, 157837, 157838, 157839, 157840, 157841, 157842, 157843, 157844, 157845, 157846, 157847, 157848, 157849, 157850, 157851, 157852, 157853, 157854, 157855, 157856, 157857, 157858, 157859, 157860, 157861, 157862, 157863, 157864, 157865, 157866) c6( ) c7(T, ht, aA, Bm, n, cz, bB, fr, ci, wo, fd, zP, V, iJ, q, ad, eA, hX, eJ, ip, av, iu, jz, yt, Dr, ds, aT, h, lG, cy, hP, K, NM, ae, dx, hm, aa, d, s, MD, iU, aW, Y, ff, eH, eZ, r, fQ, aq, aF, yq, BG, en, g, as, t, eF, bz, cm, ik, LB, qJ, nB, bE, Qu, dh, X, cj, pg, dy, m, y, B); #c1(PTK2) c2(NP_722560) c3(focal adhesion kinase 1 isoform a) c4(246850, 246851, 246852, 246853) c5(246854, 246855, 246856, 246857, 246858, 246859, 246860, 246861, 246862, 246863, 246864, 246865, 246866, 246867, 246868, 246869, 246870, 246871, 246872, 246873, 246874, 246875, 246876, 246877, 246878, 246879, 246880, 246881, 246882, 246883, 246884, 246885, 246886, 246887) c6( ) c7(T, ht, aA, Bm, n, cz, bB, fr, ci, wo, fd, zP, V, iJ, q, ad, eA, hX, eJ, ip, av, iu, jz, yt, Dr, ds, aT, h, lG, cy, hP, K, NM, ae, dx, hm, aa, d, s, MD, iU, aW, Y, ff, eH, eZ, r, fQ, aq, aF, yq, BG, en, g, as, t, eF, bz, cm, iK, LB, qJ, nB, bE, Qu, dh, X, cj, pg, dy, m, y, B); #c1(PTK2) c2(XP_005251060) c3(focal adhesion kinase 1 isoform X4) c4( ) c5(270737, 270738, 270739, 270740, 270741, 270742, 270743, 270744, 270745, 270746) c6( ) c7(T, ht, aA, Bm, n, cz, bB, fr, ci, wo, fd, zP, V, iJ, q, ad, eA, hX, eJ, ip, av, iu, jz, yt, Dr, ds, aT, h, lG, cy, hP, K, NM, ae, dx, hm, aa, d, s, MD, iU, aW, Y, ff, eH, eZ, r, fQ, aq, aF, yq, BG, en, g, as, t, eF, bz, cm, ik, LB, qJ, nB, bE, Qu, dh, X, cj, pg, dy, m, y, B); #c1(PTK2) c2(XP_006716669) c3(focal adhesion kinase 1 isoform X13) c4(290423) c5(290424, 290425) c6( ) c7(T, ht, aA, Bm, n, cz, bB, fr, ci, wo, fd, zP, V, iJ, q, ad, eA, hX, eJ, ip, av, iu, jz, yt, Dr, ds, aT, h, lG, cy, hP, K, NM, ae, dx, hm, aa, d, s, MD, iU, aW, Y, ff, eH, eZ, r, fQ, aq, af, yq, BG, en, g, as, t, eF, bz, cm, ik, LB, qJ, nB, bE, Qu, dh, X, cj, pg, dy, m, y, B); #c1(PTK2) c2(XP_011515482) c3(focal adhesion kinase 1 isoform X1) c4( ) c5(308293, 308294, 308295, 308296, 308297, 308298, 308299, 308300, 308301, 308302, 308303) c6( ) c7(T, ht, aA, Bm, n, cz, bB, fr, ci, wo, fd, zP, V, iJ, q, ad, eA, hX, eJ, ip, av, iu, jz, yt, Dr, ds, aT, h, lG, cy, hP, K, NM, ae, dx, hm, aa, d, s, MD, iU, aW, Y, ff, eH, eZ, r, fQ, aq, aF, yq, BG, en, g, as, t, eF, bz, cm, ik, LB, qJ, nB, bE, Qu, dh, X, cj, pg, dy, m, y, B); #c1(PTK2) c2(XP_011515484) c3(focal adhesion kinase 1 isoform X3) c4(308304, 308305) c5(308306, 308307, 308308, 308309, 308310, 308311, 308312, 308313, 308314, 308315, 308316, 308317) c6( ) c7(T, ht, aA, Bm, n, cz, bB, fr, ci, wo, fd, zP, V, iJ, q, ad, eA, hX, eJ, ip, av, iu, jz, yt, Dr, ds, aT, h, lG, cy, hP, K, NM, ae, dx, hm, aa, d, s, MD, iU, aW, Y, ff, eH, eZ, r, fQ, aq, aF, yq, BG, en, g, as, t, eF, bz, cm, ik, LB, qJ, nB, bE, Qu, dh, X, cj, pg, dy, m, y, B); #c1(PTK2) c2(XP_011515488) c3(focal adhesion kinase 1 isoform X8) c4(308318) c5( ) c6( ) c7(T, ht, aA, Bm, n, cz, bB, fr, ci, wo, fd, zP, V, iJ, q, ad, eA, hX, eJ, ip, av, iu, jz, yt, Dr, ds, aT, h, lG, cy, hP, K, NM, ae, dx, hm, aa, d, s, MD, iU, aW, Y, ff, eH, eZ, r, fQ, aq, af, yq, BG, en, g, as, t, eF, bz, cm, ik, LB, qJ, nB, bE, Qu, dh, X, cj, pg, dy, m, y, B); #c1(PTK2) c2(XP_011515492) c3(focal adhesion kinase 1 isoform X12) c4(308319, 308320, 308321, 308322) c5(308323, 308324, 308325, 308326, 308327, 308328, 308329, 308330, 308331, 308332, 308333) c6( ) c7(T, ht, aA, Bm, n, cz, bB, fr, ci, wo, fd, zP, V, iJ, q, ad, eA, hX, eJ, ip, av, iu, jz, yt, Dr, ds, aT, h, lG, cy, hP, K, NM, ae, dx, hm, aa, d, s, MD, iU, aW, Y, ff, eH, eZ, r, fQ, aq, aF, yq, BG, en, g, as, t, eF, bz, cm, ik, LB, qJ, nB, bE, Qu, dh, X, cj, pg, dy, m, y, B); #c1(PTK2) c2(XP_011515494) c3(focal adhesion kinase 1 isoform X15) c4(308334) c5(308335) c6( ) c7(T, ht, aA, Bm, n, cz, bB, fr, ci, wo, fd, zP, V, iJ, q, ad, eA, hX, eJ, ip, av, iu, jz, yt, Dr, ds, aT, h, lG, cy, hP, K, NM, ae, dx, hm, aa, d, s, MD, iU, aW, Y, ff, eH, eZ, r, fQ, aq, af, yq, BG, en, g, as, t, eF, bz, cm, ik, LB, qJ, nB, bE, Qu, dh, X, cj, pg, dy, m, y, B); #c1(PTK2) c2(XP_011515500) c3(focal adhesion kinase 1 isoform X16) c4( ) c5(308336, 308337, 308338, 308339, 308340, 308341) c6( ) c7(T, ht, aA, Bm, n, cz, bB, fr, ci, wo, fd, zP, V, iJ, q, ad, eA, hX, eJ, ip, av, iu, jz, yt, Dr, ds, aT, h, lG, cy, hP, K, NM, ae, dx, hm, aa, d, s, MD, iU, aW, Y, ff, eH, eZ, r, fQ, aq, aF, yq, BG, en, g, as, t, eF, bz, cm, ik, LB, qJ, nB, bE, Qu, dh, X, cj, pg, dy, m, y, B); #c1(PTK2B) c2(NP_775267) c3(protein-tyrosine kinase 2-beta isoform b) c4(249709) c5(249710, 249711, 249712, 249713, 249714, 249715, 249716, 249717, 249718, 249719, 249720, 249721, 249722, 249723) c6( ) c7(T, fd, aE, dE, K, eL, aW, Qb, g, cm, eM, n, aj, fr, cd, ai, ft, bud, zf, gd, h, IG, QE, dL, bfR, anP, aq, kp, pm, hk, FV, hR, cB, B, bL, fM, dA, jx, cz, cR, C, bX, lu, eA, J, qH, ahL, aT, bK, j, ae, eu, bA, jf, iU, Y, en, iK, aDQ, ck, nM, iM, c, aHB, mf, aDR, dy, bt, id, dG, pz, hF, ad, hX, av, bj, PZ, d, je, asf, ff, bz, qa, yE, aS, ME, y, iu); #c1(PTK2B) c2(XP_005273505) c3(protein-tyrosine kinase 2-beta isoform X2) c4(284632) c5(284633, 284634, 284635, 284636, 284637, 284638) c6( ) c7(T, fd, aE, dE, K, eL, aW, Qb, g, cm, eM, n, aj, fr, cd, ai, ft, bud, zf, gd, h, lG, QE, dL, bfR, anP, aq, kp, pm, hk, FV, hR, cB, B, bL, fM, dA, jx, cz, cR, C, bX, lu, eA, J, qH, ahL, aT, bK, j, ae, eu, bA, jf, iU, Y, en, iK, aDQ, ck, nM, iM, c, aHB, mf, aDR, dy, bt, id, dG, pz, hF, ad, hX, av, bj, PZ, d, je, asf, ff, bz, qa, yE, aS, ME, y, iu); #c1(PTK2B) c2(XP_011542743) c3(protein-tyrosine kinase 2-beta isoform X1) c4(341321) c5(341322, 341323, 341324, 341325, 341326, 341327, 341328, 341329, 341330, 341331, 341332, 341333, 341334, 341335) c6( ) c7(T, fd, aE, dE, K, eL, aW, Qb, g, cm, eM, n, aj, fr, cd, ai, ft, bud, zf, gd, h, lG, QE, dL, bfR, anP, aq, kp, pm, hk, FV, hR, cB, B, bL, fM, dA, jx, cz, cR, C, bX, lu, eA, J, qH, ahl, aT, bk, j, ae, eu, bA, jf, iU, Y, en, ik, aDQ, ck, nM, iM, c, aHB, mf, aDR, dy, bt, id, dG, pz, hF, ad, hX, av, bj, PZ, d, je, asf, ff, bz, qa, yE, aS, ME, y, iu); #c1(PTK6) c2(NP_001243287) c3(protein-tyrosine kinase 6 isoform 2) c4( ) c5(90382, 90383, 90384, 90385) c6( ) c7(T, auY, aE, bE, aW, qt, ff, jh, aT, bB, it, Fb, ci, m, en, g, t, ae, X, ad); #c1(PTK6) c2(NP_005966) c3(protein-tyrosine kinase 6 isoform 1) c4(160826, 160827) c5(160828, 160829, 160830, 160831, 160832, 160833, 160834, 160835, 160836, 160837, 160838, 160839) c6( ) c7(T, auY, aE, bE, aW, qt, ff, jh, aT, bB, it, Fb, ci, m, en, g, t, ae, X, ad); #c1(PTK7) c2(NP_001257327) c3(inactive tyrosine-protein kinase 7 isoform e) c4( ) c5(95164, 95165, 95166, 95167, 95168, 95169) c6( ) c7(ck, Mb, T, eG, tb, eM, bE, aW, Y, aT, bB, ww, g, bt, bz, iu, Gc, cp, hq); #c1(PTK7) c2(NP_002812) c3(inactive tyrosine-protein kinase 7 isoform a precursor) c4(132404) c5( ) c6( ) c7(ck, Mb, T, eG, tb, eM, bE, aW, Y, aT, bB, ww, g, bt, bz, iu, Gc, cp, hq); #c1(PTK7) c2(NP_690619) c3(inactive tyrosine-protein kinase 7 isoform b precursor) c4(244937, 244938) c5( ) c6( ) c7(ck, Mb, T, eG, tb, eM, bE, aW, Y, aT, bB, ww, g, bt, bz, iu, Gc, cp, hq); #c1(PTK7) c2(NP_690620) c3(inactive tyrosine-protein kinase 7 isoform c precursor) c4(244939) c5(244940, 244941, 244942, 244943, 244944) c6( ) c7(ck, Mb, T, eG, tb, eM, bE, aW, Y, aT, bB, ww, g, bt, bz, iu, Gc, cp, hq); #c1(PTK7) c2(NP_690621) c3(inactive tyrosine-protein kinase 7 isoform d precursor) c4(244945) c5(244946, 244947, 244948, 244949, 244950, 244951, 244952, 244953, 244954, 244955, 244956, 244957, 244958) c6( ) c7(ck, Mb, T, eG, tb, eM, bE, aW, Y, aT, bB, ww, g, bt, bz, iu, Gc, cp, hq); #c1(PTK7) c2(XP_011513067) c3(inactive tyrosine-protein kinase 7 isoform X1) c4( ) c5(304746, 304747, 304748, 304749, 304750, 304751) c6( ) c7(ck, Mb, T, eG, tb, eM, bE, aW, Y, aT, bB, ww, g, bt, bz, iu, Gc, cp, hq); #c1(PTK7) c2(XP_011513068) c3(inactive tyrosine-protein kinase 7 isoform X2) c4(304752, 304753) c5(304754, 304755, 304756, 304757, 304758, 304759, 304760, 304761, 304762, 304763, 304764, 304765) c6( ) c7(ck, Mb, T, eG, tb, eM, bE, aW, Y, aT, bB, ww, g, bt, bz, iu, Gc, cp, hq); #c1(PTMA) c2(NP_001092755) c3(prothymosin alpha isoform 1) c4(39613) c5( ) c6( ) c7(T, fd, ci, q, ad, bv, av, xE, aT, h, dL, Nj, eL, ae, s, Jl, aW, fc, r, g, cm, wB, y); #c1(PTMA) c2(NP_002814) c3(prothymosin alpha isoform 2) c4(132419) c5(132420, 132421, 132422, 132423, 132424, 132425, 132426, 132427, 132428, 132429) c6( ) c7(T, fd, ci, q, ad, bv, av, xE, aT, h, dL, Nj, eL, ae, s, JI, aW, fc, r, g, cm, wB, y); #c1 (PTMA) c2(XP_011509821) c3 (prothymosin alpha isoform X2) c4(300405) c5( ) c6( ) c7(T, fd, ci, q, ad, bv, av, xE, aT, h, dL, Nj, eL, ae, s, Jl, aW, fc, r, g, cm, wB, y); #c1(PTMA) c2(XP_011509823) c3 (prothymosin alpha isoform X4) c4( ) c5(300406, 300407, 300408, 300409, 300410) c6( ) c7(T, fd, ci, q, ad, bv, av, xE, aT, h, dL, Nj, eL, ae, s, JI, aW, fc, r, g, cm, wB, y); #c1(PTMA) c2(XP_011509824) c3(prothymosin alpha isoform X5) c4( ) c5(300411, 300412, 300413, 300414) c6( ) c7(T, fd, ci, q, ad, bv, av, xE, aT, h, dL, Nj, eL, ae, s, JI, aW, fc, r, g, cm, wB, y); #c1(PTMS) c2(NP_002815) c3 (parathymosin) c4(132430, 132431) c5(132432, 132433, 132434, 132435, 132436, 132437, 132438, 132439, 132440, 132441, 132442, 132443, 132444) c6( ) c7(af); #c1(PTMS) c2(XP_011519289) c3(parathymosin isoform X1) c4(313927) c5(313928, 313929, 313930, 313931, 313932) c6( ) c7(af); #c1(PTN) c2(XP_005250572) c3(pleiotrophin isoform X1) c4(270404) c5( ) c6( ) c7(T, dD, aC, ea, if, al, jh, fd, ajp, j, ci, eq, Lw, qN, zP, cd, Pu, ad, er, dP, eA, av, Pv, ft, PC, aeO, aT, iv, h, bl, zg, nJ, ae, bA, Pw, zQ, d, aeR, aW, Y, eZ, r, g, as, cm, bz, fq, sS, beH, beJ, eM, dB, y); #c1(PTN) c2(XP_005250573) c3(pleiotrophin isoform X2) c4( ) c5(270405, 270406, 270407, 270408, 270409, 270410) c6( ) c7(T, dD, aC, ea, if, al, jh, fd, ajp, j, ci, eq, Lw, qN, zP, cd, Pu, ad, er, dP, eA, av, Pv, ft, PC, aeO, aT, iv, h, bl, zg, nJ, ae, bA, Pw, zQ, d, aeR, aW, Y, eZ, r, g, as, cm, bz, fq, sS, beH, beJ, eM, dB, y); #c1(PTOV1) c2(NP_059128) c3(prostate tumor-overexpressed gene 1 protein) c4(193195) c5(193196, 193197, 193198, 193199, 193200, 193201) c6( ) c7(aet, aA, cz, ci, g, Eh, ae, ad); #c1(PTP4A1) c2(XP_011534414) c3(protein tyrosine phosphatase type IVA 1 isoform XI) c4(330903, 330904) c5(330905, 330906, 330907, 330908, 330909, 330910, 330911, 330912, 330913, 330914, 330915, 330916, 330917, 330918, 330919, 330920) c6( ) c7(T, fM, aW, ff, aT, it, By, ey, en, g, t, bz); #c1(PTP4A3) c2(XP_005250824) c3(protein tyrosine phosphatase type IVA 3 isoform X3) c4(270586) c5(270587, 270588, 270589, 270590, 270591, 270592, 270593, 270594, 270595, 270596, 270597, 270598, 270599, 270600, 270601, 270602) c6( ) c7(T, aA, bB, n, fd, oH, ci, q, eA, hX, av, aT, cp, cY, B, iU, aW, Y, ff, r, aF, en, g, t, cm, bz, iP, ck, bE, bt, it, X); #c1(PTP4A3) c2(XP_011544863) c3(protein tyrosine phosphatase type IVA 3 isoform X1) c4(343854, 343855) c5(343856, 343857, 343858, 343859, 343860, 343861, 343862, 343863, 343864, 343865) c6( ) c7(T, aA, bB, n, fd, oH, ci, q, eA, hX, av, al, cp, cY, B, iU, aW, Y, ff, r, af, en, g, t, cm, bz, iP, ck, bE, bt, it, X); #c1(PTPMT1) c2(NP_001137456) c3(phosphatidylglycerophosphatase and protein-tyrosine phosphatase 1 isoform 2) c4(55799) c5(55800, 55801, 55802, 55803, 55804, 55805, 55806) c6( ) c7(cm, T); #c1(PTPMT1) c2(NP_783859) c3(phosphatidylglycerophosphatase and protein-tyrosine phosphatase 1 isoform 1) c4(251757) c5(251758, 251759, 251760, 251761, 251762, 251763) c6( ) c7(cm, T); #c1(PTPN1) c2(NP_001265547) c3(tyrosine-protein phosphatase non-receptor type 1 isoform 2) c4( ) c5(98823, 98824, 98825, 98826, 98827) c6( ) c7(bL, T, dD, bKq, fM, eR, yS, ea, iW, eX, jh, aA, cz, n, aj, v, j, bB, eq, lR, iJ, q, J, ad, eA, ady, ku, vJ, nX, bWr, aT, jy, vc, eV, K, qM, ae, af, sj, aa, A, B, iU, aW, oV, blP, r, dq, aq, aF, t, KM, pb, qa, bJ, fT, sS, bE, vy, at, GN, mf, m, ei, bt, it, X); #c1(PTPN1) c2(NP_002818) c3(tyrosine-protein phosphatase non-receptor type 1 isoform 1) c4(132456, 132457) c5(132458, 132459, 132460, 132461, 132462, 132463, 132464) c6( ) c7(bL, T, dD, bKq, fM, eR, yS, ea, iW, ex, jh, aA, cz, n, aj, v, j, bB, eq, lR, iJ, q, J, ad, eA, ady, ku, vJ, nX, bWr, aT, jy, vc, eV, K, qM, ae, af, sj, aa, A, B, iU, aW, oV, blP, r, dq, aq, aF, t, KM, pb, qa, bJ, fT, sS, bE, vy, at, GN, mf, m, ei, bt, it, X); #c1(PTPN1I) c2(NP_002825) c3(tyrosine-protein phosphatase non-receptor type fl isoform 1) c4(132512, 132513) c5(132514, 132515, 132516, 132517, 132518, 132519) c6( ) c7(ID, bM, T, aC, fR, apr, yS, azi, if, e, bB, ci, cxu, x, bv, aE, Vv, K, ly, aW, az, Az, hl, aGQ, kt, Au, aB, g, as, cm, bw, fT, eM, dc, aiM, nw, k, Ro, NK, kv, qN, ckn, ai, md, b, cc, Bj, cU, h, eV, hn, Dg, A, bIP, fe, mo, lx, iO, kT, aNd, bD, B, Dw, fM, jx, eX, aA, afK, v, yO, cdP, aDO, aNe, AO, eA, akE, Mg, aup, nX, aT, cxs, aut, dz, hK, rz, s, iU, iZ, Y, bEc, lo, en, bQ, boW, ck, kP, auo, mG, AH, Uq, bt, xb, cxr, acl, atS, VT, jh, oH, cxt, hF, cxq, ds, vc, ji, pq, cjs, atJ, d, TY, et, qt, ff, aEW, aF, Sm, iV, bz, Ew, bE, gZ, dX, rH, i, y); #c1(PTPN11) c2(NP_542168) c3(tyrosine-protein phosphatase non-receptor type Il isoform 2) c4( ) c5(232164, 232165, 232166, 232167, 232168, 232169, 232170, 232171, 232172, 232173, 232174, 232175) c6( ) c7(ID, bM, T, aC, fR, apr, yS, azi, if, e, bB, ci, cxu, x, bv, aE, Vv, K, ly, aW, az, Az, hl, aGQ, kt, Au, aB, g, as, cm, bw, fT, eM, dc, aiM, nw, k, Ro, NK, kv, qN, ckn, ai, md, b, cc, Bj, cU, h, eV, hn, Dg, A, bIP, fe, mo, lx, iO, kT, aNd, bD, B, Dw, fM, jx, eX, aA, afK, v, yO, cdP, aDO, aNe, AD, eA, akE, Mg, aup, nX, aT, cxs, aut, dz, hK, rz, s, iU, iZ, Y, bEc, lo, en, bQ, boW, ck, kP, auo, mG, AH, Uq, bt, xb, cxr, acl, atS, VT, jh, oH, cxt, hF, cxq, ds, vc, ji, pq, cjs, atJ, d, TY, et, qt, ff, aEW, aF, Sm, iV, bz, Ew, bE, gZ, dX, rH, i, y); #c1(PTPN11) c2(XP_006719589) c3(tyrosine-protein phosphatase non-receptor type 11 isoform X1) c4(292661) c5( ) c6( ) c7(ID, bM, T, aC, fR, apr, yS, azi, if, e, bB, ci, cxu, x, bv, aE, Vv, K, ly, aW, az, Az, hl, aGQ, kt, Au, aB, g, as, cm, bw, fT, eM, dc, aiM, nw, k, Ro, NK, kv, qN, ckn, ai, md, b, cc, Bj, cU, h, eV, hn, Dg, A, bIP, fe, mo, lx, iO, kT, aNd, bD, B, Dw, fM, jx, eX, aA, afK, v, yO, cdP, aDO, aNe, AO, eA, akE, Mg, aup, nX, aT, cxs, aut, dz, hK, rz, s, iU, iZ, Y, bEc, lo, en, bl, boW, ck, kP, auo, mG, AH, Uq, bt, xb, cxr, acl, atS, VT, jh, oH, cxt, hF, cxq, ds, vc, ji, pq, cjs, atJ, d, TY, et, qt, ff, aEW, aF, Sm, iV, bz, Ew, bE, gZ, dX, rH, i, y); #c1(PTPN12) c2(NP_001124480) c3(tyrosine-protein phosphatase non-receptor type 12 isoform 2) c4( ) c5(49139, 49140, 49141, 49142, 49143, 49144, 49145, 49146, 49147, 49148) c6( ) c7(ck, T, kP, mP, bE, aW, aT, bB, n, hD, K, ci, oQ, it, B, ad); #c1(PTPN12) c2(NP_002826) c3(tyrosine-protein phosphatase non-receptor type 12 isoform 1) c4(132520, 132521, 132522) c5(132523, 132524, 132525, 132526, 132527, 132528, 132529, 132530) c6( ) c7(ck, T, kP, mP, bE, aW, al, bB, n, hD, K, ci, oQ, it, B, ad); #c1(PTPN12) c2(XP_005250575) c3(tyrosine-protein phosphatase non-receptor type 12 isoform X2) c4( ) c5(270411, 270412, 270413, 270414, 270415) c6( ) c7(ck, T, kP, mP, bE, aW, aT, bB, n, hD, K, ci, oQ, it, B, ad); #c1(PTPN12) c2(XP_006716136) c3(tyrosine-protein phosphatase non-receptor type 12 isoform X1) c4(290089) c5(290090) c6( ) c7(ck, T, kP, mP, bE, aW, aT, bB, n, hD, K, ci, ol, it, B, ad); #c1(PTPN13) c2(NP_006255) c3(tyrosine-protein phosphatase non-receptor type 13 isoform 2) c4(163321, 163322) c5( ) c6( ) c7(T, ob, wu, ht, aA, bB, hy, ci, bX, q, ad, alZ, av, cxw, aE, aT, rO, wm, H, ae, dx, mH, je, aW, ev, ff, eZ, r, en, as, iK, bz, ck, iO, cxv, rR, iM, eM, Kv, bE, Qu, U, aiS, KY, B); #c1(PTPN13) c2(NP_542416) c3(tyrosine-protein phosphatase non-receptor type 13 isoform 4) c4(232795) c5( ) c6( ) c7(T, ob, wu, ht, aA, bB, hy, ci, bX, q, ad, alZ, av, cxw, aE, aT, rO, wm, H, ae, dx, mH, je, aW, ev, ff, eL, r, en, as, iK, bz, ck, iO, cxv, rR, iM, eM, Kv, bE, Qu, U, aiS, KY, B); #c1(PTPN13) c2(XP_005263224) c3(tyrosine-protein phosphatase non-receptor type 13 isoform X6) c4(278366) c5(278367, 278368, 278369, 278370, 278371, 278372) c6( ) c7(T, ob, wu, ht, aA, bB, hy, ci, bX, q, ad, alZ, av, cxw, aE, aT, rO, wm, H, ae, dx, mH, je, aW, ev, ff, eZ, r, en, as, ik, bz, ck, iO, cxv, rR, iM, eM, Kv, bE, Qu, U, aiS, KY, B); #c1(PTPN13) c2(XP_011530465) c3(tyrosine-protein phosphatase non-receptor type 13 isoform X1) c4( ) c5(326334, 326335, 326336, 326337, 326338, 326339) c6( ) c7(T, ob, wu, ht, aA, bB, hy, ci, bX, q, ad, alZ, av, cxw, aE, aT, rO, wm, H, ae, dx, mH, je, aW, ev, ff, eZ, r, en, as, ik, bz, ck, iO, cxv, rR, iM, eM, Kv, bE, Qu, U, aiS, KY, B); #c1(PTPN13) c2(XP_011530468) c3(tyrosine-protein phosphatase non-receptor type 13 isoform X4) c4( ) c5(326340, 326341, 326342, 326343, 326344, 326345, 326346, 326347, 326348, 326349) c6( ) c7(T, ob, wu, ht, aA, bB, hy, ci, bX, q, ad, alZ, av, cxw, aE, aT, rO, wm, H, ae, dx, mH, je, aW, ev, ff, eL, r, en, as, iK, bz, ck, iO, cxv, rR, iM, eM, Kv, bE, Qu, U, aiS, KY, B); #c1(PTPN13) c2(XP_011530470) c3(tyrosine-protein phosphatase non-receptor type 13 isoform X7) c4(326350) c5( ) c6( ) c7(T, ob, wu, ht, aA, bB, hy, ci, bX, q, ad, alZ, av, cxw, aE, aT, rO, wm, H, ae, dx, mH, je, aW, ev, ff, eZ, r, en, as, ik, bz, ck, iO, cxv, rR, iM, eM, Kv, bE, Qu, U, aiS, KY, B); #c1(PTPN13) c2(XP_011530471) c3(tyrosine-protein phosphatase non-receptor type 13 isoform X8) c4( ) c5(326351, 326352, 326353, 326354, 326355, 326356, 326357, 326358, 326359, 326360, 326361, 326362) c6( ) c7(T, ob, wu, ht, aA, bB, hy, ci, bX, q, ad, alZ, av, cxw, aE, aT, rl, wm, H, ae, dx, mH, je, aW, ev, ff, eZ, r, en, as, ik, bz, ck, iO, cxv, rR, iM, eM, Kv, bE, Qu, U, aiS, KY, B); #c1(PTPN13) c2(XP_011530474) c3(tyrosine-protein phosphatase non-receptor type 13 isoform X11) c4(326363) c5(326364, 326365, 326366, 326367, 326368, 326369) c6( ) c7(T, ob, wu, ht, aA, bB, hy, ci, bX, q, ad, alZ, av, cxw, aE, aT, rO, wm, H, ae, dx, mH, je, aW, ev, ff, eL, r, en, as, ik, bz, ck, iQ, cxv, rR, iM, eM, Kv, bE, Qu, U, aiS, KY, B); #c1(PTPN14) c2(NP_005392) c3(tyrosine-protein phosphatase non-receptor type 14) c4(155679, 155680) c5(155681, 155682, 155683, 155684, 155685, 155686, 155687, 155688, 155689, 155690, 155691, 155692) c6( ) c7(T, r, Vv, aA, cxx, cxy, q, B); #c1(PTPN18) c2(NP_001135842) c3(tyrosine-protein phosphatase non-receptor type 18 isoform 2) c4( ) c5(53414, 53415, 53416, 53417, 53418, 53419) c6( ) c7(aW, fb, aT); #c1(PTPN18) c2(NP_055184) c3(tyrosine-protein phosphatase non-receptor type 18 isoform 1) c4(179646) c5(179647, 179648, 179649, 179650, 179651, 179652) c6( ) c7(aW, fb, aT); #c1(PTPN18) c2(XP_006712479) c3 (tyrosine-protein phosphatase non-receptor type 18 isoform X1) c4(286683) c5( ) c6( ) c7(aW, fb, aT); #c1(PTPN2) c2(NP_001193942) c3(tyrosine-protein phosphatase non-receptor type 2 isoform 4) c4(83284) c5(83285, 83286, 83287, 83288, 83289, 83290) c6( ) c7(bL, xN, T, lo, jh, cl, aj, ai, o, J, ady, ju, kH, aT, ji, hK, A, d, iU, aW, eZ, aMA, fe, hp, iV, dr, ck, kP, yH, ei, y, ra); #c1(PTPN2) c2(NP_002819) c3(tyrosine-protein phosphatase non-receptor type 2 isoform 1) c4(132465) c5(132466, 132467, 132468, 132469, 132470, 132471) c6( ) c7(bL, xN, T, lo, jh, cl, aj, ai, o, J, ady, ju, kH, aT, ji, hK, A, d, iU, aW, eZ, aMA, fe, hp, iV, dr, ck, kP, yH, ei, y, ra); #c1(PTPN2) c2(NP_536347) c3(tyrosine-protein phosphatase non-receptor type 2 isoform 2) c4(231249) c5(231250, 231251, 231252, 231253, 231254, 231255) c6( ) c7(bL, xN, T, lo, jh, cl, aj, ai, o, J, ady, ju, kH, aT, ji, hK, A, d, iU, aW, eZ, aMA, fe, hp, iV, dr, ck, kP, yH, ei, y, ra); #c1(PTPN2) c2(NP_536348) c3(tyrosine-protein phosphatase non-receptor type 2 isoform 3) c4(231256) c5(231257, 231258, 231259, 231260, 231261, 231262) c6( ) c7(bL, xN, T, lo, jh, cl, aj, ai, o, J, ady, ju, kH, aT, ji, hK, A, d, iU, aW, eZ, aMA, fe, hp, iV, dr, ck, kP, yH, ei, y, ra); #c1(PTPN21) c2(XP_011534669) c3(tyrosine-protein phosphatase non-receptor type 21 isoform X1) c4(331167, 331168) c5(331169, 331170, 331171, 331172, 331173, 331174, 331175, 331176, 331177, 331178, 331179, 331180, 331181, 331182, 331183, 331184) c6( ) c7(hD, T); #c1(PTPN21) c2(XP_011534671) c3(tyrosine-protein phosphatase non-receptor type 21 isoform X3) c4( ) c5(331185) c6( ) c7(hD, T); #c1(PTPN22) c2(XP_011539526) c3(tyrosine-protein phosphatase non-receptor type 22 isoform X5) c4(337724) c5(337725, 337726, 337727, 337728, 337729, 337730) c6( ) c7(oO, T, bzx, fR, ea, e, cl, aoj, qj, yM, bv, AW, xy, vV, dE, K, DY, hH, bc, bn, aW, asD, HA, hN, pl, ve, yl, lt, Jr, Fh, cj, uo, dW, dD, sQ, lo, yG, fN, aj, ai, h, DU, ey, nP, A, mR, OX, lb, ge, fe, W, Pa, ut, fL, Pr, B, bL, eR, jx, cz, cR, hz, j, V, pd, o, J, ga, mP, aT, hk, DX, af, hM, Y, lC, apK, pb, lY, ans, OW, ac, ig, yW, hr, wR, dy, gT, dM, eq, mC, Uy, ad, wD, dP, vJ, qh, hD, ji, lQ, yF, cr, eZ, dN, Mz, hp, iV, aYt, Bv, zr, axs, Lj, atM, z, agl, ol, y, hv, pC); #c1(PTPN23) c2(NP_001291411) c3(tyrosine-protein phosphatase non-receptor type 23 isoform 2) c4( ) c5(115958, 115959, 115960, 115961, 115962, 115963) c6( ) c7(h, aW, vT, aT); #c1(PTPN23) c2(NP_056281) c3(tyrosine-protein phosphatase non-receptor type 23 isoform 1) c4(186929) c5(186930, 186931, 186932, 186933, 186934, 186935) c6( ) c7(h, aW, vT, aT); #c1(PTPN23) c2(XP_005265088) c3(tyrosine-protein phosphatase non-receptor type 23 isoform X1) c4(279427) c5(279428, 279429, 279430, 279431, 279432, 279433) c6( ) c7(h, aW, vT, aT); #c1(PTPN3) c2(NP_001138840) c3(tyrosine-protein phosphatase non-receptor type 3 isoform 2) c4(57923) c5( ) c6( ) c7(Mb, T, jz, asW, eM, bE, aW, aT, bB, n, aq, K, YH, cm, B, hX); #c1(PTPN3) c2(NP_001138842) c3(tyrosine-protein phosphatase non-receptor type 3 isoform 4) c4(57924, 57925) c5( ) c6( ) c7(Mb, T, jz, asW, eM, bE, aW, aT, bB, n, aq, K, YH, cm, B, hX); #c1(PTPN3) c2(NP_001138844) c3(tyrosine-protein phosphatase non-receptor type 3 isoform 6) c4( ) c5(57926, 57927, 57928, 57929, 57930) c6( ) c7(Mb, T, jz, asW, eM, bE, aW, aT, bB, n, aq, K, YH, cm, B, hX); #c1(PTPN3) c2(XP_006717260) c3(tyrosine-protein phosphatase non-receptor type 3 isoform X4) c4(290841) c5(290842, 290843) c6( ) c7(Mb, T, jz, asW, eM, bE, aW, aT, bB, n, aq, K, YH, cm, B, hX); #c1(PTPN3) c2(XP_006717264) c3(tyrosine-protein phosphatase non-receptor type 3 isoform X6) c4(290844) c5(290845, 290846, 290847, 290848, 290849, 290850) c6( ) c7(Mb, T, jz, asW, eM, bE, aW, aT, bB, n, aq, K, YH, cm, B, hX); #c1(PTPN3) c2(XP_006717266) c3(tyrosine-protein phosphatase non-receptor type 3 isoform X7) c4( ) c5(290851) c6( ) c7(Mb, T, jz, asW, eM, bE, aW, aT, bB, n, aq, K, YH, cm, B, hX); #c1(PTPN3) c2(XP_006717267) c3(tyrosine-protein phosphatase non-receptor type 3 isoform X2) c4( ) c5(290852, 290853, 290854, 290855, 290856, 290857) c6( ) c7(Mb, T, jz, asW, eM, bE, aW, aT, bB, n, aq, K, YH, cm, B, hX); #c1(PTPN3) c2(XP_011517191) c3(tyrosine-protein phosphatase non-receptor type 3 isoform X1) c4( ) c5(310787, 310788, 310789, 310790, 310791, 310792, 310793, 310794) c6( ) c7(Mb, T, jz, asW, eM, bE, aW, aT, bB, n, aq, K, YH, cm, B, hX); #c1(PTPN3) c2(XP_011517192) c3(tyrosine-protein phosphatase non-receptor type 3 isoform X5) c4( ) c5(310795, 310796, 310797, 310798, 310799, 310800) c6( ) c7(Mb, T, jz, asW, eM, bE, aW, aT, bB, n, aq, K, YH, cm, B, hX); #c1(PTPN4) c2(NP_002821) c3 (tyrosine-protein phosphatase non-receptor type 4) c4(132472, 132473) c5(132474, 132475, 132476, 132477, 132478, 132479) c6( ) c7(ck, iQ, pg, iY, rz, B); #c1(PTPN4) c2(XP_011509862) c3(tyrosine-protein phosphatase non-receptor type 4 isoform X1) c4(300448) c5(300449, 300450, 300451, 300452, 300453, 300454) c6( ) c7(ck, iU, pg, iY, rz, B); #c1(PTPN4) c2(XP_011509864) c3(tyrosine-protein phosphatase non-receptor type 4 isoform X3) c4(300455) c5(300456, 300457, 300458, 300459, 300460, 300461) c6( ) c7(ck, iQ, pg, iY, rz, B); #c1(PTPN4) c2(XP_011509865) c3(tyrosine-protein phosphatase non-receptor type 4 isoform X4) c4( ) c5(300462, 300463, 300464, 300465, 300466, 300467) c6( ) c7(ck, iU, pg, iY, rz, B); #c1(PTPN5) c2(NP_001265165) c3(tyrosine-protein phosphatase non-receptor type 5 isoform b) c4(97905) c5(97906, 97907, 97908, 97909, 97910, 97911) c6( ) c7(HC, bO, cm, h, yq, bK, K, m, t); #c1(PTPN5) c2(NP_008837) c3(tyrosine-protein phosphatase non-receptor type 5 isoform a) c4(168952) c5(168953, 168954, 168955, 168956, 168957, 168958, 168959, 168960, 168961, 168962) c6( ) c7(HC, b, cm, h, yq, bK, K, m, t); #c1(PTPN6) c2(NP_002822) c3(tyrosine-protein phosphatase non-receptor type 6 isoform 1) c4(132480, 132481) c5(132482, 132483, 132484, 132485, 132486, 132487) c6( ) c7(bM, T, fM, ow, jh, aA, bB, im, afK, aj, v, ci, fd, pz, hF, V, cxz, q, J, ad, eA, x, ady, bvG, oW, aT, Ne, kH, cy, dz, jy, K, lQ, h, zy, pk, pq, ae, af, je, iU, up, et, aW, pr, qY, eH, ff, cr, r, eZ, aF, en, as, eE, t, eF, nN, GR, dr, yR, pm, ck, bw, eU, lt, iM, eM, bE, Jp, aOL, cj, eo, m, k, lS, bt, B); #c1(PTPN6) c2(NP_536859) c3(tyrosine-protein phosphatase non-receptor type 6 isoform 3) c4(231382) c5(231383, 231384, 231385, 231386, 231387, 231388) c6( ) c7(bM, T, fM, ow, jh, aA, bB, im, afK, aj, v, ci, fd, pz, hF, V, cxz, q, J, ad, eA, x, ady, bvG, oW, aT, Ne, KH, cy, dz, jy, K, lQ, h, zy, pk, pq, ae, af, je, iU, up, et, aW, pr, qY, eH, ff, cr, r, eZ, af, en, as, eE, t, eF, nN, GR, dr, yR, pm, ck, bw, eU, lt, iM, eM, bE, Jp, aOL, cj, eo, m, k, lS, bt, B); #c1(PTPN6) c2(XP_011519290) c3(tyrosine-protein phosphatase non-receptor type 6 isoform X1) c4(313933) c5(313934, 313935, 313936, 313937, 313938, 313939, 313940, 313941, 313942, 313943, 313944, 313945) c6( ) c7(bM, T, fM, ow, jh, aA, bB, im, afK, aj, v, ci, fd, pz, hF, V, cxz, q, J, ad, eA, x, ady, bvG, oW, aT, Ne, kH, cy, dz, jy, K, lQ, h, zy, pk, pq, ae, af, je, iU, up, et, aW, pr, qY, eH, ff, cr, r, eZ, aF, en, as, BE, t, eF, nN, GR, dr, yR, pm, ck, bw, eU, lt, iM, eM, bE, Jp, aOL, cj, eo, m, k, lS, bt, B); #c1(PTPN7) c2(NP_001186726) c3(tyrosine-protein phosphatase non-receptor type 7 isoform 3) c4(78413) c5(78414, 78415, 78416, 78417, 78418, 78419, 78420) c6( ) c7(T, ol, K, hf, ae, ad); #c1(PTPN7) c2(NP_002823) c3(tyrosine-protein phosphatase non-receptor type 7 isoform 1) c4(132488, 132489) c5(132490, 132491, 132492, 132493, 132494, 132495, 132496, 132497) c6( ) c7(T, ol, K, hf, ae, ad); #c1(PTPN7) c2(NP_542155) c3(tyrosine-protein phosphatase non-receptor type 7 isoform 2) c4(231416, 231417, 231418) c5(231419, 231420, 231421, 231422, 231423, 231424, 231425, 231426, 231427, 231428, 231429, 231430, 231431, 231432, 231433, 231434, 231435, 231436, 231437, 231438, 231439, 231440, 231441, 231442, 231443, 231444) c6( ) c7(T, ol, K, hf, ae, ad); #c1(PTPN7) c2(XP_011508125) c3(tyrosine-protein phosphatase non-receptor type 7 isoform X4) c4(298602, 298603) c5(298604, 298605, 298606, 298607, 298608, 298609, 298610, 298611, 298612, 298613, 298614, 298615) c6( ) c7(T, ol, K, hf, ae, ad); #c1(PTPN9) c2(NP_002824) c3(tyrosine-protein phosphatase non-receptor type 9) c4(132498, 132499) c5(132500, 132501, 132502, 132503, 132504, 132505, 132506, 132507, 132508, 132509, 132510, 132511) c6( ) c7(bL, aW, dG, pm, aT); #c1(PTPRA) c2(NP_002827) c3(receptor-type tyrosine-protein phosphatase alpha isoform 1 precursor) c4(132531, 132532, 132533, 132534, 132535, 132536, 132537) c5(132538, 132539, 132540, 132541, 132542, 132543, 132544, 132545, 132546, 132547, 132548, 132549, 132550, 132551, 132552, 132553, 132554, 132555, 132556, 132557, 132558, 132559, 132560, 132561, 132562, 132563, 132564, 132565) c6( ) c7(bL, T, aw, aA, fd, j, ci, hF, V, x, xc, kH, aT, cy, K, de, af, je, aW, hf, cl, cm, bz, QA, ck, iM, eM, bE, uz, m, k, bt); #c1(PTPRA) c2(NP_543030) c3(receptor-type tyrosine-protein phosphatase alpha isoform 2 precursor) c4(233301, 233302) c5(233303, 233304, 233305, 233306, 233307, 233308, 233309, 233310, 233311, 233312, 233313, 233314, 233315, 233316, 233317, 233318, 233319, 233320, 233321, 233322, 233323, 233324, 233325, 233326, 233327, 233328, 233329, 233330, 233331, 233332, 233333, 233334, 233335, 233336, 233337, 233338, 233339, 233340) c6( ) c7(bL, T, aw, aA, fd, j, ci, hF, V, x, xc, kH, aT, cy, K, dQ, af, je, aW, hf, cl, cm, bz, QA, ck, iM, eM, bE, uz, m, k, bt); #c1(PTPRB) c2(NP_001103224) c3 (receptor-type tyrosine-protein phosphatase beta isoform a) c4(41851) c5(41852, 41853, 41854, 41855, 41856, 41857) c6( ) c7(Vy, lB, FT, aT); #c1(PTPRB) c2(NP_001193900) c3(receptor-type tyrosine-protein phosphatase beta isoform c precursor) c4(83100, 83101) c5(83102, 83103, 83104, 83105, 83106, 83107, 83108, 83109, 83110, 83111, 83112, 83113, 83114) c6( ) c7(Vy, lB, FT, aT); #c1(PTPRB) c2(NP_001193901) c3(receptor-type tyrosine-protein phosphatase beta isoform d precursor) c4(83115) c5(83116, 83117, 83118, 83119, 83120, 83121) c6( ) c7(Vy, lB, FT, aT); #c1(PTPRB) c2(NP_002828) c3(receptor-type tyrosine-protein phosphatase beta isoform b precursor) c4(132566) c5(132567, 132568, 132569, 132570, 132571, 132572) c6( ) c7(Vy, lB, FT, aT); #c1(PTPRB) c2(XP_011536916) c3(receptor-type tyrosine-protein phosphatase beta isoform X2) c4(334586) c5(334587, 334588, 334589, 334590, 334591) c6( ) c7(Vy, lB, FT, aT); #c1(PTPRC) c2(NP_001254727) c3(receptor-type tyrosine-protein phosphatase C isoform 5 precursor) c4( ) c5(94890, 94891, 94892, 94893, 94894, 94895) c6( ) c7(oO, T, fR, pf, aAx, l, fd, ci, bh, ip, aao, x, bv, aE, K, H, pk, aiV, zC, Fu, cm, bw, GR, ou, cj, zv, k, aw, aj, br, Nh, h, hn, ey, bxg, A, CM, fe, Og, kp, yq, Wt, asE, yR, Qf, ajd, fL, B, L, fM, dA, fG, jx, aA, j, V, cxA, q, J, pd, ae, amO, af, hM, bf, nO, oV, Y, r, awu, nC, pb, ck, iM, dy, yH, bt, vi, iW, jh, bs, aGx, wV, uu, pz, ad, hX, Jm, bTr, Dq, LA, hD, ji, acQ, MG, bdd, je, lR, Of, eZ, gl, aF, Z, aDw, TA, LB, aS, y); #c1(PTPRC) c2(NP_002829) c3(receptor-type tyrosine-protein phosphatase C isoform 1 precursor) c4(132573, 132574, 132575) c5(132576, 132577, 132578, 132579, 132580, 132581, 132582, 132583, 132584, 132585, 132586, 132587, 132588, 132589, 132590, 132591, 132592, 132593) c6( ) c7(oO, T, fR, pf, aAx, l, fd, ci, bh, ip, aao, x, bv, aE, K, H, pk, aiV, zC, Fu, cm, bw, GR, ou, cj, zv, k, aw, aj, br, Nh, h, hn, ey, bxg, A, CM, fe, Og, kp, yq, Wt, asE, yR, Qf, ajd, fL, B, L, fM, dA, fG, jx, aA, j, V, cxA, q, J, pd, ae, amO, af, hM, bf, nO, oV, Y, r, awu, nC, pb, ck, iM, dy, yH, bt, vi, iW, jh, bs, aGx, wV, uu, pz, ad, hX, Jm, bTr, Dq, LA, hD, ji, acQ, MG, bdd, je, lR, Of, eZ, gl, aF, Z, aDw, TA, LB, aS, y); #c1(PTPRC) c2(NP_563578) c3(receptor-type tyrosine-protein phosphatase C isoform 2 precursor) c4(233522, 233523) c5(233524, 233525, 233526, 233527, 233528, 233529, 233530, 233531, 233532, 233533, 233534, 233535, 233536, 233537, 233538, 233539) c6( ) c7(oO, T, FR, pf, aAx, l, fd, ci, bh, ip, aao, x, bv, aE, K, H, pk, aiV, zC, Fu, cm, bw, GR, ou, cj, zv, k, aw, aj, br, Nh, h, hn, ey, bxg, A, CM, fe, Og, kp, yq, Wt, asE, yR, Qf, ajd, fL, B, L, fM, dA, fG, jx, aA, j, V, cxA, q, J, pd, ae, amO, af, hM, bf, nO, oV, Y, r, awu, nC, pb, ck, iM, dy, yH, bt, vi, iW, jh, bs, aGx, wV, uu, pz, ad, hX, Jm, bTr, Dq, LA, HD, ji, acQ, MG, bdd, je, lR, Of, eZ, gl, aF, Z, aDw, TA, LB, aS, y); #c1(PTPRC) c2(XP_006711535) c3(receptor-type tyrosine-protein phosphatase C isoform X1) c4(286069, 286070, 286071) c5(286072, 286073, 286074, 286075, 286076, 286077, 286078, 286079, 286080, 286081, 286082, 286083, 286084, 286085, 286086, 286087, 286088, 286089) c6( ) c7(oO, T, fR, pf, aAx, l, fd, ci, bh, ip, aao, x, bv, aE, K, H, pk, aiV, zC, Fu, cm, bw, GR, ou, cj, zv, k, aw, aj, br, Nh, h, hn, ey, bxg, A, CM, fe, Og, kp, yq, Wt, asE, yR, Of, ajd, FL, B, L, fM, dA, fG, jx, aA, j, V, cxA, q, J, pd, ae, amO, af, hM, bf, nO, oV, Y, r, awu, nC, pb, ck, iM, dy, yH, bt, vi, iW, jh, bs, aGx, wV, uu, pz, ad, hX, Jm, bTr, Dq, LA, hD, ji, ach, MG, bdd, je, lR, Of, eZ, gl, aF, Z, aDw, TA, LB, aS, y); #c1(PTPRC) c2(XP_006711536) c3(receptor-type tyrosine-protein phosphatase C isoform X2) c4(286090, 286091) c5(286092, 286093, 286094, 286095, 286096, 286097, 286098, 286099, 286100, 286101, 286102, 286103) c6( ) c7(oO, T, FR, pf, aAx, l, fd, ci, bh, ip, aao, x, bv, aE, K, H, pk, aiV, zC, Fu, cm, bw, GR, ou, cj, zv, k, aw, aj, br, Nh, h, hn, ey, bxg, A, CM, fe, Og, kp, yq, Wt, asE, yR, Qf, ajd, fL, B, L, fM, dA, fG, jx, aA, j, V, cxA, q, J, pd, ae, amO, af, hM, bf, nO, oV, Y, r, awu, nC, pb, ck, iM, dy, yH, bt, vi, iW, jh, bs, aGx, wV, uu, pz, ad, hX, Jm, bTr, Dq, LA, hD, ji, ach, MG, bdd, je, lR, Of, eZ, gl, aF, Z, aDw, TA, LB, aS, y); #c1(PTPRC) c2(XP_006711537) c3(receptor-type tyrosine-protein phosphatase C isoform X3) c4(286104, 286105) c5(286106, 286107, 286108, 286109, 286110, 286111) c6( ) c7(oO, T, FR, pf, aAx, l, fd, ci, bh, ip, aao, x, bv, aE, K, H, pk, aiV, zC, Fu, cm, bw, GR, ou, cj, zv, k, aw, aj, br, Nh, h, hn, ey, bxg, A, CM, fe, Og, kp, yq, Wt, asE, yR, Qf, ajd, fL, B, L, fM, dA, fG, jx, aA, j, V, cxA, q, J, pd, ae, amO, af, hM, bf, nO, oV, Y, r, awu, nC, pb, ck, iM, dy, yH, bt, vi, iW, jh, bs, aGx, wV, uu, pz, ad, hX, Jm, bTr, Dq, LA, hD, ji, acQ, MG, bdd, je, lR, Of, eZ, gl, aF, Z, aDw, TA, LB, aS, y); #c1(PTPRCAP) c2(NP_005599) c3(protein tyrosine phosphatase receptor type C-associated protein precursor) c4(157867) c5(157868, 157869, 157870, 157871, 157872, 157873) c6( ) c7(V, aF, aA); #c1(PTPRD) c2(NP_001035802) c3(receptor-type tyrosine-protein phosphatase delta isoform 5 precursor) c4(33123, 33124) c5(33125, 33126, 33127, 33128, 33129, 33130, 33131, 33132, 33133, 33134, 33135, 33136) c6( ) c7(bL, bM, T, dD, yY, EQ, cz, lr, aj, v, j, eq, cd, J, cF, pN, hb, aT, jy, d, ff, mQ, Ej, en, cm, bz, fq, abV, ac, cxB, adg); #c1(PTPRD) c2(NP_001164496) c3(receptor-type tyrosine-protein phosphatase delta isoform 6 precursor) c4(66966, 66967) c5(66968, 66969, 66970, 66971, 66972, 66973, 66974, 66975, 66976, 66977, 66978, 66979) c6( ) c7(bL, bM, T, dD, yY, EQ, cz, lr, aj, v, j, eq, cd, J, cF, pN, hb, aT, jy, d, ff, ml, Ej, en, cm, bz, fq, abV, ac, cxB, adg); #c1(PTPRD) c2(NP_002830) c3(receptor-type tyrosine-protein phosphatase delta isoform 1 precursor) c4(132594, 132595) c5(132596, 132597, 132598, 132599, 132600, 132601, 132602, 132603, 132604, 132605, 132606, 132607, 132608, 132609, 132610, 132611, 132612, 132613, 132614, 132615) c6( ) c7(bL, bM, T, dD, yY, EQ, cz, lr, aj, v, j, eq, cd, J, cF, pN, hb, aT, jy, d, ff, mQ, Ej, en, cm, bz, fq, abV, ac, cxB, adg); #c1(PTPRD) c2(NP_569075) c3(receptor-type tyrosine-protein phosphatase delta isoform 2 precursor) c4(233630) c5(233631, 233632, 233633, 233634, 233635, 233636) c6( ) c7(bL, bM, T, dD, yY, EQ, cz, lr, aj, v, j, eq, cd, J, cF, pN, hb, aT, jy, d, ff, mQ, Ej, en, cm, bz, fq, abV, ac, cxB, adg); #c1(PTPRD) c2(NP_569076) c3(receptor-type tyrosine-protein phosphatase delta isoform 3 precursor) c4(233637) c5(233638, 233639, 233640, 233641, 233642, 233643) c6( ) c7(bL, bM, T, dD, yY, EQ, cz, lr, aj, v, j, eq, cd, J, cF, pN, hb, aT, jy, d, ff, mQ, Ej, en, cm, bz, fq, abV, ac, cxB, adg); #c1(PTPRD) c2(NP_569077) c3(receptor-type tyrosine-protein phosphatase delta isoform 4 precursor) c4(233644) c5(233645, 233646, 233647, 233648, 233649, 233650) c6( ) c7(bL, bM, T, dD, yY, EQ, cz, lr, aj, v, j, eq, cd, J, cF, pN, hb, aT, jy, d, ff, ml, Ej, en, cm, bz, fq, abV, ac, cxB, adg); #c1(PTPRD) c2(XP_006716902) c3(receptor-type tyrosine-protein phosphatase delta isoform X29) c4( ) c5(290539, 290540, 290541, 290542, 290543, 290544) c6( ) c7(bL, bM, T, dD, yY, EQ, cz, Ir, aj, v, j, eq, cd, J, cF, pN, hb, aT, jy, d, ff, mQ, Ej, en, cm, bz, fq, abV, ac, cxB, adg); #c1(PTPRD) c2(XP_011516296) c3(receptor-type tyrosine-protein phosphatase delta isoform X23) c4(309496, 309497) c5(309498, 309499, 309500, 309501, 309502, 309503) c6( ) c7(bL, bM, T, dD, yY, EQ, cz, lr, aj, v, j, eq, cd, J, cF, pN, hb, aT, jy, d, ff, ml, Ej, en, cm, bz, fq, abV, ac, cxB, adg); #c1(PTPRE) c2(NP_006495) c3(receptor-type tyrosine-protein phosphatase epsilon isoform 1 precursor) c4(165719, 165720) c5( ) c6( ) c7(z, bM, ey, j, mQ); #c1(PTPRE) c2(NP_569119) c3(receptor-type tyrosine-protein phosphatase epsilon isoform 2) c4(233664) c5(233665, 233666, 233667, 233668, 233669, 233670) c6( ) c7(z, bM, ey, j, mQ); #c1(PTPRE) c2(XP_011538295) c3(receptor-type tyrosine-protein phosphatase epsilon isoform X2) c4( ) c5(335970) c6( ) c7(z, bM, ey, j, mQ); #c1(PTPRE) c2(XP_011538297) c3(receptor-type tyrosine-protein phosphatase epsilon isoform X4) c4(335971) c5(335972, 335973, 335974, 335975, 335976, 335977) c6( ) c7(z, bM, ey, j, mQ); #c1(PTPRE) c2(XP_011538300) c3(receptor-type tyrosine-protein phosphatase epsilon isoform X7) c4( ) c5(335978, 335979, 335980, 335981, 335982, 335983, 335984, 335985, 335986, 335987, 335988, 335989, 335990, 335991, 335992, 335993) c6( ) c7(z, bM, By, j, mQ); #c1(PTPRG) c2(NP_002832) c3(receptor-type tyrosine-protein phosphatase gamma) c4(132616) c5(132617, 132618, 132619, 132620, 132621, 132622) c6( ) c7(bL, x, T, Lu, bug, BO, s, bE, aW, buk, aE, aT, r, aA, lo, bB, j, ci, bK, fr, bz); #c1(PTPRG) c2(XP_005265410) c3(receptor-type tyrosine-protein phosphatase gamma isoform X2) c4(279527) c5(279528, 279529, 279530, 279531, 279532, 279533) c6( ) c7(bL, x, T, Lu, bug, BO, s, bE, aW, buk, aE, aT, r, aA, lo, bB, j, ci, bk, fr, bz); #c1(PTPRH) c2(NP_001154912) c3(receptor-type tyrosine-protein phosphatase H isoform 2 precursor) c4(61220) c5(61221, 61222, 61223, 61224, 61225, 61226) c6( ) c7(AK, pi, Zf, bez, rS, dV, r, wR, CZ, q, C, Za, B); #c1(PTPRH) c2(NP_002833) c3(receptor-type tyrosine-protein phosphatase H isoform 1 precursor) c4(132623) c5(132624, 132625, 132626, 132627, 132628, 132629) c6( ) c7(AK, pi, Zf, bez, rS, dV, r, wR, CZ, q, C, Za, B); #c1(PTPRJ) c2(NP_001091973) c3(receptor-type tyrosine-protein phosphatase eta isoform 2 precursor) c4(39095) c5(39096, 39097, 39098, 39099, 39100, 39101, 39102, 39103, 39104, 39105, 39106) c6( ) c7(T, fM, ht, jh, cz, bB, fd, hy, ci, do, ai, q, eA, ip, av, hd, py, xE, pe, Db, aT, dz, Od, s, aW, ff, r, en, ik, nM, nB, bE, dX, k, y, bt); #c1(PTPRJ) c2(NP_002834) c3(receptor-type tyrosine-protein phosphatase eta isoform 1 precursor) c4(132630, 132631) c5(132632, 132633, 132634, 132635, 132636, 132637, 132638) c6( ) c7(T, fM, ht, jh, cz, bB, fd, hy, ci, Do, ai, q, eA, ip, av, hd, py, xE, pe, Db, aT, dz, Od, s, aW, ff, r, en, ik, nM, nB, bE, dX, k, y, bt); #c1(PTPRK) c2(NP_001129120) c3(receptor-type tyrosine-protein phosphatase kappa isoform a precursor) c4(50831) c5(50832, 50833, 50834, 50835, 50836, 50837) c6( ) c7(ck, T, aW, Y, aT, hp, ae, eA, ad, sj); #c1(PTPRK) c2(NP_001278910) c3(receptor-type tyrosine-protein phosphatase kappa isoform c precursor) c4(110888, 110889) c5(110890, 110891, 110892, 110893, 110894, 110895, 110896, 110897, 110898, 110899, 110900, 110901) c6( ) c7(ck, T, aW, Y, aT, hp, ae, eA, ad, sj); #c1(PTPRK) c2(NP_001278912) c3(receptor-type tyrosine-protein phosphatase kappa isoform e) c4(110902) c5(110903, 110904, 110905, 110906, 110907, 110908) c6( ) c7(ck, T, aW, Y, aT, hp, ae, eA, ad, sj); #c1(PTPRK) c2(NP_001278913) c3(receptor-type tyrosine-protein phosphatase kappa isoform f precursor) c4(110909) c5(110910, 110911, 110912, 110913, 110914, 110915) c6( ) c7(ck, T, aW, Y, aT, hp, ae, eA, ad, sj); #c1(PTPRK) c2(NP_002835) c3(receptor-type tyrosine-protein phosphatase kappa isoform b precursor) c4(132639) c5(132640, 132641, 132642, 132643, 132644, 132645) c6( ) c7(ck, T, aW, Y, aT, hp, ae, eA, ad, sj); #c1(PTPRK) c2(XP_011534317) c3(receptor-type tyrosine-protein phosphatase kappa isoform X2) c4(330684, 330685) c5(330686, 330687, 330688, 330689, 330690, 330691, 330692, 330693, 330694, 330695, 330696, 330697) c6( ) c7(ck, T, aW, Y, aT, hp, ae, eA, ad, sj); #c1(PTPRM) c2(NP_001098714) c3(receptor-type tyrosine-protein phosphatase mu isoform 1 precursor) c4(41524, 41525) c5(41526, 41527, 41528, 41529, 41530, 41531, 41532, 41533, 41534, 41535, 41536, 41537) c6( ) c7(bL, T, aW, eZ, aT, V, ad); #c1(PTPRM) c2(NP_002836) c3(receptor-type tyrosine-protein phosphatase mu isoform 2 precursor) c4(132646, 132647) c5(132648, 132649, 132650, 132651, 132652, 132653, 132654, 132655, 132656, 132657, 132658, 132659) c6( ) c7(bL, T, aW, eZ, aT, V, ad); #c1(PTPRM) c2(XP_011524018) c3(receptor-type tyrosine-protein phosphatase mu isoform X11) c4(318537) c5(318538, 318539, 318540, 318541, 318542, 318543) c6( ) c7(bL, T, aW, eZ, aT, V, ad); #c1(PTPRN) c2(NP_001186692) c3(receptor-type tyrosine-protein phosphatase-like N isoform 2 precursor) c4(78353) c5(78354, 78355, 78356, 78357, 78358, 78359, 78360, 78361, 78362, 78363) c6( ) c7(bL, gx, mj, A, Hm, Mz, aj, mf, dG, hK, V, dF, J); #c1(PTPRN) c2(NP_001186693) c3(receptor-type tyrosine-protein phosphatase-like N isoform 3) c4(78364) c5(78365, 78366, 78367, 78368, 78369, 78370) c6( ) c7(bL, gx, mj, A, Hm, Mz, aj, mf, dG, hK, V, dF, J); #c1(PTPRN) c2(NP_002837) c3(receptor-type tyrosine-protein phosphatase-like N isoform 1 precursor) c4(132660, 132661) c5(132662, 132663, 132664, 132665, 132666, 132667, 132668) c6( ) c7(bL, gx, mj, A, Hm, Mz, aj, mf, dG, hK, V, dF, J); #c1(PTPRN) c2(XP_011509868) c3(receptor-type tyrosine-protein phosphatase-like N isoform X1) c4( ) c5(300468, 300469, 300470, 300471, 300472) c6( ) c7(bL, gx, mj, A, Hm, Mz, aj, mf, dG, hK, V, dF, J); #c1(PTPRN2) c2(NP_002838) c3(receptor-type tyrosine-protein phosphatase N2 isoform 1 precursor) c4(132669) c5(132670, 132671, 132672, 132673, 132674, 132675) c6( ) c7(bL, mj, A, ac, aT, Mz, aj, mf, dy, bX, he, J); #c1(PTPRN2) c2(NP_570857) c3(receptor-type tyrosine-protein phosphatase N2 isoform 2 precursor) c4(234345) c5(234346, 234347, 234348, 234349, 234350, 234351) c6( ) c7(bL, mj, A, ac, aT, Mz, aj, mf, dy, bX, he, J); #c1(PTPRN2) c2(NP_570858) c3(receptor-type tyrosine-protein phosphatase N2 isoform 3 precursor) c4(234352) c5(234353, 234354, 234355, 234356, 234357, 234358) c6( ) c7(bL, mj, A, ac, aT, Mz, aj, mf, dy, bX, he, J); #c1(PTPRO) c2(NP_002839) c3(receptor-type tyrosine-protein phosphatase O isoform b precursor) c4(132676, 132677) c5(132678, 132679, 132680, 132681, 132682, 132683, 132684, 132685) c6( ) c7(T, fM, jh, cz, ah, uE, x, av, kH, aT, bxg, iU, aW, Y, hl, aq, iR, fq, cxC, ck, sk, cj, it, B); #c1(PTPRO) c2(NP_109592) c3(receptor-type tyrosine-protein phosphatase 0 isoform a precursor) c4(219743) c5(219744, 219745, 219746, 219747, 219748, 219749, 219750, 219751) c6( ) c7(T, fM, jh, cz, ah, uE, x, av, kH, aT, bxg, iU, aW, Y, hl, aq, iR, fq, cxC, ck, sk, cj, it, B); #c1(PTPRO) c2(NP_109594) c3(receptor-type tyrosine-protein phosphatase 0 isoform c) c4( ) c5(219752, 219753, 219754, 219755, 219756, 219757, 219758, 219759) c6( ) c7(T, fM, jh, cz, ah, uE, x, av, kH, aT, bxg, iU, aW, Y, hl, aq, iR, fq, cxC, ck, sk, cj, it, B); #c1(PTPRO) c2(NP_109595) c3(receptor-type tyrosine-protein phosphatase 0 isoform d) c4( ) c5(219760, 219761, 219762, 219763, 219764, 219765, 219766, 219767) c6( ) c7(T, fM, jh, cz, ah, uE, x, av, kH, aT, bxg, iU, aW, Y, hl, aq, iR, fq, cxC, ck, sk, cj, it, B); #c1(PTPRO) c2(XP_011519094) c3(receptor-type tyrosine-protein phosphatase 0 isoform X1) c4(313687) c5( ) c6( ) c7(T, fM, jh, cz, ah, uE, x, av, kH, aT, bxg, iU, aW, Y, hl, aq, iR, fq, cxC, ck, sk, cj, it, B); #c1(PTPRQ) c2(NP_001138498) c3(phosphatidylinositol phosphatase PTPRQ precursor) c4(56898) c5(56899, 56900, 56901, 56902, 56903, 56904) c6( ) c7(Bh, bK, cxD, EQ); #c1(PTPRR) c2(NP_001193944) c3(receptor-type tyrosine-protein phosphatase R isoform 3) c4(83291, 83292, 83293) c5(83294, 83295, 83296, 83297, 83298, 83299) c6( ) c7(Bh, Be, an); #c1(PTPRR) c2(NP_001193945) c3(receptor-type tyrosine-protein phosphatase R isoform 4) c4(83300) c5(83301, 83302, 83303, 83304, 83305, 83306, 83307, 83308, 83309) c6( ) c7(Bh, Be, an); #c1(PTPRR) c2(NP_002840) c3(receptor-type tyrosine-protein phosphatase R isoform 1 precursor) c4(132686) c5(132687, 132688, 132689, 132690, 132691, 132692) c6( ) c7(Bh, Be, an); #c1(PTPRR) c2(NP_570897) c3(receptor-type tyrosine-protein phosphatase R isoform 2) c4( ) c5(234359, 234360, 234361, 234362, 234363, 234364, 234365, 234366, 234367, 234368, 234369, 234370, 234371) c6( ) c7(Bh, Be, an); #c1(PTPRS) c2(NP_002841) c3(receptor-type tyrosine-protein phosphatase S isoform 1 precursor) c4(132693, 132694, 132695) c5(132696, 132697, 132698, 132699, 132700, 132701, 132702, 132703, 132704, 132705, 132706, 132707, 132708, 132709, 132710, 132711, 132712, 132713) c6( ) c7(bL, ji, iV); #c1(PTPRS) c2(NP_570923) c3(receptor-type tyrosine-protein phosphatase S isoform 3 precursor) c4(234408) c5(234409, 234410, 234411, 234412, 234413, 234414) c6( ) c7(bL, ji, lV); #c1(PTPRS) c2(NP_570924) c3(receptor-type tyrosine-protein phosphatase S isoform 2 precursor) c4(234415) c5(234416, 234417, 234418, 234419, 234420, 234421) c6( ) c7(bL, ji, iV); #c1(PTPRS) c2(NP_570925) c3(receptor-type tyrosine-protein phosphatase S isoform 4 precursor) c4(234422) c5(234423, 234424, 234425, 234426, 234427, 234428) c6( ) c7(bL, ji, V); #c1(PTPRS) c2(XP_006722875) c3(receptor-type tyrosine-protein phosphatase S isoform X6) c4(294899) c5(294900, 294901, 294902, 294903, 294904, 294905) c6( ) c7(bL, ji, iV); #c1(PTPRS) c2(XP_006722877) c3(receptor-type tyrosine-protein phosphatase S isoform X7) c4(294906) c5(294907, 294908, 294909, 294910, 294911, 294912) c6( ) c7(bL, ji, iV); #c1(PTPRS) c2(XP_006722881) c3(receptor-type tyrosine-protein phosphatase S isoform X11) c4(294913) c5(294914, 294915, 294916, 294917, 294918, 294919) c6( ) c7(bL, ji, iV); #c1(PTPRS) c2(XP_011526459) c3(receptor-type tyrosine-protein phosphatase S isoform X15) c4(320761) c5(320762, 320763, 320764, 320765, 320766, 320767) c6( ) c7(bL, ji, iV); #c1(PTPRT) c2(NP_008981) c3(receptor-type tyrosine-protein phosphatase T isoform 2 precursor) c4(170033, 170034) c5(170035, 170036, 170037, 170038, 170039, 170040, 170041, 170042, 170043, 170044, 170045, 170046) c6( ) c7(bL, T, aC, bB, Wb, aj, fr, yV, eJ, eA, J, ad, ji, ae, d, bug, eZ, as, cm, bE, dy, y); #c1(PTPRT) c2(NP_573400) c3(receptor-type tyrosine-protein phosphatase T isoform 1 precursor) c4(234483, 234484) c5(234485, 234486, 234487, 234488, 234489, 234490, 234491, 234492, 234493, 234494, 234495, 234496) c6( ) c7(bL, T, aC, bB, Wb, aj, fr, yV, eJ, eA, J, ad, ji, ae, d, bug, eZ, as, cm, bE, dy, y); #c1 (PTPRT) c2(XP_011526815) c3(receptor-type tyrosine-protein phosphatase T isoform X3) c4(321115) c5(321116, 321117, 321118, 321119, 321120, 321121) c6( ) c7(bL, T, aC, bB, Wb, aj, fr, yV, eJ, eA, J, ad, ji, ae, d, bug, eZ, as, cm, bE, dy, y); #c1(PTPRT) c2(XP_011526816) c3(receptor-type tyrosine-protein phosphatase T isoform X4) c4(321122) c5(321123, 321124, 321125, 321126, 321127, 321128) c6( ) c7(bL, T, aC, bB, Wb, aj, fr, yV, eJ, eA, J, ad, ji, ae, d, bug, eZ, as, cm, bE, dy, y); #c1(PTPRT) c2(XP_011526817) c3(receptor-type tyrosine-protein phosphatase T isoform X5) c4(321129) c5(321130, 321131, 321132, 321133, 321134, 321135) c6( ) c7(bL, T, aC, bB, Wb, aj, fr, yV, eJ, eA, J, ad, ji, ae, d, bug, eZ, as, cm, bE, dy, y); #c1(PTPRZ1) c2(NP_001193768) c3(receptor-type tyrosine-protein phosphatase zeta isoform 3 precursor) c4(82558) c5(82559, 82560, 82561, 82562, 82563, 82564) c6( ) c7(T, wi, aA, cg, fr, il, V, Fc, eA, aR, ft, pX, aT, d, eH, r, as, eF, Hv, bN, fT, c, cB); #c1(PTPRZ1) c2(NP_002842) c3(receptor-type tyrosine-protein phosphatase zeta isoform 1 precursor) c4(132714) c5(132715, 132716, 132717, 132718, 132719, 132720, 132721) c6( ) c7(T, wi, aA, cg, fr, il, V, Fc, eA, aR, ft, pX, aT, d, eH, r, as, eF, Hv, bN, fT, c, cB); #c1(PTRF) c2(NP_036364) c3(polymerase 1 and transcript release factor) c4(173246, 173247) c5(173248, 173249, 173250, 173251, 173252, 173253, 173254, 173255, 173256, 173257, 173258, 173259) c6( ) c7(T, d, eM, iU, QJ, cxE, xs, jN, oj, v, PQ, qN, iV, ae, GR, ad, mu); #c1(PTRH1) c2(NP_001002913) c3(probable peptidyl-tRNA hydrolase) c4(19752) c5(19753, 19754, 19755, 19756, 19757, 19758, 19759, 19760, 19761, 19762) c6( ) c7(bL, T, zp, AB, eR, rV, pY, Ay, adi, amC, j, cxc, nA, cxa, ml, aY, pd, JW, xt, xx, fU, acX, ahy, amB, TI, bv, aE, zw, xW, ari, aT, yu, rD, bta, K, bsR, OM, aAG, zg, he, EL, yh, ars, aAL, bA, de, bDT, azh, Jg, aAl, s, iU, aW, OX, vj, eH, Lm, nh, dq, aq, eF, HI, bz, Vm, dr, wW, EJ, bRK, pv, YE, yl, TG, fP, hj, pg, iS, QF, aYS, azg, aAQ, B, aAD); #c1(PTRH2) c2(NP_057161) c3(peptidyl-tRNA hydrolase 2, mitochondrial precursor) c4( ) c5(189215, 189216, 189217, 189218, 189219, 189220, 189221, 189222, 189223) c6( ) c7(T, ft, aW, sw, ff, aT, en, it, ad); #c1(PTRHD1) c2(NP_001013685) c3(putative peptidyl-tRNA hydrolase PTRHD1) c4(25709) c5(25710, 25711, 25712, 25713, 25714, 25715) c6( ) c7(bk); #c1(PTS) c2(NP_000308) c3(6-pyruvoyl tetrahydrobiopterin synthase) c4(6821, 6822) c5(6823, 6824, 6825, 6826, 6827, 6828, 6829, 6830, 6831, 6832, 6833, 6834, 6835, 6836, 6837, 6838, 6839, 6840, 6841, 6842, 6843, 6844, 6845, 6846, 6847, 6848, 6849, 6850, 6851, 6852, 6853, 6854, 6855, 6856, 6857, 6858, 6859, 6860, 6861, 6862, 6863, 6864, 6865, 6866, 6867, 6868, 6869, 6870, 6871, 6872, 6873, 6874, 6875, 6876, 6877, 6878, 6879, 6880, 6881, 6882, 6883, 6884, 6885, 6886, 6887, 6888, 6889, 6890, 6891, 6892, 6893, 6894, 6895, 6896, 6897, 6898, 6899, 6900, 6901, 6902, 6903, 6904, 6905, 6906, 6907, 6908, 6909, 6910, 6911, 6912, 6913, 6914, 6915, 6916, 6917) c6(6918, 6919, 6920) c7(bfb, Wz, aU, dq, aq, acU, Wm, PJ, cxF, B, bN); #c1(PTTG1) c2(NP_001269312) c3(securin) c4(101067) c5(101068, 101069, 101070, 101071, 101072, 101073, 101074, 101075, 101076, 101077, 101078, 101079, 101080, 101081) c6( ) c7(T, aC, lT, ht, fN, fd, bB, n, aj, ci, q, J, ad, eA, wD, cF, hd, py, cc, aT, iv, ey, ae, af, yo, s, vT, qt, asP, Y, aW, ff, iw, cr, r, ks, aq, eZ, kp, en, hf, bu, t, g, bz, cm, op, iR, eM, Jr, nB, bE, vA, gX, gZ, hR, nw, aan, it, B); #c1(PTTG11P) c2(NP_001273751) c3(pituitary tumor-transforming gene 1 protein-interacting protein isoform 2 precursor) c4(105767) c5(105768, 105769, 105770, 105771, 105772, 105773) c6( ) c7(T, hd, s, nB, aW, eH, aT, hR, n, hz, eF); #c1(PTTG11P) c2(NP_004330) c3(pituitary tumor-transforming gene 1 protein-interacting protein isoform 1 precursor) c4(145213) c5(145214, 145215, 145216, 145217, 145218, 145219, 145220) c6( ) c7(T, hd, s, nB, aW, eH, aT, hR, n, hz, eF); #c1(PTTG2) c2(NP_006598) c3(securin-2) c4(166528) c5(166529, 166530, 166531, 166532, 166533, 166534) c6( ) c7(iR, aq); #c1(PTX3) c2(NP_002843) c3(pentraxin-related protein PTX3 precursor) c4(132722) c5(132723, 132724, 132725, 132726, 132727, 132728) c6( ) c7(oO, T, jv, dD, eR, fR, ea, fG, Hc, aGu, e, aw, wo, cR, eX, cl, v, eq, C, aBl, cd, o, ai, eA, azY, rS, aT, eV, ey, K, lQ, ao, qE, bzH, hm, yo, aiV, aW, Y, cr, yq, pm, uC, pb, LB, D, kT, rR, pc, y, ig, au, aiM, at, wq, wR, bas, aS, yH, dy, EY, hh, Pk, ei, aDj); #c1(PUF60) c2(NP_001129505) c3(poly(U)-binding-splicing factor PUF60 isoform c) c4( ) c5(51766, 51767, 51768, 51769, 51770) c6( ) c7(ov, T, ib, cxG, nO, r, bD, q); #c1(PUF60) c2(NP_001258026) c3(poly(U)-binding-splicing factor PUF60 isoform e) c4(95912) c5( ) c6( ) c7(ov, T, ib, cxG, nO, r, bD, q); #c1(PUF60) c2(NP_001258028) c3(poly(U)-binding-splicing factor PUF60 isoform g) c4(95913) c5( ) c6( ) c7(ov, T, ib, cxG, nO, r, bD, q); #c1 (PUF60) c2(NP_001258029) c3(poly(U)-binding-splicing factor PUF60 isoform h) c4( ) c5(95914, 95915, 95916, 95917, 95918, 95919, 95920, 95921) c6( ) c7(ov, T, ib, cxG, no, r, bD, q); #c1(PUF60) c2(NP_055096) c3(poly(U)-binding-splicing factor PUF60 isoform b) c4(178898) c5(178899, 178900, 178901, 178902, 178903, 178904, 178905, 178906, 178907) c6( ) c7(ov, T, ib, cxG, n, r, bD, q); #c1(PUF60) c2(NP_510965) c3(poly(U)-binding-splicing factor PUF60 isoform a) c4(231083) c5(231084, 231085, 231086, 231087, 231088, 231089, 231090, 231091, 231092) c6( ) c7(ov, T, ib, cxG, nO, r, bD, q); #c1(PUF60) c2(XP_011515231) c3(poly(U)-binding-splicing factor PUF60 isoform X1) c4( ) c5(308046) c6( ) c7(ov, T, ib, cxG, no, r, bD, q); #c1(PUF60) c2(XP_011515232) c3(poly(U)-binding-splicing factor PUF60 isoform X2) c4( ) c5(308047) c6( ) c7(ov, T, ib, cxG, nO, r, bD, q); #c1(PUM2) c2(NP_001269681) c3(pumilio homolog 2 isoform 2) c4( ) c5(102531, 102532, 102533, 102534, 102535) c6( ) c7(vH, gU); #c1(PUM2) c2(NP_056132) c3(pumilio homolog 2 isoform 1) c4(185850, 185851) c5(185852, 185853, 185854, 185855, 185856, 185857, 185858, 185859, 185860, 185861, 185862) c6( ) c7(vH, gU); #c1(PUM2) c2(XP_005262666) c3(pumilio homolog 2 isoform X4) c4(278031) c5(278032, 278033, 278034, 278035, 278036, 278037) c6( ) c7(vH, gU); #c1(PUM2) c2(XP_006712037) c3(pumilio homolog 2 isoform X2) c4(286354) c5(286355, 286356, 286357, 286358, 286359, 286360, 286361, 286362, 286363, 286364, 286365) c6( ) c7(vH, gU); #c1(PUM2) c2(XP_011531026) c3(pumilio homolog 2 isoform X6) c4(326987, 326988) c5( ) c6( ) c7(vH, gU); #c1(PUM2) c2(XP_011531027) c3(pumilio homolog 2 isoform X7) c4( ) c5(326989, 326990, 326991, 326992, 326993, 326994, 326995, 326996) c6( ) c7(vH, gU); #c1(PURA) c2(NP_005850) c3(transcriptional activator protein Pur-alpha) c4(159867) c5(159868, 159869, 159870, 159871, 159872, 159873) c6( ) c7(aj, bw, bt, af, J, ad); #c1(PURB) c2(NP_150093) c3(transcriptional activator protein Pur-beta) c4(227746) c5(227747, 227748, 227749, 227750, 227751, 227752) c6( ) c7(bw, bt, cd); #c1(PUS1) c2(NP_001002019) c3(tRNA pseudouridine synthase A, mitochondrial isoform 2) c4(19349, 19350, 19351) c5(19352, 19353, 19354, 19355, 19356, 19357, 19358, 19359, 19360, 19361, 19362, 19363, 19364, 19365, 19366, 19367, 19368, 19369) c6( ) c7(kA, aNG, cxH, oj, aA); #c1(PUS1) c2(NP_079491) c3(tRNA pseudouridine synthase A, mitochondrial isoform 1) c4(218341) c5(218342, 218343, 218344, 218345, 218346, 218347, 218348, 218349, 218350, 218351) c6( ) c7(kA, aNG, cxH, oj, aA); #c1(PUS1) c2(XP_011537071) c3(tRNA pseudouridine synthase A, mitochondrial isoform X2) c4( ) c5(334851) c6( ) c7(kA, aNG, cxH, oj, aA); #c1(PUS10) c2(XP_011530879) c3(putative tRNA pseudouridine synthase Pus10 isoform X1) c4(326881, 326882) c5(326883, 326884, 326885, 326886, 326887, 326888, 326889, 326890, 326891, 326892, 326893, 326894, 326895, 326896, 326897, 326898) c6( ) c7(hp, ju, iV); #c1(PVALB) c2(XP_011545203) c3(parvalbumin alpha isoform X2) c4( ) c5(343989, 343990, 343991, 343992, 343993, 343994) c6( ) c7(dt, Uc, ha, bO, lb, bS, fr, ct, ci, V, Ef, aaF, kf, yT, cxl, vc, bK, K, h, an, afV, hC, Z, cm, bN, D, c, uJ, xg, ek); #c1(PVR) c2(NP_001129240) c3(poliovirus receptor isoform beta precursor) c4(51279) c5(51280, 51281, 51282, 51283, 51284, 51285) c6( ) c7(T, fR, aFc, pl, V, q, eA, dP, zT, iu, xl, aT, h, cxJ, d, s, iE, aW, r, lo, Ph, g, pm, cm, bz, vk, ck, apM, iT, bru, axV, X); #c1(PVR) c2(NP_001129241) c3(poliovirus receptor isoform gamma precursor) c4(51286) c5(51287, 51288, 51289, 51290, 51291, 51292) c6( ) c7(T, fR, aFc, pl, V, q, eA, dP, zT, iu, xl, aT, h, cxJ, d, s, iE, aW, r, lo, Ph, g, pm, cm, bz, vk, ck, apM, iT, bru, axV, X); #c1(PVR) c2(NP_001129242) c3(poliovirus receptor isoform delta precursor) c4(51293) c5(51294, 51295, 51296, 51297, 51298, 51299) c6( ) c7(T, fR, aFc, pl, V, q, eA, dP, zT, iu, xl, aT, h, cxJ, d, s, iE, aW, r, lo, Ph, g, pm, cm, bz, vk, ck, apM, iT, bru, axV, X); #c1(PVR) c2(NP_006496) c3(poliovirus receptor isoform alpha precursor) c4(165721) c5(165722, 165723, 165724, 165725, 165726, 165727) c6( ) c7(T, fR, aFc, pl, V, q, eA, dP, zT, iu, xl, aT, h, cxJ, d, s, iE, aW, r, lo, Ph, g, pm, cm, bz, vk, ck, apM, iT, bru, axV, X); #c1(PVRL1) c2(NP_002846) c3(nectin-1 isoform 1 precursor) c4(132747) c5(132748, 132749, 132750, 132751, 132752, 132753, 132754, 132755) c6( ) c7(T, fR, cxK, oe, aj, zU, sl, eA, aR, J, hX, dP, Na, eW, hd, xl, amJ, aT, Nc, lQ, aW, ff, cr, eZ, fe, alb, en, g, eE, cm, cxl, es, iO, arB, Pa, nB, xD, dc, wR, CZ, y); #c1(PVRL1) c2(NP_976030) c3(nectin-1 isoform 2 precursor) c4(262816) c5(262817, 262818, 262819, 262820, 262821, 262822) c6( ) c7(T, fR, cxK, oe, aj, zU, sl, eA, aR, J, hX, dP, Na, eW, hd, xl, amJ, aT, Nc, lQ, aW, ff, cr, eZ, fe, alb, en, g, eE, cm, cxL, es, iO, arB, Pa, nB, xD, dc, wR, CZ, y); #c1(PVRL1) c2(NP_976031) c3(nectin-1 isoform 3 precursor) c4(262823) c5(262824, 262825, 262826, 262827, 262828, 262829) c6( ) c7(T, fR, cxK, oe, aj, zU, sl, eA, aR, J, hX, dP, Na, eW, hd, xl, amJ, aT, Nc, lQ, aW, ff, cr, eZ, fe, alb, en, g, eE, cm, cxl, es, iO, arB, Pa, nB, xD, dc, wR, CZ, y); #c1(PVRL2) c2(NP_001036189) c3(nectin-2 isoform delta precursor) c4(34040) c5(34041, 34042, 34043, 34044, 34045, 34046) c6( ) c7(ck, zT, T, Na, fM, eW, af, xl, iT, e, Nc, j, m, eo, t, V, fK); #c1(PVRL2) c2(NP_002847) c3(nectin-2 isoform alpha precursor) c4(132756) c5(132757, 132758, 132759, 132760, 132761, 132762) c6( ) c7(ck, zT, T, Na, fM, eW, af, xl, iT, e, Nc, j, m, eo, t, V, fK); #c1(PVRL3) c2(NP_001230215) c3(nectin-3 isoform 2 precursor) c4(86124) c5(86125, 86126, 86127, 86128, 86129, 86130) c6( ) c7(dP, Hx, mP, aW, aT, hD, at); #c1(PVRL3) c2(NP_001230217) c3(nectin-3 isoform 3) c4( ) c5(86131, 86132, 86133, 86134, 86135, 86136) c6( ) c7(dP, Hx, mP, aW, aT, hD, at); #c1(PVRL3) c2(NP_056295) c3(nectin-3 isoform 1 precursor) c4(186998) c5(186999, 187000, 187001, 187002, 187003, 187004) c6( ) c7(dP, Hx, mP, aW, aT, hD, at); #c1(PVRL3) c2(XP_011510966) c3(nectin-3 isoform X4) c4(301428) c5( ) c6( ) c7(dP, Hx, mP, aW, aT, hD, at); #c1(PVRL4) c2(NP_112178) c3(nectin-4 precursor) c4(220639) c5(220640, 220641, 220642, 220643, 220644, 220645) c6( ) c7(dP, zT, T, eM, cxM, amJ, aW, ff, aT, ci, zU, en, g, CQ, bz, iu); #c1(PVRL4) c2(XP_005245565) c3(nectin-4 isoform X3) c4(266725) c5(266726, 266727, 266728, 266729, 266730, 266731) c6( ) c7(dP, zT, T, eM, cxM, amJ, aW, ff, aT, ci, zU, en, g, CQ, bz, iu); #c1(PWP2) c2(NP_005040) c3(periodic tryptophan protein 2 homolog) c4(152381) c5(152382, 152383, 152384, 152385, 152386, 152387) c6( ) c7(f); #c1(PWWP2B) c2(NP_001092107) c3(PWWP domain-containing protein 28 isoform 2) c4(39396) c5(39397, 39398, 39399, 39400, 39401, 39402) c6( ) c7(kl); #c1(PWWP2B) c2(NP_612508) c3(PWWP domain-containing protein 28 isoform 1) c4(236669) c5(236670, 236671, 236672, 236673, 236674, 236675) c6( ) c7(kl); #c1(PXDN) c2(NP_036425) c3(peroxidasin homolog precursor) c4(173753, 173754) c5(173755, 173756, 173757, 173758, 173759, 173760, 173761, 173762, 173763, 173764, 173765, 173766) c6( ) c7(ac, Y, bt, r, dy, bz, cdT, bbG, cxN, q); #c1(PXDNL) c2(NP_653252) c3(peroxidasin-like protein precursor) c4(239357, 239358) c5(239359, 239360, 239361, 239362, 239363, 239364, 239365, 239366, 239367, 239368, 239369, 239370, 239371, 239372, 239373, 239374) c6(239375, 239376, 239377, 239378) c7(akG, oe, gU, ND); #c1(PXK) c2(NP_001276025) c3(PX domain-containing protein kinase-like protein isoform 3) c4(107783) c5(107784, 107785, 107786, 107787, 107788, 107789) c6( ) c7(y, ey, pb, eZ); #c1(PXK) c2(NP_001276027) c3(PX domain-containing protein kinase-like protein isoform 4) c4(107790) c5(107791, 107792, 107793, 107794, 107795, 107796) c6( ) c7(y, ey, pb, eZ); #c1(PXK) c2(NP_001276028) c3(PX domain-containing protein kinase-like protein isoform 5) c4( ) c5(107797) c6( ) c7(y, ey, pb, eZ); #c1(PXK) c2(NP_001276030) c3(PX domain-containing protein kinase-like protein isoform 7) c4( ) c5(107798, 107799, 107800, 107801, 107802, 107803, 107804, 107805, 107806, 107807) c6( ) c7(y, ey, pb, eZ); #c1(PXK) c2(NP_060241) c3(PX domain-containing protein kinase-like protein isoform 1) c4(194930, 194931, 194932, 194933) c5(194934, 194935, 194936, 194937, 194938, 194939, 194940, 194941, 194942, 194943, 194944, 194945) c6( ) c7(y, ey, pb, eZ); #c1(PXK) c2(XP_011532170) c3(PX domain-containing protein kinase-like protein isoform X9) c4( ) c5(328216) c6( ) c7(y, ey, pb, eZ); #c1(PXMP2) c2(NP_061133) c3(peroxisomal membrane protein 2) c4(199435) c5(199436, 199437, 199438, 199439, 199440, 199441, 199442) c6( ) c7(ob, xF, bkY, csv, agU, ci, fu, bX, eA, no, csC, aT, bl, bU, csy, mJ, csB, aW, bT, eH, aFT, eF, cbm, Hp, bkZ, sk, ql, csw, mG, bee, afO); #c1(PXN) c2(NP_001074324) c3(paxillin isoform 2) c4(37096) c5( ) c6( ) c7(T, fM, aA, bB, fr, ci, q, ad, eA, av, iu, ft, lg, aT, By, dL, K, ae, eL, iU, aW, Y, az, eH, r, Ej, aF, g, eF, t, ik, bz, Ew, eM, bE, m, B); #c1(PXN) c2(NP_001230685) c3(paxillin isoform 3) c4(86568) c5( ) c6( ) c7(T, fM, aA, bB, fr, ci, q, ad, eA, av, iu, ft, lg, aT, By, dL, K, ae, eL, iU, aW, Y, az, eH, r, Ej, aF, g, eF, t, ik, bz, Ew, eM, bE, m, B); #c1(PXN) c2(NP_002850) c3(paxillin isoform 1) c4(132785) c5( ) c6( ) c7(T, fM, aA, bB, fr, ci, q, ad, eA, av, iu, ft, lg, aT, By, dL, K, ae, eL, iU, aW, Y, az, eH, r, Ej, aF, g, eF, t, ik, bz, Ew, eM, bE, m, B); #c1(PXN) c2(NP_079433) c3 (paxillin isoform 4) c4( ) c5(218109, 218110, 218111, 218112, 218113, 218114, 218115, 218116, 218117, 218118, 218119) c6( ) c7(T, fM, aA, bB, fr, ci, q, ad, eA, av, iu, ft, lg, aT, By, dL, K, ae, eL, iU, aW, Y, az, eH, r, Ej, aF, g, eF, t, ik, bz, Ew, eM, bE, m, B); #c1(PXN) c2(XP_005253970) c3 (paxillin isoform X16) c4( ) c5(272810, 272811, 272812, 272813, 272814, 272815) c6( ) c7(T, fM, aA, bB, fr, ci, q, ad, eA, av, iu, ft, lg, aT, By, dL, K, ae, eL, iU, aW, Y, az, eH, r, Ej, aF, g, eF, t, ik, bz, Ew, eM, bE, m, B); #c1(PXN) c2(XP_005253972) c3(paxillin isoform X18) c4( ) c5(272816, 272817, 272818, 272819, 272820, 272821) c6( ) c7(T, fM, aA, bB, fr, ci, q, ad, eA, av, iu, ft, lg, aT, By, dL, K, ae, eL, iU, aW, Y, az, eH, r, Ej, aF, g, eF, t, iK, bz, Ew, eM, bE, m, B); #c1(PXN) c2(XP_005253973) c3(paxillin isoform X19) c4(272822, 272823) c5(272824, 272825, 272826, 272827, 272828, 272829, 272830, 272831) c6( ) c7(T, fM, aA, bB, fr, ci, q, ad, eA, av, iu, ft, lg, aT, By, dL, K, ae, eL, iU, aW, Y, az, eH, r, Ej, aF, g, eF, t, ik, bz, Ew, eM, bE, m, B); #c1(PXN) c2(XP_005253974) c3(paxillin isoform X20) c4(272832) c5(272833, 272834, 272835, 272836, 272837, 272838) c6( ) c7(T, fM, aA, bB, fr, ci, q, ad, eA, av, iu, ft, lg, aT, By, dL, K, ae, eL, iU, aW, Y, az, eH, r, Ej, aF, g, eF, t, iK, bz, Ew, eM, bE, m, B); #c1(PXN) c2(XP_006719594) c3(paxillin isoform X1) c4(292662) c5( ) c6( ) c7(T, fM, aA, bB, fr, ci, q, ad, eA, av, iu, ft, lg, aT, By, dL, K, ae, eL, iU, aW, Y, az, eH, r, Ej, aF, g, eF, t, iK, bz, Ew, eM, bE, m, B); #c1(PXN) c2(XP_011536927) c3(paxillin isoform X12) c4( ) c5(334592) c6( ) c7(T, fM, aA, bB, fr, ci, q, ad, eA, av, iu, ft, lg, aT, By, dL, K, ae, eL, iU, aW, Y, az, eH, r, Ej, aF, g, eF, t, iK, bz, Ew, eM, bE, m, B); #c1(PXN) c2(XP_011536929) c3(paxillin isoform X21) c4( ) c5(334593, 334594, 334595, 334596, 334597) c6( ) c7(T, fM, aA, bB, fr, ci, q, ad, eA, av, iu, ft, lg, aT, By, dL, K, ae, eL, iU, aW, Y, az, eH, r, Ej, aF, g, eF, t, ik, bz, Ew, eM, bE, m, B); #c1(PXT1) c2(XP_011512702) c3(peroxisomal testis-specific protein 1 isoform X2) c4(303739, 303740, 303741) c5(303742) c6( ) c7(gU); #c1(PXT1) c2(XP_011512703) c3(peroxisomal testis-specific protein 1 isoform X1) c4( ) c5(303743, 303744, 303745, 303746, 303747, 303748, 303749, 303750, 303751, 303752, 303753, 303754, 303755, 303756, 303757, 303758, 303759) c6( ) c7(gU); #c1(PYCARD) c2(NP_037390) c3 (apoptosis-associated speck-like protein containing a CARD isoform a) c4(175195, 175196) c5(175197, 175198, 175199, 175200, 175201, 175202, 175203, 175204, 175205, 175206, 175207, 175208) c6( ) c7(Dw, T, dD, QO, ea, if, al, aA, bB, n, btu, eq, ci, qN, q, ad, ai, eW, av, ft, hd, xE, jz, akU, ds, aoA, BJ, dV, aT, h, agV, K, ae, cp, sj, zL, d, s, up, aW, Y, AQ, ih, ff, r, hQ, aq, lo, aF, en, g, ajz, t, as, bz, cm, ck, gk, By, nM, eM, nB, bE, wE, dy, m, lS, hh, y, B, cn); #c1(PYCARD) c2(NP_660183) c3(apoptosis-associated speck-like protein containing a CARD isoform b) c4(240468) c5(240469, 240470, 240471, 240472, 240473, 240474) c6( ) c7(Dw, T, dD, oO, ea, if, al, aA, bB, n, btu, eq, ci, qN, q, ad, ai, eW, av, ft, hd, xE, jz, akU, ds, aoA, BJ, dV, aT, h, agV, K, ae, cp, sj, zL, d, s, up, aW, Y, AQ, ih, ff, r, hl, aq, lo, aF, en, g, ajz, t, as, bz, cm, ck, gk, Qy, nM, eM, nB, bE, wE, dy, m, lS, hh, y, B, cn); #c1(PYCR1) c2(NP_001269208) c3(pyrroline-5-carboxylate reductase 1, mitochondrial isoform 3) c4(100779) c5(100780, 100781, 100782, 100783, 100784) c6( ) c7(RR, iT, cxO, Uj, lP, rC, CQ, nA, cxl, cxP, ae, ad); #c1(PYCR1) c2(NP_001269210) c3 (pyrroline-5-carboxylate reductase 1, mitochondrial isoform 5) c4(100785) c5(100786, 100787, 100788, 100789, 100790, 100791, 100792, 100793) c6( ) c7(RR, iT, cxO, Uj, lP, rC, CQ, nA, cxl, cxP, ae, ad); #c1(PYCR1) c2(NP_722546) c3(pyrroline-5-carboxylate reductase 1, mitochondrial isoform 2) c4(246814) c5(246815, 246816, 246817, 246818, 246819, 246820) c6( ) c7(RR, iT, cxO, Uj, lP, rC, CQ, nA, cxQ, cxP, ae, ad); #c1(PYCR1) c2(XP_011521886) c3(pyrroline-5-carboxylate reductase 1, mitochondrial isoform X1) c4(316492, 316493) c5(316494, 316495, 316496, 316497, 316498) c6( ) c7(RR, iT, cxO, Uj, lP, rC, CQ, nA, cxl, cxP, ae, ad); #c1(PYCR1) c2(XP_011521887) c3 (pyrroline-5-carboxylate reductase 1, mitochondrial isoform X2) c4( ) c5(316499, 316500, 316501, 316502, 316503, 316504) c6( ) c7(RR, iT, cxO, Uj, lP, rC, CQ, nA, cxQ, cxP, ae, ad); #c1(PYDC1) c2(NP_690865) c3(pyrin domain-containing protein 1) c4(245012) c5(245013, 245014, 245015, 245016, 245017, 245018) c6( ) c7(T, aet, awB, ciV, K, Eh, adz); #c1(PYDC2) c2(NP_001076777) c3(pyrin domain-containing protein 2) c4(37534) c5(37535, 37536, 37537, 37538, 37539, 37540) c6( ) c7(ac, FO, fq, FP, aC); #c1(PYGB) c2(NP_002853) c3(glycogen phosphorylase, brain form) c4(132793, 132794) c5(132795, 132796, 132797, 132798, 132799, 132800, 132801, 132802, 132803, 132804, 132805, 132806) c6( ) c7(ac, eM, qK, j, g, af); #c1(PYGL) c2(NP_001157412) c3(glycogen phosphorylase, liver form isoform 2) c4(62164) c5(62165, 62166, 62167, 62168, 62169, 62170) c6( ) c7(byp, A, UT, vX, Bw, B); #c1(PYGL) c2(NP_002854) c3(glycogen phosphorylase, liver form isoform 1) c4(132807) c5(132808, 132809, 132810, 132811, 132812, 132813, 132814, 132815, 132816, 132817, 132818, 132819, 132820, 132821, 132822, 132823, 132824, 132825, 132826, 132827, 132828, 132829, 132830, 132831, 132832, 132833, 132834, 132835, 132836, 132837, 132838, 132839, 132840, 132841, 132842, 132843, 132844, 132845, 132846, 132847, 132848, 132849, 132850, 132851, 132852, 132853, 132854, 132855, 132856, 132857, 132858, 132859, 132860, 132861, 132862, 132863, 132864, 132865, 132866, 132867, 132868, 132869, 132870, 132871, 132872, 132873, 132874, 132875, 132876, 132877, 132878, 132879, 132880, 132881, 132882, 132883, 132884, 132885, 132886, 132887, 132888, 132889, 132890, 132891, 132892, 132893, 132894, 132895, 132896, 132897, 132898, 132899, 132900) c6(132901) c7(byp, A, UT, vX, Bw, B); #c1(PYGM) c2(NP_001158188) c3(glycogen phosphorylase, muscle form isoform 2) c4(63301, 63302) c5(63303, 63304, 63305, 63306, 63307, 63308) c6( ) c7(nD, mj, pY, aW, vX, Bw, oj, aT, hj, aHs, dq, hR, j, ci, C, bq, hJ, uC, add); #c1(PYGM) c2(NP_005600) c3(glycogen phosphorylase, muscle form isoform 1) c4(157874, 157875) c5(157876, 157877, 157878, 157879, 157880, 157881, 157882, 157883, 157884, 157885, 157886, 157887, 157888, 157889) c6( ) c7(nD, mj, pY, aW, vX, Bw, oj, aT, hj, aHs, dq, hR, j, ci, C, bq, hJ, uC, add); #c1(PYG01) c2(NP_056432) c3(pygopus homolog 1) c4(187559) c5(187560, 187561, 187562, 187563, 187564, 187565) c6( ) c7(r); #c1(PYG01) c2(XP_011519748) c3(pygopus homolog 1 isoform X1) c4(314436) c5(314437, 314438, 314439, 314440, 314441, 314442) c6( ) c7(r); #c1(PYG02) c2(NP_612157) c3(pygopus homolog 2) c4(236113, 236114) c5(236115, 236116, 236117, 236118, 236119, 236120, 236121, 236122, 236123, 236124, 236125, 236126, 236127) c6( ) c7(T, d, aW, aT, ci, t, eA); #c1(PYHIN1) c2(NP_689714) c3(pyrin and HIN domain-containing protein I isoform alpha 1) c4(243660, 243661) c5(243662, 243663, 243664, 243665, 243666, 243667) c6( ) c7(aW, eZ, bM, aT); #c1(PYHIN1) c2(NP_945146) c3(pyrin and HIN domain-containing protein 1 isoform alpha 2) c4(260667) c5(260668, 260669, 260670, 260671, 260672, 260673) c6( ) c7(aW, eZ, bM, aT); #c1(PYHIN1) c2(NP_945147) c3(pyrin and HIN domain-containing protein 1 isoform beta 1) c4(260674) c5(260675, 260676, 260677, 260678, 260679, 260680) c6( ) c7(aW, eZ, bM, aT); #c1(PYHIN1) c2(NP_945148) c3(pyrin and HIN domain-containing protein 1 isoform beta 2) c4(260681) c5(260682, 260683, 260684, 260685, 260686, 260687) c6( ) c7(aW, eZ, bM, aT); #c1(PYHIN1) c2(XP_005244987) c3(pyrin and HIN domain-containing protein 1 isoform X3) c4(266172) c5(266173, 266174, 266175, 266176, 266177, 266178, 266179, 266180, 266181, 266182, 266183, 266184) c6( ) c7(aW, eZ, bM, aT); #c1(PYROXD1) c2(NP_079130) c3(pyridine nucleotide-disulfide oxidoreductase domain-containing protein 1) c4(217185, 217186) c5(217187, 217188, 217189, 217190, 217191, 217192, 217193) c6( ) c7(nJ, vT); #c1(PYROXD1) c2(XP_006719216) c3(pyridine nucleotide-disulfide oxidoreductase domain-containing protein 1 isoform X1) c4( ) c5(292311, 292312, 292313, 292314, 292315) c6( ) c7(nJ, vT); #c1(PYY) c2(NP_004151) c3(peptide YY preproprotein) c4(143910) c5( ) c6( ) c7(bL, bF, T, iX, ev, Ji, qQ, aeS, al, yc, cz, jy, v, afE, qM, cxR, ei, af); #c1(PYY) c2(XP_011523337) c3(peptide YY isoform X1) c4(317877) c5(317878, 317879, 317880, 317881, 317882, 317883) c6( ) c7(bL, bF, T, iX, ev, Ji, qQ, aeS, aT, yc, cz, jy, v, afE, qM, cxR, ei, af); #c1(PZP) c2(NP_002855) c3(pregnancy zone protein precursor) c4(132902, 132903) c5(132904, 132905, 132906, 132907, 132908, 132909) c6( ) c7(af, fc); #c1(PZP) c2(XP_011519109) c3(pregnancy zone protein isoform X4) c4( ) c5(313711, 313712, 313713, 313714, 313715, 313716) c6( ) c7(af, fc); #c1(GARS) c2(NP_001259002) c3(glutamine-tRNA ligase isoform b) c4(96925) c5(96926, 96927, 96928, 96929, 96930, 96931) c6( ) c7(QK, ha, ad); #c1(GARS) c2(NP_005042) c3(glutamine-tRNA ligase isoform a) c4(152401) c5(152402, 152403, 152404, 152405, 152406, 152407) c6( ) c7(QK, ha, ad); #c1(QARS) c2(XP_011532275) c3(glutamine-tRNA ligase isoform X1) c4(328336) c5(328337, 328338, 328339, 328340, 328341, 328342) c6( ) c7(QK, ha, ad); #c1(QDPR) c2(NP_000311) c3(dihydropteridine reductase) c4(6935) c5(6936, 6937, 6938, 6939, 6940, 6941) c6( ) c7(bfb, b, i, lW, Ad, bcD, aU, cz, Xg, acU, bK, K, nA, bq, qj, bAk, cB, aR, bN, pC); #c1(QKI) c2(NP_001288014) c3(protein quaking isoform 5) c4(113418) c5(113419, 113420, 113421, 113422, 113423, 113424) c6( ) c7(T, y, dY, pN, aw, EQ, bO, bS, he, Ea, mF, t, ae, eA, ad, hq); #c1(QKI) c2(NP_006766) c3 (protein quaking isoform 1) c4(167882) c5(167883, 167884, 167885, 167886, 167887, 167888) c6( ) c7(T, y, dY, pN, aw, EQ, bO, bS, he, Ea, mF, t, ae, eA, ad, hq); #c1(QKI) c2(NP_996735) c3(protein quaking isoform 2) c4(264374) c5(264375, 264376, 264377, 264378, 264379, 264380) c6( ) c7(T, y, dY, pN, aw, EQ, bO, bS, he, Ea, mF, t, ae, eA, ad, hq); #c1(QKI) c2(NP_996736) c3 (protein quaking isoform 3) c4(264381) c5(264382, 264383, 264384, 264385, 264386, 264387) c6( ) c7(T, y, dY, pN, aw, EQ, bO, bS, he, Ea, mF, t, ae, eA, ad, hq); #c1(QKI) c2(NP_996737) c3(protein quaking isoform 4) c4(264388) c5(264389, 264390, 264391, 264392, 264393, 264394) c6( ) c7(T, y, dY, pN, aw, EQ, bO, bS, he, Ea, mF, t, ae, eA, ad, hq); #c1(QKI) c2(XP_011534563) c3 (protein quaking isoform X4) c4(331105) c5( ) c6( ) c7(T, y, dY, pN, aw, EQ, bO, bS, he, Ea, mF, t, ae, eA, ad, hq); #c1(QPCT) c2(NP_036545) c3(glutaminyl-peptide cyclotransferase precursor) c4(174314, 174315) c5(174316, 174317, 174318, 174319, 174320, 174321, 174322, 174323, 174324, 174325, 174326, 174327) c6( ) c7(T, wu, EQ, aA, n, j, ci, C, qj, fr, ber, q, BJ, hD, rD, bK, wm, hH, af, bA, A, UL, vT, NE, Y, fJ, ff, cr, r, eZ, aF, en, Pd, cl, t, g, eE, cm, dR, ck, yl, eM, eD, QT, cj, iS, zu, KY, y, pC); #c1(QPCTL) c2(NP_001156849) c3(glutaminyl-peptide cyclotransferase-like protein isoform 2) c4(62001) c5(62002, 62003, 62004, 62005, 62006, 62007) c6( ) c7(fq); #c1(QPCTL) c2(NP_060129) c3(glutaminyl-peptide cyclotransferase-like protein isoform 1) c4(194304) c5(194305, 194306, 194307, 194308, 194309, 194310, 194311, 194312) c6(194313, 194314) c7(fq); #c1(APRT) c2(NP_055113) c3(nicotinate-nucleotide pyrophosphorylase [carboxylating] precursor) c4( ) c5(179019, 179020, 179021, 179022, 179023, 179024, 179025, 179026, 179027, 179028) c6( ) c7(qJ, AN, AS); #c1(APRT) c2(XP_005255280) c3(nicotinate-nucleotide pyrophosphorylase [carboxylating] isoform X1) c4(273580) c5(273581) c6( ) c7(qJ, AN, AS); #c1(APRT) c2(XP_011544080) c3(nicotinate-nucleotide pyrophosphorylase [carboxylating] isoform X2) c4(343251) c5(343252, 343253, 343254, 343255, 343256, 343257) c6( ) c7(qJ, AN, AS); #c1(QRFP) c2(NP_937823) c3(orexigenic neuropeptide QRFP preproprotein) c4(259199) c5(259200, 259201, 259202, 259203, 259204, 259205, 259206, 259207) c6(259208, 259209) c7(v); #c1(QRFPR) c2(NP_937822) c3(pyroglutamylated RFamide peptide receptor) c4(259192) c5(259193, 259194, 259195, 259196, 259197, 259198) c6( ) c7(j); #c1(QRSLI) c2(NP_060762) c3(glutamyl-tRNA(Gln) amidotransferase subunit A, mitochondrial) c4(197657, 197658) c5(197659, 197660, 197661, 197662, 197663, 197664, 197665, 197666, 197667, 197668, 197669, 197670) c6( ) c7(T, ht, aA, bB, n, oY, v, j, cd, q, ad, er, dP, MS, bpp, aT, iY, K, j, hu, ae, A, iU, aW, OX, aSt, aF, BG, PU, t, f, bz, cm, bw, ck, gY, xO, bE, wR, m, Mn, bD, bt, B); #c1(QSOX1) c2(NP_001004128) c3(sulfhydryl oxidase 1 isoform b precursor) c4(20461) c5(20462, 20463, 20464, 20465, 20466, 20467, 20468, 20469) c6(20470, 20471) c7(G, aC, aE, aW, aT, kp, K, lB); #c1(QSOX1) c2(NP_002817) c3(sulfhydryl oxidase 1 isoform a precursor) c4(132445) c5(132446, 132447, 132448, 132449, 132450, 132451, 132452, 132453) c6(132454, 132455) c7(G, aC, aE, aW, aT, kp, K, lB); #c1(QSOX2) c2(NP_859052) c3(sulfhydryl oxidase 2 precursor) c4(255395, 255396) c5(255397, 255398, 255399, 255400, 255401, 255402, 255403, 255404, 255405, 255406, 255407, 255408) c6( ) c7(cm); #c1(QTRT1) c2(NP_112486) c3(queuine tRNA-ribosyltransferase) c4(221144, 221145) c5(221146, 221147, 221148, 221149, 221150, 221151, 221152, 221153, 221154, 221155, 221156, 221157) c6( ) c7(bF, gV, ar, av, iV, arA, hG, iS, fd, Kf, ey, ci, R, azr, g, as, qs, bz, y, aJM); #c1(R3HCC1) c2(NP_001129580) c3(R3H and coiled-coil domain-containing protein 1 isoform a) c4(51860, 51861) c5(51862, 51863, 51864, 51865, 51866, 51867, 51868, 51869, 51870, 51871) c6( ) c7(cxS, kl); #c1(R3HCC1) c2(NP_001288579) c3(R3H and coiled-coil domain-containing protein 1 isoform b) c4(113774, 113775, 113776) c5(113777, 113778, 113779, 113780, 113781, 113782, 113783, 113784, 113785, 113786, 113787, 113788, 113789, 113790, 113791, 113792, 113793, 113794, 113795, 113796, 113797, 113798, 113799, 113800) c6( ) c7(cxS, kl); #c1(R3HCC1L) c2(NP_001243548) c3(coiled-coil domain-containing protein R3HCC1L isoform 1) c4(91021) c5( ) c6( ) c7(Pg); #c1(R3HCC1L) c2(NP_001243549) c3(coiled-coil domain-containing protein R3HCC1L isoform 2) c4( ) c5(91022, 91023, 91024, 91025, 91026) c6( ) c7(Pg); #c1(R3HCC1L) c2(NP_001243550) c3(coiled-coil domain-containing protein R3HCC1L isoform 3) c4( ) c5(91027, 91028, 91029, 91030, 91031, 91032, 91033, 91034, 91035, 91036, 91037, 91038) c6( ) c7(Pg); #c1(R3HCC1L) c2(XP_011537941) c3(coiled-coil domain-containing protein R3HCC1L isoform X2) c4(335451) c5(335452) c6( ) c7(Pg); #c1(R3HCC1L) c2(XP_011537950) c3(coiled-coil domain-containing protein R3HCC1L isoform X1) c4(335453) c5(335454, 335455, 335456, 335457, 335458) c6( ) c7(Pg); #c1(R3HCC1L) c2(XP_011537951) c3(coiled-coil domain-containing protein R3HCC1L isoform X3) c4(335459, 335460) c5(335461, 335462, 335463, 335464, 335465, 335466, 335467, 335468, 335469, 335470, 335471) c6( ) c7(Pg); #c1(R3HDM1) c2(NP_001269728) c3(R3H domain-containing protein 1 isoform c) c4(102727) c5(102728, 102729, 102730, 102731, 102732, 102733) c6( ) c7(cm); #c1(R3HDM1) c2(NP_056176) c3(R3H domain-containing protein 1 isoform b) c4(186151) c5(186152, 186153, 186154, 186155, 186156, 186157) c6( ) c7(cm); #c1(R3HDM1) c2(XP_011509184) c3(R3H domain-containing protein 1 isoform X16) c4( ) c5(299800) c6( ) c7(cm); #c1(R3HDML) c2(NP_848586) c3(peptidase inhibitor R3HDML precursor) c4(253885) c5(253886, 253887, 253888, 253889, 253890, 253891, 253892, 253893, 253894) c6( ) c7(gf); #c1(RAB11A) c2(NP_001193765) c3(ras-related protein Rab-11A isoform 2) c4(82551) c5(82552, 82553, 82554, 82555, 82556, 82557) c6( ) c7(bpj, aW, ds, yk, ff, aT, NI, h, ci, K, en, hJ); #c1(RAB11A) c2(NP_004654) c3(ras-related protein Rab-11A isoform 1) c4(148541) c5(148542, 148543, 148544, 148545, 148546, 148547) c6( ) c7(bpj, aW, ds, yk, ff, aT, NI, h, ci, K, en, hJ); #c1(RAB11B) c2(NP_004209) c3(ras-related protein Rab-11B) c4(144328) c5(144329, 144330, 144331, 144332, 144333, 144334) c6( ) c7(h); #c1(RAB11FIP1) c2(NP_001002814) c3(rab11 family-interacting protein 1 isoform 3) c4(19615, 19616) c5(19617, 19618, 19619, 19620, 19621, 19622, 19623) c6( ) c7(h, aW, aT); #c1(RAB11FIP1) c2(NP_079427) c3(rab11 family-interacting protein 1 isoform 1) c4(218058, 218059) c5(218060, 218061, 218062, 218063, 218064, 218065, 218066, 218067, 218068, 218069, 218070, 218071, 218072, 218073, 218074, 218075, 218076, 218077) c6( ) c7(h, aW, aT); #c1(RAB11FIP2) c2(NP_055719) c3(rab11 family-interacting protein 2) c4(183037) c5(183038, 183039, 183040, 183041, 183042, 183043) c6( ) c7(ey); #c1(RAB11FIP2) c2(XP_005269686) c3(rab11 family-interacting protein 2 isoform X1) c4(282713) c5(282714, 282715, 282716, 282717, 282718, 282719) c6( ) c7(ey); #c1(RAB11FIP3) c2(NP_001135744) c3(rab11 family-interacting protein 3 isoform 2) c4(52970, 52971, 52972) c5(52973, 52974, 52975, 52976, 52977, 52978, 52979, 52980, 52981, 52982, 52983, 52984, 52985, 52986, 52987, 52988, 52989, 52990) c6( ) c7(aW, TT, aT); #c1(RAB11FIP3) c2(NP_055515) c3(rab11 family-interacting protein 3 isoform 1) c4(181652) c5(181653, 181654, 181655, 181656, 181657, 181658) c6( ) c7(aW, TT, aT); #c1(RAB11FIP3) c2(XP_005255770) c3(rab11 family-interacting protein 3 isoform X1) c4(273865) c5(273866, 273867, 273868, 273869, 273870, 273871) c6( ) c7(aW, TT, aT); #c1(RAB11FIP3) c2(XP_005255775) c3(rab11 family-interacting protein 3 isoform X6) c4( ) c5(273872) c6( ) c7(aW, TT, aT); #c1(RAB11FIP4) c2(NP_116321) c3(rab11 family-interacting protein 4 isoform 1) c4(226134) c5(226135, 226136, 226137, 226138, 226139, 226140, 226141, 226142, 226143, 226144) c6( ) c7(cz); #c1(RAB11FIP5) c2(NP_056285) c3(rab11 family-interacting protein 5) c4(186936) c5(186937, 186938, 186939, 186940, 186941, 186942) c6( ) c7(bN); #c1(RAB12) c2(NP_001020471) c3(ras-related protein Rab-12) c4(28821) c5(28822, 28823, 28824, 28825, 28826, 28827) c6( ) c7(j); #c1(RAB14) c2(NP_057406) c3(ras-related protein Rab-14) c4(190575, 190576) c5(190577, 190578, 190579, 190580, 190581, 190582, 190583, 190584, 190585, 190586, 190587, 190588) c6( ) c7(h, [i, eM); #c1(RAB15) c2(NP_941959) c3(ras-related protein Rab-15) c4(260335) c5(260336, 260337, 260338, 260339, 260340, 260341) c6( ) c7(at, dL); #c1(RAB18) c2(NP_001243339) c3(ras-related protein Rab-18 isoform 2) c4(90431) c5(90432, 90433, 90434, 90435, 90436, 90437) c6( ) c7(cxV, yo, OF, cxU, s, cxT, jx, hR, aq, v, JW, B, hX); #c1(RAB18) c2(NP_001243341) c3(ras-related protein Rab-18 isoform 5) c4(90438) c5(90439, 90440, 90441, 90442, 90443, 90444) c6( ) c7(cxV, yo, OF, cxU, s, cxT, jx, hR, aq, v, JW, B, hX); #c1(RAB18) c2(NP_001243344) c3(ras-related protein Rab-18 isoform 4) c4(90450, 90451) c5(90452, 90453, 90454, 90455, 90456, 90457, 90458, 90459, 90460, 90461, 90462, 90463) c6( ) c7(cxV, yo, OF, cxU, s, cxT, jx, hR, aq, v, JW, B, hX); #c1(RAB18) c2(NP_067075) c3(ras-related protein Rab-18 isoform 1) c4(208829) c5(208830, 208831, 208832, 208833, 208834, 208835) c6( ) c7(cxV, yo, OF, cxU, s, cxT, jx, hR, aq, v, JW, B, hX); #c1(RAB1A) c2(NP_004152) c3(ras-related protein Rab-IA isoform 1) c4(143911, 143912) c5(143913, 143914, 143915, 143916, 143917, 143918, 143919, 143920, 143921, 143922, 143923, 143924) c6( ) c7(kf, ci, nz, ae, cd, ad); #c1(RAB1A) c2(NP_056358) c3(ras-related protein Rab-IA isoform 2) c4(187261, 187262) c5(187263, 187264, 187265, 187266, 187267, 187268, 187269, 187270, 187271, 187272, 187273, 187274) c6( ) c7(kf, ci, nz, ae, cd, ad); #c1(RAB1A) c2(XP_005264525) c3(ras-related protein Rab-IA isoform X2) c4(278990, 278991) c5(278992, 278993, 278994, 278995, 278996, 278997, 278998, 278999, 279000, 279001, 279002, 279003) c6( ) c7(kf, ci, nz, ae, cd, ad); #c1(RAB1B) c2(NP_112243) c3(ras-related protein Rab-IB) c4(221080, 221081, 221082) c5(221083, 221084, 221085, 221086, 221087, 221088, 221089, 221090, 221091, 221092, 221093, 221094, 221095, 221096, 221097, 221098, 221099, 221100) c6( ) c7(T, vT, aW, aT, j, K, cm, dR); #c1(RAB20) c2(NP_060287) c3(ras-related protein Rab-20) c4(195202) c5(195203, 195204, 195205, 195206, 195207, 195208) c6( ) c7(T, aC, aE, s, n, ci, g); #c1(RAB21) c2(NP_055814) c3(ras-related protein Rab-21) c4(183667) c5(183668, 183669, 183670, 183671, 183672, 183673) c6( ) c7(T); #c1(RAB22A) c2(NP_065724) c3(ras-related protein Rab-22A) c4(205063) c5(205064, 205065, 205066, 205067, 205068, 205069) c6( ) c7(aj); #c1(RAB23) c2(NP_001265595) c3(ras-related protein Rab-23) c4(98919) c5(98920, 98921, 98922, 98923, 98924, 98925, 98926, 98927, 98928, 98929) c6( ) c7(bDi, OS, aA, bDI, j, v, bz, aF, fq, B, zV, uQ); #c1(RAB24) c2(NP_570137) c3(ras-related protein Rab-24) c4(234032, 234033) c5(234034, 234035, 234036, 234037, 234038, 234039, 234040, 234041, 234042, 234043, 234044, 234045, 234046, 234047, 234048, 234049) c6( ) c7(KB); #c1(RAB25) c2(NP_065120) c3(ras-related protein Rab-25) c4(204046) c5(204047, 204048, 204049, 204050, 204051, 204052) c6( ) c7(cxW, T, sw, n, oH, Yc, aT, dL, K, an, eL, qt, aW, g, t, iK, bz, kP, bE, m, it, X); #c1(RAB27A) c2(NP_899059) c3(ras-related protein Rab-27A) c4(257913, 257914) c5(257915, 257916, 257917, 257918, 257919, 257920, 257921, 257922, 257923, 257924, 257925, 257926, 257927, 257928, 257929, 257930) c6( ) c7(cL, ci, pz, eA, aR, o, sl, dE, aT, dL, ey, eL, AT, lE, aW, Y, oV, ck, XF, km, nw, y, zA, aPG); #c1(RAB27B) c2(XP_005266790) c3(ras-related protein Rab-27B isoform XI) c4(280331) c5(280332, 280333, 280334, 280335, 280336, 280337) c6( ) c7(AT, lE, aW, aT, dL, el); #c1(RAB28) c2(NP_001017979) c3(ras-related protein Rab-28 isoform 1) c4(26717, 26718) c5(26719, 26720, 26721, 26722, 26723, 26724) c6( ) c7(nx, nC, fq, cxX); #c1(RAB28) c2(NP_001153073) c3(ras-related protein Rab-28 isoform 3) c4(60421) c5(60422, 60423, 60424, 60425, 60426, 60427) c6( ) c7(nx, nC, fq, cxX); #c1(RAB28) c2(NP_004240) c3(ras-related protein Rab-28 isoform 2) c4(144553) c5(144554, 144555, 144556, 144557, 144558, 144559) c6( ) c7(nx, nC, fq, cxX); #c1(RAB28) c2(XP_005248275) c3(ras-related protein Rab-28 isoform X4) c4( ) c5(268287, 268288, 268289, 268290, 268291) c6( ) c7(nx, nC, fq, cxX); #c1(RAB28) c2(XP_011512214) c3(ras-related protein Rab-28 isoform X3) c4( ) c5(302785) c6( ) c7(nx, nC, fq, cxX); #c1(RAB29) c2(NP_001129135) c3(ras-related protein Rab-7L1 isoform 2) c4(50880, 50881) c5(50882, 50883, 50884, 50885, 50886, 50887) c6( ) c7(an); #c1(RAB29) c2(XP_005245628) c3(ras-related protein Rab-7L1 isoform X1) c4(266806) c5(266807, 266808, 266809, 266810, 266811, 266812, 266813, 266814, 266815, 266816, 266817, 266818, 266819, 266820, 266821) c6( ) c7(an); #c1(RAB29) c2(XP_011508398) c3(ras-related protein Rab-7L1 isoform X2) c4( ) c5(299105, 299106, 299107, 299108, 299109) c6( ) c7(an); #c1(RAB2A) c2(NP_001229573) c3(ras-related protein Rab-2A isoform b) c4(84663, 84664) c5(84665, 84666, 84667, 84668, 84669, 84670, 84671, 84672, 84673, 84674, 84675) c6( ) c7(T, kl); #c1(RAB2A) c2(NP_002856) c3(ras-related protein Rab-ZA isoform a) c4(132910) c5(132911, 132912, 132913, 132914, 132915, 132916, 132917) c6( ) c7(T, kl); #c1(RAB31) c2(NP_006859) c3(ras-related protein Rab-31) c4(168672, 168673) c5(168674, 168675, 168676, 168677, 168678, 168679, 168680, 168681, 168682, 168683, 168684, 168685) c6( ) c7(aW, t, T, m, aT); #c1(RAB32) c2(NP_006825) c3(ras-related protein Rab-32) c4(168453) c5(168454, 168455, 168456, 168457, 168458, 168459) c6( ) c7(AW, fJ, lF, aA, bB, h, aF, ey, g, c1); #c1(RAB33B) c2(NP_112586) c3(ras-related protein Rab-33B) c4( ) c5(221478, 221479, 221480, 221481, 221482, 221483) c6( ) c7(cxY, aRD, Qe); #c1(RAB33B) c2(XP_011530601) c3(ras-related protein Rab-33B isoform X1) c4(326502) c5( ) c6( ) c7(cxY, aRD, de); #c1(RAB34) c2(NP_001136096) c3(ras-related protein Rab-34 isoform 2) c4(54333) c5(54334, 54335, 54336, 54337, 54338, 54339, 54340) c6( ) c7(aW, aT); #c1(RAB34) c2(NP_001136097) c3(ras-related protein Rab-34 isoform 3) c4(54341, 54342) c5(54343, 54344, 54345, 54346, 54347, 54348, 54349, 54350, 54351, 54352, 54353, 54354) c6( ) c7(aW, aT); #c1(RAB34) c2(NP_001138414) c3(ras-related protein Rab-34 isoform 4) c4(56605) c5(56606, 56607, 56608, 56609, 56610) c6( ) c7(aW, aT); #c1(RAB34) c2(NP_001138415) c3(ras-related protein Rab-34 isoform 5) c4(56611) c5(56612, 56613, 56614, 56615, 56616, 56617, 56618, 56619, 56620, 56621, 56622) c6( ) c7(aW, aT); #c1(RAB34) c2(NP_001243205) c3(ras-related protein Rab-34 isoform 6) c4(90168) c5(90169, 90170, 90171, 90172, 90173) c6( ) c7(aW, aT); #c1(RAB34) c2(NP_114140) c3(ras-related protein Rab-34 isoform 1) c4(222298, 222299) c5(222300, 222301, 222302, 222303, 222304, 222305, 222306, 222307, 222308, 222309, 222310, 222311, 222312, 222313, 222314, 222315) c6( ) c7(aW, aT); #c1(RAB35) c2(NP_001161078) c3(ras-related protein Rab-35 isoform 2) c4(65277) c5(65278, 65279, 65280, 65281, 65282, 65283, 65284, 65285, 65286, 65287, 65288, 65289, 65290, 65291) c6(65292, 65293, 65294) c7(t, T, m); #c1(RAB35) c2(NP_006852) c3(ras-related protein Rab-35 isoform 1) c4(168655, 168656) c5(168657, 168658, 168659, 168660, 168661, 168662, 168663) c6( ) c7(t, T, m); #c1(RAB35) c2(XP_011536081) c3(ras-related protein Rab-35 isoform X1) c4(333405) c5(333406, 333407, 333408, 333409, 333410, 333411, 333412, 333413, 333414, 333415, 333416) c6(333417, 333418) c7(t, T, m); #c1(RAB36) c2(NP_004905) c3(ras-related protein Rab-36) c4(150663, 150664, 150665) c5(150666, 150667, 150668, 150669, 150670, 150671, 150672, 150673, 150674, 150675, 150676, 150677, 150678, 150679, 150680, 150681, 150682, 150683) c6( ) c7(Or); #c1(RAB36) c2(XP_011528854) c3(ras-related protein Rab-36 isoform X12) c4( ) c5(324227) c6( ) c7(Or); #c1(RAB37) c2(NP_001006639) c3(ras-related protein Rab-37 isoform 2) c4(22276, 22277) c5(22278, 22279, 22280, 22281, 22282, 22283, 22284, 22285, 22286, 22287, 22288, 22289, 22290, 22291, 22292, 22293) c6( ) c7(eM, fr, bX); #c1(RAB37) c2(NP_001157461) c3(ras-related protein Rab-37 isoform 4) c4(62189, 62190) c5(62191, 62192, 62193, 62194, 62195, 62196, 62197, 62198, 62199, 62200, 62201, 62202) c6( ) c7(eM, fr, bX); #c1(RAB37) c2(NP_001157462) c3(ras-related protein Rab-37 isoform 5) c4(62203) c5(62204, 62205, 62206, 62207, 62208, 62209) c6( ) c7(eM, fr, bX); #c1(RAB37) c2(NP_783865) c3(ras-related protein Rab-37 isoform 3) c4(251871, 251872, 251873) c5(251874, 251875, 251876, 251877, 251878, 251879, 251880, 251881, 251882, 251883, 251884, 251885, 251886, 251887, 251888, 251889, 251890, 251891, 251892, 251893, 251894, 251895) c6( ) c7(eM, fr, bX); #c1(RAB37) c2(XP_005257345) c3(ras-related protein Rab-37 isoform X1) c4(275061) c5(275062, 275063, 275064, 275065, 275066, 275067) c6( ) c7(eM, fr, bX); #c1(RAB38) c2(NP_071732) c3(ras-related protein Rab-38) c4(211851) c5(211852, 211853, 211854, 211855, 211856, 211857) c6( ) c7(AT, T, Y, lF, wt, sj); #c1(RAB39A) c2(NP_059986) c3(ras-related protein Rab-39A) c4(193503) c5(193504, 193505, 193506, 193507, 193508, 193509) c6( ) c7(bM); #c1(RAB39B) c2(NP_741995) c3(ras-related protein Rab-39B) c4(247552) c5(247553, 247554, 247555, 247556, 247557, 247558) c6( ) c7(ha, Az, nf, nA, cxZ, bN); #c1(RAB3A) c2(XP_011526466) c3(ras-related protein Rab-3A isoform X1) c4(320768) c5(320769, 320770, 320771, 320772, 320773, 320774) c6( ) c7(aW, Gx, aT, nC, bN, L); #c1(RAB3D) c2(NP_004274) c3(ras-related protein Rab-3D) c4(144764) c5(144765, 144766, 144767, 144768, 144769, 144770) c6( ) c7(aW, Fc, aT); #c1(RAB3GAP1) c2(NP_001165906) c3(rab3 GTPase-activating protein catalytic subunit isoform 1) c4(67981) c5(67982, 67983, 67984, 67985, 67986, 67987) c6( ) c7(T, if, jh, aA, ci, qN, q, aR, ad, ft, lg, aT, ab, ae, af, aJE, cxV, d, aW, ff, cxT, r, aF, en, bP, eD, m, B, L); #c1(RAB3GAP1) c2(NP_036365) c3(rab3 GTPase-activating protein catalytic subunit isoform 2) c4(173260) c5(173261, 173262, 173263, 173264, 173265, 173266) c6( ) c7(T, if, jh, aA, ci, qN, q, aR, ad, ft, lg, aT, ab, ae, af, aJE, cxV, d, aW, ff, cxT, r, aF, en, bP, eD, m, B, L); #c1(RAB3GAP1) c2(XP_011509127) c3(rab3 GTPase-activating protein catalytic subunit isoform X4) c4(299744) c5(299745, 299746, 299747, 299748, 299749, 299750) c6( ) c7(T, if, jh, aA, ci, qN, q, aR, ad, ft, lg, aT, ab, ae, af, aJE, cxV, d, aW, ff, cxT, r, aF, en, bP, eD, m, B, L); #c1(RAB3GAP2) c2(NP_036546) c3(rab3 GTPase-activating protein non-catalytic subunit) c4(174328) c5(174329, 174330, 174331, 174332, 174333, 174334) c6( ) c7(cxV, cya, cxT, pl, m, fq, ps); #c1(RAB3IL1) c2(NP_001258615) c3(guanine nucleotide exchange factor for Rab-3A isoform 2) c4(96192) c5(96193, 96194, 96195, 96196, 96197, 96198) c6( ) c7(bzm, bH); #c1(RAB3IL1) c2(NP_037533) c3(guanine nucleotide exchange factor for Rab-3A isoform 1) c4(176068) c5(176069, 176070, 176071, 176072, 176073, 176074) c6( ) c7(bzm, bH); #c1(RAB3IP) c2(NP_001019818) c3(rab-3A-interacting protein isoform A) c4( ) c5(27919, 27920, 27921, 27922, 27923, 27924, 27925, 27926, 27927, 27928, 27929) c6( ) c7(q, r); #c1(RAB3IP) c2(NP_783322) c3(rab-3A-interacting protein isoform alpha 2) c4(251630, 251631, 251632, 251633) c5(251634, 251635, 251636, 251637, 251638, 251639, 251640, 251641) c6( ) c7(q, r); #c1(RAB3IP) c2(NP_783324) c3(rab-3A-interacting protein isoform beta 2) c4(251642, 251643) c5(251644, 251645, 251646, 251647, 251648, 251649, 251650, 251651, 251652) c6( ) c7(q, r); #c1(RAB3IP) c2(XP_006719289) c3(rab-3A-interacting protein isoform X1) c4(292388) c5(292389, 292390, 292391, 292392, 292393, 292394, 292395) c6( ) c7(q, r); #c1(RAB3IP) c2(XP_006719290) c3(rab-3A-interacting protein isoform X2) c4( ) c5(292396, 292397, 292398, 292399, 292400, 292401, 292402, 292403, 292404, 292405, 292406, 292407, 292408, 292409, 292410) c6( ) c7(q, r); #c1(RAB4DAL) c2(NP_001027004) c3(ras-related protein Rab-40A-like) c4(30055) c5(30056, 30057, 30058, 30059, 30060, 30061) c6( ) c7(aW, Az, aT, nf, uY, fd, cyb, xb); #c1(RAB40B) c2(NP_006813) c3 (ras-related protein Rab-40B) c4(168307, 168308) c5(168309, 168310, 168311, 168312, 168313, 168314, 168315, 168316, 168317, 168318, 168319, 168320, 168321, 168322, 168323, 168324) c6( ) c7(T, if, ht, jh, aA, bB, ci, qN, hF, x, av, aT, bK, K, j, hu, pi, aW, Y, ff, aF, en, g, cm, bz, bN, ck, es, qJ, iA, bE, dc, hk, uJ, np, k, bt, kS, B); #c1(RAB40C) c2(NP_001166136) c3(ras-related protein Rab-40C isoform a) c4(68286) c5(68287, 68288, 68289, 68290, 68291, 68292, 68293, 68294, 68295, 68296, 68297, 68298, 68299, 68300, 68301, 68302, 68303, 68304) c6( ) c7(aW, fd, aA, uY, aT, aF); #c1(RAB4A) c2(NP_004569) c3(ras-related protein Rab-4A isoform 1) c4(147815) c5(147816, 147817, 147818, 147819, 147820, 147821) c6( ) c7(iM, je, Y, fN, ez, h, cy, eZ, dy, fl, eo, pm, f, V, mC); #c1(RAB4B) c2(NP_057238) c3(ras-related protein Rab-4B) c4(189641, 189642) c5(189643, 189644, 189645, 189646, 189647, 189648, 189649, 189650, 189651, 189652, 189653, 189654) c6( ) c7(at, ad); #c1(RAB5A) c2(NP_001278977) c3(ras-related protein Rab-5A isoform 2) c4(111165) c5(111166, 111167, 111168, 111169, 111170, (11171, 111172, 111173, 111174, 111175, 111176) c6( ) c7(bM, T, OI, if, jx, j, fr, qN, iu, sO, ds, aT, h, SH, By, afu, B, Du, lo, f, t, bz, c, eM, m, AN); #c1(RAB5A) c2(NP_004153) c3(ras-related protein Rab-5A isoform 1) c4(143925, 143926) c5(143927, 143928, 143929, 143930, 143931, 143932) c6( ) c7(bM, T, Ol, if, jx, j, fr, qN, iu, sO, ds, aT, h, SH, By, afu, B, Du, lo, f, t, bz, c, eM, m, AN); #c1(RAB5B) c2(NP_001238966) c3(ras-related protein Rab-5B isoform 2) c4(88079) c5(88080, 88081, 88082, 88083, 88084, 88085, 88086, 88087, 88088, 88089, 88090, 88091) c6( ) c7(Y, A, qj); #c1(RAB5B) c2(XP_005269109) c3(ras-related protein Rab-5B isoform X1) c4(282306, 282307, 282308) c5(282309, 282310, 282311, 282312, 282313, 282314, 282315, 282316, 282317, 282318, 282319, 282320, 282321, 282322, 282323, 282324) c6( ) c7(Y, A, qj); #c1(RAB5C) c2(NP_001238968) c3(ras-related protein Rab-5C isoform b) c4(88092, 88093, 88094, 88095) c5(88096, 88097, 88098, 88099, 88100, 88101, 88102, 88103, 88104, 88105, 88106) c6( ) c7(T); #c1(RAB5C) c2(XP_011523393) c3(ras-related protein Rab-5C isoform X1) c4(317914) c5(317915, 317916, 317917, 317918, 317919, 317920, 317921, 317922, 317923, 317924, 317925, 317926, 317927, 317928, 317929, 317930, 317931, 317932, 317933, 317934, 317935, 317936, 317937, 317938, 317939, 317940) c6( ) c7(T); #c1(RAB6A) c2(NP_001230647) c3(ras-related protein Rab-6A isoform d) c4(86408) c5(86409, 86410, 86411, 86412, 86413, 86414, 86415, 86416, 86417, 86418, 86419, 86420) c6( ) c7(AT, T, cc, oV, ae, af, ad); #c1(RAB8A) c2(NP_001230648) c3(ras-related protein Rab-6A isoform c) c4( ) c5(86421, 86422, 86423, 86424, 86425) c6( ) c7(AT, T, cc, oV, ae, af, ad); #c1(RAB6A) c2(NP_002860) c3(ras-related protein Rab-6A isoform a) c4(132918, 132919, 132920, 132921, 132922) c5(132923, 132924, 132925, 132926, 132927, 132928, 132929, 132930, 132931, 132932, 132933, 132934, 132935, 132936, 132937, 132938, 132939, 132940, 132941) c6( ) c7(AT, T, cc, oV, ae, af, ad); #c1(RAB6A) c2(NP_942599) c3(ras-related protein Rab-6A isoform b) c4(260589, 260590, 260591) c5(260592, 260593, 260594, 260595, 260596, 260597, 260598, 260599, 260600, 260601, 260602, 260603, 260604, 260605, 260606, 260607) c6( ) c7(AT, T, cc, oV, ae, af, ad); #c1(RAB6B) c2(NP_057661) c3(ras-related protein Rab-6B) c4(192166) c5(192167, 192168, 192169, 192170, 192171, 192172, 192173, 192174, 192175, 192176, 192177) c6( ) c7(T, vT, aW, aT, K, cm, dR); #c1(RAB6B) c2(XP_011511195) c3(ras-related protein Rab-6B isoform X1) c4(301659) c5(301660, 301661, 301662, 301663, 301664, 301665) c6( ) c7(T, vT, aW, aT, K, cm, dR); #c1(RAB6C) c2(NP_115520) c3(ras-related protein Rab-6C) c4(223064) c5(223065, 223066, 223067, 223068, 223069, 223070, 223071, 223072, 223073, 223074, 223075) c6( ) c7(aW, ac, aT); #c1(RAB7A) c2(NP_004628) c3(ras-related protein Rab-7a) c4(148282, 148283) c5(148284, 148285, 148286, 148287, 148288, 148289, 148290, 148291, 148292, 148293, 148294, 148295) c6( ) c7(aJO, AT, T, arY, xU, Pt, aFa, ds, sO, Y, XC, bhc, h, j, fu, bX, bU, bV, cm, AN, afu); #c1(RAB8A) c2(NP_005361) c3(ras-related protein Rab-8A) c4(155433) c5(155434, 155435, 155436, 155437, 155438, 155439) c6( ) c7(ck, aJO, T, FR, bpj, s, aW, Y, al, aDO, eA, j, y, nC, KS, B, iP, de); #c1(RAB8B) c2(NP_057614) c3(ras-related protein Rab-8B) c4(191768, 191769) c5(191770, 191771, 191772, 191773, 191774, 191775, 191776, 191777, 191778, 191779, 191780, 191781) c6( ) c7(ey, alA); #c1(RAB9A) c2(NP_001182257) c3(ras-related protein Rab-9A) c4(74756) c5(74757, 74758, 74759, 74760, 74761, 74762) c6( ) c7(cH, XC); #c1(RABAC1) c2(NP_006414) c3(prenylated Rab acceptor protein 1) c4(164816) c5(164817, 164818, 164819, 164820, 164821, 164822) c6( ) c7(h, wR, T, eA); #c1(RABEP1) c2(NP_001077054) c3(rab GTPase-binding effector protein 1 isoform 2) c4(37605) c5(37606, 37607, 37608, 37609, 37610, 37611, 37612, 37613, 37614, 37615, 37616, 37617, 37618, 37619, 37620, 37621) c6( ) c7(dz, aF, aA, aT); #c1(RABEP1) c2(NP_001278510) c3(rab GTPase-binding effector protein 1 isoform 3) c4(109781) c5(109782, 109783, 109784, 109785, 109786, 109787) c6( ) c7(dz, aF, aA, aT); #c1(RABEP1) c2(NP_004694) c3(rab GTPase-binding effector protein 1 isoform 1) c4(148858, 148859) c5(148860, 148861, 148862, 148863, 148864, 148865, 148866, 148867, 148868, 148869) c6( ) c7(dz, aF, aA, aT); #c1(RABEP2) c2(NP_079092) c3(rab GTPase-binding effector protein 2) c4(216969, 216970, 216971) c5(216972, 216973, 216974, 216975, 216976, 216977, 216978, 216979, 216980, 216981, 216982, 216983, 216984, 216985, 216986, 216987, 216988, 216989) c6( ) c7(qo, ff, aT, Ku, en, nA); #c1(RABEPK) c2(NP_001167623) c3(rab9 effector protein with kelch motifs isoform a) c4( ) c5(69444, 69445, 69446, 69447, 69448, 69449, 69450, 69451) c6( ) c7(bM, dt, A, Jr, z, Y, eE, afS, dV, r, cU, Hn, h, cr, eo, eZ, fe, afT, y, V, q); #c1(RABEPK) c2(NP_001167624) c3(rab9 effector protein with kelch motifs isoform b) c4( ) c5(69452, 69453, 69454, 69455, 69456) c6( ) c7(bM, dt, A, Jr, z, Y, eE, afS, dV, r, cU, Hn, h, cr, eo, eZ, fe, afT, y, V, q); #c1(RABEPK) c2(XP_005251697) c3(rab9 effector protein with kelch motifs isoform X1) c4(271049, 271050) c5(271051, 271052, 271053, 271054) c6( ) c7(bM, dt, A, Jr, z, Y, eE, afS, dV, r, cU, Hn, h, cr, eo, eZ, fe, afT, y, V, q); #c1(RABEPK) c2(XP_005251698) c3(rab9 effector protein with kelch motifs isoform X2) c4(271055) c5(271056) c6( ) c7(bM, dt, A, Jr, z, Y, eE, afS, dV, r, cU, Hn, h, cr, eo, eZ, fe, afT, y, V, q); #c1(RABEPK) c2(XP_005251699) c3(rab9 effector protein with kelch motifs isoform X3) c4(271057) c5( ) c6( ) c7(bM, dt, A, Jr, z, Y, eE, afS, dV, r, cU, Hn, h, cr, eo, eL, fe, afT, y, V, q); #c1(RABEPK) c2(XP_005251700) c3(rab9 effector protein with kelch motifs isoform X4) c4( ) c5(271058, 271059, 271060, 271061, 271062, 271063, 271064, 271065) c6( ) c7(bM, dt, A, Jr, z, Y, eE, afS, dV, r, cU, Hn, h, cr, eo, eZ, fe, afT, y, V, q); #c1(RABEPK) c2(XP_011516422) c3(rab9 effector protein with kelch motifs isoform X5) c4( ) c5(309619, 309620, 309621, 309622, 309623, 309624, 309625, 309626, 309627, 309628, 309629, 309630, 309631, 309632, 309633, 309634, 309635, 309636, 309637, 309638, 309639, 309640, 309641, 309642, 309643, 309644, 309645, 309646, 309647, 309648) c6( ) c7(bM, dt, A, Jr, z, Y, eE, afS, dV, r, cU, Hn, h, cr, eo, eZ, fe, afT, y, V, q); #c1 (RABGAP1L) c2(NP_001230692) c3(rab GTPase-activating protein 1-like isoform B) c4(86576) c5(86577, 86578, 86579, 86580, 86581, 86582, 86583, 86584, 86585, 86586) c6( ) c7(ac, Uw, eZ, cz, aT, UF, bt); #c1(RABGAP1L) c2(NP_001230693) c3(rab GTPase-activating protein I-like isoform D) c4(86587) c5(86588, 86589, 86590, 86591, 86592, 86593) c6( ) c7(ac, Uw, eZ, cz, aT, UF, bt); #c1(RABGAP1L) c2(NP_001230694) c3(rab GTPase-activating protein 1-like isoform C) c4(86594) c5(86595, 86596, 86597, 86598, 86599, 86600, 86601, 86602, 86603, 86604, 86605, 86606) c6( ) c7(ac, Uw, eZ, cz, aT, UF, bt); #c1(RABGAP1L) c2(NP_055672) c3(rab GTPase-activating protein 1-like isoform A) c4( ) c5(182648, 182649, 182650, 182651, 182652, 182653, 182654, 182655, 182656, 182657, 182658, 182659, 182660, 182661, 182662, 182663, 182664, 182665, 182666, 182667, 182668, 182669, 182670) c6( ) c7(ac, Uw, eZ, cz, aT, UF, bt); #c1(RABGAP1L) c2(XP_005245738) c3(rab GTPase-activating protein 1-like isoform X2) c4(266908) c5(266909, 266910, 266911, 266912, 266913, 266914, 266915, 266916, 266917, 266918, 266919, 266920, 266921, 266922, 266923, 266924, 266925, 266926, 266927, 266928, 266929, 266930, 266931, 266932, 266933) c6( ) c7(ac, Uw, eZ, cz, aT, UF, bt); #c1(RABGAP1L) c2(XP_011508525) c3(rab GTPase-activating protein 1-like isoform X1) c4(299226, 299227, 299228, 299229, 299230, 299231, 299232, 299233) c5(299234) c6( ) c7(ac, Uw, eZ, cz, aT, UF, bt); #c1(RABGEF1) c2(NP_001273989) c3(rab5 GDP/GTP exchange factor isoform a) c4( ) c5(105971, 105972, 105973, 105974, 105975, 105976) c6( ) c7(T, byb, aA, n, wV, bS, v, ct, q, ad, aE, dV, aT, bK, K, ae, d, aW, Y, ff, r, dN, fe, aF, en, pm, Z, t, cm, bz, ik, arJ, nM, W, nb, nB, pc, eM, cj, mf, m, y, bt, B); #c1(RABGEF1) c2(NP_001273990) c3(rab5 GDP/GTP exchange factor isoform b) c4(105977, 105978, 105979, 105980, 105981) c5(105982, 105983, 105984, 105985, 105986, 105987, 105988, 105989, 105990, 105991) c6( ) c7(T, byb, aA, n, wV, bS, v, ct, q, ad, aE, dV, aT, bK, K, ae, d, aW, Y, ff, r, dN, fe, af, en, pm, Z, t, cm, bz, ik, arJ, nM, W, nb, nB, pc, eM, cj, mf, m, y, bt, B); #c1(RABGEF1) c2(NP_055319) c3(rab5 GDP/GTP exchange factor isoform c) c4(180423) c5(180424, 180425, 180426, 180427, 180428, 180429, 180430, 180431, 180432, 180433, 180434, 180435, 180436, 180437, 180438, 180439, 180440, 180441, 180442, 180443, 180444, 180445, 180446, 180447, 180448, 180449, 180450, 180451, 180452, 180453, 180454) c6( ) c7(T, byb, aA, n, wV, bS, v, ct, q, ad, aE, dV, aT, bK, K, ae, d, aW, Y, ff, r, dN, fe, aF, en, pm, Z, t, cm, bz, ik, arJ, nM, W, nb, nB, pc, eM, cj, mf, m, y, bt, B); #c1(RAB1F) c2(NP_002862) c3(guanine nucleotide exchange factor MSS4) c4(132942) c5(132943, 132944, 132945, 132946, 132947, 132948) c6( ) c7(aE, cz); #c1(RABL6) c2(NP_001167459) c3(rab-like protein 6 isoform 3) c4(68944, 68945, 68946, 68947) c5(68948, 68949, 68950, 68951, 68952, 68953, 68954, 68955, 68956, 68957, 68958, 68959) c6( ) c7(aW, nA, aT); #c1(RABL6) c2(NP_001167460) c3(rab-like protein 6 isoform 4) c4(68960, 68961) c5(68962, 68963, 68964, 68965, 68966, 68967, 68968, 68969, 68970, 68971, 68972, 68973, 68974, 68975, 68976, 68977, 68978, 68979) c6( ) c7(aW, nA, aT); #c1(RABL6) c2(NP_078994) c3(rab-like protein 6 isoform 1) c4(216473, 216474, 216475, 216476, 216477, 216478) c5(216479, 216480, 216481, 216482, 216483, 216484, 216485, 216486, 216487, 216488, 216489, 216490, 216491, 216492, 216493, 216494, 216495, 216496, 216497, 216498, 216499, 216500, 216501, 216502, 216503, 216504, 216505, 216506, 216507, 216508, 216509, 216510, 216511, 216512, 216513, 216514, 216515, 216516, 216517, 216518, 216519, 216520) c6(216521, 216522) c7(aW, nA, aT); #c1(RACI) c2(NP_008839) c3(ras-related C3 botulinum toxin substrate 1 isoform Rac1) c4(168963) c5(168964, 168965, 168966, 168967, 168968, 168969) c6( ) c7(T, aC, pf, azi, if, al, bB, ci, bv, aE, K, aCU, LC, aW, Ls, hl, as, cm, Qt, eM, iT, mx, U, cj, n, BF, ct, nA, qN, fr, cd, ep, kH, h, By, dL, wb, Um, dx, xf, fe, kp, yq, bQL, dB, PI, at, ajW, B, wL, fG, aA, cz, q, eA, lg, aT, cy, dz, ae, jf, afG, iU, Y, r, en, t, bN, ck, ac, mf, dy, aps, zu, bt, iW, jh, bS, zU, iJ, ad, fU, av, cyc, vw, hP, ji, NP, d, lR, ff, aF, iV, Z, bz, ek, gk, Ew, yE, bE, X, aS, iu, rk); #c1(RACI) c2(NP_061485) c3(ras-related C3 botulinum toxin substrate 1 isoform Rac1b) c4(200343) c5(200344, 200345, 200346, 200347, 200348, 200349) c6( ) c7(T, aC, pf, azi, if, al, bB, ci, bv, aE, K, aCU, LC, aW, Ls, hl, as, cm, Qt, eM, iT, mx, U, cj, n, BF, ct, nA, qN, fr, cd, ep, kH, h, By, dL, wb, Um, dx, xf, fe, kp, yq, bQL, dB, PI, at, ajW, B, wL, fG, aA, cz, q, eA, lg, aT, cy, dz, ae, jf, afG, iU, Y, r, en, t, bN, ck, ac, mf, dy, aps, zu, bt, iW, jh, bS, zU, iJ, ad, fU, av, cyc, vw, hP, ji, NP, d, lR, ff, aF, iV, Z, bz, ek, gk, Ew, yE, bE, X, aS, iu, rk); #c1(RAC2) c2(NP_002863) c3(ras-related C3 botulinum toxin substrate 2) c4(132949, 132950, 132951, 132952) c5(132953, 132954, 132955, 132956, 132957, 132958, 132959, 132960, 132961, 132962, 132963, 132964, 132965, 132966, 132967, 132968, 132969, 132970, 132971, 132972, 132973, 132974, 132975, 132976) c6( ) c7(T, aC, iW, RX, jh, oY, afK, AE, ee, eA, ad, ip, cyd, ke, JX, aT, hD, h, By, K, an, ae, iU, Cw, oV, Y, CM, fe, t, ck, yE, dB, gZ, m, ajW, ajJ, bt, ra, B, rk); #c1(RAC2) c2(XP_006724349) c3(ras-related C3 botulinum toxin substrate 2 isoform X1) c4(295947) c5(295948, 295949, 295950, 295951, 295952, 295953) c6( ) c7(T, aC, iW, RX, jh, oY, afK, AE, ee, eA, ad, ip, cyd, ke, JX, aT, hD, h, By, K, an, ae, iU, Cw, oV, Y, CM, fe, t, ck, yE, dB, gZ, m, ajW, ajJ, bt, ra, B, rk); #c1(RAC3) c2(NP_005043) c3(ras-related C3 botulinum toxin substrate 3) c4(152408) c5(152409, 152410, 152411, 152412, 152413, 152414) c6( ) c7(T, iU, dB, aW, aT, n, ci, ae, eA, ad); #c1(RACGAP1) c2(XP_011536537) c3(rac GTPase-activating protein 1 isoform X2) c4(333914, 333915, 333916, 333917, 333918) c5(333919, 333920, 333921, 333922, 333923, 333924, 333925, 333926, 333927, 333928, 333929, 333930, 333931) c6( ) c7(T, hl, cz, n, nA, ad, ip, hX, iu, aE, aT, cye, dL, hP, K, ey, ae, eL, cxV, kl, iU, aW, r, eE, bz, Ew, eM, B, afH); #c1(RACGAP1) c2(XP_011536542) c3(rac GTPase-activating protein 1 isoform X1) c4(333932, 333933, 333934, 333935, 333936) c5(333937, 333938, 333939, 333940, 333941, 333942, 333943, 333944, 333945, 333946, 333947, 333948, 333949, 333950, 333951, 333952, 333953, 333954, 333955, 333956, 333957, 333958, 333959, 333960, 333961, 333962, 333963, 333964, 333965, 333966, 333967, 333968, 333969, 333970, 333971, 333972, 333973, 333974, 333975, 333976, 333977, 333978, 333979, 333980, 333981, 333982, 333983, 333984, 333985, 333986, 333987, 333988, 333989, 333990, 333991, 333992, 333993, 333994, 333995, 333996, 333997, 333998, 333999, 334000, 334001, 334002, 334003) c6( ) c7(T, hl, cz, n, nA, ad, ip, hX, iu, aE, aT, cye, dL, hP, K, ey, ae, eL, cxV, kl, iU, aW, r, eE, bz, Ew, eM, B, afH); #c1(RAD1) c2(NP_002844) c3(cell cycle checkpoint protein RAD1) c4(132729, 132730) c5(132731, 132732, 132733, 132734, 132735, 132736, 132737, 132738, 132739, 132740, 132741, 132742, 132743, 132744, 132745, 132746) c6( ) c7(bL, cF, ds, jh, cj, aT, aj, J); #c1(RAD17) c2(NP_579917) c3(cell cycle checkpoint protein RAD17 isoform 2) c4(234720) c5(234721, 234722, 234723, 234724, 234725, 234726, 234727, 234728, 234729, 234730, 234731, 234732) c6( ) c7(av, T, eG, aE, eM, bE, aW, wu, aT, hy, ci, K, wm, ik, bz); #c1(RAD17) c2(NP_579919) c3(cell cycle checkpoint protein RAD17 isoform 4) c4(234733) c5(234734, 234735, 234736, 234737, 234738, 234739) c6( ) c7(av, T, eG, aE, eM, bE, aW, wu, aT, hy, ci, K, wm, ik, bz); #c1(RAD17) c2(NP_579922) c3(cell cycle checkpoint protein RAD(7 isoform 1) c4(234740, 234741, 234742) c5(234743, 234744, 234745, 234746, 234747, 234748, 234749, 234750, 234751, 234752, 234753, 234754, 234755, 234756, 234757, 234758, 234759, 234760, 234761, 234762, 234763, 234764, 234765, 234766, 234767, 234768, 234769, 234770, 234771, 234772, 234773, 234774, 234775, 234776, 234777, 234778, 234779, 234780, 234781, 234782, 234783, 234784) c6( ) c7(av, T, eG, aE, eM, bE, aW, wu, aT, hy, ci, K, wm, iK, bz); #c1(RAD17) c2(XP_011545257) c3(cell cycle checkpoint protein RAD17 isoform X1) c4(344059, 344060) c5(344061, 344062, 344063, 344064, 344065, 344066, 344067, 344068, 344069, 344070, 344071, 344072, 344073, 344074) c6( ) c7(av, T, eG, aE, eM, bE, aW, wu, aT, hy, ci, K, wm, ik, bz); #c1(RAD18) c2(NP_064550) c3(E3 ubiquitin-protein ligase RAD18) c4(202944, 202945) c5(202946, 202947, 202948, 202949, 202950, 202951, 202952, 202953, 202954, 202955, 202956, 202957) c6( ) c7(av, T, os, eM, r, aT, lo, oZ, ci, g, bz); #c1(RAD21) c2(NP_006256) c3(double-strand-break repair protein rad21 homolog) c4(163323) c5(163324, 163325, 163326, 163327, 163328, 163329) c6( ) c7(T, auW, bB, ci, Do, aYh, q, ad, vJ, aT, ae, aW, ff, r, en, ik, BU, iT, cyf, bE, cj, aNL); #c1(RAD21L1) c2(NP_001130038) c3(double-strand-break repair protein rad21-like protein 1) c4(52459, 52460, 52461) c5(52462, 52463, 52464, 52465, 52466, 52467, 52468, 52469, 52470, 52471, 52472, 52473, 52474, 52475, 52476, 52477) c6( ) c7(V); #c1(RAD21L1) c2(XP_011527620) c3(double-strand-break repair protein rad21-like protein 1 isoform X4) c4( ) c5(322509, 322510, 322511, 322512, 322513, 322514) c6( ) c7(V); #c1(RAD23A) c2(NP_001257292) c3(UV excision repair protein RAD23 homolog A isoform 3) c4(95074) c5(95075, 95076, 95077, 95078, 95079, 95080) c6( ) c7(Y, CT, aA, aT, h, K, aan, V); #c1(RAD23A) c2(NP_005044) c3(UV excision repair protein RAD23 homolog A isoform 1) c4(152415, 152416) c5(152417, 152418, 152419, 152420, 152421, 152422, 152423, 152424, 152425, 152426, 152427, 152428, 152429, 152430, 152431, 152432) c6( ) c7(Y, CT, aA, aT, h, K, aan, V); #c1(RAD23B) c2(NP_001231642) c3(UV excision repair protein RAD23 homolog B isoform 2) c4(87383, 87384) c5(87385, 87386, 87387, 87388, 87389, 87390) c6( ) c7(GI, T, gU, ht, fd, Gn, V, eA, ad, ip, Qk, aT, dL, K, hu, eL, aW, Az, nY, zE, bz, fq, eM, dB, cj, at, aan, rA, B); #c1(RAD23B) c2(NP_001231653) c3(UV excision repair protein RAD23 homolog B isoform 3) c4(87391) c5(87392, 87393, 87394, 87395, 87396, 87397, 87398, 87399, 87400, 87401, 87402, 87403, 87404, 87405, 87406, 87407, 87408, 87409, 87410, 87411, 87412, 87413, 87414) c6( ) c7(GI, T, gU, ht, fd, Gn, V, eA, ad, ip, Qk, aT, dL, K, hu, eL, aW, Az, nY, zE, bz, fq, eM, dB, cj, at, aan, rA, B); #c1(RAD23B) c2(NP_002865) c3(UV excision repair protein RAD23 homolog B isoform 1) c4(132977, 132978, 132979) c5(132980, 132981, 132982, 132983, 132984, 132985, 132986) c6( ) c7(GI, T, gU, ht, fd, Gn, V, eA, ad, ip, Qk, aT, dL, K, hu, eL, aW, Az, nY, zE, bz, fq, eM, dB, cj, at, aan, rA, B); #c1(RAD50) c2(NP_005723) c3(DNA repair protein RAD50) c4(158847, 158848) c5(158849, 158850, 158851, 158852, 158853, 158854, 158855, 158856, 158857, 158858, 158859, 158860) c6( ) c7(bM, T, os, Dw, aC, WL, cyg, iW, jh, fd, oZ, Gn, bRy, ci, q, ad, eA, Bj, rA, aT, Ds, dL, ae, BO, aW, ff, r, Xk, en, g, t, eE, dr, aj, ck, iT, dB, m, bC, eg, bt); #c1(RAD51) c2(NP_001157742) c3(DNA repair protein RAD51 homolog 1 isoform 3) c4(62478, 62479, 62480, 62481) c5(62482, 62483, 62484, 62485, 62486, 62487, 62488, 62489, 62490, 62491, 62492, 62493, 62494, 62495, 62496, 62497, 62498, 62499, 62500, 62501, 62502, 62503, 62504, 62505, 62506, 62507, 62508, 62509) c6( ) c7(T, os, aC, if, fd, bB, ci, ip, aE, Qk, K, hu, eG, aW, g, eF, bw, iP, eM, nB, iT, vA, cj, iS, m, ht, qN, eW, br, cc, h, dL, ey, CM, op, YE, dB, hL, at, cyh, B, aA, hy, V, q, eA, eJ, aT, qv, ae, jf, qw, wr, s, iU, Pg, r, en, cl, t, iK, ck, kP, np, fD, bt, ao, Mb, ZI, iW, jh, oZ, hF, ad, av, asi, hd, Db, oW, fb, qt, ff, eH, aF, HI, bz, bE); #c1(RAD51) c2(NP_002866) c3(DNA repair protein RAD51 homolog 1 isoform 1) c4(132987, 132988) c5(132989, 132990, 132991, 132992, 132993, 132994, 132995, 132996) c6( ) c7(T, os, aC, if, fd, bB, ci, ip, aE, Qk, K, hu, eG, aW, g, eF, bw, iP, eM, nB, iT, vA, cj, iS, m, ht, qN, eW, br, cc, h, dL, ey, cM, op, YE, dB, hL, at, cyh, B, aA, hy, V, q, eA, eJ, aT, qv, ae, jf, qw, wr, s, iU, Pg, r, en, cl, t, ik, ck, kP, np, fD, bt, ao, Mb, ZI, iW, jh, oZ, hF, ad, av, asi, hd, Db, oW, fb, qt, ff, eH, aF, HI, bz, bE); #c1(RAD51) c2(NP_597994) c3(DNA repair protein RAD51 homolog 1 isoform 2) c4(235223, 235224, 235225) c5(235226, 235227, 235228, 235229, 235230, 235231, 235232, 235233, 235234, 235235) c6( ) c7(T, os, aC, if, fd, bB, ci, ip, aE, Qk, K, hu, eG, aW, g, eF, bw, iP, eM, nB, lT, vA, cj, iS, m, ht, qN, eW, br, cc, h, dL, ey, cM, op, YE, dB, hL, at, cyh, B, aA, hy, V, q, eA, eJ, aT, qv, ae, jf, qw, wr, s, iU, Pg, r, en, cl, t, iK, ck, kP, np, fD, bt, ao, Mb, ZI, iW, jh, oZ, hF, ad, av, asi, hd, Db, oW, fb, qt, ff, eH, aF, HI, bz, bE); #c1(RAD51) c2(XP_011520164) c3(DNA repair protein RAD51 homolog 1 isoform X3) c4( ) c5(315029, 315030, 315031, 315032, 315033, 315034, 315035, 315036, 315037, 315038, 315039, 315040) c6( ) c7(T, os, aC, if, fd, bB, ci, ip, aE, Qk, K, hu, eG, aW, g, eF, bw, iP, eM, nB, iT, vA, cj, iS, m, ht, qN, eW, br, cc, h, dL, ey, cM, op, YE, dB, hL, at, cyh, B, aA, hy, V, q, eA, eJ, aT, qv, ae, jf, qw, wr, s, iU, Pg, r, en, cl, t, ik, ck, kP, np, fD, bt, ao, Mb, ZI, iW, jh, oZ, hF, ad, av, asi, hd, Db, oW, fb, qt, ff, eH, aF, HI, bz, bE); #c1(RAD51AP1) c2(NP_001124334) c3(RAD51-associated protein 1 isoform a) c4(48720) c5(48721, 48722, 48723, 48724, 48725, 48726) c6( ) c7(ck, Mb, jz, K, ajm); #c1(RAD51AP1) c2(NP_006470) c3(RAD51-associated protein 1 isoform b) c4(165438, 165439) c5(165440, 165441, 165442, 165443, 165444, 165445, 165446, 165447, 165448, 165449, 165450, 165451) c6( ) c7(ck, Mb, jz, K, ajm); #c1(RAD51AP1) c2(XP_011519213) c3(RAD51-associated protein 1 isoform X1) c4(313803) c5(313804, 313805, 313806, 313807, 313808, 313809) c6( ) c7(ck, Mb, jz, K, ajm); #c1(RAD51B) c2(NP_002868) c3(DNA repair protein RAD51 homolog 2 isoform 1) c4(133004, 133005, 133006) c5(133007, 133008, 133009, 133010, 133011, 133012) c6( ) c7(wL, pd, V, eA, aT, aed, bc, eG, zQ, d, PE, aW, aeU, t, op, kP, nb, W, dB, vA, m, y, lB); #c1(RAD51B) c2(NP_598193) c3(DNA repair protein RAD51 homolog 2 isoform 3) c4(235384) c5(235385, 235386, 235387, 235388, 235389, 235390) c6( ) c7(wL, pd, V, eA, aT, aed, bc, eG, zQ, d, PE, aW, aeU, t, op, kP, nb, W, dB, vA, m, y, lB); #c1(RAD51B) c2(NP_598194) c3(DNA repair protein RAD51 homolog 2 isoform 2) c4(235391, 235392) c5(235393, 235394, 235395, 235396, 235397, 235398, 235399, 235400, 235401, 235402, 235403, 235404) c6( ) c7(wL, pd, V, eA, aT, aed, bc, eG, zQ, d, PE, aW, aeU, t, op, kP, nb, W, dB, vA, m, y, lB); #c1(RAD51B) c2(XP_011535351) c3(DNA repair protein RAD51 homolog 2 isoform X4) c4(332380) c5(332381, 332382, 332383, 332384, 332385, 332386, 332387, 332388, 332389, 332390) c6( ) c7(wL, pd, V, eA, aT, aed, bc, eG, zQ, d, PE, aW, aeU, t, op, kP, nb, W, dB, vA, m, y, lB); #c1(RAD51B) c2(XP_011535353) c3(DNA repair protein RAD51 homolog 2 isoform X6) c4( ) c5(332391, 332392, 332393, 332394, 332395, 332396, 332397, 332398, 332399, 332400, 332401, 332402) c6( ) c7(wL, pd, V, eA, aT, aed, bc, eG, zL, d, PE, aW, aeU, t, op, kP, nb, W, dB, vA, m, y, lB); #c1(RAD51C) c2(NP_002867) c3(DNA repair protein RAD51 homolog 3 isoform 2) c4(132997) c5(132998, 132999, 133000, 133001, 133002, 133003) c6( ) c7(qw, T, os, aW, afd, cyi, ik, aT, r, aA, oZ, dL, aF, m, Ld, qv, t, V, q, cyj); #c1(RAD51C) c2(NP_478123) c3(DNA repair protein RAD51 homolog 3 isoform 1) c4(230965) c5(230966, 230967, 230968, 230969, 230970, 230971) c6( ) c7(qw, T, os, aW, afd, cyi, ik, aT, r, aA, oZ, dL, aF, m, Ld, qv, t, V, q, cyj); #c1(RAD51D) c2(NP_001136043) c3(DNA repair protein RAD51 homolog 4 isoform 6) c4(54153) c5(54154, 54155, 54156, 54157, 54158, 54159, 54160, 54161, 54162, 54163) c6( ) c7(T, os, aC, BN, aW, ew, cyk, aT, cj, r, dL, hy, m, oJ, q, oZ, t, V, g, ne); #c1(RAD51D) c2(NP_002869) c3(DNA repair protein RAD51 homolog 4 isoform 1) c4(133013, 133014, 133015, 133016, 133017, 133018) c5(133019, 133020, 133021, 133022, 133023, 133024, 133025, 133026, 133027, 133028, 133029, 133030, 133031, 133032, 133033, 133034, 133035, 133036, 133037, 133038, 133039, 133040, 133041, 133042, 133043, 133044, 133045, 133046, 133047, 133048, 133049, 133050, 133051, 133052, 133053, 133054) c6( ) c7(T, os, aC, BN, aW, ew, cyk, aT, cj, r, dL, hy, m, oJ, q, oZ, t, V, g, ne); #c1(RAD51D) c2(NP_598332) c3(DNA repair protein RAD51 homolog 4 isoform 4) c4(235412) c5(235413, 235414, 235415, 235416, 235417, 235418) c6( ) c7(T, os, aC, BN, aW, ew, cyk, aT, cj, r, dL, hy, m, oJ, q, oZ, t, V, g, ne); #c1(RAD52) c2(NP_001284348) c3(DNA repair protein RAD52 homolog isoform a) c4(111733, 111734) c5(111735, 111736, 111737, 111738, 111739, 111740, 111741, 111742, 111743, 111744, 111745) c6( ) c7(T, if, ci, qN, V, eA, ip, Db, Qk, aT, dL, K, aW, t, bw, bz, ck, nB, dB, Qu, cj, at, m, iu); #c1(RAD52) c2(NP_001284349) c3(DNA repair protein RAD52 homolog isoform b) c4(111746) c5(111747, 111748, 111749, 111750, 111751, (11752) c6( ) c7(T, if, ci, qN, V, eA, ip, Db, Qk, aT, dL, K, aW, t, bw, bz, ck, nB, dB, Qu, cj, at, m, iu); #c1(RAD52) c2(NP_001284350) c3(DNA repair protein RAD52 homolog isoform c) c4( ) c5(111753, 111754, 111755, 111756, 111757) c6( ) c7(T, if, ci, qN, V, eA, ip, Db, Qk, aT, dL, K, aW, t, bw, bz, ck, nB, dB, Qu, cj, at, m, iu); #c1(RAD54B) c2(NP_001192191) c3(DNA repair and recombination protein RAD54B isoform 2) c4(82073) c5(82074, 82075, 82076, 82077, 82078) c6( ) c7(T, kl, aC, dB, aZ, bE, iW, aT, bB, uV, g, V, eA); #c1(RAD54B) c2(NP_001192192) c3(DNA repair and recombination protein RAD54B isoform 3) c4( ) c5(82079, 82080, 82081, 82082, 82083) c6( ) c7(T, kl, aC, dB, aZ, bE, iW, aT, bB, uV, g, V, eA); #c1(RAD54B) c2(NP_036547) c3(DNA repair and recombination protein RAD54B isoform 1) c4(174335, 174336, 174337) c5(174338, 174339, 174340, 174341, 174342, 174343, 174344, 174345, 174346, 174347, 174348, 174349, 174350) c6( ) c7(T, kl, aC, dB, aZ, bE, iW, aT, bB, uV, g, V, eA); #c1(RAD9A) c2(NP_004575) c3(cell cycle checkpoint control protein RAD9A isoform 1) c4(147829) c5(147830, 147831, 147832, 147833, 147834, 147835) c6( ) c7(ae, T, BN, eM, aW, yk, afS, aT, n, K, bz, iu, ad); #c1(RAD9B) c2(NP_001273461) c3(cell cycle checkpoint control protein RAD9B isoform b) c4( ) c5(104965, 104966, 104967, 104968, 104969, 104970, 104971, 104972) c6( ) c7(wu, wm); #c1(RAD9B) c2(NP_001273464) c3(cell cycle checkpoint control protein RAD9B isoform d) c4( ) c5(104973, 104974) c6( ) c7(wu, wm); #c1(RAD9B) c2(NP_689655) c3(cell cycle checkpoint control protein RAD9B isoform 1) c4(243486) c5(243487, 243488, 243489, 243490, 243491, 243492) c6( ) c7(wu, wm); #c1(RAD9B) c2(XP_011536274) c3(cell cycle checkpoint control protein RAD9B isoform X3) c4(333619) c5( ) c6( ) c7(wu, wm); #c1(RAE1) c2(XP_005260640) c3(mRNA export factor isoform X3) c4(276755, 276756, 276757) c5(276758, 276759, 276760) c6( ) c7(ck, T, aq, Ph, rH, iR, cm); #c1(RAE1) c2(XP_011527389) c3(mRNA export factor isoform X2) c4( ) c5(322091, 322092, 322093, 322094, 322095, 322096, 322097, 322098, 322099, 322100, 322101, 322102, 322103) c6( ) c7(ck, T, aq, Ph, rH, iR, cm); #c1(RAE1) c2(XP_011527391) c3(mRNA export factor isoform X1) c4( ) c5(322104, 322105, 322106, 322107, 322108, 322109, 322110, 322111, 322112, 322113, 322114, 322115, 322116, 322117) c6( ) c7(ck, T, aq, Ph, rH, iR, cm); #c1(RAET1E) c2(NP_001230254) c3(NKG2D ligand 4 isoform 2 precursor) c4(86144) c5(86145, 86146, 86147, 86148, 86149, 86150, 86151) c6( ) c7(bE, lo, m); #c1(RAET1E) c2(NP_001230256) c3(NKG2D ligand 4 isoform 3 precursor) c4(86152) c5(86153, 86154, 86155, 86156, 86157, 86158, 86159, 86160, 86161, 86162, 86163, 86164, 86165, 86166, 86167, 86168, 86169) c6( ) c7(bE, lo, m); #c1(RAET1E) c2(NP_631904) c3(NKG2D ligand 4 isoform 1 precursor) c4(238275) c5(238276, 238277, 238278, 238279, 238280, 238281) c6( ) c7(bE, lo, m); #c1(RAET1E) c2(XP_011533780) c3(NKG2D ligand 4 isoform X4) c4(329918) c5( ) c6( ) c7(bE, lo, m); #c1(RAET1E) c2(XP_011533781) c3(NKG2D ligand 4 isoform X1) c4(329919, 329920) c5(329921, 329922, 329923, 329924, 329925, 329926) c6( ) c7(bE, lo, m); #c1(RAET1L) c2(NP_570970) c3(retinoic acid early transcript IL protein precursor) c4(234429) c5(234430, 234431, 234432, 234433, 234434, 234435, 234436, 234437, 234438, 234439) c6( ) c7(OW); #c1(RAF1) c2(NP_002871) c3(RAF proto-oncogene serine/threonine-protein kinase) c4(133055, 133056) c5(133057, 133058, 133059, 133060, 133061, 133062, 133063, 133064, 133065, 133066, 133067, (33068) c6( ) c7(bM, T, fR, yS, bO, fd, bB, ci, Ap, afr, aE, pX, ajv, cyn, K, eL, cp, eG, aW, aEl, g, as, eF, Qt, iP, eM, nB, iT, mx, vy, w, nw, m, fW, de, cyl, NK, NT, aj, kv, fr, ft, jz, cc, gd, kH, dL, oS, AQ, aq, kp, XT, Qy, bP, dB, bD, B, Dw, dA, yk, jx, aA, yO, q, J, eA, zn, aup, nv, aT, aut, ae, af, Uf, s, iU, Y, r, en, cl, t, ik, Vn, ck, nM, Dy, mG, AH, zu, bt, VB, id, ow, atS, VT, jh, bS, ad, hX, dP, av, hd, Rq, cym, nl, vc, hP, lQ, fb, pq, Yi, d, BO, ff, eH, nh, aF, iR, bz, dr, Hp, es, Ew, bE, adg, y, iu); #c1(RAF1) c2(XP_005265416) c3(RAF proto-oncogene serine/threonine-protein kinase isoform X4) c4(279534) c5(279535, 279536, 279537, 279538, 279539, 279540) c6( ) c7(bM, T, fR, yS, bO, fd, bB, ci, Qp, afr, aE, pX, ajv, cyn, K, eL, cp, eG, aW, aEl, g, as, eF, Qt, iP, eM, nB, iT, mx, vy, [w, nw, m, fW, de, cyl, NK, NT, aj, kv, fr, ft, jz, cc, gd, kH, dL, oS, AQ, aq, kp, XT, Qy, bP, dB, bD, B, Dw, dA, yk, jx, aA, yD, q, J, eA, zn, aup, nv, aT, aut, ae, af, Uf, s, iU, Y, r, en, cl, t, ik, Vn, ck, nM, Dy, mG, AH, zu, bt, VB, id, ow, atS, VT, jh, bS, ad, hX, dP, av, hd, Rq, cym, nl, vc, hP, lQ, fb, pq, Yi, d, BO, ff, eH, nh, aF, iR, bz, dr, Hp, es, Ew, bE, adg, y, iu); #c1(RAF1) c2(XP_011532277) c3(RAF proto-oncogene serine/threonine-protein kinase isoform X3) c4(328343) c5(328344, 328345, 328346, 328347, 328348, 328349) c6( ) c7(bM, T, fR, yS, bO, fd, bB, ci, Qp, afr, aE, pX, ajv, cyn, K, eL, cp, eG, aW, aEl, g, as, eF, Qt, iP, eM, nB, iT, mx, vy, Qw, nw, m, fW, de, cyl, NK, NT, aj, kv, fr, ft, jz, cc, gd, KH, dL, OS, AQ, aq, kp, XT, Qy, bP, dB, bD, B, Dw, dA, yk, jx, aA, yO, q, J, eA, zn, aup, nv, aT, aut, ae, af, Uf, s, iU, Y, r, en, cl, t, ik, Vn, ck, nM, Dy, mG, AH, zu, bt, VB, id, ow, atS, VT, jh, bS, ad, hX, dP, av, hd, Rq, cym, nl, vc, hP, lQ, fb, pq, Yi, d, BO, ff, eH, nh, af, iR, bz, dr, Hp, es, Ew, bE, adg, y, iu); #c1(RAG1) c2(XP_005253098) c3(V(D)J recombination-activating protein 1 isoform X1) c4(272153) c5(272154, 272155, 272156, 272157, 272158, 272159) c6( ) c7(Ob, bM, T, dD, aGC, dA, bRe, ea, iW, jh, fd, bB, wV, eq, cuZ, x, ke, cyp, pe, hn, dL, ji, pk, bEg, oV, Y, CM, xs, eZ, zD, CF, bdl, hp, cm, bw, bz, ck, cyo, Zg, bE, cj, at, uz, wR, dy, k, ei, im, y, zA); #c1(RAG2) c2(NP_001230714) c3(V(D)J recombination-activating protein 2) c4(86691, 86692, 86693) c5(86694, 86695, 86696, 86697, 86698, 86699, 86700, 86701, 86702, 86703, 86704, 86705, 86706, 86707, 86708, 86709, 86710, 86711) c6( ) c7(T, dA, iW, jh, fd, v, fr, ci, nA, ai, aZZ, bRT, cuZ, ad, x, eW, cyp, aE, jZ, kH, Ne, aT, h, ji, ajA, pk, ae, dx, yF, Nr, lx, NE, aW, Y, oV, xs, CM, eZ, po, bdl, iV, pL, Nq, bnc, ck, ar, eL, qe, c, eM, bE, Zg, cj, wR, k, ER, yH, rH, y, st); #c1(RAI1) c2(NP_109590) c3(retinoic acid-induced protein 1) c4(219680, 219681, 219682, 219683, 219684, 219685) c5(219686, 219687, 219688, 219689, 219690, 219691, 219692, 219693, 219694, 219695, 219696, 219697, 219698, 219699, 219700, 219701, 219702, 219703, 219704, 219705, 219706, 219707, 219708, 219709, 219710, 219711, 219712, 219713, 219714, 219715, 219716, 219717, 219718, 219719, 219720, 219721, 219722) c6( ) c7(OF, v, nA, alX, blg, aaw, bK, K, an, Yi, alL, jf, VX, cH, Y, Az, bu, cm, dr, bN, hq, cj, ei, bD, biW, xb); #c1(RAI14) c2(NP_001138992) c3(ankycorbin isoform a) c4(58496, 58497, 58498) c5(58499, 58500, 58501, 58502, 58503, 58504, 58505, 58506, 58507, 58508, 58509, 58510, 58511, 58512) c6( ) c7(t, m); #c1(RAI14) c2(NP_001138994) c3(ankycorbin isoform b) c4(58513, 58514, 58515, 58516, 58517, 58518) c5(58519, 58520, 58521, 58522, 58523, 58524, 58525, 58526, 58527, 58528, 58529, 58530, 58531, 58532, 58533, 58534, 58535, 58536, 58537, 58538, 58539, 58540, 58541, 58542, 58543, 58544, 58545, 58546, 58547, 58548, 58549, 58550, 58551, 58552, 58553, 58554, 58555, 58556, 58557, 58558, 58559, 58560, 58561, 58562, 58563, 58564, 58565, 58566, 58567, 58568, 58569, 58570) c6( ) c7(t, m); #c1(RAI14) c2(NP_001138995) c3(ankycorbin isoform c) c4(58571) c5(58572, 58573, 58574, 58575, 58576, 58577) c6( ) c7(t, m); #c1(RAI14) c2(NP_00138997) c3(ankycorbin isoform d) c4(58583) c5(58584, 58585, 58586, 58587, 58588, 58589) c6( ) c7(t, m); #c1(RAI14) c2(XP_011512318) c3(ankycorbin isoform X2) c4(302879) c5(302880, 302881, 302882, 302883, 302884, 302885) c6( ) c7(t, m); #c1(RAI2) c2(NP_001166203) c3(retinoic acid-induced protein 2 isoform 2) c4(68478) c5(68479, 68480, 68481, 68482, 68483, 68484) c6( ) c7(alV); #c1(RAI2) c2(XP_006724523) c3(retinoic acid-induced protein 2 isoform X1) c4(296011) c5(296012, 296013, 296014, 296015, 296016, 296017, 296018, 296019, 296020, 296021, 296022, 296023, 296024, 296025, 296026, 296027, 296028, 296029) c6( ) c7(alV); #c1(RALA) c2(XP_006715825) c3(ras-related protein Ral-A isoform X1) c4(289826, 289827) c5(289828, 289829, 289830, 289831, 289832, 289833, 289834, 289835, 289836, 289837, 289838, 289839, 289840, 289841) c6( ) c7(aC, eM, h, Wb, dL, kp, ci, eL, B); #c1(RALB) c2(NP_002872) c3(ras-related protein Ral-B) c4( ) c5(133069, 133070, 133071, 133072, 133073, 133074, 133075, 133076, 133077, 133078, 133079, 133080, 133081, 133082, 133083, 133084, 133085, 133086) c6( ) c7(c, dL, ci, arA, aC); #c1(RALB) c2(XP_005263781) c3(ras-related protein Ral-B isoform X3) c4(278559) c5(278560, 278561, 278562, 278563, 278564, 278565, 278566, 278567, 278568) c6( ) c7(c, dL, ci, arA, aC); #c1(RALB) c2(XP_011509875) c3(ras-related protein Ral-B isoform X2) c4(300479, 300480) c5(300481) c6( ) c7(c, dL, ci, arA, aC); #c1(RALB) c2(XP_011509876) c3(ras-related protein Ral-B isoform X4) c4(300482) c5( ) c6( ) c7(c, dL, ci, arA, aC); #c1(RALBP1) c2(NP_006779) c3(raIA-binding protein 1) c4(167979, 167980) c5(167981, 167982, 167983, 167984, 167985, 167986, 167987, 167988, 167989, 167990, 167991, 167992, 167993, 167994, 167995, 167996, 167997, 167998) c6( ) c7(T, dA, yG, ha, cz, bB, aj, j, ci, EA, fr, q, J, ad, eA, av, ft, aE, nl, aT, jy, dL, K, ae, eL, eG, d, aW, Y, r, t, cm, bz, ik, ck, es, eM, iA, bE, bo, aS, m, y, iu); #c1(RALGAPA1) c2(NP_001269972) c3(ral GTPase-activating protein subunit alpha-1 isoform 3) c4(103324) c5(103325, 103326, 103327, 103328, 103329, 103330) c6( ) c7(aYc); #c1(RALGAPA1) c2(NP_001269973) c3(ral GTPase-activating protein subunit alpha-isoform 4) c4(103331) c5(103332, 103333, 103334, 103335, 103336, 103337, 103338, 103339, 103340, 103341) c6( ) c7(aYc); #c1(RALGAPA1) c2(NP_055805) c3(ral GTPase-activating protein subunit alpha-1 isoform 1) c4(183605) c5(183606, 183607, 183608, 183609, 183610, 183611, 183612, 183613, 183614, 183615, 183616, 183617) c6( ) c7(aYc); #c1(RALGAPA1) c2(NP_919277) c3(ral GTPase-activating protein subunit alpha-1 isoform 2) c4(258441, 258442) c5(258443, 258444, 258445, 258446, 258447, 258448) c6( ) c7(aYc); #c1(RALGAPA1) c2(XP_005267555) c3(ral GTPase-activating protein subunit alpha-I isoform X12) c4( ) c5(281017, 281018, 281019, 281020, 281021, 281022) c6( ) c7(aYc); #c1(RALGAPA1) c2(XP_006720161) c3(ral GTPase-activating protein subunit alpha-1 isoform X2) c4(293308) c5( ) c6( ) c7(aYc); #c1(RALGAPA2) c2(NP_065076) c3(ral GTPase-activating protein subunit alpha-2) c4( ) c5(203669, 203670, 203671, 203672, 203673, 203674, 203675, 203676, 203677, 203678, 203679, 203680, 203681, 203682, 203683, 203684, 203685, 203686, 203687, 203688, 203689, 203690, 203691, 203692, 203693, 203694, 203695, 203696, 203697) c6( ) c7(el, V, dL, hl); #c1(RALGAPA2) c2(XP_005260825) c3(ral GTPase-activating protein subunit alpha-2 isoform X2) c4(276923, 276924) c5(276925, 276926, 276927, 276928, 276929, 276930) c6( ) c7(el, V, dL, hl); #c1(RALGAPA2) c2(XP_006723662) c3(ral GTPase-activating protein subunit alpha-2 isoform X3) c4(295282) c5( ) c6( ) c7(el, V, dL, hl); #c1(RALGAPA2) c2(XP_011527611) c3(ral GTPase-activating protein subunit alpha-2 isoform X4) c4(322493, 322494, 322495, 322496) c5(322497, 322498, 322499, 322500, 322501, 322502) c6( ) c7(eL, V, dL, hl); #c1(RALGAPA2) c2(XP_011527613) c3(ral GTPase-activating protein subunit alpha-2 isoform X6) c4( ) c5(322503, 322504, 322505, 322506, 322507, 322508) c6( ) c7(el, V, dL, hl); #c1(RALGAPB) c2(NP_001269847) c3(ral GTPase-activating protein subunit beta isoform 2) c4(102948) c5(102949, 102950, 102951, 102952, 102953, 102954) c6( ) c7(ck, x, T, ft, hl, iU, bE, aW, gp, al, aT, bB, fd, bw, fr, k, hF, bt, B, hX); #c1(RALGAPB) c2(NP_065069) c3(ral GTPase-activating protein subunit beta isoform 1) c4(203585, 203586) c5(203587, 203588, 203589, 203590, 203591, 203592, 203593, 203594, 203595, 203596, 203597, 203598, 203599, 203600, 203601, 203602, 203603, 203604, 203605, 203606, 203607, 203608) c6( ) c7(ck, x, T, ft, hl, iU, bE, aW, gp, al, aT, bB, fd, bw, fr, k, hF, bt, B, hX); #c1(RALGAPB) c2(XP_005260519) c3(ral GTPase-activating protein subunit beta isoform X1) c4(276585) c5( ) c6( ) c7(ck, x, T, ft, hl, iU, bE, aW, gp, al, aT, bB, fd, bw, fr, k, hF, bt, B, hX); #c1(RALGAPB) c2(XP_005260522) c3(ral GTPase-activating protein subunit beta isoform X4) c4(276586, 276587) c5(276588, 276589, 276590, 276591, 276592, 276593, 276594, 276595, 276596, 276597, 276598, 276599) c6( ) c7(ck, x, T, ft, hl, iU, bE, aW, gp, al, aT, bB, fd, bw, fr, k, hF, bt, B, hX); #c1(RALGDS) c2(NP_001035827) c3(ral guanine nucleotide dissociation stimulator isoform 2) c4(33158, 33159, 33160) c5(33161, 33162, 33163, 33164, 33165, 33166, 33167, 33168, 33169, 33170, 33171, 33172, 33173, 33174, 33175, 33176, 33177, 33178, 33179, 33180, 33181) c6( ) c7(T, c, hk, Y, ff, kp, en, nA); #c1(RALGDS) c2(NP_001258703) c3(ral guanine nucleotide dissociation stimulator isoform 3) c4(96375) c5(96376, 96377, 96378, 96379, 96380, 96381) c6( ) c7(T, c, hk, Y, ff, kp, en, nA); #c1(RALGDS) c2(NP_001258704) c3(ral guanine nucleotide dissociation stimulator isoform 4) c4(96382, 96383) c5(96384, 96385, 96386, 96387, 96388, 96389) c6( ) c7(T, c, hk, Y, ff, kp, en, nA); #c1(RALGDS) c2(NP_001258705) c3(ral guanine nucleotide dissociation stimulator isoform 5) c4(96390) c5(96391, 96392, 96393, 96394, 96395, 96396) c6( ) c7(T, c, hk, Y, ff, kp, en, nA); #c1(RALGDS) c2(NP_006257) c3(ral guanine nucleotide dissociation stimulator isoform 1) c4(163330) c5(163331, 163332, 163333, 163334, 163335, 163336, 163337, 163338, 163339, 163340, 163341, 163342) c6( ) c7(T, c, hk, Y, ff, kp, en, nA); #c1(RALGPS1) c2(NP_001177657) c3(ras-specific guanine nucleotide-releasing factor RalGPS1 isoform 3) c4(72837) c5(72838, 72839, 72840, 72841, 72842, 72843) c6( ) c7(fq); #c1(RALGPS1) c2(NP_001177658) c3(ras-specific guanine nucleotide-releasing factor RalGPS1 isoform 2) c4(72844) c5(72845, 72846, 72847, 72848, 72849, 72850) c6( ) c7(fq); #c1(RALGPS1) c2(NP_001177659) c3(ras-specific guanine nucleotide-releasing factor RalGPS1 isoform 4) c4(72851, 72852, 72853, 72854) c5(72855, 72856, 72857, 72858, 72859, 72860, 72861, 72862, 72863, 72864, 72865, 72866, 72867, 72868, 72869, 72870, 72871, 72872, 72873, 72874, 72875, 72876, 72877, 72878) c6( ) c7(fq); #c1(RALGPS1) c2(NP_055451) c3(ras-specific guanine nucleotide-releasing factor RalGPS1 isoform 1) c4(181244) c5(181245, 181246, 181247, 181248, 181249, 181250) c6( ) c7(fq); #c1(RALGPS1) c2(XP_011517530) c3(ras-specific guanine nucleotide-releasing factor RalGPS1 isoform X4) c4(311327) c5( ) c6( ) c7(fq); #c1(RALGPS1) c2(XP_011517536) c3(ras-specific guanine nucleotide-releasing factor RalGPS1 isoform X9) c4(311328) c5(311329, 311330, 311331, 311332, 311333, 311334) c6( ) c7(fq); #c1(RALGPS1) c2(XP_011517538) c3(ras-specific guanine nucleotide-releasing factor RalGPS1 isoform X11) c4( ) c5(311335, 311336, 311337, 311338, 311339, 311340) c6( ) c7(fq); #c1(RALY) c2(NP_031393) c3(RNA-binding protein Raly isoform 2) c4(172202, 172203, 172204) c5(172205, 172206, 172207, 172208, 172209, 172210, 172211, 172212, 172213, 172214, 172215, 172216, 172217, 172218, 172219, 172220, 172221, 172222) c6( ) c7(aW, ev, r, al, q, xa); #c1(RALY) c2(NP_057951) c3(RNA-binding protein Raly isoform 1) c4(192600, 192601) c5(192602, 192603, 192604, 192605, 192606, 192607, 192608, 192609, 192610, 192611, 192612, 192613) c6( ) c7(aW, ev, r, aT, q, xa); #c1(RALYL) c2(NP_001093861) c3(RNA-binding Raly-like protein isoform 1) c4(40420, 40421) c5(40422, 40423, 40424, 40425, 40426, 40427, 40428, 40429, 40430, 40431, 40432) c6( ) c7(cm, j, ac, aT); #c1(RALYL) c2(NP_001093862) c3(RNA-binding Raly-like protein isoform 2) c4(40433) c5(40434, 40435, 40436, 40437, 40438, 40439, 40440, 40441, 40442, 40443, 40444) c6( ) c7(cm, j, ac, aT); #c1(RALYL) c2(NP_001274173) c3(RNA-binding Raly-like protein isoform 4) c4(106102) c5(106103, 106104, 106105, 106106, 106107, 106108) c6( ) c7(cm, j, ac, aT); #c1(RALYL) c2(XP_011515765) c3(RNA-binding Raly-like protein isoform X5) c4( ) c5(308567, 308568, 308569, 308570, 308571, 308572, 308573, 308574) c6( ) c7(cm, j, ac, al); #c1(RAMP1) c2(NP_005846) c3(receptor activity-modifying protein 1 precursor) c4(159837) c5(159838, 159839, 159840, 159841, 159842, 159843, 159844, 159845, 159846, 159847, 159848) c6( ) c7(gx, cZ, T, ac, nv, ea, aw, cE, cz, bgw, uu, ci, dd, ae, ad, ek); #c1(RAMP2) c2(NP_005845) c3(receptor activity-modifying protein 2 precursor) c4(159830) c5(159831, 159832, 159833, 159834, 159835, 159836) c6( ) c7(av, A, d, crt, fG, zR, cz, fQ, fF, v, dy, ci, K, bq, j, lC, g, bz, cd, y); #c1(RAMP3) c2(NP_005847) c3(receptor activity-modifying protein 3 precursor) c4(159849, 159850) c5(159851, 159852, 159853, 159854, 159855, 159856, 159857, 159858, 159859, 159860, 159861, 159862, 159863, 159864, 159865, 159866) c6( ) c7(d, aE, gZ, j, ci, eA); #c1(RAN) c2(NP_001287725) c3(GTP-binding nuclear protein Ran isoform 2) c4( ) c5(112670, 112671, 112672, 112673, 112674, 112675, 112676, 112677) c6( ) c7(T, fd, bB, hy, ww, fr, ci, zP, ad, hX, ib, vJ, il, iX, aT, h, ae, zQ, BN, B, iU, aW, t, bz, bF, es, c, bE, m, rH, yA, ne); #c1(RAN) c2(NP_006316) c3(GTP-binding nuclear protein Ran isoform 1) c4(163936, 163937, 163938, 163939) c5(163940, 163941, 163942, 163943, 163944, 163945, 163946, 163947, 163948, 163949, 163950, 163951, 163952, 163953, 163954, 163955, 163956, 163957, 163958, 163959, 163960, 163961, 163962, 163963) c6( ) c7(T, fd, bB, hy, ww, fr, ci, zP, ad, hX, ib, vJ, il, iX, aT, h, ae, zQ, BN, B, iU, aW, t, bz, bF, es, c, bE, m, rH, yA, ne); #c1(RANBP1) c2(NP_002873) c3(ran-specific GTPase-activating protein isoform 2) c4(133087) c5(133088, 133089, 133090, 133091, 133092, 133093) c6( ) c7(bE, T, Y, h, aT); #c1(RANBP1) c2(XP_011528591) c3(ran-specific GTPase-activating protein isoform X1) c4(323813) c5(323814, 323815, 323816, 323817, 323818, 323819) c6( ) c7(bE, T, Y, h, aT); #c1(RANBP1) c2(XP_011528592) c3(ran-specific GTPase-activating protein isoform X2) c4(323820) c5(323821, 323822, 323823, 323824, 323825, 323826) c6( ) c7(bE, T, Y, h, al); #c1(RANBP10) c2(NP_065901) c3(ran-binding protein 10) c4(206136, 206137) c5(206138, 206139, 206140, 206141, 206142, 206143, 206144, 206145, 206146, 206147, 206148, 206149) c6( ) c7(aA); #c1(RANBP10) c2(XP_005256127) c3(ran-binding protein 10 isoform X1) c4(273955, 273956) c5(273957, 273958, 273959, 273960, 273961, 273962, 273963) c6( ) c7(aA); #c1(RANBP10) c2(XP_005256 (29) c3(ran-binding protein 10 isoform X2) c4(273964) c5(273965, 273966, 273967, 273968, 273969, 273970, 273971, 273972, 273973, 273974, 273975) c6( ) c7(aA); #c1(RANBP17) c2(NP_075048) c3(ran-binding protein (7) c4(213571) c5(213572, 213573, 213574, 213575, 213576, 213577) c6( ) c7(cd); #c1(RANBP17) c2(XP_011532929) c3(ran-binding protein 17 isoform X3) c4(329099) c5( ) c6( ) c7(cd); #c1(RANBP17) c2(XP_011532933) c3(ran-binding protein 17 isoform X7) c4(329100) c5(329101, 329102, 329103, 329104, 329105, 329106) c6( ) c7(cd); #c1(RANBP17) c2(XP_011532939) c3(ran-binding protein 17 isoform X13) c4( ) c5(329107, 329108, 329109, 329110, 329111, 329112) c6( ) c7(cd); #c1(RANBP2) c2(NP_006258) c3(E3 SUMO-protein ligase RanBP2) c4(163343) c5(163344, 163345, 163346, 163347, 163348, 163349) c6( ) c7(ach, T, d, hb, HP, Y, bE, Tm, fd, bB, asM, h, qj, K, cyq, ci, pC, eA, dr, rp); #c1(RANBP2) c2(XP_005264062) c3(E3 SUMO-protein ligase RanBP2 isoform X5) c4(278663) c5( ) c6( ) c7(ach, T, d, hb, HP, Y, bE, Tm, fd, bB, asM, h, qj, K, cyq, ci, pC, eA, dr, rp); #c1(RANBP3) c2(NP_003615) c3(ran-binding protein 3 isoform RANBP3-a) c4(139647) c5(139648, 139649, 139650, 139651, 139652, 139653) c6( ) c7(bE, kH, bB, lo, m, t); #c1(RANBP3) c2(NP_015559) c3(ran-binding protein 3 isoform RANBP3-b) c4(171860, 171861, 171862) c5(171863, 171864, 171865, 171866, 171867, 171868, 171869, 171870, 171871, 171872, 171873, 171874, 171875, 171876, 171877, 171878, 171879, 171880, 171881, 171882) c6( ) c7(bE, kH, bB, lo, m, t); #c1(RANBP3) c2(NP_015561) c3(ran-binding protein 3 isoform RANBP3-d) c4(171883) c5(171884, 171885, 171886, 171887, 171888, 171889) c6( ) c7(bE, kH, bB, lo, m, t); #c1(RANBP3L) c2(NP_001154901) c3(ran-binding protein 3-like isoform 1) c4(61213) c5(61214, 61215, 61216, 61217, 61218, 61219) c6( ) c7(oe, j); #c1(RANBP3L) c2(NP_659437) c3(ran-binding protein 3-like isoform 2) c4(239888, 239889) c5(239890, 239891, 239892, 239893, 239894, 239895, 239896, 239897, 239898, 239899, 239900, 239901) c6( ) c7(oe, j); #c1(RANBP6) c2(NP_036548) c3(ran-binding protein 6 isoform 1) c4(174351) c5(174352, 174353, 174354, 174355, 174356, 174357) c6( ) c7(bM); #c1(RANBP9) c2(NP_005484) c3(ran-binding protein 9) c4(156629, 156630, 156631) c5(156632, 156633, 156634, 156635, 156636, 156637, 156638, 156639, 156640) c6( ) c7(aW, af, al); #c1(RANBP9) c2(XP_006715008) c3(ran-binding protein 9 isoform X2) c4( ) c5(288386, 288387, 288388, 288389, 288390) c6( ) c7(aW, af, aT); #c1(RANBP9) c2(XP_011512507) c3(ran-binding protein 9 isoform X1) c4( ) c5(303144, 303145, 303146, 303147, 303148, 303149) c6( ) c7(aW, af, aT); #c1(RANGAP1) c2(NP_001265580) c3(ran GTPase-activating protein 1) c4(98893, 98894, 98895) c5(98896, 98897, 98898, 98899, 98900, 98901, 98902, 98903, 98904, 98905, 98906, 98907, 98908, 98909) c6( ) c7(bL, ck, fM, Kv, Y, jh, RI, pk, dr, xt, dR); #c1(RANGAP1) c2(XP_005261752) c3(ran GTPase-activating protein 1 isoform X2) c4(277597) c5(277598) c6( ) c7(bL, ck, fM, Kv, Y, jh, RI, pk, dr, xt, dR); #c1(RANGAP1) c2(XP_005261753) c3(ran GTPase-activating protein 1 isoform X3) c4( ) c5(277599, 277600, 277601, 277602, 277603, 277604) c6( ) c7(bL, ck, fM, Kv, Y, jh, RI, pk, dr, xt, dR); #c1(RANGAP1) c2(XP_011528595) c3(ran GTPase-activating protein 1 isoform X4) c4(323827, 323828) c5(323829, 323830, 323831, 323832, 323833, 323834, 323835, 323836, 323837, 323838, 323839, 323840) c6( ) c7(bL, ck, fM, Kv, Y, jh, RI, pk, dr, xt, dR); #c1(RANGAP1) c2(XP_011528599) c3(ran GTPase-activating protein 1 isoform X5) c4( ) c5(323841, 323842, 323843, 323844, 323845, 323846, 323847, 323848, 323849, 323850, 323851, 323852, 323853, 323854, 323855) c6( ) c7(bL, ck, fM, Kv, Y, jh, RI, pk, dr, xt, dR); #c1(RANGRF) c2(NP_001171272) c3(ran guanine nucleotide release factor isoform B) c4(70028) c5(70029, 70030, 70031, 70032, 70033) c6( ) c7(arD, ajM); #c1(RANGRF) c2(NP_001171273) c3(ran guanine nucleotide release factor isoform C) c4(70034) c5(70035, 70036, 70037, 70038, 70039, 70040) c6( ) c7(arD, ajM); #c1(RANGRF) c2(NP_057576) c3(ran guanine nucleotide release factor isoform A) c4(191415) c5(191416, 191417, 191418, 191419, 191420, 191421, 191422) c6( ) c7(arD, ajM); #c1(RAP1B) c2(NP_001010942) c3(ras-related protein Rap-lb isoform 1 precursor) c4(24274, 24275, 24276, 24277, 24278, 24279, 24280, 24281, 24282, 24283, 24284, 24285, 24286, 24287) c5(24288, 24289, 24290, 24291, 24292, 24293, 24294, 24295, 24296, 24297, 24298, 24299, 24300, 24301, 24302, 24303, 24304, 24305, 24306, 24307, 24308, 24309, 24310, 24311, 24312, 24313, 24314, 24315, 24316, 24317, 24318, 24319, 24320, 24321, 24322, 24323, 24324, 24325, 24326, 24327, 24328, 24329, 24330, 24331, 24332, 24333, 24334, 24335, 24336, 24337, 24338, 24339, 24340, 24341, 24342, 24343, 24344, 24345, 24346, 24347, 24348, 24349, 24350, 24351, 24352, 24353, 24354, 24355, 24356, 24357, 24358, 24359) c6( ) c7(T, aE, nB, vT, aW, Db, ff, aT, cz, bw, K, fD, en, cm, it, q, dR); #c1(RAP1B) c2(NP_001238846) c3(ras-related protein Rap-lb isoform 2) c4(87949) c5(87950, 87951, 87952, 87953, 87954, 87955, 87956, 87957, 87958, 87959, 87960, 87961, 87962, 87963, 87964, 87965, 87966, 87967, 87968, 87969) c6( ) c7(T, aE, nB, vT, aW, Db, ff, aT, cz, bw, K, fD, en, cm, it, q, dR); #c1(RAP1B) c2(NP_001238850) c3(ras-related protein Rap-lb isoform 3) c4(87970, 87971) c5(87972, 87973, 87974, 87975, 87976, 87977) c6( ) c7(T, aE, nB, vT, aW, Db, ff, aT, cz, bw, K, fD, en, cm, it, q, dR); #c1(RAP1B) c2(NP_001238851) c3(ras-related protein Rap-1b isoform 4) c4(87978) c5(87979, 87980, 87981, 87982, 87983, 87984, 87985, 87986, 87987, 87988, 87989, 87990, 87991, 87992, 87993, 87994, 87995, 87996, 87997, 87998, 87999, 88000, 88001) c6( ) c7(T, aE, nB, vT, aW, Db, ff, aT, cz, bw, K, fD, en, cm, it, q, dR); #c1(RAP1GAP2) c2(NP_001093868) c3(rap1 GTPase-activating protein 2 isoform 2) c4(40454) c5(40455, 40456, 40457, 40458, 40459, 40460) c6( ) c7(aw, ac, cz); #c1(RAPIGAP2) c2(NP_055900) c3(rap1 GTPase-activating protein 2 isoform 1) c4(184316) c5(184317, 184318, 184319, 184320, 184321, 184322, 184323, 184324, 184325, 184326, 184327, 184328, 184329, 184330, 184331, 184332) c6( ) c7(aw, ac, cz); #c1(RAPIGAP2) c2(XP_011522040) c3(rap1 GTPase-activating protein 2 isoform X1) c4(316715) c5( ) c6( ) c7(aw, ac, cz); #c1(RAP1GDS1) c2(NP_001093896) c3(rap1 GTPase-GDP dissociation stimulator 1 isoform 1) c4(40517) c5(40518, 40519, 40520, 40521, 40522, 40523) c6( ) c7(x, T, eM, aW, al, hn, ci, k, bt, ae, ad); #c1(RAP1GDS1) c2(NP_001093897) c3(rap1 GTPase-GDP dissociation stimulator 1 isoform 3) c4(40524) c5(40525, 40526, 40527, 40528, 40529, 40530) c6( ) c7(x, T, eM, aW, aT, hn, ci, k, bt, ae, ad); #c1(RAP1GDS1) c2(NP_001093898) c3(rap1 GTPase-GDP dissociation stimulator 1 isoform 4) c4(40531) c5(40532, 40533, 40534, 40535, 40536, 40537) c6( ) c7(x, T, eM, aW, aT, hn, ci, k, bt, ae, ad); #c1(RAP1GDS1) c2(NP_001093899) c3(rap1 GTPase-GDP dissociation stimulator 1 isoform 5) c4(40538) c5(40539, 40540, 40541, 40542, 40543, 40544) c6( ) c7(x, T, eM, aW, aT, hn, ci, k, bt, ae, ad); #c1(RAP1GDS1) c2(NP_001093900) c3(rap1 GTPase-GDP dissociation stimulator 1 isoform 6) c4(40545) c5(40546, 40547, 40548, 40549, 40550, 40551) c6( ) c7(x, T, eM, aW, al, hn, ci, k, bt, ae, ad); #c1(RAP1GDS1) c2(NP_066982) c3(rap1 GTPase-GDP dissociation stimulator 1 isoform 2) c4(207959) c5(207960, 207961, 207962, 207963, 207964, 207965) c6( ) c7(x, T, eM, aW, aT, hn, ci, k, bt, ae, ad); #c1(RAP2A) c2(NP_066361) c3(ras-related protein Rap-Za precursor) c4(207204) c5(207205, 207206, 207207, 207208, 207209, 207210) c6( ) c7(Vn, hd, nB, aW, oe, aw, dV, aT, y, ae, ad); #c1(RAP2B) c2(NP_002877) c3(ras-related protein Rap-2b) c4(133094) c5(133095, 133096, 133097, 133098, 133099, 133100) c6( ) c7(T, vT, aW, Y, aT, ci, K, Ea, cm, dR); #c1(RAPGEF1) c2(NP_001291204) c3(rap guanine nucleotide exchange factor 1 isoform c) c4(115573) c5(115574, 115575, 115576, 115577, 115578, 115579, 115580, 115581, 115582, 115583) c6( ) c7(bL, T, ff, gX, aA, bB, m, en, t, fq); #c1(RAPGEF1) c2(NP_005303) c3(rap guanine nucleotide exchange factor 1 isoform a) c4(154796) c5(154797, 154798, 154799, 154800, 154801, 154802, 154803, 154804, 154805, 154806) c6( ) c7(bL, T, ff, gX, aA, bB, m, en, t, fq); #c1(RAPGEF1) c2(NP_941372) c3(rap guanine nucleotide exchange factor 1 isoform b) c4(260313, 260314, 260315) c5(260316, 260317, 260318, 260319, 260320, 260321, 260322, 260323, 260324, 260325, 260326, 260327, 260328, 260329, 260330, 260331, 260332, 260333, 260334) c6( ) c7(bL, T, ff, gX, aA, bB, m, en, t, fq); #c1(RAPGEF1) c2(XP_005272248) c3(rap guanine nucleotide exchange factor 1 isoform X1l) c4(283786, 283787) c5(283788, 283789, 283790, 283791, 283792, 283793) c6( ) c7(bL, T, ff, gX, aA, bB, m, en, t, fq); #c1(RAPGEF1) c2(XP_011516873) c3(rap guanine nucleotide exchange factor 1 isoform X3) c4( ) c5(310292, 310293) c6( ) c7(bL, T, ff, gX, aA, bB, m, en, t, fq); #c1(RAPGEF1) c2(XP_011516876) c3(rap guanine nucleotide exchange factor 1 isoform X8) c4(310294, 310295) c5(310296, 310297, 310298, 310299, 310300, 310301, 310302, 310303, 310304, 310305, 310306) c6( ) c7(bL, T, ff, gX, aA, bB, m, en, t, fq); #c1(RAPGEF1) c2(XP_011516877) c3(rap guanine nucleotide exchange factor 1 isoform X9) c4(310307) c5(310308, 310309, 310310, 310311, 310312, 310313) c6( ) c7(bL, T, ff, gX, aA, bB, m, en, t, fq); #c1(RAPGEF1) c2(XP_011516879) c3(rap guanine nucleotide exchange factor 1 isoform X13) c4(310314, 310315) c5(310316, 310317, 310318, 310319, 310320, 310321, 310322, 310323, 310324, 310325, 310326, 310327) c6( ) c7(bL, T, ff, gX, aA, bB, m, en, t, fq); #c1(RAPGEF1) c2(XP_011516880) c3(rap guanine nucleotide exchange factor 1 isoform X14) c4(310328, 310329) c5(310330, 310331, 310332, 310333, 310334, 310335, 310336, 310337, 310338, 310339, 310340) c6( ) c7(bL, T, ff, gX, aA, bB, m, en, t, fq); #c1(RAPGEF1) c2(XP_011516882) c3(rap guanine nucleotide exchange factor 1 isoform X17) c4( ) c5(310341, 310342, 310343, 310344, 310345) c6( ) c7(bL, T, ff, gX, aA, bB, m, en, t, fq); #c1(RAPGEF1) c2(XP_011516883) c3(rap guanine nucleotide exchange factor 1 isoform X18) c4(310346) c5(310347, 310348, 310349, 310350, 310351, 310352) c6( ) c7(bL, T, ff, gX, aA, bB, m, en, t, fq); #c1(RAPGEF2) c2(NP_055062) c3(rap guanine nucleotide exchange factor 2) c4(178675) c5(178676, 178677, 178678, 178679, 178680, 178681) c6( ) c7(eA, Hr, bA); #c1(RAPGEF3) c2(NP_001092001) c3(rap guanine nucleotide exchange factor 3 isoform a) c4(39222, 39223) c5(39224, 39225, 39226, 39227, 39228, 39229, 39230, 39231, 39232, 39233, 39234, 39235, 39236, 39237, 39238, 39239, 39240) c6( ) c7(cyr, ac, LC, Y, XY, at, E, bS, k, ct, ci, Z, ae, ad, cp, hq); #c1(RAPGEF3) c2(NP_006096) c3(rap guanine nucleotide exchange factor 3 isoform b) c4(161845, 161846, 161847) c5(161848, 161849, 161850, 161851, 161852, 161853, 161854, 161855, 161856, 161857, 161858) c6( ) c7(cyr, ac, LC, Y, XY, at, E, bS, k, ct, ci, Z, ae, ad, cp, hq); #c1(RAPGEF3) c2(XP_011536055) c3(rap guanine nucleotide exchange factor 3 isoform X3) c4(333352) c5(333353, 333354, 333355, 333356, 333357, 333358, 333359, 333360, 333361, 333362, 333363, 333364, 333365, 333366, 333367, 333368) c6( ) c7(cyr, ac, LC, Y, XY, at, E, bS, k, ct, ci, Z, ae, ad, cp, hq); #c1(RAPGEF3) c2(XP_011536058) c3(rap guanine nucleotide exchange factor 3 isoform X6) c4(333369) c5(333370, 333371, 333372, 333373, 333374, 333375) c6( ) c7(cyr, ac, LC, Y, XY, at, E, bS, k, ct, ci, Z, ae, ad, cp, hq); #c1(RAPGEF3) c2(XP_01536061) c3(rap guanine nucleotide exchange factor 3 isoform X9) c4( ) c5(333376, 333377, 333378, 333379, 333380, 333381, 333382) c6( ) c7(cyr, ac, LC, Y, XY, at, E, bS, k, ct, ci, Z, ae, ad, cp, hq); #c1(RAPGEF4) c2(NP_001093867) c3(rap guanine nucleotide exchange factor 4 isoform b) c4(40445, 40446, 40447) c5(40448, 40449, 40450, 40451, 40452, 40453) c6( ) c7(Z, bS, XY, ct, bN); #c1(RAPGEF4) c2(NP_001269828) c3(rap guanine nucleotide exchange factor 4 isoform c) c4( ) c5(102906, 102907, 102908, 102909, 102910, 102911) c6( ) c7(Z, bS, XY, ct, bN); #c1(RAPGEF4) c2(NP_001269829) c3(rap guanine nucleotide exchange factor 4 isoform d) c4(102912, 102913) c5(102914, 102915, 102916, 102917, 102918, 102919) c6( ) c7(Z, bS, XY, ct, bN); #c1(RAPGEF4) c2(NP_008954) c3(rap guanine nucleotide exchange factor 4 isoform a) c4(169748, 169749, 169750) c5(169751, 169752, 169753, 169754, 169755, 169756, 169757, 169758, 169759, 169760, 169761, 169762, 169763, 169764, 169765, 169766, 169767, 169768, 169769, 169770, 169771, 169772, 169773, 169774, 169775, 169776, 169777, 169778, 169779, 169780, 169781, 169782, 169783) c6( ) c7(Z, bS, XY, ct, bN); #c1(RAPGEF4) c2(XP_005246303) c3(rap guanine nucleotide exchange factor 4 isoform X2) c4( ) c5(267036) c6( ) c7(Z, bS, XY, ct, bN); #c1(RAPGEF5) c2(NP_036426) c3(rap guanine nucleotide exchange factor 5) c4(173767, 173768) c5(173769, 173770, 173771, 173772, 173773, 173774, 173775, 173776, 173777, 173778, 173779, 173780, 173781, 173782, 173783) c6( ) c7(bL, AB, eR, qb, e, rf, cz, cd, dG, aj, amC, aY, J, fU, cys, amB, oT, MS, nH, rx, bK, bsR, he, H, xa, A, are, Y, eZ, gl, aSO, cm, qa, bAh, ac, fP, dy, tq); #c1(RAPGEF5) c2(XP_005249971) c3(rap guanine nucleotide exchange factor 5 isoform X2) c4(269958, 269959) c5(269960) c6( ) c7(bL, AB, eR, qb, e, rf, cz, cQ, dG, aj, amC, aY, J, fU, cys, amB, oT, MS, nH, rx, bK, bsR, he, H, xa, A, are, Y, eZ, gl, aSO, cm, qa, bAh, ac, fP, dy, tq); #c1(RAPGEF5) c2(XP_011513954) c3(rap guanine nucleotide exchange factor 5 isoform X3) c4( ) c5(306376, 306377, 306378, 306379, 306380, 306381, 306382, 306383) c6( ) c7(bL, AB, eR, qb, e, rf, cz, cl, dG, aj, amC, aY, J, fU, cys, amB, oT, MS, nH, rX, bK, bsR, he, H, xa, A, are, Y, eZ, gl, aSO, cm, qa, bAh, ac, fP, dy, tq); #c1(RAPH1) c2(NP_976241) c3(ras-associated and pleckstrin homology domains-containing protein 1 isoform 3) c4( ) c5(263058, 263059, 263060, 263061, 263062, 263063, 263064, 263065, 263066, 263067, 263068, 263069) c6( ) c7(T, dA, aw, jh, bX, eW, zn, F, zK, aT, aW, eH, lo, zL, eF, zM, zJ, bE, cj, im, y, B); #c1(RAPH1) c2(NP_998754) c3(ras-associated and pleckstrin homology domains-containing protein 1 isoform 1) c4(265818, 265819, 265820, 265821) c5(265822, 265823, 265824, 265825, 265826, 265827, 265828, 265829, 265830, 265831, 265832, 265833, 265834, 265835, 265836, 265837, 265838, 265839) c6( ) c7(T, dA, aw, jh, bX, eW, zn, F, zK, aT, aW, eH, lo, zL, eF, zM, zJ, bE, cj, im, y, B); #c1(RAPH1) c2(XP_005246836) c3(ras-associated and pleckstrin homology domains-containing protein 1 isoform X1) c4(267466, 267467, 267468) c5(267469, 267470, 267471, 267472, 267473, 267474, 267475) c6( ) c7(T, dA, aw, jh, bX, eW, zn, F, zK, aT, aW, eH, lo, zL, eF, zM, zJ, bE, cj, im, y, B); #c1(RAPH1) c2(XP_011509949) c3(ras-associated and pleckstrin homology domains-containing protein 1 isoform X2) c4(300545, 300546, 300547) c5(300548, 300549, 300550, 300551, 300552, 300553, 300554, 300555, 300556, 300557, 300558, 300559, 300560, 300561, 300562, 300563) c6( ) c7(T, dA, aw, jh, bX, eW, zn, F, zK, aT, aW, eH, lo, zL, eF, zM, zJ, bE, cj, im, y, B); #c1(RAPH1) c2(XP_011509950) c3(ras-associated and pleckstrin homology domains-containing protein 1 isoform X3) c4(300564, 300565) c5(300566, 300567, 300568, 300569, 300570, 300571, 300572, 300573, 300574, 300575, 300576, 300577, 300578, 300579, 300580, 300581) c6( ) c7(T, dA, aw, jh, bX, eW, zn, F, zK, aT, aW, eH, lo, zL, eF, zM, zJ, bE, cj, im, y, B); #c1(RAPH1) c2(XP_011509951) c3(ras-associated and pleckstrin homology domains-containing protein 1 isoform X4) c4( ) c5(300582, 300583, 300584, 300585, 300586, 300587, 300588, 300589, 300590, 300591, 300592) c6( ) c7(T, dA, aw, jh, bX, eW, zn, F, zK, aT, aW, eH, lo, zL, eF, zM, zJ, bE, cj, im, y, B); #c1(RAPSN) c2(NP_005046) c3(43 kDa receptor-associated protein of the synapse isoform 1) c4(152433) c5(152434, 152435, 152436, 152437, 152438, 152439) c6( ) c7(xA, aOf, aOi, oj, aDx, cyt); #c1(RAPSN) c2(NP_116034) c3(43 kDa receptor-associated protein of the synapse isoform 2) c4(225259) c5(225260, 225261, 225262, 225263, 225264, 225265) c6( ) c7(xA, aOf, aOi, oj, aDx, cyt); #c1(RARA) c2(NP_001019980) c3(retinoic acid receptor alpha isoform 2) c4(27986, 27987) c5(27988, 27989, 27990, 27991, 27992, 27993, 27994, 27995, 27996, 27997, 27998, 27999) c6( ) c7(T, if, al, bB, ci, x, Na, K, hu, aW, g, eF, cm, bw, qJ, iT, eM, iA, dc, uJ, k, m, ul, wu, My, ht, by, n, ct, qN, br, hn, on, pi, wC, aq, CO, mo, bP, cA, hk, Be, hL, B, fM, fZ, aA, v, er, DK, aT, dz, Nc, bK, j, ae, af, iU, Y, baP, en, cl, t, bN, ck, c, np, bt, jh, css, bS, OH, hF, ad, av, wm, BN, et, ff, eH, aF, Z, iR, bz, es, bE, aVv, dX, y, kS); #c1(RARA) c2(NP_001138774) c3(retinoic acid receptor alpha isoform 4) c4(57649) c5(57650, 57651, 57652, 57653, 57654, 57655, 57656, 57657, 57658, 57659, 57660, 57661) c6( ) c7(T, if, al, bB, ci, x, Na, K, hu, aW, g, eF, cm, bw, qJ, iT, eM, iA, dc, uJ, k, m, ul, wu, My, ht, by, n, ct, qN, br, hn, on, pi, wC, aq, CA, mo, bP, cA, hk, Be, hL, B, fM, fZ, aA, v, er, DK, aT, dz, Nc, bK, jj, ae, af, iU, Y, baP, en, cl, t, bN, ck, c, np, bt, jh, css, bS, OH, hF, ad, av, wm, BN, et, ff, eH, aF, Z, iR, bz, es, bE, aVv, dX, y, kS); #c1(RARA) c2(XP_005257609) c3(retinoic acid receptor alpha isoform X3) c4(275299, 275300) c5(275301, 275302, 275303, 275304, 275305, 275306) c6( ) c7(T, if, al, bB, ci, x, Na, K, hu, aW, g, eF, cm, bw, qJ, iT, eM, iA, dc, uJ, k, m, ul, wu, My, ht, by, n, ct, qN, br, hn, on, pi, wC, aq, CA, mo, bP, cA, hk, Be, hL, B, fM, fZ, aA, v, er, DK, aT, dz, Nc, bK, j, ae, af, iU, Y, baP, en, cl, t, bN, ck, c, np, bt, jh, css, bS, oH, hF, ad, av, wm, BN, et, ff, eH, aF, Z, iR, bz, es, bE, aVv, dX, y, kS); #c1(RARA) c2(XP_005257610) c3(retinoic acid receptor alpha isoform X1) c4(275307) c5(275308, 275309, 275310, 275311, 275312, 275313, 275314, 275315, 275316, 275317, 275318) c6( ) c7(T, if, al, bB, ci, x, Na, K, hu, aW, g, eF, cm, bw, qJ, iT, eM, iA, dc, uJ, k, m, ul, wu, My, ht, by, n, ct, qN, br, hn, on, pi, wC, aq, CO, mo, bP, cA, hk, Be, hL, B, fM, fZ, aA, v, er, DK, aT, dz, Nc, bK, j, ae, af, iU, Y, baP, en, cl, t, bN, ck, c, np, bt, jh, css, bS, OH, hF, ad, av, wm, BN, et, ff, eH, aF, Z, iR, bz, es, bE, aVv, dX, y, kS); #c1(RARB) c2(NP_000956) c3(retinoic acid receptor beta isoform 1) c4(17686) c5(17687, 17688, 17689, 17690, 17691, 17692, 17693) c6( ) c7(G, T, iH, Dd, My, ht, al, if, aA, bB, n, hy, v, it, ci, qN, fr, eA, ad, hX, dP, av, avp, ft, hd, lg, QJ, ds, pX, rC, aT, FN, dL, j, hu, oi, MO, ae, af, eL, pi, wC, B, kj, s, cvs, nO, aW, Y, ff, Rr, gl, aq, aF, CQ, en, cl, g, t, cm, bz, ik, iP, oU, ck, dS, bF, Dy, eM, nB, bE, eD, cyu, bkv, wk, Be, hL, pg, m, zL, kS, X, VB, cn); #c1(RARB) c2(NP_001277145) c3(retinoic acid receptor beta isoform 3) c4(109141, 109142) c5(109143, 109144, 109145, 109146, 109147, 109148, 109149, 109150, 109151, 109152, 109153, 109154) c6( ) c7(G, T, iH, Dd, My, ht, al, if, aA, bB, n, hy, v, it, ci, qN, fr, eA, ad, hX, dP, av, avp, ft, hd, lg, QJ, ds, pX, rC, aT, FN, dL, j, hu, oi, MO, ae, af, eL, pi, wC, B, kj, s, cvs, nO, aW, Y, ff, Rr, gl, aq, af, CO, en, cl, g, t, cm, bz, ik, iP, oU, ck, dS, bF, Dy, eM, nB, bE, eD, cyu, bkv, wk, Be, hL, pg, m, zL, KS, X, VB, cn); #c1(RARB) c2(NP_001277195) c3(retinoic acid receptor beta isoform 4) c4( ) c5(109222, 109223) c6( ) c7(G, T, iH, Dd, My, ht, al, if, aA, bB, n, hy, v, it, ci, qN, fr, eA, ad, hX, dP, av, avp, ft, hd, lg, AJ, ds, pX, rC, aT, FN, dL, j, hu, oi, MO, ae, af, eL, pi, wC, B, kj, s, cvs, nO, aW, Y, ff, Rr, gl, aq, aF, CQ, en, cl, g, t, cm, bz, ik, iP, oU, ck, dS, bF, Dy, eM, nB, bE, eD, cyu, bkv, wk, Be, hL, pg, m, zL, kS, X, VB, cn); #c1(RARB) c2(NP_001277205) c3 (retinoic acid receptor beta isoform 2) c4(109224) c5(109225, 109226, 109227, 109228, 109229, 109230, 109231, 109232) c6( ) c7(G, T, iH, Dd, My, ht, al, if, aA, bB, n, hy, v, it, ci, qN, fr, eA, ad, hX, dP, av, avp, ft, hd, lg, QJ, ds, pX, rC, aT, FN, dL, j, hu, oi, MO, ae, af, eL, pi, wC, B, kj, s, cvs, nO, aW, Y, ff, Rr, gl, aq, af, CO, en, cl, g, t, cm, bz, ik, iP, ou, ck, dS, bF, Dy, eM, nB, bE, eD, cyu, bkv, wk, Be, hl, pg, m, zL, kS, X, VB, cn); #c1 (RARG) c2(NP_001036193) c3(retinoic acid receptor gamma isoform 2) c4(34047) c5(34048, 34049, 34050, 34051, 34052, 34053, 34054, 34055) c6( ) c7(nZ, G, T, fM, aC, al, Hn, Na, jz, aT, j, af, wC, aW, Y, ff, CO, en, g, cm, ik, ck, m, bt, B); #c1(RARG) c2(NP_001230660) c3(retinoic acid receptor gamma isoform 5) c4( ) c5(86513, 86514, 86515, 86516, 86517, 86518) c6( ) c7(nZ, G, T, fM, aC, al, Hn, Na, jz, aT, j, af, wC, aW, Y, ff, CO, en, g, cm, ik, ck, m, bt, B); #c1(RARG) c2(NP_001230661) c3(retinoic acid receptor gamma isoform 3) c4(86519) c5(86520, 86521, 86522, 86523, 86524, 86525) c6( ) c7(nZ, G, T, fM, aC, al, Hn, Na, jz, aT, j, af, wC, aW, Y, ff, CQ, en, g, cm, ik, ck, m, bt, B); #c1(RARG) c2(XP_005269112) c3(retinoic acid receptor gamma isoform X3) c4(282325, 282326, 282327) c5(282328, 282329, 282330) c6( ) c7(nZ, G, T, fM, aC, al, Hn, Na, jz, aT, j, af, wC, aW, Y, ff, CO, en, g, cm, ik, ck, m, bt, B); #c1(RARG) c2(XP_005269114) c3(retinoic acid receptor gamma isoform X1) c4( ) c5(282331, 282332, 282333, 282334, 282335, 282336, 282337, 282338) c6( ) c7(nZ, G, T, fM, aC, al, Hn, Na, jz, aT, j, af, wC, aW, Y, ff, CQ, en, g, cm, iK, ck, m, bt, B); #c1(RARG) c2(XP_011536930) c3(retinoic acid receptor gamma isoform X2) c4( ) c5(334598, 334599, 334600, 334601, 334602) c6( ) c7(nZ, G, T, fM, aC, al, Hn, Na, jz, aT, j, af, wC, aW, Y, ff, CQ, en, g, cm, ik, ck, m, bt, B); #c1(RARRES1) c2(NP_002879) c3(retinoic acid receptor responder protein 1 isoform 2 precursor) c4(133115) c5(133116, 133117, 133118, 133119, 133120, 133121) c6( ) c7(ck, bF, T, jz, lU, MD, bE, aW, ff, cr, aA, bB, it, ci, en, cl, ae, B, ad); #c1(RARRES1) c2(NP_996846) c3(retinoic acid receptor responder protein 1 isoform 1 precursor) c4(264755) c5(264756, 264757, 264758, 264759, 264760) c6( ) c7(ck, bF, T, jz, lU, MD, bE, aW, ff, cr, aA, bB, it, ci, en, cl, ae, B, ad); #c1(RARRES1) c2(XP_005247743) c3(retinoic acid receptor responder protein 1 isoform X1) c4( ) c5(267956) c6( ) c7(ck, bF, T, jz, lU, MD, bE, aW, ff, cr, aA, bB, it, ci, en, cl, ae, B, ad); #c1(RARRES2) c2(NP_002880) c3(retinoic acid receptor responder protein 2 precursor) c4(133122) c5(133123, 133124, 133125, 133126, 133127, 133128, 133129, 133130, 133131, 133132, 133133, 133134, 133135, 133136) c6( ) c7(bL, dD, kl, cW, aZ, Y, e, dh, ea, cr, cz, fe, mf, j, ci, v, ei, eq, ZN, ai, y); #c1(RARRES3) c2(NP_004576) c3(retinoic acid receptor responder protein 3) c4(147836, 147837) c5(147838, 147839, 147840, 147841, 147842, 147843, 147844, 147845, 147846, 147847, 147848, 147849) c6( ) c7(Dw, T, yk, if, jx, aA, ci, EA, qN, q, er, eW, hX, F, hb, Dq, ds, aT, cU, aAn, h, hn, ey, K, lQ, afu, A, d, aW, bes, Y, ff, apn, cr, eZ, aiT, lo, kp, af, CO, en, g, iV, t, ik, awi, Bv, Jr, NL, hc, AH, Uq, aiM, cj, m, y, B); #c1(RARS) c2(NP_002878) c3(arginine-tRNA ligase, cytoplasmic) c4(133101, 133102) c5(133103, 133104, 133105, 133106, 133107, 133108, 133109, 133110, 133111, 133112, 133113, 133114) c6( ) c7(ck, T, yo, pc, Js, cyv, kz, oW, cj, kD, pg, dz, dW, CE, ey, UR, bw, bt, Jo); #c1(RARS2) c2(NP_064716) c3(probable arginine-tRNA ligase, mitochondrial precursor) c4(203542, 203543, 203544) c5(203545, 203546, 203547, 203548, 203549, 203550, 203551) c6( ) c7(yr, boQ, hb, awN, cyv, aM, ha, px, UR, bgR, bcT); #c1(RARS2) c2(XP_011534251) c3(probable arginine-tRNA ligase, mitochondrial isoform X3) c4( ) c5(330609, 330610, 330611, 330612, 330613, 330614, 330615, 330616, 330617, 330618, 330619) c6( ) c7(yr, boQ, hb, awN, cyv, aM, ha, px, UR, bgR, bcT); #c1(RASA1) c2(NP_002881) c3(ras GTPase-activating protein 1 isoform 1) c4(133137, 133138, 133139) c5(133140, 133141, 133142, 133143, 133144, 133145, 133146, 133147, 133148, 133149, 133150, 133151, 133152, 133153, 133154, 133155, 133156, 133157) c6( ) c7(cyw, T, jv, hl, Uc, WA, cz, KO, kv, fr, ci, nA, zP, Cd, Cb, q, ad, Fc, UE, aHj, iu, aE, Db, kl, afQ, aFy, aT, hD, dz, dL, hP, K, ey, zy, ae, cjs, eL, Ck, cxV, cyx, zQ, iU, Ub, et, aW, eZ, r, as, bQ, eE, cM, bz, uC, dr, es, Ew, cyy, eM, nB, bE, lq, k, bt, B, afH); #c1(RASA1) c2(NP_072179) c3(ras GTPase-activating protein 1 isoform 2) c4(212784) c5(212785, 212786, 212787, 212788, 212789, 212790) c6( ) c7(cyw, T, jv, hl, Uc, WA, cz, KO, kv, fr, ci, nA, zP, Cd, Cb, q, ad, Fc, UE, aHj, iu, aE, Db, kl, afQ, aFy, aT, HD, dz, dL, hP, K, ey, zy, ae, cjs, eL, Ck, cxV, cyx, zQ, iU, Ub, et, aW, eL, r, as, bQ, eE, cM, bz, uC, dr, es, Ew, cyy, eM, nB, bE, lq, k, bt, B, afH); #c1(RASA1) c2(XP_011541827) c3(ras GTPase-activating protein 1 isoform X1) c4(340394) c5( ) c6( ) c7(cyw, T, jv, hl, Uc, WA, cz, KO, kv, fr, ci, nA, zP, Cd, Cb, q, ad, Fc, UE, aHj, iu, aE, Db, kl, afQ, aFy, aT, HD, dz, dL, hP, K, ey, zy, ae, cjs, eL, Ck, cxV, cyx, zQ, iU, Ub, et, aW, eZ, r, as, bl, eE, cM, bz, uC, dr, es, Ew, cyy, eM, nB, bE, lq, k, bt, B, afH); #c1(RASA1) c2(XP_011541828) c3(ras GTPase-activating protein 1 isoform X2) c4( ) c5(340395, 340396, 340397, 340398, 340399, 340400) c6( ) c7(cyw, T, jv, hl, Uc, WA, cz, KO, kv, fr, ci, nA, zP, Cd, Cb, q, ad, Fc, UE, aHj, iu, aE, Db, kl, afQ, aFy, aT, hD, dz, dL, hP, K, ey, zy, ae, cjs, eL, Ck, cxV, cyx, zQ, iU, Ub, et, aW, eZ, r, as, bQ, eE, cM, bz, uC, dr, es, Ew, cyy, eM, nB, bE, lq, k, bt, B, afH); #c1(RASA2) c2(NP_001290174) c3(ras GTPase-activating protein 2 isoform 2) c4(115053, 115054) c5(115055, 115056, 115057, 115058, 115059, 115060, 115061) c6( ) c7(T, Ub, bE, r, bt, ae, q, ad, dS); #c1(RASA2) c2(NP_006497) c3(ras GTPase-activating protein 2 isoform 3) c4(165728) c5(165729, 165730, 165731, 165732, 165733, 165734) c6( ) c7(T, Ub, bE, r, bt, ae, q, ad, dS); #c1(RASA2) c2(XP_011511362) c3(ras GTPase-activating protein 2 isoform X2) c4( ) c5(301932, 301933, 301934, 301935, 301936) c6( ) c7(T, Ub, bE, r, bt, ae, q, ad, dS); #c1(RASAL1) c2(NP_001180449) c3(rasGAP-activating-like protein 1 isoform 1) c4(74103) c5(74104, 74105, 74106, 74107, 74108, 74109) c6( ) c7(qJ, fU, hd, nM, s, nB, X, r, aA, bj, aF, dy, q, g, B); #c1(RASAL1) c2(NP_001180450) c3(rasGAP-activating-like protein 1 isoform 3) c4(74110) c5(74111, 74112, 74113, 74114, 74115, 74116) c6( ) c7(qJ, fU, hd, nM, s, nB, X, r, aA, bj, aF, dy, q, g, B); #c1 (RASAL1) c2(NP_004649) c3(rasGAP-activating-like protein 1 isoform 2) c4(148509) c5(148510, 148511, 148512, 148513, 148514, 148515) c6( ) c7(qJ, fU, hd, nM, s, nB, X, r, aA, bj, aF, dy, q, g, B); #c1(RASAL2) c2(NP_004832) c3(ras GTPase-activating protein nGAP isoform 1) c4(150144) c5(150145, 150146, 150147, 150148, 150149, 150150) c6( ) c7(bL, bM, aW, aT, aA, n, v, aF, fq, X); #c1(RASAL2) c2(NP_733793) c3(ras GTPase-activating protein nGAP isoform 2) c4(247120) c5(247121, 247122, 247123, 247124, 247125, 247126) c6( ) c7(bL, bM, aW, aT, aA, n, v, aF, fq, X); #c1(RASAL2) c2(XP_005245679) c3(ras GTPase-activating protein nGAP isoform X5) c4(266882) c5(266883, 266884, 266885, 266886, 266887, 266888) c6( ) c7(bL, bM, aW, aT, aA, n, v, aF, fq, X); #c1(RASAL2) c2(XP_011508468) c3(ras GTPase-activating protein nGAP isoform X1) c4( ) c5(299188, 299189, 299190, 299191, 299192) c6( ) c7(bL, bM, aW, aT, aA, n, v, aF, fq, X); #c1(RASAL2) c2(XP_011508469) c3(ras GTPase-activating protein nGAP isoform X2) c4(299193) c5(299194, 299195, 299196, 299197, 299198, 299199) c6( ) c7(bL, bM, aW, aT, aA, n, v, aF, fq, X); #c1(RASAL2) c2(XP_011508471) c3(ras GTPase-activating protein nGAP isoform X4) c4(299200) c5( ) c6( ) c7(bL, bM, aW, aT, aA, n, v, aF, fq, X); #c1(RASD1) c2(NP_001186918) c3(dexamethasone-induced Ras-related protein 1 isoform 2) c4(78818) c5(78819, 78820, 78821, 78822, 78823) c6( ) c7(bL, Ez, aW, apl, mg, aT, fd, Ex, wR, aj, J, ad); #c1(RASD1) c2(NP_057168) c3(dexamethasone-induced Ras-related protein 1 isoform 1 proprotein) c4(189274) c5(189275, 189276, 189277, 189278, 189279, 189280, 189281) c6( ) c7(bL, Ez, aW, apl, mg, aT, fd, Ex, wR, aj, J, ad); #c1(RASD2) c2(NP_055125) c3(GTP-binding protein Rhes) c4(179105) c5(179106, 179107, 179108, 179109, 179110, 179111) c6( ) c7(eV, Fo, rR, eX); #c1(RASEF) c2(NP_689786) c3(ras and EF-hand domain-containing protein) c4(243953, 243954) c5(243955, 243956, 243957, 243958, 243959, 243960) c6( ) c7(T, Dy, iU, Y, aw, aT, bz, iP); #c1(RASEF) c2(XP_005251787) c3(ras and EF-hand domain-containing protein isoform X1) c4( ) c5(271190, 271191, 271192, 271193, 271194, 271195) c6( ) c7(T, Dy, iU, Y, aw, aT, bz, iP); #c1(RASGEF1A) c2(NP_001269791) c3(ras-GEF domain-containing family member IA isoform 1) c4(102818) c5(102819, 102820, 102821, 102822, 102823, 102824, 102825) c6( ) c7(V, ahl, ad); #c1(RASGEF1A) c2(NP_660356) c3(ras-GEF domain-containing family member IA isoform 2) c4(241068) c5(241069, 241070, 241071, 241072, 241073, 241074, 241075, 241076, 241077) c6( ) c7(V, ahl, ad); #c1(RASGEF1C) c2(NP_778232) c3(ras-GEF domain-containing family member (C) c4(251462) c5(251463, 251464, 251465, 251466, 251467, 251468, 251469, 251470, 251471, 251472) c6( ) c7(cd); #c1(RASGRF1) c2(NP_001139120) c3(ras-specific guanine nucleotide-releasing factor 1 isoform 3) c4(58725) c5(58726, 58727, 58728, 58729, 58730, 58731) c6( ) c7(bM, T, dD, yS, Ol, ea, ha, EQ, aA, jh, wV, eq, q, ad, eA, afR, aE, afU, aT, bk, K, ux, ae, cC, d, aW, Y, oV, nf, aq, fe, aF, eF, t, nC, bN, cv, bF, cnj, c, eM, iT, wB, U, Be, lF, Xu, Bd, bt, B, y); #c1(RASGRF1) c2(NP_002882) c3(ras-specific guanine nucleotide-releasing factor 1 isoform 1) c4(133158, 133159) c5(133160, 133161, 133162, 133163, 133164, 133165, 133166, 133167, 133168, 133169, 133170, 133171, 133172) c6( ) c7(bM, T, dD, yS, Ol, ea, ha, EQ, aA, jh, wV, eq, q, ad, eA, afR, aE, afU, aT, bK, K, ux, ae, cC, d, aW, Y, oV, nf, aq, fe, af, eF, t, nC, bN, cv, bF, cnj, c, eM, iT, wB, U, Be, lF, Xu, Bd, bt, B, y); #c1(RASGRF1) c2(NP_722522) c3(ras-specific guanine nucleotide-releasing factor 1 isoform 2) c4(246794) c5(246795, 246796, 246797, 246798, 246799) c6( ) c7(bM, T, dD, yS, Ol, ea, ha, EQ, aA, jh, wV, eq, q, ad, eA, afR, aE, afU, aT, bK, K, ux, ae, cC, d, aW, Y, oV, nf, aq, fe, aF, eF, t, nC, bN, cv, bF, cnj, c, eM, iT, wB, U, Be, lF, Xu, Bd, bt, B, y); #c1(RASGRF2) c2(NP_008840) c3(ras-specific guanine nucleotide-releasing factor 2) c4(168970) c5(168971, 168972, 168973, 168974, 168975, 168976) c6( ) c7(Na, av, zQ, EQ, ci, zP, hS); #c1(RASGRP1) c2(NP_001122074) c3(RAS guanyl-releasing protein 1 isoform b) c4(46385, 46386, 46387, 46388) c5(46389, 46390, 46391, 46392, 46393, 46394) c6( ) c7(bL, dP, T, A, oe, eZ, kH, bK, k, bX, arJ, bt, y); #c1(RASGRP1) c2(NP_005730) c3(RAS guanyl-releasing protein 1 isoform a) c4(158924, 158925) c5(158926, 158927, 158928, 158929, 158930, 158931, 158932, 158933, 158934, 158935, 158936, 158937) c6( ) c7(bL, dP, T, A, oe, eZ, kH, bK, k, bX, arJ, bt, y); #c1(RASGRP2) c2(NP_001092141) c3(RAS guanyl-releasing protein 2) c4(39403, 39404) c5(39405, 39406, 39407, 39408, 39409, 39410, 39411, 39412, 39413, 39414, 39415, 39416, 39417, 39418, 39419, 39420, 39421, 39422, 39423) c6( ) c7(arJ, nD); #c1(RASGRP2) c2(XP_005273764) c3(RAS guanyl-releasing protein 2 isoform X2) c4( ) c5(284827) c6( ) c7(arJ, nD); #c1(RASGRP2) c2(XP_011543021) c3(RAS guanyl-releasing protein 2 isoform X1) c4( ) c5(341727) c6( ) c7(arJ, nD); #c1(RASGRP2) c2(XP_011543025) c3(RAS guanyl-releasing protein 2 isoform X3) c4(341728) c5(341729, 341730, 341731, 341732, 341733, 341734) c6( ) c7(arJ, nD); #c1(RASGRP3) c2(NP_056191) c3(ras guanyl-releasing protein 3 isoform 2) c4(186220, 186221) c5(186222, 186223, 186224, 186225, 186226, 186227, 186228, 186229, 186230, 186231, 186232, 186233, 186234, 186235, 186236, 186237, 186238, 186239, 186240, 186241, 186242, 186243, 186244, 186245, 186246, 186247, 186248, 186249, 186250, 186251, 186252) c6( ) c7(gh, T, qt, aW, Y, De, oV, eZ, aT, cz, ae, B, ad); #c1(RASGRP3) c2(XP_011531049) c3(ras guanyl-releasing protein 3 isoform X1) c4(327010, 327011, 327012, 327013, 327014) c5(327015, 327016, 327017, 327018, 327019, 327020, 327021, 327022, 327023, 327024, 327025, 327026, 327027, 327028, 327029) c6( ) c7(gh, T, qt, aW, Y, oe, oV, eZ, aT, cz, ae, B, ad); #c1(RASGRP4) c2(NP_001139674) c3(RAS guanyl-releasing protein 4 isoform b) c4(59660) c5(59661, 59662, 59663, 59664, 59665, 59666) c6( ) c7(bM, apG, ahg, j, bt, Tc); #c1(RASGRP4) c2(NP_001139675) c3(RAS guanyl-releasing protein 4 isoform c) c4(59667) c5(59668, 59669, 59670, 59671, 59672, 59673) c6( ) c7(bM, apG, ahg, j, bt, Tc); #c1(RASGRP4) c2(NP_001139676) c3(RAS guanyl-releasing protein 4 isoform d) c4(59674) c5(59675, 59676, 59677, 59678, 59679, 59680) c6( ) c7(bM, apG, ahg, j, bt, Tc); #c1(RASGRP4) c2(NP_001139678) c3(RAS guanyl-releasing protein 4 isoform f) c4(59681) c5(59682, 59683, 59684, 59685, 59686, 59687) c6( ) c7(bM, apG, ahg, j, bt, Tc); #c1(RASGRP4) c2(NP_001139679) c3(RAS guanyl-releasing protein 4 isoform g) c4(59688) c5(59689, 59690, 59691, 59692, 59693, 59694) c6( ) c7(bM, apG, ahg, j, bt, Tc); #c1(RASGRP4) c2(NP_733749) c3(RAS guanyl-releasing protein 4 isoform a) c4(246927, 246928) c5(246929, 246930, 246931, 246932, 246933, 246934, 246935, 246936, 246937, 246938, 246939, 246940) c6( ) c7(bM, apG, ahg, j, bt, Tc); #c1(RASIP1) c2(NP_060275) c3(ras-interacting protein 1) c4(195167) c5(195168, 195169, 195170, 195171, 195172, 195173) c6( ) c7(MV, btY, e, bK, M); #c1(RASIP1) c2(XP_011525357) c3(ras-interacting protein 1 isoform X3) c4( ) c5(319818) c6( ) c7(MV, btY, e, bK, M); #c1(RASL10A) c2(NP_006468) c3(ras-like protein family member 10A precursor) c4(165424, 165425) c5(165426, 165427, 165428, 165429, 165430, 165431, 165432, 165433, 165434, 165435, 165436, 165437) c6( ) c7(T, d, eA); #c1 (RASL10B) c2(NP_201572) c3(ras-like protein family member 108 precursor) c4(228272) c5(228273, 228274, 228275, 228276, 228277, 228278) c6( ) c7(kl, aw); #c1(RASL11A) c2(NP_996563) c3(ras-like protein family member (A) c4(264333) c5(264334, 264335, 264336, 264337, 264338, 264339) c6( ) c7(dP, ad, EQ); #c1(RASL11B) c2(NP_076429) c3(ras-like protein family member (IB) c4(214149) c5(214150, 214151, 214152, 214153, 214154, 214155) c6( ) c7(ea); #c1(RASL12) c2(NP_057647) c3(ras-like protein family member (2) c4(192034, 192035) c5(192036, 192037, 192038, 192039, 192040, 192041, 192042, 192043, 192044, 192045, 192046, 192047) c6( ) c7(aLS, bJq, abW, Ft, nP); #c1(RASSF1) c2(NP_001193886) c3(ras association domain-containing protein 1 isoform B) c4( ) c5(83052, 83053, 83054, 83055, 83056) c6( ) c7(T, abY, FR, hV, if, al, WV, eb, fd, ci, ip, x, aE, pX, Qk, iv, K, aDZ, hu, MO, eL, aa, li, xQ, yo, zQ, JI, aW, Ls, Qm, g, as, eF, cm, bw, Qt, iP, qJ, eM, nB, iA, go, iS, cX, k, m, zX, aAz, Or, ht, n, fr, qN, rP, bis, BI, ft, jz, blp, eV, By, dL, Dz, KV, dx, YI, MD, AQ, hQ, ahJ, aq, fe, mo, Ld, Ye, iO, Qy, bP, asl, dB, PI, hL, anx, Pr, B, it, cn, jv, dA, WT, ex, aA, aHc, AD, q, er, eA, hW, aAF, BJ, aT, yu, aAD, Tj, ae, amw, jf, if, s, nO, Y, Lf, r, aU, eS, en, bu, cl, t, apx, iK, sS, nM, kP, Dy, au, WU, bt, VB, hS, vi, Mb, id, jh, KO, OH, iJ, ad, hX, iC, av, hd, fX, ib, py, ajk, vJ, io, vc, aNR, fb, pq, WW, d, BO, ff, aeV, aoW, aF, iV, iR, bz, YR, bF, es, gk, amS, Ew, Ny, hE, iu, jr); #c1(RASSF1) c2(NP_009113) c3(ras association domain-containing protein 1 isoform A) c4(170784) c5(170785, 170786, 170787, 170788, 170789, 170790) c6( ) c7(T, abY, fR, hV, if, al, WV, eb, fd, ci, ip, x, aE, pX, Qk, iv, K, aDZ, hu, MO, eL, aa, li, xQ, yo, zQ, JI, aW, Ls, Qm, g, as, eF, cm, bw, Qt, iP, qJ, eM, nB, iA, go, iS, cX, k, m, zX, aAz, Or, ht, n, fr, qN, rP, biS, BI, ft, jz, blp, eV, By, dL, Dz, KV, dx, YI, MD, AQ, hQ, ahJ, aq, fe, mo, Ld, Ye, iO, Qy, bP, asl, dB, PI, hL, anx, Pr, B, it, cn, jv, dA, WT, eX, aA, aHc, AD, q, er, eA, hW, aAF, BJ, aT, yu, aAD, Tj, ae, amw, jf, if, s, nO, Y, Lf, r, aU, eS, en, bu, cl, t, apx, ik, sS, nM, kP, Dy, au, WU, bt, VB, hS, vi, Mb, id, jh, KO, OH, iJ, ad, hX, iC, av, hd, fx, ib, py, ajk, vJ, io, vc, aNR, fb, pq, WW, d, BO, ff, aeV, aoW, aF, iV, iR, bz, YR, bF, es, gk, amS, Ew, Ny, hE, iu, jr); #c1(RASSF1) c2(NP_733832) c3(ras association domain-containing protein 1 isoform D) c4(247218) c5(247219, 247220, 247221, 247222, 247223, 247224, 247225) c6( ) c7(T, abY, FR, hV, if, al, WV, eb, fd, ci, ip, x, aE, pX, Qk, iv, K, aDZ, hu, MO, eL, aa, li, xQ, yo, zQ, JI, aW, Ls, Am, g, as, eF, cm, bw, At, iP, qJ, eM, nB, iA, go, iS, cX, k, m, zX, aAz, Or, ht, n, fr, qN, rP, bis, BI, ft, jz, blp, eV, By, dL, Dz, KV, dx, YI, MD, AQ, hQ, ahJ, aq, fe, mo, Ld, Ye, iO, Qy, bP, asl, dB, PI, hl, anx, Pr, B, it, cn, jv, dA, WT, ex, aA, aHc, AD, q, er, eA, hW, aAF, BJ, aT, yu, aAD, Tj, ae, amw, jf, iF, s, nO, Y, Lf, r, aU, eS, en, bu, cl, t, apx, ik, sS, nM, kP, Dy, au, WU, bt, VB, hS, vi, Mb, id, jh, KO, OH, iJ, ad, hX, iC, av, hd, fx, ib, py, ajk, vJ, io, vc, aNR, fb, pq, WW, d, BO, ff, aeV, aoW, aF, iV, iR, bz, YR, bF, es, gk, amS, Ew, Ny, hE, iu, jr); #c1(RASSF1) c2(XP_011531617) c3(ras association domain-containing protein 1 isoform X1) c4(327758, 327759) c5(327760, 327761, 327762, 327763, 327764, 327765) c6( ) c7(T, abY, fR, hV, if, al, WV, eb, fd, ci, ip, x, aE, pX, Ok, iv, K, aDZ, hu, MO, eL, aa, li, xQ, yo, zQ, JI, aW, Ls, Qm, g, as, eF, cm, bw, Qt, iP, qJ, eM, nB, iA, go, iS, cX, k, m, zX, aAz, Or, ht, n, fr, qN, rP, bis, BI, ft, jz, blp, eV, By, dL, Dz, KV, dx, YI, MD, AQ, hQ, ahJ, aq, fe, mo, Ld, Ye, iO, Qy, bP, asl, dB, PI, hl, anx, Pr, B, it, cn, jv, dA, WT, eX, aA, aHc, AD, q, er, eA, hW, aAF, BJ, aT, yu, aAD, Tj, ae, amw, jf, iF, s, nO, Y, Lf, r, aU, eS, en, bu, cl, t, apx, ik, sS, nM, kP, Dy, au, WU, bt, VB, hS, vi, Mb, id, jh, KO, OH, iJ, ad, hX, iC, av, hd, fX, ib, py, ajk, vJ, io, vc, aNR, fb, pq, WW, d, BO, ff, aeV, aoW, aF, iV, iR, bz, YR, bF, es, gk, amS, Ew, Ny, HE, iu, jr); #c1(RASSF2) c2(NP_055552) c3(ras association domain-containing protein 2) c4(181903) c5(181904, 181905, 181906, 181907, 181908, 181909, 181910, 181911, 181912, 181913) c6( ) c7(T, AS, sw, if, WT, aA, ci, qN, q, av, hd, aE, aT, ey, dx, s, Y, ff, r, aF, en, t, bz, eM, nB, gX, WU, AN, cn); #c1(RASSF3) c2(NP_835463) c3(ras association domain-containing protein 3) c4(253513) c5(253514, 253515, 253516, 253517, 253518, 253519, 253520, 253521, 253522, 253523, 253524) c6( ) c7(T, OG, eM, aW, r, aT, j, eA, g, cm, ik, bz, q); #c1(RASSF3) c2(XP_011536498) c3(ras association domain-containing protein 3 isoform X3) c4(333859) c5(333860, 333861, 333862, 333863, 333864) c6( ) c7(T, OG, eM, aW, r, aT, j, eA, g, cm, ik, bz, q); #c1(RASSF4) c2(NP_114412) c3(ras association domain-containing protein 4) c4(222635, 222636, 222637) c5(222638, 222639, 222640, 222641, 222642, 222643, 222644, 222645, 222646, 222647, 222648, 222649, 222650, 222651, 222652, 222653, 222654, 222655, 222656, 222657, 222658, 222659, 222660) c6( ) c7(af, eA, Xu); #c1(RASSF4) c2(XP_005271890) c3(ras association domain-containing protein 4 isoform X5) c4(283614, 283615, 283616) c5(283617, 283618, 283619, 283620, 283621, 283622, 283623, 283624, 283625, 283626, 283627, 283628, 283629) c6( ) c7(af, eA, Xu); #c1(RASSF4) c2(XP_006718077) c3(ras association domain-containing protein 4 isoform X4) c4(291497) c5(291498, 291499, 291500, 291501, 291502, 291503) c6( ) c7(af, eA, Xu); #c1(RASSF4) c2(XP_011538557) c3(ras association domain-containing protein 4 isoform X8) c4( ) c5(336314) c6( ) c7(af, eA, Xu); #c1(RASSF5) c2(NP_872604) c3(ras association domain-containing protein 5 isoform A) c4(256490, 256491, 256492) c5(256493, 256494, 256495, 256496, 256497, 256498, 256499, 256500, 256501, 256502, 256503, 256504, 256505, 256506, 256507, 256508, 256509, 256510) c6( ) c7(T, Tb, fr, ci, q, eA, av, hd, py, aT, K, nO, aW, eZ, r, aq, g, as, cm, ik, bz, qJ, OG, eM, nB, anx, B); #c1(RASSF5) c2(NP_872605) c3(ras association domain-containing protein 5 isoform B) c4(256511) c5(256512, 256513, 256514, 256515, 256516, 256517) c6( ) c7(T, Tb, fr, ci, q, eA, av, hd, py, aT, K, nO, aW, eZ, r, aq, g, as, cm, ik, bz, qJ, OG, eM, nB, anx, B); #c1(RASSF5) c2(NP_872606) c3(ras association domain-containing protein 5 isoform C) c4(256518) c5(256519, 256520, 256521, 256522, 256523, 256524) c6( ) c7(T, Tb, fr, ci, q, eA, av, hd, py, aT, K, nO, aW, eZ, r, aq, g, as, cm, ik, bz, qJ, OG, eM, nB, anx, B); #c1(RASSF6) c2(NP_001257320) c3(ras association domain-containing protein 6 isoform c) c4( ) c5(95132, 95133, 95134, 95135, 95136) c6( ) c7(ck, T, aA, n, aF, cm, fq, ao); #c1(RASSF6) c2(NP_001257321) c3(ras association domain-containing protein 6 isoform d) c4(95137) c5(95138, 95139, 95140, 95141, 95142, 95143) c6( ) c7(ck, T, aA, n, aF, cm, fq, ao); #c1(RASSF6) c2(NP_958834) c3(ras association domain-containing protein 6 isoform b) c4(262229) c5(262230, 262231, 262232, 262233, 262234, 262235, 262236) c6( ) c7(ck, T, aA, n, aF, cm, fq, ao); #c1(RASSF6) c2(XP_011529990) c3 (ras association domain-containing protein 6 isoform X1) c4(325805) c5(325806, 325807, 325808, 325809, 325810) c6( ) c7(ck, T, aA, n, aF, cm, fq, ao); #c1(RASSF6) c2(XP_011529991) c3(ras association domain-containing protein 6 isoform X3) c4(325811) c5(325812) c6( ) c7(ck, T, aA, n, aF, cm, fq, ao); #c1(RASSF7) c2(NP_001137465) c3(ras association domain-containing protein 7 isoform 2) c4(55871) c5(55872, 55873, 55874, 55875, 55876, 55877, 55878, 55879) c6( ) c7(T, pY, ko, q, er, av, aT, iv, dL, bK, eL, A, aW, Y, ff, r, aq, en, t, cm, bz, bN, ck, nB, dB, m, bt); #c1(RASSF7) c2(NP_001137466) c3(ras association domain-containing protein 7 isoform 3) c4(55880) c5(55881, 55882, 55883, 55884, 55885, 55886, 55887, 55888, 55889, 55890) c6( ) c7(T, pY, ko, q, er, av, aT, iv, dL, bK, eL, A, aW, Y, ff, r, aq, en, t, cm, bz, bN, ck, nB, dB, m, bt); #c1(RASSF7) c2(NP_003466) c3(ras association domain-containing protein 7 isoform 1) c4(138562) c5(138563, 138564, 138565, 138566, 138567, 138568, 138569, 138570) c6( ) c7(T, pY, ko, q, er, av, aT, iv, dL, bK, eL, A, aW, Y, ff, r, aq, en, t, cm, bz, bN, ck, nB, dB, m, bt); #c1(RASSF7) c2(XP_005253204) c3(ras association domain-containing protein 7 isoform X1) c4( ) c5(272281) c6( ) c7(T, pY, ko, q, er, av, aT, iv, dL, bK, eL, A, aW, Y, ff, r, aq, en, t, cm, bz, bN, ck, nB, dB, m, bt); #c1(RASSF7) c2(XP_005253205) c3(ras association domain-containing protein 7 isoform X2) c4( ) c5(272282, 272283, 272284) c6( ) c7(T, pY, ko, q, er, av, aT, iv, dL, bK, eL, A, aW, Y, ff, r, aq, en, t, cm, bz, bN, ck, nB, dB, m, bt); #c1(RASSF7) c2(XP_005253206) c3(ras association domain-containing protein 7 isoform X3) c4( ) c5(272285) c6( ) c7(T, pY, ko, q, er, av, aT, iv, dL, bK, eL, A, aW, Y, ff, r, aq, en, t, cm, bz, bN, ck, nB, dB, m, bt); #c1(RASSF7) c2(XP_006718401) c3(ras association domain-containing protein 7 isoform X6) c4(291823) c5( ) c6( ) c7(T, pY, ko, q, er, av, aT, iv, dL, bK, eL, A, aW, Y, ff, r, aq, en, t, cm, bz, bN, ck, nB, dB, m, bt); #c1(RASSF7) c2(XP_006725207) c3(ras association domain-containing protein 7 isoform X7) c4( ) c5(296718) c6( ) c7(T, pY, ko, q, er, av, aT, iv, dL, bK, eL, A, aW, Y, ff, r, aq, en, t, cm, bz, bN, ck, nB, dB, m, bt); #c1(RASSF7) c2(XP_011518690) c3(ras association domain-containing protein 7 isoform X4) c4( ) c5(313077) c6( ) c7(T, pY, ko, q, er, av, aT, iv, dL, bK, eL, A, aW, Y, ff, r, aq, en, t, cm, bz, bN, ck, nB, dB, m, bt); #c1(RASSF8) c2(NP_001158219) c3(ras association domain-containing protein 8 isoform a) c4(63416, 63417) c5(63418, 63419, 63420, 63421, 63422, 63423, 63424, 63425, 63426, 63427, 63428, 63429, 63430, 63431, 63432, 63433, 63434, 63435) c6( ) c7(av, ft, aHC, g, bz, fq, iu); #c1(RASSF8) c2(NP_009142) c3(ras association domain-containing protein 8 isoform b) c4(171069, 171070, 171071, 171072, 171073, 171074, 171075) c5(171076, 171077, 171078, 171079, 171080, 171081, 171082, 171083, 171084, 171085, 171086, 171087, 171088, 171089, 171090, 171091, 171092, 171093, 171094, 171095, 171096, 171097, 171098, 171099, 171100, 171101, 171102, 171103, 171104, 171105, 171106, 171107, 171108, 171109, 171110, 171111, 171112, 171113, 171114, 171115, 171116, 171117, 171118, 171119, 171120, 171121, 171122, 171123) c6( ) c7(av, ft, aHC, g, bz, fq, iu); #c1(RAX) c2(NP_038463) c3(retinal homeobox protein Rx) c4(176533, 176534) c5(176535, 176536, 176537, 176538, 176539, 176540, 176541, 176542, 176543, 176544, 176545, 176546, 176547, 176548) c6( ) c7(kn, kj, cyz, Y, bYC, Az, lo, K, afu); #c1(RAX2) c2(NP_116142) c3(retina and anterior neural fold homeobox protein 2) c4( ) c5(225480, 225481, 225482, 225483, 225484) c6( ) c7(cyB, nw, cyA); #c1(RAX2) c2(XP_005259719) c3(retina and anterior neural fold homeobox protein 2 isoform X1) c4(276247, 276248, 276249) c5(276250, 276251, 276252, 276253, 276254, 276255, 276256, 276257, 276258, 276259, 276260, 276261, 276262, 276263) c6( ) c7(cyB, nw, cyA); #c1(RBI) c2(NP_000312) c3(retinoblastoma-associated protein) c4(6942, 6943, 6944) c5(6945, 6946, 6947, 6948, 6949, 6950, 6951, 6952, 6953, 6954, 6955, 6956, 6957, 6958, 6959, 6960, 6961, 6962) c6( ) c7(G, T, aC, fR, hV, ea, if, al, WV, fd, bB, ci, YD, ip, x, bv, aE, pX, Qk, avA, SV, iv, hu, eL, aJE, xQ, eG, yo, zQ, aW, g, as, eF, cm, bw, BY, eM, nB, AM, iA, iT, aep, cj, zv, k, m, Xu, dD, aEj, wu, ht, qy, n, fr, qN, zP, cde, BI, ft, zf, By, dL, ey, Dz, YT, dx, YI, AQ, kp, Hu, Qy, YE, bP, dB, hk, hL, hj, hR, E, MW, it, B, cn, fM, jp, jx, aA, hy, v, bZP, q, er, eA, XX, mj, Yn, lg, hW, qK, aT, aHs, j, an, ae, af, eu, jf, qw, s, vT, iU, Y, r, en, cl, t, ck, QG, iM, au, aor, cdb, bt, On, Gl, kh, nl, id, eP, ow, aD, iW, jh, Hn, bnS, Fb, eq, hF, iJ, ad, dP, av, bog, hP, wm, iL, d, BN, je, qt, ff, wc, eH, aF, bz, dr, bF, es, gk, amS, bE, bC, y, kS); #c1(RBI) c2(XP_011533473) c3(retinoblastoma-associated protein isoform X1) c4( ) c5(329581) c6( ) c7(G, T, aC, fR, hV, ea, if, al, WV, fd, bB, ci, YD, ip, x, bv, aE, pX, Qk, avA, SV, iv, hu, eL, aJE, xQ, eG, yo, zL, aW, g, as, eF, cm, bw, eY, eM, nB, AM, iA, iT, aep, cj, zv, k, m, Xu, dD, aEj, wu, ht, qy, n, fr, qN, zP, cde, BI, ft, zf, By, dL, ey, Dz, YT, dx, YI, AQ, kp, Hu, By, YE, bP, dB, hk, hl, hj, hR, E, MW, it, B, cn, fM, jp, jx, aA, hy, v, bZP, q, er, eA, XX, mj, Yn, lg, hW, qK, aT, aHs, j, an, ae, af, eu, jf, qw, s, vT, iU, Y, r, en, cl, t, ck, QG, iM, au, aor, cdb, bt, On, Gl, kh, nl, id, eP, ow, aD, iW, jh, Hn, bnS, Fb, eq, hF, iJ, ad, dP, av, bog, hP, wm, iL, d, BN, je, qt, ff, wc, eH, aF, bz, dr, bF, es, gk, amS, bE, bC, y, kS); #c1(RBICC1) c2(NP_001077086) c3(RBI-inducible coiled-coil protein 1 isoform 2) c4(37715) c5(37716, 37717, 37718, 37719, 37720, 37721, 37722, 37723, 37724, 37725) c6( ) c7(bF, T, d, bP, aW, eH, aT, at, dL, K, im, eF, ae, X, ad); #c1(RBICC1) c2(NP_055596) c3(RBI-inducible coiled-coil protein 1 isoform 1) c4(182160, 182161, 182162) c5(182163, 182164, 182165, 182166, 182167, 182168, 182169, 182170, 182171, 182172, 182173, 182174) c6(182175, 182176) c7(bF, T, d, bP, aW, eH, aT, at, dL, K, im, eF, ae, X, ad); #c1(RBICC1) c2(XP_011515947) c3(RBI-inducible coiled-coil protein 1 isoform X3) c4(309088) c5(309089) c6( ) c7(bF, T, d, bP, aW, eH, aT, at, dL, K, im, eF, ae, X, ad); #c1(RBICC1) c2(XP_011515950) c3(RBI-inducible coiled-coil protein 1 isoform X6) c4(309090) c5( ) c6( ) c7(bF, T, d, bP, aW, eH, aT, at, dL, K, im, eF, ae, X, ad); #c1(RBICC1) c2(XP_011515951) c3(RBI-inducible coiled-coil protein 1 isoform X7) c4( ) c5(309091, 309092) c6( ) c7(bF, T, d, bP, aW, eH, aT, at, dL, K, im, eF, ae, X, ad); #c1(RBAK) c2(NP_066986) c3(RB-associated KRAB zinc finger protein) c4(208025, 208026) c5(208027, 208028, 208029, 208030, 208031, 208032, 208033, 208034, 208035, 208036, 208037, 208038, 208039, 208040, 208041, 208042) c6( ) c7(Ea, oe, agv, fq); #c1(RBBP5) c2(NP_001180201) c3(retinoblastoma-binding protein 5 isoform 2) c4(73370) c5(73371, 73372, 73373, 73374, 73375, 73376) c6( ) c7(d, ac); #c1(RBBP5) c2(NP_005048) c3(retinoblastoma-binding protein 5 isoform 1) c4(152440) c5(152441, 152442, 152443, 152444, 152445, 152446, 152447) c6( ) c7(d, ac); #c1(RBBP6) c2(NP_008841) c3(E3 ubiquitin-protein ligase RBBP6 isoform 1) c4(168977, 168978) c5( ) c6( ) c7(av, T, eW, ac, bP, iE, bE, aW, ff, ht, aT, bB, aj, ci, en, hu, g, bz, eA, J); #c1(RBBP6) c2(NP_061173) c3(E3 ubiquitin-protein ligase RBBP6 isoform 2) c4(199754) c5(199755, 199756, 199757, 199758, 199759, 199760) c6( ) c7(av, T, eW, ac, bP, iE, bE, aW, ff, ht, aT, bB, aj, ci, en, hu, g, bz, eA, J); #c1(RBBP6) c2(NP_116015) c3(E3 ubiquitin-protein ligase RBBP6 isoform 3) c4(225213) c5(225214, 225215, 225216, 225217, 225218, 225219) c6( ) c7(av, T, eW, ac, bP, iE, bE, aW, ff, ht, aT, bB, aj, ci, en, hu, g, bz, eA, J); #c1(RBBP6) c2(XP_005255519) c3(E3 ubiquitin-protein ligase RBBP6 isoform X2) c4( ) c5(273700, 273701, 273702, 273703, 273704, 273705, 273706, 273707, 273708, 273709, 273710, 273711) c6( ) c7(av, T, eW, ac, bP, iE, bE, aW, ff, ht, aT, bB, aj, ci, en, hu, g, bz, eA, J); #c1(RBBP7) c2(NP_001185648) c3(histone-binding protein RBBP7 isoform 1) c4(76023) c5(76024, 76025, 76026, 76027, 76028, 76029) c6( ) c7(bP, dB, aW, aT, nf, ci, K, er); #c1(RBBP7) c2(NP_002884) c3(histone-binding protein RBBP7 isoform 2) c4(133180, 133181) c5(133182, 133183, 133184, 133185, 133186, 133187, 133188, 133189, 133190, 133191, 133192, 133193) c6( ) c7(bP, dB, aW, aT, nf, ci, K, er); #c1(RBBP8) c2(NP_976037) c3(DNA endonuclease RBBP8 isoform b) c4(262851, 262852) c5(262853, 262854, 262855, 262856, 262857, 262858, 262859, 262860, 262861, 262862, 262863, 262864) c6( ) c7(T, aC, fR, ci, iJ, q, ad, aE, aT, dL, K, ae, eL, aW, WE, r, g, cyC, t, and, bP, coK, m, us, ahZ); #c1(RBBP8) c2(XP_005258383) c3(DNA endonuclease RBBP8 isoform X3) c4( ) c5(275641) c6( ) c7(T, aC, fR, ci, iJ, q, ad, aE, aT, dL, K, ae, eL, aW, WE, r, g, cyC, t, aQd, bP, coK, m, us, ahZ); #c1(RBBP8) c2(XP_006722584) c3(DNA endonuclease RBBP8 isoform X1) c4(294739, 294740) c5(294741, 294742, 294743, 294744, 294745, 294746, 294747, 294748, 294749, 294750, 294751, 294752, 294753, 294754, 294755, 294756) c6( ) c7(T, aC, fR, ci, iJ, q, ad, aE, aT, dL, K, ae, eL, aW, WE, r, g, cyC, t, aQd, bP, coK, m, us, ahZ); #c1(RBBP9) c2(NP_006597) c3(putative hydrolase RBBP9) c4(166513, 166514) c5(166515, 166516, 166517, 166518, 166519, 166520, 166521, 166522, 166523, 166524, 166525, 166526, 166527) c6( ) c7(bP, ci); #c1(RBCK1) c2(NP_006453) c3(ranBP-type and C3HC4-type zinc finger-containing protein 1 isoform 1) c4(165347) c5(165348, 165349, 165350, 165351, 165352, 165353) c6( ) c7(cc, yQ, aW, oj, aT, cbL); #c1(RBCK1) c2(NP_112506) c3(ranBP-type and C3HC4-type zinc finger-containing protein 1 isoform 2) c4(221284, 221285, 221286, 221287) c5(221288, 221289, 221290, 221291, 221292, 221293, 221294, 221295, 221296, 221297, 221298, 221299, 221300, 221301, 221302, 221303, 221304, 221305, 221306, 221307, 221308, 221309, 221310, 221311, 221312, 221313, 221314, 221315) c6( ) c7(cc, yQ, aW, oj, aT, cbL); #c1(RBCK1) c2(XP_011527440) c3(ranBP-type and C3HC4-type zinc finger-containing protein 1 isoform X2) c4(322185) c5(322186, 322187, 322188, 322189, 322190, 322191) c6( ) c7(cc, yQ, aW, oj, aT, cbL); #c1(RBCK1) c2(XP_011527441) c3(ranBP-type and C3HC4-type zinc finger-containing protein 1 isoform X4) c4(322192) c5( ) c6( ) c7(cc, yQ, aW, oj, al, cbL); #c1(RBCK1) c2(XP_011527442) c3(ranBP-type and C3HC4-type zinc finger-containing protein 1 isoform X5) c4( ) c5(322193, 322194, 322195, 322196, 322197, 322198) c6( ) c7(cc, yQ, aW, oj, aT, cbL); #c1(RBFOX1) c2(NP_001135805) c3(RNA binding protein fox-1 homolog 1 isoform 5) c4(53288) c5(53289, 53290, 53291, 53292, 53293, 53294) c6( ) c7(Cq, rq, c, af, rz, Gp, wL, ha, r, akf, fe, avl, rs, j, bK, rx, v, aDm, Bd, q, bN); #c1(RBFOX1) c2(NP_00135806) c3(RNA binding protein fox-1 homolog 1 isoform 4) c4(53295) c5(53296, 53297, 53298, 53299, 53300, 53301, 53302, 53303, 53304, 53305, 53306, 53307, 53308, 53309) c6( ) c7(Cq, rq, c, af, rz, Gp, wL, ha, r, akf, fe, avl, rs, j, bK, rx, v, aDm, Bd, q, bN); #c1(RBFOX1) c2(NP_665898) c3(RNA binding protein fox-1 homolog 1 isoform 1) c4(241925) c5(241926, 241927, 241928, 241929, 241930, 241931) c6( ) c7(Cq, rq, c, af, rz, Gp, wL, ha, r, akf, fe, avl, rs, j, bK, rx, v, aDm, Bd, q, bN); #c1(RBFOX1) c2(NP_665899) c3 (RNA binding protein fox-1 homolog 1 isoform 2) c4(241932) c5(241933, 241934, 241935, 241936, 241937, 241938) c6( ) c7(Cq, rq, c, af, rz, Gp, wL, ha, r, akf, fe, avl, rs, j, bK, rx, v, aDm, Bd, q, bN); #c1(RBFOX1) c2(NP_665900) c3(RNA binding protein fox-1 homolog 1 isoform 3) c4( ) c5(241939, 241940, 241941, 241942, 241943, 241944) c6( ) c7(Cq, rq, c, af, rz, Gp, wL, ha, r, akf, fe, avl, rs, j, bK, rx, v, aDm, Bd, q, bN); #c1(RBFOX1) c2(XP_005255436) c3(RNA binding protein fox-1 homolog 1 isoform X2) c4(273657) c5(273658, 273659, 273660, 273661, 273662, 273663) c6( ) c7(Cq, rq, c, af, rz, Gp, wL, ha, r, akf, fe, avl, rs, j, bK, rx, v, aDm, Bd, q, bN); #c1(RBFOX1) c2(XP_005255444) c3(RNA binding protein fox-1 homolog 1 isoform X10) c4(273664) c5( ) c6( ) c7(Cq, rq, c, af, rz, Gp, wL, ha, r, akf, fe, avl, rs, j, bK, rx, v, aDm, Bd, q, bN); #c1(RBFOX1) c2(XP_005255451) c3(RNA binding protein fox-1 homolog 1 isoform X17) c4(273665) c5(273666, 273667, 273668, 273669, 273670, 273671) c6( ) c7(Cq, rq, c, af, rz, Gp, wL, ha, r, akf, fe, avl, rs, j, bK, rx, v, aDm, Bd, q, bN); #c1(RBFOX1) c2(XP_011520848) c3(RNA binding protein fox-1 homolog 1 isoform X14) c4(315831) c5(315832, 315833, 315834, 315835, 315836, 315837) c6( ) c7(Cq, rq, c, af, rz, Gp, wL, ha, r, akf, fe, avl, rs, j, bK, rx, v, aDm, Bd, q, bN); #c1(RBFOX2) c2(NP_001026865) c3(RNA binding protein fox-1 homolog 2 isoform 1) c4(29678) c5(29679, 29680, 29681, 29682, 29683, 29684) c6( ) c7(T, eR, zs, ha, bB, lR, nA, ad, fU, TI, qK, aT, h, ajT, aW, aq, lo, yq, iR, akf, t, bz, pb, ck, bE, NL, m, bt); #c1(RBFOX2) c2(NP_001076045) c3(RNA binding protein fox-1 homolog 2 isoform 3) c4(37343, 37344) c5(37345, 37346, 37347, 37348, 37349, 37350, 37351, 37352, 37353, 37354, 37355, 37356) c6( ) c7(T, eR, zs, ha, bB, lR, nA, ad, fU, TI, qK, aT, h, ajT, aW, aq, lo, yq, iR, akf, t, bz, pb, ck, bE, NL, m, bt); #c1(RBFOX2) c2(NP_001076046) c3(RNA binding protein fox-1 homolog 2 isoform 4) c4(37357) c5(37358, 37359, 37360, 37361, 37362, 37363, 37364, 37365, 37366, 37367, 37368, 37369) c6( ) c7(T, eR, zs, ha, bB, lR, nA, ad, fU, TI, qK, aT, h, ajT, aW, aq, lo, yq, iR, akf, t, bz, pb, ck, bE, NL, m, bt); #c1(RBFOX2) c2(NP_001076047) c3(RNA binding protein fox-1 homolog 2 isoform 5) c4(37370) c5(37371, 37372, 37373, 37374, 37375, 37376) c6( ) c7(T, eR, zs, ha, bB, lR, nA, ad, fU, TI, qK, aT, h, ajT, aW, aq, lo, yq, iR, akf, t, bz, pb, ck, bE, NL, m, bt); #c1(RBFOX2) c2(NP_055124) c3(RNA binding protein fox-1 homolog 2 isoform 2) c4(179098) c5(179099, 179100, 179101, 179102, 179103, 179104) c6( ) c7(T, eR, zs, ha, bB, lR, nA, ad, fU, TI, qK, aT, h, ajT, aW, aq, lo, yq, iR, akf, t, bz, pb, ck, bE, NL, m, bt); #c1(RBFOX2) c2(XP_005261486) c3(RNA binding protein fox-1 homolog 2 isoform X4) c4(277375, 277376) c5(277377, 277378, 277379, 277380, 277381, 277382, 277383, 277384, 277385, 277386, 277387, 277388) c6( ) c7(T, eR, zs, ha, bB, lR, nA, ad, fU, TI, qK, aT, h, ajT, aW, aq, lo, yq, iR, akf, t, bz, pb, ck, bE, NL, m, bt); #c1(RBFOX2) c2(XP_005261488) c3(RNA binding protein fox-1 homolog 2 isoform X7) c4(277389) c5(277390, 277391, 277392, 277393, 277394, 277395) c6( ) c7(T, eR, zs, ha, bB, lR, nA, ad, fU, TI, qK, aT, h, ajT, aW, aq, lo, yq, iR, akf, t, bz, pb, ck, bE, NL, m, bt); #c1(RBFOX2) c2(XP_005261492) c3(RNA binding protein fox-1 homolog 2 isoform X15) c4(277396) c5( ) c6( ) c7(T, eR, zs, ha, bB, lR, nA, ad, fU, TI, qK, aT, h, ajT, aW, aq, lo, yq, iR, akf, t, bz, pb, ck, bE, NL, m, bt); #c1(RBFOX2) c2(XP_006724252) c3(RNA binding protein fox-1 homolog 2 isoform X11) c4(295881) c5(295882, 295883, 295884, 295885, 295886, 295887) c6( ) c7(T, eR, zs, ha, bB, lR, nA, ad, fU, TI, qK, aT, h, ajT, aW, aq, lo, yq, iR, akf, t, bz, pb, ck, bE, NL, m, bt); #c1(RBFOX3) c2(NP_001076044) c3(RNA binding protein fox-1 homolog 3) c4(37332) c5(37333, 37334, 37335, 37336, 37337, 37338, 37339, 37340, 37341, 37342) c6( ) c7(rR, c, ha, eZ, Lp, cy, avl, Kh, K, fL, an, akf, on, mC); #c1(RBL1) c2(NP_002886) c3(retinoblastoma-like protein 1isoform a) c4(133194) c5(133195, 133196, 133197, 133198, 133199, 133200, 133201) c6( ) c7(ae, T, ft, av, bP, s, Db, aW, if, al, n, ci, qN, cl, t, bz, ad); #c1(RBL1) c2(NP_899662) c3(retinoblastoma-like protein 1 isoform b) c4(258089) c5(258090, 258091, 258092, 258093, 258094, 258095) c6( ) c7(ae, T, ft, av, bP, s, Db, aW, if, aT, n, ci, qN, cl, t, bz, ad); #c1(RBL2) c2(XP_005256140) c3(retinoblastoma-like protein 2 isoform X2) c4(274004) c5(274005, 274006, 274007, 274008, 274009, 274010, 274011) c6( ) c7(T, if, iW, jh, aA, n, ci, qN, q, ad, eA, av, ft, ib, lg, aT, pk, ae, af, aJE, d, aW, ff, eH, r, hQ, aF, en, cI, eF, t, cm, bz, bP, eD, hL, cj, m, it, B, L); #c1(RBL2) c2(XP_011521554) c3(retinoblastoma-like protein 2 isoform X1) c4(316251) c5(316252, 316253, 316254, 316255, 316256, 316257, 316258) c6( ) c7(T, if, iW, jh, aA, n, ci, qN, q, ad, eA, av, ft, ib, lg, aT, pk, ae, af, aJE, d, aW, ff, eH, r, hQ, aF, en, cl, eF, t, cm, bz, bP, eD, hL, cj, m, it, B, L); #c1(RBL2) c2(XP_011521555) c3(retinoblastoma-like protein 2 isoform X3) c4(316259, 316260) c5(316261, 316262, 316263, 316264, 316265, 316266, 316267, 316268, 316269, 316270, 316271) c6( ) c7(T, if, iW, jh, aA, n, ci, qN, q, ad, eA, av, ft, ib, lg, aT, pk, ae, af, aJE, d, aW, ff, eL, r, hQ, aF, en, cl, eF, t, cm, bz, bP, eD, hL, cj, m, it, B, L); #c1(RBM10) c2(NP_001191397) c3(RNA-binding protein 10 isoform 5) c4( ) c5(81496) c6( ) c7(Na, T, aW, aT, bz, cyD); #c1(RBM10) c2(NP_005667) c3(RNA-binding protein 10 isoform 1) c4(158379) c5(158380, 158381, 158382, 158383, 158384) c6( ) c7(Na, T, aW, aT, bz, cyD); #c1(RBM10) c2(NP_690595) c3(RNA-binding protein 10 isoform 2) c4(244884) c5(244885, 244886, 244887, 244888, 244889) c6( ) c7(Na, T, aW, aT, bz, cyD); #c1(RBM10) c2(XP_005272735) c3(RNA-binding protein 10 isoform X2) c4(284168) c5(284169, 284170, 284171, 284172, 284173, 284174) c6( ) c7(Na, T, aW, aT, bz, cyD); #c1(RBM10) c2(XP_005272736) c3(RNA-binding protein 10 isoform X3) c4( ) c5(284175) c6( ) c7(Na, T, aW, aT, bz, cyD); #c1(RBM12) c2(NP_001185769) c3(RNA-binding protein (2) c4(76252, 76253, 76254, 76255, 76256) c5(76257, 76258, 76259, 76260, 76261, 76262, 76263, 76264, 76265, 76266, 76267, 76268, 76269, 76270, 76271, 76272, 76273, 76274, 76275, 76276, 76277, 76278, 76279, 76280, 76281, 76282, 76283, 76284, 76285, 76286, 76287, 76288, 76289, 76290, 76291, 76292, 76293, 76294) c6( ) c7(PE); #c1(RBM14) c2(NP_001185765) c3(RNA-binding protein 14 isoform 2) c4(76238) c5(76239, 76240, 76241, 76242, 76243, 76244) c6( ) c7(T, cc, d, aE, eM, iA, aW, jh, aT, aA, n, aF, fr, ci, eA); #c1 (RBM14) c2(NP_001185766) c3(RNA-binding protein 14 isoform 3) c4(76245) c5(76246, 76247, 76248, 76249, 76250, 76251) c6( ) c7(T, cc, d, aE, eM, iA, aW, jh, aT, aA, n, aF, fr, ci, eA); #c1(RBM14) c2(NP_006319) c3(RNA-binding protein 14 isoform 1) c4(163999) c5(164000, 164001, 164002, 164003, 164004, 164005) c6( ) c7(T, cc, d, aE, eM, iA, aW, jh, aT, aA, n, aF, fr, ci, eA); #c1(RBM14-RBM4) c2(NP_001185774) c3(RBM14-RBM4 protein isoform 1) c4(76319) c5(76320, 76321, 76322, 76323, 76324, 76325) c6( ) c7(T, cc, d, aE, eM, iA, aW, jh, aA, aT, n, aF, fr, eA); #c1(RBM14-RBM4) c2(NP_001185775) c3(RBM14-RBM4 protein isoform 2) c4(76326) c5(76327, 76328, 76329, 76330, 76331, 76332) c6( ) c7(T, cc, d, aE, eM, iA, aW, jh, aA, aT, n, aF, fr, eA); #c1(RBM15) c2(XP_011540270) c3(putative RNA-binding protein 15 isoform X2) c4(338681) c5(338682, 338683, 338684, 338685, 338686, 338687) c6( ) c7(ck, T, iU, iY, f, bt); #c1(RBM17) c2(NP_001139019) c3(splicing factor 45) c4(58625, 58626, 58627, 58628, 58629) c5(58630, 58631, 58632, 58633, 58634, 58635, 58636, 58637, 58638, 58639, 58640, 58641, 58642, 58643, 58644, 58645, 58646, 58647, 58648, 58649, 58650, 58651, 58652, 58653, 58654, 58655, 58656, 58657, 58658, 58659, 58660, 58661, 58662, 58663, 58664, 58665, 58666, 58667) c6( ) c7(T, A, kB, Kh, ci, qN); #c1(RBM20) c2(NP_001127835) c3(RNA-binding protein 20) c4(49287, 49288) c5(49289, 49290, 49291, 49292, 49293, 49294, 49295, 49296, 49297, 49298, 49299, 49300) c6( ) c7(cc, anR, oe, mx, tc, cyE, cd); #c1(RBM25) c2(XP_011535346) c3(RNA-binding protein 25 isoform X1) c4(332337, 332338, 332339, 332340, 332341) c5(332342, 332343, 332344, 332345, 332346, 332347, 332348, 332349, 332350, 332351, 332352, 332353, 332354, 332355, 332356, 332357, 332358, 332359, 332360, 332361, 332362, 332363, 332364, 332365, 332366, 332367, 332368, 332369) c6( ) c7(cd); #c1(RBM25) c2(XP_011535347) c3(RNA-binding protein 25 isoform X2) c4( ) c5(332370, 332371, 332372, 332373, 332374, 332375, 332376, 332377, 332378, 332379) c6( ) c7(cd); #c1(RBM26) c2(NP_001273561) c3(RNA-binding protein 26 isoform 2) c4(105234) c5(105235, 105236, 105237, 105238, 105239, 105240) c6( ) c7(jh); #c1(RBM26) c2(NP_071401) c3(RNA-binding protein 26 isoform 3) c4(211436) c5(211437, 211438, 211439, 211440, 211441, 211442) c6( ) c7(jh); #c1(RBM26) c2(XP_011533490) c3(RNA-binding protein 26 isoform X12) c4(329582) c5(329583, 329584, 329585, 329586, 329587, 329588) c6( ) c7(jh); #c1(RBM27) c2(NP_061862) c3(RNA-binding protein 27) c4(200840) c5(200841, 200842, 200843, 200844, 200845, 200846) c6( ) c7(aid); #c1(RBM28) c2(NP_001159607) c3(RNA-binding protein 28 isoform 2) c4(64463) c5(64464, 64465, 64466, 64467, 64468, 64469) c6( ) c7(cyF, bHF, ml); #c1(RBM28) c2(NP_060547) c3(RNA-binding protein 28 isoform 1) c4(196508) c5(196509, 196510, 196511, 196512, 196513, 196514) c6( ) c7(cyF, bHF, ml); #c1(RBM28) c2(XP_011514673) c3(RNA-binding protein 28 isoform X2) c4( ) c5(307328) c6( ) c7(cyF, bHF, ml); #c1(RBM3) c2(NP_006734) c3(RNA-binding protein 3) c4(167554) c5(167555, 167556, 167557, 167558, 167559, 167560, 167561, 167562, 167563, 167564, 167565) c6( ) c7(T, cyG, GU, aW, aT, r, n, K, ae, q, ad); #c1(RBM3) c2(XP_011542240) c3(RNA-binding protein 3 isoform X1) c4(340739, 340740) c5(340741, 340742, 340743, 340744, 340745, 340746, 340747, 340748, 340749, 340750, 340751, 340752, 340753, 340754) c6( ) c7(T, cyG, GU, aW, aT, r, n, K, ae, q, ad); #c1(RBM38) c2(NP_001278709) c3(RNA-binding protein 38 isoform c) c4(110003) c5(110004) c6( ) c7(T, aW, aw, jh, aT, K, m, t); #c1(RBM38) c2(NP_059965) c3(RNA-binding protein 38 isoform a) c4(193410, 193411) c5(193412, 193413, 193414, 193415, 193416, 193417, 193418, 193419, 193420, 193421, 193422) c6( ) c7(T, aW, aw, jh, aT, K, m, t); #c1(RBM38) c2(NP_906270) c3(RNA-binding protein 38 isoform b) c4( ) c5(258103, 258104, 258105, 258106, 258107, 258108) c6( ) c7(T, aW, aw, jh, aT, K, m, t); #c1(RBM39) c2(NP_001229528) c3(RNA-binding protein 39 isoform c) c4(84603, 84604) c5(84605, 84606, 84607, 84608, 84609, 84610, 84611, 84612, 84613, 84614, 84615, 84616) c6( ) c7(gk, T, eJ, eM, eR, aW, al, aT, hF, bz, B, hX); #c1(RBM39) c2(NP_004893) c3(RNA-binding protein 39 isoform b) c4(150615, 150616, 150617) c5(150618, 150619, 150620, 150621, 150622, 150623, 150624, 150625, 150626, 150627, 150628, 150629) c6( ) c7(gk, T, eJ, eM, eR, aW, al, aT, hF, bz, B, hX); #c1(RBM39) c2(NP_909122) c3(RNA-binding protein 39 isoform a) c4(258219, 258220, 258221) c5(258222, 258223, 258224, 258225, 258226, 258227, 258228) c6( ) c7(gk, T, eJ, eM, eR, aW, al, aT, hF, bz, B, hX); #c1(RBM39) c2(XP_006723956) c3(RNA-binding protein 39 isoform X2) c4( ) c5(295574, 295575, 295576, 295577, 295578) c6( ) c7(gk, T, eJ, eM, eR, aW, al, aT, hF, bz, B, hX); #c1(RBM39) c2(XP_011527412) c3(RNA-binding protein 39 isoform X1) c4( ) c5(322144, 322145, 322146, 322147, 322148, 322149, 322150, 322151, 322152, 322153, 322154, 322155) c6( ) c7(gk, T, eJ, eM, eR, aW, al, aT, hF, bz, B, hX); #c1(RBM4) c2(NP_001185772) c3(RNA-binding protein 4 isoform 2) c4(76295) c5(76296, 76297, 76298, 76299, 76300, 76301, 76302, 76303, 76304, 76305, 76306, 76307, 76308, 76309, 76310, 7631l) c6( ) c7(f, aW, Hx, aT); #c1(RBM4) c2(NP_001185773) c3(RNA-binding protein 4 isoform 3) c4(76312) c5(76313, 76314, 76315, 76316, 76317, 76318) c6( ) c7(f, aW, Hx, aT); #c1(RBM4) c2(NP_002887) c3(RNA-binding protein 4 isoform 1) c4(133202, 133203) c5(133204, 133205, 133206, 133207, 133208, 133209, 133210, 133211, 133212, 133213, 133214, 133215, 133216, 133217, 133218, 133219, 133220, 133221, 133222, 133223, 133224, 133225) c6( ) c7(f, aW, Hx, aT); #c1(RBM45) c2(NP_694453) c3(RNA-binding protein 45) c4(245071, 245072) c5(245073, 245074, 245075, 245076, 245077, 245078, 245079, 245080, 245081, 245082, 245083, 245084) c6( ) c7(ID, bM, T, bJN, xS, bJx, if, e, al, fd, aeu, cL, bwS, qj, ci, bh, aFV, Jx, Jt, bJe, aY, Tz, lR, aMB, ip, NF, x, AW, xy, xE, vV, aE, bW, dV, azm, rB, K, aFk, bc, azh, aEV, JI, aW, axu, lh, HA, hl, kt, hN, aVO, pL, bJy, bw, y, xK, Jr, lp, ii, blQ, mx, cj, dW, bAD, k, m, lS, abh, Ro, xN, aYZ, yG, fN, ez, n, cQ, aj, fr, uE, pM, qN, apz, qW, cd, br, ai, cde, bJf, ke, Mo, cc, KN, aMj, h, ey, alY, nP, OV, mR, A, bJl, sp, lb, fe, ax, CQ, mo, gG, Ld, oU, ar, W, bP, lX, bbx, aZL, lf, bm, awQ, bzm, eo, ase, acu, Pr, B, abW, bJp, bL, eR, jx, eX, aA, aBo, j, Vh, V, pd, eA, J, o, MK, cF, fh, akU, qp, ld, bJE, aT, vH, KU, lM, sX, Cy, ae, af, dL, aAi, aFS, kl, hM, iU, xR, Y, ayU, lC, Pg, aMA, lo, bJC, aDm, t, pb, bN, lY, ck, cT, ahN, nM, ac, iM, hA, ig, Zg, QG, mG, of, AH, uS, aYx, Da, dy, oX, fD, bt, blu, vi, Kd, bJq, ow, aD, axd, aZU, dG, uu, jE, hF, aYs, Uy, aue, XB, BY, qC, aoH, ad, hX, F, wD, oz, Dq, ds, bJL, alg, qh, oj, LA, dm, hD, JK, vB, ji, vO, yF, uQ, atJ, je, lR, aFE, aGS, OQ, azP, wc, cr, eZ, aF, hp, afi, iV, bz, aYt, bJt, LB, ay, gk, Bv, amS, atM, z, Rv, gy, ME, rH, bJo, oQ, ei, hE, ra, hv, pC); #c1(RBM46) c2(NP_659416) c3(probable RNA-binding protein 46 isoform 1) c4(239792) c5(239793, 239794, 239795, 239796, 239797, 239798) c6( ) c7(sj); #c1(RBM47) c2(NP_001092104) c3(RNA-binding protein 47 isoform a) c4(39377) c5(39378, 39379, 39380, 39381, 39382, 39383, 39384, 39385, 39386, 39387) c6( ) c7(aj, j); #c1(RBM47) c2(NP_061900) c3(RNA-binding protein 47 isoform b) c4(201063, 201064, 201065, 201066, 201067, 201068, 201069, 201070) c5(201071, 201072, 201073, 201074, 201075, 201076, 201077, 201078, 201079, 201080, 201081, 201082, 201083, 201084, 201085, 201086, 201087, 201088, 201089, 201090, 201091, 201092, 201093, 201094, 201095, 201096, 201097, 201098, 201099, 201100, 201101, 201102, 201103, 201104, 201105, 201106, 201107, 201108, 201109, 201110, 201111, 201112, 201113, 201114, 201115, 201116, 201117, 201118, 201119, 201120, 201121, 201122, 201123, 201124, 201125, 201126) c6( ) c7(aj, j); #c1(RBM5) c2(NP_005769) c3(RNA-binding protein 5) c4(159196, 159197, 159198, 159199) c5(159200, 159201, 159202, 159203, 159204, 159205, 159206, 159207, 159208, 159209, 159210, 159211, 159212, 159213) c6( ) c7(av, T, ft, bev, aE, eM, aW, aT, vH, ci, g, bz, iu); #c1(RBM5) c2(XP_006712980) c3(RNA-binding protein 5 isoform X1) c4(286943, 286944, 286945) c5( ) c6( ) c7(av, T, ft, bev, aE, eM, aW, aT, vH, ci, g, bz, iu); #c1(RBM5) c2(XP_006712982) c3(RNA-binding protein 5 isoform X4) c4(286946) c5(286947, 286948, 286949, 286950, 286951, 286952) c6( ) c7(av, T, ft, bev, aE, eM, aW, aT, vH, ci, g, bz, iu); #c1(RBM5) c2(XP_011531563) c3(RNA-binding protein 5 isoform X2) c4(327674) c5( ) c6( ) c7(av, T, ft, bev, aE, eM, aW, aT, vH, ci, g, bz, iu); #c1(RBM5) c2(XP_011531564) c3(RNA-binding protein 5 isoform X3) c4( ) c5(327675, 327676, 327677, 327678, 327679, 327680, 327681, 327682, 327683, 327684, 327685, 327686, 327687, 327688, 327689, 327690, 327691, 327692, 327693, 327694, 327695, 327696, 327697, 327698, 327699, 327700, 327701, 327702, 327703, 327704, 327705, 327706, 327707, 327708) c6( ) c7(av, T, ft, bev, aE, eM, aW, aT, vH, ci, g, bz, iu); #c1(RBM6) c2(NP_005768) c3(RNA-binding protein 6 isoform 1) c4(159167, 159168, 159169, 159170) c5(159171, 159172, 159173, 159174, 159175, 159176, 159177, 159178, 159179, 159180, 159181, 159182, 159183, 159184, 159185, 159186, 159187, 159188, 159189, 159190, 159191, 159192, 159193, 159194, 159195) c6( ) c7(ck, T, bev, ft, iY, v, bz); #c1(RBM6) c2(XP_005264842) c3(RNA-binding protein 6 isoform X3) c4(279269) c5(279270, 279271) c6( ) c7(ck, T, bev, ft, iY, v, bz); #c1(RBM6) c2(XP_005264845) c3(RNA-binding protein 6 isoform X1) c4( ) c5(279272, 279273, 279274, 279275, 279276, 279277, 279278, 279279, 279280, 279281, 279282, 279283, 279284, 279285) c6( ) c7(ck, T, bev, ft, iY, v, bz); #c1(RBM6) c2(XP_006712979) c3(RNA-binding protein 6 isoform X2) c4(286936, 286937) c5(286938, 286939, 286940, 286941, 286942) c6( ) c7(ck, T, bev, ft, iY, v, bz); #c1(RBM7) c2(NP_057174) c3(RNA-binding protein 7 isoform b) c4(189312) c5(189313, 189314, 189315, 189316, 189317, 189318) c6( ) c7(eM); #c1(RBMS1) c2(NP_002888) c3(RNA-binding motif, single-stranded-interacting protein 1 isoform c) c4( ) c5(133226, 133227, 133228, 133229, 133230, 133231, 133232, 133233, 133234, 133235, 133236, 133237, 133238, 133239, 133240, 133241, 133242) c6( ) c7(bL, VZ, ac, c, al, j, C, Ze, B, asE); #c1(RBMS1) c2(NP_058520) c3(RNA-binding motif, single-stranded-interacting protein 1 isoform a) c4(192779) c5(192780, 192781, 192782, 192783, 192784, 192785) c6( ) c7(bL, VZ, ac, c, al, j, C, Ze, B, asE); #c1(RBMS1) c2(XP_005246796) c3(RNA-binding motif, single-stranded-interacting protein 1 isoform X5) c4( ) c5(267429) c6( ) c7(bL, VZ, ac, c, al, j, C, Ze, B, asE); #c1(RBMS1) c2(XP_005246797) c3(RNA-binding motif, single-stranded-interacting protein 1 isoform X7) c4(267430) c5(267431, 267432, 267433, 267434, 267435, 267436) c6( ) c7(bL, VZ, ac, c, al, j, C, Ze, B, asE); #c1(RBMS1) c2(XP_006712734) c3(RNA-binding motif, single-stranded-interacting protein 1 isoform X1) c4(286818) c5( ) c6( ) c7(bL, VZ, ac, c, al, j, C, Ze, B, asE); #c1(RBMS1) c2(XP_006712737) c3(RNA-binding motif, single-stranded-interacting protein 1 isoform X9) c4(286819) c5(286820, 286821) c6( ) c7(bL, VZ, ac, c, al, j, C, Ze, B, asE); #c1(RBMS2) c2(NP_002889) c3(RNA-binding motif, single-stranded-interacting protein 2) c4(133243) c5(133244, 133245, 133246, 133247, 133248, 133249) c6( ) c7(c); #c1(RBMS3) c2(NP_001003792) c3(RNA-binding motif, single-stranded-interacting protein 3 isoform 3) c4(20117) c5(20118, 20119, 20120, 20121, 20122, 20123) c6( ) c7(bM, T, aC, oe, ha, jh, aA, bB, tc, aj, TM, Vd, ci, nA, lR, fr, hF, eA, aR, ad, hX, eW, av, ft, aE, WY, ds, SV, aT, kf, Ak, dV, h, K, an, brV, fb, ae, af, dx, iL, d, vT, aW, Y, ff, eZ, nf, aq, aaE, aF, Ku, en, iR, bz, pb, QA, bN, dR, ck, rR, kP, c, ac, bE, Qu, eM, dh, mx, U, aEs, rx, xh, arg, it, B); #c1(RBMS3) c2(NP_001003793) c3(RNA-binding motif, single-stranded-interacting protein 3 isoform 1) c4(20124) c5(20125, 20126, 20127, 20128, 20129, 20130) c6( ) c7(bM, T, aC, oe, ha, jh, aA, bB, tc, aj, TM, Vd, ci, nA, lR, fr, hF, eA, aR, ad, hX, eW, av, ft, aE, WY, ds, SV, aT, kf, Ak, dV, h, K, an, brV, fb, ae, af, dx, iL, d, vT, aW, Y, ff, eZ, nf, aq, aaE, aF, Ku, en, iR, bz, pb, QA, bN, dR, ck, rR, kP, c, ac, bE, Qu, eM, dh, mx, U, aEs, rx, xh, arg, it, B); #c1(RBMS3) c2(NP_001171182) c3(RNA-binding motif, single-stranded-interacting protein 3 isoform 5) c4(69992) c5(69993, 69994, 69995, 69996, 69997, 69998) c6( ) c7(bM, T, aC, oe, ha, jh, aA, bB, tc, aj, TM, Vd, ci, nA, lR, fr, hF, eA, aR, ad, hX, eW, av, ft, aE, WY, ds, SV, aT, kf, Ak, dV, h, K, an, brV, fb, ae, af, dx, iL, d, vT, aW, Y, ff, eZ, nf, aq, aaE, aF, Ku, en, iR, bz, pb, QA, bN, dR, ck, rR, kP, c, ac, bE, Qu, eM, dh, mx, U, aEs, rx, xh, arg, it, B); #c1(RBMS3) c2(NP_001171183) c3(RNA-binding motif, single-stranded-interacting protein 3 isoform 4) c4(69999, 70000) c5(70001, 70002, 70003, 70004, 70005, 70006, 70007, 70008, 70009, 70010, 70011, 70012) c6( ) c7(bM, T, aC, oe, ha, jh, aA, bB, tc, aj, TM, Vd, ci, nA, lR, fr, hF, eA, aR, ad, hX, eW, av, ft, aE, WY, ds, SV, aT, kf, Ak, dV, h, K, an, brV, fb, ae, af, dx, iL, d, vT, aW, Y, ff, eZ, nf, aq, aaE, aF, Ku, en, iR, bz, pb, QA, bN, dR, ck, rR, kP, c, ac, bE, Qu, eM, dh, mx, U, aEs, rx, xh, arg, it, B); #c1(RBMS3) c2(NP_055298) c3(RNA-binding motif, single-stranded-interacting protein 3 isoform 2) c4(180364) c5(180365, 180366, 180367, 180368, 180369, 180370) c6( ) c7(bM, T, aC, oe, ha, jh, aA, bB, tc, aj, TM, Vd, ci, nA, lR, fr, hF, eA, aR, ad, hX, eW, av, ft, aE, WY, ds, SV, aT, kf, Ak, dV, h, K, an, brV, fb, ae, af, dx, iL, d, vT, aW, Y, ff, eZ, nf, aq, aaE, aF, Ku, en, iR, bz, pb, QA, bN, dR, ck, rR, kP, c, ac, bE, Qu, eM, dh, mx, U, aEs, rx, xh, arg, it, B); #c1(RBMS3) c2(XP_005265117) c3(RNA-binding motif, single-stranded-interacting protein 3 isoform X1) c4(279434) c5(279435, 279436, 279437, 279438, 279439, 279440) c6( ) c7(bM, T, aC, oe, ha, jh, aA, bB, tc, aj, TM, Vd, ci, nA, lR, fr, hF, eA, aR, ad, hX, eW, av, ft, aE, WY, ds, SV, aT, kf, Ak, dV, h, K, an, brV, fb, ae, af, dx, iL, d, vT, aW, Y, ff, eZ, nf, aq, aaE, aF, Ku, en, iR, bz, pb, QA, bN, dR, ck, rR, kP, c, ac, bE, Qu, eM, dh, mx, U, aEs, rx, xh, arg, it, B); #c1(RBMX) c2(NP_001158275) c3(RNA-binding motif protein, X chromosome isoform 2) c4(63522, 63523) c5(63524, 63525, 63526, 63527, 63528, 63529, 63530, 63531, 63532, 63533, 63534, 63535) c6( ) c7(T, eM, ff, eZ, aT, vH, en); #c1(RBMX) c2(NP_002130) c3(RNA-binding motif protein, X chromosome isoform 1) c4(125629, 125630) c5(125631, 125632, 125633, 125634, 125635, 125636, 125637, 125638, 125639, 125640, 125641, 125642) c6( ) c7(T, eM, ff, eZ, aT, vH, en); #c1(RBMX2) c2(NP_057108) c3(RNA-binding motif protein, X-linked 2) c4(188931, 188932, 188933) c5(188934, 188935, 188936, 188937, 188938, 188939, 188940, 188941, 188942, 188943, 188944, 188945, 188946, 188947, 188948, 188949, 188950, 188951) c6( ) c7(if); #c1(RBMXL2) c2(NP_055284) c3(RNA-binding motif protein, X-linked-like-2) c4(180275) c5(180276, 180277, 180278, 180279, 180280, 180281) c6( ) c7(gU); #c1(RBMY1A1) c2(XP_011529738) c3(RNA-binding motif protein, Y chromosome, family 1 member A1 isoform X1) c4(325385) c5(325386, 325387, 325388, 325389, 325390, 325391, 325392, 325393, 325394, 325395, 325396) c6( ) c7(bM, T, aC, gU, ha, jh, aA, bB, tc, TM, Vd, ci, nA, lR, fr, hF, eA, aR, ad, hX, eW, ft, aE, WY, ds, SV, aT, kf, Ak, dV, vH, h, K, an, brV, fb, ae, af, dx, iL, yi, d, ND, vT, aW, Y, ff, cyH, eZ, nf, aq, aaE, aF, Ku, en, iR, bz, pb, QA, bN, dR, ck, rR, kP, c, eM, bE, Qu, dh, mx, U, aEs, rx, xh, arg, B); #c1(RBP1) c2(NP_001124464) c3(retinol-binding protein 1 isoform b) c4(49076) c5(49077, 49078, 49079, 49080, 49081, 49082) c6( ) c7(Mb, T, nx, jh, aA, Gn, ci, fr, aFJ, zP, eA, ad, Zp, aE, mP, aT, bbu, dL, Fx, K, ae, eL, zQ, s, aW, Y, ff, aq, en, BE, cI, t, nC, g, op, bP, nB, AM, PI, vA, hL, m, rA, B, ao, avi); #c1(RBP1) c2(NP_001124465) c3(retinol-binding protein 1 isoform c) c4(49083) c5(49084, 49085, 49086, 49087, 49088, 49089) c6( ) c7(Mb, T, nx, jh, aA, Gn, ci, fr, aFJ, zP, eA, ad, Zp, aE, mP, aT, bbu, dL, Fx, K, ae, eL, zQ, s, aW, Y, ff, aq, en, BE, cI, t, nC, g, op, bP, nB, AM, PI, vA, hL, m, rA, B, ao, avi); #c1(RBP1) c2(NP_002890) c3(retinol-binding protein 1 isoform a) c4(133250) c5(133251, 133252, 133253, 133254, 133255, 133256) c6( ) c7(Mb, T, nx, jh, aA, Gn, ci, fr, aFJ, zP, eA, ad, Zp, aE, mP, aT, bbu, dL, Fx, K, ae, eL, zQ, s, aW, Y, ff, aq, en, BE, cI, t, nC, g, op, bP, nB, AM, PI, vA, hL, m, rA, B, ao, avi); #c1(RBP2) c2(NP_004155) c3(retinol-binding protein 2) c4(143933, 143934, 143935) c5(143936, 143937, 143938, 143939, 143940, 143941, 143942, 143943, 143944, 143945, 143946, 143947, 143948, 143949, 143950, 143951, 143952, 143953) c6( ) c7(jf, T, bP, eM, aW, oV, aA, aF, ci, bz, B, er); #c1(RBP3) c2(NP_002891) c3(retinol-binding protein 3 precursor) c4(133257) c5(133258, 133259, 133260, 133261, 133262, 133263) c6( ) c7(bxy, YE, lR, bP, nv, dB, vz, nx, qW, ik, vy, bru, h, cL, aPe, nw, K, nk, nC, cyl, o); #c1(RBP4) c2(NP_006735) c3(retinol-binding protein 4 precursor) c4(167566, 167567, 167568) c5(167569, 167570, 167571, 167572, 167573, 167574, 167575, 167576, 167577, 167578, 167579, 167580, 167581, 167582, 167583, 167584, 167585, 167586, 167587, 167588, 167589, 167590) c6( ) c7(bL, T, dD, aZ, ea, nx, e, uG, al, aA, jx, if, aj, v, j, ci, eq, qN, zP, fK, J, ad, GK, cyJ, cyK, F, oW, h, jy, ae, af, kl, zQ, d, A, je, fc, Mz, dq, aq, aF, ahI, f, ctm, gk, BU, iM, aPt, c, cO, hk, dh, U, mg, vy, mf, aS, dy, ei, gT, B); #c1(RBPJ) c2(NP_005340) c3(recombining binding protein suppressor of hairless isoform 1) c4(155240, 155241, 155242, 155243, 155244) c5(155245, 155246, 155247, 155248, 155249, 155250, 155251, 155252, 155253, 155254, 155255, 155256, 155257, 155258, 155259, 155260) c6( ) c7(T, al, aA, eA, ad, dP, bv, akN, aT, h, bj, iY, ji, ae, NM, Us, wC, d, aW, ack, aF, eF, eE, cm, dr, ck, U, cyL, y); #c1(RBPJ) c2(NP_056958) c3(recombining binding protein suppressor of hairless isoform 2) c4(188212, 188213) c5(188214, 188215, 188216, 188217, 188218, 188219) c6( ) c7(T, al, aA, eA, ad, dP, bv, akN, aT, h, bj, iY, ji, ae, NM, Us, wC, d, aW, ack, aF, eF, eE, cm, dr, ck, U, cyL, y); #c1(RBPJ) c2(NP_976028) c3(recombining binding protein suppressor of hairless isoform 3) c4(262733) c5(262734, 262735, 262736, 262737, 262738, 262739, 262740, 262741, 262742) c6( ) c7(T, al, aA, eA, ad, dP, bv, akN, aT, h, bj, iY, ji, ae, NM, Us, wC, d, aW, ack, aF, eF, eE, cm, dr, ck, U, cyL, y); #c1(RBPJ) c2(NP_976029) c3(recombining binding protein suppressor of hairless isoform 4) c4(262743, 262744, 262745, 262746, 262747, 262748, 262749, 262750, 262751) c5(262752, 262753, 262754, 262755, 262756, 262757, 262758, 262759, 262760, 262761, 262762, 262763, 262764, 262765, 262766, 262767, 262768, 262769, 262770, 262771, 262772, 262773, 262774, 262775, 262776, 262777, 262778, 262779, 262780, 262781, 262782, 262783, 262784, 262785, 262786, 262787, 262788, 262789, 262790, 262791, 262792, 262793, 262794, 262795, 262796, 262797, 262798, 262799, 262800, 262801, 262802, 262803, 262804, 262805, 262806, 262807, 262808, 262809, 262810, 262811, 262812, 262813, 262814, 262815) c6( ) c7(T, al, aA, eA, ad, dP, bv, akN, aT, h, bj, iY, ji, ae, NM, Us, wC, d, aW, ack, aF, eF, eE, cm, dr, ck, U, cyL, y); #c1(RBPJ) c2(XP_006714026) c3(recombining binding protein suppressor of hairless isoform X2) c4( ) c5(287495, 287496, 287497, 287498, 287499, 287500, 287501, 287502, 287503, 287504, 287505, 287506, 287507, 287508, 287509) c6( ) c7(T, al, aA, eA, ad, dP, bv, akN, aT, h, bj, iY, ji, ae, NM, Us, wC, d, aW, ack, aF, eF, eE, cm, dr, ck, U, cyL, y); #c1(RBPMS2) c2(NP_919248) c3(RNA-binding protein with multiple splicing 2) c4(258293) c5(258294, 258295, 258296, 258297, 258298, 258299) c6( ) c7(fb, T, cyM); #c1(RBSN) c2(NP_001289307) c3 (rabenosyn-5) c4(114138, 114139, 114140) c5(114141, 114142, 114143, 114144, 114145, 114146, 114147, 114148, 114149, 114150, 114151, 114152, 114153, 114154, 114155, 114156, 114157, 114158, 114159, 114160, 114161, 114162, 114163) c6( ) c7(ad); #c1(RBSN) c2(XP_011532304) c3(rabenosyn-5 isoform X4) c4(328467) c5(328468, 328469, 328470, 328471, 328472) c6( ) c7(ad); #c1(RBX1) c2(NP_055063) c3(E3 ubiquitin-protein ligase RBX1) c4(178682) c5(178683, 178684, 178685, 178686, 178687, 178688) c6( ) c7(XX, T, aW, go, aq, dL, aS, fr, iR, eL, B); #c1(RC3H1) c2(NP_001287780) c3(roquin-1 isoform c) c4(112857) c5(112858, 112859, 112860, 112861, 112862, 112863) c6( ) c7(mC, fL, eZ); #c1(RC3H1) c2(NP_742068) c3(roquin-1 isoform b) c4(247957) c5(247958, 247959, 247960, 247961, 247962, 247963, 247964, 247965, 247966, 247967) c6( ) c7(mC, fL, eZ); #c1(RCAN1) c2(NP_001272318) c3(calcipressin-1 isoform d) c4( ) c5(103985, 103986, 103987) c6( ) c7(T, dD, ea, aw, bD, bhL, eq, nA, q, md, av, anj, hd, Hx, hb, aT, cy, ji, K, af, cY, d, Y, Az, r, Ku, g, f, hq, es, rR, ac, iA, nB, AM, wL, bbA, mx, tV, cj, aS, xh, y, ao); #c1(RCAN1) c2(NP_001272320) c3(calcipressin-1 isoform e) c4(103988) c5(103989, 103990, 103991, 103992, 103993, 103994) c6( ) c7(T, dD, ea, aw, bD, bhL, eq, nA, q, md, av, anj, hd, Hx, hb, aT, cy, ji, K, af, cY, d, Y, Az, r, Ku, g, f, hq, es, rR, ac, iA, nB, AM, wO, bbA, mx, tV, cj, aS, xh, y, ao); #c1(RCAN1) c2(NP_001272322) c3(calcipressin-1 isoform b) c4( ) c5(103995, 103996, 103997, 103998, 103999, 104000, 104001, 104002, 104003, 104004, 104005) c6( ) c7(T, dD, ea, aw, bD, bhL, eq, nA, q, md, av, anj, hd, Hx, hb, aT, cy, ji, K, af, cY, d, Y, Az, r, Ku, g, f, hq, es, rR, ac, iA, nB, AM, wO, bbA, mx, tV, cj, aS, xh, y, ao); #c1(RCAN1) c2(NP_004405) c3(calcipressin-I isoform a) c4(146147) c5(146148, 146149, 146150, 146151, 146152, 146153) c6( ) c7(T, dD, ea, aw, bD, bhL, eq, nA, q, md, av, anj, hd, Hx, hb, aT, cy, ji, K, af, cY, d, Y, Az, r, Ku, g, f, hq, es, rR, ac, iA, nB, AM, wL, bbA, mx, tV, cj, aS, xh, y, ao); #c1(RCAN1) c2(NP_981963) c3(calcipressin-1 isoform c) c4(263219, 263220) c5(263221, 263222, 263223, 263224, 263225, 263226) c6( ) c7(T, dD, ea, aw, bD, bhL, eq, nA, q, md, av, anj, hd, Hx, hb, aT, cy, ji, K, af, cY, d, Y, Az, r, Ku, g, f, hq, es, rR, ac, iA, nB, AM, wO, bbA, mx, tV, cj, aS, xh, y, ao); #c1(RCAN2) c2(NP_005813) c3(calcipressin-2 isoform 1) c4(159633) c5(159634, 159635, 159636, 159637, 159638, 159639) c6( ) c7(f, anj, HN); #c1(RCAN2) c2(XP_011512528) c3(calcipressin-2 isoform X1) c4(303170) c5(303171, 303172, 303173, 303174, 303175, 303176, 303177, 303178, 303179, 303180, 303181, 303182, 303183, 303184) c6( ) c7(f, anj, HN); #c1(RCBTB1) c2(XP_005266498) c3(RCC1 and BTB domain-containing protein 1 isoform X1) c4(280193, 280194) c5(280195, 280196, 280197, 280198, 280199, 280200, 280201, 280202, 280203, 280204, 280205, 280206, 280207, 280208, 280209, 280210) c6( ) c7(eF, bM, eH, mQ, cj); #c1(RCBTB2) c2(NP_001273759) c3(RCC1 and BTB domain-containing protein 2 isoform 1) c4(105788, 105789, 105790) c5(105791, 105792, 105793, 105794, 105795, 105796, 105797, 105798, 105799, 105800, 105801, 105802, 105803, 105804, 105805, 105806, 105807, 105808) c6( ) c7(zJ, ae, fd, ad, hj); #c1(RCBTB2) c2(NP_001273760) c3(RCC1 and BTB domain-containing protein 2 isoform 3) c4( ) c5(105809, 105810, 105811, 105812, 105813) c6( ) c7(zJ, ae, fd, ad, hj); #c1(RCBTB2) c2(NP_001273761) c3(RCC1 and BTB domain-containing protein 2 isoform 4) c4(105814) c5(105815, 105816, 105817, 105818, 105819, 105820) c6( ) c7(zJ, ae, fd, ad, hj); #c1(RCHY1) c2(NP_001009922) c3(RING finger and CHY zinc finger domain-containing protein 1 isoform 3) c4(23728) c5(23729, 23730, 23731, 23732, 23733, 23734) c6( ) c7(es, T, py, iA, aT, n, K, y, ik, ae, B, ad); #c1(RCHY1) c2(NP_056251) c3(RING finger and CHY zinc finger domain-containing protein 1 isoform 1) c4(186727, 186728, 186729) c5(186730, 186731, 186732, 186733, 186734, 186735, 186736, 186737, 186738, 186739, 186740, 186741, 186742, 186743, 186744, 186745, 186746, 186747) c6( ) c7(es, T, py, iA, aT, n, K, y, iK, ae, B, ad); #c1(RCL1) c2(NP_001273629) c3(RNA 3′-terminal phosphate cyclase-like protein isoform b) c4( ) c5(105500, 105501, 105502, 105503, 105504) c6( ) c7(agy, cd, awC, iV); #c1(RCL1) c2(NP_005763) c3(RNA 3′-terminal phosphate cyclase-like protein isoform a) c4(159146, 159147) c5(159148, 159149, 159150, 159151, 159152, 159153, 159154, 159155, 159156, 159157, 159158, 159159) c6( ) c7(agy, cd, awC, iV); #c1(RCL1) c2(XP_006716778) c3(RNA 3′-terminal phosphate cyclase-like protein isoform X2) c4(290449) c5(290450) c6( ) c7(agy, cd, awC, iV); #c1(RCL1) c2(XP_011515975) c3(RNA 3′-terminal phosphate cyclase-like protein isoform X1) c4(309128, 309129) c5(309130, 309131, 309132, 309133, 309134, 309135, 309136, 309137, 309138, 309139, 309140, 309141, 309142, 309143, 309144, 309145, 309146, 309147, 309148, 309149) c6( ) c7(agy, cd, awC, iV); #c1(RCN1) c2(NP_002892) c3(reticulocalbin-1 precursor) c4(133264, 133265) c5(133266, 133267, 133268, 133269, 133270, 133271, 133272, 133273, 133274, 133275, 133276, 133277, 133278, 133279) c6( ) c7(es, zQ, iA, aA, aF, ci, zP, ae, ad); #c1(RCN2) c2(NP_002893) c3(reticulocalbin-2 isoform a precursor) c4(133280) c5(133281, 133282, 133283, 133284, 133285, 133286) c6( ) c7(T, HN); #c1(RCOR1) c2(NP_055971) c3(REST corepressor 1) c4(184800) c5(184801, 184802, 184803, 184804, 184805, 184806, 184807, 184808, 184809, 184810, 184811, 184812) c6( ) c7(nf, ey, cdo, bz, aAe); #c1(RCSD1) c2(NP_443094) c3(capZ-interacting protein) c4(229363, 229364) c5(229365, 229366, 229367, 229368, 229369, 229370, 229371, 229372, 229373, 229374, 229375, 229376) c6( ) c7(k, bA, cz); #c1(RCSD1) c2(XP_005245662) c3(capZ-interacting protein isoform X1) c4(266869) c5(266870, 266871, 266872, 266873, 266874, 266875) c6( ) c7(k, bA, cz); #c1(RCVRN) c2(NP_002894) c3(recoverin) c4(133287) c5(133288, 133289, 133290, 133291, 133292, 133293, 133294) c6( ) c7(T, ft, lR, bP, cwx, Y, nw, K, Tp, nk, nC, oc, cyN); #c1(RD3) c2(NP_001158160) c3(protein RD3) c4(63218) c5(63219, 63220, 63221, 63222, 63223, 63224) c6( ) c7(aIF, bFr, lR, c, nw, de); #c1(RDH10) c2(NP_742034) c3(retinol dehydrogenase 10) c4(247886) c5(247887, 247888, 247889, 247890, 247891, 247892) c6( ) c7(s, eM, nx, Fx, ci, V, fq, B); #c1(RDH11) c2(NP_057110) c3(retinol dehydrogenase 11 isoform 1 precursor) c4(188966, 188967) c5(188968, 188969, 188970, 188971, 188972, 188973, 188974, 188975, 188976, 188977, 188978, 188979) c6( ) c7(ae, B, ad, jh); #c1(RDH12) c2(NP_689656) c3(retinol dehydrogenase 12 precursor) c4(243493) c5(243494, 243495, 243496, 243497, 243498, 243499, 243500, 243501, 243502, 243503, 243504, 243505, 243506, 243507) c6( ) c7(xX, lR, bnP, nx, aVv, cyO, nw, K, eQ, nC, cyP, Nh); #c1(RDH14) c2(NP_065956) c3(retinol dehydrogenase 14) c4(206478) c5(206479, 206480, 206481, 206482, 206483, 206484) c6( ) c7(T, q, r); #c1(RDH16) c2(NP_003699) c3(retinol dehydrogenase 16) c4(140250) c5(140251, 140252, 140253, 140254, 140255, 140256) c6( ) c7(hL); #c1(RDH5) c2(NP_002896) c3(11-cis retinol dehydrogenase precursor) c4(133384) c5(133385, 133386, 133387, 133388, 133389, 133390, 133391, 133392, 133393, 133394) c6( ) c7(bL, cZ, cap, b, Sr, dd, lR, hl, aVu, nk, nC, ayj, bF, W, bnP, Be, NO, aSW, nw, OL, oQ, i); #c1(RDH8) c2(NP_056540) c3(retinol dehydrogenase 8) c4(188015) c5(188016, 188017, 188018, 188019, 188020, 188021) c6( ) c7(nw, Be, de); #c1(RDM1) c2(NP_001030008) c3(RAD52 motif-containing protein 1 isoform 2) c4(30823, 30824) c5(30825, 30826, 30827, 30828, 30829, 30830, 30831, 30832, 30833, 30834, 30835, 30836) c6( ) c7(bX); #c1(RDM1) c2(NP_001156592) c3(RAD52 motif-containing protein 1 isoform 3) c4(61752) c5(61753, 61754, 61755, 61756, 61757, 61758) c6( ) c7(bX); #c1(RDM1) c2(NP_001156593) c3(RAD52 motif-containing protein 1 isoform 4) c4(61759, 61760) c5(61761, 61762, 61763, 61764, 61765, 61766) c6( ) c7(bX); #c1(RDM1) c2(NP_001156594) c3(RAD52 motif-containing protein 1 isoform 5) c4( ) c5(61767, 61768, 61769, 61770, 61771, 61772) c6( ) c7(bX); #c1(RDM1) c2(NP_001156596) c3(RAD52 motif-containing protein 1 isoform 6) c4(61773) c5(61774, 61775, 61776, 61777, 61778, 61779) c6( ) c7(bX); #c1(RDM1) c2(NP_001156597) c3(RAD52 motif-containing protein 1 isoform 7) c4(61780) c5(61781, 61782, 61783, 61784, 61785, 61786) c6( ) c7(bX); #c1(RDM1) c2(NP_001156602) c3(RAD52 motif-containing protein 1 isoform 8) c4(61787) c5(61788, 61789, 61790, 61791, 61792, 61793) c6( ) c7(bX); #c1(RDM1) c2(NP_663629) c3(RAD52 motif-containing protein 1 isoform 1) c4(241368) c5(241369, 241370, 241371, 241372, 241373, 241374) c6( ) c7(bX); #c1(RDM1) c2(XP_011522812) c3(RAD52 motif-containing protein 1 isoform X2) c4(317304) c5(317305, 317306, 317307, 317308, 317309, 317310) c6( ) c7(bX); #c1(RDX) c2(NP_001247421) c3(radixin isoform 1) c4(93526, 93527) c5(93528, 93529, 93530, 93531, 93532, 93533, 93534, 93535, 93536, 93537, 93538, 93539, 93540, 93541) c6( ) c7(T, aC, aw, aA, BF, eA, ad, ip, aT, Bh, ae, bc, iE, aW, cyQ, kK, hI, hC, aF, hJ, Ew, pZ, gn, ik, iu); #c1(RDX) c2(NP_001247423) c3(radixin isoform 3) c4(93542) c5(93543, 93544, 93545, 93546, 93547, 93548) c6( ) c7(T, aC, aw, aA, BF, eA, ad, ip, aT, Bh, ae, bc, iE, aW, cyQ, kK, hI, hC, aF, hJ, Ew, pZ, gn, ik, iu); #c1(RDX) c2(NP_001247424) c3(radixin isoform 4) c4(93549, 93550) c5(93551, 93552, 93553, 93554, 93555, 93556, 93557, 93558, 93559, 93560, 93561, 93562) c6( ) c7(T, aC, aw, aA, BF, eA, ad, ip, aT, Bh, ae, bc, iE, aW, cyQ, kK, hI, hC, aF, hJ, Ew, pZ, gn, ik, iu); #c1(RDX) c2(NP_001247425) c3(radixin isoform 5) c4(93563) c5(93564, 93565, 93566, 93567, 93568, 93569) c6( ) c7(T, aC, aw, aA, BF, eA, ad, ip, aT, Bh, ae, bc, iE, aW, cyQ, kK, hI, hC, aF, hJ, Ew, pZ, gn, ik, iu); #c1(RDX) c2(NP_002897) c3(radixin isoform 2) c4(133395) c5(133396, 133397, 133398, 133399, 133400, 133401, 133402, 133403, 133404, 133405, 133406, 133407) c6( ) c7(T, aC, aw, aA, BF, eA, ad, ip, aT, Bh, ae, bc, iE, aW, cyQ, kK, hI, hC, aF, hJ, Ew, pZ, gn, ik, iu); #c1(REC8) c2(NP_001041670) c3(meiotic recombination protein REC8 homolog) c4(34247, 34248, 34249) c5(34250, 34251, 34252, 34253, 34254, 34255, 34256, 34257, 34258, 34259, 34260, 34261, 34262, 34263, 34264, 34265, 34266, 34267, 34268, 34269, 34270, 34271) c6( ) c7(bD, Y, ad, ND, vH); #c1(RECK) c2(NP_066934) c3(reversion-inducing cysteine-rich protein with Kazal motifs precursor) c4(207580) c5(207581, 207582, 207583, 207584, 207585, 207586) c6( ) c7(bM, T, aC, aA, bB, n, fd, ci, qN, q, ad, eA, av, iu, jz, no, yt, aT, dL, hu, ae, eL, pI, Jj, eG, d, B, bf, aW, ff, eH, eZ, r, kp, aF, en, g, eF, t, cm, bz, c, eM, bE, gX, m, y, X, bH); #c1(RECQL) c2(XP_006719196) c3(ATP-dependent DNA helicase Q1 isoform X1) c4(292244, 292245, 292246, 292247, 292248, 292249, 292250) c5(292251, 292252, 292253, 292254, 292255, 292256, 292257, 292258, 292259, 292260, 292261, 292262, 292263, 292264, 292265, 292266, 292267, 292268, 292269, 292270, 292271, 292272, 292273, 292274, 292275, 292276, 292277, 292278, 292279, 292280, 292281, 292282, 292283, 292284, 292285, 292286, 292287, 292288, 292289, 292290, 292291, 292292, 292293, 292294, 292295, 292296) c6( ) c7(T, aC, axk, Gn, ci, eJ, aE, ey, K, ask, d, wr, hG, eH, aq, g, eF, t, iK, dr, m, rA); #c1(RECQL5) c2(NP_001003715) c3(ATP-dependent DNA helicase Q5 isoform 2) c4(20048) c5(20049, 20050, 20051, 20052, 20053, 20054) c6( ) c7(T, wr, aE, bE, QJ, eH, r, bB, Gn, K, eF, rA, q, dr); #c1(RECQL5) c2(NP_001003716) c3(ATP-dependent DNA helicase Q5 isoform 3) c4( ) c5(20055, 20056, 20057, 20058, 20059, 20060) c6( ) c7(T, wr, aE, bE, QJ, eH, r, bB, Gn, K, eF, rA, q, dr); #c1(RECQL5) c2(NP_004250) c3(ATP-dependent DNA helicase Q5 isoform 1) c4(144595, 144596) c5(144597, 144598, 144599, 144600, 144601, 144602, 144603, 144604, 144605, 144606, 144607, 144608) c6( ) c7(T, wr, aE, bE, QJ, eH, r, bB, Gn, K, eF, rA, q, dr); #c1(RECQL5) c2(XP_005257879) c3(ATP-dependent DNA helicase Q5 isoform X6) c4(275390) c5(275391, 275392, 275393, 275394, 275395, 275396) c6( ) c7(T, wr, aE, bE, QJ, eH, r, bB, Gn, K, eF, rA, q, dr); #c1(RECQL5) c2(XP_011523784) c3(ATP-dependent DNA helicase Q5 isoform X1) c4(318284) c5( ) c6( ) c7(T, wr, aE, bE, QJ, eH, r, bB, Gn, K, eF, rA, q, dr); #c1(REEP1) c2(NP_001158202) c3(receptor expression-enhancing protein 1 isoform 1) c4(63325) c5(63326, 63327, 63328, 63329, 63330, 63331) c6( ) c7(aV, cyS, cyR, dM, aN, U, ajb, vd, LK); #c1(REEP1) c2(NP_001158203) c3(receptor expression-enhancing protein 1 isoform 3) c4(63332, 63333) c5(63334, 63335, 63336, 63337, 63338, 63339, 63340, 63341, 63342, 63343, 63344, 63345) c6( ) c7(aV, cyS, cyR, dM, aN, U, ajb, vd, LK); #c1(REEP1) c2(NP_001158204) c3(receptor expression-enhancing protein 1 isoform 4) c4(63346) c5(63347, 63348, 63349, 63350, 63351, 63352) c6( ) c7(aV, cyS, cyR, dM, aN, U, ajb, vd, LK); #c1(REEP1) c2(NP_075063) c3(receptor expression-enhancing protein 1 isoform 2) c4(213632) c5(213633, 213634, 213635, 213636, 213637, 213638, 213639, 213640) c6( ) c7(aV, cyS, cyR, dM, aN, U, ajb, vd, LK); #c1(REEP2) c2(NP_001258732) c3(receptor expression-enhancing protein 2 isoform 1) c4(96436) c5(96437, 96438, 96439, 96440, 96441, 96442) c6( ) c7(aV, cyT); #c1(REEP2) c2(NP_057690) c3(receptor expression-enhancing protein 2 isoform 2) c4(192363) c5(192364, 192365, 192366, 192367, 192368, 192369) c6( ) c7(aV, cyT); #c1(REEP3) c2(NP_001001330) c3(receptor expression-enhancing protein 3) c4(18174, 18175, 18176) c5(18177, 18178, 18179, 18180, 18181, 18182, 18183, 18184, 18185, 18186, 18187, 18188, 18189, 18190, 18191, 18192, 18193, 18194) c6( ) c7(aw, bqS, bN); #c1(REEP5) c2(NP_005660) c3(receptor expression-enhancing protein 5) c4(158341, 158342) c5(158343, 158344, 158345, 158346, 158347, 158348, 158349, 158350, 158351, 158352, 158353) c6( ) c7(bF, av, T, B, ac, BO, af, e, X, ff, bO, r, jh, m, en, t, bz, q, L); #c1(REEP6) c2(NP_612402) c3(receptor expression-enhancing protein 6) c4(236392) c5(236393, 236394, 236395, 236396, 236397, 236398) c6( ) c7(bB, bE, T, fe, iV); #c1(REG1A) c2(NP_002900) c3(lithostathine-1-alpha precursor) c4(133416) c5(133417, 133418, 133419, 133420, 133421, 133422) c6( ) c7(T, yS, ci, bwZ, bJj, Ou, K, hH, eL, bc, YL, yo, JI, aW, aoo, LZ, NR, g, as, akf, eF, cm, bw, eM, Jr, iT, U, m, lS, act, qR, ht, fN, aj, fr, ft, kH, h, aKE, Lg, afu, A, fe, aq, kp, pm, ar, iO, bP, eD, FV, eo, agy, B, bL, aA, cz, v, yO, V, q, J, eA, bza, aT, j, aKG, ae, af, s, Y, r, lo, t, ck, iM, c, ao, Hn, dG, aNH, hF, ad, aFu, hX, av, NS, io, pq, je, bug, eH, cr, aF, hp, iV, bz, dr, aJx, es, hE, y); #c1(REG1B) c2(NP_006498) c3(lithostathine-1-beta precursor) c4(165735) c5(165736, 165737, 165738, 165739, 165740, 165741, 165742, 165743) c6(165744, 165745) c7(bL, A, fe); #c1(REG3A) c2(NP_620355) c3(regenerating islet-derived protein 3-alpha precursor) c4(237509) c5(237510, 237511, 237512, 237513, 237514, 237515, 237516, 237517, 237518, 237519, 237520, 237521, 237522, 237523, 237524, 237525, 237526, 237527, 237528, 237529) c6(237530, 237531, 237532, 237533, 237534, 237535) c7(bL, T, dA, yk, if, iW, bB, aj, v, iG, ci, qN, aY, cyU, cd, q, J, b, aE, i, aoA, bK, K, wb, af, yo, xX, d, s, Y, cr, r, aq, uF, iR, GR, ck, ar, iO, bE, wq, dT, zG, zI, B, ao); #c1(REL) c2(NP_001278675) c3(proto-oncogene c-Rel isoform 2) c4(109970) c5(109971, 109972, 109973, 109974, 109975, 109976) c6( ) c7(T, fM, iW, eX, jh, fd, ci, ai, wD, De, ot, aE, azq, aT, eV, dz, pq, aoX, aW, cr, eZ, lo, yq, hp, hf, eE, iV, iK, Jr, cj, aGg, y); #c1(REL) c2(NP_002899) c3(proto-oncogene c-Rel isoform 1) c4(133408) c5(133409, 133410, 133411, 133412, 133413, 133414, 133415) c6( ) c7(T, fM, iW, ex, jh, fd, ci, ai, wD, De, ot, aE, azq, aT, eV, dz, pq, aoX, aW, cr, eZ, lo, yq, hp, hf, eE, iV, ik, Jr, cj, aGg, y); #c1(RELA) c2(NP_001138610) c3(transcription factor p65 isoform 2) c4(57176) c5(57177, 57178, 57179, 57180, 57181, 57182) c6( ) c7(oO, bM, T, os, aC, fR, ea, if, al, bEy, eb, fd, lR, ci, bv, aE, Bc, K, xa, eG, aW, az, asD, LZ, fc, g, hJ, eE, eF, cm, bw, axG, nB, cj, m, hh, ht, aj, BF, fr, qN, qW, eW, kH, cU, lG, pi, wC, EU, gi, fe, aq, kp, cv, dB, hk, cB, B, it, bL, fM, eR, aA, cz, C, V, q, J, eA, eJ, uT, yg, aT, bq, ae, af, eu, aAi, jf, nO, Y, r, en, bu, t, f, iK, pb, ck, ov, iM, ac, c, ig, hr, EY, bt, qb, oA, oZ, dG, ah, hF, ad, XB, KL, dP, av, iC, hd, ib, yt, ds, azq, ji, vP, azR, gj, d, je, ff, xs, eZ, aF, iV, iR, DF, bF, aGg, lC, kS, X, y, brt); #c1(RELA) c2(NP_001230914) c3(transcription factor p65 isoform 4) c4( ) c5(87155, 87156, 87157, 87158, 87159, 87160, 87161, 87162, 87163, 87164, 87165, 87166, 87167, 87168, 87169, 87170, 87171, 87172, 87173, 87174, 87175, 87176) c6( ) c7(oO, bM, T, os, aC, fR, ea, if, al, bEy, eb, fd, lR, ci, bv, aE, Bc, K, xa, eG, aW, az, asD, LZ, fc, g, hJ, BE, eF, cm, bw, axG, nB, cj, m, hh, ht, aj, BF, fr, qN, qW, eW, kH, cU, lG, pi, wC, EU, gi, fe, aq, kp, cv, dB, hk, cB, B, it, bL, fM, eR, aA, cz, C, V, q, J, eA, eJ, uT, yg, aT, bq, ae, af, eu, aAi, jf, nO, Y, r, en, bu, t, f, ik, pb, ck, ov, iM, ac, c, ig, hr, EY, bt, qb, oA, oZ, dG, ah, hF, ad, XB, kL, dP, av, iC, hd, ib, yt, ds, azq, ji, vP, azR, gj, d, je, ff, xs, eZ, aF, iV, iR, DF, bF, aGg, lC, KS, X, y, brt); #c1(RELA) c2(NP_068810) c3 (transcription factor p65 isoform 1) c4(210689, 210690, 210691, 210692, 210693) c5(210694, 210695, 210696, 210697, 210698, 210699) c6( ) c7(oO, bM, T, os, aC, fR, ea, if, al, bEy, eb, fd, lR, ci, bv, aE, Bc, K, xa, eG, aW, az, asD, LZ, fc, g, hJ, eE, eF, cm, bw, axG, nB, cj, m, hh, ht, aj, BF, fr, qN, qW, eW, kH, cU, lG, pi, wC, EU, gi, fe, aq, kp, cv, dB, hk, cB, B, it, bL, fM, eR, aA, cz, C, V, q, J, eA, eJ, uT, yg, aT, bq, ae, af, eu, aAi, jf, nO, Y, r, en, bu, t, f, iK, pb, ck, ov, iM, ac, c, ig, hr, EY, bt, qb, oA, oZ, dG, ah, hF, ad, XB, kL, dP, av, iC, hd, ib, yt, ds, azq, ji, vP, azR, gj, d, je, ff, xs, eZ, aF, iV, iR, DF, bF, aGg, lC, KS, X, y, brt); #c1 (RELA) c2(XP_011543508) c3(transcription factor p65 isoform X1) c4( ) c5(342383) c6( ) c7(oO, bM, T, os, aC, fR, ea, if, al, bEy, eb, fd, IR, ci, bv, aE, Bc, K, xa, eG, aW, az, asD, LZ, fc, g, hJ, eE, eF, cm, bw, axG, nB, cj, m, hh, ht, aj, BF, fr, qN, qW, eW, kH, cU, lG, pi, wC, EU, gi, fe, aq, kp, cv, dB, hk, cB, B, it, bL, fM, eR, aA, cz, C, V, q, J, eA, eJ, uT, yg, aT, bq, ae, af, eu, aAi, jf, nO, Y, r, en, bu, t, f, iK, pb, ck, ov, iM, ac, c, ig, hr, EY, bt, qb, oA, oZ, dG, ah, hF, ad, XB, kL, dP, av, iC, hd, ib, yt, ds, azq, ji, vP, azR, gj, d, je, ff, xs, eZ, aF, iV, iR, DF, bF, aGg, lC, kS, X, y, brt); #c1(RELA) c2(XP_011543509) c3(transcription factor p65 isoform X2) c4( ) c5(342384) c6( ) c7(oO, bM, T, os, aC, fR, ea, if, al, bEy, eb, fd, lR, ci, bv, aE, Bc, K, xa, eG, aW, az, asD, LZ, fc, g, hJ, eE, eF, cm, bw, axG, nB, cj, m, hh, ht, aj, BF, fr, qN, qW, eW, kH, cU, lG, pi, wC, EU, gi, fe, aq, kp, cv, dB, hk, cB, B, it, bL, fM, eR, aA, cz, C, V, q, J, eA, eJ, uT, yg, aT, bq, ae, af, eu, aAi, jf, nO, Y, r, en, bu, t, f, iK, pb, ck, ov, iM, ac, c, ig, hr, EY, bt, qb, oA, oZ, dG, ah, hF, ad, XB, kL, dP, av, iC, hd, ib, yt, ds, azq, ji, vP, azR, gj, d, je, ff, xs, eZ, aF, iV, iR, DF, bF, aGg, lC, kS, X, y, brt); #c1(RELB) c2(NP_006500) c3(transcription factor RelB) c4(165746) c5(165747, 165748, 165749, 165750, 165751, 165752, 165753, 165754, 165755, 165756) c6( ) c7(y, A, aW, eE, cj, aT, Mz, fd, dy, K, aGg, C, j, eA, bl, ae, B, ad, cl); #c1(RELB) c2(XP_005259184) c3(transcription factor RelB isoform X1) c4(275967) c5(275968, 275969, 275970, 275971, 275972, 275973) c6( ) c7(y, A, aW, eE, cj, aT, Mz, fd, dy, K, aGg, C, j, eA, bl, ae, B, ad, cl); #c1(RELN) c2(NP_005036) c3(reelin isoform a precursor) c4(152326, 152327, 152328, 152329) c5(152330, 152331, 152332, 152333, 152334, 152335, 152336, 152337, 152338, 152339, 152340, 152341, 152342, 152343, 152344, 152345, 152346, 152347, 152348, 152349, 152350, 152351, 152352, 152353) c6( ) c7(nD, aC, oe, ha, aA, n, ct, il, qi, V, cyV, aE, hb, ds, aT, bK, K, hu, he, H, rz, af, Lg, cB, VX, aW, FD, hl, fQ, gI, aF, cm, bN, cA, bE, FB, gT, xg, B, L); #c1(RELN) c2(NP_774959) c3(reelin isoform b precursor) c4(249481) c5(249482, 249483, 249484, 249485, 249486, 249487) c6( ) c7(nD, aC, oe, ha, aA, n, ct, il, qi, V, cyV, aE, hb, ds, aT, bK, K, hu, he, H, rz, af, Lg, cB, VX, aW, FD, hl, fQ, gI, aF, cm, bN, cA, bE, FB, gT, xg, B, L); #c1(REM1) c2(NP_054731) c3(GTP-binding protein REM 1) c4(177416) c5(177417, 177418, 177419, 177420, 177421, 177422) c6( ) c7(T, cyW, bO, Nt, aA, aj, bS, j, ct, v, bAM, Eh, J, cF, wM, M, bHp, K, he, alY, alL, az, aF, Z, hq, dR, ayt, cA, vy, uJ, F1); #c1(REN) c2(NP_000528) c3(renin preproprotein) c4(11894, 11895, 11896) c5(11897, 11898, 11899, 11900, 11901, 11902, 11903, 11904, 11905, 11906, 11907, 11908, 11909, 11910, 11911, (1912, 11913, 11914) c6( ) c7(tJ, T, UH, ahS, Fj, fV, ea, e, al, Xi, eb, MF, HS, aoM, aY, x, abH, ws, il, M, Mx, iv, Ou, K, Nj, aDZ, dQ, wt, eN, bc, hm, tw, avH, aW, Fe, hl, bbe, bWB, zC, uC, DL, GR, cyZ, mx, U, vy, wB, k, Hb, xh, fy, amz, dD, uH, bLI, wJ, aw, cd, aj, fr, ct, ml, cd, aJr, bSq, ft, cc, do, ec, wG, ql, cty, h, jy, afV, ey, bl, he, vx, bkS, A, pm, dw, wU, sf, hq, kT, aLO, bbj, vb, FV, bo, bm, MM, tl, uz, oh, va, hR, fL, at, bD, sZ, aOo, um, bL, vQ, jv, eR, mr, DD, dk, aA, cR, tc, cz, fF, j, v, wo, sl, il, uW, q, J, er, Mp, XD, tL, rS, aT, cy, bq, hK, Fg, ae, aqC, af, dL, bA, eu, jf, Mr, s, nO, r, wL, sV, lo, An, Mu, tR, sk, ck, tY, tW, ac, wg, hc, kQ, RW, fP, mf, cS, cIP, dy, bt, LS, hS, gx, sY, dM, ha, uG, Jy, vr, bS, Jz, uu, dG, MA, eq, tK, mC, ad, fU, nH, F, MS, cyY, ib, sN, vJ, arV, ts, oW, vw, cyX, vc, vP, sj, uX, anR, d, cH, cW, xs, cr, eZ, dq, th, aF, si, Z, dr, qa, ek, pc, ag, gZ, aS, tr, ei, lC, us); #c1(RENBP) c2(NP_002901) c3(N-acylglucosamine 2-epimerase) c4(133423, 133424) c5(133425, 133426, 133427, 133428, 133429, 133430, 133431, 133432, 133433, 133434, 133435, 133436) c6( ) c7(bL, T, dD, ea, if, aw, eX, aA, cz, oR, aj, bS, dG, j, v, eq, qN, aY, J, alp, Ot, qh, oW, Ak, eV, dL, wo, K, dQ, H, eL, uX, A, d, bf, je, bvD, eZ, aq, aF, as, bSy, aIj, qa, aIm, BU, W, c, ac, rR, iM, mx, U, fP, vy, MW, aS, dy, yA, Lz, B); #c1 (REPS1) c2(NP_001122089) c3(ralBP1-associated Eps domain-containing protein 1 isoform b) c4(46434) c5(46435, 46436, 46437, 46438, 46439, 46440) c6( ) c7(ck, ae, T, eG, aE, eM, eL, ha, aT, dL, K, bz, ad); #c1(REPS1) c2(NP_001273540) c3(ralBP1-associated Eps domain-containing protein 1 isoform c) c4(105131, 105132) c5(105133, 105134, 105135, 105136, 105137, 105138, 105139, 105140, 105141, 105142, 105143, 105144) c6( ) c7(ck, ae, T, eG, aE, eM, eL, ha, aT, dL, K, bz, ad); #c1(REPS1) c2(NP_001273541) c3(ralBP1-associated Eps domain-containing protein 1 isoform d) c4(105145, 105146) c5(105147, 105148, 105149, 105150, 105151, 105152) c6( ) c7(ck, ae, T, eG, aE, eM, eL, ha, aT, dL, K, bz, ad); #c1(REPS1) c2(NP_114128) c3(ralBP1-associated Eps domain-containing protein 1 isoform a) c4(222278) c5(222279, 222280, 222281, 222282, 222283, 222284) c6( ) c7(ck, ae, T, eG, aE, eM, eL, ha, aT, dL, K, bz, ad); #c1(REPS1) c2(XP_011534505) c3(ralBP1-associated Eps domain-containing protein 1 isoform X6) c4( ) c5(330968, 330969, 330970, 330971, 330972, 330973) c6( ) c7(ck, ae, T, eG, aE, eM, eL, ha, aT, dL, K, bz, ad); #c1(REPS2) c2(NP_001074444) c3(ralBP1-associated Eps domain-containing protein 2 isoform 2) c4(37113) c5(37114, 37115, 37116, 37117, 37118, 37119) c6( ) c7(eM, aW, aT, iJ, ae, ad); #c1(REPS2) c2(NP_004717) c3(ralBP1-associated Eps domain-containing protein 2 isoform 1) c4(149050, 149051) c5(149052, 149053, 149054, 149055, 149056, 149057) c6( ) c7(eM, aW, al, iJ, ae, ad); #c1(REPS2) c2(XP_005274683) c3(ralBP1-associated Eps domain-containing protein 2 isoform X7) c4(285330) c5(285331, 285332, 285333, 285334, 285335, 285336) c6( ) c7(eM, aW, aT, iJ, ae, ad); #c1(REPS2) c2(XP_006724586) c3(ralBP1-associated Eps domain-containing protein 2 isoform X4) c4( ) c5(296102, 296103, 296104, 296105, 296106, 296107) c6( ) c7(eM, aW, aT, iJ, ae, ad); #c1(RERG) c2(NP_001177655) c3(ras-related and estrogen-regulated growth inhibitor isoform 2) c4(72830) c5(72831, 72832, 72833, 72834, 72835, 72836) c6( ) c7(T, aW, aT, at, dL, ci); #c1(RERG) c2(NP_116307) c3(ras-related and estrogen-regulated growth inhibitor isoform 1) c4(226105, 226106) c5(226107, 226108, 226109, 226110, 226111, 226112, 226113, 226114, 226115, 226116, 226117, 226118, 226119, 226120, 226121, 226122, 226123, 226124, 226125, 226126) c6( ) c7(T, aW, aT, at, dL, ci); #c1(RERGL) c2(NP_079006) c3(ras-related and estrogen-regulated growth inhibitor-like protein isoform a) c4(216531) c5(216532, 216533, 216534, 216535, 216536, 216537) c6( ) c7(aW, q, aT, r, pN); #c1(REST) c2(NP_001180437) c3(REI-silencing transcription factor) c4(74052, 74053, 74054) c5(74055, 74056, 74057, 74058, 74059, 74060, 74061, 74062, 74063, 74064, 74065, 74066, 74067, 74068, 74069, 74070, 74071, 74072, 74073) c6( ) c7(T, ha, ex, bO, Hn, n, ci, Fc, eA, ad, ft, M, aT, cy, eV, K, ae, dx, eu, KC, jf, d, aW, Y, nf, bSc, t, f, bz, cm, ek, rR, W, auo, ac, dB, mx, U, gZ, m, xg); #c1(RET) c2(NP_065681) c3(proto-oncogene tyrosine-protein kinase receptor Ret isoform c precursor) c4(204815, 204816) c5(204817, 204818, 204819, 204820, 204821, 204822, 204823, 204824, 204825, 204826, 204827, 204828) c6( ) c7(uB, gh, atT, T, aCh, aC, Tb, cze, bB, ci, bjN, aon, xY, aR, bjV, Vp, XW, xy, aE, pX, cyn, K, pk, azh, li, yo, aCg, bn, EV, aW, Ls, Qv, ahF, Qq, dg, g, cm, QA, aAW, qJ, eM, nB, Ji, iS, bjU, QF, azg, iu, dD, wu, sw, n, ml, atV, SW, cza, aJe, aCi, aJf, ft, Do, azj, wG, KN, eV, dL, btr, aMi, Hu, aay, cM, bzv, Sc, YE, W, dB, hk, hR, at, pg, HB, qF, YH, anx, cB, AN, aAf, dA, AS, eR, ex, fZ, aA, cz, hy, hz, bX, q, er, eJ, Kx, mj, cng, czb, nv, Qg, aT, dz, Fx, an, ahE, eu, jf, Sh, wr, s, nO, iU, Y, r, ahl, czd, hf, t, f, Vi, FT, ck, sS, tY, Dy, c, rR, auz, nM, dy, gT, bt, VB, ahW, pY, Oy, Dd, uO, oH, eq, ad, av, hd, Rq, Db, bjT, wm, yh, Yi, aAL, BN, aus, et, eZ, bfu, czc, aF, aTa, HI, bz, dr, aHY, Ew, bE, Lj, Km, ne, jr, DB); #c1(RET) c2(NP_066124) c3(proto-oncogene tyrosine-protein kinase receptor Ret isoform a precursor) c4(206803) c5(206804, 206805, 206806, 206807, 206808, 206809) c6( ) c7(uB, gh, atT, T, aCh, aC, Tb, cze, bB, ci, bjN, aon, xY, aR, bjV, Vp, XW, xy, aE, pX, cyn, K, pk, azh, li, yo, aCg, bn, EV, aW, Ls, Qv, ahF, Qq, dg, g, cm, QA, aAW, qJ, eM, nB, Ji, iS, bjU, QF, azg, iu, dD, wu, sw, n, ml, atV, SW, cza, aJe, aCi, aJf, ft, Do, azj, wG, KN, eV, dL, btr, aMi, Hu, aay, cM, bzv, Sc, YE, W, dB, hk, hR, at, pg, HB, qF, YH, anx, CB, AN, aAf, dA, AS, eR, eX, fZ, aA, cz, hy, hz, bX, q, er, eJ, Kx, mj, cng, czb, nv, Qg, aT, dz, Fx, an, ahE, eu, jf, Sh, wr, s, nO, iU, Y, r, ahl, czd, hf, t, f, Vi, FT, ck, sS, tY, Dy, c, rR, auz, nM, dy, gT, bt, VB, ahW, pY, Oy, Dd, uO, oH, eq, ad, av, hd, Rq, Db, bjT, wm, yh, Yi, aAL, BN, aus, et, eZ, bfu, czc, aF, aTa, HI, bz, dr, aHY, Ew, bE, Lj, Km, ne, jr, DB); #c1(RETN) c2(NP_001180303) c3(resistin precursor) c4(73769, 73770) c5(73771, 73772, 73773, 73774, 73775, 73776, 73777, 73778, 73779, 73780, 73781, 73782) c6( ) c7(bL, bM, T, dD, eR, fR, aZ, ea, oe, e, uG, al, cz, cR, fN, aj, dG, j, v, eq, bh, nS, q, J, cd, ai, td, fU, rc, aw, cc, Qg, iX, ts, aT, h, jy, vc, GT, ji, K, uV, qM, nJ, uX, kl, bf, cW, aW, fc, hl, eZ, r, dq, bz, GR, bJ, ac, X, hr, bm, gZ, GN, aS, dy, mf, ei, gT, jd, st, y, vQ); #c1(RETNLB) c2(NP_115968) c3(resistin-like beta precursor) c4(224974) c5(224975, 224976, 224977, 224978, 224979, 224980, 224981, 224982) c6(224983, 224984) c7(LB, Kd, ck, dD, bM, ea, fG, Ox, eq, ji, fD, X, aa); #c1(RETSAT) c2(NP_060220) c3(all-trans-retinol 13,14-reductase precursor) c4(194819, 194820, 194821) c5(194822, 194823, 194824, 194825, 194826, 194827, 194828, 194829, 194830, 194831, 194832, 194833, 194834, 194835, 194836, 194837, 194838, 194839) c6( ) c7(fg); #c1(REV1) c2(NP_001032961) c3(DNA repair protein REV1 isoform 2) c4(31503) c5(31504, 31505, 31506, 31507, 31508, 31509) c6( ) c7(gV, T, os, hd, nB, hG, Wz, if, ff, asD, cn, aT, cj, oZ, K, ci, m, en, qN, bz, ip); #c1(REV1) c2(NP_057400) c3(DNA repair protein REV1 isoform 1) c4(190553) c5(190554, 190555, 190556, 190557, 190558, 190559) c6( ) c7(gV, T, os, hd, nB, hG, Wz, if, ff, asD, cn, aT, cj, oZ, K, ci, m, en, qN, bz, ip); #c1(REV3L) c2(NP_001273361) c3(DNA polymerase zeta catalytic subunit isoform b) c4(104762) c5(104763, 104764, 104765, 104766, 104767) c6( ) c7(T, fM, ib, bE, aW, cr, aT, cj, dL, ci, K, brf, bz, eA, hS); #c1(REV3L) c2(NP_002903) c3(DNA polymerase zeta catalytic subunit isoform a) c4(133444) c5(133445, 133446, 133447, 133448, 133449, 133450, 133451, 133452, 133453, 133454, 133455, 133456, 133457, 133458, 133459) c6( ) c7(T, fM, ib, bE, aW, cr, aT, cj, dL, ci, K, brf, bz, eA, hS); #c1(REV3L) c2(XP_011534337) c3(DNA polymerase zeta catalytic subunit isoform X9) c4(330713) c5(330714, 330715, 330716, 330717, 330718, 330719) c6( ) c7(T, fM, ib, bE, aW, cr, aT, cj, dL, ci, K, brf, bz, eA, hS); #c1(REXD1) c2(NP_065746) c3(RNA exonuclease 1 homolog) c4(205161, 205162) c5(205163, 205164, 205165, 205166, 205167, 205168, 205169, 205170, 205171, 205172, 205173, 205174, 205175) c6( ) c7(es, T, kl, d, Uc, iM, Db, je, LX, Hn, lu, ae, eA, ad); #c1(REXD1) c2(XP_011526450) c3(RNA exonuclease 1 homolog isoform X5) c4(320754) c5(320755, 320756, 320757, 320758, 320759) c6( ) c7(es, T, kl, d, Uc, iM, Db, je, LX, Hn, lu, ae, eA, ad); #c1(REXD2) c2(NP_056338) c3(oligoribonuclease, mitochondrial precursor) c4(187151) c5(187152, 187153, 187154, 187155, 187156, 187157) c6( ) c7(T, bE, aW, sw, aT, fd, n, g, zL, bz, iu); #c1(REXD4) c2(NP_001266278) c3(RNA exonuclease 4 isoform 2) c4(99554, 99555) c5(99556, 99557, 99558, 99559, 99560, 99561, 99562, 99563, 99564, 99565, 99566, 99567) c6( ) c7(bD, ac); #c1(REXD4) c2(NP_001266280) c3(RNA exonuclease 4 isoform 4) c4( ) c5(99568, 99569, 99570, 99571, 99572) c6( ) c7(bD, ac); #c1(REXD4) c2(NP_065118) c3(RNA exonuclease 4 isoform 1) c4(204023, 204024, 204025) c5(204026, 204027, 204028, 204029, 204030, 204031, 204032, 204033, 204034, 204035, 204036, 204037, 204038) c6( ) c7(bD, ac); #c1(RFC1) c2(NP_001191676) c3(replication factor C subunit 1 isoform 2) c4(81606) c5(81607, 81608, 81609, 81610, 81611, 81612) c6( ) c7(ng, T, if, jh, aA, sM, cz, oY, ci, V, q, ad, ip, wD, x, yg, aT, dL, K, eL, af, iL, Uf, aoX, aW, ff, eH, r, aF, en, g, eF, f, ik, y, ac, eM, bP, dB, lp, wE, dy, k, ij, bD, Qz, X, us); #c1(RFC1) c2(NP_002904) c3(replication factor C subunit 1 isoform 1) c4(133460) c5(133461, 133462, 133463, 133464, 133465, 133466) c6( ) c7(ng, T, if, jh, aA, sM, cz, oY, ci, V, q, ad, ip, wD, x, yg, aT, dL, K, eL, af, iL, Uf, aoX, aW, ff, eH, r, af, en, g, eF, f, ik, y, ac, eM, bP, dB, lp, wE, dy, k, ij, bD, Qz, X, us); #c1(RFC1) c2(XP_011512033) c3(replication factor C subunit 1 isoform X2) c4(302686) c5(302687, 302688, 302689, 302690, 302691, 302692) c6( ) c7(ng, T, if, jh, aA, sM, cz, oY, ci, V, q, ad, ip, wD, x, yg, aT, dL, K, eL, af, iL, Uf, aoX, aW, ff, eH, r, aF, en, g, eF, f, ik, y, ac, eM, bP, dB, lp, wE, dy, k, ij, bD, Qz, X, us); #c1(RFC2) c2(NP_002905) c3(replication factor C subunit 2 isoform 2) c4(133467) c5(133468, 133469, 133470, 133471, 133472, 133473) c6( ) c7(bL, bM, aIC, W, aW, eH, aT, Mz, dL, v, ei, eF, cm, V); #c1(RFC2) c2(NP_852136) c3(replication factor C subunit 2 isoform 1) c4(254908) c5(254909, 254910, 254911, 254912, 254913, 254914, 254915, 254916) c6( ) c7(bL, bM, aIC, W, aW, eH, aT, Mz, dL, v, ei, eF, cm, V); #c1(RFC3) c2(NP_002906) c3(replication factor C subunit 3 isoform 1) c4(133474) c5(133475, 133476, 133477, 133478, 133479, 133480) c6( ) c7(T, ac, pf, r, dL, np, j, af); #c1(RFC3) c2(NP_853536) c3(replication factor C subunit 3 isoform 2) c4(255171) c5(255172, 255173, 255174, 255175, 255176, 255177) c6( ) c7(T, ac, pf, r, dL, np, j, af); #c1(RFC4) c2(NP_853551) c3(replication factor C subunit 4) c4(255178, 255179, 255180, 255181, 255182, 255183) c5(255184, 255185, 255186, 255187, 255188, 255189, 255190, 255191, 255192, 255193, 255194, 255195, 255196, 255197, 255198, 255199, 255200, 255201, 255202, 255203, 255204, 255205, 255206, 255207, 255208, 255209, 255210, 255211, 255212, 255213, 255214, 255215, 255216, 255217, 255218, 255219, 255220, 255221, 255222, 255223) c6( ) c7(V, d, B, dL); #c1(RFC5) c2(NP_001123584) c3(replication factor C subunit 5 isoform 3) c4(47788) c5(47789, 47790, 47791, 47792, 47793) c6( ) c7(V, dL); #c1(RFC5) c2(NP_031396) c3(replication factor C subunit 5 isoform 1) c4(172223) c5(172224, 172225, 172226, 172227, 172228, 172229, 172230) c6( ) c7(V, dL); #c1(RFC5) c2(NP_853556) c3(replication factor C subunit 5 isoform 2) c4(255224, 255225, 255226) c5(255227, 255228, 255229, 255230, 255231, 255232, 255233, 255234, 255235, 255236, 255237, 255238, 255239, 255240, 255241, 255242, 255243, 255244) c6( ) c7(V, dL); #c1(RFFL) c2(NP_001017368) c3(E3 ubiquitin-protein ligase rififylin) c4(26288, 26289, 26290, 26291, 26292, 26293, 26294) c5(26295, 26296, 26297, 26298, 26299, 26300, 26301, 26302, 26303, 26304, 26305, 26306, 26307, 26308, 26309, 26310, 26311, 26312, 26313, 26314, 26315, 26316, 26317, 26318, 26319, 26320, 26321, 26322, 26323, 26324, 26325, 26326, 26327, 26328, 26329, 26330, 26331, 26332, 26333, 26334, 26335, 26336, 26337, 26338, 26339, 26340) c6( ) c7(cF); #c1(RFK) c2(NP_060809) c3(riboflavin kinase) c4(197931) c5(197932, 197933, 197934, 197935, 197936, 197937) c6( ) c7(ad); #c1(RFPL1) c2(NP_066306) c3(ret finger protein-like 1) c4(207169) c5(207170, 207171, 207172, 207173, 207174, 207175) c6( ) c7(dY, aRk); #c1(RFT1) c2(NP_443091) c3(protein RFT1 homolog) c4(229349, 229350) c5(229351, 229352, 229353, 229354, 229355, 229356, 229357, 229358, 229359, 229360, 229361, 229362) c6( ) c7(czf, Km, J, dv, aj); #c1(RFTN1) c2(NP_055965) c3(raftlin) c4(184728, 184729, 184730, 184731) c5(184732, 184733, 184734, 184735, 184736, 184737, 184738, 184739, 184740, 184741, 184742, 184743, 184744, 184745, 184746, 184747, 184748, 184749, 184750, 184751, 184752, 184753, 184754, 184755, 184756, 184757, 184758, 184759) c6( ) c7(hW, bE, vA, bB, v, dw, op); #c1(RFTN2) c2(NP_653230) c3(raftlin-2) c4(239241) c5(239242, 239243, 239244, 239245, 239246, 239247) c6( ) c7(Ea); #c1(RFWD2) c2(NP_001001740) c3(E3 ubiquitin-protein ligase RFWD2 isoform b) c4(19013) c5(19014, 19015, 19016, 19017, 19018, 19019) c6( ) c7(T, cF, aE, c, qt, aW, jx, aT, aA, n, aF, K, g, ir, bt, B, bN); #c1(RFWD2) c2(NP_001273573) c3(E3 ubiquitin-protein ligase RFWD2 isoform c precursor) c4(105323) c5( ) c6( ) c7(T, cF, aE, c, qt, aW, jx, aT, aA, n, aF, K, g, ir, bt, B, bN); #c1(RFWD2) c2(NP_071902) c3(E3 ubiquitin-protein ligase RFWD2 isoform a) c4(212163) c5(212164, 212165, 212166, 212167, 212168, 212169, 212170) c6( ) c7(T, cF, aE, c, qt, aW, jx, aT, aA, n, aF, K, g, ir, bt, B, bN); #c1(RFWD2) c2(XP_005245504) c3(E3 ubiquitin-protein ligase RFWD2 isoform X4) c4( ) c5(266665, 266666, 266667, 266668, 266669) c6( ) c7(T, cF, aE, c, qt, aW, jx, aT, aA, n, aF, K, g, ir, bt, B, bN); #c1(RFWD2) c2(XP_006711550) c3(E3 ubiquitin-protein ligase RFWD2 isoform X7) c4( ) c5(286119, 286120, 286121, 286122, 286123, 286124) c6( ) c7(T, cF, aE, c, qt, aW, jx, aT, aA, n, aF, K, g, ir, bt, B, bN); #c1(RFWD2) c2(XP_011508183) c3(E3 ubiquitin-protein ligase RFWD2 isoform X1) c4(298781) c5( ) c6( ) c7(T, cF, aE, c, qt, aW, jx, aT, aA, n, aF, K, g, ir, bt, B, bN); #c1(RFWD3) c2(NP_060594) c3(E3 ubiquitin-protein ligase RFWD3) c4(196733) c5(196734, 196735, 196736, 196737, 196738, 196739) c6( ) c7(Ld, ey); #c1(RFWD3) c2(XP_006721291) c3(E3 ubiquitin-protein ligase RFWD3 isoform X2) c4(293918) c5(293919, 293920, 293921, 293922, 293923, 293924, 293925) c6( ) c7(Ld, ey); #c1(RFX1) c2(NP_002909) c3(MHC class II regulatory factor RFX1) c4(133481) c5(133482, 133483, 133484, 133485, 133486, 133487, 133488) c6( ) c7(jf, T, B, d, dA, Pf, aW, eZ, aT, eo, fL, Tp, g, eA, Xa, V, WX, mC, MG); #c1(RFX1) c2(XP_011526469) c3(MHC class II regulatory factor RFX1 isoform X1) c4( ) c5(320775) c6( ) c7(jf, T, B, d, dA, Pf, aW, eZ, aT, eo, fL, Tp, g, eA, Xa, V, WX, mC, MG); #c1(RFX2) c2(NP_000626) c3(DNA-binding protein RFX2 isoform a) c4(14068) c5(14069, 14070, 14071, 14072, 14073, 14074, 14075) c6( ) c7(ayR); #c1(RFX2) c2(NP_602309) c3(DNA-binding protein RFX2 isoform b) c4(235764) c5(235765, 235766, 235767, 235768, 235769, 235770, 235771, 235772, 235773, 235774) c6( ) c7(ayR); #c1(RFX3) c2(NP_001269046) c3(transcription factor RFX3 isoform c) c4( ) c5(100307, 100308, 100309, 100310, 100311, 100312, 100313, 100314, 100315, 100316, 100317, 100318, 100319, 100320, 100321, 100322, 100323, 100324, 100325, 100326, 100327, 100328, 100329, 100330, 100331, 100332, 100333, 100334, 100335, 100336, 100337, 100338, 100339, 100340, 100341, 100342) c6( ) c7(Pk, j, lQ, awP); #c1(RFX3) c2(NP_002910) c3(transcription factor RFX3 isoform a) c4(133489, 133490, 133491, 133492, 133493, 133494, 133495) c5(133496, 133497, 133498, 133499, 133500, 133501, 133502, 133503, 133504, 133505) c6( ) c7(Pk, j, lQ, awP); #c1(RFX3) c2(NP_602304) c3(transcription factor RFX3 isoform b) c4(235685) c5(235686, 235687, 235688, 235689, 235690, 235691) c6( ) c7(Pk, j, lQ, awP); #c1(RFX3) c2(XP_006716903) c3(transcription factor RFX3 isoform X1) c4(290545, 290546) c5(290547) c6( ) c7(Pk, j, lQ, awP); #c1(RFX3) c2(XP_006716910) c3(transcription factor RFX3 isoform X5) c4( ) c5(290548, 290549, 290550, 290551, 290552, 290553, 290554, 290555, 290556, 290557, 290558, 290559, 290560, 290561, 290562, 290563, 290564) c6( ) c7(Pk, j, lQ, awP); #c1(RFX3) c2(XP_011516302) c3(transcription factor RFX3 isoform X3) c4(309510, 309511) c5(309512, 309513, 309514, 309515, 309516, 309517, 309518, 309519, 309520, 309521) c6( ) c7(Pk, j, lQ, awP); #c1(RFX4) c2(NP_001193620) c3(transcription factor RFX4 isoform d) c4(82366) c5(82367, 82368, 82369, 82370, 82371, 82372, 82373) c6( ) c7(bK); #c1(RFX4) c2(NP_115880) c3(transcription factor RFX4 isoform a) c4(224443) c5(224444, 224445, 224446, 224447, 224448, 224449) c6( ) c7(bK); #c1(RFX4) c2(NP_998759) c3(transcription factor RFX4 isoform c) c4(265861) c5(265862, 265863, 265864, 265865, 265866, 265867) c6( ) c7(bK); #c1(RFX5) c2(NP_001020774) c3(DNA-binding protein RFX5) c4(29029, 29030, 29031, 29032, 29033, 29034, 29035, 29036, 29037, 29038) c5(29039, 29040, 29041, 29042, 29043, 29044, 29045, 29046, 29047, 29048, 29049, 29050, 29051, 29052, 29053, 29054, 29055, 29056, 29057, 29058, 29059, 29060, 29061, 29062, 29063, 29064, 29065, 29066, 29067, 29068, 29069, 29070, 29071, 29072, 29073, 29074, 29075, 29076, 29077, 29078, 29079, 29080, 29081, 29082, 29083, 29084, 29085, 29086, 29087, 29088, 29089, 29090, 29091, 29092, 29093, 29094, 29095, 29096, 29097, 29098, 29099, 29100, 29101, 29102, 29103, 29104, 29105, 29106, 29107, 29108) c6( ) c7(dD, czh, W, ea, r, eq, Xa, bJR, WX, czg); #c1(RFX6) c2(NP_775831) c3(DNA-binding protein RFX6) c4(250469) c5(250470, 250471, 250472, 250473, 250474, 250475) c6( ) c7(mh, czi, Y, aj, ae, J, ad); #c1(RFX8) c2(NP_001139136) c3(DNA-binding protein RFX8) c4(58758, 58759) c5(58760, 58761, 58762, 58763, 58764, 58765, 58766, 58767, 58768, 58769, 58770, 58771) c6( ) c7(oe, pN); #c1(RFX8) c2(XP_011510074) c3(DNA-binding protein RFX8 isoform X2) c4(300691) c5(300692, 300693, 300694, 300695, 300696, 300697) c6( ) c7(oe, pN); #c1(RFXANK) c2(NP_001265656) c3(DNA-binding protein RFXANK isoform c) c4(99124) c5(99125, 99126, 99127, 99128, 99129, 99130, 99131, 99132, 99133, 99134, 99135, 99136) c6( ) c7(czj, WX, Xa, dA, CF); #c1(RFXANK) c2(NP_001265657) c3(DNA-binding protein RFXANK isoform b) c4(99137) c5(99138, 99139, 99140, 99141, 99142, 99143) c6( ) c7(czj, WX, Xa, dA, CF); #c1(RFXANK) c2(NP_003712) c3(DNA-binding protein RFXANK isoform a) c4(140358, 140359) c5(140360, 140361, 140362, 140363, 140364, 140365, 140366) c6( ) c7(czj, WX, Xa, dA, CF); #c1(RFXAP) c2(NP_000529) c3(regulatory factor X-associated protein) c4(11915) c5(11916, 11917, 11918, 11919, 11920, 11921, 11922) c6( ) c7(ac, WX, Xa, dA, czk); #c1(RGCC) c2(NP_054778) c3(regulator of cell cycle RGCC) c4(177768) c5(177769, 177770, 177771, 177772, 177773, 177774, 177775) c6( ) c7(T, jh, bB, j, ci, eA, hd, aE, aT, K, j, s, HI, aW, as, eE, t, bz, ck, es, bP, eM, nB, bE, cj, Hb, KS, X); #c1(RGL1) c2(NP_001284598) c3(ral guanine nucleotide dissociation stimulator-like 1 isoform 2) c4(112124) c5(112125, 112126, 112127, 112128, 112129, 112130) c6( ) c7(alv); #c1(RGL1) c2(NP_001284600) c3(ral guanine nucleotide dissociation stimulator-like 1 isoform 3) c4(112131) c5(112132, 112133, 112134, 112135, 112136, 112137) c6( ) c7(alv); #c1(RGL1) c2(NP_001284601) c3(ral guanine nucleotide dissociation stimulator-like 1 isoform 4) c4(112138) c5(112139, 112140, 112141, 112142, 112143, 112144) c6( ) c7(alv); #c1(RGL1) c2(NP_055964) c3(ral guanine nucleotide dissociation stimulator-like 1 isoform 1) c4(184717) c5(184718, 184719, 184720, 184721, 184722, 184723, 184724, 184725, 184726, 184727) c6( ) c7(alv); #c1(RGL1) c2(XP_011507644) c3(ral guanine nucleotide dissociation stimulator-like 1 isoform X3) c4(297587) c5(297588, 297589, 297590, 297591, 297592, 297593) c6( ) c7(alv); #c1(RGL2) c2(NP_004752) c3(ral guanine nucleotide dissociation stimulator-like 2 isoform 1) c4(149270, 149271) c5(149272, 149273, 149274, 149275, 149276, 149277, 149278, 149279, 149280, 149281, 149282, 149283, 149284, 149285, 149286, 149287, 149288, 149289, 149290, 149291, 149292, 149293, 149294, 149295, 149296, 149297, 149298, 149299, 149300, 149301, 149302, 149303, 149304, 149305, 149306, 149307, 149308, 149309, 149310) c6( ) c7(kp, eZ); #c1(RGL2) c2(XP_011513086) c3(ral guanine nucleotide dissociation stimulator-like 2 isoform X2) c4(304766, 304767) c5(304768, 304769, 304770, 304771, 304772, 304773, 304774, 304775, 304776, 304777, 304778, 304779, 304780, 304781, 304782, 304783, 304784) c6( ) c7(kp, eZ); #c1(RGL2) c2(XP_011513087) c3(ral guanine nucleotide dissociation stimulator-like 2 isoform X3) c4( ) c5(304785, 304786, 304787, 304788, 304789, 304790, 304791, 304792, 304793, 304794, 304795, 304796, 304797, 304798, 304799, 304800, 304801) c6( ) c7(kp, eZ); #c1(RGL4) c2(NP_705843) c3(ral-GDS-related protein precursor) c4(246351, 246352) c5(246353, 246354, 246355, 246356, 246357, 246358, 246359, 246360, 246361, 246362, 246363, 246364) c6( ) c7(db, cZ, T, dd); #c1(RGMA) c2(NP_001159755) c3(repulsive guidance molecule A isoform 1) c4(64739, 64740) c5(64741, 64742, 64743, 64744, 64745, 64746) c6( ) c7(eR, fm, oe, Z, aY, V); #c1(RGMA) c2(NP_001159761) c3(repulsive guidance molecule A isoform 2 precursor) c4(64766) c5(64767, 64768, 64769, 64770, 64771, 64772, 64773, 64774, 64775, 64776, 64777, 64778, 64779, 64780, 64781, 64782, 64783, 64784) c6( ) c7(eR, fm, oe, Z, aY, V); #c1(RGMA) c2(NP_064596) c3(repulsive guidance molecule A isoform 3) c4(203201) c5(203202, 203203, 203204, 203205, 203206, 203207, 203208, 203209) c6( ) c7(eR, fm, oe, Z, aY, V); #c1(RGMB) c2(NP_001012779) c3(RGM domain family member B) c4(25346, 25347) c5(25348, 25349, 25350, 25351, 25352, 25353) c6( ) c7(aW, y, aT); #c1(RGMB) c2(XP_005272023) c3(RGM domain family member B isoform X2) c4( ) c5(283660, 283661, 283662, 283663, 283664, 283665) c6( ) c7(aW, y, aT); #c1(RGMB) c2(XP_011541648) c3(RGM domain family member B isoform X3) c4( ) c5(340198) c6( ) c7(aW, y, aT); #c1(RGN) c2(NP_004674) c3(regucalcin isoform 1) c4(148730) c5(148731, 148732, 148733, 148734, 148735, 148736, 148737, 148738, 148739, 148740, 148741, 148742, 148743, 148744) c6( ) c7(T, amB, jp, aW, dQ, aT, fQ, aq, bS, dy, ct, bq, Z, ae, B, ad); #c1(RGN) c2(XP_006724630) c3(regucalcin isoform X1) c4(296147) c5(296148, 296149, 296150, 296151, 296152, 296153) c6( ) c7(T, amB, jp, aW, dQ, aT, fQ, aq, bS, dy, ct, bq, Z, ae, B, ad); #c1(RGPD2) c2(NP_001071638) c3(RANBP2-like and GRIP domain-containing protein 2) c4(35360, 35361, 35362, 35363) c5(35364, 35365, 35366, 35367, 35368, 35369, 35370, 35371, 35372, 35373, 35374, 35375, 35376, 35377, 35378, 35379, 35380, 35381) c6( ) c7(ck, T, iT, iU, h, bw, m, hf, hF, bt, br); #c1(RGPD2) c2(XP_011531391) c3(RANBP2-like and GRIP domain-containing protein 2 isoform X5) c4( ) c5(327390, 327391, 327392, 327393, 327394, 327395) c6( ) c7(ck, T, iT, iU, h, bw, m, hf, hF, bt, br); #c1(RGR) c2(NP_001012738) c3(RPE-retinal G protein-coupled receptor isoform 2) c4(25243) [5( ) c6( ) c7(cZ, lR, W, czl, nx, Pg, nw, dd, db, nC, VF); #c1(RGR) c2(NP_001012740) c3(RPE-retinal G protein-coupled receptor isoform 3) c4(25244) c5(25245, 25246, 25247, 25248, 25249, 25250, 25251, 25252) c6( ) c7(cZ, lR, W, czl, nx, Pg, nw, dd, db, nC, VF); #c1(RGR) c2(NP_002912) c3(RPE-retinal G protein-coupled receptor isoform 1) c4(133506) c5(133507, 133508, 133509, 133510, 133511, 133512, 133513, 133514, 133515, 133516) c6( ) c7(cZ, lR, W, czl, nx, Pg, nw, dd, db, nC, VF); #c1(RGS1) c2(NP_002913) c3(regulator of G-protein signaling 1) c4(133517) c5(133518, 133519, 133520, 133521, 133522, 133523) c6( ) c7(T, A, iM, BN, je, Y, fJ, ff, jh, kH, cL, hy, hp, lQ, en, eF, Pk, V, ne); #c1(RGS10) c2(NP_001005339) c3(regulator of G-protein signaling 10 isoform a) c4(21403, 21404) c5(21405, 21406, 21407, 21408, 21409, 21410, 21411, 21412, 21413, 21414, 21415, 21416) c6( ) c7(T, zw, BN, W, bE, ff, bB, hy, K, m, ajW, en, t, ne); #c1(RGS10) c2(NP_002916) c3(regulator of G-protein signaling 10 isoform b) c4(133538) c5(133539, 133540, 133541, 133542, 133543, 133544) c6( ) c7(T, zw, BN, W, bE, ff, bB, hy, K, m, ajW, en, t, ne); #c1(RGS11) c2(NP_003825) c3(regulator of G-protein signaling 11 isoform 2) c4(141177) c5( ) c6( ) c7(mR, BN, ff, hy, en, ne); #c1(RGS11) c2(NP_899180) c3(regulator of G-protein signaling 11 isoform 1) c4(257981) c5(257982, 257983, 257984, 257985, 257986, 257987, 257988, 257989, 257990, 257991, 257992, 257993) c6( ) c7(mR, BN, ff, hy, en, ne); #c1(RGS11) c2(XP_011521021) c3(regulator of G-protein signaling 11 isoform X1) c4( ) c5(315964) c6( ) c7(mR, BN, ff, hy, en, ne); #c1 (RGS11) c2(XP_011521025) c3(regulator of G-protein signaling 11 isoform X5) c4(315965) c5(315966, 315967, 315968, 315969, 315970, 315971) c6( ) c7(mR, BN, ff, hy, en, ne); #c1(RGS12) c2(NP_937870) c3(regulator of G-protein signaling 12 isoform 3) c4(259355) c5(259356, 259357, 259358, 259359, 259360, 259361) c6( ) c7(ac, BN, ff, hy, en, ne); #c1(RGS12) c2(XP_006713968) c3(regulator of G-protein signaling 12 isoform X1) c4(287436, 287437) c5(287438, 287439, 287440, 287441, 287442, 287443) c6( ) c7(ac, BN, ff, hy, en, ne); #c1(RGS12) c2(XP_006713969) c3(regulator of G-protein signaling 12 isoform X3) c4(287444, 287445) c5(287446, 287447, 287448, 287449, 287450, 287451, 287452, 287453, 287454, 287455, 287456, 287457, 287458, 287459, 287460, 287461, 287462, 287463) c6( ) c7(ac, BN, ff, hy, en, ne); #c1(RGS12) c2(XP_011511844) c3(regulator of G-protein signaling 12 isoform X2) c4(302510) c5(302511, 302512, 302513, 302514, 302515, 302516, 302517, 302518, 302519, 302520) c6( ) c7(ac, BN, ff, hy, en, ne); #c1(RGS13) c2(NP_002918) c3(regulator of G-protein signaling 13) c4(133545) c5(133546, 133547, 133548, 133549, 133550, 133551, 133552, 133553, 133554, 133555) c6( ) c7(BN, ff, jh, hy, en, Vk, ne); #c1(RGS14) c2(NP_006471) c3(regulator of G-protein signaling 14) c4(165452) c5(165453, 165454, 165455, 165456, 165457, 165458) c6( ) c7(BN, ff, hy, en, V, ne); #c1(RGS14) c2(XP_005265851) c3(regulator of G-protein signaling 14 isoform X1) c4(279790, 279791) c5(279792, 279793, 279794, 279795, 279796, 279797, 279798, 279799, 279800, 279801, 279802, 279803) c6( ) c7(BN, ff, hy, en, V, ne); #c1(RGS16) c2(NP_002919) c3(regulator of G-protein signaling 16) c4(133556) c5(133557, 133558, 133559, 133560, 133561, 133562) c6( ) c7(BN, aE, ne, ff, gX, aT, r, hy, ci, en, cm, q); #c1(RGS17) c2(XP_011534055) c3(regulator of G-protein signaling 17 isoform X3) c4(330233) c5(330234, 330235) c6( ) c7(av, aW, oe, al, at, cd, dL, m, t, bz, xa, ad); #c1(RGS17) c2(XP_011534056) c3(regulator of G-protein signaling 17 isoform X1) c4( ) c5(330236, 330237, 330238, 330239, 330240, 330241, 330242, 330243) c6( ) c7(av, aW, oe, al, at, cd, dL, m, t, bz, xa, ad); #c1 (RGS18) c2(NP_570138) c3(regulator of G-protein signaling 18) c4(234050) c5(234051, 234052, 234053, 234054, 234055, 234056) c6( ) c7(BN, ff, jh, hy, en, Vk, ne); #c1(RGS19) c2(XP_011526788) c3(regulator of G-protein signaling 19 isoform X1) c4(321064) c5(321065, 321066, 321067, 321068, 321069, 321070, 321071, 321072, 321073, 321074) c6( ) c7(bB, bE); #c1(RGS2) c2(NP_002914) c3(regulator of G-protein signaling 2) c4(133524) c5(133525, 133526, 133527, 133528, 133529, 133530) c6( ) c7(gh, dD, ea, oe, e, eX, aA, cz, n, akA, bS, hy, v, eq, ww, V, q, ad, bAO, QU, aT, ta, eV, dL, K, bBp, an, dQ, ae, eL, czm, BN, tb, ff, r, en, as, JI, t, jb, qa, hq, bdn, rR, yP, sv, cS, ei, bt, ne); #c1(RGS20) c2(NP_003693) c3(regulator of G-protein signaling 20 isoform b) c4(140201) c5(140202, 140203, 140204, 140205, 140206, 140207) c6( ) c7(BN, ff, cz, hy, en, ne); #c1(RGS20) c2(NP_733466) c3(regulator of G-protein signaling 20 isoform a) c4(246888) c5(246889, 246890, 246891, 246892, 246893, 246894) c6( ) c7(BN, ff, cz, hy, en, ne); #c1(RGS20) c2(XP_011515924) c3 (regulator of G-protein signaling 20 isoform X1) c4( ) c5(309074) c6( ) c7(BN, ff, cz, hy, en, ne); #c1(RGS21) c2(NP_001034241) c3(regulator of G-protein signaling 21) c4(31636) c5(31637, 31638, 31639, 31640, 31641, 31642) c6( ) c7(xY, hp); #c1(RGS22) c2(NP_001273621) c3(regulator of G-protein signaling 22 isoform 2) c4(105488) c5(105489, 105490, 105491, 105492, 105493, 105494) c6( ) c7(T, sS, BN, ff, ht, hy, en, hu, ne); #c1(RGS22) c2(NP_056483) c3(regulator of G-protein signaling 22 isoform 1) c4(187715) c5(187716, 187717, 187718, 187719, 187720, 187721) c6( ) c7(T, sS, BN, ff, ht, hy, en, hu, ne); #c1(RGS3) c2(NP_001263190) c3(regulator of G-protein signaling 3 isoform 7) c4(96985) c5(96986, 96987, 96988, 96989, 96990, 96991) c6( ) c7(BN, ne, ff, hy, eA, en, cd); #c1(RGS3) c2(NP_060260) c3(regulator of G-protein signaling 3 isoform 3) c4(195102) c5(195103, 195104, 195105, 195106, 195107, 195108) c6( ) c7(BN, ne, ff, hy, eA, en, cd); #c1(RGS3) c2(NP_570613) c3(regulator of G-protein signaling 3 isoform 1) c4(234121) c5(234122, 234123, 234124, 234125, 234126, 234127) c6( ) c7(BN, ne, ff, hy, eA, en, cd); #c1(RGS3) c2(NP_602299) c3(regulator of G-protein signaling 3 isoform 4) c4( ) c5(235680, 235681, 235682, 235683, 235684) c6( ) c7(BN, ne, ff, hy, eA, en, cd); #c1(RGS3) c2(NP_652759) c3(regulator of G-protein signaling 3 isoform 6) c4(238805) c5(238806, 238807, 238808, 238809, 238810, 238811, 238812, 238813, 238814, 238815) c6( ) c7(BN, ne, ff, hy, eA, en, cd); #c1(RGS3) c2(NP_652760) c3(regulator of G-protein signaling 3 isoform 5) c4(238816) c5(238817, 238818, 238819, 238820, 238821, 238822, 238823) c6( ) c7(BN, ne, ff, hy, eA, en, cd); #c1(RGS3) c2(XP_006717285) c3(regulator of G-protein signaling 3 isoform X4) c4(290880) c5(290881) c6( ) c7(BN, ne, ff, hy, eA, en, cd); #c1(RGS3) c2(XP_006717287) c3(regulator of G-protein signaling 3 isoform X7) c4(290882) c5( ) c6( ) c7(BN, ne, ff, hy, eA, en, cd); #c1(RGS3) c2(XP_006717293) c3(regulator of G-protein signaling 3 isoform X1) c4( ) c5(290883, 290884, 290885, 290886, 290887, 290888, 290889, 290890, 290891) c6( ) c7(BN, ne, ff, hy, eA, en, cd); #c1(RGS4) c2(NP_001095915) c3(regulator of G-protein signaling 4 isoform 1) c4(40933, 40934, 40935, 40936) c5(40937, 40938, 40939, 40940, 40941, 40942, 40943, 40944) c6( ) c7(iH, bO, cz, ti, hy, bS, cd, eA, kL, dP, aT, bdp, dL, bK, ey, he, eL, af, bvX, BN, aW, ff, gl, Ku, en, cm, jb, sv, ne); #c1(RGS4) c2(NP_001106851) c3(regulator of G-protein signaling 4 isoform 3) c4( ) c5(42624, 42625, 42626, 42627, 42628, 42629, 42630, 42631, 42632, 42633, 42634, 42635, 42636) c6( ) c7(iH, bO, cz, ti, hy, bS, cd, eA, kL, dP, aT, bdp, dL, bK, ey, he, eL, af, bvX, BN, aW, ff, gl, Ku, en, cm, jb, sv, ne); #c1(RGS4) c2(NP_001106852) c3(regulator of G-protein signaling 4 isoform 4) c4(42637) c5(42638, 42639, 42640, 42641, 42642, 42643) c6( ) c7(iH, bO, cz, ti, hy, bS, cd, eA, kL, dP, aT, bdp, dL, bK, ey, he, el, af, bvX, BN, aW, ff, gl, Ku, en, cm, jb, sv, ne); #c1(RGS4) c2(NP_005604) c3(regulator of G-protein signaling 4 isoform 2) c4( ) c5(157890, 157891, 157892, 157893, 157894, 157895, 157896) c6( ) c7(iH, bO, cz, ti, hy, bS, cd, eA, kL, dP, aT, bdp, dL, bK, ey, he, eL, af, bvX, BN, aW, ff, gl, Ku, en, cm, jb, sv, ne); #c1(RGS5) c2(NP_001241677) c3(regulator of G-protein signaling 5 isoform 2) c4( ) c5(89036, 89037, 89038, 89039, 89040) c6( ) c7(T, dD, cz, n, hy, eq, fr, cd, dL, bK, B, BN, Y, ff, aq, en, iR, HI, qa, YE, iA, iS, at, m, ne); #c1(RGS5) c2(NP_003608) c3(regulator of G-protein signaling 5 isoform 1) c4(139550, 139551, 139552) c5(139553, 139554, 139555, 139556, 139557, 139558, 139559, 139560, 139561, 139562, 139563, 139564, 139565) c6( ) c7(T, dD, cz, n, hy, eq, fr, cd, dL, bK, B, BN, Y, ff, aq, en, iR, Hl, qa, YE, iA, iS, at, m, ne); #c1(RGS6) c2(NP_001191346) c3(regulator of G-protein signaling 6 isoform 3) c4(81431, 81432, 81433) c5(81434, 81435, 81436, 81437, 81438, 81439, 81440, 81441, 81442, 81443, 81444, 81445, 81446, 81447, 81448, 81449, 81450, 81451) c6( ) c7(T, hl, WA, cz, KO, v, fr, kv, nA, Fc, ad, UE, iu, aE, aT, hD, dL, hP, K, zy, ae, cjs, eL, cxV, iU, aW, r, as, bQ, eE, bz, dr, es, Ew, eM, lq, at, bt, B, afH); #c1(RGS6) c2(NP_001191348) c3(regulator of G-protein signaling 6 isoform 5) c4(81452) c5(81453, 81454, 81455, 81456, 81457, 81458) c6( ) c7(T, hl, WA, cz, KO, v, fr, kv, nA, Fc, ad, UE, iu, aE, aT, hD, dL, hP, K, zy, ae, cjs, eL, cxV, iU, aW, r, as, bl, eE, bz, dr, es, Ew, eM, lq, at, bt, B, afH); #c1(RGS6) c2(NP_001191353) c3(regulator of G-protein signaling 6 isoform 1) c4(81459, 81460, 81461) c5(81462, 81463, 81464, 81465, 81466, 81467, 81468, 81469, 81470, 81471, 81472) c6( ) c7(T, hl, WA, cz, KO, v, fr, kv, nA, Fc, ad, UE, iu, aE, aT, hD, dL, hP, K, zy, ae, cjs, eL, cxV, iU, aW, r, as, bQ, eE, bz, dr, es, Ew, eM, lq, at, bt, B, afH); #c1(RGS6) c2(XP_005268292) c3(regulator of G-protein signaling 6 isoform X18) c4( ) c5(281676, 281677, 281678, 281679, 281680, 281681) c6( ) c7(T, hl, WA, cz, KO, v, fr, kv, nA, Fc, ad, UE, iu, aE, aT, hD, dL, hP, K, zy, ae, cjs, eL, cxV, iU, aW, r, as, bQ, eE, bz, dr, es, Ew, eM, lq, at, bt, B, afH); #c1(RGS6) c2(XP_005268293) c3(regulator of G-protein signaling 6 isoform X1) c4(281682) c5(281683, 281684, 281685, 281686, 281687, 281688, 281689, 281690, 281691, 281692, 281693, 281694, 281695, 281696) c6( ) c7(T, hl, WA, cz, KO, v, fr, kv, nA, Fc, ad, UE, iu, aE, aT, HD, dL, hP, K, zy, ae, cjs, eL, cxV, iU, aW, r, as, bQ, eE, bz, dr, es, Ew, eM, lq, at, bt, B, afH); #c1(RGS6) c2(XP_011535691) c3(regulator of G-protein signaling 6 isoform X3) c4(332921, 332922) c5(332923, 332924, 332925, 332926, 332927, 332928, 332929, 332930, 332931, 332932, 332933, 332934) c6( ) c7(T, hl, WA, cz, KO, v, fr, kv, nA, Fc, ad, UE, iu, aE, aT, HD, dL, hP, K, zy, ae, cjs, eL, cxV, iU, aW, r, as, bd, eE, bz, dr, es, Ew, eM, lq, at, bt, B, afH); #c1(RGS6) c2(XP_011535710) c3(regulator of G-protein signaling 6 isoform X20) c4(332935, 332936) c5(332937, 332938, 332939, 332940, 332941, 332942, 332943, 332944, 332945, 332946, 332947, 332948, 332949, 332950, 332951, 332952, 332953) c6( ) c7(T, hl, WA, cz, KO, v, fr, kv, nA, Fc, ad, UE, iu, aE, aT, hD, dL, hP, K, zy, ae, cjs, eL, cxV, iU, aW, r, as, bQ, BE, bz, dr, es, Ew, eM, lq, at, bt, B, afH); #c1(RGS7) c2(NP_001269702) c3(regulator of G-protein signaling 7 isoform 2) c4(102614) c5(102615, 102616, 102617, 102618, 102619, 102620, 102621, 102622, 102623, 102624) c6( ) c7(BN, tb, ff, hy, ww, j, nA, en, V, ne); #c1(RGS7) c2(NP_001269704) c3(regulator of G-protein signaling 7 isoform 3) c4(102637, 102638, 102639) c5(102640, 102641, 102642, 102643, 102644, 102645, 102646, 102647, 102648, 102649, 102650, 102651, 102652, 102653, 102654, 102655, 102656, 102657) c6( ) c7(BN, tb, ff, hy, ww, j, nA, en, V, ne); #c1(RGS7) c2(NP_001269707) c3(regulator of G-protein signaling 7 isoform 4) c4(102665) c5(102666, 102667, 102668, 102669, 102670, 102671, 102672, 102673, 102674, 102675) c6( ) c7(BN, tb, ff, hy, ww, j, nA, en, V, ne); #c1(RGS7) c2(NP_002915) c3(regulator of G-protein signaling 7 isoform 1) c4(133531) c5(133532, 133533, 133534, 133535, 133536, 133537) c6( ) c7(BN, tb, ff, hy, ww, j, nA, en, V, ne); #c1(RGS7) c2(XP_005273275) c3(regulator of G-protein signaling 7 isoform X1) c4(284418, 284419) c5(284420, 284421, 284422, 284423, 284424, 284425) c6( ) c7(BN, tb, ff, hy, ww, j, nA, en, V, ne); #c1(RGS7) c2(XP_005273278) c3(regulator of G-protein signaling 7 isoform X2) c4( ) c5(284426, 284427, 284428, 284429, 284430, 284431) c6( ) c7(BN, tb, ff, hy, ww, j, nA, en, V, ne); #c1(RGS7BP) c2(NP_001025046) c3(regulator of G-protein signaling 7-binding protein isoform 1) c4(29267) c5(29268, 29269, 29270, 29271, 29272, 29273) c6( ) c7(aDy, bM, tk); #c1(RGS8) c2(XP_005245612) c3(regulator of G-protein signaling 8 isoform X1) c4(266767, 266768) c5(266769, 266770, 266771, 266772, 266773, 266774, 266775, 266776, 266777, 266778, 266779, 266780, 266781, 266782, 266783, 266784) c6( ) c7(BN, Ub, ff, hy, en, ne); #c1(RGS8) c2(XP_011508391) c3(regulator of G-protein signaling 8 isoform X2) c4(299076) c5(299077, 299078, 299079, 299080, 299081, 299082, 299083, 299084, 299085, 299086) c6( ) c7(BN, Ub, ff, hy, en, ne); #c1(RGS9) c2(NP_001075424) c3(regulator of G-protein signaling 9 isoform 2) c4(37272) c5(37273, 37274, 37275, 37276, 37277, 37278) c6( ) c7(HF, dD, BN, ac, aw, czn, ff, sv, hy, eq, HL, en, HH, cm, ne, cv); #c1(RGS9) c2(NP_003826) c3(regulator of G-protein signaling 9 isoform 1) c4(141178) c5(141179, 141180, 141181, 141182, 141183, 141184) c6( ) c7(HF, dD, BN, ac, aw, czn, ff, sv, hy, eq, HL, en, HH, cm, ne, cv); #c1(RGS9BP) c2(NP_997274) c3(regulator of G-protein signaling 9-binding protein) c4(265367) c5(265368, 265369, 265370, 265371, 265372, 265373, 265374) c6( ) c7(HF, dD, KE, czn, HL, eq, HH); #c1(RGSL1) c2(NP_001131141) c3(regulator of G-protein signaling protein-like) c4(52615) c5(52616, 52617, 52618, 52619, 52620, 52621) c6( ) c7(aW, af, aT); #c1(RHAG) c2(NP_000315) c3(ammonium transporter Rh type A) c4(6970, 6971, 6972, 6973) c5(6974, 6975, 6976, 6977, 6978, 6979, 6980, 6981, 6982, 6983, 6984, 6985, 6986, 6987, 6988, 6989, 6990, 6991, 6992, 6993, 6994, 6995, 6996, 6997) c6( ) c7(czp, uu, czo, cfR); #c1(RHBDD2) c2(NP_001035546) c3(rhomboid domain-containing protein 2 isoform a) c4(32916, 32917, 32918) c5(32919, 32920, 32921, 32922, 32923, 32924, 32925, 32926, 32927, 32928, 32929, 32930, 32931, 32932, 32933, 32934) c6(32935, 32936) c7(aW, r, al, n, K, q); #c1(RHBDD2) c2(NP_001035547) c3(rhomboid domain-containing protein 2 isoform b) c4( ) c5(32937, 32938, 32939, 32940, 32941, 32942, 32943, 32944, 32945, 32946, 32947) c6( ) c7(aW, r, aT, n, K, q); #c1(RHBDD3) c2(NP_036397) c3(rhomboid domain-containing protein 3) c4(173587, 173588, 173589) c5(173590, 173591, 173592, 173593, 173594, 173595, 173596, 173597, 173598, 173599, 173600, 173601, 173602, 173603, 173604, 173605, 173606, 173607) c6( ) c7(hR, lB); #c1(RHBDF1) c2(NP_071895) c3(inactive rhomboid protein 1) c4(212120, 212121, 212122) c5(212123, 212124, 212125, 212126, 212127, 212128, 212129, 212130, 212131, 212132, 212133, 212134) c6( ) c7(T, aE, bE, aW, oV, qt, ff, aA, r, aT, bB, af, en, g, q); #c1 (RHBDF1) c2(XP_005255555) c3(inactive rhomboid protein 1 isoform X6) c4( ) c5(273731, 273732, 273733, 273734, 273735, 273736) c6( ) c7(T, aE, bE, aW, oV, qt, ff, aA, r, aT, bB, aF, en, g, q); #c1(RHBDF1) c2(XP_011520900) c3(inactive rhomboid protein 1 isoform X3) c4(315891) c5( ) c6( ) c7(T, aE, bE, aW, oV, qt, ff, aA, r, aT, bB, aF, en, g, q); #c1(RHBDF1) c2(XP_011520901) c3(inactive rhomboid protein 1 isoform X4) c4(315892) c5( ) c6( ) c7(T, aE, bE, aW, oV, qt, ff, aA, r, aT, bB, aF, en, g, q); #c1(RHBDF1) c2(XP_011520902) c3(inactive rhomboid protein 1 isoform X5) c4( ) c5(315893, 315894, 315895, 315896, 315897, 315898, 315899, 315900, 315901, 315902, 315903, 315904) c6( ) c7(T, aE, bE, aW, oV, qt, ff, aA, r, aT, bB, aF, en, g, q); #c1(RHBDF2) c2(NP_001005498) c3(inactive rhomboid protein 2 isoform 2) c4(21755, 21756) c5(21757, 21758, 21759, 21760, 21761, 21762, 21763, 21764, 21765, 21766, 21767, 21768) c6( ) c7(T, kh, aNA, ht, fr, ci, pd, ai, br, aT, bCv, hu, hM, aW, ff, baP, en, cm, t, bw, m, czq, B); #c1(RHBDF2) c2(NP_078875) c3(inactive rhomboid protein 2 isoform 1) c4(215869) c5(215870, 215871, 215872, 215873, 215874, 215875) c6( ) c7(T, kh, aNA, ht, fr, ci, pd, ai, br, aT, bCv, hu, hM, aW, ff, baP, en, cm, t, bw, m, czq, B); #c1(RHBDL1) c2(NP_001265649) c3(rhomboid-related protein 1 isoform 1) c4(99109, 99110) c5(99111, 99112, 99113, 99114, 99115, 99116, 99117, 99118, 99119, 99120, 99121, 99(22) c6( ) c7(n, cd); #c1(RHBDL2) c2(NP_001291675) c3(rhomboid-related protein 2 isoform 1) c4(116312, 116313) c5(116314, 116315, 116316, 116317, 116318, 116319, 116320, 116321, 116322, 116323, 116324, 116325, 116326, 116327) c6( ) c7(gv, ad); #c1(RHCE) c2(XP_011540191) c3(blood group Rh(CE) polypeptide isoform X2) c4( ) c5(338586, 338587, 338588, 338589, 338590, 338591, 338592, 338593, 338594, 338595) c6( ) c7(atU, qb, Y, yk, qd, dV, czr, qu, CI, xL, czs, kL); #c1(RHCE) c2(XP_011540192) c3(blood group Rh(CE) polypeptide isoform X3) c4(338596) c5(338597, 338598, 338599, 338600, 338601, 338602) c6( ) c7(atU, qb, Y, yk, qd, dV, czr, qu, CI, xL, czs, kL); #c1(RHCG) c2(XP_011519965) c3 (ammonium transporter Rh type C isoform X1) c4(314799, 314800, 314801, 314802) c5(314803, 314804, 314805, 314806, 314807, 314808, 314809, 314810, 314811, 314812, 314813, 314814, 314815, 314816, 314817, 314818, 314819, 314820, 314821, 314822, 314823, 314824, 314825, 314826) c6( ) c7(Z); #c1(RHEB) c2(NP_005605) c3(GTP-binding protein Rheb) c4(157897) c5(157898, 157899, 157900, 157901, 157902, 157903) c6( ) c7(hl, Ew, eM, Y, jh, aj, dG, dL, ci, Dz, ae, J, ad); #c1(RHEBL1) c2(NP_653194) c3(GTPase RhebL1 isoform 1 precursor) c4(239000) c5(239001, 239002, 239003, 239004, 239005, 239006) c6( ) c7(py); #c1(RHNO1) c2(NP_001239428) c3(RAD9, HUS1, RAD1-interacting nuclear orphan protein 1 isoform 1) c4(88424, 88425, 88426, 88427) c5(88428, 88429, 88430, 88431, 88432, 88433, 88434, 88435, 88436, 88437, 88438, 88439, 88440, 88441, 88442, 88443, 88444, 88445, 88446, 88447, 88448, 88449, 88450, 88451) c6( ) c7(aW, aT); #c1(RHNO1) c2(NP_001239429) c3(RAD9, HUS1, RAD1-interacting nuclear orphan protein 1 isoform 2) c4(88452) c5(88453, 88454, 88455, 88456, 88457, 88458) c6( ) c7(aW, aT); #c1(RHO) c2(NP_000530) c3(rhodopsin) c4(11923) c5(11924, 11925, 11926, 11927, 11928, 11929, 11930, 11931, 11932, 11933, 11934, 11935, 11936, 11937, 11938, 11939, 11940, 11941, 11942, 11943, 11944, 11945, 11946, 11947, 11948, 11949, 11950, 11951, 11952, 11953, 11954, 11955, 11956, 11957, 11958, 11959, 11960, 11961, 11962, 11963, 11964, 11965, 11966, 11967, 11968, 11969, 11970, 11971, 11972, 11973, 11974, 11975, 11976, 11977, 11978, 11979, 11980, 11981, 11982, 11983, 11984, 11985, 11986, 11987, 11988, 11989, 11990, 11991, 11992, 11993, 11994, 11995, 11996, 11997, 11998, 11999, 12000, 12001, 12002, 12003, 12004, 12005, 12006, 12007, 12008, 12009, 12010, 12011, 12012, 12013, 12014, 12015, 12016, 12017, 12018) c6(12019, 12020, 12021) c7(T, nx, fZ, ah, ci, fr, czw, cap, ad, afR, aT, czu, K, bFs, ae, AT, xX, d, lR, apo, MD, Y, tv, aq, tu, g, nk, iR, t, nC, cm, ayj, dr, bN, bgg, y, W, nb, ac, dB, bnP, eM, bP, czt, czv, cj, U, vy, nw, agf, m, Bd, B, ao, de); #c1(RHOB) c2(NP_004031) c3(rho-related GTP-binding protein RhoB precursor) c4(142956) c5(142957, 142958, 142959, 142960, 142961, 142962) c6( ) c7(pb, av, T, iu, d, nB, aW, Y, ff, aT, r, aA, czx, K, m, ci, q, t, en, B, ad); #c1(RHOBTB1) c2(XP_011538726) c3(rho-related BTB domain-containing protein 1 isoform X1) c4(336477) c5(336478, 336479, 336480, 336481, 336482, 336483, 336484, 336485, 336486, 336487) c6( ) c7(bB, bE, ff, en); #c1(RHOBTB1) c2(XP_011538727) c3(rho-related BTB domain-containing protein 1 isoform X2) c4(336488) c5( ) c6( ) c7(bB, bE, ff, en); #c1(RHOBTB1) c2(XP_011538730) c3(rho-related BTB domain-containing protein 1 isoform X5) c4( ) c5(336489, 336490, 336491, 336492, 336493, 336494) c6( ) c7(bB, bE, ff, en); #c1(RHOBTB2) c2(NP_001153508) c3(rho-related BTB domain-containing protein 2 isoform 1) c4(60630) c5(60631, 60632, 60633, 60634, 60635, 60636, 60637) c6( ) c7(qw, T, qt, aW, Pg, aA, aT, cz, fd, dL, aF, ci, bX, czy, iK, eL, ai); #c1(RHOBTB2) c2(NP_001153509) c3(rho-related BTB domain-containing protein 2 isoform 2) c4(60638) c5(60639, 60640, 60641, 60642, 60643, 60644, 60645, 60646, 60647, 60648, 60649) c6( ) c7(qw, T, qt, aW, Pg, aA, aT, cz, fd, dL, aF, ci, bX, czy, iK, eL, ai); #c1(RHOBTB2) c2(XP_011542756) c3(rho-related BTB domain-containing protein 2 isoform X2) c4(341389) c5( ) c6( ) c7(qw, T, qt, aW, Pg, aA, aT, cz, fd, dL, aF, ci, bX, czy, iK, eL, ai); #c1(RHOBTB3) c2(NP_055714) c3(rho-related BTB domain-containing protein 3) c4(183009) c5(183010, 183011, 183012, 183013, 183014, 183015) c6( ) c7(aj, oe); #c1(RHOBTB3) c2(XP_011541581) c3(rho-related BTB domain-containing protein 3 isoform X1) c4(340118) c5(340119, 340120, 340121, 340122, 340123, 340124) c6( ) c7(aj, oe); #c1(RHOD) c2(NP_055393) c3(rho-related GTP-binding protein RhoD isoform 1) c4(180746) c5(180747, 180748, 180749, 180750, 180751, 180752) c6( ) c7(T, fZ, n, ah, ci, fr, ad, afR, aT, K, ae, xX, d, MD, aW, Y, tv, aq, tu, g, nk, iR, t, nC, bN, ac, eM, dB, cj, agf, m, y, B); #c1 (RHOF) c2(NP_061907) c3(rho-related GTP-binding protein RhoF precursor) c4(201140, 201141) c5(201142, 201143, 201144, 201145, 201146, 201147, 201148, 201149, 201150, 201151, 201152, 201153) c6( ) c7(ccn, bcb, ig, xR, dV, CH, h, jh, bDI, eh, ciP, LH); #c1(RHOG) c2(XP_005252973) c3(rho-related GTP-binding protein RhoG isoform X1) c4(272088) c5(272089, 272090, 272091, 272092, 272093, 272094, 272095, 272096, 272097, 272098, 272099, 272100, 272101, 272102, 272103, 272104, 272105, 272106) c6( ) c7(h); #c1(RHOH) c2(NP_001265298) c3(rho-related GTP-binding protein RhoH precursor) c4(98191, 98192, 98193, 98194) c5(98195, 98196, 98197, 98198, 98199, 98200, 98201, 98202, 98203, 98204, 98205, 98206, 98207, 98208, 98209, 98210, 98211, 98212, 98213, 98214, 98215, 98216, 98217, 98218, 98219, 98220, 98221) c6( ) c7(bM, pa, fM, zK, CM, eX, jh, cj, hD, eV, fd, i, bt, zn); #c1(RHOJ) c2(NP_065714) c3(rho-related GTP-binding protein RhoJ precursor) c4(205049) c5(205050, 205051, 205052, 205053, 205054, 205055) c6( ) c7(auv, T, cc, beE, jh, oX, pz, X); #c1(RHOQ) c2(NP_036381) c3(rho-related GTP-binding protein Rhod precursor) c4(173425) c5(173426, 173427, 173428, 173429, 173430, 173431) c6( ) c7(bL, T, q, r); #c1(RHOT1) c2(NP_001028738) c3(mitochondrial Rho GTPase 1 isoform 2) c4(30589) c5(30590, 30591, 30592, 30593, 30594, 30595) c6( ) c7(c, an); #c1(RHOT1) c2(NP_001028740) c3(mitochondrial Rho GTPase 1 isoform 1) c4(30596) c5(30597, 30598, 30599, 30600, 30601, 30602) c6( ) c7(c, an); #c1(RHOT1) c2(NP_001275683) c3(mitochondrial Rho GTPase 1 isoform 5) c4(107039, 107040) c5(107041, 107042, 107043, 107044, 107045, 107046) c6( ) c7(c, an); #c1(RHOT1) c2(NP_060777) c3(mitochondrial Rho GTPase 1 isoform 3) c4(197734) c5(197735, 197736, 197737, 197738, 197739, 197740, 197741, 197742, 197743, 197744, 197745, 197746) c6( ) c7(c, an); #c1(RHOU) c2(NP_067028) c3(rho-related GTP-binding protein RhoU) c4(208532) c5(208533, 208534, 208535, 208536, 208537, 208538) c6( ) c7(dP, T, hw, aW, hk, Y, afQ, ff, aT, aA, aF, en, an, cd); #c1(RHOV) c2(NP_598378) c3(rho-related GTP-binding protein RhoV) c4(235475) c5(235476, 235477, 235478, 235479, 235480, 235481) c6( ) c7(cm, sk, Ou, ya); #c1(RHOXF1) c2(NP_644811) c3(rhox homeobox family member 1) c4( ) c5(238576, 238577, 238578, 238579, 238580) c6( ) c7(T); #c1(RHOXF1) c2(XP_011529583) c3(rhox homeobox family member 1 isoform X1) c4(325141) c5(325142) c6( ) c7(T); #c1(RHOXF2) c2(NP_115887) c3(rhox homeobox family member 2) c4(224533) c5(224534, 224535, 224536, 224537, 224538, 224539) c6( ) c7(ck, T, af, aA); #c1(RHPN1) c2(NP_443156) c3(rhophilin-1) c4(229670) c5(229671, 229672, 229673, 229674, 229675, 229676) c6( ) c7(kl); #c1(RHPN2) c2(NP_149094) c3(rhophilin-2) c4(227110, 227111, 227112) c5(227113, 227114, 227115, 227116, 227117, 227118, 227119, 227120, 227121, 227122, 227123, 227124, 227125, 227126, 227127, 227128, 227129, 227130) c6( ) c7(d, eA, r); #c1(RIBC2) c2(NP_056468) c3(RIB43A-like with coiled-coils protein 2) c4(187646, 187647) c5(187648, 187649, 187650, 187651, 187652, 187653, 187654) c6( ) c7(aT); #c1(RIBC2) c2(XP_005261581) c3(RIB43A-like with coiled-coils protein 2 isoform X1) c4( ) c5(277492, 277493, 277494, 277495, 277496) c6( ) c7(aT); #c1(RIC1) c2(NP_001129392) c3(RAB6A-GEF complex partner protein 1 isoform b) c4(51412, 51413) c5(51414, 51415, 51416, 51417, 51418, 51419, 51420, 51421, 51422, 51423, 51424, 51425) c6( ) c7(an); #c1(RIC1) c2(NP_001193486) c3(RAB6A-GEF complex partner protein 1 isoform c) c4(82224) c5(82225, 82226, 82227, 82228, 82229, 82230) c6( ) c7(an); #c1(RIC1) c2(NP_065880) c3(RAB6A-GEF complex partner protein 1 isoform a) c4(206051, 206052) c5(206053, 206054, 206055, 206056, 206057, 206058, 206059, 206060, 206061, 206062, 206063, 206064) c6( ) c7(an); #c1(RIC3) c2(NP_001128581) c3(protein RIC-3 isoform b precursor) c4(50329) c5(50330, 50331, 50332, 50333, 50334, 50335) c6( ) c7(cm, V); #c1(RIC3) c2(NP_001193600) c3(protein RIC-3 isoform c precursor) c4(82321, 82322) c5(82323, 82324, 82325, 82326, 82327, 82328, 82329, 82330, 82331, 82332, 82333, 82334, 82335, 82336, 82337, 82338, 82339, 82340) c6( ) c7(cm, V); #c1(RIC3) c2(NP_001193601) c3(protein RIC-3 isoform d precursor) c4(82341) c5(82342, 82343, 82344, 82345, 82346, 82347) c6( ) c7(cm, V); #c1(RIC3) c2(NP_078833) c3(protein RIC-3 isoform a precursor) c4(215647) c5(215648, 215649, 215650, 215651, 215652, 215653) c6( ) c7(cm, V); #c1(RIC3) c2(XP_006718380) c3 (protein RIC-3 isoform X2) c4(291800, 291801, 291802) c5(291803, 291804, 291805, 291806, 291807, 291808) c6( ) c7(cm, V); #c1(RIC3) c2(XP_006718384) c3(protein RIC-3 isoform X4) c4( ) c5(291809, 291810, 291811, 291812, 291813, 291814, 291815) c6( ) c7(cm, V); #c1(RICTOR) c2(NP_001272368) c3(rapamycin-insensitive companion of mTOR isoform 2) c4(104006) c5(104007, 104008, 104009, 104010, 104011, 104012) c6( ) c7(jf, BN, fR, ne, hW, n, hy, eA, g, ae, B, ad, aBs); #c1(RICTOR) c2(NP_689969) c3(rapamycin-insensitive companion of mTOR isoform 1) c4(244631) c5(244632, 244633, 244634, 244635, 244636, 244637) c6( ) c7(jf, BN, fR, ne, hW, n, hy, eA, g, ae, B, ad, aBs); #c1(RIF1) c2(NP_001171134) c3(telomere-associated protein RIF1 isoform 2) c4(69916) c5(69917, 69918, 69919, 69920, 69921, 69922, 69923, 69924, 69925, 69926, 69927, 69928, 69929, 69930) c6( ) c7(eG, cF); #c1(RIF1) c2(NP_060621) c3(telomere-associated protein RIF1 isoform 1) c4(196861) c5(196862, 196863, 196864, 196865, 196866, 196867, 196868, 196869, 196870, 196871, 196872, 196873, 196874, 196875, 196876) c6( ) c7(eG, cF); #c1(RIF1) c2(XP_011509696) c3 (telomere-associated protein RIF1 isoform X3) c4(300263, 300264) c5( ) c6( ) c7(eG, cF); #c1(RIF1) c2(XP_011509697) c3(telomere-associated protein RIF1 isoform X4) c4( ) c5(300265, 300266, 300267, 300268, 300269, 300270, 300271) c6( ) c7(eG, cF); #c1(RILP) c2(NP_113618) c3(rab-interacting lysosomal protein) c4(221753) c5(221754, 221755, 221756, 221757, 221758, 221759) c6( ) c7(czz); #c1(RILPL1) c2(NP_847884) c3(RILP-like protein 1) c4(253643) c5(253644, 253645, 253646, 253647, 253648, 253649) c6( ) c7(oe); #c1(RIMBP2) c2(NP_056162) c3(RIMS-binding protein 2) c4(186054) c5(186055, 186056, 186057, 186058, 186059) c6( ) c7(bP, eM, af, aA, aF, bz, B); #c1(RIMBP2) c2(XP_011536407) c3(RIMS-binding protein 2 isoform X3) c4( ) c5(333714) c6( ) c7(bP, eM, af, aA, aF, bz, B); #c1(RIMBP2) c2(XP_011536409) c3(RIMS-binding protein 2 isoform X5) c4(333715, 333716) c5(333717, 333718, 333719, 333720, 333721, 333722, 333723, 333724, 333725, 333726, 333727, 333728) c6( ) c7(bP, eM, af, aA, aF, bz, B); #c1(RIMBP3C) c2(NP_001122105) c3(RIMS-binding protein 3C) c4(46442, 46443) c5(46444, 46445, 46446, 46447, 46448, 46449, 46450, 46451, 46452, 46453, 46454, 46455, 46456, 46457) c6( ) c7(eZ); #c1(RIMS1) c2(NP_001161879) c3(regulating synaptic membrane exocytosis protein 1 isoform 2) c4(66276) c5(66277, 66278, 66279, 66280, 66281, 66282) c6( ) c7(IR, nb, W, nx, aVu, j, blQ, nC); #c1(RIMS1) c2(NP_001161880) c3(regulating synaptic membrane exocytosis protein 1 isoform 3) c4(66283) c5(66284, 66285, 66286, 66287, 66288, 66289) c6( ) c7(IR, nb, W, nx, aVu, j, blQ, nC); #c1(RIMS1) c2(NP_001161881) c3(regulating synaptic membrane exocytosis protein 1 isoform 4) c4(66290) c5( ) c6( ) c7(IR, nb, W, nx, aVu, j, blQ, nC); #c1(RIMS1) c2(NP_001161882) c3(regulating synaptic membrane exocytosis protein 1 isoform 5 precursor) c4(66291) c5(66292, 66293, 66294, 66295, 66296, 66297, 66298) c6( ) c7(IR, nb, W, nx, aVu, j, blQ, nC); #c1(RIMS1) c2(NP_001161883) c3(regulating synaptic membrane exocytosis protein 1 isoform 6) c4( ) c5(66299, 66300, 66301, 66302, 66303) c6( ) c7(IR, nb, W, nx, aVu, j, blQ, nC); #c1(RIMS1) c2(NP_055804) c3(regulating synaptic membrane exocytosis protein 1 isoform 1) c4(183598) c5(183599, 183600, 183601, 183602, 183603, 183604) c6( ) c7(IR, nb, W, nx, aVu, j, bll, nC); #c1(RIMS1) c2(XP_005248745) c3(regulating synaptic membrane exocytosis protein 1 isoform X7) c4(268519, 268520, 268521, 268522, 268523, 268524) c5(268525, 268526, 268527, 268528, 268529, 268530, 268531, 268532, 268533, 268534, 268535, 268536, 268537, 268538, 268539, 268540, 268541, 268542, 268543, 268544, 268545, 268546, 268547, 268548, 268549, 268550, 268551, 268552, 268553, 268554, 268555, 268556, 268557, 268558, 268559, 268560) c6( ) c7(IR, nb, W, nx, aVu, j, blQ, nC); #c1(RIMS1) c2(XP_011533909) c3(regulating synaptic membrane exocytosis protein 1 isoform X6) c4(330104) c5(330105, 330106, 330107, 330108, 330109, 330110) c6( ) c7(IR, nb, W, nx, aVu, j, blQ, nC); #c1(RIMS1) c2(XP_011533914) c3(regulating synaptic membrane exocytosis protein 1 isoform X12) c4( ) c5(330111, 330112, 330113, 330114, 330115) c6( ) c7(IR, nb, W, nx, aVu, j, bll, nC); #c1(RIMS1) c2(XP_011533920) c3(regulating synaptic membrane exocytosis protein 1 isoform X17) c4(330116) c5(330117) c6( ) c7(IR, nb, W, nx, aVu, j, blQ, nC); #c1(RIMS2) c2(NP_001093587) c3(regulating synaptic membrane exocytosis protein 2 isoform a) c4(40311) c5(40312, 40313, 40314, 40315, 40316, 40317) c6( ) c7(T, os, aC, bB, ci, x, aU, K, hu, H, eL, bc, aW, La, as, eE, cm, aAW, eM, nB, iT, vA, U, dW, nw, k, m, avU, NK, gU, Ol, ht, NT, n, aj, nA, qN, eW, ft, hw, Ak, h, aYf, dL, cC, oS, kj, aq, op, bP, mg, hR, bfX, fl, jd, it, B, L, bL, fM, dA, cjn, ew, ON, C, q, J, qH, er, Kx, MR, WY, aT, j, ae, bUd, lE, wr, iU, Y, r, aaE, lo, bu, aaO, t, ck, rR, alC, c, gX, bt, kh, pY, jh, oZ, dG, hF, Fc, mC, ad, Ae, oj, bj, ji, lQ, fb, nJ, uX, aCt, xs, eZ, app, KM, bz, bE, afd, lF, Kj, bC, lC, kS, iu, y); #c1(RIMS2) c2(NP_001269810) c3(regulating synaptic membrane exocytosis protein 2 isoform c) c4(102880) c5(102881, 102882, 102883, 102884, 102885, 102886) c6( ) c7(T, os, aC, bB, ci, x, aU, K, hu, H, eL, bc, aW, La, as, eE, cm, aAW, eM, nB, iT, vA, U, dW, nw, k, m, avU, NK, gU, Ol, ht, NT, n, aj, nA, qN, eW, ft, hw, Ak, h, aYf, dL, cC, oS, kj, aq, op, bP, mg, hR, bfX, fL, jd, it, B, L, bL, fM, dA, cjn, ew, ON, C, q, J, qH, er, Kx, MR, WY, aT, j, ae, bUd, lE, wr, iU, Y, r, aaE, lo, bu, aaO, t, ck, rR, alC, c, gX, bt, kh, pY, jh, oZ, dG, hF, Fc, mC, ad, Ae, oj, bj, ji, lQ, fb, nJ, uX, aCt, xs, eZ, app, KM, bz, bE, afd, lF, Kj, bC, lC, kS, iu, y); #c1(RIMS2) c2(NP_001269811) c3(regulating synaptic membrane exocytosis protein 2 isoform d) c4(102887) c5(102888, 102889, 102890, 102891, 102892, 102893) c6( ) c7(T, os, aC, bB, ci, x, aU, K, hu, H, eL, bc, aW, La, as, eE, cm, aAW, eM, nB, iT, vA, U, dW, nw, k, m, avl, NK, gU, Ol, ht, NT, n, aj, nA, qN, eW, ft, hw, Ak, h, aYf, dL, cC, oS, kj, aq, op, bP, mg, hR, bfX, fL, jd, it, B, L, bL, fM, dA, cjn, ew, ON, C, q, J, qH, er, Kx, MR, WY, aT, j, ae, bUd, lE, wr, iU, Y, r, aaE, lo, bu, aal, t, ck, rR, alC, c, gX, bt, kh, pY, jh, oZ, dG, hF, Fc, mC, ad, Ae, oj, bj, ji, lQ, fb, nJ, uX, aCt, xs, eZ, app, KM, bz, bE, afd, lF, Kj, bC, lC, KS, iu, y); #c1(RIMS2) c2(NP_055492) c3(regulating synaptic membrane exocytosis protein 2 isoform b) c4(181486) c5(181487, 181488, 181489, 181490, 181491, 181492, 181493, 181494, 181495, 181496, 181497, 181498) c6( ) c7(T, os, aC, bB, ci, x, aU, K, hu, H, eL, bc, aW, La, as, eE, cm, aAW, eM, nB, iT, vA, U, dW, nw, k, m, avU, NK, gU, Ol, ht, NT, n, aj, nA, qN, eW, ft, hw, Ak, h, aYf, dL, cC, oS, kj, aq, op, bP, mg, hR, bfX, fL, jd, it, B, L, bL, fM, dA, cjn, ew, ON, C, q, J, qH, er, Kx, MR, WY, aT, j, ae, bUd, lE, wr, iU, Y, r, aaE, lo, bu, aaO, t, ck, rR, alC, c, gX, bt, kh, pY, jh, oZ, dG, hF, Fc, mC, ad, Ae, oj, bj, ji, lQ, fb, nJ, uX, aCt, xs, eZ, app, KM, bz, bE, afd, lF, Kj, bC, lC, kS, iu, y); #c1(RIMS2) c2(XP_011515692) c3(regulating synaptic membrane exocytosis protein 2 isoform X2) c4(308532) c5( ) c6( ) c7(T, os, aC, bB, ci, x, aU, K, hu, H, eL, bc, aW, La, as, eE, cm, aAW, eM, nB, iT, vA, U, dW, nw, k, m, avU, NK, gU, Ol, ht, NT, n, aj, nA, qN, eW, ft, hw, Ak, h, aYf, dL, cC, oS, kj, aq, op, bP, mg, hR, bfX, fL, jd, it, B, L, bL, fM, dA, cjn, ew, ON, C, q, J, qH, er, Kx, MR, WY, aT, j, ae, bUd, lE, wr, iU, Y, r, aaE, lo, bu, aaO, t, ck, rR, alC, c, gX, bt, kh, pY, jh, oZ, dG, hF, Fc, mC, ad, Ae, oj, bj, ji, lQ, fb, nJ, uX, aCt, xs, eZ, app, KM, bz, bE, afd, lF, Kj, bC, lC, KS, iu, y); #c1(RIMS2) c2(XP_011515700) c3(regulating synaptic membrane exocytosis protein 2 isoform X13) c4(308533, 308534) c5(308535, 308536, 308537, 308538, 308539, 308540, 308541, 308542, 308543, 308544, 308545, 308546) c6( ) c7(T, os, aC, bB, ci, x, aU, K, hu, H, eL, bc, aW, La, as, eE, cm, aAW, eM, nB, iT, vA, U, dW, nw, k, m, avU, NK, gU, OI, ht, NT, n, aj, nA, qN, eW, ft, hw, Ak, h, aYf, dL, cC, oS, kj, aq, op, bP, mg, hR, bfX, fL, jd, it, B, L, bL, fM, dA, cjn, ew, ON, C, q, J, qH, er, Kx, MR, WY, aT, j, ae, bUd, lE, wr, iU, Y, r, aaE, lo, bu, aaO, t, ck, rR, alC, c, gX, bt, kh, pY, jh, oZ, dG, hF, Fc, mC, ad, Ae, oj, bj, ji, lQ, fb, nJ, uX, aCt, xs, eZ, app, KM, bz, bE, afd, lF, Kj, bC, lC, kS, iu, y); #c1(RIMS4) c2(NP_001192246) c3(regulating synaptic membrane exocytosis protein 4 isoform 1) c4(82142) c5(82143, 82144, 82145, 82146, 82147, 82148) c6( ) c7(are); #c1(RIMS4) c2(NP_892015) c3(regulating synaptic membrane exocytosis protein 4 isoform 2) c4(257611) c5(257612, 257613, 257614, 257615, 257616, 257617) c6( ) c7(are); #c1(RINI) c2(NP_004283) c3(ras and Rab interactor 1) c4(144827, 144828) c5(144829, 144830, 144831, 144832, 144833, 144834) c6( ) c7(iu, T, Jl, aW, Y, bE, ff, aT, bB, n, j, en, g, B, er); #c1(RINI) c2(XP_011543703) c3(ras and Rab interactor 1 isoform X3) c4( ) c5(342778, 342779, 342780, 342781, 342782, 342783) c6( ) c7(iu, T, JI, aW, Y, bE, ff, aT, bB, n, j, en, g, B, er); #c1(RIN2) c2(NP_001229510) c3(ras and Rab interactor 2 isoform 1) c4(84582) c5(84583, 84584, 84585, 84586, 84587, 84588) c6( ) c7(ac, czA, aT, h, aq, eA, ei, Xu, iR, bw, nJ, B, lU); #c1(RIN2) c2(XP_005260790) c3(ras and Rab interactor 2 isoform X7) c4( ) c5(276878, 276879, 276880, 276881, 276882, 276883) c6( ) c7(ac, czA, aT, h, aq, eA, ei, Xu, iR, bw, nJ, B, IU); #c1(RIN2) c2(XP_011527558) c3(ras and Rab interactor 2 isoform X3) c4(322402) c5( ) c6( ) c7(ac, czA, aT, h, aq, eA, ei, Xu, iR, bw, nJ, B, lU); #c1(RIN3) c2(NP_079108) c3(ras and Rab interactor 3) c4(217061, 217062) c5(217063, 217064, 217065, 217066, 217067, 217068, 217069) c6( ) c7(bi, bdM); #c1(RIN3) c2(XP_011535467) c3(ras and Rab interactor 3 isoform X3) c4(332528) c5(332529, 332530, 332531, 332532, 332533, 332534, 332535, 332536, 332537, 332538, 332539, 332540) c6( ) c7(bi, bdM); #c1(RING1) c2(NP_002922) c3(E3 ubiquitin-protein ligase RING1) c4(133584) c5(133585, 133586, 133587, 133588, 133589, 133590, 133591, 133592, 133593, 133594, 133595, 133596, 133597, 133598, 133599, 133600, 133601, 133602, 133603, 133604, 133605, 133606, 133607, 133608) c6( ) c7(ID, ly, es, A, BN, aE, iW, eZ, n, hy, vc, kp, fr, cM, ae, ne, ad, lx); #c1(RINT1) c2(NP_068749) c3(RAD50-interacting protein 1) c4(210170) c5(210171, 210172, 210173, 210174, 210175, 210176) c6( ) c7(aW, T, BO, aT); #c1(RINT1) c2(XP_011514760) c3(RAD50-interacting protein 1 isoform X1) c4( ) c5(307405) c6( ) c7(aW, T, BO, aT); #c1(RIOK1) c2(NP_113668) c3(serine/threonine-protein kinase RIO1 isoform 1) c4(222033) c5(222034, 222035, 222036, 222037, 222038, 222039) c6( ) c7(aC, d); #c1(RIOK2) c2(NP_001153221) c3(serine/threonine-protein kinase RIO2 isoform 2) c4(60551) c5(60552, 60553, 60554, 60555, 60556, 60557) c6( ) c7(uC, jv, d, cz); #c1(RIOK2) c2(NP_060813) c3(serine/threonine-protein kinase RIO2 isoform 1) c4(197954) c5(197955, 197956, 197957, 197958, 197959, 197960) c6( ) c7(uC, jv, d, cz); #c1(RIOK3) c2(NP_003822) c3(serine/threonine-protein kinase RIO3) c4(141170) c5(141171, 141172, 141173, 141174, 141175, 141176) c6( ) c7(aC, eJ); #c1(RIPK1) c2(NP_003795) c3(receptor-interacting serine/threonine-protein kinase 1) c4(140926, 140927) c5(140928, 140929, 140930, 140931, 140932, 140933, 140934, 140935, 140936, 140937, 140938, 140939, 140940, 140941, 140942, 140943, 140944) c6( ) c7(T, fM, dA, fR, iW, yG, czB, aj, ci, EA, q, J, ad, av, aE, ds, nl, aT, K, eG, d, aW, r, t, ik, lt, eM, bo, m, hE, bt, y); #c1(RIPK1) c2(XP_011513271) c3(receptor-interacting serine/threonine-protein kinase 1 isoform X2) c4(305545) c5( ) c6( ) c7(T, fM, dA, FR, iW, yG, czB, aj, ci, EA, q, J, ad, av, aE, ds, nl, aT, K, eG, d, aW, r, t, ik, lt, eM, bo, m, hE, bt, y); #c1(RIPK2) c2(NP_003812) c3(receptor-interacting serine/threonine-protein kinase 2) c4(141060) c5(141061, 141062, 141063, 141064, 141065, 141066, 141067) c6( ) c7(oO, bL, cZ, T, fM, dA, fR, ya, dD, bM, ea, e, jh, aA, ti, ww, eq, ci, q, ad, eA, bv, AW, l, hd, aE, u, hW, av, Dq, mj, Ou, gl, ji, dd, fb, ae, dx, li, tb, act, aCs, r, eZ, fe, af, zC, g, as, iV, FX, acD, cm, hq, rR, acC, eM, dB, gX, On); #c1(RIPK2) c2(XP_005251149) c3(receptor-interacting serine/threonine-protein kinase 2 isoform X1) c4(270839) c5(270840, 270841, 270842, 270843, 270844) c6( ) c7(oO, bL, cZ, T, fM, dA, fR, ya, dD, bM, ea, e, jh, aA, ti, ww, eq, ci, q, ad, eA, bv, AW, l, hd, aE, u, hW, av, Dq, mj, Ou, gl, ji, dd, fb, ae, dx, li, tb, aCt, aCs, r, eZ, fe, aF, zC, g, as, iV, FX, acD, cm, hq, rR, acC, eM, dB, gX, On); #c1(RIPK3) c2(NP_006862) c3(receptor-interacting serine/threonine-protein kinase 3) c4(168695) c5(168696, 168697, 168698, 168699, 168700, 168701) c6( ) c7(av, ft, d, dD, up, ea, ds, AQ, iW, nl, ceC, cj, fe, nw, v, K, lS, eq, wb, hE); #c1(RIPK4) c2(NP_065690) c3(receptor-interacting serine/threonine-protein kinase 4) c4(204939, 204940, 204941, 204942) c5(204943, 204944, 204945, 204946, 204947, 204948, 204949, 204950, 204951, 204952, 204953, 204954, 204955, 204956, 204957, 204958, 204959, 204960, 204961, 204962, 204963, 204964, 204965, 204966, 204967, 204968, 204969, 204970) c6( ) c7(fM, czC, czB, j, f, Vj, bTm); #c1(RIPPLY2) c2(NP_001009994) c3(protein ripply2) c4(23801, 23802) c5(23803, 23804, 23805, 23806, 23807, 23808, 23809, 23810, 23811, 23812, 23813, 23814) c6( ) c7(XW); #c1(RIT1) c2(NP_001243749) c3(GTP-binding protein Rit1 isoform 3) c4( ) c5(91641, 91642, 91643, 91644, 91645) c6( ) c7(ck, T, iR, yS, Az, czD, bD, aq, Gp, B); #c1(RIT1) c2(NP_001243750) c3(GTP-binding protein Rit1 isoform 1) c4(91646) c5(91647, 91648, 91649, 91650, 91651, 91652, 91653) c6( ) c7(ck, T, iR, yS, Az, czD, bD, aq, Gp, B); #c1(RIT1) c2(NP_008843) c3(GTP-binding protein Rit1 isoform 2) c4(168987, 168988) c5(168989, 168990, 168991, 168992, 168993, 168994) c6( ) c7(ck, T, iR, yS, Az, czD, bD, aq, Gp, B); #c1(RIT2) c2(NP_001259006) c3(GTP-binding protein Rit2 isoform 2) c4(96932) c5(96933, 96934, 96935, 96936, 96937, 96938) c6( ) c7(qC, bL, mj, F, iA, jx, hl, n, cy, v, fr, apt, an, Gp, hE, fq, y, vi); #c1(RIT2) c2(NP_002921) c3(GTP-binding protein Rit2 isoform 1) c4(133577) c5(133578, 133579, 133580, 133581, 133582, 133583) c6( ) c7(qC, bL, mj, F, iA, jx, hl, n, cy, v, fr, apt, an, Gp, hE, fq, y, vi); #c1(RITA1) c2(NP_001273144) c3(RBPJ-interacting and tubulin-associated protein 1 isoform 2) c4(104367, 104368) c5(104369, 104370, 104371, 104372, 104373, 104374, 104375, 104376, 104377, 104378) c6( ) c7(ck, T, nB, aW, cj, aT, fd, cm, bt, eJ, dx); #c1(RITA1) c2(NP_116237) c3(RBPJ-interacting and tubulin-associated protein 1 isoform 1) c4(225885) c5(225886, 225887, 225888, 225889, 225890, 225891, 225892, 225893, 225894, 225895, 225896, 225897, 225898, 225899, 225900, 225901) c6( ) c7(ck, T, nB, aW, cj, aT, fd, cm, bt, eJ, dx); #c1(RLBP1) c2(XP_011520172) c3(retinaldehyde-binding protein 1 isoform X1) c4(315041) c5(315042, 315043, 315044, 315045, 315046, 315047) c6( ) c7(bF, apo, lR, bnP, nx, aSW, nw, czF, OL, nk, czE, db, qW, cap, nC); #c1(RLF) c2(XP_011540196) c3(zinc finger protein RIf isoform X2) c4( ) c5(338603) c6( ) c7(ft, Kw); #c1(RLIM) c2(NP_899196) c3(E3 ubiquitin-protein ligase RLIM) c4(258000) c5(258001, 258002, 258003, 258004, 258005, 258006, 258007, 258008, 258009, 258010, 258011) c6( ) c7(ad); #c1(RLN1) c2(NP_008842) c3(prorelaxin H1 preproprotein) c4(168979) c5(168980, 168981, 168982, 168983, 168984, 168985, 168986) c6( ) c7(T, nB, vT, aW, Lf, cz, aT, ad); #c1(RLN2) c2(NP_005050) c3 (prorelaxin H2 isoform 2 precursor) c4(152448, 152449) c5(152450, 152451, 152452, 152453, 152454, 152455, 152456, 152457, 152458, 152459) c6( ) c7(T, hd, nB, aW, aw, aT, K, ad); #c1(RLN2) c2(NP_604390) c3(prorelaxin H2 isoform 1 preproprotein) c4(235775) c5(235776, 235777, 235778, 235779, 235780, 235781, 235782, 235783, 235784, 235785, 235786, 235787, 235788, 235789, 235790, 235791, 235792, 235793, 235794, 235795, 235796, 235797, 235798, 235799, 235800, 235801, 235802, 235803, 235804, 235805, 235806, 235807, 235808, 235809, 235810, 235811, 235812, 235813, 235814, 235815, 235816, 235817, 235818, 235819, 235820, 235821, 235822, 235823, 235824, 235825, 235826, 235827, 235828, 235829, 235830, 235831, 235832, 235833, 235834, 235835, 235836, 235837, 235838, 235839, 235840, 235841, 235842, 235843, 235844, 235845, 235846, 235847, 235848, 235849, 235850, 235851, 235852, 235853, 235854, 235855, 235856, 235857, 235858, 235859, 235860, 235861, 235862, 235863, 235864, 235865, 235866, 235867, 235868, 235869) c6(235870) c7(T, hd, nB, aW, aw, aT, K, ad); #c1(RLN3) c2(NP_543140) c3(relaxin-3 preproprotein) c4(233380) c5(233381, 233382, 233383, 233384, 233385, 233386) c6( ) c7(j, K); #c1(RMDN1) c2(NP_001273648) c3(regulator of microtubule dynamics protein 1 isoform 3) c4(105543) c5(105544, 105545, 105546, 105547, 105548, 105549) c6( ) c7(T, hl, fM, GC, C, cd, q, ad, Hx, cbH, aE, h, cy, K, XZ, ae, af, d, aW, aml, Y, ff, r, en, U, m, B); #c1(RMDN1) c2(NP_057117) c3(regulator of microtubule dynamics protein 1 isoform 1) c4(188980) c5(188981, 188982, 188983, 188984, 188985, 188986) c6( ) c7(T, hl, fM, GC, C, cd, q, ad, Hx, cbH, aE, h, cy, K, XZ, ae, af, d, aW, aml, Y, ff, r, en, U, m, B); #c1(RMDN2) c2(NP_001164263) c3(regulator of microtubule dynamics protein 2 isoform 2) c4(66855, 66856) c5(66857, 66858, 66859, 66860, 66861, 66862, 66863, 66864, 66865, 66866, 66867, 66868, 66869, 66870, 66871, 66872) c6( ) c7(hl, jh, GC, C, cd, ad, x, Hx, cbH, aE, h, cy, dL, K, XZ, ae, af, d, aW, aml, Y, ff, en, W, U, at, k, m, B); #c1(RMDN2) c2(NP_001164264) c3(regulator of microtubule dynamics protein 2 isoform 3) c4(66873, 66874) c5(66875, 66876, 66877, 66878, 66879, 66880, 66881, 66882, 66883, 66884, 66885, 66886, 66887, 66888, 66889, 66890, 66891, 66892) c6( ) c7(hl, jh, GC, C, cd, ad, x, Hx, cbH, aE, h, cy, dL, K, XZ, ae, af, d, aW, aml, Y, ff, en, W, U, at, k, m, B); #c1(RMDN2) c2(NP_653314) c3(regulator of microtubule dynamics protein 2 isoform 1) c4( ) c5(239559, 239560, 239561, 239562, 239563, 239564) c6( ) c7(hl, jh, GC, C, cd, ad, x, Hx, cbH, aE, h, cy, dL, K, XZ, ae, af, d, aW, aml, Y, ff, en, W, U, at, k, m, B); #c1(RMDN2) c2(XP_011530916) c3(regulator of microtubule dynamics protein 2 isoform X1) c4( ) c5(326913) c6( ) c7(hl, jh, GC, C, cd, ad, x, Hx, cbH, aE, h, cy, dL, K, XZ, ae, af, d, aW, aml, Y, ff, en, W, U, at, k, m, B); #c1(RMDN2) c2(XP_011530917) c3(regulator of microtubule dynamics protein 2 isoform X2) c4(326914, 326915) c5( ) c6( ) c7(hl, jh, GC, C, cd, ad, x, Hx, cbH, aE, h, cy, dL, K, XZ, ae, af, d, aW, aml, Y, ff, en, W, U, at, k, m, B); #c1(RMDN3) c2(NP_001291731) c3(regulator of microtubule dynamics protein 3) c4(116434, 116435, 116436) c5(116437, 116438, 116439, 116440, 116441, 116442, 116443, 116444, 116445, 116446, 116447, 116448, 116449, 116450, 116451, 116452, 116453, 116454, 116455, 116456, 116457, 116458) c6( ) c7(T, hl, GC, ci, C, cd, ad, eA, Hx, afr, cbH, aE, h, cy, K, XZ, ae, af, d, aW, aml, Y, ff, en, sS, U, m, bt, B); #c1(RM11) c2(XP_011517336) c3(recQ-mediated genome instability protein 1 isoform X1) c4(311004, 311005) c5(311006, 311007, 311008, 311009, 311010, 311011, 311012, 311013, 311014, 311015, 311016, 311017) c6( ) c7(T, ac, hk, Y, gK, ew, r, bw, bt, q, br, ad); #c1(RM12) c2(NP_689521) c3(recQ-mediated genome instability protein 2) c4(242970, 242971) c5(242972, 242973, 242974, 242975, 242976, 242977, 242978, 242979, 242980, 242981, 242982, 242983) c6( ) c7(aj, Bv, j, K, A); #c1(RMND1) c2(NP_001258866) c3(required for meiotic nuclear division protein 1 homolog isoform 2) c4( ) c5(96605, 96606, 96607, 96608, 96609, 96610, 96611) c6( ) c7(hb, cc, czG, fM); #c1(RMND1) c2(NP_060379) c3(required for meiotic nuclear division protein 1 homolog isoform 1) c4(195702, 195703, 195704, 195705) c5(195706, 195707, 195708, 195709, 195710, 195711) c6( ) c7(hb, cc, czG, fM); #c1(RMND1) c2(XP_005267097) c3(required for meiotic nuclear division protein 1 homolog isoform X1) c4( ) c5(280680, 280681, 280682, 280683, 280684, 280685, 280686, 280687, 280688, 280689, 280690) c6( ) c7(hb, cc, czG, fM); #c1(RNASE1) c2(NP_937875) c3(ribonuclease pancreatic precursor) c4(259362) c5(259363, 259364, 259365, 259366, 259367, 259368, 259369, 259370, 259371, 259372, 259373, 259374, 259375, 259376, 259377, 259378, 259379, 259380) c6( ) c7(T, aC, pf, azi, if, bB, bv, aE, K, aCU, LC, aW, Ls, hl, as, cm, Qt, eM, iT, TG, U, cj, n, BF, ct, nA, qN, fr, no, ep, kH, h, By, wb, Um, dx, xf, fe, kp, yq, bQL, YE, dB, PI, ajW, B, fG, aA, cz, q, eA, lg, aT, cy, dz, zp, ae, jf, afG, Y, r, en, t, bN, ck, ac, c, AH, HK, mf, dy, aps, zu, bt, jh, bS, zU, iJ, ad, fU, ds, vw, ji, NP, d, BO, ff, aF, iV, Z, bz, dr, ek, Ew, yE, bE, X, aS, iu); #c1(RNASE11) c2(NP_660293) c3(probable ribonuclease Il precursor) c4(240669, 240670, 240671, 240672, 240673) c5(240674, 240675, 240676, 240677, 240678, 240679, 240680, 240681, 240682, 240683, 240684, 240685, 240686, 240687, 240688, 240689, 240690, 240691, 240692, 240693, 240694, 240695, 240696, 240697, 240698, 240699, 240700, 240701, 240702, 240703, 240704, 240705, 240706, 240707, 240708, 240709, 240710, 240711, 240712, 240713, 240714, 240715, 240716, 240717, 240718, 240719, 240720, 240721, 240722, 240723, 240724, 240725, 240726, 240727, 240728, 240729, 240730, 240731, 240732, 240733, 240734, 240735, 240736, 240737, 240738, 240739, 240740, 240741, 240742, 240743, 240744, 240745, 240746, 240747, 240748, 240749) c6(240750, 240751, 240752, 240753, 240754, 240755, 240756, 240757, 240758, 240759, 240760, 240761, 240762, 240763, 240764, 240765, 240766, 240767, 240768, 240769) c7(aVk); #c1(RNASE12) c2(NP_001019993) c3(probable inactive ribonuclease-like protein 12 precursor) c4(28000) c5(28001, 28002, 28003, 28004, 28005, 28006, 28007, 28008, 28009, 28010, 28011, 28012, 28013, 28014) c6(28015, 28016, 28017, 28018) c7(bN, cH, om, cj, v, nA, bD, biW, all); #c1(RNASE13) c2(NP_001012264) c3(probable inactive ribonuclease-like protein 13 precursor) c4(24814) c5(24815, 24816, 24817, 24818, 24819, 24820, 24821, 24822) c6(24823, 24824) c7(azx, VM); #c1(RNASE2) c2(NP_002925) c3(non-secretory ribonuclease precursor) c4(133609) c5(133610, 133611, 133612, 133613, 133614, 133615) c6( ) c7(bM, cT, aDu, tW, ac, z, oy, aXb, akC, bBH, uu, ey, eE, t, qa); #c1(RNASE3) c2(NP_002926) c3(eosinophil cationic protein precursor) c4(133616, 133617) c5(133618, 133619, 133620, 133621, 133622, 133623) c6( ) c7(bM, T, JN, aDy, aoj, Jx, nE, eh, baA, yt, oW, aZW, cU, dV, bYH, aDp, Cy, Nr, czH, sp, bfz, ff, po, lo, bBH, en, aFd, eE, zV, Nq, JA, z, aDB, JM, aNn, ne); #c1(RNASE4) c2(NP_002928) c3(ribonuclease 4 precursor) c4(133631) c5(133632, 133633, 133634, 133635, 133636, 133637, 133638, 133639, 133640, 133641, 133642, 133643, 133644, 133645, 133646, 133647, 133648, 133649, 133650, 133651, 133652, 133653, 133654, 133655, 133656, 133657, 133658, 133659, 133660, 133661, 133662, 133663) c6(133664, 133665, 133666, 133667, 133668, 133669) c7(fQ, aVk, kf, v, ad); #c1(RNASE6) c2(NP_005606) c3(ribonuclease K6 precursor) c4(157904) c5(157905, 157906, 157907, 157908, 157909, 157910) c6( ) c7(bL, hl, aj, ey, nE, J, iq); #c1(RNASE7) c2(NP_115961) c3(ribonuclease 7 precursor) c4(224943, 224944) c5(224945, 224946, 224947, 224948, 224949, 224950, 224951, 224952, 224953, 224954, 224955, 224956, 224957, 224958) c6(224959, 224960, 224961, 224962) c7(nE, eE, uF); #c1(RNASE8) c2(NP_612204) c3(ribonuclease 8 precursor) c4(236203) c5(236204, 236205, 236206, 236207, 236208, 236209) c6( ) c7(aVk); #c1(RNASE9) c2(NP_001103826) c3(inactive ribonuclease-like protein 9 isoform 2 precursor) c4( ) c5(41963, 41964, 41965, 41966, 41967, 41968, 41969, 41970, 41971, 41972, 41973, 41974, 41975, 41976, 41977, 41978, 41979, 41980, 41981, 41982, 41983, 41984, 41985, 41986, 41987, 41988, 41989, 41990, 41991, 41992, 41993, 41994) c6(41995, 41996, 41997, 41998, 41999, 42000, 42001, 42002, 42003, 42004, 42005, 42006) c7(ad); #c1(RNASE9) c2(NP_001103829) c3(inactive ribonuclease-like protein 9 isoform 1 precursor) c4(42007, 42008) c5(42009, 42010, 42011, 42012, 42013, 42014, 42015, 42016, 42017, 42018, 42019, 42020, 42021, 42022, 42023, 42024, 42025, 42026, 42027, 42028, 42029, 42030, 42031, 42032, 42033, 42034, 42035, 42036, 42037, 42038, 42039, 42040) c6(42041, 42042, 42043, 42044, 42045, 42046, 42047, 42048) c7(ad); #c1(RNASEH1) c2(NP_002927) c3(ribonuclease H1 isoform 1 precursor) c4(133624) c5(133625, 133626, 133627, 133628, 133629, 133630) c6( ) c7(T, kF, aF, aA); #c1(RNASEH2A) c2(NP_006388) c3(ribonuclease H2 subunit A) c4(164578) c5(164579, 164580, 164581, 164582, 164583, 164584) c6( ) c7(T, Ez, kh, iU, et, czl, cr, dz, pm, cl, hE, B, Ex); #c1(RNASEH2B) c2(NP_001135751) c3(ribonuclease H2 subunit B isoform 2) c4(53074) c5(53075, 53076, 53077, 53078, 53079, 53080) c6( ) c7(aSu, czJ, Ex, FR); #c1(RNASEH2B) c2(NP_078846) c3(ribonuclease H2 subunit B isoform 1) c4(215682, 215683) c5(215684, 215685, 215686, 215687, 215688, 215689, 215690, 215691, 215692, 215693, 215694, 215695) c6( ) c7(aSu, czJ, Ex, fR); #c1(RNASEH2C) c2(NP_115569) c3(ribonuclease H2 subunit C) c4(223236) c5(223237, 223238, 223239, 223240, 223241, 223242) c6( ) c7(wR, Nj, czK, Ex, wB); #c1(RNASEK) c2(NP_001004333) c3(ribonuclease kappa) c4(20591, 20592) c5(20593, 20594, 20595, 20596, 20597, 20598, 20599, 20600, 20601, 20602, 20603, 20604) c6( ) c7(t, T, m); #c1(RNASEL) c2(NP_066956) c3(2-5A-dependent ribonuclease) c4(207731) c5(207732, 207733, 207734, 207735, 207736, 207737, 207738, 207739, 207740, 207741) c6( ) c7(T, aC, fR, if, iW, jx, bB, OH, ci, qN, Do, acn, wy, q, ad, F, aE, aT, io, dL, hB, is, ae, af, cgd, BO, Ub, iU, aW, ff, r, nY, aq, lo, en, t, bz, AH, aiM, at, zv, adg, hE, B); #c1(RNASEL) c2(XP_005245468) c3(2-5A-dependent ribonuclease isoform X1) c4(266576, 266577) c5(266578, 266579, 266580, 266581, 266582, 266583, 266584, 266585, 266586, 266587, 266588, 266589) c6( ) c7(T, aC, fR, if, iW, jx, bB, oH, ci, qN, Do, acn, wy, q, ad, F, aE, aT, io, dL, hB, is, ae, af, cgd, BO, Ub, iU, aW, ff, r, nY, aq, lo, en, t, bz, AH, aiM, at, zv, adg, hE, B); #c1(RNASET2) c2(NP_003721) c3(ribonuclease T2 precursor) c4(140405, 140406, 140407, 140408, 140409) c5(140410, 140411, 140412, 140413, 140414, 140415, 140416, 140417, 140418, 140419, 140420, 140421, 140422, 140423, 140424, 140425, 140426, 140427, 140428, 140429, 140430, 140431, 140432, 140433, 140434, 140435, 140436, 140437, 140438, 140439, 140440, 140441, 140442, 140443) c6( ) c7(T, hb, Y, Ju, HD, czL, aVk, qj, K, m, t, pC); #c1(RND1) c2(NP_055285) c3(rho-related GTP-binding protein Rho6 precursor) c4(180282, 180283) c5(180284, 180285, 180286, 180287, 180288, 180289, 180290, 180291, 180292, 180293, 180294, 180295) c6( ) c7(aA, aT); #c1(RND3) c2(NP_005159) c3(rho-related GTP-binding protein RhoE precursor) c4(153491, 153492, 153493) c5(153494, 153495, 153496, 153497, 153498, 153499, 153500, 153501, 153502, 153503, 153504, 153505, 153506, 153507, 153508, 153509, 153510, 153511, 153512, 153513, 153514, 153515, 153516, 153517, 153518, 153519) c6( ) c7(bL, ae, T, cc, Of, s, ac, aW, Y, oe, ff, aA, cd, aF, K, en, fq, B, ad); #c1(RNF103) c2(NP_001185880) c3(E3 ubiquitin-protein ligase RNF103 isoform 2) c4( ) c5(76629, 76630, 76631, 76632, 76633, 76634) c6( ) c7(ct); #c1(RNF103) c2(NP_005658) c3(E3 ubiquitin-protein ligase RNF103 isoform 1) c4(158332) c5( ) c6( ) c7(ct); #c1(RNF103-CHMP3) c2(NP_001185883) c3(RNF103-CHMP3 protein) c4(76642, 76643) c5(76644, 76645, 76646, 76647, 76648, 76649) c6( ) c7(h, f, ae, ad); #c1(RNF111) c2(XP_006720638) c3(E3 ubiquitin-protein ligase Arkadia isoform X8) c4(293596) c5(293597, 293598, 293599, 293600, 293601) c6( ) c7(cd, mx, ll); #c1(RNF111) c2(XP_006720642) c3(E3 ubiquitin-protein ligase Arkadia isoform X5) c4( ) c5(293602, 293603, 293604, 293605, 293606, 293607, 293608, 293609, 293610) c6( ) c7(cd, mx, ll); #c1(RNF111) c2(XP_011519998) c3(E3 ubiquitin-protein ligase Arkadia isoform X2) c4(314855) c5(314856, 314857, 314858, 314859, 314860) c6( ) c7(cd, mx, ll); #c1(RNF111) c2(XP_011519999) c3(E3 ubiquitin-protein ligase Arkadia isoform X4) c4(314861) c5(314862, 314863, 314864, 314865, 314866, 314867) c6( ) c7(cd, mx, ll); #c1(RNF111) c2(XP_011520001) c3(E3 ubiquitin-protein ligase Arkadia isoform X1) c4(314868, 314869) c5(314870, 314871, 314872, 314873, 314874) c6( ) c7(cd, mx, ll); #c1(RNF111) c2(XP_011520002) c3(E3 ubiquitin-protein ligase Arkadia isoform X3) c4( ) c5(314875, 314876, 314877, 314878, 314879, 314880) c6( ) c7(cd, mx, ll); #c1(RNF112) c2(NP_009079) c3(RING finger protein 112) c4(170586) c5(170587, 170588, 170589, 170590, 170591, 170592, 170593) c6( ) c7(xy, W, czM, aGU, nA, aHj); #c1(RNF112) c2(XP_006721634) c3(RING finger protein 112 isoform X1) c4( ) c5(294138) c6( ) c7(xy, W, czM, aGU, nA, aHj); #c1(RNF114) c2(NP_061153) c3(E3 ubiquitin-protein ligase RNF114) c4(199600, 199601) c5(199602, 199603, 199604, 199605, 199606, 199607, 199608, 199609, 199610, 199611, 199612, 199613, 199614) c6( ) c7(wD, cr, an); #c1(RNF123) c2(NP_071347) c3(E3 ubiquitin-protein ligase RNF123) c4(211127, 211128) c5(211129, 211130, 211131, 211132, 211133, 211134, 211135, 211136, 211137, 211138, 211139, 211140) c6( ) c7(Z, bS, K, ct, bdq, bdr); #c1(RNF125) c2(NP_060301) c3(E3 ubiquitin-protein ligase RNF125) c4(195273) c5(195274, 195275, 195276, 195277, 195278, 195279) c6( ) c7(q, W, r); #c1(RNF128) c2(NP_078815) c3(E3 ubiquitin-protein ligase RNF128 isoform 2 precursor) c4(215608, 215609) c5(215610, 215611, 215612, 215613, 215614, 215615, 215616, 215617, 215618, 215619, 215620, 215621) c6( ) c7(Pp, iV); #c1(RNF128) c2(NP_919445) c3(E3 ubiquitin-protein ligase RNF128 isoform 1 precursor) c4(258635) c5(258636, 258637, 258638, 258639, 258640, 258641) c6( ) c7(Pp, iV); #c1(RNF130) c2(NP_060904) c3(E3 ubiquitin-protein ligase RNF130 isoform 1 precursor) c4(198566) c5(198567, 198568, 198569, 198570, 198571, 198572) c6( ) c7(j, ad); #c1(RNF135) c2(NP_001171921) c3(E3 ubiquitin-protein ligase RNF135 isoform 3) c4(72026, 72027) c5(72028, 72029, 72030, 72031, 72032, 72033, 72034, 72035, 72036, 72037, 72038) c6( ) c7(T, nX, Az, hP, xb, xt, czN); #c1(RNF135) c2(NP_115698) c3(E3 ubiquitin-protein ligase RNF135 isoform 1) c4(223607) c5(223608, 223609, 223610, 223611, 223612, 223613, 223614, 223615, 223616) c6( ) c7(T, nX, Az, hP, xb, xt, czN); #c1(RNF135) c2(NP_922921) c3(E3 ubiquitin-protein ligase RNF135 isoform 2) c4(258642, 258643) c5(258644, 258645, 258646, 258647, 258648, 258649, 258650, 258651, 258652, 258653) c6( ) c7(T, nX, Az, hP, xb, xt, czN); #c1(RNF135) c2(XP_005258100) c3(E3 ubiquitin-protein ligase RNF135 isoform X2) c4( ) c5(275487, 275488, 275489, 275490, 275491, 275492, 275493) c6( ) c7(T, nX, Az, hP, xb, xt, czN); #c1(RNF135) c2(XP_006722201) c3(E3 ubiquitin-protein ligase RNF135 isoform X1) c4(294634) c5( ) c6( ) c7(T, nX, Az, hP, xb, xt, czN); #c1(RNF138) c2(NP_057355) c3(E3 ubiquitin-protein ligase RNF138 isoform 1) c4(190313) c5(190314, 190315, 190316, 190317, 190318, 190319) c6( ) c7(ey); #c1(RNF138) c2(NP_937761) c3(E3 ubiquitin-protein ligase RNF138 isoform 2) c4(259061) c5(259062, 259063, 259064, 259065, 259066, 259067) c6( ) c7(ey); #c1(RNF139) c2(NP_009149) c3(E3 ubiquitin-protein ligase RNF139) c4(171147) c5(171148, 171149, 171150, 171151, 171152, (71153) c6( ) c7(es, czO, sS, T, d, hd, nB, nv, iA, py, go, fr, Le); #c1(RNF14) c2(XP_005268598) c3(E3 ubiquitin-protein ligase RNF14 isoform X2) c4(281937) c5(281938, 281939, 281940, 281941, 281942) c6( ) c7(T, c, bE, bB, vH, ci, pt, ae, ad); #c1(RNF14) c2(XP_005268600) c3(E3 ubiquitin-protein ligase RNF14 isoform X3) c4(281943, 281944, 281945) c5(281946, 281947, 281948, 281949, 281950, 281951, 281952, 281953, 281954, 281955, 281956, 281957, 281958, 281959, 281960, 281961, 281962, 281963, 281964, 281965, 281966, 281967, 281968, 281969) c6( ) c7(T, c, bE, bB, vH, ci, pt, ae, ad); #c1(RNF14) c2(XP_011536016) c3(E3 ubiquitin-protein ligase RNF14 isoform X1) c4(333277, 333278, 333279, 333280, 333281, 333282, 333283) c5(333284, 333285, 333286, 333287, 333288, 333289, 333290, 333291, 333292, 333293, 333294, 333295, 333296, 333297, 333298, 333299, 333300, 333301, 333302, 333303, 333304, 333305, 333306, 333307, 333308, 333309, 333310, 333311, 333312, 333313, 333314, 333315, 333316, 333317, 333318, 333319, 333320, 333321, 333322, 333323, 333324, 333325, 333326, 333327, 333328, 333329, 333330, 333331, 333332) c6( ) c7(T, c, bE, bB, vH, ci, pt, ae, ad); #c1(RNF144A) c2(XP_005246259) c3(E3 ubiquitin-protein ligase RNF144A isoform X1) c4( ) c5(266996, 266997, 266998, 266999, 267000) c6( ) c7(T, ac, c, aW, al, Ea); #c1(RNF144A) c2(XP_006711972) c3(E3 ubiquitin-protein ligase RNF144A isoform X2) c4(286310) c5(286311, 286312) c6( ) c7(T, ac, c, aW, aT, Ea); #c1(RNF144A) c2(XP_011508726) c3(E3 ubiquitin-protein ligase RNF144A isoform X3) c4(299348, 299349) c5(299350, 299351, 299352, 299353, 299354, 299355, 299356, 299357, 299358, 299359, 299360, 299361, 299362, 299363, 299364, 299365) c6( ) c7(T, ac, c, aW, aT, Ea); #c1(RNF144B) c2(XP_005249041) c3(E3 ubiquitin-protein ligase RNF144B isoform X1) c4(268937, 268938) c5(268939, 268940, 268941, 268942, 268943, 268944) c6( ) c7(oe, K); #c1(RNF144B) c2(XP_005249043) c3(E3 ubiquitin-protein ligase RNF144B isoform X2) c4( ) c5(268945, 268946, 268947, 268948, 268949, 268950) c6( ) c7(oe, K); #c1(RNF146) c2(NP_001229778) c3(E3 ubiquitin-protein ligase RNF146 isoform b) c4(85229) c5(85230, 85231, 85232, 85233, 85234, 85235) c6( ) c7(eM, aW, aT, t, bz, af); #c1(RNF149) c2(NP_775918) c3(E3 ubiquitin-protein ligase RNF149 precursor) c4(250676) c5(250677, 250678, 250679, 250680, 250681, 250682) c6( ) c7(aC); #c1(RNF150) c2(NP_065775) c3(RING finger protein 150 precursor) c4(205328, 205329, 205330) c5(205331, 205332, 205333, 205334, 205335, 205336, 205337, 205338, 205339, 205340, 205341, 205342, 205343, 205344, 205345, 205346, 205347, 205348) c6( ) c7(Gp); #c1(RNF150) c2(XP_005263207) c3(RING finger protein 150 isoform X1) c4(278358) c5( ) c6( ) c7(Gp); #c1(RNF150) c2(XP_011530449) c3(RING finger protein 150 isoform X2) c4( ) c5(326309) c6( ) c7(Gp); #c1(RNF150) c2(XP_011530450) c3(RING finger protein 150 isoform X3) c4( ) c5(326310, 326311, 326312, 326313, 326314, 326315, 326316, 326317, 326318) c6( ) c7(Gp); #c1(RNF152) c2(NP_775828) c3(E3 ubiquitin-protein ligase RNF152) c4(250453) c5(250454, 250455, 250456, 250457, 250458, 250459) c6( ) c7(Kw); #c1(RNF157) c2(NP_443148) c3(RING finger protein 157) c4(229633, 229634) c5(229635, 229636, 229637, 229638, 229639, 229640, 229641, 229642, 229643, 229644, 229645, 229646) c6( ) c7(oe); #c1(RNF157) c2(XP_005257064) c3(RING finger protein 157 isoform X2) c4(274653) c5( ) c6( ) c7(oe); #c1(RNF157) c2(XP_011522575) c3(RING finger protein 157 isoform X6) c4( ) c5(317167, 317168, 317169, 317170, 317171, 317172) c6( ) c7(oe); #c1(RNF167) c2(NP_056343) c3(E3 ubiquitin-protein ligase RNF167 precursor) c4(187165) c5(187166, 187167, 187168, 187169, 187170, 187171) c6( ) c7(czP); #c1(RNF168) c2(NP_689830) c3(E3 ubiquitin-protein ligase RNF168) c4(244072) c5(244073, 244074, 244075, 244076, 244077, 244078, 244079, 244080, 244081) c6( ) c7(aW, T, auV, aT); #c1(RNF170) c2(NP_001153695) c3(E3 ubiquitin-protein ligase RNF170 isoform a) c4(60855, 60856, 60857) c5(60858, 60859, 60860, 60861, 60862, 60863, 60864, 60865, 60866, 60867, 60868, 60869) c6( ) c7(czQ, bcT); #c1(RNF170) c2(NP_001153696) c3(E3 ubiquitin-protein ligase RNF170 isoform b) c4(60870, 60871) c5(60872, 60873, 60874, 60875, 60876, 60877) c6( ) c7(czQ, bcT); #c1(RNF170) c2(NP_001153697) c3(E3 ubiquitin-protein ligase RNF170 isoform c) c4( ) c5(60878, 60879, 60880, 60881, 60882) c6( ) c7(czQ, bcT); #c1(RNF170) c2(XP_006716468) c3(E3 ubiquitin-protein ligase RNF170 isoform X3) c4(290330) c5(290331, 290332, 290333, 290334, 290335) c6( ) c7(czQ, bcT); #c1(RNF170) c2(XP_006716470) c3(E3 ubiquitin-protein ligase RNF170 isoform X4) c4( ) c5(290336, 290337, 290338, 290339, 290340, 290341, 290342, 290343, 290344, 290345, 290346, 290347) c6( ) c7(czQ, bcT); #c1(RNF180) c2(NP_001107033) c3(E3 ubiquitin-protein ligase RNF180 isoform 1) c4(42799, 42800) c5(42801, 42802, 42803, 42804, 42805, 42806) c6( ) c7(T, aF, A, aA); #c1(RNF180) c2(NP_848627) c3(E3 ubiquitin-protein ligase RNF180 isoform 2) c4(254022, 254023) c5(254024, 254025, 254026, 254027, 254028, 254029) c6( ) c7(T, aF, A, aA); #c1(RNF180) c2(XP_011541645) c3(E3 ubiquitin-protein ligase RNF180 isoform X3) c4( ) c5(340186, 340187, 340188, 340189, 340190, 340191, 340192, 340193, 340194, 340195, 340196, 340197) c6( ) c7(T, aF, A, aA); #c1(RNF182) c2(NP_001158506) c3(E3 ubiquitin-protein ligase RNF182) c4(63633, 63634, 63635, 63636, 63637) c5(63638, 63639, 63640, 63641, 63642, 63643, 63644, 63645, 63646, 63647, 63648, 63649, 63650, 63651, 63652, 63653, 63654, 63655, 63656, 63657, 63658, 63659, 63660, 63661, 63662, 63663, 63664, 63665, 63666, 63667, 63668, 63669, 63670, 63671, 63672, 63673, 63674, 63675, 63676, 63677, 63678) c6( ) c7(y, aw, aT); #c1(RNF19A) c2(XP_005250910) c3(E3 ubiquitin-protein ligase RNF19A isoform X1) c4(270625, 270626) c5(270627, 270628, 270629, 270630, 270631, 270632, 270633, 270634, 270635, 270636, 270637, 270638, 270639, 270640) c6( ) c7(T, aC, ea, if, fd, bB, gf, lR, Lw, bv, xE, aE, rw, dV, K, aW, zC, g, as, eF, cm, bw, eM, cj, NO, Hb, m, NN, NK, dD, NJ, aj, BF, ct, qN, fr, cd, ai, Hx, jz, cu, h, fe, aq, NL, w, hR, B, bL, Dw, NQ, fM, yk, lH, jx, aA, cR, cz, v, C, V, q, eA, eJ, bgk, yw, aT, kf, cy, an, ae, af, hM, wr, il, Y, r, lo, en, t, iK, pb, ck, c, im, bt, ao, kh, jh, dG, eq, iJ, ad, dP, av, CJ, F, ds, ji, lQ, NM, NP, d, ff, eH, cr, aF, bz, bE, y, iu); #c1(RNF19A) c2(XP_011515258) c3(E3 ubiquitin-protein ligase RNF19A isoform X2) c4(308083, 308084, 308085, 308086) c5(308087, 308088, 308089, 308090, 308091, 308092, 308093, 308094, 308095, 308096, 308097, 308098, 308099, 308100, 308101, 308102, 308103, 308104, 308105, 308106, 308107, 308108, 308109, 308110) c6( ) c7(T, aC, ea, if, fd, bB, gf, lR, Lw, bv, xE, aE, rw, dV, K, aW, zC, g, as, eF, cm, bw, eM, cj, NO, Hb, m, NN, NK, dD, NJ, aj, BF, ct, qN, fr, cd, ai, Hx, jz, cU, h, fe, aq, NL, w, hR, B, bL, Dw, NQ, fM, yk, lH, jx, aA, CR, cz, v, C, V, q, eA, BJ, bgk, yw, aT, kf, cy, an, ae, af, hM, wr, il, Y, r, lo, en, t, ik, pb, ck, c, im, bt, ao, kh, jh, dG, eq, iJ, ad, dP, av, CJ, F, ds, ji, lQ, NM, NP, d, ff, eH, cr, aF, bz, bE, y, iu); #c1(RNF2) c2(XP_005245470) c3(E3 ubiquitin-protein ligase RING2 isoform X2) c4(266590) c5(266591, 266592, 266593, 266594, 266595, 266596) c6( ) c7(T, dA, Hn, n, fr, anu, ip, jz, ib, aE, py, Nn, aT, h, K, anw, anv, nO, aW, Y, kp, t, bw, bz, iP, kn, es, Ew, Dy, iA, lp, hk, m, anx, VB, hS); #c1(RNF2) c2(XP_011508154) c3(E3 ubiquitin-protein ligase RING2 isoform X1) c4(298639, 298640) c5(298641, 298642, 298643, 298644, 298645, 298646, 298647, 298648, 298649, 298650, 298651, 298652, 298653) c6( ) c7(T, dA, Hn, n, fr, anu, ip, jz, ib, aE, py, Nn, aT, h, K, anw, anv, nO, aW, Y, kp, t, bw, bz, iP, kn, es, Ew, Dy, iA, lp, hk, m, anx, VB, hS); #c1(RNF20) c2(NP_062538) c3(E3 ubiquitin-protein ligase BRE1A) c4(201839, 201840, 201841, 201842) c5(201843, 201844, 201845, 201846, 201847, 201848, 201849, 201850, 201851, 201852, 201853, 201854, 201855, 201856, 201857, 201858, 201859, 201860, 201861, 201862, 201863, 201864, 201865, 201866) c6( ) c7(Bl, ae, ad); #c1(RNF207) c2(XP_011539741) c3(RING finger protein 207 isoform X2) c4(337964) c5( ) c6( ) c7(EQ, mu); #c1(RNF207) c2(XP_011539742) c3(RING finger protein 207 isoform X3) c4( ) c5(337965, 337966, 337967, 337968, 337969) c6( ) c7(EQ, mu); #c1(RNF213) c2(NP_001243000) c3(E3 ubiquitin-protein ligase RNF213 isoform 3) c4(89514, 89515) c5(89516, 89517, 89518, 89519, 89520, 89521, 89522, 89523, 89524, 89525, 89526, 89527) c6( ) c7(aJ, bd, Hg, czR); #c1(RNF213) c2(NP_066005) c3(E3 ubiquitin-protein ligase RNF213 isoform 2) c4(206717) c5(206718, 206719, 206720, 206721, 206722, 206723) c6( ) c7(aJ, bd, Hg, czR); #c1(RNF213) c2(XP_005257603) c3(E3 ubiquitin-protein ligase RNF213 isoform X1) c4(275285, 275286) c5(275287, 275288, 275289, 275290, 275291, 275292, 275293, 275294, 275295, 275296, 275297, 275298) c6( ) c7(aJ, bd, Hg, czR); #c1(RNF214) c2(NP_997226) c3(RING finger protein 214 isoform 1) c4(265213, 265214) c5(265215, 265216, 265217, 265218, 265219, 265220, 265221, 265222, 265223, 265224, 265225, 265226, 265227, 265228, 265229, 265230) c6( ) c7(e); #c1(RNF216) c2(NP_996999) c3(E3 ubiquitin-protein ligase RNF216 isoform b) c4(264916) c5(264917, 264918, 264919, 264920, 264921, 264922) c6( ) c7(bEn, v, tk); #c1(RNF216) c2(XP_011513736) c3(E3 ubiquitin-protein ligase RNF216 isoform X1) c4(306035, 306036) c5(306037, 306038, 306039, 306040, 306041, 306042, 306043, 306044, 306045, 306046, 306047, 306048, 306049, 306050, 306051, 306052, 306053) c6( ) c7(bEn, v, tk); #c1(RNF216) c2(XP_011513737) c3(E3 ubiquitin-protein ligase RNF216 isoform X2) c4(306054) c5(306055) c6( ) c7(bEn, v, tk); #c1(RNF24) c2(NP_001127809) c3(RING finger protein 24 isoform 1) c4( ) c5(49265, 49266, 49267, 49268, 49269, 49270, 49271, 49272) c6( ) c7(oe); #c1(RNF24) c2(NP_001127810) c3(RING finger protein 24 isoform 2) c4(49273, 49274) c5(49275, 49276, 49277, 49278, 49279, 49280, 49281, 49282, 49283, 49284, 49285, 49286) c6( ) c7(oe); #c1(RNF24) c2(XP_011527448) c3(RING finger protein 24 isoform X3) c4( ) c5(322213) c6( ) c7(oe); #c1(RNF31) c2(NP_060469) c3(E3 ubiquitin-protein ligase RNF31) c4(196065, 196066, 196067, 196068) c5(196069, 196070, 196071, 196072, 196073, 196074, 196075, 196076, 196077, 196078, 196079, 196080, 196081, 196082, 196083, 196084, 196085, 196086) c6( ) c7(T, aT); #c1(RNF34) c2(NP_079402) c3(E3 ubiquitin-protein ligase RNF34 isoform 2) c4(217931) c5(217932, 217933, 217934, 217935, 217936, 217937) c6( ) c7(con, dA, bcb, ig, ht, CT, dV, CH, h, jh, bDl, ciP, eh, X, LH); #c1(RNF34) c2(NP_919247) c3(E3 ubiquitin-protein ligase RNF34 isoform 1) c4(258282) c5(258283, 258284, 258285, 258286, 258287, 258288, 258289, 258290, 258291, 258292) c6( ) c7(ccn, dA, bcb, ig, ht, CT, dV, CH, h, jh, bDl, ciP, eh, X, LH); #c1(RNF39) c2(NP_079512) c3(RING finger protein 39 isoform 1) c4(218453, 218454, 218455, 218456) c5(218457, 218458, 218459, 218460, 218461, 218462, 218463, 218464, 218465, 218466, 218467, 218468, 218469, 218470, 218471, 218472, 218473, 218474, 218475, 218476, 218477, 218478, 218479, 218480, 218481, 218482, 218483, 218484, 218485, 218486, 218487, 218488, 218489, 218490, 218491, 218492, 218493, 218494) c6( ) c7(h, hH, V, iQ, eZ); #c1(RNF39) c2(NP_739575) c3(RING finger protein 39 isoform 2) c4(247357, 247358, 247359, 247360, 247361, 247362) c5(247363, 247364, 247365, 247366, 247367, 247368, 247369, 247370, 247371, 247372, 247373, 247374, 247375, 247376, 247377, 247378, 247379, 247380, 247381, 247382, 247383, 247384, 247385, 247386, 247387, 247388, 247389, 247390, 247391, 247392, 247393, 247394, 247395, 247396, 247397, 247398, 247399, 247400, 247401, 247402, 247403, 247404, 247405, 247406, 247407, 247408, 247409, 247410, 247411, 247412, 247413, 247414, 247415, 247416, 247417, 247418, 247419, 247420, 247421, 247422, 247423, 247424, 247425, 247426, 247427, 247428, 247429, 247430) c6( ) c7(h, hH, V, iQ, eZ); #c1(RNF4) c2(NP_001171939) c3(E3 ubiquitin-protein ligase RNF4 isoform 2) c4(72039, 72040, 72041) c5(72042, 72043, 72044, 72045, 72046, 72047, 72048, 72049, 72050, 72051, 72052, 72053, 72054, 72055, 72056, 72057, 72058, 72059) c6( ) c7(rc, oe, jj, aed, kS, pi); #c1(RNF4) c2(NP_002929) c3(E3 ubiquitin-protein ligase RNF4 isoform 1) c4(133670, 133671) c5(133672, 133673, 133674, 133675, 133676, 133677, 133678, 133679, 133680, 133681, 133682, 133683, 133684, 133685, 133686, 133687, 133688, 133689, 133690, 133691, 133692) c6( ) c7(rc, oe, jj, aed, kS, pi); #c1(RNF40) c2(NP_001193963) c3(E3 ubiquitin-protein ligase BRE1B isoform 3) c4(83350) c5(83351, 83352, 83353, 83354, 83355, 83356, 83357, 83358, 83359, 83360, 83361, 83362, 83363) c6( ) c7(ad, bP, K, ae, er, bN); #c1(RNF40) c2(NP_055586) c3(E3 ubiquitin-protein ligase BRE1B isoform 1) c4(182122, 182123, 182124) c5(182125, 182126, 182127, 182128, 182129, 182130, 182131, 182132, 182133, 182134, 182135, 182136) c6( ) c7(ad, bP, K, ae, er, bN); #c1(RNF40) c2(XP_011544301) c3(E3 ubiquitin-protein ligase BRE1B isoform X3) c4(343453) c5( ) c6( ) c7(ad, bP, K, ae, er, bN); #c1(RNF40) c2(XP_011544302) c3(E3 ubiquitin-protein ligase BRE1B isoform X4) c4( ) c5(343454, 343455, 343456, 343457, 343458, 343459) c6( ) c7(ad, bP, K, ae, er, bN); #c1(RNF41) c2(NP_001229755) c3(E3 ubiquitin-protein ligase NRDP1 isoform 1) c4(85113, 85114, 85115, 85116) c5(85117, 85118, 85119, 85120, 85121, 85122, 85123, 85124, 85125, 85126, 85127, 85128, 85129, 85130, 85131, 85132, 85133, 85134, 85135, 85136, 85137, 85138, 85139, 85140, 85141, 85142, 85143, 85144, 85145, 85146, 85147) c6( ) c7(aW, bO, cj, r, aT, bk, an, he, q, hq); #c1(RNF41) c2(XP_011536037) c3(E3 ubiquitin-protein ligase NRDP1 isoform X1) c4(333333, 333334) c5(333335, 333336, 333337, 333338, 333339, 333340, 333341, 333342, 333343, 333344) c6( ) c7(aW, bO, cj, r, aT, bK, an, he, q, hq); #c1(RNF43) c2(NP_060233) c3(E3 ubiquitin-protein ligase RNF43 precursor) c4(194890, 194891) c5(194892, 194893, 194894, 194895, 194896, 194897, 194898, 194899, 194900, 194901, 194902, 194903) c6( ) c7(T, aC, jz, bE, hL, bcd, r, aA, bB, ci, m, q, g, t, B, cI); #c1(RNF44) c2(XP_005265902) c3(RING finger protein 44 isoform X4) c4( ) c5(279837, 279838, 279839, 279840, 279841) c6( ) c7(gv); #c1(RNF44) c2(XP_011532768) c3(RING finger protein 44 isoform X1) c4(328850, 328851) c5(328852, 328853, 328854, 328855, 328856, 328857, 328858) c6( ) c7(gv); #c1(RNF5) c2(NP_008844) c3(E3 ubiquitin-protein ligase RNF5) c4(168995, 168996) c5(168997, 168998, 168999, 169000, 169001, 169002, 169003, 169004, 169005, 169006, 169007, 169008, 169009, 169010, 169011, 169012, 169013, 169014, 169015, 169016, 169017, 169018, 169019, 169020, 169021, 169022, 169023, 169024, 169025, 169026, 169027, 169028, 169029, 169030, 169031, 169032, 169033, 169034, 169035, 169036, 169037, 169038, 169039, 169040, 169041, 169042, 169043, 169044, 169045, 169046, 169047, 169048) c6( ) c7(es, A, W, aW, Y, cr, aT, eZ, lo, K, cp); #c1(RNF6) c2(NP_005968) c3(E3 ubiquitin-protein ligase RNF6) c4(160840, 160841) c5(160842, 160843, 160844, 160845, 160846, 160847, 160848, 160849, 160850, 160851, 160852, 160853, 160854, 160855, 160856, 160857, 160858, 160859, 160860, 160861) c6( ) c7(cpE, ht, it, bee, ae, ad); #c1(RNF7) c2(NP_001188299) c3(RING-box protein 2 isoform 4) c4(79023) c5(79024, 79025, 79026, 79027, 79028, 79029) c6( ) c7(A, bE, tp, LX, e, qr, bgg, cy, K, bFs, lf, Dm, LT); #c1(RNF7) c2(NP_055060) c3(RING-box protein 2 isoform 1) c4(178661) c5(178662, 178663, 178664, 178665, 178666, 178667) c6( ) c7(A, bE, tp, LX, e, qr, bgg, cy, K, bFs, lf, Dm, LT); #c1(RNF7) c2(NP_899060) c3(RING-box protein 2 isoform 3) c4(257931) c5(257932, 257933, 257934, 257935, 257936, 257937) c6( ) c7(A, bE, tp, LX, e, qr, bgg, cy, K, bFs, lf, Dm, LT); #c1(RNF8) c2(NP_003949) c3(E3 ubiquitin-protein ligase RNF8 isoform 1) c4(142398, 142399) c5(142400, 142401, 142402, 142403, 142404, 142405, 142406) c6( ) c7(os, ac, kP, oZ, cm, bN); #c1(RNF8) c2(NP_898901) c3(E3 ubiquitin-protein ligase RNF8 isoform 2) c4(257858) c5(257859, 257860, 257861, 257862, 257863, 257864) c6( ) c7(os, ac, kP, oZ, cm, bN); #c1(RNF8) c2(XP_006715305) c3(E3 ubiquitin-protein ligase RNF8 isoform X2) c4( ) c5(289491, 289492, 289493, 289494, 289495, 289496) c6( ) c7(os, ac, kP, oZ, cm, bN); #c1(RNGTT) c2(NP_001273355) c3(mRNA-capping enzyme isoform b) c4(104719) c5(104720, 104721, 104722, 104723, 104724, 104725, 104726) c6( ) c7(iR, aq, T, B); #c1(RNGTT) c2(NP_001273357) c3(mRNA-capping enzyme isoform c) c4(104727) c5(104728, 104729, 104730, 104731, 104732) c6( ) c7(iR, aq, T, B); #c1(RNGTT) c2(NP_003791) c3(mRNA-capping enzyme isoform a) c4(140859, 140860, 140861) c5(140862, 140863, 140864, 140865, 140866, 140867, 140868, 140869, 140870, 140871, 140872, 140873, 140874, 140875, 140876, 140877, 140878, 140879) c6( ) c7(iR, aq, T, B); #c1(RNH1) c2(XP_011518565) c3(ribonuclease inhibitor isoform X1) c4(312631, 312632, 312633, 312634, 312635) c5(312636, 312637, 312638, 312639, 312640, 312641, 312642, 312643, 312644, 312645, 312646, 312647, 312648, 312649, 312650, 312651, 312652, 312653, 312654, 312655, 312656, 312657, 312658, 312659, 312660, 312661, 312662, 312663, 312664, 312665, 312666, 312667, 312668, 312669, 312670, 312671, 312672, 312673, 312674, 312675, 312676, 312677, 312678, 312679, 312680, 312681, 312682, 312683, 312684, 312685, 312686, 312687, 312688, 312689, 312690, 312691, 312692, 312693, 312694, 312695, 312696, 312697, 312698, 312699, 312700, 312701, 312702, 312703, 312704, 312705, 312706, 312707, 312708, 312709, 312710, 312711, 312712, 312713, 312714, 312715, 312716) c6(312717, 312718, 312719, 312720, 312721, 312722, 312723, 312724, 312725, 312726, 312727, 312728, 312729, 312730, 312731, 312732, 312733, 312734, 312735, 312736, 312737, 312738, 312739) c7(ck, T, sS, hd, nB, aW, Y, eL, Qo, Lf, cj, aT, aA, h, dl, aF, bu, bz, zn); #c1(RNLS) c2(NP_001026879) c3(renalase isoform 1 precursor) c4(29785) c5(29786, 29787, 29788, 29789, 29790, 29791) c6( ) c7(fU, A, ac, Hm, M, cz, dy, uD, cd, qa); #c1(RNLS) c2(XP_005270006) c3(renalase isoform X6) c4( ) c5(283030) c6( ) c7(fU, A, ac, Hm, M, cz, dy, uD, cd, qa); #c1(RNLS) c2(XP_005270007) c3(renalase isoform X7) c4(283031) c5(283032, 283033, 283034, 283035, 283036, 283037) c6( ) c7(fU, A, ac, Hm, M, cz, dy, uD, cd, qa); #c1(RNLS) c2(XP_011538227) c3(renalase isoform X1) c4(335835) c5(335836, 335837, 335838, 335839, 335840, 335841) c6( ) c7(fU, A, ac, Hm, M, cz, dy, uD, cd, qa); #c1(RNMT) c2(NP_003790) c3(mRNA cap guanine-N7 methyltransferase) c4(140835, 140836) c5(140837, 140838, 140839, 140840, 140841, 140842, 140843, 140844, 140845, 140846, 140847, 140848, 140849, 140850, 140851, 140852, 140853, 140854, 140855, 140856, 140857, 140858) c6( ) c7(T, aC, aCh, pf, bll, bO, fd, bB, ci, aY, bv, aE, Xc, pX, Qk, K, KC, eG, iE, aW, g, as, eF, ir, bw, QA, Qt, iP, eM, iA, lp, aep, m, Xu, ik, n, fr, rP, ft, gd, kH, dL, kj, aq, kp, Dy, SE, dB, ayS, hL, auh, it, B, dA, eR, jx, aA, aNG, lu, q, eA, aT, rz, ae, jf, s, nO, oV, Y, r, en, aDY, cl, t, Vi, iK, bN, Qi, nM, iM, np, bt, ao, VB, hS, id, LX, bXc, jh, KO, Sv, Fc, aue, ad, dP, av, hd, ib, py, Db, hP, d, bug, je, PE, ff, eH, gl, aF, adj, jb, bz, bF, es, RV, bE, lf, iu); #c1(RNMT) c2(XP_011524055) c3(mRNA cap guanine-N7 methyltransferase isoform X5) c4( ) c5(318643, 318644, 318645, 318646, 318647) c6( ) c7(T, aC, aCh, pf, bll, bO, fd, bB, ci, aY, bv, aE, Xc, pX, Qk, K, KC, eG, iE, aW, g, as, eF, ir, bw, QA, Qt, iP, eM, iA, lp, aep, m, Xu, ik, n, fr, rP, ft, gd, kH, dL, kj, aq, kp, Qy, SE, dB, ayS, hL, auh, it, B, dA, eR, jx, aA, aNG, lu, q, eA, aT, rz, ae, jf, s, nO, oV, Y, r, en, aDY, cl, t, Vi, ik, bN, Qi, nM, iM, np, bt, ao, VB, hS, id, LX, bXc, jh, KO, Sv, Fc, aue, ad, dP, av, hd, ib, py, Db, hP, d, bug, je, PE, ff, eH, gl, aF, adj, jb, bz, bF, es, RV, bE, lf, iu); #c1(RNPEP) c2(NP_064601) c3(aminopeptidase B) c4(203236, 203237, 203238) c5(203239, 203240, 203241, 203242, 203243, 203244, 203245, 203246, 203247, 203248, 203249, 203250) c6( ) c7(aC, hd, WA, ahL, hQ, cz, cm, fr, jj, as, eF, zP, kS); #c1(RNPEP) c2(XP_005245475) c3(aminopeptidase B isoform X1) c4(266597) c5(266598, 266599, 266600, 266601, 266602, 266603) c6( ) c7(aC, hd, WA, ahL, hQ, cz, cm, fr, jj, as, eF, zP, kS); #c1(RNPEP) c2(XP_005245476) c3(aminopeptidase B isoform X2) c4( ) c5(266604, 266605, 266606, 266607, 266608, 266609) c6( ) c7(aC, hd, WA, ahL, hQ, cz, cm, fr, jj, as, eF, zP, kS); #c1 (RNPEPL1) c2(NP_060696) c3(arginyl aminopeptidase-like 1) c4(197279) c5(197280, 197281, 197282, 197283, 197284, 197285) c6( ) c7(bL, A, cz, j, bK, y); #c1(RNPS1) c2(NP_001273555) c3(RNA-binding protein with serine-rich domain 1 isoform b) c4( ) c5(105217, 105218, 105219, 105220, 105221, 105222, 105223, 105224, 105225, 105226, 105227, 105228, 105229, 105230, 105231, 105232) c6( ) c7(T, bb, cA, SE, eM, ahk, aT, h, lR, nA, pt, pb); #c1(RNPS1) c2(NP_001273556) c3(RNA-binding protein with serine-rich domain 1 isoform c) c4( ) c5(105233) c6( ) c7(T, bb, cA, SE, eM, ahk, aT, h, lR, nA, pt, pb); #c1 (RNPS1) c2(XP_005255106) c3(RNA-binding protein with serine-rich domain 1 isoform X1) c4(273549) c5( ) c6( ) c7(T, bb, cA, SE, eM, ahk, aT, h, lR, nA, pt, pb); #c1(ROBO1) c2(NP_001139317) c3(roundabout homolog 1 isoform d) c4(59156) c5(59157, 59158, 59159, 59160, 59161, 59162) c6( ) c7(bM, T, Mb, dA, arS, aw, VT, aA, hy, v, fr, qN, alP, tK, q, ad, eA, av, ft, aT, io, ae, Ha, ahE, jf, lR, aW, Y, r, aq, kp, aF, ahL, g, iR, jb, bz, bN, es, W, eM, Hs, sv, yA, bdF, B); #c1(ROBO1) c2(NP_002932) c3(roundabout homolog 1 isoform a precursor) c4(133707, 133708, 133709) c5(133710, 133711, 133712, 133713, 133714, 133715, 133716, 133717, 133718, 133719, 133720, 133721) c6( ) c7(bM, T, Mb, dA, arS, aw, VT, aA, hy, v, fr, qN, alP, tK, q, ad, eA, av, ft, aT, io, ae, Ha, ahE, jf, lR, aW, Y, r, aq, kp, aF, ahl, g, iR, jb, bz, bN, es, W, eM, Hs, sv, yA, bdF, B); #c1(ROBO1) c2(NP_598334) c3(roundabout homolog 1 isoform b) c4(235419, 235420) c5(235421, 235422, 235423, 235424, 235425, 235426, 235427, 235428, 235429, 235430, 235431, 235432) c6( ) c7(bM, T, Mb, dA, arS, aw, VT, aA, hy, v, fr, qN, alP, tK, q, ad, eA, av, ft, aT, io, ae, Ha, ahE, jf, lR, aW, Y, r, aq, kp, aF, ahl, g, iR, jb, bz, bN, es, W, eM, Hs, sv, yA, bdF, B); #c1(ROBO1) c2(XP_006713340) c3(roundabout homolog 1 isoform X4) c4( ) c5(287141, 287142, 287143, 287144, 287145, 287146) c6( ) c7(bM, T, Mb, dA, arS, aw, VT, aA, hy, v, fr, qN, alP, tK, q, ad, eA, av, ft, aT, io, ae, Ha, ahE, jf, lR, aW, Y, r, aq, kp, aF, ahl, g, iR, jb, bz, bN, es, W, eM, Hs, sv, yA, bdF, B); #c1(ROBO2) c2(NP_001122401) c3(roundabout homolog 2 isoform ROBO2a) c4(46757, 46758) c5(46759, 46760, 46761, 46762, 46763, 46764, 46765, 46766, 46767, 46768, 46769, 46770, 46771, 46772, 46773, 46774) c6( ) c7(Kc, T, tY, dY, jz, wG, tL, oe, aw, czS, bcd, r, aur, aj, qN, cm, bN); #c1(ROBO2) c2(NP_001276968) c3(roundabout homolog 2 isoform 3 precursor) c4(108855) c5(108856, 108857, 108858, 108859, 108860, 108861) c6( ) c7(Kc, T, tY, dY, jz, wG, tL, oe, aw, czS, bcd, r, aur, aj, qN, cm, bN); #c1(ROBO2) c2(NP_002933) c3(roundabout homolog 2 isoform ROBO2b precursor) c4(133722) c5(133723, 133724, 133725, 133726, 133727, 133728) c6( ) c7(Kc, T, tY, dY, jz, wG, tL, oe, aw, czS, bcd, r, aur, aj, qN, cm, bN); #c1(ROBO2) c2(XP_011532283) c3(roundabout homolog 2 isoform X1) c4(328350) c5( ) c6( ) c7(Kc, T, tY, dY, jz, wG, tL, oe, aw, czS, bcd, r, aur, aj, qN, cm, bN); #c1(ROBO3) c2(NP_071765) c3(roundabout homolog 3 precursor) c4(212005, 212006) c5(212007, 212008, 212009, 212010, 212011, 212012, 212013, 212014, 212015, 212016, 212017, 212018, 212019, 212020, 212021) c6( ) c7(Dw, T, dD, czU, ea, if, jx, aA, j, eq, qN, fr, eW, MR, Dq, ds, rC, cU, h, anq, K, afu, A, d, aCg, bes, Y, yL, apn, eZ, ff, aiT, lo, kp, aF, La, en, CQ, bQ, t, ik, awi, dr, czT, bN, Bv, ac, lq, NL, AH, Uq, aiM, bel, y); #c1(ROBO3) c2(XP_011541255) c3(roundabout homolog 3 isoform X1) c4(339911) c5(339912, 339913, 339914, 339915, 339916) c6( ) c7(Dw, T, dD, czU, ea, if, jx, aA, j, eq, qN, fr, eW, MR, Dq, ds, rC, cU, h, anq, K, afu, A, d, aCg, bes, Y, yL, apn, eZ, ff, aiT, lo, kp, aF, La, en, CQ, bQ, t, ik, awi, dr, czT, bN, Bv, ac, lq, NL, AH, Uq, aiM, bel, y); #c1(ROBO4) c2(NP_001288017) c3(roundabout homolog 4 isoform 2) c4(113430, 113431) c5( ) c6( ) c7(c, eM, fG, yL, cz, kp, B, ad, bN); #c1(ROBO4) c2(NP_061928) c3(roundabout homolog 4 isoform 1 precursor) c4(201175) c5(201176, 201177, 201178, 201179, 201180, 201181, 201182, 201183, 201184, 201185, 201186, 201187, 201188, 201189, 201190, 201191, 201192, 201193) c6( ) c7(c, eM, fG, yL, cz, kp, B, ad, bN); #c1(ROBO4) c2(XP_006718924) c3(roundabout homolog 4 isoform X1) c4( ) c5(292113) c6( ) c7(c, eM, fG, yL, cz, kp, B, ad, bN); #c1(ROCK1) c2(NP_005397) c3(rho-associated protein kinase 1) c4(155693) c5(155694, 155695, 155696, 155697, 155698, 155699) c6( ) c7(bL, uB, T, lD, aC, ea, e, aA, cz, mw, aj, dG, j, v, bB, Lw, eb, fr, q, J, ad, El, dP, eA, bv, av, iu, aE, py, cc, sN, acA, ds, oj, aT, kf, h, dz, dL, K, hH, ae, af, eL, ly, eG, d, bf, aW, eH, r, Qm, aF, as, dw, t, eF, cm, lx, dR, xd, eM, bE, X, nw, m, fD, ei, lC, bt, B); #c1(ROCK2) c2(NP_004841) c3(rho-associated protein kinase 2) c4(150188, 150189, 150190) c5(150191, 150192, 150193, 150194, 150195, 150196, 150197, 150198, 150199, 150200, 150201, 150202, 150203, 150204, 150205, 150206, 150207, 150208) c6( ) c7(lD, T, e, al, cz, eb, j, eA, aT, h, By, K, hH, wC, ly, d, aW, uC, qa, lx, bF, xd, bE, cj, lC, B); #c1(ROCK2) c2(XP_011508719) c3(rho-associated protein kinase 2 isoform X2) c4( ) c5(299340) c6( ) c7(lD, T, e, al, cz, eb, j, eA, aT, h, By, K, hH, wC, ly, d, aW, uC, qa, lx, bF, xd, bE, cj, lC, B); #c1(ROGD1) c2(NP_078865) c3(protein rogdi homolog) c4(215832) c5(215833, 215834, 215835, 215836, 215837, 215838) c6( ) c7(czV); #c1(ROM1) c2(NP_000318) c3(rod outer segment membrane protein 1) c4(7006) c5(7007, 7008, 7009, 7010, 7011, 7012) c6( ) c7(T, xX, lR, BO, W, z, aW, Sr, nx, aDj, aT, dr, bxi, nw, nk, rz, nC, cap, czW, de); #c1(ROPN1) c2(XP_011511238) c3(ropporin-IA isoform X2) c4( ) c5(301719, 301720, 301721, 301722, 301723, 301724, 301725, 301726, 301727, 301728, 301729, 301730, 301731, 301732, 301733, 301734, 301735, 301736, 301737, 301738, 301739, 301740, 301741, 301742) c6( ) c7(eu, Ea, ac, M, cz); #c1(ROPN1B) c2(XP_005247195) c3(ropporin-18 isoform X3) c4( ) c5(267690, 267691, 267692, 267693, 267694, 267695, 267696, 267697) c6( ) c7(asG, Y); #c1(ROPN1B) c2(XP_011510753) c3(ropporin-1B isoform X2) c4( ) c5(301174, 301175, 301176, 301177, 301178, 301179, 301180, 301181, 301182, 301183, 301184, 301185) c6( ) c7(asG, Y); #c1(ROPN1L) c2(NP_114122) c3(ropporin-1-like protein) c4(222242) c5(222243, 222244, 222245, 222246, 222247, 222248, 222249, 222250, 222251, 222252) c6( ) c7(x, y, l, A, c, u, w, h, n, v, j, m, k, t, fq, o, p); #c1(ROR1) c2(XP_011539828) c3(tyrosine-protein kinase transmembrane receptor ROR1 isoform X1) c4( ) c5(338062, 338063, 338064, 338065, 338066) c6( ) c7(ck, es, T, iu, x, eM, iA, Y, aw, cj, aT, Hn, aj, aF, bK, k, ei, im, cd, er, Qt); #c1(ROR2) c2(NP_004551) c3(tyrosine-protein kinase transmembrane receptor ROR2 precursor) c4(147580) c5(147581, 147582, 147583, 147584, 147585, 147586) c6( ) c7(T, rV, acl, fd, Bm, fr, ci, zU, ad, zw, hW, fb, ae, bA, cp, jf, cml, Y, eH, rs, eF, Qt, dr, ac, rq, czX, dy, YH); #c1(RORA) c2(NP_002934) c3(nuclear receptor ROR-alpha isoform c) c4(133729) c5(133730, 133731, 133732, 133733, 133734, 133735, 133736) c6( ) c7(bL, bM, T, dD, rV, acl, fd, aA, Bm, Hn, im, bS, eq, jE, ct, ci, zU, bX, fr, ad, er, hX, x, iu, Yn, zw, hW, aT, ta, cy, bK, K, lQ, ji, fb, nJ, ae, Um, cp, bA, jf, cml, aW, Y, eH, hl, dq, aq, rs, af, Z, aGj, eF, Qt, dr, bN, aPY, ck, es, W, rq, eM, iA, Ei, cj, czX, k, cP, ei, GF, YH, B); #c1(RORA) c2(NP_599022) c3(nuclear receptor ROR-alpha isoform b) c4(235569) c5(235570, 235571, 235572, 235573, 235574, 235575, 235576) c6( ) c7(bL, bM, T, dD, rV, acl, fd, aA, Bm, Hn, im, bS, eq, jE, ct, ci, zU, bX, fr, ad, er, hX, x, iu, Yn, zw, hW, aT, ta, cy, bK, K, lQ, ji, fb, nJ, ae, Um, cp, bA, jf, cml, aW, Y, eH, hl, dq, aq, rs, af, Z, aGj, eF, Qt, dr, bN, aPY, ck, es, W, rq, eM, iA, Ei, cj, czX, k, cP, ei, GF, YH, B); #c1(RORA) c2(NP_599023) c3(nuclear receptor ROR-alpha isoform a) c4(235577) c5(235578, 235579, 235580, 235581, 235582, 235583, 235584) c6( ) c7(bL, bM, T, dD, rV, acl, fd, aA, Bm, Hn, im, bS, eq, jE, ct, ci, zU, bX, fr, ad, er, hX, x, iu, Yn, zw, hW, aT, ta, cy, bK, K, lQ, ji, fb, nJ, ae, Um, cp, bA, jf, cml, aW, Y, eH, hl, dq, aq, rs, af, Z, aGj, eF, Qt, dr, bN, aPY, ck, es, W, rq, eM, iA, Ei, cj, czX, k, cP, ei, GF, YH, B); #c1(RORA) c2(NP_599024) c3(nuclear receptor ROR-alpha isoform d) c4(235585) c5(235586, 235587, 235588, 235589, 235590, 235591, 235592) c6( ) c7(bL, bM, T, dD, rV, acl, fd, aA, Bm, Hn, im, bS, eq, jE, ct, ci, zU, bX, fr, ad, er, hX, x, iu, Yn, zw, hW, aT, ta, cy, bK, K, lQ, ji, fb, nJ, ae, Um, cp, bA, jf, cml, aW, Y, eH, hl, dq, aq, rs, af, Z, aGj, eF, Qt, dr, bN, aPY, ck, es, W, rq, eM, iA, Ei, cj, czX, k, cP, ei, GF, YH, B); #c1(RORA) c2(XP_011520177) c3(nuclear receptor ROR-alpha isoform X3) c4( ) c5(315048) c6( ) c7(bL, bM, T, dD, rV, acl, fd, aA, Bm, Hn, im, bS, eq, jE, ct, ci, zU, bX, fr, ad, er, hX, x, iu, Yn, zw, hW, aT, ta, cy, bK, K, lQ, ji, fb, nJ, ae, Um, cp, bA, jf, cml, aW, Y, eH, hl, dq, aq, rs, aF, Z, aGj, eF, Qt, dr, bN, aPY, ck, es, W, rq, eM, iA, Ei, cj, czX, k, cP, ei, GF, YH, B); #c1(RORB) c2(NP_008845) c3(nuclear receptor ROR-beta) c4(169049, 169050) c5(169051, 169052, 169053, 169054, 169055, 169056, 169057, 169058, 169059, 169060, 169061, 169062, 169063) c6( ) c7(aPY, bk, dP); #c1(RORC) c2(NP_001001523) c3(nuclear receptor ROR-gamma isoform b) c4(18602) c5(18603, 18604, 18605, 18606, 18607, 18608, 18609) c6( ) c7(bL, T, OG, cc, bei, pf, aW, JW, wz, aT, Vv, n, r, v, dW, yO, ae, q, dr, ad); #c1(RORC) c2(NP_005051) c3(nuclear receptor ROR-gamma isoform a) c4(152460) c5(152461, 152462, 152463, 152464, 152465, 152466, 152467) c6( ) c7(bL, T, OG, cc, bei, pf, aW, JW, wz, aT, Vv, n, r, v, dW, yO, ae, q, dr, ad); #c1(RORC) c2(XP_006711547) c3(nuclear receptor ROR-gamma isoform X1) c4(286112) c5(286113, 286114, 286115, 286116, 286117, 286118) c6( ) c7(bL, T, OG, cc, bei, pf, aW, JW, wz, aT, Vv, n, r, v, dW, yO, ae, q, dr, ad); #c1(ROS1) c2(NP_002935) c3(proto-oncogene tyrosine-protein kinase ROS precursor) c4(133737) c5(133738, 133739, 133740, 133741, 133742, 133743) c6( ) c7(Mb, T, eR, e, aw, aA, cz, n, j, ci, q, eA, ip, av, jz, aT, dL, eu, azH, d, Jl, aW, Y, r, aF, g, t, bz, ek, iP, kT, ac, eM, Vy, at, m, ei, iu); #c1(ROS1) c2(XP_006715611) c3(proto-oncogene tyrosine-protein kinase ROS isoform X5) c4(289640) c5(289641, 289642, 289643, 289644, 289645, 289646) c6( ) c7(Mb, T, eR, e, aw, aA, cz, n, j, ci, q, eA, ip, av, jz, aT, dl, eu, azH, d, Jl, aW, Y, r, aF, g, t, bz, ek, iP, kT, ac, eM, Vy, at, m, ei, iu); #c1 (ROS1) c2(XP_011534357) c3(proto-oncogene tyrosine-protein kinase ROS isoform X8) c4(330731) c5(330732, 330733, 330734, 330735, 330736, 330737) c6( ) c7(Mb, T, eR, e, aw, aA, cz, n, j, ci, q, eA, ip, av, jz, aT, dL, eu, azH, d, Jl, aW, Y, r, aF, g, t, bz, ek, iP, kT, ac, eM, Vy, at, m, ei, iu); #c1(RP1) c2(NP_006260) c3(oxygen-regulated protein 1) c4(163350) c5(163351, 163352, 163353, 163354, 163355, 163356, 163357) c6( ) c7(xX, lR, cO, r, nw, nk, cm, nC, q, RE); #c1(RP1L1) c2(NP_849188) c3(retinitis pigmentosa 1-like 1 protein) c4(254453) c5(254454, 254455, 254456, 254457, 254458, 254459, 254460) c6( ) c7(csr, W, nC, Sr, lR); #c1(RP2) c2(NP_008846) c3 (protein XRP2) c4(169064) c5(169065, 169066, 169067, 169068, 169069, 169070) c6( ) c7(afR, kl, xX, czZ, lR, nx, czY, Be, nw, eQ, HU, ayj, nC); #c1(RP9) c2(NP_976033) c3(retinitis pigmentosa 9 protein) c4(262830) c5(262831, 262832, 262833, 262834, 262835, 262836) c6( ) c7(T, b, xX, lR, if, aoA, cAa, qN, i, nC, GR); #c1(RPA1) c2(NP_002936) c3(replication protein A 70 kDa DNA-binding subunit) c4(133744) c5(133745, 133746, 133747, 133748, 133749, 133750) c6( ) c7(eW, T, wr, BN, ac, afl, aT, Nl, hy, dL, j, Kh, xH, V, ne); #c1(RPA2) c2(NP_001284487) c3(replication protein A 32 kDa subunit isoform 3) c4(111895, 111896, 111897, 111898) c5(111899, 111900, 111901, 111902, 111903, 111904) c6( ) c7(Bl, T, aiB, qt, aW, aT, cX, dL, eS, K, V); #c1(RPA3) c2(NP_002938) c3(replication protein A 14 kDa subunit) c4(133751) c5(133752, 133753, 133754, 133755, 133756, 133757, 133758, 133759, 133760, 133761) c6( ) c7(V, dL, aw, aT); #c1(RPA4) c2(NP_037479) c3(replication protein A 30 kDa subunit) c4(175738) c5(175739, 175740, 175741, 175742, 175743, 175744) c6( ) c7(dL, c); #c1(RPAIN) c2(NP_001028174) c3(RPA-interacting protein isoform b) c4(30344, 30345, 30346) c5(30347, 30348, 30349, 30350, 30351, 30352) c6( ) c7(dA, eM, aW, bo, yG, aT, aj, fD, EA, y, J); #c1(RPAIN) c2(NP_001153715) c3(RPA-interacting protein isoform a) c4(60890) c5(60891, 60892, 60893, 60894, 60895, 60896) c6( ) c7(dA, eM, aW, bo, yG, aT, aj, fD, EA, y, J); #c1(RPAIN) c2(NP_001153716) c3(RPA-interacting protein isoform c) c4(60897) c5(60898, 60899, 60900, 60901, 60902, 60903) c6( ) c7(dA, eM, aW, bo, yG, aT, aj, fD, EA, y, J); #c1(RPAIN) c2(NP_001153718) c3(RPA-interacting protein isoform d) c4(60904) c5(60905, 60906, 60907, 60908, 60909, 60910, 60911, 60912, 60913, 60914, 60915, 60916) c6( ) c7(dA, eM, aW, bo, yG, aT, aj, fD, EA, y, J); #c1(RPAIN) c2(NP_001153738) c3(RPA-interacting protein isoform e) c4(60965) c5(60966, 60967, 60968, 60969, 60970, 60971, 60972, 60973, 60974, 60975, 60976, 60977) c6( ) c7(dA, eM, aW, bo, yG, aT, aj, fD, EA, y, J); #c1(RPAP1) c2(NP_056355) c3(RNA polymerase II-associated protein 1) c4(187233) c5(187234, 187235, 187236, 187237, 187238, 187239) c6( ) c7(aW, aT); #c1(RPE) c2(NP_001265214) c3(ribulose-phosphate 3-epimerase isoform 3) c4(97950) c5(97951, 97952, 97953, 97954, 97955, 97956) c6( ) c7(bGg, agS, T, AT, bF, lR, nb, W, Ha, aQx, aqV, aVu, Hs, nw, ey, K, db, aqY, Uk); #c1(RPE) c2(NP_008847) c3(ribulose-phosphate 3-epimerase isoform 2) c4(169071, 169072) c5(169073, 169074, 169075, 169076, 169077, 169078, 169079, 169080, 169081, 169082, 169083, 169084, 169085, 169086, 169087, 169088, 169089, 169090) c6( ) c7(bGg, agS, T, AT, bF, lR, nb, W, Ha, aQx, aqV, aVu, Hs, nw, ey, K, db, aqY, Uk); #c1 (RPE) c2(NP_954699) c3(ribulose-phosphate 3-epimerase isoform 1) c4(261282, 261283) c5(261284, 261285, 261286, 261287, 261288, 261289, 261290, 261291, 261292, 261293, 261294, 261295, 261296, 261297) c6( ) c7(bGg, agS, T, AT, bF, lR, nb, W, Ha, aQx, aqV, aVu, Hs, nw, ey, K, db, aqY, Uk); #c1(RPGR) c2(NP_000319) c3(X-linked retinitis pigmentosa GTPase regulator isoform A) c4(7013, 7014, 7015) c5(7016, 7017, 7018, 7019, 7020, 7021, 7022, 7023, 7024, 7025, 7026, 7027) c6( ) c7(nx, cAd, jA, Sp, HU, Nh, afR, afm, bgm, K, MG, li, xX, lR, Pf, cAc, cAb, czY, aVu, nC, Ud, ayj, W, cnj, nb, U, Be, nw, OL, oQ, bgv, de); #c1(RPGR) c2(NP_001030025) c3(X-linked retinitis pigmentosa GTPase regulator isoform C) c4(30888, 30889, 30890) c5(30891, 30892, 30893, 30894, 30895, 30896, 30897, 30898, 30899, 30900, 30901, 30902, 30903, 30904, 30905, 30906, 30907, 30908, 30909, 30910) c6( ) c7(nx, cAd, jA, Sp, HU, Nh, afR, afm, bgm, K, MG, li, xX, lR, Pf, cAc, cAb, czY, aVu, nC, Ud, ayj, W, cnj, nb, U, Be, nw, OL, oQ, bgv, de); #c1(RPGR) c2(XP_005272690) c3(X-linked retinitis pigmentosa GTPase regulator isoform X2) c4( ) c5(284143, 284144, 284145, 284146, 284147, 284148) c6( ) c7(nx, cAd, jA, Sp, HU, Nh, afR, afm, bgm, K, MG, li, xX, lR, Pf, cAc, cAb, czY, aVu, nC, Ud, ayj, W, cnj, nb, U, Be, nw, OL, oQ, bgv, de); #c1 (RPGRIP1) c2(NP_065099) c3(X-linked retinitis pigmentosa GTPase regulator-interacting protein 1) c4(203926, 203927) c5(203928, 203929, 203930, 203931, 203932, 203933, 203934, 203935, 203936, 203937, 203938, 203939) c6( ) c7(cAe, KE, ago, cAf, lR, dH, nx, U, vm, nw, eQ, dw, nC, Nh, de); #c1(RPGRIP1) c2(XP_011535284) c3(X-linked retinitis pigmentosa GTPase regulator-interacting protein 1 isoform X8) c4(332296) c5(332297, 332298, 332299, 332300, 332301, 332302) c6( ) c7(cAe, KE, ago, cAf, lR, dH, nx, U, vm, nw, eQ, dw, nC, Nh, de); #c1(RPGRIP1) c2(XP_011535285) c3(X-linked retinitis pigmentosa GTPase regulator-interacting protein 1 isoform X9) c4(332303) c5(332304, 332305, 332306, 332307, 332308, 332309, 332310) c6( ) c7(cAe, KE, ago, cAf, lR, dH, nx, U, vm, nw, eQ, dw, nC, Nh, de); #c1(RPGRIP1L) c2(NP_001121369) c3(protein fantom isoform b) c4(45784) c5(45785, 45786, 45787, 45788, 45789, 45790) c6( ) c7(aBW, Ng, bL, alF, ago, cAh, Pf, cd, vm, nw, dy, cAg, bK, oQ, v, Sp, B); #c1(RPGRIP1L) c2(NP_056087) c3(protein fantom isoform a) c4(185635) c5(185636, 185637, 185638, 185639, 185640, 185641) c6( ) c7(aBW, Ng, bL, alF, ago, cAh, Pf, cd, vm, nw, dy, cAg, bK, oQ, v, Sp, B); #c1(RPH3A) c2(NP_001137326) c3(rabphilin-3A isoform 1) c4(55476, 55477, 55478, 55479, 55480, 55481, 55482, 55483, 55484, 55485, 55486, 55487) c5(55488, 55489, 55490, 55491, 55492, 55493, 55494, 55495, 55496, 55497, 55498, 55499, 55500, 55501, 55502, 55503, 55504, 55505, 55506, 55507, 55508, 55509, 55510, 55511, 55512, 55513, 55514, 55515, 55516, 55517, 55518, 55519, 55520, 55521, 55522, 55523, 55524, 55525, 55526, 55527, 55528, 55529, 55530, 55531, 55532, 55533, 55534, 55535, 55536, 55537, 55538, 55539, 55540, 55541, 55542, 55543, 55544, 55545, 55546, 55547, 55548, 55549, 55550, 55551, 55552, 55553, 55554, 55555, 55556, 55557, 55558, 55559, 55560, 55561, 55562, 55563, 55564, 55565, 55566, 55567, 55568, 55569, 55570, 55571, 55572, 55573, 55574, 55575, 55576, 55577, 55578, 55579, 55580) c6( ) c7(ak, rR, r, g, q, sj); #c1(RPH3A) c2(NP_055769) c3(rabphilin-3A isoform 2) c4(183385, 183386) c5(183387, 183388, 183389, 183390, 183391, 183392, 183393, 183394, 183395, 183396, 183397, 183398) c6( ) c7(ak, rR, r, g, q, sj); #c1(RPH3AL) c2(XP_011545302) c3(rab effector Noc2 isoform X5) c4(344075) c5(344076, 344077, 344078, 344079, 344080, 344081) c6( ) c7(qJ, bM, jf, r, fQ, m, an, t, q); #c1(RPH3AL) c2(XP_011545304) c3(rab effector Noc2 isoform X7) c4(344082) c5(344083, 344084, 344085, 344086, 344087, 344088, 344089, 344090, 344091, 344092, 344093, 344094, 344095, 344096) c6( ) c7(qJ, bM, jf, r, fQ, m, an, t, q); #c1(RPIA) c2(NP_653164) c3(ribose-5-phosphate isomerase) c4(238879, 238880) c5(238881, 238882, 238883, 238884, 238885, 238886, 238887, 238888, 238889, 238890, 238891, 238892) c6( ) c7(bxy, nM, cAi, nB, nv, fd, dq, K, bX, zz); #c1(RPL10) c2(NP_001243506) c3(60S ribosomal protein L10 isoform b) c4(90973, 90974) c5(90975, 90976, 90977, 90978, 90979, 90980) c6( ) c7(bN, T, aE, aW, bOL, eL, aT, KO, dL, m, g, er, ae, eA, cAj, ad); #c1(RPL10) c2(NP_001290554) c3(60S ribosomal protein L10 isoform a) c4(115520, 115521) c5(115522, 115523, 115524, 115525, 115526, 115527, 115528, 115529, 115530, 115531, 115532, 115533, 115534, 115535, 115536, 115537, 115538, 115539, 115540, 115541) c6( ) c7(bN, T, aE, aW, bOL, eL, aT, KO, dL, m, g, er, ae, eA, cAj, ad); #c1(RPL10) c2(NP_001290555) c3(60S ribosomal protein LIO isoform d) c4( ) c5(115542, 115543, 115544, 115545, 115546, 115547) c6( ) c7(bN, T, aE, aW, bOL, eL, aT, KO, dL, m, g, er, ae, eA, cAj, ad); #c1 (RPL10A) c2(NP_009035) c3(60S ribosomal protein L10a) c4(170342) c5(170343, 170344, 170345, 170346, 170347, 170348) c6( ) c7(ds); #c1(RPL10L) c2(NP_542784) c3(60S ribosomal protein LIO-like) c4(233075) c5(233076, 233077, 233078, 233079, 233080, 233081) c6( ) c7(t, j, m); #c1(RPL12) c2(NP_000967) c3(60S ribosomal protein L(2) c4(17757, 17758) c5(17759, 17760, 17761, 17762, 17763, 17764, 17765, 17766, 17767, 17768, 17769, 17770) c6( ) c7(Na, Nc, asD, ad); #c1(RPL13) c2(NP_001230060) c3(60S ribosomal protein L13 isoform 3) c4(85861) c5(85862, 85863, 85864, 85865, 85866, 85867) c6( ) c7(aW, ae, aA, ad, aT); #c1(RPL13) c2(NP_150254) c3(60S ribosomal protein L13 isoform 1) c4(228004) c5(228005, 228006, 228007, 228008, 228009, 228010, 228011, 228012, 228013, 228014) c6( ) c7(aW, ae, aA, ad, aT); #c1(RPL14) c2(NP_001030168) c3(60S ribosomal protein L14) c4(30947, 30948) c5(30949, 30950, 30951, 30952, 30953, 30954, 30955, 30956, 30957, 30958, 30959, 30960, 30961, 30962, 30963, 30964) c6( ) c7(bL, av, eM, aj, fr, an, ik, bz, eA, J); #c1(RPL15) c2(NP_001240313) c3(60S ribosomal protein L15 isoform 2) c4(88589, 88590) c5(88591, 88592, 88593, 88594, 88595, 88596, 88597, 88598, 88599, 88600, 88601) c6( ) c7(cAk, CJ, bN, aA); #c1(RPL15) c2(XP_011532288) c3(60S ribosomal protein L15 isoform X1) c4(328351, 328352, 328353, 328354, 328355) c5(328356, 328357, 328358, 328359, 328360, 328361, 328362, 328363, 328364, 328365, 328366, 328367, 328368, 328369, 328370, 328371, 328372, 328373, 328374, 328375, 328376, 328377, 328378, 328379, 328380, 328381, 328382, 328383, 328384, 328385, 328386, 328387, 328388, 328389, 328390, 328391, 328392, 328393, 328394, 328395, 328396, 328397, 328398, 328399, 328400, 328401, 328402) c6( ) c7(cAk, CJ, bN, aA); #c1(RPL17) c2(NP_001030 (78) c3(60S ribosomal protein L17 isoform a) c4(30973, 30974, 30975) c5(30976, 30977, 30978, 30979, 30980, 30981, 30982, 30983, 30984, 30985, 30986, 30987, 30988, 30989, 30990, 30991, 30992, 30993, 30994, 30995, 30996, 30997) c6( ) c7(T, A, iM, je, aW, Y, jx, eZ, aT, uo, kH, lo, aA, j, aF, h, y, hv); #c1(RPL17) c2(NP_001186274) c3(60S ribosomal protein L17 isoform b) c4( ) c5(77457, 77458, 77459, 77460, 77461, 77462, 77463, 77464) c6( ) c7(T, A, iM, je, aW, Y, jx, eZ, aT, uo, KH, lo, aA, j, aF, h, y, hv); #c1(RPL17-C18 orf32) c2(NP_001186284) c3(RPL17-C18 orf32 protein isoform 1) c4(77465) c5(77466, 77467, 77468, 77469, 77470, 77471) c6( ) c7(T, A, iM, je, aW, Y, jx, eZ, aT, uo, KH, lo, aA, dW, aF, h, y, hv); #c1(RPL18) c2(NP_000970) c3(60S ribosomal protein L18 isoform 1) c4(17796, 17797, 17798) c5(17799, 17800, 17801, 17802, 17803, 17804, 17805, 17806, 17807, 17808, 17809, 17810, 17811, 17812, 17813, 17814, 17815, 17816, 17817, 17818) c6( ) c7(aA); #c1(RPL18) c2(NP_001257419) c3(60S ribosomal protein L18 isoform 2) c4(95408) c5(95409, 95410, 95411, 95412, 95413, 95414) c6( ) c7(aA); #c1(RPL19) c2(NP_000972) c3(60S ribosomal protein L19) c4(17819) c5(17820, 17821, 17822, 17823, 17824, 17825) c6( ) c7(li, ae, aC, iu, eM, aW, r, aT, ci, bz, q, ad); #c1(RPL21) c2(NP_000973) c3(60S ribosomal protein L21) c4(17826) c5(17827, 17828, 17829, 17830, 17831, 17832, 17833, 17834, 17835, 17836, 17837, 17838, 17839, 17840, 17841, 17842, 17843, 17844) c6( ) c7(eW); #c1(RPL23) c2(NP_000969) c3(60S ribosomal protein L23) c4(17771, 17772, 17773) c5(17774, 17775, 17776, 17777, 17778, 17779, 17780, 17781, 17782, 17783, 17784, 17785, 17786, 17787, 17788, 17789, 17790, 17791, 17792, 17793, 17794, 17795) c6( ) c7(T, azm, Pg, aF, aA); #c1(RPL23A) c2(NP_000975) c3(60S ribosomal protein L23a) c4(17845, 17846, 17847, 17848, 17849) c5(17850, 17851, 17852, 17853, 17854, 17855, 17856, 17857, 17858, 17859, 17860, 17861, 17862, 17863, 17864, 17865, 17866, 17867, 17868, 17869, 17870, 17871, 17872, 17873, 17874, 17875, 17876, 17877, 17878, 17879) c6( ) c7(Y, an, qN, aT); #c1(RPL24) c2(NP_000977) c3(60S ribosomal protein L24) c4(17880, 17881) c5(17882, 17883, 17884, 17885, 17886, 17887, 17888, 17889, 17890, 17891, 17892, 17893) c6( ) c7(Sv); #c1(RPL27A) c2(NP_000981) c3(60S ribosomal protein L27a) c4(17912, 17913) c5(17914, 17915, 17916, 17917, 17918, 17919, 17920, 17921, 17922, 17923, 17924, 17925) c6( ) c7(oX, ky); #c1(RPL29) c2(NP_000983) c3(60S ribosomal protein L29) c4(17926) c5(17927, 17928, 17929, 17930, 17931, 17932, 17933, 17934, 17935, 17936, 17937, 17938, 17939, 17940, 17941, 17942, 17943, 17944, 17945, 17946, 17947, 17948, 17949, 17950, 17951, 17952) c6( ) c7(bL, eW, T, bE, aA, bB, aq, aj, v, aF, ci, K, q, X, J); #c1(RPL3) c2(NP_000958) c3(60S ribosomal protein L3 isoform a) c4(17694, 17695) c5(17696, 17697, 17698, 17699, 17700, 17701, 17702, 17703, 17704, 17705, 17706, 17707) c6( ) c7(beo, zQ, aW, Y, Am, aXY, aT, tv, wR, tu, ci, bQx, zP, fb, f); #c1(RPL30) c2(NP_000980) c3(60S ribosomal protein L30) c4(17894, 17895) c5(17896, 17897, 17898, 17899, 17900, 17901, 17902, 17903, 17904, 17905, 17906, 17907, 17908, 17909, 17910, 17911) c6( ) c7(bB, bE, T, jf, B); #c1(RPL31) c2(NP_000984) c3(60S ribosomal protein L31 isoform 1) c4(17953, 17954, 17955) c5(17956, 17957, 17958, 17959, 17960, 17961, 17962, 17963, 17964, 17965, 17966, 17967, 17968, 17969, 17970, 17971, 17972, 17973, 17974, 17975, 17976, 17977) c6( ) c7(aT); #c1(RPL31) c2(NP_001092047) c3(60S ribosomal protein L31 isoform 2) c4(39260, 39261) c5(39262, 39263, 39264, 39265, 39266, 39267) c6( ) c7(aT); #c1(RPL31) c2(NP_001093163) c3(60S ribosomal protein L31 isoform 3) c4(39906) c5(39907, 39908, 39909, 39910, 39911, 39912, 39913, 39914, 39915, 39916, 39917, 39918) c6( ) c7(aT); #c1(RPL34) c2(XP_006714350) c3(60S ribosomal protein L34 isoform X1) c4(287758) c5(287759, 287760, 287761, 287762, 287763, 287764, 287765, 287766, 287767, 287768, 287769, 287770, 287771, 287772, 287773, 287774, 287775, 287776, 287777, 287778, 287779, 287780) c6( ) c7(ck, iJ, bP); #c1(RPL35) c2(NP_009140) c3(60S ribosomal protein L35) c4(171055, 171056) c5(171057, 171058, 171059, 171060, 171061, 171062, 171063, 171064, 171065, 171066, 171067, 171068) c6( ) c7(aT); #c1(RPL36A) c2(NP_066357) c3(60S ribosomal protein L36a isoform a) c4(207184, 207185, 207186) c5(207187, 207188, 207189, 207190, 207191, 207192, 207193, 207194) c6( ) c7(ae, T, hd, d, nM, eM, nB, iO, aW, aZ, n, K, g, aDZ, bz, B, ad, Qt); #c1(RPL36AL) c2(NP_000992) c3(60S ribosomal protein L36a-like) c4( ) c5(18014, 18015, 18016, 18017, 18018, 18019, 18020, 18021, 18022, 18023) c6( ) c7(B); #c1(RPL37A) c2(NP_000989) c3(60S ribosomal protein L37a) c4(17978, 17979) c5(17980, 17981, 17982, 17983, 17984, 17985, 17986, 17987, 17988, 17989, 17990, 17991) c6( ) c7(kl); #c1(RPL38) c2(NP_000990) c3(60S ribosomal protein L38) c4(17992) c5(17993, 17994, 17995, 17996, 17997, 17998, 17999, 18000, 18001, 18002, 18003, 18004, 18005, 18006) c6( ) c7(aBU, aZR, zo); #c1(RPL39) c2(NP_000991) c3(60S ribosomal protein L39) c4(18007) c5(18008, 18009, 18010, 18011, 18012, 18013) c6( ) c7(aE); #c1(RPL39L) c2(NP_443201) c3(60S ribosomal protein L39-like) c4(229857) c5(229858, 229859, 229860, 229861, 229862, 229863, 229864, 229865, 229866, 229867, 229868, 229869, 229870, 229871) c6( ) c7(t, B, m); #c1(RPL4) c2(NP_000959) c3(60S ribosomal protein L4) c4(17708, 17709, 17710) c5(17711, 17712, 17713, 17714, 17715, 17716, 17717, 17718, 17719, 17720, 17721, 17722, 17723, 17724, 17725, 17726, 17727, 17728) c6( ) c7(az); #c1(RPL41) c2(NP_066927) c3(60S ribosomal protein L41) c4(207529) c5( ) c6( ) c7(cAl, eW); #c1(RPL6) c2(XP_006719612) c3(60S ribosomal protein L6 isoform X1) c4(292663, 292664, 292665, 292666, 292667) c5(292668, 292669, 292670, 292671, 292672, 292673, 292674, 292675, 292676, 292677, 292678, 292679, 292680, 292681, 292682, 292683, 292684, 292685, 292686, 292687, 292688, 292689, 292690, 292691, 292692, 292693, 292694, 292695, 292696, 292697, 292698, 292699, 292700, 292701, 292702, 292703, 292704, 292705) c6( ) c7(zQ, yS, aA, aF, ci, an, zP, fh); #c1(RPL7) c2(NP_000962) c3(60S ribosomal protein L7) c4(17729) c5(17730, 17731, 17732, 17733, 17734, 17735) c6( ) c7(cAm); #c1(RPL7A) c2(NP_000963) c3(60S ribosomal protein L7a) c4(17736, 17737, 17738) c5(17739, 17740, 17741, 17742, 17743, 17744, 17745, 17746, 17747, 17748, 17749, 17750, 17751, 17752, 17753, 17754, 17755, 17756) c6( ) c7(B, aW, eH, r, aT, K, eF, q); #c1(RPLP0) c2(NP_000993) c3(60S acidic ribosomal protein PO) c4(18024, 18025) c5(18026, 18027, 18028, 18029, 18030, 18031, 18032, 18033, 18034, 18035, 18036, 18037, 18038, 18039, 18040, 18041, 18042, 18043, 18044, 18045) c6( ) c7(ck, bM, eM, bE, r, fe, q, B); #c1(RPLP1) c2(NP_000994) c3(60S acidic ribosomal protein P1 isoform 1) c4(18046) c5(18047, 18048, 18049, 18050, 18051, 18052) c6( ) c7(T, jz, hb, bE, wL, cj, bB, y); #c1(RPLP1) c2(NP_998890) c3(60S acidic ribosomal protein P1 isoform 2) c4(266078) c5(266079, 266080, 266081, 266082, 266083, 266084) c6( ) c7(T, jz, hb, bE, wL, cj, bB, y); #c1(RPN1) c2(NP_002941) c3(dolichyl-diphosphooligosaccharide-protein glycosyltransferase subunit 1 precursor) c4(133762, 133763, 133764, 133765) c5(133766, 133767, 133768, 133769, 133770, 133771, 133772, 133773, 133774, 133775, 133776, 133777, 133778, 133779, 133780, 133781, 133782, 133783, 133784, 133785, 133786, 133787, 133788, 133789) c6( ) c7(ck, bs, j, bw, bt, br, ad); #c1(RPN2) c2(NP_001129243) c3(dolichyl-diphosphooligosaccharide-protein glycosyltransferase subunit 2 isoform 2 precursor) c4(51300) c5(51301, 51302, 51303, 51304, 51305, 51306, 51307, 51308, 51309, 51310, 51311, 51312, 51313, 51314, 51315, 51316, 51317, 51318) c6( ) c7(bL, T, dH, aW, ff, al, en, ad); #c1(RPN2) c2(NP_002942) c3(dolichyl-diphosphooligosaccharide-protein glycosyltransferase subunit 2 isoform 1 precursor) c4(133790) c5(133791, 133792, 133793, 133794, 133795, 133796) c6( ) c7(bL, T, dH, aW, ff, aT, en, ad); #c1(RPN2) c2(XP_005260548) c3(dolichyl-diphosphooligosaccharide-protein glycosyltransferase subunit 2 isoform X2) c4(276607, 276608) c5( ) c6( ) c7(bL, T, dH, aW, ff, al, en, ad); #c1(RPN2) c2(XP_006723914) c3(dolichyl-diphosphooligosaccharide-protein glycosyltransferase subunit 2 isoform X4) c4(295448) c5( ) c6( ) c7(bL, T, dH, aW, ff, al, en, ad); #c1(RPN2) c2(XP_006723915) c3(dolichyl-diphosphooligosaccharide-protein glycosyltransferase subunit 2 isoform X5) c4(295449, 295450, 295451, 295452) c5(295453, 295454, 295455, 295456, 295457, 295458, 295459, 295460, 295461, 295462, 295463, 295464, 295465, 295466, 295467, 295468, 295469, 295470, 295471, 295472, 295473, 295474, 295475, 295476, 295477, 295478, 295479, 295480, 295481, 295482) c6( ) c7(bL, T, dH, aW, ff, al, en, ad); #c1(RPP14) c2(NP_001092253) c3(ribonuclease P protein subunit p14) c4(39443) c5(39444, 39445, 39446, 39447, 39448, 39449, 39450, 39451, 39452, 39453, 39454, 39455, 39456, 39457, 39458) c6( ) c7(sc, jf, T, ft, aEj, aW, Y, jh, aT, cj, r, dL, aF, ci, K, q, EY, g, fr, B, ad); #c1(RPP21) c2(NP_001186049) c3(ribonuclease P protein subunit p21 isoform 1) c4(76935, 76936, 76937) c5(76938, 76939, 76940, 76941, 76942, 76943, 76944, 76945, 76946, 76947, 76948, 76949, 76950, 76951, 76952, 76953, 76954, 76955, 76956, 76957, 76958, 76959, 76960, 76961, 76962, 76963, 76964, 76965, 76966, 76967, 76968) c6( ) c7(eZ); #c1(RPP21) c2(NP_001186050) c3(ribonuclease P protein subunit p21 isoform 3) c4(76969, 76970) c5(76971, 76972, 76973, 76974, 76975, 76976, 76977, 76978, 76979, 76980, 76981, 76982, 76983, 76984, 76985, 76986, 76987, 76988, 76989, 76990, 76991, 76992, 76993, 76994, 76995, 76996, 76997, 76998, 76999) c6( ) c7(eZ); #c1(RPP21) c2(NP_079115) c3(ribonuclease P protein subunit p21 isoform 2) c4(217103, 217104, 217105) c5(217106, 217107, 217108, 217109, 217110, 217111, 217112, 217113, 217114, 217115, 217116, 217117, 217118, 217119, 217120, 217121, 217122, 217123, 217124, 217125, 217126, 217127, 217128, 217129, 217130, 217131, 217132, 217133, 217134, 217135, 217136, 217137, 217138, 217139, 217140, 217141, 217142, 217143, 217144, 217145, 217146, 217147, 217148, 217149, 217150, 217151, 217152, 217153, 217154, 217155, 217156, 217157, 217158, 217159, 217160, 217161, 217162, 217163, 217164, 217165, 217166, 217167) c6( ) c7(eZ); #c1(RPP25) c2(NP_060263) c3(ribonuclease P protein subunit p25) c4(195116) c5(195117, 195118, 195119, 195120, 195121, 195122, 195123, 195124, 195125, 195126) c6( ) c7(hb, bN); #c1(RPP38) c2(NP_001252530) c3(ribonuclease P protein subunit p38) c4(94174, 94175, 94176) c5(94177, 94178, 94179, 94180, 94181, 94182, 94183, 94184, 94185, 94186, 94187, 94188, 94189, 94190, 94191, 94192, 94193, 94194, 94195, 94196, 94197, 94198, 94199, 94200, 94201, 94202) c6( ) c7(Y); #c1(RPP40) c2(NP_006629) c3(ribonuclease P protein subunit p40 isoform a) c4(166723, 166724) c5(166725, 166726, 166727, 166728, 166729, 166730, 166731, 166732, 166733, 166734, 166735, 166736) c6( ) c7(aw); #c1(RPP40) c2(XP_011512555) c3(ribonuclease P protein subunit p40 isoform X1) c4( ) c5(303428) c6( ) c7(aw); #c1(RPRD1A) c2(NP_001290340) c3(regulation of nuclear pre-mRNA domain-containing protein 1A isoform 2) c4(115206) c5(115207, 115208, 115209, 115210, 115211, 115212) c6( ) c7(ey); #c1(RPRD1A) c2(NP_001290342) c3(regulation of nuclear pre-mRNA domain-containing protein 1A isoform 3) c4(115213) c5(115214, 115215, 115216, 115217, 115218, 115219) c6( ) c7(ey); #c1(RPRD1A) c2(NP_060640) c3(regulation of nuclear pre-mRNA domain-containing protein (A isoform 1) c4(196941) c5(196942, 196943, 196944, 196945, 196946, 196947, 196948, 196949, 196950, 196951) c6( ) c7(ey); #c1(RPRD1B) c2(NP_067038) c3(regulation of nuclear pre-mRNA domain-containing protein 1B) c4(208566) c5(208567, 208568, 208569, 208570, 208571, 208572) c6( ) c7(bK); #c1(RPRD2) c2(NP_001284602) c3(regulation of nuclear pre-mRNA domain-containing protein 2 isoform 2) c4(112145) c5(112146, 112147, 112148, 112149, 112150, 112151) c6( ) c7(cm); #c1(RPRD2) c2(NP_001284603) c3(regulation of nuclear pre-mRNA domain-containing protein 2 isoform 3) c4(112152) c5(112153, 112154, 112155, 112156, 112157, 112158) c6( ) c7(cm); #c1(RPRM) c2(NP_062819) c3(protein reprimo) c4(202146) c5(202147, 202148, 202149, 202150, 202151, 202152) c6( ) c7(ht, akb, aA, EQ, r); #c1(RPS10) c2(NP_001191020) c3(40S ribosomal protein S10) c4(80549) c5(80550, 80551, 80552, 80553, 80554, 80555) c6( ) c7(cAn, CJ, B, vi); #c1(RPS14) c2(NP_001020242) c3(40S ribosomal protein S14) c4(28333) c5(28334, 28335, 28336, 28337, 28338, 28339, 28340, 28341, 28342, 28343, 28344, 28345, 28346, 28347, 28348, 28349, 28350, 28351) c6( ) c7(Gq, zQ, CJ, Js, aW, oW, aT, kD, Xg, bw, v, oX, ci, br, zP, Jo); #c1(RPSI5A) c2(NP_001025180) c3(40S ribosomal protein S15a) c4(29452, 29453) c5(29454, 29455, 29456, 29457, 29458, 29459, 29460, 29461, 29462, 29463) c6( ) c7(B, vi); #c1(RPS16) c2(XP_005259194) c3(40S ribosomal protein S16 isoform X1) c4(275974) c5(275975, 275976, 275977, 275978, 275979, 275980) c6( ) c7(aC, CJ); #c1(RPS18) c2(NP_072045) c3(40S ribosomal protein S18) c4(212450, 212451, 212452) c5(212453, 212454, 212455, 212456, 212457, 212458, 212459, 212460, 212461, 212462, 212463, 212464, 212465, 212466, 212467, 212468, 212469, 212470, 212471, 212472, 212473, 212474, 212475, 212476, 212477, 212478, 212479, 212480, 212481, 212482, 212483, 212484, 212485, 212486, 212487, 212488, 212489, 212490, 212491, 212492, 212493, 212494, 212495, 212496) c6( ) c7(bB, bE, eZ); #c1(RPS19BP1) c2(NP_919307) c3(active regulator of SIRT1) c4(258532) c5(258533, 258534, 258535, 258536, 258537, 258538) c6( ) c7(T, arh); #c1(RPS2) c2(NP_002943) c3(40S ribosomal protein S2) c4(133797, 133798, 133799, 133800) c5(133801, 133802, 133803, 133804, 133805, 133806, 133807, 133808, 133809, 133810, 133811, 133812, 133813, 133814, 133815, 133816, 133817, 133818, 133819, 133820, 133821, 133822, 133823, 133824) c6( ) c7(T, dA, MD, aeB, vJ, ae, ad); #c1(RPS20) c2(NP_001139699) c3(40S ribosomal protein S20 isoform 1) c4(59760, 59761) c5(59762, 59763, 59764, 59765, 59766, 59767) c6( ) c7(bB, bE, jf, q, bh); #c1(RPS24) c2(NP_001135754) c3(40S ribosomal protein S24 isoform b) c4( ) c5(53087, 53088, 53089, 53090, 53091, 53092) c6( ) c7(aov, CJ, Gp, aw, EQ, cAo, qm); #c1(RPS24) c2(NP_001135755) c3(40S ribosomal protein S24 isoform e) c4( ) c5(53093, 53094, 53095, 53096, 53097, 53098) c6( ) c7(aov, CJ, Gp, aw, EQ, cAo, qm); #c1(RPS24) c2(NP_001135757) c3(40S ribosomal protein S24 isoform d) c4(53099, 53100, 53101, 53102) c5(53103, 53104, 53105, 53106, 53107, 53108) c6( ) c7(aov, CJ, Gp, aw, EQ, cAo, qm); #c1(RPS24) c2(NP_148982) c3(40S ribosomal protein S24 isoform a) c4( ) c5(226698, 226699, 226700, 226701, 226702, 226703) c6( ) c7(aov, CJ, Gp, aw, EQ, cAo, qm); #c1(RPS27) c2(NP_001021) c3(40S ribosomal protein S27) c4(29157, 29158) c5( ) c6( ) c7(T, aaH, aA, aLl, lE, hb, aT, bNj, af, d, B, aW, Y, dq, kp, aF, iK, kg, Lq, ati, wS, vi); #c1(RPS27A) c2(NP_001170884) c3(ubiquitin-40S ribosomal protein S27a precursor) c4(69568, 69569, 69570) c5(69571, 69572, 69573, 69574, 69575, 69576, 69577, 69578, 69579, 69580, 69581, 69582, 69583, 69584, 69585, 69586, 69587, 69588, 69589, 69590) c6( ) c7(rR, bE, aW, wu, jh, aT, dL, bK, K, wm, XM, eL, JO, ad); #c1(RPS27L) c2(NP_057004) c3(40S ribosomal protein S27-like) c4(188423, 188424, 188425, 188426, 188427, 188428) c5(188429, 188430, 188431, 188432, 188433, 188434, 188435, 188436, 188437, 188438, 188439, 188440, 188441, 188442, 188443, 188444, 188445, 188446, 188447, 188448, 188449, 188450, 188451) c6( ) c7(q, r); #c1(RPS29) c2(NP_001023) c3(40S ribosomal protein S29 isoform 1) c4(29159, 29160, 29161, 29162, 29163) c5(29164, 29165, 29166, 29167, 29168, 29169) c6( ) c7(av); #c1(RPS29) c2(NP_001025172) c3(40S ribosomal protein S29 isoform 2) c4(29391) c5(29392, 29393, 29394, 29395, 29396, 29397) c6( ) c7(av); #c1(RPS3) c2(NP_000996) c3(40S ribosomal protein S3 isoform 1) c4(18053) c5(18054, 18055, 18056, 18057, 18058, 18059, 18060, 18061, 18062, 18063, 18064, 18065, 18066, 18067) c6( ) c7(Y, ns, ff, caU, cy, en); #c1(RPS3) c2(NP_001247435) c3(40S ribosomal protein S3 isoform 2) c4(93616) c5(93617, 93618, 93619, 93620, 93621, 93622) c6( ) c7(Y, ns, ff, caU, cy, en); #c1(RPS3A) c2(NP_000997) c3(40S ribosomal protein S3a isoform 1) c4(18068, 18069, 18070, 18071, 18072) c5(18073, 18074, 18075, 18076, 18077, 18078, 18079, 18080, 18081, 18082, 18083, 18084, 18085, 18086, 18087, 18088, 18089, 18090, 18091, 18092, 18093, 18094, 18095, 18096, 18097, 18098, 18099, 18100, 18101, 18102, 18103, 18104, 18105, 18106) c6( ) c7(fK, av, B, K, af); #c1(RPS4X) c2(NP_000998) c3(40S ribosomal protein S4, X isoform X isoform) c4(18107, 18108) c5(18109, 18110, 18111, 18112, 18113, 18114, 18115, 18116, 18117, 18118, 18119, 18120) c6( ) c7(xS, T, aiV, aeB, aW, azq, vJ, r, aT, wR, y, q); #c1(RPS4Y1) c2(NP_000999) c3(40S ribosomal protein S4, Y isoform 1) c4(18121, 18122) c5(18123, 18124, 18125, 18126, 18127, 18128, 18129, 18130, 18131, 18132, 18133, 18134) c6( ) c7(vJ); #c1(RPS6KA2) c2(NP_001006933) c3(ribosomal protein S6 kinase alpha-2 isoform b) c4(22352) c5(22353, 22354, 22355, 22356, 22357, 22358) c6( ) c7(bv, T, aE, bE, aW, NS, Y, ff, NT, aT, bB, cd, lo, aj, ci, m, en, t, ae, X, ad); #c1(RPS6KA2) c2(NP_066958) c3(ribosomal protein S6 kinase alpha-2 isoform a) c4(207753, 207754) c5(207755, 207756, 207757, 207758, 207759, 207760, 207761, 207762) c6( ) c7(bv, T, aE, bE, aW, NS, Y, ff, NT, aT, bB, cd, lo, aj, ci, m, en, t, ae, X, ad); #c1(RPS6KA2) c2(XP_005267149) c3(ribosomal protein S6 kinase alpha-2 isoform X4) c4(280791, 280792) c5(280793, 280794, 280795, 280796, 280797, 280798, 280799, 280800, 280801, 280802, 280803, 280804) c6( ) c7(bv, T, aE, bE, aW, NS, Y, ff, NT, aT, bB, cd, lo, aj, ci, m, en, t, ae, X, ad); #c1(RPS6KA2) c2(XP_011534361) c3(ribosomal protein S6 kinase alpha-2 isoform X1) c4(330738) c5( ) c6( ) c7(bv, T, aE, bE, aW, NS, Y, ff, NT, aT, bB, cd, lo, aj, ci, m, en, t, ae, X, ad); #c1(RPS6KA2) c2(XP_011534363) c3(ribosomal protein S6 kinase alpha-2 isoform X5) c4( ) c5(330739, 330740, 330741, 330742, 330743, 330744, 330745, 330746, 330747, 330748, 330749, 330750, 330751, 330752) c6( ) c7(bv, T, aE, bE, aW, NS, Y, ff, NT, aT, bB, cd, lo, aj, ci, m, en, t, ae, X, ad); #c1(RPS6KA3) c2(NP_004577) c3(ribosomal protein S6 kinase alpha-3) c4(147850, 147851) c5(147852, 147853, 147854, 147855, 147856, 147857, 147858) c6( ) c7(bL, aoU, T, fd, bB, hy, v, ci, nA, ad, yt, cAp, nd, KH, aT, h, dz, K, cx, nJ, ae, aIK, B, BN, et, aW, Y, ff, eH, nf, gl, lo, aVW, en, eF, t, cm, fq, ik, ck, kP, iT, SE, bE, m, bC, fh, ne); #c1(RPS6KA3) c2(XP_005274630) c3(ribosomal protein S6 kinase alpha-3 isoform X3) c4(285252) c5(285253, 285254) c6( ) c7(bL, aoU, T, fd, bB, hy, v, ci, nA, ad, yt, cAp, nd, kH, aT, h, dz, K, cx, nJ, ae, alk, B, BN, et, aW, Y, ff, eH, nf, gl, lo, aVW, en, eF, t, cm, fq, ik, ck, kP, iT, SE, bE, m, bC, fh, ne); #c1(RPS6KA3) c2(XP_005274634) c3(ribosomal protein S6 kinase alpha-3 isoform X8) c4(285255, 285256) c5(285257, 285258, 285259, 285260, 285261, 285262, 285263, 285264, 285265, 285266, 285267, 285268) c6( ) c7(bL, aoU, T, fd, bB, hy, v, ci, nA, ad, yt, cAp, nd, kH, aT, h, dz, K, cx, nJ, ae, alk, B, BN, et, aW, Y, ff, eH, nf, gl, lo, aVW, en, eF, t, cm, fq, ik, ck, kP, iT, SE, bE, m, bC, fh, ne); #c1(RPS6KA3) c2(XP_006724570) c3(ribosomal protein S6 kinase alpha-3 isoform X7) c4( ) c5(296077, 296078, 296079, 296080, 296081, 296082, 296083, 296084, 296085, 296086) c6( ) c7(bL, aoU, T, fd, bB, hy, v, ci, nA, ad, yt, cAp, nd, kH, aT, h, dz, K, cx, nJ, ae, alk, B, BN, et, aW, Y, ff, eH, nf, gl, lo, aVW, en, eF, t, cm, fq, iK, ck, kP, lT, SE, bE, m, bC, fh, ne); #c1(RPS6KA3) c2(XP_011543865) c3(ribosomal protein S6 kinase alpha-3 isoform X6) c4(342994) c5(342995, 342996, 342997, 342998, 342999) c6( ) c7(bL, aoU, T, fd, bB, hy, v, ci, nA, ad, yt, cAp, nd, KH, aT, h, dz, K, cx, nJ, ae, alk, B, BN, et, aW, Y, ff, eH, nf, gl, lo, aVW, en, eF, t, cm, fq, ik, ck, kP, iT, SE, bE, m, bC, fh, ne); #c1(RPS6KA4) c2(NP_001006945) c3(ribosomal protein S6 kinase alpha-4 isoform b) c4(22372, 22373) c5(22374, 22375, 22376, 22377, 22378, 22379) c6( ) c7(y, AW, bc); #c1(RPS6KA4) c2(NP_001287731) c3(ribosomal protein S6 kinase alpha-4 isoform c) c4(112690) c5(112691, 112692, 112693, 112694, 112695, 112696, 112697, 112698, 112699, 112700, 112701, 112702) c6( ) c7(y, AW, bc); #c1(RPS6KA4) c2(NP_003933) c3(ribosomal protein SG kinase alpha-4 isoform a) c4(142289) c5(142290, 142291, 142292, 142293, 142294, 142295) c6( ) c7(y, AW, bc); #c1(RPS6KA5) c2(NP_004746) c3(ribosomal protein S6 kinase alpha-5 isoform a) c4(149225, 149226) c5(149227, 149228, 149229, 149230, 149231, 149232, 149233, 149234, 149235, 149236, 149237, 149238) c6( ) c7(bE, aW, ff, cr, aT, cu, dL, ci, K, en, y); #c1(RPS6KA5) c2(NP_872198) c3(ribosomal protein S6 kinase alpha-5 isoform b) c4(255976) c5(255977, 255978, 255979, 255980, 255981, 255982) c6( ) c7(bE, aW, ff, cr, aT, cu, dL, ci, K, en, y); #c1(RPS6KA6) c2(NP_055311) c3(ribosomal protein S6 kinase alpha-6) c4(180395) c5(180396, 180397, 180398, 180399, 180400, 180401) c6( ) c7(iQ, s, iA, bE, Y, nd, hl, aT, nf, h, fr, ci, K, fu, cI); #c1(RPS6KA6) c2(XP_011529220) c3(ribosomal protein S6 kinase alpha-6 isoform X1) c4(324672) c5(324673, 324674, 324675, 324676, 324677, 324678) c6( ) c7(iQ, s, iA, bE, Y, nd, hL, aT, nf, h, fr, ci, K, fu, cI); #c1(RPS6KB1) c2(NP_001258971) c3(ribosomal protein S6 kinase beta-1 isoform b) c4(96848) c5(96849, 96850, 96851, 96852, 96853, 96854) c6( ) c7(bL, T, hl, id, pf, aw, ht, cz, bB, n, hy, v, ci, V, eA, ad, ip, av, aE, NS, aT, iv, jy, dL, bl, K, qM, hu, ae, af, bc, eL, d, bux, BN, B, iU, aW, aq, yq, Ku, t, rY, kP, eM, bP, bE, X, mx, mg, mf, m, ne); #c1(RPS6KB1) c2(NP_001258972) c3(ribosomal protein S6 kinase beta-1 isoform c) c4(96855) c5(96856, 96857, 96858, 96859, 96860, 96861) c6( ) c7(bL, T, hl, id, pf, aw, ht, cz, bB, n, hy, v, ci, V, eA, ad, ip, av, aE, NS, aT, iv, jy, dL, bl, K, qM, hu, ae, af, bc, eL, d, bux, BN, B, iU, aW, aq, yq, Ku, t, rY, kP, eM, bP, bE, X, mx, mg, mf, m, ne); #c1(RPS6KB1) c2(NP_001258973) c3(ribosomal protein S6 kinase beta-1 isoform d) c4( ) c5(96862, 96863, 96864, 96865, 96866) c6( ) c7(bL, T, hl, id, pf, aw, ht, cz, bB, n, hy, v, ci, V, eA, ad, ip, av, aE, NS, aT, iv, jy, dL, bl, K, qM, hu, ae, af, bc, eL, d, bux, BN, B, iU, aW, aq, yq, Ku, t, rY, kP, eM, bP, bE, X, mx, mg, mf, m, ne); #c1(RPS6KB1) c2(NP_001258989) c3 (ribosomal protein S6 kinase beta-1 isoform e) c4(96900) c5( ) c6( ) c7(bL, T, hl, id, pf, aw, ht, cz, bB, n, hy, v, ci, V, eA, ad, ip, av, aE, NS, aT, iv, jy, dL, bl, K, qM, hu, ae, af, bc, eL, d, bux, BN, B, iU, aW, aq, yq, Ku, t, rY, kP, eM, bP, bE, X, mx, mg, mf, m, ne); #c1 (RPS6KB1) c2(NP_003152) c3(ribosomal protein S6 kinase beta-1 isoform a) c4(135730) c5(135731, 135732, 135733, 135734, 135735, 135736, 135737) c6( ) c7(bL, T, hl, id, pf, aw, ht, cz, bB, n, hy, v, ci, V, eA, ad, ip, av, aE, NS, aT, iv, jy, dL, bl, K, qM, hu, ae, af, bc, eL, d, bux, BN, B, iU, aW, aq, yq, Ku, t, rY, kP, eM, bP, bE, X, mx, mg, mf, m, ne); #c1(RPS6KB2) c2(NP_003943) c3(ribosomal protein S6 kinase beta-2) c4(142353, 142354) c5(142355, 142356, 142357, 142358, 142359, 142360, 142361, 142362, 142363, 142364, 142365, 142366, 142367, 142368, 142369) c6( ) c7(T, BN, eM, ne, aW, NS, aT, hy, np, X); #c1(RPS6KB2) c2(XP_005274221) c3(ribosomal protein S6 kinase beta-2 isoform X1) c4( ) c5(284995) c6( ) c7(T, BN, eM, ne, aW, NS, aT, hy, np, X); #c1(RPS9) c2(XP_006726028) c3(40S ribosomal protein S9 isoform X1) c4(297039, 297040, 297041) c5(297042, 297043, 297044, 297045, 297046, 297047, 297048, 297049, 297050, 297051, 297052, 297053, 297054, 297055, 297056, 297057, 297058, 297059, 297060, 297061, 297062, 297063, 297064, 297065, 297066, 297067, 297068, 297069, 297070, 297071, 297072, 297073, 297074, 297075, 297076, 297077, 297078) c6( ) c7(T, K); #c1(RPSA) c2(NP_002286) c3(40S ribosomal protein SA isoform 1) c4(127329, 127330, 127331) c5(127332, 127333, 127334, 127335, 127336, 127337, 127338, 127339, 127340, 127341, 127342, 127343, 127344, 127345, 127346, 127347, 127348, 127349, 127350, 127351, 127352) c6( ) c7(lD, bM, cZ, T, aw, jh, aA, bB, Hn, fd, ckj, j, ci, C, hF, V, q, BY, x, av, iu, ft, jz, aE, xc, yt, dV, kH, cU, aT, By, cy, h, K, dd, dQ, af, A, je, aW, cAq, Y, afS, cr, r, eZ, fe, aF, g, cl, eE, t, cm, bz, QA, ck, es, iM, eM, Jr, bE, iA, z, U, Zn, eo, k, m, afT, y, bt, X); #c1(RPTOR) c2(NP_001156506) c3(regulatory-associated protein of mTOR isoform 2) c4(61738, 61739) c5(61740, 61741, 61742, 61743, 61744, 61745, 61746, 61747, 61748, 61749, 61750, 61751) c6( ) c7(hl, zw, fR, oe, cr, fd, h, lo, kC, it); #c1(RPTOR) c2(NP_065812) c3(regulatory-associated protein of mTOR isoform 1) c4(205664) c5(205665, 205666, 205667, 205668, 205669, 205670) c6( ) c7(hl, zw, fR, oe, cr, fd, h, lo, kC, it); #c1(RQCD1) c2(NP_001258563) c3(cell differentiation protein RCD1 homolog isoform 1) c4(96132) c5(96133, 96134, 96135, 96136, 96137, 96138) c6( ) c7(aW, nw, bu, aT); #c1(RQCD1) c2(NP_001258564) c3(cell differentiation protein RCD1 homolog isoform 3) c4(96139) c5(96140, 96141, 96142, 96143, 96144, 96145) c6( ) c7(aW, nw, bu, aT); #c1(RACD1) c2(NP_005435) c3(cell differentiation protein RCD1 homolog isoform 2) c4(156151) c5(156152, 156153, 156154, 156155, 156156, 156157, 156158, 156159, 156160, 156161) c6( ) c7(aW, nw, bu, aT); #c1(RRAD) c2(NP_001122322) c3(GTP-binding protein RAD) c4(46528) c5(46529, 46530, 46531, 46532, 46533, 46534, 46535, 46536, 46537, 46538) c6( ) c7(bL, T, bP, c, eM, aW, aT, gZ, cy, aj, np, K, m, g, bl, t, ae, J, ad); #c1(RRAGA) c2(NP_006561) c3(ras-related GTP-binding protein A) c4(166236) c5(166237, 166238, 166239, 166240, 166241, 166242) c6( ) c7(apY, aiu, T, oy, akC); #c1(RRAS) c2(NP_006261) c3(ras-related protein R-Ras precursor) c4(163358) c5(163359, 163360, 163361, 163362, 163363, 163364) c6( ) c7(T, eG, bP, Pg, U, aT, auT, alR, alQ, K, alP, ae, ad, dR); #c1(RRAS2) c2(NP_001096139) c3(ras-related protein R-Ras2 isoform b) c4(41136) c5(41137, 41138, 41139, 41140, 41141, 41142, 41143, 41144, 41145, 41146, 41147, 41148, 41149, 41150, 41151, 41152, 41153, 41154, 41155, 41156, 41157, 41158) c6( ) c7(amg, jp, hW, aW, yk, jh, aT, ci, m, an, t, fq, B); #c1(RRAS2) c2(NP_001170785) c3(ras-related protein R-Ras2 isoform c) c4(69526) c5(69527, 69528, 69529, 69530, 69531, 69532, 69533, 69534, 69535) c6( ) c7(amg, jp, hW, aW, yk, jh, aT, ci, m, an, t, fq, B); #c1(RRAS2) c2(NP_036382) c3(ras-related protein R-Ras2 isoform a) c4(173432, 173433, 173434) c5(173435, 173436, 173437, 173438, 173439, 173440, 173441, 173442, 173443, 173444, 173445, 173446, 173447, 173448, 173449) c6( ) c7(amg, jp, hW, aW, yk, jh, aT, ci, m, an, t, fq, B); #c1(RRBP1) c2(NP_001036041) c3(ribosome-binding protein 1) c4(33814, 33815, 33816, 33817) c5(33818, 33819, 33820, 33821, 33822, 33823, 33824, 33825, 33826, 33827, 33828, 33829, 33830, 33831, 33832, 33833, 33834, 33835, 33836, 33837, 33838, 33839, 33840, 33841, 33842, 33843, 33844, 33845, 33846, 33847, 33848, 33849) c6( ) c7(n, T, oy, jh, akC); #c1(RREB1) c2(NP_001003700) c3(ras-responsive element-binding protein 1 isoform 3) c4(19982) c5(19983, 19984, 19985, 19986, 19987, 19988) c6( ) c7(T, dA, WT, ci, PJ, q, ad, eW, hX, hd, aE, nv, h, BZ, dL, K, an, ae, eL, avQ, ak, anR, d, Y, CT, lo, g, as, pb, ck, Qi, iO, vW, W, c, iT, eB, U, bC, bt, xg, B); #c1(RREB1) c2(NP_001161816) c3(ras-responsive element-binding protein 1 isoform 2) c4(66076, 66077, 66078) c5(66079, 66080, 66081, 66082, 66083, 66084, 66085, 66086, 66087, 66088, 66089, 66090) c6( ) c7(T, dA, WT, ci, PJ, q, ad, eW, hX, hd, aE, nv, h, BZ, dL, K, an, ae, eL, avQ, ak, anR, d, Y, CT, lo, g, as, pb, ck, di, iO, vW, W, c, iT, eB, U, bC, bt, xg, B); #c1(RREB1) c2(XP_006715221) c3(ras-responsive element-binding protein 1 isoform X1) c4(288840, 288841, 288842) c5(288843, 288844, 288845, 288846, 288847, 288848) c6( ) c7(T, dA, WT, ci, PJ, q, ad, eW, hX, hd, aE, nv, h, BZ, dL, K, an, ae, eL, avQ, ak, anR, d, Y, CT, lo, g, as, pb, ck, Qi, iO, vW, W, c, iT, eB, U, bC, bt, xg, B); #c1(RREB1) c2(XP_011513097) c3(ras-responsive element-binding protein 1 isoform X3) c4( ) c5(304802, 304803, 304804, 304805, 304806, 304807, 304808, 304809, 304810, 304811, 304812, 304813, 304814, 304815, 304816, 304817, 304818, 304819, 304820, 304821, 304822, 304823, 304824, 304825) c6( ) c7(T, dA, WT, ci, PJ, q, ad, eW, hX, hd, aE, nv, h, BZ, dL, K, an, ae, eL, avQ, ak, anR, d, Y, CT, lo, g, as, pb, ck, Qi, iO, vW, W, c, iT, eB, U, bC, bt, xg, B); #c1(RRH) c2(NP_006574) c3(visual pigment-like receptor peropsin) c4(166329) c5(166330, 166331, 166332, 166333, 166334, 166335) c6( ) c7(nC, nw); #c1(RRM1) c2(NP_001024) c3(ribonucleoside-diphosphate reductase large subunit) c4(29170, 29171, 29172) c5(29173, 29174, 29175, 29176, 29177, 29178, 29179) c6( ) c7(gV, T, aC, eR, jp, atC, aA, bB, n, hy, ci, bX, lu, q, er, av, aE, aT, iv, B, BN, hG, aW, ff, alR, apk, r, nY, iw, lo, aF, en, g, t, bz, iO, Qy, eM, bE, PV, at, bt, ne, hS); #c1(RRM2) c2(NP_001025) c3(ribonucleoside-diphosphate reductase subunit M2 isoform 2) c4(29625) c5( ) c6( ) c7(T, id, if, aA, n, hy, Vd, qN, lu, q, ad, eJ, iu, aE, WY, aT, dL, ey, ae, eL, kl, qt, aW, Y, r, aaE, kp, aF, g, mo, t, iK, bw, c, eM, gX, U, hL, m, EY, hE, bt, B); #c1(RRM2) c2(NP_001159403) c3(ribonucleoside-diphosphate reductase subunit M2 isoform 1) c4(63963) c5(63964, 63965, 63966, 63967, 63968, 63969) c6( ) c7(T, id, if, aA, n, hy, Vd, qN, lu, q, ad, eJ, iu, aE, WY, aT, dL, ey, ae, eL, kl, qt, aW, Y, r, aaE, kp, aF, g, mo, t, iK, bw, c, eM, gX, U, hL, m, EY, hE, bt, B); #c1(RRM2B) c2(NP_00165948) c3(ribonucleoside-diphosphate reductase subunit M2 B isoform 2) c4(68030, 68031) c5(68032) c6( ) c7(T, cAr, bB, hy, ci, aoz, q, ad, cAs, hb, aT, K, awL, cAt, ae, BN, aW, Y, ev, ff, r, GA, aF, en, g, iV, kC, akQ, SE, bE, it, ne); #c1(RRM2B) c2(NP_001165949) c3(ribonucleoside-diphosphate reductase subunit M2 B isoform 3) c4(68033) c5(68034, 68035, 68036, 68037, 68038, 68039, 68040, 68041, 68042, 68043, 68044, 68045, 68046, 68047, 68048, 68049, 68050, 68051, 68052) c6( ) c7(T, cAr, bB, hy, ci, aoz, q, ad, cAs, hb, aT, K, awL, cAt, ae, BN, aW, Y, ev, ff, r, GA, aF, en, g, iV, kC, akQ, SE, bE, it, ne); #c1(RRM2B) c2(NP_056528) c3(ribonucleoside-diphosphate reductase subunit M2 B isoform 1) c4(187938, 187939, 187940) c5(187941, 187942, 187943, 187944, 187945, 187946, 187947, 187948, 187949, 187950, 187951, 187952, 187953, 187954, 187955, 187956) c6( ) c7(T, cAr, bB, hy, ci, aoz, q, ad, cAs, hb, aT, K, awL, cAt, ae, BN, aW, Y, ev, ff, r, GA, aF, en, g, iV, kC, akQ, SE, bE, it, ne); #c1(RRN3) c2(NP_001287993) c3(RNA polymerase I-specific transcription initiation factor RRN3 isoform 2) c4(113311) c5(113312, 113313, 113314, 113315, 113316, 113317, 113318, 113319, 113320, 113321, 113322, 113323, 113324, 113325, 113326, 113327, 113328) c6( ) c7(Da); #c1(RRN3) c2(NP_060897) c3(RNA polymerase I-specific transcription initiation factor RRN3 isoform 1) c4(198460, 198461, 198462, 198463, 198464, 198465) c5(198466, 198467, 198468, 198469, 198470, 198471, 198472, 198473, 198474, 198475, 198476, 198477, 198478, 198479, 198480, 198481, 198482, 198483, 198484, 198485, 198486, 198487, 198488, 198489, 198490) c6( ) c7(Da); #c1(RRNAD1) c2(NP_001136032) c3(protein RRNAD1 isoform 2) c4(54090) c5(54091, 54092, 54093, 54094, 54095, 54096) c6( ) c7(af); #c1(RRNAD1) c2(NP_057081) c3(protein RRNAD1 isoform 1) c4(188855) c5(188856, 188857, 188858, 188859, 188860, 188861) c6( ) c7(af); #c1(RRP1) c2(NP_003674) c3(ribosomal RNA processing protein 1 homolog A) c4(140102, 140103) c5(140104, 140105, 140106, 140107, 140108, 140109, 140110, 140111, 140112, 140113, 140114, 140115) c6( ) c7(T, af); #c1(RRP1B) c2(NP_055871) c3(ribosomal RNA processing protein 1 homolog B) c4(184047) c5(184048, 184049, 184050, 184051, 184052, 184053) c6( ) c7(aW, T, aT); #c1(RRP9) c2(NP_004695) c3(U3 small nucleolar RNA-interacting protein 2) c4(148870) c5(148871, 148872, 148873, 148874, 148875, 148876) c6( ) c7(aA); #c1(RRS1) c2(NP_055984) c3(ribosome biogenesis regulatory protein homolog) c4(184884) c5(184885, 184886, 184887, 184888, 184889, 184890) c6( ) c7(T, yi, aW, aT, v, DG, ae, ad); #c1(RS1) c2(NP_000321) c3(retinoschisin precursor) c4(7028) c5(7029, 7030, 7031, 7032, 7033, 7034, 7035, 7036, 7037, 7038, 7039, 7040, 7041, 7042, 7043, 7044, 7045, 7046, 7047, 7048, 7049, 7050, 7051, 7052, 7053, 7054, 7055, 7056, 7057, 7058, 7059, 7060, 7061, 7062, 7063, 7064, 7065, 7066, 7067, 7068, 7069, 7070, 7071, 7072, 7073, 7074, 7075, 7076, 7077, 7078, 7079, 7080, 7081, 7082, 7083, 7084, 7085, 7086, 7087, 7088, 7089, 7090, 7091, 7092, 7093, 7094, 7095, 7096, 7097, 7098, 7099, 7100, 7101, 7102, 7103, 7104, 7105, 7106, 7107, 7108, 7109, 7110, 7111, 7112, 7113, 7114, 7115, 7116, 7117, 7118, 7119, 7120) c6( ) c7(W, nb, lR, afA, vy, cAu, nw, bgv); #c1(RSAD2) c2(XP_011508716) c3(radical S-adenosyl methionine domain-containing protein 2 isoform X1) c4(299326, 299327) c5(299328, 299329, 299330, 299331, 299332, 299333) c6( ) c7(dD, ds, Uq, aiM, j, eq, t, ai); #c1(RSAD2) c2(XP_011508717) c3(radical S-adenosyl methionine domain-containing protein 2 isoform X2) c4( ) c5(299334, 299335, 299336, 299337, 299338, 299339) c6( ) c7(dD, ds, Uq, aiM, j, eq, t, ai); #c1(RSF1) c2(NP_057662) c3(remodeling and spacing factor 1) c4(192178, 192179, 192180, 192181, 192182, 192183) c5(192184, 192185, 192186, 192187, 192188, 192189, 192190, 192191, 192192, 192193, 192194, 192195, 192196, 192197, 192198, 192199, 192200, 192201, 192202, 192203, 192204, 192205, 192206, 192207) c6( ) c7(XX, T, auY, id, bE, aW, ff, al, bB, n, agF, ci, m, en, t, bz, B, hX); #c1(RSF1) c2(XP_005274108) c3(remodeling and spacing factor 1 isoform X1) c4( ) c5(284934, 284935, 284936, 284937, 284938, 284939, 284940, 284941, 284942, 284943, 284944, 284945) c6( ) c7(XX, T, auY, id, bE, aW, ff, aT, bB, n, agF, ci, m, en, t, bz, B, hX); #c1(RSL1D1) c2(NP_056474) c3(ribosomal LI domain-containing protein 1) c4(187669, 187670, 187671) c5(187672, 187673, 187674, 187675, 187676, 187677, 187678, 187679, 187680, 187681, 187682, 187683, 187684, 187685, 187686, 187687, 187688, 187689, 187690, 187691, 187692, 187693) c6( ) c7(cAm); #c1(RSL24D1) c2(NP_057388) c3(probable ribosome biogenesis protein RLP24) c4(190513) c5(190514, 190515, 190516, 190517, 190518, 190519) c6( ) c7(oe); #c1(RSPH1) c2(NP_543136) c3(radial spoke head 1 homolog isoform 1) c4(233348) c5(233349, 233350, 233351, 233352, 233353, 233354) c6( ) c7(Pf, cAv, aw, MG, aMp); #c1(RSPH4A) c2(NP_001010892) c3(radial spoke head protein 4 homolog A isoform 1) c4(23982, 23983) c5(23984, 23985, 23986, 23987, 23988, 23989, 23990, 23991, 23992, 23993, 23994, 23995) c6( ) c7(cAw, hb, Pf, MG); #c1(RSPH4A) c2(NP_001155136) c3(radial spoke head protein 4 homolog A isoform 2) c4(61471, 61472) c5(61473, 61474, 61475, 61476, 61477, 61478, 61479, 61480, 61481, 61482, 61483, 61484) c6( ) c7(cAw, hb, Pf, MG); #c1(RSPH9) c2(NP_001180270) c3(radial spoke head protein 9 homolog isoform 2) c4(73661) c5(73662, 73663, 73664, 73665, 73666, 73667) c6( ) c7(cAx, BV, Pf, MG); #c1(RSPH9) c2(NP_689945) c3(radial spoke head protein 9 homolog isoform 1) c4(244442) c5(244443, 244444, 244445, 244446, 244447, 244448) c6( ) c7(cAx, BV, Pf, MG); #c1(RSPH9) c2(XP_011512658) c3(radial spoke head protein 9 homolog isoform X2) c4(303635, 303636) c5(303637, 303638, 303639, 303640, 303641, 303642, 303643, 303644, 303645, 303646, 303647, 303648) c6( ) c7(cAx, BV, Pf, MG); #c1(RSPO2) c2(NP_001269792) c3(R-spondin-2 isoform 2 precursor) c4(102826) c5(102827, 102828, 102829, 102830, 102831, 102832, 102833, 102834, 102835, 102836) c6( ) c7(y, hP, X, aT); #c1(RSPO2) c2(NP_848660) c3(R-spondin-2 isoform 1 precursor) c4(254125, 254126, 254127) c5(254128, 254129, 254130, 254131, 254132, 254133, 254134, 254135, 254136, 254137, 254138, 254139, 254140, 254141, 254142, 254143, 254144, 254145, 254146) c6( ) c7(y, hP, X, aT); #c1(RSPO2) c2(XP_011515320) c3(R-spondin-2 isoform X1) c4(308189, 308190, 308191) c5(308192, 308193) c6( ) c7(y, hP, X, aT); #c1(RSPO3) c2(NP_116173) c3(R-spondin-3 precursor) c4(225564, 225565) c5(225566, 225567, 225568, 225569, 225570, 225571, 225572, 225573, 225574, 225575, 225576, 225577, 225578, 225579, 225580, 225581) c6(225582, 225583, 225584, 225585) c7(aw, X, bA, aT); #c1(RSPO4) c2(NP_001025042) c3(R-spondin-4 isoform 1 precursor) c4(29260) c5(29261, 29262, 29263, 29264, 29265, 29266) c6( ) c7(byn, cAy); #c1(RSPO4) c2(NP_001035096) c3(R-spondin-4 isoform 2 precursor) c4(32503) c5(32504, 32505, 32506, 32507, 32508, 32509) c6( ) c7(byn, cAy); #c1(RSRC1) c2(NP_001258763) c3(serine/Arginine-related protein 53 isoform 2) c4(96511) c5(96512, 96513, 96514, 96515, 96516, 96517, 96518, 96519, 96520, 96521) c6( ) c7(cm, v); #c1(RSRC1) c2(NP_057709) c3(serine/Arginine-related protein 53 isoform 1) c4(192462, 192463, 192464) c5(192465, 192466, 192467, 192468, 192469, 192470, 192471, 192472, 192473, 192474, 192475, 192476, 192477, 192478, 192479, 192480, 192481, 192482, 192483, 192484, 192485, 192486) c6( ) c7(cm, v); #c1(RSRC1) c2(XP_011511178) c3(serine/Arginine-related protein 53 isoform X2) c4( ) c5(301653, 301654, 301655, 301656, 301657) c6( ) c7(cm, v); #c1(RSRC2) c2(NP_075388) c3(arginine/serine-rich coiled-coil protein 2) c4(213843) c5( ) c6( ) c7(T, it, ht, hu); #c1(RSRC2) c2(XP_005253659) c3(arginine/serine-rich coiled-coil protein 2 isoform X3) c4(272614) c5( ) c6( ) c7(T, it, ht, hu); #c1(RSRC2) c2(XP_011536990) c3(arginine/serine-rich coiled-coil protein 2 isoform X2) c4(334719) c5( ) c6( ) c7(T, it, ht, hu); #c1(RSRC2) c2(XP_011536991) c3(arginine/serine-rich coiled-coil protein 2 isoform X4) c4( ) c5(334720, 334721, 334722, 334723, 334724, 334725, 334726, 334727, 334728, 334729, 334730, 334731, 334732, 334733, 334734, 334735, 334736, 334737) c6( ) c7(T, it, ht, hu); #c1(RSRP1) c2(XP_011540100) c3(arginine/serine-rich protein 1 isoform X2) c4(338439) c5(338440, 338441, 338442, 338443, 338444, 338445, 338446, 338447, 338448, 338449, 338450, 338451, 338452) c6( ) c7(aw); #c1(RSU1) c2(NP_036557) c3(ras suppressor protein 1 isoform 1) c4(174372, 174373) c5(174374, 174375, 174376, 174377, 174378, 174379, 174380, 174381, 174382, 174383, 174384) c6( ) c7(bF, d, ac, aW, aT, Nl, eA, bA); #c1(RSU1) c2(NP_689937) c3(ras suppressor protein 1 isoform 2) c4( ) c5(244396, 244397, 244398, 244399, 244400) c6( ) c7(bF, d, ac, aW, aT, Nl, eA, bA); #c1(RTCB) c2(NP_055121) c3(tRNA-splicing ligase RtcB homolog) c4(179073, 179074) c5(179075, 179076, 179077, 179078, 179079, 179080, 179081, 179082, 179083, 179084, 179085, 179086) c6( ) c7(bz); #c1(RTEL1) c2(NP_001269938) c3(regulator of telomere elongation helicase 1 isoform 3) c4(103271, 103272) c5(103273, 103274, 103275, 103276, 103277, 103278, 103279, 103280, 103281, 103282, 103283, 103284, 103285) c6( ) c7(Kd, T, bM, fM, iW, jx, eX, qy, fd, jh, ci, apt, hF, eA, aoH, x, aoQ, F, aT, h, eV, bde, pk, aJu, sb, asp, BN, d, iU, aW, Y, mQ, CT, hv, lo, aoY, La, as, ho, lk, ck, ou, cT, rR, Zg, dB, rW, dc, km, cAz, cj, dW, oX, k, ij, zv, bt, jr); #c1(RTEL1) c2(NP_057518) c3(regulator of telomere elongation helicase 1 isoform 1) c4(191209) c5(191210, 191211, 191212, 191213, 191214, 191215) c6( ) c7(Kd, T, bM, fM, iW, jx, eX, qy, fd, jh, ci, apt, hF, eA, aoH, x, aoQ, F, aT, h, eV, bde, pk, aJu, sb, asp, BN, d, iU, aW, Y, mQ, CT, hv, lo, aoY, La, as, ho, lk, ck, ou, cT, rR, Zg, dB, rW, dc, km, cAz, cj, dW, oX, k, ij, zv, bt, jr); #c1(RTEL1) c2(NP_116575) c3(regulator of telomere elongation helicase 1 isoform 2) c4(226287) c5(226288, 226289, 226290, 226291, 226292, 226293) c6( ) c7(Kd, T, bM, fM, iW, jx, eX, qy, fd, jh, ci, apt, hF, eA, aoH, x, aoQ, F, aT, h, eV, bde, pk, aJu, sb, asp, BN, d, iU, aW, Y, mQ, CT, hv, lo, aoY, La, as, ho, lk, ck, ou, cT, rR, Zg, dB, rW, dc, km, cAz, cj, dW, oX, k, ij, zv, bt, jr); #c1(RTKN) c2(NP_001015055) c3(rhotekin isoform a) c4(26184, 26185) c5(26186, 26187, 26188, 26189, 26190, 26191, 26192) c6( ) c7(T, d, Jl, aW, aT, KO, ci, eL); #c1(RTKN) c2(NP_001015056) c3(rhotekin isoform c) c4( ) c5(26193, 26194, 26195, 26196, 26197) c6( ) c7(T, d, Jl, aW, aT, KO, ci, eL); #c1(RTKN) c2(NP_149035) c3(rhotekin isoform b) c4(226875) c5(226876, 226877, 226878, 226879, 226880, 226881) c6( ) c7(T, d, Jl, aW, aT, KO, ci, eL); #c1(RTKN2) c2(NP_001269870) c3(rhotekin-2 isoform 2) c4( ) c5(102990, 102991, 102992, 102993, 102994, 102995) c6( ) c7(y); #c1(RTKN2) c2(NP_660350) c3(rhotekin-2 isoform 1) c4(241047) c5(241048, 241049, 241050, 241051, 241052, 241053) c6( ) c7(y); #c1(RTKN2) c2(XP_006717758) c3(rhotekin-2 isoform X2) c4(291321) c5( ) c6( ) c7(y); #c1(RTKN2) c2(XP_011537761) c3(rhotekin-2 isoform X5) c4(335294, 335295) c5(335296, 335297, 335298, 335299, 335300, 335301, 335302, 335303, 335304, 335305, 335306, 335307) c6( ) c7(y); #c1(RTL1) c2(NP_001128360) c3(retrotransposon-like protein 1) c4(49963) c5(49964, 49965, 49966, 49967, 49968, 49969) c6( ) c7(bL, T, dA, qj, ci, qN, bNX, ds, Xh, cp, aFS, eG, kj, asf, Y, HA, lo, bu, kk, cm, iP, bm, pC); #c1(RTN1) c2(NP_066959) c3(reticulon-1 isoform A) c4(207763, 207764, 207765) c5(207766, 207767, 207768, 207769, 207770, 207771, 207772, 207773, 207774, 207775, 207776, 207777, 207778, 207779, 207780, 207781, 207782, 207783) c6( ) c7(aV, af, aW, ds, tT, anV, aT, j, fb, q, aa); #c1(RTN1) c2(NP_996734) c3(reticulon-1 isoform C) c4(264367) c5(264368, 264369, 264370, 264371, 264372, 264373) c6( ) c7(aV, af, aW, ds, tT, anV, al, j, fb, q, aa); #c1(RTN2) c2(NP_005610) c3(reticulon-2 isoform A) c4(157911, 157912) c5(157913, 157914, 157915, 157916, 157917, 157918, 157919) c6( ) c7(bkf, wc, U, Ni, cAA, awi); #c1(RTN2) c2(NP_996783) c3(reticulon-2 isoform B) c4(264527) c5(264528, 264529, 264530, 264531, 264532, 264533, 264534) c6( ) c7(bkf, wc, U, Ni, cAA, awi); #c1(RTN2) c2(NP_996784) c3(reticulon-2 isoform C) c4( ) c5(264535, 264536, 264537, 264538, 264539) c6( ) c7(bkf, wc, U, Ni, cAA, awi); #c1(RTN3) c2(NP_001252518) c3(reticulon-3 isoform e) c4(94059) c5(94060, 94061, 94062, 94063, 94064, 94065, 94066, 94067, 94068, 94069, 94070, 94071) c6( ) c7(iH, ds, Rr, oW, dV, gl, oi, eh, af); #c1(RTN3) c2(NP_001252520) c3(reticulon-3 isoform g) c4(94072) c5(94073, 94074, 94075, 94076, 94077, 94078, 94079, 94080, 94081, 94082, 94083, 94084) c6( ) c7(iH, ds, Rr, oW, dV, gl, oi, eh, af); #c1(RTN3) c2(NP_006045) c3(reticulon-3 isoform a) c4(161434, 161435) c5(161436, 161437, 161438, 161439, 161440, 161441, 161442, 161443, 161444, 161445, 161446, 161447) c6( ) c7(iH, ds, Rr, oW, dV, gl, oi, eh, af); #c1(RTN3) c2(NP_958831) c3(reticulon-3 isoform b) c4(262220) c5( ) c6( ) c7(iH, ds, Rr, oW, dV, gl, oi, eh, af); #c1(RTN3) c2(NP_958832) c3(reticulon-3 isoform c) c4(262221) c5( ) c6( ) c7(iH, ds, Rr, oW, dV, gl, oi, eh, af); #c1(RTN3) c2(NP_958833) c3(reticulon-3 isoform d) c4(262222) c5(262223, 262224, 262225, 262226, 262227, 262228) c6( ) c7(iH, ds, Rr, oW, dV, gl, oi, eh, af); #c1(RTN4) c2(NP_008939) c3(reticulon-4 isoform C) c4(169690) c5(169691, 169692, 169693, 169694, 169695, 169696) c6( ) c7(T, uG, jh, cz, il, Fc, V, q, eA, ft, ds, tT, anV, aT, bK, af, aa, je, bf, aW, ff, en, iM, c, mx, B); #c1(RTN4) c2(NP_065393) c3(reticulon-4 isoform A) c4(204701) c5(204702, 204703, 204704, 204705, 204706, 204707, 204708, 204709, 204710, 204711, 204712) c6( ) c7(T, uG, jh, cz, il, Fc, V, q, eA, ft, ds, tT, anV, aT, bK, af, aa, je, bf, aW, ff, en, iM, c, mx, B); #c1(RTN4) c2(NP_722550) c3(reticulon-4 isoform B) c4(246836, 246837) c5(246838, 246839, 246840, 246841, 246842, 246843, 246844, 246845, 246846, 246847, 246848, 246849) c6( ) c7(T, uG, jh, cz, il, Fc, V, q, eA, ft, ds, tT, anV, aT, bK, af, aa, je, bf, aW, ff, en, iM, c, mx, B); #c1(RTN4) c2(NP_997403) c3(reticulon-4 isoform D) c4(265529) c5(265530, 265531, 265532, 265533, 265534, 265535) c6( ) c7(T, uG, jh, cz, il, Fc, V, q, eA, ft, ds, tT, anV, aT, bK, af, aa, je, bf, aW, ff, en, iM, c, mx, B); #c1(RTN4) c2(XP_011531294) c3(reticulon-4 isoform X1) c4(327265, 327266, 327267) c5(327268, 327269, 327270, 327271, 327272, 327273, 327274, 327275, 327276, 327277, 327278, 327279, 327280, 327281, 327282, 327283, 327284, 327285, 327286, 327287, 327288, 327289, 327290, 327291, 327292) c6( ) c7(T, uG, jh, cz, il, Fc, V, q, eA, ft, ds, tT, anV, aT, bK, af, aa, je, bf, aW, ff, en, iM, c, mx, B); #c1(RTN4IP1) c2(NP_116119) c3(reticulon-4-interacting protein 1, mitochondrial precursor) c4(225407, 225408) c5(225409, 225410, 225411, 225412, 225413, 225414, 225415, 225416, 225417, 225418, 225419, 225420) c6( ) c7(T, hd, nM, nB); #c1(RTN4R) c2(NP_075380) c3(reticulon-4 receptor precursor) c4(213811) c5(213812, 213813, 213814, 213815, 213816, 213817, 213818, 213819) c6( ) c7(DA, cZ, Gt, T, af, agd, Y, RX, agb, gl, cm, eA, byQ, dd, he, V, q, cY); #c1(RTP3) c2(NP_113628) c3(receptor-transporting protein 3) c4(221810) c5(221811, 221812, 221813, 221814, 221815, 221816) c6( ) c7(aq, B, afl); #c1(RTP4) c2(NP_071430) c3(receptor-transporting protein 4) c4(211615) c5(211616, 211617, 211618, 211619, 211620, 211621) c6( ) c7(ds); #c1(RTTN) c2(NP_775901) c3(rotatin) c4(250626, 250627, 250628) c5(250629, 250630, 250631, 250632, 250633, 250634, 250635, 250636, 250637, 250638, 250639, 250640, 250641, 250642, 250643, 250644, 250645, 250646) c6( ) c7(cAB); #c1(RUFY1) c2(NP_001035542) c3(RUN and FYVE domain-containing protein 1 isoform b) c4(32901, 32902) c5( ) c6( ) c7(c); #c1(RUFY1) c2(NP_079434) c3(RUN and FYVE domain-containing protein 1 isoform a) c4(218120) c5(218121, 218122, 218123, 218124, 218125, 218126, 218127, 218128, 218129, 218130, 218131, 218132, 218133, 218134, 218135, 218136) c6( ) c7(c); #c1(RUFY1) c2(XP_006714985) c3(RUN and FYVE domain-containing protein 1 isoform X4) c4( ) c5(288378, 288379, 288380, 288381, 288382, 288383, 288384, 288385) c6( ) c7(c); #c1(RUFY1) c2(XP_011532960) c3(RUN and FYVE domain-containing protein 1 isoform X2) c4(329121) c5(329122, 329123, 329124, 329125, 329126) c6( ) c7(c); #c1(RUFY3) c2(NP_001032519) c3(protein RUFY3 isoform 1) c4(31351) c5(31352, 31353, 31354, 31355, 31356, 31357) c6( ) c7(Pk, e, lQ, an); #c1(RUFY3) c2(NP_001124181) c3(protein RUFY3 isoform 3) c4( ) c5(48435, 48436, 48437, 48438, 48439, 48440, 48441, 48442, 48443, 48444, 48445, 48446) c6( ) c7(Pk, e, lQ, an); #c1(RUFY3) c2(NP_001278923) c3(protein RUFY3 isoform 5) c4(111009) c5(111010, 111011, 111012, 111013, 111014, 111015) c6( ) c7(Pk, e, lQ, an); #c1(RUFY3) c2(NP_055776) c3(protein RUFY3 isoform 2) c4(183424) c5(183425, 183426, 183427, 183428, 183429, 183430, 183431) c6( ) c7(Pk, e, lQ, an); #c1(RUFY3) c2(XP_011530054) c3(protein RUFY3 isoform X4) c4(325880, 325881) c5( ) c6( ) c7(Pk, e, lQ, an); #c1(RUNDC3B) c2(NP_001127877) c3(RUN domain-containing protein 3B isoform b) c4(49418) c5(49419, 49420, 49421, 49422, 49423, 49424) c6( ) c7(aW, aT); #c1(RUNDC3B) c2(NP_001127878) c3(RUN domain-containing protein 3B isoform c) c4(49425) c5(49426, 49427, 49428, 49429, 49430, 49431) c6( ) c7(aW, aT); #c1(RUNDC3B) c2(NP_612147) c3(RUN domain-containing protein 38 isoform a) c4(236064) c5(236065, 236066, 236067, 236068, 236069, 236070) c6( ) c7(aW, aT); #c1(RUNDC3B) c2(XP_011514128) c3(RUN domain-containing protein 3B isoform X1) c4( ) c5(306736) c6( ) c7(aW, aT); #c1(RUNX1) c2(NP_001001890) c3(runt-related transcription factor 1 isoform AML1b) c4(19093) c5(19094, 19095, 19096, 19097, 19098, 19099, 19100, 19101, 19102, 19103, 19104) c6( ) c7(bM, T, os, aC, pf, alT, qx, al, fd, ci, x, oB, K, hH, aa, li, aW, ace, dg, as, Fu, bw, ou, iT, lq, dW, k, m, xu, zX, ht, n, oY, nA, pM, br, b, Dr, Bj, hn, DU, pi, aBl, Gc, wC, A, pm, cM, yR, Ye, bnN, hk, kz, hL, hR, pg, fL, bD, it, bL, fZ, aA, afK, j, Wa, q, er, eA, anj, oT, yg, aT, dz, jj, ae, lE, iU, iZ, Y, r, gm, en, hf, cl, t, f, bQ, Ci, ck, ac, ll, oX, bt, cAC, jh, bs, oZ, pz, hF, ad, dP, iC, fX, oW, iY, pq, il, et, ff, cr, eZ, gE, aF, iV, age, Ew, ll, i, y, kS); #c1(RUNX1) c2(NP_001116079) c3(runt-related transcription factor 1 isoform AML1a) c4(43397) c5(43398, 43399, 43400, 43401, 43402, 43403) c6( ) c7(bM, T, os, aC, pf, alT, qx, al, fd, ci, x, oB, K, hH, aa, li, aW, ace, dg, as, Fu, bw, ou, iT, lq, dW, k, m, xu, zX, ht, n, oY, nA, pM, br, b, Dr, Bj, hn, DU, pi, aBl, Gc, wC, A, pm, cM, yR, Ye, bnN, hk, kz, hL, hR, pg, fL, bD, it, bL, fZ, aA, afK, j, Wa, q, er, eA, anj, oT, yg, aT, dz, jj, ae, lE, iU, iZ, Y, r, gm, en, hf, cl, t, f, bl, Ci, ck, ac, ll, oX, bt, cAC, jh, bs, oZ, pz, hF, ad, dP, iC, fX, oW, iY, pq, il, et, ff, cr, eZ, gE, aF, iV, age, Ew, ll, i, y, kS); #c1 (RUNX1) c2(NP_001745) c3(runt-related transcription factor 1 isoform AML1c) c4(120692, 120693, 120694, 120695) c5(120696, 120697, 120698, 120699, 120700, 120701, 120702, 120703, 120704, 120705, 120706, 120707, 120708, 120709, 120710, 120711, 120712) c6( ) c7(bM, T, os, aC, pf, alT, qx, al, fd, ci, x, oB, K, hH, aa, li, aW, ace, dg, as, Fu, bw, ou, iT, lq, dW, k, m, xu, zX, ht, n, oY, nA, pM, br, b, Dr, Bj, hn, DU, pi, aBl, Gc, wC, A, pm, cM, yR, Ye, bnN, hk, kz, hL, hR, pg, fL, bD, it, bL, fZ, aA, afK, j, Wa, q, er, eA, anj, oT, yg, aT, dz, jj, ae, lE, iU, iZ, Y, r, gm, en, hf, cl, t, f, bQ, Ci, ck, ac, ll, oX, bt, cAC, jh, bs, oZ, pz, hF, ad, dP, iC, fX, oW, iY, pq, il, et, ff, cr, eZ, gE, aF, iV, age, Ew, ll, i, y, kS); #c1(RUNX1) c2(XP_005261125) c3(runt-related transcription factor 1 isoform X2) c4(277242) c5(277243, 277244, 277245, 277246, 277247, 277248) c6( ) c7(bM, T, os, aC, pf, alT, qx, al, fd, ci, x, oB, K, hH, aa, li, aW, ace, dg, as, Fu, bw, ou, iT, lq, dW, k, m, xu, zX, ht, n, oY, nA, pM, br, b, Dr, Bj, hn, DU, pi, aBl, Gc, wC, A, pm, cM, yR, Ye, bnN, hk, kz, hL, hR, pg, fL, bD, it, bL, fZ, aA, afK, j, Wa, q, er, eA, anj, oT, yg, aT, dz, jj, ae, lE, iU, iZ, Y, r, gm, en, hf, cl, t, f, bQ, Ci, ck, ac, ll, oX, bt, cAC, jh, bs, oZ, pz, hF, ad, dP, iC, fX, oW, iY, pq, il, et, ff, cr, eZ, gE, aF, iV, age, Ew, ll, i, y, kS); #c1(RUNX1) c2(XP_005261126) c3(runt-related transcription factor 1 isoform X4) c4(277249) c5( ) c6( ) c7(bM, T, os, aC, pf, alT, qx, al, fd, ci, x, OB, K, hH, aa, li, aW, ace, dg, as, Fu, bw, ou, iT, lq, dW, k, m, xu, zX, ht, n, oY, nA, pM, br, b, Dr, Bj, hn, DU, pi, aBl, Gc, wC, A, pm, cM, yR, Ye, bnN, hk, kz, hL, hR, pg, fL, bD, it, bL, fZ, aA, afK, j, Wa, q, er, eA, anj, oT, yg, aT, dz, jj, ae, lE, iU, iZ, Y, r, gm, en, hf, cl, t, f, bQ, Ci, ck, ac, ll, oX, bt, cAC, jh, bs, oZ, pz, hF, ad, dP, iC, fX, oW, iY, pq, il, et, ff, cr, eZ, gE, af, iV, age, Ew, ll, i, y, kS); #c1(RUNX1) c2(XP_011528071) c3(runt-related transcription factor 1 isoform X6) c4( ) c5(323147) c6( ) c7(bM, T, os, aC, pf, alT, qx, al, fd, ci, x, oB, K, hH, aa, li, aW, ace, dg, as, Fu, bw, ou, iT, lq, dW, k, m, xu, zX, ht, n, oY, nA, pM, br, b, Dr, Bj, hn, DU, pi, aBl, Gc, wC, A, pm, cM, yR, Ye, bnN, hk, kz, hL, hR, pg, fL, bD, it, bL, fZ, aA, afK, j, Wa, q, er, eA, anj, oT, yg, aT, dz, jj, ae, lE, iU, iZ, Y, r, gm, en, hf, cl, t, f, bl, Ci, ck, ac, ll, oX, bt, cAC, jh, bs, oZ, pz, hF, ad, dP, iC, fX, oW, iY, pq, il, et, ff, cr, eZ, gE, aF, iV, age, Ew, ll, i, y, kS); #c1(RUNX1) c2(XP_011528072) c3(runt-related transcription factor 1 isoform X7) c4( ) c5(323148, 323149, 323150, 323151, 323152, 323153, 323154, 323155, 323156, 323157, 323158, 323159) c6( ) c7(bM, T, os, aC, pf, alT, qx, al, fd, ci, x, oB, K, hH, aa, li, aW, ace, dg, as, Fu, bw, ou, iT, lq, dW, k, m, xu, zX, ht, n, oY, nA, pM, br, b, Dr, Bj, hn, DU, pi, aBl, Gc, wC, A, pm, cM, yR, Ye, bnN, hk, kz, hL, hR, pg, fL, bD, it, bL, fZ, aA, afK, j, Wa, q, er, eA, anj, oT, yg, aT, dz, jj, ae, lE, iU, iZ, Y, r, gm, en, hf, cl, t, f, bQ, Ci, ck, ac, ll, oX, bt, cAC, jh, bs, oZ, pz, hF, ad, dP, iC, fX, oW, iY, pq, il, et, ff, cr, eZ, gE, aF, iV, age, Ew, ll, i, y, kS); #c1(RUNX1T1) c2(NP_001185560) c3(protein CBFAZT1 isoform B) c4(75854, 75855, 75856, 75857, 75858, 75859, 75860, 75861, 75862, 75863, 75864, 75865, 75866, 75867, 75868, 75869) c5(75870, 75871, 75872, 75873, 75874, 75875, 75876, 75877, 75878, 75879, 75880, 75881, 75882, 75883, 75884, 75885, 75886, 75887, 75888, 75889, 75890, 75891, 75892, 75893, 75894, 75895, 75896, 75897, 75898, 75899, 75900, 75901, 75902, 75903, 75904, 75905, 75906, 75907, 75908, 75909, 75910, 75911, 75912, 75913, 75914, 75915, 75916, 75917, 75918, 75919, 75920, 75921, 75922, 75923, 75924, 75925, 75926, 75927, 75928, 75929, 75930, 75931, 75932, 75933, 75934, 75935, 75936, 75937, 75938, 75939, 75940, 75941, 75942, 75943, 75944, 75945, 75946, 75947, 75948, 75949, 75950, 75951, 75952, 75953) c6( ) c7(oO, T, fM, aw, bs, fd, n, hF, br, er, x, b, fx, ft, Do, sO, Bj, aT, h, dz, hn, iY, jj, pi, aBl, MG, iU, et, aW, Y, lo, PG, pm, f, bw, yR, ck, ou, ac, pc, iT, cj, k, rH, i, y, bt, kS, L); #c1(RUNX1T1) c2(NP_001185562) c3(protein CBFAZT1 isoform D) c4( ) c5(75954) c6( ) c7(oO, T, fM, aw, bs, fd, n, hF, br, er, x, b, fX, ft, Do, sO, Bj, aT, h, dz, hn, iY, jj, pi, aBl, MG, iU, et, aW, Y, lo, PG, pm, f, bw, yR, ck, ou, ac, pc, iT, cj, k, rH, i, y, bt, kS, L); #c1(RUNX1T1) c2(NP_001185563) c3(protein CBFAZT1 isoform E) c4(75955) c5(75956, 75957, 75958, 75959, 75960, 75961, 75962, 75963, 75964) c6( ) c7(oO, T, fM, aw, bs, fd, n, hF, br, er, x, b, fx, ft, Do, sO, Bj, aT, h, dz, hn, iY, jj, pi, aBl, MG, il, et, aW, Y, lo, PG, pm, f, bw, yR, ck, ou, ac, pc, iT, cj, k, rH, i, y, bt, kS, L); #c1(RUNX1T1) c2(NP_001185608) c3(protein CBFA2T1 isoform F) c4( ) c5(75979, 75980, 75981, 75982, 75983, 75984, 75985, 75986, 75987, 75988, 75989) c6( ) c7(oO, T, fM, aw, bs, fd, n, hF, br, er, x, b, fX, ft, Do, sO, Bj, aT, h, dz, hn, iY, jj, pi, aBl, MG, iU, et, aW, Y, lo, PG, pm, f, bw, yR, ck, ou, ac, pc, iT, cj, k, rH, i, y, bt, kS, L); #c1(RUNX1T1) c2(NP_004340) c3(protein CBFAZT1 isoform A) c4(145480, 145481, 145482, 145483, 145484, 145485) c5(145486, 145487, 145488, 145489, 145490, 145491, 145492, 145493, 145494, 145495, 145496, 145497, 145498, 145499, 145500, 145501, 145502, 145503, 145504, 145505, 145506, 145507, 145508, 145509, 145510, 145511, 145512, 145513, 145514, 145515, 145516, 145517, 145518) c6( ) c7(oO, T, fM, aw, bs, fd, n, hF, br, er, x, b, fx, ft, Do, sO, Bj, aT, h, dz, hn, iY, jj, pi, aBl, MG, iU, et, aW, Y, lo, PG, pm, f, bw, yR, ck, ou, ac, pc, iT, cj, k, rH, i, y, bt, kS, L); #c1(RUNX1T1) c2(NP_783553) c3(protein CBFAZT1 isoform C) c4( ) c5(251678, 251679, 251680, 251681, 251682, 251683, 251684, 251685, 251686, 251687, 251688, 251689, 251690, 251691, 251692, 251693, 251694, 251695, 251696, 251697) c6( ) c7(oO, T, fM, aw, bs, fd, n, hF, br, er, x, b, fx, ft, Do, sO, Bj, aT, h, dz, hn, iY, jj, pi, aBl, MG, il, et, aW, Y, lo, PG, pm, f, bw, yR, ck, ou, ac, pc, iT, cj, k, rH, i, y, bt, KS, L); #c1(RUNX2) c2(NP_001015051) c3(runt-related transcription factor 2 isoform b) c4(26157) c5(26158, 26159, 26160, 26161, 26162, 26163) c6( ) c7(gh, T, fd, cAG, x, Na, aE, arA, K, pr, aW, lW, Az, PQ, g, eF, bw, zV, cAE, eM, nB, iA, lq, asR, m, zt, n, Kw, ai, adf, b, zw, ask, Wr, aq, kp, wU, bP, hR, art, B, vQ, bL, AS, bDS, aA, wo, cAF, q, eA, cAD, yg, aT, Nc, jj, ae, af, bA, Y, r, akB, bQ, t, ck, Qi, Ta, acW, nM, cgp, aru, bt, kh, jh, dG, hF, ad, hd, Db, oj, iL, d, eH, dr, es, yE, bE, Fk, i, iu, Tc); #c1(RUNX2) c2(NP_001019801) c3(runt-related transcription factor 2 isoform a) c4( ) c5(27886, 27887, 27888, 27889, 27890, 27891, 27892, 27893, 27894, 27895, 27896, 27897, 27898, 27899, 27900) c6( ) c7(gh, T, fd, cAG, x, Na, aE, arA, K, pr, aW, lW, Az, PQ, g, eF, bw, zV, cAE, eM, nB, iA, lq, asR, m, zt, n, Kw, ai, adf, b, zw, ask, Wr, aq, kp, wU, bP, hR, art, B, vQ, bL, AS, bDS, aA, wo, cAF, q, eA, cAD, yg, aT, Nc, jj, ae, af, bA, Y, r, akB, bQ, t, ck, Qi, Ta, acW, nM, cgp, aru, bt, kh, jh, dG, hF, ad, hd, Db, oj, iL, d, eH, dr, es, yE, bE, Fk, i, iu, Tc); #c1(RUNX2) c2(NP_001265407) c3(runt-related transcription factor 2 isoform d) c4(98533) c5(98534, 98535, 98536, 98537, 98538, 98539) c6( ) c7(gh, T, fd, cAG, x, Na, aE, arA, K, pr, aW, lW, Az, PQ, g, eF, bw, zV, cAE, eM, nB, iA, lq, asR, m, zt, n, Kw, ai, adf, b, zw, ask, Wr, aq, kp, wU, bP, hR, art, B, vQ, bL, AS, bDS, aA, wo, cAF, q, eA, cAD, yg, aT, Nc, jj, ae, af, bA, Y, r, akB, bQ, t, ck, Qi, Ta, acW, nM, cgp, aru, bt, kh, jh, dG, hF, ad, hd, Db, oj, iL, d, eH, dr, es, yE, bE, Fk, i, iu, Tc); #c1(RUNX2) c2(XP_011513263) c3(runt-related transcription factor 2 isoform X2) c4(305524, 305525, 305526) c5(305527, 305528, 305529, 305530, 305531, 305532, 305533, 305534) c6( ) c7(gh, T, fd, cAG, x, Na, aE, arA, K, pr, aW, lW, Az, PQ, g, eF, bw, zV, cAE, eM, nB, iA, lq, asR, m, zt, n, Kw, ai, adf, b, zw, ask, Wr, aq, kp, wU, bP, hR, art, B, vQ, bL, AS, bDS, aA, wo, cAF, q, eA, cAD, yg, aT, Nc, jj, ae, af, bA, Y, r, akB, bQ, t, ck, Qi, Ta, acW, nM, cgp, aru, bt, kh, jh, dG, hF, ad, hd, Db, oj, iL, d, eH, dr, es, yE, bE, Fk, i, iu, Tc); #c1(RUNX2) c2(XP_011513264) c3(runt-related transcription factor 2 isoform X3) c4(305535) c5(305536, 305537, 305538, 305539, 305540, 305541) c6( ) c7(gh, T, fd, cAG, x, Na, aE, arA, K, pr, aW, lW, Az, PQ, g, eF, bw, zV, cAE, eM, nB, iA, lq, asR, m, zt, n, Kw, ai, adf, b, zw, ask, Wr, aq, kp, wU, bP, hR, art, B, vQ, bL, AS, bDS, aA, wo, cAF, q, eA, cAD, yg, aT, Nc, jj, ae, af, bA, Y, r, akB, bQ, t, ck, Qi, Ta, acW, nM, cgp, aru, bt, kh, jh, dG, hF, ad, hd, Db, oj, iL, d, eH, dr, es, yE, bE, Fk, i, iu, Tc); #c1(RUNX2) c2(XP_011513268) c3 (runt-related transcription factor 2 isoform X8) c4( ) c5(305542, 305543, 305544) c6( ) c7(gh, T, fd, cAG, x, Na, aE, arA, K, pr, aW, lW, Az, PQ, g, eF, bw, zV, cAE, eM, nB, iA, lq, asR, m, zt, n, Kw, ai, adf, b, zw, ask, Wr, aq, kp, wU, bP, hR, art, B, vQ, bL, AS, bDS, aA, wo, cAF, q, eA, cAD, yg, aT, Nc, jj, ae, af, bA, Y, r, akB, bQ, t, ck, Qi, Ta, acW, nM, cgp, aru, bt, kh, jh, dG, hF, ad, hd, Db, oj, iL, d, eH, dr, es, yE, bE, Fk, i, iu, Tc); #c1(RUNX3) c2(XP_011540653) c3(runt-related transcription factor 3 isoform X2) c4(339206) c5(339207, 339208, 339209, 339210, 339211, 339212) c6( ) c7(T, aC, fR, pf, bB, ci, aE, K, hu, eL, cp, Qe, aW, qS, az, LZ, nY, kt, aB, g, cm, arZ, eM, Jr, m, cAH, cZ, iB, ht, n, fr, qN, dL, dd, dx, mR, bVg, aCx, ge, fe, aq, mo, hL, it, B, aA, il, AO, pd, q, cF, afQ, BJ, qK, aT, ae, amw, kl, s, vT, oV, Y, r, en, cl, t, sS, Dy, gX, zu, KY, rA, bt, vi, aD, jh, Gn, jE, hF, ad, afb, wD, av, ji, d, qt, ff, cr, aF, hp, iV, bz, bF, bE, X, iu); #c1(RUVBL1) c2(NP_003698) c3(ruvB-like 1) c4(140242, 140243) c5(140244, 140245, 140246, 140247, 140248, 140249) c6( ) c7(T, aE, bE, aW, if, aT, bB, By, m, qN, t, X, ad); #c1(RUVBL1) c2(XP_011511551) c3(ruvB-like 1 isoform X3) c4( ) c5(302257, 302258, 302259, 302260, 302261, 302262) c6( ) c7(T, aE, bE, aW, if, aT, bB, By, m, qN, t, X, ad); #c1(RUVBL2) c2(NP_006657) c3(ruvB-like 2) c4(166953) c5(166954, 166955, 166956, 166957, 166958, 166959, 166960, 166961, 166962, 166963, 166964) c6( ) c7(ck, T, ac, bE, oV, M, bB, B, eu); #c1(RUVBL2) c2(XP_005258484) c3(ruvB-like 2 isoform X1) c4(275681) c5( ) c6( ) c7(ck, T, ac, bE, oV, M, bB, B, eu); #c1(RUVBL2) c2(XP_011524632) c3(ruvB-like 2 isoform X2) c4( ) c5(319172, 319173, 319174, 319175, 319176) c6( ) c7(ck, T, ac, bE, oV, M, bB, B, eu); #c1(RXFP1) c2(NP_001240658) c3(relaxin receptor 1 isoform 4) c4( ) c5(88679, 88680, 88681, 88682, 88683, 88684, 88685, 88686, 88687, 88688, 88689) c6( ) c7(dP, qJ, gk, T, hd, nv, op, vA, al, cl, ae, ad); #c1(RXFP1) c2(NP_001240661) c3(relaxin receptor 1 isoform 6) c4(88690) c5(88691, 88692, 88693, 88694, 88695, 88696) c6( ) c7(dP, qJ, gk, T, hd, nv, op, vA, al, cl, ae, ad); #c1(RXFP1) c2(NP_067647) c3(relaxin receptor 1 isoform 1) c4(209216, 209217) c5(209218, 209219, 209220, 209221, 209222, 209223, 209224) c6( ) c7(dP, qJ, gk, T, hd, nv, op, vA, al, cl, ae, ad); #c1(RXFP1) c2(XP_011530478) c3(relaxin receptor 1 isoform X3) c4(326370, 326371) c5(326372, 326373, 326374, 326375, 326376, 326377) c6( ) c7(dP, qJ, gk, T, hd, nv, op, vA, al, cl, ae, ad); #c1(RXFP2) c2(NP_001159530) c3(relaxin receptor 2 isoform 2) c4(64258) c5(64259, 64260, 64261, 64262, 64263, 64264) c6( ) c7(gk, yi, nv, Lf, vA, al, bu, ae, op, bA, UF); #c1(RXFP2) c2(NP_570718) c3(relaxin receptor 2 isoform 1) c4(234224) c5(234225, 234226, 234227, 234228, 234229, 234230) c6( ) c7(gk, yi, nv, Lf, vA, al, bu, ae, op, bA, UF); #c1(RXFP3) c2(NP_057652) c3(relaxin-3 receptor 1) c4(192063) c5(192064, 192065, 192066, 192067, 192068, 192069) c6( ) c7(v, K, cl, hq); #c1(RXRA) c2(NP_001278849) c3(retinoic acid receptor RXR-alpha isoform b) c4( ) c5(110720) c6( ) c7(bL, vq, T, AS, jp, My, bB, n, hy, v, j, ci, C, PJ, Fb, zP, fr, q, cd, ad, hX, Mg, av, l, hd, aE, u, lt, aT, ab, dL, UN, jj, hB, nJ, ae, af, eN, B, BN, s, aW, ff, cr, r, iy, aq, eZ, CQ, en, cM, bz, op, ck, es, eM, nB, bE, vA, X, wk, at, MJ, lC, bt, ne); #c1(RXRA) c2(NP_001278850) c3(retinoic acid receptor RXR-alpha isoform c) c4( ) c5(110721, 110722, 110723, 110724, 110725) c6( ) c7(bL, vq, T, AS, jp, My, bB, n, hy, v, j, ci, C, PJ, Fb, zP, fr, q, cd, ad, hX, Mg, av, l, hd, aE, u, lt, aT, ab, dL, UN, jj, hB, nJ, ae, af, eN, B, BN, s, aW, ff, cr, r, iy, aq, eZ, CQ, en, cM, bz, op, ck, es, eM, nB, bE, vA, X, wk, at, MJ, lC, bt, ne); #c1(RXRA) c2(NP_002948) c3(retinoic acid receptor RXR-alpha isoform a) c4(133837, 133838) c5(133839, 133840, 133841, 133842, 133843, 133844, 133845) c6( ) c7(bL, vq, T, AS, jp, My, bB, n, hy, v, j, ci, C, PJ, Fb, zP, fr, q, cd, ad, hX, Mg, av, l, hd, aE, u, lt, aT, ab, dL, UN, jj, hB, nJ, ae, af, eN, B, BN, s, aW, ff, cr, r, iy, aq, eZ, CQ, en, cM, bz, op, ck, es, eM, nB, bE, vA, X, wk, at, MJ, lC, bt, ne); #c1(RXRB) c2(NP_001257330) c3(retinoic acid receptor RXR-beta isoform 1) c4(95187) c5( ) c6( ) c7(AS, iW, aA, ad, av, l, hd, u, lt, aT, dL, hB, af, A, aW, cr, eZ, cm, ve, bIS, at, hv); #c1(RXRB) c2(NP_001278918) c3(retinoic acid receptor RXR-beta isoform 3) c4(110971) c5(110972, 110973, 110974, 110975, 110976, 110977, 110978, 110979, 110980, 110981, 110982, 110983, 110984, 110985, 110986, 110987, 110988, 110989, 110990) c6( ) c7(AS, iW, aA, ad, av, l, hd, u, lt, aT, dL, hB, af, A, aW, cr, eZ, cm, ve, bIS, at, hv); #c1(RXRB) c2(NP_06881) c3(retinoic acid receptor RXR-beta isoform 2) c4(210700, 210701, 210702) c5(210703, 210704, 210705, 210706, 210707, 210708, 210709, 210710, 210711, 210712, 210713, 210714, 210715, 210716, 210717, 210718, 210719, 210720, 210721, 210722, 210723, 210724, 210725, 210726, 210727, 210728, 210729, 210730, 210731, 210732, 210733, 210734, 210735, 210736, 210737, 210738, 210739, 210740, 210741, 210742, 210743, 210744, 210745, 210746, 210747, 210748, 210749, 210750, 210751, 210752, 210753, 210754, 210755, 210756, 210757, 210758, 210759, 210760, 210761, 210762, 210763, 210764, 210765, 210766, 210767, 210768, 210769, 210770) c6( ) c7(AS, iW, aA, ad, av, l, hd, u, lt, aT, dL, hB, af, A, aW, cr, eZ, cm, ve, bIS, at, hv); #c1(RXRB) c2(XP_005249335) c3(retinoic acid receptor RXR-beta isoform X1) c4(269177) c5(269178, 269179, 269180, 269181, 269182, 269183, 269184, 269185, 269186, 269187, 269188, 269189, 269190, 269191, 269192, 269193, 269194, 269195, 269196, 269197, 269198, 269199, 269200, 269201) c6( ) c7(AS, iW, aA, ad, av, l, hd, u, lt, aT, dL, hB, af, A, aW, cr, eZ, cm, ve, bIS, at, hv); #c1(RXRB) c2(XP_005249336) c3(retinoic acid receptor RXR-beta isoform X3) c4(269202) c5(269203, 269204, 269205, 269206, 269207, 269208, 269209, 269210, 269211, 269212, 269213, 269214, 269215, 269216, 269217, 269218, 269219, 269220, 269221, 269222, 269223, 269224, 269225, 269226, 269227, 269228, 269229, 269230, 269231, 269232, 269233, 269234, 269235, 269236, 269237, 269238) c6( ) c7(AS, iW, aA, ad, av, l, hd, u, lt, aT, dL, hB, af, A, aW, cr, eZ, cm, ve, bIS, at, hv); #c1(RXRG) c2(NP_008848) c3(retinoic acid receptor RXR-gamma isoform a) c4(169091) c5(169092, 169093, 169094, 169095, 169096, 169097, 169098) c6( ) c7(bL, bA, T, il, nB, bE, Fi, cz, bB, aj, v, j, fr, af, J, na); #c1(RYBP) c2(NP_036366) c3(RING1 and YY1-binding protein) c4(173267) c5(173268, 173269, 173270, 173271, 173272, 173273, 173274) c6( ) c7(T, iM, je, bK, hH, fq); #c1(RYK) c2(NP_001005861) c3(tyrosine-protein kinase RYK isoform 1 precursor) c4(22129, 22130) c5(22131, 22132, 22133, 22134, 22135, 22136, 22137, 22138, 22139, 22140, 22141, 22142) c6( ) c7(Na, bxy, Az, Nc, m, t); #c1(RYK) c2(NP_002949) c3(tyrosine-protein kinase RYK isoform 2 precursor) c4(133846) c5(133847, 133848, 133849, 133850, 133851, 133852) c6( ) c7(Na, bxy, Az, Nc, m, t); #c1(RYR1) c2(NP_000531) c3(ryanodine receptor 1 isoform 1) c4(12022) c5(12023, 12024, 12025, 12026, 12027, 12028, 12029, 12030, 12031, 12032, 12033, 12034, 12035, 12036, 12037, 12038, 12039, 12040) c6( ) c7(lD, cAl, T, fR, e, Am, bMY, cz, jh, fr, UX, cd, ad, xC, NW, no, ayH, oj, Ak, h, aWs, K, bDK, Aa, ae, aTm, ly, Al, xi, vj, xz, lW, Ad, cAL, cmG, akB, cAK, Bu, lx, aPd, dR, lJ, ac, ayl, cAJ, VU, jQ, y, jr); #c1(RYR1) c2(NP_001036188) c3(ryanodine receptor 1 isoform 2) c4(34033) c5(34034, 34035, 34036, 34037, 34038, 34039) c6( ) c7(lD, cAl, T, FR, e, Am, bMY, cz, jh, fr, UX, cd, ad, xC, NW, no, ayH, oj, Ak, h, aWs, K, bDK, Aa, ae, aTm, ly, Al, xi, vj, xz, lW, Ad, cAL, cmG, akB, cAK, Bu, lx, aPd, dR, lJ, ac, ayl, cAJ, VU, jQ, y, jr); #c1(RYR1) c2(XP_006723380) c3(ryanodine receptor 1 isoform X2) c4(295186) c5( ) c6( ) c7(lD, cAl, T, FR, e, Am, bMY, cz, jh, fr, UX, cd, ad, xC, NW, no, ayH, oj, Ak, h, aWs, K, bDK, Aa, ae, aTm, ly, Al, xi, vj, xz, lW, Ad, cAL, cmG, akB, cAK, Bu, lx, aPd, dR, lJ, ac, ayl, cAJ, VU, jQ, y, jr); #c1(RYR1) c2(XP_011525507) c3(ryanodine receptor 1 isoform X4) c4(319907) c5(319908) c6( ) c7(lD, cAl, T, fR, e, Am, bMY, cz, jh, fr, UX, cd, ad, xC, NW, no, ayH, oj, Ak, h, aWs, K, bDK, Aa, ae, aTm, ly, Al, xi, vj, xz, lW, Ad, cAL, cmG, akB, cAK, Bu, lx, aPd, dR, lJ, ac, ayl, cAJ, VU, jQ, y, jr); #c1(RYR2) c2(XP_006711865) c3(ryanodine receptor 2 isoform X1) c4( ) c5(286240, 286241, 286242, 286243, 286244, 286245, 286246, 286247, 286248, 286249) c6( ) c7(cAP, cAM, mr, wL, acl, oe, e, EQ, cV, arD, tc, bhL, cz, aj, j, atN, sl, cAN, V, cd, J, Mg, cc, oj, h, vc, hn, K, bDK, bq, cAQ, af, mu, VX, Y, vj, lW, akB, EZ, cdk, wK, bVf, mx, gZ, bUH, cAD, dy, bD, lC, gq, rf); #c1(RYR2) c2(XP_006711866) c3(ryanodine receptor 2 isoform X2) c4( ) c5(286250) c6( ) c7(cAP, cAM, mr, wL, acl, oe, e, EQ, cV, arD, tc, bhL, cz, aj, j, atN, sl, cAN, V, cd, J, Mg, cc, oj, h, vc, hn, K, bDK, bq, cAl, af, mu, VX, Y, vj, lW, akB, EZ, cdk, wK, bVf, mx, gZ, bUH, cAO, dy, bD, lC, gq, rf); #c1(RYR2) c2(XP_006711867) c3(ryanodine receptor 2 isoform X3) c4(286251) c5( ) c6( ) c7(cAP, cAM, mr, wL, acl, oe, e, EQ, cV, arD, tc, bhL, cz, aj, j, atN, sl, cAN, V, cd, J, Mg, cc, oj, h, vc, hn, K, bDK, bq, cAd, af, mu, VX, Y, vj, lW, akB, EZ, cdk, wK, bVf, mx, gZ, bUH, cAO, dy, bD, lC, gq, rf); #c1(RYR2) c2(XP_006711868) c3(ryanodine receptor 2 isoform X4) c4(286252) c5(286253, 286254, 286255, 286256, 286257, 286258, 286259) c6( ) c7(cAP, cAM, mr, wL, acl, oe, e, EQ, cV, arD, tc, bhL, cz, aj, j, atN, sl, cAN, V, cd, J, Mg, cc, oj, h, vc, hn, K, bDK, bq, cAQ, af, mu, VX, Y, vj, lW, akB, EZ, cdk, wK, bVf, mx, gZ, bUH, cAO, dy, bD, lC, gq, rf); #c1(RYR2) c2(XP_006711871) c3 (ryanodine receptor 2 isoform X7) c4(286260, 286261) c5(286262, 286263, 286264, 286265, 286266, 286267) c6( ) c7(cAP, cAM, mr, wL, acl, oe, e, EQ, cV, arD, tc, bhL, cz, aj, j, atN, sl, cAN, V, cd, J, Mg, cc, oj, h, vc, hn, K, bDK, bq, cAQ, af, mu, VX, Y, vj, lW, akB, EZ, cdk, wK, bVf, mx, gZ, bUH, cAO, dy, bD, lC, gq, rf); #c1(RYR2) c2(XP_006711872) c3(ryanodine receptor 2 isoform X8) c4( ) c5(286268, 286269) c6( ) c7(cAP, cAM, mr, wL, acl, oe, e, EQ, cV, arD, tc, bhL, cz, aj, j, atN, sl, cAN, V, cd, J, Mg, cc, oj, h, vc, hn, K, bDK, bq, cAQ, af, mu, VX, Y, vj, lW, akB, EZ, cdk, wK, bVf, mx, gZ, bUH, cAO, dy, bD, lC, gq, rf); #c1(RYR3) c2(NP_001027) c3 (ryanodine receptor 3 isoform 1) c4(30322) c5( ) c6( ) c7(dD, ac, dY, ea, vj, e, aw, cz, h, j, K, eq, iG, rE, cd, bN, qc); #c1(RYR3) c2(NP_001230925) c3(ryanodine receptor 3 isoform 2) c4(87177, 87178) c5(87179, 87180, 87181, 87182, 87183, 87184, 87185, 87186, 87187, 87188, 87189, 87190) c6( ) c7(dD, ac, dY, ea, vj, e, aw, cz, h, j, K, eq, iG, rE, cd, bN, qc); #c1(S100A1) c2(NP_006262) c3(protein S100-A1) c4(163365, 163366, 163367, 163368) c5(163369, 163370, 163371, 163372, 163373, 163374, 163375, 163376, 163377, 163378, 163379, 163380, 163381, 163382, 163383, 163384, 163385, 163386, 163387, 163388, 163389, 163390, 163391, 163392) c6( ) c7(bM, T, aNA, Tb, apr, ea, if, e, chc, qj, ci, ip, aE, aeO, SV, Ou, H, eL, cp, yo, aeR, LC, aW, Ls, ih, g, eE, eF, cm, eM, iA, ki, cAS, mx, U, Qw, PV, m, dD, Uc, WA, aw, n, aj, fr, ct, qN, cd, asS, ai, eW, Bl, cc, kH, h, dL, dx, Yl, Lg, kj, fe, aq, kp, anx, it, B, L, ka, jx, aA, CR, cz, hz, V, avD, q, J, eA, aaF, acj, qK, aT, avt, ae, qk, jf, iF, cAR, Y, lo, Xk, en, cl, t, f, acW, Dy, ac, iM, wR, CZ, auN, fD, aps, id, atY, rQ, aPJ, rl, bS, eq, ad, iC, Tg, Rq, Db, hD, hP, fb, d, je, afe, ff, eH, cr, rM, asV, aF, aiz, iV, Z, adj, es, Ew, VO, MJ, aS, ra, y, xg, iu, pC); #c1(S100A10) c2(NP_002957) c3(protein S100-A10) c4(133926) c5(133927, 133928, 133929, 133930, 133931, 133932, 133933, 133934, 133935, 133936) c6( ) c7(T, ha, bO, aA, n, bS, fr, ct, ci, q, ip, Db, aT, h, bK, K, he, jf, d, r, fe, aF, aFU, Z, eE, hq, hc, ki, Ei, U, hR, wR, ra, zu, bt, it); #c1(S100A11) c2(NP_005611) c3(protein S100-All) c4(157920) c5(157921, 157922, 157923, 157924, 157925, 157926) c6( ) c7(T, aA, ci, q, ad, Zp, av, aT, nm, By, dL, eL, ae, aW, en, g, eE, t, op, nM, vA, m, it, B); #c1(S100A12) c2(NP_005612) c3 (protein S100-A12) c4(157927, 157928, 157929) c5(157930, 157931, 157932, 157933, 157934, 157935, 157936, 157937, 157938, 157939, 157940, 157941) c6( ) c7(bM, cZ, dD, cAT, azf, tQ, Zl, ea, cz, cL, aj, j, uu, v, eq, fo, J, wD, iC, hd, py, nv, aqJ, aT, Aa, K, dd, H, avn, af, bA, azh, Nr, A, bf, aW, ih, eH, fe, hv, po, iV, eF, cm, bz, qa, Nq, kT, ok, y, cA, nB, eM, z, ki, dh, QF, Gg, fD, be, lM, lf, bt, vF, fl); #c1(S100A13) c2(XP_011508166) c3 (protein S100-A13 isoform X1) c4(298678) c5(298679, 298680, 298681, 298682, 298683, 298684, 298685, 298686, 298687, 298688, 298689, 298690, 298691, 298692, 298693, 298694, 298695, 298696, 298697, 298698, 298699, 298700) c6( ) c7(Dy, Y, ki, as, bz, eA); #c1(S100A14) c2(XP_005245419) c3(protein S100-A14 isoform X1) c4(266521) c5(266522, 266523, 266524, 266525, 266526, 266527, 266528, 266529, 266530, 266531, 266532, 266533, 266534, 266535, 266536, 266537, 266538, 266539) c6( ) c7(T, eM, aW, eE, ht, ff, cr, aT, it, ci, lQ, Xk, hu, en, bz); #c1(S100A16) c2(NP_525127) c3(protein S100-A16) c4( ) c5(231217, 231218, 231219, 231220, 231221, 231222, 231223, 231224, 231225, 231226, 231227, 231228, 231229, 231230) c6( ) c7(T, eA); #c1(S100A16) c2(XP_005244963) c3(protein S100-A16 isoform X2) c4(266167) c5(266168, 266169, 266170, 266171) c6( ) c7(T, eA); #c1(S100A2) c2(NP_005969) c3(protein S100-A2) c4(160862, 160863) c5(160864, 160865, 160866, 160867, 160868, 160869, 160870, 160871, 160872, 160873, 160874, 160875, 160876, 160877, 160878, 160879, 160880, 160881, 160882, 160883) c6( ) c7(T, aA, n, hy, ci, ad, iC, av, Bl, cF, aE, aT, ab, ae, jf, iE, aW, Y, ff, aF, en, mo, eE, g, ik, bz, iP, eM, afd, PV, it, iu); #c1(S100A3) c2(NP_002951) c3(protein S100-A3) c4(133853) c5(133854, 133855, 133856, 133857, 133858, 133859, 133860, 133861, 133862, 133863) c6( ) c7(eE, aF, aA); #c1(S100A4) c2(NP_002952) c3(protein S100-A4) c4(133864, 133865) c5(133866, 133867, 133868, 133869, 133870, 133871, 133872, 133873, 133874, 133875, 133876, 13387, 133878, 133879, 133880, 133881, 133882, 133883, 133884, 133885, 133886, 133887, 133888, 133889) c6( ) c7(T, aC, al, bB, zh, ci, ip, x, Vp, gJ, aE, rJ, K, hu, eL, yo, aW, hl, Qm, g, as, eF, eE, cm, eM, nB, vA, Hb, k, m, iu, sc, aiZ, Vo, zX, ht, qy, n, Bl, dL, SD, wC, lb, op, bP, dB, hk, ajd, hL, hR, it, B, fG, aA, q, eA, BJ, acj, Vr, aT, cy, Fx, ae, jf, s, Y, ev, r, en, hf, cl, LV, t, iK, ck, cT, nM, Dy, iM, bt, id, jh, bS, Fb, ad, hX, dP, iC, hd, ib, Db, yt, ab, d, lU, je, ff, eH, cr, aeV, aoW, aF, bz, LB, es, bE, aS, adg, lC, X, y); #c1(S100A5) c2(NP_002953) c3(protein S100-A5) c4( ) c5(133890, 133891, 133892, 133893, 133894, 133895, 133896, 133897) c6( ) c7(BE, ip); #c1(S100A5) c2(XP_011508162) c3(protein S100-A5 isoform X1) c4(298667, 298668) c5(298669, 298670, 298671, 298672, 298673, 298674, 298675) c6( ) c7(eE, ip); #c1(S100A6) c2(NP_055439) c3(protein S100-A6) c4(181140) c5(181141, 181142, 181143, 181144, 181145, 181146, 181147, 181148, 181149, 181150) c6( ) c7(bL, T, qn, aC, dA, nl, boK, eP, aA, cz, eb, Fb, ci, zP, q, ad, eA, hX, ip, iC, AW, Yn, gJ, acj, x, afQ, aT, By, K, Dz, bq, Fg, ae, af, jf, zQ, d, iU, aW, Y, ff, eH, aq, aF, Xk, en, cl, t, g, eE, eF, qa, cm, iR, ov, nM, c, nB, hL, k, m, bt, B); #c1(S100A7) c2(NP_002954) c3(protein S100-A7) c4(133898, 133899) c5(133900, 133901, 133902, 133903, 133904, 133905, 133906, 133907, 133908, 133909) c6( ) c7(T, yk, aee, aA, n, ad, aEQ, Bl, afr, aT, dL, K, lQ, ae, eL, aW, ff, azP, cr, dN, aF, Xk, afs, en, t, eE, ik, pb, sS, hk, JM, in); #c1(S100A7A) c2(NP_789793) c3(protein S100-A7A) c4(252319) c5(252320, 252321, 252322, 252323, 252324, 252325, 252326, 252327, 252328, 252329, 252330, 252331, 252332, 252333) c6( ) c7(T, kl, eM, ff, cr, aT, lQ, Xk, en, bz); #c1(S100A8) c2(NP_002955) c3(protein S100-A8) c4(133910) c5(133911, 133912, 133913, 133914, 133915, 133916, 133917, 133918) c6( ) c7(oO, bM, T, ea, if, e, cL, ci, bh, Tz, ip, aE, dV, K, H, hH, eL, bc, aiJ, Us, hm, UC, li, yo, zQ, Jl, aW, lh, az, HA, kt, pD, aB, g, eE, eF, cm, y, eM, iT, dc, U, cj, uJ, Uz, eg, cE, dD, aw, Wb, aj, qN, KY, ai, h, dL, hB, pl, A, lb, fe, kT, dB, hk, hL, at, Br, uz, eo, Pr, B, it, L, bL, pF, mr, aA, cR, fF, v, j, C, AO, V, q, o, J, mj, lg, qp, ld, aT, cy, jj, kU, ae, dl, le, kl, s, Y, kX, FO, la, r, akY, lo, en, iK, pb, ck, ua, c, ac, dh, dy, fD, kZ, bt, rA, ao, kV, gx, Gl, akd, aD, dM, ml, jh, ah, Gn, eq, Uy, ad, wD, dP, od, ds, io, hD, ji, uQ, gj, d, hG, ff, eH, cr, eZ, dN, Ej, aF, hp, Fq, Sm, iV, jb, bz, kN, aS, ra, lf, KS, X, hv); #c1(S100A8) c2(XP_011508163) c3(protein S100-A8 isoform X1) c4( ) c5(298676, 298677) c6( ) c7(oO, bM, T, ea, if, e, cl, ci, bh, Tz, ip, aE, dV, K, H, hH, eL, bc, aiJ, Us, hm, UC, li, yo, zQ, Jl, aW, lh, az, HA, kt, pD, aB, g, eE, eF, cm, y, eM, iT, dc, U, cj, uJ, Uz, eg, cE, dD, aw, Wb, aj, qN, kY, ai, h, dL, hB, pl, A, lb, fe, kT, dB, hk, hL, at, Br, uz, eo, Pr, B, it, L, bL, pF, mr, aA, cR, fF, v, j, C, AO, V, q, o, J, mj, lg, qp, ld, aT, cy, jj, kU, ae, dl, le, kl, s, Y, kX, fO, la, r, akY, lo, en, ik, pb, ck, ua, c, ac, dh, dy, fD, kZ, bt, rA, ao, kV, gx, Gl, akd, aD, dM, ml, jh, ah, Gn, eq, Uy, ad, wD, dP, od, ds, io, hD, ji, uQ, gj, d, hG, ff, eH, cr, eZ, dN, Ej, aF, hp, Fq, Sm, iV, jb, bz, kN, aS, ra, lf, kS, X, hv); #c1(S100A9) c2(NP_002956) c3(protein S100-A9) c4(133919) c5(133920, 133921, 133922, 133923, 133924, 133925) c6( ) c7(T, ea, if, e, cL, ci, bh, ip, x, afr, aE, bW, dV, rB, K, bah, hu, H, hH, eL, bc, Us, hm, UC, yo, aW, az, HA, pD, g, as, pL, eE, cm, bw, y, eM, U, cj, uJ, k, Uz, eg, Vj, iu, sc, dD, aEj, aw, ht, n, aj, fr, qN, ai, Bl, ft, h, dL, wb, A, fe, aq, ar, dB, mg, at, eo, zB, Pr, B, L, bL, vQ, pF, eR, aA, cR, fF, v, j, C, AO, V, pd, q, o, J, eA, aT, cy, ae, af, jf, kl, s, Y, kX, fO, r, lo, Xk, en, pb, ck, ua, c, ac, dh, mf, EY, fD, ao, gx, aD, dM, jh, ah, fu, eq, Uy, ad, wD, dP, fU, F, hD, ji, uQ, gj, d, ff, cr, eZ, dN, aF, hp, Fq, iV, xZ, aS, lf, ra, X, hv); #c1(S100B) c2(NP_006263) c3(protein S100-B) c4(163393) c5(163394, 163395, 163396, 163397, 163398, 163399, 163400, 163401, 163402, 163403) c6( ) c7(bM, T, aNA, Tb, fR, apr, ea, chc, bO, eb, gA, ci, mF, ip, azY, wM, aE, Bc, iX, aeO, SV, Ou, K, H, eL, cp, yo, aeR, aW, Ls, ih, g, as, eF, cm, uC, mZ, eM, iA, ki, cAS, U, Qw, PV, mk, yA, R, dD, aHS, aw, n, aj, ct, aBl, cd, ai, eW, Bl, cc, acA, KH, h, lG, SH, dL, mV, mY, he, dx, Yl, Lg, mU, kj, fe, aq, kp, mX, hq, cA, aEG, anx, agy, it, B, mT, L, ka, aoU, jx, aA, cR, mS, hz, Kh, aql, V, avD, q, J, eA, aaF, acj, hb, qK, aT, cy, bK, bq, avt, ae, af, qk, eu, jf, iF, cAR, Y, aPf, lo, Xk, f, acW, Dy, ac, iM, il, wR, CZ, auN, fD, aps, Kd, id, atY, aaz, aPJ, ha, rl, gN, jh, bS, eq, qi, ad, iC, Tg, Rq, mW, Ot, hD, hP, fb, atX, d, je, afe, eH, cr, rM, eZ, asV, aF, hp, aiz, iV, Z, adj, es, bhO, Ew, VO, MJ, aS, y, xg, iu); #c1(S100P) c2(NP_005971) c3(protein S100-P) c4(160884) c5(160885, 160886, 160887, 160888, 160889, 160890) c6( ) c7(T, aC, id, aA, bB, fr, ci, qN, q, ad, eJ, dP, jz, aE, aT, aU, ae, aEL, kl, B, s, xR, aW, r, fe, aq, kp, aF, g, iV, iR, t, es, eM, bE, PV, m, it, X); #c1(S100Z) c2(NP_570128) c3(protein S100-Z) c4(234014) c5(234015, 234016, 234017, 234018, 234019, 234020, 234021, 234022, 234023, 234024) c6( ) c7(IV); #c1(SIPR2) c2(NP_004221) c3(sphingosine 1-phosphate receptor 2) c4(144406) c5(144407, 144408, 144409, 144410, 144411, 144412, 144413, 144414, 144415, 144416) c6( ) c7(dP, dD, aW, dh, al, gZ, cj, aS, K, eq, bq, y, eA); #c1(SIPR3) c2(NP_005217) c3(sphingosine 1-phosphate receptor 3) c4(154105) c5(154106, 154107, 154108, 154109, 154110, 154111, 154112, 154113, 154114, 154115) c6( ) c7(iC, T, aW, aT, fF, cy, n, bnt, C, y); #c1(SIPR4) c2(NP_003766) c3(sphingosine 1-phosphate receptor 4 precursor) c4(140671) c5(140672, 140673, 140674, 140675, 140676, 140677) c6( ) c7(aW, T, aT, bq); #c1(SIPR5) c2(NP_001159687) c3(sphingosine 1-phosphate receptor 5) c4(64582, 64583) c5(64584, 64585, 64586, 64587, 64588, 64589, 64590, 64591, 64592, 64593, 64594, 64595, 64596, 64597, 64598, 64599) c6( ) c7(ck, d, v, K, bq, ae, ad); #c1(SAA1) c2(NP_001171477) c3(serum amyloid A-1 protein preproprotein) c4(70271) c5(70272, 70273, 70274, 70275, 70276, 70277, 70278, 70279, 70280, 70281, 70282, 70283, 70284, 70285) c6( ) c7(oO, T, dD, fR, Ke, ea, e, qx, jx, cz, bB, cL, v, j, oZ, eq, ai, o, nH, sO, ql, ja, cU, K, dQ, H, af, aGL, B, mR, fO, ih, cr, fc, iV, dw, YZ, cm, bw, bE, vZ, R, Lz, pJ, X, y); #c1(SAA2) c2(NP_001120852) c3(serum amyloid A-2 protein isoform b precursor) c4(44844) c5(44845, 44846, 44847, 44848, 44849, 44850) c6( ) c7(Ke, e, ih, R, Lz, dw, he, af, y); #c1(SAA2) c2(NP_110381) c3(serum amyloid A-2 protein isoform a precursor) c4(219819) c5(219820, 219821, 219822, 219823, 219824, 219825, 219826, 219827, 219828, 219829, 219830, 219831, 219832, 219833) c6( ) c7(Ke, e, ih, R, Lz, dw, he, af, y); #c1(SAA4) c2(NP_006503) c3(serum amyloid A-4 protein precursor) c4( ) c5(165863, 165864, 165865, 165866, 165867) c6( ) c7(Ke, eH, fZ, j, Lz, eF); #c1(SAAL1) c2(NP_612430) c3(protein SAAL1) c4(236442) c5(236443, 236444, 236445, 236446, 236447, 236448, 236449, 236450) c6(236451, 236452) c7(y, xE, zr); #c1(SAC3D1) c2(NP_037431) c3(SAC3 domain-containing protein 1) c4(175497, 175498) c5(175499, 175500, 175501, 175502, 175503, 175504, 175505, 175506, 175507, 175508, 175509, 175510, 175511, 175512, 175513, 175514) c6( ) c7(bB, bE, Y); #c1(SACM1L) c2(NP_054735) c3(phosphatidylinositide phosphatase SAC1) c4(177472) c5(177473, 177474, 177475, 177476, 177477, 177478) c6( ) c7(bL, ck, x, wg, ac, iM, je, aW, aT, kH, vr, aj, dy, k, Pd, y, J, tD, Rf); #c1(SACM1L) c2(XP_011531802) c3 (phosphatidylinositide phosphatase SAC1 isoform X1) c4(327881) c5(327882, 327883, 327884, 327885, 327886, 327887) c6( ) c7(bL, ck, x, wg, ac, iM, je, aW, aT, kH, vr, aj, dy, k, Pd, y, J, tD, Rf); #c1(SACS) c2(NP_001264984) c3(sacsin isoform 2) c4(97667) c5( ) c6( ) c7(ky, ads, KB, KH, U, ao, yZ, fu, bX, yU, gT, ado, aR); #c1(SACS) c2(NP_055178) c3(sacsin isoform 1) c4(179593) c5(179594, 179595, 179596, 179597, 179598, 179599, 179600, 179601, 179602, 179603, 179604) c6( ) c7(ky, ads, KB, KH, U, ao, yZ, fu, bX, yU, gT, ado, aR); #c1(SACS) c2(XP_011533341) c3(sacsin isoform X3) c4( ) c5(329465, 329466, 329467, 329468, 329469) c6( ) c7(ky, ads, kB, KH, U, ao, yZ, fu, bX, yU, gT, ado, aR); #c1(SAE1) c2(NP_001139185) c3(SUMO-activating enzyme subunit 1 isoform b) c4(58871) c5(58872, 58873, 58874, 58875, 58876, 58877) c6( ) c7(V, aT); #c1(SAE1) c2(NP_001139186) c3(SUMO-activating enzyme subunit 1 isoform c) c4(58878) c5(58879, 58880, 58881, 58882, 58883, 58884) c6( ) c7(V, aT); #c1(SAE1) c2(NP_005491) c3(SUMO-activating enzyme subunit 1 isoform a) c4(156759, 156760) c5(156761, 156762, 156763, 156764, 156765, 156766, 156767, 156768, 156769, 156770, 156771, 156772) c6( ) c7(V, aT); #c1(SAE1) c2(XP_011524607) c3(SUMO-activating enzyme subunit 1 isoform X2) c4(319150) c5(319151, 319152, 319153, 319154, 319155, 319156) c6( ) c7(V, aT); #c1(SAFB) c2(NP_001188267) c3(scaffold attachment factor BI isoform 1) c4(78981) c5( ) c6( ) c7(T, iH, akc, pf, if, qN, jz, ds, al, aPe, K, oi, aW, ff, Rr, LZ, gl, en, g, el, fy, cE); #c1(SAFB) c2(NP_001188268) c3(scaffold attachment factor B1 isoform 2) c4(78982) c5( ) c6( ) c7(T, iH, akc, pf, if, qN, jz, ds, aT, aPe, K, oi, aW, ff, Rr, LZ, gl, en, g, el, fy, cE); #c1(SAFB) c2(NP_00188269) c3(scaffold attachment factor B1 isoform 4) c4(78983, 78984, 78985) c5(78986, 78987, 78988, 78989, 78990, 78991, 78992, 78993, 78994, 78995, 78996, 78997, 78998, 78999, 79000, 79001, 79002, 79003, 79004, 79005, 79006, 79007, 79008, 79009) c6( ) c7(T, iH, akc, pf, if, qN, jz, ds, aT, aPe, K, oi, aW, ff, Rr, LZ, gl, en, g, el, fy, cE); #c1(SAFB) c2(NP_002958) c3(scaffold attachment factor B1 isoform 3) c4(133937) c5( ) c6( ) c7(T, iH, akc, pf, if, qN, jz, ds, aT, aPe, K, oi, aW, ff, Rr, LZ, gl, en, g, el, fy, cE); #c1(SAFB) c2(XP_006722902) c3(scaffold attachment factor B1 isoform X1) c4( ) c5(294927, 294928, 294929, 294930, 294931, 294932, 294933, 294934, 294935, 294936, 294937, 294938, 294939) c6( ) c7(T, iH, akc, pf, if, qN, jz, ds, aT, aPe, K, oi, aW, ff, Rr, LZ, gl, en, g, el, fy, cE); #c1(SAFB2) c2(NP_055464) c3(scaffold attachment factor 82) c4(181329, 181330, 181331) c5(181332, 181333, 181334, 181335, 181336, 181337, 181338) c6( ) c7(T, K, aT); #c1(SAFB2) c2(XP_011526751) c3(scaffold attachment factor B2 isoform X1) c4(321045) c5(321046, 321047, 321048, 321049, 321050, 321051, 321052, 321053, 321054, 321055, 321056, 321057, 321058, 321059, 321060, 321061, 321062) c6( ) c7(T, K, aT); #c1(SAFB2) c2(XP_011526752) c3(scaffold attachment factor B2 isoform X2) c4( ) c5(321063) c6( ) c7(T, K, aT); #c1(SAG) c2(NP_000532) c3(S-arrestin) c4(12041, 12042, 12043, 12044) c5(12045, 12046, 12047, 12048, 12049, 12050, 12051, 12052, 12053, 12054, 12055, 12056, (2057) c6( ) c7(tp, LX, e, cL, qW, o, ds, cy, K, hH, A, lR, cAU, bgh, fe, nk, nC, ayj, Dm, bgg, chr, bE, qr, agl, lf, LT); #c1(SAG) c2(XP_011547162) c3(S-arrestin isoform X3) c4( ) c5(345899) c6( ) c7(tp, LX, e, cL, qW, o, ds, cy, K, hH, A, lR, cAU, bgh, fe, nk, nC, ayj, Dm, bgg, chr, bE, qr, agl, lf, LT); #c1(SAG) c2(XP_011547165) c3(S-arrestin isoform X6) c4( ) c5(345900, 345901, 345902, 345903, 345904, 345905, 345906, 345907, 345908, 345909, 345910) c6( ) c7(tp, LX, e, cL, qW, o, ds, cy, K, hH, A, lR, cAU, bgh, fe, nk, nC, ayj, Dm, bgg, chr, bE, qr, agl, lf, LT); #c1(SAG) c2(XP_011547167) c3(S-arrestin isoform X7) c4(345911) c5(345912, 345913, 345914, 345915, 345916) c6( ) c7(tp, LX, e, cL, qW, o, ds, cy, K, hH, A, lR, cAU, bgh, fe, nk, nC, ayj, Dm, bgg, chr, bE, qr, agl, lf, LT); #c1(SAGE1) c2(NP_061136) c3(sarcoma antigen 1) c4(199460) c5(199461, 199462, 199463, 199464, 199465, 199466, 199467, 199468, 199469, 199470, 199471, 199472, 199473, 199474, 199475, 199476, 199477, 199478, 199479, 199480, 199481, 199482, 199483) c6( ) c7(bL, bM, T, wu, NT, aA, n, v, ci, ber, eA, ad, av, Bl, hd, aT, lQ, wm, an, ae, eL, jf, d, lR, aW, Y, Gv, ff, aF, en, bz, Hp, c, nB, eM, us); #c1(SAGE1) c2(XP_011529660) c3(sarcoma antigen 1 isoform X2) c4(325304) c5( ) c6( ) c7(bL, bM, T, wu, NT, aA, n, v, ci, ber, eA, ad, av, Bl, hd, aT, lQ, wm, an, ae, eL, jf, d, lR, aW, Y, Gv, ff, aF, en, bz, Hp, c, nB, eM, us); #c1(SALL1) c2(NP_001121364) c3(sal-like protein 1 isoform b) c4(45768, 45769, 45770) c5(45771, 45772, 45773, 45774, 45775, 45776) c6( ) c7(aMU, T, MS, yQ, op, aW, vA, aeq, aT, bko, dy, bkE, cAV, er, eW, atb); #c1(SALL1) c2(XP_011521557) c3(sal-like protein 1 isoform X1) c4(316272) c5(316273, 316274, 316275, 316276, 316277, 316278, 316279, 316280, 316281, 316282, 316283, 316284, 316285, 316286, 316287, 316288, 316289, 316290, 316291) c6( ) c7(aMU, T, MS, yQ, op, aW, vA, aeq, aT, bko, dy, bkE, cAV, er, eW, atb); #c1(SALL2) c2(NP_005398) c3(sal-like protein 2 isoform a) c4(155700, 155701) c5(155702, 155703, 155704, 155705, 155706, 155707, 155708) c6( ) c7(kn, d, oV, m, t, iJ, er); #c1(SALL2) c2(XP_011535366) c3(sal-like protein 2 isoform X1) c4( ) c5(332416, 332417, 332418, 332419, 332420, 332421) c6( ) c7(kn, d, oV, m, t, iJ, er); #c1(SALL3) c2(NP_741996) c3(sal-like protein 3) c4(247559, 247560, 247561) c5(247562, 247563, 247564, 247565, 247566, 247567, 247568, 247569, 247570, 247571, 247572, 247573, 247574, 247575, 247576, 247577, 247578, 247579, 247580, 247581, 247582) c6( ) c7(y, T, B, Az, pk); #c1(SALL4) c2(NP_065169) c3(sal-like protein 4) c4(204369) c5(204370, 204371, 204372, 204373, 204374, 204375, 204376) c6( ) c7(T, gU, atk, acl, cAW, aw, oA, aA, jh, hF, q, br, eA, x, hb, il, arH, aT, avQ, aW, oV, Az, r, aF, zZ, bw, ck, ou, aBU, cAX, aNy, k, bD, bt, xg, B); #c1(SALL4) c2(XP_011527224) c3(sal-like protein 4 isoform X2) c4(321772) c5( ) c6( ) c7(T, gU, atk, acl, cAW, aw, oA, aA, jh, hF, q, br, eA, x, hb, il, arH, aT, avQ, aW, oV, Az, r, af, zZ, bw, ck, ou, aBU, cAX, aNy, k, bD, bt, xg, B); #c1(SAMD1) c2(NP_612361) c3(atherin) c4( ) c5(236251, 236252, 236253, 236254, 236255) c6( ) c7(ea, dD, eq); #c1(SAMD14) c2(NP_777580) c3(sterile alpha motif domain-containing protein 14 isoform 1) c4(251252) c5(251253, 251254, 251255, 251256, 251257, 251258, 251259, 251260, 251261, 251262, 251263) c6( ) c7(zl, iu, g); #c1(SAMD14) c2(XP_006721816) c3(sterile alpha motif domain-containing protein 14 isoform X1) c4(294315) c5(294316, 294317, 294318, 294319, 294320, 294321) c6( ) c7(zl, iu, g); #c1(SAMD14) c2(XP_011522790) c3(sterile alpha motif domain-containing protein 14 isoform X2) c4( ) c5(317302) c6( ) c7(zl, iu, g); #c1(SAMD4A) c2(NP_001155048) c3(protein Smaug homolog 1 isoform 2) c4(61365, 61366) c5(61367, 61368, 61369, 61370, 61371, 61372, 61373, 61374, 61375, 61376, 61377, 61378) c6( ) c7(IB); #c1(SAMD4A) c2(NP_001155049) c3(protein Smaug homolog 1 isoform 3) c4(61379) c5(61380, 61381, 61382, 61383, 61384, 61385) c6( ) c7(IB); #c1(SAMD4A) c2(NP_056404) c3(protein Smaug homolog 1 isoform 1) c4(187432, 187433) c5(187434, 187435, 187436, 187437, 187438, 187439, 187440, 187441, 187442, 187443, 187444, 187445, 187446, 187447, 187448, 187449) c6( ) c7(IB); #c1(SAMD5) c2(NP_001025231) c3(sterile alpha motif domain-containing protein 5) c4(29528) c5(29529, 29530, 29531, 29532, 29533, 29534) c6( ) c7(af); #c1(SAMD9) c2(NP_060124) c3(sterile alpha motif domain-containing protein 9) c4(194289, 194290) c5(194291, 194292, 194293, 194294, 194295, 194296, 194297, 194298, 194299, 194300, 194301, 194302, 194303) c6( ) c7(T, dD, bE, ea, ds, cAY, bB, eq, bsR, lS); #c1(SAMD9L) c2(NP_001290426) c3(sterile alpha motif domain-containing protein 9-like) c4(115404, 115405, (15406) c5(115407, 115408, 115409, 115410, 115411, 115412, 115413, 115414, 115415, 115416, 115417, 115418, 115419, 115420, 115421, 115422, 115423, 115424, 115425, 115426, 115427, 115428, 115429, 115430, 115431, 115432, 115433, 115434, 115435, 115436, 115437, 115438, 115439, 115440, 115441, 115442, 115443, 115444) c6( ) c7(T); #c1(SAMHD1) c2(NP_056289) c3(deoxynucleoside triphosphate triphosphohydrolase SAMHD1) c4(186943, 186944) c5(186945, 186946, 186947, 186948, 186949, 186950) c6( ) c7(ck, eW, Ez, cAZ, ac, hb, eZ, cBa, ZQ, cj, lo, h, ey, gn, agy, eA, Ex, afu); #c1(SAMHD1) c2(XP_011527063) c3(deoxynucleoside triphosphate triphosphohydrolase SAMHD1 isoform X2) c4( ) c5(321501, 321502, 321503, 321504, 321505, 321506) c6( ) c7(ck, eW, Ez, cAZ, ac, hb, eZ, cBa, ZQ, cj, lo, h, ey, gn, agy, eA, Ex, afu); #c1(SAMM50) c2(NP_056195) c3(sorting and assembly machinery component 50 homolog) c4(186260, 186261) c5(186262, 186263, 186264, 186265, 186266, 186267, 186268, 186269, 186270, 186271, 186272, 186273) c6( ) c7(j); #c1(SAMSN1) c2(NP_001243299) c3(SAM domain-containing protein SAMSN-1 isoform 2) c4(90395) c5(90396, 90397, 90398, 90399, 90400, 90401) c6( ) c7(av, iV, d, oe, ey, hf, f, fq, eA); #c1(SAMSN1) c2(NP_001273452) c3(SAM domain-containing protein SAMSN-1 isoform 3) c4( ) c5(104931, 104932, 104933, 104934, 104935, 104936) c6( ) c7(av, iV, d, oe, ey, hf, f, fq, eA); #c1(SAMSN1) c2(NP_071419) c3(SAM domain-containing protein SAMSN-1 isoform 1) c4(211558) c5(211559, 211560, 211561, 211562, 211563, 211564) c6( ) c7(av, iV, d, oe, ey, hf, f, fq, eA); #c1(SAMSN1) c2(XP_011527986) c3(SAM domain-containing protein SAMSN-1 isoform X1) c4(323042) c5( ) c6( ) c7(av, iV, d, oe, ey, hf, f, fq, eA); #c1(SAP30BP) c2(NP_001288768) c3(SAP30-binding protein isoform 3) c4(114019) c5(114020, 114021, 114022, 114023, 114024, 114025) c6( ) c7(v, eA); #c1(SAP30BP) c2(NP_037392) c3(SAP30-binding protein isoform 2) c4(175209, 175210) c5(175211, 175212, 175213, 175214, 175215, 175216, 175217, 175218, 175219, 175220, 175221, 175222, 175223, 175224, 175225, (75226) c6( ) c7(v, eA); #c1(SAP30L) c2(NP_001124534) c3(histone deacetylase complex subunit SAP30L isoform 2) c4(49193) c5(49194, 49195, 49196, 49197, 49198, 49199) c6( ) c7(aj, kl); #c1(SAP30L) c2(NP_001124535) c3(histone deacetylase complex subunit SAP30L isoform 3) c4(49200) c5(49201, 49202, 49203, 49204, 49205, 49206) c6( ) c7(aj, kl); #c1(SAP30L) c2(NP_078908) c3(histone deacetylase complex subunit SAP30L isoform 1) c4(216060) c5(216061, 216062, 216063, 216064, 216065, 216066) c6( ) c7(aj, kl); #c1(SAPCD1) c2(NP_001034740) c3(suppressor APC domain-containing protein 1) c4(32126, 32127, 32128) c5(32129, 32130, 32131, 32132, 32133, 32134, 32135, 32136, 32137, 32138, 32139, 32140, 32141, 32142, 32143, 32144, 32145, 32146, 32147, 32148, 32149, 32150, 32151, 32152, 32153, 32154, 32155, 32156, 32157, 32158, 32159, 32160, 32161, 32162, 32163, 32164, 32165, 32166, 32167, 32168, 32169, 32170, 32171, 32172, 32173, 32174, 32175, 32176, 32177, 32178, 32179, 32180, 32181, 32182, 32183, 32184, 32185, 32186, 32187, 32188, 32189, 32190, 32191) c6( ) c7(y, W, eZ); #c1(SAPCD2) c2(NP_848543) c3(suppressor APC domain-containing protein 2) c4(253799) c5(253800, 253801, 253802, 253803, 253804, 253805) c6( ) c7(aA, bE, aF, q, r); #c1(SAPCD2) c2(XP_011517482) c3(suppressor APC domain-containing protein 2 isoform X1) c4( ) c5(311232) c6( ) c7(aA, bE, aF, q, r); #c1(SARIA) c2(NP_001136120) c3(GTP-binding protein SAR1a) c4(54374, 54375, 54376, 54377, 54378) c5(54379, 54380, 54381, 54382, 54383, 54384, 54385, 54386, 54387, 54388, 54389, 54390, 54391, 54392, 54393, 54394, 54395, 54396, 54397, 54398, 54399, 54400, 54401, 54402, 54403, 54404, 54405, 54406, 54407, 54408, 54409, 54410, 54411, 54412, 54413, 54414, 54415, 54416, 54417, 54418, 54419, 54420, 54421, 54422, 54423, 54424) c6( ) c7(fM, qb, wO, akr, y, af, fi); #c1(SAR1B) c2(NP_001028675) c3(GTP-binding protein SAR1b) c4(30447, 30448, 30449, 30450) c5(30451, 30452, 30453, 30454, 30455, 30456, 30457, 30458, 30459, 30460, 30461, 30462, 30463, 30464, 30465, 30466, 30467, 30468, 30469, 30470, 30471, 30472, 30473, 30474, 30475, 30476, 30477, 30478, 30479, 30480, 30481, 30482, 30483, 30484, 30485, 30486, 30487, 30488, 30489, 30490) c6( ) c7(bD, ZU, ZV, fi, C); #c1(SARDH) c2(NP_001128179) c3(sarcosine dehydrogenase, mitochondrial precursor) c4(49642, 49643, 49644, 49645, 49646, 49647, 49648, 49649, 49650) c5(49651, 49652, 49653, 49654, 49655, 49656, 49657, 49658, 49659, 49660, 49661, 49662, 49663, 49664, 49665, 49666, 49667, 49668, 49669, 49670, 49671, 49672, 49673, 49674, 49675, 49676, 49677, 49678, 49679, 49680, 49681, 49682, 49683, 49684) c6( ) c7(bL, T, cz, cBb, dG, fr, ci, eA, ad, cc, vz, ds, aT, h, bj, dL, K, vx, fb, ae, eL, yo, aW, dg, kC, op, ck, es, Ew, iA, lp, vA, vy, gv, anx, agy, y, VB); #c1(SARDH) c2(XP_011516635) c3(sarcosine dehydrogenase, mitochondrial isoform X2) c4( ) c5(310046, 310047, 310048, 310049, 310050, 310051, 310052, 310053, 310054, 310055, 310056, 310057, 310058, 310059, 310060, 310061, 310062, 310063, 310064, 310065, 310066, 310067, 310068, 310069) c6( ) c7(bL, T, cz, cBb, dG, fr, ci, eA, ad, cc, vz, ds, aT, h, bj, dL, K, vx, fb, ae, eL, yo, aW, dg, kC, op, ck, es, Ew, iA, lp, vA, vy, gv, anx, agy, y, VB); #c1(SARM1) c2(NP_055892) c3(sterile alpha and TIR motif-containing protein 1 precursor) c4(184229, 184230) c5(184231, 184232, 184233, 184234, 184235, 184236, 184237, 184238, 184239, 184240, 184241, 184242, 184243, 184244, 184245, 184246) c6( ) c7(Dw, cch, fM, ds, jh, cU, K); #c1(SARNP) c2(NP_149073) c3(SAP domain-containing ribonucleoprotein) c4(226995, 226996) c5(226997, 226998, 226999, 227000, 227001, 227002, 227003, 227004, 227005, 227006, 227007, 227008, 227009, 227010, 227011, 227012) c6( ) c7(gk, T, eJ, eM, eR, al, hF, bz, B, hX); #c1(SARS2) c2(NP_001139373) c3(serine-tRNA ligase, mitochondrial isoform a precursor) c4(59216) c5(59217, 59218, 59219, 59220, 59221, 59222, 59223, 59224, 59225, 59226) c6( ) c7(DS, fG, aw, cBc, fF, auq, aNG, fw, nH, afm, hb, ds, dk, aT, h, ccl, aa, afu, aW, wc, apn, sP, Ni, aiT, pR, lo, aq, CQ, TA, bE, uR, Ei, yH, fD, hE, B); #c1(SARS2) c2(NP_060297) c3(serine-tRNA ligase, mitochondrial isoform b precursor) c4(195252, 195253, 195254) c5(195255, 195256, 195257, 195258, 195259, 195260, 195261, 195262, 195263, 195264, 195265, 195266, 195267, 195268, 195269, 195270, 195271, 195272) c6( ) c7(DS, fG, aw, cBc, fF, auq, aNG, fw, nH, afm, hb, ds, dk, aT, h, ccl, aa, afu, aW, wc, apn, sP, Ni, aiT, pR, lo, aq, CQ, TA, bE, uR, Ei, yH, fD, hE, B); #c1(SART1) c2(NP_005137) c3(U4/U6.U5 tri-snRNP-associated protein 1) c4(153309, 153310) c5(153311, 153312, 153313, 153314, 153315, 153316, 153317, 153318, 153319, 153320, 153321, 153322) c6( ) c7(T, d, aW, ff, r, aT, en, q, ad); #c1(SART3) c2(NP_055521) c3(squamous cell carcinoma antigen recognized by T-cells 3) c4(181705, 181706, 181707, 181708, 181709) c5(181710, 181711, 181712, 181713, 181714, 181715, 181716, 181717, 181718, 181719, 181720, 181721, 181722, 181723, 181724, 181725, 181726, 181727, 181728, 181729, 181730, 181731, 181732, 181733, 181734, 181735) c6( ) c7(bM, cZ, T, wu, jh, fd, bB, agH, qN, allo, ad, kH, wm, dd, zy, eL, ae, je, aW, cm, t, LB, iM, ahC, m); #c1(SART3) c2(XP_011537328) c3(squamous cell carcinoma antigen recognized by T-cells 3 isoform X2) c4( ) c5(335034, 335035, 335036, 335037, 335038) c6( ) c7(bM, cZ, T, wu, jh, fd, bB, agH, qN, allo, ad, kH, wm, dd, zy, eL, ae, je, aW, cm, t, LB, iM, ahC, m); #c1(SASH1) c2(NP_056093) c3(SAM and SH3 domain-containing protein 1) c4(185679) c5(185680, 185681, 185682, 185683, 185684, 185685, 185686, 185687, 185688, 185689, 185690, 185691) c6( ) c7(T, af, bE, aW, aT, bB, n, aeM, bz, X); #c1(SASH1) c2(XP_005266932) c3(SAM and SH3 domain-containing protein 1 isoform X7) c4(280413) c5(280414, 280415, 280416, 280417, 280418, 280419) c6( ) c7(T, af, bE, aW, aT, bB, n, aeM, bz, X); #c1(SASH1) c2(XP_011533941) c3(SAM and SH3 domain-containing protein 1 isoform X4) c4(330130) c5( ) c6( ) c7(T, af, bE, aW, aT, bB, n, aeM, bz, X); #c1(SASH1) c2(XP_011533942) c3(SAM and SH3 domain-containing protein 1 isoform X5) c4(330131, 330132) c5(330133, 330134, 330135, 330136, 330137, 330138, 330139, 330140, 330141, 330142, 330143, 330144) c6( ) c7(T, af, bE, aW, aT, bB, n, aeM, bz, X); #c1(SASH3) c2(NP_061863) c3(SAM and SH3 domain-containing protein 3) c4(200847) c5(200848, 200849, 200850, 200851, 200852, 200853) c6( ) c7(gZ, bB, bE); #c1(SASH3) c2(XP_006724826) c3(SAM and SH3 domain-containing protein 3 isoform X1) c4(296445) c5(296446, 296447, 296448, 296449, 296450, 296451) c6( ) c7(gZ, bB, bE); #c1(SAT1) c2(NP_002961) c3(diamine acetyltransferase 1) c4(133945, 133946, 133947, 133948, 133949, 133950, 133951) c5(133952, 133953, 133954, 133955, 133956, 133957, 133958, 133959, 133960, 133961, 133962, 133963, 133964, 133965, 133966, 133967, 133968, 133969, 133970, 133971, 133972, 133973, 133974, 133975, 133976, 133977, 133978, 133979, 133980, 133981) c6( ) c7(bL, NK, T, dD, ea, NT, bO, aA, bB, fd, jh, bS, j, ct, v, eq, Jt, q, cd, ad, akE, av, ft, bgm, aT, lG, bK, K, bUo, ey, he, ae, eL, B, bkf, s, aW, Y, fJ, r, aq, aF, iV, Z, t, bz, GR, hq, vK, amS, cA, ig, bE, eM, MB, m, ei, y, X); #c1(SAT2) c2(NP_597998) c3 (diamine acetyltransferase 2) c4(235236, 235237) c5(235238, 235239, 235240, 235241, 235242, 235243, 235244, 235245, 235246, 235247, 235248, 235249) c6( ) c7(T, fR, s, aW, Y, aT, aA, LA, at, dL, ci, Dz, g, B, Qe); #c1(SATB1) c2(NP_001182399) c3 (DNA-binding protein SATB1 isoform 2) c4(74875, 74876) c5(74877, 74878, 74879, 74880, 74881, 74882) c6( ) c7(T, AY, eX, NT, jh, aA, fr, ci, q, ad, eA, b, aT, eV, dL, ae, eL, d, aW, ff, cr, r, fe, aq, aF, PQ, en, iR, bz, fs, bP, bE, at, i, bt, B); #c1(SATB1) c2(NP_002962) c3(DNA-binding protein SATB1 isoform 1) c4(133982, 133983, 133984, 133985, 133986) c5(133987, 133988, 133989, 133990, 133991, 133992, 133993, 133994, 133995, 133996, 133997) c6( ) c7(T, AY, eX, NT, jh, aA, fr, ci, q, ad, eA, b, aT, eV, dL, ae, eL, d, aW, ff, cr, r, fe, aq, af, PQ, en, iR, bz, fs, bP, bE, at, i, bt, B); #c1(SATB1) c2(XP_011532292) c3(DNA-binding protein SATB1 isoform X2) c4( ) c5(328403, 328404, 328405, 328406, 328407, 328408, 328409, 328410, 328411, 328412, 328413, 328414, 328415, 328416, 328417, 328418, 328419, 328420, 328421, 328422, 328423, 328424, 328425, 328426, 328427, 328428, 328429, 328430, 328431, 328432) c6( ) c7(T, AY, eX, NT, jh, aA, fr, ci, q, ad, eA, b, aT, eV, dL, ae, eL, d, aW, ff, cr, r, fe, aq, af, PQ, en, iR, bz, fs, bP, bE, at, i, bt, B); #c1(SATB2) c2(XP_005246453) c3(DNA-binding protein SATB2 isoform X2) c4(267149) c5(267150, 267151, 267152, 267153, 267154, 267155) c6( ) c7(cBe, T, oe, ci, nA, q, Na, ft, cBf, cBg, rz, eH, Az, r, Ej, cl, eF, iV, iK, QG, cBd, hL, zu, rA); #c1(SATB2) c2(XP_011509142) c3(DNA-binding protein SATB2 isoform X1) c4(299763) c5(299764, 299765, 299766, 299767, 299768, 299769, 299770, 299771, 299772, 299773, 299774, 299775, 299776, 299777, 299778) c6( ) c7(cBe, T, oe, ci, nA, q, Na, ft, cBf, cBg, rz, eH, Az, r, Ej, cl, eF, iV, ik, QG, cBd, hL, zu, rA); #c1(SATL1) c2(NP_001012998) c3(spermidine/spermine N(1)-acetyltransferase-like protein 1) c4(25415, 25416) c5(25417, 25418, 25419, 25420, 25421, 25422, 25423, 25424, 25425, 25426, 25427, 25428) c6( ) c7(c); #c1(SATL1) c2(XP_011529242) c3(spermidine/spermine N(1)-acetyltransferase-like protein 1 isoform X1) c4(324679) c5(324680, 324681, 324682, 324683, 324684, 324685) c6( ) c7(c); #c1(SAV1) c2(NP_068590) c3(protein salvador homolog 1) c4(209718, 209719) c5(209720, 209721, 209722, 209723, 209724, 209725, 209726, 209727, 209728) c6( ) c7(T, cd, py); #c1(SAV1) c2(XP_011535359) c3(protein salvador homolog 1 isoform X1) c4( ) c5(332410, 332411, 332412, 332413, 332414, 332415) c6( ) c7(T, cd, py); #c1(SBDS) c2(NP_057 (22) c3(ribosome maturation protein SBDS) c4(188995) c5(188996, 188997, 188998, 188999, 189000, 189001) c6( ) c7(bL, gV, T, aZA, aFQ, if, Am, fd, jh, bs, bBX, cz, afK, bS, j, ct, gf, aVK, v, bB, oY, qN, lR, br, ad, wD, mj, b, CJ, oz, vJ, ds, i, dv, By, K, aKG, H, ae, af, qk, bA, asA, sb, akV, arl, A, hG, Cw, Fm, Y, az, Rj, hl, eZ, ff, hp, en, g, Z, bw, qs, pb, Wt, yd, ck, VJ, bE, U, cj, dW, XO, hh, y, bt, ao, L); #c1(SBF1) c2(NP_002963) c3(myotubularin-related protein 5) c4(133998, 133999) c5(134000, 134001, 134002, 134003, 134004, 134005, 134006, 134007, 134008, 134009, 134010, 134011) c6( ) c7(gU, cBh, ac, ND, Y, bbH, vH, cgl, bU, bX, dr); #c1(SBF1) c2(XP_005261988) c3(myotubularin-related protein 5 isoform X6) c4(277684) c5(277685, 277686, 277687, 277688, 277689, 277690) c6( ) c7(gU, cBh, ac, ND, Y, bbH, vH, cgl, bu, bX, dr); #c1(SBF1) c2(XP_005261992) c3(myotubularin-related protein 5 isoform X7) c4(277691) c5(277692, 277693, 277694, 277695, 277696, 277697) c6( ) c7(gU, cBh, ac, ND, Y, bbH, vH, cgl, bU, bX, dr); #c1(SBF1) c2(XP_011529011) c3(myotubularin-related protein 5 isoform X3) c4( ) c5(324374) c6( ) c7(gU, cBh, ac, ND, Y, bbH, vH, cgl, bu, bX, dr); #c1(SBF2) c2(NP_112224) c3(myotubularin-related protein 13) c4(220964) c5(220965, 220966, 220967, 220968, 220969, 220970) c6( ) c7(DD, cBi, agU, xz, cgJ, bbH, vr, cgl, bU, dw, bX); #c1(SBN01) c2(NP_001161328) c3(protein strawberry notch homolog I isoform 1) c4(65603) c5(65604, 65605, 65606, 65607, 65608, 65609) c6( ) c7(hb, ci, eM, Os); #c1(SBN01) c2(NP_060653) c3(protein strawberry notch homolog 1 isoform 2) c4(197008, 197009) c5(197010, 197011, 197012, 197013, 197014, 197015, 197016, 197017, 197018, 197019, 197020, 197021) c6( ) c7(hb, ci, eM, Os); #c1(SBN02) c2(NP_001093592) c3(protein strawberry notch homolog 2 isoform 2) c4(40332, 40333) c5(40334, 40335, 40336, 40337, 40338, 40339, 40340, 40341, 40342, 40343, 40344, 40345) c6( ) c7(fe, iV, ci, eM, MC); #c1(SBN02) c2(NP_055778) c3(protein strawberry notch homolog 2 isoform 1) c4(183456) c5(183457, 183458, 183459, 183460, 183461, 183462) c6( ) c7(fe, iV, ci, eM, MC); #c1(SBSN) c2(NP_001159506) c3(suprabasin isoform 1 precursor) c4(64177) c5(64178, 64179, 64180, 64181, 64182, 64183) c6( ) c7(bz, qK); #c1(SBSN) c2(NP_001159507) c3(suprabasin isoform 3 precursor) c4(64184) c5(64185, 64186, 64187, 64188, 64189, 64190) c6( ) c7(bz, qK); #c1(SBSN) c2(NP_940940) c3(suprabasin isoform 2 precursor) c4(260151) c5(260152, 260153, 260154, 260155, 260156, 260157) c6( ) c7(bz, qK); #c1(SC5D) c2(NP_008849) c3(lathosterol oxidase) c4(169099) c5(169100, 169101, 169102, 169103, 169104, 169105, 169106, 169107, 169108, 169109) c6( ) c7(Na, ac, hl, nA, y, beW, ae, ad); #c1(SCAF1) c2(NP_067051) c3(splicing factor, arginine/serine-rich 19) c4(208738, 208739) c5(208740, 208741, 208742, 208743, 208744, 208745, 208746, 208747, 208748, 208749, 208750, 208751) c6( ) c7(ck, gk, pr, bE, aW, aNd, axd, al, jx, aT, bB, m, jj, aNe, t, acD, kS, aR); #c1(SCAF1) c2(XP_011525496) c3(splicing factor, arginine/serine-rich 19 isoform X2) c4( ) c5(319889, 319890) c6( ) c7(ck, gk, pr, bE, aW, aNd, axd, al, jx, aT, bB, m, jj, aNe, t, acD, kS, aR); #c1(SCAF11) c2(NP_004710) c3(protein SCAF11) c4( ) c5(148997, 148998, 148999, 149000, 149001, 149002, 149003, 149004, 149005, 149006, 149007, 149008, 149009, 149010) c6( ) c7(T, aAf, Ol, aA, bB, fr, ci, eA, aE, Tf, h, kf, ahE, ff, aq, aF, ahL, en, ik, eM, bE, m, B, hS); #c1(SCAF11) c2(XP_006719755) c3(protein SCAF11 isoform X5) c4(292965) c5( ) c6( ) c7(T, aAf, Ol, aA, bB, fr, ci, eA, aE, Tf, h, kf, ahE, ff, aq, aF, ahL, en, ik, eM, bE, m, B, hS); #c1(SCAF11) c2(XP_011537286) c3 (protein SCAF11 isoform X3) c4(334999) c5(335000, 335001, 335002) c6( ) c7(T, aAf, Ol, aA, bB, fr, ci, eA, aE, Tf, h, kf, ahE, ff, aq, aF, ahL, en, ik, eM, bE, m, B, hS); #c1(SCAF4) c2(NP_001138916) c3(splicing factor, arginine/serine-rich 15 isoform 2) c4(58187) c5(58188, 58189, 58190, 58191, 58192, 58193) c6( ) c7(aj, J, vy, sj); #c1(SCAF4) c2(NP_001138917) c3(splicing factor, arginine/serine-rich 15 isoform 3) c4(58194) c5(58195, 58196, 58197, 58198, 58199, 58200, 58201, 58202, 58203, 58204, 58205, 58206) c6( ) c7(aj, J, vy, sj); #c1(SCAF4) c2(NP_065757) c3(splicing factor, arginine/serine-rich 15 isoform 1) c4(205216) c5( ) c6( ) c7(aj, J, vy, sj); #c1(SCAF4) c2(XP_005261074) c3(splicing factor, arginine/serine-rich 15 isoform X3) c4( ) c5(277133) c6( ) c7(aj, J, vy, sj); #c1(SCAF8) c2(NP_001273118) c3(protein SCAF8 isoform b) c4(104328) c5( ) c6( ) c7(bM, bK); #c1(SCAF8) c2(NP_055707) c3(protein SCAF8 isoform d) c4(182959) c5(182960, 182961, 182962, 182963, 182964, 182965, 182966, 182967, 182968, 182969, 182970, 182971, 182972, 182973, 182974, 182975) c6( ) c7(bM, bK); #c1(SCA1) c2(NP_001138349) c3(protein SCA1 isoform 2) c4(56241) c5(56242, 56243, 56244, 56245, 56246, 56247) c6( ) c7(aW, T, Hb, aT); #c1(SCA1) c2(NP_775961) c3(protein SCA1 isoform 1) c4(250838) c5(250839, 250840, 250841, 250842, 250843, 250844) c6( ) c7(aW, T, Hb, aT); #c1(SCAMP2) c2(NP_005688) c3(secretory carrier-associated membrane protein 2) c4(158572, 158573) c5(158574, 158575, 158576, 158577, 158578, 158579, 158580, 158581, 158582, 158583, 158584, 158585) c6( ) c7(FE, Jt); #c1(SCAMP5) c2(XP_006720483) c3(secretory carrier-associated membrane protein 5 isoform X2) c4(293524) c5(293525, 293526) c6( ) c7(bN); #c1(SCAMP5) c2(XP_011519618) c3(secretory carrier-associated membrane protein 5 isoform X1) c4( ) c5(314285, 314286, 314287, 314288, 314289, 314290, 314291, 314292) c6( ) c7(bN); #c1(SCAMP5) c2(XP_011519619) c3(secretory carrier-associated membrane protein 5 isoform X3) c4(314293) c5(314294, 314295, 314296, 314297, 314298, 314299) c6( ) c7(bN); #c1(SCAP) c2(NP_036367) c3(sterol regulatory element-binding protein cleavage-activating protein) c4(173275, 173276, 173277, (73278) c5(173279, 173280, 173281, 173282, 173283, 173284, 173285) c6( ) c7(dD, af, cH, ea, aw, gZ, Zv, v, j, K, eq, bq, dQ, dF, B); #c1(SCAP) c2(XP_005265024) c3(sterol regulatory element-binding protein cleavage-activating protein isoform X2) c4(279346, 279347, 279348) c5(279349, 279350, 279351, 279352, 279353, 279354, 279355, 279356, 279357, 279358, 279359, 279360, 279361, 279362, 279363, 279364, 279365, 279366, 279367, 279368, 279369, 279370, 279371, 279372, 279373) c6( ) c7(dD, af, cH, ea, aw, gZ, Zv, v, j, K, eq, bq, dQ, dF, B); #c1(SCAP) c2(XP_005265026) c3(sterol regulatory element-binding protein cleavage-activating protein isoform X4) c4(279374, 279375) c5(279376, 279377, 279378, 279379, 279380, 279381, 279382, 279383, 279384, 279385, 279386) c6( ) c7(dD, af, cH, ea, aw, gZ, Zv, v, j, K, eq, bq, dQ, dF, B); #c1(SCAP) c2(XP_005265029) c3(sterol regulatory element-binding protein cleavage-activating protein isoform X7) c4( ) c5(279387, 279388, 279389, 279390, 279391, 279392, 279393, 279394, 279395, 279396, 279397) c6( ) c7(dD, af, cH, ea, aw, gZ, Zv, v, j, K, eq, bq, dQ, dF, B); #c1(SCAPER) c2(NP_001139395) c3(S phase cyclin A-associated protein in the endoplasmic reticulum isoform b) c4(59281) c5(59282, 59283, 59284, 59285, 59286) c6( ) c7(Hr, zv, W, nA, iW); #c1(SCAPER) c2(NP_065894) c3(S phase cyclin A-associated protein in the endoplasmic reticulum isoform a) c4(206117) c5(206118, 206119, 206120, 206121, 206122, 206123, 206124, 206125, 206126, 206127, 206128) c6( ) c7(Hr, zv, W, nA, iW); #c1(SCAPER) c2(XP_011519950) c3(S phase cyclin A-associated protein in the endoplasmic reticulum isoform X2) c4( ) c5(314772) c6( ) c7(Hr, zv, W, nA, iW); #c1(SCAPER) c2(XP_011519954) c3(S phase cyclin A-associated protein in the endoplasmic reticulum isoform X6) c4(314773) c5(314774, 314775, 314776, 314777, 314778, 314779) c6( ) c7(Hr, zv, W, nA, iW); #c1(SCARA3) c2(NP_057324) c3(scavenger receptor class A member 3 isoform 1) c4(190142) c5(190143, 190144, 190145, 190146, 190147, 190148) c6( ) c7(ae, T, dD, ib, AD, aW, hT, Of, aT, fd, n, Og, eq, ci, K, m, ey, t, bz, ad); #c1(SCARA3) c2(NP_878185) c3(scavenger receptor class A member 3 isoform 2) c4(257049) c5(257050, 257051, 257052, 257053, 257054, 257055) c6( ) c7(ae, T, dD, ib, AO, aW, hT, Of, aT, fd, n, Og, eq, ci, K, m, ey, t, bz, ad); #c1(SCARA5) c2(NP_776194) c3(scavenger receptor class A member 5) c4(250970, 250971, 250972, 250973) c5(250974, 250975, 250976, 250977, 250978, 250979, 250980, 250981, 250982, 250983, 250984, 250985, 250986, 250987, 250988, 250989, 250990, 250991, 250992, 250993, 250994, 250995, 250996, 250997, 250998, 250999, 251000, 251001) c6( ) c7(T, B); #c1(SCARB1) c2(NP_001076428) c3(scavenger receptor class B member 1 isoform 2) c4(37390) c5(37391, 37392, 37393, 37394, 37395, 37396, 37397, 37398, 37399, 37400, 37401, 37402, 37403, 37404, 37405, 37406, 37407, 37408, 37409, 37410, 37411, 37412, 37413, 37414, 37415, 37416, 37417, 37418, 37419, 37420) c6( ) c7(bL, gh, T, dD, dA, fR, eR, ea, e, jx, aw, aj, v, j, ci, bX, eq, fr, zP, J, cBj, cF, Zu, aT, iv, h, dL, K, dQ, anN, aXa, af, zQ, s, cH, aW, ff, Bw, bbs, cu, Pp, en, jb, es, W, ky, gZ, at, oh, acl); #c1(SCARB1) c2(NP_005496) c3(scavenger receptor class B member 1 isoform 1) c4(156794, 156795, 156796, 156797, 156798, 156799, 156800) c5(156801, 156802, 156803, 156804, 156805, 156806, 156807, 156808, 156809, 156810, 156811, 156812, 156813, 156814, 156815, 156816, 156817, 156818) c6( ) c7(bL, gh, T, dD, dA, fR, eR, ea, e, jx, aw, aj, v, j, ci, bX, eq, fr, zP, J, cBj, cF, Zu, aT, iv, h, dL, K, da, anN, aXa, af, zQ, s, cH, aW, ff, Bw, bbs, cu, Pp, en, jb, es, W, ky, gZ, at, oh, acl); #c1(SCARB2) c2(NP_001191184) c3(lysosome membrane protein 2 isoform 2 precursor) c4(81094) c5(81095, 81096, 81097, 81098, 81099, 81100) c6( ) c7(vU, bM, yr, cc, agT, aYb, agU, ha, U, fP, cz, yZ, lo, ah, as, an, yU, aY, ae, cd, ad); #c1(SCARB2) c2(NP_005497) c3 (lysosome membrane protein 2 isoform 1 precursor) c4(156819) c5(156820, 156821, 156822, 156823, 156824, 156825) c6( ) c7(vU, bM, yr, cc, agT, aYb, agU, ha, U, fP, cz, yZ, lo, ah, as, an, yU, aY, ae, cd, ad); #c1(SCARF2) c2(NP_699165) c3(scavenger receptor class F member 2 isoform 1 precursor) c4( ) c5(245779, 245780, 245781, 245782, 245783, 245784) c6( ) c7(cBk, bX); #c1(SCARF2) c2(NP_878315) c3(scavenger receptor class F member 2 isoform 2 precursor) c4(257099, 257100, 257101) c5(257102, 257103, 257104, 257105, 257106, 257107, 257108, 257109, 257110, 257111, 257112, 257113, 257114, 257115, 257116, 257117, 257118, 257119, 257120, 257121) c6(257122, 257123, 257124) c7(cBk, bX); #c1(SCCPDH) c2(NP_057086) c3(saccharopine dehydrogenase-like oxidoreductase) c4(188862, 188863) c5(188864, 188865, 188866, 188867, 188868, 188869, 188870, 188871, 188872, 188873, 188874, 188875) c6( ) c7(aw); #c1(SCD) c2(NP_005054) c3(acyl-CoA desaturase) c4(152468, 152469) c5(152470, 152471, 152472, 152473, 152474, 152475, 152476, 152477, 152478, 152479, 152480, 152481) c6( ) c7(bL, T, dD, qb, ea, e, ew, aA, rf, n, aj, bS, j, ct, ci, v, eq, zP, Eh, Fb, J, ad, mP, fm, aT, jy, vc, bci, dL, K, qM, ae, af, HO, eL, mu, A, zQ, B, aW, fc, dq, aq, aF, g, du, Z, t, aot, bz, c, Fi, m, pw, oQ, ei, gq); #c1(SCD5) c2(NP_001032671) c3(stearoyl-CoA desaturase 5 isoform a) c4(31411) c5(31412, 31413, 31414, 31415, 31416, 31417) c6( ) c7(mR, oz, aw, eZ, h, ci, y, af); #c1(SCD5) c2(NP_079182) c3(stearoyl-CoA desaturase 5 isoform b) c4(217319, 217320) c5(217321, 217322, 217323, 217324, 217325, 217326, 217327, 217328, 217329, 217330, 217331, 217332) c6( ) c7(mR, oz, aw, eZ, h, ci, y, af); #c1(SCFD1) c2(NP_001244305) c3(sec1 family domain-containing protein 1 isoform c) c4(92115) c5( ) c6( ) c7(lG, T); #c1(SCFD1) c2(NP_001269961) c3(sec1 family domain-containing protein 1 isoform e) c4(103288) c5(103289, 103290, 103291, 103292, 103293, 103294) c6( ) c7(lG, T); #c1(SCFD1) c2(NP_057190) c3(sec1 family domain-containing protein 1 isoform a) c4(189356, 189357, 189358) c5(189359, 189360, 189361, 189362, 189363, 189364, 189365, 189366, 189367, 189368, 189369, 189370, 189371, 189372, 189373, 189374, 189375, 189376, 189377, 189378, 189379, 189380, 189381, 189382) c6( ) c7(lG, T); #c1(SCFD2) c2(NP_689753) c3(sec1 family domain-containing protein 2) c4(243859, 243860) c5(243861, 243862, 243863, 243864, 243865, 243866, 243867, 243868, 243869, 243870, 243871, 243872) c6( ) c7(fq, ajP, Ea, oQ); #c1(SCG2) c2(NP_003460) c3(secretogranin-2 precursor) c4(138484, 138485, 138486, 138487) c5(138488, 138489, 138490, 138491, 138492, 138493, 138494, 138495, 138496, 138497, 138498, 138499, 138500, 138501, 138502, 138503, 138504, 138505, 138506, 138507, 138508, 138509, 138510, 138511) c6( ) c7(T, qX, aw, cz, aj, j, V, rc, cd, J, pX, cy, K, dx, jf, iF, LC, Y, hp, g, cm, ac, c, y); #c1(SCG3) c2(NP_001158729) c3(secretogranin-3 isoform 2) c4(63736, 63737) c5(63738, 63739, 63740, 63741, 63742, 63743, 63744, 63745, 63746, 63747) c6( ) c7(bM, T, B, Uc, s, v, cm, af); #c1(SCG3) c2(NP_037375) c3(secretogranin-3 isoform 1 precursor) c4(175104) c5(175105, 175106, 175107, 175108, 175109, 175110, 175111, 175112) c6( ) c7(bM, T, B, Uc, s, v, cm, af); #c1(SCG5) c2(NP_001138229) c3(neuroendocrine protein 7B2 isoform 1 precursor) c4(56070) c5(56071, 56072, 56073, 56074, 56075, 56076, 56077, 56078) c6(56079, 56080) c7(jf, ft, AB, d, oe, qy, r, Ja, mf, m, bz, as, eA, t, cBl, q); #c1(SCG5) c2(NP_003011) c3(neuroendocrine protein 782 isoform 2 precursor) c4(134421) c5(134422, 134423, 134424, 134425, 134426, 134427, 134428, 134429) c6(134430, 134431) c7(jf, ft, AB, d, oe, qy, r, Ja, mf, m, bz, as, eA, t, cBl, q); #c1(SCGB1A1) c2(NP_003348) c3(uteroglobin precursor) c4(137694) c5(137695, 137696, 137697, 137698, 137699, 137700) c6( ) c7(bL, bM, T, Dw, JN, eR, fR, sQ, z, dM, fG, cz, fF, BH, j, ci, uE, zo, ad, Vs, av, cF, jz, aFh, apJ, aT, Ds, JK, ey, lQ, uN, aDZ, cBm, ae, eu, aa, uQ, s, aW, sp, mQ, eZ, g, t, cM, bz, baf, zV, DF, bwd, kT, cT, ac, JA, dB, hc, Ny, AM, Ei, hL, JM, uo, E, ut, uJ, dy, m, fD, at, y, qR, iu, aDj); #c1(SCGB1D1) c2(NP_006543) c3(secretoglobin family ID member 1 precursor) c4(166119) c5(166120, 166121, 166122, 166123, 166124, 166125, 166(26) c6(166127) c7(ej, W, e, aw, ff, cR, h, v, j, aEN, en, g, aKB, hX); #c1(SCGB1D2) c2(NP_006542) c3(secretoglobin family 1D member 2 precursor) c4(166110) c5(166111, 166112, 166113, 166114, 166115, 166116, 166117) c6(166118) c7(T, yo, aW, aT, cz, hR, ci, m, Dz, t); #c1(SCGB2A1) c2(NP_002398) c3(mammaglobin-B precursor) c4(128099) c5(128100, 128101, 128102, 128103, 128104, 128105) c6( ) c7(T, aA, bB, ci, q, jz, aT, MO, aW, Y, r, aq, aF, g, cl, t, bXl, bE, avk, hL, m, B); #c1(SCGB2A2) c2(NP 002402) c3(mammaglobin-A precursor) c4(128120) c5(128121, 128122, 128123, 128124, 128125, 128126) c6(128127) c7(av, T, W, aW, if, aT, n, ci, m, qN, g, t); #c1(SCGB2A2) c2(XP_005274062) c3(mammaglobin-A isoform X1) c4(284918) c5(284919, 284920, 284921, 284922, 284923, 284924, 284925, 284926, 284927) c6( ) c7(av, T, W, aW, if, al, n, ci, m, qN, g, t); #c1(SCGB2B2) c2(NP_001020762) c3(secretoglobin family 2B member 2 precursor) c4(28997) c5(28998, 28999, 29000, 29001, 29002, 29003, 29004, 29005, 29006, 29007) c6(29008, 29009) c7(oe); #c1(SCGB282) c2(XP_011525138) c3(secretoglobin family 28 member 2 isoform X1) c4( ) c5(319577, 319578) c6( ) c7(oe); #c1(SCGB3A1) c2(NP_443095) c3(secretoglobin family 3A member 1 precursor) c4(229377) c5(229378, 229379, 229380, 229381, 229382, 229383) c6( ) c7(T, jh, ci, ad, dP, bv, av, ft, Xc, ds, aT, s, aW, Lf, ff, en, bu, g, cm, bz, ck, bF, kP, eM, bP, bt); #c1(SCGB3A2) c2(NP_473364) c3(secretoglobin family 3A member 2 precursor) c4(230403) c5(230404, 230405, 230406, 230407, 230408, 230409) c6( ) c7(bM, z, fG, sp, aCx, hD, zV, fD, pM, ao); #c1(SCGN) c2(NP_008929) c3(secretagogin) c4(169613, 169614) c5(169615, 169616, 169617, 169618, 169619, 169620, 169621, 169622, 169623, 169624, 169625, 169626, 169627, 169628, 169629) c6(169630, 169631, 169632) c7(hR, yo, nn, q, r); #c1(SCHIP1) c2(NP_001184036) c3(schwannomin-interacting protein 1 isoform 2) c4(75292) c5(75293, 75294, 75295, 75296, 75297, 75298) c6( ) c7(bF, rQ, py, hp, rP); #c1(SCHIP1) c2(NP_001184037) c3(schwannomin-interacting protein 1 isoform 3) c4(75299) c5(75300, 75301, 75302, 75303, 75304, 75305) c6( ) c7(bF, rQ, py, hp, rP); #c1(SCHIP1) c2(NP_001184038) c3(schwannomin-interacting protein 1 isoform 4) c4(75306) c5(75307, 75308, 75309, 75310, 75311, 75312, 75313, 75314, 75315, 75316, 75317, 75318) c6( ) c7(bF, rQ, py, hp, rP); #c1(SCHIP1) c2(NP_055390) c3(schwannomin-interacting protein 1 isoform 1) c4(180739) c5(180740, 180741, 180742, 180743, 180744, 180745) c6( ) c7(bF, rQ, py, hp, rP); #c1(SCIN) c2(NP_001106177) c3(adseverin isoform 1) c4(42329, 42330) c5(42331, 42332, 42333, 42334, 42335, 42336, 42337, 42338, 42339, 42340, 42341, 42342, 42343, 42344, 42345, 42346) c6( ) c7(iY, ae, cd, ad); #c1(SCLT1) c2(NP_001287826) c3(sodium channel and clathrin linker 1 isoform 2) c4( ) c5(112931, 112932, 112933, 112934, 112935, 112936) c6( ) c7(AK, pi, Zf, bez, rS, dV, r, wR, vm, CZ, C, Za, q, ahD); #c1(SCLT1) c2(NP_001287827) c3(sodium channel and clathrin linker 1 isoform 3) c4( ) c5(112937, 112938, 112939, 112940, 112941, 112942) c6( ) c7(AK, pi, Zf, bez, rS, dV, r, wR, vm, CZ, C, Za, q, ahD); #c1(SCLT1) c2(NP_653244) c3(sodium channel and clathrin linker 1 isoform 1) c4(239302, 239303, 239304) c5(239305, 239306, 239307, 239308, 239309, 239310) c6( ) c7(AK, pi, Zf, bez, rS, dV, r, wR, vm, CZ, C, Za, q, ahD); #c1(SCLT1) c2(XP_011529894) c3(sodium channel and clathrin linker 1 isoform X1) c4(325657) c5( ) c6( ) c7(AK, pi, Zf, bez, rS, dV, r, wR, vm, CZ, C, Za, q, ahD); #c1(SCLT1) c2(XP_011529895) c3(sodium channel and clathrin linker 1 isoform X2) c4( ) c5(325658, 325659, 325660, 325661, 325662, 325663) c6( ) c7(AK, pi, Zf, bez, rS, dV, r, wR, vm, CZ, C, Za, q, ahD); #c1(SCLY) c2(NP_057594) c3(selenocysteine lyase) c4(191495, 191496, 191497, 191498, 191499, 191500) c5(191501, 191502, 191503, 191504, 191505, 191506, 191507, 191508, 191509, 191510, 191511, 191512, 191513, 191514, 191515, 191516, 191517, 191518, 191519, 191520, 191521, 191522, 191523, 191524, 191525, 191526, 191527, 191528, 191529, 191530, 191531, 191532, 191533, 191534, 191535, 191536) c6( ) c7(bdo, T, fM, dA, cbF, iW, oA, bcE, gf, bS, ct, ci, v, hF, aQF, wD, x, PW, qh, aql, kH, hn, K, bXV, iL, je, Y, ff, apK, en, Eg, Z, pb, hq, ck, rR, iM, iT, pg, dW, zv, k, adg, y, bt, B); #c1(SCML2) c2(NP_006080) c3(sex comb on midleg-like protein 2) c4(161751) c5(161752, 161753, 161754, 161755, 161756, 161757, 161758) c6( ) c7(jf); #c1(SCML2) c2(XP_006724521) c3(sex comb on midleg-like protein 2 isoform X3) c4(295990, 295991, 295992) c5(295993, 295994, 295995, 295996, 295997, 295998, 295999, 296000, 296001, 296002, 296003, 296004, 296005, 296006, 296007, 296008, 296009, 296010) c6( ) c7(jf); #c1(SCML4) c2(NP_932347) c3(sex comb on midleg-like protein 4 isoform a) c4(258973) c5(258974, 258975, 258976, 258977, 258978) c6( ) c7(fq); #c1(SCML4) c2(XP_011534003) c3(sex comb on midleg-like protein 4 isoform X6) c4( ) c5(330177, 330178) c6( ) c7(fq); #c1(SCML4) c2(XP_011534006) c3(sex comb on midleg-like protein 4 isoform X8) c4(330179) c5(330180, 330181, 330182, 330183, 330184, 330185) c6( ) c7(fq); #c1(SCN10A) c2(NP_006505) c3(sodium channel protein type 10 subunit alpha isoform 1) c4(165868) c5(165869, 165870, 165871, 165872, 165873, 165874) c6( ) c7(bM, cZ, cBn, T, akd, mr, aW, wL, My, cV, EQ, aT, ajM, xH, fu, dd, asT, cm, bt, cmk, aZQ); #c1(SCN11A) c2(XP_011531622) c3(sodium channel protein type 11 subunit alpha isoform X1) c4(327767, 327768) c5(327769, 327770, 327771, 327772, 327773, 327774, 327775, 327776, 327777, 327778, 327779, 327780, 327781, 327782, 327783) c6( ) c7(dP, ae, Y, cBo, cBp, fu, bX, cmk, ad); #c1(SCN1A) c2(NP_001159435) c3(sodium channel protein type 1 subunit alpha isoform 1) c4(64026) c5(64027, 64028, 64029, 64030, 64031, 64032, 64033, 64034, 64035, 64036) c6( ) c7(T, if, oe, ha, EQ, cV, lr, n, iD, ur, ci, akg, C, byV, FR, uu, qN, q, aR, cBw, il, Sw, cBt, hb, nX, HX, lm, hg, Tj, ckC, cC, cB, yr, XK, VX, cBu, ff, r, cm, agF, la, en, g, MV, t, kC, cBr, xw, akq, cBq, bN, bFf, bdW, wQ, Cq, pa, yX, cBs, akm, HN, cBv, avl, m, xg, gD); #c1(SCN1A) c2(NP_001159436) c3(sodium channel protein type I subunit alpha isoform 3) c4(64037) c5(64038, 64039, 64040, 64041, 64042, 64043) c6( ) c7(T, if, oe, ha, EQ, cV, lr, n, iD, ur, ci, akg, C, byV, FR, uu, qN, q, aR, cBw, il, Sw, cBt, hb, nX, HX, lm, hg, Tj, ckC, cC, cB, yr, XK, VX, cBu, ff, r, cm, agF, la, en, g, MV, t, kC, cBr, xw, akq, cBq, bN, bFf, bdW, wQ, Cq, pa, yX, cBs, akm, HN, cBv, avl, m, xg, gD); #c1(SCN1A) c2(NP_008851) c3(sodium channel protein type 1 subunit alpha isoform 2) c4(169117) c5(169118, 169119, 169120, 169121, 169122, 169123) c6( ) c7(T, if, oe, ha, EQ, cV, lr, n, iD, ur, ci, akg, C, byV, FR, uu, qN, q, aR, cBw, il, Sw, cBt, hb, nX, HX, lm, hg, Tj, ckC, cC, cB, yr, XK, VX, cBu, ff, r, cm, agF, la, en, g, MV, t, kC, cBr, xw, akq, cBq, bN, bFf, bdW, wQ, Cq, pa, yX, cBs, akm, HN, cBv, avl, m, xg, gD); #c1(SCN1B) c2(NP_001028) c3(sodium channel subunit beta-1 isoform a precursor) c4(30717) c5( ) c6( ) c7(bL, il, akq, yr, aW, wL, ha, HN, aT, ajM, cBy, hg, EZ, byV, cBz, cBx); #c1(SCN1B) c2(NP_950238) c3(sodium channel subunit beta-1 isoform b precursor) c4(260906) c5(260907, 260908, 260909, 260910, 260911, 260912) c6( ) c7(bL, il, akq, yr, aW, wL, ha, HN, aT, ajM, cBy, hg, EZ, byV, cBz, cBx); #c1(SCN2A) c2(XP_005246807) c3(sodium channel protein type 2 subunit alpha isoform X1) c4(267437, 267438) c5(267439, 267440, 267441, 267442, 267443, 267444, 267445, 267446, 267447, 267448, 267449, 267450, 267451, 267452, 267453, 267454) c6( ) c7(cBA, if, ha, lr, rf, j, nA, bX, byV, FR, qN, il, cBC, cBB, cBD, lm, hg, an, akr, yr, cBE, akq, bN, Cq, avl, Wl); #c1(SCN2A) c2(XP_005246810) c3(sodium channel protein type 2 subunit alpha isoform X2) c4(267455) c5(267456, 267457, 267458, 267459, 267460, 267461, 267462, 267463, 267464, 267465) c6( ) c7(cBA, if, ha, lr, rf, j, nA, bX, byV, FR, qN, il, cBC, cBB, cBD, lm, hg, an, akr, yr, cBE, akq, bN, Cq, avl, Wl); #c1(SCN2B) c2(NP_004579) c3(sodium channel subunit beta-2 precursor) c4(147859) c5(147860, 147861, 147862, 147863, 147864, 147865) c6( ) c7(cBF, wL, ha, avl, ajM, zo); #c1(SCN3A) c2(NP_001075145) c3(sodium channel protein type 3 subunit alpha isoform 2) c4(37246) c5(37247, 37248, 37249, 37250, 37251, 37252) c6( ) c7(acf, ha, lr, cBG, nA, asT, bN); #c1(SCN3A) c2(NP_001075146) c3(sodium channel protein type 3 subunit alpha isoform 3) c4(37253) c5(37254, 37255, 37256, 37257, 37258, 37259) c6( ) c7(acf, ha, lr, cBG, nA, asT, bN); #c1(SCN3A) c2(NP_008853) c3(sodium channel protein type 3 subunit alpha isoform 1) c4(169124) c5(169125, 169126, 169127, 169128, 169129, 169130) c6( ) c7(acf, ha, lr, cBG, nA, asT, bN); #c1(SCN3A) c2(XP_011509913) c3(sodium channel protein type 3 subunit alpha isoform X2) c4(300483) c5(300484, 300485, 300486, 300487, 300488, 300489) c6( ) c7(acf, ha, lr, cBG, nA, asT, bN); #c1(SCN3A) c2(XP_011509915) c3(sodium channel protein type 3 subunit alpha isoform X4) c4(300490) c5(300491, 300492, 300493, 300494, 300495, 300496) c6( ) c7(acf, ha, lr, cBG, nA, asT, bN); #c1(SCN3A) c2(XP_011509916) c3(sodium channel protein type 3 subunit alpha isoform X5) c4(300497) c5(300498, 300499, 300500, 300501, 300502, 300503) c6( ) c7(acf, ha, lr, cBG, nA, asT, bN); #c1(SCN3B) c2(XP_011541199) c3(sodium channel subunit beta-3 isoform X1) c4(339813, 339814) c5(339815, 339816, 339817, 339818, 339819, 339820, 339821, 339822, 339823, 339824, 339825, 339826, 339827, 339828, 339829, 339830, 339831, 339832, 339833, 339834) c6( ) c7(arD, wL, ajM, cBH, tO); #c1(SCN4A) c2(NP_000325) c3(sodium channel protein type 4 subunit alpha) c4(7138) c5(7139, 7140, 7141, 7142, 7143, 7144) c6( ) c7(T, mr, if, cV, blp, aPl, bX, qN, no, aEX, lg, bud, ayH, oj, Ak, BU, HY, lW, aUx, cBl, bw, aLY, HT, dR, lJ, xA, W, ayl, bNi, aOB, cBK, bmE, bt, cBJ); #c1(SCN4B) c2(NP_001135820) c3(sodium channel subunit beta-4 isoform 2) c4(53341) c5(53342, 53343, 53344, 53345, 53346, 53347, 53348) c6( ) c7(cBL, wL, HO, mu); #c1(SCN4B) c2(NP_777594) c3(sodium channel subunit beta-4 isoform 1 precursor) c4(251284) c5(251285, 251286, 251287, 251288, 251289, 251290, 251291, 251292) c6( ) c7(cBL, wL, HO, mu); #c1(SCN5A) c2(NP_000326) c3(sodium channel protein type 5 subunit alpha isoform b) c4(7145) c5(7146, 7147, 7148, 7149, 7150, 7151, 7152, 7153, 7154, 7155) c6( ) c7(bL, cBS, cZ, bHl, T, cBR, mr, cBP, wL, e, aw, EQ, cV, arD, n, bMY, bB, j, ew, fd, atN, ajM, Mk, cBQ, tO, cCa, cBT, cBY, cd, cc, cBZ, lg, sN, vJ, gq, aT, cy, cBO, adm, K, dd, bHm, HO, hm, mu, ajV, eu, A, anR, cBW, aW, bdv, cBV, cBM, cBN, dg, EZ, Jk, Z, bUw, cM, vo, bfW, dr, cBU, cBX, wK, bE, bNi, bVf, Ji, mx, gZ, bUH, QF, bmE, bii, ra, cBz, rf); #c1(SCN5A) c2(NP_001092874) c3(sodium channel protein type 5 subunit alpha isoform c) c4(39675) c5(39676, 39677, 39678, 39679, 39680, 39681) c6( ) c7(bL, cBS, cZ, bHl, T, cBR, mr, cBP, wL, e, aw, EQ, cV, arD, n, bMY, bB, j, ew, fd, atN, ajM, Mk, cBQ, tO, cCa, cBT, cBY, cd, cc, cBZ, lg, sN, vJ, gq, al, cy, cBO, adm, K, dd, bHm, HO, hm, mu, ajV, eu, A, anR, cBW, aW, bdv, cBV, cBM, cBN, dg, EZ, Jk, Z, bUw, cM, vo, bfW, dr, cBU, cBX, wK, bE, bNi, bVf, Ji, mx, gZ, bUH, QF, bmE, bii, ra, cBz, rf); #c1(SCN5A) c2(NP_001092875) c3(sodium channel protein type 5 subunit alpha isoform d) c4(39682) c5(39683, 39684, 39685, 39686, 39687, 39688, 39689, 39690, 39691, 39692) c6( ) c7(bL, cBS, cZ, bHl, T, cBR, mr, cBP, wL, e, aw, EQ, cV, arD, n, bMY, bB, j, ew, fd, atN, ajM, Mk, cBQ, tO, cCa, cBT, cBY, cd, cc, cBZ, lg, sN, vJ, gq, aT, cy, cBO, adm, K, dd, bHm, HO, hm, mu, ajV, eu, A, anR, cBW, aW, bdv, cBV, cBM, cBN, dg, EZ, Jk, Z, bUw, cM, vo, bfW, dr, cBU, cBX, wK, bE, bNi, bVf, Ji, mx, gZ, bUH, QF, bmE, bii, ra, cBz, rf); #c1(SCN5A) c2(NP_001153632) c3(sodium channel protein type 5 subunit alpha isoform e) c4(60792) c5(60793, 60794, 60795, 60796, 60797, 60798, 60799) c6( ) c7(bL, cBS, cZ, bHl, T, cBR, mr, cBP, wL, e, aw, EQ, cV, arD, n, bMY, bB, j, ew, fd, atN, ajM, Mk, cBQ, to, cCa, cBT, cBY, cd, cc, cBZ, lg, sN, vJ, gq, aT, cy, cBO, adm, K, dd, bHm, HO, hm, mu, ajV, eu, A, anR, cBW, aW, bdv, cBV, cBM, cBN, dg, EZ, Jk, Z, bUw, cM, vo, bfW, dr, cBU, cBX, wK, bE, bNi, bVf, Ji, mx, gZ, bUH, QF, bmE, bii, ra, cBz, rf); #c1(SCN5A) c2(NP_001153633) c3(sodium channel protein type 5 subunit alpha isoform f) c4(60800, 60801, 60802) c5(60803, 60804, 60805, 60806, 60807, 60808, 60809, 60810, 60811, 60812, 60813, 60814, 60815, 60816, 60817, 60818, 60819, 60820, 60821, 60822, 60823, 60824) c6( ) c7(bL, cBS, cZ, bHl, T, cBR, mr, cBP, wL, e, aw, EQ, cV, arD, n, bMY, bB, j, ew, fd, atN, ajM, Mk, cBQ, tO, cCa, cBT, cBY, cd, cc, cBZ, lg, sN, vJ, gq, aT, cy, cBO, adm, K, dd, bHm, HO, hm, mu, ajV, eu, A, anR, cBW, aW, bdv, cBV, cBM, cBN, dg, EZ, Jk, Z, bUw, cM, vo, bfW, dr, cBU, cBX, wK, bE, bNi, bVf, Ji, mx, gZ, bUH, QF, bmE, bii, ra, cBz, rf); #c1(SCN5A) c2(XP_006713345) c3(sodium channel protein type 5 subunit alpha isoform X1) c4(287147) c5(287148, 287149, 287150, 287151, 287152, 287153, 287154) c6( ) c7(bL, cBS, cZ, bHl, T, cBR, mr, cBP, wL, e, aw, EQ, cV, arD, n, bMY, bB, j, ew, fd, atN, ajM, Mk, cBQ, tO, cCa, cBT, cBY, cd, cc, cBZ, lg, sN, vJ, gq, aT, cy, cBO, adm, K, dd, bHm, HO, hm, mu, ajV, eu, A, anR, cBW, aW, bdv, cBV, cBM, cBN, dg, EZ, Jk, Z, bUw, cM, vo, bfW, dr, cBU, cBX, wK, bE, bNi, bVf, Ji, mx, gZ, bUH, QF, bmE, bii, ra, cBz, rf); #c1(SCN5A) c2(XP_011532294) c3(sodium channel protein type 5 subunit alpha isoform X3) c4(328433) c5(328434, 328435, 328436, 328437, 328438, 328439) c6( ) c7(bL, cBS, cZ, bHl, T, cBR, mr, cBP, wL, e, aw, EQ, cV, arD, n, bMY, bB, j, ew, fd, atN, ajM, Mk, cBQ, tO, cCa, cBT, cBY, cd, cc, cBZ, lg, sN, vJ, gq, aT, cy, cBO, adm, K, dd, bHm, HO, hm, mu, ajV, eu, A, anR, cBW, aW, bdv, cBV, cBM, cBN, dg, EZ, Jk, Z, bUw, cM, vo, bfW, dr, cBU, cBX, wk, bE, bNi, bVf, Ji, mx, gZ, bUH, OF, bmE, bii, ra, cBz, rf); #c(SCN7A) c2(NP_002967) c3(sodium channel protein type 7 subunit alpha) c4(134051, 134052, 134053) c5(134054, 134055, 134056, 134057, 134058, 134059, 134060, 134061, 134062, 134063, 134064, 134065, 134066, 134067, 134068, 134069, 134070, 134071, 134072, 134073, 134074, 134075, 134076) c6( ) c7(jf, iF, lE, c, tb, Yc, ha, awd, KW, cz, aAh, aur, jN, ww, ajM, bw, il, cm, dx, bN); #c1(SCN8A) c2(NP_001171455) c3(sodium channel protein type 8 subunit alpha isoform 2) c4(70133, 70134) c5(70135, 70136, 70137, 70138, 70139, 70140, 70141, 70142, 70143, 70144, 70145, 70146, 70147, 70148, 70149, 70150, 70151, 70152) c6( ) c7(if, ha, bS, nA, qN, V, cd, aR, eA, Gt, ec, ry, aU, h, aPe, lm, bK, an, he, cB, jf, yr, acR, ih, dq, xw, lv, cCb, vk, BU, qe, ky, cCc, xD, ln, U, uJ, AC, ER, R, zB, xg); #c1(SCN8A) c2(NP_055006) c3(sodium channel protein type 8 subunit alpha isoform 1) c4(178312, 178313) c5(178314, 178315, 178316, 178317, 178318, 178319) c6( ) c7(if, ha, bS, nA, qN, V, cd, aR, eA, Gt, ec, ry, aU, h, aPe, lm, bK, an, he, cB, jf, yr, acR, ih, dq, xw, lv, cCb, vk, BU, qe, ky, cCc, xD, ln, U, uJ, AC, ER, R, zB, xg); #c1(SCN8A) c2(XP_011536953) c3(sodium channel protein type 8 subunit alpha isoform X1) c4(334634) c5(334635, 334636, 334637, 334638, 334639, 334640) c6( ) c7(if, ha, bS, nA, qN, V, cd, aR, eA, Gt, ec, ry, aU, h, aPe, lm, bk, an, he, cB, jf, yr, acR, ih, dq, xw, lv, cCb, vK, BU, qe, ky, cCc, xD, ln, U, uJ, AC, ER, R, zB, xg); #c1(SCN9A) c2(NP_002968) c3(sodium channel protein type 9 subunit alpha) c4(134077) c5( ) c6( ) c7(cZ, aFa, vE, cCf, ha, cCd, cV, bAp, cCe, fu, Ge, ad, aTT, aT, HX, dd, asT, an, ae, aZQ, na, aW, bNA, cCg, aPm, gu, vo, cmk, dr, ccT, eM, xD, bNi, xZ, aqH, uJ, bNy); #c1(SCN5A) c2(XP_011509918) c3(sodium channel protein type 9 subunit alpha isoform X1) c4(300504, 300505) c5(300506, 300507, 300508, 300509, 300510, 300511, 300512, 300513, 300514, 300515, 300516, 300517) c6( ) c7(cZ, aFa, vE, cCf, ha, cCd, cV, bAp, cCe, fu, Ge, ad, aTT, aT, HX, dd, asT, an, ae, aZQ, na, aW, bNA, cCg, aPm, gu, vo, cmk, dr, ccT, eM, xD, bNi, xZ, aqH, uJ, bNy); #c1(SCN9A) c2(XP_011509919) c3(sodium channel protein type 9 subunit alpha isoform X2) c4(300518, 300519) c5(300520, 300521, 300522, 300523, 300524, 300525, 300526, 300527, 300528, 300529, 300530, 300531) c6( ) c7(cZ, aFa, vE, cCf, ha, cCd, cV, bAp, cCe, fu, Ge, ad, aTT, aT, HX, dd, asT, an, ae, aZQ, na, aW, bNA, cCg, aPm, gu, vo, cmk, dr, ccT, eM, xD, bNi, xZ, aqH, uJ, bNy); #c1(SCN9A) c2(XP_011509920) c3(sodium channel protein type 9 subunit alpha isoform X3) c4( ) c5(300532, 300533, 300534, 300535, 300536, 300537) c6( ) c7(cZ, aFa, vE, cCf, ha, cCd, cV, bAp, cCe, fu, Ge, ad, aTT, aT, HX, dd, asT, an, ae, aZQ, na, aW, bNA, cCg, aPm, gu, vo, cmk, dr, ccT, eM, xD, bNi, xZ, aqH, uJ, bNy); #c1(SCNM1) c2(NP_001191785) c3(sodium channel modifier 1 isoform 2) c4( ) c5(81758, 81759, 81760, 81761, 81762) c6( ) c7(aU, aR); #c1(SCNM1) c2(NP_076946) c3(sodium channel modifier 1 isoform 1) c4(214336, 214337) c5(214338, 214339, 214340, 214341, 214342, 214343, 214344) c6( ) c7(aU, aR); #c1(SCNN1A) c2(NP_001029) c3(amiloride-sensitive sodium channel subunit alpha isoform 1) c4( ) c5( ) c6( ) c7(bL, bM, T, dD, ahS, fR, sQ, oe, cz, oR, MF, ah, eq, aT, lQ, an, wt, aa, kl, mR, cCh, vj, cm, uC, cM, qa, kT, c, uR, uo, ut, Lb, dy, ei, bD, ao, clQ); #c1(SCNN1A) c2(NP_001153047) c3(amiloride-sensitive sodium channel subunit alpha isoform 3) c4(60361, 60362) c5(60363, 60364, 60365, 60366, 60367, 60368) c6( ) c7(bL, bM, T, dD, ahS, fR, s, oe, cz, OR, MF, ah, eq, aT, lQ, an, wt, aa, kl, mR, cCh, vj, cm, uC, cM, qa, kT, c, uR, uo, ut, Lb, dy, ei, bD, ao, clQ); #c1(SCNN1A) c2(NP_001153048) c3(amiloride-sensitive sodium channel subunit alpha isoform 2) c4(60369) c5(60370, 60371, 60372, 60373, 60374, 60375, 60376, 60377, 60378, 60379, 60380, 60381, 60382, 60383, 60384, 60385, 60386, 60387) c6( ) c7(bL, bM, T, dD, ahS, fR, sQ, oe, cz, oR, MF, ah, eq, aT, lQ, an, wt, aa, kl, mR, cCh, vj, cm, uC, cM, qa, kT, c, uR, uo, ut, Lb, dy, ei, bD, ao, clQ); #c1(SCNN1B) c2(NP_000327) c3(amiloride-sensitive sodium channel subunit beta) c4(7156) c5( ) c6( ) c7(bL, aLZ, dD, ahS, iV, vj, oe, cz, afq, MF, ah, eq, fr, clQ, uC, vP, ao, aa); #c1(SCNN1B) c2(XP_011544215) c3 (amiloride-sensitive sodium channel subunit beta isoform X1) c4(343350) c5(343351, 343352, 343353, 343354, 343355, 343356, 343357) c6( ) c7(bL, aLZ, dD, ahS, iV, vj, oe, cz, afq, MF, ah, eq, fr, clQ, uC, vP, ao, aa); #c1(SCNN1B) c2(XP_011544216) c3(amiloride-sensitive sodium channel subunit beta isoform X2) c4( ) c5(343358, 343359, 343360, 343361, 343362) c6( ) c7(bL, aLZ, dD, ahS, iV, vj, oe, cz, afq, MF, ah, eq, fr, clQ, uC, vP, ao, aa); #c1(SCNN1D) c2(XP_011540201) c3(amiloride-sensitive sodium channel subunit delta isoform X1) c4( ) c5(338604, 338605) c6( ) c7(Y, cE, eA); #c1(SCNN1D) c2(XP_011540207) c3(amiloride-sensitive sodium channel subunit delta isoform X7) c4( ) c5(338606) c6( ) c7(Y, cE, eA); #c1(SCNN1D) c2(XP_011540212) c3(amiloride-sensitive sodium channel subunit delta isoform X12) c4( ) c5(338607) c6( ) c7(Y, cE, eA); #c1(SCNN1D) c2(XP_011540215) c3(amiloride-sensitive sodium channel subunit delta isoform X15) c4( ) c5(338608) c6( ) c7(Y, cE, eA); #c1(SCNN1D) c2(XP_011540219) c3(amiloride-sensitive sodium channel subunit delta isoform X19) c4( ) c5(338609) c6( ) c7(Y, cE, eA); #c1(SCNN1D) c2(XP_011540224) c3(amiloride-sensitive sodium channel subunit delta isoform X24) c4(338610) c5( ) c6( ) c7(Y, cE, eA); #c1(SCNN1D) c2(XP_011540228) c3(amiloride-sensitive sodium channel subunit delta isoform X27) c4(338611) c5(338612, 338613, 338614, 338615, 338616) c6( ) c7(Y, cE, eA); #c1(SCNN1D) c2(XP_011540230) c3(amiloride-sensitive sodium channel subunit delta isoform X29) c4( ) c5(338617) c6( ) c7(Y, cE, eA); #c1(SCNN1D) c2(XP_011540233) c3(amiloride-sensitive sodium channel subunit delta isoform X32) c4(338618, 338619) c5(338620, 338621) c6( ) c7(Y, cE, eA); #c1(SCNN1D) c2(XP_011540235) c3(amiloride-sensitive sodium channel subunit delta isoform X33) c4( ) c5(338622, 338623, 338624, 338625, 338626) c6( ) c7(Y, cE, eA); #c1(SCNN1D) c2(XP_011540236) c3(amiloride-sensitive sodium channel subunit delta isoform X34) c4( ) c5(338627, 338628, 338629, 338630, 338631, 338632, 338633, 338634, 338635, 338636, 338637, 338638, 338639, 338640) c6( ) c7(Y, cE, eA); #c1(SCNN1D) c2(NP_001030) c3(amiloride-sensitive sodium channel subunit gamma) c4(31077) c5( ) c6( ) c7(cCi, dD, T, aE, ahS, iV, oe, ff, cz, ao, afq, MF, eq, en, clQ, azh, aa); #c1(SC01) c2(NP_004580) c3(protein SC01 homolog, mitochondrial) c4(147866, 147867) c5(147868, 147869, 147870, 147871, 147872, 147873, 147874, 147875, 147876, 147877, 147878, 147879) c6( ) c7(cCj, cCl, hb, hl, dV, vc, acJ, aln, kC, cCk); #c1(SC02) c2(NP_001162582) c3(protein SC02 homolog, mitochondrial precursor) c4(66376, 66377) c5(66378, 66379, 66380, 66381, 66382, 66383, 66384, 66385, 66386, 66387, 66388, 66389, 66390, 66391, 66392, 66393, 66394, 66395, 66396, 66397, 66398, 66399, 66400, 66401, 66402, 66403, 66404, 66405) c6( ) c7(fu, arX, xt, cc, hb, ds, kf, bdh, vc, bl, ajU, cCm, apl, cCn, aq, px, kC, apq, SE, Be, acJ, B); #c1(SCP2D1) c2(NP_848578) c3(SCP2 sterol-binding domain-containing protein 1) c4(253878) c5(253879, 253880, 253881, 253882, 253883, 253884) c6( ) c7(cm, cd); #c1(SCPEP1) c2(NP_067639) c3(retinoid-inducible serine carboxypeptidase precursor) c4(209141) c5(209142, 209143, 209144, 209145, 209146, 209147, 209148, 209149) c6(209150, 209151) c7(T, iT, iU, WB, aW, cz, aT, fd, bu); #c1(SCRIB) c2(NP_056171) c3 (protein scribble homolog isoform b) c4(186102, 186103, 186104) c5(186105, 186106, 186107, 186108, 186109, 186110, 186111, 186112, 186113, 186114, 186115, 186116, 186117, 186118, 186119, 186120, 186121, 186122) c6( ) c7(bF, T, lg, lp, aW, aT, By, X); #c1(SCRIB) c2(NP_874365) c3(protein scribble homolog isoform a) c4(256622) c5(256623, 256624, 256625, 256626, 256627, 256628) c6( ) c7(bF, T, lg, lp, aW, aT, By, X); #c1(SCRN1) c2(NP_001138986) c3(secernin-1 isoform b) c4(58464, 58465, 58466) c5(58467, 58468, 58469, 58470, 58471, 58472, 58473, 58474, 58475, 58476) c6( ) c7(T, bE, aA, bB, r, aF, K, be, q); #c1(SCRN1) c2(NP_001138987) c3(secernin-1 isoform c) c4(58477, 58478) c5(58479, 58480, 58481, 58482, 58483, 58484, 58485, 58486, 58487, 58488, 58489, 58490, 58491, 58492, 58493, 58494, 58495) c6( ) c7(T, bE, aA, bB, r, aF, K, be, q); #c1(SCRN1) c2(NP_055581) c3(secernin-1 isoform a) c4(182076, 182077) c5(182078, 182079, 182080, 182081, 182082, 182083, 182084, 182085, 182086, 182087, 182088, 182089, 182090, 182091, 182092, 182093, 182094, 182095, 182096, 182097, 182098, 182099, 182100) c6( ) c7(T, bE, aA, bB, r, aF, K, be, q); #c1(SCT) c2(XP_011518583) c3(secretin isoform X1) c4(312740) c5(312741, 312742, 312743, 312744, 312745, 312746) c6( ) c7(kV, oO, T, os, aC, ow, qb, ex, jh, rf, bvq, oZ, ci, aY, hF, eW, br, x, azY, jz, aE, u, arV, aMj, eV, adV, he, iL, kl, je, iU, qt, Y, aNi, aq, lo, aB, g, aot, iR, LV, cm, bz, bw, bN, OC, ck, CC, BU, iM, iT, ag, AR, wB, aiM, fP, cj, U, pg, k, bt, B, aya); #c1(SCTR) c2(NP_002971) c3(secretin receptor precursor) c4(134078) c5(134079, 134080, 134081, 134082, 134083, 134084, 134085, 134086) c6( ) c7(nZ, T, aC, jz, eJ, s, Do, aE, lt, AR, aq, ci, g, iR, ae, B, ad); #c1(SCUBE1) c2(NP_766638) c3(signal peptide, CUB and EGF-like domain-containing protein 1 precursor) c4(249467, 249468) c5(249469, 249470, 249471, 249472, 249473, 249474, 249475, 249476, 249477, 249478, 249479, 249480) c6( ) c7(dA); #c1(SCUBE1) c2(XP_005261809) c3(signal peptide, CUB and EGF-like domain-containing protein 1 isoform X1) c4(277627, 277628) c5(277629, 277630, 277631, 277632, 277633, 277634, 277635) c6( ) c7(dA); #c1(SCUBE1) c2(XP_011528693) c3(signal peptide, CUB and EGF-like domain-containing protein 1 isoform X2) c4(323950, 323951) c5(323952, 323953, 323954, 323955, 323956, 323957, 323958, 323959, 323960, 323961, 323962, 323963) c6( ) c7(dA); #c1(SCUBE2) c2(NP_001164161) c3(signal peptide, CUB and EGF-like domain-containing protein 2 isoform 2 precursor) c4(66650) c5(66651, 66652, 66653, 66654, 66655, 66656) c6( ) c7(T, aW, aT, at, n, dL); #c1(SCUBE2) c2(NP_066025) c3(signal peptide, CUB and EGF-like domain-containing protein 2 isoform 1 precursor) c4(206796) c5(206797, 206798, 206799, 206800, 206801, 206802) c6( ) c7(T, aW, aT, at, n, dL); #c1(SCUBE2) c2(XP_005253089) c3(signal peptide, CUB and EGF-like domain-containing protein 2 isoform X1) c4(272139) c5(272140, 272141, 272142, 272143, 272144, 272145) c6( ) c7(T, aW, aT, at, n, dL); #c1(SCUBE2) c2(XP_005253090) c3(signal peptide, CUB and EGF-like domain-containing protein 2 isoform X3) c4(272146) c5(272147, 272148, 272149, 272150, 272151, 272152) c6( ) c7(T, aW, al, at, n, dL); #c1(SCUBE3) c2(NP_689966) c3(signal peptide, CUB and EGF-like domain-containing protein 3 isoform 1 precursor) c4(244609) c5(244610, 244611, 244612, 244613, 244614, 244615, 244616, 244617) c6(244618, 244619) c7(T, fr); #c1(SCUBE3) c2(XP_005249000) c3(signal peptide, CUB and EGF-like domain-containing protein 3 isoform X1) c4(268839) c5(268840, 268841, 268842, 268843, 268844, 268845, 268846, 268847) c6(268848, 268849) c7(T, fr); #c1(SCYL1) c2(NP_001041683) c3(N-terminal kinase-like protein isoform B) c4(34290, 34291, 34292) c5(34293, 34294, 34295, 34296, 34297, 34298, 34299, 34300, 34301, 34302, 34303, 34304, 34305, 34306, 34307, 34308) c6( ) c7(T, iW, jh, aA, zh, ci, ze, qN, fr, alq, zo, ad, eA, x, ft, ib, aE, dV, h, hn, zg, ae, d, zb, ff, aF, en, iV, t, lk, bz, ve, iK, ck, bP, zr, iT, aps, U, zl, k, eg, zk, bt); #c1(SCYL1) c2(NP_065731) c3(N-terminal kinase-like protein isoform A) c4(205115, 205116) c5(205117, 205118, 205119, 205120, 205121, 205122) c6( ) c7(T, iW, jh, aA, zh, ci, ze, qN, fr, alq, zo, ad, eA, x, ft, ib, aE, dV, h, hn, zg, ae, d, zb, ff, aF, en, iV, t, lk, bz, ve, ik, ck, bP, zr, iT, aps, U, zl, k, eg, zk, bt); #c1(SCYL1) c2(XP_005274177) c3(N-terminal kinase-like protein isoform X2) c4(284969) c5(284970, 284971, 284972, 284973, 284974, 284975, 284976, 284977, 284978, 284979, 284980, 284981) c6( ) c7(T, iW, jh, aA, zh, ci, ze, qN, fr, alq, zo, ad, eA, x, ft, ib, aE, dV, h, hn, zg, ae, d, zb, ff, aF, en, iV, t, lk, bz, ve, ik, ck, bP, zr, iT, aps, U, zl, k, eg, zk, bt); #c1(SCYL1) c2(XP_005274178) c3(N-terminal kinase-like protein isoform X3) c4(284982) c5(284983, 284984, 284985, 284986, 284987, 284988, 284989, 284990, 284991, 284992, 284993, 284994) c6( ) c7(T, iW, jh, aA, zh, ci, ze, qN, fr, alq, zo, ad, eA, x, ft, ib, aE, dV, h, hn, zg, ae, d, zb, ff, aF, en, iV, t, lk, bz, ve, ik, ck, bP, zr, iT, aps, U, zl, k, eg, zk, bt); #c1(SCYL3) c2(NP_065156) c3(protein-associating with the carboxyl-terminal domain of ezrin isoform 1) c4(204327, 204328) c5(204329, 204330, 204331, 204332, 204333, 204334, 204335, 204336, 204337, 204338, 204339, 204340) c6( ) c7(bA); #c1(SCYL3) c2(NP_851607) c3(protein-associating with the carboxyl-terminal domain of ezrin isoform 2) c4(254582) c5(254583, 254584, 254585, 254586, 254587, 254588, 254589, 254590, 254591, 254592) c6( ) c7(bA); #c1(SDC1) c2(NP_001006947) c3(syndecan-1 precursor) c4(22380) c5( ) c6( ) c7(T, aC, aiH, if, fd, ci, apQ, aE, K, wt, dQ, eL, li, aW, g, nr, eF, cm, eM, nB, cj, m, R, aw, n, qN, aOJ, Bl, ft, zw, pe, eV, By, dL, fe, aq, CQ, Zj, Wt, hk, hL, eo, abW, B, fM, fG, yk, eX, aA, v, V, q, eA, XX, blr, lg, qK, aT, ae, dl, jf, oV, Y, r, en, apx, aom, t, cl, ck, kP, of, gX, bt, hS, Mb, id, jh, ad, dP, av, fX, ib, Dq, ji, MG, qZ, ff, eH, cr, eZ, iV, bz, LB, y); #c1(SDC1) c2(XP_005262679) c3(syndecan-1 isoform X3) c4( ) c5(278038, 278039, 278040, 278041, 278042, 278043, 278044, 278045, 278046, 278047) c6( ) c7(T, aC, aiH, if, fd, ci, apQ, aE, K, wt, dQ, eL, li, aW, g, nr, eF, cm, eM, nB, cj, m, R, aw, n, qN, aOJ, Bl, ft, zw, pe, eV, By, dL, fe, aq, CQ, Zj, Wt, hk, hl, eo, abW, B, fM, fG, yk, eX, aA, v, V, q, eA, XX, blr, lg, qK, aT, ae, dl, jf, oV, Y, r, en, apx, aom, t, cl, ck, kP, of, gX, bt, hS, Mb, id, jh, ad, dP, av, fx, ib, Dq, ji, MG, qZ, ff, eH, cr, eZ, iV, bz, LB, y); #c1(SDC2) c2(NP_002989) c3(syndecan-2 precursor) c4(134206, 134207, 134208) c5(134209, 134210, 134211, 134212, 134213, 134214, 134215, 134216, 134217, 134218, 134219, 134220, 134221, 134222, 134223, 134224, 134225, 134226, 134227, 134228, 134229, 134230) c6( ) c7(bL, T, kh, dD, AS, fG, bB, oY, v, eq, ci, XM, cd, q, ai, aao, eJ, Na, aE, Db, qK, yg, aT, h, Nc, K, uD, dQ, DA, eG, TY, aW, Y, ev, eH, r, eF, fq, pb, LB, vW, Dy, ac, nB, bE, w, BL, cj, dy, y, B); #c1(SDC2) c2(XP_005251076) c3(syndecan-2 isoform X3) c4(270747) c5(270748, 270749) c6( ) c7(bL, T, kh, dD, AS, fG, bB, oY, v, eq, ci, XM, cd, q, ai, aao, eJ, Na, aE, Db, qK, yg, aT, h, Nc, K, uD, dQ, DA, eG, TY, aW, Y, ev, eH, r, eF, fq, pb, LB, vW, Dy, ac, nB, bE, w, BL, cj, dy, y, B); #c1(SDC2) c2(XP_011515514) c3(syndecan-2 isoform X4) c4( ) c5(308359, 308360, 308361, 308362, 308363, 308364, 308365, 308366) c6( ) c7(bL, T, kh, dD, AS, fG, bB, oY, v, eq, ci, XM, cd, q, ai, aao, eJ, Na, aE, Db, qK, yg, aT, h, Nc, K, uD, dQ, DA, eG, TY, aW, Y, ev, eH, r, eF, fq, pb, LB, vW, Dy, ac, nB, bE, w, BL, cj, dy, y, B); #c1(SDC3) c2(XP_011540764) c3(syndecan-3 isoform X1) c4(339340) c5( ) c6( ) c7(aW, Pl, uY, v, uV, y, eA); #c1(SDC3) c2(XP_011540768) c3(syndecan-3 isoform X5) c4( ) c5(339341, 339342, 339343, 339344, 339345, 339346, 339347, 339348, 339349, 339350, 339351, 339352) c6( ) c7(aW, Pl, uY, v, uV, y, eA); #c1(SDC4) c2(NP_002990) c3(syndecan-4 precursor) c4(134231, 134232) c5(134233, 134234, 134235, 134236, 134237, 134238, 134239) c6( ) c7(oO, bM, T, dD, aw, bB, eq, ci, cd, K, lQ, aiJ, hm, DA, Y, eH, cr, hf, eF, ell, eM, bE, w, do, bt); #c1(SDC4) c2(XP_011527279) c3(syndecan-4 isoform X1) c4( ) c5(321913, 321914, 321915, 321916, 321917) c6( ) c7(oO, bM, T, dD, aw, bB, eq, ci, cd, K, lQ, aiJ, hm, DA, Y, eH, cr, hf, eF, eU, eM, bE, w, do, bt); #c1(SDCBP) c2(NP_001007069) c3(syntenin-1 isoform 2) c4(22449) c5(22450, 22451, 22452, 22453, 22454) c6( ) c7(T, dA, aW, Y, al, aA, n, aF, bQN, iP, cp); #c1(SDCBP) c2(NP_001007071) c3(syntenin-1 isoform 3) c4(22455) c5(22456, 22457, 22458, 22459, 22460, 22461, 22462, 22463) c6( ) c7(T, dA, aW, Y, aT, aA, n, aF, bQN, iP, cp); #c1(SDCBP) c2(NP_005616) c3(syntenin-1 isoform 1) c4(157964) c5(157965, 157966, 157967, 157968, 157969, 157970, 157971, 157972) c6( ) c7(T, dA, aW, Y, aT, aA, n, aF, bQN, iP, cp); #c1(SDCBP) c2(XP_011515873) c3(syntenin-I isoform X1) c4( ) c5(308852, 308853, 308854, 308855, 308856) c6( ) c7(T, dA, aW, Y, aT, aA, n, aF, bQN, iP, cp); #c1(SDCBP2) c2(NP_001186713) c3(syntenin-2 isoform a) c4(78396, 78397) c5(78398, 78399, 78400, 78401, 78402, 78403, 78404, 78405, 78406, 78407, 78408, 78409, 78410, 78411, 78412) c6( ) c7(bQN); #c1(SDCBP2) c2(NP_056500) c3(syntenin-2 isoform b) c4( ) c5(187793, 187794, 187795, 187796, 187797) c6( ) c7(bQN); #c1(SDCCAG3) c2(NP_001034796) c3(serologically defined colon cancer antigen 3 isoform 1) c4(32307) c5(32308, 32309, 32310, 32311, 32312, 32313) c6( ) c7(bB); #c1(SDCCAG3) c2(NP_001034797) c3(serologically defined colon cancer antigen 3 isoform 3) c4(32314) c5(32315, 32316, 32317, 32318, 32319, 32320) c6( ) c7(bB); #c1(SDCCAG3) c2(NP_006634) c3(serologically defined colon cancer antigen 3 isoform 2) c4(166754) c5(166755, 166756, 166757, 166758, 166759, 166760) c6( ) c7(bB); #c1(SDCCAG8) c2(NP_006633) c3(serologically defined colon cancer antigen 8) c4(166747) c5(166748, 166749, 166750, 166751, 166752, 166753) c6( ) c7(lR, cCo, h, v, Tp, V); #c1(SDCCAG8) c2(XP_006725027) c3(serologically defined colon cancer antigen 8 isoform X3) c4(296543) c5(296544, 296545, 296546, 296547, 296548, 296549) c6( ) c7(lR, cCo, h, v, Tp, V); #c1(SDCCAG8) c2(XP_006725029) c3(serologically defined colon cancer antigen 8 isoform X9) c4(296550) c5(296551, 296552, 296553, 296554, 296555, 296556) c6( ) c7(lR, cCo, h, v, Tp, V); #c1(SDCCAG8) c2(XP_006725036) c3(serologically defined colon cancer antigen 8 isoform X18) c4(296557) c5(296558, 296559, 296560, 296561, 296562, 296563) c6( ) c7(lR, cCo, h, v, Tp, V); #c1(SDCCAG8) c2(XP_011544737) c3 (serologically defined colon cancer antigen 8 isoform X12) c4(343808) c5(343809, 343810, 343811, 343812, 343813, 343814) c6( ) c7(lR, cCo, h, v, Tp, V); #c1(SDF2) c2(NP_008854) c3(stromal cell-derived factor 2 precursor) c4(169131) c5(169132, 169133, 169134, 169135, 169136, 169137) c6( ) c7(bL, aW, bM, aT); #c1(SDFZL1) c2(NP_071327) c3(stromal cell-derived factor 2-like protein 1 precursor) c4(210994) c5(210995, 210996, 210997, 210998, 210999, 211000) c6( ) c7(cd, ad); #c1(SDF4) c2(XP_011539858) c3(45 kDa calcium-binding protein isoform X1) c4(338140) c5(338141, 338142, 338143, 338144, 338145, 338146) c6( ) c7(bM, T, Vo, aA, bB, fr, ci, q, ad, Hx, hd, jz, aE, aT, hD, dL, lQ, ae, eL, aW, Y, ev, lb, cr, r, dN, aF, Xk, dw, eE, bz, ar, Dy, bE, z, U, aps, bt, L); #c1(SDHA) c2(NP_001281261) c3 (succinate dehydrogenase [ubiquinone] flavoprotein subunit, mitochondrial isoform 2) c4(111607) c5(111608, 111609, 111610, 111611, 111612, 111613) c6( ) c7(cCr, yo, fb, cc, hb, if, ajU, anx, mx, VB, cCp, dL, fr, qN, cCq, cb, cm, cCs, eL, ad, cCt); #c1(SDHA) c2(NP_004159) c3(succinate dehydrogenase [ubiquinone] flavoprotein subunit, mitochondrial isoform 1) c4(143954) c5(143955, 143956, 143957, 143958, 143959, 143960) c6( ) c7(cCr, yo, fb, cc, hb, if, ajU, anx, mx, VB, cCp, dL, fr, qN, cCq, cb, cm, cCs, eL, ad, cCt); #c1(SDHAF1) c2(NP_001036096) c3(succinate dehydrogenase assembly factor 1, mitochondrial) c4(33976) c5(33977, 33978, 33979, 33980, 33981, 33982) c6( ) c7(cCp, bFd, cCt); #c1(SDHAF2) c2(NP_060311) c3(succinate dehydrogenase assembly factor 2, mitochondrial) c4(195316) c5(195317, 195318, 195319, 195320, 195321, 195322) c6( ) c7(ae, T, BC, YE, Do, pX, anE, cCu, ci, anx, Hl, bz, VB); #c1(SDHAF4) c2(NP_660310) c3(succinate dehydrogenase assembly factor 4, mitochondrial precursor) c4(240860) c5(240861, 240862, 240863, 240864, 240865, 240866, 240867, 240868) c6(240869, 240870) c7(bL); #c1(SDHC) c2(NP_001030588) c3(succinate dehydrogenase cytochrome b560 subunit, mitochondrial isoform 2 precursor) c4(31034) c5(31035, 31036, 31037, 31038, 31039, 31040) c6( ) c7(T, cz, KO, hy, aNf, fr, cCv, ci, ad, hd, nv, nX, cU, fb, ae, Jg, nO, eH, aq, Hu, eF, es, eM, iA, go, mg, hR, QF, anx, B, VB); #c1(SDHC) c2(NP_001030589) c3(succinate dehydrogenase cytochrome b560 subunit, mitochondrial isoform 3 precursor) c4(31041) c5(31042, 31043, 31044, 31045, 31046, 31047) c6( ) c7(T, cz, KO, hy, aNf, fr, cCv, ci, ad, hd, nv, nX, cU, fb, ae, Jg, nO, eH, aq, Hu, eF, es, eM, iA, go, mg, hR, QF, anx, B, VB); #c1(SDHC) c2(NP_001030590) c3 (succinate dehydrogenase cytochrome b560 subunit, mitochondrial isoform 4 precursor) c4(31048) c5(31049, 31050, 31051, 31052, 31053, 31054) c6( ) c7(T, cz, KO, hy, aNf, fr, cCv, ci, ad, hd, nv, nX, cU, fb, ae, Jg, nO, eH, aq, Hu, eF, es, eM, iA, go, mg, hR, QF, anx, B, VB); #c1(SDHC) c2(NP_002992) c3(succinate dehydrogenase cytochrome b560 subunit, mitochondrial isoform 1 precursor) c4(134240) c5(134241, 134242, 134243, 134244, 134245, 134246) c6( ) c7(T, cz, KO, hy, aNf, fr, cCv, ci, ad, hd, nv, nX, cU, fb, ae, Jg, nO, eH, aq, Hu, eF, es, eM, iA, go, mg, hR, QF, anx, B, VB); #c1(SDHD) c2(NP_002993) c3(succinate dehydrogenase [ubiquinone] cytochrome b small subunit, mitochondrial isoform a precursor) c4(134247) c5(134248, 134249, 134250, 134251, 134252, 134253) c6( ) c7(bLx, atT, T, aAx, aA, n, KO, aNf, oH, ci, hy, cCx, qN, fr, chj, AW, hd, oT, nv, nX, pX, KN, bj, jj, fb, Zm, cCt, bWL, cCw, jr, s, Y, cCp, Hu, cm, dr, aHY, es, Ew, iA, VB, hR, bPs, QF, Gg, anx, kS, abh); #c1(SDK1) c2(NP_689957) c3(protein sidekick-1 isoform 1) c4(244531, 244532) c5(244533, 244534, 244535, 244536, 244537, 244538, 244539, 244540, 244541, 244542, 244543, 244544) c6( ) c7(sk, ac, aw, cz, cm, h, he, ae, cd, ad, hS); #c1(SDK2) c2(NP_001138424) c3(protein sidekick-2 precursor) c4(56660, 56661) c5(56662, 56663, 56664, 56665, 56666, 56667, 56668, 56669, 56670, 56671, 56672, 56673, 56674) c6( ) c7(aj, ww); #c1(SDK2) c2(XP_011523216) c3 (protein sidekick-2 isoform X1) c4(317745) c5(317746, 317747, 317748, 317749, 317750, 317751) c6( ) c7(aj, ww); #c1(SDK2) c2(XP_011523217) c3(protein sidekick-2 isoform X2) c4( ) c5(317752, 317753, 317754, 317755) c6( ) c7(aj, ww); #c1(SDPR) c2(NP_004648) c3(serum deprivation-response protein) c4(148502) c5(148503, 148504, 148505, 148506, 148507, 148508) c6( ) c7(aW, mC, eZ, aT, bas, fL, cd); #c1(SDR42E1) c2(XP_011521772) c3(short-chain dehydrogenase/reductase family 42E member 1 isoform X1) c4(316461) c5(316462, 316463, 316464, 316465, 316466, 316467) c6( ) c7(aii); #c1(SDR9C7) c2(NP_683695) c3(short-chain dehydrogenase/reductase family 9C member 7) c4(242395) c5(242396, 242397, 242398, 242399, 242400, 242401) c6( ) c7(it); #c1(SDS) c2(NP_006834) c3(L-serine dehydratase/L-threonine deaminase) c4(168531, 168532) c5(168533, 168534, 168535, 168536, 168537, 168538, 168539, 168540, 168541, 168542, 168543, 168544) c6( ) c7(bL, T, aZA, aFQ, if, Am, fd, jh, cz, bBX, bB, asA, bS, j, ct, gf, ci, dG, v, qN, lR, fr, ad, wD, mj, b, cc, vJ, vz, ds, i, dv, By, dL, bj, K, H, vx, ae, af, qk, ao, eL, fb, yo, akV, arl, A, Fm, Y, Rj, ff, eZ, dg, hp, en, g, Z, kC, qs, pb, op, es, VJ, Ew, iA, bE, vA, U, cj, vy, XO, anx, hh, y, bt, VB, L); #c1(SEC11A) c2(NP_001258847) c3(signal peptidase complex catalytic subunit SEC11A isoform 5) c4( ) c5(96585, 96586, 96587, 96588, 96589) c6( ) c7(aF, aA); #c1(SEC11A) c2(NP_055115) c3(signal peptidase complex catalytic subunit SEC11A isoform 2) c4(179029, 179030) c5(179031, 179032, 179033, 179034, 179035, 179036, 179037) c6( ) c7(aF, aA); #c1(SEC11C) c2(NP_150596) c3(signal peptidase complex catalytic subunit SEC11C) c4(228124, 228125) c5(228126, 228127, 228128, 228129, 228130, 228131, 228132, 228133, 228134, 228135) c6( ) c7(aC); #c1(SEC13) c2(NP_001129498) c3(protein SEC13 homolog isoform 3) c4(51699, 51700, 51701, 51702) c5(51703, 51704, 51705, 51706, 51707, 51708, 51709) c6( ) c7(VB); #c1(SEC13) c2(NP_001129704) c3(protein SEC13 homolog isoform 2) c4( ) c5(52222, 52223, 52224, 52225, 52226) c6( ) c7(VB); #c1(SEC13) c2(NP_109598) c3(protein SEC13 homolog isoform 4) c4(219768) c5(219769, 219770, 219771, 219772, 219773) c6( ) c7(VB); #c1(SEC13) c2(NP_899195) c3(protein SEC13 homolog isoform 1) c4( ) c5(257994, 257995, 257996, 257997, 257998, 257999) c6( ) c7(VB); #c1(SEC13) c2(XP_005265435) c3 (protein SEC13 homolog isoform X3) c4( ) c5(279550, 279551) c6( ) c7(VB); #c1(SEC13) c2(XP_011532298) c3(protein SEC13 homolog isoform X4) c4( ) c5(328440, 328441, 328442, 328443, 328444) c6( ) c7(VB); #c1(SEC14L1) c2(NP_001137470) c3(SEC14-like protein 1 isoform a) c4(55906) c5(55907, 55908, 55909, 55910, 55911, 55912, 55913, 55914, 55915, 55916, 55917, 55918, 55919, 55920, 55921, 55922, 55923, 55924, 55925, 55926) c6( ) c7(t, nC, ae, ad); #c1(SEC14L1) c2(NP_001137473) c3(SEC14-like protein 1 isoform c) c4(55927) c5( ) c6( ) c7(t, nC, ae, ad); #c1(SEC14L1) c2(NP_001191337) c3(SEC14-like protein 1 isoform b) c4(81420) c5(81421, 81422, 81423, 81424, 81425, 81426, 81427, 81428, 81429, 81430) c6( ) c7(t, nC, ae, ad); #c1(SEC14L2) c2(NP_001191133) c3(SEC14-like protein 2 isoform 3) c4(81066) c5(81067, 81068, 81069, 81070, 81071, 81072) c6( ) c7(oO, bM, T, if, eb, bB, cL, bvx, ci, AW, K, hu, hH, cp, aa, dH, aW, hl, g, eE, eM, Jr, aiM, qR, QO, n, fr, qN, eW, ft, cU, h, dL, A, mR, aq, at, Xa, aAl, aCD, zl, B, Dw, AS, bvt, jx, aA, V, q, o, jF, lg, cei, aT, ae, Y, r, afq, lo, aDm, en, iK, pb, ig, bvy, AH, vi, Ke, aDy, iD, ad, hX, av, F, hD, uQ, atJ, d, KS, ff, cr, eZ, aF, xH, hp, iV, bz, bE, z, aCl, y, WX, hv); #c1(SEC14L2) c2(NP_001278861) c3(SEC14-like protein 2 isoform 4) c4( ) c5(110738, 110739, 110740, 110741, 110742) c6( ) c7(oO, bM, T, if, eb, bB, cL, bvx, ci, AW, K, hu, hH, cp, aa, dH, aW, hl, g, eE, eM, Jr, aiM, qR, QO, n, fr, qN, eW, ft, cU, h, dL, A, mR, aq, at, Xa, aAl, aCD, zl, B, Dw, AS, bvt, jx, aA, V, q, o, jF, lg, cei, aT, ae, Y, r, afq, lo, aDm, en, iK, pb, ig, bvy, AH, vi, Ke, aDy, iD, ad, hX, av, F, hD, uQ, atJ, d, KS, ff, cr, eZ, aF, xH, hp, iV, bz, bE, z, aCl, y, WX, hv); #c1(SEC14L2) c2(NP_036561) c3(SEC14-like protein 2 isoform 1) c4(174409, 174410) c5(174411, 174412, 174413, 174414, 174415, 174416, 174417) c6( ) c7(oO, bM, T, if, eb, bB, cL, bvx, ci, AW, K, hu, hH, cp, aa, dH, aW, hl, g, eE, eM, Jr, aiM, qR, oO, n, fr, qN, eW, ft, cU, h, dL, A, mR, aq, at, Xa, aAl, aCD, zl, B, Dw, AS, bvt, jx, aA, V, q, o, jF, lg, cei, aT, ae, Y, r, afq, lo, aDm, en, iK, pb, ig, bvy, AH, vi, Ke, aDy, iD, ad, hX, av, F, hD, uQ, atJ, d, KS, ff, cr, eZ, aF, xH, hp, iV, bz, bE, z, aCl, y, WX, hv); #c1(SEC14L2) c2(NP_203740) c3(SEC14-like protein 2 isoform 2) c4(228468) c5(228469, 228470, 228471, 228472, 228473, 228474) c6( ) c7(oO, bM, T, if, eb, bB, cL, bvx, ci, AW, K, hu, hH, cp, aa, dH, aW, hl, g, eE, eM, Jr, aiM, qR, oO, n, fr, qN, eW, ft, cU, h, dL, A, mR, aq, at, Xa, aAl, aCD, zl, B, Dw, AS, bvt, jx, aA, V, q, o, jF, lg, cei, aT, ae, Y, r, afq, lo, aDm, en, ik, pb, ig, bvy, AH, vi, Ke, aDy, iD, ad, hX, av, F, hD, uQ, atJ, d, KS, ff, cr, eZ, aF, xH, hp, iV, bz, bE, z, aCl, y, WX, hv); #c1(SEC14L3) c2(NP_001244307) c3(SEC14-like protein 3 isoform 2) c4(92116) c5(92117, 92118, 92119, 92120, 92121, 92122, 92123, 92124, 92125, 92126) c6( ) c7(bM, Ke, pf, bvt, aDy, if, oe, jx, iW, eb, fr, qN, V, ai, q, eW, hX, hD, h, azm, bK, hu, hH, uQ, hM, A, mR, Jl, Y, hl, cr, r, eZ, afq, lo, hp, gu, eE, pb, AH, cj, Da, Xa, y, WX, vi); #c1(SEC14L3) c2(NP_001244311) c3(SEC14-like protein 3 isoform 3) c4( ) c5(92127, 92128, 92129, 92130, 92131) c6( ) c7(bM, Ke, pf, bvt, aDy, if, oe, jx, iW, eb, fr, qN, V, ai, q, eW, hX, hD, h, azm, bK, hu, hH, uQ, hM, A, mR, Jl, Y, hl, cr, r, eZ, afq, lo, hp, gu, eE, pb, AH, cj, Da, Xa, y, WX, vi); #c1(SEC14L3) c2(NP_777635) c3(SEC14-like protein 3 isoform 1) c4(251352, 251353) c5(251354, 251355, 251356, 251357, 251358, 251359, 251360) c6( ) c7(bM, Ke, pf, bvt, aDy, if, oe, jx, iW, eb, fr, qN, V, ai, q, eW, hX, hD, h, azm, bK, hu, hH, uQ, hM, A, mR, Jl, Y, hl, cr, r, eZ, afq, lo, hp, gu, eE, pb, AH, cj, Da, Xa, y, WX, vi); #c1(SEC14L3) c2(XP_011528430) c3(SEC14-like protein 3 isoform X1) c4(323575) c5(323576, 323577, 323578, 323579, 323580, 323581) c6( ) c7(bM, Ke, pf, bvt, aDy, if, oe, jx, iW, eb, fr, qN, V, ai, q, eW, hX, hD, h, azm, bK, hu, hH, uQ, hM, A, mR, Jl, Y, hl, cr, r, eZ, afq, lo, hp, gu, eE, pb, AH, cj, Da, Xa, y, WX, vi); #c1(SEC16B) c2(NP_149118) c3(protein transport protein Sec16B) c4(227274, 227275, 227276) c5(227277, 227278, 227279, 227280, 227281, 227282, 227283, 227284, 227285, 227286, 227287, 227288, 227289, 227290, 227291, 227292, 227293, 227294) c6( ) c7(bL, bM, aq, v, nw, HU, fq, B); #c1(SEC23A) c2(NP_006355) c3(protein transport protein Sec23A) c4(164288, 164289, 164290, 164291) c5(164292, 164293, 164294, 164295, 164296, 164297, 164298, 164299, 164300, 164301, 164302, 164303, 164304, 164305, 164306, 164307, 164308, 164309, 164310, 164311, 164312, 164313, 164314, 164315, 164316, 164317, 164318, 164319, 164320, 164321, 164322, 164323, 164324, 164325, 164326, 164327, 164328) c6( ) c7(ae, ey, cCy); #c1(SEC23A) c2(XP_005267319) c3(protein transport protein Sec23A isoform X1) c4(280890, 280891, 280892, 280893) c5(280894, 280895, 280896, 280897, 280898, 280899, 280900, 280901, 280902, 280903, 280904) c6( ) c7(ae, ey, cCy); #c1(SEC23B) c2(NP_001166217) c3(protein transport protein Sec23B isoform 2) c4(68485) c5(68486, 68487, 68488, 68489, 68490, 68491) c6( ) c7(NU, byC, l, u, bzE, hC, adw, dn, oW, bsk, oY, pw, qD, dr); #c1(SEC23B) c2(NP_116780) c3(protein transport protein Sec23B isoform 1) c4(226462, 226463) c5(226464, 226465, 226466, 226467, 226468, 226469, 226470, 226471, 226472, 226473, 226474, 226475, 226476, 226477, 226478, 226479, 226480, 226481, 226482, 226483, 226484, 226485, 226486, 226487) c6( ) c7(NU, byC, l, u, bzE, hC, adw, dn, oW, bsk, oY, pw, qD, dr); #c1(SEC23IP) c2(NP_009121) c3(SEC23-interacting protein) c4(170857, 170858, 170859) c5(170860, 170861, 170862, 170863, 170864, 170865, 170866, 170867, 170868, 170869, 170870, 170871) c6( ) c7(dY, pN, bE, sP, EQ, aT, bB, aq, ey, K, m, g, fq, B); #c1(SEC23IP) c2(XP_005269526) c3(SEC23-interacting protein isoform X1) c4(282620) c5( ) c6( ) c7(dY, pN, bE, sP, EQ, aT, bB, aq, ey, K, m, g, fq, B); #c1(SEC23IP) c2(XP_005269527) c3(SEC23-interacting protein isoform X2) c4( ) c5(282621, 282622, 282623, 282624, 282625, 282626, 282627, 282628, 282629, 282630, 282631, 282632) c6( ) c7(dY, pN, bE, sP, EQ, aT, bB, aq, ey, K, m, g, fq, B); #c1(SEC24A) c2(NP_001239160) c3(protein transport protein Sec24A isoform 2) c4(88356) c5(88357, 88358, 88359, 88360, 88361, 88362) c6( ) c7(ac); #c1(SEC24A) c2(NP_068817) c3(protein transport protein Sec24A isoform 1) c4(210811) c5(210812, 210813, 210814, 210815, 210816, 210817) c6( ) c7(ac); #c1(SEC24B) c2(NP_001036199) c3(protein transport protein Sec24B isoform b) c4(34056) c5(34057, 34058, 34059, 34060, 34061, 34062) c6( ) c7(cCz); #c1(SEC24B) c2(NP_001287742) c3(protein transport protein Sec24B isoform c) c4(112753) c5(112754, 112755, 112756, 112757, 112758, 112759) c6( ) c7(cCz); #c1(SEC24B) c2(NP_006314) c3(protein transport protein Sec24B isoform a) c4(163922) c5(163923, 163924, 163925, 163926, 163927, 163928) c6( ) c7(cCz); #c1(SEC24C) c2(NP_940999) c3(protein transport protein Sec24C) c4(260302) c5(260303, 260304, 260305, 260306, 260307, 260308, 260309, 260310, 260311, 260312) c6( ) c7(cA, bk, af); #c1(SEC24C) c2(XP_011538683) c3(protein transport protein Sec24C isoform X1) c4(336430) c5(336431) c6( ) c7(cA, bK, af); #c1(SEC24C) c2(XP_011538684) c3(protein transport protein Sec24C isoform X2) c4(336432, 336433) c5(336434, 336435, 336436, 336437, 336438, 336439, 336440, 336441, 336442, 336443, 336444, 336445) c6( ) c7(cA, bK, af); #c1(SEC24C) c2(XP_011538686) c3(protein transport protein Sec24C isoform X3) c4( ) c5(336446, 336447, 336448, 336449, 336450, 336451, 336452, 336453, 336454, 336455) c6( ) c7(cA, bK, af); #c1(SEC31A) c2(NP_001070674) c3(protein transport protein Sec31A isoform 4) c4(34768) c5(34769, 34770, 34771, 34772, 34773, 34774, 34775, 34776, 34777, 34778) c6( ) c7(jh); #c1(SEC31A) c2(NP_001177978) c3(protein transport protein Sec31A isoform 5) c4(73254) c5(73255, 73256, 73257, 73258, 73259, 73260, 73261) c6( ) c7(jh); #c1(SEC31A) c2(NP_001287673) c3(protein transport protein Sec31A isoform 6) c4(112499) c5(112500, 112501, 112502, 112503, 112504, 112505, 112506, 112507, 112508, 112509, 112510, 112511, 112512, 112513, 112514, 112515, 112516, 112517, 112518, 112519, 112520) c6( ) c7(jh); #c1(SEC31A) c2(NP_001287674) c3(protein transport protein Sec31A isoform 7) c4(112521) c5(112522, 112523, 112524, 112525, 112526, 112527) c6( ) c7(jh); #c1(SEC31A) c2(NP_057295) c3(protein transport protein Sec31A isoform 2) c4(189945) c5(189946, 189947, 189948, 189949, 189950, 189951, 189952, 189953, 189954, 189955) c6( ) c7(jh); #c1(SEC31A) c2(XP_011530051) c3(protein transport protein Sec31A isoform X2) c4(325847, 325848, 325849, 325850, 325851, 325852) c5(325853, 325854, 325855, 325856, 325857, 325858, 325859, 325860, 325861, 325862, 325863, 325864, 325865, 325866, 325867, 325868, 325869, 325870, 325871, 325872, 325873, 325874, 325875, 325876, 325877, 325878, 325879) c6( ) c7(jh); #c1(SECG1A1) c2(NP_037468) c3(protein transport protein Sec61 subunit alpha isoform 1) c4(175657, 175658, 175659) c5(175660, 175661, 175662, 175663, 175664, 175665, 175666, 175667, 175668, 175669, 175670, 175671, 175672, 175673, 175674, 175675, 175676, 175677) c6( ) c7(J, K, Az, aj); #c1(SEC61G) c2(NP_055117) c3(protein transport protein Sec61 subunit gamma) c4(179045) c5(179046, 179047, 179048, 179049, 179050, 179051, 179052, 179053, 179054, 179055, 179056, 179057, 179058, 179059, 179060, 179061, 179062, 179063) c6( ) c7(eA, fd); #c1(SEC62) c2(NP_003253) c3(translocation protein SEC62) c4(136835) c5( ) c6( ) c7(ae, T, hd, eM, nB, n, K, bz, B, ad); #c1(SEC62) c2(XP_011511416) c3(translocation protein SEC62 isoform X1) c4(302049) c5(302050, 302051, 302052, 302053, 302054, 302055, 302056, 302057, 302058, 302059, 302060, 302061, 302062, 302063, 302064, 302065) c6( ) c7(ae, T, hd, eM, nB, n, K, bz, B, ad); #c1(SEC63) c2(NP_009145) c3(translocation protein SEC63 homolog) c4(171131, 171132) c5(171133, 171134, 171135, 171136, 171137, 171138, 171139) c6( ) c7(T, WQ, K, wB, hl); #c1(SEC63) c2(XP_011533701) c3(translocation protein SEC63 homolog isoform X1) c4(329784) c5(329785, 329786, 329787, 329788, 329789, 329790, 329791, 329792, 329793, 329794, 329795) c6( ) c7(T, WQ, K, wB, hl); #c1(SECISBP2) c2(NP_076982) c3(selenocysteine insertion sequence-binding protein 2 isoform 1) c4(214578, 214579) c5(214580, 214581, 214582, 214583, 214584, 214585, 214586, 214587, 214588, 214589, 214590, 214591, 214592) c6( ) c7(oj, r, hz, q, lM, cCA, xb, rL); #c1(SECISBP2) c2(XP_005252259) c3(selenocysteine insertion sequence-binding protein 2 isoform X7) c4( ) c5(271633) c6( ) c7(oj, r, hz, q, lM, cCA, xb, rL); #c1(SECISBP2) c2(XP_006717345) c3(selenocysteine insertion sequence-binding protein 2 isoform X1) c4(290953) c5(290954, 290955, 290956, 290957, 290958, 290959) c6( ) c7(oj, r, hz, q, lM, cCA, xb, rL); #c1(SECISBP2) c2(XP_011517302) c3(selenocysteine insertion sequence-binding protein 2 isoform X2) c4(310958) c5(310959, 310960, 310961, 310962, 310963) c6( ) c7(oj, r, hz, q, lM, cCA, xb, rL); #c1(SECISBP2) c2(XP_011517304) c3 (selenocysteine insertion sequence-binding protein 2 isoform X4) c4(310964) c5(310965, 310966, 310967, 310968, 310969, 310970, 310971, 310972) c6( ) c7(oj, r, hz, q, lM, cCA, xb, rl); #c1(SECISBP2L) c2(NP_001180418) c3(selenocysteine insertion sequence-binding protein 2-like isoform 1) c4(74031) c5(74032, 74033, 74034, 74035, 74036, 74037) c6( ) c7(T, bz); #c1(SECISBP2L) c2(NP_055516) c3 (selenocysteine insertion sequence-binding protein 2-like isoform 2) c4(181659) c5(181660, 181661, 181662, 181663, 181664, 181665) c6( ) c7(T, bz); #c1(SECTM1) c2(NP_002995) c3(secreted and transmembrane protein 1 precursor) c4(134254) c5(134255, 134256, 134257, 134258, 134259, 134260) c6( ) c7(bXu, aQu); #c1(SEL1L) c2(NP_001231913) c3 (protein sel-1 homolog 1 isoform 2 precursor) c4(87694) c5(87695, 87696, 87697, 87698, 87699, 87700) c6( ) c7(bL, T, aC, ads, ht, jh, hz, bak, aUz, ci, eA, ad, eJ, av, hd, aE, aT, hD, K, hu, dQ, ae, A, aW, ff, fe, akY, lo, kp, en, g, iV, mb, bz, mi, eM, Uq, it); #c1(SEL1L) c2(NP_005056) c3(protein sel-I homolog 1 isoform 1 precursor) c4(152489) c5(152490, 152491, 152492, 152493, 152494, 152495, 152496) c6( ) c7(bL, T, aC, ads, ht, jh, hz, bak, aUz, ci, eA, ad, eJ, av, hd, aE, aT, hD, K, hu, dQ, ae, A, aW, ff, fe, akY, lo, kp, en, g, iV, mb, bz, mi, eM, Uq, it); #c1(SEL1L) c2(XP_005268045) c3 (protein sel-I homolog 1 isoform X1) c4(281420) c5( ) c6( ) c7(bL, T, aC, ads, ht, jh, hz, bak, aUz, ci, eA, ad, eJ, av, hd, aE, aT, HD, K, hu, dQ, ae, A, aW, ff, fe, akY, lo, kp, en, g, iV, mb, bz, mi, eM, Uq, it); #c1(SEL1L) c2(XP_005268046) c3(protein sel-1 homolog 1 isoform X2) c4( ) c5(281421, 281422, 281423, 281424, 281425) c6( ) c7(bL, T, aC, ads, ht, jh, hz, bak, aUz, ci, eA, ad, eJ, av, hd, aE, aT, hD, K, hu, dQ, ae, A, aW, ff, fe, akY, lo, kp, en, g, iV, mb, bz, mi, eM, Uq, it); #c1(SELE) c2(NP_000441) c3(E-selectin precursor) c4(8988, 8989, 8990, 8991, 8992) c5(8993, 8994, 8995, 8996, 8997, 8998, 8999, 9000, 9001, 9002, 9003, 9004, 9005, 9006, 9007, 9008, 9009, 9010, 9011, 9012, 9013, 9014, 9015, 9016, 9017, 9018, 9019, 9020, 9021, 9022, 9023, 9024, 9025, 9026, 9027, 9028, 9029, 9030, 9031, 9032, 9033, 9034, 9035, 9036, 9037, 9038) c6( ) c7(bM, T, aC, fR, ea, e, bB, cL, fo, ci, aY, Tz, Hr, wa, dY, aE, M, dV, K, ass, aW, asD, hl, fc, zC, g, eE, uC, ou, lt, vy, eg, qR, dD, aw, aj, fr, eh, eW, ec, lG, dL, ey, ux, cY, A, MD, Cw, PX, fJ, fe, cM, ar, kT, W, bPs, uz, fL, Pr, B, bL, eR, AL, wL, fG, oe, rf, aA, cR, cz, fF, v, j, bX, V, bpW, q, o, J, qp, aT, cy, qG, aAD, ae, af, qk, bA, eu, NE, oV, Y, ev, r, en, pb, ck, nM, ac, c, aQd, lV, AH, cCB, hO, wR, mf, cS, byK, bt, gV, sY, aFe, qb, aD, iW, jh, OR, dG, eq, jE, mC, ad, KL, hX, av, hd, LA, hD, JK, ji, vl, yF, uQ, hG, cH, cW, ff, gC, eZ, aF, hp, Fq, qa, D, pc, cCC, bE, aS, agl, ei, XO, lC, X, y); #c1(SELENBP1) c2(NP_001245217) c3(selenium-binding protein 1 isoform 2) c4(92744) c5(92745, 92746, 92747, 92748, 92749, 92750) c6( ) c7(bL, T, pf, e, aA, cz, fd, aj, v, j, q, J, ad, av, axc, aT, afV, bK, K, ae, Nr, aW, r, fQ, gl, po, g, Eg, t, Nq, vK, z, Gs, m, ei, B); #c1(SELENBP1) c2(NP_003935) c3(selenium-binding protein 1 isoform 1) c4(142310) c5(142311, 142312, 142313, 142314, 142315, 142316, 142317, 142318, 142319, 142320) c6( ) c7(bL, T, pf, e, aA, cz, fd, aj, v, j, q, J, ad, av, axc, aT, afV, bK, K, ae, Nr, aW, r, fQ, gl, po, g, Eg, t, Nq, vK, z, Gs, m, ei, B); #c1(SELENBP1) c2(XP_011508412) c3(selenium-binding protein 1 isoform X1) c4(299134) c5(299135, 299136) c6( ) c7(bL, T, pf, e, aA, cz, fd, aj, v, j, q, J, ad, av, axc, aT, afV, bK, K, ae, Nr, aW, r, fQ, gl, po, g, Eg, t, Nq, vK, z, Gs, m, ei, B); #c1(SELK) c2(NP_067060) c3(selenoprotein K) c4(208781, 208782) c5(208783, 208784, 208785, 208786, 208787, 208788, 208789, 208790, 208791, 208792, 208793, 208794) c6( ) c7(ae, RT, ad); #c1(SELL) c2(NP_000646) c3(L-selectin precursor) c4(14150) c5(14151, 14152, 14153, 14154, 14155, 14156) c6( ) c7(bM, T, fR, ea, e, fo, ci, aFV, aE, aoL, Ds, K, hH, asD, zC, uC, vy, cj, dD, aw, aDT, aj, ct, ai, KH, FN, lG, h, ey, A, fe, ar, dB, bm, at, bL, Dw, dA, eR, aA, cR, cz, j, C, V, J, qp, cy, bA, iU, xR, oV, Y, ck, nM, ac, c, aQd, iM, hc, dy, bt, qb, aD, iW, jh, bS, eq, EA, wD, dP, cCD, HD, ji, uX, je, eZ, hp, iV, Z, dr, es, ell, bE, XO, y, fh); #c1(SELO) c2(NP_113642) c3(selenoprotein 0) c4(221893) c5(221894, 221895, 221896, 221897, 221898, 221899) c6( ) c7(LU); #c1(SELP) c2(NP_002996) c3(P-selectin precursor) c4(134261, 134262, 134263) c5(134264, 134265, 134266, 134267, 134268, 134269, 134270, 134271, 134272, 134273, 134274, 134275, 134276, 134277, 134278, 134279, 134280, 134281, 134282, 134283, 134284, 134285, 134286, 134287, 134288, 134289) c6( ) c7(bM, T, ea, e, al, fd, cL, fo, ci, dY, vV, Bc, M, K, dQ, DY, hm, aW, mQ, hl, eE, uC, nB, vy, cj, wx, dD, aw, aj, cd, eW, ec, h, lG, ey, cY, A, sp, EU, fQ, cM, ar, Fi, pg, bL, eR, AL, mr, zk, rf, cz, v, j, C, bX, V, eA, o, J, rS, aT, cy, af, eu, bA, iU, NE, r, pb, ck, ac, wO, hc, bSs, fD, sY, bRw, qb, auq, eq, dZ, av, qh, JK, ji, lQ, bGD, vl, sj, d, cH, eZ, kD, bRq, qa, pc, bE, aka, aFb, gZ, MJ, aS, ME, lC, xg, iu, y); #c1(SELP) c2(XP_005245496) c3(P-selectin isoform X2) c4(266610) c5(266611, 266612, 266613, 266614, 266615, 266616) c6( ) c7(bM, T, ea, e, al, fd, cL, fo, ci, dY, vV, Bc, M, K, dQ, DY, hm, aW, mQ, hl, eE, uC, nB, vy, cj, wx, dD, aw, aj, cd, eW, ec, h, lG, ey, cY, A, sp, EU, fQ, cM, ar, Fi, pg, bL, eR, AL, mr, zk, rf, cz, v, j, C, bX, V, eA, o, J, rS, aT, cy, af, eu, bA, iU, NE, r, pb, ck, ac, wO, hc, bSs, fD, sY, bRw, qb, auq, eq, dZ, av, qh, JK, ji, lQ, bGD, vl, sj, d, cH, eZ, kD, bRq, qa, pc, bE, aka, aFb, gZ, MJ, aS, ME, lC, xg, iu, y); #c1(SELP) c2(XP_005245497) c3(P-selectin isoform X3) c4(266617, 266618, 266619, 266620, 266621) c5(266622, 266623, 266624, 266625, 266626, 266627, 266628, 266629, 266630, 266631, 266632, 266633, 266634, 266635, 266636, 266637, 266638, 266639, 266640, 266641, 266642, 266643, 266644, 266645, 266646, 266647, 266648, 266649, 266650, 266651, 266652, 266653, 266654, 266655, 266656, 266657, 266658, 266659, 266660, 266661, 266662, 266663, 266664) c6( ) c7(bM, T, ea, e, al, fd, cL, fo, ci, dY, vV, Bc, M, K, dQ, DY, hm, aW, mQ, hl, eE, uC, nB, vy, cj, wx, dD, aw, aj, cd, eW, ec, h, lG, ey, cY, A, sp, EU, fQ, cM, ar, Fi, pg, bL, eR, AL, mr, zk, rf, cz, v, j, C, bX, V, eA, o, J, rS, aT, cy, af, eu, bA, iU, NE, r, pb, ck, ac, wO, hc, bSs, fD, sY, bRw, qb, auq, eq, dZ, av, qh, JK, ji, lQ, bGD, vl, sj, d, cH, eZ, kD, bRq, qa, pc, bE, aka, aFb, gZ, MJ, aS, ME, lC, xg, iu, y); #c1(SELPLG) c2(NP_001193538) c3(P-selectin glycoprotein ligand 1 isoform 1 precursor) c4(82253, 82254) c5( ) c6( ) c7(T, dD, ea, iW, aw, ez, fd, aj, v, j, eq, PJ, V, eW, J, aDF, qh, M, cy, ji, K, hm, OV, xR, cr, lo, eE, axp, ck, ell, ac, cj, qq, eo, XO, Gp); #c1(SELPLG) c2(XP_005269133) c3(P-selectin glycoprotein ligand 1 isoform X1) c4(282339) c5(282340, 282341, 282342, 282343, 282344, 282345, 282346, 282347, 282348, 282349, 282350, 282351, 282352, 282353, 282354, 282355, 282356, 282357) c6( ) c7(T, dD, ea, iW, aw, ez, fd, aj, v, j, eq, PJ, V, eW, J, aDF, qh, M, cy, ji, K, hm, OV, xR, cr, lo, eE, axp, ck, eU, ac, cj, qq, eo, XO, Gp); #c1(SELT) c2(NP_057359) c3(selenoprotein T precursor) c4(190334) c5(190335, 190336, 190337, 190338, 190339, 190340) c6( ) c7(ey); #c1(SEMA3A) c2(XP_006715902) c3(semaphorin-3A isoform X1) c4(289903, 289904) c5(289905, 289906, 289907, 289908, 289909, 289910, 289911, 289912, 289913, 289914, 289915, 289916, 289917, 289918, 289919, 289920, 289921, 289922, 289923, 289924, 289925, 289926, 289927, 289928, 289929, 289930) c6(289931, 289932, 289933, 289934, 289935, 289936) c7(T, dt, aC, Ol, oe, e, rf, fd, aj, cCF, ci, V, eA, ad, J, yn, av, yt, fm, aT, h, cCE, ae, af, cY, aW, Y, cr, ahJ, VO, c, ln, vy, wR, UF, hS); #c1(SEMA3A) c2(XP_011514037) c3(semaphorin-3A isoform X2) c4(306461, 306462) c5(306463, 306464, 306465, 306466, 306467, 306468, 306469, 306470, 306471, 306472) c6(306473, 306474) c7(T, dt, aC, Ol, oe, e, rf, fd, aj, cCF, ci, V, eA, ad, J, yn, av, yt, fm, aT, h, cCE, ae, af, cY, aW, Y, cr, ahJ, VO, c, ln, vy, wR, UF, hS); #c1(SEMA3B) c2(NP_001276989) c3(semaphorin-38 isoform 1 precursor) c4(108914, 108915, 108916) c5(108917, 108918, 108919, 108920, 108921, 108922, 108923, 108924, 108925, 108926, 108927, 108928, 108929, 108930, 108931, 108932, 108933, 108934, 108935, 108936, 108937, 108938) c6(108939, 108940, 108941, 108942) c7(T, eA, ad, av, ft, jz, Ok, io, ae, aa, d, aq, g, cm, t, bz, bP, eM, hL, m, it, B, hS); #c1(SEMA3C) c2(NP_006370) c3(semaphorin-3C precursor) c4(164417, 164418, 164419) c5(164420, 164421, 164422, 164423, 164424, 164425, 164426, 164427, 164428, 164429, 164430, 164431, 164432, 164433, 164434, 164435, 164436, 164437, 164438, 164439, 164440) c6(164441, 164442, 164443, 164444, 164445, 164446) c7(ae, T, iu, yt, aw, ff, aT, aA, Hr, aF, ahL, en, eA, y, ahE, B, ad); #c1(SEMA3C) c2(XP_005250170) c3(semaphorin-3C isoform X1) c4( ) c5(270150, 270151, 270152, 270153, 270154, 270155, 270156, 270157) c6(270158, 270159) c7(ae, T, iu, yt, aw, ff, aT, aA, Hr, aF, ahL, en, eA, y, ahE, B, ad); #c1(SEMA3D) c2(NP_689967) c3(semaphorin-3D precursor) c4(244620) c5(244621, 244622, 244623, 244624, 244625, 244626, 244627, 244628) c6(244629, 244630) c7(W, gv, ahL, hH, ahE, eA); #c1(SEMA3E) c2(NP_001171600) c3(semaphorin-3E isoform 2) c4( ) c5(70712, 70713, 70714, 70715, 70716) c6( ) c7(T, wL, Y, avQ, ae, ad, cp); #c1(SEMA3E) c2(NP_036563) c3(semaphorin-3E isoform 1 precursor) c4(174418, 174419, 174420) c5(174421, 174422, 174423, 174424, 174425, 174426, 174427, 174428, 174429, 174430, 174431, 174432, (74433) c6( ) c7(T, wL, Y, avQ, ae, ad, cp); #c1(SEMA3F) c2(NP_004177) c3(semaphorin-3F precursor) c4(144100) c5(144101, 144102, 144103, 144104, 144105, 144106, 144107, 144108) c6(144109, 144110) c7(ae, T, ft, av, ac, VO, bE, aW, Ls, aw, hL, r, aT, bB, cCG, bz, q, ad); #c1(SEMA3F) c2(XP_006713353) c3(semaphorin-3F isoform X5) c4( ) c5(287155, 287156, 287157, 287158, 287159, 287160) c6( ) c7(ae, T, ft, av, ac, VO, bE, aW, Ls, aw, hL, r, aT, bB, cCG, bz, q, ad); #c1(SEMA3F) c2(XP_011532301) c3(semaphorin-3F isoform X2) c4(328445, 328446, 328447, 328448) c5(328449, 328450, 328451, 328452, 328453, 328454, 328455, 328456, 328457, 328458, 328459, 328460, 328461, 328462, 328463, 328464, 328465, 328466) c6( ) c7(ae, T, ft, av, ac, VO, bE, aW, Ls, aw, hL, r, aT, bB, cCG, bz, q, ad); #c1(SEMA3G) c2(NP_064548) c3(semaphorin-3G precursor) c4(202926) c5(202927, 202928, 202929, 202930, 202931, 202932, 202933, 202934) c6(202935, 202936) c7(eA, hS); #c1(SEMA4A) c2(NP_001180231) c3(semaphorin-4A isoform 2) c4(73466) c5(73467, 73468, 73469, 73470, 73471, 73472, 73473, 73474) c6( ) c7(Kd, bM, lR, Ke, eM, z, eE, U, cCl, nw, eo, cCH, oQ, fD, V, nC); #c1(SEMA4A) c2(XP_011508173) c3(semaphorin-4A isoform X2) c4(298732) c5(298733) c6( ) c7(Kd, bM, lR, Ke, eM, z, eE, U, cCl, nw, eo, cCH, oQ, fD, V, nC); #c1(SEMA4A) c2(XP_011508175) c3(semaphorin-4A isoform X1) c4(298734, 298735, 298736, 298737) c5(298738, 298739, 298740, 298741, 298742, 298743, 298744, 298745, 298746, 298747, 298748, 298749, 298750, 298751, 298752, 298753, 298754, 298755, 298756, 298757, 298758, 298759, 298760, 298761, 298762, 298763, 298764, 298765, 298766, 298767, 298768, 298769, 298770, 298771, 298772, 298773, 298774, 298775) c6( ) c7(Kd, bM, lR, Ke, eM, z, eE, U, cCl, nw, eo, cCH, oQ, fD, V, nC); #c1(SEMA4A) c2(XP_011508180) c3(semaphorin-4A isoform X3) c4( ) c5(298776, 298777, 298778, 298779, 298780) c6( ) c7(Kd, bM, lR, Ke, eM, z, eE, U, cCl, nw, eo, cCH, oQ, fD, V, nC); #c1(SEMA4B) c2(NP_064595) c3(semaphorin-48 precursor) c4(203162, 203163, 203164, 203165, 203166) c5(203167, 203168, 203169, 203170, 203171, 203172, 203173, 203174, 203175, 203176, 203177, 203178, 203179, 203180, 203181, 203182, 203183, 203184, 203185, 203186, 203187, 203188, 203189, 203190, 203191, 203192, 203193, 203194, 203195, 203196, 203197, 203198, 203199, 203200) c6( ) c7(bz, iu); #c1(SEMA4B) c2(XP_011519462) c3(semaphorin-48 isoform X1) c4( ) c5(314130) c6( ) c7(bz, iu); #c1(SEMA4D) c2(XP_005251711) c3(semaphorin-4D isoform X1) c4(271066, 271067, 271068, 271069, 271070) c5(271071, 271072, 271073, 271074, 271075, 271076, 271077, 271078, 271079, 271080, 271081, 271082, 271083, 271084, 271085, 271086, 271087, 271088) c6( ) c7(bM, cT, T, aE, af, yE, Y, ff, cj, aj, kp, ci, en, DY, ik, ae, cd, J, ad); #c1(SEMA4D) c2(XP_011516437) c3(semaphorin-4D isoform X2) c4(309650, 309651) c5(309652, 309653, 309654, 309655, 309656, 309657, 309658, 309659, 309660, 309661, 309662, 309663, 309664, 309665, 309666, 309667) c6( ) c7(bM, cT, T, aE, af, yE, Y, ff, cj, aj, kp, ci, en, DY, ik, ae, cd, J, ad); #c1(SEMA4D) c2(XP_011516439) c3(semaphorin-4D isoform X4) c4( ) c5(309668, 309669, 309670, 309671, 309672, 309673, 309674, 309675, 309676, 309677, 309678, 309679, 309680, 309681, 309682, 309683, 309684, 309685, 309686, 309687, 309688, 309689, 309690, 309691) c6( ) c7(bM, cT, T, aE, af, yE, Y, ff, cj, aj, kp, ci, en, DY, ik, ae, cd, J, ad); #c1(SEMA4F) c2(NP_001258590) c3(semaphorin-4F isoform 2 precursor) c4(96167) c5(96168, 96169, 96170, 96171, 96172) c6( ) c7(n, ae, ad); #c1(SEMA4F) c2(NP_004254) c3(semaphorin-4F isoform 1 precursor) c4(144631) c5(144632, 144633, 144634, 144635, 144636, 144637, 144638) c6( ) c7(n, ae, ad); #c1(SEMA4G) c2(NP_001190173) c3(semaphorin-4G isoform 2 precursor) c4(80299) c5(80300, 80301, 80302, 80303, 80304, 80305, 80306, 80307, 80308, 80309, 80310, 80311) c6( ) c7(es, fr); #c1(SEMA4G) c2(NP_060363) c3(semaphorin-4G isoform 1 precursor) c4(195592, 195593) c5(195594, 195595, 195596, 195597, 195598, 195599, 195600, 195601, 195602, 195603, 195604, 195605, 195606, 195607, 195608, 195609, 195610, 195611) c6( ) c7(es, fr); #c1(SEMA4G) c2(XP_005270067) c3(semaphorin-4G isoform X2) c4(283081, 283082, 283083) c5(283084, 283085, 283086, 283087, 283088, 283089, 283090, 283091, 283092, 283093) c6( ) c7(es, fr); #c1(SEMA5A) c2(NP_003957) c3(semaphorin-5A precursor) c4(142444, 142445) c5(142446, 142447, 142448, 142449, 142450, 142451, 142452, 142453, 142454, 142455, 142456, 142457) c6( ) c7(T, aC, dY, aE, aYJ, oe, aw, aA, By, aF, nA, an, as, eA, bN); #c1(SEMA5A) c2(XP_011512461) c3(semaphorin-5A isoform X2) c4( ) c5(303142, 303143) c6( ) c7(T, aC, dY, aE, aYJ, oe, aw, aA, By, aF, nA, an, as, eA, bN); #c1(SEMA5B) c2(NP_001026872) c3(semaphorin-58 isoform 1) c4(29743) c5(29744, 29745, 29746, 29747, 29748, 29749, 29750, 29751, 29752, 29753, 29754, 29755, 29756, 29757, 29758, 29759, 29760, 29761, 29762, 29763) c6( ) c7(fr, ht); #c1(SEMA5B) c2(NP_001243276) c3(semaphorin-58 isoform 2) c4(90349, 90350, 90351, 90352) c5(90353, 90354, 90355, 90356, 90357, 90358, 90359, 90360) c6( ) c7(fr, ht); #c1(SEMA5B) c2(NP_001243277) c3(semaphorin-5B isoform 3 precursor) c4(90361) c5(90362, 90363, 90364, 90365, 90366, 90367) c6( ) c7(fr, ht); #c1(SEMA5B) c2(XP_011511219) c3(semaphorin-5B isoform X1) c4(301697) c5(301698, 301699, 301700, 301701, 301702, 301703, 301704, 301705) c6( ) c7(fr, ht); #c1(SEMA6A) c2(NP_001287709) c3(semaphorin-6A isoform 1 precursor) c4(112582, 112583, 112584, 112585) c5(112586, 112587, 112588, 112589, 112590, 112591) c6( ) c7(bM, T, os, ea, if, al, fd, bB, VV, x, aE, fm, M, aU, iv, K, hu, DY, aiV, dH, aW, ve, uC, cm, eM, Jr, nB, uo, cCL, dW, k, m, yA, aTe, dD, lo, ht, n, aj, fr, qN, cd, ai, eW, cCK, h, he, cY, A, agl, hL, hs, hR, at, HB, it, B, cn, jv, fM, dA, lH, jx, aA, cz, arw, sl, Eh, q, J, er, eA, aT, cy, ae, af, Y, r, en, cl, t, nC, ik, pb, ck, kP, c, aGi, au, bt, rA, ao, ha, oZ, bS, eq, cCJ, ad, qh, ji, vP, lR, ff, wc, cr, eZ, gl, Ej, aF, bz, dr, aYt, ek, gk, RV, bE, uR, ll, dX, y, fh); #c1(SEMA6A) c2(NP_065847) c3(semaphorin-6A isoform 2 precursor) c4(205833) c5(205834, 205835, 205836, 205837, 205838, 205839, 205840, 205841, 205842, 205843, 205844, 205845, 205846, 205847, 205848, 205849) c6( ) c7(bM, T, os, ea, if, al, fd, bB, W, x, aE, fm, M, aU, iv, K, hu, DY, aiV, dH, aW, ve, uC, cm, eM, Jr, nB, uo, cCL, dW, k, m, yA, aTe, dD, lo, ht, n, aj, fr, qN, cd, ai, eW, cCK, h, he, cY, A, agl, hL, hs, hR, at, HB, it, B, cn, jv, fM, dA, lH, jx, aA, cz, arw, sl, Eh, q, J, er, eA, aT, cy, ae, af, Y, r, en, cl, t, nC, ik, pb, ck, kP, c, aGi, au, bt, rA, ao, ha, oZ, bS, eq, cCJ, ad, qh, ji, vP, lR, ff, wc, cr, eZ, gl, Ej, aF, bz, dr, aYt, ek, gk, RV, bE, uR, ll, dX, y, fh); #c1(SEMA6A) c2(XP_005272099) c3(semaphorin-6A isoform X2) c4(283675) c5(283676, 283677, 283678, 283679, 283680, 283681, 283682, 283683, 283684, 283685, 283686, 283687, 283688, 283689, 283690, 283691, 283692, 283693, 283694, 283695, 283696, 283697, 283698, 283699) c6( ) c7(bM, T, os, ea, if, al, fd, bB, VV, x, aE, fm, M, aU, iv, K, hu, DY, aiV, dH, aW, ve, uC, cm, eM, Jr, nB, uo, cCL, dW, k, m, yA, aTe, dD, lo, ht, n, aj, fr, qN, cd, ai, eW, cCK, h, he, cY, A, agl, hL, hs, hR, at, HB, it, B, cn, jv, fM, dA, lH, jx, aA, cz, arw, sl, Eh, q, J, er, eA, aT, cy, ae, af, Y, r, en, cl, t, nC, ik, pb, ck, kP, c, aGi, au, bt, rA, ao, ha, oZ, bS, eq, cCJ, ad, qh, ji, vP, lR, ff, wc, cr, eZ, gl, Ej, aF, bz, dr, aYt, ek, gk, RV, bE, uR, ll, dX, y, fh); #c1(SEMA6A) c2(XP_006714726) c3(semaphorin-6A isoform X1) c4(288055) c5( ) c6( ) c7(bM, T, os, ea, if, al, fd, bB, VV, x, aE, fm, M, aU, iv, K, hu, DY, aiV, dH, aW, ve, uC, cm, eM, Jr, nB, uo, cCL, dW, k, m, yA, aTe, dD, lo, ht, n, aj, fr, qN, cd, ai, eW, cCK, h, he, cY, A, agl, hL, hs, hR, at, HB, it, B, cn, jv, fM, dA, lH, jx, aA, cz, arw, sl, Eh, q, J, er, eA, aT, cy, ae, af, Y, r, en, cl, t, nC, ik, pb, ck, kP, c, aGi, au, bt, rA, ao, ha, oZ, bS, eq, cCJ, ad, qh, ji, vP, lR, ff, wc, cr, eZ, gl, Ej, af, bz, dr, aYt, ek, gk, RV, bE, uR, ll, dX, y, fh); #c1(SEMA6B) c2(NP_115484) c3(semaphorin-GB precursor) c4(222868, 222869) c5(222870, 222871, 222872, 222873, 222874, 222875, 222876, 222877, 222878, 222879, 222880, 222881, 222882, 222883, 222884, 222885) c6( ) c7(d, im); #c1(SEMA6D) c2(NP_001185928) c3(semaphorin-6D isoform 1 precursor) c4(76776, 76777) c5(76778, 76779, 76780, 76781, 76782, 76783, 76784, 76785, 76786, 76787, 76788, 76789, 76790, 76791, 76792, 76793, 76794, 76795, 76796, 76797) c6( ) c7(eR, nO, fe, an, aY, lB, fq); #c1(SEMA6D) c2(NP_705872) c3(semaphorin-GD isoform 5 precursor) c4(246365) c5(246366, 246367, 246368, 246369, 246370, 246371, 246372, 246373, 246374, 246375) c6( ) c7(eR, nO, fe, an, aY, lB, fq); #c1(SEMA6D) c2(XP_005254744) c3(semaphorin-6D isoform X4) c4(273309, 273310, 273311) c5(273312, 273313, 273314, 273315, 273316, 273317, 273318, 273319, 273320, 273321) c6( ) c7(eR, nO, fe, an, aY, lB, fq); #c1(SEMA6D) c2(XP_005254746) c3(semaphorin-6D isoform X8) c4(273322, 273323) c5(273324, 273325, 273326, 273327, 273328, 273329) c6( ) c7(eR, nO, fe, an, aY, lB, fq); #c1(SEMA6D) c2(XP_011520377) c3(semaphorin-60 isoform X6) c4( ) c5(315199, 315200, 315201, 315202, 315203) c6( ) c7(eR, nO, fe, an, aY, lB, fq); #c1(SEMA6D) c2(XP_011520380) c3(semaphorin-6D isoform X9) c4(315204) c5(315205, 315206, 315207, 315208, 315209, 315210) c6( ) c7(eR, nO, fe, an, aY, lB, fq); #c1(SEMA6D) c2(XP_011520381) c3(semaphorin-6D isoform X3) c4(315211) c5(315212, 315213, 315214, 315215, 315216, 315217) c6( ) c7(eR, nO, fe, an, aY, lB, fq); #c1(SEMA6D) c2(XP_011520383) c3(semaphorin-6D isoform X2) c4(315218) c5(315219, 315220, 315221, 315222, 315223, 315224) c6( ) c7(eR, nO, fe, an, aY, lB, fq); #c1(SEMA6D) c2(XP_011520384) c3(semaphorin-6D isoform X1) c4( ) c5(315225, 315226, 315227, 315228, 315229, 315230, 315231, 315232, 315233, 315234, 315235, 315236, 315237) c6( ) c7(eR, nO, fe, an, aY, lB, fq); #c1(SEMA7A) c2(NP_001139501) c3(semaphorin-7A isoform 2 preproprotein) c4(59434) c5(59435, 59436, 59437, 59438, 59439, 59440) c6( ) c7(T, Y, fG, bW, Du, yq, lR, cCM, pb, yn, UF); #c1(SEMA7A) c2(NP_001139502) c3(semaphorin-7A isoform 3) c4(59441) c5( ) c6( ) c7(T, Y, fG, bW, Du, yq, lR, cCM, pb, yn, UF); #c1(SEMA7A) c2(NP_003603) c3(semaphorin-7A isoform 1 preproprotein) c4(139516) c5(139517, 139518, 139519, 139520, 139521, 139522, 139523, 139524, 139525, 139526, 139527, 139528) c6( ) c7(T, Y, fG, bW, Du, yq, lR, cCM, pb, yn, UF); #c1(SEMG1) c2(NP_002998) c3(semenogelin-1 preproprotein) c4(134290, 134291) c5(134292, 134293, 134294, 134295, 134296, 134297, 134298, 134299, 134300, 134301, 134302, 134303) c6( ) c7(T, iU, MD, Me, r, cj, vH, fd, kp, fr, bu, bt, jJ, q); #c1(SEMG2) c2(NP_002999) c3(semenogelin-2 precursor) c4(134304) c5(134305, 134306, 134307, 134308, 134309, 134310) c6( ) c7(T); #c1(SENP1) c2(NP_001254524) c3(sentrin-specific protease 1) c4(94591, 94592) c5( ) c6( ) c7(ck, ae, eW, nB, bE, aW, vT, aT, bB, PQ, bz, y, kS, pi, ad); #c1(SENP1) c2(XP_006719424) c3(sentrin-specific protease 1 isoform X1) c4(292468) c5(292469, 292470, 292471, 292472) c6( ) c7(ck, ae, eW, nB, bE, aW, vT, aT, bB, PQ, bz, y, kS, pi, ad); #c1(SENP1) c2(XP_011536546) c3(sentrin-specific protease 1 isoform X2) c4( ) c5(334004, 334005, 334006, 334007, 334008) c6( ) c7(ck, ae, eW, nB, bE, aW, vT, aT, bB, PQ, bz, y, kS, pi, ad); #c1(SENP1) c2(XP_011536547) c3(sentrin-specific protease 1 isoform X3) c4( ) c5(334009, 334010, 334011, 334012, 334013, 334014, 334015, 334016, 334017, 334018, 334019, 334020, 334021, 334022, 334023, 334024, 334025, 334026, 334027, 334028, 334029, 334030, 334031) c6( ) c7(ck, ae, eW, nB, bE, aW, vT, aT, bB, PQ, bz, y, kS, pi, ad); #c1(SENP2) c2(NP_067640) c3(sentrin-specific protease 2) c4(209152, 209153, 209154) c5(209155, 209156, 209157, 209158, 209159, 209160, 209161, 209162, 209163, 209164, 209165, 209166, 209167, 209168, 209169) c6( ) c7(bD, eL, acl, dL); #c1(SENP2) c2(XP_005247748) c3(sentrin-specific protease 2 isoform X2) c4(267957) c5(267958, 267959, 267960, 267961, 267962, 267963, 267964, 267965, 267966, 267967, 267968) c6( ) c7(bD, eL, acl, dL); #c1(SENP3) c2(NP_056485) c3(sentrin-specific protease 3) c4(187722, 187723) c5(187724, 187725, 187726, 187727, 187728, 187729, 187730, 187731, 187732, 187733, 187734, 187735) c6( ) c7(cy, T, kS, K, ev); #c1(SENP5) c2(NP_689912) c3(sentrin-specific protease 5) c4(244343) c5(244344, 244345, 244346, 244347, 244348, 244349) c6( ) c7(ff, en); #c1(SENP6) c2(NP_001093879) c3(sentrin-specific protease 6 isoform 2) c4(40475, 40476) c5(40477, 40478, 40479, 40480, 40481, 40482) c6( ) c7(T, K, C); #c1(SENP6) c2(NP_001291721) c3(sentrin-specific protease 6 isoform 3) c4(116383) c5(116384, 116385, 116386, 116387, 116388) c6( ) c7(T, K, C); #c1(SENP6) c2(NP_056386) c3(sentrin-specific protease 6 isoform 1) c4(187385, 187386) c5(187387, 187388, 187389, 187390, 187391, 187392, 187393, 187394, 187395, 187396, 187397, 187398) c6( ) c7(T, K, C); #c1(SENP6) c2(XP_011534049) c3(sentrin-specific protease 6 isoform X4) c4( ) c5(330217, 330218, 330219, 330220, 330221, 330222, 330223) c6( ) c7(T, K, C); #c1(SENP8) c2(NP_001165580) c3(sentrin-specific protease 8) c4( ) c5(67802, 67803, 67804, 67805, 67806, 67807, 67808, 67809, 67810, 67811, 67812, 67813, 67814, 67815) c6( ) c7(bz, AH, cCN, aik); #c1(SENP8) c2(XP_011519518) c3(sentrin-specific protease 8 isoform X2) c4(314173) c5(314174, 314175, 314176, 314177) c6( ) c7(bz, AH, cCN, aik); #c1(37135) c2(NP_443070) c3(septin-1) c4(229217) c5(229218, 229219, 229220, 229221, 229222, 229223, 229224, 229225, 229226, 229227) c6( ) c7(uY, T, ff, en); #c1(37500) c2(NP_001008492) c3(septin-2 isoform a) c4(23215, 23216, 23217) c5(23218, 23219, 23220, 23221, 23222, 23223, 23224, 23225, 23226, 23227, 23228, 23229, 23230, 23231, 23232, 23233, 23234, 23235, 23236, 23237, 23238, 23239, 23240, 23241, 23242, 23243, 23244, 23245, 23246, 23247, 23248, 23249, 23250, 23251, 23252, 23253, 23254, 23255, 23256, 23257, 23258, 23259, 23260, 23261, 23262, 23263, 23264) c6( ) c7(dB, eZ, at, dL, K, as, bw, bt, pb); #c1(37500) c2(NP_001269901) c3(septin-2 isoform b) c4(103108) c5(103109, 103110, 103111, 103112, 103113, 103114, 103115) c6( ) c7(dB, eZ, at, dL, K, as, bw, bt, pb); #c1(37500) c2(NP_001269902) c3(septin-2 isoform c) c4(103116) c5( ) c6( ) c7(dB, eZ, at, dL, K, as, bw, bt, pb); #c1(37500) c2(XP_006712610) c3(septin-2 isoform X2) c4(286732, 286733, 286734, 286735) c5(286736, 286737, 286738, 286739, 286740, 286741, 286742, 286743, 286744, 286745) c6( ) c7(dB, eZ, at, dL, K, as, bw, bt, pb); #c1(37500) c2(XP_011509534) c3(septin-2 isoform X3) c4(300120) c5(300121, 300122, 300123, 300124, 300125, 300126) c6( ) c7(dB, eZ, at, dL, K, as, bw, bt, pb); #c1(37865) c2(NP_061979) c3(neuronal-specific septin-3 isoform B) c4(201575, 201576, 201577, 201578) c5(201579, 201580, 201581, 201582, 201583, 201584, 201585, 201586, 201587, 201588, 201589, 201590, 201591, 201592, 201593, 201594, 201595, 201596, 201597, 201598, 201599, 201600, 201601) c6( ) c7(af, jN); #c1(37865) c2(NP_663786) c3 (neuronal-specific septin-3 isoform A) c4(241598, 241599) c5(241600, 241601, 241602, 241603, 241604, 241605, 241606, 241607, 241608, 241609, 241610, 241611, 241612) c6( ) c7(af, jN); #c1(38231) c2(NP_001185642) c3(septin-4 isoform 4) c4(76015) c5(76016, 76017, 76018, 76019, 76020, 76021, 76022) c6( ) c7(T, ad, XX, x, aT, K, ae, azh, ago, d, B, aW, ff, hl, en, as, ik, csw, U, k, hE, X); #c1(38231) c2(NP_001243711) c3(septin-4 isoform 5) c4(91519) c5(91520, 91521, 91522, 91523, 91524, 91525, 91526) c6( ) c7(T, ad, XX, x, aT, K, ae, azh, ago, d, B, aW, ff, hl, en, as, ik, csw, U, k, hE, X); #c1(38231) c2(NP_004565) c3(septin-4 isoform 1) c4(147708) c5(147709, 147710, 147711, 147712, 147713, 147714, 147715, 147716, 147717, 147718, 147719, 147720, 147721, 147722, 147723, 147724, 147725, 147726, 147727, 147728, 147729, 147730, 147731, 147732, 147733, 147734, 147735, 147736, 147737, 147738, 147739, 147740, 147741, 147742, 147743, 147744, 147745, 147746, 147747, 147748, 147749, 147750, 147751, 147752, 147753, 147754, 147755, 147756, 147757, 147758, 147759, 147760, 147761, 147762, 147763, 147764, 147765, 147766, 147767, 147768, 147769, 147770, 147771, 147772, 147773, 147774, 147775, 147776, 147777, 147778, 147779, 147780, 147781, 147782, 147783, 147784, 147785, 147786, 147787, 147788, 147789, 147790, 147791, 147792, 147793, 147794, 147795, 147796, 147797, 147798, 147799, 147800, 147801, 147802, 147803, 147804) c6(147805, 147806) c7(T, ad, XX, x, aT, K, ae, azh, ago, d, B, aW, ff, hl, en, as, iK, csw, U, k, hE, X); #c1(38231) c2(NP_536340) c3(septin-4 isoform 2) c4(231242) c5(231243, 231244, 231245, 231246, 231247, 231248) c6( ) c7(T, ad, XX, x, aT, K, ae, azh, ago, d, B, aW, ff, hl, en, as, ik, csw, U, k, hE, X); #c1(38231) c2(XP_006722012) c3(septin-4 isoform X3) c4( ) c5(294540, 294541, 294542, 294543, 294544, 294545, 294546, 294547, 294548, 294549, 294550, 294551) c6( ) c7(T, ad, XX, x, aT, K, ae, azh, ago, d, B, aW, ff, hl, en, as, ik, csw, U, k, hE, X); #c1(38231) c2(XP_006722015) c3(septin-4 isoform X6) c4(294552) c5( ) c6( ) c7(T, ad, XX, x, aT, K, ae, azh, ago, d, B, aW, ff, hl, en, as, ik, csw, U, k, hE, X); #c1(38231) c2(XP_011523212) c3(septin-4 isoform X2) c4(317731, 317732) c5( ) c6( ) c7(T, ad, XX, x, aT, K, ae, azh, ago, d, B, aW, ff, hl, en, as, ik, csw, U, k, hE, X); #c1(38231) c2(XP_011523214) c3(septin-4 isoform X1) c4( ) c5(317733, 317734, 317735, 317736, 317737) c6( ) c7(T, ad, XX, x, aT, K, ae, azh, ago, d, B, aW, ff, hl, en, as, iK, csw, U, k, hE, X); #c1(38596) c2(NP_001009939) c3(septin-5 isoform 2) c4(23757) c5(23758, 23759, 23760, 23761, 23762, 23763, 23764, 23765, 23766, 23767, 23768) c6( ) c7(bL, ck, x, bgk, iT, RX, bpp, U, k, bt); #c1(38596) c2(NP_002679) c3(septin-5 isoform 1) c4(130869, 130870, 130871, 130872) c5(130873, 130874, 130875, 130876, 130877, 130878, 130879, 130880, 130881, 130882, 130883, 130884, 130885, 130886, 130887, 130888, 130889, 130890, 130891, 130892, 130893, 130894, 130895) c6( ) c7(bL, ck, x, bgk, iT, RX, bpp, U, k, bt); #c1(38961) c2(NP_055944) c3(septin-6 isoform B) c4(184588) c5(184589, 184590, 184591, 184592, 184593, 184594) c6( ) c7(ck, dz, iT, bt); #c1(38961) c2(NP_665801) c3(septin-6 isoform D) c4(241691) c5(241692, 241693, 241694, 241695, 241696, 241697) c6( ) c7(ck, dz, iT, bt); #c1(38961) c2(XP_005262457) c3(septin-6 isoform X3) c4( ) c5(277963) c6( ) c7(ck, dz, iT, bt); #c1(38961) c2(XP_006724811) c3(septin-6 isoform X5) c4(296400) c5(296401, 296402, 296403, 296404, 296405, 296406) c6( ) c7(ck, dz, iT, bt); #c1(38961) c2(XP_006724813) c3(septin-6 isoform X1) c4(296407, 296408, 296409) c5(296410, 296411, 296412, 296413, 296414, 296415, 296416, 296417, 296418, 296419, 296420, 296421, 296422, 296423, 296424, 296425, 296426, 296427, 296428, 296429, 296430, 296431, 296432, 296433, 296434, 296435, 296436, 296437, 296438) c6( ) c7(ck, dz, iT, bt); #c1(38961) c2(XP_011529619) c3(septin-6 isoform X2) c4(325154) c5( ) c6( ) c7(ck, dz, iT, bt); #c1(38961) c2(XP_011529620) c3(septin-6 isoform X4) c4( ) c5(325155, 325156) c6( ) c7(ck, dz, iT, bt); #c1(39326) c2(NP_001011553) c3(septin-7 isoform 2) c4(24618, 24619, 24620) c5(24621, 24622, 24623, 24624, 24625, 24626, 24627, 24628, 24629, 24630, 24631, 24632, 24633, 24634, 24635, 24636, 24637, 24638, 24639, 24640, 24641, 24642, 24643, 24644, 24645, 24646, 24647) c6( ) c7(h, eA, d, K, as); #c1(39326) c2(NP_001229885) c3(septin-7 isoform 3) c4(85490) c5(85491, 85492, 85493, 85494, 85495, 85496, 85497, 85498, 85499, 85500, 85501, 85502, 85503, 85504, 85505, 85506, 85507, 85508, 85509, 85510, 85511, 85512) c6( ) c7(h, eA, d, K, as); #c1(39326) c2(NP_001779) c3(septin-7 isoform 1) c4(121033, 121034, (21035) c5(121036, 121037, 121038, 121039, 121040, 121041, 121042) c6( ) c7(h, eA, d, K, as); #c1(39326) c2(XP_006715869) c3(septin-7 isoform X6) c4(289882) c5( ) c6( ) c7(h, eA, d, K, as); #c1(39326) c2(XP_011513962) c3(septin-7 isoform X5) c4( ) c5(306405, 306406, 306407, 306408, 306409, 306410) c6( ) c7(h, eA, d, K, as); #c1(39326) c2(XP_011513964) c3(septin-7 isoform X8) c4(306411) c5( ) c6( ) c7(h, eA, d, K, as); #c1(40057) c2(NP_001106963) c3(septin-9 isoform a) c4(42685, 42686) c5(42687, 42688, 42689, 42690, 42691, 42692, 42693) c6( ) c7(cCO, T, qx, bB, amj, ci, fu, bX, q, ad, aE, dV, aT, K, ae, pl, s, NE, aW, r, aot, t, cCQ, ik, bw, ck, iT, csw, dB, bt, cCP); #c1(40057) c2(NP_001106965) c3(septin-9 isoform b) c4(42694) c5(42695, 42696, 42697, 42698, 42699, 42700, 42701, 42702) c6( ) c7(cCO, T, qx, bB, amj, ci, fu, bX, q, ad, aE, dV, aT, K, ae, pl, s, NE, aW, r, aot, t, cCQ, ik, bw, ck, iT, csw, dB, bt, cCP); #c1(40057) c2(NP_001106966) c3(septin-9 isoform e) c4( ) c5(42703, 42704, 42705, 42706, 42707, 42708, 42709, 42710, 42711, 42712, 42713, 42714, 42715, 42716) c6( ) c7(cCO, T, qx, bB, amj, ci, fu, bX, q, ad, aE, dV, aT, K, ae, pl, s, NE, aW, r, aot, t, cCQ, ik, bw, ck, iT, csw, dB, bt, cCP); #c1(40057) c2(NP_001106967) c3(septin-9 isoform d) c4(42717) c5(42718, 42719, 42720, 42721, 42722, 42723) c6( ) c7(cCO, T, qx, bB, amj, ci, fu, bX, q, ad, aE, dV, aT, K, ae, pl, s, NE, aW, r, aot, t, cCQ, ik, bw, ck, iT, csw, dB, bt, cCP); #c1(40057) c2(NP_001280625) c3(septin-9 isoform h) c4(111543) c5(111544, 111545, 111546, 111547, 111548, 111549, 111550, 111551) c6( ) c7(cCO, T, qx, bB, amj, ci, fu, bX, q, ad, aE, dV, aT, K, ae, pl, s, NE, aW, r, aot, t, cCQ, ik, bw, ck, iT, csw, dB, bt, cCP); #c1(40057) c2(NP_001280626) c3(septin-9 isoform f) c4( ) c5(111552, 111553, 111554, 111555, 111556, 111557, 111558, 111559, 111560, 111561, 111562, 111563, 111564) c6( ) c7(cCO, T, qx, bB, amj, ci, fu, bX, q, ad, aE, dV, aT, K, ae, pl, s, NE, aW, r, aot, t, cCQ, ik, bw, ck, iT, csw, dB, bt, cCP); #c1(40057) c2(NP_006631) c3(septin-9 isoform c) c4(166737) c5(166738, 166739, 166740, 166741, 166742, 166743, 166744, 166745, 166746) c6( ) c7(cCO, T, qx, bB, amj, ci, fu, bX, q, ad, aE, dV, aT, K, ae, pl, s, NE, aW, r, aot, t, cCQ, ik, bw, ck, iT, csw, dB, bt, cCP); #c1(40057) c2(XP_011522507) c3 (septin-9 isoform X4) c4( ) c5(317134) c6( ) c7(cCO, T, qx, bB, amj, ci, fu, bX, q, ad, aE, dV, aT, K, ae, pl, s, NE, aW, r, aot, t, cCQ, ik, bw, ck, iT, csw, dB, bt, cCP); #c1(40057) c2(XP_011522508) c3(septin-9 isoform X5) c4(317135, 317136) c5( ) c6( ) c7(cCO, T, qx, bB, amj, ci, fu, bX, q, ad, aE, dV, aT, K, ae, pl, s, NE, aW, r, aot, t, cCQ, ik, bw, ck, iT, csw, dB, bt, cCP); #c1(40422) c2(NP_653311) c3(septin-10 isoform 1) c4(239550, 239551, 239552) c5(239553, 239554, 239555, 239556, 239557, 239558) c6( ) c7(T, ci); #c1(40422) c2(NP_848699) c3(septin-10 isoform 2) c4(254191) c5(254192, 254193, 254194, 254195, 254196, 254197) c6( ) c7(T, ci); #c1(40422) c2(XP_006712380) c3(septin-10 isoform X5) c4(286629) c5(286630, 286631, 286632, 286633, 286634, 286635, 286636, 286637, 286638, 286639, 286640) c6( ) c7(T, ci); #c1(40422) c2(XP_011508999) c3(septin-10 isoform X1) c4(299585) c5(299586, 299587, 299588, 299589, 299590, 299591) c6( ) c7(T, ci); #c1(40422) c2(XP_011509003) c3(septin-10 isoform X6) c4( ) c5(299592, 299593, 299594, 299595, 299596, 299597) c6( ) c7(T, ci); #c1(40422) c2(XP_011509005) c3(septin-10 isoform X8) c4( ) c5(299598) c6( ) c7(T, ci); #c1(40422) c2(XP_011509006) c3(septin-10 isoform X9) c4(299599) c5(299600, 299601, 299602, 299603, 299604, 299605) c6( ) c7(T, ci); #c1(40422) c2(XP_011509007) c3(septin-10 isoform X10) c4(299606) c5(299607, 299608, 299609, 299610, 299611, 299612) c6( ) c7(T, ci); #c1(40787) c2(NP_060713) c3(septin-11) c4(197388, 197389) c5(197390, 197391, 197392, 197393, 197394, 197395, 197396, 197397, 197398, 197399, 197400, 197401) c6( ) c7(ck, csw, WY, aaE, bK, bt); #c1(41153) c2(NP_001147930) c3(septin-12 isoform 1) c4(60098) c5(60099, 60100, 60101, 60102, 60103, 60104) c6( ) c7(cCR, gU); #c1(41153) c2(NP_653206) c3(septin-12 isoform 2) c4(239064) c5(239065, 239066, 239067, 239068, 239069, 239070) c6( ) c7(cCR, gU); #c1(41883) c2(NP_997249) c3(septin-14) c4(265318, 265319) c5(265320, 265321, 265322, 265323, 265324, 265325, 265326, 265327, 265328, 265329, 265330, 265331) c6( ) c7(d); #c1(42248) c2(NP_004252) c3(15 kDa selenoprotein isoform 1 precursor) c4(144609, 144610) c5(144611, 144612, 144613, 144614, 144615, 144616, 144617) c6( ) c7(av, T, eM, bE, eL, bB, at, dL, bz, ae, B, ad); #c1(SEPHS1) c2(NP_001182531) c3(selenide, water dikinase 1 isoform 2) c4( ) c5(74992, 74993, 74994, 74995, 74996) c6( ) c7(gl, fq, iu, K); #c1(SEPHS1) c2(NP_001182533) c3(selenide, water dikinase 1 isoform 3) c4(74997) c5(74998, 74999, 75000, 75001, 75002, 75003, 75004, 75005, 75006, 75007, 75008, 75009, 75010, 75011, 75012, 75013, 75014, 75015, 75016, 75017, 75018, 75019) c6( ) c7(gl, fq, iu, K); #c1(SEPHS1) c2(NP_036379) c3(selenide, water dikinase 1 isoform 1) c4(173379, 173380, 173381, 173382, 173383) c5(173384, 173385, 173386, 173387, 173388, 173389, 173390, 173391, 173392, 173393, 173394, 173395, 173396) c6( ) c7(gl, fq, iu, K); #c1(SEPHS2) c2(NP_036380) c3(selenide, water dikinase 2) c4(173397, 173398, 173399, 173400) c5(173401, 173402, 173403, 173404, 173405, 173406, 173407, 173408, 173409, 173410, 173411, 173412, 173413, 173414, 173415, 173416, 173417, 173418, 173419, 173420, 173421, 173422, 173423, 173424) c6( ) c7(iu); #c1(SEPP1) c2(NP_001087195) c3(selenoprotein P isoform 2) c4(38248, 38249, 38250) c5(38251, 38252, 38253, 38254, 38255, 38256) c6( ) c7(bL, oq, T, gU, dD, pf, ea, bmk, aw, bB, aj, j, ci, eq, zP, q, aR, J, ad, dP, av, GL, HC, aT, dL, K, ae, zQ, d, s, agT, aW, eZ, r, fe, aq, LB, ac, eM, nB, bE, U, at, im, xg, B); #c1(SEPP1) c2(NP_005401) c3(selenoprotein P isoform 1 precursor) c4( ) c5(155733, 155734, 155735, 155736, 155737, 155738, 155739, 155740, 155741, 155742, 155743, 155744, 155745, 155746, 155747, 155748, 155749, 155750, 155751, 155752, 155753, 155754) c6( ) c7(bL, oq, T, gU, dD, pf, ea, bmk, aw, bB, aj, j, ci, eq, zP, q, aR, J, ad, dP, av, GL, HC, aT, dL, K, ae, zQ, d, s, agT, aW, eZ, r, fe, aq, LB, ac, eM, nB, bE, U, at, im, xg, B); #c1(SEPSECS) c2(NP_058651) c3(O-phosphoseryl-tRNA(Sec) selenium transferase) c4(193020) c5(193021, 193022, 193023, 193024, 193025, 193026) c6( ) c7(qp, cCS, bh); #c1(SEPSECS) c2(XP_005248225) c3(O-phosphoseryl-tRNA(Sec) selenium transferase isoform X4) c4(268272) c5(268273, 268274, 268275, 268276, 268277, 268278) c6( ) c7(qp, cCS, bh); #c1(SEPW1) c2(NP_003000) c3(selenoprotein W) c4(134311, 134312) c5(134313, 134314, 134315, 134316, 134317, 134318, 134319, 134320, 134321, 134322, 134323, 134324) c6( ) c7(blr, ha, fd); #c1(SERAC1) c2(NP_116250) c3(protein SERAC1) c4(225956, 225957) c5(225958, 225959, 225960, 225961, 225962, 225963, 225964, 225965, 225966, 225967, 225968, 225969, 225970, 225971, 225972, 225973, 225974, 225975, 225976, 225977, 225978, 225979) c6(225980, 225981, 225982, 225983, 225984, 225985) c7(alD, nU, cmH, aU, cCT, cd); #c1(SERAC1) c2(XP_011534499) c3(protein SERAC1 isoform X2) c4(330957) c5( ) c6( ) c7(alD, nu, cmH, aU, cCT, cd); #c1(SERAC1) c2(XP_011534500) c3(protein SERAC1 isoform X3) c4( ) c5(330958, 330959, 330960, 330961, 330962, 330963, 330964, 330965) c6(330966, 330967) c7(alD, nU, cmH, aU, cCT, cd); #c1(SERF1A) c2(NP_068802) c3(small EDRK-rich factor 1 isoform 1) c4(210617, 210618, 210619) c5(210620, 210621, 210622, 210623, 210624, 210625, 210626, 210627, 210628, 210629, 210630, 210631, 210632, 210633, 210634, 210635, 210636, 210637, 210638, 210639, 210640, 210641, 210642) c6( ) c7(kf, arX, bSl, v, apl); #c1(SERF1A) c2(NP_075257) c3(small EDRK-rich factor 1 isoform 2) c4(213744, 213745, 213746) c5(213747, 213748, 213749, 213750, 213751, 213752, 213753, 213754, 213755, 213756, 213757, 213758, 213759, 213760, 213761, 213762, 213763, 213764, 213765, 213766, 213767) c6( ) c7(kf, arX, bSl, v, apl); #c1(SERGEF) c2(NP_036271) c3(secretion-regulating guanine nucleotide exchange factor) c4(172747, 172748, 172749) c5(172750, 172751, 172752, 172753, 172754, 172755, 172756, 172757, 172758, 172759, 172760, 172761, 172762, 172763, 172764, 172765, 172766, 172767) c6( ) c7(pN); #c1(SERINC1) c2(NP_065806) c3(serine incorporator 1 precursor) c4(205631) c5(205632, 205633, 205634, 205635, 205636, 205637, 205638, 205639, 205640, 205641) c6( ) c7(av, eM, bu); #c1(SERINC3) c2(NP_006802) c3(serine incorporator 3 precursor) c4(168206, 168207, 168208, 168209) c5(168210, 168211, 168212, 168213, 168214, 168215, 168216, 168217, 168218, 168219, 168220, 168221, 168222, 168223, 168224, 168225, 168226, 168227, 168228, 168229, 168230, 168231, 168232, 168233, 168234, 168235, 168236, 168237) c6( ) c7(ad, bu); #c1(SERINC5) c2(NP_001167542) c3(serine incorporator 5 isoform 3) c4(69057) c5(69058, 69059, 69060, 69061, 69062, 69063) c6( ) c7(aon); #c1(SERINC5) c2(NP_001167543) c3(serine incorporator 5 isoform 1) c4(69064) c5(69065, 69066, 69067, 69068, 69069, 69070) c6( ) c7(aon); #c1(SERINC5) c2(NP_840060) c3(serine incorporator 5 isoform 2) c4(253614, 253615) c5(253616, 253617, 253618, 253619, 253620, 253621, 253622, 253623, 253624, 253625, 253626, 253627) c6( ) c7(aon); #c1(SERP1) c2(NP_055260) c3(stress-associated endoplasmic reticulum protein 1) c4(180119) c5(180120, 180121, 180122, 180123, 180124, 180125, 180126, 180127, 180128, 180129) c6( ) c7(mf, K); #c1(SERP2) c2(NP_001010897) c3(stress-associated endoplasmic reticulum protein 2) c4(24029) c5(24030, 24031, 24032, 24033, 24034, 24035) c6( ) c7(ck, dh); #c1(SERPINA1) c2(NP_001121179) c3(alpha-1-antitrypsin precursor) c4(45338, 45339, 45340, 45341, 45342, 45343, 45344, 45345, 45346, 45347, 45348) c5(45349, 45350, 45351, 45352, 45353, 45354, 45355, 45356, 45357, 45358, 45359, 45360, 45361, 45362, 45363, 45364, 45365, 45366, 45367, 45368, 45369, 45370, 45371, 45372, 45373, 45374, 45375, 45376, 45377, 45378, 45379, 45380, 45381, 45382, 45383, 45384, 45385, 45386, 45387, 45388, 45389, 45390, 45391, 45392, 45393, 45394, 45395, 45396, 45397, 45398, 45399, 45400, 45401, 45402, 45403, 45404, 45405, 45406, 45407, 45408, 45409, 45410, 45411, 45412, 45413, 45414, 45415, 45416, 45417, 45418, 45419, 45420, 45421, 45422, 45423, 45424, 45425, 45426, 45427, 45428, 45429, 45430, 45431, 45432, 45433, 45434, 45435, 45436, 45437, 45438, 45439, 45440, 45441, 45442, 45443, 45444, 45445, 45446, 45447, 45448, 45449, 45450, 45451, 45452, 45453, 45454, 45455, 45456, 45457, 45458, 45459, 45460, 45461, 45462, 45463, 45464, 45465, 45466, 45467, 45468, 45469, 45470, 45471, 45472, 45473, 45474, 45475, 45476, 45477, 45478, 45479, 45480, 45481, 45482, 45483, 45484, 45485, 45486, 45487, 45488, 45489, 45490, 45491, 45492, 45493, 45494, 45495, 45496, 45497, 45498, 45499, 45500, 45501, 45502, 45503, 45504, 45505, 45506, 45507, 45508, 45509, 45510, 45511, 45512, 45513, 45514, 45515, 45516, 45517, 45518, 45519, 45520, 45521, 45522, 45523, 45524, 45525, 45526, 45527, 45528, 45529, 45530, 45531, 45532, 45533, 45534, 45535, 45536, 45537, 45538, 45539, 45540, 45541, 45542, 45543, 45544, 45545, 45546, 45547, 45548, 45549, 45550, 45551, 45552, 45553, 45554, 45555, 45556, 45557, 45558, 45559, 45560, 45561, 45562, 45563, 45564, 45565, 45566, 45567, 45568, 45569, 45570, 45571, 45572, 45573, 45574, 45575, 45576, 45577, 45578, 45579, 45580, 45581, 45582, 45583, 45584, 45585, 45586, 45587, 45588, 45589, 45590, 45591, 45592, 45593, 45594, 45595, 45596, 45597, 45598, 45599, 45600, 45601, 45602, 45603, 45604, 45605, 45606, 45607, 45608, 45609, 45610, 45611, 45612, 45613, 45614, 45615, 45616, 45617, 45618, 45619, 45620, 45621, 45622, 45623, 45624, 45625, 45626, 45627, 45628, 45629, 45630, 45631, 45632, 45633) c6(45634, 45635, 45636, 45637, 45638, 45639, 45640, 45641, 45642, 45643, 45644, 45645, 45646, 45647, 45648, 45649, 45650, 45651, 45652, 45653, 45654, 45655, 45656, 45657, 45658, 45659, 45660, 45661, 45662) c7(bM, T, Fj, FR, lP, ea, e, al, ame, ci, fw, sD, Tz, xE, aE, dv, cCZ, Ex, us, K, wt, H, bc, N, aa, cCX, fc, hl, aDn, kt, bBH, g, hJ, ve, DL, y, aBr, eM, lq, U, bjL, Uw, fy, dD, cCU, lo, yG, aw, aj, qN, qW, ai, jw, ke, auv, b, zw, azj, cU, h, eV, rk, A, mR, ap, aq, jT, akv, cDa, afj, hq, Wt, vU, ar, W, cA, bpC, cCW, bo, aic, JM, at, uz, bm, eo, B, eR, jp, dk, oe, bqv, fG, jx, aA, cz, fF, ew, v, j, ex, V, q, J, mj, qp, aT, yu, bK, yb, caR, af, ak, bf, iU, oV, bg, r, afq, lo, bQ, f, kC, pb, FT, axS, pa, cCV, alC, nM, CP, DW, fD, UF, ao, aya, vi, hS, am, qb, uG, jh, dG, Jz, eq, iG, afD, ad, kL, hX, qC, av, F, Db, oz, le, lC, lQ, lz, NP, BO, aus, cr, yy, dq, hv, aF, hp, Fq, iV, iR, bz, dr, aYt, TA, gk, mO, nb, axs, cCY, eB, dX, aS, cbR, i, hE, ra, sH); #c1(SERPINA10) c2(NP_001094077) c3(protein Z-dependent protease inhibitor precursor) c4( ) c5(40617, 40618, 40619, 40620, 40621, 40622, 40623, 40624, 40625, 40626, 40627, 40628, 40629, 40630, 40631, 40632, 40633) c6( ) c7(ag, jv, aw); #c1(SERPINA10) c2(XP_006725226) c3(protein Z-dependent protease inhibitor isoform X1) c4(296719, 296720) c5(296721, 296722, 296723, 296724, 296725, 296726, 296727, 296728, 296729, 296730, 296731) c6( ) c7(ag, jv, aw); #c1(SERPINA11) c2(NP_001073920) c3(serpin A11 precursor) c4(36542) c5(36543, 36544, 36545, 36546, 36547, 36548, 36549, 36550) c6(36551, 36552) c7(iR, aq); #c1(SERPINA12) c2(NP_001291390) c3(serpin A12 precursor) c4(115840) c5(115841, 115842, 115843, 115844, 115845, 115846, 115847, 115848, 115849, 115850, 115851, 115852, 115853, 115854) c6(115855, 115856, 115857, 115858) c7(bL, kl, j, K, v); #c1(SERPINA3) c2(NP_001076) c3(alpha-1-antichymotrypsin precursor) c4(37583, 37584, 37585, 37586) c5( ) c6( ) c7(bL, bM, G, dt, T, am, eR, hV, oe, lo, jN, l, cz, fF, GC, aj, aw, j, ct, ci, PJ, eg, cDb, qN, qW, fK, J, cd, ad, qH, eA, AW, Hx, av, sO, crU, gd, M, aT, us, dV, iv, bK, an, H, ae, af, N, hm, aa, ak, A, cH, aW, sp, hl, ap, hv, aq, aUO, aaO, t, alj, KM, ve, wA, iO, pu, W, c, ac, Kv, AV, z, U, vy, at, dX, aS, tr, du, B, abh, L); #c1(SERPINA4) c2(NP_001275961) c3(kallistatin isoform 1) c4( ) c5(107544, 107545, 107546) c6( ) c7(ai, wG, LC, aw, nd, X, RJ, aT, v, nA, UF, he, btr, cE, B, yn); #c1(SERPINA4) c2(NP_006206) c3(kallistatin isoform 2 precursor) c4(162837) c5(162838, 162839, 162840, 162841, 162842, 162843, 162844, 162845, 162846, 162847, 162848) c6( ) c7(ai, wG, LC, aw, nd, X, RJ, aT, v, nA, UF, he, btr, cE, B, yn); #c1(SERPINA5) c2(NP_000615) c3(plasma serine protease inhibitor preproprotein) c4(13802, 13803, 13804, 13805, 13806, 13807, 13808, 13809, 13810, 13811, 13812) c5(13813, 13814, 13815, 13816, 13817, 13818, 13819, 13820, 13821, 13822, 13823, 13824, 13825, 13826, 13827, 13828, 13829, 13830, 13831, 13832, 13833, 13834, 13835, 13836, 13837, 13838, 13839, 13840, 13841, 13842, 13843, 13844, 13845, 13846, 13847, 13848, 13849, 13850, 13851, 13852, 13853, 13854, 13855, 13856, 13857, 13858, 13859, 13860, 13861, 13862, 13863, 13864, 13865, 13866, 13867, 13868, 13869, 13870, 13871, 13872, 13873, 13874, 13875, 13876, 13877, 13878, 13879, 13880, 13881, 13882, 13883, 13884, 13885, 13886, 13887, 13888, 13889, 13890, 13891, 13892, 13893, 13894, 13895, 13896, 13897, 13898, 13899, 13900, 13901, 13902, 13903, 13904, 13905, 13906, 13907, 13908, 13909, 13910, 13911, 13912, 13913, 13914, 13915) c6(13916, 13917, 13918, 13919, 13920, 13921, 13922, 13923, 13924, 13925, 13926, 13927, 13928, 13929, 13930, 13931, 13932, 13933, 13934, 13935, 13936, 13937, 13938, 13939, 13940) c7(T, am, gU, dA, iW, aw, ex, jh, fd, fr, ci, zn, aE, aT, vH, io, eV, xa, N, aW, hQ, nY, aq, ve, t, iK, es, ag, bm, cj, m, lC, B); #c1(SERPINA6) c2(NP_001747) c3(corticosteroid-binding globulin precursor) c4(120720, 120721) c5(120722, 120723, 120724, 120725, 120726, 120727, 120728, 120729, 120730, 120731, 120732, 120733) c6( ) c7(T, am, iW, cz, cL, v, wy, cDc, aVM, ctp, jy, hn, K, aVL, aq, cL, ve, t, dr, ck, il, sn, hr, m, ei, B); #c1(SERPINA7) c2(NP_000345) c3(thyroxine-binding globulin precursor) c4(7599) c5(7600, 7601, 7602, 7603, 7604, 7605, 7606, 7607, 7608, 7609, 7610, 7611, 7612, 7613) c6(7614, 7615, 7616, 7617) c7(qC, iO, cDe, mO, ob, aus, RK, cDd, bfA, hE, hr, bzb, aq, gT, Ze, B, Rf); #c1(SERPINA9) c2(NP_001035983) c3(serpin A9 isoform B) c4(33624) c5(33625, 33626, 33627, 33628, 33629, 33630, 33631, 33632) c6(33633, 33634) c7(fM, ac, e, aw, jh, aq, j, iR, pk, eu); #c1(SERPINA9) c2(NP_001271204) c3(serpin A9 isoform C) c4(103590, 103591) c5(103592, 103593, 103594, 103595, 103596, 103597, 103598, 103599, 103600) c6(103601, 103602) c7(fM, ac, e, aw, jh, aq, j, R, pk, eu); #c1(SERPINA9) c2(NP_001271205) c3(serpin A9 isoform D) c4( ) c5(103603, 103604, 103605, 103606, 103607, 103608, 103609) c6(103610, 103611) c7(fM, ac, e, aw, jh, aq, j, iR, pk, eu); #c1(SERPINA9) c2(NP_783866) c3(serpin A9 isoform A) c4(251896) c5(251897, 251898, 251899, 251900, 251901, 251902, 251903, 251904, 251905) c6(251906, 251907) c7(fM, ac, e, aw, jh, aq, j, iR, pk, eu); #c1(SERPINA9) c2(XP_011535017) c3(serpin A9 isoform X1) c4(331697, 331698) c5(331699, 331700, 331701, 331702, 331703, 331704, 331705) c6(331706, 331707) c7(fM, ac, e, aw, jh, aq, j, iR, pk, eu); #c1(SERPINA9) c2(XP_011535019) c3(serpin A9 isoform X2) c4( ) c5(331708, 331709, 331710, 331711, 331712, 331713, 331714, 331715) c6(331716, 331717) c7(fM, ac, e, aw, jh, aq, j, iR, pk, eu); #c1(SERPINB1) c2(NP_109591) c3(leukocyte elastase inhibitor) c4(219723, 219724, 219725) c5(219726, 219727, 219728, 219729, 219730, 219731, 219732, 219733, 219734, 219735, 219736, 219737, 219738, 219739, 219740, 219741, 219742) c6( ) c7(dD, vj, fF, lo, uJ, eq, aa); #c1(SERPINB1) c2(XP_011512637) c3(leukocyte elastase inhibitor isoform X2) c4( ) c5(303600, 303601, 303602, 303603, 303604, 303605, 303606) c6( ) c7(dD, vj, fF, lo, uJ, eq, aa); #c1(SERPINB10) c2(NP_005015) c3(serpin BIO) c4(152179) c5(152180, 152181, 152182, 152183, 152184, 152185) c6( ) c7(cqK, ad); #c1(SERPINB13) c2(NP_036529) c3(serpin B13) c4(174250, 174251) c5(174252, 174253, 174254, 174255, 174256, 174257, 174258, 174259, 174260, 174261, 174262, 174263) c6( ) c7(ff, en); #c1(SERPINB13) c2(XP_005266764) c3(serpin B13 isoform X3) c4( ) c5(280318, 280319, 280320, 280321, 280322, 280323) c6( ) c7(ff, en); #c1(SERPINB2) c2(NP_001137290) c3(plasminogen activator inhibitor 2) c4(55259, 55260, 55261, 55262) c5(55263, 55264, 55265, 55266, 55267, 55268, 55269, 55270, 55271, 55272, 55273, 55274, 55275, 55276, 55277, 55278, 55279, 55280, 55281, 55282, 55283, 55284, 55285, 55286, 55287, 55288, 55289, 55290) c6( ) c7(bL, bM, T, aCA, akd, QO, wu, aZ, aD, aw, Ju, iW, aA, ez, OR, aj, Cg, j, v, ape, bX, fd, V, q, J, ad, ft, lg, qp, aT, h, dz, K, wm, dQ, adx, ae, dl, Df, eG, OV, lR, aW, Y, sq, az, bar, eZ, r, ff, aF, aB, uF, en, Sm, g, t, iK, bz, DF, ck, cT, ac, eM, bLZ, iT, Fi, ag, cof, vy, hL, wq, uJ, zv, m, EY, ei, it, B); #c1(SERPINB3) c2(NP_008850) c3(serpin B3) c4(169110) c5(169111, 169112, 169113, 169114, 169115, 169116) c6( ) c7(bM, T, fG, ht, al, ccx, n, ci, hX, ccw, aT, K, aW, ff, hl, cr, eZ, aq, en, g, eE, t, ik, bz, iR, LB, iO, gk, eM, m, hE, it, B, vi); #c1(SERPINB4) c2(NP_778206) c3(serpin B4 isoform 2) c4(251406) c5(251407, 251408, 251409, 251410, 251411, 251412) c6( ) c7(bM, T, yk, if, ccx, n, qN, hX, ccw, hw, aT, K, aW, ff, hl, cr, aq, en, g, eE, ik, bz, eM, hk, adg, hE, rA, B); #c1(SERPINB4) c2(XP_011524440) c3(serpin B4 isoform X1) c4(319025) c5(319026, 319027, 319028, 319029, 319030, 319031) c6( ) c7(bM, T, yk, if, ccx, n, qN, hX, ccw, hw, aT, K, aW, ff, hl, cr, aq, en, g, eE, iK, bz, eM, hk, adg, hE, rA, B); #c1(SERPINB5) c2(NP_002630) c3(serpin 85) c4(130476, 130477) c5(130478, 130479, 130480, 130481, 130482, 130483, 130484, 130485, 130486, (30487, 130488, 130489) c6( ) c7(T, aC, aA, bB, ci, AO, q, ad, av, hd, aE, aT, dL, ae, Df, eL, s, qt, aW, Y, ff, r, kp, aF, en, g, t, bz, eM, nB, bE, Qu, hL, m, X); #c1(SERPINB6) c2(NP_001182220) c3(serpin B6 isoform b) c4(74719, 74720) c5(74721, 74722, 74723, 74724) c6( ) c7(bM, T, Ke, jx, bB, asr, q, eW, F, cDf, afn, aT, h, K, avR, aW, r, tv, aq, bHg, tu, Zb, eK, avT, cT, rR, ac, avS, bE, asq, mG, B, Tc); #c1(SERPINB6) c2(NP_001258751) c3(serpin B6 isoform c) c4( ) c5(96502, 96503) c6( ) c7(bM, T, Ke, jx, bB, asr, q, eW, F, cDf, afn, aT, h, K, avR, aW, r, tv, aq, bHg, tu, Zb, ek, avT, cT, rR, ac, avS, bE, asq, mG, B, Tc); #c1(SERPINB6) c2(NP_001258752) c3(serpin B6 isoform d) c4(96504) c5(96505) c6( ) c7(bM, T, Ke, jx, bB, asr, q, eW, F, cDf, afn, aT, h, K, avR, aW, r, tv, aq, bHg, tu, Zb, ek, avT, cT, rR, ac, avS, bE, asq, mG, B, Tc); #c1(SERPINB6) c2(NP_001284628) c3(serpin B6 isoform a) c4( ) c5(112173, 112174, 112175, 112176, 112177, 112178, 112179, 112180, 112181, 112182, 112183, 112184, 112185, 112186, 112187, 112188, 112189, 112190, 112191, 112192, 112193, 112194, 112195, 112196, 112197) c6( ) c7(bM, T, Ke, jx, bB, asr, q, eW, F, cDf, afn, aT, h, K, avR, aW, r, tv, aq, bHg, tu, Zb, ek, avT, cT, rR, ac, avS, bE, asq, mG, B, Tc); #c1(SERPINB6) c2(XP_011512978) c3(serpin 86 isoform X3) c4( ) c5(304434, 304435, 304436, 304437, 304438, 304439) c6( ) c7(bM, T, Ke, jx, bB, asr, q, eW, F, cDf, afn, aT, h, K, avR, aW, r, tv, aq, bHg, tu, Zb, eK, avT, cT, rR, ac, avS, bE, asq, mG, B, Tc); #c1(SERPINB7) c2(NP_001248760) c3(serpin 87 isoform 2) c4(94009) c5(94010, 94011, 94012, 94013, 94014, 94015) c6( ) c7(eR, cDg, by, aj, ey, uE, t); #c1(SERPINB7) c2(XP_011524538) c3(serpin B7 isoform X1) c4(319113, 319114, 319115) c5(319116, 319117, 319118, 319119, 319120, 319121, 319122, 319123, 319124, 319125, 319126, 319127, 319128, 319129, 319130, 319131, 319132, 319133, 319134, 319135, 319136, 319137, 319138, 319139, 319140, 319141, 319142, 319143, 319144, 319145) c6( ) c7(eR, cDg, by, aj, ey, uE, t); #c1(SERPINB8) c2(NP_001027018) c3(serpin 88 isoform b) c4(30069, 30070) c5(30071, 30072, 30073, 30074, 30075, 30076, 30077, 30078, 30079, 30080, 30081, 30082, 30083, 30084, 30085, 30086, 30087, 30088) c6( ) c7(ac, Jr, e, aw, cr, wR, j, B, eu); #c1(SERPINB8) c2(NP_001263419) c3(serpin B8 isoform c) c4( ) c5(97284, 97285, 97286, 97287, 97288) c6( ) c7(ac, Jr, e, aw, cr, wR, j, B, eu); #c1(SERPINB8) c2(NP_942130) c3(serpin 88 isoform a) c4(260481, 260482) c5(260483, 260484, 260485, 260486, 260487, 260488, 260489, 260490, 260491, 260492, 260493) c6( ) c7(ac, Jr, e, aw, cr, wR, j, B, eu); #c1(SERPINB8) c2(XP_011524327) c3(serpin B8 isoform X1) c4(319001) c5( ) c6( ) c7(ac, Jr, e, aw, cr, wR, j, B, eu); #c1(SERPINB9) c2(XP_005249241) c3(serpin B9 isoform X1) c4(269141) c5(269142, 269143, 269144, 269145, 269146, 269147) c6( ) c7(Nl, lo, Yn, Y, uD); #c1(SERPINB9) c2(XP_011512980) c3(serpin B9 isoform X2) c4( ) c5(304440) c6( ) c7(Nl, lo, Yn, Y, uD); #c1(SERPINC1) c2(NP_000479) c3(antithrombin-Ill precursor) c4(9485, 9486) c5(9487, 9488, 9489, 9490, 9491, 9492, 9493, 9494, 9495, 9496, 9497, 9498, 9499, 9500, 9501, 9502, 9503, 9504, 9505, 9506, 9507, 9508, 9509, 9510, 9511, 9512, 9513, 9514, 9515, 9516, 9517, 9518, 9519, 9520, 9521, 9522, 9523, 9524, 9525, 9526, 9527, 9528, 9529, 9530, 9531, 9532, 9533, 9534, 9535, 9536, 9537, 9538, 9539, 9540, 9541, 9542, 9543, 9544, 9545, 9546, 9547, 9548, 9549, 9550, 9551, 9552, 9553, 9554, 9555, 9556, 9557, 9558, 9559, 9560, 9561, 9562, 9563, 9564, 9565, 9566, 9567, 9568, 9569, 9570, 9571, 9572, 9573, 9574, 9575, 9576, 9577, 9578, 9579, 9580, 9581, 9582, 9583, 9584, 9585, 9586, 9587, 9588, 9589, 9590, 9591, 9592, 9593, 9594, 9595, 9596, 9597, 9598, 9599, 9600, 9601, 9602, 9603, 9604, 9605, 9606, 9607, 9608, 9609, 9610, 9611, 9612, 9613, 9614, 9615, 9616, 9617, 9618, 9619, 9620, 9621, 9622, 9623, 9624, 9625, 9626, 9627, 9628, 9629, 9630, 9631, 9632, 9633, 9634, 9635, 9636, 9637, 9638, 9639, 9640) c6(9641, 9642, 9643, 9644) c7(P, sY, bpH, T, dD, aSz, ea, uv, ht, DV, aw, cz, sM, n, bpk, fF, ah, aj, aqf, j, eq, aiC, ame, cDi, mC, J, Yh, ad, x, bpl, cfE, amc, M, jo, bU, hu, H, af, cDh, Vb, kl, MX, lR, A, sq, eZ, aq, YG, wd, jh, iR, f, uC, DL, el, qa, ek, ac, se, ag, vb, dW, dX, Km, fL, R, dy, bqo, ij, wx, fh, B, y, Ro); #c1(SERPIND1) c2(NP_000176) c3(heparin cofactor 2 precursor) c4(3842) c5(3843, 3844, 3845, 3846, 3847, 3848, 3849, 3850, 3851, 3852) c6( ) c7(T, dD, bpR, qb, acS, ag, ea, if, hl, aw, cDj, rf, aq, QL, eq, qN, is, uC, B, aQo); #c1(SERPINE1) c2(NP_000593) c3(plasminogen activator inhibitor 1 precursor) c4(13495, 13496, 13497) c5(13498, 13499, 13500, 13501, 13502, 13503, 13504, 13505, 13506, 13507, 13508, 13509, 13510, 13511, 13512, 13513, 13514, 13515) c6( ) c7(T, ea, bO, fd, ci, bRn, aY, GK, bv, aE, fm, hH, aa, bvD, az, ih, YG, aUG, axp, GR, wE, Zn, k, fy, aO, aj, cd, zo, TB, ey, bl, anP, sp, EU, fe, ajz, iO, dB, se, Fi, JQ, ajd, DM, jv, aA, cz, nS, AO, q, cy, Cy, dl, eu, cDo, wr, bf, bg, iy, lo, cl, cDk, pb, cT, rR, ac, CP, hr, tz, ao, aCA, dG, ape, ad, hX, tX, yt, vJ, bgr, lQ, wm, cH, cW, ff, dq, bpA, Sm, aFd, bz, ag, dX, lf, ne, oO, bM, lD, if, e, bB, sD, ip, x, vh, eG, fc, Du, hl, pD, as, cm, qJ, vA, mx, vy, wx, aFf, xN, dD, wu, n, aCZ, uE, no, ec, eV, hn, cY, hi, A, hQ, amo, sE, bpx, hL, at, ut, fL, B, wL, eX, fF, w, v, Do, V, eJ, hW, wN, rS, sl, Nw, nQ, ae, Lf, DN, bu, t, el, wO, gX, Gg, OR, eq, BY, py, od, qh, sj, d, NH, th, Z, bF, D, yE, cof, lI, gZ, MJ, gB, y, aC, fR, al, Xi, lR, M, K, dF, eL, Hl, aB, g, ve, bwe, uC, iP, bPC, uo, Hb, m, Uw, P, s, aw, qy, BH, ft, vC, gq, wb, Vb, MQ, sq, aUP, cDm, aZf, YF, cM, ar, kT, W, afc, mg, gv, gg, bNV, wj, fG, jx, Cg, j, aqf, eA, J, aHu, MR, hb, nX, bq, Fg, kl, Y, aBN, r, cfm, en, bN, QG, sn, c, hc, dh, dy, EY, fD, UF, Mj, gx, sY, am, akd, aZ, aET, ah, dP, fU, sN, al, ji, adx, uX, lU, lR, anX, xs, Fq, qa, ei, t, eb, crk, aR, il, qM, dQ, eN, hm, aW, hJ, aVc, eM, Jp, sU, uJ, mk, bqh, R, pw, xh, aWG, ez, fr, qN, ai, aDF, ql, ju, h, jy, dL, vx, wC, OV, aq, mo, op, cDn, Vy, vb, bm, qq, uz, bni, bL, eR, atB, dk, DV, sM, cR, C, cDl, ga, cdJ, qp, aT, af, s, oV, ajl, nM, pZ, wq, mf, hS, qX, qb, aD, vr, Fb, tK, mC, XB, KL, bPu, ib, sO, ts, Os, vw, jo, qt, aP, eZ, aF, uF, aDP, eK, LB, zr, bE, pE, z, uR, aS, be, pk, X, us); #c1(SERPINE2) c2(NP_001130000) c3(glia-derived nexin isoform b precursor) c4(52355) c5(52356, 52357, 52358, 52359, 52360, 52361, 52362, 52363, 52364, 52365, 52366, 52367, 52368, 52369, 52370, 52371, 52372, 52373, 52374, 52375, 52376, 52377, 52378, 52379, 52380, 52381, 52382, 52383, 52384, 52385, 52386) c6(52387, 52388) c7(bM, T, am, aC, Ke, cz, Fb, lR, pM, sD, q, av, aE, aet, aT, lG, af, wC, d, Ln, s, aW, ff, r, hp, en, as, cm, pb, ek, Zg, at, aS, mk, be, lC); #c1(SERPINE2) c2(NP_001130002) c3(glia-derived nexin isoform c precursor) c4(52389, 52390, 52391, 52392) c5(52393, 52394, 52395, 52396, 52397, 52398, 52399, 52400, 52401, 52402, 52403, 52404, 52405) c6( ) c7(bM, T, am, aC, Ke, cz, Fb, lR, pM, sD, q, av, aE, aet, aT, lG, af, wC, d, Ln, s, aW, ff, r, hp, en, as, cm, pb, ek, Zg, at, aS, mk, be, lC); #c1(SERPINE2) c2(NP_006207) c3(glia-derived nexin isoform a precursor) c4( ) c5(162849, 162850, 162851, 162852, 162853, 162854, 162855) c6( ) c7(bM, T, am, aC, Ke, cz, Fb, lR, pM, sD, q, av, aE, aet, aT, lG, af, wC, d, Ln, s, aW, ff, r, hp, en, as, cm, pb, ek, Zg, at, aS, mk, be, lC); #c1(SERPINE2) c2(XP_005246698) c3(glia-derived nexin isoform X2) c4(267371) c5(267372) c6( ) c7(bM, T, am, aC, Ke, cz, Fb, lR, pM, sD, q, av, aE, aet, aT, lG, af, wC, d, Ln, s, aW, ff, r, hp, en, as, cm, pb, ek, Zg, at, aS, mk, be, lC); #c1(SERPINE3) c2(NP_001094790) c3(serpin E3 precursor) c4(40712, 40713, 40714) c5(40715, 40716, 40717, 40718, 40719, 40720, 40721, 40722, 40723, 40724, 40725, 40726, 40727, 40728, 40729, 40730, 40731, 40732, 40733, 40734, 40735, 40736, 40737, 40738, 40739, 40740, 40741, 40742, 40743) c6(40744, 40745, 40746, 40747, 40748, 40749, 40750) c7(cd); #c1(SERPINF1) c2(NP_002606) c3(pigment epithelium-derived factor precursor) c4(130241) c5(130242, 130243, 130244, 130245, 130246, 130247) c6( ) c7(T, Tb, e, al, bB, aR, GK, aE, aeO, avA, K, EN, DA, bNp, aW, bvD, fc, hl, g, eF, iP, U, vy, nw, m, de, cZ, n, cQ, aj, qW, dd, acd, vx, Um, dx, cDp, aq, aUP, kp, mo, fs, iO, kT, W, bP, dB, bOj, Be, B, bL, v, eA, J, eJ, qK, aT, cy, zp, bq, ae, af, eu, jf, iF, zH, Y, r, en, t, c, JL, dy, auN, wi, uG, Ay, dG, ad, dP, hP, lQ, tt, d, lR, qt, ff, eH, iV, bz, gk, nb, bE, gZ, ei, oQ, GF, vt); #c1(SERPINF2) c2(NP_001159392) c3(alpha-2-antiplasmin isoform a precursor) c4(63926) c5(63927, 63928, 63929, 63930, 63931, 63932) c6( ) c7(T, cDr, aw, dQ, af, cDs, EU, ff, en, nN, cl, DT, Ew, cDq, fa, qq, dX, wx, m, ei, y, csf); #c1(SERPINF2) c2(NP_001159393) c3(alpha-2-antiplasmin isoform b precursor) c4(63933) c5(63934, 63935, 63936, 63937, 63938, 63939, 63940) c6( ) c7(T, cDr, aw, dQ, af, cDs, EU, ff, en, nN, cl, DT, Ew, cDq, fa, qq, dX, wx, m, ei, y, csf); #c1(SERPINF2) c2(XP_005256757) c3(alpha-2-antiplasmin isoform X5) c4(274328) c5(274329, 274330) c6( ) c7(T, cDr, aw, dQ, af, cDs, EU, ff, en, nN, cl, DT, Ew, cDq, fa, qq, dX, wx, m, ei, y, csf); #c1(SERPINF2) c2(XP_005256758) c3(alpha-2-antiplasmin isoform X4) c4( ) c5(274331, 274332, 274333, 274334, 274335, 274336, 274337, 274338, 274339, 274340, 274341, 274342, 274343, 274344, 274345, 274346, 274347, 274348, 274349, 274350, 274351, 274352, 274353, 274354, 274355, 274356, 274357, 274358, 274359, 274360, 274361, 274362, 274363, 274364, 274365, 274366, 274367, 274368, 274369, 274370, 274371, 274372, 274373, 274374, 274375, 274376, 274377, 274378, 274379, 274380, 274381, 274382, 274383, 274384, 274385, 274386, 274387, 274388, 274389, 274390, 274391, 274392, 274393, 274394, 274395, 274396, 274397, 274398, 274399, 274400, 274401, 274402, 274403, 274404, 274405, 274406, 274407, 274408, 274409, 274410, 274411, 274412, 274413, 274414, 274415, 274416, 274417, 274418, 274419, 274420, 274421, 274422, 274423, 274424, 274425, 274426, 274427, 274428, 274429, 274430, 274431, 274432, 274433, 274434, 274435, 274436, 274437, 274438, 274439, 274440, 274441, 274442, 274443, 274444, 274445, 274446, 274447, 274448, 274449, 274450, 274451, 274452, 274453, 274454, 274455, 274456, 274457, 274458, 274459, 274460, 274461, 274462, 274463, 274464, 274465, 274466, 274467, 274468, 274469, 274470, 274471, 274472, 274473, 274474, 274475, 274476, 274477, 274478) c6(274479) c7(T, cDr, aw, dQ, af, cDs, EU, ff, en, nN, cl, DT, Ew, cDq, fa, qq, dX, wx, m, ei, y, csf); #c1(SERPING1) c2(NP_000053) c3(plasma protease CI inhibitor precursor) c4(836, 837, 838, 839, 840) c5(841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021) c6(1022, 1023, 1024, 1025, 1026, 1027, 1028, 1029, 1030, 1031) c7(bM, cDu, DM, iW, aw, bWQ, cDv, aoE, OY, fu, C, zo, baA, i, ds, H, acd, Um, af, tN, Oj, EU, bWS, eZ, aq, lo, pD, t, cM, cDt, cm, dr, kT, W, nb, axs, Qu, RJ, Km, GF, B, hv, L); #c1(SERPINH1) c2(XP_011543629) c3 (serpin H1 isoform X1) c4(342637, 342638, 342639, 342640, 342641, 342642, 342643, 342644, 342645, 342646) c5(342647, 342648, 342649, 342650, 342651, 342652, 342653, 342654, 342655, 342656, 342657, 342658, 342659, 342660, 342661, 342662, 342663, 342664, 342665, 342666, 342667, 342668, 342669, 342670, 342671, 342672, 342673, 342674, 342675, 342676, 342677, 342678, 342679, 342680, 342681, 342682, 342683, 342684, 342685, 342686, 342687, 342688, 342689, 342690, 342691, 342692, 342693, 342694, 342695, 342696, 342697, 342698, 342699, 342700, 342701, 342702, 342703, 342704, 342705, 342706, 342707, 342708, 342709, 342710, 342711, 342712, 342713, 342714, 342715, 342716, 342717, 342718, 342719, 342720, 342721, 342722, 342723, 342724, 342725, 342726, 342727, 342728, 342729, 342730, 342731, 342732, 342733, 342734, 342735, 342736, 342737, 342738) c6(342739, 342740, 342741, 342742, 342743, 342744, 342745, 342746, 342747, 342748, 342749, 342750, 342751, 342752, 342753, 342754, 342755, 342756, 342757, 342758, 342759, 342760, 342761, 342762) c7(T, wi, cDw, fR, sQ, e, al, aA, aTy, j, ci, eA, tX, ds, kH, Ne, cy, rB, K, lQ, zp, H, azh, je, aRL, aYp, fQ, aF, aDP, iV, eE, cm, op, ck, LB, iM, bJa, vA, ajd, uo, aDJ, ut, y, cn, L); #c1(SERPINI1) c2(NP_005016) c3(neuroserpin precursor) c4(152186, 152187, 152188) c5(152189, 152190, 152191, 152192, 152193, 152194, 152195, 152196, 152197, 152198, 152199, 152200, 152201, 152202, 152203, 152204, 152205, 152206, 152207, 152208, 152209, 152210) c6( ) c7(axS, T, ke, d, ac, hb, dB, M, U, aA, yZ, aF, cbR, yU, V, ae, af, ad, ek, cB); #c1(SERPINI2) c2(NP_001012303) c3(serpin 12 isoform 1 precursor) c4(24875, 24876) c5(24877, 24878, 24879, 24880, 24881) c6( ) c7(cDx, Bd, T, Be); #c1(SERPINI2) c2(NP_006208) c3(serpin 12 isoform 2 precursor) c4( ) c5(162856, 162857, 162858, 162859, 162860, 162861, 162862, 162863, 162864, 162865, 162866, 162867, 162868, 162869, 162870, 162871, 162872, 162873, 162874, 162875, 162876, 162877, 162878, 162879, 162880, 162881, 162882, 162883, 162884) c6(162885, 162886, 162887, 162888, 162889, 162890, 162891, 162892, 162893, 162894) c7(cDx, Bd, T, Be); #c1(SERTAD1) c2(NP_037508) c3(SERTA domain-containing protein 1) c4(175919) c5(175920, 175921, 175922, 175923, 175924, 175925) c6( ) c7(T, cA, bE, aW, ff, aT, bB, m, en, t, it, X); #c1(SERTAD2) c2(XP_011531506) c3(SERTA domain-containing protein 2 isoform X2) c4( ) c5(327654, 327655) c6( ) c7(T, B, q); #c1(SERTAD2) c2(XP_011531507) c3(SERTA domain-containing protein 2 isoform X1) c4(327656) c5(327657, 327658, 327659, 327660, 327661) c6( ) c7(T, B, q); #c1(SESN1) c2(NP_001186862) c3 (sestrin-1 isoform 2) c4(78715) c5(78716, 78717, 78718, 78719, 78720, 78721, 78722) c6( ) c7(bL, BV, K, wy); #c1(SESN1) c2(NP_001186863) c3(sestrin-1 isoform 3) c4( ) c5(78723, 78724, 78725, 78726, 78727) c6( ) c7(bL, BV, K, wy); #c1(SESN1) c2(NP_055269) c3(sestrin-1 isoform 1) c4(180182, 180183) c5(180184, 180185, 180186, 180187, 180188, 180189) c6( ) c7(bL, BV, K, wy); #c1(SESN3) c2(NP_001258523) c3(sestrin-3 isoform 2) c4(96103) c5(96104, 96105, 96106, 96107, 96108, 96109) c6( ) c7(bL, T, VZ, fc, akY, cP, V); #c1(SESN3) c2(NP_653266) c3(sestrin-3 isoform 1) c4(239391, 239392, 239393) c5(239394, 239395, 239396, 239397, 239398, 239399, 239400, 239401, 239402, 239403, 239404, 239405, 239406, 239407, 239408, 239409, 239410, 239411) c6( ) c7(bL, T, VZ, fc, akY, cP, V); #c1(SET) c2(NP_001234929) c3(protein SET isoform 3) c4(87811) c5(87812, 87813, 87814, 87815, 87816, 87817) c6( ) c7(T, id, btY, oA, fd, jh, fr, q, ad, er, axC, av, aE, oW, aT, h, bK, jj, rz, ae, af, gj, kl, iU, aW, Y, r, gm, ik, ck, cDy, W, bt, kS); #c1(SET) c2(NP_001234930) c3(protein SET isoform 4) c4(87818, 87819) c5(87820, 87821, 87822, 87823, 87824, 87825, 87826, 87827, 87828, 87829, 87830, 87831) c6( ) c7(T, id, btY, oA, fd, jh, fr, q, ad, er, axC, av, aE, oW, aT, h, bK, jj, rz, ae, af, gj, kl, il, aW, Y, r, gm, ik, ck, cDy, W, bt, kS); #c1(SET) c2(NP_003002) c3(protein SET isoform 2) c4(134339) c5(134340, 134341, 134342, 134343, 134344, 134345) c6( ) c7(T, id, btY, oA, fd, jh, fr, q, ad, er, axC, av, aE, oW, aT, h, bK, jj, rz, ae, af, gj, kl, il, aW, Y, r, gm, iK, ck, cDy, W, bt, kS); #c1(SET) c2(XP_011517213) c3(protein SET isoform X1) c4(310801) c5(310802, 310803, 310804, 310805, 310806, 310807, 310808, 310809, 310810) c6( ) c7(T, id, btY, oA, fd, jh, fr, q, ad, er, axC, av, aE, oW, aT, h, bK, jj, rz, ae, af, gj, kl, iU, aW, Y, r, gm, ik, ck, cDy, W, bt, kS); #c1(SETBP1) c2(NP_001123582) c3(SET-binding protein isoform b) c4(47775) c5(47776, 47777, 47778, 47779, 47780, 47781, 47782, 47783, 47784, 47785, 47786, 47787) c6( ) c7(bL, T, LX, atS, aj, nA, hF, br, ad, x, brH, Ko, aQM, pq, iU, Az, EZ, BE, bw, fq, bN, ck, W, iT, eB, pg, bim, hE, bt, LT); #c1(SETBP1) c2(NP_056374) c3(SET-binding protein isoform a) c4(187334, 187335) c5(187336, 187337, 187338, 187339, 187340, 187341) c6( ) c7(bL, T, LX, atS, aj, nA, hF, br, ad, x, brH, Ko, aQM, pq, il, Az, EZ, eE, bw, fq, bN, ck, W, iT, eB, pg, bim, hE, bt, LT); #c1(SETD1A) c2(NP_055527) c3(histone-lysine N-methyltransferase SETD1A) c4(181766, 181767, 181768) c5(181769, 181770, 181771, 181772, 181773, 181774, 181775, 181776, 181777, 181778, 181779, 181780, 181781, 181782, 181783, 181784, 181785, 181786, 181787) c6( ) c7(T, bB, hy, BF, hF, Gt, ld, h, ne, BN, BO, ff, dg, en, g, t, bz, ck, sS, eM, bE, CZ, m, y, iu); #c1(SETD1B) c2(NP_055863) c3(histone-lysine N-methyltransferase SETD1B) c4(183986) c5(183987, 183988, 183989, 183990, 183991, 183992, 183993, 183994, 183995, 183996, 183997) c6( ) c7(ff, pm, en, r); #c1(SETD2) c2(NP_054878) c3(histone-lysine N-methyltransferase SETD2) c4(178162, 178163, 178164) c5(178165, 178166, 178167, 178168, 178169, 178170, 178171, 178172, 178173, 178174, 178175, 178176, 178177, 178178, 178179, 178180, 178181, 178182) c6( ) c7(oO, T, ea, if, fd, bB, ci, aE, K, hu, aCU, cp, aW, fc, dg, g, as, cm, iP, y, eM, iA, tG, nw, m, dD, aw, NT, qy, n, fr, qN, rP, cd, md, rC, h, Nf, cY, hQ, fe, aq, kp, YF, bo, qF, B, fG, aA, j, lu, q, eA, eJ, oT, yw, ld, qK, aT, cy, ae, s, nO, Y, r, en, t, ik, pb, ck, nM, kP, ac, dy, VB, Kd, jh, vr, dG, eq, hF, ad, Jm, hd, py, Db, oW, ji, d, BO, et, ff, aF, iV, iR, bz, ek, es, pc, bE, aS, lC, tP, iu, bEt); #c1(SETD3) c2(XP_011535533) c3(histone-lysine N-methyltransferase setd3 isoform X3) c4(332595) c5(332596) c6( ) c7(jh); #c1(SETD3) c2(XP_011535534) c3(histone-lysine N-methyltransferase setd3 isoform X1) c4( ) c5(332597, 332598, 332599, 332600, 332601) c6( ) c7(jh); #c1(SETD3) c2(XP_011535537) c3(histone-lysine N-methyltransferase setd3 isoform X2) c4(332602) c5(332603, 332604, 332605, 332606, 332607, 332608) c6( ) c7(jh); #c1(SETD5) c2(NP_001073986) c3(SET domain-containing protein 5 isoform 1) c4(36794) c5(36795, 36796, 36797, 36798, 36799, 36800, 36801, 36802, 36803, 36804, 36805, 36806, 36807, 36808, 36809, 36810) c6( ) c7(cDz, nA); #c1(SETD5) c2(NP_001278972) c3(SET domain-containing protein 5 isoform 2) c4(111156) c5(111157, 111158, 111159, 111160, 111161, 111162, 111163, 111164) c6( ) c7(cDz, nA); #c1(SETD5) c2(XP_011532230) c3(SET domain-containing protein 5 isoform X6) c4(328292) c5(328293, 328294) c6( ) c7(cDz, nA); #c1(SETD7) c2(NP_085151) c3(histone-lysine N-methyltransferase SETD7) c4(219481) c5(219482, 219483, 219484, 219485, 219486, 219487) c6( ) c7(T, ls, B, A, aj, dG, ae, q, J, ad); #c1(SETD8) c2(NP_065115) c3(N-lysine methyltransferase SETD8) c4(203992) c5(203993, 203994, 203995, 203996, 203997, 203998, 203999, 204000, 204001, 204002) c6( ) c7(T, eM, aW, aT, K, B); #c1(SETDB2) c2(NP_001153780) c3(histone-lysine N-methyltransferase SETDB2 isoform b) c4(60998) c5(60999, 61000, 61001, 61002, 61003, 61004) c6( ) c7(buV, mQ, bM, r, cj); #c1(SETDB2) c2(NP_114121) c3(histone-lysine N-methyltransferase SETDB2 isoform a) c4(222234) c5(222235, 222236, 222237, 222238, 222239, 222240, 222241) c6( ) c7(buV, ml, bM, r, cj); #c1(SETMAR) c2(NP_001263254) c3(histone-lysine N-methyltransferase SETMAR isoform 3) c4(97089) c5(97090, 97091, 97092, 97093, 97094, 97095, 97096, 97097, 97098, 97099, 97100) c6( ) c7(bt); #c1(SETMAR) c2(NP_006506) c3(histone-lysine N-methyltransferase SETMAR isoform 1) c4(165875, 165876) c5(165877, 165878, 165879, 165880, 165881, 165882) c6( ) c7(bt); #c1(SETMAR) c2(XP_006713355) c3(histone-lysine N-methyltransferase SETMAR isoform X1) c4( ) c5(287161) c6( ) c7(bt); #c1(SETMAR) c2(XP_006713357) c3(histone-lysine N-methyltransferase SETMAR isoform X2) c4( ) c5(287162) c6( ) c7(bt); #c1(SETX) c2(NP_055861) c3(probable helicase senataxin) c4(183972, 183973) c5(183974, 183975, 183976, 183977, 183978, 183979, 183980, 183981, 183982, 183983, 183984, 183985) c6( ) c7(ads, OI, qy, TM, fu, bX, cDA, ado, aR, bMh, kf, aU, bl, DG, K, KU, adr, KH, eZ, EH, dr, adq, ky, kB, c, U, KA, bC, KF); #c1(SETX) c2(XP_011516707) c3(probable helicase senataxin isoform X1) c4(310158, 310159) c5(310160, 310161, 310162, 310163, 310164, 310165, 310166, 310167, 310168, 310169, 310170, 310171) c6( ) c7(ads, Ol, qy, TM, fu, bX, cDA, ado, aR, bMh, kf, aU, bl, DG, K, KU, adr, KH, eZ, EH, dr, adq, ky, kB, c, U, KA, bC, KF); #c1(SETX) c2(XP_011516710) c3(probable helicase senataxin isoform X4) c4( ) c5(310172) c6( ) c7(ads, Ol, qy, TM, fu, bX, cDA, ado, aR, bMh, kf, aU, bl, DG, K, KU, adr, KH, eZ, EH, dr, adq, ky, kB, c, U, KA, bC, KF); #c1(SEZ6) c2(NP_001092105) c3(seizure protein 6 homolog isoform 2 precursor) c4(39388, 39389) c5(39390, 39391, 39392, 39393, 39394, 39395) c6( ) c7(n, ha); #c1(SEZ6) c2(NP_001277131) c3(seizure protein 6 homolog isoform 3) c4( ) c5(109107, 109108, 109109, 109110, 109111, 109112) c6( ) c7(n, ha); #c1(SEZ6) c2(NP_849191) c3(seizure protein 6 homolog isoform 1 precursor) c4(254473, 254474) c5(254475, 254476, 254477, 254478, 254479, 254480, 254481) c6( ) c7(n, ha); #c1(SEZ6) c2(XP_011522619) c3(seizure protein 6 homolog isoform X3) c4( ) c5(317174, 317175, 317176, 317177, 317178, 317179) c6( ) c7(n, ha); #c1(SEZ6L) c2(NP_001171702) c3(seizure 6-like protein isoform 2 precursor) c4(71332) c5(71333, 71334, 71335, 71336, 71337, 71338) c6( ) c7(av, ft, A, eM, bK, j, bz); #c1(SEZ6L) c2(NP_001171703) c3(seizure 6-like protein isoform 3 precursor) c4(71339) c5(71340, 71341, 71342, 71343, 71344, 71345) c6( ) c7(av, ft, A, eM, bK, j, bz); #c1(SEZ6L) c2(NP_001171704) c3(seizure 6-like protein isoform 4 precursor) c4(71346) c5( ) c6( ) c7(av, ft, A, eM, bK, j, bz); #c1(SEZ6L) c2(NP_001171705) c3(seizure 6-like protein isoform 5 precursor) c4(71347) c5(71348, 71349, 71350, 71351, 71352, 71353) c6( ) c7(av, ft, A, eM, bK, j, bz); #c1(SEZ6L) c2(NP_001171706) c3(seizure 6-like protein isoform 6 precursor) c4(71354) c5(71355, 71356, 71357, 71358, 71359, 71360) c6( ) c7(av, ft, A, eM, bK, j, bz); #c1(SEZ6L) c2(NP_066938) c3(seizure 6-like protein isoform 1 precursor) c4(207594) c5(207595, 207596, 207597, 207598, 207599, 207600) c6( ) c7(av, ft, A, eM, bK, j, bz); #c1(SEZ6L) c2(XP_005261496) c3(seizure 6-like protein isoform X2) c4( ) c5(277397, 277398, 277399, 277400, 277401, 277402) c6( ) c7(av, ft, A, eM, bK, j, bz); #c1(SEZ6L2) c2(NP_001107572) c3(seizure 6-like protein 2 isoform 4 precursor) c4(42875) c5(42876, 42877, 42878, 42879, 42880, 42881) c6( ) c7(ft, cbH, AQ, UF, aA, aF, K, Uw, rz, bz, af, bN, L); #c1(SEZ6L2) c2(NP_001230262) c3(seizure 6-like protein 2 isoform 6 precursor) c4(86170) c5(86171, 86172, 86173, 86174, 86175, 86176) c6( ) c7(ft, cbH, AQ, UF, aA, aF, K, Uw, rz, bz, af, bN, L); #c1(SEZ6L2) c2(NP_036542) c3(seizure 6-like protein 2 isoform 1 precursor) c4(174307) c5(174308, 174309, 174310, 174311, 174312, (74313) c6( ) c7(ft, cbH, AQ, UF, aA, aF, K, Uw, rz, bz, af, bN, L); #c1(SEZ6L2) c2(NP_963869) c3(seizure 6-like protein 2 isoform 2 precursor) c4(262481) c5(262482, 262483, 262484, 262485, 262486, 262487) c6( ) c7(ft, cbH, AQ, UF, aA, aF, K, Uw, rz, bz, af, bN, L); #c1(SF1) c2(NP_001171501) c3(splicing factor 1 isoform 6) c4(70326) c5( ) c6( ) c7(nD, T, pY, Y, r, iv, cy, t, q); #c1(SF1) c2(NP_004621) c3(splicing factor 1 isoform 1) c4(148270, 148271) c5( ) c6( ) c7(nD, T, pY, Y, r, iv, cy, t, q); #c1(SF1) c2(NP_973724) c3(splicing factor 1 isoform 2) c4(262636) c5(262637, 262638, 262639, 262640, 262641, 262642) c6( ) c7(nD, T, pY, Y, r, iv, cy, t, q); #c1(SF1) c2(NP_973726) c3(splicing factor 1 isoform 4) c4(262643) c5(262644, 262645, 262646, 262647, 262648, 262649) c6( ) c7(nD, T, pY, Y, r, iv, cy, t, q); #c1(SF1) c2(NP_973727) c3(splicing factor 1 isoform 3) c4(262650, 262651) c5(262652, 262653, 262654, 262655, 262656, 262657, 262658, 262659, 262660, 262661, 262662, 262663, 262664, 262665, 262666, 262667, 262668, 262669, 262670, 262671, 262672, 262673, 262674, 262675, 262676, 262677, 262678, 262679, 262680, 262681, 262682) c6( ) c7(nD, T, pY, Y, r, iv, cy, t, q); #c1(SF3A1) c2(NP_005868) c3(splicing factor 3A subunit 1) c4(160018, 160019, 160020) c5(160021, 160022, 160023, 160024, 160025, 160026, 160027, 160028, 160029, 160030, 160031, 160032, 160033, 160034, 160035, 160036, 160037, 160038) c6( ) c7(aT); #c1(SF3B1) c2(NP_001005526) c3 (splicing factor 38 subunit 1 isoform 2) c4(21816) c5(21817, 21818, 21819, 21820, 21821, 21822, 21823, 21824, 21825, 21826) c6( ) c7(T, afA, Js, qx, aDT, qy, fZ, bs, n, ol, aeu, oY, fd, jh, abd, hF, q, br, ad, x, OB, oW, aT, aU, vw, dz, h, jj, pq, Nr, oP, iU, aix, hG, aW, Y, et, gK, pr, r, Mz, kD, po, pm, hf, bw, aiv, iP, Nq, yd, ck, iO, pc, UY, iT, kz, cj, pg, dW, k, bt, Jo); #c1(SF3B1) c2(NP_036565) c3(splicing factor 3B subunit 1 isoform 1) c4(174434) c5(174435, 174436, 174437, 174438, 174439, 174440) c6( ) c7(T, afA, Js, qx, aDT, qy, fZ, bs, n, ol, aeu, oY, fd, jh, abd, hF, q, br, ad, x, oB, oW, aT, aU, vw, dz, h, jj, pq, Nr, oP, iU, aix, hG, aW, Y, et, gK, pr, r, Mz, kD, po, pm, hf, bw, aiv, iP, Nq, yd, ck, iO, pc, UY, iT, kz, cj, pg, dW, k, bt, Jo); #c1(SF3B2) c2(NP_006833) c3(splicing factor 3B subunit 2) c4(168518) c5(168519, 168520, 168521, 168522, 168523, 168524, 168525, 168526, 168527, 168528, 168529, 168530) c6( ) c7(T, oP, afA, kz, Y, aDT, jh, bs, cj, kD, bw, hf, iP, bt, br, ad); #c1(SF3B2) c2(XP_005273783) c3(splicing factor 3B subunit 2 isoform X1) c4(284834) c5( ) c6( ) c7(T, oP, afA, kz, Y, aDT, jh, bs, cj, kD, bw, hf, iP, bt, br, ad); #c1(SF3B6) c2(NP_057131) c3(splicing factor 38 subunit 6) c4(189067) c5(189068, 189069, 189070, 189071, 189072, 189073) c6( ) c7(sc, T, aEj, jh, fr, ci, q, ad, ft, aT, dL, K, jf, aW, Y, r, fe, aF, g, bF, cj, EY, B); #c1(SFI1) c2(NP_001007468) c3(protein SFI1 homolog isoform a) c4(22714) c5(22715, 22716, 22717, 22718, 22719, 22720, 22721, 22722, 22723, 22724, 22725, 22726) c6( ) c7(aY, eR); #c1(SFI1) c2(NP_001245254) c3(protein SFI1 homolog isoform c) c4(92883) c5(92884, 92885, 92886, 92887, 92888, 92889) c6( ) c7(aY, eR); #c1(SFI1) c2(NP_001245255) c3(protein SFI1 homolog isoform d) c4(92890, 92891) c5(92892, 92893, 92894, 92895, 92896, 92897, 92898, 92899, 92900, 92901) c6( ) c7(aY, eR); #c1(SFI1) c2(NP_055590) c3(protein SFI1 homolog isoform b) c4(182145) c5(182146, 182147, 182148, 182149, 182150, 182151) c6( ) c7(aY, eR); #c1(SFI1) c2(XP_011528887) c3(protein SFI1 homolog isoform X12) c4(324271) c5(324272, 324273, 324274, 324275, 324276, 324277) c6( ) c7(aY, eR); #c1(SFI1) c2(XP_011528888) c3(protein SFI1 homolog isoform X13) c4(324278) c5(324279, 324280, 324281, 324282, 324283, 324284, 324285) c6( ) c7(aY, eR); #c1(SFMBT1) c2(NP_057413) c3(scm-like with four MBT domains protein 1) c4(190634, 190635, 190636, 190637, 190638) c5(190639, 190640, 190641, 190642, 190643, 190644, 190645, 190646, 190647, 190648, 190649, 190650, 190651, 190652, 190653, 190654, 190655, 190656, 190657, 190658, 190659, 190660, 190661, 190662, 190663, 190664, 190665, 190666, 190667, 190668, 190669, 190670, 190671, 190672, 190673, 190674, 190675, 190676) c6( ) c7(dB, T, fq, fr, cz); #c1(SFMBT2) c2(NP_001018049) c3(scm-like with four MBT domains protein 2) c4(26915, 26916, 26917) c5(26918, 26919, 26920, 26921, 26922, 26923, 26924, 26925, 26926, 26927, 26928, 26929, 26930, 26931, 26932, 26933, 26934, 26935, 26936, 26937, 26938, 26939, 26940, 26941, 26942, 26943) c6( ) c7(ac, eZ); #c1(SFR1) c2(NP_001002759) c3(swi5-dependent recombination DNA repair protein 1 homolog isoform a) c4(19592, 19593) c5(19594, 19595, 19596, 19597, 19598, 19599, 19600) c6( ) c7(aW, T, aT); #c1(SFR1) c2(NP_660290) c3(swi5-dependent recombination DNA repair protein 1 homolog isoform b) c4( ) c5(240664, 240665, 240666, 240667, 240668) c6( ) c7(aW, T, aT); #c1(SFR1) c2(XP_005269578) c3(swi5-dependent recombination DNA repair protein 1 homolog isoform X1) c4(282648) c5(282649, 282650, 282651, 282652, 282653, 282654) c6( ) c7(aW, T, aT); #c1(SFRP1) c2(NP_003003) c3(secreted frizzled-related protein 1 precursor) c4(134346) c5(134347, 134348, 134349, 134350, 134351, 134352, 134353, 134354, 134355, 134356) c6( ) c7(bM, T, os, aC, pf, if, fd, bB, ci, ip, aE, qM, eL, aW, LZ, g, as, cm, eM, iA, cDB, go, vA, cj, Hb, m, in, n, fr, qN, Bl, ft, jz, dL, sp, fe, aq, mo, dw, op, Be, it, B, jx, aA, bJw, v, C, AO, q, er, eA, MR, hW, aT, jj, ae, bA, jf, s, iU, r, en, cl, apx, t, pb, kP, dy, Bd, bt, vi, id, oZ, ad, hX, dP, av, py, gj, d, ff, aF, iV, bz, bF, es, amS, zr, bE, ei, y, kS, iu); #c1(SFRP2) c2(NP_003004) c3(secreted frizzled-related protein 2 precursor) c4(134357) c5(134358, 134359, 134360, 134361, 134362, 134363) c6( ) c7(bM, T, aC, if, aw, EQ, aA, fd, axk, fr, ci, qN, q, ad, eA, zw, Bc, aT, dL, ae, YT, pl, sj, jf, d, s, aW, ff, r, fe, aq, aF, en, g, apx, t, nC, fq, iV, es, YE, iA, bkQ, gX, cj, hR, zl, bt); #c1(SFRP4) c2(NP_003005) c3(secreted frizzled-related protein 4 precursor) c4(134364, 134365) c5(134366, 134367, 134368, 134369, 134370, 134371, 134372, 134373, 134374, 134375, 134376, 134377, 134378, 134379, 134380, 134381) c6(134382, 134383, 134384, 134385) c7(bL, gh, T, aC, if, NT, j, ci, qN, fr, q, ad, dP, amB, dL, ae, bA, s, aW, ff, r, aq, en, t, pb, zV, es, YE, hR, adR, m, azr, Th, bt, B); #c1(SFRP5) c2(NP_003006) c3(secreted frizzled-related protein 5 precursor) c4(134386) c5(134387, 134388, 134389, 134390, 134391, 134392) c6( ) c7(bL, bM, T, aC, aD, if, aA, fd, n, aj, v, fr, qN, hF, q, J, ad, hX, Xc, aT, dL, ae, dx, nO, aW, ff, fc, r, aq, aF, en, cl, g, t, apx, ck, es, bF, sS, amS, pc, eM, iA, mf, m, cP, y, bt, B); #c1(SFSWAP) c2(NP_001248340) c3(splicing factor, suppressor of white-apricot homolog isoform 1) c4(93715, 93716) c5(93717, 93718, 93719, 93720, 93721, 93722) c6( ) c7(cd, VX); #c1(SFSWAP) c2(NP_004583) c3(splicing factor, suppressor of white-apricot homolog isoform 2) c4(147887) c5(147888, 147889, 147890, 147891, 147892, 147893) c6( ) c7(cd, VX); #c1(SFSWAP) c2(XP_011536957) c3(splicing factor, suppressor of white-apricot homolog isoform X3) c4( ) c5(334644, 334645, 334646, 334647, 334648, 334649) c6( ) c7(cd, VX); #c1(SFT2D2) c2(NP_955376) c3(vesicle transport protein SFT2B) c4(261597, 261598) c5(261599, 261600, 261601, 261602, 261603, 261604, 261605, 261606, 261607, 261608, 261609, 261610, 261611, 261612, 261613, 261614, 261615) c6( ) c7(bA, cz); #c1(SFT2D3) c2(NP_116129) c3(vesicle transport protein SFT2C) c4(225458) c5(225459, 225460, 225461, 225462, 225463, 225464) c6( ) c7(q, r); #c1(SFTA2) c2(NP_995326) c3(surfactant-associated protein 2 precursor) c4(263855, 263856) c5(263857, 263858, 263859, 263860, 263861, 263862, 263863, 263864, 263865, 263866, 263867, 263868, 263869, 263870, 263871, 263872, 263873, 263874, 263875, 263876, 263877, 263878, 263879, 263880, 263881, 263882, 263883, 263884, 263885, 263886, 263887) c6(263888, 263889, 263890, 263891, 263892) c7(hH, ft, eZ); #c1(SFTA3) c2(NP_001094811) c3(surfactant-associated protein 3) c4(40765) c5(40766, 40767, 40768, 40769, 40770, 40771) c6( ) c7(cm); #c1(SFTPA1) c2(NP_001158117) c3(pulmonary surfactant-associated protein A1 isoform 3 precursor) c4(63155) c5(63156, 63157, 63158, 63159, 63160, 63161) c6( ) c7(bM, T, Dw, aGC, fF, aBo, fA, br, wa, AK, av, ft, aaU, aMn, bW, oW, cU, jy, SD, aa, afu, eG, iU, ff, ap, pD, EZ, en, g, ajz, bw, bz, LB, vl, eM, Nk, bOj, PV, dT, uJ, at, sZ, iu, ao); #c1(SFTPA1) c2(XP_005270119) c3(pulmonary surfactant-associated protein A1 isoform X2) c4(283111, 283112, 283113) c5(283114, 283115, 283116, 283117, 283118, 283119, 283120, 283121, 283122, 283123, 283124, 283125, 283126, 283127, 283128, 283129, 283130, 283131, 283132, 283133, 283134, 283135, 283136, 283137, 283138, 283139, 283140, 283141, 283142, 283143) c6( ) c7(bM, T, Dw, aGC, fF, aBo, fA, br, wa, AK, av, ft, aaU, aMn, bW, oW, cU, jy, SD, aa, afu, eG, iU, ff, ap, pD, EZ, en, g, ajz, bw, bz, LB, vU, eM, Nk, bOj, PV, dT, uJ, at, sZ, iu, ao); #c1(SFTPA1) c2(XP_006718016) c3(pulmonary surfactant-associated protein A1 isoform X1) c4(291448, 291449) c5(291450, 291451, 291452, 291453, 291454, 291455, 291456, 291457, 291458, 291459) c6( ) c7(bM, T, Dw, aGC, fF, aBo, fA, br, wa, AK, av, ft, aaU, aMn, bW, oW, cU, jy, SD, aa, afu, eG, iU, ff, ap, pD, EZ, en, g, ajz, bw, bz, LB, vU, eM, Nk, bOj, PV, dT, uJ, at, sZ, iu, ao); #c1(SFTPA2) c2(XP_005270185) c3 (pulmonary surfactant-associated protein A2 isoform X2) c4(283202, 283203) c5(283204, 283205, 283206, 283207, 283208) c6( ) c7(bM, T, Dw, fG, fF, aBo, v, fA, atK, ad, wD, AK, av, ft, aaU, aMn, bW, Lv, cU, jy, K, SD, ae, aa, afu, eG, mR, ap, pD, EZ, g, ajz, bz, LB, vU, eM, Nk, boj, PV, at, uJ, fD, y, sZ, iu); #c1(SFTPA2) c2(XP_005270189) c3(pulmonary surfactant-associated protein A2 isoform X1) c4( ) c5(283209, 283210, 283211, 283212, 283213, 283214, 283215, 283216, 283217, 283218, 283219, 283220, 283221) c6( ) c7(bM, T, Dw, fG, fF, aBo, v, fA, atK, ad, wD, AK, av, ft, aaU, aMn, bW, Lv, cU, jy, K, SD, ae, aa, afu, eG, mR, ap, pD, EZ, g, ajz, bz, LB, vU, eM, Nk, boj, PV, at, uJ, fD, y, sZ, iu); #c1(SFTPB) c2(XP_005264544) c3(pulmonary surfactant-associated protein B isoform X1) c4(279016, 279017, 279018, 279019) c5(279020, 279021, 279022, 279023, 279024, 279025, 279026, 279027, 279028, 279029, 279030, 279031, 279032, 279033, 279034, 279035, 279036, 279037, 279038, 279039, 279040, 279041, 279042, 279043, 279044, 279045, 279046) c6( ) c7(bM, T, cDC, Dw, am, fR, fG, fF, blB, v, C, fA, atK, fw, sD, ad, av, fE, co, bW, Lv, jy, Ds, SD, ae, Gc, aa, fx, afe, ap, g, bz, pb, dr, LB, qJ, eM, aWV, TJ, Qu, wk, uR, at, dT, uJ, fC, fD, lC, iu, ao); #c1(SFTPC) c2(NP_001165828) c3 (pulmonary surfactant-associated protein C isoform 2 proprotein) c4(67906) c5(67907, 67908, 67909, 67910, 67911, 67912) c6( ) c7(lD, bM, T, kh, Dw, fR, Zl, fG, fF, ci, fA, lK, fw, atK, av, fE, brn, ft, bW, Lv, lG, Ds, cU, jy, K, MO, aa, ly, kl, fx, ap, lo, aDP, g, crG, lx, LB, axS, eM, aWV, wk, uR, ajd, adl, fz, dT, uJ, fC, at, fl, cDD, fD, buO, lC, iu); #c1(SFTPC) c2(NP_001165881) c3(pulmonary surfactant-associated protein C isoform 1 proprotein) c4(67913) c5(67914, 67915, 67916, 67917, 67918, 67919) c6( ) c7(lD, bM, T, kh, Dw, fR, Zl, fG, fF, ci, fA, lK, fw, atK, av, fE, brn, ft, bW, Lv, lG, Ds, cU, jy, K, MO, aa, ly, kl, fx, ap, lo, aDP, g, crG, lx, LB, axS, eM, aWV, wk, uR, ajd, adl, fz, dT, uJ, fC, at, fl, cDD, fD, buO, lC, iu); #c1(SFTPD) c2(NP_003010) c3(pulmonary surfactant-associated protein D precursor) c4(134407, 134408) c5(134409, 134410, 134411, 134412, 134413, 134414, 134415, 134416, 134417, 134418, 134419, 134420) c6( ) c7(bL, bM, T, dD, Dw, fR, Zl, ea, fG, oe, rl, fF, j, eq, C, pM, atK, ai, wa, av, aaU, ds, bW, Lv, cU, Ds, dL, h, K, aDp, eN, qE, aa, cCX, fx, lx, sp, ff, mQ, sP, eZ, rM, fe, pD, uF, en, g, iV, EZ, bz, LB, TA, cT, iQ, Ui, eM, z, Ny, bOj, at, uz, wR, dT, fD, du, y, ao); #c1(SFXN1) c2(NP_073591) c3(sideroflexin-1) c4(213001, 213002, 213003) c5(213004, 213005, 213006, 213007, 213008, 213009, 213010, 213011, 213012, 213013, 213014, 213015, 213016, 213017, 213018, 213019, 213020, 213021) c6( ) c7(bL, oq, am, id, al, n, ci, nA, ad, hX, qC, Bl, hd, aT, dL, ae, eL, N, aW, ff, hl, aJL, cr, kt, en, mv, g, bcR, qe, ER); #c1(SFXN2) c2(NP_849189) c3(sideroflexin-2) c4( ) c5(254461, 254462, 254463, 254464, 254465) c6( ) c7(an); #c1(SFXN2) c2(XP_011537563) c3(sideroflexin-2 isoform X2) c4(335152) c5(335153) c6( ) c7(an); #c1(SFXN4) c2(NP_998814) c3(sideroflexin-4) c4(266025, 266026, 266027) c5(266028, 266029, 266030, 266031, 266032, 266033, 266034) c6( ) c7(cDE, kC, Gq, UP); #c1(SFXN4) c2(XP_005269582) c3(sideroflexin-4 isoform X1) c4(282655, 282656) c5(282657, 282658, 282659, 282660, 282661, 282662, 282663) c6( ) c7(cDE, kC, Gq, UP); #c1(SFXN4) c2(XP_005269584) c3(sideroflexin-4 isoform X2) c4( ) c5(282664, 282665, 282666, 282667, 282668, 282669, 282670, 282671, 282672, 282673, 282674, 282675, 282676, 282677, 282678, 282679) c6( ) c7(cDE, kC, Gq, UP); #c1(SGCA) c2(NP_000014) c3(alpha-sarcoglycan isoform 1 precursor) c4(61) c5(62, 63, 64, 65, 66, 67, 68) c6( ) c7(T, azL, aqZ, Qr, bFz, xs, bgo, oj, km, uY, oh, aan, bX, bgi, cDF, jr); #c1(SGCA) c2(NP_001129169) c3(alpha-sarcoglycan isoform 2 precursor) c4(50972, 50973) c5(50974, 50975, 50976, 50977, 50978, 50979, 50980, 50981, 50982, 50983, 50984, 50985, 50986, 50987) c6( ) c7(T, azL, aqZ, Qr, bFz, xs, bgo, oj, km, uY, oh, aan, bX, bgi, cDF, jr); #c1(SGCA) c2(XP_011523426) c3(alpha-sarcoglycan isoform X5) c4(317950) c5(317951, 317952, 317953, 317954, 317955, 317956, 317957) c6( ) c7(T, azL, aqZ, Qr, bFz, xs, bgo, oj, km, uY, oh, aan, bX, bgi, cDF, jr); #c1(SGCB) c2(NP_000223) c3(beta-sarcoglycan) c4(4781) c5(4782, 4783, 4784, 4785, 4786, 4787, 4788) c6( ) c7(T, cc, aqZ, Ae, Jl, bgu, iB, xs, qK, oj, aA, km, io, auS, af, aS, K, hB, cm, cDG); #c1(SGCB) c2(XP_01532705) c3(beta-sarcoglycan isoform X1) c4(328733) c5(328734, 328735, 328736, 328737, 328738, 328739, 328740) c6( ) c7(T, cc, aqZ, Ae, Jl, bgu, iB, xs, qK, oj, aA, km, io, auS, aF, aS, K, hB, cm, cDG); #c1(SGCD) c2(NP_758447) c3(delta-sarcoglycan isoform 2) c4(249175) c5(249176, 249177, 249178, 249179, 249180, 249181, 249182, 249183, 249184, 249185, 249186, 249187) c6( ) c7(oe, ht, Ri, cz, mw, cd, cc, lu, vc, K, agW, anR, aqZ, ff, xs, uY, en, cDH, W, ac, mx, km, cDl); #c1(SGCD) c2(XP_005266023) c3(delta-sarcoglycan isoform X1) c4(279890) c5(279891, 279892, 279893, 279894, 279895, 279896, 279897, 279898) c6( ) c7(oe, ht, Ri, cz, mw, cd, cc, lu, vc, K, agW, anR, aqZ, ff, xs, uY, en, cDH, W, ac, mx, km, cDl); #c1(SGCD) c2(XP_011532923) c3(delta-sarcoglycan isoform X2) c4(329090) c5(329091, 329092, 329093, 329094, 329095, 329096, 329097, 329098) c6( ) c7(oe, ht, Ri, cz, mw, cd, cc, lu, vc, K, agW, anR, aqZ, ff, xs, uY, en, cDH, W, ac, mx, km, cDl); #c1(SGCE) c2(NP_001092870) c3(epsilon-sarcoglycan isoform 3) c4(39661) c5(39662, 39663, 39664, 39665, 39666, 39667) c6( ) c7(qx, ha, ml, bS, ct, bd, PW, ayQ, Wk, aU, Wm, rE, he, Wi, yL, xs, LO, ckd, Z, bw, xw, bDO, dr, km, aen, qc); #c1(SGCE) c2(NP_001092871) c3(epsilon-sarcoglycan isoform 1) c4(39668) c5(39669, 39670, 39671, 39672, 39673, 39674) c6( ) c7(qx, ha, ml, bS, ct, bd, PW, ayQ, Wk, aU, Wm, rE, he, Wi, yL, xs, LO, ckd, Z, bw, xw, bDO, dr, km, aen, qc); #c1(SGCE) c2(NP_003910) c3(epsilon-sarcoglycan isoform 2) c4(142100) c5(142101, 142102, 142103, 142104, 142105, 142106) c6( ) c7(qx, ha, ml, bS, ct, bd, PW, ayQ, Wk, al, Wm, rE, he, Wi, yL, xs, LO, ckd, Z, bw, xw, bDO, dr, km, aen, qc); #c1(SGCE) c2(XP_005250732) c3(epsilon-sarcoglycan isoform X3) c4( ) c5(270494, 270495, 270496, 270497, 270498, 270499) c6( ) c7(qx, ha, ml, bS, ct, bd, PW, ayQ, Wk, aU, Wm, rE, he, Wi, yL, xs, LO, ckd, Z, bw, xw, bDO, dr, km, aen, qc); #c1(SGCE) c2(XP_011514965) c3(epsilon-sarcoglycan isoform X1) c4(307773) c5( ) c6( ) c7(qx, ha, ml, bS, ct, bd, PW, ay, Wk, aU, Wm, rE, he, Wi, yL, xs, LO, ckd, Z, bw, xw, bDO, dr, km, aen, qc); #c1(SGCG) c2(XP_005266562) c3(gamma-sarcoglycan isoform X1) c4( ) c5(280219, 280220, 280221, 280222, 280223, 280224, 280225, 280226, 280227, 280228, 280229, 280230, 280231) c6( ) c7(ng, bM, T, bB, ame, ci, aE, dE, dV, al, adB, K, bU, hH, yo, aW, lW, hl, uY, Qm, g, eF, cm, eM, wK, TG, vA, uJ, azg, m, bgi, Ri, n, BF, ct, PJ, ado, cd, b, ft, jz, cc, cU, h, jH, pi, adF, bYA, bqy, AQ, pm, op, bP, dB, rx, ER, B, bL, cz, v, j, Kh, C, q, eA, Wn, aT, yu, jj, an, yj, af, ak, wr, aqZ, s, vT, Y, r, akB, t, nC, iK, hY, ck, c, gX, km, aor, im, bKY, bt, Bh, ob, ha, auq, bS, iG, fu, Rk, aop, ds, vc, acQ, fb, SN, xX, d, xs, eH, cr, bgh, CF, yy, Pd, Z, bz, bgg, alm, bE, i, CW); #c1(SGCG) c2(XP_006719924) c3(gamma-sarcoglycan isoform X2) c4(293082) c5(293083, 293084) c6( ) c7(ng, bM, T, bB, ame, ci, aE, dE, dV, aU, adB, K, bU, hH, yo, aW, lW, hl, uY, Qm, g, eF, cm, eM, wK, TG, vA, uJ, azg, m, bgi, Ri, n, BF, ct, PJ, ado, cd, b, ft, jz, cc, cU, h, jH, pi, adF, bYA, bqy, AQ, pm, op, bP, dB, rx, ER, B, bL, cz, v, j, Kh, C, q, eA, Wn, aT, yu, jj, an, yj, af, ak, wr, aqZ, s, vT, Y, r, akB, t, nC, iK, hY, ck, c, gX, km, aor, im, bKY, bt, Bh, ob, ha, auq, bS, iG, fu, Rk, aop, ds, vc, acQ, fb, SN, xX, d, xs, eH, cr, bgh, CF, yy, Pd, Z, bz, bgg, alm, bE, i, CW); #c1(SGCZ) c2(NP_631906) c3(zeta-sarcoglycan) c4(238289, 238290) c5(238291, 238292, 238293, 238294, 238295, 238296, 238297, 238298, 238299, 238300, 238301, 238302) c6( ) c7(hb, aw, km, aj, j, Kw, an, bt, xa, ad); #c1(SGIP1) c2(XP_011540595) c3(SH3-containing GRB2-like protein 3-interacting protein 1 isoform X21) c4( ) c5(339099, 339100) c6( ) c7(ac, dY, e, aw, v, j, eu); #c1(SGK1) c2(NP_001137148) c3(serine/threonine-protein kinase Sgk1 isoform 2) c4(54991, 54992, 54993) c5(54994, 54995, 54996, 54997, 54998, 54999, 55000, 55001, 55002, 55003, 55004, 55005, 55006, 55007, 55008, 55009, 55010, 55011) c6( ) c7(bL, nD, T, jv, ahS, eR, Dd, oe, ha, aw, bO, cz, wo, n, aj, dG, v, ci, qN, fd, zP, aY, cd, J, ad, eA, py, uT, aT, iv, cy, jy, K, ae, Qe, zQ, s, aW, Y, ff, Fe, aq, dg, hp, zC, vs, en, t, cm, qa, bF, es, rR, c, eM, bE, iA, U, MM, gZ, dy, m, clP, ei, lC, bt, B, ao); #c1(SGK1) c2(NP_001137149) c3(serine/threonine-protein kinase Sgk1 isoform 3) c4(55012) c5(55013, 55014, 55015, 55016, 55017, 55018) c6( ) c7(bL, nD, T, jv, ahS, eR, Dd, oe, ha, aw, bO, cz, wo, n, aj, dG, v, ci, qN, fd, zP, aY, cd, J, ad, eA, py, uT, aT, iv, cy, jy, K, ae, Qe, zQ, s, aW, Y, ff, Fe, aq, dg, hp, zC, vs, en, t, cm, qa, bF, es, rR, c, eM, bE, iA, U, MM, gZ, dy, m, clP, ei, lC, bt, B, ao); #c1(SGK1) c2(NP_001137150) c3(serine/threonine-protein kinase Sgk1 isoform 4) c4(55019) c5(55020, 55021, 55022, 55023, 55024, 55025, 55026, 55027, 55028, 55029, 55030, 55031) c6( ) c7(bL, nD, T, jv, ahS, eR, Dd, oe, ha, aw, bO, cz, wo, n, aj, dG, v, ci, qN, fd, zP, aY, cd, J, ad, eA, py, uT, aT, iv, cy, jy, K, ae, Qe, zQ, s, aW, Y, ff, Fe, aq, dg, hp, zC, vs, en, t, cm, qa, bF, es, rR, c, eM, bE, iA, U, MM, gZ, dy, m, clP, ei, lC, bt, B, ao); #c1(SGK1) c2(NP_005618) c3(serine/threonine-protein kinase Sgk1 isoform 1) c4(157973, 157974) c5(157975, 157976, 157977, 157978, 157979, 157980) c6( ) c7(bL, nD, T, jv, ahS, eR, Dd, oe, ha, aw, bO, cz, wo, n, aj, dG, v, ci, qN, fd, zP, aY, cd, J, ad, eA, py, uT, aT, iv, cy, jy, K, ae, Qe, zQ, s, aW, Y, ff, Fe, aq, dg, hp, zC, vs, en, t, cm, qa, bF, es, rR, c, eM, bE, iA, U, MM, gZ, dy, m, clP, ei, lC, bt, B, ao); #c1(SGK2) c2(NP_001186193) c3(serine/threonine-protein kinase Sgk2 isoform alpha) c4(77376, 77377, 77378) c5(77379, 77380, 77381, 77382, 77383, 77384, 77385, 77386, 77387, 77388, 77389, 77390, 77391, 77392, 77393, 77394, 77395, 77396, 77397, 77398, 77399) c6( ) c7(K); #c1(SGK2) c2(NP_057360) c3(serine/threonine-protein kinase Sgk2 isoform beta) c4(190341, 190342) c5(190343, 190344, 190345, 190346, 190347, 190348, 190349, 190350, 190351, 190352, 190353, 190354, 190355, 190356, 190357, 190358, 190359) c6( ) c7(K); #c1(SGK223) c2(XP_005272427) c3(tyrosine-protein kinase SgK223 isoform X1) c4(284018) c5(284019, 284020, 284021, 284022, 284023, 284024, 284025, 284026, 284027, 284028) c6( ) c7(dY); #c1(SGK3) c2(NP_001028750) c3(serine/threonine-protein kinase Sgk3 isoform 1) c4(30614) c5(30615, 30616, 30617, 30618, 30619, 30620, 30621, 30622, 30623, 30624, 30625, 30626, 30627, 30628, 30629, 30630, 30631, 30632, 30633, 30634, 30635, 30636, 30637, 30638, 30639, 30640) c6( ) c7(aW, aT, K, ae, B, ad); #c1(SGK3) c2(NP_733827) c3(serine/threonine-protein kinase Sgk3 isoform 2) c4(247185) c5(247186, 247187, 247188, 247189, 247190, 247191) c6( ) c7(aW, aT, K, ae, B, ad); #c1(SGMS1) c2(XP_011537885) c3 (phosphatidylcholine:ceramide cholinephosphotransferase 1 isoform X1) c4(335383, 335384, 335385, 335386) c5(335387, 335388, 335389, 335390, 335391, 335392, 335393, 335394, 335395, 335396, 335397, 335398, 335399, 335400, 335401, 335402, 335403, 335404) c6( ) c7(ck, av, T, aC, eM, uR, r, v, ci, bz); #c1(SGMS1) c2(XP_011537887) c3(phosphatidylcholine:ceramide cholinephosphotransferase 1 isoform X3) c4( ) c5(335405, 335406, 335407, 335408, 335409, 335410) c6( ) c7(ck, av, T, aC, eM, uR, r, v, ci, bz); #c1(SGMS2) c2(NP_001129730) c3(phosphatidylcholine:ceramide cholinephosphotransferase 2) c4(52235, 52236, 52237) c5(52238, 52239, 52240, 52241, 52242, 52243, 52244, 52245, 52246, 52247, 52248, 52249, 52250, 52251, 52252, 52253, 52254, 52255, 52256, 52257, 52258, 52259, 52260, 52261, 52262, 52263) c6( ) c7(bL, dD, ea, fc, jy, v, eq, ci, cP); #c1(SGOL1) c2(NP_001012409) c3(shugoshin-like 1 isoform A1) c4(24925) c5(24926, 24927, 24928, 24929, 24930, 24931, 24932, 24933, 24934, 24935) c6( ) c7(ck, ai, T, NC, eM, ju, r, aA, aF, j, bK, bh, an, bt, bz, q); #c1(SGOL1) c2(NP_001012410) c3(shugoshin-like 1 isoform A2) c4(24936) c5(24937, 24938, 24939, 24940, 24941, 24942, 24943, 24944, 24945, 24946) c6( ) c7(ck, ai, T, NC, eM, ju, r, aA, aF, j, bK, bh, an, bt, bz, q); #c1(SGOL1) c2(NP_001012411) c3(shugoshin-like 1 isoform B1) c4(24947) c5(24948, 24949, 24950, 24951, 24952, 24953, 24954, 24955, 24956, 24957) c6( ) c7(ck, ai, T, NC, eM, ju, r, aA, aF, j, bK, bh, an, bt, bz, q); #c1(SGOL1) c2(NP_001012412) c3(shugoshin-like 1 isoform B2) c4(24958) c5(24959, 24960, 24961, 24962, 24963, 24964, 24965, 24966, 24967, 24968) c6( ) c7(ck, ai, T, NC, eM, ju, r, aA, aF, j, bK, bh, an, bt, bz, q); #c1(SGOL1) c2(NP_001012413) c3(shugoshin-like 1 isoform C1) c4(24969) c5(24970, 24971, 24972, 24973, 24974, 24975, 24976, 24977, 24978, 24979) c6( ) c7(ck, ai, T, NC, eM, ju, r, aA, aF, j, bK, bh, an, bt, bz, q); #c1(SGOL1) c2(NP_001186186) c3(shugoshin-like 1 isoform D1) c4(77321) c5(77322, 77323, 77324, 77325, 77326, 77327) c6( ) c7(ck, ai, T, NC, eM, ju, r, aA, aF, j, bK, bh, an, bt, bz, q); #c1(SGOL1) c2(NP_612493) c3(shugoshin-like 1 isoform C2) c4(236637) c5(236638, 236639, 236640, 236641, 236642, 236643, 236644, 236645, 236646, 236647) c6( ) c7(ck, ai, T, NC, eM, ju, r, aA, aF, j, bK, bh, an, bt, bz, q); #c1(SGPL1) c2(XP_011538619) c3(sphingosine-1-phosphate lyase 1 isoform X1) c4(336344, 336345) c5(336346, 336347, 336348, 336349, 336350, 336351, 336352, 336353, 336354, 336355, 336356, 336357) c6( ) c7(h, T, CM, af, fD); #c1(SGPL1) c2(XP_011538620) c3(sphingosine-1-phosphate lyase 1 isoform X2) c4(336358) c5(336359, 336360, 336361, 336362, 336363, 336364) c6( ) c7(h, T, CM, af, fD); #c1(SGPP1) c2(NP_110418) c3(sphingosine-1-phosphate phosphatase 1) c4(220192) c5(220193, 220194, 220195, 220196, 220197, 220198) c6( ) c7(T, if, fo, ci, bX, qN, Qp, V, q, eA, dP, baA, bv, iu, Xe, Qk, h, ey, K, fb, iL, bA, TC, A, nO, Y, ff, xs, eH, eZ, r, fe, uY, en, eF, nM, eM, B, fl); #c1(SGPP2) c2(NP_689599) c3(sphingosine-1-phosphate phosphatase 2) c4(243182) c5(243183, 243184, 243185, 243186, 243187, 243188) c6( ) c7(dP, cr); #c1(SGSH) c2(NP_000190) c3(N-sulphoglucosamine sulphohydrolase precursor) c4(4155, 4156) c5(4157, 4158, 4159, 4160, 4161, 4162, 4163, 4164, 4165, 4166, 4167, 4168, 4169, 4170, 4171, 4172, 4173, 4174, 4175, 4176, 4177, 4178, 4179, 4180, 4181, 4182, 4183, 4184, 4185, 4186, 4187, 4188, 4189, 4190, 4191, 4192, 4193, 4194, 4195, 4196, 4197, 4198, 4199, 4200, 4201, 4202, 4203, 4204, 4205, 4206, 4207, 4208, 4209, 4210, 4211, 4212, 4213, 4214, 4215, 4216, 4217, 4218, 4219, 4220, 4221, 4222, 4223, 4224, 4225, 4226, 4227, 4228, 4229, 4230, 4231, 4232, 4233, 4234, 4235, 4236, 4237, 4238, 4239, 4240, 4241, 4242, 4243, 4244, 4245, 4246, 4247, 4248, 4249, 4250, 4251, 4252, 4253, 4254) c6( ) c7(cDJ, lE, kg, aiR, U, Lq, agV, cbA, DL); #c1(SGSH) c2(XP_011523428) c3(N-sulphoglucosamine sulphohydrolase isoform X3) c4( ) c5(317958) c6( ) c7(cDJ, lE, kg, aiR, U, Lq, agV, cbA, DL); #c1(SGSH) c2(XP_011523429) c3(N-sulphoglucosamine sulphohydrolase isoform X4) c4(317959) c5(317960, 317961, 317962, 317963) c6( ) c7(cDJ, lE, kg, aiR, U, Lq, agV, cbA, DL); #c1(SGSM2) c2(NP_001091979) c3(small G protein signaling modulator 2 isoform 2) c4(39121) c5(39122, 39123, 39124, 39125, 39126, 39127) c6( ) c7(bL, v); #c1(SGSM2) c2(NP_055668) c3(small G protein signaling modulator 2 isoform 1) c4(182627) c5(182628, 182629, 182630, 182631, 182632, 182633) c6( ) c7(bL, v); #c1(SGSM2) c2(XP_011522412) c3(small G protein signaling modulator 2 isoform X10) c4( ) c5(317082, 317083) c6( ) c7(bL, v); #c1(SGSM3) c2(NP_056520) c3 (small G protein signaling modulator 3 isoform 1) c4(187910) c5(187911, 187912, 187913, 187914, 187915, 187916) c6( ) c7(bM, T, pf, yS, ea, if, bO, fd, bB, qj, ci, aY, GK, aE, pX, dV, Ou, K, ass, eL, cp, hm, aW, nY, hN, as, cm, yl, eM, nB, iT, vA, U, iu, dD, vl, NT, BF, qN, Hx, ft, cU, h, bl, he, A, aml, kp, pm, W, bP, hL, HB, rx, it, B, bL, DM, fM, dA, eR, aA, GC, v, j, q, er, eA, hb, Qg, aT, bK, jj, ae, af, jf, iF, Uf, NE, oV, Y, Pg, r, en, cl, f, ck, nM, WP, dy, bt, ao, hl, id, bS, dG, eq, ad, hd, py, ds, fb, nJ, d, BO, ff, eZ, Mz, gl, aF, iV, bz, dr, Hp, bF, Ew, bE, MC, y, st, pC); #c1(SGSM3) c2(XP_005261631) c3(small G protein signaling modulator 3 isoform X3) c4(277504) c5(277505) c6( ) c7(bM, T, pf, yS, ea, if, bO, fd, bB, qj, ci, aY, GK, aE, pX, dV, Ou, K, ass, eL, cp, hm, aW, nY, hN, as, cm, yl, eM, nB, iT, vA, U, iu, dD, vl, NT, BF, qN, Hx, ft, cU, h, bl, he, A, aml, kp, pm, W, bP, hL, HB, rx, it, B, bL, DM, fM, dA, eR, aA, GC, v, j, q, er, eA, hb, Qg, aT, bk, jj, ae, af, jf, iF, Uf, NE, oV, Y, Pg, r, en, cl, f, ck, nM, WP, dy, bt, ao, hl, id, bS, dG, eq, ad, hd, py, ds, fb, nJ, d, BO, ff, eZ, Mz, gl, aF, iV, bz, dr, Hp, bF, Ew, bE, MC, y, st, pC); #c1(SGSM3) c2(XP_005261632) c3(small G protein signaling modulator 3 isoform X6) c4( ) c5(277506, 277507, 277508, 277509, 277510) c6( ) c7(bM, T, pf, yS, ea, if, bO, fd, bB, qj, ci, aY, GK, aE, pX, dV, Ou, K, ass, eL, cp, hm, aW, nY, hN, as, cm, yl, eM, nB, iT, vA, U, iu, dD, vl, NT, BF, qN, Hx, ft, cU, h, bl, he, A, aml, kp, pm, W, bP, hL, HB, rx, it, B, bL, DM, fM, dA, eR, aA, GC, v, j, q, er, eA, hb, Qg, aT, bK, jj, ae, af, jf, iF, Uf, NE, oV, Y, Pg, r, en, cl, f, ck, nM, WP, dy, bt, ao, hl, id, bS, dG, eq, ad, hd, py, ds, fb, nJ, d, BO, ff, eZ, Mz, gl, aF, iV, bz, dr, Hp, bF, Ew, bE, MC, y, st, pC); #c1(SGSM3) c2(XP_005261634) c3(small G protein signaling modulator 3 isoform X7) c4(277511, 277512) c5(277513, 277514, 277515, 277516, 277517, 277518, 277519, 277520, 277521, 277522, 277523, 277524) c6( ) c7(bM, T, pf, yS, ea, if, bO, fd, bB, qj, ci, aY, GK, aE, pX, dV, Ou, K, ass, eL, cp, hm, aW, nY, hN, as, cm, yl, eM, nB, iT, vA, U, iu, dD, vl, NT, BF, qN, Hx, ft, cU, h, bl, he, A, aml, kp, pm, W, bP, hL, HB, rx, it, B, bL, DM, fM, dA, eR, aA, GC, v, j, q, er, eA, hb, Qg, aT, bK, jj, ae, af, jf, iF, Uf, NE, oV, Y, Pg, r, en, cl, f, ck, nM, WP, dy, bt, ao, hl, id, bS, dG, eq, ad, hd, py, ds, fb, nJ, d, BO, ff, eZ, Mz, gl, aF, iV, bz, dr, Hp, bF, Ew, bE, MC, y, st, pC); #c1(SGTA) c2(XP_011526480) c3(small glutamine-rich tetratricopeptide repeat-containing protein alpha isoform X1) c4(320784) c5(320785, 320786, 320787, 320788, 320789, 320790) c6( ) c7(kl, aZ, it, ae, cd, ad, Qt); #c1(SH2B1) c2(NP_001139267) c3(SH2B adapter protein 1 isoform 1) c4(59044) c5( ) c6( ) c7(bL, bwl, ae, bM, bP, gH, aw, cll, n, jy, v, j, bz, fq, ad); #c1(SH2B1) c2(NP_001139269) c3(SH2B adapter protein 1 isoform 3) c4(59045, 59046) c5(59047, 59048, 59049, 59050, 59051, 59052, 59053, 59054, 59055, 59056, 59057, 59058) c6( ) c7(bL, bwl, ae, bM, bP, gH, aw, cll, n, jy, v, j, bz, fq, ad); #c1(SH2B1) c2(NP_056318) c3(SH2B adapter protein 1 isoform 2) c4(187042, 187043) c5(187044, 187045, 187046, 187047, 187048, 187049, 187050, 187051, 187052, 187053, 187054, 187055, 187056, 187057, 187058, 187059, 187060) c6( ) c7(bL, bwl, ae, bM, bP, gH, aw, cll, n, jy, v, j, bz, fq, ad); #c1(SH2B2) c2(NP_066189) c3(SH2B adapter protein 2) c4(206818) c5(206819, 206820, 206821, 206822, 206823, 206824, 206825, 206826, 206827, 206828, 206829, 206830) c6( ) c7(T, il, ag, eZ, ez, fF, OS, dX); #c1(SH2B2) c2(XP_005277032) c3(SH2B adapter protein 2 isoform X1) c4( ) c5(285748) c6( ) c7(T, il, ag, eZ, ez, fF, OS, dX); #c1(SH2B2) c2(XP_011514042) c3(SH2B adapter protein 2 isoform X3) c4(306475, 306476) c5( ) c6( ) c7(T, il, ag, eZ, ez, fF, OS, dX); #c1(SH2B2) c2(XP_011514043) c3(SH2B adapter protein 2 isoform X4) c4(306477, 306478) c5(306479, 306480, 306481, 306482, 306483, 306484, 306485, 306486, 306487, 306488, 306489, 306490, 306491, 306492, 306493, 306494, 306495, 306496) c6( ) c7(T, il, ag, eZ, ez, fF, OS, dX); #c1(SH2B3) c2(NP_001278353) c3(SH2B adapter protein 3 isoform 2) c4(109591) c5(109592, 109593, 109594, 109595, 109596, 109597) c6( ) c7(oO, bM, T, oe, yG, lo, e, cz, ez, hz, cL, aw, j, qj, bh, V, o, x, pN, dz, K, hK, A, et, eZ, hp, pm, ck, ac, c, pc, pg, aS, k, gT, y); #c1(SH2B3) c2(NP_005466) c3(SH2B adapter protein 3 isoform 1) c4(156412) c5(156413, 156414, 156415, 156416, 156417, 156418) c6( ) c7(oO, bM, T, oe, yG, lo, e, cz, ez, hz, cL, aw, j, qj, bh, V, o, x, pN, dz, K, hK, A, et, eZ, hp, pm, ck, ac, c, pc, pg, aS, k, gT, y); #c1(SH2B3) c2(XP_006719243) c3(SH2B adapter protein 3 isoform X6) c4( ) c5(292355) c6( ) c7(oO, bM, T, oe, yG, lo, e, cz, ez, hz, cL, aw, j, qj, bh, V, o, x, pN, dz, K, hK, A, et, eZ, hp, pm, ck, ac, c, pc, pg, aS, k, gT, y); #c1(SH2D1A) c2(NP_001108409) c3(SH2 domain-containing protein (A isoform 2) c4(43187) c5(43188, 43189, 43190, 43191, 43192, 43193, 43194, 43195, 43196, 43197, 43198, 43199) c6( ) c7(T, fM, dA, fR, iW, aw, eX, jh, aj, v, C, pz, Za, aR, aGT, J, hX, F, aCF, ds, kH, hD, h, eV, Ze, iY, H, amA, Zd, pk, hm, A, YY, oV, Zf, eZ, lo, ho, Zb, mK, dr, Wt, Cv, ck, L, cDK, xD, Zg, dW, CZ, yH, R, bC, y, bNP, ZA, Ro); #c1(SH2D1A) c2(NP_002342) c3(SH2 domain-containing protein 1A isoform 1) c4(127682, 127683) c5(127684, 127685, 127686, 127687, 127688, 127689) c6( ) c7(T, fM, dA, fR, iW, aw, eX, jh, aj, v, C, pz, Za, aR, aGT, J, hX, F, aCF, ds, kH, hD, h, eV, Ze, iY, H, amA, Zd, pk, hm, A, YY, oV, Zf, eZ, lo, ho, Zb, mK, dr, Wt, Cv, ck, L, cDK, xD, Zg, dW, CZ, yH, R, bC, y, bNP, zA, Ro); #c1(SH2D2A) c2(NP_001154913) c3(SH2 domain-containing protein 2A isoform 1) c4(61227) c5(61228, 61229, 61230, 61231, 61232, 61233) c6( ) c7(yW, aw, aFT, cL, j, y, V, o); #c1(SH2D2A) c2(NP_001154914) c3(SH2 domain-containing protein ZA isoform 3) c4(61234) c5(61235, 61236, 61237, 61238, 61239, 61240) c6( ) c7(yW, aw, aFT, cL, j, y, V, o); #c1(SH2D2A) c2(NP_003966) c3(SH2 domain-containing protein 2A isoform 2) c4(142502) c5(142503, 142504, 142505, 142506, 142507, 142508) c6( ) c7(yW, aw, aFT, cL, j, y, V, o); #c1(SH2D3A) c2(NP_005481) c3(SH2 domain-containing protein 3A) c4(156581) c5(156582, 156583, 156584, 156585, 156586, 156587) c6( ) c7(lo, wc, awi, aT); #c1(SH2D3A) c2(XP_005259529) c3(SH2 domain-containing protein 3A isoform X4) c4(276114) c5(276115, 276116, 276117, 276118, 276119, 276120) c6( ) c7(lo, wc, awi, aT); #c1(SH2D3A) c2(XP_006722665) c3(SH2 domain-containing protein 3A isoform X3) c4( ) c5(294795, 294796) c6( ) c7(lo, wc, awi, aT); #c1(SH2D3C) c2(NP_001136005) c3(SH2 domain-containing protein 3C isoform d) c4(53967) c5(53968, 53969, 53970, 53971, 53972, 53973) c6( ) c7(Na, zT, xA, BD, aW, wc, aT, Nc, zW, cm); #c1(SH2D3C) c2(NP_001136006) c3(SH2 domain-containing protein 3C isoform e) c4(53974) c5(53975, 53976, 53977, 53978, 53979, 53980, 53981, 53982, 53983, 53984, 53985) c6( ) c7(Na, zT, xA, BD, aW, wc, aT, Nc, zW, cm); #c1(SH2D3C) c2(NP_001239263) c3(SH2 domain-containing protein 3C isoform f) c4(88391) c5(88392, 88393, 88394, 88395, 88396, 88397) c6( ) c7(Na, zT, xA, BD, aW, wc, aT, Nc, zW, cm); #c1(SH2D3C) c2(NP_005480) c3(SH2 domain-containing protein 3C isoform b) c4(156567) c5(156568, 156569, 156570, 156571, 156572, 156573, 156574, 156575, 156576, 156577, 156578, 156579, 156580) c6( ) c7(Na, zT, xA, BD, aW, wc, aT, Nc, zW, cm); #c1(SH2D3C) c2(NP_733745) c3(SH2 domain-containing protein 3C isoform a) c4(246902, 246903, 246904, 246905) c5(246906, 246907, 246908, 246909, 246910, 246911, 246912, 246913, 246914, 246915, 246916, 246917) c6( ) c7(Na, zT, xA, BD, aW, wc, aT, Nc, zW, cm); #c1(SH2D4A) c2(NP_001167630) c3(SH2 domain-containing protein 4A isoform a) c4(69471) c5(69472, 69473, 69474, 69475, 69476, 69477, 69478, 69479, 69480, 69481) c6( ) c7(B, j); #c1(SH2D4A) c2(NP_001167631) c3(SH2 domain-containing protein 4A isoform b) c4(69482) c5(69483, 69484, 69485, 69486, 69487, 69488) c6( ) c7(B, j); #c1(SH2D4B) c2(NP_001139191) c3(SH2 domain-containing protein 48 isoform 2) c4(58913) c5(58914, 58915, 58916, 58917, 58918, 58919) c6( ) c7(bqW); #c1(SH2D4B) c2(NP_997255) c3(SH2 domain-containing protein 48 isoform 1) c4(265332, 265333, 265334) c5(265335, 265336, 265337, 265338, 265339, 265340, 265341, 265342, 265343, 265344, 265345, 265346, 265347, 265348, 265349, 265350, 265351, 265352) c6( ) c7(bqW); #c1(SH3BGR) c2(NP_001001713) c3(SH3 domain-binding glutamic acid-rich protein isoform b) c4( ) c5(18973, 18974, 18975, 18976, 18977, 18978, 18979, 18980, 18981, 18982, 18983, 18984, 18985, 18986, 18987, 18988) c6( ) c7(f, v, j); #c1(SH3BGR) c2(NP_031367) c3(SH3 domain-binding glutamic acid-rich protein isoform a) c4(171965) c5( ) c6( ) c7(f, v, j); #c1(SH3BGR) c2(XP_011527990) c3(SH3 domain-binding glutamic acid-rich protein isoform X2) c4( ) c5(323043, 323044, 323045, 323046, 323047, 323048, 323049, 323050, 323051) c6( ) c7(f, v, j); #c1(SH3BGRL) c2(NP_003013) c3(SH3 domain-binding glutamic acid-rich-like protein) c4(134432) c5(134433, 134434, 134435, 134436, 134437, 134438) c6( ) c7(f, v, j); #c1(SH3BGRL2) c2(NP_113657) c3(SH3 domain-binding glutamic acid-rich-like protein 2) c4(221975) c5(221976, 221977, 221978, 221979, 221980, 221981) c6( ) c7(nX, lr); #c1(SH3BP1) c2(NP_061830) c3(SH3 domain-binding protein 1) c4(200645, 200646, 200647, 200648) c5(200649, 200650, 200651, 200652, 200653, 200654, 200655, 200656, 200657, 200658, 200659, 200660, 200661, 200662, 200663, 200664, 200665, 200666, 200667, 200668, 200669, 200670, 200671, 200672) c6( ) c7(aj, bo); #c1(SH3BP2) c2(NP_001139327) c3(SH3 domain-binding protein 2 isoform c) c4(59194, 59195, 59196) c5(59197, 59198, 59199, 59200, 59201, 59202, 59203, 59204) c6( ) c7(ck, azi, fr, cDL, zu, dr); #c1(SH3BP2) c2(XP_005248055) c3(SH3 domain-binding protein 2 isoform X4) c4(268182) c5(268183) c6( ) c7(ck, azi, fr, cDL, zu, dr); #c1(SH3BP2) c2(XP_011511849) c3(SH3 domain-binding protein 2 isoform X2) c4(302521) c5(302522, 302523, 302524, 302525, 302526, 302527, 302528) c6( ) c7(ck, azi, fr, cDL, zu, dr); #c1(SH3BP2) c2(XP_011511850) c3(SH3 domain-binding protein 2 isoform X3) c4(302529, 302530, 302531) c5( ) c6( ) c7(ck, azi, fr, cDL, zu, dr); #c1(SH3BP2) c2(XP_011511852) c3(SH3 domain-binding protein 2 isoform X1) c4( ) c5(302532, 302533, 302534, 302535, 302536, 302537, 302538, 302539, 302540, 302541, 302542, 302543, 302544, 302545) c6( ) c7(ck, azi, fr, cDL, zu, dr); #c1(SH3BP2) c2(XP_011511853) c3(SH3 domain-binding protein 2 isoform X5) c4(302546) c5( ) c6( ) c7(ck, azi, fr, cDL, zu, dr); #c1(SH3BP2) c2(XP_011511858) c3(SH3 domain-binding protein 2 isoform X10) c4( ) c5(302547, 302548, 302549, 302550, 302551, 302552, 302553, 302554, 302555, 302556, 302557, 302558, 302559, 302560, 302561, 302562, 302563, 302564, 302565, 302566, 302567, 302568, 302569, 302570, 302571, 302572, 302573, 302574, 302575, 302576, 302577, 302578, 302579, 302580, 302581, 302582, 302583, 302584, 302585, 302586) c6( ) c7(ck, azi, fr, cDL, zu, dr); #c1(SH3BP4) c2(XP_011509196) c3(SH3 domain-binding protein 4 isoform X1) c4(299809, 299810, 299811, 299812, 299813, 299814, 299815) c5(299816, 299817, 299818, 299819, 299820, 299821, 299822, 299823, 299824, 299825, 299826, 299827, 299828, 299829, 299830, 299831, 299832, 299833, 299834, 299835, 299836, 299837, 299838, 299839, 299840, 299841, 299842, 299843, 299844, 299845, 299846, 299847, 299848, 299849, 299850, 299851, 299852, 299853, 299854, 299855, 299856, 299857, 299858, 299859, 299860, 299861, 299862, 299863, 299864, 299865) c6( ) c7(bL, T, DM, eR, oe, DV, bS, v, ai, Na, ql, aT, h, DO, DG, jj, s, aW, ev, aq, iR, iK, ck, DT, ei, KS, B); #c1(SH3BP5) c2(NP_001018009) c3(SH3 domain-binding protein 5 isoform b) c4(26802, 26803, 26804) c5(26805, 26806, 26807, 26808, 26809, 26810, 26811, 26812, 26813, 26814, 26815, 26816, 26817, 26818, 26819, 26820, 26821, 26822, 26823, 26824, 26825) c6( ) c7(hb, azi, aw, af); #c1(SH3BP5) c2(NP_004835) c3(SH3 domain-binding protein 5 isoform a) c4(150166) c5(150167, 150168, 150169, 150170, 150171, 150172, 150173) c6( ) c7(hb, azi, aw, af); #c1(SH3BP5) c2(XP_011532554) c3(SH3 domain-binding protein 5 isoform X3) c4( ) c5(328648, 328649) c6( ) c7(hb, azi, aw, af); #c1(SH3BP5) c2(XP_011532555) c3(SH3 domain-binding protein 5 isoform X4) c4( ) c5(328650, 328651) c6( ) c7(hb, azi, aw, af); #c1(SH3D19) c2(NP_001009555) c3(SH3 domain-containing protein 19 isoform 1) c4( ) c5(23596, 23597, 23598, 23599, 23600, 23601, 23602, 23603) c6( ) c7(bL, gx, T, bM, jh, cz, bB, v, afE, q, br, ad, hX, x, ke, hd, dV, aT, h, jy, hn, dL, K, aAt, bl, jj, nJ, ae, eL, eG, d, iU, aW, Y, oV, fc, r, aq, aF, Pd, cm, bw, bz, Qt, qa, ek, QA, ck, nM, iT, bE, arx, k, cP, y, bt, B); #c1(SH3D19) c2(NP_001122395) c3(SH3 domain-containing protein 19 isoform 2) c4(46643) c5(46644, 46645, 46646, 46647, 46648, 46649, 46650, 46651, 46652, 46653, 46654) c6( ) c7(bL, gx, T, bM, jh, cz, bB, v, afE, q, br, ad, hX, x, ke, hd, dV, aT, h, jy, hn, dL, K, aAt, bl, jj, nJ, ae, eL, eG, d, iU, aW, Y, oV, fc, r, aq, aF, Pd, cm, bw, bz, Qt, qa, ek, QA, ck, nM, iT, bE, arx, k, cP, y, bt, B); #c1(SH3D19) c2(NP_001122396) c3(SH3 domain-containing protein 19 isoform 3) c4(46655) c5(46656, 46657, 46658, 46659, 46660, 46661, 46662, 46663) c6( ) c7(bL, gx, T, bM, jh, cz, bB, v, afE, q, br, ad, hX, x, ke, hd, dV, aT, h, jy, hn, dL, K, aAt, bl, jj, nJ, ae, eL, eG, d, iU, aW, Y, oV, fc, r, aq, aF, Pd, cm, bw, bz, Qt, qa, ek, QA, ck, nM, iT, bE, arx, k, cP, y, bt, B); #c1(SH3D19) c2(XP_011529950) c3(SH3 domain-containing protein 19 isoform X4) c4(325743) c5(325744, 325745, 325746, 325747, 325748, 325749, 325750) c6( ) c7(bL, gx, T, bM, jh, cz, bB, v, afE, q, br, ad, hX, x, ke, hd, dV, aT, h, jy, hn, dL, K, aAt, bl, jj, nJ, ae, eL, eG, d, iU, aW, Y, oV, fc, r, aq, af, Pd, cm, bw, bz, Qt, qa, ek, QA, ck, nM, lT, bE, arx, k, cP, y, bt, B); #c1(SH3GL1) c2(NP_001186872) c3(endophilin-A2 isoform 2) c4(78728) c5(78729, 78730, 78731, 78732, 78733, 78734) c6( ) c7(ck, x, aT, bw, k, hF, bt, fs, eA); #c1(SH3GL1) c2(NP_001186873) c3(endophilin-A2 isoform 3) c4(78735) c5(78736, 78737, 78738, 78739, 78740, 78741) c6( ) c7(ck, x, aT, bw, k, hF, bt, fs, eA); #c1(SH3GL1) c2(NP_003016) c3(endophilin-A2 isoform 1) c4(134467) c5(134468, 134469, 134470, 134471, 134472, 134473) c6( ) c7(ck, x, aT, bw, k, hF, bt, fs, eA); #c1(SH3GL2) c2(NP_003017) c3(endophilin-A1) c4(134474, 134475, 134476, 134477) c5(134478, 134479, 134480, 134481, 134482, 134483, 134484, 134485, 134486, 134487, 134488, 134489, 134490) c6( ) c7(T, AW, d, id, af, eM, aW, iK, al, vr, Ej, K, an, V, cd); #c1(SH3GL2) c2(XP_005251606) c3(endophilin-A1 isoform X2) c4( ) c5(271005, 271006, 271007, 271008, 271009, 271010, 271011, 271012, 271013, 271014, 271015) c6( ) c7(T, AW, d, id, af, eM, aW, ik, aT, vr, Ej, K, an, V, cd); #c1(SH3GL3) c2(NP_001288038) c3(endophilin-A3 isoform c) c4(113439) c5(113440, 113441, 113442, 113443, 113444, 113445, 113446, 113447, 113448, 113449) c6( ) c7(eA); #c1(SH3GL3) c2(NP_003018) c3(endophilin-A3 isoform a) c4(134491, 134492) c5(134493, 134494, 134495, 134496, 134497, 134498, 134499, 134500, 134501, 134502, 134503, 134504) c6( ) c7(eA); #c1(SH3KBP1) c2(NP_001019837) c3(SH3 domain-containing kinase-binding protein 1 isoform b) c4(27937, 27938, 27939, 27940) c5(27941, 27942, 27943, 27944, 27945, 27946, 27947, 27948, 27949, 27950, 27951, 27952, 27953, 27954, 27955, 27956, 27957, 27958, 27959, 27960, 27961, 27962, 27963, 27964) c6( ) c7(eW, T, fR, aW, ff, aT, iv, en, X); #c1(SH3KBP1) c2(NP_001171889) c3(SH3 domain-containing kinase-binding protein 1 isoform c) c4(71966) c5(71967, 71968, 71969, 71970, 71971, 71972, 71973, 71974, 71975, 71976, 71977, 71978) c6( ) c7(eW, T, fR, aW, ff, aT, iv, en, X); #c1(SH3KBP1) c2(NP_114098) c3(SH3 domain-containing kinase-binding protein 1 isoform a) c4(222155) c5(222156, 222157, 222158, 222159, 222160, 222161) c6( ) c7(eW, T, FR, aW, ff, aT, iv, en, X); #c1(SH3KBP1) c2(XP_011543800) c3(SH3 domain-containing kinase-binding protein 1 isoform X1) c4(342922) c5(342923) c6( ) c7(eW, T, fR, aW, ff, al, iv, en, X); #c1(SH3KBP1) c2(XP_011543801) c3(SH3 domain-containing kinase-binding protein 1 isoform X3) c4(342924, 342925) c5(342926, 342927, 342928, 342929, 342930, 342931, 342932, 342933, 342934, 342935, 342936) c6( ) c7(eW, T, fR, aW, ff, aT, iv, en, X); #c1(SH3KBP1) c2(XP_011543803) c3(SH3 domain-containing kinase-binding protein 1 isoform X5) c4( ) c5(342937, 342938, 342939, 342940, 342941, 342942) c6( ) c7(eW, T, FR, aW, ff, aT, iv, en, X); #c1(SH3KBP1) c2(XP_011543804) c3(SH3 domain-containing kinase-binding protein 1 isoform X6) c4(342943) c5( ) c6( ) c7(eW, T, fR, aW, ff, aT, iv, en, X); #c1(SH3PXD2A) c2(NP_055446) c3(SH3 and PX domain-containing protein 2A) c4(181192, 181193, 181194) c5(181195, 181196, 181197, 181198, 181199, 181200, 181201, 181202, 181203, 181204, 181205, 181206, 181207, 181208, 181209, 181210, 181211, 181212) c6( ) c7(iu, By, af, eb); #c1(SH3PXD2A) c2(XP_005270351) c3(SH3 and PX domain-containing protein 2A isoform X3) c4(283362) c5(283363, 283364, 283365, 283366, 283367, 283368) c6( ) c7(iu, By, af, eb); #c1(SH3PXD2A) c2(XP_005270352) c3(SH3 and PX domain-containing protein ZA isoform X4) c4(283369, 283370) c5(283371, 283372, 283373, 283374, 283375, 283376, 283377, 283378, 283379, 283380, 283381, 283382, 283383) c6( ) c7(iu, By, af, eb); #c1(SH3PXD2A) c2(XP_005270354) c3(SH3 and PX domain-containing protein ZA isoform X6) c4(283384) c5(283385, 283386, 283387, 283388, 283389, 283390) c6( ) c7(iu, By, af, eb); #c1(SH3PXD2A) c2(XP_005270355) c3(SH3 and PX domain-containing protein 2A isoform X8) c4(283391) c5(283392, 283393, 283394, 283395, 283396) c6( ) c7(iu, By, af, eb); #c1(SH3PXD2B) c2(NP_001017995) c3(SH3 and PX domain-containing protein 2B) c4(26746) c5(26747, 26748, 26749, 26750, 26751, 26752) c6( ) c7(aBU, bE, cDN, Az, bB, v, cDM, bD, xb, zo); #c1(SH3RF1) c2(NP_065921) c3(E3 ubiquitin-protein ligase SH3RF1) c4(206258, 206259, 206260) c5(206261, 206262, 206263, 206264, 206265, 206266, 206267, 206268, 206269, 206270, 206271, 206272, 206273, 206274, 206275, 206276, 206277, 206278) c6( ) c7(bz, an, aT); #c1(SH3RF3) c2(NP_001092759) c3(SH3 domain-containing RING finger protein 3 precursor) c4(39621, 39622) c5(39623, 39624, 39625, 39626, 39627, 39628) c6( ) c7(xa); #c1(SH3RF3) c2(XP_011509411) c3(SH3 domain-containing RING finger protein 3 isoform X1) c4(299968) c5(299969, 299970, 299971, 299972, 299973, 299974) c6( ) c7(xa); #c1(SH3RF3) c2(XP_011509412) c3(SH3 domain-containing RING finger protein 3 isoform X3) c4( ) c5(299975, 299976, 299977, 299978, 299979, 299980) c6( ) c7(xa); #c1(SH3TC2) c2(NP_078853) c3(SH3 domain and tetratricopeptide repeat-containing protein 2) c4(215759, 215760, 215761, 215762) c5(215763, 215764, 215765, 215766, 215767, 215768, 215769, 215770, 215771, 215772, 215773, 215774, 215775, 215776, 215777, 215778, 215779, 215780, 215781, 215782, 215783, 215784, 215785, 215786) c6( ) c7(Js, lq, agU, aFT, bQ, cDO, cDP, fu, bu, Jo, bN); #c1(SH3YL1) c2(NP_001153069) c3(SH3 domain-containing YSC84-like protein 1 isoform 2) c4(60408) c5(60409, 60410, 60411, 60412, 60413, 60414, 60415, 60416, 60417, 60418, 60419, 60420) c6( ) c7(cm); #c1(SH3YL1) c2(NP_001269611) c3(SH3 domain-containing YSC84-like protein 1 isoform 3) c4(102201) c5(102202, 102203, 102204, 102205, 102206, 102207, 102208, 102209, 102210, 102211) c6( ) c7(cm); #c1(SH3YL1) c2(NP_001269616) c3(SH3 domain-containing YSC84-like protein 1 isoform 4) c4(102213) c5(102214, 102215, 102216, 102217, 102218, 102219) c6( ) c7(cm); #c1(SH3YL1) c2(NP_056492) c3(SH3 domain-containing YSC84-like protein 1 isoform 1) c4(187743, 187744) c5(187745, 187746, 187747, 187748, 187749, 187750, 187751, 187752, 187753, 187754) c6( ) c7(cm); #c1(SHANK1) c2(XP_006723296) c3(SH3 and multiple ankyrin repeat domains protein 1 isoform X1) c4(295100, 295101) c5(295102, 295103, 295104, 295105, 295106, 295107) c6( ) c7(aQc, Pi, gl, fr, nA, rz, bN, hq); #c1(SHANK1) c2(XP_011525315) c3(SH3 and multiple ankyrin repeat domains protein 1 isoform X2) c4(319778) c5(319779, 319780, 319781, 319782, 319783, 319784) c6( ) c7(aQc, Pi, gl, fr, nA, rz, bN, hq); #c1(SHANK1) c2(XP_011525316) c3(SH3 and multiple ankyrin repeat domains protein 1 isoform X3) c4(319785) c5(319786, 319787, 319788, 319789, 319790, 319791, 319792, 319793, 319794, 319795, 319796, 319797, 319798, 319799, 319800) c6( ) c7(aQc, Pi, gl, fr, nA, rz, bN, hq); #c1(SHANK2) c2(NP_036441) c3(SH3 and multiple ankyrin repeat domains protein 2 isoform 1) c4(173899, 173900, 173901, 173902) c5(173903, 173904, 173905, 173906, 173907, 173908, 173909, 173910, 173911, 173912, 173913, 173914, 173915, 173916, 173917, 173918, 173919, 173920) c6( ) c7(rz, oe, ff, VT, it, Ea, nA, Wl, cDQ, en, aHr, aR, bN); #c1(SHANK2) c2(NP_573573) c3(SH3 and multiple ankyrin repeat domains protein 2 isoform 2) c4(234574, 234575, 234576, 234577, 234578) c5(234579, 234580, 234581, 234582, 234583, 234584, 234585, 234586, 234587, 234588, 234589, 234590, 234591, 234592, 234593, 234594, 234595, 234596, 234597, 234598, 234599, 234600, 234601, 234602, 234603, 234604, 234605, 234606, 234607, 234608) c6( ) c7(rz, oe, ff, VT, it, Ea, nA, Wl, cDQ, en, aHr, aR, bN); #c1(SHANK2) c2(XP_005277989) c3(SH3 and multiple ankyrin repeat domains protein 2 isoform X9) c4(285764) c5(285765, 285766, 285767, 285768, 285769, 285770) c6( ) c7(rz, oe, ff, VT, it, Ea, nA, Wl, cDQ, en, aHr, aR, bN); #c1(SHANK2) c2(XP_011543160) c3(SH3 and multiple ankyrin repeat domains protein 2 isoform X7) c4( ) c5(341989, 341990, 341991, 341992, 341993, 341994) c6( ) c7(rz, oe, ff, VT, it, Ea, nA, Wl, cDQ, en, aHr, aR, bN); #c1(SHANK3) c2(NP_277052) c3(SH3 and multiple ankyrin repeat domains protein 3) c4(228822, 228823) c5(228824, 228825, 228826, 228827, 228828, 228829, 228830, 228831, 228832, 228833, 228834, 228835) c6( ) c7(bcq, VX, rz, cDS, cDR, aHr, VT, acg, akf, Ko, aQM, nA, bSk, Wl, he, ahg, cnv, ad, bN); #c1(SHARPIN) c2(NP_112236) c3(sharpin) c4(221057, 221058) c5(221059, 221060, 221061, 221062, 221063, 221064, 221065, 221066, 221067, 221068, 221069, 221070, 221071) c6( ) c7(yQ, aT, dN, Xm, cbL, eE, ae, eA); #c1(SHB) c2(NP_003019) c3(SH2 domain-containing adapter protein B) c4(134505, 134506) c5(134507, 134508, 134509, 134510, 134511, 134512, 134513, 134514, 134515, 134516, 134517, 134518, 134519) c6( ) c7(hb, af, cd, ac, c); #c1(SHBG) c2(NP_001139751) c3(sex hormone-binding globulin isoform 2 precursor) c4(59844, 59845) c5(59846, 59847, 59848, 59849, 59850, 59851) c6( ) c7(T, ea, e, al, ci, aTS, aex, il, iX, auB, K, eL, xQ, aW, fc, Ku, g, AM, vA, Gs, m, pw, Uw, dD, gU, sw, zX, aj, ct, ml, qN, jw, PD, UD, jy, dL, afV, uV, hB, wC, ael, Nr, sq, hQ, aq, po, op, iO, se, afc, JQ, Pl, mg, hL, at, it, B, Rf, bL, aA, cz, ti, v, j, q, J, UE, aT, ae, af, bA, qw, kl, ND, Lf, r, bu, cl, t, Px, f, bN, vK, sn, hr, UF, gT, aZ, iW, RH, jh, btu, bS, eq, dG, ad, yt, vJ, io, aeQ, eZ, yy, dq, aF, Z, Nq, pv, yE, z, gZ, cP, ei, y); #c1(SHBG) c2(NP_001139752) c3(sex hormone-binding globulin isoform 3 precursor) c4(59852, 59853, 59854) c5(59855, 59856, 59857, 59858, 59859, 59860, 59861, 59862, 59863, 59864, 59865, 59866, 59867) c6( ) c7(T, ea, e, al, ci, aTS, aex, il, iX, auB, K, eL, xQ, aW, fc, Ku, g, AM, vA, Gs, m, pw, Uw, dD, gU, sw, zX, aj, ct, ml, qN, jw, PD, UD, jy, dL, afV, uV, hB, wC, ael, Nr, sq, hQ, aq, po, op, iO, se, afc, JQ, Pl, mg, hL, at, it, B, Rf, bL, aA, cz, ti, v, j, q, J, UE, aT, ae, af, bA, qw, kl, ND, Lf, r, bu, cl, t, Px, f, bN, vK, sn, hr, UF, gT, aZ, iW, RH, jh, btu, bS, eq, dG, ad, yt, vJ, io, aeQ, eZ, yy, dq, aF, Z, Nq, pv, yE, z, gZ, cP, ei, y); #c1(SHBG) c2(NP_001139753) c3(sex hormone-binding globulin isoform 4 precursor) c4(59868) c5(59869, 59870, 59871, 59872, 59873, 59874, 59875, 59876, 59877, 59878, 59879, 59880) c6( ) c7(T, ea, e, al, ci, aTS, aex, il, iX, auB, K, eL, xQ, aW, fc, Ku, g, AM, vA, Gs, m, pw, Uw, dD, gU, sw, zX, aj, ct, ml, qN, jw, PD, UD, jy, dL, afV, uV, hB, wC, ael, Nr, sq, hQ, aq, po, op, iO, se, afc, JQ, Pl, mg, hL, at, it, B, Rf, bL, aA, cz, ti, v, j, q, J, UE, aT, ae, af, bA, qw, kl, ND, Lf, r, bu, cl, t, Px, f, bN, vK, sn, hr, UF, gT, aZ, iW, RH, jh, btu, bS, eq, dG, ad, yt, vJ, io, aeQ, eZ, yy, dq, aF, Z, Nq, pv, yE, z, gZ, cP, ei, y); #c1(SHBG) c2(NP_001276044) c3(sex hormone-binding globulin isoform 6) c4( ) c5(107831, 107832, 107833, 107834, 107835) c6( ) c7(T, ea, e, al, ci, aTS, aex, il, iX, auB, K, eL, xQ, aW, fc, Ku, g, AM, vA, Gs, m, pw, Uw, dD, gU, sw, zX, aj, ct, ml, qN, jw, PD, UD, jy, dL, afV, uV, hB, wC, ael, Nr, sq, hQ, aq, po, op, iO, se, afc, JQ, Pl, mg, hL, at, it, B, Rf, bL, aA, cz, ti, v, j, q, J, UE, aT, ae, af, bA, qw, kl, ND, Lf, r, bu, cl, t, Px, f, bN, vK, sn, hr, UF, gT, aZ, iW, RH, jh, btu, bS, eq, dG, ad, yt, vJ, io, aeQ, eZ, yy, dq, aF, Z, Nq, pv, yE, z, gZ, cP, ei, y); #c1(SHC1) c2(NP_001123512) c3(SHC-transforming protein 1 isoform 3) c4(47267, 47268, 47269) c5(47270, 47271, 47272, 47273, 47274, 47275, 47276, 47277, 47278, 47279, 47280, 47281, 47282, 47283, 47284, 47285, 47286) c6( ) c7(bL, T, eR, e, aj, ci, PJ, J, ad, dP, aT, h, K, dQ, ae, d, B, aW, Y, Pg, aLJ, aq, bz, X, vi); #c1(SHC1) c2(NP_001123513) c3(SHC-transforming protein 1 isoform 4) c4( ) c5(47287, 47288, 47289, 47290, 47291, 47292, 47293) c6( ) c7(bL, T, eR, e, aj, ci, PJ, J, ad, dP, aT, h, K, dQ, ae, d, B, aW, Y, Pg, aLJ, aq, bz, X, vi); #c1(SHC1) c2(NP_001189788) c3(SHC-transforming protein 1 isoform 5 precursor) c4(80269, 80270, 80271) c5(80272, 80273, 80274, 80275, 80276, 80277, 80278, 80279, 80280, 80281, 80282, 80283, 80284, 80285, 80286, 80287, 80288, 80289, 80290, 80291, 80292, 80293, 80294) c6( ) c7(bL, T, eR, e, aj, ci, PJ, J, ad, dP, aT, h, K, dQ, ae, d, B, aW, Y, Pg, aLJ, aq, bz, X, vi); #c1(SHC1) c2(NP_003020) c3(SHC-transforming protein 1 isoform 2) c4(134520) c5(134521) c6( ) c7(bL, T, eR, e, aj, ci, PJ, J, ad, dP, aT, h, K, dQ, ae, d, B, aW, Y, Pg, aLJ, aq, bz, X, vi); #c1(SHC1) c2(NP_892113) c3(SHC-transforming protein 1 isoform 1) c4(257693, 257694) c5(257695, 257696, 257697, 257698, 257699, 257700) c6( ) c7(bL, T, eR, e, aj, ci, PJ, J, ad, dP, aT, h, K, dQ, ae, d, B, aW, Y, Pg, aLJ, aq, bz, X, vi); #c1(SHC1) c2(XP_011508194) c3(SHC-transforming protein 1 isoform X2) c4(298782) c5( ) c6( ) c7(bL, T, eR, e, aj, ci, PJ, J, ad, dP, aT, h, K, dQ, ae, d, B, aW, Y, Pg, al J, aq, bz, X, vi); #c1(SHC1) c2(XP_011508196) c3(SHC-transforming protein 1 isoform X4) c4(298783) c5( ) c6( ) c7(bL, T, eR, e, aj, ci, PJ, J, ad, dP, aT, h, K, dQ, ae, d, B, aW, Y, Pg, aLJ, aq, bz, X, vi); #c1(SHC1) c2(XP_011508198) c3(SHC-transforming protein 1 isoform X6) c4( ) c5(298784) c6( ) c7(bL, T, eR, e, aj, ci, PJ, J, ad, dP, aT, h, K, dQ, ae, d, B, aW, Y, Pg, aLJ, aq, bz, X, vi); #c1(SHC1) c2(XP_011508199) c3(SHC-transforming protein 1 isoform X8) c4(298785) c5(298786, 298787, 298788, 298789, 298790, 298791, 298792, 298793, 298794, 298795, 298796, 298797, 298798, 298799, 298800, 298801, 298802) c6( ) c7(bL, T, eR, e, aj, ci, PJ, J, ad, dP, aT, h, K, dQ, ae, d, B, aW, Y, Pg, aLJ, aq, bz, X, vi); #c1(SHC2) c2(NP_036567) c3(SHC-transforming protein 2) c4(174449) c5(174450, 174451, 174452, 174453, 174454, (74455) c6( ) c7(GC, Mb, yA, aha); #c1(SHC3) c2(NP_058544) c3(SHC-transforming protein 3) c4(192818) c5(192819, 192820, 192821, 192822, 192823, 192824, 192825) c6( ) c7(iC, T, sS, d, hd, nB, aW, Lf, XY, cj, aT, bnt, ci, as, bu, cm, bz, eA, zn); #c1(SHC3) c2(XP_011517087) c3(SHC-transforming protein 3 isoform X1) c4(310613) c5( ) c6( ) c7(iC, T, sS, d, hd, nB, aW, Lf, XY, cj, aT, bnt, ci, as, bu, cm, bz, eA, zn); #c1(SHC3) c2(XP_011517088) c3(SHC-transforming protein 3 isoform X2) c4( ) c5(310614, 310615, 310616, 310617, 310618, 310619, 310620, 310621, 310622) c6( ) c7(iC, T, sS, d, hd, nB, aW, Lf, XY, cj, aT, bnt, ci, as, bu, cm, bz, eA, zn); #c1(SHC4) c2(NP_976224) c3(SHC-transforming protein 4) c4(262993) c5(262994, 262995, 262996, 262997, 262998, 262999) c6( ) c7(Y, cd); #c1(SHC4) c2(XP_011519854) c3(SHC-transforming protein 4 isoform X2) c4(314591) c5(314592, 314593, 314594, 314595, 314596, 314597) c6( ) c7(Y, cd); #c1(SHCBP1) c2(NP_079021) c3(SHC SH2 domain-binding protein 1) c4(216621) c5(216622, 216623, 216624, 216625, 216626, 216627) c6( ) c7(Jm, aGm, mR, aZy, qt, Y, aZw, ym, Fg); #c1(SHF) c2(NP_001288097) c3(SH2 domain-containing adapter protein F isoform a) c4(113531) c5(113532, 113533, 113534, 113535, 113536, 113537) c6( ) c7(cm, ac); #c1(SHF) c2(NP_612365) c3(SH2 domain-containing adapter protein F isoform b) c4(236256, 236257, 236258) c5(236259, 236260, 236261, 236262, 236263, 236264, 236265, 236266, 236267, 236268, 236269, 236270, 236271, 236272, 236273, 236274, 236275, 236276) c6( ) c7(cm, ac); #c1(SHF) c2(XP_011520479) c3(SH2 domain-containing adapter protein F isoform X4) c4( ) c5(315373) c6( ) c7(cm, ac); #c1(SHFM1) c2(NP_006295) c3(26S proteasome complex subunit DSS1) c4(163766) c5(163767, 163768, 163769, 163770, 163771, 163772) c6( ) c7(T, aC, yk, if, aA, bX, qN, eA, ad, arA, aT, hD, ae, aOV, Pw, hM, aW, ff, aF, en, g, t, bN, dB, hk, afd, m, adg); #c1(SHH) c2(NP_000184) c3(sonic hedgehog protein preproprotein) c4(4004) c5(4005, 4006, 4007, 4008, 4009, 4010) c6( ) c7(cDT, uB, T, aC, fd, bB, ci, cDV, Na, bv, aE, cDU, Bc, iv, K, bBp, aJu, eL, aW, az, Az, CT, kt, g, cm, aiv, eM, nB, U, mk, k, BV, iu, asW, RX, ht, n, aj, Nh, jz, bxn, aow, KN, FN, aOM, h, dL, he, Df, Gc, dx, aze, avP, A, MD, iw, Pp, kp, QK, op, oU, kn, ar, iO, bP, asl, dB, hk, bbx, FB, wk, ajd, hR, el, zG, eo, YH, bkE, B, fG, yk, aA, cz, j, Xr, il, V, q, J, eA, eJ, azn, uT, eO, aT, Nc, bK, an, bau, ae, dl, jf, iF, cDX, s, Y, ev, r, en, bZE, LV, f, pb, aUv, sS, cdd, ac, c, nG, agw, gT, bt, Kd, id, aD, ha, jh, zu, Fc, ad, hd, ib, ahk, fb, d, ff, aF, Tp, aDP, atb, ek, cuw, LB, bF, bE, bYB, bkh, auk, adg, cDW, ZW, X, y); #c1(SHISA2) c2(NP_001007539) c3(protein shisa-2 homolog precursor) c4(22788) c5(22789, 22790, 22791, 22792, 22793, 22794) c6( ) c7(af, c); #c1(SHISA3) c2(NP_001073974) c3(protein shisa-3 homolog precursor) c4(36748) c5(36749, 36750, 36751, 36752, 36753, 36754) c6( ) c7(T, bz, eM); #c1(SHISA6) c2(NP_001166932) c3(protein shisa-6 homolog isoform 2 precursor) c4(68643) c5(68644, 68645, 68646, 68647, 68648, 68649) c6( ) c7(Bd, fq, Be); #c1(SHISA6) c2(NP_001166933) c3(protein shisa-6 homolog isoform 3 precursor) c4(68650) c5(68651, 68652, 68653, 68654, 68655, 68656) c6( ) c7(Bd, fq, Be); #c1(SHISA6) c2(NP_997269) c3(protein shisa-6 homolog isoform 1 precursor) c4(265360) c5(265361, 265362, 265363, 265364, 265365, 265366) c6( ) c7(Bd, fq, Be); #c1(SHISA9) c2(NP_001138676) c3(protein shisa-9 isoform 1 precursor) c4(57357, 57358) c5(57359, 57360, 57361, 57362, 57363, 57364, 57365, 57366, 57367, 57368, 57369, 57370) c6( ) c7(bL); #c1(SHISA9) c2(NP_001138677) c3(protein shisa-9 isoform 2 precursor) c4(57371) c5(57372, 57373, 57374, 57375, 57376, 57377, 57378, 57379, 57380, 57381, 57382, 57383) c6( ) c7(bL); #c1(SHISA9) c2(XP_005255596) c3(protein shisa-9 isoform X1) c4(273758) c5( ) c6( ) c7(bL); #c1(SHMT1) c2(NP_001268715) c3(serine hydroxymethyltransferase, cytosolic isoform 3) c4( ) c5(100212, 100213, 100214, 100215, 100216, 100217, 100218, 100219, 100220, 100221, 100222) c6( ) c7(T, fM, fV, iW, e, ha, NT, jh, it, bB, oY, uu, q, aR, ad, md, av, x, Na, NS, fS, M, aT, yg, Nc, dL, he, ae, eL, eu, iL, WW, s, aW, ff, r, en, g, eF, t, iK, bz, bN, ac, eM, lp, bE, wE, bm, dy, k, m, bD, y, bt, Pr, X); #c1(SHMT1) c2(NP_683718) c3(serine hydroxymethyltransferase, cytosolic isoform 2) c4(242440) c5(242441, 242442, 242443, 242444, 242445, 242446) c6( ) c7(T, fM, fV, iW, e, ha, NT, jh, it, bB, oY, uu, q, aR, ad, md, av, x, Na, NS, fS, M, aT, yg, Nc, dL, he, ae, eL, eu, iL, WW, s, aW, ff, r, en, g, eF, t, iK, bz, bN, ac, eM, lp, bE, wE, bm, dy, k, m, bD, y, bt, Pr, X); #c1(SHMT1) c2(XP_005256824) c3(serine hydroxymethyltransferase, cytosolic isoform X1) c4(274530, 274531, 274532, 274533, 274534, 274535) c5(274536, 274537, 274538, 274539, 274540, 274541, 274542, 274543, 274544, 274545, 274546, 274547, 274548) c6( ) c7(T, fM, fV, iW, e, ha, NT, jh, it, bB, oY, uu, q, aR, ad, md, av, x, Na, NS, fS, M, aT, yg, Nc, dL, he, ae, eL, eu, iL, WW, s, aW, ff, r, en, g, eF, t, iK, bz, bN, ac, eM, lp, bE, wE, bm, dy, k, m, bD, y, bt, Pr, X); #c1(SHMT1) c2(XP_011522294) c3(serine hydroxymethyltransferase, cytosolic isoform X2) c4(316954) c5(316955, 316956, 316957, 316958, 316959, 316960, 316961, 316962, 316963, 316964, 316965, 316966, 316967, 316968, 316969, 316970, 316971, 316972) c6( ) c7(T, fM, fV, iW, e, ha, NT, jh, it, bB, oY, uu, q, aR, ad, md, av, x, Na, NS, fS, M, aT, yg, Nc, dL, he, ae, eL, eu, iL, WW, s, aW, ff, r, en, g, eF, t, ik, bz, bN, ac, eM, lp, bE, wE, bm, dy, k, m, bD, y, bt, Pr, X); #c1(SHMT2) c2(NP_001159828) c3(serine hydroxymethyltransferase, mitochondrial isoform 2 precursor) c4(64811) c5(64812, 64813, 64814, 64815, 64816, 64817) c6( ) c7(Na, T, iW, Pr, Nc, bz, it); #c1(SHMT2) c2(NP_001159830) c3(serine hydroxymethyltransferase, mitochondrial isoform 3) c4(64818) c5(64819, 64820, 64821, 64822, 64823, 64824, 64825, 64826, 64827, 64828, 64829) c6( ) c7(Na, T, iW, Pr, Nc, bz, it); #c1(SHMT2) c2(NP_005403) c3(serine hydroxymethyltransferase, mitochondrial isoform 1 precursor) c4(155755) c5(155756, 155757, 155758, 155759, 155760, 155761, 155762, 155763, 155764) c6( ) c7(Na, T, iW, Pr, Nc, bz, it); #c1(SHMT2) c2(XP_011536980) c3(serine hydroxymethyltransferase, mitochondrial isoform X1) c4(334668) c5(334669, 334670, 334671, 334672, 334673, 334674) c6( ) c7(Na, T, iW, Pr, Nc, bz, it); #c1(SHOC2) c2(NP_001255968) c3(leucine-rich repeat protein SHOC-2 isoform 2) c4(94982, 94983) c5(94984, 94985, 94986, 94987, 94988, 94989, 94990, 94991, 94992, 94993, 94994, 94995) c6( ) c7(ck, T, yS, cDZ, eZ, afB, cDY, cxs, By); #c1(SHOC2) c2(NP_031399) c3(leucine-rich repeat protein SHOC-2 isoform 1) c4(172252) c5(172253, 172254, 172255, 172256, 172257, 172258) c6( ) c7(ck, T, yS, cDZ, eZ, afB, cDY, cxs, By); #c1(SHOX) c2(NP_000442) c3(short stature homeobox protein isoform SHOXa) c4(9039) c5(9040, 9041, 9042, 9043, 9044, 9045, 9046, 9047, 9048, 9049) c6( ) c7(bBM, amj, vJ, il, anV, Nn, zf, Vv, UD, rD, K, aDC, cEa, xs, eH, uY, VK, eF, zZ, bQ, dr, bdu, bfP, bpY, lq, aoJ, bxG, xb); #c1(SHOX) c2(NP_006874) c3(short stature homeobox protein isoform SHOXb) c4(168759) c5(168760, 168761, 168762, 168763, 168764, 168765, 168766, 168767, 168768, 168769, 168770) c6( ) c7(bBM, amj, vJ, il, anV, Nn, zf, Vv, UD, rD, K, aDC, cEa, xs, eH, uY, VK, eF, zZ, bQ, dr, bdu, bfP, bpY, lq, aoJ, bxG, xb); #c1(SHOX2) c2(NP_001157150) c3(short stature homeobox protein 2 isoform c) c4(62083, 62084) c5(62085, 62086, 62087, 62088, 62089, 62090, 62091, 62092) c6( ) c7(av, bpY, n, aNL, bK, VK, bz, B, dr, bdu, aa); #c1(SHOX2) c2(NP_003021) c3(short stature homeobox protein 2 isoform b) c4(134522) c5(134523, 134524, 134525, 134526, 134527, 134528, 134529) c6( ) c7(av, bpY, n, aNL, bK, VK, bz, B, dr, bdu, aa); #c1(SHOX2) c2(NP_006875) c3(short stature homeobox protein 2 isoform a) c4(168771) c5(168772, 168773, 168774, 168775, 168776, 168777) c6( ) c7(av, bpY, n, aNL, bK, VK, bz, B, dr, bdu, aa); #c1(SHOX2) c2(XP_006713790) c3(short stature homeobox protein 2 isoform X1) c4( ) c5(287360) c6( ) c7(av, bpY, n, aNL, bK, VK, bz, B, dr, bdu, aa); #c1(SHOX2) c2(XP_006713791) c3(short stature homeobox protein 2 isoform X3) c4( ) c5(287361, 287362, 287363, 287364, 287365, 287366, 287367, 287368, 287369, 287370, 287371) c6( ) c7(av, bpY, n, aNL, bK, VK, bz, B, dr, bdu, aa); #c1(SHPK) c2(NP_037408) c3(sedoheptulokinase) c4(175353) c5(175354, 175355, 175356, 175357, 175358, 175359) c6( ) c7(vr); #c1(SHPRH) c2(NP_001036148) c3(E3 ubiquitin-protein ligase SHPRH isoform a) c4(33990, 33991) c5(33992, 33993, 33994, 33995, 33996, 33997, 33998, 33999, 34000, 34001, 34002, 34003, 34004, 34005, 34006, 34007) c6( ) c7(K); #c1(SHPRH) c2(NP_775105) c3(E3 ubiquitin-protein ligase SHPRH isoform b) c4(249522, 249523) c5(249524, 249525, 249526, 249527, 249528, 249529, 249530, 249531, 249532, 249533, 249534, 249535) c6( ) c7(K); #c1(SHPRH) c2(XP_006715503) c3(E3 ubiquitin-protein ligase SHPRH isoform X2) c4(289602) c5( ) c6( ) c7(K); #c1(SHPRH) c2(XP_011534023) c3(E3 ubiquitin-protein ligase SHPRH isoform X6) c4( ) c5(330195, 330196, 330197, 330198, 330199, 330200) c6( ) c7(K); #c1(SHQ1) c2(NP_060600) c3(protein SHQ1 homolog) c4(196790) c5(196791, 196792, 196793, 196794, 196795, 196796) c6( ) c7(bfG, sb); #c1(SHROOM2) c2(NP_001640) c3(protein Shroom2) c4(119561, 119562, (19563, 119564) c5(119565, 119566, 119567, 119568, 119569, 119570, 119571, 119572, 119573) c6( ) c7(q, r); #c1(SHROOM2) c2(XP_011543808) c3(protein Shroom2 isoform X2) c4( ) c5(342944, 342945, 342946, 342947, 342948, 342949, 342950, 342951, 342952, 342953, 342954, 342955, 342956, 342957, 342958) c6( ) c7(q, r); #c1(SHROOM3) c2(NP_065910) c3 (protein Shroom3) c4(206176) c5(206177, 206178, 206179, 206180, 206181, 206182) c6( ) c7(cc, c, Y, akY, dy, j); #c1(SHROOM3) c2(XP_005263222) c3 (protein Shroom3 isoform X6) c4( ) c5(278359, 278360, 278361, 278362, 278363, 278364, 278365) c6( ) c7(cc, c, Y, akY, dy, j); #c1(SHROOM3) c2(XP_011530462) c3(protein Shroom3 isoform X5) c4(326326, 326327) c5(326328, 326329, 326330, 326331, 326332, 326333) c6( ) c7(cc, c, Y, akY, dy, j); #c1(SHROOM4) c2(NP_065768) c3(protein Shroom4) c4(205289) c5(205290, 205291, 205292, 205293, 205294, 205295, 205296, 205297, 205298, 205299, 205300, 205301, 205302, 205303, 205304, 205305, 205306, 205307, 205308) c6( ) c7(nf, nA, cEb); #c1(SHROOM4) c2(XP_006724653) c3(protein Shroom4 isoform X2) c4(296177) c5( ) c6( ) c7(nf, nA, cEb); #c1(SI) c2(NP_001032) c3(sucrase-isomaltase, intestinal) c4(31510) c5( ) c6( ) c7(Wv, bL, T, bE, ev, ju, zR, cj, cEc, lG, aj, bB, ji, g, bq, q, J, WW); #c1(SIAE) c2(NP_001186851) c3(sialate O-acetylesterase isoform 2) c4( ) c5(78710, 78711, 78712, 78713, 78714) c6( ) c7(cEd, A, bK, y, bc); #c1(SIAE) c2(NP_733746) c3(sialate O-acetylesterase isoform 1 precursor) c4(246918, 246919) c5(246920, 246921, 246922, 246923, 246924, 246925, 246926) c6( ) c7(cEd, A, bK, y, bc); #c1(SIAH1) c2(NP_001006611) c3(E3 ubiquitin-protein ligase SIAH1 isoform b) c4(22199, 22200, 22201) c5(22202, 22203, 22204, 22205, 22206, 22207, 22208, 22209, 22210, 22211, 22212, 22213, 22214, 22215, 22216) c6( ) c7(bF, T, aC, bE, aW, aA, aT, bB, GA, aF, K, an, B); #c1(SIAH1) c2(XP_011521581) c3(E3 ubiquitin-protein ligase SIAH1 isoform X1) c4( ) c5(316311, 316312, 316313, 316314, 316315, 316316, 316317, 316318, 316319, 316320, 316321, 316322, 316323, 316324, 316325, 316326, 316327, 316328, 316329, 316330, 316331, 316332, 316333, 316334, 316335, 316336, 316337, 316338, 316339, 316340, 316341, 316342, 316343, 316344, 316345, 316346, 316347, 316348, 316349, 316350, 316351, 316352, 316353, 316354, 316355, 316356, 316357, 316358, 316359, 316360, 316361, 316362, 316363, 316364, 316365, 316366, 316367, 316368, 316369, 316370, 316371, 316372, 316373, 316374, 316375, 316376, 316377, 316378, 316379, 316380, 316381, 316382, 316383, 316384, 316385, 316386, 316387, 316388, 316389, 316390, 316391, 316392, 316393, 316394, 316395, 316396, 316397, 316398, 316399, 316400, 316401, 316402, 316403, 316404) c6( ) c7(bF, T, aC, bE, aW, aA, aT, bB, GA, aF, K, an, B); #c1(SIAH2) c2(NP_005058) c3(E3 ubiquitin-protein ligase SIAH2) c4(152497) c5(152498, 152499, 152500, 152501, 152502, 152503, 152504) c6( ) c7(ck, ae, T, aW, Y, aw, aT, ZQ, Fb, AO, bz, B, ad); #c1(SIDT1) c2(NP_060169) c3(SID1 transmembrane family member 1 precursor) c4(194606) c5(194607, 194608, 194609, 194610, 194611, 194612) c6( ) c7(asp); #c1(SIGIRR) c2(NP_001128526) c3(single Ig IL-1-related receptor) c4(50166) c5(50167, 50168, 50169, 50170, 50171, 50172, 50173, 50174, 50175, 50176, 50177, 50178, 50179, 50180) c6( ) c7(le, bM, jx, afr, ls, aDE); #c1(SIGIRR) c2(XP_005253101) c3(single Ig IL-1-related receptor isoform X1) c4(272160) c5(272161, 272162, 272163, 272164, 272165, 272166, 272167, 272168, 272169, 272170, 272171) c6( ) c7(le, bM, jx, aFr, ls, aDE); #c1(SIGLEC1) c2(XP_011527630) c3(sialoadhesin isoform X1) c4(322516, 322517) c5(322518, 322519, 322520, 322521, 322522, 322523, 322524, 322525) c6(322526, 322527) c7(bM, T, fR, ds, eZ, h, y, pb); #c1(SIGLEC1) c2(XP_011527631) c3(sialoadhesin isoform X2) c4(322528) c5(322529, 322530, 322531, 322532, 322533, 322534, 322535, 322536) c6(322537, 322538) c7(bM, T, fR, ds, eZ, h, y, pb); #c1(SIGLEC1) c2(XP_011527634) c3(sialoadhesin isoform X5) c4( ) c5(322539, 322540, 322541, 322542, 322543, 322544, 322545, 322546) c6(322547, 322548) c7(bM, T, fR, ds, eZ, h, y, pb); #c1(SIGLEC11) c2(NP_001128635) c3(sialic acid-binding Ig-like lectin ff isoform 2 precursor) c4(50412, 50413, 50414) c5(50415, 50416, 50417, 50418, 50419, 50420, 50421, 50422, 50423, 50424, 50425) c6( ) c7(aPK); #c1(SIGLEC11) c2(NP_443116) c3(sialic acid-binding Ig-like lectin 11 isoform 1 precursor) c4(229506) c5(229507, 229508, 229509, 229510, 229511, 229512) c6( ) c7(aPK); #c1(SIGLEC11) c2(XP_005258533) c3(sialic acid-binding Ig-like lectin 11 isoform X1) c4( ) c5(275691, 275692, 275693, 275694, 275695, 275696) c6( ) c7(aPK); #c1(SIGLEC(1) c2(XP_005258534) c3(sialic acid-binding Ig-like lectin 11 isoform X2) c4( ) c5(275697, 275698, 275699, 275700, 275701) c6( ) c7(aPK); #c1(SIGLEC14) c2(NP_001092082) c3(sialic acid-binding Ig-like lectin 14 precursor) c4(39296) c5(39297, 39298, 39299, 39300, 39301, 39302) c6( ) c7(cEe, jT, at); #c1(SIGLEC5) c2(NP_003821) c3(sialic acid-binding Ig-like lectin 5 precursor) c4(141159) c5(141160, 141161, 141162, 141163, 141164, 141165, 141166, 141167, 141168, 141169) c6( ) c7(jT, bt); #c1(SIGLEC7) c2(NP_001264130) c3(sialic acid-binding Ig-like lectin 7 isoform 3 precursor) c4( ) c5(97537, 97538, 97539, 97540, 97541, 97542) c6( ) c7(fR, bO, aA, bB, aj, OY, hF, eA, aR, amW, no, ec, aCJ, aql, dz, K, hS, zy, H, RG, WZ, jf, eG, LC, Y, Ls, aF, eE, cm, ck, c, iT, Fi, bt, it, L); #c1(SIGLEC7) c2(NP_055200) c3(sialic acid-binding Ig-like lectin 7 isoform 1 precursor) c4(179753) c5(179754, 179755, 179756, 179757, 179758, 179759) c6( ) c7(fR, bO, aA, bB, aj, OY, hF, eA, aR, amW, no, ec, aCJ, aql, dz, K, hS, zy, H, RG, WZ, jf, eG, LC, Y, Ls, aF, eE, cm, ck, c, iT, Fi, bt, it, L); #c1(SIGLEC7) c2(NP_057627) c3(sialic acid-binding Ig-like lectin 7 isoform 2 precursor) c4(191940, 191941) c5(191942, 191943, 191944, 191945, 191946, 191947) c6( ) c7(fR, bO, aA, bB, aj, OY, hF, eA, aR, amW, no, ec, aCJ, aql, dz, K, hS, zy, H, RG, WZ, jf, eG, LC, Y, Ls, aF, eE, cm, ck, c, iT, Fi, bt, it, L); #c1(SIGLEC8) c2(NP_055257) c3(sialic acid-binding Ig-like lectin & precursor) c4(180094) c5(180095, 180096, 180097, 180098, 180099, 180100) c6( ) c7(bM, akc); #c1(SIGLEC8) c2(XP_011525037) c3(sialic acid-binding Ig-like lectin 8 isoform X2) c4(319493, 319494) c5(319495, 319496, 319497, 319498, 319499, 319500, 319501, 319502, 319503, 319504, 319505, 319506) c6( ) c7(bM, akc); #c1(SIGLEC9) c2(NP_001185487) c3(sialic acid-binding Ig-like lectin 9 isoform 1 precursor) c4(75747) c5(75748, 75749, 75750, 75751, 75752, 75753) c6( ) c7(ck, C); #c1(SIGLEC9) c2(NP_055256) c3(sialic acid-binding Ig-like lectin 9 isoform 2 precursor) c4(180087) c5(180088, 180089, 180090, 180091, 180092, 180093) c6( ) c7(ck, C); #c1(SIGLEC9) c2(XP_011525034) c3(sialic acid-binding Ig-like lectin 9 isoform X4) c4(319489) c5(319490, 319491, 319492) c6( ) c7(ck, C); #c1(SIGMAR1) c2(NP_001269136) c3(sigma non-opioid intracellular receptor 1 isoform 8) c4(100536) c5(100537, 100538, 100539, 100540, 100541, 100542, 100543, 100544, 100545, 100546, 100547) c6( ) c7(FW, ot, c, axR, WY, sO, aW, du, Ol, U, aT, bO, jx, cu, lo, h, af, cEf, xt); #c1(SIGMAR1) c2(NP_005857) c3(sigma non-opioid intracellular receptor 1 isoform 1) c4(159974, 159975) c5(159976, 159977, 159978, 159979, 159980, 159981, 159982) c6( ) c7(FW, ot, c, axR, WY, s, aW, dJ, Ol, U, aT, bO, jx, cu, lo, h, af, cEf, xt); #c1(SIGMAR1) c2(NP_671513) c3(sigma non-opioid intracellular receptor 1 isoform 2) c4(242051) c5(242052, 242053, 242054, 242055, 242056, 242057) c6( ) c7(FW, ot, c, axR, WY, sO, aW, dJ, OI, U, aT, bO, jx, cu, lo, h, af, cEf, xt); #c1(SIK1) c2(NP_775490) c3(serine/threonine-protein kinase SIK1) c4(250084) c5(250085, 250086, 250087, 250088, 250089, 250090) c6( ) c7(T, aW, Y, mx, aT, cz, alJ, K, MC, eW); #c1(SIK2) c2(NP_056006) c3(serine/threonine-protein kinase SIK2) c4(185075) c5(185076, 185077, 185078, 185079, 185080, 185081) c6( ) c7(bL, fc, v, j, m, cP, t); #c1(SIK2) c2(XP_011541019) c3(serine/threonine-protein kinase SIK2 isoform X2) c4( ) c5(339558, 339559, 339560) c6( ) c7(bL, fc, v, j, m, cP, t); #c1(SIK3) c2(NP_001268678) c3(serine/threonine-protein kinase SIK3 isoform 3) c4(100126, 100127) c5(100128, 100129, 100130, 100131, 100132, 100133, 100134, 100135, 100136, 100137, 100138, 100139) c6( ) c7(aeP, t, T, m, be); #c1(SIK3) c2(NP_079440) c3(serine/threonine-protein kinase SIK3 isoform 1) c4(218151, 218152) c5(218153, 218154, 218155, 218156, 218157, 218158, 218159, 218160, 218161, 218162, 218163, 218164) c6( ) c7(aeP, t, T, m, be); #c1(SIK3) c2(XP_005271541) c3(serine/threonine-protein kinase SIK3 isoform X4) c4(283448, 283449) c5(283450, 283451, 283452, 283453, 283454, 283455, 283456, 283457, 283458, 283459, 283460, 283461) c6( ) c7(aeP, t, T, m, be); #c1(SIL1) c2(NP_071909) c3(nucleotide exchange factor SIL1 precursor) c4( ) c5(212231, 212232, 212233, 212234, 212235, 212236, 212237, 212238, 212239, 212240, 212241, 212242, 212243, 212244, 212245, 212246, 212247, 212248, 212249, 212250, 212251, 212252, 212253, 212254, 212255, 212256, 212257, 212258) c6( ) c7(Ng, alM, adr, rz, oj, aT, nA, lM, H, bt, ad, bN, L); #c1(SIL1) c2(XP_011541872) c3(nucleotide exchange factor SIL1 isoform X1) c4(340408, 340409, 340410, 340411, 340412) c5(340413, 340414, 340415, 340416, 340417, 340418) c6( ) c7(Ng, alM, adr, rz, oj, aT, nA, lM, H, bt, ad, bN, L); #c1(SIM1) c2(XP_005267157) c3(single-minded homolog 1 isoform X1) c4(280805) c5(280806, 280807, 280808, 280809, 280810, 280811, 280812, 280813, 280814, 280815, 280816) c6( ) c7(aC, VT, aT, dG, cbo, qM, bX, v, rc); #c1(SIM2) c2(NP_005060) c3(single-minded homolog 2 long isoform) c4(152505) c5(152506, 152507, 152508, 152509, 152510, 152511, 152512, 152513) c6( ) c7(G, T, bB, j, ci, nA, pz, eA, ad, Na, anj, aE, aT, ZW, ae, d, cH, aW, Az, bQ, f, EW, GU, dB, bE, kS, X); #c1(SIM2) c2(NP_033664) c3(single-minded homolog 2 short isoform) c4(172296) c5(172297, 172298, 172299, 172300, 172301, (72302) c6( ) c7(G, T, bB, j, ci, nA, pz, eA, ad, Na, anj, aE, aT, ZW, ae, d, cH, aW, Az, bQ, f, EW, GU, dB, bE, kS, X); #c1(SIN3A) c2(XP_006720530) c3(paired amphipathic helix protein Sin3a isoform X1) c4(293542) c5(293543, 293544, 293545, 293546, 293547, 293548, 293549, 293550, 293551, 293552, 293553, 293554, 293555, 293556, 293557) c6( ) c7(bxy, av, T, eM, aXK, aW, aT, vs, bz, iu); #c1(SIN3B) c2(NP_001284524) c3(paired amphipathic helix protein Sin3b isoform 2) c4(111965) c5(111966, 111967, 111968, 111969, 111970, 111971, 111972) c6( ) c7(T, d, aE, aT, kp, K); #c1(SIN3B) c2(NP_056075) c3(paired amphipathic helix protein Sin3b isoform 1) c4(185533) c5(185534, 185535, 185536, 185537, 185538, 185539) c6( ) c7(T, d, aE, aT, kp, K); #c1(SIPA1) c2(NP_694985) c3(signal-induced proliferation-associated protein 1) c4(245562, 245563, 245564) c5(245565, 245566, 245567, 245568, 245569, 245570, 245571, 245572, 245573, 245574, 245575, 245576, 245577, 245578, 245579, 245580, 245581, 245582, 245583, 245584, 245585, 245586, 245587) c6( ) c7(qw, T, aGC, iU, aW, Y, if, oW, aT, ajp, qN, bw, bz, br); #c1(SIPA1L2) c2(NP_065859) c3(signal-induced proliferation-associated 1-like protein 2) c4(205933) c5(205934, 205935, 205936, 205937, 205938, 205939, 205940, 205941, 205942, 205943) c6( ) c7(bM, c); #c1(SIPA1L2) c2(XP_011542547) c3(signal-induced proliferation-associated 1-like protein 2 isoform X2) c4(341089) c5(341090, 341091, 341092, 341093, 341094, 341095) c6( ) c7(bM, c); #c1(SIPA1L3) c2(NP_055888) c3(signal-induced proliferation-associated 1-like protein 3) c4(184203) c5(184204, 184205, 184206, 184207, 184208, 184209) c6( ) c7(cd); #c1(SIPA1L3) c2(XP_011524961) c3(signal-induced proliferation-associated 1-like protein 3 isoform X3) c4( ) c5(319460) c6( ) c7(cd); #c1(SIRPA) c2(NP_542970) c3(tyrosine-protein phosphatase non-receptor type substrate 1 precursor) c4(233121) c5(233122, 233123, 233124, 233125, 233126, 233127, 233128, 233129, 233130, 233131) c6( ) c7(T, yS, aw, cEg, n, bS, ct, Ea, ci, pz, aHj, aT, K, d, ND, xR, aW, S, as, Z, yR, dB, m, afW, bt); #c1(SIRPA) c2(XP_011527474) c3(tyrosine-protein phosphatase non-receptor type substrate 1 isoform X1) c4(322230) c5(322231, 322232, 322233, 322234, 322235, 322236) c6( ) c7(T, yS, aw, cEg, n, bS, ct, Ea, ci, pz, aHj, aT, K, d, ND, xR, aW, S, as, Z, yR, dB, m, afW, bt); #c1SIRPB1) c2(NP_001077379) c3(signal-regulatory protein beta-1 isoform 2 precursor) c4(37802) c5(37803, 37804, 37805, 37806, 37807, 37808) c6( ) c7(iW, zv); #c1(SIRPB1) c2(XP_005260701) c3(signal-regulatory protein beta-1 isoform X7) c4(276817) c5(276818, 276819, 276820, 276821, 276822, 276823) c6( ) c7(iW, zv); #c1(SIRPB1) c2(XP_011527433) c3(signal-regulatory protein beta-1 isoform X1) c4(322157, 322158) c5(322159, 322160, 322161, 322162, 322163, 322164) c6( ) c7(iW, zv); #c1(SIRPB1) c2(XP_011527435) c3(signal-regulatory protein beta-1 isoform X1) c4(322165) c5(322166, 322167, 322168, 322169, 322170, 322171) c6( ) c7(iW, zv); #c1(SIRPB1) c2(XP_011527436) c3 (signal-regulatory protein beta-1 isoform X2) c4(322172) c5(322173, 322174, 322175, 322176, 322177, 322178) c6( ) c7(iW, zv); #c1(SIRPB1) c2(XP_011527437) c3(signal-regulatory protein beta-1 isoform X3) c4( ) c5(322179, 322180, 322181, 322182, 322183, 322184) c6( ) c7(iW, zv); #c1(SIRPG) c2(NP_001034597) c3(signal-regulatory protein gamma isoform 3 precursor) c4(31900) c5(31901, 31902, 31903, 31904, 31905, 31906, 31907, 31908, 31909, 31910) c6( ) c7(vH, A, aw, eZ, ND); #c1(SIRPG) c2(NP_061026) c3(signal-regulatory protein gamma isoform 1 precursor) c4(199035) c5(199036, 199037, 199038, 199039, 199040, 199041, 199042) c6( ) c7(vH, A, aw, eZ, ND); #c1(SIRPG) c2(NP_543006) c3(signal-regulatory protein gamma isoform 2 precursor) c4(233215) c5(233216, 233217, 233218, 233219, 233220, 233221) c6( ) c7(vH, A, aw, eZ, ND); #c1(SIRPG) c2(XP_011527588) c3(signal-regulatory protein gamma isoform X1) c4(322455) c5(322456, 322457, 322458, 322459, 322460) c6( ) c7(vH, A, aw, eZ, ND); #c1(SIRT1) c2(NP_001135970) c3(NAD-dependent protein deacetylase sirtuin-1 isoform b) c4( ) c5(53774, 53775, 53776, 53777, 53778, 53779, 53780, 53781, 53782, 53783, 53784, 53785, 53786, 53787, 53788, 53789, 53790, 53791, 53792, 53793, 53794, 53795, 53796, 53797, 53798, 53799, 53800, 53801, 53802, 53803, 53804, 53805, 53806, 53807, 53808, 53809, 53810, 53811, 53812, 53813, 53814, 53815, 53816, 53817, 53818, 53819, 53820, 53821, 53822, 53823, 53824, 53825, 53826, 53827, 53828, 53829, 53830, 53831, 53832, 53833, 53834, 53835, 53836, 53837, 53838, 53839, 53840, 53841, 53842, 53843, 53844, 53845, 53846, 53847, 53848, 53849, 53850, 53851, 53852, 53853, 53854, 53855, 53856, 53857, 53858, 53859, 53860, 53861, 53862, 53863, 53864, 53865, 53866, 53867, 53868) c6(53869, 53870, 53871) c7(oO, T, cEi, aoq, ea, e, bO, fd, bB, ci, mF, GK, xE, aE, Bc, K, qM, hH, bYL, aa, DA, aW, fc, hl, CT, Ku, g, cm, axp, cEj, eM, nB, U, vy, uJ, Hb, m, aNn, cZ, dD, Ol, aWG, aw, aj, qW, cd, cc, rC, kH, h, jy, eV, bta, dd, A, bux, adF, aq, kp, hq, cA, dB, Qu, ajn, at, E, fL, WL, B, adG, vQ, bL, fM, eX, aA, cR, tc, v, j, C, V, q, J, BA, BJ, MR, adC, aT, cy, dz, bK, bq, ae, af, bA, jf, s, iU, oV, Y, r, en, bSy, t, kC, iK, ck, rR, wg, iM, c, mf, dy, Da, fD, bt, LS, wi, iW, ha, dG, Zh, eq, mC, ad, hX, fU, av, hd, sO, ji, atr, je, lR, qt, ff, cr, eZ, dq, aF, iV, iR, bz, Ek, ek, bE, gZ, apv, cEh, aS, cP, ei, lC, iu, y); #c1(SIRT1) c2(NP_036370) c3(NAD-dependent protein deacetylase sirtuin-1 isoform a) c4(173305, (73306) c5(173307, 173308, 173309, 173310, 173311, 173312, 173313, 173314, 173315) c6( ) c7(oO, T, cEi, aoq, ea, e, bO, fd, bB, ci, mF, GK, xE, aE, Bc, K, qM, hH, bYL, aa, DA, aW, fc, hl, CT, Ku, g, cm, axp, cEj, eM, nB, U, vy, uJ, Hb, m, aNn, cZ, dD, Ol, aWG, aw, aj, qW, cd, cc, rC, kH, h, jy, eV, bta, dd, A, bux, adF, aq, kp, hq, cA, dB, Qu, ajn, at, E, fL, WL, B, adG, vQ, bL, fM, eX, aA, cR, tc, v, j, C, V, q, J, eA, eJ, MR, adC, aT, cy, dz, bK, bq, ae, af, bA, jf, s, iU, oV, Y, r, en, bSy, t, kC, iK, ck, rR, wg, iM, c, mf, dy, Da, fD, bt, LS, wi, iW, ha, dG, Zh, eq, mC, ad, hX, fU, av, hd, sO, ji, atr, je, lR, qt, ff, cr, eZ, dq, aF, iV, iR, bz, Ek, ek, bE, gZ, apv, cEh, aS, cP, ei, lC, iu, y); #c1(SIRT1) c2(XP_011537863) c3(NAD-dependent protein deacetylase sirtuin-1 isoform X2) c4(335374) c5(335375) c6( ) c7(oO, T, cEi, aoq, ea, e, bO, fd, bB, ci, mF, GK, xE, aE, Bc, K, qM, hH, bYL, aa, DA, aW, fc, hl, CT, Ku, g, cm, axp, cEj, eM, nB, U, vy, uJ, Hb, m, aNn, cZ, dD, Ol, aWG, aw, aj, qW, cd, cc, rC, kH, h, jy, eV, bta, dd, A, bux, adF, aq, kp, hq, cA, dB, Qu, ajn, at, E, fL, WL, B, adG, vQ, bL, fM, eX, aA, cR, tc, v, j, C, V, q, J, eA, eJ, MR, adC, aT, cy, dz, bK, bq, ae, af, bA, jf, s, iU, oV, Y, r, en, bSy, t, kC, iK, ck, rR, wg, iM, c, mf, dy, Da, fD, bt, LS, wi, iW, ha, dG, Zh, eq, mC, ad, hX, fU, av, hd, sO, ji, atr, je, lR, qt, ff, cr, eZ, dq, aF, iV, iR, bz, Ek, ek, bE, gZ, apv, cEh, aS, cP, ei, lC, iu, y); #c1(SIRT2) c2(NP_001180215) c3(NAD-dependent protein deacetylase sirtuin-2 isoform 3) c4( ) c5(73442, 73443, 73444, 73445, 73446, 73447, 73448, 73449, 73450, 73451, 73452, 73453, 73454, 73455, 73456, 73457, 73458, 73459, 73460, 73461, 73462, 73463, 73464, 73465) c6( ) c7(oO, bL, T, kh, bM, bzx, wj, yk, eX, NT, aA, GC, n, aj, bS, v, ct, fd, q, J, ad, eA, aE, MR, Do, hW, aT, hD, eV, K, pj, hB, an, ae, bc, uQ, A, mR, aW, cr, r, eZ, aF, hp, app, eS, iV, Z, t, pb, ck, es, FR, YE, c, cA, eM, atM, U, bt, at, cX, m, gT, aed, Pr, B, y); #c1(SIRT2) c2(NP_036369) c3(NAD-dependent protein deacetylase sirtuin-2 isoform 1) c4(173286, 173287, 173288, 173289, 173290, 173291, 173292) c5(173293, 173294, 173295, 173296, 173297, 173298, 173299, 173300, 173301, 173302, 173303, 173304) c6( ) c7(oO, bL, T, kh, bM, bzx, wj, yk, eX, NT, aA, GC, n, aj, bS, v, ct, fd, q, J, ad, eA, aE, MR, Do, hW, aT, hD, eV, K, pj, hB, an, ae, bc, uQ, A, mR, aW, cr, r, eZ, aF, hp, app, eS, iV, Z, t, pb, ck, es, rR, YE, c, cA, eM, atM, U, bt, at, cX, m, gT, aed, Pr, B, y); #c1(SIRT2) c2(XP_011524956) c3(NAD-dependent protein deacetylase sirtuin-2 isoform X1) c4( ) c5(319440, 319441, 319442, 319443, 319444, 319445) c6( ) c7(oO, bL, T, kh, bM, bzx, wj, yk, eX, NT, aA, GC, n, aj, bS, v, ct, fd, q, J, ad, eA, aE, MR, Do, hW, aT, hD, eV, K, pj, hB, an, ae, bc, uQ, A, mR, aW, cr, r, eZ, aF, hp, app, eS, iV, Z, t, pb, ck, es, rR, YE, c, cA, eM, atM, U, bt, at, cX, m, gT, aed, Pr, B, y); #c1(SIRT2) c2(XP_011524958) c3(NAD-dependent protein deacetylase sirtuin-2 isoform X3) c4(319446, 319447) c5(319448, 319449, 319450, 319451, 319452, 319453, 319454, 319455, 319456, 319457, 319458, 319459) c6( ) c7(oO, bL, T, kh, bM, bzx, wj, yk, eX, NT, aA, GC, n, aj, bS, v, ct, fd, q, J, ad, eA, aE, MR, Do, hW, aT, hD, eV, K, pj, hB, an, ae, bc, uQ, A, mR, aW, cr, r, eZ, aF, hp, app, eS, iV, Z, t, pb, ck, es, rR, YE, c, cA, eM, atM, U, bt, at, cX, m, gT, aed, Pr, B, y); #c1(SIRT3) c2(NP_001017524) c3(NAD-dependent protein deacetylase sirtuin-3, mitochondrial isoform b) c4( ) c5(26559, 26560, 26561, 26562, 26563) c6( ) c7(bL, T, aw, n, hy, v, cd, aT, K, af, B, BN, aW, ff, fc, Mz, dq, en, np, dy, cP, ei, fy, ne); #c1(SIRT3) c2(NP_036371) c3(NAD-dependent protein deacetylase sirtuin-3, mitochondrial isoform a) c4(173316, 173317, 173318) c5(173319, 173320, 173321, 173322, 173323, 173324, 173325, 173326, 173327, 173328, 173329, 173330, 173331) c6( ) c7(bL, T, aw, n, hy, v, cd, aT, K, af, B, BN, aW, ff, fc, Mz, dq, en, np, dy, cP, ei, fy, ne); #c1(SIRT4) c2(NP_036372) c3(NAD-dependent protein deacetylase sirtuin-4) c4(173332) c5(173333, 173334, 173335, 173336, 173337, 173338) c6( ) c7(bL, pk, T, fM); #c1(SIRT5) c2(NP_001180196) c3(NAD-dependent protein deacylase sirtuin-5, mitochondrial isoform 3) c4(73342) c5(73343, 73344, 73345, 73346, 73347, 73348) c6( ) c7(he, iH, aR, c); #c1(SIRT5) c2(NP_001229756) c3(NAD-dependent protein deacylase sirtuin-5, mitochondrial isoform 4) c4( ) c5(85148, 85149, 85150, 85151, 85152) c6( ) c7(he, iH, aR, c); #c1(SIRT5) c2(NP_112534) c3(NAD-dependent protein deacylase sirtuin-5, mitochondrial isoform 2) c4(221330) c5(221331, 221332, 221333, 221334, 221335, 221336) c6( ) c7(he, iH, aR, c); #c1(SIRT5) c2(XP_005249025) c3(NAD-dependent protein deacylase sirtuin-5, mitochondrial isoform X1) c4(268864, 268865) c5(268866, 268867, 268868, 268869, 268870, 268871) c6( ) c7(he, iH, aR, c); #c1(SIRT5) c2(XP_011512746) c3(NAD-dependent protein deacylase sirtuin-5, mitochondrial isoform X4) c4( ) c5(303769) c6( ) c7(he, iH, aR, c); #c1(SIRT6) c2(NP_001180214) c3(NAD-dependent protein deacetylase sirtuin-6 isoform 2) c4(73430) c5(73431, 73432, 73433, 73434, 73435, 73436, 73437, 73438, 73439) c6(73440, 73441) c7(ai, T, wr, xE, MR, bE, aW, yk, lH, CM, fc, aT, bB, aq, at, v, vs, adg, y, bz, B); #c1(SIRT6) c2(NP_057623) c3(NAD-dependent protein deacetylase sirtuin-6 isoform 1) c4(191825) c5(191826, 191827, 191828, 191829, 191830, 191831, 191832, 191833, 191834, 191835, 191836, 191837, 191838, 191839, 191840, 191841, 191842, 191843, 191844, 191845, 191846, 191847, 191848, 191849, 191850, 191851, 191852, 191853, 191854, 191855, 191856, 191857, 191858, 191859, 191860, 191861, 191862, 191863, 191864, 191865, 191866, 191867, 191868, 191869, 191870, 191871, 191872, 191873, 191874, 191875, 191876, 191877, 191878, 191879, 191880, 191881, 191882, 191883, 191884, 191885, 191886, 191887, 191888, 191889, 191890, 191891, 191892, 191893, 191894, 191895, 191896, 191897, 191898, 191899, 191900, 191901, 191902, 191903, 191904, 191905, 191906, 191907, 191908, 191909, 191910, 191911, 191912, 191913, 191914, 191915, 191916, 191917, 191918, 191919, 191920, 191921, 191922) c6(191923, 191924, 191925) c7(ai, T, wr, xE, MR, bE, aW, yk, lH, CM, fc, aT, bB, aq, at, v, vs, adg, y, bz, B); #c1(SIRT6) c2(XP_005259632) c3(NAD-dependent protein deacetylase sirtuin-6 isoform X2) c4(276208) c5(276209, 276210, 276211, 276212, 276213, 276214, 276215, 276216) c6(276217, 276218) c7(ai, T, wr, xE, MR, bE, aW, yk, lH, CM, fc, aT, bB, aq, at, v, vs, adg, y, bz, B); #c1(SIRT7) c2(NP_057622) c3(NAD-dependent protein deacetylase sirtuin-7) c4(191811, 191812) c5(191813, 191814, 191815, 191816, 191817, 191818, 191819, 191820, 191821, 191822, 191823, 191824) c6( ) c7(ck, T, iR, aW, fc, jh, al, r, aq, dL, ci, K, q, ei, eL, B); #c1(SIT1) c2(NP_055265) c3(signaling threshold-regulating transmembrane adapter 1 precursor) c4(180147) c5(180148, 180149, 180150, 180151, 180152, 180153) c6( ) c7(h, sO, BF, af, C); #c1(SIVA1) c2(NP_006418) c3(apoptosis regulatory protein Siva isoform 1) c4(164867) c5(164868, 164869, 164870, 164871, 164872, 164873) c6( ) c7(bB, aW, aDX, aT); #c1(SIVA1) c2(NP_068355) c3(apoptosis regulatory protein Siva isoform 2) c4(209325) c5(209326, 209327, 209328, 209329, 209330, 209331) c6( ) c7(bB, aW, aDX, aT); #c1(SIX1) c2(NP_005973) c3(homeobox protein SIX1) c4(160898) c5(160899, 160900, 160901, 160902, 160903, 160904, 160905) c6( ) c7(HQ, T, dA, Dh, sw, if, RX, uO, ajp, ci, qN, q, er, eA, cEk, bv, iu, yQ, aE, bOL, aT, bpf, aMi, cEl, dH, aW, eH, Az, r, g, eF, t, pZ, eM, auZ, np, m, Bh, B); #c1(SIX2) c2(NP_058628) c3(homeobox protein SIX2) c4(192849) c5(192850, 192851, 192852, 192853, 192854, 192855, 192856) c6( ) c7(es, aW, TR, aJL, aT, dy, ci, j, PJ, fr, er); #c1(SIX3) c2(NP_005404) c3(homeobox protein SIX3) c4(155765) c5(155766, 155767, 155768, 155769, 155770, 155771, 155772) c6( ) c7(aNA, kh, kj, bHF, iE, bPt, aJ, ZW, FB, acz, aq, bd, g, bz, iu); #c1(SIX4) c2(NP_059116) c3(homeobox protein SIX4) c4(193120) c5(193121, 193122, 193123, 193124, 193125, 193126, 193127) c6( ) c7(aCQ); #c1(SIX5) c2(NP_787071) c3(homeobox protein SIX5) c4(252061) c5(252062, 252063, 252064, 252065, 252066, 252067, 252068) c6( ) c7(t, ac, yQ, cEm, dR); #c1(SIX6) c2(NP_031400) c3(homeobox protein SIX6) c4(172259) c5(172260, 172261, 172262, 172263, 172264, 172265, 172266) c6( ) c7(cEo, kn, cZ, x, bDf, kj, pZ, dH, cEn, Be, hn, k, dd, dw, agE); #c1(SKA1) c2(NP_001034624) c3(spindle and kinetochore-associated protein 1) c4(31918, 31919) c5(31920, 31921, 31922, 31923, 31924, 31925, 31926, 31927, 31928, 31929, 31930, 31931, 31932, 31933, 31934, 31935, 31936, 31937, 31938) c6( ) c7(bM, AB); #c1(SKA2) c2(NP_001094065) c3(spindle and kinetochore-associated protein 2 isoform 2) c4(40603) c5(40604, 40605, 40606, 40607, 40608, 40609) c6( ) c7(aW, ft, aT); #c1(SKA2) c2(NP_872426) c3(spindle and kinetochore-associated protein 2 isoform 1) c4(256330, 256331) c5(256332, 256333, 256334, 256335, 256336, 256337, 256338, 256339, 256340, 256341, 256342, 256343) c6( ) c7(aW, ft, aT); #c1(SKAP1) c2(NP_003717) c3(src kinase-associated phosphoprotein 1 isoform 1) c4(140383) c5(140384, 140385, 140386, 140387, 140388, 140389) c6( ) c7(t, m, zn); #c1(SKAP2) c2(NP_001290397) c3(src kinase-associated phosphoprotein 2 isoform 2) c4( ) c5(115340, 115341, 115342, 115343, 115344) c6( ) c7(A, iH, dA, dm, akH, bdp, kp, g); #c1(SKAP2) c2(NP_003921) c3(src kinase-associated phosphoprotein 2 isoform 1) c4(142161, 142162, 142163, 142164) c5(142165, 142166, 142167, 142168, 142169, 142170, 142171, 142172, 142173, 142174, 142175, 142176, 142177, 142178, 142179, 142180, 142181, 142182, 142183) c6( ) c7(A, iH, dA, dm, akH, bdp, kp, g); #c1(SKIL) c2(NP_001138569) c3(ski-like protein isoform 2) c4(57004) c5(57005, 57006, 57007, 57008, 57009, 57010, 57011, 57012, 57013, 57014, 57015, 57016) c6( ) c7(T, s, eM, Y, fG, cEp, ff, LZ, fQ, n, ci, lQ, en, Pk, ad); #c1(SKIL) c2(NP_001234937) c3(ski-like protein isoform 1) c4(87860, 87861, 87862) c5(87863, 87864, 87865, 87866, 87867, 87868, 87869, 87870, 87871, 87872, 87873, 87874, 87875, 87876, 87877, 87878) c6( ) c7(T, s, eM, Y, fG, cEp, ff, LZ, fQ, n, ci, lQ, en, Pk, ad); #c1(SKIV2L) c2(NP_008860) c3(helicase SKI2W) c4(169164, 169165, 169166, 169167, 169168, 169169, 169170, 169171) c5(169172, 169173, 169174, 169175, 169176, 169177, 169178, 169179, 169180, 169181, 169182, 169183, 169184, 169185, 169186, 169187, 169188, 169189, 169190, 169191, 169192, 169193, 169194, 169195, 169196, 169197, 169198, 169199, 169200, 169201, 169202, 169203, 169204, 169205, 169206, 169207, 169208, 169209, 169210, 169211, 169212, 169213, 169214, 169215, 169216, 169217, 169218, 169219, 169220, 169221, 169222, 169223, 169224, 169225, 169226, 169227, 169228, 169229, 169230, 169231, 169232, 169233, 169234, 169235, 169236, 169237, 169238, 169239, 169240, 169241, 169242, 169243, 169244, 169245, 169246, 169247, 169248, 169249, 169250, 169251, 169252, 169253, 169254, 169255, 169256, 169257, 169258, 169259, 169260, 169261, 169262, 169263, 169264, 169265, 169266) c6( ) c7(dD, W, nb, Zz, od, ea, lf, eZ, cm, ah, eq, aak, acd, aYD, Um, cEq); #c1(SKIV2L) c2(XP_006715231) c3(helicase SKI2W isoform X1) c4( ) c5(288849, 288850, 288851, 288852, 288853, 288854, 288855, 288856, 288857, 288858, 288859, 288860, 288861, 288862, 288863, 288864, 288865, 288866, 288867) c6( ) c7(dD, W, nb, Zz, od, ea, lf, eZ, cm, ah, eq, aak, acd, aYD, Um, cEq); #c1(SKIV2L2) c2(NP_056175) c3(superkiller viralicidic activity 2-like 2) c4(186137, 186138) c5(186139, 186140, 186141, 186142, 186143, 186144, 186145, 186146, 186147, 186148, 186149, 186150) c6( ) c7(y); #c1(SKOR1) c2(NP_001244953) c3(SKI family transcriptional corepressor 1) c4(92429) c5(92430, 92431, 92432, 92433, 92434, 92435) c6( ) c7(cF, eR, yL, yY, lR, pj, aGK, aY, pb, awm); #c1(SKOR1) c2(XP_011519850) c3(SKI family transcriptional corepressor 1 isoform X1) c4(314577, 314578) c5(314579, 314580, 314581, 314582, 314583, 314584, 314585, 314586, 314587, 314588, 314589, 314590) c6( ) c7(cF, eR, yL, yY, lR, pj, aGK, aY, pb, awm); #c1(SKOR2) c2(NP_001032891) c3(SKI family transcriptional corepressor 2 isoform 2) c4(31483) c5(31484, 31485, 31486, 31487, 31488, 31489, 31490) c6( ) c7(ik, v); #c1(SKOR2) c2(NP_001264992) c3(SKI family transcriptional corepressor 2 isoform 1) c4(97668) c5(97669, 97670, 97671, 97672, 97673, 97674, 97675) c6( ) c7(ik, v); #c1(SKP1) c2(NP_008861) c3(S-phase kinase-associated protein 1 isoform a) c4(169267, 169268, 169269) c5(169270, 169271, 169272, 169273, 169274, 169275, 169276, 169277, 169278, 169279, 169280, 169281, 169282, 169283, 169284, 169285, 169286, 169287, 169288, 169289, 169290, 169291, 169292, 169293) c6( ) c7(Kv, cm, an, h, afs); #c1(SKP1) c2(NP_733779) c3(S-phase kinase-associated protein 1 isoform b) c4(247059, 247060) c5(247061, 247062, 247063, 247064, 247065, 247066, 247067, 247068, 247069, 247070, 247071, 247072, 247073, 247074) c6( ) c7(Kv, cm, an, h, afs); #c1(SKP2) c2(NP_001230049) c3(S-phase kinase-associated protein 2 isoform 3) c4(85846) c5(85847, 85848, 85849, 85850, 85851) c6( ) c7(T, dD, ea, jh, aA, bB, fd, eq, ci, fr, atp, q, ad, hX, dP, av, ft, hw, aE, il, qK, aT, dL, K, zy, pk, ae, eL, cp, jf, d, s, iU, qt, aW, Y, ff, r, nY, aq, kp, aF, yq, en, g, ir, t, zZ, bz, cm, aJg, ck, RV, bP, eM, nB, bE, aJh, iT, hL, hR, dy, m, bt, it, B); #c1(SKP2) c2(NP_005974) c3(S-phase kinase-associated protein 2 isoform 1) c4(160906, 160907) c5(160908, 160909, 160910, 160911, 160912, 160913, 160914, 160915, 160916, 160917, 160918, 160919, 160920, 160921, 160922) c6( ) c7(T, dD, ea, jh, aA, bB, fd, eq, ci, fr, atp, q, ad, hX, dP, av, ft, hw, aE, il, qK, aT, dL, K, zy, pk, ae, eL, cp, jf, d, s, iU, qt, aW, Y, ff, r, nY, aq, kp, aF, yq, en, g, ir, t, zZ, bz, cm, aJg, ck, RV, bP, eM, nB, bE, aJh, iT, hL, hR, dy, m, bt, it, B); #c1(SKP2) c2(NP_116026) c3(S-phase kinase-associated protein 2 isoform 2) c4(225251) c5(225252, 225253, 225254, 225255, 225256, 225257, 225258) c6( ) c7(T, dD, ea, jh, aA, bB, fd, eq, ci, fr, atp, q, ad, hX, dP, av, ft, hw, aE, il, qK, aT, dL, K, zy, pk, ae, eL, cp, jf, d, s, iU, qt, aW, Y, ff, r, nY, aq, kp, aF, yq, en, g, ir, t, zZ, bz, cm, aJg, ck, RV, bP, eM, nB, bE, aJh, iT, hL, hR, dy, m, bt, it, B); #c1(SLA) c2(NP_001039021) c3(src-like-adapter isoform a) c4(34182, 34183) c5(34184, 34185, 34186, 34187, 34188, 34189, 34190, 34191, 34192, 34193) c6( ) c7(x, qp, qj, k); #c1(SLA) c2(NP_001039022) c3(src-like-adapter isoform b) c4(34194, 34195, 34196, 34197, 34198) c5(34199, 34200, 34201, 34202, 34203, 34204, 34205, 34206) c6( ) c7(x, qp, qj, k); #c1(SLA) c2(NP_001269893) c3(src-like-adapter isoform d) c4( ) c5(103089, 103090, 103091, 103092, 103093, 103094, 103095, 103096, 103097, 103098, 103099, 103100, 103101, 103102, 103103, 103104, 103105, 103106, 103107) c6( ) c7(x, qp, qj, k); #c1(SLA) c2(NP_006739) c3(src-like-adapter isoform c) c4(167614) c5(167615, 167616, 167617, 167618, 167619, 167620, 167621, 167622, 167623, 167624, 167625) c6( ) c7(x, qp, qj, k); #c1(SLA2) c2(NP_115590) c3(src-like-adapter 2 isoform a) c4(223304) c5(223305, 223306, 223307, 223308, 223309, 223310) c6( ) c7(aS); #c1(SLA2) c2(NP_778252) c3(src-like-adapter 2 isoform b) c4(251516) c5(251517, 251518, 251519, 251520, 251521, 251522) c6( ) c7(aS); #c1(SLAIN2) c2(NP_065897) c3(SLAIN motif-containing protein 2) c4(206(29) c5(206130, 206131, 206132, 206133, 206134, 206135) c6( ) c7(h); #c1(SLAIN2) c2(XP_005248178) c3(SLAIN motif-containing protein 2 isoform X1) c4( ) c5(268271) c6( ) c7(h); #c1(SLAMF1) c2(NP_003028) c3(signaling lymphocytic activation molecule precursor) c4(134538, 134539, 134540) c5(134541, 134542, 134543, 134544, 134545, 134546, 134547, 134548, 134549, 134550, 134551, 134552, 134553, 134554, 134555, 134556, 134557, 134558) c6( ) c7(bL, fR, axd, jh, fd, mC, aR, aGW, AW, tT, h, dL, afu, eZ, lo, CQ, kk, hc, cj, at, aEG, fL, y, Ze, abh); #c1(SLAMF1) c2(XP_005245513) c3(signaling lymphocytic activation molecule isoform X1) c4(266676, 266677) c5(266678, 266679, 266680, 266681, 266682, 266683) c6( ) c7(bL, fR, axd, jh, fd, mC, aR, aGW, AW, tT, h, dL, afu, eZ, lo, CQ, kk, hc, cj, at, aEG, fL, y, Ze, abh); #c1(SLAMF1) c2(XP_005245514) c3(signaling lymphocytic activation molecule isoform X2) c4( ) c5(266684, 266685, 266686, 266687, 266688, 266689) c6( ) c7(bL, fR, axd, jh, fd, mC, aR, aGW, AW, tT, h, dL, afu, eZ, lo, CQ, kk, hc, cj, at, aEG, fL, y, Ze, abh); #c1(SLAMF6) c2(NP_001171643) c3(SLAM family member 6 isoform 1 precursor) c4(71093) c5(71094, 71095, 71096, 71097, 71098, 71099) c6( ) c7(akJ, eZ, fd, hD, h, fL, mC); #c1(SLAMF6) c2(NP_001171645) c3(SLAM family member 6 isoform 4 precursor) c4(71100) c5(71101, 71102, 71103, 71104, 71105, 71106) c6( ) c7(akJ, eZ, fd, hD, h, fL, mC); #c1(SLAMF6) c2(NP_443163) c3(SLAM family member 6 isoform 2 precursor) c4(229684) c5(229685, 229686, 229687, 229688, 229689, 229690) c6( ) c7(akJ, eZ, fd, hD, h, fL, mC); #c1(SLAMF7) c2(NP_001269517) c3(SLAM family member 7 isoform b precursor) c4(101763) c5(101764, 101765, 101766, 101767, 101768, 101769) c6( ) c7(ba, aSP, bg, eZ, fd, uz, Tz); #c1(SLAMF7) c2(NP_001269518) c3(SLAM family member 7 isoform c precursor) c4(101770) c5(101771, 101772, 101773, 101774, 101775, 101776) c6( ) c7(ba, aSP, bg, eZ, fd, uz, Tz); #c1(SLAMF7) c2(NP_001269519) c3(SLAM family member 7 isoform d) c4(101777) c5(101778, 101779, 101780, 101781, 101782, 101783) c6( ) c7(ba, aSP, bg, eZ, fd, uz, Tz); #c1(SLAMF7) c2(NP_001269520) c3(SLAM family member 7 isoform e) c4(101784) c5(101785, 101786, 101787, 101788, 101789, 101790) c6( ) c7(ba, aSP, bg, eZ, fd, uz, Tz); #c1(SLAMF7) c2(NP_001269521) c3(SLAM family member 7 isoform f precursor) c4(101791) c5(101792, 101793, 101794, 101795, 101796, 101797) c6( ) c7(ba, aSP, bg, eZ, fd, uz, Tz); #c1(SLAMF7) c2(NP_001269522) c3(SLAM family member 7 isoform g) c4(101798) c5(101799, 101800, 101801, 101802, 101803, 101804) c6( ) c7(ba, aSP, bg, eZ, fd, uz, Tz); #c1(SLAMF7) c2(NP_067004) c3(SLAM family member 7 isoform a precursor) c4(208256) c5(208257, 208258, 208259, 208260, 208261, 208262) c6( ) c7(ba, aSP, bg, eZ, fd, uz, Tz); #c1(SLAMF8) c2(NP_064510) c3(SLAM family member 8 precursor) c4(202705) c5(202706, 202707, 202708, 202709, 202710, 202711, 202712, 202713, 202714, 202715, 202716) c6( ) c7(ey, (x); #c1(SLAMF8) c2(XP_011508081) c3(SLAM family member 8 isoform X6) c4(298574) c5( ) c6( ) c7(By, lx); #c1(SLAMF8) c2(XP_011508082) c3(SLAM family member 8 isoform X7) c4( ) c5(298575) c6( ) c7(ey, lx); #c1(SLBP) c2(NP_006518) c3(histone RNA hairpin-binding protein) c4(165947) c5(165948, 165949, 165950, 165951, 165952, 165953) c6( ) c7(T, di, dc, dJ, ey, ad); #c1(SLC10A1) c2(NP_003040) c3(sodium/bile acid cotransporter) c4(134627) c5(134628, 134629, 134630, 134631, 134632, 134633) c6( ) c7(F, vj, hJ, al, bbT, fQ, aq, lo, wC, dQ, B, EB, bc, hX); #c1(SLC10A2) c2(NP_000443) c3(ileal sodium/bile acid cotransporter) c4(9050) c5(9051, 9052, 9053, 9054, 9055, 9056) c6( ) c7(aC, Ht, pf, oe, qd, al, bB, Wb, aj, bX, jw, cd, q, aR, ad, l, u, pN, ju, RE, wC, cH, r, hJ, fq, cEr, bE, wz, lB); #c1(SLC10A6) c2(NP_932069) c3(solute carrier family 10 member 6) c4(258786) c5(258787, 258788, 258789, 258790, 258791, 258792) c6( ) c7(ND); #c1(SLC10A7) c2(NP_001025169) c3(sodium/bile acid cotransporter 7 isoform b) c4(29383) c5(29384, 29385, 29386, 29387, 29388, 29389, 29390) c6( ) c7(UF); #c1(SLC10A7) c2(NP_001287771) c3(sodium/bile acid cotransporter 7 isoform d) c4(112837) c5(112838, 112839, 112840, 112841, 112842, 112843, 112844, 112845, 112846, 112847) c6( ) c7(UF); #c1(SLC10A7) c2(NP_115504) c3(sodium/bile acid cotransporter 7 isoform c) c4(222962) c5(222963, 222964, 222965, 222966, 222967, 222968) c6( ) c7(UF); #c1(SLC11A1) c2(NP_000569) c3(natural resistance-associated macrophage protein 1) c4(12782, 12783) c5(12784, 12785, 12786, 12787, 12788, 12789, 12790, 12791, 12792, 12793, 12794) c6( ) c7(bL, bM, T, ajy, dD, lk, fR, cEs, bcb, if, ht, al, jx, aA, CH, n, cL, aj, eq, qN, V, zo, o, J, LH, ai, aMB, AW, l, F, bOl, yw, u, ady, cEt, bW, Vr, oW, aT, dV, h, azm, bOp, dL, hH, Xl, eL, aa, uQ, aEV, A, aMm, aW, vj, asD, ge, fe, lo, aF, pD, LJ, aCK, eE, iV, acD, ik, pb, adz, agS, cEu, gk, acC, aQd, ig, aDs, WP, anY, Ny, bRS, at, E, lf, y); #c1(SLC11A1) c2(XP_0067 (2773) c3(natural resistance-associated macrophage protein 1 isoform X3) c4(286832) c5(286833, 286834, 286835, 286836, 286837, 286838, 286839) c6( ) c7(bL, bM, T, ajy, dD, lk, fR, cEs, bcb, if, ht, al, jx, aA, CH, n, cL, aj, eq, qN, V, zo, o, J, LH, ai, aMB, AW, l, F, bOl, yw, u, ady, cEt, bW, Vr, oW, aT, dV, h, azm, bOp, dL, hH, Xl, eL, aa, uQ, aEV, A, aMm, aW, vj, asD, ge, fe, lo, aF, pD, LJ, aCK, eE, iV, acD, ik, pb, adz, agS, cEu, gk, acC, and, ig, aDs, WP, anY, Ny, bRS, at, E, lf, y); #c1(SLC11A2) c2(NP_000608) c3(natural resistance-associated macrophage protein 2 isoform 3) c4( ) c5(13710, 13711, 13712, 13713, 13714, 13715, 13716, 13717, 13718, 13719, 13720, 13721, 13722, 13723) c6( ) c7(cEw, yY, al, cEv, bGZ, ci, Rc, yw, oW, yu, cy, eu, yy, aq, hp, iV, gk, BU, ac, aNv, wO, dy, fD, aNt); #c1(SLC11A2) c2(NP_001167596) c3(natural resistance-associated macrophage protein 2 isoform 1) c4(69380, 69381, 69382, 69383) c5(69384, 69385, 69386, 69387, 69388, 69389, 69390, 69391, 69392, 69393, 69394, 69395, 69396) c6( ) c7(cEw, yY, al, cEv, bGZ, ci, Rc, yw, oW, yu, cy, eu, yy, aq, hp, iV, gk, BU, ac, aNv, wO, dy, fD, aNt); #c1(SLC11A2) c2(NP_001167601) c3(natural resistance-associated macrophage protein 2 isoform 4) c4(69397) c5(69398, 69399, 69400, 69401, 69402, 69403, 69404, 69405, 69406, 69407, 69408) c6( ) c7(cEw, yY, al, cEv, bGZ, ci, Rc, yw, oW, yu, cy, eu, yy, aq, hp, iV, gk, BU, ac, aNv, wO, dy, fD, aNt); #c1(SLC11A2) c2(XP_005268968) c3(natural resistance-associated macrophage protein 2 isoform X2) c4(282253) c5(282254, 282255, 282256) c6( ) c7(cEw, yY, al, cEv, bGZ, ci, Rc, yw, oW, yu, cy, eu, yy, aq, hp, iV, gk, BU, ac, aNv, wO, dy, fD, aNt); #c1(SLC11A2) c2(XP_011536707) c3(natural resistance-associated macrophage protein 2 isoform X1) c4( ) c5(334260, 334261, 334262, 334263, 334264, 334265, 334266, 334267, 334268, 334269, 334270) c6( ) c7(cEw, yY, al, cEv, bGZ, ci, Rc, yw, oW, yu, cy, eu, yy, aq, hp, iV, gk, BU, ac, aNv, wO, dy, fD, aNt); #c1(SLC12A1) c2(NP_000329) c3(solute carrier family 12 member 1 isoform A) c4(7157, 7158, 7159, 7160) c5(7161, 7162, 7163, 7164, 7165, 7166, 7167, 7168, 7169, 7170, 7171, 7172, 7173, 7174, 7175, 7176, 7177, 7178, 7179, 7180, 7181, 7182, 7183, 7184, 7185, 7186, 7187, 7188) c6( ) c7(Tl, s, XD, vj, cEx, Fe, cz, vw, dy, aPz, qa); #c1(SLC12A1) c2(NP_001171761) c3(solute carrier family 12 member 1 isoform F) c4(71526) c5(71527, 71528, 71529, 71530, 71531, 71532, 71533, 71534, 71535, 71536, 71537, 71538, 71539, 71540, 71541, 71542, 71543, 71544, 71545, 71546, 71547, 71548, 71549, 71550, 71551, 71552, 71553, 71554, 71555, 71556, 71557, 71558, 71559, 71560, 71561, 71562, 71563, 71564, 71565, 71566, 71567, 71568, 71569, 71570, 71571, 71572, 71573, 71574, 71575, 71576, 71577, 71578, 71579, 71580, 71581, 71582, 71583, 71584, 71585, 71586, 71587, 71588, 71589, 71590, 71591, 71592, 71593, 71594, 71595, 71596, 71597, 71598, 71599, 71600, 71601, 71602, 71603, 71604, 71605, 71606, 71607, 71608, 71609, 71610, 71611, 71612, 71613, 71614, 71615, 71616, 71617, 71618) c6( ) c7(Tl, s, XD, vj, cEx, Fe, cz, vw, dy, aPz, qa); #c1(SLC12A1) c2(XP_005254662) c3(solute carrier family 12 member 1 isoform X1) c4(273294) c5( ) c6( ) c7(Tl, s, XD, vj, cEx, Fe, cz, vw, dy, aPz, qa); #c1(SLC12A1) c2(XP_005254663) c3(solute carrier family 12 member 1 isoform X2) c4( ) c5(273295) c6( ) c7(Tl, s, XD, vj, cEx, Fe, cz, vw, dy, aPz, qa); #c1(SLC12A1) c2(XP_006720719) c3(solute carrier family 12 member 1 isoform X3) c4( ) c5(293617, 293618, 293619, 293620, 293621) c6( ) c7(Tl, s, XD, vj, cEx, Fe, cz, vw, dy, aPz, qa); #c1(SLC12A2) c2(NP_001243390) c3(solute carrier family 12 member 2 isoform 2) c4(90657) c5(90658, 90659, 90660, 90661, 90662, 90663) c6( ) c7(bM, e, ha, aw, qy, cz, ci, il, zP, eA, ad, cy, K, zQ, d, oV, Fe, aq, xw, ac, dB, mG, mf, B); #c1(SLC12A3) c2(NP_000330) c3(solute carrier family 12 member 3 isoform 1) c4(7189) c5(7190, 7191, 7192, 7193, 7194, 7195) c6( ) c7(bL, aJV, T, dD, hl, eR, ea, al, cz, yO, ci, eq, aY, hF, iu, Mo, ft, Tl, XD, aT, afV, dL, K, aDZ, Xh, Fg, ahE, eL, bA, cgO, jf, cEy, cla, d, aW, Y, ff, Xg, dq, Xj, si, bWB, aPz, ahL, aUO, en, bz, dr, qa, amF, es, eM, iA, dB, cqP, kQ, vy, dy, k, aS, ik, aAE); #c1(SLC12A3) c2(NP_001119579) c3(solute carrier family 12 member 3 isoform 2) c4(44161) c5(44162, 44163, 44164, 44165, 44166, 44167, 44168, 44169, 44170, 44171) c6( ) c7(bL, aJV, T, dD, hl, eR, ea, al, cz, yO, ci, eq, aY, hF, iu, Mo, ft, Tl, XD, aT, afV, dL, K, aDZ, Xh, Fg, ahE, eL, bA, cgO, jf, cEy, cla, d, aW, Y, ff, Xg, dq, Xj, si, bWB, aPz, ahL, aUO, en, bz, dr, qa, amF, es, eM, iA, dB, cqP, kQ, vy, dy, k, aS, ik, aAE); #c1(SLC12A3) c2(NP_001119580) c3(solute carrier family 12 member 3 isoform 3) c4(44172) c5(44173, 44174, 44175, 44176, 44177, 44178) c6( ) c7(bL, aJV, T, dD, hl, eR, ea, al, cz, yO, ci, eq, aY, hF, iu, Mo, ft, Tl, XD, aT, afV, dL, K, aDZ, Xh, Fg, ahE, eL, bA, cgO, jf, cEy, cla, d, aW, Y, ff, Xg, dq, Xj, si, bWB, aPz, ahL, aUO, en, bz, dr, qa, amF, es, eM, iA, dB, cqP, kQ, vy, dy, k, aS, ik, aAE); #c1(SLC12A4) c2(NP_001139433) c3(solute carrier family 12 member 4 isoform b) c4( ) c5(59331, 59332, 59333, 59334, 59335, 59336) c6( ) c7(qb, ta, if, qN); #c1(SLC12A4) c2(NP_001139434) c3(solute carrier family 12 member 4 isoform c) c4(59337) c5(59338, 59339, 59340, 59341, 59342, 59343) c6( ) c7(qb, ta, if, qN); #c1(SLC12A4) c2(NP_001139435) c3(solute carrier family 12 member 4 isoform d) c4(59344) c5(59345, 59346, 59347, 59348, 59349, 59350) c6( ) c7(qb, ta, if, qN); #c1(SLC12A4) c2(NP_001139436) c3(solute carrier family 12 member 4 isoform e) c4(59351) c5(59352, 59353, 59354, 59355, 59356, 59357) c6( ) c7(qb, ta, if, qN); #c1(SLC12A4) c2(NP_005063) c3(solute carrier family 12 member 4 isoform a) c4(152530, 152531) c5(152532, 152533, 152534, 152535, 152536, 152537) c6( ) c7(qb, ta, if, qN); #c1(SLC12A5) c2(NP_001128243) c3(solute carrier family 12 member 5 isoform 1) c4(49746) c5(49747, 49748, 49749, 49750, 49751, 49752) c6( ) c7(bL, Cq, ql, cA, dB, bE, ha, bB, cy, il, cB, akS); #c1(SLC12A5) c2(NP_065759) c3(solute carrier family 12 member 5 isoform 2) c4(205235, 205236, 205237) c5(205238, 205239, 205240, 205241, 205242, 205243, 205244, 205245, 205246, 205247, 205248, 205249, 205250, 205251, 205252, 205253, 205254, 205255) c6( ) c7(bL, Cq, ql, cA, dB, bE, ha, bB, cy, il, cB, akS); #c1(SLC12A6) c2(NP_001035960) c3(solute carrier family 12 member 6 isoform c) c4(33545) c5( ) c6( ) c7(ql, aW, if, ha, qB, U, aT, ta, iD, bK, fu, bX, qN, cjg); #c1(SLC12A6) c2(NP_001035961) c3(solute carrier family (2 member 6 isoform d) c4(33546, 33547, 33548) c5( ) c6( ) c7(ql, aW, if, ha, qB, U, aT, ta, iD, bK, fu, bX, qN, cjg); #c1(SLC12A6) c2(NP_001035962) c3(solute carrier family 12 member 6 isoform e) c4(33549) c5(33550, 33551, 33552, 33553, 33554, 33555) c6( ) c7(ql, aW, if, ha, qB, U, aT, ta, iD, bK, fu, bX, qN, cjg); #c1(SLC12A6) c2(NP_005126) c3(solute carrier family 12 member 6 isoform b) c4(153078) c5(153079, 153080, 153081, 153082, 153083, 153084) c6( ) c7(ql, aW, if, ha, qB, U, aT, ta, iD, bK, fu, bX, qN, cjg); #c1(SLC12A6) c2(XP_006720856) c3(solute carrier family 12 member 6 isoform X2) c4(293746) c5(293747, 293748, 293749, 293750, 293751, 293752, 293753, 293754, 293755, 293756) c6( ) c7(ql, aW, if, ha, qB, U, aT, ta, iD, bK, fu, bX, qN, cjg); #c1(SLC12A6) c2(XP_011520570) c3(solute carrier family 12 member 6 isoform X1) c4(315499) c5(315500, 315501, 315502, 315503, 315504, 315505, 315506, 315507, 315508, 315509, 315510, 315511, 315512, 315513, 315514, 315515, 315516, 315517, 315518, 315519, 315520, 315521, 315522, 315523, 315524, 315525, 315526, 315527, 315528, 315529) c6( ) c7(ql, aW, if, ha, qB, U, aT, ta, iD, bK, fu, bX, qN, cjg); #c1(SLC12A6) c2(XP_011520571) c3(solute carrier family 12 member 6 isoform X3) c4( ) c5(315530, 315531, 315532, 315533, 315534, 315535, 315536, 315537, 315538, 315539, 315540, 315541) c6( ) c7(ql, aW, if, ha, qB, U, aT, ta, iD, bK, fu, bX, qN, cjg); #c1(SLC12A7) c2(NP_006589) c3(solute carrier family 12 member 7) c4(166475, 166476) c5(166477, 166478, 166479, 166480, 166481, 166482, 166483, 166484, 166485, 166486, 166487, 166488) c6( ) c7(aW, aT, ajT, KN); #c1(SLC12A8) c2(NP_001182412) c3(solute carrier family 12 member 8) c4(74883, 74884, 74885, 74886) c5(74887, 74888, 74889, 74890, 74891, 74892, 74893, 74894, 74895, 74896, 74897, 74898, 74899, 74900, 74901, 74902, 74903, 74904, 74905, 74906, 74907, 74908, 74909, 74910, 74911, 74912, 74913, 74914) c6( ) c7(cr, Jr); #c1(SLC12A9) c2(NP_001254741) c3(solute carrier family 12 member 9 isoform 2) c4(94920, 94921) c5(94922, 94923, 94924, 94925, 94926, 94927, 94928, 94929, 94930, 94931, 94932, 94933, 94934, 94935, 94936, 94937, 94938, 94939) c6( ) c7(gh, T, os, pf, if, al, fd, bB, ci, x, bv, aE, aEX, K, hu, eL, bc, Qe, aa, yo, aW, wF, g, as, eF, cm, aAB, eM, nB, AM, iA, iT, wE, cj, k, m, iu, Or, ht, NT, n, alZ, fr, qN, ai, eW, co, zw, kH, h, dL, aJb, pl, ks, aq, kp, Hu, YF, dw, Ld, Ye, bP, dB, pV, fL, WL, it, B, jx, aA, sM, v, j, atp, q, eA, eJ, akU, Vr, aT, cy, jj, yb, ae, ls, s, NE, Y, ev, Lf, r, en, bu, cl, t, f, ik, ck, QG, iM, lI, agf, np, im, rA, bt, hS, Mb, kh, id, jh, oZ, dG, pz, iJ, mC, ad, hX, av, hd, Rq, oW, Dl, d, je, qt, ff, eH, cr, eZ, gE, Vl, aF, iV, iR, KM, bz, LB, gk, bE, bC, y, kS, X); #c1(SLC12A9) c2(NP_001254743) c3(solute carrier family 12 member 9 isoform 3) c4(94940) c5(94941, 94942, 94943, 94944, 94945, 94946, 94947, 94948, 94949, 94950) c6( ) c7(gh, T, os, pf, if, al, fd, bB, ci, x, bv, aE, aEX, K, hu, eL, bc, Qe, aa, yo, aW, wF, g, as, eF, cm, aAB, eM, nB, AM, iA, iT, wE, cj, k, m, iu, Or, ht, NT, n, alZ, fr, qN, ai, eW, co, zw, kH, h, dL, aJb, pl, ks, aq, kp, Hu, YF, dw, Ld, Ye, bP, dB, pV, fL, WL, it, B, jx, aA, sM, v, j, atp, q, eA, eJ, akU, Vr, aT, cy, jj, yb, ae, ls, s, NE, Y, ev, Lf, r, en, bu, cl, t, f, ik, ck, QG, iM, lI, agf, np, im, rA, bt, hS, Mb, kh, id, jh, oZ, dG, pz, iJ, mC, ad, hX, av, hd, Rq, oW, Dl, d, je, qt, ff, eH, cr, eZ, gE, Vl, aF, iV, iR, KM, bz, LB, gk, bE, bC, y, kS, X); #c1(SLC12A9) c2(NP_064631) c3(solute carrier family 12 member 9 isoform 1) c4(203398) c5(203399, 203400, 203401, 203402, 203403, 203404) c6( ) c7(gh, T, os, pf, if, al, fd, bB, ci, x, bv, aE, aEX, K, hu, eL, bc, Qe, aa, yo, aW, wF, g, as, eF, cm, aAB, eM, nB, AM, iA, iT, wE, cj, k, m, iu, Or, ht, NT, n, alZ, fr, qN, ai, eW, co, zw, kH, h, dL, aJb, pl, ks, aq, kp, Hu, YF, dw, Ld, Ye, bP, dB, pV, fL, WL, it, B, jx, aA, sM, v, j, atp, q, eA, eJ, akU, Vr, aT, cy, jj, yb, ae, ls, s, NE, Y, ev, Lf, r, en, bu, cl, t, f, ik, ck, QG, iM, lI, agf, np, im, rA, bt, hS, Mb, kh, id, jh, oZ, dG, pz, iJ, mC, ad, hX, av, hd, Rq, oW, Dl, d, je, qt, ff, eH, cr, eZ, gE, Vl, aF, iV, iR, KM, bz, LB, gk, bE, bC, y, kS, X); #c1(SLC12A9) c2(XP_005250561) c3(solute carrier family 12 member 9 isoform X5) c4( ) c5(270373, 270374, 270375, 270376, 270377, 270378) c6( ) c7(gh, T, os, pf, if, al, fd, bB, ci, x, bv, aE, aEX, K, hu, eL, bc, Qe, aa, yo, aW, wF, g, as, eF, cm, aAB, eM, nB, AM, iA, iT, wE, cj, k, m, iu, Or, ht, NT, n, alZ, fr, qN, ai, eW, co, zw, kH, h, dL, aJb, pl, ks, aq, kp, Hu, YF, dw, Ld, Ye, bP, dB, pV, fL, WL, it, B, jx, aA, sM, v, j, atp, q, eA, eJ, akU, Vr, aT, cy, jj, yb, ae, ls, s, NE, Y, ev, Lf, r, en, bu, cl, t, f, ik, ck, QG, iM, lI, agf, np, im, rA, bt, hS, Mb, kh, id, jh, oZ, dG, pz, iJ, mC, ad, hX, av, hd, Rq, oW, Dl, d, je, qt, ff, eH, cr, eZ, gE, Vl, aF, iV, iR, KM, bz, LB, gk, bE, bC, y, kS, X); #c1(SLC12A9) c2(XP_006716118) c3(solute carrier family 12 member 9 isoform X2) c4(290086, 290087) c5( ) c6( ) c7(gh, T, os, pf, if, al, fd, bB, ci, x, bv, aE, aEX, K, hu, eL, bc, Qe, aa, yo, aW, wF, g, as, eF, cm, aAB, eM, nB, AM, iA, iT, wE, cj, k, m, iu, Or, ht, NT, n, alZ, fr, qN, ai, eW, co, zw, kH, h, dL, aJb, pl, ks, aq, kp, Hu, YF, dw, Ld, Ye, bP, dB, pV, fL, WL, it, B, jx, aA, sM, v, j, atp, q, eA, eJ, akU, Vr, aT, cy, jj, yb, ae, ls, s, NE, Y, ev, Lf, r, en, bu, cl, t, f, ik, ck, QG, iM, lI, agf, np, im, rA, bt, hS, Mb, kh, id, jh, oZ, dG, pz, iJ, mC, ad, hX, av, hd, Rq, oW, Dl, d, je, qt, ff, eH, cr, eZ, gE, Vl, aF, iV, iR, KM, bz, LB, gk, bE, bC, y, kS, X); #c1(SLC13A1) c2(NP_071889) c3(solute carrier family 13 member 1) c4(212113) c5(212114, 212115, 212116, 212117, 212118, 212119) c6( ) c7(bL, aw, dq, k, ade, rv, bN); #c1(SLC13A1) c2(XP_011514817) c3(solute carrier family 13 member 1 isoform X1) c4( ) c5(307483) c6( ) c7(bL, aw, dq, k, ade, rv, bN); #c1(SLC13A1) c2(XP_011514821) c3(solute carrier family 13 member 1 isoform X5) c4(307484) c5(307485, 307486, 307487, 307488, 307489) c6( ) c7(bL, aw, dq, k, ade, rv, bN); #c1(SLC13A2) c2(NP_001139447) c3(solute carrier family 13 member 2 isoform a) c4(59372) c5(59373, 59374, 59375, 59376, 59377, 59378) c6( ) c7(FE, aW, m, aT); #c1(SLC13A2) c2(NP_003975) c3(solute carrier family 13 member 2 isoform b) c4(142580, 142581, 142582) c5(142583, 142584, 142585, 142586, 142587, 142588, 142589, 142590, 142591, 142592, 142593, 142594, 142595, 142596, 142597, 142598, 142599, 142600, 142601) c6( ) c7(FE, aW, m, aT); #c1(SLC13A2) c2(XP_006722228) c3(solute carrier family 13 member 2 isoform X5) c4(294649) c5(294650, 294651, 294652, 294653, 294654) c6( ) c7(FE, aW, m, aT); #c1(SLC13A3) c2(NP_001011554) c3(solute carrier family 13 member 3 isoform b) c4(24648) c5(24649, 24650, 24651, 24652, 24653) c6( ) c7(bL); #c1(SLC13A3) c2(NP_001180268) c3(solute carrier family 13 member 3 isoform c precursor) c4(73627, 73628, 73629) c5(73630, 73631, 73632, 73633, 73634, 73635, 73636, 73637, 73638, 73639, 73640, 73641, 73642, 73643, 73644, 73645, 73646, 73647, 73648) c6( ) c7(bL); #c1(SLC13A3) c2(NP_001180269) c3(solute carrier family 13 member 3 isoform d) c4(73649, 73650) c5(73651, 73652, 73653, 73654, 73655, 73656, 73657, 73658, 73659, 73660) c6( ) c7(bL); #c1(SLC13A3) c2(NP_001180271) c3(solute carrier family 13 member 3 isoform e) c4(73668) c5(73669, 73670, 73671, 73672, 73673) c6( ) c7(bL); #c1(SLC13A3) c2(NP_073740) c3(solute carrier family 13 member 3 isoform a precursor) c4(213370, 213371) c5(213372, 213373, 213374, 213375, 213376, 213377, 213378, 213379, 213380, 213381, 213382, 213383, 213384, 213385, 213386, 213387, 213388, 213389, 213390) c6( ) c7(bL); #c1(SLC13A5) c2(NP_001137310) c3(solute carrier family 13 member 5 isoform b) c4(55444) c5(55445, 55446, 55447, 55448, 55449, 55450, 55451, 55452, 55453, 55454) c6( ) c7(aW, lm, m, aT); #c1(SLC13A5) c2(NP_001271439) c3(solute carrier family 13 member 5 isoform d) c4(103960) c5(103961, 103962, 103963, 103964, 103965, 103966) c6( ) c7(aW, lm, m, aT); #c1(SLC13A5) c2(NP_808218) c3(solute carrier family 13 member 5 isoform a) c4(252711) c5(252712, 252713, 252714, 252715, 252716, 252717) c6( ) c7(aW, lm, m, aT); #c1(SLC14A1) c2(NP_001122060) c3(urea transporter 1 isoform 1) c4(46336, 46337, 46338, 46339) c5(46340, 46341, 46342, 46343, 46344, 46345, 46346, 46347, 46348, 46349) c6( ) c7(bM, A, pc, Jr, vj, aw, qd, dV, aj, dL, pm, lf, aHj, eL, q); #c1(SLC14A1) c2(NP_056949) c3(urea transporter 1 isoform 2) c4( ) c5(188176, 188177, 188178, 188179, 188180, 188181) c6( ) c7(bM, A, pc, Jr, vj, aw, qd, dV, aj, dL, pm, lf, aHj, eL, q); #c1(SLC14A1) c2(XP_011524444) c3 (urea transporter 1 isoform X1) c4(319032, 319033) c5(319034) c6( ) c7(bM, A, pc, Jr, vj, aw, qd, dV, aj, dL, pm, lf, aHj, eL, q); #c1(SLC14A1) c2(XP_011524446) c3 (urea transporter 1 isoform X4) c4( ) c5(319035, 319036, 319037, 319038, 319039, 319040) c6( ) c7(bM, A, pc, Jr, vj, aw, qd, dV, aj, dL, pm, lf, aHj, eL, q); #c1(SLC14A2) c2(XP_011524518) c3(urea transporter 2 isoform X1) c4(319073, 319074) c5(319075, 319076, 319077, 319078, 319079, 319080, 319081, 319082, 319083, 319084, 319085) c6( ) c7(bM, T, aC, pf, ea, if, e, boZ, EQ, CH, bB, fd, cL, HH, aBm, aY, Tz, aR, x, bv, AW, aji, u, dV, iv, K, bU, hH, eL, eN, eG, yo, bbC, dH, aW, ih, alR, hl, apn, Ku, Eg, g, eE, eF, cm, mZ, wT, eY, y, jV, eM, iA, wK, dc, blQ, vA, ZT, Gs, U, cj, vy, uJ, azg, k, m, pw, bi, gD, iu, dD, gU, RX, aw, aj, ww, afE, fr, nA, pM, qN, eW, bis, cc, ec, BR, bKk, Nn, gd, Ak, h, dL, ey, ZW, mV, btr, mY, db, on, pl, he, vx, mU, A, mR, zb, fe, aq, pm, fq, op, mX, W, ky, cEz, dB, Fi, anZ, eD, bP, Ei, hL, FL, bD, it, mT, B, qc, bL, fM, eR, bcb, oe, jx, aA, cR, mS, ti, cz, j, v, C, Wa, q, J, o, eA, PC, cF, Rl, mP, WY, jZ, aql, aT, vH, qv, bK, zp, aUQ, rE, alV, ae, af, jf, qw, iU, Y, yL, r, aaE, lo, aDm, HL, bwL, cl, t, ik, bN, ck, Qi, rR, nM, ac, c, bgy, ig, MB, AH, ro, dy, mf, aan, byK, gT, ao, gV, bNo, qb, aZ, aD, iW, ha, mw, bS, oH, jE, eq, mC, ad, Xv, dP, hX, HF, l, F, CJ, Rq, akR, mW, ds, nl, oW, LA, hD, vc, hP, lQ, ji, yh, yF, mu, aeQ, d, lR, tb, hG, ctG, wc, bpO, nh, eH, dN, gl, kD, yy, aF, eZ, Pd, iR, iV, aUC, bz, dr, qa, dR, ek, es, bF, pc, csw, bE, z, anY, be, ei, hE, aAN, X, us); #c1(SLC14A2) c2(XP_011524519) c3(urea transporter 2 isoform X2) c4( ) c5(319086, 319087, 319088, 319089, 319090) c6( ) c7(bM, T, aC, pf, ea, if, e, boZ, EQ, CH, bB, fd, cL, HH, aBm, aY, Tz, aR, x, bv, AW, aji, u, dV, iv, K, bU, hH, eL, eN, eG, yo, bbC, dH, aW, ih, alR, hl, apn, Ku, Eg, g, eE, eF, cm, mZ, wT, eY, y, jV, eM, iA, wK, dc, blQ, vA, ZT, Gs, U, cj, vy, uJ, azg, k, m, pw, bi, gD, iu, dD, gU, RX, aw, aj, ww, afE, fr, nA, pM, qN, eW, biS, cc, ec, BR, bKk, Nn, gd, Ak, h, dL, ey, ZW, mV, btr, mY, db, on, pl, he, vx, mU, A, mR, zb, fe, aq, pm, fq, op, mX, W, ky, cEz, dB, Fi, anZ, eD, bP, Ei, hL, fL, bD, it, mT, B, qc, bL, fM, eR, bcb, oe, jx, aA, cR, mS, ti, cz, j, v, C, Wa, q, J, o, eA, PC, CF, Rl, mP, WY, jZ, aql, aT, vH, qv, bK, zp, aUQ, rE, alV, ae, af, jf, qw, iU, Y, yL, r, aaE, lo, aDm, HL, bwL, cl, t, ik, bN, ck, Qi, rR, nM, ac, c, bgy, ig, MB, AH, ro, dy, mf, aan, byK, gT, ao, gV, bNo, qb, aZ, aD, iW, ha, mw, bS, OH, jE, eq, mC, ad, Xv, dP, hX, HF, l, F, CJ, Rq, akR, mW, ds, nl, oW, LA, hD, vc, hP, lQ, ji, yh, yF, mu, aeQ, d, lR, tb, hG, ctG, wc, bpO, nh, eH, dN, gl, KD, yy, af, eZ, Pd, iR, iV, aUC, bz, dr, qa, dR, ek, es, bF, pc, csw, bE, z, anY, be, ei, hE, aAN, X, us); #c1(SLC15A1) c2(NP_005064) c3(solute carrier family 15 member 1) c4(152538, 152539, 152540) c5(152541, 152542, 152543, 152544, 152545, 152546, 152547, 152548, 152549, 152550, 152551, 152552, 152553, 152554, 152555, 152556, 152557, 152558, 152559) c6( ) c7(Ht, jz, py, vj, baT, fe, ji, iV, eA); #c1(SLC15A2) c2(NP_001139470) c3(solute carrier family 15 member 2 isoform b) c4(59379, 59380) c5(59381, 59382, 59383, 59384, 59385, 59386, 59387, 59388, 59389, 59390, 59391, 59392) c6( ) c7(ao, vj, V, Y); #c1(SLC15A2) c2(NP_066568) c3(solute carrier family 15 member 2 isoform a) c4(207427) c5(207428, 207429, 207430, 207431, 207432, 207433) c6( ) c7(ao, vj, V, Y); #c1(SLC15A4) c2(NP_663623) c3(solute carrier family 15 member 4) c4(241339) c5(241340, 241341, 241342, 241343, 241344, 241345, 241346, 241347, 241348, 241349, 241350, 241351) c6( ) c7(bL, v, eZ, fe); #c1(SLC15A4) c2(XP_011536197) c3(solute carrier family 15 member 4 isoform X1) c4(333492) c5( ) c6( ) c7(bL, v, eZ, fe); #c1(SLC16A1) c2(XP_011540329) c3(monocarboxylate transporter 1 isoform X1) c4(338775, 338776, 338777, 338778, 338779) c5(338780, 338781, 338782, 338783, 338784, 338785, 338786, 338787, 338788, 338789, 338790, 338791, 338792, 338793, 338794, 338795, 338796, 338797, 338798, 338799, 338800, 338801, 338802, 338803, 338804, 338805, 338806, 338807, 338808, 338809, 338810, 338811, 338812, 338813) c6( ) c7(T, dD, fM, cEA, ea, jh, fd, eq, ci, il, eA, ad, cEB, mj, aT, DO, jy, K, hg, fb, ae, d, nO, aW, ff, eH, fe, en, iV, eF, cm, bz, gD, bo, X); #c1(SLC16A10) c2(NP_061063) c3(monocarboxylate transporter 10) c4(199130, 199131) c5(199132, 199133, 199134, 199135, 199136, 199137, 199138, 199139, 199140, 199141, 199142, 199143) c6( ) c7(eL, h, dL, cEC, K, qN, ae, aR, ad); #c1(SLC16A10) c2(XP_006715392) c3(monocarboxylate transporter 10 isoform X2) c4(289509, 289510) c5(289511, 289512, 289513, 289514, 289515, 289516, 289517, 289518, 289519, 289520, 289521, 289522) c6( ) c7(eL, h, dL, cEC, K, qN, ae, aR, ad); #c1(SLC16A10) c2(XP_011533725) c3(monocarboxylate transporter 10 isoform X3) c4(329803, 329804) c5(329805, 329806, 329807, 329808, 329809, 329810, 329811, 329812, 329813, 329814, 329815, 329816) c6( ) c7(eL, h, dL, cEC, K, qN, ae, aR, ad); #c1(SLC16A11) c2(NP_699188) c3(monocarboxylate transporter fl) c4(245828, 245829) c5(245830, 245831, 245832, 245833, 245834, 245835, 245836, 245837, 245838, 245839, 245840, 245841) c6( ) c7(bL, t, m); #c1(SLC16A12) c2(NP_998771) c3(monocarboxylate transporter 12) c4(265894, 265895, 265896, 265897) c5(265898, 265899, 265900, 265901, 265902, 265903, 265904, 265905, 265906, 265907, 265908, 265909, 265910, 265911, 265912, 265913, 265914, 265915, 265916, 265917, 265918, 265919, 265920, 265921) c6( ) c7(aWG, aA, cED, aF, fq, cEE); #c1(SLC16A13) c2(NP_963860) c3(monocarboxylate transporter 13) c4(262444) c5(262445, 262446, 262447, 262448, 262449, 262450) c6( ) c7(bL); #c1(SLC16A2) c2(NP_006508) c3(monocarboxylate transporter 8) c4(165883) c5(165884, 165885, 165886, 165887, 165888, 165889) c6( ) c7(pv, Mo, nM, VX, akx, hr, nf, hz, K, cEC, nA, cb, gT, GR, xt); #c1(SLC16A2) c2(XP_011529317) c3(monocarboxylate transporter 8 isoform X2) c4( ) c5(324745) c6( ) c7(pv, Mo, nM, VX, akx, hr, nf, hz, K, cEC, nA, cb, gT, GR, xt); #c1(SLC16A3) c2(XP_011521909) c3(monocarboxylate transporter 4 isoform X1) c4(316527) c5(316528, 316529, 316530, 316531, 316532, 316533, 316534, 316535, 316536, 316537, 316538, 316539, 316540, 316541, 316542, 316543, 316544, 316545, 316546, 316547, 316548, 316549, 316550, 316551, 316552, 316553, 316554, 316555, 316556, 316557, 316558, 316559, 316560, 316561, 316562, 316563, 316564, 316565, 316566, 316567, 316568, 316569, 316570, 316571, 316572, 316573, 316574, 316575, 316576, 316577, 316578, 316579, 316580, 316581, 316582, 316583, 316584, 316585, 316586, 316587, 316588, 316589, 316590, 316591, 316592, 316593, 316594, 316595, 316596, 316597, 316598, 316599, 316600, 316601, 316602, 316603, 316604, 316605, 316606, 316607, 316608, 316609, 316610, 316611, 316612, 316613, 316614, 316615, 316616, 316617, 316618, 316619, 316620, 316621, 316622, 316623, 316624, 316625, 316626, 316627, 316628, 316629, 316630, 316631, 316632, 316633, 316634, 316635) c6(316636) c7(T, dD, nz, ea, fd, eq, fr, eA, ad, bv, py, aT, K, fb, ae, nO, aW, ff, en, es, iA, lC); #c1(SLC16A6) c2(NP_004685) c3(monocarboxylate transporter 7) c4(148829) c5(148830, 148831, 148832, 148833, 148834, 148835, 148836, 148837, 148838, 148839, 148840, 148841, 148842, 148843) c6( ) c7(dP); #c1(SLC16A6) c2(XP_005257846) c3(monocarboxylate transporter 7 isoform X2) c4(275379) c5(275380, 275381) c6( ) c7(dP); #c1(SLC16A7) c2(XP_005269288) c3(monocarboxylate transporter 2 isoform X1) c4(282534) c5(282535, 282536, 282537, 282538, 282539, 282540, 282541, 282542, 282543, 282544, 282545, 282546, 282547, 282548, 282549, 282550, 282551, 282552, 282553) c6( ) c7(fb, v, bo); #c1(SLC16A7) c2(XP_011537299) c3(monocarboxylate transporter 2 isoform X3) c4(335003) c5(335004, 335005, 335006, 335007, 335008) c6( ) c7(fb, v, bo); #c1(SLC16A8) c2(NP_037488) c3(monocarboxylate transporter 3) c4(175824, 175825) c5(175826, 175827, 175828, 175829, 175830, 175831, 175832, 175833, 175834, 175835, 175836, 175837) c6( ) c7(lC, vj, dD, eq, ea); #c1(SLC16A9) c2(NP_919274) c3(monocarboxylate transporter 9) c4(258396, 258397) c5(258398, 258399, 258400, 258401, 258402, 258403, 258404, 258405, 258406, 258407, 258408, 258409, 258410, 258411, 258412, 258413) c6( ) c7(nD, aj, nn); #c1(SLC17A1) c2(NP_005065) c3(sodium-dependent phosphate transport protein 1) c4(152560, 152561) c5(152562, 152563, 152564, 152565, 152566, 152567, 152568, 152569, 152570, 152571, 152572) c6( ) c7(nD, T, AB, nn, fQ, cEF, AA, af); #c1(SLC17A1) c2(XP_011513122) c3(sodium-dependent phosphate transport protein 1 isoform X3) c4(304916) c5(304917, 304918, 304919, 304920, 304921, 304922, 304923, 304924, 304925, 304926, 304927, 304928, 304929, 304930, 304931) c6( ) c7(nD, T, AB, nn, fQ, cEF, AA, af); #c1(SLC17A2) c2(NP_001273052) c3(sodium-dependent phosphate transport protein 3 isoform a) c4(104260) c5(104261, 104262, 104263, 104264, 104265, 104266) c6( ) c7(nD); #c1(SLC17A2) c2(NP_005826) c3(sodium-dependent phosphate transport protein 3 isoform b) c4(159713) c5(159714, 159715, 159716, 159717, 159718, 159719) c6( ) c7(nD); #c1(SLC17A2) c2(XP_011512529) c3(sodium-dependent phosphate transport protein 3 isoform X4) c4(303185) c5(303186, 303187, 303188, 303189, 303190, 303191) c6( ) c7(nD); #c1(SLC17A3) c2(NP_001091956) c3(sodium-dependent phosphate transport protein 4 isoform a) c4(39011, 39012) c5(39013, 39014, 39015, 39016, 39017, 39018) c6( ) c7(nD, nH, awC, M, cEG, CEH); #c1(SLC17A3) c2(NP_006623) c3(sodium-dependent phosphate transport protein 4 isoform b) c4(166682, 166683) c5(166684, 166685, 166686, 166687, 166688, 166689, 166690, 166691, 166692, 166693, 166694, 166695, 166696, 166697, 166698, 166699, 166700, 166701, 166702, 166703, 166704, 166705) c6( ) c7(nD, nH, awC, M, cEG, cEH); #c1(SLC17A4) c2(NP_005486) c3(probable small intestine urate exporter isoform a) c4(156705) c5(156706, 156707, 156708, 156709, 156710, 156711) c6( ) c7(dD, eq); #c1(SLC17A4) c2(XP_011512519) c3(probable small intestine urate exporter isoform X7) c4(303150) c5(303151, 303152, 303153, 303154, 303155, 303156) c6( ) c7(dD, eq); #c1(SLC17A4) c2(XP_011512521) c3 (probable small intestine urate exporter isoform X9) c4(303157) c5(303158, 303159, 303160, 303161, 303162, 303163) c6( ) c7(dD, eq); #c1(SLC17A5) c2(NP_036566) c3(sialin) c4(174441) c5(174442, 174443, 174444, 174445, 174446, 174447, 174448) c6( ) c7(bM, T, dD, eR, ea, al, jx, aj, v, eq, jE, aY, J, kL, hX, F, alp, oW, h, bBN, K, bzw, cEl, Y, fc, gC, hl, lo, lm, gk, alm, cEJ, ayS, U, Lq, cP, hE, B, vi); #c1(SLC17A5) c2(XP_011534052) c3(sialin isoform X3) c4(330226) c5(330227) c6( ) c7(bM, T, dD, eR, ea, al, jx, aj, v, eq, jE, aY, J, kL, hX, F, alp, oW, h, bBN, K, bzw, cEl, Y, fc, gC, hl, lo, lm, gk, alm, cEJ, ayS, U, Lq, cP, hE, B, vi); #c1(SLC17A5) c2(XP_011534053) c3(sialin isoform X4) c4( ) c5(330228, 330229, 330230, 330231, 330232) c6( ) c7(bM, T, dD, eR, ea, al, jx, aj, v, eq, jE, aY, J, kL, hX, F, alp, oW, h, bBN, K, bzw, cEl, Y, fc, gC, hl, lo, lm, gk, alm, cEJ, ayS, U, Lq, cP, hE, B, vi); #c1(SLC17AG) c2(NP_065079) c3(vesicular glutamate transporter 2) c4(203721, 203722) c5(203723, 203724, 203725, 203726, 203727, 203728, 203729, 203730, 203731, 203732, 203733, 203734) c6( ) c7(ot, c, ha, bO, bk, an, xt, ad); #c1(SLC17A7) c2(NP_064705) c3(vesicular glutamate transporter 1) c4(203488, 203489) c5(203490, 203491, 203492, 203493, 203494, 203495, 203496, 203497, 203498, 203499, 203500, 203501) c6( ) c7(ot, ha, bO, bK, K, xt); #c1(SLC17A8) c2(NP_001138760) c3(vesicular glutamate transporter 3 isoform 2) c4(57580) c5(57581, 57582, 57583, 57584, 57585, 57586, 57587) c6( ) c7(Bh, mG, ff, en, cEK); #c1(SLC17A8) c2(NP_647480) c3(vesicular glutamate transporter 3 isoform 1) c4(238686) c5(238687, 238688, 238689, 238690, 238691, 238692) c6( ) c7(Bh, mG, ff, en, cEK); #c1(SLC18A1) c2(XP_011542926) c3(chromaffin granule amine transporter isoform X1) c4(341532, 341533) c5(341534, 341535, 341536, 341537, 341538, 341539, 341540, 341541, 341542, 341543, 341544) c6( ) c7(bxy, T, cF, d, eP, aW, U, aT, uJ, bS, bK, ct, Z, oi, cm, bt, eA, VB, hq); #c1(SLC18A1) c2(XP_011542928) c3(chromaffin granule amine transporter isoform X2) c4(341545) c5(341546, 341547, 341548, 341549, 341550, 341551, 341552, 341553, 341554, 341555) c6( ) c7(bxy, T, cF, d, eP, aW, U, aT, uJ, bS, bK, ct, Z, oi, cm, bt, eA, VB, hq); #c1(SLC18A1) c2(XP_011542930) c3(chromaffin granule amine transporter isoform X3) c4(341556) c5(341557, 341558, 341559, 341560, 341561, 341562, 341563, 341564, 341565, 341566, 341567) c6( ) c7(bxy, T, cF, d, eP, aW, U, aT, uJ, bS, bK, ct, Z, oi, cm, bt, eA, VB, hq); #c1(SLC18A2) c2(NP_003045) c3(synaptic vesicular amine transporter) c4(134641) c5(134642, 134643, 134644, 134645, 134646, 134647) c6( ) c7(T, bO, n, aj, bS, gf, ct, Fb, VV, aR, ad, J, HF, cF, aji, HC, bK, K, Wm, an, oi, ae, af, alL, mu, A, Gv, HL, Z, jb, FT, hq, iq, ok, rR, cA, Kv, Gs, Ei, QF, sv, uJ, anx, lB, VB); #c1(SLC18A3) c2(NP_003046) c3(vesicular acetylcholine transporter) c4(134648) c5(134649, 134650, 134651, 134652, 134653, 134654) c6( ) c7(rt, bM, jo, aNR, af, Hr); #c1(SLC19A1) c2(NP_001192135) c3(folate transporter 1 isoform 2) c4(81933, 81934, 81935, 81936) c5(81937, 81938, 81939, 81940, 81941, 81942, 81943, 81944, 81945, 81946, 81947, 81948, 81949, 81950, 81951) c6( ) c7(ng, T, gU, fM, fV, iW, jh, aA, sM, cz, cL, bB, j, ci, Gn, q, aR, o, ad, x, fU, av, Na, Bj, fS, gd, aT, Nc, dL, bK, K, eL, iL, eu, s, aoX, hG, aW, vj, ff, eH, r, Ll, Lk, af, en, g, eF, f, iK, fq, bz, iV, bN, ck, ac, eM, bP, gY, pE, lp, wE, bwp, bm, at, k, ij, bD, y, rA, X, us); #c1(SLC19A1) c2(NP_001192136) c3(folate transporter 1 isoform 3) c4(81952) c5(81953, 81954, 81955, 81956, 81957, 81958) c6( ) c7(ng, T, gU, fM, fV, iW, jh, aA, sM, cz, cL, bB, j, ci, Gn, q, aR, o, ad, x, fU, av, Na, Bj, fS, gd, aT, Nc, dL, bK, K, eL, iL, eu, s, aoX, hG, aW, vj, ff, eH, r, Ll, Lk, aF, en, g, eF, f, ik, fq, bz, iV, bN, ck, ac, eM, bP, gY, pE, lp, wE, bwp, bm, at, k, ij, bD, y, rA, X, us); #c1(SLC19A1) c2(XP_011528000) c3(folate transporter 1 isoform X3) c4( ) c5(323088) c6( ) c7(ng, T, gU, fM, fV, iW, jh, aA, sM, cz, cL, bB, j, ci, Gn, q, aR, o, ad, x, fU, av, Na, Bj, fS, gd, aT, Nc, dL, bK, K, eL, iL, eu, s, aoX, hG, aW, vj, ff, eH, r, Ll, Lk, af, en, g, eF, f, ik, fq, bz, iV, bN, ck, ac, eM, bP, gY, pE, lp, wE, bwp, bm, at, k, ij, bD, y, rA, X, us); #c1(SLC19A1) c2(XP_011528007) c3(folate transporter 1 isoform X5) c4( ) c5(323089, 323090, 323091, 323092, 323093, 323094) c6( ) c7(ng, T, gU, fM, fV, iW, jh, aA, sM, cz, cL, bB, j, ci, Gn, q, aR, o, ad, x, fU, av, Na, Bj, fS, gd, aT, Nc, dL, bK, K, eL, iL, eu, s, aoX, hG, aW, vj, ff, eH, r, Ll, Lk, aF, en, g, eF, f, ik, fq, bz, iV, bN, ck, ac, eM, bP, gY, pE, lp, wE, bwp, bm, at, k, ij, bD, y, rA, X, us); #c1(SLC19A1) c2(XP_011528009) c3(folate transporter 1 isoform X7) c4( ) c5(323095, 323096, 323097, 323098) c6( ) c7(ng, T, gU, fM, fV, iW, jh, aA, sM, cz, cL, bB, j, ci, Gn, q, aR, o, ad, x, fU, av, Na, Bj, fS, gd, aT, Nc, dL, bK, K, eL, iL, eu, s, aoX, hG, aW, vj, ff, eH, r, Ll, Lk, aF, en, g, eF, f, ik, fq, bz, iV, bN, ck, ac, eM, bP, gY, pE, lp, wE, bwp, bm, at, k, ij, bD, y, rA, X, us); #c1(SLC19A1) c2(XP_011528010) c3(folate transporter 1 isoform X1) c4(323099) c5(323100, 323101, 323102, 323103, 323104) c6( ) c7(ng, T, gU, fM, fV, iW, jh, aA, sM, cz, cL, bB, j, ci, Gn, q, aR, o, ad, x, fU, av, Na, Bj, fS, gd, aT, Nc, dL, bK, K, eL, iL, eu, s, aoX, hG, aW, vj, ff, eH, r, Ll, Lk, aF, en, g, eF, f, ik, fq, bz, iV, bN, ck, ac, eM, bP, gY, pE, lp, wE, bwp, bm, at, k, ij, bD, y, rA, X, us); #c1(SLC19A1) c2(XP_011528012) c3(folate transporter 1 isoform X8) c4(323105, 323106) c5(323107, 323108, 323109, 323110, 323111, 323112, 323113, 323114, 323115, 323116, 323117) c6( ) c7(ng, T, gU, fM, fV, iW, jh, aA, sM, cz, cL, bB, j, ci, Gn, q, aR, o, ad, x, fU, av, Na, Bj, fS, gd, aT, Nc, dL, bK, K, eL, iL, eu, s, aoX, hG, aW, vj, ff, eH, r, Ll, Lk, af, en, g, eF, f, ik, fq, bz, iV, bN, ck, ac, eM, bP, gY, pE, lp, wE, bwp, bm, at, k, ij, bD, y, rA, X, us); #c1(SLC19A2) c2(NP_008927) c3(thiamine transporter 1) c4(169592, 169593) c5(169594, 169595, 169596, 169597, 169598, 169599, 169600, 169601, 169602, 169603, 169604, 169605) c6( ) c7(bL, fU, T, cEL, Gm, cCU, UP, iU, se, aW, sq, oW, aT, cz, aj, cEM, J, bA); #c1(SLC19A2) c2(XP_011507379) c3(thiamine transporter 1 isoform X2) c4(297235) c5(297236, 297237, 297238, 297239, 297240, 297241) c6( ) c7(bL, fU, T, cEL, Gm, cCU, UP, iU, se, aW, sq, oW, aT, cz, aj, cEM, J, bA); #c1(SLC19A3) c2(XP_005246931) c3 (thiamine transporter 2 isoform X4) c4(267515) c5(267516, 267517, 267518, 267519, 267520, 267521) c6( ) c7(uB, fU, T, cCU, hb, aW, cEN, aT, bcD, aA, aF, K, ajU); #c1(SLC19A3) c2(XP_006712842) c3(thiamine transporter 2 isoform X3) c4(286894) c5( ) c6( ) c7(uB, fU, T, cCU, hb, aW, cEN, aT, bcD, aA, aF, K, ajU); #c1(SLC19A3) c2(XP_011510233) c3(thiamine transporter 2 isoform X2) c4(300800, 300801, 300802) c5(300803, 300804, 300805, 300806, 300807) c6( ) c7(uB, fU, T, cCU, hb, aW, cEN, aT, bcD, aA, aF, K, ajU); #c1(SLC19A3) c2(XP_011510235) c3(thiamine transporter 2 isoform X1) c4( ) c5(300808, 300809, 300810, 300811, 300812, 300813, 300814, 300815, 300816, 300817, 300818, 300819, 300820, 300821, 300822, 300823, 300824, 300825, 300826, 300827, 300828, 300829, 300830) c6( ) c7(uB, fU, T, cCU, hb, aW, cEN, aT, bcD, aA, aF, K, ajU); #c1(SLC1A1) c2(NP_004161) c3(excitatory amino acid transporter 3) c4(143961) c5(143962, 143963, 143964, 143965, 143966, 143967) c6( ) c7(cmC, cEO, ha, bO, GC, cEP, nA, il, Fc, V, aR, dP, hb, cy, bK, K, rE, he, GZ, ahe, bN, c, ig, pk, qc); #c1(SLC1A1) c2(XP_011516311) c3(excitatory amino acid transporter 3 isoform X3) c4(309523) c5( ) c6( ) c7(cmC, cED, ha, bO, GC, cEP, nA, il, Fc, V, aR, dP, hb, cy, bK, K, rE, he, GZ, ahe, bN, c, ig, pk, qc); #c1(SLC1A1) c2(XP_011516312) c3(excitatory amino acid transporter 3 isoform X4) c4( ) c5(309524, 309525, 309526, 309527, 309528, 309529) c6( ) c7(cmC, cEO, ha, bO, GC, cEP, nA, il, Fc, V, aR, dP, hb, cy, bK, K, rE, he, GZ, ahe, bN, c, ig, pk, qc); #c1(SLC1A2) c2(NP_001182657) c3(excitatory amino acid transporter 2 isoform 2) c4( ) c5(75133, 75134, 75135, 75136, 75137, 75138, 75139, 75140) c6( ) c7(T, bmQ, Ol, dM, yY, ha, ex, l, EQ, aA, n, ti, bO, Vd, asF, PJ, il, V, eA, aR, KL, Gt, aV, hb, HC, h, eV, bK, K, jj, an, he, af, nP, aCt, zD, aPf, cu, aF, as, bN, hq, ek, rR, c, bQC, Gs, cEQ, U, uJ, Da, mk, KS); #c1(SLC1A2) c2(NP_004162) c3(excitatory amino acid transporter 2 isoform 1) c4(143968) c5(143969, 143970, 143971, 143972, 143973, 143974) c6( ) c7(T, bmQ, Ol, dM, yY, ha, eX, l, ER, aA, n, ti, bO, Vd, asF, PJ, il, V, eA, aR, KL, Gt, aV, hb, HC, h, eV, bK, K, jj, an, he, af, nP, aCt, zD, aPf, cu, aF, as, bN, hq, ek, rR, c, bQC, Gs, cEQ, U, uJ, Da, mk, kS); #c1(SLC1A2) c2(XP_005253124) c3(excitatory amino acid transporter 2 isoform X2) c4(272172) c5(272173, 272174) c6( ) c7(T, bm, Ol, dM, yY, ha, eX, l, EQ, aA, n, ti, bO, Vd, asF, PJ, il, V, eA, aR, KL, Gt, aV, hb, HC, h, eV, bK, K, jj, an, he, af, nP, aCt, zD, aPf, cu, aF, as, bN, hq, ek, rR, c, bQC, Gs, cEQ, U, uJ, Da, mk, kS); #c1(SLC1A3) c2(NP_001160167) c3(excitatory amino acid transporter 1 isoform 2) c4(65181) c5(65182, 65183, 65184, 65185, 65186, 65187) c6( ) c7(cmC, cZ, dt, cER, ha, bO, axA, dG, uu, V, WY, h, ey, K, dd, akr, yL, ru, ban, fq, bN, hq, aki, ayo); #c1(SLC1A3) c2(NP_001160168) c3(excitatory amino acid transporter 1 isoform 3) c4(65188) c5(65189, 65190, 65191, 65192, 65193, 65194) c6( ) c7(cmC, cZ, dt, cER, ha, bO, axA, dG, uu, V, WY, h, ey, K, dd, akr, yL, rU, ban, fq, bN, hq, aki, ayo); #c1(SLC1A3) c2(NP_001276868) c3(excitatory amino acid transporter 1 isoform 4) c4(108537) c5(108538, 108539, 108540, 108541, 108542, 108543) c6( ) c7(cmC, cZ, dt, cER, ha, bO, axA, dG, uu, V, WY, h, ey, K, dd, akr, yL, rU, ban, fq, bN, hq, aki, ayo); #c1(SLC1A3) c2(NP_001276869) c3(excitatory amino acid transporter 1 isoform 5) c4(108544, 108545) c5(108546, 108547, 108548, 108549, 108550, 108551, 108552, 108553, 108554, 108555, 108556, 108557, 108558, 108559, 108560, 108561) c6( ) c7(cmC, cZ, dt, cER, ha, bO, axA, dG, uu, V, WY, h, ey, K, dd, akr, yL, rU, ban, fq, bN, hq, aki, ayo); #c1(SLC1A3) c2(XP_005248399) c3(excitatory amino acid transporter 1 isoform X1) c4(268373) c5(268374, 268375, 268376, 268377, 268378, 268379) c6( ) c7(cmC, cZ, dt, cER, ha, bO, axA, dG, uu, V, WY, h, ey, K, dd, akr, yL, rU, ban, fq, bN, hq, aki, ayo); #c1(SLC1A4) c2(NP_00180422) c3(neutral amino acid transporter A isoform 2 precursor) c4(74038) c5(74039, 74040, 74041, 74042, 74043, 74044) c6( ) c7(GC, vj, ac, K); #c1(SLC1A4) c2(NP_003029) c3(neutral amino acid transporter A isoform 1) c4(134559, 134560) c5(134561, 134562, 134563, 134564, 134565, 134566, 134567, 134568, 134569, 134570, 134571, 134572) c6( ) c7(GC, vj, ac, K); #c1(SLC1A5) c2(NP_001138616) c3(neutral amino acid transporter B(O) isoform 2) c4( ) c5(57191, 57192, 57193, 57194, 57195) c6( ) c7(T, nl, if, qN, ad, px, cy, beo, B, Y, ff, aq, en, g, uC, bz, eM, bC, bt, wS, ao, cn); #c1(SLC1A5) c2(NP_001138617) c3(neutral amino acid transporter B(O) isoform 3) c4( ) c5(57196, 57197, 57198, 57199, 57200, 57201) c6( ) c7(T, nl, if, qN, ad, pX, cy, beo, B, Y, ff, aq, en, g, uC, bz, eM, bC, bt, wS, ao, cn); #c1(SLC1A5) c2(NP_005619) c3(neutral amino acid transporter B(O) isoform 1) c4(157981, 157982, 157983, 157984) c5(157985, 157986, 157987, 157988, 157989, 157990, 157991, 157992, 157993, 157994, 157995, 157996, 157997) c6( ) c7(T, nl, if, qN, ad, pX, cy, beo, B, Y, ff, aq, en, g, uC, bz, eM, bC, bt, wS, ao, cn); #c1(SLC1A6) c2(NP_001259017) c3(excitatory amino acid transporter 4 isoform 1) c4( ) c5(96939, 96940, 96941, 96942, 96943, 96944, 96945, 96946, 96947, 96948, 96949, 96950) c6( ) c7(b0, vj, BC, bK, kH); #c1(SLC1A6) c2(XP_006722906) c3(excitatory amino acid transporter 4 isoform X1) c4(294940, 294941, 294942, 294943) c5(294944, 294945, 294946, 294947, 294948, 294949, 294950, 294951, 294952, 294953, 294954, 294955) c6( ) c7(b0, vj, BC, bK, kH); #c1(SLC20A1) c2(NP_005406) c3(sodium-dependent phosphate transporter 1) c4(155773, 155774) c5(155775, 155776, 155777, 155778, 155779, 155780) c6( ) c7(Ly, lT, acz, aYc, bBS, ci, AO, biN, dP, fU, ib, RN, oW, aT, ey, yo, avP, wr, s, aW, CM, wU, OG, hR, lS, im, gT, VK); #c1(SLC20A1) c2(XP_011509988) c3(sodium-dependent phosphate transporter 1 isoform X1) c4( ) c5(300598, 300599, 300600, 300601, 300602, 300603) c6( ) c7(Ly, lT, acz, aYc, bBS, ci, AO, biN, dP, fU, ib, RN, oW, aT, ey, yo, avP, wr, s, aW, CM, wU, OG, hR, lS, im, gT, VK); #c1(SLC20A2) c2(XP_005273670) c3(sodium-dependent phosphate transporter 2 isoform X1) c4(284774) c5(284775, 284776, 284777, 284778, 284779, 284780, 284781, 284782, 284783, 284784, 284785, 284786, 284787, 284788, 284789, 284790, 284791, 284792, 284793, 284794, 284795, 284796, 284797, 284798) c6( ) c7(fU, PW, ib, aYc, aU, an); #c1(SLC20A2) c2(XP_005273672) c3(sodium-dependent phosphate transporter 2 isoform X2) c4(284799, 284800, 284801, 284802) c5( ) c6( ) c7(fU, PW, ib, aYc, al, an); #c1(SLC20A2) c2(XP_006716453) c3(sodium-dependent phosphate transporter 2 isoform X3) c4( ) c5(290315, 290316, 290317, 290318, 290319, 290320, 290321, 290322, 290323, 290324, 290325, 290326, 290327, 290328) c6( ) c7(fU, PW, ib, aYc, aU, an); #c1(SLC22A1) c2(NP_003048) c3(solute carrier family 22 member 1 isoform a) c4(134655) c5(134656, 134657, 134658, 134659, 134660, 134661) c6( ) c7(bL, dD, fM, bkx, aZ, ea, if, ht, jx, jh, cz, aj, v, eq, ci, eh, jw, J, ad, jz, F, aE, Do, aMb, qp, dV, aT, K, jo, bq, hu, bc, ls, kl, B, iU, vj, fe, aF, hJ, ck, eM, eD, hR, E, y, X); #c1(SLC22A11) c2(NP_060954) c3(solute carrier family 22 member 11) c4(198871) c5(198872, 198873, 198874, 198875, 198876, 198877) c6( ) c7(nD, nH, nn, h, aY, bA); #c1(SLC22A11) c2(XP_011543469) c3(solute carrier family 22 member 11 isoform X2) c4( ) c5(342370) c6( ) c7(nD, nH, nn, h, aY, bA); #c1(SLC22A12) c2(NP_001263255) c3(solute carrier family 22 member 12 isoform c) c4(97101) c5( ) c6( ) c7(nD, nH, nn, E, cES, dy, cgP, ckw, ei); #c1(SLC22A12) c2(NP_001263256) c3(solute carrier family 22 member 12 isoform d) c4(97102) c5(97103, 97104, 97105, 97106, 97107, 97108) c6( ) c7(nD, nH, nn, E, cES, dy, cgP, ckw, ei); #c1(SLC22A12) c2(NP_653186) c3(solute carrier family 22 member 12 isoform a) c4(238971) c5(238972, 238973, 238974, 238975, 238976, 238977) c6( ) c7(nD, nH, nn, E, cES, dy, cgP, ckw, ei); #c1(SLC22A12) c2(NP_700357) c3(solute carrier family 22 member 12 isoform b) c4( ) c5(245919, 245920, 245921, 245922, 245923, 245924) c6( ) c7(nD, nH, nn, E, cES, dy, cgP, ckw, ei); #c1(SLC22A12) c2(XP_006718494) c3(solute carrier family 22 member 12 isoform X2) c4( ) c5(291887) c6( ) c7(nD, nH, nn, E, cES, dy, cgP, ckw, ei); #c1(SLC22A13) c2(NP_004247) c3(solute carrier family 22 member 13) c4(144581, 144582) c5(144583, 144584, 144585, 144586, 144587, 144588, 144589, 144590, 144591, 144592, 144593, 144594) c6( ) c7(es, K); #c1(SLC22A14) c2(NP_004794) c3(solute carrier family 22 member (4) c4(149912) c5(149913, 149914, 149915, 149916, 149917, 149918, 149919, 149920) c6( ) c7(FE); #c1(SLC22A14) c2(XP_006713479) c3(solute carrier family 22 member 14 isoform X2) c4( ) c5(287167, 287168) c6( ) c7(FE); #c1(SLC22A16) c2(NP_149116) c3(solute carrier family 22 member 16) c4(227259, 227260, 227261) c5(227262, 227263, 227264, 227265, 227266, 227267, 227268, 227269, 227270, 227271, 227272, 227273) c6( ) c7(ck, aW, hL, aT, oi, B); #c1(SLC22A16) c2(XP_005267241) c3(solute carrier family 22 member 16 isoform X9) c4(280836) c5(280837, 280838, 280839, 280840, 280841) c6( ) c7(ck, aW, hL, al, oi, B); #c1(SLC22A16) c2(XP_011534506) c3(solute carrier family 22 member 16 isoform X1) c4(330974, 330975) c5( ) c6( ) c7(ck, aW, hL, aT, oi, B): #c1(SLC22A16) c2(XP_011534507) c3(solute carrier family 22 member 16 isoform X2) c4(330976, 330977) c5(330978, 330979, 330980, 330981, 330982, 330983, 330984, 330985, 330986, 330987, 330988, 330989) c6( ) c7(ck, aW, hL, al, oi, B); #c1(SLC22A16) c2(XP_011534511) c3(solute carrier family 22 member 16 isoform X6) c4( ) c5(330990) c6( ) c7(ck, aW, hL, aT, oi, B); #c1(SLC22A16) c2(XP_011534512) c3(solute carrier family 22 member 16 isoform X7) c4( ) c5(330991, 330992, 330993, 330994, 330995, 330996, 330997, 330998, 330999, 331000, 331001, 331002, 331003, 331004, 331005, 331006, 331007, 331008) c6( ) c7(ck, aW, hL, aT, oi, B); #c1(SLC22A17) c2(NP_057693) c3(solute carrier family 22 member 17 isoform b) c4(192384) c5(192385, 192386, 192387, 192388, 192389, 192390) c6( ) c7(B, kP, iU, ci, hu, it, eA); #c1(SLC22A17) c2(NP_065105) c3(solute carrier family 22 member 17 isoform a) c4(203948) c5(203949, 203950, 203951, 203952, 203953, 203954, 203955, 203956, 203957, 203958) c6( ) c7(B, kP, iU, ci, hu, it, eA); #c1(SLC22A18) c2(NP_899056) c3(solute carrier family 22 member 18) c4(257900, 257901) c5(257902, 257903, 257904, 257905, 257906, 257907, 257908, 257909, 257910, 257911, 257912) c6( ) c7(T, akc, pf, if, ml, qN, cET, eA, er, bv, av, dE, dV, aT, aPe, aW, ff, LZ, en, g, bz, iO, el, im, fy, cE, B); #c1(SLC22A18) c2(XP_011518442) c3(solute carrier family 22 member 18 isoform X2) c4( ) c5(312503, 312504, 312505) c6( ) c7(T, akc, pf, if, ml, qN, cET, eA, er, bv, av, dE, dV, aT, aPe, aW, ff, LZ, en, g, bz, iO, el, im, fy, cE, B); #c1(SLC22A18) c2(XP_011518444) c3(solute carrier family 22 member 18 isoform X7) c4( ) c5(312506, 312507, 312508, 312509, 312510) c6( ) c7(T, akc, pf, if, ml, qN, cET, eA, er, bv, av, dE, dV, aT, aPe, aW, ff, LZ, en, g, bz, iO, el, im, fy, cE, B); #c1(SLC22A18AS) c2(NP_001289791) c3(beckwith-Wiedemann syndrome chromosomal region 1 candidate gene B protein isoform b) c4(114626) c5(114627, 114628, 114629, 114630, 114631, 114632, 114633, 114634, 114635, 114636) c6( ) c7(cm, ml); #c1(SLC22A18AS) c2(NP_009036) c3(beckwith-Wiedemann syndrome chromosomal region 1 candidate gene B protein isoform a) c4(170349) c5(170350, 170351, 170352, 170353, 170354, 170355) c6( ) c7(cm, ml); #c1(SLC22A2) c2(NP_003049) c3(solute carrier family 22 member 2) c4(134662, 134663) c5(134664, 134665, 134666, 134667, 134668, 134669, 134670, 134671, 134672, 134673, 134674, 134675) c6( ) c7(bL, T, fM, eR, ex, RC, cz, fd, jh, aj, j, aY, jw, nH, eV, jo, bq, ls, A, iU, zL, t, fq, fP, E, dy, m, bt); #c1(SLC22A23) c2(NP_001273385) c3(solute carrier family 22 member 23 isoform c) c4( ) c5(104812, 104813, 104814, 104815, 104816, 104817) c6( ) c7(dP, fe, iV); #c1(SLC22A23) c2(NP_056297) c3(solute carrier family 22 member 23 isoform a) c4(187005) c5(187006, 187007, 187008, 187009, 187010, 187011, 187012, 187013) c6( ) c7(dP, fe, iV); #c1(SLC22A23) c2(NP_068764) c3(solute carrier family 22 member 23 isoform b) c4(210266) c5(210267, 210268, 210269, 210270, 210271, 210272, 210273, 210274) c6( ) c7(dP, fe, iV); #c1(SLC22A23) c2(XP_005249342) c3(solute carrier family 22 member 23 isoform X2) c4(269239) c5( ) c6( ) c7(dP, fe, iV); #c1(SLC22A24) c2(NP_001129978) c3(solute carrier family 22 member 24 isoform 3) c4(52348) c5(52349, 52350, 52351, 52352, 52353, 52354) c6( ) c7(E); #c1(SLC22A24) c2(NP_775857) c3(solute carrier family 22 member 24 isoform 2) c4(250493) c5(250494, 250495, 250496, 250497, 250498, 250499) c6( ) c7(E); #c1(SLC22A3) c2(NP_068812) c3(solute carrier family 22 member 3) c4(210771, 210772) c5(210773, 210774, 210775, 210776, 210777, 210778, 210779, 210780, 210781, 210782, 210783, 210784) c6( ) c7(Mb, T, aC, wu, KY, aA, cz, n, bB, wV, j, ci, fr, AO, q, ad, eA, iu, asG, Bl, F, hd, aE, jz, Pl, aT, rO, dL, K, wm, rE, nJ, ae, eL, A, d, UL, vT, Hl, aW, aeC, Y, eH, AQ, nO, r, nY, gl, Am, aq, kp, aF, g, cl, hJ, t, eF, bz, FT, LB, ok, kP, aDX, nB, bE, eM, eD, iA, X, np, Hb, m, gn, adg, lB, it, B, cn, qc); #c1(SLC22A4) c2(NP_003050) c3(solute carrier family 22 member 4) c4(134676) c5(134677, 134678, 134679, 134680, 134681, 134682) c6( ) c7(Dw, aFc, cz, wV, ai, q, wD, xE, cy, Ds, hH, Xl, fb, cEU, A, cEV, cr, ge, r, eZ, fe, hp, iV, cm, aNF, ck, aDX, WP, wR, fL, y, bt, xb); #c1(SLC22A5) c2(NP_003051) c3(solute carrier family 22 member 5) c4(134683) c5(134684, 134685, 134686, 134687, 134688, 134689) c6( ) c7(bM, T, jv, Dw, ra, qb, ol, ew, aA, cz, rf, wV, bS, j, v, bX, Eh, cd, wD, cc, oj, aT, Ds, vc, K, hH, fb, HO, mu, A, aW, cEV, cr, ge, r, fe, aF, aot, iV, t, uC, bw, aDX, bo, mx, m, pw, du, y, xb, gq); #c1(SLC22A6) c2(NP_004781) c3(solute carrier family 22 member 6 isoform a) c4(149576) c5(149577, 149578, 149579, 149580, 149581, 149582) c6( ) c7(cz, h, dy, lS, bK, iV, aY); #c1(SLC22A6) c2(NP_695008) c3(solute carrier family 22 member 6 isoform b) c4(245644, 245645) c5(245646, 245647, 245648, 245649, 245650, 245651, 245652, 245653, 245654, 245655, 245656, 245657) c6( ) c7(cz, h, dy, lS, bK, iV, aY); #c1(SLC22A6) c2(NP_695009) c3(solute carrier family 22 member 6 isoform c) c4(245658) c5(245659, 245660, 245661, 245662, 245663, 245664) c6( ) c7(cz, h, dy, lS, bk, iV, aY); #c1(SLC22A6) c2(NP_695010) c3(solute carrier family 22 member 6 isoform d) c4(245665) c5(245666, 245667, 245668, 245669, 245670, 245671) c6( ) c7(cz, h, dy, lS, bK, iV, aY); #c1(SLC22A7) c2(NP_006663) c3(solute carrier family 22 member 7 isoform a) c4(166999, 167000) c5(167001, 167002, 167003, 167004, 167005, 167006, 167007, 167008, 167009, 167010, 167011, 167012) c6( ) c7(hJ, hC, B, eM); #c1(SLC22A7) c2(NP_696961) c3(solute carrier family 22 member 7 isoform b) c4(245703) c5(245704, 245705, 245706, 245707, 245708, 245709) c6( ) c7(hJ, hC, B, eM); #c1(SLC22A7) c2(XP_011512559) c3(solute carrier family 22 member 7 isoform X2) c4(303454) c5( ) c6( ) c7(hJ, hC, B, eM); #c1(SLC22A7) c2(XP_011512563) c3(solute carrier family 22 member 7 isoform X8) c4(303455, 303456) c5( ) c6( ) c7(hJ, hC, B, eM); #c1(SLC22A7) c2(XP_011512564) c3(solute carrier family 22 member 7 isoform X9) c4( ) c5(303457, 303458, 303459, 303460, 303461, 303462, 303463, 303464, 303465, 303466, 303467, 303468) c6( ) c7(hJ, hC, B, eM); #c1(SLC22A7) c2(XP_011512565) c3(solute carrier family 22 member 7 isoform X10) c4( ) c5(303469, 303470, 303471, 303472, 303473, 303474) c6( ) c7(hJ, hC, B, eM); #c1(SLC22A8) c2(NP_001171661) c3(solute carrier family 22 member 8 isoform 1) c4(71203, 71204) c5(71205, 71206, 71207, 71208, 71209, 71210, 71211, 71212, 71213, 71214, 71215, 71216, 71217, 71218, 71219, 71220, 71221) c6( ) c7(ds, cz, lo, dy, yV, bX); #c1(SLC22A8) c2(NP_001171662) c3(solute carrier family 22 member 8 isoform 2) c4(71222) c5(71223, 71224, 71225, 71226, 71227, 71228) c6( ) c7(ds, cz, lo, dy, yV, bX); #c1(SLC22A8) c2(XP_011543666) c3(solute carrier family 22 member 8 isoform X1) c4(342772) c5(342773, 342774, 342775, 342776, 342777) c6( ) c7(ds, cz, lo, dy, yV, bX); #c1(SLC23A1) c2(NP_005838) c3(solute carrier family 23 member 1 isoform a) c4(159791) c5( ) c6( ) c7(T, iV, fR, sl, s, dH, iW, hl, jh, aA, uo, aq, ut, dL, at, lQ, aDE, K, hJ, aF, B); #c1(SLC23A1) c2(NP_689898) c3(solute carrier family 23 member 1 isoform b) c4(244290, 244291) c5(244292, 244293, 244294, 244295, 244296) c6( ) c7(T, iV, fR, sQ, s, dH, iW, hl, jh, aA, uo, aq, ut, dL, at, lQ, aDE, K, hJ, aF, B); #c1(SLC23A1) c2(XP_005272206) c3(solute carrier family 23 member 1 isoform X2) c4( ) c5(283758, 283759, 283760, 283761, 283762, 283763) c6( ) c7(T, iV, fR, sQ, s, dH, iW, hl, jh, aA, uo, aq, ut, dL, at, lQ, aDE, K, hJ, aF, B); #c1(SLC23A1) c2(XP_011542067) c3(solute carrier family 23 member 1 isoform X1) c4( ) c5(340592, 340593, 340594, 340595, 340596, 340597) c6( ) c7(T, iV, FR, sl, s, dH, iW, hl, jh, aA, uo, aq, ut, dL, at, lQ, aDE, K, hJ, aF, B); #c1(SLC23A1) c2(XP_011542070) c3(solute carrier family 23 member 1 isoform X6) c4(340598) c5( ) c6( ) c7(T, iV, fR, s, s, dH, iW, hl, jh, aA, uo, aq, ut, dL, at, lQ, aDE, K, hJ, aF, B); #c1(SLC23A1) c2(XP_011542071) c3(solute carrier family 23 member 1 isoform X7) c4( ) c5(340599, 340600, 340601, 340602, 340603, 340604, 340605, 340606) c6( ) c7(T, iV, FR, sQ, s, dH, iW, hl, jh, aA, uo, aq, ut, dL, at, lQ, aDE, K, hJ, aF, B); #c1(SLC23A2) c2(NP_005107) c3(solute carrier family 23 member 2) c4(152943, 152944, 152945) c5(152946, 152947, 152948, 152949, 152950, 152951, 152952, 152953, 152954, 152955) c6( ) c7(s, iW, e, hl, jh, aA, at, dL, aF, K, aDE, hJ, cm, nJ); #c1(SLC23A2) c2(XP_005260961) c3(solute carrier family 23 member 2 isoform X2) c4( ) c5(277018, 277019, 277020, 277021, 277022, 277023, 277024, 277025, 277026, 277027, 277028, 277029) c6( ) c7(s, iW, e, hl, jh, aA, at, dl, aF, K, aDE, hJ, cm, nJ); #c1(SLC24A1) c2(NP_004718) c3(sodium/potassium/calcium exchanger 1 isoform 1) c4(149058) c5(149059, 149060, 149061, 149062, 149063, 149064) c6( ) c7(dd, cEW, lR, cz); #c1(SLC24A1) c2(XP_011520522) c3(sodium/potassium/calcium exchanger 1 isoform X3) c4(315450) c5(315451, 315452, 315453, 315454, 315455, 315456, 315457, 315458, 315459, 315460) c6( ) c7(dd, cEW, lR, CZ); #c1(SLC24A1) c2(XP_011520523) c3(sodium/potassium/calcium exchanger 1 isoform X2) c4(315461) c5(315462, 315463, 315464, 315465, 315466, 315467) c6( ) c7(dd, cEW, lR, cZ); #c1(SLC24A2) c2(XP_005251482) c3(sodium/potassium/calcium exchanger 2 isoform X1) c4(270942) c5(270943, 270944, 270945, 270946, 270947, 270948) c6( ) c7(Wb, bM, ac, lR); #c1(SLC24A2) c2(XP_006716813) c3(sodium/potassium/calcium exchanger 2 isoform X2) c4(290489) c5(290490, 290491, 290492, 290493, 290494, 290495) c6( ) c7(Wb, bM, ac, lR); #c1(SLC24A3) c2(NP_065740) c3(sodium/potassium/calcium exchanger 3 precursor) c4(205130) c5(205131, 205132, 205133, 205134, 205135, 205136) c6( ) c7(bM, bgQ, oe, qK, pL, cm, cd); #c1(SLC24A4) c2(NP_705932) c3(sodium/potassium/calcium exchanger 4 isoform 1) c4(246424, 246425) c5(246426, 246427, 246428, 246429, 246430, 246431, 246432, 246433, 246434, 246435, 246436, 246437, 246438) c6( ) c7(bL, cEX, Dy, lR, hk, Y, cz, Ud); #c1(SLC24A4) c2(NP_705933) c3(sodium/potassium/calcium exchanger 4 isoform 2) c4(246439, 246440) c5(246441, 246442, 246443, 246444, 246445, 246446, 246447, 246448, 246449, 246450, 246451, 246452) c6( ) c7(bL, cEX, Dy, lR, hk, Y, cz, Ud); #c1(SLC24A4) c2(NP_705934) c3(sodium/potassium/calcium exchanger 4 isoform 3) c4( ) c5(246453, 246454, 246455, 246456, 246457) c6( ) c7(bL, cEX, Dy, lR, hk, Y, cz, Ud); #c1(SLC24A4) c2(XP_005267399) c3(sodium/potassium/calcium exchanger 4 isoform X6) c4(280966) c5(280967, 280968, 280969, 280970, 280971) c6( ) c7(bL, cEX, Dy, lR, hk, Y, cz, Ud); #c1(SLC24A4) c2(XP_011534738) c3(sodium/potassium/calcium exchanger 4 isoform X1) c4(331201) c5(331202) c6( ) c7(bL, cEX, Dy, lR, hk, Y, cz, Ud); #c1(SLC24A5) c2(NP_995322) c3(sodium/potassium/calcium exchanger 5 precursor) c4(263841, 263842) c5(263843, 263844, 263845, 263846, 263847, 263848, 263849, 263850, 263851, 263852, 263853, 263854) c6( ) c7(W, eR, hk, Y, afQ, aw, ff, cEY, en, aY); #c1(SLC25A1) c2(NP_001243463) c3(tricarboxylate transport protein, mitochondrial isoform b) c4( ) c5(90879) c6( ) c7(T, dA, LX, RX, bB, ci, C, hF, x, PW, agd, ds, iv, iY, bcV, qD, pq, B, cr, gl, aq, Hu, EZ, aot, t, f, lv, bN, ZL, BU, qe, eM, iT, lp, bE, m, k, ER, pw, bt, wS, LT); #c1(SLC25A1) c2(NP_001274316) c3(tricarboxylate transport protein, mitochondrial isoform c) c4(106219) c5(106220, 106221, 106222, 106223, 106224, 106225, 106226, 106227, 106228, 106229) c6( ) c7(T, dA, LX, RX, bB, ci, C, hF, x, PW, agd, ds, iv, iY, bcV, qD, pq, B, cr, gl, aq, Hu, EZ, aot, t, f, lv, bN, ZL, BU, qe, eM, iT, lp, bE, m, k, ER, pw, bt, wS, LT); #c1(SLC25A1) c2(NP_005975) c3(tricarboxylate transport protein, mitochondrial isoform a precursor) c4(160923) c5(160924, 160925, 160926, 160927, 160928, 160929, 160930) c6( ) c7(T, dA, LX, RX, bB, ci, C, hF, x, PW, agd, ds, iv, iY, bcV, qD, pq, B, cr, gl, aq, Hu, EZ, aot, t, f, lv, bN, ZL, BU, qe, eM, iT, lp, bE, m, k, ER, pw, bt, wS, LT); #c1(SLC25A10) c2(NP_001257817) c3(mitochondrial dicarboxylate carrier isoform 1) c4(95664) c5(95665, 95666, 95667, 95668, 95669, 95670) c6( ) c7(zQ, ag, hl, ci, aWY, jj, zP, blm); #c1(SLC25A10) c2(NP_001257882) c3(mitochondrial dicarboxylate carrier isoform 3) c4( ) c5(95736) c6( ) c7(zQ, ag, hl, ci, aWY, jj, zP, blm); #c1(SLC25A10) c2(NP_036272) c3(mitochondrial dicarboxylate carrier isoform 2) c4(172768, 172769) c5(172770, 172771, 172772, 172773, 172774, 172775, 172776, 172777, 172778, 172779, 172780, 172781) c6( ) c7(zQ, ag, hl, ci, aWY, jj, zP, blm); #c1(SLC25A10) c2(XP_005257131) c3(mitochondrial dicarboxylate carrier isoform X1) c4(274704) c5(274705, 274706, 274707, 274708, 274709) c6( ) c7(zQ, ag, hl, ci, aWY, jj, zP, blm); #c1(SLC25A12) c2(NP_003696) c3(calcium-binding mitochondrial carrier protein Aralar1) c4(140226, 140227) c5(140228, 140229, 140230, 140231, 140232, 140233) c6( ) c7(rz, cEZ, By, cbE, ais, y, bN); #c1(SLC25A12) c2(XP_011510372) c3(calcium-binding mitochondrial carrier protein Aralar1 isoform X3) c4( ) c5(300907, 300908, 300909, 300910, 300911, 300912) c6( ) c7(rz, cEZ, By, cbE, ais, y, bN); #c1(SLC25A13) c2(NP_001153682) c3(calcium-binding mitochondrial carrier protein Aralar2 isoform 1) c4(60848) c5(60849, 60850, 60851, 60852, 60853, 60854) c6( ) c7(cFb, Na, bEN, fc, hl, cFa, bEM, Nc, byM, cP, ais, hJ, jw, B, bN); #c1(SLC25A13) c2(NP_055066) c3(calcium-binding mitochondrial carrier protein Aralar2 isoform 2) c4(178692, 178693) c5(178694, 178695, 178696, 178697, 178698, 178699) c6( ) c7(cFb, Na, bEN, fc, hl, cFa, bEM, Nc, byM, cP, ais, hJ, jw, B, bN); #c1(SLC25A13) c2(XP_006715894) c3(calcium-binding mitochondrial carrier protein Aralar2 isoform X1) c4( ) c5(289886, 289887, 289888, 289889, 289890, 289891) c6( ) c7(cFb, Na, bEN, fc, hl, cFa, bEM, Nc, byM, cP, ais, hJ, jw, B, bN); #c1(SLC25A14) c2(NP_001269124) c3(brain mitochondrial carrier protein 1 isoform 1 precursor) c4(100495, 100496, 100497) c5(100498, 100499, 100500, 100501, 100502, 100503, 100504, 100505, 100506, 100507, 100508, 100509, 100510, 100511, 100512, 100513, 100514, 100515) c6( ) c7(v); #c1(SLC25A14) c2(NP_001269125) c3(brain mitochondrial carrier protein 1 isoform 2 precursor) c4(100516) c5(100517, 100518, 100519, 100520, 100521, 100522, 100523) c6( ) c7(v); #c1(SLC25A14) c2(NP_001269126) c3(brain mitochondrial carrier protein 1 isoform 3 precursor) c4(100524) c5(100525, 100526, 100527, 100528, 100529, 100530) c6( ) c7(v); #c1(SLC25A14) c2(NP_001269127) c3(brain mitochondrial carrier protein 1 isoform 4) c4( ) c5(100531, 100532, 100533, 100534, 100535) c6( ) c7(v); #c1(SLC25A14) c2(XP_011529704) c3(brain mitochondrial carrier protein 1 isoform X1) c4(325334) c5( ) c6( ) c7(v); #c1(SLC25A15) c2(NP_055067) c3(mitochondrial ornithine transporter 1) c4(178700, 178701, 178702) c5(178703, 178704, 178705, 178706, 178707, 178708, 178709, 178710, 178711, 178712, 178713, 178714, 178715, 178716, 178717, 178718, 178719, 178720, 178721, 178722, 178723, 178724, 178725, 178726, 178727, 178728) c6( ) c7(ahW, bDw, aip, cmt, jA, ali, QK, avx); #c1(SLC25A16) c2(XP_006718050) c3(graves disease carrier protein isoform X2) c4(291476) c5(291477, 291478, 291479, 291480, 291481) c6( ) c7(ck, xl, T, kh, sS, d, c, nO, Jl, aW, aT, aA, fd, aF, fb, bt, jf); #c1(SLC25A16) c2(XP_011538514) c3(graves disease carrier protein isoform X1) c4(336263) c5(336264, 336265, 336266, 336267, 336268, 336269, 336270) c6( ) c7(ck, xQ, T, kh, sS, d, c, nD, Jl, aW, aT, aA, fd, aF, fb, bt, jf); #c1(SLC25A18) c2(NP_001290413) c3(mitochondrial glutamate carrier 2) c4(115380) c5(115381, 115382, 115383, 115384, 115385, 115386, 115387, 115388) c6( ) c7(ae, fr); #c1(SLC25A18) c2(XP_011544451) c3(mitochondrial glutamate carrier 2 isoform X2) c4( ) c5(343604, 343605, 343606) c6( ) c7(ae, fr); #c1(SLC25A19) c2(XP_006722070) c3(mitochondrial thiamine pyrophosphate carrier isoform X1) c4(294568, 294569) c5(294570, 294571, 294572, 294573, 294574, 294575, 294576, 294577, 294578, 294579, 294580, 294581, 294582, 294583, 294584, 294585, 294586, 294587, 294588, 294589, 294590, 294591, 294592, 294593, 294594, 294595, 294596, 294597) c6( ) c7(d, ob, cFc, cm, fu, QK, cFd); #c1(SLC25A2) c2(NP_114153) c3(mitochondrial ornithine transporter 2) c4(222383) c5(222384, 222385, 222386, 222387, 222388, 222389) c6( ) c7(ahW, bDw); #c1(SLC25A20) c2(NP_000378) c3(mitochondrial carnitine/acylcarnitine carrier protein) c4(8302, 8303) c5(8304, 8305, 8306, 8307, 8308, 8309, 8310, 8311, 8312, 8313, 8314, 8315) c6( ) c7(T, id, e, aDE, bGr, cR, aj, v, j, J, qH, dL, bK, K, eL, af, dx, jf, xQ, iF, A, s, cW, ajx, ge, cl, aNJ, pg, cS, pw, be, sZ, B, jr); #c1(SLC25A21) c2(NP_001164641) c3(mitochondrial 2-oxodicarboxylate carrier isoform 2) c4(67121) c5(67122, 67123, 67124, 67125, 67126, 67127) c6( ) c7(bM, T, ha, fd, bB, n, gA, bS, ct, ci, q, zo, ad, ip, aT, h, dz, hn, K, ae, et, aW, Y, r, aq, Z, cm, iR, dR, ck, bF, bE, MC, B); #c1(SLC25A21) c2(NP_085134) c3(mitochondrial 2-oxodicarboxylate carrier isoform 1) c4(219388) c5(219389, 219390, 219391, 219392, 219393, 219394) c6( ) c7(bM, T, ha, fd, bB, n, gA, bS, ct, ci, q, zo, ad, ip, aT, h, dz, hn, K, ae, et, aW, Y, r, aq, Z, cm, iR, dR, ck, bF, bE, MC, B); #c1(SLC25A22) c2(XP_011518673) c3(mitochondrial glutamate carrier 1 isoform X1) c4(312992, 312993, 312994, 312995, 312996, 312997, 312998, 312999, 313000, 313001, 313002) c5(313003, 313004, 313005, 313006, 313007, 313008, 313009, 313010, 313011, 313012, 313013, 313014, 313015, 313016, 313017, 313018, 313019, 313020, 313021, 313022, 313023, 313024, 313025, 313026, 313027, 313028, 313029, 313030, 313031, 313032, 313033, 313034, 313035, 313036, 313037, 313038, 313039, 313040, 313041, 313042, 313043, 313044, 313045, 313046, 313047, 313048, 313049, 313050, 313051, 313052, 313053, 313054, 313055, 313056, 313057, 313058, 313059, 313060, 313061, 313062, 313063, 313064, 313065, 313066, 313067, 313068, 313069, 313070, 313071, 313072, 313073, 313074, 313075, 313076) c6( ) c7(crP, yr, ha, lm); #c1(SLC25A23) c2(NP_077008) c3(calcium-binding mitochondrial carrier protein SCaMC-3) c4(214715, 214716) c5(214717, 214718, 214719, 214720, 214721, 214722, 214723, 214724, 214725, 214726, 214727, 214728) c6( ) c7(bF, T, K, m, g, t, iu); #c1(SLC25A23) c2(XP_011526579) c3(calcium-binding mitochondrial carrier protein SCaMC-3 isoform X4) c4( ) c5(320874) c6( ) c7(bF, T, K, m, g, t, iu); #c1(SLC25A23) c2(XP_011526585) c3(calcium-binding mitochondrial carrier protein SCaMC-3 isoform X10) c4(320875) c5(320876, 320877, 320878, 320879, 320880) c6( ) c7(bF, T, K, m, g, t, iu); #c1(SLC25A23) c2(XP_011526586) c3(calcium-binding mitochondrial carrier protein SCaMC-3 isoform X11) c4(320881) c5(320882, 320883, 320884, 320885, 320886, 320887) c6( ) c7(bF, T, K, m, g, t, iu); #c1(SLC25A23) c2(XP_011526587) c3(calcium-binding mitochondrial carrier protein SCaMC-3 isoform X12) c4(320888) c5(320889, 320890, 320891, 320892, 320893, 320894) c6( ) c7(bF, T, K, m, g, t, iu); #c1(SLC25A25) c2(NP_001006642) c3(calcium-binding mitochondrial carrier protein SCaMC-2 isoform b) c4(22317) c5(22318, 22319, 22320, 22321, 22322, 22323) c6( ) c7(Na, Y, Nc); #c1(SLC25A25) c2(NP_001006643) c3(calcium-binding mitochondrial carrier protein SCaMC-2 isoform c precursor) c4(22324) c5(22325, 22326, 22327, 22328, 22329, 22330) c6( ) c7(Na, Y, Nc); #c1(SLC25A25) c2(NP_001252543) c3(calcium-binding mitochondrial carrier protein SCaMC-2 isoform 5 precursor) c4(94238) c5(94239, 94240, 94241, 94242, 94243, 94244) c6( ) c7(Na, Y, Nc); #c1(SLC25A25) c2(NP_443133) c3(calcium-binding mitochondrial carrier protein SCaMC-2 isoform a) c4(229589, 229590) c5(229591, 229592, 229593, 229594, 229595, 229596, 229597) c6( ) c7(Na, Y, Nc); #c1(SLC25A25) c2(XP_005251745) c3(calcium-binding mitochondrial carrier protein SCaMC-2 isoform X1) c4(271138) c5(271139, 271140, 271141, 271142, 271143, 271144) c6( ) c7(Na, Y, Nc); #c1(SLC25A25) c2(XP_005251746) c3(calcium-binding mitochondrial carrier protein SCaMC-2 isoform X4) c4( ) c5(271145, 271146, 271147, 271148, 271149) c6( ) c7(Na, Y, Nc); #c1(SLC25A27) c2(NP_001190980) c3(mitochondrial uncoupling protein 4 isoform 2) c4( ) c5(80366) c6( ) c7(bL, cm, V, aM, uu); #c1(SLC25A27) c2(NP_001190981) c3(mitochondrial uncoupling protein 4 isoform 3) c4(80367) c5(80368, 80369, 80370, 80371, 80372, 80373, 80374, 80375, 80376, 80377, 80378, 80379) c6( ) c7(bL, cm, V, aM, uu); #c1(SLC25A27) c2(NP_004268) c3(mitochondrial uncoupling protein 4 isoform 1) c4(144721, 144722) c5(144723, 144724, 144725, 144726, 144727, 144728, 144729, 144730, 144731, 144732, 144733, 144734) c6( ) c7(bL, cm, V, aM, uu); #c1(SLC25A27) c2(XP_005249540) c3(mitochondrial uncoupling protein 4 isoform X1) c4(269542, 269543, 269544, 269545) c5( ) c6( ) c7(bL, cm, V, aM, uu); #c1(SLC25A27) c2(XP_005249542) c3(mitochondrial uncoupling protein 4 isoform X2) c4( ) c5(269546, 269547, 269548, 269549, 269550, 269551, 269552, 269553, 269554, 269555, 269556, 269557, 269558, 269559, 269560, 269561, 269562) c6( ) c7(bL, cm, V, aM, uu); #c1(SLC25A3) c2(NP_002626) c3(phosphate carrier protein, mitochondrial isoform b precursor) c4(130419) c5(130420, 130421, 130422, 130423, 130424, 130425, 130426, 130427, 130428, 130429, 130430, 130431, 130432, 130433, 130434, 130435, 130436, 130437) c6( ) c7(bL, T, yS, aA, cz, aj, dG, v, yO, fr, V, eA, J, ad, av, bza, aT, h, vc, Ou, ey, K, cFe, ae, A, bug, iU, aW, Y, eH, af, as, pm, eF, t, bz, dr, ck, es, SE, eo, m); #c1(SLC25A3) c2(NP_005879) c3(phosphate carrier protein, mitochondrial isoform a precursor) c4(160081) c5(160082, 160083, 160084, 160085, 160086, 160087, 160088, 160089, 160090, 160091) c6( ) c7(bL, T, yS, aA, cz, aj, dG, v, yO, fr, V, eA, J, ad, av, bza, aT, h, vc, Ou, ey, K, cFe, ae, A, bug, iU, aW, Y, eH, aF, as, pm, eF, t, bz, dr, ck, es, SE, eo, m); #c1(SLC25A36) c2(NP_001098117) c3(solute carrier family 25 member 36 isoform a) c4(41343) c5(41344, 41345, 41346, 41347, 41348, 41349) c6( ) c7(V, aE, ci); #c1(SLC25A36) c2(NP_060625) c3(solute carrier family 25 member 36 isoform b) c4(196891) c5(196892, 196893, 196894, 196895, 196896, 196897) c6( ) c7(V, aE, ci); #c1(SLC25A37) c2(NP_057696) c3(mitoferrin-1) c4(192391) c5(192392, 192393, 192394, 192395, 192396, 192397) c6( ) c7(lD, T, am, dA, hV, fG, aw, fN, aA, bB, n, aj, bS, v, wV, fd, PJ, aY, V, q, amW, J, eA, ip, x, eW, py, aE, fm, M, ZQ, aT, cy, oW, bl, K, zp, ji, bPQ, N, bA, ly, A, d, aW, Y, oV, xs, hl, r, fe, af, zC, nr, t, lx, ek, ck, es, lt, ac, c, wg, dB, wO, bE, iA, cj, uJ, m, k, fD, lC, aNl, B); #c1(SLC25A38) c2(NP_060345) c3(solute carrier family 25 member 38) c4(195522) c5(195523, 195524, 195525, 195526, 195527, 195528) c6( ) c7(bDt, U, ac, kA); #c1(SLC25A4) c2(NP_001142) c3(ADP/ATP translocase 1) c4(60077) c5(60078, 60079, 60080, 60081, 60082, 60083) c6( ) c7(vq, bM, T, AY, gU, eR, aFc, bO, mS, aNG, sl, pM, bgR, ad, dP, cc, Bc, mW, oj, aT, vH, h, LE, bK, K, dL, mY, mV, cFf, ae, af, awL, cFg, eL, mU, B, d, ND, dH, aW, xs, t, kC, bz, mZ, ek, mX, Qi, akQ, ac, ctb, cA, uw, mx, at, rx, m, mT, Al); #c1(SLC25A40) c2(NP_061331) c3(solute carrier family 25 member 40) c4(200052) c5(200053, 200054, 200055, 200056, 200057, 200058) c6( ) c7(ff, en); #c1(SLC25A41) c2(NP_775908) c3(solute carrier family 25 member 41) c4(250663, 250664) c5(250665, 250666, 250667, 250668, 250669, 250670, 250671, 250672, 250673, 250674, 250675) c6( ) c7(aW, aT); #c1(SLC25A41) c2(XP_011526228) c3(solute carrier family 25 member 41 isoform X1) c4( ) c5(320491) c6( ) c7(aW, aT); #c1(SLC25A42) c2(NP_848621) c3(mitochondrial coenzyme A transporter SLC25A42) c4(254008) c5(254009, 254010, 254011, 254012, 254013, 254014) c6( ) c7(MD); #c1(SLC25A43) c2(NP_660348) c3(solute carrier family 25 member 43) c4(241021, 241022, 241023) c5(241024, 241025, 241026, 241027, 241028, 241029, 241030, 241031, 241032, 241033, 241034, 241035, 241036, 241037, 241038, 241039, 241040, 241041) c6( ) c7(aW, T, bi, bdM, aT); #c1(SLC25A45) c2(NP_001265179) c3(solute carrier family 25 member 45 isoform c) c4(97920) c5(97921, 97922, 97923, 97924, 97925, 97926) c6( ) c7(Y); #c1(SLC25A45) c2(NP_001287749) c3(solute carrier family 25 member 45 isoform d) c4(112788) c5(112789, 112790, 112791, (12792, 112793, 112794) c6( ) c7(Y); #c1(SLC25A45) c2(XP_011543244) c3(solute carrier family 25 member 45 isoform X2) c4(342028) c5(342029, 342030, 342031, 342032, 342033, 342034, 342035, 342036, 342037, 342038, 342039, 342040) c6( ) c7(Y); #c1(SLC25A45) c2(XP_011543250) c3(solute carrier family 25 member 45 isoform X4) c4(342041) c5(342042, 342043, 342044, 342045, 342046, 342047) c6( ) c7(Y); #c1(SLC25A45) c2(XP_011543251) c3(solute carrier family 25 member 45 isoform X1) c4(342048) c5(342049, 342050, 342051, 342052, 342053, 342054, 342055, 342056) c6( ) c7(Y); #c1(SLC25A46) c2(NP_001290178) c3(solute carrier family 25 member 46 isoform 2) c4(115062) c5(115063, 115064, 115065, 115066, 115067, 115068) c6( ) c7(bM, e); #c1(SLC25A46) c2(NP_001290179) c3(solute carrier family 25 member 46 isoform 3) c4(115069, 115070, 115071, 115072) c5(115073, 115074, 115075, 115076, 115077, 115078, 115079, 115080, 115081, 115082, 115083, 115084, 115085, 115086, 115087, 115088, 115089, 115090, 115091, 115092, 115093, 115094, 115095, 115096, 115097, 115098, 115099) c6( ) c7(bM, e); #c1(SLC25A46) c2(NP_620128) c3(solute carrier family 25 member 46 isoform 1) c4(237315) c5(237316, 237317, 237318, 237319, 237320, 237321, 237322) c6( ) c7(bM, e); #c1(SLC25A47) c2(NP_997000) c3(solute carrier family 25 member 47) c4(264923, 264924) c5(264925, 264926, 264927, 264928, 264929, 264930, 264931) c6( ) c7(B); #c1(SLC25A5) c2(NP_001143) c3(ADP/ATP translocase 2) c4(60084) c5(60085, 60086, 60087, 60088, 60089, 60090) c6( ) c7(T, DM, Uc, eR, aNG, ci, nA, ad, cc, kH, aT, K, ae, je, aW, hl, aq, g, pv, es, iM, mx, hr, rk, gT, B); #c1(SLC25A52) c2(NP_001029344) c3(solute carrier family 25 member 52) c4(30788, 30789) c5(30790, 30791, 30792, 30793, 30794, 30795, 30796, 30797, 30798, 30799, 30800, 30801, 30802, 30803, 30804) c6( ) c7(hb, aW, aT); #c1(SLC25A6) c2(NP_001627) c3(ADP/ATP translocase 3) c4(119338, 119339) c5(119340, 119341, 119342, 119343, 119344, 119345, 119346, 119347, 119348, 119349, 119350, 119351) c6( ) c7(d, mx, aFc, bO, h, bK, K, kC); #c1(SLC26A1) c2(NP_071325) c3(sulfate anion transporter 1 isoform a) c4(210983) c5(210984, 210985, 210986, 210987, 210988, 210989, 210990, 210991, 210992, 210993) c6( ) c7(hq, vK, Tl, s, Y, MB, bO, aT, fd, bS, bK, eL, cd, le); #c1(SLC26A1) c2(NP_602297) c3(sulfate anion transporter 1 isoform b) c4(235659) c5(235660, 235661, 235662, 235663, 235664, 235665) c6( ) c7(hq, vK, Tl, s, Y, MB, b, aT, fd, bS, bK, eL, cd, Qe); #c1(SLC26A2) c2(NP_000103) c3(sulfate transporter) c4(1787, 1788) c5(1789, 1790, 1791, 1792, 1793, 1794, 1795, 1796, 1797, 1798, 1799, 1800) c6( ) c7(T, aA, bB, cL, ade, o, XW, av, ib, ry, aT, alj, cFh, rv, bA, eG, s, cFk, Az, dq, aF, bQ, dr, vk, cfj, eM, bE, cFi, ln); #c1(SLC26A3) c2(XP_011514169) c3(chloride anion exchanger isoform X1) c4(306768, 306769, 306770) c5(306771, 306772, 306773, 306774, 306775, 306776, 306777, 306778, 306779, 306780, 306781, 306782, 306783, 306784, 306785, 306786, 306787, 306788) c6( ) c7(T, al, jx, jh, bB, aj, cFl, V, q, J, hX, AW, fX, Tl, lv, ry, lz, ju, hD, bzw, KV, d, s, apn, r, akB, dq, aMF, g, hf, iV, dr, gk, vK, bE, Vu, aer, aZL, ln, U, cj, X, eo, B, ao); #c1(SLC26A4) c2(NP_000432) c3(pendrin) c4(8916, 8917, 8918, 8919, 8920) c5(8921, 8922, 8923, 8924, 8925, 8926, 8927, 8928, 8929, 8930, 8931, 8932, 8933, 8934, 8935, 8936, 8937, 8938, 8939, 8940, 8941, 8942, 8943, 8944) c6( ) c7(bDV, bM, T, bCB, akd, fR, aTk, bCy, bzb, cz, hz, auq, j, ci, ad, Vp, b, hd, xy, RN, nX, ry, aMT, aT, hD, h, K, on, yF, bCA, s, aW, lo, bwF, ale, dr, DF, bCJ, ck, cT, vK, wg, nB, auz, mG, ln, at, Hy, i, gT, bt, Bh); #c1(SLC26A4) c2(XP_006716088) c3(pendrin isoform X2) c4( ) c5(290030, 290031, 290032, 290033, 290034, 290035) c6( ) c7(bDV, bM, T, bCB, akd, fR, aTk, bCy, bzb, cz, hz, auq, j, ci, ad, Vp, b, hd, xy, RN, nX, ry, aMT, aT, hD, h, K, on, yF, bCA, s, aW, lo, bwF, ale, dr, DF, bCJ, ck, cT, vK, wg, nB, auz, mG, ln, at, Hy, i, gT, bt, Bh); #c1(SLC26A5) c2(NP_001161434) c3(prestin isoform e) c4(65791, 65792, 65793, 65794) c5(65795, 65796, 65797, 65798, 65799, 65800, 65801, 65802, 65803, 65804, 65805, 65806, 65807, 65808, 65809, 65810, 65811, 65812, 65813, 65814, 65815, 65816, 65817, 65818, 65819, 65820, 65821, 65822, 65823, 65824, 65825, 65826, 65827) c6( ) c7(Kd, vK, ry, ln, gT, cFm, vi); #c1(SLC26A5) c2(NP_996766) c3(prestin isoform b) c4(264487) c5(264488, 264489, 264490, 264491, 264492, 264493) c6( ) c7(Kd, vK, ry, ln, gT, cFm, vi); #c1(SLC26A5) c2(NP_996767) c3(prestin isoform c) c4(264494) c5(264495, 264496, 264497, 264498, 264499, 264500) c6( ) c7(Kd, vK, ry, ln, gT, cFm, vi); #c1(SLC26A5) c2(NP_996768) c3(prestin isoform d) c4(264501) c5(264502, 264503, 264504, 264505, 264506, 264507) c6( ) c7(Kd, vK, ry, ln, gT, cFm, vi); #c1(SLC26A5) c2(XP_011514472) c3(prestin isoform X1) c4(307166, 307167, 307168, 307169) c5(307170, 307171, 307172, 307173, 307174, 307175, 307176, 307177, 307178, 307179, 307180, 307181, 307182, 307183, 307184, 307185, 307186, 307187, 307188) c6( ) c7(Kd, vK, ry, ln, gT, cFm, vi); #c1(SLC26A6) c2(NP_001035544) c3(solute carrier family 26 member 6 isoform 4) c4( ) c5(32911, 32912, 32913, 32914, 32915) c6( ) c7(vK, VJ, s, bpN, Fm, MB, cz, Hl, aAN); #c1(SLC26A6) c2(NP_001268661) c3(solute carrier family 26 member 6 isoform 5) c4(100092) c5(100093, 100094, 100095, 100096, 100097, 100098) c6( ) c7(vK, VJ, s, bpN, Fm, MB, cz, Hl, aAN); #c1(SLC26A6) c2(NP_001268662) c3(solute carrier family 26 member 6 isoform 6) c4(100099) c5(100100, 100101, 100102, 100103, 100104, 100105) c6( ) c7(vK, VJ, s, bpN, Fm, MB, cz, Hl, aAN); #c1(SLC26A6) c2(NP_075062) c3(solute carrier family 26 member 6 isoform 1) c4(213623, 213624) c5(213625, 213626, 213627, 213628, 213629, 213630, 213631) c6( ) c7(vK, VJ, s, bpN, Fm, MB, cz, Hl, aAN); #c1(SLC26A6) c2(NP_599025) c3(solute carrier family 26 member 6 isoform 2) c4(235593) c5(235594, 235595, 235596, 235597, 235598, 235599) c6( ) c7(vK, VJ, s, bpN, Fm, MB, cz, Hl, aAN); #c1(SLC26A6) c2(NP_602298) c3(solute carrier family 26 member 6 isoform 3) c4(235666, 235667) c5(235668, 235669, 235670, 235671, 235672, 235673, 235674, 235675, 235676, 235677, 235678, 235679) c6( ) c7(vK, VJ, s, bpN, Fm, MB, cz, Hl, aAN); #c1(SLC26A7) c2(NP_439897) c3(anion exchange transporter isoform a) c4(229170) c5(229171, 229172, 229173, 229174, 229175, 229176, 229177, 229178, 229179, 229180, 229181, 229182, 229183, 229184, 229185, 229186) c6( ) c7(Hx, vK, VJ, s, Fm, bBQ); #c1(SLC26A7) c2(NP_599028) c3(anion exchange transporter isoform b) c4(235607, 235608) c5(235609, 235610, 235611, 235612, 235613, 235614) c6( ) c7(Hx, vK, VJ, s, Fm, BBQ); #c1(SLC26A8) c2(NP_001180405) c3(testis anion transporter 1 isoform a) c4(73954) c5(73955, 73956, 73957, 73958, 73959, 73960, 73961, 73962, 73963, 73964) c6( ) c7(gU, ac, VJ, eR, eL, Fm, e, eu, aw, h, dL, j, clD, qN, pt, ae, aR, ad); #c1(SLC26A8) c2(NP_619732) c3(testis anion transporter 1 isoform b) c4(237196) c5(237197, 237198, 237199, 237200, 237201, 237202) c6( ) c7(gU, ac, VJ, eR, eL, Fm, e, eu, aw, h, dL, j, clD, qN, pt, ae, aR, ad); #c1(SLC26A9) c2(NP_599152) c3(solute carrier family 26 member 9 isoform b) c4(235641, 235642) c5(235643, 235644, 235645, 235646, 235647, 235648, 235649, 235650, 235651, 235652, 235653, 235654, 235655, 235656, 235657, 235658) c6( ) c7(bL, bM, DF, cFp, jx, aMp, aj, cFn, J, cFo, aa); #c1(SLC26A9) c2(XP_011507425) c3(solute carrier family 26 member 9 isoform X3) c4( ) c5(297349, 297350, 297351) c6( ) c7(bL, bM, DF, cFp, jx, aMp, aj, cFn, J, cFo, aa); #c1(SLC27A1) c2(XP_011526304) c3(long-chain fatty acid transport protein 1 isoform X2) c4(320587) c5(320588, 320589, 320590, 320591, 320592, 320593) c6( ) c7(bL, bxy, e, aw, jy, mf, v, ei, dQ, nS); #c1(SLC27A1) c2(XP_011526305) c3(long-chain fatty acid transport protein 1 isoform X1) c4(320594) c5(320595, 320596, 320597, 320598, 320599, 320600, 320601, 320602, 320603, 320604) c6( ) c7(bL, bxy, e, aw, jy, mf, v, ei, dQ, nS); #c1(SLC27A2) c2(NP_001153101) c3(very long-chain acyl-CoA synthetase isoform 2) c4(60428) c5(60429, 60430, 60431, 60432, 60433, 60434, 60435) c6( ) c7(mD, aW, dF, aT, eA, bz, af); #c1(SLC27A2) c2(NP_003636) c3(very long-chain acyl-CoA synthetase isoform 1) c4(139790) c5(139791, 139792, 139793, 139794, 139795, 139796) c6( ) c7(mD, aW, dF, aT, eA, bz, af); #c1(SLC27A3) c2(NP_077306) c3(long-chain fatty acid transport protein 3) c4(215151, 215152) c5(215153, 215154, 215155, 215156, 215157, 215158, 215159, 215160, 215161, 215162, 215163, 215164) c6( ) c7(T, d, dB, Gp, bz, eA); #c1(SLC27A4) c2(NP_005085) c3(long-chain fatty acid transport protein 4) c4(152751, 152752) c5(152753, 152754, 152755, 152756, 152757, 152758, 152759, 152760, 152761, 152762, 152763, 152764) c6( ) c7(bL, cFq, vj, fc, aA, fn, lo, v, aF, ei, nS, ako, bxJ); #c1(SLC27A5) c2(NP_036386) c3(bile acyl-CoA synthetase precursor) c4(173467) c5(173468, 173469, 173470, 173471, 173472, 173473) c6( ) c7(oO, bL, T, bM, fM, fG, df, hF, bW, h, vB, K, zy, MG, qE, uQ, aky, aKD, fc, hl, aq, anW, pb, dr, LB, ig, dc, do, uR, at, Br, dT, fD, ei, B); #c1(SLC28A1) c2(NP_004204) c3(sodium/nucleoside cotransporter 1 isoform 1) c4(144310) c5(144311, 144312, 144313, 144314, 144315, 144316, 144317, 144318, 144319, 144320) c6( ) c7(gV, aC, av, eJ, af, hG, aE, aW, eM, cj, aT, GR, h, bX, g, bt, Cr, B); #c1(SLC28A1) c2(NP_964014) c3(sodium/nucleoside cotransporter 1 isoform 2) c4(262629) c5(262630, 262631, 262632, 262633, 262634, 262635) c6( ) c7(gV, aC, av, eJ, af, hG, aE, aW, eM, cj, aT, GR, h, bX, g, bt, Cr, B); #c1(SLC28A1) c2(XP_011520509) c3(sodium/nucleoside cotransporter 1 isoform X2) c4(315422) c5( ) c6( ) c7(gV, aC, av, eJ, af, hG, aE, aW, eM, cj, al, GR, h, bX, g, bt, Cr, B); #c1(SLC28A1) c2(XP_011520512) c3(sodium/nucleoside cotransporter 1 isoform X5) c4(315423) c5(315424, 315425, 315426, 315427, 315428, 315429) c6( ) c7(gV, aC, av, eJ, af, hG, aE, aW, eM, cj, aT, GR, h, bX, g, bt, Cr, B); #c1(SLC28A1) c2(XP_011520515) c3(sodium/nucleoside cotransporter 1 isoform X8) c4(315430) c5(315431, 315432, 315433, 315434, 315435, 315436) c6( ) c7(gV, aC, av, eJ, af, hG, aE, aW, eM, cj, aT, GR, h, bX, g, bt, Cr, B); #c1(SLC28A1) c2(XP_011520519) c3(sodium/nucleoside cotransporter 1 isoform X1) c4(315437) c5(315438, 315439, 315440, 315441, 315442, 315443) c6( ) c7(gV, aC, av, eJ, af, hG, aE, aW, eM, cj, al, GR, h, bX, g, bt, Cr, B); #c1(SLC28A1) c2(XP_011520520) c3(sodium/nucleoside cotransporter 1 isoform X11) c4( ) c5(315444, 315445, 315446, 315447, 315448, 315449) c6( ) c7(gV, aC, av, eJ, af, hG, aE, aW, eM, cj, aT, GR, h, bX, g, bt, Cr, B); #c1(SLC28A2) c2(XP_011520500) c3(sodium/nucleoside cotransporter 2 isoform X1) c4(315415) c5(315416, 315417, 315418, 315419, 315420, 315421) c6( ) c7(oW, cj, dy, bq, Cr, GR, vi); #c1(SLC28A3) c2(NP_001186562) c3(solute carrier family 28 member 3) c4(77821, 77822) c5(77823, 77824, 77825, 77826, 77827, 77828, 77829, 77830, 77831, 77832, 77833, 77834) c6( ) c7(gV, hG, jx, oW, cj, h, bq, GR); #c1(SLC29A1) c2(NP_001071643) c3(equilibrative nucleoside transporter 1 isoform 1) c4( ) c5(35382, 35383, 35384, 35385, 35386, 35387, 35388, 35389, 35390, 35391, 35392, 35393, 35394, 35395, 35396, 35397, 35398, 35399, 35400, 35401, 35402, 35403, 35404, 35405, 35406, 35407, 35408, 35409, 35410, 35411, 35412, 35413, 35414, 35415, 35416) c6( ) c7(aC, jx, eX, lu, eJ, x, av, F, aE, oW, h, eV, bq, Cr, eu, je, GR, aap, iM, eM, mg, cj, k, fD, bt); #c1(SLC29A1) c2(NP_001291391) c3(equilibrative nucleoside transporter 1 isoform 5) c4(115859) c5(115860, 115861, 115862, 115863, 115864, 115865) c6( ) c7(aC, jx, eX, lu, BJ, x, av, F, aE, oW, h, eV, bq, Cr, eu, je, GR, aap, iM, eM, mg, cj, k, fD, bt); #c1(SLC29A1) c2(NP_001291392) c3(equilibrative nucleoside transporter 1 isoform 2) c4(115866) c5(115867, 115868, 115869) c6( ) c7(aC, jx, eX, lu, eJ, x, av, F, aE, oW, h, eV, bq, Cr, eu, je, GR, aap, iM, BM, mg, cj, k, fD, bt); #c1(SLC29A1) c2(NP_001291394) c3(equilibrative nucleoside transporter 1 isoform 3) c4(115876) c5(115877, 115878, 115879, 115880) c6( ) c7(aC, jx, eX, lu, eJ, x, av, F, aE, oW, h, eV, bq, Cr, eu, je, GR, aap, iM, eM, mg, cj, k, fD, bt); #c1(SLC29A1) c2(NP_001291395) c3(equilibrative nucleoside transporter 1 isoform 4) c4( ) c5(115881, 115882) c6( ) c7(aC, jx, eX, lu, eJ, x, av, F, aE, oW, h, eV, bq, Cr, eu, je, GR, aap, iM, eM, mg, cj, k, fD, bt); #c1(SLC29A2) c2(XP_011543277) c3(equilibrative nucleoside transporter 2 isoform X2) c4(342071) c5(342072, 342073, 342074, 342075, 342076, 342077, 342078, 342079, 342080, 342081, 342082, 342083, 342084, 342085) c6( ) c7(T, pY, jx, dG, hF, x, Cr, af, bVG, Nr, Y, po, bw, GR, Nq, ck, EW, hL, cj, Fk, k, cFr, fD, bt, B); #c1(SLC29A3) c2(NP_060814) c3(equilibrative nucleoside transporter 3 isoform a) c4(197961) c5(197962, 197963, 197964, 197965, 197966, 197967) c6( ) c7(bGG, T, A, cl, aE, bD, cCL, aj, bS, Lq, ct, lQ, mv, Z, aF, Cr, cFs, J, UE); #c1(SLC29A4) c2(XP_006715730) c3(equilibrative nucleoside transporter 4 isoform X1) c4(289740, 289741) c5(289742, 289743, 289744, 289745, 289746, 289747, 289748, 289749, 289750, 289751, 289752, 289753, 289754, 289755, 289756) c6( ) c7(cy, Cr, aF); #c1(SLC2A10) c2(NP_110404) c3(solute carrier family 2, facilitated glucose transporter member 10) c4(220102) c5(220103, 220104, 220105, 220106, 220107, 220108) c6( ) c7(bL, bM, kh, Av, aj, Ea, rc, J, bW, nd, aT, aSX, bf, aW, nf, g, bz, aSZ, LB, Bv, agl, iu); #c1(SLC2A11) c2(NP_001020109) c3(solute carrier family 2, facilitated glucose transporter member 11 isoform c) c4(28179, 28180) c5(28181, 28182, 28183, 28184, 28185, 28186) c6( ) c7(bL, bf, Av, fd, ae, ad); #c1(SLC2A11) c2(NP_001020110) c3(solute carrier family 2, facilitated glucose transporter member 11 isoform b) c4(28187) c5(28188, 28189, 28190, 28191, 28192, 28193, 28194, 28195, 28196, 28197, 28198, 28199) c6( ) c7(bL, bf, Av, fd, ae, ad); #c1(SLC2A1) c2(NP_001269793) c3 (solute carrier family 2, facilitated glucose transporter member 11 isoform d) c4(102837, 102838) c5(102839, 102840, 102841, 102842, 102843, 102844, 102845, 102846, 102847, 102848, 102849, 102850) c6( ) c7(bL, bf, Av, fd, ae, ad); #c1(SLC2A1l) c2(NP_110434) c3(solute carrier family 2, facilitated glucose transporter member 11 isoform a) c4(220347) c5(220348, 220349, 220350, 220351, 220352, 220353) c6( ) c7(bL, bf, Av, fd, ae, ad); #c1(SLC2A12) c2(NP_660159) c3(solute carrier family 2, facilitated glucose transporter member (2) c4(240454) c5(240455, 240456, 240457, 240458, 240459, 240460) c6( ) c7(aW, fd, cz, aT, ae, ad); #c1(SLC2A13) c2(NP_443117) c3(proton myo-inositol cotransporter) c4(229513) c5(229514, 229515, 229516, 229517, 229518, 229519) c6( ) c7(T, eM, ff, an, en, im, lB); #c1(SLC2A13) c2(XP_011536152) c3(proton myo-inositol cotransporter isoform X4) c4(333454) c5(333455, 333456, 333457, 333458, 333459, 333460) c6( ) c7(T, eM, ff, an, en, im, lB); #c1(SLC2A14) c2(NP_001273163) c3(solute carrier family 2, facilitated glucose transporter member 14 isoform b) c4(104401, 104402, 104403, 104404, 104405, 104406, 104407) c5(104408, 104409, 104410, 104411, 104412, 104413, 104414, 104415, 104416, 104417, 104418, 104419, 104420, 104421, 104422, 104423, 104424, 104425, 104426, 104427, 104428, 104429, 104430, 104431, 104432, 104433, 104434, 104435, 104436, 104437, 104438, 104439, 104440, 104441, 104442, 104443, 104444, 104445, 104446, 104447, 104448, 104449, 104450, 104451, 104452) c6( ) c7(bL, eP, ex, bB, dG, eA, x, aT, eV, dL, K, hg, nJ, eL, af, d, vT, aW, ff, eZ, ahJ, en, g, as, t, bz, lU, rR, By, eM, nB, bE, mg, mf, m, k, iu); #c1(SLC2A14) c2(NP_001273165) c3(solute carrier family 2, facilitated glucose transporter member 14 isoform c) c4(104453) c5(104454, 104455, 104456, 104457, 104458, 104459, 104460, 104461, 104462, 104463, 104464, 104465, 104466, 104467, 104468, 104469) c6( ) c7(bL, eP, ex, bB, dG, eA, x, aT, eV, dL, K, hg, nJ, eL, af, d, vT, aW, ff, eZ, ahJ, en, g, as, t, bz, lU, rR, Qy, eM, nB, bE, mg, mf, m, k, iu); #c1(SLC2A14) c2(NP_001273166) c3(solute carrier family 2, facilitated glucose transporter member 14 isoform d) c4(104470, 104471) c5(104472, 104473, 104474, 104475, 104476, 104477) c6( ) c7(bL, eP, ex, bB, dG, eA, x, aT, eV, dL, K, hg, nJ, eL, af, d, vT, aW, ff, eZ, ahJ, en, g, as, t, bz, lU, rR, Qy, eM, nB, bE, mg, mf, m, k, iu); #c1(SLC2A14) c2(NP_703150) c3(solute carrier family 2, facilitated glucose transporter member 14 isoform a) c4(246020, 246021, 246022) c5(246023, 246024, 246025, 246026, 246027, 246028, 246029, 246030, 246031, 246032, 246033, 246034, 246035, 246036, 246037, 246038, 246039, 246040, 246041, 246042, 246043, 246044) c6( ) c7(bL, eP, ex, bB, dG, eA, x, aT, eV, dL, K, hg, nJ, eL, af, d, vT, aW, ff, eZ, ahJ, en, g, as, t, bz, lU, rR, Qy, eM, nB, bE, mg, mf, m, k, iu); #c1(SLC2A2) c2(NP_000331) c3(solute carrier family 2, facilitated glucose transporter member 2 isoform 1) c4(7196) c5(7197, 7198, 7199, 7200, 7201, 7202, 7203, 7204, 7205, 7206, 7207, 7208, 7209, 7210, 7211, 7212, 7213, 7214, 7215, 7216, 7217, 7218, 7219, 7220, 7221, 7222, 7223, 7224, 7225, 7226, 7227, 7228, 7229, 7230, 7231, 7232, 7233, 7234, 7235, 7236, 7237, 7238, 7239, 7240, 7241, 7242, 7243, 7244, 7245, 7246, 7247, 7248, 7249, 7250, 7251, 7252, 7253, 7254, 7255, 7256, 7257, 7258, 7259, 7260, 7261, 7262, 7263, 7264, 7265, 7266, 7267, 7268, 7269, 7270, 7271, 7272, 7273, 7274, 7275, 7276, 7277, 7278, 7279, 7280, 7281, 7282, 7283, 7284, 7285, 7286, 7287, 7288, 7289, 7290, 7291, 7292, 7293, 7294, 7295, 7296, 7297, 7298, 7299, 7300, 7301, 7302, 7303, 7304, 7305, 7306, 7307, 7308, 7309, 7310, 7311, 7312, 7313, 7314, 7315, 7316, 7317, 7318, 7319, 7320, 7321, 7322, 7323, 7324, 7325, 7326, 7327, 7328, 7329, 7330, 7331, 7332, 7333, 7334, 7335, 7336, 7337, 7338, 7339, 7340) c6( ) c7(bL, T, dD, fM, Uc, e, bB, aj, dG, v, ci, eq, J, mj, mh, cFt, awd, aT, Ps, bq, blX, yo, A, aW, vj, ff, Bw, fc, ahJ, lo, en, bE, Qu, bo, arC, lZ, hR, mf, dy, B, jr); #c1(SLC2A2) c2(NP_001265587) c3(solute carrier family 2, facilitated glucose transporter member 2 isoform 2) c4(98911) c5(98912, 98913, 98914, 98915, 98916, 98917, 98918) c6( ) c7(bL, T, dD, fM, Uc, e, bB, aj, dG, v, ci, eq, J, mj, mh, cFt, awd, aT, Ps, bq, blX, yo, A, aW, vj, ff, Bw, fc, ahJ, lo, en, bE, Qu, bo, arC, lZ, hR, mf, dy, B, jr); #c1(SLC2A3) c2(NP_008862) c3(solute carrier family 2, facilitated glucose transporter member 3) c4(169294, 169295) c5(169296, 169297, 169298, 169299, 169300, 169301, 169302, 169303, 169304, 169305, 169306, 169307, 169308, 169309) c6( ) c7(bL, eP, eX, cz, bB, dG, ci, eA, dP, aT, lG, eV, dL, K, hg, bq, nJ, eL, af, d, vT, aW, vj, fJ, ff, eZ, ahJ, en, g, as, t, bz, lU, rR, Qy, eM, nB, bE, bo, hr, mg, mf, Lb, m, yA, du, iu); #c1(SLC2A4RG) c2(NP_064446) c3(SLC2A4 regulator) c4(202504) c5(202505, 202506, 202507, 202508, 202509, 202510, 202511, 202512, 202513) c6( ) c7(T, jv, dD, yS, aZ, ea, ha, bS, eq, nA, eA, ad, afR, afU, aT, K, bU, afx, ae, kl, aW, Y, g, cm, nC, bz, dr, bN, ck, bF, HK, yA, bt); #c1(SLC2A5) c2(XP_011540307) c3 (solute carrier family 2, facilitated glucose transporter member 5 isoform X2) c4( ) c5(338718, 338719, 338720, 338721, 338722, 338723, 338724, 338725, 338726, 338727, 338728, 338729) c6( ) c7(bL, T, aW, ev, aT, cz, aq, ei, ae, B, ad); #c1(SLC2A6) c2(NP_001138571) c3(solute carrier family 2, facilitated glucose transporter member 6 isoform 2) c4(57017) c5(57018, 57019, 57020, 57021, 57022, 57023) c6( ) c7(nD, T, aw, eZ, fd, cES, j, nz); #c1(SLC2A6) c2(NP_060055) c3(solute carrier family 2, facilitated glucose transporter member 6 isoform 1) c4(193916, 193917) c5(193918, 193919, 193920, 193921, 193922, 193923) c6( ) c7(nD, T, aw, eZ, fd, cES, j, nz); #c1(SLC2A6) c2(XP_011516490) c3(solute carrier family 2, facilitated glucose transporter member 6 isoform X1) c4( ) c5(309888, 309889, 309890, 309891, 309892, 309893) c6( ) c7(nD, T, aw, eZ, fd, cES, j, nz); #c1(SLC2A6) c2(XP_011516491) c3(solute carrier family 2, facilitated glucose transporter member 6 isoform X2) c4(309894) c5(309895, 309896, 309897, 309898, 309899, 309900) c6( ) c7(nD, T, aw, eZ, fd, cES, j, nz); #c1(SLC2A8) c2(NP_001258640) c3(solute carrier family 2, facilitated glucose transporter member 8 isoform 2) c4(96246) c5(96247, 96248, 96249, 96250, 96251, 96252) c6( ) c7(fd); #c1(SLC2A8) c2(NP_001258641) c3(solute carrier family 2, facilitated glucose transporter member 8 isoform 3) c4( ) c5(96253, 96254, 96255, 96256, 96257, 96258, 96259, 96260, 96261, 96262, 96263) c6( ) c7(fd); #c1(SLC2A8) c2(NP_055395) c3(solute carrier family 2, facilitated glucose transporter member 8 isoform 1) c4(180760, 180761) c5(180762, 180763, 180764, 180765, 180766, 180767) c6( ) c7(fd); #c1(SLC2A8) c2(XP_006717147) c3(solute carrier family 2, facilitated glucose transporter member 8 isoform X2) c4(290782) c5( ) c6( ) c7(fd); #c1(SLC2A8) c2(XP_011516905) c3(solute carrier family 2, facilitated glucose transporter member 8 isoform X3) c4(310363, 310364, 310365) c5(310366, 310367, 310368, 310369, 310370, 310371, 310372, 310373, 310374, 310375, 310376, 310377, 310378, 310379, 310380, 310381, 310382, 310383, 310384, 310385, 310386, 310387, 310388) c6( ) c7(fd); #c1(SLC2A8) c2(XP_011516906) c3(solute carrier family 2, facilitated glucose transporter member 8 isoform X4) c4(310389, 310390) c5(310391, 310392, 310393, 310394, 310395, 310396, 310397, 310398, 310399, 310400, 310401, 310402) c6( ) c7(fd); #c1(SLC2A9) c2(NP_001001290) c3(solute carrier family 2, facilitated glucose transporter member 9 isoform 2) c4(18141) c5(18142, 18143, 18144, 18145, 18146, 18147, 18148, 18149, 18150, 18151) c6( ) c7(bL, nD, T, nH, nn, Na, cFu, QU, aw, fd, zj, nq, cES, v, j, ei, af, hq); #c1(SLC2A9) c2(NP_064425) c3(solute carrier family 2, facilitated glucose transporter member 9 isoform 1) c4(202422) c5(202423, 202424, 202425, 202426, 202427, 202428) c6( ) c7(bL, nD, T, nH, nn, Na, cFu, QU, aw, fd, zj, nq, cES, v, j, ei, af, hq); #c1(SLC2A9) c2(XP_006714032) c3(solute carrier family 2, facilitated glucose transporter member 9 isoform X13) c4(287510) c5(287511, 287512, 287513, 287514, 287515, 287516) c6( ) c7(bL, nD, T, nH, nn, Na, cFu, QU, aw, fd, zj, nq, cES, v, j, ei, af, hq); #c1(SLC30A1) c2(NP_067017) c3(zinc transporter 1) c4(208389) c5(208390, 208391, 208392, 208393, 208394, 208395) c6( ) c7(pa, mj, aE, af, mr, qt, sO, aW, wL, aT, at, cy, aq, dL, B); #c1(SLC30A10) c2(NP_061183) c3(zinc transporter 10) c4(199823, 199824) c5(199825, 199826, 199827, 199828, 199829, 199830, 199831, 199832, 199833, 199834, 199835, 199836) c6( ) c7(bL, mj, A, yG, yP, Wk, aU, aj, mf, dG, Mz, jy, J, ad, cfv); #c1(SLC30A10) c2(XP_006711500) c3(zinc transporter 10 isoform X1) c4(286040) c5(286041) c6( ) c7(bL, mj, A, yG, yP, Wk, aU, aj, mf, dG, Mz, jy, J, ad, cFv); #c1(SLC30A10) c2(XP_011508029) c3(zinc transporter 10 isoform X2) c4( ) c5(298483, 298484, 298485, 298486, 298487) c6( ) c7(bL, mj, A, yG, yP, Wk, aU, aj, mf, dG, Mz, jy, J, ad, cFv); #c1(SLC30A3) c2(NP_003450) c3(zinc transporter 3) c4(138439) c5(138440, 138441, 138442, 138443, 138444, 138445) c6( ) c7(l, aj, H, J, DF, L); #c1(SLC30A3) c2(XP_005264608) c3(zinc transporter 3 isoform X4) c4(279143) c5(279144, 279145, 279146, 279147, 279148, 279149, 279150, 279151) c6( ) c7(l, aj, H, J, DF, L); #c1(SLC30A3) c2(XP_006712163) c3(zinc transporter 3 isoform X6) c4( ) c5(286480, 286481) c6( ) c7(l, aj, H, J, DF, L); #c1(SLC30A3) c2(XP_011531403) c3(zinc transporter 3 isoform X5) c4(327460, 327461, 327462) c5(327463, 327464, 327465, 327466, 327467, 327468, 327469, 327470, 327471, 327472, 327473, 327474, 327475, 327476, 327477, 327478) c6( ) c7(l, aj, H, J, DF, L); #c1(SLC30A4) c2(XP_011520299) c3(zinc transporter 4 isoform X1) c4(315150) c5(315151, 315152, 315153, 315154, 315155, 315156) c6( ) c7(cFw, pu, Jp, aNN, lG, at, dL, zx, ae, af, ad); #c1(SLC30A5) c2(NP_075053) c3(zinc transporter 5 isoform 1) c4(213592, 213593) c5(213594, 213595, 213596, 213597, 213598, 213599) c6( ) c7(ey, ad); #c1(SLC30A5) c2(NP_076960) c3(zinc transporter 5 isoform 2) c4(214471) c5(214472, 214473, 214474, 214475, 214476, 214477) c6( ) c7(ey, ad); #c1(SLC30A5) c2(XP_005248626) c3(zinc transporter 5 isoform X1) c4( ) c5(268481, 268482, 268483, 268484, 268485, 268486) c6( ) c7(ey, ad); #c1(SLC30A6) c2(NP_001180442) c3(zinc transporter 6 isoform 1) c4(74074) c5(74075, 74076, 74077, 74078, 74079, 74080) c6( ) c7(af, ad); #c1(SLC30A6) c2(NP_001180443) c3(zinc transporter 6 isoform 3) c4(74081) c5(74082, 74083, 74084, 74085, 74086, 74087) c6( ) c7(af, ad); #c1(SLC30A6) c2(NP_001180444) c3(zinc transporter 6 isoform 4) c4(74088, 74089) c5(74090, 74091, 74092, 74093, 74094, 74095) c6( ) c7(af, ad); #c1(SLC30A6) c2(NP_060434) c3(zinc transporter 6 isoform 2) c4(195917) c5(195918, 195919, 195920, 195921, 195922, 195923, 195924) c6( ) c7(af, ad); #c1(SLC30A6) c2(XP_006712111) c3(zinc transporter 6 isoform X5) c4( ) c5(286412, 286413, 286414, 286415, 286416) c6( ) c7(af, ad); #c1(SLC30A6) c2(XP_011531262) c3(zinc transporter 6 isoform X2) c4(327232) c5( ) c6( ) c7(af, ad); #c1(SLC30A6) c2(XP_011531264) c3(zinc transporter 6 isoform X3) c4( ) c5(327233, 327234, 327235, 327236, 327237, 327238) c6( ) c7(af, ad); #c1(SLC30A6) c2(XP_011531265) c3(zinc transporter 6 isoform X4) c4(327239) c5(327240) c6( ) c7(af, ad); #c1(SLC30A7) c2(XP_011539081) c3(zinc transporter 7 isoform X1) c4(337147) c5(337148, 337149, 337150, 337151, 337152, 337153) c6( ) c7(V, it, ht, ad); #c1(SLC30A8) c2(NP_001166286) c3(zinc transporter 8 isoform b) c4( ) c5(68543, 68544, 68545, 68546, 68547) c6( ) c7(bL, bM, dD, aw, yG, jx, aj, dG, j, v, eq, J, ad, mj, ga, h, jy, kl, A, zR, Mz, yP, mf, lS); #c1(SLC30A8) c2(XP_011515183) c3(zinc transporter 8 isoform X2) c4(308016, 308017) c5(308018, 308019, 308020, 308021, 308022, 308023, 308024) c6( ) c7(bL, bM, dD, aw, yG, jx, aj, dG, j, v, eq, J, ad, mj, ga, h, jy, kl, A, zR, Mz, yP, mf, lS); #c1(SLC30A9) c2(NP_006336) c3(zinc transporter 9) c4(164132, 164133) c5(164134, 164135, 164136, 164137, 164138, 164139, 164140, 164141, 164142, 164143, 164144, 164145) c6( ) c7(ad); #c1(SLC31A1) c2(NP_001850) c3(high affinity copper uptake protein 1) c4(121663, 121664) c5(121665, 121666, 121667, 121668, 121669, 121670, 121671, 121672, 121673, 121674, 121675, 121676) c6( ) c7(eY, ae, T, eM, eL, JT, QP, dL, m, nA, t, bz, ad); #c1(SLC31A2) c2(NP_001851) c3(probable low affinity copper uptake protein 2) c4(121677) c5(121678, 121679, 121680, 121681, 121682, 121683) c6( ) c7(T, nv, fd, K, m, t); #c1(SLC32A1) c2(NP_542119) c3(vesicular inhibitory amino acid transporter) c4(231389) c5(231390, 231391, 231392, 231393, 231394, 231395) c6( ) c7(ha, s); #c1(SLC33A1) c2(NP_001177921) c3(acetyl-coenzyme A transporter 1) c4(73162) c5(73163, 73164, 73165, 73166, 73167, 73168, 73169, 73170, 73171, 73172) c6( ) c7(bL, aiZ, dD, Pt, aZ, ea, e, aw, cz, cQ, bS, j, ct, Vd, Lw, v, eq, cd, ad, dP, fU, F, aE, wG, lu, M, aT, vc, K, Fg, cFy, ae, vP, eu, tw, s, aW, Y, aq, uU, Hu, Z, Mu, qa, Qn, ek, cFx, kT, c, U, MM, MJ, dy, B); #c1(SLC34A1) c2(NP_001161051) c3(sodium-dependent phosphate transport protein 2A isoform 2) c4(65229, 65230) c5(65231, 65232, 65233, 65234, 65235, 65236, 65237, 65238, 65239, 65240, 65241, 65242) c6( ) c7(fU, cFA, AB, Tl, eR, ig, cFz, bbM, aAP, gT, awd, hr, cFC, lG, dy, cFB, azh); #c1(SLC34A1) c2(NP_003043) c3(sodium-dependent phosphate transport protein 2A isoform 1) c4(134634) c5(134635, 134636, 134637, 134638, 134639, 134640) c6( ) c7(fU, cFA, AB, Tl, eR, ig, cFz, bbM, aAP, gT, awd, hr, cFC, lG, dy, cFB, azh); #c1(SLC34A2) c2(NP_001171470) c3(sodium-dependent phosphate transport protein 28 isoform b) c4(70209, 70210) c5(70211, 70212, 70213, 70214, 70215, 70216, 70217, 70218, 70219, 70220, 70221, 70222, 70223, 70224, 70225, 70226) c6( ) c7(eM, fV, bsW, bzo, bta, bsS, cFD); #c1(SLC34A2) c2(NP_006415) c3(sodium-dependent phosphate transport protein 28 isoform a) c4(164823, 164824) c5(164825, 164826, 164827, 164828, 164829, 164830, 164831, 164832, 164833, 164834, 164835, 164836) c6( ) c7(eM, fV, bsW, bzo, bta, bsS, cFD); #c1(SLC34A3) c2(NP_001170788) c3(sodium-dependent phosphate transport protein 2C) c4(69536) c5(69537, 69538, 69539, 69540, 69541, 69542, 69543, 69544, 69545, 69546) c6( ) c7(amB, cFA, Tl, Ay, lZ, dq, cFE, cEF, ars, AA, aAN, cFF, aLl); #c1(SLC34A3) c2(XP_011516564) c3(sodium-dependent phosphate transport protein 2C isoform X2) c4( ) c5(309995) c6( ) c7(amB, cFA, Tl, Ay, lZ, dq, cFE, cEF, ars, AA, aAN, cFF, aLl); #c1(SLC35A1) c2(NP_001161870) c3(CMP-sialic acid transporter isoform b) c4(66224, 66225, 66226) c5(66227, 66228, 66229, 66230, 66231, 66232, 66233, 66234, 66235, 66236, 66237, 66238, 66239, 66240, 66241, 66242, 66243, 66244, 66245, 66246, 66247, 66248, 66249) c6( ) c7(aAV, eb, Km, Lo, K, cFG); #c1(SLC35A1) c2(NP_006407) c3(CMP-sialic acid transporter isoform a) c4(164765, 164766, 164767) c5(164768, 164769, 164770, 164771, 164772, 164773, 164774, 164775, 164776, 164777, 164778, 164779, 164780) c6( ) c7(aAV, eb, Km, Lo, K, cFG); #c1(SLC35A2) c2(NP_001027460) c3(UDP-galactose translocator isoform b) c4(30222) c5(30223, 30224, 30225, 30226, 30227, 30228) c6( ) c7(T, qo, pf, ha, ht, bB, q, ad, hX, cFH, byF, dv, aT, dL, lm, hu, ae, eL, PE, aW, hC, bqy, ff, r, aq, en, cl, bE, hL, Km, B, EB); #c1(SLC35A2) c2(NP_001035963) c3(UDP-galactose translocator isoform c) c4(33556, 33557) c5(33558, 33559, 33560, 33561, 33562, 33563, 33564, 33565, 33566, 33567, 33568, 33569) c6( ) c7(T, qo, pf, ha, ht, bB, q, ad, hX, cFH, byF, dv, aT, dL, lm, hu, ae, eL, PE, aW, hC, bqy, ff, r, aq, en, cl, bE, hL, Km, B, EB); #c1(SLC35A2) c2(NP_001269576) c3(UDP-galactose translocator isoform d) c4(102026) c5(102027, 102028, 102029, 102030, 102031, 102032, 102033, 102034, 102035, 102036, 102037, 102038) c6( ) c7(T, qo, pf, ha, ht, bB, q, ad, hX, cFH, byF, dv, aT, dL, lm, hu, ae, eL, PE, aW, hC, bqy, ff, r, aq, en, cl, bE, hL, Km, B, EB); #c1(SLC35A2) c2(NP_001269577) c3(UDP-galactose translocator isoform e) c4(102039) c5(102040, 102041, 102042, 102043, 102044, 102045) c6( ) c7(T, qo, pf, ha, ht, bB, q, ad, hX, cFH, byF, dv, aT, dl, lm, hu, ae, eL, PE, aW, hC, bqy, ff, r, aq, en, cl, bE, hL, Km, B, EB); #c1(SLC35A2) c2(NP_001269578) c3(UDP-galactose translocator isoform f) c4(102046) c5(102047, 102048, 102049, 102050, 102051, 102052, 102053) c6( ) c7(T, qo, pf, ha, ht, bB, q, ad, hX, cFH, byF, dv, aT, dL, lm, hu, ae, eL, PE, aW, hC, bqy, ff, r, aq, en, cl, bE, hL, Km, B, EB); #c1(SLC35A2) c2(NP_001269580) c3(UDP-galactose translocator isoform h) c4(102054) c5(102055, 102056, 102057, 102058, 102059, 102060) c6( ) c7(T, qo, pf, ha, ht, bB, q, ad, hX, cFH, byF, dv, aT, dL, lm, hu, ae, eL, PE, aW, hC, bqy, ff, r, aq, en, cl, bE, hL, Km, B, EB); #c1(SLC35A2) c2(NP_005651) c3(UDP-galactose translocator isoform a) c4(158286, 158287) c5(158288, 158289, 158290, 158291, 158292, 158293) c6( ) c7(T, qo, pf, ha, ht, bB, q, ad, hX, cFH, byF, dv, aT, dL, lm, hu, ae, eL, PE, aW, hC, bqy, ff, r, aq, en, cl, bE, hL, Km, B, EB); #c1(SLC35A3) c2(XP_011539439) c3(UDP-N-acetylglucosamine transporter isoform X3) c4( ) c5(337598, 337599, 337600, 337601, 337602, 337603) c6( ) c7(cFl, W, af); #c1(SLC35A3) c2(XP_011539440) c3(UDP-N-acetylglucosamine transporter isoform X4) c4(337604) c5(337605) c6( ) c7(cFl, W, af); #c1(SLC35A3) c2(XP_011539441) c3(UDP-N-acetylglucosamine transporter isoform X5) c4( ) c5(337606, 337607, 337608, 337609, 337610, 337611) c6( ) c7(cFl, W, af); #c1(SLC35A4) c2(NP_542401) c3(probable UDP-sugar transporter protein SLC35A4) c4(232351) c5(232352, 232353, 232354, 232355, 232356, 232357, 232358, 232359, 232360, 232361, 232362, 232363, 232364, 232365) c6( ) c7(T, B, aW, aT, aF, iu); #c1(SLC35B2) c2(NP_001273442) c3(adenosine 3′-phospho 5′-phosphosulfate transporter 1 isoform d precursor) c4(104896) c5(104897, 104898, 104899, 104900, 104901, 104902) c6( ) c7(T, fM, TY, iW, Of, jh, cj, Og, Od, cFJ); #c1(SLC35B2) c2(NP_001273446) c3(adenosine 3′-phospho 5′-phosphosulfate transporter 1 isoform e) c4( ) c5(104924) c6( ) c7(T, fM, TY, iW, Of, jh, cj, Og, Od, cFJ); #c1(SLC35B2) c2(NP_001273448) c3(adenosine 3′-phospho 5′-phosphosulfate transporter 1 isoform f) c4(104925) c5(104926, 104927, 104928, 104929, 104930) c6( ) c7(T, fM, TY, iW, Of, jh, cj, Og, Od, cFJ); #c1(SLC35B2) c2(NP_835361) c3(adenosine 3′-phospho 5′-phosphosulfate transporter 1 isoform a precursor) c4(253461, 253462) c5(253463, 253464, 253465, 253466, 253467, 253468, 253469, 253470, 253471, 253472, 253473, 253474, 253475) c6( ) c7(T, fM, TY, iW, Of, jh, cj, Og, Od, cFJ); #c1(SLC35B4) c2(NP_116215) c3(UDP-xylose and UDP-N-acetylglucosamine transporter) c4(225816) c5(225817, 225818, 225819, 225820, 225821, 225822) c6( ) c7(v); #c1(SLC35C1) c2(NP_001138738) c3(GDP-fucose transporter 1 isoform b) c4(57502) c5(57503, 57504, 57505, 57506, 57507, 57508, 57509, 57510) c6( ) c7(dr, B, Km); #c1(SLC35C1) c2(NP_060859) c3(GDP-fucose transporter 1 isoform a) c4(198129, 198130, 198131) c5(198132, 198133, 198134, 198135, 198136, 198137, 198138, 198139, 198140, 198141, 198142, 198143, 198144, 198145) c6( ) c7(dr, B, Km); #c1(SLC35C1) c2(XP_011518505) c3(GDP-fucose transporter 1 isoform X2) c4(312589) c5(312590, 312591, 312592, 312593, 312594, 312595, 312596, 312597, 312598, 312599, 312600, 312601) c6( ) c7(dr, B, Km); #c1(SLC35D3) c2(NP_001008783) c3(solute carrier family 35 member D3) c4(23416) c5(23417, 23418, 23419, 23420, 23421, 23422) c6( ) c7(v, lF, ei); #c1(SLC35F1) c2(NP_001025029) c3(solute carrier family 35 member F1) c4(29232) c5(29233, 29234, 29235, 29236, 29237, 29238) c6( ) c7(e, oe, cd, fq); #c1(SLC35F2) c2(NP_059985) c3(solute carrier family 35 member F2) c4(193484, 193485) c5(193486, 193487, 193488, 193489, 193490, 193491, 193492, 193493, 193494, 193495, 193496, 193497, 193498, 193499, 193500, 193501, 193502) c6( ) c7(j, ac); #c1(SLC35F2) c2(XP_005271655) c3(solute carrier family 35 member F2 isoform X1) c4(283515, 283516) c5(283517, 283518, 283519, 283520, 283521, 283522, 283523, 283524, 283525, 283526, 283527) c6( ) c7(j, ac); #c1(SLC35F3) c2(NP_001287774) c3(putative thiamine transporter SLC35F3 isoform 2) c4(112848) c5(112849, 112850, 112851, 112852, (12853, 112854) c6( ) c7(ac, cCU, aw, cz, gv, K, sj); #c1(SLC35F3) c2(NP_775779) c3(putative thiamine transporter SLC35F3 isoform 1) c4(250266) c5(250267, 250268, 250269, 250270, 250271, 250272) c6( ) c7(ac, cCU, aw, cz, gv, K, sj); #c1(SLC35F4) c2(XP_011535022) c3(solute carrier family 35 member F4 isoform X1) c4(331719) c5( ) c6( ) c7(bK, sj); #c1(SLC35F4) c2(XP_011535026) c3(solute carrier family 35 member F4 isoform X5) c4( ) c5(331720, 331721, 331722, 331723, 331724, 331725) c6( ) c7(bK, sj); #c1(SLC35F6) c2(NP_060347) c3(solute carrier family 35 member F6 precursor) c4(195529, 195530) c5(195531, 195532, 195533, 195534, 195535, 195536, 195537, 195538, 195539, 195540, 195541, 195542) c6( ) c7(aC, kp, aE); #c1(SLC35G1) c2(NP_001128130) c3(solute carrier family 35 member Gl isoform 1) c4(49604) c5(49605, 49606, 49607, 49608, 49609, 49610) c6( ) c7(bL, ck, T, bN, LO, jN, h, aj, bS, v, ct, pm, asA, Z, bt, ae, J, ad); #c1(SLC35G1) c2(NP_694958) c3(solute carrier family 35 member Gl isoform 2) c4(245485) c5(245486, 245487, 245488, 245489, 245490, 245491) c6( ) c7(bL, ck, T, bN, LO, jN, h, aj, bS, v, ct, pm, asA, Z, bt, ae, J, ad); #c1(SLC35G2) c2(NP_001091069) c3(solute carrier family 35 member G2) c4(38448) c5(38449, 38450, 38451, 38452, 38453, 38454, 38455, 38456, 38457, 38458) c6( ) c7(Y, fr); #c1(SLC35G5) c2(NP_473369) c3(solute carrier family 35 member G5) c4(230465) c5(230466, 230467, 230468, 230469, 230470, 230471, 230472) c6( ) c7(c0, bL, nX); #c1(SLC35G6) c2(NP_001096084) c3(solute carrier family 35 member GG) c4(41064) c5(41065, 41066, 41067, 41068, 41069, 41070) c6( ) c7(aT); #c1(SLC36A1) c2(NP_510968) c3(proton-coupled amino acid transporter 1) c4( ) c5(231100, 231101, 231102, 231103, 231104, 231105, 231106, 231107) c6( ) c7(aW, cz, ad, yw, al); #c1(SLC36A1) c2(XP_011535882) c3(proton-coupled amino acid transporter 1 isoform X2) c4(333152, 333153, 333154, 333155, 333156) c5(333157, 333158) c6( ) c7(aW, cz, ad, yw, aT); #c1(SLC36A1) c2(XP_011535897) c3(proton-coupled amino acid transporter 1 isoform X4) c4( ) c5(333159, 333160) c6( ) c7(aW, cz, ad, yw, aT); #c1(SLC36A1) c2(XP_011535898) c3(proton-coupled amino acid transporter 1 isoform X6) c4( ) c5(333161, 333162) c6( ) c7(aW, cz, ad, yw, aT); #c1(SLC36A2) c2(NP_861441) c3(proton-coupled amino acid transporter 2) c4(255572) c5(255573, 255574, 255575, 255576, 255577, 255578, 255579) c6( ) c7(cFK, fq, cFL, hp); #c1(SLC36A2) c2(XP_006714819) c3(proton-coupled amino acid transporter 2 isoform X1) c4(288249) c5(288250, 288251, 288252, 288253, 288254, 288255) c6( ) c7(cFK, fq, cFL, hp); #c1(SLC37A1) c2(NP_061837) c3(glycerol-3-phosphate transporter) c4(200709, 200710) c5(200711, 200712, 200713, 200714, 200715, 200716, 200717, 200718, 200719, 200720, 200721, 200722, 200723, 200724, 200725, 200726, 200727, 200728, 200729, 200730) c6( ) c7(cFM, aT); #c1(SLC37A1) c2(XP_011527917) c3(glycerol-3-phosphate transporter isoform X3) c4( ) c5(323017) c6( ) c7(cFM, al); #c1(SLC37A2) c2(NP_001138762) c3(sugar phosphate exchanger 2 isoform 2) c4(57588) c5(57589, 57590, 57591, 57592, 57593, 57594) c6( ) c7(v); #c1(SLC37A2) c2(NP_938018) c3(sugar phosphate exchanger 2 isoform 1) c4(259526) c5(259527, 259528, 259529, 259530, 259531, 259532, 259533, 259534, 259535, 259536) c6( ) c7(v); #c1(SLC37A2) c2(XP_011540963) c3(sugar phosphate exchanger 2 isoform X1) c4(339498) c5(339499, 339500, 339501, 339502, 339503, 339504) c6( ) c7(v); #c1(SLC37A4) c2(NP_001157749) c3(glucose-6-phosphate translocase isoform 1) c4(62539) c5(62540, 62541, 62542, 62543, 62544, 62545, 62546, 62547, 62548, 62549, 62550, 62551, 62552, 62553, 62554, 62555, 62556, 62557, 62558, 62559, 62560, 62561, 62562, 62563, 62564, 62565, 62566, 62567, 62568, 62569, 62570, 62571, 62572, 62573, 62574, 62575, 62576, 62577, 62578, 62579, 62580, 62581, 62582, 62583, 62584, 62585, 62586, 62587, 62588, 62589, 62590, 62591, 62592, 62593, 62594, 62595, 62596, 62597, 62598, 62599, 62600, 62601, 62602, 62603, 62604, 62605, 62606, 62607, 62608, 62609, 62610, 62611, 62612, 62613, 62614, 62615, 62616, 62617, 62618, 62619, 62620, 62621, 62622, 62623, 62624, 62625, 62626, 62627, 62628, 62629, 62630, 62631) c6( ) c7(gV, T, mO, d, A, byo, hG, dB, Bw, fQ, dr, afK, aj, EZ, cEG, AE, ex, bq, eA, J); #c1(SLC37A4) c2(NP_001157750) c3(glucose-6-phosphate translocase isoform 2) c4(62632) c5(62633, 62634, 62635, 62636, 62637, 62638) c6( ) c7(gV, T, mD, d, A, byo, hG, dB, Bw, fQ, dr, afK, aj, EZ, cEG, AE, ex, bq, eA, J); #c1(SLC38A1) c2(NP_001265316) c3(sodium-coupled neutral amino acid transporter 1 isoform 1) c4(98240) c5(98241, 98242, 98243, 98244, 98245, 98246, 98247, 98248, 98249, 98250, 98251, 98252, 98253, 98254, 98255, 98256, 98257, 98258) c6( ) c7(azs, T, bM, bO, fd, bB, x, gs, el, aW, NR, g, ba, eM, nB, cX, bcl, k, m, NT, n, c, aj, fr, cd, amJ, h, dL, hB, aDX, fe, hq, Qy, at, it, B, bL, bll, aA, cz, q, J, eA, XX, nv, aT, bK, sX, ae, bkl, avV, s, nO, bg, r, eS, en, cT, vK, il, ig, MB, ciA, bt, blu, hS, gx, iW, ha, jh, bS, ad, KL, hX, dP, l, hd, NS, oW, io, BO, ff, eZ, Ej, aF, bz, bF, apM, bE, X, pP, bC, i, hE, iu, y); #c1(SLC38A2) c2(NP_061849) c3(sodium-coupled neutral amino acid transporter 2) c4(200771) c5(200772, 200773, 200774, 200775, 200776, 200777) c6( ) c7(T, s, aW, Y, ha, aT, aA, LA, ey, ci, K, Dz, g, B, ad); #c1(SLC38A4) c2(NP_001137296) c3(sodium-coupled neutral amino acid transporter 4) c4(55335, 55336, 55337, 55338) c5(55339, 55340, 55341, 55342, 55343, 55344, 55345, 55346, 55347, 55348, 55349, 55350, 55351, 55352, 55353, 55354, 55355, 55356, 55357, 55358, 55359, 55360, 55361, 55362, 55363, 55364, 55365, 55366) c6( ) c7(aw, aj, dG, B, ad); #c1(SLC38A5) c2(NP_277053) c3(sodium-coupled neutral amino acid transporter 5) c4(228836, 228837) c5(228838, 228839, 228840, 228841, 228842, 228843, 228844, 228845, 228846, 228847, 228848, 228849, 228850, 228851, 228852, 228853, 228854, 228855, 228856, 228857, 228858, 228859, 228860, 228861, 228862, 228863, 228864, 228865, 228866, 228867, 228868, 228869, 228870, 228871, 228872, 228873, 228874, 228875, 228876, 228877, 228878, 228879, 228880) c6( ) c7(nA); #c1(SLC38A5) c2(XP_005272751) c3(sodium-coupled neutral amino acid transporter 5 isoform X2) c4( ) c5(284176, 284177) c6( ) c7(nA); #c1(SLC38A5) c2(XP_005272754) c3(sodium-coupled neutral amino acid transporter 5 isoform X3) c4(284178, 284179, 284180, 284181, 284182) c5(284183) c6( ) c7(nA); #c1(SLC38A6) c2(NP_001166173) c3(probable sodium-coupled neutral amino acid transporter 6 isoform 1) c4(68414, 68415, 68416) c5(68417, 68418, 68419, 68420, 68421, 68422, 68423, 68424, 68425, 68426, 68427, 68428, 68429, 68430, 68431, 68432, 68433, 68434) c6( ) c7(gx, azs, T, iW, aA, bB, j, q, ad, av, aE, amJ, aT, dL, j, Od, ae, eL, bkl, avV, s, aW, ff, r, af, en, cm, ik, bz, fq, cT, apM, bE, B, blu); #c1(SLC38A6) c2(NP_722518) c3(probable sodium-coupled neutral amino acid transporter 6 isoform 2) c4(246779) c5(246780, 246781, 246782, 246783, 246784, 246785) c6( ) c7(gx, azs, T, iW, aA, bB, j, q, ad, av, aE, amJ, aT, dL, j, Od, ae, eL, bkl, avV, s, aW, ff, r, aF, en, cm, ik, bz, fq, cT, apM, bE, B, blu); #c1(SLC38A7) c2(NP_060701) c3 (putative sodium-coupled neutral amino acid transporter 7) c4(197293) c5(197294, 197295, 197296, 197297, 197298, 197299) c6( ) c7(agd, beo, eM, nO, aW, cER, wS, akr, bO, aT, ff, ti, cFK, v, aXd, K, bC, en, he, cFL, qa); #c1(SLC38A8) c2(NP_001073911) c3 (putative sodium-coupled neutral amino acid transporter 8) c4(36498) c5(36499, 36500, 36501, 36502, 36503, 36504, 36505) c6( ) c7(KE, coq, cFN, cFO); #c1(SLC38A9) c2(NP_001245215) c3(putative sodium-coupled neutral amino acid transporter 9 isoform 2) c4( ) c5(92731, 92732, 92733, 92734, 92735, 92736) c6( ) c7(cm, aq); #c1(SLC38A9) c2(NP_001245216) c3(putative sodium-coupled neutral amino acid transporter 9 isoform 3) c4(92737) c5(92738, 92739, 92740, 92741, 92742, 92743) c6( ) c7(cm, aq); #c1(SLC38A9) c2(NP_001269358) c3(putative sodium-coupled neutral amino acid transporter 9 isoform 4) c4( ) c5(101270, 101271, 101272, 101273, 101274) c6( ) c7(cm, aq); #c1(SLC38A9) c2(NP_775785) c3(putative sodium-coupled neutral amino acid transporter 9 isoform 1) c4(250280, 250281, 250282, 250283, 250284, 250285) c5(250286, 250287, 250288, 250289, 250290, 250291, 250292, 250293, 250294, 250295, 250296, 250297, 250298, 250299, 250300, 250301) c6( ) c7(cm, aq); #c1(SLC38A9) c2(XP_011541478) c3(putative sodium-coupled neutral amino acid transporter 9 isoform X3) c4(340065) c5( ) c6( ) c7(cm, aq); #c1(SLC38A9) c2(XP_011541481) c3 (putative sodium-coupled neutral amino acid transporter 9 isoform X6) c4(340066) c5( ) c6( ) c7(cm, aq); #c1(SLC38A9) c2(XP_011541484) c3(putative sodium-coupled neutral amino acid transporter 9 isoform X8) c4( ) c5(340067, 340068, 340069, 340070, 340071, 340072) c6( ) c7(cm, aq); #c1(SLC38A9) c2(XP_011541486) c3(putative sodium-coupled neutral amino acid transporter 9 isoform X10) c4( ) c5(340073, 340074, 340075, 340076, 340077, 340078, 340079, 340080, 340081, 340082, 340083, 340084, 340085, 340086, 340087, 340088, 340089, 340090, 340091, 340092, 340093, 340094, 340095) c6( ) c7(cm, aq); #c1(SLC39A1) c2(NP_001258888) c3(zinc transporter ZIP1 isoform a) c4(96654, 96655, 96656, 96657, 96658, 96659) c5(96660, 96661, 96662, 96663, 96664, 96665, 96666, 96667, 96668, 96669, 96670, 96671, 96672, 96673, 96674, 96675, 96676, 96677, 96678, 96679, 96680, 96681, 96682, 96683, 96684, 96685, 96686, 96687, 96688, 96689, 96690, 96691, 96692, 96693, 96694, 96695, 96696, 96697, 96698, 96699, 96700, 96701, 96702, 96703, 96704, 96705, 96706, 96707) c6( ) c7(b, dD, ea, Jp, ao, eq, i, ae, ad); #c1(SLC39A10) c2(NP_001120729) c3(zinc transporter ZIP10 precursor) c4(44723, 44724, 44725, 44726, 44727) c5(44728, 44729, 44730, 44731, 44732, 44733, 44734, 44735, 44736, 44737, 44738, 44739, 44740, 44741, 44742, 44743, 44744, 44745, 44746, 44747, 44748) c6( ) c7(yw, yt, aW, aT, Ea, cm, ad); #c1(SLC39A10) c2(XP_011509809) c3(zinc transporter ZIP10 isoform X2) c4( ) c5(300362, 300363, 300364, 300365, 300366, 300367, 300368, 300369, 300370, 300371, 300372, 300373, 300374) c6( ) c7(yw, yt, aW, aT, Ea, cm, ad); #c1(SLC39A11) c2(NP_001153242) c3(zinc transporter ZIP11 isoform 1) c4(60558) c5(60559, 60560, 60561, 60562, 60563, 60564) c6( ) c7(oe, c); #c1(SLC39A11) c2(NP_631916) c3(zinc transporter ZIP11 isoform 2) c4(238328) c5(238329, 238330, 238331, 238332, 238333, 238334) c6( ) c7(oe, c); #c1(SLC39A12) c2(NP_001138667) c3(zinc transporter ZIP12 isoform 1 precursor) c4(57321, 57322) c5(57323, 57324, 57325, 57326, 57327, 57328, 57329, 57330, 57331, 57332, 57333, 57334, 57335) c6( ) c7(fe, Y, oe, ad, cz); #c1(SLC39A12) c2(NP_001269662) c3(zinc transporter ZIP12 isoform 3 precursor) c4(102454) c5(102455, 102456, 102457, 102458, 102459, 102460) c6( ) c7(fe, Y, oe, ad, cz); #c1(SLC39A12) c2(NP_001269663) c3(zinc transporter ZIP12 isoform 4) c4(102461) c5(102462, 102463, 102464, 102465, 102466) c6( ) c7(fe, Y, oe, ad, cz); #c1(SLC39A12) c2(NP_689938) c3(zinc transporter ZIP12 isoform 2 precursor) c4(244401) c5(244402, 244403, 244404, 244405, 244406, 244407) c6( ) c7(fe, Y, De, ad, cz); #c1(SLC39A13) c2(NP_001121697) c3(zinc transporter ZIP13 isoform a precursor) c4( ) c5(46091, 46092, 46093, 46094, 46095, 46096) c6( ) c7(uo, cFP, Eh, ad, zw); #c1(SLC39A13) c2(NP_689477) c3(zinc transporter ZIP13 isoform b precursor) c4( ) c5(242824, 242825, 242826, 242827, 242828, 242829) c6( ) c7(uo, cFP, Eh, ad, zw); #c1(SLC39A13) c2(XP_006718444) c3(zinc transporter ZIP13 isoform X3) c4(291874) c5( ) c6( ) c7(uo, cFP, Eh, ad, zw); #c1(SLC39A13) c2(XP_011518770) c3(zinc transporter ZIP13 isoform X1) c4(313173) c5( ) c6( ) c7(uo, cFP, Eh, ad, zw); #c1(SLC39A14) c2(NP_001121903) c3(zinc transporter ZIP14 isoform a precursor) c4( ) c5(46328, 46329, 46330, 46331, 46332, 46333, 46334, 46335) c6( ) c7(T, alX, s, Jp, bo, r, fe, abX, KM, q); #c1(SLC39A14) c2(NP_001128626) c3(zinc transporter ZIP14 isoform c precursor) c4(50368) c5(50369, 50370, 50371, 50372, 50373, 50374) c6( ) c7(T, alX, s, Jp, bo, r, fe, abX, KM, q); #c1(SLC39A14) c2(NP_056174) c3(zinc transporter ZIP14 isoform b precursor) c4(186130) c5(186131, 186132, 186133, 186134, 186135, 186136) c6( ) c7(T, alX, s, Jp, bo, r, fe, abX, KM, q); #c1(SLC39A14) c2(XP_005273520) c3(zinc transporter ZIP14 isoform X3) c4(284666) c5(284667, 284668) c6( ) c7(T, alX, s, Jp, bo, r, fe, abX, KM, q); #c1(SLC39A2) c2(NP_055394) c3(zinc transporter ZIP2 isoform a) c4(180753) c5(180754, 180755, 180756, 180757, 180758, 180759) c6( ) c7(bM, dY, aT, vr, at, dL, el); #c1(SLC39A3) c2(NP_653165) c3(zinc transporter ZIP3 isoform a) c4(238893, 238894) c5(238895, 238896, 238897, 238898, 238899, 238900, 238901, 238902, 238903, 238904, 238905, 238906) c6( ) c7(T, aE, gl, bK, g, eJ); #c1(SLC39A3) c2(NP_998733) c3(zinc transporter ZIP3 isoform b) c4(265797) c5(265798, 265799, 265800, 265801, 265802, 265803) c6( ) c7(T, aE, gl, bK, g, eJ); #c1(SLC39A4) c2(NP_060237) c3(zinc transporter ZIP4 isoform 1) c4(194904) c5(194905, 194906, 194907, 194908, 194909, 194910) c6( ) c7(cFw, xd, T, aC, aE, lp, Jp, zx, ae, B, dr); #c1(SLC39A4) c2(NP_570901) c3(zinc transporter ZIP4 isoform 2 precursor) c4(234401) c5(234402, 234403, 234404, 234405, 234406, 234407) c6( ) c7(cFw, xd, T, aC, aE, lp, Jp, zx, ae, B, dr); #c1(SLC39A6) c2(NP_001092876) c3(zinc transporter ZIP6 isoform 2) c4(39693, 39694) c5(39695, 39696, 39697, 39698, 39699, 39700, 39701, 39702, 39703, 39704) c6( ) c7(T, aE, qt, aW, anV, aT, ao, n, aq, it, ci, iR, ae, B, ad); #c1(SLC39A6) c2(NP_036451) c3(zinc transporter ZIP6 isoform 1 precursor) c4( ) c5(173963, 173964, 173965, 173966, 173967, 173968, 173969, 173970) c6( ) c7(T, aE, qt, aW, anV, aT, ao, n, aq, it, ci, iR, ae, B, ad); #c1(SLC39A6) c2(XP_011524203) c3(zinc transporter ZIP6 isoform X1) c4(318735) c5( ) c6( ) c7(T, aE, qt, aW, anV, aT, ao, n, aq, it, ci, iR, ae, B, ad); #c1(SLC39A7) c2(NP_001070984) c3(zinc transporter SLC39A7 isoform 1 precursor) c4(35157, 35158) c5(35159, 35160, 35161, 35162, 35163, 35164, 35165, 35166, 35167, 35168, 35169, 35170, 35171, 35172, 35173, 35174, 35175, 35176, 35177, 35178, 35179, 35180, 35181, 35182, 35183, 35184, 35185, 35186, 35187, 35188, 35189, 35190, 35191, 35192, 35193, 35194, 35195, 35196, 35197, 35198, 35199, 35200, 35201, 35202, 35203, 35204, 35205, 35206, 35207, 35208, 35209, 35210, 35211, 35212, 35213, 35214, 35215, 35216, 35217, 35218, 35219, 35220, 35221, 35222, 35223, 35224, 35225, 35226, 35227, 35228, 35229, 35230, 35231, 35232, 35233, 35234, 35235, 35236, 35237, 35238, 35239, 35240) c6( ) c7(ac, aW, e, aw, eZ, aT, j, eu); #c1(SLC39A7) c2(NP_001275706) c3(zinc transporter SLC39A7 isoform 2) c4(107129, 107130) c5(107131, 107132, 107133, 107134, 107135, 107136, 107137, 107138, 107139, 107140, 107141, 107142, 107143, 107144, 107145, 107146, 107147, 107148, 107149, 107150, 107151) c6( ) c7(ac, aW, e, aw, eZ, aT, j, eu); #c1(SLC39A8) c2(NP_001128618) c3(zinc transporter ZIP8 isoform a precursor) c4(50350) c5(50351, 50352, 50353, 50354, 50355, 50356, 50357, 50358, 50359, 50360) c6( ) c7(dD, ea, oe, r, cz, h, j, eq, ey, aY, fq, q, ad); #c1(SLC39A8) c2(NP_001128619) c3(zinc transporter ZIP8 isoform b precursor) c4(50361) c5(50362, 50363, 50364, 50365, 50366, 50367) c6( ) c7(dD, ea, oe, r, cz, h, j, eq, ey, aY, fq, q, ad); #c1(SLC39A8) c2(XP_011530483) c3(zinc transporter ZIP8 isoform X2) c4( ) c5(326385) c6( ) c7(dD, ea, oe, r, cz, h, j, eq, ey, aY, fq, q, ad); #c1(SLC39A9) c2(NP_001239079) c3(zinc transporter ZIP9 isoform 3) c4(88253) c5(88254, 88255, 88256, 88257, 88258, 88259) c6( ) c7(aW, ae, ad, aT); #c1(SLC39A9) c2(NP_060845) c3(zinc transporter ZIP9 isoform 1) c4(198099) c5(198100, 198101, 198102, 198103, 198104, 198105, 198106, 198107, 198108, 198109) c6( ) c7(aW, ae, ad, al); #c1(SLC3A1) c2(NP_000332) c3(neutral and basic amino acid transport protein rBAT) c4(7341, 7342, 7343) c5(7344, 7345, 7346, 7347, 7348, 7349, 7350, 7351, 7352, 7353, 7354, 7355, 7356, 7357, 7358, 7359, 7360, 7361) c6( ) c7(beo, eR, tb, cW, azB, Bw, cz, vc, ww, j, ey, cFQ, azC, qa, cFR, hq); #c1(SLC3A2) c2(NP_001012680) c3(4F2 cell-surface antigen heavy chain isoform b) c4(25165, 25166, 25167, 25168, 25169) c5(25170, 25171, 25172, 25173, 25174, 25175, 25176, 25177, 25178, 25179, 25180, 25181, 25182, 25183) c6( ) c7(bM, T, Ap, ea, fd, bgs, ame, ci, Qp, aY, bgM, x, bv, boB, K, bU, hu, pk, aDU, lZ, xQ, Pf, aAg, aW, az, apn, cFU, Au, eE, ou, y, iA, iT, go, vA, mx, aep, cj, bAD, k, m, R, hh, BV, dD, akc, QO, Or, aw, yG, ht, n, oY, fr, nA, Tl, cd, ai, tD, Oe, boz, b, cq, pe, xc, cc, ep, h, jy, eV, hn, aXd, ey, Nr, OV, yi, mR, beo, A, Cu, zb, cFT, lb, Ad, fe, po, Og, yq, CQ, aYo, aKv, bfW, Wt, op, asE, kT, dB, lf, bm, SZ, FL, eo, dl, bD, Wl, bL, Ob, aZA, fM, eR, jx, eX, aA, cz, Wa, V, pd, q, zn, bjA, aT, kf, cy, qU, atw, aWs, rO, KU, ae, aoL, hM, amx, iU, oV, ev, r, akB, chy, aom, hf, t, Vk, ho, pb, ik, vk, ck, cT, cCl, cl, OW, c, nM, iQ, hc, dy, ij, bt, Ze, chP, vi, BD, bre, Ke, Pt, aD, iW, oA, asa, aHv, jh, eq, fu, hF, tK, mC, aoH, chE, F, fX, py, ds, qh, oj, hD, chX, vc, ji, fb, chN, nJ, iL, apA, yF, uQ, MG, pq, aqa, qZ, xs, Of, cr, eZ, aF, hp, app, jg, iV, iR, YZ, bji, KM, Nq, es, bF, amS, cFS, eB, gZ, aS, DO, bC, i, lC, hv); #c1(SLC3A2) c2(NP_001012682) c3(4F2 cell-surface antigen heavy chain isoform e) c4(25184) c5(25185, 25186, 25187, 25188, 25189, 25190, 25191) c6( ) c7(bM, T, Ap, ea, fd, bgs, ame, ci, Qp, aY, bgM, x, bv, boB, K, bU, hu, pk, aDU, lZ, xQ, Pf, aAg, aW, az, apn, cFU, Au, eE, ou, y, iA, iT, go, vA, mx, aep, cj, bAD, k, m, R, hh, BV, dD, akc, QO, Or, aw, yG, ht, n, oY, fr, nA, Tl, cd, ai, tD, Oe, boz, b, cq, pe, xc, cc, ep, h, jy, eV, hn, aXd, ey, Nr, OV, yi, mR, beo, A, Cu, zb, cFT, lb, Ad, fe, po, Og, yq, CQ, aYo, akv, bfW, Wt, op, asE, kT, dB, lf, bm, SZ, fL, eo, dl, bD, Wl, bL, Ob, aZA, fM, eR, jx, eX, aA, cz, Wa, V, pd, q, zn, bjA, aT, kf, cy, qU, atw, aWs, rO, KU, ae, aoL, hM, amx, iU, oV, ev, r, akB, chy, aom, hf, t, Vk, ho, pb, iK, vk, ck, cT, cCl, cl, OW, c, nM, iQ, hc, dy, ij, bt, Ze, chP, vi, BD, bre, Ke, Pt, aD, iW, oA, asa, aHv, jh, eq, fu, hF, tK, mC, aoH, chE, F, fX, py, ds, qh, oj, hD, chX, vc, ji, fb, chN, nJ, iL, apA, yF, uQ, MG, pq, aqa, qZ, xs, Of, cr, eZ, aF, hp, app, jg, iV, iR, YZ, bji, KM, Nq, es, bF, amS, cFS, eB, gZ, aS, DQ, bC, i, lC, hv); #c1(SLC3A2) c2(NP_001013269) c3(4F2 cell-surface antigen heavy chain isoform f) c4( ) c5(25450, 25451, 25452, 25453, 25454, 25455, 25456, 25457, 25458, 25459, 25460, 25461, 25462, 25463, 25464) c6( ) c7(bM, T, Ap, ea, fd, bgs, ame, ci, Qp, aY, bgM, x, bv, boB, K, bU, hu, pk, aDU, lZ, xQ, Pf, aAg, aW, az, apn, cFU, Au, eE, ou, y, iA, iT, go, vA, mx, aep, cj, bAD, k, m, R, hh, BV, dD, akc, QO, Or, aw, yG, ht, n, oY, fr, nA, Tl, cd, ai, tD, Oe, boz, b, cq, pe, xc, cc, ep, h, jy, eV, hn, aXd, ey, Nr, OV, yi, mR, beo, A, Cu, zb, cFT, lb, Ad, fe, po, Og, yq, CQ, aYo, aKv, bfW, Wt, op, asE, kT, dB, lf, bm, SZ, fL, eo, dl, bD, Wl, bL, Ob, aZA, fM, eR, jx, eX, aA, cz, Wa, V, pd, q, zn, bjA, aT, kf, cy, qU, atw, aWs, rO, KU, ae, aoL, hM, amx, iU, oV, ev, r, akB, chy, aom, hf, t, Vk, ho, pb, ik, vk, ck, cT, cCl, cl, OW, c, nM, iQ, hc, dy, ij, bt, Ze, chP, vi, BD, bre, Ke, Pt, aD, iW, oA, asa, aHv, jh, eq, fu, hF, tK, mC, aoH, chE, F, fX, py, ds, qh, oj, hD, chX, vc, ji, fb, chN, nJ, iL, apA, yF, uQ, MG, pq, aqa, qZ, xs, Of, cr, eZ, aF, hp, app, jg, iV, iR, YZ, bji, KM, Nq, es, bF, amS, cFS, eB, gZ, aS, DO, bC, i, lC, hv); #c1(SLC3A2) c2(NP_002385) c3(4F2 cell-surface antigen heavy chain isoform c) c4(128030) c5(128031, 128032, 128033, 128034, 128035, 128036) c6( ) c7(bM, T, Ap, ea, fd, bgs, ame, ci, Qp, aY, bgM, x, bv, boB, K, bU, hu, pk, aDU, lZ, xQ, Pf, aAg, aW, az, apn, cFU, Au, eE, ou, y, iA, iT, go, vA, mx, aep, cj, bAD, k, m, R, hh, BV, dD, akc, QO, Or, aw, yG, ht, n, oY, fr, nA, Tl, cd, ai, tD, Oe, boz, b, cq, pe, xc, cc, ep, h, jy, eV, hn, aXd, ey, Nr, OV, yi, mR, beo, A, Cu, zb, cFT, lb, Ad, fe, po, Og, yq, CQ, aYo, aKv, bfW, Wt, op, asE, kT, dB, lf, bm, SZ, fL, eo, dl, bD, Wl, bL, Ob, aZA, fM, eR, jx, eX, aA, cz, Wa, V, pd, q, zn, bjA, aT, kf, cy, qU, atw, aWs, rO, KU, ae, aoL, hM, amx, iU, oV, ev, r, akB, chy, aom, hf, t, Vk, ho, pb, ik, vk, ck, cT, cCl, cl, OW, c, nM, iQ, hc, dy, ij, bt, Ze, chP, vi, BD, bre, Ke, Pt, aD, iW, oA, asa, aHv, jh, eq, fu, hF, tK, mC, aoH, chE, F, fx, py, ds, qh, oj, hD, chX, vc, ji, fb, chN, nJ, iL, apA, yF, uQ, MG, pq, aqa, qZ, xs, Of, cr, eZ, aF, hp, app, jg, iV, iR, YZ, bji, KM, Nq, es, bF, amS, cFS, eB, gZ, aS, DQ, bC, i, lC, hv); #c1(SLC40A1) c2(NP_055400) c3(solute carrier family 40 member 1) c4(180797, 180798, 180799, 180800, 180801) c5(180802, 180803, 180804, 180805, 180806, 180807, 180808, 180809, 180810, 180811, 180812, 180813, 180814, 180815, 180816, 180817, 180818, 180819, 180820, 180821, 180822, 180823, 180824, 180825, 180826, 180827, 180828, 180829, 180830, 180831, 180832, 180833, 180834, 180835, 180836) c6( ) c7(al, ex, n, aUX, bHO, dP, yw, oW, aT, yu, eV, aiJ, beo, fc, atl, hl, yy, aq, bxM, dr, bN, BU, rR, aUW, abX, aUU, ao); #c1(SLC40A1) c2(XP_005246562) c3(solute carrier family 40 member 1 isoform X1) c4(267230) c5(267231, 267232, 267233, 267234, 267235) c6( ) c7(al, ex, n, aUX, bHO, dP, yw, oW, aT, yu, eV, aiJ, beo, fc, atl, hl, yy, aq, bxM, dr, bN, BU, rR, aUW, abX, aUU, ao); #c1(SLC41A1) c2(XP_005245126) c3(solute carrier family 41 member 1 isoform X1) c4(266308) c5(266309, 266310, 266311, 266312, 266313, 266314) c6( ) c7(byf, vm, EP, an); #c1(SLC43A1) c2(NP_001185739) c3(large neutral amino acids transporter small subunit 3) c4(76158, 76159) c5( ) c6( ) c7(py, wu, jh, aeu, wm, ae, ad); #c1(SLC43A1) c2(XP_005274415) c3(large neutral amino acids transporter small subunit 3 isoform X1) c4( ) c5(285067, 285068, 285069, 285070, 285071, 285072, 285073, 285074, 285075, 285076) c6( ) c7(py, wu, jh, aeu, wm, ae, ad); #c1(SLC43A1) c2(XP_011543622) c3(large neutral amino acids transporter small subunit 3 isoform X2) c4( ) c5(342631, 342632, 342633, 342634, 342635, 342636) c6( ) c7(py, wu, jh, aeu, wm, ae, ad); #c1(SLC43A2) c2(NP_001271428) c3(large neutral amino acids transporter small subunit 4 isoform 3) c4(103953) c5(103954, 103955, 103956, 103957, 103958, 103959) c6( ) c7(T, cER, cFL, bO, ti, v, agd, py, aT, aXd, K, akr, he, beo, nO, aW, ff, en, ik, qa, eM, cFK, bC, wS); #c1(SLC43A2) c2(NP_689559) c3(large neutral amino acids transporter small subunit 4 isoform 2) c4(243104) c5(243105, 243106, 243107, 243108, 243109, 243110) c6( ) c7(T, cER, cFL, bO, ti, v, agd, py, aT, aXd, K, akr, he, beo, nO, aW, ff, en, ik, qa, eM, cFK, bC, wS); #c1(SLC43A2) c2(XP_011521948) c3(large neutral amino acids transporter small subunit 4 isoform X1) c4(316644) c5(316645, 316646, 316647, 316648, 316649, 316650) c6( ) c7(T, cER, cFL, bO, ti, v, agd, py, aT, aXd, K, akr, he, beo, nO, aW, ff, en, ik, qa, eM, cFK, bC, wS); #c1(SLC43A2) c2(XP_011521950) c3 (large neutral amino acids transporter small subunit 4 isoform X3) c4( ) c5(316651) c6( ) c7(T, cER, cFL, bO, ti, v, agd, py, aT, aXd, K, akr, he, beo, nO, aW, ff, en, ik, qa, eM, cFK, bC, wS); #c1(SLC43A3) c2(NP_001265135) c3(solute carrier family 43 member 3 isoform 2) c4(97830) c5(97831, 97832, 97833, 97834, 97835, 97836, 97837, 97838, 97839, 97840) c6( ) c7(ey); #c1(SLC43A3) c2(NP_054815) c3(solute carrier family 43 member 3 isoform 1) c4(178012, 178013, 178014, 178015, 178016, 178017, 178018, 178019, 178020, 178021, 178022, 178023) c5(178024, 178025, 178026, 178027, 178028, 178029, 178030, 178031, 178032, 178033, 178034, 178035, 178036, 178037, 178038, 178039, 178040, 178041, 178042, 178043, 178044, 178045, 178046, 178047, 178048, 178049, 178050, 178051, 178052, 178053, 178054, 178055, 178056, 178057, 178058, 178059, 178060, 178061, 178062, 178063, 178064, 178065, 178066, 178067, 178068, 178069, 178070, 178071, 178072, 178073, 178074, 178075, 178076, 178077, 178078, 178079, 178080, 178081, 178082, 178083, 178084, 178085, 178086, 178087, 178088, 178089, 178090, 178091, 178092, 178093, 178094, 178095, 178096, 178097, 178098, 178099, 178100, 178101, 178102) c6( ) c7(ey); #c1(SLC44A1) c2(NP_001273659) c3(choline transporter-like protein 1 isoform b) c4(105563, 105564) c5(105565, 105566, 105567, 105568, 105569, 105570, 105571, 105572, 105573, 105574, 105575, 105576) c6( ) c7(cm, ft, eA, U, bz); #c1(SLC44A1) c2(NP_536856) c3(choline transporter-like protein 1 isoform a) c4(231373) c5(231374, 231375, 231376, 231377, 231378, 231379, 231380, 231381) c6( ) c7(cm, ft, eA, U, bz); #c1(SLC44A1) c2(XP_005251912) c3(choline transporter-like protein 1 isoform X2) c4( ) c5(271283, 271284) c6( ) c7(cm, ft, eA, U, bz); #c1(SLC44A1) c2(XP_006717092) c3(choline transporter-like protein 1 isoform X4) c4(290712) c5(290713, 290714, 290715, 290716, 290717, 290718) c6( ) c7(cm, ft, eA, U, bz); #c1(SLC44A2) c2(NP_001138528) c3(choline transporter-like protein 2 isoform 2) c4(56964) c5(56965, 56966, 56967, 56968, 56969, 56970) c6( ) c7(aFA); #c1(SLC44A2) c2(NP_065161) c3(choline transporter-like protein 2 isoform 1) c4(204348) c5(204349, 204350, 204351, 204352, 204353, 204354) c6( ) c7(aFA); #c1(SLC44A2) c2(XP_005260054) c3(choline transporter-like protein 2 isoform X1) c4(276421) c5(276422, 276423, 276424, 276425, 276426, 276427) c6( ) c7(aFA); #c1(SLC44A4) c2(NP_001171515) c3(choline transporter-like protein 4 isoform 2) c4(70392, 70393) c5(70394, 70395, 70396, 70397, 70398, 70399, 70400, 70401, 70402, 70403, 70404, 70405, 70406, 70407, 70408, 70409, 70410, 70411, 70412, 70413, 70414, 70415, 70416, 70417, 70418, 70419, 70420) c6( ) c7(V, A, gv, qj, eZ); #c1(SLC44A4) c2(NP_001171516) c3(choline transporter-like protein 4 isoform 3) c4(70421, 70422) c5(70423, 70424, 70425, 70426, 70427, 70428, 70429, 70430, 70431, 70432, 70433, 70434, 70435, 70436, 70437, 70438, 70439, 70440, 70441, 70442, 70443, 70444, 70445, 70446, 70447, 70448) c6( ) c7(V, A, gv, qj, eZ); #c1(SLC44A4) c2(NP_079533) c3(choline transporter-like protein 4 isoform 1) c4(218645, 218646, 218647, 218648) c5(218649, 218650, 218651, 218652, 218653, 218654, 218655, 218656, 218657, 218658, 218659, 218660, 218661, 218662, 218663, 218664, 218665, 218666, 218667, 218668, 218669, 218670, 218671, 218672, 218673, 218674, 218675, 218676, 218677, 218678, 218679, 218680, 218681, 218682, 218683, 218684, 218685, 218686, 218687, 218688, 218689, 218690, 218691, 218692, 218693, 218694, 218695, 218696, 218697, 218698, 218699, 218700, 218701, 218702, 218703, 218704) c6( ) c7(V, A, gv, qj, eZ); #c1(SLC44A5) c2(XP_011539285) c3(choline transporter-like protein 5 isoform X3) c4(337354) c5( ) c6( ) c7(e, fq, af); #c1(SLC44A5) c2(XP_011539287) c3(choline transporter-like protein 5 isoform X4) c4(337355) c5(337356, 337357, 337358, 337359, 337360, 337361) c6( ) c7(e, fq, af); #c1(SLC45A2) c2(NP_001012527) c3(membrane-associated transporter protein isoform b) c4(25052) c5(25053, 25054, 25055, 25056, 25057, 25058) c6( ) c7(gV, T, bM, n, cFW, q, ad, byF, afQ, aT, ae, ahU, cFV, cp, hG, aW, Y, hC, ff, r, aid, en, bz, ck, sS, atU, Dy, hk, lB); #c1(SLC45A2) c2(NP_001284346) c3(membrane-associated transporter protein isoform c) c4(111720) c5(111721, 111722, 111723, 111724, 111725, 111726, 111727, 111728, 111729, 111730, 111731, 111732) c6( ) c7(gV, T, bM, n, cFW, q, ad, byF, afQ, aT, ae, ahU, cFV, cp, hG, aW, Y, hC, ff, r, aid, en, bz, ck, sS, atU, Dy, hk, lB); #c1(SLC45A2) c2(NP_057264) c3(membrane-associated transporter protein isoform a) c4(189760, 189761, 189762) c5(189763, 189764, 189765, 189766, 189767, 189768) c6( ) c7(gV, T, bM, n, cFW, q, ad, byF, afQ, aT, ae, ahU, cFV, cp, hG, aW, Y, hC, ff, r, aid, en, bz, ck, sS, atU, Dy, hk, lB); #c1(SLC45A2) c2(XP_011512353) c3(membrane-associated transporter protein isoform X1) c4(303050) c5(303051, 303052, 303053, 303054, 303055, 303056, 303057, 303058, 303059, 303060, 303061, 303062) c6( ) c7(gV, T, bM, n, cFW, q, ad, byF, afQ, aT, ae, ahU, cFV, cp, hG, aW, Y, hC, ff, r, aid, en, bz, ck, sS, atU, Dy, hk, lB); #c1(SLC45A3) c2(NP_149093) c3(solute carrier family 45 member 3) c4(227103) c5(227104, 227105, 227106, 227107, 227108, 227109) c6( ) c7(bM, T, ci, an, ae, ad); #c1(SLC45A4) c2(NP_001073900) c3(solute carrier family 45 member 4 isoform 2) c4(36362) c5(36363, 36364, 36365, 36366, 36367, 36368, 36369, 36370) c6( ) c7(he, bK); #c1(SLC45A4) c2(NP_001273575) c3(solute carrier family 45 member 4 isoform 1) c4(105331, 105332) c5(105333, 105334, 105335, 105336, 105337, 105338, 105339, 105340, 105341, 105342, 105343, 105344) c6( ) c7(he, bK); #c1(SLC45A4) c2(NP_001273577) c3(solute carrier family 45 member 4 isoform 3) c4(105345) c5(105346, 105347, 105348, 105349, 105350, 105351) c6( ) c7(he, bK); #c1(SLC46A1) c2(NP_001229295) c3(proton-coupled folate transporter isoform 2) c4(84165) c5(84166, 84167, 84168, 84169, 84170, 84171) c6( ) c7(ng, fU, T, mO, ck, dA, yw, bwm, ib, eM, wE, oW, yy, oX, ao); #c1(SLC46A1) c2(NP_542400) c3(proton-coupled folate transporter isoform 1) c4(232344) c5(232345, 232346, 232347, 232348, 232349, 232350) c6( ) c7(ng, fU, T, mO, ck, dA, yw, bwm, ib, eM, wE, oW, yy, oX, ao); #c1(SLC46A2) c2(NP_149040) c3(thymic stromal cotransporter homolog) c4(226896) c5(226897, 226898, 226899, 226900, 226901, 226902) c6( ) c7(if, qN); #c1(SLC47A1) c2(NP_060712) c3(multidrug and toxin extrusion protein 1) c4(197342, 197343, 197344, 197345, 197346, 197347) c5(197348, 197349, 197350, 197351, 197352, 197353, 197354, 197355, 197356, 197357, 197358, 197359, 197360, 197361, 197362, 197363, 197364, 197365, 197366, 197367, 197368, 197369, 197370, 197371, 197372, 197373, 197374, 197375, 197376, 197377, 197378, 197379, 197380, 197381, 197382, 197383, 197384, 197385, 197386, 197387) c6( ) c7(bL, aj, dy, J, SE); #c1(SLC48A1) c2(NP_060312) c3(heme transporter HRG1) c4(195323, 195324) c5(195325, 195326, 195327, 195328, 195329, 195330, 195331, 195332, 195333, 195334, 195335, 195336, 195337, 195338, 195339, 195340, 195341, 195342, 195343, 195344, 195345, 195346, 195347, 195348, 195349, 195350, 195351, 195352, 195353, 195354, 195355, 195356, 195357) c6( ) c7(aW, T, aT); #c1(SLC48A1) c2(XP_005269073) c3(heme transporter HRG1 isoform X2) c4(282292) c5( ) c6( ) c7(aW, T, aT); #c1(SLC48A1) c2(XP_011536860) c3(heme transporter HRG1 isoform X1) c4(334557, 334558) c5(334559) c6( ) c7(aW, T, aT); #c1(SLC4A1) c2(NP_000333) c3(band 3 anion transport protein) c4(7362) c5(7363, 7364, 7365, 7366, 7367, 7368) c6( ) c7(NU, T, eR, cFX, fR, zs, oe, aA, cz, oY, vm, cGc, aY, cGb, eh, yn, cGa, Tl, TK, oW, dV, cGd, ajT, aZf, Fm, vj, aF, S, g, aot, qs, bqC, eY, BU, VJ, cGe, cFY, fP, oh, pw, Cl, cfR, wS, cFZ); #c1(SLC4A10) c2(NP_001171486) c3(sodium-driven chloride bicarbonate exchanger isoform 1) c4(70293, 70294) c5(70295, 70296, 70297, 70298, 70299, 70300, 70301, 70302, 70303, 70304, 70305) c6( ) c7(acf, aca, pZ, ha, jx, bS, ct, nA, Z, aNX); #c1(SLC4A10) c2(NP_001171487) c3(sodium-driven chloride bicarbonate exchanger isoform 3) c4(70306) c5(70307, 70308, 70309, 70310, 70311) c6( ) c7(acf, aca, pZ, ha, jx, bS, ct, nA, Z, aNX); #c1(SLC4A10) c2(NP_071341) c3(sodium-driven chloride bicarbonate exchanger isoform 2) c4(211101) c5(211102, 211103, 211104, 211105, 211106) c6( ) c7(acf, aca, pZ, ha, jx, bS, ct, nA, Z, aNX); #c1(SLC4A10) c2(XP_005246752) c3(sodium-driven chloride bicarbonate exchanger isoform X9) c4( ) c5(267413) c6( ) c7(acf, aca, pZ, ha, jx, bS, ct, nA, Z, aNX); #c1(SLC4A10) c2(XP_006712706) c3(sodium-driven chloride bicarbonate exchanger isoform X6) c4( ) c5(286811) c6( ) c7(acf, aca, pZ, ha, jx, bS, ct, nA, Z, aNX); #c1(SLC4A10) c2(XP_011509810) c3(sodium-driven chloride bicarbonate exchanger isoform X1) c4(300375) c5(300376, 300377) c6( ) c7(acf, aca, pZ, ha, jx, bS, ct, nA, Z, aNX); #c1(SLC4A10) c2(XP_011509811) c3(sodium-driven chloride bicarbonate exchanger isoform X2) c4(300378) c5(300379, 300380, 300381, 300382, 300383, 300384) c6( ) c7(acf, aca, pZ, ha, jx, bS, ct, nA, Z, aNX); #c1(SLC4A10) c2(XP_011509813) c3(sodium-driven chloride bicarbonate exchanger isoform X4) c4(300385) c5(300386, 300387, 300388, 300389, 300390, 300391, 300392) c6( ) c7(acf, aca, pZ, ha, jx, bS, ct, nA, Z, aNX); #c1(SLC4A10) c2(XP_011509814) c3(sodium-driven chloride bicarbonate exchanger isoform X5) c4(300393) c5(300394, 300395, 300396, 300397, 300398, 300399) c6( ) c7(acf, aca, pZ, ha, jx, bS, ct, nA, Z, aNX); #c1(SLC4A10) c2(XP_011509817) c3(sodium-driven chloride bicarbonate exchanger isoform X10) c4( ) c5(300400, 300401, 300402, 300403, 300404) c6( ) c7(acf, aca, pZ, ha, jx, bS, ct, nA, Z, aNX); #c1(SLC4A11) c2(NP_001167560) c3(sodium bicarbonate transporter-like protein fl isoform 3) c4(69245, 69246) c5(69247, 69248, 69249, 69250, 69251, 69252, 69253, 69254, 69255, 69256, 69257, 69258) c6( ) c7(cGg, Lu, alH, aOu, cGh, aW, bmD, mG, la, cGf, aT, ab, cGj, all, cGi); #c1(SLC4A11) c2(NP_001167561) c3(sodium bicarbonate transporter-like protein 11 isoform 1) c4(69259) c5(69260, 69261, 69262, 69263, 69264, 69265) c6( ) c7(cGg, Lu, alH, aOu, cGh, aW, bmD, mG, la, cGf, aT, ab, cGj, all, cGi); #c1(SLC4A11) c2(NP_114423) c3(sodium bicarbonate transporter-like protein 11 isoform 2) c4(222692) c5(222693, 222694, 222695, 222696, 222697, 222698) c6( ) c7(cGg, Lu, alH, aOu, cGh, aW, bmD, mG, la, cGf, aT, ab, cGj, all, cGi); #c1(SLC4A1AP) c2(NP_060628) c3(kanadaptin) c4(196898) c5(196899, 196900, 196901, 196902, 196903, 196904) c6( ) c7(af); #c1(SLC4A2) c2(NP_001186622) c3(anion exchange protein 2 isoform 2) c4(77988) c5(77989, 77990, 77991, 77992, 77993, 77994) c6( ) c7(jz, ZZ, bE, vj, hl, aA, bB, at, dL, aF, bc); #c1(SLC4A2) c2(NP_001186623) c3(anion exchange protein 2 isoform 3) c4(77995, 77996) c5(77997, 77998, 77999, 78000, 78001, 78002) c6( ) c7(jz, ZZ, bE, vj, hl, aA, bB, at, dL, aF, bc); #c1(SLC4A2) c2(XP_006716157) c3(anion exchange protein 2 isoform X2) c4( ) c5(290106, 290107, 290108, 290109, 290110, 290111, 290112, 290113, 290114, 290115, 290116, 290117, 290118, 290119, 290120, 290121, 290122, 290123, 290124, 290125, 290126, 290127, 290128, 290129, 290130, 290131, 290132, 290133, 290134, 290135) c6( ) c7(jz, ZZ, bE, vj, hl, aA, bB, at, dL, aF, bc); #c1(SLC4A2) c2(XP_011514799) c3(anion exchange protein 2 isoform X1) c4(307447, 307448, 307449, 307450, 307451, 307452) c5(307453, 307454, 307455, 307456, 307457, 307458, 307459, 307460, 307461, 307462, 307463, 307464, 307465, 307466, 307467, 307468) c6( ) c7(jz, ZZ, bE, vj, hl, aA, bB, at, dL, aF, bc); #c1(SLC4A3) c2(NP_005061) c3(anion exchange protein 3 isoform 1) c4(152514) c5(152515, 152516, 152517, 152518, 152519, 152520, 152521, 152522, 152523, 152524, 152525, 152526, 152527, 152528, 152529) c6( ) c7(gZ, nw, ha, aQw); #c1(SLC4A3) c2(NP_963868) c3(anion exchange protein 3 isoform 2) c4(262479) c5(262480) c6( ) c7(gZ, nw, ha, aQw); #c1(SLC4A3) c2(XP_005246846) c3(anion exchange protein 3 isoform X1) c4(267476) c5(267477, 267478, 267479, 267480, 267481, 267482, 267483, 267484, 267485, 267486) c6( ) c7(gZ, nw, ha, aQw); #c1(SLC4A3) c2(XP_005246847) c3(anion exchange protein 3 isoform X4) c4(267487) c5(267488, 267489, 267490, 267491, 267492, 267493) c6( ) c7(gZ, nw, ha, aQw); #c1(SLC4A3) c2(XP_011509969) c3(anion exchange protein 3 isoform X5) c4( ) c5(300593, 300594, 300595, 300596, 300597) c6( ) c7(gZ, nw, ha, aQw); #c1(SLC4A4) c2(NP_001091954) c3 (electrogenic sodium bicarbonate cotransporter 1 isoform 1) c4(39003, 39004) c5(39005, 39006, 39007, 39008, 39009, 39010) c6( ) c7(cGl, lR, ajT, DD, zs, cz, cGk, fr, nA, dw, yj, ao); #c1(SLC4A4) c2(NP_001128214) c3(electrogenic sodium bicarbonate cotransporter 1 isoform 3) c4(49713) c5(49714, 49715, 49716, 49717, 49718, 49719, 49720, 49721, 49722, 49723, 49724, 49725) c6( ) c7(cGl, lR, ajT, DD, zs, cz, cGk, fr, nA, dw, yj, ao); #c1(SLC4A4) c2(NP_003750) c3(electrogenic sodium bicarbonate cotransporter 1 isoform 2) c4(140617) c5(140618, 140619, 140620, 140621, 140622, 140623) c6( ) c7(cGl, lR, ajT, DD, zs, cz, cGk, fr, nA, dw, yj, ao); #c1(SLC4A5) c2(NP_067019) c3(electrogenic sodium bicarbonate cotransporter 4 isoform a) c4(208407, 208408, 208409) c5(208410, 208411, 208412, 208413, 208414, 208415, 208416, 208417, 208418, 208419, 208420, 208421, 208422, 208423, 208424, 208425, 208426, 208427, 208428, 208429, 208430, 208431, 208432, 208433, 208434, 208435, 208436, 208437, 208438, 208439, 208440, 208441, 208442, 208443, 208444, 208445, 208446, 208447) c6( ) c7(v, oe, cz); #c1(SLC4A5) c2(NP_597812) c3(electrogenic sodium bicarbonate cotransporter 4 isoform c) c4(235163, 235164, 235165, 235166, 235167, 235168) c5(235169, 235170, 235171, 235172, 235173, 235174, 235175, 235176, 235177, 235178, 235179, 235180, 235181, 235182, 235183, 235184, 235185, 235186, 235187, 235188, 235189, 235190, 235191, 235192, 235193, 235194, 235195, 235196) c6( ) c7(v, oe, cz); #c1(SLC4A7) c2(NP_001245308) c3(sodium bicarbonate cotransporter 3 isoform b) c4(93071, 93072) c5(93073, 93074, 93075, 93076, 93077, 93078, 93079, 93080, 93081, 93082, 93083, 93084, 93085, 93086, 93087, 93088, 93089, 93090, 93091, 93092) c6( ) c7(FK, T, eM, Pt, aW, PE, aT, lB, cE, ao); #c1(SLC4A7) c2(NP_001245309) c3(sodium bicarbonate cotransporter 3 isoform c) c4(93093) c5(93094, 93095, 93096, 93097, 93098, 93099, 93100, 93101, 93102) c6( ) c7(FK, T, eM, Pt, aW, PE, aT, lB, cE, ao); #c1(SLC4A7) c2(NP_003606) c3(sodium bicarbonate cotransporter 3 isoform a) c4(139536) c5(139537, 139538, 139539, 139540, 139541, 139542) c6( ) c7(FK, T, eM, Pt, aW, PE, aT, lB, cE, ao); #c1(SLC4A7) c2(XP_005265658) c3(sodium bicarbonate cotransporter 3 isoform X15) c4(279654) c5(279655, 279656, 279657, 279658, 279659, 279660, 279661, 279662, 279663, 279664, 279665, 279666, 279667, 279668) c6( ) c7(FK, T, eM, Pt, aW, PE, aT, lB, cE, ao); #c1(SLC4A7) c2(XP_011532557) c3(sodium bicarbonate cotransporter 3 isoform X1) c4(328652) c5(328653, 328654, 328655, 328656, 328657, 328658) c6( ) c7(FK, T, eM, Pt, aW, PE, aT, lB, cE, ao); #c1(SLC4A7) c2(XP_011532559) c3(sodium bicarbonate cotransporter 3 isoform X4) c4(328659, 328660, 328661) c5(328662, 328663, 328664, 328665, 328666, 328667) c6( ) c7(FK, T, eM, Pt, aW, PE, aT, lB, cE, ao); #c1(SLC4A7) c2(XP_011532562) c3(sodium bicarbonate cotransporter 3 isoform X7) c4(328668) c5(328669, 328670, 328671, 328672, 328673, 328674, 328675, 328676) c6( ) c7(FK, T, eM, Pt, aW, PE, aT, lB, cE, ao); #c1(SLC4A7) c2(XP_011532564) c3(sodium bicarbonate cotransporter 3 isoform X9) c4(328677) c5(328678, 328679, 328680, 328681, 328682, 328683) c6( ) c7(FK, T, eM, Pt, aW, PE, aT, lB, cE, ao); #c1(SLC4A9) c2(NP_001245355) c3 (anion exchange protein 4 isoform 3) c4(93228) c5(93229, 93230, 93231, 93232, 93233) c6( ) c7(oV, ft, eM); #c1(SLC4A9) c2(NP_001245356) c3(anion exchange protein 4 isoform 4) c4(93234) c5(93235, 93236, 93237, 93238, 93239) c6( ) c7(oV, ft, eM); #c1(SLC4A9) c2(NP_001245357) c3(anion exchange protein 4 isoform 1) c4(93240) c5( ) c6( ) c7(oV, ft, eM); #c1(SLC4A9) c2(XP_005268578) c3(anion exchange protein 4 isoform X3) c4(281929) c5(281930, 281931, 281932, 281933, 281934, 281935) c6( ) c7(oV, ft, eM); #c1(SLC4A9) c2(XP_006714862) c3(anion exchange protein 4 isoform X12) c4( ) c5(288271) c6( ) c7(oV, ft, eM); #c1(SLC4A9) c2(XP_011535988) c3(anion exchange protein 4 isoform X1) c4( ) c5(333255, 333256, 333257, 333258, 333259, 333260, 333261, 333262, 333263, 333264) c6( ) c7(oV, ft, eM); #c1(SLC4A9) c2(XP_011535992) c3(anion exchange protein 4 isoform X6) c4( ) c5(333265) c6( ) c7(oV, ft, eM); #c1(SLC50A1) c2(NP_001116309) c3(sugar transporter SWEET1 isoform b) c4(43746) c5(43747, 43748, 43749, 43750, 43751, 43752) c6( ) c7(Ni, n, ci, cGm, bD, eW); #c1(SLC50A1) c2(NP_001116311) c3(sugar transporter SWEET1 isoform c) c4(43753) c5(43754, 43755, 43756, 43757, 43758, 43759) c6( ) c7(Ni, n, ci, cGm, bD, eW); #c1(SLC50A1) c2(NP_001274519) c3(sugar transporter SWEET1 isoform h) c4(106602) c5(106603, 106604, 106605, 106606, 106607, 106608) c6( ) c7(Ni, n, ci, cGm, bD, eW); #c1(SLC50A1) c2(NP_061333) c3(sugar transporter SWEET1 isoform a) c4(200102) c5(200103, 200104, 200105, 200106, 200107, 200108) c6( ) c7(Ni, n, ci, cGm, bD, eW); #c1(SLC51A) c2(NP_689885) c3(organic solute transporter subunit alpha) c4(244253) c5(244254, 244255, 244256, 244257, 244258, 244259) c6( ) c7(hJ, bc); #c1(SLC51B) c2(NP_849190) c3(organic solute transporter subunit beta) c4(254466) c5(254467, 254468, 254469, 254470, 254471, 254472) c6( ) c7(v, l, bc, u, hJ); #c1(SLC52A1) c2(NP_060456) c3(solute carrier family 52, riboflavin transporter, member 1 precursor) c4(196032, 196033) c5(196034, 196035, 196036, 196037, 196038, 196039, 196040, 196041, 196042, 196043, 196044, 196045, 196046, 196047, 196048, 196049) c6( ) c7(lD, bM, T, Mb, fG, aw, ez, bB, aj, ci, qN, V, cd, ad, J, BY, dP, ke, ai, eA, av, aE, ZX, dv, lC, aT, h, cy, ae, aW, czf, ff, cr, fe, gl, en, g, iV, t, zV, LB, y, xZ, hk, at, Km, m, fD, ra, zB, cE, ao, aZN); #c1(SLC52A2) c2(NP_001240745) c3(solute carrier family 52, riboflavin transporter, member 2) c4(88722, 88723) c5(88724, 88725, 88726, 88727, 88728, 88729, 88730, 88731, 88732, 88733, 88734, 88735, 88736, 88737, 88738, 88739, 88740, 88741, 88742, 88743, 88744, 88745, 88746, 88747, 88748, 88749, 88750, 88751, 88752, 88753, 88754, 88755) c6( ) c7(bM, T, aC, fG, aA, bB, n, wV, MC, j, ci, C, fu, fr, rc, ad, ady, bv, av, jz, F, aE, vJ, aT, ae, af, cp, aa, mH, OV, d, B, nO, aW, Y, auM, az, eZ, lo, aF, cGn, cl, ik, bz, bdu, LB, ck, EW, es, iA, bpY, bE, acS, uR, hL, cGo, at, aiS, m, fD, ckG, y, bt, cE, X); #c1(SLC52A2) c2(XP_011515602) c3(solute carrier family 52, riboflavin transporter, member 2 isoform X2) c4( ) c5(308449, 308450, 308451, 308452, 308453, 308454) c6( ) c7(bM, T, aC, fG, aA, bB, n, wV, MC, j, ci, C, fu, fr, rc, ad, ady, bv, av, jz, F, aE, vJ, aT, ae, af, cp, aa, mH, OV, d, B, nO, aW, Y, auM, az, eZ, lo, aF, cGn, cl, ik, bz, bdu, LB, ck, EW, es, iA, bpY, bE, acS, uR, hL, cGo, at, aiS, m, fD, ckG, y, bt, cE, X); #c1(SLC52A3) c2(XP_011527450) c3(solute carrier family 52, riboflavin transporter, member 3 isoform X1) c4(322214, 322215) c5(322216, 322217, 322218, 322219, 322220, 322221, 322222, 322223, 322224, 322225, 322226, 322227, 322228) c6( ) c7(biq, c, pf, Ol, ff, ht, gX, cGq, cGn, hu, en, it, cn, cGp); #c1(SLC5A1) c2(NP_000334) c3(sodium/glucose cotransporter 1 isoform 1) c4(7369, 7370) c5(7371, 7372, 7373, 7374, 7375, 7376, 7377, 7378, 7379, 7380, 7381, 7382) c6( ) c7(bL, T, A, s, vj, lz, cFt, arC, jN, lZ, gZ, if, j, K, g, lm, cgu); #c1(SLC5A11) c2(NP_001245340) c3(sodium/myo-inositol cotransporter 2 isoform 2) c4(93138) c5(93139, 93140, 93141, 93142, 93143, 93144) c6( ) c7(lp, aj, kl, eZ); #c1(SLC5A11) c2(NP_001245341) c3(sodium/myo-inositol cotransporter 2 isoform 3) c4(93145) c5(93146, 93147, 93148, 93149, 93150, 93151) c6( ) c7(lp, aj, kl, eZ); #c1(SLC5A11) c2(NP_001245342) c3(sodium/myo-inositol cotransporter 2 isoform 4) c4( ) c5(93152, 93153, 93154, 93155, 93156, 93157, 93158, 93159) c6( ) c7(lp, aj, kl, eZ); #c1(SLC5A11) c2(NP_001245343) c3(sodium/myo-inositol cotransporter 2 isoform 5) c4( ) c5(93160, 93161, 93162, 93163, 93164) c6( ) c7(lp, aj, kl, eZ); #c1(SLC5A11) c2(NP_443176) c3(sodium/myo-inositol cotransporter 2 isoform 1) c4(229718) c5(229719, 229720, 229721, 229722, 229723, 229724, 229725, 229726) c6( ) c7(lp, aj, kl, eZ); #c1(SLC5A11) c2(XP_011544025) c3(sodium/myo-inositol cotransporter 2 isoform X2) c4(343194) c5( ) c6( ) c7(lp, aj, kl, eZ); #c1(SLC5A11) c2(XP_011544028) c3(sodium/myo-inositol cotransporter 2 isoform X5) c4(343195) c5(343196) c6( ) c7(lp, aj, kl, eZ); #c1(SLC5A12) c2(NP_848593) c3(sodium-coupled monocarboxylate transporter 2) c4(253927) c5(253928, 253929, 253930, 253931, 253932, 253933, 253934) c6( ) c7(bM, ac); #c1(SLC5A12) c2(XP_006718220) c3(sodium-coupled monocarboxylate transporter 2 isoform X5) c4(291583) c5(291584, 291585, 291586, 291587, 291588, 291589) c6( ) c7(bM, ac); #c1(SLC5A12) c2(XP_011518223) c3(sodium-coupled monocarboxylate transporter 2 isoform X4) c4(312261) c5(312262, 312263, 312264, 312265, 312266) c6( ) c7(bM, ac); #c1(SLC5A2) c2(NP_003032) c3(sodium/glucose cotransporter 2) c4(134573) c5(134574, 134575, 134576, 134577, 134578, 134579) c6( ) c7(bL, kQ, cFt, bo, lZ, cz, jy, aj, dG, K, uL, J, blX); #c1(SLC5A2) c2(XP_006721136) c3(sodium/glucose cotransporter 2 isoform X2) c4(293868) c5(293869, 293870, 293871, 293872, 293873, 293874) c6( ) c7(bL, kQ, cFt, bo, lZ, cz, jy, aj, dG, K, uL, J, blX); #c1(SLC5A3) c2(NP_008864) c3(sodium/myo-inositol cotransporter) c4(169311) c5(169312, 169313, 169314, 169315, 169316, 169317) c6( ) c7(kl, vA, aw, aj, j, K, bK, fr, op); #c1(SLC5A4) c2(NP_055042) c3(low affinity sodium-glucose cotransporter) c4(178507) c5(178508, 178509, 178510, 178511, 178512, 178513) c6( ) c7(bL, T, kQ, cFt, bo, lZ, jy, aj, dG, uL, blX); #c1(SLC5A5) c2(NP_000444) c3(sodium/iodide cotransporter) c4(9057) c5(9058, 9059, 9060, 9061, 9062, 9063) c6( ) c7(T, aC, nl, wu, aw, aA, bB, hz, fd, Fb, ci, fu, q, ad, er, eA, Vp, hd, no, xy, uT, ib, nv, Db, aE, pX, jz, RN, aT, cU, hD, Fx, AN, wm, nJ, ae, af, yF, afu, jf, d, gP, s, aW, Y, ff, r, aq, lo, aF, CQ, en, bu, iR, t, zE, bz, cm, qJ, es, bF, aZT, nM, eM, nB, bE, eD, hR, m, gT, B, cGr); #c1(SLC5A6) c2(XP_0067 (2193) c3(sodium-dependent multivitamin transporter isoform X1) c4(286512) c5(286513, 286514, 286515, 286516, 286517, 286518, 286519, 286520, 286521, 286522, 286523, 286524, 286525, 286526, 286527, 286528) c6( ) c7(aW, ae, ad, aT); #c1(SLC5A6) c2(XP_006712194) c3(sodium-dependent multivitamin transporter isoform X5) c4(286529, 286530, 286531, 286532, 286533, 286534, 286535, 286536, 286537) c5(286538, 286539, 286540, 286541, 286542, 286543, 286544, 286545, 286546, 286547, 286548, 286549, 286550, 286551, 286552, 286553, 286554, 286555, 286556, 286557, 286558, 286559, 286560, 286561, 286562, 286563, 286564, 286565, 286566, 286567, 286568, 286569, 286570, 286571, 286572, 286573, 286574, 286575, 286576, 286577, 286578, 286579, 286580, 286581, 286582, 286583, 286584, 286585) c6( ) c7(aW, ae, ad, aT); #c1(SLC5A7) c2(NP_001291934) c3(high affinity choline transporter 1 isoform a) c4(116517) c5(116518, 116519, 116520, 116521, 116522, 116523, 116524, 116525, 116526, 116527, 116528) c6( ) c7(cA, agl, cGs, gf, K, aiO); #c1(SLC5A7) c2(NP_001291935) c3(high affinity choline transporter 1 isoform b) c4(116529) c5(116530, 116531, 116532, 116533, 116534) c6( ) c7(cA, agl, cGs, gf, K, ail); #c1(SLC5A8) c2(NP_666018) c3(sodium-coupled monocarboxylate transporter 1) c4(241999) c5(242000, 242001, 242002, 242003, 242004, 242005) c6( ) c7(T, aC, aA, bB, ci, Fc, q, ad, eA, eJ, av, nH, hd, aE, Db, aT, Fx, ae, s, aW, ff, r, aF, en, as, iV, bz, ck, nM, bE, bt, X); #c1(SLC6A1) c2(NP_003033) c3(sodium- and chloride-dependent GABA transporter 1) c4(134580, 134581, 134582) c5(134583, 134584, 134585, 134586, 134587, 134588, 134589, 134590, 134591, 134592, 134593, 134594, 134595, 134596, 134597, 134598, 134599, 134600) c6( ) c7(il, cGt, QR, c, Gp, ha, Z, eu, ek, hq); #c1(SLC6A1l) c2(NP_055044) c3(sodium- and chloride-dependent GABA transporter 3) c4(178521, 178522) c5(178523, 178524, 178525, 178526, 178527, 178528) c6( ) c7(bhB, Y, vj, ha, lG, y, cv); #c1(SLC6A12) c2(XP_005253803) c3(sodium- and chloride-dependent betaine transporter isoform X1) c4(272668) c5(272669, 272670, 272671, 272672, 272673, 272674, 272675, 272676, 272677, 272678, 272679, 272680, 272681, 272682, 272683, 272684, 272685, 272686, 272687, 272688, 272689, 272690) c6( ) c7(bM, vj, ha, jx, v, K, as, af); #c1(SLC6A12) c2(XP_005253804) c3(sodium- and chloride-dependent betaine transporter isoform X2) c4(272691) c5( ) c6( ) c7(bM, vj, ha, jx, v, K, as, af); #c1(SLC6A12) c2(XP_011519313) c3(sodium- and chloride-dependent betaine transporter isoform X5) c4( ) c5(313967, 313968, 313969, 313970, 313971, 313972) c6( ) c7(bM, vj, ha, jx, v, K, as, af); #c1(SLC6A13) c2(NP_001177926) c3(sodium- and chloride-dependent GABA transporter 2 isoform 2) c4(73187) c5(73188, 73189, 73190, 73191, 73192, 73193) c6( ) c7(he, vj, ha, eR); #c1(SLC6A13) c2(NP_001230321) c3(sodium- and chloride-dependent GABA transporter 2 isoform 3) c4(86230) c5(86231, 86232, 86233, 86234, 86235, 86236) c6( ) c7(he, vj, ha, eR); #c1(SLC6A13) c2(NP_057699) c3(sodium- and chloride-dependent GABA transporter 2 isoform 1) c4( ) c5(192426, 192427, 192428, 192429, 192430, 192431, 192432, 192433, 192434, 192435, 192436) c6( ) c7(he, vj, ha, eR); #c1(SLC6A13) c2(XP_005253806) c3(sodium- and chloride-dependent GABA transporter 2 isoform X1) c4(272692, 272693) c5(272694) c6( ) c7(he, vj, ha, eR); #c1(SLCGA14) c2(NP_009162) c3(sodium- and chloride-dependent neutral and basic amino acid transporter B(O+)) c4(171199) c5(171200, 171201, 171202, 171203, 171204, 171205) c6( ) c7(LA, iV, cGu, aW, aZs, aT, fe, v, cFn, bC, qN, wS, aa); #c1(SLC6A15) c2(NP_060527) c3(sodium-dependent neutral amino acid transporter B(O)AT2 isoform 2) c4(196389) c5(196390, 196391, 196392, 196393, 196394, 196395, 196396, 196397, 196398, 196399, 196400, 196401) c6( ) c7(bO, bS, v, K, ct, Z, he); #c1(SLC6A15) c2(XP_011536825) c3(sodium-dependent neutral amino acid transporter B(O)AT2 isoform X1) c4(334512, 334513, 334514) c5(334515, 334516, 334517, 334518, 334519, 334520, 334521, 334522, 334523, 334524, 334525, 334526, 334527) c6( ) c7(bO, bS, v, K, ct, Z, he); #c1(SLC6A15) c2(XP_011536826) c3(sodium-dependent neutral amino acid transporter B(O)AT2 isoform X2) c4( ) c5(334528, 334529, 334530, 334531, 334532) c6( ) c7(b, bS, v, K, ct, Z, he); #c1(SLC6A15) c2(XP_011536827) c3(sodium-dependent neutral amino acid transporter B(O)AT2 isoform X3) c4(334533) c5( ) c6( ) c7(bO, bS, v, K, ct, Z, he); #c1(SLC6A18) c2(NP_872438) c3(sodium-dependent neutral amino acid transporter B(O)AT3) c4(256376, 256377) c5(256378, 256379, 256380, 256381, 256382, 256383, 256384, 256385, 256386, 256387, 256388, 256389) c6( ) c7(cFK, dL, cFL, cz); #c1(SLC6A19) c2(NP_001003841) c3(sodium-dependent neutral amino acid transporter B(O)AT1) c4(20227) c5(20228, 20229, 20230, 20231, 20232, 20233) c6( ) c7(cd, cFK, cFL, wS, cz); #c1(SLC6A2) c2(NP_001165972) c3(sodium-dependent noradrenaline transporter isoform 2) c4(68053) c5(68054, 68055, 68056, 68057, 68058, 68059, 68060, 68061, 68062, 68063) c6( ) c7(azs, T, bO, bB, ci, lF, aE, iX, pX, K, eL, Jl, aW, g, cm, aiO, aqr, cbM, mx, QF, uJ, wJ, To, ww, ct, aBl, cd, ft, amJ, awb, dL, he, Jj, aq, Jh, hq, ok, cA, dB, uz, jd, B, lI, oe, DV, aA, cz, v, j, q, aT, bK, jj, ae, bkl, ak, avV, blD, s, yL, r, en, iK, aFA, bdn, cT, AH, yH, Fl, awa, VB, blu, gx, pY, iW, OR, bS, ad, av, wv, afm, Od, qE, tb, vj, ff, aF, aTR, Z, bz, apM, Ew, bE, eB, ra, hv); #c1(SLC6A2) c2(NP_001165973) c3(sodium-dependent noradrenaline transporter isoform 3) c4(68064) c5(68065, 68066, 68067, 68068, 68069, 68070) c6( ) c7(azs, T, bO, bB, ci, lF, aE, iX, pX, K, eL, Jl, aW, g, cm, aiO, aqr, cbM, mx, QF, uJ, wJ, To, ww, ct, aBl, cd, ft, amJ, awb, dL, he, Jj, aq, Jh, hq, ok, cA, dB, uz, jd, B, lI, oe, DV, aA, cz, v, j, q, aT, bK, jj, ae, bkl, ak, avV, blD, s, yL, r, en, ik, aFA, bdn, cT, AH, yH, Fl, awa, VB, blu, gx, pY, iW, OR, bS, ad, av, w, afm, Od, qE, tb, vj, ff, aF, aTR, Z, bz, apM, Ew, bE, eB, ra, hv); #c1(SLC6A2) c2(XP_006721326) c3(sodium-dependent noradrenaline transporter isoform X1) c4(293941) c5(293942, 293943, 293944, 293945, 293946, 293947) c6( ) c7(azs, T, bO, bB, ci, lF, aE, iX, pX, K, eL, Jl, aW, g, cm, aiO, aqr, cbM, mx, QF, uJ, wJ, To, ww, ct, aBl, cd, ft, amJ, awb, dL, he, Jj, aq, Jh, hq, ok, cA, dB, uz, jd, B, lI, oe, DV, aA, cz, v, j, q, aT, bK, jj, ae, bkl, ak, avV, blD, s, yL, r, en, iK, aFA, bdn, cT, AH, yH, Fl, awa, VB, blu, gx, pY, iW, oR, bS, ad, av, w, afm, Od, qE, tb, vj, ff, aF, aTR, Z, bz, apM, Ew, bE, eB, ra, hv); #c1(SLCGA2) c2(XP_011521599) c3(sodium-dependent noradrenaline transporter isoform X3) c4(316405) c5(316406, 316407, 316408, 316409, 316410, 316411) c6( ) c7(azs, T, bO, bB, ci, lF, aE, iX, pX, K, eL, Jl, aW, g, cm, aiO, aqr, cbM, mx, QF, uJ, wJ, To, ww, ct, aBl, cd, ft, amJ, awb, dL, he, Jj, aq, Jh, hq, ok, cA, dB, uz, jd, B, lI, oe, DV, aA, cz, v, j, q, aT, bK, jj, ae, bkl, ak, avV, blD, s, yL, r, en, iK, aFA, bdn, cT, AH, yH, Fl, awa, VB, blu, gx, pY, iW, oR, bS, ad, av, w, afm, Od, qE, tb, vj, ff, aF, aTR, Z, bz, apM, Ew, bE, eB, ra, hv); #c1(SLC6A20) c2(NP_064593) c3(sodium- and chloride-dependent transporter XTRP3 isoform 1) c4(203155) c5(203156, 203157, 203158, 203159, 203160, 203161) c6( ) c7(ey, cFK, cFL, C, ib); #c1(SLC6A20) c2(NP_071800) c3(sodium- and chloride-dependent transporter XTRP3 isoform 2) c4(212042) c5(212043, 212044, 212045, 212046, 212047, 212048) c6( ) c7(ey, cFK, cFL, C, ib); #c1(SLC6A20) c2(XP_005265293) c3(sodium- and chloride-dependent transporter XTRP3 isoform X1) c4(279455) c5(279456, 279457, 279458, 279459, 279460, 279461) c6( ) c7(ey, cFK, cFL, C, ib); #c1(SLC6A4) c2(NP_001036) c3(sodium-dependent serotonin transporter) c4(34115, 34116) c5( ) c6( ) c7(lD, tJ, T, uk, aUk, fR, ahc, cbF, e, al, bjf, bO, gf, ur, Gp, Jt, W, Vp, wM, bhN, cGy, bWj, Fo, iX, Gb, ta, abj, cbf, K, oi, cGF, xa, hm, ly, cGw, zQ, up, amy, aW, hl, Fa, da, bMC, Eg, lm, Jl, eE, mZ, aiO, aaS, bdc, aqr, abS, Kr, cGx, U, ZT, uo, Gs, sv, aTV, abl, cGC, uJ, xh, FW, dD, boK, sQ, aw, n, To, aj, ww, ct, nA, abQ, aPb, cGB, cd, bhM, FY, ke, Zs, h, eV, gl, bWv, mV, mY, Cr, he, aze, wC, mU, abf, PT, Jj, aml, fe, aFx, vo, cM, Gi, lx, mX, Jh, hq, amt, aap, ok, aqp, abV, cA, aUn, uw, yP, Ei, bhU, vL, at, agZ, ut, aqm, uz, bD, lB, jd, mT, qc, bL, bcq, jv, aCw, dA, bMD, iH, He, jx, eX, Nt, cz, mS, ti, cGz, j, v, aql, il, cGE, wy, q, J, aqN, AK, Si, aPW, ga, PW, gM, aqt, HJ, aTZ, hb, aVM, sl, QU, aql, aT, wP, bK, ahg, an, rE, rz, af, aFs, aGP, yL, zS, r, iy, cu, HL, wA, f, PF, cGD, pb, FT, tl, bN, Ji, pH, rR, ac, mG, cbE, aTU, amu, lw, Fl, aen, gO, yv, qX, nl, ra, aFe, akd, aat, acl, ha, uG, Gd, bS, uu, eq, bxb, xt, kL, BY, dP, HF, aqq, ot, sN, mW, oz, aqk, Sb, vc, lQ, bME, na, Jc, buh, tb, ue, Gv, gC, cr, eZ, kD, gl, hp, aTR, EZ, iV, Z, FX, jb, cuy, D, gk, cGA, afN, bMA, lI, bMt, gZ, Rm, clW, aS, ei, cGv, pk, lC, aqw, aqD, gq, rk); #c1(SLC6A5) c2(XP_011518775) c3(sodium- and chloride-dependent glycine transporter 2 isoform X1) c4(313174) c5(313175, 313176, 313177, 313178, 313179, 313180) c6( ) c7(ll, lF, T, aWN, aW, dB, Jl, AH, vj, awa, aT, aA, awb, aF, K, ci, g, cGG, B); #c1(SLC6A6) c2(NP_001127839) c3(sodium- and chloride-dependent taurine transporter isoform c) c4(49333, 49334, 49335, 49336) c5(49337, 49338, 49339, 49340, 49341, 49342, 49343, 49344, 49345, 49346, 49347, 49348, 49349) c6( ) c7(iO, T, jv, lR, vj, r, j, nC, q); #c1(SLC6A6) c2(NP_001127840) c3(sodium- and chloride-dependent taurine transporter isoform b) c4( ) c5(49350, 49351, 49352, 49353, 49354, 49355, 49356, 49357, 49358, 49359) c6( ) c7(iO, T, jv, lR, vj, r, j, nC, q); #c1(SLC6A6) c2(NP_003034) c3(sodium- and chloride-dependent taurine transporter isoform a) c4( ) c5(134601, 134602, 134603, 134604, 134605, 134606, 134607, 134608) c6( ) c7(iO, T, jv, lR, vj, r, j, nC, q); #c1(SLC6A6) c2(XP_006713370) c3(sodium- and chloride-dependent taurine transporter isoform X2) c4( ) c5(287163) c6( ) c7(iO, T, jv, lR, vj, r, j, nC, q); #c1(SLCGA7) c2(NP_055043) c3(sodium-dependent proline transporter) c4(178514) c5(178515, 178516, 178517, 178518, 178519, 178520) c6( ) c7(bM, vj, Xg); #c1(SLC6A8) c2(NP_001136277) c3(sodium- and chloride-dependent creatine transporter 1 isoform 2) c4(54764) c5(54765, 54766, 54767, 54768, 54769, 54770) c6( ) c7(if, ha, NT, aA, bB, n, nA, qN, cd, ad, eW, nc, cjw, Fo, aT, aU, h, K, ca, ake, ae, UF, cH, aW, nf, aF, bzp, Ku, as, bp, cm, dr, bN, mG, Kr, awl, bzW, Uw); #c1(SLC6A8) c2(NP_001136278) c3(sodium- and chloride-dependent creatine transporter 1 isoform 3) c4(54771, 54772) c5(54773, 54774, 54775, 54776, 54777, 54778, 54779, 54780, 54781, 54782, 54783, 54784, 54785, 54786, 54787, 54788, 54789, 54790, 54791, 54792, 54793, 54794, 54795, 54796, 54797) c6( ) c7(if, ha, NT, aA, bB, n, nA, qN, cd, ad, eW, nc, cjw, Fo, aT, aU, h, K, ca, ake, ae, UF, cH, aW, nf, aF, bzp, Ku, as, bp, cm, dr, bN, mG, Kr, awl, bzW, Uw); #c1(SLCGA8) c2(NP_005620) c3(sodium- and chloride-dependent creatine transporter 1 isoform 1) c4(157998, 157999, 158000, 158001) c5(158002, 158003, 158004, 158005, 158006, 158007, 158008, 158009, 158010, 158011, 158012, 158013, 158014) c6( ) c7(if, ha, NT, aA, bB, n, nA, qN, cd, ad, eW, nc, cjw, Fo, aT, aU, h, K, ca, ake, ae, UF, cH, aW, nf, aF, bzp, Ku, as, bp, cm, dr, bN, mG, Kr, awl, bzW, Uw); #c1(SLC6A9) c2(XP_011540319) c3(sodium- and chloride-dependent glycine transporter 1 isoform X1) c4( ) c5(338730, 338731, 338732, 338733, 338734, 338735, 338736, 338737, 338738, 338739, 338740, 338741) c6( ) c7(ke, PT, UZ, cz, gl, aqm, Eg, xa); #c1(SLC7A1) c2(XP_005266564) c3(high affinity cationic amino acid transporter 1 isoform X1) c4(280232, 280233) c5(280234, 280235, 280236, 280237, 280238, 280239, 280240, 280241, 280242, 280243, 280244, 280245) c6( ) c7(qa, T, aaU, ig, bE, aW, if, aT, cR, r, cd, cz, bB, m, bl, an, ae, q, ad, bA); #c1(SLC7A10) c2(NP_062823) c3(asc-type amino acid transporter 1) c4(202153) c5(202154, 202155, 202156, 202157, 202158, 202159) c6( ) c7(beo, aW, aT, bns, fe, Th); #c1(SLC7A11) c2(NP_055146) c3(cystine/glutamate transporter) c4(179435) c5(179436, 179437, 179438, 179439, 179440, 179441) c6( ) c7(T, aC, lE, aW, aT, j, K, eA, cGH); #c1(SLC7A13) c2(NP_620172) c3(solute carrier family 7 member 13) c4(237464) c5(237465, 237466, 237467, 237468, 237469, 237470) c6( ) c7(cQ, j); #c1(SLC7A13) c2(XP_011515169) c3(solute carrier family 7 member 13 isoform X1) c4(308002) c5(308003, 308004, 308005, 308006, 308007, 308008) c6( ) c7(cQ, j); #c1(SLC7A14) c2(NP_066000) c3(probable cationic amino acid transporter) c4(206708) c5(206709, 206710, 206711, 206712, 206713, 206714) c6( ) c7(cGl, nw, nk); #c1(SLC7A2) c2(NP_001158243) c3(cationic amino acid transporter 2 isoform 3) c4(63497) c5(63498, 63499, 63500, 63501, 63502, 63503, 63504, 63505, 63506, 63507) c6( ) c7(aaU, ig, vj, aoo, aj, aXd, C, fq, B, J); #c1(SLC7A2) c2(NP_003037) c3(cationic amino acid transporter 2 isoform 1) c4(134609) c5(134610, 134611, 134612, 134613, 134614, 134615, 134616, 134617, 134618, 134619) c6( ) c7(aaU, ig, vj, aoo, aj, aXd, C, fq, B, J); #c1(SLC7A2) c2(XP_005273668) c3(cationic amino acid transporter 2 isoform X1) c4(284765) c5(284766, 284767, 284768, 284769, 284770, 284771, 284772, 284773) c6( ) c7(aaU, ig, vj, aoo, aj, aXd, C, fq, B, J); #c1(SLC7A3) c2(NP_001041629) c3(cationic amino acid transporter 3) c4(34225) c5(34226, 34227, 34228, 34229, 34230, 34231, 34232, 34233, 34234, 34235) c6( ) c7(bVz); #c1(SLC7A4) c2(NP_004164) c3(cationic amino acid transporter 4) c4(143975, 143976) c5(143977, 143978, 143979, 143980, 143981, 143982, 143983, 143984, 143985, 143986, 143987, 143988, 143989, 143990, 143991, 143992) c6( ) c7(bM, T, fM, iW, RX, jx, jh, asa, fd, n, bh, aHj, mC, zn, NF, x, agd, cGK, pe, h, DY, pk, yF, wr, mR, Of, eZ, Og, S, eE, ho, Wt, asE, Cv, ck, iQ, cj, aS, k, fL, bC, y, cGJ); #c1(SLC7A5) c2(NP_003477) c3(large neutral amino acids transporter small subunit 1) c4(138670) c5(138671, 138672, 138673, 138674, 138675, 138676) c6( ) c7(Mb, T, id, yG, fd, acU, fr, eA, ad, eW, av, iu, jz, py, pX, aT, qU, aXd, ey, ji, ae, beo, d, iE, oV, Y, fJ, ff, en, as, iK, dr, eM, go, aGi, B); #c1(SLC7A6) c2(NP_003974) c3(Y+L amino acid transporter 2) c4(142563, 142564) c5(142565, 142566, 142567, 142568, 142569, 142570, 142571, 142572, 142573, 142574, 142575, 142576, 142577, 142578, 142579) c6( ) c7(T, py, bE, jh, aeu, dT, aXd, ae, ad); #c1(SLC7A6) c2(XP_011521741) c3(Y+L amino acid transporter 2 isoform X2) c4( ) c5(316450) c6( ) c7(T, py, bE, jh, aeu, dT, aXd, ae, ad); #c1(SLC7A7) c2(XP_011535601) c3(Y+L amino acid transporter 1 isoform X1) c4(332764, 332765, 332766, 332767, 332768, 332769, 332770, 332771, 332772, 332773, 332774) c5(332775, 332776, 332777, 332778, 332779, 332780, 332781, 332782, 332783, 332784, 332785, 332786, 332787, 332788, 332789, 332790, 332791, 332792, 332793, 332794, 332795, 332796, 332797, 332798, 332799, 332800, 332801, 332802, 332803, 332804, 332805, 332806, 332807, 332808, 332809, 332810, 332811, 332812, 332813, 332814, 332815, 332816, 332817, 332818, 332819, 332820, 332821, 332822, 332823, 332824, 332825, 332826, 332827, 332828, 332829, 332830, 332831, 332832, 332833, 332834, 332835, 332836, 332837, 332838, 332839, 332840, 332841, 332842, 332843, 332844, 332845, 332846, 332847, 332848, 332849, 332850, 332851, 332852, 332853, 332854, 332855, 332856) c6( ) c7(bL, cGL, beo, py, jh, aeu, dT, v, aXd, ae, rL, ad); #c1(SLC7A8) c2(NP_001253965) c3(large neutral amino acids transporter small subunit 2 isoform c) c4(94251, 94252, 94253) c5(94254, 94255, 94256, 94257, 94258, 94259, 94260, 94261, 94262, 94263, 94264, 94265) c6( ) c7(dP, T, py, ac, iT, vA, aXd, bt, B, op); #c1(SLC7A8) c2(NP_001253966) c3(large neutral amino acids transporter small subunit 2 isoform d) c4(94266) c5(94267, 94268, 94269, 94270, 94271, 94272, 94273) c6( ) c7(dP, T, py, ac, iT, vA, aXd, bt, B, op); #c1(SLC7A8) c2(NP_036376) c3(large neutral amino acids transporter small subunit 2 isoform a) c4(173346, 173347, 173348, 173349, 173350) c5(173351, 173352, 173353, 173354, 173355, 173356, 173357, 173358, 173359, 173360, 173361, 173362, 173363, 173364, 173365, 173366, 173367, 173368, 173369, 173370) c6( ) c7(dP, T, py, ac, iT, vA, aXd, bt, B, op); #c1(SLC7A8) c2(NP_877392) c3(large neutral amino acids transporter small subunit 2 isoform b) c4( ) c5(256725, 256726, 256727, 256728, 256729, 256730, 256731, 256732, 256733, 256734, 256735, 256736, 256737, 256738, 256739) c6( ) c7(dP, T, py, ac, iT, vA, aXd, bt, B, op); #c1(SLC7A9) c2(XP_011524704) c3(B(O,+)-type amino acid transporter 1 isoform X1) c4(319193) c5(319194, 319195, 319196, 319197, 319198, 319199, 319200, 319201, 319202, 319203, 319204, 319205) c6( ) c7(fU, AW, beo, A, eR, af, vj, aw, uz, gA, aj, dy, aXd, cGM, y, wS, J); #c1(SLC8A1) c2(NP_001106271) c3(sodium/calcium exchanger 1 isoform B precursor) c4(42412) c5(42413, 42414, 42415, 42416, 42417, 42418, 42419, 42420, 42421, 42422) c6( ) c7(pY, e, aA, cz, aj, j, cd, J, aJf, Bc, lG, cy, K, bq, eu, vj, aq, uU, aF, qa, ek, ac, hR, HB, cB, B); #c1(SLC8A) c2(NP_001106272) c3(sodium/calcium exchanger 1 isoform C precursor) c4(42423) c5(42424, 42425, 42426, 42427, 42428, 42429) c6( ) c7(pY, e, aA, cz, aj, j, cd, J, aJf, Bc, lG, cy, K, bq, eu, vj, aq, uU, aF, qa, ek, ac, hR, HB, cB, B); #c1(SLC8A1) c2(NP_001239553) c3(sodium/calcium exchanger 1 isoform E precursor) c4(88520) c5(88521, 88522, 88523, 88524, 88525, 88526) c6( ) c7(pY, e, aA, cz, aj, j, cd, J, aJf, Bc, lG, cy, K, bq, eu, vj, aq, uU, aF, qa, ek, ac, hR, HB, cB, B); #c1(SLC8A1) c2(XP_005264571) c3(sodium/calcium exchanger 1 isoform X2) c4(279061, 279062) c5(279063, 279064, 279065, 279066, 279067, 279068, 279069, 279070, 279071, 279072, 279073, 279074, 279075, 279076, 279077, 279078, 279079, 279080, 279081, 279082, 279083, 279084, 279085, 279086) c6( ) c7(pY, e, aA, cz, aj, j, cd, J, aJf, Bc, lG, cy, K, bq, eu, vj, aq, uU, aF, qa, ek, ac, hR, HB, cB, B); #c1(SLC8A1) c2(XP_006712149) c3(sodium/calcium exchanger 1 isoform X11) c4( ) c5(286426, 286427, 286428, 286429, 286430, 286431, 286432, 286433, 286434, 286435, 286436, 286437, 286438, 286439, 286440, 286441, 286442, 286443, 286444, 286445, 286446) c6( ) c7(pY, e, aA, cz, aj, j, cd, J, aJf, Bc, lG, cy, K, bq, eu, vj, aq, uU, aF, qa, ek, ac, hR, HB, cB, B); #c1(SLC8A1) c2(XP_011531358) c3(sodium/calcium exchanger 1 isoform X1) c4(327341) c5(327342, 327343, 327344, 327345, 327346, 327347, 327348, 327349, 327350, 327351, 327352, 327353, 327354, 327355, 327356) c6( ) c7(pY, e, aA, cz, aj, j, cd, J, aJf, Bc, lG, cy, K, bq, eu, vj, aq, uU, aF, qa, ek, ac, hR, HB, cB, B); #c1(SLC8A1) c2(XP_011531360) c3(sodium/calcium exchanger 1 isoform X10) c4(327357, 327358, 327359, 327360) c5(327361, 327362, 327363, 327364, 327365, 327366) c6( ) c7(pY, e, aA, cz, aj, j, cd, J, aJf, Bc, lG, cy, K, bq, eu, vj, aq, uU, aF, qa, ek, ac, hR, HB, cB, B); #c1(SLC8A2) c2(NP_055878) c3(sodium/calcium exchanger 2 precursor) c4(184121, 184122, 184123, 184124) c5(184125, 184126, 184127, 184128, 184129, 184130, 184131, 184132, 184133, 184134, 184135, 184136, 184137, 184138, 184139, 184140, 184141, 184142, 184143, 184144, 184145, 184146, 184147, 184148) c6( ) c7(lG, cB, eA, eu); #c1(SLC8A3) c2(NP_001123889) c3(sodium/calcium exchanger 3 isoform G) c4( ) c5(47976, 47977, 47978, 47979, 47980, 47981) c6( ) c7(lG, eu, cB, ao); #c1(SLC8A3) c2(NP_150287) c3(sodium/calcium exchanger 3 isoform A precursor) c4(228059) c5(228060, 228061, 228062, 228063, 228064, 228065, 228066, 228067, 228068, 228069) c6( ) c7(lG, eu, cB, ao); #c1(SLC8A3) c2(NP_489479) c3(sodium/calcium exchanger 3 isoform B precursor) c4(231012) c5(231013, 231014, 231015, 231016, 231017, 231018) c6( ) c7(lG, eu, cB, ao); #c1(SLC8A3) c2(NP_891977) c3(sodium/calcium exchanger 3 isoform D precursor) c4(257336, 257337, 257338) c5(257339, 257340, 257341, 257342, 257343, 257344, 257345) c6( ) c7(lG, eu, cB, ao); #c1(SLC8A3) c2(NP_891981) c3(sodium/calcium exchanger 3 isoform F) c4( ) c5(257392, 257393, 257394, 257395, 257396) c6( ) c7(lG, eu, cB, ao); #c1(SLC8A3) c2(XP_011535403) c3(sodium/calcium exchanger 3 isoform X2) c4(332422) c5( ) c6( ) c7(lG, eu, cB, ao); #c1(SLC8A3) c2(XP_011535404) c3(sodium/calcium exchanger 3 isoform X1) c4(332423) c5(332424, 332425, 332426, 332427, 332428, 332429, 332430, 332431, 332432, 332433, 332434, 332435) c6( ) c7(lG, eu, cB, ao); #c1(SLC9A1) c2(XP_011540323) c3(sodium/hydrogen exchanger 1 isoform X1) c4(338749, 338750, 338751) c5(338752, 338753, 338754, 338755, 338756, 338757, 338758, 338759, 338760, 338761, 338762, 338763) c6( ) c7(bL, T, dD, eR, ea, if, cz, ajp, dG, j, ci, eq, qN, aY, Fc, cd, eA, Bc, lC, aT, lG, cy, vc, ji, bq, A, d, iU, aW, Y, LZ, blU, fe, iV, Hv, qa, ck, U, hr, gT, bt, ra, B, yv); #c1(SLC9A2) c2(NP_003039) c3(sodium/hydrogen exchanger 2 precursor) c4(134620) c5(134621, 134622, 134623, 134624, 134625, 134626) c6( ) c7(blU, qS, s, cz); #c1(SLC9A3) c2(NP_004165) c3(sodium/hydrogen exchanger 3 isoform 1) c4(143993) c5(143994, 143995, 143996, 143997, 143998, 143999) c6( ) c7(iV, eR, byo, s, vj, qS, aer, jN, aZs, cz, v, cFn, ble, EZ, bq, aY, uC, cFl, ao, aa); #c1(SLC9A3RI) c2(NP_004243) c3(Na(+)/H(+) exchange regulatory cofactor NHE-RF1) c4(144560, 144561) c5(144562, 144563, 144564, 144565, 144566, 144567, 144568, 144569, 144570, 144571, 144572, 144573) c6( ) c7(T, cGN, ci, q, eA, Ot, aT, h, ji, K, A, d, aW, hl, cr, r, nY, aq, fe, cGO, iR, eE, dh, ra, B, ao); #c1(SLC9A3R2) c2(NP_001123484) c3(Na(+)/H(+) exchange regulatory cofactor NHE-RF2 isoform a) c4(47153) c5(47154, 47155, 47156, 47157, 47158, 47159) c6( ) c7(T, aAf, Ol, aA, cz, bB, fr, ci, eA, aE, Tf, aT, kf, h, ahE, B, s, aW, ff, aq, aF, ahL, en, g, iK, eM, bE, m, X, hS); #c1(SLC9A3R2) c2(NP_004776) c3(Na(+)/H(+) exchange regulatory cofactor NHE-RF2 isoform b) c4(149526) c5(149527, 149528, 149529, 149530, 149531, 149532) c6( ) c7(T, aAf, Dl, aA, cz, bB, fr, ci, eA, aE, Tf, aT, kf, h, ahE, B, s, aW, ff, aq, aF, ahL, en, g, iK, eM, bE, m, X, hS); #c1(SLC9A3R2) c2(XP_005255759) c3(Na(+)/H(+) exchange regulatory cofactor NHE-RF2 isoform X1) c4( ) c5(273849) c6( ) c7(T, aAf, Ol, aA, cz, bB, fr, ci, eA, aE, Tf, aT, kf, h, ahE, B, s, aW, ff, aq, aF, ahL, en, g, iK, eM, bE, m, X, hS); #c1(SLC9A4) c2(NP_001011552) c3(sodium/hydrogen exchanger 4 precursor) c4(24611) c5(24612, 24613, 24614, 24615, 24616, 24617) c6( ) c7(hp); #c1(SLC9A5) c2(NP_004585) c3(sodium/hydrogen exchanger 5 precursor) c4(147904, 147905) c5(147906, 147907, 147908, 147909, 147910, 147911, 147912, 147913) c6( ) c7(eR, cuY, U, blU, cGP, aY); #c1(SLC9A5) c2(XP_011521606) c3(sodium/hydrogen exchanger 5 isoform X4) c4( ) c5(316412, 316413, 316414, 316415, 316416) c6( ) c7(eR, cuY, U, blU, cGP, aY); #c1(SLC9A6) c2(NP_001036002) c3(sodium/hydrogen exchanger 6 isoform a precursor) c4(33656) c5(33657, 33658, 33659, 33660, 33661, 33662, 33663) c6( ) c7(eR, tp, LX, n, v, fu, nA, cGV, C, pU, bjQ, auu, cxz, aR, cd, cGT, afm, blb, cGU, nm, cGQ, ey, cGS, ao, Cw, Ue, cjk, nf, cGX, cGY, anW, QK, Dm, bN, ek, cGW, cGR, agl, Zn, fD, Wl, aFf, LT); #c1(SLC9A6) c2(NP_006350) c3(sodium/hydrogen exchanger 6 isoform b precursor) c4(164256) c5(164257, 164258, 164259, 164260, 164261, 164262) c6( ) c7(eR, tp, LX, n, v, fu, nA, cGV, C, pU, bjQ, auu, cxz, aR, cd, cGT, afm, blb, cGU, nm, cGQ, ey, cGS, ao, Cw, Ue, cjk, nf, cGX, cGY, anW, QK, Dm, bN, ek, cGW, cGR, agl, Zn, fD, Wl, aFf, LT); #c1(SLC9A6) c2(XP_006724789) c3(sodium/hydrogen exchanger 6 isoform X1) c4(296336) c5(296337, 296338, 296339, 296340, 296341) c6( ) c7(eR, tp, LX, n, v, fu, nA, cGV, C, pU, bjQ, auu, cxz, aR, cd, cGT, afm, blb, cGU, nm, cGQ, ey, cGS, ao, Cw, Ue, cjk, nf, cGX, cGY, anW, QK, Dm, bN, ek, cGW, cGR, agl, Zn, fD, Wl, aFf, LT); #c1(SLC9A7) c2(NP_115980) c3(sodium/hydrogen exchanger 7 isoform 2 precursor) c4(225064) c5(225065, 225066, 225067, 225068, 225069, 225070) c6( ) c7(nf, cGR, cGQ, agl); #c1(SLC9A8) c2(NP_001247420) c3(sodium/hydrogen exchanger 8 isoform 1) c4(93518) c5(93519, 93520, 93521, 93522, 93523, 93524, 93525) c6( ) c7(ble); #c1(SLC9A8) c2(NP_056081) c3(sodium/hydrogen exchanger 8 isoform 2) c4(185567, 185568, 185569) c5(185570, 185571, 185572, 185573, 185574, 185575, 185576, 185577, 185578, 185579, 185580) c6( ) c7(ble); #c1(SLC9A8) c2(XP_011527041) c3(sodium/hydrogen exchanger 8 isoform X5) c4( ) c5(321359) c6( ) c7(ble); #c1(SLC9A8) c2(XP_011527046) c3(sodium/hydrogen exchanger 8 isoform X10) c4( ) c5(321360, 321361, 321362, 321363, 321364) c6( ) c7(ble); #c1(SLC9A9) c2(NP_775924) c3(sodium/hydrogen exchanger 9) c4(250711, 250712) c5(250713, 250714, 250715, 250716, 250717, 250718, 250719, 250720, 250721, 250722, 250723, 250724) c6( ) c7(oe, ht, ff, aj, j, en, bN, cGZ); #c1(SLC9B1) c2(NP_001094344) c3(sodium/hydrogen exchanger 981 isoform 2) c4(40680) c5(40681, 40682, 40683, 40684, 40685, 40686) c6( ) c7(Ok, wR, aiV, K, aj); #c1(SLC9B1) c2(XP_011529925) c3(sodium/hydrogen exchanger 9B1 isoform X1) c4(325711, 325712, 325713) c5(325714, 325715, 325716, 325717, 325718, 325719) c6( ) c7(Ok, wR, aiV, K, aj); #c1(SLC9B1) c2(XP_011529926) c3(sodium/hydrogen exchanger 9B1 isoform X2) c4( ) c5(325720, 325721, 325722, 325723, 325724, 325725, 325726, 325727, 325728, 325729, 325730, 325731) c6( ) c7(Ok, wR, aiV, K, aj); #c1(SLC9B1) c2(XP_011529928) c3(sodium/hydrogen exchanger 981 isoform X4) c4(325732) c5( ) c6( ) c7(Ok, wR, aiV, K, aj); #c1(SLC9BI) c2(XP_011529929) c3(sodium/hydrogen exchanger 9B1 isoform X5) c4( ) c5(325733, 325734, 325735, 325736, 325737, 325738) c6( ) c7(Ok, wR, aiV, K, aj); #c1(SLC9B2) c2(NP_001287685) c3(mitochondrial sodium/hydrogen exchanger 982 isoform 3) c4(112537) c5(112538, 112539, 112540, 112541, 112542, 112543) c6( ) c7(qa, cz); #c1(SLC9B2) c2(XP_005262815) c3(mitochondrial sodium/hydrogen exchanger 982 isoform X3) c4( ) c5(278101, 278102, 278103, 278104, 278105, 278106, 278107, 278108, 278109, 278110, 278111, 278112) c6( ) c7(qa, cz); #c1(SLC982) c2(XP_006714148) c3(mitochondrial sodium/hydrogen exchanger 9B2 isoform X1) c4(287580, 287581, 287582) c5(287583, 287584, 287585, 287586, 287587, 287588, 287589, 287590, 287591, 287592) c6( ) c7(qa, cz); #c1(SLC982) c2(XP_006714149) c3(mitochondrial sodium/hydrogen exchanger 982 isoform X2) c4(287593) c5(287594, 287595, 287596, 287597, 287598, 287599) c6( ) c7(qa, cz); #c1(SLC9C1) c2(NP_898884) c3(sodium/hydrogen exchanger 10) c4(257837) c5(257838, 257839, 257840, 257841, 257842, 257843) c6( ) c7(bL, A, lR, eR, cr, cz fe, cy, dy, K, aY, vl, qa); #c1(SLC9C1) c2(XP_011511018) c3(sodium/hydrogen exchanger 10 isoform X5) c4(301460) c5(301461, 301462, 301463, 301464, 301465, 301466) c6( ) c7(bL, A, lR, eR, cr, cz, fe, cy, dy, K, aY, vl, qa); #c1(SLC9C2) c2(NP_848622) c3(sodium/hydrogen exchanger (1) c4(254015) c5(254016, 254017, 254018, 254019, 254020, 254021) c6( ) c7(W, ad, cr); #c1(SLC9C2) c2(XP_011507729) c3(sodium/hydrogen exchanger 11 isoform X4) c4(297809) c5( ) c6( ) c7(W, ad, cr); #c1(SLC9C2) c2(XP_011507732) c3(sodium/hydrogen exchanger 11 isoform X5) c4( ) c5(297810) c6( ) c7(W, ad, cr); #c1(SLC9C2) c2(XP_011507735) c3(sodium/hydrogen exchanger 11 isoform X7) c4( ) c5(297811, 297812, 297813, 297814, 297815) c6( ) c7(W, ad, cr); #c1(SLC01A2) c2(NP_602307) c3(solute carrier organic anion transporter family member 1A2) c4(235692, 235693, 235694, 235695, 235696, 235697, 235698, 235699, 235700) c5(235701, 235702, 235703, 235704, 235705, 235706, 235707, 235708, 235709, 235710, 235711, 235712, 235713, 235714, 235715, 235716, 235717, 235718, 235719, 235720, 235721, 235722, 235723, 235724, 235725, 235726, 235727, 235728, 235729, 235730, 235731, 235732, 235733, 235734, 235735, 235736, 235737, 235738, 235739, 235740, 235741, 235742, 235743, 235744, 235745, 235746, 235747, 235748, 235749, 235750, 235751, 235752, 235753, 235754, 235755, 235756, 235757, 235758, 235759, 235760, 235761, 235762, 235763) c6( ) c7(bL, T, l, A, u, byF, aW, kK, ha, aT, cz, ci, act, iV, iK, ae, ad); #c1(SLC01A2) c2(XP_011519122) c3(solute carrier organic anion transporter family member 1A2 isoform X2) c4(313725) c5(313726, 313727, 313728, 313729, 313730, 313731) c6( ) c7(bL, T, l, A, u, byF, aW, kK, ha, aT, cz, ci, act, iV, ik, ae, ad); #c1(SLC01B1) c2(NP_006437) c3(solute carrier organic anion transporter family member 181) c4(165225) c5(165226, 165227, 165228, 165229, 165230, 165231) c6( ) c7(bL, gV, T, qo, aw, cz, sM, n, aj, v, j, qf, bB, fr, x, cHa, l, cc, u, ec, lv, byF, fS, gd, aT, oj, h, ux, dQ, bc, s, cH, aW, hC, hl, aq, aB, iR, es, eM, bE, wE, k, m, hE, B, y); #c1(SLC01B3) c2(NP_062818) c3(solute carrier organic anion transporter family member 183) c4(202124, 202125) c5(202126, 202127, 202128, 202129, 202130, 202131, 202132, 202133, 202134, 202135, 202136, 202137, 202138, 202139, 202140, 202141, 202142, 202143, 202144, 202145) c6( ) c7(gV, T, ex, bB, C, aY, q, ad, cHa, av, aE, aT, lG, eV, ae, bc, B, iU, hG, aW, Lf, hC, PA, hl, r, aq, g, bu, hJ, aAW, ar, rR, eM, bE, hk, gX, aUS, np, m, adg, X); #c1(SLC01B7) c2(NP_001009562) c3(putative solute carrier organic anion transporter family member 187) c4(23604, 23605) c5(23606, 23607, 23608, 23609, 23610, 23611, 23612, 23613, 23614, 23615, 23616, 23617) c6( ) c7(l, u); #c1(SLC01C1) c2(NP_001139417) c3(solute carrier organic anion transporter family member IC1 isoform 3) c4(59306) c5(59307, 59308, 59309, 59310, 59311, 59312) c6( ) c7(bS, ct, Z, gT, y, bA); #c1(SLC01C1) c2(NP_001139418) c3(solute carrier organic anion transporter family member IC1 isoform 1) c4(59313, 59314) c5(59315, 59316, 59317, 59318, 59319, 59320, 59321, 59322, 59323, 59324, 59325, 59326, 59327, 59328, 59329, 59330) c6( ) c7(bS, ct, Z, gT, y, bA); #c1(SLC01C1) c2(NP_059131) c3(solute carrier organic anion transporter family member IC1 isoform 2) c4(193202) c5(193203, 193204, 193205, 193206, 193207, 193208) c6( ) c7(bS, ct, Z, gT, y, bA); #c1(SLC02A1) c2(NP_005621) c3(solute carrier organic anion transporter family member 2A1) c4(158015, 158016) c5(158017, 158018, 158019, 158020, 158021, 158022, 158023, 158024, 158025, 158026, 158027, 158028) c6( ) c7(s, fV, vj, hC, aBN, r, cHb, pg, ci, avG, iK, q); #c1(SLC02A1) c2(XP_011511392) c3(solute carrier organic anion transporter family member 2A1 isoform X1) c4(301965) c5(301966, 301967, 301968, 301969, 301970, 301971) c6( ) c7(s, fV, vj, hC, aBN, r, cHb, pg, ci, avG, ik, q); #c1(SLC02B1) c2(NP_001138683) c3(solute carrier organic anion transporter family member 281 isoform 2) c4(57405, 57406, 57407, 57408, 57409, 57410) c5(57411, 57412, 57413, 57414, 57415, 57416, 57417, 57418, 57419, 57420, 57421, 57422, 57423, 57424, 57425, 57426, 57427, 57428, 57429, 57430, 57431, 57432, 57433, 57434, 57435, 57436) c6( ) c7(hd, aE, nB, aW, hC, aT, aq, iR, iK, ae, ad); #c1(SLC02B1) c2(NP_001138684) c3(solute carrier organic anion transporter family member 2B1 isoform 3) c4(57437) c5(57438, 57439, 57440, 57441, 57442, 57443, 57444, 57445, 57446, 57447, 57448, 57449, 57450, 57451, 57452, 57453) c6( ) c7(hd, aE, nB, aW, hC, aT, aq, iR, iK, ae, ad); #c1(SLC02B1) c2(NP_009187) c3(solute carrier organic anion transporter family member 2B1 isoform 1) c4(171383, 171384) c5(171385, 171386, 171387, 171388, 171389, 171390, 171391, 171392, 171393, 171394, 171395, 171396, 171397, 171398, 171399, 171400, 171401, 171402, 171403, 171404, 171405, 171406, 171407, 171408) c6( ) c7(hd, aE, nB, aW, hC, aT, aq, iR, ik, ae, ad); #c1(SLC03A1) c2(NP_001138516) c3(solute carrier organic anion transporter family member 3A1 isoform 2) c4(56919) c5(56920, 56921, 56922, 56923, 56924, 56925) c6( ) c7(T, aE, aW, cz, aT, k, bX); #c1(SLC03A1) c2(NP_037404) c3(solute carrier organic anion transporter family member 3A1 isoform 1) c4(175334) c5(175335, 175336, 175337, 175338, 175339, 175340) c6( ) c7(T, aE, aW, cz, aT, k, bX); #c1(SLC04A1) c2(NP_057438) c3(solute carrier organic anion transporter family member 4A1) c4(190801, 190802, 190803, 190804) c5(190805, 190806, 190807, 190808, 190809, 190810, 190811, 190812, 190813, 190814, 190815, 190816, 190817, 190818, 190819, 190820, 190821, 190822, 190823, 190824, 190825, 190826, 190827, 190828, 190829, 190830, 190831, 190832) c6( ) c7(aE, c, m, t, eJ, vk); #c1(SLC04A1) c2(XP_005260260) c3(solute carrier organic anion transporter family member 4A1 isoform X5) c4( ) c5(276501, 276502, 276503, 276504, 276505, 276506) c6( ) c7(aE, c, m, t, eJ, vk); #c1(SLC04A1) c2(XP_011527095) c3(solute carrier organic anion transporter family member 4A1 isoform X3) c4(321568) c5( ) c6( ) c7(aE, c, m, t, eJ, vk); #c1(SLC04C1) c2(NP_851322) c3(solute carrier organic anion transporter family member 4C1) c4(254562) c5(254563, 254564, 254565, 254566, 254567, 254568) c6( ) c7(cz); #c1(SLC05A1) c2(NP_001139480) c3(solute carrier organic anion transporter family member 5A1 isoform 2) c4(59413) c5(59414, 59415, 59416, 59417, 59418, 59419) c6( ) c7(iR, aW, aw, aq, aT); #c1(SLC05A1) c2(NP_001139481) c3(solute carrier organic anion transporter family member 5A1 isoform 3) c4(59420) c5(59421, 59422, 59423, 59424, 59425, 59426) c6( ) c7(iR, aW, aw, aq, aT); #c1(SLC05A1) c2(NP_112220) c3(solute carrier organic anion transporter family member 5A1 isoform 1) c4(220890) c5(220891, 220892, 220893, 220894, 220895, 220896) c6( ) c7(iR, aW, aw, aq, aT); #c1(SLC06A1) c2(NP_001275931) c3(solute carrier organic anion transporter family member 6A1 isoform 1) c4(107422, 107423) c5(107424, 107425, 107426, 107427, 107428, 107429, 107430, 107431, 107432, 107433, 107434, 107435, 107436, 107437, 107438, 107439, 107440) c6( ) c7(bM, T, qo, pf, ea, if, e, al, fd, bB, qj, ci, VV, x, aE, u, dV, K, bU, hu, hH, dF, eL, azh, eG, dH, Ow, aW, Jl, iE, g, as, eE, eF, cm, bw, yd, xK, eM, nB, cj, dW, aZo, k, m, cE, dD, gU, aWG, ht, aw, n, aj, fr, qN, pM, ai, br, b, ft, aMV, h, eV, hn, dL, vd, hB, he, dx, A, asp, sp, CM, AQ, bcd, ge, fe, aq, bFN, pm, dw, kk, Ld, iO, W, bP, asl, dB, uw, at, bFO, it, B, ayV, bL, eR, aZy, Dh, yk, jx, eX, aA, cz, fF, v, j, do, q, J, eA, hb, aT, ae, af, bA, qw, avV, s, iU, Y, Lf, afS, r, lo, en, bu, t, iK, pb, zJ, ck, sS, nM, ac, c, ig, Nk, gX, wR, bt, rA, ao, hS, gx, id, akd, HM, jh, bs, lD, oZ, oH, Gn, eq, hF, ad, hX, dP, av, l, hd, oW, aNp, ji, d, lU, bFM, ff, eH, aZw, cr, eZ, Ej, aF, xH, S, bFP, iV, bz, Ga, qa, ek, es, bE, gZ, i, hE, iu, y, pC); #c1(SLC06A1) c2(NP_001275933) c3(solute carrier organic anion transporter family member 6A1 isoform 2) c4(107460) c5(107461, 107462, 107463, 107464, 107465, 107466) c6( ) c7(bM, T, qo, pf, ea, if, e, al, fd, bB, qj, ci, W, x, aE, u, dV, K, bU, hu, hH, dF, eL, azh, eG, dH, Ow, aW, Jl, iE, g, as, eE, eF, cm, bw, yd, xK, eM, nB, cj, dW, aZo, k, m, cE, dD, gU, aWG, ht, aw, n, aj, fr, qN, pM, ai, br, b, ft, aMV, h, eV, hn, dL, vd, hB, he, dx, A, asp, sp, CM, AQ, bcd, ge, fe, aq, bFN, pm, dw, kk, Ld, iO, W, bP, asl, dB, uw, at, bFO, it, B, ayV, bL, eR, aZy, Dh, yk, jx, eX, aA, cz, fF, v, j, Qo, q, J, eA, hb, aT, ae, af, bA, qw, avV, s, iU, Y, Lf, afS, r, lo, en, bu, t, ik, pb, zJ, ck, sS, nM, ac, c, ig, Nk, gX, wR, bt, rA, ao, hS, gx, id, akd, HM, jh, bs, lD, oZ, oH, Gn, eq, hF, ad, hX, dP, av, l, hd, oW, aNp, ji, d, lU, bFM, ff, eH, aZw, cr, eZ, Ej, aF, xH, S, bFP, iV, bz, Ga, qa, ek, es, bE, gZ, i, hE, iu, y, pC); #c1(SLC06A1) c2(XP_011541455) c3(solute carrier organic anion transporter family member 6A1 isoform X7) c4( ) c5(340037) c6( ) c7(bM, T, qo, pf, ea, if, e, al, fd, bB, qj, ci, VV, x, aE, u, dV, K, bU, hu, hH, dF, eL, azh, eG, dH, Ow, aW, Jl, iE, g, as, eE, eF, cm, bw, yd, xK, eM, nB, cj, dW, aZo, k, m, cE, dD, gU, aWG, ht, aw, n, aj, fr, qN, pM, ai, br, b, ft, aMV, h, eV, hn, dL, vd, hB, he, dx, A, asp, sp, CM, AQ, bcd, ge, fe, aq, bFN, pm, dw, kk, Ld, iO, W, bP, asl, dB, uw, at, bFO, it, B, ayV, bL, eR, aZy, Dh, yk, jx, eX, aA, cz, fF, v, j, do, q, J, eA, hb, aT, ae, af, bA, qw, avV, s, iU, Y, Lf, afS, r, lo, en, bu, t, iK, pb, zJ, ck, sS, nM, ac, c, ig, Nk, gX, wR, bt, rA, ao, hS, gx, id, akd, HM, jh, bs, lD, oZ, OH, Gn, eq, hF, ad, hX, dP, av, l, hd, oW, aNp, ji, d, lU, bFM, ff, eH, aZw, cr, eZ, Ej, aF, xH, S, bFP, iV, bz, Ga, qa, ek, es, bE, gZ, i, hE, iu, y, pC); #c1(SLFN12) c2(NP_001275938) c3(schlafen family member 12) c4(107467, 107468, 107469, 107470) c5(107471, 107472, 107473, 107474, 107475, 107476, 107477, 107478, 107479, 107480, 107481, 107482, 107483, 107484, 107485, 107486, 107487, 107488, 107489, 107490, 107491, 107492, 107493, 107494, 107495, 107496, 107497, 107498, 107499) c6( ) c7(li, T, eU, bE, baT, bB, Qc); #c1(SLFN12L) c2(XP_011522471) c3(schlafen family member 12-like isoform X1) c4(317098, 317099) c5(317100, 317101, 317102, 317103, 317104, 317105, 317106, 317107, 317108, 317109, 317110, 317111, 317112, 317113) c6( ) c7(h, Y, A); #c1(SLFN14) c2(NP_001123292) c3(schlafen family member (4) c4(46862) c5(46863, 46864, 46865, 46866, 46867, 46868) c6( ) c7(Y); #c1(SLFN5) c2(NP_659412) c3(schlafen family member 5) c4(239778) c5(239779, 239780, 239781, 239782, 239783, 239784) c6( ) c7(Y); #c1(SLFN5) c2(XP_005257991) c3(schlafen family member 5 isoform X1) c4(275442) c5(275443, 275444, 275445, 275446, 275447, 275448) c6( ) c7(Y); #c1(SLIT1) c2(NP_003052) c3(slit homolog 1 protein precursor) c4(134690, 134691, 134692, 134693, 134694, 134695) c5(134696, 134697, 134698, 134699, 134700, 134701, 134702, 134703, 134704, 134705, 134706, 134707, 134708, 134709, 134710, 134711, 134712, 134713, 134714, 134715, 134716, 134717, 134718, 134719, 134720, 134721, 134722, 134723, 134724, 134725, 134726, 134727, 134728, 134729, 134730, 134731, 134732, 134733, 134734, 134735, 134736, 134737, 134738, 134739, 134740, 134741, 134742, 134743, 134744, 134745, 134746, 134747, 134748, 134749, 134750) c6(134751, 134752, 134753, 134754, 134755, 134756, 134757, 134758, 134759, 134760, 134761, 134762, 134763, 134764) c7(dP, T, hd, nB, hb, aW, r, aT, aA, aF, np, y, bz, eA); #c1(SLIT2) c2(NP_001276064) c3(slit homolog 2 protein isoform 2 precursor) c4(107844, 107845) c5(107846, 107847, 107848, 107849, 107850, 107851, 107852, 107853, 107854, 107855, 107856, 107857) c6( ) c7(Mb, T, aC, fG, if, ha, aA, n, fr, ci, qN, il, AO, q, ad, eA, Bl, ft, aE, wG, ja, aT, h, ae, jf, Yj, MD, qt, aW, ff, r, fe, kp, af, en, g, t, cm, bz, ac, dB, hk, np, m, yA, ME, adg, B); #c1(SLIT2) c2(NP_001276065) c3(slit homolog 2 protein isoform 3 precursor) c4(107858) c5(107859, 107860, 107861, 107862, 107863, 107864) c6( ) c7(Mb, T, aC, fG, if, ha, aA, n, fr, ci, qN, il, AD, q, ad, eA, Bl, ft, aE, wG, ja, aT, h, ae, jf, Yj, MD, qt, aW, ff, r, fe, kp, aF, en, g, t, cm, bz, ac, dB, hk, np, m, yA, ME, adg, B); #c1(SLIT2) c2(NP_004778) c3(slit homolog 2 protein isoform 1 precursor) c4(149547) c5(149548, 149549, 149550, 149551, 149552, 149553) c6( ) c7(Mb, T, aC, fG, if, ha, aA, n, fr, ci, qN, il, AD, q, ad, eA, Bl, ft, aE, wG, ja, aT, h, ae, jf, Yj, MD, qt, aW, ff, r, fe, kp, aF, en, g, t, cm, bz, ac, dB, hk, np, m, yA, ME, adg, B); #c1(SLIT3) c2(NP_003053) c3(slit homolog 3 protein isoform 2 precursor) c4(134765, 134766) c5(134767, 134768, 134769, 134770, 134771, 134772, 134773, 134774, 134775, 134776, 134777, 134778) c6( ) c7(Mb, T, aC, if, ha, aA, n, v, fr, qN, il, AO, q, ad, eA, dP, Bl, hd, aE, wG, ja, aT, h, an, ae, bA, jf, Yj, MD, qt, aW, ff, r, fe, kp, af, en, g, t, cm, bz, fq, nB, dB, hk, wk, np, m, yA, adg, y); #c1(SLITRK1) c2(NP_001268432) c3(SLIT and NTRK-like protein 1 precursor) c4(99930) c5(99931, 99932, 99933, 99934, 99935, 99936) c6( ) c7(rE, Wi, yL, he, qc); #c1(SLITRK2) c2(NP_115928) c3(SLIT and NTRK-like protein 2 precursor) c4(224748, 224749) c5(224750, 224751, 224752, 224753, 224754, 224755, 224756, 224757, 224758, 224759, 224760, 224761, 224762, 224763, 224764, 224765, 224766, 224767, 224768, 224769, 224770, 224771, 224772, 224773, 224774, 224775, 224776, 224777, 224778, 224779, 224780, 224781, 224782, 224783, 224784, 224785, 224786) c6( ) c7(bK); #c1(SLITRK3) c2(NP_055741) c3(SLIT and NTRK-like protein 3 precursor) c4(183203, 183204) c5(183205, 183206, 183207, 183208, 183209, 183210, 183211, 183212, 183213, 183214, 183215, 183216, 183217, 183218, 183219, 183220) c6( ) c7(oe, cd); #c1(SLITRK5) c2(NP_056382) c3(SLIT and NTRK-like protein 5 precursor) c4(187356, 187357) c5(187358, 187359, 187360, 187361, 187362, 187363, 187364, 187365, 187366, 187367, 187368, 187369) c6( ) c7(qc, hq); #c1(SLITRK6) c2(NP_115605) c3(SLIT and NTRK-like protein 6 precursor) c4(223361) c5(223362, 223363, 223364, 223365, 223366, 223367) c6( ) c7(cHc, Be); #c1(SLK) c2(NP_001291672) c3(STE20-like serine/threonine-protein kinase isoform 2) c4(116305) c5(116306, 116307, 116308, 116309, 116310, 116311) c6( ) c7(T, aW, ff, aT, lG, zC, en, lB, ae); #c1(SLK) c2(NP_055535) c3(STE20-like serine/threonine-protein kinase isoform 1) c4(181812) c5(181813, 181814, 181815, 181816, 181817, 181818) c6( ) c7(T, aW, ff, aT, lG, zC, en, lB, ae); #c1(SLMAP) c2(NP_001291349) c3(sarcolemmal membrane-associated protein isoform a) c4(115683) c5(115684, 115685, 115686, 115687, 115688, 115689) c6( ) c7(Bm, uY, hW); #c1(SLMAP) c2(NP_001291350) c3(sarcolemmal membrane-associated protein isoform c) c4(115690, 115691) c5(115692, 115693, 115694, 115695, 115696, 115697, 115698, 115699, 115700, 115701, 115702, 115703, 115704) c6( ) c7(Bm, uY, hW); #c1(SLMAP) c2(NP_001291351) c3(sarcolemmal membrane-associated protein isoform d) c4( ) c5(115705, 115706, 115707, 115708, 115709) c6( ) c7(Bm, uY, hW); #c1(SLMAP) c2(NP_001291352) c3(sarcolemmal membrane-associated protein isoform e) c4(115710) c5(115711, 115712, 115713, 115714, 115715) c6( ) c7(Bm, uY, hW); #c1(SLMAP) c2(NP_009090) c3(sarcolemmal membrane-associated protein isoform b) c4(170614) c5(170615, 170616, 170617, 170618, 170619, 170620, 170621, 170622, 170623, 170624) c6( ) c7(Bm, uY, hW); #c1(SLMAP) c2(XP_005265515) c3(sarcolemmal membrane-associated protein isoform X7) c4(279569) c5( ) c6( ) c7(Bm, uY, HW); #c1(SLMAP) c2(XP_005265516) c3(sarcolemmal membrane-associated protein isoform X8) c4(279570) c5( ) c6( ) c7(Bm, uY, hW); #c1(SLMAP) c2(XP_005265521) c3(sarcolemmal membrane-associated protein isoform X1l) c4(279571, 279572) c5(279573, 279574, 279575, 279576, 279577, 279578, 279579, 279580, 279581, 279582, 279583, 279584, 279585) c6( ) c7(Bm, uY, hW); #c1(SLMAP) c2(XP_005265529) c3(sarcolemmal membrane-associated protein isoform X19) c4(279586) c5( ) c6( ) c7(Bm, uY, hW); #c1(SLMAP) c2(XP_006713389) c3(sarcolemmal membrane-associated protein isoform X18) c4( ) c5(287165) c6( ) c7(Bm, uY, hW); #c1(SLMAP) c2(XP_011532407) c3(sarcolemmal membrane-associated protein isoform X20) c4( ) c5(328595, 328596, 328597, 328598, 328599, 328600, 328601) c6( ) c7(Bm, uY, hW); #c1(SLM02) c2(NP_057129) c3(protein slowmo homolog 2 isoform 1) c4(189039) c5(189040, 189041, 189042, 189043, 189044, 189045) c6( ) c7(aC); #c1(SLN) c2(NP_003054) c3(sarcolipin) c4(134779) c5(134780, 134781, 134782, 134783, 134784, 134785, 134786, 134787, 134788, 134789, 134790, 134791, 134792, 134793) c6( ) c7(T, mr, acl, if, akz, bB, n, akA, v, j, qN, cd, aT, aua, Y, aW, EH, Dy, bE, bwE, uS, bD); #c1(SLP1) c2(NP_003055) c3(antileukoproteinase precursor) c4(134794, 134795) c5(134796, 134797, 134798, 134799, 134800, 134801, 134802, 134803) c6(134804, 134805) c7(lD, T, aC, iV, fR, aD, jh, aA, bB, cHd, afK, g, fr, fd, q, eW, BY, dP, av, cfF, cfE, Tl, Cj, aE, WY, pX, ld, kl, aT, ab, h, ji, MO, nJ, bc, aa, ly, Ck, xR, vT, HY, aW, TH, ff, eH, hl, Ce, aq, aaE, kp, aF, en, Ca, anW, t, cl, bz, Ci, pb, DF, lx, eF, ik, D, y, iQ, ac, eM, aBs, TJ, bE, TG, au, X, hR, at, rx, CZ, aS, m, lC, rA, Vj, B, ao); #c1(SLTM) c2(NP_001013865) c3(SAFB-like transcription modulator isoform b) c4(25796) c5( ) c6( ) c7(T, aC, aCh, pf, bll, bO, fd, bB, ci, aY, bv, aE, Xc, pX, Qk, K, KC, eG, iE, aW, g, as, eF, ir, bw, QA, Qt, iP, eM, iA, aep, m, Xu, ik, n, fr, rP, ft, gd, kH, dL, kj, aq, kp, Qy, SE, dB, ayS, hL, auh, it, B, dA, eR, jx, aA, aNG, lu, q, eA, aT, ahg, rz, ae, jf, s, nO, oV, Y, r, en, aDY, cl, t, Vi, iK, bN, Qi, nM, iM, np, bt, ao, VB, hS, id, LX, bXc, jh, KO, Sv, Fc, aue, ad, dP, av, hd, ib, py, Db, hP, d, bug, je, PE, ff, eH, gl, aF, adj, jb, bz, bF, es, RV, bE, lf, iu); #c1(SLTM) c2(NP_079031) c3(SAFB-like transcription modulator isoform a) c4(216666) c5(216667, 216668, 216669, 216670, 216671) c6( ) c7(T, aC, aCh, pf, bll, bO, fd, bB, ci, aY, bv, aE, Xc, pX, Qk, K, KC, eG, iE, aW, g, as, eF, ir, bw, QA, Qt, iP, eM, iA, aep, m, Xu, ik, n, fr, rP, ft, gd, kH, dL, kj, aq, kp, Dy, SE, dB, ayS, hL, auh, it, B, dA, eR, jx, aA, aNG, lu, q, eA, aT, ahg, rz, ae, jf, s, nO, oV, Y, r, en, aDY, cl, t, Vi, iK, bN, Qi, nM, iM, np, bt, ao, VB, hS, id, LX, bXc, jh, KO, Sv, Fc, aue, ad, dP, av, hd, ib, py, Db, hP, d, bug, je, PE, ff, eH, gl, aF, adj, jb, bz, bF, es, RV, bE, lf, iu); #c1(SLTM) c2(XP_006720753) c3(SAFB-like transcription modulator isoform X12) c4(293663) c5(293664, 293665, 293666, 293667, 293668, 293669, 293670, 293671, 293672, 293673, 293674, 293675) c6( ) c7(T, aC, aCh, pf, bll, bO, fd, bB, ci, aY, bv, aE, Xc, pX, Qk, K, KC, eG, iE, aW, g, as, eF, ir, bw, QA, Qt, iP, eM, iA, aep, m, Xu, ik, n, fr, rP, ft, gd, kH, dL, kj, aq, kp, Qy, SE, dB, ayS, hL, auh, it, B, dA, eR, jx, aA, aNG, lu, q, eA, aT, ahg, rz, ae, jf, s, nO, oV, Y, r, en, aDY, cl, t, Vi, iK, bN, Qi, nM, iM, np, bt, ao, VB, hS, id, LX, bXc, jh, KO, Sv, Fc, aue, ad, dP, av, hd, ib, py, Db, hP, d, bug, je, PE, ff, eH, gl, af, adj, jb, bz, bF, es, RV, bE, lf, iu); #c1(SLTM) c2(XP_011520332) c3(SAFB-like transcription modulator isoform X10) c4( ) c5(315192) c6( ) c7(T, aC, aCh, pf, bll, bO, fd, bB, ci, aY, bv, aE, Xc, pX, Qk, K, KC, eG, iE, aW, g, as, eF, ir, bw, QA, Qt, iP, eM, iA, aep, m, Xu, ik, n, fr, rP, ft, gd, kH, dL, kj, aq, kp, Qy, SE, dB, ayS, hL, auh, it, B, dA, eR, jx, aA, aNG, lu, q, eA, aT, ahg, rz, ae, jf, s, nO, oV, Y, r, en, aDY, cl, t, Vi, iK, bN, Qi, nM, iM, np, bt, ao, VB, hS, id, LX, bXc, jh, KO, Sv, Fc, aue, ad, dP, av, hd, ib, py, Db, hP, d, bug, je, PE, ff, eH, gl, aF, adj, jb, bz, bF, es, RV, bE, lf, iu); #c1(SLU7) c2(NP_006416) c3(pre-mRNA-splicing factor SLU7) c4(164837) c5(164838, 164839, 164840, 164841, 164842, 164843, 164844, 164845, 164846, 164847, 164848, 164849, 164850, 164851, 164852, 164853, 164854, 164855, 164856, 164857) c6( ) c7(T, bB, v, rP, hF, cd, ad, M, aT, K, bNL, ae, dF, bfl, xx, d, aW, Y, lb, t, nC, bz, bw, bN, iP, W, c, bE, cj, nw, np, m, bt, B, de); #c1(SLU7) c2(XP_005265850) c3(pre-mRNA-splicing factor SLU7 isoform X2) c4(279783, 279784, 279785) c5(279786, 279787, 279788, 279789) c6( ) c7(T, bB, v, rP, hF, cd, ad, M, aT, K, bNL, ae, dF, bfl, xx, d, aW, Y, lb, t, nC, bz, bw, bN, iP, W, c, bE, cj, nw, np, m, bt, B, de); #c1(SLURP1) c2(NP_065160) c3(secreted Ly-6/uPAR-related protein 1 precursor) c4(204341) c5(204342, 204343, 204344, 204345, 204346, 204347) c6( ) c7(T, by, fu, bX, ai, bCx, ld, aT, h, lQ, bU, aW, Y, bhi, baP, lo, dr, Rd, arE, bvc, Lq, hh); #c1(SLX4) c2(NP_115820) c3(structure-specific endonuclease subunit SLX4) c4(224084) c5(224085, 224086, 224087, 224088, 224089, 224090) c6( ) c7(aW, T, os, oZ, aT); #c1(SLX4) c2(XP_011521017) c3(structure-specific endonuclease subunit SLX4 isoform X1) c4( ) c5(315960, 315961, 315962, 315963) c6( ) c7(aW, T, os, oZ, aT); #c1(SLX41P) c2(NP_001009608) c3(protein SLX4IP) c4(23673) c5(23674, 23675, 23676, 23677, 23678, 23679) c6( ) c7(x, k); #c1(SMAD1) c2(NP_005891) c3(mothers against decapentaplegic homolog 1) c4(160138, 160139, 160140, 160141, 160142, 160143) c5(160144, 160145, 160146, 160147, 160148, 160149, 160150, 160151, 160152, 160153, 160154, 160155, 160156, 160157, 160158, 160159, 160160, 160161, 160162) c6( ) c7(aC, gU, if, jh, aA, hy, ah, j, qN, ad, av, aE, BJ, ld, aT, bl, bq, ae, A, B, BN, iU, aW, ff, eH, r, fQ, yy, aq, aF, en, eF, t, Ew, iT, w, hc, m, lC, bt, ne); #c1(SMAD1) c2(XP_005263050) c3(mothers against decapentaplegic homolog 1 isoform X2) c4( ) c5(278247, 278248, 278249, 278250, 278251, 278252, 278253, 278254, 278255, 278256, 278257, 278258, 278259, 278260, 278261, 278262, 278263, 278264, 278265, 278266, 278267, 278268, 278269, 278270, 278271, 278272, 278273, 278274, 278275, 278276) c6( ) c7(aC, gU, if, jh, aA, hy, ah, j, qN, ad, av, aE, BJ, ld, aT, bl, bq, ae, A, B, BN, iU, aW, ff, eH, r, fQ, yy, aq, af, en, eF, t, Ew, iT, w, hc, m, lC, bt, ne); #c1(SMAD2) c2(XP_006722514) c3(mothers against decapentaplegic homolog 2 isoform X1) c4(294721) c5(294722, 294723, 294724, 294725, 294726, 294727, 294728, 294729, 294730, 294731, 294732) c6( ) c7(lD, bM, T, aC, ea, if, al, aHH, fd, bB, aeu, ci, Na, aE, K, bc, ly, Jl, aW, Az, MV, g, eF, akj, eE, cm, eM, iT, vA, Hb, m, NT, aj, fr, qN, BR, bkP, dL, wC, fQ, fe, kp, Hu, op, lx, hk, Vy, mg, at, bD, it, B, fG, yk, jx, aA, v, q, J, eA, brH, sy, aT, cy, ae, yL, r, en, cl, En, t, ik, pb, kP, gX, oX, Ep, bt, hS, jh, ad, av, sN, ab, d, lU, BO, ff, eH, cr, aF, bz, LB, bE, wz, aS, be, MC, lC, ZW); #c1(SMAD2) c2(XP_011524285) c3(mothers against decapentaplegic homolog 2 isoform X2) c4(318774) c5(318775, 318776, 318777, 318778, 318779, 318780) c6( ) c7(lD, bM, T, aC, ea, if, al, aHH, fd, bB, aeu, ci, Na, aE, K, bc, ly, Jl, aW, Az, MV, g, eF, akj, eE, cm, eM, iT, vA, Hb, m, NT, aj, fr, qN, BR, bkP, dL, wC, fQ, fe, kp, Hu, op, lx, hk, Vy, mg, at, bD, it, B, fG, yk, jx, aA, v, q, J, eA, brH, sy, aT, cy, ae, yL, r, en, cl, En, t, iK, pb, kP, gX, oX, Ep, bt, hS, jh, ad, av, sN, ab, d, lU, BO, ff, eH, cr, aF, bz, LB, bE, wz, aS, be, MC, lC, ZW); #c1(SMAD3) c2(NP_001138574) c3(mothers against decapentaplegic homolog 3 isoform 2) c4( ) c5(57024, 57025, 57026, 57027, 57028, 57029) c6( ) c7(lD, bM, T, aC, Ap, fR, ea, al, Av, bB, lR, ci, Na, aE, HR, K, bc, Qe, ly, yo, aW, Az, hl, eE, eM, vA, iS, Hb, m, dD, NT, n, eW, bRT, hw, Do, FN, h, wC, A, fe, aq, kp, op, lx, kT, bP, dB, bo, at, cAe, it, B, bL, dA, fG, yk, jx, aA, hy, v, j, q, eA, mj, BJ, sy, aT, ae, af, jf, afG, bf, NE, Y, yL, la, r, en, t, iK, pb, ck, bnc, iM, ac, dy, bt, ao, vi, eq, hF, Rk, ad, KL, hX, av, F, sN, lC, lz, nJ, d, BN, je, ff, af, iV, bz, bF, es, bE, aS, be, lf, ne, y); #c1(SMAD3) c2(NP_001138575) c3(mothers against decapentaplegic homolog 3 isoform 3) c4(57030) c5(57031, 57032, 57033, 57034, 57035, 57036) c6( ) c7(lD, bM, T, aC, Ap, fR, ea, al, Av, bB, lR, ci, Na, aE, HR, K, bc, Qe, ly, yo, aW, Az, hl, eE, eM, vA, iS, Hb, m, dD, NT, n, eW, bRT, hw, Do, FN, h, wC, A, fe, aq, kp, op, lx, kT, bP, dB, bo, at, cAe, it, B, bL, dA, fG, yk, jx, aA, hy, v, j, q, eA, mj, BJ, sy, aT, ae, af, jf, afG, bf, NE, Y, yL, la, r, en, t, iK, pb, ck, bnc, iM, ac, dy, bt, ao, vi, eq, hF, Rk, ad, KL, hX, av, F, sN, lC, lz, nJ, d, BN, je, ff, aF, iV, bz, bF, es, bE, aS, be, lf, ne, y); #c1(SMAD3) c2(NP_001138576) c3(mothers against decapentaplegic homolog 3 isoform 4) c4( ) c5(57037, 57038, 57039, 57040, 57041) c6( ) c7(lD, bM, T, aC, Ap, fR, ea, al, Av, bB, lR, ci, Na, aE, HR, K, bc, Qe, ly, yo, aW, Az, hl, eE, eM, vA, iS, Hb, m, dD, NT, n, eW, bRT, hw, Do, FN, h, wC, A, fe, aq, kp, op, lx, kT, bP, dB, bo, at, cHe, it, B, bL, dA, fG, yk, jx, aA, hy, v, j, q, eA, mj, BJ, sy, aT, ae, af, jf, afG, bf, NE, Y, yL, la, r, en, t, ik, pb, ck, bnc, iM, ac, dy, bt, ao, vi, eq, hF, Rk, ad, KL, hX, av, F, sN, lC, lz, nJ, d, BN, je, ff, aF, iV, bz, bF, es, bE, aS, be, lf, ne, y); #c1(SMAD3) c2(NP_005893) c3(mothers against decapentaplegic homolog 3 isoform 1) c4(160163, 160164) c5(160165, 160166, 160167, 160168, 160169, 160170, 160171, 160172, 160173, 160174, 160175) c6( ) c7(lD, bM, T, aC, Ap, fR, ea, al, Av, bB, lR, ci, Na, aE, HR, K, bc, Qe, ly, yo, aW, Az, hl, BE, eM, vA, iS, Hb, m, dD, NT, n, eW, bRT, hw, Do, FN, h, wC, A, fe, aq, kp, op, lx, kT, bP, dB, bo, at, cHe, it, B, bL, dA, fG, yk, jx, aA, hy, v, j, q, eA, mj, BJ, sy, aT, ae, af, jf, afG, bf, NE, Y, yL, la, r, en, t, iK, pb, ck, bnc, iM, ac, dy, bt, ao, vi, eq, hF, Rk, ad, KL, hX, av, F, sN, lC, lz, nJ, d, BN, je, ff, aF, iV, bz, bF, es, bE, aS, be, lf, ne, y); #c1(SMAD3) c2(XP_011519861) c3(mothers against decapentaplegic homolog 3 isoform X1) c4(314605) c5( ) c6( ) c7(lD, bM, T, aC, Ap, fR, ea, al, Av, bB, lR, ci, Na, aE, HR, K, bc, Qe, ly, yo, aW, Az, hl, eE, EM, vA, iS, Hb, m, dD, NT, n, eW, bRT, hw, Do, FN, h, wC, A, fe, aq, kp, op, lx, kT, bP, dB, bo, at, cHe, it, B, bL, dA, fG, yk, jx, aA, hy, v, j, q, eA, mj, BJ, sy, aT, ae, af, jf, afG, bf, NE, Y, yL, la, r, en, t, iK, pb, ck, bnc, iM, ac, dy, bt, ao, vi, eq, hF, Rk, ad, kL, hX, av, F, sN, lC, lz, nJ, d, BN, je, ff, aF, iV, bz, bF, es, bE, aS, be, lf, ne, y); #c1(SMAD3) c2(XP_011519862) c3(mothers against decapentaplegic homolog 3 isoform X2) c4( ) c5(314606) c6( ) c7(lD, bM, T, aC, Ap, fR, ea, al, Av, bB, lR, ci, Na, aE, HR, K, bc, Qe, ly, yo, aW, Az, hl, eE, eM, vA, iS, Hb, m, dD, NT, n, eW, bRT, hw, Do, FN, h, wC, A, fe, aq, kp, op, lx, kT, bP, dB, bo, at, cHe, it, B, bL, dA, fG, yk, jx, aA, hy, v, j, q, eA, mj, BJ, sy, aT, ae, af, jf, afG, bf, NE, Y, yL, la, r, en, t, iK, pb, ck, bnc, iM, ac, dy, bt, ao, vi, eq, hF, Rk, ad, KL, hX, av, F, sN, lC, lz, nJ, d, BN, je, ff, aF, iV, bz, bF, es, bE, aS, be, lf, ne, y); #c1(SMAD4) c2(NP_005350) c3(mothers against decapentaplegic homolog 4) c4(155304, 155305, 155306) c5(155307, 155308, 155309, 155310, 155311, 155312, 155313, 155314, 155315, 155316, 155317, 155318, 155319, 155320, 155321, 155322, 155323, 155324, 155325, 155326, 155327, 155328, 155329, 155330) c6( ) c7(T, os, aC, rV, pf, if, al, aHH, bB, lR, ci, ip, Na, bv, aE, gH, auB, K, hu, zy, eL, Us, YL, Jl, aW, Az, nY, g, MV, cm, eM, nB, iA, ki, go, vA, Ji, aep, Hb, m, gn, YK, Qj, wu, sw, ht, NT, n, fr, qN, Cd, eW, Bl, jz, cGT, kl, gd, dL, KV, Yl, wC, Ck, A, aiB, Yd, AQ, ks, fQ, ahJ, aq, kp, op, bP, bcd, it, B, bL, ka, dA, fG, yk, rf, aA, AD, Cb, q, er, eA, eJ, mj, Yn, brH, aT, Tj, ae, kl, blD, Yw, s, vT, Qr, Y, yL, r, en, bu, cl, LV, t, iK, pb, OC, ck, nM, gX, np, bt, bNx, cHf, Mb, oZ, ad, hX, av, hd, Cj, Db, Ot, oj, oW, io, ji, wm, Yi, WW, BO, qt, Hh, ff, cHg, yy, af, iV, bz, bF, es, pc, bE, Lb, aS, MC, lC, X, BA); #c1(SMAD5) c2(NP_001001420) c3(mothers against decapentaplegic homolog 5) c4(18364, 18365, 18366, 18367) c5(18368, 18369, 18370, 18371, 18372, 18373, 18374, 18375, 18376, 18377, 18378, 18379, 18380, 18381, 18382, 18383, 18384, 18385, 18386, 18387, 18388, 18389, 18390, 18391, 18392, 18393, 18394, 18395, 18396) c6( ) c7(ck, av, T, A, bP, fR, go, BJ, lC, r, yy, hN, fe, bw, ji, q, iV, akj, bt, B); #c1(SMAD6) c2(NP_005576) c3(mothers against decapentaplegic homolog 6) c4(157592, 157593) c5(157594, 157595, 157596, 157597, 157598, 157599, 157600, 157601, 157602, 157603, 157604, 157605, 157606) c6( ) c7(bL, adf, acW, eM, ff, lC, r, at, si, ci, K, m, uE, t, en, bz, q, eA); #c1(SMAD6) c2(XP_011519863) c3(mothers against decapentaplegic homolog 6 isoform X1) c4(314607) c5(314608, 314609, 314610, 314611, 314612, 314613) c6( ) c7(bL, adf, acW, eM, ff, lC, r, at, si, ci, K, m, uE, t, en, bz, q, eA); #c1(SMAD7) c2(NP_001177751) c3(mothers against decapentaplegic homolog 7 isoform 3) c4( ) c5(72961, 72962, 72963, 72964, 72965, 72966, 72967, 72968) c6( ) c7(bL, bM, T, aC, dA, wu, sw, fG, acl, aHH, fZ, aA, n, hy, v, j, ci, uE, bB, qN, lR, fr, q, ad, eA, Na, blC, aE, il, aT, vw, je, ji, K, wm, pj, Ha, ae, aiJ, Qe, cHh, kl, B, BN, yo, vT, A, aW, Y, yL, az, gC, oV, r, fe, ff, kp, af, en, cl, t, bw, bz, pb, op, ck, es, il, iM, nB, bE, w, ag, vA, cEp, X, hL, cj, Hs, dy, m, byK, ne); #c1(SMAD7) c2(NP_005895) c3(mothers against decapentaplegic homolog 7 isoform 1) c4(160176, 160177) c5(160178, 160179, 160180, 160181, 160182, 160183, 160184, 160185, 160186) c6( ) c7(bL, bM, T, aC, dA, wu, sw, fG, acl, aHH, fZ, aA, n, hy, v, j, ci, uE, bB, qN, lR, fr, q, ad, eA, Na, blC, aE, il, aT, vw, je, ji, K, wm, pj, Ha, ae, aiJ, Qe, cHh, kl, B, BN, yo, vT, A, aW, Y, yL, az, gC, oV, r, fe, ff, kp, aF, en, cl, t, bw, bz, pb, op, ck, es, iO, iM, nB, bE, w, ag, vA, cEp, X, hL, cj, Hs, dy, m, byK, ne); #c1(SMAD9) c2(NP_001120689) c3(mothers against decapentaplegic homolog 9 isoform a) c4(44592) c5(44593, 44594, 44595, 44596, 44597, 44598, 44599, 44600, 44601, 44602) c6( ) c7(lD, ly, T, s, cHi, bB, BZ, lC, ae, ad, lx, BY); #c1(SMAD9) c2(NP_005896) c3(mothers against decapentaplegic homolog 9 isoform b) c4(160187) c5(160188, 160189, 160190, 160191, 160192, 160193, 160194) c6( ) c7(lD, ly, T, s, cHi, bB, BZ, lC, ae, ad, lx, BY); #c1(SMAGP) c2(NP_001029045) c3(small cell adhesion glycoprotein) c4(30743) c5(30744, 30745, 30746, 30747, 30748, 30749, 30750, 30751, 30752, 30753) c6( ) c7(T, q, eJ, r); #c1(SMAP1) c2(NP_001037770) c3(stromal membrane-associated protein 1 isoform A) c4(34128, 34129) c5(34130, 34131, 34132, 34133, 34134, 34135) c6( ) c7(r); #c1(SMAP1) c2(NP_001268368) c3(stromal membrane-associated protein 1 isoform C) c4(99796) c5(99797, 99798, 99799, 99800, 99801, 99802) c6( ) c7(r); #c1(SMAP1) c2(NP_068759) c3(stromal membrane-associated protein 1 isoform B) c4(210243) c5(210244, 210245, 210246, 210247, 210248, 210249) c6( ) c7(r); #c1(SMAP1) c2(XP_005248817) c3(stromal membrane-associated protein 1 isoform X5) c4(268611, 268612) c5(268613, 268614, 268615, 268616, 268617, 268618, 268619, 268620, 268621, 268622, 268623, 268624, 268625, 268626, 268627, 268628, 268629, 268630, 268631, 268632, 268633, 268634, 268635, 268636) c6( ) c7(r); #c1(SMAP1) c2(XP_011534350) c3(stromal membrane-associated protein 1 isoform X4) c4(330730) c5( ) c6( ) c7(r); #c1(SMARCA1) c2(NP_001269803) c3(probable global transcription activator SNF2L1 isoform d) c4(102879) c5( ) c6( ) c7(eW, T, Or, bB, Sv, iJ, q, ad, eA, hX, ip, bv, av, x, py, aE, cHj, agk, ae, gj, d, Y, ff, r, nf, lo, La, en, as, t, bz, ck, es, bP, eM, iA, bE, k, B); #c1(SMARCA1) c2(XP_005262518) c3(probable global transcription activator SNF2L1 isoform X1) c4(277992) c5(277993, 277994, 277995, 277996, 277997, 277998, 277999) c6( ) c7(eW, T, Or, bB, Sv, iJ, q, ad, eA, hX, ip, bv, av, x, py, aE, cHj, agk, ae, gj, d, Y, ff, r, nf, lo, La, en, as, t, bz, ck, es, bP, eM, iA, bE, k, B); #c1(SMARCA1) c2(XP_005262519) c3(probable global transcription activator SNF2L1 isoform X2) c4( ) c5(278000, 278001, 278002, 278003, 278004, 278005) c6( ) c7(eW, T, Or, bB, Sv, iJ, q, ad, eA, hX, ip, bv, av, x, py, aE, cHj, agk, ae, gj, d, Y, ff, r, nf, lo, La, en, as, t, bz, ck, es, bP, eM, iA, bE, k, B); #c1(SMARCA1) c2(XP_006724845) c3(probable global transcription activator SNF2L1 isoform X3) c4(296453) c5(296454, 296455, 296456, 296457, 296458, 296459, 296460, 296461, 296462, 296463) c6( ) c7(eW, T, Or, bB, Sv, iJ, q, ad, eA, hX, ip, bv, av, x, py, aE, cHj, agk, ae, gj, d, Y, ff, r, nf, lo, La, en, as, t, bz, ck, es, bP, eM, iA, bE, k, B); #c1(SMARCA2) c2(NP_001276326) c3(probable global transcription activator SNF2L2 isoform c) c4(108032, 108033) c5(108034, 108035, 108036, 108037, 108038, 108039, 108040, 108041, 108042, 108043, 108044, 108045, 108046) c6( ) c7(T, yk, Dr, aw, EQ, v, bRy, Ea, ci, Fc, id, eA, Pu, ad, eJ, av, iu, aE, cHk, qK, aT, agk, rz, d, Y, ff, nf, La, en, g, as, ik, all, ck, sS, ac, qe, ER, B); #c1(SMARCA2) c2(NP_001276327) c3(probable global transcription activator SNF2L2 isoform d) c4(108047) c5(108048, 108049, 108050, 108051, 108052, 108053, 108054, 108055, 108056, 108057, 108058, 108059, 108060, 108061, 108062, 108063, 108064, 108065, 108066, 108067, 108068, 108069, 108070, 108071) c6( ) c7(T, yk, Or, aw, EQ, v, bRy, Ea, ci, Fc, iJ, eA, Pu, ad, eJ, av, iu, aE, cHk, qK, aT, agk, rz, d, Y, ff, nf, La, en, g, as, ik, all, ck, sS, ac, qe, ER, B); #c1(SMARCA2) c2(NP_001276328) c3(probable global transcription activator SNF2L2 isoform e precursor) c4(108072, 108073) c5(108074, 108075, 108076, 108077, 108078, 108079, 108080, 108081, 108082, 108083, 108084) c6( ) c7(T, yk, Or, aw, EQ, v, bRy, Ea, ci, Fc, iJ, eA, Pu, ad, eJ, av, iu, aE, cHk, qK, aT, agk, rz, d, Y, ff, nf, La, en, g, as, ik, all, ck, sS, ac, qe, ER, B); #c1(SMARCA2) c2(NP_001276329) c3(probable global transcription activator SNF2L2 isoform f precursor) c4(108085) c5(108086, 108087, 108088, 108089, 108090, 108091, 108092, 108093) c6( ) c7(T, yk, Or, aw, EQ, v, bRy, Ea, ci, Fc, iJ, eA, Pu, ad, eJ, av, iu, aE, cHk, qK, aT, agk, rz, d, Y, ff, nf, La, en, g, as, iK, all, ck, sS, ac, qe, ER, B); #c1(SMARCA2) c2(NP_003061) c3(probable global transcription activator SNF2L2 isoform a) c4(134822, 134823, 134824, 134825, 134826) c5(134827, 134828, 134829, 134830, 134831, 134832, 134833, 134834, 134835, 134836, 134837, 134838, 134839, 134840, 134841, 134842, 134843, 134844, 134845, 134846, 134847, 134848, 134849) c6( ) c7(T, yk, Or, aw, EQ, v, bRy, Ea, ci, Fc, iJ, eA, Pu, ad, eJ, av, iu, aE, cHk, qK, aT, agk, rz, d, Y, ff, nf, La, en, g, as, iK, all, ck, sS, ac, qe, ER, B); #c1(SMARCA2) c2(NP_620614) c3(probable global transcription activator SNF2L2 isoform b) c4(237998, 237999) c5(238000, 238001, 238002, 238003, 238004, 238005, 238006, 238007, 238008, 238009, 238010) c6( ) c7(T, yk, Or, aw, EQ, v, bRy, Ea, ci, Fc, iJ, eA, Pu, ad, eJ, av, iu, aE, cHk, qK, aT, agk, rz, d, Y, ff, nf, La, en, g, as, ik, all, ck, sS, ac, qe, ER, B); #c1(SMARCA4) c2(NP_001122316) c3(transcription activator BRG1 isoform B) c4(46516) c5(46517, 46518, 46519, 46520, 46521, 46522, 46523, 46524, 46525, 46526, 46527) c6( ) c7(T, os, dD, ea, yk, e, Or, aw, iW, aA, jh, oZ, j, ci, eq, Fc, q, Pu, ad, qH, eA, bv, eJ, ft, av, aE, ec, iu, x, oj, aT, vg, vc, K, bq, agk, pk, ae, jf, cHm, d, je, s, cHl, aW, Y, AQ, cHn, ff, nf, aF, La, en, g, as, t, bz, all, ck, sS, iM, ac, qe, asl, bE, eM, bP, OD, k, ER, MC, lC, B); #c1(SMARCA4) c2(XP_005260085) c3(transcription activator BRG1 isoform X7) c4(276435) c5( ) c6( ) c7(T, os, dD, ea, yk, e, Or, aw, iW, aA, jh, oZ, j, ci, eq, Fc, q, Pu, ad, qH, eA, bv, eJ, ft, av, aE, ec, iu, x, oj, aT, vg, vc, K, bq, agk, pk, ae, jf, cHm, d, je, s, cHl, aW, Y, AQ, cHn, ff, nf, aF, La, en, g, as, t, bz, all, ck, sS, iM, ac, qe, asl, bE, eM, bP, OD, k, ER, MC, lC, B); #c1(SMARCA4) c2(XP_005260089) c3(transcription activator BRG1 isoform X1) c4(276436) c5(276437, 276438, 276439, 276440, 276441, 276442, 276443, 276444, 276445, 276446, 276447, 276448) c6( ) c7(T, os, dD, ea, yk, e, Or, aw, iW, aA, jh, oZ, j, ci, eq, Fc, q, Pu, ad, qH, eA, bv, eJ, ft, av, aE, ec, iu, x, oj, aT, vg, vc, K, bq, agk, pk, ae, jf, cHm, d, je, s, cHl, aW, Y, AQ, cHn, ff, nf, aF, La, en, g, as, t, bz, all, ck, sS, iM, ac, qe, asl, bE, eM, bP, OD, k, ER, MC, lC, B); #c1(SMARCA4) c2(XP_005260090) c3(transcription activator BRG1 isoform X2) c4(276449) c5(276450, 276451, 276452, 276453, 276454, 276455) c6( ) c7(T, os, dD, ea, yk, e, Or, aw, iW, aA, jh, oZ, j, ci, eq, Fc, q, Pu, ad, qH, eA, bv, eJ, ft, av, aE, ec, iu, x, oj, aT, vg, vc, K, bq, agk, pk, ae, jf, cHm, d, je, s, cHl, aW, Y, AQ, cHn, ff, nf, aF, La, en, g, as, t, bz, all, ck, sS, iM, ac, qe, asl, bE, eM, bP, OD, k, ER, MC, lC, B); #c1(SMARCA4) c2(XP_005260091) c3(transcription activator BRG1 isoform X3) c4(276456) c5(276457, 276458, 276459, 276460, 276461, 276462) c6( ) c7(T, os, dD, ea, yk, e, Or, aw, iW, aA, jh, oZ, j, ci, eq, Fc, q, Pu, ad, qH, eA, bv, eJ, ft, av, aE, ec, iu, x, oj, aT, vg, vc, K, bq, agk, pk, ae, jf, cHm, d, je, s, CHl, aW, Y, AQ, cHn, ff, nf, af, La, en, g, as, t, bz, all, ck, sS, iM, ac, qe, asl, bE, eM, bP, OD, k, ER, MC, lC, B); #c1(SMARCA4) c2(XP_005260092) c3(transcription activator BRG1 isoform X4) c4(276463) c5(276464, 276465, 276466, 276467, 276468, 276469, 276470, 276471, 276472, 276473, 276474, 276475, 276476, 276477, 276478) c6( ) c7(T, os, dD, ea, yk, e, Or, aw, iW, aA, jh, oZ, j, ci, eq, Fc, q, Pu, ad, qH, eA, bv, eJ, ft, av, aE, ec, iu, x, oj, aT, vg, vc, K, bq, agk, pk, ae, jf, cHm, d, je, s, cHl, aW, Y, AQ, cHn, ff, nf, aF, La, en, g, as, t, bz, all, ck, sS, iM, ac, qe, asl, bE, eM, bP, OD, k, ER, MC, lC, B); #c1(SMARCA4) c2(XP_011526500) c3(transcription activator BRG1 isoform X5) c4(320791) c5(320792, 320793, 320794, 320795, 320796, 320797) c6( ) c7(T, os, dD, ea, yk, e, Or, aw, iW, aA, jh, oZ, j, ci, eq, Fc, q, Pu, ad, qH, eA, bv, eJ, ft, av, aE, ec, iu, x, oj, aT, vg, vc, K, bq, agk, pk, ae, jf, cHm, d, je, s, cHl, aW, Y, AQ, cHn, ff, nf, aF, La, en, g, as, t, bz, all, ck, sS, iM, ac, qe, asl, bE, eM, bP, OD, k, ER, MC, lC, B); #c1(SMARCA5) c2(NP_003592) c3(SWI/SNF-related matrix-associated actin-dependent regulator of chromatin subfamily A member 5) c4(139418, 139419) c5(139420, 139421, 139422, 139423, 139424, 139425, 139426, 139427, 139428, 139429, 139430, 139431, 139432, 139433, 139434, 139435) c6( ) c7(ck, iF, jf, aW, aT, Ou, S, hF, bt, eW, dx, hX); #c1(SMARCAD1) c2(NP_001121901) c3(SWI/SNF-related matrix-associated actin-dependent regulator of chromatin subfamily A containing DEAD/H box 1 isoform a) c4(46316, 46317) c5(46318, 46319, 46320, 46321, 46322, 46323, 46324, 46325, 46326, 46327) c6( ) c7(bL, T, hW, LX, eH, eF, he); #c1(SMARCAD1) c2(NP_064544) c3(SWI/SNF-related matrix-associated actin-dependent regulator of chromatin subfamily A containing DEAD/H box 1 isoform b) c4(202913) c5(202914, 202915, 202916, 202917, 202918, 202919, 202920, 202921, 202922, 202923, 202924, 202925) c6( ) c7(bL, T, hW, LX, eH, eF, he); #c1(SMARCAL1) c2(XP_005246688) c3(SWI/SNF-related matrix-associated actin-dependent regulator of chromatin subfamily A-like protein 1 isoform X1) c4(267342) c5(267343, 267344, 267345, 267346, 267347, 267348, 267349, 267350, 267351, 267352, 267353, 267354, 267355, 267356, 267357, 267358, 267359) c6( ) c7(Pw, Pv, aGC, eR, gi, dy, K, lM, hE, Pu); #c1(SMARCAL1) c2(XP_006712620) c3(SWI/SNF-related matrix-associated actin-dependent regulator of chromatin subfamily A-like protein 1 isoform X2) c4(286746, 286747, 286748, 286749) c5(286750, 286751, 286752, 286753, 286754, 286755, 286756, 286757, 286758, 286759, 286760, 286761, 286762, 286763, 286764, 286765, 286766, 286767, 286768) c6( ) c7(Pw, Pv, aGC, eR, gi, dy, K, lM, hE, Pu); #c1(SMARCB1) c2(NP_001007469) c3(SWI/SNF-related matrix-associated actin-dependent regulator of chromatin subfamily B member 1 isoform b) c4(22727) c5(22728, 22729, 22730, 22731, 22732, 22733, 22734) c6( ) c7(T, kh, aC, aiH, fR, YU, aNA, Or, n, Sv, ci, cHs, Fc, anu, q, er, qH, eA, ip, bv, x, byh, lg, byg, jZ, SV, aT, dV, MU, Ou, bnt, cHq, K, aDC, hP, agk, h, dx, cp, jf, if, eG, Uf, d, iU, Hq, Y, Ls, cHn, ev, ff, r, cHt, Am, dg, aiz, en, g, kJ, cm, bz, Qt, op, Vi, Ax, Hp, es, QG, Ew, bP, dB, dc, vA, AH, RW, cHr, cHu, cHo, k, cHp, bt, jr, ayg); #c1(SMARCB1) c2(NP_003064) c3(SWI/SNF-related matrix-associated actin-dependent regulator of chromatin subfamily B member 1 isoform a) c4(134873, 134874) c5(134875, 134876, 134877, 134878, 134879, 134880, 134881) c6( ) c7(T, kh, aC, aiH, fR, YU, aNA, Or, n, Sv, ci, cHs, Fc, anu, q, er, qH, eA, ip, bv, x, byh, lg, byg, jZ, SV, aT, dV, MU, Ou, bnt, cHq, K, aDC, hP, agk, h, dx, cp, jf, if, eG, Uf, d, iU, Hq, Y, Ls, cHn, ev, ff, r, cHt, Am, dg, aiz, en, g, kJ, cm, bz, Qt, op, Vi, Qx, Hp, es, QG, Ew, bP, dB, dc, vA, AH, RW, cHr, cHu, cHo, k, cHp, bt, jr, ayg); #c1(SMARCB1) c2(XP_011545210) c3(SWI/SNF-related matrix-associated actin-dependent regulator of chromatin subfamily B member 1 isoform X1) c4( ) c5(343995, 343996, 343997, 343998, 343999) c6( ) c7(T, kh, aC, aiH, fR, YU, aNA, Or, n, Sv, ci, cHs, Fc, anu, q, er, qH, eA, ip, bv, x, byh, lg, byg, jZ, SV, aT, dV, MU, Ou, bnt, cHq, K, aDC, hP, agk, h, dx, cp, jf, iF, eG, Uf, d, iU, Hq, Y, Ls, cHn, ev, ff, r, cHt, Am, dg, aiz, en, g, kJ, cm, bz, Qt, op, Vi, Qx, Hp, es, QG, Ew, bP, dB, dc, vA, AH, RW, cHr, cHu, cHo, k, cHp, bt, jr, ayg); #c1(SMARCC1) c2(NP_003065) c3(SWI/SNF complex subunit SMARCC1) c4(134882) c5(134883, 134884, 134885, 134886, 134887, 134888, 134889, 134890) c6( ) c7(av, aE, aW, Dd, r, aT, n, K, g, q, ad); #c1(SMARCC1) c2(XP_011532336) c3(SWI/SNF complex subunit SMARCC1 isoform X1) c4( ) c5(328485, 328486, 328487) c6( ) c7(av, aE, aW, Dd, r, aT, n, K, g, q, ad); #c1(SMARCC1) c2(XP_011532337) c3(SWI/SNF complex subunit SMARCC1 isoform X2) c4( ) c5(328488) c6( ) c7(av, aE, aW, Dd, r, aT, n, K, g, q, ad); #c1(SMARCC2) c2(NP_001123892) c3(SWI/SNF complex subunit SMARCC2 isoform c) c4(47982) c5( ) c6( ) c7(T, EY, K); #c1(SMARCC2) c2(NP_003066) c3(SWI/SNF complex subunit SMARCC2 isoform a) c4(134891) c5(134892, 134893, 134894, 134895, 134896, 134897, 134898) c6( ) c7(T, EY, K); #c1(SMARCC2) c2(NP_620706) c3(SWI/SNF complex subunit SMARCC2 isoform b) c4(238065) c5( ) c6( ) c7(T, EY, K); #c1(SMARCC2) c2(XP_005269158) c3(SWI/SNF complex subunit SMARCC2 isoform X1) c4( ) c5(282364, 282365, 282366, 282367, 282368, 282369, 282370, 282371, 282372, 282373, 282374, 282375) c6( ) c7(T, EY, K); #c1(SMARCD1) c2(NP_003067) c3(SWI/SNF-related matrix-associated actin-dependent regulator of chromatin subfamily D member 1 isoform a) c4(134899, 134900) c5(134901, 134902, 134903, 134904, 134905, 134906) c6( ) c7(T, cHv); #c1(SMARCD1) c2(NP_620710) c3(SWI/SNF-related matrix-associated actin-dependent regulator of chromatin subfamily D member 1 isoform b) c4(238090) c5(238091, 238092, 238093, 238094, 238095, 238096) c6( ) c7(T, cHv); #c1(SMARCD1) c2(XP_005269164) c3(SWI/SNF-related matrix-associated actin-dependent regulator of chromatin subfamily D member 1 isoform X1) c4( ) c5(282376, 282377, 282378, 282379, 282380, 282381) c6( ) c7(T, cHv); #c1(SMARCD3) c2(NP_001003801) c3(SWI/SNF-related matrix-associated actin-dependent regulator of chromatin subfamily D member 3 isoform 2) c4(20155) c5(20156, 20157, 20158, 20159, 20160, 20161) c6( ) c7(aAx, kP); #c1(SMARCD3) c2(NP_003069) c3(SWI/SNF-related matrix-associated actin-dependent regulator of chromatin subfamily D member 3 isoform 1) c4(134907, 134908, 134909) c5(134910, 134911, 134912, 134913, 134914, 134915, 134916, 134917, 134918, 134919, 134920, 134921, 134922, 134923, 134924, 134925) c6( ) c7(aAx, kP); #c1(SMARCD3) c2(XP_011514823) c3(SWI/SNF-related matrix-associated actin-dependent regulator of chromatin subfamily D member 3 isoform X1) c4( ) c5(307505, 307506, 307507, 307508, 307509, 307510) c6( ) c7(aAx, KP); #c1(SMARCE1) c2(NP_003070) c3(SWI/SNF-related matrix-associated actin-dependent regulator of chromatin subfamily E member 1) c4(134926, 134927, 134928, 134929, 134930, 134931, 134932) c5(134933, 134934, 134935, 134936, 134937, 134938, 134939, 134940, 134941, 134942, 134943, 134944, 134945, 134946, 134947, 134948, 134949, 134950, 134951, 134952, 134953, 134954, 134955, 134956, 134957, 134958, 134959, 134960, 134961, 134962, 134963, 134964, 134965, 134966, 134967, 134968, 134969, 134970, 134971, 134972, 134973, 134974, 134975) c6( ) c7(aiB, aW, qK, aT, cHj, agk, cfu, ae, ad); #c1(SMC1A) c2(NP_001268392) c3(structural maintenance of chromosomes protein 1A isoform 2) c4(99826, 99827) c5(99828, 99829, 99830, 99831, 99832, 99833, 99834, 99835, 99836, 99837, 99838, 99839) c6( ) c7(kP, bei, iT, aW, qK, eZ, aT, r, bWO, aNL, dL, aDC, nA, bC, nC); #c1(SMC1A) c2(NP_006297) c3(structural maintenance of chromosomes protein 1A isoform 1) c4(163788, 163789) c5(163790, 163791, 163792, 163793, 163794, 163795, 163796, 163797, 163798, 163799, 163800, 163801) c6( ) c7(kP, bei, iT, aW, qK, eZ, aT, r, bWO, aNL, dL, aDC, nA, bC, nC); #c1(SMC1B) c2(NP_001278430) c3(structural maintenance of chromosomes protein 18 isoform 2) c4(109749) c5(109750, 109751, 109752, 109753, 109754, 109755) c6( ) c7(BN, ND, vH, hy, dL, ne); #c1(SMC1B) c2(NP_683515) c3(structural maintenance of chromosomes protein 1B isoform 1) c4(242362) c5(242363, 242364, 242365, 242366, 242367, 242368) c6( ) c7(BN, ND, vH, hy, dL, ne); #c1(SMC2) c2(XP_011516451) c3(structural maintenance of chromosomes protein 2 isoform X1) c4(309733, 309734) c5(309735, 309736, 309737, 309738, 309739, 309740, 309741, 309742, 309743, 309744, 309745, 309746, 309747, 309748) c6( ) c7(ck, dA, jh, gl, cjN, bX, Eg, fq, de); #c1(SMC2) c2(XP_011516453) c3(structural maintenance of chromosomes protein 2 isoform X2) c4( ) c5(309749) c6( ) c7(ck, dA, jh, gl, cjN, bX, Eg, fq, Qe); #c1(SMC2) c2(XP_011516455) c3(structural maintenance of chromosomes protein 2 isoform X4) c4(309750, 309751) c5(309752, 309753, 309754, 309755, 309756, 309757) c6( ) c7(ck, dA, jh, gl, cjN, bX, Eg, fq, Qe); #c1(SMC2) c2(XP_011516456) c3(structural maintenance of chromosomes protein 2 isoform X5) c4( ) c5(309758, 309759, 309760, 309761, 309762, 309763, 309764, 309765, 309766, 309767, 309768) c6( ) c7(ck, dA, jh, gl, cjN, bX, Eg, fq, Qe); #c1(SMC3) c2(NP_005436) c3(structural maintenance of chromosomes protein 3) c4(156162) c5(156163, 156164, 156165, 156166, 156167, 156168) c6( ) c7(T, cHw, bei, Yc, iT, aeB, vJ, bE, aNL, qG, ci, nA, g, nC); #c1(SMC4) c2(NP_005487) c3(structural maintenance of chromosomes protein 4 isoform 1) c4(156712, 156713, 156714, 156715, 156716, 156717, 156718) c5(156719, 156720, 156721, 156722, 156723, 156724, 156725, 156726, 156727, 156728, 156729, 156730, 156731, 156732, 156733, 156734, 156735, 156736, 156737, 156738, 156739, 156740, 156741, 156742, 156743, 156744) c6( ) c7(T, aW, jh, aT, cjN, ey, ae, eA, ad); #c1(SMC4) c2(XP_006713522) c3(structural maintenance of chromosomes protein 4 isoform X3) c4( ) c5(287188, 287189, 287190, 287191, 287192, 287193, 287194, 287195, 287196, 287197, 287198, 287199, 287200, 287201, 287202, 287203, 287204, 287205, 287206, 287207, 287208, 287209, 287210, 287211, 287212, 287213, 287214, 287215, 287216, 287217, 287218, 287219, 287220, 287221) c6( ) c7(T, aW, jh, aT, cjN, ey, ae, eA, ad); #c1(SMC4) c2(XP_011510615) c3(structural maintenance of chromosomes protein 4 isoform X4) c4(301074) c5( ) c6( ) c7(T, aW, jh, aT, cjN, ey, ae, eA, ad); #c1(SMC5) c2(NP_055925) c3(structural maintenance of chromosomes protein 5) c4(184483) c5(184484, 184485, 184486, 184487, 184488, 184489) c6( ) c7(fq, ad); #c1(SMC6) c2(NP_001135758) c3(structural maintenance of chromosomes protein 6) c4(53109, 53110) c5(53111, 53112, 53113, 53114, 53115, 53116, 53117, 53118, 53119, 53120, 53121, 53122, 53123, 53124, 53125, 53126, 53127, 53128, 53129, 53130) c6( ) c7(aW, aT); #c1(SMC6) c2(XP_011531409) c3(structural maintenance of chromosomes protein 6 isoform X1) c4(327493) c5(327494, 327495, 327496, 327497, 327498) c6( ) c7(aW, aT); #c1(SMC6) c2(XP_011531410) c3(structural maintenance of chromosomes protein 6 isoform X2) c4( ) c5(327499) c6( ) c7(aW, aT); #c1(SMCHD1) c2(NP_056110) c3(structural maintenance of chromosomes flexible hinge domain-containing protein 1) c4(185756, 185757) c5(185758, 185759, 185760, 185761, 185762, 185763, 185764, 185765, 185766, 185767, 185768, 185769) c6( ) c7(T, oV, cHx, jh, rx, ey, hf, fq); #c1(SMC04) c2(NP_064564) c3(single-pass membrane and coiled-coil domain-containing protein 4) c4(202990, 202991, 202992) c5(202993, 202994, 202995, 202996) c6( ) c7(cd); #c1(SMC04) c2(XP_011541211) c3(single-pass membrane and coiled-coil domain-containing protein 4 isoform X3) c4( ) c5(339836, 339837, 339838) c6( ) c7(cd); #c1(SMC04) c2(XP_011541213) c3(single-pass membrane and coiled-coil domain-containing protein 4 isoform X5) c4( ) c5(339839, 339840, 339841, 339842, 339843, 339844, 339845, 339846, 339847, 339848, 339849, 339850, 339851, 339852, 339853) c6( ) c7(cd); #c1(SMCP) c2(NP_109588) c3(sperm mitochondrial-associated cysteine-rich protein) c4(219653) c5(219654, 219655, 219656, 219657, 219658, 219659, 219660) c6( ) c7(x, cc, bE, aW, Y, arw, m, k, t, Bu, fq, mu); #c1(SMEK1) c2(NP_001271209) c3(serine/threonine-protein phosphatase 4 regulatory subunit 3A isoform 1) c4(103612) c5(103613, 103614, 103615, 103616, 103617, 103618, 103619, 103620, 103621, 103622) c6( ) c7(cd); #c1(SMEK1) c2(NP_001271210) c3(serine/threonine-protein phosphatase 4 regulatory subunit 3A isoform 3) c4(103623) c5(103624, 103625, 103626, 103627, 103628, 103629, 103630, 103631, 103632, 103633, 103634) c6( ) c7(cd); #c1(SMEK1) c2(XP_005267899) c3(serine/threonine-protein phosphatase 4 regulatory subunit 3A isoform X1) c4(281281, 281282) c5(281283, 281284, 281285, 281286, 281287, 281288) c6( ) c7(cd); #c1(SMEK1) c2(XP_011535238) c3(serine/threonine-protein phosphatase 4 regulatory subunit 3A isoform X2) c4(332088, 332089) c5(332090, 332091, 332092, 332093, 332094, 332095, 332096, 332097, 332098, 332099, 332100, 332101, 332102, 332103, 332104, 332105, 332106, 332107) c6( ) c7(cd); #c1(SMEK2) c2(NP_001116436) c3(serine/threonine-protein phosphatase 4 regulatory subunit 38 isoform 1) c4(43882) c5(43883, 43884, 43885, 43886, 43887, 43888, 43889) c6( ) c7(cH, om, oe); #c1(SMEK2) c2(NP_065196) c3(serine/threonine-protein phosphatase 4 regulatory subunit 3B isoform 2) c4(204547) c5(204548, 204549, 204550, 204551, 204552, 204553) c6( ) c7(cH, om, oe); #c1(SMG1) c2(NP_055907) c3(serine/threonine-protein kinase SMG1) c4(184408, 184409) c5(184410, 184411, 184412, 184413, 184414, 184415, 184416, 184417, 184418, 184419, 184420, 184421) c6( ) c7(T, DD, fd, cR, fr, ci, ai, Vp, hd, aE, aT, h, K, ak, aW, rU, anW, cm, ik, LB, ck, qJ, y, bt, B); #c1(SMG1) c2(XP_005255240) c3(serine/threonine-protein kinase SMG1 isoform X3) c4(273572) c5(273573, 273574, 273575, 273576, 273577, 273578) c6( ) c7(T, DD, fd, cR, fr, ci, ai, Vp, hd, aE, aT, h, K, ak, aW, rU, anW, cm, ik, LB, ck, qJ, y, bt, B); #c1(SMG1) c2(XP_011544072) c3(serine/threonine-protein kinase SMG1 isoform X5) c4( ) c5(343250) c6( ) c7(T, DD, fd, cR, fr, ci, ai, Vp, hd, aE, aT, h, K, ak, aW, rU, anW, cm, ik, LB, ck, qJ, y, bt, B); #c1(SMG6) c2(NP_001269255) c3 (telomerase-binding protein EST1A isoform 4) c4(100917) c5(100918, 100919, 100920, 100921, 100922, 100923) c6( ) c7(e, j); #c1(SMG6) c2(NP_060045) c3(telomerase-binding protein EST1A isoform 1) c4(193861, 193862, 193863, 193864) c5(193865, 193866, 193867, 193868, 193869, 193870, 193871, 193872, 193873, 193874, 193875, 193876) c6( ) c7(e, j); #c1(SMG6) c2(XP_005256628) c3(telomerase-binding protein EST1A isoform X1) c4( ) c5(274261, 274262, 274263, 274264, 274265, 274266) c6( ) c7(e, j); #c1(SMG8) c2(NP_060619) c3(protein SMG8) c4(196847, 196848) c5(196849, 196850, 196851, 196852, 196853, 196854, 196855, 196856, 196857, 196858, 196859, 196860) c6( ) c7(dB, fs); #c1(SMIM15) c2(NP_001041714) c3(small integral membrane protein 15) c4(34320) c5(34321, 34322, 34323, 34324, 34325, 34326, 34327, 34328, 34329, 34330) c6( ) c7(oe); #c1(SMIM15) c2(NP_001129148) c3(small integral membrane protein 19) c4(50919) c5(50920, 50921, 50922, 50923, 50924, 50925, 50926, 50927, 50928, 50929, 50930, 50931, 50932, 50933, 50934, 50935, 50936, 50937) c6( ) c7(oe, pN); #c1(SMIM19) c2(XP_005273455) c3(small integral membrane protein 19 isoform X1) c4( ) c5(284595, 284596, 284597, 284598) c6( ) c7(oe, pN); #c1(SMIM20) c2(NP_001138904) c3(small integral membrane protein 20) c4(58116, 58117) c5(58118, 58119, 58120, 58121, 58122, 58123, 58124, 58125, 58126, 58127, 58128, 58129) c6( ) c7(y, A); #c1(SMIM21) c2(NP_001032408) c3(small integral membrane protein 21 isoform 1) c4(31310) c5(31311, 31312, 31313, 31314, 31315, 31316, 31317, 31318, 31319, 31320) c6( ) c7(ac, oe, bK, dy, fq, bA); #c1(SMIM23) c2(NP_001276899) c3(small integral membrane protein 23) c4(108583) c5(108584, 108585, 108586, 108587, 108588, 108589) c6( ) c7(sj); #c1(SMIM5) c2(XP_011523418) c3(small integral membrane protein 5 isoform X2) c4( ) c5(317943) c6( ) c7(py); #c1(SMIM5) c2(XP_011523421) c3(small integral membrane protein 5 isoform X1) c4(317944) c5(317945, 317946, 317947, 317948, 317949) c6( ) c7(py); #c1(SMN2) c2(NP_059107) c3(survival motor neuron protein isoform d) c4(193043) c5(193044, 193045, 193046, 193047, 193048, 193049, 193050, 193051, 193052, 193053, 193054) c6( ) c7(bM, T, boQ, aey, agU, Vd, ci, arX, x, K, bU, ass, apl, Hl, aW, MV, cm, aoS, bdx, ly, eM, cHz, aem, U, cHy, k, Ro, Ol, n, fr, b, no, Ak, bl, Ha, mJ, dx, Ad, Ff, gY, axV, oh, MW, bD, it, B, fG, aA, eA, ahL, aT, kf, rz, bsd, aTm, hM, aEk, aYp, pb, beH, beJ, apq, c, cHA, bt, VB, Pt, acl, wV, fu, aoz, hd, afm, oj, biX, Oq, xs, eZ, af, Pd, dr, aeb, LB, axl, RQ, Hs, i); #c1(SMN2) c2(NP_075013) c3(survival motor neuron protein isoform a) c4(213504) c5(213505, 213506, 213507, 213508, 213509, 213510, 213511, 213512, 213513, 213514, 213515) c6( ) c7(bM, T, boQ, aey, agU, Vd, ci, arX, x, K, bU, ass, apl, Hl, aW, MV, cm, aoS, bdx, ly, eM, cHz, aem, U, cHy, k, Ro, Ol, n, fr, b, no, Ak, bl, Ha, mJ, dx, Ad, Ff, gY, axV, oh, MW, bD, it, B, fG, aA, eA, ahL, aT, kf, rz, bsd, aTm, hM, aEk, aYp, pb, beH, beJ, apq, c, cHA, bt, VB, Pt, acl, wV, fu, aoz, hd, afm, oj, biX, Oq, xs, eZ, aF, Pd, dr, aeb, LB, axl, RQ, Hs, i); #c1(SMN2) c2(NP_075014) c3(survival motor neuron protein isoform b) c4(213516) c5(213517, 213518, 213519, 213520, 213521, 213522, 213523, 213524, 213525, 213526, 213527, 213528, 213529, 213530, 213531) c6( ) c7(bM, T, boQ, aey, agU, Vd, ci, arX, x, K, bU, ass, apl, Hl, aW, MV, cm, aoS, bdx, ly, eM, cHz, aem, U, cHy, k, Ro, Ol, n, fr, b, no, Ak, bl, Ha, mJ, dx, Ad, Ff, gY, axV, oh, MW, bD, it, B, fG, aA, eA, ahL, aT, kf, rz, bsd, aTm, hM, aEk, aYp, pb, beH, beJ, apq, c, cHA, bt, VB, Pt, acl, wV, fu, aoz, hd, afm, oj, biX, Oq, xs, eZ, aF, Pd, dr, aeb, LB, axl, RQ, Hs, i); #c1(SMN2) c2(XP_011541901) c3(survival motor neuron protein isoform X1) c4(340425) c5(340426, 340427, 340428, 340429, 340430, 340431, 340432, 340433, 340434, 340435, 340436) c6( ) c7(bM, T, boQ, aey, agU, Vd, ci, arX, x, K, bu, ass, apl, Hl, aW, MV, cm, aoS, bdx, ly, eM, cHz, aem, U, cHy, k, Ro, Ol, n, fr, b, no, Ak, bl, Ha, mJ, dx, Ad, Ff, gY, axV, oh, MW, bD, it, B, fG, aA, eA, ahL, aT, kf, rz, bsd, aTm, hM, aEk, aYp, pb, beH, beJ, apq, c, cHA, bt, VB, Pt, acl, wV, fu, aoz, hd, afm, oj, biX, Oq, xs, eZ, aF, Pd, dr, aeb, LB, axl, RQ, Hs, i); #c1(SMN2) c2(XP_011541902) c3(survival motor neuron protein isoform X2) c4(340437) c5(340438, 340439, 340440, 340441, 340442, 340443, 340444, 340445, 340446, 340447, 340448) c6( ) c7(bM, T, boQ, aey, agU, Vd, ci, arX, x, K, bU, ass, apl, Hl, aW, MV, cm, aoS, bdx, ly, eM, cHz, aem, U, cHy, k, Ro, Ol, n, fr, b, no, Ak, bl, Ha, mJ, dx, Ad, Ff, gY, axV, oh, MW, bD, it, B, fG, aA, eA, ahL, aT, kf, rz, bsd, aTm, hM, aEk, aYp, pb, beH, beJ, apq, c, cHA, bt, VB, Pt, acl, wV, fu, aoz, hd, afm, oj, biX, Oq, xs, eZ, aF, Pd, dr, aeb, LB, axl, RQ, Hs, i); #c1(SMO) c2(NP_005622) c3(smoothened homolog precursor) c4(158029) c5(158030, 158031, 158032, 158033, 158034, 158035) c6( ) c7(gh, T, kh, aC, pf, aD, aA, hy, dG, ci, auG, q, ad, ip, bv, av, ib, afQ, aT, dL, K, jj, pk, fb, ae, jf, BN, Qr, nO, aW, ff, eH, wF, CT, aF, en, g, eF, iV, bN, ar, sS, dB, hk, bE, aSp, YH, ne, hS); #c1(SMOC1) c2(NP_001030024) c3(SPARC-related modular calcium-binding protein 1 isoform 1 precursor) c4(30881) c5(30882, 30883, 30884, 30885, 30886, 30887) c6( ) c7(Na, aC, A, iU, tT, cHB, aiM, lo, kp, zU, cm, fq, pb, akE); #c1(SMOC1) c2(NP_071420) c3(SPARC-related modular calcium-binding protein 1 isoform 2 precursor) c4(211565) c5(211566, 211567, 211568, 211569, 211570, 211571) c6( ) c7(Na, aC, A, iU, tT, cHB, aiM, lo, kp, zu, cm, fq, pb, akE); #c1(SMOC2) c2(NP_001159884) c3(SPARC-related modular calcium-binding protein 2 isoform 2 precursor) c4(64874) c5(64875, 64876, 64877, 64878, 64879, 64880, 64881, 64882, 64883, 64884, 64885, 64886) c6( ) c7(bM, yl, aw, jx, hz, qj, pC); #c1(SMOC2) c2(NP_071421) c3(SPARC-related modular calcium-binding protein 2 isoform 1 precursor) c4(211572) c5(211573, 211574, 211575, 211576, 211577, 211578) c6( ) c7(bM, yl, aw, jx, hz, qj, pC); #c1(SMOC2) c2(XP_011534367) c3(SPARC-related modular calcium-binding protein 2 isoform X1) c4(330770) c5( ) c6( ) c7(bM, yl, aw, jx, hz, qj, pC); #c1(SMOC2) c2(XP_011534368) c3(SPARC-related modular calcium-binding protein 2 isoform X2) c4(330771) c5(330772, 330773, 330774, 330775, 330776, 330777) c6( ) c7(bM, yl, aw, jx, hz, qj, pC); #c1(SMOX) c2(NP_787033) c3(spermine oxidase isoform 1) c4(251957) c5(251958, 251959, 251960, 251961, 251962, 251963, 251964, 251965, 251966, 251967) c6( ) c7(gh, Kd, T, pf, aD, NT, aA, bS, ww, C, q, ad, ip, bv, av, ib, aT, h, K, pk, fb, ae, jf, Qr, nO, aW, Ue, ff, az, eH, aF, en, Sm, g, eF, iV, sS, ac, bE, fD, YH, ao, hS); #c1(SMOX) c2(NP_787034) c3(spermine oxidase isoform 2) c4(251968) c5(251969, 251970, 251971, 251972, 251973, 251974) c6( ) c7(gh, Kd, T, pf, aD, NT, aA, bS, ww, C, q, ad, ip, bv, av, ib, aT, h, K, pk, fb, ae, jf, Qr, nO, aW, Ue, ff, az, eH, af, en, Sm, g, eF, iV, sS, ac, bE, fD, YH, ao, hS); #c1(SMOX) c2(NP_787035) c3(spermine oxidase isoform 3) c4(251975) c5(251976, 251977, 251978, 251979, 251980, 251981) c6( ) c7(gh, Kd, T, pf, aD, NT, aA, bS, ww, C, q, ad, ip, bv, av, ib, aT, h, K, pk, fb, ae, jf, Qr, nO, aW, Ue, ff, az, eH, af, en, Sm, g, eF, iV, sS, ac, bE, fD, YH, ao, hS); #c1(SMOX) c2(NP_787036) c3(spermine oxidase isoform 4) c4(251982) c5(251983, 251984, 251985, 251986, 251987, 251988) c6( ) c7(gh, Kd, T, pf, aD, NT, aA, bS, ww, C, q, ad, ip, bv, av, ib, aT, h, K, pk, fb, ae, jf, Qr, nO, aW, Ue, ff, az, eH, aF, en, Sm, g, eF, iV, sS, ac, bE, fD, YH, ao, hS); #c1(SMOX) c2(XP_011527563) c3(spermine oxidase isoform X1) c4(322403) c5(322404, 322405, 322406, 322407, 322408, 322409) c6( ) c7(gh, Kd, T, pf, aD, NT, aA, bS, ww, C, q, ad, ip, bv, av, ib, aT, h, K, pk, fb, ae, jf, Qr, nO, aW, Ue, ff, az, eH, af, en, Sm, g, eF, iV, sS, ac, bE, fD, YH, ao, hS); #c1(SMPD1) c2(NP_000534) c3 (sphingomyelin phosphodiesterase isoform 1 precursor) c4(12058) c5(12059, 12060, 12061, 12062, 12063, 12064, 12065, 12066, 12067, 12068, 12069, 12070, 12071, 12072, 12073, 12074, 12075, 12076, 12077, 12078, 12079, 12080, 12081, 12082, 12083, 12084, 12085, 12086, 12087, 12088, 12089, 12090, 12091, 12092, 12093, 12094, 12095, 12096, 12097, 12098, 12099, 12100, 12101, 12102, 12103, 12104, 12105, 12106, 12107, 12108, 12109, 12110, 12111, 12112, 12113, 12114, 12115, 12116, 12117, 12118, 12119, 12120, 12121, 12122, 12123, 12124, 12125, 12126, 12127, 12128, 12129, 12130, 12131, 12132, 12133, 12134, 12135, 12136, 12137, 12138, 12139, 12140, 12141, 12142, 12143, 12144, 12145, 12146, 12147, 12148, 12149, 12150, 12151, 12152, 12153, 12154, 12155) c6(12156, 12157, 12158) c7(kV, oO, T, bM, aPU, bB, bS, j, ct, v, Rk, aR, aT, dL, K, cHC, bq, lQ, he, an, vl, ao, Y, clk, PX, hl, aq, Z, eE, iR, dr, vU, eY, bE, dh, ajn, vy, cG, at, akH, Lq, bt, sH, ek); #c1(SMPD1) c2(NP_001007594) c3(sphingomyelin phosphodiesterase isoform 2 precursor) c4(22835) c5(22836, 22837, 22838, 22839, 22840, 22841, 22842, 22843, 22844, 22845) c6(22846, 22847) c7(kV, oO, T, bM, aPU, bB, bS, j, ct, v, Rk, aR, aT, dL, K, cHC, bq, lQ, he, an, vl, ao, Y, clk, PX, hl, aq, Z, eE, iR, dr, vU, eY, bE, dh, ajn, vy, cG, at, akH, Lq, bt, sH, ek); #c1(SMPD1) c2(XP_011518605) c3(sphingomyelin phosphodiesterase isoform X1) c4(312779, 312780) c5(312781, 312782, 312783, 312784, 312785, 312786, 312787, 312788, 312789, 312790, 312791, 312792, 312793, 312794, 312795, 312796, 312797, 312798, 312799) c6(312800, 312801, 312802, 312803) c7(kV, oO, T, bM, aPU, bB, bS, j, ct, v, Rk, aR, aT, dL, K, cHC, bq, lQ, he, an, vl, ao, Y, clk, PX, hl, aq, Z, eE, iR, dr, vU, eY, bE, dh, ajn, vy, cG, at, akH, Lq, bt, sH, ek); #c1(SMPD1) c2(XP_011518606) c3(sphingomyelin phosphodiesterase isoform X3) c4(312804, 312805) c5(312806, 312807, 312808, 312809, 312810, 312811, 312812, 312813, 312814, 312815, 312816, 312817, 312818, 312819, 312820, 312821, 312822) c6(312823, 312824, 312825, 312826) c7(kV, oO, T, bM, aPU, bB, bS, j, ct, v, Rk, aR, aT, dL, K, cHC, bq, lQ, he, an, vl, ao, Y, clk, PX, hl, aq, Z, eE, iR, dr, vU, eY, bE, dh, ajn, vy, cG, at, akH, Lq, bt, sH, ek); #c1(SMPD2) c2(NP_003071) c3(sphingomyelin phosphodiesterase 2) c4(134976) c5(134977, 134978, 134979, 134980, 134981, 134982) c6( ) c7(ck, T, bE, aW, aT, bB, K, eE, eA, ek); #c1(SMPD3) c2(NP_061137) c3(sphingomyelin phosphodiesterase 3) c4(199484) c5(199485, 199486, 199487, 199488, 199489, 199490) c6( ) c7(ck, T, bBJ, avP, aW, aT, hn, bw, bt); #c1(SMPDL3A) c2(NP_001273067) c3(acid sphingomyelinase-like phosphodiesterase 3a isoform b) c4(104288) c5(104289, 104290, 104291, 104292, 104293) c6( ) c7(dP, dL); #c1(SMPDL3A) c2(NP_006705) c3(acid sphingomyelinase-like phosphodiesterase 3a isoform a precursor) c4(167298) c5(167299, 167300, 167301, 167302, 167303, 167304) c6( ) c7(dP, dL); #c1(SMPDL3A) c2(XP_005266854) c3(acid sphingomyelinase-like phosphodiesterase 3a isoform X1) c4( ) c5(280363) c6( ) c7(dP, dL); #c1(SMPDL3B) c2(XP_011539561) c3(acid sphingomyelinase-like phosphodiesterase 3b isoform X1) c4(337790) c5(337791, 337792, 337793, 337794, 337795, 337796, 337797, 337798) c6(337799, 337800) c7(sk); #c1(SMPX) c2(NP_055147) c3(small muscular protein) c4(179442) c5(179443, 179444, 179445, 179446, 179447, 179448) c6( ) c7(K, cHD); #c1(SMR3B) c2(NP_006676) c3(submaxillary gland androgen-regulated protein 3B precursor) c4(167070, 167071) c5(167072, 167073, 167074, 167075, 167076, 167077, 167078, 167079, 167080, 167081, 167082, 167083, 167084, 167085, 167086, 167087, 167088, 167089, 167090, 167091, 167092, 167093, 167094, 167095) c6( ) c7(T, aA, bB, n, fd, ci, q, hX, aT, cp, cY, iU, aW, Y, r, aF, g, cm, bz, ck, bE, bt, it, B); #c1(SMS) c2(NP_001245352) c3(spermine synthase isoform 2) c4(93221) c5(93222, 93223, 93224, 93225, 93226, 93227) c6( ) c7(OF, Kk, ha, ml, amj, bS, nA, rc, blg, bK, K, Yi, bA, jf, cHE, Y, LO, nf, gl, cm, xw, hq, cA, Km); #c1(SMS) c2(NP_004586) c3(spermine synthase isoform 1) c4(147914, 147915, 147916) c5(147917, 147918, 147919, 147920, 147921, 147922, 147923, 147924, 147925, 147926, 147927, 147928) c6( ) c7(OF, Kk, ha, ml, amj, bS, nA, rc, blg, bK, K, Yi, bA, jf, cHE, Y, LO, nf, gl, cm, xw, hq, cA, Km); #c1(SMS) c2(XP_011543870) c3(spermine synthase isoform X1) c4( ) c5(343027, 343028, 343029, 343030, 343031, 343032) c6( ) c7(OF, Kk, ha, ml, amj, bS, nA, rc, blg, bK, K, Yi, bA, jf, cHE, Y, LO, nf, gl, cm, xw, hq, cA, Km); #c1(SMTN) c2(NP_008863) c3(smoothelin isoform c) c4(169310) c5( ) c6( ) c7(DQ, aC, K); #c1(SMTN) c2(NP_599031) c3(smoothelin isoform b) c4(235622, 235623) c5( ) c6( ) c7(DQ, aC, K); #c1(SMTN) c2(NP_599032) c3(smoothelin isoform a) c4(235624) c5(235625, 235626, 235627, 235628, 235629, 235630) c6( ) c7(DQ, aC, K); #c1(SMTN) c2(XP_011528634) c3(smoothelin isoform X1) c4( ) c5(323885, 323886, 323887, 323888, 323889) c6( ) c7(DQ, aC, K); #c1(SMTN) c2(XP_011528635) c3(smoothelin isoform X2) c4(323890) c5(323891, 323892, 323893, 323894, 323895, 323896) c6( ) c7(DQ, aC, K); #c1(SMTN) c2(XP_011528636) c3(smoothelin isoform X3) c4( ) c5(323897, 323898, 323899, 323900, 323901, 323902) c6( ) c7(DQ, aC, K); #c1(SMTN) c2(XP_011528637) c3(smoothelin isoform X4) c4( ) c5(323903, 323904, 323905, 323906, 323907, 323908, 323909, 323910, 323911, 323912, 323913, 323914) c6( ) c7(DQ, aC, K); #c1(SMTN) c2(XP_011528639) c3(smoothelin isoform X7) c4(323915) c5(323916, 323917, 323918, 323919, 323920, 323921) c6( ) c7(DQ, aC, K); #c1(SMTN) c2(XP_011528641) c3(smoothelin isoform X10) c4(323922) c5( ) c6( ) c7(DQ, aC, K); #c1(SMTN) c2(XP_011528642) c3(smoothelin isoform X11) c4( ) c5(323923) c6( ) c7(DQ, aC, K); #c1(SMTNL1) c2(NP_001099035) c3(smoothelin-like protein 1) c4(41715, 41716, 41717) c5(41718, 41719, 41720, 41721, 41722, 41723, 41724, 41725, 41726, 41727, 41728, 41729, 41730, 41731, 41732, 41733, 41734, 41735) c6( ) c7(T); #c1(SMU1) c2(NP_060695) c3(WD40 repeat-containing protein SMU1) c4(197271, 197272) c5(197273, 197274, 197275, 197276, 197277, 197278) c6( ) c7(aC); #c1(SMU1) c2(XP_005251560) c3(WD40 repeat-containing protein SMU1 isoform X1) c4( ) c5(270999, 271000, 271001, 271002, 271003, 271004) c6( ) c7(aC); #c1(SMUG1) c2(XP_006719385) c3(single-strand selective monofunctional uracil DNA glycosylase isoform X2) c4(292439) c5(292440, 292441, 292442, 292443, 292444, 292445, 292446, 292447, 292448, 292449, 292450, 292451, 292452, 292453, 292454, 292455, 292456, 292457) c6( ) c7(bL, lD, T, dt, Mb, if, ha, NT, bO, aA, bB, n, it, ci, qN, yU, V, fK, q, eA, chj, iu, ku, b, hd, aE, i, sO, ajv, aT, yT, yZ, fb, af, uQ, ly, agT, aW, anx, eH, ff, r, aF, en, g, eF, t, bz, lx, agS, qJ, Qy, bb, nM, c, nB, bE, eM, OG, FJ, uo, m, qF, R, ij, zu, xg, B); #c1(SMUG1) c2(XP_011536423) c3(single-strand selective monofunctional uracil DNA glycosylase isoform X1) c4(333729, 333730) c5(333731, 333732, 333733, 333734, 333735, 333736, 333737, 333738, 333739, 333740, 333741, 333742, 333743, 333744, 333745, 333746, 333747, 333748, 333749, 333750) c6( ) c7(bL, lD, T, dt, Mb, if, ha, NT, bO, aA, bB, n, it, ci, qN, yU, V, fK, q, eA, chj, iu, ku, b, hd, aE, i, sO, ajv, aT, yT, yZ, fb, af, uQ, ly, agT, aW, anx, eH, ff, r, aF, en, g, eF, t, bz, lx, agS, qJ, Qy, bb, nM, c, nB, bE, eM, OG, FJ, uo, m, qF, R, ij, zu, xg, B); #c1(SMUG1) c2(XP_011536425) c3(single-strand selective monofunctional uracil DNA glycosylase isoform X3) c4(333751, 333752, 333753, 333754) c5(333755, 333756, 333757, 333758, 333759, 333760, 333761, 333762, 333763, 333764, 333765, 333766, 333767, 333768, 333769, 333770, 333771, 333772, 333773, 333774, 333775, 333776, 333777, 333778) c6( ) c7(bL, lD, T, dt, Mb, if, ha, NT, bO, aA, bB, n, it, ci, qN, yU, V, fK, q, eA, chj, iu, ku, b, hd, aE, i, sO, ajv, aT, yT, yZ, fb, af, uQ, ly, agT, aW, anx, eH, ff, r, aF, en, g, eF, t, bz, lx, agS, qJ, Qy, bb, nM, c, nB, bE, eM, OG, FJ, uo, m, qF, R, ij, zu, xg, B); #c1(SMURF1) c2(NP_065162) c3(E3 ubiquitin-protein ligase SMURF1 isoform 1) c4(204355) c5(204356, 204357, 204358, 204359, 204360, 204361) c6( ) c7(T, aC, aE, aW, r, aT, aq, By, oH, lR, iV, iR, pb); #c1(SMURF1) c2(NP_851994) c3(E3 ubiquitin-protein ligase SMURF1 isoform 2) c4(254728) c5(254729, 254730, 254731, 254732, 254733, 254734) c6( ) c7(T, aC, aE, aW, r, aT, aq, By, oH, lR, iV, iR, pb); #c1(SMURF2) c2(NP_073576) c3(E3 ubiquitin-protein ligase SMURF2) c4(212924) c5(212925, 212926, 212927, 212928, 212929, 212930) c6( ) c7(T, kP, bP, aW, ff, U, aT, dy, Hb, en, lR, fr, AO, pb); #c1(SMYD1) c2(NP_938015) c3(histone-lysine N-methyltransferase SMYD1) c4(259512) c5(259513, 259514, 259515, 259516, 259517, 259518) c6( ) c7(bz, iu); #c1(SMYD1) c2(XP_005264213) c3(histone-lysine N-methyltransferase SMYD1 isoform X1) c4(278705) c5(278706, 278707, 278708, 278709, 278710, 278711) c6( ) c7(bz, iu); #c1(SMYD2) c2(NP_064582) c3(N-lysine methyltransferase SMYD2) c4(203098, 203099) c5(203100, 203101, 203102, 203103, 203104, 203105, 203106, 203107, 203108, 203109, 203110, 203111) c6( ) c7(x, ci, T, k); #c1(SMYD3) c2(NP_001161212) c3(histone-lysine N-methyltransferase SMYD3 isoform 1) c4(65504, 65505) c5(65506, 65507, 65508, 65509, 65510, 65511, 65512, 65513) c6( ) c7(T, eG, nv, aW, ff, ht, xa, aT, r, cd, aq, it, bK, ci, q, en, ae, B, ad); #c1(SMYD3) c2(NP_073580) c3(histone-lysine N-methyltransferase SMYD3 isoform 2) c4(212954) c5(212955, 212956, 212957, 212958, 212959, 212960, 212961, 212962, 212963, 212964, 212965, 212966, 212967, 212968, 212969, 212970, 212971, 212972) c6( ) c7(T, eG, nv, aW, ff, ht, xa, aT, r, cd, aq, it, bK, ci, q, en, ae, B, ad); #c1(SMYD3) c2(XP_011542555) c3 (histone-lysine N-methyltransferase SMYD3 isoform X1) c4(341107, 341108) c5(341109, 341110) c6( ) c7(T, eG, nv, aW, ff, ht, xa, aT, r, cd, aq, it, bK, ci, q, en, ae, B, ad); #c1(SMYD3) c2(XP_011542557) c3(histone-lysine N-methyltransferase SMYD3 isoform X3) c4( ) c5(341111) c6( ) c7(T, eG, nv, aW, ff, ht, xa, aT, r, cd, aq, it, bK, ci, q, en, ae, B, ad); #c1(SMYD3) c2(XP_011542559) c3(histone-lysine N-methyltransferase SMYD3 isoform X5) c4(341112) c5(341113, 341114, 341115, 341116, 341117, 341118) c6( ) c7(T, eG, nv, aW, ff, ht, xa, aT, r, cd, aq, it, bK, ci, q, en, ae, B, ad); #c1(SMYD3) c2(XP_011542563) c3(histone-lysine N-methyltransferase SMYD3 isoform X6) c4( ) c5(341119, 341120, 341121, 341122, 341123) c6( ) c7(T, eG, nv, aW, ff, ht, xa, aT, r, cd, aq, it, bK, ci, q, en, ae, B, ad); #c1(SMYD4) c2(NP_443160) c3(SET and MYND domain-containing protein 4) c4(229677) c5(229678, 229679, 229680, 229681, 229682, 229683) c6( ) c7(jf, aW, j, aT); #c1(SMYD4) c2(XP_011521935) c3(SET and MYND domain-containing protein 4 isoform X2) c4( ) c5(316637) c6( ) c7(jf, aW, j, aT); #c1(SMYD5) c2(NP_006053) c3(SET and MYND domain-containing protein 5) c4(161505) c5(161506, 161507, 161508, 161509, 161510, 161511, 161512) c6( ) c7(ck, eM, aW, aT, gE, jj, B); #c1(SNAI1) c2(NP_005976) c3(zinc finger protein SNAI1) c4(160931) c5(160932, 160933, 160934, 160935, 160936, 160937, 160938) c6( ) c7(T, aA, cz, n, hy, v, bB, ci, fr, Lw, PJ, Qo, agY, AO, q, ad, eA, hX, iJ, hd, aE, Db, yt, aT, io, rO, rD, K, hB, ae, jf, d, BN, B, aW, Y, ff, eH, r, aF, en, cl, hJ, t, eF, bz, zV, LB, eM, iA, dB, bE, X, at, aPG, Hb, m, ei, ne); #c1(SNAI2) c2(NP_003059) c3(zinc finger protein SNAI2) c4(134806) c5(134807, 134808, 134809, 134810, 134811, 134812, 134813, 134814, 134815, 134816, 134817, 134818, 134819) c6(134820, 134821) c7(cHF, T, ht, it, bB, n, amj, v, fr, ci, bjN, Qo, q, ad, eA, asG, hd, ib, jz, hW, yt, Db, aT, aAE, ahE, ae, d, aW, Y, ff, eH, ahl, en, cl, g, t, eF, bz, cm, LB, bE, np, m, aPG, ik, B); #c1(SNAI3) c2(NP_840101) c3(zinc finger protein SNAI3) c4(253628) c5(253629, 253630, 253631, 253632, 253633, 253634, 253635) c6( ) c7(Y, wz); #c1(SNAP23) c2(NP_003816) c3(synaptosomal-associated protein 23 isoform SNAP23A) c4(141104) c5(141105, 141106, 141107, 141108, 141109, 141110) c6( ) c7(bL, YE, s, j, pz, iJ); #c1(SNAP23) c2(NP_570710) c3(synaptosomal-associated protein 23 isoform SNAP23B) c4(234135) c5(234136, 234137, 234138, 234139, 234140, 234141, 234142, 234143, 234144, 234145) c6( ) c7(bL, YE, s, j, pz, iJ); #c1(SNAP25) c2(NP_003072) c3(synaptosomal-associated protein 25 isoform SNAP25A) c4(134983) c5(134984, 134985, 134986, 134987, 134988, 134989) c6( ) c7(bL, T, id, iH, lT, Or, e, ha, bO, jh, Jd, cHG, ct, nA, afE, cd, aR, x, cF, no, KB, aE, WY, kH, h, dL, bK, ox, wb, bq, H, he, rz, af, eL, na, bA, iz, je, s, akL, Sj, f, cm, ck, kn, pH, bb, YE, c, iM, acw, k, bC, ei); #c1(SNAP25) c2(NP_570824) c3(synaptosomal-associated protein 25 isoform SNAP25B) c4(234281, 234282) c5(234283, 234284, 234285, 234286, 234287, 234288, 234289, 234290, 234291, 234292, 234293, 234294) c6( ) c7(bL, T, id, iH, lT, Or, e, ha, bO, jh, Jd, cHG, ct, nA, afE, cd, aR, x, cF, no, KB, aE, WY, kH, h, dL, bK, ox, wb, bq, H, he, rz, af, eL, na, bA, iz, je, s, akL, Sj, f, cm, ck, kn, pH, bb, YE, c, iM, acw, k, bC, ei); #c1(SNAP29) c2(NP_004773) c3(synaptosomal-associated protein 29) c4(149504) c5(149505, 149506, 149507, 149508, 149509, 149510) c6( ) c7(v, fp, cHH, lQ, by); #c1(SNAP47) c2(NP_444280) c3(synaptosomal-associated protein 47) c4(230260, 230261, 230262) c5(230263, 230264, 230265, 230266, 230267, 230268, 230269, 230270, 230271, 230272, 230273, 230274, 230275, 230276, 230277, 230278, 230279, 230280) c6( ) c7(vx, K); #c1(SNAP47) c2(XP_005273101) c3(synaptosomal-associated protein 47 isoform X3) c4(284292) c5(284293, 284294, 284295, 284296, 284297) c6( ) c7(vx, K); #c1(SNAP47) c2(XP_006711797) c3(synaptosomal-associated protein 47 isoform X2) c4(286232) c5(286233, 286234, 286235, 286236, 286237, 286238, 286239) c6( ) c7(vx, K); #c1(SNAP91) c2(NP_001229721) c3(clathrin coat assembly protein AP180 isoform a) c4(84902, 84903) c5(84904, 84905, 84906, 84907, 84908, 84909, 84910, 84911, 84912, 84913, 84914, 84915, 84916, 84917, 84918, 84919, 84920, 84921, 84922, 84923) c6( ) c7(ck, Na, T, iT, amJ, rO, Nc, gE, hf, hF, bt); #c1(SNAP91) c2(NP_001229722) c3(clathrin coat assembly protein AP180 isoform b) c4(84924) c5(84925, 84926, 84927, 84928, 84929, 84930) c6( ) c7(ck, Na, T, iT, amJ, rO, Nc, gE, hf, hF, bt); #c1(SNAP91) c2(NP_001229723) c3(clathrin coat assembly protein AP180 isoform c) c4(84931, 84932) c5(84933, 84934, 84935, 84936, 84937, 84938, 84939, 84940, 84941, 84942, 84943, 84944, 84945, 84946, 84947, 84948, 84949, 84950, 84951, 84952, 84953, 84954, 84955, 84956, 84957, 84958, 84959, 84960, 84961, 84962, 84963, 84964, 84965, 84966, 84967, 84968, 84969, 84970, 84971, 84972, 84973, 84974, 84975, 84976, 84977, 84978) c6( ) c7(ck, Na, T, iT, amJ, rO, Nc, gE, hf, hF, bt); #c1(SNAP91) c2(NP_001243646) c3(clathrin coat assembly protein AP180 isoform d) c4( ) c5(91307) c6( ) c7(ck, Na, T, iT, amJ, r, Nc, gE, hf, hF, bt); #c1(SNAP91) c2(XP_011534571) c3(clathrin coat assembly protein AP180 isoform X4) c4(331106, 331107) c5(331108, 331109, 331110, 331111, 331112, 331113, 331114, 331115, 331116, 331117) c6( ) c7(ck, Na, T, iT, amJ, rO, Nc, gE, hf, hF, bt); #c1(SNAP91) c2(XP_011534573) c3(clathrin coat assembly protein AP180 isoform X6) c4(331118) c5( ) c6( ) c7(ck, Na, T, iT, amJ, rO, Nc, gE, hf, hF, bt); #c1(SNAP91) c2(XP_011534574) c3(clathrin coat assembly protein AP180 isoform X7) c4(331119) c5( ) c6( ) c7(ck, Na, T, iT, amJ, rO, Nc, gE, hf, hF, bt); #c1(SNAP91) c2(XP_011534578) c3(clathrin coat assembly protein AP180 isoform X11) c4(331120) c5(331121, 331122, 331123, 331124, 331125, 331126) c6( ) c7(ck, Na, T, iT, amJ, rO, Nc, gE, hf, hF, bt); #c1(SNAP91) c2(XP_011534582) c3(clathrin coat assembly protein AP180 isoform X15) c4(331127, 331128) c5( ) c6( ) c7(ck, Na, T, iT, amJ, rO, Nc, gE, hf, hF, bt); #c1(SNAPC1) c2(NP_003073) c3(snRNA-activating protein complex subunit 1) c4(134990) c5(134991, 134992, 134993, 134994, 134995, 134996, 134997) c6( ) c7(aT); #c1(SNAPC4) c2(NP_003077) c3(snRNA-activating protein complex subunit 4) c4(135017) c5(135018, 135019, 135020, 135021, 135022, 135023, 135024) c6( ) c7(mR); #c1(SNAPC5) c2(NP_006040) c3(snRNA-activating protein complex subunit 5) c4(161400, 161401) c5(161402, 161403, 161404, 161405, 161406, 161407, 161408, 161409, 161410, 161411, 161412, 161413, 161414, 161415, 161416, 161417) c6( ) c7(W); #c1(SNCA) c2(NP_009292) c3(alpha-synuclein isoform NACP112) c4(171772) c5(171773, 171774, 171775, 171776, 171777, 171778, 171779, 171780, 171781, 171782, 171783, 171784, 171785, 171786, 171787, 171788, 171789, 171790, 171791, 171792, 171793, 171794) c6( ) c7(T, aaH, yY, jx, GC, Gd, cHL, ct, alS, bfD, aR, ad, Hx, Gt, brS, cHK, HC, KH, aT, h, K, an, H, ae, xa, dF, alL, af, NN, ak, hZ, Jj, A, qZ, s, je, jf, cHJ, Y, aW, Ls, eZ, gl, cu, dg, lo, as, Rl, cm, xw, cHI, GB, FT, vU, bb, iM, c, Gs, U, uo, sv, aqm, CD, Lq, R, y, aen, CW, hv, L); #c1(SNCA) c2(XP_011530509) c3 (alpha-synuclein isoform X2) c4(326405) c5( ) c6( ) c7(T, aaH, yY, jx, GC, Gd, cHL, ct, alS, bfD, aR, ad, Hx, Gt, brS, cHK, HC, kH, aT, h, K, an, H, ae, xa, dF, alL, af, NN, ak, hZ, Jj, A, qZ, s, je, jf, cHJ, Y, aW, Ls, eZ, gl, cu, dg, lo, as, Rl, cm, xw, cHI, GB, FT, vU, bb, iM, c, Gs, U, uo, sv, aqm, CD, Lq, R, y, aen, CW, hv, L); #c1(SNCA) c2(XP_011530510) c3(alpha-synuclein isoform X1) c4(326406, 326407, 326408) c5(326409, 326410, 326411, 326412, 326413, 326414, 326415, 326416, 326417, 326418, 326419, 326420, 326421, 326422, 326423, 326424, 326425, 326426, 326427, 326428, 326429, 326430, 326431, 326432, 326433, 326434, 326435, 326436, 326437, 326438, 326439, 326440) c6( ) c7(T, aaH, yY, jx, GC, Gd, cHL, ct, alS, bfD, aR, ad, Hx, Gt, brS, cHK, HC, KH, aT, h, K, an, H, ae, xa, dF, alL, af, NN, ak, hZ, Jj, A, qZ, s, je, jf, cHJ, Y, aW, Ls, eZ, gl, cu, dg, lo, as, Rl, cm, xw, cHI, GB, FT, vU, bb, iM, c, Gs, U, uo, sv, aqm, CD, Lq, R, y, aen, CW, hv, L); #c1(SNCA1P) c2(NP_001229864) c3(synphilin-1 isoform 1A) c4( ) c5(85485, 85486, 85487, 85488, 85489) c6( ) c7(ak, U, GC, v, K, act, an, cm, GB); #c1(SNCA1P) c2(NP_005451) c3(synphilin-1 isoform 1) c4(156291, 156292) c5(156293, 156294, 156295, 156296, 156297, 156298) c6( ) c7(ak, U, GC, v, K, act, an, cm, GB); #c1(SNCA1P) c2(XP_011542047) c3(synphilin-1 isoform X3) c4(340571) c5( ) c6( ) c7(ak, U, GC, v, K, act, an, cm, GB); #c1(SNCA1P) c2(XP_011542048) c3(synphilin-1 isoform X4) c4(340572) c5( ) c6( ) c7(ak, U, GC, v, K, act, an, cm, GB); #c1(SNCA1P) c2(XP_011542051) c3(synphilin-1 isoform X7) c4(340573, 340574) c5(340575, 340576, 340577, 340578, 340579, 340580, 340581) c6( ) c7(ak, U, GC, v, K, act, an, cm, GB); #c1(SNCB) c2(NP_003076) c3(beta-synuclein) c4(134998) c5(134999, 135000, 135001, 135002, 135003, 135004, 135005, 135006, 135007, 135008, 135009, 135010, 135011, 135012, 135013, 135014, 135015, 135016) c6( ) c7(jf, ak, aDA, U, cm, K, an, AO, Fc, qW); #c1(SNCG) c2(NP_003078) c3(gamma-synuclein) c4(135025) c5(135026, 135027, 135028, 135029, 135030, 135031) c6( ) c7(T, al, bB, v, ct, ci, PJ, zP, q, hX, dP, av, Bl, iC, aT, dL, K, an, af, ak, zQ, aW, ff, r, aq, cu, en, dw, t, Px, iR, bP, bE, U, m, CW); #c1(SNCG) c2(XP_011538407) c3(gamma-synuclein isoform X1) c4(336086) c5(336087, 336088, 336089, 336090, 336091, 336092) c6( ) c7(T, al, bB, v, ct, ci, PJ, zP, q, hX, dP, av, Bl, iC, aT, dL, K, an, af, ak, zQ, aW, ff, r, aq, cu, en, dw, t, Px, iR, bP, bE, U, m, CW); #c1(SND1) c2(NP_055205) c3(staphylococcal nuclease domain-containing protein 1) c4(179804, 179805) c5(179806, 179807, 179808, 179809, 179810, 179811, 179812, 179813, 179814, 179815, 179816, 179817, 179818) c6( ) c7(bN, T, iM, je, bE, aW, go, jh, aT, ahC, KH, bB, fd, K, qN, ae, X, ad, pl); #c1(SNED1) c2(NP_001073906) c3(sushi, nidogen and EGF-like domain-containing protein 1 precursor) c4(36433, 36434, 36435, 36436) c5(36437, 36438, 36439, 36440, 36441, 36442, 36443, 36444, 36445, 36446, 36447, 36448, 36449, 36450, 36451, 36452, 36453, 36454, 36455, 36456, 36457, 36458, 36459, 36460, 36461, 36462, 36463, 36464, 36465, 36466, 36467, 36468) c6(36469, 36470, 36471, 36472, 36473, 36474, 36475, 36476) c7(jf, d, bE, wu, bB, aj, v, ci, wm, eA, J); #c1(SNED1) c2(XP_011509239) c3(sushi, nidogen and EGF-like domain-containing protein 1 isoform X6) c4( ) c5(299891) c6( ) c7(jf, d, bE, wu, bB, aj, v, ci, wm, eA, J); #c1 (SNF8) c2(NP_009172) c3(vacuolar-sorting protein SNF8) c4(171249) c5(171250, 171251, 171252, 171253, 171254, 171255) c6( ) c7(ck, T, dD, ea, h, eq, cm, bt); #c1(SNIP1) c2(XP_011540462) c3(smad nuclear-interacting protein 1 isoform X1) c4( ) c5(338932) c6( ) c7(eM, cHM, T, bz); #c1(SNRK) c2(NP_001094064) c3(SNF-related serine/threonine-protein kinase) c4(40588) c5(40589, 40590, 40591, 40592, 40593, 40594, 40595, 40596, 40597, 40598, 40599, 40600, 40601, 40602) c6( ) c7(bB, bE); #c1(SNRNP200) c2(NP_054733) c3(U5 small nuclear ribonucleoprotein 200 kDa helicase) c4(177423, 177424, 177425, 177426, 177427, 177428) c5(177429, 177430, 177431, 177432, 177433, 177434, 177435, 177436, 177437, 177438, 177439, 177440, 177441, 177442, 177443, 177444, 177445, 177446, 177447, 177448, 177449, 177450, 177451, 177452, 177453, 177454, 177455, 177456, 177457, 177458, 177459, 177460, 177461, 177462, 177463, 177464) c6( ) c7(nC, cHN, xX, lR, U); #c1(SNRNP27) c2(NP_006848) c3(U4/U6.U5 small nuclear ribonucleoprotein 27 kDa protein) c4(168598) c5(168599, 168600, 168601, 168602, 168603, 168604, 168605, 168606, 168607, 168608) c6( ) c7(IW); #c1(SNRNP70) c2(NP_001287998) c3(U1 small nuclear ribonucleoprotein 70 kDa isoform 2) c4(113350) c5(113351, 113352, 113353, 113354, 113355, 113356, 113357) c6( ) c7(bL, bM, aj, v, Kh, aGX, ww, eA, mC, J, ad, wG, bW, vH, dL, bk, rz, ae, eL, tb, eZ, abn, pb, uo, fL, ei); #c1(SNRNP70) c2(NP_003080) c3(U1 small nuclear ribonucleoprotein 70 kDa isoform 1) c4(135053, 135054) c5(135055, 135056, 135057, 135058, 135059, 135060, 135061, 135062, 135063, 135064) c6( ) c7(bL, bM, aj, v, Kh, aGX, ww, eA, mC, J, ad, wG, bW, vH, dL, bK, rz, ae, eL, tb, eZ, abn, pb, uo, fL, ei); #c1(SNRNP70) c2(XP_011525542) c3(U1 small nuclear ribonucleoprotein 70 kDa isoform X1) c4( ) c5(319969, 319970, 319971, 319972, 319973, 319974) c6( ) c7(bL, bM, aj, v, Kh, aGX, ww, eA, mC, J, ad, wG, bW, vH, dL, bK, rz, ae, eL, tb, eZ, abn, pb, uo, fL, ei); #c1(SNRPA) c2(NP_004587) c3(U1 small nuclear ribonucleoprotein A) c4(147929, 147930) c5(147931, 147932, 147933, 147934, 147935, 147936, 147937, 147938, 147939, 147940, 147941, 147942, 147943) c6( ) c7(aGX, mC, fL, uE, eZ); #c1(SNRPB) c2(NP_003082) c3(small nuclear ribonucleoprotein-associated proteins B and B′ isoform B) c4(135065, 135066) c5(135067, 135068, 135069, 135070, 135071, 135072, 135073, 135074, 135075, 135076, 135077, 135078, 135079, 135080, 135081, 135082, 135083, 135084, 135085, 135086, 135087, 135088, 135089, 135090) c6( ) c7(nC, aA, eZ, U, aT); #c1(SNRPB) c2(NP_937859) c3(small nuclear ribonucleoprotein-associated proteins B and B′ isoform B′) c4(259334) c5( ) c6( ) c7(nC, aA, eZ, U, aT); #c1(SNRPC) c2(NP_003084) c3(U1 small nuclear ribonucleoprotein C) c4(135091, 135092, 135093) c5(135094, 135095, 135096, 135097, 135098, 135099, 135100, 135101, 135102, 135103, 135104, 135105, 135106, 135107, 135108, 135109, 135110, 135111, 135112, 135113) c6( ) c7(eZ); #c1(SNRPD1) c2(NP_008869) c3(small nuclear ribonucleoprotein Sm D1 isoform 1) c4(169339) c5(169340, 169341, 169342, 169343, 169344, 169345) c6( ) c7(aY, eZ, fP); #c1(SNRPD3) c2(NP_004166) c3(small nuclear ribonucleoprotein Sm D3) c4(144000, 144001) c5(144002, 144003, 144004, 144005, 144006, 144007, 144008, 144009, 144010, 144011, 144012, 144013) c6( ) c7(h, zP, zQ, ci); #c1(SNRPE) c2(NP_001291393) c3(small nuclear ribonucleoprotein E isoform 2) c4( ) c5(115870, 115871, 115872, 115873, 115874, 115875) c6( ) c7(DS, T, AS, cHO, bB, bS, ci, Qp, q, ad, eA, zn, XX, av, aqt, hd, jz, aE, avA, aT, hP, K, fb, ae, eL, cp, atX, eG, yo, d, s, aW, Fm, Y, r, aq, yq, g, bz, Qt, iP, ck, es, nM, Dy, eM, nB, bE, Qu, CD, np, auN, ei, y, bt, it, X, On); #c1(SNRPE) c2(NP_003085) c3(small nuclear ribonucleoprotein E isoform 1) c4(135114, 135115, 135116) c5(135117, 135118, 135119, 135120, 135121, 135122, 135123, 135124, 135125, 135126, 135127, 135128) c6( ) c7(DS, T, AS, cHO, bB, bS, ci, Qp, q, ad, eA, zn, XX, av, aqt, hd, jz, aE, avA, aT, hP, K, fb, ae, eL, cp, atX, eG, yo, d, s, aW, Fm, Y, r, aq, yq, g, bz, Qt, iP, ck, es, nM, Dy, eM, nB, bE, Qu, CD, np, auN, ei, y, bt, it, X, On); #c1(SNRPF) c2(NP_003086) c3(small nuclear ribonucleoprotein F) c4(135129) c5(135130, 135131, 135132, 135133, 135134, 135135) c6( ) c7(bv, an); #c1(SNRPN) c2(NP_073719) c3(small nuclear ribonucleoprotein-associated protein N) c4(213219, 213220, 213221, 213222) c5(213223, 213224, 213225, 213226, 213227, 213228, 213229, 213230, 213231, 213232, 213233, 213234, 213235, 213236, 213237, 213238, 213239, 213240, 213241, 213242, 213243, 213244, 213245, 213246, 213247) c6( ) c7(T, gU, wu, Ol, ml, DA, KY, Vd, fu, hF, arX, q, rc, x, no, cHP, ahL, oj, Ak, kf, biX, rO, bl, K, wm, bU, he, rz, dx, ND, vT, apl, eZ, r, cm, dr, aeb, bN, axl, bdx, apq, c, aem, agl, U, axV, oh, cHy, MW, k, auK, bt); #c1(SNTA1) c2(NP_003089) c3(alpha-1-syntrophin) c4(135136) c5(135137, 135138, 135139, 135140, 135141, 135142) c6( ) c7(T, aW, aT, vc, EZ, Bu, cB, cHQ, mu); #c1(SNTB1) c2(NP_066301) c3(beta-1-syntrophin) c4(207141, 207142) c5(207143, 207144, 207145, 207146, 207147, 207148, 207149, 207150, 207151, 207152) c6( ) c7(b, e, Be, cz, akY, aj, i); #c1(SNTG1) c2(NP_001274743) c3 (gamma-1-syntrophin isoform 2) c4(106739) c5(106740, 106741, 106742, 106743, 106744, 106745, 106746, 106747, 106748, 106749) c6( ) c7(bL, W, hb, uY, rr, bQ, iu); #c1(SNTG1) c2(XP_011515849) c3(gamma-1-syntrophin isoform X1) c4(308816, 308817) c5(308818, 308819, 308820, 308821, 308822, 308823, 308824, 308825, 308826, 308827) c6( ) c7(bL, W, hb, uY, rr, bQ, iu); #c1(SNTG1) c2(XP_011515852) c3(gamma-1-syntrophin isoform X4) c4( ) c5(308828, 308829, 308830, 308831, 308832, 308833) c6( ) c7(bL, W, hb, uY, rr, bQ, iu); #c1(SNTG2) c2(NP_061841) c3(gamma-2-syntrophin) c4(200738) c5(200739, 200740, 200741, 200742, 200743, 200744) c6( ) c7(bS, VX, bN); #c1(SNUPN) c2(NP_005692) c3(snurportin-1) c4(158586, 158587) c5(158588, 158589, 158590, 158591, 158592, 158593, 158594, 158595, 158596, 158597, 158598, 158599, 158600, 158601, 158602, 158603) c6( ) c7(wR); #c1(SNURF) c2(NP_005669) c3(SNRPN upstream reading frame protein) c4(158399, 158400) c5(158401, 158402, 158403, 158404, 158405, 158406, 158407, 158408, 158409, 158410, 158411, 158412, 158413, 158414, 158415, 158416, 158417, 158418, 158419, 158420) c6( ) c7(rc); #c1(SNW1) c2(NP_036377) c3(SNW domain-containing protein 1) c4(173371) c5(173372, 173373, 173374, 173375, 173376, 173377, 173378) c6( ) c7(bM, T, Dy, aE, Y, Ex); #c1(SNW1) c2(XP_005267470) c3(SNW domain-containing protein 1 isoform X2) c4( ) c5(280990, 280991, 280992, 280993, 280994, 280995, 280996, 280997, 280998, 280999, 281000, 281001) c6( ) c7(bM, T, Dy, aE, Y, Ex); #c1(SNW1) c2(XP_005267471) c3(SNW domain-containing protein 1 isoform X1) c4(281002, 281003) c5( ) c6( ) c7(bM, T, Dy, aE, Y, Ex); #c1(SNX1) c2(NP_003090) c3(sorting nexin-1 isoform a) c4(135143, 135144) c5(135145, 135146, 135147, 135148, 135149, 135150, 135151, 135152, 135153, 135154, 135155, 135156) c6( ) c7(eM, bE, cz, r, bB, q, ci, bz, X, qa); #c1(SNX1) c2(NP_683758) c3(sorting nexin-1 isoform b) c4(242536) c5(242537, 242538, 242539, 242540, 242541, 242542, 242543, 242544, 242545) c6( ) c7(eM, bE, cz, r, bB, q, ci, bz, X, qa); #c1(SNX10) c2(NP_001186764) c3(sorting nexin-10 isoform 1) c4(78536, 78537) c5(78538, 78539, 78540, 78541, 78542, 78543, 78544, 78545, 78546, 78547) c6( ) c7(zw, ac, pN, zs, ey, an, cHR, bA); #c1(SNX10) c2(NP_001186766) c3(sorting nexin-10 isoform 2) c4( ) c5(78548) c6( ) c7(zw, ac, pN, zs, ey, an, cHR, bA); #c1(SNX10) c2(NP_001186767) c3(sorting nexin-10 isoform 3) c4( ) c5(78549, 78550, 78551, 78552, 78553) c6( ) c7(zw, ac, pN, zs, ey, an, cHR, bA); #c1(SNX12) c2(NP_001243117) c3(sorting nexin-12 isoform 4) c4(89969) c5(89970, 89971, 89972, 89973, 89974, 89975) c6( ) c7(af); #c1(SNX12) c2(NP_037478) c3(sorting nexin-12 isoform 1) c4(175731) c5(175732, 175733, 175734, 175735, 175736, 175737) c6( ) c7(af); #c1(SNX14) c2(NP_001284543) c3(sorting nexin-14 isoform c) c4(112028) c5(112029, 112030, 112031, 112032, (12033, 112034) c6( ) c7(Wl, nA); #c1(SNX14) c2(NP_001291408) c3(sorting nexin-14 isoform d) c4(115935) c5( ) c6( ) c7(Wl, nA); #c1(SNX14) c2(NP_065201) c3(sorting nexin-14 isoform b) c4(204561) c5(204562, 204563, 204564, 204565, 204566, 204567) c6( ) c7(Wl, nA); #c1(SNX14) c2(NP_722523) c3(sorting nexin-14 isoform a) c4(246800) c5(246801, 246802, 246803, 246804, 246805, 246806, 246807, 246808, 246809, 246810, 246811, 246812, 246813) c6( ) c7(Wl, nA); #c1(SNX14) c2(XP_011534280) c3(sorting nexin-14 isoform X4) c4(330647) c5( ) c6( ) c7(Wl, nA); #c1(SNX14) c2(XP_011534282) c3(sorting nexin-14 isoform X6) c4( ) c5(330648, 330649, 330650, 330651, 330652) c6( ) c7(Wl, nA); #c1(SNX16) c2(NP_690049) c3(sorting nexin-16 isoform a) c4(244828, 244829, 244830, 244831) c5(244832, 244833, 244834, 244835, 244836, 244837, 244838, 244839, 244840, 244841) c6( ) c7(ad, pZ); #c1(SNX16) c2(NP_690050) c3(sorting nexin-16 isoform b) c4(244842) c5(244843, 244844, 244845, 244846, 244847, 244848) c6( ) c7(ad, pZ); #c1(SNX16) c2(XP_005251341) c3(sorting nexin-16 isoform X3) c4(270885) c5(270886, 270887, 270888, 270889, 270890, 270891, 270892, 270893, 270894, 270895, 270896, 270897, 270898, 270899, 270900, 270901, 270902, 270903, 270904, 270905, 270906, 270907, 270908, 270909) c6( ) c7(ad, pZ); #c1(SNX18) c2(NP_001096045) c3(sorting nexin-18 isoform a) c4(40989) c5(40990, 40991, 40992, 40993, 40994, 40995, 40996) c6( ) c7(hH); #c1(SNX18) c2(NP_001138899) c3(sorting nexin-18 isoform c) c4( ) c5(58115) c6( ) c7(hH); #c1(SNX18) c2(NP_443102) c3(sorting nexin-18 isoform b) c4(229433, 229434) c5(229435, 229436, 229437, 229438, 229439, 229440, 229441, 229442, 229443, 229444, 229445) c6( ) c7(hH); #c1(SNX19) c2(NP_001288018) c3(sorting nexin-19 isoform 2) c4(113432) c5(113433, 113434, 113435, 113436, 113437) c6( ) c7(dD, ac, ea, e, aw, eq, j, eu); #c1(SNX19) c2(NP_055573) c3(sorting nexin-19 isoform 1) c4(182023, 182024) c5(182025, 182026, 182027, 182028, 182029, 182030, 182031, 182032, 182033, 182034, 182035, 182036, 182037) c6( ) c7(dD, ac, ea, e, aw, eq, j, eu); #c1(SNX2) c2(NP_003091) c3(sorting nexin-2 isoform 1) c4(135157) c5(135158, 135159, 135160, 135161, 135162, 135163) c6( ) c7(bz, ey); #c1(SNX20) c2(NP_001138444) c3(sorting nexin-20 isoform 3) c4(56799) c5(56800, 56801, 56802, 56803, 56804, 56805) c6( ) c7(fe, AW); #c1(SNX20) c2(NP_699168) c3(sorting nexin-20 isoform 2) c4(245799) c5(245800, 245801, 245802, 245803, 245804, 245805) c6( ) c7(fe, AW); #c1(SNX20) c2(NP_878274) c3(sorting nexin-20 isoform 1) c4(257078, 257079) c5(257080, 257081, 257082, 257083, 257084, 257085, 257086, 257087, 257088, 257089, 257090, 257091) c6( ) c7(fe, AW); #c1(SNX24) c2(NP_054754) c3(sorting nexin-24) c4( ) c5(177626, 177627, 177628, 177629, 177630, 177631) c6( ) c7(fq, ey); #c1(SNX24) c2(XP_005272029) c3(sorting nexin-24 isoform X2) c4(283666) c5( ) c6( ) c7(fq, ey); #c1(SNX24) c2(XP_006714655) c3(sorting nexin-24 isoform X5) c4(287940) c5(287941, 287942, 287943, 287944, 287945, 287946, 287947, 287948, 287949, 287950) c6( ) c7(fq, ey); #c1(SNX25) c2(XP_005263324) c3(sorting nexin-25 isoform X7) c4(278431) c5(278432, 278433) c6( ) c7(il, ha); #c1(SNX25) c2(XP_011530604) c3(sorting nexin-25 isoform X2) c4( ) c5(326719) c6( ) c7(il, ha); #c1(SNX25) c2(XP_011530606) c3(sorting nexin-25 isoform X4) c4(326720) c5( ) c6( ) c7(il, ha); #c1(SNX29) c2(NP_115543) c3(sorting nexin-29) c4(223137, 223138, 223139, 223140) c5(223141, 223142, 223143, 223144, 223145, 223146) c6( ) c7(oe, ac, pN); #c1(SNX29) c2(XP_005255740) c3(sorting nexin-29 isoform X11) c4( ) c5(273821, 273822, 273823, 273824, 273825, 273826, 273827, 273828, 273829, 273830, 273831, 273832, 273833, 273834, 273835, 273836, 273837, 273838, 273839, 273840, 273841, 273842, 273843, 273844, 273845, 273846, 273847, 273848) c6( ) c7(oe, ac, pN); #c1(SNX29) c2(XP_011521041) c3(sorting nexin-29 isoform X3) c4(316012) c5( ) c6( ) c7(oe, ac, pN); #c1(SNX29) c2(XP_011521042) c3(sorting nexin-29 isoform X4) c4(316013) c5( ) c6( ) c7(oe, ac, pN); #c1(SNX29) c2(XP_011521046) c3(sorting nexin-29 isoform X9) c4(316014) c5(316015, 316016, 316017, 316018, 316019, 316020) c6( ) c7(oe, ac, pN); #c1(SNX29) c2(XP_011521049) c3(sorting nexin-29 isoform X12) c4( ) c5(316021, 316022, 316023, 316024, 316025, 316026) c6( ) c7(oe, ac, pN); #c1(SNX3) c2(NP_001287857) c3(sorting nexin-3 isoform c) c4(113048) c5(113049, 113050, 113051, 113052, 113053, 113054) c6( ) c7(kj, arA, ff, bBU, cHS, en, QK); #c1(SNX3) c2(NP_001287858) c3(sorting nexin-3 isoform d) c4(113055) c5(113056, 113057, 113058, 113059, 113060, 113061) c6( ) c7(kj, arA, ff, bBU, cHS, en, QK); #c1(SNX3) c2(NP_003786) c3(sorting nexin-3 isoform a) c4(140790) c5(140791, 140792, 140793, 140794, 140795, 140796) c6( ) c7(kj, arA, ff, bBU, cHS, en, QK); #c1(SNX3) c2(NP_690040) c3(sorting nexin-3 isoform b) c4(244794) c5(244795, 244796, 244797, 244798, 244799, 244800) c6( ) c7(kj, arA, ff, bBU, cHS, en, QK); #c1(SNX30) c2(NP_001013012) c3(sorting nexin-30) c4(25436) c5(25437, 25438, 25439, 25440, 25441, 25442) c6( ) c7(Na, Nc); #c1(SNX30) c2(XP_005252043) c3(sorting nexin-30 isoform X2) c4( ) c5(271495, 271496, 271497, 271498, 271499, 271500) c6( ) c7(Na, Nc); #c1(SNX30) c2(XP_011516993) c3(sorting nexin-30 isoform X1) c4(310480) c5( ) c6( ) c7(Na, Nc); #c1(SNX5) c2(NP_689413) c3(sorting nexin-5 isoform a) c4(242678, 242679, 242680) c5(242681, 242682, 242683, 242684, 242685, 242686, 242687, 242688, 242689, 242690, 242691, 242692, 242693, 242694, 242695, 242696, 242697, 242698, 242699, 242700, 242701, 242702) c6( ) c7(af, aA); #c1(SNX9) c2(NP_057308) c3(sorting nexin-9) c4(190034, 190035) c5(190036, 190037, 190038, 190039, 190040, 190041, 190042, 190043, 190044, 190045) c6( ) c7(bM, bE, mQ, aT, bB, X); #c1(SNX9) c2(XP_005267072) c3(sorting nexin-9 isoform X1) c4( ) c5(280668) c6( ) c7(bM, bE, mQ, aT, bB, X); #c1(SNX9) c2(XP_011534188) c3(sorting nexin-9 isoform X2) c4( ) c5(330498, 330499, 330500, 330501, 330502) c6( ) c7(bM, bE, mQ, aT, bB, X); #c1(SOAT1) c2(NP_001239440) c3(sterol O-acyltransferase 1 isoform 2) c4( ) c5(88459, 88460, 88461, 88462, 88463, 88464) c6( ) c7(T, os, ea, e, fd, ci, amW, ip, x, aE, K, dQ, aVT, dH, aW, apn, bw, Qt, eM, nB, iT, qA, dD, akc, oY, aj, fr, ai, nV, zw, pe, kH, cU, h, eV, pi, aq, pm, iO, dB, hL, nL, B, bL, fM, dA, jx, eX, J, er, oT, nv, aWJ, aT, dz, jj, ae, af, iU, Y, cl, f, ck, sS, iM, dy, bt, zA, iW, rl, jh, aHv, oZ, eq, hF, ad, hd, ds, oW, pq, uQ, d, je, et, cr, rM, bz, ek, baa, pc, zr, aS, bC, y, kS); #c1(SOAT1) c2(NP_001239441) c3(sterol O-acyltransferase 1 isoform 3) c4( ) c5(88465, 88466, 88467, 88468, 88469) c6( ) c7(T, os, ea, e, fd, ci, amW, ip, x, aE, K, dQ, aVT, dH, aW, apn, bw, Qt, eM, nB, iT, qA, dD, akc, oY, aj, fr, ai, nV, zw, pe, kH, cU, h, eV, pi, aq, pm, iO, dB, hL, nL, B, bL, fM, dA, jx, eX, J, er, oT, nv, aWJ, aT, dz, jj, ae, af, iU, Y, cl, f, ck, sS, iM, dy, bt, zA, iW, rl, jh, aHv, oZ, eq, hF, ad, hd, ds, oW, pq, uQ, d, je, et, cr, rM, bz, ek, baa, pc, zr, aS, bC, y, kS); #c1(SOAT1) c2(XP_011508213) c3(sterol O-acyltransferase 1 isoform X1) c4(298803, 298804, 298805, 298806, 298807) c5(298808, 298809, 298810, 298811, 298812, 298813, 298814, 298815, 298816, 298817, 298818, 298819, 298820) c6( ) c7(T, os, ea, e, fd, ci, amW, ip, x, aE, K, dQ, aVT, dH, aW, apn, bw, Qt, eM, nB, iT, qA, dD, akc, oY, aj, fr, ai, nV, zw, pe, kH, cU, h, eV, pi, aq, pm, iO, dB, hL, nL, B, bL, fM, dA, jx, eX, J, er, oT, nv, aWJ, aT, dz, jj, ae, af, iU, Y, cl, f, ck, sS, iM, dy, bt, zA, iW, rl, jh, aHv, oZ, eq, hF, ad, hd, ds, oW, pq, uQ, d, je, et, cr, rM, bz, ek, baa, pc, zr, aS, bC, y, kS); #c1(SOAT1) c2(XP_011508214) c3(sterol O-acyltransferase 1 isoform X2) c4( ) c5(298821, 298822, 298823, 298824, 298825, 298826) c6( ) c7(T, os, ea, e, fd, ci, amW, ip, x, aE, K, dQ, aVT, dH, aW, apn, bw, Qt, eM, nB, iT, qA, dD, akc, oY, aj, fr, ai, nV, zw, pe, kH, cU, h, eV, pi, aq, pm, iO, dB, hL, nL, B, bL, fM, dA, jx, ex, J, er, oT, nv, aWJ, aT, dz, jj, ae, af, iU, Y, cl, f, ck, sS, iM, dy, bt, zA, iW, rl, jh, aHv, oZ, eq, hF, ad, hd, ds, oW, pq, uQ, d, je, et, cr, rM, bz, ek, baa, pc, zr, aS, bC, y, kS); #c1(SOAT2) c2(NP_003569) c3(sterol O-acyltransferase 2) c4(139233) c5(139234, 139235, 139236, 139237, 139238, 139239) c6( ) c7(iu, iV, l, dD, eR, u, cH, ea, at, aq, dL, j, ah, v, bq, dQ, eq, B); #c1(SOBP) c2(NP_060483) c3(sine oculis-binding protein homolog) c4(196195) c5(196196, 196197, 196198, 196199, 196200, 196201) c6( ) c7(gl, cHT, ac, nA); #c1(SOBP) c2(XP_011534223) c3(sine oculis-binding protein homolog isoform X3) c4(330577) c5( ) c6( ) c7(gl, cHT, ac, nA); #c1(SOBP) c2(XP_011534225) c3(sine oculis-binding protein homolog isoform X5) c4( ) c5(330578, 330579, 330580, 330581, 330582, 330583) c6( ) c7(gl, cHT, ac, nA); #c1(SOCS1) c2(NP_003736) c3(suppressor of cytokine signaling 1) c4(140526) c5(140527, 140528, 140529, 140530, 140531, 140532, 140533, 140534, 140535, 140536, 140537) c6( ) c7(bM, T, aC, ea, if, al, fd, bB, ci, aE, pX, K, eL, bc, cp, yo, pr, aW, hl, g, bw, Qt, eM, nB, cj, m, dD, wu, aw, aFc, n, aj, fr, qN, qW, eW, br, pe, kH, h, eV, dL, pi, A, mR, fJ, fQ, aq, pm, auC, iO, aOL, hs, hR, pg, eo, cB, B, cn, bL, fM, jp, fG, jx, eX, aA, v, C, AO, V, q, J, oT, aT, yu, dz, ae, af, iU, vT, nO, Y, r, lo, en, LV, t, ik, ck, iM, ig, bt, jh, eq, jE, ad, LH, hX, fU, hd, F, ds, lQ, wm, pq, NM, d, je, et, ff, cr, eZ, Ej, aF, hp, iV, bz, LB, gk, pc, bE, dO, hE, kS, y); #c1(SOCS2) c2(NP_001257400) c3(suppressor of cytokine signaling 2) c4(95307, 95308) c5(95309, 95310, 95311, 95312, 95313, 95314, 95315, 95316, 95317, 95318, 95319, 95320, 95321, 95322, 95323, 95324, 95325, 95326, 95327, 95328, 95329, 95330, 95331, 95332, 95333, 95334, 95335, 95336, 95337, 95338, 95339, 95340, 95341, 95342, 95343) c6( ) c7(bL, bM, T, Dw, aD, pQ, Fb, ci, C, JW, q, ad, cF, pe, vJ, aT, Ds, dz, ae, iU, et, aW, Y, fJ, ff, eZ, r, en, cl, pm, t, pc, hL, aS, m, y, ao); #c1(SOCS2) c2(XP_011537236) c3(suppressor of cytokine signaling 2 isoform X1) c4(334960, 334961) c5(334962, 334963, 334964, 334965, 334966) c6( ) c7(bL, bM, T, Dw, aD, pQ, Fb, ci, C, JW, q, ad, cF, pe, vJ, aT, Ds, dz, ae, iU, et, aW, Y, fJ, ff, eL, r, en, cl, pm, t, pc, hl, aS, m, y, ao); #c1(SOCS3) c2(NP_003946) c3(suppressor of cytokine signaling 3) c4(142377) c5(142378, 142379, 142380, 142381, 142382, 142383) c6( ) c7(bM, T, fR, ea, if, al, CH, bB, fd, ci, xE, aE, Ds, K, dF, cHU, pr, aW, hl, g, eE, ef, uC, cm, fT, eM, nB, aiM, U, Hb, dD, Bm, n, aj, fr, qN, ai, ke, jz, aZv, cU, IG, h, A, fJ, fe, aq, kp, pm, dB, bo, aOL, hL, hs, at, pg, eo, cB, B, bL, Dw, lH, jx, aA, v, j, C, V, q, J, eA, hW, aT, dz, bq, ae, kl, iU, nO, xR, Y, fO, r, lo, en, iK, ck, ig, dy, fD, bt, vi, gV, bjX, aCA, iW, dG, eq, ad, hX, fU, av, hd, F, ib, cHV, ds, ji, jo, d, Di, hG, et, yx, eH, ff, cr, eZ, aF, iV, KM, bz, gk, eU, pc, bE, aS, rH, ei, hE, y); #c1(SOCS4) c2(NP_543143) c3(suppressor of cytokine signaling 4) c4(233387) c5(233388, 233389, 233390, 233391, 233392, 233393, 233394, 233395, 233396, 233397, 233398, 233399, 233400, 233401) c6( ) c7(gk, F, bo, al, aOL, lG, v, dL, eL); #c1(SOCS5) c2(NP_659198) c3(suppressor of cytokine signaling 5) c4(239730) c5(239731, 239732, 239733, 239734, 239735, 239736, 239737, 239738, 239739, 239740) c6( ) c7(T, aCA, A, aOL, qR, at, fe, K, eE, qW); #c1(SOCS6) c2(XP_005266840) c3(suppressor of cytokine signaling 6 isoform X1) c4(280356) c5(280357, 280358, 280359, 280360, 280361, 280362) c6( ) c7(T, d, QO, Qu, eZ, r, aA, n, aq, aF, K, iR, q); #c1(SOCS7) c2(NP_055413) c3(suppressor of cytokine signaling 7) c4(180915) c5(180916, 180917, 180918, 180919, 180920, 180921, 180922, 180923, 180924, 180925) c6( ) c7(gk, T, d, F, Qu, bo, al, aOL, r, aA, n, aq, dL, v, aF, iR, eL, q); #c1(SOD1) c2(NP_000445) c3(superoxide dismutase [Cu-Zn]) c4(9064, 9065) c5(9066, 9067, 9068, 9069, 9070, 9071, 9072, 9073, 9074, 9075, 9076, 9077) c6( ) c7(ID, bM, T, xS, fR, aey, ea, al, bB, Vd, ci, qj, aR, Gt, aE, lu, Bc, bMh, dV, K, bU, hu, hH, wt, cHY, li, eG, zQ, aiV, apl, aW, cHX, wf, alR, hl, LZ, fc, dg, vs, arq, cnV, Ku, g, uC, cm, bb, eM, Kv, aem, wB, U, vy, bel, fC, nw, m, R, act, cZ, dt, dD, Ol, aWG, My, aw, n, cQ, aj, bOX, ct, nA, PJ, aHj, cd, co, Hx, b, no, i, ju, eT, Ak, h, lG, dL, bl, dd, awL, db, wn, wC, A, asp, acR, bYg, axR, fe, ax, ack, aq, wd, kk, aKv, hq, ar, W, bP, dB, at, oh, va, eo, acJ, eQ, B, L, bL, eR, mr, fG, rf, aA, cz, ti, aNG, j, il, V, q, J, eA, Kx, PW, di, MR, WY, HC, aT, kf, cy, aWJ, jj, bq, an, cHW, rz, ae, af, eu, alL, bA, ak, axW, WQ, r, TQ, aaE, HL, en, t, xw, f, bN, ck, bmS, rR, apq, tW, c, ac, cvr, wO, Uq, hr, wR, dy, AC, gT, aen, aya, cmC, bLx, wi, id, Pt, MH, qb, iW, jN, acU, dG, ah, eq, ad, BY, av, HF, F, Ae, aet, Hk, ts, oj, ab, oW, ama, ji, NM, sj, uX, d, lR, cH, PE, Zf, aav, ff, cr, eZ, kD, yy, bOW, aF, bz, dr, ek, lU, axl, gk, EW, akQ, VO, bE, dJ, aEY, aDB, aS, Kj, lM, y, kS, rk, pC); #c1(SOD2) c2(NP_001019636) c3(superoxide dismutase [Mn], mitochondrial isoform A precursor) c4(27797, 27798) c5(27799, 27800, 27801, 27802, 27803, 27804, 27805, 27806, 27807, 27808, 27809, 27810, 27811, 27812, 27813, 27814) c6( ) c7(bM, T, aC, fR, ea, e, al, bO, fd, fo, qj, aei, ci, HH, mF, aE, bW, azm, HR, K, oi, H, dQ, dF, hH, eL, aa, zQ, aW, fc, LZ, nY, vs, g, cnV, aBa, uC, cm, y, fT, bhB, eM, Kv, dc, mx, U, cj, vy, sv, aCy, uJ, m, abp, R, zv, iu, dD, gU, sw, Ol, aw, ht, NT, n, aj, fr, ct, qN, PJ, qW, cd, ai, ke, b, cc, h, lG, Gw, dL, vd, acd, awL, ahA, wC, A, mR, sp, aq, cv, ar, W, cA, bP, dB, hk, uw, bo, aic, bm, gw, at, eQ, lB, it, B, L, bL, fM, aCw, kA, yk, jx, aA, cR, ti, cz, hy, j, v, C, AE, bX, Zk, V, q, J, eA, ga, bgk, uT, lg, apJ, HC, aT, cy, bK, bq, cla, rz, an, ae, af, lr, bA, eu, ajU, ak, nO, Y, aM, r, akY, HL, en, t, kC, xw, f, ik, rY, ck, ac, c, pZ, mG, clb, hr, wR, dy, cS, cHZ, Gl, bmQ, MH, iW, jh, lD, aET, bS, dG, fu, eq, ad, kL, wD, fU, HF, av, ib, yt, aCJ, Hk, lC, ts, oW, lQ, jo, vl, sj, uQ, NP, uX, cH, ff, eZ, yy, So, gl, aF, iV, Z, bz, lU, bF, gk, axs, wz, gZ, aCl, aS, ei, Gu, i, Pk, X, rk, pC); #c1(SOD2) c2(NP_001019637) c3(superoxide dismutase [Mn], mitochondrial isoform B precursor) c4(27815) c5(27816, 27817, 27818, 27819, 27820, 27821, 27822, 27823, 27824, 27825) c6( ) c7(bM, T, aC, fR, ea, e, al, bO, fd, fo, qj, aei, ci, HH, mF, aE, bW, azm, HR, K, oi, H, dQ, dF, hH, eL, aa, zQ, aW, fc, LZ, nY, vs, g, cnV, aBa, uC, cm, y, fT, bhB, eM, Kv, dc, mx, U, cj, vy, sv, aCy, uJ, m, abp, R, zv, iu, dD, gU, sw, Ol, aw, ht, NT, n, aj, fr, ct, qN, PJ, qW, cd, ai, ke, b, cc, h, lG, Gw, dL, vd, acd, awL, ahA, wC, A, mR, sp, aq, cv, ar, W, cA, bP, dB, hk, uw, bo, aic, bm, gw, at, eQ, lB, it, B, L, bL, fM, aCw, kA, yk, jx, aA, cR, ti, cz, hy, j, v, C, AE, bX, Zk, V, q, J, eA, ga, bgk, uT, lg, apJ, HC, aT, cy, bK, bq, cla, rz, an, ae, af, lr, bA, eu, ajU, ak, nO, Y, aM, r, akY, HL, en, t, kC, xw, f, iK, rY, ck, ac, c, pZ, mG, clb, hr, wR, dy, cS, cHZ, Gl, bmQ, MH, iW, jh, lD, aET, bS, dG, fu, eq, ad, KL, wD, fU, HF, av, ib, yt, aCJ, Hk, lC, ts, oW, IQ, jo, vl, sj, uQ, NP, uX, cH, ff, eZ, yy, So, gl, aF, iV, Z, bz, lU, bF, gk, axs, wz, gZ, aCl, aS, ei, Gu, i, Pk, X, rk, pC); #c1(SOD3) c2(NP_003093) c3(extracellular superoxide dismutase [Cu-Zn] precursor) c4(135164) c5(135165, 135166, 135167, 135168, 135169, 135170) c6( ) c7(bL, sY, dD, gU, bmQ, DD, iW, e, aw, cz, BH, aj, j, qj, bX, PJ, eq, cd, ad, eA, Vp, hd, xE, uT, aE, aT, ab, cy, dL, K, rz, ae, NP, aW, sp, dN, cnV, t, nC, uC, qa, ek, LB, y, nB, dB, vZ, at, lC, rk, pC); #c1(SOHLH1) c2(NP_001012415) c3(spermatogenesis- and oogenesis-specific basic helix-loop-helix-containing protein 1 isoform b) c4(24980) c5(24981, 24982, 24983, 24984, 24985, 24986) c6( ) c7(vH, pt, aec); #c1(SOHLH1) c2(NP_001095147) c3(spermatogenesis- and oogenesis-specific basic helix-loop-helix-containing protein 1 isoform a) c4(40847) c5(40848, 40849, 40850, 40851, 40852, 40853) c6( ) c7(vH, pt, aec); #c1(SOHLH2) c2(NP_060296) c3(spermatogenesis- and oogenesis-specific basic helix-loop-helix-containing protein 2 isoform 1) c4(195245) c5(195246, 195247, 195248, 195249, 195250, 195251) c6( ) c7(il, t, T, zZ, m); #c1(SON) c2(NP_001278340) c3(protein SON isoform E) c4( ) c5(109554, 109555, 109556) c6( ) c7(ck, T, pa, ds, qK, aT); #c1(SON) c2(NP_115571) c3(protein SON isoform B) c4(223243) c5(223244, 223245, 223246, 223247, 223248, 223249, 223250, 223251, 223252, 223253, 223254, 223255, 223256, 223257, 223258, 223259, 223260, 223261) c6( ) c7(ck, T, pa, ds, qK, aT); #c1 (SON) c2(NP_620305) c3(protein SON isoform F) c4(237507, 237508) c5( ) c6( ) c7(ck, T, pa, ds, qK, aT); #c1(SORBS1) c2(NP_001030127) c3(sorbin and SH3 domain-containing protein 1 isoform 4) c4(30925) c5(30926, 30927, 30928, 30929, 30930, 30931, 30932, 30933, 30934, 30935) c6( ) c7(bL, bM, T, Ke, jx, cz, aj, v, q, J, eW, F, afn, aT, h, uV, kl, avR, aW, r, tv, aq, tu, avT, cT, rR, wg, ac, avS, tr, B); #c1(SORBS1) c2(NP_001030128) c3(sorbin and SH3 domain-containing protein 1 isoform 5) c4(30936) c5(30937, 30938, 30939, 30940, 30941, 30942, 30943, 30944, 30945, 30946) c6( ) c7(bL, bM, T, Ke, jx, cz, aj, v, q, J, eW, F, afn, aT, h, uV, kl, avR, aW, r, tv, aq, tu, avT, cT, rR, wg, ac, avS, tr, B); #c1(SORBS1) c2(NP_001277223) c3(sorbin and SH3 domain-containing protein 1 isoform 8) c4(109239) c5(109240, 109241, 109242, 109243, 109244, 109245) c6( ) c7(bL, bM, T, Ke, jx, cz, aj, v, q, J, eW, F, afn, aT, h, uV, kl, avR, aW, r, tv, aq, tu, avT, cT, rR, wg, ac, avS, tr, B); #c1(SORBS1) c2(NP_001277226) c3(sorbin and SH3 domain-containing protein 1 isoform 11) c4(109246) c5(109247, 109248, 109249, 109250, 109251, 109252) c6( ) c7(bL, bM, T, Ke, jx, cz, aj, v, q, J, eW, F, afn, aT, h, uV, kl, avR, aW, r, tv, aq, tu, avT, cT, rR, wg, ac, avS, tr, B); #c1(SORBS1) c2(NP_056200) c3(sorbin and SH3 domain-containing protein 1 isoform 2) c4(186286) c5(186287, 186288, 186289, 186290, 186291, 186292) c6( ) c7(bL, bM, T, Ke, jx, cz, aj, v, q, J, eW, F, afn, aT, h, uV, kl, avR, aW, r, tv, aq, tu, avT, cT, rR, wg, ac, avS, tr, B); #c1(SORBS1) c2(NP_079267) c3(sorbin and SH3 domain-containing protein 1 isoform 6) c4(217635) c5(217636, 217637, 217638, 217639, 217640, 217641) c6( ) c7(bL, bM, T, Ke, jx, cz, aj, v, q, J, eW, F, afn, aT, h, uV, kl, avR, aW, r, tv, aq, tu, avT, cT, rR, wg, ac, avS, tr, B); #c1(SORBS1) c2(XP_006717644) c3(sorbin and SH3 domain-containing protein 1 isoform X4) c4(291263) c5(291264, 291265, 291266, 291267, 291268, 291269, 291270, 291271, 291272, 291273) c6( ) c7(bL, bM, T, Ke, jx, cz, aj, v, q, J, eW, F, afn, aT, h, uV, kl, avR, aW, r, tv, aq, tu, avT, cT, rR, wg, ac, avS, tr, B); #c1(SORBS1) c2(XP_006717652) c3(sorbin and SH3 domain-containing protein 1 isoform X1) c4(291274) c5(291275, 291276, 291277, 291278, 291279, 291280, 291281, 291282, 291283, 291284) c6( ) c7(bL, bM, T, Ke, jx, cz, aj, v, q, J, eW, F, afn, aT, h, uV, kl, avR, aW, r, tv, aq, tu, avT, cT, rR, wg, ac, avS, tr, B); #c1(SORBS1) c2(XP_006717654) c3(sorbin and SH3 domain-containing protein 1 isoform X2) c4(291285) c5(291286, 291287, 291288, 291289, 291290, 291291) c6( ) c7(bL, bM, T, Ke, jx, cz, aj, v, q, J, eW, F, afn, aT, h, uV, kl, avR, aW, r, tv, aq, tu, avT, cT, rR, wg, ac, avS, tr, B); #c1(SORBS1) c2(XP_006717658) c3(sorbin and SH3 domain-containing protein 1 isoform X3) c4(291292) c5(291293, 291294, 291295, 291296, 291297, 291298) c6( ) c7(bL, bM, T, Ke, jx, cz, aj, v, q, J, eW, F, afn, aT, h, uV, kl, avR, aW, r, tv, aq, tu, avT, cT, rR, wg, ac, avS, tr, B); #c1(SORBS1) c2(XP_011537471) c3(sorbin and SH3 domain-containing protein 1 isoform X38) c4( ) c5(335081) c6( ) c7(bL, bM, T, Ke, jx, cz, aj, v, q, J, eW, F, afn, aT, h, uV, kl, avR, aW, r, tv, aq, tu, avT, cT, rR, wg, ac, avS, tr, B); #c1(SORBS2) c2(NP_001139142) c3(sorbin and SH3 domain-containing protein 2 isoform 3) c4( ) c5(58785, 58786, 58787, 58788, 58789, 58790, 58791, 58792, 58793, 58794, 58795, 58796, 58797, 58798, 58799, 58800, 58801, 58802, 58803, 58804, 58805, 58806, 58807, 58808, 58809, 58810, 58811, 58812, 58813, 58814, 58815, 58816, 58817, 58818, 58819, 58820, 58821, 58822, 58823, 58824, 58825, 58826, 58827) c6( ) c7(T, aC, aE, it); #c1(SORBS2) c2(NP_001139143) c3(sorbin and SH3 domain-containing protein 2 isoform 4) c4( ) c5(58828, 58829, 58830, 58831, 58832) c6( ) c7(T, aC, aE, it); #c1(SORBS2) c2(NP_001139144) c3(sorbin and SH3 domain-containing protein 2 isoform 5) c4(58833) c5(58834, 58835, 58836, 58837, 58838, 58839) c6( ) c7(T, aC, aE, it); #c1(SORBS2) c2(NP_001139145) c3(sorbin and SH3 domain-containing protein 2 isoform 6) c4( ) c5(58840, 58841, 58842, 58843, 58844) c6( ) c7(T, aC, aE, it); #c1(SORBS2) c2(NP_001139146) c3(sorbin and SH3 domain-containing protein 2 isoform 7) c4(58845, 58846) c5(58847, 58848, 58849, 58850, 58851, 58852, 58853, 58854, 58855, 58856, 58857, 58858) c6( ) c7(T, aC, aE, it); #c1(SORBS2) c2(NP_001139147) c3(sorbin and SH3 domain-containing protein 2 isoform 8) c4( ) c5(58859) c6( ) c7(T, aC, aE, it); #c1(SORBS2) c2(NP_001257700) c3(sorbin and SH3 domain-containing protein 2 isoform 9) c4(95586, 95587) c5(95588, 95589, 95590, 95591, 95592, 95593, 95594, 95595, 95596, 95597, 95598, 95599, 95600, 95601, 95602, 95603) c6( ) c7(T, aC, aE, it); #c1(SORBS2) c2(NP_003594) c3(sorbin and SH3 domain-containing protein 2 isoform 1) c4(139443) c5(139444, 139445, 139446, 139447, 139448, 139449, 139450, 139451, 139452, 139453) c6( ) c7(T, aC, aE, it); #c1(SORBS2) c2(XP_005263364) c3(sorbin and SH3 domain-containing protein 2 isoform X20) c4(278455) c5(278456) c6( ) c7(T, aC, aE, it); #c1(SORBS2) c2(XP_005263365) c3(sorbin and SH3 domain-containing protein 2 isoform X28) c4(278457) c5(278458) c6( ) c7(T, aC, aE, it); #c1(SORBS2) c2(XP_005263367) c3(sorbin and SH3 domain-containing protein 2 isoform X30) c4( ) c5(278459) c6( ) c7(T, aC, aE, it); #c1(SORBS2) c2(XP_005263370) c3(sorbin and SH3 domain-containing protein 2 isoform X33) c4(278460) c5(278461) c6( ) c7(T, aC, aE, it); #c1(SORBS2) c2(XP_006714444) c3(sorbin and SH3 domain-containing protein 2 isoform X25) c4(287843) c5( ) c6( ) c7(T, aC, aE, it); #c1(SORBS2) c2(XP_011530665) c3(sorbin and SH3 domain-containing protein 2 isoform X2) c4( ) c5(326745, 326746, 326747, 326748, 326749) c6( ) c7(T, aC, aE, it); #c1(SORBS2) c2(XP_011530667) c3(sorbin and SH3 domain-containing protein 2 isoform X5) c4(326750, 326751) c5( ) c6( ) c7(T, aC, aE, it); #c1(SORBS2) c2(XP_011530668) c3(sorbin and SH3 domain-containing protein 2 isoform X6) c4(326752) c5( ) c6( ) c7(T, aC, aE, it); #c1(SORBS2) c2(XP_011530676) c3(sorbin and SH3 domain-containing protein 2 isoform X12) c4(326753, 326754, 326755) c5( ) c6( ) c7(T, aC, aE, it); #c1(SORBS3) c2(NP_005766) c3(vinexin isoform 1) c4(159160) c5(159161, 159162, 159163, 159164, 159165, 159166) c6( ) c7(oe); #c1(SORBS3) c2(XP_005273428) c3(vinexin isoform X1) c4( ) c5(284583, 284584, 284585, 284586, 284587) c6( ) c7(oe); #c1(SORBS3) c2(XP_006716329) c3 (vinexin isoform X2) c4(290226) c5( ) c6( ) c7(oe); #c1(SORBS3) c2(XP_006716331) c3(vinexin isoform X3) c4( ) c5(290227) c6( ) c7(oe); #c1(SORCS1) c2(NP_001013049) c3(VPS10 domain-containing receptor SorCS1 isoform b precursor) c4(25443) c5(25444, 25445, 25446, 25447, 25448, 25449) c6( ) c7(bL, cF, dt, dD, A, oe, bo, aj, v, dy, eq, an, af, J); #c1(SORCS1) c2(NP_001193501) c3(VPS10 domain-containing receptor SorCS1 isoform f precursor) c4(82239) c5(82240, 82241, 82242, 82243, 82244, 82245) c6( ) c7(bL, cF, dt, dD, A, oe, bo, aj, v, dy, eq, an, af, J); #c1(SORCS1) c2(NP_443150) c3(VPS10 domain-containing receptor SorCS1 isoform a precursor) c4(229656) c5(229657, 229658, 229659, 229660, 229661, 229662) c6( ) c7(bL, cF, dt, dD, A, oe, bo, aj, v, dy, eq, an, af, J); #c1(SORCS1) c2(XP_011537505) c3(VPS10 domain-containing receptor SorCS1 isoform X5) c4(335113) c5( ) c6( ) c7(bL, cF, dt, dD, A, De, bo, aj, v, dy, eq, an, af, J); #c1(SORCS1) c2(XP_011537509) c3(VPS10 domain-containing receptor SorCS1 isoform X9) c4( ) c5(335114, 335115, 335116, 335117, 335118, 335119) c6( ) c7(bL, cF, dt, dD, A, oe, bo, aj, v, dy, eq, an, af, J); #c1(SORCS2) c2(NP_065828) c3(VPS10 domain-containing receptor SorCS2 precursor) c4(205706) c5(205707, 205708, 205709, 205710, 205711, 205712) c6( ) c7(bL, ac, gZ, j, bK, ad); #c1(SORCS2) c2(XP_005248044) c3(VPS10 domain-containing receptor SorCS2 isoform X1) c4( ) c5(268181) c6( ) c7(bL, ac, gZ, j, bK, ad); #c1(SORCS2) c2(XP_011511819) c3(VPS10 domain-containing receptor SorCS2 isoform X5) c4(302490) c5(302491, 302492, 302493, 302494, 302495) c6( ) c7(bL, ac, gZ, j, bK, ad); #c1(SORCS3) c2(NP_055793) c3(VPS10 domain-containing receptor SorCS3 precursor) c4(183540, 183541) c5(183542, 183543, 183544, 183545, 183546, 183547, 183548) c6( ) c7(Z, bS, j, ct, aA); #c1(SORCS3) c2(XP_011537844) c3(VPS10 domain-containing receptor SorCS3 isoform X2) c4(335352) c5(335353, 335354, 335355, 335356, 335357, 335358, 335359, 335360, 335361, 335362, 335363) c6( ) c7(Z, bS, j, ct, aA); #c1(SORD) c2(NP_003095) c3(sorbitol dehydrogenase) c4(135171) c5(135172, 135173, 135174, 135175, 135176, 135177) c6( ) c7(bL, T, s, Pq, Bc, vy, wz, aj, dy, bq, t, J); #c1(SORL1) c2(NP_003096) c3(sortilin-related receptor preproprotein) c4(135178) c5(135179, 135180, 135181, 135182, 135183, 135184, 135185) c6( ) c7(bL, dD, aaH, ea, oe, cz, aj, eq, hF, fK, J, x, sO, bk, af, cm, f, ac, aS, k, bt, CW, L); #c1(SOS1) c2(NP_005624) c3(son of sevenless homolog 1) c4(158036, 158037) c5(158038, 158039, 158040, 158041, 158042, 158043, 158044, 158045, 158046, 158047, 158048, 158049, 158050, 158051, 158052, 158053) c6( ) c7(T, apr, yS, ea, if, pf, al, bll, fd, bB, axk, ci, bv, aE, K, eL, bc, cp, hm, bvR, aW, fc, Az, PQ, brr, g, eF, qJ, cnj, eM, iT, U, Hb, m, dD, Ol, aw, NT, n, aj, fr, qN, md, afR, ft, cc, zw, Nn, gd, eV, dL, bl, ux, Ha, cC, blP, fe, aq, yq, fq, iO, Sc, hk, Pl, bjk, bD, bli, B, bL, AS, eR, fG, jx, eX, aA, fF, v, yO, q, J, er, eA, aup, nX, afU, aT, cxs, cy, aut, bK, jj, bau, ae, dl, jf, Uf, Sh, Qr, nO, iU, Y, ev, r, aYp, en, aDY, t, nC, ik, pb, bN, ck, rR, nM, QG, c, hc, bt, ao, hS, On, kh, ha, atS, VT, jh, wV, eq, iJ, ad, dP, fU, hd, ib, MS, py, pq, d, ff, eH, eZ, nf, aF, KM, bz, dr, ek, bF, gk, KD, clc, bE, lF, Hs, cP, hE, y); #c1(SOS1) c2(XP_005264572) c3(son of sevenless homolog 1 isoform X5) c4(279087, 279088) c5(279089, 279090, 279091, 279092, 279093, 279094, 279095, 279096, 279097, 279098, 279099, 279100) c6( ) c7(T, apr, yS, ea, if, pf, al, bll, fd, bB, axk, ci, bv, aE, K, eL, bc, cp, hm, bvR, aW, fc, Az, PQ, brr, g, eF, qJ, cnj, eM, iT, U, Hb, m, dD, Ol, aw, NT, n, aj, fr, qN, md, afR, ft, cc, zw, Nn, gd, eV, dL, bl, ux, Ha, cC, blP, fe, aq, yq, fq, iO, Sc, hk, Pl, bjk, bD, bli, B, bL, AS, eR, fG, jx, eX, aA, fF, v, yO, q, J, er, eA, aup, nX, afU, aT, cxs, cy, aut, bK, jj, bau, ae, dl, jf, Uf, Sh, Ar, no, iU, Y, ev, r, aYp, en, aDY, t, nC, ik, pb, bN, ck, rR, nM, QG, c, hc, bt, ao, hS, On, kh, ha, atS, VT, jh, wV, eq, iJ, ad, dP, fU, hd, ib, MS, py, pq, d, ff, eH, eZ, nf, af, KM, bz, dr, ek, bF, gk, KD, clc, bE, lF, Hs, cP, hE, y); #c1(SOS1) c2(XP_011531364) c3(son of sevenless homolog 1 isoform X3) c4(327367) c5( ) c6( ) c7(T, apr, yS, ea, if, pf, al, bll, fd, bB, axk, ci, bv, aE, K, eL, bc, cp, hm, bvR, aW, fc, Az, PQ, brr, g, eF, qJ, cnj, eM, iT, U, Hb, m, dD, Ol, aw, NT, n, aj, fr, qN, md, afR, ft, cc, zw, Nn, gd, eV, dL, bl, ux, Ha, cC, blP, fe, aq, yq, fq, iO, Sc, hk, Pl, bjk, bD, bli, B, bL, AS, eR, fG, jx, eX, aA, fF, v, yO, q, J, er, eA, aup, nX, afU, aT, cxs, cy, aut, bK, jj, bau, ae, dl, jf, Uf, Sh, Qr, nO, iU, Y, ev, r, aYp, en, aDY, t, nC, ik, pb, bN, ck, rR, nM, QG, c, hc, bt, ao, hS, On, kh, ha, atS, VT, jh, wV, eq, iJ, ad, dP, fU, hd, ib, MS, py, pq, d, ff, eH, eZ, nf, aF, KM, bz, dr, ek, bF, gk, KD, clc, bE, lF, Hs, cP, hE, y); #c1(SOS1) c2(XP_011531367) c3(son of sevenless homolog 1 isoform X7) c4(327368) c5(327369, 327370, 327371, 327372, 327373, 327374) c6( ) c7(T, apr, yS, ea, if, pf, al, bll, fd, bB, axk, ci, bv, aE, K, eL, bc, cp, hm, bvR, aW, fc, Az, PQ, brr, g, eF, qJ, cnj, eM, iT, U, Hb, m, dD, Ol, aw, NT, n, aj, fr, qN, md, afR, ft, cc, zw, Nn, gd, eV, dL, bl, ux, Ha, cC, blP, fe, aq, yq, fq, iO, Sc, hk, Pl, bjk, bD, bli, B, bL, AS, eR, fG, jx, eX, aA, fF, v, yO, q, J, er, eA, aup, nX, afU, aT, cxs, cy, aut, bK, jj, bau, ae, dl, jf, Uf, Sh, Ar, nO, iU, Y, ev, r, aYp, en, aDY, t, nC, iK, pb, bN, ck, rR, nM, QG, c, hc, bt, ao, hS, On, kh, ha, atS, VT, jh, wV, eq, iJ, ad, dP, fU, hd, ib, MS, py, pq, d, ff, eH, eZ, nf, aF, KM, bz, dr, ek, bF, gk, KD, clc, bE, lF, Hs, cP, hE, y); #c1(SOS2) c2(NP_008870) c3(son of sevenless homolog 2) c4(169346, 169347) c5(169348, 169349, 169350, 169351, 169352, 169353, 169354, 169355, 169356, 169357, 169358, 169359) c6( ) c7(T, dD, yS, Ol, ea, ha, jh, wV, eq, eA, ad, afR, aE, vJ, afU, aT, bk, K, ux, ae, af, cC, d, aW, Y, nf, t, nC, bN, bF, cnj, c, eM, iT, U, lF, y, bt); #c1(SOST) c2(NP_079513) c3(sclerostin precursor) c4(218495) c5(218496, 218497, 218498, 218499, 218500, 218501) c6( ) c7(T, cle, cld, zs, fd, clf, v, zU, sg, ad, zw, bfJ, caw, rv, bA, eH, eF, akb, aSb, dr, D, bi, it); #c1(SOSTDC1) c2(NP_056279) c3(sclerostin domain-containing protein 1 precursor) c4(186907, 186908) c5(186909, 186910, 186911, 186912, 186913, 186914, 186915, 186916, 186917, 186918, 186919, 186920, 186921, 186922, 186923, 186924) c6(186925, 186926, 186927, 186928) c7(es, T, py, iA, aW, aT, dy, ci, fr, er); #c1(SOX1) c2(NP_005977) c3(transcription factor SOX-1) c4(160939) c5(160940, 160941, 160942, 160943, 160944, 160945) c6( ) c7(xC, ft, if, m, qN, t, B, ad); #c1(SOX10) c2(NP_008872) c3 (transcription factor SOX-10) c4(169378, 169379, 169380) c5(169381, 169382, 169383, 169384, 169385, 169386, 169387, 169388, 169389, 169390, 169391, 169392, 169393, 169394, 169395, 169396, 169397, 169398, 169399, 169400, 169401, 169402, 169403, 169404) c6( ) c7(cZ, T, atY, agU, amj, ci, bjN, clh, bX, clg, iJ, eA, aR, yn, aJe, aJf, asG, KN, aT, hP, clk, bu, dd, cb, ahE, jf, UL, aW, Y, aek, Ls, ahl, as, aTa, cm, fs, cfO, Hp, ck, bgy, dB, hk, mG, cli, Km, clj, B); #c1(SOX11) c2(NP_003099) c3(transcription factor SOX-11) c4(135300) c5(135301, 135302, 135303, 135304, 135305, 135306, 135307) c6( ) c7(T, gU, fM, oe, jh, aA, fd, n, Ea, cd, eA, zn, x, ft, qK, hn, dL, an, eL, jf, d, kj, af, as, hf, t, bz, fq, ck, kn, ac, dy, m, k, oQ, bD); #c1(SOX12) c2(NP_008874) c3(transcription factor SOX-12) c4(169417) c5(169418, 169419, 169420, 169421, 169422, 169423, 169424, 169425, 169426, 169427, 169428) c6( ) c7(bB, bE); #c1(SOX13) c2(NP_005677) c3(transcription factor SOX-13) c4(158444, 158445, 158446) c5(158447, 158448, 158449, 158450, 158451, 158452) c6( ) c7(bL, A, yG, cz, aj, J); #c1(SOX13) c2(XP_005245680) c3(transcription factor SOX-13 isoform X1) c4( ) c5(266889, 266890, 266891, 266892, 266893, 266894, 266895, 266896, 266897, 266898, 266899, 266900) c6( ) c7(bL, A, yG, cz, aj, J); #c1(SOX14) c2(NP_004180) c3(transcription factor SOX-14) c4(144135) c5(144136, 144137, 144138, 144139, 144140, 144141) c6( ) c7(anB, Km, hl, arB); #c1(SOX15) c2(NP_008873) c3(protein SOX-15) c4(169405) c5(169406, 169407, 169408, 169409, 169410, 169411, 169412, 169413, 169414, 169415, 169416) c6( ) c7(T, kl, aC, aE, bE, bB, ey); #c1(SOX17) c2(NP_071899) c3(transcription factor SOX-17) c4(212156) c5(212157, 212158, 212159, 212160, 212161, 212162) c6( ) c7(T, wu, ht, aA, bB, q, jz, tT, aT, Vv, wm, hu, de, d, aW, r, aF, apx, cm, bz, cll, eM, bE, eD, gX, KY, it, us); #c1(SOX18) c2(NP_060889) c3(transcription factor SOX-18) c4(198417) c5(198418, 198419, 198420, 198421, 198422, 198423, 198424) c6( ) c7(Ck, T, dD, alu, aW, ea, clm, aT, Vv, n, aA, aF, eq, lQ, Pk); #c1(SOX2) c2(NP_003097) c3(transcription factor SOX-2) c4(135186) c5(135187, 135188, 135189, 135190, 135191, 135192, 135193, 135194, 135195, 135196, 135197, 135198, 135199, 135200, 135201, 135202, 135203, 135204, 135205, 135206, 135207, 135208, 135209, 135210, 135211, 135212, 135213, 135214, 135215, 135216, 135217, 135218, 135219, 135220, 135221, 135222, 135223, 135224, 135225, 135226, 135227, 135228, 135229, 135230, 135231, 135232, 135233, 135234, 135235, 135236, 135237, 135238, 135239, 135240, 135241, 135242, 135243, 135244, 135245, 135246, 135247, 135248, 135249, 135250, 135251, 135252, 135253, 135254, 135255, 135256, 135257, 135258, 135259, 135260, 135261, 135262, 135263, 135264, 135265, 135266, 135267, 135268, 135269, 135270, 135271, 135272, 135273, 135274, 135275, 135276, 135277, 135278, 135279, 135280, 135281, 135282, 135283, 135284, 135285, 135286) c6(135287) c7(T, acz, if, fd, bB, ci, beM, aE, hu, hm, Jl, aW, az, g, as, eF, cm, eM, m, avU, gD, wu, ht, n, aj, bBS, qN, Bl, ft, A, kj, AQ, Lh, mo, auC, kn, W, dB, Qu, eD, Be, hR, it, B, dA, eR, aA, v, il, AO, q, J, eA, aT, rO, bq, ahE, ae, jf, s, vT, oV, Y, r, ahl, en, t, KQ, ik, mG, hO, Da, UF, Bd, rA, KY, ha, Gn, ad, dP, av, wm, cnt, d, ff, eH, eZ, aF, iV, bz, Hp, bE, Km, bkh, oQ, iu); #c1(SOX21) c2(NP_009015) c3 (transcription factor SOX-21) c4(170252) c5(170253, 170254, 170255, 170256, 170257, 170258, 170259) c6( ) c7(eA, ft, d, dr, pr); #c1(SOX3) c2(NP_005625) c3(transcription factor SOX-3) c4(158054) c5(158055, 158056, 158057, 158058, 158059, 158060, 158061) c6( ) c7(bBJ, gU, clq, acz, ml, it, clp, n, adi, v, nA, afz, cd, acX, ahy, ft, mh, RN, tT, xW, vH, EL, an, af, jf, aeT, avP, cln, OX, Rj, nf, Pd, dr, EJ, clo); #c1(SOX4) c2(NP_003098) c3(transcription factor SOX-4) c4(135288) c5(135289, 135290, 135291, 135292, 135293, 135294, 135295, 135296, 135297, 135298, 135299) c6( ) c7(T, dA, jp, aA, n, kM, q, ad, eA, iC, av, x, ft, aE, qK, aT, dL, K, ae, eL, cp, bA, jf, d, je, aW, Y, r, kp, aF, g, cl, cM, bz, ck, iM, kP, Dy, eM, dB, bE, iT, hl, k, bD, bt, B); #c1(SOX5) c2(NP_001248343) c3(transcription factor SOX-5 isoform d) c4(93731) c5(93732, 93733, 93734, 93735, 93736, 93737, 93738, 93739, 93740, 93741) c6( ) c7(ae, iQ, VX, ND, pN, mr, wu, wL, Kr, vH, amj, at, eA, wm, pt, fq, pb, ad); #c1(SOX5) c2(NP_008871) c3(transcription factor SOX-5 isoform a) c4(169360) c5(169361, 169362, 169363, 169364, 169365, 169366, 169367, 169368, 169369, 169370, 169371, 169372, 169373, 169374, 169375, 169376, 169377) c6( ) c7(ae, iQ, VX, ND, pN, mr, wu, wL, Kr, vH, amj, at, eA, wm, pt, fq, pb, ad); #c1(SOX5) c2(NP_694534) c3(transcription factor SOX-5 isoform b) c4(245086) c5( ) c6( ) c7(ae, iQ, VX, ND, pN, mr, wu, wL, Kr, vH, amj, at, eA, wm, pt, fq, pb, ad); #c1(SOX5) c2(NP_821078) c3(transcription factor SOX-5 isoform c) c4( ) c5(253208, 253209, 253210, 253211, 253212, 253213) c6( ) c7(ae, iQ, VX, ND, pN, mr, wu, wL, Kr, vH, amj, at, eA, wm, pt, fq, pb, ad); #c1(SOX5) c2(XP_011519141) c3(transcription factor SOX-5 isoform X7) c4(313733, 313734) c5(313735, 313736, 313737, 313738, 313739, 313740) c6( ) c7(ae, iQ, VX, ND, pN, mr, wu, wL, Kr, vH, amj, at, eA, wm, pt, fq, pb, ad); #c1(SOX6) c2(NP_001139283) c3(transcription factor SOX-6 isoform 3) c4(59112, 59113, 59114) c5(59115, 59116, 59117, 59118, 59119, 59120, 59121, 59122, 59123, 59124, 59125, 59126, 59127, 59128, 59129) c6( ) c7(mj, dY, aw, ht, v, eA, hu, it, B, zV, bA); #c1(SOX6) c2(NP_001139291) c3(transcription factor SOX-6 isoform 4) c4(59137) c5(59138, 59139, 59140, 59141, 59142, 59143, 59144, 59145, 59146, 59147, 59148) c6( ) c7(mj, dY, aw, ht, v, eA, hu, it, B, zV, bA); #c1(SOX6) c2(NP_059978) c3(transcription factor SOX-6 isoform 1) c4(193436, 193437) c5(193438, 193439, 193440, 193441, 193442, 193443, 193444, 193445, 193446, 193447, 193448, 193449, 193450, 193451) c6( ) c7(mj, dY, aw, ht, v, eA, hu, it, B, zV, bA); #c1(SOX6) c2(NP_201583) c3(transcription factor SOX-6 isoform 2) c4(228287) c5(228288, 228289, 228290, 228291, 228292, 228293, 228294, 228295, 228296, 228297) c6( ) c7(mj, dY, aw, ht, v, eA, hu, it, B, zV, bA); #c1(SOX7) c2(NP_113627) c3(transcription factor SOX-7) c4(221792, 221793) c5(221794, 221795, 221796, 221797, 221798, 221799, 221800, 221801, 221802, 221803, 221804, 221805, 221806, 221807, 221808, 221809) c6( ) c7(md, T, eM, aW, wk, hL, aT, r, n, aA, aF, q, g, cl, bD, bz, B, ad); #c1(SOX8) c2(NP_055402) c3 (transcription factor SOX-8) c4(180844) c5(180845, 180846, 180847, 180848, 180849, 180850) c6( ) c7(WH, kj, pr, fd, bX, V, aMi); #c1(SOX9) c2(NP_000337) c3(transcription factor SOX-9) c4(7383, 7384) c5(7385, 7386, 7387, 7388, 7389, 7390, 7391, 7392, 7393, 7394, 7395, 7396, 7397) c6( ) c7(bM, T, aC, pf, clt, bB, ci, bjN, aR, Na, aE, aeO, Eo, Pl, cls, hu, eL, UL, zQ, aeR, bgA, aGb, aW, aeC, pr, Az, nY, g, eM, BP, apy, m, Le, ht, n, aj, uE, zP, cd, FN, bff, UD, dL, jy, aMi, Pp, kp, acD, dB, hk, aNd, ln, bD, aA, aNe, q, eA, aeZ, afQ, aT, clk, jj, ae, jf, s, Y, aek, r, en, cl, t, ck, cgp, Kg, kh, clr, acl, axd, adW, ad, iC, vJ, hP, lQ, aeT, d, VX, ff, cr, aF, aiz, bz, dr, Ew, bE, X, kS, iu); #c1(SP1) c2(NP_612482) c3(transcription factor Sp1 isoform a) c4(236621) c5(236622, 236623, 236624, 236625, 236626, 236627, 236628) c6( ) c7(bM, T, dD, fR, ea, if, aw, ha, fd, aA, lj, cz, hy, dG, bB, ci, eq, qN, lR, akG, q, ad, eA, hX, eW, bv, Hx, gJ, av, aE, ec, iu, aT, ab, cy, hD, dL, jy, K, h, vx, ae, af, cp, bA, d, BN, B, s, Jl, aW, Y, aiV, nO, fc, eZ, r, ff, aq, af, en, t, bz, pb, bN, ek, ck, bP, eM, nB, bE, hk, VA, X, wB, uz, np, k, m, cP, nz, it, ne, ao, cn); #c1(SP1) c2(XP_011536998) c3(transcription factor Sp1 isoform X1) c4(334744) c5(334745, 334746, 334747, 334748, 334749, 334750) c6( ) c7(bM, T, dD, fR, ea, if, aw, ha, fd, aA, lj, cz, hy, dG, bB, ci, eq, qN, lR, akG, q, ad, eA, hX, eW, bv, Hx, gJ, av, aE, ec, iu, aT, ab, cy, hD, dL, jy, K, h, vx, ae, af, cp, bA, d, BN, B, s, Jl, aW, Y, aiV, nO, fc, eZ, r, ff, aq, aF, en, t, bz, pb, bN, ek, ck, bP, eM, nB, bE, hk, VA, X, wB, uz, np, k, m, cP, nz, it, ne, ao, cn); #c1(SP100) c2(NP_001073860) c3(nuclear autoantigen Sp-100 isoform 1) c4(36138, 36139) c5(36140, 36141, 36142, 36143, 36144, 36145, 36146, 36147, 36148, 36149, 36150, 36151, 36152) c6( ) c7(ck, eW, T, pi, d, cF, dB, cj, lo, eA, jj, kS, bc, ip); #c1(SP100) c2(NP_001193630) c3(nuclear autoantigen Sp-100 isoform 3) c4(82379) c5(82380, 82381, 82382, 82383, 82384, 82385) c6( ) c7(ck, eW, T, pi, d, cF, dB, cj, lo, eA, jj, kS, bc, ip); #c1(SP100) c2(NP_001193631) c3(nuclear autoantigen Sp-100 isoform 4) c4( ) c5(82386, 82387, 82388, 82389, 82390, 82391, 82392, 82393, 82394, 82395, 82396, 82397) c6( ) c7(ck, eW, T, pi, d, cF, dB, cj, lo, eA, jj, kS, bc, ip); #c1(SP100) c2(NP_001193632) c3(nuclear autoantigen Sp-100 isoform 5) c4(82398) c5(82399, 82400, 82401, 82402, 82403, 82404) c6( ) c7(ck, eW, T, pi, d, cF, dB, cj, lo, eA, jj, kS, bc, ip); #c1(SP100) c2(NP_001193633) c3(nuclear autoantigen Sp-100 isoform 6) c4(82405) c5(82406, 82407, 82408, 82409, 82410, 82411) c6( ) c7(ck, eW, T, pi, d, cF, dB, cj, lo, eA, jj, kS, bc, ip); #c1(SP100) c2(NP_003104) c3(nuclear autoantigen Sp-100 isoform 2) c4(135353, 135354, 135355) c5(135356, 135357, 135358, 135359, 135360, 135361, 135362) c6( ) c7(ck, eW, T, pi, d, cF, dB, cj, lo, eA, jj, kS, bc, ip); #c1(SP110) c2(NP_001171944) c3(sp110 nuclear body protein isoform d) c4(72067, 72068, 72069, 72070, 72071) c5(72072, 72073, 72074, 72075, 72076, 72077, 72078, 72079, 72080, 72081, 72082, 72083, 72084, 72085, 72086, 72087, 72088, 72089, 72090, 72091, 72092, 72093, 72094, 72095, 72096, 72097) c6( ) c7(dA, fR, ig, oV, jx, cj, csx, aCK, LH); #c1(SP110) c2(NP_004501) c3(sp110 nuclear body protein isoform b) c4( ) c5(146966, 146967, 146968, 146969, 146970, 146971, 146972, 146973, 146974, 146975) c6( ) c7(dA, fR, ig, oV, jx, cj, csx, aCK, LH); #c1(SP110) c2(XP_005246582) c3(sp110 nuclear body protein isoform X3) c4(267244) c5(267245, 267246, 267247, 267248, 267249, 267250) c6( ) c7(dA, fR, ig, oV, jx, cj, csx, aCK, LH); #c1(SP110) c2(XP_006712550) c3(sp110 nuclear body protein isoform X4) c4(286721) c5(286722, 286723, 286724, 286725, 286726, 286727) c6( ) c7(dA, fR, ig, oV, jx, cj, csx, aCK, LH); #c1(SP110) c2(XP_006712552) c3(sp110 nuclear body protein isoform X7) c4(286728) c5( ) c6( ) c7(dA, fR, ig, oV, jx, cj, csx, aCK, LH); #c1(SP140) c2(NP_001005176) c3(nuclear body protein SP140 isoform 2) c4(21106) c5(21107, 21108, 21109, 21110, 21111, 21112) c6( ) c7(FR, cj, lo, jj, lf, V, bc); #c1(SP140) c2(NP_001265380) c3(nuclear body protein SP140 isoform 3) c4(98399) c5(98400, 98401, 98402, 98403, 98404, 98405) c6( ) c7(fR, cj, lo, jj, lf, V, bc); #c1(SP140) c2(NP_001265382) c3(nuclear body protein SP140 isoform 5) c4(98406) c5(98407, 98408, 98409, 98410, 98411, 98412) c6( ) c7(fR, cj, lo, jj, lf, V, bc); #c1(SP140) c2(NP_009168) c3(nuclear body protein SP140 isoform 1) c4(171235) c5(171236, 171237, 171238, 171239, 171240, 171241) c6( ) c7(fR, cj, lo, jj, lf, V, bc); #c1(SP140) c2(XP_005246311) c3(nuclear body protein SP140 isoform X9) c4(267037) c5(267038, 267039, 267040, 267041, 267042, 267043) c6( ) c7(FR, cj, lo, jj, lf, V, bc); #c1(SP140) c2(XP_011508823) c3(nuclear body protein SP140 isoform X12) c4(299404) c5(299405, 299406, 299407, 299408, 299409, 299410) c6( ) c7(fR, cj, lo, jj, lf, V, bc); #c1(SP140) c2(XP_011508825) c3(nuclear body protein SP140 isoform X14) c4(299411) c5(299412, 299413, 299414, 299415, 299416, 299417) c6( ) c7(fR, cj, lo, jj, lf, V, bc); #c1(SP2) c2(NP_003101) c3(transcription factor Sp2) c4(135308) c5(135309, 135310, 135311, 135312, 135313, 135314, 135315, 135316) c6( ) c7(ff, jE, en); #c1(SP2) c2(XP_011523442) c3(transcription factor Sp2 isoform X5) c4(317971) c5( ) c6( ) c7(ff, jE, en); #c1(SP2) c2(XP_011523446) c3(transcription factor Sp2 isoform X8) c4( ) c5(317972, 317973, 317974, 317975, 317976) c6( ) c7(ff, jE, en); #c1(SP3) c2(NP_001017371) c3(transcription factor Sp3 isoform 2) c4( ) c5(26368, 26369, 26370, 26371, 26372) c6( ) c7(bv, gJ, hW, aw, at, gl, dL, dy, eA, V, B); #c1(SP3) c2(NP_001166183) c3 (transcription factor Sp3 isoform 3) c4(68469, 68470) c5( ) c6( ) c7(bv, gJ, hW, aw, at, gl, dL, dy, eA, V, B); #c1(SP3) c2(NP_003102) c3(transcription factor Sp3 isoform 1) c4(135317, 135318) c5(135319, 135320, 135321, 135322, 135323, 135324, 135325, 135326, 135327, 135328, 135329, 135330, 135331, 135332, 135333, 135334, 135335, 135336, 135337, 135338, 135339, 135340, 135341, 135342, 135343) c6( ) c7(bv, gJ, hW, aw, at, gl, dl, dy, eA, V, B); #c1(SP4) c2(NP_003103) c3(transcription factor Sp4) c4(135344) c5(135345, 135346, 135347, 135348, 135349, 135350, 135351, 135352) c6( ) c7(T, hd, ac, nB, bE, WY, cz, bB, gl, dL, bK, he, Cb, X, clu); #c1(SP4) c2(XP_011513790) c3(transcription factor Sp4 isoform X5) c4(306256) c5( ) c6( ) c7(T, hd, ac, nB, bE, WY, cz, bB, gl, dL, bk, he, Cb, X, clu); #c1(SP4) c2(XP_011513791) c3(transcription factor Sp4 isoform X6) c4( ) c5(306257, 306258, 306259, 306260, 306261, 306262) c6( ) c7(T, hd, ac, nB, bE, WY, cz, bB, gl, dL, bk, he, Cb, X, clu); #c1(SP5) c2(NP_001003845) c3(transcription factor Sp5) c4(20234) c5(20235, 20236, 20237, 20238, 20239, 20240) c6( ) c7(ad); #c1(SP6) c2(NP_954871) c3(transcription factor Sp6) c4(261474) c5(261475, 261476, 261477, 261478, 261479, 261480, 261481, 261482, 261483, 261484) c6( ) c7(bL, dq, sS); #c1(SP7) c2(NP_690599) c3(transcription factor Sp7 isoform a) c4(244896, 244897) c5(244898, 244899, 244900, 244901, 244902, 244903, 244904, 244905, 244906, 244907, 244908, 244909) c6( ) c7(D, zw, buz, zt, eH, eF); #c1(SP7) c2(XP_011536203) c3(transcription factor Sp7 isoform X1) c4( ) c5(333493, 333494, 333495, 333496, 333497) c6( ) c7(D, zw, buz, zt, eH, eF); #c1(SP8) c2(NP_874359) c3(transcription factor Sp8 isoform 1) c4(256612, 256613, 256614) c5(256615, 256616, 256617, 256618, 256619, 256620, 256621) c6( ) c7(gl, T, bK); #c1(SP8) c2(XP_011513478) c3(transcription factor Sp8 isoform X1) c4( ) c5(305766, 305767, 305768, 305769, 305770, 305771, 305772, 305773, 305774, 305775, 305776) c6( ) c7(gl, T, bK); #c1(SPA17) c2(XP_011541172) c3(sperm surface protein Sp17 isoform X1) c4(339768) c5(339769, 339770, 339771, 339772, 339773, 339774, 339775, 339776, 339777, 339778) c6( ) c7(T, eM, if, ff, ht, hL, fd, m, hu, bu, en, t, qN, g, y, cl); #c1(SPACA1) c2(NP_112222) c3(sperm acrosome membrane-associated protein 1 precursor) c4(220950) c5(220951, 220952, 220953, 220954, 220955, 220956) c6( ) c7(FE); #c1(SPACA3) c2(NP_776246) c3(sperm acrosome membrane-associated protein 3) c4(251023, 251024, 251025) c5(251026, 251027, 251028, 251029, 251030, 251031, 251032, 251033, 251034, 251035, 251036, 251037) c6( ) c7(T, bu); #c1(SPAG1) c2(NP_757367) c3(sperm-associated antigen 1) c4(248736) c5(248737, 248738, 248739, 248740, 248741, 248742, 248743, 248744, 248745, 248746) c6( ) c7(T, bVm, aE, Pf, clv, eJ, MG); #c1(SPAG11A) c2(NP_001075021) c3(sperm-associated antigen 11A precursor) c4(37176) c5(37177, 37178, 37179, 37180, 37181, 37182, 37183, 37184) c6(37185, 37186) c7(ae, T, KT, bM, y, c, pf, wB, aA, aF, j, t, bau, af, ad, El); #c1(SPAG11A) c2(XP_005272448) c3(sperm-associated antigen 11A isoform X1) c4(284029, 284030, 284031) c5(284032) c6( ) c7(ae, T, kT, bM, y, c, pf, wB, aA, aF, j, t, bau, af, ad, El); #c1(SPAG11A) c2(XP_005272449) c3(sperm-associated antigen 11A isoform X2) c4(284033, 284034, 284035, 284036, 284037) c5(284038) c6( ) c7(ae, T, kT, bM, y, c, pf, wB, aA, aF, j, t, bau, af, ad, El); #c1(SPAG11A) c2(XP_005272450) c3(sperm-associated antigen 11A isoform X3) c4(284039, 284040, 284041) c5(284042, 284043, 284044, 284045, 284046, 284047, 284048, 284049, 284050) c6(284051, 284052) c7(ae, T, KT, bM, y, c, pf, wB, aA, aF, j, t, bau, af, ad, El); #c1(SPAG11B) c2(NP_057596) c3(sperm-associated antigen 11B isoform A preproprotein) c4(191537) c5(191538, 191539, 191540, 191541, 191542, 191543, 191544) c6(191545, 191546) c7(ae, T, KT, bM, y, c, pf, wB, aA, aF, j, t, bau, af, ad, El); #c1(SPAG11B) c2(NP_478107) c3(sperm-associated antigen 11B isoform G preproprotein) c4(230901) c5(230902, 230903, 230904, 230905, 230906, 230907, 230908, 230909) c6(230910, 230911) c7(ae, T, kT, bM, y, c, pf, wB, aA, aF, j, t, bau, af, ad, El); #c1(SPAG11B) c2(NP_478108) c3(sperm-associated antigen 11B isoform D preproprotein) c4(230912, 230913, 230914, 230915, 230916, 230917) c5(230918, 230919, 230920, 230921, 230922, 230923, 230924) c6(230925, 230926) c7(ae, T, kT, bM, y, c, pf, wB, aA, aF, j, t, bau, af, ad, El); #c1(SPAG11B) c2(NP_478109) c3(sperm-associated antigen 11B isoform H preproprotein) c4(230927) c5(230928, 230929, 230930, 230931, 230932, 230933, 230934) c6(230935) c7(ae, T, KT, bM, y, c, pf, wB, aA, aF, j, t, bau, af, ad, El); #c1(SPAG11B) c2(NP_478110) c3(sperm-associated antigen 11B isoform C preproprotein) c4(230936) c5(230937, 230938, 230939, 230940, 230941, 230942, 230943) c6(230944, 230945) c7(ae, T, kT, bM, y, c, pf, wB, aA, aF, j, t, bau, af, ad, El); #c1(SPAG11B) c2(NP_478113) c3(sperm-associated antigen 11B isoform B precursor) c4(230946, 230947) c5(230948, 230949, 230950, 230951, 230952, 230953) c6(230954) c7(ae, T, KT, bM, y, c, pf, wB, aA, aF, j, t, bau, af, ad, El); #c1(SPAG11B) c2(NP_478114) c3(sperm-associated antigen 11B isoform E precursor) c4(230955) c5(230956, 230957, 230958, 230959, 230960, 230961, 230962, 230963) c6(230964) c7(ae, T, kT, bM, y, c, pf, wB, aA, aF, j, t, bau, af, ad, El); #c1(SPAG16) c2(NP_001020607) c3(sperm-associated antigen 16 protein isoform 2) c4(28969) c5(28970, 28971, 28972, 28973, 28974, 28975) c6( ) c7(ac, aw, aFE, EQ, at, sj); #c1(SPAG16) c2(NP_078808) c3(sperm-associated antigen 16 protein isoform 1) c4(215567) c5(215568, 215569, 215570, 215571, 215572, 215573) c6( ) c7(ac, aw, aFE, EQ, at, sj); #c1(SPAG16) c2(XP_011510116) c3(sperm-associated antigen 16 protein isoform X1) c4(300712) c5(300713, 300714, 300715, 300716, 300717, 300718) c6( ) c7(ac, aw, aFE, EQ, at, sj); #c1(SPAG16) c2(XP_011510122) c3(sperm-associated antigen 16 protein isoform X7) c4(300719) c5(300720, 300721, 300722, 300723, 300724, 300725) c6( ) c7(ac, aw, aFE, EQ, at, sj); #c1(SPAG16) c2(XP_011510123) c3(sperm-associated antigen 16 protein isoform X8) c4(300726) c5(300727, 300728, 300729, 300730, 300731, 300732) c6( ) c7(ac, aw, aFE, EQ, at, sj); #c1(SPAG16) c2(XP_011510129) c3(sperm-associated antigen 16 protein isoform X13) c4(300733) c5(300734, 300735, 300736, 300737, 300738, 300739) c6( ) c7(ac, aw, aFE, EQ, at, sj); #c1(SPAG4) c2(NP_003107) c3(sperm-associated antigen 4 protein) c4(135376) c5(135377, 135378, 135379, 135380, 135381, 135382) c6( ) c7(es, T, fr, py); #c1(SPAG5) c2(NP_006452) c3(sperm-associated antigen 5) c4(165333, 165334) c5(165335, 165336, 165337, 165338, 165339, 165340, 165341, 165342, 165343, 165344, 165345, 165346) c6( ) c7(if, kP, qN, aT); #c1(SPAG6) c2(NP_001240783) c3(sperm-associated antigen 6 isoform 3) c4(88808) c5(88809, 88810, 88811, 88812, 88813, 88814, 88815, 88816, 88817, 88818, 88819, 88820) c6( ) c7(aW, aw, dV, aT); #c1(SPAG6) c2(NP_036575) c3(sperm-associated antigen 6 isoform 1) c4(174456, 174457, 174458) c5(174459, 174460, 174461, 174462, 174463, 174464, 174465, 174466, 174467, 174468, 174469, 174470) c6( ) c7(aW, aw, dV, aT); #c1(SPAG6) c2(NP_758442) c3(sperm-associated antigen 6 isoform 2) c4(249167, 249168) c5(249169, 249170, 249171, 249172, 249173, 249174) c6( ) c7(aW, aw, dV, aT); #c1(SPAG6) c2(XP_005252702) c3(sperm-associated antigen 6 isoform X1) c4(272019) c5( ) c6( ) c7(aW, aw, dV, aT); #c1(SPAG6) c2(XP_005252703) c3(sperm-associated antigen 6 isoform X2) c4( ) c5(272020, 272021, 272022, 272023, 272024, 272025, 272026, 272027, 272028, 272029, 272030, 272031) c6( ) c7(aW, aw, dV, aT); #c1(SPAG7) c2(NP_004881) c3(sperm-associated antigen 7) c4(150523) c5(150524, 150525, 150526, 150527, 150528, 150529) c6( ) c7(NY); #c1(SPAG8) c2(NP_001034681) c3(sperm-associated antigen 8 isoform 1) c4(32083, 32084) c5( ) c6( ) c7(bb, c, om, dq, v, K, GK); #c1(SPAG8) c2(NP_758516) c3(sperm-associated antigen 8 isoform 2) c4(249239) c5(249240, 249241, 249242, 249243, 249244, 249245) c6( ) c7(bb, c, om, dq, v, K, GK); #c1(SPAG8) c2(XP_005251493) c3(sperm-associated antigen 8 isoform X1) c4( ) c5(270949, 270950, 270951, 270952, 270953, 270954, 270955, 270956, 270957, 270958, 270959, 270960) c6( ) c7(bb, c, om, dq, v, K, GK); #c1(SPAG8) c2(XP_011516146) c3(sperm-associated antigen 8 isoform X4) c4( ) c5(309369, 309370, 309371, 309372, 309373) c6( ) c7(bb, c, om, dq, v, K, GK); #c1(SPAG9) c2(NP_001123999) c3(C-Jun-amino-terminal kinase-interacting protein 4 isoform 2) c4(48228, 48229) c5(48230, 48231, 48232, 48233, 48234, 48235, 48236, 48237, 48238, 48239, 48240, 48241) c6( ) c7(T, id, AS, if, bB, fr, Ea, qN, q, ad, XX, Vp, hd, aT, dL, AN, ae, eL, iU, aW, ff, r, en, bu, as, bz, pb, es, kP, eM, nB, bE, iA, m, B); #c1(SPAG9) c2(NP_001124000) c3(C-Jun-amino-terminal kinase-interacting protein 4 isoform 1) c4(48242, 48243, 48244) c5(48245, 48246, 48247, 48248, 48249, 48250, 48251, 48252, 48253, 48254, 48255, 48256, 48257, 48258, 48259, 48260, 48261, 48262, 48263, 48264, 48265, 48266, 48267, 48268) c6( ) c7(T, id, AS, if, bB, fr, Ea, qN, q, ad, XX, Vp, hd, aT, dL, AN, ae, eL, iU, aW, ff, r, en, bu, as, bz, pb, es, kP, eM, nB, bE, iA, m, B); #c1(SPAG9) c2(NP_001238900) c3(C-Jun-amino-terminal kinase-interacting protein 4 isoform 4) c4(88040) c5(88041, 88042, 88043, 88044, 88045, 88046) c6( ) c7(T, id, AS, if, bB, fr, Ea, qN, q, ad, XX, Vp, hd, aT, dL, AN, ae, eL, iU, aW, ff, r, en, bu, as, bz, pb, es, kP, eM, nB, bE, iA, m, B); #c1(SPAG9) c2(NP_003962) c3(C-Jun-amino-terminal kinase-interacting protein 4 isoform 3) c4(142486) c5(142487, 142488, 142489, 142490, 142491, 142492) c6( ) c7(T, id, AS, if, bB, fr, Ea, qN, q, ad, XX, Vp, hd, aT, dL, AN, ae, eL, iU, aW, ff, r, en, bu, as, bz, pb, es, kP, eM, nB, bE, iA, m, B); #c1(SPAG9) c2(XP_005257825) c3(C-Jun-amino-terminal kinase-interacting protein 4 isoform X2) c4(275371) c5( ) c6( ) c7(T, id, AS, if, bB, fr, Ea, qN, q, ad, XX, Vp, hd, aT, dL, AN, ae, eL, iU, aW, ff, r, en, bu, as, bz, pb, es, kP, eM, nB, bE, iA, m, B); #c1(SPAG9) c2(XP_005257826) c3(C-Jun-amino-terminal kinase-interacting protein 4 isoform X3) c4(275372) c5( ) c6( ) c7(T, id, AS, if, bB, fr, Ea, qN, q, ad, XX, Vp, hd, aT, dL, AN, ae, eL, iU, aW, ff, r, en, bu, as, bz, pb, es, kP, eM, nB, bE, iA, m, B); #c1(SPAG9) c2(XP_005257831) c3(C-Jun-amino-terminal kinase-interacting protein 4 isoform X9) c4( ) c5(275373, 275374, 275375, 275376, 275377, 275378) c6( ) c7(T, id, AS, if, bB, fr, Ea, qN, q, ad, XX, Vp, hd, aT, dL, AN, ae, eL, iU, aW, ff, r, en, bu, as, bz, pb, es, kP, eM, nB, bE, iA, m, B); #c1(SPAM1) c2(XP_011514825) c3 (hyaluronidase PH-20 isoform X1) c4(307511) c5(307512, 307513, 307514, 307515, 307516, 307517, 307518, 307519) c6(307520, 307521) c7(T, ft, aW, eL, go, ff, hL, aT, n, dL, ci, m, en, t, ik, ae, ad, cl); #c1(SPAM1) c2(XP_011514826) c3(hyaluronidase PH-20 isoform X2) c4(307522, 307523) c5( ) c6( ) c7(T, ft, aW, eL, go, ff, hL, aT, n, dl, ci, m, en, t, ik, ae, ad, cl); #c1(SPAM1) c2(XP_011514827) c3 (hyaluronidase PH-20 isoform X3) c4( ) c5(307524, 307525, 307526, 307527, 307528, 307529, 307530, 307531, 307532, 307533, 307534, 307535, 307536, 307537, 307538, 307539, 307540, 307541, 307542, 307543, 307544, 307545, 307546, 307547, 307548, 307549) c6(307550, 307551, 307552, 307553, 307554, 307555, 307556, 307557) c7(T, ft, aW, eL, go, ff, hL, aT, n, dL, ci, m, en, t, ik, ae, ad, cl); #c1(SPAM1) c2(XP_011514828) c3(hyaluronidase PH-20 isoform X4) c4( ) c5(307558, 307559, 307560, 307561, 307562, 307563, 307564) c6(307565) c7(T, ft, aW, eL, go, ff, hL, aT, n, dL, ci, m, en, t, ik, ae, ad, cl); #c1(SPANXA2) c2(NP_663695) c3(sperm protein associated with the nucleus on the X chromosome A) c4(241421, 241422) c5(241423, 241424, 241425, 241426, 241427, 241428, 241429, 241430, 241431, 241432, 241433, 241434, 241435, 241436, 241437) c6( ) c7(Bl, T, gU, ND, vT, wu, il, Y, eD, r, cj, vH, fd, wm, bu, t, bt, ae, q, ad); #c1(SPANXB1) c2(NP_115850) c3(sperm protein associated with the nucleus on the X chromosome B/F) c4(224259) c5(224260, 224261, 224262, 224263, 224264, 224265) c6( ) c7(gU, ND, Y, vH, ae, ad); #c1(SPANXD) c2(NP_115793) c3(sperm protein associated with the nucleus on the X chromosome D) c4(223918) c5(223919, 223920, 223921, 223922, 223923, 223924, 223925, 223926, 223927, 223928) c6( ) c7(vH, ae, gU, ad, ND); #c1(SPANXN4) c2(NP_001009613) c3(sperm protein associated with the nucleus on the X chromosome N4) c4(23680) c5(23681, 23682, 23683, 23684, 23685, 23686) c6( ) c7(oe, fq); #c1(SPARC) c2(NP_003109) c3(SPARC precursor) c4(135383, 135384) c5(135385, 135386, 135387, 135388, 135389, 135390, 135391, 135392, 135393, 135394, 135395, 135396, 135397, 135398, 135399, 135400, 135401, 135402) c6( ) c7(T, aC, Js, pf, ea, if, al, fd, bB, lR, ci, cab, ip, xE, aE, ari, iv, Ou, K, apj, eL, cp, xQ, dH, Jl, aW, hl, g, as, eF, ir, uC, cm, bw, eM, iA, vy, m, cE, Vj, dD, zX, zt, aw, n, aj, fr, qN, zw, alu, dL, pj, uc, clw, dx, mR, MD, Xg, fQ, kp, dw, dB, anC, uw, mg, hL, pg, sa, it, B, Jo, bL, dA, cHB, dk, fG, aA, cz, wo, v, j, AO, q, J, eA, yg, aT, dz, zp, ae, af, bA, aAi, jf, iF, il, Y, la, r, lo, cl, t, Vq, pb, ov, vK, acW, pZ, DW, gX, dy, bt, sY, aZ, eq, ad, qC, bRP, av, jo, uX, d, lR, et, eH, aF, bz, LB, es, D, bE, aFb, y, aUg, X); #c1(SPARCL1) c2(NP_001121782) c3(SPARC-like protein 1 isoform 1 precursor) c4(46151, 46152, 46153, 46154, 46155, 46156, 46157, 46158) c5(46159, 46160, 46161, 46162, 46163, 46164, 46165, 46166, 46167, 46168, 46169, 46170, 46171, 46172, 46173, 46174, 46175, 46176, 46177, 46178, 46179, 46180, 46181, 46182, 46183, 46184, 46185, 46186, 46187, 46188, 46189, 46190, 46191, 46192, 46193, 46194, 46195, 46196, 46197, 46198, 46199, 46200, 46201, 46202, 46203, 46204, 46205, 46206, 46207, 46208, 46209, 46210, 46211, 46212, 46213, 46214, 46215, 46216, 46217, 46218, 46219, 46220, 46221, 46222, 46223, 46224, 46225, 46226, 46227, 46228, 46229, 46230, 46231, 46232) c6(46233, 46234, 46235, 46236, 46237, 46238, 46239, 46240, 46241, 46242, 46243, 46244, 46245, 46246, 46247, 46248, 46249, 46250) c7(T, aC, ha, aA, bB, ci, V, eA, ad, hX, dP, av, aE, ae, r, kp, aF, t, eM, bE, m, B); #c1(SPAST) c2(NP_055761) c3(spastin isoform 1) c4(183329) c5(183330, 183331, 183332, 183333, 183334, 183335) c6( ) c7(cjZ, aiZ, aiY, Ol, amp, ha, cly, rf, aeu, uu, nA, bX, il, V, aR, Ks, LN, no, aV, lu, hb, af, axW, bMm, LO, ajb, TQ, gl, bQ, dr, Qn, clx, ky, c, bhD, lq, U, cB); #c1(SPAST) c2(NP_955468) c3(spastin isoform 2) c4(261807) c5(261808, 261809, 261810, 261811, 261812, 261813) c6( ) c7(cjZ, aiZ, aiY, Ol, amp, ha, cly, rf, aeu, uu, nA, bX, il, V, aR, Ks, LN, no, aV, lu, hb, af, axW, bMm, LO, ajb, TQ, gl, bQ, dr, Qn, clx, ky, c, bhD, lq, U, cB); #c1(SPAST) c2(XP_011531369) c3(spastin isoform X2) c4( ) c5(327375) c6( ) c7(cjZ, aiZ, aiY, Ol, amp, ha, cly, rf, aeu, uu, nA, bX, il, V, aR, Ks, LN, no, aV, lu, hb, af, axW, bMm, LO, ajb, TQ, gl, bQ, dr, Qn, clx, ky, c, bhD, lq, U, cB); #c1(SPATA13) c2(NP_001159743) c3(spermatogenesis-associated protein 13 isoform 1) c4(64663, 64664) c5( ) c6( ) c7(bF, eu, ac, s); #c1(SPATA13) c2(NP_001273721) c3(spermatogenesis-associated protein 13 isoform 3) c4(105709, 105710) c5(105711, 105712, 105713, 105714, 105715, 105716, 105717, 105718, 105719, 105720, 105721, 105722, 105723) c6( ) c7(bF, eu, ac, s); #c1(SPATA13) c2(NP_001273723) c3(spermatogenesis-associated protein 13 isoform 5) c4(105724) c5(105725, 105726, 105727, 105728, 105729, 105730) c6( ) c7(bF, eu, ac, s); #c1(SPATA13) c2(NP_694568) c3(spermatogenesis-associated protein 13 isoform 2) c4( ) c5(245291, 245292, 245293, 245294, 245295, 245296, 245297, 245298, 245299, 245300, 245301) c6( ) c7(bF, eu, ac, s); #c1(SPATA16) c2(NP_114161) c3(spermatogenesis-associated protein 16) c4(222468) c5(222469, 222470, 222471, 222472, 222473, 222474) c6( ) c7(bEw, gU, ad); #c1(SPATA17) c2(XP_005273109) c3(spermatogenesis-associated protein 17 isoform X1) c4(284298) c5(284299, 284300, 284301, 284302, 284303, 284304) c6( ) c7(vH, iX, gU); #c1(SPATA18) c2(NP_660306) c3(mitochondria-eating protein isoform 1) c4(240853) c5(240854, 240855, 240856, 240857, 240858, 240859) c6( ) c7(km); #c1(SPATA18) c2(XP_005265775) c3(mitochondria-eating protein isoform X6) c4( ) c5(279717) c6( ) c7(km); #c1(SPATA18) c2(XP_011532671) c3(mitochondria-eating protein isoform X1) c4( ) c5(328721) c6( ) c7(km); #c1(SPATA19) c2(NP_001278921) c3(spermatogenesis-associated protein 19, mitochondrial precursor) c4(110998) c5(110999, 111000, 111001, 111002, 111003, 111004, 111005, 111006, 111007, 111008) c6( ) c7(ae, T, sS, ad); #c1(SPATA2) c2(NP_001129245) c3(spermatogenesis-associated protein 2) c4(51319, 51320) c5(51321, 51322, 51323, 51324, 51325, 51326, 51327, 51328, 51329, 51330, 51331, 51332, 51333, 51334, 51335, 51336) c6( ) c7(T, A, iM, qZ, je, aW, Y, jx, cr, al, uo, kH, lo, h, eZ, y, hv); #c1(SPATA20) c2(NP_001245301) c3(spermatogenesis-associated protein 20 isoform 2 precursor) c4(93035, 93036, 93037, 93038) c5(93039, 93040, 93041, 93042, 93043, 93044) c6( ) c7(jz); #c1(SPATA20) c2(NP_001245302) c3(spermatogenesis-associated protein 20 isoform 3) c4( ) c5(93045, 93046, 93047, 93048, 93049) c6( ) c7(jz); #c1(SPATA20) c2(NP_073738) c3(spermatogenesis-associated protein 20 isoform 1 precursor) c4(213343) c5(213344, 213345, 213346, 213347, 213348, 213349, 213350, 213351, 213352, 213353, 213354, 213355, 213356, 213357, 213358, 213359) c6( ) c7(jz); #c1(SPATA21) c2(XP_011539714) c3(spermatogenesis-associated protein 21 isoform X1) c4( ) c5(337939) c6( ) c7(aq, kl); #c1(SPATA21) c2(XP_011539716) c3(spermatogenesis-associated protein 21 isoform X3) c4(337940) c5( ) c6( ) c7(aq, kl); #c1(SPATA21) c2(XP_011539719) c3(spermatogenesis-associated protein 21 isoform X6) c4( ) c5(337941, 337942, 337943, 337944, 337945, 337946) c6( ) c7(aq, kl); #c1(SPATA21) c2(XP_011539722) c3(spermatogenesis-associated protein 21 isoform X8) c4(337947) c5(337948, 337949, 337950, 337951, 337952, 337953, 337954) c6( ) c7(aq, kl); #c1(SPATA22) c2(NP_001164166) c3(spermatogenesis-associated protein 22 isoform 2) c4(66686) c5(66687, 66688, 66689, 66690, 66691, 66692, 66693, 66694, 66695, 66696, 66697, 66698, 66699, 66700) c6( ) c7(vH, ND); #c1(SPATA22) c2(NP_001164167) c3(spermatogenesis-associated protein 22 isoform 3) c4(66701) c5(66702, 66703, 66704, 66705, 66706, 66707) c6( ) c7(vH, ND); #c1(SPATA22) c2(NP_001164170) c3(spermatogenesis-associated protein 22 isoform 1) c4(66708) c5(66709, 66710, 66711, 66712, 66713, 66714) c6( ) c7(vH, ND); #c1(SPATA22) c2(XP_005256887) c3(spermatogenesis-associated protein 22 isoform X4) c4(274558) c5(274559, 274560, 274561, 274562, 274563, 274564) c6( ) c7(vH, ND); #c1(SPATA25) c2(NP_542175) c3(spermatogenesis-associated protein 25) c4(232184) c5(232185, 232186, 232187, 232188, 232189, 232190) c6( ) c7(vH); #c1(SPATA25) c2(XP_011526833) c3(spermatogenesis-associated protein 25 isoform X1) c4( ) c5(321191) c6( ) c7(vH); #c1(SPATA5) c2(NP_660208) c3(spermatogenesis-associated protein 5) c4(240552) c5(240553, 240554, 240555, 240556, 240557, 240558) c6( ) c7(OW); #c1(SPATA7) c2(NP_001035518) c3(spermatogenesis-associated protein 7 isoform 2) c4(32842) c5(32843, 32844, 32845, 32846, 32847, 32848, 32849, 32850, 32851, 32852) c6( ) c7(alF, nx, ci, nC, ae, cyP, ad); #c1(SPATA7) c2(NP_060888) c3(spermatogenesis-associated protein 7 isoform 1) c4(198409, 198410) c5(198411, 198412, 198413, 198414, 198415, 198416) c6( ) c7(alF, nx, ci, nC, ae, cyP, ad); #c1(SPATA7) c2(XP_006720268) c3(spermatogenesis-associated protein 7 isoform X8) c4( ) c5(293335, 293336, 293337, 293338, 293339, 293340, 293341, 293342) c6( ) c7(alF, nx, ci, nC, ae, cyP, ad); #c1(SPATA8) c2(NP_775770) c3(spermatogenesis-associated protein 8 isoform 1) c4(250243) c5(250244, 250245, 250246, 250247, 250248, 250249) c6( ) c7(aw, cd, ac, an); #c1(SPATA9) c2(NP_114158) c3(spermatogenesis-associated protein 9) c4(222423, 222424) c5(222425, 222426, 222427, 222428, 222429, 222430, 222431, 222432, 222433, 222434, 222435, 222436, 222437, 222438, 222439, 222440) c6( ) c7(nJ, gU, Ul); #c1(SPATC1) c2(NP_001127846) c3(speriolin isoform 2) c4(49367) c5(49368, 49369, 49370, 49371, 49372, 49373) c6( ) c7(dt, jX); #c1(SPATC1) c2(NP_940974) c3(speriolin isoform 1) c4(260223) c5(260224, 260225, 260226, 260227, 260228, 260229) c6( ) c7(dt, jX); #c1(SPC24) c2(NP_872319) c3(kinetochore protein Spc24) c4(256114) c5(256115, 256116, 256117, 256118, 256119, 256120, 256121, 256122, 256123, 256124) c6( ) c7(aw); #c1(SPC24) c2(XP_005259810) c3 (kinetochore protein Spc24 isoform X2) c4(276306) c5(276307, 276308, 276309, 276310, 276311, 276312) c6( ) c7(aw); #c1(SPC25) c2(NP_065726) c3(kinetochore protein Spc25) c4(205070) c5(205071, 205072, 205073, 205074, 205075, 205076) c6( ) c7(ey); #c1(SPC25) c2(XP_011545240) c3(kinetochore protein Spc25 isoform X1) c4(344052) c5( ) c6( ) c7(ey); #c1(SPC25) c2(XP_011545242) c3 (kinetochore protein Spc25 isoform X3) c4( ) c5(344053, 344054, 344055, 344056, 344057, 344058) c6( ) c7(ey); #c1(SPCS3) c2(NP_068747) c3(signal peptidase complex subunit 3) c4(210163) c5(210164, 210165, 210166, 210167, 210168, 210169) c6( ) c7(cd, j); #c1(SPDEF) c2(NP_001239223) c3(SAM pointed domain-containing Ets transcription factor isoform 2) c4(88377) c5(88378, 88379, 88380, 88381, 88382) c6( ) c7(ae, T, s, bE, aW, ayS, X, aT, r, adR, bdp, bB, m, ct, ci, g, t, bz, q, ad); #c1(SPDEF) c2(NP_036523) c3(SAM pointed domain-containing Ets transcription factor isoform 1) c4( ) c5(174227, 174228, 174229, 174230, (74231) c6( ) c7(ae, T, s, bE, aW, ayS, X, aT, r, adR, bdp, bB, m, ct, ci, g, t, bz, q, ad); #c1(SPDEF) c2(XP_005249045) c3(SAM pointed domain-containing Ets transcription factor isoform X1) c4(268951) c5(268952, 268953, 268954) c6( ) c7(ae, T, s, bE, aW, ayS, X, aT, r, adR, bdp, bB, m, ct, ci, g, t, bz, q, ad); #c1(SPDL1) c2(NP_060255) c3(protein Spindly) c4(195048, 195049, 195050, 195051, 195052) c5(195053, 195054, 195055, 195056, 195057, 195058, 195059, 195060, 195061, 195062, 195063, 195064, 195065, 195066, 195067, 195068, 195069, 195070, 195071, 195072, 195073, 195074, 195075, 195076, 195077, 195078, 195079, 195080, 195081, 195082, 195083, 195084, 195085, 195086, 195087, 195088, 195089, 195090, 195091, 195092, 195093, 195094) c6( ) c7(bX); #c1(SPDL1) c2(XP_011532891) c3(protein Spindly isoform X1) c4(329071, 329072) c5( ) c6( ) c7(bX); #c1(SPDYA) c2(NP_001008779) c3(speedy protein A isoform 2) c4( ) c5(23397, 23398, 23399, 23400, 23401, 23402) c6( ) c7(T, aW, aT, n, eA, hE, B); #c1(SPDYA) c2(NP_001136106) c3(speedy protein A isoform 1) c4(54355, 54356) c5(54357, 54358, 54359, 54360, 54361, 54362, 54363, 54364, 54365, 54366) c6( ) c7(T, aW, aT, n, eA, hE, B); #c1(SPECC1) c2(NP_001028726) c3(cytospin-B isoform 4) c4(30519, 30520) c5(30521, 30522, 30523, 30524, 30525, 30526, 30527) c6( ) c7(aW, ds, tT, anV, aaG, wc, aT, pR, act, pq, q, aa); #c1(SPECC1) c2(NP_001028727) c3(cytospin-B isoform 2) c4(30528) c5(30529, 30530, 30531, 30532, 30533, 30534) c6( ) c7(aW, ds, tT, anV, aaG, wc, aT, pR, act, pq, q, aa); #c1(SPECC1) c2(NP_001230367) c3(cytospin-B isoform 5) c4( ) c5(86256, 86257, 86258, 86259, 86260) c6( ) c7(aW, ds, tT, anV, aaG, wc, aT, pR, act, pq, q, aa); #c1(SPECC1) c2(NP_001230368) c3(cytospin-B isoform 1) c4(86261) c5(86262, 86263, 86264, 86265, 86266, 86267) c6( ) c7(aW, ds, tT, anV, aaG, wc, al, pR, act, pq, q, aa); #c1(SPECC1) c2(NP_690868) c3(cytospin-B isoform 3) c4(245026, 245027) c5(245028, 245029, 245030, 245031, 245032, 245033) c6( ) c7(aW, ds, tT, anV, aaG, wc, aT, pR, act, pq, q, aa); #c1(SPECC1) c2(XP_005256917) c3(cytospin-B isoform X4) c4( ) c5(274577, 274578, 274579, 274580, 274581) c6( ) c7(aW, ds, tT, anV, aaG, wc, al, pR, act, pq, q, aa); #c1(SPECC1) c2(XP_011522375) c3(cytospin-B isoform X1) c4(317048) c5(317049, 317050, 317051, 317052, 317053, 317054) c6( ) c7(aW, ds, tT, anV, aaG, wc, aT, pR, act, pq, q, aa); #c1(SPECC1) c2(XP_011522376) c3(cytospin-B isoform X2) c4(317055) c5(317056) c6( ) c7(aW, ds, tT, anV, aaG, wc, aT, pR, act, pq, q, aa); #c1(SPECC1) c2(XP_011522378) c3(cytospin-B isoform X3) c4( ) c5(317057, 317058, 317059, 317060, 317061, 317062) c6( ) c7(aW, ds, tT, anV, aaG, wc, al, pR, act, pq, q, aa); #c1(SPECC1L) c2(NP_001138940) c3(cytospin-A isoform 1) c4(58436) c5(58437, 58438, 58439, 58440, 58441, 58442, 58443, 58444, 58445, 58446) c6( ) c7(azs, clz); #c1(SPECC1L) c2(NP_001241661) c3(cytospin-A isoform 2) c4(89000, 89001, 89002) c5(89003, 89004, 89005, 89006, 89007, 89008, 89009, 89010, 89011, 89012, 89013, 89014, 89015, 89016, 89017, 89018, 89019, 89020) c6( ) c7(azs, clz); #c1(SPEF2) c2(NP_079143) c3(sperm flagellar protein 2 isoform 1) c4(217229) c5(217230, 217231, 217232, 217233, 217234, 217235) c6( ) c7(V); #c1(SPEF2) c2(NP_653323) c3(sperm flagellar protein 2 isoform 2) c4(239595) c5(239596, 239597, 239598, 239599, 239600, 239601) c6( ) c7(V); #c1(SPEF2) c2(XP_011512438) c3(sperm flagellar protein 2 isoform X2) c4(303128) c5(303129, 303130, 303131, 303132, 303133, 303134) c6( ) c7(V); #c1(SPEG) c2(NP_001166947) c3(striated muscle preferentially expressed protein kinase isoform 4) c4(68700) c5(68701, 68702, 68703, 68704, 68705, 68706, 68707, 68708, 68709, 68710, 68711) c6( ) c7(ea, xz, mx, UX); #c1(SPEG) c2(NP_005867) c3(striated muscle preferentially expressed protein kinase isoform 1) c4(160004, 160005) c5(160006, 160007, 160008, 160009, 160010, 160011, 160012, 160013, 160014, 160015, 160016, 160017) c6( ) c7(ea, xz, mx, UX); #c1(SPEG) c2(XP_005246296) c3(striated muscle preferentially expressed protein kinase isoform X9) c4(267028) c5( ) c6( ) c7(ea, xz, mx, UX); #c1(SPEG) c2(XP_011508781) c3(striated muscle preferentially expressed protein kinase isoform X1) c4( ) c5(299376, 299377) c6( ) c7(ea, xz, mx, UX); #c1(SPEG) c2(XP_011508783) c3(striated muscle preferentially expressed protein kinase isoform X3) c4( ) c5(299378, 299379) c6( ) c7(ea, xz, mx, UX); #c1(SPEG) c2(XP_011508784) c3(striated muscle preferentially expressed protein kinase isoform X4) c4( ) c5(299380) c6( ) c7(ea, xz, mx, UX); #c1(SPEG) c2(XP_011508788) c3(striated muscle preferentially expressed protein kinase isoform X13) c4( ) c5(299381, 299382, 299383, 299384, 299385, 299386, 299387) c6( ) c7(ea, xz, mx, UX); #c1(SPESP1) c2(NP_663633) c3(sperm equatorial segment protein 1 precursor) c4(241389) c5(241390, 241391, 241392, 241393, 241394, 241395) c6( ) c7(bL, bt, dy, eR); #c1(SPG11) c2(NP_001153699) c3(spatacsin isoform 2) c4(60883) c5(60884, 60885, 60886, 60887, 60888, 60889) c6( ) c7(bcR, clC, bAn, c, bcS, clA, lu, aV, dM, clB, Kk, TO, ha, U, nw, bk, nA, Qn); #c1(SPG11) c2(NP_079413) c3(spatacsin isoform 1) c4(218021, 218022) c5(218023, 218024, 218025, 218026, 218027, 218028, 218029, 218030, 218031, 218032, 218033, 218034) c6( ) c7(bcR, clC, bAn, c, bcS, clA, lu, aV, dM, clB, Kk, TO, ha, U, nw, bk, nA, Qn); #c1(SPG20) c2(NP_055902) c3(spartin) c4(184340) c5(184341, 184342, 184343, 184344, 184345, 184346, 184347, 184348, 184349, 184350, 184351, 184352, 184353, 184354, 184355, 184356, 184357, 184358) c6( ) c7(T, s, bcS, aV, pf, r, bl, bkk, q); #c1(SPG21) c2(NP_001121362) c3(maspardin isoform b) c4(45759) c5(45760, 45761, 45762, 45763, 45764, 45765) c6( ) c7(ok, Qn, Xd, aV, clD, ao); #c1(SPG21) c2(NP_057714) c3(maspardin isoform a) c4(192494) c5(192495, 192496, 192497, 192498, 192499, 192500, 192501, 192502, 192503, 192504) c6( ) c7(ok, Qn, Xd, aV, clD, ao); #c1(SPG7) c2(NP_003110) c3(paraplegin isoform 1 precursor) c4(135403, 135404) c5(135405, 135406, 135407, 135408, 135409, 135410, 135411, 135412, 135413) c6( ) c7(gV, T, dA, qb, Ol, clE, jh, fd, bZ, v, ci, yV, q, ad, eA, eJ, eW, aV, clA, aN, aT, dL, ey, awL, ae, af, eL, PP, bcS, s, aW, Y, OX, oV, hG, axW, r, lo, t, cta, kC, cm, On, ck, kT, kB, mx, U, cj, iS, MJ, awK, m, gT, bt, B); #c1(SPG7) c2(NP_955399) c3(paraplegin isoform 2 precursor) c4(261702) c5(261703, 261704, 261705, 261706, 261707, 261708, 261709) c6( ) c7(gV, T, dA, qb, Ol, clE, jh, fd, bZ, v, ci, yV, q, ad, eA, eJ, eW, aV, clA, aN, aT, dL, ey, awL, ae, af, eL, PP, bcS, s, aW, Y, OX, oV, hG, axW, r, lo, t, cta, kC, cm, On, ck, KT, KB, mx, U, cj, iS, MJ, awK, m, gT, bt, B); #c1(SPG7) c2(XP_005256378) c3(paraplegin isoform X4) c4( ) c5(274132, 274133, 274134, 274135, 274136, 274137) c6( ) c7(gV, T, dA, qb, Ol, clE, jh, fd, bZ, v, ci, yV, q, ad, eA, eJ, eW, aV, clA, aN, aT, dL, ey, awL, ae, af, eL, PP, bcS, s, aW, Y, OX, oV, hG, axW, r, lo, t, cta, kC, cm, Qn, ck, kT, kB, mx, U, cj, iS, MJ, awK, m, gT, bt, B); #c1(SPG7) c2(XP_006721327) c3(paraplegin isoform X1) c4(293948) c5(293949, 293950, 293951) c6( ) c7(gV, T, dA, qb, Ol, clE, jh, fd, bZ, v, ci, yV, q, ad, eA, eJ, eW, aV, clA, aN, aT, dl, ey, awL, ae, af, eL, PP, bcS, s, aW, Y, OX, oV, hG, axW, r, lo, t, cta, kC, cm, On, ck, KT, kB, mx, U, cj, iS, MJ, awk, m, gT, bt, B); #c1(SPHK1) c2(NP_068807) c3(sphingosine kinase 1 isoform 1) c4(210660) c5(210661, 210662, 210663, 210664, 210665) c6( ) c7(bL, T, qb, fG, ht, jN, eX, aA, cz, n, bB, dG, v, ci, C, Fb, fd, q, ad, ai, eA, mj, hd, ib, aE, aT, CO, eV, ji, K, hu, KV, ae, De, d, s, nO, iU, aW, Y, ff, eH, r, fe, dq, aq, aF, zC, en, mo, pl, t, g, as, eF, op, iR, bw, LB, ck, es, kP, eM, nB, bE, iA, WS, vA, AH, X, uR, m, Hb, qF, B, hS); #c1(SPHK1) c2(NP_892010) c3(sphingosine kinase 1 isoform 2) c4(257577, 257578, 257579) c5(257580, 257581, 257582, 257583, 257584, 257585, 257586, 257587, 257588, 257589) c6( ) c7(bL, T, qb, fG, ht, jN, eX, aA, cz, n, bB, dG, v, ci, C, Fb, fd, q, ad, ai, eA, mj, hd, ib, aE, aT, CA, eV, ji, K, hu, KV, ae, de, d, s, nO, iU, aW, Y, ff, eH, r, fe, dq, aq, aF, zC, en, mo, pl, t, g, as, eF, op, iR, bw, LB, ck, es, kP, eM, nB, bE, iA, WS, vA, AH, X, uR, m, Hb, qF, B, hS); #c1 (SPHK1) c2(XP_011523741) c3(sphingosine kinase 1 isoform X1) c4( ) c5(318219, 318220, 318221, 318222, 318223, 318224, 318225, 318226, 318227, 318228, 318229, 318230, 318231, 318232, 318233, 318234, 318235, 318236) c6(318237) c7(bL, T, qb, fG, ht, jN, eX, aA, cz, n, bB, dG, v, ci, C, Fb, fd, q, ad, ai, eA, mj, hd, ib, aE, al, CQ, eV, ji, K, hu, KV, ae, De, d, s, nO, iU, aW, Y, ff, eH, r, fe, dq, aq, aF, zC, en, mo, pl, t, g, as, eF, op, iR, bw, LB, ck, es, kP, eM, nB, bE, iA, WS, vA, AH, X, uR, m, Hb, qF, B, hS); #c1(SPHK2) c2(NP_001191087) c3(sphingosine kinase 2 isoform b) c4( ) c5(80979) c6( ) c7(ck, x, T, d, ac, eM, nO, aW, wO, fG, CM, aT, aA, aF, k, as, eA); #c1(SPHK2) c2(XP_006723354) c3(sphingosine kinase 2 isoform X1) c4(295150, 295151, 295152, 295153, 295154) c5(295155, 295156, 295157, 295158, 295159, 295160, 295161, 295162, 295163, 295164, 295165, 295166, 295167, 295168, 295169, 295170, 295171, 295172, 295173, 295174, 295175, 295176, 295177, 295178) c6( ) c7(ck, x, T, d, ac, eM, nO, aW, wO, fG, CM, aT, aA, aF, k, as, eA); #c1(SPHK2) c2(XP_011525436) c3(sphingosine kinase 2 isoform X2) c4(319856) c5(319857, 319858, 319859, 319860, 319861, 319862, 319863, 319864, 319865, 319866) c6( ) c7(ck, x, T, d, ac, eM, nO, aW, wO, fG, CM, aT, aA, aF, k, as, eA); #c1(SPHK2) c2(XP_011525437) c3(sphingosine kinase 2 isoform X3) c4( ) c5(319867, 319868, 319869, 319870, 319871) c6( ) c7(ck, x, T, d, ac, eM, nO, aW, wO, fG, CM, aT, aA, aF, k, as, eA); #c1(SPHKAP) c2(NP_001136116) c3(A-kinase anchor protein SPHKAP isoform 1) c4(54367) c5(54368, 54369, 54370, 54371, 54372, 54373) c6( ) c7(Dy, ac, Y, cm, bt, eA, Ex); #c1(SPHKAP) c2(NP_085126) c3(A-kinase anchor protein SPHKAP isoform 2) c4(219331) c5(219332, 219333, 219334, 219335, 219336, 219337) c6( ) c7(Dy, ac, Y, cm, bt, eA, Ex); #c1(SPI1) c2(NP_001074016) c3(transcription factor PU.1 isoform 1) c4(36973) c5(36974, 36975, 36976, 36977, 36978, 36979) c6( ) c7(T, fM, lk, fR, clF, eX, jh, fd, C, x, eV, ji, K, jj, iU, eZ, iV, Hv, ck, bnN, iT, cj, bt); #c1(SPI1) c2(NP_003111) c3(transcription factor PU.1 isoform 2) c4(135414) c5(135415, 135416, 135417, 135418, 135419, 135420, 135421) c6( ) c7(T, fM, lk, fR, clF, ex, jh, fd, C, x, eV, ji, K, jj, il, eZ, iV, Hv, ck, bnN, iT, cj, bt); #c1(SPIB) c2(NP_001230927) c3(transcription factor Spi-B isoform 2) c4(87191) c5(87192, 87193, 87194, 87195, 87196, 87197) c6( ) c7(ck, Of, r, Og, q, pi, bc); #c1(SPIB) c2(NP_003112) c3(transcription factor Spi-B isoform 1) c4(135422) c5(135423, 135424, 135425, 135426, 135427, 135428, 135429, 135430, 135431, 135432, 135433) c6( ) c7(ck, Of, r, Og, q, pi, bc); #c1(SPIC) c2(NP_689536) c3(transcription factor Spi-C) c4(243015) c5(243016, 243017, 243018, 243019, 243020, 243021, 243022, 243023, 243024, 243025, 243026) c6( ) c7(q, r); #c1(SPIDR) c2(NP_001073863) c3(DNA repair-scaffolding protein isoform 1) c4(36161) c5(36162, 36163, 36164, 36165, 36166, 36167) c6( ) c7(x, T, q, k, r); #c1(SPIDR) c2(NP_001269845) c3(DNA repair-scaffolding protein isoform 2) c4(102941) c5(102942, 102943, 102944, 102945, 102946, 102947) c6( ) c7(x, T, q, k, r); #c1(SPIN1) c2(NP_006708) c3(spindlin-1) c4(167319) c5(167320, 167321, 167322, 167323, 167324, 167325, 167326, 167327, 167328, 167329) c6( ) c7(t, T, ff, m, en); #c1(SPIN2A) c2(NP_061876) c3(spindlin-2A) c4(200888) c5(200889, 200890, 200891, 200892, 200893, 200894, 200895, 200896) c6( ) c7(fQ, agx, agz); #c1(SPINK1) c2(NP_003113) c3(pancreatic secretory trypsin inhibitor precursor) c4(135434) c5(135435, 135436, 135437, 135438, 135439, 135440) c6( ) c7(bL, Mb, T, aC, eR, cwb, al, jN, aA, bB, n, aj, jh, j, ci, fr, aMd, iG, aY, alx, J, q, ad, hX, eJ, ZZ, bEN, aE, oz, nX, bJj, aMj, aT, h, jH, dL, wb, aKE, aCO, aKG, dQ, ais, ae, dF, azh, eL, A, s, iU, qt, aW, oV, ff, fc, r, aq, hp, en, g, t, cff, HI, OC, es, gk, ar, GU, iA, bE, X, gX, fP, aMc, zl, bEM, m, lS, ei, aKr, bt, B, ao, ayZ, vi); #c1(SPINK2) c2(NP_001258647) c3(serine protease inhibitor Kazal-type 2 isoform 1 precursor) c4(96264) c5(96265, 96266, 96267, 96268, 96269, 96270) c6( ) c7(ck, T, hp, jh); #c1(SPINK2) c2(NP_001258649) c3(serine protease inhibitor Kazal-type 2 isoform 4 precursor) c4(96271) c5(96272, 96273, 96274, 96275, 96276, 96277) c6( ) c7(ck, T, hp, jh); #c1(SPINK2) c2(NP_066937) c3(serine protease inhibitor Kazal-type 2 isoform 2 precursor) c4(207587) c5(207588, 207589, 207590, 207591, 207592, 207593) c6( ) c7(ck, T, hp, jh); #c1(SPINK2) c2(XP_011532707) c3(serine protease inhibitor Kazal-type 2 isoform X1) c4(328741) c5( ) c6( ) c7(ck, T, hp, jh); #c1(SPINK4) c2(NP_055286) c3(serine protease inhibitor Kazal-type 4 precursor) c4(180296, 180297) c5(180298, 180299, 180300, 180301, 180302, 180303, 180304, 180305, 180306, 180307, 180308, 180309, 180310, 180311, 180312, 180313) c6( ) c7(pX, Ja, hp); #c1(SPINK5) c2(NP_001121170) c3(serine protease inhibitor Kazal-type 5 isoform a precursor) c4(45324) c5(45325, 45326, 45327, 45328, 45329, 45330, 45331, 45332, 45333, 45334, 45335, 45336) c6( ) c7(bM, FW, bRw, ad, fn, lQ, bGD, A, sp, ff, aEW, hp, en, eE, cM, zV, cT, but, z, Km, bim, be); #c1(SPINK5) c2(NP_001121171) c3(serine protease inhibitor Kazal-type 5 isoform c precursor) c4(45337) c5( ) c6( ) c7(bM, FW, bRw, ad, fn, lQ, bGD, A, sp, ff, aEW, hp, en, eE, cM, zV, cT, buT, z, Km, bim, be); #c1(SPINK5) c2(NP_006837) c3(serine protease inhibitor Kazal-type 5 isoform b precursor) c4(168545) c5(168546, 168547, 168548, 168549, 168550, 168551) c6( ) c7(bM, FW, bRw, ad, fn, lQ, bGD, A, sp, ff, aEW, hp, en, eE, cM, zV, cT, buT, z, Km, bim, be); #c1(SPINK6) c2(NP_001182219) c3(serine protease inhibitor Kazal-type 6 precursor) c4(74701, 74702) c5(74703, 74704, 74705, 74706, 74707, 74708, 74709, 74710, 74711, 74712, 74713, 74714, 74715, 74716) c6(74717, 74718) c7(eE, RJ, zg); #c1(SPINK7) c2(NP_115955) c3(serine protease inhibitor Kazal-type 7 precursor) c4(224920) c5(224921, 224922, 224923, 224924, 224925, 224926, 224927) c6(224928) c7(T, aC, ff, ht, it, ci, en, hu, g, bz, B); #c1(SPINT1) c2(NP_001027539) c3(kunitz-type protease inhibitor 1 isoform 2 precursor) c4(30251) c5(30252, 30253, 30254, 30255, 30256, 30257, 30258, 30259, 30260, 30261, 30262, 30263, 30264, 30265) c6(30266, 30267, 30268, 30269) c7(T, al, aA, sM, Bm, bB, fr, ci, q, ad, amg, aE, ds, gd, aT, dL, ae, eL, jf, d, s, aW, ff, hl, aq, aF, Fq, en, cl, t, g, op, gk, bE, wE, vA, gX, hL, at, m, vi); #c1(SPINT1) c2(XP_006720720) c3(kunitz-type protease inhibitor 1 isoform X1) c4(293622, 293623) c5(293624, 293625, 293626, 293627, 293628, 293629, 293630, 293631, 293632, 293633, 293634, 293635, 293636, 293637, 293638, 293639) c6(293640, 293641, 293642, 293643) c7(T, al, aA, sM, Bm, bB, fr, ci, q, ad, amg, aE, ds, gd, aT, dL, ae, eL, jf, d, s, aW, ff, hl, aq, aF, Fq, en, cl, t, g, op, gk, bE, wE, vA, gX, hL, at, m, vi); #c1(SPINT1) c2(XP_011520260) c3(kunitz-type protease inhibitor 1 isoform X3) c4( ) c5(315098) c6( ) c7(T, al, aA, sM, Bm, bB, fr, ci, q, ad, amg, aE, ds, gd, aT, dl, ae, eL, jf, d, s, aW, ff, hl, aq, aF, Fq, en, cl, t, g, op, gk, bE, wE, vA, gX, hL, at, m, vi); #c1(SPINT2) c2(NP_001159575) c3(kunitz-type protease inhibitor 2 isoform b precursor) c4(64302) c5(64303, 64304, 64305, 64306, 64307, 64308) c6( ) c7(T, jv, aMW, if, ha, aA, Bm, fr, ci, qN, bmx, zP, q, ad, eA, ajL, py, aE, amg, aT, ble, ae, jf, le, zQ, d, aW, blU, aq, aF, g, cl, as, ajB, t, op, xd, asl, vA, hL, clG, m, B); #c1(SPINT2) c2(NP_066925) c3(kunitz-type protease inhibitor 2 isoform a precursor) c4(207515) c5(207516, 207517, 207518, 207519, 207520, 207521) c6( ) c7(T, jv, aMW, if, ha, aA, Bm, fr, ci, qN, bmx, zP, q, ad, eA, ajL, py, aE, amg, aT, ble, ae, jf, le, zl, d, aW, blU, aq, aF, g, cl, as, ajB, t, op, xd, asl, vA, hL, clG, m, B); #c1 (SPNS1) c2(NP_001135921) c3(protein spinster homolog 1 isoform 4) c4(53559) c5(53560, 53561, 53562, 53563, 53564) c6( ) c7(d, aaU, z, ff, h, en, ae, eW, ad); #c1(SPNS1) c2(NP_001135922) c3(protein spinster homolog 1 isoform 2) c4(53565) c5(53566, 53567, 53568, 53569, 53570) c6( ) c7(d, aaU, z, ff, h, en, ae, eW, ad); #c1(SPNS1) c2(NP_00135923) c3(protein spinster homolog 1 isoform 3) c4(53571) c5(53572, 53573, 53574, 53575, 53576) c6( ) c7(d, aaU, z, ff, h, en, ae, eW, ad); #c1(SPNS1) c2(NP_114427) c3(protein spinster homolog 1 isoform 1) c4(222699) c5(222700, 222701, 222702, 222703, 222704, 222705, 222706) c6( ) c7(d, aaU, z, ff, h, en, ae, eW, ad); #c1(SPNS1) c2(XP_006721159) c3(protein spinster homolog 1 isoform X2) c4( ) c5(293887, 293888) c6( ) c7(d, aaU, z, ff, h, en, ae, eW, ad); #c1(SPNS2) c2(NP_001118230) c3(protein spinster homolog 2) c4(44047) c5(44048, 44049, 44050, 44051, 44052, 44053) c6( ) c7(md); #c1(SPO11) c2(NP_036576) c3(meiotic recombination protein SPO11 isoform a) c4(174471) c5(174472, 174473, 174474, 174475, 174476, 174477) c6( ) c7(vH, bt, gU, ND, il); #c1(SPO11) c2(NP_937998) c3(meiotic recombination protein SPO11 isoform b) c4(259485, 259486) c5(259487, 259488, 259489, 259490, 259491, 259492, 259493, 259494, 259495, 259496, 259497, 259498) c6( ) c7(vH, bt, gU, ND, il); #c1(SPO11) c2(XP_005260438) c3(meiotic recombination protein SPO11 isoform X3) c4(276540) c5(276541, 276542, 276543, 276544, 276545, 276546) c6( ) c7(vH, bt, gU, ND, il); #c1(SPOCK1) c2(NP_004589) c3(testican-1 precursor) c4(147944, 147945) c5(147946, 147947, 147948, 147949, 147950, 147951, 147952, 147953, 147954, 147955, 147956, 147957, 147958, 147959, 147960, 147961, 147962, 147963, 147964, 147965, 147966, 147967, 147968) c6(147969, 147970, 147971, 147972, 147973, 147974) c7(aC, d, eM, dH, aA, aj, kp, aF, B, ad); #c1(SPOCK2) c2(NP_001127906) c3(testican-2 isoform 1 precursor) c4(49487) c5(49488, 49489, 49490, 49491, 49492, 49493) c6( ) c7(mR, eA, aT); #c1(SPOCK2) c2(NP_055582) c3(testican-2 isoform 2 precursor) c4(182101, 182102) c5(182103, 182104, 182105, 182106, 182107, 182108, 182109, 182110, 182111, 182112, 182113, 182114) c6( ) c7(mR, eA, aT); #c1(SPOCK2) c2(XP_011538706) c3(testican-2 isoform X1) c4(336470) c5(336471, 336472, 336473, 336474, 336475, 336476) c6( ) c7(mR, eA, aT); #c1(SPOCK3) c2(NP_001035249) c3(testican-3 isoform 1 precursor) c4(32666) c5(32667, 32668, 32669, 32670, 32671, 32672, 32673, 32674, 32675, 32676, 32677, 32678, 32679, 32680, 32681, 32682, 32683, 32684, 32685, 32686) c6( ) c7(je, iM, kH); #c1(SPOCK3) c2(NP_001191281) c3(testican-3 isoform 3) c4(81271) c5(81272, 81273, 81274, 81275, 81276, 81277, 81278) c6( ) c7(je, iM, KH); #c1(SPOCK3) c2(NP_001191282) c3(testican-3 isoform 4) c4( ) c5(81279, 81280, 81281, 81282, 81283, 81284, 81285) c6( ) c7(je, iM, KH); #c1(SPOCK3) c2(NP_001191283) c3(testican-3 isoform 5) c4( ) c5(81286) c6( ) c7(je, iM, kH); #c1(SPOCK3) c2(NP_001191284) c3(testican-3 isoform 6) c4(81287, 81288) c5(81289, 81290, 81291, 81292, 81293, 81294, 81295, 81296) c6( ) c7(je, iM, kH); #c1(SPOCK3) c2(NP_001191285) c3(testican-3 isoform 7 precursor) c4(81297, 81298) c5(81299, 81300, 81301, 81302, 81303, 81304, 81305, 81306, 81307, 81308, 81309, 81310, 81311, 81312, 81313, 81314) c6( ) c7(je, iM, kH); #c1(SPOCK3) c2(NP_001191287) c3(testican-3 isoform 9 precursor) c4(81315, 81316) c5(81317, 81318, 81319, 81320, 81321, 81322, 81323, 81324, 81325, 81326, 81327, 81328, 81329, 81330, 81331, 81332) c6( ) c7(je, iM, KH); #c1(SPOCK3) c2(NP_001238896) c3(testican-3 isoform 11 precursor) c4(88016, 88017) c5(88018, 88019, 88020, 88021, 88022, 88023, 88024, 88025, 88026, 88027, 88028, 88029, 88030, 88031, 88032, 88033, 88034, 88035, 88036, 88037, 88038, 88039) c6( ) c7(je, iM, KH); #c1(SPOCK3) c2(NP_058646) c3(testican-3 isoform 2 precursor) c4(192949, 192950) c5(192951, 192952, 192953, 192954, 192955, 192956, 192957, 192958, 192959, 192960, 192961, 192962, 192963, 192964, 192965, 192966, 192967, 192968, 192969, 192970, 192971, 192972, 192973, 192974, 192975, 192976, 192977, 192978, 192979, 192980, 192981, 192982, 192983, 192984, 192985, 192986, 192987, 192988, 192989, 192990) c6( ) c7(je, iM, kH); #c1(SPON1) c2(NP_006099) c3(spondin-1 precursor) c4(161868, 161869) c5(161870, 161871, 161872, 161873, 161874, 161875, 161876, 161877, 161878, 161879, 161880, 161881, 161882, 161883) c6(161884, 161885) c7(ci, m); #c1(SPON2) c2(NP_001185950) c3(spondin-2 precursor) c4(76805, 76806, 76807, 76808, 76809, 76810, 76811) c5(76812, 76813, 76814, 76815, 76816, 76817, 76818, 76819, 76820, 76821, 76822, 76823, 76824, 76825, 76826, 76827, 76828, 76829, 76830, 76831, 76832, 76833, 76834, 76835, 76836, 76837, 76838, 76839, 76840, 76841, 76842, 76843, 76844, 76845) c6( ) c7(bL, sp, mQ, bz, B, ad); #c1(SPOP) c2(NP_003554) c3(speckle-type POZ protein) c4(139069, 139070, 139071, 139072, 139073, 139074, 139075, 139076, 139077, 139078, 139079) c5(139080, 139081, 139082, 139083, 139084, 139085, 139086, 139087, 139088, 139089, 139090, 139091, 139092, 139093, 139094, 139095, 139096, 139097, 139098, 139099, 139100, 139101, 139102, 139103, 139104, 139105, 139106, 139107, 139108, 139109, 139110, 139111, 139112, 139113, 139114, 139115, 139116, 139117, 139118, 139119, 139120, 139121, 139122, 139123, 139124, 139125, 139126, 139127, 139128, 139129, 139130, 139131, 139132, 139133, 139134, 139135, 139136, 139137, 139138, 139139, 139140, 139141, 139142, 139143, 139144, 139145, 139146, 139147, 139148, 139149, 139150, 139151, 139152, 139153, 139154, 139155, 139156, 139157, 139158, 139159, 139160) c6( ) c7(es, T, iO, py, iA, Ub, go, hL, aT, fr, ae, cd, ad); #c1(SPP1) c2(NP_000573) c3(osteopontin isoform OPN-b precursor) c4(12825) c5(12826, 12827, 12828, 12829, 12830, 12831) c6( ) c7(oO, bM, T, aC, fR, tQ, apr, fV, ea, e, al, if, fd, bB, cL, fo, ci, mF, Qp, ip, x, bv, dY, aE, Ma, aDk, bW, Qk, M, iv, K, qM, hu, wt, dF, cp, aa, xQ, ZH, up, dH, LC, aW, Ls, az, XV, hl, mQ, HA, uY, lh, fc, g, as, eF, bw, mK, zV, iP, y, eM, nB, iA, mx, k, m, lS, hh, fy, act, YS, iu, cll, dD, zt, Or, ht, aw, qy, n, aj, qN, zP, cd, ai, baA, ke, Bl, ft, jz, zw, Xe, h, alO, hn, jl, ey, bnv, nP, bmc, Dg, A, CN, anP, hQ, fQ, sm, bQc, Vt, aq, kp, ks, gG, fe, cM, wU, vU, ar, dB, GC, ajd, hL, at, E, bm, eo, fL, it, B, fl, vQ, bL, Dw, dA, eR, jp, DD, fG, jx, aA, cz, wo, hy, v, j, OY, C, sl, acn, lu, V, q, J, o, qH, eA, eJ, aT, Di, cy, bq, kU, ae, af, eu, bA, CB, ak, Uf, kl, bf, s, nO, iZ, Y, ev, ate, r, lo, en, cl, t, bNt, pb, ov, cT, QG, acW, ac, c, nM, iM, hc, nG, Da, dy, fl, fD, bt, awa, VB, hS, Mb, kh, BD, ob, aZ, aD, vr, wV, dG, eq, jE, mC, ad, hX, dP, aHF, av, F, fX, ib, py, akJ, hD, vw, Dj, ji, fb, vP, il, sj, uQ, TC, d, BN, je, qt, ff, xs, eH, cr, eZ, dN, gE, si, Vl, Fq, aF, iR, iV, Bu, bz, qa, LB, cH, D, baa, Bv, es, zr, Ew, yE, bE, X, gZ, cP, be, hE, aAN, ne, atd); #c1(SPP1) c2(NP_001035147) c3(osteopontin isoform OPN-a precursor) c4(32517, 32518) c5(32519, 32520, 32521, 32522, 32523, 32524, 32525, 32526, 32527, 32528, 32529, 32530) c6( ) c7(oO, bM, T, aC, fR, tQ, apr, fV, ea, e, al, if, fd, bB, cL, fo, ci, mF, Qp, ip, x, bv, dY, aE, Ma, aDk, bW, Qk, M, iv, K, qM, hu, wt, dF, cp, aa, xQ, ZH, up, dH, LC, aW, Ls, az, XV, hl, mQ, HA, uY, lh, fc, g, as, eF, bw, mK, zV, iP, y, eM, nB, iA, mx, k, m, lS, hh, fy, act, YS, iu, cll, dD, zt, Or, ht, aw, qy, n, aj, qN, zP, cd, ai, baA, ke, Bl, ft, jz, zw, Xe, h, alO, hn, jl, ey, bnv, nP, bmc, Dg, A, CN, anP, hQ, fQ, sm, bQc, Vt, aq, kp, ks, gG, fe, cM, wU, vU, ar, dB, GC, ajd, hL, at, E, bm, eo, fL, it, B, fl, vQ, bL, Dw, dA, eR, jp, DD, fG, jx, aA, cz, wo, hy, v, j, OY, C, sl, acn, lu, V, q, J, o, qH, eA, eJ, aT, Di, cy, bq, KU, ae, af, eu, bA, CB, ak, Uf, kl, bf, s, nO, iZ, Y, ev, ate, r, lo, en, cl, t, bNt, pb, ov, cT, QG, acW, ac, c, nM, iM, hc, nG, Da, dy, fl, fD, bt, awa, VB, hS, Mb, kh, BD, ob, aZ, aD, vr, wV, dG, eq, jE, mC, ad, hX, dP, aHF, av, F, fX, ib, py, akJ, hD, vw, Dj, ji, fb, vP, il, sj, uQ, TC, d, BN, je, qt, ff, xs, eH, cr, eZ, dN, gE, si, Vl, Fq, aF, iR, iV, Bu, bz, qa, LB, clH, D, baa, Bv, es, zr, Ew, yE, bE, X, gZ, cP, be, hE, aAN, ne, atd); #c1(SPP1) c2(NP_001035149) c3(osteopontin isoform OPN-c precursor) c4(32538) c5(32539, 32540, 32541, 32542, 32543, 32544) c6( ) c7(oO, bM, T, aC, fR, tQ, apr, fV, ea, e, al, if, fd, bB, cL, fo, ci, mF, Qp, ip, x, bv, dY, aE, Ma, aDk, bW, Qk, M, iv, K, qM, hu, wt, dF, cp, aa, xQ, ZH, up, dH, LC, aW, Ls, az, XV, hl, md, HA, uY, lh, fc, g, as, eF, bw, mk, zV, iP, y, eM, nB, iA, mx, k, m, lS, hh, fy, act, YS, iu, cll, dD, zt, Or, ht, aw, qy, n, aj, qN, zP, cd, ai, baA, ke, Bl, ft, jz, zw, Xe, h, alO, hn, jl, ey, bnv, nP, bmc, Dg, A, CN, anP, hQ, fQ, sm, bQc, Vt, aq, kp, ks, gG, fe, cM, wU, vU, ar, dB, GC, ajd, hl, at, E, bm, eo, fl, it, B, fl, vQ, bL, Dw, dA, eR, jp, DD, fG, jx, aA, cz, wo, hy, v, j, OY, C, sl, acn, lu, V, q, J, o, qH, eA, eJ, aT, Di, cy, bq, KU, ae, af, eu, bA, CB, ak, Uf, kl, bf, s, no, iZ, Y, ev, ate, r, lo, en, cl, t, bNt, pb, ov, cT, QG, acW, ac, c, nM, iM, hc, nG, Da, dy, fl, fD, bt, awa, VB, hS, Mb, kh, BD, ob, aZ, aD, vr, wV, dG, eq, jE, mC, ad, hX, dP, aHF, av, F, fX, ib, py, akJ, hD, vw, Dj, ji, fb, vP, iL, sj, uQ, TC, d, BN, je, qt, ff, xs, eH, cr, eZ, dN, gE, si, Vl, Fq, aF, iR, iV, Bu, bz, qa, LB, clH, D, baa, Bv, es, zr, Ew, yE, bE, X, gZ, cP, be, hE, aAN, ne, atd); #c1(SPP1) c2(NP_001238758) c3(osteopontin isoform 4 precursor) c4(87893) c5(87894, 87895, 87896, 87897, 87898, 87899) c6( ) c7(oO, bM, T, aC, fR, tQ, apr, fV, ea, e, al, if, fd, bB, cL, fo, ci, mF, Qp, ip, x, bv, dY, aE, Ma, aDk, bW, Qk, M, iv, K, qM, hu, wt, dF, cp, aa, xQ, ZH, up, dH, LC, aW, Ls, az, XV, hl, mQ, HA, uY, lh, fc, g, as, eF, bw, mK, zV, iP, y, eM, nB, iA, mx, k, m, lS, hh, fy, act, YS, iu, cll, dD, zt, Or, ht, aw, qy, n, aj, qN, zP, cd, ai, baA, ke, Bl, ft, jz, zw, Xe, h, alO, hn, jl, ey, bnv, nP, bmc, Dg, A, CN, anP, hQ, fQ, sm, bQc, Vt, aq, kp, ks, gG, fe, cM, wU, vU, ar, dB, GC, ajd, hl, at, E, bm, eo, fl, it, B, fl, vQ, bL, Dw, dA, eR, jp, DD, fG, jx, aA, cz, wo, hy, v, j, OY, C, sl, acn, lu, V, q, J, o, qH, eA, eJ, aT, Di, cy, bq, KU, ae, af, eu, bA, CB, ak, Uf, kl, bf, s, no, iZ, Y, ev, ate, r, lo, en, cl, t, bNt, pb, ov, cT, QG, acW, ac, c, nM, iM, hc, nG, Da, dy, fl, fD, bt, awa, VB, hS, Mb, kh, BD, ob, aZ, aD, vr, wV, dG, eq, jE, mC, ad, hX, dP, aHF, av, F, fX, ib, py, akJ, hD, vw, Dj, ji, fb, vP, il, sj, uQ, TC, d, BN, je, qt, ff, xs, eH, cr, eZ, dN, gE, si, Vl, Fq, aF, iR, iV, Bu, bz, qa, LB, clH, D, baa, Bv, es, zr, Ew, yE, bE, X, gZ, cP, be, hE, aAN, ne, atd); #c1(SPP2) c2(NP_008875) c3(secreted phosphoprotein 24 precursor) c4(169429) c5(169430, 169431, 169432, 169433, 169434, 169435, 169436, 169437, 169438, 169439, 169440, 169441) c6( ) c7(bM, cr, bA); #c1(SPPL2A) c2(NP_116191) c3(signal peptide peptidase-like 2A precursor) c4(225666) c5(225667, 225668, 225669, 225670, 225671, 225672) c6( ) c7(Mb, T, hd, kP, WY, aW, eH, aep, aT, aaE, dL, as, eF, eL, B); #c1(SPPL2B) c2(NP_001070706) c3(signal peptide peptidase-like 28 isoform 3 precursor) c4(34779) c5(34780, 34781, 34782, 34783, 34784, 34785, 34786, 34787, 34788, 34789) c6( ) c7(hk, CW); #c1(SPPL2B) c2(NP_694533) c3(signal peptide peptidase-like 28 isoform 2 precursor) c4(245085) c5( ) c6( ) c7(hk, CW); #c1(SPPL2B) c2(XP_011526440) c3(signal peptide peptidase-like 2B isoform X1) c4( ) c5(320751, 320752) c6( ) c7(hk, CW); #c1(SPPL2B) c2(XP_011526442) c3(signal peptide peptidase-like 2B isoform X3) c4( ) c5(320753) c6( ) c7(hk, CW); #c1(SPPL2C) c2(NP_787078) c3(signal peptide peptidase-like 2C precursor) c4(252069, 252070) c5(252071, 252072, 252073, 252074, 252075, 252076, 252077, 252078, 252079, 252080, 252081, 252082) c6( ) c7(act, an); #c1(SPPL3) c2(NP_620584) c3(signal peptide peptidase-like 3) c4(237798) c5(237799, 237800, 237801, 237802, 237803, 237804, 237805, 237806, 237807, 237808, 237809, 237810) c6( ) c7(bL, T, au, d, dV); #c1(SPPL3) c2(XP_011536226) c3(signal peptide peptidase-like 3 isoform X1) c4(333543) c5( ) c6( ) c7(bL, T, au, d, dV); #c1(SPR) c2(NP_003115) c3(sepiapterin reductase) c4(135441) c5(135442, 135443, 135444, 135445, 135446, 135447) c6( ) c7(bN, bfb, aji, bAk, cA, clJ, aU, ctY, bK, an, Z, f, aWT, ae, bAl, ad); #c1(SPRED1) c2(NP_689807) c3(sprouty-related, EVH1 domain-containing protein 1) c4(244016) c5(244017, 244018, 244019, 244020, 244021, 244022) c6( ) c7(aBK, ly, T, lD, x, aeR, Ew, nX, aeO, aiz, k, eA, ae, B, ad, lx, uQ); #c1(SPRED2) c2(NP_001121682) c3(sprouty-related, EVH1 domain-containing protein 2 isoform b) c4(46041) c5(46042, 46043, 46044, 46045, 46046, 46047) c6( ) c7(cF, Ew, nX, rD, y, ae, B, ad); #c1(SPRED2) c2(XP_005264257) c3(sprouty-related, EVH1 domain-containing protein 2 isoform X2) c4(278737) c5( ) c6( ) c7(cF, Ew, nX, rD, y, ae, B, ad); #c1(SPRED2) c2(XP_005264259) c3(sprouty-related, EVH1 domain-containing protein 2 isoform X4) c4( ) c5(278738, 278739, 278740, 278741, 278742, 278743) c6( ) c7(cF, Ew, nX, rD, y, ae, B, ad); #c1(SPRED2) c2(XP_006712029) c3(sprouty-related, EVH1 domain-containing protein 2 isoform X1) c4(286346) c5(286347, 286348, 286349, 286350, 286351, 286352) c6( ) c7(cF, Ew, nX, rD, y, ae, B, ad); #c1(SPRED3) c2(XP_011525279) c3(sprouty-related, EVH1 domain-containing protein 3 isoform X1) c4(319729, 319730) c5(319731, 319732, 319733, 319734, 319735, 319736, 319737, 319738, 319739, 319740, 319741, 319742) c6( ) c7(nX, Ew); #c1(SPRED3) c2(XP_011525281) c3(sprouty-related, EVH1 domain-containing protein 3 isoform X3) c4( ) c5(319743) c6( ) c7(nX, Ew); #c1(SPRN) c2(NP_001012526) c3(shadow of prion protein precursor) c4(25040) c5(25041, 25042, 25043, 25044, 25045, 25046, 25047, 25048, 25049, 25050, 25051) c6( ) c7(dt, jX, dS); #c1(SPRR1A) c2(NP_001186757) c3(cornifin-A) c4(78512) c5(78513, 78514, 78515, 78516, 78517, 78518, 78519, 78520, 78521, 78522) c6( ) c7(fM, lg, ff, en, eE, it); #c1(SPRR1B) c2(NP_003116) c3(cornifin-B) c4(135448, 135449) c5(135450, 135451, 135452, 135453, 135454, 135455, 135456, 135457, 135458, 135459, 135460, 135461) c6( ) c7(Qy, rl, rM, K, g, lQ, eE); #c1(SPRR2A) c2(NP_005979) c3(small proline-rich protein 2A) c4(160946) c5(160947, 160948, 160949, 160950, 160951, 160952) c6( ) c7(aJJ, T, jz, xu, alC, eW, A, eD, qh, hl, jx, aA, aj, v, K, Pd, it, YK, J, aa); #c1(SPRR2B) c2(NP_001017418) c3(small proline-rich protein 2B) c4(26478) c5( ) c6( ) c7(bM); #c1(SPRR3) c2(NP_001091058) c3(small proline-rich protein 3) c4(38388, 38389, 38390) c5(38391, 38392, 38393, 38394, 38395, 38396, 38397, 38398, 38399, 38400, 38401, 38402, 38403, 38404, 38405, 38406, 38407, 38408, 38409, 38410, 38411, 38412) c6( ) c7(T, aEl, eE, ff, it, ci, K, Xk, lQ, hu, en, bzT, g); #c1(SPRTN) c2(NP_001010984) c3(sprT-like domain-containing protein Spartan isoform b) c4(24461) c5(24462, 24463, 24464, 24465, 24466, 24467) c6( ) c7(T, MR, rC, La, ci, K, B); #c1(SPRTN) c2(NP_001248391) c3(sprT-like domain-containing protein Spartan isoform c) c4(93936) c5(93937, 93938, 93939, 93940, 93941, 93942, 93943, 93944, 93945, 93946, 93947, 93948, 93949) c6( ) c7(T, MR, rC, La, ci, K, B); #c1(SPRTN) c2(NP_114407) c3(sprT-like domain-containing protein Spartan isoform a) c4(222593, 222594) c5(222595, 222596, 222597, 222598, 222599, 222600) c6( ) c7(T, MR, rC, La, ci, K, B); #c1(SPRTN) c2(XP_006711881) c3(sprT-like domain-containing protein Spartan isoform X1) c4(286277) c5(286278) c6( ) c7(T, MR, rC, La, ci, K, B); #c1(SPRTN) c2(XP_011542590) c3(sprT-like domain-containing protein Spartan isoform X2) c4( ) c5(341160, 341161, 341162, 341163, 341164, 341165) c6( ) c7(T, MR, rC, La, ci, K, B); #c1(SPRY1) c2(NP_001244968) c3(protein sprouty homolog 1) c4(92479, 92480, 92481, 92482) c5(92483, 92484, 92485, 92486, 92487, 92488, 92489, 92490, 92491, 92492, 92493, 92494, 92495, 92496, 92497, 92498, 92499, 92500, 92501, 92502, 92503, 92504, 92505, 92506, 92507, 92508, 92509, 92510) c6( ) c7(T, al, v, ad, x, bv, nv, nX, aeO, aT, ae, aMi, bA, aeR, Qr, aW, pm, t, Qt, gk, Ew, bE, k, Xu, B); #c1(SPRY2) c2(NP_005833) c3(protein sprouty homolog 2) c4(159748, 159749) c5(159750, 159751, 159752, 159753, 159754, 159755, 159756, 159757, 159758, 159759, 159760, 159761, 159762, 159763, 159764, 159765, 159766, 159767, 159768, 159769, 159770) c6( ) c7(bL, T, fM, wu, iW, al, jh, cz, ckQ, bB, aj, v, ci, eA, ad, Na, clk, av, hd, pN, nX, aT, rD, Nc, wm, ae, aa, eG, aW, Y, Az, aq, g, hf, cl, cm, iR, bz, bvU, Qt, gk, nM, Ew, eM, nB, bE, KY, B); #c1(SPRY3) c2(NP_005831) c3(protein sprouty homolog 3) c4(159734, 159735) c5(159736, 159737, 159738, 159739, 159740, 159741, 159742, 159743, 159744, 159745, 159746, 159747) c6( ) c7(nX, Ew); #c1(SPRY4) c2(NP_001280218) c3(protein sprouty homolog 4 isoform 2) c4(111467, 111468) c5(111469, 111470, 111471, 111472, 111473, 111474, 111475, 111476, 111477, 111478, 111479, 111480, 111481, 111482, 111483, 111484) c6( ) c7(T, clL, ac, eM, vT, Ew, nX, De, Y, Lf, Lb, j, EA, bu, pm, fb, bz, Ld, ad, cp); #c1(SPRY4) c2(NP_112226) c3(protein sprouty homolog 4 isoform 1) c4(220971, 220972) c5(220973, 220974, 220975, 220976, 220977, 220978, 220979, 220980) c6( ) c7(T, clL, ac, eM, vT, Ew, nX, oe, Y, Lf, Lb, j, EA, bu, pm, fb, bz, Ld, ad, cp); #c1(SPRYD7) c2(NP_001120954) c3(SPRY domain-containing protein 7 isoform 2) c4(44982) c5(44983, 44984, 44985, 44986, 44987, 44988) c6( ) c7(cgf); #c1(SPRYD7) c2(NP_065189) c3(SPRY domain-containing protein 7 isoform 1) c4(204501) c5(204502, 204503, 204504, 204505, 204506, 204507) c6( ) c7(cgf); #c1(SPSB3) c2(NP_543137) c3(SPRY domain-containing SOCS box protein 3) c4(233355, 233356) c5(233357, 233358, 233359, 233360, 233361, 233362, 233363, 233364, 233365, 233366, 233367, 233368, 233369, 233370, 233371, 233372) c6( ) c7(v); #c1(SPSB4) c2(NP_543138) c3(SPRY domain-containing SOCS box protein 4) c4(233373) c5(233374, 233375, 233376, 233377, 233378, 233379) c6( ) c7(wL, aj); #c1(SPTA1) c2(XP_011508218) c3(spectrin alpha chain, erythrocytic 1 isoform X1) c4(298839, 298840) c5(298841, 298842, 298843, 298844, 298845, 298846, 298847, 298848, 298849, 298850, 298851, 298852) c6( ) c7(os, VJ, HY, qb, Fm, clN, dV, cz, cr, jh, S, ci, pw, clM, qs); #c1 (SPTAN1) c2(NP_001123910) c3(spectrin alpha chain, non-erythrocytic 1 isoform 1) c4(47983) c5( ) c6( ) c7(kn, T, b, eZ, bE, aJ, i, ha, afi, oj, U, r, aA, clO, bB, fN, oZ, nA, bd, aF, cC); #c1(SPTAN1) c2(NP_001182461) c3(spectrin alpha chain, non-erythrocytic 1 isoform 3) c4(74948) c5(74949, 74950, 74951, 74952, 74953) c6( ) c7(kn, T, b, eZ, bE, aJ, i, ha, afi, oj, U, r, aA, clO, bB, fN, oZ, nA, bd, aF, cC); #c1(SPTAN1) c2(XP_006717308) c3(spectrin alpha chain, non-erythrocytic 1 isoform X1) c4(290892) c5( ) c6( ) c7(kn, T, b, eZ, bE, aJ, i, ha, afi, oj, U, r, aA, clO, bB, fN, oZ, nA, bd, aF, cC); #c1(SPTAN1) c2(XP_006717311) c3(spectrin alpha chain, non-erythrocytic 1 isoform X4) c4(290893) c5(290894, 290895, 290896, 290897, 290898, 290899, 290900, 290901, 290902, 290903, 290904, 290905, 290906) c6( ) c7(kn, T, b, eZ, bE, aJ, i, ha, afi, oj, U, r, aA, clO, bB, fN, oZ, nA, bd, aF, cC); #c1(SPTAN1) c2(XP_006717312) c3(spectrin alpha chain, non-erythrocytic 1 isoform X5) c4(290907) c5(290908, 290909, 290910, 290911, 290912, 290913) c6( ) c7(kn, T, b, eZ, bE, aJ, i, ha, afi, oj, U, r, aA, clO, bB, fN, oZ, nA, bd, aF, cC); #c1(SPTAN1) c2(XP_006717316) c3(spectrin alpha chain, non-erythrocytic 1 isoform X9) c4( ) c5(290914, 290915, 290916, 290917, 290918, 290919) c6( ) c7(kn, T, b, eZ, bE, aJ, i, ha, afi, oj, U, r, aA, clO, bB, fN, oZ, nA, bd, aF, cC); #c1(SPTB) c2(NP_000338) c3(spectrin beta chain, erythrocytic isoform b) c4(7398, 7399) c5(7400, 7401, 7402, 7403, 7404, 7405, 7406, 7407, 7408, 7409) c6( ) c7(VJ, ac, c, Fm, vA, oj, S, clP, eg, qs, eu, clQ, ald); #c1(SPTB) c2(NP_001020029) c3(spectrin beta chain, erythrocytic isoform a) c4(28080, 28081, 28082) c5(28083, 28084, 28085, 28086, 28087, 28088, 28089, 28090, 28091, 28092, 28093, 28094, 28095, 28096, 28097, 28098, 28099, 28100, 28101, 28102, 28103, 28104, 28105, 28106, 28107, 28108, 28109, 28110) c6( ) c7(VJ, ac, c, Fm, vA, oj, S, clP, eg, qs, eu, clQ, ald); #c1(SPTBN1) c2(NP_842565) c3(spectrin beta chain, non-erythrocytic 1 isoform 2) c4(253636) c5(253637, 253638, 253639, 253640, 253641, 253642) c6( ) c7(T, bE, Bj, ml, gX, r, aA, bB, dz, fN, aF, pw, bt, q, bA); #c1(SPTBN1) c2(XP_006712150) c3(spectrin beta chain, non-erythrocytic 1 isoform X1) c4(286447, 286448) c5(286449, 286450, 286451, 286452, 286453, 286454, 286455, 286456, 286457, 286458, 286459, 286460) c6( ) c7(T, bE, Bj, ml, gX, r, aA, bB, dz, fN, aF, pw, bt, q, bA); #c1(SPTBN2) c2(NP_008877) c3(spectrin beta chain, non-erythrocytic 2) c4( ) c5(169442, 169443, 169444, 169445, 169446, 169447, 169448, 169449, 169450, 169451, 169452, 169453, 169454) c6( ) c7(Ng, alM, bM, BC, ky, KB, aDy, Kv, clR, U, rf, yZ, EH, cC); #c1(SPTBN2) c2(XP_005274250) c3(spectrin beta chain, non-erythrocytic 2 isoform X1) c4(285006, 285007, 285008, 285009) c5(285010, 285011, 285012, 285013, 285014, 285015, 285016, 285017, 285018, 285019, 285020, 285021, 285022, 285023, 285024, 285025) c6( ) c7(Ng, alM, bM, BC, ky, kB, aDy, Kv, clR, U, rf, yZ, EH, cC); #c1(SPTBN4) c2(NP_066022) c3(spectrin beta chain, non-erythrocytic 4 isoform signal) c4(206752, 206753, 206754, 206755) c5(206756, 206757, 206758, 206759, 206760, 206761, 206762, 206763, 206764, 206765, 206766, 206767, 206768, 206769, 206770, 206771, 206772, 206773, 206774, 206775, 206776, 206777, 206778, 206779, 206780, 206781, 206782, 206783, 206784, 206785, 206786) c6( ) c7(kl, fr, ac); #c1(SPTBN4) c2(NP_079489) c3(spectrin beta chain, non-erythrocytic 4 isoform sigma5) c4(218335) c5(218336, 218337, 218338, 218339, 218340) c6( ) c7(kl, fr, ac); #c1(SPTBN4) c2(XP_011525475) c3(spectrin beta chain, non-erythrocytic 4 isoform X2) c4(319888) c5( ) c6( ) c7(kl, fr, ac); #c1(SPTBN5) c2(NP_057726) c3(spectrin beta chain, non-erythrocytic 5) c4(192533) c5(192534, 192535, 192536, 192537, 192538, 192539, 192540) c6( ) c7(ac); #c1(SPTLC1) c2(NP_006406) c3(serine palmitoyltransferase 1 isoform a) c4(164757, 164758) c5(164759, 164760, 164761, 164762, 164763, 164764) c6( ) c7(T, Zm, bb, ql, xU, Pt, aFa, bT, xi, U, bhc, dr, j, fu, bX, bU, cm, aen, af, aR); #c1(SPTLC1) c2(NP_847894) c3(serine palmitoyltransferase 1 isoform b) c4( ) c5(253650, 253651, 253652, 253653, 253654, 253655) c6( ) c7(T, Zm, bb, ql, xU, Pt, aFa, bT, xi, U, bhc, dr, j, fu, bX, bU, cm, aen, af, aR); #c1(SPTLC2) c2(NP_004854) c3(serine palmitoyltransferase 2) c4(150317) c5(150318, 150319, 150320, 150321, 150322, 150323) c6( ) c7(xU, Pt, clS, Hn, n, blY, fu); #c1(SPTLC3) c2(NP_060797) c3(serine palmitoyltransferase 3) c4(197881, 197882, 197883, 197884) c5(197885, 197886, 197887, 197888, 197889, 197890) c6( ) c7(beA, U); #c1(SPTLC3) c2(XP_011527581) c3(serine palmitoyltransferase 3 isoform X2) c4(322430) c5(322431, 322432, 322433, 322434, 322435, 322436, 322437, 322438, 322439, 322440, 322441, 322442, 322443, 322444, 322445, 322446, 322447, 322448, 322449, 322450, 322451, 322452, 322453, 322454) c6( ) c7(beA, U); #c1(SPTSSB) c2(NP_001035189) c3(serine palmitoyltransferase small subunit B) c4(32565) c5(32566, 32567, 32568, 32569, 32570, 32571, 32572, 32573, 32574) c6( ) c7(fq); #c1(SPTSSB) c2(XP_011510784) c3(serine palmitoyltransferase small subunit B isoform X1) c4( ) c5(301193) c6( ) c7(fq); #c1(SPTY2D1) c2(NP_919261) c3(protein SPT2 homolog) c4(258333, 258334) c5(258335, 258336, 258337, 258338, 258339, 258340, 258341, 258342, 258343, 258344, 258345, 258346) c6( ) c7(j, ac); #c1(SPZ1) c2(NP_115956) c3(spermatogenic leucine zipper protein 1) c4(224929, 224930) c5(224931, 224932, 224933, 224934, 224935, 224936, 224937, 224938, 224939, 224940, 224941, 224942) c6( ) c7(T, Tb, DD, aZ, if, e, aA, bB, fd, v, lR, ci, qN, fr, cd, ad, ai, eA, DJ, Dq, aT, cy, ae, cp, jf, eG, asp, s, nO, aW, Y, oV, ff, aF, en, cl, dw, aaO, cm, bz, pb, bfP, DT, eM, iA, bE, hl, agf, mf, m, y, iu); #c1(SQLE) c2(NP_003120) c3(squalene monooxygenase) c4(135462) c5(135463, 135464, 135465, 135466, 135467, 135468, 135469) c6( ) c7(jp, cH, aW, aT, aq, m, t, ae, B, dr, ad); #c1(SQLE) c2(XP_011515548) c3(squalene monooxygenase isoform X1) c4(308374, 308375) c5(308376, 308377, 308378, 308379, 308380, 308381, 308382, 308383, 308384, 308385, 308386) c6( ) c7(jp, cH, aW, al, aq, m, t, ae, B, dr, ad); #c1(SQRDL) c2(NP_067022) c3(sulfide:quinone oxidoreductase, mitochondrial) c4(208455) c5(208456, 208457, 208458, 208459, 208460, 208461, 208462, 208463, 208464, 208465) c6( ) c7(fe, fq, ad, an); #c1(SQSTM1) c2(NP_001135771) c3(sequestosome-1 isoform 2) c4( ) c5(53186, 53187, 53188, 53189, 53190, 53191, 53192, 53193, 53194, 53195, 53196, 53197, 53198, 53199, 53200, 53201, 53202, 53203, 53204, 53205, 53206, 53207, 53208, 53209, 53210, 53211, 53212) c6( ) c7(bM, T, fM, Ol, if, jh, GC, To, v, Vd, ci, BF, aue, q, ad, eA, eJ, Kx, jz, py, aE, zw, WY, dE, oj, aT, bdM, h, K, an, Wr, nJ, ae, af, bc, d, mR, il, aW, Y, ff, az, alR, eH, r, aq, aaE, dg, kp, CO, en, g, eF, f, cm, ik, es, bb, kP, c, W, eM, A, U, Kj, EY, bdR, bi, zu, bt, xg, B); #c1(SQSTM1) c2(NP_003891) c3(sequestosome-1 isoform 1) c4(141905, 141906, 141907, 141908, 141909) c5(141910, 141911, 141912, 141913, 141914, 141915, 141916, 141917, 141918, 141919, 141920) c6( ) c7(bM, T, fM, Ol, if, jh, GC, To, v, Vd, ci, BF, aue, q, ad, eA, eJ, Kx, jz, py, aE, zw, WY, dE, oj, aT, bdM, h, K, an, Wr, nJ, ae, af, bc, d, mR, il, aW, Y, ff, az, alR, eH, r, aq, aaE, dg, kp, CQ, en, g, eF, f, cm, ik, es, bb, kP, c, W, eM, iA, U, Kj, EY, bdR, bi, zu, bt, xg, B); #c1(SRA1) c2(NP_001030312) c3(steroid receptor RNA activator 1 isoform 1) c4(30998) c5(30999, 31000, 31001, 31002, 31003, 31004, 31005) c6( ) c7(T, axd, n, oH, ci, aNe, ad, aR, cF, ws, aT, dL, jj, XZ, ae, af, eL, xQ, pr, aW, fc, aq, EZ, cl, t, acD, bz, ck, aNd, at, mf, np, m, cP, kS, B); #c1(SRBD1) c2(NP_060549) c3(SI RNA-binding domain-containing protein 1) c4(196515, 196516) c5(196517, 196518, 196519, 196520, 196521, 196522, 196523, 196524, 196525, 196526, 196527, 196528) c6( ) c7(dw, e, HQ, dH); #c1(SRC) c2(NP_005408) c3(proto-oncogene tyrosine-protein kinase Src) c4(155781, 155782) c5(155783, 155784, 155785, 155786, 155787, 155788, 155789, 155790, 155791, 155792, 155793, 155794, 155795, 155796, 155797, 155798, 155799, 155800, 155801, 155802, 155803, 155804, 155805, 155806) c6( ) c7(bL, T, dD, aC, ya, ow, azi, fG, zs, jh, aA, cz, bB, eq, ci, AO, iJ, q, ad, eA, zn, hX, eJ, iu, ft, ib, aE, afb, yt, pX, oW, aT, cU, lG, Ou, dL, h, ox, ji, fb, pq, ae, eu, VC, gj, d, B, iU, qt, aW, Y, ff, r, aq, aF, en, cl, as, t, cm, bz, Qt, bN, asE, ck, D, ac, eM, nB, dB, bE, bP, cj, at, pg, m, adg, zu, bt, cB, X, hS); #c1(SRCAP) c2(NP_006653) c3(helicase SRCAP) c4(166912, 166913, 166914, 166915) c5(166916, 166917, 166918, 166919, 166920, 166921, 166922, 166923, 166924, 166925, 166926, 166927, 166928, 166929, 166930, 166931, 166932, 166933, 166934, 166935, 166936, 166937, 166938, 166939, 166940, 166941, 166942, 166943, 166944, 166945) c6( ) c7(ck, qK, all, aVB, ae, ad); #c1(SRCIN1) c2(NP_079524) c3(SRC kinase signaling inhibitor 1) c4(218597, 218598, 218599) c5(218600, 218601, 218602, 218603, 218604, 218605, 218606, 218607, 218608, 218609, 218610, 218611, 218612, 218613, 218614, 218615, 218616, 218617) c6( ) c7(ac, il, eZ, aT, bz, iu); #c1(SRD5A1) c2(XP_011512405) c3(3-oxo-5-alpha-steroid 4-dehydrogenase 1 isoform X1) c4(303092, 303093) c5(303094, 303095, 303096, 303097, 303098, 303099, 303100, 303101, 303102, 303103, 303104) c6( ) c7(bL, G, T, aZ, iW, adW, n, v, Fb, ci, Ns, bX, avO, eA, ad, dP, aex, il, dv, aT, PD, uV, ae, ael, kl, bf, Ln, aW, fc, PA, Pp, g, PF, bN, pu, kP, ag, aez, m, cP, gT, B); #c1(SRD5A2) c2(XP_011531370) c3(3-oxo-5-alpha-steroid 4-dehydrogenase 2 isoform X1) c4( ) c5(327376) c6( ) c7(bL, G, T, aZ, bbn, jx, adW, n, aj, Ns, bX, avO, ad, dP, aej, F, aex, vJ, aT, UD, ae, af, aeQ, ael, yi, kl, UL, Ln, Ub, aW, hl, aq, Pp, cl, iR, t, bN, gk, pu, UB, hL, aez, aeM, gv, m, B); #c1(SRD5A3) c2(NP_078868) c3(polyprenol reductase) c4(215847) c5(215848, 215849, 215850, 215851, 215852, 215853) c6( ) c7(clU, ky, dv, Km, nA, oQ, clT, ae, LS, ad); #c1(SREBF1) c2(NP_001005291) c3(sterol regulatory element-binding protein 1 isoform a) c4(21322, 21323, 21324) c5(21325, 21326, 21327, 21328, 21329, 21330, 21331, 21332, 21333, 21334, 21335, 21336, 21337) c6( ) c7(bL, T, dD, fM, tS, eR, aoq, ea, jx, cz, aj, v, j, eq, aY, gb, q, J, st, GK, ad, hX, LL, uT, ws, AJ, rC, aT, h, jy, K, bq, de, an, ae, af, hi, A, d, cH, aW, aBq, fc, r, dq, aq, cl, GR, JP, au, bo, hL, dy, Fw, cP, du, ei, B, vQ); #c1(SREBF1) c2(NP_004167) c3(sterol regulatory element-binding protein 1 isoform b) c4(144014, 144015, 144016, 144017) c5(144018, 144019, 144020, 144021, 144022, 144023, 144024, 144025, 144026, 144027, 144028, 144029, 144030, 144031, 144032, 144033, 144034, 144035, 144036) c6( ) c7(bL, T, dD, fM, tS, eR, aoq, ea, jx, cz, aj, v, j, eq, aY, gb, q, J, st, GK, ad, hX, LL, uT, ws, QJ, rC, aT, h, jy, K, bq, de, an, ae, af, hi, A, d, cH, aW, aBq, fc, r, dq, aq, cl, GR, JP, au, bo, hL, dy, Fw, cP, du, ei, B, vQ); #c1(SREBF1) c2(XP_005256829) c3(sterol regulatory element-binding protein 1 isoform X1) c4(274549) c5( ) c6( ) c7(bL, T, dD, fM, tS, eR, aoq, ea, jx, cz, aj, v, j, eq, aY, gb, q, J, st, GK, ad, hX, LL, uT, ws, QJ, rC, aT, h, jy, K, bq, de, an, ae, af, hi, A, d, cH, aW, aBq, fc, r, dq, aq, cl, GR, JP, au, bo, hl, dy, Fw, cP, du, ei, B, vQ); #c1(SREBF1) c2(XP_006721633) c3(sterol regulatory element-binding protein 1 isoform X2) c4(294120) c5(294121, 294122, 294123, 294124, 294125, 294126, 294127, 294128, 294129, 294130, 294131, 294132, 294133, 294134, 294135, 294136, 294137) c6( ) c7(bL, T, dD, fM, tS, eR, aoq, ea, jx, cz, aj, v, j, eq, aY, gb, q, J, st, GK, ad, hX, LL, uT, ws, QJ, rC, aT, h, jy, K, bq, de, an, ae, af, hi, A, d, cH, aW, aBq, fc, r, dq, aq, cl, GR, JP, au, bo, hl, dy, Fw, cP, du, ei, B, vQ); #c1(SREBF1) c2(XP_011522300) c3(sterol regulatory element-binding protein 1 isoform X3) c4(316973) c5(316974, 316975, 316976, 316977, 316978, 316979) c6( ) c7(bL, T, dD, fM, tS, eR, aoq, ea, jx, cz, aj, v, j, eq, aY, gb, q, J, st, GK, ad, hX, LL, uT, ws, QJ, rC, aT, h, jy, K, bq, de, an, ae, af, hi, A, d, cH, aW, aBq, fc, r, dq, aq, cl, GR, JP, au, bo, hl, dy, Fw, cP, du, ei, B, vQ); #c1(SREBF1) c2(XP_011522301) c3(sterol regulatory element-binding protein 1 isoform X4) c4( ) c5(316980, 316981, 316982, 316983, 316984, 316985) c6( ) c7(bL, T, dD, fM, tS, eR, aoq, ea, jx, cz, aj, v, j, eq, aY, gb, q, J, st, GK, ad, hX, LL, uT, ws, QJ, rC, aT, h, jy, K, bq, dQ, an, ae, af, hi, A, d, cH, aW, aBq, fc, r, dq, aq, cl, GR, JP, au, bo, hL, dy, Fw, cP, du, ei, B, vQ); #c1(SREBF2) c2(NP_004590) c3(sterol regulatory element-binding protein 2) c4(147975, 147976, 147977) c5(147978, 147979, 147980, 147981, 147982, 147983, 147984, 147985, 147986, 147987, 147988, 147989, 147990, 147991, 147992, 147993, 147994, 147995, 147996) c6( ) c7(T, dD, ZP, eR, ea, aw, aA, cz, aj, v, j, eq, aY, gb, J, ad, dY, aT, h, dL, K, dQ, ae, af, eL, bFd, cH, cW, aW, hl, r, cm, iO, cO, gZ, nT, mf, cS, m, dy, du, va, ei, B, ek); #c1(SREBF2) c2(XP_011528649) c3(sterol regulatory element-binding protein 2 isoform X2) c4(323924) c5(323925, 323926, 323927, 323928, 323929, 323930) c6( ) c7(T, dD, ZP, eR, ea, aw, aA, cz, aj, v, j, eq, aY, gb, J, ad, dY, aT, h, dL, K, dQ, ae, af, eL, bFd, cH, cW, aW, hl, r, cm, iO, cO, gZ, nT, mf, cS, m, dy, du, va, ei, B, ek); #c1(SREK1) c2(NP_001070667) c3(splicing regulatory glutamine/lysine-rich protein 1 isoform a) c4(34767) c5( ) c6( ) c7(af); #c1(SREK1) c2(NP_001257421) c3(splicing regulatory glutamine/lysine-rich protein 1 isoform c) c4(95415) c5(95416, 95417, 95418, 95419, 95420) c6( ) c7(af); #c1(SREK1) c2(NP_631907) c3(splicing regulatory glutamine/lysine-rich protein 1 isoform b) c4(238303) c5( ) c6( ) c7(af); #c1(SREK1) c2(XP_005248499) c3(splicing regulatory glutamine/lysine-rich protein 1 isoform X5) c4( ) c5(268430, 268431, 268432, 268433, 268434, 268435) c6( ) c7(af); #c1(SREK1) c2(XP_011541472) c3(splicing regulatory glutamine/lysine-rich protein 1 isoform X3) c4(340051) c5(340052, 340053, 340054, 340055, 340056, 340057, 340058, 340059, 340060, 340061, 340062, 340063, 340064) c6( ) c7(af); #c1(SREK1IP1) c2(NP_776190) c3(protein SREK1IP1) c4(250964) c5(250965, 250966, 250967, 250968, 250969) c6( ) c7(af); #c1(SRF) c2(NP_001278930) c3(serum response factor isoform 2) c4( ) c5(111058, 111059, 111060, 111061, 111062) c6( ) c7(T, aCh, AS, Am, ha, Xi, aFc, aA, cz, n, bS, ct, aql, cd, ad, md, cc, MR, aEX, sN, Db, aRD, h, iY, Fx, K, lQ, AN, ae, af, dl, JU, cr, Z, QK, pb, wT, nM, ac, eM, mx, aS, y, cB, B); #c1(SRF) c2(NP_003122) c3(serum response factor isoform 1) c4(135471, 135472) c5(135473, 135474, 135475, 135476, 135477, 135478, 135479, 135480) c6( ) c7(T, aCh, AS, Am, ha, Xi, aFc, aA, cz, n, bS, ct, aql, cd, ad, md, cc, MR, aEX, sN, Db, aRD, h, iY, Fx, K, IQ, AN, ae, af, dl, JU, cr, Z, QK, pb, wT, nM, ac, eM, mx, aS, y, cB, B); #c1(SRFBP1) c2(NP_689759) c3(serum response factor-binding protein 1) c4(243888) c5(243889, 243890, 243891, 243892, 243893, 243894) c6( ) c7(iu, ci, T, cd, dx); #c1(SRGAP1) c2(NP_065813) c3(SLIT-ROBO Rho GTPase-activating protein 1) c4(205671) c5(205672, 205673, 205674, 205675, 205676, 205677) c6( ) c7(nA); #c1(SRGAP1) c2(XP_005269099) c3(SLIT-ROBO Rho GTPase-activating protein 1 isoform X1) c4(282299) c5(282300, 282301, 282302, 282303, 282304, 282305) c6( ) c7(nA); #c1(SRGAP2) c2(NP_001287881) c3(SLIT-ROBO Rho GTPase-activating protein 2 isoform d) c4(113118) c5(113119, 113120, 113121, 113122, 113123, 113124) c6( ) c7(kh, ZH, c, CN, bP, aW, Y, aT, Lb, nA); #c1(SRGAP2) c2(NP_056141) c3(SLIT-ROBO Rho GTPase-activating protein 2 isoform a) c4(185916, 185917, 185918) c5(185919, 185920, 185921, 185922, 185923, 185924, 185925, 185926, 185927, 185928, 185929, 185930) c6( ) c7(kh, ZH, c, CN, bP, aW, Y, aT, Lb, nA); #c1(SRGAP2) c2(XP_011507658) c3 (SLIT-ROBO Rho GTPase-activating protein 2 isoform X6) c4(297601, 297602) c5(297603) c6( ) c7(kh, ZH, c, CN, bP, aW, Y, aT, Lb, nA); #c1(SRGAP2) c2(XP_011507660) c3(SLIT-ROBO Rho GTPase-activating protein 2 isoform X7) c4( ) c5(297604, 297605, 297606, 297607, 297608, 297609, 297610, 297611, 297612, 297613, 297614) c6( ) c7(kh, ZH, c, CN, bP, aW, Y, aT, Lb, nA); #c1(SRGAP2) c2(XP_011507661) c3(SLIT-ROBO Rho GTPase-activating protein 2 isoform X8) c4( ) c5(297615, 297616, 297617, 297618, 297619, 297620) c6( ) c7(kh, ZH, c, CN, bP, aW, Y, aT, Lb, nA); #c1(SRGAP3) c2(NP_001028289) c3(SLIT-ROBO Rho GTPase-activating protein 3 isoform b) c4(30440) c5(30441, 30442, 30443, 30444, 30445, 30446) c6( ) c7(ZH, c, ac, CN, aW, aT, By, j, nA, an); #c1(SRGAP3) c2(NP_055665) c3(SLIT-ROBO Rho GTPase-activating protein 3 isoform a) c4(182620) c5(182621, 182622, 182623, 182624, 182625, 182626) c6( ) c7(ZH, c, ac, CN, aW, aT, By, j, nA, an); #c1(SRGAP3) c2(XP_011532602) c3(SLIT-ROBO Rho GTPase-activating protein 3 isoform X3) c4(328704) c5( ) c6( ) c7(ZH, c, ac, CN, aW, aT, By, j, nA, an); #c1(SRGAP3) c2(XP_011532604) c3(SLIT-ROBO Rho GTPase-activating protein 3 isoform X5) c4( ) c5(328705, 328706, 328707, 328708, 328709, 328710) c6( ) c7(ZH, c, ac, CN, aW, aT, By, j, nA, an); #c1(SRGN) c2(NP_002718) c3(serglycin precursor) c4(131392) c5(131393, 131394, 131395, 131396, 131397, 131398) c6( ) c7(T, dA, aE, aW, aT, it, fd, ey, K, qF, pz, jj, bt, KS); #c1(SRI) c2(NP_001243821) c3(sorcin isoform D) c4(91802) c5(91803, 91804, 91805, 91806, 91807, 91808) c6( ) c7(gV, T, afK, cd, eA, q, cc, xy, aT, K, rE, eG, hG, aW, bT, Pg, r, fq, ck, Mw, bt, iu, KG, qc); #c1(SRI) c2(NP_003121) c3(sorcin isoform A) c4(135470) c5( ) c6( ) c7(gV, T, afK, cd, eA, q, cc, xy, aT, K, rE, eG, hG, aW, bT, Pg, r, fq, ck, Mw, bt, iu, kG, qc); #c1(SRI) c2(NP_944490) c3(sorcin isoform B) c4(260622) c5(260623, 260624, 260625, 260626, 260627, 260628, 260629, 260630, 260631, 260632, 260633, 260634) c6( ) c7(gV, T, afK, cd, eA, q, cc, xy, aT, K, rE, eG, hG, aW, bT, Pg, r, fq, ck, Mw, bt, iu, kG, qc); #c1(SRL) c2(NP_001092284) c3(sarcalumenin precursor) c4(39515, 39516, 39517) c5(39518, 39519, 39520, 39521, 39522, 39523, 39524, 39525, 39526, 39527, 39528, 39529, 39530, 39531, 39532, 39533, 39534, 39535) c6( ) c7(bES, aj); #c1(SRMS) c2(NP_543013) c3(tyrosine-protein kinase Srms) c4(233251) c5(233252, 233253, 233254, 233255, 233256, 233257) c6( ) c7(aq, T, aT); #c1(SRP14) c2(NP_003125) c3(signal recognition particle 14 kDa protein) c4(135490) c5(135491, 135492, 135493, 135494, 135495, 135496) c6( ) c7(dx, Ea); #c1(SRP19) c2(NP_001191122) c3(signal recognition particle 19 kDa protein isoform 2) c4(81048) c5(81049, 81050, 81051, 81052, 81053, 81054) c6( ) c7(ac); #c1(SRP19) c2(NP_001191125) c3(signal recognition particle 19 kDa protein isoform 4) c4(81055) c5(81056, 81057, 81058, 81059, 81060) c6( ) c7(ac); #c1(SRP19) c2(NP_003126) c3(signal recognition particle 19 kDa protein isoform 1) c4(135497) c5(135498, 135499, 135500, 135501, 135502, 135503, 135504) c6( ) c7(ac); #c1(SRP68) c2(NP_001247431) c3(signal recognition particle subunit SRP68 isoform 2) c4(93581, 93582) c5(93583, 93584, 93585, 93586, 93587, 93588, 93589, 93590, 93591, 93592, 93593, 93594, 93595) c6( ) c7(Pk, Az, lQ); #c1(SRP68) c2(NP_001247432) c3(signal recognition particle subunit SRP68 isoform 3) c4( ) c5(93596, 93597, 93598, 93599, 93600) c6( ) c7(Pk, Az, lQ); #c1(SRP68) c2(NP_055045) c3(signal recognition particle subunit SRP68 isoform 1) c4(178529, 178530, 178531, 178532, 178533) c5(178534, 178535, 178536, 178537, 178538, 178539, 178540, 178541, 178542, 178543, 178544, 178545, 178546, 178547, 178548, 178549, 178550, 178551, 178552, 178553, 178554, 178555, 178556, 178557) c6( ) c7(Pk, Az, lQ); #c1(SRP72) c2(NP_001254651) c3(signal recognition particle subunit SRP72 isoform 2) c4(94790, 94791) c5(94792, 94793, 94794, 94795, 94796, 94797, 94798, 94799, 94800, 94801, 94802, 94803) c6( ) c7(bw, clV, br, bs); #c1(SRP72) c2(NP_008878) c3(signal recognition particle subunit SRP72 isoform 1) c4(169455) c5(169456, 169457, 169458, 169459, 169460, 169461) c6( ) c7(bw, clV, br, bs); #c1(SRP9) c2(NP_001123912) c3(signal recognition particle 9 kDa protein isoform 1) c4(47984) c5(47985, 47986, 47987, 47988, 47989, 47990, 47991, 47992, 47993, 47994, 47995) c6( ) c7(X); #c1(SRP9) c2(NP_003124) c3(signal recognition particle 9 kDa protein isoform 2) c4(135481, 135482) c5(135483, 135484, 135485, 135486, 135487, 135488, 135489) c6( ) c7(X); #c1(SRPK1) c2(NP_003128) c3(SRSF protein kinase 1) c4(135505, 135506) c5(135507, 135508, 135509, 135510, 135511, 135512, 135513, 135514, 135515, 135516, 135517, 135518, 135519, 135520) c6( ) c7(iu, T, aC, iM, Dy, fR, s, vT, aW, Y, Qu, X, eM, aT, lo, je, m, t, q, cp, hX); #c1(SRPK2) c2(NP_872633) c3(SRSF protein kinase 2 isoform b precursor) c4(256591) c5(256592, 256593, 256594, 256595, 256596) c6( ) c7(ck, iu, fR, eM, Qu, lo, lR, bt, fq, pb, hX); #c1(SRPK2) c2(NP_872634) c3(SRSF protein kinase 2 isoform a) c4(256597) c5( ) c6( ) c7(ck, iu, fR, eM, Qu, lo, lR, bt, fq, pb, hX); #c1(SRPK2) c2(XP_011514836) c3(SRSF protein kinase 2 isoform X4) c4( ) c5(307566, 307567, 307568, 307569, 307570, 307571, 307572, 307573, 307574, 307575, 307576, 307577, 307578, 307579, 307580, 307581) c6( ) c7(ck, iu, fR, eM, Qu, lo, lR, bt, fq, pb, hX); #c1(SRPK2) c2(XP_011514840) c3(SRSF protein kinase 2 isoform X2) c4(307582) c5( ) c6( ) c7(ck, iu, fR, eM, Qu, lo, lR, bt, fq, pb, hX); #c1(SRPK2) c2(XP_011514841) c3(SRSF protein kinase 2 isoform X10) c4( ) c5(307583) c6( ) c7(ck, iu, fR, eM, Qu, lo, lR, bt, fq, pb, hX); #c1(SRPR) c2(NP_001171313) c3(signal recognition particle receptor subunit alpha isoform 2) c4(70041) c5(70042, 70043, 70044, 70045, 70046, 70047) c6( ) c7(es, Us); #c1(SRPR) c2(NP_003130) c3(signal recognition particle receptor subunit alpha isoform 1) c4(135521) c5(135522, 135523, 135524, 135525, 135526, 135527) c6( ) c7(es, Us); #c1(SRPRB) c2(NP_067026) c3(signal recognition particle receptor subunit beta) c4(208503, 208504) c5(208505, 208506, 208507, 208508, 208509, 208510, 208511, 208512, 208513, 208514, 208515, 208516) c6( ) c7(aW, X, B, eA); #c1(SRPX) c2(NP_001164221) c3(sushi repeat-containing protein SRPX isoform 2 precursor) c4(66822) c5(66823, 66824, 66825, 66826, 66827, 66828, 66829, 66830, 66831, 66832, 66833, 66834) c6( ) c7(T, bS, ey, ci, ct, g, Z, nC, hq); #c1(SRPX) c2(NP_001164222) c3(sushi repeat-containing protein SRPX isoform 3 precursor) c4(66835) c5(66836, 66837, 66838, 66839, 66840, 66841) c6( ) c7(T, bS, ey, ci, ct, g, Z, nC, hq); #c1(SRPX) c2(NP_001164223) c3(sushi repeat-containing protein SRPX isoform 4 precursor) c4(66842) c5(66843, 66844, 66845, 66846, 66847, 66848) c6( ) c7(T, bS, ey, ci, ct, g, Z, nC, hq); #c1(SRPX) c2(NP_006298) c3(sushi repeat-containing protein SRPX isoform 1 precursor) c4(163802, 163803) c5(163804, 163805, 163806, 163807, 163808, 163809) c6( ) c7(T, bS, ey, ci, ct, g, Z, nC, hq); #c1(SRPX2) c2(XP_005262178) c3(sushi repeat-containing protein SRPX2 isoform X1) c4(277767) c5(277768, 277769, 277770, 277771, 277772, 277773) c6( ) c7(T, bwZ, hF, oe, ha, gX, r, VT, aA, akf, aF, nA, KU, g, clW, q); #c1(SRR) c2(XP_011522276) c3(serine racemase isoform X1) c4(316947) c5(316948, 316949, 316950, 316951, 316952, 316953) c6( ) c7(bL, dP, bM, c, wD, e, eZ, aj, DG, fD, J, ek); #c1(SRRM1) c2(NP_001290377) c3(serine/arginine repetitive matrix protein 1 isoform 1) c4(115324) c5( ) c6( ) c7(aPF); #c1(SRRM1) c2(XP_011538784) c3(serine/arginine repetitive matrix protein 1 isoform X8) c4( ) c5(336518, 336519) c6( ) c7(aPF); #c1(SRRM1) c2(XP_011538785) c3(serine/arginine repetitive matrix protein 1 isoform X9) c4(336520) c5(336521, 336522, 336523, 336524, 336525, 336526, 336527, 336528, 336529, 336530, 336531, 336532, 336533, 336534, 336535, 336536) c6( ) c7(aPF); #c1(SRRM2) c2(NP_057417) c3(serine/arginine repetitive matrix protein 2) c4(190677) c5(190678, 190679, 190680, 190681, 190682, 190683, 190684, 190685, 190686, 190687, 190688, 190689, 190690, 190691, 190692, 190693, 190694, 190695, 190696) c6( ) c7(bM, T, aC, ha, jh, aA, bB, tc, TM, Vd, ci, nA, lR, fr, hF, eA, aR, ad, hX, eW, ft, aE, WY, ds, SV, aT, kf, Ak, dV, h, K, an, brV, fb, ae, af, dx, il, d, vT, aW, Y, ff, eZ, nf, aq, aaE, aF, Ku, en, iR, bz, pb, QA, bN, dR, ck, rR, kP, c, eM, bE, Qu, dh, mx, U, aEs, rx, xh, arg, B); #c1(SRRM2) c2(XP_005255283) c3(serine/arginine repetitive matrix protein 2 isoform X1) c4(273582, 273583) c5(273584) c6( ) c7(bM, T, aC, ha, jh, aA, bB, tc, TM, Vd, ci, nA, lR, fr, hF, eA, aR, ad, hX, eW, ft, aE, WY, ds, SV, aT, kf, Ak, dV, h, K, an, brV, fb, ae, af, dx, iL, d, vT, aW, Y, ff, eZ, nf, aq, aaE, aF, Ku, en, iR, bz, pb, QA, bN, dR, ck, rR, kP, c, eM, bE, Qu, dh, mx, U, aEs, rx, xh, arg, B); #c1(SRRM2) c2(XP_005255284) c3(serine/arginine repetitive matrix protein 2 isoform X2) c4(273585, 273586, 273587) c5(273588, 273589, 273590, 273591, 273592, 273593, 273594, 273595, 273596, 273597, 273598, 273599, 273600, 273601, 273602, 273603, 273604, 273605, 273606) c6( ) c7(bM, T, aC, ha, jh, aA, bB, tc, TM, Vd, ci, nA, lR, fr, hF, eA, aR, ad, hX, eW, ft, aE, WY, ds, SV, aT, kf, Ak, dV, h, K, an, brV, fb, ae, af, dx, iL, d, vT, aW, Y, ff, eZ, nf, aq, aaE, aF, Ku, en, iR, bz, pb, QA, bN, dR, ck, rR, kP, c, eM, bE, Qu, dh, mx, U, aEs, rx, xh, arg, B); #c1 (SRRM2) c2(XP_011520745) c3(serine/arginine repetitive matrix protein 2 isoform X4) c4( ) c5(315762) c6( ) c7(bM, T, aC, ha, jh, aA, bB, tc, TM, Vd, ci, nA, lR, fr, hF, eA, aR, ad, hX, eW, ft, aE, WY, ds, SV, aT, kf, Ak, dV, h, K, an, brV, fb, ae, af, dx, il, d, vT, aW, Y, ff, eZ, nf, aq, aaE, af, Ku, en, iR, bz, pb, QA, bN, dR, ck, rR, kP, c, eM, bE, Qu, dh, mx, U, aEs, rx, xh, arg, B); #c1(SRRM4) c2(NP_919262) c3(serine/arginine repetitive matrix protein 4) c4(258347) c5(258348, 258349, 258350, 258351, 258352, 258353, 258354) c6( ) c7(ft); #c1(SRRM4) c2(XP_011537157) c3(serine/arginine repetitive matrix protein 4 isoform X1) c4( ) c5(334917, 334918, 334919) c6( ) c7(ft); #c1(SRRT) c2(XP_005250462) c3(serrate RNA effector molecule homolog isoform X1) c4(270323) c5(270324, 270325, 270326, 270327, 270328, 270329) c6( ) c7(jz, bO); #c1(SRRT) c2(XP_005250463) c3(serrate RNA effector molecule homolog isoform X2) c4(270330, 270331) c5(270332, 270333, 270334, 270335, 270336, 270337, 270338, 270339) c6( ) c7(jz, bO); #c1(SRRT) c2(XP_005250464) c3(serrate RNA effector molecule homolog isoform X3) c4(270340) c5(270341, 270342, 270343, 270344, 270345, 270346) c6( ) c7(jz, bO); #c1(SRRT) c2(XP_005250465) c3(serrate RNA effector molecule homolog isoform X4) c4(270347, 270348) c5(270349, 270350, 270351, 270352, 270353, 270354) c6( ) c7(jz, bO); #c1(SRRT) c2(XP_006716086) c3(serrate RNA effector molecule homolog isoform X6) c4( ) c5(290025, 290026, 290027, 290028, 290029) c6( ) c7(jz, bO); #c1(SRRT) c2(XP_011514593) c3(serrate RNA effector molecule homolog isoform X5) c4( ) c5(307301, 307302, 307303, 307304, 307305) c6( ) c7(jz, bO); #c1(SRSF1) c2(NP_001071634) c3(serine/arginine-rich splicing factor 1 isoform 2) c4(35353) c5(35354, 35355, 35356, 35357, 35358, 35359) c6( ) c7(T, hF, q, av, lg, ds, rC, aT, kf, h, on, cp, il, aW, ff, ahk, lb, eZ, r, aq, lo, en, iR, cm, bz, ck, es, ac, eM, iA, Qu, iu); #c1(SRSF1) c2(NP_008855) c3(serine/arginine-rich splicing factor 1 isoform 1) c4(169138) c5(169139, 169140, 169141, 169142, 169143, 169144) c6( ) c7(T, hF, q, av, lg, ds, rC, aT, kf, h, on, cp, il, aW, ff, ahk, lb, eL, r, aq, lo, en, iR, cm, bz, ck, es, ac, eM, iA, Qu, iu); #c1(SRSF10) c2(NP_001287865) c3(serine/arginine-rich splicing factor 10 isoform 8) c4(113095) c5(113096, 113097, 113098, 113099, 113100, 113101) c6( ) c7(t, kl, hL); #c1(SRSF11) c2(XP_011540729) c3(serine/arginine-rich splicing factor 11 isoform X1) c4(339290, 339291) c5( ) c6( ) c7(q, ci); #c1(SRSF11) c2(XP_011540733) c3(serine/arginine-rich splicing factor 11 isoform X4) c4( ) c5(339292, 339293, 339294, 339295, 339296, 339297) c6( ) c7(q, ci); #c1(SRSF11) c2(XP_011540735) c3(serine/arginine-rich splicing factor 11 isoform X7) c4( ) c5(339298) c6( ) c7(q, ci); #c1(SRSF12) c2(NP_542781) c3(serine/arginine-rich splicing factor 12) c4(233056) c5(233057, 233058, 233059, 233060, 233061, 233062) c6( ) c7(af); #c1(SRSF2) c2(NP_001182356) c3(serine/arginine-rich splicing factor 2) c4(74824, 74825, 74826, 74827, 74828) c5(74829, 74830, 74831, 74832, 74833, 74834, 74835, 74836, 74837, 74838, 74839, 74840, 74841, 74842, 74843, 74844, 74845, 74846, 74847, 74848, 74849, 74850, 74851, 74852, 74853, 74854, 74855, 74856, 74857, 74858) c6( ) c7(T, atk, jh, bs, ol, ci, nA, zP, br, x, av, Hx, b, iu, h, zp, on, pq, ajU, zl, xX, ahk, bw, bz, yR, dR, ck, c, SE, Qu, eM, wl, pg, i, bt, B); #c1(SRSF3) c2(NP_003008) c3(serine/arginine-rich splicing factor 3) c4(134393, 134394) c5(134395, 134396, 134397, 134398, 134399, 134400, 134401, 134402, 134403, 134404, 134405, 134406) c6( ) c7(T, pZ, bE, aqJ, ds, EQ, bB, cm, bK, m, bNL, hf, t, dr); #c1(SRSF4) c2(XP_011540253) c3(serine/arginine-rich splicing factor 4 isoform X1) c4( ) c5(338655, 338656, 338657, 338658, 338659, 338660, 338661, 338662, 338663, 338664, 338665, 338666, 338667, 338668, 338669, 338670, 338671, 338672, 338673) c6( ) c7(pt, dF, B, vi); #c1(SRSF5) c2(XP_005268055) c3(serine/arginine-rich splicing factor 5 isoform X1) c4(281427) c5( ) c6( ) c7(T, uk, Ew, iq, pZ, bE, aW, Ls, ahk, hl, lb, aT, ex, eV, ajM, bS, ct, Z, ae, HO, ad); #c1(SRSF5) c2(XP_005268057) c3(serine/arginine-rich splicing factor 5 isoform X2) c4(281428) c5(281429, 281430, 281431, 281432, 281433, 281434, 281435, 281436, 281437, 281438, 281439, 281440, 281441, 281442, 281443, 281444, 281445, 281446, 281447, 281448) c6( ) c7(T, uk, Ew, iq, pZ, bE, aW, Ls, ahk, hl, lb, aT, ex, eV, ajM, bS, ct, Z, ae, HO, ad); #c1(SRSF6) c2(NP_006266) c3(serine/arginine-rich splicing factor 6) c4(163436) c5(163437, 163438, 163439, 163440, 163441, 163442) c6( ) c7(T, bE, yk, ahk, rC, aT, bB, pt); #c1(SRSF7) c2(NP_001026854) c3(serine/arginine-rich splicing factor 7 isoform 1) c4(29661) c5( ) c6( ) c7(U); #c1(SRSF7) c2(NP_001182375) c3(serine/arginine-rich splicing factor 7 isoform 2) c4(74874) c5( ) c6( ) c7(U); #c1(SRSF7) c2(XP_005264542) c3(serine/arginine-rich splicing factor 7 isoform X2) c4( ) c5(279004, 279005, 279006, 279007, 279008, 279009, 279010, 279011, 279012, 279013, 279014, 279015) c6( ) c7(U); #c1(SRSF9) c2(NP_003760) c3(serine/arginine-rich splicing factor 9) c4(140657) c5(140658, 140659, 140660, 140661, 140662, 140663) c6( ) c7(h, pt, bb, pZ); #c1(SRXN1) c2(NP_542763) c3(sulfiredoxin-1) c4(233022) c5(233023, 233024, 233025, 233026, 233027, 233028) c6( ) c7(aW, fr, K, aT); #c1(SRY) c2(NP_003131) c3(sex-determining region Y protein) c4(135528, 135529, 135530) c5(135531, 135532, 135533, 135534, 135535, 135536, 135537, 135538, 135539, 135540, 135541, 135542, 135543, 135544, 135545, 135546, 135547, 135548, 135549) c6( ) c7(bfh, T, gU, Ke, acz, aec, cz, adW, n, aj, v, clX, eb, bjN, Fb, lR, Ul, clY, q, J, ad, KL, hX, er, Bl, ft, aeZ, aE, clZ, vJ, Pl, MP, aT, FN, bff, vH, UD, rO, PD, h, brV, ae, clk, UF, aeT, UL, ND, bgA, bfg, aHW, aeC, aek, xs, Y, aW, r, pr, aGb, ff, Pp, Ku, en, cm, pb, Di, kc, Uu, aeB, Qu, afc, eB, uo, Ux, Uw, Le, KY, B, y); #c1(SS18) c2(NP_001007560) c3(protein SSXT isoform 1) c4(22795, 22796, 22797, 22798, 22799) c5(22800, 22801, 22802, 22803, 22804, 22805, 22806, 22807, 22808, 22809, 22810, 22811) c6( ) c7(jf, bv, T, blC, aNA, Qf, QG, n, cJa, hP, bu, iJ, dx); #c1(SS18) c2(NP_005628) c3(protein SSXT isoform 2) c4(158080, 158081) c5(158082, 158083, 158084, 158085, 158086, 158087, 158088, 158089) c6( ) c7(jf, bv, T, blC, aNA, Qf, QG, n, cJa, hP, bu, iJ, dx); #c1(SS18) c2(XP_006722590) c3(protein SSXT isoform X3) c4( ) c5(294757, 294758, 294759, 294760, 294761, 294762, 294763, 294764, 294765, 294766, 294767) c6( ) c7(jf, bv, T, blC, aNA, Qf, QG, n, cJa, hP, bu, iJ, dx); #c1(SS18) c2(XP_011524454) c3(protein SSXT isoform X4) c4( ) c5(319041, 319042, 319043, 319044, 319045, 319046, 319047, 319048) c6( ) c7(jf, bv, T, blC, aNA, Qf, QG, n, cJa, hP, bu, iJ, dx); #c1(SS18L1) c2(NP_945173) c3(calcium-responsive transactivator isoform 1) c4(260688) c5(260689, 260690, 260691, 260692, 260693, 260694, 260695, 260696, 260697, 260698) c6( ) c7(pb, Ck, ac, Y, eZ, dy, lS, lR, bc, lC, iJ, bDY, lx); #c1(SS18L1) c2(XP_011527068) c3 (calcium-responsive transactivator isoform X2) c4( ) c5(321507) c6( ) c7(pb, Ck, ac, Y, eZ, dy, lS, lR, bc, lC, iJ, bDY, lx); #c1(SS18L1) c2(XP_011527069) c3(calcium-responsive transactivator isoform X3) c4(321508, 321509) c5(321510, 321511, 321512, 321513, 321514, 321515, 321516, 321517, 321518, 321519, 321520, 321521) c6( ) c7(pb, Ck, ac, Y, eZ, dy, lS, lR, bc, lC, iJ, bDY, lx); #c1(SSB) c2(NP_003133) c3 (lupus La protein) c4(135565, 135566, 135567, 135568, 135569, 135570) c5(135571, 135572, 135573, 135574, 135575, 135576, 135577, 135578, 135579, 135580, 135581, 135582, 135583, 135584, 135585, 135586, 135587, 135588, 135589, 135590, 135591, 135592, 135593, 135594, 135595, 135596, 135597, 135598, 135599, 135600, 135601, 135602, 135603, 135604, 135605, 135606, 135607, 135608, 135609, 135610) c6( ) c7(Ob, gV, T, aFQ, jx, fN, ai, mC, hX, xy, rw, hD, lQ, dl, bc, bnX, hG, eZ, pb, uw, AH, dy, fl, y); #c1(SSBP1) c2(NP_001243442) c3(single-stranded DNA-binding protein, mitochondrial precursor) c4(90770, 90771, 90772, 90773) c5(90774, 90775, 90776, 90777, 90778, 90779, 90780, 90781, 90782, 90783, 90784, 90785, 90786, 90787, 90788, 90789, 90790, 90791, 90792, 90793, 90794, 90795, 90796, 90797, 90798, 90799, 90800, 90801, 90802, 90803, 90804, 90805, 90806, 90807, 90808, 90809, 90810, 90811, 90812, 90813, 90814, 90815, 90816, 90817, 90818, 90819, 90820, 90821, 90822, 90823, 90824, 90825) c6( ) c7(wr, Pk, xH, lQ, cm, eW); #c1(SSBP2) c2(NP_001243662) c3 (single-stranded DNA-binding protein 2 isoform 3) c4(91321) c5(91322, 91323, 91324, 91325, 91326, 91327) c6( ) c7(ck, x, T, d, iT, iX, it, k, bt, ae, ad, sj); #c1(SSBP2) c2(NP_036578) c3(single-stranded DNA-binding protein 2 isoform 2) c4(174478, 174479) c5(174480, 174481, 174482, 174483, 174484, 174485, 174486, 174487, 174488, 174489, 174490, 174491) c6( ) c7(ck, x, T, d, iT, iX, it, k, bt, ae, ad, sj); #c1(SSFA2) c2(NP_001123917) c3(sperm-specific antigen 2 isoform 1) c4(48011) c5(48012, 48013, 48014, 48015, 48016, 48017) c6( ) c7(bB, bE, T, fd); #c1(SSFA2) c2(NP_001274432) c3(sperm-specific antigen 2 isoform 3) c4(106534) c5(106535) c6( ) c7(bB, bE, T, fd); #c1(SSFA2) c2(NP_001274433) c3(sperm-specific antigen 2 isoform 4) c4( ) c5(106536, 106537, 106538, 106539, 106540) c6( ) c7(bB, bE, T, fd); #c1(SSFA2) c2(NP_006742) c3(sperm-specific antigen 2 isoform 2) c4(167626) c5(167627, 167628, 167629, 167630, 167631, 167632) c6( ) c7(bB, bE, T, fd); #c1(SSH1) c2(NP_001154802) c3(protein phosphatase Slingshot homolog 1 isoform 2) c4(61080) c5(61081, 61082, 61083, 61084, 61085, 61086, 61087, 61088, 61089, 61090, 61091, 61092) c6( ) c7(eW, aQo, agH, aA); #c1(SSH1) c2(NP_001154803) c3(protein phosphatase Slingshot homolog 1 isoform 3) c4(61093) c5(61094, 61095, 61096, 61097, 61098, 61099, 61100) c6( ) c7(eW, aQo, agH, aA); #c1(SSH1) c2(NP_061857) c3(protein phosphatase Slingshot homolog 1 isoform 1) c4(200832, 200833) c5(200834, 200835, 200836, 200837, 200838, 200839) c6( ) c7(eW, aQo, agH, aA); #c1(SSH1) c2(XP_005269043) c3(protein phosphatase Slingshot homolog 1 isoform X7) c4(282261) c5(282262, 282263, 282264, 282265, 282266, 282267) c6( ) c7(eW, aQo, agH, aA); #c1(SSH2) c2(NP_001269058) c3(protein phosphatase Slingshot homolog 2 isoform 1) c4(100343) c5(100344, 100345, 100346, 100347, 100348, 100349) c6( ) c7(buL, hd, fr, ci, aA); #c1(SSH2) c2(NP_001269060) c3(protein phosphatase Slingshot homolog 2 isoform 4) c4(100350) c5(100351, 100352, 100353, 100354, 100355, 100356) c6( ) c7(buL, hd, fr, ci, aA); #c1(SSH2) c2(NP_203747) c3 (protein phosphatase Slingshot homolog 2 isoform 2) c4(228475, 228476) c5(228477, 228478, 228479, 228480, 228481, 228482, 228483, 228484, 228485, 228486, 228487, 228488) c6( ) c7(buL, hd, fr, ci, aA); #c1(SSMEM1) c2(NP_660311) c3(serine-rich single-pass membrane protein 1) c4(240871) c5(240872, 240873, 240874, 240875, 240876, 240877) c6( ) c7(cd); #c1(SSNA1) c2(NP_003722) c3(Sjoegren syndrome nuclear autoantigen 1) c4(140444) c5(140445, 140446, 140447, 140448, 140449, 140450) c6( ) c7(pb, aFQ); #c1(SSPN) c2(NP_005077) c3(sarcospan isoform 1) c4(152666, 152667, 152668, 152669, 152670) c5(152671, 152672, 152673, 152674, 152675, 152676, 152677, 152678, 152679, 152680, 152681, 152682, 152683, 152684, 152685, 152686, 152687, 152688, 152689, 152690) c6( ) c7(cc, aD, xs, km, wR, fr, bgi); #c1(SSPN) c2(XP_011519157) c3(sarcospan isoform X1) c4( ) c5(313756, 313757, 313758, 313759, 313760, 313761, 313762, 313763, 313764, 313765, 313766, 313767, 313768, 313769) c6( ) c7(cc, aD, xs, km, wR, fr, bgi); #c1(SSPO) c2(NP_940857) c3(SCO-spondin precursor) c4( ) c5(259960, 259961, 259962, 259963, 259964, 259965, 259966, 259967, 259968, 259969, 259970, 259971, 259972, 259973, 259974, 259975, 259976) c6( ) c7(c); #c1(SSR1) c2(NP_003135) c3(translocon-associated protein subunit alpha isoform 1 precursor) c4(135611, 135612) c5(135613, 135614, 135615, 135616, 135617, 135618, 135619, 135620, 135621, 135622, 135623, 135624) c6( ) c7(T, avm, aW, ff, ht, eH, aT, aeV, ey, en, hu, eF); #c1(SSR2) c2(XP_011508225) c3(translocon-associated protein subunit beta isoform X2) c4(298858, 298859, 298860) c5(298861, 298862) c6( ) c7(cm, pX, ad); #c1(SSR2) c2(XP_011508226) c3(translocon-associated protein subunit beta isoform X1) c4( ) c5(298863, 298864, 298865, 298866, 298867, 298868, 298869, 298870, 298871, 298872, 298873, 298874, 298875, 298876, 298877, 298878, 298879, 298880, 298881, 298882) c6( ) c7(cm, pX, ad); #c1(SSRP1) c2(NP_003137) c3(FACT complex subunit SSRP1) c4(135625) c5(135626, 135627, 135628, 135629, 135630, 135631, 135632) c6( ) c7(ar, T, y, vJ, aW, eZ, al, nm, lo, wb, fl, zC, C, aGj, KY, ae, ad); #c1(SSSCA1) c2(NP_006387) c3(Sjoegren syndrome/scleroderma autoantigen 1 isoform 1) c4(164567) c5(164568, 164569, 164570, 164571, 164572, 164573, 164574, 164575) c6(164576, 164577) c7(T, ea, if, fd, bB, ci, aE, il, Qk, K, eL, bc, xQ, yo, aW, as, cm, bw, GR, eM, nB, dc, go, vA, cj, m, dD, NT, n, fr, qN, jz, h, dL, ge, aq, zZ, iO, bP, hR, it, B, fM, aoE, aA, q, eA, XX, aT, dz, ae, af, jf, s, iU, Y, r, en, cl, t, ik, pb, aEp, ck, nM, iM, dy, bt, On, hS, hl, Ke, pY, jh, eq, ad, hX, hd, ib, fb, d, je, et, ff, apb, aF, bz, bE, aS, EB); #c1(SST) c2(NP_001039) c3(somatostatin preproprotein) c4(34207) c5( ) c6( ) c7(T, aC, Tb, clb, bO, fd, bB, ci, Jx, ip, Vp, aE, pX, aeO, yo, aeR, Jl, aW, Ls, az, LZ, g, as, cm, eM, nB, vA, vy, bmt, uJ, m, bBY, hh, gD, OF, xn, lT, wu, ht, aj, fr, ml, JW, eW, ke, ft, Do, h, jy, gl, vx, cly, bmu, ks, aq, Hu, mb, ar, bkn, W, cA, bpC, dB, Qu, hL, hR, anx, cB, B, bL, iH, jx, aA, cz, hy, C, il, pd, q, J, eA, mj, mP, aAF, nv, hb, aT, bK, ae, af, dl, CB, apL, jf, Uf, s, nO, Y, apk, r, en, cl, wA, t, iK, acp, bN, Ta, bNx, iW, jh, dG, oH, fu, OH, Fc, bfD, ad, dP, av, Tg, hd, iC, io, hD, Ti, hP, wm, fb, Yi, d, BN, lR, ff, aF, iV, bz, bF, OG, VO, mi, bE, AR, ra, y, xg, ne, Tc); #c1(SSTR1) c2(NP_001040) c3(somatostatin receptor type 1) c4(34224) c5( ) c6( ) c7(aC, lT, ha, aA, hy, eA, ad, mj, aE, mP, Do, pX, aT, bq, fb, ae, Yi, yo, BN, s, aW, ks, aF, nB, hR, cB, ne); #c1(SSTR2) c2(NP_001041) c3(somatostatin receptor type 2) c4( ) c5( ) c6( ) c7(T, dD, aC, AS, lT, vf, ea, bXJ, ha, bB, hz, hy, v, eq, ci, il, JW, eA, ad, eJ, ip, av, tg, ft, mj, aE, mP, Do, nv, Db, pX, aT, vg, hD, blg, Nf, AN, bq, fb, ae, Yi, jf, yo, B, BN, s, aW, ks, EZ, g, cm, OG, eM, nB, cj, hR, bmt, cB, ne); #c1(SSTR3) c2(XP_005261778) c3(somatostatin receptor type 3 isoform X1) c4(277605) c5(277606, 277607, 277608, 277609, 277610, 277611, 277612, 277613, 277614, 277615) c6( ) c7(T, aC, akd, fR, aA, ad, mj, hd, pX, aT, dL, bq, ae, yo, s, Jl, vj, aF, g, OG, nB, at, cB); #c1(SSTR4) c2(NP_001043) c3(somatostatin receptor type 4) c4(34342) c5(34343, 34344, 34345, 34346, 34347, 34348) c6( ) c7(bL, bM, T, iH, yk, hU, ha, cz, fd, v, ci, yp, ye, xY, cd, ad, eA, yn, av, mj, xW, aT, h, xV, Fx, K, yb, gl, bK, yh, yj, ae, yi, xX, d, xU, s, n, aW, Y, yc, ahJ, yq, g, t, nC, bz, ik, iP, dR, jq, ok, yf, bP, yl, xZ, cj, tl, m, y, cB); #c1(SSTR5) c2(NP_001166031) c3(somatostatin receptor type 5) c4(68078, 68079) c5(68080, 68081, 68082, 68083, 68084, 68085, 68086, 68087, 68088, 68089, 68090, 68091, 68092, 68093, 68094, 68095) c6( ) c7(T, aC, lT, vf, mS, ci, OH, JW, ad, tg, mj, aE, Do, mW, cJc, aT, vg, blg, bK, bq, mY, he, mV, ae, mU, yo, s, ahJ, g, mZ, bN, mX, OG, cA, AR, bmt, hR, cB, mT); #c1(SSUH2) c2(NP_001243677) c3 (protein SSUH2 homolog isoform 1) c4(91346) c5(91347, 91348, 91349, 91350, 91351, 91352, 91353, 91354) c6( ) c7(iP); #c1(SSUH2) c2(NP_057015) c3(protein SSUH2 homolog isoform 3) c4(188515, 188516) c5(188517, 188518, 188519, 188520, 188521, 188522, 188523, 188524, 188525, 188526, 188527, 188528, 188529, 188530, 188531, 188532) c6( ) c7(iP); #c1(SSUH2) c2(XP_011532076) c3(protein SSUH2 homolog isoform X1) c4(328133) c5(328134, 328135, 328136, 328137) c6( ) c7(iP); #c1(SSUH2) c2(XP_011532077) c3(protein SSUH2 homolog isoform X2) c4( ) c5(328138, 328139) c6( ) c7(iP); #c1(SSX1) c2(NP_005626) c3(protein SSX1) c4(158062, 158063) c5(158064, 158065, 158066, 158067, 158068, 158069, 158070, 158071, 158072, 158073, 158074, 158075, 158076, 158077, 158078, 158079) c6( ) c7(ck, T, blC, Qf, af, Y, fd, bX, bu, iJ, B); #c1(SSX2B) c2(NP_001157889) c3(protein SSX2 isoform b) c4(62865, 62866) c5(62867, 62868, 62869, 62870, 62871, 62872, 62873, 62874, 62875, 62876, 62877, 62878, 62879, 62880, 62881) c6( ) c7(T, aNA, wu, ht, jh, fd, bB, iJ, aHy, ad, bv, aT, hP, K, wm, hu, ae, aW, Y, eH, r, bu, eF, blC, QG, Qf, iT, cJa, bt); #c1(SSX2B) c2(NP_001265630) c3(protein SSX2 isoform a) c4(99057) c5(99058, 99059, 99060, 99061, 99062, 99063, 99064, 99065, 99066, 99067, 99068, 99069) c6( ) c7(T, aNA, wu, ht, jh, fd, bB, iJ, aHy, ad, bv, aT, hP, K, wm, hu, ae, aW, Y, eH, r, bu, eF, blC, QG, Qf, iT, cJa, bt); #c1(SSX2IP) c2(XP_011538914) c3(afadin- and alpha-actinin-binding protein isoform X4) c4(336975, 336976) c5(336977, 336978, 336979, 336980, 336981, 336982, 336983, 336984, 336985, 336986, 336987, 336988) c6( ) c7(ey, j); #c1(SSX4B) c2(NP_001030004) c3(protein SSX4 isoform a) c4(30805) c5(30806, 30807, 30808, 30809, 30810, 30811, 30812, 30813, 30814, 30815, 30816) c6( ) c7(T, dB, Y, fd, bX, bu, iJ); #c1(SSX4B) c2(NP_001035702) c3(protein SSX4 isoform b) c4(32959) c5(32960, 32961, 32962, 32963, 32964, 32965, 32966, 32967, 32968, 32969) c6( ) c7(T, dB, Y, fd, bX, bu, iJ); #c1(SSX5) c2(NP_066295) c3(protein SSX5 isoform a) c4(207(22) c5(207123, 207124, 207125, 207126, 207127, 207128, 207129) c6( ) c7(T, B, Y); #c1(SSX5) c2(XP_011542251) c3 (protein SSX5 isoform X1) c4(340766, 340767) c5(340768, 340769, 340770, 340771, 340772, 340773, 340774, 340775, 340776, 340777, 340778, 340779, 340780, 340781, 340782, 340783, 340784, 340785, 340786) c6( ) c7(T, B, Y); #c1(ST13) c2(NP_003923) c3(hsc70-interacting protein isoform 1) c4(142184, 142185, 142186, 142187) c5(142188, 142189, 142190, 142191, 142192, 142193, 142194, 142195, 142196, 142197, 142198, 142199, 142200, 142201, 142202, 142203, 142204, 142205, 142206, 142207, 142208, 142209, 142210, 142211) c6( ) c7(bL, T, id, it, bB, aj, v, q, J, ad, eA, ft, anl, ds, bl, K, jj, aVV, zy, ae, d, Y, ff, CT, r, aq, en, g, bz, bE, cj, kS, B); #c1(ST14) c2(NP_068813) c3(suppressor of tumorigenicity 14 protein) c4(210785) c5(210786, 210787, 210788, 210789, 210790, 210791) c6( ) c7(T, dD, ea, sM, bB, eq, ci, nA, qN, fr, cJd, eA, ad, ds, qh, gd, aT, oj, fn, ji, ae, dF, sb, aW, ev, ff, xs, hl, fe, aq, aEW, Ku, Pd, en, t, g, bz, iV, alu, aUJ, bE, wE, gX, Fw, m, bD, vi); #c1(ST18) c2(NP_055497) c3(suppression of tumorigenicity 18 protein) c4(181548, 181549) c5(181550, 181551, 181552, 181553, 181554, 181555, 181556, 181557, 181558, 181559, 181560, 181561, 181562, 181563, 181564, 181565, 181566, 181567) c6( ) c7(aW, WP, aT, sa, iu, lJ); #c1(ST20) c2(NP_001094349) c3(suppressor of tumorigenicity 20 protein) c4(40687) c5(40688, 40689, 40690, 40691, 40692, 40693, 40694, 40695, 40696, 40697) c6( ) c7(T, if, r, aq, q, qN, iR, B); #c1(ST3GAL1) c2(XP_005251082) c3(CMP-N-acetylneuraminate-beta-galactosamide-alpha-2,3-sialyltransferase 1 isoform X1) c4(270750, 270751, 270752, 270753) c5(270754, 270755, 270756, 270757, 270758, 270759, 270760, 270761, 270762, 270763, 270764, 270765, 270766, 270767, 270768, 270769, 270770, 270771, 270772, 270773, 270774, 270775, 270776, 270777, 270778, 270779, 270780, 270781, 270782, 270783, 270784, 270785, 270786, 270787, 270788, 270789, 270790, 270791, 270792, 270793, 270794, 270795, 270796, 270797, 270798, 270799, 270800) c6(270801, 270802, 270803) c7(T, oe, n, ci, ade, cd, ad, dY, aT, hD, dL, bK, eL, af, ae, sj, aW, gl, agF, t, pb, ac, m, cn); #c1(ST3GAL2) c2(NP_008858) c3(CMP-N-acetylneuraminate-beta-galactosamide-alpha-2,3-sialyltransferase 2) c4(169145) c5(169146, 169147, 169148, 169149, 169150, 169151, 169152, 169153, 169154, 169155, 169156, 169157, 169158, 169159) c6(169160, 169161, 169162, 169163) c7(ae, ad, ati); #c1(ST3GAL4) c2(NP_001241687) c3(CMP-N-acetylneuraminate-beta-galactosamide-alpha-2,3-sialyltransferase 4 isoform 2) c4(89055, 89056) c5(89057, 89058, 89059, 89060, 89061, 89062, 89063, 89064, 89065, 89066, 89067, 89068, 89069, 89070, 89071, 89072, 89073, 89074, 89075, 89076, 89077, 89078, 89079, 89080, 89081, 89082, 89083, 89084, 89085, 89086, 89087, 89088, 89089, 89090) c6(89091, 89092, 89093, 89094, 89095, 89096, 89097, 89098, 89099, 89100) c7(eJ, bE, LC, Y, if, qK, cn, r, cy, aj, dG, j, ci, ade, qN, fr, IC, B, J, cp, uX); #c1(ST3GAL4) c2(NP_001241688) c3(CMP-N-acetylneuraminate-beta-galactosamide-alpha-2,3-sialyltransferase 4 isoform 3 precursor) c4(89101) c5(89102, 89103, 89104, 89105, 89106, 89107, 89108, 89109, 89110, 89111, 89112, 89113, 89114, 89115, 89116, 89117) c6(89118, 89119, 89120, 89121) c7(eJ, bE, LC, Y, if, qK, cn, r, cy, aj, dG, j, ci, ade, qN, fr, lC, B, J, cp, uX); #c1(ST3GAL4) c2(NP_006269) c3(CMP-N-acetylneuraminate-beta-galactosamide-alpha-2,3-sialyltransferase 4 isoform 1 precursor) c4(163451, 163452) c5(163453, 163454, 163455, 163456, 163457, 163458, 163459, 163460, 163461, 163462, 163463, 163464, 163465, 163466) c6(163467, 163468) c7(eJ, bE, LC, Y, if, qK, cn, r, cy, aj, dG, j, ci, ade, qN, fr, lC, B, J, cp, uX); #c1(ST3GAL5) c2(NP_001035902) c3(lactosylceramide alpha-2,3-sialyltransferase isoform 2) c4(33346) c5(33347, 33348, 33349, 33350, 33351, 33352, 33353, 33354, 33355, 33356, 33357) c6( ) c7(y, ha, ey, m, t, cJe); #c1(ST3GAL5) c2(NP_003887) c3(lactosylceramide alpha-2,3-sialyltransferase isoform 1) c4(141827, 141828, 141829) c5(141830, 141831, 141832, 141833, 141834, 141835, 141836) c6( ) c7(y, ha, ey, m, t, cJe); #c1(ST3GAL5) c2(XP_005264687) c3 (lactosylceramide alpha-2,3-sialyltransferase isoform X1) c4(279193, 279194) c5(279195, 279196, 279197, 279198, 279199, 279200, 279201, 279202, 279203, 279204, 279205, 279206) c6( ) c7(y, ha, ey, m, t, cJe); #c1(ST3GAL5) c2(XP_011531445) c3(lactosylceramide alpha-2,3-sialyltransferase isoform X2) c4( ) c5(327579, 327580, 327581, 327582, 327583, 327584) c6( ) c7(y, ha, ey, m, t, cJe); #c1(ST3GAL6) c2(NP_001258074) c3(type 2 lactosamine alpha-2,3-sialyltransferase isoform 3) c4(95922) c5(95923) c6( ) c7(T, aA, fd, v, aF, ci, m, t, B); #c1(ST3GAL6) c2(NP_001258075) c3(type 2 lactosamine alpha-2,3-sialyltransferase isoform 1) c4(95924) c5(95925, 95926, 95927, 95928, 95929, 95930, 95931, 95932, 95933, 95934, 95935, 95936, 95937, 95938) c6( ) c7(T, aA, fd, v, aF, ci, m, t, B); #c1(ST3GAL6) c2(NP_001258076) c3(type 2 lactosamine alpha-2,3-sialyltransferase isoform 4) c4(95939) c5(95940, 95941, 95942, 95943, 95944) c6( ) c7(T, aA, fd, v, aF, ci, m, t, B); #c1(ST3GAL6) c2(XP_005247124) c3 (type 2 lactosamine alpha-2,3-sialyltransferase isoform X3) c4(267687) c5(267688, 267689) c6( ) c7(T, aA, fd, v, aF, ci, m, t, B); #c1(ST3GAL6) c2(XP_006713537) c3(type 2 lactosamine alpha-2,3-sialyltransferase isoform X4) c4( ) c5(287222) c6( ) c7(T, aA, fd, v, aF, ci, m, t, B); #c1(ST3GAL6) c2(XP_011510637) c3(type 2 lactosamine alpha-2,3-sialyltransferase isoform X6) c4( ) c5(301085, 301086, 301087, 301088, 301089, 301090, 301091, 301092) c6( ) c7(T, aA, fd, v, aF, ci, m, t, B); #c1(ST5) c2(NP_005409) c3(suppression of tumorigenicity 5 protein isoform 1) c4(155807, 155808) c5(155809, 155810, 155811, 155812, 155813, 155814, 155815, 155816, 155817, 155818, 155819, 155820, 155821, 155822, 155823, 155824, 155825) c6( ) c7(hb); #c1(ST5) c2(NP_631896) c3(suppression of tumorigenicity 5 protein isoform 2) c4(238202, 238203, 238204, 238205, 238206, 238207) c5(238208, 238209, 238210, 238211, 238212, 238213, 238214, 238215, 238216, 238217, 238218, 238219, 238220, 238221, 238222, 238223, 238224, 238225, 238226, 238227, 238228, 238229, 238230, 238231, 238232, 238233, 238234, 238235, 238236, 238237, 238238, 238239, 238240, 238241, 238242, 238243, 238244, 238245, 238246, 238247, 238248, 238249, 238250, 238251, 238252, 238253, 238254, 238255) c6( ) c7(hb); #c1(ST5) c2(XP_005253134) c3(suppression of tumorigenicity 5 protein isoform X5) c4( ) c5(272175, 272176, 272177) c6( ) c7(hb); #c1(ST5) c2(XP_011518611) c3(suppression of tumorigenicity 5 protein isoform X3) c4(312827, 312828, 312829, 312830, 312831) c5(312832, 312833, 312834, 312835, 312836, 312837, 312838, 312839, 312840, 312841, 312842, 312843, 312844, 312845, 312846) c6( ) c7(hb); #c1(ST5) c2(XP_011518612) c3(suppression of tumorigenicity 5 protein isoform X4) c4(312847, 312848, 312849, 312850, 312851, 312852) c5(312853) c6( ) c7(hb); #c1(ST5) c2(XP_011518619) c3(suppression of tumorigenicity 5 protein isoform X6) c4(312854, 312855, 312856) c5( ) c6( ) c7(hb); #c1(ST5) c2(XP_011518624) c3(suppression of tumorigenicity 5 protein isoform X10) c4(312857, 312858) c5(312859, 312860, 312861, 312862, 312863, 312864, 312865, 312866, 312867, 312868, 312869, 312870, 312871, 312872, 312873, 312874, 312875, 312876, 312877, 312878, 312879, 312880, 312881, 312882, 312883, 312884, 312885, 312886, 312887, 312888, 312889, 312890, 312891, 312892, 312893, 312894, 312895, 312896, 312897, 312898, 312899, 312900, 312901, 312902, 312903, 312904, 312905, 312906, 312907, 312908, 312909, 312910, 312911, 312912, 312913, 312914, 312915, 312916, 312917, 312918, 312919, 312920, 312921, 312922, 312923, 312924, 312925, 312926, 312927, 312928, 312929, 312930, 312931, 312932, 312933, 312934, 312935, 312936, 312937, 312938, 312939, 312940, 312941, 312942, 312943) c6( ) c7(hb); #c1(ST5) c2(XP_011518630) c3(suppression of tumorigenicity 5 protein isoform X11) c4(312944, 312945, 312946) c5( ) c6( ) c7(hb); #c1(ST5) c2(XP_011518632) c3(suppression of tumorigenicity 5 protein isoform X12) c4( ) c5(312947, 312948, 312949, 312950, 312951, 312952, 312953, 312954, 312955, 312956) c6( ) c7(hb); #c1(ST6GAL1) c2(NP_775323) c3(beta-galactoside alpha-2,6-sialyltransferase 1 isoform a) c4(249957, 249958, 249959, 249960, 249961, 249962, 249963, 249964, 249965, 249966, 249967) c5(249968, 249969, 249970, 249971, 249972, 249973, 249974, 249975, 249976, 249977, 249978, 249979, 249980, 249981, 249982, 249983, 249984, 249985, 249986, 249987, 249988, 249989, 249990, 249991, 249992, 249993, 249994, 249995, 249996, 249997, 249998, 249999, 250000, 250001, 250002, 250003, 250004, 250005, 250006, 250007, 250008, 250009, 250010, 250011, 250012, 250013, 250014, 250015, 250016, 250017, 250018, 250019, 250020, 250021, 250022, 250023, 250024, 250025, 250026, 250027, 250028, 250029, 250030, 250031, 250032, 250033, 250034, 250035, 250036, 250037, 250038, 250039, 250040, 250041, 250042, 250043, 250044, 250045, 250046, 250047) c6(250048, 250049, 250050, 250051, 250052, 250053, 250054) c7(bL, T, if, al, aA, bB, ci, qN, q, eA, hD, ey, eL, oV, ev, ff, hl, r, aF, en, g, t, pb, ck, gk, bE, m, lC, B, cn); #c1(ST6GAL1) c2(NP_775324) c3(beta-galactoside alpha-2,6-sialyltransferase 1 isoform b) c4( ) c5(250055, 250056, 250057, 250058, 250059, 250060, 250061) c6( ) c7(bL, T, if, al, aA, bB, ci, qN, q, eA, hD, ey, eL, oV, ev, ff, hl, r, aF, en, g, t, pb, ck, gk, bE, m, lC, B, cn); #c1(ST6GAL2) c2(NP_001135824) c3(beta-galactoside alpha-2,6-sialyltransferase 2 isoform b) c4(53356) c5(53357, 53358, 53359, 53360, 53361, 53362) c6( ) c7(aw, j, eA); #c1(ST6GAL2) c2(NP_115917) c3(beta-galactoside alpha-2,6-sialyltransferase 2 isoform a) c4(224675, 224676, 224677) c5(224678, 224679, 224680, 224681, 224682, 224683, 224684, 224685, 224686, 224687, 224688, 224689, 224690, 224691, 224692, 224693) c6( ) c7(aw, j, eA); #c1(ST6GAL2) c2(XP_011510305) c3(beta-galactoside alpha-2,6-sialyltransferase 2 isoform X5) c4( ) c5(300879, 300880, 300881, 300882, 300883, 300884) c6( ) c7(aw, j, eA); #c1(ST6GALNAC1) c2(NP_060884) c3(alpha-N-acetylgalactosaminide alpha-2,6-sialyltransferase 1 isoform 1) c4(198366) c5(198367, 198368, 198369, 198370, 198371, 198372, 198373, 198374, 198375) c6(198376, 198377) c7(T, aT, gl, dL, bK, el); #c1(ST6GALNAC1) c2(XP_011523297) c3(alpha-N-acetylgalactosaminide alpha-2,6-sialyltransferase 1 isoform X1) c4(317812) c5( ) c6( ) c7(T, aT, gl, dL, bK, el); #c1(ST6GALNAC1) c2(XP_011523298) c3(alpha-N-acetylgalactosaminide alpha-2,6-sialyltransferase 1 isoform X2) c4( ) c5(317813, 317814, 317815, 317816, 317817) c6( ) c7(T, aT, gl, dL, bK, el); #c1(ST6GALNAC2) c2(NP_006447) c3(alpha-N-acetylgalactosaminide alpha-2,6-sialyltransferase 2 precursor) c4(165309) c5(165310, 165311, 165312, 165313, 165314, 165315) c6( ) c7(aW, T, ey, aT); #c1(ST6GALNAC4) c2(NP_778204) c3(alpha-N-acetyl-neuraminyl-2,3-beta-galactosyl-1,3-N-acetyl-galactosaminide alpha-2,6-sialyltransferase isoform a) c4(251382, 251383) c5(251384, 251385, 251386, 251387, 251388, 251389, 251390) c6( ) c7(DY); #c1(ST6GALNAC4) c2(NP_778205) c3(alpha-N-acetyl-neuraminyl-2,3-beta-galactosyl-1,3-N-acetyl-galactosaminide alpha-2,6-sialyltransferase isoform b) c4(251391, 251392) c5(251393, 251394, 251395, 251396, 251397, 251398, 251399, 251400, 251401, 251402, 251403, 251404, 251405) c6( ) c7(DY); #c1(ST6GALNAC4) c2(XP_011516829) c3(alpha-N-acetyl-neuraminyl-2,3-beta-galactosyl-1,3-N-acetyl-galactosaminide alpha-2,6-sialyltransferase isoform X1) c4( ) c5(310265, 310266, 310267) c6( ) c7(DY); #c1(ST6GALNAC6) c2(NP_001273928) c3(alpha-N-acetylgalactosaminide alpha-2,6-sialyltransferase 6 isoform b) c4(105903, 105904) c5(105905, 105906, 105907, 105908, 105909, 105910) c6( ) c7(es, T, iA); #c1(ST6GALNAC6) c2(NP_001273930) c3(alpha-N-acetylgalactosaminide alpha-2,6-sialyltransferase 6 isoform c) c4(105911, 105912) c5(105913, 105914, 105915, 105916, 105917, 105918, 105919, 105920, 105921, 105922, 105923, 105924, 105925) c6( ) c7(es, T, iA); #c1(ST6GALNAC6) c2(NP_038471) c3(alpha-N-acetylgalactosaminide alpha-2,6-sialyltransferase 6 isoform a) c4(176585) c5(176586, 176587, 176588, 176589, 176590, 176591, 176592, 176593, 176594, 176595, 176596, 176597, 176598) c6( ) c7(es, T, iA); #c1(ST6GALNAC6) c2(XP_011516916) c3(alpha-N-acetylgalactosaminide alpha-2,6-sialyltransferase 6 isoform X3) c4( ) c5(310428, 310429, 310430, 310431, 310432, 310433) c6( ) c7(es, T, iA); #c1(ST7) c2(NP_060882) c3(suppressor of tumorigenicity 7 protein isoform a) c4(198320, 198321, 198322) c5(198323, 198324, 198325, 198326, 198327, 198328, 198329, 198330, 198331, 198332, 198333, 198334, 198335, 198336, 198337, 198338, 198339, 198340, 198341, 198342, 198343, 198344, 198345, 198346, 198347, 198348, 198349, 198350, 198351, 198352, 198353, 198354, 198355, 198356, 198357, 198358) c6( ) c7(T, B, bE, aW, r, aT, bB, ci, hu, q); #c1(ST7) c2(NP_068708) c3(suppressor of tumorigenicity 7 protein isoform b) c4(210084, 210085, 210086, 210087) c5(210088, 210089, 210090, 210091, 210092, 210093) c6( ) c7(T, B, bE, aW, r, aT, bB, ci, hu, q); #c1(ST7L) c2(XP_011539931) c3(suppressor of tumorigenicity 7 protein-like isoform X3) c4(338276, 338277) c5(338278, 338279, 338280, 338281, 338282, 338283, 338284, 338285, 338286, 338287, 338288, 338289) c6( ) c7(T, aF, eA, aA); #c1(ST8SIA1) c2(NP_001291379) c3(alpha-N-acetylneuraminide alpha-2,8-sialyltransferase isoform 2) c4(115809) c5(115810, 115811, 115812, 115813, 115814) c6( ) c7(ck, av, dD, iU, qt, aW, Y, oV, hl, al, eq, V, iq); #c1(ST8SIA1) c2(NP_003025) c3(alpha-N-acetylneuraminide alpha-2,8-sialyltransferase isoform 1) c4(134530) c5(134531, 134532, 134533, 134534, 134535, 134536, 134537) c6( ) c7(ck, av, dD, il, qt, aW, Y, oV, hl, aT, eq, V, iq); #c1(ST8SIA2) c2(NP_006002) c3(alpha-2,8-sialyltransferase 88 precursor) c4(161102, 161103) c5(161104, 161105, 161106, 161107, 161108, 161109, 161110, 161111, 161112, 161113, 161114, 161115) c6( ) c7(T, cA, eM, ql, cm, BF, bK, he, cd, bN); #c1(ST8SIA3) c2(NP_056963) c3(sia-alpha-2,3-Gal-beta-1,4-GlcNAc-R:alpha 2,8-sialyltransferase) c4(188220, 188221) c5(188222, 188223, 188224, 188225, 188226, 188227, 188228, 188229, 188230, 188231, 188232, 188233) c6( ) c7(j); #c1(ST8SIA4) c2(NP_005659) c3(CMP-N-acetylneuraminate-poly-alpha-2, 8-sialyltransferase isoform a precursor) c4(158333, 158334) c5(158335, 158336, 158337, 158338, 158339, 158340) c6( ) c7(bL, T, jv, eR, QO, oe, cmu, cz, vr, j, ci, q, ad, kL, afR, l, aT, io, h, aaW, hB, ae, dF, Yi, sj, Fm, ff, r, fe, uY, aq, Ku, en, cm, uC, bz, fq, qa, rR, VJ, ac, mg, uz, Gp, B); #c1(ST8SIA4) c2(NP_778222) c3(CMP-N-acetylneuraminate-poly-alpha-2,8-sialyltransferase isoform b precursor) c4( ) c5(251413, 251414, 251415, 251416, 251417, 251418) c6( ) c7(bL, T, jv, eR, QO, oe, cmu, cz, vr, j, ci, q, ad, kL, afR, l, aT, io, h, aaW, hB, ae, dF, Yi, sj, Fm, ff, r, fe, uY, aq, Ku, en, cm, uC, bz, fq, qa, rR, VJ, ac, mg, uz, Gp, B); #c1(ST8SIA6) c2(NP_001004470) c3(alpha-2,8-sialyltransferase 8F precursor) c4(20766, 20767, 20768) c5(20769, 20770, 20771, 20772, 20773, 20774, 20775) c6( ) c7(k); #c1(ST8SIA6) c2(XP_011517754) c3(alpha-2,8-sialyltransferase 8F isoform X1) c4( ) c5(311587, 311588, 311589, 311590, 311591, 311592, 311593, 311594, 311595, 311596, 311597) c6( ) c7(k); #c1(STAB1) c2(NP_055951) c3(stabilin-1 precursor) c4(184638) c5(184639, 184640, 184641, 184642, 184643, 184644) c6( ) c7(dy, B); #c1(STAB1) c2(XP_005265032) c3(stabilin-1 isoform X4) c4( ) c5(279398, 279399, 279400, 279401, 279402) c6( ) c7(dy, B); #c1(STAB2) c2(NP_060034) c3(stabilin-2 precursor) c4(193797) c5(193798, 193799, 193800, 193801, 193802, 193803, 193804, 193805) c6( ) c7(ac, fc, GR, j, cP, B); #c1(STAB2) c2(XP_011536843) c3(stabilin-2 isoform X5) c4(334534) c5( ) c6( ) c7(ac, fc, GR, j, cP, B); #c1(STAB2) c2(XP_011536844) c3(stabilin-2 isoform X6) c4( ) c5(334535, 334536, 334537, 334538, 334539, 334540) c6( ) c7(ac, fc, GR, j, cP, B); #c1(STAC) c2(NP_003140) c3(SH3 and cysteine-rich domain-containing protein isoform 1) c4(135633, 135634) c5(135635, 135636, 135637, 135638, 135639, 135640, 135641) c6( ) c7(oe, fJ); #c1(STAC) c2(XP_011532340) c3(SH3 and cysteine-rich domain-containing protein isoform X3) c4( ) c5(328489, 328490, 328491, 328492, 328493) c6( ) c7(oe, fJ); #c1(STAC3) c2(NP_001273185) c3(SH3 and cysteine-rich domain-containing protein 3 isoform b) c4( ) c5(104514, 104515, 104516, 104517, 104518) c6( ) c7(cJf); #c1(STAC3) c2(NP_659501) c3(SH3 and cysteine-rich domain-containing protein 3 isoform a) c4(240254, 240255, 240256) c5(240257, 240258, 240259, 240260, 240261, 240262, 240263, 240264, 240265, 240266, 240267, 240268, 240269) c6( ) c7(cJf); #c1(STAG1) c2(NP_005853) c3(cohesin subunit SA-1) c4(159890, 159891) c5(159892, 159893, 159894, 159895, 159896, 159897, 159898, 159899, 159900, 159901, 159902, 159903, 159904, 159905, 159906, 159907, 159908, 159909, 159910, 159911, 159912, 159913, 159914, 159915) c6( ) c7(fr, K, m, t, ae, ad); #c1(STAG1) c2(XP_011510634) c3(cohesin subunit SA-1 isoform X2) c4(301083) c5( ) c6( ) c7(fr, K, m, t, ae, ad); #c1(STAG1) c2(XP_011510635) c3(cohesin subunit SA-1 isoform X1) c4(301084) c5( ) c6( ) c7(fr, K, m, t, ae, ad); #c1(STAG2) c2(NP_001036215) c3(cohesin subunit SA-2 isoform a) c4(34063, 34064, 34065, 34066, 34067, 34068, 34069, 34070) c5(34071, 34072, 34073, 34074, 34075, 34076, 34077, 34078, 34079, 34080, 34081, 34082, 34083, 34084, 34085, 34086, 34087, 34088, 34089, 34090, 34091) c6( ) c7(T, d, id, c, iT, eL, SV, dL, ci, g, aYh); #c1(STAG2) c2(NP_001269347) c3(cohesin subunit SA-2 isoform b) c4(101183, 101184) c5(101185, 101186, 101187, 101188, 101189, 101190, 101191, 101192, 101193, 101194, 101195, 101196, 101197, 101198, 101199, 101200, 101201, 101202, 101203, 101204, 101205, 101206, 101207, 101208, 101209, 101210, 101211, 101212, 101213, 101214, 101215, 101216, 101217, 101218, 101219, 101220, 101221, 101222, 101223, 101224, 101225, 101226, 101227, 101228, 101229, 101230, 101231, 101232, 101233, 101234, 101235) c6( ) c7(T, d, id, c, iT, eL, SV, dL, ci, g, aYh); #c1(STAG3) c2(NP_001269646) c3(cohesin subunit SA-3 isoform 2) c4( ) c5(102383, 102384, 102385) c6( ) c7(t, gU, m, cJg); #c1(STAG3) c2(NP_001269647) c3(cohesin subunit SA-3 isoform 3) c4(102386, 102387, 102388, 102389, 102390, 102391) c5(102392, 102393, 102394, 102395, 102396, 102397, 102398, 102399, 102400, 102401, 102402, 102403, 102404, 102405) c6( ) c7(t, gU, m, cJg); #c1(STAG3) c2(NP_036579) c3(cohesin subunit SA-3 isoform 1) c4(174492, 174493, 174494, 174495, 174496, 174497) c5(174498, 174499, 174500, 174501, 174502, 174503, 174504, 174505, 174506, 174507, 174508, 174509, 174510, 174511, 174512, 174513) c6( ) c7(t, gU, m, cJg); #c1(STAG3) c2(XP_005250173) c3(cohesin subunit SA-3 isoform X2) c4(270160) c5(270161, 270162, 270163, 270164, 270165) c6( ) c7(t, gU, m, cJg); #c1(STAG3) c2(XP_011514046) c3(cohesin subunit SA-3 isoform X3) c4(306497, 306498, 306499, 306500) c5(306501, 306502, 306503, 306504, 306505, 306506, 306507, 306508, 306509, 306510, 306511, 306512, 306513, 306514, 306515, 306516, 306517, 306518, 306519, 306520, 306521, 306522, 306523, 306524, 306525, 306526, 306527, 306528, 306529, 306530, 306531, 306532, 306533, 306534, 306535, 306536, 306537, 306538, 306539, 306540, 306541, 306542, 306543, 306544, 306545, 306546, 306547, 306548, 306549, 306550, 306551, 306552, 306553, 306554, 306555) c6( ) c7(t, gU, m, cJg); #c1(STAG3) c2(XP_011514047) c3(cohesin subunit SA-3 isoform X4) c4( ) c5(306556, 306557, 306558, 306559, 306560, 306561, 306562, 306563, 306564, 306565, 306566, 306567, 306568, 306569, 306570, 306571, 306572, 306573, 306574, 306575, 306576, 306577, 306578, 306579, 306580, 306581, 306582, 306583, 306584) c6( ) c7(t, gU, m, cJg); #c1(STAM) c2(NP_003464) c3(signal transducing adapter molecule 1) c4(138547) c5(138548, 138549, 138550, 138551, 138552, 138553, 138554) c6( ) c7(B, al, fc); #c1(STAM) c2(XP_005252660) c3(signal transducing adapter molecule 1 isoform X2) c4(271956, 271957, 271958) c5(271959, 271960, 271961, 271962, 271963, 271964, 271965, 271966, 271967, 271968, 271969, 271970, 271971, 271972, 271973, 271974, 271975) c6( ) c7(B, al, fc); #c1(STAM2) c2(NP_005834) c3(signal transducing adapter molecule 2) c4(159771) c5(159772, 159773, 159774, 159775, 159776, 159777) c6( ) c7(dc, T, ad); #c1(STAMBP) c2(NP_998787) c3(STAM-binding protein) c4(265974, 265975, 265976) c5(265977, 265978, 265979, 265980, 265981, 265982, 265983, 265984, 265985, 265986, 265987, 265988, 265989, 265990, 265991, 265992, 265993, 265994) c6( ) c7(cyw, hb, ha, aFy, cJh, QK); #c1(STAMBP) c2(XP_011530787) c3(STAM-binding protein isoform X3) c4(326813, 326814) c5(326815, 326816, 326817, 326818, 326819, 326820, 326821, 326822, 326823, 326824, 326825, 326826, 326827, 326828, 326829, 326830, 326831, 326832, 326833, 326834, 326835, 326836, 326837, 326838) c6( ) c7(cyw, hb, ha, aFy, cJh, QK); #c1(STAMBPL1) c2(NP_065850) c3(AMSH-like protease) c4(205850, 205851, 205852) c5(205853, 205854, 205855, 205856, 205857, 205858, 205859, 205860, 205861, 205862, 205863, 205864, 205865, 205866, 205867, 205868, 205869, 205870, 205871, 205872, 205873, 205874) c6( ) c7(iM, je, cd); #c1(STAP1) c2(NP_036240) c3(signal-transducing adaptor protein 1) c4(172584) c5(172585, 172586, 172587, 172588, 172589, 172590, 172591, 172592, 172593, 172594) c6( ) c7(c1, an); #c1(STAP2) c2(NP_001013863) c3(signal-transducing adaptor protein 2 isoform 2) c4(25789) c5(25790, 25791, 25792, 25793, 25794, 25795) c6( ) c7(aW, cF, iU, aT); #c1(STAP2) c2(NP_060190) c3(signal-transducing adaptor protein 2 isoform 1) c4(194668, 194669, 194670, 194671) c5(194672, 194673, 194674, 194675, 194676, 194677, 194678, 194679, 194680, 194681, 194682, 194683, 194684, 194685, 194686, 194687, 194688, 194689, 194690, 194691, 194692, 194693, 194694, 194695, 194696, 194697) c6( ) c7(aW, cF, il, aT); #c1(STAP2) c2(XP_011526425) c3(signal-transducing adaptor protein 2 isoform X1) c4( ) c5(320727, 320728, 320729, 320730, 320731, 320732, 320733, 320734, 320735, 320736) c6( ) c7(aW, cF, iU, aT); #c1(STAR) c2(NP_000340) c3(steroidogenic acute regulatory protein, mitochondrial) c4(7410, 7411) c5(7412, 7413, 7414, 7415, 7416, 7417, 7418, 7419, 7420, 7421, 7422, 7423) c6( ) c7(bL, gh, T, aZ, iW, baV, jN, bO, cz, n, bS, ct, Fc, eA, ad, UE, qC, dP, ax, BJ, iv, h, bK, bq, hK, ae, af, ak, kl, avH, gP, aW, Hu, cl, Z, t, baW, aqp, hl, Rm, va, m, UF, gT); #c1(STARD10) c2(NP_006636) c3(PCTP-like protein) c4(166776, 166777, 166778, 166779, (66780) c5(166781, 166782, 166783, 166784, 166785, 166786, 166787, 166788, 166789, 166790, 166791, 166792, 166793, 166794, 166795, 166796, 166797, 166798, 166799, 166800, 166801, 166802, 166803, 166804, 166805, 166806, 166807, 166808, 166809, 166810, 166811, 166812, 166813, 166814, 166815, 166816, 166817, 166818) c6( ) c7(aW, ad, aT); #c1(STARD13) c2(NP_001230395) c3(stAR-related lipid transfer protein 13 isoform 5) c4( ) c5(86268, 86269, 86270, 86271, 86272, 86273) c6( ) c7(T, d, pN, iR, aW, Y, hL, aT, aq, ci, K, nA, as, B, us, cl); #c1(STARD13) c2(NP_821074) c3(stAR-related lipid transfer protein 13 isoform 1) c4(253177) c5(253178, 253179, 253180, 253181, 253182, 253183, 253184) c6( ) c7(T, d, pN, iR, aW, Y, hl, aT, aq, ci, K, nA, as, B, us, cl); #c1(STARD13) c2(NP_821075) c3(stAR-related lipid transfer protein 13 isoform 2) c4(253185, 253186) c5(253187, 253188, 253189, 253190, 253191, 253192, 253193, 253194, 253195, 253196, 253197) c6( ) c7(T, d, pN, iR, aW, Y, hL, aT, aq, ci, K, nA, as, B, us, cl); #c1(STARD13) c2(XP_011533600) c3(stAR-related lipid transfer protein 13 isoform X2) c4(329711) c5( ) c6( ) c7(T, d, pN, iR, aW, Y, hL, aT, aq, ci, K, nA, as, B, us, cl); #c1(STARD3) c2(NP_001159409) c3(stAR-related lipid transfer protein 3 isoform 2) c4(63970) c5(63971, 63972, 63973, 63974, 63975, 63976) c6( ) c7(T, aW, aA, aT, aF, ae, ad); #c1(STARD3) c2(NP_001159410) c3(stAR-related lipid transfer protein 3 isoform 3) c4(63977) c5(63978, 63979, 63980, 63981, 63982, 63983) c6( ) c7(T, aW, aA, aT, aF, ae, ad); #c1(STARD3) c2(NP_006795) c3(stAR-related lipid transfer protein 3 isoform 1) c4(168140, 168141) c5(168142, 168143, 168144, 168145, 168146, 168147, 168148, 168149, 168150, 168151, 168152, 168153) c6( ) c7(T, aW, aA, aT, aF, ae, ad); #c1(STARD3NL) c2(NP_114405) c3(MLN64 N-terminal domain homolog) c4(222552, 222553, 222554, 222555, 222556) c5(222557, 222558, 222559, 222560, 222561, 222562, 222563, 222564, 222565, 222566, 222567, 222568, 222569, 222570, 222571, 222572, 222573, 222574, 222575, 222576, 222577, 222578, 222579, 222580, 222581, 222582, 222583, 222584, 222585, 222586, 222587, 222588, 222589, 222590, 222591, 222592) c6( ) c7(iW, C); #c1(STARD5) c2(NP_871629) c3(stAR-related lipid transfer protein 5) c4(255947) c5(255948, 255949, 255950, 255951, 255952, 255953) c6( ) c7(K); #c1(STARD7) c2(NP_064536) c3(stAR-related lipid transfer protein 7, mitochondrial precursor) c4(202885) c5(202886, 202887, 202888, 202889, 202890, 202891) c6( ) c7(nJ, B); #c1(STARD8) c2(NP_001135975) c3(stAR-related lipid transfer protein 8 isoform a) c4(53898) c5(53899, 53900, 53901, 53902, 53903, 53904, 53905, 53906, 53907, 53908, 53909, 53910, 53911, 53912, 53913, 53914, 53915) c6( ) c7(T, aW, aT, ci, ae, ad); #c1(STARD8) c2(NP_055540) c3(stAR-related lipid transfer protein 8 isoform b) c4(181832) c5( ) c6( ) c7(T, aW, aT, ci, ae, ad); #c1(STARD8) c2(XP_005262371) c3(stAR-related lipid transfer protein 8 isoform X2) c4(277861) c5(277862, 277863, 277864, 277865, 277866) c6( ) c7(T, aW, aT, ci, ae, ad); #c1(STARD9) c2(NP_065810) c3(stAR-related lipid transfer protein 9) c4(205642) c5(205643, 205644, 205645, 205646, 205647, 205648) c6( ) c7(ac); #c1(STARD9) c2(XP_011520141) c3(stAR-related lipid transfer protein 9 isoform X9) c4( ) c5(315021, 315022) c6( ) c7(ac); #c1(STAT1) c2(NP_009330) c3(signal transducer and activator of transcription 1-alpha/beta isoform alpha) c4(171827, 171828, 171829, 171830, 171831, 171832, 171833, 171834) c5(171835, 171836, 171837, 171838, 171839, 171840, 171841, 171842, 171843, 171844, 171845, 171846) c6( ) c7(oO, bM, T, os, fR, ea, aAx, al, fd, bB, ci, x, bOl, aE, Bc, ja, Ds, azm, K, eL, cp, eG, cJj, aW, apn, g, cm, bw, ba, eM, nB, iT, aiM, cj, k, Hb, m, dD, akc, fN, n, Mi, fr, MT, qW, pe, kH, cU, lG, aDa, dL, bOp, DU, bQd, jy, ey, h, pi, afu, A, aq, CQ, beP, pm, jT, RB, Pa, dB, Qu, ln, bm, at, fL, it, B, aMk, Dw, bli, lk, fG, jx, aA, cR, C, V, pd, q, er, eA, xx, mj, bNv, aT, aAn, cy, jj, bq, Cy, ae, bA, bf, il, nO, Y, fO, bg, r, lo, en, t, f, ik, ck, cT, vK, iM, kP, ac, hc, wR, CZ, gT, bt, zA, zu, ao, vi, gV, bxK, jh, oZ, eq, jE, EA, hF, mC, ad, cJi, hX, qC, F, ib, afm, ds, oW, nJ, atX, d, je, ff, eZ, aF, hp, Fq, anW, iV, bz, wW, gk, Bv, zr, bE, aS, lf, KS, X, y); #c1(STAT1) c2(NP_644671) c3(signal transducer and activator of transcription 1-alpha/beta isoform beta) c4( ) c5(238490, 238491, 238492, 238493, 238494, 238495, 238496, 238497, 238498, 238499, 238500, 238501, 238502, 238503, 238504, 238505, 238506, 238507, 238508, 238509, 238510, 238511, 238512, 238513, 238514, 238515, 238516, 238517, 238518, 238519, 238520, 238521, 238522, 238523, 238524, 238525, 238526, 238527, 238528, 238529, 238530, 238531, 238532, 238533, 238534) c6( ) c7(oO, bM, T, os, fR, ea, aAx, al, fd, bB, ci, x, bOl, aE, Bc, ja, Ds, azm, K, eL, cp, eG, cJj, aW, apn, g, cm, bw, ba, eM, nB, iT, aiM, cj, k, Hb, m, dD, akc, fN, n, Mi, fr, MT, qW, pe, kH, cU, lG, aDa, dL, bOp, DU, bQd, jy, ey, h, pi, afu, A, aq, CQ, beP, pm, jT, RB, Pa, dB, Qu, ln, bm, at, fL, it, B, aMk, Dw, bli, lk, fG, jx, aA, cR, C, V, pd, q, er, eA, xx, mj, bNv, aT, aAn, cy, jj, bq, Cy, ae, bA, bf, il, nO, Y, fO, bg, r, lo, en, t, f, ik, ck, cT, vK, iM, kP, ac, hc, wR, CZ, gT, bt, zA, zu, ao, vi, gV, bxK, jh, oZ, eq, jE, EA, hF, mC, ad, cJi, hX, qC, F, ib, afm, ds, oW, nJ, atX, d, je, ff, eZ, aF, hp, Fq, anW, iV, bz, wW, gk, Bv, zr, bE, aS, lf, KS, X, y); #c1(STAT2) c2(NP_005410) c3(signal transducer and activator of transcription 2 isoform 1) c4(155826) c5(155827, 155828, 155829, 155830, 155831, 155832, 155833) c6( ) c7(gV, T, bM, al, jx, bB, EA, V, F, ja, aT, cU, lG, h, DU, jj, hG, aW, Y, cr, eZ, aq, lo, CQ, W, iT, bE, aiM, B); #c1(STAT2) c2(NP_938146) c3(signal transducer and activator of transcription 2 isoform 2) c4(259617) c5(259618, 259619, 259620, 259621, 259622, 259623) c6( ) c7(gV, T, bM, al, jx, bB, EA, V, F, ja, aT, cU, lG, h, DU, jj, hG, aW, Y, cr, eZ, aq, lo, CQ, W, iT, bE, aiM, B); #c1(STAT2) c2(XP_005269168) c3(signal transducer and activator of transcription 2 isoform X7) c4(282382) c5(282383, 282384, 282385, 282386, 282387, 282388) c6( ) c7(gV, T, bM, al, jx, bB, EA, V, F, ja, aT, cU, lG, h, DU, jj, hG, aW, Y, cr, eZ, aq, lo, CQ, W, iT, bE, aiM, B); #c1(STAT3) c2(NP_003141) c3(signal transducer and activator of transcription 3 isoform 2) c4(135642) c5(135643, 135644, 135645, 135646, 135647, 135648, 135649) c6( ) c7(oO, bM, T, lD, aC, fR, tQ, hV, ea, if, e, al, Xi, yS, pf, bB, fd, ci, aY, amW, ip, x, xy, xE, aE, Xc, Bc, ja, DO, Ds, K, qM, hu, hH, dF, bc, eL, cp, aa, ly, zQ, ZH, Jl, aW, Ls, az, HA, fc, CT, vs, g, as, eE, eF, cm, baR, Qt, bw, ou, eM, nB, xD, lq, iA, iT, mx, U, cj, aep, wB, uJ, dW, k, Hb, m, hh, aDM, qm, ik, bhp, iu, xN, aiu, dD, sw, aw, ht, NT, fN, qy, Bm, n, aj, fr, qN, aOJ, zP, qW, cd, br, asS, ai, md, eW, ft, jz, cc, aoc, aeX, gd, kH, h, crs, jy, dL, bl, eV, asT, dx, cY, A, mR, CN, Cw, CM, blP, fe, aq, kp, yq, pm, fs, lx, ar, W, dB, hk, hL, at, pg, oh, it, B, bL, Dw, fM, dA, eR, wL, yk, lH, jx, eX, aA, fF, hz, hy, v, j, btX, AO, V, q, J, er, eA, lg, hW, nv, Qg, aoA, qp, afQ, aT, qU, aZd, cy, Fx, jj, bq, ae, af, eu, jf, kl, bf, iU, nO, xR, oV, Y, O, r, afq, lo, en, hf, zM, LV, t, f, cl, ik, bl, ck, Ta, sS, nM, kP, Dy, aQd, iM, Nk, hc, QG, gX, wR, mf, np, yH, dy, fD, CZ, Da, bt, ZA, rA, LS, ao, hS, im, gV, Gl, Ve, Zl, aD, iW, jh, KO, wV, buc, eq, pz, hF, atK, ad, KL, hX, bhq, av, afb, hd, F, ot, ib, Db, py, arV, lC, hD, ji, lz, fb, nJ, pq, yF, aXz, Ol, d, je, hG, qt, cJk, eH, ff, cr, eZ, dq, Ej, aF, iV, Z, iR, KM, bz, qa, bF, es, gk, eU, pc, zr, bE, z, gZ, bC, ei, adg, hE, X, y, Tc); #c1(STAT3) c2(NP_644805) c3(signal transducer and activator of transcription 3 isoform 1) c4(238549, 238550) c5(238551, 238552, 238553, 238554, 238555, 238556, 238557, 238558, 238559, 238560, 238561, 238562, 238563, 238564) c6( ) c7(oO, bM, T, lD, aC, fR, tQ, hV, ea, if, e, al, Xi, yS, pf, bB, fd, ci, aY, amW, ip, x, xy, xE, aE, Xc, Bc, ja, DO, Ds, K, qM, hu, hH, dF, bc, eL, cp, aa, ly, zQ, ZH, Jl, aW, Ls, az, HA, fc, CT, vs, g, as, eE, eF, cm, baR, Qt, bw, ou, eM, nB, xD, lq, iA, iT, mx, U, cj, aep, wB, uJ, dW, k, Hb, m, hh, aDM, qm, ik, bhp, iu, xN, aiu, dD, sw, aw, ht, NT, fN, qy, Bm, n, aj, fr, qN, aOJ, zP, qW, cd, br, asS, ai, md, eW, ft, jz, cc, aoc, aeX, gd, kH, h, crs, jy, dL, bl, eV, asT, dx, cY, A, mR, CN, Cw, CM, blP, fe, aq, kp, yq, pm, fs, lx, ar, W, dB, hk, hl, at, pg, oh, it, B, bL, Dw, fM, dA, eR, wL, yk, lH, jx, eX, aA, fF, hz, hy, v, j, btX, AO, V, q, J, er, eA, lg, hW, nv, Qg, aoA, qp, afQ, aT, qU, aZd, cy, Fx, jj, bq, ae, af, eu, jf, kl, bf, iU, nO, xR, oV, Y, fO, r, afq, lo, en, hf, zM, LV, t, f, cl, ik, bQ, ck, Ta, sS, nM, kP, Dy, and, iM, Nk, hc, QG, gX, wR, mf, np, yH, dy, fD, CZ, Da, bt, zA, rA, LS, ao, hS, im, gV, Gl, Ve, Zl, aD, iW, jh, KO, wV, buc, eq, pz, hF, atK, ad, kL, hX, bhq, av, afb, hd, F, ot, ib, Db, py, arV, lC, HD, ji, lz, fb, nJ, pq, yF, aXz, Ol, d, je, hG, qt, cJk, eH, ff, cr, eZ, dq, Ej, aF, iV, Z, iR, KM, bz, qa, bF, es, gk, eU, pc, zr, bE, z, gZ, bC, ei, adg, hE, X, y, Tc); #c1(STAT3) c2(NP_998827) c3(signal transducer and activator of transcription 3 isoform 3) c4(266057) c5(266058, 266059, 266060, 266061, 266062, 266063) c6( ) c7(oO, bM, T, lD, aC, fR, tQ, hV, ea, if, e, al, Xi, yS, pf, bB, fd, ci, aY, amW, ip, x, xy, xE, aE, Xc, Bc, ja, DO, Ds, K, qM, hu, hH, dF, bc, eL, cp, aa, ly, zQ, ZH, Jl, aW, Ls, az, HA, fc, CT, vs, g, as, eE, eF, cm, baR, Qt, bw, ou, eM, nB, xD, lq, iA, iT, mx, U, cj, aep, wB, uJ, dW, k, Hb, m, hh, aDM, qm, ik, bhp, iu, xN, aiu, dD, sw, aw, ht, NT, fN, qy, Bm, n, aj, fr, qN, aOJ, zP, qW, cd, br, asS, ai, md, eW, ft, jz, cc, aoc, aex, gd, kH, h, crs, jy, dL, bl, eV, asT, dx, cY, A, mR, CN, Cw, CM, blP, fe, aq, kp, yq, pm, fs, lx, ar, W, dB, hk, hl, at, pg, oh, it, B, bL, Dw, fM, dA, eR, wL, yk, lH, jx, eX, aA, fF, hz, hy, v, j, btX, AO, V, q, J, er, eA, lg, hW, nv, Qg, aoA, qp, afQ, aT, qU, aZd, cy, Fx, jj, bq, ae, af, eu, jf, kl, bf, iU, nO, xR, oV, Y, fO, r, afq, lo, en, hf, zM, LV, t, f, cl, ik, bl, ck, Ta, SS, nM, kP, Dy, and, iM, Nk, hc, QG, gX, wR, mf, np, yH, dy, fD, CZ, Da, bt, ZA, rA, LS, ao, hS, im, gV, Gl, Ve, Zl, aD, iW, jh, KO, wV, buc, eq, pz, hF, atK, ad, kL, hX, bhq, av, afb, hd, F, ot, ib, Db, py, arV, IC, hD, ji, lz, fb, nJ, pq, yF, aXz, Ol, d, je, hG, qt, cJk, eH, ff, cr, eZ, dq, Ej, aF, iV, Z, iR, KM, bz, qa, bF, es, gk, eU, pc, zr, bE, z, gZ, bC, ei, adg, hE, X, y, Tc); #c1(STAT4) c2(NP_003142) c3(signal transducer and activator of transcription 4) c4(135650, 135651, 135652, 135653) c5(135654, 135655, 135656, 135657, 135658, 135659, 135660, 135661, 135662, 135663) c6( ) c7(bL, bM, T, dD, Dw, eR, xM, ra, ea, dk, e, yG, fG, fN, aA, cR, oY, cl, v, qj, jE, eq, bh, ez, aY, V, lR, mC, o, cd, ai, dP, hX, eW, wD, bW, aT, Ds, bOp, ey, bay, hH, lM, bc, yF, aEV, OV, A, aW, HA, hl, cr, ge, Pr, ax, eZ, aF, fc, fe, jh, iV, pb, yd, Bv, eU, jx, zr, ig, aCT, cJl, hc, z, aDu, ajd, fP, uo, hr, oh, at, k, Hb, FL, y, Mj, B); #c1(STAT4) c2(XP_011510006) c3(signal transducer and activator of transcription 4 isoform X2) c4( ) c5(300619, 300620, 300621, 300622, 300623, 300624) c6( ) c7(bL, bM, T, dD, Dw, eR, xM, ra, ea, d, e, yG, fG, fN, aA, cR, oY, cl, v, qj, jE, eq, bh, ez, aY, V, lR, mC, o, cd, ai, dP, hX, eW, wD, bW, aT, Ds, bOp, ey, bay, hH, lM, bc, yF, aEV, OV, A, aW, HA, hl, cr, ge, Pr, ax, eZ, aF, fc, fe, jh, iV, pb, yd, Bv, eU, jx, zr, ig, aCT, cJl, hc, z, aDu, ajd, fP, uo, hr, oh, at, k, Hb, fl, y, Mj, B); #c1(STAT4) c2(XP_011510008) c3(signal transducer and activator of transcription 4 isoform X3) c4( ) c5(300625, 300626, 300627, 300628, 300629, 300630, 300631, 300632, 300633, 300634, 300635, 300636, 300637) c6( ) c7(bL, bM, T, dD, Dw, eR, xM, ra, ea, dK, e, yG, fG, fN, aA, cR, oY, cL, v, qj, jE, eq, bh, ez, aY, V, lR, mC, o, cd, ai, dP, hX, eW, wD, bW, aT, Ds, bOp, ey, bay, hH, lM, bc, yF, aEV, OV, A, aW, HA, hl, cr, ge, Pr, ax, eZ, aF, fc, fe, jh, iV, pb, yd, Bv, eU, jx, zr, ig, aCT, cJl, hc, z, aDu, ajd, fP, uo, hr, oh, at, k, Hb, fl, y, Mj, B); #c1(STAT5A) c2(NP_001275648) c3(signal transducer and activator of transcription 5A isoform 2) c4(106888) c5(106889, 106890, 106891, 106892, 106893, 106894, 106895, 106896, 106897) c6( ) c7(bM, T, if, fd, ci, amW, x, Ds, K, cp, zQ, aW, Ls, aGQ, g, eE, cm, bw, eM, iT, cj, k, m, in, Ro, lT, n, aj, qN, zP, bkU, kH, h, lG, eV, bl, ox, A, aq, Og, pm, yR, ok, dB, ln, pg, B, bL, Dw, fM, dA, Dh, jx, eX, afK, hy, j, v, asA, AO, q, J, mj, aT, kf, dz, jj, aPF, ae, iU, oV, r, en, hf, t, ik, ck, Ta, iM, c, bt, aJc, iW, DA, jh, hF, ad, av, btw, bj, d, je, et, ff, xs, Of, eZ, dN, iV, eU, pc, aS, y, iu, Tc); #c1(STAT5A) c2(NP_001275649) c3(signal transducer and activator of transcription 5A isoform 3) c4(106898, 106899) c5(106900, 106901, 106902, 106903, 106904, 106905) c6( ) c7(bM, T, if, fd, ci, amW, x, Ds, K, cp, zQ, aW, Ls, aGQ, g, eE, cm, bw, eM, iT, cj, k, m, in, Ro, lT, n, aj, qN, zP, bkU, kH, h, lG, eV, bl, ox, A, aq, Og, pm, yR, ok, dB, ln, pg, B, bL, Dw, fM, dA, Dh, jx, eX, afK, hy, j, v, asA, AO, q, J, mj, aT, kf, dz, jj, aPF, ae, iU, oV, r, en, hf, t, ik, ck, Ta, iM, c, bt, aJc, iW, oA, jh, hF, ad, av, btw, bj, d, je, et, ff, xs, Of, eZ, dN, iV, eU, pc, aS, y, iu, Tc); #c1(STAT5A) c2(NP_003143) c3(signal transducer and activator of transcription 5A isoform 1) c4(135664, 135665) c5(135666, 135667, 135668, 135669, 135670, 135671, 135672, 135673, 135674, 135675, 135676, 135677, 135678) c6( ) c7(bM, T, if, fd, ci, amW, x, Ds, K, cp, zQ, aW, Ls, aGQ, g, eE, cm, bw, eM, iT, cj, k, m, in, Ro, lT, n, aj, qN, zP, bkU, KH, h, lG, eV, bl, ox, A, aq, Og, pm, yR, ok, dB, ln, pg, B, bL, Dw, fM, dA, Dh, jx, eX, afK, hy, j, v, asA, AO, q, J, mj, aT, kf, dz, jj, aPF, ae, iU, oV, r, en, hf, t, ik, ck, Ta, iM, c, bt, aJc, iW, oA, jh, hF, ad, av, btw, bj, d, je, et, ff, xs, Of, eZ, dN, iV, eU, pc, aS, y, iu, Tc); #c1(STAT5A) c2(XP_005257681) c3(signal transducer and activator of transcription 5A isoform X1) c4( ) c5(275320, 275321, 275322, 275323, 275324, 275325) c6( ) c7(bM, T, if, fd, ci, amW, x, Ds, K, cp, zQ, aW, Ls, aGQ, g, eE, cm, bw, eM, iT, cj, k, m, in, Ro, lT, n, aj, qN, zP, bkU, kH, h, IG, eV, bl, ox, A, aq, Og, pm, yR, ok, dB, ln, pg, B, bL, Dw, fM, dA, Dh, jx, eX, afK, hy, j, v, asA, AO, q, J, mj, aT, kf, dz, jj, aPF, ae, iU, oV, r, en, hf, t, ik, ck, Ta, iM, c, bt, aJc, iW, oA, jh, hF, ad, av, btw, bj, d, je, et, ff, xs, Of, eZ, dN, iV, eU, pc, aS, y, iu, Tc); #c1(STAT5B) c2(NP_036580) c3(signal transducer and activator of transcription 5B) c4(174514) c5(174515, 174516, 174517, 174518, 174519, 174520, 174521) c6( ) c7(T, if, fd, ci, amW, x, aE, K, aa, aW, Ls, g, eE, cm, bw, eM, iT, cj, k, m, lT, aj, qN, bkU, kH, h, lG, By, eV, ox, A, Rj, aq, Og, pm, yR, dB, ln, Ei, pg, B, fM, Dh, eX, afK, hy, v, asA, avO, q, J, eA, mj, aT, kf, dz, jj, bq, ae, iU, oV, Y, r, hf, t, iK, ck, Ta, iM, bKn, bt, aJc, gV, Ve, iW, DA, jh, hF, ad, av, btw, bj, ji, jo, d, je, hG, et, Of, dN, aDw, eU, pc, aS, y, iu, Tc); #c1(STAT5B) c2(XP_005257683) c3(signal transducer and activator of transcription 5B isoform X2) c4(275326) c5(275327, 275328, 275329, 275330, 275331, 275332) c6( ) c7(T, if, fd, ci, amW, x, aE, K, aa, aW, Ls, g, eE, cm, bw, eM, iT, cj, k, m, lT, aj, qN, bkU, kH, h, lG, By, eV, ox, A, Rj, aq, Og, pm, yR, dB, ln, Ei, pg, B, fM, Dh, eX, afK, hy, v, asA, avO, q, J, eA, mj, aT, kf, dz, jj, bq, ae, iU, oV, Y, r, hf, t, ik, ck, Ta, iM, bKn, bt, aJc, gV, Ve, iW, oA, jh, hF, ad, av, btw, bj, ji, jo, d, je, hG, et, Of, dN, aDw, eU, pc, aS, y, iu, Tc); #c1(STAT6) c2(XP_006719637) c3(signal transducer and activator of transcription 6 isoform X1) c4(292755, 292756, 292757, 292758, 292759, 292760, 292761, 292762, 292763, 292764) c5(292765, 292766, 292767, 292768, 292769, 292770, 292771, 292772, 292773, 292774, 292775, 292776, 292777, 292778, 292779, 292780, 292781, 292782, 292783, 292784, 292785, 292786, 292787, 292788, 292789, 292790, 292791, 292792, 292793, 292794, 292795, 292796, 292797, 292798, 292799, 292800, 292801, 292802, 292803, 292804, 292805, 292806, 292807, 292808, 292809, 292810, 292811, 292812, 292813, 292814, 292815, 292816, 292817, 292818, 292819, 292820, 292821, 292822, 292823, 292824, 292825, 292826, 292827, 292828, 292829, 292830, 292831, 292832, 292833, 292834, 292835, 292836, 292837, 292838, 292839, 292840, 292841, 292842, 292843, 292844, 292845, 292846, 292847, 292848, 292849, 292850, 292851) c6( ) c7(bM, T, fR, ea, if, bB, ci, bv, dV, Ds, aW, Du, g, as, eE, eM, iA, blQ, cj, k, Hb, dD, akc, n, fr, qN, ai, cU, lG, eV, cml, Nr, sp, fe, aq, po, at, B, Dw, fM, eR, ex, hy, j, bX, V, q, eA, arH, apJ, aT, ae, r, akY, ck, cT, hr, wR, dy, ij, bt, aD, iW, jh, wV, ah, eq, ad, dP, av, hD, ji, lQ, d, bPY, eZ, dN, hp, Fq, iV, DF, Nq, bE, z, aFb, y, X); #c1(STAT6) c2(XP_011537009) c3(signal transducer and activator of transcription 6 isoform X2) c4(334751) c5( ) c6( ) c7(bM, T, FR, ea, if, bB, ci, bv, dV, Ds, aW, Du, g, as, eE, eM, iA, blQ, cj, k, Hb, dD, akc, n, fr, qN, ai, cu, lG, eV, cml, Nr, sp, fe, aq, po, at, B, Dw, fM, eR, ex, hy, j, bX, V, q, eA, arH, apJ, aT, ae, r, akY, ck, cT, hr, wR, dy, ij, bt, aD, iW, jh, wV, ah, eq, ad, dP, av, hD, ji, lQ, d, bPY, eZ, dN, hp, Fq, iV, DF, Nq, bE, z, aFb, y, X); #c1(STATH) c2(NP_001009181) c3(statherin isoform b precursor) c4(23510) c5(23511, 23512, 23513, 23514, 23515, 23516) c6( ) c7(bN); #c1(STATH) c2(NP_003145) c3(statherin isoform a precursor) c4(135679) c5(135680, 135681, 135682, 135683, 135684, 135685) c6( ) c7(bN); #c1(STAU2) c2(NP_001157852) c3(double-stranded RNA-binding protein Staufen homolog 2 isoform a) c4(62769) c5(62770, 62771, 62772, 62773, 62774, 62775) c6( ) c7(V, fq); #c1(STAU2) c2(NP_001157853) c3(double-stranded RNA-binding protein Staufen homolog 2 isoform b) c4(62776, 62777, 62778, 62779, 62780) c5(62781, 62782, 62783, 62784, 62785, 62786, 62787) c6( ) c7(V, fq); #c1(STAU2) c2(NP_001157854) c3(double-stranded RNA-binding protein Staufen homolog 2 isoform c) c4(62788) c5(62789, 62790, 62791, 62792, 62793, 62794) c6( ) c7(V, fq); #c1(STAU2) c2(NP_001157855) c3(double-stranded RNA-binding protein Staufen homolog 2 isoform d) c4(62795) c5(62796, 62797, 62798, 62799, 62800) c6( ) c7(V, fq); #c1(STAU2) c2(NP_001157856) c3(double-stranded RNA-binding protein Staufen homolog 2 isoform e) c4( ) c5(62801, 62802, 62803, 62804, 62805, 62806, 62807, 62808, 62809, 62810, 62811, 62812, 62813, 62814, 62815, 62816, 62817, 62818, 62819, 62820, 62821, 62822, 62823, 62824, 62825, 62826, 62827, 62828, 62829) c6( ) c7(V, fq); #c1(STAU2) c2(NP_001157857) c3(double-stranded RNA-binding protein Staufen homolog 2 isoform f) c4(62830) c5(62831, 62832, 62833, 62834, 62835) c6( ) c7(V, fq); #c1(STBD1) c2(NP_003934) c3(starch-binding domain-containing protein 1) c4(142296, 142297) c5(142298, 142299, 142300, 142301, 142302, 142303, 142304, 142305, 142306, 142307, 142308, 142309) c6( ) c7(ex, sT, an); #c1(STC1) c2(NP_003146) c3(stanniocalcin-1 precursor) c4(135686, 135687) c5(135688, 135689, 135690, 135691, 135692, 135693, 135694, 135695, 135696, 135697, 135698, 135699, 135700, 135701, 135702, 135703, 135704, 135705, 135706, 135707, 135708) c6(135709) c7(aLC, T, jv, bkx, if, aA, bB, qN, AO, q, ad, aJf, mj, aT, bUP, lG, K, aDW, ae, Yi, azh, aa, eu, bbC, aW, Hq, r, aF, cl, t, nC, fq, bz, cma, ck, W, nb, c, bE, eM, bP, au, X, Ji, gX, hl, nw, dy, m, it, B, de); #c1(STC2) c2(NP_003705) c3 (stanniocalcin-2 precursor) c4(140280) c5(140281, 140282, 140283, 140284, 140285, 140286, 140287, 140288) c6(140289, 140290) c7(T, ht, aA, zP, q, ad, dP, hb, aT, lG, bta, K, hu, ae, aW, vj, r, aF, cm, t, ar, bP, c, wl, m, y, it); #c1(STEAP1) c2(NP_036581) c3(metalloreductase STEAP1) c4(174522) c5(174523, 174524, 174525, 174526, 174527, 174528) c6( ) c7(T, eM, aW, aT, n, v, np, K, m, t, ae, dx, ad); #c1(STEAP2) c2(NP_001231873) c3(metalloreductase STEAP2 isoform a) c4(87592) c5(87593, 87594, 87595, 87596, 87597, 87598, 87599, 87600, 87601, 87602, 87603, 87604, 87605, 87606) c6( ) c7(ae, ci, T, dx, ad); #c1(STEAP2) c2(NP_001231874) c3(metalloreductase STEAP2 isoform b) c4(87607) c5(87608, 87609, 87610, 87611, 87612, 87613, 87614, 87615, 87616, 87617, 87618, 87619, 87620, 87621) c6( ) c7(ae, ci, T, dx, ad); #c1(STEAP2) c2(NP_001231875) c3(metalloreductase STEAP2 isoform c) c4(87622, 87623, 87624) c5(87625, 87626, 87627, 87628, 87629, 87630, 87631, 87632, 87633, 87634, 87635, 87636, 87637, 87638, 87639, 87640, 87641, 87642) c6( ) c7(ae, ci, T, dx, ad); #c1(STEAP3) c2(NP_001008410) c3(metalloreductase STEAP3 isoform b) c4(23191) c5(23192, 23193, 23194, 23195, 23196, 23197, 23198, 23199, 23200, 23201, 23202, 23203, 23204, 23205, 23206, 23207, 23208, 23209, 23210, 23211, 23212, 23213, 23214) c6( ) c7(ae, gk, T, cJm, aNv, al, r, bGZ, K, ci, avG, Rc, bGO, q, ad); #c1(STEAP3) c2(NP_878919) c3(metalloreductase STEAP3 isoform a) c4(257213, 257214) c5(257215, 257216, 257217, 257218, 257219, 257220, 257221, 257222, 257223, 257224, 257225) c6( ) c7(ae, gk, T, cJm, aNv, al, r, bGZ, K, ci, avG, Rc, bGO, q, ad); #c1(STEAP4) c2(NP_001192245) c3(metalloreductase STEAP4 isoform 2) c4(82135) c5(82136, 82137, 82138, 82139, 82140, 82141) c6( ) c7(bL, dD, ea, v, eq, ei, qO, ae, ad); #c1(STEAP4) c2(NP_078912) c3(metalloreductase STEAP4 isoform 1) c4(216088) c5(216089, 216090, 216091, 216092, 216093, 216094) c6( ) c7(bL, dD, ea, v, eq, ei, qO, ae, ad); #c1(STH) c2(NP_001007533) c3(saitohin) c4(22774) c5(22775, 22776, 22777, 22778, 22779, 22780) c6( ) c7(Hx, rR, bb, WY, U, act, an, af); #c1(STIL) c2(XP_011540294) c3(SCL-interrupting locus protein isoform X2) c4(338688) c5(338689, 338690, 338691, 338692, 338693, 338694, 338695) c6( ) c7(T, if, DV, cJn, ci, nA, qN, x, fx, akU, h, aij, aii, ff, kp, en, g, QK, ck, dc, cj, Lo, k, lC); #c1(STIL) c2(XP_011540295) c3(SCL-interrupting locus protein isoform X1) c4(338696) c5(338697, 338698, 338699, 338700, 338701, 338702, 338703, 338704, 338705, 338706, 338707, 338708, 338709, 338710) c6( ) c7(T, if, DV, cJn, ci, nA, qN, x, fx, akU, h, aij, aii, ff, kp, en, g, QK, ck, dc, cj, Lo, k, lC); #c1(STIL) c2(XP_011540300) c3(SCL-interrupting locus protein isoform X5) c4(338711) c5( ) c6( ) c7(T, if, DV, cJn, ci, nA, qN, x, fx, akU, h, aij, aii, ff, kp, en, g, QK, ck, dc, cj, Lo, k, lC); #c1(STIL) c2(XP_011540302) c3(SCL-interrupting locus protein isoform X6) c4( ) c5(338712) c6( ) c7(T, if, DV, cJn, ci, nA, qN, x, fx, akU, h, aij, aii, ff, kp, en, g, QK, ck, dc, cj, Lo, k, lC); #c1(STIL) c2(XP_011540303) c3(SCL-interrupting locus protein isoform X7) c4( ) c5(338713, 338714, 338715, 338716, 338717) c6( ) c7(T, if, DV, cJn, ci, nA, qN, x, fx, akU, h, aij, aii, ff, kp, en, g, QK, ck, dc, cj, Lo, k, lC); #c1(STIM1) c2(NP_003147) c3(stromal interaction molecule 1 isoform 2 precursor) c4(135710) c5(135711, 135712, 135713, 135714, 135715, 135716) c6( ) c7(bM, T, dD, hl, aik, Dh, ea, if, Or, iB, cJo, cz, n, eq, ci, qN, mC, er, eA, bv, bjv, dV, aT, h, cJp, af, aa, d, mR, aW, Y, Ls, ff, eZ, dN, lo, cnc, gm, beP, en, cl, t, cm, bz, Qt, dr, nM, eM, dB, AM, gX, hL, gZ, dX, cS, m, fl, bt, B); #c1(STIM2) c2(NP_001162588) c3(stromal interaction molecule 2 isoform 3 precursor) c4(66406, 66407) c5(66408, 66409, 66410, 66411, 66412, 66413, 66414, 66415, 66416, 66417, 66418, 66419) c6( ) c7(d, aik, bE, aW, Y, aT, r, bB, pg, j, aXL, cm, q); #c1(STIM2) c2(NP 001162589) c3(stromal interaction molecule 2 isoform 1 precursor) c4(66420, 66421) c5(66422, 66423, 66424, 66425, 66426, 66427, 66428, 66429, 66430, 66431, 66432, 66433, 66434) c6( ) c7(d, aik, bE, aW, Y, aT, r, bB, pg, j, aXL, cm, q); #c1(STIM2) c2(NP_065911) c3(stromal interaction molecule 2 isoform 2 precursor) c4(206183, 206184) c5(206185, 206186, 206187, 206188, 206189, 206190, 206191, 206192, 206193, 206194, 206195) c6( ) c7(d, aik, bE, aW, Y, aT, r, bB, pg, j, aXL, cm, q); #c1(STIP1) c2(NP_006810) c3(stress-induced-phosphoprotein 1 isoform b) c4(168292) c5(168293, 168294, 168295, 168296, 168297, 168298) c6( ) c7(nJ, bM, T, ft, d, Y, ff, U, aT, it, K, m, EY, f, t, en, bz, eA); #c1(STK10) c2(NP_005981) c3(serine/threonine-protein kinase 10) c4(160960) c5(160961, 160962, 160963, 160964, 160965, 160966, 160967, 160968, 160969, 160970) c6( ) c7(dN, Ld, lQ); #c1(STK11) c2(NP_000446) c3(serine/threonine-protein kinase STK11) c4(9078, 9079) c5(9080, 9081, 9082, 9083, 9084, 9085, 9086, 9087, 9088, 9089, 9090, 9091) c6( ) c7(T, aC, azf, if, fd, bB, ci, aE, K, hu, eL, cp, iE, aW, nY, g, cm, Qt, eM, go, avo, m, asW, ht, n, aj, qN, cc, KN, jy, dL, uV, ZN, YI, Yd, aq, kp, Ld, Ye, By, hk, JQ, mg, hl, at, HB, YH, B, bL, yk, aA, cz, v, q, J, eA, eJ, bGn, aT, dz, af, qw, kl, s, NE, Y, aEk, Lf, r, en, bu, cl, t, apx, ik, sS, nM, Dy, Li, YW, gX, np, bt, Gl, hl, aZ, dG, av, ccJ, py, bj, mu, aeQ, BO, ff, dq, aF, KM, bz, dr, bE, ei, MC, iu); #c1(STK11) c2(XP_011526511) c3(serine/threonine-protein kinase STK11 isoform X2) c4( ) c5(320808, 320809, 320810, 320811, 320812) c6( ) c7(T, aC, azf, if, fd, bB, ci, aE, K, hu, eL, cp, iE, aW, nY, g, cm, Qt, eM, go, avo, m, asW, ht, n, aj, qN, cc, KN, jy, dL, uV, ZN, YI, Yd, aq, kp, Ld, Ye, By, hk, JA, mg, hL, at, HB, YH, B, bL, yk, aA, cz, v, q, J, eA, eJ, bGn, aT, dz, af, qw, kl, s, NE, Y, aEk, Lf, r, en, bu, cl, t, apx, iK, sS, nM, Dy, Li, YW, gX, np, bt, Gl, hl, aZ, dG, av, ccJ, py, bj, mu, aeQ, BO, ff, dq, aF, KM, bz, dr, bE, ei, MC, iu); #c1(STK11IP) c2(NP_443134) c3(serine/threonine-protein kinase 11-interacting protein) c4(229598) c5(229599, 229600, 229601, 229602, 229603, 229604) c6( ) c7(g, MC); #c1(STK11IP) c2(XP_011508831) c3(serine/threonine-protein kinase fl-interacting protein isoform X5) c4(299418) c5(299419, 299420, 299421, 299422, 299423, 299424) c6( ) c7(g, MC); #c1(STK17A) c2(NP_004751) c3(serine/threonine-protein kinase 17A) c4(149263) c5(149264, 149265, 149266, 149267, 149268, 149269) c6( ) c7(T, cF, d, eD, Lf, K, bu, eA); #c1(STK17B) c2(NP_004217) c3(serine/threonine-protein kinase 17B) c4(144367, 144368) c5(144369, 144370, 144371, 144372, 144373, 144374, 144375, 144376, 144377, 144378, 144379, 144380, 144381, 144382, 144383, 144384) c6( ) c7(LB, T, hd, nM, r, Ea, cm, q); #c1(STK19) c2(NP_004188) c3(serine/threonine-protein kinase 19 isoform 1) c4(144177, 144178, 144179, 144180, 144181) c5(144182, 144183, 144184, 144185, 144186, 144187, 144188, 144189, 144190, 144191, 144192, 144193, 144194, 144195, 144196, 144197, 144198, 144199, 144200, 144201, 144202, 144203, 144204, 144205, 144206, 144207, 144208, 144209, 144210, 144211, 144212, 144213, 144214, 144215, 144216, 144217, 144218, 144219, 144220, 144221, 144222, 144223, 144224, 144225, 144226, 144227, 144228, 144229, 144230, 144231, 144232, 144233) c6( ) c7(bF, T, A, xX, lR, W, eZ, r, h, nw, nk, nC, q); #c1(STK19) c2(NP_115830) c3(serine/threonine-protein kinase 19 isoform 2) c4(224187, 224188) c5(224189, 224190, 224191, 224192, 224193, 224194, 224195, 224196, 224197, 224198, 224199, 224200, 224201, 224202, 224203, 224204, 224205, 224206, 224207, 224208, 224209, 224210, 224211, 224212) c6( ) c7(bF, T, A, xX, lR, W, eZ, r, h, nw, nk, nC, q); #c1(STK24) c2(NP_001027467) c3(serine/threonine-protein kinase 24 isoform b precursor) c4(30229, 30230, 30231) c5(30232, 30233, 30234, 30235, 30236, 30237, 30238, 30239, 30240, 30241, 30242, 30243, 30244, 30245, 30246, 30247, 30248, 30249, 30250) c6( ) c7(LB, qa, adi, fe, cy, bK, K, EL, pd, ae, af, ad); #c1(STK24) c2(NP_001273578) c3(serine/threonine-protein kinase 24 isoform c precursor) c4(105352, 105353) c5(105354, 105355, 105356, 105357, 105358, 105359) c6( ) c7(LB, qa, adi, fe, cy, bK, K, EL, pd, ae, af, ad); #c1(STK24) c2(NP_003567) c3(serine/threonine-protein kinase 24 isoform a precursor) c4(139219) c5(139220, 139221, 139222, 139223, 139224, 139225) c6( ) c7(LB, qa, adi, fe, cy, bK, K, EL, pd, ae, af, ad); #c1(STK24) c2(XP_005254135) c3(serine/threonine-protein kinase 24 isoform X1) c4( ) c5(273005, 273006, 273007, 273008, 273009) c6( ) c7(LB, qa, adi, fe, cy, bK, K, EL, pd, ae, af, ad); #c1(STK25) c2(NP_001258907) c3(serine/threonine-protein kinase 25 isoform 1) c4(96774, 96775) c5(96776, 96777, 96778, 96779, 96780, 96781, 96782, 96783, 96784, 96785, 96786, 96787, 96788, 96789, 96790, 96791, 96792) c6( ) c7(aW, cJq, aT, cy, K, EL, cm, ae, EJ); #c1(STK25) c2(NP_001269237) c3(serine/threonine-protein kinase 25 isoform 3) c4( ) c5(100836, 100837, 100838, 100839, 100840, 100841, 100842, 100843, 100844, 100845, 100846, 100847, 100848) c6( ) c7(aW, cJq, aT, cy, K, EL, cm, ae, EJ); #c1(STK25) c2(XP_006712260) c3(serine/threonine-protein kinase 25 isoform X3) c4(286586) c5(286587) c6( ) c7(aW, cJq, aT, cy, K, EL, cm, ae, EJ); #c1(STK25) c2(XP_011508797) c3(serine/threonine-protein kinase 25 isoform X1) c4(299394) c5( ) c6( ) c7(aW, cJq, aT, cy, K, EL, cm, ae, EJ); #c1(STK25) c2(XP_011508798) c3(serine/threonine-protein kinase 25 isoform X4) c4( ) c5(299395, 299396, 299397, 299398, 299399, 299400) c6( ) c7(aW, cJq, aT, cy, K, EL, cm, ae, EJ); #c1(STK26) c2(NP_001035917) c3(serine/threonine-protein kinase 26 isoform 3) c4(33376) c5(33377, 33378, 33379, 33380, 33381, 33382) c6( ) c7(T, n, ci, ae, B, ad); #c1(STK26) c2(NP_001035918) c3(serine/threonine-protein kinase 26 isoform 2) c4(33383) c5(33384, 33385, 33386, 33387, 33388, 33389) c6( ) c7(T, n, ci, ae, B, ad); #c1(STK26) c2(NP_057626) c3(serine/threonine-protein kinase 26 isoform 1) c4(191933) c5(191934, 191935, 191936, 191937, 191938, 191939) c6( ) c7(T, n, ci, ae, B, ad); #c1(STK3) c2(NP_006272) c3(serine/threonine-protein kinase 3 isoform 1) c4(163469) c5(163470, 163471, 163472, 163473, 163474, 163475) c6( ) c7(LB, hd, nB, qb, aW, r, aT, q, B); #c1(STK3) c2(XP_011515550) c3(serine/threonine-protein kinase 3 isoform X1) c4(308387) c5(308388, 308389, 308390, 308391, 308392, 308393) c6( ) c7(LB, hd, nB, qb, aW, r, al, q, B); #c1(STK31) c2(NP_001247433) c3(serine/threonine-protein kinase 31 isoform b) c4( ) c5(93601, 93602, 93603, 93604, 93605, 93606, 93607, 93608) c6( ) c7(T, bE, aw, ht, gX, r, bB, bu, bX, hu, q); #c1(STK31) c2(NP_001247434) c3(serine/threonine-protein kinase 31 isoform c) c4(93609) c5(93610, 93611, 93612, 93613, 93614, 93615) c6( ) c7(T, bE, aw, ht, gX, r, bB, bu, bX, hu, q); #c1(STK31) c2(NP_113602) c3(serine/threonine-protein kinase 31 isoform a) c4(221657, 221658) c5(221659, 221660, 221661, 221662, 221663, 221664, 221665, 221666) c6( ) c7(T, bE, aw, ht, gX, r, bB, bu, bX, hu, q); #c1(STK31) c2(XP_006715821) c3(serine/threonine-protein kinase 31 isoform X5) c4( ) c5(289819, 289820, 289821, 289822, 289823, 289824, 289825) c6( ) c7(T, bE, aw, ht, gX, r, bB, bu, bX, hu, q); #c1(STK31) c2(XP_011513751) c3(serine/threonine-protein kinase 31 isoform X2) c4( ) c5(306099, 306100, 306101, 306102, 306103) c6( ) c7(T, bE, aw, ht, gX, r, bB, bu, bX, hu, q); #c1(STK31) c2(XP_011513753) c3(serine/threonine-protein kinase 31 isoform X3) c4(306104) c5( ) c6( ) c7(T, bE, aw, ht, gX, r, bB, bu, bX, hu, q); #c1(STK31) c2(XP_011513754) c3(serine/threonine-protein kinase 31 isoform X4) c4(306105) c5( ) c6( ) c7(T, bE, aw, ht, gX, r, bB, bu, bX, hu, q); #c1(STK32A) c2(NP_001106195) c3(serine/threonine-protein kinase 32A isoform 1) c4(42383) c5(42384, 42385, 42386, 42387, 42388, 42389) c6( ) c7(bz, cd, hp); #c1(STK32A) c2(NP_001274669) c3(serine/threonine-protein kinase 32A isoform 3) c4(106609) c5( ) c6( ) c7(bz, cd, hp); #c1(STK32A) c2(NP_659438) c3(serine/threonine-protein kinase 32A isoform 2) c4(239902) c5(239903, 239904, 239905, 239906, 239907, 239908, 239909, 239910, 239911, 239912, 239913, 239914) c6( ) c7(bz, cd, hp); #c1(STK32B) c2(NP_060871) c3(serine/threonine-protein kinase 32B) c4(198206, 198207) c5(198208, 198209, 198210, 198211, 198212, 198213, 198214) c6( ) c7(Na, T, apM, EQ, Nc, boJ); #c1(STK32C) c2(NP_775846) c3(serine/threonine-protein kinase 32C) c4(250485, 250486) c5(250487, 250488, 250489, 250490, 250491, 250492) c6( ) c7(bL); #c1(STK32C) c2(XP_011537990) c3(serine/threonine-protein kinase 32C isoform X1) c4( ) c5(335528) c6( ) c7(bL); #c1(STK32C) c2(XP_011537991) c3(serine/threonine-protein kinase 32C isoform X2) c4(335529, 335530, 335531, 335532) c5(335533, 335534, 335535, 335536, 335537, 335538, 335539, 335540, 335541, 335542, 335543, 335544, 335545, 335546, 335547, 335548, 335549, 335550, 335551, 335552, 335553, 335554, 335555, 335556) c6( ) c7(bL); #c1(STK32C) c2(XP_011537998) c3(serine/threonine-protein kinase 32C isoform X5) c4( ) c5(335557, 335558, 335559, 335560, 335561) c6( ) c7(bL); #c1(STK33) c2(NP_001275990) c3(serine/threonine-protein kinase 33 isoform a) c4(107659, 107660, 107661, 107662) c5(107663, 107664, 107665, 107666, 107667, 107668, 107669, 107670, 107671, 107672, 107673, 107674, 107675, 107676) c6( ) c7(bL, bM, T, fq, v); #c1(STK33) c2(XP_011518600) c3(serine/threonine-protein kinase 33 isoform X3) c4(312747, 312748) c5(312749, 312750, 312751, 312752, 312753, 312754, 312755, 312756, 312757, 312758, 312759, 312760, 312761, 312762, 312763, 312764, 312765, 312766, 312767, 312768, 312769, 312770, 312771, 312772, 312773, 312774, 312775, 312776, 312777, 312778) c6( ) c7(bL, bM, T, fq, v); #c1(STK35) c2(NP_543026) c3(serine/threonine-protein kinase 35) c4(233280, 233281) c5(233282, 233283, 233284, 233285, 233286, 233287, 233288, 233289, 233290, 233291, 233292, 233293) c6( ) c7(j); #c1(STK35) c2(XP_011527476) c3(serine/threonine-protein kinase 35 isoform X1) c4(322237) c5(322238, 322239, 322240, 322241, 322242, 322243) c6( ) c7(j); #c1(STK38L) c2(NP_055815) c3(serine/threonine-protein kinase 38-like) c4(183674, 183675, 183676) c5(183677, 183678, 183679, 183680, 183681, 183682, 183683, 183684, 183685, 183686, 183687, 183688, 183689, 183690, 183691, 183692, 183693, 183694, 183695, 183696, 183697, 183698, 183699, 183700, 183701, 183702) c6( ) c7(hk, T, nw); #c1(STK38L) c2(XP_005253399) c3(serine/threonine-protein kinase 38-like isoform X2) c4(272464) c5(272465) c6( ) c7(hk, T, nw); #c1(STK38L) c2(XP_011518915) c3(serine/threonine-protein kinase 38-like isoform X4) c4( ) c5(313339, 313340, 313341, 313342, 313343) c6( ) c7(hk, T, nw); #c1(STK39) c2(NP_037365) c3(STE20/SPS1-related proline-alanine-rich protein kinase) c4(175037) c5(175038, 175039, 175040, 175041, 175042, 175043) c6( ) c7(qa, bN, fM, if, oe, aT, cz, aj, v, afV, K, qN, an, ae, J, ad); #c1(STK4) c2(NP_006273) c3(serine/threonine-protein kinase 4) c4(163476, 163477) c5(163478, 163479, 163480, 163481, 163482, 163483, 163484, 163485, 163486, 163487, 163488, 163489) c6( ) c7(T, bwU, bgQ, aw, aA, bB, ci, V, q, ad, hd, cc, aE, aT, K, ae, d, aW, Y, r, fe, aF, cl, iV, cm, nM, eM, nB, bE, mx, hL, apv, ra, zA, B); #c1(STK4) c2(XP_005260590) c3(serine/threonine-protein kinase 4 isoform X4) c4(276729) c5(276730, 276731, 276732, 276733, 276734, 276735, 276736, 276737, 276738, 276739) c6( ) c7(T, bwU, bgQ, aw, aA, bB, ci, V, q, ad, hd, cc, aE, aT, K, ae, d, aW, Y, r, fe, aF, cl, iV, cm, nM, eM, nB, bE, mx, hl, apv, ra, zA, B); #c1(STMN2) c2(NP_001186143) c3(stathmin-2 isoform 1) c4(77211) c5(77212, 77213, 77214, 77215, 77216, 77217) c6( ) c7(cZ, aaF, F, dt, aqJ, K, jX, dd, ahl, H, dS, ahE, L); #c1(STMN2) c2(NP_008960) c3(stathmin-2 isoform 2) c4(169917, 169918) c5(169919, 169920, 169921, 169922, 169923, 169924, 169925, 169926, 169927, 169928, 169929, 169930) c6( ) c7(cZ, aaF, F, dt, aqJ, K, jX, dd, ahl, H, dS, ahE, L); #c1(STMN3) c2(NP_001263239) c3(stathmin-3 isoform 2) c4( ) c5(97082, 97083, 97084, 97085, 97086) c6( ) c7(BM, aW, ff, NT, aT, g, en); #c1(STMN3) c2(NP_056978) c3(stathmin-3 isoform 1) c4(188310, 188311) c5(188312, 188313, 188314, 188315, 188316, 188317, 188318) c6( ) c7(eM, aW, ff, NT, aT, g, en); #c1(STMN4) c2(NP_001269983) c3(stathmin-4 isoform 3) c4(103355) c5(103356, 103357, 103358, 103359, 103360, 103361) c6( ) c7(cm); #c1(STMN4) c2(NP_110422) c3(stathmin-4 isoform 1) c4(220256) c5(220257, 220258, 220259, 220260, 220261, 220262) c6( ) c7(cm); #c1(STOM) c2(NP_004090) c3(erythrocyte band 7 integral membrane protein isoform a) c4(143483, 143484) c5(143485, 143486, 143487, 143488, 143489, 143490, 143491, 143492, 143493, 143494, 143495, 143496) c6( ) c7(Na, T, Fd, eM, hQ, Nc, cfR, czp, xw, it, eA); #c1(STOM) c2(NP_937837) c3(erythrocyte band 7 integral membrane protein isoform b) c4(259258) c5(259259, 259260, 259261, 259262, 259263, 259264) c6( ) c7(Na, T, Fd, eM, hl, Nc, cfR, czp, xw, it, eA); #c1(STOML1) c2(NP_001243602) c3(stomatin-like protein 1 isoform 3) c4(91140) c5(91141, 91142, 91143, 91144, 91145, 91146) c6( ) c7(h); #c1(STOML1) c2(NP_001243603) c3(stomatin-like protein 1 isoform 4) c4(91147) c5(91148, 91149, 91150, 91151, 91152, 91153) c6( ) c7(h); #c1(STOML1) c2(NP_001243604) c3(stomatin-like protein 1 isoform 5) c4(91154) c5(91155, 91156, 91157, 91158, 91159, 91160) c6( ) c7(h); #c1(STOML1) c2(NP_001243605) c3(stomatin-like protein 1 isoform 6) c4(91161) c5(91162, 91163, 91164, 91165, 91166) c6( ) c7(h); #c1(STOML1) c2(NP_001243606) c3(stomatin-like protein 1 isoform 7) c4(91167) c5(91168, 91169, 91170, 91171, 91172, 91173, 91174) c6( ) c7(h); #c1(STOML1) c2(NP_004800) c3(stomatin-like protein 1 isoform 1) c4(149959) c5(149960, 149961, 149962, 149963, 149964, 149965) c6( ) c7(h); #c1(STOML2) c2(NP_001273960) c3(stomatin-like protein 2, mitochondrial isoform b) c4(105937) c5(105938, 105939, 105940, 105941, 105942, 105943) c6( ) c7(zJ, T, AM, ff, Of, aA, io, it, fd, aF, Og, K, en, hB, g, bz, eA, cnb); #c1(STOML2) c2(NP_038470) c3(stomatin-like protein 2, mitochondrial isoform a) c4(176578) c5(176579, 176580, 176581, 176582, 176583, 176584) c6( ) c7(zJ, T, AM, ff, Of, aA, io, it, fd, aF, Og, K, en, hB, g, bz, eA, cnb); #c1(STOML3) c2(NP_001137505) c3(stomatin-like protein 3 isoform 2) c4(55935) c5(55936, 55937, 55938, 55939, 55940) c6( ) c7(mj, agl, eA, nA, cfR, rc, On); #c1(STOML3) c2(NP_660329) c3(stomatin-like protein 3 isoform 1) c4(240949) c5(240950, 240951, 240952, 240953, 240954, 240955, 240956) c6( ) c7(mj, agl, eA, nA, cfR, rc, On); #c1(STON1) c2(NP_001185524) c3(stonin-1) c4(75775) c5(75776, 75777, 75778, 75779, 75780, 75781, 75782, 75783, 75784, 75785, 75786, 75787, 75788, 75789, 75790) c6( ) c7(q, BO, r); #c1(STON1-GTF2A1L) c2(NP_001185522) c3(STON1-GTF2A1L protein isoform 2) c4(75761) c5(75762, 75763, 75764, 75765, 75766, 75767) c6( ) c7(q, BO, r); #c1 (STON1-GTF2A1L) c2(NP_001185523) c3(STON1-GTF2A1L protein isoform 3) c4(75768) c5(75769, 75770, 75771, 75772, 75773, 75774) c6( ) c7(q, BO, r); #c1 (STON1-GTF2A1L) c2(NP_758515) c3(STON1-GTF2A1L protein isoform 1) c4(249224) c5(249225, 249226, 249227, 249228, 249229, 249230, 249231, 249232, 249233, 249234, 249235, 249236, 249237, 249238) c6( ) c7(q, BO, r); #c1(STOX1) c2(NP_001123631) c3(storkhead-box protein 1 isoform b) c4(47893) c5(47894, 47895, 47896, 47897, 47898, 47899) c6( ) c7(nJ, uC, eb, cz, cJr); #c1(STOX1) c2(NP_001123632) c3(storkhead-box protein 1 isoform c) c4(47900) c5(47901, 47902, 47903, 47904, 47905, 47906) c6( ) c7(nJ, uC, eb, cz, cJr); #c1(STOX1) c2(NP_001123633) c3(storkhead-box protein 1 isoform a) c4(47907) c5(47908, 47909, 47910, 47911, 47912, 47913, 47914, 47915, 47916, 47917, 47918, 47919, 47920, 47921, 47922, 47923) c6( ) c7(nJ, uC, eb, cz, cJr); #c1(STOX1) c2(XP_011537756) c3(storkhead-box protein 1 isoform X1) c4(335293) c5( ) c6( ) c7(nJ, uC, eb, cz, cJr); #c1(STRA13) c2(NP_001257935) c3(centromere protein X isoform 1) c4(95821) c5(95822, 95823, 95824, 95825, 95826, 95827) c6( ) c7(bL, T, py, eM, Ae, aW, aT, aA, oZ, aF, K); #c1(STRA13) c2(NP_659435) c3(centromere protein X isoform 2) c4(239881) c5(239882, 239883, 239884, 239885, 239886, 239887) c6( ) c7(bL, T, py, eM, Ae, aW, aT, aA, oZ, aF, K); #c1(STRA6) c2(NP_001136091) c3 (stimulated by retinoic acid gene 6 protein homolog isoform b) c4(54302) c5(54303, 54304, 54305, 54306, 54307) c6( ) c7(md, ctm, T, bL, kn, kj, bE, wk, X, Az, r, gZ, nA, od, bD, y, q, Gc, aa); #c1(STRA6) c2(NP_001136092) c3(stimulated by retinoic acid gene 6 protein homolog isoform c) c4(54308) c5(54309, 54310, 54311, 54312, 54313, 54314) c6( ) c7(md, ctm, T, bL, kn, kj, bE, wk, X, Az, r, gZ, nA, od, bD, y, q, Gc, aa); #c1(STRA6) c2(NP_001185969) c3(stimulated by retinoic acid gene 6 protein homolog isoform d) c4(76846) c5(76847, 76848, 76849, 76850, 76851, 76852, 76853, 76854, 76855) c6( ) c7(md, ctm, T, bL, kn, kj, bE, wk, X, Az, r, gZ, nA, oQ, bD, y, q, Gc, aa); #c1(STRA6) c2(NP_001185970) c3(stimulated by retinoic acid gene 6 protein homolog isoform e) c4(76856, 76857) c5( ) c6( ) c7(md, ctm, T, bL, kn, kj, bE, wk, X, Az, r, gZ, nA, ol, bD, y, q, Gc, aa); #c1 (STRA6) c2(NP_001185971) c3(stimulated by retinoic acid gene 6 protein homolog isoform f) c4(76858) c5(76859, 76860, 76861, 76862, 76863, 76864, 76865, 76866, 76867, 76868) c6( ) c7(md, ctm, T, bL, kn, kj, bE, wk, X, Az, r, gZ, nA, ol, bD, y, q, Gc, aa); #c1(STRA6) c2(XP_011520185) c3(stimulated by retinoic acid gene 6 protein homolog isoform X1) c4(315049) c5(315050, 315051, 315052, 315053, 315054, 315055, 315056, 315057, 315058, 315059, 315060, 315061, 315062, 315063) c6( ) c7(md, ctm, T, bL, kn, kj, bE, wk, X, Az, r, gZ, nA, od, bD, y, q, Gc, aa); #c1(STRA6) c2(XP_011520186) c3 (stimulated by retinoic acid gene 6 protein homolog isoform X2) c4(315064, 315065) c5(315066, 315067, 315068, 315069, 315070, 315071, 315072, 315073, 315074, 315075, 315076, 315077, 315078, 315079, 315080) c6( ) c7(md, ctm, T, bL, kn, kj, bE, wk, X, Az, r, gZ, nA, od, bD, y, q, Gc, aa); #c1(STRA8) c2(NP_872295) c3(stimulated by retinoic acid gene 8 protein homolog) c4(256037) c5(256038, 256039, 256040, 256041, 256042, 256043) c6( ) c7(vH, ND); #c1(STRADA) c2(NP_001003787) c3(STE20-related kinase adapter protein alpha isoform 1) c4(20093) c5(20094, 20095, 20096, 20097, 20098, 20099) c6( ) c7(jf, T, ha, cJs, dz, g, MC); #c1(STRADA) c2(NP_001159441) c3(STE20-related kinase adapter protein alpha isoform 5) c4(64056) c5(64057, 64058, 64059, 64060, 64061) c6( ) c7(jf, T, ha, cJs, dz, g, MC); #c1(STRADA) c2(NP_001159442) c3(STE20-related kinase adapter protein alpha isoform 6) c4(64062) c5(64063, 64064, 64065, 64066, 64067) c6( ) c7(jf, T, ha, cJs, dz, g, MC); #c1(STRADA) c2(NP_699166) c3(STE20-related kinase adapter protein alpha isoform 3) c4(245785, 245786) c5(245787, 245788, 245789, 245790, 245791, 245792, 245793, 245794, 245795, 245796, 245797, 245798) c6( ) c7(jf, T, ha, cJs, dz, g, MC); #c1(STRADA) c2(XP_005257855) c3 (STE20-related kinase adapter protein alpha isoform X4) c4(275384) c5( ) c6( ) c7(jf, T, ha, cJs, dz, g, MC); #c1(STRADA) c2(XP_005257856) c3(STE20-related kinase adapter protein alpha isoform X1) c4( ) c5(275385, 275386, 275387, 275388, 275389) c6( ) c7(jf, T, ha, cJs, dz, g, MC); #c1(STRADA) c2(XP_011523768) c3(STE20-related kinase adapter protein alpha isoform X3) c4( ) c5(318265, 318266) c6( ) c7(jf, T, ha, cJs, dz, g, MC); #c1(STRADA) c2(XP_011523769) c3(STE20-related kinase adapter protein alpha isoform X5) c4( ) c5(318267) c6( ) c7(jf, T, ha, cJs, dz, g, MC); #c1(STRADB) c2(NP_001193793) c3(STE20-related kinase adapter protein beta isoform 2) c4(82620) c5(82621, 82622, 82623, 82624, 82625) c6( ) c7(c); #c1(STRADB) c2(NP_061041) c3(STE20-related kinase adapter protein beta isoform 1) c4(199104, 199105) c5(199106, 199107, 199108, 199109, 199110, 199111, 199112, 199113, 199114, 199115, 199116, 199117, 199118, 199119, 199120, 199121, 199122) c6( ) c7(c); #c1(STRAP) c2(NP_009109) c3(serine-threonine kinase receptor-associated protein) c4(170725, 170726) c5(170727, 170728, 170729, 170730, 170731, 170732, 170733, 170734, 170735, 170736, 170737, 170738, 170739, 170740, 170741, 170742) c6( ) c7(T, aW, Ak, kf, aT, r, ci, iu, dx); #c1(STRAP) c2(XP_005253351) c3(serine-threonine kinase receptor-associated protein isoform X1) c4(272436) c5(272437, 272438, 272439, 272440, 272441) c6( ) c7(T, aW, Ak, kf, aT, r, ci, iu, dx); #c1(STRC) c2(NP_714544) c3(stereocilin precursor) c4(246565, 246566) c5(246567, 246568, 246569, 246570, 246571, 246572, 246573, 246574, 246575, 246576, 246577, 246578, 246579, 246580, 246581, 246582, 246583, 246584, 246585, 246586, 246587, 246588, 246589, 246590, 246591, 246592, 246593, 246594, 246595) c6( ) c7(cJt, bCd, gU, alA); #c1(STRC) c2(XP_011519579) c3(stereocilin isoform X1) c4(314247, 314248) c5(314249, 314250, 314251, 314252, 314253) c6( ) c7(cJt, bCd, gU, alA); #c1(STRC) c2(XP_011519581) c3(stereocilin isoform X2) c4(314254) c5( ) c6( ) c7(cJt, bCd, gU, alA); #c1(STRN) c2(NP_003153) c3(striatin) c4(135738, 135739) c5(135740, 135741, 135742, 135743, 135744, 135745, 135746, 135747, 135748, 135749, 135750, 135751) c6( ) c7(ac, wK, aw, e, j, eu); #c1(STRN) c2(XP_005264576) c3(striatin isoform X2) c4(279101) c5(279102, 279103, 279104, 279105, 279106, 279107) c6( ) c7(ac, wK, aw, e, j, eu); #c1(STRN3) c2(NP_001077362) c3(striatin-3 isoform 1) c4(37752) c5(37753, 37754, 37755, 37756, 37757, 37758) c6( ) c7(ft, B, aFQ, cd, Pr, pb, bAf); #c1(STRN3) c2(NP_055389) c3(striatin-3 isoform 2) c4(180732) c5(180733, 180734, 180735, 180736, 180737, 180738) c6( ) c7(ft, B, aFQ, cd, Pr, pb, bAf); #c1(STRN4) c2(NP_001034966) c3(striatin-4 isoform 2) c4(32415) c5(32416, 32417, 32418, 32419, 32420, 32421, 32422) c6( ) c7(T, aE, cj); #c1(STRN4) c2(NP_037535) c3(striatin-4 isoform 1) c4(176084, 176085) c5(176086, 176087, 176088, 176089, 176090, 176091, 176092, 176093, 176094, 176095, 176096, 176097) c6( ) c7(T, aE, cj); #c1(STRN4) c2(XP_006723237) c3(striatin-4 isoform X5) c4( ) c5(295048, 295049, 295050, 295051, 295052) c6( ) c7(T, aE, cj); #c1(STRN4) c2(XP_011525179) c3(striatin-4 isoform X4) c4(319625) c5( ) c6( ) c7(T, aE, cj); #c1(STS) c2(NP_000342) c3(steryl-sulfatase precursor) c4(7424) c5(7425, 7426, 7427, 7428, 7429, 7430) c6( ) c7(T, ea, if, al, bB, ci, ip, Gt, aE, dV, K, aW, fc, g, as, cm, eM, m, hh, dD, QO, n, nA, pM, qN, eW, yn, ft, alu, jz, ahj, h, aPe, Dz, yi, AQ, hQ, aq, ajz, KE, Qy, bP, dB, hL, hR, el, ZY, zl, cJu, B, cn, Dw, cz, v, q, eJ, UE, hW, akU, aoA, aT, vH, yu, agV, zg, bq, ae, kl, s, ND, Y, r, lo, en, cl, t, cio, bN, rR, au, aZ, jh, btu, eq, ad, afb, dP, av, btD, buR, ds, nd, fn, cJv, lQ, hP, d, ff, bz, dr, gk, mO, bE, y, X); #c1(STT3A) c2(NP_001265432) c3(dolichyl-diphosphooligosaccharide-protein glycosyltransferase subunit STT3A isoform a) c4(98583) c5(98584, 98585, 98586, 98587, 98588, 98589, 98590, 98591, 98592, 98593) c6( ) c7(qJ, cJw, pa, lg, dv, h); #c1(STT3B) c2(NP_849193) c3(dolichyl-diphosphooligosaccharide-protein glycosyltransferase subunit STT3B) c4(254482) c5(254483, 254484, 254485, 254486, 254487, 254488) c6( ) c7(cJx, SL); #c1(STUB1) c2(NP_005852) c3(E3 ubiquitin-protein ligase cHIP isoform a) c4(159883) c5(159884, 159885, 159886, 159887, 159888, 159889) c6( ) c7(bEn, T, ky, anl, ajm, kB, aW, af, U, aT, r, bw, K, m, Tp, t, q, aR, UE); #c1(STX11) c2(NP_003755) c3(syntaxin-11) c4( ) c5(140652, 140653, 140654, 140655, 140656) c6( ) c7(cJy, T, EA, pz, bw, bt, zA); #c1(STX11) c2(XP_011534515) c3(syntaxin-11 isoform X2) c4(331009) c5(331010) c6( ) c7(cJy, T, EA, pz, bw, bt, zA); #c1(STX16) c2(NP_001001433) c3(syntaxin-16 isoform a) c4(18426, 18427, 18428, 18429, 18430) c5(18431, 18432, 18433, 18434, 18435, 18436, 18437, 18438, 18439, 18440, 18441, 18442, 18443) c6( ) c7(bWA, dg, xW, EL, EJ); #c1(STX16) c2(NP_001128244) c3(syntaxin-16 isoform c) c4(49753, 49754, 49755, 49756) c5(49757, 49758, 49759, 49760, 49761, 49762, 49763) c6( ) c7(bWA, dg, xW, EL, EJ); #c1(STX16) c2(NP_001128245) c3(syntaxin-16 isoform d) c4(49764, 49765, 49766, 49767) c5(49768, 49769, 49770, 49771, 49772, 49773, 49774, 49775, 49776, 49777, 49778, 49779, 49780, 49781, 49782, 49783, 49784, 49785) c6( ) c7(bWA, dg, xW, EL, EJ); #c1(STX16) c2(NP_001191797) c3(syntaxin-16 isoform e) c4( ) c5(81769, 81770, 81771, 81772, 81773, 81774, 81775, 81776, 81777, 81778, 81779, 81780, 81781, 81782, 81783, 81784, 81785, 81786, 81787, 81788, 81789, 81790, 81791, 81792, 81793, 81794, 81795, 81796, 81797, 81798, 81799, 81800, 81801, 81802, 81803, 81804, 81805) c6( ) c7(bWA, dg, xW, EL, EJ); #c1(STX16) c2(NP_003754) c3(syntaxin-16 isoform b) c4(140642) c5(140643, 140644, 140645, 140646, 140647, 140648, 140649, 140650, 140651) c6( ) c7(bWA, dg, xW, EL, EJ); #c1(STX17) c2(NP_060389) c3(syntaxin-17) c4(195755, 195756) c5(195757, 195758, 195759, 195760, 195761, 195762, 195763, 195764, 195765, 195766, 195767, 195768, 195769, 195770, 195771, 195772, 195773, 195774, 195775, 195776, 195777, 195778, 195779, 195780) c6( ) c7(hk, Y, OW, cJz, bdY); #c1(STX18) c2(NP_058626) c3(syntaxin-18) c4(192833) c5(192834, 192835, 192836, 192837, 192838, 192839) c6( ) c7(Na, aC, aW, oe, cz, aT, Nc, rz, cgj, ad); #c1(STX1A) c2(NP_001159375) c3(syntaxin-IA isoform 2) c4(63913) c5(63914, 63915, 63916, 63917, 63918, 63919, 63920, 63921, 63922, 63923, 63924, 63925) c6( ) c7(bL, bVk, eR, aQc, ha, aj, v, uu, BF, nA, J, aE, ql, K, af, s, akq, bN, YE, alC, Gx, mf, pJ); #c1(STX1A) c2(NP_004594) c3(syntaxin-1A isoform 1) c4(147997, 147998, 147999) c5(148000, 148001, 148002, 148003, 148004, 148005) c6( ) c7(bL, bVk, eR, aQc, ha, aj, v, uu, BF, nA, J, aE, ql, K, af, s, akq, bN, YE, alC, Gx, mf, pJ); #c1(STX1A) c2(XP_011514843) c3(syntaxin-IA isoform X1) c4( ) c5(307584, 307585, 307586, 307587, 307588, 307589) c6( ) c7(bL, bVk, eR, aQc, ha, aj, v, uu, BF, nA, J, aE, ql, K, af, s, akq, bN, YE, alC, Gx, mf, pJ); #c1(STX1B) c2(NP_443106) c3(syntaxin-IB) c4(229453) c5(229454, 229455, 229456, 229457, 229458, 229459) c6( ) c7(ha); #c1(STX2) c2(NP_001971) c3(syntaxin-2 isoform 1) c4(122746) c5(122747, 122748, 122749, 122750, 122751, 122752) c6( ) c7(LB, iV, gU, pA, ql, fG, ajd, Ni, fe, dy, K, Hb, BF, lm, pJ, aDP, aY); #c1(STX2) c2(XP_005253613) c3(syntaxin-2 isoform X1) c4(272592) c5(272593, 272594, 272595, 272596, 272597, 272598) c6( ) c7(LB, iV, gU, pA, ql, fG, ajd, Ni, fe, dy, K, Hb, BF, lm, pJ, aDP, aY); #c1(STX3) c2(NP_001171511) c3 (syntaxin-3 isoform 2) c4(70357, 70358) c5(70359, 70360, 70361, 70362, 70363, 70364, 70365, 70366, 70367, 70368, 70369, 70370, 70371, 70372, 70373, 70374, 70375, 70376, 70377, 70378, 70379, 70380, 70381, 70382, 70383) c6( ) c7(bpj); #c1(STX3) c2(XP_005274257) c3(syntaxin-3 isoform X3) c4(285026) c5(285027, 285028, 285029, 285030, 285031) c6( ) c7(bpj); #c1(STX3) c2(XP_011543521) c3(syntaxin-3 isoform X1) c4(342391, 342392, 342393) c5( ) c6( ) c7(bpj); #c1(STX3) c2(XP_011543523) c3(syntaxin-3 isoform X4) c4( ) c5(342394, 342395, 342396, 342397, 342398, 342399) c6( ) c7(bpj); #c1(STX4) c2(NP_001259025) c3(syntaxin-4 isoform 2) c4(96951) c5(96952, 96953, 96954, 96955, 96956, 96957) c6( ) c7(bL, aj, J, ad); #c1(STX4) c2(NP_004595) c3(syntaxin-4 isoform 3) c4(148006) c5(148007, 148008, 148009, 148010, 148011, 148012) c6( ) c7(bL, aj, J, ad); #c1(STX5) c2(NP_001231595) c3(syntaxin-5 isoform 2) c4(87318) c5(87319, 87320, 87321, 87322, 87323, 87324) c6( ) c7(lc); #c1(STX5) c2(XP_011543524) c3(syntaxin-5 isoform X1) c4(342400, 342401, 342402) c5(342403, 342404, 342405, 342406, 342407, 342408, 342409) c6( ) c7(lc); #c1(STX5) c2(XP_011543525) c3(syntaxin-5 isoform X2) c4( ) c5(342410, 342411, 342412, 342413, 342414, 342415) c6( ) c7(lc); #c1(STX5) c2(XP_011543526) c3(syntaxin-5 isoform X3) c4( ) c5(342416, 342417, 342418, 342419, 342420) c6( ) c7(lc); #c1(STX6) c2(NP_001273139) c3(syntaxin-6 isoform 2) c4( ) c5(104349, 104350, 104351, 104352, 104353) c6( ) c7(ey, act); #c1(STX6) c2(NP_005810) c3(syntaxin-6 isoform 1) c4(159624, 159625) c5(159626, 159627, 159628, 159629, 159630, 159631, 159632) c6( ) c7(By, act); #c1(STX8) c2(NP_004844) c3(syntaxin-8) c4(150225) c5(150226, 150227, 150228, 150229, 150230, 150231) c6( ) c7(xZ, Lq, af); #c1(STXBP1) c2(NP_001027392) c3(syntaxin-binding protein 1 isoform b) c4(30116) c5(30117, 30118, 30119, 30120, 30121, 30122) c6( ) c7(cJC, bN, aqa, yr, cJB, hb, dc, ha, cC, h, lm, K, nA, xw, af, cJA); #c1(STXBP1) c2(NP_003156) c3(syntaxin-binding protein 1 isoform a) c4(135752, 135753, 135754) c5(135755, 135756, 135757, 135758, 135759, 135760, 135761, 135762, 135763, 135764, 135765, 135766, 135767, 135768, 135769, 135770, 135771, 135772) c6( ) c7(cJC, bN, aqa, yr, cJB, hb, dc, ha, cC, h, lm, K, nA, xw, af, cJA); #c1(STXBP2) c2(NP_001120868) c3(syntaxin-binding protein 2 isoform b) c4(44872) c5(44873, 44874, 44875, 44876, 44877, 44878) c6( ) c7(pz, kQ, ZA, CJD, dX); #c1(STXBP2) c2(NP_001258963) c3(syntaxin-binding protein 2 isoform c) c4(96821, 96822) c5(96823, 96824, 96825, 96826, 96827, 96828, 96829, 96830, 96831, 96832, 96833, 96834) c6( ) c7(pz, kQ, zA, cJD, dX); #c1(STXBP2) c2(NP_008880) c3(syntaxin-binding protein 2 isoform a) c4(169476, 169477) c5(169478, 169479, 169480, 169481, 169482, 169483, 169484, 169485, 169486, 169487, 169488, 169489) c6( ) c7(pz, kQ, zA, cJD, dX); #c1(STXBP4) c2(NP_848604) c3(syntaxin-binding protein 4) c4(253942) c5(253943, 253944, 253945, 253946, 253947, 253948) c6( ) c7(bL, aW, fe, fq, aT); #c1(STXBP4) c2(XP_006721860) c3(syntaxin-binding protein 4 isoform X6) c4(294327) c5(294328, 294329, 294330, 294331, 294332, 294333, 294334, 294335, 294336, 294337) c6( ) c7(bL, aW, fe, fq, aT); #c1(STXBP5) c2(NP_001121187) c3(syntaxin-binding protein 5 isoform b) c4(45718, 45719) c5(45720, 45721, 45722, 45723, 45724, 45725) c6( ) c7(QL, cd, pA); #c1(STXBP5) c2(NP_640337) c3(syntaxin-binding protein 5 isoform a) c4(238444, 238445) c5(238446, 238447, 238448, 238449, 238450, 238451, 238452, 238453, 238454, 238455, 238456, 238457) c6( ) c7(QL, cd, pA); #c1(STXBP5) c2(XP_006715402) c3(syntaxin-binding protein 5 isoform X4) c4( ) c5(289523) c6( ) c7(QL, cd, pA); #c1(STXBP5) c2(XP_011533753) c3(syntaxin-binding protein 5 isoform X3) c4(329872, 329873) c5(329874, 329875, 329876, 329877, 329878, 329879, 329880, 329881, 329882, 329883, 329884, 329885) c6( ) c7(AL, cd, pA); #c1(STXBP5) c2(XP_011533755) c3(syntaxin-binding protein 5 isoform X6) c4(329886, 329887) c5(329888, 329889, 329890, 329891, 329892, 329893, 329894, 329895, 329896, 329897, 329898) c6( ) c7(QL, cd, pA); #c1(STXBP5) c2(XP_011533756) c3(syntaxin-binding protein 5 isoform X7) c4( ) c5(329899, 329900, 329901, 329902, 329903, 329904, 329905) c6( ) c7(QL, cd, pA); #c1(STXBP5L) c2(NP_055795) c3(syntaxin-binding protein 5-like) c4(183556) c5(183557, 183558, 183559, 183560, 183561, 183562) c6( ) c7(F, al); #c1(STXBP5L) c2(XP_011511635) c3(syntaxin-binding protein 5-like isoform X8) c4( ) c5(302391, 302392, 302393, 302394, 302395, 302396) c6( ) c7(F, al); #c1(STXBP6) c2(NP_001291406) c3(syntaxin-binding protein 6) c4(115890, 115891, 115892) c5(115893, 115894, 115895, 115896, 115897, 115898, 115899, 115900, 115901, 115902, 115903, 115904, 115905, 115906, 115907, 115908, 115909, 115910, 115911, 115912, 115913, 115914, 115915, 115916, 115917, 115918, 115919, 115920, 115921, 115922, 115923) c6( ) c7(y, De, cd, bN, PN); #c1(STXBP6) c2(XP_011534971) c3(syntaxin-binding protein 6 isoform X1) c4( ) c5(331526, 331527, 331528, 331529, 331530, 331531) c6( ) c7(y, oe, cd, bN, pN); #c1(STYK1) c2(XP_005253474) c3(tyrosine-protein kinase STYK1 isoform X1) c4(272518) c5(272519, 272520, 272521, 272522, 272523, 272524, 272525, 272526) c6(272527, 272528) c7(av, T, eM, aT, bz, bw, ae, ad); #c1(STYK1) c2(XP_011519041) c3(tyrosine-protein kinase STYK1 isoform X2) c4(313508, 313509, 313510, 313511) c5(313512, 313513, 313514, 313515, 313516, 313517, 313518, 313519, 313520, 313521, 313522, 313523, 313524, 313525, 313526, 313527, 313528, 313529, 313530, 313531, 313532, 313533, 313534, 313535) c6( ) c7(av, T, eM, aT, bz, bw, ae, ad); #c1(STYX) c2(NP_001124173) c3(serine/threonine/tyrosine-interacting protein) c4(48406) c5(48407, 48408, 48409, 48410, 48411, 48412, 48413, 48414, 48415, 48416) c6( ) c7(A, gU, ND, vH, aj, J); #c1(SUB1) c2(NP_006704) c3(activated RNA polymerase II transcriptional coactivator p15) c4(167261, 167262, 167263) c5(167264, 167265, 167266, 167267, 167268, 167269, 167270, 167271, 167272, 167273, 167274, 167275, 167276, 167277, 167278, 167279, 167280, 167281, 167282, 167283, 167284, 167285, 167286, 167287, 167288, 167289, 167290, 167291, 167292, 167293, 167294, 167295, 167296, 167297) c6( ) c7(sc, T, aC, fM, id, aEj, ht, jx, jh, aA, fd, j, ci, fr, hF, Fc, q, ad, eA, ip, x, av, iu, ft, fx, ib, nv, kH, aT, h, hn, dL, K, jj, XZ, hu, rz, hS, ae, eL, Zm, pq, jf, d, je, s, aix, iU, aW, Y, ff, r, aF, en, cl, g, t, as, iK, bw, iP, ck, qJ, iM, bP, eM, aJm, dB, iT, cj, pg, k, EY, bt, B, vi); #c1(SUCLA2) c2(NP_003841) c3(succinyl-CoA ligase [ADP-forming] subunit beta, mitochondrial precursor) c4(141280, 141281, 141282, 141283, 141284, 141285, 141286) c5(141287, 141288, 141289, 141290, 141291, 141292, 141293, 141294, 141295, 141296, 141297, 141298, 141299, 141300, 141301, 141302, 141303, 141304, 141305, 141306, 141307, 141308) c6( ) c7(dD, ea, ha, l, aj, bS, v, ct, eq, nE, aoz, J, eW, K, af, cJF, cmG, Z, f, px, SE, cJE, R, awL, L); #c1(SUCLA2) c2(XP_011533594) c3(succinyl-CoA ligase [ADP-forming] subunit beta, mitochondrial isoform X1) c4( ) c5(329678, 329679, 329680, 329681, 329682, 329683, 329684) c6( ) c7(dD, ea, ha, l, aj, bS, v, ct, eq, nE, aoz, J, eW, K, af, cJF, cmG, Z, f, px, SE, cJE, R, awl, L); #c1(SUCLA2) c2(XP_011533595) c3(succinyl-CoA ligase [ADP-forming] subunit beta, mitochondrial isoform X2) c4( ) c5(329685, 329686, 329687, 329688, 329689, 329690, 329691, 329692, 329693, 329694, 329695, 329696, 329697, 329698, 329699, 329700, 329701) c6( ) c7(dD, ea, ha, l, aj, bS, v, ct, eq, nE, aoz, J, eW, K, af, cJF, cmG, Z, f, px, SE, cJE, R, awL, L); #c1(SUCLG1) c2(NP_003840) c3(succinyl-CoA ligase [ADP/GDP-forming] subunit alpha, mitochondrial precursor) c4(141273) c5(141274, 141275, 141276, 141277, 141278, 141279) c6( ) c7(cJG, aoz, clb, aA); #c1(SUCLG2) c2(NP_001171070) c3(succinyl-CoA ligase [GDP-forming] subunit beta, mitochondrial isoform 1 precursor) c4(69803, 69804) c5(69805, 69806, 69807, 69808, 69809, 69810) c6( ) c7(dP, ayB, v, cF); #c1(SUCLG2) c2(NP_003839) c3(succinyl-CoA ligase [GDP-forming] subunit beta, mitochondrial isoform 2 precursor) c4(141260) c5(141261, 141262, 141263, 141264, 141265, 141266, 141267, 141268, 141269, 141270, 141271, 141272) c6( ) c7(dP, ayB, v, cF); #c1(SUCNR1) c2(NP_149039) c3(succinate receptor 1) c4(226889) c5(226890, 226891, 226892, 226893, 226894, 226895) c6( ) c7(cZ, dD, ac, W, ea, cz, yy, eq, dd, aeP, aY, vP); #c1(SUCO) c2(NP_055098) c3(SUN domain-containing ossification factor isoform 1 precursor) c4(178920) c5(178921, 178922, 178923, 178924, 178925, 178926) c6( ) c7(h, Ew, bNo); #c1(SUCO) c2(NP_057311) c3(SUN domain-containing ossification factor isoform 2) c4(190046) c5(190047, 190048, 190049, 190050, 190051, 190052) c6( ) c7(h, Ew, bNo); #c1(SUDS3) c2(NP_071936) c3(sin3 histone deacetylase corepressor complex component SDS3) c4(212396, 212397) c5(212398, 212399, 212400, 212401, 212402, 212403, 212404, 212405, 212406, 212407) c6( ) c7(aHP, T, aHQ, cz); #c1(SUDS3) c2(XP_011536973) c3(sin3 histone deacetylase corepressor complex component SDS3 isoform X1) c4( ) c5(334666, 334667) c6( ) c7(aHP, T, aHQ, cz); #c1(SUFU) c2(NP_001171604) c3(suppressor of fused homolog isoform 2) c4( ) c5(70733, 70734, 70735, 70736, 70737, 70738) c6( ) c7(jf, bv, asW, BN, Qr, asl, dB, Y, hy, C, YH, ne, ip, hS); #c1(SUFU) c2(NP_057253) c3(suppressor of fused homolog isoform 1) c4(189730, 189731) c5(189732, 189733, 189734, 189735, 189736, 189737) c6( ) c7(jf, bv, asW, BN, Qr, asl, dB, Y, hy, C, YH, ne, ip, hS); #c1(SUGCT) c2(NP_001180240) c3(succinate-hydroxymethylglutarate CoA-transferase isoform 1) c4(73497) c5(73498, 73499, 73500, 73501, 73502, 73503) c6( ) c7(aC, j, k, Ea, uu, cJH, fq, cd, ad); #c1(SUGCT) c2(NP_001180241) c3(succinate-hydroxymethylglutarate CoA-transferase isoform 2) c4(73504) c5(73505, 73506, 73507, 73508, 73509, 73510) c6( ) c7(aC, j, k, Ea, uu, cJH, fq, cd, ad); #c1(SUGCT) c2(NP_001180242) c3(succinate-hydroxymethylglutarate CoA-transferase isoform 3) c4(73511) c5(73512, 73513, 73514, 73515, 73516, 73517) c6( ) c7(aC, j, k, Ea, uu, cJH, fq, cd, ad); #c1(SUGCT) c2(XP_011513829) c3(succinate-hydroxymethylglutarate CoA-transferase isoform X4) c4(306271) c5( ) c6( ) c7(aC, j, k, Ea, uu, cJH, fq, cd, ad); #c1(SUGCT) c2(XP_011513832) c3(succinate-hydroxymethylglutarate CoA-transferase isoform X7) c4( ) c5(306272, 306273, 306274, 306275, 306276, 306277) c6( ) c7(aC, j, k, Ea, uu, cJH, fq, cd, ad); #c1(SUGP1) c2(NP_757386) c3(SURP and G-patch domain-containing protein 1) c4(249108, 249109, 249110) c5(249111, 249112, 249113, 249114, 249115, 249116, 249117, 249118, 249119, 249120, 249121, 249122, 249123, 249124, 249125, 249126, 249127, 249128) c6( ) c7(T, if, aA, Vd, qN, ad, WY, oW, aT, K, ae, d, je, aW, aq, aF, BU, iM, c, dB, U, B); #c1(SUGT1) c2(NP_001124384) c3(suppressor of G2 allele of SKP1 homolog isoform SGT1B) c4(48754) c5(48755, 48756, 48757, 48758, 48759, 48760, 48761, 48762, 48763, 48764, 48765, 48766, 48767, 48768) c6( ) c7(cd); #c1(SUGT1) c2(NP_006695) c3(suppressor of G2 allele of SKP1 homolog isoform SGT1A) c4(167203, 167204) c5(167205, 167206, 167207, 167208, 167209, 167210, 167211, (67212) c6( ) c7(cd); #c1(SUGT1) c2(XP_011533194) c3(suppressor of G2 allele of SKP1 homolog isoform X2) c4(329329) c5(329330, 329331, 329332, 329333, 329334, 329335) c6( ) c7(cd); #c1(SULF1) c2(NP_055985) c3(extracellular sulfatase Sulf-1 precursor) c4(184891, 184892) c5(184893, 184894, 184895, 184896, 184897, 184898, 184899, 184900, 184901, 184902, 184903, 184904, 184905, 184906, 184907, 184908, 184909, 184910) c6( ) c7(ar, T, aC, iu, aE, AO, cF, aW, fG, aT, fZ, n, aA, aq, aF, m, bu, t, iK, bz, B); #c1(SULF1) c2(XP_011515796) c3(extracellular sulfatase Sulf-1 isoform X1) c4(308576, 308577, 308578, 308579) c5(308580, 308581, 308582, 308583, 308584, 308585, 308586, 308587, 308588, 308589) c6( ) c7(ar, T, aC, iu, aE, AO, cF, aW, fG, aT, fZ, n, aA, aq, aF, m, bu, t, ik, bz, B); #c1(SULF1) c2(XP_011515797) c3(extracellular sulfatase Sulf-1 isoform X3) c4( ) c5(308590, 308591, 308592, 308593, 308594, 308595, 308596, 308597, 308598, 308599, 308600, 308601, 308602, 308603, 308604, 308605, 308606, 308607, 308608, 308609) c6( ) c7(ar, T, aC, iu, aE, AO, cF, aW, fG, aT, fZ, n, aA, aq, aF, m, bu, t, ik, bz, B); #c1(SULF2) c2(NP_061325) c3(extracellular sulfatase Sulf-2 isoform a precursor) c4(200010, 200011) c5(200012, 200013, 200014, 200015, 200016, 200017, 200018, 200019, 200020, 200021, 200022, 200023, 200024, 200025, 200026, 200027) c6( ) c7(bL, T, wL, fG, yk, aw, fZ, aA, cJl, fd, v, cd, q, eA, eJ, dP, iu, aT, qt, aW, fe, aF, bu, mo, cm, bz, iP, LB, es, ar, eM, iA, B); #c1(SULF2) c2(NP_940998) c3(extracellular sulfatase Sulf-2 isoform b precursor) c4(260295) c5(260296, 260297, 260298, 260299, 260300, 260301) c6( ) c7(bL, T, wL, fG, yk, aw, fZ, aA, cJl, fd, v, cd, q, eA, eJ, dP, iu, aT, qt, aW, fe, af, bu, mo, cm, bz, iP, LB, es, ar, eM, iA, B); #c1(SULF2) c2(XP_011527218) c3(extracellular sulfatase Sulf-2 isoform X6) c4(321763) c5(321764, 321765, 321766, 321767, 321768, 321769) c6( ) c7(bL, T, wL, fG, yk, aw, fZ, aA, cJl, fd, v, cd, q, eA, eJ, dP, iu, aT, qt, aW, fe, aF, bu, mo, cm, bz, iP, LB, es, ar, eM, iA, B); #c1(SULT1A1) c2(NP_803878) c3(sulfotransferase 1A1 isoform a) c4(252670) c5(252671, 252672, 252673, 252674, 252675, 252676, 252677, 252678, 252679, 252680, 252681, 252682, 252683, 252684, 252685, 252686, 252687, 252688, 252689) c6( ) c7(bL, Gl, atT, cJJ, T, bM, aC, id, nZ, QO, if, ht, aA, bB, n, hy, gf, j, ci, qN, fw, q, ad, eA, av, aT, Gw, dl, hu, ae, ne, Yi, eL, zQ, avV, d, B, s, aW, ff, mQ, aeU, r, aq, aF, en, cl, gW, t, iK, bz, Ld, qa, iT, dB, bE, X, pP, mg, hL, Gu, oQ, lf, aed, QS, BA); #c1(SULT1A1) c2(NP_803880) c3(sulfotransferase (A1 isoform b) c4(252690, 252691) c5(252692, 252693, 252694, 252695, 252696, 252697, 252698, 252699, 252700, 252701, 252702) c6( ) c7(bL, Gl, atT, cJJ, T, bM, aC, id, nZ, QO, if, ht, aA, bB, n, hy, gf, j, ci, qN, fw, q, ad, eA, av, aT, Gw, dL, hu, ae, ne, Yi, eL, zQ, avV, d, B, s, aW, ff, mQ, aeU, r, aq, aF, en, cl, gW, t, iK, bz, Ld, qa, iT, dB, bE, X, pP, mg, hL, Gu, oQ, lf, aed, QS, BA); #c1(SULT1A2) c2(NP_803564) c3(sulfotransferase 1A2) c4( ) c5(252664, 252665, 252666, 252667, 252668, 252669) c6( ) c7(T, aW, r, aT, dL, j, ad); #c1(SULT1A2) c2(XP_006721139) c3(sulfotransferase 1A2 isoform X1) c4(293875, 293876) c5(293877, 293878, 293879, 293880, 293881, 293882, 293883, 293884, 293885, 293886) c6( ) c7(T, aW, r, aT, dL, j, ad); #c1(SULT1A4) c2(NP_001017390) c3(sulfotransferase 1A4) c4(26373) c5(26374, 26375, 26376, 26377, 26378, 26379, 26380, 26381, 26382, 26383, 26384, 26385, 26386, 26387, 26388, 26389, 26390, 26391, 26392, 26393, 26394, 26395, 26396, 26397, 26398, 26399) c6( ) c7(eF, Kd, eH); #c1(SULT1B1) c2(NP_055280) c3(sulfotransferase family cytosolic 18 member 1) c4(180253, 180254) c5(180255, 180256, 180257, 180258, 180259, 180260, 180261, 180262, 180263, 180264, 180265, 180266) c6( ) c7(aw); #c1(SULT1C2) c2(NP_001047) c3(sulfotransferase 1C2 isoform a) c4(34349, 34350) c5(34351, 34352, 34353, 34354, 34355, 34356, 34357, 34358, 34359, 34360, 34361, 34362) c6( ) c7(BN, aW, aT, hy, bt, ne); #c1(SULT1C2) c2(NP_789795) c3(sulfotransferase 1C2 isoform b) c4(252342) c5(252343, 252344, 252345, 252346, 252347, 252348) c6( ) c7(BN, aW, aT, hy, bt, ne); #c1(SULT1E1) c2(NP_005411) c3(estrogen sulfotransferase) c4(155834) c5(155835, 155836, 155837, 155838, 155839, 155840) c6( ) c7(DS, T, cJJ, RX, al, aA, bB, n, bZ, v, Fb, ci, nA, Fc, pd, eA, ad, kL, aE, M, aT, iv, lG, rO, ae, al, kl, lR, TY, dH, Jl, aW, r, fQ, aq, aF, bu, cl, t, iK, bz, Ld, gk, c, dB, bE, hL, cj, hR, m, bgi, Bh, B, ao); #c1(SULT2A1) c2(NP_003158) c3(bile salt sulfotransferase) c4(135773) c5(135774, 135775, 135776, 135777, 135778, 135779, 135780) c6( ) c7(bM, T, lT, aZ, if, aA, fd, ci, qN, eA, ad, aT, h, By, dL, bK, uV, Xb, ae, bc, yo, akV, avH, kl, s, rh, aHW, aW, Y, hl, fQ, as, cl, eE, iV, PF, adz, bN, aBs, AV, HW, bQN, y, B); #c1(SULT2B1) c2(NP_004596) c3(sulfotransferase family cytosolic 28 member 1 isoform a) c4(148013) c5(148014, 148015, 148016, 148017, 148018, 148019) c6( ) c7(aW, aw, jx, r, aT, q, bX, cl, ae, B, ad); #c1(SULT2B1) c2(NP_814444) c3(sulfotransferase family cytosolic 28 member 1 isoform b) c4(253033) c5(253034, 253035, 253036, 253037, 253038, 253039) c6( ) c7(aW, aw, jx, r, aT, q, bX, cl, ae, B, ad); #c1(SULT4A1) c2(NP_055166) c3(sulfotransferase 4A1) c4(179549, (79550) c5(179551, 179552, 179553, 179554, 179555, 179556, 179557, 179558, 179559, 179560, 179561, 179562) c6( ) c7(yo, iH, BN, Y, HB, hy, ci, bX, ne); #c1(SULT4A1) c2(XP_011528423) c3(sulfotransferase 4A1 isoform X2) c4(323531) c5(323532, 323533, 323534, 323535, 323536, 323537) c6( ) c7(yo, iH, BN, Y, HB, hy, ci, bX, ne); #c1(SUMF1) c2(NP_001158146) c3(sulfatase-modifying factor 1 isoform 2 precursor) c4(63189) c5(63190, 63191, 63192, 63193, 63194, 63195) c6( ) c7(IE, kB, oe, agV, ade, V); #c1(SUMF1) c2(NP_001158147) c3(sulfatase-modifying factor 1 isoform 3 precursor) c4(63196) c5(63197, 63198, 63199, 63200, 63201, 63202) c6( ) c7(IE, kB, oe, agV, ade, V); #c1 (SUMF1) c2(NP_877437) c3(sulfatase-modifying factor 1 isoform 1 precursor) c4(256813) c5(256814, 256815, 256816, 256817, 256818, 256819) c6( ) c7(IE, kB, oe, agV, ade, V); #c1(SUMF1) c2(XP_011531926) c3(sulfatase-modifying factor 1 isoform X2) c4(327961, 327962) c5(327963, 327964, 327965, 327966, 327967, 327968, 327969, 327970, 327971, 327972, 327973, 327974) c6( ) c7(IE, KB, oe, agV, ade, V); #c1(SUMF2) c2(NP_001035934) c3(sulfatase-modifying factor 2 isoform c precursor) c4(33471) c5(33472, 33473, 33474, 33475, 33476, 33477) c6( ) c7(azU, RG, dc, qr, tv, tu, aaW, BF, bC, auu, pJ, aFf, ao); #c1(SUMF2) c2(NP_001035935) c3(sulfatase-modifying factor 2 isoform d precursor) c4(33478) c5(33479, 33480, 33481, 33482, 33483, 33484) c6( ) c7(azU, RG, dc, qr, tv, tu, aaW, BF, bC, auu, pJ, aFf, ao); #c1(SUMF2) c2(NP_001123541) c3 (sulfatase-modifying factor 2 isoform e precursor) c4(47460) c5(47461, 47462, 47463, 47464, 47465, 47466) c6( ) c7(azU, RG, dc, qr, tv, tu, aaW, BF, bC, auu, pJ, aFf, ao); #c1(SUMF2) c2(NP_001139805) c3(sulfatase-modifying factor 2 isoform g) c4( ) c5(59985, 59986, 59987, 59988, 59989) c6( ) c7(azU, RG, dc, qr, tv, tu, aaW, BF, bC, auu, pJ, aFf, ao); #c1(SUMF2) c2(NP_056226) c3(sulfatase-modifying factor 2 isoform b precursor) c4(186590, 186591) c5(186592, 186593, 186594, 186595, 186596, 186597, 186598) c6( ) c7(azU, RG, dc, qr, tv, tu, aaW, BF, bC, auu, pJ, aFf, ao); #c1(SUMF2) c2(XP_011513556) c3(sulfatase-modifying factor 2 isoform X1) c4(305843) c5(305844, 305845, 305846, 305847, 305848, 305849) c6( ) c7(azU, RG, dc, qr, tv, tu, aaW, BF, bC, auu, pJ, aFf, ao); #c1(SUMO1) c2(NP_001005781) c3(small ubiquitin-related modifier 1 isoform a precursor) c4(22049, 22050) c5(22051, 22052, 22053, 22054, 22055, 22056, 22057, 22058, 22059, 22060, 22061, 22062) c6( ) c7(T, acl, cd, ad, eA, Na, Db, yt, cJK, amJ, QJ, aT, dz, dL, Nc, jj, aVg, ae, eL, pi, aOX, A, d, aW, ff, rU, aq, en, hJ, QK, eF, bz, cm, ck, apM, bb, rR, c, eM, dB, U, bD, cst, bt, kS, B, y); #c1(SUMO1) c2(NP_001005782) c3(small ubiquitin-related modifier 1 isoform b precursor) c4(22063) c5(22064, 22065, 22066, 22067, 22068, 22069, 22070, 22071, 22072, 22073, 22074, 22075, 22076, 22077) c6( ) c7(T, acl, cd, ad, eA, Na, Db, yt, cJK, amJ, QJ, aT, dz, dL, Nc, jj, aVg, ae, eL, pi, aOX, A, d, aW, ff, rU, aq, en, hJ, QK, eF, bz, cm, ck, apM, bb, rR, c, eM, dB, U, bD, cst, bt, kS, B, y); #c1(SUMO2) c2(NP_001005849) c3(small ubiquitin-related modifier 2 isoform b precursor) c4(22116) c5(22117, 22118, 22119, 22120, 22121, 22122) c6( ) c7(ae, rR, aXK, aW, rU, ew, aT, iY, K, YH, jj, y, kS, pi, ad); #c1(SUMO2) c2(NP_008868) c3(small ubiquitin-related modifier 2 isoform a precursor) c4(169325, 169326) c5(169327, 169328, 169329, 169330, 169331, 169332, 169333, 169334, 169335, 169336, 169337, 169338) c6( ) c7(ae, rR, aXK, aW, rU, ew, aT, iY, K, YH, jj, y, kS, pi, ad); #c1(SUMO3) c2(NP_001273345) c3(small ubiquitin-related modifier 3 isoform 2) c4(104712) c5(104713, 104714, 104715, 104716, 104717, 104718) c6( ) c7(ae, rR, aXK, aW, ew, aT, iY, K, YH, y, kS, pi, ad); #c1(SUMO3) c2(NP_008867) c3(small ubiquitin-related modifier 3 isoform 1 precursor) c4(169318) c5(169319, 169320, 169321, 169322, 169323, 169324) c6( ) c7(ae, rR, aXK, aW, ew, aT, iY, K, YH, y, kS, pi, ad); #c1(SUMO4) c2(NP_001002255) c3(small ubiquitin-related modifier 4) c4(19448) c5(19449, 19450, 19451, 19452, 19453, 19454) c6( ) c7(bL, yG, cl, aj, j, v, uE, ml, o, J, uT, cJL, HD, hH, yF, A, HA, aGQ, cr, eZ, vy, fL, R, y, B); #c1(SUN1) c2(NP_001124437) c3(SUN domain-containing protein 1 isoform a) c4(48865, 48866, 48867, 48868, 48869) c5(48870, 48871, 48872, 48873, 48874, 48875, 48876, 48877, 48878, 48879, 48880, 48881, 48882, 48883, 48884, 48885, 48886, 48887, 48888, 48889, 48890, 48891, 48892, 48893, 48894, 48895) c6( ) c7(ac, MR, xs, rC, uY, Fw); #c1(SUN1) c2(NP_001165415) c3(SUN domain-containing protein 1 isoform c) c4(67667, 67668, 67669, 67670, 67671) c5(67672, 67673, 67674, 67675, 67676, 67677) c6( ) c7(ac, MR, xs, rC, uY, Fw); #c1(SUN1) c2(NP_001165416) c3(SUN domain-containing protein 1 isoform d) c4(67678) c5(67679, 67680, 67681, 67682, 67683, 67684, 67685, 67686, 67687, 67688, 67689, 67690, 67691) c6( ) c7(ac, MR, xs, rC, uY, Fw); #c1(SUN1) c2(NP_001165417) c3(SUN domain-containing protein 1 isoform e) c4(67692) c5(67693, 67694, 67695, 67696, 67697, 67698, 67699, 67700, 67701, 67702, 67703, 67704, 67705, 67706, 67707, 67708, 67709, 67710, 67711, 67712, 67713, 67714, 67715, 67716, 67717, 67718, 67719, 67720, 67721, 67722, 67723, 67724, 67725) c6( ) c7(ac, MR, xs, rC, uY, Fw); #c1(SUN1) c2(NP_079430) c3(SUN domain-containing protein 1 isoform b) c4(218093, 218094) c5(218095, 218096, 218097, 218098, 218099, 218100, 218101, 218102, 218103, 218104, 218105, 218106, 218107, 218108) c6( ) c7(ac, MR, xs, rC, uY, Fw); #c1(SUN2) c2(NP_001186508) c3(SUN domain-containing protein 2 isoform a) c4(77728, 77729) c5(77730, 77731, 77732, 77733, 77734, 77735) c6( ) c7(anR, aCg, iW, rC, uY, Fw, lQ, bU, bz); #c1(SUN2) c2(NP_001186509) c3(SUN domain-containing protein 2 isoform b) c4(77736, 77737, 77738, 77739) c5(77740, 77741, 77742, 77743, 77744, 77745, 77746, 77747, 77748, 77749, 77750, 77751, 77752, 77753, 77754, 77755, 77756, 77757, 77758, 77759, 77760, 77761, 77762, 77763, 77764, 77765, 77766, 77767, 77768, 77769, 77770, 77771, 77772, 77773, 77774, 77775) c6( ) c7(anR, aCg, iW, rC, uY, Fw, lQ, bU, bz); #c1(SUN2) c2(XP_011528406) c3(SUN domain-containing protein 2 isoform X1) c4(323501, 323502, 323503) c5(323504, 323505, 323506, 323507, 323508, 323509, 323510, 323511, 323512, 323513, 323514, 323515, 323516, 323517, 323518) c6( ) c7(anR, aCg, iW, rC, uY, Fw, lQ, bU, bz); #c1(SUN2) c2(XP_011528407) c3(SUN domain-containing protein 2 isoform X2) c4(323519) c5(323520, 323521, 323522, 323523, 323524, 323525, 323526, 323527, 323528, 323529, 323530) c6( ) c7(anR, aCg, iW, rC, uY, Fw, lQ, bU, bz); #c1(SUN3) c2(NP_001025190) c3(SUN domain-containing protein 3 isoform 1) c4(29491, 29492) c5(29493, 29494, 29495, 29496, 29497, 29498, 29499, 29500, 29501, 29502) c6( ) c7(c, an); #c1(SUN3) c2(NP_001271279) c3(SUN domain-containing protein 3 isoform 2) c4(103820) c5(103821, 103822, 103823, 103824, 103825, 103826, 103827, 103828, 103829, 103830, 103831, 103832, 103833) c6( ) c7(c, an); #c1(SUN3) c2(XP_011513554) c3(SUN domain-containing protein 3 isoform X1) c4(305832) c5(305833) c6( ) c7(c, an); #c1(SUN3) c2(XP_011513555) c3(SUN domain-containing protein 3 isoform X2) c4( ) c5(305834, 305835, 305836, 305837, 305838, 305839, 305840, 305841, 305842) c6( ) c7(c, an); #c1(SUOX) c2(NP_000447) c3(sulfite oxidase, mitochondrial) c4(9092, 9093) c5(9094, 9095, 9096, 9097, 9098, 9099, 9100, 9101, 9102, 9103, 9104, 9105, 9106, 9107, 9108) c6( ) c7(bM, mO, A, OW, fc, cod); #c1(SUOX) c2(XP_005269169) c3(sulfite oxidase, mitochondrial isoform X1) c4(282389, 282390) c5(282391, 282392, 282393, 282394, 282395, 282396, 282397, 282398, 282399, 282400, 282401, 282402, 282403, 282404, 282405, 282406, 282407, 282408, 282409, 282410, 282411, 282412, 282413, 282414, 282415, 282416, 282417, 282418, 282419) c6( ) c7(bM, mO, A, OW, fc, cod); #c1(SUPT2OH) c2(NP_001014308) c3 (transcription factor SPT20 homolog isoform a) c4(25804) c5(25805, 25806, 25807, 25808, 25809, 25810, 25811, 25812, 25813, 25814, 25815, 25816) c6( ) c7(es, T, iu, py, aW, if, aT, aF, ci, qN, ae, B, ad); #c1(SUPT2OH) c2(NP_060039) c3(transcription factor SPT20 homolog isoform b) c4(193831) c5(193832, 193833, 193834, 193835, 193836, 193837, 193838, 193839, 193840, 193841, 193842, 193843, 193844, 193845, 193846) c6( ) c7(es, T, iu, py, aW, if, aT, aF, ci, qN, a, B, ad); #c1(SUPT2OH) c2(XP_005266515) c3(transcription factor SPT20 homolog isoform X5) c4(280211) c5( ) c6( ) c7(es, T, iu, py, aW, if, aT, af, ci, qN, ae, B, ad); #c1(SUPT2OH) c2(XP_005266522) c3(transcription factor SPT20 homolog isoform X12) c4(280212) c5( ) c6( ) c7(es, T, iu, py, aW, if, aT, aF, ci, qN, ae, B, ad); #c1(SUPT2OH) c2(XP_005266525) c3(transcription factor SPT20 homolog isoform X14) c4( ) c5(280213, 280214, 280215, 280216, 280217, 280218) c6( ) c7(es, T, iu, py, aW, if, aT, aF, ci, qN, ae, B, ad); #c1(SUPT3H) c2(NP_001248752) c3(transcription initiation protein SPT3 homolog isoform 3) c4( ) c5(93989, 93990, 93991, 93992, 93993, 93994) c6( ) c7(Kw, U); #c1(SUPT3H) c2(NP_003590) c3(transcription initiation protein SPT3 homolog isoform 1) c4(139401, 139402) c5(139403, 139404, 139405, 139406, 139407, 139408, 139409, 139410, 139411, 139412, 139413, 139414, 139415, 139416, 139417) c6( ) c7(Kw, U); #c1(SUPT3H) c2(NP_852001) c3 (transcription initiation protein SPT3 homolog isoform 2) c4(254788) c5(254789, 254790, 254791, 254792, 254793, 254794, 254795, 254796, 254797, 254798) c6( ) c7(Kw, U); #c1(SUPT4H1) c2(NP_003159) c3(transcription elongation factor SPT4) c4(135781) c5(135782, 135783, 135784, 135785, 135786, 135787, 135788) c6( ) c7(on); #c1(SUPT7L) c2(NP_001269658) c3(STAGA complex 65 subunit gamma isoform a) c4(102425) c5(102426, 102427, 102428, 102429, 102430, 102431) c6( ) c7(T, dA, bfY, qb, ew, aA, jh, aj, lR, fu, fr, hF, V, akW, eA, J, wD, aMp, x, Bl, bOc, fh, oG, oW, aT, kH, CU, aLr, hn, h, lQ, bDk, afU, DY, ao, Ft, A, cJM, je, iU, xR, aW, Y, cr, eZ, gl, lo, aF, pD, hp, afq, hf, aot, iV, bw, bz, pb, jg, aHi, ck, es, ou, BU, pa, Fr, iM, avS, y, hk, eU, cj, wR, bfX, m, k, pw, bC, gT, bt, ei, B, hv); #c1(SUPT7L) c2(NP_001269660) c3(STAGA complex 65 subunit gamma isoform b) c4(102432) c5(102433, 102434, 102435, 102436, 102437, 102438, 102439, 102440, 102441, 102442, 102443, 102444, 102445, 102446) c6( ) c7(T, dA, bfY, qb, ew, aA, jh, aj, lR, fu, fr, hF, V, akW, eA, J, wD, aMp, x, Bl, bOc, fh, oG, oW, aT, kH, CU, aLr, hn, h, lQ, bDk, afU, DY, ao, Ft, A, cJM, je, iU, xR, aW, Y, cr, eZ, gl, lo, aF, pD, hp, afq, hf, aot, iV, bw, bz, pb, jg, aHi, ck, es, ou, BU, pa, Fr, iM, avS, y, hk, eU, cj, wR, bfX, m, k, pw, bC, gT, bt, ei, B, hv); #c1(SUPT7L) c2(NP_001269661) c3(STAGA complex 65 subunit gamma isoform c) c4(102447) c5(102448, 102449, 102450, 102451, 102452, 102453) c6( ) c7(T, dA, bfY, qb, ew, aA, jh, aj, lR, fu, fr, hF, V, akW, eA, J, wD, aMp, x, Bl, bOc, fh, OG, oW, aT, KH, CU, aLr, hn, h, lQ, bDk, afU, DY, ao, Ft, A, cJM, je, iU, xR, aW, Y, cr, eZ, gl, lo, aF, pD, hp, afq, hf, aot, iV, bw, bz, pb, jg, aHi, ck, es, ou, BU, pa, Fr, iM, avS, y, hk, eU, cj, wR, bfX, m, k, pw, bC, gT, bt, ei, B, hv); #c1(SUPV3L1) c2(NP_003162) c3(ATP-dependent RNA helicase SUPV3L1, mitochondrial isoform 1) c4(135789, 135790) c5(135791, 135792, 135793, 135794, 135795, 135796, 135797) c6( ) c7(li, T, sF, aT, aeM, af); #c1(SUPV3L1) c2(XP_011538409) c3(ATP-dependent RNA helicase SUPV3L1, mitochondrial isoform X2) c4( ) c5(336093, 336094, 336095, 336096, 336097, 336098) c6( ) c7(li, T, sF, aT, aeM, af); #c1(SURF1) c2(NP_003163) c3(surfeit locus protein 1 isoform 1) c4(135798, 135799) c5(135800, 135801, 135802, 135803, 135804, 135805, 135806, 135807, 135808, 135809, 135810, 135811) c6( ) c7(ob, agU, aAs, caG, nA, bX, aR, vc, aln, bU, cb, ajU, Az, aUx, mv, kC, ky, SE, U, oh, acJ, it); #c1(SURF4) c2(NP_001267718) c3(surfeit locus protein 4 isoform 3) c4( ) c5(99653, 99654, 99655, 99656, 99657, 99658) c6( ) c7(ad); #c1(SURF4) c2(NP_001267721) c3(surfeit locus protein 4 isoform 5) c4(99659) c5(99660, 99661, 99662, 99663, 99664) c6( ) c7(ad); #c1(SURF4) c2(NP_149351) c3(surfeit locus protein 4 isoform 1) c4(227460, 227461, 227462) c5(227463, 227464, 227465, 227466, 227467, 227468, 227469, 227470, 227471, 227472, 227473, 227474) c6( ) c7(ad); #c1(SURF4) c2(XP_011517246) c3(surfeit locus protein 4 isoform X1) c4(310829) c5(310830, 310831, 310832, 310833, 310834, 310835) c6( ) c7(ad); #c1(SURF4) c2(XP_011517247) c3(surfeit locus protein 4 isoform X2) c4( ) c5(310836) c6( ) c7(ad); #c1(SUSD1) c2(NP_001269569) c3(sushi domain-containing protein 1 isoform 1 precursor) c4(101999) c5(102000, 102001, 102002, 102003, 102004, 102005) c6( ) c7(Na, Nc, c); #c1(SUSD1) c2(NP_001269572) c3(sushi domain-containing protein 1 isoform 3 precursor) c4(102006) c5(102007, 102008, 102009, 102010, 102011, 102012) c6( ) c7(Na, Nc, c); #c1(SUSD1) c2(NP_071931) c3(sushi domain-containing protein 1 isoform 2 precursor) c4(212380) c5(212381, 212382, 212383, 212384, 212385, 212386) c6( ) c7(Na, Nc, c); #c1(SUSD2) c2(NP_062547) c3(sushi domain-containing protein 2 precursor) c4(201936) c5(201937, 201938, 201939, 201940, 201941, 201942) c6( ) c7(aW, c, aT); #c1(SUSD4) c2(NP_001032252) c3(sushi domain-containing protein 4 isoform b precursor) c4(31184) c5(31185, 31186, 31187, 31188, 31189, 31190, 31191, 31192, 31193, 31194, 31195, 31196, 31197, 31198, 31199, 31200, 31201, 31202, 31203, 31204, 31205, 31206) c6( ) c7(aw, bX); #c1(SUSD4) c2(NP_060452) c3(sushi domain-containing protein 4 isoform a precursor) c4(196001, 196002, 196003, 196004) c5(196005, 196006, 196007, 196008, 196009, 196010, 196011, 196012, 196013, 196014, 196015) c6( ) c7(aw, bX); #c1(SUSD4) c2(XP_005273229) c3(sushi domain-containing protein 4 isoform X4) c4( ) c5(284380, 284381, 284382, 284383, 284384) c6( ) c7(aw, bX); #c1(SUSD4) c2(XP_011507988) c3(sushi domain-containing protein 4 isoform X2) c4(298429) c5(298430, 298431, 298432, 298433, 298434, 298435) c6( ) c7(aw, bX); #c1(SUSD6) c2(NP_055549) c3(uncharacterized protein KIAA0247 precursor) c4(181889) c5(181890, 181891, 181892, 181893, 181894, 181895) c6( ) c7(q, r); #c1(SUV39H1) c2(NP_001269095) c3(histone-lysine N-methyltransferase SUV39H1 isoform 1) c4(100431) c5(100432, 100433, 100434, 100435, 100436, 100437) c6( ) c7(ck, T, A, Qr, Bc, mC, aW, eZ, aT, r, n, aj, dG, K, fl, q, Xu, bt, B, J, ad); #c1(SUV39H1) c2(NP_003164) c3(histone-lysine N-methyltransferase SUV39H1 isoform 2) c4(135812, 135813, 135814) c5(135815, 135816, 135817, 135818, 135819, 135820, 135821, 135822, 135823, 135824, 135825, 135826, 135827, 135828, 135829, 135830, 135831, 135832, 135833, 135834, 135835, 135836, 135837, 135838) c6( ) c7(ck, T, A, Qr, Bc, mC, aW, eZ, aT, r, n, aj, dG, K, fl, q, Xu, bt, B, J, ad); #c1(SUV39H2) c2(NP_001180353) c3(histone-lysine N-methyltransferase SUV39H2 isoform 1) c4(73865, 73866) c5(73867, 73868, 73869, 73870, 73871, 73872) c6( ) c7(av, bz, pb, A, vy); #c1(SUV39H2) c2(NP_001180354) c3(histone-lysine N-methyltransferase SUV39H2 isoform 2) c4( ) c5(73873, 73874, 73875, 73876, 73877) c6( ) c7(av, bz, pb, A, vy); #c1(SUV39H2) c2(NP_001180355) c3(histone-lysine N-methyltransferase SUV39H2 isoform 3) c4(73878, 73879) c5(73880, 73881, 73882, 73883, 73884, 73885, 73886) c6( ) c7(av, bz, pb, A, vy); #c1(SUV39H2) c2(NP_001180356) c3(histone-lysine N-methyltransferase SUV39H2 isoform 4) c4( ) c5(73887, 73888, 73889, 73890, 73891) c6( ) c7(av, bz, pb, A, vy); #c1(SUV39H2) c2(XP_011517963) c3(histone-lysine N-methyltransferase SUV39H2 isoform X2) c4( ) c5(312008) c6( ) c7(av, bz, pb, A, vy); #c1(SUV420H2) c2(NP_116090) c3(histone-lysine N-methyltransferase SUV420H2) c4(225339) c5(225340, 225341, 225342, 225343, 225344, 225345, 225346) c6( ) c7(aq, aT, bq); #c1(SUZ12) c2(NP_056170) c3(polycomb protein SUZ12) c4(186095) c5(186096, 186097, 186098, 186099, 186100, 186101) c6( ) c7(T, if, aA, ci, qN, q, br, ad, hX, eA, aeO, aT, dL, hP, ae, eL, dx, d, aeR, iU, aW, Y, r, aq, aF, aiz, cl, iR, aEm, bw, Ew, dB, lp, hL, m, Th, bt, KS, B); #c1(SV2B) c2(NP_055663) c3(synaptic vesicle glycoprotein 28 isoform 1) c4(182598, 182599) c5(182600, 182601, 182602, 182603, 182604, 182605, 182606, 182607, 182608, 182609, 182610, 182611, 182612, 182613, 182614, 182615, 182616, 182617, 182618, 182619) c6( ) c7(Z, hF, y, dy, ac); #c1(SV2C) c2(NP_001284645) c3(synaptic vesicle glycoprotein 2C isoform 2) c4(112236) c5(112237, 112238, 112239, 112240, 112241, 112242) c6( ) c7(VZ, cz); #c1(SV2C) c2(NP_055794) c3(synaptic vesicle glycoprotein 2C isoform 1) c4(183549) c5(183550, 183551, 183552, 183553, 183554, 183555) c6( ) c7(VZ, cz); #c1(SVEP1) c2(NP_699197) c3(sushi, von Willebrand factor type A, EGF and pentraxin domain-containing protein 1 precursor) c4(245849, 245850, 245851, 245852, 245853, 245854) c5(245855, 245856, 245857, 245858, 245859, 245860, 245861, 245862, 245863, 245864, 245865, 245866, 245867, 245868, 245869, 245870, 245871, 245872, 245873, 245874, 245875, 245876, 245877, 245878, 245879, 245880, 245881, 245882, 245883, 245884, 245885, 245886, 245887, 245888, 245889, 245890) c6( ) c7(aW, ahW, ahl, aT); #c1(SVIL) c2(NP_003165) c3(supervillin isoform 1) c4(135839) c5(135840, 135841, 135842, 135843, 135844, 135845) c6( ) c7(es, bt, aj, fr, K, aZf, V); #c1(SVIL) c2(NP_068506) c3(supervillin isoform 2) c4(209380, 209381) c5(209382, 209383, 209384, 209385, 209386, 209387, 209388, 209389, 209390, 209391, 209392) c6( ) c7(es, bt, aj, fr, K, aZf, V); #c1(SVIL) c2(XP_011517932) c3(supervillin isoform X1) c4(311936) c5(311937, 311938, 311939, 311940, 311941, 311942) c6( ) c7(es, bt, aj, fr, K, aZf, V); #c1(SVIL) c2(XP_011517935) c3(supervillin isoform X4) c4(311943) c5(311944, 311945, 311946, 311947, 311948) c6( ) c7(es, bt, aj, fr, K, aZf, V); #c1(SVIL) c2(XP_011517940) c3(supervillin isoform X8) c4( ) c5(311949, 311950, 311951, 311952, 311953, 311954) c6( ) c7(es, bt, aj, fr, K, aZf, V); #c1(SVIP) c2(NP_683691) c3(small VCP/p97-interacting protein) c4(242380) c5(242381, 242382, 242383, 242384, 242385) c6( ) c7(bL, cd); #c1(SVOP) c2(NP_061181) c3(synaptic vesicle 2-related protein) c4(199804) c5(199805, 199806, 199807, 199808, 199809, 199810, 199811, 199812, 199813, 199814, 199815) c6( ) c7(j, bX); #c1(SWAP70) c2(NP_055870) c3(switch-associated protein 70 isoform 1) c4(184040) c5(184041, 184042, 184043, 184044, 184045, 184046) c6( ) c7(t, ad, m, fR, ae); #c1(SWT1) c2(NP_060143) c3(transcriptional protein SWT1) c4(194427, 194428) c5(194429, 194430, 194431, 194432, 194433, 194434, 194435, 194436, 194437, 194438, 194439, 194440, 194441, 194442, 194443, 194444) c6( ) c7(ck, bt, jj); #c1(SYBU) c2(NP_001093213) c3(syntabulin isoform b) c4(39955, 39956, 39957, 39958) c5(39959, 39960, 39961, 39962, 39963, 39964, 39965, 39966, 39967, 39968, 39969, 39970, 39971, 39972, 39973, 39974, 39975, 39976, 39977, 39978, 39979, 39980, 39981, 39982, 39983, 39984, 39985, 39986, 39987, 39988, 39989, 39990, 39991, 39992, 39993, 39994, 39995, 39996, 39997, 39998, 39999, 40000, 40001, 40002, 40003) c6( ) c7(hl, GC, C, cd, ad, Hx, cbH, aE, h, cy, K, XZ, ae, af, d, aW, aml, Y, ff, en, U, m, B); #c1(SYBU) c2(NP_001093214) c3(syntabulin isoform a) c4(40004, 40005, 40006, 40007, 40008, 40009) c5(40010, 40011, 40012, 40013, 40014, 40015, 40016, 40017, 40018, 40019, 40020, 40021, 40022, 40023, 40024, 40025, 40026, 40027, 40028, 40029, 40030, 40031, 40032, 40033, 40034, 40035, 40036, 40037, 40038, 40039, 40040, 40041, 40042, 40043, 40044, 40045, 40046, 40047, 40048, 40049, 40050, 40051, 40052, 40053, 40054, 40055, 40056, 40057, 40058, 40059, 40060, 40061, 40062, 40063, 40064, 40065, 40066, 40067, 40068, 40069, 40070, 40071, 40072, 40073, 40074, 40075, 40076, 40077, 40078, 40079) c6( ) c7(hl, GC, C, cd, ad, Hx, cbH, aE, h, cy, K, XZ, ae, af, d, aW, aml, Y, ff, en, U, m, B); #c1(SYBU) c2(NP_001093225) c3(syntabulin isoform d) c4(40080, 40081, 40082, 40083, 40084) c5(40085, 40086, 40087, 40088, 40089, 40090, 40091, 40092, 40093, 40094, 40095, 40096, 40097, 40098, 40099, 40100, 40101, 40102, 40103, 40104, 40105, 40106, 40107, 40108) c6( ) c7(hl, GC, C, cd, ad, Hx, cbH, aE, h, cy, K, XZ, ae, af, d, aW, aml, Y, ff, en, U, m, B); #c1(SYBU) c2(NP_001093226) c3(syntabulin isoform c) c4(40109, 40110) c5(40111, 40112, 40113, 40114, 40115, 40116, 40117, 40118, 40119, 40120, 40121) c6( ) c7(hl, GC, C, cd, ad, Hx, cbH, aE, h, cy, K, XZ, ae, af, d, aW, aml, Y, ff, en, U, m, B); #c1(SYCE1) c2(NP_001137235) c3(synaptonemal complex central element protein 1 isoform 3) c4(55087, 55088) c5(55089, 55090, 55091, 55092, 55093, 55094, 55095, 55096) c6( ) c7(vJ, il); #c1(SYCE1) c2(NP_001137236) c3(synaptonemal complex central element protein 1 isoform 4) c4(55097) c5(55098, 55099, 55100, 55101, 55102, 55103) c6( ) c7(vJ, il); #c1(SYCE1) c2(NP_570140) c3(synaptonemal complex central element protein 1 isoform 1) c4( ) c5(234057, 234058, 234059, 234060, 234061, 234062, 234063, 234064) c6( ) c7(vJ, il); #c1(SYCE1L) c2(NP_001123451) c3(synaptonemal complex central element protein 1-like) c4(47093) c5(47094, 47095, 47096, 47097, 47098, 47099) c6( ) c7(T, ha, jx, fr, ci, lu, q, ft, F, ib, aE, jL, aT, bc, ls, aW, Y, kK, hC, ff, lo, aF, en, hf, hJ, g, bz, eM, m, hE, bt, B, hS); #c1(SYCP2) c2(NP_055073) c3(synaptonemal complex protein 2) c4(178775, 178776) c5(178777, 178778, 178779, 178780, 178781, 178782, 178783, 178784, 178785, 178786, 178787, 178788, 178789, 178790, 178791, 178792) c6( ) c7(vH, if, qN, af, ND); #c1(SYCP2L) c2(NP_001035364) c3(synaptonemal complex protein 2-like) c4(32823, 32824) c5(32825, 32826, 32827, 32828, 32829, 32830, 32831, 32832, 32833, 32834, 32835, 32836) c6( ) c7(gv); #c1(SYCP3) c2(NP_001171420) c3(synaptonemal complex protein 3) c4(70066) c5(70067, 70068, 70069, 70070, 70071, 70072, 70073, 70074, 70075, 70076, 70077, 70078, 70079) c6( ) c7(T, sn, iT, if, vH, aDC, qN, pt, bt, cJN); #c1(SYCP3) c2(XP_005268979) c3 (synaptonemal complex protein 3 isoform X1) c4( ) c5(282257) c6( ) c7(T, sn, iT, if, vH, aDC, qN, pt, bt, cJN); #c1(SYK) c2(XP_011517248) c3(tyrosine-protein kinase SYK isoform X1) c4(310837) c5(310838, 310839, 310840, 310841, 310842, 310843, 310844, 310845, 310846, 310847) c6( ) c7(bL, cxW, T, dD, fM, dA, rV, fR, ea, yk, iW, jh, cz, n, eq, ci, V, ai, x, azY, ft, ib, aT, Vv, dz, hn, cml, qt, aW, Y, ff, Of, eZ, Og, en, hf, mo, wA, bw, bz, ck, Ew, bP, cj, CZ, ME, k, fD, bt, B); #c1(SYK) c2(XP_011517249) c3(tyrosine-protein kinase SYK isoform X2) c4(310848) c5(310849, 310850, 310851, 310852, 310853, 310854, 310855, 310856, 310857, 310858) c6( ) c7(bL, cxW, T, dD, fM, dA, rV, fR, ea, yk, iW, jh, cz, n, eq, ci, V, ai, x, azY, ft, ib, aT, Vv, dz, hn, cml, qt, aW, Y, ff, Of, eZ, Og, en, hf, mo, wA, bw, bz, ck, Ew, bP, cj, CZ, ME, k, fD, bt, B); #c1(SYMPK) c2(XP_011525656) c3(symplekin isoform X1) c4(320067, 320068) c5(320069, 320070, 320071, 320072, 320073, 320074, 320075, 320076, 320077, 320078, 320079) c6( ) c7(aA); #c1(SYMPK) c2(XP_011525657) c3(symplekin isoform X2) c4( ) c5(320080) c6( ) c7(aA); #c1(SYN1) c2(NP_008881) c3(synapsin-1 isoform Ia) c4(169490) c5( ) c6( ) c7(bL, Qi, T, bb, aW, dR, ha, lr, aT, cm, avl, an, cJO, Fc, af, bN, L); #c1(SYN1) c2(NP_598006) c3(synapsin-1 isoform Ib) c4(235282) c5(235283, 235284, 235285, 235286, 235287, 235288, 235289, 235290, 235291, 235292, 235293, 235294, 235295, 235296, 235297, 235298) c6( ) c7(bL, Qi, T, bb, aW, dR, ha, lr, aT, cm, avl, an, cJO, Fc, af, bN, L); #c1(SYN2) c2(NP_003169) c3(synapsin-2 isoform IIb) c4( ) c5(135853) c6( ) c7(bL, nX, ha, avl, bK, af, bN, zn); #c1(SYN2) c2(NP_598328) c3(synapsin-2 isoform IIa) c4(235405) c5(235406, 235407, 235408, 235409, 235410, 235411) c6( ) c7(bL, nX, ha, avl, bK, af, bN, zn); #c1(SYN2) c2(XP_006713376) c3(synapsin-2 isoform X3) c4( ) c5(287164) c6( ) c7(bL, nX, ha, avl, bK, af, bN, zn); #c1(SYN3) c2(NP_598344) c3(synapsin-3 isoform IIIc) c4(235433) c5(235434, 235435, 235436, 235437, 235438, 235439, 235440, 235441, 235442, 235443, 235444, 235445, 235446, 235447, 235448, 235449) c6( ) c7(cF, W, wK, Vr, EQ, lr, n, aHU, dL, bK, Da, V, eL, bN); #c1(SYN3) c2(XP_011528711) c3(synapsin-3 isoform X1) c4(323964) c5( ) c6( ) c7(cF, W, wK, Vr, EQ, lr, n, aHU, dL, bK, Da, V, eL, bN); #c1(SYN3) c2(XP_011528712) c3(synapsin-3 isoform X2) c4(323965) c5( ) c6( ) c7(cF, W, wK, Vr, EQ, lr, n, aHU, dL, bK, Da, V, eL, bN); #c1(SYN3) c2(XP_011528714) c3(synapsin-3 isoform X4) c4( ) c5(323966, 323967, 323968, 323969, 323970, 323971) c6( ) c7(cF, W, wK, Vr, EQ, lr, n, aHU, dL, bK, Da, V, eL, bN); #c1(SYN3) c2(XP_011528715) c3(synapsin-3 isoform X5) c4(323972, 323973) c5( ) c6( ) c7(cF, W, wK, Vr, EQ, lr, n, aHU, dL, bk, Da, V, eL, bN); #c1(SYN3) c2(XP_011528716) c3(synapsin-3 isoform X6) c4( ) c5(323974, 323975, 323976, 323977, 323978, 323979, 323980, 323981, 323982, 323983, 323984, 323985) c6( ) c7(cF, W, wK, Vr, EQ, lr, n, aHU, dL, bK, Da, V, eL, bN); #c1(SYNCRIP) c2(NP_001153146) c3(heterogeneous nuclear ribonucleoprotein Q isoform 3) c4( ) c5(60452, 60453, 60454, 60455, 60456, 60457) c6( ) c7(kf, bB, bE, ey); #c1(SYNCRIP) c2(NP_001153148) c3(heterogeneous nuclear ribonucleoprotein [isoform 5) c4( ) c5(60458, 60459, 60460, 60461, 60462, 60463, 60464, 60465, 60466, 60467, 60468, 60469) c6( ) c7(kf, bB, bE, ey); #c1(SYNCRIP) c2(NP_001153149) c3(heterogeneous nuclear ribonucleoprotein Q isoform 6) c4(60470) c5( ) c6( ) c7(kf, bB, bE, ey); #c1(SYNCRIP) c2(NP_006363) c3(heterogeneous nuclear ribonucleoprotein Q isoform 1) c4(164379, 164380) c5( ) c6( ) c7(kf, bB, bE, ey); #c1(SYNDIG1) c2(NP_079169) c3(synapse differentiation-inducing gene protein 1) c4(217284) c5(217285, 217286, 217287, 217288, 217289, 217290) c6( ) c7(aC, gf, aw); #c1(SYNE1) c2(NP_149062) c3(nesprin-1 isoform 2) c4(226968, 226969, 226970) c5(226971, 226972, 226973, 226974, 226975, 226976, 226977, 226978, 226979, 226980, 226981, 226982) c6( ) c7(bL, T, oe, ht, ci, q, x, cc, cJP, aT, bK, K, ji, cJA, s, aW, Y, ff, xs, r, uY, en, g, t, qa, ck, Dy, ky, kB, gZ, Fw, m, k, lB, B); #c1(SYNE1) c2(NP_892006) c3(nesprin-1 isoform 1) c4(257450, 257451, 257452) c5(257453, 257454, 257455, 257456, 257457, 257458, 257459, 257460, 257461, 257462, 257463, 257464, 257465, 257466, 257467, 257468, 257469, 257470) c6( ) c7(bL, T, oe, ht, ci, q, x, cc, cJP, aT, bk, K, ji, cJA, s, aW, Y, ff, xs, r, uY, en, g, t, qa, ck, Dy, ky, kB, gZ, Fw, m, k, lB, B); #c1(SYNE1) c2(XP_006715475) c3(nesprin-1 isoform X8) c4( ) c5(289582, 289583, 289584, 289585, 289586, 289587) c6( ) c7(bL, T, oe, ht, ci, q, x, cc, cJP, aT, bK, K, ji, cJA, s, aW, Y, ff, xs, r, uY, en, g, t, qa, ck, Dy, ky, kB, gZ, Fw, m, k, lB, B); #c1(SYNE1) c2(XP_006715479) c3(nesprin-1 isoform X12) c4(289588) c5(289589, 289590, 289591, 289592, 289593, 289594, 289595, 289596, 289597, 289598, 289599, 289600) c6( ) c7(bL, T, oe, ht, ci, q, x, cc, cJP, aT, bk, K, ji, cJA, s, aW, Y, ff, xs, r, uY, en, g, t, qa, ck, Dy, ky, kB, gZ, Fw, m, k, lB, B); #c1(SYNE1) c2(XP_011533948) c3(nesprin-1 isoform X23) c4(330145) c5( ) c6( ) c7(bL, T, oe, ht, ci, q, x, cc, cJP, aT, bK, K, ji, cJA, s, aW, Y, ff, xs, r, uY, en, g, t, qa, ck, Dy, ky, kB, gZ, Fw, m, k, lB, B); #c1(SYNE2) c2(NP_055995) c3(nesprin-2 isoform 1) c4(185002, 185003, 185004, 185005) c5(185006, 185007, 185008, 185009, 185010, 185011, 185012, 185013, 185014, 185015, 185016, 185017, 185018, 185019, 185020, 185021, 185022, 185023, 185024, 185025, 185026, 185027, 185028, 185029, 185030, 185031, 185032, 185033) c6( ) c7(cJR, cc, wL, xs, Fw, fr); #c1(SYNE2) c2(NP_878914) c3(nesprin-2 isoform 2) c4(257167) c5(257168, 257169, 257170, 257171, 257172) c6( ) c7(cJR, cc, wL, xs, Fw, fr); #c1(SYNE2) c2(NP_878917) c3(nesprin-2 isoform 4) c4(257192) c5(257193, 257194, 257195, 257196, 257197, 257198) c6( ) c7(cJR, cc, wL, xs, Fw, fr); #c1(SYNE2) c2(NP_878918) c3 (nesprin-2 isoform 5) c4(257199, 257200) c5(257201, 257202, 257203, 257204, 257205, 257206, 257207, 257208, 257209, 257210, 257211, 257212) c6( ) c7(cJR, cc, wL, xs, Fw, fr); #c1(SYNE2) c2(XP_005267511) c3(nesprin-2 isoform X3) c4( ) c5(281016) c6( ) c7(cJR, cc, wL, xs, Fw, fr); #c1(SYNE2) c2(XP_006720147) c3(nesprin-2 isoform X13) c4(293294) c5(293295, 293296, 293297, 293298, 293299, 293300) c6( ) c7(cJR, cc, wL, xs, Fw, fr); #c1(SYNE2) c2(XP_011534879) c3(nesprin-2 isoform X1) c4( ) c5(331414, 331415, 331416, 331417, 331418, 331419) c6( ) c7(cJR, cc, wL, xs, Fw, fr); #c1(SYNE2) c2(XP_011534886) c3(nesprin-2 isoform X14) c4(331420) c5(331421) c6( ) c7(cJR, cc, wL, xs, Fw, fr); #c1(SYNE4) c2(NP_001034965) c3(nesprin-4 isoform 1) c4(32408) c5(32409, 32410, 32411, 32412, 32413, 32414) c6( ) c7(cJS, bCd); #c1(SYNE4) c2(NP_001284664) c3(nesprin-4 isoform 2) c4(112281) c5(112282, 112283, 112284, 112285, 112286, 112287) c6( ) c7(cJS, bCd); #c1(SYNGAP1) c2(NP_006763) c3(ras/Rap GTPase-activating protein SynGAP) c4(167821, 167822, 167823, 167824, 167825) c5(167826, 167827, 167828, 167829, 167830, 167831, 167832, 167833, 167834, 167835, 167836, 167837, 167838, 167839, 167840, 167841, 167842, 167843, 167844, 167845, 167846, 167847, 167848, 167849, 167850, 167851, 167852, 167853, 167854, 167855, 167856, 167857, 167858, 167859, 167860, 167861, 167862, 167863, 167864, 167865, 167866, 167867) c6( ) c7(bVl, cyw, Ck, bN, ot, kl, ha, ol, afy, eZ, cJT, nA, Cd, hg, rz, Cb, dr, xt); #c1(SYNGR1) c2(NP_004702) c3(synaptogyrin-1 isoform 1a) c4(148931, 148932) c5(148933, 148934, 148935, 148936, 148937, 148938) c6( ) c7(bt, bK); #c1(SYNGR1) c2(NP_663783) c3(synaptogyrin-1 isoform 1b) c4(241578, 241579) c5(241580, 241581, 241582, 241583, 241584, 241585, 241586, 241587, 241588, 241589, 241590, 241591, 241592, 241593, 241594, 241595, 241596, 241597) c6( ) c7(bt, bK); #c1(SYNGR1) c2(NP_663791) c3(synaptogyrin-1 isoform 1c) c4(241638) c5(241639, 241640, 241641, 241642, 241643, 241644) c6( ) c7(bt, bK); #c1(SYNGR2) c2(NP_004701) c3(synaptogyrin-2) c4(148924) c5(148925, 148926, 148927, 148928, 148929, 148930) c6( ) c7(T); #c1(SYNGR2) c2(XP_005257849) c3(synaptogyrin-2 isoform X1) c4( ) c5(275382, 275383) c6( ) c7(T); #c1(SYNJ1) c2(NP_001153774) c3(synaptojanin-1 isoform c) c4(60985) c5(60986, 60987, 60988, 60989, 60990) c6( ) c7(KC, bv, bAn, cJU, tx, cm, uJ, bk, f, nC, xg, af); #c1(SYNJ1) c2(NP_001153778) c3(synaptojanin-1 isoform d) c4(60991) c5(60992, 60993, 60994, 60995, 60996, 60997) c6( ) c7(KC, bv, bAn, cJU, tx, cm, uJ, bK, f, nC, xg, af); #c1(SYNJ1) c2(NP_003886) c3(synaptojanin-1 isoform a) c4(141806, 141807) c5(141808, 141809, 141810, 141811, 141812, 141813, 141814, 141815, 141816, 141817, 141818, 141819, 141820, 141821, 141822, 141823, 141824, 141825, 141826) c6( ) c7(KC, bv, bAn, cJU, tx, cm, uJ, bk, f, nC, xg, af); #c1(SYNJ1) c2(NP_982271) c3(synaptojanin-1 isoform b) c4(263248) c5(263249) c6( ) c7(KC, bv, bAn, cJU, tx, cm, uJ, bk, f, nC, xg, af); #c1(SYNJ2) c2(NP_001171559) c3(synaptojanin-2 isoform 2) c4( ) c5(70582, 70583, 70584, 70585, 70586, 70587) c6( ) c7(aT, gf, Jt, cd, ad, zn); #c1(SYNJ2) c2(NP_003889) c3(synaptojanin-2 isoform 1) c4(141895, 141896, 141897, 141898) c5(141899, 141900, 141901, 141902, 141903, 141904) c6( ) c7(aT, gf, Jt, cd, ad, zn); #c1(SYNJ2) c2(XP_005267257) c3(synaptojanin-2 isoform X7) c4( ) c5(280842) c6( ) c7(aT, gf, Jt, cd, ad, zn); #c1(SYNJ2) c2(XP_006715655) c3(synaptojanin-2 isoform X3) c4(289688) c5(289689, 289690, 289691, 289692, 289693, 289694) c6( ) c7(aT, gf, Jt, cd, ad, zn); #c1(SYNJ2) c2(XP_011534526) c3(synaptojanin-2 isoform X2) c4(331019) c5(331020, 331021, 331022, 331023, 331024, 331025) c6( ) c7(aT, gf, Jt, cd, ad, zn); #c1(SYNJ2) c2(XP_011534531) c3(synaptojanin-2 isoform X8) c4(331026) c5(331027, 331028, 331029, 331030, 331031, 331032, 331033, 331034, 331035, 331036, 331037, 331038) c6( ) c7(aT, gf, Jt, cd, ad, zn); #c1(SYNJ2) c2(XP_011534532) c3(synaptojanin-2 isoform X9) c4( ) c5(331039, 331040, 331041, 331042, 331043, 331044) c6( ) c7(aT, gf, Jt, cd, ad, zn); #c1(SYNJ2BP) c2(NP_060843) c3(synaptojanin-2-binding protein) c4(198092) c5(198093, 198094, 198095, 198096, 198097, 198098) c6( ) c7(aW, aT); #c1(SYNM) c2(NP_056101) c3(synemin isoform B) c4(185712, 185713) c5(185714, 185715, 185716, 185717, 185718, 185719, 185720, 185721, 185722, 185723, 185724, 185725) c6( ) c7(T, GC, n, Hr, Dq, oj, aT, K, an, li, ak, yo, Pw, d, s, aW, app, cm, hq, Bv, ky, U, aen, B); #c1(SYNM) c2(NP_663780) c3(synemin isoform A) c4(241555) c5(241556, 241557, 241558, 241559, 241560, 241561) c6( ) c7(T, GC, n, Hr, Dq, oj, aT, K, an, li, ak, yo, Pw, d, s, aW, app, cm, hq, Bv, ky, U, aen, B); #c1 (SYNPO) c2(NP_001103444) c3 (synaptopodin isoform B) c4(41907) c5( ) c6( ) c7(sk, dk, vw, ah, K, ey, uW, uC, ad); #c1(SYNPO) c2(NP_001159681) c3(synaptopodin isoform C) c4(64556) c5(64557, 64558, 64559, 64560, 64561, 64562, 64563, 64564, 64565, 64566, 64567, 64568, 64569, 64570, 64571, 64572) c6( ) c7(sk, dk, vw, ah, K, ey, uW, uC, ad); #c1(SYNPO) c2(NP_009217) c3(synaptopodin isoform A) c4(171564) c5(171565, 171566, 171567, 171568, 171569) c6( ) c7(sk, dk, vw, ah, K, ey, uW, uC, ad); #c1(SYNPO) c2(XP_006714818) c3(synaptopodin isoform X3) c4( ) c5(288248) c6( ) c7(sk, dk, vw, ah, K, ey, uW, uC, ad); #c1(SYNPO2) c2(NP_001122405) c3(synaptopodin-2 isoform b) c4(46796) c5(46797, 46798, 46799, 46800, 46801, 46802) c6( ) c7(ae, ad); #c1(SYNPO2) c2(NP_001122406) c3(synaptopodin-2 isoform c) c4(46803) c5(46804, 46805, 46806, 46807, 46808, 46809) c6( ) c7(ae, ad); #c1(SYNPO2) c2(NP_001273683) c3(synaptopodin-2 isoform d) c4( ) c5(105608) c6( ) c7(ae, ad); #c1(SYNPO2) c2(NP_001273684) c3(synaptopodin-2 isoform e) c4(105609) c5(105610, 105611, 105612, 105613, 105614) c6( ) c7(ae, ad); #c1(SYNPO2) c2(NP_597734) c3(synaptopodin-2 isoform a) c4(235156) c5(235157, 235158, 235159, 235160, 235161, 235162) c6( ) c7(ae, ad); #c1(SYNPR) c2(NP_001123475) c3(synaptoporin isoform 1) c4(47113, 47114) c5(47115, 47116, 47117, 47118, 47119, 47120) c6( ) c7(bM, ac); #c1(SYNPR) c2(NP_653243) c3 (synaptoporin isoform 2 precursor) c4(239289) c5(239290, 239291, 239292, 239293, 239294, 239295, 239296, 239297, 239298, 239299, 239300, 239301) c6( ) c7(bM, ac); #c1(SYP) c2(NP_003170) c3(synaptophysin) c4(135854, 135855) c5(135856, 135857, 135858, 135859, 135860, 135861, 135862, 135863, 135864, 135865, 135866, 135867, 135868, 135869) c6( ) c7(cZ, Tb, Or, aal, ahX, ti, aj, bS, ct, ci, bNe, Fc, iJ, J, xt, aao, ad, iC, ft, lE, ot, nv, WY, pX, SV, iv, h, bK, K, acd, dd, ae, af, Yi, dx, Lg, li, jf, yo, d, bux, s, AQ, anx, ff, nf, Hu, BG, en, g, Z, cm, ek, chr, tj, ac, c, eM, wl, Xu, cJV, xg); #c1(SYP) c2(XP_011542253) c3(synaptophysin isoform X2) c4( ) c5(340787, 340788, 340789, 340790, 340791) c6( ) c7(cZ, Tb, Or, aal, ahX, ti, aj, bS, ct, ci, bNe, Fc, iJ, J, xt, aao, ad, iC, ft, lE, ot, nv, WY, pX, SV, iv, h, bK, K, acl, dd, ae, af, Yi, dx, Lg, li, jf, yo, d, bux, s, AQ, anx, ff, nf, Hu, BG, en, g, Z, cm, ek, chr, tj, ac, c, eM, wl, Xu, cJV, xg); #c1(SYPL1) c2(NP_006745) c3(synaptophysin-like protein 1 isoform a) c4(167633, 167634) c5(167635, 167636, 167637, 167638, 167639, 167640, 167641, 167642, 167643) c6(167644, 167645) c7(ey); #c1(SYPL1) c2(NP_874384) c3(synaptophysin-like protein 1 isoform b) c4( ) c5(256657, 256658, 256659, 256660, 256661) c6( ) c7(ey); #c1(SYPL2) c2(XP_011539585) c3(synaptophysin-like protein 2 isoform X1) c4(337816) c5( ) c6( ) c7(L); #c1(SYPL2) c2(XP_011539587) c3(synaptophysin-like protein 2 isoform X4) c4(337817) c5(337818, 337819, 337820, 337821, 337822, 337823) c6( ) c7(L); #c1(SYT1) c2(NP_001278830) c3(synaptotagmin-1 isoform 2) c4(110677) c5(110678, 110679, 110680, 110681, 110682, 110683, 110684, 110685, 110686, 110687, 110688, 110689, 110690, 110691, 110692, 110693, 110694, 110695, 110696, 110697, 110698, 110699, 110700, 110701) c6( ) c7(oO, bM, T, os, if, al, bEy, eb, fd, lR, ci, bv, aE, K, eG, aW, az, asD, g, eE, cm, bw, axG, nB, cj, iS, m, ht, aj, BF, nA, qN, eW, kH, cU, pi, dx, gi, fe, kp, fq, Qf, dB, cJa, it, B, aA, C, V, q, J, eA, eJ, aSg, yg, aT, ae, af, aAi, jf, nO, Y, r, en, bu, t, f, iK, pb, ck, blC, iM, ac, c, ig, hr, bt, qb, ha, oZ, dG, iJ, ad, XB, dP, hd, azq, hP, ji, azR, gj, d, je, ff, xs, eZ, aF, iV, DF, bF, QC, y, kS, X, brt); #c1(SYT1) c2(NP_005630) c3(synaptotagmin-1 isoform 1) c4(158096, 158097, 158098, 158099, 158100) c5(158101, 158102, 158103, 158104, 158105, 158106, 158107, 158108, 158109, 158110, 158111, 158112, 158113, 158114, 158115, 158116, 158117, 158118, 158119, 158120) c6( ) c7(oO, bM, T, os, if, al, bEy, eb, fd, lR, ci, bv, aE, K, eG, aW, az, asD, g, eE, cm, bw, axG, nB, cj, iS, m, ht, aj, BF, nA, qN, eW, kH, cU, pi, dx, gi, fe, kp, fq, Qf, dB, cJa, it, B, aA, C, V, q, J, eA, eJ, aSg, yg, aT, ae, af, aAi, jf, nO, Y, r, en, bu, t, f, ik, pb, ck, blC, iM, ac, c, ig, hr, bt, qb, ha, oZ, dG, iJ, ad, XB, dP, hd, azq, hP, ji, azR, gj, d, je, ff, xs, eZ, aF, iV, DF, bF, QC, y, kS, X, brt); #c1(SYT11) c2(NP_689493) c3(synaptotagmin-11) c4(242918, 242919) c5(242920, 242921, 242922, 242923, 242924, 242925, 242926, 242927, 242928, 242929, 242930, 242931) c6( ) c7(bL, an); #c1(SYT12) c2(NP_001171351) c3(synaptotagmin-12) c4( ) c5(70055, 70056, 70057, 70058, 70059, 70060, 70061, 70062, 70063, 70064, 70065) c6( ) c7(bL); #c1(SYT12) c2(XP_006718800) c3(synaptotagmin-12 isoform X3) c4( ) c5(292002, 292003) c6( ) c7(bL); #c1(SYT12) c2(XP_011543648) c3(synaptotagmin-12 isoform X2) c4(342763) c5(342764) c6( ) c7(bL); #c1(SYT13) c2(NP_065877) c3(synaptotagmin-13 isoform 1) c4(206042) c5(206043, 206044, 206045, 206046, 206047, 206048, 206049, 206050) c6( ) c7(aq, cu); #c1(SYT14) c2(NP_001139733) c3(synaptotagmin-14 isoform 1) c4(59789) c5(59790, 59791, 59792, 59793, 59794, 59795) c6( ) c7(oe, aw, U, cJW); #c1(SYT14) c2(NP_001139734) c3(synaptotagmin-14 isoform 3) c4(59796, 59797) c5(59798, 59799, 59800, 59801, 59802, 59803, 59804, 59805, 59806, 59807, 59808, 59809, 59810) c6( ) c7(oe, aw, U, cJW); #c1(SYT14) c2(NP_001139736) c3(synaptotagmin-14 isoform 2) c4(59811) c5(59812, 59813, 59814, 59815, 59816, 59817, 59818, 59819, 59820, 59821) c6( ) c7(oe, aw, U, cJW); #c1(SYT14) c2(NP_694994) c3(synaptotagmin-14 isoform 4) c4(245607, 245608) c5(245609, 245610, 245611, 245612, 245613, 245614, 245615, 245616, 245617, 245618, 245619) c6( ) c7(oe, aw, U, cJW); #c1(SYT4) c2(NP_065834) c3(synaptotagmin-4) c4(205752, 205753) c5(205754, 205755, 205756, 205757, 205758, 205759, 205760, 205761, 205762, 205763, 205764, 205765) c6( ) c7(bL, he, Y, fe); #c1(SYT6) c2(XP_011539054) c3(synaptotagmin-6 isoform X2) c4( ) c5(337125, 337126, 337127, 337128, 337129, 337130, 337131, 337132, 337133, 337134, 337135, 337136) c6( ) c7(he, fr); #c1(SYT9) c2(NP_783860) c3(synaptotagmin-9) c4(251764) c5(251765, 251766, 251767, 251768, 251769, 251770) c6( ) c7(aE); #c1(SYT9) c2(XP_011518204) c3(synaptotagmin-9 isoform X3) c4(312239) c5(312240, 312241, 312242, 312243, 312244, 312245) c6( ) c7(aE); #c1(SYTL1) c2(XP_011540618) c3(synaptotagmin-like protein 1 isoform X4) c4(339146) c5(339147, 339148, 339149, 339150, 339151, 339152) c6( ) c7(h, ae, ci); #c1(SYTL2) c2(NP_001156423) c3(synaptotagmin-like protein 2 isoform g) c4(61712) c5(61713, 61714, 61715, 61716, 61717, 61718, 61719) c6( ) c7(T, AM, ff, km, aA, io, it, aF, K, en, hB, bz); #c1(SYTL2) c2(NP_001156424) c3(synaptotagmin-like protein 2 isoform h) c4( ) c5(61720, 61721, 61722, 61723, 61724, 61725, 61726, 61727, 61728, 61729) c6( ) c7(T, AM, ff, km, aA, io, it, aF, K, en, hB, bz); #c1(SYTL2) c2(NP_001156425) c3(synaptotagmin-like protein 2 isoform i) c4(61730, 61731) c5(61732, 61733, 61734, 61735, 61736, 61737) c6( ) c7(T, AM, ff, km, aA, io, it, aF, K, en, hB, bz); #c1(SYTL2) c2(NP_001276537) c3(synaptotagmin-like protein 2 isoform j) c4( ) c5(108215, 108216, 108217, 108218, 108219, 108220, 108221, 108222, 108223, 108224) c6( ) c7(T, AM, ff, km, aA, io, it, aF, K, en, hB, bz); #c1(SYTL2) c2(NP_116561) c3(synaptotagmin-like protein 2 isoform a) c4(226179) c5(226180, 226181, 226182, 226183, 226184, 226185, 226186, 226187, 226188, 226189, 226190, 226191) c6( ) c7(T, AM, ff, km, aA, io, it, aF, K, en, hB, bz); #c1(SYTL2) c2(NP_996812) c3(synaptotagmin-like protein 2 isoform b) c4( ) c5(264612, 264613, 264614, 264615, 264616, 264617, 264618, 264619, 264620, 264621, 264622, 264623, 264624, 264625, 264626, 264627, 264628, 264629, 264630, 264631, 264632, 264633, 264634, 264635, 264636, 264637, 264638, 264639) c6( ) c7(T, AM, ff, km, aA, io, it, aF, K, en, hB, bz); #c1(SYTL2) c2(NP_996813) c3 (synaptotagmin-like protein 2 isoform f) c4( ) c5(264640, 264641, 264642, 264643, 264644, 264645) c6( ) c7(T, AM, ff, km, aA, io, it, aF, K, en, hB, bz); #c1(SYTL2) c2(XP_005274114) c3(synaptotagmin-like protein 2 isoform X5) c4(284946, 284947, 284948, 284949) c5(284950, 284951, 284952, 284953, 284954, 284955, 284956, 284957, 284958, 284959, 284960, 284961) c6( ) c7(T, AM, ff, km, aA, io, it, aF, K, en, hB, bz); #c1(SYTL2) c2(XP_005274118) c3(synaptotagmin-like protein 2 isoform X13) c4(284962) c5(284963) c6( ) c7(T, AM, ff, km, aA, io, it, aF, K, en, hB, bz); #c1(SYTL2) c2(XP_011543408) c3(synaptotagmin-like protein 2 isoform X4) c4( ) c5(342299, 342300) c6( ) c7(T, AM, ff, km, aA, io, it, aF, K, en, hB, bz); #c1(SYTL2) c2(XP_011543409) c3 (synaptotagmin-like protein 2 isoform X6) c4(342301, 342302) c5( ) c6( ) c7(T, AM, ff, km, aA, io, it, aF, K, en, hB, bz); #c1(SYTL2) c2(XP_011543410) c3(synaptotagmin-like protein 2 isoform X7) c4(342303, 342304, 342305) c5(342306, 342307, 342308, 342309, 342310, 342311) c6( ) c7(T, AM, ff, km, aA, io, it, aF, K, en, hB, bz); #c1(SYTL2) c2(XP_011543411) c3(synaptotagmin-like protein 2 isoform X8) c4(342312) c5(342313, 342314, 342315, 342316, 342317, 342318) c6( ) c7(T, AM, ff, km, aA, io, it, aF, K, en, hB, bz); #c1(SYTL2) c2(XP_011543412) c3(synaptotagmin-like protein 2 isoform X9) c4(342319) c5( ) c6( ) c7(T, AM, ff, km, aA, io, it, aF, K, en, hB, bz); #c1(SYTL5) c2(NP_001156806) c3(synaptotagmin-like protein 5 isoform 2) c4(61983) c5(61984, 61985, 61986, 61987, 61988, 61989) c6( ) c7(iX); #c1(SYTL5) c2(NP_001156807) c3(synaptotagmin-like protein 5 isoform 1) c4(61990) c5(61991, 61992, 61993, 61994, 61995, 61996, 61997, 61998, 61999, 62000) c6( ) c7(iX); #c1(SYVN1) c2(NP_115807) c3(E3 ubiquitin-protein ligase synoviolin isoform a precursor) c4(223979, 223980) c5(223981, 223982, 223983, 223984, 223985, 223986, 223987, 223988, 223989, 223990, 223991, 223992, 223993, 223994, 223995, 223996, 223997, 223998, 223999, 224000, 224001, 224002, 224003, 224004, 224005, 224006, 224007, 224008, 224009) c6( ) c7(T, RX, al, oA, aOH, jh, fr, iJ, xE, rw, jZ, K, mR, ff, en, bw, avK, es, iA, Jr, km, Kg, y); #c1(SYVN1) c2(NP_757385) c3(E3 ubiquitin-protein ligase synoviolin isoform b precursor) c4(249106, 249107) c5( ) c6( ) c7(T, RX, al, oA, aOH, jh, fr, iJ, xE, rw, jZ, K, mR, ff, en, bw, avK, es, iA, Jr, km, Kg, y); #c1(T) c2(NP_001257413) c3(brachyury protein isoform 2) c4(95376, 95377) c5(95378, 95379, 95380, 95381, 95382, 95383, 95384, 95385, 95386, 95387, 95388, 95389) c6( ) c7(cJY, cJX, ff, ci, en, ik); #c1(T) c2(NP_003172) c3(brachyury protein isoform 1) c4(135870) c5(135871, 135872, 135873, 135874, 135875, 135876, 135877) c6( ) c7(cJY, cJX, ff, ci, en, ik); #c1(TAAR1) c2(NP_612200) c3(trace amine-associated receptor 1) c4(236189) c5(236190, 236191, 236192, 236193, 236194, 236195) c6( ) c7(bEV, fy, xg); #c1(TAAR2) c2(NP_001028252) c3(trace amine-associated receptor 2 isoform 1) c4(30414, 30415) c5(30416, 30417, 30418, 30419, 30420, 30421, 30422) c6( ) c7(h); #c1(TAAR2) c2(NP_055441) c3(trace amine-associated receptor 2 isoform 2) c4( ) c5(181158, 181159, 181160, 181161, 181162, 181163, 181164, 181165) c6( ) c7(h); #c1(TAAR5) c2(NP_003958) c3(trace amine-associated receptor 5) c4(142458) c5(142459, 142460, 142461, 142462, 142463, 142464) c6( ) c7(bFo, cll); #c1(TAAR6) c2(NP_778237) c3(trace amine-associated receptor 6) c4(251491, 251492) c5(251493, 251494, 251495, 251496, 251497, 251498, 251499, 251500, 251501, 251502, 251503, 251504) c6( ) c7(Z, bS, bK, ct, bO); #c1(TAB1) c2(NP_006107) c3(TGF-beta-activated kinase 1 and MAP3K7-binding protein 1 isoform alpha) c4(161901) c5(161902, 161903, 161904, 161905, 161906, 161907) c6( ) c7(bM, kl, y, aT); #c1(TAB1) c2(NP_705717) c3(TGF-beta-activated kinase 1 and MAP3K7-binding protein 1 isoform beta) c4(246275) c5(246276, 246277, 246278, 246279, 246280, 246281) c6( ) c7(bM, kl, y, aT); #c1(TAB2) c2(NP_001278964) c3(TGF-beta-activated kinase 1 and MAP3K7-binding protein 2 isoform b) c4(111149) c5(111150, 111151, 111152, 111153, 111154, 111155) c6( ) c7(md, A, bjA, aZ, aW, au, aw, aT, hD, cJZ, ci, bD, y); #c1(TAB2) c2(NP_055908) c3(TGF-beta-activated kinase 1 and MAP3K7-binding protein 2 isoform a) c4(184422, 184423) c5(184424, 184425, 184426, 184427, 184428, 184429, 184430, 184431, 184432, 184433, 184434, 184435, 184436, 184437, 184438, 184439, 184440, 184441, 184442, 184443) c6( ) c7(md, A, bjA, aZ, aW, au, aw, aT, hD, cJZ, ci, bD, y); #c1(TAB3) c2(XP_005274539) c3(TGF-beta-activated kinase 1 and MAP3K7-binding protein 3 isoform X1) c4(285128) c5(285129, 285130, 285131, 285132, 285133, 285134, 285135, 285136, 285137, 285138, 285139, 285140, 285141, 285142) c6( ) c7(nD, c, af, cr, n, tv, tu, C, B, amL); #c1(TAB3) c2(XP_005274540) c3(TGF-beta-activated kinase 1 and MAP3K7-binding protein 3 isoform X3) c4(285143) c5(285144, 285145, 285146, 285147, 285148, 285149) c6( ) c7(nD, c, af, cr, n, tv, tu, C, B, amL); #c1(TAB3) c2(XP_011543790) c3(TGF-beta-activated kinase 1 and MAP3K7-binding protein 3 isoform X5) c4(342890) c5(342891, 342892, 342893, 342894, 342895, 342896) c6( ) c7(nD, c, af, cr, n, tv, tu, C, B, amL); #c1(TAC1) c2(NP_003173) c3(protachykinin-1 isoform beta precursor) c4(135878) c5(135879, 135880, 135881, 135882, 135883, 135884) c6( ) c7(bM, T, vS, pf, ea, bO, ci, Jx, x, xy, aE, fS, Ds, abj, K, GO, hu, UO, aW, mQ, as, hJ, eE, cm, y, eM, JA, Ji, U, k, Dv, cE, rt, cZ, dD, n, To, aj, oY, ct, cd, jz, cU, h, wb, dd, he, cka, asp, bux, cly, sp, fe, pm, vo, hq, cA, w, at, uz, pg, YH, it, Dw, wL, aA, v, j, V, eA, J, cF, DK, aT, dz, bK, UN, ae, af, Y, fO, la, r, lo, tu, iK, rY, aHi, ck, Qi, Ta, sS, bN, ac, c, yW, bQC, dh, auT, mf, qX, df, Hn, biV, bS, eq, Fb, ad, dP, Tg, WR, lC, vB, ji, uQ, cr, tv, EZ, iV, Z, bz, bF, zr, HP, dJ, rH, lf, ra, jr, Tc); #c1(TAC1) c2(NP_054702) c3 (protachykinin-1 isoform alpha precursor) c4(177307) c5(177308, 177309, 177310, 177311, 177312, 177313) c6( ) c7(bM, T, vS, pf, ea, bO, ci, Jx, x, xy, aE, fS, Ds, abj, K, GO, hu, UO, aW, mQ, as, hJ, eE, cm, y, eM, JA, Ji, U, k, Dv, cE, rt, cZ, dD, n, To, aj, oY, ct, cd, jz, cU, h, wb, dd, he, cKa, asp, bux, cly, sp, fe, pm, vo, hq, cA, w, at, uz, pg, YH, it, Dw, wL, aA, v, j, V, eA, J, cF, DK, aT, dz, bK, UN, ae, af, Y, fO, la, r, lo, tu, ik, rY, aHi, ck, Qi, Ta, sS, bN, ac, c, yW, bQC, dh, auT, mf, qX, df, Hn, biV, bS, eq, Fb, ad, dP, Tg, WR, lC, vB, ji, uQ, cr, tv, EZ, iV, Z, bz, bF, zr, HP, dJ, rH, lf, ra, jr, Tc); #c1(TAC1) c2(NP_054703) c3(protachykinin-1 isoform gamma precursor) c4(177314) c5(177315, 177316, 177317, 177318, 177319, 177320) c6( ) c7(bM, T, vS, pf, ea, bO, ci, Jx, x, xy, aE, fS, Ds, abj, K, GO, hu, UO, aW, mQ, as, hJ, eE, cm, y, eM, JA, Ji, U, k, Dv, cE, rt, cZ, dD, n, To, aj, oY, ct, cd, jz, cU, h, wb, dd, he, cKa, asp, bux, cly, sp, fe, pm, vo, hq, cA, w, at, uz, pg, YH, it, Dw, wL, aA, v, j, V, eA, J, cF, DK, aT, dz, bK, UN, ae, af, Y, fO, la, r, lo, tu, iK, rY, aHi, ck, Qi, Ta, sS, bN, ac, c, yW, bQC, dh, auT, mf, qX, df, Hn, biV, bS, eq, Fb, ad, dP, Tg, WR, lC, vB, ji, uQ, cr, tv, EZ, iV, Z, bz, bF, zr, HP, dJ, rH, lf, ra, jr, Tc); #c1(TAC3) c2(NP_001171525) c3(tachykinin-3 isoform 2 preproprotein) c4(70449) c5(70450, 70451, 70452, 70453, 70454, 70455, 70456, 70457) c6( ) c7(bM, aMP, yi, vA, cz, eb, K, UF, uC, V, cKb, op, UE); #c1(TAC3) c2(XP_011537013) c3(tachykinin-3 isoform X1) c4(334752) c5(334753, 334754, 334755, 334756, 334757, 334758, 334759, 334760, 334761, 334762, 334763, 334764, 334765, 334766, 334767, 334768, 334769, 334770, 334771, 334772) c6( ) c7(bM, aMP, yi, vA, cz, eb, K, UF, uC, V, cKb, op, UE); #c1(TAC4) c2(NP_001070971) c3(tachykinin-4 isoform beta precursor) c4(35119) c5(35120, 35121, 35122, 35123, 35124, 35125, 35126) c6(35127) c7(bM, V, eA); #c1(TAC4) c2(NP_001070972) c3(tachykinin-4 isoform gamma precursor) c4(35128) c5(35129, 35130, 35131, 35132, 35133, 35134, 35135) c6(35136) c7(bM, V, eA); #c1(TAC4) c2(NP_001070973) c3(tachykinin-4 isoform delta precursor) c4(35137) c5(35138, 35139, 35140, 35141, 35142, 35143, 35144) c6(35145) c7(bM, V, eA); #c1(TAC4) c2(NP_001070974) c3(tachykinin-4 isoform alpha-2 precursor) c4(35146) c5(35147, 35148, 35149, 35150, 35151, 35152, 35153, 35154) c6(35155, 35156) c7(bM, V, eA); #c1(TAC4) c2(NP_733786) c3(tachykinin-4 isoform alpha precursor) c4(247089, 247090) c5(247091, 247092, 247093, 247094, 247095, 247096, 247097, 247098, 247099, 247100, 247101, 247102, 247103, 247104, 247105) c6(247106, 247107, 247108) c7(bM, V, eA); #c1(TACC1) c2(NP_001116296) c3(transforming acidic coiled-coil-containing protein 1 isoform 2) c4(43725) c5(43726, 43727, 43728, 43729, 43730, 43731) c6( ) c7(aW, aF, aA, aT); #c1(TACC1) c2(NP_001139688) c3(transforming acidic coiled-coil-containing protein 1 isoform 3) c4(59723, 59724, 59725, 59726, 59727) c5(59728, 59729, 59730, 59731, 59732, 59733, 59734, 59735, 59736, 59737, 59738, 59739, 59740, 59741, 59742) c6( ) c7(aW, aF, aA, aT); #c1(TACC1) c2(NP_006274) c3(transforming acidic coiled-coil-containing protein 1 isoform 1) c4(163490) c5(163491, 163492, 163493, 163494, 163495, 163496, 163497, 163498, 163499, 163500, 163501, 163502, 163503) c6( ) c7(aW, aF, aA, aT); #c1(TACC1) c2(XP_011542934) c3 (transforming acidic coiled-coil-containing protein 1 isoform X1) c4(341580) c5(341581, 341582, 341583, 341584, 341585, 341586, 341587, 341588, 341589, 341590, 341591) c6( ) c7(aW, aF, aA, al); #c1(TACC1) c2(XP_011542936) c3(transforming acidic coiled-coil-containing protein 1 isoform X5) c4(341592, 341593, 341594, 341595) c5(341596, 341597, 341598, 341599, 341600, 341601, 341602, 341603, 341604, 341605, 341606, 341607, 341608, 341609, 341610, 341611, 341612, 341613, 341614, 341615, 341616, 341617, 341618, 341619, 341620, 341621, 341622) c6( ) c7(aW, aF, aA, aT); #c1(TACC1) c2(XP_011542938) c3(transforming acidic coiled-coil-containing protein 1 isoform X7) c4(341623, 341624) c5(341625, 341626, 341627, 341628, 341629, 341630, 341631, 341632, 341633, 341634, 341635, 341636) c6( ) c7(aW, aF, aA, aT); #c1(TACC2) c2(NP_001278805) c3(transforming acidic coiled-coil-containing protein 2 isoform e) c4( ) c5(110139) c6( ) c7(aW, ae, ad, aT); #c1(TACC2) c2(NP_001278806) c3(transforming acidic coiled-coil-containing protein 2 isoform f) c4(110140) c5(110141, 110142, 110143, 110144, 110145, 110146) c6( ) c7(aW, ae, ad, aT); #c1(TACC2) c2(NP_008928) c3(transforming acidic coiled-coil-containing protein 2 isoform d) c4(169606) c5(169607, 169608, 169609, 169610, 169611, 169612) c6( ) c7(aW, ae, ad, aT); #c1(TACC2) c2(NP_996742) c3(transforming acidic coiled-coil-containing protein 2 isoform c) c4(264395) c5(264396, 264397, 264398, 264399, 264400, 264401) c6( ) c7(aW, ae, ad, aT); #c1(TACC2) c2(NP_996743) c3(transforming acidic coiled-coil-containing protein 2 isoform b) c4(264402) c5(264403, 264404, 264405, 264406, 264407, 264408, 264409, 264410, 264411, 264412) c6( ) c7(aW, ae, ad, aT); #c1(TACC2) c2(NP_996744) c3(transforming acidic coiled-coil-containing protein 2 isoform a) c4(264413) c5(264414, 264415, 264416, 264417, 264418, 264419, 264420, 264421, 264422, 264423) c6( ) c7(aW, ae, ad, aT); #c1(TACC2) c2(XP_006717616) c3(transforming acidic coiled-coil-containing protein 2 isoform X15) c4(291250) c5(291251, 291252, 291253, 291254, 291255, 291256) c6( ) c7(aW, ae, ad, aT); #c1(TACC2) c2(XP_006717617) c3(transforming acidic coiled-coil-containing protein 2 isoform X16) c4(291257) c5(291258, 291259, 291260, 291261, 291262) c6( ) c7(aW, ae, ad, aT); #c1(TACC3) c2(NP_006333) c3(transforming acidic coiled-coil-containing protein 3) c4(164094, 164095) c5(164096, 164097, 164098, 164099, 164100, 164101) c6( ) c7(T, oS, id, d, Qu, if, jh, aT, fd, dL, qN, eL, eA); #c1(TACC3) c2(XP_005247986) c3(transforming acidic coiled-coil-containing protein 3 isoform X1) c4(268163) c5( ) c6( ) c7(T, oS, id, d, Qu, if, jh, aT, fd, dL, qN, eL, eA); #c1(TACC3) c2(XP_005247987) c3(transforming acidic coiled-coil-containing protein 3 isoform X4) c4(268164) c5(268165, 268166, 268167, 268168, 268169, 268170, 268171) c6( ) c7(T, oS, id, d, Qu, if, jh, aT, fd, dL, qN, eL, eA); #c1(TACC3) c2(XP_011511689) c3(transforming acidic coiled-coil-containing protein 3 isoform X3) c4( ) c5(302418, 302419, 302420, 302421, 302422, 302423, 302424, 302425, 302426, 302427, 302428, 302429) c6( ) c7(T, oS, id, d, Qu, if, jh, aT, fd, dL, qN, eL, eA); #c1(TACO1) c2(NP_057444) c3(translational activator of cytochrome c oxidase 1) c4(190923) c5(190924, 190925, 190926, 190927, 190928, 190929) c6( ) c7(bZ, aUx, acJ, ajU); #c1(TACR1) c2(NP_001049) c3(substance-P receptor isoform long) c4(34377, 34378) c5(34379, 34380, 34381, 34382, 34383, 34384) c6( ) c7(bM, T, jv, qX, vS, cz, n, Gd, ww, ci, eA, ad, dP, x, aji, aE, bWj, WR, aT, cU, h, bK, UN, K, bah, ji, aCU, on, he, ae, akl, uQ, tn, d, tb, aW, sp, EU, Y, fe, tv, lo, tu, as, iV, pm, f, cM, iK, hq, ck, ar, Ta, bP, c, cA, Ei, k, EY, YH, yA, lC, cE, y, Tc); #c1(TACR1) c2(NP_056542) c3(substance-P receptor isoform short) c4( ) c5(188082, 188083, 188084, 188085, 188086, 188087) c6( ) c7(bM, T, jv, qX, vS, cz, n, Gd, ww, ci, eA, ad, dP, x, aji, aE, bWj, WR, aT, cU, h, bK, UN, K, bah, ji, aCU, on, he, ae, akl, uQ, tn, d, tb, aW, sp, EU, Y, fe, tv, lo, tu, as, iV, pm, f, cM, iK, hq, ck, ar, Ta, bP, c, cA, Ei, k, EY, YH, yA, lC, cE, y, Tc); #c1(TACR2) c2(NP_001048) c3(substance-K receptor) c4(34363, 34364) c5(34365, 34366, 34367, 34368, 34369, 34370, 34371, 34372, 34373, 34374, 34375, 34376) c6( ) c7(cw, bM, Dw, tn, ac, at, fe, Ds, ji, K, bah, iV, lC, blu); #c1(TACR3) c2(NP_001050) c3(neuromedin-K receptor) c4(34385) c5(34386, 34387, 34388, 34389, 34390, 34391) c6( ) c7(bL, bM, Dw, cz, bBX, eb, wV, bS, ct, bWN, UE, bWj, h, Ds, yi, cu, lo, Z, op, aMP, AV, vA, Gx, UF, y, cKc); #c1(TADA1) c2(NP_444281) c3(transcriptional adapter 1) c4(230281) c5(230282, 230283, 230284, 230285, 230286, 230287) c6( ) c7(bL, A, cz, tV, j, y, bA); #c1(TADA2A) c2(NP_001159577) c3(transcriptional adapter 2-alpha isoform a) c4(64323) c5(64324, 64325, 64326, 64327, 64328, 64329, 64330, 64331, 64332, 64333, 64334, 64335, 64336, 64337, 64338, 64339) c6( ) c7(y, j, ac, dV); #c1(TADA2A) c2(NP_001278847) c3(transcriptional adapter 2-alpha isoform b) c4(110713) c5(110714, 110715, 110716, 110717, 110718, 110719) c6( ) c7(y, j, ac, dV); #c1(TADA2A) c2(XP_011523456) c3(transcriptional adapter 2-alpha isoform X1) c4(317977) c5( ) c6( ) c7(y, j, ac, dV); #c1(TADA2A) c2(XP_011523458) c3(transcriptional adapter 2-alpha isoform X3) c4( ) c5(317978, 317979, 317980, 317981, 317982) c6( ) c7(y, j, ac, dV); #c1(TADA3) c2(NP_006345) c3(transcriptional adapter 3 isoform a) c4(164194, 164195, 164196) c5(164197, 164198, 164199, 164200, 164201, 164202, 164203, 164204, 164205, 164206, 164207) c6( ) c7(aW, aT); #c1(TADA3) c2(NP_597814) c3(transcriptional adapter 3 isoform b) c4( ) c5(235197, 235198, 235199, 235200, 235201, 235202, 235203, 235204, 235205, 235206, 235207, 235208) c6( ) c7(aW, aT); #c1(TAF1) c2(NP_001273003) c3(transcription initiation factor TFIID subunit 1 isoform 3) c4(104143, 104144) c5(104145, 104146, 104147, 104148, 104149, 104150) c6( ) c7(qJ, ae, T, hd, nB, oe, bps, aep, Yk, akt, aU, pD, j, cm, aVc, af, ad); #c1(TAF1) c2(NP_004597) c3(transcription initiation factor TFIID subunit 1 isoform 1) c4(148020) c5(148021, 148022, 148023, 148024, 148025, 148026, 148027) c6( ) c7(qJ, ae, T, hd, nB, oe, bps, aep, Yk, akt, aU, pD, j, cm, aVc, af, ad); #c1(TAF1) c2(NP_620278) c3(transcription initiation factor TFIID subunit 1 isoform 2) c4(237492) c5(237493, 237494, 237495, 237496, 237497, 237498, 237499) c6( ) c7(qJ, ae, T, hd, nB, oe, bps, aep, Yk, akt, aU, pD, j, cm, aVc, af, ad); #c1(TAF1) c2(XP_005262352) c3(transcription initiation factor TFIID subunit 1 isoform X1) c4( ) c5(277846) c6( ) c7(qJ, ae, T, hd, nB, oe, bps, aep, Yk, akt, aU, pD, j, cm, aVc, af, ad); #c1(TAF1) c2(XP_005262354) c3(transcription initiation factor TFIID subunit 1 isoform X3) c4(277847) c5(277848, 277849, 277850, 277851, 277852, 277853) c6( ) c7(qJ, ae, T, hd, nB, oe, bps, aep, Yk, akt, aU, pD, j, cm, aVc, af, ad); #c1(TAF1) c2(XP_006724745) c3(transcription initiation factor TFIID subunit 1 isoform X5) c4(296298) c5(296299, 296300, 296301, 296302, 296303, 296304, 296305, 296306, 296307, 296308) c6( ) c7(qJ, ae, T, hd, nB, oe, bps, aep, Yk, akt, aU, pD, j, cm, aVc, af, ad); #c1(TAF15) c2(NP_631961) c3(TATA-binding protein-associated factor 2N isoform 1) c4(238416, 238417) c5( ) c6( ) c7(ck, T, kh, aNA, QG, c, WY, au, aA, aaE, PJ, eF, bt, ae, ad); #c1(TAF15) c2(XP_011523617) c3(TATA-binding protein-associated factor 2N isoform X2) c4( ) c5(318090, 318091, 318092, 318093, 318094, 318095, 318096, 318097, 318098, 318099, 318100, 318101, 318102, 318103, 318104, 318105, 318106) c6( ) c7(ck, T, kh, aNA, QG, c, WY, au, aA, aaE, PJ, eF, bt, ae, ad); #c1(TAF1B) c2(NP_005671) c3(TATA box-binding protein-associated factor RNA polymerase I subunit B) c4(158421) c5(158422, 158423, 158424, 158425, 158426, 158427, 158428) c6( ) c7(q); #c1(TAF1B) c2(XP_011508713) c3(TATA box-binding protein-associated factor RNA polymerase I subunit B isoform X2) c4(299319) c5(299320) c6( ) c7(q); #c1(TAF1B) c2(XP_011508715) c3(TATA box-binding protein-associated factor RNA polymerase I subunit B isoform X3) c4( ) c5(299321, 299322, 299323, 299324, 299325) c6( ) c7(q); #c1(TAF1C) c2(NP_001230089) c3(TATA box-binding protein-associated factor RNA polymerase I subunit C isoform 6) c4( ) c5(85895, 85896, 85897, 85898, 85899) c6( ) c7(bN); #c1(TAF1C) c2(NP_647610) c3(TATA box-binding protein-associated factor RNA polymerase I subunit C isoform 2) c4(238797, 238798) c5(238799, 238800, 238801, 238802, 238803) c6( ) c7(bN); #c1(TAF1C) c2(XP_005256283) c3(TATA box-binding protein-associated factor RNA polymerase I subunit C isoform X1) c4(274053) c5(274054, 274055, 274056, 274057, 274058, 274059, 274060, 274061, 274062, 274063, 274064) c6( ) c7(bN); #c1(TAF1C) c2(XP_006721389) c3(TATA box-binding protein-associated factor RNA polymerase I subunit C isoform X3) c4(293955, 293956) c5(293957, 293958, 293959, 293960, 293961, 293962, 293963, 293964, 293965, 293966, 293967, 293968, 293969, 293970) c6( ) c7(bN); #c1(TAF1D) c2(NP_077021) c3(TATA box-binding protein-associated factor RNA polymerase I subunit D) c4(214760) c5(214761, 214762, 214763, 214764, 214765, 214766, 214767, 214768, 214769, 214770, 214771, 214772, 214773, 214774) c6( ) c7(Pk, lQ); #c1(TAF2) c2(NP_003175) c3(transcription initiation factor TFIID subunit 2) c4(135900) c5(135901, 135902, 135903, 135904, 135905, 135906, 135907) c6( ) c7(cKd, rU, U, CQ, nA, cm); #c1(TAF3) c2(NP_114129) c3(transcription initiation factor TFIID subunit 3) c4(222285, 222286) c5(222287, 222288, 222289, 222290, 222291, 222292, 222293, 222294, 222295, 222296, 222297) c6( ) c7(ac, abV, aw, e, j, eu); #c1(TAF3) c2(XP_011518043) c3(transcription initiation factor TFIID subunit 3 isoform X1) c4( ) c5(312052, 312053, 312054) c6( ) c7(ac, abV, aw, e, j, eu); #c1(TAF4) c2(NP_003176) c3(transcription initiation factor TFIID subunit 4) c4(135908, 135909) c5(135910, 135911, 135912, 135913, 135914, 135915, 135916, 135917, 135918, 135919, 135920, 135921, 135922, 135923, 135924) c6( ) c7(on, ac); #c1(TAF4B) c2(NP_001280654) c3(transcription initiation factor TFIID subunit 4B isoform 1) c4(111565) c5(111566, 111567, 111568, 111569, 111570, 111571, 111572) c6( ) c7(il, T, ci, ev); #c1(TAF4B) c2(NP_005631) c3(transcription initiation factor TFIID subunit 4B isoform 2) c4(158121, 158122) c5(158123, 158124, 158125, 158126, 158127, 158128) c6( ) c7(il, T, ci, ev); #c1(TAF4B) c2(XP_005258396) c3(transcription initiation factor TFIID subunit 4B isoform X2) c4( ) c5(275642, 275643, 275644, 275645, 275646, 275647) c6( ) c7(il, T, ci, ev); #c1(TAF5L) c2(NP_001020418) c3(TAF5-like RNA polymerase II p300/CBP-associated factor-associated factor 65 kDa subunit 5L isoform b) c4(28753, 28754) c5(28755, 28756, 28757, 28758, 28759, 28760, 28761, 28762, 28763, 28764, 28765, 28766) c6( ) c7(ac, A, aj, J, aT); #c1(TAF5L) c2(NP_055224) c3(TAF5-like RNA polymerase II p300/CBP-associated factor-associated factor 65 kDa subunit 5L isoform a) c4(179926) c5(179927, 179928, 179929, 179930, 179931, 179932, 179933, 179934, 179935, 179936) c6( ) c7(ac, A, aj, J, aT); #c1(TAF6) c2(NP_001177344) c3 (transcription initiation factor TFIID subunit 6 isoform epsilon) c4(72663, 72664, 72665, 72666, 72667) c5(72668, 72669, 72670, 72671, 72672, 72673, 72674, 72675, 72676, 72677, 72678, 72679, 72680, 72681, 72682, 72683, 72684, 72685, 72686, 72687, 72688, 72689, 72690, 72691, 72692, 72693) c6( ) c7(aNL); #c1(TAF6) c2(NP_005632) c3(transcription initiation factor TFIID subunit 6 isoform alpha) c4(158129, 158130, 158131, 158132) c5(158133, 158134, 158135, 158136, 158137, 158138, 158139, 158140, 158141, 158142, 158143, 158144, 158145, 158146, 158147, 158148, 158149, 158150, 158151, 158152, 158153, 158154, 158155, 158156, 158157, 158158, 158159, 158160, 158161, 158162, 158163, 158164, 158165, 158166, 158167, 158168, 158169, 158170, 158171, 158172, 158173) c6( ) c7(aNL); #c1(TAF7) c2(NP_005633) c3(transcription initiation factor TFIID subunit 7) c4(158174) c5(158175, 158176, 158177, 158178, 158179, 158180, 158181) c6( ) c7(bM); #c1(TAF7L) c2(NP_001161946) c3(transcription initiation factor TFIID subunit 7-like isoform 2) c4( ) c5(66304, 66305, 66306, 66307, 66308) c6( ) c7(gU); #c1(TAF7L) c2(NP_079161) c3(transcription initiation factor TFIID subunit 7-like isoform 1) c4(217275, 217276) c5(217277, 217278, 217279, 217280, 217281, 217282, 217283) c6( ) c7(gU); #c1(TAF7L) c2(XP_005262208) c3(transcription initiation factor TFIID subunit 7-like isoform X2) c4(277774) c5(277775, 277776, 277777, 277778, 277779, 277780, 277781, 277782, 277783, 277784) c6( ) c7(gU); #c1(TAF8) c2(NP_612639) c3(transcription initiation factor TFIID subunit 8) c4(236729, 236730) c5(236731, 236732, 236733, 236734, 236735, 236736) c6( ) c7(af, aT, kH); #c1(TAF8) c2(XP_005248906) c3(transcription initiation factor TFIID subunit 8 isoform X3) c4( ) c5(268776, 268777, 268778, 268779, 268780, 268781) c6( ) c7(af, aT, kH); #c1(TAF8) c2(XP_011512598) c3(transcription initiation factor TFIID subunit 8 isoform X1) c4(303501) c5(303502, 303503, 303504, 303505, 303506, 303507, 303508, 303509, 303510, 303511) c6( ) c7(af, aT, kH); #c1(TAF8) c2(XP_011512599) c3(transcription initiation factor TFIID subunit 8 isoform X2) c4(303512) c5(303513, 303514, 303515, 303516, 303517, 303518) c6( ) c7(af, aT, kH); #c1(TAF9) c2(NP_003178) c3(transcription initiation factor TFIID subunit 9) c4(135925, 135926, 135927, 135928, 135929, 135930) c5(135931, 135932, 135933, 135934, 135935, 135936, 135937, 135938, 135939, 135940, 135941, 135942, 135943, 135944, 135945, 135946, 135947, 135948, 135949, 135950, 135951, 135952, 135953, 135954, 135955, 135956, 135957, 135958, 135959, 135960, 135961, 135962, 135963, 135964, 135965, 135966, 135967, 135968, 135969, 135970, 135971, 135972, 135973, 135974, 135975, 135976, 135977, 135978, 135979) c6( ) c7(h, iu, ey, B); #c1(TAGAP) c2(NP_001265662) c3(T-cell activation Rho GTPase-activating protein isoform d) c4( ) c5(99182, 99183, 99184, 99185, 99186, 99187) c6( ) c7(A, ju, cr, cKe, cL, hp, C, iV, y, V); #c1(TAGAP) c2(NP_473455) c3(T-cell activation Rho GTPase-activating protein isoform b) c4(230553, 230554, 230555) c5(230556, 230557, 230558, 230559, 230560, 230561) c6( ) c7(A, ju, cr, cKe, cL, hp, C, iV, y, V); #c1(TAGAP) c2(NP_620165) c3(T-cell activation Rho GTPase-activating protein isoform c) c4(237437) c5(237438, 237439, 237440, 237441, 237442, 237443) c6( ) c7(A, ju, cr, cKe, cL, hp, C, iV, y, V); #c1(TAGAP) c2(NP_687034) c3(T-cell activation Rho GTPase-activating protein isoform a) c4( ) c5(242651, 242652, 242653, 242654, 242655, 242656) c6( ) c7(A, ju, cr, cKe, cL, hp, C, iV, y, V); #c1(TAGLN) c2(NP_001001522) c3(transgelin) c4(18566, 18567, 18568) c5(18569, 18570, 18571, 18572, 18573, 18574, 18575, 18576, 18577, 18578, 18579, 18580, 18581, 18582, 18583, 18584, 18585, 18586, 18587, 18588, 18589, 18590, 18591, 18592, 18593, 18594, 18595, 18596, 18597, 18598, 18599, 18600, 18601) c6( ) c7(ng, bM, T, dD, aC, ea, fG, e, aA, bB, vr, aj, v, eq, ci, q, J, ad, dP, iu, aE, aT, K, ae, B, bf, anP, wr, r, aF, g, bz, pb, LB, eM, bE, DW, dy, fD, du, lC, it, X); #c1(TAGLN2) c2(NP_001264153) c3(transgelin-2 isoform a) c4(97582, 97583, 97584) c5(97585, 97586, 97587, 97588, 97589, 97590, 97591, 97592, 97593) c6( ) c7(bxC, B, aW, aT, dL, fr, ik, eL, iu); #c1(TAGLN2) c2(NP_003555) c3(transgelin-2 isoform b) c4( ) c5(139161, 139162, 139163, 139164, 139165, 139166, 139167, 139168, 139169, 139170, 139171, 139172, 139173) c6( ) c7(bxC, B, aW, aT, dL, fr, iK, eL, iu); #c1(TAL1) c2(NP_001277334) c3(T-cell acute lymphocytic leukemia protein 1 isoform 1) c4(109340, 109341) c5(109342, 109343, 109344, 109345, 109346, 109347, 109348, 109349, 109350, 109351, 109352, 109353, 109354) c6( ) c7(bdo, T, fM, dA, iW, DV, oA, jh, bcE, bS, gf, ct, ci, v, pz, hF, aQF, eJ, wD, x, PW, oT, fX, dE, qh, aql, oW, kH, aT, hn, dL, K, bXV, eL, iL, asp, je, aW, Y, ff, apK, en, Eg, Z, pm, pb, hq, ck, rR, iM, cKf, iT, au, pg, dW, zv, k, adg, y, bt); #c1(TAL1) c2(XP_011540349) c3(T-cell acute lymphocytic leukemia protein 1 isoform X2) c4( ) c5(338814, 338815, 338816, 338817) c6( ) c7(bdo, T, fM, dA, iW, DV, oA, jh, bcE, bS, gf, ct, ci, v, pz, hF, aQF, eJ, wD, x, PW, oT, EX, dE, qh, aql, oW, KH, aT, hn, dL, K, bXV, eL, iL, asp, je, aW, Y, ff, apK, en, Eg, Z, pm, pb, hq, ck, rR, iM, cKf, iT, au, pg, dW, zv, k, adg, y, bt); #c1(TAL2) c2(NP_005412) c3(T-cell acute lymphocytic leukemia protein 2) c4(155841) c5(155842, 155843, 155844, 155845, 155846, 155847, 155848) c6( ) c7(je, iM); #c1(TALDO1) c2(NP_006746) c3(transaldolase) c4(167646, 167647) c5(167648, 167649, 167650, 167651, 167652, 167653, 167654, 167655, 167656, 167657, 167658, 167659) c6( ) c7(gk, aBb, qb, yk, ff, al, ik, CT, kH, cKg, dq, lo, aj, hy, aBp, en, an, ars, V, B, J); #c1(TAMM41) c2(NP_620162) c3(phosphatidate cytidylyltransferase, mitochondrial isoform b precursor) c4(237430) c5(237431, 237432, 237433, 237434, 237435, 237436) c6( ) c7(od); #c1(TANC1) c2(NP_203752) c3(protein TANC1 isoform 1) c4( ) c5(228507, 228508, 228509, 228510, 228511) c6( ) c7(bv, j); #c1(TANC1) c2(XP_005246975) c3(protein TANC1 isoform X8) c4(267571, 267572) c5(267573) c6( ) c7(bv, j); #c1(TANC1) c2(XP_011510348) c3(protein TANC1 isoform X2) c4( ) c5(300899) c6( ) c7(bv, j); #c1(TANC1) c2(XP_011510360) c3(protein TANC1 isoform X14) c4(300900) c5(300901, 300902, 300903, 300904, 300905, 300906) c6( ) c7(bv, j); #c1(TANC2) c2(XP_005257260) c3(protein TANC2 isoform X1) c4(275014) c5(275015, 275016, 275017, 275018, 275019, 275020) c6( ) c7(aW, aT); #c1(TANC2) c2(XP_011522904) c3(protein TANC2 isoform X9) c4(317387) c5(317388, 317389, 317390, 317391, 317392, 317393) c6( ) c7(aW, aT); #c1(TANGO2) c2(NP_001270045) c3(transport and Golgi organization protein 2 homolog isoform a) c4(103362, 103363, 103364) c5(103365, 103366, 103367, 103368, 103369, 103370, 103371, 103372, 103373, 103374, 103375, 103376, 103377, 103378, 103379, 103380, 103381) c6( ) c7(ac); #c1(TANGO2) c2(NP_001270058) c3(transport and Golgi organization protein 2 homolog isoform b) c4(103382) c5(103383, 103384, 103385, 103386, 103387, 103388) c6( ) c7(ac); #c1(TANGO2) c2(NP_001270083) c3(transport and Golgi organization protein 2 homolog isoform c) c4( ) c5(103389, 103390, 103391, 103392, 103393, 103394) c6( ) c7(ac); #c1(TANGO2) c2(NP_001270115) c3(transport and Golgi organization protein 2 homolog isoform d) c4(103395) c5(103396, 103397, 103398, 103399, 103400, 103401, 103402, 103403, 103404, 103405, 103406, 103407, 103408, 103409) c6( ) c7(ac); #c1(TANGO2) c2(NP_001270128) c3(transport and Golgi organization protein 2 homolog isoform e) c4(103410) c5( ) c6( ) c7(ac); #c1(TANGO2) c2(NP_001270144) c3(transport and Golgi organization protein 2 homolog isoform f) c4(103411) c5(103412, 103413, 103414, 103415, 103416, 103417) c6( ) c7(ac); #c1(TANGO2) c2(NP_001270164) c3(transport and Golgi organization protein 2 homolog isoform g) c4(103418) c5(103419, 103420, 103421, 103422, 103423) c6( ) c7(ac); #c1(TANGO2) c2(NP_001270177) c3(transport and Golgi organization protein 2 homolog isoform h) c4( ) c5(103424, 103425, 103426, 103427, 103428, 103429, 103430) c6( ) c7(ac); #c1(TANK) c2(NP_004171) c3(TRAF family member-associated NF-kappa-B activator isoform a) c4( ) c5(144051, 144052, 144053, 144054, 144055, 144056, 144057, 144058) c6( ) c7(bM, T, aC, fR, jx, jh, fd, bB, j, qj, eA, aE, aT, h, K, Od, cp, eG, d, aW, Y, eZ, as, bz, yE, bE, cj, y, bt, iu, pC); #c1(TANK) c2(NP_597841) c3(TRAF family member-associated NF-kappa-B activator isoform b) c4(235209) c5(235210, 235211, 235212, 235213, 235214, 235215, 235216, 235217, 235218, 235219, 235220, 235221, 235222) c6( ) c7(bM, T, aC, fR, jx, jh, fd, bB, j, qj, eA, aE, aT, h, K, Od, cp, eG, d, aW, Y, eZ, as, bz, yE, bE, cj, y, bt, iu, pC); #c1(TANK) c2(XP_005246264) c3(TRAF family member-associated NF-kappa-B activator isoform X1) c4(267001, 267002) c5(267003, 267004, 267005, 267006, 267007) c6( ) c7(bM, T, aC, fR, jx, jh, fd, bB, j, qj, eA, aE, aT, h, K, Od, cp, eG, d, aW, Y, eZ, as, bz, yE, bE, cj, y, bt, iu, pC); #c1(TAOK1) c2(NP_079418) c3(serine/threonine-protein kinase TAO1 isoform 2) c4(218042) c5(218043, 218044, 218045, 218046, 218047, 218048, 218049) c6( ) c7(g, T, q, afW); #c1(TAOK1) c2(XP_011523362) c3(serine/threonine-protein kinase TAO1 isoform X1) c4(317884, 317885) c5(317886, 317887, 317888, 317889, 317890, 317891, 317892) c6( ) c7(g, T, q, afW); #c1(TAOK2) c2(NP_001238972) c3(serine/threonine-protein kinase TAO2 isoform 3) c4(88107) c5(88108, 88109, 88110, 88111, 88112, 88113, 88114) c6( ) c7(iu, T, ft, cbH, AQ, UF, aA, aF, K, g, Uw, hF, bt, af, bN, L); #c1(TAOK2) c2(NP_004774) c3(serine/threonine-protein kinase TAO2 isoform 2) c4(149511) c5(149512, 149513, 149514, 149515, 149516, 149517, 149518) c6( ) c7(iu, T, ft, cbH, AQ, UF, aA, aF, K, g, Uw, hF, bt, af, bN, L); #c1(TAOK2) c2(NP_057235) c3(serine/threonine-protein kinase TAO2 isoform 1) c4(189634) c5(189635, 189636, 189637, 189638, 189639, 189640) c6( ) c7(iu, T, ft, cbH, AQ, UF, aA, aF, K, g, Uw, hF, bt, af, bN, L); #c1(TADK2) c2(XP_011544290) c3(serine/threonine-protein kinase TAO2 isoform X7) c4(343439) c5(343440, 343441, 343442, 343443, 343444, 343445) c6( ) c7(iu, T, ft, cbH, AQ, UF, aA, aF, K, g, Uw, hF, bt, af, bN, L); #c1(TAOK3) c2(NP_057365) c3(serine/threonine-protein kinase TAO3) c4(190360, 190361, 190362, 190363) c5(190364, 190365, 190366, 190367, 190368, 190369, 190370, 190371, 190372, 190373, 190374, 190375, 190376, 190377, 190378, 190379) c6( ) c7(aW, ae, aT); #c1(TADK3) c2(XP_006719509) c3(serine/threonine-protein kinase TAO3 isoform X3) c4(292597, 292598, 292599, 292600, 292601) c5(292602, 292603, 292604, 292605, 292606, 292607, 292608, 292609, 292610, 292611, 292612, 292613, 292614, 292615, 292616, 292617, 292618, 292619, 292620, 292621, 292622, 292623, 292624, 292625, 292626) c6( ) c7(aW, ae, aT); #c1(TAOK3) c2(XP_011536740) c3(serine/threonine-protein kinase TAO3 isoform X2) c4( ) c5(334397, 334398, 334399, 334400, 334401, 334402, 334403, 334404, 334405, 334406, 334407, 334408, 334409, 334410, 334411) c6( ) c7(aW, ae, aT); #c1(TAOK3) c2(XP_011536742) c3(serine/threonine-protein kinase TAO3 isoform X4) c4( ) c5(334412) c6( ) c7(aW, ae, aT); #c1(TAP1) c2(NP_000584) c3(antigen peptide transporter 1 isoform 1) c4(13164, 13165) c5(13166, 13167, 13168, 13169, 13170, 13171, 13172, 13173, 13174, 13175, 13176, 13177, 13178, 13179, 13180, 13181, 13182, 13183, 13184, 13185, 13186, 13187, 13188, 13189, 13190, 13191, 13192, 13193, 13194, 13195, 13196, 13197, 13198, 13199, 13200, 13201, 13202, 13203, 13204, 13205, 13206, 13207, 13208, 13209, 13210, 13211, 13212, 13213, 13214, 13215, 13216, 13217, 13218, 13219, 13220, 13221, 13222, 13223, 13224) c6( ) c7(bM, T, FR, if, al, eb, bB, cL, qj, ci, dV, ja, Ds, K, hH, akX, cp, Jl, aW, asD, hl, ml, eE, uC, mK, eM, Jr, aiM, cKh, fr, qN, ai, eW, ft, cU, h, ey, A, mR, sp, kp, CQ, Zj, bo, JM, Xa, aAl, fL, aCD, B, Dw, bvt, jx, cz, bJw, j, V, q, eA, aT, Y, afS, r, afq, lo, aDm, iK, pb, pZ, AH, ao, vi, sY, Ke, aDy, ha, iD, hX, F, hD, uQ, atJ, d, cr, eZ, aF, bz, bE, z, aCl, y, WX, X); #c1(TAP1) c2(NP_001278951) c3(antigen peptide transporter 1 isoform 2) c4(111087) c5( ) c6( ) c7(bM, T, FR, if, al, eb, bB, cL, qj, ci, dV, ja, Ds, K, hH, akX, cp, Jl, aW, asD, hl, ml, eE, uC, mK, eM, Jr, aiM, cKh, fr, qN, ai, eW, ft, cU, h, ey, A, mR, sp, kp, CQ, Zj, bo, JM, Xa, aAl, fL, aCD, B, Dw, bvt, jx, cz, bJw, j, V, q, eA, aT, Y, afS, r, afq, lo, aDm, iK, pb, pZ, AH, ao, vi, sY, Ke, aDy, ha, iD, hX, F, HD, uQ, atJ, d, cr, eZ, aF, bz, bE, z, aCl, y, WX, X); #c1(TAP2) c2(NP_000535) c3 (antigen peptide transporter 2 isoform 1) c4(12159) c5(12160, 12161, 12162, 12163, 12164, 12165, 12166, 12167, 12168, 12169, 12170, 12171, 12172, 12173, 12174, 12175, 12176, 12177) c6( ) c7(bM, T, os, Dw, fR, Ke, bvt, aDy, if, pf, jx, iW, fN, eb, n, cl, blw, oZ, fr, bX, qN, V, q, mC, ai, hX, eW, AW, F, od, hD, h, Ds, azm, bK, ey, hu, hH, af, uQ, hM, A, mR, Jl, axu, Y, asD, afS, hl, r, ml, cr, lo, aDm, hp, gu, eZ, afq, eE, uC, pb, Zj, y, pZ, AH, JM, cj, Da, Xa, FL, lf, WX, ao, vi); #c1(TAP2) c2(NP_001276972) c3(antigen peptide transporter 2 isoform 3) c4(108862, 108863) c5(108864, 108865, 108866, 108867, 108868, 108869, 108870, 108871, 108872, 108873, 108874, 108875, 108876, 108877, 108878, 108879, 108880, 108881, 108882) c6( ) c7(bM, T, os, Dw, FR, Ke, bvt, aDy, if, pf, jx, iW, fN, eb, n, cL, bJw, oZ, fr, bX, qN, V, q, mC, ai, hX, eW, AW, F, od, hD, h, Ds, azm, bK, ey, hu, hH, af, uQ, hM, A, mR, Jl, axu, Y, asD, afS, hl, r, m, cr, lo, aDm, hp, gu, eZ, afq, eE, uC, pb, Zj, y, pZ, AH, JM, cj, Da, Xa, fl, lf, WX, ao, vi); #c1(TAP2) c2(NP_061313) c3(antigen peptide transporter 2 isoform 2) c4(199940, 199941, 199942, 199943, 199944) c5(199945, 199946, 199947, 199948, 199949, 199950, 199951, 199952, 199953, 199954, 199955, 199956, 199957, 199958, 199959, 199960, 199961, 199962, 199963, 199964, 199965, 199966, 199967, 199968, 199969, 199970, 199971, 199972, 199973, 199974, 199975, 199976, 199977, 199978, 199979, 199980, 199981, 199982, 199983, 199984, 199985, 199986, 199987, 199988, 199989, 199990, 199991, 199992, 199993, 199994, 199995, 199996, 199997, 199998, 199999, 200000, 200001, 200002, 200003, 200004, 200005, 200006, 200007, 200008, 200009) c6( ) c7(bM, T, os, Dw, fR, Ke, bvt, aDy, if, pf, jx, iW, fN, eb, n, cl, bJw, oZ, fr, bX, qN, V, q, mC, ai, hX, eW, AW, F, od, hD, h, Ds, azm, bK, ey, hu, hH, af, uQ, hM, A, mR, Jl, axu, Y, asD, afS, hl, r, ml, cr, lo, aDm, hp, gu, eZ, afq, eE, uC, pb, Zj, y, pZ, AH, JM, cj, Da, Xa, FL, lf, WX, ao, vi); #c1(TAPBP) c2(NP_003181) c3(tapasin isoform 1 precursor) c4(135994, 135995, 135996, 135997, 135998, 135999) c5(136000, 136001, 136002, 136003, 136004, 136005, 136006, 136007, 136008, 136009, 136010, 136011, 136012, 136013, 136014, 136015, 136016, 136017, 136018, 136019, 136020, 136021, 136022, 136023, 136024, 136025, 136026, 136027, 136028, 136029, 136030, 136031, 136032, 136033, 136034, 136035, 136036, 136037, 136038, 136039, 136040, 136041, 136042, 136043, 136044, 136045, 136046, 136047, 136048, 136049, 136050, 136051, 136052, 136053, 136054, 136055, 136056, 136057, 136058, 136059, 136060, 136061, 136062, 136063, 136064, 136065, 136066) c6( ) c7(bM, aC, bxC, dA, bxK, bvt, aDy, if, jx, cL, cKi, ci, qN, fr, q, o, ft, h, A, d, mR, Y, eZ, r, lo, g, bz, Zj, eM, yW); #c1(TAPBP) c2(NP_757345) c3(tapasin isoform 2 precursor) c4(248633, 248634) c5(248635, 248636, 248637, 248638, 248639, 248640, 248641, 248642, 248643, 248644, 248645, 248646, 248647, 248648, 248649, 248650, 248651, 248652, 248653, 248654, 248655, 248656, 248657) c6( ) c7(bM, aC, bxC, dA, bxK, bvt, aDy, if, jx, cL, cKi, ci, qN, fr, q, o, ft, h, A, d, mR, Y, eZ, r, lo, g, bz, Zj, eM, yW); #c1(TAPBP) c2(NP_757346) c3(tapasin isoform 3 precursor) c4(248658, 248659, 248660, 248661) c5(248662, 248663, 248664, 248665, 248666, 248667, 248668, 248669, 248670, 248671, 248672, 248673, 248674, 248675, 248676, 248677, 248678, 248679, 248680, 248681, 248682, 248683, 248684, 248685, 248686, 248687, 248688, 248689, 248690, 248691, 248692, 248693, 248694, 248695, 248696, 248697, 248698, 248699, 248700, 248701, 248702) c6( ) c7(bM, aC, bxC, dA, bxK, bvt, aDy, if, jx, cL, cKi, ci, qN, fr, q, o, ft, h, A, d, mR, Y, eZ, r, lo, g, bz, Zj, eM, yW); #c1(TAPBP) c2(XP_011513130) c3(tapasin isoform X1) c4(304946) c5(304947, 304948, 304949, 304950, 304951, 304952, 304953, 304954, 304955, 304956, 304957, 304958, 304959, 304960) c6( ) c7(bM, aC, bxC, dA, bxK, bvt, aDy, if, jx, cL, cKi, ci, qN, fr, q, o, ft, h, A, d, mR, Y, eZ, r, lo, g, bz, Zj, eM, yW); #c1(TAPBPL) c2(NP_060479) c3(tapasin-related protein precursor) c4(196152) c5(196153, 196154, 196155, 196156, 196157, 196158) c6( ) c7(af); #c1(TARBP1) c2(NP_005637) c3(probable methyltransferase TARBP1) c4(158182) c5(158183, 158184, 158185, 158186, 158187, 158188, 158189, 158190) c6( ) c7(aw, j); #c1(TARBP2) c2(NP_599150) c3(RISC-loading complex subunit TARBP2 isoform a) c4(235631, 235632) c5(235633, 235634, 235635, 235636, 235637, 235638, 235639, 235640) c6( ) c7(es, T, id, yk, bfz, aT, iv, h, fr, ci, as, ne, ad); #c1(TARBP2) c2(XP_005269172) c3(RISC-loading complex subunit TARBP2 isoform X1) c4( ) c5(282420, 282421, 282422, 282423, 282424, 282425, 282426, 282427) c6( ) c7(es, T, id, yk, bfz, aT, iv, h, fr, ci, as, ne, ad); #c1(TARP) c2(XP_011513708) c3(TCR gamma alternate reading frame protein isoform X2) c4(305996) c5(305997, 305998, 305999, 306000, 306001, 306002) c6( ) c7(bM, aGC, ha, jh, akW, ad, x, fx, afU, KH, hn, bK, lQ, ae, xR, Ca, ck, ou, coi, cj, k, i, y, LS); #c1(TARS) c2(NP_001245366) c3(threonine-tRNA ligase, cytoplasmic isoform 1) c4(93273, 93274, 93275) c5(93276, 93277, 93278, 93279, 93280, 93281, 93282, 93283, 93284, 93285, 93286, 93287, 93288, 93289, 93290, 93291, 93292, 93293, 93294, 93295, 93296, 93297, 93298, 93299, 93300, 93301, 93302, 93303) c6( ) c7(ds); #c1(TARS) c2(NP_001245367) c3(threonine-tRNA ligase, cytoplasmic isoform 2) c4(93304, 93305) c5(93306, 93307, 93308, 93309, 93310, 93311) c6( ) c7(ds); #c1(TARSL2) c2(NP_689547) c3(probable threonine-tRNA ligase 2, cytoplasmic) c4(243062, 243063, 243064) c5(243065, 243066, 243067, 243068, 243069, 243070, 243071, 243072, 243073, 243074, 243075, 243076, 243077, 243078, 243079, 243080, 243081, 243082) c6( ) c7(ds); #c1(TAS2R1) c2(NP_062545) c3(taste receptor type 2 member 1) c4(201908) c5(201909, 201910, 201911, 201912, 201913, 201914) c6( ) c7(bM, fq, ac, bN); #c1(TAS2R10) c2(NP_076410) c3(taste receptor type 2 member 10) c4(214076) c5(214077, 214078, 214079, 214080, 214081, 214082) c6( ) c7(aS); #c1(TAS2R13) c2(NP_076409) c3(taste receptor type 2 member 13) c4(214069) c5(214070, 214071, 214072, 214073, 214074, 214075) c6( ) c7(bL, ae, aC, wE, sM, Gd, mf, dG, K, KM, ad); #c1(TAS2R14) c2(NP_076411) c3(taste receptor type 2 member 14) c4(214083) c5(214084, 214085, 214086, 214087, 214088, 214089, 214090) c6( ) c7(v, r); #c1(TAS2R16) c2(NP_058641) c3(taste receptor type 2 member 16) c4(192906) c5(192907, 192908, 192909, 192910, 192911, 192912) c6( ) c7(aNU, xY, Gs, XY); #c1(TAS2R38) c2(NP_789787) c3(taste receptor type 2 member 38) c4(252294) c5(252295, 252296, 252297, 252298, 252299, 252300) c6( ) c7(ka, T, aCh, AS, Dd, n, hz, bS, j, ct, ci, aon, v, xY, q, aCi, Si, Vp, hd, xy, azj, afm, nv, Qg, Db, aT, aNU, Fx, jf, aCg, aW, Gv, r, aFd, Z, zV, aAW, cKj, qJ, sS, nM, nB, hk, ki, Gs, YH, AN); #c1(TAS2R50) c2(NP_795371) c3(taste receptor type 2 member 50) c4(252437) c5(252438, 252439, 252440, 252441, 252442, 252443) c6( ) c7(ac, aw, e, gZ, j, eu); #c1(TAS2R60) c2(NP_803186) c3(taste receptor type 2 member 60) c4(252547) c5(252548, 252549, 252550, 252551, 252552, 252553) c6( ) c7(Y); #c1(TAS2R9) c2(NP_076406) c3(taste receptor type 2 member 9) c4(214062) c5(214063, 214064, 214065, 214066, 214067, 214068) c6( ) c7(GE); #c1(TASP1) c2(NP_060184) c3(threonine aspartase 1 precursor) c4(194643, 194644) c5(194645, 194646, 194647, 194648, 194649, 194650, 194651) c6( ) c7(B); #c1(TASP1) c2(XP_011527592) c3(threonine aspartase 1 isoform X2) c4(322461, 322462, 322463) c5(322464, 322465, 322466, 322467, 322468, 322469, 322470, 322471, 322472) c6( ) c7(B); #c1(TASP1) c2(XP_011527597) c3(threonine aspartase 1 isoform X5) c4( ) c5(322473, 322474, 322475, 322476, 322477, 322478, 322479, 322480, 322481, 322482, 322483, 322484, 322485, 322486, 322487, 322488, 322489, 322490, 322491) c6( ) c7(B); #c1(TAT) c2(NP_000344) c3(tyrosine aminotransferase) c4(7452) c5(7453, 7454, 7455, 7456, 7457, 7458, 7459, 7460, 7461, 7462, 7463, 7464, 7465, 7466, 7467, 7468, 7469, 7470, 7471, 7472, 7473, 7474, 7475, 7476, 7477, 7478, 7479, 7480, 7481, 7482, 7483, 7484, 7485, 7486, 7487, 7488, 7489, 7490, 7491, 7492, 7493, 7494, 7495, 7496, 7497, 7498, 7499, 7500, 7501, 7502, 7503, 7504, 7505, 7506, 7507, 7508, 7509, 7510, 7511, 7512, 7513, 7514, 7515, 7516, 7517, 7518, 7519, 7520, 7521, 7522, 7523, 7524, 7525, 7526, 7527, 7528, 7529, 7530, 7531, 7532, 7533, 7534, 7535, 7536, 7537, 7538, 7539, 7540, 7541, 7542, 7543, 7544, 7545, 7546, 7547, 7548, 7549, 7550, 7551, 7552, 7553, 7554, 7555, 7556, 7557, 7558, 7559, 7560, 7561, 7562, 7563, 7564, 7565, 7566, 7567, 7568, 7569, 7570, 7571, 7572, 7573, 7574, 7575, 7576, 7577, 7578, 7579, 7580, 7581, 7582, 7583, 7584, 7585, 7586, 7587, 7588, 7589, 7590, 7591, 7592, 7593, 7594, 7595, 7596, 7597) c6(7598) c7(bL, T, DM, cKk, fM, Ke, e, DV, jh, aiC, bS, fr, ct, C, aiA, nA, ai, ad, eA, eJ, ip, x, eW, aE, pX, aT, h, cy, K, ae, pi, mu, le, aiB, qt, aW, oV, CM, cr, yq, gu, as, Z, kk, cm, bz, ek, ck, pH, ok, il, c, eM, bE, Uq, U, QT, QF, aiD, mf, k, m, EY, be, ym, bt, kS, B, y); #c1(TATDN1) c2(NP_001139632) c3(putative deoxyribonuclease TATDN1 isoform b) c4( ) c5(59579, 59580, 59581, 59582, 59583, 59584, 59585, 59586, 59587, 59588) c6( ) c7(B); #c1(TATDN1) c2(NP_114415) c3(putative deoxyribonuclease TATDN1 isoform a) c4(222675, 222676) c5(222677, 222678, 222679, 222680, 222681, 222682, 222683, 222684) c6( ) c7(B); #c1(TATDN1) c2(XP_006716729) c3(putative deoxyribonuclease TATDN1 isoform X2) c4(290445) c5( ) c6( ) c7(B); #c1(TAX1BP1) c2(NP_001073333) c3(tax1-binding protein 1 isoform 2) c4(35885) c5(35886, 35887, 35888, 35889, 35890, 35891, 35892, 35893, 35894, 35895) c6( ) c7(y, hy); #c1(TAX1BP1) c2(NP_001193831) c3(tax1-binding protein 1 isoform 3) c4(82703) c5(82704, 82705, 82706, 82707, 82708, 82709) c6( ) c7(y, hy); #c1(TAX1BP1) c2(NP_006015) c3(tax1-binding protein 1 isoform 1) c4(161188) c5(161189, 161190, 161191, 161192, 161193, 161194) c6( ) c7(y, hy); #c1(TAX1BP1) c2(XP_005249957) c3(tax1-binding protein 1 isoform X1) c4(269944, 269945) c5(269946, 269947, 269948, 269949, 269950, 269951, 269952, 269953, 269954, 269955, 269956, 269957) c6( ) c7(y, hy); #c1(TAX1BP3) c2(NP_055419) c3(tax1-binding protein 3 isoform 1) c4(181036) c5(181037, 181038, 181039, 181040, 181041, 181042) c6( ) c7(d, eA); #c1(TAZ) c2(NP_000107) c3(tafazzin isoform 1) c4(1816, 1817) c5(1818, 1819, 1820, 1821, 1822, 1823, 1824, 1825, 1826, 1827, 1828, 1829) c6( ) c7(gV, T, atk, fd, bB, afK, bS, ct, aoM, q, cd, vN, eA, cc, aT, vc, aWs, hG, aW, Y, asy, cmH, aNq, r, bp, Z, kC, Qt, dr, bfW, Bt, Dy, eM, bE, mx, wB, dy, m, lM); #c1(TAZ) c2(NP_851828) c3(tafazzin isoform 2) c4(254659) c5(254660, 254661, 254662, 254663, 254664, 254665) c6( ) c7(gV, T, atk, fd, bB, afk, bS, ct, aoM, q, cd, vN, eA, cc, al, vc, aWs, hG, aW, Y, asy, cmH, aNq, r, bp, Z, kC, Qt, dr, bfW, Bt, Dy, eM, bE, mx, wB, dy, m, lM); #c1(TAZ) c2(NP_851829) c3(tafazzin isoform 3) c4(254666) c5(254667, 254668, 254669, 254670, 254671, 254672) c6( ) c7(gV, T, atk, fd, bB, afk, bS, ct, aoM, q, cd, vN, eA, cc, aT, vc, aWs, hG, aW, Y, asy, cmH, aNq, r, bp, Z, kC, Qt, dr, bfW, Bt, Dy, eM, bE, mx, wB, dy, m, lM); #c1(TAZ) c2(NP_851830) c3(tafazzin isoform 4) c4(254673) c5(254674, 254675, 254676, 254677, 254678, 254679) c6( ) c7(gV, T, atk, fd, bB, afK, bS, ct, aoM, q, cd, vN, eA, cc, aT, vc, aWs, hG, aW, Y, asy, cmH, aNq, r, bp, Z, KC, Qt, dr, bfW, Bt, Dy, eM, bE, mx, wB, dy, m, lM); #c1(TAZ) c2(XP_011529491) c3(tafazzin isoform X4) c4( ) c5(324956) c6( ) c7(gV, T, atk, fd, bB, afK, bS, ct, aoM, q, cd, vN, eA, cc, aT, vc, aWs, hG, aW, Y, asy, cmH, aNq, r, bp, Z, kC, Qt, dr, bfW, Bt, Dy, eM, bE, mx, wB, dy, m, lM); #c1(TBATA) c2(XP_011537769) c3(protein TBATA isoform X5) c4(335308) c5(335309) c6( ) c7(aW, lO, V, h, aT); #c1(TBATA) c2(XP_011537770) c3(protein TBATA isoform X6) c4( ) c5(335310, 335311, 335312, 335313, 335314) c6( ) c7(aW, lO, V, h, aT); #c1(TBATA) c2(XP_011537785) c3(protein TBATA isoform X18) c4(335315) c5( ) c6( ) c7(aW, lO, V, h, aT); #c1(TBATA) c2(XP_011537787) c3(protein TBATA isoform X21) c4( ) c5(335316, 335317, 335318, 335319, 335320, 335321) c6( ) c7(aW, lO, V, h, aT); #c1(TBC1D1) c2(NP_001240841) c3(TBC1 domain family member 1 isoform 2) c4(88937) c5(88938, 88939, 88940, 88941, 88942, 88943) c6( ) c7(ac, vr, v, bS, qM, bSo); #c1(TBC1D1) c2(NP_055988) c3(TBC1 domain family member 1 isoform 1) c4(184952) c5(184953, 184954, 184955, 184956, 184957, 184958, 184959) c6( ) c7(ac, vr, v, bS, qM, bSo); #c1(TBC1D1) c2(XP_011511963) c3(TBC1 domain family member 1 isoform X3) c4(302612) c5( ) c6( ) c7(ac, vr, v, bS, qM, bSo); #c1(TBC1D1) c2(XP_011511965) c3(TBC1 domain family member 1 isoform X7) c4( ) c5(302613) c6( ) c7(ac, vr, v, bS, qM, bSo); #c1(TBC1D1) c2(XP_011511966) c3(TBC1 domain family member 1 isoform X8) c4(302614) c5(302615, 302616, 302617, 302618, 302619, 302620) c6( ) c7(ac, vr, v, bS, qM, bSo); #c1(TBC1D1) c2(XP_011511967) c3(TBC1 domain family member 1 isoform X9) c4( ) c5(302621, 302622, 302623) c6( ) c7(ac, vr, v, bS, qM, bSo); #c1(TBC1D1) c2(XP_011511972) c3(TBC1 domain family member 1 isoform X16) c4( ) c5(302624, 302625, 302626, 302627, 302628) c6( ) c7(ac, vr, v, bS, qM, bSo); #c1(TBC1D15) c2(NP_001139685) c3(TBC1 domain family member 15 isoform 3) c4(59703, 59704) c5(59705, 59706, 59707, 59708, 59709, 59710, 59711, 59712) c6( ) c7(bk); #c1(TBC1D15) c2(NP_001139686) c3(TBC1 domain family member 15 isoform 2) c4(59713) c5(59714, 59715, 59716, 59717, 59718, 59719, 59720, 59721, 59722) c6( ) c7(bK); #c1(TBC1D15) c2(NP_073608) c3(TBC1 domain family member 15 isoform 1) c4(213058) c5(213059, 213060, 213061, 213062, 213063, 213064, 213065, 213066, 213067, 213068, 213069, 213070, 213071, 213072, 213073) c6( ) c7(bK); #c1(TBC1D16) c2(NP_001258774) c3(TBC1 domain family member 16 isoform c) c4( ) c5(96522, 96523, 96524, 96525, 96526, 96527) c6( ) c7(Y, ac); #c1(TBC1D16) c2(NP_061893) c3(TBC1 domain family member 16 isoform a) c4(201009, 201010) c5(201011, 201012, 201013, 201014, 201015, 201016) c6( ) c7(Y, ac); #c1(TBC1D2) c2(NP_001254500) c3(TBC1 domain family member 2A isoform 1) c4(94533, 94534, 94535) c5(94536, 94537, 94538, 94539, 94540, 94541, 94542, 94543, 94544, 94545, 94546, 94547) c6( ) c7(Na, V, Nc); #c1(TBC1D2) c2(NP_001254501) c3(TBC1 domain family member 24 isoform 3) c4( ) c5(94548, 94549, 94550, 94551, 94552, 94553) c6( ) c7(Na, V, Nc); #c1(TBC1D2) c2(NP_060891) c3(TBC1 domain family member 2A isoform 2) c4(198425) c5(198426, 198427, 198428, 198429, 198430, 198431) c6( ) c7(Na, V, Nc); #c1(TBC1D20) c2(NP_653229) c3(TBC1 domain family member 20) c4(239223, 239224) c5(239225, 239226, 239227, 239228, 239229, 239230, 239231, 239232, 239233, 239234, 239235, 239236, 239237, 239238, 239239, 239240) c6( ) c7(cxV, ac, gU, cKl); #c1(TBC1D22A) c2(NP_001271232) c3(TBC1 domain family member 22A isoform b) c4(103673) c5(103674, 103675, 103676, 103677, 103678, 103679, 103680, 103681, 103682, 103683, 103684, 103685) c6( ) c7(ac, aw, oe, an, fq, cd); #c1(TBC1D22A) c2(NP_055161) c3(TBC1 domain family member 22A isoform a) c4(179534, 179535, 179536) c5(179537, 179538, 179539, 179540, 179541, 179542, 179543, 179544, 179545, 179546, 179547, 179548) c6( ) c7(ac, aw, oe, an, fq, cd); #c1(TBC1D22A) c2(XP_005261553) c3(TBC1 domain family member 22A isoform X5) c4(277474) c5(277475, 277476, 277477, 277478, 277479, 277480) c6( ) c7(ac, aw, oe, an, fq, cd); #c1(TBC1D22B) c2(NP_060242) c3(TBC1 domain family member 22B) c4(194946) c5(194947, 194948, 194949, 194950, 194951, 194952) c6( ) c7(oe); #c1(TBC1D24) c2(NP_001186036) c3(TBC1 domain family member 24 isoform 1) c4(76916) c5(76917, 76918, 76919, 76920, 76921, 76922) c6( ) c7(cZ, beO, cko, ckm, ha, lr, cKn, aft, yZ, lm, nA, dd, yU, Bh, byk); #c1(TBC1D24) c2(NP_065756) c3(TBC1 domain family member 24 isoform 2) c4(205204) c5(205205, 205206, 205207, 205208, 205209, 205210, 205211, 205212, 205213, 205214, 205215) c6( ) c7(cZ, beO, cko, ckm, ha, lr, cKn, aft, yZ, lm, nA, dd, yU, Bh, byk); #c1(TBC1D25) c2(NP_002527) c3(TBC1 domain family member 25) c4(129563, 129564, 129565, 129566) c5(129567, 129568, 129569, 129570, 129571, 129572, 129573, 129574, 129575, 129576, 129577, 129578, 129579, 129580, 129581, 129582, 129583, 129584, 129585, 129586, 129587, 129588, 129589, 129590) c6( ) c7(bS, BG, nA, Vi, iJ, hq); #c1(TBC1D32) c2(NP_689943) c3(protein broad-minded) c4(244435) c5(244436, 244437, 244438, 244439, 244440, 244441) c6( ) c7(fq); #c1(TBC1D3C) c2(XP_006721970) c3(TBC1 domain family member 3C isoform X3) c4( ) c5(294506, 294507, 294508, 294509, 294510) c6( ) c7(ae, ad); #c1(TBC1D3F) c2(NP_115634) c3(TBC1 domain family member 3F) c4(223394, 223395, 223396) c5(223397, 223398, 223399, 223400, 223401, 223402, 223403, 223404, 223405, 223406, 223407, 223408, 223409, 223410, 223411) c6( ) c7(ad); #c1(TBC1D4) c2(NP_001273587) c3(TBC1 domain family member 4 isoform 2) c4(105365) c5(105366, 105367, 105368, 105369, 105370, 105371) c6( ) c7(bL, kl, oe, vr, jy, eE); #c1(TBC1D4) c2(NP_001273588) c3(TBC1 domain family member 4 isoform 3) c4(105372, 105373) c5(105374, 105375, 105376, 105377, 105378, 105379, 105380, 105381, 105382, 105383, 105384, 105385) c6( ) c7(bL, kl, oe, vr, jy, eE); #c1(TBC1D4) c2(NP_055647) c3(TBC1 domain family member 4 isoform 1) c4(182520) c5(182521, 182522, 182523, 182524, 182525, 182526, 182527) c6( ) c7(bL, kl, oe, vr, jy, eE); #c1(TBC1D4) c2(XP_005266662) c3(TBC1 domain family member 4 isoform X4) c4( ) c5(280273, 280274, 280275, 280276, 280277, 280278, 280279) c6( ) c7(bL, kl, oe, vr, jy, eE); #c1(TBC1D4) c2(XP_006719966) c3(TBC1 domain family member 4 isoform X3) c4(293116) c5( ) c6( ) c7(bL, kl, oe, vr, jy, eE); #c1(TBC1D5) c2(NP_001127853) c3(TBC1 domain family member 5 isoform a) c4(49388, 49389, 49390) c5(49391, 49392, 49393, 49394, 49395, 49396) c6( ) c7(T, cd); #c1(TBC1D5) c2(NP_055559) c3(TBC1 domain family member 5 isoform b) c4(181931) c5(181932, 181933, 181934, 181935, 181936, 181937, 181938, 181939, 181940, 181941, 181942, 181943, 181944, 181945, 181946, 181947, 181948, 181949, 181950, 181951, 181952, 181953) c6( ) c7(T, cd); #c1(TBC1D7) c2(NP_001137438) c3(TBC1 domain family member 7 isoform b) c4(55712, 55713, 55714, 55715, 55716) c5(55717, 55718, 55719, 55720, 55721, 55722, 55723, 55724, 55725, 55726, 55727, 55728, 55729, 55730, 55731, 55732, 55733, 55734, 55735, 55736, 55737, 55738, 55739, 55740, 55741, 55742, 55743, 55744, 55745, 55746, 55747, 55748, 55749) c6( ) c7(hl, eM, e, hp, PY, nA, bz); #c1(TBC1D7) c2(NP_001245386) c3(TBC1 domain family member 7 isoform c) c4(93421) c5(93422, 93423, 93424, 93425, 93426, 93427) c6( ) c7(hl, eM, e, hp, PY, nA, bz); #c1(TBC1D7) c2(NP_057579) c3(TBC1 domain family member 7 isoform a) c4(191423, 191424, 191425, 191426, 191427, 191428) c5(191429, 191430, 191431, 191432, 191433, 191434, 191435, 191436, 191437, 191438, 191439, 191440, 191441, 191442, 191443, 191444, 191445, 191446, 191447, 191448, 191449, 191450, 191451, 191452, 191453, 191454, 191455, 191456, 191457, 191458, 191459, 191460, 191461, 191462, 191463, 191464, 191465, 191466, 191467, 191468, 191469) c6( ) c7(hl, eM, e, hp, PY, nA, bz); #c1(TBC1D8) c2(NP_001095896) c3(TBC1 domain family member 8) c4(40926) c5(40927, 40928, 40929, 40930, 40931, 40932) c6( ) c7(T, dB, ds, h, fr, m, ir, t, bA); #c1(TBC1D8) c2(XP_005263920) c3(TBC1 domain family member 8 isoform X2) c4(278576) c5(278577, 278578, 278579, 278580, 278581, 278582) c6( ) c7(T, dB, ds, h, fr, m, ir, t, bA); #c1(TBC1D9) c2(NP_055945) c3(TBC1 domain family member 9) c4(184595) c5(184596, 184597, 184598, 184599, 184600, 184601) c6( ) c7(T, hV, if, al, bO, fd, bB, gA, ci, ip, x, bv, aE, gH, iX, M, dV, iv, gs, K, hu, eL, bc, eG, iE, aW, az, ih, hN, aB, g, ir, eF, cm, bw, iP, eM, iA, iT, cj, iS, k, m, eg, gn, in, ik, gD, fW, gp, aw, ht, n, fr, ct, eW, br, ft, ec, h, hn, dL, hB, is, dx, ia, hT, gi, iw, hQ, fe, aq, gG, iO, dB, hL, hj, hs, it, B, fM, dA, fZ, aA, hz, j, il, q, er, eA, hW, aT, dz, gz, jj, an, gS, ae, jf, gc, hM, iF, gP, iU, Y, ev, r, en, hf, cl, gl, t, ho, ik, hY, ck, iM, ac, hA, au, gX, hO, dy, ij, im, bt, ao, gx, ie, id, aD, ha, gN, bs, jh, bS, ah, hF, ad, hX, av, fX, ib, io, hP, ji, il, iz, gj, d, je, hG, cH, et, ff, gK, eH, eZ, gE, hp, aF, jg, iV, Z, iR, bz, bF, es, gk, bE, eB, y, ix); #c1(TBCA) c2(NP_001284667) c3(tubulin-specific chaperone A isoform 1) c4(112288) c5(112289, 112290, 112291, 112292, 112293, 112294) c6( ) c7(Ol, OV, zH, xZ, hE, zD, vc, v, m, eE, t); #c1(TBCA) c2(NP_004598) c3(tubulin-specific chaperone A isoform 2) c4(148028) c5(148029, 148030, 148031, 148032, 148033, 148034) c6( ) c7(Ol, OV, zH, xZ, hE, zD, vc, v, m, eE, t); #c1(TBCC) c2(NP_003183) c3 (tubulin-specific chaperone C) c4(136067) c5(136068, 136069, 136070, 136071, 136072, 136073, 136074, 136075, 136076, 136077) c6( ) c7(ck, T, iU, aW, bK, bt); #c1(TBCD) c2(NP_005984) c3(tubulin-specific chaperone D) c4(160978, 160979, 160980) c5(160981, 160982, 160983, 160984, 160985, 160986, 160987, 160988, 160989, 160990, 160991, 160992, 160993, 160994) c6( ) c7(dy, ac); #c1(TBCD) c2(XP_005256453) c3(tubulin-specific chaperone D isoform X1) c4(274184, 274185) c5(274186) c6( ) c7(dy, ac); #c1(TBCD) c2(XP_011521898) c3(tubulin-specific chaperone D isoform X12) c4(316505) c5(316506, 316507, 316508, 316509, 316510, 316511, 316512, 316513, 316514, 316515, 316516, 316517, 316518, 316519, 316520, 316521) c6( ) c7(dy, ac); #c1(TBCD) c2(XP_011521902) c3(tubulin-specific chaperone D isoform X16) c4( ) c5(316522, 316523, 316524, 316525, 316526) c6( ) c7(dy, ac); #c1(TBCE) c2(NP_001072983) c3(tubulin-specific chaperone E isoform a) c4(35435) c5(35436, 35437, 35438, 35439, 35440, 35441, 35442, 35443, 35444, 35445) c6( ) c7(oO, cKq, OI, OX, cKp, Az, Hz, h, v, K, OM, cKr, eW); #c1(TBCE) c2(NP_001274730) c3(tubulin-specific chaperone E isoform b) c4(106713) c5(106714, 106715, 106716, 106717, 106718, 106719) c6( ) c7(oO, cKq, OI, OX, cKp, Az, Hz, h, v, K, OM, cKr, eW); #c1(TBCEL) c2(NP_001123519) c3(tubulin-specific chaperone cofactor E-like protein) c4(47301) c5(47302, 47303, 47304, 47305, 47306, 47307, 47308, 47309, 47310, 47311) c6( ) c7(ao, g, K, rV); #c1(TBK1) c2(XP_005268867) c3(serine/threonine-protein kinase TBK1 isoform X1) c4(282139, 282140, 282141) c5(282142, 282143, 282144, 282145, 282146, 282147, 282148, 282149, 282150, 282151, 282152, 282153, 282154, 282155, 282156, 282157, 282158, 282159) c6( ) c7(oO, HQ, cZ, T, fR, jx, j, eh, eW, ad, hX, F, aT, K, dd, ae, afu, dH, aW, Y, eZ, lo, alb, CQ, dw, bz, pZ, eM, eg, hh, B); #c1(TBKBP1) c2(NP_055541) c3(TANK-binding kinase 1-binding protein 1) c4(181833, 181834) c5(181835, 181836, 181837, 181838, 181839, 181840, 181841, 181842, 181843, 181844, 181845, 181846) c6( ) c7(mR, fR); #c1(TBL1X) c2(NP_001132938) c3(F-box-like/WD repeat-containing protein TBL1X isoform a) c4(52688, 52689, 52690, 52691) c5(52692, 52693, 52694, 52695, 52696, 52697, 52698, 52699, 52700, 52701, 52702, 52703, 52704, 52705, 52706, 52707, 52708, 52709, 52710, 52711, 52712, 52713, 52714, 52715, 52716, 52717, 52718, 52719) c6( ) c7(VX, aW, mG, fc, al, im, ei, cP); #c1(TBL1X) c2(XP_011543873) c3 (F-box-like/WD repeat-containing protein TBL1X isoform X1) c4(343033) c5(343034, 343035, 343036, 343037, 343038, 343039, 343040, 343041, 343042, 343043, 343044, 343045, 343046, 343047, 343048, 343049) c6( ) c7(VX, aW, mG, fc, aT, im, ei, cP); #c1(TBL1XR1) c2(XP_005247828) c3(F-box-like/WD repeat-containing protein TBL1XR1 isoform X3) c4( ) c5(268001) c6( ) c7(x, ae, gj, T, Ew, if, fc, jh, n, hn, v, k, cP, ei, qN, ey, bz, ad); #c1(TBL1XR1) c2(XP_005247833) c3(F-box-like/WD repeat-containing protein TBL1XR1 isoform X4) c4( ) c5(268002, 268003, 268004, 268005, 268006, 268007) c6( ) c7(x, ae, gj, T, Ew, if, fc, jh, n, hn, v, k, cP, ei, qN, ey, bz, ad); #c1(TBL1XR1) c2(XP_011511445) c3(F-box-like/WD repeat-containing protein TBL1XR1 isoform X1) c4(302079, 302080, 302081, 302082, 302083, 302084, 302085, 302086, 302087, 302088) c5(302089, 302090, 302091, 302092, 302093, 302094, 302095, 302096, 302097, 302098, 302099, 302100, 302101, 302102, 302103, 302104, 302105, 302106, 302107, 302108, 302109, 302110, 302111, 302112, 302113, 302114, 302115, 302116, 302117, 302118, 302119, 302120, 302121, 302122, 302123, 302124, 302125, 302126, 302127, 302128, 302129, 302130, 302131, 302132, 302133, 302134, 302135, 302136, 302137, 302138, 302139, 302140, 302141, 302142, 302143, 302144, 302145, 302146, 302147, 302148, 302149) c6( ) c7(x, ae, gj, T, Ew, if, fc, jh, n, hn, v, k, cP, ei, qN, ey, bz, ad); #c1(TBL1Y) c2(NP_599021) c3(F-box-like/WD repeat-containing protein TBL1Y) c4(235542, 235543) c5(235544, 235545, 235546, 235547, 235548, 235549, 235550, 235551, 235552, 235553, 235554, 235555, 235556, 235557, 235558, 235559, 235560, 235561, 235562, 235563, 235564, 235565, 235566, 235567, 235568) c6( ) c7(aW, mG, fc, aT, im, j, cP, ei, bN); #c1(TBL1Y) c2(XP_005262629) c3(F-box-like/WD repeat-containing protein TBL1Y isoform X1) c4(278027) c5( ) c6( ) c7(aW, mG, fc, aT, im, j, cP, ei, bN); #c1(TBL2) c2(NP_036585) c3 (transducin beta-like protein 2 precursor) c4(174543, 174544) c5(174545, 174546, 174547, 174548, 174549, 174550, 174551, 174552, 174553, 174554, 174555, 174556) c6( ) c7(cO, bL); #c1(TBL3) c2(NP_006444) c3 (transducin beta-like protein 3) c4(165267) c5(165268, 165269, 165270, 165271, 165272, 165273, 165274, 165275, 165276, 165277) c6( ) c7(dy, ac); #c1(TBP) c2(NP_001165556) c3(TATA-box-binding protein isoform 2) c4(67757) c5(67758, 67759, 67760, 67761, 67762, 67763, 67764, 67765, 67766, 67767, 67768, 67769, 67770, 67771, 67772, 67773, 67774) c6( ) c7(T, cvj, fG, yY, rf, GC, qj, ci, jX, aGK, nA, q, aR, PW, akN, bgk, KB, aT, Wk, aU, yZ, dL, K, Wm, an, on, nJ, af, eL, A, s, aW, Fm, Y, r, fe, aq, EH, ckd, Rl, g, ajG, pb, GB, rR, VJ, ky, cEz, KB, Kv, bE, eD, U, lC, B, pC); #c1(TBP) c2(NP_003185) c3(TATA-box-binding protein isoform 1) c4(136078, 136079, 136080, 136081, 136082) c5(136083, 136084, 136085, 136086, 136087, 136088, 136089, 136090, 136091, 136092, 136093, 136094, 136095, 136096, 136097, 136098, 136099, 136100, 136101, 136102, 136103, 136104, 136105, 136106) c6( ) c7(T, cvj, fG, yY, rf, GC, qj, ci, jX, aGK, nA, q, aR, PW, akN, bgk, KB, aT, Wk, aU, yZ, dL, K, Wm, an, on, nJ, af, eL, A, s, aW, Fm, Y, r, fe, aq, EH, ckd, Rl, g, ajG, pb, GB, rR, VJ, ky, cEz, kB, Kv, bE, eD, U, lC, B, pC); #c1(TBPL1) c2(NP_004856) c3(TATA box-binding protein-like protein 1) c4(150335, 150336, 150337, 150338, 150339) c5(150340, 150341, 150342, 150343, 150344, 150345, 150346, 150347, 150348, 150349, 150350, 150351, 150352, 150353, 150354, 150355, 150356, 150357, 150358, 150359, 150360, 150361, 150362, 150363, 150364, 150365, 150366, 150367, 150368, 150369, 150370) c6( ) c7(bM, T, os, gU, yk, e, cV, cbc, aA, tc, bB, aNG, uu, ci, oZ, lN, fd, hF, q, cd, ad, auf, MR, KH, aT, vw, aYf, dL, vH, K, h, ae, eL, ajU, pl, je, ND, aW, Y, Gv, ff, hL, r, fe, aF, jh, en, iV, pl, vo, cm, dr, iP, ck, iM, eM, KB, lI, hk, bE, wB, U, nw, dy, WL, KS, B); #c1(TBPL2) c2(NP_950248) c3(TATA box-binding protein-like protein 2) c4(260931) c5(260932, 260933, 260934, 260935, 260936, 260937) c6( ) c7(dP, T, dD, iM, je, op, ea, vA, bo, kH, bB, dq, aj, eq, K, J); #c1(TBR1) c2(NP_006584) c3(T-box brain protein 1) c4(166396, 166397, 166398) c5(166399, 166400, 166401, 166402, 166403, 166404, 166405, 166406, 166407, 166408, 166409, 166410, 166411, 166412, 166413, 166414, 166415, 166416, 166417) c6( ) c7(nA, r); #c1(TBRG1) c2(NP_116200) c3(transforming growth factor beta regulator 1) c4(225716, 225717) c5(225718, 225719, 225720, 225721, 225722, 225723) c6( ) c7(ou); #c1(TBRG1) c2(XP_005271766) c3(transforming growth factor beta regulator 1 isoform X1) c4( ) c5(283572, 283573, 283574, 283575, 283576, 283577) c6( ) c7(ou); #c1(TBX1) c2(NP_005983) c3(T-box transcription factor TBX1 isoform B) c4(160971) c5(160972, 160973, 160974, 160975, 160976, 160977) c6( ) c7(bM, T, aiH, e, aiC, ci, Na, ahd, aW, Az, g, cAS, sv, pw, axN, ik, Zo, RX, n, aln, nA, bVE, md, aUb, OM, he, Gc, OX, aMH, aMJ, cKu, hq, aLi, cA, bD, iH, v, Eh, q, Mg, qK, yg, aT, bK, rz, qk, kl, Y, cKp, r, en, cl, bN, cKs, cKt, HK, FI, atk, acl, oA, vG, hF, agY, xt, hX, av, agd, Db, aQj, Pc, WZ, ff, aJk, gl, hp, S, jb, bz, dr, qa, Ew, aBU, bYB, auH, be, y); #c1(TBX1) c2(NP_542377) c3(T-box transcription factor TBX1 isoform A) c4(232289) c5(232290, 232291, 232292, 232293, 232294, 232295) c6( ) c7(bM, T, aiH, e, aiC, ci, Na, ahd, aW, Az, g, cAS, sv, pw, axN, ik, Zo, RX, n, aln, nA, bVE, md, aUb, OM, he, Gc, OX, aMH, aMJ, cKu, hq, aLi, cA, bD, iH, v, Eh, q, Mg, qK, yg, aT, bK, rz, qk, kl, Y, cKp, r, en, cl, bN, cKs, cKt, HK, Fl, atk, acl, oA, vG, hF, agY, xt, hX, av, agd, Db, aQj, Pc, WZ, ff, aJk, gl, hp, S, jb, bz, dr, qa, Ew, aBU, bYB, auH, be, y); #c1(TBX1) c2(NP_542378) c3(T-box transcription factor TBX1 isoform C) c4(232296, 232297) c5(232298, 232299, 232300, 232301, 232302, 232303, 232304, 232305, 232306, 232307, 232308) c6( ) c7(bM, T, aiH, e, aiC, ci, Na, ahd, aW, Az, g, cAS, sv, pw, axN, ik, Zo, RX, n, aln, nA, bVE, md, aUb, OM, he, Gc, OX, aMH, aMJ, cKu, hq, aLi, cA, bD, iH, v, Eh, q, Mg, qK, yg, aT, bK, rz, qk, kl, Y, cKp, r, en, cl, bN, cKs, cKt, HK, Fl, atk, acl, oA, vG, hF, agY, xt, hX, av, agd, Db, aQj, Pc, WZ, ff, aJk, gl, hp, S, jb, bz, dr, qa, Ew, aBU, bYB, auH, be, y); #c1(TBX10) c2(NP_005986) c3(T-box transcription factor TBX10) c4(160995) c5(160996, 160997, 160998, 160999, 161000, 161001) c6( ) c7(Na, Nc, apM, aOX); #c1(TBX10) c2(XP_011543294) c3(T-box transcription factor TBX10 isoform X1) c4( ) c5(342086) c6( ) c7(Na, Nc, apM, aOX); #c1(TBX18) c2(NP_001073977) c3(T-box transcription factor TBX18) c4(36755) c5(36756, 36757, 36758, 36759, 36760, 36761) c6( ) c7(eF, eH, j, Kw); #c1(TBX18) c2(XP_006715665) c3(T-box transcription factor TBX18 isoform X4) c4( ) c5(289701) c6( ) c7(eF, eH, j, Kw); #c1(TBX18) c2(XP_011534549) c3(T-box transcription factor TBX18 isoform X1) c4(331085) c5(331086, 331087, 331088, 331089, 331090, 331091) c6( ) c7(eF, eH, j, Kw); #c1(TBX19) c2(NP_005140) c3(T-box transcription factor TBX19) c4(153337, 153338, 153339, 153340) c5(153341, 153342, 153343, 153344, 153345, 153346, 153347, 153348, 153349, 153350, 153351, 153352, 153353, 153354, 153355, 153356, 153357, 153358, 153359, 153360, 153361, 153362, 153363, 153364, 153365) c6( ) c7(s, Wt, cz, aVh, vg, bS, OD, ml, aVL, bmd, bA); #c1(TBX2) c2(XP_011523460) c3(T-box transcription factor TBX2 isoform X1) c4(317983, 317984) c5(317985, 317986, 317987, 317988, 317989, 317990, 317991, 317992, 317993, 317994, 317995, 317996, 317997) c6( ) c7(md, T, aE, aW, Y, acl, aT, iv, dL, dy, ci, K, j, BM, el); #c1(TBX20) c2(NP_001071121) c3(T-box transcription factor TBX20 isoform 1) c4(35294) c5(35295, 35296, 35297, 35298, 35299, 35300) c6( ) c7(Mg, bwO, dD, cc, ac, rz, ea, acl, aw, al, gZ, bD, aaw, j, eq, cKv, hH, nC); #c1(TBX20) c2(NP_001159692) c3(T-box transcription factor TBX20 isoform 2) c4( ) c5(64600) c6( ) c7(Mg, bwO, dD, cc, ac, rz, ea, acl, aw, al, gZ, bD, aaw, j, eq, cKv, hH, nC); #c1(TBX21) c2(NP_037483) c3(T-box transcription factor TBX21) c4(175756) c5(175757, 175758, 175759, 175760, 175761, 175762, 175763) c6( ) c7(bM, Dw, FR, eX, bO, aA, xM, hz, lR, bh, pM, V, qW, eW, amW, zn, hX, b, cKw, hD, eV, Ds, A, cwV, aCx, eZ, BG, Z, eE, pb, ck, eU, dW, Tu, i, hE, y); #c1(TBX22) c2(NP_001103348) c3(T-box transcription factor TBX22 isoform 1) c4(41874) c5(41875, 41876, 41877, 41878, 41879, 41880, 41881, 41882, 41883, 41884, 41885, 41886) c6( ) c7(kn, Na, aVh, h, Nc, aml, aVg, bYQ, cKx, ao); #c1(TBX22) c2(NP_001103349) c3(T-box transcription factor TBX22 isoform 2) c4( ) c5(41887, 41888, 41889, 41890, 41891, 41892, 41893, 41894, 41895) c6( ) c7(kn, Na, aVh, h, Nc, aml, aVg, bYQ, cKx, ao); #c1(TBX22) c2(XP_006725480) c3(T-box transcription factor TBX22 isoform X1) c4(296823) c5( ) c6( ) c7(kn, Na, aVh, h, Nc, aml, aVg, bYQ, cKx, ao); #c1(TBX3) c2(NP_005987) c3(T-box transcription factor TBX3 isoform 1) c4(161002) c5(161003, 161004, 161005, 161006, 161007, 161008, 161009) c6( ) c7(T, acl, oe, aw, EQ, aA, cz, hz, nA, Dr, aT, dl, eL, af, aW, Y, r, aJk, aq, iR, t, ik, atb, oU, iO, ac, bE, dy, y, B); #c1(TBX3) c2(NP_057653) c3(T-box transcription factor TBX3 isoform 2) c4(192070, 192071) c5(192072, 192073, 192074, 192075, 192076, 192077, 192078, 192079, 192080, 192081, 192082, 192083) c6( ) c7(T, acl, oe, aw, EQ, aA, cz, hz, nA, Dr, aT, dL, eL, af, aW, Y, r, aJk, aq, iR, t, iK, atb, oU, iO, ac, bE, dy, y, B); #c1(TBX4) c2(NP_060958) c3(T-box transcription factor TBX4) c4(198887) c5( ) c6( ) c7(ID, ly, XW, bhl, aT, gl, cKy, aOm, lx); #c1(TBX4) c2(XP_011523792) c3(T-box transcription factor TBX4 isoform X1) c4( ) c5(318285) c6( ) c7(ID, ly, XW, bhl, aT, gl, cKy, aOm, lx); #c1(TBX4) c2(XP_011523793) c3(T-box transcription factor TBX4 isoform X2) c4( ) c5(318286, 318287, 318288, 318289, 318290, 318291) c6( ) c7(ID, ly, XW, bhl, aT, gl, cKy, aOm, lx); #c1(TBX4) c2(XP_011523796) c3(T-box transcription factor TBX4 isoform X3) c4(318292) c5(318293, 318294, 318295, 318296, 318297) c6( ) c7(ID, ly, XW, bhl, aT, gl, cKy, aOm, lx); #c1(TBX5) c2(NP_542448) c3(T-box transcription factor TBX5 isoform 3) c4( ) c5(232980, 232981, 232982, 232983, 232984, 232985) c6( ) c7(T, mr, atk, bkF, acl, aw, wL, EQ, cz, Hn, j, ci, BM, cd, afb, qH, md, cF, ft, aE, h, rz, eL, Gc, eG, are, eH, aF, eF, cm, atb, arB, gY, bhl, gZ, bD, vt, B); #c1(TBX5) c2(NP_852259) c3(T-box transcription factor TBX5 isoform 1) c4(254931, 254932, 254933, 254934) c5(254935, 254936, 254937, 254938, 254939, 254940, 254941, 254942, 254943, 254944, 254945, 254946, 254947, 254948, 254949, 254950, 254951, 254952, 254953, 254954, 254955, 254956, 254957) c6( ) c7(T, mr, atk, bkF, acl, aw, wL, EQ, cz, Hn, j, ci, BM, cd, afb, qH, md, cF, ft, aE, h, rz, eL, Gc, eG, are, eH, aF, eF, cm, atb, arB, gY, bhl, gZ, bD, vt, B); #c1(TBX6) c2(NP_004599) c3(T-box transcription factor TBX6) c4(148035, 148036) c5(148037, 148038, 148039, 148040, 148041, 148042, 148043, 148044, 148045, 148046, 148047, 148048, 148049, 148050, 148051, 148052, 148053) c6( ) c7(qb, lq, bZl, bQ, bxG, bBk, bfU); #c1(TBXA2R) c2(NP_963998) c3(thromboxane A2 receptor isoform beta) c4(262611) c5(262612, 262613, 262614, 262615, 262616, 262617) c6( ) c7(bM, T, cKz, xu, ea, aw, j, pM, q, ad, brX, jo, af, il, sp, r, as, eE, bz, ek, ac, z, JM, qq, dX, wx, Tu, m); #c1(TBXA2R) c2(XP_011526516) c3(thromboxane A2 receptor isoform X1) c4(320813) c5(320814, 320815, 320816, 320817, 320818, 320819) c6( ) c7(bM, T, cz, xu, ea, aw, j, pM, q, ad, brX, jo, af, iU, sp, r, as, eE, bz, ek, ac, z, JM, qq, dx, wx, Tu, m); #c1(TBXAS1) c2(NP_001052) c3(thromboxane-A synthase isoform 1) c4(34392, 34393, 34394) c5(34395, 34396, 34397, 34398, 34399, 34400, 34401, 34402, 34403, 34404, 34405, 34406, 34407, 34408, 34409, 34410, 34411, 34412, 34413, 34414, 34415) c6( ) c7(bM, sY, cKA, ea, dk, aw, uO, cz, ad, av, cF, fS, aT, lG, dl, jo, af, eu, aW, EU, uC, ek, tk, ac, wg, hc, hR, wx, m); #c1(TBXAS1) c2(NP_001159725) c3(thromboxane-A synthase isoform 3) c4(64630, 64631) c5(64632, 64633, 64634, 64635, 64636, 64637) c6( ) c7(bM, sY, cKA, ea, dk, aw, uO, cz, ad, av, cF, fS, aT, lG, dl, jo, af, eu, aW, EU, uC, ek, tk, ac, wg, hc, hR, wx, m); #c1(TBXAS1) c2(NP_001159726) c3(thromboxane-A synthase isoform 4) c4( ) c5(64638, 64639, 64640, 64641, 64642) c6( ) c7(bM, sY, cKA, ea, dk, aw, uO, cz, ad, av, cF, fS, aT, lG, dl, jo, af, eu, aW, EU, uC, ek, tk, ac, wg, hc, hR, wx, m); #c1(TBXAS1) c2(NP_112246) c3(thromboxane-A synthase isoform 2) c4(221101, 221102, 221103) c5(221104, 221105, 221106, 221107, 221108, 221109, 221110, 221111, 221112, 221113, 221114, 221115, 221116, 221117, 221118, 221119, 221120, 221121, 221122, 221123, 221124, 221125, 221126, 221127) c6( ) c7(bM, sY, cKA, ea, dk, aw, uO, cz, ad, av, cF, fS, aT, lG, dl, jo, af, eu, aW, EU, uC, ek, tk, ac, wg, hc, hR, wx, m); #c1(TCAIM) c2(NP_001025010) c3(T-cell activation inhibitor, mitochondrial isoform 2) c4(29193) c5(29194, 29195, 29196, 29197, 29198, 29199, 29200, 29201, 29202, 29203) c6( ) c7(cr); #c1(TCAIM) c2(NP_776187) c3(T-cell activation inhibitor, mitochondrial isoform 1) c4(250946) c5(250947, 250948, 250949, 250950, 250951, 250952, 250953, 250954, 250955, 250956) c6( ) c7(cr); #c1(TCAP) c2(NP_003664) c3(telethonin) c4(140048) c5(140049, 140050, 140051, 140052, 140053, 140054, 140055) c6( ) c7(cKC, AY, cc, aqZ, aW, xs, mx, km, aT, vc, cKB, mo); #c1(TCEA1) c2(NP_006747) c3(transcription elongation factor A protein 1 isoform 1) c4(167660, 167661) c5(167662, 167663, 167664, 167665, 167666, 167667) c6( ) c7(T, aE, c, aW, Y, aT); #c1(TCEA1) c2(NP_958845) c3(transcription elongation factor A protein 1 isoform 2) c4(262273) c5(262274, 262275, 262276, 262277, 262278, 262279) c6( ) c7(T, aE, c, aW, Y, aT); #c1(TCEA1) c2(XP_011515881) c3(transcription elongation factor A protein 1 isoform X2) c4( ) c5(308857, 308858, 308859, 308860, 308861, 308862) c6( ) c7(T, aE, c, aW, Y, aT); #c1(TCEA2) c2(NP_003186) c3(transcription elongation factor A protein 2 isoform a) c4(136107, 136108, 136109, 136110) c5(136111, 136112, 136113, 136114, 136115, 136116, 136117, 136118, 136119, 136120, 136121, 136122, 136123, 136124, 136125, 136126, 136127) c6( ) c7(aW, T, aE, bt, aT); #c1(TCEA2) c2(NP_942016) c3(transcription elongation factor A protein 2 isoform b) c4( ) c5(260429, 260430, 260431, 260432, 260433, 260434, 260435, 260436, 260437, 260438, 260439, 260440, 260441, 260442, 260443, 260444, 260445, 260446, 260447, 260448, 260449, 260450, 260451) c6( ) c7(aW, T, aE, bt, aT); #c1(TCEA2) c2(XP_005260286) c3(transcription elongation factor A protein 2 isoform X1) c4(276512, 276513) c5( ) c6( ) c7(aW, T, aE, bt, aT); #c1(TCEA3) c2(XP_011540358) c3(transcription elongation factor A protein 3 isoform X5) c4( ) c5(338818, 338819, 338820, 338821, 338822, 338823) c6( ) c7(aW, T, aE, aT); #c1(TCEAL1) c2(NP_001006641) c3(transcription elongation factor A protein-like 1) c4(22294, 22295) c5(22296, 22297, 22298, 22299, 22300, 22301, 22302, 22303, 22304, 22305, 22306, 22307, 22308, 22309, 22310, 22311, 22312, 22313, 22314, 22315, 22316) c6( ) c7(hd, id, nF, hl, at, dL, qN, t); #c1(TCEAL2) c2(NP_525129) c3(transcription elongation factor A protein-like 2) c4(231231) c5(231232, 231233, 231234, 231235, 231236, 231237, 231238, 231239, 231240, 231241) c6( ) c7(an); #c1(TCEAL4) c2(NP_001287830) c3 (transcription elongation factor A protein-like 4 isoform 5) c4(112944, 112945, 112946, 112947, 112948, 112949) c5(112950, 112951, 112952, 112953, 112954, 112955, 112956, 112957, 112958, 112959, 112960, 112961, 112962, 112963, 112964, 112965, 112966) c6( ) c7(T, hd, ci, nB); #c1(TCEAL7) c2(NP_689491) c3(transcription elongation factor A protein-like 7) c4(242884) c5(242885, 242886, 242887, 242888, 242889, 242890, 242891, 242892, 242893, 242894) c6( ) c7(T, Jl, aA, aF, m, cl, t, ad); #c1(TCEB1) c2(NP_001191793) c3 (transcription elongation factor B polypeptide 1 isoform b) c4(81763) c5(81764, 81765, 81766, 81767, 81768) c6( ) c7(aW, VB, aT, h, fr, ci, ae, LS, ad); #c1(TCEB1) c2(XP_011515883) c3(transcription elongation factor B polypeptide 1 isoform X1) c4(308863, 308864) c5(308865, 308866, 308867, 308868, 308869, 308870, 308871, 308872, 308873, 308874, 308875, 308876, 308877, 308878, 308879, 308880, 308881, 308882, 308883, 308884, 308885, 308886, 308887, 308888, 308889, 308890, 308891, 308892, 308893, 308894, 308895, 308896, 308897) c6( ) c7(aW, VB, aT, h, fr, ci, ae, LS, ad); #c1(TCEB2) c2(NP_009039) c3(transcription elongation factor B polypeptide 2 isoform a) c4(170363) c5(170364, 170365, 170366, 170367, 170368, 170369) c6( ) c7(aW, Rl, B, VB); #c1(TCEB2) c2(NP_996896) c3(transcription elongation factor B polypeptide 2 isoform b) c4(264857) c5(264858, 264859, 264860, 264861, 264862, 264863) c6( ) c7(aW, Rl, B, VB); #c1(TCEB3C) c2(NP_663628) c3(RNA polymerase II transcription factor SIII subunit A3) c4(241361) c5(241362, 241363, 241364, 241365, 241366, 241367) c6( ) c7(pX); #c1(TCERG1) c2(NP_001035095) c3(transcription elongation regulator 1 isoform 2) c4(32496) c5(32497, 32498, 32499, 32500, 32501, 32502) c6( ) c7(rR, hp); #c1(TCERG1) c2(NP_006697) c3(transcription elongation regulator 1 isoform 1) c4(167227) c5(167228, 167229, 167230, 167231, 167232, 167233, 167234) c6( ) c7(rR, hp); #c1(TCERG1L) c2(NP_777597) c3(transcription elongation regulator 1-like protein) c4(251293) c5(251294, 251295, 251296, 251297, 251298, 251299) c6( ) c7(bL, T, ac, bE, r, bB, aj, v, ci, q, J); #c1(TCF12) c2(NP_003196) c3(transcription factor 12 isoform b) c4(136193, 136194) c5(136195, 136196, 136197, 136198, 136199, 136200, 136201, 136202, 136203, 136204, 136205, 136206, 136207, 136208, 136209, 136210, 136211) c6( ) c7(x, aNA, cKD, wL, r, k, agj, an, cm, q, zV); #c1(TCF12) c2(NP_996920) c3(transcription factor 12 isoform a) c4(264896) c5(264897, 264898, 264899, 264900, 264901, 264902, 264903, 264904, 264905, 264906, 264907, 264908) c6( ) c7(x, aNA, cKD, wL, r, k, agj, an, cm, q, zV); #c1(TCF12) c2(NP_996923) c3(transcription factor 12 isoform c) c4(264909) c5(264910, 264911, 264912, 264913, 264914, 264915) c6( ) c7(x, aNA, cKD, wL, r, k, agj, an, cm, q, zV); #c1(TCF12) c2(XP_005254670) c3(transcription factor 12 isoform X5) c4(273296) c5( ) c6( ) c7(x, aNA, cKD, wL, r, k, agj, an, cm, q, zV); #c1(TCF12) c2(XP_011520263) c3(transcription factor 12 isoform X2) c4(315099, 315100) c5( ) c6( ) c7(x, aNA, cKD, wL, r, k, agj, an, cm, q, zV); #c1(TCF12) c2(XP_011520268) c3(transcription factor 12 isoform X8) c4( ) c5(315101, 315102, 315103, 315104, 315105, 315106, 315107, 315108, 315109, 315110, 315111, 315112) c6( ) c7(x, aNA, cKD, wL, r, k, agj, an, cm, q, zV); #c1(TCF15) c2(NP_004600) c3(transcription factor 15) c4(148054) c5(148055, 148056, 148057, 148058, 148059, 148060, 148061) c6( ) c7(kl); #c1(TCF19) c2(XP_005249391) c3(transcription factor 19 isoform X1) c4(269274, 269275) c5(269276, 269277, 269278, 269279, 269280, 269281, 269282, 269283, 269284, 269285, 269286, 269287, 269288, 269289, 269290, 269291, 269292) c6( ) c7(mj, A, cr, eZ, v, K, jE, hH, V, X); #c1(TCF19) c2(XP_005272924) c3(transcription factor 19 isoform X1) c4(284233) c5(284234, 284235, 284236, 284237, 284238, 284239, 284240, 284241, 284242, 284243, 284244, 284245, 284246, 284247, 284248, 284249, 284250, 284251, 284252, 284253, 284254, 284255, 284256, 284257, 284258, 284259, 284260, 284261, 284262, 284263, 284264, 284265, 284266, 284267, 284268, 284269, 284270, 284271, 284272, 284273, 284274, 284275, 284276, 284277, 284278, 284279, 284280, 284281) c6( ) c7(mj, A, cr, eZ, v, K, jE, hH, V, X); #c1(TCF19) c2(XP_005275645) c3(transcription factor 19 isoform X1) c4(285688) c5( ) c6( ) c7(mj, A, cr, eZ, v, K, jE, hH, V, X); #c1(TCF20) c2(NP_005641) c3(transcription factor 20 isoform 1) c4(158206) c5(158207, 158208, 158209, 158210, 158211, 158212) c6( ) c7(bL, asb, v, ad, aT); #c1(TCF20) c2(NP_852469) c3(transcription factor 20 isoform 2) c4(254998) c5(254999, 255000, 255001, 255002, 255003, 255004) c6( ) c7(bL, asb, v, ad, aT); #c1(TCF21) c2(NP_003197) c3(transcription factor 21) c4(136212) c5(136213, 136214, 136215, 136216, 136217, 136218, 136219, 136220, 136221, 136222) c6( ) c7(T, dD, ok, Dy, eM, ea, Y, ff, iw, iv, hy, j, ci, en, eq, g, ik, bz, cp); #c1(TCF25) c2(NP_055787) c3(transcription factor 25) c4(183499) c5(183500, 183501, 183502, 183503, 183504, 183505, 183506, 183507, 183508, 183509, 183510, 183511, 183512) c6( ) c7(ac); #c1(TCF25) c2(XP_005256355) c3 (transcription factor 25 isoform X4) c4(274120) c5(274121, 274122, 274123, 274124, 274125, 274126) c6( ) c7(ac); #c1(TCF25) c2(XP_011521262) c3(transcription factor 25 isoform X8) c4(316171) c5( ) c6( ) c7(ac); #c1(TCF3) c2(NP_001129611) c3(transcription factor E2-alpha isoform E47) c4(52012) c5(52013, 52014, 52015, 52016, 52017, 52018, 52019, 52020, 52021, 52022) c6( ) c7(T, wu, jh, fZ, aA, hF, q, ad, x, OG, TK, aT, hn, wm, pk, ae, azh, aW, Y, cr, r, af, cm, ck, ou, eU, QG, eD, Hb, m, k); #c1(TCF3) c2(NP_003191) c3(transcription factor E2-alpha isoform E(2) c4(136133) c5(136134, 136135, 136136, 136137, 136138, 136139) c6( ) c7(T, wu, jh, fZ, aA, hF, q, ad, x, OG, TK, aT, hn, wm, pk, ae, azh, aW, Y, cr, r, aF, cm, ck, ou, eU, QG, eD, Hb, m, k); #c1(TCF3) c2(XP_011526524) c3(transcription factor E2-alpha isoform X6) c4(320820) c5(320821, 320822, 320823, 320824, 320825, 320826) c6( ) c7(T, wu, jh, fZ, aA, hF, q, ad, x, OG, TK, aT, hn, wm, pk, ae, azh, aW, Y, cr, r, af, cm, ck, ou, eL, QG, eD, Hb, m, k); #c1(TCF3) c2(XP_011526525) c3(transcription factor E2-alpha isoform X7) c4( ) c5(320827) c6( ) c7(T, wu, jh, fZ, aA, hF, q, ad, x, OG, TK, aT, hn, wm, pk, ae, azh, aW, Y, cr, r, aF, cm, ck, ou, eL, QG, eD, Hb, m, k); #c1(TCF3) c2(XP_011526529) c3(transcription factor E2-alpha isoform X10) c4(320828) c5(320829, 320830, 320831, 320832, 320833) c6( ) c7(T, wu, jh, fZ, aA, hF, q, ad, x, OG, TK, aT, hn, wm, pk, ae, azh, aW, Y, cr, r, aF, cm, ck, ou, eU, QG, eD, Hb, m, k); #c1(TCF4) c2(NP_001230155) c3(transcription factor 4 isoform c) c4(86000, 86001, 86002) c5(86003, 86004, 86005, 86006, 86007, 86008, 86009, 86010, 86011, 86012, 86013, 86014, 86015, 86016, 86017, 86018, 86019) c6( ) c7(bL, xN, T, iV, bkx, atS, NT, aA, bB, mS, fr, ci, nA, bX, aQK, pz, q, ad, eA, eW, x, iu, hd, alu, Yc, hb, mW, aT, h, bK, K, mY, mV, ahE, Kq, he, KV, Qe, ae, mU, dF, Lu, d, B, s, aW, Y, Yd, Az, r, fe, gl, hN, aq, aF, ahl, g, cl, Z, t, QK, fs, mZ, bz, as, mX, ck, es, bF, iA, dB, bE, bmD, agl, cKE, gX, gZ, at, np, m, yA, lw, mT); #c1(TCF4) c2(NP_001230160) c3(transcription factor 4 isoform g) c4(86020) c5(86021, 86022, 86023, 86024, 86025, 86026) c6( ) c7(bL, xN, T, iV, bkx, atS, NT, aA, bB, mS, fr, ci, nA, bX, aQK, pz, q, ad, eA, eW, x, iu, hd, alu, Yc, hb, mW, aT, h, bK, K, mY, mV, ahE, Kq, he, KV, De, ae, mU, dF, Lu, d, B, s, aW, Y, Yd, Az, r, fe, gl, hN, aq, aF, ahl, g, cl, Z, t, DK, fs, mZ, bz, as, mX, ck, es, bF, iA, dB, bE, bmD, agl, cKE, gX, gZ, at, np, m, yA, lw, mT); #c1(TCF4) c2(NP_001230161) c3(transcription factor 4 isoform h) c4(86027) c5(86028, 86029, 86030, 86031, 86032, 86033) c6( ) c7(bL, xN, T, iV, bkx, atS, NT, aA, bB, mS, fr, ci, nA, bX, aQK, pz, q, ad, eA, eW, x, iu, hd, alu, Yc, hb, mW, aT, h, bK, K, mY, mV, ahE, Kq, he, KV, De, ae, ml, dF, Lu, d, B, s, aW, Y, Yd, Az, r, fe, gl, hN, aq, aF, ahl, g, cl, Z, t, QK, fs, mZ, bz, as, mX, ck, es, bF, iA, dB, bE, bmD, agl, cKE, gX, gZ, at, np, m, yA, lw, mT); #c1(TCF4) c2(NP_001230163) c3(transcription factor 4 isoform j) c4(86034) c5(86035, 86036, 86037, 86038, 86039, 86040) c6( ) c7(bL, xN, T, iV, bkx, atS, NT, aA, bB, mS, fr, ci, nA, bX, aQK, pz, q, ad, eA, eW, x, iu, hd, alu, Yc, hb, mW, aT, h, bK, K, mY, mV, ahE, Kq, he, KV, De, ae, mU, dF, Lu, d, B, s, aW, Y, Yd, Az, r, fe, gl, hN, aq, aF, ahl, g, cl, Z, t, QK, fs, mZ, bz, as, mX, ck, es, bF, iA, dB, bE, bmD, agl, cKE, gX, gZ, at, np, m, yA, lw, mT); #c1(TCF4) c2(NP_003190) c3(transcription factor 4 isoform b) c4( ) c5(136128, 136129, 136130, 136131, 136132) c6( ) c7(bL, xN, T, iV, bkx, atS, NT, aA, bB, mS, fr, ci, nA, bX, aQK, pz, q, ad, eA, eW, x, iu, hd, alu, Yc, hb, mW, aT, h, bK, K, mY, mV, ahE, Kq, he, KV, de, ae, mU, dF, Lu, d, B, s, aW, Y, Yd, Az, r, fe, gl, hN, aq, aF, ahl, g, cl, Z, t, QK, fs, mZ, bz, as, mX, ck, es, bF, iA, dB, bE, bmD, agl, cKE, gX, gZ, at, np, m, yA, lw, mT); #c1(TCF4) c2(XP_011524458) c3 (transcription factor 4 isoform X6) c4(319049, 319050) c5(319051) c6( ) c7(bL, xN, T, iV, bkx, atS, NT, aA, bB, mS, fr, ci, nA, bX, aQK, pz, q, ad, eA, eW, x, iu, hd, alu, Yc, hb, mW, aT, h, bK, K, mY, mV, ahE, Kq, he, KV, De, ae, mU, dF, Lu, d, B, s, aW, Y, Yd, Az, r, fe, gl, hN, aq, aF, ahl, g, cl, Z, t, QK, fs, mZ, bz, as, mX, ck, es, bF, iA, dB, bE, bmD, agl, cKE, gX, gZ, at, np, m, yA, lw, mT); #c1(TCF4) c2(XP_011524459) c3(transcription factor 4 isoform X7) c4(319052) c5( ) c6( ) c7(bL, xN, T, iV, bkx, atS, NT, aA, bB, mS, fr, ci, nA, bX, aQK, pz, q, ad, eA, eW, x, iu, hd, alu, Yc, hb, mW, aT, h, bK, K, mY, mV, ahE, Kq, he, KV, De, ae, mU, dF, Lu, d, B, s, aW, Y, Yd, Az, r, fe, gl, hN, aq, aF, ahl, g, cl, Z, t, QK, fs, mZ, bz, as, mX, ck, es, bF, iA, dB, bE, bmD, agl, cKE, gX, gZ, at, np, m, yA, lw, mT); #c1(TCF4) c2(XP_011524460) c3(transcription factor 4 isoform X9) c4( ) c5(319053) c6( ) c7(bL, xN, T, iV, bkx, atS, NT, aA, bB, mS, fr, ci, nA, bX, aQK, pz, q, ad, eA, eW, x, iu, hd, alu, Yc, hb, mW, aT, h, bK, K, mY, mV, ahE, Kq, he, KV, De, ae, mU, dF, Lu, d, B, s, aW, Y, Yd, Az, r, fe, gl, hN, aq, aF, ahl, g, cl, Z, t, QK, fs, mZ, bz, as, mX, ck, es, bF, iA, dB, bE, bmD, agl, cKE, gX, gZ, at, np, m, yA, lw, mT); #c1(TCF7) c2(NP_001128323) c3(transcription factor 7 isoform 3) c4(49835) c5(49836, 49837, 49838, 49839, 49840, 49841) c6( ) c7(bL, bM, T, bqP, jh, aA, bB, aj, j, ci, q, J, JK, bKm, eu, A, s, r, aF, cM, bF, ac, bE, bo, mf, k, B); #c1(TCF7) c2(NP_003193) c3(transcription factor 7 isoform 1) c4(136154, 136155) c5(136156, 136157, 136158, 136159, 136160, 136161, 136162, 136163) c6( ) c7(bL, bM, T, bqP, jh, aA, bB, aj, j, ci, q, J, JK, bKm, eu, A, s, r, aF, cM, bF, ac, bE, bo, mf, k, B); #c1(TCF7) c2(NP_963965) c3(transcription factor 7 isoform 4) c4( ) c5(262591, 262592, 262593, 262594, 262595, 262596, 262597, 262598, 262599, 262600, 262601, 262602, 262603, 262604, 262605, 262606, 262607, 262608, 262609, 262610) c6( ) c7(bL, bM, T, bqP, jh, aA, bB, aj, j, ci, q, J, JK, bKm, eu, A, s, r, aF, cM, bF, ac, bE, bo, mf, k, B); #c1(TCF7) c2(NP_998813) c3(transcription factor 7 isoform 2) c4( ) c5(266020, 266021, 266022, 266023, 266024) c6( ) c7(bL, bM, T, bqP, jh, aA, bB, aj, j, ci, q, J, JK, bKm, eu, A, s, r, aF, cM, bF, ac, bE, bo, mf, k, B); #c1(TCF7) c2(XP_006714742) c3(transcription factor 7 isoform X2) c4(288056, 288057, 288058, 288059) c5(288060, 288061, 288062, 288063, 288064, 288065, 288066, 288067, 288068, 288069, 288070, 288071, 288072, 288073) c6( ) c7(bL, bM, T, bqP, jh, aA, bB, aj, j, ci, q, J, JK, bKm, eu, A, s, r, aF, cM, bF, ac, bE, bo, mf, k, B); #c1(TCF7) c2(XP_006714747) c3(transcription factor 7 isoform X8) c4(288074, 288075, 288076, 288077, 288078) c5(288079, 288080, 288081, 288082, 288083, 288084, 288085, 288086, 288087, 288088, 288089, 288090, 288091, 288092, 288093, 288094, 288095, 288096, 288097, 288098) c6( ) c7(bL, bM, T, bqP, jh, aA, bB, aj, j, ci, q, J, JK, bKm, eu, A, s, r, aF, cM, bF, ac, bE, bo, mf, k, B); #c1(TCF7L1) c2(NP_112573) c3(transcription factor 7-like 1) c4(221442) c5(221443, 221444, 221445, 221446, 221447, 221448) c6( ) c7(ck, x, ou, T, QG, nB, aW, aT, fZ, aA, pk, aF, bK, m, k, bX, cm); #c1(TCF7L1) c2(XP_006712172) c3(transcription factor 7-like 1 isoform X1) c4( ) c5(286493) c6( ) c7(ck, x, ou, T, QG, nB, aW, aT, fZ, aA, pk, aF, bK, m, k, bX, cm); #c1(TCF7L2) c2(NP_001139746) c3(transcription factor 7-like 2 isoform 1) c4(59822, 59823) c5(59824, 59825, 59826, 59827, 59828, 59829) c6( ) c7(T, bkx, ea, e, bB, ci, aQK, x, K, dQ, bhm, dF, Qe, aW, fc, hl, g, Eg, as, iA, cKE, vy, k, m, lS, iu, xN, dD, yG, NT, aj, fr, nA, ml, eW, RD, Yc, h, jy, he, KV, vx, A, Yd, fe, aq, fs, W, dB, Fi, agl, mg, hL, B, bL, jv, iH, aA, cz, v, j, bX, q, J, eA, aPW, ga, hb, aT, bK, ahE, ae, af, Lu, kl, s, Y, r, ahl, cl, t, ck, ac, gX, mf, np, dy, yv, aZ, LX, dG, eq, pz, ad, av, hd, py, NS, Kq, uX, d, lR, cW, zR, gl, af, iV, iR, bz, dr, bF, es, bE, bmD, ei, X); #c1(TCF7L2) c2(NP_001139755) c3(transcription factor 7-like 2 isoform 3) c4(59881) c5(59882, 59883, 59884, 59885, 59886, 59887, 59888, 59889, 59890, 59891, 59892, 59893) c6( ) c7(T, bkx, ea, e, bB, ci, aQK, x, K, dQ, bhm, dF, Qe, aW, fc, hl, g, Eg, as, iA, cKE, vy, k, m, lS, iu, xN, dD, yG, NT, aj, fr, nA, ml, eW, RD, Yc, h, jy, he, KV, vx, A, Yd, fe, aq, fs, W, dB, Fi, agl, mg, hL, B, bL, jv, iH, aA, cz, v, j, bX, q, J, eA, aPW, ga, hb, aT, bK, ahE, ae, af, Lu, kl, s, Y, r, ahl, cl, t, ck, ac, gX, mf, np, dy, yv, aZ, LX, dG, eq, pz, ad, av, hd, py, NS, Kq, uX, d, lR, cW, zR, gl, aF, iV, iR, bz, dr, bF, es, bE, bmD, ei, X); #c1(TCF7L2) c2(NP_001139757) c3(transcription factor 7-like 2 isoform 5) c4(59894) c5(59895, 59896, 59897, 59898, 59899, 59900) c6( ) c7(T, bkx, ea, e, bB, ci, aQK, x, K, dd, bhm, dF, Qe, aW, fc, hl, g, Eg, as, iA, cKE, vy, k, m, lS, iu, xN, dD, yG, NT, aj, fr, nA, ml, eW, RD, Yc, h, jy, he, KV, vx, A, Yd, fe, aq, fs, W, dB, Fi, agl, mg, hL, B, bL, jv, iH, aA, cz, v, j, bX, q, J, eA, aPW, ga, hb, aT, bK, ahE, ae, af, Lu, kl, s, Y, r, ahl, cl, t, ck, ac, gX, mf, np, dy, yv, aZ, LX, dG, eq, pz, ad, av, hd, py, NS, Kq, uX, d, lR, cW, zR, gl, aF, iV, iR, bz, dr, bF, es, bE, bmD, ei, X); #c1(TCF7L2) c2(NP_001185457) c3(transcription factor 7-like 2 isoform 10) c4( ) c5(75613, 75614, 75615, 75616, 75617, 75618) c6( ) c7(T, bkx, ea, e, bB, ci, aQK, x, K, dQ, bhm, dF, Qe, aW, fc, hl, g, Eg, as, iA, cKE, vy, k, m, lS, iu, xN, dD, yG, NT, aj, fr, nA, ml, eW, RD, Yc, h, jy, he, KV, vx, A, Yd, fe, aq, fs, W, dB, Fi, agl, mg, hL, B, bL, jv, iH, aA, cz, v, j, bX, q, J, eA, aPW, ga, hb, aT, bK, ahE, ae, af, Lu, kl, s, Y, r, ahl, cl, t, ck, ac, gX, mf, np, dy, yv, aZ, LX, dG, eq, pz, ad, av, hd, py, NS, Kq, uX, d, lR, cW, zR, gl, aF, iV, iR, bz, dr, bF, es, bE, bmD, ei, X); #c1(TCF7L2) c2(NP_001185459) c3(transcription factor 7-like 2 isoform 12) c4(75619) c5(75620, 75621, 75622, 75623, 75624, 75625) c6( ) c7(T, bkx, ea, e, bB, ci, aQK, x, K, dQ, bhm, dF, Qe, aW, fc, hl, g, Eg, as, iA, cKE, vy, k, m, lS, iu, xN, dD, yG, NT, aj, fr, nA, ml, eW, RD, Yc, h, jy, he, KV, vx, A, Yd, fe, aq, fs, W, dB, Fi, agl, mg, hL, B, bL, jv, iH, aA, cz, v, j, bX, q, J, eA, aPW, ga, hb, aT, bK, ahE, ae, af, Lu, kl, s, Y, r, ahl, cl, t, ck, ac, gX, mf, np, dy, yv, aZ, LX, dG, eq, pz, ad, av, hd, py, NS, Kq, uX, d, lR, cW, zR, gl, aF, iV, iR, bz, dr, bF, es, bE, bmD, ei, X); #c1(TCF7L2) c2(NP_110383) c3(transcription factor 7-like 2 isoform 2) c4(219841, 219842) c5(219843, 219844, 219845, 219846, 219847, 219848, 219849) c6( ) c7(T, bkx, ea, e, bB, ci, aQK, x, K, dQ, bhm, dF, Qe, aW, fc, hl, g, Eg, as, iA, cKE, vy, k, m, lS, iu, xN, dD, yG, NT, aj, fr, nA, ml, eW, RD, Yc, h, jy, he, KV, vx, A, Yd, fe, aq, fs, W, dB, Fi, agl, mg, hL, B, bL, jv, iH, aA, cz, v, j, bX, q, J, eA, aPW, ga, hb, aT, bK, ahE, ae, af, Lu, kl, s, Y, r, ahl, cl, t, ck, ac, gX, mf, np, dy, yv, aZ, LX, dG, eq, pz, ad, av, hd, py, NS, Kq, uX, d, lR, cW, zR, gl, aF, iV, iR, bz, dr, bF, es, bE, bmD, ei, X); #c1(TCF7L2) c2(XP_005270137) c3(transcription factor 7-like 2 isoform X9) c4(283144) c5( ) c6( ) c7(T, bkx, ea, e, bB, ci, aQK, x, K, dQ, bhm, dF, Qe, aW, fc, hl, g, Eg, as, iA, cKE, vy, k, m, lS, iu, xN, dD, yG, NT, aj, fr, nA, ml, eW, RD, Yc, h, jy, he, KV, vx, A, Yd, fe, aq, fs, W, dB, Fi, agl, mg, hL, B, bL, jv, iH, aA, cz, v, j, bX, q, J, eA, aPW, ga, hb, aT, bK, ahE, ae, af, Lu, kl, s, Y, r, ahl, cl, t, ck, ac, gX, mf, np, dy, yv, aZ, LX, dG, eq, pz, ad, av, hd, py, NS, Kq, uX, d, lR, cW, zR, gl, aF, iV, iR, bz, dr, bF, es, bE, bmD, ei, X); #c1(TCF7L2) c2(XP_005270141) c3(transcription factor 7-like 2 isoform X13) c4(283145, 283146, 283147) c5(283148, 283149, 283150, 283151, 283152, 283153, 283154, 283155, 283156, 283157, 283158, 283159, 283160, 283161, 283162, 283163, 283164, 283165, 283166, 283167, 283168, 283169, 283170, 283171) c6( ) c7(T, bkx, ea, e, bB, ci, aQK, x, K, dQ, bhm, dF, Qe, aW, fc, hl, g, Eg, as, iA, cKE, vy, k, m, lS, iu, xN, dD, yG, NT, aj, fr, nA, ml, eW, RD, Yc, h, jy, he, KV, vx, A, Yd, fe, aq, fs, W, dB, Fi, agl, mg, hL, B, bL, jv, iH, aA, cz, v, j, bX, q, J, eA, aPW, ga, hb, aT, bK, ahE, ae, af, Lu, kl, s, Y, r, ahl, cl, t, ck, ac, gX, mf, np, dy, yv, aZ, LX, dG, eq, pz, ad, av, hd, py, NS, Kq, uX, d, lR, cW, zR, gl, aF, iV, iR, bz, dr, bF, es, bE, bmD, ei, X); #c1(TCF7L2) c2(XP_005270143) c3(transcription factor 7-like 2 isoform X15) c4(283172) c5(283173) c6( ) c7(T, bkx, ea, e, bB, ci, aQk, x, K, dl, bhm, dF, de, aW, fc, hl, g, Eg, as, iA, cKE, vy, k, m, lS, iu, xN, dD, yG, NT, aj, fr, nA, ml, eW, RD, Yc, h, jy, he, KV, vx, A, Yd, fe, aq, fs, W, dB, Fi, agl, mg, hL, B, bL, jv, iH, aA, cz, v, j, bX, q, J, eA, aPW, ga, hb, aT, bK, ahE, ae, af, Lu, kl, s, Y, r, ahl, cl, t, ck, ac, gX, mf, np, dy, yv, aZ, LX, dG, eq, pz, ad, av, hd, py, NS, Kq, uX, d, lR, cW, zR, gl, aF, iV, iR, bz, dr, bF, es, bE, bmD, ei, X); #c1(TCF7L2) c2(XP_005270151) c3 (transcription factor 7-like 2 isoform X24) c4(283174) c5(283175, 283176, 283177, 283178, 283179, 283180) c6( ) c7(T, bkx, ea, e, bB, ci, aQK, x, K, dl, bhm, dF, Qe, aW, fc, hl, g, Eg, as, iA, cKE, vy, k, m, lS, iu, xN, dD, yG, NT, aj, fr, nA, ml, eW, RD, Yc, h, jy, he, KV, vx, A, Yd, fe, aq, fs, W, dB, Fi, agl, mg, hL, B, bL, jv, iH, aA, cz, v, j, bX, q, J, eA, aPW, ga, hb, aT, bK, ahE, ae, af, Lu, kl, s, Y, r, ahl, cl, t, ck, ac, gX, mf, np, dy, yv, aZ, LX, dG, eq, pz, ad, av, hd, py, NS, Kq, uX, d, lR, cW, zR, gl, aF, iV, iR, bz, dr, bF, es, bE, bmD, ei, X); #c1(TCF7L2) c2(XP_005270153) c3(transcription factor 7-like 2 isoform X31) c4(283181) c5(283182, 283183, 283184, 283185, 283186, 283187) c6( ) c7(T, bkx, ea, e, bB, ci, aQK, x, K, dQ, bhm, dF, Qe, aW, fc, hl, g, Eg, as, iA, cKE, vy, k, m, lS, iu, xN, dD, yG, NT, aj, fr, nA, ml, eW, RD, Yc, h, jy, he, KV, vx, A, Yd, fe, aq, fs, W, dB, Fi, agl, mg, hL, B, bL, jv, iH, aA, cz, v, j, bX, q, J, eA, aPW, ga, hb, aT, bK, ahE, ae, af, Lu, kl, s, Y, r, ahl, cl, t, ck, ac, gX, mf, np, dy, yv, aZ, LX, dG, eq, pz, ad, av, hd, py, NS, Kq, uX, d, lR, cW, zR, gl, aF, iV, iR, bz, dr, bF, es, bE, bmD, ei, X); #c1(TCF7L2) c2(XP_005270159) c3(transcription factor 7-like 2 isoform X34) c4(283188) c5(283189, 283190, 283191, 283192, 283193, 283194) c6( ) c7(T, bkx, ea, e, bB, ci, aQK, x, K, dQ, bhm, dF, de, aW, fc, hl, g, Eg, as, iA, cKE, vy, k, m, lS, iu, xN, dD, yG, NT, aj, fr, nA, ml, eW, RD, Yc, h, jy, he, KV, vx, A, Yd, fe, aq, fs, W, dB, Fi, agl, mg, hL, B, bL, jv, iH, aA, cz, v, j, bX, q, J, eA, aPW, ga, hb, aT, bK, ahE, ae, af, Lu, kl, s, Y, r, ahl, cl, t, ck, ac, gX, mf, np, dy, yv, aZ, LX, dG, eq, pz, ad, av, hd, py, NS, Kq, uX, d, lR, cW, zR, gl, aF, iV, iR, bz, dr, bF, es, bE, bmD, ei, X); #c1(TCF7L2) c2(XP_006718019) c3(transcription factor 7-like 2 isoform X27) c4( ) c5(291460, 291461, 291462, 291463, 291464) c6( ) c7(T, bkx, ea, e, bB, ci, aQK, x, K, dQ, bhm, dF, Qe, aW, fc, hl, g, Eg, as, iA, cKE, vy, k, m, lS, iu, xN, dD, yG, NT, aj, fr, nA, ml, eW, RD, Yc, h, jy, he, KV, vx, A, Yd, fe, aq, fs, W, dB, Fi, agl, mg, hL, B, bL, jv, iH, aA, cz, v, j, bX, q, J, eA, aPW, ga, hb, aT, bK, ahE, ae, af, Lu, kl, s, Y, r, ahl, cl, t, ck, ac, gX, mf, np, dy, yv, aZ, LX, dG, eq, pz, ad, av, hd, py, NS, Kq, uX, d, lR, cW, zR, gl, aF, iV, iR, bz, dr, bF, es, bE, bmD, ei, X); #c1(TCF7L2) c2(XP_011538418) c3(transcription factor 7-like 2 isoform X25) c4(336099) c5(336100, 336101, 336102, 336103, 336104, 336105) c6( ) c7(T, bkx, ea, e, bB, ci, aQK, x, K, dQ, bhm, dF, Qe, aW, fc, hl, g, Eg, as, iA, cKE, vy, k, m, lS, iu, xN, dD, yG, NT, aj, fr, nA, ml, eW, RD, Yc, h, jy, he, KV, vx, A, Yd, fe, aq, fs, W, dB, Fi, agl, mg, hL, B, bL, jv, iH, aA, cz, v, j, bX, q, J, eA, aPW, ga, hb, aT, bk, ahE, ae, af, Lu, kl, s, Y, r, ahl, cl, t, ck, ac, gX, mf, np, dy, yv, aZ, LX, dG, eq, pz, ad, av, hd, py, NS, Kq, uX, d, lR, cW, zR, gl, aF, iV, iR, bz, dr, bF, es, bE, bmD, ei, X); #c1(TCFL5) c2(NP_006593) c3(transcription factor-like 5 protein isoform 1) c4(166505) c5(166506, 166507, 166508, 166509, 166510, 166511, 166512) c6( ) c7(x, d, ac, s, oz, il, aw, np, ci, k, bX, ao); #c1(TCFL5) c2(XP_005260241) c3(transcription factor-like 5 protein isoform X1) c4(276479) c5(276480, 276481, 276482, 276483, 276484, 276485) c6( ) c7(x, d, ac, s, oz, il, aw, np, ci, k, bX, ao); #c1(TCHH) c2(NP_009044) c3(trichohyalin) c4(170383) c5(170384, 170385, 170386, 170387, 170388, 170389, 170390, 170391) c6( ) c7(bdQ); #c1(TCHP) c2(XP_011537139) c3(trichoplein keratin filament-binding protein isoform X1) c4(334870, 334871, 334872) c5(334873, 334874, 334875, 334876, 334877, 334878, 334879, 334880, 334881, 334882, 334883, 334884, 334885, 334886, 334887, 334888, 334889, 334890, 334891, 334892, 334893, 334894, 334895, 334896, 334897, 334898) c6( ) c7(bL, T, hl, jW, pY, bui, if, jx, aA, bB, Fb, fr, ci, qN, q, cd, ad, er, eA, ip, oT, ft, cq, Rl, py, aMT, aT, K, jj, ae, pi, avQ, bF, bum, d, cH, aW, Y, Ls, eH, ff, r, aYp, aq, aF, yq, Ku, en, cl, t, eF, bz, cm, iR, oU, ck, kn, KT, sS, YE, es, eM, iA, dB, bE, bP, afd, lT, Ew, azg, m, zu, bt, kS, B, VB, cn); #c1(TCIRG1) c2(NP_006044) c3(V-type proton ATPase 116 kDa subunit a isoform 3 isoform b) c4(161433) c5( ) c6( ) c7(bL, XW, bsH, lD, zw, aiV, cnh, zs, wR, ajW, C, hh, bA, BY); #c1(TCIRG1) c2(XP_011543028) c3(V-type proton ATPase 116 kDa subunit a isoform 3 isoform X1) c4(341735, 341736) c5(341737, 341738, 341739, 341740, 341741, 341742, 341743, 341744, 341745, 341746, 341747, 341748) c6( ) c7(bL, XW, bsH, lD, zw, aiV, cnh, zs, wR, ajW, C, hh, bA, BY); #c1(TCIRG1) c2(XP_011543029) c3(V-type proton ATPase 116 kDa subunit a isoform 3 isoform X2) c4(341749, 341750) c5(341751, 341752, 341753, 341754, 341755, 341756, 341757, 341758, 341759, 341760, 341761, 341762) c6( ) c7(bL, XW, bsH, lD, zw, aiV, cnh, zs, wR, ajW, C, hh, bA, BY); #c1(TCIRG1) c2(XP_011543031) c3(V-type proton ATPase 116 kDa subunit a isoform 3 isoform X4) c4( ) c5(341763, 341764, 341765, 341766, 341767, 341768, 341769, 341770) c6( ) c7(bL, XW, bsH, lD, zw, aiV, cnh, zs, wR, ajW, C, hh, bA, BY); #c1(TCL1A) c2(NP_001092195) c3(T-cell leukemia/lymphoma protein 1A) c4(39424) c5(39425, 39426, 39427, 39428, 39429, 39430, 39431, 39432, 39433, 39434, 39435, 39436, 39437, 39438, 39439, 39440, 39441, 39442) c6( ) c7(oO, T, fM, wu, jh, fd, alq, cd, x, cc, KH, aT, K, wm, cKF, pk, on, aol, zQ, je, aW, oV, ho, bw, ck, iM, ac, beE, cj, k, bC, bt, B); #c1(TCL1B) c2(NP_004909) c3(T-cell leukemia/lymphoma protein 1B) c4(150711) c5(150712, 150713, 150714, 150715, 150716, 150717, 150718, 150719, 150720, 150721) c6( ) c7(T, fM, wu, jh, fd, alq, kH, aT, K, wm, cKF, pk, on, aol, je, aW, oV, ho, ck, iM, nB, beE, cj, bC, bt, B, L); #c1(TCN1) c2(NP_001053) c3(transcobalamin-1 precursor) c4(34416) c5(34417, 34418, 34419, 34420, 34421, 34422) c6( ) c7(T, dD, fM, iW, mN, j, eq, aQF, ad, h, ae, af, il, pl, f, fq, hq, pH, GG, UP, wE, au); #c1(TCN2) c2(NP_000346) c3(transcobalamin-2 isoform 1 precursor) c4(7618) c5(7619, 7620, 7621, 7622, 7623, 7624) c6( ) c7(dD, eR, fR, sQ, fV, e, jh, aA, sM, mN, v, j, eq, cKH, q, ad, ip, md, Na, ib, M, dV, Nc, lQ, avl, af, eu, Uf, d, s, r, Ll, Lk, g, iV, f, bN, apM, ac, UP, dB, lp, wE, bm, uo, ut, uJ, aS, cKG, ij, eg, bD, us); #c1(TCN2) c2(NP_001171655) c3(transcobalamin-2 isoform 2 precursor) c4(71161) c5(71162, 71163, 71164, 71165, 71166, 71167) c6( ) c7(dD, eR, fR, sQ, fV, e, jh, aA, sM, mN, v, j, eq, cKH, q, ad, ip, md, Na, ib, M, dV, Nc, lQ, avl, af, eu, Uf, d, s, r, Ll, Lk, g, iV, f, bN, apM, ac, UP, dB, lp, wE, bm, uo, ut, uJ, aS, cKG, ij, eg, bD, us); #c1(TCOF1) c2(NP_000347) c3(treacle protein isoform b) c4(7625) c5(7626, 7627, 7628, 7629, 7630, 7631) c6( ) c7(agS, Na, aMU, Cq, yr, if, aMS, cC, Az, HN, Xg, fe, Km, K, nA, ahl, uF, qN, zW, ahE, Wt); #c1(TCOF1) c2(NP_001008657) c3 (treacle protein isoform c) c4(23338) c5(23339, 23340, 23341, 23342, 23343, 23344) c6( ) c7(agS, Na, aMU, Cq, yr, if, aMS, cC, Az, HN, Xg, fe, Km, K, nA, ahl, uF, qN, zW, ahE, Wt); #c1(TCOF1) c2(NP_001128715) c3(treacle protein isoform d) c4(50615) c5(50616, 50617, 50618, 50619, 50620, 50621) c6( ) c7(agS, Na, aMU, Cq, yr, if, aMS, cC, Az, HN, Xg, fe, Km, K, nA, ahl, uF, qN, zW, ahE, Wt); #c1(TCOF1) c2(NP_001128716) c3(treacle protein isoform e) c4(50622) c5(50623, 50624, 50625, 50626, 50627, 50628) c6( ) c7(agS, Na, aMU, Cq, yr, if, aMS, cC, Az, HN, Xg, fe, Km, K, nA, ahl, uF, qN, zW, ahE, Wt); #c1(TCOF1) c2(NP_001128717) c3(treacle protein isoform f) c4(50629) c5(50630, 50631, 50632, 50633, 50634, 50635) c6( ) c7(agS, Na, aMU, Cq, yr, if, aMS, cC, Az, HN, Xg, fe, Km, K, nA, ahl, uF, qN, zW, ahE, Wt); #c1(TCOF1) c2(NP_001182070) c3(treacle protein isoform g) c4(74552) c5(74553, 74554, 74555, 74556, 74557, 74558) c6( ) c7(agS, Na, aMU, Cq, yr, if, aMS, cC, Az, HN, Xg, fe, Km, K, nA, ahl, uF, qN, zW, ahE, Wt); #c1(TCOF1) c2(XP_005268561) c3 (treacle protein isoform X3) c4(281908) c5(281909, 281910, 281911, 281912, 281913, 281914) c6( ) c7(agS, Na, aMU, Cq, yr, if, aMS, cC, Az, HN, Xg, fe, Km, K, nA, ahl, uF, qN, zW, ahE, Wt); #c1(TCOF1) c2(XP_005268562) c3(treacle protein isoform X4) c4(281915) c5(281916, 281917, 281918, 281919, 281920, 281921) c6( ) c7(agS, Na, aMU, Cq, yr, if, aMS, cC, Az, HN, Xg, fe, Km, K, nA, ahl, uF, qN, zW, ahE, Wt); #c1(TCP1) c2(NP_001008897) c3(T-complex protein 1 subunit alpha isoform b) c4( ) c5(23483, 23484, 23485, 23486, 23487) c6( ) c7(qX, lf, r, f, q, ad); #c1(TCP1) c2(NP_110379) c3(T-complex protein 1 subunit alpha isoform a) c4(219796, 219797, 219798) c5(219799, 219800, 219801, 219802, 219803, 219804, 219805, 219806, 219807, 219808, 219809, 219810, 219811) c6( ) c7(qX, lf, r, f, q, ad); #c1(TCP10) c2(NP_004601) c3(T-complex protein 10A homolog) c4(148062) c5(148063, 148064, 148065, 148066, 148067) c6( ) c7(j); #c1(TCP10L2) c2(NP_001138593) c3(putative t-complex protein 10A homolog 2) c4(57(26) c5(57127, 57128, 57129, 57130, 57131, 57132, 57133) c6( ) c7(cz, pN); #c1(TCP11L1) c2(NP_060863) c3(T-complex protein 11-like protein 1) c4(198146, 198147, 198148) c5(198149, 198150, 198151, 198152, 198153, 198154, 198155, 198156, 198157, 198158, 198159, 198160, 198161, 198162, 198163, 198164, 198165, 198166, 198167, 198168) c6( ) c7(h); #c1(TCP11L1) c2(XP_011518508) c3(T-complex protein 11-like protein 1 isoform X2) c4(312602) c5( ) c6( ) c7(h); #c1(TCP11L1) c2(XP_011518510) c3(T-complex protein 11-like protein 1 isoform X3) c4( ) c5(312603, 312604, 312605, 312606, 312607, 312608, 312609, 312610, 312611, 312612, 312613, 312614) c6( ) c7(h); #c1(TCTA) c2(NP_071503) c3(T-cell leukemia translocation-altered gene protein) c4(211784) c5(211785, 211786, 211787, 211788, 211789, 211790) c6( ) c7(dL, kH); #c1(TCTE1) c2(XP_011512640) c3(T-complex-associated testis-expressed protein 1 isoform X1) c4(303607) c5(303608, 303609, 303610, 303611, 303612, 303613) c6( ) c7(akB); #c1(TCTE1) c2(XP_011512642) c3(T-complex-associated testis-expressed protein 1 isoform X4) c4(303614) c5(303615, 303616, 303617, 303618, 303619, 303620, 303621, 303622, 303623, 303624) c6( ) c7(akB); #c1(TCTN1) c2(NP_001076006) c3(tectonic-1 isoform 2 precursor) c4(37310) c5(37311, 37312, 37313, 37314, 37315, 37316, 37317, 37318) c6(37319, 37320) c7(cKl); #c1(TCTN1) c2(NP_001076007) c3(tectonic-1 isoform 1 precursor) c4(37321) c5(37322, 37323, 37324, 37325, 37326, 37327, 37328, 37329) c6(37330, 37331) c7(cKl); #c1(TCTN1) c2(NP_001167446) c3(tectonic-1 isoform 4) c4(68895) c5(68896, 68897, 68898, 68899, 68900, 68901, 68902, 68903, 68904, 68905) c6(68906, 68907) c7(cKl); #c1(TCTN1) c2(NP_001167447) c3(tectonic-1 isoform 5 precursor) c4(68908) c5(68909, 68910, 68911, 68912, 68913, 68914, 68915, 68916, 68917) c6(68918, 68919) c7(cKl); #c1(TCTN1) c2(NP_078825) c3(tectonic-1 isoform 3 precursor) c4(215622) c5(215623, 215624, 215625, 215626, 215627, 215628, 215629, 215630) c6(215631, 215632) c7(cKl); #c1(TCTN1) c2(XP_005253993) c3(tectonic-1 isoform X12) c4(272857) c5(272858, 272859, 272860, 272861, 272862, 272863, 272864) c6(272865, 272866) c7(cKl); #c1(TCTN1) c2(XP_011537036) c3(tectonic-1 isoform X2) c4(334798) c5(334799, 334800, 334801, 334802, 334803, 334804, 334805, 334806, 334807, 334808, 334809, 334810) c6(334811, 334812) c7(cKl); #c1(TCTN1) c2(XP_011537039) c3(tectonic-1 isoform X6) c4(334813) c5(334814, 334815, 334816, 334817, 334818, 334819, 334820, 334821) c6(334822, 334823) c7(cKl); #c1(TCTN1) c2(XP_011537041) c3(tectonic-1 isoform X8) c4(334824) c5( ) c6( ) c7(cKl); #c1(TCTN2) c2(NP_001137322) c3(tectonic-2 isoform 2 precursor) c4(55469) c5(55470, 55471, 55472, 55473, 55474, 55475) c6( ) c7(vm, ago, cKJ); #c1(TCTN2) c2(NP_079085) c3(tectonic-2 isoform 1 precursor) c4(216930) c5(216931, 216932, 216933, 216934, 216935, 216936) c6( ) c7(vm, ago, cKJ); #c1(TCTN3) c2(NP_001137445) c3(tectonic-3 isoform b precursor) c4(55758) c5(55759, 55760, 55761, 55762, 55763, 55764) c6( ) c7(bet, cKK, j); #c1(TCTN3) c2(NP_056446) c3(tectonic-3 isoform a precursor) c4(187580, 187581, 187582) c5(187583, 187584, 187585, 187586, 187587) c6( ) c7(bet, cKK, j); #c1(TCTN3) c2(XP_005269747) c3(tectonic-3 isoform X2) c4(282739) c5(282740, 282741, 282742, 282743, 282744, 282745) c6( ) c7(bet, cKK, j); #c1(TCTN3) c2(XP_011537929) c3(tectonic-3 isoform X1) c4( ) c5(335424, 335425, 335426, 335427, 335428, 335429, 335430) c6( ) c7(bet, cKK, j); #c1(TCTN3) c2(XP_011537930) c3(tectonic-3 isoform X3) c4( ) c5(335431, 335432, 335433, 335434, 335435, 335436) c6( ) c7(bet, cKK, j); #c1(TDG) c2(NP_003202) c3(G/T mismatch-specific thymine DNA glycosylase) c4(136223) c5(136224, 136225, 136226, 136227, 136228, 136229, 136230) c6( ) c7(av, T, bE, CT, aA, r, bB, dL, aF, V, q, hX); #c1(TDG) c2(XP_01537017) c3(G/T mismatch-specific thymine DNA glycosylase isoform X2) c4(334773) c5(334774, 334775, 334776, 334777, 334778) c6( ) c7(av, T, bE, CT, aA, r, bB, dL, aF, V, q, hX); #c1(TDGF1) c2(NP_001167607) c3(teratocarcinoma-derived growth factor 1 isoform 2) c4( ) c5(69409, 69410, 69411, 69412, 69413) c6( ) c7(T, acl, aA, bB, n, Hn, j, ci, Ea, cKL, q, eA, dP, aE, hW, aT, bA, wT, aW, Y, ff, r, en, bu, t, op, Dy, eM, bE, eD, vA, m, bD, ZW, X); #c1(TDGF1) c2(NP_003203) c3(teratocarcinoma-derived growth factor 1 isoform 1 precursor) c4(136231, 136232) c5(136233, 136234, 136235, 136236, 136237, 136238, 136239, 136240, 136241) c6( ) c7(T, acl, aA, bB, n, Hn, j, ci, Ea, cKL, q, eA, dP, aE, hW, aT, bA, vT, aW, Y, ff, r, en, bu, t, op, Dy, eM, bE, eD, vA, m, bD, ZW, X); #c1(TDO2) c2(NP_005642) c3(tryptophan 2,3-dioxygenase) c4(158213) c5(158214, 158215, 158216, 158217, 158218, 158219) c6( ) c7(tJ, T, cKM, bO, To, ti, bS, aqu, ct, gf, Gd, uu, qc, qi, rc, iX, aql, bma, aaw, bK, K, aqD, H, he, rz, bME, af, xf, aWm, yL, xs, zR, gl, Z, vo, uC, bN, hq, cA, dB, Ei, uJ, Fl, ra, pk, xg, agZ, L); #c1(TDP1) c2(XP_006720263) c3(tyrosyl-DNA phosphodiesterase 1 isoform X3) c4( ) c5(293329, 293330, 293331, 293332, 293333, 293334) c6( ) c7(gV, T, bv, c, kB, hG, eM, KH, cKN, U, bk, K, bC, kC, bz, aR); #c1(TDP1) c2(XP_011535245) c3(tyrosyl-DNA phosphodiesterase 1 isoform X1) c4(332108, 332109, 332110, 332111, 332112, 332113, 332114) c5(332115, 332116, 332117, 332118, 332119, 332120, 332121, 332122, 332123, 332124, 332125) c6( ) c7(gV, T, bv, c, kB, hG, eM, KH, cKN, U, bk, K, bC, kC, bz, aR); #c1(TDP1) c2(XP_011535246) c3(tyrosyl-DNA phosphodiesterase 1 isoform X2) c4(332126) c5(332127, 332128, 332129, 332130, 332131, 332132, 332133, 332134, 332135, 332136, 332137, 332138, 332139, 332140, 332141, 332142, 332143, 332144, 332145, 332146, 332147, 332148, 332149, 332150, 332151, 332152, 332153, 332154, 332155, 332156, 332157, 332158, 332159, 332160, 332161) c6( ) c7(gV, T, bv, c, kB, hG, eM, KH, cKN, U, bk, K, bC, kC, bz, aR); #c1(TDP2) c2(NP_057698) c3(tyrosyl-DNA phosphodiesterase 2) c4(192398, 192399, 192400, 192401) c5(192402, 192403, 192404, 192405, 192406, 192407, 192408, 192409, 192410, 192411, 192412, 192413, 192414, 192415, 192416, 192417, 192418, 192419, 192420, 192421, 192422, 192423, 192424, 192425) c6( ) c7(T, cF, eM, yW, afv, np, yA, bz); #c1(TDRD1) c2(NP_942090) c3(tudor domain-containing protein 1) c4(260459) c5(260460, 260461, 260462, 260463, 260464, 260465) c6( ) c7(n, ae, gU, ad); #c1(TDRD1) c2(XP_005270036) c3(tudor domain-containing protein 1 isoform X4) c4(283049, 283050) c5(283051, 283052, 283053, 283054, 283055, 283056, 283057, 283058, 283059, 283060, 283061, 283062) c6( ) c7(n, ae, gU, ad); #c1(TDRD1) c2(XP_011538266) c3(tudor domain-containing protein 1 isoform X3) c4(335895) c5(335896, 335897, 335898, 335899, 335900, 335901) c6( ) c7(n, ae, gU, ad); #c1(TDRD1) c2(XP_011538267) c3(tudor domain-containing protein 1 isoform X5) c4(335902) c5(335903, 335904, 335905, 335906, 335907, 335908) c6( ) c7(n, ae, gU, ad); #c1(TDRD3) c2(NP_001139542) c3 (tudor domain-containing protein 3 isoform 1) c4(59478, 59479) c5(59480, 59481, 59482, 59483, 59484, 59485, 59486, 59487, 59488, 59489) c6( ) c7(gv, K, Ku); #c1(TDRD3) c2(NP_110421) c3(tudor domain-containing protein 3 isoform 2) c4( ) c5(220245, 220246, 220247, 220248, 220249, 220250, 220251, 220252, 220253, 220254, 220255) c6( ) c7(gv, K, Ku); #c1(TDRD3) c2(XP_011533551) c3(tudor domain-containing protein 3 isoform X5) c4(329669) c5(329670, 329671, 329672, 329673, 329674) c6( ) c7(gv, K, Ku); #c1(TDRD5) c2(NP_001186020) c3(tudor domain-containing protein 5 isoform 2) c4(76876) c5(76877, 76878, 76879, 76880, 76881, 76882, 76883, 76884, 76885, 76886) c6( ) c7(cz); #c1(TDRD5) c2(XP_005244992) c3(tudor domain-containing protein 5 isoform X1) c4(266192, 266193) c5(266194, 266195, 266196, 266197, 266198, 266199, 266200, 266201, 266202, 266203, 266204, 266205) c6( ) c7(cz); #c1(TDRD6) c2(NP_001010870) c3(tudor domain-containing protein 6 isoform 1) c4(23863, 23864, 23865) c5(23866, 23867, 23868, 23869, 23870, 23871, 23872, 23873, 23874, 23875, 23876, 23877, 23878, 23879, 23880, 23881, 23882, 23883) c6( ) c7(bB, bE); #c1(TDRD6) c2(NP_001161831) c3(tudor domain-containing protein 6 isoform 2) c4(66114) c5(66115, 66116, 66117, 66118, 66119, 66120) c6( ) c7(bB, bE); #c1(TDRD7) c2(NP_001289813) c3(tudor domain-containing protein 7 isoform 2) c4( ) c5(114667, 114668, 114669, 114670, 114671, 114672) c6( ) c7(gU, aA, zh, fr, ze, zo, ad, ft, ib, aE, ds, dV, zg, cKO, ae, d, zb, ff, aF, en, dw, ve, t, bz, zr, zl, eg, zk); #c1(TDRD7) c2(NP_055105) c3(tudor domain-containing protein 7 isoform 1) c4(178977, 178978, 178979) c5(178980, 178981, 178982, 178983, 178984, 178985, 178986) c6( ) c7(gU, aA, zh, fr, ze, zo, ad, ft, ib, aE, ds, dV, zg, cKO, ae, d, zb, ff, aF, en, dw, ve, t, bz, zr, zl, eg, zk); #c1(TDRD7) c2(XP_011516726) c3(tudor domain-containing protein 7 isoform X2) c4( ) c5(310195, 310196, 310197, 310198, 310199) c6( ) c7(gU, aA, zh, fr, ze, zo, ad, ft, ib, aE, ds, dV, zg, cKO, ae, d, zb, ff, aF, en, dw, ve, t, bz, zr, zl, eg, zk); #c1(TDRD9) c2(NP_694591) c3 (putative ATP-dependent RNA helicase TORD9) c4(245344, 245345, 245346) c5(245347, 245348, 245349, 245350, 245351, 245352) c6( ) c7(bL, A, cz, j, bK, y); #c1(TDRD9) c2(XP_005267366) c3(putative ATP-dependent RNA helicase TORD9 isoform X6) c4( ) c5(280912, 280913, 280914, 280915, 280916, 280917) c6( ) c7(bL, A, cz, j, bK, y); #c1(TDRD9) c2(XP_011534703) c3(putative ATP-dependent RNA helicase TORD9 isoform X9) c4( ) c5(331193, 331194, 331195, 331196, 331197, 331198) c6( ) c7(bL, A, cz, j, bK, y); #c1(TDRKH) c2(NP_001077433) c3(tudor and KH domain-containing protein isoform b) c4(37868) c5(37869, 37870, 37871, 37872, 37873, 37874) c6( ) c7(iU); #c1(TDRKH) c2(XP_005244913) c3(tudor and KH domain-containing protein isoform X2) c4( ) c5(266161, 266162, 266163, 266164, 266165) c6( ) c7(iU); #c1(TDRKH) c2(XP_011507405) c3(tudor and KH domain-containing protein isoform X1) c4(297286, 297287, 297288, 297289) c5(297290, 297291, 297292, 297293, 297294, 297295, 297296, 297297, 297298, 297299, 297300, 297301, 297302, 297303, 297304, 297305, 297306, 297307, 297308, 297309, 297310, 297311, 297312, 297313, 297314, 297315, 297316, 297317, 297318, 297319, 297320) c6( ) c7(iU); #c1(TDRP) c2(NP_001243042) c3(testis development-related protein isoform 2) c4(89656) c5(89657, 89658, 89659, 89660, 89661, 89662, 89663, 89664, 89665, 89666) c6( ) c7(cm, ac); #c1(TDRP) c2(XP_006716295) c3(testis development-related protein isoform X1) c4(290219) c5(290220, 290221, 290222, 290223, 290224, 290225) c6( ) c7(cm, ac); #c1(TDRP) c2(XP_011533027) c3(testis development-related protein isoform X10) c4( ) c5(329239) c6( ) c7(cm, ac); #c1(TEAD1) c2(NP_068780) c3(transcriptional enhancer factor TEF-1) c4(210502, 210503, 210504, 210505) c5(210506, 210507, 210508, 210509, 210510, 210511, 210512, 210513, 210514, 210515, 210516, 210517, 210518, 210519, 210520, 210521, 210522, 210523, 210524, 210525, 210526, 210527, 210528, 210529, 210530, 210531, 210532, 210533, 210534, 210535, 210536, 210537, 210538, 210539, 210540) c6( ) c7(T, wu, aw, fd, qN, eA, ad, aE, cfd, aT, h, aYf, aUm, K, wm, nJ, cKP, d, Ar, vT, aW, Fm, Y, Lf, Lh, bu, t, bz, qa, ck, VJ, ac, c, eM, U, m, B); #c1(TEAD2) c2(NP_001243587) c3 (transcriptional enhancer factor TEF-4 isoform 1) c4(91107) c5(91108, 91109, 91110, 91111, 91112, 91113, 91114, 91115, 91116, 91117) c6( ) c7(kl); #c1(TEAD2) c2(NP_001243590) c3(transcriptional enhancer factor TEF-4 isoform 2) c4(91118) c5(91119, 91120, 91121, 91122, 91123, 91124, 91125, 91126, 91127, 91128) c6( ) c7(kl); #c1(TEAD2) c2(NP_001243591) c3 (transcriptional enhancer factor TEF-4 isoform 4) c4( ) c5(91129, 91130, 91131, 91132, 91133) c6( ) c7(kl); #c1(TEAD2) c2(NP_003589) c3(transcriptional enhancer factor TEF-4 isoform 3) c4(139393) c5(139394, 139395, 139396, 139397, 139398, 139399, 139400) c6( ) c7(kl); #c1(TEAD2) c2(XP_011525704) c3(transcriptional enhancer factor TEF-4 isoform X3) c4(320121) c5(320122) c6( ) c7(kl); #c1(TEAD3) c2(NP_003205) c3(transcriptional enhancer factor TEF-5) c4(136266, 136267, 136268) c5(136269, 136270, 136271, 136272, 136273, 136274, 136275, 136276, 136277, 136278, 136279, 136280, 136281) c6( ) c7(ac); #c1(TEAD4) c2(NP_003204) c3(transcriptional enhancer factor TEF-3 isoform 1) c4(136242, 136243, 136244, 136245, 136246) c5(136247, 136248, 136249, 136250, 136251, 136252, 136253, 136254, 136255, 136256, 136257, 136258, 136259, 136260, 136261, 136262, 136263, 136264, 136265) c6( ) c7(f, vt, aF, aA); #c1(TEAD4) c2(NP_958849) c3(transcriptional enhancer factor TEF-3 isoform 2) c4(262280) c5(262281, 262282, 262283, 262284, 262285, 262286, 262287, 262288, 262289, 262290, 262291, 262292) c6( ) c7(f, vt, aF, aA); #c1(TEAD4) c2(NP_958851) c3 (transcriptional enhancer factor TEF-3 isoform 3) c4( ) c5(262293, 262294, 262295, 262296, 262297) c6( ) c7(f, vt, aF, aA); #c1(TEC) c2(XP_011512038) c3(tyrosine-protein kinase Tec isoform X1) c4(302693) c5(302694, 302695, 302696, 302697, 302698, 302699) c6( ) c7(hM, kh, aNA, cF, aW, aT, Vv, h, fe, bw, fr, ci, cm, pd, B, br); #c1(TECPR2) c2(NP_001166102) c3(tectonin beta-propeller repeat-containing protein 2 isoform 2) c4(68151, 68152) c5(68153, 68154, 68155, 68156, 68157, 68158) c6( ) c7(cKQ, aV, U); #c1(TECPR2) c2(NP_055659) c3(tectonin beta-propeller repeat-containing protein 2 isoform 1) c4(182567) c5(182568, 182569, 182570, 182571, 182572, 182573, 182574, 182575, 182576, 182577, 182578, 182579) c6( ) c7(cKQ, aV, U); #c1(TECR) c2(NP_612510) c3(very-long-chain endyl-CoA reductase) c4(236676) c5(236677, 236678, 236679, 236680, 236681, 236682) c6( ) c7(sb, T, gZ, nA, be, cKR); #c1(TECR) c2(XP_006723010) c3(very-long-chain enoyl-CoA reductase isoform X3) c4(294980) c5(294981, 294982, 294983, 294984, 294985, 294986) c6( ) c7(sb, T, gZ, nA, be, cKR); #c1(TECRL) c2(NP_001010874) c3(trans-2,3-enoyl-CoA reductase-like) c4(23891) c5(23892, 23893, 23894, 23895, 23896, 23897) c6( ) c7(If); #c1(TECTA) c2(NP_005413) c3(alpha-tectorin precursor) c4(155849) c5(155850, 155851, 155852, 155853, 155854, 155855, 155856, 155857, 155858, 155859) c6( ) c7(cKT, Bh, mG, cKS, cKU); #c1(TEF) c2(NP_001138870) c3(thyrotroph embryonic factor isoform 2) c4(57971, 57972) c5(57973, 57974, 57975, 57976, 57977, 57978, 57979, 57980, 57981, 57982, 57983, 57984, 57985, 57986, 57987, 57988) c6( ) c7(ckM, bkh, bO); #c1(TEF) c2(NP_003207) c3(thyrotroph embryonic factor isoform 1) c4(136282) c5(136283, 136284, 136285, 136286, 136287, 136288, 136289) c6( ) c7(ckM, bkh, bO); #c1(TEFM) c2(NP_078959) c3(transcription elongation factor, mitochondrial) c4(216267, 216268) c5(216269, 216270, 216271, 216272, 216273, 216274, 216275, 216276, 216277, 216278, 216279, 216280, 216281, 216282, 216283, 216284) c6( ) c7(nX, xb, Az, xt); #c1(TEK) c2(NP_000450) c3(angiopoietin-1 receptor isoform 1 precursor) c4(9133) c5(9134, 9135, 9136, 9137, 9138, 9139, 9140, 9141, 9142, 9143) c6( ) c7(ID, T, aC, ea, ci, Vp, lv, Bc, pX, K, eL, ly, eG, aW, Ls, as, bw, uC, cKW, eM, iA, bDr, nB, iT, mx, m, ym, sw, aw, n, aj, oY, fr, Cd, jw, ai, eW, kl, h, dl, cY, cKX, fe, op, lx, dB, Vy, E, ER, bD, B, bL, fM, dA, jp, aA, fF, j, C, bDq, il, Cb, q, eA, eJ, Yn, aT, cy, dz, dl, eu, il, Y, r, t, cKV, ck, nM, ac, wO, dh, bt, df, ad, wD, Jm, dP, hd, iY, ji, fb, pq, lR, et, cr, aF, Fq, qe, bE, uR, lI, lC, y); #c1(TEK) c2(NP_001277006) c3 (angiopoietin-1 receptor isoform 2 precursor) c4(108968) c5(108969, 108970, 108971, 108972, 108973, 108974) c6( ) c7(ID, T, aC, ea, ci, Vp, lv, Bc, pX, K, eL, ly, eG, aW, Ls, as, bw, uC, cKW, eM, iA, bDr, nB, iT, mx, m, ym, sw, aw, n, aj, oY, fr, Cd, jw, ai, eW, kl, h, dL, cY, cKX, fe, op, lx, dB, Vy, E, ER, bD, B, bL, fM, dA, jp, aA, fF, j, C, bDq, il, Cb, q, eA, eJ, Yn, aT, cy, dz, dl, eu, il, Y, r, t, cKV, ck, nM, ac, wl, dh, bt, df, ad, wD, Jm, dP, hd, iY, ji, fb, pq, lR, et, cr, aF, Fq, qe, bE, uR, lI, lC, y); #c1(TEK) c2(NP_001277007) c3(angiopoietin-1 receptor isoform 3 precursor) c4(108975) c5( ) c6( ) c7(ID, T, aC, ea, ci, Vp, lv, Bc, pX, K, eL, ly, eG, aW, Ls, as, bw, uC, cKW, eM, iA, bDr, nB, iT, mx, m, ym, sw, aw, n, aj, oY, fr, Cd, jw, ai, eW, kl, h, dL, cY, CKX, fe, op, lx, dB, Vy, E, ER, bD, B, bL, fM, dA, jp, aA, fF, j, C, bDq, il, Cb, q, eA, eJ, Yn, aT, cy, dz, dl, eu, il, Y, r, t, cKV, ck, nM, ac, wl, dh, bt, df, ad, wD, Jm, dP, hd, iY, ji, fb, pq, lR, et, cr, af, Fq, qe, bE, uR, lI, lC, y); #c1(TEK) c2(XP_005251620) c3(angiopoietin-1 receptor isoform X2) c4(271016) c5(271017, 271018, 271019, 271020, 271021, 271022, 271023, 271024, 271025, 271026, 271027, 271028) c6( ) c7(ID, T, aC, ea, ci, Vp, lv, Bc, pX, K, eL, ly, eG, aW, Ls, as, bw, uC, cKW, eM, iA, bDr, nB, iT, mx, m, ym, sw, aw, n, aj, oY, fr, Cd, jw, ai, eW, kl, h, dl, cY, cKX, fe, op, lx, dB, Vy, E, ER, bD, B, bL, fM, dA, jp, aA, fF, j, C, bDq, il, Cb, q, eA, eJ, Yn, aT, cy, dz, dl, eu, il, Y, r, t, cKV, ck, nM, ac, wl, dh, bt, df, ad, wD, Jm, dP, hd, iY, ji, fb, pq, lR, et, cr, aF, Fq, qe, bE, uR, lI, lC, y); #c1(TEKT1) c2(NP_444515) c3(tektin-1) c4(230368, 230369) c5(230370, 230371, 230372, 230373, 230374, 230375, 230376, 230377, 230378, 230379, 230380, 230381) c6( ) c7(hD); #c1(TEKT5) c2(NP_653275) c3(tektin-5) c4(239471) c5(239472, 239473, 239474, 239475, 239476, 239477) c6( ) c7(bB, bE, T, bu); #c1(TELO2) c2(NP_057195) c3(telomere length regulation protein TEL2 homolog) c4(189428, 189429) c5(189430, 189431, 189432, 189433, 189434, 189435, 189436, 189437, 189438, 189439, 189440, 189441) c6( ) c7(ck, T, os, jh, aA, fd, dz, oZ, aF, nA, y, it); #c1(TENM1) c2(NP_055068) c3(teneurin-1 isoform 3) c4(178729) c5(178730, 178731, 178732, 178733, 178734, 178735, 178736, 178737) c6(178738, 178739) c7(T, kj, bE, r, bB, lQ, q); #c1(TENM1) c2(XP_011529536) c3(teneurin-1 isoform X1) c4(325054) c5(325055, 325056, 325057, 325058, 325059, 325060, 325061, 325062, 325063) c6(325064, 325065) c7(T, kj, bE, r, bB, lQ, q); #c1(TENM2) c2(NP_001116151) c3(teneurin-2) c4(43671, 43672, 43673) c5(43674, 43675, 43676, 43677, 43678, 43679, 43680, 43681, 43682, 43683, 43684, 43685, 43686, 43687, 43688, 43689, 43690, 43691) c6( ) c7(IB, cm, T, cd); #c1(TENM2) c2(XP_011532906) c3 (teneurin-2 isoform X3) c4(329073, 329074, 329075) c5( ) c6( ) c7(IB, cm, T, cd); #c1(TENM3) c2(NP_001073946) c3(teneurin-3) c4(36649, 36650) c5(36651, 36652, 36653, 36654, 36655, 36656, 36657, 36658, 36659, 36660, 36661, 36662, 36663, 36664, 36665, 36666) c6( ) c7(cKY, T, kj, aW, aw, aT, r, n, cu, q); #c1(TENM4) c2(NP_001092286) c3(teneurin-4) c4(39536) c5(39537, 39538, 39539, 39540, 39541) c6( ) c7(bL, bM, ac, af, e, oe, EQ, aT, cz, aj, dy, bK, fq, cd); #c1(TENM4) c2(XP_011543227) c3(teneurin-4 isoform X1) c4(342024) c5(342025) c6( ) c7(bL, bM, ac, af, e, De, EQ, aT, cz, aj, dy, bk, fq, cd); #c1(TENM4) c2(XP_011543231) c3(teneurin-4 isoform X4) c4( ) c5(342026) c6( ) c7(bL, bM, ac, af, e, oe, EQ, aT, cz, aj, dy, bK, fq, cd); #c1(TEP1) c2(XP_005268084) c3(telomerase protein component 1 isoform X1) c4(281486, 281487, 281488, 281489) c5(281490, 281491, 281492, 281493, 281494, 281495, 281496, 281497, 281498, 281499, 281500, 281501) c6( ) c7(bL, Mb, T, e, fr, ci, eA, YD, ad, av, M, aT, dV, bj, dL, d, CN, aW, ff, hL, en, t, bz, ac, iA, nB, at, m, B); #c1(TEP1) c2(XP_011535412) c3(telomerase protein component 1 isoform X2) c4( ) c5(332436, 332437, 332438, 332439, 332440, 332441) c6( ) c7(bL, Mb, T, e, fr, ci, eA, YD, ad, av, M, aT, dV, bj, dL, d, CN, aW, ff, hL, en, t, bz, ac, iA, nB, at, m, B); #c1(TEP1) c2(XP_011535413) c3(telomerase protein component 1 isoform X3) c4( ) c5(332442, 332443, 332444, 332445, 332446, 332447) c6( ) c7(bL, Mb, T, e, fr, ci, eA, YD, ad, av, M, aT, dV, bj, dL, d, CN, aW, ff, hL, en, t, bz, ac, iA, nB, at, m, B); #c1(TEPP) c2(NP_950247) c3(testis, prostate and placenta-expressed protein isoform 1 precursor) c4(260920) c5(260921, 260922, 260923, 260924, 260925, 260926, 260927, 260928) c6(260929, 260930) c7(fe); #c1(TEPP) c2(NP_955535) c3(testis, prostate and placenta-expressed protein isoform 2 precursor) c4(261899) c5(261900, 261901, 261902, 261903, 261904, 261905, 261906, 261907) c6(261908, 261909) c7(fe); #c1(TERF1) c2(NP_003209) c3(telomeric repeat-binding factor 1 isoform 2) c4(136373) c5(136374, 136375, 136376, 136377, 136378, 136379, 136380) c6( ) c7(bL, T, yk, e, WL, aA, cz, fd, cQ, bRy, ci, hF, eA, dP, av, x, auf, kH, aT, aYf, dL, K, hu, ae, pi, uQ, sb, je, iU, aW, Y, xs, uY, lo, aq, af, La, as, cl, Hu, bw, bz, ck, iM, ac, eM, dB, hL, cj, at, dW, oX, k, bC, bt, kS, B, vi); #c1(TERF1) c2(NP_059523) c3(telomeric repeat-binding factor 1 isoform 1) c4(193378) c5(193379, 193380, 193381, 193382, 193383, 193384) c6( ) c7(bL, T, yk, e, WL, aA, cz, fd, cl, bRy, ci, hF, eA, dP, av, x, auf, kH, aT, aYf, dL, K, hu, ae, pi, uQ, sb, je, iU, aW, Y, xs, uY, lo, aq, aF, La, as, cl, Hu, bw, bz, ck, iM, ac, eM, dB, hL, cj, at, dW, oX, k, bC, bt, kS, B, vi); #c1(TERF1) c2(XP_011515884) c3(telomeric repeat-binding factor 1 isoform X3) c4(308898) c5(308899, 308900, 308901, 308902, 308903) c6( ) c7(bL, T, yk, e, WL, aA, cz, fd, cQ, bRy, ci, hF, eA, dP, av, x, auf, kH, aT, aYf, dL, K, hu, ae, pi, uQ, sb, je, iU, aW, Y, xs, uY, lo, aq, aF, La, as, cl, Hu, bw, bz, ck, iM, ac, eM, dB, hL, cj, at, dW, oX, k, bC, bt, kS, B, vi); #c1(TERF2) c2(NP_005643) c3(telomeric repeat-binding factor 2) c4(158220) c5(158221, 158222, 158223, 158224, 158225, 158226) c6( ) c7(bL, T, os, yk, e, jh, aA, bB, tc, oZ, fd, ci, hF, cd, q, ad, auf, MR, KH, aT, h, aYf, dL, K, ae, eL, pl, je, aW, Y, ff, r, fe, aF, en, cl, pl, cm, iV, bz, ck, iM, ac, eM, bE, hk, hL, at, dW, WL, KS, B); #c1(TERF2IP) c2(NP_061848) c3 (telomeric repeat-binding factor 2-interacting protein 1) c4(200764) c5(200765, 200766, 200767, 200768, 200769, 200770) c6( ) c7(bL, sc, T, byb, e, aA, wV, bS, v, ct, ci, ad, aE, dV, aT, bk, K, ae, d, aW, Y, ff, fe, aF, en, pm, Z, t, cm, bz, ik, arJ, nM, W, nb, nB, ac, bE, pc, eM, cj, mf, m, y, bt, B); #c1(TERT) c2(NP_001180305) c3(telomerase reverse transcriptase isoform 2) c4(73790, 73791) c5(73792, 73793, 73794, 73795, 73796, 73797, 73798, 73799, 73800, 73801, 73802, 73803, 73804, 73805, 73806, 73807) c6( ) c7(bM, T, aC, fR, pf, ea, if, e, al, fd, cla, bB, ci, aon, ip, x, Vp, afr, bv, aE, bW, pX, ja, iv, K, hu, aJu, eL, De, aa, DA, aAu, aW, az, hl, ml, cKZ, g, as, pl, eF, cm, QA, bw, iP, yd, qJ, eM, nB, aWZ, wE, bTq, clc, cj, dW, k, m, gn, bsS, pw, QS, sc, dD, ht, NT, n, fr, qN, ee, eW, br, Bl, ft, jz, amJ, zf, cu, lG, aux, hn, dL, h, pi, pl, CN, fe, aq, kp, Hu, cM, Ld, Zj, Wt, op, YE, bP, dB, rW, hk, mg, hL, at, aiS, YH, WL, it, B, fM, dA, AS, fG, aYJ, yk, jx, aA, sM, hy, j, Qo, lu, q, er, eA, eJ, aHu, auf, lg, nv, nX, afQ, Vr, aT, jj, ae, sb, jf, s, Ar, nO, oV, Y, Lf, il, r, en, hf, bu, t, Px, cl, iK, ck, sS, nM, Dy, ac, iM, QG, Cz, nG, np, dy, oX, bt, vi, Gl, kh, id, aD, iW, jh, oH, bRy, eq, ad, afb, hX, dP, av, hd, Db, lC, oW, clb, fb, cjU, d, je, Gv, ff, xs, eH, Ej, aF, iV, bz, LB, es, gk, bF, bE, Lj, cLd, ajw, Lb, bC, hE, kS, iu, y, cjV); #c1(TERT) c2(NP_937983) c3(telomerase reverse transcriptase isoform 1) c4(259450) c5(259451, 259452, 259453, 259454, 259455, 259456) c6( ) c7(bM, T, aC, fR, pf, ea, if, e, al, fd, cLa, bB, ci, aon, ip, x, Vp, afr, bv, aE, bW, pX, ja, iv, K, hu, aJu, eL, de, aa, DA, aAu, aW, az, hl, ml, cKZ, g, as, pl, eF, cm, QA, bw, iP, yd, qJ, eM, nB, aWZ, wE, bTq, clc, cj, dW, k, m, gn, bsS, pw, QS, sc, dD, ht, NT, n, fr, qN, ee, eW, br, Bl, ft, jz, amJ, zf, cU, lG, aux, hn, dL, h, pi, pl, CN, fe, aq, kp, Hu, CM, Ld, Zj, Wt, op, YE, bP, dB, rW, hk, mg, hL, at, aiS, YH, WL, it, B, fM, dA, AS, fG, aYJ, yk, jx, aA, sM, hy, j, Qo, lu, q, er, eA, eJ, aHu, auf, lg, nv, nX, afQ, Vr, aT, jj, ae, sb, jf, s, dr, nO, oV, Y, Lf, iU, r, en, hf, bu, t, Px, cl, ik, ck, sS, nM, Dy, ac, iM, QG, Cz, nG, np, dy, oX, bt, vi, Gl, kh, id, aD, iW, jh, oH, bRy, eq, ad, afb, hX, dP, av, hd, Db, lC, oW, clb, fb, cjU, d, je, Gv, ff, xs, eH, Ej, aF, iV, bz, LB, es, gk, bF, bE, Lj, cLd, ajw, Lb, bC, hE, KS, iu, y, cjV); #c1(TES) c2(NP_056456) c3(testin isoform 1) c4(187616, 187617, 187618) c5(187619, 187620, 187621, 187622, 187623, 187624, 187625, 187626, 187627, 187628, 187629, 187630) c6( ) c7(ck, ix, T, dD, d, hw, x, aW, ea, jh, aT, aA, aF, j, eq, er, ae, ad); #c1(TES) c2(NP_690042) c3(testin isoform 2) c4( ) c5(244801, 244802, 244803, 244804, 244805, 244806) c6( ) c7(ck, ix, T, dD, d, hw, x, aW, ea, jh, aT, aA, aF, j, eq, er, ae, ad); #c1(TES) c2(XP_005250315) c3(testin isoform X1) c4(270253) c5(270254, 270255, 270256, 270257, 270258, 270259) c6( ) c7(ck, ix, T, dD, d, hw, x, aW, ea, jh, aT, aA, aF, j, eq, er, ae, ad); #c1(TESC) c2(NP_001161797) c3(calcineurin B homologous protein 3 isoform 2) c4(66029) c5(66030, 66031, 66032, 66033, 66034, 66035) c6( ) c7(T, dD, hl, ea, cz, yO, ci, eq, hF, Mo, ft, aT, afV, dL, K, aDZ, Xh, eL, jf, d, aW, Y, ff, Xj, en, bz, qa, dr, ck, es, eM, iA, dB, dy, bt, ik, iu); #c1(TESC) c2(NP_060369) c3(calcineurin B homologous protein 3 isoform 1) c4(195640, 195641) c5(195642, 195643, 195644, 195645, 195646, 195647, 195648, 195649, 195650, 195651, 195652, 195653) c6( ) c7(T, dD, hl, ea, cz, yO, ci, eq, hF, Mo, ft, aT, afV, dL, K, aDZ, Xh, eL, jf, d, aW, Y, ff, Xj, en, bz, qa, dr, ck, es, eM, iA, dB, dy, bt, ik, iu); #c1(TESPA1) c2(NP_001129502) c3(protein TESPA1 isoform 1) c4(51717, 51718, 51719, 51720) c5(51721, 51722, 51723, 51724, 51725, 51726, 51727, 51728, 51729, 51730, 51731, 51732, 51733, 51734, 51735, 51736, 51737, 51738, 51739, 51740, 51741, 51742, 51743, 51744) c6( ) c7(ac); #c1(TESPA1) c2(NP_055611) c3(protein TESPA1 isoform 2) c4( ) c5(182270, 182271, 182272, 182273, 182274) c6( ) c7(ac); #c1(TETI) c2(NP_085128) c3(methylcytosine dioxygenase TETI) c4(219338) c5(219339, 219340, 219341, 219342, 219343, 219344) c6( ) c7(ck, T, af, aW, vA, r, aT, gl, dz, azm, y, bt, B, op); #c1(TETI) c2(XP_011538506) c3(methylcytosine dioxygenase TETI isoform X1) c4( ) c5(336255, 336256, 336257, 336258, 336259, 336260) c6( ) c7(ck, T, af, aW, vA, r, aT, gl, dz, azm, y, bt, B, op); #c1(TETI) c2(XP_011538507) c3(methylcytosine dioxygenase TETI isoform X2) c4(336261) c5( ) c6( ) c7(ck, T, af, aW, vA, r, aT, gl, dz, azm, y, bt, B, op); #c1(TETI) c2(XP_011538509) c3(methylcytosine dioxygenase TETI isoform X4) c4( ) c5(336262) c6( ) c7(ck, T, af, aW, vA, r, aT, gl, dz, azm, y, bt, B, op); #c1(TET2) c2(NP_001120680) c3(methylcytosine dioxygenase TET2 isoform a) c4(44517, 44518) c5(44519, 44520, 44521, 44522, 44523, 44524, 44525, 44526, 44527, 44528, 44529, 44530, 44531, 44532, 44533, 44534, 44535, 44536, 44537, 44538, 44539) c6( ) c7(T, fM, Js, iW, qx, fd, EQ, fZ, bs, n, ol, aeu, oY, qy, aqf, fr, abQ, hF, eA, br, ad, er, x, OB, dn, aT, aU, oW, dz, vw, h, jj, pq, ae, Nr, oP, UT, aix, hG, aW, Y, et, gK, il, pr, Mz, kD, po, gm, jh, pm, hf, cl, aKj, bw, aiv, yR, Nq, yd, ck, Ta, pc, UY, iT, kz, hL, cj, pg, dW, k, bt, Jo, Tc); #c1(TET2) c2(NP_060098) c3(methylcytosine dioxygenase TET2 isoform b) c4(194106) c5(194107, 194108, 194109, 194110, 194111, 194112, 194113, 194114, 194115, 194116, 194117, 194118, 194119, 194120, 194121, 194122) c6( ) c7(T, fM, Js, iW, qx, fd, EQ, fZ, bs, n, ol, aeu, oY, qy, aqf, fr, abQ, hF, eA, br, ad, er, x, oB, dn, aT, aU, oW, dz, vw, h, jj, pq, ae, Nr, oP, UT, aix, hG, aW, Y, et, gK, iU, pr, Mz, kD, po, gm, jh, pm, hf, cl, aKj, bw, aiv, yR, Nq, yd, ck, Ta, pc, UY, iT, kz, hL, cj, pg, dW, k, bt, Jo, Tc); #c1(TET2) c2(XP_006714305) c3(methylcytosine dioxygenase TET2 isoform X2) c4(287683) c5(287684, 287685, 287686, 287687, 287688, 287689) c6( ) c7(T, fM, Js, iW, qx, fd, EQ, fZ, bs, n, ol, aeu, oY, qy, aqf, fr, abQ, hF, eA, br, ad, er, x, oB, dn, aT, aU, oW, dz, vw, h, jj, pq, ae, Nr, oP, UT, aix, hG, aW, Y, et, gK, iU, pr, Mz, kD, po, gm, jh, pm, hf, cl, aKj, bw, aiv, yR, Nq, yd, ck, Ta, pc, UY, iT, kz, hL, cj, pg, dW, k, bt, Jo, Tc); #c1(TET2) c2(XP_011530345) c3(methylcytosine dioxygenase TET2 isoform X3) c4(326204, 326205) c5( ) c6( ) c7(T, fM, Js, iW, qx, fd, EQ, fZ, bs, n, ol, aeu, oY, qy, agf, fr, abQ, hF, eA, br, ad, er, x, oB, dn, aT, aU, oW, dz, vw, h, jj, pq, ae, Nr, oP, UT, aix, hG, aW, Y, et, gK, il, pr, Mz, kD, po, gm, jh, pm, hf, cl, aKj, bw, aiv, yR, Nq, yd, ck, Ta, pc, UY, iT, kz, hL, cj, pg, dW, k, bt, Jo, Tc); #c1(TET3) c2(XP_005264244) c3(methylcytosine dioxygenase TET3 isoform X1) c4(278736) c5( ) c6( ) c7(T, pN, vA, dz, bt, B, op); #c1(TET3) c2(XP_011530988) c3(methylcytosine dioxygenase TET3 isoform X4) c4(326939) c5(326940, 326941, 326942, 326943, 326944, 326945, 326946, 326947, 326948, 326949, 326950, 326951) c6( ) c7(T, pN, vA, dz, bt, B, op); #c1(TET3) c2(XP_011530992) c3(methylcytosine dioxygenase TET3 isoform X7) c4( ) c5(326952, 326953) c6( ) c7(T, pN, vA, dz, bt, B, op); #c1(TEX101) c2(NP_001123483) c3(testis-expressed sequence 101 protein isoform 2 precursor) c4( ) c5(47148, 47149, 47150, 47151, 47152) c6( ) c7(T, sS, ac); #c1(TEX101) c2(NP_113639) c3(testis-expressed sequence 101 protein isoform 1) c4(221850, 221851, 221852) c5(221853, 221854, 221855, 221856, 221857, 221858, 221859, 221860, 221861, 221862, 221863, 221864, 221865, 221866, 221867, 221868, 221869) c6( ) c7(T, sS, ac); #c1(TEX11) c2(NP_001003811) c3(testis-expressed sequence 11 protein isoform 1) c4(20188, 20189) c5(20190, 20191, 20192, 20193, 20194, 20195, 20196, 20197, 20198, 20199, 20200, 20201, 20202, 20203, 20204, 20205) c6( ) c7(vH, ND); #c1(TEX11) c2(NP_112566) c3 (testis-expressed sequence 11 protein isoform 2) c4(221404) c5(221405, 221406, 221407, 221408, 221409, 221410) c6( ) c7(vH, ND); #c1(TEX14) c2(NP_001188386) c3(inactive serine/threonine-protein kinase TEX14 isoform c) c4(79099) c5(79100, 79101, 79102, 79103, 79104, 79105) c6( ) c7(vH, aW, Ld, ND, aT); #c1(TEX14) c2(NP_112562) c3(inactive serine/threonine-protein kinase TEX14 isoform b) c4(221397) c5(221398, 221399, 221400, 221401, 221402, 221403) c6( ) c7(vH, aW, Ld, ND, aT); #c1(TEX14) c2(NP_938207) c3(inactive serine/threonine-protein kinase TEX14 isoform a) c4(259808) c5(259809, 259810, 259811, 259812, 259813, 259814) c6( ) c7(vH, aW, Ld, ND, aT); #c1(TEX15) c2(NP_112561) c3(testis-expressed sequence 15 protein) c4(221396) c5( ) c6( ) c7(vH, ND); #c1(TEX15) c2(XP_011542895) c3(testis-expressed sequence 15 protein isoform X3) c4( ) c5(341489, 341490, 341491, 341492, 341493, 341494) c6( ) c7(vH, ND); #c1(TEX264) c2(NP_001230655) c3(testis-expressed sequence 264 protein isoform 1 precursor) c4(86426, 86427, 86428, 86429, 86430) c5(86431, 86432, 86433, 86434, 86435, 86436, 86437, 86438, 86439, 86440, 86441, 86442, 86443, 86444, 86445, 86446, 86447, 86448, 86449, 86450, 86451, 86452, 86453, 86454, 86455, 86456, 86457, 86458, 86459, 86460, 86461, 86462, 86463, 86464, 86465, 86466, 86467, 86468, 86469, 86470, 86471, 86472, 86473, 86474, 86475, 86476, 86477, 86478, 86479, 86480, 86481, 86482, 86483, 86484, 86485, 86486, 86487, 86488, 86489, 86490, 86491, 86492) c6(86493, 86494, 86495, 86496, 86497, 86498, 86499, 86500, 86501, 86502, 86503, 86504, 86505, 86506, 86507, 86508, 86509, 86510, 86511, 86512) c7(ey); #c1(TEX264) c2(XP_011532106) c3(testis-expressed sequence 264 protein isoform X2) c4(328150, 328151) c5(328152, 328153, 328154, 328155, 328156, 328157, 328158, 328159, 328160, 328161) c6( ) c7(ey); #c1(TEX29) c2(NP_689537) c3(testis-expressed sequence 29 protein isoform 2) c4(243027) c5(243028, 243029, 243030, 243031, 243032, 243033) c6( ) c7(cd, gv, j); #c1(TEX30) c2(NP_001273705) c3(testis-expressed sequence 30 protein isoform 3) c4( ) c5(105678, 105679, 105680, 105681, 105682, 105683, 105684, 105685) c6( ) c7(NT, ci, s); #c1(TEX30) c2(XP_005254154) c3(testis-expressed sequence 30 protein isoform X1) c4(273032, 273033) c5(273034, 273035, 273036, 273037, 273038, 273039, 273040, 273041) c6( ) c7(NT, ci, s); #c1(TEX35) c2(NP_001164193) c3(testis-expressed sequence 35 protein isoform 2) c4(66769) c5(66770, 66771, 66772, 66773, 66774, 66775) c6( ) c7(B); #c1(TEX35) c2(NP_001164194) c3 (testis-expressed sequence 35 protein isoform 3) c4(66776) c5(66777, 66778, 66779, 66780, 66781, 66782, 66783, 66784, 66785, 66786, 66787, 66788, 66789, 66790) c6( ) c7(B); #c1(TEX35) c2(NP_001164195) c3 (testis-expressed sequence 35 protein isoform 4) c4(66791) c5(66792, 66793, 66794, 66795, 66796, 66797, 66798, 66799, 66800, 66801, 66802, 66803) c6( ) c7(B); #c1(TEX35) c2(NP_115502) c3(testis-expressed sequence 35 protein isoform 1) c4(222953, 222954, 222955) c5(222956, 222957, 222958, 222959, 222960, 222961) c6( ) c7(B); #c1(TEX40) c2(NP_001034585) c3(testis-expressed sequence 40 protein) c4(31882) c5(31883, 31884, 31885, 31886, 31887, 31888) c6( ) c7(m); #c1(TF) c2(XP_011511402) c3(serotransferrin isoform X1) c4(302011, 302012) c5(302013, 302014, 302015, 302016, 302017, 302018, 302019, 302020, 302021, 302022, 302023, 302024) c6( ) c7(oO, bL, T, Ve, bM, e, aw, yY, v, ah, ci, zP, gb, ad, kL, av, iu, cLe, dY, yt, oW, aT, yu, cy, jj, bq, an, H, ae, af, eu, VF, mu, hi, zQ, d, s, aW, Y, fc, fQ, yy, aq, fe, Z, bN, ck, ar, W, ac, aNv, dB, nB, WP, eM, U, at, y, KS, B, ao, L); #c1(TFAM) c2(NP_001257711) c3(transcription factor A, mitochondrial isoform 2 precursor) c4(95618) c5(95619, 95620, 95621, 95622, 95623, 95624) c6( ) c7(bL, T, JC, aw, jh, aj, aNG, q, J, aoz, cd, cc, MR, hb, oj, aT, lG, vd, K, an, awL, af, ls, bux, aW, fc, r, awJ, t, kC, bz, hJ, Hv, akl, ac, c, Qu, tV, hR, at, acJ, m, ei, gT, cB, p); #c1(TFAM) c2(NP_003192) c3(transcription factor A, mitochondrial isoform 1 precursor) c4(136140, 136141) c5(136142, 136143, 136144, 136145, 136146, 136147, 136148, 136149, 136150, 136151, 136152, 136153) c6( ) c7(bL, T, JC, aw, jh, aj, aNG, q, J, aoz, cd, cc, MR, hb, oj, aT, lG, vd, K, an, awL, af, ls, bux, aW, fc, r, awJ, t, kC, bz, hJ, Hv, akQ, ac, c, Qu, tV, hR, at, acJ, m, ei, gT, cB, p); #c1(TFAM) c2(XP_011538423) c3(transcription factor A, mitochondrial isoform X2) c4( ) c5(336106) c6( ) c7(bL, T, JC, aw, jh, aj, aNG, q, J, aoz, cd, cc, MR, hb, oj, aT, lG, vd, K, an, awL, af, ls, bux, aW, fc, r, awJ, t, kC, bz, hJ, Hv, akQ, ac, c, Qu, tV, hR, at, acJ, m, ei, gT, cB, p); #c1(TFAP2A) c2(NP_001027451) c3(transcription factor AP-2-alpha isoform b) c4(30134) c5(30135, 30136, 30137, 30138, 30139, 30140) c6( ) c7(T, bB, Fb, fr, ci, Xt, q, ad, eA, cc, yQ, Yc, aE, oW, aT, Nc, K, nJ, ae, cp, bVo, li, iU, Jl, aW, Y, ff, Az, r, dN, kp, en, mo, bVp, t, g, as, iK, vU, bF, Dy, bP, lp, bE, hR, dy, m, bt, X); #c1(TFAP2A) c2(NP_001035890) c3(transcription factor AP-2-alpha isoform c) c4(33332) c5(33333, 33334, 33335, 33336, 33337, 33338) c6( ) c7(T, bB, Fb, fr, ci, Xt, q, ad, eA, cc, yQ, Yc, aE, oW, aT, Nc, K, nJ, ae, cp, bVo, li, iU, Jl, aW, Y, ff, Az, r, dN, kp, en, mo, bVp, t, g, as, ik, vU, bF, Dy, bP, lp, bE, hR, dy, m, bt, X); #c1(TFAP2A) c2(NP_003211) c3(transcription factor AP-2-alpha isoform a) c4(136381, 136382, 136383) c5(136384, 136385, 136386, 136387, 136388, 136389, 136390, 136391, 136392, 136393, 136394, 136395, 136396, 136397, 136398, 136399, 136400, 136401, 136402, 136403, 136404, 136405) c6( ) c7(T, bB, Fb, fr, ci, Xt, q, ad, eA, cc, yQ, Yc, aE, oW, aT, Nc, K, nJ, ae, cp, bVo, li, iU, Jl, aW, Y, ff, Az, r, dN, kp, en, mo, bVp, t, g, as, iK, vU, bF, Dy, bP, lp, bE, hR, dy, m, bt, X); #c1(TFAP2B) c2(NP_003212) c3 (transcription factor AP-2-beta) c4(136406) c5(136407, 136408, 136409, 136410, 136411, 136412, 136413) c6( ) c7(bL, atk, Nt, gf, bS, ct, j, v, fr, av, Eo, aT, bK, clf, aW, Y, Az, Au, g, Z, bz, fq, hq, arB, Dy, eM, bP, ei, Xu, iu); #c1(TFAP2B) c2(XP_011513139) c3 (transcription factor AP-2-beta isoform X2) c4(304961, 304962) c5(304963, 304964, 304965, 304966, 304967, 304968, 304969, 304970, 304971, 304972, 304973, 304974) c6( ) c7(bL, atk, Nt, gf, bS, ct, j, v, fr, av, Eo, aT, bK, clf, aW, Y, Az, Au, g, Z, bz, fq, hq, arB, Dy, eM, bP, ei, Xu, iu); #c1(TFAP2C) c2(NP_003213) c3(transcription factor AP-2 gamma) c4(136414, 136415, 136416) c5(136417, 136418, 136419, 136420, 136421, 136422, 136423, 136424, 136425, 136426, 136427, 136428, 136429, 136430, 136431, 136432, 136433, 136434, 136435) c6( ) c7(Bl, ac, wu, aW, Y, vA, al, aA, ci, K, cl, wm, op); #c1(TFAP4) c2(NP_003214) c3(transcription factor AP-4) c4(136436) c5(136437, 136438, 136439, 136440, 136441, 136442, 136443) c6( ) c7(B, q, r); #c1(TFB1M) c2(NP_057104) c3(dimethyladenosine transferase 1, mitochondrial) c4(188909) c5(188910, 188911, 188912, 188913, 188914, 188915, 188916) c6( ) c7(bL, af, as, an); #c1(TFB1M) c2(XP_011534174) c3 (dimethyladenosine transferase 1, mitochondrial isoform X2) c4( ) c5(330472) c6( ) c7(bL, af, as, an); #c1(TFB2M) c2(NP_071761) c3(dimethyladenosine transferase 2, mitochondrial) c4(211989, 211990) c5(211991, 211992, 211993, 211994, 211995, 211996, 211997) c6( ) c7(af, as, an); #c1(TFB2M) c2(XP_011542550) c3(dimethyladenosine transferase 2, mitochondrial isoform X1) c4( ) c5(341096, 341097, 341098, 341099, 341100, 341101) c6( ) c7(af, as, an); #c1(TFCP2) c2(NP_001166924) c3(alpha-globin transcription factor CP2 isoform 3) c4(68583, 68584) c5(68585, 68586, 68587, 68588, 68589, 68590, 68591, 68592, 68593, 68594, 68595, 68596) c6( ) c7(h, agl, af, U, bO); #c1(TFCP2) c2(NP_005644) c3(alpha-globin transcription factor CP2 isoform 1) c4(158227) c5(158228, 158229, 158230, 158231, 158232, 158233, 158234) c6( ) c7(h, agl, af, U, 60); #c1(TFDP1) c2(XP_005268384) c3(transcription factor Dp-1 isoform X1) c4(281769, 281770) c5(281771, 281772, 281773, 281774, 281775, 281776) c6( ) c7(bF, av, af, eM, aW, iW, ff, aT, en, t, bz, B, L); #c1(TFDP1) c2(XP_005268385) c3(transcription factor Dp-1 isoform X2) c4(281777) c5( ) c6( ) c7(bF, av, af, eM, aW, iW, ff, aT, en, t, bz, B, L); #c1(TFDP1) c2(XP_005268387) c3(transcription factor Dp-1 isoform X5) c4( ) c5(281778, 281779, 281780, 281781, 281782, 281783, 281784, 281785, 281786, 281787, 281788, 281789) c6( ) c7(bF, av, af, eM, aW, iW, ff, al, en, t, bz, B, L); #c1(TFDP1) c2(XP_005268388) c3(transcription factor Dp-1 isoform X6) c4(281790) c5(281791, 281792, 281793, 281794, 281795, 281796) c6( ) c7(bF, av, af, eM, aW, iW, ff, aT, en, t, bz, B, L); #c1(TFDP1) c2(XP_011535822) c3(transcription factor Dp-1 isoform X4) c4(333020) c5(333021, 333022, 333023, 333024, 333025, 333026) c6( ) c7(bF, av, af, eM, aW, iW, ff, al, en, t, bz, B, L); #c1(TFDP2) c2(NP_001171609) c3(transcription factor Dp-2 isoform 1) c4(70783, 70784, 70785) c5(70786, 70787, 70788, 70789, 70790, 70791, 70792, 70793, 70794, 70795, 70796, 70797, 70798, 70799, 70800, 70801) c6( ) c7(bP, eR, Db, if, r, dy, q, qN, t, B); #c1(TFDP2) c2(NP_001171610) c3(transcription factor Dp-2 isoform 2) c4(70802, 70803, 70804, 70805) c5(70806, 70807, 70808, 70809, 70810, 70811, 70812, 70813, 70814, 70815, 70816, 70817, 70818, 70819, 70820, 70821, 70822, 70823) c6( ) c7(bP, eR, Db, if, r, dy, q, qN, t, B); #c1(TFDP2) c2(NP_001171611) c3(transcription factor Dp-2 isoform 3) c4(70824) c5(70825, 70826, 70827, 70828, 70829, 70830) c6( ) c7(bP, eR, Db, if, r, dy, q, qN, t, B); #c1(TFDP2) c2(NP_001171612) c3(transcription factor Dp-2 isoform 4) c4(70831) c5(70832, 70833, 70834, 70835, 70836, 70837, 70838) c6( ) c7(bP, eR, Db, if, r, dy, q, qN, t, B); #c1(TFDP2) c2(NP_001171613) c3 (transcription factor Dp-2 isoform 5) c4( ) c5(70839, 70840, 70841, 70842, 70843) c6( ) c7(bP, eR, Db, if, r, dy, q, qN, t, B); #c1(TFDP2) c2(XP_005247788) c3(transcription factor Dp-2 isoform X1) c4(267989, 267990, 267991) c5(267992, 267993, 267994, 267995) c6( ) c7(bP, eR, Db, if, r, dy, q, qN, t, B); #c1(TFDP2) c2(XP_011511409) c3 (transcription factor Dp-2 isoform X4) c4( ) c5(302025, 302026, 302027, 302028, 302029, 302030, 302031, 302032, 302033, 302034, 302035, 302036, 302037, 302038, 302039, 302040, 302041, 302042, 302043, 302044, 302045, 302046, 302047, 302048) c6( ) c7(bP, eR, Db, if, r, dy, q, qN, t, B); #c1(TFDP3) c2(NP_057605) c3(transcription factor Dp family member 3) c4(191707) c5(191708, 191709, 191710, 191711, 191712, 191713) c6( ) c7(aq, B, bu); #c1(TFE3) c2(NP_006512) c3(transcription factor E3 isoform 1) c4(165920) c5( ) c6( ) c7(T, apr, zs, aHx, jh, aj, dG, fr, ci, rP, er, J, py, bPa, bOZ, aim, azH, kj, dq, BG, g, Vi, cm, adj, es, QG, iA, Vy, go, nw, ei, ym, bOQ); #c1(TFE3) c2(XP_006724612) c3(transcription factor E3 isoform X1) c4( ) c5(296113, 296114, 296115, 296116, 296117, 296118, 296119) c6( ) c7(T, apr, zs, aHx, jh, aj, dG, fr, ci, rP, er, J, py, bPa, bOZ, aim, azH, kj, dq, BG, g, Vi, cm, adj, es, QG, iA, Vy, go, nw, ei, ym, bOQ); #c1(TFEB) c2(NP_001161299) c3(transcription factor EB isoform 2) c4(65564, 65565, 65566, 65567, 65568, 65569, 65570, 65571) c5(65572, 65573, 65574, 65575, 65576, 65577, 65578, 65579, 65580, 65581, 65582, 65583, 65584, 65585, 65586, 65587, 65588, 65589, 65590, 65591, 65592, 65593, 65594, 65595, 65596, 65597, 65598, 65599, 65600, 65601, 65602) c6( ) c7(vU, es, am, d, kj, iA, Y, Vi, hl, Lq, j, ci, Kj, v, ei, H, fr, L); #c1(TFEB) c2(NP_001258872) c3(transcription factor EB isoform 3) c4(96620) c5(96621, 96622, 96623, 96624, 96625, 96626, 96627, 96628, 96629, 96630, 96631, 96632, 96633, 96634, 96635) c6( ) c7(vU, es, am, d, kj, iA, Y, Vi, hl, Lq, j, ci, Kj, v, ei, H, fr, L); #c1(TFEB) c2(XP_006715276) c3(transcription factor EB isoform X1) c4(289442) c5(289443, 289444, 289445, 289446, 289447, 289448, 289449, 289450, 289451, 289452, 289453, 289454, 289455, 289456, 289457, 289458, 289459, 289460, 289461, 289462, 289463, 289464, 289465, 289466, 289467, 289468, 289469, 289470, 289471, 289472, 289473, 289474, 289475, 289476, 289477) c6( ) c7(vU, es, am, d, kj, iA, Y, Vi, hl, Lq, j, ci, Kj, v, ei, H, fr, L); #c1(TFEC) c2(NP_001018068) c3(transcription factor EC isoform b) c4( ) c5(27052, 27053, 27054, 27055, 27056, 27057, 27058, 27059, 27060, 27061) c6( ) c7(ey, kj, eZ); #c1(TFEC) c2(NP_001231512) c3(transcription factor EC isoform c) c4(87277) c5(87278, 87279, 87280, 87281, 87282, 87283) c6( ) c7(ey, kj, eZ); #c1(TFEC) c2(NP_036384) c3(transcription factor EC isoform a) c4( ) c5(173450, 173451, 173452, 173453, 173454) c6( ) c7(ey, kj, eZ); #c1(TFEC) c2(XP_011514270) c3(transcription factor EC isoform X2) c4(306845, 306846) c5(306847, 306848, 306849, 306850, 306851, 306852, 306853, 306854) c6( ) c7(ey, kj, eZ); #c1(TFEC) c2(XP_011514272) c3(transcription factor EC isoform X4) c4(306855, 306856) c5(306857, 306858) c6( ) c7(ey, kj, eZ); #c1(TFF1) c2(NP_003216) c3(trefoil factor 1 precursor) c4(136444) c5(136445, 136446, 136447, 136448, 136449, 136450) c6( ) c7(gV, T, Mb, pf, aD, fG, aA, bB, BH, ci, pz, yU, zP, q, ad, XX, jz, di, aE, nv, rJ, bW, aT, h, yZ, dL, ji, hu, MO, H, ae, af, dl, aa, B, s, hG, NE, aW, oV, XE, MD, XC, ff, fe, kp, hp, aB, en, g, aF, t, iV, tR, bF, EW, fT, ac, bP, bE, dJ, lF, at, dy, im, agT, CW, L); #c1(TFF2) c2(NP_005414) c3(trefoil factor 2 precursor) c4(155860) c5(155861, 155862, 155863, 155864, 155865, 155866) c6( ) c7(bM, T, aC, fR, aD, aA, BH, ci, jz, aE, aT, hu, af, dl, aa, li, Jl, aW, ff, az, kt, fe, aF, en, g, iV, bE, z, X); #c1(TFF3) c2(NP_003217) c3(trefoil factor 3 precursor) c4(136451, 136452) c5(136453, 136454, 136455, 136456, 136457, 136458, 136459, 136460, 136461, 136462, 136463, 136464) c6( ) c7(T, AS, Dh, jx, NT, aA, bB, Wb, BF, ci, q, ad, fU, hd, aE, qg, anV, aT, dL, AN, K, ji, hu, ae, bc, B, s, NE, aW, ff, r, fe, pR, aF, hp, zC, en, cl, iV, bF, nM, nB, bE, hL, abt, E, at, aUS, dy, X); #c1(TFG) c2(NP_001007566) c3(protein TFG isoform 1) c4(22824) c5(22825, 22826, 22827, 22828, 22829, 22830, 22831, 22832, 22833, 22834) c6( ) c7(clg, aNA, kh, d, hd, ql, nB, clh, nM, bT, Y, QG, hl, qy, jh, ey, bu, ae, eA, ad, cp); #c1(TFG) c2(XP_005247123) c3(protein TFG isoform X2) c4(267652, 267653, 267654, 267655, 267656) c5(267657, 267658, 267659, 267660, 267661, 267662, 267663, 267664, 267665, 267666, 267667, 267668, 267669, 267670, 267671, 267672, 267673, 267674, 267675, 267676, 267677, 267678, 267679, 267680, 267681, 267682, 267683, 267684, 267685, 267686) c6( ) c7(clg, aNA, kh, d, hd, ql, nB, clh, nM, bT, Y, QG, hl, qy, jh, ey, bU, ae, eA, ad, cp); #c1(TFIP11) c2(XP_011528381) c3(tuftelin-interacting protein 11 isoform X1) c4(323419, 323420, 323421, 323422, 323423, 323424, 323425) c5(323426, 323427, 323428, 323429, 323430, 323431, 323432, 323433, 323434, 323435, 323436, 323437, 323438, 323439, 323440, 323441, 323442, 323443, 323444, 323445, 323446, 323447, 323448, 323449, 323450, 323451, 323452, 323453, 323454, 323455, 323456, 323457, 323458, 323459, 323460, 323461, 323462, 323463, 323464, 323465, 323466, 323467, 323468, 323469, 323470, 323471, 323472, 323473, 323474, 323475, 323476, 323477, 323478, 323479) c6( ) c7(tN, T, Ta, aE, s, bE, ki, cr, r, bB, fe, ci, eE, he, q); #c1(TFP1) c2(NP_001027452) c3(tissue factor pathway inhibitor isoform b precursor) c4(30141, 30142) c5(30143, 30144, 30145, 30146, 30147, 30148, 30149, 30150, 30151, 30152, 30153, 30154, 30155, 30156, 30157, 30158, 30159, 30160, 30161, 30162, 30163, 30164, 30165, 30166, 30167, 30168, 30169, 30170, 30171, 30172, 30173, 30174, 30175, 30176, 30177, 30178, 30179, 30180) c6( ) c7(oO, bM, T, ea, if, al, eb, bB, ci, aY, aMB, K, wt, eL, hm, bpU, ayT, aW, YG, g, uC, wT, YK, P, dD, aw, ez, fr, qN, h, jy, dl, ey, ux, pl, hi, sq, aq, kT, vb, eQ, B, aJJ, eR, DV, fF, cR, j, q, aT, cy, an, ajU, Y, LO, Pg, r, en, kC, iK, bN, pH, cli, Dy, ac, ha, ml, OR, wV, dG, ah, SY, eq, gb, Fc, iJ, hX, ib, qh, ji, Yj, cW, ff, bz, gk, DT, bE, ag, lO, dX, rH); #c1(TFP1) c2(NP_006278) c3(tissue factor pathway inhibitor isoform a precursor) c4(163504, 163505, 163506, 163507, 163508) c5(163509, 163510, 163511, 163512, 163513, 163514, 163515, 163516, 163517, 163518, 163519, 163520, 163521, 163522, 163523, 163524) c6( ) c7(oO, bM, T, ea, if, al, eb, bB, ci, aY, aMB, K, wt, eL, hm, bpU, ayT, aW, YG, g, uC, wT, YK, P, dD, aw, ez, fr, qN, h, jy, dL, ey, ux, pl, hi, sq, aq, kT, vb, eL, B, aJJ, eR, DV, fF, cR, j, q, aT, cy, an, ajU, Y, LO, Pg, r, en, kC, iK, bN, pH, cli, Dy, ac, ha, ml, oR, wV, dG, ah, SY, eq, gb, Fc, iJ, hX, ib, qh, ji, Yj, cW, ff, bz, gk, DT, bE, ag, lO, dX, rH); #c1(TFPI2) c2(NP_001257933) c3(tissue factor pathway inhibitor 2 isoform 3 precursor) c4(95814) c5(95815, 95816, 95817, 95818, 95819, 95820) c6( ) c7(gh, T, dD, aC, wu, ea, if, e, ht, aA, bB, fd, eq, ci, qN, q, ad, eA, eJ, ip, av, aJO, aE, aT, io, h, K, wm, hu, nJ, ae, af, cp, eG, d, s, vT, aW, Fm, Y, Lf, Lh, r, nY, kp, aF, g, bu, t, cm, bz, fs, qa, ck, VJ, ac, c, eM, dB, bE, aep, U, bko, m, y, B); #c1(TFPI2) c2(NP_006519) c3(tissue factor pathway inhibitor 2 isoform 1 precursor) c4(165954) c5(165955, 165956, 165957, 165958, 165959, 165960) c6( ) c7(gh, T, dD, aC, wu, ea, if, e, ht, aA, bB, fd, eq, ci, qN, q, ad, eA, eJ, ip, av, aJO, aE, aT, io, h, K, wm, hu, nJ, ae, af, cp, eG, d, s, vT, aW, Fm, Y, Lf, Lh, r, nY, kp, aF, g, bu, t, cm, bz, fs, qa, ck, VJ, ac, c, eM, dB, bE, aep, U, bko, m, y, B); #c1(TFPT) c2(NP_037474) c3(TCF3 fusion partner) c4(175724) c5(175725, 175726, 175727, 175728, 175729, 175730) c6( ) c7(IR); #c1(TFR2) c2(NP_001193784) c3(transferrin receptor protein 2 isoform 2) c4(82606) c5(82607, 82608, 82609, 82610, 82611, 82612) c6( ) c7(bGu, T, al, cz, aHv, hF, br, x, F, yw, pN, tT, oW, aT, yu, alX, il, atl, hl, yy, aq, as, bw, ck, W, alW, k, pw, y, bt, B, ao); #c1(TFR2) c2(XP_005250610) c3(transferrin receptor protein 2 isoform X1) c4(270444, 270445) c5(270446, 270447, 270448, 270449, 270450, 270451, 270452, 270453, 270454, 270455, 270456) c6( ) c7(bGu, T, al, cz, aHv, hF, br, x, F, yw, pN, tT, oW, aT, yu, alX, il, atl, hl, yy, aq, as, bw, ck, W, aUW, k, pw, y, bt, B, ao); #c1(TFR2) c2(XP_005250611) c3(transferrin receptor protein 2 isoform X2) c4( ) c5(270457, 270458, 270459, 270460, 270461) c6( ) c7(bGu, T, al, cz, aHv, hF, br, x, F, yw, pN, tT, oW, aT, yu, alX, il, atl, hl, yy, aq, as, bw, ck, W, aUW, k, pw, y, bt, B, ao); #c1(TFRC) c2(NP_003225) c3(transferrin receptor protein 1) c4(136465, 136466) c5(136467, 136468, 136469, 136470, 136471, 136472, 136473, 136474, 136475, 136476, 136477, 136478, 136479, 136480) c6( ) c7(T, ea, if, al, fd, bB, ip, bv, cLl, Bc, rJ, dV, K, pk, eL, yo, aW, as, cm, bw, wT, fT, iT, cj, nw, k, m, abX, pw, de, NU, dD, oY, aj, fr, qN, cd, br, eW, h, dL, ey, bl, cY, aq, dw, aBs, bP, asl, dB, xO, at, it, B, bL, jv, jx, rf, AZ, j, v, q, J, eA, yw, aT, clk, yu, dz, jj, jf, kl, s, iU, iZ, Y, r, en, hf, t, ik, ck, BU, kP, awC, aUW, bt, yv, hl, Ve, qb, aZ, iW, jh, bs, aHv, pl, eq, hF, ad, hX, av, CJ, F, clj, oz, oW, iY, ji, Xb, vj, yx, ff, kD, yy, hp, S, Kp, bz, gk, bE, bC, asz, kS); #c1(TG) c2(NP_003226) c3(thyroglobulin precursor) c4(136481, 136482, 136483) c5(136484, 136485, 136486, 136487, 136488, 136489, 136490, 136491, 136492, 136493, 136494, 136495, 136496, 136497, 136498, 136499, 136500, 136501) c6( ) c7(T, aCh, fR, Dd, bzb, bfx, clo, asM, n, aj, hz, pl, ci, boy, bfr, biO, aYs, mC, J, eW, x, Vp, hd, xy, clm, mP, Db, nv, il, RN, clq, hD, h, Fx, yF, A, lx, s, hG, aus, bxz, eZ, fe, bxF, xv, hp, g, iV, zZ, cM, f, bN, qJ, cDe, aZT, nM, bP, nB, auz, Lj, cDd, hr, Lp, cln, abt, clp, k, fl, od, gT, AN, clr); #c1(TG) c2(XP_005251095) c3(thyroglobulin isoform X2) c4(270804, 270805) c5(270806, 270807, 270808, 270809, 270810, 270811) c6( ) c7(T, aCh, fR, Dd, bzb, bfx, clo, asM, n, aj, hz, pl, ci, boy, bfr, biO, aYs, mC, J, eW, x, Vp, hd, xy, clm, mP, Db, nv, il, RN, clq, hD, h, Fx, yF, A, lx, s, hG, aus, bxz, eZ, fe, bxF, xv, hp, g, iV, zZ, cM, f, bN, qJ, cDe, aZT, nM, bP, nB, auz, Lj, cDd, hr, Lp, cLn, abt, clp, k, fl, od, gT, AN, clr); #c1(TG) c2(XP_005251100) c3(thyroglobulin isoform X5) c4( ) c5(270812, 270813, 270814, 270815, 270816, 270817) c6( ) c7(T, aCh, fR, Dd, bzb, bfx, clo, asM, n, aj, hz, pl, ci, boy, bfr, biO, aYs, mC, J, eW, x, Vp, hd, xy, clm, mP, Db, nv, il, RN, clq, hD, h, Fx, yF, A, lx, s, hG, aus, bxz, eZ, fe, bxF, xv, hp, g, iV, zZ, cM, f, bN, qJ, cDe, aZT, nM, bP, nB, auz, Lj, cDd, hr, Lp, cln, abt, clp, k, fl, od, gT, AN, clr); #c1(TGFA) c2(NP_001093161) c3(protransforming growth factor alpha isoform 2 preproprotein) c4(39897) c5(39898, 39899, 39900, 39901, 39902, 39903, 39904, 39905) c6( ) c7(oO, azs, T, aC, if, al, bB, ci, ip, Na, aE, pX, aeO, K, hu, bky, eL, aa, DA, yo, bjy, aeR, aW, aEl, kt, g, as, eF, zE, uC, cm, Qt, eM, iA, AM, go, oy, NI, uJ, m, iu, aVF, fr, btF, auO, b, no, amJ, gd, h, ey, Gc, MD, fe, atf, aq, iO, hL, pV, it, B, rG, jp, Dh, fG, Lx, jx, aA, cls, hy, v, AO, q, er, eA, lg, nv, BJ, HC, aT, Nc, jj, ae, dl, s, Y, ev, r, en, bu, cl, t, KQ, iK, tR, sS, nM, kP, Dy, ac, bpv, gX, CZ, dy, fD, rA, VB, vi, gx, jh, Hn, jE, ad, hX, av, hd, F, ib, py, Db, hP, nJ, Yi, d, qt, vj, ff, xs, PA, eH, cr, aF, hp, anW, iR, KM, bz, LB, es, apM, zr, bE, uR, azT, aKC, aS, ra, i, lC, KS, X, y); #c1(TGFA) c2(NP_003227) c3(protransforming growth factor alpha isoform 1 preproprotein) c4(136502) c5(136503, 136504, 136505, 136506, 136507, 136508, 136509, 136510) c6( ) c7(oO, azs, T, aC, if, al, bB, ci, ip, Na, aE, pX, aeO, K, hu, bky, eL, aa, DA, yo, bjy, aeR, aW, aEl, kt, g, as, eF, zE, uC, cm, At, eM, iA, AM, go, oy, NI, uJ, m, iu, aVF, fr, btF, auO, b, no, amJ, gd, h, ey, Gc, MD, fe, atf, aq, iO, hL, pV, it, B, rG, jp, Dh, fG, Lx, jx, aA, cls, hy, v, AO, q, er, eA, lg, nv, BJ, HC, aT, Nc, jj, ae, dl, s, Y, ev, r, en, bu, cl, t, KQ, iK, tR, sS, nM, kP, Dy, ac, bpv, gX, CZ, dy, fD, rA, VB, vi, gx, jh, Hn, jE, ad, hX, av, hd, F, ib, py, Db, hP, nJ, Yi, d, qt, vj, ff, xs, PA, eH, cr, aF, hp, anW, iR, KM, bz, LB, es, apM, zr, bE, uR, azT, aKC, aS, ra, i, lC, KS, X, y); #c1(TGFB1) c2(NP_000651) c3(transforming growth factor beta-1 precursor) c4(14170, 14171, 14172) c5(14173, 14174, 14175, 14176, 14177, 14178, 14179, 14180, 14181, 14182, 14183, 14184, 14185, 14186, 14187, 14188, 14189, 14190, 14191, 14192, 14193, 14194, 14195, 14196, 14197, 14198, 14199, 14200, 14201, 14202, 14203, 14204, 14205, 14206, 14207, 14208, 14209, 14210, 14211, 14212, 14213, 14214, 14215, 14216, 14217, 14218, 14219, 14220, 14221, 14222, 14223, 14224, 14225, 14226, 14227, 14228, 14229, 14230, 14231, 14232, 14233, 14234, 14235, 14236, 14237, 14238, 14239, 14240, 14241, 14242, 14243, 14244, 14245, 14246, 14247, 14248, 14249, 14250, 14251, 14252, 14253, 14254, 14255, 14256, 14257, 14258, 14259, 14260, 14261, 14262, 14263, 14264, 14265, 14266, 14267, 14268) c6(14269) c7(T, ea, Av, aza, fd, aoj, ci, bRn, aY, bv, aE, dV, BZ, Ds, hu, zy, hH, aiJ, aa, li, ly, yo, az, nY, uY, zC, ba, fT, Jr, nB, wE, bhl, cj, Zn, cx, k, fy, ht, biB, cQ, aj, cd, br, vM, aSa, Vl, bkP, lG, Gw, ey, aak, on, mR, Ln, anP, aJ, sp, EU, Hz, aRL, ge, fQ, fe, adz, eD, Be, E, DM, aA, cz, atp, AO, q, zn, jj, sX, bA, bf, bg, lC, sV, lo, eS, cl, pb, sk, rR, OW, ac, iM, ig, CP, bt, ao, gV, iW, uG, jN, jh, dG, uu, ad, hX, bRP, yt, iY, bgr, lI, amY, bPM, NP, ff, afi, dq, iV, bz, DF, ag, aDB, azT, aKC, lf, lC, ra, ne, iu, aRD, oO, bM, lD, qo, vS, if, e, bB, cL, ur, bh, sD, Tz, ip, aHR, Na, cfq, ws, fS, bc, eG, up, dH, fc, hl, Qm, eE, eF, cm, bw, qJ, vA, oy, mx, vy, in, bBT, dD, gU, akc, zt, n, oY, uE, jz, eV, DU, A, RT, AQ, lb, po, bxh, vo, bP, CA, hl, at, pg, ut, B, pF, dA, mr, wL, ex, fF, GC, v, Zk, V, MK, clx, sy, ld, aWJ, bau, ae, hM, zH, ev, afq, acB, bu, t, ck, cly, amP, yH, zu, gO, vi, dM, eq, BY, Tg, hd, py, abP, hD, rN, Dj, JK, XI, nJ, sj, d, je, et, rM, KD, Z, arv, bF, D, gk, yE, Ny, dO, wz, gZ, Hs, y, ng, asg, aC, fR, al, CH, axk, gf, IR, Vp, AW, rJ, bW, M, rB, K, clu, wt, dF, eL, HI, axu, asD, mQ, aGQ, aB, g, ve, bwe, uC, iP, ou, aDs, uy, U, uo, dW, Hb, m, NK, aFQ, sl, sw, zX, lo, aw, NT, fN, BH, bdL, pM, asS, ft, KH, Lv, CU, Ha, CN, jT, cM, Ld, Zj, ar, kT, wk, mg, gg, Pr, it, L, fM, fG, De, jx, wo, hz, j, aYW, pd, eA, J, er, XX, lg, hb, nX, Nc, bq, aRR, kl, xR, Y, Pg, r, en, En, wA, iK, sn, c, ald, hc, azk, Da, dy, fD, LS, Gl, sY, kh, am, id, CS, aZ, bgQ, em, uO, bS, ah, Jm, dP, clw, oj, io, aSf, ji, Yi, lU, atA, vj, eH, cr, clt, hp, KM, qa, adA, Nq, RQ, TA, Bv, pu, Lj, X, aCl, cP, ei, hE, vt, EB, G, eb, aTy, Lw, Jx, bOy, il, avA, qM, apj, dl, ass, bQv, hm, DA, zQ, aW, eM, wK, Jp, wB, clv, uJ, abp, R, qR, clz, vl, tp, wh, fr, ct, bd, qN, aPb, ai, eW, amB, cc, aOu, wG, rl, h, dL, pj, hB, SD, WC, Nr, Dg, aq, kp, cFn, mo, dw, op, lx, Wt, vD, lX, hk, bm, gw, uz, hR, aDE, bni, bL, Dw, eR, uv, cR, hy, C, aoC, ady, mj, ga, blC, mP, uT, qp, afQ, aT, bK, zg, af, Uf, s, iU, yL, la, akY, aDm, bQ, f, brm, lY, nM, pZ, fP, aJY, im, Ke, Zl, qb, aD, vr, Fb, jE, aFJ, KL, av, F, ib, vw, vO, uQ, qt, eZ, yy, gl, aF, ek, LB, es, eU, pc, Uj, bE, z, aS, be, adg, cbO, us); #c1(TGFB1) c2(XP_011525544) c3(transforming growth factor beta-1 isoform X1) c4(319975) c5( ) c6( ) c7(T, ea, Av, aza, fd, aoj, ci, bRn, aY, bv, aE, dV, BZ, Ds, hu, zy, hH, aiJ, aa, li, ly, yo, az, nY, uY, zC, ba, fT, Jr, nB, wE, bhl, cj, Zn, cX, k, fy, ht, biB, cQ, aj, cd, br, vM, aSa, Vl, bkP, lG, Gw, ey, aak, on, mR, Ln, anP, aJ, sp, EU, Hz, aRL, ge, fQ, fe, adz, eD, Be, E, DM, aA, cz, atp, AO, q, zn, jj, sX, bA, bf, bg, lC, sV, lo, eS, cl, pb, sk, rR, OW, ac, iM, ig, CP, bt, ao, gV, iW, uG, jN, jh, dG, uu, ad, hX, bRP, yt, iY, bgr, lQ, amY, bPM, NP, ff, afi, dq, iV, bz, DF, ag, aDB, azT, aKC, lf, lC, ra, ne, iu, aRD, oO, bM, lD, qo, vS, if, e, bB, cL, ur, bh, sD, Tz, ip, aHR, Na, cfq, ws, fS, bc, eG, up, dH, fc, hl, Qm, eE, eF, cm, bw, qJ, vA, oy, mx, vy, in, bBT, dD, gU, akc, zt, n, oY, uE, jz, eV, DU, A, RT, AQ, lb, po, bxh, vo, bP, cA, hL, at, pg, ut, B, pF, dA, mr, wL, eX, fF, GC, v, Zk, V, MK, clx, sy, ld, aWJ, bau, ae, hM, zH, ev, afq, acB, bu, t, ck, cly, amP, yH, zu, gO, vi, dM, eq, BY, Tg, hd, py, abP, hD, rN, Dj, JK, XI, nJ, sj, d, je, et, rM, KD, Z, arv, bF, D, gk, yE, Ny, dO, wz, gZ, Hs, y, ng, asg, aC, fR, al, CH, axk, gf, lR, Vp, AW, rJ, bW, M, rB, K, clu, wt, dF, eL, HI, axu, asD, mQ, aGQ, aB, g, ve, bwe, uC, iP, ou, aDs, uy, U, uo, dW, Hb, m, NK, aFQ, sQ, sw, zX, lo, aw, NT, fN, BH, bdL, pM, asS, ft, KH, Lv, CU, Ha, CN, jT, cM, Ld, Zj, ar, kT, wk, mg, gg, Pr, it, L, fM, fG, oe, jx, wo, hz, j, aYW, pd, eA, J, er, XX, lg, hb, nX, Nc, bq, aRR, kl, xR, Y, Pg, r, en, En, wA, iK, sn, c, and, hc, azk, Da, dy, fD, LS, Gl, sY, kh, am, id, CS, aZ, bgQ, em, uO, bS, ah, Jm, dP, clw, oj, io, aSf, ji, Yi, lU, atA, vj, eH, cr, clt, hp, KM, qa, adA, Nq, RQ, TA, Bv, pu, Lj, X, aCl, cP, ei, hE, vt, EB, G, eb, aTy, Lw, Jx, bOy, il, avA, qM, apj, dl, ass, bQv, hm, DA, zQ, aW, eM, wK, Jp, wB, clv, uJ, abp, R, qR, cLz, vl, tp, wh, fr, ct, bd, qN, aPb, ai, eW, amB, cc, aOu, wG, rl, h, dL, pj, hB, SD, WC, Nr, Dg, aq, kp, cFn, mo, dw, op, lx, Wt, vD, lX, hk, bm, gw, uz, hR, aDE, bni, bL, Dw, eR, uv, cR, hy, C, aoC, ady, mj, ga, blC, mP, uT, qp, afQ, aT, bK, zg, af, Uf, s, iU, yL, la, akY, aDm, bQ, f, brm, lY, nM, pZ, fP, aJY, im, Ke, Zl, qb, aD, vr, Fb, jE, aFJ, KL, av, F, ib, vw, vO, uQ, qt, eZ, yy, gl, af, ek, LB, es, eU, pc, Uj, bE, z, aS, be, adg, cbO, us); #c1(TGFB111) c2(NP_001035919) c3(transforming growth factor beta-1-induced transcript 1 protein isoform 1) c4(33390, 33391) c5(33392, 33393, 33394, 33395, 33396, 33397, 33398, 33399, 33400, 33401, 33402) c6(33403, 33404) c7(dP, T, Ln, vH, n, PU, pt, ae, LS, ad); #c1(TGFB111) c2(NP_001158191) c3(transforming growth factor beta-1-induced transcript 1 protein isoform 2) c4( ) c5(63309, 63310, 63311, 63312, 63313, 63314, 63315, 63316, 63317, 63318, 63319, 63320) c6(63321, 63322, 63323, 63324) c7(dP, T, Ln, vH, n, PU, pt, ae, LS, ad); #c1(TGFB2) c2(NP_001129071) c3(transforming growth factor beta-2 isoform 1 precursor) c4(50730) c5(50731, 50732, 50733, 50734, 50735, 50736, 50737) c6( ) c7(bM, T, al, Av, aza, bB, aoj, ci, aY, aZO, xE, aE, ws, dV, Ds, K, cpm, eG, dH, aW, wf, Az, Du, g, eF, cm, eM, mx, bru, m, vF, eg, dt, dD, vl, tp, wh, lo, aw, n, eh, ft, ju, Ha, PX, dw, cLA, Qf, eD, Be, bD, apE, B, Dw, eR, DD, dk, fG, v, eA, uT, DK, aT, bq, rv, ae, jf, Y, An, en, t, sS, c, dy, cfe, Mb, kh, id, aZ, rl, jh, eq, ad, dP, F, LA, ab, lQ, baU, cLC, uQ, d, ff, eH, eZ, rM, hp, iV, atb, cLB, D, Uj, bE, z, aEY, wz, Hs, be, lf, y); #c1(TGFB2) c2(NP_003229) c3(transforming growth factor beta-2 isoform 2 precursor) c4(136511) c5(136512, 136513, 136514, 136515, 136516, 136517, 136518) c6( ) c7(bM, T, al, Av, aza, bB, aoj, ci, aY, aZO, xE, aE, ws, dV, Ds, K, cpm, eG, dH, aW, wF, Az, Du, g, eF, cm, eM, mx, bru, m, vF, eg, dt, dD, vl, tp, wh, lo, aw, n, eh, ft, ju, Ha, PX, dw, cLA, Of, eD, Be, bD, apE, B, Dw, eR, DD, dk, fG, v, eA, uT, DK, aT, bq, rv, ae, jf, Y, An, en, t, sS, c, dy, cfe, Mb, kh, id, aZ, rl, jh, eq, ad, dP, F, LA, ab, lQ, baU, cLC, uQ, d, ff, eH, eZ, rM, hp, iV, atb, cLB, D, Uj, bE, z, aEY, wz, Hs, be, lf, y); #c1(TGFB3) c2(NP_003230) c3(transforming growth factor beta-3 preproprotein) c4(136519, 136520, 136521) c5(136522, 136523, 136524, 136525, 136526, 136527, 136528, 136529, 136530, 136531, 136532, 136533, 136534, 136535, 136536, 136537, 136538, 136539, 136540, 136541, 136542, 136543, 136544, 136545, 136546, 136547, 136548, 136549, 136550, 136551, 136552, 136553, 136554, 136555, 136556, 136557, 136558, 136559, 136560, 136561, 136562, 136563, 136564, 136565, 136566, 136567, 136568, 136569, 136570, 136571, 136572, 136573, 136574, 136575, 136576, 136577, 136578, 136579, 136580, 136581, 136582, 136583, 136584, 136585, 136586, 136587, 136588, 136589, 136590, 136591, 136592, 136593, 136594, 136595, 136596, 136597, 136598, 136599, 136600, 136601, 136602, 136603, 136604, 136605, 136606, 136607, 136608, 136609, 136610, 136611, 136612, 136613, 136614, 136615, 136616, 136617, 136618, 136619, 136620, 136621, 136622, 136623, 136624, 136625) c6(136626, 136627, 136628, 136629, 136630) c7(T, dt, kh, dD, AL, tp, fG, al, rl, aza, cz, n, eb, v, j, ci, cLE, eq, ad, Na, av, ft, F, uT, amJ, nX, ju, aT, Nc, cBf, ae, cpm, sj, uQ, aoa, bf, aW, Y, eZ, r, rM, hp, g, cl, iV, t, bwe, uC, op, D, sS, apM, eM, nB, wK, vA, cLD, bm, dy, m, y, iu); #c1(TGFB1) c2(NP_000349) c3(transforming growth factor-beta-induced protein ig-h3 precursor) c4(7632, 7633, 7634) c5(7635, 7636, 7637, 7638, 7639, 7640, 7641, 7642, 7643, 7644, 7645, 7646, 7647, 7648, 7649, 7650, 7651, 7652) c6( ) c7(Mb, T, dD, aC, id, fG, al, cLl, j, ci, ur, uu, eq, lR, q, ad, eA, av, iu, alu, py, cLH, bFl, dv, ab, ey, H, bXu, ae, aPB, uX, cLK, Lu, cLG, bwk, Y, la, la, cLF, boD, t, bz, pb, cm, adz, kT, gk, cLJ, cLM, dy, m, R, cLL, y, it, B, jr); #c1(TGFBR1) c2(NP_001124388) c3(TGF-beta receptor type-1 isoform 2 precursor) c4(48777, 48778) c5(48779, 48780, 48781, 48782, 48783, 48784, 48785, 48786, 48787, 48788, 48789) c6( ) c7(bM, T, aC, if, e, al, Av, aza, bB, lR, ci, Na, AW, aE, Ds, hH, eL, hm, yo, aiV, aW, hl, g, eF, bw, y, eM, vA, cj, uo, Dt, k, Hb, m, Th, asW, vl, brL, tp, aj, Mi, nA, qN, brK, ai, ft, kl, dL, Ck, fQ, kp, alf, dw, op, hk, bm, at, YH, bD, Pr, B, Dw, dA, yk, aA, cR, cz, wo, Eh, q, nX, yg, aT, bfq, rv, ae, qw, bf, xU, s, Y, yL, r, en, t, ik, pb, amF, ac, dy, bt, iW, atS, jh, ad, av, F, yt, lQ, WW, ff, eH, eZ, aF, si, bz, cLB, es, brG, bE, aS, be, lC, X, us); #c1(TGFBR1) c2(NP_004603) c3(TGF-beta receptor type-1 isoform 1 precursor) c4(148068, 148069, 148070) c5(148071, 148072, 148073, 148074, 148075, 148076, 148077, 148078, 148079, 148080, 148081, 148082, 148083, 148084, 148085, 148086, 148087, 148088, 148089) c6( ) c7(bM, T, aC, if, e, al, Av, aza, bB, lR, ci, Na, AW, aE, Ds, hH, eL, hm, yo, aiV, aW, hl, g, eF, bw, y, eM, vA, cj, uo, Dt, k, Hb, m, Th, asW, vl, brL, tp, aj, Mi, nA, qN, brK, ai, ft, kl, dL, Ck, fQ, kp, alf, dw, op, hk, bm, at, YH, bD, Pr, B, Dw, dA, yk, aA, cR, cz, wo, Eh, q, nX, yg, aT, bfq, rv, ae, qw, bf, xU, s, Y, yL, r, en, t, ik, pb, amF, ac, dy, bt, iW, atS, jh, ad, av, F, yt, IQ, WW, ff, eH, eZ, aF, si, bz, cLB, es, brG, bE, aS, be, lC, X, us); #c1(TGFBR2) c2(NP_001020018) c3(TGF-beta receptor type-2 isoform A precursor) c4(28055) c5(28056, 28057, 28058, 28059, 28060, 28061, 28062) c6( ) c7(oO, bM, T, lD, Ap, aC, e, al, Av, aza, bB, aeu, ur, ci, qj, lR, ip, Na, AW, aE, iv, cLP, K, hu, bc, aiJ, de, ly, aW, Az, nY, uY, Au, g, bw, eM, nB, cj, uo, k, m, Xu, iu, brF, vl, brL, tp, RX, ht, aw, n, Mi, fr, uE, nA, bao, br, Bl, ft, jz, zw, lG, Gw, hB, dx, bhg, fQ, kp, yq, lx, Qy, bP, Qu, cLN, hL, aXv, pg, bm, bD, Pr, B, it, bL, fM, eR, fG, oe, rf, aA, cz, ti, hy, j, Eh, q, eA, cLO, brH, nX, afQ, yg, aT, Tj, ae, qw, bf, s, oV, Y, yL, fO, r, aYp, en, cl, t, ik, pb, amF, ck, cT, sS, ac, DW, dy, im, JO, Gl, iW, atS, rl, jh, uu, ad, BY, av, F, brB, io, ji, baU, WW, BO, ff, xs, eZ, rM, aF, si, iV, bz, ek, RV, brG, bE, aS, be, Gu, lC, X, us); #c1(TGFBR2) c2(NP_003233) c3(TGF-beta receptor type-2 isoform B precursor) c4(136645) c5(136646, 136647, 136648, 136649, 136650, 136651) c6( ) c7(oO, bM, T, lD, Ap, aC, e, al, Av, aza, bB, aeu, ur, ci, qj, lR, ip, Na, AW, aE, iv, cLP, K, hu, bc, aiJ, De, ly, aW, Az, nY, uY, Au, g, bw, eM, nB, cj, uo, k, m, Xu, iu, brF, vl, brL, tp, RX, ht, aw, n, Mi, fr, uE, nA, bao, br, Bl, ft, jz, zw, lG, Gw, hB, dx, bhg, fQ, kp, yq, lx, Qy, bP, Qu, CLN, hL, aXv, pg, bm, bD, Pr, B, it, bL, fM, eR, fG, oe, rf, aA, cz, ti, hy, j, Eh, q, eA, cLO, brH, nX, afQ, yg, aT, Tj, ae, qw, bf, s, oV, Y, yL, fO, r, aYp, en, cl, t, iK, pb, amF, ck, cT, sS, ac, DW, dy, im, JO, Gl, iW, atS, rl, jh, uu, ad, BY, av, F, brB, io, ji, baU, WW, BO, ff, xs, eZ, rM, aF, si, iV, bz, ek, RV, brG, bE, aS, be, Gu, lC, X, us); #c1(TGFBR2) c2(XP_011532345) c3(TGF-beta receptor type-2 isoform X1) c4(328499) c5(328500) c6( ) c7(oO, bM, T, lD, Ap, aC, e, al, Av, aza, bB, aeu, ur, ci, qj, lR, ip, Na, AW, aE, iv, cLP, K, hu, bc, aiJ, De, ly, aW, Az, nY, uY, Au, g, bw, eM, nB, cj, uo, k, m, Xu, iu, brF, vl, brL, tp, RX, ht, aw, n, Mi, fr, uE, nA, bao, br, Bl, ft, jz, zw, lG, Gw, hB, dx, bhg, fQ, kp, yq, lx, Qy, bP, Qu, CLN, hL, aXv, pg, bm, bD, Pr, B, it, bL, fM, eR, fG, oe, rf, aA, cz, ti, hy, j, Eh, q, eA, cLO, brH, nX, afQ, yg, aT, Tj, ae, qw, bf, s, oV, Y, yL, fO, r, aYp, en, cl, t, ik, pb, amF, ck, cT, sS, ac, DW, dy, im, JO, Gl, iW, atS, rl, jh, uu, ad, BY, av, F, brB, io, ji, baU, WW, BO, ff, xs, eL, rM, aF, si, iV, bz, ek, RV, brG, bE, aS, be, Gu, lC, X, us); #c1(TGFBR2) c2(XP_011532346) c3(TGF-beta receptor type-2 isoform X2) c4(328501) c5( ) c6( ) c7(oO, bM, T, ID, Ap, aC, e, al, Av, aza, bB, aeu, ur, ci, qj, lR, ip, Na, AW, aE, iv, cLP, K, hu, bc, aiJ, de, ly, aW, Az, nY, uY, Au, g, bw, eM, nB, cj, uo, k, m, Xu, iu, brf, vl, brL, tp, RX, ht, aw, n, Mi, fr, uE, nA, bao, br, Bl, ft, jz, zw, lG, Gw, hB, dx, bhg, fQ, kp, yq, lx, Qy, bP, Qu, cLN, hl, aXv, pg, bm, bD, Pr, B, it, bL, fM, eR, fG, oe, rf, aA, cz, ti, hy, j, Eh, q, eA, cLO, brH, nX, afQ, yg, aT, Tj, ae, qw, bf, s, oV, Y, yL, fO, r, aYp, en, cl, t, ik, pb, amF, ck, cT, sS, ac, DW, dy, im, JO, Gl, iW, atS, rl, jh, uu, ad, BY, av, F, brB, io, ji, baU, WW, BO, ff, xs, eZ, rM, aF, si, iV, bz, ek, RV, brG, bE, aS, be, Gu, lC, X, us); #c1(TGFBR2) c2(XP_011532347) c3(TGF-beta receptor type-2 isoform X3) c4( ) c5(328502, 328503, 328504, 328505, 328506, 328507, 328508, 328509, 328510, 328511) c6( ) c7(oO, bM, T, lD, Ap, aC, e, al, Av, aza, bB, aeu, ur, ci, qj, lR, ip, Na, AW, aE, iv, CLP, K, hu, bc, aiJ, De, ly, aW, Az, nY, uY, Au, g, bw, eM, nB, cj, uo, k, m, Xu, iu, brF, vl, brL, tp, RX, ht, aw, n, Mi, fr, uE, nA, bao, br, Bl, ft, jz, zw, lG, Gw, hB, dx, bhg, fQ, kp, yq, lx, Qy, bP, Qu, cLN, hl, aXv, pg, bm, bD, Pr, B, it, bL, fM, eR, fG, oe, rf, aA, cz, ti, hy, j, Eh, q, eA, cLO, brH, nX, afQ, yg, aT, Tj, ae, qw, bf, s, oV, Y, yL, fO, r, aYp, en, cl, t, iK, pb, amF, ck, cT, sS, ac, DW, dy, im, JO, Gl, iW, atS, rl, jh, uu, ad, BY, av, F, brB, io, ji, baU, WW, BO, ff, xs, eZ, rM, aF, si, iV, bz, ek, RV, brG, bE, aS, be, Gu, lC, X, us); #c1(TGFBRAP1) c2(NP_001136093) c3(transforming growth factor-beta receptor-associated protein 1) c4(54315, 54316) c5(54317, 54318, 54319, 54320, 54321, 54322, 54323, 54324, 54325, 54326, 54327, 54328, 54329, 54330, 54331, 54332) c6( ) c7(T, F, il, tL, aW, ht, aT, r, K, uE, hu, it, q); #c1(TGIF1) c2(NP_001265611) c3(homeobox protein TGIF1 isoform e) c4( ) c5(98973, 98974, 98975) c6( ) c7(asg, s, bPl, aBU, YX, bPt, FB, aJ, QM, Be, ZW, ff, gl, dL, bl, bd, en, el); #c1(TGIF1) c2(NP_001265613) c3(homeobox protein TGIF1 isoform c) c4(98976) c5(98977, 98978, 98979, 98980, 98981, 98982, 98983, 98984, 98985, 98986, 98987, 98988, 98989, 98990, 98991, 98992, 98993) c6( ) c7(asg, s, bPl, aBU, YX, bPt, FB, aJ, QM, Be, ZW, ff, gl, dl, bl, bd, en, el); #c1(TGIF1) c2(NP_733796) c3(homeobox protein TGIF1 isoform a) c4(247127) c5(247128, 247129, 247130, 247131, 247132, 247133, 247134, 247135, 247136, 247137, 247138) c6( ) c7(asg, s, bPl, aBU, YX, bPt, FB, aJ, QM, Be, ZW, ff, gl, dL, bl, bd, en, el); #c1(TGIF1) c2(NP_775299) c3(homeobox protein TGIF1 isoform b) c4(249938, 249939, 249940, 249941, 249942, 249943, 249944, 249945, 249946) c5(249947, 249948, 249949, 249950, 249951, 249952, 249953, 249954, 249955, 249956) c6( ) c7(asg, s, bPl, aBU, YX, bPt, FB, aJ, QM, Be, ZW, ff, gl, dL, bl, bd, en, el); #c1(TGIF1) c2(XP_011524037) c3(homeobox protein TGIF1 isoform X1) c4( ) c5(318566, 318567, 318568, 318569, 318570, 318571, 318572, 318573, 318574, 318575, 318576, 318577, 318578, 318579, 318580, 318581, 318582, 318583, 318584, 318585, 318586, 318587, 318588, 318589, 318590, 318591, 318592, 318593, 318594, 318595, 318596, 318597, 318598, 318599, 318600, 318601, 318602, 318603, 318604, 318605, 318606, 318607, 318608, 318609, 318610, 318611, 318612, 318613, 318614, 318615, 318616, 318617, 318618, 318619, 318620) c6( ) c7(asg, s, bPl, aBU, YX, bPt, FB, aJ, QM, Be, ZW, ff, gl, dL, bl, bd, en, el); #c1(TGIF2) c2(NP_001186444) c3 (homeobox protein TGIF2) c4(77654, 77655) c5(77656, 77657, 77658, 77659, 77660, 77661, 77662, 77663, 77664, 77665, 77666, 77667, 77668, 77669, 77670, 77671, 77672, 77673, 77674, 77675) c6( ) c7(jf, ZW, NT); #c1(TGIF2-C20orf24) c2(NP_001186464) c3(TGIF2-C20orf24 protein) c4(77676) c5(77677, 77678, 77679, 77680, 77681, 77682) c6( ) c7(jf, ZW, NT); #c1(TGIF2LX) c2(NP_620410) c3(homeobox protein TGIF2LX) c4(237575) c5(237576, 237577, 237578, 237579, 237580, 237581, 237582, 237583, 237584, 237585) c6( ) c7(vJ, ae, il, oe, ad); #c1(TGM1) c2(NP_000350) c3(protein-glutamine gamma-glutamyltransferase K) c4(7653, 7654, 7655, 7656, 7657, 7658) c5(7659, 7660, 7661, 7662, 7663, 7664, 7665, 7666, 7667, 7668, 7669, 7670, 7671, 7672, 7673, 7674, 7675, 7676, 7677, 7678, 7679, 7680, 7681, 7682, 7683, 7684, 7685, 7686, 7687, 7688, 7689, 7690, 7691, 7692, 7693, 7694, 7695, 7696, 7697, 7698, 7699) c6( ) c7(xS, cLR, ml, rl, n, btu, ad, av, btD, ft, bnC, Tt, buR, aEJ, fn, dL, lQ, cLT, jj, ae, af, cLQ, oV, ff, LO, hl, cr, rM, hQ, en, eE, bz, iq, bjw, rR, eM, Ts, lV, U, NI, at, cLS, act); #c1(TGM2) c2(NP_945189) c3 (protein-glutamine gamma-glutamyltransferase 2 isoform b) c4( ) c5(260717, 260718, 260719, 260720, 260721, 260722, 260723, 260724, 260725, 260726, 260727, 260728) c6( ) c7(bM, T, aC, al, ci, aR, ip, x, aE, K, eL, cp, eG, dH, aW, g, cm, aAW, biU, eM, mx, U, QF, m, k, act, Zo, pG, ht, n, aj, fr, ju, PD, ZN, he, Ha, A, lb, bJi, kp, dw, btK, vD, dB, Qu, bo, Po, B, bL, dA, cz, q, J, eA, nv, Qg, aT, cy, btE, bK, jj, ae, Lu, s, il, Y, r, lo, en, t, rR, ac, c, pZ, lV, cLU, mf, np, VB, id, Fb, fu, ad, HD, bj, WZ, d, lU, asf, ff, xs, hp, app, iV, bz, kN, Bv, Hs, cP, be, BA); #c1(TGM2) c2(XP_011527331) c3(protein-glutamine gamma-glutamyltransferase 2 isoform X1) c4(321954, 321955, 321956, 321957, 321958) c5(321959, 321960, 321961, 321962, 321963, 321964, 321965, 321966, 321967, 321968, 321969, 321970, 321971, 321972, 321973, 321974, 321975, 321976) c6( ) c7(bM, T, aC, al, ci, aR, ip, x, aE, K, eL, cp, eG, dH, aW, g, cm, aAW, biU, eM, mx, U, QF, m, k, act, Zo, pG, ht, n, aj, fr, ju, PD, ZN, he, Ha, A, lb, bJi, kp, dw, btK, vD, dB, Qu, bo, Po, B, bL, dA, cz, q, J, eA, nv, Qg, aT, cy, btE, bK, jj, ae, Lu, s, il, Y, r, lo, en, t, rR, ac, c, pZ, lV, cLU, mf, np, VB, id, Fb, fu, ad, hD, bj, WZ, d, lU, asf, ff, xs, hp, app, iV, bz, kN, Bv, Hs, cP, be, BA); #c1(TGM3) c2(NP_003236) c3(protein-glutamine gamma-glutamyltransferase E) c4(136652, 136653) c5(136654, 136655, 136656, 136657, 136658, 136659, 136660, 136661, 136662, 136663, 136664, 136665) c6( ) c7(T, sS, rR, lV, ff, ht, cr, aT, lQ, en, hu, eE); #c1(TGM4) c2(NP_003232) c3(protein-glutamine gamma-glutamyltransferase 4) c4(136638) c5(136639, 136640, 136641, 136642, 136643, 136644) c6( ) c7(vH, uz, ae, ad); #c1(TGM5) c2(NP_004236) c3(protein-glutamine gamma-glutamyltransferase 5 isoform 2) c4(144509) c5(144510, 144511, 144512, 144513, 144514, 144515) c6( ) c7(av, aFQ, KB, cr, rf, lO, lQ, eE, cLV); #c1(TGM5) c2(NP_963925) c3(protein-glutamine gamma-glutamyltransferase 5 isoform 1) c4(262577, 262578) c5(262579, 262580, 262581, 262582, 262583, 262584, 262585, 262586, 262587, 262588, 262589, 262590) c6( ) c7(av, aFQ, kB, cr, rf, lO, lQ, eE, cLV); #c1(TGM6) c2(NP_001241663) c3(protein-glutamine gamma-glutamyltransferase 6 isoform 2) c4(89021) c5(89022, 89023, 89024, 89025, 89026, 89027) c6( ) c7(ac, kB, bJi, rf, lO, bt, aR, CLW); #c1(TGM6) c2(NP_945345) c3(protein-glutamine gamma-glutamyltransferase 6 isoform 1) c4(260843) c5(260844, 260845, 260846, 260847, 260848, 260849) c6( ) c7(ac, kB, bJi, rf, lO, bt, aR, cLW); #c1(TGM7) c2(NP_443187) c3(protein-glutamine gamma-glutamyltransferase Z) c4(229760) c5(229761, 229762, 229763, 229764, 229765, 229766) c6( ) c7(aW, aT); #c1(TGOLN2) c2(NP_001193770) c3(trans-Golgi network integral membrane protein 2 isoform 3 precursor) c4(82565) c5(82566, 82567, 82568, 82569, 82570, 82571) c6( ) c7(hz, bS, cA, afu); #c1(TGOLN2) c2(NP_001193773) c3(trans-Golgi network integral membrane protein 2 isoform 4 precursor) c4(82572, 82573) c5(82574, 82575, 82576, 82577, 82578, 82579, 82580, 82581, 82582, 82583, 82584, 82585) c6( ) c7(hz, bS, cA, afu); #c1(TGOLN2) c2(NP_006455) c3(trans-Golgi network integral membrane protein 2 isoform 1 precursor) c4(165354) c5(165355, 165356, 165357, 165358, 165359, 165360) c6( ) c7(hz, bS, cA, afu); #c1(TGS1) c2(NP_079107) c3(trimethylguanosine synthase) c4(217054) c5(217055, 217056, 217057, 217058, 217059, 217060) c6( ) c7(cm, T, bz, dA); #c1(TH) c2(NP_000351) c3(tyrosine 3-monooxygenase isoform b) c4(7700) c5(7701, 7702, 7703, 7704, 7705, 7706) c6( ) c7(bM, T, os, Tb, e, aAx, Jd, qj, aR, aji, fm, aU, K, cm, uC, mZ, yl, Kv, QF, uJ, act, aWX, rt, bfb, lT, RX, aw, adk, aj, ct, nA, cd, eW, Ak, h, aNU, OM, mY, mV, he, bAl, dx, ml, bmc, A, cly, fl, mX, cv, bJ, cA, bo, sZ, B, mT, bL, oe, aOH, aA, cz, mS, wo, v, ON, avO, wy, V, J, hb, HC, bK, bq, an, ae, af, eu, ak, kl, Qr, yL, akt, lo, bN, Qi, pa, bAn, ac, HK, VB, wi, ha, uG, jh, acU, oZ, bS, uu, qi, aue, xt, ad, Tg, agd, mW, sO, bma, Sb, na, Gv, aF, EZ, qa, gZ, ei, pC); #c1(TH) c2(NP_954986) c3(tyrosine 3-monooxygenase isoform a) c4(261506) c5(261507, 261508, 261509, 261510, 261511, 261512, 261513) c6( ) c7(bM, T, os, Tb, e, aAx, Jd, qj, aR, aji, fm, aU, K, cm, uC, mZ, yl, Kv, QF, uJ, act, aWX, rt, bfb, lT, RX, aw, adk, aj, ct, nA, cd, eW, Ak, h, aNU, OM, mY, mV, he, bAl, dx, ml, bmc, A, cly, fQ, mX, cv, bJ, cA, bo, sZ, B, mT, bL, oe, aOH, aA, cz, mS, wo, v, ON, avO, wy, V, J, hb, HC, bK, bq, an, ae, af, eu, ak, kl, Qr, yL, akt, lo, bN, Qi, pa, bAn, ac, HK, VB, wi, ha, uG, jh, acU, oZ, bS, uu, qi, aue, xt, ad, Tg, agd, mW, sO, bma, Sb, na, Gv, aF, EZ, qa, gZ, ei, pC); #c1(TH) c2(NP_954987) c3(tyrosine 3-monooxygenase isoform c) c4(261514) c5(261515, 261516, 261517, 261518, 261519, 261520) c6( ) c7(bM, T, os, Tb, e, aAx, Jd, qj, aR, aji, fm, aU, K, cm, uC, mZ, yl, Kv, QF, uJ, act, aWX, rt, bfb, lT, RX, aw, adk, aj, ct, nA, cd, eW, Ak, h, aNU, OM, mY, mV, he, bAl, dx, mU, bmc, A, cly, fQ, mX, cv, bJ, cA, bo, sZ, B, mT, bl, oe, aOH, aA, cz, mS, wo, v, ON, avO, wy, V, J, hb, HC, bK, bq, an, ae, af, eu, ak, kl, Qr, yL, akt, lo, bN, Qi, pa, bAn, ac, HK, VB, wi, ha, uG, jh, acU, oZ, bS, uu, qi, aue, xt, ad, Tg, agd, mW, sO, bma, Sb, na, Gv, aF, EZ, qa, gZ, ei, pC); #c1(TH) c2(XP_011518637) c3(tyrosine 3-monooxygenase isoform X1) c4(312957) c5(312958, 312959, 312960, 312961, 312962, 312963) c6( ) c7(bM, T, os, Tb, e, aAx, Jd, qj, aR, aji, fm, aU, K, cm, uC, mZ, yl, Kv, QF, uJ, act, aWX, rt, bfb, lT, RX, aw, adk, aj, ct, nA, cd, eW, Ak, h, aNU, OM, mY, mV, he, bAl, dx, mU, bmc, A, cly, fl, mX, cv, bJ, cA, bo, sZ, B, mT, bL, oe, aOH, aA, cz, mS, wo, v, ON, avO, wy, V, J, hb, HC, bK, bq, an, ae, af, eu, ak, kl, Qr, yL, akt, lo, bN, Qi, pa, bAn, ac, HK, VB, wi, ha, uG, jh, acU, oZ, bS, uu, qi, aue, xt, ad, Tg, agd, mW, sO, bma, Sb, na, Gv, aF, EZ, qa, gZ, ei, pC); #c1(THADA) c2(NP_001077422) c3(thyroid adenoma-associated protein isoform a) c4(37835, 37836) c5(37837, 37838, 37839, 37840, 37841, 37842, 37843, 37844, 37845, 37846, 37847, 37848, 37849, 37850, 37851, 37852, 37853, 37854, 37855, 37856, 37857, 37858) c6( ) c7(bL, kl, s, aZ, h, aj, v, AN, ci, Nc, cl, J, bvU, ad); #c1(THADA) c2(NP_001258573) c3(thyroid adenoma-associated protein isoform c) c4(96160) c5(96161, 96162, 96163, 96164, 96165, 96166) c6( ) c7(bL, kl, s, aZ, h, aj, v, AN, ci, Nc, cl, J, bvU, ad); #c1(THADA) c2(XP_006712128) c3(thyroid adenoma-associated protein isoform X5) c4(286417) c5(286418, 286419, 286420, 286421, 286422, 286423) c6( ) c7(bL, kl, s, aZ, h, aj, v, AN, ci, Nc, cl, J, bvU, ad); #c1(THADA) c2(XP_006712131) c3(thyroid adenoma-associated protein isoform X8) c4(286424) c5( ) c6( ) c7(bL, kl, s, aZ, h, aj, v, AN, ci, Nc, cl, J, bvU, ad); #c1(THAP1) c2(NP_060575) c3(THAP domain-containing protein 1 isoform 1) c4(196630) c5(196631, 196632, 196633, 196634, 196635, 196636, 196637) c6( ) c7(aOQ, an, Wk, akt, aGM, aU, Wl, bAm, bAq, aUR, aUZ, Wm, cLX, bDO, aH); #c1(THAP1) c2(NP_945354) c3(THAP domain-containing protein 1 isoform 2) c4(260893) c5(260894, 260895, 260896, 260897, 260898) c6( ) c7(aOQ, an, Wk, akt, aGM, aU, Wl, bAm, bAq, aUR, aUZ, Wm, cLX, bDO, aH); #c1(THAP10) c2(NP_064532) c3(THAP domain-containing protein 10) c4(202878) c5(202879, 202880, 202881, 202882, 202883, 202884) c6( ) c7(aW, aT); #c1(THAP11) c2(NP_065190) c3(THAP domain-containing protein 11) c4(204508) c5(204509, 204510, 204511, 204512, 204513, 204514) c6( ) c7(bB, aq, bE, B); #c1(THAP2) c2(NP_113623) c3(THAP domain-containing protein 2) c4(221785) c5(221786, 221787, 221788, 221789, 221790, 221791) c6( ) c7(j); #c1(THAP6) c2(NP_653322) c3(THAP domain-containing protein 6) c4(239584) c5(239585, 239586, 239587, 239588, 239589, 239590, 239591, 239592, 239593, 239594) c6( ) c7(cd); #c1(THAP6) c2(XP_005262831) c3(THAP domain-containing protein 6 isoform X5) c4( ) c5(278113, 278114, 278115, 278116, 278117, 278118, 278119, 278120, 278121, 278122, 278123, 278124) c6( ) c7(cd); #c1(THAP6) c2(XP_006714172) c3(THAP domain-containing protein 6 isoform X4) c4(287636) c5( ) c6( ) c7(cd); #c1(THBD) c2(NP_000352) c3(thrombomodulin precursor) c4(7707, 7708) c5(7709, 7710, 7711, 7712, 7713, 7714, 7715, 7716, 7717, 7718, 7719, 7720, 7721, 7722, 7723, 7724, 7725, 7726, 7727, 7728, 7729, 7730, 7731, 7732, 7733, 7734, 7735, 7736, 7737, 7738, 7739, 7740, 7741, 7742, 7743, 7744, 7745, 7746, 7747, 7748, 7749, 7750, 7751, 7752, 7753, 7754, 7755, 7756, 7757, 7758, 7759, 7760, 7761, 7762, 7763, 7764, 7765, 7766, 7767, 7768, 7769, 7770, 7771, 7772, 7773, 7774, 7775, 7776, 7777, 7778, 7779, 7780, 7781, 7782, 7783, 7784, 7785, 7786, 7787, 7788, 7789, 7790, 7791, 7792, 7793, 7794, 7795, 7796, 7797, 7798, 7799, 7800, 7801, 7802, 7803, 7804, 7805, 7806, 7807, 7808) c6(7809) c7(bM, T, ea, if, e, al, eb, ame, ci, vh, M, K, hH, eL, hm, ih, uC, aUF, eM, wE, dW, wx, m, eg, P, dD, cLY, aw, bd, qN, qW, ai, eW, ql, bud, cU, dL, wC, aJ, fQ, ahJ, kT, bm, E, B, bL, eR, DD, aGu, DV, aA, sM, cz, fF, j, C, mj, uT, cy, jj, bq, ae, af, bA, iZ, Y, DN, aDm, en, t, wg, ac, ig, AH, cS, dy, bt, hS, sY, qb, oR, eq, ad, bqs, Jm, cLZ, nH, Cj, jo, ff, eZ, dN, gE, aF, bz, ek, TA, DT, ag, gZ, aS, y, fh); #c1(THBS1) c2(NP_003237) c3(thrombospondin-1 precursor) c4(136666) c5(136667, 136668, 136669, 136670, 136671, 136672, 136673) c6( ) c7(ID, T, aC, Tb, ea, if, e, al, aHx, WV, fd, bBR, bB, lR, ci, aR, ip, afr, aE, Bc, aEJ, rB, K, eL, cp, mH, ly, DA, aW, lh, g, eF, cm, uC, iP, qJ, eM, nB, iA, vy, cj, aZo, Hb, m, cE, dD, aw, fN, aj, fr, qN, zP, cd, ai, Bl, gd, KH, Ne, lG, aOM, dL, h, wC, asp, aiB, aq, kp, dw, op, lx, KT, Vy, cMb, hL, te, aiS, ph, Pr, B, it, cn, cir, DD, atB, Ju, WT, aA, v, j, Qo, V, q, J, eA, eJ, DJ, lg, aT, cy, jj, bq, kU, ae, jf, s, nO, oV, Y, r, aJl, lo, en, cl, aaO, t, pb, ck, nM, ac, iM, il, WU, agf, wR, mf, yH, fD, sY, id, aZ, rl, jh, dG, eq, ad, aiN, XB, Jm, dP, av, hd, Dq, aCJ, oj, uD, WW, uX, d, je, Di, qt, ff, eH, Ce, rM, dN, kD, aF, bz, aJx, DT, RV, zr, bE, cMa, gZ, aS, lC, KS, iu, y, BA); #c1(THBS1) c2(XP_011520273) c3(thrombospondin-1 isoform X2) c4(315113) c5(315114, 315115, 315116, 315117, 315118, 315119, 315120, 315121, 315122) c6( ) c7(ID, T, aC, Tb, ea, if, e, al, aHx, WV, fd, bBR, bB, lR, ci, aR, ip, afr, aE, Bc, aEJ, rB, K, eL, cp, mH, ly, DA, aW, lh, g, eF, cm, uC, iP, qJ, eM, nB, iA, vy, cj, aZo, Hb, m, cE, dD, aw, fN, aj, fr, qN, zP, cd, ai, Bl, gd, KH, Ne, lG, aOM, dL, h, wC, asp, aiB, aq, kp, dw, op, lx, KT, Vy, cMb, hl, te, aiS, ph, Pr, B, it, cn, cir, DD, atB, Ju, WT, aA, v, j, Qo, V, q, J, eA, eJ, DJ, lg, aT, cy, jj, bq, kU, ae, jf, s, nO, oV, Y, r, aJl, lo, en, cl, aaO, t, pb, ck, nM, ac, iM, il, WU, agf, wR, mf, yH, fD, sY, id, aZ, rl, jh, dG, eq, ad, aiN, XB, Jm, dP, av, hd, Dq, aCJ, oj, uD, WW, uX, d, je, Di, qt, ff, eH, Ce, rM, dN, KD, aF, bz, aJx, DT, RV, zr, bE, cMa, gZ, aS, lC, KS, iu, y, BA); #c1(THBS2) c2(NP_003238) c3(thrombospondin-2 precursor) c4(136674, 136675, 136676) c5(136677, 136678, 136679, 136680, 136681, 136682, 136683, 136684, 136685, 136686, 136687, 136688, 136689, 136690, 136691, 136692, 136693, 136694) c6( ) c7(bL, CX, T, dD, dA, eR, iB, yk, if, aw, cz, bB, n, wV, fd, j, ci, eq, qN, cd, ad, eA, r, gd, aCV, aT, K, ae, aRs, aiB, s, cW, aW, Y, oV, ff, en, cl, g, t, op, Ek, aik, aDX, eM, bE, AM, hL, m, be, ei, fy); #c1(THBS3) c2(NP_001239537) c3(thrombospondin-3 isoform 3 precursor) c4(88477, 88478) c5(88479, 88480, 88481, 88482, 88483, 88484, 88485, 88486, 88487, 88488, 88489, 88490, 88491, 88492, 88493, 88494, 88495, 88496, 88497, 88498, 88499, 88500, 88501) c6(88502, 88503, 88504, 88505, 88506, 88507) c7(eF, eH); #c1(THBS3) c2(NP_009043) c3(thrombospondin-3 isoform 1 precursor) c4(170370, 170371, 170372) c5(170373, 170374, 170375, 170376, 170377, 170378, 170379, 170380) c6(170381, 170382) c7(eF, eH); #c1(THBS3) c2(XP_006711561) c3(thrombospondin-3 isoform X2) c4( ) c5(286125, 286126, 286127, 286128, 286129, 286130, 286131, 286132, 286133) c6(286134, 286135) c7(eF, eH); #c1(THBS4) c2(NP_003239) c3(thrombospondin-4 precursor) c4(136695) c5(136696, 136697, 136698, 136699, 136700, 136701, 136702, 136703) c6(136704, 136705) c7(bM, sY, T, dD, eR, ea, e, aw, cz, j, ci, eq, q, K, H, af, bkS, s, r, g, t, uC, ac, dh, aTW, L); #c1(THEG) c2(NP_057669) c3(testicular haploid expressed gene protein isoform 1) c4(192264) c5(192265, 192266, 192267, 192268, 192269) c6( ) c7(x, T, gU, cqN, ac, dB, Y, hF, Pl, aA, vH, cm, aF, m, k, bu, t, pt, rc, UE); #c1(THEG) c2(NP_954672) c3(testicular haploid expressed gene protein isoform 2) c4(261233) c5(261234, 261235, 261236, 261237, 261238, 261239) c6( ) c7(x, T, gU, cqN, ac, dB, Y, hF, Pl, aA, vH, cm, aF, m, k, bu, t, pt, rc, UE); #c1(THEG) c2(XP_011526351) c3(testicular haploid expressed gene protein isoform X1) c4( ) c5(320649) c6( ) c7(x, T, gU, cqN, ac, dB, Y, hF, Pl, aA, vH, cm, aF, m, k, bu, t, pt, rc, UE); #c1(THEM4) c2(NP_444283) c3(acyl-coenzyme A thioesterase THEM4) c4(230296) c5(230297, 230298, 230299, 230300, 230301, 230302) c6( ) c7(bL, d, B, eA, bz, eJ); #c1(THEM5) c2(NP_872384) c3(acyl-coenzyme A thioesterase THEM5) c4(256289, 256290) c5(256291, 256292, 256293, 256294, 256295, 256296, 256297, 256298, 256299, 256300, 256301, 256302) c6( ) c7(fc); #c1(THEM5) c2(XP_011507723) c3(acyl-coenzyme A thioesterase THEM5 isoform X1) c4( ) c5(297808) c6( ) c7(fc); #c1(THEM6) c2(NP_057731) c3(protein THEM6 precursor) c4(192549) c5(192550, 192551, 192552, 192553, 192554, 192555, 192556, 192557) c6(192558, 192559) c7(q, r); #c1(THEMIS) c2(NP_001010923) c3(protein THEMIS isoform 2) c4(24171, 24172, 24173) c5(24174, 24175, 24176, 24177, 24178, 24179, 24180, 24181, 24182, 24183, 24184, 24185, 24186, 24187, 24188, 24189, 24190, 24191) c6( ) c7(T, A, cc, ig, aM, LZ, aT, v, hp); #c1(THEMIS) c2(NP_001158157) c3(protein THEMIS isoform 1) c4(63209) c5(63210, 63211, 63212, 63213, 63214, 63215, 63216, 63217) c6( ) c7(T, A, cc, ig, aM, LZ, aT, v, hp); #c1(THEMIS) c2(XP_011534115) c3(protein THEMIS isoform X1) c4(330355) c5( ) c6( ) c7(T, A, cc, ig, aM, LZ, aT, v, hp); #c1(THEMIS2) c2(XP_011540747) c3(protein THEMIS2 isoform X1) c4(339320) c5(339321, 339322, 339323, 339324, 339325, 339326) c6( ) c7(ck, T, AM, aW, hL, al, m, cl, t); #c1(THG1L) c2(NP_060342) c3(probable tRNA (His) guanylyltransferase) c4(195499, 195500) c5(195501, 195502, 195503, 195504, 195505, 195506) c6( ) c7(K, Hb); #c1(THNSL1) c2(NP_079114) c3(threonine synthase-like 1) c4(217092) c5(217093, 217094, 217095, 217096, 217097, 217098, 217099, 217100, 217101, 217102) c6( ) c7(cz); #c1(THNSL2) c2(NP_001231605) c3(threonine synthase-like 2 isoform 2) c4(87325) c5(87326, 87327, 87328, 87329, 87330, 87331, 87332, 87333, 87334, 87335, 87336, 87337, 87338, 87339) c6(87340, 87341, 87342, 87343) c7(uz, y, zf); #c1(THNSL2) c2(NP_001231607) c3(threonine synthase-like 2 isoform 3) c4( ) c5(87344, 87345, 87346, 87347, 87348, 87349, 87350) c6(87351, 87352) c7(uz, y, zf); #c1(THNSL2) c2(NP_060741) c3(threonine synthase-like 2 isoform 1) c4(197569) c5(197570, 197571, 197572, 197573, 197574, 197575, 197576, 197577, 197578, 197579, 197580, 197581, 197582, 197583) c6(197584, 197585, 197586, 197587) c7(uz, y, zf); #c1(THNSL2) c2(XP_005264460) c3(threonine synthase-like 2 isoform X9) c4(278941, 278942) c5(278943, 278944, 278945, 278946, 278947, 278948, 278949, 278950, 278951) c6(278952, 278953) c7(uz, y, zf); #c1(THNSL2) c2(XP_005264461) c3(threonine synthase-like 2 isoform X10) c4(278954) c5( ) c6( ) c7(uz, y, zf); #c1(THNSL2) c2(XP_006712107) c3(threonine synthase-like 2 isoform X13) c4( ) c5(286402, 286403, 286404, 286405, 286406, 286407, 286408, 286409) c6(286410, 286411) c7(uz, y, zf); #c1(THOC1) c2(NP_005122) c3(THO complex subunit 1) c4(153064) c5(153065, 153066, 153067, 153068, 153069, 153070) c6( ) c7(ae, T, aC, aE, fR, aW, aT, n, PQ, m, bK, g, t, bz, eJ, ad); #c1(THOC2) c2(NP_001075019) c3(THO complex subunit 2) c4(37162) c5(37163, 37164, 37165, 37166, 37167, 37168, 37169, 37170, 37171, 37172, 37173, 37174, 37175) c6( ) c7(Dr, bS); #c1(THOC2) c2(XP_011529673) c3(THO complex subunit 2 isoform X4) c4(325314) c5( ) c6( ) c7(Dr, bS); #c1(THOC2) c2(XP_011529676) c3(THO complex subunit 2 isoform X6) c4(325315) c5(325316, 325317, 325318, 325319, 325320, 325321, 325322, 325323, 325324, 325325, 325326) c6( ) c7(Dr, bS); #c1(THOC5) c2(NP_001002878) c3(THO complex subunit 5 homolog) c4(19687) c5(19688, 19689, 19690, 19691, 19692, 19693, 19694, 19695, 19696, 19697, 19698, 19699, 19700, 19701, 19702, 19703, 19704, 19705, 19706, 19707, 19708, 19709, 19710, 19711, 19712, 19713, 19714, 19715, 19716, 19717, 19718, 19719, 19720, 19721, 19722, 19723) c6( ) c7(ck, il); #c1(THOC6) c2(XP_005261856) c3(THO complex subunit 5 homolog isoform X1) c4(277643, 277644, 277645) c5( ) c6( ) c7(ck, il); #c1(THOC6) c2(NP_001135822) c3(THO complex subunit 6 homolog isoform 2) c4(53349) c5(53350, 53351, 53352, 53353, 53354, 53355) c6( ) c7(cMc); #c1(THOC6) c2(NP_077315) c3(THO complex subunit 6 homolog isoform 1) c4(215209) c5(215210, 215211, 215212, 215213, 215214, 215215) c6( ) c7(cMc); #c1(THOP1) c2(NP_003240) c3(thimet oligopeptidase) c4(136706, 136707) c5(136708, 136709, 136710, 136711, 136712, 136713) c6( ) c7(eY, ae, T, xc, aaH, Or, er, bm, cj, bK, ci, il, V, bz, fK, ad); #c1(THOP1) c2(XP_011526530) c3(thimet oligopeptidase isoform X1) c4( ) c5(320834, 320835, 320836, 320837, 320838, 320839) c6( ) c7(eY, ae, T, xc, aaH, Or, er, bm, cj, bK, ci, il, V, bz, fK, ad); #c1(THPO) c2(NP_001276932) c3(thrombopoietin isoform 5) c4( ) c5(108680, 108681, 108682) c6( ) c7(T, os, e, al, ci, x, Vp, xy, K, biP, DY, hl, bw, uC, cMd, iT, nB, cj, dW, k, hh, aeA, aw, br, cc, RN, h, bOY, hn, dL, DU, Hz, fQ, aq, aGd, pm, iO, cA, kz, pg, B, DM, dA, eR, jx, hz, j, C, Wa, biO, bKE, mP, dz, bK, iU, cMe, t, Cv, ck, pa, AH, hr, JH, gT, bt, vi, gV, sY, jh, bs, oZ, bLo, hF, av, hd, F, CJ, bpp, cMf, oW, hD, iY, yF, hG, et, KD, ok, gk, arB, pc, brt); #c1(THPO) c2(NP_001276951) c3(thrombopoietin isoform 2 precursor) c4(108703) c5(108704, 108705, 108706, 108707, 108708, 108709, 108710, 108711) c6(108712, 108713) c7(T, os, e, al, ci, x, Vp, xy, K, biP, DY, hl, bw, uC, cMd, iT, nB, cj, dW, k, hh, aeA, aw, br, cc, RN, h, bOY, hn, dL, DU, Hz, fQ, aq, aGd, pm, iO, cA, kz, pg, B, DM, dA, eR, jx, hz, j, C, Wa, biO, bKE, mP, dz, bK, il, cMe, t, Cv, ck, pa, AH, hr, JH, gT, bt, vi, gV, sY, jh, bs, oZ, bLo, hF, av, hd, F, CJ, bpp, cMf, oW, hD, iY, yF, hG, et, kD, ok, gk, arB, pc, brt); #c1(THPO) c2(NP_001276956) c3(thrombopoietin isoform 4 precursor) c4(108716) c5(108717, 108718, 108719, 108720, 108721, 108722, 108723, 108724, 108725) c6(108726, 108727) c7(T, os, e, al, ci, x, Vp, xy, K, biP, DY, hl, bw, uC, cMd, iT, nB, cj, dW, k, hh, aeA, aw, br, cc, RN, h, bOY, hn, dL, DU, Hz, fQ, aq, aGd, pm, iO, cA, kz, pg, B, DM, dA, eR, jx, hz, j, C, Wa, biO, bKE, mP, dz, bK, il, cMe, t, Cv, ck, pa, AH, hr, JH, gT, bt, vi, gV, sY, jh, bs, oZ, bLo, hF, av, hd, F, CJ, bpp, cMf, oW, hD, iY, yF, hG, et, KD, ok, gk, arB, pc, brt); #c1(THPO) c2(NP_001276957) c3(thrombopoietin isoform 1 precursor) c4(108728, 108729) c5(108730, 108731, 108732, 108733, 108734, 108735, 108736, 108737, 108738, 108739, 108740, 108741, 108742, 108743, 108744, 108745, 108746, 108747, 108748, 108749, 108750, 108751, 108752, 108753, 108754, 108755, 108756, 108757, 108758, 108759, 108760, 108761, 108762, 108763, 108764, 108765, 108766, 108767, 108768, 108769, 108770, 108771, 108772, 108773, 108774, 108775, 108776, 108777, 108778, 108779, 108780, 108781, 108782, 108783, 108784, 108785, 108786, 108787, 108788, 108789, 108790, 108791, 108792, 108793, 108794, 108795, 108796, 108797, 108798, 108799, 108800, 108801, 108802, 108803, 108804, 108805, 108806, 108807, 108808, 108809, 108810, 108811, 108812, 108813, 108814, 108815, 108816, 108817, 108818, 108819, 108820, 108821, 108822, 108823, 108824, 108825, 108826, 108827, 108828, 108829, 108830, 108831, 108832, 108833, 108834, 108835, 108836) c6(108837, 108838, 108839, 108840, 108841, 108842, 108843) c7(T, os, e, al, ci, x, Vp, xy, K, biP, DY, hl, bw, uC, cMd, iT, nB, cj, dW, k, hh, aeA, aw, br, cc, RN, h, bOY, hn, dL, DU, Hz, fQ, aq, aGd, pm, iO, cA, kz, pg, B, DM, dA, eR, jx, hz, j, C, Wa, biO, bKE, mP, dz, bK, iU, cMe, t, Cv, ck, pa, AH, hr, JH, gT, bt, vi, gV, sY, jh, bs, oZ, bLo, hF, av, hd, F, CJ, bpp, cMf, oW, hD, iY, yF, hG, et, kD, ok, gk, arB, pc, brt); #c1(THRA) c2(NP_001177847) c3(thyroid hormone receptor alpha isoform 3) c4(73096) c5(73097, 73098, 73099, 73100, 73101, 73102) c6( ) c7(T, aA, cz, v, j, hX, dP, hd, no, bfJ, aT, jj, af, qw, s, qt, aW, gT, cMg, aq, aF, t, dr, Ew, GU, nB, eM, wk, m, hE, B); #c1(THRA) c2(NP_003241) c3(thyroid hormone receptor alpha isoform 2) c4(136714) c5(136715, 136716, 136717, 136718, 136719, 136720, 136721, 136722, 136723, 136724, 136725) c6( ) c7(T, aA, cz, v, j, hX, dP, hd, no, bfJ, aT, jj, af, qw, s, qt, aW, gT, cMg, aq, aF, t, dr, Ew, GU, nB, eM, wk, m, hE, B); #c1(THRA) c2(NP_955366) c3(thyroid hormone receptor alpha isoform 1) c4(261570) c5(261571, 261572, 261573, 261574, 261575, 261576, 261577, 261578, 261579, 261580, 261581) c6( ) c7(T, aA, cz, v, j, hX, dP, hd, no, bfJ, aT, jj, af, qw, s, qt, aW, gT, cMg, aq, aF, t, dr, Ew, GU, nB, eM, wk, m, hE, B); #c1(THRB) c2(NP_001239563) c3(thyroid hormone receptor beta) c4(88527, 88528) c5(88529, 88530, 88531, 88532, 88533, 88534, 88535, 88536, 88537, 88538, 88539, 88540, 88541, 88542, 88543, 88544, 88545, 88546, 88547, 88548, 88549, 88550, 88551) c6( ) c7(T, gU, dA, ht, bzb, cMj, hz, aj, v, fr, ci, ml, qN, J, av, cF, ft, hd, xy, py, Db, aT, hD, hu, cMi, af, yF, aW, AQ, Az, cMh, atf, g, dr, ck, pv, es, qJ, nM, sn, iA, nB, cMk, wk, hr, hR, B, VB); #c1(THRSP) c2(NP_003242) c3(thyroid hormone-inducible hepatic protein) c4(136726) c5(136727, 136728, 136729, 136730, 136731, 136732) c6( ) c7(zQ, aW, aT, n, v, ci, zP, B); #c1(THSD1) c2(NP_061146) c3(thrombospondin type-1 domain-containing protein 1 isoform 1 precursor) c4(199512, 199513, 199514) c5(199515, 199516, 199517, 199518, 199519, 199520, 199521, 199522, 199523, 199524, 199525, 199526, 199527, 199528, 199529, 199530, 199531, 199532, 199533, 199534, 199535, 199536, 199537, 199538) c6(199539, 199540, 199541, 199542, 199543, 199544) c7(T, it, q, hu, r); #c1(THSD1) c2(NP_954872) c3(thrombospondin type-1 domain-containing protein 1 isoform 2 precursor) c4(261485) c5(261486, 261487, 261488, 261489, 261490, 261491, 261492, 261493) c6(261494, 261495) c7(T, it, q, hu, r); #c1(THSD4) c2(NP_001273358) c3(thrombospondin type-1 domain-containing protein 4 isoform 2 precursor) c4(104733, 104734) c5(104735, 104736, 104737, 104738, 104739, 104740, 104741, 104742, 104743, 104744, 104745, 104746, 104747, 104748, 104749, 104750) c6(104751, 104752, 104753, 104754) c7(yE, at, Dp, gv, lB, bA); #c1(THSD4) c2(NP_079093) c3(thrombospondin type-1 domain-containing protein 4 isoform 1 precursor) c4(216990) c5(216991, 216992, 216993, 216994, 216995, 216996, 216997, 216998, 216999, 217000, 217001, 217002, 217003, 217004) c6(217005, 217006, 217007, 217008) c7(yE, at, Dp, gv, lB, bA); #c1(THSD7A) c2(NP_056019) c3(thrombospondin type-1 domain-containing protein 7A precursor) c4(185165, 185166) c5(185167, 185168, 185169, 185170, 185171, 185172, 185173, 185174, 185175, 185176, 185177, 185178) c6( ) c7(abV, yE, EQ, bK, fq, bA); #c1(THYN1) c2(NP_001032381) c3(thymocyte nuclear protein 1 isoform 2) c4( ) c5(31280, 31281, 31282, 31283, 31284) c6( ) c7(ad); #c1(THYN1) c2(NP_001032382) c3(thymocyte nuclear protein 1 isoform 1) c4( ) c5(31285, 31286, 31287, 31288, 31289, 31290, 31291, 31292, 31293, 31294, 31295) c6( ) c7(ad); #c1(THYN1) c2(XP_005271584) c3(thymocyte nuclear protein 1 isoform X1) c4(283481) c5(283482, 283483, 283484) c6( ) c7(ad); #c1(THYN1) c2(XP_005271585) c3(thymocyte nuclear protein 1 isoform X2) c4(283485) c5(283486) c6( ) c7(ad); #c1(TIA1) c2(NP_071320) c3(nucleolysin TIA-1 isoform p40 isoform 1) c4(210942) c5(210943, 210944, 210945, 210946, 210947, 210948) c6( ) c7(aol, T, fM, fR, mr, Ol, iW, jh, hX, kf, K, li, lb, lo, vs, bji, Bu, c, cj, zv, y, B); #c1(TIA1) c2(NP_071505) c3(nucleolysin TIA-1 isoform p40 isoform 2) c4(211791) c5(211792, 211793, 211794, 211795, 211796, 211797) c6( ) c7(aoU, T, fM, fR, mr, Ol, iW, jh, hX, kf, K, li, lb, lo, vs, bji, Bu, c, cj, zv, y, B); #c1(TIA1) c2(XP_011531379) c3(nucleolysin TIA-1 isoform p40 isoform X1) c4(327383) c5(327384, 327385, 327386, 327387, 327388, 327389) c6( ) c7(aol, T, fM, fR, mr, Ol, iW, jh, hX, kf, K, li, lb, lo, vs, bji, Bu, c, cj, zv, y, B); #c1(TIAF1) c2(NP_004731) c3(TGFB1-induced anti-apoptotic factor 1) c4(149148) c5(149149, 149150, 149151, 149152, 149153, 149154, 149155, 149156, 149157) c6( ) c7(T, af, K); #c1(TIAL1) c2(NP_001029097) c3(nucleolysin TIAR isoform 2) c4(30757) c5(30758, 30759, 30760, 30761, 30762, 30763) c6( ) c7(fR, aT, fe, lo, vs, K); #c1(TIAL1) c2(NP_003243) c3(nucleolysin TIAR isoform 1) c4(136733, 136734, 136735) c5(136736, 136737, 136738, 136739, 136740, 136741, 136742, 136743, 136744, 136745, 136746, 136747, 136748, 136749, 136750, 136751, 136752, 136753) c6( ) c7(fR, aT, fe, lo, vs, K); #c1(TIAL1) c2(XP_005270167) c3(nucleolysin TIAR isoform X2) c4(283195) c5(283196, 283197, 283198, 283199, 283200, 283201) c6( ) c7(fR, aT, fe, lo, vs, K); #c1(TIAM1) c2(XP_005261095) c3(T-lymphoma invasion and metastasis-inducing protein 1 isoform X1) c4(277183, 277184, 277185, 277186) c5(277187, 277188, 277189, 277190, 277191, 277192, 277193, 277194, 277195, 277196, 277197, 277198) c6( ) c7(T, aC, jh, aA, v, fr, ci, PJ, q, ad, hX, aE, aT, h, cp, De, B, aW, Y, r, aq, aF, aZg, f, cm, bP, c, bE, hk, bt, it, X); #c1(TIAM1) c2(XP_005261097) c3(T-lymphoma invasion and metastasis-inducing protein 1 isoform X2) c4( ) c5(277199, 277200, 277201, 277202, 277203, 277204, 277205, 277206, 277207, 277208, 277209, 277210) c6( ) c7(T, aC, jh, aA, v, fr, ci, PJ, q, ad, hX, aE, aT, h, cp, De, B, aW, Y, r, aq, af, aZg, f, cm, bP, c, bE, hk, bt, it, X); #c1(TIAM2) c2(NP_036586) c3(T-lymphoma invasion and metastasis-inducing protein 2 isoform a) c4(174557, 174558, 174559, 174560, 174561, 174562, 174563, 174564, 174565, 174566, 174567) c5(174568, 174569, 174570, 174571, 174572, 174573, 174574, 174575, 174576, 174577, 174578, 174579, 174580, 174581, 174582, 174583, 174584, 174585, 174586, 174587, 174588, 174589, 174590, 174591, 174592, 174593, 174594, 174595, 174596, 174597, 174598, 174599, 174600, 174601, 174602, 174603, 174604, 174605, 174606, 174607, 174608, 174609, 174610, 174611, 174612, 174613, 174614, 174615, 174616, 174617, 174618, 174619, 174620, 174621, 174622, 174623, 174624, 174625, 174626, 174627, 174628, 174629, 174630, 174631, 174632, 174633, 174634, 174635, 174636, 174637) c6( ) c7(T, aq, bK, iR, lB, B); #c1(TICAM1) c2(NP_891549) c3(TIR domain-containing adapter molecule 1) c4(257266) c5(257267, 257268, 257269, 257270, 257271, 257272, 257273) c6( ) c7(Dw, cMl, jx, wV, C, aBl, atK, eW, aE, ds, cu, cy, eu, lo, hp, cMm, uF, pm, ac, NL, EY, fD, hh); #c1(TICAM2) c2(NP_067681) c3(TIR domain-containing adapter molecule 2) c4(209297) c5(209298, 209299) 209300, 209301, 209302, 209303) c6( ) c7(T, ea, if, fd, bB, ci, aE, il, Ok, K, eL, bc, xQ, yo, aW, as, cm, bw, GR, eM, nB, dc, ki, go, vA, cj, m, Hb, dD, NT, n, fr, qN, jz, h, dl, ge, aq, zZ, iO, bP, hL, hR, it, B, fM, aoE, aA, q, eA, XX, aT, dz, ae, af, jf, s, il, Y, r, en, cl, t, ik, aEp, ck, nM, iM, dy, EY, bt, On, hS, hl, Ke, pY, jh, eq, ad, hX, hd, ib, fb, d, je, et, ff, apb, aF, uF, bz, bE, aS, EB); #c1(TICRR) c2(NP_689472) c3(treslin) c4(242811) c5(242812, 242813, 242814, 242815, 242816, 242817) c6( ) c7(bX); #c1(TIFA) c2(NP_443096) c3(TRAF-interacting protein with FHA domain-containing protein A) c4(229384, 229385) c5(229386, 229387, 229388, 229389, 229390, 229391, 229392, 229393, 229394, 229395, 229396, 229397, 229398, 229399, 229400, 229401) c6( ) c7(fq); #c1(TIGD2) c2(NP_663761) c3(tigger transposable element-derived protein 2) c4(241537) c5(241538, 241539, 241540, 241541, 241542, 241543) c6( ) c7(j); #c1(TIGIT) c2(NP_776160) c3(T-cell immunoreceptor with lg and ITIM domains precursor) c4(250884, 250885) c5(250886, 250887, 250888, 250889, 250890, 250891, 250892, 250893, 250894, 250895, 250896, 250897, 250898, 250899, 250900, 250901) c6( ) c7(aw); #c1(TIMD4) c2(NP_001140198) c3(T-cell immunoglobulin and mucin domain-containing protein 4 isoform 2 precursor) c4(60061) c5(60062, 60063, 60064, 60065, 60066, 60067) c6( ) c7(bM, cT, nX, apJ, eZ, y, eh); #c1(TIMD4) c2(NP_612388) c3(T-cell immunoglobulin and mucin domain-containing protein 4 isoform 1 precursor) c4(236335) c5(236336, 236337, 236338, 236339, 236340, 236341) c6( ) c7(bM, cT, nX, apJ, eZ, y, eh); #c1(TIMELESS) c2(NP_003911) c3(protein timeless homolog) c4(142107) c5(142108, 142109, 142110, 142111, 142112, 142113, 142114) c6( ) c7(bM, T, if, Nt, bS, ct, qN, Nv, V, ai, ad, eA, ft, tT, aT, h, alX, bK, ey, oi, uQ, il, aW, hl, eZ, lo, Z, eE, bz, cT, eM, z, fD, bC, ei, hE, B, y, afX); #c1(TIMELESS) c2(XP_011537241) c3 (protein timeless homolog isoform X1) c4(334967) c5(334968, 334969, 334970, 334971, 334972, 334973) c6( ) c7(bM, T, if, Nt, bS, ct, qN, Nv, V, ai, ad, eA, ft, tT, aT, h, alX, bK, ey, oi, uQ, iU, aW, hl, eZ, lo, Z, eE, bz, cT, eM, z, fD, bC, ei, hE, B, y, afX); #c1(TIMM10) c2(NP_036588) c3(mitochondrial import inner membrane translocase subunit Tim10) c4(174645) c5(174646, 174647, 174648, 174649, 174650, 174651, 174652, 174653, 174654, 174655, 174656, 174657, 174658, 174659) c6( ) c7(bv); #c1(TIMM17A) c2(NP_006326) c3(mitochondrial import inner membrane translocase subunit Tim17-A) c4(164048) c5(164049, 164050, 164051, 164052, 164053, 164054) c6( ) c7(aW, kC, aT); #c1(TIMM21) c2(NP_054896) c3(mitochondrial import inner membrane translocase subunit Tim21 precursor) c4(178248) c5(178249, 178250, 178251, 178252, 178253, 178254, 178255, 178256) c6(178257, 178258) c7(W); #c1(TIMM22) c2(NP_037469) c3(mitochondrial import inner membrane translocase subunit Tim22) c4(175678) c5(175679, 175680, 175681, 175682, 175683, 175684) c6( ) c7(aPn); #c1(TIMM23) c2(NP_006318) c3(mitochondrial import inner membrane translocase subunit Tim23) c4(163964, 163965, 163966, 163967, 163968) c5(163969, 163970, 163971, 163972, 163973, 163974, 163975, 163976, 163977, 163978, 163979, 163980, 163981, 163982, 163983, 163984, 163985, 163986, 163987, 163988, 163989, 163990, 163991, 163992, 163993, 163994, 163995, 163996, 163997, 163998) c6( ) c7(afX); #c1(TIMM23) c2(XP_011537400) c3(mitochondrial import inner membrane translocase subunit Tim23 isoform X1) c4(335051) c5( ) c6( ) c7(afX); #c1(TIMM23) c2(XP_011537401) c3 (mitochondrial import inner membrane translocase subunit Tim23 isoform X2) c4(335052) c5(335053, 335054, 335055, 335056, 335057, 335058, 335059, 335060, 335061, 335062, 335063, 335064, 335065, 335066, 335067, 335068, 335069, 335070) c6( ) c7(afX); #c1(TIMM44) c2(NP_006342) c3(mitochondrial import inner membrane translocase subunit TIM44) c4(164176) c5(164177, 164178, 164179, 164180, 164181, 164182) c6( ) c7(aj, hd, J); #c1(TIMM50) c2(NP_001001563) c3(mitochondrial import inner membrane translocase subunit TIM50) c4(18791, 18792) c5(18793, 18794, 18795, 18796, 18797, 18798) c6( ) c7(aW, eM, aT); #c1(TIMM50) c2(XP_011525793) c3(mitochondrial import inner membrane translocase subunit TIM50 isoform X1) c4( ) c5(320198, 320199, 320200, 320201, 320202, 320203) c6( ) c7(aW, eM, aT); #c1(TIMM8A) c2(NP_004076) c3(mitochondrial import inner membrane translocase subunit Tim8 A isoform 1) c4(143360) c5(143361, 143362, 143363, 143364, 143365, 143366) c6( ) c7(T, if, jh, aA, bB, it, vm, cMn, qN, q, eJ, eW, Ks, iu, aE, al, al, dl, eL, aW, Y, r, hQ, aq, aF, bho, t, bz, cm, ik, es, eM, iA, bE, mG, vA, U, m, Bh, B, afX); #c1(TIMM8B) c2(NP_036591) c3(mitochondrial import inner membrane translocase subunit Tim8 B) c4(174660, 174661) c5(174662, 174663, 174664, 174665, 174666, 174667, 174668, 174669, 174670, 174671, 174672, 174673) c6( ) c7(aw); #c1(TIMMDC1) c2(NP_057673) c3(complex I assembly factor TIMMDC1, mitochondrial precursor) c4(192277) c5(192278, 192279, 192280, 192281, 192282, 192283) c6( ) c7(bqS, bc); #c1(TIMP1) c2(NP_003245) c3(metalloproteinase inhibitor 1 precursor) c4(136754, 136755, 136756, 136757) c5(136758, 136759, 136760, 136761, 136762, 136763, 136764, 136765, 136766, 136767, 136768, 136769, 136770, 136771, 136772, 136773, 136774, 136775, 136776, 136777, 136778, 136779, 136780, 136781, 136782, 136783, 136784, 136785, 136786) c6( ) c7(ID, bM, T, aC, fR, pf, ea, if, al, bEy, fd, cMp, bB, cL, aTy, zh, ci, lR, aY, HU, aR, ip, wa, x, Vp, Hr, xE, aE, Ma, ws, fm, K, hu, pk, ass, eL, Ur, cp, ly, yo, up, dH, aW, Rr, Du, hl, uY, bjk, g, as, pL, hJ, uC, eE, eF, mK, bw, aAW, y, fT, eM, nB, iT, wE, mx, vy, tV, nw, k, Hb, cMo, m, fy, R, hh, lS, ym, iu, Ro, ayw, dD, aFG, vl, aRX, aw, NT, qy, n, aj, fr, bd, pM, qN, bkU, cd, ai, ke, afR, Bl, cc, zY, ec, rC, gd, Ak, Lv, h, eV, dL, jl, aUm, ey, hB, aNj, pl, bzr, A, mR, aJ, bMq, fQ, hQ, aq, aGd, fe, pm, mo, CM, sE, avf, lx, KT, W, dB, NL, hk, Pl, Qu, hL, bm, at, uz, B, vQ, bL, Dw, fM, dA, eR, Dh, fG, lH, eX, aA, cz, v, j, C, V, q, J, er, eA, oT, Yn, cfd, qp, ceS, rS, aT, cy, Fx, bq, ae, af, kl, wr, bf, s, De, iU, Y, r, aYp, lo, en, cl, t, pb, ck, nM, ac, c, hc, gX, wq, mf, dy, Mn, JH, fD, Bd, bt, sY, iW, uG, jh, vr, eq, jE, ad, kL, afb, dP, aHF, wD, hd, F, alp, yt, oW, ab, bgr, Aa, nJ, uX, d, cfb, lR, lU, qZ, ff, xs, czY, eH, eZ, yy, dq, Ej, aF, hp, uF, Fq, iV, BG, bz, Hp, LB, D, aOm, gk, bMX, bE, lI, sG, gZ, wz, aS, lf, X, us); #c1(TIMP1) c2(XP_005272702) c3(metalloproteinase inhibitor 1 isoform X1) c4(284152, 284153) c5(284154, 284155, 284156, 284157, 284158, 284159, 284160) c6( ) c7(ID, bM, T, aC, fR, pf, ea, if, al, bEy, fd, cMp, bB, c1, aTy, zh, ci, lR, aY, HU, aR, ip, wa, x, Vp, Hr, xE, aE, Ma, ws, fm, K, hu, pk, ass, eL, Ur, cp, ly, yo, up, dH, aW, Rr, Du, hl, uY, bjk, g, as, pl, hJ, uC, eE, eF, mK, bw, aAW, y, fT, eM, nB, iT, wE, mx, vy, tV, nw, k, Hb, cMo, m, fy, R, hh, lS, ym, iu, Ro, ayw, dD, aFG, vl, aRX, aw, NT, qy, n, aj, fr, bd, pM, qN, bkU, cd, ai, ke, afR, Bl, cc, zY, ec, rC, gd, Ak, Lv, h, eV, dL, jl, aUm, ey, hB, aNj, pl, bzr, A, mR, aJ, bMq, fQ, hQ, aq, aGd, fe, pm, mo, cM, sE, avf, lx, KT, W, dB, NL, hk, Pl, Qu, hl, bm, at, uz, B, vQ, bL, Dw, fM, dA, eR, Dh, fG, lH, eX, aA, cz, v, j, C, V, q, J, er, eA, oT, Yn, cfd, qp, ceS, rS, aT, cy, Fx, bq, ae, af, kl, wr, bf, s, De, iU, Y, r, aYp, lo, en, cl, t, pb, ck, nM, ac, c, hc, gX, wq, mf, dy, Mn, JH, fD, Bd, bt, sY, iW, uG, jh, vr, eq, jE, ad, kL, afb, dP, aHF, wD, hd, F, alp, yt, oW, ab, bgr, Aa, nJ, uX, d, cfb, lR, lU, qZ, ff, xs, czY, eH, eZ, yy, dq, Ej, aF, hp, uF, Fq, iV, BG, bz, Hp, LB, D, aOm, gk, bMX, bE, lI, sG, gZ, wz, aS, lf, X, us); #c1(TIMP2) c2(NP_003246) c3(metalloproteinase inhibitor 2 precursor) c4(136787, 136788) c5(136789, 136790, 136791, 136792, 136793, 136794, 136795, 136796, 136797, 136798, 136799, 136800) c6( ) c7(T, aC, if, al, bEy, fd, bB, ci, Tz, ip, x, Na, dY, aE, M, eL, DA, eG, Jl, aW, uY, g, as, eF, cm, uC, bw, aAW, y, fT, eM, ki, vA, tV, k, m, gn, fy, Vj, iu, dD, aFG, vl, FG, aO, aw, NT, n, aj, fr, bd, qN, cd, ai, cc, Ak, h, eV, dL, jl, hB, cY, bzr, aJ, hQ, fe, aq, cM, sE, op, dB, hk, wk, Be, hL, at, uz, bm, Pr, B, ka, fM, dA, eR, mr, wL, eX, aA, cz, j, C, V, Eh, q, J, eA, qp, aT, Nc, bq, ae, af, bA, bf, il, De, iZ, Ue, Y, r, uU, en, cl, bl, t, nC, ik, pb, ck, ajl, rR, ac, c, dy, Gg, JH, bt, JO, hS, sY, iW, uG, jh, vr, eq, jE, ad, afb, wD, dP, hd, F, sN, io, ab, vc, nJ, WZ, mu, uX, xx, cfb, d, ff, xs, NH, eH, Ej, aF, bz, bE, azT, sG, aS, lf, X, us); #c1(TIMP3) c2(NP_000353) c3(metalloproteinase inhibitor 3 precursor) c4(7810, 7811) c5(7812, 7813, 7814, 7815, 7816, 7817, 7818, 7819, 7820, 7821, 7822, 7823, 7824) c6( ) c7(bM, T, fR, pf, ea, if, al, Xi, eb, bB, zh, ci, lR, Hr, ip, Na, dY, K, eL, cMq, aW, hl, g, Uk, iP, ba, eM, mx, bjY, U, aqV, uo, vy, nw, m, bJv, de, rt, cZ, dD, vl, sQ, tp, aw, n, aj, fr, qN, PJ, rP, cd, jz, lG, bci, dl, dd, db, A, qY, fe, aq, kp, W, Be, bm, uz, rx, ut, sZ, B, it, bL, dA, fG, aA, cz, v, j, AO, V, q, J, eA, BJ, aT, Nc, Fx, ae, jf, bf, s, Y, bg, r, en, cl, t, nC, btO, Dm, pb, iK, nM, ac, c, dh, aJQ, np, dy, bnl, vi, bGg, iW, vr, eq, ad, av, hd, F, ard, py, Sr, ab, Aa, aqX, jo, lQ, aNR, ji, nJ, uX, d, lR, qt, ff, xs, cr, aF, bz, ayj, iq, LB, es, TA, amS, nb, bE, aS, be, GF, y, us); #c1(TIMP4) c2(NP_003247) c3(metalloproteinase inhibitor 4 precursor) c4(136801) c5(136802, 136803, 136804, 136805, 136806, 136807) c6( ) c7(bL, T, fR, sQ, uv, al, jx, aw, aj, j, uE, aCQ, Fc, cd, J, ad, ai, er, eA, hd, F, Bc, aT, h, cy, Fx, K, lQ, ae, Dl, d, De, aW, fO, as, pl, pb, Hp, LB, D, nB, dc, lf, bm, tV, uo, uz, ut, fy, B, y); #c1(TINAG) c2(NP_055279) c3(tubulointerstitial nephritis antigen) c4(180244, 180245) c5(180246, 180247, 180248, 180249, 180250, 180251, 180252) c6( ) c7(dy); #c1(TINAG) c2(XP_006715124) c3(tubulointerstitial nephritis antigen isoform X1) c4(288752) c5( ) c6( ) c7(dy); #c1(TINAG) c2(XP_006715126) c3(tubulointerstitial nephritis antigen isoform X3) c4( ) c5(288753, 288754, 288755, 288756, 288757, 288758, 288759, 288760, 288761, 288762, 288763) c6( ) c7(dy); #c1(TINAG) c2(XP_011512801) c3(tubulointerstitial nephritis antigen isoform X7) c4(303860, 303861) c5(303862, 303863, 303864, 303865, 303866, 303867, 303868, 303869, 303870, 303871, 303872, 303873) c6( ) c7(dy); #c1(TINF2) c2(NP_001092744) c3(TERF1-interacting nuclear factor 2 isoform 1) c4(39605) c5(39606, 39607, 39608, 39609, 39610, 39611, 39612) c6( ) c7(aA, ci, cMs, auf, nX, kH, cMr, pl, sb, Ng, je, byn, Y, cKZ, iM, aWZ, ajw, dW, aiS, oX, xb, aYd); #c1(TINF2) c2(NP_036593) c3(TERF1-interacting nuclear factor 2 isoform 2) c4(174674) c5(174675, 174676, 174677, 174678, 174679, 174680, 174681, 174682, 174683, 174684) c6( ) c7(aA, ci, cMs, auf, nX, kH, cMr, pl, sb, Ng, je, byn, Y, cKZ, iM, aWZ, ajw, dW, aiS, oX, xb, aYd); #c1(TINF2) c2(XP_005267586) c3(TERF1-interacting nuclear factor 2 isoform X2) c4(281024, 281025) c5(281026, 281027, 281028, 281029, 281030, 281031, 281032) c6( ) c7(aA, ci, cMs, auf, nX, kH, cMr, pl, sb, Ng, je, byn, Y, cKZ, iM, aWZ, ajw, dW, aiS, oX, xb, aYd); #c1(TIPARP) c2(NP_001171646) c3(TCDD-inducible poly [ADP-ribose] polymerase) c4(71107, 71108, 71109) c5(71110, 71111, 71112, 71113, 71114, 71115, 71116, 71117, 71118, 71119, 71120, 71121, 71122, 71123, 71124, 71125, 71126, 71127, 71128, 71129, 71130, 71131, 71132, 71133, 71134, 71135, 71136, 71137, 71138, 71139) c6( ) c7(bM, T, aw, aA, Hn, aj, qN, q, ad, eA, hX, eW, hd, aE, aT, h, cy, K, NM, ae, dx, cp, d, B, s, aW, Y, Yd, eH, ff, r, aF, hp, en, eF, t, cm, axG, bF, ac, c, nB, bE, m, hE, cnU, y); #c1(TIPIN) c2(NP_001276915) c3(TIMELESS-interacting protein isoform 2) c4( ) c5(108618, 108619, 108620, 108621, 108622) c6( ) c7(K); #c1(TIPIN) c2(NP_060328) c3(TIMELESS-interacting protein isoform 1) c4(195412) c5( ) c6( ) c7(K); #c1(TIPIN) c2(XP_005254578) c3(TIMELESS-interacting protein isoform X1) c4(273257) c5(273258, 273259, 273260, 273261, 273262, 273263, 273264) c6( ) c7(K); #c1(TIPRL) c2(NP_001026970) c3(TIP41-like protein isoform 2) c4(29998) c5(29999, 30000, 30001, 30002, 30003, 30004) c6( ) c7(B, bA, cz); #c1(TIPRL) c2(NP_690866) c3(TIP41-like protein isoform 1) c4(245019) c5(245020, 245021, 245022, 245023, 245024, 245025) c6( ) c7(B, bA, cz); #c1(TIRAP) c2(XP_005271456) c3(toll/interleukin-1 receptor domain-containing adapter protein isoform X3) c4( ) c5(283424, 283425, 283426, 283427, 283428) c6( ) c7(oO, T, fR, azW, aHV, if, ht, ex, aA, fF, BH, v, ci, C, eg, pU, q, ad, akU, Uh, dV, Os, aT, h, eV, bTf, iY, aaW, hu, lZ, aW, Zf, eZ, r, fe, aq, ls, af, uF, aCK, eE, t, f, ik, ck, cMt, ig, ki, gX, Br, aYx, m, EY, fD, y, bt, B, aDj); #c1(TIRAP) c2(XP_011540883) c3(toll/interleukin-1 receptor domain-containing adapter protein isoform X2) c4(339402) c5(339403, 339404, 339405, 339406, 339407) c6( ) c7(oO, T, fR, azW, aHV, if, ht, eX, aA, fF, BH, v, ci, C, eg, pl, q, ad, akU, Uh, dV, Os, aT, h, eV, bTf, iY, aaW, hu, lZ, aW, Zf, eZ, r, fe, aq, ls, aF, uF, aCK, eE, t, f, ik, ck, cMt, ig, ki, gX, Br, aYx, m, EY, fD, y, bt, B, aDj); #c1(TIRAP) c2(XP_011540884) c3(toll/interleukin-1 receptor domain-containing adapter protein isoform X1) c4(339408, 339409) c5(339410, 339411, 339412, 339413, 339414, 339415, 339416, 339417, 339418, 339419, 339420, 339421, 339422, 339423, 339424, 339425) c6( ) c7(oO, T, fR, azW, aHV, if, ht, eX, aA, fF, BH, v, ci, C, eg, pU, q, ad, akU, Uh, dV, Os, aT, h, eV, bTf, iY, aaW, hu, lZ, aW, Zf, eZ, r, fe, aq, ls, af, uF, aCK, eE, t, f, ik, ck, cMt, ig, ki, gX, Br, aYx, m, EY, fD, y, bt, B, aDj); #c1(TJP1) c2(NP_003248) c3(tight junction protein ZO-1 isoform a) c4(136808) c5( ) c6( ) c7(bM, atj, RX, jx, qW, cd, Bl, hb, zf, aT, lG, bq, dF, eu, A, aW, Y, fO, hp, hJ, nB, agl, wz, aS, B); #c1(TJP1) c2(NP_783297) c3(tight junction protein ZO-1 isoform b) c4(251579) c5( ) c6( ) c7(bM, atj, RX, jx, qW, cd, Bl, hb, zf, aT, lG, bq, dF, eu, A, aW, Y, fO, hp, hJ, nB, agl, wz, aS, B); #c1(TJP1) c2(XP_006725617) c3(tight junction protein ZO-1 isoform X2) c4( ) c5(296925, 296926, 296927, 296928, 296929, 296930) c6( ) c7(bM, atj, RX, jx, qW, cd, Bl, hb, zf, aT, lG, bq, dF, eu, A, aW, Y, fO, hp, hJ, nB, agl, wz, aS, B); #c1(TJP1) c2(XP_006725621) c3(tight junction protein ZO-1 isoform X9) c4( ) c5(296931, 296932, 296933, 296934, 296935, 296936) c6( ) c7(bM, atj, RX, jx, qW, cd, Bl, hb, zf, aT, lG, bq, dF, eu, A, aW, Y, fO, hp, hJ, nB, agl, wz, aS, B); #c1(TJP2) c2(NP_001163885) c3(tight junction protein ZO-2 isoform 5) c4(66467) c5( ) c6( ) c7(Bl, cl, eJ, AO, qt, Y, cz, cl, v, ci, jw, Bh, anh, o, wT); #c1(TJP2) c2(NP_001163886) c3(tight junction protein ZO-2 isoform 4) c4(66468) c5(66469, 66470, 66471, 66472, 66473, 66474) c6( ) c7(Bl, cl, eJ, AO, qt, Y, cz, cl, v, ci, jw, Bh, anh, o, wT); #c1(TJP2) c2(NP_001163887) c3 (tight junction protein ZO-2 isoform 3) c4(66475) c5(66476, 66477, 66478, 66479, 66480, 66481) c6( ) c7(Bl, cl, eJ, AO, qt, Y, cz, cl, v, ci, jw, Bh, anh, o, wT); #c1(TJP2) c2(NP_001164101) c3(tight junction protein ZO-2 isoform 6) c4(66603) c5(66604, 66605, 66606, 66607, 66608, 66609, 66610, 66611, 66612, 66613, 66614, 66615, 66616, 66617, 66618, 66619, 66620) c6( ) c7(Bl, cl, eJ, AO, qt, Y, cz, cl, v, ci, jw, Bh, anh, o, wT); #c1(TJP2) c2(NP_004808) c3(tight junction protein ZO-2 isoform 1) c4(149980) c5(149981, 149982, 149983, 149984, 149985, 149986) c6( ) c7(Bl, cl, eJ, AO, qt, Y, cz, cl, v, ci, jw, Bh, anh, o, wT); #c1(TJP2) c2(NP_963923) c3(tight junction protein ZO-2 isoform 2) c4(262564) c5(262565, 262566, 262567, 262568, 262569, 262570, 262571, 262572, 262573, 262574, 262575, 262576) c6( ) c7(Bl, cl, eJ, AO, qt, Y, cz, cl, v, ci, jw, Bh, anh, o, wT); #c1(TJP2) c2(XP_006717387) c3(tight junction protein ZO-2 isoform X3) c4( ) c5(291042) c6( ) c7(Bl, cl, eJ, AO, qt, Y, cz, cl, v, ci, jw, Bh, anh, o, wT); #c1(TJP2) c2(XP_011517511) c3(tight junction protein ZO-2 isoform X4) c4(311304, 311305) c5( ) c6( ) c7(Bl, cl, eJ, AO, qt, Y, cz, cl, v, ci, jw, Bh, anh, o, wT); #c1(TJP3) c2(NP_001254489) c3(tight junction protein ZO-3 isoform 1) c4(94484) c5( ) c6( ) c7(ad, MD); #c1(TJP3) c2(NP_001254490) c3(tight junction protein ZO-3 isoform 2) c4(94485, 94486, 94487, 94488) c5(94489, 94490, 94491, 94492, 94493, 94494, 94495, 94496, 94497, 94498, 94499, 94500, 94501, 94502, 94503, 94504, 94505, 94506, 94507, 94508, 94509, 94510, 94511, 94512, 94513, 94514, 94515, 94516, 94517, 94518) c6( ) c7(ad, MD); #c1(TK1) c2(NP_003249) c3(thymidine kinase, cytosolic) c4(136809) c5(136810, 136811, 136812, 136813, 136814, 136815, 136816, 136817, 136818, 136819) c6( ) c7(T, fM, jp, qx, aDT, Hn, ci, eA, aoz, eW, av, aE, oj, aT, h, btE, fb, vT, aW, t, bw, btK, eM, dB, cj, hR, bt); #c1(TK2) c2(NP_001166114) c3(thymidine kinase 2 isoform 2) c4(68201, 68202) c5(68203, 68204, 68205, 68206, 68207, 68208, 68209, 68210) c6( ) c7(qx, ha, Hn, aNG, ci, bgR, aqQ, aoz, oj, kf, yZ, btE, fb, cJF, vT, aqP, px, bw, btk, dR, ac, ky, KB, U, awl); #c1(TK2) c2(NP_001166115) c3(thymidine kinase 2, mitochondrial isoform 3 precursor) c4(68211) c5(68212, 68213, 68214, 68215, 68216, 68217) c6( ) c7(qx, ha, Hn, aNG, ci, bgR, aql, aoz, oj, kf, yZ, btE, fb, cJF, vT, aqP, px, bw, btk, dR, ac, ky, kB, U, awl); #c1(TK2) c2(NP_001166116) c3 (thymidine kinase 2, mitochondrial isoform 4 precursor) c4(68218) c5(68219, 68220, 68221, 68222, 68223, 68224) c6( ) c7(qx, ha, Hn, aNG, ci, bgR, aql, aoz, oj, kf, yZ, btE, fb, cJF, vT, aqP, px, bw, btK, dR, ac, ky, kB, U, awl); #c1(TK2) c2(NP_001258863) c3(thymidine kinase 2 isoform 5) c4(96598) c5(96599, 96600, 96601, 96602, 96603, 96604) c6( ) c7(qx, ha, Hn, aNG, ci, bgR, aqQ, aoz, oj, kf, yZ, btE, fb, cJF, vT, aqP, px, bw, btk, dR, ac, ky, kB, U, awl); #c1(TK2) c2(NP_001258979) c3(thymidine kinase 2, mitochondrial isoform 7 precursor) c4( ) c5(96892, 96893, 96894, 96895, 96896, 96897, 96898, 96899) c6( ) c7(qx, ha, Hn, aNG, ci, bgR, aqQ, aoz, oj, kf, yZ, btE, fb, cJF, vT, aqP, px, bw, btK, dR, ac, ky, kB, U, awl); #c1(TK2) c2(NP_004605) c3(thymidine kinase 2, mitochondrial isoform 1 precursor) c4(148090, 148091) c5(148092, 148093, 148094, 148095, 148096, 148097, 148098, 148099, 148100, 148101, 148102, 148103) c6( ) c7(qx, ha, Hn, aNG, ci, bgR, aqQ, aoz, oj, kf, yZ, btE, fb, cJF, vT, aqP, px, bw, btk, dR, ac, ky, kB, U, awl); #c1(TKT) c2(NP_001128527) c3(transketolase isoform 1) c4(50181, 50182) c5(50183, 50184, 50185, 50186, 50187, 50188, 50189, 50190, 50191, 50192, 50193, 50194, 50195, 50196, 50197, 50198, 50199) c6( ) c7(T, abY, cCU, if, aj, dG, ci, qN, zP, V, J, Gm, mj, yt, Xe, ajU, zQ, iU, aq, aqM, Gs, U, B); #c1(TKT) c2(NP_001244957) c3(transketolase isoform 2) c4(92444) c5(92445, 92446, 92447, 92448, 92449, 92450) c6( ) c7(T, abY, cCU, if, aj, dG, ci, qN, zP, V, J, Gm, mj, yt, Xe, ajU, zQ, il, aq, aqM, Gs, U, B); #c1(TKT) c2(XP_011532357) c3(transketolase isoform X2) c4(328512) c5(328513, 328514, 328515, 328516, 328517) c6( ) c7(T, abY, cCU, if, aj, dG, ci, qN, zP, V, J, Gm, mj, yt, Xe, ajU, zQ, iU, aq, aqM, Gs, U, B); #c1(TKTL1) c2(NP_001139405) c3(transketolase-like protein 1 isoform b) c4(59287, 59288) c5(59289) c6( ) c7(T, if, jh, aA, OR, oH, ci, qN, q, hd, BJ, aT, btE, K, fb, d, iU, vT, aW, Y, ff, r, aq, aF, en, ik, op, btk, nM, nB, vA, B, Ex); #c1(TKTL1) c2(NP_001139406) c3(transketolase-like protein 1 isoform c) c4( ) c5(59290, 59291, 59292, 59293, 59294, 59295, 59296, 59297, 59298, 59299, 59300, 59301, 59302, 59303, 59304, 59305) c6( ) c7(T, if, jh, aA, OR, oH, ci, qN, q, hd, BJ, aT, btE, K, fb, d, iU, vT, aW, Y, ff, r, aq, aF, en, ik, op, btK, nM, nB, vA, B, Ex); #c1(TKTL1) c2(NP_036385) c3(transketolase-like protein 1 isoform a) c4(173455, 173456) c5(173457, 173458, 173459, 173460, 173461, 173462, 173463, 173464, 173465, 173466) c6( ) c7(T, if, jh, aA, OR, oH, ci, qN, q, hd, BJ, aT, btE, K, fb, d, il, vT, aW, Y, ff, r, aq, aF, en, ik, op, btK, nM, nB, vA, B, Ex); #c1(TKTL2) c2(NP_115512) c3(transketolase-like protein 2) c4(223011) c5(223012, 223013, 223014, 223015, 223016, 223017) c6( ) c7(if, oH, qN); #c1(TLDC1) c2(XP_005256131) c3(TLD domain-containing protein 1 isoform X2) c4(273976) c5(273977) c6( ) c7(akY); #c1(TLDC1) c2(XP_005256132) c3(TLD domain-containing protein 1 isoform X1) c4(273978, 273979) c5(273980, 273981, 273982, 273983, 273984, 273985, 273986, 273987, 273988, 273989, 273990, 273991, 273992, 273993, 273994, 273995, 273996) c6( ) c7(akY); #c1(TLE1) c2(NP_001290032) c3(transducin-like enhancer protein 1 isoform 1) c4( ) c5(114941) c6( ) c7(bL, ck, xN, T, il, QG, aW, mg, r, aT, bS, hP, ct, iJ, Z, bt, bz, iu); #c1(TLE1) c2(NP_001290033) c3(transducin-like enhancer protein 1 isoform 3) c4( ) c5(114942, 114943, 114944, 114945, 114946, 114947) c6( ) c7(bL, ck, xN, T, il, QG, aW, mg, r, aT, bS, hP, ct, iJ, Z, bt, bz, iu); #c1(TLE1) c2(NP_005068) c3(transducin-like enhancer protein 1 isoform 2) c4(152580) c5(152581, 152582, 152583, 152584, 152585, 152586, 152587) c6( ) c7(bL, ck, xN, T, il, QG, aW, mg, r, aT, bS, hP, ct, iJ, Z, bt, bz, iu); #c1(TLE1) c2(XP_005252210) c3(transducin-like enhancer protein 1 isoform X4) c4(271609) c5( ) c6( ) c7(bL, ck, xN, T, il, QG, aW, mg, r, aT, bS, hP, ct, iJ, Z, bt, bz, iu); #c1(TLE1) c2(XP_005252213) c3(transducin-like enhancer protein 1 isoform X6) c4(271610, 271611) c5(271612, 271613, 271614, 271615, 271616, 271617, 271618, 271619, 271620, 271621, 271622) c6( ) c7(bL, ck, xN, T, il, QG, aW, mg, r, aT, bS, hP, ct, iJ, Z, bt, bz, iu); #c1(TLE1) c2(XP_005252220) c3(transducin-like enhancer protein 1 isoform X12) c4(271623) c5(271624, 271625, 271626, 271627, 271628, 271629, 271630, 271631, 271632) c6( ) c7(bL, ck, xN, T, il, QG, aW, mg, r, aT, bS, hP, ct, iJ, Z, bt, bz, iu); #c1(TLE2) c2(NP_001138233) c3(transducin-like enhancer protein 2 isoform 2) c4(56089, 56090) c5(56091, 56092, 56093, 56094, 56095, 56096, 56097, 56098, 56099, 56100, 56101, 56102, 56103, 56104, 56105, 56106) c6( ) c7(as, bEu, ip); #c1(TLE2) c2(NP_001138234) c3(transducin-like enhancer protein 2 isoform 3) c4(56107) c5(56108, 56109, 56110, 56111, 56112, 56113, 56114, 56115, 56116, 56117, 56118, 56119, 56120, 56121, 56122, 56123, 56124, 56125, 56126) c6( ) c7(as, bEu, ip); #c1(TLE2) c2(NP_001287775) c3(transducin-like enhancer protein 2 isoform 4) c4(112855) c5(112856) c6( ) c7(as, bEu, ip); #c1(TLE2) c2(NP_003251) c3(transducin-like enhancer protein 2 isoform 1) c4(136827, 136828) c5(136829, 136830, 136831, 136832, 136833, 136834) c6( ) c7(as, bEu, ip); #c1(TLE2) c2(XP_005259694) c3(transducin-like enhancer protein 2 isoform X3) c4(276231) c5(276232, 276233, 276234, 276235, 276236, 276237, 276238, 276239) c6( ) c7(as, bEu, ip); #c1(TLE3) c2(NP_001098662) c3(transducin-like enhancer protein 3 isoform b) c4(41375) c5(41376, 41377, 41378, 41379, 41380, 41381, 41382, 41383, 41384, 41385, 41386, 41387) c6( ) c7(aT, bk, PJ, y, ad, ip); #c1(TLE3) c2(NP_001269908) c3(transducin-like enhancer protein 3 isoform e) c4(103124, 103125) c5(103126, 103127, 103128, 103129, 103130, 103131, 103132, 103133, 103134, 103135, 103136, 103137) c6( ) c7(aT, bK, PJ, y, ad, ip); #c1(TLE3) c2(NP_001269909) c3(transducin-like enhancer protein 3 isoform d) c4(103138) c5(103139, 103140, 103141, 103142, 103143, 103144) c6( ) c7(aT, bK, PJ, y, ad, ip); #c1(TLE3) c2(NP_001269910) c3(transducin-like enhancer protein 3 isoform f) c4(103145, 103146) c5(103147, 103148, 103149, 103150, 103151, 103152) c6( ) c7(aT, bk, PJ, y, ad, ip); #c1(TLE3) c2(NP_001269911) c3(transducin-like enhancer protein 3 isoform g) c4( ) c5(103153, 103154) c6( ) c7(aT, bK, PJ, y, ad, ip); #c1(TLE3) c2(NP_005069) c3(transducin-like enhancer protein 3 isoform a) c4(152588) c5(152589, 152590, 152591, 152592, 152593, 152594) c6( ) c7(aT, bK, PJ, y, ad, ip); #c1(TLE3) c2(NP_065959) c3(transducin-like enhancer protein 3 isoform c) c4(206485) c5(206486, 206487, 206488, 206489, 206490, 206491) c6( ) c7(aT, bK, PJ, y, ad, ip); #c1(TLE4) c2(NP_001269677) c3(transducin-like enhancer protein 4 isoform 1) c4(102510, 102511) c5(102512, 102513, 102514, 102515, 102516, 102517, 102518, 102519, 102520, 102521, 102522, 102523) c6( ) c7(bM, zQ, s, aW, EQ, aT, DE, v, gv, ci, bK, j, zP, bt); #c1(TLE4) c2(NP_001269678) c3(transducin-like enhancer protein 4 isoform 2) c4(102524) c5(102525, 102526, 102527, 102528, 102529, 102530) c6( ) c7(bM, zQ, s, aW, EQ, aT, OE, v, gv, ci, bK, j, zP, bt); #c1(TLE4) c2(NP_001269682) c3(transducin-like enhancer protein 4 isoform 4) c4(102536) c5(102537, 102538, 102539, 102540, 102541, 102542) c6( ) c7(bM, zQ, s, aW, EQ, aT, OE, v, gv, ci, bK, j, zP, bt); #c1(TLE4) c2(NP_001269689) c3(transducin-like enhancer protein 4 isoform 5) c4( ) c5(102569, 102570, 102571, 102572, 102573) c6( ) c7(bM, zQ, s, aW, EQ, aT, OE, v, gv, ci, bK, j, zP, bt); #c1(TLE4) c2(NP_008936) c3(transducin-like enhancer protein 4 isoform 3) c4(169654) c5(169655, 169656, 169657, 169658, 169659, 169660, 169661) c6( ) c7(bM, zQ, s, aW, EQ, aT, DE, v, gv, ci, bK, j, zP, bt); #c1(TLE4) c2(XP_0067 (7331) c3(transducin-like enhancer protein 4 isoform X11) c4(290920) c5( ) c6( ) c7(bM, zQ, s, aW, EQ, aT, DE, v, gv, ci, bK, j, zP, bt); #c1(TLE4) c2(XP_011517258) c3(transducin-like enhancer protein 4 isoform X6) c4(310859) c5(310860, 310861, 310862, 310863, 310864, 310865) c6( ) c7(bM, zQ, s, aW, EQ, aT, OE, v, gv, ci, bK, j, zP, bt); #c1(TLE6) c2(NP_001137458) c3(transducin-like enhancer protein 6 isoform 1) c4(55833, 55834, 55835, 55836) c5(55837, 55838, 55839, 55840, 55841, 55842, 55843, 55844, 55845, 55846, 55847, 55848, 55849, 55850) c6( ) c7(X, gX); #c1(TLE6) c2(NP_079036) c3(transducin-like enhancer protein 6 isoform 2) c4( ) c5(216699, 216700, 216701, 216702, 216703, 216704, 216705, 216706, 216707, 216708) c6( ) c7(X, gX); #c1(TLK1) c2(NP_001130026) c3(serine/threonine-protein kinase tousled-like 1 isoform 2) c4(52443) c5(52444, 52445, 52446, 52447, 52448, 52449, 52450, 52451) c6( ) c7(eW, T, jz, Y, qK, aT, alJ, gv, K, ey, MC, ae, ne, ad); #c1(TLK1) c2(NP_001130027) c3(serine/threonine-protein kinase tousled-like 1 isoform 3) c4(52452) c5(52453, 52454, 52455, 52456, 52457, 52458) c6( ) c7(eW, T, jz, Y, qK, aT, alJ, gv, K, ey, MC, ae, ne, ad); #c1(TLK1) c2(NP_036422) c3(serine/threonine-protein kinase tousled-like 1 isoform 1) c4(173698, 173699, 173700) c5(173701, 173702, 173703, 173704, 173705, 173706, 173707, 173708, 173709, 173710, 173711, 173712, 173713, 173714) c6( ) c7(eW, T, jz, Y, qK, aT, alJ, gv, K, ey, MC, ae, ne, ad); #c1(TLK1) c2(XP_005247038) c3(serine/threonine-protein kinase tousled-like 1 isoform X2) c4( ) c5(267593, 267594, 267595, 267596, 267597, 267598) c6( ) c7(eW, T, jz, Y, qK, aT, alJ, gv, K, ey, MC, ae, ne, ad); #c1(TLK2) c2(NP_001271262) c3(serine/threonine-protein kinase tousled-like 2 isoform C) c4(103772) c5(103773, 103774, 103775, 103776, 103777, 103778) c6( ) c7(aW, al, aq, cm, ae, B, ad); #c1(TLK2) c2(NP_001271292) c3(serine/threonine-protein kinase tousled-like 2 isoform B) c4(103856) c5(103857, 103858, 103859, 103860, 103861, 103862, 103863, 103864, 103865, 103866) c6( ) c7(aW, aT, aq, cm, ae, B, ad); #c1(TLK2) c2(NP_006843) c3(serine/threonine-protein kinase tousled-like 2 isoform A) c4(168591) c5(168592, 168593, 168594, 168595, 168596, 168597) c6( ) c7(aW, aT, aq, cm, ae, B, ad); #c1(TLL1) c2(NP_001191689) c3(tolloid-like protein 1 isoform 2 precursor) c4(81613) c5(81614, 81615, 81616, 81617, 81618, 81619) c6( ) c7(bL, nD, cMu, oe, eH, j, zp, eF, cd); #c1(TLL1) c2(NP_036596) c3(tolloid-like protein 1 isoform 1 precursor) c4(174692) c5(174693, 174694, 174695, 174696, 174697, 174698, 174699, 174700) c6( ) c7(bL, nD, cMu, De, eH, j, zp, eF, cd); #c1(TLL2) c2(NP_036597) c3(tolloid-like protein 2 precursor) c4(174701) c5(174702, 174703, 174704, 174705, 174706, 174707) c6( ) c7(bK); #c1(TLN1) c2(NP_006280) c3(talin-1) c4(163525, 163526) c5(163527, 163528, 163529, 163530, 163531, 163532, 163533, 163534, 163535, 163536, 163537, 163538) c6( ) c7(T, kP, yt, aW, ff, aT, n, K, en, ae, B, ad, bA); #c1(TLN2) c2(XP_011520408) c3(talin-2 isoform X1) c4(315281) c5(315282, 315283, 315284, 315285, 315286, 315287, 315288, 315289, 315290, 315291) c6( ) c7(il, bX); #c1(TLR1) c2(NP_003254) c3(toll-like receptor 1 precursor) c4(136836, 136837, 136838, 136839, 136840) c5(136841, 136842, 136843, 136844, 136845, 136846, 136847, 136848, 136849, 136850, 136851, 136852, 136853, 136854, 136855, 136856, 136857, 136858, 136859, 136860, 136861, 136862, 136863, 136864, 136865, 136866, 136867, 136868, 136869, 136870, 136871, 136872, 136873, 136874) c6( ) c7(bM, T, FR, fd, ci, ip, wa, azY, AW, cMy, dV, Ds, cp, aW, az, md, g, eE, bw, uC, eM, cMv, m, eg, aNn, aSs, xN, fr, aBl, ai, Uh, h, ey, aDp, cMw, fe, aq, nN, aOL, aDE, it, B, cMz, Dw, DM, dA, aZy, aA, j, acn, C, V, q, aOj, aWc, akU, jZ, sl, aT, ae, dl, Y, Pg, r, aDm, en, cl, t, Ym, cMx, nM, ig, wR, CZ, EY, aDj, sY, azW, iW, jh, ad, oW, BO, ff, aZw, afr, aF, uF, Sm, iR, iV, wz, gy, y); #c1(TLR10) c2(XP_011512064) c3(toll-like receptor 10 isoform X1) c4(302701) c5(302702, 302703, 302704, 302705, 302706, 302707, 302708, 302709, 302710, 302711, 302712, 302713, 302714, 302715, 302716, 302717, 302718, 302719) c6( ) c7(bM, Dw, nM, aW, sp, iW, ml, ja, aT, fd, Ds, V, ae, ad, wa); #c1(TLR2) c2(XP_011530518) c3(toll-like receptor 2 isoform X1) c4(326441) c5(326442, 326443, 326444, 326445, 326446, 326447) c6( ) c7(oO, bM, T, lD, aC, xS, fR, pf, ea, if, e, qx, al, eb, bB, fd, qj, cMF, bRn, ci, Tz, wa, aEQ, bPE, AW, cMy, aE, Bc, M, dV, Ds, azm, bnF, K, aZS, zy, hH, DY, bc, Us, hm, bzH, bJX, ly, aa, aEV, LC, aW, hC, bNq, cMA, az, hl, md, ih, kt, pD, zC, aB, pl, eE, axp, uC, bw, zV, bm, aEP, fT, eM, aDs, xD, dc, ki, cj, uo, Zn, uJ, bas, Hb, m, R, aNn, eg, jM, abh, blB, ajy, dD, aBe, QO, sQ, Ht, aw, xM, aj, BF, aBl, pM, qN, Za, eh, zo, ai, eW, aDF, Vs, baA, cc, wG, Uh, eT, h, lG, jy, dL, bOp, aFi, ux, hB, wb, aDp, ey, Nr, asp, A, oo, sp, EU, fQ, aq, po, fe, CA, nN, ajz, jT, cM, bxB, lx, OU, Wt, cpN, KT, ar, cMC, dB, bOj, bRS, mg, aOL, hl, bPs, ut, uz, eo, at, aDE, JM, B, vQ, bL, cMz, Dw, lk, AL, cMG, eR, fG, jx, YN, aee, cR, aA, cz, hy, j, v, C, aBo, azF, V, q, J, eA, DK, aWc, jZ, sl, qp, aT, Di, cy, cMB, bK, cMH, yb, bq, aAt, qG, af, dl, aFs, le, bNR, aQk, xR, cMe, Y, fO, Pg, r, iy, akY, lo, ls, afq, t, Ym, pb, aff, ajl, cT, ac, ald, ig, WP, cmU, fP, wq, wR, ro, cS, dy, CMD, EY, JH, fD, bt, mf, CZ, aDj, ao, HQ, sY, hl, BD, Ke, azW, akd, LX, aD, df, axd, iW, jh, vr, dG, eq, jE, atK, ad, aoH, LH, hX, F, bNN, aFl, ds, Os, rN, io, vB, aNp, lQ, jo, ji, XI, NM, vO, cME, ul, CL, BN, anX, Zf, cr, eZ, dN, aFr, aF, si, aDd, hp, aCK, aFd, uF, iV, aDH, Nq, LB, gk, axs, bE, z, anY, wz, sG, gy, ei, lf, ra, ne, y, pC); #c1(TLR3) c2(NP_003256) c3(toll-like receptor 3 precursor) c4(136875, 136876, 136877) c5(136878, 136879, 136880, 136881, 136882, 136883, 136884, 136885, 136886, 136887, 136888, 136889, 136890, 136891, 136892, 136893, 136894, 136895) c6( ) c7(oO, bM, T, xS, fR, pf, e, bB, ci, bh, cMl, fw, wa, xy, aE, aDk, Ds, K, aDU, bc, aa, bkr, aW, bNq, hl, apn, md, g, cm, uC, Uk, aDX, nB, eM, mx, uo, nw, jM, hh, cZ, sQ, NT, fN, xM, n, fr, ct, uE, aBl, Za, ai, eW, cc, cU, lG, h, aPe, ey, eV, dd, aDp, afu, bvO, A, aXy, ZC, lb, fe, aq, pm, jT, ar, W, aCz, NL, hL, ut, fL, B, Dw, fG, jx, eX, cz, fF, v, C, bX, V, q, hb, akl, aT, ae, Y, fO, r, akY, lo, en, bNt, nM, ac, hc, AH, wR, fD, bt, sY, BD, jh, wV, bS, jE, mC, ad, hX, dP, F, ds, akJ, nl, oW, ji, lQ, d, qZ, ff, NH, nh, eZ, hp, Fq, Z, aDH, LB, Bv, bE, Lj, wz, Xs, GF, y); #c1(TLR4) c2(NP_003257) c3(toll-like receptor 4 isoform C) c4(136896) c5(136897, 136898, 136899, 136900, 136901) c6( ) c7(T, ea, fd, ci, bRn, aY, azY, aE, rw, dV, Ds, zy, hH, aa, yo, az, ih, vs, zC, GR, ba, fT, aZx, gt, cML, dc, Zn, bas, k, eg, nD, blB, aj, qW, cd, zo, lG, ey, pl, mR, Cw, sp, EU, ge, fQ, fe, CQ, adz, aBs, bbz, eD, pV, E, Dp, DM, aA, cz, bX, q, aWb, bNQ, Cc, aWc, yu, Di, cy, eu, le, baq, bg, aMA, iy, lo, pb, cT, ac, ig, bt, zA, bTc, ao, gV, BD, iW, jh, dG, fu, ad, hX, yt, oW, lQ, ckF, cH, cW, ff, dq, Sm, aFd, iV, bz, DF, xZ, dX, lC, ra, ne, iu, oO, bM, if, e, bB, cL, Tz, ip, x, bPx, ja, azm, bc, dH, fc, hl, kt, pD, eE, bw, cMN, lt, ki, mx, vy, tV, bjL, pJ, abh, ajy, dD, n, aCZ, ec, eV, hn, A, yq, dC, aOL, hL, at, ut, SZ, fl, eo, cB, B, ek, NO, dA, Lx, eX, fF, GC, v, V, rS, sl, Id, ae, cMK, hM, cMJ, iZ, DN, t, aff, asw, ck, cMx, cht, ro, JH, HQ, OR, wV, eq, Pm, afm, ds, XI, sj, CL, KG, Zf, NH, aFt, dN, aDN, D, gk, wz, MJ, gy, y, hv, pC, fR, atj, al, qj, lR, aMB, wa, bOl, AW, xE, u, Bc, M, K, aZS, DY, dF, eL, bzH, cMM, qS, asD, mQ, aAT, hN, aB, g, uC, aDs, bPC, uo, Hb, m, Le, sQ, aw, NT, BH, aBl, pM, pU, eh, Uh, kH, CU, afi, wb, cMO, aDp, nN, cM, fq, Zb, bMc, KT, ar, W, bbx, Br, gg, Pr, L, fM, AL, cMG, De, jx, aee, uZ, j, eA, J, o, hb, jZ, qG, bq, bNR, xR, Y, r, brW, ls, en, ik, Ym, and, hc, dh, tM, wR, cS, dy, EY, fD, sY, bjX, aFe, bQH, bS, dP, fU, io, ji, BN, cr, hp, iR, bPn, aFb, sG, cP, ei, xg, pf, eb, Jx, dY, YX, aaW, H, eN, hm, DA, LC, aW, arZ, eM, Jp, uJ, R, wh, My, ez, qN, ai, ql, ju, h, dL, ux, hB, he, OV, oo, bar, aCE, aq, Hu, dw, Wt, asE, vD, NL, hk, hR, uz, aDE, bL, Dw, lk, eR, aBo, hy, OY, C, ady, mP, uT, qp, afQ, aT, bK, an, qk, af, s, oV, aDm, ajl, wg, nM, pZ, aJt, vZ, WP, AH, fP, wq, CZ, aDj, Kd, Ke, aD, ha, aAk, vr, Fb, Jz, jE, mC, KL, LH, av, bPu, l, F, Os, jo, ul, yx, eZ, yy, aFr, aF, aDd, uF, ek, axs, atM, bE, z, aS, be, gq); #c1(TLR4) c2(NP_612564) c3 (toll-like receptor 4 isoform A precursor) c4(236683) c5(236684, 236685, 236686, 236687, 236688, 236689, 236690) c6( ) c7(T, ea, fd, ci, bRn, aY, azY, aE, rw, dV, Ds, zy, hH, aa, yo, az, ih, vs, zC, GR, ba, fT, aZx, gt, cML, dc, Zn, bas, k, eg, nD, blB, aj, qW, cd, zo, lG, ey, pl, mR, Cw, sp, EU, ge, fQ, fe, CQ, adz, aBs, bbz, eD, pV, E, Dp, DM, aA, cz, bX, q, aWb, bNQ, Cc, aWc, yu, Di, cy, eu, le, baq, bg, aMA, iy, lo, pb, cT, ac, ig, bt, zA, bTc, ao, gV, BD, iW, jh, dG, fu, ad, hX, yt, oW, lQ, ckF, cH, cW, ff, dq, Sm, aFd, iV, bz, DF, xZ, dX, lC, ra, ne, iu, oO, bM, if, e, bB, cL, Tz, ip, x, bPx, ja, azm, bc, dH, fc, hl, kt, pD, eE, bw, cMN, lt, ki, mx, vy, tV, bjL, pJ, abh, ajy, dD, n, aCZ, ec, eV, hn, A, yq, dC, aOL, hL, at, ut, SZ, fl, eo, cB, B, ek, NO, dA, Lx, eX, fF, GC, v, V, rS, sl, ld, ae, cMK, hM, cMJ, iZ, DN, t, aff, asw, ck, cMx, cht, ro, JH, HQ, OR, WV, eq, Pm, afm, ds, XI, sj, CL, hG, Zf, NH, aFt, dN, aDN, D, gk, wz, MJ, gy, y, hv, pC, fR, atj, al, qj, lR, aMB, wa, bOl, AW, xE, u, Bc, M, K, aZS, DY, dF, eL, bzH, cMM, qS, asD, md, aAT, hN, aB, g, uC, aDs, bPC, uo, Hb, m, Le, sl, aw, NT, BH, aBl, pM, pU, eh, Uh, KH, cU, aFi, wb, cMO, aDp, nN, cM, fq, Zb, bMc, KT, ar, W, bbx, Br, gg, Pr, L, fM, AL, cMG, oe, jx, aee, uZ, j, eA, J, o, hb, jZ, qG, bq, bNR, xR, Y, r, brW, ls, en, ik, Ym, and, hc, dh, tM, wR, cS, dy, EY, fD, sY, bjX, aFe, bQH, bS, dP, fU, io, ji, BN, cr, hp, iR, bPn, aFb, sG, cP, ei, xg, pf, eb, Jx, dY, YX, aaW, H, eN, hm, DA, LC, aW, arZ, eM, Jp, uJ, R, wh, My, ez, qN, ai, ql, ju, h, dl, ux, hB, he, OV, oo, bar, aCE, aq, Hu, dw, Wt, asE, vD, NL, hk, hR, uz, aDE, bL, Dw, lk, eR, aBo, hy, OY, C, ady, mP, uT, qp, afQ, aT, bK, an, qk, af, s, oV, aDm, ajl, wg, nM, pZ, aJt, vZ, WP, AH, fP, wq, CZ, aDj, Kd, Ke, aD, ha, aAk, vr, Fb, Jz, jE, mC, KL, LH, av, bPu, l, F, Os, jo, ul, yx, eZ, yy, afr, aF, aDd, uF, ek, axs, atM, bE, z, aS, be, gq); #c1(TLR5) c2(XP_006711569) c3(toll-like receptor 5 isoform X1) c4(286136, 286137) c5(286138, 286139, 286140, 286141, 286142, 286143, 286144, 286145, 286146, 286147, 286148, 286149, 286150, 286151) c6( ) c7(kV, bL, sY, DM, T, bM, FR, Dw, e, jh, aA, bB, aj, v, ci, C, uE, V, mC, J, cMP, q, ad, zT, wa, cF, aWc, ds, qp, Uh, aT, h, Ds, qG, ji, yb, DY, ao, aa, mR, aW, fO, az, eZ, r, fe, aF, uF, nN, kk, iV, uC, cMx, y, fT, eM, bE, wz, gZ, uz, Dt, fL, EY, fD, bNH, aSs, X, hv); #c1(TLR6) c2(NP_006059) c3(toll-like receptor 6 precursor) c4(161529, 161530, 161531, 161532) c5(161533, 161534, 161535, 161536, 161537, 161538, 161539, 161540, 161541, 161542, 161543, 161544, 161545, 161546, 161547, 161548, 161549, 161550, 161551, 161552, 161553, 161554, 161555, 161556, 161557, 161558, 161559, 161560) c6( ) c7(bM, sY, Dw, fR, Ke, iW, cz, fd, ci, C, aBl, Vh, atK, V, ad, wa, azY, cwM, aWc, jZ, sl, dV, aT, h, Ds, ae, sp, ml, Du, aFr, fe, uF, iV, eE, bw, uC, nM, ig, wz, at, wR, gy, uJ, EY, aDE, CZ, aNn, y, aDj); #c1(TLR7) c2(NP_057646) c3(toll-like receptor 7 precursor) c4(192027) c5(192028, 192029, 192030, 192031, 192032, 192033) c6( ) c7(Kd, T, dD, bM, BD, FR, sQ, Dw, ea, jx, eX, RC, jh, hy, v, eq, V, mC, q, ad, hX, wa, eW, b, F, aE, ds, afQ, M, Os, h, eV, Ds, ji, lQ, amA, ae, OV, eZ, ff, Y, yx, lb, fJ, cr, ks, dN, aq, lo, aDm, hp, CO, Fq, en, g, jT, fe, Wt, LB, sS, Bv, W, eM, il, hk, hc, cj, uo, ut, yH, fl, i, hE, B, y); #c1(TLR8) c2(NP_619542) c3(toll-like receptor 8 isoform 2 precursor) c4(236881) c5(236882, 236883, 236884, 236885, 236886) c6( ) c7(bM, T, os, Dw, BD, FR, sQ, jx, eX, RC, oZ, j, V, eW, ad, LH, wa, afQ, M, h, eV, Ds, ey, lQ, ae, blm, OV, aHW, Y, ff, Pg, cr, eZ, fe, aiT, lo, hp, CQ, Fq, en, pb, asE, ig, hk, Ei, uo, ut, fl, y, Ze); #c1(TLR8) c2(XP_011543832) c3(toll-like receptor 8 isoform X1) c4(342962) c5(342963, 342964, 342965, 342966, 342967, 342968, 342969) c6( ) c7(bM, T, os, Dw, BD, fR, sQ, jx, eX, RC, oZ, j, V, eW, ad, LH, wa, afQ, M, h, eV, Ds, ey, lQ, ae, blm, OV, aHW, Y, ff, Pg, cr, eZ, fe, aiT, lo, hp, CQ, Fq, en, pb, asE, ig, hk, Ei, uo, ut, fl, y, Ze); #c1(TLR9) c2(NP_059138) c3(toll-like receptor 9 precursor) c4(193230, 193231) c5(193232, 193233, 193234, 193235, 193236, 193237, 193238, 193239, 193240, 193241, 193242, 193243) c6( ) c7(oO, bM, T, aC, fR, if, e, fd, bB, ci, bRn, aY, wa, aE, dV, Ds, K, hH, bc, cp, bzH, bJX, aa, bkr, aW, az, ml, hN, g, eE, ve, uC, cm, bw, zV, lt, eM, mx, cj, uo, Zn, aCy, aGN, m, eg, fy, aSs, NK, ajy, dD, sQ, lo, ht, aw, n, aj, oY, fr, aBl, uE, qN, eh, ai, eW, pe, cU, h, hn, wb, ey, A, sp, EU, ge, fe, aCE, adz, Wt, hq, asE, kT, dB, aOL, hl, at, uz, ut, fl, Tr, it, B, bL, Dw, fM, dA, eR, jx, aA, aBo, v, j, C, V, q, J, eA, mP, bNQ, jZ, sl, apJ, aT, yu, Di, aFm, ae, af, uA, blm, le, xR, DN, r, afq, lo, en, cl, t, ck, cT, il, kP, ig, hc, wR, dy, yH, CMD, JH, fD, bt, vi, Kd, sY, aD, iW, jh, eq, jE, CMQ, mC, ad, hX, av, ds, oW, ji, lQ, uQ, CL, d, yx, ff, cr, eZ, aF, Sm, iV, bz, aYt, DF, LB, DT, bE, z, aDB, wz, aCl, y, iu); #c1(TLX1) c2(NP_001182446) c3(T-cell leukemia homeobox protein 1 isoform 2) c4( ) c5(74915, 74916, 74917, 74918, 74919) c6( ) c7(ck, ph, T, NA, fM, iM, x, je, jh, kH, adW, hn, k, il, hF); #c1(TLX1) c2(NP_005512) c3(T-cell leukemia homeobox protein 1 isoform 1) c4(156980, 156981) c5(156982, 156983, 156984, 156985, 156986, 156987, 156988, 156989) c6( ) c7(ck, ph, T, NA, fM, iM, x, je, jh, KH, adW, hn, k, il, hF); #c1(TLX2) c2(NP_057254) c3(T-cell leukemia homeobox protein 2) c4(189738) c5(189739, 189740, 189741, 189742, 189743, 189744, 189745) c6( ) c7(T, ha, cz, bS, ct, bh, cd, clk, bjV, cMR, JD, kH, K, fb, je, Z, t, iM, ac, uS, eo, m); #c1(TLX3) c2(NP_066305) c3(T-cell leukemia homeobox protein 3) c4(207161) c5(207162, 207163, 207164, 207165, 207166, 207167, 207168) c6( ) c7(ck, x, T, kH, hn, dL, k, ph, hF, el); #c1(TM4SF1) c2(NP_055035) c3(transmembrane 4 L6 family member 1) c4(178433, 178434) c5(178435, 178436, 178437, 178438, 178439, 178440, 178441, 178442, 178443, 178444, 178445, 178446) c6( ) c7(T, aE, aW, Y, aT, g, ae, ad); #c1(TM4SF20) c2(NP_079071) c3(transmembrane 4 LG family member 20) c4(216894, 216895) c5(216896, 216897, 216898, 216899, 216900, 216901, 216902, 216903, 216904, 216905, 216906) c6( ) c7(cMS, Kr, CD); #c1(TM4SF4) c2(NP_004608) c3 (transmembrane 4 L6 family member 4) c4(148143) c5(148144, 148145, 148146, 148147, 148148, 148149) c6( ) c7(T, kl, aZ, hl, aq, iR, B); #c1(TM4SF5) c2(NP_003954) c3(transmembrane 4 L6 family member 5 precursor) c4(142421) c5(142422, 142423, 142424, 142425, 142426, 142427) c6( ) c7(av, T, B); #c1(TM6SF2) c2(NP_001001524) c3(transmembrane 6 superfamily member 2) c4(18610, 18611, 18612) c5(18613, 18614, 18615, 18616, 18617, 18618, 18619, 18620, 18621, 18622, 18623, 18624, 18625, 18626, 18627, 18628, 18629, 18630) c6( ) c7(aw); #c1(TM7SF2) c2(NP_001264162) c3(delta(14)-sterol reductase isoform 2) c4(97608) c5(97609, 97610, 97611, 97612, 97613, 97614) c6( ) c7(ID, T, kh, dD, aC, ea, aw, al, jx, aA, cz, bB, aj, j, ci, eq, fr, cd, J, ai, eJ, Vs, Vp, av, iu, aE, lv, fm, Vr, jZ, pX, gd, aT, dV, cy, uD, cY, hm, ly, A, d, lR, nO, aW, xs, hl, aq, Vl, as, t, Vk, bz, lx, ek, gk, y, eM, dB, wO, bE, lI, aS, m, dy, bC, ei, lC, B, us); #c1(TM7SF2) c2(NP_003264) c3(delta(14)-sterol reductase isoform 1) c4(136924, 136925) c5(136926, 136927, 136928, 136929, 136930, 136931, 136932, 136933, 136934, 136935, 136936, 136937) c6( ) c7(ID, T, kh, dD, aC, ea, aw, al, jx, aA, cz, bB, aj, j, ci, eq, fr, cd, J, ai, eJ, Vs, Vp, av, iu, aE, lv, fm, Vr, jZ, pX, gd, aT, dV, cy, uD, cY, hm, ly, A, d, lR, nO, aW, xs, hl, aq, Vl, as, t, Vk, bz, lx, ek, gk, y, eM, dB, wl, bE, lI, aS, m, dy, bC, ei, lC, B, us); #c1(TM9SF2) c2(NP_004791) c3(transmembrane 9 superfamily member 2 precursor) c4(149885) c5(149886, 149887, 149888, 149889, 149890, 149891) c6( ) c7(aw, VZ, ey); #c1(TM9SF4) c2(NP_055557) c3(transmembrane 9 superfamily member 4 precursor) c4(181922) c5(181923, 181924, 181925, 181926, 181927, 181928, 181929, 181930) c6( ) c7(T, Y); #c1(TM9SF4) c2(XP_005260679) c3(transmembrane 9 superfamily member 4 isoform X1) c4(276805, 276806) c5(276807, 276808, 276809, 276810, 276811, 276812, 276813, 276814, 276815, 276816) c6( ) c7(T, Y); #c1(TMBIM4) c2(NP_001269535) c3(protein lifeguard 4 isoform a) c4(101830) c5(101831, 101832, 101833, 101834, 101835, 101836) c6( ) c7(bL, om, hl, cMT, lo, wR, aj, v, nS, ae, J, ad); #c1(TMBIM4) c2(NP_001269538) c3(protein lifeguard 4 isoform c) c4( ) c5(101837, 101838, 101839, 101840, 101841) c6( ) c7(bL, om, hl, cMT, lo, wR, aj, v, nS, ae, J, ad); #c1(TMBIM4) c2(NP_057140) c3(protein lifeguard 4 isoform b) c4(189081, 189082, 189083) c5(189084, 189085, 189086, 189087, 189088, 189089, 189090, 189091, 189092, 189093, 189094, 189095) c6( ) c7(bL, om, hl, cMT, lo, wR, aj, v, nS, ae, J, ad); #c1(TMBIM6) c2(NP_003208) c3(bax inhibitor 1 isoform 1) c4(136290, 136291, 136292, 136293, 136294, 136295, 136296, 136297, 136298, 136299) c5(136300, 136301, 136302, 136303, 136304, 136305, 136306, 136307, 136308, 136309, 136310, 136311, 136312, 136313, 136314, 136315, 136316, 136317, 136318, 136319, 136320, 136321, 136322, 136323, 136324, 136325, 136326, 136327, 136328, 136329, 136330, 136331, 136332, 136333, 136334, 136335, 136336, 136337, 136338, 136339, 136340, 136341, 136342, 136343, 136344, 136345, 136346, 136347, 136348, 136349, 136350, 136351, 136352, 136353, 136354, 136355, 136356, 136357, 136358, 136359, 136360, 136361, 136362, 136363, 136364, 136365, 136366, 136367, 136368, 136369, 136370, 136371, 136372) c6( ) c7(LB, ck, Mo, T, ac, iE, wD, aW, fG, NT, aT, lG, K, ci, g, bt, ae, af, ad); #c1(TMBIM6) c2(XP_005269183) c3(bax inhibitor 1 isoform X1) c4(282428, 282429, 282430) c5(282431, 282432, 282433, 282434, 282435, 282436, 282437, 282438, 282439, 282440, 282441, 282442, 282443, 282444, 282445, 282446, 282447, 282448, 282449, 282450, 282451, 282452, 282453, 282454, 282455, 282456, 282457, 282458, 282459, 282460, 282461, 282462, 282463, 282464, 282465, 282466, 282467, 282468, 282469, 282470, 282471, 282472, 282473, 282474, 282475) c6( ) c7(LB, ck, Mo, T, ac, iE, wO, aW, fG, NT, aT, lG, K, ci, g, bt, ae, af, ad); #c1(TMC1) c2(NP_619636) c3(transmembrane channel-like protein 1) c4(236913) c5(236914, 236915, 236916, 236917, 236918, 236919, 236920, 236921, 236922, 236923, 236924, 236925, 236926, 236927, 236928, 236929, 236930, 236931, 236932, 236933, 236934, 236935, 236936, 236937, 236938, 236939, 236940, 236941, 236942, 236943, 236944, 236945, 236946, 236947, 236948, 236949, 236950) c6( ) c7(cMV, cMU, mG, aF, Bh, bCJ); #c1(TMC1) c2(XP_011516515) c3(transmembrane channel-like protein 1 isoform X1) c4(309915, 309916, 309917, 309918) c5(309919) c6( ) c7(cMV, cMU, mG, aF, Bh, bCJ); #c1(TMC2) c2(NP_542789) c3(transmembrane channel-like protein 2) c4(233100) c5(233101, 233102, 233103, 233104, 233105, 233106) c6( ) c7(eZ); #c1(TMC3) c2(NP_001074001) c3(transmembrane channel-like protein 3) c4(36902, 36903) c5(36904, 36905, 36906, 36907, 36908, 36909, 36910, 36911, 36912, 36913, 36914, 36915) c6( ) c7(c); #c1(TMC5) c2(NP_001098718) c3 (transmembrane channel-like protein 5 isoform a) c4(41544) c5(41545, 41546, 41547, 41548, 41549, 41550, 41551, 41552, 41553, 41554) c6( ) c7(ff, ht, en); #c1(TMC5) c2(NP_001098719) c3(transmembrane channel-like protein 5 isoform b) c4(41555) c5(41556, 41557, 41558, 41559, 41560, 41561) c6( ) c7(ff, ht, en); #c1(TMC5) c2(NP_079056) c3(transmembrane channel-like protein 5 isoform c) c4(216788, 216789) c5(216790, 216791, 216792, 216793, 216794, 216795, 216796, 216797, 216798, 216799, 216800, 216801) c6( ) c7(ff, ht, en); #c1(TMC6) c2(NP_009198) c3(transmembrane channel-like protein 6) c4(171460) c5(171461, 171462, 171463, 171464, 171465, 171466, 171467, 171468, 171469, 171470, 171471, 171472, 171473, 171474) c6( ) c7(pa, lg, yk, if, ff, en, qN); #c1(TMC6) c2(XP_005257054) c3(transmembrane channel-like protein 6 isoform X3) c4(274646) c5(274647, 274648, 274649, 274650, 274651, 274652) c6( ) c7(pa, lg, yk, if, ff, en, qN); #c1(TMC6) c2(XP_011522558) c3(transmembrane channel-like protein 6 isoform X2) c4(317145) c5( ) c6( ) c7(pa, lg, yk, if, ff, en, qN); #c1(TMC6) c2(XP_011522559) c3(transmembrane channel-like protein 6 isoform X5) c4( ) c5(317146, 317147, 317148, 317149, 317150, 317151) c6( ) c7(pa, lg, yk, if, ff, en, qN); #c1(TMC6) c2(XP_011522560) c3(transmembrane channel-like protein 6 isoform X6) c4(317152) c5(317153, 317154, 317155, 317156, 317157, 317158) c6( ) c7(pa, lg, yk, if, ff, en, qN); #c1(TMC8) c2(NP_689681) c3(transmembrane channel-like protein 8) c4(243567) c5(243568, 243569, 243570, 243571, 243572, 243573) c6( ) c7(pa, lg, yk, if, ff, en, qN); #c1(TMC8) c2(XP_011522713) c3(transmembrane channel-like protein 8 isoform X9) c4(317235) c5(317236, 317237, 317238, 317239, 317240, 317241) c6( ) c7(pa, lg, yk, if, ff, en, qN); #c1(TMCC1) c2(XP_006713615) c3(transmembrane and coiled-coil domains protein 1 isoform X3) c4( ) c5(287241) c6( ) c7(if, qN, lg, apx); #c1(TMCC1) c2(XP_011510881) c3(transmembrane and coiled-coil domains protein 1 isoform X1) c4(301324, 301325) c5(301326, 301327, 301328, 301329, 301330, 301331, 301332, 301333) c6( ) c7(if, qN, lg, apx); #c1(TMCC1) c2(XP_011510883) c3(transmembrane and coiled-coil domains protein 1 isoform X2) c4(301334) c5(301335, 301336, 301337, 301338, 301339) c6( ) c7(if, qN, lg, apx); #c1(TMCC1) c2(XP_011510884) c3(transmembrane and coiled-coil domains protein 1 isoform X4) c4(301340) c5(301341, 301342, 301343, 301344, 301345, 301346) c6( ) c7(if, qN, lg, apx); #c1(TMCC1) c2(XP_011510886) c3(transmembrane and coiled-coil domains protein 1 isoform X6) c4( ) c5(301347, 301348, 301349, 301350, 301351) c6( ) c7(if, qN, lg, apx); #c1(TMCC2) c2(NP_001229854) c3(transmembrane and coiled-coil domains protein 2 isoform 2) c4(85428) c5(85429, 85430, 85431, 85432, 85433) c6( ) c7(afW); #c1(TMCC2) c2(NP_001284540) c3(transmembrane and coiled-coil domains protein 2 isoform 3) c4(112007) c5(112008, 112009, 112010, 112011, 112012, 112013, 112014, 112015, 112016, 112017, 112018, 112019) c6( ) c7(afW); #c1(TMCC2) c2(NP_001284542) c3(transmembrane and coiled-coil domains protein 2 isoform 4) c4(112020, 112021) c5(112022, 112023, 112024, 112025, 112026, 112027) c6( ) c7(afW); #c1(TMCC2) c2(NP_055673) c3(transmembrane and coiled-coil domains protein 2 isoform 1) c4(182671) c5(182672, 182673, 182674, 182675, 182676, 182677, 182678) c6( ) c7(afW); #c1(TMCC3) c2(NP_065749) c3(transmembrane and coiled-coil domains protein 3 isoform 1) c4(205176) c5(205177, 205178, 205179, 205180, 205181, 205182) c6( ) c7(cd, cz); #c1(TMC01) c2(NP_061899) c3(transmembrane and coiled-coil domain-containing protein 1 isoform a) c4(201049, 201050) c5(201051, 201052, 201053, 201054, 201055, 201056, 201057, 201058, 201059, 201060, 201061, 201062) c6( ) c7(aPn, pZ, dH, cMW, Az, cz, nA, dw, dr); #c1(TMC04) c2(XP_011539488) c3(transmembrane and coiled-coil domain-containing protein 4 isoform X1) c4(337678, 337679) c5(337680, 337681, 337682, 337683, 337684, 337685, 337686, 337687, 337688, 337689, 337690, 337691) c6( ) c7(bM); #c1(TMCO5A) c2(XP_005254225) c3(transmembrane and coiled-coil domain-containing protein 5A isoform X1) c4(273121, 273122) c5(273123, 273124, 273125, 273126, 273127, 273128) c6( ) c7(vr, fq, bK); #c1(TMCO5A) c2(XP_011519564) c3(transmembrane and coiled-coil domain-containing protein 5A isoform X2) c4(314197, 314198) c5( ) c6( ) c7(vr, fq, bK); #c1(TMCO5A) c2(XP_011519565) c3(transmembrane and coiled-coil domain-containing protein 5A isoform X3) c4(314199, 314200) c5(314201, 314202, 314203, 314204, 314205, 314206, 314207, 314208, 314209, 314210, 314211, 314212) c6( ) c7(vr, fq, bK); #c1(TMCO5A) c2(XP_011519566) c3(transmembrane and coiled-coil domain-containing protein 5A isoform X4) c4( ) c5(314213, 314214, 314215, 314216, 314217, 314218, 314219, 314220, 314221, 314222, 314223, 314224, 314225, 314226, 314227, 314228, 314229, 314230) c6( ) c7(vr, fq, bK); #c1(TMED1) c2(NP_006849) c3(transmembrane emp24 domain-containing protein 1 precursor) c4(168609) c5(168610, 168611, 168612, 168613, 168614, 168615) c6( ) c7(bM, dD, ea, e, h, wR, j, eq, eJ); #c1(TMED10) c2(NP_006818) c3(transmembrane emp24 domain-containing protein 10 precursor) c4(168372) c5(168373, 168374, 168375, 168376, 168377, 168378) c6( ) c7(T, eG, bP, Pg, U, aT, auT, alR, alQ, K, alP, ae, CW, ad); #c1(TMED2) c2(NP_006806) c3(transmembrane emp24 domain-containing protein 2 precursor) c4(168263, 168264, 168265) c5(168266, 168267, 168268, 168269, 168270, 168271, 168272, 168273, 168274, 168275, 168276, 168277, 168278, 168279, 168280, 168281, 168282, 168283) c6( ) c7(if, iW, j, qN, hX, ds, jZ, dV, kH, h, SH, ey, he, nJ, CB, je, aHW, lo, kk, ck, iM, il, cA, bE, NL, aEG, zv, aEN, EB); #c1(TMED3) c2(NP_001288132) c3(transmembrane emp24 domain-containing protein 3 isoform b precursor) c4( ) c5(113588, 113589, 113590, 113591, 113592, 113593) c6( ) c7(h, ae, ad); #c1(TMED3) c2(NP_031390) c3 (transmembrane emp24 domain-containing protein 3 isoform a precursor) c4(172187, 172188) c5(172189, 172190, 172191, 172192, 172193, 172194) c6( ) c7(h, ae, ad); #c1(TMED4) c2(NP_872353) c3(transmembrane emp24 domain-containing protein 4 isoform 1 precursor) c4(256176, 256177) c5(256178, 256179, 256180, 256181, 256182, 256183, 256184, 256185, 256186, 256187, 256188, 256189) c6( ) c7(g); #c1(TMED7) c2(NP_861974) c3(transmembrane emp24 domain-containing protein 7 precursor) c4(255742) c5(255743, 255744, 255745, 255746, 255747, 255748) c6( ) c7(T, ea, if, fd, bB, ci, aE, il, Qk, K, eL, bc, xQ, yo, aW, as, cm, bw, GR, eM, nB, dc, ki, go, vA, cj, m, Hb, dD, NT, n, fr, qN, jz, h, dL, ge, aq, zZ, iO, bP, hL, hR, it, B, fM, aoE, aA, q, eA, XX, aT, dz, ae, af, jf, s, il, Y, r, en, cl, t, ik, aEp, ck, nM, iM, dy, bt, On, hS, hl, Ke, pY, jh, eq, ad, hX, hd, ib, fb, d, je, et, ff, apb, aF, bz, bE, aS, EB); #c1(TMED7-TICAM2) c2(NP_001157940) c3(TRAM adaptor with GOLD domain isoform 1 precursor) c4(62897, 62898, 62899) c5(62900, 62901, 62902, 62903, 62904, 62905, 62906, 62907, 62908, 62909, 62910) c6( ) c7(T, ea, if, fd, bB, ci, aE, il, Qk, K, eL, bc, xQ, yo, aW, as, cm, bw, GR, eM, nB, dc, ki, go, vA, cj, m, Hb, dD, NT, n, fr, qN, jz, h, dl, ge, aq, zZ, iO, bP, hL, hR, it, B, fM, aDE, aA, q, eA, XX, aT, dz, ae, af, jf, s, il, Y, r, en, cl, t, ik, aEp, ck, nM, iM, dy, bt, On, hS, hl, Ke, pY, jh, eq, ad, hX, hd, ib, fb, d, je, et, ff, apb, aF, bz, bE, aS, EB); #c1(TMED7-TICAM2) c2(NP_001157941) c3(TRAM adaptor with GOLD domain isoform 2 precursor) c4( ) c5(62911, 62912, 62913, 62914, 62915, 62916, 62917, 62918, 62919, 62920, 62921) c6( ) c7(T, ea, if, fd, bB, ci, aE, il, Ok, K, eL, bc, xQ, yo, aW, as, cm, bw, GR, eM, nB, dc, ki, go, vA, cj, m, Hb, dD, NT, n, fr, qN, jz, h, dL, ge, aq, zZ, iO, bP, hL, hR, it, B, fM, aDE, aA, q, eA, XX, aT, dz, ae, af, jf, s, il, Y, r, en, cl, t, ik, aEp, ck, nM, iM, dy, bt, On, hS, hl, Ke, pY, jh, eq, ad, hX, hd, ib, fb, d, je, et, ff, apb, aF, bz, bE, aS, EB); #c1(TMED9) c2(NP_059980) c3(transmembrane emp24 domain-containing protein 9 precursor) c4(193463) c5(193464, 193465, 193466, 193467, 193468, 193469) c6( ) c7(Hx, ac, c, U, h, fr, gD, aR); #c1(TMEFF1) c2(NP_003683) c3(tomoregulin-1 precursor) c4(140178) c5(140179, 140180, 140181, 140182, 140183, 140184) c6( ) c7(dB, Na, T, Nc); #c1(TMEFF2) c2(NP_057276) c3(tomoregulin-2 precursor) c4(189808) c5(189809, 189810, 189811, 189812, 189813, 189814) c6( ) c7(T, Js, pf, zt, ht, aA, hz, aj, qj, q, ad, hd, no, aT, dL, YQ, ae, eL, amw, kl, B, s, aW, az, LZ, r, fe, aF, g, ahV, bF, y, nB, kz, hr, dX, np, zl, gT, it, X, Jo, pC); #c1(TMEM100) c2(NP_060756) c3(transmembrane protein 100) c4(197627) c5(197628, 197629, 197630, 197631, 197632, 197633, 197634, 197635, 197636, 197637, 197638, 197639, 197640, 197641) c6( ) c7(bz, ey); #c1(TMEM101) c2(XP_011523653) c3(transmembrane protein 101 isoform X2) c4(318144) c5(318145, 318146, 318147, 318148, 318149, 318150, 318151, 318152, 318153, 318154, 318155) c6( ) c7(v); #c1(TMEM101) c2(XP_011523655) c3(transmembrane protein 101 isoform X1) c4(318156) c5(318157, 318158, 318159, 318160, 318161) c6( ) c7(v); #c1(TMEM105) c2(NP_848615) c3(transmembrane protein 105) c4(253994) c5(253995, 253996, 253997, 253998, 253999, 254000) c6( ) c7(ac); #c1(TMEM106B) c2(XP_005249846) c3(transmembrane protein 106B isoform X1) c4(269895, 269896, 269897) c5(269898, 269899, 269900, 269901, 269902, 269903, 269904, 269905, 269906, 269907, 269908, 269909, 269910, 269911, 269912, 269913, 269914, 269915, 269916, 269917, 269918) c6( ) c7(VZ, bb, c, WY, Ol, aaE); #c1(TMEM108) c2(NP_001269794) c3(transmembrane protein 108 isoform 2 precursor) c4(102851) c5(102852, 102853, 102854, 102855, 102856, 102857) c6( ) c7(bL, v, an); #c1(TMEM108) c2(XP_011511399) c3(transmembrane protein 108 isoform X1) c4(301972, 301973, 301974, 301975, 301976) c5(301977, 301978, 301979, 301980, 301981, 301982, 301983, 301984, 301985, 301986, 301987, 301988, 301989, 301990, 301991, 301992, 301993, 301994, 301995, 301996, 301997, 301998, 301999, 302000, 302001, 302002, 302003, 302004, 302005, 302006, 302007, 302008, 302009, 302010) c6( ) c7(bL, v, an); #c1(TMEM11) c2(NP_003867) c3(transmembrane protein 11, mitochondrial) c4(141639) c5(141640, 141641, 141642, 141643, 141644, 141645) c6( ) c7(nv, bL, cfE, xH); #c1(TMEM114) c2(NP_001277026) c3(transmembrane protein 114 isoform c) c4(108984) c5(108985, 108986, 108987, 108988, 108989, 108990) c6( ) c7(arm); #c1(TMEM115) c2(NP_008955) c3(transmembrane protein 115) c4(169784) c5(169785, 169786, 169787, 169788, 169789, 169790) c6( ) c7(s); #c1(TMEM117) c2(NP_001273141) c3(transmembrane protein 117 isoform c) c4(104354) c5(104355, 104356, 104357, 104358, 104359, 104360) c6( ) c7(oe, fq); #c1(TMEM117) c2(NP_001273142) c3 (transmembrane protein 117 isoform d) c4( ) c5(104361, 104362, 104363, 104364, 104365, 104366) c6( ) c7(oe, fq); #c1(TMEM117) c2(XP_011537134) c3(transmembrane protein 117 isoform X1) c4(334862, 334863) c5(334864, 334865, 334866, 334867, 334868, 334869) c6( ) c7(oe, fq); #c1(TMEM126A) c2(NP_001231664) c3(transmembrane protein 126A isoform 2) c4( ) c5(87434, 87435, 87436, 87437, 87438) c6( ) c7(vd, awk, cMX); #c1(TMEM126A) c2(NP_115649) c3(transmembrane protein 126A isoform 1) c4(223447, 223448) c5(223449, 223450, 223451, 223452, 223453, 223454, 223455) c6( ) c7(vd, awk, cMX); #c1(TMEM127) c2(NP_001180233) c3(transmembrane protein 127) c4(73475) c5(73476, 73477, 73478, 73479, 73480, 73481, 73482, 73483, 73484, 73485) c6( ) c7(T, pX, Hu, fr, anx, dr); #c1(TMEM128) c2(NP_001284480) c3(transmembrane protein 128 isoform 1) c4(111856, 111857) c5(111858, 111859, 111860, 111861, 111862, 111863, 111864, 111865, 111866, 111867, 111868, 111869, 111870, 111871, 111872, 111873) c6( ) c7(an); #c1(TMEM128) c2(NP_116316) c3(transmembrane protein 128 isoform 2) c4(226127) c5(226128, 226129, 226130, 226131, 226132, 226133) c6( ) c7(an); #c1(TMEM132A) c2(NP_060340) c3(transmembrane protein 132A isoform a precursor) c4(195488) c5(195489, 195490, 195491, 195492, 195493, 195494, 195495, 195496) c6(195497, 195498) c7(T, j, K); #c1(TMEM132A) c2(NP_821174) c3(transmembrane protein 132A isoform b precursor) c4(253353) c5(253354, 253355, 253356, 253357, 253358, 253359, 253360) c6( ) c7(T, j, K); #c1(TMEM132B) c2(NP_443139) c3(transmembrane protein 1328 isoform a) c4(229619) c5(229620, 229621, 229622, 229623, 229624, 229625) c6( ) c7(Y, dY, bA, an); #c1(TMEM132C) c2(NP_001129575) c3(transmembrane protein 132C precursor) c4(51853) c5(51854, 51855, 51856, 51857, 51858, 51859) c6( ) c7(aj); #c1(TMEM132D) c2(NP_597705) c3(transmembrane protein 1320 precursor) c4(235079, 235080) c5(235081, 235082, 235083, 235084, 235085, 235086) c6( ) c7(bM, T, if, aw, ww, qN, hF, pd, x, ft, lg, kH, aT, h, abj, hn, afu, je, tb, aW, aq, cm, hq, ck, iM, cj, k, bt); #c1(TMEM132D) c2(XP_011536196) c3 (transmembrane protein 1320 isoform X1) c4( ) c5(333486, 333487, 333488, 333489, 333490, 333491) c6( ) c7(bM, T, if, aw, ww, qN, hF, pd, x, ft, lg, kH, aT, h, abj, hn, afu, je, tb, aW, aq, cm, hq, ck, iM, cj, k, bt); #c1(TMEM132E) c2(NP_001291367) c3(transmembrane protein 132E precursor) c4( ) c5(115756, 115757, 115758, 115759, 115760, 115761) c6( ) c7(aW, j, ac, aT); #c1(TMEM132E) c2(XP_011522615) c3(transmembrane protein 132E isoform X2) c4(317173) c5( ) c6( ) c7(aW, j, ac, aT); #c1(TMEM134) c2(NP_001072118) c3(transmembrane protein 134 isoform b) c4(35417) c5(35418, 35419, 35420, 35421, 35422, 35423) c6( ) c7(W); #c1(TMEM134) c2(NP_079400) c3(transmembrane protein 134 isoform a) c4(217924) c5(217925, 217926, 217927, 217928, 217929, 217930) c6( ) c7(W); #c1(TMEM135) c2(NP_001162195) c3(transmembrane protein 135 isoform 2) c4(66356, 66357) c5(66358, 66359, 66360, 66361, 66362, 66363, 66364, 66365, 66366, 66367, 66368, 66369, 66370, 66371, 66372, 66373, 66374, 66375) c6( ) c7(ac, aw, ey, nA, V, aj, bA); #c1(TMEM135) c2(NP_075069) c3(transmembrane protein 135 isoform 1) c4(213704, 213705, 213706) c5(213707, 213708, 213709, 213710, 213711, 213712, 213713, 213714, 213715, 213716, 213717, 213718) c6( ) c7(ac, aw, ey, nA, V, aj, bA); #c1(TMEM138) c2(NP_057548) c3(transmembrane protein 138) c4(191349) c5(191350, 191351, 191352, 191353, 191354, 191355) c6( ) c7(cMY); #c1(TMEM138) c2(XP_006718648) c3(transmembrane protein 138 isoform X1) c4(291949) c5(291950) c6( ) c7(cMY); #c1(TMEM138) c2(XP_011543402) c3 (transmembrane protein 138 isoform X5) c4( ) c5(342294, 342295, 342296, 342297, 342298) c6( ) c7(cMY); #c1(TMEM150B) c2(XP_011525158) c3(transmembrane protein 1508 isoform X1) c4(319597) c5(319598, 319599, 319600, 319601, 319602, 319603, 319604, 319605, 319606, 319607) c6( ) c7(gv); #c1(TMEM151A) c2(NP_694998) c3(transmembrane protein 151A) c4(245630) c5(245631, 245632, 245633, 245634, 245635, 245636) c6( ) c7(kl); #c1(TMEM151B) c2(NP_001131032) c3(transmembrane protein 151B) c4(52557, 52558) c5(52559, 52560, 52561, 52562, 52563, 52564, 52565, 52566, 52567, 52568, 52569, 52570) c6( ) c7(kl); #c1(TMEM154) c2(NP_689893) c3(transmembrane protein 154 precursor) c4(244276) c5(244277, 244278, 244279, 244280, 244281, 244282) c6( ) c7(fq); #c1(TMEM154) c2(XP_011530018) c3(transmembrane protein 154 isoform X1) c4(325815) c5(325816, 325817, 325818, 325819, 325820, 325821) c6( ) c7(fq); #c1(TMEM158) c2(NP_056259) c3(transmembrane protein 158 precursor) c4(186783) c5(186784, 186785, 186786, 186787, 186788, 186789, 186790) c6( ) c7(aW, T, fq, aT); #c1(TMEM160) c2(NP_060324) c3(transmembrane protein 160 precursor) c4(195396) c5(195397, 195398, 195399, 195400, 195401, 195402) c6( ) c7(v); #c1(TMEM161B) c2(NP_699185) c3(transmembrane protein 1618 isoform 2) c4(245813, 245814) c5(245815, 245816, 245817, 245818, 245819, 245820) c6( ) c7(bM); #c1(TMEM161B) c2(XP_006714616) c3(transmembrane protein 1618 isoform X2) c4( ) c5(287927, 287928, 287929, 287930, 287931, 287932) c6( ) c7(bM); #c1(TMEM163) c2(NP_112185) c3(transmembrane protein 163) c4(220667, 220668) c5(220669, 220670, 220671, 220672, 220673, 220674) c6( ) c7(an); #c1(TMEM163) c2(XP_011510251) c3(transmembrane protein 163 isoform X1) c4( ) c5(300857, 300858, 300859, 300860, 300861, 300862) c6( ) c7(an); #c1(TMEM165) c2(NP_060945) c3(transmembrane protein 165 precursor) c4(198840, 198841) c5(198842, 198843, 198844, 198845, 198846, 198847, 198848, 198849, 198850, 198851, 198852, 198853) c6( ) c7(arv, cMZ, Km, dv); #c1(TMEM169) c2(NP_001135784) c3(transmembrane protein 169) c4(53249, 53250, 53251) c5(53252, 53253, 53254, 53255, 53256, 53257, 53258, 53259, 53260, 53261, 53262, 53263, 53264, 53265, 53266, 53267, 53268, 53269, 53270, 53271, 53272, 53273, 53274, 53275, 53276, 53277, 53278, 53279, 53280, 53281) c6( ) c7(at, j); #c1(TMEM170A) c2(NP_660297) c3(transmembrane protein 170A isoform 1) c4(240781) c5(240782, 240783, 240784, 240785, 240786, 240787, 240788, 240789, 240790, 240791) c6( ) c7(j); #c1(TMEM171) c2(NP_001154814) c3(transmembrane protein 171 isoform 2) c4(61101) c5(61102, 61103, 61104, 61105, 61106, 61107) c6( ) c7(dS); #c1(TMEM171) c2(NP_775761) c3(transmembrane protein 171 isoform 1) c4(250203) c5(250204, 250205, 250206, 250207, 250208, 250209) c6( ) c7(dS); #c1(TMEM173) c2(NP_938023) c3(stimulator of interferon genes protein isoform 1) c4(259537, 259538) c5(259539, 259540, 259541, 259542, 259543, 259544) c6( ) c7(Uq, AH, jx, lo, bw, it); #c1(TMEM173) c2(XP_011535941) c3(stimulator of interferon genes protein isoform X2) c4( ) c5(333228, 333229, 333230, 333231, 333232, 333233) c6( ) c7(Uq, AH, jx, lo, bw, it); #c1(TMEM175) c2(NP_001284354) c3(transmembrane protein 175 isoform 2) c4(111758) c5( ) c6( ) c7(an); #c1(TMEM175) c2(NP_001284355) c3(transmembrane protein 175 isoform 3) c4( ) c5(111759, 111760, 111761, 111762, 111763) c6( ) c7(an); #c1(TMEM175) c2(NP_115702) c3(transmembrane protein 175 isoform 1) c4(223646) c5(223647, 223648, 223649, 223650, 223651, 223652, 223653) c6( ) c7(an); #c1(TMEM175) c2(XP_005272364) c3(transmembrane protein 175 isoform X5) c4(284004, 284005) c5(284006, 284007, 284008, 284009, 284010, 284011, 284012, 284013, 284014, 284015, 284016, 284017) c6( ) c7(an); #c1(TMEM176B) c2(NP_001094784) c3 (transmembrane protein 176B isoform b) c4(40705) c5(40706, 40707, 40708, 40709, 40710, 40711) c6( ) c7(H, fr, L); #c1(TMEM176B) c2(NP_054739) c3(transmembrane protein 1768 isoform a) c4(177500) c5(177501, 177502, 177503, 177504, 177505, 177506, 177507, 177508, 177509, 177510, 177511, 177512, 177513, 177514, 177515, 177516, 177517, 177518, 177519, 177520, 177521, 177522, 177523, 177524, 177525, 177526) c6( ) c7(H, fr, L); #c1(TMEM178A) c2(NP_689603) c3 (transmembrane protein 178A isoform 1 precursor) c4(243221, 243222) c5(243223, 243224, 243225, 243226, 243227, 243228, 243229) c6( ) c7(na); #c1(TMEM178A) c2(XP_005264202) c3(transmembrane protein 178A isoform X2) c4( ) c5(278700, 278701, 278702, 278703, 278704) c6( ) c7(na); #c1(TMEM18) c2(NP_690047) c3 (transmembrane protein 18) c4(244814, 244815) c5(244816, 244817, 244818, 244819, 244820, 244821, 244822, 244823, 244824, 244825, 244826, 244827) c6( ) c7(bL, bM, d, qQ, aj, v, fq, eA, J); #c1(TMEM182) c2(NP_653233) c3(transmembrane protein 182 precursor) c4(239258) c5(239259, 239260, 239261, 239262, 239263, 239264) c6( ) c7(dw, oe, bK, aw, EQ); #c1(TMEM182) c2(XP_011508933) c3(transmembrane protein 182 isoform X2) c4(299495) c5(299496, 299497, 299498, 299499, 299500, 299501) c6( ) c7(dw, oe, bk, aw, EQ); #c1(TMEM183A) c2(NP_612400) c3(transmembrane protein 183A) c4(236385) c5(236386, 236387, 236388, 236389, 236390, 236391) c6( ) c7(cz); #c1(TMEM184C) c2(NP_060711) c3(transmembrane protein 184C) c4(197335) c5(197336, 197337, 197338, 197339, 197340, 197341) c6( ) c7(hd, nB); #c1(TMEM185A) c2(NP_001167563) c3(transmembrane protein 185A isoform 2) c4(69266) c5(69267, 69268, 69269, 69270, 69271, 69272, 69273, 69274, 69275, 69276, 69277, 69278) c6( ) c7(nf, nA); #c1(TMEM185A) c2(NP_115897) c3(transmembrane protein 185A isoform 1) c4(224563, 224564) c5(224565, 224566, 224567, 224568, 224569, 224570) c6( ) c7(nf, nA); #c1(TMEM187) c2(XP_011529501) c3(transmembrane protein 187 isoform X1) c4(324980, 324981, 324982) c5(324983, 324984, 324985, 324986, 324987, 324988, 324989, 324990, 324991, 324992, 324993, 324994, 324995, 324996, 324997, 324998, 324999, 325000) c6( ) c7(eZ); #c1(TMEM189) c2(NP_001155977) c3 (transmembrane protein 189 isoform 2) c4(61680) c5(61681, 61682, 61683, 61684, 61685, 61686, 61687, 61688) c6( ) c7(T, dA, WT, ci, PJ, eW, ad, hX, h, BZ, K, ae, avl, ak, anR, d, CT, lo, as, pb, ck, Qi, iO, vW, c, iT, eB, U, bC, bt, xg, B); #c1(TMEM189) c2(NP_954580) c3(transmembrane protein 189 isoform 1) c4(261044, 261045) c5(261046, 261047, 261048, 261049, 261050, 261051, 261052, 261053, 261054, 261055, 261056, 261057) c6( ) c7(T, dA, WT, ci, PJ, eW, ad, hX, h, BZ, K, ae, avQ, ak, anR, d, CT, lo, as, pb, ck, Di, iO, vW, c, iT, eB, U, bC, bt, xg, B); #c1(TMEM189-UBE2V1) c2(NP_954673) c3(TMEM189-UBE2V1 fusion protein) c4(261240) c5(261241, 261242, 261243, 261244, 261245, 261246, 261247, 261248, 261249, 261250) c6( ) c7(T, dA, WT, ci, PJ, eW, ad, hX, cF, h, BZ, K, ae, avQ, ak, anR, d, CT, lo, as, pb, ck, Qi, iO, vW, c, iT, eB, U, bC, bt, xg, B); #c1(TMEM199) c2(NP_689677) c3(transmembrane protein 199) c4(243540, 243541) c5(243542, 243543, 243544, 243545, 243546, 243547, 243548, 243549, 243550, 243551, 243552) c6( ) c7(aW, aT); #c1(TMEM2) c2(NP_001129292) c3(transmembrane protein 2 isoform b) c4(51363) c5(51364, 51365, 51366, 51367, 51368, 51369, 51370, 51371, 51372, 51373, 51374, 51375, 51376) c6( ) c7(ae, oe, ad); #c1(TMEM2) c2(NP_037522) c3(transmembrane protein 2 isoform a) c4(175991, 175992, 175993) c5(175994, 175995, 175996, 175997, 175998, 175999, 176000, 176001, 176002, 176003, 176004) c6( ) c7(ae, oe, ad); #c1(TMEM200A) c2(NP_001245206) c3(transmembrane protein 200A) c4(92695) c5(92696, 92697, 92698, 92699, 92700, 92701, 92702, 92703, 92704, 92705, 92706, 92707, 92708, 92709) c6( ) c7(jf, fq); #c1(TMEM205) c2(NP_001138888) c3(transmembrane protein 205) c4(58086) c5(58087, 58088, 58089, 58090, 58091, 58092, 58093, 58094, 58095, 58096, 58097, 58098, 58099, 58100) c6( ) c7(T); #c1(TMEM207) c2(NP_997199) c3(transmembrane protein 207 precursor) c4(265166) c5(265167, 265168, 265169, 265170, 265171, 265172) c6( ) c7(YL, T, j); #c1(TMEM209) c2(NP_001288092) c3(transmembrane protein 209 isoform 2) c4(113505, 113506) c5(113507, 113508, 113509, 113510, 113511, 113512, 113513, 113514, 113515, 113516, 113517, 113518, 113519, 113520, 113521, 113522, 113523, 113524, 113525, 113526, 113527, 113528, 113529, 113530) c6( ) c7(bz); #c1(TMEM209) c2(NP_116231) c3(transmembrane protein 209 isoform 1) c4(225865, 225866) c5(225867, 225868, 225869, 225870, 225871, 225872, 225873, 225874, 225875, 225876, 225877) c6( ) c7(bz); #c1(TMEM209) c2(XP_011514950) c3(transmembrane protein 209 isoform X2) c4(307713) c5( ) c6( ) c7(bz); #c1(TMEM209) c2(XP_011514951) c3 (transmembrane protein 209 isoform X3) c4(307714) c5(307715) c6( ) c7(bz); #c1(TMEM213) c2(NP_001078898) c3(transmembrane protein 213 precursor) c4(37998) c5(37999, 38000, 38001, 38002, 38003) c6( ) c7(aC, py); #c1(TMEM213) c2(XP_005250224) c3(transmembrane protein 213 isoform X1) c4(270213) c5(270214, 270215, 270216, 270217, 270218, 270219, 270220) c6( ) c7(aC, py); #c1(TMEM215) c2(NP_997723) c3(transmembrane protein 215) c4(265783) c5(265784, 265785, 265786, 265787, 265788, 265789) c6( ) c7(oe); #c1(TMEM216) c2(NP_001167461) c3(transmembrane protein 216 isoform 2) c4(68980) c5(68981, 68982, 68983, 68984, 68985, 68986) c6( ) c7(ago, cNb, cNa, vm, zG, dr); #c1(TMEM216) c2(NP_001167462) c3(transmembrane protein 216 isoform 3) c4(68987) c5(68988, 68989, 68990, 68991, 68992, 68993) c6( ) c7(ago, cNb, cNa, vm, zG, dr); #c1(TMEM217) c2(NP_001273330) c3(transmembrane protein 217 isoform 3) c4(104690) c5(104691, 104692, 104693, 104694, 104695, 104696) c6( ) c7(af); #c1(TMEM217) c2(NP_660359) c3(transmembrane protein 217 isoform 1) c4(241096) c5(241097, 241098, 241099, 241100, 241101, 241102) c6( ) c7(af); #c1(TMEM217) c2(XP_011512667) c3(transmembrane protein 217 isoform X2) c4( ) c5(303687) c6( ) c7(af); #c1(TMEM219) c2(NP_001077082) c3(insulin-like growth factor-binding protein 3 receptor precursor) c4(37686) c5(37687, 37688, 37689, 37690, 37691, 37692, 37693, 37694, 37695, 37696) c6( ) c7(ae, T, bM, aW, aT, bz, ad); #c1(TMEM220) c2(NP_001004313) c3(transmembrane protein 220 precursor) c4(20546) c5(20547, 20548, 20549, 20550, 20551, 20552) c6( ) c7(aw); #c1(TMEM220) c2(XP_005256691) c3(transmembrane protein 220 isoform X1) c4(274296) c5(274297, 274298, 274299, 274300, 274301, 274302) c6( ) c7(aw); #c1(TMEM229A) c2(NP_001129474) c3(transmembrane protein 229A) c4(51554) c5(51555, 51556, 51557, 51558, 51559, 51560) c6( ) c7(fq); #c1(TMEM231) c2(NP_001070884) c3(transmembrane protein 231 isoform 1) c4(34890) c5(34891, 34892, 34893, 34894, 34895, 34896, 34897) c6( ) c7(cNc, ago, cNd); #c1(TMEM231) c2(NP_001070886) c3(transmembrane protein 231 isoform 2) c4(34898) c5(34899, 34900, 34901, 34902, 34903, 34904) c6( ) c7(cNc, ago, cNd); #c1(TMEM233) c2(NP_001130006) c3(transmembrane protein 233) c4(52406) c5(52407, 52408, 52409, 52410, 52411, 52412) c6( ) c7(hH, j); #c1(TMEM237) c2(NP_001037850) c3(transmembrane protein 237 isoform a) c4(34143, 34144) c5(34145, 34146, 34147, 34148, 34149, 34150, 34151, 34152, 34153, 34154, 34155, 34156) c6( ) c7(cNe); #c1(TMEM237) c2(NP_689601) c3(transmembrane protein 237 isoform b) c4(243189, 243190, 243191) c5(243192, 243193, 243194, 243195, 243196, 243197, 243198, 243199, 243200, 243201, 243202, 243203, 243204, 243205, 243206, 243207, 243208, 243209) c6( ) c7(cNe); #c1(TMEM241) c2(NP_116322) c3(transmembrane protein 241) c4(226145) c5(226146, 226147, 226148, 226149, 226150, 226151, 226152, 226153, 226154, 226155, 226156, 226157) c6( ) c7(ac); #c1(TMEM241) c2(XP_005258430) c3(transmembrane protein 241 isoform X4) c4( ) c5(275669, 275670, 275671, 275672, 275673) c6( ) c7(ac); #c1(TMEM241) c2(XP_006722624) c3(transmembrane protein 241 isoform X6) c4(294779) c5(294780, 294781, 294782, 294783, 294784, 294785) c6( ) c7(ac); #c1(TMEM241) c2(XP_011524534) c3(transmembrane protein 241 isoform X1) c4(319110) c5(319111) c6( ) c7(ac); #c1(TMEM241) c2(XP_011524535) c3(transmembrane protein 241 isoform X2) c4(319112) c5( ) c6( ) c7(ac); #c1(TMEM244) c2(NP_001010876) c3(transmembrane protein 244) c4(23898) c5(23899, 23900, 23901, 23902, 23903, 23904, 23905, 23906, 23907, 23908) c6( ) c7(v, cd); #c1(TMEM245) c2(NP_114401) c3 (transmembrane protein 245) c4(222531, 222532) c5(222533, 222534, 222535, 222536, 222537, 222538, 222539, 222540, 222541, 222542, 222543, 222544) c6( ) c7(ad); #c1(TMEM245) c2(XP_011516749) c3(transmembrane protein 245 isoform X2) c4(310206) c5(310207, 310208, 310209, 310210, 310211, 310212) c6( ) c7(ad); #c1(TMEM25) c2(NP_001137507) c3(transmembrane protein 25 isoform 2 precursor) c4(55941, 55942) c5(55943, 55944, 55945, 55946, 55947, 55948, 55949, 55950, 55951, 55952, 55953, 55954, 55955, 55956, 55957, 55958) c6( ) c7(aW, aT); #c1(TMEM25) c2(NP_001137508) c3(transmembrane protein 25 isoform 3 precursor) c4(55959) c5(55960, 55961, 55962, 55963, 55964, 55965) c6( ) c7(aW, aT); #c1(TMEM25) c2(NP_001137509) c3(transmembrane protein 25 isoform 4 precursor) c4(55966) c5(55967, 55968, 55969, 55970, 55971, 55972) c6( ) c7(aW, aT); #c1(TMEM25) c2(NP_001137510) c3(transmembrane protein 25 isoform 5 precursor) c4(55973) c5(55974, 55975, 55976, 55977, 55978, 55979, 55980, 55981, 55982, 55983, 55984, 55985) c6( ) c7(aW, aT); #c1(TMEM25) c2(NP_116169) c3(transmembrane protein 25 isoform 1 precursor) c4(225536) c5(225537, 225538, 225539, 225540, 225541, 225542) c6( ) c7(aW, aT); #c1(TMEM25) c2(XP_011541338) c3(transmembrane protein 25 isoform X2) c4(339943) c5(339944, 339945, 339946, 339947, 339948, 339949) c6( ) c7(aW, aT); #c1(TMEM259) c2(NP_001028198) c3(membralin isoform 1) c4(30360) c5(30361, 30362, 30363, 30364, 30365, 30366) c6( ) c7(ci); #c1(TMEM259) c2(NP_219488) c3(membralin isoform 2) c4( ) c5(228588, 228589, 228590, 228591, 228592, 228593, 228594) c6( ) c7(ci); #c1(TMEM259) c2(XP_005259733) c3(membralin isoform X2) c4(276279) c5( ) c6( ) c7(ci); #c1(TMEM261) c2(NP_219500) c3(transmembrane protein 261) c4(228653) c5(228654, 228655, 228656, 228657, 228658, 228659) c6( ) c7(alv, ad); #c1(TMEM27) c2(NP_065716) c3(collectrin precursor) c4(205056) c5(205057, 205058, 205059, 205060, 205061, 205062) c6( ) c7(amA, dy, wS, KW); #c1(TMEM30A) c2(NP_001137430) c3(cell cycle control protein 50A isoform 2) c4(55679, 55680) c5(55681, 55682, 55683, 55684, 55685, 55686, 55687, 55688, 55689, 55690, 55691, 55692, 55693, 55694, 55695, 55696) c6( ) c7(By); #c1(TMEM30A) c2(NP_060717) c3(cell cycle control protein 50A isoform 1) c4(197431, 197432, 197433) c5(197434, 197435, 197436, 197437, 197438, 197439, 197440, 197441, 197442, 197443, 197444, 197445, 197446, 197447, 197448, 197449, 197450, 197451, 197452, 197453) c6( ) c7(ey); #c1(TMEM30B) c2(NP_001017970) c3(cell cycle control protein 508) c4(26691, 26692) c5(26693, 26694, 26695, 26696, 26697, 26698, 26699, 26700, 26701, 26702) c6( ) c7(Y, bjl, ip); #c1(TMEM37) c2(NP_899063) c3(voltage-dependent calcium channel gamma-like subunit) c4(257938) c5(257939, 257940, 257941, 257942, 257943, 257944) c6( ) c7(zQ, ci); #c1(TMEM38A) c2(NP_076979) c3(trimeric intracellular cation channel type A) c4(214564) c5(214565, 214566, 214567, 214568, 214569, 214570) c6( ) c7(qa, cz); #c1(TMEM38B) c2(NP_060582) c3 (trimeric intracellular cation channel type B) c4(196673, 196674, 196675) c5(196676, 196677, 196678, 196679, 196680, 196681, 196682) c6( ) c7(cNf, v, zp); #c1(TMEM38B) c2(XP_005252132) c3(trimeric intracellular cation channel type B isoform X1) c4( ) c5(271567, 271568, 271569, 271570, 271571, 271572) c6( ) c7(cNf, v, zp); #c1(TMEM38B) c2(XP_005252133) c3(trimeric intracellular cation channel type B isoform X2) c4(271573) c5(271574, 271575, 271576, 271577, 271578, 271579) c6( ) c7(cNf, v, zp); #c1(TMEM38B) c2(XP_005252135) c3(trimeric intracellular cation channel type B isoform X6) c4( ) c5(271580, 271581, 271582, 271583, 271584, 271585) c6( ) c7(cNf, v, zp); #c1(TMEM38B) c2(XP_011517135) c3(trimeric intracellular cation channel type B isoform X7) c4(310663) c5(310664, 310665, 310666, 310667, 310668) c6( ) c7(cNf, v, zp); #c1(TMEM39A) c2(NP_060736) c3(transmembrane protein 39A) c4(197547, 197548) c5(197549, 197550, 197551, 197552, 197553, 197554) c6( ) c7(V, ac, eZ); #c1(TMEM39A) c2(XP_005247635) c3(transmembrane protein 39A isoform X2) c4(267891, 267892) c5( ) c6( ) c7(V, ac, eZ); #c1(TMEM39A) c2(XP_011511260) c3(transmembrane protein 39A isoform X3) c4( ) c5(301750, 301751, 301752, 301753, 301754, 301755, 301756, 301757, 301758, 301759, 301760, 301761, 301762, 301763, 301764, 301765, 301766, 301767) c6( ) c7(V, ac, eZ); #c1(TMEM40) c2(NP_001271335) c3(transmembrane protein 40 isoform a) c4(103904, 103905) c5(103906, 103907, 103908, 103909, 103910, 103911, 103912, 103913) c6( ) c7(WY); #c1(TMEM40) c2(NP_001271336) c3 (transmembrane protein 40 isoform c) c4(103914) c5(103915, 103916, 103917, 103918, 103919, 103920) c6( ) c7(WY); #c1(TMEM40) c2(NP_001271337) c3(transmembrane protein 40 isoform d) c4(103921) c5(103922, 103923, 103924, 103925, 103926, 103927) c6( ) c7(WY); #c1(TMEM40) c2(NP_060776) c3(transmembrane protein 40 isoform b) c4( ) c5(197726, 197727, 197728, 197729, 197730, 197731, 197732, 197733) c6( ) c7(WY); #c1(TMEM43) c2(NP_077310) c3(transmembrane protein 43) c4(215186) c5(215187, 215188, 215189, 215190, 215191, 215192) c6( ) c7(cc, wK, cAq, oj, Fw, cd, cNg); #c1(TMEM45A) c2(NP_060474) c3(transmembrane protein 45A) c4( ) c5(196124, 196125, 196126, 196127, 196128) c6( ) c7(T, aW, aT, aq, iR, B); #c1(TMEM45A) c2(XP_005247626) c3(transmembrane protein 45A isoform X1) c4(267882, 267883) c5(267884, 267885, 267886, 267887, 267888, 267889, 267890) c6( ) c7(T, aW, aT, aq, iR, B); #c1(TMEM47) c2(NP_113630) c3(transmembrane protein 47) c4(221817) c5(221818, 221819, 221820, 221821, 221822, 221823) c6( ) c7(nf); #c1(TMEM5) c2(NP_055069) c3(transmembrane protein 5 isoform 1) c4(178740) c5(178741, 178742, 178743, 178744, 178745, 178746) c6( ) c7(vJ, UD, cNh, ci, Cd); #c1(TMEM50B) c2(NP_006125) c3(transmembrane protein 508) c4(162225, 162226, 162227, 162228) c5(162229, 162230, 162231, 162232, 162233, 162234, 162235, 162236, 162237, 162238, 162239, 162240, 162241, 162242, 162243, 162244, 162245, 162246, 162247, 162248, 162249, 162250, 162251, 162252, 162253, 162254, 162255, 162256, 162257, 162258, 162259) c6( ) c7(T); #c1(TMEM55A) c2(NP_061180) c3(type 2 phosphatidylinositol 4,5-bisphosphate 4-phosphatase) c4(199797) c5(199798, 199799, 199800, 199801, 199802, 199803) c6( ) c7(an); #c1(TMEM57) c2(XP_011540006) c3(macoilin isoform X2) c4(338320) c5(338321, 338322, 338323, 338324, 338325, 338326) c6( ) c7(j); #c1(TMEM60) c2(NP_116325) c3(transmembrane protein 60) c4(226165) c5(226166, 226167, 226168, 226169, 226170, 226171) c6( ) c7(eR); #c1(TMEM62) c2(NP_079232) c3(transmembrane protein 62) c4(217555) c5(217556, 217557, 217558, 217559, 217560, 217561) c6( ) c7(bM); #c1(TMEM63A) c2(NP_055513) c3(CSC1-like protein 1) c4(181628, 181629, 181630) c5(181631, 181632, 181633, 181634, 181635, 181636) c6( ) c7(aA); #c1(TMEM63A) c2(XP_006711905) c3(CSC1-like protein 1 isoform X5) c4( ) c5(286294, 286295, 286296, 286297, 286298, 286299, 286300, 286301, 286302, 286303, 286304, 286305) c6( ) c7(aA); #c1(TMEM67) c2(NP_001135773) c3(Meckelin isoform 2) c4(53213) c5(53214, 53215, 53216, 53217, 53218, 53219) c6( ) c7(aBW, ul, ago, arS, dO, al, cNi, hl, wB, KA, KF, vm, zG, cqq, Tp, cNj, cd); #c1(TMEM67) c2(NP_714915) c3(Meckelin isoform 1 precursor) c4(246618) c5(246619, 246620, 246621, 246622, 246623, 246624) c6( ) c7(aBW, ul, ago, arS, dO, al, cNi, hl, wB, KA, KF, vm, zG, cqq, Tp, cNj, cd); #c1(TMEM70) c2(NP_001035703) c3(transmembrane protein 70, mitochondrial isoform b) c4( ) c5(32970, 32971, 32972, 32973, 32974) c6( ) c7(ID, ly, cc, aW, cmH, cmG, aT, oj, aly, aNG, Pp, cNk, px, kC, lx, akP); #c1(TMEM70) c2(NP_060336) c3(transmembrane protein 70, mitochondrial isoform a) c4(195449, 195450) c5(195451, 195452, 195453, 195454, 195455, 195456, 195457) c6( ) c7(ID, ly, cc, aW, cmH, cmG, aT, oj, aly, aNG, Pp, cNk, px, kC, lx, akP); #c1(TMEM74B) c2(NP_001291677) c3(transmembrane protein 74B isoform 1) c4(116328, 116329) c5(116330, 116331, 116332, 116333, 116334, 116335, 116336, 116337, 116338, 116339, 116340, 116341) c6( ) c7(V); #c1(TMEM79) c2(NP_115699) c3(transmembrane protein 79) c4(223617, 223618) c5(223619, 223620, 223621, 223622, 223623, 223624, 223625, 223626, 223627, 223628, 223629, 223630, 223631) c6( ) c7(eE); #c1(TMEM87A) c2(NP_001103973) c3(transmembrane protein 87A isoform 2 precursor) c4(42049) c5(42050, 42051, 42052, 42053, 42054, 42055) c6( ) c7(ac); #c1(TMEM87A) c2(NP_001273416) c3(transmembrane protein 87A isoform 3) c4(104852) c5(104853, 104854, 104855, 104856, 104857, 104858) c6( ) c7(ac); #c1(TMEM87A) c2(NP_056312) c3(transmembrane protein 87A isoform 1 precursor) c4(187028, 187029) c5(187030, 187031, 187032, 187033, 187034, 187035, 187036, 187037, 187038, 187039, 187040, 187041) c6( ) c7(ac); #c1(TMEM88) c2(NP_981956) c3(transmembrane protein 88) c4(263198) c5(263199, 263200, 263201, 263202, 263203, 263204) c6( ) c7(bz); #c1(TMEM89) c2(NP_001008270) c3(transmembrane protein 89 precursor) c4(23124) c5(23125, 23126, 23127, 23128, 23129, 23130) c6( ) c7(oe); #c1(TMEM8B) c2(NP_001036055) c3(transmembrane protein 88 isoform a) c4(33890) c5(33891, 33892, 33893, 33894, 33895, 33896, 33897, 33898) c6( ) c7(T, bE, aW, aA, r, aT, bB, aF, q); #c1(TMEM8B) c2(NP_057530) c3(transmembrane protein 8B isoform b) c4( ) c5(191267, 191268, 191269, 191270, 191271, 191272, 191273, 191274) c6( ) c7(T, bE, aW, aA, r, aT, bB, aF, q); #c1(TMEM8B) c2(XP_011516206) c3(transmembrane protein 8B isoform X5) c4(309436) c5(309437, 309438, 309439, 309440) c6( ) c7(T, bE, aW, aA, r, aT, bB, aF, q); #c1(TMEM95) c2(XP_005256677) c3(transmembrane protein 95 isoform X2) c4(274288) c5(274289, 274290, 274291, 274292, 274293, 274294, 274295) c6( ) c7(aer); #c1(TMEM95) c2(XP_011522119) c3(transmembrane protein 95 isoform X1) c4(316752) c5(316753, 316754, 316755, 316756, 316757, 316758) c6( ) c7(aer); #c1(TMEM97) c2(NP_055388) c3 (transmembrane protein 97) c4(180719) c5(180720, 180721, 180722, 180723, 180724, 180725, 180726, 180727, 180728, 180729, 180730, 180731) c6( ) c7(t, q, m, eM, r); #c1(TMEM97) c2(XP_005258022) c3(transmembrane protein 97 isoform X1) c4(275456) c5( ) c6( ) c7(t, q, m, eM, r); #c1(TMEM98) c2(NP_001288675) c3(transmembrane protein 98) c4(113920, 113921, 113922, 113923) c5(113924, 113925, 113926, 113927, 113928, 113929, 113930, 113931, 113932, 113933, 113934, 113935, 113936, 113937, 113938, 113939, 113940, 113941, 113942, 113943, 113944, 113945, 113946, 113947, 113948, 113949, 113950) c6( ) c7(B, bK, bX); #c1(TMF1) c2(NP_009045) c3(TATA element modulatory factor) c4(170392, 170393) c5(170394, 170395, 170396, 170397, 170398, 170399, 170400, 170401, 170402, 170403, 170404, 170405, 170406) c6( ) c7(kl, K, ad); #c1(TMF1) c2(XP_011532358) c3(TATA element modulatory factor isoform X1) c4(328518, 328519) c5(328520, 328521, 328522, 328523, 328524, 328525, 328526, 328527, 328528, 328529, 328530, 328531) c6( ) c7(kl, K, ad); #c1(TMIE) c2(NP_671729) c3(transmembrane inner ear expressed protein precursor) c4(242174) c5(242175, 242176, 242177, 242178, 242179, 242180) c6( ) c7(cNl, Bh, beM, bCy, bCd); #c1(TMIE) c2(XP_011531876) c3(transmembrane inner ear expressed protein isoform X1) c4( ) c5(327929) c6( ) c7(cNl, Bh, beM, bCy, bCd); #c1(TMIGD2) c2(NP_653216) c3 (transmembrane and immunoglobulin domain-containing protein 2 isoform 1 precursor) c4(239136) c5(239137, 239138, 239139, 239140, 239141, 239142) c6( ) c7(K); #c1(TMIGD3) c2(NP_001289609) c3(transmembrane and immunoglobulin domain containing 3 isoform 4) c4(114296, 114297) c5(114298, 114299, 114300, 114301, 114302, 114303, 114304, 114305, 114306, 114307, 114308, 114309, 114310, 114311, 114312, 114313, 114314, 114315) c6( ) c7(bL, fe, fD, iV); #c1(TMLHE) c2(NP_001171726) c3 (trimethyllysine dioxygenase, mitochondrial isoform 2 precursor) c4(71463) c5(71464, 71465, 71466, 71467, 71468, 71469) c6( ) c7(bN, cNm, nA, rz); #c1(TMLHE) c2(NP_060666) c3(trimethyllysine dioxygenase, mitochondrial isoform 1 precursor) c4(197066) c5(197067, 197068, 197069, 197070, 197071, 197072) c6( ) c7(bN, cNm, nA, rz); #c1(TMOD1) c2(NP_001159588) c3(tropomodulin-1) c4(64392, 64393) c5(64394, 64395, 64396, 64397, 64398, 64399, 64400, 64401, 64402, 64403, 64404, 64405, 64406, 64407, 64408, 64409) c6( ) c7(id, ad); #c1(TMOD2) c2(NP_001136357) c3(tropomodulin-2 isoform b) c4(54852) c5(54853, 54854, 54855, 54856, 54857, 54858) c6( ) c7(oe, id); #c1(TMOD2) c2(NP_055363) c3(tropomodulin-2 isoform a) c4(180605) c5(180606, 180607, 180608, 180609, 180610, 180611) c6( ) c7(oe, id); #c1(TMOD3) c2(NP_055362) c3(tropomodulin-3) c4(180587, 180588) c5(180589, 180590, 180591, 180592, 180593, 180594, 180595, 180596, 180597, 180598, 180599, 180600, 180601, 180602, 180603, 180604) c6( ) c7(oe, id); #c1(TMOD4) c2(NP_037485) c3(tropomodulin-4) c4(175794, 175795) c5(175796, 175797, 175798, 175799, 175800, 175801, 175802, 175803, 175804, 175805, 175806, 175807) c6( ) c7(T); #c1(TMOD4) c2(XP_005245161) c3(tropomodulin-4 isoform X2) c4(266332) c5(266333, 266334, 266335, 266336, 266337, 266338) c6( ) c7(T); #c1(TMPO) c2(NP_001027454) c3(thymopoietin isoform beta) c4(30188) c5(30189, 30190, 30191, 30192, 30193, 30194) c6( ) c7(jf, nZ, T, av, eW, A, mx, j, fu, ey, y, bz, bc, cNn); #c1(TMPO) c2(NP_001027455) c3(thymopoietin isoform gamma) c4(30195) c5(30196, 30197, 30198, 30199, 30200, 30201, 30202, 30203, 30204, 30205, 30206, 30207) c6( ) c7(jf, nZ, T, av, eW, A, mx, j, fu, ey, y, bz, bc, cNn); #c1(TMPO) c2(NP_003267) c3(thymopoietin isoform alpha) c4(136945) c5(136946, 136947, 136948, 136949, 136950, 136951) c6( ) c7(jf, nZ, T, av, eW, A, mx, j, fu, ey, y, bz, bc, cNn); #c1(TMPRSS11A) c2(NP_001107859) c3(transmembrane protease serine 11A isoform 2) c4(43040, 43041) c5(43042, 43043, 43044, 43045, 43046, 43047, 43048, 43049, 43050, 43051, 43052, 43053) c6( ) c7(it, ht, ff, en, hu); #c1(TMPRSS11A) c2(NP_872412) c3(transmembrane protease serine 11A isoform 1) c4(256310) c5(256311, 256312, 256313, 256314, 256315, 256316) c6( ) c7(it, ht, ff, en, hu); #c1(TMPRSS11B) c2(NP_872308) c3(transmembrane protease serine 11B) c4(256067) c5(256068, 256069, 256070, 256071, 256072, 256073, 256074, 256075, 256076) c6(256077, 256078) c7(ac, e, ht, aw, j, hu, eu); #c1(TMPRSS11B) c2(XP_011529911) c3(transmembrane protease serine 11B isoform X2) c4(325671) c5(325672, 325673, 325674, 325675, 325676) c6( ) c7(ac, e, ht, aw, j, hu, eu); #c1(TMPRSS11D) c2(NP_004253) c3(transmembrane protease serine 11D) c4(144618, 144619) c5(144620, 144621, 144622, 144623, 144624, 144625, 144626, 144627, 144628) c6(144629, 144630) c7(bM, T, aDu, Jr, aCT, ds, wc, dV, fd, aXd, XZ, ae, eW, ad, DF, hX); #c1(TMPRSS11D) c2(XP_005265767) c3(transmembrane protease serine 11D isoform X1) c4( ) c5(279708, 279709, 279710, 279711, 279712, 279713, 279714) c6(279715, 279716) c7(bM, T, aDu, Jr, aCT, ds, wc, dV, fd, aXd, XZ, ae, eW, ad, DF, hX); #c1(TMPRSS11E) c2(NP_054777) c3(transmembrane protease serine 11E precursor) c4(177750, 177751) c5(177752, 177753, 177754, 177755, 177756, 177757, 177758, 177759, 177760, 177761, 177762, 177763, 177764, 177765) c6(177766, 177767) c7(ik, ff, en, anW); #c1(TMPRSS13) c2(NP_001070731) c3(transmembrane protease serine 13 isoform 1) c4(34815, 34816, 34817) c5(34818, 34819, 34820, 34821, 34822, 34823, 34824, 34825, 34826, 34827, 34828, 34829, 34830, 34831, 34832, 34833, 34834) c6( ) c7(T, qo, Uc, Ol, if, aA, it, j, ci, qN, PJ, fr, q, ad, rc, dP, ft, aE, lg, jZ, pX, dV, aT, bOu, NM, ae, VF, d, cKU, aW, Y, r, lo, aF, xH, g, kk, iK, bz, op, qe, c, eM, dc, vA, agl, DC, uz, ER, eg, Pr, B); #c1(TMPRSS13) c2(NP_001193718) c3(transmembrane protease serine 13 isoform 2) c4(82485) c5(82486, 82487, 82488, 82489, 82490, 82491) c6( ) c7(T, qo, Uc, Ol, if, aA, it, j, ci, qN, PJ, fr, q, ad, rc, dP, ft, aE, lg, jZ, pX, dV, aT, bOu, NM, ae, VF, d, cKU, aW, Y, r, lo, aF, xH, g, kk, ik, bz, op, qe, c, eM, dc, vA, agl, DC, uz, ER, eg, Pr, B); #c1(TMPRSS13) c2(NP_001193719) c3(transmembrane protease serine 13 isoform 3) c4(82492) c5(82493, 82494, 82495, 82496, 82497, 82498) c6( ) c7(T, qo, Uc, Ol, if, aA, it, j, ci, qN, PJ, fr, q, ad, rc, dP, ft, aE, lg, jZ, pX, dV, aT, bOu, NM, ae, VF, d, cKU, aW, Y, r, lo, aF, xH, g, kk, iK, bz, op, qe, c, eM, dc, vA, agl, DC, uz, ER, eg, Pr, B); #c1(TMPRSS13) c2(NP_001231924) c3(transmembrane protease serine 13 isoform 4) c4(87729) c5(87730, 87731, 87732, 87733, 87734, 87735, 87736) c6( ) c7(T, qo, Uc, Ol, if, aA, it, j, ci, qN, PJ, fr, q, ad, rc, dP, ft, aE, lg, jZ, pX, dV, aT, bOu, NM, ae, VF, d, cKU, aW, Y, r, lo, aF, xH, g, kk, iK, bz, op, qe, c, eM, dc, vA, agl, DC, uz, ER, eg, Pr, B); #c1(TMPRSS15) c2(NP_002763) c3 (enteropeptidase precursor) c4(131790) c5(131791, 131792, 131793, 131794, 131795, 131796) c6( ) c7(T, EQ, v, cNo, ci, g); #c1(TMPRSS15) c2(XP_011527958) c3(enteropeptidase isoform X1) c4( ) c5(323018, 323019) c6( ) c7(T, EQ, v, cNo, ci, g); #c1(TMPRSS2) c2(NP_001128571) c3(transmembrane protease serine 2 isoform 1) c4(50282, 50283, 50284) c5(50285, 50286, 50287, 50288, 50289, 50290, 50291, 50292, 50293, 50294) c6( ) c7(gh, G, T, dA, fZ, n, bnS, Fb, ci, ad, ft, ds, ae, il, NE, qt, AQ, wc, g, GU, np, bt); #c1(TMPRSS2) c2(XP_005261100) c3(transmembrane protease serine 2 isoform X3) c4(277211) c5( ) c6( ) c7(gh, G, T, dA, fZ, n, bnS, Fb, ci, ad, ft, ds, ae, iL, NE, qt, AQ, wc, g, GU, np, bt); #c1(TMPRSS2) c2(XP_011528033) c3(transmembrane protease serine 2 isoform X1) c4( ) c5(323119, 323120, 323121, 323122, 323123, 323124, 323125, 323126, 323127, 323128, 323129, 323130, 323131) c6( ) c7(gh, G, T, dA, fZ, n, bnS, Fb, ci, ad, ft, ds, ae, iU, NE, qt, AQ, wc, g, GU, np, bt); #c1(TMPRSS2) c2(XP_011528034) c3 (transmembrane protease serine 2 isoform X2) c4( ) c5(323132, 323133, 323134, 323135, 323136) c6( ) c7(gh, G, T, dA, fZ, n, bnS, Fb, ci, ad, ft, ds, ae, il, NE, qt, AQ, wc, g, GU, np, bt); #c1(TMPRSS3) c2(NP_001243246) c3(transmembrane protease serine 3 isoform 4) c4(90245) c5(90246, 90247, 90248, 90249, 90250, 90251) c6( ) c7(cZ, gJ, T, aE, agT, dd, mG, alA, cNq, yU, yZ, m, OM, t, cNp, Bh, bCJ); #c1(TMPRSS3) c2(NP_076927) c3(transmembrane protease serine 3 isoform 1) c4(214287, 214288) c5(214289, 214290, 214291, 214292, 214293, 214294, 214295, 214296, 214297, 214298, 214299, 214300) c6( ) c7(cZ, gJ, T, aE, agT, dd, mG, alA, cNq, yU, yZ, m, OM, t, cNp, Bh, bCJ); #c1(TMPRSS3) c2(NP_115781) c3(transmembrane protease serine 3 isoform 3) c4(223874) c5(223875, 223876, 223877, 223878, 223879, 223880) c6( ) c7(cZ, gJ, T, aE, agT, dd, mG, alA, cNq, yU, yZ, m, OM, t, cNp, Bh, bCJ); #c1(TMPRSS4) c2(NP_001077416) c3(transmembrane protease serine 4 isoform 3) c4(37828) c5(37829, 37830, 37831, 37832, 37833, 37834) c6( ) c7(cZ, T, aw, aA, bB, q, av, hd, aE, io, cNq, hB, dd, ff, r, aF, en, g, t, bz, eM, nB, bE, alA, m, Bh); #c1(TMPRSS4) c2(NP_001167022) c3(transmembrane protease serine 4 isoform 4) c4(68881) c5(68882, 68883, 68884, 68885, 68886, 68887) c6( ) c7(cZ, T, aw, aA, bB, q, av, hd, aE, io, cNq, hB, dd, ff, r, aF, en, g, t, bz, eM, nB, bE, alA, m, Bh); #c1(TMPRSS4) c2(NP_001167023) c3(transmembrane protease serine 4 isoform 5) c4(68888) c5(68889, 68890, 68891, 68892, 68893, 68894) c6( ) c7(cZ, T, aw, aA, bB, q, av, hd, aE, io, cNq, hB, dd, ff, r, aF, en, g, t, bz, eM, nB, bE, alA, m, Bh); #c1(TMPRSS4) c2(NP_063947) c3(transmembrane protease serine 4 isoform 1) c4(202394) c5(202395, 202396, 202397, 202398, 202399, 202400) c6( ) c7(cZ, T, aw, aA, bB, q, av, hd, aE, io, cNq, hB, dd, ff, r, aF, en, g, t, bz, eM, nB, bE, alA, m, Bh); #c1(TMPRSS6) c2(NP_705837) c3(transmembrane protease serine 6 isoform 2) c4(246303, 246304, 246305) c5(246306, 246307, 246308, 246309, 246310, 246311, 246312, 246313, 246314, 246315, 246316, 246317, 246318) c6( ) c7(bL, T, kP, yw, pN, oz, awC, aUW, aW, oW, aT, cz, yu, yy, K, pw, an, Rc, qD, ae, ad); #c1(TMPRSS6) c2(XP_006724225) c3 (transmembrane protease serine 6 isoform X2) c4(295851) c5(295852, 295853, 295854, 295855, 295856, 295857, 295858, 295859, 295860, 295861, 295862) c6( ) c7(bL, T, kP, yw, pN, oz, awC, aUW, aW, oW, aT, cz, yu, yy, K, pw, an, Rc, qD, ae, ad); #c1(TMPRSS6) c2(XP_011528290) c3(transmembrane protease serine 6 isoform X1) c4(323355) c5(323356, 323357, 323358, 323359, 323360, 323361, 323362, 323363, 323364, 323365) c6( ) c7(bL, T, kP, yw, pN, oz, awC, aUW, aW, oW, aT, cz, yu, yy, K, pw, an, Rc, qD, ae, ad); #c1(TMPRSS7) c2(NP_001036040) c3(transmembrane protease serine 7) c4(33808) c5(33809, 33810, 33811, 33812, 33813) c6( ) c7(IB); #c1(TMPRSS7) c2(XP_011511057) c3(transmembrane protease serine 7 isoform X1) c4(301471, 301472, 301473) c5(301474, 301475, 301476, 301477, 301478, 301479, 301480, 301481, 301482, 301483, 301484, 301485, 301486, 301487, 301488, 301489, 301490, 301491, 301492) c6( ) c7(IB); #c1(TMPRSS9) c2(NP_892018) c3(transmembrane protease serine 9) c4(257625) c5(257626, 257627, 257628, 257629, 257630, 257631) c6( ) c7(pH); #c1(TMPRSS9) c2(XP_011526285) c3(transmembrane protease serine 9 isoform X6) c4(320547) c5(320548, 320549, 320550, 320551, 320552, 320553) c6( ) c7(pH); #c1(TMSB10) c2(NP_066926) c3(thymosin beta-10) c4(207522) c5(207523, 207524, 207525, 207526, 207527, 207528) c6( ) c7(T, dD, ea, eq, ci, av, hd, jz, aE, aT, Fx, K, bK, hu, Y, fJ, cm, t, nM, Dy, ig, m); #c1(TMSB15B) c2(NP_919305) c3(thymosin beta-15B) c4(258522) c5(258523, 258524, 258525, 258526, 258527, 258528, 258529, 258530, 258531) c6( ) c7(kP, bE, bB, h, cm, ae, ad); #c1(TMSB4X) c2(NP_066932) c3(thymosin beta-4) c4(207545, 207546) c5(207547, 207548, 207549, 207550, 207551, 207552, 207553, 207554, 207555, 207556, 207557, 207558, 207559, 207560, 207561, 207562, 207563, 207564, 207565, 207566, 207567, 207568, 207569, 207570, 207571, 207572, 207573, 207574, 207575) c6(207576, 207577, 207578, 207579) c7(T, kh, aCA, aC, id, bfx, rf, aA, bB, fd, j, ci, fr, PJ, q, ad, eA, x, F, aE, K, bq, vx, hm, yF, le, eG, d, Y, r, aF, eM, bE, bsO, bm, k, hE); #c1(TMTC1) c2(NP_001180380) c3(transmembrane and TPR repeat-containing protein 1 isoform 1) c4(73892) c5(73893, 73894, 73895, 73896, 73897, 73898, 73899) c6( ) c7(he, fq, bK, Zt); #c1(TMTC1) c2(NP_787057) c3(transmembrane and TPR repeat-containing protein 1 isoform 2) c4(252033) c5(252034, 252035, 252036, 252037, 252038) c6( ) c7(he, fq, bK, Zt); #c1(TMTC1) c2(XP_005253556) c3(transmembrane and TPR repeat-containing protein 1 isoform X4) c4(272551, 272552, 272553) c5(272554, 272555, 272556, 272557, 272558, 272559, 272560, 272561, 272562, 272563, 272564, 272565, 272566, 272567, 272568, 272569, 272570, 272571) c6( ) c7(he, fq, bK, Zt); #c1(TMTC2) c2(NP_689801) c3(transmembrane and TPR repeat-containing protein 2 precursor) c4(243995) c5(243996, 243997, 243998, 243999, 244000, 244001) c6( ) c7(fe, fq, ad, pZ); #c1(TMTC3) c2(NP_861448) c3(transmembrane and TPR repeat-containing protein 3) c4(255587, 255588) c5(255589, 255590, 255591, 255592, 255593, 255594, 255595, 255596, 255597, 255598, 255599, 255600) c6( ) c7(gV, jh, hG); #c1(TMX1) c2(NP_110382) c3(thioredoxin-related transmembrane protein 1 precursor) c4(219834) c5(219835, 219836, 219837, 219838, 219839, 219840) c6( ) c7(ey, eZ); #c1(TMX2) c2(NP_001137484) c3(thioredoxin-related transmembrane protein 2 isoform 2) c4(55928) c5(55929, 55930, 55931, 55932, 55933, 55934) c6( ) c7(aW, aT); #c1(TMX2) c2(NP_057043) c3(thioredoxin-related transmembrane protein 2 isoform 1 precursor) c4(188613) c5(188614, 188615, 188616, 188617, 188618, 188619) c6( ) c7(aW, al); #c1(TMX3) c2(NP_061895) c3(protein disulfide-isomerase TMX3 precursor) c4(201017, 201018) c5(201019, 201020, 201021, 201022, 201023, 201024, 201025, 201026, 201027, 201028) c6( ) c7(kn, kj); #c1(TMX3) c2(XP_011524334) c3 (protein disulfide-isomerase TMX3 isoform X1) c4(319002) c5(319003, 319004, 319005, 319006, 319007, 319008, 319009, 319010) c6( ) c7(kn, kj); #c1(TNC) c2(NP_002151) c3(tenascin precursor) c4(125974) c5(125975, 125976, 125977, 125978, 125979, 125980, 125981, 125982, 125983, 125984, 125985, 125986, 125987, 125988, 125989, 125990) c6( ) c7(ID, bM, T, ea, if, e, al, bB, lR, ci, Hr, ip, Na, aE, Bc, rn, fm, pX, K, lQ, eL, ly, eG, aW, Ls, g, as, eE, zE, eF, cm, eM, nB, wB, vy, Hb, m, fy, dD, pG, aw, qy, n, fr, cd, ai, Mo, Bl, jz, h, mJ, dx, wC, aiB, ks, kp, jT, cM, lx, dB, hk, ajd, nz, it, vl, cNs, dA, fG, Lx, fZ, v, j, AO, Eh, q, eA, bag, nv, aT, Nc, Fx, zp, bq, ae, jf, bf, n, Y, en, cNr, t, iK, pb, ck, cT, rR, cNt, all, ro, rA, Bh, awa, hS, kh, Dd, Fb, eq, SY, aFJ, ad, qC, fU, aHF, hd, dP, ib, av, iC, Ot, hP, lQ, ul, d, qt, ff, eH, cr, aiz, si, bz, cNu, LB, Ew, zr, bE, z, gZ, aS, adg, lC, y); #c1(TNC) c2(XP_005252031) c3(tenascin isoform X16) c4(271468) c5(271469, 271470, 271471, 271472, 271473, 271474, 271475, 271476) c6( ) c7(ID, bM, T, ea, if, e, al, bB, lR, ci, Hr, ip, Na, aE, Bc, rn, fm, pX, K, lQ, eL, ly, eG, aW, Ls, g, as, eE, ZE, eF, cm, eM, nB, wB, vy, Hb, m, fy, dD, pG, aw, qy, n, fr, cd, ai, Mo, Bl, jz, h, mJ, dx, wC, aiB, ks, kp, jT, cM, lx, dB, hk, ajd, nz, it, vQ, cNs, dA, fG, Lx, fZ, v, j, AO, Eh, q, eA, bag, nv, aT, Nc, Fx, zp, bq, ae, jf, bf, nO, Y, en, cNr, t, ik, pb, ck, cT, rR, cNt, all, ro, rA, Bh, awa, hS, kh, Dd, Fb, eq, SY, aFJ, ad, qC, fU, aHF, hd, dP, ib, av, iC, Ot, hP, lQ, ul, d, qt, ff, eH, cr, aiz, si, bz, cNu, LB, Ew, zr, bE, z, gZ, aS, adg, lC, y); #c1(TNC) c2(XP_005252032) c3(tenascin isoform X18) c4(271477, 271478) c5(271479, 271480, 271481, 271482, 271483, 271484, 271485, 271486, 271487, 271488, 271489, 271490, 271491, 271492, 271493) c6(271494) c7(ID, bM, T, ea, if, e, al, bB, lR, ci, Hr, ip, Na, aE, Bc, rn, fm, pX, K, lQ, eL, ly, eG, aW, Ls, g, as, eE, zE, eF, cm, eM, nB, wB, vy, Hb, m, fy, dD, pG, aw, qy, n, fr, cd, ai, Mo, Bl, jz, h, mJ, dx, wC, aiB, ks, kp, jT, cM, lx, dB, hk, ajd, nz, it, vl, cNs, dA, fG, Lx, fZ, v, j, AO, Eh, q, eA, bag, nv, aT, Nc, Fx, zp, bq, ae, jf, bf, nO, Y, en, cNr, t, iK, pb, ck, cT, rR, cNt, all, ro, rA, Bh, awa, hS, kh, Dd, Fb, eq, SY, aFJ, ad, qC, fU, aHF, hd, dP, ib, av, iC, Ot, hP, lQ, ul, d, qt, ff, eH, cr, aiz, si, bz, cNu, LB, Ew, zr, bE, z, gZ, aS, adg, lC, y); #c1(TNC) c2(XP_006717159) c3(tenascin isoform X1) c4(290783) c5( ) c6( ) c7(ID, bM, T, ea, if, e, al, bB, lR, ci, Hr, ip, Na, aE, Bc, rn, fm, pX, K, lQ, eL, ly, eG, aW, Ls, g, as, eE, zE, eF, cm, eM, nB, wB, vy, Hb, m, fy, dD, pG, aw, qy, n, fr, cd, ai, Mo, Bl, jz, h, mJ, dx, wC, aiB, ks, kp, jT, cM, lx, dB, hk, ajd, nz, it, vQ, cNs, dA, fG, Lx, fZ, v, j, AO, Eh, q, eA, bag, nv, aT, Nc, Fx, zp, bq, ae, jf, bf, nO, Y, en, cNr, t, ik, pb, ck, cT, rR, cNt, all, ro, rA, Bh, awa, hS, kh, Dd, Fb, eq, SY, aFJ, ad, qC, fU, aHF, hd, dP, ib, av, iC, Ot, hP, lQ, uQ, d, qt, ff, eH, cr, aiz, si, bz, cNu, LB, Ew, zr, bE, z, gZ, aS, adg, lC, y); #c1(TNC) c2(XP_011516929) c3(tenascin isoform X11) c4(310441, 310442) c5(310443, 310444, 310445, 310446, 310447, 310448, 310449, 310450, 310451, 310452, 310453, 310454, 310455, 310456, 310457, 310458) c6( ) c7(ID, bM, T, ea, if, e, al, bB, lR, ci, Hr, ip, Na, aE, Bc, rn, fm, pX, K, lQ, eL, ly, eG, aW, Ls, g, as, eE, ZE, eF, cm, eM, nB, wB, vy, Hb, m, fy, dD, pG, aw, qy, n, fr, cd, ai, Mo, Bl, jz, h, mJ, dx, wC, aiB, ks, kp, jT, cM, lx, dB, hk, ajd, nz, it, vQ, cNs, dA, fG, Lx, fZ, v, j, AO, Eh, q, eA, bag, nv, aT, Nc, Fx, zp, bq, ae, jf, bf, nO, Y, en, cNr, t, ik, pb, ck, cT, rR, cNt, all, ro, rA, Bh, awa, hS, kh, Dd, Fb, eq, SY, aFJ, ad, qC, fU, aHF, hd, dP, ib, av, iC, Ot, hP, lQ, ul, d, qt, ff, eH, cr, aiz, si, bz, cNu, LB, Ew, zr, bE, z, gZ, aS, adg, lC, y); #c1(TNC) c2(XP_011516931) c3(tenascin isoform X14) c4(310459) c5(310460, 310461, 310462, 310463, 310464, 310465, 310466, 310467) c6( ) c7(ID, bM, T, ea, if, e, al, bB, lR, ci, Hr, ip, Na, aE, Bc, rn, fm, pX, K, lQ, eL, ly, eG, aW, Ls, g, as, eE, ZE, eF, cm, eM, nB, wB, vy, Hb, m, fy, dD, pG, aw, qy, n, fr, cd, ai, Mo, Bl, jz, h, mJ, dx, wC, aiB, ks, kp, jT, cM, lx, dB, hk, ajd, nz, it, vl, cNs, dA, fG, Lx, fZ, v, j, AO, Eh, q, eA, bag, nv, aT, Nc, Fx, zp, bq, ae, jf, bf, nO, Y, en, cNr, t, ik, pb, ck, cT, rR, cNt, all, ro, rA, Bh, awa, hS, kh, Dd, Fb, eq, SY, aFJ, ad, qC, fU, aHF, hd, dP, ib, av, iC, Ot, hP, lQ, uQ, d, qt, ff, eH, cr, aiz, si, bz, cNu, LB, Ew, zr, bE, z, gZ, aS, adg, lC, y); #c1(TNF) c2(NP_000585) c3(tumor necrosis factor) c4(13225, 13226) c5(13227, 13228, 13229, 13230, 13231, 13232, 13233, 13234, 13235, 13236, 13237, 13238, 13239, 13240, 13241, 13242, 13243, 13244, 13245, 13246, 13247, 13248, 13249, 13250, 13251, 13252, 13253, 13254, 13255, 13256) c6( ) c7(uB, gh, T, os, ea, bO, fd, Vd, ci, aY, blt, aDl, bv, bQB, xy, aE, rw, bJj, dV, bbu, Ds, Bi, hu, zy, hH, LG, aiJ, cp, VF, aa, ly, azh, hC, az, nY, uY, dg, vs, tU, lk, GR, zV, axG, ba, fT, gt, Jr, nB, dc, iA, wE, Ji, cj, afp, cNC, Zn, Dt, k, bAO, aZo, azg, fy, lS, vF, eg, aEw, LT, cE, nD, aYZ, bPN, ht, xJ, aj, PJ, bPL, qW, cGB, br, tD, cd, zo, brn, Wy, aoc, aSa, lG, Gw, aNj, ey, ox, bl, bEg, NX, pl, mR, anP, sp, xi, ge, fQ, sm, bNY, fe, aMJ, CQ, adz, hq, vU, iO, ok, RB, dB, Fi, eD, bOj, Ei, ajd, aoB, E, aqm, abW, DM, jv, aZy, DD, aA, cz, bX, wy, nS, q, Wu, zn, av, aWb, nH, aCX, yu, Di, cNB, cy, aAO, jj, rz, sX, caR, dl, bA, eu, le, aFS, bf, nO, fO, lC, bg, aMA, iy, lo, bRQ, cl, Vk, pb, Cv, ov, cT, rR, OW, ua, ac, ig, iM, Nk, il, mG, AU, tz, pS, bt, Ze, blz, ao, rA, gV, oq, Ve, acv, arS, axd, uG, iW, jh, aZH, oZ, dG, Sv, fu, uu, aYs, ad, wD, aoH, hX, HF, yt, vJ, Ot, oW, IQ, wm, cNG, bPM, aAy, WZ, cNH, lx, ZE, cH, cW, ff, Of, aLl, nh, afi, Mz, cxe, dq, cNM, py, EZ, Sm, aFd, iV, bz, aZz, aYt, DF, aJx, amK, ok, aDH, Om, anY, ag, aDB, cmZ, aDJ, ME, aEN, oQ, afT, Pk, lC, lf, ne, rk, iu, bsJ, oO, bM, lD, qo, vS, bQl, xS, if, e, bB, cL, gA, ur, auu, bh, sD, ajC, Tz, ip, yM, x, vV, fS, bFU, Yy, ja, us, azm, bc, akX, bQs, eG, up, YY, Jl, dH, fc, hl, kt, pD, NR, sz, aFU, as, eE, eF, lt, cm, bQF, bw, eY, aqr, lt, bQl, xD, ii, ki, vA, aGe, mx, vy, bcl, Dv, pJ, aFf, Vj, abh, avY, xN, dD, gU, bJs, aBe, Ht, wu, zt, yG, n, aCZ, uE, Mo, jz, no, pe, ec, zw, RN, Xe, ccy, bRM, eV, hn, bQx, cY, xP, A, fJ, lb, aeU, hQ, QP, po, yq, amo, bQL, vo, cNl, jq, bP, cA, aZR, Pl, ajn, hl, at, apY, bPs, awd, ut, cNE, fl, eo, cB, B, qc, vQ, pF, dA, wL, lH, eX, YN, fF, GC, aNG, v, Qo, aFn, V, eJ, Yn, bQG, yw, bjv, bjA, WY, cNy, sl, ld, bRt, rS, aDt, azv, XZ, rE, ae, to, blm, aFs, hM, jf, zH, iZ, vR, ev, DN, aLJ, afq, HL, acB, bu, t, cul, aff, vk, aHG, ck, kP, wl, gX, hO, auT, oX, yH, byK, vi, HQ, hl, ra, Yu, MH, dM, df, OR, wV, eq, qi, Fc, aGW, BY, hd, CJ, cNP, od, afm, Db, alz, ds, aCJ, Hk, qh, hD, rN, JK, nJ, sj, d, je, hG, KS, wc, aFt, cNw, dN, th, anW, Z, Hv, dr, cND, aDN, bF, D, gk, yE, Ny, dO, bQS, lI, wz, gZ, aef, MJ, gy, Hs, rH, agl, ajt, y, kS, cNO, sH, hv, pC, cNA, aC, cNO, FR, hV, azi, zs, atj, al, CH, axk, gf, qj, aGX, lR, aBm, Qp, aMB, wa, bOm, AW, bOn, wM, xE, bJG, u, Bc, rJ, pX, bW, bJK, M, alr, K, wt, DY, dF, cnS, N, mH, bzH, eL, aEV, aCM, bn, aAg, aAT, qS, asD, mQ, aGQ, axu, wf, hN, aB, g, pl, ve, uC, iP, bRl, aJX, aik, yl, xK, aDs, AM, uy, aer, Gs, U, uo, dW, Hb, m, abX, bJv, NK, FW, sQ, aik, sw, gp, aw, fN, BH, NJ, BF, apt, aBl, pM, aCQ, pU, eh, fK, asS, ft, kH, Lv, cU, Ne, nm, wb, uV, ZN, aDp, Ha, dx, aXy, arp, Ni, alw, ks, Og, aGq, YF, jT, cM, Zb, Ld, Zj, bMc, kT, ar, GG, W, SE, bbx, xO, wk, bo, mg, JM, Br, aiS, gg, lB, Pr, it, L, ayV, ach, aAf, fM, AL, wj, fG, jx, aee, wo, zN, uZ, bJw, j, hz, azF, sl, aYW, il, pd, eA, J, o, cNx, bJO, bJn, hb, nX, jZ, apJ, ceS, aCV, bq, bPQ, aCr, azU, kl, PK, xR, CNN, Ue, Y, Pg, apK, r, wl, aHU, cNF, en, wA, aoG, iK, bN, QG, sn, c, Dy, and, hA, hc, aDA, of, dh, tM, uS, caP, wR, Da, rK, dy, EY, fD, bxj, gT, LS, blu, gx, sY, kh, Gl, aFe, bml, am, Pt, aZ, LX, em, id, bs, awz, bS, auq, SY, ah, iG, pl, hF, gb, iJ, dP, fU, ceL, fx, sN, NY, clq, oj, io, vB, aNp, bNL, hP, aSf, aOh, ji, NM, WW, uX, lU, BN, lR, anv, anX, aZl, eH, Sn, cr, si, hp, Fq, Pd, iR, YZ, KM, qa, dR, Nq, TA, Bv, Lj, afb, eB, sG, aEs, aCl, cP, ei, Gu, hE, vt, xg, bol, bJx, pf, eb, zh, Jx, mF, fw, bMW, aR, bHS, dY, aDk, il, iX, bOL, bAy, aYm, qM, bU, oi, H, dQ, eN, aWf, pk, hm, akV, zQ, bLs, aAu, aW, aEH, lh, Rr, aOW, bdk, aRS, hJ, arZ, cNJ, eM, iT, Jp, qr, aqV, uJ, mk, gn, R, pw, aSs, qR, gD, aEj, My, bmW, ez, zj, Mi, ct, jG, qN, fr, kO, aPb, ai, eW, xC, ke, Bl, cc, hw, qg, wG, ql, kl, ju, zf, h, jy, Nf, aPe, dL, bam, pj, hB, vx, bnv, afu, WC, Nr, PT, OV, asp, oo, bVg, qY, aq, kp, mo, gG, dw, acD, op, lx, OU, Wt, ZL, Qy, vD, bpC, NL, hk, uw, Qu, bm, uz, va, aAl, aDE, bai, bni, bD, bL, Ob, Dw, lk, eR, Wz, dk, uv, yk, DV, rf, sM, CR, tc, bak, hy, OY, C, aoC, alx, ady, cF, ga, GL, mP, uT, Vr, qp, afQ, HC, aT, dz, bK, adV, UN, cNz, an, KU, af, aZQ, aQk, ND, iU, oV, aNi, akY, aDm, lA, kC, cNK, f, DC, wf, lY, ajl, akF, sS, wg, nM, pZ, Zg, vZ, lV, ahN, AH, fP, aGV, mf, CZ, bko, im, hS, Kd, qX, Ke, qb, aD, Me, oH, jE, pz, cNL, mC, Uy, cNv, LH, qC, XB, dZ, l, F, ot, kL, xl, oz, sO, NS, azq, Os, vw, jo, aFl, vD, yF, ul, bwk, Di, aFE, eZ, yy, Ej, aFr, xv, aF, gu, Tp, eK, Hp, LB, es, eL, axs, atM, bE, pE, Rv, z, uR, arj, aS, aGg, be, Lz, pK, XO, fh, X, Ex); #c1(TNFAIP1) c2(NP_066960) c3(BTB/POZ domain-containing adapter for CUL3-mediated RhoA degradation protein 2) c4(207784, 207785) c5(207786, 207787, 207788, 207789, 207790, 207791, 207792, 207793, 207794, 207795, 207796, 207797) c6( ) c7(bM, ac, aW, eZ, aT, v, my, ey, aF, y, af, ul); #c1(TNFAIP2) c2(NP_006282) c3(tumor necrosis factor alpha-induced protein 2) c4(163565, 163566, 163567) c5(163568, 163569, 163570, 163571, 163572, 163573, 163574, 163575, 163576, 163577, 163578, 163579, 163580, 163581, 163582, 163583) c6( ) c7(Gl, T, eM, if, ht, aA, By, hy, j, aF, qN, Gu, Gw, t, iK, jj, af); #c1(TNFAIP2) c2(XP_011535415) c3(tumor necrosis factor alpha-induced protein 2 isoform X2) c4(332448) c5(332449, 332450, 332451, 332452, 332453, 332454, 332455, 332456, 332457, 332458, 332459) c6( ) c7(Gl, T, eM, if, ht, aA, By, hy, j, aF, qN, Gu, Gw, t, iK, j, af); #c1(TNFAIP2) c2(XP_011535417) c3(tumor necrosis factor alpha-induced protein 2 isoform X3) c4( ) c5(332460) c6( ) c7(Gl, T, eM, if, ht, aA, By, hy, j, aF, qN, Gu, Gw, t, iK, jj, af); #c1(TNFAIP3) c2(NP_006281) c3 (tumor necrosis factor alpha-induced protein 3) c4(163539, 163540, 163541, 163542, 163543, 163544, 163545, 163546) c5(163547, 163548, 163549, 163550, 163551, 163552, 163553, 163554, 163555, 163556, 163557, 163558, 163559, 163560, 163561, 163562, 163563, 163564) c6( ) c7(bL, bM, T, dD, fM, ex, fN, jh, cl, j, eq, uE, V, q, Uy, o, ad, ai, wD, eV, hn, hH, af, hM, A, d, mR, HA, cr, ge, r, eZ, fe, hp, iV, t, cM, ve, pb, eL, zr, JM, cj, yH, ij, y); #c1(TNFAIP3) c2(XP_011534398) c3(tumor necrosis factor alpha-induced protein 3 isoform X2) c4(330784) c5(330785, 330786, 330787, 330788, 330789, 330790, 330791, 330792, 330793, 330794, 330795, 330796, 330797, 330798, 330799, 330800, 330801, 330802, 330803, 330804, 330805, 330806, 330807, 330808, 330809, 330810, 330811, 330812, 330813, 330814, 330815, 330816, 330817, 330818, 330819, 330820) c6( ) c7(bL, bM, T, dD, fM, eX, fN, jh, cl, j, eq, uE, V, q, Uy, o, ad, ai, wD, eV, hn, hH, af, hM, A, d, mR, HA, cr, ge, r, eZ, fe, hp, iV, t, cM, ve, pb, eL, zr, JM, cj, yH, ij, y); #c1(TNFAIP6) c2(NP_009046) c3(tumor necrosis factor-inducible gene 6 protein precursor) c4(170407) c5(170408, 170409, 170410, 170411, 170412, 170413) c6( ) c7(ai, T, wi, ds, fJ, JM, r, hN, lQ, Lw, t, Pk, cM, q, ad, pl, aa); #c1(TNFAIP8) c2(NP_001071122) c3(tumor necrosis factor alpha-induced protein 8 isoform b) c4(35301) c5(35302, 35303, 35304, 35305, 35306, 35307) c6( ) c7(T, if, oe, jh, bB, n, fr, jE, qN, ad, aT, ey, ae, aW, g, iK, bz, eM, iA, bE, m, it); #c1(TNFAIP8) c2(NP_001273742) c3(tumor necrosis factor alpha-induced protein 8 isoform a) c4(105744) c5(105745, 105746, 105747, 105748, 105749, 105750, 105751, 105752, 105753, 105754) c6( ) c7(T, if, oe, jh, bB, n, fr, jE, qN, ad, aT, ey, ae, aW, g, iK, bz, eM, iA, bE, m, it); #c1(TNFAIP8L2) c2(NP_078851) c3(tumor necrosis factor alpha-induced protein 8-like protein 2) c4(215752) c5(215753, 215754, 215755, 215756, 215757, 215758) c6( ) c7(Kd, hE, jE, eZ, hX); #c1(TNFAIP8L3) c2(NP_997264) c3(tumor necrosis factor alpha-induced protein 8-like protein 3) c4(265353) c5(265354, 265355, 265356, 265357, 265358, 265359) c6( ) c7(T); #c1(TNFRSF1DA) c2(NP_003835) c3(tumor necrosis factor receptor superfamily member 10A) c4(141246) c5(141247, 141248, 141249, 141250, 141251, 141252) c6( ) c7(gV, T, dD, fM, fR, pf, ht, jx, qy, aA, bB, fd, jh, eq, ci, bX, fr, q, ad, eA, wD, cF, F, ib, ec, fS, oW, aT, h, cNR, ja, dL, DU, jj, ae, eL, jf, A, d, B, s, je, aW, Y, ff, HA, hl, eH, r, aq, lo, aF, en, g, as, t, eF, bz, ik, iR, bN, ck, bF, iM, W, bE, eD, cj, at, aeM, m, y, bt, X); #c1(TNFRSF10B) c2(NP_003833) c3(tumor necrosis factor receptor superfamily member 10B isoform 1 precursor) c4(141239) c5(141240, 141241, 141242, 141243, 141244, 141245) c6( ) c7(bL, bM, T, aC, fM, fR, jp, e, jh, aA, bB, fd, fr, ci, q, ad, eA, eW, XX, bv, av, ib, aE, gd, aT, h, cNR, dL, K, ae, eL, cY, d, B, s, je, aW, Y, eH, cr, r, eZ, aq, lo, kp, aF, g, kk, t, eF, bz, ik, ck, bF, eL, iM, kP, eM, dB, bE, eD, AH, cj, m, y, bt, X, vi); #c1(TNFRSF10B) c2(NP_671716) c3(tumor necrosis factor receptor superfamily member 10B isoform 2 precursor) c4(242146, 242147) c5(242148, 242149, 242150, 242151, 242152, 242153, 242154, 242155, 242156, 242157, 242158, 242159) c6( ) c7(bL, bM, T, aC, fM, fR, jp, e, jh, aA, bB, fd, fr, ci, q, ad, eA, eW, XX, bv, av, ib, aE, gd, aT, h, cNR, dL, K, ae, eL, cY, d, B, s, je, aW, Y, eH, cr, r, eZ, aq, lo, kp, aF, g, kk, t, eF, bz, ik, ck, bF, eL, iM, kP, eM, dB, bE, eD, AH, cj, m, y, bt, X, vi); #c1(TNFRSF10C) c2(NP_003832) c3(tumor necrosis factor receptor superfamily member 10C precursor) c4(141218, 141219, 141220) c5(141221, 141222, 141223, 141224, 141225, 141226, 141227, 141228, 141229, 141230, 141231, 141232, 141233, 141234, 141235, 141236, 141237, 141238) c6( ) c7(gV, T, fd, bB, amj, aoj, nA, eA, ad, anj, jz, aE, ec, aT, K, jj, MO, ae, Nr, hG, Y, eZ, po, g, f, t, cm, Nq, EW, bE, m, bt, B); #c1(TNFRSF10D) c2(NP_003831) c3(tumor necrosis factor receptor superfamily member 100 precursor) c4(141211) c5(141212, 141213, 141214, 141215, 141216, 141217) c6( ) c7(T, fR, aAz, fd, bB, hy, ci, eA, ad, aE, ec, aT, jj, ae, BN, aW, Y, aF, cm, bz, eM, bE, y, bt, ne); #c1(TNFRSF11A) c2(NP_001257880) c3(tumor necrosis factor receptor superfamily member 11A isoform 4 precursor) c4(95729) c5(95730, 95731, 95732, 95733, 95734, 95735) c6( ) c7(oO, T, kh, dD, mr, ea, zs, lH, e, zt, fd, cl, v, j, eq, wV, ai, o, ad, eA, cNT, zw, ds, rS, yg, aT, dV, bdM, ae, af, cNV, hm, bA, A, d, aW, Y, ff, eH, cNS, eZ, cNU, en, as, pl, eF, iV, bz, D, eM, yE, cj, uz, lS, fD, bi, zu, bt, Ze, y); #c1(TNFRSF11A) c2(NP_003830) c3(tumor necrosis factor receptor superfamily member 11A isoform 1 precursor) c4(141200) c5(141201, 141202, 141203, 141204, 141205, 141206, 141207, 141208, 141209, 141210) c6( ) c7(oO, T, kh, dD, mr, ea, zs, lH, e, zt, fd, cl, v, j, eq, wV, ai, o, ad, BA, cNT, zw, ds, rS, yg, aT, dV, bdM, ae, af, cNV, hm, bA, A, d, aW, Y, ff, eH, cNS, eZ, cNU, en, as, pl, eF, iV, bz, D, eM, yE, cj, uz, lS, fD, bi, zu, bt, Ze, y); #c1(TNFRSF11B) c2(NP_002537) c3(tumor necrosis factor receptor superfamily member 11B precursor) c4(129635) c5(129636, 129637, 129638, 129639, 129640, 129641) c6( ) c7(oO, T, ea, zs, e, al, fd, cl, zh, ci, cNX, Tz, aE, pX, M, aZn, K, dd, azh, hm, aW, Np, hl, pl, eF, zV, ba, aGl, rq, eM, mx, azg, bi, dD, Ly, FG, zt, aw, zj, n, aj, fr, cd, ai, zw, lT, rC, zf, aFq, adX, awV, A, mR, Hz, aq, kp, pm, vU, adZ, bm, at, uz, pg, B, vQ, bL, dA, eR, mr, De, lH, aA, cz, wo, v, j, V, q, J, o, eA, uT, rS, yg, aT, dz, ae, af, eu, bA, aRR, bf, fO, bg, aeS, r, cNW, akB, lo, rs, pa, iM, tW, ac, hc, cNt, cS, gT, zu, hl, bQb, vr, wV, dG, eq, azk, ad, dP, fU, abk, av, F, oW, bdM, iY, uX, je, vj, eH, eZ, cNU, aF, iR, HI, aZz, D, gk, yE, aS, ei, y); #c1(TNFRSF12A) c2(NP_057723) c3(tumor necrosis factor receptor superfamily member 12A precursor) c4(192505, 192506) c5(192507, 192508, 192509, 192510, 192511, 192512, 192513, 192514, 192515, 192516, 192517, 192518) c6( ) c7(bL, gh, T, dD, fM, hl, id, pf, ea, dk, aw, uG, aA, fd, n, v, eq, Fb, uE, V, eA, ad, hX, sN, hb, Dq, M, aT, cy, bl, K, ji, dQ, ae, Us, d, lR, aW, Y, lb, aq, aF, g, cl, iR, t, KM, bz, ek, ck, D, ac, eM, dB, wD, WP, mx, hL, E, B); #c1(TNFRSF13B) c2(NP_036584) c3(tumor necrosis factor receptor superfamily member 13B) c4(174529, 174530) c5(174531, 174532, 174533, 174534, 174535, 174536, 174537, 174538, 174539, 174540, 174541, 174542) c6( ) c7(oO, bM, T, dA, jh, fd, aFF, eA, di, bjA, afm, ds, ZA, DY, clb, ul, cla, oV, ff, lC, cr, aw, eZ, afq, en, cNY, cNZ, Wt, asE, ck, lY, ac, zr, dJ, of, eB, cj, tr, fl, ei, bD, y, Ze, us); #c1(TNFRSF13C) c2(NP_443177) c3(tumor necrosis factor receptor superfamily member 13C) c4(229727) c5(229728, 229729, 229730, 229731, 229732, 229733) c6( ) c7(T, fM, iW, cOc, eX, jh, fd, V, mC, zn, x, ec, DY, eG, eH, cr, eZ, eF, Wt, aAB, ou, eB, cj, eo, fl, k, y, hv); #c1(TNFRSF14) c2(NP_001284534) c3(tumor necrosis factor receptor superfamily member 14 isoform 2 precursor) c4( ) c5(111983, 111984, 111985, 111986, 111987, 111988, 111989, 111990, 111991, 111992, 111993, 111994) c6( ) c7(bM, T, dD, akc, fR, aw, ht, jh, fd, eq, bh, V, ai, aR, eW, xE, xl, aT, hu, qw, A, qZ, aW, eZ, fe, hp, iV, eE, ik, eo, y, it); #c1(TNFRSF19) c2(NP_001191388) c3(tumor necrosis factor receptor superfamily member 19 isoform 3) c4(81490) c5(81491, 81492, 81493, 81494, 81495) c6( ) c7(av, d, hk, Y, r, V, bz, q, de); #c1(TNFRSF19) c2(NP_061117) c3(tumor necrosis factor receptor superfamily member 19 isoform 1 precursor) c4(199193) c5(199194, 199195, 199196, 199197, 199198, 199199) c6( ) c7(av, d, hk, Y, r, V, bz, q, De); #c1(TNFRSF19) c2(NP_683760) c3(tumor necrosis factor receptor superfamily member 19 isoform 2 precursor) c4(242553) c5(242554, 242555, 242556, 242557, 242558, 242559, 242560, 242561, 242562, 242563, 242564) c6( ) c7(av, d, hk, Y, r, V, bz, q, De); #c1(TNFRSF1A) c2(NP_001056) c3(tumor necrosis factor receptor superfamily member 1A precursor) c4(34423, 34424) c5(34425, 34426, 34427, 34428, 34429, 34430, 34431, 34432, 34433, 34434, 34435, 34436, 34437, 34438, 34439, 34440) c6(34441, 34442, 34443, 34444) c7(bM, T, fR, ea, fd, cL, lR, aGX, mF, aBm, Tz, amW, yM, xE, aE, bW, dV, Ds, K, hu, hH, bc, bzH, aa, eG, aW, axu, asD, hl, mQ, ih, fc, vs, as, bw, uC, qr, mx, U, uo, k, m, R, bks, dD, sQ, My, aw, NJ, aj, ct, aBl, qW, cd, ai, eh, aPb, jz, bud, ju, Lv, lG, cU, Ne, dL, jy, h, NX, pi, nP, PT, mR, A, lb, fe, aq, CQ, bQL, cM, kT, RB, xO, Fi, hs, uz, ut, at, eo, fL, cOe, cOd, bD, B, bL, Dw, agx, fM, fG, Lx, jx, rf, aA, hz, v, j, OY, V, alx, J, cF, bjA, aWc, cdC, nX, aT, cy, UN, bq, an, af, eu, CB, Y, fO, aLJ, Ox, en, t, f, pb, iM, sn, c, ac, pZ, wO, aQd, AU, bxj, mf, EY, fD, bt, rA, ao, sY, aFe, qb, aD, iW, jh, bS, eq, pl, agz, wD, hX, dP, F, py, NY, oW, lQ, nJ, qE, uQ, d, je, ff, cr, eZ, aF, iV, Z, bz, dr, LB, Hw, gk, eU, axs, yE, Lj, aFb, aS, cP, Lz, ei, lf, ra, X, y); #c1(TNFRSF1B) c2(XP_011540365) c3(tumor necrosis factor receptor superfamily member 1B isoform X4) c4(338852, 338853) c5( ) c6( ) c7(bM, T, fR, e, al, bO, fd, bB, cL, ci, Tz, amW, aR, yM, aE, bW, dV, Ds, K, qM, aDZ, zy, H, hH, bzH, eG, LC, aW, Ls, mQ, hl, g, eE, cm, uC, bw, iT, mx, cj, uo, dW, wx, bBl, rt, dD, sQ, sw, My, ht, aw, aj, ct, aBl, uE, qW, cd, ai, eh, boz, ec, ju, Ne, h, ux, uV, bQx, Ha, A, mR, anP, CM, fe, CQ, jT, cM, Wt, RB, xO, Fi, at, bjK, ut, uz, fL, it, L, bL, Dw, jx, aee, aA, cz, v, j, OY, Qo, V, q, J, o, eA, cF, aWc, rS, aql, aT, cy, dz, UN, af, eu, bA, jf, kl, s, iU, Ue, Y, fO, r, en, t, pb, aff, bN, ck, sS, ac, c, aQd, hc, AU, mf, fD, bt, hS, sY, aFe, aZ, iW, jh, bS, eq, fu, pl, qi, hF, mC, wD, dP, py, oz, aCJ, oj, oW, hD, ji, lQ, aFl, qE, WZ, cH, ff, cr, eZ, dq, aF, iV, Z, qa, aYt, dR, LB, Bv, axs, yE, HP, bE, Lj, aFb, Hs, aS, ei, y, sH); #c1(TNFRSF21) c2(NP_055267) c3(tumor necrosis factor receptor superfamily member 21 precursor) c4(180168, 180169) c5(180170, 180171, 180172, 180173, 180174, 180175, 180176, 180177, 180178, 180179, 180180, 180181) c6( ) c7(dP, BN, c, U, hy, ww, uu, ey, ne, ad); #c1(TNFRSF6B) c2(NP_003814) c3(tumor necrosis factor receptor superfamily member 68 precursor) c4(141080, 141081) c5(141082, 141083, 141084, 141085, 141086, 141087, 141088, 141089, 141090, 141091, 141092, 141093, 141094, 141095, 141096) c6( ) c7(T, fM, hV, iW, ht, jh, aA, bB, n, aj, fd, fr, ci, q, J, ai, eA, eJ, BI, zw, aE, aT, cjN, ey, hu, pk, af, d, B, anP, je, aW, ff, cr, r, eZ, aq, fe, aF, en, g, as, t, jT, iV, iM, dB, bE, hc, Ny, m, y, it, X, On); #c1(TNFRSF8) c2(XP_011540743) c3(tumor necrosis factor receptor superfamily member 8 isoform X1) c4( ) c5(339317) c6( ) c7(bM, T, fM, dA, fR, eP, wu, iW, yG, al, eX, fd, jh, ctW, ci, hF, Tl, wa, x, fX, F, bIC, DK, TK, ju, apG, kH, aT, h, eV, je, ji, K, lQ, cOg, wm, pk, amY, Nr, asp, cOf, xR, aoX, vT, aW, Y, afF, A, Lh, eZ, po, afq, hp, bWt, zL, Ca, eE, bWs, uC, pb, Wt, asE, Nq, ck, Ta, BU, yR, iM, zr, iQ, iT, eU, eD, Zg, ajd, cj, zv, k, fD, i, y, KY, Tc); #c1(TNFRSF8) c2(XP_011540745) c3(tumor necrosis factor receptor superfamily member 8 isoform X3) [4( ) c5(339318) c6( ) c7(bM, T, fM, dA, fR, eP, wu, iW, yG, al, eX, fd, jh, ctW, ci, hF, Tl, wa, x, fX, F, bIC, DK, TK, ju, apG, kH, aT, h, eV, je, ji, K, IQ, cOg, wm, pk, amY, Nr, asp, cOf, xR, aoX, vT, aW, Y, afF, A, Lh, eZ, po, afq, hp, bWt, zL, Ca, eE, bWs, uC, pb, Wt, asE, Nq, ck, Ta, BU, yR, iM, zr, iQ, iT, eU, eD, Zg, ajd, cj, zv, k, fD, i, y, KY, Tc); #c1(TNFRSF8) c2(XP_011540746) c3(tumor necrosis factor receptor superfamily member 8 isoform X2) c4(339319) c5( ) c6( ) c7(bM, T, fM, dA, fR, eP, wu, iW, yG, al, eX, fd, jh, ctW, ci, hF, Tl, wa, x, fX, F, bIC, DK, TK, ju, apG, kH, aT, h, eV, je, ji, K, lQ, cOg, wm, pk, amY, Nr, asp, cOf, xR, aoX, vT, aW, Y, afF, A, Lh, eZ, po, afq, hp, bWt, zL, Ca, eE, bWs, uC, pb, Wt, asE, Nq, ck, Ta, BU, yR, iM, zr, iQ, iT, eU, eD, Zg, ajd, cj, zv, k, fD, i, y, KY, Tc); #c1(TNFSF10) c2(NP_001177871) c3(tumor necrosis factor ligand superfamily member 10 isoform 2) c4(73103) c5(73104, 73105, 73106, 73107, 73108) c6( ) c7(bM, T, os, aC, fR, pf, ea, if, e, fd, bB, axk, aoj, zh, ci, aAo, x, aE, Ou, K, hu, pk, eL, bc, cp, li, eG, Jl, aW, az, hl, g, as, eF, cm, bw, Qt, iP, eM, nB, iA, iT, mx, bru, cj, Zn, dW, k, m, iu, aiu, dD, ht, n, aj, fr, qN, zP, ai, br, eW, jz, no, zw, ec, kH, h, jy, Nf, dL, pi, dx, cY, A, cOh, mR, MD, PX, lb, hQ, ks, aq, Og, kp, pm, dB, Qu, hL, va, it, B, bL, fM, Ju, jx, aA, hy, bgR, V, q, J, eA, Yn, qK, yg, aT, aAn, Fx, adV, jj, bq, ae, af, ne, jf, qw, kl, s, nO, iU, oV, Y, ev, r, lo, aDm, en, cl, t, f, iK, pb, vk, ck, ov, bTk, kP, ac, iM, awN, AH, JH, im, bt, vi, hS, gV, Mb, kh, id, ow, em, oA, jh, oZ, dG, eq, jE, Fb, hF, ad, hX, dP, av, hd, ib, ds, oW, ji, d, BN, je, hG, ff, eH, Of, eZ, aF, iR, Bu, bz, es, eU, bE, apv, hE, kS, X, y); #c1(TNFSF10) c2(NP_003801) c3(tumor necrosis factor ligand superfamily member 10 isoform 1) c4(140993) c5(140994, 140995, 140996, 140997, 140998, 140999, 141000) c6( ) c7(bM, T, os, aC, fR, pf, ea, if, e, fd, bB, axk, aoj, zh, ci, aAo, x, aE, Ou, K, hu, pk, eL, bc, cp, li, eG, Jl, aW, az, hl, g, as, eF, cm, bw, Qt, iP, eM, nB, iA, iT, mx, bru, cj, Zn, dW, k, m, iu, aiu, dD, ht, n, aj, fr, qN, zP, ai, br, eW, jz, no, zw, ec, kH, h, jy, Nf, dL, pi, dx, cY, A, cOh, mR, MD, PX, lb, hQ, ks, aq, Og, kp, pm, dB, Qu, hL, va, it, B, bL, fM, Ju, jx, aA, hy, bgR, V, q, J, eA, Yn, qK, yg, aT, aAn, Fx, adV, jj, bq, ae, af, ne, jf, qw, kl, s, nO, iU, oV, Y, ev, r, lo, aDm, en, cl, t, f, iK, pb, vk, ck, ov, bTk, kP, ac, iM, awN, AH, JH, im, bt, vi, hS, gV, Mb, kh, id, ow, em, oA, jh, oZ, dG, eq, jE, Fb, hF, ad, hX, dP, av, hd, ib, ds, oW, ji, d, BN, je, hG, ff, eH, Of, eZ, aF, iR, Bu, bz, es, eU, bE, apv, hE, KS, X, y); #c1(TNFSF11) c2(NP_003692) c3(tumor necrosis factor ligand superfamily member 11 isoform 1) c4(140192) c5(140193, 140194, 140195, 140196, 140197, 140198, 140199, 140200) c6( ) c7(T, cjH, dD, ea, zs, oe, lH, e, EQ, aA, zt, aw, cL, aj, zh, ci, v, eq, wV, cNX, V, fr, ai, J, ad, bEa, cNT, amB, zw, xE, uT, pN, pX, yg, kH, aT, ae, bPQ, af, bA, aRR, azh, A, mR, je, aW, Y, bg, hl, eH, eZ, akB, cxe, cNU, jh, CQ, g, fd, pL, eF, iK, bz, aZz, dr, bQ, arv, eU, acW, iM, c, zr, yE, eM, cwA, go, aFb, cj, uz, bAD, azg, lS, zu, B, y, vQ); #c1(TNFSF11) c2(NP_143026) c3(tumor necrosis factor ligand superfamily member 11 isoform 2) c4(226604) c5(226605, 226606, 226607, 226608, 226609, 226610, 226611, 226612, 226613, 226614, 226615, 226616, 226617, 226618, 226619, 226620) c6( ) c7(T, cjH, dD, ea, zs, oe, lH, e, EQ, aA, zt, aw, cL, aj, zh, ci, v, eq, wV, cNX, V, fr, ai, J, ad, bEa, cNT, amB, zw, xE, uT, pN, pX, yg, kH, aT, ae, bPQ, af, bA, aRR, azh, A, mR, je, aW, Y, bg, hl, eH, eZ, akB, cxe, cNU, jh, CQ, g, fd, pL, eF, iK, bz, aZz, dr, bQ, arv, eU, acW, iM, c, zr, yE, eM, cwA, go, afb, cj, uz, bAD, azg, lS, zu, B, y, vQ); #c1(TNFSF12) c2(NP_003800) c3(tumor necrosis factor ligand superfamily member 12 proprotein) c4(140986) c5(140987, 140988, 140989, 140990, 140991, 140992) c6( ) c7(bL, dD, ea, aA, bB, v, eq, pz, eA, ad, hb, M, aT, ae, d, qt, aW, eZ, aF, D, bE, y); #c1(TNFSF12-TNFSF13) c2(NP_742086) c3(TNFSF12-TNFSF13 protein) c4(248054) c5(248055, 248056, 248057, 248058, 248059, 248060, 248061, 248062, 248063, 248064, 248065, 248066) c6(248067, 248068) c7(bL, dD, d, hb, aW, ea, bE, qt, M, aT, aA, bB, eZ, v, eq, af, y, ae, eA, ad); #c1(TNFSF13) c2(NP_001185552) c3(tumor necrosis factor ligand superfamily member 13 isoform zeta) c4(75832) c5(75833, 75834, 75835, 75836, 75837, 75838, 75839, 75840, 75841) c6(75842, 75843) c7(Ob, bM, T, dD, fM, dA, ea, iW, aw, eX, fN, aA, fd, jh, j, ci, eq, V, q, eA, Wu, aT, cU, h, eV, hn, ey, ji, d, s, cW, oV, ff, eZ, r, Io, aF, hp, en, g, eE, ve, bz, pb, Wt, ck, zr, bE, cj, eo, fL, lf, bt, Ze, y); #c1(TNFSF13) c2(NP_001185553) c3(tumor necrosis factor ligand superfamily member 13 isoform eta) c4(75844) c5(75845, 75846, 75847, 75848, 75849, 75850, 75851) c6(75852, 75853) c7(Ob, bM, T, dD, fM, dA, ea, iW, aw, eX, fN, aA, fd, jh, j, ci, eq, V, q, eA, Wu, aT, cU, h, eV, hn, ey, ji, d, s, cW, oV, ff, eZ, r, lo, aF, hp, en, g, eE, ve, bz, pb, Wt, ck, zr, bE, cj, eo, fL, lf, bt, Ze, y); #c1(TNFSF13) c2(NP_003799) c3(tumor necrosis factor ligand superfamily member 13 isoform alpha proprotein) c4(140975) c5(140976, 140977, 140978, 140979, 140980, 140981, 140982, 140983) c6(140984, 140985) c7(Ob, bM, T, dD, fM, dA, ea, iW, aw, eX, fN, aA, fd, jh, j, ci, eq, V, q, eA, Wu, aT, cU, h, eV, hn, ey, ji, d, s, cW, oV, ff, eZ, r, lo, aF, hp, en, g, eE, ve, bz, pb, Wt, ck, zr, bE, cj, eo, fL, lf, bt, Ze, y); #c1(TNFSF13) c2(NP_742084) c3(tumor necrosis factor ligand superfamily member 13 isoform beta proprotein) c4(248032) c5(248033, 248034, 248035, 248036, 248037, 248038, 248039, 248040) c6(248041, 248042) c7(Ob, bM, T, dD, fM, dA, ea, iW, aw, eX, fN, aA, fd, jh, j, ci, eq, V, q, eA, Wu, aT, cU, h, eV, hn, ey, ji, d, s, cW, oV, ff, eZ, r, lo, aF, hp, en, g, eE, ve, bz, pb, Wt, ck, zr, bE, cj, eo, fL, lf, bt, Ze, y); #c1(TNFSF13) c2(NP_742085) c3(tumor necrosis factor ligand superfamily member 13 isoform gamma proprotein) c4(248043) c5(248044, 248045, 248046, 248047, 248048, 248049, 248050, 248051) c6(248052, 248053) c7(Ob, bM, T, dD, fM, dA, ea, iW, aw, eX, fN, aA, fd, jh, j, ci, eq, V, q, eA, Wu, aT, cU, h, eV, hn, ey, ji, d, s, cW, oV, ff, eZ, r, lo, aF, hp, en, g, eE, ve, bz, pb, Wt, ck, zr, bE, cj, eo, fL, lf, bt, Ze, y); #c1(TNFSF13B) c2(NP_001139117) c3(tumor necrosis factor ligand superfamily member 13B isoform 2) c4(58705) c5(58706, 58707, 58708, 58709, 58710, 58711) c6( ) c7(bM, T, fR, fd, bh, x, bv, xE, aE, dV, zy, hH, pk, DY, axu, eF, ve, uC, cj, bAD, k, cOj, cZ, aFQ, fN, apt, uE, cd, ai, ec, aow, aMj, cU, h, eV, ey, dd, Og, kp, cOi, kk, Wt, eo, fL, fM, dA, jx, eX, v, j, V, eA, acH, jj, ae, hM, oV, Io, en, pb, OC, ck, c, yW, yH, ij, iW, jh, cOk, mC, ad, aoH, dP, bTr, lQ, Od, aOh, uQ, ff, aFE, eH, eZ, Of, BG, iV, zr, eB, lf, kS, y, acG); #c1(TNFSF13B) c2(NP_006564) c3(tumor necrosis factor ligand superfamily member 13B isoform 1) c4(166265, 166266) c5(166267, 166268, 166269, 166270, 166271, 166272) c6( ) c7(bM, T, FR, fd, bh, x, bv, xE, aE, dV, zy, hH, pk, DY, axu, eF, ve, uC, cj, bAD, k, cOj, cZ, aFQ, fN, apt, uE, cd, ai, ec, aow, aMj, cU, h, eV, ey, dd, Og, kp, cOi, kk, Wt, eo, fL, fM, dA, jx, eX, v, j, V, eA, acH, jj, ae, hM, oV, lo, en, pb, OC, ck, c, yW, yH, ij, iW, jh, cOk, mC, ad, aoH, dP, bTr, lQ, Od, aOh, uQ, ff, aFE, eH, eZ, Of, BG, iV, zr, eB, lf, KS, y, acG); #c1(TNFSF13B) c2(XP_005254086) c3(tumor necrosis factor ligand superfamily member 13B isoform X1) c4( ) c5(272876, 272877, 272878, 272879, 272880, 272881) c6( ) c7(bM, T, FR, fd, bh, x, bv, xE, aE, dV, zy, hH, pk, DY, axu, eF, ve, uC, cj, bAD, k, cOj, cZ, aFQ, fN, apt, uE, cd, ai, ec, aow, aMj, cU, h, eV, ey, dd, Og, kp, cli, kk, Wt, eo, fL, fM, dA, jx, eX, v, j, V, eA, acH, jj, ae, hM, oV, lo, en, pb, OC, ck, c, yW, yH, ij, iW, jh, cOk, mC, ad, aoH, dP, bTr, lQ, Od, aOh, uQ, ff, aFE, eH, eZ, Of, BG, iV, zr, eB, lf, kS, y, acG); #c1(TNFSF14) c2(NP_003798) c3(tumor necrosis factor ligand superfamily member 14 isoform 1 precursor) c4(140964) c5(140965, 140966, 140967, 140968, 140969, 140970, 140971, 140972, 140973, 140974) c6( ) c7(bL, bM, T, fM, qb, fG, if, jh, aA, fd, rf, aj, v, lN, ci, qN, V, q, J, ai, eW, cF, xE, rS, M, h, ji, bU, Nr, iZ, cH, aW, Y, vj, Du, r, fe, po, aF, eE, wA, Nq, y, ac, c, lI, cj, lC, B, EB, vi); #c1(TNFSF14) c2(NP_742011) c3(tumor necrosis factor ligand superfamily member 14 isoform 2) c4(247590) c5(247591, 247592, 247593, 247594, 247595, 247596) c6( ) c7(bL, bM, T, fM, qb, fG, if, jh, aA, fd, rf, aj, v, lN, ci, qN, V, q, J, ai, eW, cF, xE, rS, M, h, ji, bU, Nr, iZ, cH, aW, Y, vj, Du, r, fe, po, af, eE, wA, Nq, y, ac, c, lI, cj, lC, B, EB, vi); #c1(TNFSF15) c2(NP_001191273) c3(tumor necrosis factor ligand superfamily member 15 isoform VEGI-192) c4( ) c5(81265, 81266, 81267, 81268, 81269, 81270) c6( ) c7(T, dD, eq, ai, AW, xE, pe, bc, aGP, A, Di, cr, ge, dN, fe, iV, pL, t, acD, acC, gt, Ji, m, cOl, y, ra); #c1(TNFSF15) c2(NP_005109) c3 (tumor necrosis factor ligand superfamily member 15 isoform VEGI-251 precursor) c4(152964, 152965) c5(152966, 152967, 152968, 152969, 152970, 152971) c6( ) c7(T, dD, eq, ai, AW, xE, pe, bc, aGP, A, Di, cr, ge, dN, fe, iV, pL, t, acD, acC, gt, Ji, m, col, y, ra); #c1(TNFSF18) c2(NP_005083) c3(tumor necrosis factor ligand superfamily member 18) c4(152737, 152738) c5(152739, 152740, 152741, 152742, 152743, 152744, 152745, 152746, 152747, 152748, 152749, 152750) c6( ) c7(ck, jx, cj, aT, fd, fe, hp, eE, bt); #c1(TNFSF4) c2(NP_001284491) c3 (tumor necrosis factor ligand superfamily member 4 isoform 2) c4(111912) c5(111913, 111914, 111915, 111916, 111917, 111918, 111919, 111920, 111921, 111922) c6( ) c7(bM, T, dD, eR, ea, e, jx, aw, ex, cz, fd, aj, eq, eW, hX, cF, dY, ec, ds, M, h, eV, ji, bay, bc, aEV, qw, A, je, eZ, fe, yq, hp, pb, lx, eK, ck, iM, ac, cOm, bt, X); #c1(TNFSF4) c2(NP_003317) c3(tumor necrosis factor ligand superfamily member 4 isoform 1) c4( ) c5(137452, 137453, 137454, 137455, 137456) c6( ) c7(bM, T, dD, eR, ea, e, jx, aw, eX, cz, fd, aj, eq, eW, hX, cF, dY, ec, ds, M, h, eV, ji, bay, bc, aEV, qw, A, je, eZ, fe, yq, hp, pb, lx, eK, ck, iM, ac, cOm, bt, X); #c1(TNFSF4) c2(XP_011508266) c3(tumor necrosis factor ligand superfamily member 4 isoform X1) c4(298963) c5(298964) c6( ) c7(bM, T, dD, eR, ea, e, jx, aw, eX, cz, fd, aj, eq, eW, hX, cF, dY, ec, ds, M, h, eV, ji, bay, bc, aEV, qw, A, je, eZ, fe, yq, hp, pb, lx, eK, ck, iM, ac, cOm, bt, X); #c1(TNFSF8) c2(NP_001235) c3 (tumor necrosis factor ligand superfamily member 8 isoform 1) c4(87879) c5(87880, 87881, 87882, 87883, 87884, 87885) c6( ) c7(T, iW, oe, ex, jh, fd, ci, bX, zw, bIC, eV, ji, lQ, bay, Us, aEV, A, asp, vT, cr, eE, bz, ck, rR, Dy, eD, cj, bt); #c1(TNFSF9) c2(NP_003802) c3(tumor necrosis factor ligand superfamily member 9) c4(141001) c5(141002, 141003, 141004, 141005, 141006, 141007) c6( ) c7(T, dD, fM, ea, aA, cz, fd, eq, V, ad, vV, Rq, ds, h, bay, ae, bc, aEV, g, jT, t, cm, ck, D, ac, cj, y); #c1(TNIK) c2(NP_001155032) c3(TRAF2 and NCK-interacting protein kinase isoform 2) c4(61273) c5( ) c6( ) c7(T, aW, r, aT, j, lQ, Pk, q); #c1(TNIK) c2(NP_001155033) c3(TRAF2 and NCK-interacting protein kinase isoform 3) c4(61274, 61275) c5( ) c6( ) c7(T, aW, r, aT, j, lQ, Pk, q); #c1(TNIK) c2(NP_001155034) c3(TRAF2 and NCK-interacting protein kinase isoform 4) c4(61276) c5(61277, 61278, 61279, 61280, 61281, 61282, 61283, 61284, 61285, 61286, 61287, 61288) c6( ) c7(T, aW, r, aT, j, lQ, Pk, q); #c1(TNIK) c2(NP_001155035) c3(TRAF2 and NCK-interacting protein kinase isoform 5) c4(61289, 61290) c5( ) c6( ) c7(T, aW, r, aT, j, lQ, Pk, q); #c1(TNIK) c2(NP_001155036) c3(TRAF2 and NCK-interacting protein kinase isoform 6) c4(61291) c5(61292, 61293, 61294, 61295, 61296, 61297, 61298, 61299, 61300, 61301, 61302, 61303) c6( ) c7(T, aW, r, aT, j, lQ, Pk, q); #c1(TNIK) c2(NP_001155037) c3(TRAF2 and NCK-interacting protein kinase isoform 7) c4(61304) c5(61305, 61306, 61307, 61308, 61309, 61310, 61311, 61312, 61313, 61314, 61315, 61316, 61317, 61318, 61319, 61320, 61321, 61322) c6( ) c7(T, aW, r, aT, j, lQ, Pk, q); #c1(TNIK) c2(NP_001155038) c3(TRAF2 and NCK-interacting protein kinase isoform 8) c4(61323) c5( ) c6( ) c7(T, aW, r, aT, j, lQ, Pk, q); #c1(TNIK) c2(NP_055843) c3(TRAF2 and NCK-interacting protein kinase isoform 1) c4(183856) c5(183857, 183858, 183859, 183860, 183861, 183862, 183863, 183864, 183865, 183866, 183867, 183868, 183869, 183870, 183871, 183872, 183873, 183874) c6( ) c7(T, aW, r, aT, j, lQ, Pk, q); #c1(TNIPI) c2(NP_001239315) c3(TNFAIP3-interacting protein 1 isoform 2) c4( ) c5(88412) c6( ) c7(sb, bM, hM, mR, aWF, Jr, Ue, fN, aT, at, cr, eZ, dL, C, y, pb, wD); #c1(TNIPI) c2(NP_001239322) c3(TNFAIP3-interacting protein 1 isoform 4) c4(88413) c5(88414, 88415, 88416, 88417, 88418, 88419, 88420, 88421, 88422, 88423) c6( ) c7(sb, bM, hM, mR, aWF, Jr, Ue, fN, aT, at, cr, eZ, dL, C, y, pb, wD); #c1(TNIPI) c2(NP_001245385) c3(TNFAIP3-interacting protein 1 isoform 6) c4(93372, 93373, 93374, 93375, 93376, 93377, 93378) c5(93379, 93380, 93381, 93382, 93383, 93384, 93385, 93386, 93387, 93388, 93389, 93390, 93391, 93392, 93393, 93394, 93395, 93396, 93397, 93398, 93399, 93400, 93401, 93402, 93403, 93404, 93405, 93406, 93407, 93408, 93409, 93410, 93411, 93412, 93413, 93414, 93415, 93416, 93417, 93418, 93419, 93420) c6( ) c7(sb, bM, hM, mR, aWF, Jr, Ue, fN, aT, at, cr, eZ, dL, C, y, pb, wD); #c1(TNIPI) c2(XP_006714814) c3(TNFAIP3-interacting protein 1 isoform X3) c4(288234, 288235) c5(288236, 288237, 288238, 288239, 288240, 288241, 288242, 288243, 288244, 288245, 288246, 288247) c6( ) c7(sb, bM, hM, mR, aWF, Jr, Ue, fN, al, at, cr, eZ, dL, C, y, pb, wD); #c1(TNIPI) c2(XP_011535840) c3(TNFAIP3-interacting protein 1 isoform X2) c4(333104, 333105, 333106) c5(333107, 333108, 333109, 333110, 333111, 333112, 333113, 333114, 333115, 333116, 333117, 333118, 333119, 333120, 333121, 333122, 333123, 333124, 333125, 333126, 333127, 333128, 333129, 333130, 333131, 333132, 333133, 333134, 333135, 333136) c6( ) c7(sb, bM, hM, mR, aWF, Jr, Ue, fN, aT, at, cr, eZ, dL, C, y, pb, wD); #c1(TNIP2) c2(NP_001154999) c3(TNFAIP3-interacting protein 2 isoform 2) c4( ) c5(61255, 61256, 61257, 61258, 61259, 61260) c6( ) c7(ck, y); #c1(TNIP2) c2(NP_077285) c3(TNFAIP3-interacting protein 2 isoform 1) c4(214934, 214935) c5(214936, 214937, 214938, 214939, 214940, 214941) c6( ) c7(ck, y); #c1(TNIP3) c2(NP_001122315) c3(TNFAIP3-interacting protein 3 isoform 2) c4(46510) c5(46511, 46512, 46513, 46514, 46515) c6( ) c7(azU, fq, C); #c1(TNIP3) c2(NP_001231693) c3(TNFAIP3-interacting protein 3 isoform 3) c4( ) c5(87439) c6( ) c7(azU, fq, C); #c1(TNIP3) c2(NP_079149) c3(TNFAIP3-interacting protein 3 isoform 1) c4(217264) c5(217265, 217266, 217267, 217268, 217269, 217270, 217271, 217272, 217273, 217274) c6( ) c7(azU, fq, C); #c1(TNIP3) c2(XP_011530558) c3(TNFAIP3-interacting protein 3 isoform X1) c4( ) c5(326483, 326484) c6( ) c7(azU, fq, C); #c1(TNK1) c2(XP_011522347) c3(non-receptor tyrosine-protein kinase TNK1 isoform X1) c4(317019) c5(317020, 317021, 317022, 317023, 317024, 317025, 317026, 317027, 317028, 317029) c6( ) c7(ck, af, aE); #c1(TNK2) c2(NP_001010938) c3(activated CDC42 kinase 1 isoform 2) c4(24253, 24254) c5(24255, 24256, 24257, 24258, 24259, 24260, 24261, 24262, 24263, 24264, 24265, 24266) c6( ) c7(gh, T, es, eM, iA, aW, Ls, ha, aT, aA, aq, By, aF, fr, g, ae, iu, ad); #c1(TNK2) c2(NP_005772) c3(activated CDC42 kinase 1 isoform 1) c4(159225) c5(159226, 159227, 159228, 159229, 159230, 159231, 159232, 159233, 159234) c6( ) c7(gh, T, es, eM, iA, aW, Ls, ha, aT, aA, aq, By, aF, fr, g, ae, iu, ad); #c1(TNK2) c2(XP_005269325) c3 (activated CDC42 kinase 1 isoform X4) c4( ) c5(282554) c6( ) c7(gh, T, es, eM, iA, aW, Ls, ha, al, aA, aq, By, aF, fr, g, ae, iu, ad); #c1(TNK2) c2(XP_005269332) c3(activated CDC42 kinase 1 isoform X9) c4( ) c5(282555) c6( ) c7(gh, T, es, eM, iA, aW, Ls, ha, aT, aA, aq, By, aF, fr, g, ae, iu, ad); #c1(TNK2) c2(XP_011510619) c3(activated CDC42 kinase 1 isoform X1) c4( ) c5(301076, 301077, 301078, 301079, 301080, 301081) c6( ) c7(gh, T, es, eM, iA, aW, Ls, ha, aT, aA, aq, By, aF, fr g, ae, iu, ad); #c1(TNKS) c2(NP_003738) c3(tankyrase-1) c4(140538) c5(140539, 140540, 140541, 140542, 140543, 140544) c6( ) c7(bM, T, id, e, aA, fd, n, v, ci, eA, eW, M, aT, dL, eL, NM, aW, af, as, cm, bz, ac, at, aNL); #c1(TNKS2) c2(NP_079511) c3(tankyrase-2) c4(218446) c5(218447, 218448, 218449, 218450, 218451, 218452) c6( ) c7(sS, ac, aW, yk, e, if, ff, hL, aT, n, en, cL, qN, bz); #c1(TNMD) c2(NP_071427) c3(tenomodulin) c4(211608) c5(211609, 211610, 211611, 211612, 211613, 211614) c6( ) c7(bL, bF, W, ac, eM, LC, baS, uo, dq, aj, mf, v, eA, ei, aFf, af, J); #c1(TNN) c2(NP_071376) c3(tenascin-N precursor) c4(211251) c5(211252, 211253, 211254, 211255, 211256, 211257, 211258, 211259) c6(211260, 211261) c7(aT); #c1(TNN) c2(XP_011508171) c3(tenascin-N isoform X3) c4(298721) c5(298722, 298723, 298724, 298725, 298726, 298727, 298728, 298729) c6(298730, 298731) c7(aT); #c1(TNNC1) c2(NP_003271) c3(troponin C, slow skeletal and cardiac muscles) c4(136963) c5(136964, 136965, 136966, 136967, 136968, 136969) c6( ) c7(bM, T, AY, fG, n, cc, aE, cOo, aT, vc, K, zp, cOn, anR, d, aW, ff, kp, en, mo, aYo, g, pb, eM, mx, ro, awa); #c1(TNNI1) c2(NP_003272) c3(troponin I, slow skeletal muscle) c4(136970, 136971, 136972) c5(136973, 136974, 136975, 136976, 136977, 136978, 136979, 136980, 136981, 136982, 136983, 136984, 136985, 136986, 136987, 136988, 136989, 136990, 136991, 136992, 136993, 136994, 136995, 136996, 136997, 136998, 136999, 137000, 137001, 137002) c6( ) c7(bM, eN, lS); #c1(TNNI2) c2(NP_001139313) c3(troponin I, fast skeletal muscle isoform 2) c4(59149) c5(59150, 59151, 59152, 59153, 59154, 59155) c6( ) c7(chE, oj); #c1(TNNI2) c2(NP_003273) c3(troponin I, fast skeletal muscle isoform 1) c4(137003) c5(137004, 137005, 137006, 137007, 137008, 137009, 137010, 137011, 137012, 137013, 137014, 137015, 137016, 137017) c6( ) c7(chE, oj); #c1(TNNI3) c2(NP_000354) c3(troponin 1, cardiac muscle) c4(7825) c5(7826, 7827, 7828, 7829, 7830, 7831) c6( ) c7(eR, Hm, aw, akA, bS, ct, cd, cOq, cc, rS, cOs, oj, cy, vc, cOp, af, hm, AX, anR, uY, g, mo, Z, aYo, YF, akF, cL, ac, cOr, mx, gZ, wx, gq, sH, vQ); #c1(TNNT1) c2(NP_001278703) c3(troponin T, slow skeletal muscle isoform c) c4(109977, 109978) c5(109979, 109980, 109981, 109982, 109983, 109984, 109985, 109986, 109987, 109988, 109989, 109990, 109991, 109992, 109993, 109994, 109995, 109996, 109997, 109998, 109999, 110000, 110001, 110002) c6( ) c7(cc, Am, cOt, dR); #c1(TNNT1) c2(NP_003274) c3(troponin T, slow skeletal muscle isoform a) c4(137018) c5(137019, 137020, 137021, 137022, 137023, 137024, 137025, 137026, 137027, 137028) c6( ) c7(cc, Am, cOt, dR); #c1(TNNT1) c2(XP_006723406) c3(troponin T, slow skeletal muscle isoform X4) c4(295198) c5( ) c6( ) c7(cc, Am, cOt, dR); #c1(TNNT1) c2(XP_011525548) c3(troponin T, slow skeletal muscle isoform X5) c4(319976) c5( ) c6( ) c7(cc, Am, cot, dR); #c1(TNNT2) c2(NP_001001430) c3(troponin T, cardiac muscle isoform 2) c4(18397, 18398, 18399, 18400) c5( ) c6( ) c7(dD, cun, e, cOu, aw, cyE, j, eq, cd, cc, cOs, cOw, vc, cOv, anR, mo, aYo, dr, bfW, wT, cl, ac, mx, lI, gZ, at, gq, jr, vQ); #c1(TNNT2) c2(NP_001001431) c3(troponin T, cardiac muscle isoform 3) c4(18401, 18402, 18403) c5(18404, 18405, 18406, 18407, 18408, 18409, 18410, 18411, 18412, 18413, 18414, 18415, 18416, 18417, 18418, 18419, 18420, 18421, 18422, 18423, 18424, 18425) c6( ) c7(dD, cun, e, cOu, aw, cyE, j, eq, cd, cc, cOs, cw, vc, cOv, anR, mo, aYo, dr, bfW, wT, cl, ac, mx, lI, gZ, at, gq, jr, vQ); #c1(TNNT2) c2(XP_011508242) c3(troponin T, cardiac muscle isoform X4) c4( ) c5(298883) c6( ) c7(dD, cun, e, cOu, aw, cyE, j, eq, cd, cc, cos, cOw, vc, cOv, anR, mo, aYo, dr, bfW, wT, cl, ac, mx, lI, gZ, at, gq, jr, vQ); #c1(TNNT2) c2(XP_011508245) c3(troponin T, cardiac muscle isoform X7) c4(298884) c5( ) c6( ) c7(dD, cun, e, cOu, aw, cyE, j, eq, cd, cc, cOs, cOw, vc, cOv, anR, mo, aYo, dr, bfW, wT, cl, ac, mx, lI, gZ, at, gq, jr, vQ); #c1(TNNT2) c2(XP_011508246) c3(troponin T, cardiac muscle isoform X8) c4( ) c5(298885, 298886, 298887, 298888, 298889, 298890, 298891, 298892, 298893, 298894, 298895, 298896, 298897, 298898, 298899, 298900, 298901, 298902, 298903, 298904) c6( ) c7(dD, cun, e, cOu, aw, cyE, j, eq, cd, cc, cOs, cOw, vc, cv, anR, mo, aYo, dr, bfW, wT, cl, ac, mx, lI, gZ, at, gq, jr, vQ); #c1(TNNT2) c2(XP_011508248) c3(troponin T, cardiac muscle isoform X10) c4( ) c5(298905, 298906, 298907, 298908, 298909, 298910, 298911, 298912, 298913, 298914, 298915, 298916, 298917, 298918, 298919, 298920, 298921) c6( ) c7(dD, cun, e, cOu, aw, cyE, j, eq, cd, cc, cOs, cOw, vc, cOv, anR, mo, aYo, dr, bfW, wT, cl, ac, mx, II, gZ, at, gq, jr, vQ); #c1(TNNT3) c2(NP_001036245) c3(troponin T, fast skeletal muscle isoform 3) c4(34092) c5( ) c6( ) c7(chE, XW, chF); #c1(TNNT3) c2(NP_001036246) c3(troponin T, fast skeletal muscle isoform 2) c4(34093) c5( ) c6( ) c7(chE, XW, chF); #c1(TNNT3) c2(NP_001284575) c3(troponin T, fast skeletal muscle isoform 4) c4(112069) c5( ) c6( ) c7(chE, XW, chF); #c1(TNNT3) c2(NP_006748) c3(troponin T, fast skeletal muscle isoform 1) c4(167668) c5( ) c6( ) c7(chE, XW, chF); #c1(TNNT3) c2(XP_006718351) c3(troponin T, fast skeletal muscle isoform X15) c4(291712, 291713) c5( ) c6( ) c7(chE, XW, chF); #c1(TNNT3) c2(XP_006718355) c3(troponin T, fast skeletal muscle isoform X4) c4(291714) c5( ) c6( ) c7(chE, XW, chF); #c1(TNNT3) c2(XP_006718356) c3(troponin T, fast skeletal muscle isoform X5) c4(291715) c5( ) c6( ) c7(chE, XW, chF); #c1(TNNT3) c2(XP_006718358) c3(troponin T, fast skeletal muscle isoform X7) c4(291716) c5( ) c6( ) c7(chE, XW, chF); #c1(TNNT3) c2(XP_006718363) c3(troponin T, fast skeletal muscle isoform X12) c4(291717) c5( ) c6( ) c7(chE, XW, chF); #c1(TNNT3) c2(XP_006718364) c3(troponin T, fast skeletal muscle isoform X13) c4(29/718) c5(291719, 291720, 291721, 29/722, 29/723, 29/724, 29/725, 291726, 291727, 291728, 29/729, 291730) c6( ) c7(chE, XW, chF); #c1(TNNT3) c2(XP_006718365) c3(troponin T, fast skeletal muscle isoform X14) c4( ) c5(291731, 291732, 291733, 291734, 291735, 291736, 291737, 291738, 291739, 291740, 291741, 291742, 291743, 291744, 291745, 291746, 291747, 291748, 291749, 291750, 291751, 291752, 291753, 291754, 291755, 291756, 291757, 291758, 291759, 291760, 29/761, 29/762, 291763, 291764, 29/765, 291766, 291767, 291768, 291769, 291770, 291771, 291772, 291773, 291774, 29/775, 291776, 291777, 291778, 291779, 291780, 291781, 291782, 291783, 291784, 291785, 291786, 291787, 291788, 291789, 291790) c6( ) c7(chE, XW, chF); #c1(TNNT3) c2(XP_011518645) c3(troponin T, fast skeletal muscle isoform X1) c4(312981) c5( ) c6( ) c7(chE, XW, chF); #c1(TNP1) c2(NP_003275) c3(spermatid nuclear transition protein 1) c4(137029) c5(137030, 137031, 137032, 137033, 137034, 137035) c6( ) c7(T, gU, GY, ND, aW, aT, vH, y, ai); #c1(TNP2) c2(NP_005416) c3(nuclear transition protein 2) c4(155867) c5(155868, 155869, 155870, 155871, 155872, 155873) c6( ) c7(vH, A, gU, ND); #c1(TNP01) c2(NP_002261) c3 (transportin-1 isoform 1) c4(127036, 127037) c5(127038, 127039, 127040, 127041, 127042, 127043, 127044, 127045, 127046, 127047, 127048, 127049, 127050, 127051, 127052) c6( ) c7(bM, T, Dw, dA, aD, fG, lH, bLS, jh, fd, aj, j, ci, UX, fr, pz, q, J, cd, aXI, aoH, eW, zw, aXC, xE, aWc, M, kH, cU, aT, dV, h, K, mJ, azh, ceo, je, caK, bf, aW, xz, r, fe, aq, lo, iV, Bu, kC, pb, QA, aBs, LB, ck, pa, iM, aDA, c, bE, wO, hc, aS, fD, bwg, y, bt, B); #c1(TNP01) c2(XP_005248557) c3(transportin-1 isoform X1) c4(268459) c5(268460, 268461, 268462, 268463, 268464) c6( ) c7(bM, T, Dw, dA, aD, fG, lH, bLS, jh, fd, aj, j, ci, UX, fr, pz, q, J, cd, aXI, aoH, eW, zw, aXC, xE, aWc, M, kH, cU, aT, dV, h, K, mJ, azh, ceo, je, caK, bf, aW, xz, r, fe, aq, Io, iV, Bu, kC, pb, QA, aBs, LB, ck, pa, iM, aDA, c, bE, wO, hc, aS, fD, bwg, y, bt, B); #c1(TNP01) c2(XP_005248558) c3(transportin-1 isoform X2) c4(268465) c5(268466, 268467, 268468, 268469, 268470, 268471, 268472) c6( ) c7(bM, T, Dw, dA, aD, fG, lH, bLS, jh, fd, aj, j, ci, UX, fr, pz, q, J, cd, aXI, aoH, eW, zw, aXC, xE, aWc, M, kH, cU, aT, dV, h, K, mJ, azh, ceo, je, caK, bf, aW, xz, r, fe, aq, lo, iV, Bu, kC, pb, QA, aBs, LB, ck, pa, iM, aDA, c, bE, wO, hc, aS, fD, bwg, y, bt, B); #c1(TNP02) c2(NP_001129667) c3(transportin-2 isoform 2) c4(52126, 52127, 52128) c5(52129, 52130, 52131, 52132, 52133, 52134, 52135, 52136, 52137, 52138, 52139, 52140, 52141, 52142, 52143, 52144, 52145, 52146, 52147, 52148, 52149, 52150, 52151, 52152, 52153, 52154, 52155, 52156) c6( ) c7(kl, an); #c1(TNP02) c2(NP_001129668) c3(transportin-2 isoform 1) c4(52157, 52158) c5(52159, 52160, 52161, 52162, 52163, 52164, 52165, 52166, 52167, 52168) c6( ) c7(kl, an); #c1(TNP03) c2(NP_001177957) c3 (transportin-3 isoform 2) c4(73236) c5(73237, 73238, 73239, 73240, 73241, 73242) c6( ) c7(km, oj, h, eZ, lo, gn, pb, y, cOx, bc); #c1(TNP03) c2(NP_036602) c3(transportin-3 isoform 1) c4(174737, 174738) c5(174739, 174740, 174741, 174742, 174743, 174744, 174745, 174746, 174747, 174748, 174749, 174750, 174751, 174752, 174753, 174754) c6( ) c7(km, oj, h, eZ, lo, gn, pb, y, cOx, bc); #c1(TNR) c2(XP_011508249) c3(tenascin-R isoform X1) c4(298922) c5(298923, 298924, 298925, 298926, 298927, 298928, 298929, 298930, 298931, 298932) c6( ) c7(jf, sY, T, d, fm, oe, U, aT, bK, ey, nA, Hp, uC, Lg, Fc); #c1(TNR) c2(XP_011508250) c3(tenascin-R isoform X2) c4(298933) c5(298934, 298935, 298936, 298937, 298938, 298939) c6( ) c7(jf, sY, T, d, fm, oe, U, aT, bK, ey, nA, Hp, uC, Lg, Fc); #c1(TNR) c2(XP_011508251) c3(tenascin-R isoform X3) c4(298940, 298941) c5(298942, 298943, 298944, 298945, 298946, 298947) c6( ) c7(jf, sY, T, d, fm, oe, U, aT, bK, ey, nA, Hp, uC, Lg, Fc); #c1(TNRC18) c2(NP_001073964) c3(trinucleotide repeat-containing gene 18 protein) c4(36717, 36718, 36719, 36720) c5(36721, 36722, 36723, 36724, 36725, 36726, 36727, 36728, 36729, 36730, 36731, 36732, 36733) c6( ) c7(aw); #c1(TNRC18) c2(XP_011513890) c3(trinucleotide repeat-containing gene 18 protein isoform X1) c4(306316) c5(306317, 306318, 306319, 306320, 306321, 306322, 306323, 306324, 306325, 306326, 306327, 306328, 306329, 306330, 306331, 306332, 306333, 306334) c6( ) c7(aw); #c1(TNRC6A) c2(NP_055309) c3(trinucleotide repeat-containing gene 6A protein) c4(180388) c5(180389, 180390, 180391, 180392, 180393, 180394) c6( ) c7(T, aA, r); #c1(TNRC6A) c2(XP_005255314) c3(trinucleotide repeat-containing gene 6A protein isoform X7) c4( ) c5(273607, 273608, 273609, 273610, 273611, 273612) c6( ) c7(T, aA, r); #c1(TNRC6A) c2(XP_011544094) c3(trinucleotide repeat-containing gene 6A protein isoform X2) c4(343266) c5( ) c6( ) c7(T, aA, r); #c1(TNRC6B) c2(NP_001020014) c3(trinucleotide repeat-containing gene 68 protein isoform 3) c4(28019, 28020) c5(28021, 28022, 28023, 28024, 28025, 28026, 28027, 28028, 28029, 28030, 28031, 28032, 28033, 28034, 28035, 28036) c6( ) c7(op, kl, ad, vA); #c1(TNRC6B) c2(NP_001155973) c3(trinucleotide repeat-containing gene 68 protein isoform 1) c4(61673) c5(61674, 61675, 61676, 61677, 61678, 61679) c6( ) c7(op, kl, ad, vA); #c1(TNRC6B) c2(NP_055903) c3(trinucleotide repeat-containing gene 68 protein isoform 2) c4(184359, 184360) c5(184361, 184362, 184363, 184364, 184365, 184366, 184367, 184368, 184369, 184370, 184371, 184372, 184373) c6( ) c7(op, kl, ad, vA); #c1(TNS1) c2(NP_072174) c3(tensin-1) c4(212777) c5(212778, 212779, 212780, 212781, 212782, 212783) c6( ) c7(T, aA, cd, q, mC, ad, eA, Na, aT, bj, K, ae, d, aW, Y, eZ, r, aq, af, iR, ck, es, mO, qJ, c, eM, TJ, iA, at, Dp, m, fL, B, hS); #c1(TNS1) c2(XP_011510017) c3(tensin-1 isoform X5) c4(300638) c5( ) c6( ) c7(T, aA, cd, q, mC, ad, eA, Na, aT, bj, K, ae, d, aW, Y, eZ, r, aq, aF, iR, ck, es, mO, qJ, c, eM, TJ, iA, at, Dp, m, fL, B, hS); #c1(TNS1) c2(XP_011510019) c3 (tensin-1 isoform X7) c4(300639) c5(300640, 300641, 300642, 300643, 300644, 300645) c6( ) c7(T, aA, cd, q, mC, ad, eA, Na, aT, bj, K, ae, d, aW, Y, eZ, r, aq, af, iR, ck, es, mO, qJ, c, eM, TJ, iA, at, Dp, m, fL, B, hS); #c1(TNS1) c2(XP_011510024) c3(tensin-1 isoform X11) c4( ) c5(300646, 300647, 300648, 300649, 300650, 300651, 300652, 300653, 300654, 300655, 300656, 300657, 300658, 300659, 300660) c6( ) c7(T, aA, cd, q, mC, ad, eA, Na, aT, bj, K, ae, d, aW, Y, eZ, r, aq, af, iR, ck, es, mO, qJ, c, eM, TJ, iA, at, Dp, m, fL, B, hS); #c1(TNS2) c2(NP_056134) c3(tensin-like CI domain-containing phosphatase isoform 1) c4(185863) c5(185864, 185865, 185866, 185867, 185868, 185869) c6( ) c7(bl, d, aSO); #c1(TNS2) c2(NP_736610) c3 (tensin-like C1 domain-containing phosphatase isoform 2) c4(247330, 247331, 247332, 247333) c5(247334, 247335, 247336, 247337, 247338, 247339, 247340, 247341, 247342, 247343, 247344, 247345, 247346, 247347, 247348, 247349, 247350, 247351, 247352, 247353, 247354, 247355, 247356) c6( ) c7(bl, d, aSO); #c1(TNS2) c2(NP_938072) c3(tensin-like C1 domain-containing phosphatase isoform 3) c4( ) c5(259608, 259609) c6( ) c7(bl, d, aSO); #c1(TNS2) c2(XP_011536381) c3(tensin-like C1 domain-containing phosphatase isoform X3) c4( ) c5(333692) c6( ) c7(bl, d, aSO); #c1(TNS3) c2(XP_011513778) c3(tensin-3 isoform X2) c4( ) c5(306188, 306189, 306190, 306191, 306192, 306193, 306194, 306195, 306196) c6( ) c7(Ig, lJ, bzb, aT); #c1(TNS3) c2(XP_011513779) c3(tensin-3 isoform X3) c4(306197) c5(306198, 306199, 306200, 306201, 306202, 306203, 306204) c6( ) c7(Ig, lJ, bzb, aT); #c1(TNS3) c2(XP_011513780) c3(tensin-3 isoform X4) c4(306205) c5(306206) c6( ) c7(Ig, lJ, bzb, aT); #c1(TNS3) c2(XP_011513781) c3(tensin-3 isoform X5) c4(306207, 306208) c5(306209) c6( ) c7(Ig, lJ, bzb, aT); #c1(TNS3) c2(XP_011513785) c3(tensin-3 isoform X1) c4(306210, 306211) c5(306212, 306213, 306214, 306215, 306216, 306217, 306218, 306219, 306220, 306221, 306222, 306223, 306224, 306225, 306226, 306227, 306228, 306229, 306230, 306231, 306232) c6( ) c7(Ig, lJ, bzb, aT); #c1(TNS3) c2(XP_011513786) c3(tensin-3 isoform X6) c4(306233) c5(306234, 306235, 306236, 306237, 306238, 306239, 306240, 306241, 306242, 306243, 306244) c6( ) c7(Ig, lJ, bzb, aT); #c1(TNS4) c2(NP_116254) c3(tensin-4 precursor) c4(225986, 225987) c5(225988, 225989, 225990, 225991, 225992, 225993, 225994, 225995, 225996, 225997) c6( ) c7(ae, T, av, aE, Y, r, aA, aF, pd, bz, q, ad, cp); #c1(TNS4) c2(XP_005257801) c3(tensin-4 isoform X1) c4( ) c5(275365, 275366, 275367, 275368, 275369, 275370) c6( ) c7(ae, T, av, aE, Y, r, aA, aF, pd, bz, q, ad, cp); #c1(TNXB) c2(NP_061978) c3(tenascin-X isoform 1 precursor) c4(201431, 201432, 201433, 201434, 201435, 201436, 201437, 201438, 201439, 201440, 201441, 201442, 201443, 201444, 201445, 201446, 201447, 201448, 201449, 201450) c5(201451, 201452, 201453, 201454, 201455, 201456, 201457, 201458, 201459, 201460, 201461, 201462, 201463, 201464, 201465, 201466, 201467, 201468, 201469, 201470, 201471, 201472, 201473, 201474, 201475, 201476, 201477, 201478, 201479, 201480, 201481, 201482, 201483, 201484, 201485, 201486, 201487, 201488, 201489, 201490, 201491, 201492, 201493, 201494, 201495, 201496, 201497, 201498, 201499, 201500, 201501, 201502, 201503, 201504, 201505, 201506, 201507, 201508, 201509, 201510, 201511, 201512, 201513, 201514, 201515, 201516, 201517, 201518, 201519, 201520, 201521, 201522, 201523, 201524, 201525, 201526, 201527, 201528, 201529, 201530, 201531, 201532, 201533, 201534, 201535, 201536, 201537, 201538, 201539, 201540, 201541, 201542, 201543, 201544, 201545, 201546, 201547, 201548, 201549, 201550, 201551, 201552, 201553, 201554, 201555, 201556, 201557, 201558, 201559, 201560, 201561, 201562, 201563, 201564, 201565, 201566, 201567, 201568, 201569, 201570) c6(201571, 201572, 201573, 201574) c7(T, cOA, Jy, ci, Eh, afb, qC, ib, wG, oj, adX, h, aSF, cOz, af, xQ, A, aW, oV, cr, eZ, aiz, cOy, Ew, W, adZ, uo, m, y, hS); #c1(TNXB) c2(NP_115859) c3(tenascin-X isoform 2) c4( ) c5(224338, 224339, 224340, 224341, 224342, 224343, 224344, 224345, 224346, 224347, 224348, 224349, 224350, 224351, 224352) c6(224353, 224354) c7(T, cOA, Jy, ci, Eh, afb, qC, ib, wG, oj, adX, h, aSF, cOz, af, xQ, A, aW, oV, cr, eZ, aiz, cOy, Ew, W, adZ, uo, m, y, hS); #c1(TOB1) c2(NP_001230806) c3(protein Tob1 isoform 1) c4(86752) c5(86753, 86754, 86755, 86756, 86757, 86758, 86759, 86760, 86761, 86762) c6( ) c7(dP, ov, T, kh, av, aW, mF, aT, NI, aA, aF, aOJ, bz); #c1(TOB2) c2(NP_057356) c3(protein Tob2) c4(190320, 190321) c5(190322, 190323, 190324, 190325, 190326, 190327, 190328, 190329, 190330, 190331, 190332, 190333) c6( ) c7(qj); #c1(TOLLIP) c2(NP_061882) c3(toll-interacting protein) c4(200918, 200919) c5(200920, 200921, 200922, 200923, 200924, 200925, 200926, 200927, 200928, 200929, 200930, 200931) c6( ) c7(apJ, ji, ac, C, wz); #c1(TOM1) c2(NP_001129201) c3(target of Myb protein 1 isoform 3) c4(51149) c5(51150, 51151, 51152, 51153, 51154) c6( ) c7(x, mU, cA, mW, mZ, mS, mY, bK, mV, mT, mX); #c1(TOM1) c2(NP_001129202) c3(target of Myb protein 1 isoform 4) c4(51155, 51156) c5(51157, 51158, 51159, 51160, 51161, 51162, 51163, 51164, 51165, 51166, 51167, 51168) c6( ) c7(x, mU, cA, mW, mZ, mS, mY, bK, mV, mT, mX); #c1(TOM1) c2(NP_001129204) c3(target of Myb protein 1 isoform 2) c4(51174) c5(51175, 51176, 51177, 51178, 51179, 51180, 51181, 51182) c6( ) c7(x, mU, cA, mW, mZ, mS, mY, bK, mV, mT, mX); #c1(TOM1) c2(NP_005479) c3(target of Myb protein 1 isoform 1) c4(156545, 156546, 156547, 156548) c5(156549, 156550, 156551, 156552, 156553, 156554, 156555, 156556, 156557, 156558, 156559, 156560, 156561, 156562, 156563, 156564, 156565, 156566) c6( ) c7(x, mU, cA, mW, mZ, mS, mY, bK, mV, mT, mX); #c1(TOM1) c2(XP_011528120) c3(target of Myb protein 1 isoform X1) c4(323200) c5( ) c6( ) c7(x, mU, cA, mW, mZ, mS, mY, bK, mV, mT, mX): #c1(TOM1) c2(XP_011528121) c3(target of Myb protein 1 isoform X2) c4(323201) c5(323202, 323203, 323204, 323205, 323206, 323207, 323208, 323209, 323210, 323211, 323212, 323213, 323214, 323215, 323216, 323217, 323218) c6( ) c7(x, mU, cA, mW, mZ, mS, mY, bK, mV, mT, mX); #c1(TOM1L1) c2(NP_005477) c3(TOM1-like protein 1) c4(156529, 156530, 156531) c5(156532, 156533, 156534, 156535, 156536, 156537, 156538, 156539, 156540, 156541, 156542, 156543, 156544) c6( ) c7(ad); #c1(TOM1L1) c2(XP_011522458) c3(TOM1-like protein 1 isoform X2) c4( ) c5(317085, 317086, 317087, 317088, 317089, 317090, 317091, 317092, 317093, 317094, 317095, 317096, 317097) c6( ) c7(ad); #c1(TOMM20) c2(NP_055580) c3(mitochondrial import receptor subunit TOM20 homolog) c4(182069) c5(182070, 182071, 182072, 182073, 182074, 182075) c6( ) c7(K); #c1(TOMM34) c2(NP_006800) c3(mitochondrial import receptor subunit TOM34) c4(168192) c5(168193, 168194, 168195, 168196, 168197, 168198) c6( ) c7(X, W); #c1(TOMM40) c2(NP_001122389) c3(mitochondrial import receptor subunit TOM40 homolog) c4(46593) c5(46594, 46595, 46596, 46597, 46598, 46599, 46600, 46601, 46602, 46603, 46604, 46605, 46606, 46607) c6( ) c7(af, sO, e, bO, aT, gl, bS, ct, ei, Z, aaO, fK, cbh, vi); #c1(TOMM70A) c2(NP_055635) c3(mitochondrial import receptor subunit TOM70) c4(182418, 182419) c5(182420, 182421, 182422, 182423, 182424, 182425, 182426, 182427, 182428, 182429, 182430, 182431) c6( ) c7(wR, cbG); #c1(TONSL) c2(NP_038460) c3(tonsoku-like protein) c4(176503, 176504) c5(176505, 176506, 176507, 176508, 176509, 176510, 176511, 176512, 176513, 176514, 176515, 176516, 176517) c6( ) c7(T, dD, eq, K, kI); #c1(TOP1) c2(NP_003277) c3(DNA topoisomerase 1) c4(137036) c5(137037, 137038, 137039, 137040, 137041, 137042) c6( ) c7(gV, T, os, fR, fV, fG, ha, fd, bB, n, oZ, IR, q, aue, ad, x, av, ft, gd, aT, oW, hn, dL, K, rz, lM, ae, eL, kl, d, hG, aW, Y, r, Qm, eF, cm, bz, pb, ck, eM, bE, nF, bk, dy, k, bt, B); #c1(TOP2A) c2(NP_001058) c3(DNA topoisomerase 2-alpha) c4(34445, 34446, 34447) c5(34448, 34449, 34450, 34451, 34452, 34453, 34454, 34455, 34456, 34457, 34458, 34459, 34460, 34461, 34462, 34463, 34464, 34465) c6( ) c7(bL, T, atC, e, if, eX, jh, aA, n, ci, hF, q, ad, er, eA, eJ, oB, ip, ft, x, aE, aeO, qK, aT, iv, eV, dL, hP, K, ey, hB, ae, eL, gj, kl, d, B, aeR, Jl, aW, Y, qt, ff, iw, apK, r, Ej, aF, en, g, t, cM, bz, auC, ck, Dy, gt, eM, au, cj, hR, m, k, bC, bt, X, jr); #c1(TOP2A) c2(XP_005257689) c3(DNA topoisomerase 2-alpha isoform X1) c4(275333) c5(275334, 275335, 275336, 275337, 275338, 275339) c6( ) c7(bL, T, atC, e, if, eX, jh, aA, n, ci, hF, q, ad, er, eA, eJ, oB, ip, ft, x, aE, aeO, qK, aT, iv, eV, dL, hP, K, ey, hB, ae, eL, gj, kl, d, B, aeR, Jl, aW, Y, qt, ff, iw, apK, r, Ej, aF, en, g, t, cM, bz, auC, ck, Dy, gt, eM, au, cj, hR, m, k, bC, bt, X, jr); #c1(TOP2B) c2(NP_001059) c3(DNA topoisomerase 2-beta) c4(34466, 34467, 34468) c5(34469, 34470, 34471, 34472, 34473, 34474, 34475, 34476, 34477, 34478, 34479, 34480, 34481, 34482, 34483, 34484, 34485, 34486, 34487, 34488, 34489) c6( ) c7(ck, ae, T, eM, Pt, iT, cE, EQ, cj, jj, as, cm, bt, bz, cd, ad); #c1(TOP2B) c2(XP_005265484) c3(DNA topoisomerase 2-beta isoform X1) c4(279558, 279559) c5(279560, 279561, 279562, 279563, 279564, 279565, 279566, 279567) c6( ) c7(ck, ae, T, eM, Pt, iT, cE, EQ, cj, jj, as, cm, bt, bz, cd, ad); #c1(TOP2B) c2(XP_011532359) c3(DNA topoisomerase 2-beta isoform X2) c4( ) c5(328532, 328533, 328534, 328535, 328536) c6( ) c7(ck, ae, T, eM, Pt, iT, cE, EQ, cj, jj, as, cm, bt, bz, cd, ad); #c1(TOP3A) c2(NP_004609) c3(DNA topoisomerase 3-alpha) c4(148150, 148151) c5(148152, 148153, 148154, 148155, 148156, 148157) c6( ) c7(bv, T, MR, aW, aT, h, dL, as, bt, eL); #c1(TOP3A) c2(XP_005256833) c3(DNA topoisomerase 3-alpha isoform X1) c4(274550) c5(274551, 274552, 274553, 274554, 274555, 274556, 274557) c6( ) c7(bv, T, MR, aW, aT, h, dL, as, bt, eL); #c1(TOP3A) c2(XP_011522302) c3(DNA topoisomerase 3-alpha isoform X2) c4(316986) c5(316987, 316988, 316989, 316990, 316991, 316992) c6( ) c7(bv, T, MR, aW, aT, h, dL, as, bt, eL); #c1(TOP3A) c2(XP_011522303) c3(DNA topoisomerase 3-alpha isoform X3) c4( ) c5(316993, 316994, 316995, 316996, 316997) c6( ) c7(bv, T, MR, aW, aT, h, dL, as, bt, eL); #c1(TOP3B) c2(NP_001269042) c3(DNA topoisomerase 3-beta-1) c4(100280, 100281, 100282, 100283, 100284, 100285) c5(100286, 100287, 100288, 100289, 100290, 100291, 100292, 100293, 100294, 100295, 100296, 100297, 100298, 100299, 100300, 100301, 100302, 100303, 100304, 100305, 100306) c6( ) c7(aw); #c1(TOP3B) c2(XP_005261870) c3(DNA topoisomerase 3-beta-1 isoform X2) c4( ) c5(277647, 277648, 277649, 277650, 277651, 277652) c6( ) c7(aw); #c1(TOP3B) c2(XP_011528786) c3(DNA topoisomerase 3-beta-1 isoform X3) c4(324113, 324114, 324115, 324116, 324117, 324118) c5(324119, 324120, 324121, 324122, 324123, 324124, 324125, 324126, 324127, 324128, 324129, 324130, 324131, 324132, 324133, 324134, 324135, 324136, 324137, 324138, 324139, 324140, 324141, 324142, 324143, 324144, 324145, 324146, 324147, 324148, 324149, 324150, 324151, 324152, 324153, 324154, 324155, 324156, 324157, 324158, 324159, 324160, 324161, 324162, 324163, 324164, 324165, 324166) c6( ) c7(aw); #c1(TOPBP1) c2(NP_008958) c3(DNA topoisomerase 2-binding protein 1) c4(169802) c5(169803, 169804, 169805, 169806, 169807, 169808) c6( ) c7(ID, T, aC, aE, aW, Ot, arS, ew, aT, cj, r, K, m, ci, g, t, q, BY); #c1(TOPORS) c2(NP_001182551) c3(E3 ubiquitin-protein ligase Topors isoform 2) c4(75032) c5(75033, 75034, 75035, 75036, 75037, 75038) c6( ) c7(ae, xX, eM, cOB, nw, ci, eF, nC, bz, ad); #c1(TOPORS) c2(NP_005793) c3(E3 ubiquitin-protein ligase Topors isoform 1) c4(159376) c5(159377, 159378, 159379, 159380, 159381, 159382) c6( ) c7(ae, xX, eM, cOB, nw, ci, eF, nC, bz, ad); #c1(TOR1A) c2(NP_000104) c3(torsin-(A precursor) c4(1801, 1802) c5(1803, 1804, 1805, 1806, 1807, 1808, 1809, 1810, 1811, 1812, 1813, 1814) c6( ) c7(cOC, OR, aj, j, bfD, aR, Wk, aU, aks, WI, K, Wm, bAm, an, rE, aH, bex, Wi, yL, ayn, aGM, HL, dg, hp, cOF, xw, bDO, cOD, aiO, cEz, cOE, aGO, vL, aUZ, aUR, aen, bAr, ps, qc); #c1(TORIAIP1) c2(NP_056417) c3(torsin-1A-interacting protein 1 isoform 2) c4(187511) c5(187512, 187513, 187514, 187515, 187516, 187517, 187518, 187519, 187520, 187521, 187522, 187523) c6( ) c7(aU, eW, xs); #c1(TORIAIP1) c2(XP_011507706) c3(torsin-1A-interacting protein 1 isoform X2) c4(297716) c5( ) c6( ) c7(aU, eW, xs); #c1(TORIAIP2) c2(NP_071742) c3 (interferon alpha responsive protein isoform a) c4(211902) c5(211903, 211904, 211905, 211906, 211907, 211908) c6( ) c7(aU); #c1(TORIAIP2) c2(NP_659471) c3(torsin-A-interacting protein 2 isoform b) c4(240091) c5(240092, 240093, 240094, 240095, 240096, 240097) c6( ) c7(aU); #c1(TOR1B) c2(NP_055321) c3(torsin-18 precursor) c4(180462, 180463) c5(180464, 180465, 180466, 180467, 180468, 180469, 180470, 180471, 180472, 180473, 180474, 180475) c6( ) c7(aU, ds, bDO, j); #c1(TOR2A) c2(NP_001078816) c3(prosalusin isoform a precursor) c4(37901) c5(37902, 37903, 37904, 37905, 37906, 37907, 37908, 37909) c6(37910, 37911) c7(dD, ea, dh, j, eq, aCQ, cm); #c1(TOR2A) c2(NP_001127902) c3(prosalusin isoform c precursor) c4(49445) c5(49446, 49447, 49448, 49449, 49450, 49451, 49452, 49453) c6(49454, 49455) c7(dD, ea, dh, j, eq, aCQ, cm); #c1(TOR2A) c2(NP_001127903) c3(prosalusin isoform d precursor) c4(49456) c5(49457, 49458, 49459, 49460, 49461, 49462) c6(49463) c7(dD, ea, dh, j, eq, aCQ, cm); #c1(TOR2A) c2(NP_569726) c3(prosalusin isoform b precursor) c4(233828) c5(233829, 233830, 233831, 233832, 233833, 233834, 233835, 233836, 233837) c6(233838, 233839) c7(dD, ea, dh, j, eq, aCQ, cm); #c1(TOR2A) c2(XP_011516857) c3(prosalusin isoform X3) c4( ) c5(310290, 310291) c6( ) c7(dD, ea, dh, j, eq, aCQ, cm); #c1(TOX) c2(NP_055544) c3(thymocyte selection-associated high mobility group box protein TOX) c4(181868, 181869) c5(181870, 181871, 181872, 181873, 181874, 181875, 181876, 181877, 181878, 181879, 181880, 181881) c6( ) c7(T, ac, qb, e, aw, EQ, aT, j, cm, fq, eu); #c1(TOX2) c2(NP_001092266) c3(TOX high mobility group box family member 2 isoform c) c4(39481, 39482) c5(39483, 39484, 39485, 39486, 39487, 39488, 39489, 39490, 39491, 39492, 39493, 39494, 39495, 39496) c6( ) c7(Gp, av, T, bz, cm); #c1(TOX2) c2(NP_001092267) c3(TOX high mobility group box family member 2 isoform a) c4(39497) c5(39498, 39499, 39500, 39501, 39502, 39503, 39504, 39505, 39506) c6( ) c7(Gp, av, T, bz, cm); #c1(TOX2) c2(NP_001092268) c3(TOX high mobility group box family member 2 isoform b) c4(39507) c5(39508, 39509, 39510, 39511, 39512, 39513) c6( ) c7(Gp, av, T, bz, cm); #c1(TOX2) c2(XP_005260643) c3(TOX high mobility group box family member 2 isoform X2) c4( ) c5(276761, 276762, 276763, 276764, 276765, 276766, 276767, 276768, 276769, 276770, 276771) c6( ) c7(Gp, av, T, bz, cm); #c1(TOX2) c2(XP_006723947) c3(TOX high mobility group box family member 2 isoform X1) c4(295551) c5( ) c6( ) c7(Gp, av, T, bz, cm); #c1(TOX3) c2(NP_001073899) c3(TOX high mobility group box family member 3 isoform 1) c4(36355) c5(36356, 36357, 36358, 36359, 36360, 36361) c6( ) c7(T, zQ, kP, c, PE, aW, yY, aeU, aT, cHd, cX, eS, bK, m, t, aed); #c1(TOX3) c2(NP_001139660) c3(TOX high mobility group box family member 3 isoform 2) c4(59629) c5(59630, 59631, 59632, 59633, 59634, 59635) c6( ) c7(T, zQ, kP, c, PE, aW, yY, aeU, aT, cHd, cX, eS, bK, m, t, aed); #c1(TOX4) c2(NP_001290452) c3(TOX high mobility group box family member 4 isoform 2) c4(115479, 115480) c5(115481, 115482, 115483, 115484, 115485, 115486, 115487, 115488, 115489, 115490, 115491, 115492) c6( ) c7(T, fM, bP, bE, r, bB, n, ci, cl, q); #c1(TOX4) c2(NP_055643) c3(TOX high mobility group box family member 4 isoform 1) c4(182501) c5(182502, 182503, 182504, 182505, 182506, 182507, 182508, 182509, 182510, 182511) c6( ) c7(T, fM, bP, bE, r, bB, n, ci, cl, q); #c1(TP53) c2(NP_000537) c3(cellular tumor antigen p53 isoform a) c4(12271, 12272, 12273, 12274, 12275, 12276, 12277, 12278) c5(12279, 12280, 12281, 12282, 12283, 12284, 12285, 12286, 12287, 12288, 12289, 12290, 12291, 12292, 12293, 12294, 12295, 12296, 12297, 12298, 12299, 12300, 12301, 12302, 12303, 12304, 12305, 12306, 12307, 12308, 12309, 12310, 12311, 12312, 12313, 12314, 12315, 12316, 12317, 12318, 12319, 12320, 12321, 12322, 12323, 12324, 12325, 12326, 12327, 12328, 12329, 12330, 12331, 12332, 12333, 12334, 12335, 12336, 12337, 12338, 12339, 12340, 12341, 12342, 12343, 12344, 12345, 12346, 12347, 12348, 12349, 12350, 12351, 12352, 12353, 12354, 12355, 12356, 12357, 12358, 12359, 12360, 12361, 12362, 12363, 12364, 12365, 12366, 12367, 12368, 12369, 12370, 12371, 12372, 12373, 12374, 12375, 12376, 12377, 12378, 12379, 12380, 12381, 12382, 12383, 12384, 12385, 12386, 12387, 12388, 12389, 12390) c6(12391, 12392, 12393, 12394, 12395, 12396, 12397) c7(uB, gh, T, os, cEi, ea, aeu, cOQ, Vd, ci, aAo, jO, bfG, aJa, YD, GK, amR, aEI, bv, xy, aE, lv, ajv, auB, SV, aoK, dV, cPe, iv, hu, zy, aiJ, cp, aa, li, cPk, yo, cOV, ZH, cOf, iE, Ls, az, Az, QB, nY, CT, dg, PQ, zC, zE, lk, bfw, fT, cOG, iA, cPc, nB, dc, lp, avZ, bkv, cj, cX, k, fy, CE, cjG, YK, bxC, iB, aO, ht, RC, aj, auA, cd, br, cOP, aoc, cty, aex, rC, KN, bUP, lG, bkP, Gw, ey, bl, Dz, on, YT, aJb, bmF, pl, YI, aig, MD, Lh, ge, ahJ, fe, pm, iN, cPt, iO, bbz, ok, bkn, asl, dB, eD, bcd, pV, E, avr, qF, aCD, bkE, Po, aJJ, jp, Dh, fZ, aA, ol, bWZ, cz, yp, lu, AO, Eh, rd, q, aHy, zn, RU, bkJ, akU, qK, cPa, kf, rO, jj, ch, cOR, dL, iF, wr, gP, NE, nO, bfz, FO, cPh, iy, auX, lo, eS, cl, bkp, gQ, pb, cfZ, pa, rR, cdd, ac, iM, iQ, RW, nG, WU, aWQ, aor, aws, aps, bt, rA, On, gV, Mb, qz, iW, jh, Hn, KO, oZ, dG, Gn, fu, cHs, ceY, OE, buc, ad, afb, hX, iC, cPs, avp, Yt, avu, wD, yt, vJ, Ot, avs, oW, bj, iY, lQ, wm, cPn, ahK, aoZ, apw, MG, BO, cW, ff, gK, gC, nh, aeV, brp, Sm, iV, bz, amS, auH, aeH, Pk, aed, ne, rk, jr, bsJ, lC, bM, BA, iu, aEn, Fv, aKu, e, if, bB, cL, lN, cOU, aon, asr, sD, brq, ip, x, aif, cGK, ws, Ma, aeO, Gb, bFU, ja, us, azm, aNZ, aJu, bc, Qe, xQ, eG, anr, aeR, dH, Jl, hl, kt, Qm, cKZ, NR, as, eF, ir, cm, aHd, lt, bw, bEu, qJ, eY, lt, Kv, ii, vA, cON, mx, iS, hh, cOJ, in, ik, Th, Vj, sc, dD, gU, Qj, afA, lT, wu, atC, zt, aDT, n, oY, nA, Mo, b, no, cfF, Hx, Do, jz, amJ, gd, aaj, aYf, hn, eV, apD, aYF, pi, aeJ, hi, bUj, ia, aMC, A, RT, cPb, kj, hT, PX, AQ, Xg, hQ, GA, yq, sE, aJP, YE, bP, aQn, PI, agl, ajn, hL, hj, at, nI, pg, SZ, eo, cB, B, bmX, cug, AS, dA, eX, v, Qo, cPi, V, aYh, eJ, Yn, hW, wN, BJ, eO, AJ, aHs, bPF, avt, iZ, ae, jf, Lu, Yw, cOT, vT, aBq, ev, auc, bvJ, Qr, axW, Lf, bbs, qL, acB, aYz, bu, apx, LV, KQ, t, ho, Px, vk, eL, ck, kP, lI, cwA, aGi, cOy, au, gX, anD, oX, Gg, auN, zu, cOK, vi, HQ, nZ, hl, Dd, aBz, ml, cOH, eq, Fc, Tg, bkq, asi, aop, hd, CJ, Db, py, ds, cPI, hD, Ti, vc, bkw, bby, anN, nJ, aqa, Yj, d, auY, je, hG, et, dN, aoW, aWR, anW, gW, Z, dr, aVz, bF, gk, csl, afd, cOY, pP, Hs, aMy, QC, rH, y, kS, sH, cOO, pC, aC, FR, Js, azf, hV, al, aIN, CH, axk, qj, lR, Qp, Vp, afr, gJ, bnj, u, aEX, Xc, Bc, rJ, pX, bW, Qk, M, bJK, ayX, DO, K, aDZ, eL, avn, Us, aci, bPf, bn, pr, aEI, qS, wf, LZ, col, buS, aB, apm, g, pL, cvf, ve, QA, apB, iP, aAW, ou, bDd, aJw, AM, nF, U, PV, QF, cPd, dW, Hb, m, cPq, cPr, QO, sw, gp, zX, OI, NT, fN, cek, qy, aw, Qh, zP, JW, asS, biS, ft, bkm, lt, Tf, blp, Nn, kH, cU, aOM, By, Jn, ZN, KV, Ha, dx, awV, cPy, FZ, CN, aJL, ks, agF, YF, jT, cM, fs, XT, Zj, Ld, kn, ar, kT, kz, anZ, bWq, Tk, bo, auR, Lq, azr, zl, nz, lB, Pr, it, Jo, WL, aGn, fM, avm, boq, fG, aoE, jx, wo, hz, j, btX, acn, iI, pd, eA, J, o, er, XX, amg, MR, lg, aAF, yg, qv, aAv, bq, Fg, sb, avg, xR, Y, bEc, ayU, r, en, wA, aoG, aER, cPo, iK, bN, chr, QG, YV, sn, c, auo, Dy, hA, bei, bbA, wR, cS, np, dy, EY, KY, LS, gx, CX, kh, GI, akd, id, bkL, oA, bs, auq, bS, bkM, agH, OH, hF, XM, buj, iJ, Jm, fU, dP, fX, io, hP, ji, Yi, WW, lU, BN, lR, aus, anv, xs, eL, cr, aJI, gE, VI, bow, iR, KM, aAr, Bv, beC, Ew, Lj, asq, AR, bC, hE, G, jW, auW, pf, YU, qx, WV, zh, cPv, mF, aR, aQo, aJM, lS, dY, Ou, bnt, KP, apj, MO, pk, bXt, Zm, akV, zQ, bnh, aAu, aW, La, ahV, cOW, eM, iT, aep, uJ, gn, jM, Xu, QS, cZ, cPx, ajm, aEj, bui, Or, aBx, Bm, Wb, awr, fr, ct, qN, ai, eW, co, BI, hw, aMT, ic, h, dL, pj, hB, ask, Xh, aOU, cue, Gc, Df, Lg, Ck, YO, aiB, iw, aq, kp, Hu, mo, Ca, dw, op, aoO, oU, Gq, Qy, bkQ, cPj, Qu, hk, Vy, bm, gw, uz, oh, rx, cOL, ati, hR, YH, eQ, anx, ZZ, adG, cn, bL, cPp, eR, cPg, cjn, yk, WT, ew, tc, hy, aHa, bBs, azn, cOl, bkG, ga, mj, cOZ, nv, Vr, aoA, qp, afQ, ahL, aT, aJy, dz, Fx, Tj, ckx, af, qw, Uf, cPm, s, iU, oV, cPu, aJI, akY, f, brm, OC, sS, wg, nM, pZ, aGo, cPf, cPz, aJY, bko, aJQ, im, cOS, ava, VB, hS, bLx, Kd, Ke, Zl, aD, YM, cOX, Fb, OH, jE, aFJ, bkO, qC, av, l, F, ib, Rq, oz, sO, NS, Yo, azq, nI, cPw, cOM, bua, Od, fb, pq, iL, avQ, qt, Hq, eZ, yy, Ej, Xj, aF, aJT, Hp, LB, es, eU, RV, aoR, pc, kg, bE, WK, auw, aJz, aS, MC, adg, X, Ex); #c1(TP53) c2(NP_001119585) c3(cellular tumor antigen p53 isoform c) c4(44/79) c5(44180, 44181, 44182, 44183, 44184, 44185, 44186, 44187, 44188, 44189, 44190, 44191, 44192, 44193, 44194, 44195, 44196) c6(44197, 44198, 44199, 44200) c7(uB, gh, T, os, cEi, ea, aeu, cOQ, Vd, ci, aAo, jO, bfG, aJa, YD, GK, amR, aEI, bv, xy, aE, lv, ajv, auB, SV, aoK, dV, cPe, iv, hu, zy, aiJ, cp, aa, li, cPk, yo, cOV, ZH, cOf, iE, Ls, az, Az, QB, nY, CT, dg, PQ, zC, zE, lk, bfw, fT, cOG, iA, cPc, nB, dc, lp, avZ, bkv, cj, cX, k, fy, cE, cjG, YK, bxC, iB, aO, ht, RC, aj, auA, cd, br, cOP, aoc, cty, aex, rC, KN, bUP, lG, bkP, Gw, ey, bl, Dz, on, YT, aJb, bmF, pl, YI, aig, MD, Lh, ge, ahJ, fe, pm, iN, cPt, iO, bbz, ok, bkn, asl, dB, eD, bcd, pV, E, avr, qF, aCD, bkE, Po, aJJ, jp, Dh, fZ, aA, ol, bWZ, cz, yp, lu, AD, Eh, rd, q, aHy, zn, RU, bkJ, akU, qK, cPa, kf, rO, jj, ch, cOR, dL, iF, wr, gP, NE, nO, bfz, fO, cPh, iy, aux, lo, eS, cl, bkp, gQ, pb, cfZ, pa, rR, cdd, ac, iM, iQ, RW, nG, WU, aWQ, aor, aws, aps, bt, rA, On, gV, Mb, qz, iW, jh, Hn, KO, oZ, dG, Gn, fu, cHs, ceY, OE, buc, ad, afb, hX, iC, cPs, avp, Yt, avu, wD, yt, vJ, Ot, avs, oW, bj, iY, lQ, wm, cPn, ahK, aoZ, apw, MG, BO, cW, ff, gK, gC, nh, aeV, brp, Sm, iV, bz, amS, auH, aeH, Pk, aed, ne, rk, jr, bsJ, lC, bM, BA, iu, aEn, Fv, aKu, e, if, bB, cL, lN, cOU, aon, asr, sD, brq, ip, x, aif, cGK, ws, Ma, aeO, Gb, bFU, ja, us, azm, aNZ, aJu, bc, De, xQ, eG, anr, aeR, dH, Jl, hl, kt, Qm, cKZ, NR, as, eF, ir, cm, aHd, Qt, bw, bEu, qJ, eY, lt, Kv, ii, vA, cON, mx, iS, hh, cOJ, in, ik, Th, Vj, sc, dD, gU, Qj, afA, lT, wu, atC, zt, aDT, n, oY, nA, Mo, b, no, cfF, Hx, Do, jz, amJ, gd, aaj, aYf, hn, eV, apD, aYF, pi, aeJ, hi, bUj, ia, aMC, A, RT, cPb, kj, hT, PX, AQ, Xg, hQ, GA, yq, sE, aJP, YE, bP, aQn, PI, agl, ajn, hL, hj, at, nI, pg, SZ, eo, cB, B, bmX, cug, AS, dA, eX, v, Qo, cPi, V, aYh, eJ, Yn, hW, wN, BJ, eO, AJ, aHs, bPF, avt, iZ, ae, jf, Lu, Yw, cOT, vT, aBq, ev, auc, bvJ, Qr, axW, Lf, bbs, qL, acB, aYz, bu, apx, LV, KQ, t, ho, Px, vk, eL, ck, kP, lI, cwA, aGi, cOy, au, gX, anD, oX, Gg, auN, zu, cOK, vi, HQ, nZ, hl, Dd, aBz, mO, cOH, eq, Fc, Tg, bkq, asi, aop, hd, CJ, Db, py, ds, cPI, HD, Ti, vc, bkw, bby, anN, nJ, aqa, Yj, d, auY, je, hG, et, dN, aoW, aWR, anW, gW, Z, dr, aVz, bF, gk, csl, afd, cOY, pP, Hs, aMy, QC, rH, y, kS, sH, cOO, pC, aC, FR, Js, azf, hV, al, aIN, CH, axk, qj, lR, Qp, Vp, afr, gJ, bnj, u, aEX, Xc, Bc, rJ, pX, bW, Qk, M, bJK, ayX, DO, K, aDZ, eL, avn, Us, aci, bPf, bn, pr, aEI, qS, wf, LZ, col, buS, aB, apm, g, pL, cvf, ve, QA, apB, iP, aAW, ou, bDd, aJw, AM, nF, U, PV, QF, cPd, dW, Hb, m, cPq, cPr, QO, sw, gp, zX, OI, NT, fN, cek, qy, aw, Qh, zP, JW, asS, biS, ft, bkm, lt, Tf, blp, Nn, kH, cU, aOM, By, Jn, ZN, KV, Ha, dx, awV, cPy, FZ, CN, aJL, ks, agF, YF, jT, cM, fs, XT, Zj, Ld, kn, ar, kT, kz, anZ, bWq, Tk, bo, auR, Lq, azr, zl, nz, lB, Pr, it, Jo, WL, aGn, fM, avm, boq, fG, aOE, jx, wo, hz, j, btX, acn, iI, pd, eA, J, o, er, XX, amg, MR, lg, aAF, yg, qv, aAv, bq, Fg, sb, avg, xR, Y, bEc, ayU, r, en, wA, aoG, aER, cPo, iK, bN, chr, QG, YV, sn, c, auo, Dy, hA, bei, bbA, wR, cS, np, dy, EY, KY, LS, gx, CX, kh, GI, akd, id, bkL, oA, bs, auq, bS, bkM, agH, OH, hF, XM, buj, iJ, Jm, fU, dP, fX, io, hP, ji, Yi, WW, lU, BN, lR, aus, anv, xs, eH, cr, aJI, gE, VI, bow, iR, KM, aAr, Bv, beC, Ew, Lj, asq, AR, bC, hE, G, jW, auW, pf, YU, qx, WV, zh, cPv, mF, aR, aQo, aJM, lS, dY, Ou, bnt, KP, apj, MO, pk, bXt, Zm, akV, zQ, bnh, aAu, aW, La, ahV, cOW, eM, iT, aep, uJ, gn, jM, Xu, QS, cZ, cPx, ajm, aEj, bui, Or, aBx, Bm, Wb, awr, fr, ct, qN, ai, eW, co, BI, hw, aMT, ic, h, dL, pj, hB, ask, Xh, aOU, cue, Gc, Df, Lg, Ck, YO, aiB, iw, aq, kp, Hu, mo, Ca, dw, op, aoO, oU, Gq, Qy, bkQ, cPj, Qu, hk, Vy, bm, gw, uz, oh, rx, cOL, ati, HR, YH, eQ, anx, ZZ, adG, cn, bL, cPp, eR, cPg, cjn, yk, WT, ew, tc, hy, aHa, bBs, azn, col, bkG, ga, mj, cOZ, nv, Vr, aoA, qp, afQ, ahL, aT, aJy, dz, Fx, Tj, ckx, af, qw, Uf, cPm, s, iI, oV, cPu, aJI, akY, f, brm, OC, sS, wg, nM, pZ, aGo, cPf, cPz, aJY, bko, aJQ, im, cOS, ava, VB, hS, bLx, Kd, Ke, Zl, aD, YM, cOX, Fb, oH, jE, aFJ, bkO, qC, av, l, F, ib, Rq, oz, sO, NS, Yo, azq, nI, cPw, cOM, bua, Od, fb, pq, iL, avQ, qt, Hq, eZ, yy, Ej, Xj, aF, aJT, Hp, LB, es, eU, RV, aoR, pc, kg, bE, WK, auw, aJz, aS, MC, adg, X, Ex); #c1(TP53) c2(NP_001119586) c3(cellular tumor antigen p53 isoform b) c4(44201) c5(44202, 44203, 44204, 44205, 44206, 44207, 44208, 44209, 44210, 44211, 44212, 44213, 44214, 44215, 44216, 44217, 44218, 44219, 44220, 44221, 44222, 44223, 44224, 44225, 44226, 44227, 44228, 44229, 44230, 44231, 44232, 44233, 44234, 44235, 44236, 44237, 44238, 44239) c6(44240, 44241, 44242, 44243, 44244, 44245, 44246, 44247) c7(uB, gh, T, os, cEi, ea, aeu, cOQ, Vd, ci, aAo, jO, bfG, aJa, YD, GK, amR, aEI, bv, xy, aE, lv, ajv, auB, SV, aoK, dV, cPe, iv, hu, zy, aiJ, cp, aa, li, cPk, yo, cOV, ZH, cOf, iE, Ls, az, Az, QB, nY, CT, dg, PQ, zC, zE, lk, bfw, fT, cOG, iA, cPc, nB, dc, lp, avZ, bkv, cj, cX, k, fy, cE, cjG, YK, bxC, iB, aO, ht, RC, aj, auA, cd, br, cOP, aoc, cty, aeX, rC, KN, bUP, lG, bkP, Gw, ey, bl, Dz, on, YT, aJb, bmF, pl, YI, aig, MD, Lh, ge, ahJ, fe, pm, iN, cPt, iO, bbz, ok, bkn, asl, dB, eD, bcd, pV, E, avr, qF, aCD, bkE, Po, aJJ, jp, Dh, fZ, aA, ol, bWZ, cz, yp, lu, AO, Eh, rd, q, aHy, zn, RU, bkJ, akU, qK, cPa, kf, rO, jj, ch, cOR, dl, iF, wr, gP, NE, nO, bfz, fO, cPh, iy, aux, lo, eS, cl, bkp, gQ, pb, cfZ, pa, rR, cdd, ac, iM, iQ, RW, nG, WU, aWQ, aor, aws, aps, bt, rA, On, gV, Mb, qz, iW, jh, Hn, KO, oZ, dG, Gn, fu, cHs, ceY, OE, buc, ad, afb, hX, iC, cPs, avp, Yt, avu, wD, yt, vJ, Ot, avs, oW, bj, iY, lQ, wm, cPn, ahK, aoZ, apw, MG, BO, cW, ff, gK, gC, nh, aeV, brp, Sm, iV, bz, amS, auH, aeH, Pk, aed, ne, rk, jr, bsJ, lC, bM, BA, iu, aEn, Fv, aKu, e, if, bB, cL, lN, cOU, aon, asr, sD, brq, ip, x, aif, cGK, ws, Ma, aeO, Gb, bFU, ja, us, azm, aNZ, aJu, bc, Qe, xQ, eG, anr, aeR, dH, Jl, hl, kt, Qm, cKZ, NR, as, eF, ir, cm, aHd, Qt, bw, bEu, qJ, eY, lt, Kv, ii, vA, cON, mx, iS, hh, cOJ, in, ik, Th, Vj, sc, dD, gU, Qj, afA, lT, wu, atC, zt, aDT, n, oY, nA, Mo, b, no, cfF, Hx, Do, jz, amJ, gd, aaj, aYf, hn, eV, apD, aYF, pi, aeJ, hi, bUj, ia, aMC, A, RT, cPb, kj, hT, PX, AQ, Xg, hQ, GA, yq, sE, aJP, YE, bP, aQn, PI, agl, ajn, hL, hj, at, nL, pg, SZ, eo, cB, B, bmX, cug, AS, dA, eX, v, Qo, cPi, V, aYh, eJ, Yn, hW, wN, BJ, eO, AJ, aHs, bPF, avt, iZ, ae, jf, Lu, Yw, cOT, vT, aBq, ev, auc, bvJ, Qr, axW, Lf, bbs, qL, acB, aYz, bu, apx, LV, KQ, t, ho, Px, vk, el, ck, kP, lI, cwA, aGi, cLy, au, gX, anD, oX, Gg, auN, zu, cOK, vi, HQ, nZ, hl, Dd, aBz, ml, cOH, eq, Fc, Tg, bkq, asi, aop, hd, CJ, Db, py, ds, cPI, hD, Ti, vc, bkw, bby, anN, nJ, aqa, Yj, d, auY, je, hG, et, dN, aoW, aWR, anW, gW, Z, dr, aVz, bF, gk, csl, afd, cOY, pP, Hs, aMy, QC, rH, y, kS, sH, cOO, pC, aC, fR, Js, azf, hV, al, aIN, CH, axk, qj, lR, Qp, Vp, afr, gJ, bnj, u, aEX, Xc, Bc, rJ, pX, bW, Qk, M, bJK, ayX, DO, K, aDZ, eL, avn, Us, aci, bPf, bn, pr, aEI, qS, wf, LZ, col, buS, aB, apm, g, pL, cvf, ve, QA, apB, iP, aAW, ou, bDd, aJw, AM, nF, U, PV, QF, cPd, dW, Hb, m, cPq, cPr, QO, sw, gp, zX, OI, NT, fN, cek, qy, aw, Qh, zP, JW, asS, biS, ft, bkm, lt, Tf, blp, Nn, kH, cU, aOM, By, Jn, ZN, KV, Ha, dx, awV, cPy, FZ, CN, aJL, ks, agF, YF, jT, cM, fs, XT, Zj, Ld, kn, ar, kT, kz, anZ, bWq, Tk, bo, auR, Lq, azr, zl, nz, lB, Pr, it, Jo, WL, aGn, fM, avm, boq, fG, aoE, jx, wo, hz, j, btX, acn, iI, pd, eA, J, o, er, XX, amg, MR, lg, aAF, yg, qv, aAv, bq, Fg, sb, avg, xR, Y, bEc, ayU, r, en, wA, aoG, aER, cPo, iK, bN, chr, QG, YV, sn, c, auo, Dy, hA, bei, bbA, wR, cS, np, dy, EY, KY, LS, gx, CX, kh, GI, akd, id, bkL, oA, bs, auq, bS, bkM, agH, OH, hF, XM, buj, iJ, Jm, fU, dP, fX, io, hP, ji, Yi, WW, lU, BN, lR, aus, anv, xs, eH, cr, aJI, gE, VI, bow, iR, KM, aAr, Bv, beC, Ew, Lj, asq, AR, bC, hE, G, jW, auW, pf, YU, qx, WV, zh, cPv, mF, aR, aQo, aJM, lS, dY, Ou, bnt, KP, apj, MO, pk, bXt, Zm, akV, zQ, bnh, aAu, aW, La, ahV, cOW, eM, iT, aep, uJ, gn, jM, Xu, QS, cZ, cPx, ajm, aEj, bui, Or, aBx, Bm, Wb, awr, fr, ct, qN, ai, eW, co, BI, hw, aMT, ic, h, dL, pj, hB, ask, Xh, aOU, cue, Gc, Df, Lg, Ck, YO, aiB, iw, aq, kp, Hu, mo, Ca, dw, op, aoO, oU, Gq, Qy, bkQ, cPj, Qu, hk, Vy, bm, gw, uz, oh, rx, cOL, ati, hR, YH, eQ, anx, ZZ, adG, cn, bL, cPp, eR, cPg, cjn, yk, WT, ew, tc, hy, aHa, bBs, azn, cOl, bkG, ga, mj, cOZ, nv, Vr, aoA, qp, afQ, ahL, aT, aJy, dz, Fx, Tj, ckx, af, qw, Uf, cPm, s, iU, oV, cPu, aJI, akY, f, brm, OC, sS, wg, nM, pZ, aGo, cPf, cPz, aJY, bko, aJQ, im, cOS, ava, VB, hS, bLx, Kd, Ke, Zl, aD, YM, cOX, Fb, oH, jE, aFJ, bkO, qC, av, l, F, ib, Rq, oz, SO, NS, Yo, azq, nI, cPw, cOM, bua, Od, fb, pq, iL, avQ, qt, Hq, eZ, yy, Ej, Xj, aF, aJT, Hp, LB, es, eU, RV, aoR, pc, kg, bE, WK, auw, aJz, aS, MC, adg, X, Ex); #c1(TP53) c2(NP_001119587) c3(cellular tumor antigen p53 isoform d) c4(44248) c5( ) c6( ) c7(uB, gh, T, os, cEi, ea, aeu, cOQ, Vd, ci, aAo, jO, bfG, aJa, YD, GK, amR, aEI, bv, xy, aE, lv, ajv, auB, SV, aoK, dV, cPe, iv, hu, zy, aiJ, cp, aa, li, cPk, yo, cOV, ZH, cOf, iE, Ls, az, Az, QB, nY, CT, dg, PQ, zC, zE, lk, bfw, fT, cOG, iA, cPc, nB, dc, Ip, avZ, bkv, cj, cX, k, fy, cE, cjG, YK, bxC, iB, aO, ht, RC, aj, auA, cd, br, cOP, aoc, cty, aex, rC, KN, bUP, lG, bkP, Gw, ey, bl, Dz, on, YT, aJb, bmF, pl, YI, aig, MD, Lh, ge, ahJ, fe, pm, iN, cPt, iO, bbz, ok, bkn, asl, dB, eD, bcd, pV, E, avr, qF, aCD, bkE, Po, aJJ, jp, Dh, fZ, aA, ol, bWZ, cz, yp, lu, AO, Eh, rd, q, aHy, zn, RU, bkJ, akU, qK, cPa, kf, rO, jj, ch, cOR, dl, iF, wr, gP, NE, nO, bfz, fO, cPh, iy, aux, lo, eS, cl, bkp, gQ, pb, cfZ, pa, rR, cdd, ac, iM, iQ, RW, nG, WU, aWQ, aor, aws, aps, bt, rA, On, gV, Mb, qz, iW, jh, Hn, KO, oZ, dG, Gn, fu, cHs, ceY, OE, buc, ad, afb, hX, iC, cPs, avp, Yt, avu, wD, yt, vJ, Ot, avs, oW, bj, iY, lQ, wm, cPn, ahK, aoZ, apw, MG, BO, cW, ff, gK, gC, nh, aeV, brp, Sm, iV, bz, amS, auH, aeH, Pk, aed, ne, rk, jr, bsJ, lC, bM, BA, iu, aEn, Fv, aKu, e, if, bB, cL, lN, cOU, aon, asr, sD, brq, ip, x, aif, cGK, ws, Ma, aeO, Gb, bFU, ja, us, azm, aNZ, aJu, bc, Qe, xQ, eG, anr, aeR, dH, Jl, hl, kt, Qm, cKZ, NR, as, eF, ir, cm, aHd, Qt, bw, bEu, qJ, eY, lt, Kv, ii, vA, cON, mx, iS, hh, cOJ, in, ik, Th, Vj, sc, dD, gU, Qj, afA, lT, wu, atC, zt, aDT, n, oY, nA, Mo, b, no, cfF, Hx, Do, jz, amJ, gd, aaj, aYf, hn, eV, apD, aYF, pi, aeJ, hi, bUj, ia, aMC, A, RT, cPb, kj, hT, PX, AQ, Xg, hQ, GA, yq, sE, aJP, YE, bP, aQn, PI, agl, ajn, hL, hj, at, nL, pg, SZ, eo, cB, B, bmX, cug, AS, dA, eX, v, Qo, cPi, V, aYh, eJ, Yn, hW, wN, BJ, eO, AJ, aHs, bPF, avt, iZ, ae, jf, Lu, Yw, cOT, vT, aBq, ev, auc, bvJ, Qr, axW, Lf, bbs, qL, acB, aYz, bu, apx, LV, KQ, t, ho, Px, vk, el, ck, kP, lI, cwA, aGi, cLy, au, gX, anD, oX, Gg, auN, zu, cOK, vi, HQ, nZ, hl, Dd, aBz, ml, cOH, eq, Fc, Tg, bkq, asi, aop, hd, CJ, Db, py, ds, cPI, hD, Ti, vc, bkw, bby, anN, nJ, aqa, Yj, d, auY, je, hG, et, dN, aoW, aWR, anW, gW, Z, dr, aVz, bF, gk, csl, afd, cOY, pP, Hs, aMy, QC, rH, y, kS, sH, cOO, pC, aC, fR, Js, azf, hV, al, aIN, CH, axk, qj, lR, Qp, Vp, afr, gJ, bnj, u, aEX, Xc, Bc, rJ, pX, bW, Qk, M, bJK, ayX, DO, K, aDZ, eL, avn, Us, aci, bPf, bn, pr, aEI, qS, wf, LZ, col, buS, aB, apm, g, pL, cvf, ve, QA, apB, iP, aAW, ou, bDd, aJw, AM, nF, U, PV, QF, cPd, dW, Hb, m, cPq, cPr, QO, sw, gp, zX, OI, NT, fN, cek, qy, aw, Qh, zP, JW, asS, biS, ft, bkm, lt, Tf, blp, Nn, kH, cU, aOM, By, Jn, ZN, KV, Ha, dx, awV, cPy, FZ, CN, aJL, ks, agF, YF, jT, cM, fs, XT, Zj, Ld, kn, ar, kT, kz, anZ, bWq, Tk, bo, auR, Lq, azr, zl, nz, lB, Pr, it, Jo, WL, aGn, fM, avm, boq, fG, aoE, jx, wo, hz, j, btX, acn, iI, pd, eA, J, o, er, XX, amg, MR, lg, aAF, yg, qv, aAv, bq, Fg, sb, avg, xR, Y, bEc, ayU, r, en, wA, aoG, aER, cPo, iK, bN, chr, QG, YV, sn, c, auo, Dy, hA, bei, bbA, wR, cS, np, dy, EY, KY, LS, gx, CX, kh, GI, akd, id, bkL, oA, bs, auq, bS, bkM, agH, OH, hF, XM, buj, iJ, Jm, fU, dP, fX, io, hP, ji, Yi, WW, lU, BN, lR, aus, anv, xs, eH, cr, aJI, gE, VI, bow, iR, KM, aAr, Bv, beC, Ew, Lj, asq, AR, bC, hE, G, jW, auW, pf, YU, qx, WV, zh, cPv, mF, aR, aQo, aJM, lS, dY, Ou, bnt, KP, apj, MO, pk, bXt, Zm, akV, zQ, bnh, aAu, aW, La, ahV, cOW, eM, iT, aep, uJ, gn, jM, Xu, QS, cZ, cPx, ajm, aEj, bui, Or, aBx, Bm, Wb, awr, fr, ct, qN, ai, eW, co, BI, hw, aMT, ic, h, dL, pj, hB, ask, Xh, aOU, cue, Gc, Df, Lg, Ck, YO, aiB, iw, aq, kp, Hu, mo, Ca, dw, op, aoO, oU, Gq, Qy, bkQ, cPj, Qu, hk, Vy, bm, gw, uz, oh, rx, cOL, ati, hR, YH, eQ, anx, ZZ, adG, cn, bL, cPp, eR, cPg, cjn, yk, WT, ew, tc, hy, aHa, bBs, azn, cOl, bkG, ga, mj, cOZ, nv, Vr, aoA, qp, afQ, ahL, aT, aJy, dz, Fx, Tj, ckx, af, qw, Uf, cPm, s, iU, oV, cPu, aJI, akY, f, brm, OC, sS, wg, nM, pZ, aGo, cPf, cPz, aJY, bko, aJQ, im, cOS, ava, VB, hS, bLx, Kd, Ke, Zl, aD, YM, cOX, Fb, OH, jE, aFJ, bkO, qC, av, l, F, ib, Rq, oz, sO, NS, Yo, azq, nI, cPw, cOM, bua, Od, fb, pq, iL, avQ, qt, Hq, eZ, yy, Ej, Xj, aF, aJT, Hp, LB, es, eU, RV, aoR, pc, kg, bE, WK, auw, aJz, aS, MC, adg, X, Ex); #c1(TP53) c2(NP_001119588) c3(cellular tumor antigen p53 isoform e) c4( ) c5(44249, 44250, 44251, 44252, 44253, 44254, 44255, 44256, 44257, 44258, 44259, 44260, 44261, 44262) c6(44263, 44264, 44265) c7(uB, gh, T, os, cEi, ea, aeu, cOQ, Vd, ci, aAo, jO, bfG, aJa, YD, GK, amR, aEI, bv, xy, aE, lv, ajv, auB, SV, aoK, dV, cPe, iv, hu, zy, aiJ, cp, aa, li, cPk, yo, cOV, ZH, cOf, iE, Ls, az, Az, QB, nY, CT, dg, PQ, zC, zE, lk, bfw, fT, cOG, iA, cPc, nB, dc, lp, avZ, bkv, cj, cX, k, fy, cE, cjG, YK, bxC, iB, aa, ht, RC, aj, auA, cd, br, cOP, aoc, cty, aeX, rC, KN, bUP, lG, bkP, Gw, By, bl, Dz, on, YT, aJb, bmF, pl, YI, aig, MD, Lh, ge, ahJ, fe, pm, iN, cPt, iO, bbz, ok, bkn, asl, dB, eD, bcd, pV, E, avr, qF, aCD, bkE, Po, aJJ, jp, Dh, fZ, aA, ol, bWZ, cz, yp, lu, AO, Eh, rd, q, aHy, zn, RU, bkJ, akU, qK, cPa, kf, rO, jj, ch, cOR, dl, iF, wr, gP, NE, nO, bfz, fO, cPh, iy, aux, lo, eS, cl, bkp, gQ, pb, cfZ, pa, rR, cdd, ac, iM, iQ, RW, nG, WU, aWQ, aor, aws, aps, bt, rA, On, gV, Mb, qz, iW, jh, Hn, KO, oZ, dG, Gn, fu, cHs, ceY, OE, buc, ad, afb, hX, iC, cPs, avp, Yt, avu, wD, yt, vJ, Ot, avs, oW, bj, iY, lQ, wm, cPn, ahK, aoZ, apw, MG, BO, cW, ff, gK, gC, nh, aeV, brp, Sm, iV, bz, amS, auH, acH, Pk, aed, ne, rk, jr, bsJ, lC, bM, BA, iu, aEn, Fv, aKu, e, if, bB, cL, lN, cOU, aon, asr, sD, brq, ip, x, aif, cGK, ws, Ma, aeO, Gb, bFU, ja, us, azm, aNZ, aJu, bc, De, xQ, eG, anr, aeR, dH, Jl, hl, kt, Qm, cKZ, NR, as, ef, ir, cm, aHd, Qt, bw, bEu, qJ, eY, lt, Kv, ii, vA, cON, mx, iS, hh, cOJ, in, ik, Th, Vj, sc, dD, gU, Qj, afA, lT, wu, atC, zt, aDT, n, oY, nA, Mo, b, no, cfF, Hx, Do, jz, amJ, gd, aaj, aYf, hn, eV, apD, aYF, pi, aeJ, hi, bUj, ia, aMC, A, RT, cPb, kj, hT, PX, AQ, Xg, hQ, GA, yq, sE, aJP, YE, bP, aQn, PI, agl, ajn, hL, hj, at, nL, pg, SZ, eo, cB, B, bmX, cug, AS, dA, eX, v, Qo, cPi, V, aYh, eJ, Yn, hW, wN, BJ, eO, AJ, aHs, bPF, avt, iZ, ae, jf, Lu, Yw, cOT, vT, aBq, ev, auc, bvJ, Qr, axW, Lf, bbs, qL, acB, aYz, bu, apx, LV, KQ, t, ho, Px, vk, el, ck, kP, lI, cwA, aGi, cLy, au, gX, anD, oX, Gg, auN, zu, cOK, vi, HQ, nZ, hl, Dd, aBz, mO, cOH, eq, Fc, Tg, bkq, asi, aop, hd, CJ, Db, py, ds, cPI, hD, Ti, vc, bkw, bby, anN, nJ, aqa, Yj, d, auY, je, hG, et, dN, aoW, aWR, anW, gW, Z, dr, aVz, bF, gk, csl, afd, cOY, pP, Hs, aMy, QC, rH, y, kS, sH, cOO, pC, aC, fR, Js, azf, hV, al, aIN, CH, axk, qj, lR, Qp, Vp, afr, gJ, bnj, u, aEX, Xc, Bc, rJ, pX, bW, Qk, M, bJK, ayX, DO, K, aDZ, eL, avn, Us, aci, bPf, bn, pr, aEI, qS, wf, LZ, col, buS, aB, apm, g, pL, cvf, ve, QA, apB, iP, aAW, ou, bDd, aJw, AM, nF, U, PV, QF, cPd, dW, Hb, m, cPq, cPr, QO, sw, gp, zX, OI, NT, fN, cek, qy, aw, Qh, zP, JW, asS, biS, ft, bkm, lt, Tf, blp, Nn, kH, cU, aOM, By, Jn, ZN, KV, Ha, dx, awV, cPy, FZ, CN, aJL, ks, agF, YF, jT, cM, fs, XT, Zj, Ld, kn, ar, kT, kz, anZ, bWq, Tk, bo, auR, Lq, azr, zl, nz, lB, Pr, it, Jo, WL, aGn, fM, avm, boq, fG, aoE, jx, wo, hz, j, btX, acn, iI, pd, eA, J, o, er, XX, amg, MR, lg, aAF, yg, qv, aAv, bq, Fg, sb, avg, xR, Y, bEc, ayU, r, en, wA, aoG, aER, cPo, iK, bN, chr, QG, YV, sn, c, auo, Dy, hA, bei, bbA, wR, cS, np, dy, EY, KY, LS, gx, CX, kh, GI, akd, id, bkL, oA, bs, auq, bS, bkM, agH, OH, hF, XM, buj, iJ, Jm, fU, dP, fX, io, hP, ji, Yi, WW, lU, BN, lR, aus, anv, xs, eH, cr, aJI, gE, VI, bow, iR, KM, aAr, Bv, beC, Ew, Lj, asq, AR, bC, hE, G, jW, auW, pf, YU, qx, WV, zh, cPv, mF, aR, aQo, aJM, lS, dY, Ou, bnt, KP, apj, MO, pk, bXt, Zm, akV, zQ, bnh, aAu, aW, La, ahV, cOW, eM, iT, aep, uJ, gn, jM, Xu, QS, cZ, cPx, ajm, aEj, bui, Or, aBx, Bm, Wb, awr, fr, ct, qN, ai, eW, co, BI, hw, aMT, ic, h, dL, pj, hB, ask, Xh, aOU, cue, Gc, Df, Lg, Ck, YO, aiB, iw, aq, kp, Hu, mo, Ca, dw, op, aoO, oU, Gq, Qy, bkQ, cPj, Qu, hk, Vy, bm, gw, uz, oh, rx, cOL, ati, hR, YH, eQ, anx, ZZ, adG, cn, bL, cPp, eR, cPg, cjn, yk, WT, ew, tc, hy, aHa, bBs, azn, cOl, bkG, ga, mj, cOZ, nv, Vr, aoA, qp, afQ, ahL, aT, aJy, dz, Fx, Tj, ckx, af, qw, Uf, cPm, s, iU, oV, cPu, aJI, akY, f, brm, OC, sS, wg, nM, pZ, aGo, cPf, cPz, aJY, bko, aJQ, im, cOS, ava, VB, hS, bLx, Kd, Ke, Zl, aD, YM, cOX, Fb, oH, jE, aFJ, bkO, qC, av, l, F, ib, Rq, oz, sO, NS, Yo, azq, nI, cPw, cOM, bua, Od, fb, pq, iL, avQ, qt, Hq, eZ, yy, Ej, Xj, aF, aJT, Hp, LB, es, eU, RV, aoR, pc, kg, bE, WK, auw, aJz, aS, MC, adg, X, Ex); #c1(TP53) c2(NP_001119590) c3(cellular tumor antigen p53 isoform g) c4(44266) c5(44267) c6( ) c7(uB, gh, T, os, cEi, ea, aeu, cOQ, Vd, ci, aAo, jO, bfG, aJa, YD, GK, amR, aEI, bv, xy, aE, lv, ajv, auB, SV, aoK, dV, cPe, iv, hu, zy, aiJ, cp, aa, li, cPk, yo, cOV, ZH, cOf, iE, Ls, az, Az, QB, nY, CT, dg, PQ, zC, zE, lk, bfw, fT, cOG, iA, cPc, nB, dc, lp, avZ, bkv, cj, cX, k, fy, cE, cjG, YK, bxC, iB, aO, ht, RC, aj, auA, cd, br, cOP, aoc, cty, aex, rC, KN, bUP, lG, bkP, Gw, ey, bl, Dz, on, YT, aJb, bmF, pl, YI, aig, MD, Lh, ge, ahJ, fe, pm, iN, cPt, iO, bbz, ok, bkn, asl, dB, eD, bcd, pV, E, avr, qF, aCD, bkE, Po, aJJ, jp, Dh, fZ, aA, ol, bWZ, cz, yp, lu, AD, Eh, rd, q, aHy, zn, RU, bkJ, akU, qK, cPa, kf, rO, jj, ch, cOR, dL, iF, wr, gP, NE, nO, bfz, fO, cPh, iy, aux, lo, eS, cl, bkp, gQ, pb, cfZ, pa, rR, cdd, ac, iM, iQ, RW, nG, WU, aWQ, aor, aws, aps, bt, rA, On, gV, Mb, qz, iW, jh, Hn, KO, oZ, dG, Gn, fu, cHs, ceY, OE, buc, ad, afb, hX, iC, cPs, avp, Yt, avu, wD, yt, vJ, Ot, avs, oW, bj, iY, lQ, wm, cPn, ahK, aoZ, apw, MG, BO, cW, ff, gK, gC, nh, aeV, brp, Sm, iV, bz, amS, auH, aeH, Pk, aed, ne, rk, jr, bsJ, lC, bM, BA, iu, aEn, Fv, aKu, e, if, bB, cL, lN, cOU, aon, asr, sD, brq, ip, x, aif, cGK, ws, Ma, aeO, Gb, bFU, ja, us, azm, aNZ, aJu, bc, Qe, xQ, eG, anr, aeR, dH, Jl, hl, kt, Qm, cKZ, NR, as, eF, ir, cm, aHd, Qt, bw, bEu, qJ, eY, lt, Kv, ii, vA, cON, mx, iS, hh, cOJ, in, ik, Th, Vj, sc, dD, gU, Qj, afA, lT, wu, atC, zt, aDT, n, oY, nA, Mo, b, no, cfF, Hx, Do, jz, amJ, gd, aaj, aYf, hn, eV, apD, aYF, pi, aeJ, hi, bUj, ia, aMC, A, RT, cPb, kj, hT, PX, AQ, Xg, hQ, GA, yq, SE, aJP, YE, bP, aQn, PI, agl, ajn, hL, hj, at, nL, pg, SZ, eo, cB, B, bmX, cug, AS, dA, eX, v, Do, cPi, V, aYh, eJ, Yn, hW, wN, BJ, eO, AJ, aHs, bPF, avt, iZ, ae, jf, Lu, Yw, cOT, vT, aBq, ev, auc, bvJ, Qr, axW, Lf, bbs, qL, acB, aYz, bu, apx, LV, KQ, t, ho, Px, vk, eL, ck, kP, lI, cwA, aGi, cOy, au, gX, anD, oX, Gg, auN, zu, cOK, vi, HQ, nZ, hl, Dd, aBz, ml, cOH, eq, Fc, Tg, bkq, asi, aop, hd, CJ, Db, py, ds, cPI, hD, Ti, vc, bkw, bby, anN, nJ, aqa, Yj, d, auY, je, hG, et, dN, aoW, aWR, anW, gW, Z, dr, aVz, bF, gk, csl, afd, cOY, pP, Hs, aMy, QC, rH, y, kS, sH, cOO, pC, aC, fR, Js, azf, hV, al, aIN, CH, axk, qj, lR, Qp, Vp, afr, gJ, bnj, u, aEX, Xc, Bc, rJ, pX, bW, Qk, M, bJK, ayX, DO, K, aDZ, eL, avn, Us, aci, bPf, bn, pr, aEI, qS, wf, LZ, col, buS, aB, apm, g, pL, cvf, ve, QA, apB, iP, aAW, ou, bDd, aJw, AM, nF, U, PV, QF, cPd, dW, Hb, m, cPq, cPr, QO, sw, gp, zX, OI, NT, fN, cek, qy, aw, Qh, zP, JW, asS, biS, ft, bkm, lt, Tf, blp, Nn, kH, cU, aOM, By, Jn, ZN, KV, Ha, dx, awV, cPy, FZ, CN, aJL, ks, agF, YF, jT, cM, fs, XT, Zj, Ld, kn, ar, KT, kz, anZ, bWq, Tk, bo, auR, Lq, azr, zl, nz, lB, Pr, it, Jo, WL, aGn, fM, avm, boq, fG, aoE, jx, wo, hz, j, btX, acn, iI, pd, eA, J, o, er, XX, amg, MR, lg, aAF, yg, qv, aAv, bq, Fg, sb, avg, xR, Y, bEc, ayU, r, en, wA, aoG, aER, cPo, iK, bN, chr, QG, YV, sn, c, auo, Dy, hA, bei, bbA, wR, cS, np, dy, EY, KY, LS, gx, CX, kh, GI, akd, id, bkL, oA, bs, auq, bS, bkM, agH, OH, hF, XM, buj, iJ, Jm, fU, dP, fX, io, hP, ji, Yi, WW, lU, BN, lR, aus, anv, xs, eH, cr, aJI, gE, VI, bow, iR, KM, aAr, Bv, beC, Ew, Lj, asq, AR, bC, hE, G, jW, auW, pf, YU, qx, WV, zh, cPv, mF, aR, aQo, aJM, lS, dY, Ou, bnt, KP, apj, MO, pk, bXt, Zm, akV, zQ, bnh, aAu, aW, La, ahV, cOW, eM, iT, aep, uJ, gn, jM, Xu, QS, cZ, cPx, ajm, aEj, bui, Or, aBx, Bm, Wb, awr, fr, ct, qN, ai, eW, co, BI, hw, aMT, ic, h, dL, pj, hB, ask, Xh, aOU, cue, Gc, Df, Lg, Ck, YO, aiB, iw, aq, kp, Hu, mo, Ca, dw, op, aoO, oU, Gq, Qy, bkQ, cPj, Qu, hk, Vy, bm, gw, uz, oh, rx, cOL, ati, hR, YH, eQ, anx, ZZ, adG, cn, bL, cPp, eR, cPg, cjn, yk, WT, ew, tc, hy, aHa, bBs, azn, cOl, bkG, ga, mj, cOZ, nv, Vr, aoA, qp, afQ, ahL, aT, aJy, dz, Fx, Tj, ckx, af, qw, Uf, cPm, s, iU, oV, cPu, aJI, akY, f, brm, OC, sS, wg, nM, pZ, aGo, cPf, cPz, aJY, bko, aJQ, im, cOS, ava, VB, hS, bLx, Kd, Ke, Zl, aD, YM, cOX, Fb, oH, jE, aFJ, bkO, qC, av, l, F, ib, Rq, oz, sO, NS, Yo, azq, nI, cPw, cOM, bua, Od, fb, pq, iL, avQ, qt, Hq, eZ, yy, Ej, Xj, aF, aJT, Hp, LB, es, eU, RV, aoR, pc, kg, bE, WK, auw, aJz, aS, MC, adg, X, Ex); #c1(TP53) c2(NP_001263625) c3(cellular tumor antigen p53 isoform i) c4( ) c5(97289, 97290, 97291, 97292, 97293, 97294, 97295, 97296, 97297, 97298, 97299, 97300, 97301, 97302, 97303) c6(97304, 97305, 97306, 97307) c7(uB, gh, T, os, cEi, ea, aeu, cOQ, Vd, ci, aAo, jO, bfG, aJa, YD, GK, amR, aEI, bv, xy, aE, Iv, ajv, auB, SV, aoK, dV, cPe, iv, hu, zy, aiJ, cp, aa, li, cPk, yo, cOV, ZH, cOf, iE, Ls, az, Az, QB, nY, CT, dg, PQ, zC, zE, lk, bfw, fT, cOG, iA, cPc, nB, dc, lp, avZ, bkv, cj, cX, k, fy, cE, cjG, YK, bxC, iB, aO, ht, RC, aj, auA, cd, br, cOP, aoc, cty, aex, rC, KN, bUP, lG, bkP, Gw, ey, bl, Dz, on, YT, aJb, bmF, pl, YI, aig, MD, Lh, ge, ahJ, fe, pm, iN, cPt, iO, bbz, ok, bkn, asl, dB, eD, bcd, pV, E, avr, qF, aCD, bkE, Po, aJJ, jp, Dh, fZ, aA, ol, bWZ, cz, yp, lu, AD, Eh, rd, q, aHy, zn, RU, bkJ, akU, qK, cPa, kf, rO, jj, ch, cOR, dL, iF, wr, gP, NE, nO, bfz, fO, cPh, iy, aux, lo, eS, cl, bkp, gQ, pb, cfZ, pa, rR, cdd, ac, iM, iQ, RW, nG, WU, aWQ, aor, aws, aps, bt, rA, On, gV, Mb, qz, iW, jh, Hn, KD, oZ, dG, Gn, fu, cHs, ceY, DE, buc, ad, afb, hX, iC, cPs, avp, Yt, avu, wD, yt, vJ, Ot, avs, oW, bj, iY, lQ, wm, cPn, ahK, aoZ, apw, MG, BO, cW, ff, gK, gC, nh, aeV, brp, Sm, iV, bz, amS, auH, aeH, Pk, aed, ne, rk, jr, bsJ, lC, bM, BA, iu, aEn, Fv, aKu, e, if, bB, cL, lN, cOU, aon, asr, sD, brq, ip, x, aif, cGK, ws, Ma, aeO, Gb, bFU, ja, us, azm, aNZ, aJu, bc, Qe, xQ, eG, anr, aeR, dH, Jl, hl, kt, Qm, cKZ, NR, as, eF, ir, cm, aHd, Qt, bw, bEu, qJ, eY, lt, Kv, ii, vA, cON, mx, iS, hh, cOJ, in, ik, Th, Vj, sc, dD, gU, Qj, afA, lT, wu, atC, zt, aDT, n, oY, nA, Mo, b, no, cfF, Hx, Do, jz, amJ, gd, aaj, aYf, hn, eV, apD, aYF, pi, aeJ, hi, bUj, ia, aMC, A, RT, cPb, kj, hT, PX, AQ, Xg, hQ, GA, yq, sE, aJP, YE, bP, aQn, PI, agl, ajn, hL, hj, at, nL, pg, SZ, eo, cB, B, bmX, cug, AS, dA, eX, v, Qo, cPi, V, aYh, eJ, Yn, hW, wN, BJ, eO, AJ, aHs, bPF, avt, iZ, ae, jf, Lu, Yw, cOT, vT, aBq, ev, auc, bvJ, Qr, axW, Lf, bbs, qL, acB, aYz, bu, apx, LV, KQ, t, ho, Px, vk, el, ck, kP, lI, cwA, aGi, cLy, au, gX, anD, oX, Gg, auN, zu, cOK, vi, HQ, nZ, hl, Dd, aBz, ml, cOH, eq, Fc, Tg, bkq, asi, aop, hd, CJ, Db, py, ds, cPI, HD, Ti, vc, bkw, bby, anN, nJ, aqa, Yj, d, auY, je, hG, et, dN, aoW, aWR, anW, gW, Z, dr, aVz, bF, gk, csl, afd, cOY, pP, Hs, aMy, QC, rH, y, kS, sH, cOO, pC, aC, FR, Js, azf, hV, al, aIN, CH, axk, qj, lR, Qp, Vp, afr, gJ, bnj, u, aEX, Xc, Bc, rJ, pX, bW, Qk, M, bJK, ayX, DO, K, aDZ, eL, avn, Us, aci, bPf, bn, pr, aEI, qS, wF, LZ, col, buS, aB, apm, g, pL, cvf, ve, QA, apB, iP, aAW, ou, bDd, aJw, AM, nF, U, PV, QF, cPd, dW, Hb, m, cPq, cPr, QO, sw, gp, zX, OI, NT, fN, cek, qy, aw, Ch, zP, JW, asS, biS, ft, bkm, lt, Tf, blp, Nn, kH, cU, aOM, By, Jn, ZN, KV, Ha, dx, awV, cPy, FZ, CN, aJL, ks, agF, YF, jT, cM, fs, XT, Zj, Ld, kn, ar, kT, kz, anZ, bWq, Tk, bo, auR, Lq, azr, zl, nz, lB, Pr, it, Jo, WL, aGn, fM, avm, boq, fG, aoE, jx, wo, hz, j, btX, acn, iI, pd, eA, J, o, er, XX, amg, MR, lg, aAF, yg, qv, aAv, bq, Fg, sb, avg, xR, Y, bEc, ayU, r, en, wA, aoG, aER, cPo, iK, bN, chr, QG, YV, sn, c, auo, Dy, hA, bei, bbA, wR, cS, np, dy, EY, KY, LS, gx, CX, kh, GI, akd, id, bkL, oA, bs, auq, bS, bkM, agH, OH, hF, XM, buj, iJ, Jm, fU, dP, fX, io, hP, ji, Yi, WW, lU, BN, lR, aus, anv, xs, eH, cr, aJI, gE, VI, bow, iR, KM, aAr, Bv, beC, Ew, Lj, asq, AR, bC, hE, G, jW, auW, pf, YU, qx, WV, zh, cPv, mF, aR, aQo, aJM, lS, dY, Ou, bnt, KP, apj, MO, pk, bXt, Zm, akV, zQ, bnh, aAu, aW, La, ahV, cOW, eM, iT, aep, uJ, gn, jM, Xu, QS, cZ, cPx, ajm, aEj, bui, Or, aBx, Bm, Wb, awr, fr, ct, qN, ai, eW, co, BI, hw, aMT, ic, h, dL, pj, hB, ask, Xh, aOU, cue, Gc, Df, Lg, Ck, YO, aiB, iw, aq, kp, Hu, mo, Ca, dw, op, aoO, oU, Gq, Qy, bkQ, cPj, Qu, hk, Vy, bm, gw, uz, oh, rx, cOL, ati, hR, YH, eL, anx, ZZ, adG, cn, bL, cPp, eR, cPg, cjn, yk, WT, ew, tc, hy, aHa, bBs, azn, cOl, bkG, ga, mj, cOZ, nv, Vr, aoA, qp, afQ, ahL, aT, aJy, dz, Fx, Tj, ckx, af, qw, Uf, cPm, s, iU, oV, cPu, aJI, akY, f, brm, OC, sS, wg, nM, pZ, aGo, cPf, cPz, aJY, bko, aJQ, im, cOS, ava, VB, hS, bLx, Kd, Ke, Zl, aD, YM, cOX, Fb, oH, jE, aFJ, bkO, qC, av, l, F, ib, Rq, oz, sO, NS, Yo, azq, nI, cPw, cOM, bua, Od, fb, pq, iL, avQ, qt, Hq, eZ, yy, Ej, Xj, aF, aJT, Hp, LB, es, eU, RV, aoR, pc, kg, bE, WK, auw, aJz, aS, MC, adg, X, Ex); #c1(TP53) c2(NP_001263626) c3(cellular tumor antigen p53 isoform j) c4(97308) c5(97309, 97310, 97311, 97312, 97313, 97314) c6(97315) c7(uB, gh, T, os, cEi, ea, aeu, cOQ, Vd, ci, aAo, jO, bfG, aJa, YD, GK, amR, aEI, bv, xy, aE, lv, ajv, auB, SV, aoK, dV, cPe, iv, hu, zy, aiJ, cp, aa, li, cPk, yo, cOV, ZH, cOf, iE, Ls, az, Az, QB, nY, CT, dg, PQ, zC, zE, lk, bfw, fT, cOG, iA, cPc, nB, dc, lp, avZ, bkv, cj, cX, k, fy, cE, cjG, YK, bxC, iB, aO, ht, RC, aj, auA, cd, br, cOP, aoc, cty, aeX, rC, KN, bUP, lG, bkP, Gw, ey, bl, Dz, on, YT, aJb, bmF, pl, YI, aig, MD, Lh, ge, ahJ, fe, pm, iN, cPt, iO, bbz, ok, bkn, asl, dB, eD, bcd, pV, E, avr, qF, aCD, bkE, Po, aJJ, jp, Dh, fZ, aA, ol, bWZ, cz, yp, lu, AO, Eh, rd, q, aHy, zn, RU, bkJ, akU, qK, cPa, kf, rO, jj, ch, cOR, dl, iF, wr, gP, NE, nO, bfz, fO, cPh, iy, aux, lo, eS, cl, bkp, gQ, pb, cfZ, pa, rR, cdd, ac, iM, iQ, RW, nG, WU, aWQ, aor, aws, aps, bt, rA, On, gV, Mb, qz, iW, jh, Hn, KO, oZ, dG, Gn, fu, cHs, ceY, OE, buc, ad, afb, hX, iC, cPs, avp, Yt, avu, wD, yt, vJ, Ot, avs, oW, bj, iY, lQ, wm, cPn, ahK, aoZ, apw, MG, BO, cW, ff, gK, gC, nh, aeV, brp, Sm, iV, bz, amS, auH, aeH, Pk, aed, ne, rk, jr, bsJ, lC, bM, BA, iu, aEn, Fv, aKu, e, if, bB, cL, lN, cOU, aon, asr, sD, brq, ip, x, aif, cGK, ws, Ma, aeO, Gb, bFU, ja, us, azm, aNZ, aJu, bc, Qe, xQ, eG, anr, aeR, dH, Jl, hl, kt, Qm, cKZ, NR, as, eF, ir, cm, aHd, Qt, bw, bEu, qJ, eY, It, Kv, ii, vA, cON, mx, iS, hh, cOJ, in, ik, Th, Vj, sc, dD, gU, Qj, afA, lT, wu, atC, zt, aDT, n, oY, nA, Mo, b, no, cfF, Hx, Do, jz, amJ, gd, aaj, aYf, hn, eV, apD, aYF, pi, aeJ, hi, bUj, ia, aMC, A, RT, cPb, kj, hT, PX, AQ, Xg, hQ, GA, yq, sE, aJP, YE, bP, aQn, Pl, agl, ajn, hL, hj, at, nL, pg, SZ, eo, cB, B, bmX, cug, AS, dA, eX, v, Do, cPi, V, aYh, eJ, Yn, hW, wN, BJ, eO, AJ, aHs, bPF, avt, iZ, ae, jf, Lu, Yw, cOT, vT, aBq, ev, auc, bvJ, Qr, axW, Lf, bbs, qL, acB, aYz, bu, apx, LV, KQ, t, ho, Px, vk, el, ck, kP, lI, cwA, aGi, cLy, au, gX, anD, oX, Gg, auN, zu, cOK, vi, HQ, nZ, hl, Dd, aBz, ml, cOH, eq, Fc, Tg, bkq, asi, aop, hd, CJ, Db, py, ds, cPI, hD, Ti, vc, bkw, bby, anN, nJ, aqa, Yj, d, auY, je, hG, et, dN, aoW, aWR, anW, gW, Z, dr, aVz, bF, gk, csl, afd, cOY, pP, Hs, aMy, QC, rH, y, kS, sH, cOO, pC, aC, fR, Js, azf, hV, al, aIN, CH, axk, qj, lR, Qp, Vp, afr, gJ, bnj, u, aEX, Xc, Bc, rJ, pX, bW, Qk, M, bJK, ayX, DO, K, aDZ, eL, avn, Us, aci, bPf, bn, pr, aEI, qS, wf, LZ, col, buS, aB, apm, g, pL, cvf, ve, QA, apB, iP, aAW, ou, bDd, aJw, AM, nF, U, PV, QF, cPd, dW, Hb, m, cPq, cPr, QO, sw, gp, zX, OI, NT, fN, cek, qy, aw, Qh, zP, JW, asS, biS, ft, bkm, lt, Tf, blp, Nn, kH, cU, aOM, By, Jn, ZN, KV, Ha, dx, awV, cPy, FZ, CN, aJL, ks, agF, YF, jT, cM, fs, XT, Zj, Ld, kn, ar, KT, kz, anZ, bWq, Tk, bo, auR, Lq, azr, zl, nz, lB, Pr, it, Jo, WL, aGn, fM, avm, boq, fG, aoE, jx, wo, hz, j, btX, acn, iI, pd, eA, J, o, er, XX, amg, MR, lg, aAF, yg, qv, aAv, bq, Fg, sb, avg, xR, Y, bEc, ayU, r, en, wA, aoG, aER, cPo, iK, bN, chr, QG, YV, sn, c, auo, Dy, hA, bei, bbA, wR, cS, np, dy, EY, KY, LS, gx, CX, kh, GI, akd, id, bkL, oA, bs, auq, bS, bkM, agH, OH, hF, XM, buj, iJ, Jm, fU, dP, fX, io, hP, ji, Yi, WW, lU, BN, lR, aus, anv, xs, eH, cr, aJI, gE, VI, bow, iR, KM, aAr, Bv, beC, Ew, Lj, asq, AR, bC, hE, G, jW, auW, pf, YU, qx, WV, zh, cPv, mF, aR, aQo, aJM, lS, dY, Ou, bnt, KP, apj, MO, pk, bXt, Zm, akV, zQ, bnh, aAu, aW, La, ahV, cOW, eM, iT, aep, uJ, gn, jM, Xu, QS, cZ, cPx, ajm, aEj, bui, Or, aBx, Bm, Wb, awr, fr, ct, qN, ai, eW, co, BI, hw, aMT, ic, h, dL, pj, hB, ask, Xh, aOU, cue, Gc, Df, Lg, Ck, YO, aiB, iw, aq, kp, Hu, mo, Ca, dw, op, aoO, oU, Gq, Qy, bkQ, cPj, Qu, hk, Vy, bm, gw, uz, oh, rx, cOL, ati, hR, YH, eQ, anx, ZZ, adG, cn, bL, cPp, eR, cPg, cjn, yk, WT, ew, tc, hy, aHa, bBs, azn, col, bkG, ga, mj, cOZ, nv, Vr, aoA, qp, afQ, ahL, aT, aJy, dz, Fx, Tj, ckx, af, qw, Uf, cPm, s, iU, oV, cPu, aJI, akY, f, brm, OC, sS, wg, nM, pZ, aGo, cPf, cPz, aJY, bko, aJQ, im, cOS, ava, VB, hS, bLx, Kd, Ke, Zl, aD, YM, cOX, Fb, OH, jE, aFJ, bkO, qC, av, l, F, ib, Rq, oz, SO, NS, Yo, azq, nI, cPw, cOM, bua, Od, fb, pq, iL, avQ, qt, Hq, eZ, yy, Ej, Xj, aF, aJT, Hp, LB, es, eL, RV, aoR, pc, kg, bE, WK, auw, aJz, aS, MC, adg, X, Ex); #c1(TP53AIP1) c2(NP_001182123) c3(p53-regulated apoptosis-inducing protein 1 isoform c) c4(74595) c5(74596, 74597, 74598, 74599, 74600, 74601) c6( ) c7(ae, T, av, FR, eM, az, aA, aq, aF, ci, K, bz, B, ad); #c1(TP53AIP1) c2(NP_001182124) c3(p53-regulated apoptosis-inducing protein 1 isoform b) c4(74602) c5(74603, 74604, 74605, 74606, 74607, 74608) c6( ) c7(ae, T, av, fR, eM, az, aA, aq, aF, ci, K, bz, B, ad); #c1(TP53BP1) c2(NP_001135451) c3(tumor suppressor p53-binding protein 1 isoform 2) c4(52943) c5(52944, 52945, 52946, 52947, 52948, 52949) c6( ) c7(T, kh, fM, aC, AS, if, ht, jh, aA, fd, n, hy, ci, qN, atV, mC, ad, eA, azn, hd, aE, afQ, aT, K, ae, Nr, d, iU, aW, oV, ff, eZ, ks, po, en, g, t, iK, bz, Nq, sS, kP, auV, nB, hk, cj, at, m, fL, bC, cE, AN); #c1(TP53BP1) c2(NP_001135452) c3(tumor suppressor p53-binding protein 1 isoform 1) c4(52950) c5(52951, 52952, 52953, 52954, 52955, 52956, 52957, 52958, 52959, 52960, 52961, 52962) c6( ) c7(T, kh, fM, aC, AS, if, ht, jh, aA, fd, n, hy, ci, qN, atV, mC, ad, eA, azn, hd, aE, afQ, aT, K, ae, Nr, d, iU, aW, oV, ff, eZ, ks, po, en, g, t, iK, bz, Nq, sS, kP, auV, nB, hk, cj, at, m, fL, bC, cE, AN); #c1(TP53BP1) c2(NP_005648) c3(tumor suppressor p53-binding protein 1 isoform 3) c4(158251) c5( ) c6( ) c7(T, kh, fM, aC, AS, if, ht, jh, aA, fd, n, hy, ci, qN, atV, mC, ad, eA, azn, hd, aE, afQ, aT, K, ae, Nr, d, iU, aW, oV, ff, eZ, ks, po, en, g, t, iK, bz, Nq, sS, kP, auV, nB, hk, cj, at, m, fL, bC, cE, AN); #c1(TP53BP1) c2(XP_011520287) c3 (tumor suppressor p53-binding protein 1 isoform X2) c4(315123) c5(315124, 315125, 315126, 315127, 315128, 315129) c6( ) c7(T, kh, fM, aC, AS, if, ht, jh, aA, fd, n, hy, ci, qN, atV, mC, ad, eA, azn, hd, aE, afQ, aT, K, ae, Nr, d, iU, aW, oV, ff, eZ, ks, po, en, g, t, iK, bz, Nq, sS, kP, auV, nB, hk, cj, at, m, fL, bC, cE, AN); #c1(TP53BP2) c2(NP_001026855) c3(apoptosis-stimulating of p53 protein 2 isoform 1) c4(29662) c5(29663, 29664, 29665, 29666, 29667, 29668, 29669, 29670) c6( ) c7(ck, T, ZH, af, aoc, CN, aW, jh, aA, hn, aF, hF, nJ, B, hX); #c1(TP53BP2) c2(XP_011542570) c3(apoptosis-stimulating of p53 protein 2 isoform X2) c4(341124) c5( ) c6( ) c7(ck, T, ZH, af, aoc, CN, aW, jh, aA, hn, aF, hF, nJ, B, hX); #c1(TP53BP2) c2(XP_011542571) c3(apoptosis-stimulating of p53 protein 2 isoform X1) c4(34/125) c5(34/126, 341127, 341128, 341129, 341130) c6( ) c7(ck, T, ZH, af, aoc, CN, aW, jh, aA, hn, aF, hF, nJ, B, hX); #c1(TP53BP2) c2(XP_011542572) c3(apoptosis-stimulating of p53 protein 2 isoform X3) c4( ) c5(341131, 341132, 341133, 341134, 341135, 341136) c6( ) c7(ck, T, ZH, af, aoc, CN, aW, jh, aA, hn, aF, hF, nJ, B, hX); #c1(TP53111) c2(NP_001245253) c3(tumor protein p53-inducible protein 11) c4(92852, 92853, 92854, 92855, 92856, 92857, 92858) c5(92859, 92860, 92861, 92862, 92863, 92864, 92865, 92866, 92867, 92868, 92869, 92870, 92871, 92872, 92873, 92874, 92875, 92876, 92877, 92878, 92879, 92880, 92881, 92882) c6( ) c7(aq, aF, B, aA); #c1(TP53111) c2(XP_005253286) c3(tumor protein p53-inducible protein 11 isoform X2) c4( ) c5(272380, 272381, 272382, 272383, 272384, 272385, 272386, 272387, 272388, 272389, 272390, 272391, 272392, 272393, 272394, 272395, 272396, 272397, 272398, 272399, 272400, 272401, 272402, 272403, 272404, 272405, 272406, 272407, 272408, 272409) c6( ) c7(aq, aF, B, aA); #c1(TP53113) c2(NP_612358) c3 (tumor protein p53-inducible protein 13 precursor) c4(236237, 236238) c5(236239, 236240, 236241, 236242, 236243, 236244, 236245, 236246, 236247, 236248, 236249, 236250) c6( ) c7(K); #c1(TP5313) c2(NP_001193731) c3(quinone oxidoreductase PIG3 isoform 2) c4(82523) c5(82524, 82525, 82526, 82527, 82528, 82529, 82530, 82531, 82532, 82533, 82534, 82535) c6( ) c7(T, fM, zQ, eM, aT, at, dL, ci, K, m, iK, ae, ad); #c1(TP5313) c2(NP_671713) c3(quinone oxidoreductase PIG3 isoform 1) c4(242134, 242135) c5(242136, 242137, 242138, 242139, 242140, 242141, 242142, 242143, 242144, 242145) c6( ) c7(T, fM, zQ, eM, aT, at, dL, ci, K, m, iK, ae, ad); #c1(TP53INP1) c2(NP_001129205) c3(tumor protein p53-inducible nuclear protein 1 isoform b) c4(51183) c5(51184, 51185, 51186, 51187, 51188, 51189, 51190, 51191, 51192, 51193, 51194) c6( ) c7(bL, ck, T, cc, aE, iA, aW, Y, aD, aT, aA, aF, wb, ci, K, fr, eA); #c1(TP53INP1) c2(XP_011515688) c3(tumor protein p53-inducible nuclear protein 1 isoform X1) c4(308523, 308524) c5(308525, 308526, 308527, 308528, 308529, 308530, 308531) c6( ) c7(bL, ck, T, cc, aE, iA, aW, Y, aD, aT, aA, aF, wb, ci, K, fr, eA); #c1(TP53INP2) c2(NP_067025) c3(tumor protein p53-inducible nuclear protein 2) c4(208478, 208479, 208480) c5(208481, 208482, 208483, 208484, 208485, 208486, 208487, 208488, 208489, 208490, 208491, 208492, 208493, 208494, 208495, 208496, 208497, 208498, 208499, 208500, 208501, 208502) c6( ) c7(T, aW, pQ, aeS, aT, jh, dL, ci, K, zZ, iK, eL); #c1(TP53RK) c2(NP_291028) c3(TP53-regulating kinase) c4(228902, 228903) c5(228904, 228905, 228906, 228907, 228908, 228909, 228910, 228911, 228912, 228913, 228914, 228915) c6( ) c7(T, cF, K); #c1(TP53TG3C) c2(NP_001192188) c3(TP53-target gene 3 protein) c4(82037, 82038) c5(82039, 82040, 82041, 82042, 82043, 82044, 82045, 82046, 82047, 82048, 82049, 82050, 82051, 82052, 82053, 82054, 82055, 82056, 82057, 82058, 82059, 82060, 82061, 82062, 82063, 82064, 82065, 82066, 82067, 82068, 82069, 82070, 82071, 82072) c6( ) c7(T); #c1(TP63) c2(NP_001108450) c3(tumor protein 63 isoform 2) c4(43200) c5(43201, 43202, 43203, 43204, 43205, 43206, 43207, 43208, 43209, 43210, 43211, 43212) c6( ) c7(aOR, bM, T, aC, Fv, if, ci, cPD, YD, ip, Na, aOS, aE, arA, pX, Eo, dV, K, hu, eL, Ur, li, iE, aW, cPB, are, az, Az, aOW, OZ, aEI, LZ, ark, g, eE, zE, eF, cm, eM, Jr, cPc, lq, nB, vA, nF, wK, cj, aPc, k, m, cE, wu, iB, zt, ht, Bm, n, qN, ld, aPb, BI, ft, amJ, PD, cU, h, dL, Dz, aOU, aOX, A, MD, ks, fe, po, aOT, op, Ye, Qy, Qu, eD, hR, at, eo, YH, it, B, lp, fM, yk, arN, AO, V, pd, q, er, Zp, akG, aOY, cPE, hW, nX, afQ, qK, aT, rO, rD, Nc, Tj, aPa, avt, aOV, ae, le, xR, Y, Lf, afS, r, en, bu, t, Ud, iK, sS, QG, kP, cPA, zu, KY, rA, id, atS, jh, Hn, zU, adg, cPC, ad, aOZ, av, hd, ib, Db, lQ, wm, fb, nJ, aeI, ff, eH, cr, eL, asV, Ej, bz, atb, dr, LB, z, afT, y, xg, iu); #c1(TP63) c2(NP_001108451) c3(tumor protein 63 isoform 3) c4(43213) c5(43214, 43215, 43216, 43217, 43218, 43219) c6( ) c7(aOR, bM, T, aC, Fv, if, ci, cPD, YD, ip, Na, aOS, aE, arA, pX, Eo, dV, K, hu, eL, Ur, li, iE, aW, cPB, are, az, Az, aOW, OZ, aEI, LZ, ark, g, BE, ZE, eF, cm, eM, Jr, cPc, lq, nB, vA, nF, wK, cj, aPc, k, m, cE, wu, iB, zt, ht, Bm, n, qN, ld, aPb, BI, ft, amJ, PD, cU, h, dL, Dz, aOU, aOX, A, MD, ks, fe, po, aOT, op, Ye, Qy, Qu, eD, hR, at, eo, YH, it, B, lp, fM, yk, arN, AO, V, pd, q, er, Zp, akG, aOY, cPE, hW, nX, afQ, qK, aT, rO, rD, Nc, Tj, aPa, avt, aOV, ae, le, xR, Y, Lf, afS, r, en, bu, t, Ud, iK, sS, QG, kP, cPA, zu, KY, rA, id, atS, jh, Hn, zU, adg, cPC, ad, aOZ, av, hd, ib, Db, lQ, wm, fb, nJ, aeQ, ff, eH, cr, eZ, asV, Ej, bz, atb, dr, LB, z, afT, y, xg, iu); #c1(TP63) c2(NP_001108452) c3(tumor protein 63 isoform 4) c4(43220, 43221, 43222, 43223) c5(43224, 43225, 43226, 43227, 43228, 43229, 43230, 43231, 43232, 43233, 43234, 43235) c6( ) c7(aOR, bM, T, aC, Fv, if, ci, cPD, YD, ip, Na, aOS, aE, arA, pX, Eo, dV, K, hu, eL, Ur, li, iE, aW, cPB, are, az, Az, aOW, OZ, aEI, LZ, ark, g, eE, ZE, eF, cm, eM, Jr, cPc, lq, nB, vA, nF, wK, cj, aPc, k, m, cE, wu, iB, zt, ht, Bm, n, qN, ld, aPb, BI, ft, amJ, PD, cU, h, dL, Dz, aOU, aOX, A, MD, ks, fe, po, aOT, op, Ye, Qy, Qu, eD, hR, at, eo, YH, it, B, lp, fM, yk, arN, AO, V, pd, q, er, Zp, akG, aOY, cPE, hW, nX, afQ, qK, aT, rO, rD, Nc, Tj, aPa, avt, aOV, ae, le, xR, Y, Lf, afS, r, en, bu, t, Ud, iK, sS, QG, kP, cPA, zu, KY, rA, id, atS, jh, Hn, zU, adg, cPC, ad, aOZ, av, hd, ib, Db, lQ, wm, fb, nJ, aeQ, ff, eH, cr, eZ, asV, Ej, bz, atb, dr, LB, z, afT, y, xg, iu); #c1(TP63) c2(NP_001108453) c3(tumor protein 63 isoform 5) c4(43236) c5(43237, 43238, 43239, 43240, 43241, 43242, 43243, 43244, 43245, 43246, 43247, 43248) c6( ) c7(aOR, bM, T, aC, Fv, if, ci, cPD, YD, ip, Na, aOS, aE, arA, pX, Eo, dV, K, hu, eL, Ur, li, iE, aW, cPB, are, az, Az, aOW, OZ, aEI, LZ, ark, g, eE, zE, eF, cm, eM, Jr, cPc, lq, nB, vA, nF, wK, cj, aPc, k, m, cE, wu, iB, zt, ht, Bm, n, qN, ld, aPb, BI, ft, amJ, PD, cU, h, dL, Dz, aOU, aOX, A, MD, ks, fe, po, aOT, op, Ye, Qy, Qu, eD, hR, at, eo, YH, it, B, lp, fM, yk, arN, AO, V, pd, q, er, Zp, akG, aOY, cPE, hW, nX, afQ, qK, aT, rO, rD, Nc, Tj, aPa, avt, aOV, ae, le, xR, Y, Lf, afS, r, en, bu, t, Ud, iK, sS, QG, kP, cPA, zu, KY, rA, id, atS, jh, Hn, zu, adg, cPC, ad, aOZ, av, hd, ib, Db, lQ, wm, fb, nJ, aeQ, ff, eH, cr, eZ, asV, Ej, bz, atb, dr, LB, z, afT, y, xg, iu); #c1(TP63) c2(NP_001108454) c3(tumor protein 63 isoform 6) c4(43249) c5(43250, 43251, 43252, 43253, 43254, 43255) c6( ) c7(aOR, bM, T, aC, Fv, if, ci, cPD, YD, ip, Na, aOS, aE, arA, pX, Eo, dV, K, hu, eL, Ur, li, iE, aW, cPB, are, az, Az, aOW, OZ, aEI, LZ, ark, g, eE, ZE, eF, cm, eM, Jr, cPc, lq, nB, vA, nF, wK, cj, aPc, k, m, cE, wu, iB, zt, ht, Bm, n, qN, ld, aPb, BI, ft, amJ, PD, cU, h, dL, Dz, aOU, aOX, A, MD, ks, fe, po, aOT, op, Ye, Qy, Qu, eD, hR, at, eo, YH, it, B, lp, fM, yk, arN, AO, V, pd, q, er, Zp, akG, aOY, cPE, hW, nX, afQ, qK, aT, rO, rD, Nc, Tj, aPa, avt, aOV, ae, le, xR, Y, Lf, afS, r, en, bu, t, Ud, iK, sS, QG, KP, cPA, zu, KY, rA, id, atS, jh, Hn, zU, adg, cPC, ad, aOZ, av, hd, ib, Db, lQ, wm, fb, nJ, aed, ff, eH, cr, eZ, asV, Ej, bz, atb, dr, LB, z, afT, y, xg, iu); #c1(TP63) c2(NP_003713) c3 (tumor protein 63 isoform 1) c4(140367, 140368) c5(140369, 140370, 140371, 140372, 140373, 140374, 140375) c6( ) c7(aOR, bM, T, aC, Fv, if, ci, cPD, YD, ip, Na, aOS, aE, arA, pX, Eo, dV, K, hu, eL, Ur, li, iE, aW, cPB, are, az, Az, aOW, OZ, aEI, LZ, ark, g, eE, ZE, eF, cm, eM, Jr, cPc, lq, nB, vA, nF, wK, cj, aPc, k, m, cE, wu, iB, zt, ht, Bm, n, qN, ld, aPb, BI, ft, amJ, PD, cU, h, dL, Dz, aOU, aOX, A, MD, ks, fe, po, aOT, op, Ye, Qy, Qu, eD, hR, at, eo, YH, it, B, lp, fM, yk, arN, AO, V, pd, q, er, Zp, akG, aOY, cPE, hW, nX, afQ, qK, aT, rO, rD, Nc, Tj, aPa, avt, aOV, ae, le, xR, Y, Lf, afS, r, en, bu, t, Ud, iK, sS, QG, kP, cPA, zu, KY, rA, id, atS, jh, Hn, zU, adg, cPC, ad, aOZ, av, hd, ib, Db, lQ, wm, fb, nJ, aeQ, ff, eH, cr, eZ, asV, Ej, bz, atb, dr, LB, z, afT, y, xg, iu); #c1(TP63) c2(XP_005247900) c3(tumor protein 63 isoform X3) c4(268033, 268034) c5(268035, 268036, 268037, 268038, 268039, 268040) c6( ) c7(aOR, bM, T, aC, Fv, if, ci, cPD, YD, ip, Na, aOS, aE, arA, pX, Eo, dV, K, hu, eL, Ur, li, iE, aW, cPB, are, az, Az, aOW, OZ, aEI, LZ, arK, g, eE, zE, eF, cm, eM, Jr, cPc, lq, nB, vA, nF, wK, cj, aPc, k, m, cE, wu, iB, zt, ht, Bm, n, qN, ld, aPb, BI, ft, amJ, PD, cU, h, dL, Dz, aOU, aOX, A, MD, ks, fe, po, aOT, op, Ye, Qy, Qu, eD, hR, at, eo, YH, it, B, lp, fM, yk, arN, AD, V, pd, q, er, Zp, akG, aOY, cPE, hW, nX, afQ, qK, aT, rO, rD, Nc, Tj, aPa, avt, aOV, ae, le, xR, Y, Lf, afS, r, en, bu, t, Ud, iK, sS, QG, kP, cPA, zu, KY, rA, id, atS, jh, Hn, zu, adg, cPC, ad, aOZ, av, hd, ib, Db, lQ, wm, fb, nJ, aeQ, ff, eH, cr, eZ, asV, Ej, bz, atb, dr, LB, z, afT, y, xg, iu); #c1(TP63) c2(XP_005247901) c3(tumor protein 63 isoform X5) c4( ) c5(268041, 268042, 268043, 268044, 268045, 268046, 268047, 268048, 268049, 268050, 268051, 268052) c6( ) c7(aOR, bM, T, aC, Fv, if, ci, cPD, YD, ip, Na, aOS, aE, arA, pX, Eo, dV, K, hu, eL, Ur, li, iE, aW, cPB, are, az, Az, aOW, OZ, aEI, LZ, ark, g, eE, ZE, eF, cm, eM, Jr, cPc, lq, nB, vA, nF, wK, cj, aPc, k, m, cE, wu, iB, zt, ht, Bm, n, qN, ld, aPb, BI, ft, amJ, PD, cU, h, dL, Dz, aOU, aOX, A, MD, ks, fe, po, aOT, op, Ye, Qy, Qu, eD, hR, at, eo, YH, it, B, lp, fM, yk, arN, AO, V, pd, q, er, Zp, akG, aOY, cPE, hW, nX, afQ, qK, aT, rO, rD, Nc, Tj, aPa, avt, aOV, ae, le, xR, Y, Lf, afS, r, en, bu, t, Ud, iK, sS, QG, kP, cPA, zu, KY, rA, id, atS, jh, Hn, zU, adg, cPC, ad, aOZ, av, hd, ib, Db, lQ, wm, fb, nJ, aeQ, ff, eH, cr, eZ, asV, Ej, bz, atb, dr, LB, z, afT, y, xg, iu); #c1(TP73) c2(XP_011540366) c3(tumor protein p73 isoform X1) c4(338854, 338855) c5(338856, 338857, 338858, 338859, 338860, 338861, 338862, 338863, 338864, 338865, 338866, 338867, 338868) c6( ) c7(gh, T, os, aC, fR, ea, if, al, fd, bB, ci, ip, x, aE, Ma, K, hu, eL, Jl, aW, g, as, eF, ir, cm, bw, eM, nB, iA, nF, iT, cj, iS, k, m, Xu, dD, ht, NT, n, afE, fr, qN, br, co, jz, hn, dL, hB, MD, GA, aq, Og, Zj, YE, bP, dB, eD, hL, JM, pV, eo, it, B, fM, jx, aA, hy, V, q, eA, qK, aT, j, ae, af, jf, qw, s, iI, Y, r, en, hf, cl, t, iK, ck, cT, ac, gX, aor, bt, aD, iW, ha, jh, oZ, oH, eq, hF, Fc, ad, afb, hX, iC, av, hd, NS, io, yF, d, BN, afF, ff, eH, Of, aF, bz, Hp, es, gk, aBU, bE, aS, adg, iu, BA); #c1(TPBG) c2(XP_011534399) c3 (trophoblast glycoprotein isoform X1) c4(330821) c5(330822, 330823, 330824, 330825, 330826, 330827, 330828, 330829, 330830, 330831, 330832, 330833, 330834, 330835) c6( ) c7(T, j, ci, fb, uC, fq, cd, ad); #c1(TPCN1) c2(NP_001137291) c3(two pore calcium channel protein 1 isoform 1) c4(55291, 55292) c5(55293, 55294, 55295, 55296, 55297, 55298, 55299, 55300, 55301, 55302, 55303) c6( ) c7(hd, nM, cc, Db, nB, ci, cd); #c1(TPCN1) c2(NP_001288143) c3(two pore calcium channel protein 1 isoform 3) c4( ) c5(113611, 113612, 113613, 113614, 113615) c6( ) c7(hd, nM, cc, Db, nB, ci, cd); #c1(TPCN1) c2(NP_060371) c3(two pore calcium channel protein 1 isoform 2) c4(195654, 195655, 195656) c5(195657, 195658, 195659, 195660, 195661, 195662, 195663, 195664, 195665, 195666, 195667, 195668, 195669, 195670, 195671, 195672) c6( ) c7(hd, nM, cc, Db, nB, ci, cd); #c1(TPCN1) c2(XP_005253962) c3 (two pore calcium channel protein 1 isoform X5) c4( ) c5(272790, 272791) c6( ) c7(hd, nM, cc, Db, nB, ci, cd); #c1(TPCN2) c2(NP_620714) c3(two pore calcium channel protein 2) c4(238111) c5(238112, 238113, 238114, 238115, 238116, 238117) c6( ) c7(cPF, Sc, cc, cd, ad); #c1(TPCN2) c2(XP_006718519) c3(two pore calcium channel protein 2 isoform X14) c4( ) c5(291888, 291889, 291890, 291891, 291892, 291893) c6( ) c7(cPF, Sc, cc, cd, ad); #c1(TPCN2) c2(XP_011543107) c3(two pore calcium channel protein 2 isoform X8) c4(341956) c5( ) c6( ) c7(cPF, Sc, cc, cd, ad); #c1(TPD52) c2(NP_001020423) c3(tumor protein D52 isoform 1) c4(28787, 28788, 28789) c5(28790, 28791, 28792, 28793, 28794, 28795, 28796, 28797, 28798, 28799, 28800, 28801, 28802, 28803, 28804, 28805, 28806) c6( ) c7(bL, T, eR, wu, aw, aj, v, ci, J, ad, Bj, aT, wm, ae, bA, s, aW, Y, t, ou, eD, m, ei, y, X); #c1(TPD52) c2(NP_001020424) c3(tumor protein D52 isoform 2) c4(28807) c5(28808, 28809, 28810, 28811, 28812, 28813) c6( ) c7(bL, T, eR, wu, aw, aj, v, ci, J, ad, Bj, aT, wm, ae, bA, s, aW, Y, t, ou, eD, m, ei, y, X); #c1(TPD52) c2(NP_001274069) c3(tumor protein D52 isoform 4) c4(106006) c5(106007) c6( ) c7(bL, T, eR, wu, aw, aj, v, ci, J, ad, Bj, aT, wm, ae, bA, s, aW, Y, t, ou, eD, m, ei, y, X); #c1(TPD52) c2(NP_001274071) c3(tumor protein D52 isoform 5) c4(106008) c5(106009, 106010, 106011, 106012, 106013, 106014) c6( ) c7(bL, T, eR, wu, aw, aj, v, ci, J, ad, Bj, aT, wm, ae, bA, s, aW, Y, t, ou, eD, m, ei, y, X); #c1(TPD52) c2(NP_005070) c3(tumor protein D52 isoform 3) c4(152595) c5(152596, 152597, 152598, 152599, 152600, 152601, 152602, 152603, 152604, 152605, 152606, 152607) c6( ) c7(bL, T, eR, wu, aw, aj, v, ci, J, ad, Bj, aT, wm, ae, bA, s, aW, Y, t, ou, eD, m, ei, y, X); #c1(TPD52L1) c2(NP_001003396) c3(tumor protein D53 isoform 3) c4(19797) c5(19798, 19799, 19800, 19801, 19802, 19803) c6( ) c7(aW, iV, fq, ad, aT); #c1(TPD52L1) c2(NP_001003397) c3(tumor protein D53 isoform 4) c4(19804, 19805) c5(19806, 19807, 19808, 19809, 19810, 19811, 19812, 19813, 19814, 19815) c6( ) c7(aW, iV, fq, ad, aT); #c1(TPD52L1) c2(NP_003278) c3(tumor protein D53 isoform 1) c4(137043, 137044, 137045, 137046) c5(137047, 137048, 137049, 137050, 137051, 137052, 137053, 137054, 137055, 137056, 137057, 137058, 137059) c6( ) c7(aW, iV, fq, ad, aT); #c1(TPD52L1) c2(XP_005267179) c3(tumor protein D53 isoform X1) c4( ) c5(280817, 280818, 280819, 280820, 280821) c6( ) c7(aW, iV, fq, ad, aT); #c1(TPD52L1) c2(XP_011534400) c3(tumor protein D53 isoform X2) c4( ) c5(330836, 330837, 330838, 330839, 330840, 330841, 330842, 330843, 330844, 330845, 330846, 330847, 330848, 330849, 330850, 330851) c6( ) c7(aW, iV, fq, ad, aT); #c1(TPD52L2) c2(NP_001230821) c3(tumor protein D54 isoform h) c4(86775) c5(86776, 86777, 86778, 86779, 86780, 86781) c6( ) c7(T, eA); #c1(TPD52L2) c2(NP_003279) c3(tumor protein D54 isoform e) c4(137060) c5(137061, 137062, 137063, 137064, 137065, 137066) c6( ) c7(T, eA); #c1(TPD52L2) c2(NP_955391) c3(tumor protein D54 isoform f) c4(261667) c5(261668, 261669, 261670, 261671, 261672, 261673) c6( ) c7(T, eA); #c1(TPD52L2) c2(NP_955392) c3(tumor protein 054 isoform a) c4(261674) c5(261675, 261676, 261677, 261678, 261679, 261680) c6( ) c7(T, eA); #c1(TPD52L2) c2(NP_955393) c3(tumor protein D54 isoform b) c4(261681) c5(261682, 261683, 261684, 261685, 261686, 261687) c6( ) c7(T, eA); #c1(TPD52L2) c2(NP_955394) c3(tumor protein D54 isoform c) c4(261688) c5(261689, 261690, 261691, 261692, 261693, 261694) c6( ) c7(T, eA); #c1(TPD52L2) c2(NP_955395) c3(tumor protein D54 isoform d) c4(261695) c5(261696, 261697, 261698, 261699, 261700, 261701) c6( ) c7(T, eA); #c1(TPGS2) c2(NP_001258879) c3(tubulin polyglutamylase complex subunit 2 isoform 3) c4(96647) c5(96648, 96649, 96650, 96651, 96652, 96653) c6( ) c7(aj, A, J); #c1(TPGS2) c2(NP_056291) c3(tubulin polyglutamylase complex subunit 2 isoform 1) c4(186951) c5(186952, 186953, 186954, 186955, 186956, 186957) c6( ) c7(aj, A, J); #c1(TPH1) c2(NP_004170) c3(tryptophan 5-hydroxylase 1) c4(144037, 144038) c5(144039, 144040, 144041, 144042, 144043, 144044, 144045, 144046, 144047, 144048, 144049, 144050) c6( ) c7(ID, tJ, FW, bok, FG, ago, al, bO, cz, bS, gf, ct, uu, v, ww, qi, aR, jz, zw, iX, pX, aql, MU, bma, bK, K, mY, he, rz, af, na, bA, wC, ly, tb, Gv, HL, aTY, bQ, Z, akb, lx, hq, bN, CA, ZT, Gs, mg, bLi, vL, Ei, uJ, FI, aTU, pw, ra, lC, jd, cPG); #c1(TPH2) c2(NP_775489) c3(tryptophan 5-hydroxylase 2) c4(250070, 250071) c5(250072, 250073, 250074, 250075, 250076, 250077, 250078, 250079, 250080, 250081, 250082, 250083) c6( ) c7(tJ, FW, al, bO, mS, Gd, bS, gf, ct, To, aqu, bMs, uu, qi, ww, aQF, rc, wy, W, ft, w, mW, iX, aVM, aql, bma, aaw, bK, K, aqD, oi, mY, mV, he, bME, rz, na, xf, rE, mU, wC, aWm, cPH, tb, yL, xs, dq, EZ, Z, vo, cM, mZ, bN, mX, hq, pH, cA, Ei, uJ, ei, ra, pk, lf, xg, mT, agZ, qc); #c1(TPI1) c2(NP_000356) c3(triosephosphate isomerase isoform 1) c4(7832) c5(7833, 7834, 7835, 7836, 7837, 7838) c6( ) c7(bM, cPI, T, aA, cHG, HH, V, eA, aR, PW, xl, aWJ, aT, Ak, dV, af, bA, iU, Y, ff, eZ, tv, dq, dg, tu, S, en, g, af, zz, dr, iP, cT, mO, dc, cPJ, byG, CI, lM, it, B, afX); #c1(TPI1) c2(NP_001152759) c3(triosephosphate isomerase isoform 2) c4(60164, 60165) c5(60166, 60167, 60168, 60169, 60170, 60171, 60/72) c6( ) c7(bM, cPI, T, aA, cHG, HH, V, eA, aR, PW, xl, aWJ, aT, Ak, dV, af, bA, iU, Y, ff, eZ, tv, dq, dg, tu, S, en, g, af, zz, dr, iP, cT, mO, dc, cPJ, byG, CI, lM, it, B, afX); #c1(TPI1) c2(NP_001244955) c3 (triosephosphate isomerase isoform 3) c4( ) c5(92436, 92437, 92438, 92439, 92440, 92441, 92442, 92443) c6( ) c7(bM, cPI, T, aA, cHG, HH, V, eA, aR, PW, xl, aWJ, aT, Ak, dV, af, bA, iU, Y, ff, eZ, tv, dq, dg, tu, S, en, g, aF, zz, dr, iP, cT, mO, dc, cPJ, byG, CI, lM, it, B, afX); #c1(TPK1) c2(NP_001035947) c3(thiamin pyrophosphokinase 1 isoform b) c4(33508) c5(33509, 33510, 33511, 33512, 33513, 33514) c6( ) c7(bL, oe, cPK, ac, an); #c1(TPK1) c2(XP_005250027) c3(thiamin pyrophosphokinase 1 isoform X1) c4(270027, 270028) c5(270029, 270030, 270031, 270032, 270033, 270034, 270035, 270036, 270037, 270038, 270039, 270040) c6( ) c7(bL, oe, cPK, ac, an); #c1(TPK1) c2(XP_011514337) c3(thiamin pyrophosphokinase 1 isoform X3) c4(307049) c5(307050, 307051, 307052, 307053, 307054, 307055) c6( ) c7(bL, oe, cPK, ac, an); #c1(TPM1) c2(NP_000357) c3(tropomyosin alpha-1 chain isoform Tpm1.5cy) c4(7839) c5(7840, 7841, 7842, 7843, 7844, 7845) c6( ) c7(Mb, cPM, T, Am, ht, cz, akA, ci, q, cd, eA, jz, cc, Ag, oj, aT, vc, aWs, K, anR, cPL, aW, Y, r, dg, mo, dr, bfW, cl, Ai, mx, afW, ei, it, ao); #c1(TPM1) c2(NP_001018004) c3(tropomyosin alpha-1 chain isoform Tpm1.6cy) c4(26760) c5(26761, 26762, 26763, 26764, 26765, 26766) c6( ) c7(Mb, cPM, T, Am, ht, cz, akA, ci, q, cd, eA, jz, cc, Ag, oj, aT, vc, aWs, K, anR, cPL, aW, Y, r, dg, mo, dr, bfW, cl, Ai, mx, afW, ei, it, ao); #c1(TPM1) c2(NP_001018005) c3(tropomyosin alpha-1 chain isoform Tpm1.1st) c4(26767, 26768) c5(26769, 26770, 26771, 26772, 26773, 26774, 26775, 26776, 26777, 26778, 26779, 26780) c6( ) c7(Mb, cPM, T, Am, ht, cz, akA, ci, q, cd, eA, jz, cc, Ag, oj, aT, vc, aWs, K, anR, cPL, aW, Y, r, dg, mo, dr, bfW, cl, Ai, mx, afW, ei, it, ao); #c1(TPM1) c2(NP_001018006) c3 (tropomyosin alpha-1 chain isoform Tpm1.7cy) c4(26781) c5(26782, 26783, 26784, 26785, 26786, 26787) c6( ) c7(Mb, cPM, T, Am, ht, cz, akA, ci, q, cd, eA, jz, cc, Ag, oj, aT, vc, aWs, K, anR, cPL, aW, Y, r, dg, mo, dr, bfW, cl, Ai, mx, afW, ei, it, ao); #c1(TPM1) c2(NP_001018007) c3(tropomyosin alpha-1 chain isoform Tpm1.4sm) c4(26788) c5(26789, 26790, 26791, 26792, 26793, 26794) c6( ) c7(Mb, cPM, T, Am, ht, cz, akA, ci, q, cd, eA, jz, cc, Ag, oj, aT, vc, aWs, K, anR, cPL, aW, Y, r, dg, mo, dr, bfW, cl, Ai, mx, afW, ei, it, ao); #c1(TPM1) c2(NP_001018008) c3(tropomyosin alpha-1 chain isoform Tpm1.12br) c4(26795) c5(26796, 26797, 26798, 26799, 26800, 26801) c6( ) c7(Mb, cPM, T, Am, ht, cz, akA, ci, q, cd, eA, jz, cc, Ag, oj, aT, vc, aWs, K, anR, cPL, aW, Y, r, dg, mo, dr, bfW, cl, Ai, mx, afW, ei, it, ao); #c1(TPM1) c2(NP_001018020) c3 (tropomyosin alpha-1 chain isoform Tpm1.3sm) c4(26866) c5(26867, 26868, 26869, 26870, 26871, 26872) c6( ) c7(Mb, cPM, T, Am, ht, cz, akA, ci, q, cd, eA, jz, cc, Ag, oj, aT, vc, aWs, K, anR, cPL, aW, Y, r, dg, mo, dr, bfW, cl, Ai, mx, afW, ei, it, ao); #c1(TPM1) c2(XP_005254694) c3(tropomyosin alpha-1 chain isoform X15) c4(273297) c5(273298, 273299, 273300, 273301, 273302, 273303) c6( ) c7(Mb, cPM, T, Am, ht, cz, akA, ci, q, cd, eA, jz, cc, Ag, oj, aT, vc, aWs, K, anR, cPL, aW, Y, r, dg, mo, dr, bfW, cl, Ai, mx, afW, ei, it, ao); #c1(TPM2) c2(NP_001288155) c3 (tropomyosin beta chain isoform Tpm2.3) c4(113616, 113617) c5(113618, 113619, 113620, 113621, 113622, 113623, 113624, 113625, 113626, 113627, 113628, 113629) c6( ) c7(chE, XW, cPN, chF, are, Am, Af, Ad, oj, it, vc, chy, bl, bBe, ae, cPO, ad); #c1(TPM2) c2(NP_003280) c3(tropomyosin beta chain isoform Tpm2.2st) c4(137067) c5(137068, 137069, 137070, 137071, 137072, 137073) c6( ) c7(chE, XW, cPN, chF, are, Am, Af, Ad, oj, it, vc, chy, bl, bBe, ae, cPO, ad); #c1(TPM2) c2(NP_998839) c3(tropomyosin beta chain isoform Tpm2.1sm/cy) c4(266064) c5(266065, 266066, 266067, 266068, 266069, 266070) c6( ) c7(chE, XW, cPN, chF, are, Am, Af, Ad, oj, it, vc, chy, bl, bBe, ae, cPO, ad); #c1(TPM3) c2(NP_001036816) c3(tropomyosin alpha-3 chain isoform Tpm3.2cy) c4(34094) c5(34095, 34096, 34097, 34098, 34099, 34100) c6( ) c7(T, cPP, if, Am, iW, jh, aA, n, ci, qN, zP, eA, hd, no, Ag, TK, oj, vc, Ou, K, nJ, jf, iF, zQ, xX, d, B, Af, Ad, aq, g, as, iR, cPQ, cm, iV, Ta, xA, nM, bP, nB, Ai, xj, Km, X, Tc); #c1(TPM3) c2(NP_001036817) c3(tropomyosin alpha-3 chain isoform Tpm3.7cy) c4(34101) c5(34102, 34103, 34104, 34105, 34106, 34107) c6( ) c7(T, cPP, if, Am, iW, jh, aA, n, ci, qN, zP, eA, hd, no, Ag, TK, oj, vc, Ou, K, nJ, jf, iF, zl, xX, d, B, Af, Ad, aq, g, as, iR, cPQ, cm, iV, Ta, xA, nM, bP, nB, Ai, xj, Km, X, Tc); #c1(TPM3) c2(NP_001036818) c3(tropomyosin alpha-3 chain isoform Tpm3.4cy) c4(34108) c5(34109, 34110, 34/11, 34112, 34113, 34114) c6( ) c7(T, cPP, if, Am, iW, jh, aA, n, ci, qN, zP, eA, hd, no, Ag, TK, oj, vc, Ou, K, nJ, jf, iF, zl, xX, d, B, Af, Ad, aq, g, as, iR, cPQ, cm, iV, Ta, xA, nM, bP, nB, Ai, xj, Km, X, Tc); #c1(TPM3) c2(NP_001265118) c3(tropomyosin alpha-3 chain isoform Tpm3.5cy) c4( ) c5(97809, 97810, 97811, 97812, 97813, 97814) c6( ) c7(T, cPP, if, Am, iW, jh, aA, n, ci, qN, zP, eA, hd, no, Ag, TK, oj, vc, Ou, K, nJ, jf, iF, zl, xX, d, B, Af, Ad, aq, g, as, iR, cPQ, cm, iV, Ta, xA, nM, bP, nB, Ai, xj, Km, X, Tc); #c1(TPM3) c2(NP_689476) c3(tropomyosin alpha-3 chain isoform Tpm3.12st) c4( ) c5(242818, 242819, 242820, 242821, 242822, 242823) c6( ) c7(T, cPP, if, Am, iW, jh, aA, n, ci, qN, zP, eA, hd, no, Ag, TK, oj, vc, Ou, K, nJ, jf, iF, zl, xX, d, B, Af, Ad, aq, g, as, iR, cPQ, cm, iV, Ta, xA, nM, bP, nB, Ai, xj, Km, X, Tc); #c1(TPM3) c2(NP_705935) c3(tropomyosin alpha-3 chain isoform Tpm3.1cy) c4(246458, 246459) c5(246460, 246461, 246462, 246463, 246464, 246465, 246466, 246467, 246468, 246469, 246470, 246471) c6( ) c7(T, cPP, if, Am, iW, jh, aA, n, ci, qN, zP, eA, hd, no, Ag, TK, oj, vc, Ou, K, nJ, jf, if, zQ, xX, d, B, Af, Ad, aq, g, as, iR, cPQ, cm, iV, Ta, xA, nM, bP, nB, Ai, xj, Km, X, Tc); #c1(TPM3) c2(XP_006711578) c3(tropomyosin alpha-3 chain isoform X3) c4(286152) c5(286153, 286154, 286155, 286156, 286157, 286158) c6( ) c7(T, cPP, if, Am, iW, jh, aA, n, ci, qN, zP, eA, hd, no, Ag, TK, oj, vc, Ou, K, nJ, jf, iF, zl, xX, d, B, Af, Ad, aq, g, as, iR, cPQ, cm, iV, Ta, xA, nM, bP, nB, Ai, xj, Km, X, Tc); #c1(TPM3) c2(XP_006711584) c3(tropomyosin alpha-3 chain isoform X8) c4( ) c5(286159, 286160, 286161, 286162, 286163, 286164) c6( ) c7(T, cPP, if, Am, iW, jh, aA, n, ci, qN, zP, eA, hd, no, Ag, TK, oj, vc, Ou, K, nJ, jf, iF, zl, xX, d, B, Af, Ad, aq, g, as, iR, cPQ, cm, iV, Ta, xA, nM, bP, nB, Ai, xj, Km, X, Tc); #c1(TPM3) c2(XP_011508253) c3(tropomyosin alpha-3 chain isoform X2) c4(298948, 298949) c5( ) c6( ) c7(T, cPP, if, Am, iW, jh, aA, n, ci, qN, zP, eA, hd, no, Ag, TK, oj, vc, Ou, K, nJ, jf, iF, z, xX, d, B, Af, Ad, aq, g, as, iR, cPQ, cm, iV, Ta, xA, nM, bP, nB, Ai, xj, Km, X, Tc); #c1(TPM3) c2(XP_011508254) c3 (tropomyosin alpha-3 chain isoform X9) c4(298950) c5( ) c6( ) c7(T, cPP, if, Am, iW, jh, aA, n, ci, qN, zP, eA, hd, no, Ag, TK, oj, vc, Ou, K, nJ, jf, iF, zl, xX, d, B, Af, Ad, aq, g, as, iR, cPQ, cm, iV, Ta, xA, nM, bP, nB, Ai, xj, Km, X, Tc); #c1(TPM4) c2(NP_001138632) c3(tropomyosin alpha-4 chain isoform Tpm4.1cy) c4(57292) c5(57293, 57294, 57295, 57296, 57297, 57298, 57299, 57300, 57301, 57302) c6( ) c7(qt, ht, bA); #c1(TPM4) c2(NP_003281) c3(tropomyosin alpha-4 chain isoform Tpm4.2cy) c4(137074) c5(137075, 137076, 137077, 137078, 137079, 137080) c6( ) c7(qt, ht, bA); #c1(TPMT) c2(NP_000358) c3(thiopurine S-methyltransferase) c4(7846) c5(7847, 7848, 7849, 7850, 7851, 7852) c6( ) c7(bL, gV, T, Am, aTP, oY, bh, oL, hF, V, ai, x, fX, fS, aT, h, hn, lQ, cPR, bc, ln, cPT, BN, aix, hG, hl, eZ, ge, r, fe, iV, cM, ck, cPS, ou, hA, dB, bE, aiS, XO, k, fL, gg, y, bt); #c1(TPO) c2(NP_001193673) c3(thyroid peroxidase isoform a precursor) c4(82459) c5(82460, 82461, 82462, 82463, 82464, 82465, 82466, 82467, 82468, 82469, 82470) c6( ) c7(T, os, aCh, fR, al, cLo, ci, aon, Vp, xy, Sq, K, biP, dF, hl, kt, bw, eM, nB, dD, aw, yG, aj, RN, cPV, dL, DU, A, bxz, Hz, aq, cA, pg, fL, Po, B, cGr, bL, AS, jx, fZ, GC, hz, bX, Wa, biO, q, J, eA, mP, nv, Fx, bK, hK, ae, ak, s, iU, r, t, ck, nM, hr, KA, gT, cJY, oZ, pl, eq, aYs, mC, ad, hd, F, Db, cLq, bpp, cMf, hD, yF, lx, wc, eZ, kD, xv, hp, bz, dr, gk, Lj, cPU, ei); #c1(TPO) c2(NP_001193674) c3(thyroid peroxidase isoform b precursor) c4(82471) c5(82472, 82473, 82474, 82475, 82476, 82477) c6( ) c7(T, os, aCh, fR, al, cLo, ci, aon, Vp, xy, Sq, K, biP, dF, hl, kt, bw, eM, nB, dD, aw, yG, aj, RN, cPV, dL, DU, A, bxz, Hz, aq, cA, pg, fL, Po, B, cGr, bL, AS, jx, fZ, GC, hz, bX, Wa, biO, q, J, eA, mP, nv, Fx, bK, hK, ae, ak, s, iU, r, t, ck, nM, hr, KA, gT, cJY, oZ, pl, eq, aYs, mC, ad, hd, F, Db, cLq, bpp, cMf, hD, yF, lx, wc, eZ, kD, xv, hp, bz, dr, gk, Lj, cPU, ei); #c1(TPO) c2(NP_783652) c3(thyroid peroxidase isoform d precursor) c4(251713) c5(251714, 251715, 251716, 25/717, 25/718, 251719) c6( ) c7(T, os, aCh, fR, al, cLo, ci, aon, Vp, xy, Sq, K, biP, dF, hl, kt, bw, eM, nB, dD, aw, yG, aj, RN, cPV, dL, DU, A, bxz, Hz, aq, cA, pg, fL, Po, B, cGr, bL, AS, jx, fZ, GC, hz, bX, Wa, biO, q, J, eA, mP, nv, Fx, bK, hK, ae, ak, s, iU, r, t, ck, nM, hr, KA, gT, cJY, oZ, pl, eq, aYs, mC, ad, hd, F, Db, cLq, bpp, cMf, hD, yF, lx, wc, eZ, kD, xv, hp, bz, dr, gk, Lj, cPU, ei); #c1(TPO) c2(NP_783653) c3(thyroid peroxidase isoform e precursor) c4(25/720) c5(25/721, 25/722, 251723, 251724, 25/725, 25/726, 25/727, 251728, 25/729, 251730) c6( ) c7(T, os, aCh, fR, al, cLo, ci, aon, Vp, xy, Sq, K, biP, dF, hl, kt, bw, eM, nB, dD, aw, yG, aj, RN, cPV, dL, DU, A, bxz, Hz, aq, cA, pg, fL, Po, B, cGr, bL, AS, jx, fZ, GC, hz, bX, Wa, biO, q, J, eA, mP, nv, Fx, bK, hK, ae, ak, s, iU, r, t, ck, nM, hr, KA, gT, cJY, oZ, pl, eq, aYs, mC, ad, hd, F, Db, cLq, bpp, cMf, hD, yF, lx, wc, eZ, kD, xv, hp, bz, dr, gk, Lj, cPU, ei); #c1(TPO) c2(XP_011508681) c3(thyroid peroxidase isoform X1) c4( ) c5(299290) c6( ) c7(T, os, aCh, fR, al, cLo, ci, aon, Vp, xy, Sq, K, biP, dF, hl, kt, bw, eM, nB, dD, aw, yG, aj, RN, cPV, dL, DU, A, bxz, Hz, aq, cA, pg, fL, Po, B, cGr, bL, AS, jx, fZ, GC, hz, bX, Wa, biO, q, J, eA, mP, nv, Fx, bK, hK, ae, ak, s, iU, r, t, ck, nM, hr, KA, gT, cJY, oZ, pl, eq, aYs, mC, ad, hd, F, Db, cLq, bpp, cMf, hD, yF, lx, wc, eZ, kD, xv, hp, bz, dr, gk, Lj, cPU, ei); #c1(TPO) c2(XP_011508682) c3(thyroid peroxidase isoform X2) c4(299291) c5(299292, 299293, 299294, 299295, 299296, 299297) c6( ) c7(T, os, aCh, fR, al, cLo, ci, aon, Vp, xy, Sq, K, biP, dF, hl, kt, bw, eM, nB, dD, aw, yG, aj, RN, cPV, dL, DU, A, bxz, Hz, aq, cA, pg, fL, Po, B, cGr, bL, AS, jx, fZ, GC, hz, bX, Wa, biO, q, J, eA, mP, nv, Fx, bK, hK, ae, ak, s, iU, r, t, ck, nM, hr, KA, gT, cJY, oZ, pl, eq, aYs, mC, ad, hd, F, Db, cLq, bpp, cMf, hD, yF, lx, wc, eZ, kD, xv, hp, bz, dr, gk, Lj, cPU, ei); #c1(TPO) c2(XP_01508684) c3(thyroid peroxidase isoform X4) c4(299298, 299299, 299300) c5(299301, 299302, 299303, 299304, 299305, 299306, 299307, 299308, 299309, 299310, 299311, 299312, 299313, 299314, 299315, 299316) c6( ) c7(T, os, aCh, fR, al, cLo, ci, aon, Vp, xy, Sq, K, biP, dF, hl, kt, bw, eM, nB, dD, aw, yG, aj, RN, cPV, dL, DU, A, bxz, Hz, aq, cA, pg, fL, Po, B, cGr, bL, AS, jx, fZ, GC, hz, bX, Wa, biO, q, J, eA, mP, nv, Fx, bK, hK, ae, ak, s, iU, r, t, ck, nM, hr, KA, gT, cJY, oZ, pl, eq, aYs, mC, ad, hd, F, Db, cLq, bpp, cMf, hD, yF, lx, wc, eZ, kD, xv, hp, bz, dr, gk, Lj, CPU, ei); #c1(TPP1) c2(NP_000382) c3(tripeptidyl-peptidase 1 preproprotein) c4(8336, 8337) c5(8338, 8339, 8340, 8341, 8342, 8343, 8344, 8345, 8346, 8347, 8348, 8349) c6( ) c7(eR, ha, ci, bX, ad, aR, auf, mP, hb, h, K, af, sb, d, iU, rU, dg, dr, rY, W, cPW, U, cj, Lq, aPM, bt, B); #c1(TPP2) c2(NP_003282) c3(tripeptidyl-peptidase 2) c4(137081) c5(137082, 137083, 137084, 137085, 137086, 137087) c6( ) c7(jh, U); #c1(TPPP) c2(NP_008961) c3(tubulin polymerization-promoting protein) c4(169931) c5(169932, 169933, 169934, 169935, 169936, 169937) c6( ) c7(if, iW, GC, fr, qN, aR, hX, ke, Hx, ds, jZ, dV, kH, h, SH, dL, an, he, eL, af, CB, ak, je, aHW, lo, iV, kk, ck, rR, iM, ac, c, cA, bE, NL, iQ, U, aEG, zv, aEN, gD, EB); #c1(TPPP2) c2(XP_005267381) c3(tubulin polymerization-promoting protein family member 2 isoform X1) c4(280918) c5(280919, 280920, 280921, 280922, 280923, 280924, 280925, 280926, 280927, 280928) c6( ) c7(ck, eM, Y, iS, fd, bw, v, K, hF, vt, B, ip); #c1(TPPP3) c2(NP_057224) c3(tubulin polymerization-promoting protein family member 3) c4(189563) c5(189564, 189565, 189566, 189567, 189568, 189569, 189570, 189571, 189572, 189573) c6( ) c7(cm, xs); #c1(TPR) c2(NP_003283) c3(nucleoprotein TPR) c4(137088, 137089) c5(137090, 137091, 137092, 137093, 137094, 137095, 137096, 137097, 137098, 137099, 137100, 137/01, 137102, 137103, 137104, 137105) c6( ) c7(T, os, d, nM, nB, Oy, awP, aA, oZ, aF, K, fu, bU, on); #c1(TPRG1) c2(NP_940887) c3(tumor protein p63-regulated gene 1 protein) c4( ) c5(260041, 260042, 260043, 260044, 260045, 260046, 260047, 260048) c6( ) c7(j, an); #c1(TPRG1) c2(XP_005247439) c3(tumor protein p63-regulated gene 1 protein isoform X2) c4(267834) c5(267835, 267836) c6( ) c7(j, an); #c1(TPRG1) c2(XP_005247445) c3(tumor protein p63-regulated gene 1 protein isoform X7) c4(267837) c5(267838, 267839, 267840, 267841, 267842, 267843) c6( ) c7(j, an); #c1(TPRN) c2(NP_001121700) c3(taperin) c4(46115, 46116) c5(46117, 46118, 46119, 46120, 46121, 46122, 46123, 46124, 46125, 46126, 46127, 46128) c6( ) c7(Bh, cPX); #c1(TPSAB1) c2(NP_003285) c3(tryptase alpha/beta-1 precursor) c4(137106, 137107, 137108) c5(137109, 137110, 137/11, 137112, 137113, 137114, 137115, 137116, 137117, 137118, 137119, 137120, 137121, 137122, 137123, 137124, 137125, 137126, 137127, 137128, 137129, 137130) c6( ) c7(bM, eG, m, y, V, Tc); #c1(TPSB2) c2(NP_077078) c3(tryptase beta-2 preproprotein) c4(214833) c5(214834, 214835, 214836, 214837, 214838, 214839) c6( ) c7(mQ, Tc); #c1(TPSD1) c2(NP_036349) c3(tryptase delta precursor) c4(173148, 173149) c5(173150, 173151, 173152, 173153, 173154, 173155, 173156, 173157, 173158, 173159, 173160, 173161) c6( ) c7(Jm, bM, Ta, lU, HI, bm, fe, dy, lC, bt, Tc); #c1(TPSG1) c2(NP_036599) c3(tryptase gamma preproprotein) c4(174716) c5(174717, 174718, 174719, 174720, 174721, 174722) c6( ) c7(Jm, bM, Ta, BU, x, lU, HI, aW, bm, aT, at, fe, dy, aDR, k, ji, lC, bt, y, aDQ, Tc); #c1(TPSG1) c2(XP_011520749) c3(tryptase gamma isoform X1) c4( ) c5(315763) c6( ) c7(Jm, bM, Ta, BU, x, lU, HI, aW, bm, aT, at, fe, dy, aDR, k, ji, lC, bt, y, aDQ, Tc); #c1(TPT1) c2(NP_001273202) c3(translationally-controlled tumor protein isoform 3) c4( ) c5(104567, 104568, 104569, 104570, 104571, 104572, 104573, 104574, 104575, 104576, 104577, 104578, 104579) c6( ) c7(ID, bM, T, AS, cz, bB, alQ, ci, alP, alR, eA, ad, eJ, dP, av, aE, cfd, fS, dV, aT, aUm, K, ae, af, ly, eG, yi, aW, ev, Pg, lx, ck, qJ, Ew, bP, bE, U, auT, y, AN); #c1(TPT1) c2(NP_003286) c3(translationally-controlled tumor protein isoform 2) c4(137131, 137132, 137133, 137134) c5(137135, 137136, 137137, 137138, 137139, 137140, 137141, 137142, 137143, 137144, 137145, 137146, 137147, 137148, 137149, 137150, 137151, 137152, 137153, 137154, 137155, 137156, 137157, 137158, 137159) c6( ) c7(ID, bM, T, AS, cz, bB, aJI, ci, alP, alR, eA, ad, eJ, dP, av, aE, cfd, fS, dV, aT, aUm, K, ae, af, ly, eG, yi, aW, ev, Pg, lx, ck, qJ, Ew, bP, bE, U, auT, y, AN); #c1(TPTE) c2(NP_954868) c3 (putative tyrosine-protein phosphatase TPTE isoform beta) c4(261453) c5(261454, 261455, 261456, 261457, 261458, 261459) c6( ) c7(f); #c1(TPTE) c2(NP_954869) c3(putative tyrosine-protein phosphatase TPTE isoform gamma) c4(261460) c5(261461, 261462, 261463, 261464, 261465, 261466) c6( ) c7(f); #c1(TPTE) c2(NP_954870) c3(putative tyrosine-protein phosphatase TPTE isoform alpha) c4(261467) c5(261468, 261469, 261470, 261471, 261472, 261473) c6( ) c7(f); #c1(TPTE2) c2(NP_001135440) c3(phosphatidylinositol 3,4,5-trisphosphate 3-phosphatase TPTE2 isoform delta) c4(52891, 52892) c5(52893, 52894, 52895, 52896, 52897, 52898, 52899, 52900, 52901, 52902, 52903, 52904, 52905, 52906, 52907, 52908) c6( ) c7(aq, B, al); #c1(TPTE2) c2(NP_570141) c3(phosphatidylinositol 3,4,5-trisphosphate 3-phosphatase TPTE2 isoform alpha) c4(234065) c5(234066, 234067, 234068, 234069, 234070, 234071, 234072, 234073, 234074, 234075) c6( ) c7(aq, B, al); #c1(TPTE2) c2(NP_954863) c3(phosphatidylinositol 3,4,5-trisphosphate 3-phosphatase TPTE2 isoform gamma) c4(261428, 261429, 261430) c5(261431, 261432, 261433, 261434, 261435, 261436, 261437, 261438, 261439, 261440, 261441, 261442, 261443, 261444, 261445, 261446, 261447, 261448, 261449, 261450, 261451, 261452) c6( ) c7(aq, B, al); #c1(TPX2) c2(XP_011527001) c3(targeting protein for Xklp2 isoform X1) c4(321343, 321344) c5(321345, 321346, 321347, 321348, 321349, 321350, 321351, 321352, 321353, 321354, 321355, 321356) c6( ) c7(T, aC, if, iW, jh, fd, bB, n, ci, qN, q, ad, eJ, av, aE, kH, aT, ey, ae, je, qt, PX, ff, r, kp, en, as, cm, bz, iM, eM, bE, Qu, ahC, B, KG); #c1(TRA2A) c2(NP_001269688) c3(transformer-2 protein homolog alpha isoform 3) c4(102550) c5(102551, 102552, 102553, 102554, 102555, 102556, 102557, 102558, 102559, 102560, 102561, 102562, 102563, 102564, 102565, 102566, 102567, 102568) c6( ) c7(Pk, lQ); #c1(TRA2A) c2(NP_037425) c3(transformer-2 protein homolog alpha isoform 1) c4(175495, 175496) c5( ) c6( ) c7(Pk, lQ); #c1(TRA2A) c2(XP_011513633) c3(transformer-2 protein homolog alpha isoform X2) c4( ) c5(305943) c6( ) c7(Pk, lQ); #c1(TRA2B) c2(NP_001230808) c3(transformer-2 protein homolog beta isoform 2) c4( ) c5(86763, 86764, 86765, 86766, 86767, 86768, 86769, 86770, 86771, 86772, 86773, 86774) c6( ) c7(bM, Hx, W, bE, kf, bB, v, K, g, fq); #c1(TRA2B) c2(NP_004584) c3(transformer-2 protein homolog beta isoform 1) c4(147894, 147895) c5(147896, 147897, 147898, 147899, 147900, 147901, 147902) c6(147903) c7(bM, Hx, W, bE, kf, bB, v, K, g, fq); #c1(TRABD) c2(NP_079480) c3(traB domain-containing protein) c4( ) c5(218266, 218267, 218268, 218269, 218270, 218271, 218272, 218273, 218274, 218275, 218276) c6( ) c7(ck, x, aF, aA); #c1(TRABD) c2(XP_006724485) c3(traB domain-containing protein isoform X7) c4(295984) c5( ) c6( ) c7(ck, x, aF, aA); #c1(TRABD) c2(XP_011529016) c3(traB domain-containing protein isoform X4) c4( ) c5(324375, 324376, 324377) c6( ) c7(ck, x, aF, aA); #c1(TRABD2A) c2(NP_001074293) c3(metalloprotease TIKI1 isoform 2 precursor) c4(37075) c5(37076, 37077, 37078, 37079, 37080, 37081) c6( ) c7(ac); #c1(TRABD2A) c2(NP_001263982) c3(metalloprotease TIKI1 isoform 1 precursor) c4(97402) c5(97403, 97404, 97405, 97406, 97407, 97408) c6( ) c7(ac); #c1(TRADD) c2(XP_011521722) c3(tumor necrosis factor receptor type 1-associated DEATH domain protein isoform X1) c4(316438) c5(316439, 316440, 316441, 316442, 316443, 316444) c6( ) c7(Hw, d, mR, dB, iW, eH, eZ, aT, mK, oY, fd, eF, eA); #c1(TRAF1) c2(NP_001177876) c3(TNF receptor-associated factor 1 isoform b) c4(73116) c5(73117, 73118, 73119, 73120, 73121) c6( ) c7(Kd, T, dD, ea, iW, e, jh, bB, cL, eq, fr, C, EA, Uy, o, ad, ai, dP, eA, aT, h, ey, K, af, bc, aa, A, aW, axu, KS, ff, eZ, Au, en, g, pL, ck, bF, sS, OW, ac, zr, bE, go, cj, uJ, fD, y, Pr, X); #c1(TRAF1) c2(NP_005649) c3(TNF receptor-associated factor 1 isoform a) c4(158252) c5(158253, 158254, 158255, 158256, 158257, 158258, 158259, 158260, 158261, 158262, 158263) c6( ) c7(Kd, T, dD, ea, iW, e, jh, bB, cL, eq, fr, C, EA, Uy, o, ad, ai, dP, eA, aT, h, ey, K, af, bc, aa, A, aW, axu, KS, ff, eZ, Au, en, g, pL, ck, bF, sS, OW, ac, zr, bE, go, cj, uJ, fD, y, Pr, X); #c1(TRAF2) c2(NP_066961) c3(TNF receptor-associated factor 2) c4( ) c5(207798, 207799, 207800, 207801, 207802, 207803, 207804, 207805, 207806, 207807, 207808, 207809, 207810, 207811, 207812, 207813, 207814, 207815, 207816, 207817, 207818, 207819, 207820, 207821, 207822, 207823, 207824, 207825, 207826, 207827, 207828, 207829, 207830, 207831, 207832) c6( ) c7(bM, T, aC, fM, fR, sQ, iW, jh, aA, bB, fd, zh, qj, ze, fr, zo, ad, eA, ft, ib, aE, oW, aT, dV, h, K, zg, lQ, Od, ae, af, cp, eG, d, aW, Y, zb, ff, eZ, vt, aF, en, t, ve, bz, zr, bE, cj, uo, zl, ut, eg, zk, bt, iu, pC); #c1(TRAF2) c2(XP_005266156) c3(TNF receptor-associated factor 2 isoform X3) c4(279930, 279931, 279932, 279933) c5(279934, 279935, 279936, 279937, 279938, 279939, 279940, 279941, 279942, 279943, 279944) c6( ) c7(bM, T, aC, fM, fR, sQ, iW, jh, aA, bB, fd, zh, qj, ze, fr, zo, ad, eA, ft, ib, aE, oW, aT, dV, h, K, zg, lQ, Od, ae, af, cp, eG, d, aW, Y, zb, ff, eZ, vt, aF, en, t, ve, bz, zr, bE, cj, uo, zl, ut, eg, zk, bt, iu, pC); #c1(TRAF2) c2(XP_011517275) c3(TNF receptor-associated factor 2 isoform X2) c4(310866, 310867, 310868) c5(310869) c6( ) c7(bM, T, aC, fM, fR, s, iW, jh, aA, bB, fd, zh, qj, ze, fr, zo, ad, eA, ft, ib, aE, oW, aT, dV, h, K, zg, lQ, Od, ae, af, cp, eG, d, aW, Y, zb, ff, eZ, vt, af, en, t, ve, bz, zr, bE, cj, uo, zl, ut, eg, zk, bt, iu, pC); #c1(TRAF3) c2(NP_001186356) c3(TNF receptor-associated factor 3 isoform 3) c4(77507) c5(77508, 77509, 77510, 77511, 77512, 77513, 77514, 77515, 77516, 77517, 77518, 77519) c6( ) c7(T, iW, ex, jh, fd, aj, cPY, ci, q, J, ad, eW, dV, aT, al, h, eV, K, ae, af, aW, ff, xs, wc, aiT, lo, en, ck, bNF, cj, CZ); #c1(TRAF3) c2(NP_003291) c3(TNF receptor-associated factor 3 isoform 1) c4(137200) c5(137201, 137202, 137203, 137204, 137205, 137206, 137207, 137208, 137209, 137210) c6( ) c7(T, iW, eX, jh, fd, aj, cPY, ci, q, J, ad, eW, dV, aT, al, h, eV, K, ae, af, aW, ff, xs, wc, aiT, lo, en, ck, bNF, cj, CZ); #c1(TRAF3) c2(NP_663778) c3(TNF receptor-associated factor 3 isoform 2) c4(241544) c5(241545, 241546, 241547, 241548, 241549, 241550, 241551, 241552, 241553, 241554) c6( ) c7(T, iW, ex, jh, fd, aj, cPY, ci, q, J, ad, eW, dV, aT, aJI, h, eV, K, ae, af, aW, ff, xs, wc, aiT, lo, en, ck, bNF, cj, CZ); #c1(TRAF3) c2(XP_011535418) c3(TNF receptor-associated factor 3 isoform X2) c4(332461) c5( ) c6( ) c7(T, iW, eX, jh, fd, aj, cPY, ci, q, J, ad, eW, dV, aT, aJI, h, eV, K, ae, af, aW, ff, xs, wc, aiT, lo, en, ck, bNF, cj, CZ); #c1(TRAF3IP1) c2(NP_001132962) c3(TRAF3-interacting protein 1 isoform 2) c4(52720, 52721) c5(52722, 52723, 52724, 52725, 52726, 52727, 52728, 52729, 52730, 52731, 52732, 52733) c6( ) c7(bNF, afu); #c1(TRAF3IP1) c2(NP_056465) c3(TRAF3-interacting protein 1 isoform 1) c4(187631, 187632) c5(187633, 187634, 187635, 187636, 187637, 187638, 187639, 187640, 187641, 187642, 187643, 187644) c6( ) c7(bNF, afu); #c1(TRAF3IP2) c2(NP_001157753) c3(adapter protein CIKS isoform 3) c4(62639) c5(62640, 62641, 62642, 62643, 62644, 62645) c6( ) c7(dD, ea, fN, cR, aj, j, eq, Za, wD, cfd, aUm, K, cQa, hH, qY, HA, cr, eZ, dN, da, fe, iV, eE, Pa, Jr, wR, aS, ra, cPZ); #c1(TRAF3IP2) c2(NP_001157755) c3(adapter protein CIKS isoform 5) c4( ) c5(62646, 62647, 62648, 62649, 62650, 62651) c6( ) c7(dD, ea, fN, cR, aj, j, eq, Za, wD, cfd, aUm, K, cQa, hH, qY, HA, cr, eZ, dN, da, fe, iV, eE, Pa, Jr, wR, aS, ra, cPZ); #c1(TRAF3IP2) c2(NP_679211) c3(adapter protein CIKS isoform 2) c4(242188) c5(242189, 242190, 242191, 242192, 242193) c6( ) c7(dD, ea, fN, cR, aj, j, eq, Za, wD, cfd, aUm, K, cQa, hH, qY, HA, cr, eZ, dN, da, fe, iV, eE, Pa, Jr, wR, aS, ra, cPZ); #c1(TRAF4) c2(NP_004286) c3(TNF receptor-associated factor 4) c4(144847, 144848, 144849, 144850) c5(144851, 144852, 144853, 144854, 144855, 144856, 144857, 144858, 144859, 144860, 144861, 144862, 144863, 144864, 144865, 144866, 144867, 144868, 144869, 144870, 144871, 144872, 144873, 144874) c6( ) c7(T, aW, iK, aT, h, ci, K, g, aed, bz, af, TT); #c1(TRAF4) c2(XP_011523806) c3(TNF receptor-associated factor 4 isoform X1) c4(318328) c5(318329, 318330, 318331, 318332, 318333, 318334) c6( ) c7(T, aW, iK, aT, h, ci, K, g, aed, bz, af, TT); #c1(TRAF4) c2(XP_011523810) c3(TNF receptor-associated factor 4 isoform X4) c4(318335, 318336) c5(318337, 318338, 318339, 318340, 318341, 318342, 318343, 318344, 318345, 318346, 318347, 318348) c6( ) c7(T, aW, iK, aT, h, ci, K, g, aed, bz, af, TT); #c1(TRAF5) c2(NP_665702) c3(TNF receptor-associated factor 5) c4(241659) c5(241660, 241661, 241662, 241663, 241664, 241665, 241666, 241667, 241668, 241669, 241670, 241671, 241672, 241673, 241674, 241675, 241676, 241677, 241678, 241679) c6( ) c7(mR, iW, HA, jx, fd, h, lo, j, agl, hH, qW, af, y); #c1(TRAF5) c2(XP_011508259) c3(TNF receptor-associated factor 5 isoform X2) c4(298951) c5( ) c6( ) c7(mR, iW, HA, jx, fd, h, lo, j, agl, hH, qW, af, y); #c1(TRAF6) c2(NP_665802) c3(TNF receptor-associated factor 6) c4(241698) c5(241699, 24/700, 24/701, 241702, 241703, 24/704, 241705, 24/706, 24/707, 241708) c6( ) c7(oO, agx, T, dD, ea, iW, jx, jh, aA, it, fd, j, ci, C, agz, eq, mC, br, cF, bjA, bjv, aT, h, dL, bl, eL, af, bA, aW, Y, mQ, eZ, kD, kp, aF, bw, Zb, bz, pb, rR, lt, atM, Jr, nB, wR, cbL, fL, y, bt, Pr, iu); #c1(TRAF7) c2(NP_115647) c3(E3 ubiquitin-protein ligase TRAF7) c4(223422) c5(223423, 223424, 223425, 223426, 223427, 223428) c6( ) c7(bwj, ip); #c1(TRAFD1) c2(NP_006691) c3(TRAF-type zinc finger domain-containing protein 1) c4(167164, 167165, 167166, 167167) c5(167168, 167169, 167170, 167171, 167172, 167173, 167174, 167175, 167176, 167177, 167178, 167179, 167180, 167181, 167182, 167183, 167184, 167185, 167186, 167187, 167188, 167189, 167190, 167191, 167192, 167193, 167194, 167195) c6( ) c7(A); #c1(TRAK1) c2(NP_001036111) c3(trafficking kinesin-binding protein 1 isoform 1) c4(33984) c5(33985, 33986, 33987, 33988, 33989) c6( ) c7(IF, aR, NE, aA); #c1(TRAK1) c2(NP_001252539) c3(trafficking kinesin-binding protein 1 isoform 5) c4(94212) c5(94213, 94214, 94215, 94216, 94217, 94218) c6( ) c7(IF, aR, NE, aA); #c1(TRAK1) c2(NP_055780) c3(trafficking kinesin-binding protein 1 isoform 2) c4(183463) c5(183464, 183465, 183466, 183467, 183468, 183469) c6( ) c7(IF, aR, NE, aA); #c1(TRAK1) c2(XP_005265013) c3(trafficking kinesin-binding protein 1 isoform X2) c4(279330) c5(279331, 279332, 279333, 279334, 279335, 279336) c6( ) c7(IF, aR, NE, aA); #c1(TRAK1) c2(XP_005265014) c3(trafficking kinesin-binding protein 1 isoform X3) c4(279337) c5(279338, 279339, 279340, 279341, 279342, 279343) c6( ) c7(IF, aR, NE, aA); #c1(TRAK1) c2(XP_005265017) c3(trafficking kinesin-binding protein 1 isoform X6) c4( ) c5(279344) c6(279345) c7(IF, aR, NE, aA); #c1(TRAK1) c2(XP_011531789) c3(trafficking kinesin-binding protein 1 isoform X1) c4( ) c5(327880) c6( ) c7(IF, aR, NE, aA); #c1(TRAK2) c2(NP_055864) c3 (trafficking kinesin-binding protein 2) c4(183998, 183999, 184000) c5(184001, 184002, 184003, 184004, 184005, 184006, 184007, 184008, 184009, 184010, 184011, 184012, 184013, 184014, 184015, 184016, 184017) c6( ) c7(af, c); #c1(TRAK2) c2(XP_011509993) c3(trafficking kinesin-binding protein 2 isoform X3) c4(300611) c5(300612, 300613, 300614, 300615, 300616, 300617, 300618) c6( ) c7(af, c); #c1(TRAMI) c2(NP_055109) c3(translocating chain-associated membrane protein 1) c4(178996, 178997) c5(178998, 178999, 179000, 179001, 179002, 179003, 179004) c6( ) c7(eW, T, aW, ki, jx, aep, aT, hl, ey, Hb, ci, cl, g, gT, eA, ae, B, ad, afu); #c1(TRAM1L1) c2(NP_689615) c3(translocating chain-associated membrane protein 1-like 1) c4(243274) c5(243275, 243276, 243277, 243278, 243279, 243280) c6( ) c7(v, ad); #c1(TRAM2) c2(NP_036420) c3(translocating chain-associated membrane protein 2) c4(173679) c5(173680, 173681, 173682, 173683, 173684, 173685) c6( ) c7(aw); #c1(TRAP1) c2(NP_001258978) c3(heat shock protein 75 kDa, mitochondrial isoform 2) c4(96885) c5(96886, 96887, 96888, 96889, 96890, 96891) c6( ) c7(T, ac, tL, aW, bE, ht, aT, r, cy, aA, it, K, uE, hu, ae, q, ad); #c1(TRAP1) c2(NP_057376) c3(heat shock protein 75 kDa, mitochondrial isoform 1 precursor) c4(190462) c5(190463, 190464, 190465, 190466, 190467, 190468) c6( ) c7(T, ac, tL, aW, bE, ht, aT, r, cy, aA, it, K, uE, hu, ae, q, ad); #c1(TRAPPC1) c2(NP_067033) c3(trafficking protein particle complex subunit 1) c4(208539) c5(208540, 208541, 208542, 208543, 208544, 208545, 208546, 208547, 208548, 208549) c6( ) c7(F, jE); #c1(TRAPPC10) c2(NP_003265) c3(trafficking protein particle complex subunit 10) c4(136938) c5(136939, 136940, 136941, 136942, 136943, 136944) c6( ) c7(vj, ha, amj, yZ, lQ, nA, yU, Pk, axC); #c1(TRAPPC10) c2(XP_005261225) c3(trafficking protein particle complex subunit 10 isoform X4) c4(277270) c5(277271, 277272, 277273, 277274, 277275, 277276) c6( ) c7(vj, ha, amj, yZ, lQ, nA, yU, Pk, axC); #c1(TRAPPC10) c2(XP_011528019) c3(trafficking protein particle complex subunit 10 isoform X5) c4( ) c5(323118) c6( ) c7(vj, ha, amj, yZ, lQ, nA, yU, Pk, axC); #c1(TRAPPC11) c2(NP_068761) c3(trafficking protein particle complex subunit 11 isoform a) c4(210250, 210251) c5(210252, 210253, 210254, 210255, 210256, 210257) c6( ) c7(cQb, oj, km, aj, nA, xw, cd); #c1(TRAPPC11) c2(NP_951008) c3(trafficking protein particle complex subunit 11 isoform b) c4(260958) c5(260959, 260960, 260961, 260962, 260963, 260964) c6( ) c7(cQb, oj, km, aj, nA, xw, cd); #c1(TRAPPC11) c2(XP_011530482) c3(trafficking protein particle complex subunit 11 isoform X1) c4( ) c5(326378, 326379, 326380, 326381, 326382, 326383, 326384) c6( ) c7(cQb, oj, km, aj, nA, xw, cd); #c1(TRAPPC2) c2(NP_001122307) c3(trafficking protein particle complex subunit 2 isoform 2) c4(46472, 46473, 46474, 46475) c5(46476, 46477, 46478, 46479, 46480, 46481, 46482, 46483, 46484, 46485, 46486, 46487, 46488) c6( ) c7(ati, nA, Wr, QK, rv, dr, lc); #c1(TRAPPC2) c2(XP_011543868) c3(trafficking protein particle complex subunit 2 isoform X1) c4( ) c5(343000, 343001, 343002, 343003, 343004, 343005, 343006, 343007, 343008, 343009, 343010, 343011, 343012, 343013, 343014, 343015, 343016, 343017, 343018, 343019, 343020, 343021, 343022, 343023, 343024, 343025, 343026) c6( ) c7(ati, nA, Wr, QK, rv, dr, lc); #c1(TRAPPC4) c2(NP_057230) c3(trafficking protein particle complex subunit 4) c4(189606) c5(189607, 189608, 189609, 189610, 189611, 189612) c6( ) c7(q, an, s, r); #c1(TRAPPC9) c2(NP_001153844) c3 (trafficking protein particle complex subunit 9 isoform b) c4(61005) c5(61006, 61007, 61008, 61009, 61010) c6( ) c7(ac, dy, V, nA, QK, cQc, fq, cd, bN, lc); #c1(TRAPPC9) c2(NP_113654) c3(trafficking protein particle complex subunit 9 isoform a) c4(221956) c5(221957, 221958, 221959, 221960, 221961, 221962, 221963) c6( ) c7(ac, dy, V, nA, QK, cQc, fq, cd, bN, lc); #c1(TRAPPC9) c2(XP_011515628) c3(trafficking protein particle complex subunit 9 isoform X2) c4(308476) c5(308477, 308478, 308479, 308480, 308481, 308482) c6( ) c7(ac, dy, V, nA, QK, cQc, fq, cd, bN, lc); #c1(TRAPPC9) c2(XP_011515632) c3(trafficking protein particle complex subunit 9 isoform X6) c4( ) c5(308483) c6( ) c7(ac, dy, V, nA, QK, cQc, fq, cd, bN, lc); #c1(TRAT1) c2(NP_057472) c3(T-cell receptor-associated transmembrane adapter 1) c4(191129) c5(191130, 191131, 191132, 191133, 191134, 191135) c6( ) c7(T, aW, al, h, n, gn, bC, cm); #c1(TRDMT1) c2(NP_004403) c3(tRNA (cytosine(38)-C(5)-methyltransferase) c4(146133, 146134) c5(146135, 146136, 146137, 146138, 146139, 146140, 146141, 146142, 146143, 146144, 146145, 146146) c6( ) c7(T, ft, ac, eM, Ip, aA, m, bz, B, ad); #c1(TRDMT1) c2(XP_005252429) c3(tRNA (cytosine(38)-C(5)-methyltransferase isoform X2) c4(271788, 271789) c5( ) c6( ) c7(T, ft, ac, eM, lp, aA, m, bz, B, ad); #c1(TRDMT1) c2(XP_005252432) c3(tRNA (cytosine(38)-C(5)-methyltransferase isoform X5) c4(271790) c5(271791, 271792, 271793, 271794, 271795, 271796, 271797, 271798, 271799, 271800, 271801, 271802, 271803, 271804, 271805, 271806) c6( ) c7(T, ft, ac, eM, lp, aA, m, bz, B, ad); #c1(TRDMT1) c2(XP_011517647) c3(tRNA (cytosine(38)-C(5)-methyltransferase isoform X6) c4( ) c5(311491) c6( ) c7(T, ft, ac, eM, lp, aA, m, bz, B, ad); #c1(TRDMT1) c2(XP_011517649) c3(tRNA (cytosine(38)-C(5))-methyltransferase isoform X8) c4(311492) c5(311493, 311494, 311495, 311496, 311497, 311498, 311499) c6( ) c7(T, ft, ac, eM, lp, aA, m, bz, B, ad); #c1(TRDN) c2(NP_001238916) c3(triadin isoform 2) c4(88047) c5( ) c6( ) c7(cdd, cy, ey, NW, bK); #c1(TRDN) c2(NP_001242949) c3(triadin isoform 3) c4(89371, 89372) c5( ) c6( ) c7(cdd, cy, ey, NW, bK); #c1(TRDN) c2(NP_001242950) c3(triadin isoform 4) c4(89373, 89374) c5(89375, 89376, 89377, 89378, 89379, 89380, 89381, 89382, 89383, 89384, 89385, 89386, 89387, 89388, 89389, 89390, 89391, 89392, 89393, 89394, 89395, 89396, 89397, 89398) c6( ) c7(cQd, cy, ey, NW, bK); #c1(TRDN) c2(NP_001242951) c3(triadin isoform 5) c4(89399, 89400) c5(89401, 89402, 89403, 89404, 89405, 89406, 89407, 89408, 89409, 89410, 89411, 89412, 89413, 89414, 89415, 89416, 89417, 89418, 89419, 89420, 89421, 89422, 89423, 89424) c6( ) c7(cQd, cy, ey, NW, bk); #c1(TRDN) c2(NP_006064) c3(triadin isoform 1) c4(161584, 161585, 161586, 161587) c5(161588, 161589, 161590, 161591, 161592, 161593, 161594, 161595, 161596, 161597, 161598, 161599, 161600, 161601, 161602, 161603, 161604, 161605) c6( ) c7(cQd, cy, ey, NW, bK); #c1(TRDN) c2(XP_011533684) c3(triadin isoform X1) c4(329741) c5( ) c6( ) c7(cdd, cy, ey, NW, bk); #c1(TREH) c2(NP_001287994) c3(trehalase isoform 2 precursor) c4(113329, 113330) c5(113331, 113332, 113333, 113334, 113335, 113336, 113337, 113338, 113339, 113340, 113341, 113342) c6( ) c7(bL, T, aj, eA, J, cQe, aBy, H, jf, je, aZs, eZ, bBG, kJ, bz, ck, vW, iM, eM, dy, R, L); #c1(TREH) c2(NP_009111) c3(trehalase isoform 1 precursor) c4(170750, 170751, 170752, 170753) c5(170754, 170755, 170756, 170757, 170758, 170759, 170760, 170761, 170762, 170763, 170764, 170765, 170766, 170767, 170768, 170769, 170770, 170771) c6( ) c7(bL, T, aj, eA, J, cQe, aBy, H, jf, je, aZs, eZ, bBG, kJ, bz, ck, vW, iM, eM, dy, R, L); #c1(TREH) c2(XP_011540866) c3(trehalase isoform X1) c4( ) c5(339396, 339397, 339398, 339399, 339400, 339401) c6( ) c7(bL, T, aj, eA, J, cQe, aBy, H, jf, je, aZs, eZ, bBG, kJ, bz, ck, vW, iM, eM, dy, R, L); #c1(TREM1) c2(NP_001229518) c3(triggering receptor expressed on myeloid cells 1 isoform 2 precursor) c4(84589) c5(84590, 84591, 84592, 84593, 84594, 84595) c6( ) c7(kV, oO, T, FR, sQ, LX, cL, C, ai, o, Tz, aDF, aT, ji, lQ, aWf, he, le, mR, aW, aoo, bg, hl, la, fe, aB, nN, iV, axp, bz, ba, ar, eM, cj, uo, at, wR, ut, dy, cMD, fD, y, ra, ao); #c1(TREM1) c2(NP_001229519) c3(triggering receptor expressed on myeloid cells 1 isoform 3 precursor) c4(84596) c5(84597, 84598, 84599, 84600, 84601, 84602) c6( ) c7(kV, oO, T, fR, sQ, LX, cL, C, ai, o, Tz, aDF, aT, ji, IQ, aWf, he, le, mR, aW, aoo, bg, hl, la, fe, aB, nN, iV, axp, bz, ba, ar, eM, cj, uo, at, wR, ut, dy, CMD, fD, y, ra, ao); #c1(TREM1) c2(NP_061113) c3(triggering receptor expressed on myeloid cells 1 isoform 1 precursor) c4(199158) c5(199159, 199160, 199161, 199162, 199163, 199164) c6( ) c7(kV, oO, T, fR, sQ, LX, cL, C, ai, o, Tz, aDF, aT, ji, lQ, aWf, he, le, mR, aW, aoo, bg, hl, la, fe, aB, nN, iV, axp, bz, ba, ar, eM, cj, uo, at, wR, ut, dy, cMD, fD, y, ra, ao); #c1(TREM2) c2(NP_001258750) c3 (triggering receptor expressed on myeloid cells 2 precursor isoform 2 precursor) c4(96488, 96489) c5(96490, 96491, 96492, 96493, 96494, 96495, 96496, 96497, 96498, 96499, 96500, 96501) c6( ) c7(oO, d, bb, c, af, hb, WY, OI, M, U, aaE, axQ, act, H, V, fK, dr); #c1(TREM2) c2(NP_061838) c3(triggering receptor expressed on myeloid cells 2 precursor isoform 1 precursor) c4(200731) c5(200732, 200733, 200734, 200735, 200736, 200737) c6( ) c7(oO, d, bb, c, af, hb, WY, OI, M, U, aaE, axQ, act, H, V, fK, dr); #c1(TREML1) c2(NP_001258736) c3(trem-like transcript 1 protein isoform b precursor) c4(96457) c5(96458, 96459, 96460, 96461, 96462) c6( ) c7(dc, C); #c1(TREML1) c2(NP_001258737) c3(trem-like transcript 1 protein isoform c precursor) c4(96463) c5(96464, 96465, 96466, 96467, 96468, 96469) c6( ) c7(dc, C); #c1(TREML1) c2(NP_835468) c3(trem-like transcript 1 protein isoform a precursor) c4(253539) c5(253540, 253541, 253542, 253543, 253544, 253545, 253546) c6( ) c7(dc, C); #c1(TREML2) c2(NP_079083) c3(trem-like transcript 2 protein precursor) c4(216922) c5(216923, 216924, 216925, 216926, 216927, 216928) c6( ) c7(cz); #c1(TRERF1) c2(XP_006715208) c3(transcriptional-regulating factor 1 isoform X2) c4(288825) c5(288826, 288827, 288828, 288829, 288830, 288831) c6( ) c7(aW, LC, bX, aT); #c1(TRERF1) c2(XP_006715210) c3 (transcriptional-regulating factor 1 isoform X10) c4(288832) c5(288833, 288834, 288835, 288836, 288837, 288838, 288839) c6( ) c7(aW, LC, bX, aT); #c1(TRERF1) c2(XP_011513052) c3(transcriptional-regulating factor 1 isoform X1) c4(304643) c5(304644, 304645, 304646, 304647, 304648, 304649) c6( ) c7(aW, LC, bX, aT); #c1(TREX1) c2(NP_009179) c3(three-prime repair exonuclease 1 isoform c) c4(171331, 171332) c5(171333, 171334, 171335, 171336, 171337, 171338, 171339, 171340, 171341, 171342, 171343, 171344, 171345, 171346, 171347) c6( ) c7(T, Ez, aC, uu, mC, hb, Dq, ds, h, K, cb, lR, cQg, eZ, cQf, ack, lo, g, aaO, pb, boN, lt, wR, aSu, fL, y, Ex); #c1(TREX1) c2(NP_057465) c3(three-prime repair exonuclease 1 isoform a) c4(191100) c5(191101, 191102, 191103, 191104, 191105, 191106, 191107, 191108) c6( ) c7(T, Ez, aC, uu, mC, hb, Dq, ds, h, K, cb, lR, cQg, eZ, cQf, ack, lo, g, aaO, pb, boN, lt, wR, aSu, fL, y, Ex); #c1(TREX1) c2(NP_338599) c3(three-prime repair exonuclease 1 isoform b) c4(228916) c5(228917, 228918, 228919, 228920, 228921, 228922, 228923, 228924, 228925) c6( ) c7(T, Ez, aC, uu, mC, hb, Dq, ds, h, K, cb, lR, cQg, eZ, cQf, ack, lo, g, aaO, pb, boN, lt, wR, aSu, fL, y, Ex); #c1(TREX2) c2(NP_542432) c3(three prime repair exonuclease 2) c4(232837, 232838, 232839) c5(232840, 232841, 232842, 232843, 232844, 232845, 232846, 232847, 232848, 232849, 232850, 232851, 232852, 232853, 232854, 232855, 232856, 232857, 232858, 232859, 232860, 232861, 232862, 232863, 232864, 232865, 232866, 232867, 232868, 232869, 232870, 232871, 232872, 232873, 232874, 232875, 232876) c6( ) c7(ad, eZ, bq); #c1(TRH) c2(NP_009048) c3(thyrotropin releasing hormone preproprotein) c4(170414) c5(170415, 170416, 170417, 170418, 170419, 170420) c6( ) c7(bL, T, cQh, cQj, WL, ha, bzb, bO, cz, cQ, bS, bRy, ct, cow, biO, JW, xt, dP, cF, HD, K, EL, dQ, he, NM, ae, uQ, aqa, yo, d, s, iU, Y, Fe, aq, lo, Z, cox, cm, bw, amt, pv, cQi, ky, dJ, bo, U, cj, hr, hR, gT, bt, B); #c1(TRHDE) c2(NP_037513) c3(thyrotropin-releasing hormone-degrading ectoenzyme) c4(175959) c5(175960, 175961, 175962, 175963, 175964, 175965) c6( ) c7(y, j); #c1(TRHR) c2(XP_011515565) c3(thyrotropin-releasing hormone receptor isoform X1) c4(308394) c5(308395, 308396, 308397, 308398, 308399, 308400, 308401, 308402, 308403, 308404) c6( ) c7(pv, aPW, cQl, yo, s, cQk, lT, cz, hR, v, gT, JW, qa, apL); #c1(TRIAP1) c2(NP_057483) c3(TP53-regulated inhibitor of apoptosis 1) c4(191143) c5(191144, 191145, 191146, 191147, 191148, 191149) c6( ) c7(fd); #c1(TRIB1) c2(NP_079471) c3(tribbles homolog 1 isoform 1) c4(218216) c5(218217, 218218, 218219, 218220, 218221, 218222) c6( ) c7(bL, ck, aC, ec, iT, cO, e, aw, M, fe, n, iY, v, j, bw, ei, f, bt, Qt); #c1(TRIB2) c2(NP_067675) c3(tribbles homolog 2) c4(209271) c5(209272, 209273, 209274, 209275, 209276, 209277, 209278, 209279) c6(209280, 209281) c7(T, yk, EQ, oH, j, qW, ad, cF, ae, af, iI, Y, fe, aq, g, iR, bw, ck, iT, cj, aS, bt); #c1(TRIB3) c2(NP_001288117) c3(tribbles homolog 3 isoform 1) c4( ) c5(113556, 113557, 113558, 113559, 113560, 113561, 113562, 113563, 113564, 113565, 113566, 113567) c6( ) c7(bL, T, dD, aC, ea, e, aw, sM, n, aj, dG, j, v, eq, q, J, ad, aT, K, ae, af, aW, r, bw, KM, ck, wE, mf, cS, ei, bt); #c1(TRIB3) c2(NP_001288130) c3(tribbles homolog 3 isoform 2) c4(113579, 113580, 113581) c5(113582, 113583, 113584, 113585, 113586, 113587) c6( ) c7(bL, T, dD, aC, ea, e, aw, sM, n, aj, dG, j, v, eq, q, J, ad, aT, K, ae, af, aW, r, bw, KM, ck, wE, mf, cS, ei, bt); #c1(TRIM10) c2(NP_006769) c3 (tripartite motif-containing protein 10 isoform 1) c4(167908, 167909, 167910) c5(167911, 167912, 167913, 167914, 167915, 167916, 167917, 167918, 167919, 167920, 167921, 167922, 167923, 167924, 167925, 167926, 167927, 167928, 167929, 167930, 167931, 167932, 167933, 167934, 167935, 167936, 167937, 167938, 167939, 167940, 167941, 167942, 167943, 167944, 167945, 167946, 167947, 167948, 167949, 167950, 167951, 167952, 167953, 167954, 167955, 167956, 167957, 167958, 167959, 167960, 167961, 167962, 167963, 167964, 167965, 167966, 167967, 167968, 167969, 167970, 167971) c6( ) c7(V, eZ, KU); #c1(TRIM10) c2(NP_439893) c3(tripartite motif-containing protein 10 isoform 2) c4(229137) c5(229138, 229139, 229140, 229141, 229142, 229143, 229144, 229145, 229146, 229147, 229148, 229149, 229150, 229151, 229152, 229153, 229154, 229155, 229156, 229157, 229158, 229159, 229160, 229161, 229162, 229163, 229164, 229165, 229166, 229167, 229168, 229169) c6( ) c7(V, eZ, KU); #c1(TRIM10) c2(XP_011512526) c3 (tripartite motif-containing protein 10 isoform X3) c4( ) c5(303164, 303165, 303166, 303167, 303168, 303169) c6( ) c7(V, eZ, KU); #c1(TRIM11) c2(NP_660215) c3(E3 ubiquitin-protein ligase TRIM11) c4(240559, 240560) c5(240561, 240562, 240563, 240564, 240565, 240566, 240567, 240568, 240569, 240570, 240571, 240572) c6( ) c7(T, d, eA, km); #c1(TRIM13) c2(NP_001007279) c3(E3 ubiquitin-protein ligase TRIM13 isoform 2) c4(22685, 22686, 22687, 22688) c5(22689, 22690, 22691, 22692, 22693, 22694, 22695, 22696, 22697, 22698, 22699, 22700, 22701, 22702, 22703, 22704, 22705, 22706, 22707) c6( ) c7(gV, T, dA, qb, jh, fd, v, ci, q, ad, eA, eW, aT, dL, ae, af, eL, PP, s, hG, aW, Y, OX, oV, r, lo, t, cm, ck, kT, mx, cj, iS, MJ, m, gT, bt, B); #c1(TRIM13) c2(NP_998755) c3(E3 ubiquitin-protein ligase TRIM13 isoform 1) c4( ) c5(265840, 265841, 265842, 265843, 265844, 265845, 265846, 265847, 265848, 265849, 265850, 265851, 265852, 265853, 265854, 265855, 265856, 265857, 265858, 265859, 265860) c6( ) c7(gV, T, dA, qb, jh, fd, v, ci, q, ad, eA, eW, aT, dL, ae, af, eL, PP, s, hG, aW, Y, OX, oV, r, lo, t, cm, ck, kT, mx, cj, iS, MJ, m, gT, bt, B); #c1(TRIM15) c2(NP_150232) c3(tripartite motif-containing protein 15) c4(227853, 227854, 227855, 227856, 227857) c5(227858, 227859, 227860, 227861, 227862, 227863, 227864, 227865, 227866, 227867, 227868, 227869, 227870, 227871, 227872, 227873, 227874, 227875, 227876, 227877, 227878, 227879, 227880, 227881, 227882, 227883, 227884, 227885, 227886, 227887, 227888, 227889, 227890, 227891, 227892, 227893, 227894, 227895, 227896, 227897, 227898, 227899, 227900, 227901, 227902, 227903, 227904, 227905, 227906, 227907, 227908, 227909, 227910, 227911, 227912, 227913, 227914, 227915, 227916, 227917, 227918, 227919, 227920, 227921, 227922, 227923, 227924) c6( ) c7(V, eZ, cr); #c1(TRIM15) c2(XP_011513289) c3(tripartite motif-containing protein 15 isoform X1) c4(305552) c5(305553, 305554, 305555, 305556, 305557, 305558, 305559, 305560, 305561, 305562, 305563, 305564, 305565, 305566, 305567, 305568, 305569, 305570, 305571, 305572, 305573, 305574, 305575, 305576, 305577, 305578) c6( ) c7(V, eZ, cr); #c1(TRIM15) c2(XP_011545633) c3(tripartite motif-containing protein 15 isoform X2) c4( ) c5(344553, 344554, 344555, 344556, 344557, 344558, 344559, 344560, 344561, 344562, 344563, 344564, 344565, 344566, 344567, 344568, 344569, 344570, 344571, 344572, 344573, 344574, 344575, 344576, 344577, 344578, 344579, 344580, 344581) c6( ) c7(V, eZ, cr); #c1(TRIM16) c2(NP_006461) c3(tripartite motif-containing protein 16) c4(165396) c5(165397, 165398, 165399, 165400, 165401, 165402, 165403, 165404, 165405, 165406, 165407) c6( ) c7(cm, fG, T, kS, jj); #c1(TRIM17) c2(NP_001020111) c3(E3 ubiquitin-protein ligase TRIM17 isoform 1) c4(28200, 28201) c5(28202, 28203, 28204, 28205, 28206, 28207, 28208, 28209, 28210, 28211, 28212, 28213, 28214, 28215, 28216) c6( ) c7(km, aF, aRk, aA); #c1(TRIM17) c2(NP_001128327) c3(E3 ubiquitin-protein ligase TRIM17 isoform 3) c4(49842, 49843) c5(49844, 49845, 49846, 49847, 49848, 49849, 49850, 49851, 49852, 49853, 49854, 49855, 49856, 49857, 49858, 49859, 49860, 49861) c6( ) c7(km, aF, aRk, aA); #c1(TRIM2) c2(NP_001123539) c3(tripartite motif-containing protein 2 isoform 2) c4(47458) c5(47459) c6( ) c7(V, bk, cQm); #c1(TRIM2) c2(NP_056086) c3(tripartite motif-containing protein 2 isoform 1) c4(185622) c5(185623, 185624, 185625, 185626, 185627, 185628, 185629, 185630, 185631, 185632, 185633, 185634) c6( ) c7(V, bk, cQm); #c1(TRIM21) c2(NP_003132) c3(E3 ubiquitin-protein ligase TRIM21) c4(135550, 135551) c5(135552, 135553, 135554, 135555, 135556, 135557, 135558, 135559, 135560, 135561, 135562, 135563, 135564) c6( ) c7(Ob, T, cQn, cQo, qb, fN, bB, ci, mC, q, ai, ft, xy, rw, NS, nI, aT, hD, h, K, lQ, azv, atZ, DY, dL, yo, Yj, s, aW, ff, lb, ih, nh, eZ, dN, aiT, lo, bJC, afi, NR, en, YZ, t, bz, ck, bF, Bv, bE, uw, hR, aYx, cQp, azy, dy, fL, R, y, jr); #c1(TRIM22) c2(NP_001186502) c3(E3 ubiquitin-protein ligase TRIM22 isoform 2) c4(77714, 77715) c5(77716, 77717, 77718, 77719, 77720, 77721, 77722, 77723, 77724, 77725, 77726, 77727) c6( ) c7(h, Kd, ds, fR); #c1(TRIM22) c2(NP_006065) c3(E3 ubiquitin-protein ligase TRIM22 isoform 1) c4(161606, 161607, 161608) c5(161609, 161610, 161611, 161612, 161613, 161614, 161615, 161616, 161617, 161618, 161619, 161620, 161621, 161622, 161623, 161624, 161625, 161626, 161627, 161628) c6( ) c7(h, Kd, ds, fR); #c1(TRIM23) c2(NP_001647) c3(E3 ubiquitin-protein ligase TRIM23 isoform alpha) c4(119714) c5(119715, 119716, 119717, 119718, 119719, 119720, 119721, 119722, 119723, 119724, 119725) c6( ) c7(T, aW, r, aT, ji, apx, q); #c1(TRIM23) c2(NP_150230) c3(E3 ubiquitin-protein ligase TRIM23 isoform beta) c4(227837) c5(227838, 227839, 227840, 227841, 227842, 227843) c6( ) c7(T, aW, r, aT, ji, apx, q); #c1(TRIM23) c2(NP_150231) c3(E3 ubiquitin-protein ligase TRIM23 isoform gamma) c4(227844, 227845) c5(227846, 227847, 227848, 227849, 227850, 227851, 227852) c6( ) c7(T, aW, r, aT, ji, apx, q); #c1(TRIM24) c2(NP_003843) c3(transcription intermediary factor 1-alpha isoform b) c4(141321, 141322) c5(141323, 141324, 141325, 141326, 141327, 141328, 141329, 141330, 141331, 141332, 141333, 141334, 141335) c6( ) c7(x, T, hd, Bv, nM, eM, aW, iK, aT, n, aq, k, iR, cm, bt, bz, B); #c1(TRIM24) c2(NP_056989) c3(transcription intermediary factor 1-alpha isoform a) c4(188348, 188349) c5(188350, 188351, 188352, 188353, 188354, 188355, 188356, 188357, 188358, 188359, 188360, 188361) c6( ) c7(x, T, hd, Bv, nM, eM, aW, iK, aT, n, aq, k, iR, cm, bt, bz, B); #c1(TRIM25) c2(NP_005073) c3(E3 ubiquitin/ISG15 ligase TRIM25) c4(152630) c5(152631, 152632, 152633, 152634, 152635, 152636, 152637, 152638, 152639, 152640) c6( ) c7(aW, ds, hL, aT, ci, g); #c1(TRIM26) c2(XP_005249435) c3 (tripartite motif-containing protein 26 isoform X1) c4(269305, 269306, 269307, 269308, 269309) c5(269310, 269311, 269312, 269313, 269314, 269315, 269316, 269317, 269318, 269319, 269320, 269321, 269322, 269323, 269324, 269325, 269326, 269327, 269328, 269329, 269330, 269331, 269332, 269333, 269334, 269335, 269336, 269337, 269338, 269339, 269340, 269341, 269342, 269343, 269344, 269345, 269346, 269347, 269348, 269349, 269350, 269351, 269352, 269353, 269354, 269355, 269356, 269357, 269358, 269359, 269360, 269361, 269362, 269363, 269364, 269365, 269366, 269367, 269368, 269369, 269370, 269371, 269372, 269373, 269374, 269375, 269376, 269377, 269378, 269379, 269380, 269381, 269382, 269383, 269384, 269385, 269386, 269387, 269388, 269389, 269390, 269391, 269392, 269393, 269394, 269395, 269396, 269397, 269398, 269399, 269400, 269401, 269402, 269403, 269404, 269405, 269406, 269407, 269408, 269409, 269410, 269411, 269412, 269413, 269414, 269415, 269416, 269417, 269418, 269419, 269420, 269421, 269422, 269423, 269424, 269425, 269426, 269427, 269428, 269429, 269430, 269431) c6( ) c7(Le, T, wu, al, jh, aA, Hn, n, bX, V, eW, hX, jz, F, KN, rO, wm, hH, Df, vT, Jl, hl, Lh, eZ, aq, aF, g, iR, f, KQ, KM, bF, iO, gk, kQ, bC, be, hE, KY, B); #c1(TRIM26) c2(XP_011513161) c3(tripartite motif-containing protein 26 isoform X2) c4( ) c5(305015, 305016, 305017, 305018, 305019, 305020, 305021, 305022, 305023, 305024, 305025, 305026, 305027, 305028, 305029, 305030, 305031, 305032, 305033, 305034, 305035, 305036, 305037, 305038, 305039, 305040, 305041, 305042, 305043, 305044, 305045, 305046, 305047, 305048, 305049, 305050, 305051, 305052, 305053, 305054, 305055, 305056, 305057, 305058, 305059, 305060, 305061, 305062, 305063, 305064, 305065, 305066, 305067, 305068, 305069, 305070, 305071, 305072, 305073, 305074, 305075, 305076, 305077, 305078, 305079, 305080, 305081, 305082, 305083, 305084, 305085, 305086, 305087, 305088, 305089, 305090, 305091, 305092, 305093, 305094, 305095, 305096, 305097, 305098, 305099, 305100, 305101) c6( ) c7(Le, T, wu, al, jh, aA, Hn, n, bX, V, eW, hX, jz, F, KN, rO, wm, hH, Df, vT, Jl, hl, Lh, eZ, aq, aF, g, iR, f, KQ, KM, bF, iO, gk, kQ, bC, be, HE, KY, B); #c1(TRIM27) c2(NP_006501) c3(zinc finger protein RFP) c4(165757, 165758, 165759, 165760, 165761, 165762, 165763, 165764) c5(165765, 165766, 165767, 165768, 165769, 165770, 165771, 165772, 165773, 165774, 165775, 165776, 165777, 165778, 165779, 165780, 165781, 165782, 165783, 165784, 165785, 165786, 165787, 165788, 165789, 165790, 165791, 165792, 165793, 165794, 165795, 165796, 165797, 165798, 165799, 165800, 165801, 165802, 165803, 165804, 165805, 165806, 165807, 165808, 165809, 165810, 165811, 165812, 165813, 165814, 165815, 165816, 165817, 165818, 165819, 165820, 165821, 165822, 165823, 165824, 165825, 165826, 165827, 165828, 165829, 165830, 165831, 165832, 165833, 165834, 165835, 165836, 165837, 165838, 165839, 165840, 165841, 165842, 165843, 165844, 165845, 165846, 165847, 165848, 165849, 165850, 165851, 165852, 165853, 165854, 165855, 165856, 165857, 165858, 165859, 165860, 165861, 165862) c6( ) c7(T, bkF, yk, bB, eA, afb, aE, adX, dL, K, eL, Y, eH, eZ, cl, eF, nM, adZ, wO, hk, hL, it); #c1(TRIM28) c2(NP_005753) c3(transcription intermediary factor 1-beta) c4(159026, 159027) c5(159028, 159029, 159030, 159031, 159032, 159033, 159034, 159035, 159036, 159037, 159038, 159039, 159040) c6( ) c7(av, T, eM, aW, Y, aT, aA, n, h, aF, bC, g, bz); #c1(TRIM29) c2(NP_036233) c3(tripartite motif-containing protein 29) c4(172522, 172523, 172524, 172525, 172526, 172527) c5(172528, 172529, 172530, 172531, 172532, 172533, 172534, 172535, 172536, 172537, 172538, 172539, 172540, 172541, 172542, 172543, 172544, 172545, 172546, 172547, 172548, 172549, 172550, 172551, 172552, 172553) c6( ) c7(T, aE, aW, if, ff, aT, n, kp, bC, en, qN, ad); #c1(TRIM29) c2(XP_005271547) c3 (tripartite motif-containing protein 29 isoform X4) c4( ) c5(283462, 283463, 283464, 283465, 283466, 283467) c6( ) c7(T, aE, aW, if, ff, aT, n, kp, bC, en, qN, ad); #c1(TRIM29) c2(XP_011541033) c3(tripartite motif-containing protein 29 isoform X1) c4( ) c5(339564, 339565, 339566, 339567, 339568) c6( ) c7(T, aE, aW, if, ff, aT, n, kp, bC, en, qN, ad); #c1(TRIM3) c2(XP_011518146) c3(tripartite motif-containing protein 3 isoform X1) c4(312128, 312129, 312130, 312131, 312132) c5(312133, 312134, 312135, 312136, 312137, 312138, 312139, 312140, 312141, 312142, 312143, 312144, 312145, 312146, 312147, 312148, 312149, 312150, 312151, 312152, 312153, 312154, 312155, 312156, 312157, 312158, 312159, 312160, 312161, 312162, 312163, 312164, 312165, 312166, 312167, 312168, 312169, 312170, 312171) c6( ) c7(dB, eA, v, d, K); #c1(TRIM3) c2(XP_011518147) c3(tripartite motif-containing protein 3 isoform X2) c4(312172) c5(312173, 312174, 312175, 312176, 312177, 312178) c6( ) c7(dB, eA, v, d, K); #c1(TRIM31) c2(NP_008959) c3(E3 ubiquitin-protein ligase TRIM31) c4(169809, 169810, 169811, 169812, 169813, 169814, 169815, 169816) c5(169817, 169818, 169819, 169820, 169821, 169822, 169823, 169824, 169825, 169826, 169827, 169828, 169829, 169830, 169831, 169832, 169833, 169834, 169835, 169836, 169837, 169838, 169839, 169840, 169841, 169842, 169843, 169844, 169845, 169846, 169847, 169848, 169849, 169850, 169851, 169852, 169853, 169854, 169855, 169856, 169857, 169858, 169859, 169860, 169861, 169862, 169863, 169864, 169865, 169866, 169867, 169868, 169869, 169870, 169871, 169872, 169873, 169874, 169875, 169876, 169877, 169878, 169879, 169880, 169881, 169882, 169883, 169884, 169885, 169886, 169887, 169888, 169889, 169890, 169891, 169892, 169893, 169894, 169895, 169896, 169897, 169898, 169899, 169900, 169901, 169902, 169903, 169904, 169905, 169906, 169907, 169908, 169909, 169910, 169911, 169912, 169913, 169914, 169915, 169916) c6( ) c7(T, eM, cQq, Jl, aW, dE, bE, cr, aT, aA, bB, yy, he, aF, eZ, nA, ei, nS, hH); #c1(TRIM31) c2(XP_011512567) c3(E3 ubiquitin-protein ligase TRIM31 isoform X5) c4(303475) c5( ) c6( ) c7(T, eM, cQq, Jl, aW, dE, bE, cr, aT, aA, bB, yy, he, af, eZ, nA, ei, nS, hH); #c1(TRIM31) c2(XP_011512568) c3(E3 ubiquitin-protein ligase TRIM31 isoform X4) c4( ) c5(303476, 303477, 303478, 303479, 303480, 303481) c6( ) c7(T, eM, cQq, Jl, aW, dE, bE, cr, aT, aA, bB, yy, he, aF, eZ, nA, ei, nS, hH); #c1(TRIM32) c2(XP_011516700) c3(E3 ubiquitin-protein ligase TRIM32 isoform X1) c4(310131, 310132) c5(310133, 310134, 310135, 310136, 310137, 310138, 310139, 310140, 310141, 310142, 310143, 310144, 310145, 310146, 310147, 310148, 310149, 310150) c6( ) c7(ck, T, lR, eD, ff, xs, oj, km, cr, yT, aiW, v, en, Tp, adg, rz, iK, cnS); #c1(TRIM34) c2(NP_067629) c3(tripartite motif-containing protein 34 isoform 1) c4(209090) c5(209091, 209092, 209093, 209094, 209095, 209096, 209097, 209098, 209099, 209100, 209101, 209102) c6( ) c7(h); #c1(TRIM35) c2(NP_741983) c3(tripartite motif-containing protein 35 isoform 1) c4(247523, 247524) c5(247525, 247526, 247527, 247528, 247529, 247530, 247531, 247532, 247533, 247534, 247535, 247536, 247537) c6( ) c7(ck, B); #c1(TRIM36) c2(NP_001017397) c3(E3 ubiquitin-protein ligase TRIM36 isoform 2) c4(26405) c5(26406, 26407, 26408, 26409, 26410, 26411) c6( ) c7(ad); #c1(TRIM36) c2(NP_001017398) c3(E3 ubiquitin-protein ligase TRIM36 isoform 3) c4(26412) c5(26413, 26414, 26415, 26416, 26417, 26418) c6( ) c7(ad); #c1(TRIM36) c2(NP_061170) c3(E3 ubiquitin-protein ligase TRIM36 isoform 1) c4(199726) c5(199727, 199728, 199729, 199730, 199731, 199732) c6( ) c7(ad); #c1(TRIM37) c2(NP_056109) c3(E3 ubiquitin-protein ligase TRIM37) c4(185747) c5(185748, 185749, 185750, 185751, 185752, 185753, 185754, 185755) c6( ) c7(adf, eM, cQq, aW, Az, aT, mE, ey, ei, t, nS, er, ae, ad); #c1(TRIM37) c2(XP_005257444) c3(E3 ubiquitin-protein ligase TRIM37 isoform X8) c4(275114) c5(275115, 275116, 275117, 275118, 275119, 275120) c6( ) c7(adf, eM, cQq, aW, Az, aT, mE, ey, ei, t, nS, er, ae, ad); #c1(TRIM37) c2(XP_005257447) c3(E3 ubiquitin-protein ligase TRIM37 isoform X10) c4(275121) c5(275122, 275123, 275124, 275125, 275126) c6( ) c7(adf, eM, cQq, aW, Az, aT, mE, ey, ei, t, nS, er, ae, ad); #c1(TRIM37) c2(XP_011523134) c3(E3 ubiquitin-protein ligase TRIM37 isoform X2) c4( ) c5(317678, 317679) c6( ) c7(adf, eM, cQq, aW, Az, aT, mE, ey, ei, t, nS, er, ae, ad); #c1(TRIM37) c2(XP_011523138) c3(E3 ubiquitin-protein ligase TRIM37 isoform X7) c4( ) c5(317680) c6( ) c7(adf, eM, cQq, aW, Az, aT, mE, ey, ei, t, nS, er, ae, ad); #c1(TRIM38) c2(XP_005248857) c3(E3 ubiquitin-protein ligase TRIM38 isoform X1) c4(268656) c5(268657, 268658, 268659, 268660, 268661, 268662, 268663, 268664, 268665, 268666, 268667, 268668, 268669, 268670, 268671) c6( ) c7(ih, eZ); #c1(TRIM38) c2(XP_011512534) c3(E3 ubiquitin-protein ligase TRIM38 isoform X2) c4( ) c5(303193) c6( ) c7(ih, eZ); #c1(TRIM39) c2(NP_067076) c3(E3 ubiquitin-protein ligase TRIM39 isoform 1) c4(208836, 208837, 208838, 208839) c5(208840, 208841, 208842, 208843, 208844, 208845, 208846, 208847, 208848, 208849, 208850, 208851, 208852, 208853, 208854, 208855, 208856, 208857, 208858, 208859, 208860, 208861, 208862, 208863, 208864, 208865, 208866, 208867, 208868, 208869, 208870, 208871, 208872, 208873, 208874, 208875, 208876, 208877, 208878, 208879, 208880, 208881, 208882, 208883, 208884, 208885, 208886, 208887, 208888, 208889, 208890, 208891, 208892, 208893, 208894, 208895, 208896, 208897, 208898, 208899, 208900, 208901, 208902, 208903, 208904, 208905, 208906, 208907, 208908, 208909, 208910, 208911, 208912, 208913, 208914, 208915, 208916) c6( ) c7(eZ); #c1(TRIM39) c2(NP_742013) c3(E3 ubiquitin-protein ligase TRIM39 isoform 2) c4(247597, 247598, 247599, 247600, 247601) c5(247602, 247603, 247604, 247605, 247606, 247607, 247608, 247609, 247610, 247611, 247612, 247613, 247614, 247615, 247616, 247617, 247618, 247619, 247620, 247621, 247622, 247623, 247624, 247625, 247626, 247627, 247628, 247629, 247630, 247631, 247632, 247633, 247634, 247635, 247636, 247637, 247638, 247639, 247640, 247641, 247642, 247643, 247644, 247645, 247646, 247647, 247648, 247649, 247650, 247651, 247652, 247653, 247654, 247655, 247656, 247657, 247658, 247659, 247660, 247661, 247662, 247663, 247664, 247665, 247666, 247667, 247668, 247669, 247670, 247671, 247672, 247673, 247674, 247675, 247676, 247677, 247678, 247679, 247680, 247681, 247682, 247683, 247684, 247685, 247686, 247687, 247688, 247689, 247690, 247691, 247692, 247693, 247694, 247695, 247696, 247697, 247698, 247699, 247700, 247701, 247702, 247703, 247704, 247705, 247706, 247707, 247708, 247709, 247710, 247711, 247712, 247713, 247714, 247715, 247716, 247717, 247718, 247719, 247720, 247721, 247722, 247723, 247724, 247725, 247726, 247727, 247728, 247729, 247730, 247731, 247732, 247733, 247734, 247735, 247736, 247737, 247738, 247739, 247740, 247741, 247742, 247743, 247744, 247745, 247746, 247747, 247748, 247749, 247750, 247751, 247752, 247753, 247754, 247755, 247756, 247757, 247758, 247759, 247760, 247761, 247762, 247763, 247764, 247765, 247766, 247767, 247768, 247769, 247770, 247771, 247772, 247773, 247774, 247775, 247776, 247777, 247778, 247779, 247780, 247781, 247782, 247783, 247784, 247785, 247786, 247787, 247788, 247789, 247790, 247791, 247792, 247793, 247794, 247795, 247796, 247797, 247798, 247799, 247800, 247801, 247802, 247803, 247804, 247805, 247806, 247807, 247808, 247809, 247810, 247811, 247812, 247813, 247814, 247815, 247816, 247817, 247818, 247819, 247820, 247821, 247822, 247823, 247824, 247825, 247826, 247827, 247828, 247829, 247830, 247831, 247832, 247833, 247834, 247835, 247836, 247837, 247838, 247839, 247840, 247841, 247842, 247843, 247844, 247845, 247846, 247847, 247848, 247849, 247850, 247851, 247852, 247853, 247854, 247855, 247856, 247857, 247858, 247859, 247860, 247861, 247862, 247863, 247864, 247865, 247866, 247867, 247868, 247869, 247870, 247871, 247872, 247873, 247874, 247875, 247876) c6( ) c7(eZ); #c1(TRIM40) c2(NP_001273562) c3(tripartite motif-containing protein 40 isoform a) c4(105241) c5(105242, 105243, 105244, 105245, 105246, 105247, 105248, 105249, 105250, 105251, 105252, 105253, 105254, 105255, 105256, 105257, 105258, 105259, 105260, 105261, 105262, 105263, 105264, 105265, 105266, 105267, 105268, 105269, 105270, 105271, 105272, 105273, 105274, 105275, 105276, 105277) c6( ) c7(V, eZ, cr); #c1(TRIM40) c2(NP_619645) c3(tripartite motif-containing protein 40 isoform b) c4(236960, 236961, 236962) c5(236963, 236964, 236965, 236966, 236967, 236968, 236969, 236970, 236971, 236972, 236973, 236974, 236975, 236976, 236977, 236978, 236979, 236980, 236981, 236982, 236983, 236984, 236985, 236986, 236987, 236988, 236989, 236990, 236991, 236992, 236993, 236994, 236995, 236996, 236997, 236998, 236999) c6( ) c7(V, eZ, cr); #c1(TRIM40) c2(XP_011545906) c3(tripartite motif-containing protein 40 isoform X2) c4(344791) c5(344792, 344793, 344794, 344795, 344796, 344797, 344798, 344799, 344800, 344801) c6( ) c7(V, eZ, cr); #c1(TRIM40) c2(XP_011546687) c3(tripartite motif-containing protein 40 isoform X2) c4(345471) c5(345472, 345473, 345474, 345475, 345476, 345477, 345478, 345479, 345480, 345481) c6( ) c7(V, eZ, cr); #c1(TRIM42) c2(NP_689829) c3(tripartite motif-containing protein 42) c4(244065) c5(244066, 244067, 244068, 244069, 244070, 244071) c6( ) c7(V, m); #c1(TRIM44) c2(NP_060053) c3(tripartite motif-containing protein 44) c4(193887) c5(193888, 193889, 193890, 193891, 193892, 193893) c6( ) c7(n, T, aF, aA, aT); #c1(TRIM5) c2(NP_149023) c3(tripartite motif-containing protein 5 isoform alpha) c4(226791, 226792) c5(226793, 226794, 226795, 226796, 226797, 226798, 226799, 226800, 226801, 226802, 226803, 226804, 226805) c6( ) c7(ck, Ke, fR, ih, h, lo, gn, V); #c1(TRIM5) c2(NP_149083) c3(tripartite motif-containing protein 5 isoform gamma) c4(227055) c5(227056, 227057, 227058, 227059, 227060, 227061) c6( ) c7(ck, Ke, fR, ih, h, lo, gn, V); #c1(TRIM5) c2(NP_149084) c3(tripartite motif-containing protein 5 isoform delta) c4(227062, 227063) c5(227064, 227065, 227066, 227067, 227068, 227069, 227070, 227071, 227072, 227073, 227074, 227075, 227076, 227077, 227078, 227079) c6( ) c7(ck, Ke, fR, ih, h, Io, gn, V); #c1(TRIM5) c2(XP_006718421) c3(tripartite motif-containing protein 5 isoform X7) c4( ) c5(291842, 291843, 291844, 291845, 291846, 291847, 291848, 291849, 291850, 291851, 291852, 291853) c6( ) c7(ck, Ke, fR, ih, h, lo, gn, V); #c1(TRIM5) c2(XP_011518727) c3(tripartite motif-containing protein 5 isoform X4) c4( ) c5(313136) c6( ) c7(ck, Ke, fR, ih, h, lo, gn, V); #c1(TRIMS) c2(XP_011518729) c3(tripartite motif-containing protein 5 isoform X6) c4(313137, 313138, 313139) c5(313140, 313141, 313142, 313143, 313144, 313145) c6( ) c7(ck, Ke, fR, ih, h, lo, gn, V); #c1(TRIM50) c2(NP_835226) c3(E3 ubiquitin-protein ligase TRIM50 isoform a) c4(253389) c5(253390, 253391, 253392, 253393, 253394, 253395, 253396, 253397, 253398, 253399) c6( ) c7(alC); #c1(TRIM56) c2(NP_112223) c3(E3 ubiquitin-protein ligase TRIM56) c4(220957) c5(220958, 220959, 220960, 220961, 220962, 220963) c6( ) c7(T, vi); #c1(TRIM58) c2(NP_056246) c3(E3 ubiquitin-protein ligase TRIM58) c4(186705) c5(186706, 186707, 186708, 186709, 186710, 186711, 186712) c6( ) c7(cz); #c1(TRIM59) c2(NP_775107) c3(tripartite motif-containing protein 59) c4(249543, 249544, 249545, 249546, 249547, 249548) c5(249549, 249550, 249551, 249552, 249553, 249554, 249555, 249556, 249557, 249558, 249559, 249560, 249561, 249562, 249563, 249564, 249565, 249566, 249567, 249568, 249569, 249570, 249571, 249572, 249573, 249574, 249575, 249576, 249577, 249578, 249579, 249580, 249581, 249582, 249583, 249584) c6( ) c7(ae, ad, aF, aA); #c1(TRIM62) c2(XP_011540007) c3(E3 ubiquitin-protein ligase TRIM62 isoform X1) c4(338327) c5(338328, 338329, 338330, 338331, 338332, 338333) c6( ) c7(T, aW, aT, n, ci, bz); #c1(TRIM66) c2(NP_055633) c3 (tripartite motif-containing protein 66) c4(182401) c5(182402, 182403, 182404, 182405, 182406) c6( ) c7(v); #c1(TRIM66) c2(XP_011518813) c3(tripartite motif-containing protein 66 isoform X5) c4( ) c5(313201, 313202) c6( ) c7(v); #c1(TRIM68) c2(NP_001291425) c3(E3 ubiquitin-protein ligase TRIM68 isoform 2) c4( ) c5(116010, 116011, 116012, 116013, 116014, 116015) c6( ) c7(bL, ae, ad, fN, eZ); #c1(TRIM68) c2(NP_060543) c3(E3 ubiquitin-protein ligase TRIM68 isoform 1) c4(196476, 196477) c5(196478, 196479, 196480, 196481, 196482, 196483, 196484) c6( ) c7(bL, ae, ad, fN, eZ); #c1(TRIM69) c2(NP_001288073) c3(E3 ubiquitin-protein ligase TRIM69 isoform c) c4( ) c5(113491, 113492, 113493, 113494, 113495, 113496, 113497, 113498) c6( ) c7(Dw, wV, C, aBl, atK, eW, cF, aE, ds, cU, cy, pi, eu, lo, hp, cMm, pm, ac, NL, fD, hh, KS); #c1(TRIM69) c2(NP_001288074) c3(E3 ubiquitin-protein ligase TRIM69 isoform d) c4(113499) c5(113500, 113501, 113502, 113503, 113504) c6( ) c7(Dw, wV, C, aBl, atK, eW, cF, aE, ds, cU, cy, pi, eu, lo, hp, cMm, pm, ac, NL, fD, hh, kS); #c1(TRIM69) c2(NP_542783) c3(E3 ubiquitin-protein ligase TRIM69 isoform b) c4(233063, 233064) c5(233065, 233066, 233067, 233068, 233069, 233070, 233071, 233072, 233073, 233074) c6( ) c7(Dw, wV, C, aBl, atK, eW, cF, aE, ds, cU, cy, pi, eu, lo, hp, cMm, pm, ac, NL, fD, hh, kS); #c1(TRIM69) c2(NP_892030) c3(E3 ubiquitin-protein ligase TRIM69 isoform a) c4(257682) c5(257683, 257684, 257685, 257686, 257687, 257688, 257689, 257690, 257691, 257692) c6( ) c7(Dw, wV, C, aBl, atK, eW, cF, aE, ds, cU, cy, pi, eu, Io, hp, cMm, pm, ac, NL, fD, hh, KS); #c1(TRIM6-TRIM34) c2(NP_001003819) c3(tripartite motif-containing 6 and tripartite motif-containing 34) c4(20206) c5(20207, 20208, 20209, 20210, 20211, 20212, 20213, 20214, 20215, 20216, 20217, 20218, 20219) c6( ) c7(h); #c1(TRIM71) c2(NP_001034200) c3(E3 ubiquitin-protein ligase TRIM71) c4(31581) c5(31582, 31583, 31584, 31585, 31586, 31587) c6( ) c7(iR, aq, B); #c1(TRIM72) c2(NP_001008275) c3(tripartite motif-containing protein 72) c4(23131) c5(23132, 23133, 23134, 23135, 23136, 23137) c6( ) c7(cd, xs, eZ, oj); #c1(TRIM73) c2(NP_944606) c3(tripartite motif-containing protein 73) c4(260649, 260650) c5(260651, 260652, 260653, 260654, 260655, 260656, 260657, 260658, 260659, 260660, 260661, 260662, 260663, 260664, 260665, 260666) c6( ) c7(alC); #c1(TRIM74) c2(XP_011514491) c3(tripartite motif-containing protein 74 isoform X2) c4(307196, 307197) c5(307198, 307199, 307200, 307201, 307202, 307203, 307204, 307205, 307206, 307207, 307208, 307209) c6( ) c7(alC); #c1(TRIM8) c2(NP_112174) c3(probable E3 ubiquitin-protein ligase TRIM8) c4(220610, 220611) c5(220612, 220613, 220614, 220615, 220616, 220617, 220618, 220619, 220620, 220621, 220622, 220623) c6( ) c7(ac); #c1(TRIM9) c2(NP_055978) c3(E3 ubiquitin-protein ligase TRIM9 isoform 1) c4(184857) c5(184858, 184859, 184860, 184861, 184862, 184863) c6( ) c7(ak, j, qe, ER, fr); #c1(TRIM9) c2(NP_443210) c3(E3 ubiquitin-protein ligase TRIM9 isoform 2) c4(229936) c5(229937, 229938, 229939, 229940, 229941, 229942) c6( ) c7(ak, j, qe, ER, fr); #c1(TRIM9) c2(XP_011534691) c3(E3 ubiquitin-protein ligase TRIM9 isoform X1) c4(331186) c5(331187, 331188, 331189, 331190, 331191, 331192) c6( ) c7(ak, j, qe, ER, fr); #c1(TRIO) c2(NP_009049) c3(triple functional domain protein) c4(170421, (70422) c5(170423, 170424, 170425, 170426, 170427, 170428, 170429, 170430, 170431, 170432, 170433, 170434, 170435, 170436, 170437, 170438, 170439, 170440, 170441, 170442, 170443, 170444, 170445, 170446) c6( ) c7(ZL, d, aq, dL, hP, ci, bt, eL); #c1(TRIO) c2(XP_011512413) c3(triple functional domain protein isoform X4) c4(303105, 303106) c5( ) c6( ) c7(ZL, d, aq, dL, hP, ci, bt, eL); #c1(TRIO) c2(XP_011512414) c3(triple functional domain protein isoform X5) c4(303107) c5(303108, 303109, 303110, 303111, 303112, 303113) c6( ) c7(ZL, d, aq, dL, hP, ci, bt, eL); #c1(TRIO) c2(XP_011512415) c3(triple functional domain protein isoform X6) c4( ) c5(303114) c6( ) c7(ZL, d, aq, dL, hP, ci, bt, eL); #c1(TRIOBP) c2(NP_001034230) c3(TRIO and F-actin-binding protein isoform 6) c4(31620, 31621, 31622, 31623) c5(31624, 31625, 31626, 31627, 31628, 31629, 31630, 31631, 31632, 31633, 31634, 31635) c6( ) c7(cQr, Bh); #c1(TRIOBP) c2(NP_008963) c3(TRIO and F-actin-binding protein isoform 1) c4(169938) c5(169939, 169940, 169941, 169942, 169943, 169944, 169945) c6( ) c7(cQr, Bh); #c1(TRIOBP) c2(NP_619538) c3(TRIO and F-actin-binding protein isoform 2) c4(236847) c5(236848, 236849, 236850, 236851, 236852, 236853, 236854, 236855, 236856, 236857, 236858, 236859, 236860, 236861, 236862, 236863, 236864, 236865, 236866) c6( ) c7(cQr, Bh); #c1(TRIP10) c2(NP_001275891) c3(cdc42-interacting protein 4 isoform 1) c4(107297) c5(107298, 107299, 107300, 107301, 107302, 107303) c6( ) c7(T, rR, s, dB, aW, if, eX, cj, aT, aq, eV, fD, qN, iR, axp, aDF); #c1(TRIP10) c2(NP_001275892) c3(cdc42-interacting protein 4 isoform 3) c4(107304) c5(107305, 107306, 107307, 107308, 107309, 107310) c6( ) c7(T, rR, s, dB, aW, if, eX, cj, aT, aq, eV, fD, qN, iR, axp, aDF); #c1(TRIP10) c2(NP_004231) c3(cdc42-interacting protein 4 isoform 2) c4(144494) c5(144495, 144496, 144497, 144498, 144499, 144500) c6( ) c7(T, rR, s, dB, aW, if, eX, cj, al, aq, eV, fD, qN, iR, axp, aDF); #c1(TRIP11) c2(NP_004230) c3(thyroid receptor-interacting protein (1) c4(144478, 144479) c5(144480, 144481, 144482, 144483, 144484, 144485, 144486, 144487, 144488, 144489, 144490, 144491, 144492, 144493) c6( ) c7(bt, bP, cQs); #c1(TRIP13) c2(NP_001159732) c3(pachytene checkpoint protein 2 homolog isoform 2) c4( ) c5(64643, 64644, 64645, 64646, 64647, 64648) c6( ) c7(UR, n, QK, xR, XD); #c1(TRIP13) c2(NP_004228) c3(pachytene checkpoint protein 2 homolog isoform 1) c4(144465, 144466) c5(144467, 144468, 144469, 144470, 144471, 144472, 144473, 144474, 144475) c6(144476, 144477) c7(UR, n, QK, xR, XD); #c1(TRIP4) c2(NP_057297) c3(activating signal cointegrator 1) c4(189956) c5(189957, 189958, 189959, 189960, 189961, 189962, 189963) c6( ) c7(bns, aW, beo, aT); #c1(TRIP6) c2(XP_011514847) c3(thyroid receptor-interacting protein 6 isoform X1) c4(307590) c5(307591, 307592, 307593, 307594, 307595, 307596, 307597, 307598) c6( ) c7(T, d, ja, CQ, ci, K); #c1(TRIQK) c2(XP_011515284) c3(triple QxxK/R motif-containing protein isoform X1) c4(308118, 308119) c5(308120, 308121, 308122, 308123, 308124, 308125, 308126, 308127, 308128, 308129, 308130, 308131, 308132, 308133, 308134, 308135, 308136, 308137, 308138, 308139, 308140, 308141, 308142, 308143, 308144, 308145, 308146, 308147, 308148, 308149, 308150, 308151, 308152, 308153, 308154, 308155, 308156, 308157, 308158, 308159, 308160, 308161, 308162, 308163) c6( ) c7(j, an); #c1(TRMT1) c2(NP_001136026) c3(tRNA (guanine(26)-N(2)-dimethyltransferase isoform 2) c4(54051) c5(54052, 54053, 54054, 54055, 54056, 54057) c6( ) c7(nA); #c1(TRMT1) c2(NP_060192) c3(tRNA (guanine(26)-N(2))-dimethyltransferase isoform 1) c4(194707) c5(194708, 194709, 194710, 194711, 194712, 194713, 194714, 194715, 194716, 194717) c6( ) c7(nA); #c1(TRMT1DA) c2(NP_001128137) c3(tRNA methyltransferase 10 homolog A) c4(49611, 49612, 49613) c5(49614, 49615, 49616, 49617, 49618, 49619, 49620, 49621, 49622, 49623, 49624, 49625, 49626, 49627) c6( ) c7(QK, aj, aij, J); #c1(TRMT1DA) c2(XP_006714481) c3(tRNA methyltransferase 10 homolog A isoform X2) c4( ) c5(287864, 287865, 287866, 287867, 287868, 287869, 287870, 287871, 287872, 287873, 287874, 287875) c6( ) c7(QK, aj, aij, J); #c1(TRMT12) c2(NP_060426) c3(tRNA wybutosine-synthesizing protein 2 homolog) c4(195910) c5(195911, 195912, 195913, 195914, 195915, 195916) c6( ) c7(aW, aT); #c1(TRMT44) c2(NP_689757) c3(probable tRNA (uracil-O(2)-)-methyltransferase) c4(243881) c5(243882, 243883, 243884, 243885, 243886, 243887) c6( ) c7(cj); #c1(TRMT44) c2(XP_011511711) c3(probable tRNA (uracil-O(2)-)-methyltransferase isoform X5) c4(302432) c5(302433, 302434, 302435, 302436, 302437, 302438) c6( ) c7(cj); #c1(TRMT5) c2(NP_065861) c3(tRNA (guanine(37)-NI)-methyltransferase) c4(205944) c5(205945, 205946, 205947, 205948, 205949, 205950) c6( ) c7(fq); #c1(TRMU) c2(NP_001269711) c3(mitochondrial tRNA-specific 2-thiouridylase 1 isoform d) c4( ) c5(102682) c6( ) c7(bL, gk, cc, ac, mG, al, hl, cQu, cQt, bgR, ajU); #c1(TRMU) c2(NP_060476) c3(mitochondrial tRNA-specific 2-thiouridylase 1 isoform a) c4(196129, 196130) c5(196131, 196132, 196133, 196134, 196135, 196136, 196137, 196138, 196139) c6( ) c7(bL, gk, cc, ac, mG, al, hl, cQu, cQt, bgR, ajU); #c1(TRMU) c2(XP_005261738) c3(mitochondrial tRNA-specific 2-thiouridylase 1 isoform X6) c4( ) c5(277594) c6( ) c7(bL, gk, cc, ac, mG, al, hl, cQu, cQt, bgR, ajU); #c1(TRMU) c2(XP_011528575) c3(mitochondrial tRNA-specific 2-thiouridylase 1 isoform X4) c4(323725, 323726) c5(323727, 323728, 323729, 323730, 323731, 323732, 323733, 323734, 323735, 323736, 323737, 323738, 323739, 323740, 323741, 323742, 323743, 323744, 323745, 323746, 323747, 323748, 323749, 323750) c6( ) c7(bL, gk, cc, ac, mG, al, hl, cQu, cQt, bgR, ajU); #c1(TRMU) c2(XP_011528577) c3(mitochondrial tRNA-specific 2-thiouridylase 1 isoform X5) c4( ) c5(323751) c6( ) c7(bL, gk, cc, ac, mG, al, hl, cQu, cQt, bgR, ajU); #c1(TRO) c2(NP_001034794) c3(trophinin isoform 5) c4(32279, 32280, 32281) c5(32282, 32283, 32284, 32285, 32286, 32287) c6( ) c7(dP, Vs, T, hB, afc, io, aq, Ld, bu, iR, t, bz, B); #c1(TRO) c2(NP_001258112) c3(trophinin isoform 6) c4( ) c5(95964, 95965, 95966, 95967, 95968, 95969) c6( ) c7(dP, Vs, T, hB, afc, io, aq, Ld, bu, iR, t, bz, B); #c1(TRO) c2(NP_001258113) c3(trophinin isoform 7) c4(95970, 95971) c5(95972, 95973, 95974, 95975, 95976, 95977, 95978) c6( ) c7(dP, Vs, T, hB, afc, io, aq, Ld, bu, iR, t, bz, B); #c1(TRO) c2(NP_057241) c3(trophinin isoform 2) c4(189688, 189689, 189690) c5(189691, 189692, 189693, 189694, 189695, 189696, 189697, 189698, 189699, 189700) c6( ) c7(dP, Vs, T, hB, afc, io, aq, Ld, bu, iR, t, bz, B); #c1(TRO) c2(NP_808225) c3(trophinin isoform 4) c4(252725) c5(252726, 252727, 252728, 252729, 252730, 252731, 252732, 252733, 252734, 252735, 252736, 252737) c6( ) c7(dP, Vs, T, hB, afc, io, aq, Ld, bu, iR, t, bz, B); #c1(TRO) c2(XP_006724668) c3(trophinin isoform X3) c4(296186, 296187, 296188, 296189) c5(296190, 296191, 296192, 296193, 296194, 296195, 296196, 296197, 296198, 296199, 296200, 296201, 296202, 296203, 296204, 296205, 296206, 296207, 296208, 296209, 296210, 296211, 296212, 296213, 296214, 296215, 296216, 296217, 296218, 296219, 296220, 296221, 296222, 296223, 296224, 296225, 296226, 296227, 296228, 296229, 296230, 296231, 296232, 296233, 296234, 296235, 296236, 296237, 296238, 296239, 296240, 296241, 296242, 296243, 296244, 296245, 296246, 296247, 296248, 296249) c6( ) c7(dP, Vs, T, hB, afc, io, aq, Ld, bu, iR, t, bz, B); #c1(TRO) c2(XP_011529117) c3(trophinin isoform X2) c4(324448, 324449, 324450) c5( ) c6( ) c7(dP, Vs, T, hB, afc, io, aq, Ld, bu, iR, t, bz, B); #c1(TROVE2) c2(NP_001035828) c3(60 kDa SS-A/Ro ribonucleoprotein isoform 1) c4(33182) c5(33183, 33184, 33185, 33186, 33187, 33188) c6( ) c7(bnX, sN, cQn, aW, fN, aT, eZ, fL, mC); #c1(TROVE2) c2(NP_001035829) c3(60 kDa SS-A/Ro ribonucleoprotein isoform 3) c4(33189, 33190) c5(33191, 33192, 33193, 33194, 33195, 33196) c6( ) c7(bnX, sN, cQn, aW, fN, aT, eZ, fL, mC); #c1(TROVE2) c2(NP_001166995) c3(60 kDa SS-A/Ro ribonucleoprotein isoform 2) c4(68783, 68784) c5(68785, 68786, 68787, 68788, 68789, 68790, 68791, 68792, 68793, 68794, 68795, 68796, 68797, 68798, 68799) c6( ) c7(bnX, sN, cQn, aW, fN, aT, eZ, fL, mC); #c1(TROVE2) c2(NP_001166996) c3(60 kDa SS-A/Ro ribonucleoprotein isoform 4) c4(68800) c5(68801, 68802, 68803, 68804, 68805, 68806, 68807, 68808, 68809, 68810, 68811, 68812) c6( ) c7(bnX, sN, cQn, aW, fN, aT, eZ, fL, mC); #c1(TROVE2) c2(XP_011508224) c3(60 kDa SS-A/Ro ribonucleoprotein isoform X3) c4( ) c5(298853, 298854, 298855, 298856, 298857) c6( ) c7(bnX, sN, cQn, aW, fN, aT, eZ, fL, mC); #c1(TRPA1) c2(XP_011515926) c3(transient receptor potential cation channel subfamily A member 1 isoform X2) c4(309075) c5(309076, 309077, 309078, 309079, 309080, 309081) c6( ) c7(bM, T, oe, aw, azd, auq, aj, fu, lN, cQw, V, cd, J, ai, dP, ft, aTT, ji, af, cQv, ES, sp, mQ, zD, bz, cmk, iP, ac, bP, lI, xZ, bTg); #c1(TRPA1) c2(XP_011515927) c3 (transient receptor potential cation channel subfamily A member 1 isoform X1) c4( ) c5(309082, 309083, 309084, 309085, 309086, 309087) c6( ) c7(bM, T, oe, aw, azd, auq, aj, fu, lN, cQw, V, cd, J, ai, dP, ft, aTT, ji, af, cQv, ES, sp, mQ, zD, bz, cmk, iP, ac, bP, lI, xZ, bTg); #c1(TRPC1) c2(NP_001238774) c3(short transient receptor potential channel 1 isoform 1) c4(87900, 87901) c5(87902, 87903, 87904, 87905, 87906, 87907) c6( ) c7(bL, lD, T, eR, cz, bB, qj, ci, vN, ad, eA, BY, aT, K, bq, ae, Zm, bsK, A, aW, Y, Vk, bz, eM, bE, pg, y, pC); #c1(TRPC1) c2(NP_003295) c3(short transient receptor potential channel 1 isoform 2) c4(137211) c5(137212, 137213, 137214, 137215, 137216, 137217, 137218) c6( ) c7(bL, lD, T, eR, cz, bB, qj, ci, vN, ad, eA, BY, aT, K, bq, ae, Zm, bsK, A, aW, Y, Vk, bz, eM, bE, pg, y, pC); #c1(TRPC1) c2(XP_005247796) c3(short transient receptor potential channel 1 isoform X2) c4( ) c5(267996, 267997, 267998, 267999, 268000) c6( ) c7(bL, lD, T, eR, cz, bB, qj, ci, vN, ad, eA, BY, aT, K, bq, ae, Zm, bsk, A, aW, Y, Vk, bz, eM, bE, pg, y, pC); #c1(TRPC3) c2(NP_001124170) c3(short transient receptor potential channel 3 isoform a) c4(48387) c5(48388, 48389, 48390, 48391, 48392, 48393, 48394, 48395, 48396, 48397, 48398, 48399, 48400, 48401, 48402, 48403, 48404, 48405) c6( ) c7(ID, T, mr, cz, j, cd, ad, BY, fU, cF, bk, K, akr, SD, ae, azh, sj, t, qa, alC, ky, m, lC, iu, cQx); #c1(TRPC3) c2(NP_003296) c3(short transient receptor potential channel 3 isoform b) c4(137219, 137220) c5(137221, 137222, 137223, 137224, 137225) c6( ) c7(ID, T, mr, cz, j, cd, ad, BY, fU, cF, bK, K, akr, SD, ae, azh, sj, t, qa, alC, ky, m, lC, iu, cQx); #c1(TRPC3) c2(XP_011530520) c3(short transient receptor potential channel 3 isoform X2) c4( ) c5(326448) c6( ) c7(ID, T, mr, cz, j, cd, ad, BY, fU, cF, bK, K, akr, SD, ae, azh, sj, t, qa, alC, ky, m, lC, iu, cQx); #c1(TRPC3) c2(XP_011530521) c3(short transient receptor potential channel 3 isoform X3) c4(326449) c5( ) c6( ) c7(ID, T, mr, cz, j, cd, ad, BY, FU, cF, bK, K, akr, SD, ae, azh, sj, t, qa, alC, ky, m, lC, iu, cQx); #c1(TRPC4) c2(NP_001129427) c3(short transient receptor potential channel 4 isoform beta) c4(51483) c5(51484, 51485, 51486, 51487, 51488, 51489) c6( ) c7(ac, aw, cD, pg, fr, hg, bX, ir, lC, fq); #c1(TRPC4) c2(NP_001129428) c3(short transient receptor potential channel 4 isoform gamma) c4(51490, 51491) c5(51492, 51493, 51494, 51495, 51496, 51497, 51498, 51499, 51500, 51501, 51502, 51503) c6( ) c7(ac, aw, cD, pg, fr, hg, bX, ir, lC, fq); #c1(TRPC4) c2(NP_001129429) c3(short transient receptor potential channel 4 isoform delta) c4(51504) c5(51505, 51506, 51507, 51508, 51509, 51510, 51511, 51512, 51513, 51514) c6( ) c7(ac, aw, cD, pg, fr, hg, bX, ir, lC, fq); #c1(TRPC4) c2(NP_001129430) c3(short transient receptor potential channel 4 isoform zeta) c4(51515) c5(51516, 51517, 51518, 51519, 51520, 51521, 51522, 51523, 51524, 51525) c6( ) c7(ac, aw, cD, pg, fr, hg, bX, ir, lC, fq); #c1(TRPC4) c2(NP_003297) c3(short transient receptor potential channel 4 isoform epsilon) c4(137226) c5(137227, 137228, 137229, 137230, 137231, 137232) c6( ) c7(ac, aw, cD, pg, fr, hg, bX, ir, lC, fq); #c1(TRPC4) c2(NP_057263) c3(short transient receptor potential channel 4 isoform alpha) c4(189753) c5(189754, 189755, 189756, 189757, 189758, 189759) c6( ) c7(ac, aw, cD, pg, fr, hg, bX, ir, IC, fq); #c1(TRPC4AP) c2(NP_955400) c3(short transient receptor potential channel 4-associated protein isoform b) c4(261710) c5(261711, 26/712, 261713, 261714, 261715, 261716) c6( ) c7(bNX, T, af); #c1(TRPC4AP) c2(XP_011527072) c3(short transient receptor potential channel 4-associated protein isoform X1) c4(321522, 321523, 321524) c5(321525, 321526, 321527, 321528, 321529, 321530, 321531, 321532, 321533, 321534, 321535, 321536, 321537, 321538, 321539, 321540, 321541, 321542) c6( ) c7(bNX, T, af); #c1(TRPC4AP) c2(XP_011527076) c3(short transient receptor potential channel 4-associated protein isoform X5) c4(321543) c5(321544, 321545, 321546, 321547, 321548, 321549) c6( ) c7(bNX, T, af); #c1(TRPC5) c2(NP_036603) c3(short transient receptor potential channel 5) c4(174755) c5(174756, 174757, 174758, 174759, 174760, 174761) c6( ) c7(fU, T, cz, dy, nA, y, hq); #c1(TRPC6) c2(NP_004612) c3(short transient receptor potential channel 6) c4(148158, 148159) c5(148160, 148161, 148162, 148163, 148164, 148165, 148166, 148167, 148168, 148169, 148170, 148171) c6( ) c7(bL, lD, T, eR, dk, e, uv, al, aA, cz, wo, aj, dG, ah, ci, cQy, aY, cd, J, ad, BY, eA, bEk, vw, lG, K, Bz, he, ae, aa, sj, wC, d, ff, gi, cr, vs, aF, en, ir, t, Bx, sk, at, dy, m, lC, uL); #c1(TRPC7) c2(NP_00161048) c3(short transient receptor potential channel 7 isoform 3) c4(65208, 65209) c5(65210, 65211, 65212, 65213, 65214, 65215, 65216, 65217, 65218, 65219, 65220, 65221) c6( ) c7(Y, aj, bK, oi, bw, fq); #c1(TRPC7) c2(NP_001161049) c3(short transient receptor potential channel 7 isoform 2) c4(65222) c5(65223, 65224, 65225, 65226, 65227, 65228) c6( ) c7(Y, aj, bK, oi, bw, fq); #c1(TRPC7) c2(NP_065122) c3(short transient receptor potential channel 7 isoform 1) c4(204053) c5(204054, 204055, 204056, 204057, 204058, 204059) c6( ) c7(Y, aj, bK, oi, bw, fq); #c1(TRPM1) c2(NP_001238949) c3(transient receptor potential cation channel subfamily M member 1 isoform 1) c4(88055, 88056, 88057) c5(88058, 88059, 88060, 88061, 88062, 88063, 88064, 88065) c6( ) c7(Dy, dY, Y, cQA, cQz, ahV, cp, cyN); #c1(TRPM1) c2(NP_001238953) c3(transient receptor potential cation channel subfamily M member 1 isoform 3) c4( ) c5(88066, 88067, 88068, 88069, 88070, 88071) c6( ) c7(Dy, dY, Y, cQA, cQz, ahV, cp, cyN); #c1(TRPM1) c2(NP_001238959) c3(transient receptor potential cation channel subfamily M member 1 isoform 4) c4(88072) c5(88073, 88074, 88075, 88076, 88077, 88078) c6( ) c7(Dy, dY, Y, cQA, cQz, ahV, cp, cyN); #c1(TRPM1) c2(NP_002411) c3(transient receptor potential cation channel subfamily M member 1 isoform 2) c4(128200, 128201, 128202) c5(128203, 128204, 128205, 128206, 128207, 128208, 128209, 128210, 128211, 128212, 128213, 128214, 128215, 128216, 128217, 128218, 128219, 128220, 128221, 128222, 128223, 128224) c6( ) c7(Dy, dY, Y, cQA, cQz, ahV, cp, cyN); #c1(TRPM2) c2(XP_005261228) c3(transient receptor potential cation channel subfamily M member 2 isoform X1) c4(277277) c5(277278, 277279, 277280, 277281, 277282, 277283, 277284, 277285, 277286, 277287) c6( ) c7(bL, T, c, cA, lI, aW, Y, ha, aT, TQ, n, aj, iD, bK, ci, K, lN, oi, J, ad); #c1(TRPM2) c2(XP_006724112) c3(transient receptor potential cation channel subfamily M member 2 isoform X5) c4(295752, 295753) c5(295754, 295755, 295756, 295757, 295758, 295759, 295760, 295761, 295762, 295763, 295764, 295765, 295766, 295767, 295768, 295769, 295770, 295771) c6( ) c7(bL, T, c, cA, lI, aW, Y, ha, aT, TQ, n, aj, iD, bK, ci, K, lN, oi, J, ad); #c1(TRPM2) c2(XP_011528037) c3(transient receptor potential cation channel subfamily M member 2 isoform X2) c4( ) c5(323137) c6( ) c7(bL, T, c, cA, lI, aW, Y, ha, aT, TQ, n, aj, iD, bK, ci, K, lN, oi, J, ad); #c1(TRPM2) c2(XP_011528038) c3(transient receptor potential cation channel subfamily M member 2 isoform X3) c4(323138) c5( ) c6( ) c7(bL, T, c, cA, lI, aW, Y, ha, aT, TO, n, aj, iD, bK, ci, K, lN, oi, J, ad); #c1(TRPM3) c2(NP_001007472) c3(transient receptor potential cation channel subfamily M member 3 isoform k) c4(22742) c5(22743, 22744, 22745, 22746, 22747, 22748) c6( ) c7(cc, py, ac, dY, aw, j, aXL, xa); #c1(TRPM3) c2(NP_066003) c3 (transient receptor potential cation channel subfamily M member 3 isoform a) c4(206715, 206716) c5( ) c6( ) c7(cc, py, ac, dY, aw, j, aXL, xa); #c1(TRPM3) c2(NP_079247) c3(transient receptor potential cation channel subfamily M member 3 isoform b) c4(217592) c5( ) c6( ) c7(cc, py, ac, dY, aw, j, aXL, xa); #c1(TRPM3) c2(NP_996827) c3(transient receptor potential cation channel subfamily M member 3 isoform d) c4(264672) c5( ) c6( ) c7(cc, py, ac, dY, aw, j, aXL, xa); #c1(TRPM3) c2(NP_996828) c3(transient receptor potential cation channel subfamily M member 3 isoform e) c4(264673) c5( ) c6( ) c7(cc, py, ac, dY, aw, j, aXL, xa); #c1(TRPM3) c2(NP_996829) c3(transient receptor potential cation channel subfamily M member 3 isoform f) c4(264674) c5( ) c6( ) c7(cc, py, ac, dY, aw, j, aXL, xa); #c1(TRPM3) c2(NP_996830) c3(transient receptor potential cation channel subfamily M member 3 isoform g) c4(264675) c5( ) c6( ) c7(cc, py, ac, dY, aw, j, aXL, xa); #c1(TRPM3) c2(NP_996831) c3 (transient receptor potential cation channel subfamily M member 3 isoform h) c4( ) c5(264676, 264677, 264678, 264679, 264680, 264681, 264682, 264683, 264684, 264685) c6( ) c7(cc, py, ac, dY, aw, j, aXL, xa); #c1(TRPM3) c2(XP_011517337) c3(transient receptor potential cation channel subfamily M member 3 isoform X1) c4(311018) c5(311019, 311020, 311021, 311022, 311023, 311024, 311025) c6( ) c7(cc, py, ac, dY, aw, j, aXL, xa); #c1(TRPM3) c2(XP_011517340) c3(transient receptor potential cation channel subfamily M member 3 isoform X4) c4( ) c5(311026, 311027, 311028, 311029, 311030, 311031, 311032, 311033, 311034, 311035) c6( ) c7(cc, py, ac, dY, aw, j, aXL, xa); #c1(TRPM3) c2(XP_011517341) c3(transient receptor potential cation channel subfamily M member 3 isoform X5) c4( ) c5(311036, 311037, 311038, 311039, 311040, 311041, 311042, 311043, 311044, 311045) c6( ) c7(cc, py, ac, dY, aw, j, aXL, xa); #c1(TRPM3) c2(XP_011517343) c3(transient receptor potential cation channel subfamily M member 3 isoform X7) c4( ) c5(311046, 311047, 311048, 311049, 311050, 311051, 311052, 311053, 311054, 311055) c6( ) c7(cc, py, ac, dY, aw, j, aXL, xa); #c1(TRPM3) c2(XP_011517344) c3(transient receptor potential cation channel subfamily M member 3 isoform X8) c4(311056) c5(311057, 311058, 311059, 311060, 311061, 311062) c6( ) c7(cc, py, ac, dY, aw, j, aXL, xa); #c1(TRPM3) c2(XP_011517345) c3(transient receptor potential cation channel subfamily M member 3 isoform X9) c4( ) c5(311063, 311064, 311065, 311066, 311067, 311068) c6( ) c7(cc, py, ac, dY, aw, j, aXL, xa); #c1(TRPM3) c2(XP_011517346) c3(transient receptor potential cation channel subfamily M member 3 isoform X10) c4( ) c5(311069, 311070, 311071, 311072, 311073, 311074) c6( ) c7(cc, py, ac, dY, aw, j, aXL, xa); #c1(TRPM3) c2(XP_011517349) c3(transient receptor potential cation channel subfamily M member 3 isoform X13) c4(311075) c5(311076, 311077, 311078, 311079, 311080, 311081, 311082, 311083) c6( ) c7(cc, py, ac, dY, aw, j, aXL, xa); #c1(TRPM4) c2(NP_001182156) c3(transient receptor potential cation channel subfamily M member 4 isoform 2) c4(74624) c5(74625, 74626, 74627, 74628, 74629, 74630) c6( ) c7(ae, ajM, eo, K, C, bUw, V, cQB, ad); #c1(TRPM4) c2(NP_060106) c3(transient receptor potential cation channel subfamily M member 4 isoform 1) c4(194182) c5(194183, 194184, 194185, 194186, 194187, 194188) c6( ) c7(ae, ajM, eo, K, C, bUw, V, cQB, ad); #c1(TRPM4) c2(XP_005259074) c3(transient receptor potential cation channel subfamily M member 4 isoform X2) c4(275956) c5( ) c6( ) c7(ae, ajM, eo, K, C, bUw, V, cQB, ad); #c1(TRPM4) c2(XP_005259075) c3 (transient receptor potential cation channel subfamily M member 4 isoform X3) c4( ) c5(275957) c6( ) c7(ae, ajM, eo, K, C, bUw, V, cQB, ad); #c1(TRPM4) c2(XP_011525348) c3(transient receptor potential cation channel subfamily M member 4 isoform X1) c4( ) c5(319812, 319813, 319814, 319815, 319816, 319817) c6( ) c7(ae, ajM, eo, K, C, bUw, V, cQB, ad); #c1(TRPM5) c2(NP_055370) c3(transient receptor potential cation channel subfamily M member 5) c4(180637, 180638, 180639) c5(180640, 180641, 180642, 180643, 180644, 180645, 180646, 180647, 180648, 180649, 180650, 180651, 180652, 180653, 180654, 180655, 180656, 180657, 180658, 180659) c6( ) c7(bL, aW, bv, g, aT); #c1(TRPM5) c2(XP_011518337) c3(transient receptor potential cation channel subfamily M member 5 isoform X1) c4(312337) c5(312338, 312339, 312340, 312341, 312342, 312343) c6( ) c7(bL, aW, bv, g, aT); #c1(TRPM6) c2(NP_001170781) c3(transient receptor potential cation channel subfamily M member 6 isoform b) c4(69512) c5(69513, 69514, 69515, 69516, 69517, 69518) c6( ) c7(bL, TI, uv, cz, sH, aj, Bx, J); #c1(TRPM6) c2(NP_001170782) c3(transient receptor potential cation channel subfamily M member 6 isoform c) c4(69519) c5(69520, 69521, 69522, 69523, 69524, 69525) c6( ) c7(bL, TI, uv, cz, sH, aj, Bx, J); #c1(TRPM6) c2(NP_060132) c3(transient receptor potential cation channel subfamily M member 6 isoform a) c4(194326, 194327, 194328) c5(194329, 194330, 194331, 194332, 194333, 194334, 194335, 194336, 194337, 194338, 194339, 194340, 194341, 194342, 194343, 194344, 194345, 194346) c6( ) c7(bL, TI, uv, cz, sH, aj, Bx, J); #c1(TRPM6) c2(XP_011516550) c3(transient receptor potential cation channel subfamily M member 6 isoform X5) c4(309953) c5( ) c6( ) c7(bL, TI, uv, cz, sH, aj, Bx, J); #c1(TRPM6) c2(XP_011516551) c3(transient receptor potential cation channel subfamily M member 6 isoform X6) c4( ) c5(309954, 309955, 309956, 309957, 309958, 309959) c6( ) c7(bL, TI, uv, cz, sH, aj, Bx, J); #c1(TRPM6) c2(XP_011516552) c3(transient receptor potential cation channel subfamily M member 6 isoform X7) c4(309960) c5( ) c6( ) c7(bL, TI, uv, cz, sH, aj, Bx, J); #c1(TRPM6) c2(XP_011516553) c3(transient receptor potential cation channel subfamily M member 6 isoform X8) c4(309961, 309962, 309963) c5( ) c6( ) c7(bL, TI, uv, cz, sH, aj, Bx, J); #c1(TRPM6) c2(XP_011516554) c3(transient receptor potential cation channel subfamily M member 6 isoform X9) c4(309964) c5(309965, 309966, 309967, 309968, 309969, 309970, 309971, 309972, 309973, 309974, 309975, 309976) c6( ) c7(bL, TI, uv, cz, sH, aj, Bx, J); #c1(TRPM6) c2(XP_011516555) c3(transient receptor potential cation channel subfamily M member 6 isoform X10) c4( ) c5(309977, 309978, 309979, 309980, 309981, 309982) c6( ) c7(bL, TI, uv, cz, sH, aj, Bx, J); #c1(TRPM6) c2(XP_011516557) c3(transient receptor potential cation channel subfamily M member 6 isoform X12) c4( ) c5(309983, 309984, 309985, 309986, 309987, 309988, 309989, 309990, 309991, 309992, 309993, 309994) c6( ) c7(bL, TI, uv, cz, sH, aj, Bx, J); #c1(TRPM7) c2(NP_060142) c3 (transient receptor potential cation channel subfamily M member 7 isoform a) c4(194420) c5(194421, 194422, 194423, 194424, 194425, 194426) c6( ) c7(bL, T, mr, byf, e, al, aA, cz, aj, dG, ci, eJ, J, M, aT, lG, cy, K, an, eu, s, Jl, aW, Y, aav, aF, mo, Bx, ac, c, U, aS); #c1(TRPM8) c2(NP_076985) c3(transient receptor potential cation channel subfamily M member 8) c4(214601, 214602, 214603, 214604) c5(214605, 214606, 214607, 214608, 214609, 214610, 214611, 214612, 214613, 214614, 214615, 214616, 214617, 214618, 214619, 214620, 214621, 214622, 214623, 214624, 214625, 214626, 214627) c6( ) c7(gh, cZ, T, bM, auq, v, ur, uu, q, ad, crU, pX, aT, dd, ae, aW, xi, Y, eH, ff, r, kp, en, g, eF, vo, cm, bz, jb, Jp); #c1(TRPM8) c2(XP_011510112) c3 (transient receptor potential cation channel subfamily M member 8 isoform X1) c4( ) c5(300710, 300711) c6( ) c7(gh, cZ, T, bM, auq, v, ur, uu, q, ad, crU, pX, aT, dd, ae, aW, xi, Y, eH, ff, r, kp, en, g, eF, vo, cm, bz, jb, Jp); #c1(TRPS1) c2(NP_001269831) c3(zinc finger transcription factor Trps1 isoform 2) c4(102920) c5(102921, 102922, 102923, 102924, 102925, 102926) c6( ) c7(rV, oe, cz, asM, ci, bX, zP, ad, cF, boY, Db, aT, ae, bdw, Dg, zQ, TY, aW, cQD, Az, eH, cQC, aSt, mv, eF, dr, EW, ac, auZ, gZ, y, bt); #c1(TRPS1) c2(XP_011515568) c3(zinc finger transcription factor Trps1 isoform X2) c4(308405) c5(308406, 308407, 308408, 308409, 308410, 308411, 308412) c6( ) c7(rV, oe, cz, asM, ci, bX, zP, ad, cF, boY, Db, aT, ae, bdw, Dg, zl, TY, aW, cQD, Az, eH, cQC, aSt, mv, eF, dr, EW, ac, auZ, gZ, y, bt); #c1(TRPS1) c2(XP_011515569) c3(zinc finger transcription factor Trps1 isoform X1) c4(308413) c5(308414) c6( ) c7(rV, oe, cz, asM, ci, bX, zP, ad, cF, boY, Db, aT, ae, bdw, Dg, zQ, TY, aW, cQD, Az, eH, cQC, aSt, mv, eF, dr, EW, ac, auZ, gZ, y, bt); #c1(TRPS1) c2(XP_011515570) c3(zinc finger transcription factor Trps1 isoform X3) c4( ) c5(308415, 308416, 308417, 308418, 308419) c6( ) c7(rV, De, cz, asM, ci, bX, zP, ad, cF, boY, Db, aT, ae, bdw, Dg, zQ, TY, aW, cQD, Az, eH, cQC, aSt, mv, eF, dr, EW, ac, auZ, gZ, y, bt); #c1(TRPV1) c2(NP_542437) c3(transient receptor potential cation channel subfamily V member 1) c4(232940, 232941, 232942, 232943) c5(232944, 232945, 232946, 232947, 232948, 232949, 232950, 232951, 232952, 232953, 232954, 232955, 232956, 232957, 232958, 232959, 232960, 232961, 232962, 232963, 232964, 232965, 232966, 232967, 232968, 232969, 232970, 232971, 232972, 232973, 232974, 232975, 232976, 232977, 232978, 232979) c6( ) c7(bM, cZ, T, dD, akd, e, uv, azd, alH, ur, cQH, uu, pM, dG, aj, v, eq, ai, J, ad, aAq, cQF, dP, eA, uT, Jb, pX, cE, h, cy, dL, K, UN, dd, aNR, aDW, ae, eL, bEV, DI, aFs, tn, d, mQ, fc, fQ, aiL, cQE, akY, gu, coP, as, iV, cm, jb, DF, bN, ar, D, Ta, iI, bP, iO, xZ, uR, at, cQG, ra, zB, bFC, y, Tc); #c1(TRPV2) c2(NP_057197) c3(transient receptor potential cation channel subfamily V member 2) c4(189449) c5(189450, 189451, 189452, 189453, 189454, 189455) c6( ) c7(T, cc, d, id, lI, Ae, eL, e, xs, fd, dL, lN, bgq, ae, eA, ad); #c1(TRPV2) c2(XP_006721606) c3 (transient receptor potential cation channel subfamily V member 2 isoform X4) c4(294118) c5( ) c6( ) c7(T, cc, d, id, lI, Ae, eL, e, xs, fd, dL, lN, bgq, ae, eA, ad); #c1(TRPV2) c2(XP_011522226) c3(transient receptor potential cation channel subfamily V member 2 isoform X8) c4( ) c5(316850, 316851, 316852, 316853, 316854, 316855) c6( ) c7(T, cc, d, id, lI, Ae, eL, e, xs, fd, dL, lN, bgq, ae, eA, ad); #c1(TRPV3) c2(NP_001245134) c3(transient receptor potential cation channel subfamily V member 3 isoform 1) c4(92539, 92540) c5(92541, 92542, 92543, 92544, 92545, 92546, 92547, 92548, 92549, 92550, 92551, 92552) c6( ) c7(cZ, cQl, ccA, dN, ur, lQ, uu, dd, eE); #c1(TRPV3) c2(NP_659505) c3(transient receptor potential cation channel subfamily V member 3 isoform 2) c4(240270) c5(240271, 240272, 240273, 240274, 240275, 240276, 240277, 240278, 240279, 240280) c6( ) c7(cZ, cQl, ccA, dN, ur, lQ, uu, dd, eE); #c1(TRPV4) c2(NP_001170902) c3(transient receptor potential cation channel subfamily V member 4 isoform d) c4(69591) c5(69592, 69593, 69594, 69595, 69596, 69597) c6( ) c7(bM, Pt, bBr, cV, cQO, fu, bX, pM, Do, fw, aR, zw, no, cQN, CEK, Jb, rw, Ak, kf, cQJ, UN, K, bU, bV, rv, cQQ, cQM, bA, azh, Og, ao, bT, cQS, zD, cQL, fe, cnP, iO, cQR, ql, xZ, cQP, cQK, U, MM, at, wB, bk, aSk, jr); #c1(TRPV4) c2(XP_005253975) c3 (transient receptor potential cation channel subfamily V member 4 isoform X2) c4(272839) c5(272840, 272841, 272842, 272843, 272844, 272845, 272846, 272847, 272848, 272849) c6( ) c7(bM, Pt, bBr, cV, cQO, fu, bX, pM, Qo, fw, aR, zw, no, cQN, CEK, Jb, rw, Ak, kf, cQJ, UN, K, bU, bV, rv, cQQ, cQM, bA, azh, Oq, ao, bT, cQS, zD, cQL, fe, cnP, iD, cQR, ql, xZ, cQP, cQK, U, MM, at, wB, bk, aSk, jr); #c1(TRPV4) c2(XP_011536933) c3(transient receptor potential cation channel subfamily V member 4 isoform X3) c4(334603) c5(334604, 334605, 334606, 334607, 334608, 334609, 334610, 334611, 334612, 334613) c6( ) c7(bM, Pt, bBr, cV, cQO, fu, bX, pM, Qo, fw, aR, zw, no, cQN, cEK, Jb, rw, Ak, kf, cQJ, UN, K, bU, bV, rv, cQQ, cQM, bA, azh, Oq, ao, bT, cQS, zD, cQL, fe, cnP, iO, cQR, ql, xZ, cQP, cQK, U, MM, at, wB, bk, aSk, jr); #c1(TRPV4) c2(XP_011536934) c3(transient receptor potential cation channel subfamily V member 4 isoform X1) c4(334614) c5(334615, 334616, 334617, 334618, 334619, 334620, 334621, 334622, 334623, 334624) c6( ) c7(bM, Pt, bBr, cV, cOQ, fu, bX, pM, Do, fw, aR, zw, no, cQN, cEK, Jb, rw, Ak, kf, cQJ, UN, K, bU, bV, rv, cQQ, cQM, bA, azh, Oq, ao, bT, cQS, zD, cQL, fe, cnP, iO, cQR, ql, xZ, cQP, cQK, U, MM, at, wB, bk, aSk, jr); #c1(TRPV4) c2(XP_011536935) c3(transient receptor potential cation channel subfamily V member 4 isoform X4) c4(334625) c5(334626, 334627, 334628, 334629, 334630, 334631) c6( ) c7(bM, Pt, bBr, cV, cQO, fu, bX, pM, Do, fw, aR, zw, no, cON, cEK, Jb, rw, Ak, kf, cQJ, UN, K, bU, bV, rv, cQQ, cQM, bA, azh, Oq, ao, bT, cQS, zD, cQL, fe, cnP, iO, cQR, ql, xZ, cQP, cQK, U, MM, at, wB, bk, aSk, jr); #c1(TRPV5) c2(NP_062815) c3(transient receptor potential cation channel subfamily V member 5) c4(202089, 202090) c5(202091, 202092, 202093, 202094, 202095, 202096, 202097, 202098, 202099, 202100, 202101, 202102) c6( ) c7(ck, TI, iT, ig, aAP, aoo, cz, aj, afV, bWB, K, C, ars, J); #c1(TRPV6) c2(NP_061116) c3(transient receptor potential cation channel subfamily V member 6) c4(199179, 199180) c5(199181, 199182, 199183, 199184, 199185, 199186, 199187, 199188, 199189, 199190, 199191, 199192) c6( ) c7(T, aYJ, if, jh, bB, Fb, ci, nA, cd, q, ad, aT, Ko, K, lQ, bK, an, UK, ae, azh, kl, aW, r, g, qa, dr, ck, alC, ig, bE, m, im); #c1(TRRAP) c2(NP_001231509) c3 (transformation/transcription domain-associated protein isoform 1) c4(87267) c5(87268, 87269, 87270, 87271, 87272, 87273, 87274, 87275, 87276) c6( ) c7(BI, T, Y, eD, rU, U, jh, rO, bUo, ae, ad); #c1(TRRAP) c2(NP_003487) c3(transformation/transcription domain-associated protein isoform 2) c4(138726, 138727) c5(138728, 138729, 138730, 138731, 138732, 138733, 138734, 138735, 138736, 138737, 138738, 138739) c6( ) c7(BI, T, Y, eD, rU, U, jh, rO, bUo, ae, ad); #c1(TSACC) c2(NP_001291755) c3(TSSK6-activating co-chaperone protein isoform c) c4(116478) c5(116479, (16480, 116481, 116482, 116483) c6( ) c7(cc, aE, iA, aW, aA, aT, aF, fr, eA); #c1(TSACC) c2(NP_653228) c3(TSSK6-activating co-chaperone protein isoform a) c4( ) c5(239206, 239207, 239208, 239209, 239210, 239211, 239212, 239213, 239214, 239215, 239216, 239217, 239218, 239219, 239220, 239221, 239222) c6( ) c7(cc, aE, iA, aW, aA, aT, aF, fr, eA); #c1(TSC1) c2(NP_001155899) c3(hamartin isoform 4) c4(61632, 61633, 61634) c5(61635, 61636, 61637, 61638, 61639, 61640) c6( ) c7(T, jW, Js, hV, ea, if, pf, al, Xi, lr, fd, bB, ci, aY, ip, x, bv, aE, Xc, iI, cQT, pX, aeO, Qk, avA, ajv, iv, gs, K, aDZ, hu, bOZ, pk, eL, aa, auQ, yo, aeR, Jl, aW, Ls, CT, dg, g, as, eF, zE, ir, cm, apB, bw, iP, qJ, eM, nB, iA, lM, iT, go, wB, cj, iS, QF, cX, azg, m, in, ik, iu, sc, asW, dD, lT, ht, cQU, Bm, n, aj, fr, nA, qN, br, Mo, ft, jz, Do, gd, h, yZ, afV, dL, is, Xh, pi, aeJ, cC, Lg, Dg, asp, CN, iw, alt, ks, aq, agF, kp, pm, alf, ou, iO, byl, YE, bP, ky, dB, hk, Vy, hL, hR, pg, HB, MW, SZ, avr, YH, anx, Az, it, B, Jo, aAE, bL, fM, yk, jx, fZ, aA, cz, hy, bol, yO, Qo, yU, lu, q, J, er, eA, XX, oT, mj, RI, lg, nv, hb, qK, aT, cxs, dz, zp, jj, rz, ae, ne, jf, qw, Uf, Yw, s, no, iU, Y, ev, Lf, r, baP, lo, eS, Lc, en, bu, cl, t, ho, iK, bN, ck, sS, nM, kP, Dy, QG, gX, aJI, dy, zu, bt, VB, hS, Mb, hl, kh, id, pY, ha, mO, jh, KO, oZ, Fb, Gn, jE, eq, adg, hF, Fc, anu, xt, ad, hX, Jm, dP, av, hd, ib, py, iC, bjn, Db, bkq, NS, arV, io, HD, bj, hP, ji, nJ, aAL, d, BN, aQp, bug, BO, TY, vj, et, qt, eH, cr, ff, Ej, Xj, aF, iR, bz, dr, qa, bF, es, gk, Ew, bE, afd, aS, bC, MC, buL, KS, X); #c1(TSC1) c2(XP_006717335) c3(hamartin isoform X2) c4( ) c5(290921, 290922, 290923, 290924, 290925, 290926, 290927, 290928, 290929, 290930, 290931, 290932, 290933, 290934, 290935, 290936, 290937, 290938) c6( ) c7(T, jW, Js, hV, ea, if, pf, al, Xi, lr, fd, bB, ci, aY, ip, x, bv, aE, Xc, iI, cQT, pX, aeO, Qk, avA, ajv, iv, gs, K, aDZ, hu, bOZ, pk, eL, aa, auQ, yo, aeR, Jl, aW, Ls, CT, dg, g, as, eF, zE, ir, cm, apB, bw, iP, qJ, eM, nB, iA, IM, iT, go, wB, cj, iS, QF, cX, azg, m, in, ik, iu, sc, asW, dD, lT, ht, cQU, Bm, n, aj, fr, nA, qN, br, Mo, ft, jz, Do, gd, h, yZ, afV, dL, is, Xh, pi, aeJ, cC, Lg, Dg, asp, CN, iw, alt, ks, aq, agF, kp, pm, alf, ou, iO, byl, YE, bP, ky, dB, hk, Vy, hL, hR, pg, HB, MW, SZ, avr, YH, anx, Qz, it, B, Jo, aAE, bL, fM, yk, jx, fZ, aA, cz, hy, bol, yO, Qo, yU, lu, q, J, er, eA, XX, OT, mj, RI, lg, nv, hb, qK, aT, cxs, dz, zp, jj, rz, ae, ne, jf, qw, Uf, Yw, s, nO, iU, Y, ev, Lf, r, baP, lo, eS, Lc, en, bu, cl, t, ho, iK, bN, ck, sS, nM, kP, Dy, QG, gX, aJQ, dy, zu, bt, VB, hS, Mb, hl, kh, id, pY, ha, mO, jh, KO, oZ, Fb, Gn, jE, eq, adg, hF, Fc, anu, xt, ad, hX, Jm, dP, av, hd, ib, py, iC, bjn, Db, bkq, NS, arV, io, HD, bj, hP, ji, nJ, aAL, d, BN, alp, bug, BO, TY, vj, et, qt, eH, cr, ff, Ej, Xj, aF, iR, bz, dr, qa, bF, es, gk, Ew, bE, afd, aS, bC, MC, buL, KS, X); #c1(TSC1) c2(XP_011517281) c3(hamartin isoform X1) c4(310870, 310871) c5(310872, 310873, 310874, 310875, 310876, 310877, 310878, 310879, 310880, 310881) c6( ) c7(T, jW, Js, hV, ea, if, pf, al, Xi, lr, fd, bB, ci, aY, ip, x, bv, aE, Xc, iI, cQT, pX, aeO, Qk, avA, ajv, iv, gs, K, aDZ, hu, bOZ, pk, eL, aa, auQ, yo, aeR, Jl, aW, Ls, CT, dg, g, as, eF, zE, ir, cm, apB, bw, iP, qJ, eM, nB, iA, lM, iT, go, wB, cj, iS, QF, cX, azg, m, in, ik, iu, sc, asW, dD, lT, ht, cQU, Bm, n, aj, fr, nA, qN, br, Mo, ft, jz, Do, gd, h, yZ, afV, dL, is, Xh, pi, aeJ, cC, Lg, Dg, asp, CN, iw, alt, ks, aq, agF, kp, pm, alf, oU, iO, byl, YE, bP, ky, dB, hk, Vy, hL, hR, pg, HB, MW, SZ, avr, YH, anx, Qz, it, B, Jo, aAE, bL, fM, yk, jx, fZ, aA, cz, hy, bol, yO, Qo, yU, lu, q, J, er, eA, XX, oT, mj, RI, lg, nv, hb, qK, aT, cxs, dz, zp, jj, rz, ae, ne, jf, qw, Uf, Yw, s, nO, iU, Y, ev, Lf, r, baP, lo, eS, Lc, en, bu, cl, t, ho, iK, bN, ck, sS, nM, kP, Dy, QG, gX, aJQ, dy, zu, bt, VB, hS, Mb, hl, kh, id, pY, ha, mO, jh, KO, oZ, Fb, Gn, jE, eq, adg, hF, Fc, anu, xt, ad, hX, Jm, dP, av, hd, ib, py, iC, bjn, Db, bkq, NS, arV, io, hD, bj, hP, ji, nJ, aAL, d, BN, alp, bug, BO, TY, vj, et, qt, eH, cr, ff, Ej, Xj, af, iR, bz, dr, qa, bF, es, gk, Ew, bE, afd, aS, bC, MC, buL, kS, X); #c1(TSC2) c2(NP_000539) c3(tuberin isoform 1) c4(12398) c5(12399, 12400, 12401, 12402, 12403, 12404) c6( ) c7(T, aC, pf, Xi, lr, fd, ci, aY, aR, K, Nj, bOZ, MO, pk, aa, yo, aW, g, as, Jl, iA, go, vA, wB, vF, ik, cE, bSp, cQU, n, aj, fr, PY, nA, Mo, cty, KN, h, dL, Xh, Lg, cC, awV, alt, op, hq, bP, dB, eD, hL, cB, eA, J, lg, hb, HC, aT, an, rz, af, jf, bf, s, Y, en, bN, dy, bt, hl, id, arS, ha, jE, hF, anu, bKb, xt, hX, Jm, iC, bkq, ib, Db, NS, arV, ff, Xj, Z, bz, dr, es, MC, lM, X); #c1(TSC2) c2(NP_001107854) c3(tuberin isoform 5) c4(43033) c5(43034, 43035, 43036, 43037, 43038, 43039) c6( ) c7(T, aC, pf, Xi, lr, fd, ci, aY, aR, K, Nj, bOZ, MO, pk, aa, yo, aW, g, as, Jl, iA, go, vA, wB, vF, ik, cE, bSp, cQU, n, aj, fr, PY, nA, Mo, cty, KN, h, dL, Xh, Lg, cC, awV, alt, op, hq, bP, dB, eD, hL, cB, eA, J, lg, hb, HC, aT, an, rz, af, jf, bf, s, Y, en, bN, dy, bt, hl, id, arS, ha, jE, hF, anu, bKb, xt, hX, Jm, iC, bkq, ib, Db, NS, arV, ff, Xj, Z, bz, dr, es, MC, lM, X); #c1(TSC2) c2(XP_005255586) c3(tuberin isoform X6) c4(273737) c5(273738, 273739, 273740, 273741, 273742, 273743) c6( ) c7(T, aC, pf, Xi, lr, fd, ci, aY, aR, K, Nj, bOZ, MO, pk, aa, yo, aW, g, as, Jl, iA, go, vA, wB, vF, ik, cE, bSp, cQU, n, aj, fr, PY, nA, Mo, cty, KN, h, dL, Xh, Lg, cC, awV, alt, op, hq, bP, dB, eD, hL, cB, eA, J, lg, hb, HC, aT, an, rz, af, jf, bf, s, Y, en, bN, dy, bt, hl, id, arS, ha, jE, hF, anu, bKb, xt, hX, Jm, iC, bkq, ib, Db, NS, arV, ff, Xj, Z, bz, dr, es, MC, lM, X); #c1(TSC2) c2(XP_005255588) c3(tuberin isoform X7) c4(273744, 273745) c5(273746, 273747, 273748, 273749, 273750, 273751, 273752, 273753, 273754, 273755, 273756, 273757) c6( ) c7(T, aC, pf, Xi, lr, fd, ci, aY, aR, K, Nj, bOZ, MO, pk, aa, yo, aW, g, as, Jl, iA, go, vA, wB, vF, ik, cE, bSp, cQU, n, aj, fr, PY, nA, Mo, cty, KN, h, dL, Xh, Lg, cC, awV, alt, op, hq, bP, dB, eD, hL, cB, eA, J, lg, hb, HC, aT, an, rz, af, jf, bf, s, Y, en, bN, dy, bt, hl, id, arS, ha, jE, hF, anu, bKb, xt, hX, Jm, iC, bkq, ib, Db, NS, arV, ff, Xj, Z, bz, dr, es, MC, lM, X); #c1(TSC2) c2(XP_011520943) c3(tuberin isoform X8) c4(315943) c5(315944, 315945, 315946, 315947, 315948, 315949) c6( ) c7(T, aC, pf, Xi, lr, fd, ci, aY, aR, K, Nj, bOZ, MO, pk, aa, yo, aW, g, as, Jl, iA, go, vA, wB, vF, ik, cE, bSp, cQU, n, aj, fr, PY, nA, Mo, cty, KN, h, dL, Xh, Lg, cC, awV, alt, op, hq, bP, dB, eD, hL, cB, eA, J, lg, hb, HC, aT, an, rz, af, jf, bf, s, Y, en, bN, dy, bt, hl, id, arS, ha, jE, hF, anu, bKb, xt, hX, Jm, iC, bkq, ib, Db, NS, arV, ff, Xj, Z, bz, dr, es, MC, lM, X); #c1(TSC22D1) c2(NP_001230728) c3(TSC22 domain family protein 1 isoform 4) c4(86726, 86727) c5(86728, 86729, 86730, 86731, 86732, 86733, 86734, 86735, 86736, 86737, 86738, 86739) c6( ) c7(bL, T, zQ, jp, dB, if, qy, aT, jh, QE, Qb, ci, qN, as, zP, Fc, ae, amW, ad); #c1(TSC22D1) c2(NP_006013) c3(TSC22 domain family protein 1 isoform 2) c4(161151, 161152) c5(161153, 161154, 161155, 161156, 161157, 161158, 161159, 161160, 161161, 161162, 161163, 161164, 161165) c6( ) c7(bL, T, zQ, jp, dB, if, qy, aT, jh, QE, Qb, ci, qN, as, zP, Fc, ae, amW, ad); #c1(TSC22D1) c2(NP_904358) c3(TSC22 domain family protein 1 isoform 1) c4(258096) c5(258097, 258098, 258099, 258100, 258101, 258102) c6( ) c7(bL, T, zQ, jp, dB, if, qy, aT, jh, QE, Qb, ci, qN, as, zP, Fc, ae, amW, ad); #c1(TSC22D3) c2(NP_001015881) c3(TSC22 domain family protein 3 isoform 3) c4( ) c5(26228, 26229, 26230, 26231, 26232, 26233, 26234, 26235, 26236, 26237) c6( ) c7(ck, bM, gU, xE, rq, byn, Y, Ei, fd, jy, rs, v, ci, Ca, brw, ai, y, bA); #c1(TSC2203) c2(NP_004080) c3(TSC22 domain family protein 3 isoform 2) c4(143381, 143382, 143383, 143384) c5(143385, 143386, 143387, 143388, 143389, 143390, 143391, 143392, 143393, 143394, 143395, 143396, 143397, 143398, 143399) c6( ) c7(ck, bM, gU, xE, rq, byn, Y, Ei, fd, jy, rs, v, ci, Ca, brw, ai, y, bA); #c1(TSC22D3) c2(NP_932174) c3(TSC22 domain family protein 3 isoform 1) c4(258863, 258864, 258865, 258866, 258867, 258868, 258869, 258870, 258871, 258872) c5(258873, 258874, 258875, 258876, 258877, 258878, 258879, 258880, 258881, 258882, 258883, 258884, 258885, 258886, 258887, 258888, 258889, 258890, 258891, 258892, 258893, 258894, 258895, 258896, 258897, 258898, 258899, 258900, 258901, 258902, 258903, 258904, 258905, 258906, 258907, 258908, 258909, 258910, 258911, 258912, 258913, 258914, 258915, 258916, 258917, 258918, 258919, 258920, 258921, 258922, 258923, 258924, 258925, 258926, 258927, 258928, 258929, 258930, 258931, 258932, 258933, 258934, 258935, 258936, 258937, 258938, 258939, 258940, 258941, 258942, 258943, 258944) c6( ) c7(ck, bM, gU, xE, rq, byn, Y, Ei, fd, jy, rs, v, ci, Ca, brw, ai, y, bA); #c1(TSC2204) c2(NP_001289972) c3(TSC22 domain family protein 4 isoform a) c4(114886) c5(114887, 114888, 114889, 114890, 114891, 114892, 114893) c6( ) c7(amk, T); #c1(TSEN2) c2(NP_001138865) c3(tRNA-splicing endonuclease subunit Sen2 isoform 2) c4(57938) c5(57939, 57940, 57941, 57942, 57943, 57944, 57945, 57946, 57947, 57948) c6( ) c7(QK, xw, cQV, ad, nA); #c1(TSEN2) c2(NP_001138866) c3(tRNA-splicing endonuclease subunit Sen2 isoform 3) c4(57949) c5(57950, 57951, 57952, 57953, 57954, 57955) c6( ) c7(QK, xw, cQV, ad, nA); #c1(TSEN2) c2(NP_001138867) c3(tRNA-splicing endonuclease subunit Sen2 isoform 4) c4(57956) c5(57957, 57958, 57959, 57960, 57961, 57962) c6( ) c7(QK, xw, cQV, ad, nA); #c1(TSEN2) c2(NP_079541) c3(tRNA-splicing endonuclease subunit Sen2 isoform 1) c4(219161, 219162, 219163, 219164) c5(219165, 219166, 219167, 219168, 219169, 219170, 219171, 219172, 219173, 219174, 219175, 219176, 219177, 219178, 219179, 219180, 219181, 219182, 219183, 219184, 219185, 219186, 219187, 219188, 219189, 219190, 219191, 219192) c6( ) c7(QK, xw, cQV, ad, nA); #c1(TSEN34) c2(NP_001269261) c3(tRNA-splicing endonuclease subunit Sen34 isoform 1) c4( ) c5(100933, 100934, 100935, 100936, 100937, 100938, 100939, 100940, 100941, 100942, 100943, 100944, 100945, 100946, 100947, 100948, 100949, 100950, 100951, 100952, 100953, 100954, 100955, 100956, 100957, 100958, 100959, 100960, 100961, 100962, 100963, 100964, 100965, 100966, 100967, 100968, 100969, 100970, 100971, 100972, 100973, 100974, 100975, 100976, 100977, 100978, 100979, 100980, 100981, 100982, 100983, 100984, 100985) c6( ) c7(UR, QK, xw, cQV, nA); #c1(TSEN34) c2(NP_001269262) c3(tRNA-splicing endonuclease subunit Sen34 isoform 2) c4(100986, 100987) c5(100988, 100989, 100990, 100991, 100992, 100993) c6( ) c7(UR, QK, xw, cQV, nA); #c1(TSEN34) c2(XP_005259298) c3(tRNA-splicing endonuclease subunit Sen34 isoform X1) c4(276056) c5(276057, 276058, 276059) c6( ) c7(UR, QK, xw, cQV, nA); #c1(TSEN54) c2(NP_997229) c3(tRNA-splicing endonuclease subunit Sen54) c4(265238, 265239) c5(265240, 265241, 265242, 265243, 265244, 265245, 265246) c6( ) c7(cQX, cyv, aJI, UR, cQW, nA, QK, xw); #c1(TSEN54) c2(XP_006721884) c3(tRNA-splicing endonuclease subunit Sen54 isoform X5) c4( ) c5(294361, 294362, 294363, 294364, 294365) c6( ) c7(cQX, cyv, aU, UR, cQW, nA, QK, xw); #c1(TSEN54) c2(XP_011522919) c3(tRNA-splicing endonuclease subunit Sen54 isoform X3) c4(317421) c5( ) c6( ) c7(cQX, cyv, aJI, UR, cQW, nA, QK, xw); #c1(TSEN54) c2(XP_011522920) c3(tRNA-splicing endonuclease subunit Sen54 isoform X4) c4( ) c5(317422, 317423, 317424, 317425, 317426, 317427) c6( ) c7(cQX, cyv, aJI, UR, cQW, nA, QK, xw); #c1(TSFM) c2(NP_001166166) c3(elongation factor Ts, mitochondrial isoform 3 precursor) c4(68379) c5(68380, 68381, 68382, 68383, 68384) c6( ) c7(IM, hb, cc, cQY); #c1(TSFM) c2(NP_001166167) c3(elongation factor Ts, mitochondrial isoform 1 precursor) c4(68385) c5(68386, 68387, 68388, 68389, 68390, 68391) c6( ) c7(IM, hb, cc, cQY); #c1(TSFM) c2(NP_001166168) c3(elongation factor Ts, mitochondrial isoform 4 precursor) c4(68392) c5(68393, 68394, 68395, 68396, 68397, 68398) c6( ) c7(IM, hb, cc, cQY); #c1(TSFM) c2(NP_005717) c3(elongation factor Ts, mitochondrial isoform 2 precursor) c4(158798, 158799) c5(158800, 158801, 158802, 158803, 158804, 158805, 158806, 158807, 158808, 158809, 158810, 158811, 158812) c6( ) c7(IM, hb, cc, cQY); #c1(TSG101) c2(NP_006283) c3(tumor susceptibility gene 101 protein) c4(163584, 163585) c5(163586, 163587, 163588, 163589, 163590, 163591) c6( ) c7(T, if, jx, aBz, jh, aA, ci, qN, ad, er, ft, hd, aT, h, dL, K, bU, pk, ae, Jl, aW, bZC, aq, lo, aF, iR, t, bz, ck, nB, gX, at, m, im, bt, B); #c1(TSG101) c2(XP_005253165) c3 (tumor susceptibility gene 101 protein isoform X1) c4( ) c5(272227, 272228, 272229, 272230, 272231, 272232) c6( ) c7(T, if, jx, aBz, jh, aA, ci, qN, ad, er, ft, hd, aT, h, dL, K, bU, pk, ae, Jl, aW, bZC, aq, lo, aF, iR, t, bz, ck, nB, gX, at, m, im, bt, B); #c1(TSGA10) c2(NP_878915) c3(testis-specific gene 10 protein) c4( ) c5(257173, 257174, 257175, 257176, 257177, 257178, 257179, 257180, 257181, 257182, 257183, 257184, 257185, 257186, 257187, 257188, 257189, 257190, 257191) c6( ) c7(ck, x, T, B, Y, hF, dL, k, hf, t, X, ad); #c1(TSGA10) c2(XP_011510236) c3(testis-specific gene 10 protein isoform X2) c4(300831, 300832, 300833) c5(300834) c6( ) c7(ck, x, T, B, Y, hF, dL, k, hf, t, X, ad); #c1(TSGA10) c2(XP_011510237) c3(testis-specific gene 10 protein isoform X3) c4( ) c5(300835, 300836, 300837, 300838) c6( ) c7(ck, x, T, B, Y, hF, dL, k, hf, t, X, ad); #c1(TSGA10) c2(XP_011510246) c3(testis-specific gene 10 protein isoform X4) c4( ) c5(300839, 300840, 300841, 300842, 300843, 300844) c6( ) c7(ck, x, T, B, Y, hF, dL, k, hf, t, X, ad); #c1(TSHR) c2(XP_005268094) c3 (thyrotropin receptor isoform X3) c4(281502, 281503) c5(281504, 281505, 281506, 281507, 281508, 281509, 281510, 281511, 281512, 281513) c6( ) c7(bDV, cJY, T, aC, Uc, AS, FR, sQ, wJ, sw, Dd, aw, cRa, bzb, cz, cRe, bWZ, hz, bBV, fd, ci, ml, cRg, biO, cRi, V, cRh, Vp, hd, xy, av, mP, cQZ, cRb, cRc, nv, akU, Db, cje, aT, hD, LA, Fx, lQ, AN, bDW, yF, aQy, li, bDT, ak, zQ, Ix, s, bwB, cRk, aus, mR, ajP, cRd, bxF, xv, Hu, f, Bu, cm, cRj, dr, qJ, pv, nM, sn, nB, cxd, Lj, bNJ, hr, uo, ut, od, gT, cRf, jr); #c1(TSHR) c2(XP_005268096) c3(thyrotropin receptor isoform X1) c4(281514) c5(281515, 281516, 281517, 281518, 281519, 281520) c6( ) c7(bDV, cJY, T, aC, Uc, AS, FR, sl, wJ, sw, Dd, aw, cRa, bzb, cz, cRe, bWZ, hz, bBV, fd, ci, mO, cRg, biO, cRi, V, cRh, Vp, hd, xy, av, mP, cQZ, cRb, cRc, nv, akU, Db, cje, aT, HD, LA, Fx, lQ, AN, bDW, yF, aQy, li, bDT, ak, zl, lx, s, bwB, cRk, aus, mR, ajP, cRd, bxF, xv, Hu, f, Bu, cm, cRj, dr, qJ, pv, nM, sn, nB, cxd, Lj, bNJ, hr, uo, ut, ol, gT, cRf, jr); #c1(TSHR) c2(XP_006720308) c3(thyrotropin receptor isoform X2) c4(293345) c5(293346, 293347, 293348, 293349, 293350, 293351) c6( ) c7(bDV, cJY, T, aC, Uc, AS, fR, sQ, wJ, sw, Dd, aw, cRa, bzb, cz, cRe, bWZ, hz, bBV, fd, ci, mO, cRg, biO, cRi, V, cRh, Vp, hd, xy, av, mP, cQZ, cRb, cRc, nv, akU, Db, cje, aT, hD, LA, Fx, lQ, AN, bDW, yF, aQy, li, bDT, ak, zQ, lx, s, bwB, cRk, aus, mR, ajP, cRd, bxF, xv, Hu, f, Bu, cm, cRj, dr, qJ, pv, nM, sn, nB, cxd, Lj, bNJ, hr, uo, ut, od, gT, cRf, jr); #c1(TSHR) c2(XP_011535421) c3(thyrotropin receptor isoform X4) c4( ) c5(332462, 332463, 332464, 332465, 332466, 332467) c6( ) c7(bDV, cJY, T, aC, Uc, AS, fR, sQ, wJ, sw, Dd, aw, cRa, bzb, cz, cRe, bWZ, hz, bBV, fd, ci, mO, cRg, biO, cRi, V, cRh, Vp, hd, xy, av, mP, cQZ, cRb, cRc, nv, akU, Db, cje, aT, HD, LA, Fx, lQ, AN, bDW, yF, aQy, li, bDT, ak, zQ, lx, s, bwB, cRk, aus, mR, ajP, cRd, bxF, xv, Hu, f, Bu, cm, cRj, dr, qJ, pv, nM, sn, nB, cxd, Lj, bNJ, hr, uo, ut, oQ, gT, cRf, jr); #c1(TSHZ1) c2(NP_005777) c3(teashirt homolog 1) c4(159249) c5(159250, 159251, 159252, 159253, 159254, 159255, 159256, 159257, 159258, 159259) c6( ) c7(T, AS, l, cz, rf, aR, ad, yn, dP, iu, akN, sO, aOo, aT, bK, AN, H, on, ae, af, eu, aIL, ak, d, bf, aW, sq, aq, Qm, bu, xw, qJ, L, tj, ac, ky, kB, c, qe, rR, Kv, eM, mx, kq, bel, ER, R, be, y, B, cRI); #c1(TSHZ2) c2(NP_001180350) c3(teashirt homolog 2 isoform 2) c4(73858) c5(73859, 73860, 73861, 73862, 73863, 73864) c6( ) c7(aw, fX, ad): #c1(TSHZ2) c2(NP_775756) c3(teashirt homolog 2 isoform 1) c4(250182) c5(250183, 250184, 250185, 250186, 250187, 250188) c6( ) c7(aw, fX, ad); #c1(TSHZ3) c2(NP_065907) c3(teashirt homolog 3) c4(206169) c5(206170, 206/71, 206/72, 206/73, 206174, 206175) c6( ) c7(fX, c, aW, cRn, al, cz, j, Ea, cRm, ae, ad); #c1(TSLP) c2(NP_149024) c3(thymic stromal lymphopoietin isoform 1 precursor) c4(226806, 226807) c5(226808, 226809, 226810, 226811, 226812, 226813, 226814) c6( ) c7(bM, T, dD, Dw, akc, ea, fG, aD, jx, cz, bBR, v, qj, eq, bRn, aVU, Jx, apt, lR, ai, aoH, dP, x, afm, dn, aT, cU, ji, K, qM, lM, cgQ, Us, sj, aa, aW, sp, az, hl, cr, akY, lo, iV, eE, pb, ck, cT, yl, DW, cj, at, k, yH, ei, i, hE, y, pC); #c1(TSLP) c2(XP_011542000) c3(thymic stromal lymphopoietin isoform X1) c4( ) c5(340493, 340494, 340495, 340496, 340497) c6( ) c7(bM, T, dD, Dw, akc, ea, fG, aD, jx, cz, bBR, v, qj, eq, bRn, aVU, Jx, apt, lR, ai, aoH, dP, x, afm, dn, aT, cU, ji, K, qM, IM, cgQ, Us, sj, aa, aW, sp, az, hl, cr, akY, lo, iV, eE, pb, ck, cT, yl, DW, cj, at, k, yH, ei, i, hE, y, pC); #c1(TSN) c2(NP_001248330) c3(translin isoform 2) c4(93694) c5(93695, 93696, 93697, 93698, 93699) c6( ) c7(T, ac, au, jh, Xu, Le, B, Hr); #c1(TSN) c2(NP_004613) c3(translin isoform 1) c4(148/72, 148173) c5(148174, 148175, 148176, 148177, 148178, 148179, 148180, 148181, 148182, 148183, 148184, 148185, 148186) c6( ) c7(T, ac, au, jh, Xu, Le, B, Hr); #c1(TSNAX) c2(NP_005990) c3(translin-associated protein X) c4(161050, 161051) c5(161052, 161053, 161054, 161055, 161056, 161057, 161058, 161059, 161060, 161061, 161062, 161063) c6( ) c7(T, bO, vL, aqm, bK, ahg, cl, Z, he, bN); #c1(TSPAN10) c2(NP_001277141) c3(tetraspanin-10 isoform a) c4(109119, 109/20) c5(109121) c6( ) c7(aw, ac); #c1(TSPAN10) c2(NP_114151) c3(tetraspanin-10 isoform b) c4( ) c5(222361, 222362, 222363, 222364, 222365, 222366) c6( ) c7(aw, ac); #c1(TSPAN10) c2(XP_011523627) c3(tetraspanin-10 isoform X1) c4( ) c5(318135, 318136, 318137, 318138, 318139, 318140) c6( ) c7(aw, ac); #c1(TSPAN11) c2(XP_011518982) c3(tetraspanin-11 isoform X1) c4(313440, 313441) c5(313442, 313443, 313444, 313445, 313446, 313447, 313448, 313449, 313450, 313451, 313452, 313453, 313454, 313455, 313456, 313457) c6( ) c7(eZ); #c1(TSPAN12) c2(XP_011514296) c3(tetraspanin-12 isoform X1) c4(306942, 306943, 306944, 306945, 306946) c5(306947, 306948, 306949, 306950, 306951, 306952, 306953, 306954, 306955, 306956, 306957, 306958, 306959, 306960, 306961, 306962, 306963, 306964, 306965, 306966, 306967, 306968, 306969, 306970, 306971, 306972, 306973, 306974, 306975, 306976, 306977, 306978, 306979, 306980) c6( ) c7(bN, T, cRo, byl, byj); #c1(TSPAN13) c2(NP_055214) c3(tetraspanin-13) c4(179876) c5(179877, 179878, 179879, 179880, 179881, 179882) c6( ) c7(aW, ae, dA, ad, aT); #c1(TSPAN14) c2(NP_001121781) c3(tetraspanin-14 isoform 2) c4(46144) c5(46145, 46146, 46147, 46148, 46149, 46150) c6( ) c7(bqW, eM); #c1(TSPAN14) c2(NP_112189) c3(tetraspanin-14 isoform 1) c4(220675, 220676, 220677, 220678) c5(220679, 220680, 220681, 220682, 220683, 220684, 220685, 220686, 220687, 220688, 220689, 220690, 220691, 220692, 220693, 220694, 220695, 220696, 220697, 220698, 220699, 220700, 220701, 220702, 220703, 220704, 220705, 220706, 220707, 220708, 220709, 220710, 220711, 220712, 220713, 220714, 220715, 220716) c6( ) c7(bqW, eM); #c1(TSPAN14) c2(XP_011538520) c3(tetraspanin-14 isoform X2) c4(336271) c5( ) c6( ) c7(bqW, eM); #c1(TSPAN16) c2(NP_001269438) c3(tetraspanin-16 isoform 2) c4( ) c5(101668) c6( ) c7(x, k); #c1(TSPAN16) c2(NP_036598) c3(tetraspanin-16 isoform 1) c4(174708, 174709) c5(174710, 174711, 174712, 174713, 174714, 174715) c6( ) c7(x, k); #c1(TSPAN16) c2(XP_011526203) c3(tetraspanin-16 isoform X1) c4( ) c5(320472, 320473, 320474, 320475, 320476) c6( ) c7(x, k); #c1(TSPAN18) c2(XP_006718435) c3(tetraspanin-18 isoform X2) c4(291861, 291862) c5(291863, 291864, 291865, 291866, 291867, 291868, 291869, 291870, 291871, 291872) c6( ) c7(pf); #c1(TSPAN18) c2(XP_011518761) c3(tetraspanin-18 isoform X1) c4(313153, 313154) c5(313155, 313156, 313157, 313158, 313159, 313160, 313161, 313162, 313163, 313164, 313165, 313166, 313167, 313168, 313169, 313170, 313/71, 313172) c6( ) c7(pf); #c1(TSPAN31) c2(NP_005972) c3(tetraspanin-31) c4(160891) c5(160892, 160893, 160894, 160895, 160896, 160897) c6( ) c7(T, sw, ha, aA, bS, ct, iJ, hW, yg, cU, h, d, Or, ff, eH, aF, en, eF, cm, Z, iK, QG, au, QC, AC, ciI, nz, bt); #c1(TSPAN32) c2(NP_620591) c3(tetraspanin-32) c4(237858) c5(237859, 237860, 237861, 237862, 237863) c6( ) c7(ds, jZ, aE); #c1(TSPAN32) c2(XP_011518113) c3(tetraspanin-32 isoform X3) c4( ) c5(312107) c6( ) c7(ds, jZ, aE); #c1(TSPAN32) c2(XP_011518124) c3(tetraspanin-32 isoform X14) c4(312108) c5(312109, 312110, 312111, 312112, 312113, 312114) c6( ) c7(ds, jZ, aE); #c1(TSPAN33) c2(NP_848657) c3(tetraspanin-33) c4(254089) c5(254090, 254091, 254092, 254093, 254094, 254095) c6( ) c7(fM, jh, eZ, bw, bt, eu); #c1(TSPAN4) c2(XP_005253159) c3(tetraspanin-4 isoform X1) c4(272178, 272179, 272180, 272181, 272182) c5(272183, 272184, 272185, 272186, 272187, 272188, 272189, 272190, 272191, 272192, 272193, 272194, 272195, 272196, 272197, 272198, 272199, 272200, 272201, 272202, 272203, 272204, 272205, 272206, 272207, 272208, 272209, 272210, 272211, 272212, 272213, 272214, 272215, 272216, 272217, 272218, 272219, 272220, 272221, 272222, 272223, 272224, 272225, 272226) c6( ) c7(T, dt); #c1(TSPAN4) c2(XP_011518638) c3(tetraspanin-4 isoform X3) c4(312964) c5(312965, 312966, 312967) c6( ) c7(T, dt); #c1(TSPAN4) c2(XP_011518643) c3(tetraspanin-4 isoform X2) c4( ) c5(312968, 312969, 312970, 312971, 312972, 312973, 312974, 312975, 312976, 312977, 312978, 312979, 312980) c6( ) c7(T, dt); #c1(TSPAN6) c2(NP_003261) c3(tetraspanin-6 isoform a) c4(136910, 136911) c5(136912, 136913, 136914, 136915, 136916, 136917, 136918, 136919, 136920, 136921, 136922, 136923) c6( ) c7(vr); #c1(TSPAN7) c2(NP_004606) c3(tetraspanin-7) c4(148104, 148105, 148106, 148107, 148108) c5(148109, 148110, 148111, 148112, 148113, 148114, 148115, 148116, 148117, 148118, 148119, 148120, 148121, 148122, 148123, 148124, 148125, 148126, 148127, 148128, 148129, 148130, 148131, 148132, 148133, 148134, 148135, 148136, 148137, 148138, 148139, 148140) c6(148141, 148142) c7(T, py, iW, jA, cRp, nA, ae, af, ad); #c1(TSPAN8) c2(XP_006719646) c3(tetraspanin-8 isoform X1) c4(292852) c5(292853, 292854, 292855, 292856, 292857, 292858, 292859, 292860, 292861, 292862, 292863, 292864, 292865, 292866) c6( ) c7(bL, T, e, ht, DV, al, aj, v, ci, bh, q, J, h, bK, Y, r, dS, gk, abV, W, cA, bE, uJ, y, B); #c1(TSPAN9) c2(NP_001161792) c3(tetraspanin-9) c4(66016) c5(66017, 66018, 66019, 66020, 66021, 66022, 66023, 66024, 66025, 66026, 66027, 66028) c6( ) c7(bL, awi, ac, af); #c1(TSPEAR) c2(NP_001258966) c3(thrombospondin-type laminin G domain and EAR repeat-containing protein isoform 2) c4( ) c5(96835, 96836, 96837, 96838, 96839, 96840) c6( ) c7(ha, cRq, cA); #c1(TSPEAR) c2(NP_659428) c3(thrombospondin-type laminin G domain and EAR repeat-containing protein isoform 1 precursor) c4(239813, 239814, 239815) c5(239816, 239817, 239818, 239819, 239820, 239821, 239822, 239823, 239824, 239825, 239826, 239827) c6( ) c7(ha, cRq, cA); #c1(TSP0) c2(NP_001243459) c3(translocator protein) c4(90852, 90853) c5(90854, 90855, 90856, 90857, 90858, 90859, 90860, 90861, 90862, 90863, 90864, 90865, 90866, 90867, 90868, 90869, 90870, 90871, 90872, 90873) c6( ) c7(bM, T, aCA, AS, dA, aw, bO, jh, oY, aj, bS, qc, ct, ci, bX, Fb, wV, V, ww, q, J, ad, aXI, eA, XX, hd, aT, hD, ahr, cy, dL, bK, K, h, rE, H, nJ, ae, agK, d, lR, tb, aW, Y, ff, r, en, as, Z, cm, xe, bz, iK, bN, hq, bax, L, YE, nB, dB, wO, U, tV, be, hh, Ro); #c1(TSP02) c2(XP_011512699) c3(translocator protein 2 isoform X1) c4(303728) c5(303729, 303730, 303731, 303732, 303733, 303734, 303735, 303736, 303737, 303738) c6( ) c7(at, iW); #c1(TSPY1) c2(NP_003299) c3(testis-specific Y-encoded protein 1 isoform TSPY-L) c4(137233) c5(137234, 137235, 137236, 137237, 137238, 137239, 137240, 137241, 137242, 137243, 137244, 137245) c6( ) c7(T, gU, wu, oY, UI, rc, ad, UE, BI, BJ, PI, aT, h, wm, ae, vT, aW, aq, bu, iR, pb, aeH, Lb, bxG, Le, B); #c1(TSPY10) c2(NP_001269398) c3(testis-specific Y-encoded protein 10) c4(101568) c5(101569, 101570, 101571, 101572, 101573, 101574, 101575, 101576, 101577, 101578, 101579, 101580, 101581, 101582, 101583) c6( ) c7(T, gU, wu, oY, UI, ad, BI, BJ, PI, aT, h, wm, ae, vT, aW, aq, bu, iR, pb, aeH, bxG, Le, B); #c1(TSPY10) c2(XP_002344239) c3(testis-specific Y-encoded protein 10 isoform X1) c4(266103) c5(266104, 266105, 266106, 266107, 266108, 266109, 266110, 266111, 266112, 266113, 266114) c6( ) c7(T, gU, wu, oY, UI, ad, BI, BJ, PI, aT, h, wm, ae, vT, aW, aq, bu, iR, pb, aeH, bxG, Le, B); #c1(TSPY3) c2(XP_011542306) c3(testis-specific Y-encoded protein 3 isoform X2) c4(340909) c5(340910, 340911) c6( ) c7(T, gU, wu, oY, UI, ad, BI, BJ, PI, aT, h, wm, ae, vT, aW, aq, bu, iR, pb, aeH, bxG, Le, B); #c1(TSPY4) c2(NP_001157943) c3(testis-specific Y-encoded protein 4) c4(62929, 62930, 62931, 62932) c5(62933, 62934, 62935, 62936, 62937, 62938, 62939, 62940, 62941, 62942, 62943, 62944) c6( ) c7(T, gU, wu, oY, UI, ad, BI, BJ, PI, aT, h, wm, ae, vT, aW, aq, bu, iR, pb, aeH, bxG, Le, B); #c1(TSPY4) c2(XP_005272800) c3(testis-specific Y-encoded protein 4 isoform X1) c4(284184, 284185, 284186) c5(284187, 284188, 284189, 284190, 284191, 284192, 284193, 284194, 284195, 284196, 284197, 284198, 284199, 284200, 284201, 284202, 284203) c6( ) c7(T, gU, wu, oY, UI, ad, BI, BJ, PI, aT, h, wm, ae, vT, aW, aq, bu, iR, pb, aeH, bxG, Le, B); #c1(TSPYL1) c2(NP_003300) c3(testis-specific Y-encoded-like protein 1) c4(137246, 137247) c5(137248, 137249, 137250, 137251, 137252, 137253, 137254, 137255, 137256, 137257, 137258, 137259) c6( ) c7(EZ, aeT, eA, gU, cRr); #c1(TSPYL2) c2(NP_071400) c3(testis-specific Y-encoded-like protein 2) c4(211429) c5(211430, 211431, 211432, 211433, 211434, 211435) c6( ) c7(NU, T, eL, eW, eM, alc, xR, adw, hL, jh, h, bz, B); #c1(TSPYL4) c2(NP_067680) c3 (testis-specific Y-encoded-like protein 4) c4(209290) c5(209291, 209292, 209293, 209294, 209295, 209296) c6( ) c7(Y); #c1(TSPYL5) c2(NP_277047) c3(testis-specific Y-encoded-like protein 5) c4(228807) c5(228808, 228809, 228810, 228811, 228812, 228813) c6( ) c7(aW, aA, aT, aF, eA, ad); #c1(TSR1) c2(NP_060598) c3(pre-rRNA-processing protein TSR1 homolog) c4(196761) c5(196762, 196763, 196764, 196765, 196766, 196767) c6( ) c7(e); #c1(TSSC1) c2(NP_003301) c3(protein TSSC1) c4(137260) c5(137261, 137262, 137263, 137264, 137265, (37266) c6( ) c7(IB); #c1(TSSC1) c2(XP_011508685) c3(protein TSSC1 isoform X1) c4( ) c5(299317) c6( ) c7(IB); #c1(TSSK1B) c2(NP_114417) c3(testis-specific serine/threonine-protein kinase 1) c4(222685) c5(222686, 222687, 222688, 222689, 222690, 222691) c6( ) c7(gU, IR, pb); #c1(TSSK2) c2(NP_443732) c3(testis-specific serine/threonine-protein kinase 2) c4(230066) c5(230067, 230068, 230069, 230070, 230071, 230072) c6( ) c7(uo, pb); #c1(TSSK4) c2(NP_001171668) c3(testis-specific serine/threonine-protein kinase 4 isoform 1) c4(71243) c5(71244, 71245, 71246, 71247, 71248, 71249) c6( ) c7(vH, gU, ND); #c1(TSSK4) c2(NP_777604) c3(testis-specific serine/threonine-protein kinase 4 isoform 2) c4(251318, 251319) c5(251320, 251321, 251322, 251323, 251324, 251325) c6( ) c7(vH, gU, ND); #c1(TST) c2(NP_003303) c3(thiosulfate sulfurtransferase) c4(137274, 137275, 137276) c5(137277, 137278, 137279, 137280, 137281, 137282, 137283, 137284, 137285, 137286, 137287, 137288, 137289, 137290, 137291, 137292, 137293, 137294, 137295, 137296, 137297, 137298) c6( ) c7(bGg, LX, nx, aeu, cap, ad, Sr, h, ji, aqX, db, xX, lR, nk, nC, W, nb, ig, cvP, uJ, nw, B, LT); #c1(TSTA3) c2(NP_003304) c3(GDP-L-fucose synthase) c4( ) c5(137299, 137300, 137301, 137302, 137303, 137304, 137305, 137306, 137307, 137308, 137309, 137310, 137311, 137312, 137313, 137314, 137315, 137316, 137317, 137318, 137319, 137320, 137321) c6( ) c7(bz); #c1(TSTD1) c2(NP_001106676) c3(thiosulfate sulfurtransferase/rhodanese-like domain-containing protein 1 isoform 3) c4(42581) c5(42582, 42583, 42584, 42585, 42586, 42587) c6( ) c7(qt, T, Db); #c1(TSTD1) c2(NP_001106677) c3(thiosulfate sulfurtransferase/rhodanese-like domain-containing protein 1 isoform 2) c4(42588) c5(42589, 42590, 42591, 42592, 42593, 42594) c6( ) c7(qt, T, Db); #c1(TSTD1) c2(NP_001106678) c3(thiosulfate sulfurtransferase/rhodanese-like domain-containing protein 1 isoform 1) c4(42595, 42596) c5(42597, 42598, 42599, 42600, 42601, 42602, 42603, 42604, 42605, 42606, 42607, 42608) c6( ) c7(qt, T, Db); #c1(TTBK1) c2(NP_115927) c3(tau-tubulin kinase 1) c4(224732, 224733) c5(224734, 224735, 224736, 224737, 224738, 224739, 224740, 224741, 224742, 224743, 224744, 224745, 224746, 224747) c6( ) c7(Hx, af); #c1(TTBK1) c2(XP_011513248) c3(tau-tubulin kinase 1 isoform X2) c4( ) c5(305501, 305502) c6( ) c7(Hx, af); #c1(TTBK2) c2(NP_775771) c3(tau-tubulin kinase 2) c4(250250, 250251, 250252) c5(250253, 250254, 250255, 250256, 250257, 250258) c6( ) c7(alM, iA, kB, Y, rf, fr, cRs, xw); #c1(TTBK2) c2(XP_006720466) c3(tau-tubulin kinase 2 isoform X4) c4( ) c5(293517, 293518, 293519, 293520, 293521, 293522) c6( ) c7(alM, iA, kB, Y, rf, fr, cRs, xw); #c1(TTBK2) c2(XP_011519571) c3(tau-tubulin kinase 2 isoform X6) c4( ) c5(314231, 314232, 314233, 314234, 314235, 314236) c6( ) c7(alM, iA, kB, Y, rf, fr, cRs, xw); #c1(TTC1) c2(NP_001269429) c3 (tetratricopeptide repeat protein 1) c4(101635) c5(101636, 101637, 101638, 101639, 101640, 101641, 101642, 101643, 101644, 101645) c6( ) c7(hq); #c1(TTC12) c2(NP_060338) c3(tetratricopeptide repeat protein (2) c4(195466) c5(195467, 195468, 195469, 195470, 195471, 195472) c6( ) c7(Y, XY, Gd, aj, j, k, lB, FT); #c1(TTC12) c2(XP_011541187) c3(tetratricopeptide repeat protein 12 isoform X4) c4(339787) c5( ) c6( ) c7(Y, XY, Gd, aj, j, k, lB, FT); #c1(TTC12) c2(XP_011541189) c3 (tetratricopeptide repeat protein 12 isoform X6) c4(339788) c5(339789, 339790, 339791, 339792, 339793, 339794) c6( ) c7(Y, XY, Gd, aj, j, k, lB, FT); #c1(TTC12) c2(XP_011541191) c3(tetratricopeptide repeat protein 12 isoform X7) c4( ) c5(339795, 339796, 339797, 339798, 339799, 339800, 339801, 339802, 339803, 339804, 339805, 339806, 339807, 339808, 339809, 339810, 339811, 339812) c6( ) c7(Y, XY, Gd, aj, j, k, lB, FT); #c1(TTC17) c2(NP_060729) c3(tetratricopeptide repeat protein (7) c4(197489) c5(197490, 197491, 197492, 197493, 197494, 197495, 197496, 197497) c6( ) c7(cm, bM); #c1(TTC19) c2(NP_060245) c3(tetratricopeptide repeat protein 19, mitochondrial isoform 1 precursor) c4(194971, 194972, 194973) c5(194974, 194975, 194976, 194977, 194978, 194979, 194980, 194981, 194982, 194983, 194984, 194985, 194986, 194987, 194988, 194989, 194990) c6( ) c7(cRt, bYg, hb, afc, U, aqb, aR, aqe); #c1(TTC19) c2(XP_011522252) c3 (tetratricopeptide repeat protein 19, mitochondrial isoform X1) c4( ) c5(316888, 316889) c6( ) c7(cRt, bYg, hb, afc, U, aqb, aR, aqe); #c1(TTCZ1B) c2(NP_079029) c3(tetratricopeptide repeat protein 218) c4(216659) c5(216660, 216661, 216662, 216663, 216664, 216665) c6( ) c7(cRu, cRv, dy, Tp, lB, af); #c1(TTCZ1B) c2(XP_006712824) c3(tetratricopeptide repeat protein 218 isoform X3) c4(286882, 286883) c5(286884) c6( ) c7(cRu, cRv, dy, Tp, lB, af); #c1(TTC21B) c2(XP_011510172) c3 (tetratricopeptide repeat protein 218 isoform X1) c4(300740) c5( ) c6( ) c7(cRu, cRv, dy, Tp, lB, af); #c1(TTCZ1B) c2(XP_011510173) c3(tetratricopeptide repeat protein 21B isoform X2) c4( ) c5(300741, 300742, 300743, 300744, 300745, 300746) c6( ) c7(cRu, cRv, dy, Tp, lB, af); #c1(TTC28) c2(NP_001138890) c3(tetratricopeptide repeat protein 28) c4(58101) c5(58102, 58103, 58104, 58105, 58106, 58107) c6( ) c7(v, q, r); #c1(TTC29) c2(NP_001287690) c3(tetratricopeptide repeat protein 29 isoform 1) c4(112544, 112545, 112546) c5(112547, 112548, 112549, 112550, 112551, 112552, 112553, 112554, 112555, 112556, 112557, 112558, 112559, 112560, 112561, 112562, 112563, 112564, 112565, 112566, 112567, 112568) c6( ) c7(sj); #c1(TTC29) c2(XP_006714398) c3(tetratricopeptide repeat protein 29 isoform X1) c4(287827) c5(287828) c6( ) c7(j); #c1(TTC29) c2(XP_006714399) c3 (tetratricopeptide repeat protein 29 isoform X3) c4(287829) c5( ) c6( ) c7(j); #c1(TTC29) c2(XP_011530612) c3(tetratricopeptide repeat protein 29 isoform X6) c4( ) c5(326721, 326722, 326723, 326724, 326725, 326726) c6( ) c7(j); #c1(TTC3) c2(NP_001001894) c3(E3 ubiquitin-protein ligase TTC3) c4(19113, 19114) c5(19115, 19116, 19117, 19118, 19119, 19120, 19121, 19122, 19123, 19124, 19125, 19126, 19127, 19128, 19129, 19130, 19131, 19132, 19133, 19134) c6( ) c7(f, anj, A); #c1(TTC3) c2(XP_005261109) c3(E3 ubiquitin-protein ligase TTC3 isoform X3) c4(277212) c5(277213, 277214, 277215, 277216, 277217, 277218) c6( ) c7(f, anj, A); #c1(TTC3) c2(XP_005261113) c3(E3 ubiquitin-protein ligase TTC3 isoform X6) c4(277219) c5( ) c6( ) c7(f, anj, A); #c1(TTC3) c2(XP_011528044) c3(E3 ubiquitin-protein ligase TTC3 isoform X8) c4( ) c5(323139, 323140, 323141, 323142, 323143, 323144, 323145) c6( ) c7(f, anj, A); #c1(TTC37) c2(NP_055454) c3(tetratricopeptide repeat protein 37) c4(181251, 181252) c5(181253, 181254, 181255, 181256, 181257, 181258, 181259, 181260, 181261, 181262, 181263, 181264) c6( ) c7(aYD); #c1(TTC39A) c2(NP_001284592) c3 (tetratricopeptide repeat protein 39A isoform 4) c4(112100) c5(112101, 112102, 112103, 112104, 112105, 112106) c6( ) c7(aW, aj, aq); #c1(TTC39A) c2(NP_001284594) c3(tetratricopeptide repeat protein 39A isoform 6) c4(112107) c5(112108, 112109, 112110, 112111, 112112, 112113) c6( ) c7(aW, aj, aq); #c1(TTC39A) c2(NP_001284595) c3 (tetratricopeptide repeat protein 39A isoform 7) c4( ) c5(112114, 112115, 112116, 112117, 112118, 112119, 112120, 112121, 112122, 112123) c6( ) c7(aW, aj, aq); #c1(TTC39A) c2(XP_011539356) c3(tetratricopeptide repeat protein 39A isoform X1) c4( ) c5(337438, 337439) c6( ) c7(aW, aj, aq); #c1(TTC39B) c2(NP_001161812) c3(tetratricopeptide repeat protein 39B isoform 3) c4(66061, 66062) c5(66063, 66064, 66065, 66066, 66067, 66068) c6( ) c7(bL, nX, j, aj); #c1(TTC39B) c2(NP_001161813) c3(tetratricopeptide repeat protein 398 isoform 4) c4(66069) c5(66070, 66071, 66072, 66073, 66074, 66075) c6( ) c7(bL, nX, j, aj); #c1(TTC39B) c2(NP_689787) c3 (tetratricopeptide repeat protein 39B isoform 1) c4(243961, 243962) c5(243963, 243964, 243965, 243966, 243967, 243968, 243969, 243970, 243971, 243972, 243973, 243974, 243975) c6( ) c7(bL, nX, j, aj); #c1(TTC39B) c2(XP_011516035) c3(tetratricopeptide repeat protein 39B isoform X2) c4( ) c5(309238, 309239, 309240, 309241, 309242) c6( ) c7(bL, nX, j, aj); #c1(TTC5) c2(NP_612385) c3(tetratricopeptide repeat protein 5) c4(236328) c5(236329, 236330, 236331, 236332, 236333, 236334) c6( ) c7(ck, T, ci, K, bt, iu, dx); #c1(TTC6) c2(XP_011535733) c3(tetratricopeptide repeat protein 6 isoform X3) c4( ) c5(332969, 332970, 332971, 332972, 332973, 332974, 332975, 332976, 332977, 332978) c6( ) c7(cRw, dL, cd, pN); #c1(TTC7A) c2(NP_001275882) c3(tetratricopeptide repeat protein 7A isoform 3) c4( ) c5(107270, 107271, 107272, 107273, 107274, 107275, 107276, 107277, 107278, 107279, 107280, 107281) c6( ) c7(fe, y, fq, bsz); #c1(TTC7A) c2(NP_001275884) c3(tetratricopeptide repeat protein 7A isoform 4) c4(107282) c5( ) c6( ) c7(fe, y, fq, bsz); #c1(TTC7A) c2(NP_065191) c3(tetratricopeptide repeat protein 7A isoform 2) c4(204515, 204516, 204517) c5(204518, 204519, 204520, 204521, 204522, 204523) c6( ) c7(fe, y, fq, bsz); #c1(TTC7A) c2(XP_011531301) c3(tetratricopeptide repeat protein 7A isoform X2) c4( ) c5(327300, 327301, 327302, 327303, 327304, 327305) c6( ) c7(fe, y, fq, bsz); #c1(TTC7B) c2(NP_001010854) c3(tetratricopeptide repeat protein 78) c4(23838, 23839, 23840) c5(23841, 23842, 23843, 23844, 23845, 23846, 23847, 23848, 23849, 23850, 23851, 23852, 23853) c6( ) c7(e, ac, M); #c1(TTC7B) c2(XP_011534800) c3(tetratricopeptide repeat protein 78 isoform X2) c4(331288) c5(331289, 331290, 331291, 331292, 331293, 331294, 331295, 331296, 331297, 331298, 331299) c6( ) c7(e, ac, M); #c1(TTC8) c2(NP_001275710) c3(tetratricopeptide repeat protein 8 isoform D) c4(107152) c5(107153, 107154, 107155, 107156, 107157, 107158) c6( ) c7(IR, cRx, cRy, v, Tp, nC, cd); #c1(TTC8) c2(NP_001275711) c3 (tetratricopeptide repeat protein 8 isoform E) c4(107/59) c5(107160, 107161, 107162, 107163, 107164, 107165) c6( ) c7(IR, cRx, cRy, v, Tp, nC, cd); #c1(TTC8) c2(NP_001275712) c3 (tetratricopeptide repeat protein 8 isoform F) c4( ) c5(107166, 107167, 107168, 107169, 107170, 107171, 107172, 107173, 107174, 107/75, 107/76) c6( ) c7(IR, cRx, cRy, v, Tp, nC, cd); #c1(TTC8) c2(NP_653197) c3(tetratricopeptide repeat protein 8 isoform A) c4(239018, 239019, 239020) c5(239021, 239022, 239023, 239024, 239025, 239026, 239027, 239028, 239029, 239030, 239031) c6( ) c7(IR, cRx, cRy, v, Tp, nC, cd); #c1(TTC8) c2(NP_938051) c3(tetratricopeptide repeat protein 8 isoform B) c4(259562, 259563) c5(259564, 259565, 259566, 259567, 259568, 259569) c6( ) c7(IR, cRx, cRy, v, Tp, nC, cd); #c1(TTC8) c2(NP_938052) c3(tetratricopeptide repeat protein 8 isoform C) c4(259570) c5(259571, 259572, 259573, 259574, 259575, 259576, 259577) c6( ) c7(IR, cRx, cRy, v, Tp, nC, cd); #c1(TTC8) c2(XP_011534736) c3(tetratricopeptide repeat protein 8 isoform X6) c4( ) c5(331200) c6( ) c7(IR, cRx, cRy, v, Tp, nC, cd); #c1(TTC9) c2(NP_056166) c3(tetratricopeptide repeat protein 9A) c4(186060) c5(186061, 186062, 186063, 186064, 186065, 186066) c6( ) c7(VR); #c1(TTC9B) c2(NP_689692) c3(tetratricopeptide repeat protein 9B) c4(243581) c5(243582, 243583, 243584, 243585, 243586, 243587) c6( ) c7(aqN); #c1(TTC9C) c2(NP_776171) c3 (tetratricopeptide repeat protein 9C) c4( ) c5(250919, 250920, 250921, 250922, 250923, 250924, 250925, 250926, 250927, 250928, 250929, 250930, 250931) c6( ) c7(ad); #c1(TTC9C) c2(XP_011543265) c3(tetratricopeptide repeat protein 9C isoform X2) c4(342059, 342060, 342061) c5(342062, 342063, 342064, 342065, 342066, 342067, 342068, 342069, 342070) c6( ) c7(ad); #c1(TTF1) c2(XP_006717336) c3(transcription termination factor 1 isoform X1) c4(290939, 290940) c5(290941, 290942, 290943, 290944, 290945, 290946, 290947, 290948, 290949, 290950, 290951, 290952) c6( ) c7(Fv, T, apr, ci, av, ft, hd, Db, RN, h, bpf, Fx, biQ, NE, AQ, ff, kt, ahl, ckd, en, g, bw, bz, Dy, nM, eM, nB, fD, gT, iu); #c1(TTF2) c2(XP_011540606) c3(transcription termination factor 2 isoform X4) c4(339101) c5(339102, 339103, 339104, 339105, 339106, 339107) c6( ) c7(Na, nM, aE, bwB, aMW, Nc, cBf, gT, aQy); #c1(TTI1) c2(NP_001290386) c3(TELO2-interacting protein 1 homolog) c4(115325) c5(115326, 115327, 115328, 115329, 115330, 115331, 115332, 115333, 115334, 115335, 115336, 115337, 115338, 115339) c6( ) c7(fd); #c1(TTI2) c2(NP_079391) c3(TELO2-interacting protein 2) c4(217905, 217906, 217907) c5(217908, 217909, 217910, 217911, 217912, 217913, 217914, 217915, 217916, 217917, 217918, 217919, 217920, 217921, 217922, 217923) c6( ) c7(cRz, nA); #c1(TTI2) c2(XP_011542967) c3(TELO2-interacting protein 2 isoform X1) c4( ) c5(341669, 341670, 341671, 341672, 341673, 341674) c6( ) c7(cRz, nA); #c1(TTK) c2(XP_011534401) c3(dual specificity protein kinase TTK isoform X1) c4(330852, 330853, 330854, 330855) c5(330856, 330857, 330858, 330859, 330860, 330861) c6( ) c7(T, os, aaH, ht, jh, aA, oZ, aJI, iu, lE, aE, hb, aT, bNj, dL, ey, hu, eL, af, kl, d, aW, Y, r, dq, kp, aF, iK, kP, c, kg, Lq, ati, wS); #c1(TTK) c2(XP_011534402) c3(dual specificity protein kinase TTK isoform X2) c4(330862) c5(330863, 330864, 330865, 330866, 330867, 330868, 330869, 330870, 330871, 330872, 330873, 330874, 330875, 330876, 330877, 330878, 330879, 330880, 330881, 330882, 330883, 330884, 330885, 330886) c6( ) c7(T, os, aaH, ht, jh, aA, oZ, aJI, iu, lE, aE, hb, aT, bNj, dL, ey, hu, eL, af, kl, d, aW, Y, r, dq, kp, aF, iK, kP, c, kg, Lq, ati, wS); #c1(TTL) c2(NP_714923) c3(tubulin-tyrosine ligase) c4(246625) c5(246626, 246627, 246628, 246629, 246630, 246631) c6( ) c7(cm, ae, ad); #c1(TTLL1) c2(NP_036395) c3 (probable tubulin polyglutamylase TTLL1) c4(173566, 173567, 173568) c5(173569, 173570, 173571, 173572, 173573, 173574, 173575, 173576, 173577, 173578, 173579, 173580, 173581, 173582, 173583, 173584, 173585, 173586) c6( ) c7(cm, ae, ad); #c1(TTLL10) c2(XP_011539472) c3(inactive polyglycylase TTLL10 isoform X4) c4( ) c5(337677) c6( ) c7(nx); #c1(TTLL11) c2(NP_001132914) c3(tubulin polyglutamylase TTLL11 isoform a) c4(52639) c5(52640, 52641, 52642, 52643, 52644, 52645) c6( ) c7(Na, Nc); #c1(TTLL11) c2(NP_919228) c3(tubulin polyglutamylase TTLL11 isoform b) c4(258275) c5(258276, 258277, 258278, 258279, 258280, 258281) c6( ) c7(Na, Nc); #c1(TTLL11) c2(XP_005251785) c3(tubulin polyglutamylase TTLL11 isoform X3) c4(271182) c5(271183, 271184, 271185, 271186, 271187, 271188, 271189) c6( ) c7(Na, Nc); #c1(TTLL12) c2(NP_055955) c3(tubulin-tyrosine ligase-like protein (2) c4(184672, 184673) c5(184674, 184675, 184676, 184677, 184678, 184679, 184680, 184681, 184682, 184683, 184684, 184685) c6( ) c7(bX); #c1(TTLL12) c2(XP_011528317) c3(tubulin-tyrosine ligase-like protein 12 isoform X1) c4( ) c5(323381) c6( ) c7(bX); #c1(TTLL3) c2(NP_001021100) c3(tubulin monoglycylase TTLL3) c4(29127, 29128, 29129) c5(29130, 29131, 29132, 29133, 29134, 29135, 29136, 29137, 29138, 29139, 29140, 29141, 29142, 29143, 29144, 29145, 29146, 29147, 29148, 29149) c6( ) c7(q); #c1(TTLL4) c2(NP_055455) c3(tubulin polyglutamylase TTLL4) c4(181265, 181266, 181267, 181268) c5(181269, 181270, 181271, 181272, 181273, 181274, 181275, 181276, 181277, 181278) c6( ) c7(kp, aE); #c1(TTLL4) c2(XP_006712939) c3(tubulin polyglutamylase TTLL4 isoform X3) c4( ) c5(286910, 286911, 286912, 286913, 286914, 286915, 286916, 286917, 286918, 286919, 286920, 286921, 286922, 286923, 286924, 286925, 286926, 286927) c6( ) c7(kp, aE); #c1(TTLL5) c2(NP_055887) c3(tubulin polyglutamylase TTLL5) c4(184175, 184176, 184177, 184/78) c5(184179, 184180, 184181, 184182, 184183, 184184, 184185, 184186, 184187, 184188, 184189, 184190, 184191, 184192, 184193, 184194, 184195, 184196, 184197, 184198, 184199, 184200, 184201, 184202) c6( ) c7(t, T, nx, m); #c1(TTLL6) c2(NP_001124390) c3(tubulin polyglutamylase TTLL6 isoform 1) c4(48790, 48791, 48792, 48793, 48794, 48795) c5(48796, 48797, 48798, 48799, 48800, 48801, 48802, 48803, 48804, 48805, 48806, 48807, 48808, 48809, 48810, 48811, 48812, 48813, 48814, 48815, 48816, 48817, 48818, 48819, 48820, 48821, 48822, 48823, 48824, 48825, 48826, 48827, 48828, 48829, 48830, 48831) c6( ) c7(cm, aw); #c1(TTLL6) c2(NP_775894) c3(tubulin polyglutamylase TTLL6 isoform 2) c4(250590, 250591) c5(250592, 250593, 250594, 250595, 250596, 250597, 250598, 250599, 250600, 250601, 250602, 250603) c6( ) c7(cm, aw); #c1(TTLL7) c2(XP_011540457) c3(tubulin polyglutamylase TTLL7 isoform X2) c4(338924) c5(338925, 338926, 338927, 338928, 338929, 338930) c6( ) c7(af); #c1(TTLL7) c2(XP_011540459) c3(tubulin polyglutamylase TTLL7 isoform X4) c4(338931) c5( ) c6( ) c7(af); #c1(TTLL8) c2(XP_011529043) c3(protein monoglycylase TTLL8 isoform X1) c4(324384, 324385) c5( ) c6( ) c7(ac); #c1(TTLL8) c2(XP_011529044) c3(protein monoglycylase TTLL8 isoform X2) c4(324386) c5(324387, 324388, 324389, 324390, 324391, 324392, 324393, 324394, 324395, 324396, 324397, 324398, 324399, 324400, 324401, 324402, 324403, 324404) c6( ) c7(ac); #c1(TTLL9) c2(NP_001008409) c3(probable tubulin polyglutamylase TTLL9) c4(23179) c5(23180, 23181, 23182, 23183, 23184, 23185, 23186, 23187, 23188, 23189, 23190) c6( ) c7(aq, kl); #c1(TTLL9) c2(XP_005260366) c3(probable tubulin polyglutamylase TTLL9 isoform X19) c4(276525) c5(276526, 276527, 276528, 276529, 276530) c6( ) c7(aq, kl); #c1(TTLL9) c2(XP_006723786) c3(probable tubulin polyglutamylase TTLL9 isoform X12) c4(295387) c5(295388, 295389, 295390, 295391, 295392) c6( ) c7(aq, kl); #c1(TTLL9) c2(XP_006723787) c3(probable tubulin polyglutamylase TTLL9 isoform X13) c4( ) c5(295393, 295394, 295395, 295396, 295397) c6( ) c7(aq, kl); #c1(TTLL9) c2(XP_011526933) c3(probable tubulin polyglutamylase TTLL9 isoform X7) c4( ) c5(321221) c6( ) c7(aq, kl); #c1(TTLL9) c2(XP_011526936) c3(probable tubulin polyglutamylase TTLL9 isoform X10) c4(321222) c5(321223, 321224, 321225, 321226, 321227, 321228) c6( ) c7(aq, kl); #c1(TTLL9) c2(XP_011526937) c3(probable tubulin polyglutamylase TTLL9 isoform X11) c4(321229) c5(321230) c6( ) c7(aq, kl); #c1(TTLL9) c2(XP_011526942) c3(probable tubulin polyglutamylase TTLL9 isoform X16) c4( ) c5(321231) c6( ) c7(aq, kl); #c1(TTLL9) c2(XP_011526948) c3(probable tubulin polyglutamylase TTLL9 isoform X24) c4(321232) c5(321233, 321234, 321235, 321236, 321237) c6( ) c7(aq, kl); #c1(TTN) c2(NP_001243779) c3(titin isoform N2BA) c4(91718) c5( ) c6( ) c7(gx, T, cRE, UU, aj, cRA, gf, ci, j, cRB, pd, cd, J, cRD, ad, cc, Ag, Ws, oj, Ak, kf, cy, vc, h, K, atO, rz, bDW, ae, CRC, li, hM, anR, xz, xs, Ad, wl, bwc, uY, dg, aYo, cRF, f, Kc, Bv, abV, wK, azK, km, mx, gZ, jr); #c1(TTN) c2(NP_001254479) c3(titin isoform IC) c4(94402, 94403) c5(94404, 94405, 94406, 94407, 94408, 94409, 94410, 94411, 94412, 94413, 94414, 94415, 94416, 94417, 94418) c6(94419, 94420) c7(gx, T, cRE, UU, aj, cRA, gf, ci, j, cRB, pd, cd, J, cRD, ad, cc, Ag, Ws, oj, Ak, kf, cy, vc, h, K, atO, rz, bDW, ae, cRC, li, hM, anR, xz, xs, Ad, wl, bwc, uY, dg, aYo, CRF, f, Kc, Bv, abV, wK, azK, km, mx, gZ, jr); #c1(TTN) c2(NP_003310) c3(titin isoform N2-B) c4( ) c5(137351, 137352, 137353, 137354, 137355, 137356) c6( ) c7(gx, T, CRE, UU, aj, cRA, gf, ci, jj, cRB, pd, cd, J, cRD, ad, cc, Ag, Ws, oj, Ak, kf, cy, vc, h, K, atO, rz, bDW, ae, cRC, li, hM, anR, xz, xs, Ad, wl, bwc, uY, dg, aYo, cRF, f, Kc, Bv, abV, wK, azK, km, mx, gZ, jr); #c1(TTN) c2(NP_596869) c3(titin isoform N2-A) c4(234947) c5(234948, 234949, 234950, 234951, 234952, 234953, 234954, 234955, 234956, 234957, 234958, 234959, 234960) c6( ) c7(gx, T, cRE, UU, aj, cRA, gf, ci, j, cRB, pd, cd, J, cRD, ad, cc, Ag, Ws, oj, Ak, kf, cy, vc, h, K, atO, rz, bDW, ae, cRC, li, hM, anR, xz, xs, Ad, wl, bwc, uY, dg, aYo, CRF, f, Kc, Bv, abV, wK, azK, km, mx, gZ, jr); #c1(TTN) c2(NP_596870) c3(titin isoform novex-3) c4(234961, 234962) c5(234963, 234964, 234965, 234966, 234967, 234968, 234969, 234970, 234971, 234972, 234973, 234974) c6( ) c7(gx, T, cRE, UU, aj, cRA, gf, ci, j, cRB, pd, cd, J, cRD, ad, cc, Ag, Ws, oj, Ak, kf, cy, vc, h, K, atO, rz, bDW, ae, cRC, li, hM, anR, xz, xs, Ad, wl, bwc, uY, dg, aYo, cRF, f, Kc, Bv, abV, wK, azK, km, mx, gZ, jr); #c1(TTN) c2(NP_597676) c3(titin isoform novex-1) c4( ) c5(234975) c6( ) c7(gx, T, cRE, UU, aj, cRA, gf, ci, j, cRB, pd, cd, J, cRD, ad, cc, Ag, Ws, oj, Ak, kf, cy, vc, h, K, atO, rz, bDW, ae, cRC, li, hM, anR, xz, xs, Ad, wl, bwc, uY, dg, aYo, cRF, f, Kc, Bv, abV, wK, azK, km, mx, gZ, jr); #c1(TTN) c2(XP_011510031) c3(titin isoform X1) c4(300661) c5(300662, 300663, 300664, 300665, 300666) c6(300667) c7(gx, T, cRE, UU, aj, cRA, gf, ci, j, cRB, pd, cd, J, cRD, ad, cc, Ag, Ws, oj, Ak, kf, cy, vc, h, K, atO, rz, bDW, ae, cRC, li, hM, anR, xz, xs, Ad, wl, bwc, uY, dg, aYo, cRF, f, Kc, Bv, abV, wK, azK, km, mx, gZ, jr); #c1(TTN) c2(XP_011510034) c3(titin isoform X4) c4(300668) c5(300669, 300670, 300671, 300672, 300673, 300674, 300675) c6( ) c7(gx, T, cRE, UU, aj, cRA, gf, ci, j, cRB, pd, cd, J, cRD, ad, cc, Ag, Ws, oj, Ak, kf, cy, vc, h, K, atO, rz, bDW, ae, cRC, li, hM, anR, xz, xs, Ad, wl, bwc, uY, dg, aYo, cRF, f, Kc, Bv, abV, wK, azk, km, mx, gZ, jr); #c1(TTPA) c2(NP_000361) c3(alpha-tocopherol transfer protein) c4(7853) c5(7854, 7855, 7856, 7857, 7858, 7859) c6( ) c7(cw, dD, bBr, aw, eq, bSj, aR, ad, PW, Lv, aU, h, bl, DG, ae, af, sb, lR, aXy, kH, rU, aq, EH, nC, KM, dr, rY, wT, aUn, KB, rW, U, cj, y, B); #c1(TTR) c2(NP_000362) c3(transthyretin precursor) c4(7860, 7861, 7862, 7863) c5(7864, 7865, 7866, 7867, 7868, 7869, 7870, 7871, 7872, 7873, 7874, 7875, 7876, 7877, 7878, 7879, 7880, 7881, 7882, 7883, 7884, 7885, 7886, 7887, 7888, 7889, 7890, 7891, 7892, 7893, 7894, 7895, 7896, 7897, 7898, 7899, 7900, 7901, 7902, 7903, 7904, 7905, 7906, 7907, 7908, 7909, 7910, 7911, 7912, 7913, 7914, 7915, 7916, 7917, 7918, 7919, 7920, 7921, 7922, 7923, 7924, 7925, 7926, 7927, 7928, 7929, 7930, 7931, 7932, 7933, 7934, 7935, 7936, 7937, 7938, 7939, 7940, 7941, 7942, 7943, 7944, 7945, 7946, 7947, 7948, 7949, 7950, 7951, 7952, 7953, 7954, 7955, 7956, 7957, 7958, 7959, 7960, 7961, 7962, 7963, 7964, 7965, 7966, 7967, 7968, 7969, 7970, 7971, 7972, 7973, 7974, 7975, 7976, 7977, 7978, 7979, 7980, 7981, 7982, 7983, 7984, 7985, 7986, 7987, 7988, 7989, 7990, 7991, 7992, 7993, 7994, 7995, 7996, 7997, 7998, 7999, 8000, 8001, 8002, 8003, 8004, 8005, 8006, 8007, 8008, 8009, 8010, 8011, 8012, 8013, 8014, 8015, 8016, 8017, 8018, 8019, 8020, 8021, 8022, 8023) c6( ) c7(T, bkx, aXW, cRP, ea, aKu, l, aKs, ci, aR, ip, Na, bnt, K, H, cRH, mH, aW, Zc, Ls, RK, dg, cm, cad, Kv, U, abX, R, ckG, dD, bzb, n, cQ, fr, ct, nA, cd, RD, alZ, ke, jz, cc, cty, KN, agW, cRR, bV, cRN, Df, mJ, aGL, A, cRM, ahJ, aq, kp, aay, dw, akv, aYo, Rn, iO, cRQ, abt, aiS, bk, anx, B, cRS, L, bL, DD, amp, asB, rf, cz, bX, eA, eJ, cRL, cRI, lg, aT, af, eu, jf, aaO, cRJ, f, zJ, tj, c, cRO, akp, aor, dy, bKY, gT, bt, bLx, cyG, ob, aaI, aKw, RH, OR, bS, eq, fu, iC, av, aop, Db, sO, ds, Dq, cRG, oW, vc, aNp, ji, vl, Yi, cRK, EH, Z, YZ, dr, aeb, bF, cDd, gZ, aS, Lz, hE, xg); #c1(TTYH1) c2(NP_001005367) c3(protein tweety homolog 1 isoform 2) c4(21507) c5(21508, 21509, 21510, 21511, 21512, 21513) c6( ) c7(dB); #c1(TTYH1) c2(NP_001188390) c3(protein tweety homolog 1 isoform 3) c4(79/11) c5(79112, 79113, 79114, 79115, 79116, 79117, 79118, 79119) c6( ) c7(dB); #c1(TTYH1) c2(NP_065710) c3(protein tweety homolog 1 isoform 1) c4(205019, 205020) c5(205021, 205022, 205023, 205024, 205025, 205026) c6( ) c7(dB); #c1(TTYH1) c2(XP_006726337) c3(protein tweety homolog 1 isoform X1) c4( ) c5(297142, 297143, 297144, 297145, 297146) c6( ) c7(dB); #c1(TTYH2) c2(NP_116035) c3(protein tweety homolog 2 isoform 1) c4(225266) c5(225267, 225268, 225269, 225270, 225271, 225272) c6( ) c7(fr, aaw, X); #c1(TTYH2) c2(NP_443101) c3(protein tweety homolog 2 isoform 2) c4( ) c5(229428, 229429, 229430, 229431, 229432) c6( ) c7(fr, aaw, X); #c1(TTYH2) c2(XP_005257881) c3(protein tweety homolog 2 isoform X1) c4(275397) c5(275398) c6( ) c7(fr, aaw, X); #c1(TUB) c2(NP_003311) c3(tubby protein homolog isoform a) c4(137357) c5(137358, 137359, 137360, 137361, 137362, 137363, 137364, 137365, 137366) c6(137367, 137368) c7(bL, alF, lR, nx, nw, v, ei, nk, nC, ad); #c1(TUB) c2(NP_813977) c3 (tubby protein homolog isoform b) c4(253022) c5(253023, 253024, 253025, 253026, 253027, 253028, 253029, 253030) c6(253031, 253032) c7(bL, alF, lR, nx, nw, v, ei, nk, nC, ad); #c1(TUBA1A) c2(NP_001257328) c3(tubulin alpha-IA chain isoform 1) c4(95/70) c5(95171, 95/72, 95173, 95174, 95175, 95/76, 95177, 95178, 95/79, 95180) c6( ) c7(Mo, afe, lq, FB, FD, bdU, cja, cRT, j, nA, bQ, QK, FI, ahZ); #c1(TUBA1A) c2(NP_001257329) c3(tubulin alpha-(A chain isoform 2) c4( ) c5(95181, 95182, 95183, 95184, 95185, 95186) c6( ) c7(Mo, afe, lq, FB, FD, bdU, cja, cRT, j, nA, bQ, QK, FI, ahZ); #c1(TUBA1B) c2(NP_006073) c3(tubulin alpha-18 chain) c4(161666) c5(161667, 161668, 161669, 161670, 161671, 161672) c6( ) c7(ble, if, bB, qN, ad, aqJ, bPG, aT, ae, bA, aW, Y, ff, FD, eZ, en, cl, bz, eM, bE, bdU, B); #c1(TUBA1C) c2(NP_001290046) c3(tubulin alpha-IC chain isoform b) c4(114960, 114961) c5(114962, 114963, 114964, 114965, 114966, 114967) c6( ) c7(ad, aA); #c1(TUBA1C) c2(NP_116093) c3(tubulin alpha-IC chain isoform c) c4(225347) c5(225348, 225349, 225350, 225351, 225352, 225353) c6( ) c7(ad, aA); #c1(TUBA3D) c2(NP_525125) c3(tubulin alpha-3C/D chain) c4(231181, 231182) c5(231183, 231184, 231185, 231186, 231187, 231188, 231189, 231190, 231191, 231192, 231193, 231194, 231195, 231196, 231197, 231198, 231199, 231200, 231201, 231202) c6( ) c7(bUh, aDZ, eM); #c1(TUBA4A) c2(NP_001265481) c3(tubulin alpha-4A chain isoform 2) c4( ) c5(98674, 98675, 98676, 98677, 98678, 98679, 98680) c6(98681, 98682) c7(eM, c, aT, eo, PJ, V); #c1(TUBA4A) c2(NP_005991) c3(tubulin alpha-4A chain isoform 1) c4(161064, 161065) c5(161066, 161067, 161068, 161069, 161070, 161071, 161072, 161073, 161074) c6(161075, 161076) c7(eM, c, aT, eo, PJ, V); #c1(TUBA8) c2(NP_001180343) c3(tubulin alpha-8 chain isoform 2) c4(73838) c5(73839, 73840, 73841, 73842, 73843, 73844) c6( ) c7(iH, af, bE, ble, X, hl, Zn, oh, jy, bK, ci, C, oi, dF, B); #c1(TUBA8) c2(NP_061816) c3(tubulin alpha-8 chain isoform 1) c4(200469) c5(200470, 200471, 200472, 200473, 200474, 200475, 200476) c6( ) c7(iH, af, bE, ble, X, hl, Zn, oh, jy, bK, ci, C, oi, dF, B); #c1(TUBB) c2(NP_001280142) c3(tubulin beta chain isoform c) c4(111370) c5(111371, 111372, 111373, 111374, 111375, 111376) c6( ) c7(es, T, av, QK, cF, iA, aW, e, mx, eZ, aT, fr, m, DY, t); #c1(TUBB) c2(NP_001280145) c3(tubulin beta chain isoform e) c4(111377, 111378) c5(111379, 111380, 111381, 111382, 111383, 111384, 111385, 111386, 111387, 111388, 111389, 111390, 111391, 111392, 111393, 111394, 111395, 111396, 111397, 111398, 111399, 111400, 111401, 111402, 111403, 111404, 111405, 111406, 111407, 111408, 111409, 111410, 111411, 111412, 111413, 111414, 111415, 111416, 111417, 111418, 111419, 111420, 111421, 111422, 111423, 111424, 111425, 111426, 111427, 111428, 111429, 111430, 111431, 111432, 111433, 111434, 111435, 111436, 111437, 111438, 111439, 111440, 111441, 111442, 111443, 111444, 111445, 111446, 111447, 111448, 111449, 111450, 111451, 111452, 111453, 111454, 111455, 111456, 111457, 111458, 111459, 111460, 111461) c6( ) c7(es, T, av, QK, cF, iA, aW, e, mx, eZ, aT, fr, m, DY, t); #c1(TUBB) c2(NP_821133) c3(tubulin beta chain isoform b) c4(253232, 253233, 253234, 253235, 253236, 253237) c5(253238, 253239, 253240, 253241, 253242, 253243, 253244, 253245, 253246, 253247, 253248, 253249, 253250, 253251, 253252, 253253, 253254, 253255, 253256, 253257, 253258, 253259, 253260, 253261, 253262, 253263, 253264, 253265, 253266, 253267, 253268, 253269, 253270, 253271, 253272, 253273, 253274, 253275, 253276, 253277, 253278, 253279, 253280, 253281, 253282, 253283, 253284, 253285, 253286, 253287, 253288, 253289, 253290, 253291, 253292, 253293, 253294, 253295, 253296, 253297, 253298, 253299, 253300, 253301, 253302, 253303, 253304, 253305, 253306, 253307, 253308, 253309, 253310, 253311, 253312, 253313, 253314, 253315, 253316, 253317, 253318, 253319, 253320, 253321, 253322, 253323, 253324, 253325, 253326, 253327, 253328, 253329, 253330, 253331, 253332, 253333, 253334, 253335, 253336, 253337, 253338, 253339, 253340, 253341, 253342, 253343, 253344, 253345, 253346, 253347, 253348, 253349, 253350, 253351, 253352) c6( ) c7(es, T, av, QK, cF, iA, aW, e, mx, eZ, aT, fr, m, DY, t); #c1(TUBB1) c2(NP_110400) c3(tubulin beta-1 chain) c4(220045) c5(220046, 220047, 220048, 220049, 220050, 220051) c6( ) c7(qq, e, aw, ac, cRU); #c1(TUBB2A) c2(NP_001060) c3(tubulin beta-2A chain) c4(34490) c5(34491, 34492, 34493, 34494, 34495, 34496) c6( ) c7(aA, bt, ha, cRV); #c1(TUBB2B) c2(NP_821080) c3(tubulin beta-28 chain) c4(253225) c5(253226, 253227, 253228, 253229, 253230, 253231) c6( ) c7(cRW, aJI, zQ, QK, lq, FB, FD, aT, ci, bxw, bQ, zP, cRX); #c1(TUBB3) c2(NP_001184110) c3(tubulin beta-3 chain isoform 2) c4(75375) c5(75376, 75377, 75378, 75379, 75380, 75381, 75382, 75383) c6( ) c7(sc, T, kh, aNA, if, amp, ha, e, fN, XJ, aA, zN, n, jN, jh, ci, qN, UR, XM, Fc, br, ad, yn, av, anB, ft, aE, lg, cRZ, aT, aJI, rO, dL, K, ae, eL, jf, KG, aJI, Jg, XK, PK, d, hM, A, aW, ff, CRY, aGM, lo, aF, bxw, en, g, t, XI, bDO, bz, dr, pb, cm, bw, es, eM, XG, XO, m, XN, X, XL, L); #c1(TUBB3) c2(NP_006077) c3(tubulin beta-3 chain isoform 1) c4(161714, 161715) c5(161716, 161717, 161718, 161719, 161720, 161721, 161722, 161723, 16/724, 161725, 161726, 16/727, 161728, 161729, 161730, 161731, 161732, 161733) c6( ) c7(sc, T, kh, aNA, if, amp, ha, e, fN, XJ, aA, zN, n, jN, jh, ci, qN, UR, XM, Fc, br, ad, yn, av, anB, ft, aE, lg, cRZ, aT, aJI, rO, dL, K, ae, eL, jf, KG, aJI, Jg, XK, PK, d, hM, A, aW, ff, cRY, aGM, lo, aF, bxw, en, g, t, XI, bDO, bz, dr, pb, cm, bw, es, eM, XG, XO, m, XN, X, XL, L); #c1(TUBB4A) c2(NP_001276056) c3(tubulin beta-4A chain isoform 2) c4(107843) c5( ) c6( ) c7(cZ, T, kh, cSa, qy, fd, fr, V, eA, ad, cSb, Tg, tT, ds, akJ, aT, Wk, aJI, h, dL, dd, he, ae, eL, ud, ael, A, d, aW, Y, eH, Pg, nY, aGM, lo, as, eF, t, QK, bDO, bz, cm, Cv, lV, mx, gZ, aez, eo, m, blu); #c1(TUBB4A) c2(NP_006078) c3(tubulin beta-4A chain isoform 3) c4( ) c5(161734, 161735, 161736, 161737, 161738, 161739, 161740, 161741, 16(742, 161743) c6( ) c7(cZ, T, kh, cSa, qy, fd, fr, V, eA, ad, cSb, Tg, tT, ds, akJ, aT, Wk, aJI, h, dL, dd, he, ae, eL, ul, ael, A, d, aW, Y, eH, Pg, nY, aGM, lo, as, eF, t, QK, bDO, bz, cm, Cv, lV, mx, gZ, aez, eo, m, blu); #c1(TUBB4B) c2(NP_006079) c3(tubulin beta-48 chain) c4(161744) c5(161745, 16/746, 161747, 161748, 161749, 161750) c6( ) c7(bM, T, byb, e, ame, ci, Us, hm, aW, hl, g, cm, qJ, y, bhB, eM, iA, Gs, mx, cj, uJ, zv, m, pw, iu, dt, cV, aj, fr, cd, ai, xC, Mo, ft, i, BR, cU, A, Cw, sp, agF, aPP, CO, jT, ER, aNX, B, aNW, Dw, Rt, cz, tc, AZ, v, V, q, oT, lg, aT, wP, bK, jj, Cy, arl, xR, oV, Y, r, lo, t, arJ, pb, ck, cT, BU, ac, lV, mG, wR, mf, bt, hS, iW, jh, aNH, tT, oW, hD, agm, lQ, mu, d, lx, Gv, xs, cr, dN, S, bz, qa, blZ, bF, es, eL, qe, bE, z, ag, dX, pk, lf, gq, hv); #c1(TUBB6) c2(NP_001290455) c3(tubulin beta-6 chain isoform 3) c4( ) c5(115493, 115494, 115495, 115496, 115497, 115498) c6( ) c7(q, r); #c1(TUBB6) c2(NP_001290456) c3(tubulin beta-6 chain isoform 4) c4( ) c5(115499, 115500, 115501, 115502, 115503) c6( ) c7(q, r); #c1(TUBB6) c2(NP_115914) c3(tubulin beta-6 chain isoform 1) c4(224651, 224652, 224653) c5(224654, 224655, 224656, 224657, 224658, 224659, 224660, 224661, 224662, 224663, 224664, 224665, 224666) c6( ) c7(q, r); #c1(TUBD1) c2(NP_001180538) c3(tubulin delta chain isoform 2) c4(74220) c5(74221, 74222, 74223, 74224, 74225, 74226, 74227) c6( ) c7(aT); #c1(TUBD1) c2(NP_001180539) c3(tubulin delta chain isoform 3) c4(74228, 74229) c5(74230, 74231, 74232, 74233, 74234, 74235) c6( ) c7(aT); #c1(TUBD1) c2(NP_001180540) c3(tubulin delta chain isoform 4) c4(74236) c5(74237, 74238, 74239, 74240, 74241, 74242, 74243, 74244, 74245, 74246, 74247) c6( ) c7(aT); #c1(TUBD1) c2(NP_001180541) c3(tubulin delta chain isoform 5) c4(74248) c5( ) c6( ) c7(aT); #c1(TUBD1) c2(NP_001180542) c3(tubulin delta chain isoform 6) c4( ) c5(74249, 74250, 74251, 74252, 74253, 74254, 74255, 74256, 74257, 74258, 74259) c6( ) c7(aT); #c1(TUBD1) c2(NP_057345) c3(tubulin delta chain isoform 1) c4(190224, 190225) c5(190226, 190227, 190228, 190229, 190230, 190231) c6( ) c7(aT); #c1(TUBD1) c2(XP_005257482) c3(tubulin delta chain isoform X2) c4( ) c5(275152) c6( ) c7(aT); #c1(TUBE1) c2(NP_057346) c3(tubulin epsilon chain) c4(190232, 190233) c5(190234, 190235, 190236, 190237, 190238, 190239, 190240, 190241, 190242, 190243, 190244, 190245) c6( ) c7(A, aT); #c1(TUBE1) c2(XP_011534176) c3(tubulin epsilon chain isoform X1) c4(330473) c5(330474, 330475, 330476, 330477, 330478, 330479, 330480, 330481, 330482, 330483) c6( ) c7(A, aT); #c1(TUBG1) c2(NP_001061) c3(tubulin gamma-1 chain) c4(34497) c5(34498, 34499, 34500, 34501, 34502, 34503) c6( ) c7(PK, aW, FD, aT, zN, cja, cSc, QK); #c1(TUBG2) c2(NP_057521) c3(tubulin gamma-2 chain) c4(191239) c5(191240, 191241, 191242, 191243, 191244, 191245) c6( ) c7(aT); #c1(TUBGCP2) c2(NP_001243546) c3(gamma-tubulin complex component 2 isoform 1) c4(91014) c5(91015, 91016, 91017, 91018, 91019, 91020) c6( ) c7(aZJ, aw); #c1(TUBGCP2) c2(NP_006650) c3(gamma-tubulin complex component 2 isoform 2) c4(166887, 166888, 166889) c5(166890, 166891, 166892, 166893, 166894, 166895, 166896, 166897, 166898, 166899, 166900, 166901, 166902, 166903, 166904, 166905, 166906, 166907, 166908, 166909, 166910, 166911) c6( ) c7(aZJ, aw); #c1(TUBGCP3) c2(NP_006313) c3(gamma-tubulin complex component 3 isoform 1) c4(163915) c5(163916, 163917, 163918, 163919, 163920, 163921) c6( ) c7(aq, y); #c1(TUBGCP4) c2(NP_001273343) c3(gamma-tubulin complex component 4 isoform a) c4( ) c5(104711) c6( ) c7(aT); #c1(TUBGCP4) c2(NP_055259) c3(gamma-tubulin complex component 4 isoform b) c4(180108) c5(180109, 180110, 180111, 180112, 180113, 180114, 180115, 180116, 180117, 180118) c6( ) c7(aT); #c1(TUBGCP4) c2(XP_011519757) c3(gamma-tubulin complex component 4 isoform X3) c4(314449) c5(314450, 314451, 314452, 314453, 314454) c6( ) c7(aT); #c1(TUBGCP5) c2(NP_001096080) c3(gamma-tubulin complex component 5 isoform b) c4(41057) c5(41058, 41059, 41060, 41061, 41062, 41063) c6( ) c7(rc, bN); #c1(TUBGCP5) c2(NP_443135) c3(gamma-tubulin complex component 5 isoform a) c4(229605) c5(229606, 229607, 229608, 229609, 229610, 229611) c6( ) c7(rc, bN); #c1(TUBGCP6) c2(NP_065194) c3(gamma-tubulin complex component 6) c4(204524, 204525, 204526) c5(204527, 204528, 204529, 204530, 204531, 204532, 204533, 204534, 204535, 204536, 204537, 204538, 204539, 204540, 204541, 204542, 204543, 204544, 204545, 204546) c6( ) c7(h, QK, lR); #c1(TUFM) c2(NP_003312) c3(elongation factor Tu, mitochondrial precursor) c4(137369) c5(137370, 137371, 137372, 137373, 137374, 137375) c6( ) c7(bM, yo, dD, ea, ht, cj, cSd, v, eq, hX); #c1(TUFT1) c2(NP_001119809) c3(tuftelin isoform 2) c4(44304, 44305, 44306) c5(44307, 44308, 44309, 44310, 44311, 44312, 44313, 44314, 44315, 44316, 44317, 44318, 44319, 44320, 44321, 44322, 44323, 44324, 44325, 44326, 44327, 44328, 44329, 44330) c6(44331, 44332, 44333, 44334, 44335, 44336) c7(ka, Ud, ki); #c1(TUFT1) c2(NP_001288246) c3(tuftelin isoform 3) c4(113708, 113709) c5(113710, 113711, 113712, 113713, 113714, 113715, 113716, 113717) c6(113718, 113719) c7(ka, Ud, ki); #c1(TUFT1) c2(NP_064512) c3(tuftelin isoform 1) c4(202717) c5(202718, 202719, 202720, 202721, 202722, 202723, 202724, 202725, 202726, 202727, 202728, 202729, 202730, 202731, 202732, 202733, 202734, 202735, 202736, 202737, 202738, 202739, 202740, 202741, 202742, 202743, 202744, 202745, 202746, 202747, 202748, 202749, 202750, 202751, 202752, 202753, 202754, 202755, 202756, 202757, 202758, 202759, 202760, 202761, 202762, 202763, 202764, 202765, 202766, 202767, 202768, 202769, 202770, 202771, 202772, 202773, 202774, 202775, 202776, 202777, 202778, 202779, 202780, 202781, 202782, 202783, 202784, 202785, 202786, 202787, 202788, 202789, 202790, 202791, 202792, 202793, 202794, 202795, 202796, 202797, 202798, 202799, 202800, 202801, 202802, 202803, 202804, 202805, 202806, 202807, 202808, 202809, 202810, 202811, 202812) c6(202813, 202814, 202815) c7(ka, Ud, ki); #c1(TUFT1) c2(XP_011508263) c3(tuftelin isoform X1) c4(298952) c5(298953, 298954, 298955, 298956, 298957, 298958, 298959, 298960) c6(298961, 298962) c7(ka, Ud, ki); #c1(TULP1) c2(NP_001276324) c3(tubby-related protein 1 isoform 2) c4(108018, 108019) c5(108020, 108021, 108022, 108023, 108024, 108025, 108026, 108027, 108028, 108029, 108030, 108031) c6( ) c7(alF, lR, cSf, cwx, cSg, cSe, nw, v, nk, nC); #c1(TULP1) c2(NP_003313) c3(tubby-related protein 1 isoform 1) c4(137376) c5(137377, 137378, 137379, 137380, 137381, 137382, 137383) c6( ) c7(alF, lR, cSf, cwx, cSg, cSe, nw, v, nk, nC); #c1(TULP2) c2(NP_003314) c3(tubby-related protein 2) c4(137384) c5(137385, 137386, 137387, 137388, 137389, 137390, 137391) c6( ) c7(nC, nw, cSe); #c1(TULP3) c2(NP_001153880) c3(tubby-related protein 3 isoform 2) c4(61011) c5(61012, 61013, 61014, 61015, 61016, 61017, 61018) c6( ) c7(kp, eJ, aE); #c1(TULP3) c2(NP_003315) c3(tubby-related protein 3 isoform 1) c4(137392, 137393, 137394) c5(137395, 137396, 137397, 137398, 137399, 137400, 137401, 137402, 137403, 137404, 137405, 137406, 137407, 137408, 137409, 137410, 137411, 137412, 137413, 137414, 137415, 137416) c6( ) c7(kp, eJ, aE); #c1(TULP4) c2(NP_001007467) c3(tubby-related protein 4 isoform 2) c4( ) c5(22708, 22709, 22710, 22711, 22712, 22713) c6( ) c7(bOx); #c1(TULP4) c2(NP_064630) c3(tubby-related protein 4 isoform 1) c4(203389, 203390) c5(203391, 203392, 203393, 203394, 203395, 203396, 203397) c6( ) c7(bOx); #c1(TUSC1) c2(NP_001004125) c3(tumor suppressor candidate gene 1 protein) c4(20441) c5(20442, 20443, 20444, 20445, 20446, 20447) c6( ) c7(dY, pN, hk, Y, aA, cz, aF, hB, bz, ad); #c1(TUSC2) c2(NP_009206) c3(tumor suppressor candidate 2) c4(171498) c5(171499, 171500, 171501, 171502, 171503, 171504) c6( ) c7(T, dA, iW, aj, v, cd, av, ft, ib, aT, d, nO, aW, r, uF, iK, bz, ck, c, eM, Qu, wq, dT, zG, zl, ao); #c1(TUSC3) c2(NP_006756) c3(tumor suppressor candidate 3 isoform a precursor) c4(167738) c5(167739, 167740, 167741, 167742, 167743, 167744, 167745, 167746, 167747, 167748, 167749, 167750, 167751, 167752) c6( ) c7(bM, kh, d, cSh, ff, dv, eH, m, nA, en, eF, t, ad); #c1(TUSC3) c2(NP_839952) c3 (tumor suppressor candidate 3 isoform b precursor) c4(253577) c5(253578, 253579, 253580, 253581, 253582, 253583) c6( ) c7(bM, kh, d, cSh, ff, dv, eH, m, nA, en, eF, t, ad); #c1(TUSC5) c2(NP_758955) c3(tumor suppressor candidate 5) c4(249398) c5(249399, 249400, 249401, 249402, 249403, 249404) c6( ) c7(aj, J); #c1(TUT1) c2(NP_073741) c3(speckle targeted PIP5K1A-regulated poly(A) polymerase) c4(213391, 213392) c5(213393, 213394, 213395, 213396, 213397, 213398, 213399, 213400, 213401, 213402, 213403, 213404) c6( ) c7(T); #c1(TVP23B) c2(NP_057162) c3(Golgi apparatus membrane protein TVP23 homolog B) c4(189224) c5(189225, 189226, 189227, 189228, 189229, 189230) c6( ) c7(aC); #c1(TWF1) c2(NP_001229326) c3(twinfilin-1 isoform 1) c4(84206) c5(84207, 84208, 84209, 84210, 84211, 84212) c6( ) c7(aW, jh, aT); #c1(TWF1) c2(NP_002813) c3(twinfilin-1 isoform 2) c4(132405, 132406) c5(132407, 132408, 132409, 132410, 132411, 132412, 132413, 132414, 132415, 132416, 132417, 132418) c6( ) c7(aW, jh, aT); #c1(TWIST1) c2(XP_011513798) c3(twist-related protein 1 isoform X1) c4(306263) c5(306264, 306265, 306266, 306267, 306268, 306269, 306270) c6( ) c7(gh, T, aC, rV, if, bB, ci, zW, asb, aE, bnt, K, hu, eL, aa, aW, are, Az, btB, agj, g, eF, cm, bw, zV, eM, nB, m, bsB, zt, ht, aBx, n, nA, qN, cd, br, ai, agi, By, dL, Dz, cSj, btj, dB, btW, hL, zG, art, it, B, fG, aA, v, AO, q, eA, aT, rz, ae, bA, jr, avg, s, Y, r, en, cl, t, iK, mG, np, yH, cSk, cSi, auq, zU, pz, xt, ad, av, hd, yt, hP, fb, aca, ff, eH, dq, aeV, aF, aJW, iV, bz, aJI, LB, eL, bE, uR, iu, sH); #c1(TWIST2) c2(NP_001258822) c3(twist-related protein 2) c4(96563) c5(96564, 96565, 96566, 96567, 96568, 96569) c6( ) c7(T, asW, if, ci, qN, q, cSl, x, rC, aT, iL, B, aW, ff, eH, r, en, eF, t, Ci, ck, cj, m, k, aAE); #c1(TWSG1) c2(NP_065699) c3(twisted gastrulation protein homolog 1 precursor) c4(204997) c5(204998, 204999, 205000, 205001, 205002, 205003) c6( ) c7(T, id, if, al, KY, aA, bB, n, fr, ci, qN, q, ad, x, av, ft, ib, aE, py, aT, gs, K, ZW, ae, jf, aW, Y, ff, r, aF, en, g, t, cm, bz, gk, Ew, bP, eM, bE, afd, hR, aSp, m, bt, it, B, VB); #c1(TXK) c2(NP_003319) c3(tyrosine-protein kinase TXK) c4(137457) c5(137458, 137459, 137460, 137461, 137462, 137463) c6( ) c7(hH); #c1(TXLNG) c2(NP_001162154) c3(gamma-taxilin isoform 2) c4(66338, 66339) c5(66340, 66341, 66342, 66343, 66344, 66345, 66346, 66347, 66348, 66349, 66350, 66351, 66352, 66353, 66354, 66355) c6( ) c7(aq, B); #c1(TXLNG) c2(NP_060830) c3(gamma-taxilin isoform 1) c4(198027, 198028, 198029, 198030) c5(198031, 198032, 198033, 198034, 198035, 198036, 198037) c6( ) c7(aq, B); #c1(TXLNG) c2(XP_005274615) c3(gamma-taxilin isoform X2) c4(285215, 285216) c5(285217, 285218, 285219, 285220, 285221, 285222, 285223, 285224, 285225, 285226, 285227, 285228, 285229, 285230, 285231, 285232, 285233, 285234, 285235, 285236, 285237, 285238, 285239, 285240, 285241, 285242, 285243, 285244) c6( ) c7(aq, B); #c1(TXLNG) c2(XP_011543845) c3(gamma-taxilin isoform X1) c4( ) c5(342986) c6( ) c7(aq, B); #c1(TXN) c2(NP_001231867) c3(thioredoxin isoform 2) c4(87585) c5(87586, 87587, 87588, 87589, 87590, 87591) c6( ) c7(T, os, aC, ea, e, eb, bB, fd, ci, aR, aE, Bc, K, aiV, Pf, aW, fc, hl, zC, as, eF, cm, eM, aCT, wE, ki, vA, U, cj, m, dD, gU, aw, fN, BH, aj, PJ, JW, cd, ai, ke, b, cc, kH, cU, lG, OS, A, dw, op, aDu, B, bL, aA, sM, cz, q, J, eA, uT, lg, aT, cy, adV, bq, ae, eu, Y, afS, cSm, r, t, f, iK, tR, ck, iM, ac, c, wR, dy, ao, hS, yv, oZ, dG, eq, wV, ad, dP, av, yt, ds, ji, MG, d, je, dq, aF, bz, dr, qa, Hp, D, bE, apv, i, hE, y); #c1(TXN) c2(NP_003320) c3(thioredoxin isoform 1) c4(137464) c5(137465, 137466, 137467, 137468, 137469, 137470) c6( ) c7(T, os, aC, ea, e, eb, bB, fd, ci, aR, aE, Bc, K, aiV, Pf, aW, fc, hl, zC, as, eF, cm, eM, aCT, wE, ki, vA, U, cj, m, dD, gU, aw, fN, BH, aj, PJ, JW, cd, ai, ke, b, cc, kH, cU, lG, oS, A, dw, op, aDu, B, bL, aA, sM, cz, q, J, eA, uT, lg, aT, cy, adV, bq, ae, eu, Y, afS, cSm, r, t, f, iK, tR, ck, iM, ac, c, wR, dy, ao, hS, yv, oZ, dG, eq, wV, ad, dP, av, yt, ds, ji, MG, d, je, dq, aF, bz, dr, qa, Hp, D, bE, apv, i, hE, y); #c1(TXN2) c2(XP_005261565) c3(thioredoxin, mitochondrial isoform X2) c4( ) c5(277481, 277482) c6( ) c7(BN, lp, Bc, aW, eH, mx, aT, lG, aj, hy, K, C, bq, eF, ne, J); #c1(TXN2) c2(XP_006724289) c3(thioredoxin, mitochondrial isoform X1) c4(295888) c5(295889, 295890, 295891, 295892, 295893, 295894, 295895, 295896) c6( ) c7(BN, lp, Bc, aW, eH, mx, aT, lG, aj, hy, K, C, bq, eF, ne, J); #c1(TXNDC15) c2(NP_078991) c3(thioredoxin domain-containing protein 15 precursor) c4(216438, 216439, 216440) c5(216441, 216442, 216443, 216444, 216445, 216446, 216447, 216448, 216449, 216450, 216451, 216452, 216453, 216454, 216455, 216456, 216457, 216458) c6( ) c7(aW, fd, aT); #c1(TXNDC16) c2(NP_065835) c3(thioredoxin domain-containing protein 16 isoform 1 precursor) c4(205766) c5(205767, 205768, 205769, 205770, 205771, 205772, 205773, 205774) c6(205775, 205776) c7(Na, ip); #c1(TXNDC17) c2(NP_116120) c3(thioredoxin domain-containing protein 17) c4(225421) c5(225422, 225423, 225424, 225425, 225426, 225427) c6( ) c7(it); #c1(TXNDC5) c2(NP_001139021) c3(thioredoxin domain-containing protein 5 isoform 3) c4(58675) c5(58676, 58677, 58678, 58679, 58680) c6( ) c7(T, y, mR, aE, Jl, fO, NT, aT, aA, aq, fd, aF, qj, K, qN, iR, AD, B, ad, pC); #c1(TXNDC5) c2(NP_110437) c3(thioredoxin domain-containing protein 5 isoform 1 precursor) c4(220368, 220369) c5(220370, 220371, 220372, 220373, 220374, 220375, 220376, 220377, 220378, 220379, 220380, 220381, 220382) c6( ) c7(T, y, mR, aE, Jl, FO, NT, aT, aA, aq, fd, aF, qj, K, qN, iR, AO, B, ad, pC); #c1(TXNL1) c2(NP_004777) c3(thioredoxin-like protein 1) c4(149533, 149534) c5(149535, 149536, 149537, 149538, 149539, 149540, 149541, 149542, 149543, 149544, 149545, 149546) c6( ) c7(bt, aF, K, aA); #c1(TXNRD1) c2(NP_001087240) c3(thioredoxin reductase 1, cytoplasmic isoform 3) c4(38268, 38269, 38270, 38271, 38272, 38273, 38274, 38275, 38276, 38277, 38278, 38279, 38280, 38281, 38282, 38283, 38284) c5(38285, 38286, 38287, 38288, 38289, 38290, 38291, 38292, 38293, 38294, 38295, 38296, 38297, 38298, 38299, 38300, 38301, 38302) c6( ) c7(bM, T, eR, WA, aw, ha, ol, bB, bZ, aj, Vd, j, v, PJ, nE, aR, J, ad, qC, fU, HF, av, ib, yQ, hb, oj, adX, al, IG, Ak, bK, K, Gz, bq, hS, jj, ae, ajU, bA, ak, DA, kl, s, aW, aM, az, hl, ff, So, dq, HL, en, Z, f, px, bz, BE, dr, bN, iK, ck, mO, adc, c, adZ, SE, dB, bE, awN, eM, bo, U, avF, eL, anx, lM, y, kS, ao, DB); #c1(TXNRD1) c2(NP_001248374) c3(thioredoxin reductase 1, cytoplasmic isoform 4) c4( ) c5(93836, 93837, 93838, 93839, 93840, 93841, 93842, 93843, 93844, 93845, 93846, 93847, 93848, 93849, 93850, 93851, 93852, 93853, 93854, 93855, 93856, 93857, 93858, 93859, 93860, 93861, 93862, 93863, 93864, 93865, 93866, 93867, 93868, 93869, 93870, 93871, 93872, 93873, 93874, 93875, 93876, 93877, 93878, 93879, 93880, 93881, 93882, 93883, 93884, 93885, 93886, 93887, 93888, 93889, 93890, 93891, 93892) c6( ) c7(bM, T, eR, WA, aw, ha, ol, bB, bZ, aj, Vd, j, v, PJ, nE, aR, J, ad, qC, fU, HF, av, ib, yl, hb, oj, adX, aT, lG, Ak, bK, K, Gz, bq, hS, jj, ae, ajU, bA, ak, DA, kl, s, aW, aM, az, hl, ff, So, dq, HL, en, Z, f, px, bz, eE, dr, bN, iK, ck, mO, adc, c, adZ, SE, dB, bE, awN, eM, bo, U, avF, eL, anx, lM, y, kS, ao, DB); #c1(TXNRD1) c2(NP_001248375) c3(thioredoxin reductase 1, cytoplasmic isoform 5) c4( ) c5(93893, 93894, 93895, 93896, 93897) c6( ) c7(bM, T, eR, WA, aw, ha, ol, bB, bZ, aj, Vd, j, v, PJ, nE, aR, J, ad, qC, fU, HF, av, ib, yQ, hb, oj, adX, aT, lG, Ak, bK, K, Gz, bq, hS, jj, ae, ajU, bA, ak, DA, kl, s, aW, aM, az, hl, ff, So, dq, HL, en, Z, f, px, bz, eE, dr, bN, iK, ck, mO, adc, c, adZ, SE, dB, bE, awN, eM, bo, U, avF, eL, anx, lM, y, kS, ao, DB); #c1(TXNRD1) c2(NP_003321) c3(thioredoxin reductase 1, cytoplasmic isoform 1) c4(137471, 137472) c5(137473, 137474, 137475, 137476, 137477, 137478, 137479, 137480, 137481, 137482, 137483, 137484) c6( ) c7(bM, T, eR, WA, aw, ha, ol, bB, bZ, aj, Vd, j, v, PJ, nE, aR, J, ad, qC, fU, HF, av, ib, yQ, hb, oj, adX, aT, lG, Ak, bK, K, Gz, bq, hS, jj, ae, ajU, bA, ak, DA, kl, s, aW, aM, az, hl, ff, So, dq, HL, en, Z, f, px, bz, eE, dr, bN, iK, ck, mO, adc, c, adZ, SE, dB, bE, awN, eM, bo, U, avF, eQ, anx, lM, y, KS, ao, DB); #c1(TXNRD1) c2(NP_877420) c3(thioredoxin reductase 1, cytoplasmic isoform 2) c4( ) c5(256765, 256766, 256767, 256768, 256769, 256770, 256771, 256772, 256773, 256774, 256775, 256776, 256777, 256778, 256779, 256780, 256781, 256782, 256783, 256784, 256785, 256786, 256787, 256788, 256789) c6( ) c7(bM, T, eR, WA, aw, ha, ol, bB, bZ, aj, Vd, j, v, PJ, nE, aR, J, ad, qC, fU, HF, av, ib, yQ, hb, oj, adX, aT, lG, Ak, bK, K, Gz, bq, hS, jj, ae, ajU, bA, ak, DA, kl, s, aW, aM, az, hl, ff, So, dq, HL, en, Z, f, px, bz, eE, dr, bN, iK, ck, mO, adc, c, adZ, SE, dB, bE, awN, eM, bo, U, avF, eL, anx, lM, y, kS, ao, DB); #c1(TXNRD2) c2(NP_001269441) c3(thioredoxin reductase 2, mitochondrial isoform 2 precursor) c4(101669) c5(101670, 101671, 101672, 101673, 101674, 101675) c6( ) c7(oq, T, Js, zt, aA, hz, gf, qj, q, ad, md, hd, no, aT, lG, dL, K, bq, ae, ak, aW, r, aF, ahV, pv, y, aZT, nB, kz, ali, hr, mx, dX, gT, B, Jo, pC); #c1(TXNRD2) c2(NP_006431) c3(thioredoxin reductase 2, mitochondrial isoform 1 precursor) c4(165155, 165156, 165157, 165158, 165159, 165160) c5(165161, 165162, 165163, 165164, 165165, 165166, 165167, 165168, 165169, 165170, 165171, 165172, 165173, 165174, 165175, 165176, 165177, 165178, 165179, 165180, 165181, 165182, 165183, 165184, 165185, 165186, 165187, 165188, 165189, 165190, 165191, 165192, 165193, 165194, 165195) c6( ) c7(oq, T, Js, zt, aA, hz, gf, qj, q, ad, md, hd, no, aT, lG, dL, K, bq, ae, ak, aW, r, aF, ahV, pv, y, aZT, nB, kz, ali, hr, mx, dX, gT, B, Jo, pC); #c1(TXNRD3) c2(NP_001166984) c3(thioredoxin reductase 3 isoform 2) c4(68757, 68758) c5(68759, 68760, 68761, 68762, 68763, 68764, 68765, 68766, 68767, 68768) c6( ) c7(uH, e, aw, cz, cy, aj, sl, ae, J, ad); #c1(TXNRD3) c2(NP_443115) c3(thioredoxin reductase 3 isoform 1) c4(229498) c5(229499, 229500, 229501, 229502, 229503, 229504, 229505) c6( ) c7(uH, e, aw, cz, cy, aj, sl, ae, J, ad); #c1(TXNRD3NB) c2(NP_001034872) c3(protein TXNRD3NB) c4(32371) c5(32372, 32373, 32374, 32375, 32376, 32377, 32378, 32379, 32380, 32381, 32382) c6( ) c7(cr); #c1(TYK2) c2(XP_011526547) c3(non-receptor tyrosine-protein kinase TYK2 isoform X1) c4(320848) c5(320849, 320850, 320851, 320852, 320853, 320854, 320855, 320856, 320857, 320858) c6( ) c7(bL, gV, T, bM, Dw, al, bO, jh, bS, v, fr, V, mC, ad, bhq, ip, F, aT, Ds, hn, DU, btE, fb, bc, ul, A, aW, oV, cr, eZ, dN, cu, lo, pm, t, bw, cSn, btk, ck, pc, iT, nI, pg, fL, y, bt, zA); #c1(TYK2) c2(XP_011526549) c3(non-receptor tyrosine-protein kinase TYK2 isoform X2) c4(320859) c5(320860, 320861, 320862, 320863, 320864, 320865) c6( ) c7(bL, gV, T, bM, Dw, al, bO, jh, bS, v, fr, V, mC, ad, bhq, ip, F, aT, Ds, hn, DU, btE, fb, bc, ul, A, aW, oV, cr, eZ, dN, cu, lo, pm, t, bw, cSn, btK, ck, pc, iT, nL, pg, fL, y, bt, zA); #c1(TYK2) c2(XP_011526550) c3(non-receptor tyrosine-protein kinase TYK2 isoform X3) c4(320866) c5( ) c6( ) c7(bL, gV, T, bM, Dw, al, bO, jh, bS, v, fr, V, mC, ad, bhq, ip, F, aT, Ds, hn, DU, btE, fb, bc, uQ, A, aW, oV, cr, eZ, dN, cu, lo, pm, t, bw, cSn, btk, ck, pc, iT, nI, pg, fL, y, bt, zA); #c1(TYK2) c2(XP_011526554) c3(non-receptor tyrosine-protein kinase TYK2 isoform X7) c4( ) c5(320867, 320868, 320869, 320870, 320871, 320872) c6( ) c7(bL, gV, T, bM, Dw, al, bO, jh, bS, v, fr, V, mC, ad, bhq, ip, F, aT, Ds, hn, DU, btE, fb, bc, ul, A, aW, oV, cr, eZ, dN, cu, lo, pm, t, bw, cSn, btK, ck, pc, iT, nI, pg, fL, y, bt, ZA); #c1(TYMP) c2(NP_001107227) c3(thymidine phosphorylase isoform 1 proprotein) c4(42814) c5(42815, 42816, 42817, 42818, 42819, 42820, 42821, 42822, 42823, 42824, 42825, 42826, 42827, 42828) c6( ) c7(bL, T, dD, aC, KR, ea, if, al, NT, cSp, aA, bB, n, aj, aNG, j, ci, fu, bX, eq, qN, q, ad, eA, dP, hd, aE, cSo, hb, afQ, aT, Nf, dL, K, dF, eL, Ft, xQ, d, s, aW, Y, ev, ff, cr, r, ks, af, brr, en, avG, GF, t, cM, kC, bz, Px, cl, g, cm, ar, mO, sS, nM, Fr, c, zr, bE, hk, eM, X, gX, hL, m, lM, y, it, B, Al); #c1(TYMS) c2(NP_001062) c3(thymidylate synthase) c4(34504, 34505, 34506) c5(34507, 34508, 34509, 34510, 34511, 34512, 34513, 34514, 34515, 34516, 34517, 34518, 34519, 34520, 34521, 34522, 34523, 34524) c6( ) c7(ng, G, T, aC, pf, fV, if, fd, bB, ci, aR, ip, x, Na, aE, iI, fS, pX, dV, ja, azm, K, hu, hH, eL, cSq, ln, Jl, aW, CT, LZ, NR, g, eF, GR, fT, eM, iA, lp, wE, k, m, iu, sw, ht, NT, aip, n, oY, awr, fr, ct, qN, zP, eW, ft, qg, gd, h, Gw, hn, dL, wb, ey, ask, hi, MX, AQ, ahJ, aq, YF, zZ, cM, ar, bP, gY, Qu, hL, bm, at, bD, Qz, Pr, B, it, fM, atB, fZ, aA, hy, cSr, j, bX, q, hb, qK, yg, aT, bSx, Nc, Tj, ae, ne, eu, Uf, s, nO, iU, Y, ev, aZs, apk, r, en, aDY, cl, t, f, iK, ck, Qi, ac, aGi, nG, gX, dy, ij, byK, aws, bt, gV, GI, Dd, iW, jh, bS, uu, hF, ad, afb, av, ib, py, NS, oW, iL, WW, d, hG, ff, eZ, aF, avG, Z, bz, iq, es, bE, cgA, Gu, i, y, X); #c1(TYR) c2(NP_000363) c3(tyrosinase precursor) c4(8024) c5(8025, 8026, 8027, 8028, 8029, 8030, 8031, 8032, 8033, 8034, 8035, 8036, 8037, 8038, 8039, 8040, 8041, 8042, 8043, 8044, 8045, 8046, 8047, 8048, 8049, 8050, 8051, 8052, 8053, 8054, 8055, 8056, 8057, 8058, 8059, 8060, 8061, 8062, 8063, 8064, 8065, 8066, 8067, 8068, 8069, 8070, 8071, 8072, 8073, 8074, 8075, 8076, 8077, 8078, 8079, 8080, 8081, 8082, 8083, 8084, 8085, 8086, 8087, 8088, 8089, 8090, 8091, 8092, 8093, 8094, 8095, 8096, 8097, 8098, 8099, 8100, 8101, 8102, 8103, 8104, 8105, 8106, 8107, 8108, 8109, 8110, 8111, 8112, 8113, 8114, 8115, 8116, 8117) c6(8118) c7(T, qj, pC, cSw, YD, aJO, alx, K, cp, Ls, HA, XC, bHy, cm, iP, eM, aBO, bbF, m, gn, cZ, n, rc, eW, Nn, azt, kH, dL, cSv, dd, he, awL, MX, kj, cSx, bP, cA, dB, hk, at, JJ, cSu, yk, ze, oc, V, eA, cSt, afQ, bK, an, ae, Y, yL, cSs, en, bu, t, bN, ck, sS, iM, Dy, cer, rk, ao, hl, iW, cSz, ad, cmw, Rq, ds, cb, atX, d, je, asf, ff, cr, eZ, gl, cSy, aTa, dr, bE, lF, bC, aYn); #c1(TYRO3) c2(NP_006284) c3(tyrosine-protein kinase receptor TYRO3 precursor) c4(163592) c5( ) c6( ) c7(bM, T, dD, cSA, Bm, eq, ai, mC, ad, av, hW, h, cy, ae, eG, Ls, eL, lo, pL, bz, lt, ck, cS, fL, QC, bt); #c1(TYRO3) c2(XP_011520294) c3(tyrosine-protein kinase receptor TYRO3 isoform X1) c4( ) c5(315130, 315131, 315132, 315133, 315134, 315135, 315136) c6( ) c7(bM, T, dD, cSA, Bm, eq, ai, mC, ad, av, hW, h, cy, ae, eG, Ls, eZ, lo, pL, bz, Qt, ck, cS, fL, QC, bt); #c1(TYRO3) c2(XP_011520295) c3(tyrosine-protein kinase receptor TYRO3 isoform X2) c4(315137) c5(315138, 315139, 315140, 315141, 315142) c6( ) c7(bM, T, dD, cSA, Bm, eq, ai, mC, ad, av, hW, h, cy, ae, eG, Ls, eL, lo, pL, bz, Qt, ck, cS, fL, QC, bt); #c1(TYROBP) c2(NP_001166985) c3(TYRO protein tyrosine kinase-binding protein isoform 3 precursor) c4(68769) c5(68770, 68771, 68772, 68773, 68774, 68775) c6( ) c7(y, hl, abg, eZ, amW, he, dy, axQ, ey, mF, t, Fc, bOF, dr, Hp); #c1(TYROBP) c2(NP_003323) c3(TYRO protein tyrosine kinase-binding protein isoform 1 precursor) c4(137485) c5(137486, 137487, 137488, 137489, 137490, 137491) c6( ) c7(y, hl, abg, eZ, amW, he, dy, axQ, ey, mF, t, Fc, bOF, dr, Hp); #c1(TYROBP) c2(NP_937758) c3(TYRO protein tyrosine kinase-binding protein isoform 2 precursor) c4(259050) c5(259051, 259052, 259053, 259054, 259055, 259056, 259057, 259058, 259059, 259060) c6( ) c7(y, hl, abg, eZ, amW, he, dy, axQ, ey, mF, t, Fc, bOF, dr, Hp); #c1(TYRP1) c2(NP_000541) c3(5,6-dihydroxyindole-2-carboxylic acid oxidase precursor) c4(12405, 12406, 12407) c5(12408, 12409, 12410, 12411, 12412, 12413, 12414, 12415, 12416, 12417, 12418, 12419, 12420, 12421, 12422, 12423, 12424, 12425) c6( ) c7(bM, T, ZV, lT, e, cSz, cV, cbc, bB, jN, aNG, uu, qj, lN, V, aYs, pC, ad, aJO, av, xy, cSt, ec, afd, aT, hD, vw, cmx, HR, K, ae, ajU, cp, kj, aW, Y, Gv, ff, hl, XC, FU, en, bHy, dw, t, vo, g, cm, dr, iP, cSB, Dy, GU, KB, lI, hk, bE, wB, U, lF, nw, dy, cSC, m, gn, eo, L); #c1(TYSND1) c2(NP_001035363) c3(peroxisomal leader peptide-processing protease isoform b) c4(32815) c5(32816, 32817, 32818, 32819, 32820, 32821, 32822) c6( ) c7(gU, al); #c1(TYSND1) c2(NP_775826) c3(peroxisomal leader peptide-processing protease isoform a) c4(250446) c5(250447, 250448, 250449, 250450, 250451, 250452) c6( ) c7(gU, al); #c1(TYW1B) c2(XP_011514532) c3(S-adenosyl-L-methionine-dependent tRNA 4-demethylwyosine synthase isoform X1) c4(307233) c5( ) c6( ) c7(ac, an); #c1(TYW1B) c2(XP_011514534) c3(S-adenosyl-L-methionine-dependent tRNA 4-demethylwyosine synthase isoform X3) c4(307234) c5(307235, 307236, 307237, 307238, 307239, 307240, 307241, 307242, 307243, 307244) c6( ) c7(ac, an); #c1(UZAF1) c2(NP_001020375) c3(splicing factor UZAF 35 kDa subunit isoform c) c4( ) c5(28685, 28686, 28687, 28688, 28689, 28690, 28691, 28692) c6( ) c7(pq, T, cPs, aE, yR, cj, cz, h, bw, yZ, np, ci, g, hf, yU, f, bt, br, bA); #c1(UZAF2) c2(NP_001012496) c3(splicing factor UZAF 65 kDa subunit isoform b) c4(25012) c5(25013, 25014, 25015, 25016, 25017, 25018) c6( ) c7(q, r); #c1(UZAF2) c2(NP_009210) c3(splicing factor UZAF 65 kDa subunit isoform a) c4(171532) c5(171533, 171534, 171535, 171536, 171537, 171538) c6( ) c7(q, r); #c1(UACA) c2(NP_001008225) c3(uveal autoantigen with coiled-coil domains and ankyrin repeats isoform 2) c4(22916, 22917) c5(22918, 22919, 22920, 22921, 22922, 22923, 22924, 22925, 22926, 22927, 22928, 22929) c6( ) c7(eM, T, B, mP, bX); #c1(UACA) c2(NP_060473) c3(uveal autoantigen with coiled-coil domains and ankyrin repeats isoform 1) c4(196117) c5(196118, 196119, 196120, 196121, 196122, 196123) c6( ) c7(eM, T, B, mP, bX); #c1(UACA) c2(XP_005254586) c3(uveal autoantigen with coiled-coil domains and ankyrin repeats isoform X4) c4(273265) c5(273266, 273267, 273268, 273269, 273270, 273271) c6( ) c7(eM, T, B, mP, bX); #c1(UAP1) c2(NP_003106) c3(UDP-N-acetylhexosamine pyrophosphorylase) c4(135363) c5(135364, 135365, 135366, 135367, 135368, 135369, 135370, 135371, 135372, 135373, 135374, 135375) c6( ) c7(cz); #c1(UAP1) c2(XP_005245522) c3(UDP-N-acetylhexosamine pyrophosphorylase isoform X2) c4(266690) c5(266691, 266692, 266693, 266694, 266695, 266696) c6( ) c7(cz); #c1(UAP1) c2(XP_011508217) c3(UDP-N-acetylhexosamine pyrophosphorylase isoform X1) c4(298827, 298828) c5(298829, 298830, 298831, 298832, 298833, 298834, 298835, 298836, 298837, 298838) c6( ) c7(cz); #c1(UBA1) c2(XP_011542255) c3(ubiquitin-like modifier-activating enzyme 1 isoform X2) c4(340792, 340793, 340794, 340795, 340796) c5(340797, 340798, 340799, 340800, 340801, 340802, 340803, 340804, 340805, 340806, 340807, 340808, 340809, 340810) c6( ) c7(Qi, apq, apl, kf, cSD, arX, y); #c1(UBA1) c2(XP_011542256) c3(ubiquitin-like modifier-activating enzyme 1 isoform X3) c4(340811) c5(340812, 340813, 340814, 340815, 340816, 340817, 340818, 340819, 340820, 340821, 340822, 340823) c6( ) c7(Qi, apq, apl, kf, cSD, arX, y); #c1(UBA1) c2(XP_011542257) c3(ubiquitin-like modifier-activating enzyme 1 isoform X4) c4(340824) c5( ) c6( ) c7(Qi, apq, apl, kf, cSD, arX, y); #c1(UBA1) c2(XP_011542258) c3(ubiquitin-like modifier-activating enzyme 1 isoform X1) c4( ) c5(340825, 340826, 340827, 340828, 340829, 340830, 340831, 340832, 340833, 340834, 340835, 340836, 340837, 340838, 340839, 340840, 340841, 340842, 340843, 340844) c6( ) c7(Qi, apq, apl, kf, cSD, arX, y); #c1(UBA2) c2(NP_005490) c3(SUMO-activating enzyme subunit 2) c4(156752) c5(156753, 156754, 156755, 156756, 156757, 156758) c6( ) c7(ahs, ahp, WO, FB, nd, aeq, ha, aT, nf, al, cC, aht, lm, h, nA, ahu, FD, xw, aR, bN); #c1(UBA2) c2(XP_005258461) c3(SUMO-activating enzyme subunit 2 isoform X1) c4( ) c5(275680) c6( ) c7(ahs, ahp, WO, FB, nd, aeq, ha, aT, nf, aJI, cC, aht, lm, h, nA, ahu, FD, xw, aR, bN); #c1(UBA2) c2(XP_006723025) c3(SUMO-activating enzyme subunit 2 isoform X3) c4(294987, 294988) c5(294989, 294990, 294991, 294992, 294993, 294994, 294995, 294996, 294997, 294998, 294999) c6( ) c7(ahs, ahp, WO, FB, nd, aeq, ha, aT, nf, aJI, cC, aht, lm, h, nA, ahu, FD, xw, aR, bN); #c1(UBA2) c2(XP_011524606) c3(SUMO-activating enzyme subunit 2 isoform X2) c4( ) c5(319149) c6( ) c7(ahs, ahp, WO, FB, nd, aeq, ha, aT, nf, aJI, cC, aht, lm, h, nA, ahu, FD, xw, aR, bN); #c1(UBA3) c2(NP_003959) c3(NEDD8-activating enzyme E1 catalytic subunit isoform 1) c4(142465) c5(142466, 142467, 142468, 142469, 142470, 142471) c6( ) c7(f); #c1(UBA3) c2(NP_937838) c3(NEDD8-activating enzyme E1 catalytic subunit isoform 2) c4(259265) c5(259266, 259267, 259268, 259269, 259270, 259271) c6( ) c7(f); #c1(UBA3) c2(XP_011532512) c3(NEDD8-activating enzyme E1 catalytic subunit isoform X1) c4(328634) c5( ) c6( ) c7(f); #c1(UBA3) c2(XP_011532513) c3(NEDD8-activating enzyme E1 catalytic subunit isoform X2) c4( ) c5(328635, 328636, 328637, 328638, 328639, 328640) c6( ) c7(f); #c1(UBA7) c2(NP_003326) c3(ubiquitin-like modifier-activating enzyme 7) c4(137492) c5(137493, 137494, 137495, 137496, 137497, 137498) c6( ) c7(av, T, aoT, lo, jj, bz, kS, pi); #c1(UBAC1) c2(NP_057256) c3 (ubiquitin-associated domain-containing protein 1) c4(189746) c5(189747, 189748, 189749, 189750, 189751, 189752) c6( ) c7(C); #c1(UBAC2) c2(NP_001137544) c3(ubiquitin-associated domain-containing protein 2 isoform 1 precursor) c4(56014) c5(56015, 56016, 56017, 56018, 56019, 56020) c6( ) c7(hH, sS); #c1(UBAC2) c2(NP_808882) c3(ubiquitin-associated domain-containing protein 2 isoform 2) c4(253002) c5(253003, 253004, 253005, 253006, 253007, 253008) c6( ) c7(hH, sS); #c1(UBAP1) c2(NP_001164672) c3(ubiquitin-associated protein 1 isoform 2) c4(67168) c5(67169, 67170, 67171, 67/72, 67173, 67174) c6( ) c7(WY, aw, rS, aaE, ci, hm); #c1(UBAP1) c2(NP_001164673) c3(ubiquitin-associated protein 1 isoform 3) c4(67/75) c5(67176, 67177, 67178, 67179, 67180, 67181) c6( ) c7(WY, aw, rS, aaE, ci, hm); #c1(UBAP1) c2(NP_057609) c3(ubiquitin-associated protein 1 isoform 1) c4(191721, 19/722) c5(191723, 191724, 191725, 191726, 191727, 19/728, 191729, 191730, 191731, 191732, 191733, 191734, 191735, 191736, 191737, 191738, 191739, 191740, 191741, 191742, 191743, 191744, 191745, 191746) c6( ) c7(WY, aw, rS, aaE, ci, hm); #c1(UBAP2) c2(NP_001269458) c3(ubiquitin-associated protein 2 isoform 4) c4(101707, 101708, 101709) c5(101710, 101711, 101712, 101713, 101714, 101715, 101716, 101717, 101718, 101719, 101720, 101721) c6( ) c7(WY); #c1(UBAP2) c2(NP_001269459) c3(ubiquitin-associated protein 2 isoform 5) c4( ) c5(101722, 101723, 101724, 101725, 101726, 101727, 101728, 101729, 101730, 101731, 101732, 101733) c6( ) c7(WY); #c1(UBAP2) c2(NP_060919) c3(ubiquitin-associated protein 2 isoform 1) c4(198635, 198636, 198637, 198638, 198639, 198640, 198641, 198642) c5(198643, 198644, 198645, 198646, 198647, 198648, 198649, 198650, 198651, 198652, 198653, 198654, 198655, 198656, 198657, 198658, 198659, 198660, 198661, 198662, 198663, 198664, 198665, 198666, 198667, 198668, 198669, 198670, 198671, 198672, 198673, 198674, 198675, 198676, 198677, 198678, 198679, 198680, 198681, 198682, 198683, 198684, 198685, 198686, 198687, 198688, 198689, 198690, 198691, 198692) c6( ) c7(WY); #c1(UBASH3A) c2(NP_001001895) c3(ubiquitin-associated and SH3 domain-containing protein A isoform 2) c4(19135) c5(19136, 19137, 19138, 19139, 19140, 19141) c6( ) c7(A, Y, eZ, hD, cL, v, hp, aj, qj, y, J); #c1(UBASH3A) c2(NP_001230396) c3(ubiquitin-associated and SH3 domain-containing protein A isoform 3) c4(86274) c5(86275, 86276, 86277, 86278, 86279, 86280) c6( ) c7(A, Y, eZ, hD, cL, v, hp, aj, qj, y, J); #c1(UBASH3A) c2(NP_061834) c3(ubiquitin-associated and SH3 domain-containing protein A isoform 1) c4(200695) c5(200696, 200697, 200698, 200699, 200700, 200701) c6( ) c7(A, Y, eZ, hD, cL, v, hp, aj, qj, y, J); #c1(UBASH3A) c2(XP_011527907) c3 (ubiquitin-associated and SH3 domain-containing protein A isoform X2) c4(323003) c5( ) c6( ) c7(A, Y, eZ, hD, cL, v, hp, aj, qj, y, J); #c1(UBASH3A) c2(XP_011527911) c3 (ubiquitin-associated and SH3 domain-containing protein A isoform X4) c4( ) c5(323004, 323005, 323006, 323007, 323008, 323009) c6( ) c7(A, Y, eZ, HD, cl, v, hp, aj, qj, y, J); #c1(UBASH3B) c2(NP_116262) c3(ubiquitin-associated and SH3 domain-containing protein B) c4(226020) c5(226021, 226022, 226023, 226024, 226025, 226026) c6( ) c7(bL, bM, hl, Xi, n, mC, ad, x, aT, Xh, hH, ae, ul, mR, iI, aW, eZ, aq, Xj, iR, ac, Vy, k, fL, ym, bt); #c1(UBB) c2(NP_001268647) c3(polyubiquitin-B precursor) c4(100002, 100003, 100004, 100005, 100006, 100007, 100008) c5(100009, 100010, 100011, 100012, 100013, 100014, 100015, 100016, 100017, 100018, 100019, 100020, 100021, 100022, 100023, 100024, 100025, 100026, 100027, 100028, 100029, 100030, 100031, 100032, 100033, 100034, 100035, 100036, 100037, 100038, 100039, 100040, 100041, 100042, 100043, 100044, 100045, 100046, 100047, 100048, 100049, 100050, 100051, 100052, 100053, 100054, 100055, 100056, 100057, 100058, 100059, 100060, 100061, 100062, 100063, 100064, 100065, 100066, 100067, 100068, 100069, 100070) c6( ) c7(T, if, GC, qN, Hx, Dq, alD, H, af, ak, adF, lb, bSc, aaO, f, cm, axG, bb, cBd, U, act, aJI, L); #c1(UBC) c2(NP_066289) c3(polyubiquitin-C) c4(207043, 207044, 207045, 207046) c5(207047, 207048, 207049, 207050, 207051, 207052, 207053, 207054, 207055, 207056, 207057, 207058, 207059, 207060, 207061, 207062) c6( ) c7(R, wu, aW, jh, aT, dL, j, K, bK, g, wm, eL, JO); #c1(UBD) c2(NP_006389) c3(ubiquitin D) c4(164585, 164586, 164587) c5(164588, 164589, 164590, 164591, 164592, 164593, 164594, 164595, 164596, 164597, 164598, 164599, 164600, 164601, 164602, 164603, 164604, 164605, 164606, 164607, 164608, 164609, 164610, 164611, 164612, 164613, 164614, 164615, 164616, 164617, 164618, 164619) c6( ) c7(T, bO, bB, n, aj, ci, iI, V, q, J, hX, Gt, h, jj, rE, hH, af, bc, Gv, eZ, r, kp, hp, rR, bE, aUS, dy, KS, B, qc); #c1(UBE2A) c2(NP_001269090) c3(ubiquitin-conjugating enzyme EZ A isoform 4) c4( ) c5(100402, 100403, 100404, 100405, 100406, 100407) c6( ) c7(nf, atf, bxm, K, nA, WI, cSE); #c1(UBE2A) c2(NP_003327) c3(ubiquitin-conjugating enzyme E2 A isoform 1) c4(137499, 137500) c5(137501, 137502, 137503, 137504, 137505, 137506) c6( ) c7(nf, atf, bxm, K, nA, WI, cSE); #c1(UBE2A) c2(NP_861427) c3 (ubiquitin-conjugating enzyme E2 A isoform 2) c4(255557) c5(255558, 255559, 255560, 255561, 255562, 255563, 255564) c6( ) c7(nf, atf, bxm, K, nA, WI, cSE); #c1(UBE2B) c2(NP_003328) c3(ubiquitin-conjugating enzyme E2 B) c4(137507) c5(137508, 137509, 137510, 137511, 137512, 137513, 137514) c6( ) c7(T, gU, yk, if, jx, jh, ci, qN, eW, no, lg, aT, vH, h, K, ND, aW, hl, aq, awi, pa, bP, aSu, rH, B); #c1(UBE2C) c2(NP_001268671) c3(ubiquitin-conjugating enzyme E2 C isoform 7) c4( ) c5(100120, 100121, 100122, 100123, 100124, 100125) c6( ) c7(Fv, T, pf, if, jh, ci, qN, q, ad, eA, Vp, hd, aT, Fx, ae, d, B, s, qt, aW, ff, r, en, g, as, zE, cm, bz, Qy, bP, eM, nB, m, X); #c1(UBE2C) c2(NP_008950) c3(ubiquitin-conjugating enzyme E2 C isoform 1) c4(169717, 169718) c5(169719, 169720, 169721, 169722, 169723, 169724, 169725, 169726) c6( ) c7(Fv, T, pf, if, jh, ci, qN, q, ad, eA, Vp, hd, aT, Fx, ae, d, B, s, qt, aW, ff, r, en, g, as, zE, cm, bz, Qy, bP, eM, nB, m, X); #c1(UBE2C) c2(NP_861515) c3(ubiquitin-conjugating enzyme E2 C isoform 2) c4(255616) c5(255617, 255618, 255619, 255620, 255621, 255622) c6( ) c7(Fv, T, pf, if, jh, ci, qN, q, ad, eA, Vp, hd, aT, Fx, ae, d, B, s, qt, aW, ff, r, en, g, as, zE, cm, bz, Qy, bP, eM, nB, m, X); #c1(UBE2C) c2(NP_861516) c3(ubiquitin-conjugating enzyme E2 C isoform 3) c4(255623, 255624) c5(255625, 255626, 255627, 255628, 255629, 255630) c6( ) c7(Fv, T, pf, if, jh, ci, qN, q, ad, eA, Vp, hd, aT, Fx, ae, d, B, s, qt, aW, ff, r, en, g, as, zE, cm, bz, Qy, bP, eM, nB, m, X); #c1(UBE2C) c2(NP_861517) c3(ubiquitin-conjugating enzyme E2 C isoform 4) c4( ) c5(255631, 255632, 255633, 255634, 255635, 255636, 255637, 255638) c6( ) c7(Fv, T, pf, if, jh, ci, qN, q, ad, eA, Vp, hd, aT, Fx, ae, d, B, s, qt, aW, ff, r, en, g, as, zE, cm, bz, Qy, bP, eM, nB, m, X); #c1(UBEZD1) c2(NP_003329) c3(ubiquitin-conjugating enzyme E2 D1 isoform 1) c4(137515) c5(137516, 137517, 137518, 137519, 137520, 137521) c6( ) c7(T, yw, oW, yy, yu, af); #c1(UBE2D2) c2(NP_003330) c3 (ubiquitin-conjugating enzyme E2 D2 isoform 1) c4(137522, 137523) c5(137524, 137525, 137526, 137527, 137528, 137529, 137530, 137531, 137532, 137533, 137534, 137535) c6( ) c7(bw, T); #c1(UBE2D2) c2(NP_862821) c3(ubiquitin-conjugating enzyme E2 D2 isoform 2) c4( ) c5(255757, 255758, 255759, 255760, 255761, 255762, 255763, 255764) c6( ) c7(bw, T); #c1(UBEZD3) c2(NP_003331) c3(ubiquitin-conjugating enzyme E2 D3 isoform 1) c4(137536, 137537) c5(137538, 137539, 137540, 137541, 137542, 137543, 137544, 137545, 137546, 137547, 137548, 137549, 137550, 137551, 137552, 137553, 137554, 137555, 137556, 137557, 137558, 137559, 137560, 137561, 137562, 137563, 137564, 137565, 137566, 137567, 137568, 137569, 137570, 137571, 137572, 137573, 137574, 137575, 137576, 137577, 137578, 137579, 137580, 137581, 137582, 137583, 137584, 137585) c6( ) c7(aW, aj, ey, nB, aT); #c1(UBE2D3) c2(NP_871621) c3 (ubiquitin-conjugating enzyme E2 D3 isoform 2) c4(255924, 255925, 255926) c5(255927, 255928, 255929, 255930, 255931, 255932) c6( ) c7(aW, aj, ey, nB, aT); #c1(UBE2D3) c2(NP_871622) c3(ubiquitin-conjugating enzyme E2 D3 isoform 3) c4(255933) c5(255934, 255935, 255936, 255937, 255938, 255939) c6( ) c7(aW, aj, ey, nB, aT); #c1(UBEZE1) c2(NP_001189405) c3(ubiquitin-conjugating enzyme E2 E1 isoform 3) c4(79373) c5(79374, 79375, 79376, 79377, 79378, 79379) c6( ) c7(Pk, czP, lQ); #c1(UBEZE1) c2(NP_003332) c3(ubiquitin-conjugating enzyme E2 E1 isoform 1) c4(137586) c5(137587, 137588, 137589, 137590, 137591, 137592) c6( ) c7(Pk, czP, lQ); #c1(UBEZE1) c2(NP_872607) c3(ubiquitin-conjugating enzyme E2 E1 isoform 2) c4(256525) c5(256526, 256527, 256528, 256529, 256530, 256531) c6( ) c7(Pk, czP, lQ); #c1(UBEZE2) c2(NP_689866) c3(ubiquitin-conjugating enzyme E2 E2) c4(244202) c5(244203, 244204, 244205, 244206, 244207, 244208, 244209, 244210, 244211, 244212) c6( ) c7(bL, T, aW, oe, ht, ff, aT, en); #c1(UBEZE3) c2(NP_006348) c3(ubiquitin-conjugating enzyme E2 E3) c4(164222, 164223) c5(164224, 164225, 164226, 164227, 164228, 164229, 164230, 164231, 164232, 164233, 164234, 164235, 164236, 164237, 164238, 164239, 164240, 164241, 164242, 164243, 164244, 164245, 164246, 164247, 164248, 164249, 164250, 164251, 164252, 164253, 164254, 164255) c6( ) c7(aW, mR, hp, lg); #c1(UBEZG1) c2(NP_003333) c3(ubiquitin-conjugating enzyme E2 G1) c4(137593) c5(137594, 137595, 137596, 137597, 137598, 137599) c6( ) c7(T, ey); #c1(UBEZG2) c2(NP_003334) c3 (ubiquitin-conjugating enzyme E2 G2 isoform 1) c4(137600, 137601) c5(137602, 137603, 137604, 137605, 137606, 137607, 137608) c6( ) c7(ck); #c1(UBE2G2) c2(NP_872630) c3(ubiquitin-conjugating enzyme E2 G2 isoform 2) c4( ) c5(256572, 256573, 256574, 256575, 256576) c6( ) c7(ck); #c1(UBE2H) c2(NP_003335) c3(ubiquitin-conjugating enzyme E2 H isoform 1) c4(137609) c5(137610, 137611, 137612, 137613, 137614, 137615) c6( ) c7(kl, bN); #c1(UBE2H) c2(NP_874356) c3(ubiquitin-conjugating enzyme E2 H isoform 2) c4(256598) c5(256599, 256600, 256601, 256602, 256603, 256604) c6( ) c7(kl, bN); #c1(UBE2I) c2(NP_003336) c3(SUMO-conjugating enzyme UBC9) c4(137616) c5(137617, 137618, 137619, 137620, 137621, 137622, 137623, 137624, 137625, 137626, 137627, 137628, 137629, 137630, 137631, 137632, 137633, 137634, 137635, 137636, 137637, 137638, 137639, 137640, 137641, 137642) c6( ) c7(T, sw, aA, fd, v, ci, hF, zP, ip, iu, arA, aT, K, af, z, aW, Y, wc, aF, g, t, bw, bz, ck, Di, eM, bt, iS, m, vt, B); #c1(UBEZJ1) c2(NP_057105) c3 (ubiquitin-conjugating enzyme E2 J1) c4(188917, 188918) c5(188919, 188920, 188921, 188922, 188923, 188924, 188925, 188926, 188927, 188928, 188929, 188930) c6( ) c7(jf); #c1(UBE2K) c2(NP_001104582) c3(ubiquitin-conjugating enzyme E2 K isoform 2) c4(42193) c5(42194, 42195, 42196, 42197, 42198, 42199) c6( ) c7(T, rR, bgk, B, aiB, if, alg, ht, cSF, bUn, aT, agl, jh, qN, hu, rP, cnS); #c1(UBE2K) c2(NP_001104583) c3(ubiquitin-conjugating enzyme E2 K isoform 3) c4(42200) c5(42201, 42202, 42203, 42204, 42205, 42206) c6( ) c7(T, rR, bgk, B, aiB, if, alg, ht, cSF, bUn, aT, agl, jh, qN, hu, rP, cnS); #c1(UBE2K) c2(NP_005330) c3(ubiquitin-conjugating enzyme E2 K isoform 1) c4(155043) c5(155044, 155045, 155046, 155047, 155048, 155049) c6( ) c7(T, rR, bgk, B, aiB, if, alg, ht, cSF, bUn, aT, agl, jh, qN, hu, rP, cnS); #c1(UBE2L3) c2(NP_001243284) c3 (ubiquitin-conjugating enzyme E2 L3 isoform 4) c4(90368) c5(90369, 90370, 90371, 90372, 90373, 90374) c6( ) c7(eZ, ey, hp, jE, iV, y, ae, ad, hX); #c1(UBE2L3) c2(NP_001243285) c3(ubiquitin-conjugating enzyme E2 L3 isoform 3) c4(90375) c5(90376, 90377, 90378, 90379, 90380, 90381) c6( ) c7(eZ, ey, hp, jE, iV, y, ae, ad, hX); #c1(UBE2L3) c2(NP_003338) c3(ubiquitin-conjugating enzyme E2 L3 isoform 1) c4(137643) c5(137644, 137645, 137646, 137647, 137648, 137649) c6( ) c7(eZ, ey, hp, jE, iV, y, ae, ad, hX); #c1(UBE2L6) c2(NP_004214) c3(ubiquitin/ISG15-conjugating enzyme E2 L6 isoform 1) c4(144335, 144336, 144337) c5(144338, 144339, 144340, 144341, 144342, 144343, 144344, 144345) c6( ) c7(aW, ds, T, aT); #c1(UBE2L6) c2(NP_937826) c3(ubiquitin/ISG15-conjugating enzyme E2 L6 isoform 2) c4( ) c5(259225, 259226, 259227, 259228, 259229, 259230, 259231, 259232, 259233, 259234) c6( ) c7(aW, ds, T, aT); #c1(UBE2M) c2(NP_003960) c3(NEDD8-conjugating enzyme Ubcl2) c4(142472) c5(142473, 142474, 142475, 142476, 142477, 142478) c6( ) c7(T, id); #c1(UBE2N) c2(NP_003339) c3(ubiquitin-conjugating enzyme E2 N) c4(137650) c5(137651, 137652, 137653, 137654, 137655, 137656) c6( ) c7(T, if, BF, eA, br, av, b, jz, JT, io, dL, K, eL, asp, ff, cr, lo, agF, en, g, as, t, cl, bz, cm, bw, eM, hL, m, i, it, B); #c1(UBE2N) c2(XP_011537021) c3(ubiquitin-conjugating enzyme E2 N isoform X1) c4( ) c5(334779) c6( ) c7(T, if, BF, eA, br, av, b, jz, JT, io, dL, K, eL, asp, ff, cr, lo, agF, en, g, as, t, cl, bz, cm, bw, eM, hL, m, i, it, B); #c1(UBE2Q2) c2(NP_001138807) c3(ubiquitin-conjugating enzyme E2 Q2 isoform 2) c4(57764) c5(57765, 57766, 57767, 57768, 57769, 57770, 57771, 57772, 57773, 57774) c6( ) c7(T, eR, aW, aT, aBx, iK); #c1(UBE2Q2) c2(NP_775740) c3(ubiquitin-conjugating enzyme E2 Q2 isoform 1) c4(250140) c5(250141, 250142, 250143, 250144, 250145, 250146) c6( ) c7(T, eR, aW, aT, aBx, iK); #c1(UBE2Q2) c2(XP_005254844) c3 (ubiquitin-conjugating enzyme E2 Q2 isoform X2) c4(273336) c5(273337, 273338, 273339, 273340, 273341, 273342) c6( ) c7(T, eR, aW, aT, aBx, iK); #c1(UBE2AL1) c2(NP_001138633) c3(ubiquitin-conjugating enzyme E20-like protein 1) c4( ) c5(57303, 57304, 57305, 57306, 57307) c6( ) c7(hH, fr, es); #c1(UBE2AL1) c2(XP_005248304) c3(ubiquitin-conjugating enzyme E2Q-like protein 1 isoform X1) c4(268300) c5(268301) c6( ) c7(hH, fr, es); #c1(UBE2R2) c2(NP_060281) c3(ubiquitin-conjugating enzyme E2 R2) c4(195181) c5(195182, 195183, 195184, 195185, 195186, 195187) c6( ) c7(ac); #c1(UBE2S) c2(NP_055316) c3(ubiquitin-conjugating enzyme E2 S) c4(180402) c5(180403, 180404, 180405, 180406, 180407, 180408) c6( ) c7(T, bgk, B, aiB, aW, if, alg, ht, cSF, bUn, aT, agl, jh, qN, hu, rP, cnS); #c1(UBE2T) c2(NP_054895) c3(ubiquitin-conjugating enzyme E2 T) c4(178241) c5(178242, 178243, 178244, 178245, 178246, 178247) c6( ) c7(av, bmc, os, T, dD, rR, qt, aW, Y, dE, ea, aT, nf, oZ, eq, C, iK, B); #c1(UBE2U) c2(XP_011539067) c3(ubiquitin-conjugating enzyme E2 U isoform X1) c4(337139, 337140) c5(337141, 337142, 337143, 337144, 337145, 337146) c6( ) c7(fq); #c1(UBE2V1) c2(NP_001027459) c3 (ubiquitin-conjugating enzyme E2 variant 1 isoform d) c4(30215) c5(30216, 30217, 30218, 30219, 30220, 30221) c6( ) c7(T, dA, WT, ci, PJ, eW, ad, hX, h, BZ, K, ae, avQ, ak, anR, d, CT, lo, as, pb, ck, Qi, iO, vW, c, iT, eB, U, bC, bt, xg, B); #c1(UBE2V1) c2(NP_001244322) c3 (ubiquitin-conjugating enzyme E2 variant 1 isoform a) c4(92163) c5(92164, 92165, 92166, 92167, 92168, 92169, 92170, 92171, 92172, 92173) c6( ) c7(T, dA, WT, ci, PJ, eW, ad, hX, h, BZ, K, ae, avQ, ak, anR, d, CT, lo, as, pb, ck, Qi, iO, vW, c, iT, eB, U, bC, bt, xg, B); #c1(UBE2V1) c2(NP_001244325) c3(ubiquitin-conjugating enzyme E2 variant 1 isoform f) c4(92174) c5(92175, 92176, 92177, 92178, 92179, 92180) c6( ) c7(T, dA, WT, ci, PJ, eW, ad, hX, h, BZ, K, ae, avQ, ak, anR, d, CT, lo, as, pb, ck, Qi, iO, vW, c, iT, eB, U, bC, bt, xg, B); #c1(UBE2V1) c2(NP_071887) c3(ubiquitin-conjugating enzyme E2 variant 1 isoform c) c4(212084, 212085) c5(212086, 212087, 212088, 212089, 212090, 212091, 212092, 212093, 212094, 212095, 212096, 212097, 212098, 212099, 212100, 212101) c6( ) c7(T, dA, WT, ci, PJ, eW, ad, hX, h, BZ, K, ae, avQ, ak, anR, d, CT, lo, as, pb, ck, Di, iO, vW, c, iT, eB, U, bC, bt, xg, B); #c1(UBE2V2) c2(NP_003341) c3(ubiquitin-conjugating enzyme E2 variant 2) c4(137657) c5(137658, 137659, 137660, 137661, 137662, 137663) c6( ) c7(K); #c1(UBE2Z) c2(NP_075567) c3 (ubiquitin-conjugating enzyme E2 Z) c4(213972, 213973) c5(213974, 213975, 213976, 213977, 213978, 213979, 213980, 213981, 213982, 213983, 213984, 213985) c6( ) c7(T, acu, gC, cr, j, gu, m, t, hq); #c1(UBE3A) c2(XP_005268328) c3(ubiquitin-protein ligase E3A isoform X1) c4(281739) c5(281740, 281741, 281742, 281743, 281744, 281745, 281746, 281747) c6( ) c7(T, FR, if, ha, n, nA, qN, rc, aR, ad, boY, lg, aT, K, aW, Az, aPP, XI, dr, bN, Di, rR, cSG, iT, agl, U, bt); #c1(UBE3A) c2(XP_006720737) c3(ubiquitin-protein ligase E3A isoform X2) c4(293644, 293645) c5(293646, 293647, 293648, 293649, 293650, 293651, 293652, 293653, 293654, 293655, 293656, 293657, 293658, 293659) c6( ) c7(T, fR, if, ha, n, nA, qN, rc, aR, ad, boY, lg, aT, K, aW, Az, aPP, XI, dr, bN, Qi, rR, cSG, iT, agl, U, bt); #c1(UBE3A) c2(XP_011520297) c3(ubiquitin-protein ligase E3A isoform X3) c4(315143) c5(315144, 315145, 315146, 315147, 315148, 315149) c6( ) c7(T, fR, if, ha, n, nA, qN, rc, aR, ad, boY, lg, aT, K, aW, Az, aPP, XI, dr, bN, Qi, rR, cSG, iT, agl, U, bt); #c1(UBE3B) c2(NP_001257380) c3(ubiquitin-protein ligase E3B isoform 3) c4(95275) c5(95276, 95277, 95278, 95279, 95280, 95281, 95282, 95283, 95284, 95285, 95286, 95287, 95288, 95289) c6( ) c7(cSF, amj, cBg, WI, cm, cSH); #c1(UBE3B) c2(NP_569733) c3(ubiquitin-protein ligase E3B isoform 1) c4(233883, 233884) c5(233885, 233886, 233887, 233888, 233889, 233890, 233891, 233892, 233893, 233894, 233895, 233896, 233897, 233898, 233899, 233900, 233901, 233902, 233903, 233904, 233905, 233906) c6( ) c7(cSF, amj, cBg, WI, cm, cSH); #c1(UBE3B) c2(XP_006719745) c3(ubiquitin-protein ligase E3B isoform X4) c4(292958) c5(292959, 292960, 292961, 292962, 292963, 292964) c6( ) c7(cSF, amj, cBg, WI, cm, cSH); #c1(UBE3B) c2(XP_011537262) c3(ubiquitin-protein ligase E3B isoform X2) c4(334989) c5(334990, 334991, 334992, 334993, 334994, 334995) c6( ) c7(cSF, amj, cBg, WI, cm, cSH); #c1(UBE3B) c2(XP_011537263) c3(ubiquitin-protein ligase E3B isoform X5) c4( ) c5(334996) c6( ) c7(cSF, amj, cBg, WI, cm, cSH); #c1(UBE3B) c2(XP_011537264) c3(ubiquitin-protein ligase E3B isoform X6) c4(334997) c5( ) c6( ) c7(cSF, amj, cBg, WI, cm, cSH); #c1(UBE3C) c2(NP_055486) c3(ubiquitin-protein ligase E3C) c4(181434) c5(181435, 181436, 181437, 181438, 181439, 181440) c6( ) c7(aDy, bM, B); #c1(UBE3C) c2(XP_005249621) c3(ubiquitin-protein ligase E3C isoform X2) c4(269757) c5(269758, 269759, 269760, 269761, 269762, 269763) c6( ) c7(aDy, bM, B); #c1(UBE4A) c2(NP_001191006) c3 (ubiquitin conjugation factor E4 A isoform 2) c4(80511) c5(80512, 80513, 80514, 80515, 80516, 80517) c6( ) c7(T, lR, K, iV, cm, pb); #c1(UBE4A) c2(NP_004779) c3(ubiquitin conjugation factor E4 A isoform 1) c4(149554, 149555) c5(149556, 149557, 149558, 149559, 149560, 149561, 149562, 149563, 149564, 149565, 149566, 149567) c6( ) c7(T, lR, K, iV, cm, pb); #c1(UBE4B) c2(XP_011538792) c3(ubiquitin conjugation factor E4 B isoform X4) c4( ) c5(336537, 336538, 336539, 336540, 336541, 336542) c6( ) c7(T, cc, aq, lR, K, jE, pb, cm, B); #c1(UBIAD1) c2(XP_011539606) c3(ubiA prenyltransferase domain-containing protein 1 isoform X2) c4( ) c5(337843, 337844, 337845, 337846, 337847, 337848) c6( ) c7(aDu, py, cH, qb, eL, dL, fr, oQ, cSl, ae, cd, ad); #c1(UBL3) c2(NP_009037) c3(ubiquitin-like protein 3 precursor) c4(170356) c5(170357, 170358, 170359, 170360, 170361, 170362) c6( ) c7(yy, Fq); #c1(UBL4A) c2(NP_055050) c3(ubiquitin-like protein 4A) c4(178602) c5(178603, 178604, 178605, 178606, 178607, 178608) c6( ) c7(agx, T, qo, byy, qb, KW, fd, KO, mN, qf, nA, agz, eg, aCi, b, vJ, oj, aT, oW, dV, dz, UD, bK, K, h, yj, byw, iU, aW, hC, xs, uY, S, Ku, avG, Pd, hJ, BG, zz, dr, adA, byB, BU, VJ, ac, qe, yN, byG, cj, hO, mf, pw, ER, CI, i, lC, X); #c1(UBL5) c2(NP_001041706) c3(ubiquitin-like protein 5) c4(34309) c5(34310, 34311, 34312, 34313, 34314, 34315, 34316, 34317, 34318, 34319) c6( ) c7(T, qn, bcb, fd, aj, v, jj, bgR, J, hX, aE, qh, aT, cU, kH, h, iz, je, aW, hp, iM, eM, aiM, wR, ei, bt, ao); #c1(UBL7) c2(NP_001273669) c3(ubiquitin-like protein 7 isoform a) c4( ) c5(105584, 105585, 105586, 105587, 105588, 105589, 105590, 105591, 105592) c6( ) c7(Cw); #c1(UBL7) c2(NP_001273671) c3(ubiquitin-like protein 7 isoform b) c4(105593) c5(105594) c6( ) c7(Cw); #c1(UBLCP1) c2(NP_659486) c3(ubiquitin-like domain-containing CTD phosphatase 1) c4(240172) c5(240173, 240174, 240175, 240176, 240177, 240178) c6( ) c7(vr, cr); #c1(UBN1) c2(NP_001072982) c3(ubinuclein-1 isoform a) c4(35424) c5(35425, 35426, 35427, 35428, 35429, 35430, 35431, 35432, 35433, 35434) c6( ) c7(bxS, c); #c1(UBN1) c2(NP_001275585) c3 (ubinuclein-1 isoform b) c4(106849) c5(106850, 106851, 106852, 106853, 106854, 106855) c6( ) c7(bxS, c); #c1(UBOX5) c2(NP_001254513) c3(RING finger protein 37 isoform c) c4(94590) c5( ) c6( ) c7(LB); #c1(UBOX5) c2(NP_055763) c3(RING finger protein 37 isoform a) c4(183336) c5(183337, 183338, 183339, 183340, 183341, 183342) c6( ) c7(LB); #c1(UBOX5) c2(NP_955447) c3(RING finger protein 37 isoform b) c4(261724) c5(261725, 261726, 261727, 261728, 261729, 261730, 261731, 261732, 261733, 261734, 261735, 261736) c6( ) c7(LB); #c1(UBP1) c2(NP_001121632) c3(upstream-binding protein 1 isoform LBP-la) c4(45913) c5(45914, 45915, 45916, 45917, 45918, 45919, 45920, 45921, 45922, 45923, 45924, 45925) c6( ) c7(h, hH); #c1(UBP1) c2(NP_001121633) c3(upstream-binding protein 1 isoform LBP-(b) c4(45926, 45927) c5(45928, 45929, 45930, 45931, 45932, 45933, 45934, 45935, 45936, 45937, 45938) c6( ) c7(h, hH); #c1(UBQLN1) c2(NP_038466) c3(ubiquilin-1 isoform 1) c4(176563) c5(176564, 176565, 176566, 176567, 176568, 176569) c6( ) c7(av, iu, rR, c, aW, ex, cz, aT, eV, K, cGn, an, as, cm, af); #c1(UBOLN1) c2(NP_444295) c3(ubiquilin-1 isoform 2) c4(230316) c5(230317, 230318, 230319, 230320, 230321, 230322) c6( ) c7(av, iu, rR, c, aW, ex, cz, aT, eV, K, cGn, an, as, cm, af); #c1(UBQLN2) c2(NP_038472) c3(ubiquilin-2) c4(176599, 176600) c5(176601, 176602, 176603, 176604, 176605, 176606, 176607, 176608, 176609, 176610, 176611, 176612) c6( ) c7(bb, c, axR, WY, cSJ, aaE, PJ); #c1(UBQLNL) c2(NP_659490) c3(ubiquilin-like protein) c4(240186, 240187) c5(240188, 240189, 240190, 240191, 240192, 240193, 240194, 240195, 240196, 240197, 240198, 240199) c6( ) c7(T); #c1(UBR1) c2(NP_777576) c3(E3 ubiquitin-protein ligase UBR1) c4(251202, 251203, 251204) c5(251205, 251206, 251207, 251208, 251209, 251210, 251211, 251212, 251213, 251214, 251215, 251216, 251217, 251218, 251219, 251220, 251221, 251222) c6( ) c7(ar, T, fM, aKG, cSK, nA); #c1(UBR3) c2(NP_742067) c3(E3 ubiquitin-protein ligase UBR3) c4(247939, 247940) c5(247941, 247942, 247943, 247944, 247945, 247946, 247947, 247948, 247949, 247950, 247951, 247952, 247953, 247954, 247955, 247956) c6( ) c7(eu, VZ, ac, M); #c1(UBR3) c2(XP_005246357) c3(E3 ubiquitin-protein ligase UBR3 isoform X3) c4(267082) c5(267083, 267084, 267085, 267086, 267087, 267088) c6( ) c7(eu, VZ, ac, M); #c1(UBR4) c2(XP_011539410) c3(E3 ubiquitin-protein ligase UBR4 isoform X1) c4( ) c5(337528, 337529, 337530, 337531, 337532, 337533, 337534, 337535, 337536, 337537, 337538, 337539, 337540) c6( ) c7(T, Y, aA, aF, aan, akr); #c1(UBR4) c2(XP_011539414) c3(E3 ubiquitin-protein ligase UBR4 isoform X5) c4(337541) c5(337542, 337543, 337544, 337545, 337546, 337547) c6( ) c7(T, Y, aA, aF, aan, akr); #c1(UBR4) c2(XP_011539419) c3(E3 ubiquitin-protein ligase UBR4 isoform X10) c4(337548) c5( ) c6( ) c7(T, Y, aA, aF, aan, akr); #c1(UBR4) c2(XP_011539420) c3(E3 ubiquitin-protein ligase UBR4 isoform X11) c4(337549, 337550) c5( ) c6( ) c7(T, Y, aA, aF, aan, akr); #c1(UBR4) c2(XP_011539421) c3(E3 ubiquitin-protein ligase UBR4 isoform X12) c4(337551) c5(337552, 337553, 337554, 337555, 337556, 337557) c6( ) c7(T, Y, aA, aF, aan, akr); #c1(UBR4) c2(XP_011539422) c3(E3 ubiquitin-protein ligase UBR4 isoform X13) c4(337558, 337559) c5(337560, 337561, 337562, 337563, 337564, 337565, 337566, 337567, 337568, 337569, 337570, 337571) c6( ) c7(T, Y, aA, aF, aan, akr); #c1(UBR4) c2(XP_011539423) c3(E3 ubiquitin-protein ligase UBR4 isoform X14) c4(337572, 337573) c5(337574, 337575, 337576, 337577, 337578, 337579, 337580, 337581, 337582, 337583, 337584, 337585) c6( ) c7(T, Y, aA, af, aan, akr); #c1(UBR5) c2(NP_001269802) c3(E3 ubiquitin-protein ligase UBR5 isoform 2) c4(102865, 102866) c5(102867, 102868, 102869, 102870, 102871, 102872, 102873, 102874, 102875, 102876, 102877, 102878) c6( ) c7(bF, T, bgk, B, aiB, aW, if, agl, cSF, bUn, aT, aA, jh, aF, m, ci, qN, t, cnS); #c1(UBR5) c2(NP_056986) c3(E3 ubiquitin-protein ligase UBR5 isoform 1) c4(188341) c5(188342, 188343, 188344, 188345, 188346, 188347) c6( ) c7(bF, T, bgk, B, aiB, aW, if, agl, cSF, bUn, aT, aA, jh, aF, m, ci, qN, t, cnS); #c1(UBR5) c2(XP_005251019) c3(E3 ubiquitin-protein ligase UBR5 isoform X4) c4( ) c5(270723, 270724, 270725, 270726, 270727, 270728) c6( ) c7(bF, T, bgk, B, aiB, aW, if, agl, cSF, bUn, aT, aA, jh, aF, m, ci, qN, t, cnS); #c1(UBR5) c2(XP_011515407) c3(E3 ubiquitin-protein ligase UBR5 isoform X2) c4(308235) c5( ) c6( ) c7(bF, T, bgk, B, aiB, aW, if, agl, cSF, bUn, aT, aA, jh, aF, m, ci, qN, t, cnS); #c1(UBR7) c2(NP_786924) c3(putative E3 ubiquitin-protein ligase UBR7) c4(251916, 251917, 251918) c5(251919, 251920, 251921, 251922, 251923, 251924, 251925, 251926, 251927, 251928, 251929, 251930, 251931, 251932, 251933, 251934, 251935, 251936, 251937, 251938) c6( ) c7(nA); #c1(UBTD2) c2(NP_689490) c3(ubiquitin domain-containing protein 2) c4(242877) c5(242878, 242879, 242880, 242881, 242882, 242883) c6( ) c7(j); #c1(UBTF) c2(NP_001070151) c3(nucleolar transcription factor 1 isoform b) c4(34611) c5(34612, 34613, 34614, 34615, 34616, 34617, 34618, 34619, 34620, 34621, 34622, 34623, 34624, 34625, 34626, 34627, 34628, 34629) c6( ) c7(aq, y, B, K); #c1(UBTF) c2(XP_006722123) c3(nucleolar transcription factor 1 isoform X1) c4(294611) c5(294612, 294613, 294614, 294615, 294616, 294617, 294618, 294619, 294620, 294621, 294622, 294623, 294624, 294625, 294626) c6( ) c7(aq, y, B, K); #c1(UBXN1) c2(NP_001273006) c3(UBX domain-containing protein 1 isoform 2) c4(104156, 104157, 104158) c5(104159, 104160, 104161, 104162, 104163, 104164, 104165, 104166, 104167, 104168, 104169, 104/70, 104/71) c6( ) c7(aA); #c1(UBXN1) c2(NP_001273007) c3(UBX domain-containing protein 1 isoform 3) c4( ) c5(104/72, 104173, 104174, 104175, 104176, 104/77) c6( ) c7(aA); #c1(UBXN1) c2(NP_056937) c3(UBX domain-containing protein 1 isoform 1) c4(188153) c5(188154, 188155, 188156, 188157, 188158, 188159) c6( ) c7(aA); #c1(UBXN2A) c2(NP_859064) c3(UBX domain-containing protein 2A) c4(255456, 255457) c5(255458, 255459, 255460, 255461, 255462, 255463, 255464, 255465, 255466, 255467, 255468, 255469, 255470, 255471, 255472, 255473) c6( ) c7(bB, bE); #c1(UBXN2A) c2(XP_011530936) c3(UBX domain-containing protein 2A isoform X2) c4(326916) c5(326917, 326918, 326919, 326920) c6(326921, 326922) c7(bB, bE); #c1(UBXN2B) c2(NP_001071087) c3(UBX domain-containing protein 2B) c4(35267) c5(35268, 35269, 35270, 35271, 35272, 35273) c6( ) c7(Pg); #c1(UBXN4) c2(NP_055422) c3(UBX domain-containing protein 4) c4(181063, 181064) c5(181065, 181066, 181067, 181068, 181069, 181070, 181071, 181072, 181073, 181074, 181075, 181076) c6( ) c7(ab, af, K); #c1(UCHL1) c2(NP_004172) c3 (ubiquitin carboxyl-terminal hydrolase isozyme LI) c4(144059, 144060, 144061) c5(144062, 144063, 144064, 144065, 144066, 144067, 144068, 144069, 144070, 144071, 144072, 144073, 144074, 144075, 144076, 144077, 144078, 144079) c6( ) c7(bL, T, fM, apr, if, iW, ht, fd, jh, aA, GC, n, aj, dG, v, ci, bX, qN, fr, AO, q, aR, J, ad, bv, av, l, cSL, fX, u, nv, gd, aT, cU, h, K, hB, an, NM, sX, dx, cSM, ae, af, ak, lR, s, xR, aW, Y, ff, r, awu, EH, aF, bSc, en, g, t, cm, bz, iK, GB, Wt, rR, YE, aDA, eM, bP, wk, gX, U, Lq, m, rH, Xu, zu, it, B); #c1(UCHL3) c2(NP_005993) c3(ubiquitin carboxyl-terminal hydrolase isozyme L3 isoform 2) c4(161077) c5(161078, 161079, 161080, 161081, 161082, 161083) c6( ) c7(ak, if, g, U); #c1(UCHL3) c2(XP_011533514) c3(ubiquitin carboxyl-terminal hydrolase isozyme L3 isoform X1) c4( ) c5(329607, 329608) c6( ) c7(ak, if, g, U); #c1(UCHL3) c2(XP_011533516) c3 (ubiquitin carboxyl-terminal hydrolase isozyme L3 isoform X3) c4(329609, 329610) c5(329611, 329612, 329613, 329614, 329615, 329616, 329617, 329618, 329619, 329620, 329621) c6( ) c7(ak, if, g, U); #c1(UCHL5) c2(NP_001186190) c3(ubiquitin carboxyl-terminal hydrolase isozyme L5 isoform 2) c4(77328) c5( ) c6( ) c7(T, if, fd, n, bq, jb, it); #c1(UCHL5) c2(NP_00186191) c3(ubiquitin carboxyl-terminal hydrolase isozyme L5 isoform 3) c4(77329) c5(77330, 77331, 77332, 77333, 77334, 77335, 77336, 77337, 77338, 77339, 77340, 77341, 77342, 77343, 77344, 77345, 77346, 77347, 77348, 77349, 77350, 77351, 77352, 77353, 77354, 77355, 77356, 77357, 77358, 77359) c6( ) c7(T, if, fd, n, bq, jb, it); #c1(UCHL5) c2(NP_001186192) c3(ubiquitin carboxyl-terminal hydrolase isozyme [5 isoform 4) c4(77360) c5(77361, 77362, 77363, 77364, 77365, 77366, 77367, 77368, 77369, 77370, 77371, 77372, 77373, 77374, 77375) c6( ) c7(T, if, fd, n, bq, jb, it); #c1(UCHL5) c2(NP_057068) c3(ubiquitin carboxyl-terminal hydrolase isozyme L5 isoform 1) c4(188755) c5(188756, 188757, 188758, 188759, 188760) c6( ) c7(T, if, fd, n, bq, jb, it); #c1(UCHL5) c2(XP_005245301) c3(ubiquitin carboxyl-terminal hydrolase isozyme L5 isoform X2) c4(266416) c5( ) c6( ) c7(T, if, fd, n, bq, jb, it); #c1(UCHL5) c2(XP_005245302) c3(ubiquitin carboxyl-terminal hydrolase isozyme L5 isoform X8) c4( ) c5(266417) c6( ) c7(T, if, fd, n, bq, jb, it); #c1(UCHL5) c2(XP_005245303) c3(ubiquitin carboxyl-terminal hydrolase isozyme L5 isoform X9) c4(266418, 266419, 266420, 266421, 266422) c5(266423, 266424, 266425, 266426, 266427) c6( ) c7(T, if, fd, n, bq, jb, it); #c1(UCHL5) c2(XP_005245305) c3(ubiquitin carboxyl-terminal hydrolase isozyme L5 isoform X13) c4(266428) c5(266429, 266430, 266431, 266432, 266433, 266434) c6( ) c7(T, if, fd, n, bq, jb, it); #c1(UCHL5) c2(XP_005245306) c3(ubiquitin carboxyl-terminal hydrolase isozyme L5 isoform X16) c4(266435) c5(266436, 266437, 266438, 266439, 266440) c6( ) c7(T, if, fd, n, bq, jb, it); #c1(UCHL5) c2(XP_006711429) c3 (ubiquitin carboxyl-terminal hydrolase isozyme L5 isoform X1) c4(286010) c5( ) c6( ) c7(T, if, fd, n, bq, jb, it); #c1(UCHL5) c2(XP_006711433) c3(ubiquitin carboxyl-terminal hydrolase isozyme L5 isoform X10) c4(286011) c5(286012, 286013, 286014, 286015, 286016, 286017) c6( ) c7(T, if, fd, n, bq, jb, it); #c1(UCHL5) c2(XP_011507907) c3(ubiquitin carboxyl-terminal hydrolase isozyme L5 isoform X5) c4( ) c5(298207, 298208, 298209, 298210, 298211, 298212) c6( ) c7(T, if, fd, n, bq, jb, it); #c1(UCHL5) c2(XP_011507908) c3(ubiquitin carboxyl-terminal hydrolase isozyme L5 isoform X7) c4(298213) c5(298214, 298215, 298216, 298217, 298218, 298219, 298220, 298221, 298222, 298223, 298224) c6( ) c7(T, if, fd, n, bq, jb, it); #c1(UCK1) c2(NP_001129426) c3(uridine-cytidine kinase 1 isoform b) c4(51476) c5(51477, 51478, 51479, 51480, 51481, 51482) c6( ) c7(bw, cbm, np); #c1(UCK1) c2(NP_001248379) c3(uridine-cytidine kinase 1 isoform c) c4(93905) c5(93906, 93907, 93908, 93909, 93910, 93911) c6( ) c7(bw, cbm, np); #c1(UCK1) c2(NP_001248380) c3(uridine-cytidine kinase 1 isoform d) c4(93912) c5(93913, 93914, 93915, 93916, 93917, 93918) c6( ) c7(bw, cbm, np); #c1(UCK1) c2(NP_113620) c3(uridine-cytidine kinase 1 isoform a) c4(221767) c5(221768, 221769, 221770, 221771, 221772, 221773) c6( ) c7(bw, cbm, np); #c1(UCK2) c2(NP_036606) c3(uridine-cytidine kinase 2) c4(174780, 174781) c5(174782, 174783, 174784, 174785, 174786, 174787, 174788, 174789, 174790, 174791, 174792, 174793) c6( ) c7(ac, ds, bu, cz, r, jh, Ld, bq, q, bA); #c1(UCKL1) c2(NP_001180308) c3(uridine-cytidine kinase-like 1 isoform 2) c4(73808, 73809) c5(73810, 73811, 73812, 73813, 73814, 73815, 73816, 73817, 73818, 73819, 73820, 73821) c6( ) c7(T); #c1(UCKL1) c2(NP_060329) c3 (uridine-cytidine kinase-like 1 isoform 1) c4(195413, 195414, 195415) c5(195416, 195417, 195418, 195419, 195420, 195421, 195422, 195423, 195424, 195425, 195426, 195427) c6( ) c7(T); #c1(UCKL1) c2(XP_005260273) c3 (uridine-cytidine kinase-like 1 isoform X4) c4(276507) c5( ) c6( ) c7(T); #c1(UCKL1) c2(XP_006723872) c3(uridine-cytidine kinase-like 1 isoform X5) c4( ) c5(295416) c6( ) c7(T); #c1(UCKL1) c2(XP_011527175) c3(uridine-cytidine kinase-like 1 isoform X11) c4( ) c5(321679, 321680, 321681, 321682, 321683) c6( ) c7(T); #c1(UCKL1) c2(XP_011527176) c3 (uridine-cytidine kinase-like 1 isoform X12) c4( ) c5(321684, 321685, 321686, 321687, 321688, 321689) c6( ) c7(T); #c1(UCMA) c2(NP_660357) c3(unique cartilage matrix-associated protein isoform 1 preproprotein) c4(241078) c5(241079, 241080, 241081, 241082, 241083, 241084, 241085, 241086) c6(241087, 241088) c7(T, aA, fr, eA, ad, av, ft, zw, aE, pX, aT, K, ae, gj, aW, aF, g, cm, t, bz, iK, es, eM, iA, m, lS, y); #c1(UCN) c2(NP_003344) c3(urocortin preproprotein) c4(137664) c5(137665, 137666, 137667, 137668, 137669, 137670) c6( ) c7(bM, T, os, aD, em, e, bO, cz, eb, fd, aj, bS, j, ct, nA, v, aNG, oZ, Fb, fr, eA, ad, eW, dP, x, py, aEX, hb, Bc, aT, LA, cy, hn, ji, K, he, ae, yo, s, aW, Y, aoo, az, ff, aq, jh, en, cl, Z, t, hq, axl, kP, ac, eM, bE, AM, iT, hl, cj, JM, acJ, k, m, y, bt, Bh, B); #c1(UCN2) c2(NP_149976) c3(urocortin-2 preproprotein) c4(227714) c5(227715, 227716, 227717, 227718, 227719, 227720) c6( ) c7(dP, Bv, r, ji, q, y, ZW, cd, cDV); #c1(UCN3) c2(NP_444277) c3(urocortin-3 preproprotein) c4(230241) c5(230242, 230243, 230244, 230245, 230246, 230247, 230248, 230249) c6(230250, 230251) c7(T, bO, cz, n, v, Gn, ci, bd, eW, aMB, dP, av, K, aa, s, al, Hu, g, Z, bz, hq, eM, y, iu); #c1(UCP1) c2(NP_068605) c3(mitochondrial brown fat uncoupling protein 1) c4(209782) c5(209783, 209784, 209785, 209786, 209787, 209788) c6( ) c7(bL, T, dD, eR, ea, oe, eX, jh, cz, bB, aj, v, j, eq, bX, cd, J, ad, uT, aT, eV, K, qM, de, ae, A, aoX, cH, cW, aW, hl, Mz, dq, bJ, bE, Fi, vy, hj, ei, bTg); #c1(UCP2) c2(NP_003346) c3(mitochondrial uncoupling protein 2) c4(137671, 137672) c5(137673, 137674, 137675, 137676, 137677, 137678, 137679, 137680, 137681, 137682, 137683, 137684) c6( ) c7(T, ea, e, bB, aoj, ci, aY, Bc, iX, M, K, qM, zl, bbC, aW, fc, hN, vs, nB, lp, wE, U, vy, Hb, dD, aw, aj, zP, cd, bSq, do, jL, jy, ey, uV, vx, A, fq, bJ, bo, hl, qc, bL, fM, eR, qQ, cz, sM, cR, hz, v, j, C, V, GS, J, Yn, uT, aT, cy, bq, eu, kl, Y, kC, ck, pO, c, ac, hr, mf, cSN, cS, gT, aZ, ha, dG, eq, ad, fU, hD, uX, cr, eZ, dq, iV, bBu, bz, bE, ei, y, X); #c1(UCP3) c2(NP_003347) c3(mitochondrial uncoupling protein 3 isoform UCP3L) c4(137685, 137686, 137687) c5(137688, 137689, 137690, 137691, 137692, 137693) c6( ) c7(bL, dD, eR, e, aw, qQ, cz, aj, dG, j, v, eq, cd, J, ad, uT, Bc, iX, jy, dL, K, qM, bq, af, A, Mz, dq, iy, g, cM, aHG, bJ, D, c, hr, at, mf, ei, gT); #c1(UCP3) c2(NP_073714) c3(mitochondrial uncoupling protein 3 isoform UCP3S) c4(213200) c5(213201, 213202, 213203, 213204, 213205, 213206, 213207, 213208, 213209, 213210, 213211, 213212, 213213, 213214, 213215, 213216, 213217, 213218) c6( ) c7(bL, dD, eR, e, aw, qQ, cz, aj, dG, j, v, eq, cd, J, ad, uT, Bc, iX, jy, dL, K, qM, bq, af, A, Mz, dq, iy, g, cM, aHG, bJ, D, c, hr, at, mf, ei, gT); #c1(UEVLD) c2(NP_001035787) c3(ubiquitin-conjugating enzyme E2 variant 3 isoform a) c4(33064) c5(33065, 33066, 33067, 33068, 33069, 33070) c6( ) c7(ci); #c1(UEVLD) c2(NP_001248311) c3(ubiquitin-conjugating enzyme E2 variant 3 isoform c) c4(93664) c5(93665, 93666, 93667, 93668, 93669, 93670) c6( ) c7(ci); #c1(UEVLD) c2(NP_001248313) c3(ubiquitin-conjugating enzyme E2 variant 3 isoform e) c4(93671) c5( ) c6( ) c7(ci); #c1(UEVLD) c2(NP_001284700) c3(ubiquitin-conjugating enzyme E2 variant 3 isoform h) c4(112335) c5(112336, 112337, 112338, 112339, 112340, 112341) c6( ) c7(ci); #c1(UEVLD) c2(NP_060784) c3(ubiquitin-conjugating enzyme E2 variant 3 isoform b) c4(197788) c5(197789, 197790, 197791, 197792, 197793, 197794) c6( ) c7(ci); #c1(UEVLD) c2(XP_011518500) c3(ubiquitin-conjugating enzyme E2 variant 3 isoform X1) c4( ) c5(312583) c6( ) c7(ci); #c1(UEVLD) c2(XP_011518503) c3(ubiquitin-conjugating enzyme E2 variant 3 isoform X4) c4( ) c5(312584, 312585, 312586, 312587, 312588) c6( ) c7(ci); #c1(UFD1L) c2(NP_001030324) c3(ubiquitin fusion degradation protein 1 homolog isoform B) c4( ) c5(31006, 31007, 31008, 31009, 31010, 31011) c6( ) c7(agd, cpq, RX, bFc, Az, bwE, ci, bD, agY, xg); #c1(UFD1L) c2(NP_005650) c3(ubiquitin fusion degradation protein 1 homolog isoform A) c4(158264, 158265, 158266, 158267) c5(158268, 158269, 158270, 158271, 158272, 158273, 158274, 158275, 158276, 158277, 158278, 158279, 158280, 158281, 158282, 158283, 158284, 158285) c6( ) c7(agd, cpq, RX, bFc, Az, bwE, ci, bD, agY, xg); #c1(UFL1) c2(NP_056138) c3(E3 UFM1-protein ligase 1) c4(185902) c5(185903, 185904, 185905, 185906, 185907, 185908) c6( ) c7(iu, T, ff, g, en, bz, B); #c1(UFM1) c2(NP_001273633) c3(ubiquitin-fold modifier 1 isoform 3) c4(105512) c5(105513, 105514, 105515, 105516, 105517, 105518) c6( ) c7(aW, al); #c1(UFM1) c2(NP_057701) c3(ubiquitin-fold modifier 1 isoform 1 precursor) c4(192448) c5(192449, 192450, 192451, 192452, 192453, 192454) c6( ) c7(aW, aT); #c1(UGCG) c2(NP_003349) c3(ceramide glucosyltransferase) c4(137701) c5(137702, 137703, 137704, 137705, 137706, 137707) c6( ) c7(bL, T, fd, bB, j, aT, ab, ey, K, af, d, aW, S, aER, UZ, ck, vl, bE, aGe, bzm, m, y, B); #c1(UGDH) c2(NP_001171629) c3(UDP-glucose 6-dehydrogenase isoform 2) c4(71026) c5(71027, 71028, 71029, 71030, 71031, 71032, 71033, 71034, 71035, 71036, 71037, 71038, 71039, 71040, 71041, 71042, 71043, 71044, 71045, 71046, 71047, 71048, 71049, 71050) c6( ) c7(dc, bo, aWJ, hD, jy, aq, xv, pw); #c1(UGDH) c2(NP_001171630) c3(UDP-glucose 6-dehydrogenase isoform 3) c4(71051) c5(71052, 71053, 71054, 71055, 71056) c6( ) c7(dc, bo, aWJ, hD, jy, aq, xv, pw); #c1(UGDH) c2(NP_003350) c3(UDP-glucose 6-dehydrogenase isoform 1) c4(137708) c5(137709, 137710, 137711, 137712, 137713, 137714, 137715, 137716, 137717, 137718, 137719, 137720, 137721, 137722, 137723, 137724, 137725, 137726, 137727) c6( ) c7(dc, bo, aWJ, hD, jy, aq, xv, pw); #c1(UGDH) c2(XP_005262724) c3(UDP-glucose 6-dehydrogenase isoform X1) c4(278056, 278057, 278058, 278059, 278060, 278061) c5(278062, 278063, 278064, 278065, 278066, 278067, 278068, 278069, 278070, 278071, 278072, 278073, 278074, 278075) c6( ) c7(dc, bo, aWJ, hD, jy, aq, xv, pw); #c1(UGGT1) c2(NP_064505) c3(UDP-glucose:glycoprotein glucosyltransferase 1 precursor) c4(202667) c5(202668, 202669, 202670, 202671, 202672, 202673, 202674, 202675, 202676, 202677, 202678, 202679) c6( ) c7(am, bqy, hC, ff, K, en, EB); #c1(UGGT1) c2(XP_006712697) c3(UDP-glucose:glycoprotein glucosyltransferase 1 isoform X1) c4(286810) c5( ) c6( ) c7(am, bqy, hC, ff, K, en, EB); #c1(UGGT2) c2(NP_064506) c3(UDP-glucose:glycoprotein glucosyltransferase 2 precursor) c4(202680) c5(202681, 202682, 202683, 202684, 202685, 202686) c6( ) c7(bL, ff, ac, en); #c1(UGGT2) c2(XP_011519403) c3(UDP-glucose:glycoprotein glucosyltransferase 2 isoform X8) c4(314050, 314051, 314052, 314053) c5(314054, 314055, 314056, 314057, 314058, 314059, 314060, 314061, 314062, 314063, 314064, 314065, 314066, 314067, 314068, 314069, 314070, 314071, 314072, 314073, 314074, 314075, 314076, 314077) c6( ) c7(bL, ff, ac, en); #c1(UGP2) c2(NP_006750) c3(UTP-glucose-1-phosphate uridylyltransferase isoform a) c4(167669, 167670, 167671, 167672, 167673, 167674, 167675, 167676, 167677, 167678) c5(167679, 167680, 167681, 167682, 167683, 167684, 167685, 167686, 167687, 167688, 167689, 167690, 167691, 167692, 167693, 167694, 167695, 167696, 167697, 167698, 167699, 167700, 167701, 167702, 167703, 167704, 167705, 167706, 167707, 167708, 167709) c6( ) c7(bt); #c1(UGP2) c2(XP_005264594) c3(UTP-glucose-1-phosphate uridylyltransferase isoform X1) c4( ) c5(279110, 279111, 279112, 279113, 279114, 279115, 279116, 279117, 279118, 279119, 279120, 279121, 279122, 279123, 279124, 279125, 279126, 279127, 279128, 279129, 279130, 279131, 279132, 279133, 279134, 279135, 279136, 279137, 279138, 279139, 279140, 279141, 279142) c6( ) c7(bt); #c1(UGT1A1) c2(NP_000454) c3(UDP-glucuronosyltransferase 1-1 precursor) c4(9144, 9145) c5(9146, 9147, 9148, 9149, 9150, 9151, 9152, 9153, 9154, 9155, 9156, 9157, 9158, 9159, 9160, 9161, 9162, 9163, 9164, 9165, 9166, 9167, 9168, 9169, 9170, 9171, 9172, 9173, 9174, 9175, 9176, 9177, 9178, 9179, 9180, 9181, 9182, 9183, 9184, 9185, 9186, 9187, 9188, 9189, 9190, 9191, 9192, 9193, 9194, 9195, 9196, 9197, 9198, 9199, 9200, 9201, 9202, 9203, 9204, 9205, 9206, 9207, 9208, 9209, 9210, 9211, 9212, 9213, 9214, 9215, 9216, 9217, 9218, 9219, 9220, 9221, 9222, 9223, 9224, 9225, 9226, 9227, 9228, 9229, 9230, 9231, 9232, 9233, 9234, 9235, 9236, 9237, 9238, 9239, 9240, 9241, 9242, 9243, 9244, 9245, 9246, 9247) c6(9248) c7(Mh, T, qo, fR, fV, if, e, bB, aE, u, fS, M, K, hu, eL, byv, bny, aW, hC, fc, hl, g, lm, eM, adl, uo, uJ, k, m, pw, cJJ, dD, sQ, aw, ht, NT, n, aj, Wb, fr, JW, cd, cHa, jz, Yc, byF, gd, h, eV, dL, cSO, bqy, AQ, cSP, fe, aq, anZ, hL, E, ut, ER, it, B, bL, wj, aCw, eX, rf, aA, tc, v, j, qf, C, q, J, aT, ae, eu, s, r, en, cl, t, aYI, hY, ck, BU, VJ, ac, hA, Yz, rA, ao, gV, qb, ha, Gn, eq, ad, av, avp, l, F, ds, oW, lQ, hG, cH, PE, Fm, ff, aid, aF, S, aot, iR, iV, dr, bF, es, qe, yE, bE, be, X, EB); #c1(UGTIA10) c2(NP_061948) c3(UDP-glucuronosyltransferase 1-10 precursor) c4(201258) c5(201259, 201260, 201261, 201262, 201263, 201264) c6( ) c7(bF, EB, T, jz, aW, ds, hC, bqy, ff, aT, r, aq, Yz, ci, en, q, dr); #c1(UGTIA3) c2(NP_061966) c3(UDP-glucuronosyltransferase 1-3 precursor) c4(201340) c5(201341, 201342, 201343, 201344, 201345, 201346, 201347) c6( ) c7(bF, EB, cJJ, byF, ds, hC, bqy, ff, adl, aq, Yz, en, hJ, dr); #c1(UGTIA4) c2(NP_009051) c3(UDP-glucuronosyltransferase 1-4 precursor) c4(170447) c5(170448, 170449, 170450, 170451, 170452, 170453) c6( ) c7(Mh, EB, bF, ds, hC, ha, bqy, ff, at, aq, dL, Yz, en, dr); #c1(UGTIA5) c2(NP_061951) c3(UDP-glucuronosyltransferase 1-5 precursor) c4(201279) c5(201280, 201281, 201282, 201283, 201284, 201285) c6( ) c7(ds, hC, bqy, ff, dr, en, EB); #c1(UGTIA6) c2(NP_001063) c3(UDP-glucuronosyltransferase 1-6 isoform 1 precursor) c4(34525, 34526, 34527, 34528, 34529) c5(34530, 34531, 34532, 34533, 34534, 34535, 34536, 34537, 34538, 34539, 34540, 34541) c6( ) c7(bM, T, ha, eX, n, j, q, byF, ds, aT, eV, ajV, B, su, s, hG, aW, bqy, hC, ff, r, aq, aB, en, bz, dr, bF, bE, Yz, Tu, m, X, EB); #c1(UGTIA6) c2(NP_995584) c3(UDP-glucuronosyltransferase 1-6 isoform 2) c4( ) c5(263958, 263959, 263960, 263961, 263962, 263963) c6( ) c7(bM, T, ha, eX, n, j, q, byF, ds, aT, eV, ajV, B, su, s, hG, aW, bqy, hC, ff, r, aq, aB, en, bz, dr, bF, bE, Yz, Tu, m, X, EB); #c1(UGTIA7) c2(NP_061950) c3(UDP-glucuronosyltransferase 1-7 precursor) c4(201272) c5(201273, 201274, 201275, 201276, 201277, 201278) c6( ) c7(gV, T, al, jx, aA, bB, hy, Gn, jE, ci, iG, q, eJ, hX, EB, aE, byF, ds, fS, gd, dL, eL, hG, bqy, hC, ff, r, aq, lo, aDd, en, iV, bz, dr, ar, gk, eM, bE, gX, adl, zl, rA, B, hv); #c1(UGTIA8) c2(NP_061949) c3(UDP-glucuronosyltransferase 1-8 precursor) c4(201265) c5(201266, 201267, 201268, 201269, 201270, 201271) c6( ) c7(EB, fR, hG, ff, aW, ds, fS, ht, hC, bqy, aT, r, n, oY, dL, aq, en, hu, it, dr); #c1(UGTIA9) c2(NP_066307) c3(UDP-glucuronosyltransferase 1-9 precursor) c4(207/76) c5(207/77, 207178, 207179, 207180, 207181, 207182, 207183) c6( ) c7(gV, T, bB, ci, q, ad, lv, ds, fS, aT, dL, B, hG, bqy, hC, ff, r, aq, en, lm, dr, eM, bE, E, uJ, lf, X, EB); #c1(UGT2A1) c2(NP_001239203) c3(UDP-glucuronosyltransferase 2A1 isoform 2 precursor) c4(88363) c5(88364, 88365, 88366, 88367, 88368, 88369) c6( ) c7(T); #c1(UGT2A1) c2(NP_001239204) c3(UDP-glucuronosyltransferase 2A1 isoform 3 precursor) c4(88370) c5(88371, 88372, 88373, 88374, 88375, 88376) c6( ) c7(T); #c1(UGT2A1) c2(NP_001288168) c3(UDP-glucuronosyltransferase 2A1 isoform 4 precursor) c4( ) c5(113672, 113673, 113674, 113675, 113676, 113677) c6( ) c7(T); #c1(UGT2A1) c2(NP_006789) c3(UDP-glucuronosyltransferase 2A1 isoform 1 precursor) c4(168083, 168084) c5(168085, 168086, 168087, 168088, 168089, 168090) c6( ) c7(T); #c1(UGT2A2) c2(NP_001099147) c3(UDP-glucuronosyltransferase 2A2 isoform UGT2A2_i1) c4(41804) c5(41805, 41806, 41807, 41808, 41809, 41810) c6( ) c7(T); #c1(UGT2A3) c2(NP_079019) c3(UDP-glucuronosyltransferase 2A3 precursor) c4(216602) c5(216603, 216604, 216605, 216606, 216607, 216608, 216609, 216610, 216611, 216612, 216613, 216614, 216615, 216616, 216617, 216618, 216619, 216620) c6( ) c7(hy, ne, BN); #c1(UGT2A3) c2(XP_011544634) c3(UDP-glucuronosyltransferase 2A3 isoform X1) c4(343753) c5(343754) c6( ) c7(hy, ne, BN); #c1(UGT2B10) c2(NP_001066) c3(UDP-glucuronosyltransferase 2810 isoform 1 precursor) c4(34549) c5(34550, 34551, 34552, 34553, 34554, 34555, 34556, 34557, 34558, 34559) c6( ) c7(PE, T, Y); #c1(UGT2B11) c2(NP_001064) c3(UDP-glucuronosyltransferase 2B11 precursor) c4(34542) c5(34543, 34544, 34545, 34546, 34547, 34548) c6( ) c7(Mb, aT); #c1(UGT2B15) c2(NP_001067) c3(UDP-glucuronosyltransferase 2815 precursor) c4(34560, 34561) c5(34562, 34563, 34564, 34565, 34566, 34567, 34568, 34569, 34570, 34571, 34572, 34573) c6( ) c7(bL, bF, T, cJJ, ad, byF, aW, Y, NT, al, r, n, aq, Yz, ae, q, LS, cp); #c1(UGT2B28) c2(NP_001193933) c3(UDP-glucuronosyltransferase 2828 isoform 2 precursor) c4(83236) c5(83237, 83238, 83239, 83240, 83241, 83242) c6( ) c7(q, r); #c1(UGT2B28) c2(NP_444267) c3(UDP-glucuronosyltransferase 2828 isoform 1 precursor) c4(230208) c5(230209, 230210, 230211, 230212, 230213, 230214) c6( ) c7(q, r); #c1(UGT2B4) c2(NP_001284544) c3(UDP-glucuronosyltransferase 284 isoform 2 precursor) c4(112035) c5(112036, 112037, 112038, 112039, 112040, 112041) c6( ) c7(bF, Mb, cJJ, kl, iI, aW, ht, aT, v, Yz, hu, it, B, bc); #c1(UGT2B4) c2(NP_001284545) c3(UDP-glucuronosyltransferase 284 isoform 3) c4(112042) c5(112043, 112044, 112045, 112046, 112047, 112048) c6( ) c7(bF, Mb, cJJ, kl, iI, aW, ht, aT, v, Yz, hu, it, B, bc); #c1(UGT2B4) c2(NP_066962) c3(UDP-glucuronosyltransferase 284 isoform 1 precursor) c4(207833) c5(207834, 207835, 207836, 207837, 207838, 207839) c6( ) c7(bF, Mb, cJJ, kl, iI, aW, ht, aT, v, Yz, hu, it, B, bc); #c1(UGT2B7) c2(NP_001065) c3(UDP-glucuronosyltransferase 287 precursor) c4( ) c5( ) c6( ) c7(gL, T, cJJ, id, VR, eR, qb, ha, n, aj, ci, bX, ad, byF, fS, cSQ, aT, h, dL, gl, aTX, eL, cp, hG, aW, r, aq, cl, iR, bF, es, iA, hL, Yz, B, hv); #c1(UGT3A2) c2(NP_001161788) c3(UDP-glucuronosyltransferase 3A2 isoform 2 precursor) c4(65994, 65995) c5(65996, 65997, 65998, 65999, 66000, 66001, 66002, 66003, 66004, 66005, 66006, 66007, 66008, 66009, 66010, 66011) c6(66012, 66013, 66014, 66015) c7(hy, ne, BN); #c1(UGT3A2) c2(NP_777574) c3(UDP-glucuronosyltransferase 3A2 isoform 1 precursor) c4(251178, 251179) c5(251180, 251181, 251182, 251183, 251184, 251185, 251186, 251187, 251188, 251189, 251190, 251191, 251192, 251193, 251194, 251195, 251196) c6(251197, 251198, 251199, 251200, 251201) c7(hy, ne, BN); #c1(UGT8) c2(NP_001121646) c3(2-hydroxyacylsphingosine 1-beta-galactosyltransferase precursor) c4(46009) c5( ) c6( ) c7(T, aC, pf, ha, fF, qN, aE, arA, sO, jZ, aT, Ps, s, aW, Y, qs, bz, fq, dR, ej, ar, qJ, nM, be); #c1(UGT8) c2(XP_006714366) c3(2-hydroxyacylsphingosine 1-beta-galactosyltransferase isoform X1) c4(287794) c5(287795, 287796, 287797, 287798, 287799, 287800, 287801, 287802, 287803, 287804, 287805, 287806, 287807, 287808, 287809, 287810) c6( ) c7(T, aC, pf, ha, fF, qN, aE, arA, sO, jZ, al, Ps, s, aW, Y, qs, bz, fq, dR, ej, ar, qJ, nM, be); #c1(UHMK1) c2(NP_001171692) c3(serine/threonine-protein kinase Kist isoform 2) c4(71314) c5(71315, 71316, 71317, 71318, 71319, 71320) c6( ) c7(ck, x, aW, aeO, gK, aT, aiz, bK, k, bw, bt); #c1(UHMK1) c2(NP_653225) c3(serine/threonine-protein kinase Kist isoform 3) c4(239185) c5(239186, 239187, 239188, 239189, 239190, 239191) c6( ) c7(ck, x, aW, ael, gK, aT, aiz, bK, k, bw, bt); #c1(UHMK1) c2(NP_787062) c3(serine/threonine-protein kinase Kist isoform 1) c4(252047) c5(252048, 252049, 252050, 252051, 252052, 252053) c6( ) c7(ck, x, aW, aeO, gK, aT, aiz, bk, k, bw, bt); #c1(UHRF1) c2(NP_001276980) c3(E3 ubiquitin-protein ligase UHRF1 isoform 1) c4( ) c5(108897, 108898, 108899, 108900, 108901, 108902, 108903, 108904, 108905, 108906, 108907, 108908, 108909, 108910, 108911, 108912, 108913) c6( ) c7(T, aA, bB, V, q, ad, eA, x, qK, aT, hn, dL, ae, eL, qw, aW, yx, r, aF, bz, es, ac, eM, nB, bE, iA, eo, k, aSs, B); #c1(UHRF1) c2(NP_037414) c3(E3 ubiquitin-protein ligase UHRF1 isoform 2) c4(175374, 175375, 175376) c5(175377, 175378, 175379, 175380, 175381, 175382, 175383, 175384, 175385, 175386) c6( ) c7(T, aA, bB, V, q, ad, eA, x, qK, aT, hn, dL, ae, eL, qw, aW, yx, r, aF, bz, es, ac, eM, nB, bE, iA, eo, k, aSs, B); #c1(UHRF1BP1) c2(NP_060224) c3(UHRF1-binding protein 1) c4(194840, 194841) c5(194842, 194843, 194844, 194845, 194846, 194847, 194848, 194849, 194850, 194851, 194852, 194853) c6( ) c7(aW, T, eZ, aT); #c1(UHRF2) c2(NP_690856) c3(E3 ubiquitin-protein ligase UHRF2) c4(244982, 244983) c5(244984, 244985, 244986, 244987, 244988, 244989, 244990) c6( ) c7(T, eM, aW, r, aT, bz, q); #c1(UHRF2) c2(XP_011516005) c3(E3 ubiquitin-protein ligase UHRF2 isoform X1) c4(309208) c5(309209, 309210, 309211, 309212, 309213, 309214, 309215) c6( ) c7(T, eM, aW, r, al, bz, q); #c1(UHRF2) c2(XP_011516007) c3(E3 ubiquitin-protein ligase UHRF2 isoform X2) c4( ) c5(309216, 309217, 309218, 309219, 309220) c6( ) c7(T, eM, aW, r, aT, bz, q); #c1(UIMC1) c2(NP_057374) c3(BRCA1-A complex subunit RAP80) c4(190426, 190427, 190428) c5(190429, 190430, 190431, 190432, 190433, 190434, 190435, 190436, 190437, 190438, 190439, 190440, 190441, 190442, 190443, 190444, 190445, 190446, 190447, 190448, 190449, 190450, 190451, 190452, 190453, 190454, 190455) c6( ) c7(T, eM, aW, al, gv, m, t, X); #c1(UIMC1) c2(XP_005265988) c3(BRCA1-A complex subunit RAP80 isoform X2) c4(279843) c5( ) c6( ) c7(T, eM, aW, aT, gv, m, t, X); #c1(UIMC1) c2(XP_005265990) c3(BRCA1-A complex subunit RAP80 isoform X7) c4( ) c5(279844, 279845, 279846, 279847, 279848, 279849) c6( ) c7(T, eM, aW, aT, gv, m, t, X); #c1(UIMC1) c2(XP_005265993) c3(BRCA1-A complex subunit RAP80 isoform X9) c4(279850) c5(279851, 279852, 279853, 279854, 279855, 279856) c6( ) c7(T, eM, aW, aT, gv, m, t, X); #c1(UIMC1) c2(XP_011532870) c3(BRCA1-A complex subunit RAP80 isoform X3) c4(329061, 329062) c5( ) c6( ) c7(T, eM, aW, aT, gv, m, t, X); #c1(UIMC1) c2(XP_011532872) c3(BRCA1-A complex subunit RAP80 isoform X6) c4( ) c5(329063, 329064, 329065, 329066, 329067, 329068) c6( ) c7(T, eM, aW, aT, gv, m, t, X); #c1(ULBP1) c2(NP_079494) c3(NKG2D ligand 1 precursor) c4(218367) c5(218368, 218369, 218370, 218371, 218372, 218373, 218374, 218375, 218376, 218377) c6( ) c7(ck, T, dA, bE, iK, jh, bB, n, h, gE, K, bt, bz, B); #c1(ULBP2) c2(NP_079493) c3(NKG2D ligand 2 preproprotein) c4(218359) c5(218360, 218361, 218362, 218363, 218364, 218365, 218366) c6( ) c7(ck, T, dA, ac, aE, bE, iU, Y, aA, h, fd, K); #c1(ULBP3) c2(NP_078794) c3(NKG2D ligand 3 precursor) c4(215491, 215492, 215493) c5(215494, 215495, 215496, 215497, 215498, 215499, 215500, 215501, 215502, 215503, 215504, 215505, 215506, 215507, 215508, 215509, 215510, 215511, 215512, 215513, 215514, 215515) c6( ) c7(VZ, OW, jh); #c1(ULK1) c2(NP_003556) c3(serine/threonine-protein kinase ULK1) c4(139174) c5(139175, 139176, 139177, 139178, 139179, 139180) c6( ) c7(T, eM, aW, aT, h, fe, K, it, eA); #c1(ULK1) c2(XP_011537101) c3(serine/threonine-protein kinase ULK1 isoform X2) c4( ) c5(334861) c6( ) c7(T, eM, aW, aT, h, fe, K, it, eA); #c1(ULK2) c2(NP_001136082) c3(serine/threonine-protein kinase ULK2) c4(54273) c5(54274, 54275, 54276, 54277, 54278, 54279, 54280, 54281, 54282, 54283) c6( ) c7(d, eA, an); #c1(ULK3) c2(NP_001092906) c3(serine/threonine-protein kinase ULK3 isoform a) c4(39764) c5(39765, 39766, 39767, 39768, 39769, 39770) c6( ) c7(iU, cz); #c1(ULK3) c2(XP_005254346) c3(serine/threonine-protein kinase ULK3 isoform X1) c4(273160) c5(273161, 273162, 273163, 273164, 273165, 273166) c6( ) c7(iU, cz); #c1(ULK4) c2(NP_060356) c3(serine/threonine-protein kinase ULK4) c4(195571, 195572) c5(195573, 195574, 195575, 195576, 195577, 195578) c6( ) c7(hb, oe, fd, cz, bK, K); #c1(ULK4) c2(XP_011532181) c3(serine/threonine-protein kinase ULK4 isoform X10) c4( ) c5(328217, 328218, 328219, 328220, 328221, 328222, 328223, 328224, 328225, 328226, 328227) c6( ) c7(hb, oe, fd, cz, bK, K); #c1(UMOD) c2(NP_001008390) c3(uromodulin isoform a preproprotein) c4(23145) c5( ) c6( ) c7(nD, cSS, dD, eR, ea, e, bKw, zj, cz, aj, vm, eq, aVK, aY, vN, J, fU, blk, nH, cSR, aT, h, bKs, K, bq, uD, bA, sj, A, bKt, bKv, qa, hq, lI, fP, hO, lO, dy, cgP, aAN); #c1(UMOD) c2(NP_001265543) c3(uromodulin isoform b preproprotein) c4(98792) c5(98793, 98794, 98795, 98796, 98797, 98798, 98799, 98800, 98801, 98802) c6( ) c7(nD, cSS, dD, eR, ea, e, bKw, zj, cz, aj, vm, eq, aVK, aY, vN, J, fU, blk, nH, cSR, aT, h, bKs, K, bq, uD, bA, sj, A, bKt, bKv, qa, hq, lI, fP, hO, lO, dy, cgP, aAN); #c1(UMOD) c2(XP_011544241) c3(uromodulin isoform X3) c4(343379) c5(343380, 343381, 343382, 343383, 343384, 343385, 343386, 343387, 343388, 343389, 343390, 343391, 343392, 343393, 343394, 343395, 343396, 343397) c6( ) c7(nD, cSS, dD, eR, ea, e, bKw, zj, cz, aj, vm, eq, aVK, aY, vN, J, fU, blk, nH, cSR, aT, h, bKs, K, bq, uD, bA, sj, A, bKt, bKv, qa, hq, lI, fP, hO, lO, dy, cgP, aAN); #c1(UMOD) c2(XP_011544242) c3 (uromodulin isoform X4) c4( ) c5(343398, 343399) c6( ) c7(nD, cSS, dD, eR, ea, e, bKw, zj, cz, aj, vm, eq, aVK, aY, vN, J, fU, blk, nH, cSR, aT, h, bKs, K, bq, uD, bA, sj, A, bKt, bKv, qa, hq, lI, fP, hO, lO, dy, cgP, aAN); #c1(UMODL1) c2(NP_001004416) c3(uromodulin-like 1 isoform 1 precursor) c4(20659, 20660, 20661) c5(20662, 20663, 20664, 20665, 20666, 20667, 20668, 20669, 20670, 20671, 20672, 20673, 20674, 20675) c6( ) c7(Be); #c1(UMODL1) c2(NP_775839) c3(uromodulin-like 1 isoform 2 precursor) c4(250476) c5(250477, 250478, 250479, 250480, 250481, 250482, 250483, 250484) c6( ) c7(Be); #c1(UMODL1) c2(XP_011528090) c3(uromodulin-like 1 isoform X1) c4(323167) c5(323168, 323169, 323170, 323171, 323172) c6( ) c7(Be); #c1(UMODL1) c2(XP_011528091) c3(uromodulin-like 1 isoform X2) c4(323173) c5( ) c6( ) c7(Be); #c1(UMODL1) c2(XP_011528097) c3(uromodulin-like 1 isoform X3) c4(323174) c5( ) c6( ) c7(Be); #c1(UMODL1) c2(XP_011528102) c3(uromodulin-like 1 isoform X7) c4( ) c5(323175, 323176) c6( ) c7(Be); #c1(UMODL1) c2(XP_011528104) c3(uromodulin-like 1 isoform X8) c4(323177) c5(323178, 323179, 323180, 323181, 323182, 323183) c6( ) c7(Be); #c1(UMODL1) c2(XP_011528105) c3(uromodulin-like 1 isoform X9) c4(323184) c5(323185, 323186, 323187, 323188, 323189, 323190, 323191, 323192, 323193, 323194, 323195, 323196, 323197, 323198) c6( ) c7(Be); #c1(UMPS) c2(NP_000364) c3(uridine 5′-monophosphate synthase) c4(8119, 8120, 8121, 8122) c5(8123, 8124, 8125, 8126, 8127, 8128, 8129, 8130, 8131, 8132, 8133, 8134, 8135, 8136, 8137, 8138, 8139, 8140, 8141, 8142, 8143, 8144, 8145, 8146) c6( ) c7(gV, G, T, aC, qb, aA, n, awr, cnk, ci, q, ad, gd, aT, dV, bq, CK, B, hG, hC, ff, r, en, g, bz, VJ, eM, pw, aws, X); #c1(UNC119) c2(NP_005139) c3(protein unc-119 homolog A isoform a) c4(153330) c5(153331, 153332, 153333, 153334, 153335, 153336) c6( ) c7(IR, aqV, nw, nC, aJc, aa); #c1(UNC(19) c2(NP_473376) c3(protein unc-119 homolog A isoform b) c4(230511, 230512) c5(230513, 230514, 230515, 230516, 230517, 230518, 230519, 230520, 230521, 230522, 230523, 230524) c6( ) c7(IR, aqV, nw, nC, aJc, aa); #c1(UNC13A) c2(NP_001073890) c3(protein unc-13 homolog A) c4(36316, 36317, 36318) c5(36319, 36320, 36321, 36322, 36323, 36324, 36325, 36326, 36327, 36328, 36329, 36330, 36331, 36332, 36333, 36334, 36335, 36336) c6( ) c7(bb, dG, Vd, c); #c1(UNC13B) c2(NP_006368) c3(protein unc-13 homolog B) c4(164410) c5(164411, 164412, 164413, 164414, 164415, 164416) c6( ) c7(A, anp, an, dG, dy, pz); #c1(UNC13B) c2(XP_011515983) c3(protein unc-13 homolog B isoform X1) c4(309155) c5(309156) c6( ) c7(A, anp, an, dG, dy, pz); #c1(UNC13B) c2(XP_011515986) c3(protein unc-13 homolog B isoform X4) c4(309157, 309158) c5(309159, 309160, 309161, 309162, 309163, 309164, 309165, 309166, 309167, 309168, 309169, 309170) c6( ) c7(A, anp, an, dG, dy, pz); #c1(UNC13B) c2(XP_011515988) c3(protein unc-13 homolog B isoform X6) c4( ) c5(309171, 309172, 309173, 309174, 309175, 309176) c6( ) c7(A, anp, an, dG, dy, pz); #c1(UNC13C) c2(NP_001074003) c3(protein unc-13 homolog C) c4(36916, 36917, 36918) c5(36919, 36920, 36921, 36922, 36923, 36924, 36925, 36926, 36927, 36928, 36929, 36930, 36931, 36932, 36933, 36934, 36935, 36936) c6( ) c7(e, oe, bX, an, fq, cd); #c1(UNC13D) c2(NP_954712) c3 (protein unc-13 homolog D) c4(261333, 261334, 261335) c5(261336, 261337, 261338, 261339, 261340, 261341, 261342, 261343, 261344, 261345, 261346, 261347, 261348, 261349, 261350, 261351, 261352, 261353) c6( ) c7(gV, T, bxK, hG, aW, Bj, aT, bQc, aq, cl, zA, mK, pz, iR, hF, cST, ai, o); #c1 (UNC45A) c2(NP_001034764) c3(protein unc-45 homolog A isoform 3) c4(32272) c5(32273, 32274, 32275, 32276, 32277, 32278) c6( ) c7(cm, aW, ci, al); #c1(UNC45A) c2(NP_061141) c3(protein unc-45 homolog A isoform 2) c4(199505) c5(199506, 199507, 199508, 199509, 199510, 199511) c6( ) c7(cm, aW, ci, aT); #c1(UNC45B) c2(NP_001253981) c3(protein unc-45 homolog B isoform 2) c4(94354) c5(94355, 94356, 94357, 94358, 94359, 94360, 94361) c6( ) c7(gZ, cc, K, Az); #c1(UNC45B) c2(NP_775259) c3(protein unc-45 homolog B isoform 1) c4(249680) c5(249681, 249682, 249683, 249684, 249685, 249686, 249687, 249688, 249689, 249690) c6( ) c7(gZ, cc, K, Az); #c1(UNC5A) c2(NP_588610) c3(netrin receptor UNC5A precursor) c4(234785, 234786) c5(234787, 234788, 234789, 234790, 234791, 234792, 234793, 234794, 234795, 234796, 234797, 234798) c6( ) c7(eL, dL); #c1(UNC5B) c2(NP_001231818) c3(netrin receptor UNC5B isoform 2 precursor) c4(87564) c5(87565, 87566, 87567, 87568, 87569, 87570) c6( ) c7(jf, T, dD, iA, ea, eL, r, n, dL, eq, K, fr, y, ae, q); #c1(UNC5B) c2(NP_734465) c3(netrin receptor UNC5B isoform 1 precursor) c4(247292) c5(247293, 247294, 247295, 247296, 247297, 247298) c6( ) c7(jf, T, dD, iA, ea, eL, r, n, dL, eq, K, fr, y, ae, q); #c1(UNC5C) c2(NP_003719) c3(netrin receptor UNC5C precursor) c4(140390, 140391) c5(140392, 140393, 140394, 140395, 140396, 140397, 140398, 140399, 140400, 140401, 140402, 140403, 140404) c6( ) c7(T, iA, bE, Y, go, aw, r, aA, bB, v, aF, ci, fr, an, y, q, de); #c1(UNC5D) c2(NP_543148) c3(netrin receptor UNC5D precursor) c4(233465, 233466) c5(233467, 233468, 233469, 233470, 233471, 233472, 233473, 233474, 233475, 233476, 233477, 233478) c6( ) c7(cm, es, fr, ci, iA); #c1(UNC5D) c2(XP_011542706) c3(netrin receptor UNC5D isoform X8) c4( ) c5(341259) c6( ) c7(cm, es, fr, ci, iA); #c1(UNC5D) c2(XP_011542707) c3(netrin receptor UNC5D isoform X9) c4( ) c5(341260) c6( ) c7(cm, es, fr, ci, iA); #c1(UNC79) c2(NP_065869) c3(protein unc-79 homolog) c4(205958) c5(205959, 205960, 205961, 205962, 205963) c6( ) c7(oe); #c1(UNC79) c2(XP_011544953) c3(protein unc-79 homolog isoform X3) c4(343945) c5(343946, 343947, 343948, 343949, 343950, 343951) c6( ) c7(oe); #c1(UNC79) c2(XP_011544954) c3(protein unc-79 homolog isoform X4) c4(343952, 343953) c5(343954, 343955, 343956, 343957, 343958, 343959, 343960, 343961, 343962, 343963, 343964, 343965) c6( ) c7(oe); #c1(UNC79) c2(XP_011544956) c3(protein unc-79 homolog isoform X6) c4( ) c5(343966) c6( ) c7(oe); #c1(UNC79) c2(XP_011544962) c3(protein unc-79 homolog isoform X11) c4(343967, 343968, 343969) c5( ) c6( ) c7(De); #c1(UNC93A) c2(NP_001137419) c3(protein unc-93 homolog A isoform 2) c4(55665) c5(55666, 55667, 55668, 55669, 55670, 55671) c6( ) c7(t); #c1(UNC93A) c2(XP_011534209) c3(protein unc-93 homolog A isoform X1) c4(330534) c5(330535, 330536, 330537, 330538, 330539, 330540) c6( ) c7(t); #c1(UNC93A) c2(XP_011534210) c3(protein unc-93 homolog A isoform X2) c4(330541) c5( ) c6( ) c7(t); #c1(UNC93A) c2(XP_011534211) c3(protein unc-93 homolog A isoform X3) c4( ) c5(330542, 330543, 330544, 330545, 330546, 330547) c6( ) c7(t); #c1(UNC93B1) c2(NP_112192) c3(protein unc-93 homolog B1) c4(220724) c5(220725, 220726, 220727, 220728, 220729, 220730) c6( ) c7(xD, eW, cSU, yx, hh); #c1(UNC93B1) c2(XP_011543592) c3(protein unc-93 homolog B1 isoform X1) c4(342508) c5( ) c6( ) c7(xD, eW, cSU, yx, hh); #c1(UNC93B1) c2(XP_011543593) c3(protein unc-93 homolog B1 isoform X2) c4( ) c5(342509, 342510, 342511, 342512, 342513, 342514, 342515, 342516, 342517) c6( ) c7(xD, eW, cSU, yx, hh); #c1(UNG) c2(NP_003353) c3(uracil-DNA glycosylase isoform UNG1 precursor) c4(137728) c5(137729, 137730, 137731, 137732, 137733, 137734, 137735, 137736, 137737, 137738, 137739, 137740) c6( ) c7(T, dA, ew, jh, bB, gA, hF, q, cSV, ad, De, x, eW, h, dL, K, ae, afu, d, iU, Cw, oV, zb, ff, r, en, bz, ck, ac, bE, U, k, bt, zA); #c1(UNG) c2(NP_550433) c3(uracil-DNA glycosylase isoform UNG2) c4(233486, 233487) c5(233488, 233489, 233490, 233491, 233492, 233493) c6( ) c7(T, dA, ew, jh, bB, gA, hF, q, cSV, ad, De, x, eW, h, dL, K, ae, afu, d, iU, Cw, oV, zb, ff, r, en, bz, ck, ac, bE, U, k, bt, zA); #c1(UPB1) c2(NP_057411) c3(beta-ureidopropionase) c4(190612, 190613, 190614) c5(190615, 190616, 190617, 190618, 190619, 190620, 190621, 190622, 190623, 190624, 190625, 190626) c6( ) c7(dq, cC, cd, bN, cSW); #c1(UPB1) c2(XP_011528526) c3(beta-ureidopropionase isoform X3) c4( ) c5(323665, 323666, 323667, 323668, 323669, 323670) c6( ) c7(dq, cC, cd, bN, cSW); #c1(UPF1) c2(NP_002902) c3(regulator of nonsense transcripts 1 isoform 2) c4(133437) c5(133438, 133439, 133440, 133441, 133442, 133443) c6( ) c7(T, cSX, aA, r, ci, bC, ae, ad); #c1(UPF2) c2(NP_542166) c3(regulator of nonsense transcripts 2) c4(232142) c5(232143, 232144, 232145, 232146, 232147, 232148, 232149, 232150, 232151, 232152, 232153, 232154, 232155, 232156, 232157) c6( ) c7(c, ad, nA, r); #c1(UPF2) c2(XP_011517750) c3(regulator of nonsense transcripts 2 isoform X1) c4(311570, 311571) c5(311572, 311573, 311574, 311575, 311576) c6( ) c7(c, ad, nA, r); #c1(UPF2) c2(XP_011517751) c3(regulator of nonsense transcripts 2 isoform X2) c4( ) c5(311577, 311578, 311579, 311580, 311581, 311582, 311583, 311584, 311585, 311586) c6( ) c7(c, ad, nA, r); #c1(UPF3A) c2(NP_075387) c3(regulator of nonsense transcripts 3A isoform hUpf3p) c4(213834, 213835) c5(213836, 213837, 213838, 213839, 213840, 213841, 213842) c6( ) c7(c); #c1(UPF3A) c2(NP_542418) c3(regulator of nonsense transcripts 3A isoform hUpf3pdelta) c4(232830) c5(232831, 232832, 232833, 232834, 232835, 232836) c6( ) c7(c); #c1(UPF3A) c2(XP_011533150) c3(regulator of nonsense transcripts 3A isoform X5) c4( ) c5(329295, 329296, 329297, 329298, 329299) c6( ) c7(c); #c1(UPF3B) c2(NP_075386) c3(regulator of nonsense transcripts 38 isoform 2) c4(213820, 213821) c5(213822, 213823, 213824, 213825, 213826, 213827, 213828, 213829, 213830, 213831, 213832, 213833) c6( ) c7(cSY, nf, nA, QK, bN, bKE); #c1(UPF3B) c2(NP_542199) c3(regulator of nonsense transcripts 38 isoform 1) c4(232280) c5(232281, 232282, 232283, 232284, 232285, 232286) c6( ) c7(cSY, nf, nA, QK, bN, bKE); #c1(UPK1A) c2(NP_001268372) c3 (uroplakin-la isoform 2) c4(99803) c5(99804, 99805, 99806, 99807, 99808, 99809) c6( ) c7(T, id, wG, Jl, aA, aF, it); #c1(UPK1A) c2(NP_008931) c3 (uroplakin-1a isoform 1) c4(169633) c5(169634, 169635, 169636, 169637, 169638, 169639) c6( ) c7(T, id, wG, Jl, aA, aF, it); #c1(UPK1B) c2(NP_008883) c3(uroplakin-1b) c4(169491, 169492, 169493) c5(169494, 169495, 169496, 169497, 169498, 169499, 169500, 169501, 169502, 169503, 169504, 169505, 169506, 169507, 169508, 169509, 169510, 169511) c6( ) c7(eL, T, dt, id, ci); #c1(UPK2) c2(NP_006751) c3(uroplakin-2 precursor) c4(167710) c5(167711, 167712, 167713, 167714, 167715, 167716) c6( ) c7(XX, T, id, ff, aJN, dL, en, eL); #c1(UPK3A) c2(NP_001161046) c3(uroplakin-3a isoform 2 precursor) c4(65201) c5(65202, 65203, 65204, 65205, 65206, 65207) c6( ) c7(id, wG, lI, n, dL, lN, bX); #c1(UPK3A) c2(NP_008884) c3(uroplakin-3a isoform 1 precursor) c4(169512) c5(169513, 169514, 169515, 169516, 169517, 169518) c6( ) c7(id, wG, lI, n, dL, lN, bX); #c1(UPP1) c2(NP_001274355) c3(uridine phosphorylase 1 isoform a) c4(106239, 106240, 106241, 106242) c5(106243, 106244, 106245, 106246, 106247, 106248, 106249, 106250, 106251, 106252, 106253, 106254, 106255, 106256) c6( ) c7(ck, T, wi, aW, Jp, fJ, aT, r, ci, m, t, q); #c1(UPP1) c2(NP_001274357) c3(uridine phosphorylase 1 isoform b) c4(106269) c5(106270, 106271, 106272, 106273, 106274, 106275) c6( ) c7(ck, T, wi, aW, Jp, fJ, aT, r, ci, m, t, q); #c1(UPP1) c2(XP_006715834) c3(uridine phosphorylase 1 isoform X5) c4(289842) c5(289843, 289844, 289845, 289846, 289847, 289848, 289849, 289850, 289851, 289852, 289853, 289854, 289855, 289856, 289857, 289858, 289859, 289860, 289861, 289862, 289863, 289864, 289865, 289866) c6( ) c7(ck, T, wi, aW, Jp, fJ, aT, r, ci, m, t, q); #c1(UPP2) c2(NP_001128570) c3(uridine phosphorylase 2 isoform b) c4(50273, 50274) c5(50275, 50276, 50277, 50278, 50279, 50280, 50281) c6( ) c7(T, id, VX, ff, en, bN); #c1(UPP2) c2(NP_775491) c3(uridine phosphorylase 2 isoform a) c4( ) c5(250091, 250092, 250093, 250094, 250095, 250096, 250097) c6(250098, 250099) c7(T, id, VX, ff, en, bN); #c1(UPRT) c2(NP_659489) c3(uracil phosphoribosyltransferase homolog) c4(240179) c5(240180, 240181, 240182, 240183, 240184, 240185) c6( ) c7(ck, T, aE, bE, r, bB, m, eA, bq, CQ, t, iK, q); #c1(UACC1) c2(NP_001171906) c3(ubiquinol-cytochrome-c reductase complex assembly factor 1 isoform c) c4(72012) c5(72013, 72014, 72015, 72016, 72017, 72018) c6( ) c7(aqe); #c1(UQCC1) c2(NP_060714) c3(ubiquinol-cytochrome-c reductase complex assembly factor 1 isoform a) c4(197402, 197403, 197404) c5(197405, 197406, 197407, 197408, 197409, 197410, 197411, 197412, 197413, 197414, 197415, 197416) c6( ) c7(aqe); #c1(UQCC1) c2(NP_955781) c3(ubiquinol-cytochrome-c reductase complex assembly factor 1 isoform b) c4(261967) c5(261968, 261969, 261970, 261971, 261972, 261973, 261974, 261975, 261976, 261977, 261978, 261979) c6( ) c7(aqe); #c1(UQCC1) c2(XP_011527180) c3(ubiquinol-cytochrome-c reductase complex assembly factor 1 isoform X2) c4(321690, 321691, 321692, 321693) c5(321694) c6( ) c7(aqe); #c1(UQCC1) c2(XP_011527183) c3(ubiquinol-cytochrome-c reductase complex assembly factor 1 isoform X4) c4( ) c5(321695, 321696, 321697, 321698, 321699, 321700) c6( ) c7(aqe); #c1(UQCC1) c2(XP_011527186) c3(ubiquinol-cytochrome-c reductase complex assembly factor 1 isoform X5) c4( ) c5(321701, 321702, 321703, 321704, 321705, 321706, 321707, 321708, 321709, 321710, 321711, 321712, 321713, 321714, 321715, 321716, 321717, 321718) c6( ) c7(aqe); #c1(UQCC2) c2(NP_115716) c3(ubiquinol-cytochrome-c reductase complex assembly factor 2) c4(223694, 223695) c5(223696, 223697, 223698, 223699, 223700, 223701, 223702, 223703, 223704, 223705, 223706, 223707) c6( ) c7(aqe, kl, eZ); #c1 (UACRB) c2(NP_006285) c3(cytochrome b-c1 complex subunit 7 isoform 1) c4(163593) c5(163594, 163595, 163596, 163597, 163598, 163599) c6( ) c7(aqb, SE, ad, bo, aqe); #c1(UACRC1) c2(NP_003356) c3(cytochrome b-c1 complex subunit 1, mitochondrial precursor) c4(137748) c5(137749, 137750, 137751, 137752, 137753, 137754) c6( ) c7(t); #c1(UQCRC2) c2(NP_003357) c3(cytochrome b-c1 complex subunit 2, mitochondrial precursor) c4(137755) c5(137756, 137757, 137758, 137759, 137760, 137761) c6( ) c7(cSZ, v, cF); #c1(UQCRFS1) c2(NP_005994) c3(cytochrome b-c1 complex subunit Rieske, mitochondrial) c4(161084) c5(161085, 161086, 161087, 161088, 161089, 161090) c6( ) c7(T, eG, d, W, eM, aW, nI, aT, r, j, ci, K, m, iK, q, ad); #c1(UACRQ) c2(NP_055217) c3(cytochrome b-c1 complex subunit 8) c4(179897) c5(179898, 179899, 179900, 179901, 179902, 179903, 179904, 179905, 179906, 179907, 179908, 179909, 179910, (79911) c6( ) c7(aqb, cTa, SE, bo, aqe); #c1(URB2) c2(XP_005273417) c3(unhealthy ribosome biogenesis protein 2 homolog isoform X2) c4( ) c5(284579) c6( ) c7(De); #c1(URGCP) c2(NP_001071131) c3(up-regulator of cell proliferation isoform 3) c4(35308, 35309, 35310) c5(35311, 35312, 35313, 35314, 35315, 35316, 35317, 35318, 35319) c6( ) c7(eH, aA, eF, cm, B, hX); #c1(URGCP) c2(NP_001277005) c3(up-regulator of cell proliferation isoform 2) c4(108943) c5(108944, 108945, 108946, 108947, 108948, 108949, 108950, 108951, 108952, 108953, 108954, 108955, 108956, 108957, 108958, 108959, 108960, 108961, 108962, 108963, 108964, 108965, 108966, 108967) c6( ) c7(eH, aA, eF, cm, B, hX); #c1(URGCP) c2(NP_060390) c3(up-regulator of cell proliferation isoform 1) c4(195781) c5(195782, 195783, 195784, 195785, 195786, 195787, 195788, 195789, 195790) c6( ) c7(eH, aA, eF, cm, B, hX); #c1(UR11) c2(NP_001239570) c3(unconventional prefoldin RPB5 interactor 1 isoform c) c4(88552) c5(88553, 88554, 88555, 88556, 88557, 88558) c6( ) c7(ov, cZ, bM, ig, dB, On, eX, bDJ, eV, bDI, m, ci, dd, pL, t, B, y, hX); #c1(UR11) c2(NP_003787) c3(unconventional prefoldin RPB5 interactor 1 isoform a) c4(140797) c5(140798, 140799, 140800, 140801, 140802, 140803, 140804) c6( ) c7(ov, cZ, bM, ig, dB, On, eX, bDJ, eV, bDI, m, ci, dd, pL, t, B, y, hX); #c1(UR11) c2(XP_005259419) c3(unconventional prefoldin RPB5 interactor 1 isoform X1) c4(276093, 276094) c5( ) c6( ) c7(ov, cZ, bM, ig, dB, On, ex, bDJ, eV, bDI, m, ci, dd, pL, t, B, y, hX); #c1(UR11) c2(XP_011525737) c3(unconventional prefoldin RPB5 interactor 1 isoform X3) c4( ) c5(320140, 320141, 320142, 320143, 320144, 320145, 320146, 320147, 320148, 320149, 320150) c6( ) c7(ov, cZ, bM, ig, dB, On, ex, bDJ, eV, bDI, m, ci, dd, pL, t, B, y, hX); #c1(URM1) c2(NP_001129419) c3(ubiquitin-related modifier 1 isoform b) c4(51467) c5(51468, 51469, 51470, 51471, 51472, 51473, 51474, 51475) c6( ) c7(aA); #c1(URM1) c2(NP_001252511) c3(ubiquitin-related modifier 1 isoform c) c4( ) c5(94042, 94043, 94044, 94045, 94046, 94047, 94048, 94049, 94050) c6( ) c7(aA); #c1(URM1) c2(NP_112176) c3(ubiquitin-related modifier 1 isoform a) c4(220624, 220625) c5(220626, 220627, 220628, 220629, 220630, 220631) c6( ) c7(aA); #c1(UROC1) c2(NP_001159446) c3(urocanate hydratase isoform 2) c4(64084) c5(64085, 64086, 64087, 64088, 64089, 64090) c6( ) c7(cTc, cTb, nA); #c1(UROC1) c2(NP_653240) c3 (urocanate hydratase isoform 1) c4(239273) c5(239274, 239275, 239276, 239277, 239278, 239279) c6( ) c7(cTc, cTb, nA); #c1(UROD) c2(XP_011540382) c3(uroporphyrinogen decarboxylase isoform X1) c4(338869) c5(338870, 338871, 338872, 338873, 338874, 338875, 338876, 338877, 338878, 338879) c6( ) c7(T, btY, QW, jx, mO, aHv, aCm, bS, hy, ct, C, rc, bbB, qC, MP, nI, cTf, yu, cTh, lQ, cTg, rz, fb, bsq, iz, Nu, vT, LO, hl, nh, yy, cTd, dq, Z, dr, bN, OC, iO, cTe, agl, QV, abX, aCo, hE, KY); #c1(UROS) c2(NP_000366) c3 (uroporphyrinogen-III synthase) c4(8147, 8148) c5(8149, 8150, 8151, 8152, 8153, 8154, 8155, 8156, 8157, 8158) c6( ) c7(mO, nI, cTi, dq, Km, S, dr); #c1(UROS) c2(XP_005270195) c3(uroporphyrinogen-III synthase isoform X2) c4(283243) c5( ) c6( ) c7(mO, nI, cli, dq, Km, S, dr); #c1(UROS) c2(XP_005270197) c3(uroporphyrinogen-III synthase isoform X6) c4( ) c5(283244, 283245, 283246, 283247, 283248, 283249) c6( ) c7(mO, nI, cli, dq, Km, S, dr); #c1(UROS) c2(XP_005270198) c3(uroporphyrinogen-III synthase isoform X7) c4(283250) c5(283251, 283252, 283253, 283254, 283255, 283256, 283257, 283258, 283259, 283260, 283261, 283262) c6( ) c7(mO, nI, cTi, dq, Km, S, dr); #c1(USB1) c2(NP_001182231) c3(U6 snRNA phosphodiesterase isoform 2) c4(74725) c5(74726, 74727, 74728, 74729, 74730, 74731) c6( ) c7(sb, gV, hG, aVA, lQ, ask); #c1(USB1) c2(NP_001191840) c3(U6 snRNA phosphodiesterase isoform 3) c4(81848) c5(81849, 81850, 81851, 81852, 81853, 81854) c6( ) c7(sb, gV, hG, aVA, lQ, ask); #c1(USB1) c2(NP_078874) c3(U6 snRNA phosphodiesterase isoform 1) c4(215861, 215862) c5(215863, 215864, 215865, 215866, 215867, 215868) c6( ) c7(sb, gV, hG, aVA, lQ, ask); #c1(USB1) c2(XP_011521630) c3(U6 snRNA phosphodiesterase isoform X1) c4( ) c5(316425, 316426, 316427, 316428, 316429, 316430) c6( ) c7(sb, gV, hG, aVA, lQ, ask); #c1(USE1) c2(NP_060937) c3(vesicle transport protein USE1) c4(198784, 198785, 198786) c5(198787, 198788, 198789, 198790, 198791, 198792, 198793, 198794, 198795, 198796, 198797, 198798, 198799, 198800, 198801, 198802, 198803, 198804, 198805) c6( ) c7(T, ib, hX, aW, acu, gC, cr, aT, fd, jh, gu, m, t, hq); #c1(USF1) c2(NP_001263302) c3(upstream stimulatory factor 1 isoform 1) c4(97126, 97127, 97128) c5(97129, 97130, 97131, 97132, 97133, 97134, 97135, 97136, 97137, 97138, 97139, 97140, 97141, 97142, 97143, 97144, 97145, 97146) c6( ) c7(bL, T, dD, ea, e, bB, aj, dG, j, ci, v, eq, J, TB, av, Mo, hd, dY, ec, aT, K, dQ, af, kj, cW, aW, eZ, xH, Ku, g, bF, dC, nB, bE, Fi, eM, ei, Zw, L); #c1(USF1) c2(NP_996888) c3(upstream stimulatory factor 1 isoform 2) c4( ) c5(264810, 264811, 264812, 264813, 264814) c6( ) c7(bL, T, dD, ea, e, bB, aj, dG, j, ci, v, eq, J, TB, av, Mo, hd, dY, ec, aT, K, dQ, af, kj, cW, aW, eZ, xH, Ku, g, bF, dC, nB, bE, Fi, eM, ei, Zw, L); #c1(USF2) c2(NP_003358) c3(upstream stimulatory factor 2 isoform 1) c4(137762) c5(137763, 137764, 137765, 137766, 137767, 137768, 137769) c6( ) c7(gh, G, T, n, aoM, ci, q, ad, aFu, dP, ft, fX, hd, aT, K, ae, af, iI, aW, ff, r, aq, Fq, Ku, en, g, bw, bF, nB, wR, B); #c1(USF2) c2(NP_997174) c3(upstream stimulatory factor 2 isoform 2) c4(265061, 265062) c5(265063, 265064, 265065, 265066, 265067, 265068, 265069, 265070, 265071, 265072, 265073, 265074) c6( ) c7(gh, G, T, n, aoM, ci, q, ad, aFu, dP, ft, fX, hd, aT, K, ae, af, iI, aW, ff, r, aq, Fq, Ku, en, g, bw, bF, nB, wR, B); #c1(USH1C) c2(NP_001284693) c3(harmonin isoform c) c4(112328) c5(112329, 112330, 112331, (12332, 112333, 112334) c6( ) c7(alx, cTk, lR, bE, mG, bB, oc, nw, mc, nk, cTj, Bh, q, bCJ); #c1(USH1C) c2(NP_005700) c3(harmonin isoform a) c4(158640, 158641) c5(158642, 158643, 158644, 158645, 158646, 158647, 158648, 158649, 158650, 158651, 158652, 158653) c6( ) c7(alx, cTk, lR, bE, mG, bB, oc, nw, mc, nk, cOj, Bh, q, bCJ); #c1(USH1C) c2(NP_710142) c3(harmonin isoform b3) c4(246472) c5(246473, 246474, 246475, 246476, 246477, 246478, 246479, 246480) c6( ) c7(alx, cTk, lR, bE, mG, bB, oc, nw, mc, nk, cTj, Bh, q, bCJ); #c1(USH1G) c2(NP_775748) c3(Usher syndrome type-1G protein isoform 1) c4(250161) c5(250162, 250163, 250164, 250165, 250166, 250167) c6( ) c7(iH, alx, lR, dA, bdp, Xz, bS, ct, nw, coA, Z, oc); #c1(USH2A) c2(NP_009054) c3(usherin isoform A) c4( ) c5(170469, 170470, 170471, 170472, 170473, 170474) c6( ) c7(alx, lR, xl, mG, cTl, FQ, nw, Nh, nk, rz, nC, cpk, oc, dr, ad); #c1(USH2A) c2(NP_996816) c3(usherin isoform B) c4(264646, 264647, 264648) c5(264649, 264650, 264651, 264652, 264653, 264654, 264655, 264656, 264657, 264658, 264659, 264660, 264661, 264662, 264663, 264664) c6( ) c7(alx, lR, xl, mG, cTl, FR, nw, Nh, nk, rz, nC, cpk, oc, dr, ad); #c1(USO1) c2(NP_001276978) c3(general vesicular transport factor p115 isoform 1) c4(108890) c5(108891, 108892, 108893, 108894, 108895, 108896) c6( ) c7(oO, T, bvt, wL, if, jx, aA, eb, n, cl, fr, bvx, ci, qN, V, eW, o, ad, hX, jF, av, F, dY, h, K, lQ, hu, hH, bA, aa, uQ, A, mR, dH, Y, KS, cr, eZ, afq, lo, aF, xH, hp, iV, eE, iK, bz, kn, y, ig, bvy, Pk, qR, hv); #c1(USO1) c2(NP_003706) c3 (general vesicular transport factor p115 isoform 2) c4(140291, 140292) c5(140293, 140294, 140295, 140296, 140297, 140298, 140299, 140300, 140301, 140302, 140303, 140304, 140305, 140306, 140307, 140308, 140309, 140310, 140311) c6( ) c7(oO, T, bvt, wL, if, jx, aA, eb, n, cl, fr, bvx, ci, qN, V, eW, o, ad, hX, jF, av, F, dY, h, K, lQ, hu, hH, bA, aa, ul, A, mR, dH, Y, KS, cr, eZ, afq, lo, aF, xH, hp, iV, eE, iK, bz, kn, y, ig, bvy, Pk, qR, hv); #c1(USD1) c2(XP_006714460) c3(general vesicular transport factor p115 isoform X2) c4(287844) c5( ) c6( ) c7(oO, T, bvt, wL, if, jx, aA, eb, n, cl, fr, bvx, ci, qN, V, eW, o, ad, hX, jF, av, F, dY, h, K, lQ, hu, hH, bA, aa, ul, A, mR, dH, Y, KS, cr, eZ, afq, lo, aF, xH, hp, iV, eE, iK, bz, kn, y, ig, bvy, Pk, qR, hv); #c1(USP10) c2(NP_005144) c3(ubiquitin carboxyl-terminal hydrolase 10 isoform 2) c4(153380) c5(153381, 153382, 153383, 153384, 153385, 153386, 153387) c6( ) c7(bB, lo, d, K); #c1(USP10) c2(XP_006721394) c3(ubiquitin carboxyl-terminal hydrolase 10 isoform X2) c4(293971, 293972) c5(293973, 293974, 293975, 293976, 293977, 293978) c6( ) c7(bB, lo, d, K); #c1(USP10) c2(XP_011521745) c3(ubiquitin carboxyl-terminal hydrolase 10 isoform X3) c4( ) c5(316451, 316452, 316453, 316454, 316455) c6( ) c7(bB, lo, d, K); #c1 (USP11) c2(NP_004642) c3(ubiquitin carboxyl-terminal hydrolase 11) c4(148459, 148460) c5(148461, 148462, 148463, 148464, 148465, 148466, 148467, 148468, 148469, 148470, 148471, 148472) c6( ) c7(os, lR, aE, oZ, kp, nA, od, nC); #c1(USP12) c2(NP_872294) c3(ubiquitin carboxyl-terminal hydrolase (2) c4(256030) c5(256031, 256032, 256033, 256034, 256035, 256036) c6( ) c7(IV); #c1(USP13) c2(NP_003931) c3(ubiquitin carboxyl-terminal hydrolase 13) c4(142268) c5(142269, 142270, 142271, 142272, 142273, 142274) c6( ) c7(aW, Pk, Y, lQ, aT); #c1(USP14) c2(NP_001032411) c3(ubiquitin carboxyl-terminal hydrolase 14 isoform b) c4(31321) c5(31322, 31323, 31324, 31325, 31326, 31327, 31328, 31329, 31330, 31331) c6( ) c7(T, fd, ci, aR, aJM, av, arA, Ak, ey, Kf, g, iV, as, cm, qs, bz, bF, ar, iS, azr, R, y); #c1(USP14) c2(NP_005142) c3(ubiquitin carboxyl-terminal hydrolase 14 isoform a) c4(153366, 153367) c5(153368, 153369, 153370, 153371, 153372, 153373, 153374, 153375, 153376, 153377, 153378, 153379) c6( ) c7(T, fd, ci, aR, aJM, av, arA, Ak, ey, Kf, g, iV, as, cm, qs, bz, bF, ar, iS, azr, R, y); #c1(USP15) c2(NP_001239007) c3 (ubiquitin carboxyl-terminal hydrolase 15 isoform 1) c4(88147) c5(88148, 88149, 88150, 88151, 88152, 88153) c6( ) c7(oO, T, d, aE, qY, fe, m, t, eA); #c1(USP15) c2(NP_001239008) c3(ubiquitin carboxyl-terminal hydrolase 15 isoform 3) c4(88154, 88155) c5(88156, 88157, 88158, 88159, 88160, 88161, 88162, 88163, 88164, 88165, 88166, 88167) c6( ) c7(oO, T, d, aE, qY, fe, m, t, eA); #c1(USP15) c2(NP_006304) c3(ubiquitin carboxyl-terminal hydrolase 15 isoform 2) c4(163857, 163858) c5(163859, 163860, 163861, 163862, 163863, 163864, 163865, 163866, 163867, 163868, 163869, 163870) c6( ) c7(oO, T, d, aE, qY, fe, m, t, eA); #c1(USP17L2) c2(NP_958804) c3(ubiquitin carboxyl-terminal hydrolase 17) c4(262208) c5(262209, 262210, 262211, 262212, 262213, 262214) c6( ) c7(T, K, eZ, aT); #c1(USP17L30) c2(NP_001243796) c3(ubiquitin specific peptidase 17-like family member 30) c4(91726) c5(91727, 91728, 91729, 91730, 91731, 91732, 91733, 91734, 91735, 91736, 91737, 91738, 91739, 91740, 91741, 91742, 91743, 91744, 91745, 91746, 91747, 91748, 91749, 91750) c6( ) c7(T, eZ); #c1(USP18) c2(NP_059110) c3(ubl carboxyl-terminal hydrolase 18) c4(193064, 193065) c5(193066, 193067, 193068, 193069, 193070, 193071) c6( ) c7(T, Bv, F, A, aZv, yG, jx, fc, lo, eo, K, cP, V, bz, er); #c1(USP18) c2(XP_006724137) c3(ubl carboxyl-terminal hydrolase 18 isoform X1) c4( ) c5(295811, 295812, 295813, 295814, 295815, 295816) c6( ) c7(T, Bv, F, A, aZv, yG, jx, fc, lo, eo, K, cP, V, bz, er); #c1(USP2) c2(NP_001230688) c3 (ubiquitin carboxyl-terminal hydrolase 2 isoform c) c4(86569) c5(86570, 86571, 86572, 86573, 86574, 86575) c6( ) c7(bL, T, B, ad, vH); #c1(USP2) c2(NP_741994) c3(ubiquitin carboxyl-terminal hydrolase 2 isoform b) c4(247538, 247539) c5(247540, 247541, 247542, 247543, 247544, 247545, 247546, 247547, 247548, 247549, 247550, 247551) c6( ) c7(bL, T, B, ad, vH); #c1(USP2) c2(XP_00527/779) c3(ubiquitin carboxyl-terminal hydrolase 2 isoform X1) c4(283584) c5(283585, 283586, 283587, 283588, 283589, 283590) c6( ) c7(bL, T, B, ad, vH); #c1(USP20) c2(XP_005251722) c3(ubiquitin carboxyl-terminal hydrolase 20 isoform X1) c4(271089) c5(271090, 271091, 271092, 271093, 271094, 271095, 271096, 271097, 271098, 271099, 271100, 271101, 271102, 271103) c6( ) c7(ac); #c1(USP22) c2(NP_056091) c3(ubiquitin carboxyl-terminal hydrolase 22) c4(185649, 185650, 185651) c5(185652, 185653, 185654, 185655, 185656, 185657, 185658, 185659, 185660, 185661, 185662, 185663, 185664, 185665, 185666, 185667, 185668, 185669) c6( ) c7(ck, T, nM, s, eM, bE, if, qK, r, bB, q, ci, qN, cd); #c1(USP25) c2(NP_001269970) c3(ubiquitin carboxyl-terminal hydrolase 25 isoform USP25m) c4(103310) c5(103311, 103312, 103313, 103314, 103315, 103316) c6( ) c7(fe, f, av, ww, eM); #c1(USP25) c2(NP_001269971) c3(ubiquitin carboxyl-terminal hydrolase 25 isoform USP25b) c4(103317) c5(103318, 103319, 103320, 103321, 103322, 103323) c6( ) c7(fe, f, av, ww, eM); #c1(USP25) c2(NP_037528) c3(ubiquitin carboxyl-terminal hydrolase 25 isoform USP25a) c4(176026) c5(176027, 176028, 176029, 176030, 176031, (76032) c6( ) c7(fe, f, av, ww, eM); #c1(USP26) c2(NP_114113) c3(ubiquitin carboxyl-terminal hydrolase 26) c4(222190) c5(222191, 222192, 222193, 222194, 222195, 222196, 222197, 222198, 222199, 222200) c6( ) c7(gU, ND, aMv, vH, UI, JO, UE); #c1(USP28) c2(NP_065937) c3(ubiquitin carboxyl-terminal hydrolase 28 isoform 1) c4(206331) c5(206332, 206333, 206334, 206335, 206336, 206337, 206338, 206339) c6( ) c7(T, aW, aT, r, n, dL, np, ci, KV, eL, q); #c1(USP28) c2(XP_005271696) c3(ubiquitin carboxyl-terminal hydrolase 28 isoform X10) c4(283534) c5(283535, 283536, 283537, 283538, 283539, 283540) c6( ) c7(T, aW, al, r, n, dL, np, ci, KV, eL, q); #c1(USP3) c2(NP_001243631) c3 (ubiquitin carboxyl-terminal hydrolase 3 isoform 2) c4(91259) c5(91260, 91261, 91262, 91263, 91264, 91265) c6( ) c7(cd); #c1(USP3) c2(NP_006528) c3(ubiquitin carboxyl-terminal hydrolase 3 isoform 1) c4(166009, 166010, 166011, 166012, 166013) c5(166014, 166015, 166016, 166017, 166018, 166019, 166020, 166021, 166022, 166023, 166024, 166025, 166026, 166027, 166028, 166029, 166030, 166031, 166032, 166033, 166034, 166035, 166036, 166037, 166038) c6( ) c7(cd); #c1(USP3) c2(XP_006720853) c3 (ubiquitin carboxyl-terminal hydrolase 3 isoform X2) c4( ) c5(293741, 293742, 293743, 293744, 293745) c6( ) c7(cd); #c1(USP3) c2(XP_011520568) c3(ubiquitin carboxyl-terminal hydrolase 3 isoform X5) c4(315492) c5(315493, 315494, 315495, 315496, 315497, 315498) c6( ) c7(cd); #c1(USP32) c2(NP_115971) c3(ubiquitin carboxyl-terminal hydrolase 32) c4(225000) c5(225001, 225002, 225003, 225004, 225005, 225006, 225007, 225008, 225009, 225010, 225011, 225012) c6( ) c7(bB, aW, lo, K, aT); #c1(USP32) c2(XP_011523675) c3(ubiquitin carboxyl-terminal hydrolase 32 isoform X3) c4(318165) c5( ) c6( ) c7(bB, aW, lo, K, aT); #c1(USP33) c2(XP_011539358) c3 (ubiquitin carboxyl-terminal hydrolase 33 isoform X4) c4(337440, 337441) c5(337442, 337443, 337444, 337445, 337446, 337447, 337448, 337449, 337450, 337451, 337452, 337453, 337454, 337455, 337456, 337457, 337458, 337459) c6( ) c7(x, k); #c1(USP36) c2(XP_005257599) c3(ubiquitin carboxyl-terminal hydrolase 36 isoform X1) c4(275274) c5(275275, 275276, 275277, 275278, 275279, 275280, 275281, 275282, 275283, 275284) c6( ) c7(t, m, an); #c1(USP36) c2(XP_011523369) c3(ubiquitin carboxyl-terminal hydrolase 36 isoform X2) c4(317893) c5( ) c6( ) c7(t, m, an); #c1(USP36) c2(XP_011523372) c3(ubiquitin carboxyl-terminal hydrolase 36 isoform X5) c4( ) c5(317894, 317895, 317896, 317897, 317898, 317899) c6( ) c7(t, m, an); #c1(USP36) c2(XP_011523373) c3(ubiquitin carboxyl-terminal hydrolase 36 isoform X6) c4(317900) c5( ) c6( ) c7(t, m, an); #c1(USP36) c2(XP_011523374) c3 (ubiquitin carboxyl-terminal hydrolase 36 isoform X7) c4( ) c5(317901, 317902, 317903, 317904, 317905, 317906) c6( ) c7(t, m, an); #c1(USP37) c2(XP_005246779) c3 (ubiquitin carboxyl-terminal hydrolase 37 isoform X1) c4(267414) c5(267415, 267416, 267417, 267418, 267419, 267420, 267421, 267422, 267423, 267424, 267425, 267426, 267427, 267428) c6( ) c7(jf, jj, ac); #c1(USP4) c2(NP_001238806) c3(ubiquitin carboxyl-terminal hydrolase 4 isoform c) c4(87908) c5(87909, 87910, 87911, 87912, 87913, 87914) c6( ) c7(T, ft, aW, aT, ci, ik, B); #c1(USP4) c2(NP_003354) c3(ubiquitin carboxyl-terminal hydrolase 4 isoform a) c4(137741) c5(137742, 137743, 137744, 137745, 137746, 137747) c6( ) c7(T, ft, aW, aT, ci, iK, B); #c1(USP4) c2(NP_955475) c3(ubiquitin carboxyl-terminal hydrolase 4 isoform b) c4(261842) c5(261843, 261844, 261845, 261846, 261847, 261848) c6( ) c7(T, ft, aW, aT, ci, iK, B); #c1(USP40) c2(NP_060688) c3(ubiquitin carboxyl-terminal hydrolase 40) c4(197165, 197166) c5(197167, 197168, 197169, 197170, 197171, 197172, 197173, 197174, 197175, 197176, 197177, 197178, 197179, 197180, 197181, 197182) c6( ) c7(GB, an); #c1(USP40) c2(XP_006712675) c3(ubiquitin carboxyl-terminal hydrolase 40 isoform X2) c4(286790) c5( ) c6( ) c7(GB, an); #c1(USP40) c2(XP_011509699) c3 (ubiquitin carboxyl-terminal hydrolase 40 isoform X3) c4(300272) c5( ) c6( ) c7(GB, an); #c1(USP40) c2(XP_011509703) c3(ubiquitin carboxyl-terminal hydrolase 40 isoform X7) c4( ) c5(300273, 300274, 300275, 300276, 300277, 300278) c6( ) c7(GB, an); #c1(USP40) c2(XP_011509704) c3(ubiquitin carboxyl-terminal hydrolase 40 isoform X8) c4( ) c5(300279, 300280, 300281, 300282, 300283, 300284) c6( ) c7(GB, an); #c1(USP42) c2(NP_115548) c3(ubiquitin carboxyl-terminal hydrolase 42) c4(223147) c5(223148, 223149, 223150, 223151, 223152, 223153) c6( ) c7(ck, T, bt); #c1(USP42) c2(XP_005249940) c3(ubiquitin carboxyl-terminal hydrolase 42 isoform X5) c4( ) c5(269943) c6( ) c7(ck, T, bt); #c1(USP43) c2(NP_001254505) c3(ubiquitin carboxyl-terminal hydrolase 43 isoform 2) c4(94576) c5(94577, 94578, 94579, 94580, 94581, 94582) c6( ) c7(fq, Ea); #c1(USP43) c2(NP_694942) c3(ubiquitin carboxyl-terminal hydrolase 43 isoform 1) c4(245453) c5(245454, 245455, 245456, 245457, 245458, 245459) c6( ) c7(fq, Ea); #c1(USP44) c2(NP_001265322) c3(ubiquitin carboxyl-terminal hydrolase 44) c4(98259) c5(98260, 98261, 98262, 98263, 98264, 98265, 98266, 98267, 98268, 98269, 98270, 98271, 98272, 98273) c6( ) c7(T, bz); #c1(USP44) c2(XP_006719691) c3(ubiquitin carboxyl-terminal hydrolase 44 isoform X3) c4(292883, 292884) c5(292885, 292886, 292887, 292888, 292889, 292890, 292891, 292892, 292893, 292894, 292895, 292896) c6( ) c7(T, bz); #c1(USP46) c2(NP_001127695) c3 (ubiquitin carboxyl-terminal hydrolase 46 isoform 2) c4(49231) c5(49232, 49233, 49234, 49235, 49236, 49237) c6( ) c7(aqv, bE, r, bB, bK, q); #c1(USP46) c2(NP_073743) c3(ubiquitin carboxyl-terminal hydrolase 46 isoform 1) c4(213426) c5(213427, 213428, 213429, 213430, 213431, 213432) c6( ) c7(aqv, bE, r, bB, bK, q); #c1(USP48) c2(XP_011540564) c3(ubiquitin carboxyl-terminal hydrolase 48 isoform X1) c4(339051) c5(339052, 339053, 339054, 339055, 339056, 339057) c6( ) c7(xa); #c1(USP49) c2(NP_001273483) c3(ubiquitin carboxyl-terminal hydrolase 49 isoform a) c4(104981, 104982) c5(104983, 104984, 104985, 104986, 104987, 104988, 104989, 104990, 104991, 104992, 104993, 104994, 104995, 104996, 104997, 104998, 104999, 105000, 105001, 105002) c6( ) c7(ac); #c1(USP49) c2(NP_061031) c3(ubiquitin carboxyl-terminal hydrolase 49 isoform b) c4(199083, 199084) c5(199085, 199086, 199087, 199088, 199089, 199090, 199091, 199092, 199093, 199094, 199095, 199096) c6( ) c7(ac); #c1(USP5) c2(NP_001092006) c3(ubiquitin carboxyl-terminal hydrolase 5 isoform 1) c4(39241) c5(39242, 39243, 39244, 39245, 39246, 39247) c6( ) c7(xZ, f, d, gu); #c1(USP5) c2(NP_003472) c3(ubiquitin carboxyl-terminal hydrolase 5 isoform 2) c4(138618) c5(138619, 138620, 138621, 138622, 138623, 138624) c6( ) c7(xZ, f, d, gu); #c1(USP6) c2(NP_001291213) c3(ubiquitin carboxyl-terminal hydrolase 6) c4(115584) c5(115585, 115586, 115587, 115588, 115589, 115590, 115591, 115592, 115593, 115594, 115595, 115596, 115597, 115598, 115599, 115600, 115601, 115602) c6( ) c7(T, acW, BL, aW, yg, al, n, azg, zu, asb); #c1(USP6) c2(XP_011522359) c3 (ubiquitin carboxyl-terminal hydrolase 6 isoform X2) c4(317030) c5( ) c6( ) c7(T, acW, BL, aW, yg, al, n, azg, zu, asb); #c1(USP6) c2(XP_011522360) c3 (ubiquitin carboxyl-terminal hydrolase 6 isoform X3) c4(317031, 317032) c5( ) c6( ) c7(T, acW, BL, aW, yg, aT, n, azg, zu, asb); #c1(USP6) c2(XP_011522361) c3(ubiquitin carboxyl-terminal hydrolase 6 isoform X4) c4(317033) c5(317034, 317035, 317036, 317037, 317038, 317039, 317040, 317041, 317042, 317043, 317044, 317045) c6( ) c7(T, acW, BL, aW, yg, al, n, azg, zu, asb); #c1(USP6NL) c2(NP_001073960) c3(USP6 N-terminal-like protein isoform 2) c4(36700) c5(36701, 36702, 36703, 36704, 36705, 36706) c6( ) c7(Y); #c1(USP6NL) c2(NP_055503) c3(USP6 N-terminal-like protein isoform 1) c4(181596) c5(181597, 181598, 181599, 181600, 181601, 181602, 181603, 181604, 181605, 181606) c6( ) c7(Y); #c1(USP7) c2(NP_001273386) c3(ubiquitin carboxyl-terminal hydrolase 7 isoform 2) c4(104818) c5(104819, 104820, 104821, 104822, 104823, 104824) c6( ) c7(T, aw, fd, bB, ci, qN, cd, ad, eW, cfd, aT, KP, K, aUm, nJ, ae, aW, ff, La, en, g, cm, ck, eM, bE, kS); #c1(USP7) c2(NP_003461) c3(ubiquitin carboxyl-terminal hydrolase 7 isoform 1) c4(138512, 138513, 138514) c5(138515, 138516, 138517, 138518, 138519, 138520, 138521, 138522, 138523, 138524, 138525, 138526, 138527, 138528, 138529, 138530, 138531, 138532) c6( ) c7(T, aw, fd, bB, ci, qN, cd, ad, eW, cfd, aT, KP, K, aUm, nJ, ae, aW, ff, La, en, g, cm, ck, eM, bE, kS); #c1(USP8) c2(NP_001122082) c3(ubiquitin carboxyl-terminal hydrolase 8 isoform a) c4(46402, 46403, 46404, 46405) c5(46406, 46407, 46408, 46409, 46410, 46411, 46412, 46413, 46414, 46415, 46416, 46417, 46418, 46419) c6( ) c7(OD, bz, d, OH, eM); #c1(USP8) c2(XP_006720825) c3 (ubiquitin carboxyl-terminal hydrolase 8 isoform X2) c4(293724) c5( ) c6( ) c7(OD, bz, d, OH, eM); #c1(USP8) c2(XP_011520496) c3(ubiquitin carboxyl-terminal hydrolase 8 isoform X3) c4( ) c5(315391, 315392, 315393, 315394, 315395, 315396, 315397, 315398, 315399, 315400, 315401, 315402, 315403, 315404, 315405, 315406, 315407, 315408, 315409, 315410, 315411, 315412, 315413, 315414) c6( ) c7(OD, bz, d, OH, eM); #c1(USP9X) c2(NP_001034679) c3(probable ubiquitin carboxyl-terminal hydrolase FAF-X isoform 3) c4(32069) c5(32070, 32071, 32072, 32073, 32074, 32075) c6( ) c7(T, cc, fM, aE, cTm, vJ, aW, iI, mx, H, aT, r, fd, kp, q, nA, dw, YZ, B); #c1(USP9X) c2(NP_001034680) c3(probable ubiquitin carboxyl-terminal hydrolase FAF-X isoform 4) c4(32076) c5(32077, 32078, 32079, 32080, 32081, 32082) c6( ) c7(T, cc, fM, aE, cTm, vJ, aW, iI, mx, H, aT, r, fd, kp, q, nA, dw, YZ, B); #c1(USP9Y) c2(XP_011529771) c3(probable ubiquitin carboxyl-terminal hydrolase FAF-Y isoform X1) c4(325441) c5(325442, 325443, 325444, 325445, 325446, 325447, 325448, 325449, 325450, 325451, 325452, 325453, 325454) c6( ) c7(vH, ND, gU, cyH, j); #c1(USP9Y) c2(XP_011529772) c3(probable ubiquitin carboxyl-terminal hydrolase FAF-Y isoform X2) c4(325455) c5( ) c6( ) c7(vH, ND, gU, cyH, j); #c1(USPL1) c2(NP_005791) c3(SUMO-specific isopeptidase USPL1) c4(159362) c5(159363, 159364, 159365, 159366, 159367, 159368) c6( ) c7(aW, aT); #c1(UST) c2(NP_005706) c3 (uronyl 2-sulfotransferase) c4(158666) c5(158667, 158668, 158669, 158670, 158671, 158672) c6( ) c7(dP, oe, j, aw); #c1(UTF1) c2(NP_003568) c3(undifferentiated embryonic cell transcription factor 1) c4(139226) c5(139227, 139228, 139229, 139230, 139231, 139232) c6( ) c7(vT, wu, if, eD, rO, ci, qN, wm, cm, KY); #c1(UTP14A) c2(NP_001159693) c3(U3 small nucleolar RNA-associated protein 14 homolog A isoform 2) c4(64601) c5(64602, 64603, 64604, 64605, 64606, 64607) c6( ) c7(K); #c1(UTP14A) c2(NP_006640) c3(U3 small nucleolar RNA-associated protein 14 homolog A isoform 1) c4(166834, 166835) c5(166836, 166837, 166838, 166839, 166840, 166841, 166842, 166843, 166844, 166845, 166846, 166847) c6( ) c7(K); #c1(UTP14A) c2(XP_011529566) c3(U3 small nucleolar RNA-associated protein 14 homolog A isoform X1) c4(325108, 325109) c5(325110, 325111, 325112, 325113, 325114, 325115, 325116, 325117, 325118, 325119, 325120, 325121) c6( ) c7(K); #c1(UTP14C) c2(NP_067677) c3(U3 small nucleolar RNA-associated protein 14 homolog C) c4(209282) c5(209283, 209284, 209285, 209286, 209287, 209288, 209289) c6( ) c7(gU); #c1(UTP20) c2(NP_055318) c3(small subunit processome component 20 homolog) c4(180416) c5(180417, 180418, 180419, 180420, 180421, 180422) c6( ) c7(dD, eq, bX, aT); #c1(UTRN) c2(NP_009055) c3(utrophin) c4(170475, 170476, 170477, 170478) c5(170479, 170480, 170481, 170482, 170483, 170484, 170485, 170486, 170487, 170488, 170489, 170490, 170491, 170492, 170493, 170494, 170495, 170496) c6( ) c7(ID, Ri, cz, KO, dG, c, Ae, oj, al, cy, bl, K, an, ly, hM, aW, xs, uY, t, lx, xA, wg, c, dc, bgo, U, N); #c(UTRN) c2(XP_005267184) c3(utrophin isoform X5) c4(280822) c5(280823, 280824, 280825, 280826, 280827, 280828) c6( ) c7(ID, Ri, cz, KO, dG, cc, Ae, oj, aT, cy, bl, K, an, ly, hM, aW, xs, uY, t, lx, xA, wg, c, dc, bgo, U, NN); #c1(UTRN) c2(XP_006715623) c3(utrophin isoform X10) c4(289647) c5(289648, 289649, 289650, 289651, 289652, 289653, 289654, 289655, 289656, 289657, 289658, 289659, 289660, 289661, 289662, 289663, 289664, 289665, 289666, 289667, 289668, 289669, 289670, 289671, 289672, 289673, 289674, 289675, 289676, 289677, 289678, 289679) c6( ) c7(ID, Ri, cz, KO, dG, cc, Ae, oj, aT, cy, bl, K, an, ly, hM, aW, xs, uY, t, lx, xA, wg, c, dc, bgo, U, NN); #c1(UTRN) c2(XP_011534407) c3 (utrophin isoform X4) c4(330887, 330888, 330889) c5(330890, 330891, 330892, 330893, 330894, 330895) c6( ) c7(ID, Ri, cz, KO, dG, cc, Ae, oj, aT, cy, bl, K, an, ly, hM, aW, xs, uY, t, lx, xA, wg, c, dc, bgo, U, NN); #c1(UTRN) c2(XP_011534411) c3 (utrophin isoform X9) c4(330896) c5(330897, 330898, 330899, 330900, 330901, 330902) c6( ) c7(ID, Ri, cz, KO, dG, cc, Ae, oj, aT, cy, bl, K, an, ly, hM, aW, xs, uY, t, lx, xA, wg, c, dc, bgo, U, NN); #c1(UTS2) c2(XP_011538839) c3(urotensin-2 isoform X1) c4(336859) c5(336860, 336861, 336862, 336863, 336864, 336865, 336866, 336867) c6(336868, 336869) c7(bL, bM, T, dD, eR, vf, ea, e, al, aw, cz, cR, bB, aj, v, j, eb, vp, eq, aY, cd, q, J, PC, dP, bv, Tg, cc, sN, Bc, aql, iv, cy, hH, Fg, s, cW, r, Hu, cm, uC, xw, pb, qa, kT, bE, mg, fP, bm, mf, dy, ei, lC, sC, y); #c1(UTS2) c2(XP_011538840) c3(urotensin-2 isoform X2) c4(336870, 336871, 336872) c5(336873, 336874, 336875, 336876, 336877, 336878, 336879, 336880, 336881, 336882, 336883, 336884, 336885, 336886, 336887, 336888, 336889, 336890, 336891, 336892, 336893, 336894, 336895, 336896) c6(336897, 336898, 336899, 336900, 336901, 336902) c7(bL, bM, T, dD, eR, vf, ea, e, al, aw, cz, cR, bB, aj, v, j, eb, vp, eq, aY, cd, q, J, PC, dP, bv, Tg, cc, sN, Bc, aql, iv, cy, hH, Fg, s, cW, r, Hu, cm, uC, xw, pb, qa, kT, bE, mg, fP, bm, mf, dy, ei, lC, sC, y); #c1(UTS2B) c2(NP_937795) c3(urotensin-28 precursor) c4(259097, 259098) c5(259099, 259100, 259101, 259102, 259103, 259104, 259105, 259106, 259107, 259108, 259109, 259110, 259111, 259112, 259113, 259114, 259115, 259116, 259117, 259118, 259119) c6(259120, 259121, 259122, 259123, 259124) c7(dD, aw, uG, mg, jj, eq, lC, af); #c1(UTS2R) c2(NP_061822) c3(urotensin-2 receptor) c4(200519) c5(200520, 200521, 200522, 200523, 200524, 200525) c6( ) c7(bM, T, aC, pf, ea, if, e, boZ, CH, bB, fd, cL, HH, aBm, aY, W, Tz, aR, x, bv, AW, aji, u, Bc, M, dV, K, qM, bU, hH, eL, eN, hm, li, eG, yo, bbC, dH, aW, aAu, ih, alR, hl, apn, Ku, Eg, g, eE, eF, cm, mZ, wT, eY, y, jV, eM, iA, wK, dc, blQ, vA, ZT, bxS, U, cj, vy, Gs, uJ, azg, k, m, R, pw, bi, gD, iu, dD, gU, aEj, RX, aw, aj, ww, afE, fr, nA, pM, qN, eW, axC, biS, cc, no, ec, BR, bKk, Nn, gd, Ak, h, dL, ey, ZW, mV, btr, mY, db, on, pl, he, vx, mU, A, mR, zb, fe, aq, op, mX, W, ky, cEz, dB, Fi, anZ, eD, yP, Ei, hL, bm, E, fL, bD, jd, it, mT, B, cn, qc, bL, aoU, fM, eR, bcb, jx, aA, cR, mS, ti, cz, j, v, C, Wa, vp, q, J, o, eA, cF, RI, mP, WY, jZ, aql, MI, aT, vH, qv, bK, zp, bq, aUQ, XZ, an, alV, ae, af, rE, jf, qw, s, iU, Y, yL, r, aaE, lo, aDm, HL, bwL, cl, t, iK, bN, ck, Qi, rR, nM, ac, c, bgy, ig, MB, AH, ro, dy, mf, aan, byK, gT, lJ, ao, gV, bNo, aDy, qb, aZ, aD, ha, iW, mw, bP, bS, OH, jE, uu, eq, Fb, mC, ad, Xv, dP, hX, HF, l, F, hd, Rq, akR, CJ, mW, ds, nI, oW, LA, hD, vc, hP, lQ, ji, yh, yF, mu, ael, d, lR, tb, hG, ctG, Gv, wc, bpO, nh, H, dN, gl, kD, yy, aF, eZ, Pd, iR, iV, aUC, bz, dr, qa, dR, eK, es, bF, pc, csw, bE, z, anY, afd, be, ei, hE, aAN, X, us); #c1(UTY) c2(NP_001245181) c3(histone demethylase UTY isoform 7) c4(92633) c5( ) c6( ) c7(j); #c1(UTY) c2(NP_001245183) c3(histone demethylase UTY isoform 9) c4(92634) c5(92635, 92636, 92637, 92638, 92639, 92640) c6( ) c7(j); #c1(UTY) c2(NP_001245189) c3(histone demethylase UTY isoform 15) c4(92641) c5( ) c6( ) c7(j); #c1(UTY) c2(NP_001245192) c3(histone demethylase UTY isoform 18) c4(92642) c5( ) c6( ) c7(j); #c1(UTY) c2(NP_009056) c3(histone demethylase UTY isoform 3) c4(170497) c5( ) c6( ) c7(j); #c1(UTY) c2(NP_872601) c3(histone demethylase UTY isoform 1) c4(256468, 256469) c5(256470, 256471, 256472, 256473, 256474, 256475, 256476, 256477, 256478, 256479, 256480, 256481, 256482, 256483, 256484, 256485, 256486, 256487, 256488, 256489) c6( ) c7(j); #c1(UTY) c2(XP_011529751) c3(histone demethylase UTY isoform X9) c4( ) c5(325397, 325398, 325399, 325400, 325401, 325402) c6( ) c7(j); #c1(UTY) c2(XP_011529752) c3(histone demethylase UTY isoform X10) c4( ) c5(325403, 325404, 325405, 325406, 325407, 325408) c6( ) c7(j); #c1(UTY) c2(XP_011529754) c3(histone demethylase UTY isoform X12) c4( ) c5(325409, 325410, 325411, 325412, 325413, 325414, 325415, 325416, 325417, 325418, 325419, 325420) c6( ) c7(j); #c1(UTY) c2(XP_011529761) c3(histone demethylase UTY isoform X17) c4(325421) c5( ) c6( ) c7(j); #c1(UTY) c2(XP_011529762) c3(histone demethylase UTY isoform X19) c4( ) c5(325422) c6( ) c7(j); #c1(UTY) c2(XP_011529763) c3(histone demethylase UTY isoform X21) c4(325423) c5( ) c6( ) c7(j); #c1(UTY) c2(XP_011529764) c3(histone demethylase UTY isoform X22) c4(325424, 325425, 325426) c5(325427, 325428, 325429, 325430, 325431, 325432) c6( ) c7(j); #c1(UTY) c2(XP_011529767) c3(histone demethylase UTY isoform X26) c4(325433) c5( ) c6( ) c7(j); #c1(UVRAG) c2(NP_003360) c3(UV radiation resistance-associated gene protein) c4(137770, 137771) c5(137772, 137773, 137774, 137775, 137776, 137777, 137778, 137779, 137780, 137781, 137782, 137783, 137784, 137785, 137786, 137787, 137788, 137789) c6( ) c7(aDR, bM, T, Fv, if, bB, qj, ci, Na, aOS, aE, arA, pX, K, hu, eL, li, aW, aEI, az, Az, aOW, OZ, LZ, ark, g, eF, eM, nB, aPc, m, iB, zt, ht, n, qN, aPb, ft, amJ, dL, Dz, aOU, aOX, bmc, aiB, MD, ks, aOT, fM, yk, aA, arN, AO, akG, q, er, aDY, qK, aT, rO, Nc, Tj, avt, aOV, ae, xR, Lf, Y, r, en, bu, t, iK, sS, QG, kP, zu, rA, zU, ad, aOZ, hd, Db, lQ, ff, eH, cr, asV, Ej, af, bz, atb, dr, LB, bE, adg, aPa); #c1(UVRAG) c2(XP_011543528) c3(UV radiation resistance-associated gene protein isoform X1) c4(342447) c5( ) c6( ) c7(aOR, bM, T, Fv, if, bB, qj, ci, Na, aOS, aE, arA, pX, K, hu, eL, li, aW, aEI, az, Az, aOW, OZ, LZ, ark, g, eF, eM, nB, aPc, m, iB, zt, ht, n, qN, aPb, ft, amJ, dL, Dz, aOU, aOX, bmc, aiB, MD, ks, aOT, fM, yk, aA, arN, AO, akG, q, er, aDY, qK, aT, rO, Nc, Tj, avt, aOV, ae, xR, Lf, Y, r, en, bu, t, iK, sS, QG, kP, zu, rA, zU, ad, aDZ, hd, Db, lQ, ff, eH, cr, asV, Ej, aF, bz, atb, dr, LB, bE, adg, aPa); #c1(UVSSA) c2(XP_011511824) c3(UV-stimulated scaffold protein A isoform X5) c4( ) c5(302496, 302497, 302498, 302499) c6( ) c7(cTn, KP, La, bnH); #c1(UVSSA) c2(XP_011511832) c3(UV-stimulated scaffold protein A isoform X7) c4(302500) c5( ) c6( ) c7(cTn, KP, La, bnH); #c1(UVSSA) c2(XP_011511836) c3(UV-stimulated scaffold protein A isoform X11) c4( ) c5(302501, 302502, 302503, 302504, 302505, 302506, 302507, 302508, 302509) c6( ) c7(cTn, KP, La, bnH); #c1(UXS1) c2(NP_001240804) c3(UDP-glucuronic acid decarboxylase 1 isoform 1) c4(88835, 88836, 88837, 88838, 88839, 88840) c5(88841, 88842, 88843, 88844, 88845, 88846) c6( ) c7(ac, bpc, Az); #c1(UXS1) c2(NP_001240805) c3(UDP-glucuronic acid decarboxylase 1 isoform 3) c4( ) c5(88847, 88848, 88849, 88850, 88851, 88852, 88853, 88854, 88855, 88856, 88857, 88858, 88859, 88860, 88861, 88862, 88863, 88864, 88865, 88866, 88867, 88868, 88869, 88870, 88871, 88872) c6( ) c7(ac, bpc, Az); #c1(UXS1) c2(NP_079352) c3(UDP-glucuronic acid decarboxylase 1 isoform 2) c4(217812, 217813) c5(217814, 217815, 217816, 217817, 217818, 217819) c6( ) c7(ac, bpc, Az); #c1(UXS1) c2(XP_006712838) c3(UDP-glucuronic acid decarboxylase 1 isoform X4) c4( ) c5(286891, 286892, 286893) c6( ) c7(ac, bpc, Az); #c1(UXS1) c2(XP_011510205) c3(UDP-glucuronic acid decarboxylase 1 isoform X2) c4( ) c5(300783, 300784, 300785, 300786, 300787, 300788) c6( ) c7(ac, bpc, Az); #c1(UXS1) c2(XP_011510206) c3(UDP-glucuronic acid decarboxylase 1 isoform X3) c4( ) c5(300789) c6( ) c7(ac, bpc, Az); #c1(UXT) c2(NP_004173) c3(protein UXT isoform 2) c4(144080, 144081) c5(144082, 144083, 144084, 144085, 144086, 144087, 144088, 144089, 144090, 144091) c6( ) c7(T, ae, cl, ad); #c1(UXT) c2(NP_705582) c3(protein UXT isoform 1) c4(246117) c5(246118, 246119, 246120, 246121, 246122, 246123, 246124, 246125) c6( ) c7(T, ae, cl, ad); #c1(VAC14) c2(NP_060522) c3(protein VAC14 homolog) c4(196356, 196357) c5(196358, 196359, 196360, 196361, 196362, 196363, 196364, 196365, 196366, 196367, 196368, 196369) c6( ) c7(h, B, bU, kH); #c1(VAMP1) c2(NP_001284367) c3(vesicle-associated membrane protein 1 isoform 4) c4( ) c5(111838, 111839, 111840, 111841, 111842, 111843, 111844) c6( ) c7(YE, aR, s, Gx); #c1(VAMP1) c2(NP_055046) c3(vesicle-associated membrane protein 1 isoform 1) c4(178558, 178559, (78560) c5(178561, 178562, 178563, 178564, 178565, 178566, 178567, 178568, 178569, 178570, 178571, 178572) c6( ) c7(YE, aR, s, Gx); #c1(VAMP1) c2(NP_058439) c3(vesicle-associated membrane protein 1 isoform 3) c4(192744) c5(192745, 192746, 192747, 192748, 192749) c6( ) c7(YE, aR, s, Gx); #c1(VAMP1) c2(NP_954740) c3(vesicle-associated membrane protein 1 isoform 2) c4(261361) c5(261362, 261363, 261364, 261365, 261366, 261367) c6( ) c7(YE, aR, s, Gx); #c1(VAMP2) c2(NP_055047) c3(vesicle-associated membrane protein 2) c4(178573) c5(178574, 178575, 178576, 178577, 178578, 178579) c6( ) c7(bL, T, ha, Gx, bK, nA, kk, af); #c1(VAMP4) c2(NP_001172056) c3(vesicle-associated membrane protein 4 isoform 2) c4(72363) c5(72364, 72365, 72366, 72367, 72368, 72369, 72370, 72371, 72372, 72373) c6( ) c7(bL, aj, J); #c1(VAMP4) c2(NP_003753) c3(vesicle-associated membrane protein 4 isoform 1) c4(140631) c5(140632, 140633, 140634, 140635, 140636, 140637, 140638, 140639, 140640, 140641) c6( ) c7(bL, aj, J); #c1(VAMP7) c2(NP_001138621) c3(vesicle-associated membrane protein 7 isoform 2) c4(57247) c5(57248, 57249, 57250, 57251, 57252, 57253) c6( ) c7(mU, T, cA, mW, aW, mZ, aT, mS, mY, bK, eA, kk, mV, iK, mT, Zj, mX); #c1(VAMP7) c2(NP_001172112) c3(vesicle-associated membrane protein 7 isoform 3) c4(72408) c5(72409, 72410, 72411, 72412, 72413, 72414, 72415) c6( ) c7(mU, T, cA, mW, aW, mZ, aT, mS, mY, bK, eA, kk, mV, iK, mT, Zj, mX); #c1(VAMP7) c2(NP_005629) c3(vesicle-associated membrane protein 7 isoform 1) c4(158090) c5(158091, 158092, 158093, 158094, 158095) c6( ) c7(mU, T, cA, mW, aW, mZ, aT, mS, mY, bK, eA, kk, mV, iK, mT, Zj, mX); #c1(VAMP8) c2(NP_003752) c3(vesicle-associated membrane protein 8) c4(140624) c5(140625, 140626, 140627, 140628, 140629, 140630) c6( ) c7(T, dD, ea, e, aw, j, ci, eq, fr, zP, jy, ey, vx, eu, zQ, oV, eH, eF, ac, iA, gZ, dy); #c1(VANGL2) c2(NP_065068) c3(vang-like protein 2) c4(203578) c5(203579, 203580, 203581, 203582, 203583, 203584) c6( ) c7(md, T, iU, lp, Y, r, aA, aF, oQ, Bd, q); #c1(VAPA) c2(NP_003565) c3(vesicle-associated membrane protein-associated protein A isoform 1) c4(139204) c5(139205, 139206, 139207, 139208, 139209, 139210) c6( ) c7(ac, c, uJ, bK, j, yA, fq); #c1(VAPA) c2(NP_919415) c3(vesicle-associated membrane protein-associated protein A isoform 2) c4(258546) c5(258547, 258548, 258549, 258550, 258551, 258552) c6( ) c7(ac, c, uJ, bK, j, yA, fq); #c1(VAPB) c2(NP_001182606) c3(vesicle-associated membrane protein-associated protein B/C isoform 2) c4(75108) c5(75109, 75110, 75111, 75112, 75113, 75114) c6( ) c7(cTo, c, Ol, HC, aT, kf, U, PJ, cTp, cTq); #c1(VAPB) c2(NP_004729) c3(vesicle-associated membrane protein-associated protein B/C isoform 1) c4(149123) c5(149124, 149125, 149126, 149127, 149128, 149129) c6( ) c7(cTo, c, DI, HC, aT, kf, U, PJ, cTp, cTq); #c1(VARS) c2(NP_006286) c3(valine-tRNA ligase) c4(163600, 163601, 163602, 163603) c5(163604, 163605, 163606, 163607, 163608, 163609, 163610, 163611, 163612, 163613, 163614, 163615, 163616, 163617, 163618, 163619, 163620, 163621, 163622, 163623, 163624, 163625, 163626, 163627, 163628, 163629, 163630, 163631, 163632, 163633, 163634, 163635, 163636, 163637, 163638, 163639, 163640, 163641, 163642, 163643, 163644, 163645, 163646, 163647, 163648, 163649, 163650, 163651, 163652, 163653, 163654, 163655, 163656, 163657, 163658, 163659, 163660, 163661, 163662, 163663, 163664, 163665, 163666, 163667, 163668, 163669, 163670, 163671, 163672, 163673, 163674, 163675, 163676, 163677, 163678, 163679, 163680, 163681, 163682, 163683, 163684) c6( ) c7(kC, eZ); #c1(VARS) c2(XP_005249419) c3(valine-tRNA ligase isoform X1) c4(269304) c5( ) c6( ) c7(kC, eZ); #c1(VARS) c2(XP_005274943) c3(valine-tRNA ligase isoform X1) c4(285541, 285542, 285543) c5( ) c6( ) c7(kC, eZ); #c1(VARS) c2(XP_011513147) c3(valine-tRNA ligase isoform X2) c4(304975, 304976) c5(304977, 304978, 304979, 304980, 304981, 304982, 304983, 304984, 304985, 304986, 304987, 304988, 304989, 304990, 304991, 304992, 304993, 304994, 304995, 304996, 304997, 304998, 304999, 305000, 305001, 305002, 305003, 305004, 305005, 305006, 305007, 305008, 305009, 305010, 305011, 305012) c6( ) c7(kC, eZ); #c1(VARS2) c2(NP_001161205) c3(valine-tRNA ligase, mitochondrial isoform 3) c4(65390) c5( ) c6( ) c7(hH, kC, AW, eZ); #c1(VARS2) c2(NP_001161206) c3(valine-tRNA ligase, mitochondrial isoform 1) c4(65391, 65392, 65393, 65394) c5(65395, 65396, 65397, 65398, 65399, 65400, 65401, 65402, 65403, 65404, 65405, 65406, 65407, 65408, 65409, 65410, 65411, 65412, 65413, 65414, 65415, 65416, 65417, 65418, 65419, 65420, 65421, 65422, 65423, 65424, 65425, 65426, 65427, 65428, 65429, 65430, 65431, 65432, 65433, 65434, 65435, 65436, 65437, 65438, 65439, 65440, 65441, 65442, 65443, 65444, 65445, 65446, 65447, 65448, 65449, 65450, 65451, 65452, 65453, 65454, 65455, 65456, 65457, 65458, 65459, 65460, 65461, 65462, 65463, 65464, 65465, 65466, 65467, 65468, 65469, 65470, 65471, 65472, 65473, 65474, 65475, 65476, 65477, 65478, 65479, 65480, 65481, 65482, 65483, 65484, 65485, 65486, 65487, 65488, 65489, 65490, 65491, 65492, 65493, 65494, 65495) c6( ) c7(hH, kC, AW, eZ); #c1(VARS2) c2(NP_065175) c3(valine-tRNA ligase, mitochondrial isoform 2 precursor) c4(204392, 204393, 204394, 204395, 204396, 204397, 204398, 204399, 204400, 204401) c5(204402, 204403, 204404, 204405, 204406, 204407, 204408, 204409, 204410, 204411, 204412, 204413, 204414, 204415, 204416, 204417, 204418, 204419, 204420, 204421, 204422, 204423, 204424, 204425, 204426, 204427, 204428, 204429, 204430, 204431, 204432, 204433, 204434, 204435, 204436, 204437, 204438, 204439, 204440, 204441, 204442, 204443, 204444, 204445, 204446, 204447, 204448, 204449, 204450, 204451, 204452, 204453, 204454, 204455, 204456, 204457, 204458, 204459, 204460, 204461) c6( ) c7(hH, kC, AW, eZ); #c1(VASH1) c2(NP_055724) c3(vasohibin-1) c4(183118, 183119) c5(183120, 183121, 183122, 183123, 183124, 183125, 183126, 183127) c6(183128, 183129) c7(bL, gk, DA, T, aE, qt, uG, al, aT, r, aj, v, j, K, ci, mo, eS, q, J); #c1(VASH1) c2(XP_006720143) c3(vasohibin-1 isoform X3) c4( ) c5(293284, 293285, 293286, 293287, 293288, 293289, 293290, 293291) c6(293292, 293293) c7(bL, gk, DA, T, aE, qt, uG, al, aT, r, aj, v, j, K, ci, mo, eS, q, J); #c1(VASH2) c2(NP_001129946) c3(vasohibin-2 isoform 2) c4(52334) c5(52335, 52336, 52337, 52338, 52339, 52340, 52341, 52342) c6( ) c7(T, aq, m, ir, iR, t, B); #c1(VASH2) c2(NP_001129947) c3(vasohibin-2 isoform 3) c4( ) c5(52343, 52344, 52345, 52346, 52347) c6( ) c7(T, aq, m, ir, iR, t, B); #c1(VASH2) c2(NP_001287985) c3(vasohibin-2 isoform 4) c4(113294, 113295) c5(113296, 113297, 113298, 113299, 113300, 113301, 113302, 113303) c6( ) c7(T, aq, m, ir, iR, t, B); #c1(VASH2) c2(NP_079025) c3(vasohibin-2 isoform 1) c4(216639) c5(216640, 216641, 216642, 216643, 216644, 216645) c6( ) c7(T, aq, m, ir, iR, t, B); #c1(VASH2) c2(XP_011508292) c3(vasohibin-2 isoform X4) c4( ) c5(298981) c6( ) c7(T, aq, m, ir, iR, t, B); #c1(VASH2) c2(XP_011508293) c3(vasohibin-2 isoform X5) c4(298982, 298983) c5(298984) c6( ) c7(T, aq, m, ir, iR, t, B); #c1(VASH2) c2(XP_011508297) c3(vasohibin-2 isoform X7) c4( ) c5(298985, 298986, 298987, 298988, 298989) c6( ) c7(T, aq, m, ir, iR, t, B); #c1(VASH2) c2(XP_011508299) c3(vasohibin-2 isoform X9) c4( ) c5(298990) c6( ) c7(T, aq, m, ir, iR, t, B); #c1(VASH2) c2(XP_011508300) c3(vasohibin-2 isoform X10) c4( ) c5(298991, 298992, 298993, 298994, 298995, 298996) c6( ) c7(T, aq, m, ir, iR, t, B); #c1(VASP) c2(NP_003361) c3(vasodilator-stimulated phosphoprotein) c4(137790) c5( ) c6( ) c7(T, aW, e, dh, az, mx, aT, aA, By, dG, aF, K, Sm); #c1(VASP) c2(XP_005259257) c3 (vasodilator-stimulated phosphoprotein isoform X2) c4( ) c5(276015, 276016, 276017, 276018, 276019, 276020, 276021) c6( ) c7(T, aW, e, dh, az, mx, aT, aA, By, dG, aF, K, Sm); #c1(VAT1) c2(NP_006364) c3(synaptic vesicle membrane protein VAT-1 homolog) c4(164381, 164382, 164383, 164384) c5(164385, 164386, 164387, 164388, 164389, 164390, 164391, 164392, 164393, 164394, 164395, 164396, 164397, 164398, 164399, 164400, 164401, 164402, 164403, 164404, 164405, 164406, 164407, 164408, 164409) c6( ) c7(aW, d, eA, eP, aT); #c1(VAT1L) c2(NP_065978) c3(synaptic vesicle membrane protein VAT-1 homolog-like) c4(206584) c5(206585, 206586, 206587, 206588, 206589, 206590) c6( ) c7(he, ac); #c1(VAV1) c2(NP_001245136) c3(proto-oncogene vav isoform 3) c4(92553, 92554) c5(92555, 92556, 92557, 92558, 92559, 92560, 92561, 92562, 92563, 92564, 92565, 92566) c6( ) c7(T, aC, fM, dA, fZ, aA, dP, aE, aT, K, jj, pk, jf, VC, A, aiB, et, iE, aW, Y, CM, kp, aF, g, cm, bz, Wt, ck, cj, k, bt, B); #c1(VAV1) c2(NP_005419) c3(proto-oncogene vav isoform 1) c4(155874) c5(155875, 155876, 155877, 155878, 155879, 155880, 155881, 155882, 155883, 155884) c6( ) c7(T, aC, fM, dA, fZ, aA, dP, aE, aT, K, jj, pk, jf, VC, A, aiB, et, iE, aW, Y, CM, kp, aF, g, cm, bz, Wt, ck, cj, k, bt, B); #c1(VAV2) c2(NP_001127870) c3(guanine nucleotide exchange factor VAV2 isoform 1) c4(49411) c5(49412, 49413, 49414, 49415, 49416, 49417) c6( ) c7(pZ, dH, aW, ff, sa, aT, By, K, en, t, V); #c1(VAV2) c2(NP_003362) c3(guanine nucleotide exchange factor VAV2 isoform 2) c4(137791) c5(137792, 137793, 137794, 137795, 137796, 137797) c6( ) c7(pZ, dH, aW, ff, sa, aT, By, K, en, t, V); #c1(VAV2) c2(XP_005272270) c3(guanine nucleotide exchange factor VAV2 isoform X2) c4(283937, 283938) c5(283939, 283940, 283941, 283942, 283943, 283944, 283945, 283946, 283947, 283948, 283949, 283950) c6( ) c7(pZ, dH, aW, ff, sa, aT, By, K, en, t, V); #c1(VAV3) c2(XP_011538805) c3(guanine nucleotide exchange factor VAV3 isoform X2) c4(336544) c5( ) c6( ) c7(ae, T, fM, d, pZ, dH, aW, aT, aA, n, cz, aF, ey, dw, gT, sa, fs, ad); #c1(VAX1) c2(NP_001106175) c3(ventral anterior homeobox 1 isoform a) c4(42322) c5(42323, 42324, 42325, 42326, 42327, 42328) c6( ) c7(apM, kj, ff, cTr, n, dL, en, g, eL, bvU); #c1(VAX1) c2(NP_954582) c3(ventral anterior homeobox 1 isoform b) c4(261058) c5(261059, 261060, 261061, 261062, 261063, 261064) c6( ) c7(apM, kj, ff, cTr, n, dL, en, g, eL, bvU); #c1(VAX2) c2(NP_036608) c3(ventral anterior homeobox 2) c4(174794) c5(174795, 174796, 174797, 174798, 174799, 174800, 174801) c6( ) c7(tT); #c1(VBP1) c2(NP_001290472) c3(prefoldin subunit 3 isoform 2) c4(115504) c5(115505) c6( ) c7(h, dz, fr, VB, py); #c1(VBP1) c2(NP_001290473) c3(prefoldin subunit 3 isoform 3) c4(115506) c5(115507, 115508, 115509, 115510, 115511, 115512) c6( ) c7(h, dz, fr, VB, py); #c1(VBP1) c2(NP_003363) c3(prefoldin subunit 3 isoform 1) c4( ) c5(137798, 137799, 137800, 137801, 137802) c6( ) c7(h, dz, fr, VB, py); #c1(VCAN) c2(NP_001119808) c3(versican core protein isoform 2 precursor) c4(44297) c5(44298, 44299, 44300, 44301, 44302, 44303) c6( ) c7(rt, G, sY, T, pG, kh, dD, bM, ea, yk, hV, if, hE, aw, aA, fd, Fb, eq, ci, rm, qN, fr, lu, q, ad, eA, hX, dP, fU, ip, ld, qK, aT, lG, dL, K, fb, ae, eL, bf, Ln, aW, Y, eH, r, aq, aF, as, cl, pL, aRq, cM, t, uC, op, eF, iR, iV, kT, acW, dB, hk, vb, vA, hL, bm, aS, m, dy, fy, B, us); #c1(VCAN) c2(NP_001157569) c3(versican core protein isoform 3 precursor) c4(62246) c5(62247, 62248, 62249, 62250, 62251, 62252) c6( ) c7(rt, G, sY, T, pG, kh, dD, bM, ea, yk, hV, if, hE, aw, aA, fd, Fb, eq, ci, rm, qN, fr, lu, q, ad, eA, hX, dP, fU, ip, ld, qK, aT, lG, dL, K, fb, ae, eL, bf, Ln, aW, Y, eH, r, aq, af, as, cl, pL, aRq, cM, t, uC, op, eF, iR, iV, kT, acW, dB, hk, vb, vA, hL, bm, aS, m, dy, fy, B, us); #c1(VCAN) c2(NP_001157570) c3(versican core protein isoform 4 precursor) c4(62253, 62254) c5(62255, 62256, 62257, 62258, 62259, 62260, 62261, 62262, 62263, 62264, 62265, 62266) c6( ) c7(rt, G, sY, T, pG, kh, dD, bM, ea, yk, hV, if, hE, aw, aA, fd, Fb, eq, ci, rm, qN, fr, lu, q, ad, eA, hX, dP, fU, ip, ld, qK, aT, lG, dL, K, fb, ae, eL, bf, Ln, aW, Y, eH, r, aq, af, as, cl, pL, aRq, cM, t, uC, op, eF, iR, iV, kT, acW, dB, hk, vb, vA, hL, bm, aS, m, dy, fy, B, us); #c1(VCAN) c2(NP_004376) c3(versican core protein isoform 1 precursor) c4(145903) c5(145904, 145905, 145906, 145907, 145908, 145909) c6( ) c7(rt, G, sY, T, pG, kh, dD, bM, ea, yk, hV, if, hE, aw, aA, fd, Fb, eq, ci, rm, qN, fr, lu, q, ad, eA, hX, dP, fU, ip, ld, qK, aT, lG, dL, K, fb, ae, eL, bf, Ln, aW, Y, eH, r, aq, af, as, cl, pL, aRq, cM, t, uC, op, eF, iR, iV, kT, acW, dB, hk, vb, vA, hL, bm, aS, m, dy, fy, B, us); #c1(VCL) c2(NP_003364) c3(vinculin isoform VCL) c4(137803, 137804) c5(137805, 137806, 137807, 137808, 137809, 137810, 137811, 137812, 137813, 137814, 137815, 137816, 137817, 137818, 137819, 137820, 137821, 137822) c6( ) c7(T, dD, ea, cTt, ci, BF, eq, fr, eA, ad, av, cc, hw, pX, cTs, By, vc, K, aGh, ae, bA, xs, bz, Qx, nG, mx); #c1(VCL) c2(NP_054706) c3(vinculin isoform meta-VCL) c4(177328, 177329) c5(177330, 177331, 177332, 177333, 17334, 17335, 177336, 177337, 177338, 177339, 177340, 177341) c6( ) c7(T, dD, ea, cTt, ci, BF, eq, fr, eA, ad, av, cc, hw, pX, cTs, By, vc, K, aGh, ae, bA, xs, bz, Qx, nG, mx); #c1(VCL) c2(XP_005270199) c3(vinculin isoform X1) c4(283263) c5(283264, 283265, 283266, 283267, 283268, 283269) c6( ) c7(T, dD, ea, cOt, ci, BF, eq, fr, eA, ad, av, cc, hw, pX, cTs, By, vc, K, aGh, ae, bA, xs, bz, Qx, nG, mx); #c1(VCL) c2(XP_005270200) c3 (vinculin isoform X2) c4(283270) c5( ) c6( ) c7(T, dD, ea, cTt, ci, BF, eq, fr, eA, ad, av, cc, hw, pX, cTs, By, vc, K, aGh, ae, bA, xs, bz, Qx, nG, mx); #c1(VCP) c2(NP_009057) c3(transitional endoplasmic reticulum ATPase) c4(170498) c5(170499, 170500, 170501, 170502, 170503, 170504) c6( ) c7(T, uf, alW, Ol, if, it, n, Vd, ci, qN, PJ, aR, xt, ad, cc, no, aE, aV, Do, zw, WY, Ws, Dq, oj, aT, Ak, aJI, aiW, cy, bdM, K, bl, hu, bV, an, chD, ae, af, aW, bht, Y, Lf, alR, aaE, bOW, aF, bu, eF, alj, dr, ck, rR, bb, Ew, c, Kv, eM, bEf, bxS, U, cTu, axV, oh, nw, m, fD, bi, lM, xg, B, ao); #c1(VCX) c2(NP_038480) c3(variable charge X-linked protein 1) c4(176710, 176711) c5(176712, 176713) c6( ) c7(btu, oe, nA, hQ); #c1(VCX) c2(XP_011543792) c3(variable charge X-linked protein 1 isoform X1) c4(342908) c5( ) c6( ) c7(btu, oe, nA, hQ); #c1(VCX2) c2(NP_057462) c3(variable charge X-linked protein 2) c4(191061, 191062) c5(191063, 191064, 191065, 191066, 191067, 191068, 191069, 191070, 191071, 191072, 191073, 191074, 191075, 191076, 191077, 191078, 191079, 191080, 191081, 191082, 191083, 191084, 191085, 191086, 191087, 191088, 191089, 191090, 191091, 191092, 191093, 191094, 191095, 191096) c6( ) c7(btu, nA, hQ); #c1(VCX3A) c2(NP_057463) c3(variable charge X-linked protein 3) c4(191097, 191098, 191099) c5( ) c6( ) c7(nf, btu, hQ, nA, Kn); #c1(VCX3A) c2(XP_006724557) c3(variable charge X-linked protein 3 isoform X1) c4(296076) c5( ) c6( ) c7(nf, btu, hQ, nA, Kn); #c1(VCX3B) c2(NP_001001888) c3(variable charge X-linked protein 3B) c4(19068, 19069) c5(19070, 19071, 19072, 19073, 19074, 19075, 19076, 19077, 19078, 19079, 19080, 19081, 19082, 19083, 19084, 19085, 19086, 19087, 19088, 19089, 19090, 19091, 19092) c6( ) c7(btu, nA, hQ); #c1(VCY) c2(NP_004670) c3(testis-specific basic protein Y 1) c4(148713) c5(148714, 148715, 148716, 148717, 148718, 148719, 148720, 148721, 148722) c6( ) c7(btu, nA, hQ); #c1(VDAC1) c2(XP_005272132) c3(voltage-dependent anion-selective channel protein 1 isoform X1) c4(283715) c5(283716, 283717, 283718, 283719, 283720, 283721, 283722, 283723, 283724, 283725, 283726, 283727, 283728, 283729) c6( ) c7(T, cF, c, eM, aW, ha, aT, PV, n, aNG, fd, fe, ci, K, cx, cm, V, af, aBs); #c1(VDAC2) c2(NP_001171712) c3(voltage-dependent anion-selective channel protein 2 isoform 1) c4(71381, 71382, 71383) c5(71384, 71385, 71386, 71387, 71388, 71389, 71390, 71391, 71392, 71393, 71394, 71395) c6( ) c7(qC, fM, aW, Y, ff, ha, aT, kf, h, aj, avF, zV, en, lC, bz, J, ip); #c1(VDAC2) c2(XP_011538430) c3(voltage-dependent anion-selective channel protein 2 isoform X1) c4(336191, 336192, 336193, 336194) c5(336195, 336196, 336197, 336198, 336199, 336200, 336201, 336202, 336203, 336204, 336205, 336206, 336207, 336208, 336209, 336210, 336211, 336212, 336213, 336214, 336215, 336216, 336217, 336218, 336219, 336220, 336221, 336222, 336223, 336224, 336225, 336226, 336227, 336228) c6( ) c7(qC, fM, aW, Y, ff, ha, aT, kf, h, aj, avF, zV, en, lC, bz, J, ip); #c1(VDR) c2(NP_000367) c3(vitamin D3 receptor isoform VDRA) c4(8159) c5(8160, 8161, 8162, 8163, 8164, 8165, 8166, 8167, 8168, 8169, 8170, 8171, 8172, 8173, 8174, 8175, 8176, 8177, 8178, 8179, 8180, 8181, 8182, 8183, 8184, 8185, 8186, 8187, 8188, 8189, 8190, 8191) c6( ) c7(bM, G, T, aC, fR, e, al, bB, cL, gf, qj, ci, bh, bbZ, aY, Tz, bOl, AW, dV, Ds, azm, rB, K, bbV, dF, bc, Zm, mH, aa, azh, aEV, zQ, aW, bsZ, hl, LZ, Qv, g, eE, eF, ba, xK, rq, Jr, nB, eM, iA, vA, U, cj, PV, vy, iS, azg, k, m, pw, lS, bi, qR, iu, FW, dD, bbW, aYZ, sw, aAP, aw, ht, NT, xJ, aj, fr, ai, amB, jz, zw, acm, aow, rl, cU, h, dL, aDp, aak, hi, cgB, Nr, Dg, mR, A, sp, OX, arp, ge, ks, bbX, aq, yq, po, CA, fe, zZ, cM, bJ, vU, KT, YE, sF, hk, at, uz, fL, it, B, rp, bL, vQ, fM, Ik, rG, bcb, eR, yk, oe, jx, cz, wo, hy, v, j, amC, C, iI, V, q, J, o, eA, MR, afQ, aT, aAR, jj, an, hK, ae, af, eu, bA, aRR, eC, qw, kl, s, iZ, Y, aCH, bg, r, iy, aDm, rs, Lc, en, bQ, t, aDQ, ck, ajl, arr, sS, OW, Dy, c, ac, BU, and, ig, iI, hr, hO, aeM, wR, dy, fD, bt, ars, gx, id, aFe, aZ, iW, axd, Ay, eq, jE, azk, mC, ad, kL, hX, dP, av, abk, hd, F, wD, yt, vJ, NS, oW, hD, Dj, JK, lQ, vB, ji, nJ, yF, sj, ul, BN, qt, PE, vj, ff, cr, eZ, aFr, hp, iV, HI, bz, dr, Nq, es, gk, yE, bE, bbY, aFb, aED, X, aef, bon, ei, asz, y, ne, sH, pC); #c1(VDR) c2(NP_001017536) c3(vitamin D3 receptor isoform VDRB1) c4(26595, 26596, 26597, 26598, 26599) c5(26600, 26601, 26602, 26603, 26604, 26605, 26606, 26607, 26608, 26609, 26610, 26611, 26612) c6( ) c7(bM, G, T, aC, fR, e, al, bB, c, gf, qj, ci, bh, bbZ, aY, Tz, bOl, AW, dV, Ds, azm, rB, K, bbV, dF, bc, Zm, mH, aa, azh, aEV, zQ, aW, bsZ, hl, LZ, Qv, g, eE, eF, ba, xK, rq, Jr, nB, eM, iA, vA, U, cj, PV, vy, iS, azg, k, m, pw, lS, bi, qR, iu, FW, dD, bbW, aYZ, sw, aAP, aw, ht, NT, xJ, aj, fr, ai, amB, jz, zw, acm, aow, rl, cU, h, dL, aDp, aak, hi, cgB, Nr, Dg, mR, A, sp, OX, arp, ge, ks, bbX, aq, yq, po, CQ, fe, zZ, cM, bJ, vU, KT, YE, sF, hk, at, uz, fL, it, B, rp, bL, vQ, fM, lk, rG, bcb, eR, yk, De, jx, cz, wo, hy, v, j, amC, C, iI, V, q, J, o, eA, MR, afQ, aT, aAR, jj, an, hk, ae, af, eu, bA, aRR, eC, qw, kl, s, iZ, Y, aCH, bg, r, iy, aDm, rs, Lc, en, bQ, t, aDQ, ck, ajl, arr, sS, OW, Dy, c, ac, BU, and, ig, iI, hr, hO, aeM, wR, dy, fD, bt, ars, gx, id, aFe, aZ, iW, axd, Ay, eq, jE, azk, mC, ad, kL, hX, dP, av, abk, hd, F, wD, yt, vJ, NS, oW, hD, Dj, JK, lQ, vB, ji, nJ, yF, sj, uQ, BN, qt, PE, vj, ff, cr, eZ, aFr, hp, iV, HI, bz, dr, Nq, es, gk, yE, bE, bbY, aFb, aED, X, aef, bon, ei, asz, y, ne, sH, pC); #c1(VEGFA) c2(NP_001020537) c3(vascular endothelial growth factor A isoform a) c4(28842, 28843, 28844) c5(28845, 28846, 28847, 28848, 28849, 28850, 28851, 28852, 28853, 28854, 28855) c6(28856, 28857) c7(T, bVx, ea, bO, fd, fo, aks, ci, jO, aY, Hr, bv, xy, aE, awe, fm, rw, Vv, hu, aCU, hH, aiJ, cp, aa, ly, yo, iE, wp, bvD, Ls, az, nY, fT, aZx, iA, nB, wE, cj, k, act, bvC, fy, uL, cE, bxC, RX, ht, sh, RC, cQ, aj, PJ, cd, Wy, adL, cty, Cf, bkP, bxi, ey, dd, pl, mR, anP, MD, aJ, Hz, fQ, fe, pm, iO, bkn, dB, bPc, bsO, Be, aoB, E, AN, DD, aA, cz, bX, lu, AO, q, bNX, oT, aWb, vz, MP, qK, cy, jj, sR, rv, dL, eu, le, iF, aGP, NE, nO, fO, bg, bsl, bcD, lo, Lc, cl, bkp, pb, ov, beH, iM, ac, ua, VA, aDR, PN, bt, ao, On, Mb, oq, eP, rQ, iW, uG, jN, jh, dG, uu, fu, xt, ad, hX, afb, aHF, iC, wD, yt, tT, cTl, oW, cTw, lQ, WZ, azH, ue, cH, PE, cW, ff, EH, EZ, iV, bz, DF, amS, aUN, aDJ, oQ, lC, lf, ne, jr, BA, iu, oO, bM, lD, bkx, if, e, bB, cL, lN, ip, aJO, x, blg, aeO, ja, cvG, xQ, eG, aeR, byj, Jl, fc, hl, Qq, kt, Qm, as, eE, eF, cm, bw, Qt, qJ, bb, xD, acS, go, vA, mx, vy, bru, tG, ym, in, bms, Vj, rt, dD, lT, bMe, n, oY, uE, Cd, EI, md, jz, Xe, gd, eV, bav, cY, A, RT, hT, PX, fJ, hQ, yq, YE, bP, cTF, aOL, hl, at, pg, ut, eo, sZ, B, cB, tq, dA, mr, eX, fF, v, Ge, Cb, V, eJ, Kx, cTH, RI, WY, BJ, rS, nQ, bau, ae, jf, zH, De, vR, vT, ev, blq, Qr, iZ, bSI, bu, t, ho, acp, vk, ck, vW, kP, lI, wl, gX, hO, yv, biL, hl, ob, Dd, dM, acl, asa, eq, adg, Fc, BY, hd, cTv, py, bNN, Db, Hk, LA, HD, bvM, nJ, sj, d, je, et, Z, arv, bF, gk, yE, bVu, ajw, lI, gZ, Hs, y, aC, UH, fR, al, axk, lR, Vp, cTK, gJ, aGm, xE, cTG, wM, Bc, pX, Qk, M, K, GO, wt, eL, adT, qS, LZ, aB, g, pL, uC, Vm, iP, AM, nF, U, PV, uo, QF, m, P, sQ, sw, Ol, aw, NT, qy, BH, pM, aCQ, cTC, eh, asS, JW, aky, ft, kH, cU, GT, Jn, ZN, Ha, dx, cTE, bsK, cTD, YF, cM, ar, KT, W, afc, bo, JM, bdA, gg, cTx, nz, Pr, it, fL, ayV, fM, wj, AL, KR, avm, fG, aGt, jx, zN, j, iI, uW, pd, eA, J, o, er, XX, Mg, anj, aHu, lg, bq, kl, PK, Y, r, en, wA, iK, Dm, Ci, bN, bmS, beJ, Dy, sn, c, hc, bxj, Da, np, dy, EY, fD, gT, Kg, bzT, gx, sY, kh, GI, id, Pt, aZ, oA, bS, ah, SY, hF, gb, buj, iJ, aAq, Jm, fU, dP, fX, sN, io, vB, hP, ji, aOh, Yi, uX, ael, BN, lR, anX, vj, eH, cr, si, VI, Fq, iR, Bu, bVw, qa, TA, Bv, Ew, nb, cgA, Fk, cP, ei, vt, G, Tb, auW, pf, eb, zh, aR, bU, cTJ, dQ, MO, eN, hm, DA, ayT, bvR, LC, aW, cnV, eM, iT, uJ, nw, mk, gn, R, Xu, cZ, tp, Wb, ko, fr, ct, bd, qN, ai, eW, Vs, Bl, boz, cc, hw, Yc, wG, kl, h, jy, dL, ux, hB, SD, vx, db, Um, wC, CM, iw, aq, kp, mo, gG, dw, op, lx, aoD, oU, bpC, cTz, gY, hk, cTA, Qu, bm, Vy, hR, aSW, uz, aAl, anx, bD, um, bL, eR, atB, dk, yk, rf, cR, tc, cJl, hy, Kh, C, cF, uT, nv, Vr, qp, afQ, HC, aT, dz, Fx, qk, af, qw, Uf, s, iI, aZB, oV, la, aaE, aDm, cTy, f, aDQ, wf, amF, wg, nM, akp, lV, AH, mf, VB, awY, hS, Ke, Zl, qb, aD, CI, Fb, tK, av, F, ot, aaU, Cj, ib, sO, NS, cTB, fb, ul, qt, eZ, Ej, VW, aF, aDd, ek, Hp, LB, es, bPZ, RV, pc, zr, bE, z, uR, Vw, aS, QD, be, XO, GF, tP, X, us); #c1(VEGFA) c2(NP_001020538) c3(vascular endothelial growth factor A isoform c) c4(28858, 28859, 28860) c5(28861, 28862, 28863, 28864, 28865, 28866, 28867, 28868, 28869, 28870) c6(28871, 28872) c7(T, bVx, ea, bO, fd, fo, aks, ci, jO, aY, Hr, bv, xy, aE, awe, fm, rw, Vv, hu, aCU, hH, aiJ, cp, aa, ly, yo, iE, wp, bvD, Ls, az, nY, fT, aZx, iA, nB, wE, cj, k, act, bvC, fy, ul, cE, bxC, RX, ht, sh, RC, cl, aj, PJ, cd, Wy, adL, cty, Cf, bkP, bxi, ey, dd, pl, mR, anP, MD, aJ, Hz, fQ, fe, pm, iO, bkn, dB, bPc, bsO, Be, aoB, E, AN, DD, aA, cz, bX, lu, AO, q, bNX, oT, aWb, vz, MP, qK, cy, jj, sR, rv, dL, eu, le, iF, aGP, NE, nO, fO, bg, bsl, bcD, lo, Lc, cl, bkp, pb, ov, beH, iM, ac, ua, VA, aDR, PN, bt, ao, On, Mb, oq, eP, rQ, iW, uG, jN, jh, dG, uu, fu, xt, ad, hX, afb, aHF, iC, wD, yt, tT, cTl, oW, cTw, lQ, WZ, azH, ue, cH, PE, cW, ff, EH, EZ, iV, bz, DF, amS, aUN, aDJ, oQ, lC, lf, ne, jr, BA, iu, oO, bM, lD, bkx, if, e, bB, cL, lN, ip, aJO, x, blg, aeO, ja, cvG, xQ, eG, aeR, byj, Jl, fc, hl, Qq, kt, Um, as, BE, eF, cm, bw, Qt, qJ, bb, xD, acS, go, vA, mx, vy, bru, tG, ym, in, bms, Vj, rt, dD, lT, bMe, n, oY, uE, Cd, El, md, jz, Xe, gd, eV, bav, cY, A, RT, hT, PX, fJ, hQ, yq, YE, bP, cTF, aOL, hl, at, pg, ut, eo, sZ, B, cB, tq, dA, mr, eX, fF, v, Ge, Cb, V, eJ, Kx, cTH, RI, WY, BJ, rS, nQ, bau, ae, jf, zH, De, vR, vT, ev, blq, Qr, iZ, bSI, bu, t, ho, acp, vk, ck, vW, kP, lI, wD, gX, hO, yv, biL, hl, ob, Dd, dM, acl, asa, eq, adg, Fc, BY, hd, cTv, py, bNN, Db, Hk, LA, hD, bvM, nJ, sj, d, je, et, Z, arv, bF, gk, yE, bVu, ajw, lI, gZ, Hs, y, aC, UH, fR, al, axk, lR, Vp, cTK, gJ, aGm, xE, cTG, wM, Bc, pX, Ok, M, K, GO, wt, eL, adT, qS, LZ, aB, g, pL, uC, Vm, iP, AM, nF, U, PV, uo, QF, m, P, sQ, sw, Ol, aw, NT, qy, BH, pM, aCQ, cTC, eh, asS, JW, aky, ft, KH, cU, GT, Jn, ZN, Ha, dx, cTE, bsk, cTD, YF, cM, ar, kT, W, afc, bo, JM, bdA, gg, cTx, nz, Pr, it, fL, ayV, fM, wj, AL, KR, avm, fG, aGt, jx, zN, j, iI, uW, pd, eA, J, o, er, XX, Mg, anj, aHu, lg, bq, kl, PK, Y, r, en, wA, iK, Dm, Ci, bN, bmS, beJ, Dy, sn, c, hc, bxj, Da, np, dy, EY, fD, gT, Kg, bzT, gx, sY, kh, GI, id, Pt, aZ, oA, bS, ah, SY, hF, gb, buj, iJ, aAq, Jm, fU, dP, fX, sN, io, vB, hP, ji, aOh, Yi, uX, ael, BN, lR, anX, vj, eH, cr, si, VI, Fq, iR, Bu, bVw, qa, TA, Bv, Ew, nb, cgA, Fk, cP, ei, vt, G, Tb, auW, pf, eb, zh, aR, bU, cTJ, dQ, MO, eN, hm, DA, ayT, bvR, LC, aW, cnV, eM, iT, uJ, nw, mk, gn, R, Xu, cZ, tp, Wb, ko, fr, ct, bd, qN, ai, eW, Vs, BI, boz, cc, hw, Yc, wG, kl, h, jy, dL, ux, hB, SD, vx, db, Um, wC, CM, iw, aq, kp, mo, gG, dw, op, lx, aoO, oU, bpC, cTz, gY, hk, cTA, Qu, bm, Vy, hR, aSW, uz, aAl, anx, bD, um, bL, eR, atB, dK, yk, rf, cR, tc, cJI, hy, Kh, C, cF, uT, nv, Vr, qp, afQ, HC, aT, dz, Fx, qk, af, qw, Uf, s, iU, aZB, oV, la, aaE, aDm, cTy, f, aDQ, wf, amF, wg, nM, akp, lV, AH, mf, VB, awY, hS, Ke, Zl, qb, aD, CI, Fb, tK, av, F, ot, aaU, Cj, ib, sO, NS, cTB, fb, ul, qt, eZ, Ej, VW, aF, aDd, ek, Hp, LB, es, bPZ, RV, pc, zr, bE, z, uR, Vw, aS, QD, be, XO, GF, tP, X, us); #c1(VEGFA) c2(NP_001020539) c3(vascular endothelial growth factor A isoform d) c4(28873, 28874, 28875, 28876) c5(28877, 28878, 28879, 28880, 28881, 28882, 28883, 28884, 28885, 28886) c6(28887, 28888) c7(T, bVx, ea, bO, fd, fo, aks, ci, jO, aY, Hr, bv, xy, aE, awe, fm, rw, Vv, hu, aCU, hH, aiJ, cp, aa, ly, yo, iE, wp, bvD, Ls, az, nY, fT, aZx, iA, nB, wE, cj, k, act, bvC, fy, ul, cE, bxC, RX, ht, sh, RC, cQ, aj, PJ, cd, Wy, adL, cty, Cf, bkP, bxi, ey, dd, pl, mR, anP, MD, aJ, Hz, fQ, fe, pm, iO, bkn, dB, bPc, bsO, Be, aoB, E, AN, DD, aA, cz, bX, lu, AO, q, bNX, oT, aWb, vz, MP, qK, cy, jj, sR, rv, dL, eu, le, if, aGP, NE, nO, fO, bg, bsl, bcD, lo, Lc, cl, bkp, pb, ov, beH, iM, ac, ua, VA, aDR, PN, bt, ao, On, Mb, oq, eP, rQ, iW, uG, jN, jh, dG, uu, fu, xt, ad, hX, afb, aHF, iC, wD, yt, tT, cTl, oW, cTw, lQ, WZ, azH, ue, cH, PE, cW, ff, EH, EZ, iV, bz, DF, amS, aUN, aDJ, oQ, lC, lf, ne, jr, BA, iu, oO, bM, lD, bkx, if, e, bB, cL, lN, ip, aJO, x, blg, aeO, ja, cvG, xQ, eG, aeR, byj, Jl, fc, hl, Qq, kt, lm, as, eE, eF, cm, bw, lt, qJ, bb, xD, acS, go, vA, mx, vy, bru, tG, ym, in, bms, Vj, rt, dD, lT, bMe, n, oY, uE, Cd, EI, md, jz, Xe, gd, eV, bav, cY, A, RT, hT, PX, fJ, hQ, yq, YE, bP, cTF, aOL, hl, at, pg, ut, eo, sZ, B, cB, tq, dA, mr, eX, fF, v, Ge, Cb, V, eJ, Kx, cTH, RI, WY, BJ, rS, nQ, bau, ae, jf, zH, De, vR, vT, ev, blq, Or, iZ, bSI, bu, t, ho, acp, vk, ck, vW, kP, lI, wD, gX, hO, yv, biL, hl, ob, Dd, dM, acl, asa, eq, adg, Fc, BY, hd, cTv, py, bNN, Db, Hk, LA, HD, bvM, nJ, sj, d, je, et, Z, arv, bF, gk, yE, bVu, ajw, lI, gZ, Hs, y, aC, UH, fR, al, axk, lR, Vp, cTK, gJ, aGm, xE, CTG, wM, Bc, pX, Qk, M, K, GO, wt, eL, adT, qS, LZ, aB, g, pL, uC, Vm, iP, AM, nF, U, PV, uo, QF, m, P, s, sw, Ol, aw, NT, qy, BH, pM, aCQ, cTC, eh, asS, JW, aky, ft, kH, cU, GT, Jn, ZN, Ha, dx, cTE, bsK, cTD, YF, cM, ar, kT, W, afc, bo, JM, bdA, gg, cTx, nz, Pr, it, fL, ayV, fM, wj, AL, KR, avm, fG, aGt, jx, zN, j, iI, uW, pd, eA, J, o, er, XX, Mg, anj, aHu, lg, bq, kl, PK, Y, r, en, wA, iK, Dm, Ci, bN, bmS, beJ, Dy, sn, c, hc, bxj, Da, np, dy, EY, fD, gT, Kg, bzT, gx, sY, kh, GI, id, Pt, aZ, oA, bS, ah, SY, hF, gb, buj, iJ, aAq, Jm, fU, dP, fX, sN, io, vB, hP, ji, aOh, Yi, uX, ael, BN, lR, anX, vj, eH, cr, si, VI, Fq, iR, Bu, bVw, qa, TA, Bv, Ew, nb, cgA, Fk, cP, ei, vt, G, Tb, auW, pf, eb, zh, aR, bll, cTJ, dQ, MO, eN, hm, DA, ayT, bvR, LC, aW, cnV, eM, iT, uJ, nw, mk, gn, R, Xu, cZ, tp, Wb, ko, fr, ct, bd, qN, ai, eW, Vs, BI, boz, cc, hw, Yc, wG, kl, h, jy, dL, ux, hB, SD, vx, db, Um, wC, CM, iw, aq, kp, mo, gG, dw, op, lx, aoO, oU, bpC, cTz, gY, hk, cTA, Qu, bm, Vy, hR, aSW, uz, aAl, anx, bD, um, bl, eR, atB, dk, yk, rf, cR, tc, cJl, hy, Kh, C, cF, uT, nv, Vr, qp, afQ, HC, aT, dz, Fx, qk, af, qw, Uf, s, iU, aZB, oV, la, aaE, aDm, cOy, f, aDQ, wf, amF, wg, nM, akp, lV, AH, mf, VB, awY, hS, Ke, Zl, qb, aD, CI, Fb, tK, av, F, ot, aaU, Cj, ib, sO, NS, cTB, fb, ul, qt, eZ, Ej, VW, aF, aDd, ek, Hp, LB, es, bPZ, RV, pc, zr, bE, z, uR, Vw, aS, QD, be, XO, GF, tP, X, us); #c1(VEGFA) c2(NP_001020540) c3(vascular endothelial growth factor A isoform e) c4(28889, 28890, 28891) c5(28892, 28893, 28894, 28895, 28896, 28897, 28898, 28899, 28900, 28901) c6(28902, 28903) c7(T, bVx, ea, bO, fd, fo, aks, ci, jO, aY, Hr, bv, xy, aE, awe, fm, rw, Vv, hu, aCU, hH, aiJ, cp, aa, ly, yo, iE, wp, bvD, Ls, az, nY, fT, aZx, iA, nB, wE, cj, k, act, bvC, fy, ul, cE, bxC, RX, ht, sh, RC, cQ, aj, PJ, cd, Wy, adL, cty, Cf, bkP, bxi, ey, dd, pl, mR, anP, MD, aJ, Hz, fQ, fe, pm, iO, bkn, dB, bPc, bsO, Be, aoB, E, AN, DD, aA, cz, bX, lu, AD, q, bNX, oT, aWb, vz, MP, qK, cy, jj, sR, rv, dL, eu, le, iF, aGP, NE, nO, fO, bg, bsl, bcD, lo, Lc, cl, bkp, pb, ov, beH, iM, ac, ua, VA, aDR, PN, bt, ao, On, Mb, oq, eP, rQ, iW, uG, jN, jh, dG, uu, fu, xt, ad, hX, afb, aHF, iC, wD, yt, tT, cTl, oW, cTw, lQ, WZ, azH, ue, CH, PE, cW, ff, EH, EZ, iV, bz, DF, amS, aUN, aDJ, oQ, lC, lf, ne, jr, BA, iu, oO, bM, lD, bkx, if, e, bB, cL, lN, ip, aJO, x, blg, aeO, ja, cvG, xQ, eG, aeR, byj, Jl, fc, hl, Qq, kt, lm, as, eE, eF, cm, bw, Qt, qJ, bb, xD, acS, go, vA, mx, vy, bru, tG, ym, in, bms, Vj, rt, dD, lT, bMe, n, oY, uE, Cd, EI, md, jz, Xe, gd, eV, bav, cY, A, RT, hT, PX, fJ, hQ, yq, YE, bP, cTF, aOL, hl, at, pg, ut, eo, sZ, B, cB, tq, dA, mr, ex, FF, v, Ge, Cb, V, eJ, Kx, cTH, RI, WY, BJ, rS, nQ, bau, ae, jf, zH, De, vR, vT, ev, blq, Qr, iZ, bSI, bu, t, ho, acp, vk, ck, vW, kP, lI, wl, gX, hO, yv, biL, hl, ob, Dd, dM, acl, asa, eq, adg, Fc, BY, hd, cTv, py, bNN, Db, Hk, LA, HD, bvM, nJ, sj, d, je, et, Z, arv, bF, gk, yE, bVu, ajw, lI, gZ, Hs, y, aC, UH, fR, al, axk, lR, Vp, cTK, gJ, aGm, xE, cTG, wM, Bc, pX, Ok, M, K, GO, wt, eL, adT, qS, LZ, aB, g, pL, uC, Vm, iP, AM, nF, U, PV, uo, QF, m, P, sQ, sw, OI, aw, NT, qy, BH, pM, aCQ, cTC, eh, asS, JW, aky, ft, kH, cU, GT, Jn, ZN, Ha, dx, cTE, bsk, cTD, YF, cM, ar, KT, W, afc, bo, JM, bdA, gg, clx, nz, Pr, it, fL, ayV, fM, wj, AL, KR, avm, fG, aGt, jx, zN, j, iI, uW, pd, eA, J, o, er, XX, Mg, anj, aHu, lg, bq, kl, PK, Y, r, en, wA, iK, Dm, Ci, bN, bmS, beJ, Dy, sn, c, hc, bxj, Da, np, dy, EY, fD, gT, Kg, bzT, gx, sY, kh, GI, id, Pt, aZ, oA, bS, ah, SY, hF, gb, buj, iJ, aAq, Jm, fU, dP, fX, sN, io, vB, hP, ji, aOh, Yi, uX, ael, BN, lR, anX, vj, eH, cr, si, VI, Fq, iR, Bu, bVw, qa, TA, Bv, Ew, nb, cgA, Fk, cP, ei, vt, G, Tb, auW, pf, eb, zh, aR, bll, cTJ, dQ, MO, eN, hm, DA, ayT, bvR, LC, aW, cnV, eM, iT, uJ, nw, mk, gn, R, Xu, cZ, tp, Wb, ko, fr, ct, bd, qN, ai, eW, Vs, BI, boz, cc, hw, Yc, wG, kl, h, jy, dL, ux, hB, SD, vx, db, Um, wC, CM, iw, aq, kp, mo, gG, dw, op, lx, aoD, oU, bpC, cTz, gY, hk, cTA, Qu, bm, Vy, hR, aSW, uz, aAl, anx, bD, um, bL, eR, atB, dk, yk, rf, cR, tc, cJl, hy, Kh, C, cF, uT, nv, Vr, qp, afQ, HC, aT, dz, Fx, qk, af, qw, Uf, s, iI, aZB, oV, la, aaE, aDm, cTy, f, aDQ, wf, amF, wg, nM, akp, lV, AH, mf, VB, awY, hS, Ke, Zl, qb, aD, CI, Fb, tK, av, F, ot, aaU, Cj, ib, sO, NS, cTB, fb, ul, qt, eZ, Ej, VW, aF, aDd, ek, Hp, LB, es, bPZ, RV, pc, zr, bE, z, uR, Vw, aS, QD, be, XO, GF, tP, X, us); #c1(VEGFA) c2(NP_001020541) c3(vascular endothelial growth factor A isoform f) c4(28904, 28905, 28906) c5(28907, 28908, 28909, 28910, 28911, 28912, 28913, 28914, 28915, 28916, 28917) c6(28918, 28919) c7(T, bVx, ea, bO, fd, fo, aks, ci, jO, aY, Hr, bv, xy, aE, awe, fm, rw, Vv, hu, aCU, hH, aiJ, cp, aa, ly, yo, iE, wp, bvD, Ls, az, nY, fT, aZx, iA, nB, wE, cj, k, act, bvC, fy, ul, cE, bxC, RX, ht, sh, RC, cQ, aj, PJ, cd, Wy, adL, cty, Cf, bkP, bxi, ey, dd, pl, mR, anP, MD, aJ, Hz, fQ, fe, pm, iO, bkn, dB, bPc, bsO, Be, aoB, E, AN, DD, aA, cz, bX, lu, AO, q, bNX, oT, aWb, vz, MP, qK, cy, jj, sR, rv, dL, eu, le, iF, aGP, NE, nO, fO, bg, bsl, bcD, lo, Lc, cl, bkp, pb, ov, beH, iM, ac, ua, VA, aDR, PN, bt, ao, On, Mb, oq, eP, rQ, iW, uG, jN, jh, dG, uu, fu, xt, ad, hX, afb, aHF, iC, wD, yt, tT, cTl, oW, cTw, IQ, WZ, azH, ue, cH, PE, cW, ff, EH, EZ, iV, bz, DF, amS, aUN, aDJ, oQ, lC, lf, ne, jr, BA, iu, oO, bM, lD, bkx, if, e, bB, cL, lN, ip, aJO, x, blg, ael, ja, cvG, xQ, eG, aeR, byj, Jl, fc, hl, Qq, kt, Qm, as, eE, eF, cm, bw, Qt, qJ, bb, xD, acS, go, vA, mx, vy, bru, tG, ym, in, bms, Vj, rt, dD, lT, bMe, n, oY, uE, Cd, EI, md, jz, Xe, gd, eV, bav, cY, A, RT, HT, PX, fJ, hQ, yq, YE, bP, cTF, aOL, hl, at, pg, ut, eo, sZ, B, cB, tq, dA, mr, ex, fF, v, Ge, Cb, V, eJ, Kx, cTH, RI, WY, BJ, rS, nQ, bau, ae, jf, zH, De, vR, vT, ev, blq, Or, iZ, bSI, bu, t, ho, acp, vk, ck, vW, kP, lI, wD, gX, hO, yv, biL, hl, ob, Dd, dM, acl, asa, eq, adg, Fc, BY, hd, cTv, py, bNN, Db, Hk, LA, hD, bvM, nJ, sj, d, je, et, Z, arv, bF, gk, yE, bVu, ajw, lI, gZ, Hs, y, aC, UH, fR, al, axk, IR, Vp, cTK, gJ, aGm, xE, cTG, wM, Bc, pX, Qk, M, K, GO, wt, eL, adT, qS, LZ, aB, g, pL, uC, Vm, iP, AM, nF, U, PV, uo, QF, m, P, sQ, sw, Ol, aw, NT, qy, BH, pM, aCQ, cTC, eh, asS, JW, aky, ft, kH, cU, GT, Jn, ZN, Ha, dx, cTE, bsK, cTD, YF, cM, ar, KT, W, afc, bo, JM, bdA, gg, cTx, nz, Pr, it, fl, ayV, fM, wj, AL, KR, avm, fG, aGt, jx, zN, j, iI, uW, pd, eA, J, o, er, XX, Mg, anj, aHu, lg, bq, kl, PK, Y, r, en, wA, iK, Dm, Ci, bN, bmS, beJ, Dy, sn, c, hc, bxj, Da, np, dy, EY, fD, gT, Kg, bzT, gx, sY, kh, GI, id, Pt, aZ, oA, bS, ah, SY, hF, gb, buj, iJ, aAq, Jm, fU, dP, fX, sN, io, vB, hP, ji, aOh, Yi, uX, aed, BN, lR, anX, vj, eH, cr, si, VI, Fq, iR, Bu, bVw, qa, TA, Bv, Ew, nb, cgA, Fk, cP, ei, vt, G, Tb, auW, pf, eb, zh, aR, bll, cTJ, dQ, MO, eN, hm, DA, ayT, bvR, LC, aW, cnV, eM, iT, uJ, nw, mk, gn, R, Xu, cZ, tp, Wb, ko, fr, ct, bd, qN, ai, eW, Vs, BI, boz, cc, hw, Yc, wG, kl, h, jy, dL, ux, hB, SD, vx, db, Um, wC, CM, iw, aq, kp, mo, gG, dw, op, lx, aoO, ou, bpC, cTz, gY, hk, cTA, Qu, bm, Vy, hR, aSW, uz, aAl, anx, bD, um, bL, eR, atB, dK, yk, rf, cR, tc, cJl, hy, Kh, C, cF, uT, nv, Vr, qp, afQ, HC, aT, dz, Fx, qk, af, qw, Uf, s, iU, aZB, oV, la, aaE, aDm, cTy, f, aDQ, wf, amF, wg, nM, akp, lV, AH, mf, VB, awY, hS, Ke, Zl, qb, aD, CI, Fb, tK, av, F, ot, aaU, Cj, ib, sO, NS, cTB, fb, ud, qt, eZ, Ej, VW, aF, aDd, ek, Hp, LB, es, bPZ, RV, pc, zr, bE, z, uR, Vw, aS, QD, be, XO, GF, tP, X, us); #c1(VEGFA) c2(NP_001028928) c3(vascular endothelial growth factor A isoform g) c4(30702, 30703, 30704) c5(30705, 30706, 30707, 30708, 30709, 30710, 30711, 30712, 30713, 30714) c6(30715, 30716) c7(T, bVx, ea, b0, fd, fo, aks, ci, jO, aY, Hr, bv, xy, aE, awe, fm, rw, Vv, hu, aCU, hH, aiJ, cp, aa, ly, yo, iE, wp, bvD, Ls, az, nY, fT, aZx, iA, nB, wE, cj, k, act, bvC, fy, uL, cE, bxC, RX, ht, sh, RC, cQ, aj, PJ, cd, Wy, adL, cty, Cf, bkP, bxi, ey, dd, pl, mR, anP, MD, aJ, Hz, fQ, fe, pm, iO, bkn, dB, bPc, bsO, Be, aoB, E, AN, DD, aA, cz, bX, lu, AO, q, bNX, oT, aWb, vz, MP, qK, cy, jj, sR, rv, dL, eu, le, iF, aGP, NE, nO, fO, bg, bsl, bcD, lo, Lc, cl, bkp, pb, ov, beH, iM, ac, ua, VA, aDR, PN, bt, ao, On, Mb, oq, eP, rQ, iW, uG, jN, jh, dG, uu, fu, xt, ad, hX, afb, aHF, iC, wD, yt, tT, cTl, oW, cTw, lQ, WZ, azH, ue, cH, PE, cW, ff, EH, EZ, iV, bz, DF, amS, aUN, aDJ, oQ, lC, lf, ne, jr, BA, iu, oO, bM, lD, bkx, if, e, bB, cL, lN, ip, aJO, x, blg, aeO, ja, cvG, xQ, eG, aeR, byj, Jl, fc, hl, Qq, kt, Um, as, eE, eF, cm, bw, Qt, qJ, bb, xD, acS, go, vA, mx, vy, bru, tG, ym, in, bms, Vj, rt, dD, lT, bMe, n, oY, uE, Cd, EI, md, jz, Xe, gd, eV, bav, cY, A, RT, hT, PX, fJ, hQ, yq, YE, bP, cTF, aOL, hL, at, pg, ut, eo, sZ, B, cB, tq, dA, mr, eX, fF, v, Ge, Cb, V, eJ, Kx, cTH, RI, WY, BJ, rS, nd, bau, ae, jf, zH, De, vR, vT, ev, blq, Qr, iZ, bSI, bu, t, ho, acp, vk, ck, vW, kP, lI, wD, gX, hO, yv, biL, hl, ob, Dd, dM, acl, asa, eq, adg, Fc, BY, hd, cTv, py, bNN, Db, Hk, LA, hD, bvM, nJ, sj, d, je, et, Z, arv, bF, gk, yE, bVu, ajw, lI, gZ, Hs, y, aC, UH, fR, al, axk, lR, Vp, cTK, gJ, aGm, xE, cTG, wM, Bc, pX, Qk, M, K, GO, wt, eL, adT, qS, LZ, aB, g, pL, uC, Vm, iP, AM, nF, U, PV, uo, QF, m, P, sQ, sw, OI, aw, NT, qy, BH, pM, aCD, cTC, eh, asS, JW, aky, ft, kH, cU, GT, Jn, ZN, Ha, dx, cTE, bsK, cTD, YF, cM, ar, kT, W, afc, bo, JM, bdA, gg, cTx, nz, Pr, it, fL, ayV, fM, wj, AL, KR, avm, fG, aGt, jx, zN, j, iI, uW, pd, eA, J, o, er, XX, Mg, anj, aHu, lg, bq, kl, PK, Y, r, en, wA, iK, Dm, Ci, bN, bmS, beJ, Dy, sn, c, hc, bxj, Da, np, dy, EY, fD, gT, Kg, bzT, gx, sY, kh, GI, id, Pt, aZ, oA, bS, ah, SY, hF, gb, buj, iJ, aAq, Jm, fU, dP, fX, sN, io, vB, hP, ji, aOh, Yi, uX, ael, BN, lR, anX, vj, eH, cr, si, VI, Fq, iR, Bu, bVw, qa, TA, Bv, Ew, nb, cgA, Fk, cP, ei, vt, G, Tb, auW, pf, eb, zh, aR, bll, cTJ, dQ, MO, eN, hm, DA, ayT, bvR, LC, aW, cnV, eM, iT, uJ, nw, mk, gn, R, Xu, cZ, tp, Wb, ko, fr, ct, bd, qN, ai, eW, Vs, BI, boz, cc, hw, Yc, wG, kl, h, jy, dL, ux, hB, SD, vx, db, Um, WC, CM, iw, aq, kp, mo, gG, dw, op, lx, aoD, oU, bpC, cTz, gY, hk, cTA, Qu, bm, Vy, hR, aSW, uz, aAl, anx, bD, um, bL, eR, atB, dk, yk, rf, cR, tc, cJl, hy, Kh, C, cF, uT, nv, Vr, qp, afQ, HC, aT, dz, Fx, qk, af, qw, Uf, s, iU, aZB, oV, la, aaE, aDm, cTy, f, aDQ, wf, amF, wg, nM, akp, lV, AH, mf, VB, awY, hS, Ke, Zl, qb, aD, CI, Fb, tK, av, F, ot, aaU, Cj, ib, sO, NS, cTB, fb, uQ, qt, eZ, Ej, VW, aF, aDd, ek, Hp, LB, es, bPZ, RV, pc, zr, bE, z, uR, Vw, aS, QD, be, XO, GF, tP, X, us); #c1(VEGFA) c2(NP_001165093) c3(vascular endothelial growth factor A isoform h) c4(67290, 67291, 67292) c5(67293, 67294, 67295, 67296, 67297, 67298, 67299, 67300, 67301, 67302) c6(67303, 67304) c7(T, bVx, ea, bO, fd, fo, aks, ci, jO, aY, Hr, bv, xy, aE, awe, fm, rw, Vv, hu, aCU, hH, aiJ, cp, aa, ly, yo, iE, wp, bvD, Ls, az, nY, fT, aZx, iA, nB, wE, cj, k, act, bvC, fy, ul, cE, bxC, RX, ht, sh, RC, cQ, aj, PJ, cd, Wy, adL, cty, Cf, bkP, bxi, ey, dd, pl, mR, anP, MD, aJ, Hz, fQ, fe, pm, iO, bkn, dB, bPc, bsO, Be, aoB, E, AN, DD, aA, cz, bX, lu, AO, q, bNX, oT, aWb, vz, MP, qK, cy, jj, sR, rv, dL, eu, le, if, aGP, NE, nO, fO, bg, bsl, bcD, lo, Lc, cl, bkp, pb, ov, beH, iM, ac, ua, VA, aDR, PN, bt, ao, On, Mb, oq, eP, rQ, iW, uG, jN, jh, dG, uu, fu, xt, ad, hX, afb, aHF, iC, wD, yt, tT, cTl, oW, cTw, lQ, WZ, azH, ue, CH, PE, cW, ff, EH, EZ, iV, bz, DF, amS, aUN, aDJ, oQ, lC, lf, ne, jr, BA, iu, oO, bM, lD, bkx, if, e, bB, cL, lN, ip, aJO, x, blg, aeO, ja, cvG, xQ, eG, aeR, byj, Jl, fc, hl, Qq, kt, Qm, as, eE, eF, cm, bw, Qt, qJ, bb, xD, acS, go, vA, mx, vy, bru, tG, ym, in, bms, Vj, rt, dD, lT, bMe, n, oY, uE, Cd, EI, md, jz, Xe, gd, eV, bav, cY, A, RT, hT, PX, fJ, hQ, yq, YE, bP, cTF, aOL, hl, at, pg, ut, eo, sZ, B, cB, tq, dA, mr, eX, fF, v, Ge, Cb, V, eJ, Kx, cTH, RI, WY, BJ, rS, nd, bau, ae, jf, zH, De, vR, vT, ev, blq, Or, iZ, bSI, bu, t, ho, acp, vk, ck, vW, kP, lI, wl, gX, hO, yv, biL, hl, ob, Dd, dM, acl, asa, eq, adg, Fc, BY, hd, cTv, py, bNN, Db, Hk, LA, HD, bvM, nJ, sj, d, je, et, Z, arv, bF, gk, yE, bVu, ajw, lI, gZ, Hs, y, aC, UH, fR, al, axk, IR, Vp, cTK, gJ, aGm, xE, cTG, wM, Bc, pX, Qk, M, K, GO, wt, eL, adT, qS, LZ, aB, g, pL, uC, Vm, iP, AM, nF, U, PV, uo, QF, m, P, sQ, sw, Ol, aw, NT, qy, BH, pM, aCQ, cTC, eh, asS, JW, aky, ft, kH, cU, GT, Jn, ZN, Ha, dx, cTE, bsk, cTD, YF, CM, ar, kT, W, afc, bo, JM, bdA, gg, cTx, nz, Pr, it, fl, ayV, fM, wj, AL, KR, avm, fG, aGt, jx, zN, j, iI, uW, pd, eA, J, o, er, XX, Mg, anj, aHu, lg, bq, kl, PK, Y, r, en, wA, iK, Dm, Ci, bN, bmS, bel, Dy, sn, c, hc, bxj, Da, np, dy, EY, fD, gT, Kg, bzT, gx, sY, kh, GI, id, Pt, aZ, oA, bS, ah, SY, hF, gb, buj, iJ, aAq, Jm, fU, dP, fX, sN, io, vB, hP, ji, aOh, Yi, uX, aed, BN, lR, anX, vj, eH, cr, si, VI, Fq, iR, Bu, bVw, qa, TA, Bv, Ew, nb, cgA, Fk, cP, ei, vt, G, Tb, auW, pf, eb, zh, aR, bll, cTJ, dQ, MO, eN, hm, DA, ayT, bvR, LC, aW, cnV, eM, iT, uJ, nw, mk, gn, R, Xu, cZ, tp, Wb, ko, fr, ct, bd, qN, ai, eW, Vs, BI, boz, cc, hw, Yc, wG, kl, h, jy, dL, ux, hB, SD, vx, db, Um, wC, CM, iw, aq, kp, mo, gG, dw, op, lx, aoO, oU, bpC, cTz, gY, hk, cTA, Qu, bm, Vy, hR, aSW, uz, aAl, anx, bD, um, bl, eR, atB, dk, yk, rf, cR, tc, cJl, hy, Kh, C, cF, uT, nv, Vr, qp, afQ, HC, aT, dz, Fx, qk, af, qw, Uf, s, iI, aZB, oV, la, aaE, aDm, cTy, f, aDQ, wf, amF, wg, nM, akp, lV, AH, mf, VB, awY, hS, Ke, Zl, qb, aD, CI, Fb, tK, av, F, ot, aaU, Cj, ib, sO, NS, cTB, fb, ud, qt, eZ, Ej, VW, aF, aDd, eK, Hp, LB, es, bPZ, RV, pc, zr, bE, z, uR, Vw, aS, QD, be, XO, GF, tP, X, us); #c1(VEGFA) c2(NP_001165094) c3(vascular endothelial growth factor A isoform i precursor) c4( ) c5(67305, 67306, 67307, 67308, 67309, 67310, 67311, 67312) c6(67313, 67314) c7(T, bVx, ea, bO, fd, fo, aks, ci, jO, aY, Hr, bv, xy, aE, awe, fm, rw, Vv, hu, aCU, hH, aiJ, cp, aa, ly, yo, iE, wp, bvD, Ls, az, nY, fT, aZx, iA, nB, wE, cj, k, act, bvC, fy, ul, cE, bxC, RX, ht, sh, RC, cQ, aj, PJ, cd, Wy, adL, cty, Cf, bkP, bxi, ey, dd, pl, mR, anP, MD, aJ, Hz, fQ, fe, pm, iO, bkn, dB, bPc, bsO, Be, aoB, E, AN, DD, aA, cz, bX, lu, AD, q, bNX, oT, aWb, vz, MP, qK, cy, jj, sR, rv, dL, eu, le, iF, aGP, NE, nO, fO, bg, bsl, bcD, lo, Lc, cl, bkp, pb, ov, beH, iM, ac, ua, VA, aDR, PN, bt, ao, On, Mb, oq, eP, rQ, iW, uG, jN, jh, dG, uu, fu, xt, ad, hX, afb, aHF, iC, wD, yt, tT, cTl, oW, cTw, lQ, WZ, azH, ue, cH, PE, cW, ff, EH, EZ, iV, bz, DF, amS, aUN, aDJ, od, lC, lf, ne, jr, BA, iu, oO, bM, lD, bkx, if, e, bB, cL, lN, ip, aJO, x, blg, aeO, ja, cvG, xQ, eG, aeR, byj, Jl, fc, hl, Qq, kt, lm, as, eE, eF, cm, bw, Qt, qJ, bb, xD, acS, go, vA, mx, vy, bru, tG, ym, in, bms, Vj, rt, dD, lT, bMe, n, oY, uE, Cd, EI, md, jz, Xe, gd, eV, bav, CY, A, RT, hT, PX, fJ, hQ, yq, YE, bP, cTF, aOL, hL, at, pg, ut, eo, sZ, B, cB, tq, dA, mr, eX, fF, v, Ge, Cb, V, eJ, Kx, cTH, RI, WY, BJ, rS, nQ, bau, ae, jf, zH, De, vR, vT, ev, blq, Qr, iZ, bSI, bu, t, ho, acp, vk, ck, vW, kP, lI, wD, gX, hO, yv, biL, hl, ob, Dd, dM, acl, asa, eq, adg, Fc, BY, hd, cTv, py, bNN, Db, Hk, LA, HD, bvM, nJ, sj, d, je, et, Z, arv, bF, gk, yE, bVu, ajw, lI, gZ, Hs, y, aC, UH, fR, al, axk, lR, Vp, cTK, gJ, aGm, xE, cTG, wM, Bc, pX, Ok, M, K, GO, wt, eL, adT, qS, LZ, aB, g, pL, uC, Vm, iP, AM, nF, U, PV, uo, QF, m, P, sQ, sw, Ol, aw, NT, qy, BH, pM, aCQ, cTC, eh, asS, JW, aky, ft, kH, cU, GT, Jn, ZN, Ha, dx, cTE, bsk, cTD, YF, cM, ar, kT, W, afc, bo, JM, bdA, gg, cTx, nz, Pr, it, fL, ayV, fM, wj, AL, KR, avm, fG, aGt, jx, zN, j, iI, uW, pd, eA, J, o, er, XX, Mg, anj, aHu, lg, bq, kl, PK, Y, r, en, wA, iK, Dm, Ci, bN, bmS, beJ, Dy, sn, c, hc, bxj, Da, np, dy, EY, fD, gT, Kg, bzT, gx, sY, kh, GI, id, Pt, aZ, oA, bS, ah, SY, hF, gb, buj, iJ, aAq, Jm, fU, dP, fX, sN, io, vB, hP, ji, aOh, Yi, uX, aeQ, BN, lR, anX, vj, eH, cr, si, VI, Fq, iR, Bu, bVw, qa, TA, Bv, Ew, nb, cgA, Fk, cP, ei, vt, G, Tb, auW, pf, eb, zh, aR, bll, cTJ, dQ, MO, eN, hm, DA, ayT, bvR, LC, aW, cnV, BM, iT, uJ, nw, mk, gn, R, Xu, cZ, tp, Wb, ko, fr, ct, bd, qN, ai, eW, Vs, BI, boz, cc, hw, Yc, wG, kl, h, jy, dL, ux, hB, SD, vx, db, Um, WC, CM, iw, aq, kp, mo, gG, dw, op, lx, aoO, oU, bpC, cTz, gY, hk, cTA, Qu, bm, Vy, hR, aSW, uz, aAl, anx, bD, um, bL, eR, atB, dk, yk, rf, cR, tc, cJI, hy, Kh, C, cF, uT, nv, Vr, qp, afQ, HC, aT, dz, Fx, qk, af, qw, Uf, s, iU, aZB, oV, la, aaE, aDm, cTy, f, aDQ, wf, amF, wg, nM, akp, lV, AH, mf, VB, awY, hS, Ke, Zl, qb, aD, CI, Fb, tK, av, F, ot, aaU, Cj, ib, sO, NS, cTB, fb, uQ, qt, eZ, Ej, VW, aF, aDd, ek, Hp, LB, es, bPZ, RV, pc, zr, bE, z, uR, Vw, aS, QD, be, XO, GF, tP, X, us); #c1(VEGFA) c2(NP_001165095) c3(vascular endothelial growth factor A isoform j precursor) c4( ) c5(67315, 67316, 67317, 67318, 67319, 67320, 67321, 67322) c6(67323, 67324) c7(T, bVx, ea, b, fd, fo, aks, ci, jO, aY, Hr, bv, xy, aE, awe, fm, rw, Vv, hu, aCU, hH, aiJ, cp, aa, ly, yo, iE, wp, bvD, Ls, az, nY, fT, aZx, iA, nB, wE, cj, k, act, bvC, fy, ul, cE, bxC, RX, ht, sh, RC, cQ, aj, PJ, cd, Wy, adL, cty, Cf, bkP, bxi, ey, dd, pl, mR, anP, MD, aJ, Hz, fQ, fe, pm, iO, bkn, dB, bPc, bsO, Be, aoB, E, AN, DD, aA, cz, bX, lu, AO, q, bNX, oT, aWb, vz, MP, qK, cy, jj, sR, rv, dL, eu, le, iF, aGP, NE, nO, fO, bg, bsl, bcD, lo, Lc, cl, bkp, pb, ov, beH, iM, ac, ua, VA, aDR, PN, bt, ao, On, Mb, oq, eP, rQ, iW, uG, jN, jh, dG, uu, fu, xt, ad, hX, afb, aHF, iC, wD, yt, tT, cTl, oW, cTw, lQ, WZ, azH, ue, cH, PE, cW, ff, EH, EZ, iV, bz, DF, amS, aUN, aDJ, ol, IC, lf, ne, jr, BA, iu, oO, bM, lD, bkx, if, e, bB, cL, lN, ip, aJO, x, blg, aeO, ja, cvG, xQ, eG, aeR, byj, Jl, fc, hl, Qq, kt, Qm, as, eE, eF, cm, bw, Qt, qJ, bb, xD, acS, go, vA, mx, vy, bru, tG, ym, in, bms, Vj, rt, dD, lT, bMe, n, oY, uE, Cd, EI, md, jz, Xe, gd, eV, bav, cY, A, RT, hT, PX, fJ, hQ, yq, YE, bP, cTF, aOL, hL, at, pg, ut, eo, sZ, B, cB, tq, dA, mr, eX, FF, v, Ge, Cb, V, eJ, Kx, cTH, RI, WY, BJ, rS, nQ, bau, ae, jf, zH, De, vR, vT, ev, blq, Ar, iZ, bSI, bu, t, ho, acp, vk, ck, vW, kP, lI, wD, gX, hO, yv, biL, hl, ob, Dd, dM, acl, asa, eq, adg, Fc, BY, hd, cTv, py, bNN, Db, Hk, LA, hD, bvM, nJ, sj, d, je, et, Z, arv, bF, gk, yE, bVu, ajw, lI, gZ, Hs, y, aC, UH, fR, al, axk, lR, Vp, cTK, gJ, aGm, xE, cTG, wM, Bc, pX, Qk, M, K, GO, wt, eL, adT, qS, LZ, aB, g, pL, uC, Vm, iP, AM, nF, U, PV, uo, QF, m, P, sQ, sw, Ol, aw, NT, qy, BH, pM, aCQ, cTC, eh, asS, JW, aky, ft, kH, cU, GT, Jn, ZN, Ha, dx, cTE, bsk, cTD, YF, cM, ar, kT, W, afc, bo, JM, bdA, gg, cTx, nz, Pr, it, fL, ayV, fM, wj, AL, KR, avm, fG, aGt, jx, zN, j, iI, uW, pd, eA, J, o, er, XX, Mg, anj, aHu, lg, bq, kl, PK, Y, r, en, wA, iK, Dm, Ci, bN, bmS, beJ, Dy, sn, c, hc, bxj, Da, np, dy, EY, fD, gT, Kg, bzT, gx, sY, kh, GI, id, Pt, aZ, DA, bS, ah, SY, hF, gb, buj, iJ, aAq, Jm, fU, dP, fX, sN, io, vB, hP, ji, aOh, Yi, uX, aeQ, BN, lR, anX, vj, eH, cr, si, VI, Fq, iR, Bu, bVw, qa, TA, Bv, Ew, nb, cgA, Fk, cP, ei, vt, G, Tb, auW, pf, eb, zh, aR, bll, cTJ, dQ, MO, eN, hm, DA, ayT, bvR, LC, aW, cnV, eM, iT, uJ, nw, mk, gn, R, Xu, cZ, tp, Wb, ko, fr, ct, bd, qN, ai, eW, Vs, BI, boz, cc, hw, Yc, wG, kl, h, jy, dL, ux, hB, SD, vx, db, Um, wC, CM, iw, aq, kp, mo, gG, dw, op, lx, aoD, oU, bpC, cTz, gY, hk, cTA, Qu, bm, Vy, hR, aSW, uz, aAl, anx, bD, um, bL, eR, atB, dK, yk, rf, cR, tc, cJl, hy, Kh, C, cF, uT, nv, Vr, qp, afQ, HC, aT, dz, Fx, qk, af, qw, Uf, s, iI, aZB, oV, la, aaE, aDm, cTy, f, aDQ, wf, amF, wg, nM, akp, lV, AH, mf, VB, awY, hS, Ke, Zl, qb, aD, CI, Fb, tK, av, F, ot, aaU, Cj, ib, sO, NS, cTB, fb, uQ, qt, eZ, Ej, VW, aF, aDd, ek, Hp, LB, es, bPZ, RV, pc, zr, bE, z, uR, Vw, aS, QD, be, XO, GF, tP, X, us); #c1(VEGFA) c2(NP_001165096) c3(vascular endothelial growth factor A isoform k precursor) c4( ) c5(67325, 67326, 67327, 67328, 67329, 67330, 67331, 67332, 67333) c6(67334, 67335) c7(T, bVx, ea, bO, fd, fo, aks, ci, jO, aY, Hr, bv, xy, aE, awe, fm, rw, Vv, hu, aCU, hH, aiJ, cp, aa, ly, yo, iE, wp, bvD, Ls, az, nY, fT, aZx, iA, nB, wE, cj, k, act, bvC, fy, uL, cE, bxC, RX, ht, sh, RC, cQ, aj, PJ, cd, Wy, adL, cty, Cf, bkP, bxi, ey, dd, pl, mR, anP, MD, aJ, Hz, fQ, fe, pm, iO, bkn, dB, bPc, bsO, Be, aoB, E, AN, DD, aA, cz, bX, lu, A, q, bNX, oT, aWb, vz, MP, qK, cy, jj, sR, rv, dL, eu, Ie, iF, aGP, NE, nO, fO, bg, bsl, bcD, lo, Lc, cl, bkp, pb, ov, beH, iM, ac, ua, VA, aDR, PN, bt, ao, On, Mb, oq, eP, rQ, iW, uG, jN, jh, dG, uu, fu, xt, ad, hX, afb, aHF, iC, wD, yt, tT, cTl, oW, cTw, lQ, WZ, azH, ue, CH, PE, cW, ff, EH, EZ, iV, bz, DF, amS, aUN, aDJ, od, lC, lf, ne, jr, BA, iu, oO, bM, ID, bkx, if, e, bB, cL, lN, ip, aJO, x, blg, aeO, ja, cvG, xQ, eG, aeR, byj, Jl, fc, hl, Qq, kt, Qm, as, eE, eF, cm, bw, Qt, qJ, bb, xD, acS, go, vA, mx, vy, bru, tG, ym, in, bms, Vj, rt, dD, lT, bMe, n, oY, uE, Cd, EI, md, jz, Xe, gd, eV, bav, cY, A, RT, hT, PX, fJ, hQ, yq, YE, bP, cTF, aOL, hL, at, pg, ut, eo, sZ, B, cB, tq, dA, mr, eX, fF, v, Ge, Cb, V, eJ, Kx, cTH, RI, WY, BJ, rS, nQ, bau, ae, jf, zH, De, vR, vT, ev, blq, Qr, iZ, bSI, bu, t, ho, acp, vk, ck, vW, kP, lI, wD, gX, hO, yv, biL, hl, ob, Dd, dM, acl, asa, eq, adg, Fc, BY, hd, cTv, py, bNN, Db, Hk, LA, HD, bvM, nJ, sj, d, je, et, Z, arv, bF, gk, yE, bVu, ajw, lI, gZ, Hs, y, aC, UH, fR, al, axk, lR, Vp, cTK, gJ, aGm, xE, cTG, wM, Bc, pX, Qk, M, K, GO, wt, eL, adT, qS, LZ, aB, g, pL, uC, Vm, iP, AM, nF, U, PV, uo, QF, m, P, sQ, sw, OI, aw, NT, qy, BH, pM, aCQ, cTC, eh, asS, JW, aky, ft, kH, cU, GT, Jn, ZN, Ha, dx, cTE, bsk, cTD, YF, cM, ar, kT, W, afc, bo, JM, bdA, gg, clx, nz, Pr, it, fL, ayV, fM, wj, AL, KR, avm, fG, aGt, jx, zN, j, iI, uW, pd, eA, J, o, er, XX, Mg, anj, aHu, lg, bq, kl, PK, Y, r, en, wA, iK, Dm, Ci, bN, bmS, beJ, Dy, sn, c, hc, bxj, Da, np, dy, EY, fD, gT, Kg, bzT, gx, sY, kh, GI, id, Pt, aZ, oA, bS, ah, SY, hF, gb, buj, iJ, aAq, Jm, fU, dP, fX, sN, io, vB, hP, ji, aOh, Yi, uX, aeQ, BN, lR, anX, vj, eH, cr, si, VI, Fq, iR, Bu, bVw, qa, TA, Bv, Ew, nb, cgA, Fk, cP, ei, vt, G, Tb, auW, pf, eb, zh, aR, bll, cTJ, dQ, MO, eN, hm, DA, ayT, bvR, LC, aW, cnV, eM, iT, uJ, nw, mk, gn, R, Xu, cZ, tp, Wb, ko, fr, ct, bd, qN, ai, eW, Vs, Bl, boz, cc, hw, Yc, wG, kl, h, jy, dL, ux, hB, SD, vx, db, Um, wC, CM, iw, aq, kp, mo, gG, dw, op, lx, aoD, oU, bpC, cTz, gY, hk, cTA, Qu, bm, Vy, hR, aSW, uz, aAl, anx, bD, um, bL, eR, atB, dk, yk, rf, cR, tc, cJl, hy, Kh, C, cF, uT, nv, Vr, qp, afQ, HC, aT, dz, Fx, qk, af, qw, Uf, s, iI, aZB, oV, la, aaE, aDm, cTy, f, aDQ, wf, amF, wg, nM, akp, lV, AH, mf, VB, awY, hS, Ke, Zl, qb, aD, CI, Fb, tK, av, F, ot, aaU, Cj, ib, sO, NS, cTB, fb, ul, qt, eZ, Ej, VW, aF, aDd, ek, Hp, LB, es, bPZ, RV, pc, zr, bE, z, uR, Vw, aS, QD, be, XO, GF, tP, X, us); #c1(VEGFA) c2(NP_001165097) c3(vascular endothelial growth factor A isoform 1 precursor) c4( ) c5(67336, 67337, 67338, 67339, 67340, 67341, 67342, 67343, 67344, 67345, 67346, 67347, 67348, 67349, 67350, 67351, 67352, 67353, 67354, 67355, 67356, 67357, 67358, 67359, 67360, 67361, 67362, 67363, 67364, 67365, 67366, 67367, 67368, 67369, 67370, 67371, 67372, 67373, 67374, 67375, 67376, 67377, 67378, 67379, 67380, 67381, 67382, 67383, 67384, 67385, 67386, 67387, 67388, 67389, 67390, 67391, 67392, 67393, 67394, 67395, 67396, 67397, 67398, 67399, 67400, 67401, 67402, 67403, 67404, 67405, 67406, 67407, 67408, 67409, 67410, 67411, 67412, 67413, 67414, 67415, 67416, 67417, 67418, 67419, 67420, 67421, 67422, 67423, 67424, 67425, 67426, 67427, 67428, 67429, 67430, 67431, 67432) c6(67433, 67434, 67435) c7(T, bVx, ea, bO, fd, fo, aks, ci, jO, aY, Hr, bv, xy, aE, awe, fm, rw, Vv, hu, aCU, hH, aiJ, cp, aa, ly, yo, iE, wp, bvD, Ls, az, nY, fT, aZx, iA, nB, wE, cj, k, act, bvC, fy, ul, cE, bxC, RX, ht, sh, RC, cQ, aj, PJ, cd, Wy, adL, cty, Cf, bkP, bxi, ey, dd, pl, mR, anP, MD, aJ, Hz, fQ, fe, pm, iO, bkn, dB, bPc, bsO, Be, aoB, E, AN, DD, aA, cz, bX, lu, AO, q, bNX, oT, aWb, vz, MP, qK, cy, jj, sR, rv, dL, eu, le, if, aGP, NE, nO, fO, bg, bsl, bcD, lo, Lc, cl, bkp, pb, ov, beH, iM, ac, ua, VA, aDR, PN, bt, ao, On, Mb, oq, eP, rQ, iW, uG, jN, jh, dG, uu, fu, xt, ad, hX, afb, aHF, iC, wD, yt, tT, cTl, oW, cTw, lQ, WZ, azH, ue, cH, PE, cW, ff, EH, EZ, iV, bz, DF, amS, aUN, aDJ, oQ, lC, lf, ne, jr, BA, iu, oO, bM, lD, bkx, if, e, bB, cL, lN, ip, aJO, x, blg, aeO, ja, cvG, xQ, eG, aeR, byj, Jl, fc, hl, Qq, kt, Qm, as, eE, eF, cm, bw, Qt, qJ, bb, xD, acS, go, vA, mx, vy, bru, tG, ym, in, bms, Vj, rt, dD, lT, bMe, n, oY, uE, Cd, EI, md, jz, Xe, gd, eV, bav, cY, A, RT, hT, PX, fJ, hQ, yq, YE, bP, cTF, aOL, hL, at, pg, ut, eo, sZ, B, cB, tq, dA, mr, eX, fF, v, Ge, Cb, V, eJ, Kx, cTH, RI, WY, BJ, rS, nQ, bau, ae, jf, zH, De, vR, vT, ev, blq, Qr, iZ, bSI, bu, t, ho, acp, vk, ck, vW, kP, lI, wJ, gX, hO, yv, biL, hl, ob, Dd, dM, acl, asa, eq, adg, Fc, BY, hd, cTv, py, bNN, Db, Hk, LA, HD, bvM, nJ, sj, d, je, et, Z, arv, bF, gk, yE, bVu, ajw, lI, gZ, Hs, y, aC, UH, fR, al, axk, lR, Vp, cTK, gJ, aGm, xE, cTG, wM, Bc, pX, Ok, M, K, GO, wt, eL, adT, qS, LZ, aB, g, pL, uC, Vm, iP, AM, nF, U, PV, uo, QF, m, P, sQ, sw, Ol, aw, NT, qy, BH, pM, aCQ, cTC, eh, asS, JW, aky, ft, kH, cU, GT, Jn, ZN, Ha, dx, cTE, bsk, cTD, YF, cM, ar, kT, W, afc, bo, JM, bdA, gg, clx, nz, Pr, it, fL, ayV, fM, wj, AL, KR, avm, fG, aGt, jx, zN, j, iI, uW, pd, eA, J, o, er, XX, Mg, anj, aHu, lg, bq, kl, PK, Y, r, en, wA, iK, Dm, Ci, bN, bmS, beJ, Dy, sn, c, hc, bxj, Da, np, dy, EY, D, gT, Kg, bzT, gx, sY, kh, GI, id, Pt, aZ, oA, bS, ah, SY, hF, gb, buj, iJ, aAq, Jm, fU, dP, fX, sN, io, vB, hP, ji, aOh, Yi, uX, aed, BN, lR, anX, vj, eH, cr, si, VI, Fq, iR, Bu, bVw, qa, TA, Bv, Ew, nb, cgA, Fk, CP, ei, vt, G, Tb, auW, pf, eb, zh, aR, bll, cTJ, dQ, MO, eN, hm, DA, ayT, bvR, LC, aW, cnV, eM, iT, uJ, nw, mk, gn, R, Xu, cZ, tp, Wb, ko, fr, ct, bd, qN, ai, eW, Vs, BI, boz, cc, hw, Yc, wG, kl, h, jy, dL, ux, hB, SD, vx, db, Um, wC, CM, iw, aq, kp, mo, gG, dw, op, lx, aoD, oU, bpC, cTz, gY, hk, cTA, Qu, bm, Vy, hR, aSW, uz, aAl, anx, bD, um, bL, eR, atB, dk, yk, rf, cR, tc, cJI, hy, Kh, C, cF, uT, nv, Vr, qp, afQ, HC, aT, dz, Fx, qk, af, qw, Uf, s, iU, aZB, oV, la, aaE, aDm, cTy, f, aDQ, wf, amF, wg, nM, akp, lV, AH, mf, VB, awY, hS, Ke, Zl, qb, aD, CI, Fb, tK, av, F, ot, aaU, Cj, ib, sO, NS, cTB, fb, ul, qt, eZ, Ej, VW, aF, aDd, ek, Hp, LB, es, bPZ, RV, pc, zr, bE, z, uR, Vw, aS, QD, be, XO, GF, tP, X, us); #c1(VEGFA) c2(NP_001165098) c3(vascular endothelial growth factor A isoform m precursor) c4( ) c5(67436, 67437, 67438, 67439, 67440, 67441, 67442, 67443, 67444) c6(67445, 67446) c7(T, bVx, ea, bO, fd, fo, aks, ci, jO, aY, Hr, bv, xy, aE, awe, fm, rw, Vv, hu, aCU, hH, aiJ, cp, aa, ly, yo, iE, wp, bvD, Ls, az, nY, fT, aZx, iA, nB, wE, cj, k, act, bvC, fy, ul, cE, bxC, RX, ht, sh, RC, cQ, aj, PJ, cd, Wy, adL, cty, Cf, bkP, bxi, ey, dd, pl, mR, anP, MD, aJ, Hz, fQ, fe, pm, iO, bkn, dB, bPc, bsO, Be, aoB, E, AN, DD, aA, cz, bX, lu, AO, q, bNX, oT, aWb, vz, MP, qK, cy, jj, sR, rv, dL, eu, le, iF, aGP, NE, nO, fO, bg, bsl, bcD, lo, Lc, cl, bkp, pb, ov, beH, iM, ac, ua, VA, aDR, PN, bt, ao, On, Mb, oq, eP, rQ, iW, uG, jN, jh, dG, uu, fu, xt, ad, hX, afb, aHF, iC, wD, yt, tT, cTl, oW, cTw, lQ, WZ, azH, ue, cH, PE, cW, ff, EH, EZ, iV, bz, DF, amS, aUN, aDJ, oQ, lC, lf, ne, jr, BA, iu, oO, bM, lD, bkx, if, e, bB, cL, IN, ip, aJO, x, blg, aeO, ja, cvG, xQ, eG, aeR, byj, Jl, fc, hl, Qq, kt, Qm, as, eE, eF, cm, bw, Qt, qJ, bb, xD, acS, go, vA, mx, vy, bru, tG, ym, in, bms, Vj, rt, dD, lT, bMe, n, oY, uE, Cd, EI, md, jz, Xe, gd, eV, bav, cY, A, RT, hT, PX, fJ, hQ, yq, YE, bP, cTF, aOL, hl, at, pg, ut, eo, sZ, B, cB, tq, dA, mr, ex, fF, v, Ge, Cb, V, eJ, Kx, cTH, RI, WY, BJ, rS, nd, bau, ae, jf, zH, De, vR, vT, ev, blq, Or, iZ, bSI, bu, t, ho, acp, vk, ck, vW, kP, lI, wl, gX, hO, yv, biL, hl, ob, Dd, dM, acl, asa, eq, adg, Fc, BY, hd, cTv, py, bNN, Db, Hk, LA, HD, bvM, nJ, sj, d, je, et, Z, arv, bF, gk, yE, bVu, ajw, lI, gZ, Hs, y, aC, UH, FR, al, axk, lR, Vp, cTK, gJ, aGm, xE, cTG, wM, Bc, pX, Qk, M, K, GO, wt, eL, adT, qS, LZ, aB, g, pL, uC, Vm, iP, AM, nF, U, PV, uo, QF, m, P, sQ, sw, Ol, aw, NT, qy, BH, pM, aCQ, cTC, eh, asS, JW, aky, ft, kH, cU, GT, Jn, ZN, Ha, dx, cTE, bsK, cTD, YF, cM, ar, kT, W, afc, bo, JM, bdA, gg, cTx, nz, Pr, it, fL, ayV, fM, wj, AL, KR, avm, fG, aGt, jx, zN, j, iI, uW, pd, eA, J, o, er, XX, Mg, anj, aHu, lg, bq, kl, PK, Y, r, en, wA, ik, Dm, Ci, bN, bmS, beJ, Dy, sn, c, hc, bxj, Da, np, dy, EY, fD, gT, Kg, bzT, gx, sY, kh, GI, id, Pt, aZ, oA, bS, ah, SY, hF, gb, buj, iJ, aAq, Jm, fU, dP, fX, sN, io, vB, hP, ji, aOh, Yi, uX, ael, BN, lR, anX, vj, eH, cr, si, VI, Fq, iR, Bu, bVw, qa, TA, Bv, Ew, nb, cgA, Fk, cP, ei, vt, G, Tb, auW, pf, eb, zh, aR, bll, cTJ, dQ, MO, eN, hm, DA, ayT, bvR, LC, aW, cnV, eM, iT, uJ, nw, mk, gn, R, Xu, cZ, tp, Wb, ko, fr, ct, bd, qN, ai, eW, Vs, BI, boz, cc, hw, Yc, wG, kl, h, jy, dL, ux, HB, SD, vx, db, Um, WC, CM, iw, aq, kp, mo, gG, dw, op, lx, aoO, ou, bpC, cTz, gY, hk, cTA, Qu, bm, Vy, hR, aSW, uz, aAl, anx, bD, um, bl, eR, atB, dK, yk, rf, cR, tc, cJI, hy, Kh, C, cF, uT, nv, Vr, qp, afQ, HC, aT, dz, Fx, qk, af, qw, Uf, s, iI, aZB, oV, la, aaE, aDm, cTy, f, aDQ, wf, amF, wg, nM, akp, lV, AH, mf, VB, awY, hS, Ke, Zl, qb, aD, CI, Fb, tK, av, F, ot, aaU, Cj, ib, sO, NS, cTB, fb, ul, qt, eL, Ej, VW, aF, aDd, ek, Hp, LB, es, bPZ, RV, pc, zr, bE, z, uR, Vw, aS, QD, be, XO, GF, tP, X, us); #c1(VEGFA) c2(NP_001165099) c3 (vascular endothelial growth factor A isoform n precursor) c4( ) c5(67447, 67448, 67449, 67450, 67451, 67452, 67453, 67454) c6(67455, 67456) c7(T, bVx, ea, bO, fd, fo, aks, ci, jO, aY, Hr, bv, xy, aE, awe, fm, rw, Vv, hu, aCU, hH, aiJ, cp, aa, ly, yo, iE, wp, bvD, Ls, az, nY, fT, aZx, iA, nB, wE, cj, k, act, bvC, fy, ul, cE, bxC, RX, ht, sh, RC, cQ, aj, PJ, cd, Wy, adL, cty, Cf, bkP, bxi, ey, dd, pl, mR, anP, MD, aJ, Hz, fQ, fe, pm, iO, bkn, dB, bPc, bsO, Be, aoB, E, AN, DD, aA, cz, bX, lu, AO, q, bNX, OT, aWb, vz, MP, qK, cy, jj, sR, rv, dL, eu, le, if, aGP, NE, nO, fO, bg, bsl, bcD, lo, Lc, cl, bkp, pb, ov, beH, iM, ac, ua, VA, aDR, PN, bt, ao, On, Mb, oq, eP, rQ, iW, uG, jN, jh, dG, uu, fu, xt, ad, hX, afb, aHF, iC, wD, yt, tT, cTl, oW, cTw, lQ, WZ, azH, ue, CH, PE, cW, ff, EH, EZ, iV, bz, DF, amS, aUN, aDJ, oQ, lC, lf, ne, jr, BA, iu, oO, bM, lD, bkx, if, e, bB, cL, lN, ip, aJO, x, blg, aeO, ja, cvG, xQ, eG, aeR, byj, Jl, fc, hl, Qq, kt, Qm, as, eE, eF, cm, bw, Qt, qJ, bb, xD, acS, go, vA, mx, vy, bru, tG, ym, in, bms, Vj, rt, dD, lT, bMe, n, oY, uE, Cd, EI, md, jz, Xe, gd, eV, bav, cY, A, RT, hT, PX, fJ, hQ, yq, YE, bP, cTF, aOL, hl, at, pg, ut, eo, sZ, B, cB, tq, dA, mr, eX, fF, v, Ge, Cb, V, eJ, Kx, cTH, RI, WY, BJ, rS, nQ, bau, ae, jf, zH, De, vR, vT, ev, blq, Qr, iZ, bSI, bu, t, ho, acp, vk, ck, vW, kP, lI, wD, gX, hO, yv, biL, hl, ob, Dd, dM, acl, asa, eq, adg, Fc, BY, hd, cTv, py, bNN, Db, Hk, LA, hD, bvM, nJ, sj, d, je, et, Z, arv, bF, gk, yE, bVu, ajw, lI, gZ, Hs, y, aC, UH, fR, al, axk, lR, Vp, cTK, gJ, aGm, xE, cTG, wM, Bc, pX, Qk, M, K, GO, wt, eL, adT, qS, LZ, aB, g, pL, uC, Vm, iP, AM, nF, U, PV, uo, QF, m, P, sl, sw, OI, aw, NT, qy, BH, pM, aCQ, cTC, eh, asS, JW, aky, ft, kH, cU, GT, Jn, ZN, Ha, dx, cTE, bsk, cTD, YF, cM, ar, kT, W, afc, bo, JM, bdA, gg, cTx, nz, Pr, it, fL, ayV, fM, wj, AL, KR, avm, fG, aGt, jx, zN, j, iI, uW, pd, eA, J, o, er, XX, Mg, anj, aHu, lg, bq, kl, PK, Y, r, en, wA, iK, Dm, Ci, bN, bmS, beJ, Dy, sn, c, hc, bxj, Da, np, dy, EY, fD, gT, Kg, bzT, gx, sY, kh, GI, id, Pt, aZ, oA, bS, ah, SY, hF, gb, buj, iJ, aAq, Jm, fU, dP, fX, sN, io, vB, hP, ji, aOh, Yi, uX, aed, BN, lR, anX, vj, eH, cr, si, VI, Fq, iR, Bu, bVw, qa, TA, Bv, Ew, nb, cgA, Fk, cP, ei, vt, G, Tb, auW, pf, eb, zh, aR, bll, cTJ, dL, MO, eN, hm, DA, ayT, bvR, LC, aW, cnV, eM, iT, uJ, nw, mk, gn, R, Xu, cZ, tp, Wb, ko, fr, ct, bd, qN, ai, eW, Vs, BI, boz, cc, hw, Yc, wG, kl, h, jy, dL, ux, hB, SD, vx, db, Um, wC, CM, iw, aq, kp, mo, gG, dw, op, lx, aoD, oU, bpC, cTz, gY, hk, cTA, Qu, bm, Vy, hR, aSW, uz, aAl, anx, bD, um, bL, eR, atB, dK, yk, rf, cR, tc, cJl, hy, Kh, C, cF, uT, nv, Vr, qp, afQ, HC, aT, dz, Fx, qk, af, qw, Uf, s, iU, aZB, oV, la, aaE, aDm, cTy, f, aDQ, wf, amF, wg, nM, akp, lV, AH, mf, VB, awY, hS, Ke, Zl, qb, aD, CI, Fb, tK, av, F, ot, aaU, Cj, ib, sO, NS, cTB, fb, ul, qt, eZ, Ej, VW, aF, aDd, ek, Hp, LB, es, bPZ, RV, pc, zr, bE, z, uR, Vw, aS, QD, be, XO, GF, tP, X, us); #c1(VEGFA) c2(NP_001165100) c3(vascular endothelial growth factor A isoform o precursor) c4( ) c5(67457, 67458, 67459, 67460, 67461, 67462, 67463, 67464, 67465) c6(67466, 67467) c7(T, bVx, ea, bO, fd, fo, aks, ci, jO, aY, Hr, bv, xy, aE, awe, fm, rw, Vv, hu, aCU, hH, aiJ, cp, aa, ly, yo, iE, wp, bvD, Ls, az, nY, fT, aZx, iA, nB, wE, cj, k, act, bvC, fy, ul, cE, bxC, RX, ht, sh, RC, cd, aj, PJ, cd, Wy, adL, cty, Cf, bkP, bxi, ey, dd, pl, mR, anP, MD, aJ, Hz, fQ, fe, pm, iO, bkn, dB, bPc, bsO, Be, aoB, E, AN, DD, aA, cz, bX, lu, AO, q, bNX, oT, aWb, vz, MP, qK, cy, jj, sR, rv, dL, eu, le, if, aGP, NE, nO, fO, bg, bsl, bcD, lo, Lc, cl, bkp, pb, ov, beH, iM, ac, ua, VA, aDR, PN, bt, ao, On, Mb, oq, eP, rQ, iW, uG, jN, jh, dG, uu, fu, xt, ad, hX, afb, aHF, iC, wD, yt, tT, cTl, oW, cTw, lQ, WZ, azH, ue, cH, PE, cW, ff, EH, EZ, iV, bz, DF, amS, aUN, aDJ, oQ, lC, lf, ne, jr, BA, iu, oO, bM, lD, bkx, if, e, bB, cL, lN, ip, aJO, x, blg, aeO, ja, cvG, xQ, eG, aeR, byj, Jl, fc, hl, Qq, kt, Qm, as, eE, eF, cm, bw, Qt, qJ, bb, xD, acS, go, vA, mx, vy, bru, tG, ym, in, bms, Vj, rt, dD, lT, bMe, n, oY, uE, Cd, EI, md, jz, Xe, gd, eV, bav, cY, A, RT, hT, PX, fJ, hQ, yq, YE, bP, cTF, aOL, hl, at, pg, ut, eo, sZ, B, cB, tq, dA, mr, eX, fF, v, Ge, Cb, V, eJ, Kx, cTH, RI, WY, BJ, rS, n, bau, ae, jf, zH, De, vR, vT, ev, blq, Qr, iZ, bSI, bu, t, ho, acp, vk, ck, vW, kP, lI, wl, gX, hO, yv, biL, hl, ob, Dd, dM, acl, asa, eq, adg, Fc, BY, hd, cTv, py, bNN, Db, Hk, LA, HD, bvM, nJ, sj, d, je, et, Z, arv, bF, gk, yE, bVu, ajw, lI, gZ, Hs, y, aC, UH, fR, al, axk, lR, Vp, cTK, gJ, aGm, xE, cTG, wM, Bc, pX, Qk, M, K, GO, wt, eL, adT, qS, LZ, aB, g, pL, uC, Vm, iP, AM, nF, U, PV, uo, QF, m, P, sQ, sw, Ol, aw, NT, qy, BH, pM, aCQ, cTC, eh, asS, JW, aky, ft, kH, cU, GT, Jn, ZN, Ha, dx, cTE, bsk, cTD, YF, cM, ar, KT, W, afc, bo, JM, bdA, gg, cTx, nz, Pr, it, fL, ayV, fM, wj, AL, KR, avm, fG, aGt, jx, zN, j, iI, uW, pd, eA, J, o, er, XX, Mg, anj, aHu, lg, bq, kl, PK, Y, r, en, wA, iK, Dm, Ci, bN, bmS, beJ, Dy, sn, c, hc, bxj, Da, np, dy, EY, fD, gT, Kg, bzT, gx, sY, kh, GI, id, Pt, aZ, oA, bS, ah, SY, hF, gb, buj, iJ, aAq, Jm, fU, dP, fX, sN, io, vB, hP, ji, aOh, Yi, uX, aed, BN, lR, anX, vj, eH, cr, si, VI, Fq, iR, Bu, bVw, qa, TA, Bv, Ew, nb, cgA, Fk, cP, ei, vt, G, Tb, auW, pf, eb, zh, aR, bll, cTJ, dQ, MO, eN, hm, DA, ayT, bvR, LC, aW, cnV, eM, iT, uJ, nw, mk, gn, R, Xu, cZ, tp, Wb, ko, fr, ct, bd, qN, ai, eW, Vs, BI, boz, cc, hw, Yc, wG, kl, h, jy, dL, ux, hB, SD, vx, db, Um, WC, CM, iw, aq, kp, mo, gG, dw, op, lx, aoD, oU, bpC, cTz, gY, hk, cTA, Qu, bm, Vy, hR, aSW, uz, aAl, anx, bD, um, bL, eR, atB, dk, yk, rf, cR, tc, cJl, hy, Kh, C, cF, uT, nv, Vr, qp, afQ, HC, aT, dz, Fx, qk, af, qw, Uf, s, iI, aZB, oV, la, aaE, aDm, cTy, f, aDQ, wf, amF, wg, nM, akp, lV, AH, mf, VB, awY, hS, Ke, Zl, qb, aD, CI, Fb, tK, av, F, ot, aaU, Cj, ib, sO, NS, cTB, fb, ul, qt, eZ, Ej, VW, aF, aDd, ek, Hp, LB, es, bPZ, RV, pc, zr, bE, z, uR, Vw, aS, QD, be, XO, GF, tP, X, us); #c1(VEGFA) c2(NP_001165101) c3(vascular endothelial growth factor A isoform p precursor) c4( ) c5(67468, 67469, 67470, 67471, 67472, 67473, 67474, 67475, 67476) c6(67477, 67478) c7(T, bVx, ea, bO, fd, fo, aks, ci, jO, aY, Hr, bv, xy, aE, awe, fm, rw, Vv, hu, aCU, hH, aiJ, cp, aa, ly, yo, iE, wp, bvD, Ls, az, nY, fT, aZx, iA, nB, wE, cj, k, act, bvC, fy, ul, cE, bxC, RX, ht, sh, RC, cQ, aj, PJ, cd, Wy, adL, cty, Cf, bkP, bxi, ey, dd, pl, mR, anP, MD, aJ, Hz, fQ, fe, pm, iO, bkn, dB, bPc, bsO, Be, aoB, E, AN, DD, aA, cz, bX, lu, AD, q, bNX, oT, aWb, vz, MP, qK, cy, jj, sR, rv, dL, eu, le, iF, aGP, NE, nO, fO, bg, bsl, bcD, lo, Lc, cl, bkp, pb, ov, beH, iM, ac, ua, VA, aDR, PN, bt, ao, On, Mb, oq, eP, rQ, iW, uG, jN, jh, dG, uu, fu, xt, ad, hX, afb, aHF, iC, wD, yt, tT, cTl, oW, cTw, lQ, WZ, azH, ue, CH, PE, cW, ff, EH, EZ, iV, bz, DF, amS, aUN, aDJ, oQ, lC, lf, ne, jr, BA, iu, oO, bM, lD, bkx, if, e, bB, cL, lN, ip, aJO, x, blg, aeO, ja, cvG, xQ, eG, aeR, byj, Jl, fc, hl, Qq, kt, Qm, as, eE, eF, cm, bw, Qt, qJ, bb, xD, acS, go, vA, mx, vy, bru, tG, ym, in, bms, Vj, rt, dD, lT, bMe, n, oY, uE, Cd, EI, md, jz, Xe, gd, eV, bav, cY, A, RT, hT, PX, fJ, hQ, yq, YE, bP, cTF, aOL, hl, at, pg, ut, eo, sZ, B, cB, tq, dA, mr, eX, fF, v, Ge, Cb, V, eJ, Kx, cTH, RI, WY, BJ, rS, nQ, bau, ae, jf, zH, De, vR, vT, ev, blq, Qr, iZ, bSI, bu, t, ho, acp, vk, ck, vW, kP, lI, wl, gX, hO, yv, biL, hl, ob, Dd, dM, acl, asa, eq, adg, Fc, BY, hd, cTv, py, bNN, Db, Hk, LA, hD, bvM, nJ, sj, d, je, et, Z, arv, bF, gk, yE, bVu, ajw, lI, gZ, Hs, y, aC, UH, fR, al, axk, lR, Vp, cTK, gJ, aGm, xE, CTG, wM, Bc, pX, Qk, M, K, GO, wt, eL, adT, qS, LZ, aB, g, pL, uC, Vm, iP, AM, nF, U, PV, uo, QF, m, P, sQ, sw, OI, aw, NT, qy, BH, pM, aCQ, cTC, eh, asS, JW, aky, ft, kH, cU, GT, Jn, ZN, Ha, dx, cTE, bsk, cTD, YF, cM, ar, kT, W, afc, bo, JM, bdA, gg, cTx, nz, Pr, it, fL, ayV, fM, wj, AL, KR, avm, fG, aGt, jx, zN, j, iI, uW, pd, eA, J, o, er, XX, Mg, anj, aHu, lg, bq, kl, PK, Y, r, en, wA, iK, Dm, Ci, bN, bmS, beJ, Dy, sn, c, hc, bxj, Da, np, dy, EY, fD, gT, Kg, bzT, gx, sY, kh, GI, id, Pt, aZ, oA, bS, ah, SY, hF, gb, buj, iJ, aAq, Jm, fU, dP, fX, sN, io, vB, hP, ji, ah, Yi, uX, aeQ, BN, lR, anX, vj, eH, cr, si, VI, Fq, iR, Bu, bVw, qa, TA, Bv, Ew, nb, cgA, Fk, cP, ei, vt, G, Tb, auW, pf, eb, zh, aR, bll, cTJ, dQ, MO, eN, hm, DA, ayT, bvR, LC, aW, cnV, eM, iT, uJ, nw, mk, gn, R, Xu, cZ, tp, Wb, ko, fr, ct, bd, qN, ai, eW, Vs, BI, boz, cc, hw, Yc, wG, kl, h, jy, dL, ux, hB, SD, vx, db, Um, wC, CM, iw, aq, kp, mo, gG, dw, op, lx, aoO, oU, bpC, cTz, gY, hk, cTA, Qu, bm, Vy, hR, aSW, uz, aAl, anx, bD, um, bL, eR, atB, dK, yk, rf, cR, tc, cJl, hy, Kh, C, cF, uT, nv, Vr, qp, afQ, HC, aT, dz, Fx, qk, af, qw, Uf, s, iI, aZB, oV, la, aaE, aDm, cTy, f, aDQ, wf, amF, wg, nM, akp, lV, AH, mf, VB, awY, hS, Ke, Zl, qb, aD, CI, Fb, tK, av, F, ot, aaU, Cj, ib, sO, NS, cTB, fb, ud, qt, eZ, Ej, VW, aF, aDd, eK, Hp, LB, es, bPZ, RV, pc, zr, bE, z, uR, Vw, aS, QD, be, XO, GF, tP, X, us); #c1(VEGFA) c2(NP_001191314) c3(vascular endothelial growth factor A isoform r) c4(81365, 81366, 81367) c5(81368, 81369, 81370, 81371, 81372, 81373, 81374, 81375, 81376, 81377) c6(81378, 81379) c7(T, bVx, ea, bO, fd, fo, aks, ci, jO, aY, Hr, bv, xy, aE, awe, fm, rw, Vv, hu, aCU, hH, aiJ, cp, aa, ly, yo, iE, wp, bvD, Ls, az, nY, fT, aZx, iA, nB, wE, cj, k, act, bvC, fy, ul, CE, bxC, RX, ht, sh, RC, cQ, aj, PJ, cd, Wy, adL, cty, Cf, bkP, bxi, ey, dd, pl, mR, anP, MD, aJ, Hz, fQ, fe, pm, iO, bkn, dB, bPc, bsO, Be, aoB, E, AN, DD, aA, cz, bX, lu, AD, q, bNX, oT, aWb, vz, MP, qK, cy, jj, sR, rv, dL, eu, le, iF, aGP, NE, nO, fO, bg, bsl, bcD, lo, Lc, cl, bkp, pb, ov, beH, iM, ac, ua, VA, aDR, PN, bt, ao, On, Mb, oq, eP, rQ, iW, uG, jN, jh, dG, uu, fu, xt, ad, hX, afb, aHF, iC, wD, yt, tT, cTl, oW, cTw, lQ, WZ, azH, ue, cH, PE, cW, ff, EH, EZ, iV, bz, DF, amS, aUN, aDJ, od, lC, lf, ne, jr, BA, iu, oO, bM, lD, bkx, if, e, bB, cL, lN, ip, aJO, x, blg, aeO, ja, cvG, xQ, eG, aeR, byj, Jl, fc, hl, Qq, kt, Am, as, eE, eF, cm, bw, Qt, qJ, bb, xD, acS, go, vA, mx, vy, bru, tG, ym, in, bms, Vj, rt, dD, lT, bMe, n, oY, uE, Cd, EI, md, jz, Xe, gd, eV, bav, cY, A, RT, hT, PX, fJ, hQ, yq, YE, bP, cTF, aOL, hl, at, pg, ut, eo, sZ, B, cB, tq, dA, mr, eX, fF, v, Ge, Cb, V, eJ, Kx, cTH, RI, WY, BJ, rS, nQ, bau, ae, jf, zH, De, vR, vT, ev, blq, Ar, iZ, bSI, bu, t, ho, acp, vk, ck, vW, kP, lI, wD, gX, hO, yv, biL, hl, ob, Dd, dM, acl, asa, eq, adg, Fc, BY, hd, cTv, py, bNN, Db, Hk, LA, HD, bvM, nJ, sj, d, je, et, Z, arv, bF, gk, yE, bVu, ajw, lI, gZ, Hs, y, aC, UH, fR, al, axk, lR, Vp, cTK, gJ, aGm, xE, CTG, wM, Bc, pX, Qk, M, K, GO, wt, eL, adT, qS, LZ, aB, g, pL, uC, Vm, iP, AM, nF, U, PV, uo, QF, m, P, sQ, sw, Ol, aw, NT, qy, BH, PM, aCQ, cTC, eh, asS, JW, aky, ft, kH, cU, GT, Jn, ZN, Ha, dx, cTE, bsk, cTD, YF, cM, ar, kT, W, afc, bo, JM, bdA, gg, cTx, nz, Pr, it, fL, ayV, fM, wj, AL, KR, avm, fG, aGt, jx, zN, j, iI, uW, pd, eA, J, o, er, XX, Mg, anj, aHu, lg, bq, kl, PK, Y, r, en, wA, iK, Dm, Ci, bN, bmS, beJ, Dy, sn, c, hc, bxj, Da, np, dy, EY, fD, gT, Kg, bzT, gx, sY, kh, GI, id, Pt, aZ, oA, bS, ah, SY, hF, gb, buj, iJ, aAq, Jm, fU, dP, fX, sN, io, vB, hP, ji, aOh, Yi, uX, aeQ, BN, lR, anX, vj, eH, cr, si, VI, Fq, iR, Bu, bVw, qa, TA, Bv, Ew, nb, cgA, Fk, cP, ei, vt, G, Tb, auW, pf, eb, zh, aR, bll, cTJ, dQ, MO, eN, hm, DA, ayT, bvR, LC, aW, cnV, eM, iT, uJ, nw, mk, gn, R, Xu, cZ, tp, Wb, ko, fr, ct, bd, qN, ai, eW, Vs, BI, boz, cc, hw, Yc, wG, kl, h, jy, dL, ux, hB, SD, vx, db, Um, wC, CM, iw, aq, kp, mo, gG, dw, op, lx, aoO, oU, bpC, cTz, gY, hk, cTA, Qu, bm, Vy, hR, aSW, uz, aAl, anx, bD, um, bL, eR, atB, dK, yk, rf, cR, tc, cJl, hy, Kh, C, cF, uT, nv, Vr, qp, afQ, HC, aT, dz, Fx, qk, af, qw, Uf, s, iI, aZB, oV, la, aaE, aDm, cTy, f, aDQ, wf, amF, wg, nM, akp, lV, AH, mf, VB, awY, hS, Ke, Zl, qb, aD, CI, Fb, tK, av, F, ot, aaU, Cj, ib, sO, NS, cTB, fb, uQ, qt, eZ, Ej, VW, aF, aDd, ek, Hp, LB, es, bPZ, RV, pc, zr, bE, z, uR, Vw, aS, QD, be, XO, GF, tP, X, us); #c1(VEGFA) c2(NP_001273973) c3(vascular endothelial growth factor A isoform s) c4( ) c5(105944, 105945, 105946, 105947, 105948, 105949, 105950, 105951) c6( ) c7(T, bVx, ea, bO, fd, fo, aks, ci, jO, aY, Hr, bv, xy, aE, awe, fm, rw, Vv, hu, aCU, hH, aiJ, cp, aa, ly, yo, iE, wp, bvD, Ls, az, nY, fT, aZx, iA, nB, wE, cj, k, act, bvC, fy, ul, CE, bxC, RX, ht, sh, RC, cQ, aj, PJ, cd, Wy, adL, cty, Cf, bkP, bxi, ey, dd, pl, mR, anP, MD, aJ, Hz, fQ, fe, pm, iO, bkn, dB, bPc, bsO, Be, aoB, E, AN, DD, aA, cz, bX, lu, AO, q, bNX, oT, aWb, vz, MP, qK, cy, jj, sR, rv, dL, eu, le, if, aGP, NE, nO, fO, bg, bsl, bcD, lo, Lc, cl, bkp, pb, ov, beH, iM, ac, ua, VA, aDR, PN, bt, ao, On, Mb, oq, eP, rl, iW, uG, jN, jh, dG, uu, fu, xt, ad, hX, afb, aHF, iC, wD, yt, tT, cTl, oW, cTw, lQ, WZ, azH, ue, CH, PE, cW, ff, EH, EZ, iV, bz, DF, amS, aUN, aDJ, ol, IC, lf, ne, jr, BA, iu, oO, bM, lD, bkx, if, e, bB, cL, lN, ip, aJO, x, blg, aeO, ja, cvG, xQ, eG, aeR, byj, Jl, fc, hl, Qq, kt, Qm, as, eE, eF, cm, bw, lt, qJ, bb, xD, acS, go, vA, mx, vy, bru, tG, ym, in, bms, Vj, rt, dD, lT, bMe, n, oY, uE, Cd, EI, md, jz, Xe, gd, eV, bav, cY, A, RT, hT, PX, fJ, hQ, yq, YE, bP, cTF, aOL, hL, at, pg, ut, eo, sZ, B, cB, tq, dA, mr, eX, fF, v, Ge, Cb, V, eJ, Kx, cTH, RI, WY, BJ, rS, nd, bau, ae, jf, zH, De, vR, vT, ev, blq, Ar, iZ, bSI, bu, t, ho, acp, vk, ck, vW, kP, lI, wl, gX, hO, yv, biL, hl, ob, Dd, dM, acl, asa, eq, adg, Fc, BY, hd, cTv, py, bNN, Db, Hk, LA, HD, bvM, nJ, sj, d, je, et, Z, arv, bF, gk, yE, bVu, ajw, lI, gZ, Hs, y, aC, UH, fR, al, axk, lR, Vp, cTK, gJ, aGm, xE, cTG, wM, Bc, pX, Qk, M, K, GO, wt, eL, adT, qS, LZ, aB, g, pL, uC, Vm, iP, AM, nF, U, PV, uo, QF, m, P, sQ, sw, Ol, aw, NT, qy, BH, pM, aCQ, cTC, eh, asS, JW, aky, ft, kH, cU, GT, Jn, ZN, Ha, dx, cTE, bsk, cTD, YF, cM, ar, kT, W, afc, bo, JM, bdA, gg, cTx, nz, Pr, it, fL, ayV, fM, wj, AL, KR, avm, fG, aGt, jx, zN, j, iI, uW, pd, eA, J, o, er, XX, Mg, anj, aHu, lg, bq, kl, PK, Y, r, en, wA, iK, Dm, Ci, bN, bmS, beJ, Dy, sn, c, hc, bxj, Da, np, dy, EY, fD, gT, Kg, bzT, gx, sY, kh, GI, id, Pt, aZ, oA, bS, ah, SY, hF, gb, buj, iJ, aAq, Jm, fU, dP, fX, sN, io, vB, hP, ji, aOh, Yi, uX, aed, BN, lR, anX, vj, eH, cr, si, VI, Fq, iR, Bu, bVw, qa, TA, Bv, Ew, nb, cgA, Fk, cP, ei, vt, G, Tb, auW, pf, eb, zh, aR, bll, cTJ, de, MO, eN, hm, DA, ayT, bvR, LC, aW, cnV, eM, iT, uJ, nw, mk, gn, R, Xu, cZ, tp, Wb, ko, fr, ct, bd, qN, ai, eW, Vs, BI, boz, cc, hw, Yc, wG, kl, h, jy, dL, ux, hB, SD, vx, db, Um, wC, CM, iw, aq, kp, mo, gG, dw, op, lx, aoO, oU, bpC, cTz, gY, hk, cTA, Qu, bm, Vy, hR, aSW, uz, aAl, anx, bD, um, bL, eR, atB, dk, yk, rf, cR, tc, cJl, hy, Kh, C, cF, uT, nv, Vr, qp, afQ, HC, aT, dz, Fx, qk, af, qw, Uf, s, iI, aZB, oV, la, aaE, aDm, cTy, f, aDQ, wf, amF, wg, nM, akp, lV, AH, mf, VB, awY, hS, Ke, Zl, qb, aD, CI, Fb, tK, av, F, ot, aaU, Cj, ib, sO, NS, cTB, fb, ud, qt, eZ, Ej, VW, aF, aDd, ek, Hp, LB, es, bPZ, RV, pc, zr, bE, z, uR, Vw, aS, QD, be, XO, GF, tP, X, us); #c1(VEGFA) c2(NP_003367) c3(vascular endothelial growth factor A isoform b) c4(137823, 137824, 137825) c5(137826, 137827, 137828, 137829, 137830, 137831, 137832, 137833, 137834, 137835) c6(137836, 137837) c7(T, bVx, ea, bO, fd, fo, aks, ci, jO, aY, Hr, bv, xy, aE, awe, fm, rw, Vv, hu, aCU, hH, aiJ, cp, aa, ly, yo, iE, wp, bvD, Ls, az, nY, fT, aZx, iA, nB, wE, cj, k, act, bvC, fy, ul, cE, bxC, RX, ht, sh, RC, cQ, aj, PJ, cd, Wy, adL, cty, Cf, bkP, bxi, ey, dd, pl, mR, anP, MD, aJ, Hz, fQ, fe, pm, iO, bkn, dB, bPc, bsO, Be, aoB, E, AN, DD, aA, cz, bX, lu, AO, q, bNX, oT, aWb, vz, MP, qK, cy, jj, sR, rv, dL, eu, le, if, aGP, NE, nO, fO, bg, bsl, bcD, lo, Lc, cl, bkp, pb, ov, beH, iM, ac, ua, VA, aDR, PN, bt, ao, On, Mb, oq, eP, rQ, iW, uG, jN, jh, dG, uu, fu, xt, ad, hX, afb, aHF, iC, wD, yt, tT, cTl, oW, cTw, lQ, WZ, azH, ue, CH, PE, cW, ff, EH, EZ, iV, bz, DF, amS, aUN, aDJ, od, lC, lf, ne, jr, BA, iu, oO, bM, lD, bkx, if, e, bB, cL, lN, ip, aJO, x, blg, aeO, ja, cvG, xQ, eG, aeR, byj, Jl, fc, hl, Qq, kt, Qm, as, eE, eF, cm, bw, Qt, qJ, bb, xD, acS, go, vA, mx, vy, bru, tG, ym, in, bms, Vj, rt, dD, lT, bMe, n, oY, uE, Cd, EI, md, jz, Xe, gd, eV, bav, cY, A, RT, hT, PX, fJ, hQ, yq, YE, bP, cTF, aOL, hL, at, pg, ut, eo, sZ, B, cB, tq, dA, mr, eX, fF, v, Ge, Cb, V, eJ, Kx, cTH, RI, WY, BJ, rS, nd, bau, ae, jf, zH, De, vR, vT, ev, blq, Qr, iZ, bSI, bu, t, ho, acp, vk, ck, vW, kP, lI, wD, gX, hO, yv, biL, hl, ob, Dd, dM, acl, asa, eq, adg, Fc, BY, hd, cTv, py, bNN, Db, Hk, LA, hD, bvM, nJ, sj, d, je, et, Z, arv, bF, gk, yE, bVu, ajw, lI, gZ, Hs, y, aC, UH, fR, al, axk, lR, Vp, cTK, gJ, aGm, xE, cTG, wM, Bc, pX, Qk, M, K, GO, wt, eL, adT, qS, LZ, aB, g, pL, uC, Vm, iP, AM, nF, U, PV, uo, QF, m, P, sQ, sw, OI, aw, NT, qy, BH, pM, aCQ, cTC, eh, asS, JW, aky, ft, kH, cU, GT, Jn, ZN, Ha, dx, cTE, bsK, cTD, YF, cM, ar, kT, W, afc, bo, JM, bdA, gg, cTx, nz, Pr, it, fL, ayV, fM, wj, AL, KR, avm, fG, aGt, jx, zN, j, iI, uW, pd, eA, J, o, er, XX, Mg, anj, aHu, lg, bq, kl, PK, Y, r, en, wA, iK, Dm, Ci, bN, bmS, beJ, Dy, sn, c, hc, bxj, Da, np, dy, EY, fD, gT, Kg, bzT, gx, sY, kh, GI, id, Pt, aZ, oA, bS, ah, SY, hF, gb, buj, iJ, aAq, Jm, fU, dP, fX, sN, io, vB, hP, ji, aOh, Yi, uX, ael, BN, lR, anX, vj, eH, cr, si, VI, Fq, iR, Bu, bVw, qa, TA, Bv, Ew, nb, cgA, Fk, cP, ei, vt, G, Tb, auW, pf, eb, zh, aR, bll, cTJ, dQ, MO, eN, hm, DA, ayT, bvR, LC, aW, cnV, eM, iT, uJ, nw, mk, gn, R, Xu, cZ, tp, Wb, ko, fr, ct, bd, qN, ai, eW, Vs, BI, boz, cc, hw, Yc, wG, kl, h, jy, dL, ux, hB, SD, vx, db, Um, wC, CM, iw, aq, kp, mo, gG, dw, op, lx, aoO, oU, bpC, cTz, gY, hk, cTA, Qu, bm, Vy, hR, aSW, uz, aAl, anx, bD, um, bL, eR, atB, dK, yk, rf, cR, tc, cJl, hy, Kh, C, cF, uT, nv, Vr, qp, afQ, HC, aT, dz, Fx, qk, af, qw, Uf, s, iI, aZB, oV, la, aaE, aDm, cTy, f, aDQ, wf, amF, wg, nM, akp, lV, AH, mf, VB, awY, hS, Ke, Zl, qb, aD, CI, Fb, tK, av, F, ot, aaU, Cj, ib, sO, NS, cTB, fb, ul, qt, eZ, Ej, VW, aF, aDd, ek, Hp, LB, es, bPZ, RV, pc, zr, bE, z, uR, Vw, aS, QD, be, XO, GF, tP, X, us); #c1(VEGFB) c2(NP_001230662) c3(vascular endothelial growth factor B isoform VEGFB-167 precursor) c4(86526) c5(86527, 86528, 86529, 86530, 86531, 86532, 86533, 86534) c6( ) c7(sY, T, dD, aw, j, ci, eq, fr, cd, aR, ad, cTL, ip, aE, Db, Vv, cy, adx, s, as, cl, uC, iA, U, hL, gZ, m, B); #c1(VEGFB) c2(NP_003368) c3(vascular endothelial growth factor B isoform VEGFB-186 precursor) c4(137838) c5(137839, 137840, 137841, 137842, 137843, 137844, 137845) c6( ) c7(sY, T, dD, aw, j, ci, eq, fr, cd, aR, ad, cTL, ip, aE, Db, Vv, cy, adx, s, as, cl, uC, iA, U, hL, gZ, m, B); #c1(VEGFC) c2(NP_005420) c3(vascular endothelial growth factor C preproprotein) c4(155885, 155886) c5(155887, 155888, 155889, 155890, 155891, 155892, 155893, 155894, 155895, 155896, 155897, 155898, 155899, 155900, 155901, 155902, 155903, 155904, 155905, 155906, 155907, 155908, 155909, 155910, 155911, 155912, 155913, 155914, 155915, 155916, 155917, 155918, 155919, 155920, 155921, 155922, 155923, 155924, 155925, 155926, 155927, 155928, 155929, 155930, 155931, 155932, 155933, 155934, 155935, 155936, 155937, 155938, 155939, 155940, 155941, 155942, 155943, 155944, 155945, 155946, 155947, 155948, 155949, 155950, 155951, 155952, 155953, 155954, 155955, 155956, 155957, 155958, 155959, 155960, 155961, 155962, 155963, 155964, 155965, 155966, 155967, 155968, 155969, 155970, 155971, 155972, 155973, 155974, 155975, 155976, 155977, 155978, 155979, 155980) c6( ) c7(uB, T, aC, Tb, if, eb, bB, ci, PB, aGm, aE, Vv, K, hu, eL, cp, aa, dH, LC, aW, g, eE, eF, uC, cm, bw, qJ, eM, nB, iA, vy, PO, m, ym, Vj, iu, oO, sw, ht, n, aj, fr, qN, ft, jz, h, lG, dL, hB, hi, mR, hl, kp, fq, W, Vy, hR, it, B, fM, dA, AL, aA, j, Cb, q, J, eA, nv, aT, cy, ae, s, xR, oV, Y, r, en, t, iK, ck, bmS, nM, Dy, gX, PN, bt, hS, Mb, sY, acl, iW, jh, dG, gb, ad, Jm, dP, av, hd, ib, Db, ds, io, d, ff, aF, bPK, bz, bE, y, X); #c1(VENTX) c2(NP_055283) c3(homeobox protein VENTX) c4(180267) c5(180268, 180269, 180270, 180271, 180272, 180273, 180274) c6( ) c7(fe, hf, cj); #c1(VEPH1) c2(NP_001161383) c3(ventricular zone-expressed PH domain-containing protein homolog 1 isoform 2) c4(65645) c5(65646, 65647, 65648, 65649, 65650, 65651, 65652, 65653, 65654, 65655) c6( ) c7(T, CT, dY); #c1(VEPH1) c2(NP_001161387) c3(ventricular zone-expressed PH domain-containing protein homolog 1 isoform 3) c4(65656) c5(65657, 65658, 65659, 65660, 65661, 65662, 65663, 65664, 65665, 65666) c6( ) c7(T, CT, dY); #c1(VEPH1) c2(NP_001161389) c3(ventricular zone-expressed PH domain-containing protein homolog 1 isoform 5) c4(65667, 65668) c5(65669, 65670, 65671, 65672, 65673, 65674, 65675, 65676, 65677, 65678, 65679, 65680, 65681, 65682, 65683, 65684, 65685, 65686, 65687, 65688, 65689, 65690, 65691, 65692) c6( ) c7(T, CT, dY); #c1(VEPH1) c2(NP_078897) c3(ventricular zone-expressed PH domain-containing protein homolog 1 isoform 1) c4(215979, 215980, 215981) c5(215982, 215983, 215984, 215985, 215986, 215987, 215988, 215989, 215990, 215991) c6( ) c7(T, CT, dY); #c1(VEZT) c2(NP_060069) c3(vezatin) c4(194027, 194028, 194029) c5(194030, 194031, 194032, 194033, 194034, 194035, 194036, 194037, 194038, 194039, 194040, 194041) c6( ) c7(dP); #c1(VEZT) c2(XP_011536849) c3(vezatin isoform X6) c4( ) c5(334541, 334542, 334543, 334544, 334545, 334546) c6( ) c7(dP); #c1(VGF) c2(XP_011514851) c3(neurosecretory protein VGF isoform X1) c4(307599, 307600) c5(307601, 307602, 307603, 307604, 307605, 307606, 307607, 307608, 307609, 307610, 307611, 307612, 307613, 307614, 307615, 307616) c6( ) c7(bL, b, bS, ci, ct, bxy, dL, bK, K, he, eL, af, dL, yq, Z, t, iK, avf, bN, c, cA, m); #c1(VGLL1) c2(NP_057351) c3(transcription cofactor vestigial-like protein 1) c4(190269, 190270, 190271, 190272) c5(190273, 190274, 190275, 190276, 190277, 190278, 190279, 190280, 190281, 190282, 190283, 190284, 190285, 190286, 190287, 190288, 190289, 190290, 190291, 190292, 190293, 190294, 190295, 190296) c6( ) c7(asT, aW, ci, AO, aT); #c1(VGLL2) c2(NP_703154) c3 (transcription cofactor vestigial-like protein 2 isoform 2) c4(246069) c5(246070, 246071, 246072, 246073, 246074, 246075) c6( ) c7(Y); #c1(VGLL2) c2(NP_872586) c3(transcription cofactor vestigial-like protein 2 isoform 1) c4(256436) c5(256437, 256438, 256439, 256440, 256441, 256442) c6( ) c7(Y); #c1(VGLL3) c2(NP_057290) c3(transcription cofactor vestigial-like protein 3) c4(189911) c5(189912, 189913, 189914, 189915, 189916, 189917) c6( ) c7(bA, t, v, m, ad); #c1(VGLL3) c2(XP_005264809) c3(transcription cofactor vestigial-like protein 3 isoform X2) c4(279248) c5(279249, 279250, 279251, 279252, 279253, 279254) c6( ) c7(bA, t, v, m, ad); #c1(VGLL4) c2(NP_001121691) c3(transcription cofactor vestigial-like protein 4 isoform a) c4(46070) c5(46071, 46072, 46073, 46074, 46075, 46076) c6( ) c7(T, iX, aA, aF, j, bK); #c1(VGLL4) c2(NP_001121692) c3(transcription cofactor vestigial-like protein 4 isoform c) c4(46077) c5(46078, 46079, 46080, 46081, 46082, 46083) c6( ) c7(T, iX, aA, aF, j, bK); #c1(VGLL4) c2(NP_001121693) c3(transcription cofactor vestigial-like protein 4 isoform d) c4(46084) c5(46085, 46086, 46087, 46088, 46089, 46090) c6( ) c7(T, iX, aA, aF, j, bK); #c1(VGLL4) c2(NP_001271319) c3(transcription cofactor vestigial-like protein 4 isoform e) c4(103890) c5(103891, 103892, 103893, 103894, 103895, 103896) c6( ) c7(T, iX, aA, aF, j, bK); #c1(VGLL4) c2(NP_055482) c3(transcription cofactor vestigial-like protein 4 isoform b) c4(181377, 181378, 181379, 181380, 181381) c5(181382, 181383, 181384, 181385, 181386, 181387, 181388, 181389, 181390, 181391, 181392, 181393, 181394, 181395, 181396, 181397, 181398, 181399, 181400, 181401, 181402, 181403, 181404, 181405, 181406, 181407, 181408, 181409, 181410, 181411) c6( ) c7(T, iX, aA, aF, j, bK); #c1(VHL) c2(NP_000542) c3(von Hippel-Lindau disease tumor suppressor isoform 1) c4(12426, 12427) c5(12428, 12429, 12430, 12431, 12432, 12433, 12434, 12435, 12436, 12437, 12438, 12439) c6( ) c7(atT, T, aC, e, ci, aY, Hr, ip, lS, awe, bll, pX, K, Nj, MO, aSl, eL, Us, De, aW, dg, zC, g, as, cm, bw, qJ, eM, nB, iA, go, cj, QF, m, ym, czO, WA, fr, rP, bkU, ft, Do, dL, ni, YI, Hu, bzv, bP, dB, Vy, aoB, anx, nz, cFl, ul, dA, eR, aA, hy, lu, q, er, eA, oT, RI, nv, aT, cy, j, sR, blD, s, iI, en, t, auo, hc, bmR, aJS, VB, hl, jh, aNf, Jm, av, ib, py, Db, fb, cTM, bug, lR, ff, and, EZ, bz, dr, bF, es, Ew, bE, Km, aS, bC, oQ, iu, bEt); #c1(VHL) c2(NP_937799) c3(von Hippel-Lindau disease tumor suppressor isoform 2) c4(259132) c5(259133, 259134, 259135, 259136, 259137, 259138) c6( ) c7(atT, T, aC, e, ci, aY, Hr, ip, lS, awe, bll, pX, K, Nj, MO, aSl, eL, Us, de, aW, dg, zC, g, as, cm, bw, qJ, eM, nB, iA, go, cj, QF, m, ym, czO, WA, fr, rP, bkU, ft, Do, dL, ni, YI, Hu, bzv, bP, dB, Vy, aoB, anx, nz, cFl, ul, dA, eR, aA, hy, lu, q, er, eA, oT, RI, nv, aT, cy, jj, sR, blD, s, iI, en, t, auo, hc, bmR, aJS, VB, hl, jh, aNf, Jm, av, ib, py, Db, fb, cTM, bug, lR, ff, and, EZ, bz, dr, bF, es, Ew, bE, Km, aS, bC, oQ, iu, bEt); #c1(VHLL) c2(NP_001004319) c3(von Hippel-Lindau-like protein) c4(20577) c5(20578, 20579, 20580, 20581, 20582, 20583) c6( ) c7(ck, BI, T, RB, aE, QO, aVl, ds, if, aiM, cU, h, lo, EA, qN, aMk); #c1(VIL1) c2(NP_009058) c3(villin-1) c4(170505, 170506, 170507) c5(170508, 170509, 170510, 170511, 170512, 170513, 170514, 170515, 170516, 170517, 170518, 170519, 170520, 170521, 170522, 170523, 170524, 170525, 170526) c6( ) c7(Fv, ai, ft, A, ff, n, cl, ci, en, apx, y, B, o); #c1(VIM) c2(XP_011517951) c3(vimentin isoform X1) c4(311962, 311963, 311964, 311965, 311966) c5(311967, 311968, 311969, 311970, 311971, 311972, 311973, 311974, 311975, 311976, 311977, 311978, 311979, 311980, 311981, 311982, 311983, 311984, 311985, 311986, 311987, 311988, 311989, 311990, 311991, 311992, 311993, 311994, 311995, 311996, 311997, 311998, 311999, 312000) c6( ) c7(gh, T, fR, pf, hV, ea, al, bB, cL, ci, ip, bv, aE, iv, K, bOZ, eL, li, aW, Qm, g, as, pL, eF, cm, Qx, iP, eM, Jr, nB, iA, U, cj, bAD, Hb, m, zv, Th, dD, cTN, Ol, Or, qy, asM, n, fr, qN, PJ, aOr, ai, EI, ke, b, jz, Tf, eV, dL, Xh, dx, YO, OV, hQ, aq, mo, ceO, eD, va, bk, it, B, arO, cn, pF, fM, jp, DD, KR, bBr, jx, eX, aA, v, q, o, er, eA, PC, XX, hW, BJ, yg, aT, j, ae, jf, cPm, s, nO, iU, Y, fO, r, en, cl, t, iK, rY, ck, pa, iM, kP, Dy, AH, bdP, hr, adR, dy, EY, JH, fD, zu, bt, Mb, kh, eP, aaz, iW, ha, jh, eq, bNK, iJ, ad, wD, hX, abZ, av, hd, ib, d, je, xz, eH, ff, aJK, aF, bz, ek, dR, LB, es, gk, eU, bE, i, y, kS, iu); #c1(VIMP) c2(NP_060915) c3 (selenoprotein S isoform 2) c4(198601) c5( ) c6( ) c7(bL, bM, am, dD, e, aA, aj, v, j, eq, q, Tz, J, ad, xy, M, K, A, r, dq, uC, fT, ac, eM, y, B); #c1(VIMP) c2(NP_982298) c3(selenoprotein S isoform 1) c4(263315) c5(263316, 263317, 263318, 263319, 263320, 263321, 263322, 263323, 263324, 263325, 263326, 263327, 263328, 263329, 263330) c6( ) c7(bL, bM, am, dD, e, aA, aj, v, j, eq, q, Tz, J, ad, xy, M, K, A, r, dq, uC, fT, ac, eM, y, B); #c1(VIP) c2(NP_003372) c3(VIP peptides isoform 1 preproprotein) c4(137846) c5(137847, 137848, 137849, 137850, 137851, 137852, 137853, 137854, 137855, 137856, 137857, 137858, 137859, 137860, 137861, 137862, 137863, 137864, 137865, 137866, 137867, 137868, 137869, 137870, 137871, 137872, 137873, 137874, 137875, 137876, 137877, 137878, 137879, 137880, 137881, 137882, 137883, 137884, 137885, 137886, 137887, 137888, 137889, 137890, 137891, 137892, 137893, 137894, 137895, 137896, 137897, 137898, 137899, 137900, 137901, 137902, 137903, 137904, 137905, 137906, 137907, 137908, 137909, 137910, 137911, 137912, 137913, 137914, 137915, 137916, 137917, 137918, 137919, 137920, 137921, 137922, 137923, 137924, 137925, 137926, 137927, 137928, 137929, 137930, 137931, 137932, 137933, 137934, 137935, 137936, 137937, 137938, 137939, 137940) c6(137941) c7(cDT, gh, T, bM, lD, bB, ci, Jx, ip, xE, rw, iv, Ds, bzw, de, ly, bn, aW, mO, g, eE, cm, eM, U, vy, cZ, BH, akA, nA, qW, ai, cU, h, dd, he, dx, mR, RT, cOy, sp, lx, JM, at, uz, Dw, v, V, pd, aT, cy, bK, ae, eu, jf, hM, fO, f, aTQ, bN, EJ, cT, Ta, ac, c, EY, fD, ao, bNx, gx, jN, uu, ad, BY, Tg, lC, ji, lQ, aNR, hp, bz, qa, bE, rH, ei, y, Tc); #c1(VIP) c2(NP_919416) c3(VIP peptides isoform 2 preproprotein) c4(258553) c5(258554, 258555, 258556, 258557, 258558, 258559, 258560) c6( ) c7(cDT, gh, T, bM, lD, bB, ci, Jx, ip, xE, rw, iv, Ds, bzw, De, ly, bn, aW, mO, g, eE, cm, eM, U, vy, cZ, BH, akA, nA, qW, ai, cU, h, dd, he, dx, mR, RT, cOy, sp, lx, JM, at, uz, Dw, v, V, pd, aT, cy, bK, ae, eu, jf, hM, fO, f, aTQ, bN, EJ, cT, Ta, ac, c, EY, fD, ao, bNx, gx, jN, uu, ad, BY, Tg, lC, ji, lQ, aNR, hp, bz, qa, bE, rH, ei, y, Tc); #c1(VIP) c2(XP_006715625) c3(VIP peptides isoform X1) c4(289680) c5(289681, 289682, 289683, 289684, 289685, 289686, 289687) c6( ) c7(cDT, gh, T, bM, lD, bB, ci, Jx, ip, xE, rw, iv, Ds, bzw, de, ly, bn, aW, m, g, eE, cm, eM, U, vy, cZ, BH, akA, nA, qW, ai, cU, h, dd, he, dx, mR, RT, cOy, sp, lx, JM, at, uz, Dw, v, V, pd, aT, cy, bK, ae, eu, jf, hM, fO, f, aTQ, bN, EJ, cT, Ta, ac, c, EY, fD, ao, bNx, gx, jN, uu, ad, BY, Tg, lC, ji, lQ, aNR, hp, bz, qa, bE, rH, ei, y, Tc); #c1(VIPAS39) c2(NP_001180244) c3(spermatogenesis-defective protein 39 homolog isoform 1) c4(73518, 73519, 73520, 73521) c5(73522, 73523, 73524, 73525, 73526, 73527, 73528, 73529, 73530, 73531, 73532, 73533, 73534, 73535, 73536, 73537, 73538, 73539, 73540, 73541, 73542, 73543, 73544, 73545, 73546, 73547) c6( ) c7(IM, cTO, afk, lk); #c1(VIPAS39) c2(NP_001180245) c3(spermatogenesis-defective protein 39 homolog isoform 2) c4(73548) c5(73549, 73550, 73551, 73552, 73553, 73554, 73555, 73556, 73557, 73558, 73559, 73560) c6( ) c7(IM, cTO, afk, lk); #c1(VIPR1) c2(NP_001238811) c3(vasoactive intestinal polypeptide receptor 1 isoform 2) c4( ) c5(87915, 87916, 87917, 87918, 87919) c6( ) c7(Kd, T, fR, ya, boZ, al, jx, bB, n, uu, apt, q, ad, F, aZv, qh, aT, h, iY, bK, adV, ae, dx, mR, bn, aW, vj, ff, fO, hl, r, kD, aq, lo, fe, EZ, en, g, iV, f, cm, bz, bN, TA, gk, bE, hj, at, qF, hE, B, y, vi); #c1(VIPR1) c2(NP_001238812) c3(vasoactive intestinal polypeptide receptor 1 isoform 3) c4(87920) c5(87921, 87922, 87923, 87924, 87925, 87926) c6( ) c7(Kd, T, fR, ya, boZ, al, jx, bB, n, uu, apt, q, ad, F, aZv, qh, aT, h, iY, bK, adV, ae, dx, mR, bn, aW, vj, ff, fO, hl, r, kD, aq, lo, fe, EZ, en, g, iV, f, cm, bz, bN, TA, gk, bE, hj, at, qF, hE, B, y, vi); #c1(VIPR1) c2(NP_001238813) c3(vasoactive intestinal polypeptide receptor 1 isoform 4) c4(87927) c5(87928, 87929, 87930, 87931, 87932, 87933) c6( ) c7(Kd, T, fR, ya, boZ, al, jx, bB, n, uu, apt, q, ad, F, aZv, qh, aT, h, iY, bK, adV, ae, dx, mR, bn, aW, vj, ff, fO, hl, r, kD, aq, lo, fe, EZ, en, g, iV, f, cm, bz, bN, TA, gk, bE, hj, at, qF, hE, B, y, vi); #c1(VIPR1) c2(NP_001238814) c3(vasoactive intestinal polypeptide receptor 1 isoform 5) c4(87934) c5( ) c6( ) c7(Kd, T, FR, ya, boZ, al, jx, bB, n, uu, apt, q, ad, F, aZv, qh, aT, h, iY, bK, adV, ae, dx, mR, bn, aW, vj, ff, fO, hl, r, kD, aq, lo, fe, EZ, en, g, iV, f, cm, bz, bN, TA, gk, bE, hj, at, qF, hE, B, y, vi); #c1(VIPR1) c2(NP_004615) c3(vasoactive intestinal polypeptide receptor 1 isoform 1 precursor) c4(148187) c5(148188, 148189, 148190, 148191, 148192, 148193, 148194) c6( ) c7(Kd, T, fR, ya, boZ, al, jx, bB, n, uu, apt, q, ad, F, aZv, qh, aT, h, iY, bK, adV, ae, dx, mR, bn, aW, vj, ff, fO, hl, r, kD, aq, lo, fe, EZ, en, g, iV, f, cm, bz, bN, TA, gk, bE, hj, at, qF, hE, B, y, vi); #c1(VIPR2) c2(NP_001291451) c3(vasoactive intestinal polypeptide receptor 2 isoform 2 precursor) c4( ) c5(116074, 116075, 116076, 116077, 116078, 116079) c6( ) c7(ae, T, NC, ac, Ei, Be, cm, uu, eo, ei, eE, Bd, V, bz, ai, dx, ad); #c1(VIPR2) c2(NP_003373) c3(vasoactive intestinal polypeptide receptor 2 isoform 1 precursor) c4(137942, 137943) c5(137944, 137945, 137946, 137947, 137948, 137949) c6( ) c7(ae, T, NC, ac, Ei, Be, cm, uu, eo, ei, eE, Bd, V, bz, ai, dx, ad); #c1(VIT) c2(NP_001171440) c3(vitrin isoform 2 precursor) c4(70087) c5(70088, 70089, 70090, 70091, 70092, 70093, 70094, 70095, 70096) c6(70097, 70098) c7(fq, ac); #c1(VIT) c2(NP_001171441) c3(vitrin isoform 3 precursor) c4(70099) c5(70100, 70101, 70102, 70103, 70104, 70105, 70106, 70107) c6(70108, 70109) c7(fq, ac); #c1(VIT) c2(NP_001171442) c3(vitrin isoform 4 precursor) c4(70110, 70111) c5(70112, 70113, 70114, 70115, 70116, 70117, 70118, 70119) c6(70120, 70121) c7(fq, ac); #c1(VIT) c2(NP_001171443) c3(vitrin isoform 5 precursor) c4(70122) c5(70123, 70124, 70125, 70126, 70127, 70128, 70129, 70130) c6(70131, 70132) c7(fq, ac); #c1(VIT) c2(NP_444506) c3(vitrin isoform 1 precursor) c4(230323) c5(230324, 230325, 230326, 230327, 230328, 230329, 230330, 230331) c6(230332, 230333) c7(fq, ac); #c1(VIT) c2(XP_011531207) c3(vitrin isoform X1) c4(327159) c5(327160, 327161, 327162, 327163, 327164, 327165, 327166, 327167) c6(327168, 327169) c7(fq, ac); #c1(VIT) c2(XP_011531208) c3(vitrin isoform X2) c4( ) c5(327170, 327/71, 327172, 327173, 327174, 327175, 327176, 327/77) c6(327178, 327179) c7(fq, ac); #c1(VKORC1) c2(NP_076869) c3(vitamin K epoxide reductase complex subunit 1 isoform 1 precursor) c4(214186) c5(214187, 214188, 214189, 214190, 214191, 214192) c6( ) c7(bL, dD, Ly, mr, ea, wl, e, aw, ez, vr, Mi, j, eq, cd, ad, hX, fU, ga, vh, M, acb, wt, adx, ae, cTP, af, bA, eu, eZ, aq, aaD, cM, ac, ag, fP, gZ, bcu, qq, dX, aS, bcc, lS, vF, ei, wx, B); #c1(VKORC1) c2(NP_996560) c3(vitamin K epoxide reductase complex subunit 1 isoform 2 precursor) c4(264309) c5(264310, 264311, 264312, 264313, 264314, 264315) c6( ) c7(bL, dD, Ly, mr, ea, wl, e, aw, ez, vr, Mi, j, eq, cd, ad, hX, fU, ga, vh, M, acb, wt, adx, ae, cTP, af, bA, eu, eZ, aq, aaD, cM, ac, ag, fP, gZ, bcu, qq, dX, aS, bcc, lS, vF, ei, wx, B); #c1(VLDLR) c2(NP_001018066) c3(very low-density lipoprotein receptor isoform b precursor) c4(27045) c5(27046, 27047, 27048, 27049, 27050, 27051) c6( ) c7(bL, bM, T, dD, ea, bO, aA, cR, bS, ah, ct, C, ci, v, eq, dY, ec, io, vw, cy, K, bq, dL, he, hB, dF, af, hm, Jl, ZC, fc, FD, lo, EH, aF, Z, bN, cT, W, ky, dC, Fi, bhU, adl, xg); #c1(VLDLR) c2(NP_003374) c3(very low-density lipoprotein receptor isoform a precursor) c4(137950) c5(137951, 137952, 137953, 137954, 137955, 137956) c6( ) c7(bL, bM, T, dD, ea, bO, aA, cR, bS, ah, ct, C, ci, v, eq, dY, ec, io, vw, cy, K, bq, dQ, he, hB, dF, af, hm, Jl, ZC, fc, FD, lo, EH, aF, Z, bN, cT, W, ky, dC, Fi, bhU, adl, xg); #c1(VLDLR) c2(XP_011516331) c3(very low-density lipoprotein receptor isoform X1) c4(309530, 309531) c5(309532, 309533, 309534, 309535, 309536, 309537, 309538, 309539, 309540, 309541, 309542, 309543) c6( ) c7(bL, bM, T, dD, ea, bO, aA, cR, bS, ah, ct, C, ci, v, eq, dY, ec, io, vw, cy, K, bq, da, he, hB, dF, af, hm, Jl, ZC, fc, FD, lo, EH, aF, Z, bN, cT, W, ky, dC, Fi, bhU, adl, xg); #c1(VMA21) c2(NP_001017980) c3(vacuolar ATPase assembly integral membrane protein VMA21) c4(26725) c5(26726, 26727, 26728, 26729, 26730, 26731) c6( ) c7(adl, aiW, cTQ, akd, oj); #c1(VMA21) c2(XP_011529427) c3(vacuolar ATPase assembly integral membrane protein VMA21 isoform X1) c4(324855) c5(324856, 324857, 324858, 324859, 324860, 324861) c6( ) c7(adl, aiW, cTQ, akd, oj); #c1(VMO1) c2(NP_001138411) c3(vitelline membrane outer layer protein 1 homolog isoform 2 precursor) c4(56577) c5(56578, 56579, 56580, 56581, 56582, 56583, 56584) c6(56585, 56586) c7(fr); #c1(VMO1) c2(NP_001138412) c3(vitelline membrane outer layer protein 1 homolog isoform 3 precursor) c4(56587) c5(56588, 56589, 56590, 56591, 56592, 56593, 56594) c6(56595, 56596) c7(fr); #c1(VMO1) c2(NP_001138413) c3(vitelline membrane outer layer protein 1 homolog isoform 4 precursor) c4(56597) c5(56598, 56599, 56600, 56601, 56602, 56603) c6(56604) c7(fr); #c1(VMO1) c2(NP_872372) c3(vitelline membrane outer layer protein 1 homolog isoform 1 precursor) c4(256270) c5(256271, 256272, 256273, 256274, 256275, 256276, 256277, 256278, 256279, 256280, 256281) c6(256282, 256283) c7(fr); #c1(VMP1) c2(NP_(12200) c3(vacuole membrane protein 1) c4(220747, 220748, 220749, 220750, 220751) c5(220752, 220753, 220754, 220755, 220756, 220757, 220758, 220759, 220760, 220761, 220762, 220763, 220764, 220765, 220766, 220767, 220768, 220769, 220770, 220771, 220772, 220773, 220774, 220775, 220776, 220777, 220778, 220779, 220780, 220781) c6( ) c7(es, T, aC, ar, aE, iA, wb, K, ey, B); #c1(VN1R2) c2(NP_776255) c3(vomeronasal type-1 receptor 2) c4(251050) c5(251051, 251052, 251053, 251054, 251055, 251056) c6( ) c7(dc, T); #c1(VN1R4) c2(NP_776256) c3(vomeronasal type-1 receptor 4) c4(251057) c5(251058, 251059, 251060, 251061, 251062, 251063, 251064) c6( ) c7(dc, T); #c1(VNN1) c2(NP_004657) c3(pantetheinase precursor) c4(148574) c5(148575, 148576, 148577, 148578, 148579, 148580) c6( ) c7(ac, e, aAh, awd, dV, r, cz, cr, fe, ji, K, cP, iV, eE, q, bA); #c1(VNN2) c2(NP_001229279) c3(vascular non-inflammatory molecule 2 isoform 3 precursor) c4(84113, 84114, 84115, 84116, 84117) c5(84118, 84119, 84120, 84121, 84122, 84123, 84124, 84125, 84126, 84127, 84128, 84129, 84130, 84131, 84132, 84133, 84134, 84135, 84136, 84137, 84138, 84139, 84140, 84141, 84142, 84143, 84144, 84145, 84146, 84147, 84148, 84149, 84150) c6( ) c7(aAh, awd, dV, al, cr, eE, pd); #c1(VNN2) c2(NP_004656) c3(vascular non-inflammatory molecule 2 isoform 1 precursor) c4(148555, 148556, 148557, 148558) c5(148559, 148560, 148561, 148562, 148563, 148564, 148565, 148566, 148567, 148568, 148569, 148570, 148571, 148572, 148573) c6( ) c7(aAh, awd, dV, aT, cr, eE, pd); #c1(VNN2) c2(XP_006715656) c3(vascular non-inflammatory molecule 2 isoform X1) c4( ) c5(289695, 289696, 289697, 289698, 289699) c6( ) c7(aAh, awd, dV, aT, cr, eE, pd); #c1(VNN2) c2(XP_011534533) c3(vascular non-inflammatory molecule 2 isoform X2) c4( ) c5(331045, 331046, 331047, 331048, 331049, 331050, 331051) c6( ) c7(aAh, awd, dV, aT, cr, eE, pd); #c1(VNN3) c2(NP_001278632) c3(vascular non-inflammatory molecule 3) c4( ) c5(109908, 109909) c6( ) c7(eE, cr); #c1(VOPP1) c2(NP_001271212) c3(vesicular, overexpressed in cancer, prosurvival protein 1 isoform 3) c4(103635) c5(103636, 103637, 103638, 103639, 103640) c6( ) c7(bt, ff, en); #c1(VOPP1) c2(NP_110423) c3(vesicular, overexpressed in cancer, prosurvival protein 1 isoform 1 precursor) c4(220263) c5(220264, 220265, 220266, 220267, 220268) c6( ) c7(bt, ff, en); #c1(VOPP1) c2(XP_011513840) c3(vesicular, overexpressed in cancer, prosurvival protein 1 isoform X2) c4( ) c5(306278, 306279) c6( ) c7(bt, ff, en); #c1(VPRBP) c2(NP_055518) c3(protein VPRBP isoform 1) c4(181666, 181667) c5(181668, 181669, 181670, 181671, 181672, 181673, 181674, 181675, 181676, 181677, 181678, 181679, 181680, 181681, 181682, 181683, 181684, 181685, 181686, 181687, 181688, 181689) c6( ) c7(T, y, dA, eM, asP, aW, bo, yG, aT, h, aj, K, m, EA, t, J); #c1(VPRBP) c2(XP_006713491) c3(protein VPRBP isoform X3) c4(287169) c5( ) c6( ) c7(T, y, dA, eM, asP, aW, bo, yG, aT, h, aj, K, m, EA, t, J); #c1(VPRBP) c2(XP_011532582) c3(protein VPRBP isoform X2) c4(328697) c5(328698, 328699, 328700, 328701, 328702, 328703) c6( ) c7(T, y, dA, eM, asP, aW, bo, yG, aT, h, aj, K, m, EA, t, J); #c1(VPREB1) c2(NP_009059) c3(immunoglobulin iota chain isoform 1 precursor) c4(170527, 170528) c5(170529, 170530, 170531, 170532, 170533, 170534, 170535, 170536, 170537, 170538, 170539, 170540) c6( ) c7(ck, x, T, iT, eZ, pk, hf, hF, y, Wt); #c1(VPREB3) c2(NP_037510) c3(pre-B lymphocyte protein 3 precursor) c4(175940) c5(175941, 175942, 175943, 175944, 175945, 175946) c6( ) c7(pk, fM, jh); #c1(VPS11) c2(XP_01154/201) c3(vacuolar protein sorting-associated protein 11 homolog isoform X1) c4( ) c5(339835) c6( ) c7(bE, if, bB, aei, qN, cl, eW); #c1(VPS13A) c2(NP_001018047) c3(vacuolar protein sorting-associated protein 13A isoform C) c4(26901) c5(26902, 26903, 26904, 26905, 26906, 26907) c6( ) c7(bUd, Pw, T, aR, ZM, U, r, aU, aA, zU, yy, bl, bX, iI, xw, bgl, dr); #c1(VPS13A) c2(NP_001018048) c3(vacuolar protein sorting-associated protein 13A isoform D) c4(26908) c5(26909, 26910, 26911, 26912, 26913, 26914) c6( ) c7(bUd, Pw, T, aR, ZM, U, r, aJI, aA, zU, yy, bl, bX, iI, xw, bgl, dr); #c1(VPS13A) c2(NP_056001) c3(vacuolar protein sorting-associated protein 13A isoform B) c4(185068) c5(185069, 185070, 185071, 185072, 185073, 185074) c6( ) c7(bUd, Pw, T, aR, ZM, U, r, aJI, aA, zU, yy, bl, bX, iI, xw, bgl, dr); #c1(VPS13A) c2(NP_150648) c3(vacuolar protein sorting-associated protein 13A isoform A) c4(228254) c5(228255, 228256, 228257, 228258, 228259, 228260) c6( ) c7(bUd, Pw, T, aR, ZM, U, r, aJI, aA, zU, yy, bl, bX, iI, xw, bgl, dr); #c1(VPS13B) c2(NP_056058) c3 (vacuolar protein sorting-associated protein 13B isoform 3) c4(185432) c5(185433, 185434, 185435, 185436, 185437, 185438) c6( ) c7(gV, rT, hG, hk, Y, mG, XC, QK, af, bA); #c1(VPS13B) c2(NP_689777) c3(vacuolar protein sorting-associated protein 138 isoform 1) c4(243931, 243932) c5(243933, 243934, 243935, 243936, 243937, 243938) c6( ) c7(gV, rT, hG, hk, Y, mG, XC, QK, af, bA); #c1(VPS13B) c2(NP_858047) c3(vacuolar protein sorting-associated protein 13B isoform 4) c4(255312) c5(255313, 255314, 255315, 255316, 255317, 255318) c6( ) c7(gV, rT, hG, hk, Y, mG, XC, QK, af, bA); #c1(VPS13B) c2(XP_005250858) c3(vacuolar protein sorting-associated protein 13B isoform X1) c4(270611) c5(270612, 270613, 270614, 270615, 270616, 270617) c6( ) c7(gV, rT, hG, hk, Y, mG, XC, QK, af, bA); #c1(VPS13B) c2(XP_011515154) c3(vacuolar protein sorting-associated protein 13B isoform X5) c4( ) c5(307996) c6( ) c7(gV, rT, hG, hk, Y, mG, XC, QK, af, bA); #c1(VPS13B) c2(XP_011515157) c3(vacuolar protein sorting-associated protein 13B isoform X9) c4( ) c5(307997, 307998, 307999, 308000, 308001) c6( ) c7(gV, rT, hG, hk, Y, mG, XC, QK, af, bA); #c1(VPS13C) c2(NP_001018098) c3(vacuolar protein sorting-associated protein 13C isoform 2B) c4(27326) c5( ) c6( ) c7(bL, aj, v, J); #c1(VPS13C) c2(NP_060154) c3(vacuolar protein sorting-associated protein 13C isoform (A) c4(194512) c5(194513, 194514, 194515, 194516, 194517, 194518, 194519, 194520, 194521, 194522, 194523, 194524) c6( ) c7(bL, aj, v, J); #c1(VPS13C) c2(NP_060550) c3(vacuolar protein sorting-associated protein 13C isoform (B) c4(196529) c5( ) c6( ) c7(bL, aj, v, J); #c1(VPS13C) c2(NP_065872) c3(vacuolar protein sorting-associated protein 13C isoform 2A) c4(205989) c5(205990, 205991, 205992, 205993, 205994, 205995, 205996, 205997, 205998, 205999, 206000, 206001) c6( ) c7(bL, aj, v, J); #c1(VPS26A) c2(NP_001030337) c3(vacuolar protein sorting-associated protein 26A isoform 2) c4(31020) c5(31021, 31022, 31023, 31024, 31025, 31026) c6( ) c7(bL, dL); #c1(VPS26A) c2(NP_004887) c3(vacuolar protein sorting-associated protein 26A isoform 1) c4(150545, 150546) c5(150547, 150548, 150549, 150550, 150551, 150552, 150553, 150554, 150555, 150556, 150557, 150558, 150559, 150560, 150561, 150562, 150563) c6( ) c7(bL, dL); #c1(VPS26A) c2(XP_011538680) c3(vacuolar protein sorting-associated protein 26A isoform X1) c4(336417) c5(336418, 336419, 336420, 336421, 336422) c6( ) c7(bL, dL); #c1(VPS28) c2(NP_057292) c3(vacuolar protein sorting-associated protein 28 homolog isoform 1) c4(189934) c5(189935, 189936, 189937, 189938, 189939, 189940, 189941, 189942, 189943, 189944) c6( ) c7(h, ds); #c1(VPS28) c2(NP_898880) c3(vacuolar protein sorting-associated protein 28 homolog isoform 2) c4(257807) c5(257808, 257809, 257810, 257811, 257812, 257813) c6( ) c7(h, ds); #c1(VPS33A) c2(NP_075067) c3(vacuolar protein sorting-associated protein 33A) c4(213655) c5(213656, 213657, 213658, 213659, 213660, 213661) c6( ) c7(afk, lF); #c1(VPS33B) c2(NP_061138) c3(vacuolar protein sorting-associated protein 33B isoform 1) c4(199491, 199492) c5(199493, 199494, 199495, 199496, 199497, 199498, 199499, 199500, 199501, 199502, 199503, 199504) c6( ) c7(mG, afk, Z, lM, lk, jw); #c1(VPS35) c2(NP_060676) c3(vacuolar protein sorting-associated protein 35) c4(197099, 197100) c5(197101, 197102, 197103, 197104, 197105, 197106, 197107, 197108, 197109, 197110, 197111, 197112) c6( ) c7(bL, T, af, cTR, r, aA, v, an, NN, GB); #c1(VPS36) c2(NP_057159) c3(vacuolar protein-sorting-associated protein 36 isoform 1) c4(189201) c5(189202, 189203, 189204, 189205, 189206, 189207) c6( ) c7(cgf); #c1(VPS37A) c2(NP_001138624) c3(vacuolar protein sorting-associated protein 37A isoform 2) c4(57254) c5(57255, 57256, 57257, 57258, 57259, 57260) c6( ) c7(cTS, TP, oe, EQ, m, t); #c1(VPS37A) c2(NP_689628) c3(vacuolar protein sorting-associated protein 37A isoform 1) c4(243339) c5(243340, 243341, 243342, 243343, 243344, 243345) c6( ) c7(cTS, TP, oe, EQ, m, t); #c1(VPS37A) c2(XP_006716349) c3(vacuolar protein sorting-associated protein 37A isoform X1) c4(290262) c5(290263, 290264, 290265, 290266, 290267, 290268) c6( ) c7(cTS, TP, De, EQ, m, t); #c1(VPS37B) c2(NP_078943) c3(vacuolar protein sorting-associated protein 37B) c4(216209) c5(216210, 216211, 216212, 216213, 216214, 216215) c6( ) c7(h); #c1(VPS37B) c2(XP_011537044) c3(vacuolar protein sorting-associated protein 378 isoform X1) c4( ) c5(334830) c6( ) c7(h); #c1(VPS37C) c2(NP_060436) c3(vacuolar protein sorting-associated protein 37C) c4(195925, 195926, 195927) c5(195928, 195929, 195930, 195931, 195932, 195933, 195934, 195935, 195936, 195937, 195938, 195939, 195940, 195941, 195942, 195943, 195944, 195945, 195946) c6( ) c7(ck, h); #c1(VPS39) c2(NP_001288067) c3(vam6/Vps39-like protein isoform a) c4(113484) c5(113485, 113486, 113487, 113488, 113489, 113490) c6( ) c7(aT); #c1(VPS39) c2(NP_056104) c3(vam6/Vps39-like protein isoform b) c4(185726) c5(185727, 185728, 185729, 185730, 185731, 185732) c6( ) c7(aT); #c1(VPS39) c2(XP_011519705) c3(vam6/Vps39-like protein isoform X1) c4( ) c5(314377) c6( ) c7(al); #c1(VPS41) c2(NP_055211) c3(vacuolar protein sorting-associated protein 41 homolog isoform 1) c4(179854, 179855) c5(179856, 179857, 179858, 179859, 179860, 179861, 179862, 179863, 179864, 179865, 179866, 179867) c6( ) c7(T, dY, an); #c1(VPS41) c2(NP_542198) c3(vacuolar protein sorting-associated protein 41 homolog isoform 2) c4(232273) c5(232274, 232275, 232276, 232277, 232278, 232279) c6( ) c7(T, dY, an); #c1(VPS4A) c2(NP_037377) c3(vacuolar protein sorting-associated protein 4A) c4(175145) c5(175146, 175147, 175148, 175149, 175150, 175151) c6( ) c7(afu); #c1(VPS4B) c2(NP_004860) c3(vacuolar protein sorting-associated protein 4B) c4(150378) c5(150379, 150380, 150381, 150382, 150383, 150384) c6( ) c7(aW, er, aT); #c1(VPS51) c2(NP_037397) c3(vacuolar protein sorting-associated protein 51 homolog) c4(175259) c5(175260, 175261, 175262, 175263, 175264, 175265) c6( ) c7(ID, T, aC, ea, e, ci, xE, lv, pX, M, dV, K, eL, tw, ly, eG, aW, Qm, as, bw, eM, AM, vA, mx, vy, Hb, m, fy, dD, vl, aw, n, aj, ww, Mi, fr, eh, cd, br, EI, ai, do, wG, gd, h, dL, ey, cY, A, Vt, kp, mo, op, lx, hq, bm, B, aA, CR, tc, cz, v, q, J, eA, eJ, rS, aT, ae, r, cl, t, ck, kP, wl, dh, dy, bt, kh, dG, eq, ad, Jm, sN, d, lR, tb, aF, qa, Hp, lI, ei, lf, y); #c1(VPS52) c2(NP_001276105) c3(vacuolar protein sorting-associated protein 52 homolog isoform 4) c4( ) c5(108002, 108003, 108004, 108005, 108006, 108007, 108008, 108009, 108010) c6( ) c7(T, Ht, dA, pf, ha, bO, bB, GC, gA, ww, zU, XM, CTT, ad, iu, ku, cQZ, QU, dV, aT, vH, K, fb, ae, li, tb, iI, aW, Y, eZ, agj, pL, wA, bz, hq, nM, bE, PI, yP, hr, uz, abd, B); #c1(VPS52) c2(NP_072047) c3(vacuolar protein sorting-associated protein 52 homolog isoform 1) c4(212497, 212498, 212499, 212500) c5(212501, 212502, 212503, 212504, 212505, 212506, 212507, 212508, 212509, 212510, 212511, 212512, 212513, 212514, 212515, 212516, 212517, 212518, 212519, 212520, 212521, 212522, 212523, 212524, 212525, 212526, 212527, 212528, 212529, 212530, 212531, 212532, 212533, 212534, 212535, 212536, 212537, 212538, 212539, 212540, 212541, 212542, 212543, 212544, 212545, 212546, 212547, 212548, 212549, 212550, 212551, 212552, 212553, 212554, 212555, 212556, 212557, 212558, 212559, 212560, 212561, 212562, 212563, 212564, 212565, 212566, 212567) c6( ) c7(T, Ht, dA, pf, ha, bO, bB, GC, gA, ww, zU, XM, cTT, ad, iu, ku, cQZ, QU, dV, aT, vH, K, fb, ae, li, tb, iU, aW, Y, eZ, agj, pL, wA, bz, hq, nM, bE, PI, yP, hr, uz, abd, B); #c1(VPS53) c2(NP_001121631) c3(vacuolar protein sorting-associated protein 53 homolog isoform 1) c4(45892, 45893, 45894) c5(45895, 45896, 45897, 45898, 45899, 45900, 45901, 45902, 45903, 45904, 45905, 45906, 45907, 45908, 45909, 45910, 45911, 45912) c6( ) c7(T, if, r, aq, q, qN, iR, B); #c1(VPS53) c2(NP_060759) c3(vacuolar protein sorting-associated protein 53 homolog isoform 2) c4(197649, 197650) c5(197651, 197652, 197653, 197654, 197655, 197656) c6( ) c7(T, if, r, aq, q, qN, iR, B); #c1(VPS53) c2(XP_011522255) c3(vacuolar protein sorting-associated protein 53 homolog isoform X1) c4(316896) c5(316897, 316898, 316899, 316900, 316901, 316902, 316903, 316904, 316905, 316906, 316907, 316908) c6( ) c7(T, if, r, aq, q, qN, iR, B); #c1(VPS54) c2(NP_001005739) c3(vacuolar protein sorting-associated protein 54 isoform 2) c4(22000) c5(22001, 22002, 22003, 22004, 22005, 22006, 22007, 22008, 22009, 22010) c6( ) c7(apl, c, apq, HC, PJ); #c1(VPS54) c2(NP_057600) c3(vacuolar protein sorting-associated protein 54 isoform 1) c4(191558, 191559) c5(191560, 191561, 191562, 191563, 191564, 191565, 191566, 191567, 191568, 191569, 191570, 191571) c6( ) c7(apl, c, apq, HC, PJ); #c1(VPS72) c2(NP_001258016) c3(vacuolar protein sorting-associated protein 72 homolog isoform 1) c4(95905) c5(95906, 95907, 95908, 95909, 95910, 95911) c6( ) c7(bz, eM); #c1(VPS72) c2(NP_005988) c3(vacuolar protein sorting-associated protein 72 homolog isoform 2) c4(161010) c5(161011, 161012, 161013, 161014, 161015, 161016, 161017) c6( ) c7(bz, eM); #c1(VPS8) c2(NP_001009921) c3(vacuolar protein sorting-associated protein 8 homolog isoform a) c4(23713, 23714, 23715) c5(23716, 23717, 23718, 23719, 23720, 23721, 23722, 23723, 23724, 23725, 23726, 23727) c6( ) c7(an); #c1(VPS8) c2(NP_056118) c3(vacuolar protein sorting-associated protein 8 homolog isoform b) c4(185770, 185771, 185772, 185773, 185774) c5(185775, 185776, 185777, 185778, 185779, 185780, 185781, 185782, 185783, 185784, 185785, 185786) c6( ) c7(an); #c1(VPS8) c2(XP_005247310) c3(vacuolar protein sorting-associated protein 8 homolog isoform X2) c4( ) c5(267766, 267767, 267768, 267769, 267770, 267771, 267772, 267773, 267774, 267775, 267776, 267777, 267778, 267779, 267780, 267781, 267782, 267783, 267784, 267785, 267786, 267787, 267788, 267789) c6( ) c7(an); #c1(VRK1) c2(NP_003375) c3(serine/threonine-protein kinase VRK1) c4(137957) c5(137958, 137959, 137960, 137961, 137962, 137963) c6( ) c7(av, T, bP, bol, aW, ff, jx, aT, kf, fe, aj, UR, ci, Ea, nA, en, g, bz, eW); #c1(VRK2) c2(NP_001123953) c3(serine/threonine-protein kinase VRK2 isoform 1) c4(48119, 48120, 48121) c5(48122, 48123, 48124, 48125, 48126, 48127, 48128, 48129, 48130, 48131, 48132, 48133, 48134, 48135, 48136, 48137) c6( ) c7(T, Cq, aW, ha, aT, bK, ci); #c1(VRK2) c2(NP_001123954) c3(serine/threonine-protein kinase VRK2 isoform 2) c4(48138) c5( ) c6( ) c7(T, Cq, aW, ha, aT, bk, ci); #c1(VRK2) c2(XP_006712154) c3(serine/threonine-protein kinase VRK2 isoform X2) c4(286461) c5(286462, 286463, 286464, 286465, 286466, 286467, 286468, 286469, 286470, 286471, 286472, 286473, 286474, 286475, 286476, 286477, 286478, 286479) c6( ) c7(T, Cq, aW, ha, aT, bK, ci); #c1(VSIG1) c2(NP_001164024) c3(V-set and immunoglobulin domain-containing protein 1 isoform 1 precursor) c4(66517) c5(66518, 66519, 66520, 66521, 66522, 66523) c6( ) c7(t, aF, aA); #c1(VSIG1) c2(NP_872413) c3(V-set and immunoglobulin domain-containing protein 1 isoform 2 precursor) c4(256317) c5(256318, 256319, 256320, 256321, 256322, 256323) c6( ) c7(t, aF, aA); #c1(VSIG10) c2(NP_061959) c3(V-set and immunoglobulin domain-containing protein 10 precursor) c4(201311) c5(201312, 201313, 201314, 201315, 201316, 201317) c6( ) c7(bL); #c1(VSIG2) c2(NP_055127) c3(V-set and immunoglobulin domain-containing protein 2 precursor) c4(179112, 179113) c5(179114, 179115, 179116, 179117, 179118, 179119, 179120, 179121, 179122, 179123) c6( ) c7(cTU, eZ, hO, dL, K, eL); #c1(VSIG2) c2(XP_011541029) c3(V-set and immunoglobulin domain-containing protein 2 isoform X1) c4( ) c5(339561, 339562) c6( ) c7(cTU, eZ, hO, dL, K, eL); #c1(VSIG2) c2(XP_011541030) c3(V-set and immunoglobulin domain-containing protein 2 isoform X2) c4( ) c5(339563) c6( ) c7(cTU, eZ, hO, dL, K, eL); #c1(VSIG4) c2(NP_001093901) c3(V-set and immunoglobulin domain-containing protein 4 isoform 2 precursor) c4(40552) c5(40553, 40554, 40555, 40556, 40557, 40558) c6( ) c7(eM, ey, ci, bt, bz, ai); #c1(VSIG4) c2(NP_001171759) c3(V-set and immunoglobulin domain-containing protein 4 isoform 4 precursor) c4( ) c5(71514, 71515, 71516, 71517, 71518, 71519, 71520, 71521, 71522, 71523, 71524, 71525) c6( ) c7(BM, ey, ci, bt, bz, ai); #c1(VSIG4) c2(NP_001244332) c3(V-set and immunoglobulin domain-containing protein 4 isoform 5 precursor) c4(92189) c5(92190, 92191, 92192, 92193, 92194, 92195) c6( ) c7(eM, ey, ci, bt, bz, ai); #c1(VSIG4) c2(NP_009199) c3(V-set and immunoglobulin domain-containing protein 4 isoform 1 precursor) c4(171475, 171476, 171477) c5(171478, 171479, 171480, 171481, 171482, 171483) c6( ) c7(eM, ey, ci, bt, bz, ai); #c1(VSNL1) c2(NP_003376) c3(visinin-like protein 1) c4(137964, 137965, 137966) c5(137967, 137968, 137969, 137970, 137971, 137972, 137973, 137974, 137975, 137976, 137977, 137978, 137979, 137980, 137981, 137982, 137983, 137984, 137985, 137986, 137987, 137988, 137989, 137990, 137991, 137992) c6( ) c7(cZ, T, c, af, eM, ff, eH, r, cm, K, bz, en, eA, H, adg, xg, q, L); #c1(VSTM1) c2(NP_001275720) c3(V-set and transmembrane domain-containing protein 1 isoform 2) c4(107/77) c5(107/78, 107179, 107180, 107181, 107182, 107183) c6( ) c7(cd); #c1(VSTM1) c2(NP_001275721) c3(V-set and transmembrane domain-containing protein 1 isoform 3 precursor) c4(107184) c5(107185, 107186, 107187, 107188, 107189, 107190, 107191, 107192, 107193, 107194, 107195, 107196) c6( ) c7(cd); #c1(VSTM1) c2(NP_001275722) c3(V-set and transmembrane domain-containing protein 1 isoform 4) c4( ) c5(107197) c6( ) c7(cd); #c1(VSTM1) c2(NP_940883) c3(V-set and transmembrane domain-containing protein 1 isoform 1 precursor) c4(260034) c5(260035, 260036, 260037, 260038, 260039, 260040) c6( ) c7(cd); #c1(VSTM1) c2(XP_011545826) c3(V-set and transmembrane domain-containing protein 1 isoform X5) c4(344760) c5( ) c6( ) c7(cd); #c1(VSX1) c2(NP_001243200) c3(visual system homeobox 1 isoform c) c4(90136) c5(90137, 90138, 90139, 90140, 90141, 90142, 90143, 90144, 90145, 90146, 90147, 90148, 90149, 90150, 90151, 90152, 90153, 90154) c6( ) c7(gh, T, aSP, e, cTV, zU, Ll, ad, cTW, LH, aOu, aE, ec, ab, vw, LG, ae, Lu, bnk, lR, Az, LJ, W, nb, cA, ig, zG); #c1(VSX1) c2(NP_001243201) c3(visual system homeobox 1 isoform d) c4(90155) c5(90156, 90157, 90158, 90159, 90160, 90161, 90162, 90163, 90164, 90165, 90166, 90167) c6( ) c7(gh, T, aSP, e, cTV, zU, Ll, ad, cTW, LH, aOu, aE, ec, ab, vw, LG, ae, Lu, bnk, lR, Az, LJ, W, nb, cA, ig, zG); #c1(VSX1) c2(NP_055403) c3(visual system homeobox 1 isoform a) c4(180851, 180852, 180853, 180854) c5(180855, 180856, 180857, 180858, 180859, 180860) c6( ) c7(gh, T, aSP, e, cTV, zU, Ll, ad, cTW, LH, aOu, aE, ec, ab, vw, LG, ae, Lu, bnk, lR, Az, LJ, W, nb, cA, ig, zG); #c1(VSX1) c2(NP_955457) c3(visual system homeobox 1 isoform b) c4(261771) c5(261772, 261773, 261774, 261775, 261776, 261777) c6( ) c7(gh, T, aSP, e, cTV, zU, Ll, ad, cTW, LH, aOu, aE, ec, ab, vw, LG, ae, Lu, bnk, lR, Az, LJ, W, nb, cA, ig, zG); #c1(VSX2) c2(NP_878314) c3(visual system homeobox 2) c4(257092) c5(257093, 257094, 257095, 257096, 257097, 257098) c6( ) c7(kn, kj, cTZ, nx, r, cTY, sa, cTX); #c1(VSX2) c2(XP_011535021) c3(visual system homeobox 2 isoform X1) c4( ) c5(331718) c6( ) c7(kn, kj, cTZ, nx, r, cTY, sa, cTX); #c1(VTA1) c2(NP_001273300) c3(vacuolar protein sorting-associated protein VTA1 homolog isoform b) c4( ) c5(104637, 104638, 104639, 104640, 104641) c6( ) c7(T, pf, e, aA, bB, n, q, ad, al, axc, h, dL, K, ae, s, aW, PX, r, aF, g, bz, bE, it, B); #c1(VTA1) c2(NP_001273301) c3(vacuolar protein sorting-associated protein VTA1 homolog isoform c) c4(104642) c5(104643, 104644, 104645, 104646, 104647, 104648) c6( ) c7(T, pf, e, aA, bB, n, q, ad, aT, axc, h, dL, K, ae, s, aW, PX, r, aF, g, bz, bE, it, B); #c1(VTA1) c2(NP_057569) c3(vacuolar protein sorting-associated protein VTA1 homolog isoform a) c4(191380, 191381, 191382) c5(191383, 191384, 191385, 191386, 191387, 191388, 191389, 191390, 191391, 191392, 191393, 191394, 191395) c6( ) c7(T, pf, e, aA, bB, n, q, ad, aT, axc, h, dL, K, ae, s, aW, PX, r, aF, g, bz, bE, it, B); #c1(VTCN1) c2(XP_011540445) c3(V-set domain-containing T-cell activation inhibitor 1 isoform X1) c4(338922) c50) c6( ) c7(T, dA, oe, ht, aA, cl, aj, fr, AD, q, J, o, aE, oz, aT, gs, ey, hu, qw, A, aW, Y, r, kp, hp, aF, t, bz, dy, m, hE, in, y, BA); #c1(VTCN1) c2(XP_011540447) c3(V-set domain-containing T-cell activation inhibitor 1 isoform X3) c4(338923) c5( ) c6( ) c7(T, dA, oe, ht, aA, cl, aj, fr, AD, q, J, o, aE, oz, aT, gs, ey, hu, qw, A, aW, Y, r, kp, hp, aF, t, bz, dy, m, hE, in, y, BA); #c1(VTI1A) c2(NP_660207) c3(vesicle transport through interaction with t-SNAREs homolog (A) c4(240545) c5(240546, 240547, 240548, 240549, 240550, 240551) c6( ) c7(ac, e, aw, r, j, q, g, eu, af, ad); #c1(VTI1A) c2(XP_005269601) c3(vesicle transport through interaction with t-SNAREs homolog 1A isoform X7) c4(282680) c5(282681, 282682, 282683, 282684, 282685, 282686) c6( ) c7(ac, e, aw, r, j, q, g, eu, af, ad); #c1(VTI1B) c2(NP_006361) c3(vesicle transport through interaction with t-SNAREs homolog 1B) c4(164344) c5(164345, 164346, 164347, 164348, 164349, 164350) c6( ) c7(af); #c1(VTN) c2(NP_000629) c3(vitronectin precursor) c4(14090, 14091) c5(14092, 14093, 14094, 14095, 14096, 14097, 14098, 14099, 14100, 14101, 14102, 14103, 14104) c6( ) c7(bL, T, dD, dA, ea, DV, qy, aA, cz, n, j, ci, eq, fr, hF, q, ad, eA, hX, dP, hd, RI, aE, ciJ, tT, dk, aT, cy, db, ae, ajU, hm, afu, d, arl, aW, Y, wc, aaP, aq, lo, YG, aF, CO, g, as, dw, t, bwe, ir, cm, auC, iK, ck, bF, arY, W, cA, pc, eM, DW, aS, m, rH, ei, hh, hE, B, y); #c1(VWA2) c2(NP_001258975) c3(von Willebrand factor A domain-containing protein 2 precursor) c4(96867, 96868) c5(96869, 96870, 96871, 96872, 96873, 96874, 96875, 96876, 96877, 96878, 96879, 96880, 96881, 96882) c6(96883, 96884) c7(aj, aw, X, J, A); #c1(VWA3A) c2(NP_775886) c3(von Willebrand factor A domain-containing protein 3A precursor) c4(250561, 250562) c5(250563, 250564, 250565, 250566, 250567, 250568, 250569, 250570, 250571, 250572, 250573, 250574, 250575, 250576, 250577, 250578) c6(250579, 250580, 250581, 250582) c7(bK); #c1(VWA3A) c2(XP_011544044) c3(von Willebrand factor A domain-containing protein 3A isoform X1) c4(343220) c5(343221) c6( ) c7(bK); #c1(VWA3B) c2(NP_659429) c3(von Willebrand factor A domain-containing protein 3B) c4(239828, 239829) c5(239830, 239831, 239832, 239833, 239834, 239835, 239836, 239837, 239838, 239839, 239840, 239841) c6( ) c7(cd); #c1(VWA3B) c2(XP_006712420) c3(von Willebrand factor A domain-containing protein 3B isoform X6) c4(286641) c5(286642, 286643, 286644, 286645, 286646, 286647) c6( ) c7(cd); #c1(VWA3B) c2(XP_011509074) c3(von Willebrand factor A domain-containing protein 38 isoform X4) c4(299671) c5( ) c6( ) c7(cd); #c1(VWA3B) c2(XP_011509078) c3(von Willebrand factor A domain-containing protein 38 isoform X11) c4( ) c5(299672, 299673, 299674, 299675, 299676, 299677) c6( ) c7(cd); #c1(VWA5A) c2(NP_055437) c3(von Willebrand factor A domain-containing protein 5A isoform 1) c4(181132) c5( ) c6( ) c7(T, aW, Y, aT, ci, qN, an, cp); #c1(VWA5A) c2(NP_938057) c3(von Willebrand factor A domain-containing protein 5A isoform 2) c4( ) c5(259592, 259593, 259594, 259595, 259596, 259597, 259598, 259599, 259600, 259601, 259602, 259603, 259604, 259605, 259606, 259607) c6( ) c7(T, aW, Y, aT, ci, qN, an, cp); #c1(VWA5A) c2(XP_011541130) c3(von Willebrand factor A domain-containing protein 5A isoform X1) c4(339643, 339644) c5(339645, 339646, 339647, 339648, 339649, 339650, 339651, 339652, 339653, 339654) c6( ) c7(T, aW, Y, aT, ci, qN, an, cp); #c1(VWA5B1) c2(XP_011538989) c3(von Willebrand factor A domain-containing protein 5B1 isoform X1) c4(337032) c5(337033, 337034, 337035, 337036, 337037, 337038, 337039, 337040, 337041, 337042, 337043, 337044, 337045, 337046) c6(337047, 337048, 337049, 337050) c7(oe); #c1(VWA5B1) c2(XP_011538990) c3(von Willebrand factor A domain-containing protein 581 isoform X2) c4(337051) c5(337052, 337053, 337054, 337055, 337056, 337057, 337058, 337059) c6(337060, 337061) c7(oe); #c1(VWA5B1) c2(XP_011538996) c3(von Willebrand factor A domain-containing protein 5B1 isoform X7) c4(337062) c5(337063, 337064, 337065, 337066, 337067, 337068, 337069, 337070) c6(337071, 337072) c7(oe); #c1(VWA5B1) c2(XP_011539000) c3(von Willebrand factor A domain-containing protein 5B1 isoform X12) c4(337073) c5(337074, 337075, 337076, 337077, 337078, 337079, 337080, 337081) c6(337082, 337083) c7(oe); #c1(VWA7) c2(NP_079534) c3(von Willebrand factor A domain-containing protein 7 precursor) c4(218705, 218706, 218707) c5(218708, 218709, 218710, 218711, 218712, 218713, 218714, 218715, 218716, 218717, 218718, 218719, 218720, 218721, 218722, 218723, 218724, 218725, 218726, 218727, 218728, 218729, 218730, 218731, 218732, 218733, 218734, 218735, 218736, 218737, 218738, 218739, 218740, 218741, 218742, 218743, 218744, 218745, 218746, 218747, 218748, 218749, 218750, 218751, 218752, 218753, 218754, 218755, 218756, 218757, 218758, 218759, 218760, 218761, 218762, 218763, 218764, 218765, 218766, 218767, 218768, 218769, 218770, 218771, 218772, 218773) c6(218774, 218775, 218776, 218777, 218778, 218779, 218780, 218781, 218782, 218783, 218784, 218785, 218786, 218787, 218788, 218789, 218790, 218791, 218792, 218793) c7(V, A, af, eZ); #c1(VWA7) c2(XP_005249484) c3(von Willebrand factor A domain-containing protein 7 isoform X2) c4( ) c5(269498, 269499, 269500, 269501, 269502, 269503, 269504, 269505, 269506, 269507, 269508, 269509, 269510, 269511, 269512, 269513, 269514, 269515, 269516, 269517, 269518, 269519, 269520, 269521, 269522, 269523, 269524, 269525, 269526, 269527, 269528, 269529, 269530, 269531) c6(269532, 269533, 269534, 269535, 269536, 269537, 269538, 269539, 269540, 269541) c7(V, A, af, eZ); #c1(VWA7) c2(XP_005272961) c3(von Willebrand factor A domain-containing protein 7 isoform X2) c4(284282, 284283) c5( ) c6( ) c7(V, A, af, eZ); #c1(VWA8) c2(NP_001009814) c3(von Willebrand factor A domain-containing protein 8 isoform b precursor) c4(23701, 23702) c5(23703, 23704, 23705, 23706, 23707, 23708, 23709, 23710) c6(23711, 23712) c7(mU, mW, mZ, mS, mY, bK, uu, mV, fq, mT, bN, mX); #c1(VWA8) c2(NP_055873) c3(von Willebrand factor A domain-containing protein 8 isoform a precursor) c4(184071) c5(184072, 184073, 184074, 184075, 184076, 184077, 184078, 184079, 184080, 184081, 184082, 184083, 184084, 184085, 184086, 184087) c6(184088, 184089, 184090, 184091) c7(mU, mW, mZ, mS, mY, bK, uu, mV, fq, mT, bN, mX); #c1(VWCE) c2(NP_689931) c3(von Willebrand factor C and EGF domain-containing protein precursor) c4(244371) c5(244372, 244373, 244374, 244375, 244376, 244377, 244378, 244379) c6(244380, 244381) c7(hX, T, aF, B, aA); #c1(VWDE) c2(NP_001129396) c3(von Willebrand factor D and EGF domain-containing protein precursor) c4(51426, 51427) c5(51428, 51429, 51430, 51431, 51432, 51433, 51434, 51435, 51436, 51437, 51438, 51439, 51440, 51441, 51442) c6(51443, 51444) c7(ad); #c1(VWDE) c2(XP_005249698) c3(von Willebrand factor D and EGF domain-containing protein isoform X3) c4( ) c5(269792, 269793, 269794, 269795, 269796) c6( ) c7(ad); #c1(VWDE) c2(XP_011513477) c3(von Willebrand factor D and EGF domain-containing protein isoform X2) c4(305765) c5( ) c6( ) c7(ad); #c1(VWF) c2(NP_000543) c3(von Willebrand factor preproprotein) c4(12440, 12441, 12442) c5(12443, 12444, 12445, 12446, 12447, 12448, 12449, 12450, 12451, 12452, 12453, 12454, 12455, 12456, 12457, 12458, 12459, 12460, 12461, 12462, 12463, 12464, 12465, 12466, 12467, 12468, 12469, 12470, 12471, 12472, 12473, 12474, 12475, 12476, 12477, 12478, 12479, 12480, 12481, 12482, 12483, 12484, 12485, 12486, 12487, 12488, 12489, 12490, 12491, 12492, 12493, 12494, 12495, 12496, 12497, 12498, 12499, 12500, 12501, 12502, 12503, 12504, 12505, 12506, 12507, 12508, 12509, 12510, 12511, 12512, 12513, 12514, 12515, 12516, 12517, 12518, 12519, 12520, 12521, 12522, 12523, 12524, 12525, 12526, 12527, 12528, 12529, 12530, 12531, 12532, 12533, 12534, 12535, 12536, 12537, 12538, 12539, 12540, 12541, 12542, 12543, 12544, 12545, 12546, 12547, 12548, 12549, 12550, 12551, 12552, 12553, 12554, 12555, 12556, 12557, 12558, 12559, 12560, 12561, 12562, 12563, 12564, 12565, 12566, 12567, 12568, 12569, 12570, 12571, 12572, 12573, 12574, 12575, 12576, 12577, 12578, 12579, 12580, 12581, 12582, 12583, 12584, 12585, 12586, 12587, 12588) c6( ) c7(DS, UH, ahf, vf, ea, e, al, fo, ci, aY, tg, M, dV, vg, adB, K, DY, H, hH, hm, bqB, Bw, hl, vs, eF, uC, DL, y, xK, vy, pJ, P, dD, xu, pA, ZF, cUb, aw, oY, cQ, aj, cUf, cd, eh, ql, Cf, h, yZ, ey, aUH, vx, bpq, wC, A, sq, fe, aUP, aZf, op, ZL, se, Vy, vb, hR, qq, at, cUc, B, fL, L, bL, iH, bpt, aJI, mr, eR, cUe, wL, cz, cR, arw, v, j, C, bX, nS, J, DJ, Wn, DK, ry, cuq, dn, aT, cy, bK, bq, yj, qk, af, eu, cUg, Y, el, cUa, bCY, ac, aUJ, DW, AH, hr, fP, dy, jQ, bt, vi, sY, bgQ, CI, vr, uu, eq, bED, av, vJ, bpp, qh, qV, oW, Os, vc, aUE, vl, azS, UT, cH, vj, eH, kD, aUD, aot, bqz, bz, DI, eK, D, gk, DT, ag, lF, gZ, MJ, dX, aS, DQ, ei, lC, cUd, hv); #c1(WAPAL) c2(NP_055860) c3(wings apart-like protein homolog) c4(183954, 183955) c5(183956, 183957, 183958, 183959, 183960, 183961, 183962, 183963, 183964, 183965, 183966, 183967, 183968, 183969, 183970, 183971) c6( ) c7(T); #c1(WAPAL) c2(XP_011537849) c3(wings apart-like protein homolog isoform X1) c4( ) c5(335364, 335365, 335366, 335367, 335368, 335369, 335370, 335371, 335372, 335373) c6( ) c7(T); #c1(WARS) c2(XP_006720312) c3(tryptophan-tRNA ligase, cytoplasmic isoform X1) c4(293352, 293353, 293354, 293355, 293356, 293357, 293358, 293359, 293360, 293361, 293362, 293363, 293364, 293365, 293366, 293367, 293368, 293369, 293370, 293371, 293372, 293373, 293374) c5(293375, 293376, 293377, 293378, 293379, 293380, 293381, 293382, 293383, 293384, 293385, 293386, 293387, 293388, 293389, 293390, 293391, 293392, 293393, 293394, 293395, 293396, 293397, 293398, 293399, 293400, 293401, 293402, 293403, 293404, 293405, 293406, 293407, 293408, 293409, 293410, 293411, 293412, 293413, 293414, 293415, 293416, 293417, 293418, 293419, 293420, 293421, 293422, 293423, 293424, 293425, 293426, 293427, 293428, 293429, 293430, 293431, 293432, 293433, 293434, 293435, 293436, 293437, 293438, 293439, 293440, 293441, 293442, 293443, 293444, 293445, 293446, 293447, 293448, 293449, 293450, 293451, 293452, 293453, 293454, 293455, 293456, 293457, 293458, 293459, 293460, 293461, 293462, 293463, 293464, 293465, 293466, 293467, 293468, 293469, 293470, 293471, 293472, 293473, 293474, 293475, 293476, 293477, 293478, 293479, 293480, 293481, 293482, 293483, 293484, 293485, 293486, 293487, 293488, 293489, 293490, 293491, 293492, 293493, 293494, 293495, 293496) c6( ) c7(bL, T, eR, mr, bfY, aw, ha, fN, cz, jh, ti, aj, v, jE, ci, nS, aY, XM, q, adb, J, hX, x, agd, cc, aE, aT, hD, jy, vc, hn, adm, K, A, ff, r, dq, yq, en, qa, Wt, ou, BU, HN, mg, cj, avl, k, ei, dl, XN, adh, abh); #c1(WARS) c2(XP_011535439) c3(tryptophan-tRNA ligase, cytoplasmic isoform X2) c4( ) c5(332468, 332469, 332470, 332471, 332472, 332473, 332474, 332475, 332476, 332477, 332478, 332479, 332480, 332481, 332482, 332483, 332484, 332485, 332486, 332487, 332488, 332489, 332490, 332491, 332492, 332493, 332494, 332495, 332496, 332497, 332498, 332499, 332500, 332501, 332502, 332503) c6( ) c7(bL, T, eR, mr, bfY, aw, ha, fN, cz, jh, ti, aj, v, jE, ci, nS, aY, XM, q, adb, J, hX, x, agd, cc, aE, aT, hD, jy, vc, hn, adm, K, A, ff, r, dq, yq, en, qa, Wt, ou, BU, HN, mg, cj, avl, k, ei, dL, XN, adh, abh); #c1(WARS2) c2(XP_011538796) c3(tryptophan-tRNA ligase, mitochondrial isoform X2) c4( ) c5(336543) c6( ) c7(hD, dq, aw, aE); #c1(WAS) c2(NP_000368) c3(wiskott-Aldrich syndrome protein) c4(8192) c5(8193, 8194, 8195, 8196, 8197, 8198) c6( ) c7(gV, T, dA, bs, bB, eb, afK, v, ai, br, eA, ds, aT, h, By, DU, ji, d, lx, hG, Cw, aW, oV, yL, fO, bys, ask, CF, gl, fe, aGd, cli, BG, eE, cm, jb, bw, ok, bQl, dB, bE, cUh, wR, zA, B); #c1(WAS) c2(XP_011542279) c3(wiskott-Aldrich syndrome protein isoform X1) c4(340881) c5(340882, 340883, 340884, 340885, 340886, 340887) c6( ) c7(gV, T, dA, bs, bB, eb, afK, v, ai, br, eA, ds, aT, h, By, DU, ji, d, lx, hG, Cw, aW, oV, yL, fO, bys, ask, CF, gl, fe, aGd, cOi, BG, BE, cm, jb, bw, ok, bQl, dB, bE, cUh, wR, zA, B); #c1(WASF1) c2(NP_001020106) c3(wiskott-Aldrich syndrome protein family member 1) c4(28146, 28147, 28148, 28149) c5(28150, 28151, 28152, 28153, 28154, 28155, 28156, 28157, 28158, 28159, 28160, 28161, 28162, 28163, 28164, 28165, 28166, 28167, 28168, 28169, 28/70, 28171) c6( ) c7(bv, T, sO, aW, bO, aT, r, cz, By, gf, bK, aj, nA, v, BG, he, ae, q, J, ad, aHY); #c1(WASF1) c2(XP_011534537) c3(wiskott-Aldrich syndrome protein family member 1 isoform X2) c4( ) c5(331059, 331060, 331061, 331062, 331063, 331064, 331065, 331066, 331067, 331068, 331069, 331070, 331071, 331072, 331073, 331074, 331075, 331076, 331077, 331078, 331079, 331080, 331081, 331082, 331083, 331084) c6( ) c7(bv, T, sO, aW, bO, al, r, cz, By, gf, bK, aj, nA, v, BG, he, ae, q, J, ad, aHY); #c1(WASF3) c2(NP_001278894) c3(wiskott-Aldrich syndrome protein family member 3 isoform 2) c4(110836) c5(110837, 110838, 110839, 110840, 110841, 110842) c6( ) c7(T, aw, bO, cz, aj, v, q, J, ad, Tg, ds, aql, aT, K, ae, CL, aW, r, BG, cm, fq, op, kP, vA, im); #c1(WASF3) c2(NP_006637) c3(wiskott-Aldrich syndrome protein family member 3 isoform 1) c4(166819) c5(166820, 166821, 166822, 166823, 166824, 166825, (66826) c6( ) c7(T, aw, bO, cz, aj, v, q, J, ad, Tg, ds, aql, aT, K, ae, CL, aW, r, BG, cm, fq, op, kP, vA, im); #c1(WASHI) c2(XP_011515960) c3(WAS protein family homolog 1 isoform X2) c4( ) c5(309093) c6( ) c7(T, cA, uJ); #c1(WASHI) c2(XP_011515962) c3(WAS protein family homolog 1 isoform X4) c4(309094) c5( ) c6( ) c7(T, cA, uJ); #c1(WASHI) c2(XP_011515966) c3(WAS protein family homolog 1 isoform X7) c4(309095, 309096) c5(309097, 309098, 309099, 309100, 309101, 309102, 309103, 309104, 309105, 309106, 309107, 309108) c6( ) c7(T, cA, uJ); #c1(WASHI) c2(XP_011515967) c3(WAS protein family homolog 1 isoform X8) c4(309109, 309110) c5(309111, 309112, 309113, 309114, 309115, 309116, 309117, 309118, 309119, 309120, 309121, 309122) c6( ) c7(T, cA, uJ); #c1(WASHI) c2(XP_011515968) c3(WAS protein family homolog 1 isoform X9) c4( ) c5(309123, 309124, 309125, 309126, 309127) c6( ) c7(T, cA, uJ); #c1(WASL) c2(NP_003932) c3(neural Wiskott-Aldrich syndrome protein) c4(142275, 142276) c5(142277, 142278, 142279, 142280, 142281, 142282, 142283, 142284, 142285, 142286, 142287, 142288) c6( ) c7(aW, ha, r, aT, vH, By, ah, BF, eA, BG, q); #c1(WBP1L) c2(NP_001077382) c3(WW domain binding protein (-like isoform 1 precursor) c4(37809) c5(37810, 37811, 37812, 37813, 37814, 37815) c6( ) c7(x, hF, k); #c1(WBP1L) c2(NP_060257) c3(WW domain binding protein 1-like isoform 2) c4(195095) c5(195096, 195097, 195098, 195099, 195100, 195101) c6( ) c7(x, hF, k); #c1(WBP2) c2(NP_036610) c3(WW domain-binding protein 2) c4(174802, 174803, 174804, 174805) c5(174806, 174807, 174808, 174809, 174810, 174811, 174812, 174813, 174814, 174815, 174816, 174817, 174818) c6( ) c7(aW, aT); #c1(WBP2) c2(XP_011522881) c3(WW domain-binding protein 2 isoform X1) c4( ) c5(317355, 317356, 317357, 317358, 317359) c6( ) c7(aW, aT); #c1(WBP2) c2(XP_011522882) c3(WW domain-binding protein 2 isoform X2) c4(317360) c5(317361, 317362, 317363, 317364, 317365, 317366, 317367, 317368, 317369, 317370, 317371, 317372) c6( ) c7(aW, aT); #c1(WBSCR17) c2(NP_071924) c3(putative polypeptide N-acetylgalactosaminyltransferase-like protein 3) c4(212336) c5(212337, 212338, 212339, 212340, 212341, 212342) c6( ) c7(af); #c1(WBSCR22) c2(NP_001189489) c3 (probable 18S rRNA (guanine-N(7))-methyltransferase isoform 1) c4(80257) c5(80258, 80259, 80260, 80261, 80262, 80263) c6( ) c7(T, alC, fD, aa); #c1(WBSCR22) c2(NP_059998) c3(probable 18S rRNA (guanine-N(7)-methyltransferase isoform 2) c4(193600) c5(193601, 193602, 193603, 193604, 193605, 193606) c6( ) c7(T, alC, fD, aa); #c1(WBSCR22) c2(XP_006715910) c3(probable 18S rRNA (guanine-N(7)-methyltransferase isoform X1) c4(289937) c5( ) c6( ) c7(T, alC, fD, aa); #c1(WBSCR22) c2(XP_011514080) c3(probable 18S rRNA (guanine-N(7)-methyltransferase isoform X2) c4( ) c5(306691, 306692, 306693, 306694, 306695, 306696) c6( ) c7(T, alC, fD, aa); #c1(WDFY2) c2(NP_443182) c3(WD repeat and FYVE domain-containing protein 2) c4(229747) c5(229748, 229749, 229750, 229751, 229752, 229753) c6( ) c7(T, m); #c1(WDFY4) c2(NP_065996) c3(WD repeat- and FYVE domain-containing protein 4) c4(206655, 206656, 206657, 206658, 206659) c5(206660, 206661, 206662, 206663, 206664, 206665, 206666, 206667, 206668, 206669, 206670, 206671, 206672, 206673, 206674, 206675, 206676, 206677, 206678, 206679, 206680, 206681, 206682, 206683, 206684, 206685, 206686, 206687, 206688, 206689, 206690, 206691, 206692, 206693, 206694, 206695, 206696, 206697, 206698, 206699, 206700) c6( ) c7(hH, fq, dL, eZ); #c1(WDFY4) c2(XP_011538293) c3(WD repeat- and FYVE domain-containing protein 4 isoform X5) c4(335953, 335954) c5(335955, 335956, 335957, 335958, 335959, 335960) c6( ) c7(hH, fq, dL, eZ); #c1(WDFY4) c2(XP_011538294) c3(WD repeat- and FYVE domain-containing protein 4 isoform X6) c4(335961, 335962) c5(335963, 335964, 335965, 335966, 335967, 335968, 335969) c6( ) c7(hH, fq, dL, eZ); #c1(WDHD1) c2(NP_001008397) c3(WD repeat and HMG-box DNA-binding protein 1 isoform 2) c4(23153, 23154) c5(23155, 23156, 23157, 23158, 23159, 23160, 23161, 23162, 23163, 23164) c6( ) c7(T, dD, eR, ea, hU, e, bO, cz, bB, n, aj, wo, afE, j, eq, asF, VV, eh, q, J, ad, aMB, fU, aCY, F, hd, tL, rw, gd, aT, h, K, aFm, ae, eL, qE, DA, A, aW, Y, ff, az, eH, r, gl, en, eF, t, bz, uP, qJ, bE, do, afd, cBv, wR, azg, m, R, bC, bt, B); #c1 (WDHD1) c2(NP_009017) c3(WD repeat and HMG-box DNA-binding protein 1 isoform 1) c4(170281) c5(170282, 170283, 170284, 170285, 170286, 170287, 170288) c6( ) c7(T, dD, eR, ea, hU, e, bO, cz, bB, n, aj, wo, afE, j, eq, asF, VV, eh, q, J, ad, aMB, FU, aCY, F, hd, tL, rw, gd, aT, h, K, aFm, ae, eL, qE, DA, A, aW, Y, ff, az, eH, r, gl, en, eF, t, bz, uP, qJ, bE, dO, afd, cBv, wR, azg, m, R, bC, bt, B); #c1(WDHD1) c2(XP_006720075) c3(WD repeat and HMG-box DNA-binding protein 1 isoform X1) c4(293196) c5(293197, 293198, 293199, 293200, 293201, 293202, 293203) c6( ) c7(T, dD, eR, ea, hU, e, bO, cz, bB, n, aj, wo, afE, j, eq, asF, W, eh, q, J, ad, aMB, fU, aCY, F, hd, tL, rw, gd, aT, h, K, aFm, ae, eL, qE, DA, A, aW, Y, ff, az, eL, r, gl, en, eF, t, bz, uP, qJ, bE, do, afd, cBv, wR, azg, m, R, bC, bt, B); #c1(WDPCP) c2(NP_056994) c3(WD repeat-containing and planar cell polarity effector protein fritz homolog isoform 2) c4(188376) c5(188377, 188378, 188379, 188380, 188381, 188382) c6( ) c7(wL, cli, Tp); #c1(WDR1) c2(NP_005103) c3(WD repeat-containing protein 1 isoform 2) c4(152888, 152889) c5(152890, 152891, 152892, 152893, 152894, 152895, 152896, 152897, 152898, 152899, 152900, 152901, 152902, 152903, 152904) c6( ) c7(Na, nn, nB, ea, Y, Ox, h, DU, bz, bA); #c1(WDR1) c2(NP_059830) c3(WD repeat-containing protein 1 isoform 1) c4(193385) c5(193386, 193387, 193388, 193389, 193390, 193391, 193392, 193393, 193394, 193395) c6( ) c7(Na, nn, nB, ea, Y, Ox, h, DU, bz, bA); #c1(WDR1) c2(XP_006714051) c3(WD repeat-containing protein 1 isoform X1) c4(287518) c5(287519, 287520, 287521, 287522, 287523, 287524, 287525) c6( ) c7(Na, nn, nB, ea, Y, Ox, h, DU, bz, bA); #c1(WDR11) c2(NP_060587) c3(WD repeat-containing protein 11) c4(196692) c5(196693, 196694, 196695, 196696, 196697, 196698) c6( ) c7(bM, T, jx, bwS, bcE, j, pd, eA, yn, aoH, NF, HD, bc, bHF, iU, hl, cUj, aF, Vk, pb, OW, nM, Lj, cj, UF, y, lB, hv); #c1(WDR12) c2(NP_060726) c3(ribosome biogenesis protein WDR(2) c4(197482) c5(197483, 197484, 197485, 197486, 197487, 197488) c6( ) c7(aw, j); #c1(WDR17) c2(NP_733828) c3(WD repeat-containing protein 17 isoform 1) c4(247192, 247193) c5(247194, 247195, 247196, 247197, 247198, 247199, 247200, 247201, 247202, 247203, 247204, 247205, 247206, 247207, 247208, 247209, 247210) c6( ) c7(aq); #c1(WDR17) c2(NP_851782) c3(WD repeat-containing protein 17 isoform 2) c4(254593) c5( ) c6( ) c7(aq); #c1(WDR17) c2(XP_011529883) c3(WD repeat-containing protein 17 isoform X1) c4( ) c5(325649, 325650, 325651, 325652, 325653, 325654, 325655) c6( ) c7(aq); #c1(WDR17) c2(XP_011529885) c3(WD repeat-containing protein 17 isoform X2) c4(325656) c5( ) c6( ) c7(aq); #c1(WDR19) c2(NP_079408) c3(WD repeat-containing protein 19) c4(217967, 217968, 217969) c5(217970, 217971, 217972, 217973, 217974, 217975, 217976, 217977, 217978, 217979, 217980, 217981, 217982, 217983, 217984, 217985, 217986, 217987, 217988, 217989, 217990, 217991, 217992, 217993) c6( ) c7(T, cUk, vT, cUm, bOx, fP, h, cUl, n, vm, yq, XU, fd, Vz, Sp, nC, ae, af, ad, aY, uQ); #c1(WDR20) c2(NP_001229344) c3(WD repeat-containing protein 20 isoform 5) c4(84254) c5(84255, 84256, 84257, 84258, 84259, 84260) c6( ) c7(T, ht, ml, amj, v, j, rc, q, bv, jz, xW, hu, bfS, yo, BO, ND, vT, LO, r, f, bF, bE, agl, Xu, B); #c1(WDR20) c2(NP_001229345) c3(WD repeat-containing protein 20 isoform 6) c4(84261, 84262) c5( ) c6( ) c7(T, ht, ml, amj, v, j, rc, q, bv, jz, xW, hu, bfS, yo, BO, ND, vT, LO, r, f, bF, bE, agl, Xu, B); #c1(WDR20) c2(NP_001229346) c3(WD repeat-containing protein 20 isoform 7) c4(84263, 84264) c5(84265, 84266, 84267, 84268, 84269, 84270) c6( ) c7(T, ht, ml, amj, v, j, rc, q, bv, jz, xW, hu, bfS, yo, BO, ND, vT, LO, r, f, bF, bE, agl, Xu, B); #c1(WDR20) c2(NP_001229347) c3(WD repeat-containing protein 20 isoform 8) c4(84271) c5(84272) c6( ) c7(T, ht, ml, amj, v, j, rc, q, bv, jz, xW, hu, bfS, yo, BO, ND, vT, LO, r, f, bF, bE, agl, Xu, B); #c1(WDR20) c2(NP_653175) c3(WD repeat-containing protein 20 isoform 2) c4(238944) c5(238945, 238946, 238947, 238948, 238949, 238950) c6( ) c7(T, ht, ml, amj, v, j, rc, q, bv, jz, xW, hu, bfS, yo, BO, ND, vT, LO, r, f, bF, bE, agl, Xu, B); #c1(WDR20) c2(NP_851808) c3(WD repeat-containing protein 20 isoform 1) c4(254594) c5(254595, 254596, 254597, 254598, 254599, 254600) c6( ) c7(T, ht, ml, amj, v, j, rc, q, bv, jz, xW, hu, bfS, yo, BO, ND, vT, LO, r, f, bF, bE, agl, Xu, B); #c1(WDR20) c2(NP_851825) c3(WD repeat-containing protein 20 isoform 3) c4(254615) c5(254616, 254617, 254618, 254619, 254620, 254621, 254622, 254623, 254624, 254625, 254626, 254627, 254628, 254629, 254630, 254631, 254632, 254633, 254634, 254635, 254636, 254637, 254638, 254639, 254640, 254641, 254642, 254643, 254644) c6( ) c7(T, ht, ml, amj, v, j, rc, q, bv, jz, xW, hu, bfS, yo, BO, ND, vT, LO, r, f, bF, bE, agl, Xu, B); #c1(WDR20) c2(XP_011535647) c3(WD repeat-containing protein 20 isoform X13) c4(332896) c5(332897, 332898, 332899, 332900, 332901, 332902) c6( ) c7(T, ht, ml, amj, v, j, rc, q, bv, jz, xW, hu, bfS, yo, BO, ND, vT, LO, r, f, bF, bE, agl, Xu, B); #c1(WDR26) c2(NP_001108585) c3(WD repeat-containing protein 26 isoform b) c4(43258) c5(43259) c6( ) c7(Y, uR, cU, wq, BF, K); #c1(WDR26) c2(NP_079436) c3(WD repeat-containing protein 26 isoform a) c4(218137, 218138) c5(218139, 218140, 218141, 218142, 218143, 218144, 218145, 218146, 218147, 218148, 218149, 218150) c6( ) c7(Y, uR, cl, wq, BF, K); #c1(WDR26) c2(XP_006711880) c3(WD repeat-containing protein 26 isoform X1) c4(286276) c5( ) c6( ) c7(Y, uR, cU, wq, BF, K); #c1(WDR26) c2(XP_011542582) c3(WD repeat-containing protein 26 isoform X3) c4( ) c5(341143, 341144, 341145, 341146, 341147) c6( ) c7(Y, uR, cU, wq, BF, K); #c1(WDR26) c2(XP_011542584) c3(WD repeat-containing protein 26 isoform X5) c4( ) c5(341148, 341149, 341150, 341151, 341152, 341153) c6( ) c7(Y, uR, cU, wq, BF, K); #c1(WDR31) c2(NP_001012361) c3(WD repeat-containing protein 31 isoform 1) c4(24904) c5(24905, 24906, 24907, 24908, 24909, 24910) c6( ) c7(aw, eu, e, j, ac); #c1(WDR31) c2(NP_660284) c3(WD repeat-containing protein 31 isoform 3) c4(240634, 240635) c5(240636, 240637, 240638, 240639, 240640, 240641, 240642) c6( ) c7(aw, eu, e, j, ac); #c1(WDR34) c2(NP_443076) c3(WD repeat-containing protein 34) c4(229276) c5(229277, 229278, 229279, 229280, 229281, 229282, 229283) c6( ) c7(bjc, cUn, bt, jj, bja); #c1(WDR34) c2(XP_011517481) c3(WD repeat-containing protein 34 isoform X1) c4(311219, 311220) c5(311221, 311222, 311223, 311224, 311225, 311226, 311227, 311228, 311229, 311230, 311231) c6( ) c7(bjc, cUn, bt, jj, bja); #c1(WDR35) c2(NP_001006658) c3(WD repeat-containing protein 35 isoform 1) c4(22331) c5(22332, 22333, 22334, 22335, 22336, 22337) c6( ) c7(gk, T, cUo, bOx, al, zG, cUp, atL); #c1(WDR35) c2(NP_065830) c3(WD repeat-containing protein 35 isoform 2) c4(205720) c5(205721, 205722, 205723, 205724, 205725, 205726, 205727) c6( ) c7(gk, T, cUo, bOx, al, zG, cUp, atL); #c1(WDR35) c2(XP_011531309) c3(WD repeat-containing protein 35 isoform X1) c4(327306) c5(327307, 327308, 327309, 327310, 327311) c6( ) c7(gk, T, cUo, bOx, al, zG, cUp, atL); #c1(WDR36) c2(NP_644810) c3(WD repeat-containing protein 36) c4(238565) c5(238566, 238567, 238568, 238569, 238570, 238571, 238572, 238573, 238574, 238575) c6( ) c7(HQ, bM, akc, pZ, dH, z, aw, v, cUq, K, dw, lJ); #c1(WDR37) c2(NP_054742) c3(WD repeat-containing protein 37) c4(177527, 177528, 177529) c5(177530, 177531, 177532, 177533, 177534, 177535, 177536, 177537, 177538, 177539, 177540, 177541, 177542, 177543, 177544, 177545, 177546, 177547, 177548, 177549, 177550, 177551) c6( ) c7(eR, dy, ac, cF); #c1(WDR4) c2(NP_387510) c3(tRNA (guanine-N(7)-)-methyltransferase non-catalytic subunit WDR4 isoform 1) c4(229013) c5(229014, 229015, 229016, 229017, 229018, 229019, 229020, 229021, 229022, 229023) c6( ) c7(cz); #c1(WDR43) c2(NP_055946) c3(WD repeat-containing protein 43) c4(184602) c5(184603, 184604, 184605, 184606, 184607, 184608) c6( ) c7(Xg); #c1(WDR45) c2(NP_001025067) c3(WD repeat domain phosphoinositide-interacting protein 4 isoform 2) c4(29299, 29300, 29301) c5(29302, 29303, 29304, 29305, 29306, 29307, 29308, 29309, 29310, 29311, 29312, 29313, 29314, 29315, 29316, 29317, 29318, 29319, 29320, 29321, 29322, 29323, 29324, 29325, 29326, 29327, 29328, 29329, 29330, 29331) c6( ) c7(aU, axa, cur, nA); #c1(WDR45) c2(NP_009006) c3(WD repeat domain phosphoinositide-interacting protein 4 isoform 1) c4(170198, 170199, 170200) c5(170201, 170202, 170203, 170204, 170205, 170206, 170207, 170208, 170209, 170210, 170211, 170212, 170213) c6( ) c7(aU, axa, cUr, nA); #c1(WDR45B) c2(NP_062559) c3(WD repeat domain phosphoinositide-interacting protein 3) c4(201950, 201951) c5(201952, 201953, 201954, 201955, 201956, 201957, 201958, 201959, 201960, 201961, 201962, 201963, 201964) c6( ) c7(nA); #c1(WDR46) c2(NP_005443) c3(WD repeat-containing protein 46 isoform 1) c4(156214, 156215, 156216, 156217, 156218, 156219) c5(156220, 156221, 156222, 156223, 156224, 156225, 156226, 156227, 156228, 156229, 156230, 156231, 156232, 156233, 156234, 156235, 156236, 156237, 156238, 156239, 156240, 156241, 156242, 156243, 156244, 156245, 156246, 156247, 156248, 156249, 156250, 156251, 156252, 156253, 156254, 156255, 156256, 156257, 156258, 156259, 156260, 156261, 156262, 156263, 156264, 156265, 156266, 156267, 156268) c6( ) c7(Y, eZ, aA); #c1(WDR48) c2(NP_001290331) c3(WD repeat-containing protein 48 isoform 2) c4(115178, 115179, 115180) c5(115181, 115182, 115183, 115184, 115185, 115186, 115187, 115188, 115189, 115190, 115191, 115192, 115193, 115194, 115195, 115196, 115197) c6( ) c7(os, XE, eH, jh, r, oZ, eF, nJ); #c1(WDR48) c2(NP_001290332) c3(WD repeat-containing protein 48 isoform 3) c4( ) c5(115198) c6( ) c7(os, XE, eH, jh, r, oZ, eF, nJ); #c1(WDR48) c2(NP_065890) c3(WD repeat-containing protein 48 isoform 1) c4(206079) c5(206080, 206081, 206082, 206083, 206084, 206085) c6( ) c7(os, XE, eH, jh, r, oZ, eF, nJ); #c1(WDR49) c2(NP_849146) c3(WD repeat-containing protein 49) c4(254280, 254281) c5(254282, 254283, 254284, 254285, 254286, 254287, 254288, 254289, 254290, 254291, 254292, 254293) c6( ) c7(j); #c1(WDR5) c2(XP_005272220) c3(WD repeat-containing protein 5 isoform X1) c4(283775) c5(283776, 283777, 283778, 283779, 283780, 283781, 283782, 283783, 283784, 283785) c6( ) c7(aW, ae, ad, aT); #c1(WDR55) c2(XP_005268526) c3(WD repeat-containing protein 55 isoform X1) c4(281876) c5(281877, 281878, 281879, 281880, 281881, 281882) c6( ) c7(aw, eu, e, j, ac); #c1(WDR60) c2(NP_060521) c3(WD repeat-containing protein 60) c4(196349) c5(196350, 196351, 196352, 196353, 196354, 196355) c6( ) c7(zG, cUs); #c1(WDR62) c2(NP_001077430) c3(WD repeat-containing protein 62 isoform) c4(37859, 37860, 37861) c5(37862, 37863, 37864, 37865, 37866, 37867) c6( ) c7(T, aii, afy, cUu, FB, aA, aF, nA, QK, cult, aij); #c1(WDR62) c2(NP_775907) c3(WD repeat-containing protein 62 isoform 2) c4(250656) c5(250657, 250658, 250659, 250660, 250661, 250662) c6( ) c7(T, aii, afy, cUu, FB, aA, aF, nA, QK, cut, aij); #c1(WDR62) c2(XP_005258866) c3(WD repeat-containing protein 62 isoform X2) c4(275837) c5( ) c6( ) c7(T, aii, afy, cUu, FB, aA, aF, nA, QK, cut, aij); #c1(WDR62) c2(XP_011525140) c3(WD repeat-containing protein 62 isoform X3) c4( ) c5(319579, 319580, 319581, 319582, 319583, 319584, 319585, 319586, 319587, 319588, 319589, 319590, 319591, 319592, 319593, 319594, 319595, 319596) c6( ) c7(T, aii, afy, cUu, FB, aA, aF, nA, QK, cUt, aij); #c1(WDR64) c2(NP_653226) c3(WD repeat-containing protein 64) c4(239192, 239193) c5(239194, 239195, 239196, 239197, 239198, 239199, 239200, 239201, 239202, 239203, 239204, 239205) c6( ) c7(V); #c1(WDR66) c2(NP_001171474) c3(WD repeat-containing protein 66 isoform 2) c4(70239) c5(70240, 70241, 70242, 70243, 70244, 70245) c6( ) c7(en, it, ff, afW); #c1(WDR66) c2(NP_653269) c3(WD repeat-containing protein 66 isoform 1) c4(239432) c5(239433, 239434, 239435, 239436, 239437, 239438) c6( ) c7(en, it, ff, afW); #c1(WDR7) c2(NP_056100) c3(WD repeat-containing protein 7 isoform 1) c4(185699) c5(185700, 185701, 185702, 185703, 185704, 185705, 185706, 185707, 185708, 185709, 185710, 185711) c6( ) c7(V, pN); #c1(WDR7) c2(NP_443066) c3(WD repeat-containing protein 7 isoform 2) c4(229187) c5(229188, 229189, 229190, 229191, 229192, 229193, 229194, 229195, 229196, 229197) c6( ) c7(V, pN); #c1(WDR7) c2(XP_005266731) c3(WD repeat-containing protein 7 isoform X3) c4(280289) c5( ) c6( ) c7(V, pN); #c1(WDR70) c2(NP_060504) c3(WD repeat-containing protein 70) c4(196281) c5(196282, 196283, 196284, 196285, 196286, 196287) c6( ) c7(hb); #c1(WDR72) c2(XP_011519737) c3(WD repeat-containing protein 72 isoform X1) c4(314406, 314407, 314408) c5(314409, 314410, 314411, 314412, 314413, 314414, 314415, 314416, 314417, 314418, 314419, 314420, 314421, 314422, 314423, 314424, 314425, 314426, 314427, 314428, 314429, 314430) c6( ) c7(eR, Ud, A, clv, ac); #c1(WDR74) c2(NP_060563) c3(WD repeat-containing protein 74) c4(196576, 196577, 196578) c5(196579, 196580, 196581, 196582, 196583, 196584, 196585, 196586, 196587, 196588, 196589, 196590, 196591, 196592) c6( ) c7(Y); #c1(WDR76) c2(NP_079184) c3(WD repeat-containing protein 76 isoform 1) c4(217339, 217340) c5(217341, 217342, 217343, 217344, 217345, 217346, 217347) c6( ) c7(q, CT, r); #c1(WDR76) c2(XP_005254737) c3(WD repeat-containing protein 76 isoform X1) c4( ) c5(273304, 273305, 273306, 273307, 273308) c6( ) c7(q, CT, r); #c1(WDR78) c2(XP_011540464) c3(WD repeat-containing protein 78 isoform X2) c4(338933) c5(338934, 338935, 338936, 338937, 338938, 338939) c6( ) c7(cj); #c1(WDR81) c2(NP_001157145) c3(WD repeat-containing protein 81 isoform 3) c4(62068, 62069) c5(62070, 62071, 62072, 62073, 62074, 62075) c6( ) c7(Sv, cUw, ky, adl); #c1(WDR81) c2(NP_001157281) c3(WD repeat-containing protein 81 isoform 1) c4(62134) c5(62135, 62136, 62137, 62138, 62139, 62140, 62141) c6( ) c7(Sv, cUw, ky, adl); #c1(WDR81) c2(XP_011521953) c3(WD repeat-containing protein 81 isoform X1) c4(316652, 316653, 316654) c5(316655, 316656, 316657, 316658, 316659, 316660, 316661, 316662, 316663, 316664, 316665, 316666, 316667, 316668, 316669, 316670, 316671, 316672, 316673, 316674, 316675, 316676, 316677) c6( ) c7(Sv, cUw, ky, adl); #c1(WDR83) c2(NP_115708) c3(WD repeat domain-containing protein 83) c4(223669) c5(223670, 223671, 223672, 223673, 223674, 223675, 223676, 223677, 223678, 223679) c6( ) c7(T, aF, aA); #c1(WDR86) c2(NP_001271190) c3(WD repeat-containing protein 86 isoform 3) c4( ) c5(103545, 103546, 103547, 103548, 103549) c6( ) c7(j); #c1(WDR86) c2(NP_938026) c3(WD repeat-containing protein 86 isoform 2) c4(259555) c5(259556, 259557, 259558, 259559, 259560, 259561) c6( ) c7(j); #c1(WDR86) c2(XP_005250046) c3(WD repeat-containing protein 86 isoform X4) c4(270059) c5(270060) c6( ) c7(j); #c1(WDR93) c2(NP_001271324) c3(WD repeat-containing protein 93 isoform 2) c4(103897) c5(103898, 103899, 103900, 103901, 103902, 103903) c6( ) c7(c); #c1(WDR93) c2(NP_064597) c3(WD repeat-containing protein 93 isoform 1) c4(203210) c5(203211, 203212, 203213, 203214, 203215, 203216) c6( ) c7(c); #c1(WEE1) c2(NP_001137448) c3(wee1-like protein kinase isoform 2) c4(55779) c5(55780, 55781, 55782, 55783, 55784) c6( ) c7(jf, T, yo, d, kP, eM, aW, Y, eH, aT, hR, n, K, eA, eF, cm, B, cp); #c1(WEE1) c2(NP_003381) c3(wee1-like protein kinase isoform 1) c4(138059) c5(138060, 138061, 138062, 138063, 138064, 138065, 138066) c6( ) c7(jf, T, yo, d, kP, eM, aW, Y, eH, aT, hR, n, K, eA, eF, cm, B, cp); #c1(WFDC1) c2(NP_001269395) c3(WAP four-disulfide core domain protein 1 isoform 1 precursor) c4(101561) c5(101562, 101563, 101564, 101565, 101566, 101567) c6( ) c7(T, eG, arH, Y, h, y, ae, ad, cp); #c1(WFDC2) c2(XP_011526797) c3(WAP four-disulfide core domain protein 2 isoform X1) c4(321075, 321076, 321077, 321078, 321079, 321080, 321081, 321082) c5(321083, 321084, 321085, 321086, 321087, 321088, 321089, 321090, 321091, 321092, 321093, 321094, 321095, 321096, 321097) c6(321098, 321099, 321100) c7(T, hL, oH, ci, m, cl, t, ad); #c1(WFS1) c2(NP_001139325) c3(wolframin) c4(59176, 59177) c5(59178, 59179, 59180, 59181, 59182, 59183, 59184, 59185, 59186, 59187, 59188, 59189, 59190, 59191, 59192, 59193) c6( ) c7(bL, tJ, aZ, Hm, aOH, bO, mS, aj, bS, dG, ct, v, aOG, aR, aqs, J, axC, av, mj, adG, mW, h, jy, bK, K, vd, an, mY, mV, he, mU, A, adF, Mz, cUA, Z, kC, mZ, bN, bCJ, mX, cA, cOy, mG, cUz, U, mg, Ei, awK, mf, ei, cUx, Bh, mT); #c1(WHSC1) c2(NP_015627) c3(histone-lysine N-methyltransferase NSD2 isoform 4) c4(171905, 171906, 171907, 171908) c5(171909, 171910, 171911, 171912, 171913, 171914, 171915, 171916, 171917, 171918, 171919, 171920, 171921, 171922, 171923, 171924, 171925, 171926, 171927, 171928, 171929, 171930, 171931, 171932, 171933, 171934, 171935, 171936, 171937) c6( ) c7(ck, ae, T, dP, x, aW, aOH, aT, fd, aeu, Ko, k, bz, ad, axC); #c1(WHSC1) c2(NP_579878) c3(histone-lysine N-methyltransferase NSD2 isoform 1) c4(234683) c5(234684, 234685, 234686, 234687, 234688, 234689, 234690, 234691, 234692, 234693, 234694, 234695, 234696, 234697, 234698, 234699, 234700, 234701, 234702, 234703, 234704, 234705, 234706) c6( ) c7(ck, ae, T, dP, x, aW, aOH, aT, fd, aeu, Ko, k, bz, ad, axC); #c1(WHSC1) c2(XP_006713977) c3 (histone-lysine N-methyltransferase NSD2 isoform X1) c4(287476, 287477) c5(287478, 287479, 287480, 287481, 287482, 287483, 287484, 287485, 287486, 287487) c6( ) c7(ck, ae, T, dP, x, aW, aOH, aT, fd, aeu, Ko, k, bz, ad, axC); #c1(WHSC1) c2(XP_011511861) c3(histone-lysine N-methyltransferase NSD2 isoform X4) c4(302587) c5(302588, 302589, 302590, 302591, 302592, 302593) c6( ) c7(ck, ae, T, dP, x, aW, aOH, al, fd, aeu, Ko, k, bz, ad, axC); #c1(WHSC1) c2(XP_011511862) c3(histone-lysine N-methyltransferase NSD2 isoform X5) c4( ) c5(302594) c6( ) c7(ck, ae, T, dP, x, aW, aOH, aT, fd, aeu, Ko, k, bz, ad, axC); #c1(WHSC1L1) c2(NP_060248) c3(histone-lysine N-methyltransferase NSD3 isoform short) c4(195007, 195008) c5(195009, 195010, 195011, 195012, 195013, 195014, 195015, 195016, 195017, 195018, 195019, 195020, 195021, 195022) c6( ) c7(av, T, ft, eM, aW, eL, aT, fd, aq, kp, bt, bz, eJ); #c1(WHSC1L1) c2(NP_075447) c3(histone-lysine N-methyltransferase NSD3 isoform long) c4(213866, 213867, 213868) c5(213869, 213870, 213871, 213872, 213873, 213874, 213875, 213876, 213877, 213878, 213879, 213880) c6( ) c7(av, T, ft, eM, aW, eL, aT, fd, aq, kp, bt, bz, eJ); #c1(WHSC1L1) c2(XP_005273604) c3(histone-lysine N-methyltransferase NSD3 isoform X3) c4(284707) c5(284708, 284709, 284710, 284711, 284712, 284713) c6( ) c7(av, T, ft, eM, aW, eL, aT, fd, aq, kp, bt, bz, eJ); #c1(WHSC1L1) c2(XP_005273605) c3(histone-lysine N-methyltransferase NSD3 isoform X4) c4(284714) c5(284715, 284716, 284717, 284718, 284719, 284720) c6( ) c7(av, T, ft, eM, aW, eL, aT, fd, aq, kp, bt, bz, eJ); #c1(WHSC1L1) c2(XP_011542864) c3(histone-lysine N-methyltransferase NSD3 isoform X1) c4(341474) c5(341475, 341476, 341477, 341478, 341479, 341480, 341481, 341482, 341483) c6( ) c7(av, T, ft, eM, aW, eL, aT, fd, aq, kp, bt, bz, eJ); #c1(WIFI) c2(NP_009122) c3(wnt inhibitory factor 1 precursor) c4(170872) c5(170873, 170874, 170875, 170876, 170877, 170878, 170879, (70880, (70881) c6( ) c7(bM, T, pf, if, lH, ht, aA, bB, n, fr, ci, qN, q, ad, ai, afb, av, ib, aE, bxn, aT, dL, jj, ZW, eL, YT, bA, qw, d, s, aW, Y, ev, fO, eH, iw, r, LZ, Pp, g, as, eF, apx, cm, bz, ahV, ck, es, bF, eM, iA, bE, gX, hR, y, bt, it, B, cn, vQ); #c1 (WIPF1) c2(NP_003378) c3(WAS/WASL-interacting protein family member 1) c4(137993, 137994, 137995, 137996, 137997) c5(137998, 137999, 138000, 138001, 138002, 138003, 138004, 138005, 138006, 138007, 138008, 138009, 138010, 138011, 138012, 138013, 138014, 138015, 138016, 138017, 138018, 138019, 138020, 138021, 138022, 138023, 138024, 138025, 138026, 138027, 138028, 138029, 138030, 138031, 138032, 138033, 138034, 138035, 138036) c6( ) c7(r, By, aGd, DU, BF, ey, BG, q, cUB); #c1(WIPF2) c2(NP_573571) c3(WAS/WASL-interacting protein family member 2) c4(234566, 234567) c5(234568, 234569, 234570, 234571, 234572, 234573) c6( ) c7(BG); #c1(WIPF2) c2(XP_011522715) c3(WAS/WASL-interacting protein family member 2 isoform X3) c4( ) c5(317242, 317243, 317244, 317245, 317246, 317247, 317248, 317249, 317250, 317251, 317252) c6( ) c7(BG); #c1(WIPF3) c2(NP_001073998) c3(WAS/WASL-interacting protein family member 3) c4(36879, 36880) c5(36881, 36882, 36883, 36884, 36885, 36886, 36887, 36888, 36889, 36890, 36891, 36892, 36893, 36894, 36895, 36896, 36897, 36898) c6( ) c7(vH, BG); #c1(WIPI1) c2(NP_060453) c3(WD repeat domain phosphoinositide-interacting protein 1) c4(196016, 196017) c5(196018, 196019, 196020, 196021, 196022, 196023, 196024) c6( ) c7(T, du); #c1(WIPI1) c2(XP_005257554) c3(WD repeat domain phosphoinositide-interacting protein 1 isoform X1) c4( ) c5(275262, 275263, 275264, 275265, 275266) c6( ) c7(T, du); #c1(WIPI2) c2(NP_001028690) c3(WD repeat domain phosphoinositide-interacting protein 2 isoform c) c4(30491) c5(30492, 30493, 30494, 30495, 30496, 30497) c6( ) c7(aw); #c1(WIPI2) c2(NP_001028691) c3(WD repeat domain phosphoinositide-interacting protein 2 isoform d) c4(30498) c5(30499, 30500, 30501, 30502, 30503, 30504) c6( ) c7(aw); #c1(WIPI2) c2(NP_001028692) c3(WD repeat domain phosphoinositide-interacting protein 2 isoform e) c4(30505, 30506) c5(30507, 30508, 30509, 30510, 30511, 30512, 30513, 30514, 30515, 30516, 30517, 30518) c6( ) c7(aw); #c1(WIPI2) c2(NP_056425) c3(WD repeat domain phosphoinositide-interacting protein 2 isoform a) c4(187545) c5(187546, 187547, 187548, 187549, 187550, 187551) c6( ) c7(aw); #c1(WIPI2) c2(NP_057087) c3(WD repeat domain phosphoinositide-interacting protein 2 isoform b) c4(188876) c5(188877, 188878, 188879, 188880, 188881, 188882) c6( ) c7(aw); #c1(WISP1) c2(NP_001191798) c3(WNT1-inducible-signaling pathway protein 1 isoform 3 precursor) c4(81806) c5(81807, 81808, 81809, 81810, 81811, 81812) c6( ) c7(bM, T, kh, cle, fG, cz, bB, ci, q, av, cF, jz, NS, aT, ji, qk, B, MD, aW, eH, r, aF, iV, eF, t, bz, LB, bE, X); #c1(WISP1) c2(NP_001191799) c3(WNT1-inducible-signaling pathway protein 1 isoform 4 precursor) c4(81813) c5(81814, 81815, 81816, 81817, 81818, 81819) c6( ) c7(bM, T, kh, cle, fG, cz, bB, ci, q, av, cF, jz, NS, aT, ji, qk, B, MD, aW, eH, r, aF, iV, eF, t, bz, LB, bE, X); #c1(WISP1) c2(NP_003873) c3(WNT1-inducible-signaling pathway protein 1 isoform 1 precursor) c4(141698) c5(141699, 141700, 141701, 141702, 141703, 141704) c6( ) c7(bM, T, kh, cle, fG, cz, bB, ci, q, av, cF, jz, NS, aT, ji, qk, B, MD, aW, eH, r, aF, iV, eF, t, bz, LB, bE, X); #c1(WISP1) c2(NP_543028) c3(WNT1-inducible-signaling pathway protein 1 isoform 2 precursor) c4(233294) c5(233295, 233296, 233297, 233298, 233299, 233300) c6( ) c7(bM, T, kh, cle, fG, cz, bB, ci, q, av, cF, jz, NS, aT, ji, qk, B, MD, aW, eH, r, aF, iV, eF, t, bz, LB, bE, X); #c1(WISP1) c2(XP_006716743) c3(WNT1-inducible-signaling pathway protein 1 isoform X2) c4(290446) c5( ) c6( ) c7(bM, T, kh, cle, fG, cz, bB, ci, q, av, cF, jz, NS, aT, ji, qk, B, MD, aW, eH, r, aF, iV, eF, t, bz, LB, bE, X); #c1(WISP1) c2(XP_011515661) c3(WNT1-inducible-signaling pathway protein 1 isoform X3) c4( ) c5(308508, 308509, 308510, 308511, 308512, 308513) c6( ) c7(bM, T, kh, cle, fG, cz, bB, ci, q, av, cF, jz, NS, aT, ji, qk, B, MD, aW, eH, r, aF, iV, eF, t, bz, LB, bE, X); #c1(WISP2) c2(XP_005260660) c3(WNT1-inducible-signaling pathway protein 2 isoform X1) c4(276786, 276787) c5(276788, 276789, 276790, 276791, 276792, 276793, 276794, 276795, 276796, 276797, 276798, 276799, 276800, 276801, 276802, 276803, 276804) c6( ) c7(eW, T, eJ, aE, aW, vA, r, aT, hR, Ja, g, im, q, op); #c1(WISP3) c2(NP_003871) c3(WNT1-inducible-signaling pathway protein 3 isoform 1 precursor) c4(141666) c5(141667, 141668, 141669, 141670, 141671, 141672, 141673, 141674, 141675, 141676, 141677, 141678, 141679, 141680, 141681, 141682, 141683, 141684, 141685, 141686, 141687, 141688, 141689, 141690, 141691, 141692, 141693, 141694, 141695, 141696, 141697) c6( ) c7(gh, T, kh, e, aRm, cl, ci, zU, Ll, q, o, ad, LH, ai, aE, ec, aT, vw, Wr, LG, ae, rv, B, MD, aW, r, LJ, dr, cA, ig, zG, y, X); #c1(WISP3) c2(NP_937882) c3(WNT1-inducible-signaling pathway protein 3 isoform 3) c4(259388, 259389, 259390, 259391) c5(259392, 259393, 259394, 259395, 259396, 259397, 259398, 259399, 259400, 259401) c6( ) c7(gh, T, kh, e, aRm, cl, ci, zU, Ll, q, o, ad, LH, ai, aE, ec, aT, vw, Wr, LG, ae, rv, B, MD, aW, r, LJ, dr, cA, ig, zG, y, X); #c1(WISP3) c2(XP_011534525) c3(WNT1-inducible-signaling pathway protein 3 isoform X4) c4( ) c5(331011, 331012, 331013, 331014, 331015, 331016, 331017, 331018) c6( ) c7(gh, T, kh, e, aRm, cl, ci, zu, Ll, q, o, ad, LH, ai, aE, ec, aT, vw, Wr, LG, ae, rv, B, MD, aW, r, LJ, dr, cA, ig, zG, y, X); #c1(WLS) c2(XP_011540493) c3(protein wntless homolog isoform X1) c4(338980) c5(338981, 338982, 338983, 338984, 338985, 338986) c6( ) c7(T, ht, jh, aA, bB, KM, fd, fr, ci, KO, hF, q, ad, eA, x, av, iu, ft, ib, aE, dV, aT, h, dL, K, hu, ae, bc, eL, bA, jf, gc, kF, d, iZ, hG, aW, Y, KK, ff, cr, r, aq, Ph, aF, en, hf, cl, t, kC, bz, ho, dr, as, cm, ck, iR, eM, bE, gX, cj, hL, m, rH, bt, B); #c1(WNK1) c2(NP_001171914) c3(serine/threonine-protein kinase WNK1 isoform 4) c4(72019) c5(72020, 72021, 72022, 72023, 72024, 72025) c6( ) c7(oO, bM, T, os, ahS, if, e, al, bEy, eb, fd, IR, ci, bv, aE, K, eG, aW, Fe, az, asD, bWB, g, eE, cm, bw, axG, nB, cj, bqQ, m, tp, ht, aj, BF, qN, eW, md, kH, cU, pi, bT, gi, fe, kp, dB, bjx, tl, it, B, aOo, L, oe, aA, cz, C, bX, V, q, J, eA, eJ, yg, cUC, aT, ae, af, eu, aAi, jf, nO, Y, r, en, t, f, cmk, pb, iK, ck, iM, tW, c, ac, ig, hr, cFC, dy, bt, Pt, qb, aFa, oZ, dG, fu, ad, XB, dP, hd, cbH, azq, ji, akS, azR, gj, d, je, ff, xs, eZ, aF, iV, qa, DF, bF, cqP, y, kS, X, brt); #c1(WNK1) c2(NP_055638) c3(serine/threonine-protein kinase WNK1 isoform 2) c4(182432) c5(182433, 182434, 182435, 182436, 182437, 182438) c6( ) c7(oO, bM, T, os, ahS, if, e, al, bEy, eb, fd, lR, ci, bv, aE, K, eG, aW, Fe, az, asD, bWB, g, eE, cm, bw, axG, nB, cj, bqQ, m, tp, ht, aj, BF, qN, eW, md, kH, cU, pi, bT, gi, fe, kp, dB, bjx, tl, it, B, aOo, L, oe, aA, cz, C, bX, V, q, J, eA, eJ, yg, cUC, aT, ae, af, eu, aAi, jf, n, Y, r, en, t, f, cmk, pb, iK, ck, iM, tW, c, ac, ig, hr, cFC, dy, bt, Pt, qb, aFa, oZ, dG, fu, ad, XB, dP, hd, cbH, azq, ji, akS, azR, gj, d, je, ff, xs, eZ, aF, iV, qa, DF, bF, cqP, y, kS, X, brt); #c1(WNK1) c2(NP_061852) c3(serine/threonine-protein kinase WNK1 isoform 1) c4(200785, 200786, 200787) c5(200788, 200789, 200790, 200791, 200792, 200793, 200794, 200795, 200796, 200797, 200798, 200799, 200800, 200801, 200802, 200803, 200804, 200805) c6( ) c7(oO, bM, T, os, ahS, if, e, al, bEy, eb, fd, lR, ci, bv, aE, K, eG, aW, Fe, az, asD, bWB, g, eE, cm, bw, axG, nB, cj, bqQ, m, tp, ht, aj, BF, qN, eW, md, kH, cU, pi, bT, gi, fe, kp, dB, bjx, tl, it, B, aOo, L, De, aA, cz, C, bX, V, q, J, eA, eL, yg, cUC, aT, ae, af, eu, aAi, jf, nO, Y, r, en, t, f, cmk, pb, iK, ck, iM, tW, c, ac, ig, hr, cFC, dy, bt, Pt, qb, aFa, oZ, dG, fu, ad, XB, dP, hd, cbH, azq, ji, akS, azR, gj, d, je, ff, xs, eZ, aF, iV, qa, DF, bF, cqP, y, kS, X, brt); #c1(WNK1) c2(XP_011519300) c3(serine/threonine-protein kinase WNK1 isoform X2) c4(313946) c5( ) c6( ) c7(oO, bM, T, os, ahS, if, e, al, bEy, eb, fd, lR, ci, bv, aE, K, eG, aW, Fe, az, asD, bWB, g, eE, cm, bw, axG, nB, cj, bqQ, m, tp, ht, aj, BF, qN, eW, md, kH, cU, pi, bT, gi, fe, kp, dB, bjx, tl, it, B, aOo, L, oe, aA, cz, C, bX, V, q, J, eA, eJ, yg, cUC, aT, ae, af, eu, aAi, jf, nO, Y, r, en, t, f, cmk, pb, iK, ck, iM, tW, c, ac, ig, hr, cFC, dy, bt, Pt, qb, aFa, oZ, dG, fu, ad, XB, dP, hd, cbH, azq, ji, akS, azR, gj, d, je, ff, xs, eZ, aF, iV, qa, DF, bF, cqP, y, kS, X, brt); #c1(WNK1) c2(XP_011519310) c3(serine/threonine-protein kinase WNK1 isoform X13) c4(313947) c5(313948, 313949, 313950, 313951, 313952, 313953, 313954) c6( ) c7(oO, bM, T, os, ahS, if, e, al, bEy, eb, fd, lR, ci, bv, aE, K, eG, aW, Fe, az, asD, bWB, g, eE, cm, bw, axG, nB, cj, bqQ, m, tp, ht, aj, BF, qN, eW, md, kH, cU, pi, bT, gi, fe, kp, dB, bjx, tl, it, B, aOo, L, De, aA, cz, C, bX, V, q, J, eA, eJ, yg, cUC, aT, ae, af, eu, aAi, jf, nO, Y, r, en, t, f, cmk, pb, iK, ck, iM, tW, c, ac, ig, hr, cFC, dy, bt, Pt, qb, aFa, oZ, dG, fu, ad, XB, dP, hd, cbH, azq, ji, akS, azR, gj, d, je, ff, xs, eZ, aF, iV, qa, DF, bF, cqP, y, kS, X, brt); #c1(WNK1) c2(XP_011519311) c3(serine/threonine-protein kinase WNK1 isoform X14) c4(313955) c5(313956, 313957, 313958, 313959, 313960, 313961, 313962, 313963, 313964, 313965, 313966) c6( ) c7(oO, bM, T, os, ahS, if, e, al, bEy, eb, fd, lR, ci, bv, aE, K, eG, aW, Fe, az, asD, bWB, g, BE, cm, bw, axG, nB, cj, bqQ, m, tp, ht, aj, BF, qN, eW, md, kH, cU, pi, bT, gi, fe, kp, dB, bjx, tl, it, B, aOo, L, De, aA, cz, C, bX, V, q, J, eA, eJ, yg, cUC, aT, ae, af, eu, aAi, jf, n, Y, r, en, t, f, cmk, pb, iK, ck, iM, tW, c, ac, ig, hr, cFC, dy, bt, Pt, qb, aFa, oZ, dG, fu, ad, XB, dP, hd, cbH, azq, ji, akS, azR, gj, d, je, ff, xs, eZ, aF, iV, qa, DF, bF, cqP, y, kS, X, brt); #c1(WNK2) c2(NP_001269323) c3(serine/threonine-protein kinase WNK2 isoform 1) c4(101125, 101126) c5(101127, 101128, 101129, 101130, 101131, 101132, 101133, 101134, 101135, 101136, 101137, 101138) c6( ) c7(ar, T, d, Dy, ac, dB, Y, kp, dy, LV, eA, ip); #c1(WNK2) c2(XP_005252197) c3(serine/threonine-protein kinase WNK2 isoform X12) c4(271594) c5(271595, 271596, 271597, 271598, 271599, 271600) c6( ) c7(ar, T, d, Dy, ac, dB, Y, kp, dy, LV, eA, ip); #c1(WNK2) c2(XP_005252198) c3(serine/threonine-protein kinase WNK2 isoform X1) c4(271601) c5(271602, 271603, 271604, 271605, 271606, 271607, 271608) c6( ) c7(ar, T, d, Dy, ac, dB, Y, kp, dy, LV, eA, ip); #c1(WNK2) c2(XP_011517229) c3(serine/threonine-protein kinase WNK2 isoform X3) c4(310812) c5(310813) c6( ) c7(ar, T, d, Dy, ac, dB, Y, kp, dy, LV, eA, ip); #c1(WNK2) c2(XP_011517235) c3(serine/threonine-protein kinase WNK2 isoform X10) c4(310814) c5(310815, 310816, 310817, 310818, 310819) c6( ) c7(ar, T, d, Dy, ac, dB, Y, kp, dy, LV, eA, ip); #c1(WNK2) c2(XP_011517242) c3(serine/threonine-protein kinase WNK2 isoform X18) c4( ) c5(310820, 310821, 310822, 310823, 310824, 310825, 310826, 310827, 310828) c6( ) c7(ar, T, d, Dy, ac, dB, Y, kp, dy, LV, eA, ip); #c1(WNK3) c2(NP_065973) c3(serine/threonine-protein kinase WNK3 isoform 1) c4(206557) c5(206558, 206559, 206560, 206561, 206562, 206563, 206564, 206565, 206566, 206567, 206568, 206569, 206570, 206571, 206572, 206573, 206574, 206575, 206576) c6( ) c7(nf, bN, bqN); #c1(WNK3) c2(XP_006724656) c3(serine/threonine-protein kinase WNK3 isoform X1) c4(296178, 296179) c5(296180, 296181, 296182, 296183, 296184, 296185) c6( ) c7(nf, bN, bqN); #c1(WNK4) c2(NP_115763) c3(serine/threonine-protein kinase WNK4) c4(223852) c5(223853, 223854, 223855, 223856, 223857, 223858) c6( ) c7(qa, bA, jf, ahS, cqP, Fe, gC, cz, cUD, tl, afV, bWB, K, cUE, aY, dr, aOo); #c1(WNK4) c2(XP_005257652) c3(serine/threonine-protein kinase WNK4 isoform X1) c4( ) c5(275319) c6( ) c7(qa, bA, jf, ahS, cqP, Fe, gC, cz, cUD, tl, afV, bWB, K, cUE, aY, dr, aOo); #c1(WNK4) c2(XP_006722085) c3(serine/threonine-protein kinase WNK4 isoform X10) c4(294605) c5(294606, 294607, 294608, 294609, 294610) c6( ) c7(qa, bA, jf, ahS, cqP, Fe, gC, cz, cUD, tl, afV, bWB, K, cUE, aY, dr, aOo); #c1(WNK4) c2(XP_011523437) c3(serine/threonine-protein kinase WNK4 isoform X8) c4(3/7964) c5(317965, 317966, 317967, 317968, 317969, 317970) c6( ) c7(qa, bA, jf, ahS, cqP, Fe, gC, cz, cUD, tl, afV, bWB, K, cUE, aY, dr, aOo); #c1(WNT1) c2(NP_005421) c3(proto-oncogene Wnt-1 precursor) c4(155981, 155982) c5(155983, 155984, 155985, 155986, 155987, 155988, 155989, 155990, 155991, 155992, 155993, 155994) c6( ) c7(cUF, bM, T, fG, ht, aA, n, cUG, ci, q, ad, eA, hX, Na, ft, aE, afQ, aT, h, zp, rz, ae, bA, jf, B, MD, qt, aW, Y, ff, r, fe, aq, af, en, bw, QA, LB, iO, bF, fT, bP, pP, U, aqH, y, bt, ra, X); #c1 (WNT1DA) c2(NP_079492) c3(protein Wnt-10a precursor) c4(218352) c5(218353, 218354, 218355, 218356, 218357, 218358) c6( ) c7(T, bjB, ht, aA, Hn, fr, aml, q, amH, cUK, bjv, bjA, hu, bjy, cUI, az, r, aF, cUH, hk, dy, cUJ); #c1(WNT10B) c2(NP_003385) c3(protein Wnt-10b precursor) c4(138103) c5(138104, 138105, 138106, 138107, 138108, 138109, 138110, 138111) c6( ) c7(T, kP, arA, c, yE, aW, if, eF, eH, aT, aA, v, oH, ci, qN, cl, cUL, t, aF, aRR); #c1(WNT10B) c2(XP_011537024) c3 (protein Wnt-10b isoform X1) c4( ) c5(334780) c6( ) c7(T, kP, arA, c, yE, aW, if, eF, eH, aT, aA, v, oH, ci, qN, cl, cUL, t, aF, aRR); #c1(WNT10B) c2(XP_011537025) c3(protein Wnt-10b isoform X2) c4(334781, 334782) c5(334783, 334784, 334785, 334786, 334787, 334788, 334789) c6( ) c7(T, kP, arA, c, yE, aW, if, eF, eH, al, aA, v, oH, ci, qN, cl, cUL, t, aF, aRR); #c1(WNT10B) c2(XP_011537026) c3(protein Wnt-10b isoform X3) c4( ) c5(334790, 334791, 334792, 334793, 334794, 334795, 334796, 334797) c6( ) c7(T, kP, arA, c, yE, aW, if, eF, eH, al, aA, v, oH, ci, qN, cl, cUL, t, aF, aRR); #c1(WNT11) c2(NP_004617) c3(protein Wnt-Il precursor) c4(148202, 148203) c5(148204, 148205, 148206, 148207, 148208, 148209, 148210, 148211, 148212, 148213, 148214, 148215) c6( ) c7(oO, T, EQ, bB, n, hy, fr, bX, cd, q, ad, Na, gH, aT, bma, Nc, ae, B, BN, aW, r, g, ck, bF, ac, bE, Hb, bt, ne); #c1(WNT16) c2(NP_057 (71) c3(protein Wnt-16 isoform 2) c4(189292, 189293) c5(189294, 189295, 189296, 189297, 189298, 189299, 189300, 189301, 189302, 189303, 189304, 189305, 189306, 189307, 189308) c6(189309, 189310, 189311) c7(ck, ou, e, v, afl, bD, agk, bA); #c1(WNT16) c2(NP_476509) c3(protein Wnt-16 isoform 1 precursor) c4(230678) c5(230679, 230680, 230681, 230682, 230683, 230684, 230685, 230686) c6( ) c7(ck, ou, e, v, afl, bD, agk, bA); #c1(WNT2) c2(NP_003382) c3(protein Wnt-2 precursor) c4(138067) c5(138068, 138069, 138070, 138071, 138072, 138073) c6( ) c7(T, if, e, aA, ci, q, eA, bv, jz, ib, aE, oW, aT, yu, cUM, Dz, aUQ, s, Ar, nO, aW, Y, r, aF, cl, t, pb, bN, gX, ao); #c1(WNT2B) c2(NP_001278809) c3(protein Wnt-2b isoform 3) c4( ) c5(110147, 110148, 110149, 110150, 110151, 110152, 110153) c6(110154, 110155) c7(T, BN, dB, if, eD, ht, Be, r, aA, Hn, lo, hy, bK, aF, qN, t, Bd, ne); #c1(WNT3) c2(NP_110380) c3 (proto-oncogene Wnt-3 precursor) c4(219812) c5(219813, 219814, 219815, 219816, 219817, 219818) c6( ) c7(T, aw, aA, CH, bB, ci, cUN, Na, AW, bKk, oW, a, bma, Nc, an, aa, Y, aF, hp, bz, pb, ac, eM, ig, bE, Ny, dy, act, B); #c1(WNT3A) c2(NP_149122) c3(protein Wnt-3a precursor) c4(227295) c5(227296, 227297, 227298, 227299, 227300, 227301) c6( ) c7(IH, fd, cR, bB, fr, q, eA, ad, dP, x, Na, zw, fm, aT, Nc, K, ae, d, aW, Y, r, t, ck, iA, bE, m, gT, B); #c1(WNT3A) c2(XP_011542622) c3 (protein Wnt-3a isoform X2) c4(341181) c5(341182, 341183, 341184, 341185, 341186, 341187) c6( ) c7(IH, fd, cR, bB, fr, q, eA, ad, dP, x, Na, zw, fm, aT, Nc, K, ae, d, aW, Y, r, t, ck, iA, bE, m, gT, B); #c1(WNT4) c2(XP_011539899) c3 (protein Wnt-4 isoform X1) c4( ) c5(338164) c6( ) c7(T, UH, eR, xo, v, aY, cUO, ad, dP, iI, BJ, aT, xp, uV, Dz, Ur, UL, aW, ff, en, hf, cl, zZ, t, iV, ck, bfP, hR, E, at, bxG, m, Hb, dy, fD); #c1(WNT4) c2(XP_011539900) c3(protein Wnt-4 isoform X2) c4( ) c5(338165, 338166, 338167, 338168, 338169) c6( ) c7(T, UH, eR, xo, v, aY, cUD, ad, dP, iI, BJ, aT, xp, uV, Dz, Ur, UL, aW, ff, en, hf, cl, zZ, t, iV, ck, bfP, hR, E, at, bxG, m, Hb, dy, fD); #c1(WNT4) c2(XP_011539901) c3(protein Wnt-4 isoform X3) c4(338170, 338171, 338172) c5(338173, 338174, 338175, 338176, 338177, 338178, 338179, 338180, 338181, 338182, 338183, 338184, 338185, 338186, 338187, 338188, 338189, 338190) c6( ) c7(T, UH, eR, xo, v, aY, cUO, ad, dP, iI, BJ, aT, xp, uV, Dz, Ur, UL, aW, ff, en, hf, cl, zZ, t, iV, ck, bfP, hR, E, at, bxG, m, Hb, dy, fD); #c1(WNT5A) c2(NP_001243034) c3(protein Wnt-Sa isoform 2 precursor) c4( ) c5(89639, 89640, 89641, 89642, 89643, 89644, 89645, 89646, 89647, 89648, 89649, 89650, 89651, 89652, 89653, 89654, 89655) c6( ) c7(T, dD, fM, aC, ea, if, jx, aA, bB, fd, v, eq, ci, C, AD, q, ad, er, hX, eA, eJ, av, x, Na, aE, bW, kH, aT, bma, Nc, dL, in, H, KV, ae, eL, dx, cp, Ur, de, qw, jf, cml, d, je, aW, Y, ff, az, cr, r, aF, VI, en, hf, kk, t, cM, bz, g, LB, ck, sS, nM, iM, c, nB, dB, bE, eM, iT, X, ajd, cfv, m, MC, y, bt, B, L); #c1(WNT5A) c2(NP_003383) c3(protein Wnt-5a isoform 1) c4(138074, 138075, 138076, 138077, 138078) c5(138079, 138080, 138081, 138082, 138083, 138084, 138085, 138086, 138087, 138088, 138089, 138090, 138091, 138092, 138093, 138094, 138095) c6( ) c7(T, dD, fM, aC, ea, if, jx, aA, bB, fd, v, eq, ci, C, AO, q, ad, er, hX, eA, eJ, av, x, Na, aE, bW, kH, aT, bma, Nc, dL, in, H, KV, ae, eL, dx, cp, Ur, de, qw, jf, cml, d, je, aW, Y, ff, az, cr, r, aF, VI, en, hf, kk, t, cM, bz, g, LB, ck, sS, nM, iM, c, nB, dB, bE, eM, iT, X, ajd, cfv, m, MC, y, bt, B, L); #c1(WNT5B) c2(NP_110402) c3 (protein Wnt-5b precursor) c4(220066) c5(220067, 220068, 220069, 220070, 220071, 220072, 220073, 220074, 220075, 220076, 220077, 220078, 220079, 220080) c6( ) c7(bL, aW, vA, eH, qK, ff, aT, v, en, eF, op); #c1(WNT6) c2(NP_006513) c3(protein Wnt-6 precursor) c4(165921) c5(165922, 165923, 165924, 165925, 165926, 165927) c6( ) c7(aW, az, aA, aT, dL, aF, eL); #c1(WNT7A) c2(NP_004616) c3(protein Wnt-7a precursor) c4(148195) c5(148196, 148197, 148198, 148199, 148200, 148201) c6( ) c7(T, oe, fr, PJ, dP, XW, av, x, ib, Na, aT, Nc, cUP, bK, Ur, xs, la, cUQ, Z, t, bz, op, atb, ck, bfP, eM, go, vA, k, m); #c1(WNT7B) c2(NP_478679) c3(protein Wnt-7b precursor) c4(230986, 230987) c5(230988, 230989, 230990, 230991, 230992, 230993, 230994, 230995, 230996, 230997, 230998, 230999, 231000, 231001, 231002, 231003, 231004, 231005, 231006, 231007, 231008, 231009, 231010) c6(231011) c7(ae, T, aE, Dh, aW, eL, Vr, aT, aA, dL, aF, Dz, bz, ad); #c1(WNT7B) c2(XP_011528668) c3(protein Wnt-7b isoform X1) c4(323931, 323932, 323933) c5(323934, 323935, 323936, 323937, 323938, 323939, 323940, 323941, 323942, 323943, 323944, 323945, 323946, 323947, 323948, 323949) c6( ) c7(ae, T, aE, Dh, aW, eL, Vr, aT, aA, dL, aF, Dz, bz, ad); #c1(WNT8A) c2(NP_490645) c3(protein Wnt-8a isoform 3 precursor) c4(231019) c5(231020, 231021, 231022, 231023, 231024, 231025, 231026, 231027, 231028) c6(231029, 231030) c7(Na, wL, aA, bma, Nc, aF); #c1(WNT8B) c2(NP_003384) c3(protein Wnt-8b precursor) c4(138096) c5(138097, 138098, 138099, 138100, 138101, 138102) c6( ) c7(YL, aA, r, aF, ci, g, q); #c1(WNT9A) c2(NP_003386) c3(protein Wnt-9a precursor) c4(138112) c5(138113, 138114, 138115, 138116, 138117, 138118, 138119, 138120) c6(138121, 138122) c7(bF, T, hn, aT); #c1(WNT9B) c2(NP_003387) c3(protein Wnt-9b precursor) c4(138123) c5(138124, 138125, 138126, 138127, 138128, 138129, 138130, 138131) c6(138132, 138133) c7(Na, bfP, lo, bma, Nc); #c1(WNT9B) c2(XP_005257694) c3(protein Wnt-9b isoform X2) c4(275340) c5(275341, 275342, 275343, 275344, 275345, 275346, 275347, 275348) c6(275349, 275350) c7(Na, bfP, lo, bma, Nc); #c1(WNT9B) c2(XP_011523480) c3(protein Wnt-9b isoform X1) c4(318006) c5(318007, 318008, 318009, 318010, 318011, 318012, 318013, 318014) c6(318015, 318016) c7(Na, bfP, lo, bma, Nc); #c1(WRAP53) c2(NP_001137462) c3(telomerase Cajal body protein 1) c4(55851, 55852) c5(55853, 55854, 55855, 55856, 55857, 55858, 55859, 55860, 55861, 55862, 55863, 55864, 55865, 55866, 55867, 55868, 55869, 55870) c6( ) c7(sb, GI, T, asi, cUR, aW, ht, aT, at, hy, dL, Gw, Gu); #c1(WRAP53) c2(XP_011522254) c3 (telomerase Cajal body protein 1 isoform X1) c4( ) c5(316890, 316891, 316892, 316893, 316894, 316895) c6( ) c7(sb, GI, T, asi, cUR, aW, ht, aT, at, hy, dL, Gw, Gu); #c1(WRB) c2(NP_001139690) c3(tail-anchored protein insertion receptor WRB isoform 2) c4(59743) c5(59744, 59745, 59746, 59747, 59748, 59749, 59750, 59751, 59752) c6( ) c7(sc, T, aA, j, ci, q, ad, eA, dP, aT, aW, Y, ff, r, Ej, aF, en, cm, f, t, bz, ck, Dy, m, B); #c1(WRB) c2(NP_004618) c3(tail-anchored protein insertion receptor WRB isoform 1) c4(148216, 148217) c5(148218, 148219, 148220, 148221, 148222, 148223, 148224, 148225, 148226, 148227, 148228, 148229) c6( ) c7(sc, T, aA, j, ci, q, ad, eA, dP, aT, aW, Y, ff, r, Ej, aF, en, cm, f, t, bz, ck, Dy, m, B); #c1(WRB) c2(XP_011528046) c3(tail-anchored protein insertion receptor WRB isoform X2) c4( ) c5(323146) c6( ) c7(sc, T, aA, j, ci, q, ad, eA, dP, aT, aW, Y, ff, r, Ej, af, en, cm, f, t, bz, ck, Dy, m, B); #c1(WRN) c2(NP_000544) c3(Werner syndrome ATP-dependent helicase) c4(12589) c5(12590, 12591, 12592, 12593, 12594, 12595) c6( ) c7(bL, T, os, dD, dA, aEj, hV, cUS, ea, aw, ht, iW, ew, jh, bB, aj, v, j, ci, bX, oZ, eq, V, q, J, eA, asi, LL, MR, QJ, rC, yg, aT, h, dL, K, hu, ask, dx, alK, wr, iU, Jl, aW, aAu, Of, r, Og, La, lk, DL, pb, dr, iK, Qi, ac, at, bon, bC, ei, iu); #c1(WRN) c2(XP_011542942) c3(Werner syndrome ATP-dependent helicase isoform X2) c4( ) c5(341637) c6( ) c7(bL, T, os, dD, dA, aEj, hV, cUS, ea, aw, ht, iW, ew, jh, bB, aj, v, j, ci, bX, oZ, eq, V, q, J, eA, asi, LL, MR, QJ, rC, yg, aT, h, dL, K, hu, ask, dx, alK, wr, iU, Jl, aW, aAu, Of, r, Og, La, lk, DL, pb, dr, iK, Qi, ac, at, bon, bC, ei, iu); #c1(WRNIP1) c2(NP_064520) c3(ATPase WRNIP1 isoform 1) c4(202849, 202850) c5(202851, 202852, 202853, 202854, 202855, 202856, 202857, 202858, 202859, 202860, 202861, 202862, 202863) c6( ) c7(MR); #c1(WRNIP1) c2(NP_569079) c3(ATPase WRNIP1 isoform 2) c4(233651, 233652) c5(233653, 233654, 233655, 233656, 233657, 233658, 233659, 233660, 233661, 233662, 233663) c6( ) c7(MR); #c1(WSB1) c2(NP_056441) c3(WD repeat and SOCS box-containing protein 1 isoform 1) c4(187566) c5(187567, 187568, 187569, 187570, 187571, 187572) c6( ) c7(cm, T, aE, K); #c1(WSB1) c2(NP_599027) c3(WD repeat and SOCS box-containing protein 1 isoform 2) c4(235600) c5(235601, 235602, 235603, 235604, 235605, 235606) c6( ) c7(cm, T, aE, K); #c1(WSB1) c2(XP_011522907) c3(WD repeat and SOCS box-containing protein 1 isoform X1) c4(317394) c5(317395, 317396, 317397, 317398, 317399, 317400) c6( ) c7(cm, T, aE, K); #c1(WSCD1) c2(NP_056068) c3(WSC domain-containing protein 1) c4(185495) c5(185496, 185497, 185498, 185499, 185500, 185501, 185502, 185503, 185504, 185505) c6( ) c7(aq); #c1(WSCD2) c2(NP_001291376) c3(WSC domain-containing protein 2) c4(115798) c5(115799, 115800, 115801, 115802, 115803, 115804, 115805, 115806, 115807, 115808) c6( ) c7(bk, aQo, cz); #c1(WSCD2) c2(XP_011537323) c3(WSC domain-containing protein 2 isoform X1) c4(335023) c5(335024, 335025, 335026, 335027, 335028, 335029, 335030, 335031, 335032, 335033) c6( ) c7(bK, aQo, cz); #c1(WT1) c2(NP_000369) c3(Wilms tumor protein isoform A) c4(8199) c5(8200, 8201, 8202, 8203, 8204, 8205, 8206, 8207, 8208, 8209, 8210) c6( ) c7(T, os, aC, aNA, hV, bB, ci, aY, ip, x, aE, PI, K, aGb, aW, lh, hl, ayY, g, eF, bw, bfw, cUV, eM, nB, iA, iT, oy, tV, azg, k, m, Xu, bvC, iu, n, ko, fr, nA, br, cUY, h, UD, aqK, hn, Gc, dx, Nr, cUW, yi, gi, aq, po, Pp, kp, cM, bP, Vy, wk, hL, B, AS, eR, cz, v, Cb, q, er, eA, aeZ, Zp, cUZ, hW, aT, KU, ae, af, CUT, jf, iF, s, vT, iU, Y, Lf, r, gm, Lc, en, hf, bu, t, f, iK, bN, ck, sk, hc, dy, bt, hS, mO, jh, bs, adW, cUX, oZ, ah, hF, ad, Jm, dP, av, hd, fX, ib, py, vJ, vw, cVa, cUU, fb, iL, d, BN, ff, aeq, eH, Ej, iR, aEm, bz, Nq, es, arY, bE, UB, akC, oQ, X); #c1(WT1) c2(NP_001185480) c3(Wilms tumor protein isoform E) c4(75726, 75727) c5(75728, 75729, 75730, 75731, 75732, 75733, 75734, 75735, 75736, 75737, 75738, 75739) c6( ) c7(T, os, aC, aNA, hV, bB, ci, aY, ip, x, aE, PI, K, aGb, aW, lh, hl, ayY, g, eF, bw, bfw, cUV, eM, nB, iA, lT, oy, tV, azg, k, m, Xu, bvC, iu, n, ko, fr, nA, br, cUY, h, UD, aqK, hn, Gc, dx, Nr, cUW, yi, gi, aq, po, Pp, kp, cM, bP, Vy, wk, hL, B, AS, eR, cz, v, Cb, q, er, eA, aeZ, Zp, cUZ, hW, aT, KU, ae, af, cUT, jf, iF, s, vT, iU, Y, Lf, r, gm, Lc, en, hf, bu, t, f, iK, bN, ck, sk, hc, dy, bt, hS, mO, jh, bs, adW, cUX, oZ, ah, hF, ad, Jm, dP, av, hd, fX, ib, py, vJ, vw, cVa, cUU, fb, iL, d, BN, ff, aeq, eH, Ej, iR, aEm, bz, Nq, es, arY, bE, UB, akC, ol, X); #c1(WT1) c2(NP_001185481) c3(Wilms tumor protein isoform F) c4(75740) c5(75741, 75742, 75743, 75744, 75745, 75746) c6( ) c7(T, os, aC, aNA, hV, bB, ci, aY, ip, x, aE, PI, K, aGb, aW, lh, hl, ayY, g, eF, bw, bfw, cUV, eM, nB, iA, iT, oy, tV, azg, k, m, Xu, bvC, iu, n, ko, fr, nA, br, cUY, h, UD, aqK, hn, Gc, dx, Nr, cUW, yi, gi, aq, po, Pp, kp, cM, bP, Vy, wk, hL, B, AS, eR, cz, v, Cb, q, er, eA, aeZ, Zp, cUZ, hW, aT, KU, ae, af, cUT, jf, iF, s, vT, iU, Y, Lf, r, gm, Lc, en, hf, bu, t, f, iK, bN, ck, sk, hc, dy, bt, hS, mO, jh, bs, adW, cUX, oZ, ah, hF, ad, Jm, dP, av, hd, fX, ib, py, vJ, vw, cVa, cUU, fb, iL, d, BN, ff, aeq, eH, Ej, iR, aEm, bz, Nq, es, arY, bE, UB, akC, oQ, X); #c1(WT1) c2(NP_077742) c3(Wilms tumor protein isoform B) c4(215356) c5(215357, 215358, 215359, 215360, 215361, 215362) c6( ) c7(T, os, aC, aNA, hV, bB, ci, aY, ip, x, aE, PI, K, aGb, aW, Ih, hl, ayY, g, eF, bw, bfw, cUV, eM, nB, iA, iT, oy, tV, azg, k, m, Xu, bvC, iu, n, ko, fr, nA, br, cUY, h, UD, aqK, hn, Gc, dx, Nr, cUW, yi, gi, aq, po, Pp, kp, cM, bP, Vy, wk, hL, B, AS, eR, cz, v, Cb, q, er, eA, aeZ, Zp, cUZ, hW, aT, KU, ae, af, cUT, jf, iF, s, vT, iU, Y, Lf, r, gm, Lc, en, hf, bu, t, f, iK, bN, ck, sk, hc, dy, bt, hS, mO, jh, bs, adW, cUX, oZ, ah, hF, ad, Jm, dP, av, hd, fX, ib, py, vJ, vw, cVa, cUU, fb, iL, d, BN, ff, aeq, eH, Ej, iR, aEm, bz, Nq, es, arY, bE, UB, akC, oQ, X); #c1(WT1) c2(NP_077744) c3(Wilms tumor protein isoform D) c4(215363, 215364, 215365) c5(215366, 215367, 215368, 215369, 215370, 215371, 215372, 215373, 215374, 215375, 215376, 215377, 215378) c6( ) c7(T, os, aC, aNA, hV, bB, ci, aY, ip, x, aE, Pl, K, aGb, aW, lh, hl, ayY, g, eF, bw, bfw, cUV, eM, nB, iA, iT, oy, tV, azg, k, m, Xu, bvC, iu, n, ko, fr, nA, br, cUY, h, UD, aqK, hn, Gc, dx, Nr, cUW, yi, gi, aq, po, Pp, kp, cM, bP, Vy, wk, hL, B, AS, eR, cz, v, Cb, q, er, eA, aeZ, Zp, cUZ, hW, aT, KU, ae, af, CUT, jf, iF, s, vT, iU, Y, Lf, r, gm, Lc, en, hf, bu, t, f, iK, bN, ck, sk, hc, dy, bt, hS, mO, jh, bs, adW, cUX, oZ, ah, hF, ad, Jm, dP, av, hd, fX, ib, py, vJ, vw, cVa, cUU, fb, iL, d, BN, ff, aeq, eH, Ej, iR, aEm, bz, Nq, es, arY, bE, UB, akC, oQ, X); #c1(WTAP) c2(NP_001257460) c3(pre-mRNA-splicing regulator WTAP isoform 1) c4(95469, 95470) c5(95471, 95472, 95473, 95474, 95475, 95476) c6( ) c7(T, jz, d, F, Pp, jE); #c1(WTAP) c2(NP_690597) c3(pre-mRNA-splicing regulator WTAP isoform 2) c4( ) c5(244890, 244891, 244892, 244893, 244894, 244895) c6( ) c7(T, jz, d, F, Pp, jE); #c1(WTIP) c2(NP_001073905) c3(Wilms tumor protein (-interacting protein) c4(36426) c5(36427, 36428, 36429, 36430, 36431, 36432) c6( ) c7(Pp, er); #c1(WWC1) c2(NP_001155133) c3(protein KIBRA isoform 1) c4(61464) c5(61465, 61466, 61467, 61468, 61469, 61470) c6( ) c7(T, FW, ot, sO, aW, LX, Ei, aT, aA, gl, bS, aF, ct, Z, cm, cM, af); #c1(WWC1) c2(NP_056053) c3(protein KIBRA isoform 3) c4(185398) c5(185399, 185400, 185401, 185402, 185403, 185404) c6( ) c7(T, FW, ot, sO, aW, LX, Ei, aT, aA, gl, bS, aF, ct, Z, cm, cM, af); #c1(WWC2) c2(NP_079225) c3(protein WWC2) c4(217520, 217521, 217522, 217523) c5(217524, 217525, 217526, 217527, 217528, 217529, 217530, 217531, 217532, 217533, 217534, 217535, 217536, 217537, 217538, 217539, 217540, 217541, 217542, 217543, 217544, 217545, 217546, 217547) c6( ) c7(De); #c1(WWC2) c2(XP_011530571) c3(protein WWC2 isoform X1) c4(326486) c5(326487, 326488, 326489, 326490, 326491, 326492) c6( ) c7(De); #c1(WWOX) c2(NP_001278926) c3(WW domain-containing oxidoreductase isoform 4) c4( ) c5(111016, 111017, 111018) c6( ) c7(T, os, aC, id, iB, Dd, oe, ha, e, ht, fZ, aA, bB, aj, v, j, ci, nA, oZ, qN, hF, AD, fr, q, ad, er, eA, ip, aw, ft, hd, aE, jz, eL, aT, io, h, dL, bK, K, bta, hB, an, ae, af, eL, WW, if, d, Jl, aW, ff, cVb, eH, r, dq, lo, af, PQ, en, hf, gW, t, QK, fq, bz, g, eF, cm, ck, iK, fd, ky, nB, bE, eM, go, bo, it, nw, m, adg, Pr, B); #c1(WWOX) c2(NP_057457) c3(WW domain-containing oxidoreductase isoform 1) c4(190997) c5(190998, 190999, 191000, 191001, 191002, 191003, 191004, 191005, 191006, 191007, 191008, 191009, 191010) c6( ) c7(T, os, aC, id, iB, Dd, oe, ha, e, ht, fZ, aA, bB, aj, v, j, ci, nA, oZ, qN, hF, AO, fr, q, ad, er, eA, ip, aw, ft, hd, aE, jz, eO, aT, io, h, dL, bK, K, bta, hB, an, ae, af, eL, WW, if, d, Jl, aW, ff, cVb, eH, r, dq, lo, aF, PQ, en, hf, gW, t, QK, fq, bz, g, eF, cm, ck, iK, fd, ky, nB, bE, eM, go, bo, it, nw, m, adg, Pr, B); #c1(WWOX) c2(NP_570607) c3(WW domain-containing oxidoreductase isoform 2) c4(234090) c5(234091, 234092, 234093, 234094, 234095, 234096, 234097, 234098, 234099, 234100, 234101, 234102, 234103, 234104, 234105, 234106, 234107, 234108, 234109, 234110, 234111, 234112, 234113, 234114, 234115, 234116, 234117, 234118, 234119, 234120) c6( ) c7(T, os, aC, id, iB, Dd, oe, ha, e, ht, fZ, aA, bB, aj, v, j, ci, nA, oZ, qN, hF, AO, fr, q, ad, er, eA, ip, aw, ft, hd, aE, jz, eL, al, io, h, dL, bK, K, bta, hB, an, ae, af, eL, WW, if, d, Jl, aW, ff, cVb, eH, r, dq, lo, aF, PQ, en, hf, gW, t, QK, fq, bz, g, eF, cm, ck, iK, fd, ky, nB, bE, eM, go, bo, it, nw, m, adg, Pr, B); #c1(WWOX) c2(XP_011521402) c3(WW domain-containing oxidoreductase isoform X1) c4(316216) c5( ) c6( ) c7(T, os, aC, id, iB, Dd, oe, ha, e, ht, fZ, aA, bB, aj, v, j, ci, nA, oZ, qN, hF, AO, fr, q, ad, er, eA, ip, aw, ft, hd, aE, jz, eO, aT, io, h, dL, bK, K, bta, hB, an, ae, af, eL, WW, if, d, Jl, aW, ff, cVb, eH, r, dq, lo, aF, PQ, en, hf, gW, t, QK, fq, bz, g, eF, cm, ck, iK, fd, ky, nB, bE, eM, go, bo, it, nw, m, adg, Pr, B); #c1(WWOX) c2(XP_011521404) c3(WW domain-containing oxidoreductase isoform X3) c4(316217, 316218) c5( ) c6( ) c7(T, os, aC, id, iB, Dd, oe, ha, e, ht, fZ, aA, bB, aj, v, j, ci, nA, oZ, qN, hF, AD, fr, q, ad, er, eA, ip, aw, ft, hd, aE, jz, eL, aT, io, h, dL, bK, K, bta, hB, an, ae, af, eL, WW, if, d, Jl, aW, ff, cVb, eH, r, dq, lo, aF, PQ, en, hf, gW, t, QK, fq, bz, g, eF, cm, ck, iK, fd, ky, nB, bE, eM, go, bo, it, nw, m, adg, Pr, B); #c1(WWOX) c2(XP_011521405) c3(WW domain-containing oxidoreductase isoform X4) c4(316219) c5( ) c6( ) c7(T, os, aC, id, iB, Dd, oe, ha, e, ht, fZ, aA, bB, aj, v, j, ci, nA, oZ, qN, hF, AO, fr, q, ad, er, eA, ip, aw, ft, hd, aE, jz, eO, aT, io, h, dL, bK, K, bta, hB, an, ae, af, eL, WW, if, d, Jl, aW, ff, cVb, eH, r, dq, lo, aF, PQ, en, hf, gW, t, QK, fq, bz, g, eF, cm, ck, iK, fd, ky, nB, bE, eM, go, bo, it, nw, m, adg, Pr, B); #c1(WWOX) c2(XP_011521406) c3(WW domain-containing oxidoreductase isoform X6) c4(316220) c5( ) c6( ) c7(T, os, aC, id, iB, Dd, oe, ha, e, ht, fZ, aA, bB, aj, v, j, ci, nA, oZ, qN, hF, AD, fr, q, ad, er, eA, ip, aw, ft, hd, aE, jz, eO, aT, io, h, dL, bK, K, bta, hB, an, ae, af, eL, WW, if, d, Jl, aW, ff, cVb, eH, r, dq, lo, aF, PQ, en, hf, gW, t, QK, fq, bz, g, eF, cm, ck, iK, fd, ky, nB, bE, eM, go, bo, it, nw, m, adg, Pr, B); #c1(WWP1) c2(XP_005250817) c3(NEDD4-like E3 ubiquitin-protein ligase WWP1 isoform X1) c4(270559, 270560, 270561) c5(270562, 270563, 270564, 270565, 270566, 270567, 270568, 270569, 270570, 270571) c6( ) c7(T, aW, aT, in, ae, ad); #c1(WWP1) c2(XP_005250818) c3(NEDD4-like E3 ubiquitin-protein ligase WWP1 isoform X2) c4(270572) c5(270573, 270574, 270575, 270576, 270577, 270578, 270579, 270580, 270581, 270582, 270583, 270584, 270585) c6( ) c7(T, aW, aT, in, ae, ad); #c1(WWP1) c2(XP_011515104) c3(NEDD4-like E3 ubiquitin-protein ligase WWP1 isoform X3) c4( ) c5(307973, 307974, 307975, 307976, 307977) c6( ) c7(T, aW, aT, in, ae, ad); #c1(WWP2) c2(NP_001257384) c3(NEDD4-like E3 ubiquitin-protein ligase WWP2 isoform WWP2-N) c4( ) c5(95296, 95297, 95298, 95299, 95300, 95301) c6( ) c7(aw, aRo); #c1(WWP2) c2(NP_955456) c3(NEDD4-like E3 ubiquitin-protein ligase WWP2 isoform WWP2-C) c4( ) c5(261766, 261767, 261768, 261769, 261770) c6( ) c7(aw, aRo); #c1(WWP2) c2(XP_011521127) c3(NEDD4-like E3 ubiquitin-protein ligase WWP2 isoform X1) c4(316038, 316039, 316040, 316041, 316042, 316043) c5(316044, 316045, 316046, 316047, 316048, 316049, 316050, 316051, 316052, 316053, 316054, 316055, 316056, 316057, 316058, 316059, 316060, 316061, 316062, 316063, 316064, 316065) c6( ) c7(aw, aRo); #c1(WWP2) c2(XP_011521128) c3(NEDD4-like E3 ubiquitin-protein ligase WWP2 isoform X2) c4( ) c5(316066, 316067, 316068, 316069, 316070, 316071, 316072, 316073, 316074, 316075, 316076) c6( ) c7(aw, aRo); #c1(WWTR1) c2(NP_001161750) c3(WW domain-containing transcription regulator protein 1) c4(65913, 65914, 65915, 65916, 65917) c5(65918, 65919, 65920, 65921, 65922, 65923, 65924, 65925, 65926, 65927, 65928, 65929, 65930, 65931, 65932, 65933, 65934, 65935, 65936, 65937, 65938, 65939, 65940, 65941, 65942, 65943, 65944, 65945, 65946, 65947, 65948, 65949) c6( ) c7(gV, cSS, T, dD, atk, fd, bB, afK, eq, aoM, cd, q, vN, eA, cc, aT, bl, hH, azH, d, hG, aW, Y, cmH, r, bp, dw, t, bz, Qt, dr, bfW, Dy, eM, bE, mx, wB, dy, m, ym, cba); #c1(XAB2) c2(NP_064581) c3(pre-mRNA-splicing factor SYF1) c4(203084, 203085) c5(203086, 203087, 203088, 203089, 203090, 203091, 203092, 203093, 203094, 203095, 203096, 203097) c6( ) c7(bv, T, aXH, j, V, kS); #c1(XAF1) c2(NP_059993) c3(XIAP-associated factor 1 isoform 1) c4(193547, 193548, 193549) c5(193550, 193551, 193552, 193553, 193554, 193555, 193556, 193557, 193558, 193559, 193560, 193561, 193562, 193563, 193564, 193565, 193566, 193567) c6( ) c7(T, aA, bB, n, fd, fr, ci, qN, V, ad, hX, ds, dL, K, ae, eL, uQ, d, Y, r, aq, aF, g, iR, cm, QS, es, bE, AH, X, gX, B); #c1(XAF1) c2(NP_954590) c3(XIAP-associated factor 1 isoform 2) c4(261072) c5(261073, 261074, 261075, 261076, 261077, 261078) c6( ) c7(T, aA, bB, n, fd, fr, ci, qN, V, ad, hX, ds, dL, K, ae, eL, uQ, d, Y, r, aq, aF, g, iR, cm, QS, es, bE, AH, X, gX, B); #c1(XAF1) c2(XP_005256765) c3(XIAP-associated factor 1 isoform X7) c4(274480) c5(274481, 274482, 274483, 274484, 274485, 274486) c6( ) c7(T, aA, bB, n, fd, fr, ci, qN, V, ad, hX, ds, dL, K, ae, eL, uQ, d, Y, r, aq, aF, g, iR, cm, QS, es, bE, AH, X, gX, B); #c1(XAGE1B) c2(NP_001091065) c3(X antigen family member 1 isoform d) c4(38413) c5(38414, 38415, 38416, 38417, 38418, 38419, 38420, 38421, 38422, 38423, 38424, 38425, 38426, 38427, 38428, 38429, 38430, 38431, 38432, 38433, 38434, 38435, 38436, 38437, 38438, 38439, 38440, 38441, 38442, 38443, 38444, 38445, 38446, 38447) c6( ) c7(av, T, Y, qK, Xu, bu, ae, iu, ad); #c1(XAGE1E) c2(NP_001091073) c3(X antigen family member 1 isoform a) c4(38459, 38460) c5(38461, 38462, 38463, 38464, 38465, 38466, 38467, 38468, 38469, 38470, 38471, 38472, 38473, 38474, 38475, 38476, 38477, 38478, 38479, 38480, 38481, 38482, 38483, 38484, 38485, 38486, 38487, 38488, 38489, 38490, 38491, 38492, 38493, 38494, 38495, 38496, 38497, 38498, 38499, 38500, 38501, 38502, 38503, 38504, 38505, 38506) c6( ) c7(T, Y, Xu, bu, ae, iu, ad); #c1(XBP1) c2(NP_001073007) c3(X-box-binding protein 1 isoform XBP1(S) c4(35580) c5(35581, 35582, 35583, 35584, 35585, 35586, 35587, 35588, 35589) c6(35590, 35591) c7(bL, lD, T, dD, fM, ea, iW, eX, bO, fd, jh, mS, NJ, v, qj, ct, ci, PJ, EA, eq, eA, zw, pe, mW, aT, Ne, cy, jy, dL, bK, K, eV, ji, oi, dF, ly, je, aai, aW, Y, Of, fc, fe, Og, g, aom, Z, t, iV, lx, yl, iM, c, eM, aiM, cj, vl, at, uJ, wz, m, cP, ei, y, bt, B, pC); #c1(XBP1) c2(NP_005071) c3(X-box-binding protein 1 isoform XBP1(U)) c4(152608, 152609, 152610) c5(152611, 152612, 152613, 152614, 152615, 152616, 152617, 152618, 152619, 152620, 152621, 152622, 152623, 152624, 152625, 152626, 152627, 152628, 152629) c6( ) c7(bL, lD, T, dD, fM, ea, iW, eX, bO, fd, jh, mS, NJ, v, qj, ct, ci, PJ, EA, eq, eA, zw, pe, mW, aT, Ne, cy, jy, dL, bK, K, eV, ji, oi, dF, ly, je, aai, aW, Y, Of, fc, fe, Og, g, aom, Z, t, iV, lx, yl, iM, c, eM, aiM, cj, vl, at, uJ, wz, m, cP, ei, y, bt, B, pC); #c1(XCL1) c2(XP_011508167) c3(lymphotactin isoform X1) c4(298701) c5(298702, 298703, 298704, 298705, 298706, 298707) c6( ) c7(bM, aW, aCJ, dh, CM, aT, cj, aA, cz, aF, ey, m, ci, pb, h, y, ME, B, aj, bA); #c1(XCL2) c2(NP_003166) c3(cytokine SCM-1 beta precursor) c4(135846) c5(135847, 135848, 135849, 135850, 135851, 135852) c6( ) c7(B, m); #c1(XCR1) c2(NP_001019815) c3(chemokine XC receptor 1) c4(27901, 27902) c5(27903, 27904, 27905, 27906, 27907, 27908, 27909, 27910, 27911, 27912, 27913, 27914, 27915, 27916, 27917, 27918) c6( ) c7(h, hH, hp, m); #c1(XDH) c2(NP_000370) c3(xanthine dehydrogenase/oxidase) c4(8211) c5(8212, 8213, 8214, 8215, 8216, 8217) c6( ) c7(bM, T, dD, wi, fR, ea, e, ht, al, fN, aA, cR, cz, bB, Xn, eq, ci, C, sl, zP, q, cd, ip, fU, nH, iu, cc, dY, HC, aT, lG, cy, DU, K, wC, zQ, aW, ff, r, fe, aF, en, t, bz, dr, bN, ar, dB, cLU, bE, gZ, E, at, wx, dy, k, Hb, m, B, rk); #c1(XG) c2(NP_001135391) c3(glycoprotein Xg isoform 2 precursor) c4(52853, 52854) c5(52855, 52856, 52857, 52858, 52859, 52860) c6( ) c7(buR, xs, KW, Ri, hQ, uY, btu, Km, cVc, yn, cVd); #c1(XG) c2(NP_001135392) c3(glycoprotein Xg isoform 3 precursor) c4(52861) c5(52862, 52863, 52864, 52865, 52866, 52867) c6( ) c7(buR, xs, KW, Ri, hl, uY, btu, Km, cVc, yn, cVd); #c1(XG) c2(NP_780778) c3(glycoprotein Xg isoform 1 precursor) c4(251560) c5(251561, 251562, 251563, 251564, 251565, 251566, 251567, 251568, 251569, 251570, 251571) c6( ) c7(buR, xs, KW, Ri, hQ, uY, btu, Km, cVc, yn, cVd); #c1(XG) c2(XP_011543878) c3(glycoprotein Xg isoform X7) c4( ) c5(343050) c6( ) c7(buR, xs, KW, Ri, hQ, uY, btu, Km, cVc, yn, cVd); #c1(XIAP) c2(XP_011529631) c3(E3 ubiquitin-protein ligase XIAP isoform X1) c4(325158, 325159, 325160, 325161, 325162, 325163) c5(325164, 325165, 325166, 325167, 325168, 325169, 325170, 325171, 325172, 325173, 325174, 325175, 325176, 325177, 325178, 325179, 325180, 325181, 325182, 325183, 325184, 325185, 325186, 325187, 325188, 325189, 325190, 325191, 325192, 325193, 325194, 325195, 325196, 325197, 325198, 325199, 325200, 325201, 325202, 325203, 325204, 325205, 325206, 325207, 325208, 325209, 325210, 325211, 325212, 325213, 325214, 325215, 325216, 325217, 325218, 325219, 325220, 325221, 325222, 325223, 325224, 325225, 325226, 325227, 325228, 325229, 325230, 325231, 325232, 325233, 325234, 325235, 325236, 325237, 325238, 325239, 325240, 325241, 325242, 325243, 325244, 325245, 325246, 325247, 325248, 325249, 325250, 325251, 325252, 325253, 325254, 325255, 325256, 325257, 325258, 325259, 325260, 325261, 325262, 325263, 325264, 325265, 325266, 325267, 325268, 325269, 325270, 325271, 325272, 325273, 325274, 325275, 325276, 325277, 325278, 325279, 325280, 325281, 325282) c6(325283, 325284) c7(gh, T, bM, aC, fR, if, eb, bB, fd, ci, bv, aE, Bc, K, eL, akV, YY, aW, ih, g, cm, bw, eM, nB, iA, cj, m, QS, cZ, wu, ht, aj, fr, qN, PJ, zP, cd, ft, jz, hw, kH, eV, dL, dd, MD, fe, kp, dw, bQL, Wt, B, fM, dA, eX, aA, hz, hy, V, q, J, eA, uT, aT, vH, cy, ae, gP, iU, nO, oV, Y, r, aZg, t, iK, ck, nM, iM, c, bt, Ze, zA, aDj, LS, kh, jh, dG, pz, hF, ad, afb, av, ib, NY, Dq, wm, uQ, d, BN, je, aF, bz, LB, es, bE, X, y, ne); #c1(XIRP1) c2(NP_001185550) c3(xin actin-binding repeat-containing protein 1 isoform 2) c4(75825) c5(75826, 75827, 75828, 75829, 75830, 75831) c6( ) c7(aj); #c1(XIRP1) c2(NP_919269) c3(xin actin-binding repeat-containing protein 1 isoform) c4(258368, 258369) c5(258370, 258371, 258372, 258373, 258374, 258375, 258376, 258377, 258378, 258379, 258380, 258381) c6( ) c7(aj); #c1(XIRP2) c2(NP_001073278) c3(xin actin-binding repeat-containing protein 2 isoform 2) c4(35708) c5(35709, 35710, 35711, 35712, 35713, 35714, 35715, 35716, 35717, 35718, 35719) c6( ) c7(aWm, aw, bN); #c1(XIRP2) c2(NP_001186072) c3(xin actin-binding repeat-containing protein 2 isoform 3) c4(77020, 77021) c5(77022, 77023, 77024, 77025, 77026, 77027, 77028, 77029, 77030, 77031) c6( ) c7(aWm, aw, bN); #c1(XIRP2) c2(NP_001186073) c3(xin actin-binding repeat-containing protein 2 isoform 4) c4(77032, 77033) c5(77034, 77035, 77036, 77037, 77038, 77039, 77040, 77041, 77042, 77043, 77044) c6( ) c7(aWm, aw, bN); #c1(XIRP2) c2(NP_001186074) c3(xin actin-binding repeat-containing protein 2 isoform 5) c4( ) c5(77045, 77046, 77047, 77048, 77049) c6( ) c7(aWm, aw, bN); #c1(XIRP2) c2(NP_689594) c3(xin actin-binding repeat-containing protein 2 isoform 1) c4(243123) c5(243124, 243125, 243126, 243127, 243128, 243129, 243130, 243131, 243132, 243133, 243134) c6( ) c7(aWm, aw, bN); #c1(XK) c2(NP_066569) c3(membrane transport protein XK precursor) c4(207434) c5(207435, 207436, 207437, 207438, 207439, 207440) c6( ) c7(T, mr, aA, oY, fr, bX, bgl, Ak, zU, K, he, ZM, af, iw, aF, as, cm, xw, iA, U, iu, L); #c1(XKR4) c2(NP_443130) c3(XK-related protein 4) c4(229571) c5(229572, 229573, 229574, 229575, 229576, 229577, 229578, 229579, 229580, 229581) c6( ) c7(aw, lB, oe, cd); #c1(XKR6) c2(NP_775954) c3(XK-related protein 6) c4(250778, 250779) c5(250780, 250781, 250782, 250783, 250784, 250785, 250786, 250787, 250788, 250789, 250790, 250791) c6( ) c7(c0, eZ); #c1(XKR9) c2(NP_001274188) c3(XK-related protein 9 isoform 2) c4(106138) c5(106139, 106140, 106141, 106142, 106143, 106144, 106145, 106146, 106147, 106148) c6( ) c7(ad); #c1(XKR9) c2(XP_011515829) c3(XK-related protein 9 isoform X4) c4(308787) c5(308788, 308789, 308790, 308791, 308792, 308793) c6( ) c7(ad); #c1(XPA) c2(NP_000371) c3(DNA repair protein complementing XP-A cells) c4(8218) c5(8219, 8220, 8221, 8222, 8223, 8224) c6( ) c7(gV, T, GI, gU, aC, yk, ht, jh, aA, n, hy, oZ, Gn, ci, fr, Do, V, q, ad, eW, ke, av, iu, hw, aE, aXH, avA, afQ, rC, yg, aT, rA, dL, lQ, ae, Qe, aW, Y, ff, eH, CT, r, af, La, en, bu, cl, t, g, bz, eF, op, cm, iK, es, sS, Zi, eM, iT, hk, pP, hL, m, bt, Vj, B); #c1(XPC) c2(NP_004619) c3(DNA repair protein complementing XP-C cells) c4(148230, 148231, 148232) c5(148233, 148234, 148235, 148236, 148237, 148238, 148239, 148240, 148241, 148242, 148243, 148244, 148245, 148246, 148247, 148248) c6( ) c7(T, aC, pf, if, ci, ip, afr, aE, u, Qk, ja, K, hu, eL, aW, sP, CT, La, g, eF, zE, fT, eM, cj, m, gU, ht, n, fr, h, eV, dL, hB, FZ, ks, aq, dB, hk, Vy, hL, at, nL, NB, it, B, yk, jx, eX, aA, hy, Do, V, q, eA, yg, aT, vH, ae, s, ND, aBq, Y, r, en, cl, t, iK, bN, sS, rR, Dy, rA, bt, gV, GI, id, Gn, ad, hX, av, l, io, rL, WW, d, hG, ff, aF, bz, dr, es, Zi, iu); #c1(XPC) c2(XP_011532394) c3(DNA repair protein complementing XP-C cells isoform X1) c4(328551, 328552) c5( ) c6( ) c7(T, aC, pf, if, ci, ip, afr, aE, u, Qk, ja, K, hu, eL, aW, sP, CT, La, g, eF, zE, fT, eM, cj, m, gU, ht, n, fr, h, eV, dL, hB, FZ, ks, aq, dB, hk, Vy, hL, at, nL, NB, it, B, yk, jx, eX, aA, hy, Do, V, q, eA, yg, aT, vH, ae, s, ND, aBq, Y, r, en, cl, t, iK, bN, sS, rR, Dy, rA, bt, gV, GI, id, Gn, ad, hX, av, l, io, rL, WW, d, hG, ff, aF, bz, dr, es, Zi, iu); #c1(XPC) c2(XP_011532395) c3(DNA repair protein complementing XP-C cells isoform X2) c4(328553, 328554) c5(328555, 328556, 328557, 328558, 328559, 328560, 328561, 328562, 328563, 328564, 328565, 328566, 328567, 328568, 328569, 328570, 328571, 328572, 328573, 328574, 328575, 328576, 328577, 328578, 328579, 328580) c6( ) c7(T, aC, pf, if, ci, ip, afr, aE, u, Ok, ja, K, hu, eL, aW, sP, CT, La, g, eF, zE, fT, eM, cj, m, gU, ht, n, fr, h, eV, dL, hB, FZ, ks, aq, dB, hk, Vy, hl, at, nL, NB, it, B, yk, jx, eX, aA, hy, Do, V, q, eA, yg, aT, vH, ae, s, ND, aBq, Y, r, en, cl, t, iK, bN, sS, rR, Dy, rA, bt, gV, GI, id, Gn, ad, hX, av, l, io, rL, WW, d, hG, ff, aF, bz, dr, es, Zi, iu); #c1(XPNPEP1) c2(NP_001161076) c3(xaa-Pro aminopeptidase 1 isoform 2) c4(65265) c5(65266, 65267, 65268, 65269, 65270, 65271) c6( ) c7(bF, Fq, f, cm, af, L); #c1(XPNPEP1) c2(NP_065116) c3(xaa-Pro aminopeptidase 1 isoform 1) c4(204003, 204004, 204005) c5(204006, 204007, 204008, 204009, 204010, 204011, 204012, 204013, 204014, 204015, 204016, 204017, 204018, 204019, 204020, 204021, 204022) c6( ) c7(bF, Fq, f, cm, af, L); #c1(XPNPEP1) c2(XP_011538435) c3(xaa-Pro aminopeptidase 1 isoform X5) c4(336229) c5( ) c6( ) c7(bF, Fq, f, cm, af, L); #c1(XPNPEP1) c2(XP_011538436) c3(xaa-Pro aminopeptidase 1 isoform X6) c4( ) c5(336230, 336231, 336232, 336233, 336234, 336235) c6( ) c7(bF, Fq, f, cm, af, L); #c1(XPNPEP2) c2(NP_003390) c3(xaa-Pro aminopeptidase 2 precursor) c4(138134, 138135) c5(138136, 138137, 138138, 138139, 138140, 138141, 138142, 138143, 138144, 138145, 138146, 138147) c6( ) c7(cVf, cVe); #c1(XPNPEP3) c2(NP_071381) c3(probable Xaa-Pro aminopeptidase 3 isoform 1) c4(211285, 211286, 211287, 211288) c5(211289, 211290, 211291, 211292, 211293, 211294, 211295, 211296, 211297, 211298, 211299, 211300, 211301, 211302, 211303, 211304, 211305, 211306, 211307, 211308, 211309, 211310, 211311, 211312) c6( ) c7(cVg, dy, vm); #c1(XP01) c2(XP_011531399) c3(exportin-1 isoform X1) c4(327396, 327397, 327398, 327399, 327400) c5(327401, 327402, 327403, 327404, 327405, 327406, 327407, 327408, 327409, 327410, 327411, 327412, 327413, 327414, 327415, 327416, 327417, 327418, 327419) c6( ) c7(T, fd, oZ, fr, ci, eA, ad, hX, qC, x, aE, dV, Ne, h, K, rz, ae, je, Y, aq, eF, t, iR, bN, dR, ck, es, bF, iM, kP, eM, iA, cj, m, k, bD, bt, B); #c1(XP01) c2(XP_011531400) c3(exportin-1 isoform X3) c4(327420, 327421, 327422) c5(327423, 327424, 327425, 327426, 327427, 327428, 327429, 327430, 327431, 327432, 327433, 327434, 327435, 327436, 327437, 327438, 327439, 327440, 327441, 327442, 327443, 327444, 327445, 327446, 327447, 327448, 327449, 327450, 327451, 327452, 327453, 327454, 327455, 327456, 327457, 327458, 327459) c6( ) c7(T, fd, oZ, fr, ci, eA, ad, hX, qC, x, aE, dV, Ne, h, K, rz, ae, je, Y, aq, eF, t, iR, bN, dR, ck, es, bF, iM, kP, eM, iA, cj, m, k, bD, bt, B); #c1(XP04) c2(NP_071904) c3(exportin-4) c4(212192, 212193) c5(212194, 212195, 212196, 212197, 212198, 212199, 212200, 212201, 212202, 212203, 212204, 212205, 212206, 212207, 212208, 212209, 212210, 212211) c6( ) c7(pN, al, aq, bK, jE, iR, cm, B, hX); #c1(XP04) c2(XP_011533499) c3(exportin-4 isoform X1) c4(329589) c5( ) c6( ) c7(pN, al, aq, bK, jE, iR, cm, B, hX); #c1(XP05) c2(NP_065801) c3(exportin-5) c4(205561, 205562, 205563, 205564, 205565) c5(205566, 205567, 205568, 205569, 205570, 205571, 205572, 205573, 205574, 205575, 205576, 205577, 205578, 205579, 205580, 205581, 205582, 205583, 205584, 205585, 205586, 205587, 205588, 205589, 205590, 205591, 205592, 205593, 205594, 205595) c6( ) c7(XX, es, T, id, BN, vJ, iI, hy, fr, ci, ne); #c1(XP06) c2(NP_001257869) c3(exportin-6 isoform 1) c4( ) c5(95690, 95691, 95692, 95693, 95694) c6( ) c7(IB); #c1(XP06) c2(NP_055986) c3(exportin-6 isoform 2) c4(184911, 184912) c5(184913, 184914, 184915, 184916, 184917, 184918, 184919) c6( ) c7(IB); #c1(XP07) c2(NP_055839) c3(exportin-7) c4(183798, 183799, 183800) c5(183801, 183802, 183803, 183804, 183805, 183806, 183807, 183808, 183809, 183810, 183811, 183812, 183813, 183814, 183815, 183816, 183817, 183818) c6( ) c7(aw); #c1(XPR1) c2(NP_001129141) c3(xenotropic and polytropic retrovirus receptor 1 isoform 2) c4(50888) c5(50889, 50890, 50891, 50892, 50893, 50894) c6( ) c7(bL, T, dD, ea, jx, bB, v, eq, fr, wy, q, ad, hX, jF, l, hd, aE, yw, u, dn, aT, ji, K, jj, ae, pi, aW, eH, hl, LZ, r, fc, aq, g, hJ, eF, cm, bz, iR, bbT, jq, pv, ej, W, eM, bE, fD, ei, y, kS, B); #c1(XPR1) c2(NP_004727) c3(xenotropic and polytropic retrovirus receptor 1 isoform 1) c4(149116) c5(149117, 149118, 149119, 149120, 149121, 149122) c6( ) c7(bL, T, dD, ea, jx, bB, v, eq, fr, wy, q, ad, hX, jF, l, hd, aE, yw, u, dn, aT, ji, K, jj, ae, pi, aW, eH, hl, LZ, r, fc, aq, g, hJ, eF, cm, bz, iR, bbT, jq, pv, ej, W, eM, bE, fD, ei, y, kS, B); #c1(XRCC1) c2(NP_006288) c3(DNA repair protein XRCC1) c4(163685, 163686, 163687, 163688, 163689) c5(163690, 163691, 163692, 163693, 163694, 163695, 163696, 163697, 163698, 163699, 163700, 163701, 163702, 163703, 163704, 163705, 163706, 163707, 163708, 163709, 163710, 163711, 163712, 163713, 163714, 163715, 163716, 163717) c6( ) c7(atT, T, aC, pf, atj, if, al, aHx, bB, qj, ci, aY, aR, ip, aE, u, Qk, K, hu, pk, eL, Zm, cp, cOV, z, dH, axz, aW, CT, adp, nY, kt, LZ, NR, g, zE, uC, Qx, ou, fT, y, eM, nB, iA, iT, go, vA, cj, k, m, cE, iu, Vj, gU, Uc, QO, WA, sw, aWG, ht, NT, aw, n, aj, fr, qN, rP, b, ft, hw, qg, i, gd, lG, eV, dL, Gw, hB, ask, Xh, ny, FZ, AQ, ge, aq, cM, Zj, op, bbz, Sc, W, bP, dB, hk, Qu, PI, hL, gw, at, NB, Pr, B, it, bL, fM, eR, yk, eX, aA, hy, v, j, Do, Zk, V, AO, q, eA, eJ, RI, afQ, cVh, aT, vH, Tj, ae, af, eu, qw, avV, Sh, s, vT, nO, iU, Y, yL, r, lo, en, bu, cl, t, Vi, iK, pb, ck, Qi, sS, nM, ac, Dy, kP, alt, c, pZ, iQ, gX, fD, bt, rA, VB, ao, hS, gV, GI, hl, id, nZ, Zl, aD, rQ, iW, jh, vr, Gn, jE, asl, hF, ad, kL, hX, dP, av, Tg, l, hd, ib, Rq, Db, py, NS, io, rx, hD, Yi, WW, BN, bug, hG, ff, eZ, Ej, aF, iV, gW, bz, es, Zi, bE, Lb, bC, Gu, adg, hE, ne, rk, pC); #c1(XRCC2) c2(NP_005422) c3(DNA repair protein XRCC2) c4(155995) c5(155996, 155997, 155998, 155999, 156000, 156001) c6( ) c7(GI, T, os, aC, yk, iW, ht, aA, hy, oZ, Do, V, q, eA, ip, aE, NS, Qk, bkP, aT, Gw, dL, eL, qw, aW, Y, ff, r, Am, NR, en, g, zE, t, ck, sS, eM, nB, dB, vA, cj, m, Gu); #c1(XRCC3) c2(XP_005268103) c3(DNA repair protein XRCC3 isoform X1) c4(281521, 281522, 281523, 281524, 281525, 281526) c5(281527, 281528, 281529, 281530, 281531, 281532, 281533, 281534, 281535, 281536, 281537, 281538, 281539, 281540, 281541, 281542, 281543, 281544, 281545, 281546, 281547, 281548, 281549, 281550, 281551, 281552, 281553, 281554, 281555, 281556, 281557, 281558, 281559, 281560, 281561, 281562, 281563, 281564, 281565, 281566, 281567, 281568, 281569, 281570, 281571, 281572, 281573, 281574, 281575, 281576, 281577, 281578) c6( ) c7(atT, T, aC, pf, if, fd, ci, ip, aE, Qk, hu, eL, zQ, aW, CT, nY, g, as, bw, eM, nB, vA, m, cE, aw, ht, NT, fr, qN, zP, aJI, hw, Nn, eV, Gw, dL, Ck, ny, cM, dB, hk, hL, at, WL, B, yk, eX, aA, hy, j, bX, Do, Zk, V, q, eA, eJ, bIN, afQ, aT, ae, qw, s, Y, r, en, t, iK, ck, sS, Dy, bt, gV, GI, aD, iW, oZ, Gn, asl, ad, av, hd, ib, Rq, Db, yt, lQ, WW, d, BN, aOb, ff, eZ, aF, bz, es, Gu); #c1(XRCC4) c2(NP_072044) c3(DNA repair protein XRCC4 isoform 1) c4(212439) c5(212440, 212441, 212442, 212443, 212444, 212445, 212446, 212447, 212448, 212449) c6( ) c7(bL, T, os, dA, iW, jh, aA, fd, oZ, fr, V, iJ, q, ad, eA, ip, dP, aHu, Db, Qk, aT, dL, eL, aW, ff, hl, r, aq, aF, en, g, iR, cm, bz, avx, op, pb, bJ, ck, eM, nB, dB, vA, cj, at, m, B); #c1 (XRCC4) c2(XP_011541931) c3(DNA repair protein XRCC4 isoform X4) c4( ) c5(340449, 340450, 340451, 340452, 340453) c6( ) c7(bL, T, os, dA, iW, jh, aA, fd, oZ, fr, V, iJ, q, ad, eA, ip, dP, aHu, Db, Qk, aT, dL, eL, aW, ff, hl, r, aq, aF, en, g, iR, cm, bz, avx, op, pb, bJ, ck, eM, nB, dB, vA, cj, at, m, B); #c1(XRCC5) c2(NP_066964) c3(X-ray repair cross-complementing protein 5) c4(207847) c5(207848, 207849, 207850, 207851, 207852, 207853, 207854, 207855, 207856, 207857) c6( ) c7(T, os, id, al, aA, bB, n, hy, oZ, j, ci, fd, V, mC, q, eA, hX, ip, iu, eW, aT, Ou, dL, hS, eL, CB, B, BN, s, iU, aW, Y, ff, eL, r, aq, alb, aF, en, g, iR, pm, iK, bz, eM, nB, dB, bE, iT, cj, at, fL, qF, m, hh, bC, it, ne, Ro); #c1(XRCC6) c2(NP_001275905) c3(X-ray repair cross-complementing protein 6 isoform 1) c4(107318, 107319, 107320) c5(107321, 107322, 107323, 107324, 107325, 107326, 107327, 107328, 107329, 107330, 107331, 107332, 107333, 107334, 107335, 107336, 107337, 107338, 107339, 107340) c6( ) c7(T, os, id, dA, if, ht, eX, aA, bB, n, hy, oZ, fr, ci, qN, fd, V, mC, q, ad, eA, eW, ib, aT, cy, eV, dL, hu, ae, eL, pl, d, s, iU, aW, Y, ff, eZ, r, alb, aF, en, g, cm, iK, bz, Ye, rR, eM, iT, dB, bE, cj, fL, m, WL, Vj, B); #c1(XRCC6) c2(NP_001275906) c3(X-ray repair cross-complementing protein 6 isoform 2) c4(107341) c5(107342, 107343, 107344, 107345, 107346, 107347) c6( ) c7(T, os, id, dA, if, ht, eX, aA, bB, n, hy, oZ, fr, ci, qN, fd, V, mC, q, ad, eA, eW, ib, aT, cy, eV, dL, hu, ae, eL, pl, d, s, iU, aW, Y, ff, eZ, r, alb, af, en, g, cm, iK, bz, Ye, rR, eM, iT, dB, bE, cj, fL, m, WL, Vj, B); #c1(XRCC6) c2(NP_001275907) c3(X-ray repair cross-complementing protein 6 isoform 3) c4(107348) c5(107349, 107350, 107351, 107352, 107353, 107354, 107355, 107356, 107357, 107358, 107359, 107360) c6( ) c7(T, os, id, dA, if, ht, eX, aA, bB, n, hy, oZ, fr, ci, qN, fd, V, mC, q, ad, eA, eW, ib, aT, cy, eV, dL, hu, ae, eL, pl, d, s, iU, aW, Y, ff, eZ, r, alb, af, en, g, cm, iK, bz, Ye, rR, eM, iT, dB, bE, cj, fL, m, WL, Vj, B); #c1(XRCCGBPI) c2(NP_150592) c3(mitochondrial inner membrane protease ATP23 homolog) c4(228110) c5(228111, 228112, 228113, 228114, 228115, 228116) c6( ) c7(d, eA, qy, Hp); #c1(XRN1) c2(NP_001269786) c3(5′-3′ exoribonuclease 1 isoform c) c4(102765, 102766) c5(102767, 102768, 102769, 102770, 102771, 102772, 102773, 102774, 102775, 102776, 102777, 102778) c6( ) c7(en, c, ff, K, aiM); #c1(XRN1) c2(NP_001269788) c3(5′-3′ exoribonuclease 1 isoform d) c4(102790, 102791) c5(102792, 102793, 102794, 102795, 102796, 102797, 102798, 102799, 102800, 102801, 102802, 102803) c6( ) c7(en, c, ff, K, aiM); #c1(XRN1) c2(NP_061874) c3(5′-3′ exoribonuclease 1 isoform a) c4(200874) c5(200875, 200876, 200877, 200878, 200879, 200880) c6( ) c7(en, c, ff, K, aiM); #c1(XRN2) c2(NP_036387) c3(5′-3′ exoribonuclease 2) c4(173474, 173475) c5(173476, 173477, 173478, 173479, 173480, 173481, 173482, 173483, 173484, 173485, 173486, 173487, 173488, 173489, 173490, 173491, 173492) c6( ) c7(av, bz); #c1(XRRA1) c2(NP_001257309) c3(X-ray radiation resistance-associated protein 1 isoform 2) c4(95125) c5(95126, 95127, 95128, 95129, 95130, 95131) c6( ) c7(T); #c1(XRRA1) c2(NP_892014) c3(X-ray radiation resistance-associated protein 1 isoform 1) c4(257597, 257598) c5(257599, 257600, 257601, 257602, 257603, 257604, 257605, 257606, 257607, 257608, 257609, 257610) c6( ) c7(T); #c1(XRRA1) c2(XP_011543058) c3(X-ray radiation resistance-associated protein 1 isoform X3) c4(341855, 341856) c5(341857, 341858, 341859, 341860, 341861, 341862, 341863, 341864, 341865, 341866, 341867, 341868) c6( ) c7(T); #c1(XRRA1) c2(XP_011543062) c3(X-ray radiation resistance-associated protein 1 isoform X7) c4(341869) c5( ) c6( ) c7(T); #c1(XRRA1) c2(XP_011543066) c3(X-ray radiation resistance-associated protein 1 isoform X11) c4( ) c5(341870, 341871, 341872, 341873, 341874, 341875) c6( ) c7(T); #c1(XXYLT1) c2(NP_689744) c3(xyloside xylosyltransferase 1) c4(243832, 243833, 243834) c5(243835, 243836, 243837, 243838, 243839, 243840) c6( ) c7(BM); #c1(XXYLT1) c2(XP_011510752) c3(xyloside xylosyltransferase 1 isoform X3) c4( ) c5(301158, 301159, 301160, 301161, 301162, 301163, 301164, 301165, 301166, 301167, 301168, 301169, 301170, 301171, 301172, 301173) c6( ) c7(eM); #c1(XYLB) c2(NP_005099) c3(xylulose kinase) c4(152860, 152861) c5(152862, 152863, 152864, 152865, 152866, 152867, 152868, 152869, 152870, 152871, 152872, 152873) c6( ) c7(M, EQ); #c1(XYLT1) c2(NP_071449) c3(xylosyltransferase 1 precursor) c4(211739) c5(211740, 211741, 211742, 211743, 211744, 211745) c6( ) c7(A, bf, oe, fO, bm, cz, fo, dy, lB, fq); #c1(XYLT2) c2(NP_071450) c3(xylosyltransferase 2) c4(211746, 211747) c5(211748, 211749, 211750, 211751, 211752, 211753, 211754, 211755, 211756, 211757, 211758, 211759, 211760, 211761, 211762, 211763) c6( ) c7(A, oe, cz, fo, dy, fL, qa); #c1(XYLT2) c2(XP_011523416) c3(xylosyltransferase 2 isoform X2) c4( ) c5(317942) c6( ) c7(A, De, cz, fo, dy, fL, qa); #c1(YAE1D1) c2(NP_001269375) c3(yae1 domain-containing protein 1 isoform 2) c4(101346) c5(101347, 101348, 101349, 101350, 101351, 101352) c6( ) c7(ac); #c1(YAE1D1) c2(NP_064577) c3(yae1 domain-containing protein 1 isoform 1) c4(203059) c5(203060, 203061, 203062, 203063, 203064, 203065) c6( ) c7(ac); #c1(YAP1) c2(NP_001123617) c3(yorkie homolog isoform 1) c4(47873) c5(47874, 47875, 47876, 47877, 47878, 47879, 47880) c6( ) c7(Mb, T, aZ, if, aw, ht, aA, cz, bB, ci, qN, q, ad, ip, ft, hd, ib, aE, jz, nv, hb, cVi, aT, K, hu, ae, af, kl, Ar, Jl, aW, Y, ff, eH, r, aq, aF, en, eF, t, iK, bz, Qt, iR, iP, ck, kn, iO, Ew, Dy, eM, nB, bE, wR, np, m, it, B, hS); #c1(YAP1) c2(NP_001181973) c3(yorkie homolog isoform 3) c4(74453) c5(74454, 74455, 74456, 74457, 74458, 74459) c6( ) c7(Mb, T, aZ, if, aw, ht, aA, cz, bB, ci, qN, q, ad, ip, ft, hd, ib, aE, jz, nv, hb, cVi, aT, K, hu, ae, af, kl, Or, Jl, aW, Y, ff, eH, r, aq, aF, en, eF, t, iK, bz, Qt, iR, iP, ck, kn, iO, Ew, Dy, eM, nB, bE, wR, np, m, it, B, hS); #c1(YAP1) c2(NP_001181974) c3(yorkie homolog isoform 4) c4(74460) c5(74461, 74462, 74463, 74464, 74465) c6( ) c7(Mb, T, aZ, if, aw, ht, aA, cz, bB, ci, qN, q, ad, ip, ft, hd, ib, aE, jz, nv, hb, cVi, aT, K, hu, ae, af, kl, Or, Jl, aW, Y, ff, eH, r, aq, aF, en, eF, t, iK, bz, Qt, iR, iP, ck, kn, iO, Ew, Dy, eM, nB, bE, wR, np, m, it, B, hS); #c1(YAP1) c2(NP_001269027) c3(yorkie homolog isoform 5) c4(100266) c5(100267, 100268, 100269, 100270, 100271, 100272) c6( ) c7(Mb, T, aZ, if, aw, ht, aA, cz, bB, ci, qN, q, ad, ip, ft, hd, ib, aE, jz, nv, hb, cVi, aT, K, hu, ae, af, kl, Qr, Jl, aW, Y, ff, eH, r, aq, aF, en, eF, t, iK, bz, Qt, iR, iP, ck, kn, iO, Ew, Dy, eM, nB, bE, wR, np, m, it, B, hS); #c1(YAP1) c2(NP_001269029) c3(yorkie homolog isoform 8) c4(100273) c5(100274, 100275, 100276, 100277, 100278, 100279) c6( ) c7(Mb, T, aZ, if, aw, ht, aA, cz, bB, ci, qN, q, ad, ip, ft, hd, ib, aE, jz, nv, hb, cVi, aT, K, hu, ae, af, kl, Or, Jl, aW, Y, ff, eH, r, aq, aF, en, eF, t, iK, bz, Qt, iR, iP, ck, kn, iO, Ew, Dy, eM, nB, bE, wR, np, m, it, B, hS); #c1(YAP1) c2(NP_006097) c3(yorkie homolog isoform 2) c4(161859) c5(161860, 161861, 161862, 161863, 161864, 161865, 161866, 161867) c6( ) c7(Mb, T, aZ, if, aw, ht, aA, cz, bB, ci, qN, q, ad, ip, ft, hd, ib, aE, jz, nv, hb, cVi, aT, K, hu, ae, af, kl, Or, Jl, aW, Y, ff, eH, r, aq, af, en, eF, t, iK, bz, Qt, iR, iP, ck, kn, iO, Ew, Dy, eM, nB, bE, wR, np, m, it, B, hS); #c1(YAP1) c2(XP_005271437) c3 (transcriptional coactivator YAP1 isoform X2) c4(283397) c5(283398, 283399, 283400, 283401, 283402, 283403) c6( ) c7(Mb, T, aZ, if, aw, ht, aA, cz, bB, ci, qN, q, ad, ip, ft, hd, ib, aE, jz, nv, hb, cVi, aT, K, hu, ae, af, kl, Or, Jl, aW, Y, ff, eH, r, aq, aF, en, eF, t, iK, bz, Qt, iR, iP, ck, kn, iO, Ew, Dy, eM, nB, bE, wR, np, m, it, B, hS); #c1(YARS2) c2(NP_001035526) c3(tyrosine-tRNA ligase, mitochondrial precursor) c4(32853) c5(32854, 32855, 32856, 32857, 32858, 32859) c6( ) c7(kA, oj, aNG, fu, bU, cVj, cxH); #c1(YBX2) c2(NP_057066) c3(Y-box-binding protein 2) c4(188743) c5(188744, 188745, 188746, 188747, 188748, 188749, 188750, 188751, 188752) c6(188753, 188754) c7(gU, ND, wu, aMv, vH, wm, Le); #c1(YBX3) c2(NP_001138898) c3(Y-box-binding protein 3 isoform b) c4(58108) c5(58109, 58110, 58111, 58112, 58113, 58114) c6( ) c7(Hw, T, py, avS, aW, Pg, jh, aT, cy, bt, B); #c1(YBX3) c2(NP_003642) c3(Y-box-binding protein 3 isoform a) c4(139852) c5(139853, 139854, 139855, 139856, 139857, 139858, 139859) c6( ) c7(Hw, T, py, avS, aW, Pg, jh, aT, cy, bt, B); #c1(YDJC) c2(NP_001017964) c3(UPF0249 protein ydjC homolog) c4(26677) c5(26678, 26679, 26680, 26681, 26682, 26683) c6( ) c7(hp, cr); #c1(YDJC) c2(XP_011528208) c3(UPF0249 protein ydjC homolog isoform X1) c4(323301) c5(323302, 323303, 323304, 323305, 323306, 323307) c6( ) c7(hp, cr); #c1(YEATS4) c2(NP_006521) c3(YEATS domain-containing protein 4 isoform 1) c4(165970) c5(165971, 165972, 165973, 165974, 165975, 165976, 165977) c6( ) c7(d, eM, pN, au, cz, as, bz, fs, eA); #c1(YES1) c2(XP_011524038) c3(tyrosine-protein kinase Yes isoform X1) c4(318621) c5(318622, 318623, 318624, 318625, 318626, 318627, 318628, 318629, 318630, 318631, 318632, 318633, 318634, 318635) c6( ) c7(T, d, aE, bE, Y, aw, aT, aA, bB, h, aF, ey, K, B); #c1(YIFI1) c2(NP_065203) c3(protein YIFI1 isoform 1) c4(204575) c5(204576, 204577, 204578, 204579, 204580, 204581) c6( ) c7(aq); #c1(YIPF1) c2(XP_011539907) c3(protein YIPF1 isoform X3) c4(338191) c5(338192, 338193, 338194, 338195, 338196) c6( ) c7(bt); #c1(YIPF3) c2(NP_056203) c3(protein YIPF3) c4(186311) c5(186312, 186313, 186314, 186315, 186316, 186317) c6( ) c7(cUW, fr); #c1(YIPF5) c2(NP_001020118) c3(protein YIPF5 isoform a) c4(28238, 28239, 28240, 28241) c5(28242, 28243, 28244, 28245, 28246, 28247, 28248, 28249, 28250, 28251, 28252, 28253, 28254, 28255, 28256, 28257, 28258, 28259, 28260, 28261, 28262, 28263, 28264, 28265, 28266) c6( ) c7(ey, hp); #c1(YIPF5) c2(NP_001258661) c3(protein YIPF5 isoform b) c4(96289, 96290) c5(96291, 96292, 96293, 96294, 96295, 96296, 96297, 96298, 96299, 96300, 96301, 96302, 96303, 96304, 96305) c6( ) c7(ey, hp); #c1(YKT6) c2(NP_006546) c3(synaptobrevin homolog YKT6) c4(166142, 166143) c5(166144, 166145, 166146, 166147, 166148, 166149, 166150, 166151, 166152, 166153, 166154, 166155) c6( ) c7(aT); #c1(YLPM1) c2(NP_062535) c3(YLP motif-containing protein 1) c4(201812, 201813) c5(201814, 201815, 201816, 201817, 201818, 201819, 201820, 201821, 201822, 201823, 201824, 201825, 201826, 201827, 201828, 201829, 201830, 201831) c6( ) c7(AT, aW, aT); #c1(YLPM1) c2(XP_011535268) c3(YLP motif-containing protein 1 isoform X2) c4(332170) c5( ) c6( ) c7(AT, aW, aT); #c1(YLPM1) c2(XP_011535269) c3(YLP motif-containing protein 1 isoform X3) c4( ) c5(332171, 332172) c6( ) c7(AT, aW, aT); #c1(YME1L1) c2(NP_001240795) c3(ATP-dependent zinc metalloprotease YME1L1 isoform 4) c4(88821, 88822) c5(88823, 88824, 88825, 88826, 88827, 88828, 88829, 88830, 88831, 88832, 88833, 88834) c6( ) c7(y, ji, F, K, Ht); #c1(YME1L1) c2(NP_055078) c3(ATP-dependent zinc metalloprotease YME1L1 isoform 3) c4(178817, 178818, 178819) c5(178820, 178821, 178822, 178823, 178824, 178825, 178826, 178827, 178828, 178829, 178830, 178831, 178832, 178833, 178834, 178835, 178836) c6( ) c7(y, ji, F, K, Ht); #c1(YME1L1) c2(NP_647473) c3(ATP-dependent zinc metalloprotease YME1L1 isoform 1) c4(238620, 238621) c5(238622, 238623, 238624, 238625, 238626, 238627, 238628, 238629, 238630, 238631, 238632, 238633, 238634) c6( ) c7(y, ji, F, K, Ht); #c1(YPEL1) c2(NP_037445) c3(protein yippee-like 1) c4(175551) c5(175552, 175553, 175554, 175555, 175556, 175557) c6( ) c7(aT); #c1(YPEL2) c2(NP_001005404) c3(protein yippee-like 2) c4(21640) c5(21641, 21642, 21643, 21644, 21645, 21646) c6( ) c7(aW, aT); #c1(YPEL3) c2(NP_001138996) c3(protein yippee-like 3 isoform 2) c4( ) c5(58578, 58579, 58580, 58581, 58582) c6( ) c7(bE, aW, aT, bB, j, g, t, X); #c1(YPEL3) c2(NP_113665) c3(protein yippee-like 3 isoform 1) c4(222017, 222018) c5(222019, 222020, 222021, 222022, 222023, 222024, 222025) c6( ) c7(bE, aW, aT, bB, j, g, t, X); #c1(YPEL4) c2(NP_659445) c3(protein yippee-like 4) c4(239939, 239940) c5(239941, 239942, 239943, 239944, 239945, 239946, 239947, 239948, 239949, 239950, 239951, 239952, 239953, 239954, 239955, 239956, 239957, 239958, 239959, 239960, 239961, 239962, 239963, 239964) c6( ) c7(aT); #c1(YPEL5) c2(NP_001120872) c3(protein yippee-like 5) c4(44893) c5(44894, 44895, 44896, 44897, 44898, 44899, 44900, 44901, 44902, 44903, 44904, 44905, 44906, 44907, 44908, 44909, 44910, 44911, 44912, 44913, 44914, 44915) c6( ) c7(bX, aT); #c1(YTHDC1) c2(NP_001026902) c3(YTH domain-containing protein 1 isoform 1) c4(29944) c5(29945, 29946, 29947, 29948, 29949, 29950, 29951, 29952, 29953, 29954) c6( ) c7(T, Fw, hL, cl); #c1(YTHDC1) c2(NP_588611) c3(YTH domain-containing protein 1 isoform 2) c4(234799) c5(234800, 234801, 234802, 234803, 234804, 234805, 234806, 234807, 234808, 234809, 234810, 234811, 234812, 234813, 234814, 234815, 234816) c6( ) c7(T, Fw, hL, cl); #c1(YTHDC2) c2(NP_073739) c3(probable ATP-dependent RNA helicase YTHDC2) c4(213360) c5(213361, 213362, 213363, 213364, 213365, 213366, 213367, 213368, 213369) c6( ) c7(qb); #c1(YTHDC2) c2(XP_011541886) c3(probable ATP-dependent RNA helicase YTHDC2 isoform X4) c4(340419) c5(340420, 340421, 340422, 340423, 340424) c6( ) c7(qb); #c1(YWHAB) c2(NP_647539) c3(14-3-3 protein beta/alpha) c4(238709, 238710, 238711) c5(238712, 238713, 238714, 238715, 238716, 238717, 238718, 238719, 238720, 238721, 238722, 238723, 238724, 238725, 238726, 238727, 238728, 238729, 238730, 238731, 238732, 238733) c6( ) c7(iu, T, hl, eZ, aA, cj, GC, aF, eA, eu, bN, dS); #c1(YWHAE) c2(NP_006752) c3(14-3-3 protein epsilon) c4(167717, 167718) c5(167719, 167720, 167721, 167722, 167723, 167724, 167725, 167726, 167727, (67728, 167729, 167730) c6( ) c7(tJ, T, Js, qx, fd, qy, fZ, aA, n, ol, bS, aeu, bB, oY, fr, abQ, hF, eA, br, ad, x, oB, av, iu, cc, jZ, oW, aT, aU, dV, dz, vw, bK, h, jj, bdS, afX, pq, Nr, afy, oP, aix, hG, aW, Y, et, gK, iU, FD, pr, Mz, kD, po, aF, jh, pm, bw, aiv, bN, Nq, yd, ck, pc, UY, iA, bE, iT, kz, FB, bt, cj, pg, dW, biJ, k, eg, Th, aEc, B, Jo); #c1(YWHAG) c2(NP_036611) c3(14-3-3 protein gamma) c4(174819, 174820, 174821) c5(174822, 174823, 174824, 174825, 174826, 174827, 174828, 174829, 174830, 174831, 174832, 174833, 174834, 174835, 174836, 174837, 174838, 174839) c6( ) c7(av, alC, blw, op, aW, vA, aT, nA, hf, f, V, bz, iu, aR, eK); #c1(YWHAH) c2(NP_003396) c3(14-3-3 protein eta) c4(138163) c5(138164, 138165, 138166, 138167, 138168, 138169) c6( ) c7(T, cc, d, iT, an, aT, ti, bk, ey, bq, he, ik); #c1(YWHAQ) c2(NP_006817) c3(14-3-3 protein theta) c4(168347, 168348, 168349) c5(168350, 168351, 168352, 168353, 168354, 168355, 168356, 168357, 168358, 168359, 168360, 168361, 168362, 168363, 168364, 168365, 168366, 168367, 168368, 168369, 168370, 168371) c6( ) c7(av, Hx, aaF, dt, fR, c, WY, aav, aaG, U, aT, cj, eZ, OY, bl, act, af, aR, dS); #c1(YWHAZ) c2(NP_001129172) c3(14-3-3 protein zeta/delta) c4(50988, 50989, 50990, 50991, 50992, 50993, 50994, 50995, 50996, 50997, 50998) c5(50999, 51000, 51001, 51002, 51003, 51004, 51005, 51006, 51007, 51008, 51009, 51010, 51011, 51012, 51013, 51014, 51015, 51016, 51017, 51018, 51019, 51020, 51021, 51022, 51023, 51024, 51025, 51026, 51027, 51028, 51029, 51030, 51031, 51032, 51033, 51034, 51035, 51036, 51037, 51038, 51039, 51040, 51041, 51042, 51043, 51044, 51045, 51046, 51047, 51048, 51049, 51050, 51051, 51052, 51053, 51054, 51055, 51056, 51057, 51058, 51059, 51060, 51061, 51062, 51063, 51064, 51065, 51066, 51067, 51068, 51069, 51070, 51071, 51072, 51073, 51074, 51075, 51076, 51077, 51078, 51079, 51080, 51081, 51082, 51083, 51084, 51085, 51086, 51087, 51088, 51089, 51090, 51091, 51092, 51093, 51094, 51095, 51096, 51097, 51098, 51099, 51100) c6( ) c7(DS, bM, T, ea, e, al, bB, ci, auu, aR, gJ, K, zQ, Jl, aW, zE, cm, eM, U, m, Ro, NK, dD, Ht, aw, n, aj, zP, ai, Yc, ey, aq, ky, cA, at, B, eR, dk, aA, cz, fF, v, j, C, V, q, J, jF, axa, bUp, aT, bZk, cy, an, ae, af, ne, Pw, kl, s, ev, Zt, aAm, r, akY, en, t, iK, bN, cT, Yq, c, mf, EY, aan, dG, eq, qi, ad, av, ji, cb, ff, aF, iV, bz, bF, gk, bE, z, X, HN, aS, y, xg, iu); #c1(YWHAZ) c2(XP_011515592) c3(14-3-3 protein zeta/delta isoform X2) c4( ) c5(308420) c6( ) c7(DS, bM, T, ea, e, al, bB, ci, auu, aR, gJ, K, zQ, Jl, aW, zE, cm, eM, U, m, Ro, NK, dD, Ht, aw, n, aj, zP, ai, Yc, ey, aq, ky, cA, at, B, eR, dk, aA, cz, fF, v, j, C, V, q, J, jF, axa, bUp, aT, bZk, cy, an, ae, af, ne, Pw, kl, s, ev, Zt, aAm, r, akY, en, t, iK, bN, cT, Yq, c, mf, EY, aan, dG, eq, qi, ad, av, ji, cb, ff, aF, iV, bz, bF, gk, bE, z, X, HN, aS, y, xg, iu); #c1(YY1) c2(NP_003394) c3(transcriptional repressor protein YY1) c4(138148, 138149) c5(138150, 138151, 138152, 138153, 138154, 138155, 138156, 138157, 138158, 138159, 138160, 138161, 138162) c6( ) c7(bL, bM, T, bfY, if, iW, jh, cz, bB, fd, aj, bS, bck, ci, nA, fu, dG, bfU, v, hF, q, J, cd, ad, hX, eA, ip, mj, ib, x, pp, iX, aql, yg, kH, aT, h, vc, hn, H, RG, ae, A, iU, xR, aW, Y, eH, bfV, r, eZ, kp, hp, eF, t, fr, bz, akG, bfW, bw, hq, ck, qJ, BU, cA, avS, bE, iT, aDs, cj, bfX, k, m, pw, y, bt, Mj, B, EB, L); #c1(YY1AP1) c2(NP_001185832) c3(YY1-associated protein 1 isoform 6) c4(76487, 76488) c5(76489, 76490, 76491, 76492, 76493, 76494, 76495, 76496, 76497, 76498, 76499, 76500) c6( ) c7(T, aw, aA, cz, bB, q, ad, jz, aE, aT, ae, aW, Y, r, aq, aF, iR, t, Qt, iP, W, Dy, eM, bE, m, it, B, hS); #c1(YY1AP1) c2(NP_001185833) c3(YY1-associated protein 1 isoform 7) c4(76501, 76502, 76503) c5(76504, 76505, 76506, 76507, 76508, 76509, 76510, 76511) c6( ) c7(T, aw, aA, cz, bB, q, ad, jz, aE, aT, ae, aW, Y, r, aq, aF, iR, t, Qt, iP, W, Dy, eM, bE, m, it, B, hS); #c1(YY1AP1) c2(NP_001185834) c3(YY1-associated protein 1 isoform 8) c4( ) c5(76512, 76513, 76514, 76515, 76516, 76517, 76518, 76519, 76520, 76521, 76522, 76523, 76524, 76525, 76526, 76527, 76528, 76529, 76530, 76531, 76532) c6( ) c7(T, aw, aA, cz, bB, q, ad, jz, aE, aT, ae, aW, Y, r, aq, aF, iR, t, Qt, iP, W, Dy, eM, bE, m, it, B, hS); #c1(YY1AP1) c2(NP_001185835) c3(YY1-associated protein 1 isoform 9) c4( ) c5(76533, 76534, 76535, 76536, 76537, 76538, 76539, 76540, 76541, 76542, 76543, 76544, 76545, 76546, 76547, 76548, 76549, 76550, 76551, 76552, 76553, 76554, 76555, 76556, 76557, 76558, 76559, 76560, 76561, 76562) c6( ) c7(T, aw, aA, cz, bB, q, ad, jz, aE, al, ae, aW, Y, r, aq, aF, iR, t, Qt, iP, W, Dy, eM, bE, m, it, B, hS); #c1(YY1AP1) c2(NP_620829) c3(YY1-associated protein 1 isoform 2) c4(238129, 238130, 238131, 238132, 238133, 238134, 238135) c5(238136, 238137, 238138, 238139, 238140, 238141) c6( ) c7(T, aw, aA, cz, bB, q, ad, jz, aE, aT, ae, aW, Y, r, aq, aF, iR, t, Qt, iP, W, Dy, eM, bE, m, it, B, hS); #c1(YY1AP1) c2(NP_620832) c3(YY1-associated protein 1 isoform 5) c4( ) c5(238142) c6( ) c7(T, aw, aA, cz, bB, q, ad, jz, aE, aT, ae, aW, Y, r, aq, af, iR, t, Qt, iP, W, Dy, eM, bE, m, it, B, hS); #c1(YY1AP1) c2(XP_011508023) c3(YY1-associated protein 1 isoform X2) c4(298455, 298456) c5(298457, 298458, 298459, 298460, 298461, 298462, 298463, 298464, 298465, 298466, 298467, 298468, 298469, 298470, 298471, 298472, 298473) c6( ) c7(T, aw, aA, cz, bB, q, ad, jz, aE, aT, ae, aW, Y, r, aq, aF, iR, t, Qt, iP, W, Dy, eM, bE, m, it, B, hS); #c1(YY1AP1) c2(XP_011508024) c3(YY1-associated protein 1 isoform X1) c4( ) c5(298474, 298475, 298476, 298477, 298478, 298479, 298480, 298481, 298482) c6( ) c7(T, aw, aA, cz, bB, q, ad, jz, aE, aT, ae, aW, Y, r, aq, aF, iR, t, Qt, iP, W, Dy, eM, bE, m, it, B, hS); #c1(ZACN) c2(NP_851321) c3(zinc-activated ligand-gated ion channel precursor) c4(254548, 254549) c5(254550, 254551, 254552, 254553, 254554, 254555, 254556, 254557, 254558, 254559, 254560, 254561) c6( ) c7(bL, T, sS, mh, ZH, s, CN, yo, aW, lW, ff, mO, aT, hR, dG, m, ci, en, t, iK, JW); #c1(ZAK) c2(NP_598407) c3(mitogen-activated protein kinase kinase kinase MLT isoform 2) c4(235517, 235518, 235519) c5(235520, 235521, 235522, 235523, 235524, 235525, 235526, 235527, 235528, 235529, 235530, 235531, 235532, 235533, 235534, 235535, 235536, 235537, 235538, 235539, 235540, 235541) c6( ) c7(x, asc, d, fM, ac, aE, bE, jh, cj, aT, n, bB, kp, SZ, k, h, fd, bt, bz, cd); #c1(ZAK) c2(XP_005246697) c3(mitogen-activated protein kinase kinase kinase MLT isoform X1) c4(267360) c5(267361, 267362, 267363, 267364, 267365, 267366, 267367, 267368, 267369, 267370) c6( ) c7(x, asc, d, fM, ac, aE, bE, jh, cj, aT, n, bB, kp, SZ, k, h, fd, bt, bz, cd); #c1(ZAP70) c2(NP_001070) c3(tyrosine-protein kinase ZAP-70 isoform 1) c4(35264, 35265, 35266) c5( ) c6( ) c7(GI, T, fM, dA, bRe, if, aDT, eX, jh, oY, Sv, ci, qN, ai, aHT, ad, eA, x, fX, h, eV, By, hn, baJ, cVk, Xb, akV, cVl, iU, rh, aHW, oV, Of, eZ, Og, as, aBs, Cv, ck, pc, AV, HW, cj, amP, k, aDb, y); #c1(ZAP70) c2(NP_997402) c3(tyrosine-protein kinase ZAP-70 isoform 2) c4( ) c5(265524, 265525, 265526, 265527, 265528) c6( ) c7(GI, T, fM, dA, bRe, if, aDT, ex, jh, oY, Sv, ci, qN, ai, aHT, ad, eA, x, fX, h, eV, By, hn, baJ, cVk, Xb, akV, cVl, iU, rh, aHW, oV, Of, eZ, Og, as, aBs, Cv, ck, pc, AV, HW, cj, amP, k, aDb, y); #c1(ZARI) c2(NP_783318) c3(zygote arrest protein 1) c4(251621) c5(251622, 251623, 251624, 251625, 251626, 251627, 251628, 251629) c6( ) c7(dB, T, if, Y, qN); #c1(ZAR1L) c2(NP_001130043) c3(ZARI-like protein) c4(52478, 52479) c5(52480, 52481, 52482, 52483, 52484, 52485) c6( ) c7(aW, aT); #c1(ZAR1L) c2(XP_011533503) c3(ZARI-like protein isoform X1) c4( ) c5(329590, 329591, 329592, 329593, 329594, 329595) c6( ) c7(aW, aT); #c1(ZASP) c2(NP_001276862) c3(Z0-2 associated speckle protein) c4( ) c5(108509) c6( ) c7(Ws, cc, K, oj, mx); #c1(ZBED1) c2(NP_001164606) c3(zinc finger BED domain-containing protein 1) c4(67052, 67053) c5(67054, 67055, 67056, 67057, 67058, 67059, 67060, 67061, 67062, 67063, 67064, 67065, 67066, 67067, 67068, 67069, 67070, 67071, 67072, 67073) c6( ) c7(T, aep, cB, ci, K, g, EZ, ae, eW, ad, afu); #c1(ZBED4) c2(NP_055653) c3(zinc finger BED domain-containing protein 4) c4(182546) c5(182547, 182548, 182549, 182550, 182551, 182552) c6( ) c7(uJ, ey); #c1(ZBED5) c2(NP_001137139) c3(zinc finger BED domain-containing protein 5) c4(54962) c5(54963, 54964, 54965, 54966, 54967, 54968, 54969, 54970, 54971, 54972) c6( ) c7(bL, fq, EQ, cz); #c1(ZBP1) c2(NP_001153890) c3(Z-DNA-binding protein 1 isoform c) c4(61019) c5(61020, 61021, 61022, 61023, 61024, 61025) c6( ) c7(T, aW, eZ, aT, j, fL, Ea, pb, mC); #c1(ZBP1) c2(NP_001153891) c3(Z-DNA-binding protein 1 isoform d) c4(61026) c5(61027, 61028, 61029, 61030, 61031, 61032) c6( ) c7(T, aW, eZ, aT, j, fL, Ea, pb, mC); #c1(ZBP1) c2(NP_110403) c3(Z-DNA-binding protein 1 isoform a) c4(220081, 220082, 220083) c5(220084, 220085, 220086, 220087, 220088, 220089, 220090, 220091, 220092, 220093, 220094, 220095, 220096, 220097, 220098, 220099, 220100, 220101) c6( ) c7(T, aW, eZ, aT, j, fL, Ea, pb, mC); #c1(ZBP1) c2(XP_011527359) c3(Z-DNA-binding protein 1 isoform X1) c4(321977) c5(321978, 321979, 321980, 321981, 321982, 321983) c6( ) c7(T, aW, eZ, aT, j, fL, Ea, pb, mC); #c1(ZBTB10) c2(NP_001099009) c3(zinc finger and BTB domain-containing protein 10 isoform a) c4(41630) c5(41631, 41632, 41633, 41634, 41635, 41636, 41637, 41638, 41639, 41640) c6( ) c7(aW, gT, aF, aA, aT); #c1(ZBTB10) c2(NP_001264074) c3(zinc finger and BTB domain-containing protein 10 isoform c) c4(97493) c5(97494, 97495, 97496, 97497, 97498, 97499) c6( ) c7(aW, gT, aF, aA, aT); #c1(ZBTB10) c2(NP_076418) c3(zinc finger and BTB domain-containing protein 10 isoform b) c4(214112) c5(214113, 214114, 214115, 214116, 214117, 214118) c6( ) c7(aW, gT, aF, aA, aT); #c1(ZBTB12) c2(NP_862825) c3(zinc finger and BTB domain-containing protein 12) c4( ) c5(255772, 255773, 255774, 255775, 255776, 255777, 255778, 255779, 255780, 255781, 255782, 255783, 255784, 255785, 255786, 255787, 255788) c6( ) c7(A, W, cr, eZ, V, B); #c1(ZBTB12) c2(XP_011512685) c3(zinc finger and BTB domain-containing protein 12 isoform X1) c4(303693) c5(303694, 303695, 303696) c6( ) c7(A, W, cr, eZ, V, B); #c1(ZBTB14) c2(NP_001137295) c3(zinc finger and BTB domain-containing protein 14) c4(55323) c5(55324, 55325, 55326, 55327, 55328, 55329, 55330, 55331, 55332, 55333, 55334) c6( ) c7(Be); #c1(ZBTB16) c2(NP_001018011) c3(zinc finger and BTB domain-containing protein (6) c4(26826, 26827) c5(26828, 26829, 26830, 26831, 26832, 26833, 26834, 26835, 26836, 26837, 26838, 26839, 26840, 26841, 26842, 26843, 26844) c6( ) c7(iW, bO, qN, q, ad, cVm, jj, ae, pi, cp, iU, Y, r, lo, t, ck, ac, zG, m, bD, y, bt, kS); #c1(ZBTB16) c2(XP_005271715) c3(zinc finger and BTB domain-containing protein 16 isoform X2) c4(283558, 283559) c5(283560, 283561, 283562, 283563, 283564, 283565, 283566, 283567, 283568, 283569, 283570, 283571) c6( ) c7(iW, bO, qN, q, ad, cVm, jj, ae, pi, cp, iU, Y, r, lo, t, ck, ac, zG, m, bD, y, bt, kS); #c1 (ZBTB17) c2(XP_011540387) c3(zinc finger and BTB domain-containing protein 17 isoform X3) c4(338888) c5( ) c6( ) c7(T, fM, mx, jh, cm, K, m, t, AN); #c1(ZBTB17) c2(XP_011540389) c3(zinc finger and BTB domain-containing protein 17 isoform X4) c4( ) c5(338889, 338890, 338891, 338892, 338893, 338894) c6( ) c7(T, fM, mx, jh, cm, K, m, t, AN); #c1(ZBTB17) c2(XP_011540390) c3(zinc finger and BTB domain-containing protein 17 isoform X1) c4(338895) c5(338896, 338897, 338898, 338899, 338900, 338901, 338902, 338903, 338904, 338905, 338906, 338907, 338908, 338909, 338910, 338911) c6( ) c7(T, fM, mx, jh, cm, K, m, t, AN); #c1(ZBTB18) c2(NP_991331) c3(zinc finger and BTB domain-containing protein 18 isoform 1) c4(263431, 263432) c5(263433, 263434, 263435, 263436, 263437, 263438, 263439, 263440, 263441, 263442, 263443, 263444, 263445, 263446) c6( ) c7(jf, dB, cVn, uz, cVo, bp); #c1(ZBTB2) c2(NP_065912) c3(zinc finger and BTB domain-containing protein 2) c4(206196) c5(206197, 206198, 206199, 206200, 206201, 206202, 206203) c6( ) c7(aA, aF, q, r); #c1(ZBTB20) c2(NP_001157814) c3(zinc finger and BTB domain-containing protein 20 isoform 1) c4(62720, 62721, 62722) c5(62723, 62724, 62725, 62726, 62727, 62728, 62729, 62730, 62731, 62732, 62733, 62734, 62735, 62736, 62737, 62738, 62739, 62740, 62741, 62742) c6( ) c7(dY, eM, cVp, aA, n, aq, aF, iR, bz, B); #c1(ZBTB20) c2(NP_001157817) c3(zinc finger and BTB domain-containing protein 20 isoform 2) c4(62743) c5(62744, 62745, 62746, 62747, 62748, 62749, 62750, 62751, 62752, 62753, 62754, 62755, 62756, 62757, 62758, 62759, 62760, 62761, 62762, 62763, 62764, 62765, 62766, 62767, 62768) c6( ) c7(dY, eM, cVp, aA, n, aq, aF, iR, bz, B); #c1(ZBTB21) c2(NP_001091872) c3(zinc finger and BTB domain-containing protein 21 isoform L) c4(38741, 38742) c5(38743, 38744, 38745, 38746, 38747, 38748, 38749, 38750, 38751, 38752, 38753, 38754, 38755, 38756, 38757, 38758, 38759, 38760, 38761, 38762, 38763, 38764, 38765) c6( ) c7(f, j); #c1(ZBTB21) c2(NP_001091873) c3(zinc finger and BTB domain-containing protein 21 isoform S) c4(38766, 38767) c5(38768, 38769, 38770, 38771, 38772, 38773, 38774, 38775, 38776, 38777, 38778, 38779, 38780, 38781) c6( ) c7(f, j); #c1(ZBTB22) c2(NP_001138810) c3(zinc finger and BTB domain-containing protein 22) c4(57789, 57790, 57791) c5(57792, 57793, 57794, 57795, 57796, 57797, 57798, 57799, 57800, 57801, 57802, 57803, 57804, 57805, 57806, 57807, 57808, 57809, 57810, 57811, 57812, 57813, 57814, 57815, 57816, 57817, 57818, 57819, 57820, 57821, 57822, 57823, 57824, 57825, 57826, 57827, 57828, 57829, 57830, 57831, 57832, 57833, 57834, 57835, 57836, 57837, 57838, 57839, 57840, 57841, 57842, 57843, 57844, 57845, 57846, 57847, 57848, 57849, 57850, 57851, 57852) c6( ) c7(eZ); #c1(ZBTB24) c2(NP_055612) c3(zinc finger and BTB domain-containing protein 24 isoform 1) c4(182275) c5(182276, 182277, 182278, 182279, 182280, 182281) c6( ) c7(T, aW, aT, aq, cVq, nA, B); #c1(ZBTB32) c2(NP_055198) c3(zinc finger and BTB domain-containing protein 32) c4(179722) c5(179723, 179724, 179725, 179726, 179727, 179728, 179729, 179730, 179731, 179732) c6( ) c7(oZ, os, j, kS); #c1(ZBTB33) c2(NP_006768) c3(transcriptional regulator Kaiso) c4(167889) c5(167890, 167891, 167892, 167893, 167894, 167895, 167896, 167897, 167898, 167899, 167900, 167901, 167902, 167903, 167904, 167905, 167906, 167907) c6( ) c7(T, X, g); #c1(ZBTB34) c2(NP_001092740) c3(zinc finger and BTB domain-containing protein 34) c4( ) c5(39593, 39594, 39595, 39596, 39597) c6( ) c7(aE); #c1(ZBTB34) c2(XP_005252046) c3(zinc finger and BTB domain-containing protein 34 isoform X1) c4(271501) c5(271502, 271503) c6( ) c7(aE); #c1(ZBTB34) c2(XP_011517001) c3(zinc finger and BTB domain-containing protein 34 isoform X2) c4(310481) c5(310482, 310483, 310484, 310485, 310486) c6( ) c7(aE); #c1(ZBTB38) c2(NP_001073881) c3(zinc finger and BTB domain-containing protein 38) c4(36220, 36221, 36222, 36223, 36224, 36225, 36226) c5(36227, 36228, 36229, 36230, 36231, 36232, 36233, 36234, 36235, 36236, 36237, 36238, 36239, 36240, 36241, 36242, 36243, 36244, 36245, 36246, 36247, 36248, 36249, 36250, 36251, 36252, 36253, 36254, 36255, 36256, 36257, 36258, 36259, 36260, 36261, 36262, 36263, 36264, 36265, 36266, 36267, 36268, 36269, 36270, 36271, 36272, 36273, 36274, 36275, 36276, 36277, 36278, 36279) c6( ) c7(xb, dt, ad, dS); #c1(ZBTB38) c2(XP_005247313) c3(zinc finger and BTB domain-containing protein 38 isoform X3) c4(267790) c5(267791, 267792, 267793, 267794, 267795, 267796, 267797) c6( ) c7(xb, dt, ad, dS); #c1(ZBTB38) c2(XP_011510912) c3(zinc finger and BTB domain-containing protein 38 isoform X2) c4(301370, 301371, 301372, 301373) c5(301374, 301375, 301376, 301377, 301378, 301379, 301380, 301381, 301382, 301383, 301384, 301385, 301386, 301387, 301388, 301389, 301390) c6( ) c7(xb, dt, ad, dS); #c1(ZBTB4) c2(NP_065950) c3(zinc finger and BTB domain-containing protein 4) c4(206457) c5(206458, 206459, 206460, 206461, 206462, 206463, 206464, 206465, 206466, 206467, 206468, 206469, 206470) c6( ) c7(aW, aE, ae, ad, aT); #c1(ZBTB41) c2(NP_919290) c3(zinc finger and BTB domain-containing protein 41) c4(258479) c5(258480, 258481, 258482, 258483, 258484, 258485) c6( ) c7(W); #c1(ZBTB46) c2(XP_005260254) c3(zinc finger and BTB domain-containing protein 46 isoform X1) c4(276486) c5(276487, 276488, 276489, 276490, 276491, 276492, 276493, 276494, 276495, 276496, 276497, 276498, 276499, 276500) c6( ) c7(V, eA); #c1(ZBTB48) c2(XP_011539612) c3(zinc finger and BTB domain-containing protein 48 isoform X1) c4(337854) c5( ) c6( ) c7(cm, T); #c1(ZBTB48) c2(XP_011539614) c3(zinc finger and BTB domain-containing protein 48 isoform X2) c4( ) c5(337855, 337856, 337857, 337858, 337859, 337860) c6( ) c7(cm, T); #c1(ZBTB49) c2(NP_660334) c3(zinc finger and BTB domain-containing protein 49) c4(240986, 240987) c5(240988, 240989, 240990, 240991, 240992, 240993, 240994, 240995, 240996, 240997, 240998, 240999) c6( ) c7(K); #c1(ZBTB49) c2(XP_005248008) c3(zinc finger and BTB domain-containing protein 49 isoform X3) c4( ) c5(268174, 268175, 268/76, 268/77, 268178, 268179, 268180) c6( ) c7(K); #c1(ZBTB49) c2(XP_011511718) c3(zinc finger and BTB domain-containing protein 49 isoform X2) c4(302440, 302441) c5(302442, 302443, 302444, 302445, 302446, 302447) c6( ) c7(K); #c1(ZBTB5) c2(NP_055687) c3(zinc finger and BTB domain-containing protein 5) c4(182775) c5(182776, 182777, 182778, 182779, 182780, 182781) c6( ) c7(bOL, bP); #c1(ZBTB7C) c2(NP_001034449) c3(zinc finger and BTB domain-containing protein 7C) c4( ) c5(31724, 31725, 31726, 31727, 31728, 31729, 31730, 31731) c6( ) c7(bL, es, eR, iA, h, bE, e, bB, dq, aj, mf, j, dG, v, ei, qN, aY, GR, J, ip); #c1(ZBTB7C) c2(XP_011524163) c3(zinc finger and BTB domain-containing protein 7C isoform X2) c4(318717) c5(318718, 318719, 318720, 318721, 318722, 318723, 318724, 318725, 318726, 318727, 318728) c6( ) c7(bL, es, eR, iA, h, bE, e, bB, dq, aj, mf, j, dG, v, ei, qN, aY, GR, J, ip); #c1(ZBTB7C) c2(XP_011524171) c3(zinc finger and BTB domain-containing protein 7C isoform X4) c4(318729, 318730) c5(318731, 318732, 318733) c6( ) c7(bL, es, eR, iA, h, bE, e, bB, dq, aj, mf, j, dG, v, ei, qN, aY, GR, J, ip); #c1(ZBTB9) c2(NP_689948) c3(zinc finger and BTB domain-containing protein 9) c4(244474) c5(244475, 244476, 244477, 244478, 244479, 244480, 244481, 244482, 244483, 244484) c6( ) c7(at, eZ); #c1(ZC2HC1B) c2(NP_001013645) c3(zinc finger C2HC domain-containing protein 1B) c4(25646, 25647, 25648, 25649) c5(25650, 25651, 25652, 25653, 25654, 25655, 25656, 25657, 25658, 25659, 25660, 25661, 25662, 25663, 25664, 25665, 25666, 25667, 25668, 25669, 25670, 25671, 25672, 25673) c6( ) c7(ac); #c1(ZC3H10) c2(NP_116175) c3(zinc finger CCCH domain-containing protein 10) c4(225586, 225587, 225588, 225589) c5(225590, 225591, 225592, 225593, 225594, 225595, 225596, 225597, 225598, 225599, 225600, 225601, 225602, 225603, 225604, 225605, 225606, 225607, 225608, 225609, 225610, 225611, 225612, 225613) c6( ) c7(pb); #c1(ZC3H11A) c2(NP_055642) c3(zinc finger CCCH domain-containing protein 11A) c4(182465, 182466, 182467) c5(182468, 182469, 182470, 182471, 182472, 182473, 182474, 182475, 182476, 182477, 182478, 182479, 182480, 182481, 182482, 182483, 182484, 182485, 182486, 182487, 182488, 182489, 182490, 182491, 182492, 182493, 182494, 182495, 182496, 182497, 182498, 182499, 182500) c6( ) c7(aW, cz, Be, aT); #c1(ZC3H11A) c2(XP_011508506) c3(zinc finger CCCH domain-containing protein 11A isoform X2) c4(299218) c5(299219, 299220, 299221, 299222, 299223, 299224, 299225) c6( ) c7(aW, cz, Be, aT); #c1(ZC3H12C) c2(NP_203748) c3(probable ribonuclease ZC3H12C) c4(228489) c5(228490, 228491, 228492, 228493, 228494, 228495) c6( ) c7(dh, cr); #c1(ZC3H12D) c2(NP_997243) c3(probable ribonuclease ZC3H12D) c4(265268, 265269, 265270) c5(265271, 265272, 265273, 265274, 265275, 265276, 265277, 265278, 265279, 265280, 265281, 265282, 265283, 265284, 265285, 265286, 265287, 265288, 265289, 265290, 265291, 265292) c6( ) c7(bF, bE, jh, bB, bC, bD, bz); #c1(ZC3H14) c2(NP_001153575) c3(zinc finger CCCH domain-containing protein 14 isoform 5) c4(60672) c5(60673, 60674, 60675, 60676, 60677, 60678) c6( ) c7(Hx); #c1(ZC3H14) c2(NP_001153576) c3(zinc finger CCCH domain-containing protein 14 isoform 6) c4(60679) c5(60680, 60681, 60682, 60683, 60684, 60685) c6( ) c7(Hx); #c1(ZC3H14) c2(NP_079100) c3(zinc finger CCCH domain-containing protein 14 isoform 1) c4(217016) c5(217017, 217018, 217019, 217020, 217021, 217022) c6( ) c7(Hx); #c1(ZC3H14) c2(NP_997543) c3(zinc finger CCCH domain-containing protein 14 isoform 2) c4(265586) c5(265587, 265588, 265589, 265590, 265591, 265592) c6( ) c7(Hx); #c1(ZC3H14) c2(NP_997544) c3(zinc finger CCCH domain-containing protein 14 isoform 3) c4(265593, 265594) c5(265595, 265596, 265597, 265598, 265599, 265600, 265601, 265602) c6( ) c7(Hx); #c1(ZC3H14) c2(NP_997545) c3(zinc finger CCCH domain-containing protein 14 isoform 4) c4(265603, 265604) c5(265605, 265606, 265607, 265608, 265609, 265610, 265611, 265612, 265613, 265614, 265615, 265616) c6( ) c7(Hx); #c1(ZC3H14) c2(XP_005268124) c3(zinc finger CCCH domain-containing protein 14 isoform X1) c4(281579) c5(281580, 281581, 281582, 281583, 281584, 281585) c6( ) c7(Hx); #c1(ZC3H14) c2(XP_005268125) c3(zinc finger CCCH domain-containing protein 14 isoform X2) c4(281586, 281587) c5(281588, 281589, 281590, 281591, 281592, 281593, 281594, 281595, 281596, 281597, 281598, 281599) c6( ) c7(Hx); #c1(ZC3H14) c2(XP_005268126) c3(zinc finger CCCH domain-containing protein 14 isoform X5) c4(281600) c5(281601, 281602) c6( ) c7(Hx); #c1(ZC3H14) c2(XP_005268127) c3(zinc finger CCCH domain-containing protein 14 isoform X6) c4(281603, 281604, 281605) c5(281606, 281607, 281608, 281609, 281610, 281611) c6( ) c7(Hx); #c1(ZC3H14) c2(XP_011535464) c3(zinc finger CCCH domain-containing protein 14 isoform X8) c4( ) c5(332504, 332505, 332506, 332507, 332508, 332509, 332510, 332511, 332512, 332513, 332514, 332515, 332516, 332517, 332518, 332519, 332520, 332521, 332522, 332523, 332524, 332525, 332526, 332527) c6( ) c7(Hx); #c1(ZC3H15) c2(NP_060941) c3(zinc finger CCCH domain-containing protein 15) c4(198806, 198807, 198808) c5(198809, 198810, 198811, 198812, 198813, 198814, 198815, 198816, 198817, 198818, 198819, 198820, 198821, 198822, 198823, 198824, 198825, 198826, 198827, 198828, 198829, 198830, 198831, 198832) c6( ) c7(ck, bt); #c1(ZC3H15) c2(XP_011509787) c3(zinc finger CCCH domain-containing protein 15 isoform X1) c4(300347) c5( ) c6( ) c7(ck, bt); #c1(ZC3H3) c2(NP_055932) c3(zinc finger CCCH domain-containing protein 3) c4(184512) c5(184513, 184514, 184515, 184516, 184517) c6( ) c7(ac); #c1(ZC3H3) c2(XP_006716599) c3(zinc finger CCCH domain-containing protein 3 isoform X2) c4( ) c5(290419) c6( ) c7(ac); #c1(ZC3H4) c2(NP_055983) c3(zinc finger CCCH domain-containing protein 4) c4(184877) c5(184878, 184879, 184880, 184881, 184882, 184883) c6( ) c7(fq); #c1(ZC3H4) c2(XP_011524973) c3(zinc finger CCCH domain-containing protein 4 isoform X7) c4( ) c5(319463) c6( ) c7(fq); #c1(ZC3H7A) c2(NP_054872) c3(zinc finger CCCH domain-containing protein 7A) c4(178133) c5(178134, 178135, 178136, 178137, 178138, 178139, 178140, 178141, 178142, 178143) c6( ) c7(Th, T); #c1(ZC3H7B) c2(NP_060060) c3(zinc finger CCCH domain-containing protein 78) c4(193937) c5(193938, 193939, 193940, 193941, 193942, 193943, 193944, 193945, 193946, 193947) c6( ) c7(Th, BD); #c1(ZC3HAV1) c2(NP_064504) c3(zinc finger CCCH-type antiviral protein 1 isoform 1) c4(202657, 202658, 202659) c5(202660, 202661, 202662, 202663, 202664, 202665, 202666) c6( ) c7(AT, aW, aT, h, V, hX); #c1(ZC3HAV1) c2(NP_078901) c3(zinc finger CCCH-type antiviral protein 1 isoform 2) c4( ) c5(215992, 215993, 215994, 215995, 215996, 215997, 215998, 215999, 216000, 216001, 216002) c6( ) c7(AT, aW, aT, h, V, hX); #c1(ZC3HC1) c2(NP_001269119) c3(nuclear-interacting partner of ALK isoform 2) c4(100481) c5(100482, 100483, 100484, 100485, 100486, 100487) c6( ) c7(ea, dD, j, y, eq); #c1(ZC3HC1) c2(NP_001269120) c3(nuclear-interacting partner of ALK isoform 3) c4(100488) c5(100489, 100490, 100491, 100492, 100493, 100494) c6( ) c7(ea, dD, j, y, eq); #c1(ZC3HC1) c2(NP_057562) c3(nuclear-interacting partner of ALK isoform 1) c4(191363) c5(191364, 191365, 191366, 191367, 191368, 191369) c6( ) c7(ea, dD, j, y, eq); #c1(ZC4H2) c2(NP_001171504) c3(zinc finger C4H2 domain-containing protein isoform 3) c4(70327) c5(70328, 70329, 70330, 70331, 70332, 70333) c6( ) c7(lf, oR, cVr, nA); #c1(ZC4H2) c2(NP_001230733) c3(zinc finger C4H2 domain-containing protein isoform 2) c4( ) c5(86740, 86741, 86742, 86743, 86744) c6( ) c7(lf, oR, cVr, nA); #c1(ZC4H2) c2(NP_061154) c3 (zinc finger C4H2 domain-containing protein isoform 1) c4(199615, 199616, 199617) c5(199618, 199619, 199620, 199621, 199622, 199623, 199624, 199625, 199626, 199627, 199628, 199629, 199630) c6( ) c7(lf, oR, cVr, nA); #c1(ZCCHC11) c2(XP_011539402) c3(terminal uridylyltransferase 4 isoform X11) c4(337515) c5( ) c6( ) c7(T); #c1(ZCCHC11) c2(XP_011539404) c3(terminal uridylyltransferase 4 isoform X14) c4( ) c5(337516, 337517, 337518, 337519, 337520, 337521, 337522, 337523, 337524, 337525, 337526, 337527) c6( ) c7(T); #c1(ZCCHC12) c2(NP_776159) c3(zinc finger CCHC domain-containing protein 12) c4(250877) c5(250878, 250879, 250880, 250881, 250882, 250883) c6( ) c7(nf); #c1(ZCCHC14) c2(NP_055959) c3(zinc finger CCHC domain-containing protein 14) c4(184700) c5( ) c6( ) c7(awX); #c1(ZCCHC14) c2(XP_005255915) c3(zinc finger CCHC domain-containing protein 14 isoform X1) c4( ) c5(273895, 273896, 273897, 273898, 273899, 273900) c6( ) c7(awX); #c1(ZCCHC2) c2(NP_060212) c3(zinc finger CCHC domain-containing protein 2) c4(194768) c5(194769, 194770, 194771, 194772, 194773, 194774, 194775) c6( ) c7(ey, ds, cd, y); #c1(ZCCHC3) c2(NP_149080) c3(zinc finger CCHC domain-containing protein 3) c4(227048) c5(227049, 227050, 227051, 227052, 227053, 227054) c6( ) c7(bK); #c1(ZCCHC6) c2(NP_001171988) c3(terminal uridylyltransferase 7 isoform 1) c4(72123, 72124) c5(72125, 72126, 72127, 72128, 72129, 72130, 72131, 72132, 72133, 72134, 72135, 72136, 72137, 72138, 72139, 72140, 72141, 72142, 72143, 72144, 72145, 72146, 72147, 72148, 72149) c6( ) c7(bA); #c1(ZCCHC6) c2(NP_001172003) c3(terminal uridylyltransferase 7 isoform 2) c4(72171, 72172, 72173) c5(72174, 72175, 72176, 72177, 72178, 72179) c6( ) c7(bA); #c1(ZCCHC6) c2(XP_005252265) c3(terminal uridylyltransferase 7 isoform X6) c4(271634) c5( ) c6( ) c7(bA); #c1(ZCCHC6) c2(XP_005252266) c3(terminal uridylyltransferase 7 isoform X7) c4(271635) c5(271636, 271637, 271638, 271639, 271640, 271641, 271642, 271643, 271644, 271645, 271646, 271647, 271648, 271649, 271650, 271651, 271652) c6( ) c7(bA); #c1(ZCCHC6) c2(XP_0067 (7346) c3(terminal uridylyltransferase 7 isoform X2) c4(290960, 290961) c5( ) c6( ) c7(bA); #c1(ZCCHC8) c2(NP_060082) c3(zinc finger CCHC domain-containing protein 8) c4(194056, 194057) c5(194058, 194059, 194060, 194061, 194062, 194063, 194064, 194065, 194066, 194067, 194068, 194069, 194070) c6( ) c7(nA); #c1(ZCCHC8) c2(XP_011536857) c3(zinc finger CCHC domain-containing protein 8 isoform X3) c4(334547) c5(334548, 334549, 334550, 334551, 334552, 334553, 334554, 334555, 334556) c6( ) c7(nA); #c1(ZCRB1) c2(NP_149105) c3(zinc finger CCHC-type and RNA-binding motif-containing protein 1) c4(227178) c5(227179, 227180, 227181, 227182, 227183, 227184) c6( ) c7(B, cVs); #c1(ZCWPW1) c2(NP_001244937) c3(zinc finger CW-type PWWP domain protein 1 isoform 2) c4(92342) c5(92343, 92344, 92345, 92346, 92347, 92348) c6( ) c7(W); #c1(ZCWPW1) c2(NP_060454) c3(zinc finger CW-type PWWP domain protein 1 isoform 1) c4(196025) c5(196026, 196027, 196028, 196029, 196030, 196031) c6( ) c7(W); #c1(ZCWPW1) c2(XP_005250536) c3(zinc finger CW-type PWWP domain protein 1 isoform X5) c4( ) c5(270367, 270368, 270369, 270370, 270371, 270372) c6( ) c7(W); #c1(ZCWPW1) c2(XP_006716099) c3(zinc finger CW-type PWWP domain protein 1 isoform X1) c4(290036) c5( ) c6( ) c7(W); #c1(ZCWPW1) c2(XP_006716100) c3(zinc finger CW-type PWWP domain protein 1 isoform X2) c4(290037) c5(290038, 290039, 290040, 290041, 290042, 290043) c6( ) c7(W); #c1(ZCWPW1) c2(XP_006716103) c3(zinc finger CW-type PWWP domain protein 1 isoform X4) c4( ) c5(290044, 290045, 290046, 290047, 290048, 290049) c6( ) c7(W); #c1(ZDBF2) c2(NP_065974) c3(DBF4-type zinc finger-containing protein 2 isoform 1) c4(206577) c5(206578, 206579, 206580, 206581, 206582, 206583) c6( ) c7(ft); #c1(ZDHHC1) c2(NP_037436) c3(probable palmitoyltransferase ZDHHC1) c4(175522) c5(175523, 175524, 175525, 175526, 175527, 175528, 175529) c6( ) c7(bL, y, A, j, cz); #c1(ZDHHC1) c2(XP_005255972) c3(probable palmitoyltransferase ZDHHC1 isoform X6) c4( ) c5(273909) c6( ) c7(bL, y, A, j, cz); #c1(ZDHHC11) c2(NP_079062) c3(probable palmitoyltransferase ZDHHC11) c4(216852) c5(216853, 216854, 216855, 216856, 216857, 216858, 216859, 216860, 216861, 216862) c6( ) c7(eL, dL); #c1(ZDHHC12) c2(NP_116188) c3(probable palmitoyltransferase ZDHHC12) c4(225628, 225629) c5(225630, 225631, 225632, 225633, 225634, 225635, 225636, 225637, 225638, 225639, 225640, 225641, 225642, 225643, 225644, 225645, 225646, 225647, 225648, 225649, 225650, 225651) c6( ) c7(rR); #c1(ZDHHC12) c2(XP_011517418) c3(probable palmitoyltransferase ZDHHC12 isoform X2) c4( ) c5(311130, 311131, 311132, 311133, 311134, 311135, 311136, 311137) c6( ) c7(rR); #c1(ZDHHC13) c2(NP_001001483) c3(palmitoyltransferase ZDHHC13 isoform 2) c4(18508) c5( ) c6( ) c7(bA, aeM, rR, ac, R); #c1(ZDHHC13) c2(NP_061901) c3(palmitoyltransferase ZDHHC13 isoform 1) c4(201127) c5(201128, 201129, 201130, 201131, 201132, 201133, 201134, 201135, 201136, 201137, 201138, 201139) c6( ) c7(bA, aeM, rR, ac, R); #c1(ZDHHC14) c2(NP_078906) c3(probable palmitoyltransferase ZDHHC14 isoform 1) c4(216046, 216047) c5(216048, 216049, 216050, 216051, 216052, 216053, 216054, 216055, 216056, 216057, 216058, 216059) c6( ) c7(T, ae, ad); #c1(ZDHHC14) c2(NP_714968) c3(probable palmitoyltransferase ZDHHC14 isoform 2) c4(246670) c5(246671, 246672, 246673, 246674, 246675, 246676) c6( ) c7(T, ae, ad); #c1(ZDHHC15) c2(NP_001139728) c3(palmitoyltransferase ZDHHC15 isoform 2) c4(59768) c5(59769, 59770, 59771, 59772, 59773, 59774) c6( ) c7(nf, cVt, rR, rY); #c1(ZDHHC15) c2(NP_659406) c3 (palmitoyltransferase ZDHHC15 isoform 1) c4(239763) c5(239764, 239765, 239766, 239767, 239768, 239769, 239770) c6( ) c7(nf, cVt, rR, rY); #c1(ZDHHC15) c2(XP_011529179) c3(palmitoyltransferase ZDHHC15 isoform X2) c4(324580) c5(324581, 324582, 324583, 324584, 324585) c6( ) c7(nf, cVt, rR, rY); #c1(ZDHHC17) c2(NP_056151) c3 (palmitoyltransferase ZDHHC17) c4(185976) c5(185977, 185978, 185979, 185980, 185981, 185982, 185983, 185984, 185985, 185986) c6( ) c7(rR); #c1(ZDHHC17) c2(XP_005268808) c3(palmitoyltransferase ZDHHC17 isoform X5) c4(282103) c5(282104, 282105, 282106, 282107, 282108, 282109) c6( ) c7(rR); #c1(ZDHHC2) c2(NP_057437) c3(palmitoyltransferase ZDHHC2) c4(190794) c5(190795, 190796, 190797, 190798, 190799, 190800) c6( ) c7(T, JI, Y, aA, aq, aF, y, B); #c1(ZDHHC7) c2(NP_001139020) c3(palmitoyltransferase ZDHHC7 isoform 1) c4(58668) c5(58669, 58670, 58671, 58672, 58673, 58674) c6( ) c7(aW); #c1(ZDHHC7) c2(NP_060210) c3(palmitoyltransferase ZDHHC7 isoform 2) c4(194761) c5(194762, 194763, 194764, 194765, 194766, 194767) c6( ) c7(aW); #c1(ZDHHC8) c2(NP_037505) c3(probable palmitoyltransferase ZDHHC8 isoform 2) c4(175889, 175890) c5(175891, 175892, 175893, 175894, 175895, 175896, 175897, 175898, 175899, 175900, 175901, 175902) c6( ) c7(agd, ib, RX, an, agY, hS); #c1(ZDHHC8) c2(XP_006724302) c3(probable palmitoyltransferase ZDHHC8 isoform X1) c4(295897) c5(295898, 295899, 295900, 295901, 295902, 295903) c6( ) c7(agd, ib, RX, an, agY, hS); #c1(ZDHHC9) c2(NP_001008223) c3(palmitoyltransferase ZDHHC9) c4(22897, 22898, 22899) c5(22900, 22901, 22902, 22903, 22904, 22905, 22906, 22907, 22908, 22909, 22910, 22911, 22912, 22913, 22914, 22915) c6( ) c7(T, ev, cVu, r, q, g, X); #c1(ZDHHC9) c2(XP_011529649) c3(palmitoyltransferase ZDHHC9 isoform X1) c4( ) c5(325298, 325299, 325300, 325301, 325302, 325303) c6( ) c7(T, ev, cVu, r, q, g, X); #c1(ZEB1) c2(NP_001121600) c3(zinc finger E-box-binding homeobox 1 isoform a) c4(45832, 45833, 45834) c5(45835, 45836, 45837, 45838, 45839, 45840, 45841, 45842, 45843, 45844, 45845, 45846, 45847) c6( ) c7(T, os, aSP, zX, cVw, bou, jh, aA, bB, n, oZ, it, j, ci, q, ad, iu, ft, hd, aOu, hw, hW, yt, ib, aE, jz, aT, ab, dL, rB, hB, ae, eL, Lu, d, kj, je, aW, Y, ev, eH, ff, eZ, r, Qm, kp, aF, en, cl, eF, t, bz, cVv, iM, kP, eM, nB, bE, bmD, X, hL, m, all, B, hS); #c1(ZEB1) c2(NP_001167564) c3(zinc finger E-box-binding homeobox 1 isoform c) c4(69279) c5(69280, 69281, 69282, 69283, 69284, 69285) c6( ) c7(T, os, aSP, zX, cVw, bou, jh, aA, bB, n, oZ, it, j, ci, q, ad, iu, ft, hd, aOu, hw, hW, yt, ib, aE, jz, aT, ab, dL, rB, hB, ae, eL, Lu, d, kj, je, aW, Y, ev, eH, ff, eZ, r, Qm, kp, aF, en, cl, eF, t, bz, cVv, iM, kP, eM, nB, bE, bmD, X, hL, m, all, B, hS); #c1(ZEB1) c2(NP_001167566) c3(zinc finger E-box-binding homeobox 1 isoform e) c4(69286) c5(69287, 69288, 69289, 69290, 69291, 69292) c6( ) c7(T, os, aSP, zX, cVw, bou, jh, aA, bB, n, oZ, it, j, ci, q, ad, iu, ft, hd, aOu, hw, hW, yt, ib, aE, jz, aT, ab, dL, rB, hB, ae, eL, Lu, d, kj, je, aW, Y, ev, eH, ff, eZ, r, Qm, kp, aF, en, cl, eF, t, bz, cVv, iM, kP, eM, nB, bE, bmD, X, hL, m, all, B, hS); #c1(ZEB1) c2(NP_001167567) c3(zinc finger E-box-binding homeobox 1 isoform f) c4(69293, 69294) c5(69295, 69296, 69297, 69298, 69299, 69300, 69301, 69302, 69303, 69304, 69305, 69306, 69307, 69308, 69309, 69310, 69311, 69312, 69313, 69314, 69315, 69316, 69317, 69318) c6( ) c7(T, os, aSP, zX, cVw, bou, jh, aA, bB, n, oZ, it, j, ci, q, ad, iu, ft, hd, aOu, hw, hW, yt, ib, aE, jz, aT, ab, dL, rB, hB, ae, eL, Lu, d, kj, je, aW, Y, ev, eH, ff, eZ, r, Qm, kp, aF, en, cl, eF, t, bz, cVv, iM, kP, eM, nB, bE, bmD, X, hL, m, all, B, hS); #c1(ZEB1) c2(NP_110378) c3(zinc finger E-box-binding homeobox 1 isoform b) c4(219774, 219775, 219776) c5(219777, 219778, 219779, 219780, 219781, 219782, 219783, 219784, 219785, 219786, 219787, 219788, 219789, 219790, 219791, 219792, 219793, 219794, 219795) c6( ) c7(T, os, aSP, zX, cVw, bou, jh, aA, bB, n, oZ, it, j, ci, q, ad, iu, ft, hd, aOu, hw, hW, yt, ib, aE, jz, aT, ab, dL, rB, hB, ae, eL, Lu, d, kj, je, aW, Y, ev, eH, ff, eZ, r, Qm, kp, aF, en, cl, eF, t, bz, cVv, iM, kP, eM, nB, bE, bmD, X, hL, m, all, B, hS); #c1(ZEB1) c2(XP_011517950) c3 (zinc finger E-box-binding homeobox 1 isoform X1) c4(311955, 311956) c5(311957, 311958, 311959, 311960, 311961) c6( ) c7(T, os, aSP, zX, cVw, bou, jh, aA, bB, n, oZ, it, j, ci, q, ad, iu, ft, hd, aOu, hw, hW, yt, ib, aE, jz, aT, ab, dL, rB, hB, ae, eL, Lu, d, kj, je, aW, Y, ev, eH, ff, eZ, r, Qm, kp, af, en, cl, eF, t, bz, cVv, iM, kP, eM, nB, bE, bmD, X, hL, m, all, B, hS); #c1(ZEB2) c2(NP_001165124) c3(zinc finger E-box-binding homeobox 2 isoform 2) c4(67490) c5(67491, 67492, 67493, 67494, 67495, 67496, 67497, 67498, 67499, 67500, 67501, 67502, 67503) c6( ) c7(T, aAf, bou, fG, Ol, aA, bB, bD, cQ, fr, ci, bjN, nA, q, ad, eA, ft, ib, aE, hW, yt, Tf, aT, kf, h, dL, ahE, ae, Gc, eL, aW, Y, ff, eH, r, aq, Pp, bSZ, ahl, en, ir, Z, t, eF, aF, iK, all, ck, es, eM, iA, bE, Be, m, Wl, it, B, hS); #c1(ZEB2) c2(NP_055610) c3(zinc finger E-box-binding homeobox 2 isoform 1) c4(182219, 182220, 182221, 182222, 182223, 182224, 182225) c5(182226, 182227, 182228, 182229, 182230, 182231, 182232, 182233, 182234, 182235, 182236, 182237, 182238, 182239, 182240, 182241, 182242, 182243, 182244, 182245, 182246, 182247, 182248, 182249, 182250, 182251, 182252, 182253, 182254, 182255, 182256, 182257, 182258, 182259, 182260, 182261, 182262, 182263, 182264, 182265, 182266, 182267, 182268, 182269) c6( ) c7(T, aAf, bou, fG, Ol, aA, bB, bD, cQ, fr, ci, bjN, nA, q, ad, eA, ft, ib, aE, hW, yt, Tf, aT, kf, h, dL, ahE, ae, Gc, eL, aW, Y, ff, eH, r, aq, Pp, bSZ, ahl, en, ir, Z, t, eF, aF, iK, all, ck, es, eM, iA, bE, Be, m, Wl, it, B, hS); #c1(ZFAND3) c2(NP_068762) c3(AN1-type zinc finger protein 3) c4(210258) c5(210259, 210260, 210261, 210262, 210263, 210264, 210265) c6( ) c7(bL); #c1(ZFAND5) c2(NP_001095891) c3(AN1-type zinc finger protein 5) c4(40899, 40900) c5(40901, 40902, 40903, 40904, 40905, 40906, 40907, 40908, 40909, 40910, 40911, 40912, 40913, 40914, 40915, 40916, 40917, 40918, 40919, 40920, 40921, 40922, 40923, 40924, 40925) c6( ) c7(ad); #c1(ZFAND6) c2(NP_001229846) c3(AN1-type zinc finger protein 6 isoform b) c4(85403) c5(85404, 85405, 85406, 85407, 85408) c6( ) c7(bL, A, cz, awj, j, lQ, Pk, y); #c1(ZFAND6) c2(NP_061879) c3(AN1-type zinc finger protein 6 isoform a) c4(200897) c5(200898, 200899, 200900, 200901, 200902, 200903, 200904, 200905, 200906, 200907, 200908, 200909, 200910, 200911, 200912, 200913, 200914, 200915, 200916, 200917) c6( ) c7(bL, A, cz, awj, j, lQ, Pk, y); #c1(ZFAND6) c2(XP_011519985) c3(AN1-type zinc finger protein 6 isoform X2) c4( ) c5(314841, 314842, 314843, 314844, 314845, 314846, 314847, 314848) c6( ) c7(bL, A, cz, awj, j, lQ, Pk, y); #c1(ZFAT) c2(NP_001025110) c3(zinc finger protein ZFAT isoform 2) c4(29332) c5(29333, 29334, 29335, 29336, 29337, 29338, 29339, 29340, 29341, 29342, 29343, 29344, 29345, 29346) c6( ) c7(bL, A, Lj, cz, hD, cLn, hz, j, y, yF); #c1(ZFAT) c2(NP_001167628) c3(zinc finger protein ZFAT isoform 3) c4(69457) c5(69458, 69459, 69460, 69461, 69462, 69463) c6( ) c7(bL, A, Lj, cz, hD, cLn, hz, j, y, yF); #c1(ZFAT) c2(NP_001167629) c3(zinc finger protein ZFAT isoform 4) c4(69464) c5(69465, 69466, 69467, 69468, 69469, 69470) c6( ) c7(bL, A, Lj, cz, hD, cLn, hz, j, y, yF); #c1(ZFAT) c2(NP_065914) c3(zinc finger protein ZFAT isoform 1) c4(206204) c5(206205, 206206, 206207, 206208, 206209, 206210, 206211, 206212, 206213, 206214) c6( ) c7(bL, A, Lj, cz, hD, cLn, hz, j, y, yF); #c1(ZFAT) c2(XP_011515506) c3(zinc finger protein ZFAT isoform X2) c4(308358) c5( ) c6( ) c7(bL, A, Lj, cz, hD, cLn, hz, j, y, yF); #c1(ZFC3H1) c2(NP_659419) c3(zinc finger C3HI domain-containing protein) c4(239799, 239800) c5(239801, 239802, 239803, 239804, 239805, 239806, 239807, 239808, 239809, 239810, 239811, 239812) c6( ) c7(j); #c1(ZFHX2) c2(XP_006720345) c3(zinc finger homeobox protein 2 isoform X5) c4( ) c5(293502) c6( ) c7(fq); #c1(ZFHX2) c2(XP_011535551) c3(zinc finger homeobox protein 2 isoform X1) c4(332609, 332610, 332611) c5(332612, 332613, 332614, 332615, 332616, 332617, 332618, 332619, 332620, 332621, 332622, 332623, 332624, 332625, 332626, 332627, 332628, 332629, 332630) c6( ) c7(fq); #c1(ZFHX2) c2(XP_011535552) c3(zinc finger homeobox protein 2 isoform X2) c4(332631, 332632) c5( ) c6( ) c7(fq); #c1(ZFHX2) c2(XP_011535553) c3(zinc finger homeobox protein 2 isoform X3) c4( ) c5(332633, 332634, 332635, 332636, 332637, 332638, 332639, 332640, 332641, 332642, 332643, 332644) c6( ) c7(fq); #c1(ZFHX2) c2(XP_011535554) c3(zinc finger homeobox protein 2 isoform X4) c4( ) c5(332645) c6( ) c7(fq); #c1(ZFHX3) c2(NP_001158238) c3(zinc finger homeobox protein 3 isoform B) c4(63491) c5(63492, 63493, 63494, 63495, 63496) c6( ) c7(T, mr, wL, if, e, aw, aA, aj, j, qN, ad, b, M, aT, ae, eu, aW, aq, aF, pT, cm, ik, fq, ac, i, lf, B); #c1(ZFHX3) c2(NP_008816) c3(zinc finger homeobox protein 3 isoform A) c4(168778) c5(168779, 168780, 168781, 168782, 168783, 168784, 168785) c6( ) c7(T, mr, wL, if, e, aw, aA, aj, j, qN, ad, b, M, aT, ae, eu, aW, aq, aF, pT, cm, iK, fq, ac, i, lf, B); #c1(ZFHX4) c2(XP_011515897) c3(zinc finger homeobox protein 4 isoform X1) c4(309002, 309003, 309004) c5(309005, 309006, 309007, 309008, 309009, 309010, 309011, 309012, 309013, 309014, 309015, 309016, 309017, 309018, 309019, 309020, 309021, 309022, 309023, 309024, 309025, 309026, 309027, 309028, 309029, 309030, 309031, 309032, 309033, 309034, 309035, 309036, 309037, 309038, 309039) c6( ) c7(iC, KG, cVx, d, eA); #c1(ZFHX4) c2(XP_011515898) c3(zinc finger homeobox protein 4 isoform X2) c4(309040, 309041, 309042, 309043) c5(309044, 309045, 309046, 309047, 309048, 309049, 309050, 309051, 309052, 309053, 309054, 309055, 309056, 309057, 309058, 309059, 309060, 309061, 309062, 309063) c6( ) c7(iC, KG, cVx, d, eA); #c1(ZFHX4) c2(XP_011515899) c3(zinc finger homeobox protein 4 isoform X3) c4(309064) c5(309065) c6( ) c7(iC, KG, cVx, d, eA); #c1(ZFP1) c2(XP_011521223) c3(zinc finger protein 1 homolog isoform X2) c4( ) c5(316143, 316144, 316145) c6( ) c7(bP, T, ae, ad, bA); #c1(ZFP30) c2(NP_055713) c3(zinc finger protein 30 homolog) c4(182994) c5(182995, 182996, 182997, 182998, 182999, 183000, 183001, 183002, 183003, 183004, 183005, 183006, 183007, 183008) c6( ) c7(ac); #c1(ZFP30) c2(XP_011524942) c3(zinc finger protein 30 homolog isoform X4) c4(319424) c5(319425, 319426, 319427, 319428, 319429, 319430, 319431) c6( ) c7(ac); #c1(ZFP36) c2(NP_003398) c3(tristetraprolin) c4(138170) c5(138171, 138172, 138173, 138174, 138175, 138/76, 138177, 138178) c6( ) c7(T, DM, dD, eR, Ke, ea, if, DV, jh, bB, aj, bS, j, ci, v, eq, qN, ai, J, ad, hX, eA, ke, eW, qI, aT, h, DO, DG, j, d, s, aW, ev, gC, dN, aq, lo, iR, cM, ik, ck, DT, bE, dh, aeM, ei, y, kS, B); #c1(ZFP36L1) c2(NP_001231630) c3(zinc finger protein 36, C3HI type-like 1 isoform 2) c4(87360, 87361) c5(87362, 87363, 87364, 87365) c6( ) c7(x, Do, aW, aT, hn, hp, m, k, cl, g, V, aiJ); #c1(ZFP36LI) c2(NP_004917) c3(zinc finger protein 36, C3H1 type-like 1 isoform 1) c4( ) c5(150762, 150763, 150764, 150765, 150766, 150767, 150768, 150769, 150770, 150771, 150772, 150773, 150774) c6( ) c7(x, Do, aW, aT, hn, hp, m, k, cl, g, V, aiJ); #c1(ZFP36L2) c2(NP_008818) c3(zinc finger protein 36, C3H1 type-like 2) c4(168808) c5(168809, 168810, 168811, 168812, 168813, 168814, 168815, 168816) c6( ) c7(ck, nJ, T, x, CN, eM, Qu, aW, ff, jh, aT, hn, k, en, hF, bt, ad); #c1(ZFP37) c2(NP_001269444) c3(zinc finger protein 37 homolog isoform 1) c4(101676) c5(101677, 101678, 101679, 101680, 101681, 101682) c6( ) c7(cvZ, K, cVy, atb); #c1(ZFP37) c2(NP_001269447) c3(zinc finger protein 37 homolog isoform 2) c4(101689) c5(101690, 101691, 101692, 101693, 101694, 101695) c6( ) c7(cvZ, K, cVy, atb); #c1(ZFP37) c2(NP_003399) c3(zinc finger protein 37 homolog isoform 3) c4(138/79) c5(138180, 138181, 138182, 138183, 138184, 138185, 138186) c6( ) c7(cvZ, K, cVy, atb); #c1(ZFP42) c2(NP_001291287) c3 (zinc finger protein 42 homolog) c4(115633) c5(115634, 115635, 115636, 115637, 115638, 115639, 115640, 115641, 115642, 115643, 115644) c6( ) c7(es, T, d, Uc, iM, Db, wu, LX, eD, je, Hn, np, ci, wm, fr, lu, ae, eA, ad); #c1(ZFP57) c2(XP_006715150) c3(zinc finger protein 57 homolog isoform X2) c4( ) c5(288800, 288801, 288802, 288803, 288804, 288805) c6( ) c7(A, d, mh, lW, ml, eZ, aj, hH, fq, J); #c1(ZFP57) c2(XP_011512872) c3(zinc finger protein 57 homolog isoform X1) c4(304167, 304168, 304169, 304/70) c5(304/71, 304172, 304173, 304174, 304175, 304176, 304177, 304178, 304179, 304180, 304181, 304182, 304183, 304184, 304185, 304186, 304187, 304188, 304189, 304190, 304191, 304192, 304193, 304194, 304195, 304196, 304197, 304198, 304199, 304200, 304201, 304202, 304203, 304204, 304205, 304206, 304207, 304208, 304209, 304210, 304211, 304212, 304213, 304214, 304215, 304216, 304217, 304218, 304219, 304220, 304221, 304222, 304223, 304224, 304225, 304226, 304227, 304228, 304229, 304230, 304231, 304232, 304233, 304234, 304235, 304236, 304237, 304238, 304239, 304240, 304241, 304242, 304243, 304244, 304245, 304246, 304247) c6( ) c7(A, d, mh, lW, ml, eZ, aj, hH, fq, J); #c1(ZFP64) c2(NP_060667) c3(zinc finger protein 64 isoform a) c4(197073) c5(197074, 197075, 197076, 197077, 197078, 197079, 197080) c6( ) c7(c, aA); #c1(ZFP64) c2(NP_071371) c3(zinc finger protein 64 isoform b) c4(211223) c5(211224, 211225, 211226, 211227, 211228, 211229) c6( ) c7(c, aA); #c1(ZFP64) c2(NP_955458) c3(zinc finger protein 64 isoform c) c4(261778, 261779) c5(261780, 261781, 261782, 261783, 261784, 261785, 261786, 261787, 261788, 261789, 261790, 261791) c6( ) c7(c, aA); #c1(ZFP64) c2(NP_955459) c3(zinc finger protein 64 isoform d) c4(261792, 261793, 261794) c5(261795, 261796, 261797, 261798, 261799, 261800, 261801, 261802, 261803, 261804, 261805, 261806) c6( ) c7(c, aA); #c1(ZFP64) c2(XP_005260507) c3(zinc finger protein 64 isoform X3) c4(276572) c5(276573, 276574, 276575, 276576, 276577, 276578, 276579, 276580, 276581, 276582, 276583, 276584) c6( ) c7(c, aA); #c1(ZFP64) c2(XP_006723885) c3(zinc finger protein 64 isoform X2) c4(295417, 295418) c5(295419, 295420, 295421, 295422, 295423, 295424, 295425, 295426, 295427, 295428, 295429, 295430) c6( ) c7(c, aA); #c1(ZFP82) c2(NP_597723) c3(zinc finger protein 82 homolog) c4(235112, 235113) c5(235114, 235115, 235116, 235117, 235118, 235119) c6( ) c7(aF, aA); #c1(ZFP82) c2(XP_005258867) c3(zinc finger protein 82 homolog isoform X1) c4( ) c5(275838, 275839, 275840, 275841, 275842, 275843) c6( ) c7(aF, aA); #c1(ZFP91) c2(NP_444251) c3(E3 ubiquitin-protein ligase ZFP91 isoform 1) c4(230096, 230097) c5(230098, 230099, 230100, 230101, 230102, 230103, 230104, 230105, 230106, 230107, 230108, 230109, 230110) c6( ) c7(bt); #c1(ZFPM1) c2(NP_722520) c3(zinc finger protein ZFPM1) c4(246786) c5(246787, 246788, 246789, 246790, 246791, 246792, 246793) c6( ) c7(BU, oW, bzB, at, pg, dL, aGd, DU); #c1(ZFPM1) c2(XP_011521214) c3(zinc finger protein ZFPM1 isoform X1) c4( ) c5(316141) c6( ) c7(BU, oW, bzB, at, pg, dL, aGd, DU); #c1(ZFPM1) c2(XP_011521219) c3(zinc finger protein ZFPM1 isoform X6) c4( ) c5(316142) c6( ) c7(BU, oW, bzB, at, pg, dL, aGd, DU); #c1(ZFPM2) c2(NP_036214) c3(zinc finger protein ZFPM2) c4(172410) c5(172411, 172412, 172413, 172414, 172415, 172416, 172417) c6( ) c7(Mg, aLi, aeT, MS, vJ, acl, wk, cVz, bs, adW, UD, cm, aLn, bw, cVA, bD, t, er, cd, br); #c1(ZFPM2) c2(XP_011515249) c3(zinc finger protein ZFPM2 isoform X2) c4( ) c5(308064) c6( ) c7(Mg, aLi, aeT, MS, vJ, acl, wk, cVz, bs, adW, UD, cm, aLn, bw, cVA, bD, t, er, cd, br); #c1(ZFPM2) c2(XP_011515250) c3(zinc finger protein ZFPM2 isoform X3) c4(308065, 308066) c5(308067, 308068, 308069, 308070, 308071, 308072, 308073, 308074, 308075) c6( ) c7(Mg, aLi, aeT, MS, vJ, acl, wk, cVz, bs, adW, UD, cm, aLn, bw, cVA, bD, t, er, cd, br); #c1(ZFPM2) c2(XP_011515251) c3(zinc finger protein ZFPM2 isoform X4) c4( ) c5(308076, 308077, 308078, 308079, 308080, 308081, 308082) c6( ) c7(Mg, aLi, aeT, MS, vJ, acl, wk, cVz, bs, adW, UD, cm, aLn, bw, cVA, bD, t, er, cd, br); #c1(ZFR) c2(NP_057191) c3(zinc finger RNA-binding protein) c4(189383, 189384, 189385) c5(189386, 189387, 189388, 189389, 189390, 189391, 189392, 189393, 189394, 189395, 189396, 189397, 189398, 189399, 189400, 189401, 189402, 189403, 189404) c6( ) c7(sp); #c1(ZFR2) c2(NP_001139112) c3(zinc finger RNA-binding protein 2 isoform 2) c4(58692) c5(58693, 58694, 58695, 58696, 58697, 58698) c6( ) c7(cz); #c1(ZFR2) c2(NP_055989) c3(zinc finger RNA-binding protein 2 isoform 1) c4(184960, 184961) c5(184962, 184963, 184964, 184965, 184966, 184967) c6( ) c7(cz); #c1(ZFR2) c2(XP_011526135) c3(zinc finger RNA-binding protein 2 isoform X5) c4( ) c5(320401, 320402, 320403, 320404, 320405, 320406) c6( ) c7(cz); #c1(ZFX) c2(NP_001171557) c3(zinc finger X-chromosomal protein isoform 2) c4( ) c5(70577, 70578, 70579, 70580, 70581) c6( ) c7(T, eM, vJ, aW, aT, aA, Ej, Fb, af, eA, ae, B, ad); #c1(ZFX) c2(NP_001171566) c3(zinc finger X-chromosomal protein isoform 3) c4(70609) c5(70610, 70611, 70612, 70613, 70614, 70615, 70616, 70617, 70618, 70619, 70620, 70621, 70622, 70623, 70624, 70625, 70626, 70627, 70628, 70629, 70630, 70631) c6( ) c7(T, eM, vJ, aW, aT, aA, Ej, Fb, aF, eA, ae, B, ad); #c1(ZFX) c2(XP_005274649) c3(zinc finger X-chromosomal protein isoform X1) c4(285269, 285270, 285271, 285272) c5(285273, 285274, 285275, 285276, 285277, 285278, 285279, 285280, 285281, 285282, 285283, 285284, 285285, 285286, 285287, 285288, 285289, 285290, 285291, 285292, 285293) c6( ) c7(T, eM, vJ, aW, aT, aA, Ej, Fb, aF, eA, ae, B, ad); #c1(ZFX) c2(XP_006724578) c3 (zinc finger X-chromosomal protein isoform X3) c4(296087) c5(296088, 296089, 296090, 296091, 296092, 296093) c6( ) c7(T, eM, vJ, aW, aT, aA, Ej, Fb, aF, eA, ae, B, ad); #c1(ZFX) c2(XP_011543880) c3(zinc finger X-chromosomal protein isoform X2) c4(343051, 343052) c5(343053, 343054, 343055, 343056, 343057, 343058) c6( ) c7(T, eM, vJ, aW, aT, aA, Ej, Fb, aF, eA, ae, B, ad); #c1(ZFX) c2(XP_011543885) c3(zinc finger X-chromosomal protein isoform X4) c4(343059) c5(343060, 343061) c6( ) c7(T, eM, vJ, aW, aT, aA, Ej, Fb, aF, eA, ae, B, ad); #c1(ZFY) c2(NP_001138747) c3(zinc finger Y-chromosomal protein isoform 2) c4(57519) c5( ) c6( ) c7(bfh, aeT, clZ, vJ, bff, UD, Pp, Fb, g, f, Df); #c1(ZFY) c2(NP_001138748) c3(zinc finger Y-chromosomal protein isoform 3) c4(57520) c5(57521, 57522, 57523, 57524, 57525, 57526) c6( ) c7(bfh, aeT, clZ, vJ, bff, UD, Pp, Fb, g, f, Df); #c1(ZFY) c2(XP_006724936) c3(zinc finger Y-chromosomal protein isoform X1) c4(296494) c5(296495, 296496, 296497, 296498, 296499, 296500, 296501, 296502, 296503, 296504, 296505, 296506, 296507) c6( ) c7(bfh, aeT, clZ, vJ, bff, UD, Pp, Fb, g, f, Df); #c1(ZFY) c2(XP_011529777) c3(zinc finger Y-chromosomal protein isoform X3) c4(325565) c5(325566, 325567, 325568, 325569, 325570, 325571, 325572, 325573, 325574, 325575, 325576, 325577, 325578) c6( ) c7(bfh, aeT, clZ, vJ, bff, UD, Pp, Fb, g, f, Df); #c1(ZFYVE19) c2(NP_001070736) c3(abscission/NoCut checkpoint regulator isoform 1) c4(34835) c5(34836, 34837, 34838, 34839, 34840, 34841) c6( ) c7(ck); #c1(ZFYVE19) c2(NP_001245349) c3(abscission/NoCut checkpoint regulator isoform 2) c4(93209) c5(93210, 93211, 93212, 93213, 93214, 93215) c6( ) c7(ck); #c1(ZFYVE19) c2(NP_001245350) c3(abscission/NoCut checkpoint regulator isoform 4) c4( ) c5(93216, 93217, 93218, 93219, 93220) c6( ) c7(ck); #c1(ZFYVE19) c2(NP_116239) c3(abscission/NoCut checkpoint regulator isoform 3) c4(225902, 225903) c5(225904, 225905, 225906, 225907, 225908, 225909, 225910) c6( ) c7(ck); #c1(ZFYVE21) c2(NP_001185882) c3(zinc finger FYVE domain-containing protein 21 isoform 1) c4(76635) c5(76636, 76637, 76638, 76639, 76640, 76641) c6( ) c7(hk, Y); #c1(ZFYVE21) c2(NP_076976) c3(zinc finger FYVE domain-containing protein 21 isoform 2) c4(214550) c5(214551, 214552, 214553, 214554, 214555, 214556) c6( ) c7(hk, Y); #c1(ZFYVE26) c2(NP_056161) c3(zinc finger FYVE domain-containing protein 26) c4(186040, 186041) c5(186042, 186043, 186044, 186045, 186046, 186047, 186048, 186049, 186050, 186051, 186052, 186053) c6( ) c7(ky, bcS, aV, aW, Kk, clB, ha, tx, aT, nw, bk, nA, Qn); #c1(ZFYVE27) c2(NP_001002261) c3(protrudin isoform a) c4(19477, 19478) c5(19479, 19480, 19481, 19482, 19483, 19484, 19485, 19486, 19487, 19488, 19489, 19490) c6( ) c7(aV, cVB); #c1(ZFYVE27) c2(NP_001002262) c3(protrudin isoform c) c4(19491, 19492) c5(19493, 19494, 19495, 19496, 19497, 19498, 19499, 19500, 19501, 19502, 19503, 19504) c6( ) c7(aV, cVB); #c1(ZFYVE27) c2(NP_001167590) c3(protrudin isoform d) c4(69353) c5(69354, 69355, 69356, 69357, 69358, 69359) c6( ) c7(aV, cVB); #c1(ZFYVE27) c2(NP_001167591) c3(protrudin isoform e) c4(69360) c5(69361, 69362, 69363, 69364, 69365, 69366) c6( ) c7(aV, cVB); #c1(ZFYVE27) c2(NP_001167592) c3(protrudin isoform f) c4(69367) c5(69368, 69369, 69370, 69371, 69372) c6( ) c7(aV, cVB); #c1(ZFYVE27) c2(NP_001167593) c3(protrudin isoform g) c4(69373) c5(69374, 69375, 69376, 69377, 69378, 69379) c6( ) c7(aV, cVB); #c1(ZFYVE27) c2(NP_653189) c3(protrudin isoform b) c4(238993) c5(238994, 238995, 238996, 238997, 238998, 238999) c6( ) c7(aV, cVB); #c1(ZFYVE27) c2(XP_005269562) c3(protrudin isoform X4) c4( ) c5(282647) c6( ) c7(aV, cVB); #c1(ZFYVE28) c2(NP_001166 (27) c3(lateral signaling target protein 2 homolog isoform 3) c4(68255) c5(68256, 68257, 68258, 68259, 68260, 68261) c6( ) c7(Ea); #c1(ZFYVE28) c2(NP_001166128) c3(lateral signaling target protein 2 homolog isoform 4) c4(68262) c5(68263, 68264, 68265, 68266, 68267, 68268) c6( ) c7(Ea); #c1(ZFYVE28) c2(NP_001166129) c3(lateral signaling target protein 2 homolog isoform 5) c4(68269) c5(68270, 68271, 68272, 68273, 68274, 68275) c6( ) c7(Ea); #c1(ZFYVE28) c2(NP_001166130) c3(lateral signaling target protein 2 homolog isoform 1) c4( ) c5(68276, 68277, 68278, 68279, 68280) c6( ) c7(Ea); #c1(ZFYVE28) c2(NP_001166131) c3(lateral signaling target protein 2 homolog isoform 6) c4( ) c5(68281, 68282, 68283, 68284, 68285) c6( ) c7(Ea); #c1(ZFYVE28) c2(NP_066023) c3(lateral signaling target protein 2 homolog isoform 2) c4(206787, 206788) c5(206789, 206790, 206791, 206792, 206793, 206794, 206795) c6( ) c7(Ea); #c1(ZFYVE28) c2(XP_006713963) c3(lateral signaling target protein 2 homolog isoform X1) c4(287434) c5( ) c6( ) c7(Ea); #c1(ZFYVE28) c2(XP_006713967) c3(lateral signaling target protein 2 homolog isoform X8) c4( ) c5(287435) c6( ) c7(Ea); #c1(ZFYVE9) c2(XP_011540739) c3(zinc finger FYVE domain-containing protein 9 isoform X1) c4(339299, 339300) c5(339301, 339302, 339303, 339304, 339305, 339306, 339307, 339308, 339309, 339310, 339311, 339312, 339313, 339314, 339315, 339316) c6( ) c7(T, jz, fM, y, wO, ds, tT, anV, aW, aT, m, akr, t, ik, q, ad, aa); #c1(ZG16B) c2(NP_660295) c3(zymogen granule protein 16 homolog B precursor) c4(240770) c5(240771, 240772, 240773, 240774, 240775, 240776, 240777, 240778) c6(240779, 240780) c7(T, aC, aE); #c1(ZGLP1) c2(NP_001096637) c3(GATA-type zinc finger protein 1) c4(41256) c5(41257, 41258, 41259, 41260, 41261, 41262) c6( ) c7(bL, mj, A, dA, e, al, jy, blg, dG, j, aj, v, ra, J, hm); #c1(ZGPAT) c2(NP_001076582) c3(zinc finger CCCH-type with G patch domain-containing protein isoform c) c4(37501) c5(37502, 37503, 37504, 37505, 37506, 37507, 37508, 37509, 37510, 37511, 37512, 37513, 37514, 37515) c6( ) c7(bL, A, kj, yw, aE, cz, pz, fe, aj, zx, K, j, eA, y, ae, B, J, ad); #c1(ZGPAT) c2(NP_001182583) c3(zinc finger CCCH-type with G patch domain-containing protein isoform d) c4(75095) c5(75096, 75097, 75098, 75099, 75100, 75101, 75102, 75103, 75104, 75105, 75106, 75107) c6( ) c7(bL, A, kj, yw, aE, cz, pz, fe, aj, zx, K, j, eA, y, ae, B, J, ad); #c1(ZGPAT) c2(NP_115916) c3(zinc finger CCCH-type with G patch domain-containing protein isoform a) c4(224667, 224668) c5(224669, 224670, 224671, 224672, 224673, 224674) c6( ) c7(bL, A, kj, yw, aE, cz, pz, fe, aj, zx, K, j, eA, y, ae, B, J, ad); #c1(ZHX1) c2(NP_009153) c3(zinc fingers and homeoboxes protein 1) c4(171162) c5(171163, 171164, 171165, 171166, 171167, 171168, 171169, 171170, 171171, 171172, 171173, 171174, 171175, 171176, 171177) c6( ) c7(aF, aA); #c1(ZHX2) c2(XP_011515234) c3(zinc fingers and homeoboxes protein 2 isoform X1) c4(308048, 308049) c5(308050, 308051, 308052, 308053, 308054, 308055, 308056, 308057, 308058, 308059, 308060, 308061, 308062) c6( ) c7(T, yS, yk, eX, bO, aA, bB, fd, kv, ci, Qp, q, ad, eA, zn, dP, afr, hd, aE, Rq, nv, pX, nl, gd, aT, cyn, eV, K, lQ, ae, cp, eG, Uf, OS, BO, il, aW, Y, eH, nh, r, aq, kp, aF, g, cl, as, eF, iR, bz, Qt, iP, Hp, ck, nM, Dy, eM, nB, dB, bE, iT, Ew, aaG, Aw, adg, bt, B, fW); #c1(ZIC1) c2(NP_003403) c3(zinc finger protein ZIC 1) c4(138187) c5(138188, 138189, 138190, 138191, 138192, 138193, 138194) c6( ) c7(T, YU, aA, bB, Xr, V, q, aR, ft, hd, MG, jf, Ng, nO, r, atf, aF, bz, nM, nB, dB, lp, bE, au, gX, Km, ZW, B); #c1(ZIC2) c2(NP_009060) c3 (zinc finger protein ZIC 2) c4(170541, 170542) c5(170543, 170544, 170545, 170546, 170547, 170548, 170549, 170550) c6( ) c7(jf, cZ, ft, bPl, bPt, lp, if, FB, asl, ff, Be, r, Xr, en, cl, qN, ZW, q, aR, ad, MG); #c1(ZIC2) c2(XP_011519412) c3(zinc finger protein ZIC 2 isoform X1) c4(314078) c5(314079, 314080, 314081, 314082, 314083, 314084, 314085, 314086, 314087, 314088, 314089) c6( ) c7(jf, cZ, ft, bPl, bPt, lp, if, FB, asl, ff, Be, r, Xr, en, cl, qN, ZW, q, aR, ad, MG); #c1(ZIC3) c2(NP_003404) c3(zinc finger protein ZIC 3) c4(138195, 138196) c5(138197, 138198, 138199, 138200, 138201, 138202, 138203, 138204, 138205, 138206, 138207, 138208, 138209) c6( ) c7(md, gV, nD, cVC, hG, lp, bgH, ckm, aMS, Az, byd, gv, brc, j, bD, BV, fq, Gc); #c1(ZIC4) c2(NP_001161850) c3(zinc finger protein ZIC 4 isoform 1 precursor) c4(66164) c5(66165, 66166, 66167, 66168, 66169, 66170, 66171, 66172, 66173, 66174) c6( ) c7(atf, ft, Ng, Xr, MG); #c1(ZIC4) c2(NP_001161851) c3(zinc finger protein ZIC 4 isoform 2) c4(66175, 66/76, 66177, 66178, 66179) c5(66180, 66181, 66182, 66183, 66184, 66185, 66186, 66187, 66188) c6( ) c7(atf, ft, Ng, Xr, MG); #c1(ZIC4) c2(XP_011511533) c3(zinc finger protein ZIC 4 isoform X1) c4( ) c5(302223, 302224, 302225, 302226, 302227, 302228, 302229, 302230, 302231, 302232, 302233, 302234, 302235, 302236, 302237, 302238, 302239, 302240, 302241, 302242, 302243, 302244, 302245, 302246, 302247, 302248) c6( ) c7(atf, ft, Ng, Xr, MG); #c1(ZIC5) c2(NP_149123) c3(zinc finger protein ZIC 5) c4(227302, 227303) c5(227304, 227305, 227306, 227307, 227308, 227309, 227310, 227311, 227312, 227313, 227314, 227315, 227316, 227317, 227318) c6( ) c7(Xr, FB, Az, ZW); #c1(ZIK1) c2(NP_001010879) c3(zinc finger protein interacting with ribonucleoprotein K) c4(23962, 23963) c5(23964, 23965, 23966, 23967, 23968, 23969, 23970, 23971, 23972, 23973, 23974) c6( ) c7(T); #c1(ZIK1) c2(XP_011525062) c3(zinc finger protein interacting with ribonucleoprotein K isoform X1) c4(319524) c5(319525, 319526, 319527, 319528, 319529, 319530) c6( ) c7(T); #c1(ZIK1) c2(XP_011525064) c3(zinc finger protein interacting with ribonucleoprotein K isoform X3) c4( ) c5(319531, 319532, 319533, 319534, 319535) c6( ) c7(T); #c1(ZIM2) c2(NP_001139799) c3(zinc finger imprinted 2) c4(59958) c5(59959, 59960, 59961, 59962, 59963, 59964, 59965, 59966, 59967, 59968, 59969, 59970, 59971, 59972, 59973, 59974, 59975, 59976, 59977, 59978, 59979, 59980, 59981, 59982, 59983, 59984) c6( ) c7(qK); #c1(ZKSCAN1) c2(NP_001273983) c3(zinc finger protein with KRAB and SCAN domains 1 isoform b) c4( ) c5(105952, 105953, 105954, 105955, 105956, 105957) c6( ) c7(cOR, aF, ml, ei, aA); #c1(ZKSCAN1) c2(NP_001273984) c3(zinc finger protein with KRAB and SCAN domains 1 isoform c) c4( ) c5(105958, 105959, 105960, 105961, 105962, 105963) c6( ) c7(cOR, aF, ml, ei, aA); #c1(ZKSCAN1) c2(NP_003430) c3(zinc finger protein with KRAB and SCAN domains 1 isoform a) c4(138367, 138368, 138369, 138370) c5(138371, 138372, 138373, 138374, 138375, 138376, 138377) c6( ) c7(cOR, aF, ml, ei, aA); #c1(ZKSCAN1) c2(XP_005250622) c3(zinc finger protein with KRAB and SCAN domains 1 isoform X2) c4( ) c5(270462, 270463, 270464, 270465, 270466, 270467) c6( ) c7(cOR, aF, ml, ei, aA); #c1(ZKSCAN3) c2(NP_001229824) c3(zinc finger protein with KRAB and SCAN domains 3 isoform 2) c4( ) c5(85343, 85344, 85345, 85346, 85347, 85348) c6( ) c7(T, bE, r, bB, fd, Kj, K, ae, q, ad); #c1(ZKSCAN3) c2(NP_077819) c3(zinc finger protein with KRAB and SCAN domains 3 isoform 1) c4(215400, 215401) c5(215402, 215403, 215404, 215405, 215406, 215407, 215408, 215409, 215410, 215411, 215412, 215413, 215414, 215415, 215416, 215417) c6( ) c7(T, bE, r, bB, fd, Kj, K, ae, q, ad); #c1(ZKSCAN3) c2(XP_005249480) c3(zinc finger protein with KRAB and SCAN domains 3 isoform X4) c4( ) c5(269497) c6( ) c7(T, bE, r, bB, fd, Kj, K, ae, q, ad); #c1(ZKSCAN3) c2(XP_006715279) c3(zinc finger protein with KRAB and SCAN domains 3 isoform X3) c4(289478, 289479) c5( ) c6( ) c7(T, bE, r, bB, fd, Kj, K, ae, q, ad); #c1(ZKSCAN7) c2(NP_001275520) c3(zinc finger protein with KRAB and SCAN domains 7 isoform 2) c4( ) c5(106805, 106806, 106807, 106808, 106809, 106810, 106811, 106812, 106813, 106814) c6( ) c7(T, uT, vA, aj, Lc, cm, J, cd, op); #c1(ZKSCAN7) c2(NP_061121) c3(zinc finger protein with KRAB and SCAN domains 7 isoform 1) c4(199232, 199233) c5(199234, 199235, 199236, 199237, 199238, 199239, 199240, 199241, 199242, 199243, 199244) c6( ) c7(T, uT, vA, aj, Lc, cm, J, cd, op); #c1(ZKSCAN7) c2(XP_011547035) c3(zinc finger protein with KRAB and SCAN domains 7 isoform X1) c4(345862) c5( ) c6( ) c7(T, uT, vA, aj, Lc, cm, J, cd, op); #c1(ZKSCAN7) c2(XP_011547042) c3(zinc finger protein with KRAB and SCAN domains 7 isoform X7) c4( ) c5(345863, 345864, 345865, 345866, 345867, 345868) c6( ) c7(T, uT, vA, aj, Lc, cm, J, cd, op); #c1(ZMAT3) c2(XP_005247763) c3(zinc finger matrin-type protein 3 isoform X2) c4(267969) c5(267970, 267971, 267972, 267973, 267974, 267975, 267976, 267977, 267978, 267979, 267980, 267981) c6( ) c7(T, bE, Qu, ff, ht, HC, cj, bB, en, hu, cm, it); #c1(ZMAT3) c2(XP_011511375) c3(zinc finger matrin-type protein 3 isoform X1) c4(301949, 301950) c5(301951, 301952, 301953, 301954, 301955, 301956) c6( ) c7(T, bE, Qu, ff, ht, HC, cj, bB, en, hu, cm, it); #c1(ZMAT4) c2(NP_001129203) c3(zinc finger matrin-type protein 4 isoform b) c4( ) c5(51169, 51170, 51171, 51172, 51173) c6( ) c7(Bd, bK, Be, an); #c1(ZMAT4) c2(NP_078921) c3(zinc finger matrin-type protein 4 isoform a) c4( ) c5(216124, 216125, 216126, 216127, 216128, 216129, 216130, 216131, 216132, 216133) c6( ) c7(Bd, bK, Be, an); #c1(ZMAT4) c2(XP_011542945) c3(zinc finger matrin-type protein 4 isoform X1) c4(341651, 341652) c5(341653, 341654) c6( ) c7(Bd, bK, Be, an); #c1(ZMAT4) c2(XP_011542947) c3(zinc finger matrin-type protein 4 isoform X3) c4(341655) c5(341656) c6( ) c7(Bd, bK, Be, an); #c1(ZMIZ1) c2(NP_065071) c3(zinc finger MIZ domain-containing protein 1) c4(203618, 203619, 203620) c5(203621, 203622, 203623, 203624, 203625, 203626, 203627, 203628, 203629, 203630, 203631, 203632) c6( ) c7(bL, ck, T, iV, A, aW, hk, ff, Qo, pQ, cr, aT, bB, fe, bK, k, hp, qj, en, V, B); #c1(ZMIZ1) c2(XP_006717987) c3(zinc finger MIZ domain-containing protein 1 isoform X1) c4(291412) c5( ) c6( ) c7(bL, ck, T, iV, A, aW, hk, ff, Qo, pQ, cr, aT, bB, fe, bk, k, hp, qj, en, V, B); #c1(ZMIZ1) c2(XP_006717988) c3(zinc finger MIZ domain-containing protein 1 isoform X2) c4(291413) c5(291414, 291415, 291416, 291417, 291418, 291419, 291420, 291421, 291422, 291423, 291424, 291425) c6( ) c7(bL, ck, T, iV, A, aW, hk, ff, Qo, pQ, cr, aT, bB, fe, bK, k, hp, qj, en, V, B); #c1(ZMIZ1) c2(XP_011538282) c3(zinc finger MIZ domain-containing protein 1 isoform X4) c4( ) c5(335947, 335948, 335949, 335950, 335951, 335952) c6( ) c7(bL, ck, T, iV, A, aW, hk, ff, Qo, pQ, cr, aT, bB, fe, bk, k, hp, qj, en, V, B); #c1(ZMYM2) c2(XP_011533519) c3(zinc finger MYM-type protein 2 isoform X3) c4( ) c5(329622, 329623, 329624, 329625) c6( ) c7(btw, et, fd, aA, nf, n, dz, aq, aF, m, ci, pg, iR, t, kS, B, hX); #c1(ZMYM2) c2(XP_011533523) c3(zinc finger MYM-type protein 2 isoform X1) c4(329626, 329627, 329628) c5(329629, 329630, 329631, 329632, 329633, 329634, 329635, 329636, 329637, 329638, 329639, 329640, 329641, 329642, 329643, 329644) c6( ) c7(btw, et, fd, aA, nf, n, dz, aq, aF, m, ci, pg, iR, t, kS, B, hX); #c1(ZMYM2) c2(XP_011533524) c3(zinc finger MYM-type protein 2 isoform X4) c4( ) c5(329645, 329646, 329647, 329648, 329649, 329650, 329651) c6( ) c7(btw, et, fd, aA, nf, n, dz, aq, aF, m, ci, pg, iR, t, kS, B, hX); #c1(ZMYM3) c2(NP_001164633) c3(zinc finger MYM-type protein 3 isoform 2) c4(67092, 67093) c5(67094, 67095, 67096, 67097, 67098, 67099) c6( ) c7(nf, jf, cj); #c1(ZMYM3) c2(NP_001164634) c3(zinc finger MYM-type protein 3 isoform 3) c4(67100, 67101) c5(67102, 67103, 67104, 67105, 67106, 67107, 67108, 67109, 67110, 67111, 67112, 67113, 67114, 67115, 67116, 67117, 67118, 67119, 67120) c6( ) c7(nf, jf, cj); #c1(ZMYM3) c2(NP_005087) c3(zinc finger MYM-type protein 3 isoform 1) c4(152765, 152766, 152767) c5(152768, 152769, 152770, 152771, 152772, 152773, 152774, 152775, 152776, 152777, 152778, 152779, 152780, 152781, 152782, 152783, 152784, 152785, 152786, 152787, 152788, 152789, 152790) c6( ) c7(nf, jf, cj); #c1(ZMYM4) c2(XP_011540726) c3(zinc finger MYM-type protein 4 isoform X5) c4(339288, 339289) c5( ) c6( ) c7(g, ff, en); #c1(ZMYM5) c2(NP_001034738) c3(zinc finger MYM-type protein 5 isoform 2) c4(32112) c5(32113, 32114, 32115, 32116, 32117, 32118) c6( ) c7(kl); #c1(ZMYM5) c2(NP_001034739) c3(zinc finger MYM-type protein 5 isoform 1) c4(32119) c5(32120, 32121, 32122, 32123, 32124, 32125) c6( ) c7(kl); #c1(ZMYM5) c2(NP_001136156) c3(zinc finger MYM-type protein 5 isoform 3) c4( ) c5(54451, 54452, 54453, 54454, 54455) c6( ) c7(kl); #c1(ZMYM5) c2(XP_011533611) c3(zinc finger MYM-type protein 5 isoform X2) c4(329721) c5(329722) c6( ) c7(kl); #c1(ZMYND10) c2(NP_056980) c3(zinc finger MYND domain-containing protein 10) c4(188319) c5(188320, 188321, 188322, 188323, 188324, 188325) c6( ) c7(T, if, ci, eA, br, av, jz, ds, io, dL, Nj, K, cVD, eL, MG, Pf, ff, lo, agF, bxx, as, g, en, cl, t, bz, cm, bw, eM, aiM, hL, m, it, B); #c1(ZMYND11) c2(NP_001189394) c3(zinc finger MYND domain-containing protein 11 isoform d) c4(79337) c5(79338, 79339, 79340, 79341, 79342, 79343) c6( ) c7(bN, aW, aw, nA, aT); #c1(ZMYND11) c2(NP_001189397) c3(zinc finger MYND domain-containing protein 11 isoform g) c4(79344, 79345) c5(79346, 79347, 79348, 79349, 79350, 79351, 79352, 79353, 79354, 79355, 79356, 79357) c6( ) c7(bN, aW, aw, nA, aT); #c1(ZMYND11) c2(NP_997644) c3(zinc finger MYND domain-containing protein 11 isoform b) c4(265657) c5(265658, 265659, 265660, 265661, 265662, 265663) c6( ) c7(bN, aW, aw, nA, aT); #c1(ZMYND11) c2(XP_005252416) c3(zinc finger MYND domain-containing protein 11 isoform X1) c4(271725, 271726) c5(271727, 271728, 271729, 271730, 271731, 271732, 271733, 271734, 271735, 271736, 271737, 271738, 271739, 27/740) c6( ) c7(bN, aW, aw, nA, aT); #c1(ZMYND11) c2(XP_006717439) c3(zinc finger MYND domain-containing protein 11 isoform X2) c4(291093, 291094, 291095) c5(291096, 291097, 291098, 291099, 291100, 291101, 291102, 29/103, 291104, 291105, 291106, 291107, 291108, 291109, 291110, 291111, 29/112, 291113) c6( ) c7(bN, aW, aw, nA, aT); #c1(ZMYND11) c2(XP_011517603) c3(zinc finger MYND domain-containing protein 11 isoform X5) c4(311373) c5(311374, 311375, 311376, 311377, 311378, 311379, 311380, 311381, 311382, 311383, 311384, 311385, 311386, 311387, 311388) c6( ) c7(bN, aW, aw, nA, aT); #c1(ZMYND11) c2(XP_011517604) c3(zinc finger MYND domain-containing protein 11 isoform X3) c4(311389) c5(311390, 311391, 311392, 311393, 311394, 311395) c6( ) c7(bN, aW, aw, nA, aT); #c1(ZMYND8) c2(NP_001268698) c3(protein kinase C-binding protein 1 isoform d) c4( ) c5(100147, 100148, 100149, 100150, 100151, 100152, 100153, 100154, 100155, 100156, 100157) c6( ) c7(bL, ae, ad); #c1(ZMYND8) c2(NP_001268701) c3(protein kinase C-binding protein 1 isoform f) c4(100158, 100159) c5(100160, 100161, 100162, 100163, 100164, 100165) c6( ) c7(bL, ae, ad); #c1(ZMYND8) c2(NP_001268703) c3(protein kinase C-binding protein 1 isoform h) c4( ) c5(100166, 100167, 100168, 100169, 100170, 100171, 100172, 100173, 100174, 100175, 100176) c6( ) c7(bL, ae, ad); #c1(ZMYND8) c2(NP_001268704) c3(protein kinase C-binding protein 1 isoform i) c4(100177) c5(100178, 100179, 100180, 100181, 100182, 100183) c6( ) c7(bL, ae, ad); #c1(ZMYND8) c2(NP_001268706) c3(protein kinase C-binding protein 1 isoform k) c4(100184) c5(100185, 100186, 100187, 100188, 100189, 100190) c6( ) c7(bL, ae, ad); #c1(ZMYND8) c2(NP_001268710) c3(protein kinase C-binding protein 1 isoform o) c4(100191) c5( ) c6( ) c7(bL, ae, ad); #c1(ZMYND8) c2(NP_001268711) c3(protein kinase C-binding protein 1 isoform p) c4(100192) c5(100193, 100194, 100195, 100196, 100197, 100198) c6( ) c7(bL, ae, ad); #c1(ZMYND8) c2(NP_001268712) c3(protein kinase C-binding protein 1 isoform q) c4( ) c5(100199, 100200, 100201, 100202, 100203, 100204) c6( ) c7(bL, ae, ad); #c1(ZMYND8) c2(NP_001268713) c3(protein kinase C-binding protein 1 isoform r) c4(100205) c5(100206, 100207, 100208, 100209, 100210, 100211) c6( ) c7(bL, ae, ad); #c1(ZMYND8) c2(NP_036540) c3(protein kinase C-binding protein 1 isoform b) c4(174293) c5(174294, 174295, 174296, 174297, 174298, 174299) c6( ) c7(bL, ae, ad); #c1(ZMYND8) c2(NP_898868) c3(protein kinase C-binding protein 1 isoform a) c4(257790, 257791, 257792) c5(257793, 257794, 257795, 257796, 257797, 257798, 257799) c6( ) c7(bL, ae, ad); #c1(ZMYND8) c2(NP_898869) c3(protein kinase C-binding protein 1 isoform c) c4(257800) c5(257801, 257802, 257803, 257804, 257805, 257806) c6( ) c7(bL, ae, ad); #c1(ZMYND8) c2(XP_005260413) c3 (protein kinase C-binding protein 1 isoform X2) c4(276531) c5(276532, 276533, 276534, 276535, 276536, 276537, 276538) c6( ) c7(bL, ae, ad); #c1(ZMYND8) c2(XP_005260423) c3(protein kinase C-binding protein 1 isoform X11) c4(276539) c5( ) c6( ) c7(bL, ae, ad); #c1(ZMYND8) c2(XP_006723826) c3(protein kinase C-binding protein 1 isoform X8) c4( ) c5(295407) c6( ) c7(bL, ae, ad); #c1(ZMYND8) c2(XP_011527057) c3 (protein kinase C-binding protein 1 isoform X12) c4( ) c5(321500) c6( ) c7(bL, ae, ad); #c1(ZNF10) c2(NP_056209) c3(zinc finger protein 10) c4(186340, 186341, 186342, 186343, 186344, 186345) c5(186346, 186347, 186348, 186349, 186350, 186351, 186352, 186353, 186354, 186355, 186356, 186357, 186358, 186359, 186360, 186361, 186362, 186363, 186364, 186365, 186366, 186367, 186368, 186369, 186370, 186371, 186372, 186373, 186374, 186375, 186376, 186377) c6(186378, 186379) c7(hf); #c1(ZNF106) c2(NP_001271236) c3(zinc finger protein 106 isoform 3) c4(103686) c5(103687, 103688, 103689, 103690, 103691, 103692) c6( ) c7(fq); #c1(ZNF106) c2(NP_071918) c3(zinc finger protein 106 isoform 1) c4(212290) c5(212291, 212292, 212293, 212294, 212295, 212296, 212297) c6( ) c7(fq); #c1(ZNF107) c2(NP_001013768) c3(zinc finger protein 107 isoform a) c4(25759, 25760) c5(25761, 25762, 25763, 25764, 25765, 25766, 25767, 25768, 25769, 25770) c6( ) c7(h); #c1(ZNF107) c2(NP_001269288) c3(zinc finger protein 107 isoform b) c4(101034) c5(101035, 101036, 101037, 101038, 101039, 101040, 101041, 101042, 101043, 101044, 101045, 101046, 101047, 101048, 101049, 101050, 101051, 101052, 101053) c6( ) c7(h); #c1(ZNF107) c2(NP_001269289) c3(zinc finger protein 107 isoform c) c4( ) c5(101054) c6( ) c7(h); #c1(ZNF112) c2(NP_001076804) c3(zinc finger protein 112 isoform 1) c4(37547, 37548, 37549, 37550, 37551) c5(37552, 37553, 37554, 37555, 37556, 37557, 37558, 37559, 37560, 37561, 37562, 37563, 37564, 37565, 37566, 37567, 37568, 37569, 37570, 37571, 37572, 37573, 37574, 37575, 37576, 37577, 37578, 37579, 37580, 37581, 37582) c6( ) c7(eZ); #c1(ZNF121) c2(XP_011526569) c3(zinc finger protein 121 isoform X2) c4( ) c5(320873) c6( ) c7(Db, hd, nB); #c1(ZNF131) c2(NP_001284477) c3(zinc finger protein 131 isoform 1) c4(111845) c5(111846, 111847, 111848, 111849, 111850, 111851, 111852, 111853, 111854, 111855) c6( ) c7(aW, ey, aT); #c1(ZNF131) c2(NP_003423) c3(zinc finger protein 131 isoform 2) c4(138322, 138323, 138324, 138325) c5(138326, 138327, 138328, 138329, 138330, 138331, 138332, 138333, 138334, 138335, 138336, 138337, 138338, 138339, 138340, 138341, 138342, 138343, 138344, 138345, 138346, 138347, 138348, 138349, 138350, 138351, 138352, 138353, 138354, 138355, 138356, 138357) c6( ) c7(aW, ey, aT); #c1(ZNF131) c2(XP_011512426) c3(zinc finger protein 131 isoform X3) c4( ) c5(303127) c6( ) c7(aW, ey, aT); #c1(ZNF132) c2(NP_003424) c3(zinc finger protein 132) c4(138358, 138359) c5(138360, 138361, 138362, 138363, 138364, 138365, 138366) c6( ) c7(aw, eu, e, j, ac); #c1(ZNF132) c2(XP_005259267) c3(zinc finger protein 132 isoform X1) c4( ) c5(276022, 276023, 276024, 276025, 276026, 276027) c6( ) c7(aw, eu, e, j, ac); #c1(ZNF133) c2(NP_001269925) c3(zinc finger protein 133 isoform b) c4(103247, 103248) c5(103249, 103250, 103251, 103252, 103253, 103254, 103255) c6( ) c7(acw); #c1(ZNF133) c2(NP_001269930) c3(zinc finger protein 133 isoform c) c4( ) c5(103256, 103257, 103258, 103259, 103260, 103261, 103262, 103263, 103264, 103265) c6( ) c7(acw); #c1(ZNF133) c2(NP_001269931) c3(zinc finger protein 133 isoform d) c4(103266) c5(103267, 103268) c6( ) c7(acw); #c1(ZNF133) c2(NP_001269934) c3(zinc finger protein 133 isoform f) c4( ) c5(103269) c6( ) c7(acw); #c1(ZNF133) c2(NP_001269935) c3(zinc finger protein 133 isoform g) c4( ) c5(103270) c6( ) c7(acw); #c1(ZNF133) c2(XP_005260875) c3(zinc finger protein 133 isoform X4) c4(276944) c5(276945, 276946, 276947, 276948, 276949, 276950, 276951) c6( ) c7(acw); #c1(ZNF133) c2(XP_005260877) c3(zinc finger protein 133 isoform X1) c4( ) c5(276952, 276953, 276954, 276955, 276956, 276957, 276958, 276959) c6( ) c7(acw); #c1(ZNF133) c2(XP_006723680) c3(zinc finger protein 133 isoform X2) c4(295284) c5(295285, 295286, 295287, 295288, 295289) c6( ) c7(acw); #c1(ZNF133) c2(XP_006723681) c3(zinc finger protein 133 isoform X6) c4( ) c5(295290, 295291, 295292, 295293, 295294, 295295, 295296, 295297) c6( ) c7(acw); #c1(ZNF133) c2(XP_011527636) c3(zinc finger protein 133 isoform X3) c4(322549, 322550, 322551) c5(322552, 322553, 322554, 322555, 322556, 322557, 322558, 322559) c6( ) c7(acw); #c1(ZNF141) c2(NP_003432) c3(zinc finger protein 141) c4(138378, 138379) c5(138380, 138381, 138382, 138383, 138384, 138385, 138386, 138387, 138388, 138389, 138390, 138391, 138392) c6( ) c7(rR, eX, cVE, eV, zG, axC); #c1(ZNF141) c2(XP_011511864) c3(zinc finger protein 141 isoform X1) c4(302595) c5(302596, 302597, 302598, 302599, 302600) c6( ) c7(rR, eX, cVE, eV, zG, axC); #c1(ZNF143) c2(NP_001269585) c3(zinc finger protein 143 isoform 2) c4(102061, 102062, 102063) c5(102064, 102065, 102066, 102067, 102068) c6( ) c7(ae, T, alC, bE, ml, bB, cOR, bz, ad); #c1(ZNF143) c2(NP_001269586) c3(zinc finger protein 143 isoform 3) c4(102069) c5(102070, 102071, 102072, 102073, 102074, 102075) c6( ) c7(ae, T, alC, bE, ml, bB, cOR, bz, ad); #c1(ZNF143) c2(NP_003433) c3(zinc finger protein 143 isoform 1) c4(138393, 138394) c5(138395, 138396, 138397, 138398, 138399) c6( ) c7(ae, T, alC, bE, ml, bB, cOR, bz, ad); #c1(ZNF143) c2(XP_005253178) c3(zinc finger protein 143 isoform X2) c4(272246, 272247) c5(272248, 272249) c6( ) c7(ae, T, alC, bE, ml, bB, cOR, bz, ad); #c1(ZNF143) c2(XP_005253179) c3(zinc finger protein 143 isoform X3) c4( ) c5(272250) c6( ) c7(ae, T, alC, bE, ml, bB, cOR, bz, ad); #c1(ZNF143) c2(XP_005253183) c3(zinc finger protein 143 isoform X4) c4( ) c5(272251, 272252, 272253, 272254, 272255, 272256, 272257, 272258, 272259, 272260, 272261, 272262, 272263, 272264, 272265, 272266, 272267, 272268, 272269, 272270, 272271, 272272, 272273, 272274, 272275, 272276, 272277, 272278, 272279, 272280) c6( ) c7(ae, T, alC, bE, ml, bB, cOR, bz, ad); #c1(ZNF146) c2(XP_005259271) c3(zinc finger protein OZF isoform X1) c4(276028) c5(276029, 276030, 276031, 276032, 276033, 276034, 276035, 276036, 276037, 276038, 276039) c6( ) c7(ci); #c1(ZNF148) c2(NP_068799) c3(zinc finger protein 148) c4(210555, 210556, 210557, 210558, 210559, 210560, 210561) c5(210562, 210563, 210564, 210565, 210566, 210567, 210568, 210569, 210570, 210571, 210572, 210573, 210574, 210575, 210576, 210577, 210578, 210579, 210580, 210581, 210582, 210583, 210584, 210585, 210586, 210587, 210588, 210589, 210590, 210591, 210592, 210593, 210594, 210595, 210596, 210597, 210598, 210599, 210600, 210601, 210602, 210603, 210604, 210605, 210606, 210607, 210608, 210609, 210610, 210611, 210612, 210613, 210614, 210615, 210616) c6( ) c7(dD, B, ea, Y, awP, aq, eq, bC, bz, X, qa, hX); #c1(ZNF154) c2(NP_001078853) c3(zinc finger protein 154) c4(37936, 37937) c5(37938, 37939, 37940, 37941, 37942, 37943, 37944, 37945, 37946, 37947, 37948, 37949, 37950, 37951, 37952, 37953, 37954) c6( ) c7(n, dL, eL); #c1(ZNF160) c2(XP_005259436) c3(zinc finger protein 160 isoform X2) c4( ) c5(276095) c6( ) c7(bpq, ad, aA); #c1(ZNF160) c2(XP_011525767) c3(zinc finger protein 160 isoform X1) c4(320151) c5(320152, 320153, 320154, 320155, 320156, 320157, 320158, 320159, 320160, 320161, 320162, 320163, 320164, 320165) c6( ) c7(bpq, ad, aA); #c1(ZNF160) c2(XP_011525768) c3(zinc finger protein 160 isoform X3) c4(320166) c5(320167, 320168, 320169, 320170, 320171, 320172) c6( ) c7(bpq, ad, aA); #c1(ZNF169) c2(NP_003439) c3(zinc finger protein 169 isoform 1) c4(138400) c5(138401, 138402, 138403, 138404, 138405, 138406, 138407) c6( ) c7(alf, fq); #c1(ZNF169) c2(NP_919301) c3(zinc finger protein 169 isoform 2) c4(258493, 258494) c5(258495, 258496, 258497, 258498, 258499, 258500, 258501, 258502, 258503, 258504, 258505, 258506) c6( ) c7(alf, fq); #c1(ZNF175) c2(NP_009078) c3(zinc finger protein 175) c4(170578) c5(170579, 170580, 170581, 170582, 170583, 170584, 170585) c6( ) c7(amN, fR, h, hh, y, CB); #c1(ZNF177) c2(NP_001166122) c3(zinc finger protein 177 isoform a) c4(68240) c5(68241, 68242, 68243, 68244, 68245, 68246, 68247) c6( ) c7(aA); #c1(ZNF177) c2(NP_003442) c3(zinc finger protein 177 isoform b) c4(138408) c5(138409, 138410, 138411, 138412, 138413, 138414, 138415, 138416, 138417, 138418, 138419, 138420) c6( ) c7(aA); #c1(ZNF182) c2(NP_001007089) c3(zinc finger protein 182 isoform 2) c4(22476) c5(22477, 22478, 22479, 22480, 22481, 22482, 22483) c6( ) c7(iJ); #c1(ZNF182) c2(NP_008893) c3(zinc finger protein 182 isoform 1) c4(169519) c5(169520, 169521, 169522, 169523, 169524, 169525, 169526, 169527, 169528, 169529, 169530, 169531, 169532, 169533) c6( ) c7(iJ); #c1(ZNF184) c2(NP_009080) c3(zinc finger protein 184) c4(170594, 170595, 170596) c5(170597, 170598, 170599, 170600, 170601, 170602, 170603, 170604, 170605, 170606, 170607, 170608, 170609, 170610, 170611, 170612, 170613) c6( ) c7(cm); #c1(ZNF184) c2(XP_011513163) c3(zinc finger protein 184 isoform X2) c4( ) c5(305102, 305103, 305104, 305105, 305106, 305107) c6( ) c7(cm); #c1(ZNF185) c2(NP_001171586) c3(zinc finger protein 185 isoform 9) c4(70646) c5(70647, 70648, 70649, 70650, 70651, 70652, 70653, 70654, 70655) c6( ) c7(av, vA, bz, ae, op, ad, uM); #c1(ZNF185) c2(XP_005274788) c3(zinc finger protein 185 isoform X1) c4(285402) c5(285403, 285404, 285405, 285406, 285407, 285408) c6( ) c7(av, vA, bz, ae, op, ad, uM); #c1(ZNF185) c2(XP_005274790) c3 (zinc finger protein 185 isoform X9) c4( ) c5(285409) c6( ) c7(av, vA, bz, ae, op, ad, uM); #c1(ZNF185) c2(XP_005274792) c3(zinc finger protein 185 isoform X2) c4(285410) c5(285411, 285412, 285413, 285414, 285415, 285416) c6( ) c7(av, vA, bz, ae, op, ad, uM); #c1(ZNF185) c2(XP_005274794) c3(zinc finger protein 185 isoform X3) c4(285417, 285418, 285419) c5(285420, 285421, 285422, 285423, 285424, 285425, 285426, 285427, 285428, 285429, 285430, 285431, 285432, 285433, 285434, 285435, 285436, 285437) c6( ) c7(av, vA, bz, ae, op, ad, uM); #c1(ZNF185) c2(XP_005274795) c3(zinc finger protein 185 isoform X4) c4(285438) c5(285439, 285440, 285441, 285442, 285443, 285444, 285445) c6( ) c7(av, vA, bz, ae, op, ad, uM); #c1(ZNF185) c2(XP_005274796) c3(zinc finger protein 185 isoform X12) c4(285446) c5( ) c6( ) c7(av, vA, bz, ae, op, ad, uM); #c1(ZNF185) c2(XP_005274798) c3(zinc finger protein 185 isoform X5) c4( ) c5(285447) c6( ) c7(av, vA, bz, ae, op, ad, uM); #c1(ZNF185) c2(XP_005274799) c3(zinc finger protein 185 isoform X14) c4(285448) c5(285449, 285450, 285451, 285452, 285453, 285454) c6( ) c7(av, vA, bz, ae, op, ad, uM); #c1(ZNF185) c2(XP_005274801) c3(zinc finger protein 185 isoform X18) c4(285455) c5(285456, 285457, 285458, 285459, 285460, 285461) c6( ) c7(av, vA, bz, ae, op, ad, uM); #c1(ZNF185) c2(XP_005274802) c3(zinc finger protein 185 isoform X6) c4(285462) c5(285463, 285464, 285465, 285466, 285467, 285468) c6( ) c7(av, vA, bz, ae, op, ad, uM); #c1(ZNF185) c2(XP_005274803) c3(zinc finger protein 185 isoform X19) c4(285469) c5(285470, 285471, 285472, 285473, 285474, 285475) c6( ) c7(av, vA, bz, ae, op, ad, uM); #c1(ZNF185) c2(XP_011529496) c3(zinc finger protein 185 isoform X15) c4(324957, 324958) c5(324959, 324960, 324961, 324962, 324963, 324964, 324965, 324966, 324967, 324968, 324969, 324970, 324971, 324972, 324973) c6( ) c7(av, vA, bz, ae, op, ad, uM); #c1(ZNF185) c2(XP_011529498) c3(zinc finger protein 185 isoform X17) c4( ) c5(324974, 324975, 324976, 324977, 324978, 324979) c6( ) c7(av, vA, bz, ae, op, ad, uM); #c1(ZNF189) c2(NP_001265160) c3(zinc finger protein 189 isoform 3) c4(97873) c5(97874, 97875, 97876, 97877, 97878, 97879, 97880, 97881, 97882, 97883, 97884, 97885) c6( ) c7(eL, dL, ff, en); #c1(ZNF189) c2(NP_003443) c3(zinc finger protein 189 isoform 1) c4(138421, 138422) c5(138423, 138424, 138425, 138426, 138427, 138428, 138429) c6( ) c7(eL, dL, ff, en); #c1(ZNF197) c2(NP_001020026) c3(zinc finger protein 197 isoform 2) c4(28063) c5(28064, 28065, 28066, 28067, 28068, 28069, 28070, 28071, 28072, 28073, 28074, 28075, 28076, 28077, 28078, 28079) c6( ) c7(ck, hd, eM, nB, Db, Y, iS, fd, bw, v, K, hF, vt, B, ip); #c1(ZNF197) c2(XP_011547029) c3(zinc finger protein 197 isoform X1) c4(345842, 345843, 345844) c5(345845, 345846, 345847, 345848, 345849, 345850, 345851, 345852, 345853, 345854, 345855, 345856, 345857, 345858, 345859, 345860, 345861) c6( ) c7(ck, hd, eM, nB, Db, Y, iS, fd, bw, v, K, hF, vt, B, ip); #c1(ZNF2) c2(NP_001017396) c3(zinc finger protein 2 isoform b) c4( ) c5(26400, 26401, 26402, 26403, 26404) c6( ) c7(NI); #c1(ZNF2) c2(NP_001269327) c3(zinc finger protein 2 isoform c) c4(101139, 101140) c5(101141, 101142, 101143, 101144, 101145, 101146, 101147) c6( ) c7(NI); #c1(ZNF2) c2(NP_001278533) c3(zinc finger protein 2 isoform d) c4( ) c5(109804, 109805, 109806, 109807, 109808) c6( ) c7(NI); #c1(ZNF2) c2(NP_001278534) c3(zinc finger protein 2 isoform e) c4(109809) c5(109810, 109811, 109812, 109813, 109814, 109815) c6( ) c7(NI); #c1(ZNF2) c2(NP_066574) c3(zinc finger protein 2 isoform a) c4(207441, 207442) c5(207443, 207444, 207445, 207446, 207447, 207448, 207449) c6( ) c7(NI); #c1(ZNF20) c2(NP_066966) c3(zinc finger protein 20 isoform 1) c4(207858, 207859) c5(207860, 207861, 207862, 207863, 207864, 207865, 207866, 207867, 207868, 207869, 207870, 207871, 207872) c6( ) c7(Db, hd, nB); #c1(ZNF202) c2(NP_001288709) c3(zinc finger protein 202 isoform 1) c4(113951) c5(113952, 113953, 113954, 113955, 113956, 113957, 113958, 113959, 113960, 113961, 113962, 113963, 113964, 113965, 113966) c6( ) c7(dD, ea, eD, aw, aA, j, eq); #c1(ZNF205) c2(XP_005255615) c3(zinc finger protein 205 isoform X1) c4(273759, 273760, 273761) c5(273762, 273763, 273764, 273765, 273766, 273767, 273768, 273769, 273770, 273771, 273772, 273773, 273774, 273775, 273776, 273777, 273778, 273779, 273780, 273781, 273782, 273783, 273784) c6( ) c7(kl); #c1(ZNF212) c2(NP_036388) c3(zinc finger protein 212) c4(173493) c5(173494, 173495, 173496, 173497, 173498, 173499, 173500, 173501, 173502) c6( ) c7(j); #c1(ZNF214) c2(XP_006718371) c3(zinc finger protein 214 isoform X1) c4(291791) c5(291792, 291793, 291794, 291795, 291796, 291797, 291798, 291799) c6( ) c7(AV, yi, ND); #c1(ZNF214) c2(XP_011518657) c3(zinc finger protein 214 isoform X2) c4( ) c5(312982, 312983, 312984) c6( ) c7(AV, yi, ND); #c1(ZNF215) c2(NP_037382) c3(zinc finger protein 215) c4(175168) c5(175169, 175170, 175171, 175172, 175173, 175174, 175175, 175176, 175177, 175178, 175179) c6( ) c7(AV, yi, ml, bAF); #c1(ZNF215) c2(XP_011518662) c3(zinc finger protein 215 isoform X2) c4(312985) c5( ) c6( ) c7(AV, yi, ml, bAF); #c1(ZNF215) c2(XP_011518665) c3(zinc finger protein 215 isoform X5) c4( ) c5(312986, 312987, 312988, 312989, 312990, 312991) c6( ) c7(AV, yi, ml, bAF); #c1(ZNF217) c2(NP_006517) c3(zinc finger protein 217) c4(165928, 165929) c5(165930, 165931, 165932, 165933, 165934, 165935, 165936, 165937, 165938, 165939, 165940, 165941, 165942, 165943, 165944, 165945, 165946) c6( ) c7(T, aA, bB, ci, lu, eA, aT, dL, ae, d, s, aW, r, aF, eS, t, ck, bE, auZ, cX, gn, m); #c1(ZNF217) c2(XP_005260602) c3(zinc finger protein 217 isoform X1) c4(276740) c5(276741, 276742, 276743, 276744, 276745, 276746) c6( ) c7(T, aA, bB, ci, lu, eA, aT, dL, ae, d, s, aW, r, aF, eS, t, ck, bE, auZ, cX, gn, m); #c1(ZNF22) c2(NP_008894) c3(zinc finger protein 22) c4(169534) c5(169535, 169536, 169537, 169538, 169539, 169540, 169541) c6( ) c7(cVF); #c1(ZNF224) c2(XP_005259278) c3(zinc finger protein 224 isoform X1) c4(276040, 276041) c5(276042, 276043, 276044, 276045, 276046, 276047, 276048, 276049, 276050, 276051, 276052, 276053, 276054, 276055) c6( ) c7(eL, dL, af, er); #c1(ZNF23) c2(NP_001291421) c3(zinc finger protein 23 isoform a) c4(115976, 115977, 115978) c5(115979, 115980, 115981, 115982, 115983, 115984, 115985, 115986, 115987, 115988, 115989, 115990, 115991, 115992, 115993, 115994, 115995) c6( ) c7(T, B, ac, bt); #c1(ZNF23) c2(NP_001291423) c3(zinc finger protein 23 isoform b) c4( ) c5(115996, 115997, 115998, 115999, 116000, 116001, 116002, 116003, 116004, 116005, 116006, 116007, 116008, 116009) c6( ) c7(T, B, ac, bt); #c1(ZNF230) c2(XP_011525595) c3(zinc finger protein 230 isoform X1) c4(319981) c5(319982, 319983, 319984, 319985, 319986, 319987, 319988) c6( ) c7(vH, hn, at, dL); #c1(ZNF236) c2(NP_031371) c3(zinc finger protein 236) c4(171980) c5(171981, 171982, 171983, 171984, 171985, 171986, 171987) c6( ) c7(cm, aj, K, dy, J); #c1(ZNF236) c2(XP_011524470) c3(zinc finger protein 236 isoform X6) c4(319054) c5(319055, 319056, 319057, 319058, 319059, 319060, 319061, 319062, 319063, 319064) c6( ) c7(cm, aj, K, dy, J); #c1(ZNF239) c2(XP_011538534) c3(zinc finger protein 239 isoform X2) c4( ) c5(336290, 336291, 336292, 336293, 336294) c6( ) c7(bL, bA); #c1(ZNF239) c2(XP_011538539) c3(zinc finger protein 239 isoform X3) c4(336295) c5(336296) c6( ) c7(bL, bA); #c1(ZNF239) c2(XP_011538540) c3(zinc finger protein 239 isoform X1) c4( ) c5(336297, 336298, 336299, 336300, 336301, 336302, 336303, 336304, 336305, 336306, 336307, 336308, 336309, 336310, 336311, 336312, 336313) c6( ) c7(bL, bA); #c1(ZNF24) c2(NP_008896) c3(zinc finger protein 24) c4(169542) c5(169543, 169544, 169545, 169546, 169547, 169548, 169549, 169550, 169551, 169552, 169553) c6( ) c7(T, alC, aW, aT, aq, B); #c1(ZNF248) c2(XP_011517899) c3(zinc finger protein 248 isoform X1) c4(311797, 311798, 311799) c5(311800, 311801, 311802, 311803, 311804, 311805, 311806, 311807, 311808, 311809, 311810) c6( ) c7(nJ, fM, CN, GA); #c1(ZNF248) c2(XP_011517902) c3(zinc finger protein 248 isoform X2) c4(311811) c5(311812, 311813, 311814, 311815, 311816, 311817, 311818, 311819, 311820, 311821, 311822, 311823, 311824, 311825, 311826, 311827, 311828, 311829) c6( ) c7(nJ, fM, CN, GA); #c1(ZNF253) c2(NP_066385) c3(zinc finger protein 253) c4(207279) c5(207280, 207281, 207282, 207283, 207284, 207285, 207286, 207287, 207288, 207289, 207290) c6( ) c7(T, dA, WT, ci, PJ, eW, hX, h, BZ, K, avQ, ak, anR, d, CT, lo, as, pb, ck, Qi, iO, vW, c, iT, eB, U, bC, bt, xg, B); #c1(ZNF260) c2(NP_001012774) c3(zinc finger protein 260) c4(25335) c5(25336, 25337, 25338, 25339, 25340, 25341, 25342, 25343, 25344, 25345) c6( ) c7(ajx, mE, mL, aL, al, aR); #c1(ZNF263) c2(NP_005732) c3(zinc finger protein 263) c4(158938, 158939) c5(158940, 158941, 158942, 158943, 158944, 158945, 158946) c6( ) c7(ea, dD, eq); #c1(ZNF263) c2(XP_006720898) c3(zinc finger protein 263 isoform X5) c4( ) c5(293757, 293758, 293759, 293760, 293761, 293762) c6( ) c7(ea, dD, eq); #c1(ZNF264) c2(NP_003408) c3(zinc finger protein 264) c4(138226) c5(138227, 138228, 138229, 138230, 138231, 138232, 138233, 138234, 138235, 138236, 138237) c6( ) c7(fq, j); #c1(ZNF266) c2(NP_006622) c3(zinc finger protein 266) c4( ) c5(166670, 166671, 166672, 166673, 166674, 166675, 166676, 166677, 166678, 166679, 166680, 166681) c6( ) c7(bP, T, ae, ad, bA); #c1(ZNF266) c2(XP_006722686) c3(zinc finger protein 266 isoform X2) c4(294828) c5(294829, 294830, 294831) c6( ) c7(bP, T, ae, ad, bA); #c1(ZNF267) c2(NP_001252517) c3(zinc finger protein 267 isoform 2) c4( ) c5(94058) c6( ) c7(Pk, gk, al, B, lQ); #c1(ZNF267) c2(NP_003405) c3(zinc finger protein 267 isoform 1) c4(138210, 138211, 138212) c5(138213, 138214, 138215, 138216, 138217, 138218, 138219, 138220, 138221, 138222, 138223, 138224, 138225) c6( ) c7(Pk, gk, al, B, lQ); #c1(ZNF268) c2(NP_001159353) c3(zinc finger protein 268 isoform a) c4(63808, 63809, 63810) c5(63811, 63812, 63813, 63814, 63815, 63816, 63817, 63818, 63819, 63820, 63821, 63822, 63823, 63824, 63825, 63826, 63827, 63828, 63829, 63830, 63831, 63832, 63833, 63834, 63835, 63836) c6( ) c7(ck, if, cn, qN); #c1(ZNF268) c2(NP_001159356) c3(zinc finger protein 268 isoform e) c4(63837) c5(63838, 63839, 63840, 63841, 63842) c6( ) c7(ck, if, cn, qN); #c1(ZNF268) c2(NP_001159357) c3(zinc finger protein 268 isoform f) c4(63843) c5(63844, 63845, 63846, 63847, 63848, 63849, 63850, 63851, 63852) c6( ) c7(ck, if, cn, qN); #c1(ZNF268) c2(NP_001159358) c3(zinc finger protein 268 isoform g) c4(63853) c5(63854, 63855, 63856, 63857, 63858) c6( ) c7(ck, if, cn, qN); #c1(ZNF268) c2(NP_001159359) c3(zinc finger protein 268 isoform h) c4(63859) c5(63860, 63861, 63862, 63863, 63864, 63865, 63866, 63867, 63868) c6( ) c7(ck, if, cn, qN); #c1(ZNF268) c2(NP_694422) c3(zinc finger protein 268 isoform b) c4(245064) c5(245065, 245066, 245067, 245068, 245069, 245070) c6( ) c7(ck, if, cn, qN); #c1(ZNF273) c2(NP_066971) c3(zinc finger protein 273) c4(207892, 207893, 207894, 207895) c5(207896, 207897, 207898, 207899, 207900, 207901, 207902, 207903, 207904, 207905, 207906, 207907, 207908, 207909, 207910, 207911, 207912, 207913, 207914) c6( ) c7(aPm); #c1(ZNF273) c2(XP_011514052) c3(zinc finger protein 273 isoform X2) c4( ) c5(306585, 306586, 306587, 306588, 306589, 306590) c6( ) c7(aPm); #c1(ZNF274) c2(NP_057409) c3(neurotrophin receptor-interacting factor homolog isoform a) c4(190596) c5(190597, 190598, 190599, 190600, 190601, 190602) c6( ) c7(HC); #c1(ZNF274) c2(XP_011524628) c3(neurotrophin receptor-interacting factor homolog isoform X1) c4(319157) c5(319158, 319159, 319160, 319161, 319162, 319163, 319164, 319165) c6( ) c7(HC); #c1(ZNF274) c2(XP_011524629) c3(neurotrophin receptor-interacting factor homolog isoform X2) c4(319166) c5(319167, 319168, 319169, 319170, 319171) c6( ) c7(HC); #c1(ZNF276) c2(NP_001106997) c3(zinc finger protein 276 isoform a) c4(42771, 42772) c5(42773, 42774, 42775, 42776, 42777, 42778, 42779, 42780, 42781, 42782, 42783, 42784, 42785, 42786) c6( ) c7(oZ, os); #c1(ZNF276) c2(XP_005256381) c3(zinc finger protein 276 isoform X2) c4(274163) c5( ) c6( ) c7(oZ, os); #c1(ZNF276) c2(XP_005256385) c3(zinc finger protein 276 isoform X3) c4( ) c5(274164, 274165, 274166, 274167, 274168) c6( ) c7(oZ, os); #c1(ZNF277) c2(NP_068834) c3(zinc finger protein 277) c4(210836, 210837) c5(210838, 210839, 210840, 210841, 210842, 210843, 210844, 210845, 210846, 210847, 210848, 210849, 210850) c6( ) c7(bB, bE, oe); #c1(ZNF280B) c2(XP_011545198) c3(zinc finger protein 280B isoform X1) c4(343978) c5(343979, 343980, 343981, 343982, 343983, 343984, 343985, 343986, 343987, 343988) c6( ) c7(ad); #c1(ZNF280D) c2(NP_001002843) c3(zinc finger protein 280D isoform 2) c4(19642) c5(19643, 19644, 19645, 19646, 19647, 19648, 19649, 19650) c6( ) c7(an); #c1(ZNF280D) c2(NP_001275518) c3(zinc finger protein 2800 isoform 3) c4(106792) c5(106793, 106794, 106795, 106796, 106797, 106798, 106799, 106800, 106801, 106802, 106803, 106804) c6( ) c7(an); #c1(ZNF280D) c2(XP_005254540) c3(zinc finger protein 280D isoform X5) c4(273232) c5(273233, 273234, 273235, 273236, 273237, 273238) c6( ) c7(an); #c1(ZNF280D) c2(XP_005254542) c3(zinc finger protein 2800 isoform X12) c4( ) c5(273239, 273240, 273241, 273242, 273243, 273244) c6( ) c7(an); #c1(ZNF280D) c2(XP_011520005) c3(zinc finger protein 280D isoform X1) c4(314881, 314882, 314883) c5(314884, 314885, 314886, 314887, 314888, 314889, 314890, 314891, 314892) c6( ) c7(an); #c1(ZNF280D) c2(XP_011520012) c3(zinc finger protein 280D isoform X10) c4(314893) c5( ) c6( ) c7(an); #c1(ZNF280D) c2(XP_011520013) c3(zinc finger protein 2800 isoform X11) c4( ) c5(314894, 314895, 314896, 314897, 314898) c6( ) c7(an); #c1(ZNF281) c2(NP_001268222) c3(zinc finger protein 281 isoform 1) c4(99678) c5(99679, 99680, 99681, 99682, 99683, 99684, 99685, 99686, 99687, 99688, 99689) c6( ) c7(n, q, r); #c1(ZNF281) c2(NP_001268223) c3(zinc finger protein 281 isoform 2) c4(99690, 99691) c5(99692, 99693, 99694, 99695, 99696, 99697, 99698, 99699, 99700, 99701, 99702, 99703, 99704) c6( ) c7(n, q, r); #c1(ZNF282) c2(NP_001290410) c3(zinc finger protein 282 isoform 2) c4(115366, 115367) c5(115368, 115369, 115370, 115371, 115372, 115373, 115374, 115375, 115376, 115377, 115378, 115379) c6( ) c7(je, iM, kH); #c1(ZNF282) c2(NP_003566) c3 (zinc finger protein 282 isoform 1) c4(139211) c5(139212, 139213, 139214, 139215, 139216, 139217, 139218) c6( ) c7(je, iM, kH); #c1(ZNF282) c2(XP_011514930) c3(zinc finger protein 282 isoform X2) c4( ) c5(307687) c6( ) c7(je, iM, kH); #c1(ZNF286B) c2(NP_001138517) c3(putative zinc finger protein 286B) c4(56926) c5(56927, 56928, 56929, 56930, 56931, 56932) c6( ) c7(oe); #c1(ZNF296) c2(NP_660331) c3(zinc finger protein 296) c4(240957, 240958) c5(240959, 240960, 240961, 240962, 240963, 240964, 240965, 240966, 240967, 240968, 240969, 240970, 240971) c6( ) c7(ck, x, iT, k, bt, eA, Fc); #c1(ZNF300) c2(NP_001166302) c3(zinc finger protein 300 isoform 1) c4(68548, 68549, 68550, 68551) c5(68552, 68553, 68554, 68555, 68556, 68557, 68558, 68559, 68560) c6( ) c7(bL, ck, A, fM, aW, ff, jh, cj, aT, cz, j, jj, en, hf, y); #c1(ZNF300) c2(XP_006714869) c3(zinc finger protein 300 isoform X1) c4( ) c5(288272, 288273, 288274, 288275, 288276, 288277, 288278, 288279, 288280, 288281, 288282, 288283, 288284, 288285, 288286, 288287) c6( ) c7(bL, ck, A, fM, aW, ff, jh, cj, aT, cz, j, jj, en, hf, y); #c1(ZNF311) c2(NP_001010877) c3(zinc finger protein 311) c4(23909, 23910) c5(23911, 23912, 23913, 23914, 23915, 23916, 23917, 23918, 23919, 23920, 23921, 23922, 23923, 23924, 23925, 23926, 23927, 23928, 23929, 23930, 23931, 23932, 23933, 23934, 23935, 23936, 23937, 23938, 23939, 23940, 23941, 23942, 23943, 23944, 23945, 23946, 23947, 23948, 23949, 23950, 23951, 23952, 23953, 23954, 23955, 23956, 23957, 23958, 23959, 23960, 23961) c6( ) c7(y, eZ); #c1(ZNF311) c2(XP_005249076) c3(zinc finger protein 311 isoform X1) c4(268955) c5( ) c6( ) c7(y, eZ); #c1(ZNF311) c2(XP_006726148) c3(zinc finger protein 311 isoform X7) c4( ) c5(297121, 297122, 297123, 297124, 297125) c6( ) c7(y, eZ); #c1(ZNF311) c2(XP_011546718) c3(zinc finger protein 311 isoform X3) c4(345482) c5( ) c6( ) c7(y, eZ); #c1(ZNF311) c2(XP_011546721) c3(zinc finger protein 311 isoform X6) c4( ) c5(345483) c6( ) c7(y, eZ); #c1(ZNF318) c2(NP_055160) c3(zinc finger protein 318) c4(179526) c5(179527, 179528, 179529, 179530, 179531, 179532, 179533) c6( ) c7(dD, ea, e, v, eq, m, t); #c1(ZNF318) c2(XP_011512754) c3(zinc finger protein 318 isoform X2) c4(303781) c5(303782, 303783, 303784, 303785, 303786, 303787, 303788) c6( ) c7(dD, ea, e, v, eq, m, t); #c1(ZNF32) c2(NP_001005368) c3(zinc finger protein 32) c4(21514) c5(21515, 21516, 21517, 21518, 21519, 21520, 21521, 21522, 21523, 21524, 21525) c6( ) c7(ae, T, fq, jh); #c1(ZNF320) c2(NP_997216) c3(zinc finger protein 320) c4(265192) c5(265193, 265194, 265195, 265196, 265197, 265198, 265199, 265200, 265201, 265202) c6( ) c7(af); #c1(ZNF322) c2(NP_001229728) c3(zinc finger protein 322) c4(84979, 84980, 84981) c5(84982, 84983, 84984, 84985, 84986, 84987, 84988, 84989, 84990, 84991, 84992, 84993, 84994, 84995, 84996, 84997, 84998, 84999, 85000, 85001, 85002, 85003, 85004, 85005, 85006, 85007) c6( ) c7(oe); #c1(ZNF330) c2(NP_001278931) c3(zinc finger protein 330 isoform 2) c4(111063) c5(111064, 111065, 111066, 111067, 111068, 111069) c6( ) c7(Gp, cm, oe, W); #c1(ZNF330) c2(XP_011530177) c3(zinc finger protein 330 isoform X1) c4(326057, 326058, 326059, 326060) c5(326061, 326062, 326063, 326064, 326065, 326066, 326067, 326068, 326069, 326070, 326071, 326072, 326073, 326074, 326075, 326076, 326077, 326078, 326079, 326080, 326081, 326082, 326083, 326084) c6( ) c7(Gp, cm, oe, W); #c1(ZNF331) c2(NP_061025) c3(zinc finger protein 331) c4(198926, 198927, 198928, 198929, 198930, 198931, 198932, 198933, 198934, 198935, 198936, 198937) c5(198938, 198939, 198940, 198941, 198942, 198943, 198944, 198945, 198946, 198947, 198948, 198949, 198950, 198951, 198952, 198953, 198954, 198955, 198956, 198957, 198958, 198959, 198960, 198961, 198962, 198963, 198964, 198965, 198966, 198967, 198968, 198969, 198970, 198971, 198972, 198973, 198974, 198975, 198976, 198977, 198978, 198979, 198980, 198981, 198982, 198983, 198984, 198985, 198986, 198987, 198988, 198989, 198990, 198991, 198992, 198993, 198994, 198995, 198996, 198997, 198998, 198999, 199000, 199001, 199002, 199003, 199004, 199005, 199006, 199007, 199008, 199009, 199010, 199011, 199012, 199013, 199014, 199015, 199016, 199017, 199018, 199019, 199020, 199021, 199022, 199023, 199024, 199025, 199026, 199027, 199028, 199029, 199030, 199031, 199032, 199033, 199034) c6( ) c7(ck, T, eJ, nB, bE, aW, gX, aT, cj, aA, bB, cm, aF, lQ, ci, fd, Pk, bt, B, dx); #c1(ZNF334) c2(XP_011527196) c3(zinc finger protein 334 isoform X5) c4( ) c5(321726) c6( ) c7(y, j); #c1(ZNF334) c2(XP_011527197) c3(zinc finger protein 334 isoform X6) c4( ) c5(321727) c6( ) c7(y, j); #c1(ZNF334) c2(XP_011527198) c3(zinc finger protein 334 isoform X1) c4(321728, 321729) c5(321730, 321731, 321732, 321733, 321734, 321735, 321736) c6( ) c7(y, j); #c1(ZNF334) c2(XP_011527199) c3(zinc finger protein 334 isoform X2) c4( ) c5(321737, 321738, 321739, 321740, 321741) c6( ) c7(y, j); #c1(ZNF335) c2(NP_071378) c3(zinc finger protein 335) c4(211262, 211263, 211264) c5(211265, 211266, 211267, 211268, 211269, 211270, 211271, 211272, 211273, 211274, 211275, 211276, 211277) c6( ) c7(cVG); #c1(ZNF335) c2(XP_005260563) c3(zinc finger protein 335 isoform X2) c4( ) c5(276627, 276628, 276629, 276630, 276631, 276632) c6( ) c7(cVG); #c1(ZNF343) c2(NP_001269424) c3(zinc finger protein 343 isoform 1) c4(101607, 101608) c5(101609, 101610, 101611, 101612, 101613, 101614, 101615, 101616, 101617, 101618, 101619, 101620) c6( ) c7(Ea); #c1(ZNF343) c2(NP_001269426) c3(zinc finger protein 343 isoform 2) c4(101621) c5( ) c6( ) c7(Ea); #c1(ZNF343) c2(NP_001269428) c3(zinc finger protein 343 isoform 4) c4(101622) c5(101623, 101624, 101625, 101626, 101627, 101628, 101629, 101630, 101631, 101632, 101633, 101634) c6( ) c7(Ea); #c1(ZNF35) c2(NP_003411) c3(zinc finger protein 35) c4(138253, 138254, 138255, 138256) c5(138257, 138258, 138259, 138260, 138261, 138262, 138263, 138264, 138265, 138266, 138267, 138268, 138269, 138270, 138271, 138272, 138273, 138274, 138275, 138276, 138277, 138278, 138279, 138280, 138281) c6( ) c7(es, T, aE, iA, aW, Y, aT, Nf, dL, eL, eW); #c1(ZNF350) c2(NP_067645) c3(zinc finger protein 350) c4(209202) c5(209203, 209204, 209205, 209206, 209207, 209208, 209209) c6( ) c7(zQ, dB, aW, if, Qk, aT, kH, at, dL, ci, qN, eA, t, X, ip); #c1(ZNF354A) c2(NP_005640) c3 (zinc finger protein 354A) c4(158191, 158192) c5(158193, 158194, 158195, 158196, 158197, 158198, 158199, 158200, 158201, 158202, 158203, 158204, 158205) c6( ) c7(BI, T, vT, wu, eD, lG, wm, bu, t); #c1(ZNF365) c2(NP_055766) c3(protein ZNF365 isoform A) c4(183365) c5(183366, 183367, 183368, 183369, 183370, 183371) c6( ) c7(cVH, xN, T, PE, aW, HA, aT, aA, fe, K, m, eE, t); #c1(ZNF365) c2(NP_955522) c3(protein ZNF365 isoform B) c4(261856, 261857) c5(261858, 261859, 261860, 261861, 261862, 261863, 261864, 261865, 261866, 261867, 261868, 261869) c6( ) c7(cVH, xN, T, PE, aW, HA, aT, aA, fe, K, m, eE, t); #c1(ZNF365) c2(NP_955523) c3(protein ZNF365 isoform C) c4(261870) c5(261871, 261872, 261873, 261874, 261875, 261876) c6( ) c7(cVH, xN, T, PE, aW, HA, aT, aA, fe, K, m, eE, t); #c1(ZNF365) c2(NP_955524) c3(protein ZNF365 isoform D) c4(261877) c5(261878, 261879, 261880, 261881, 261882, 261883) c6( ) c7(cVH, xN, T, PE, aW, HA, aT, aA, fe, K, m, eE, t); #c1(ZNF366) c2(NP_689838) c3(zinc finger protein 366) c4(244114) c5(244115, 244116, 244117, 244118, 244119, 244120, 244121) c6( ) c7(IB, dD, eq, aT); #c1(ZNF367) c2(NP_710162) c3(zinc finger protein 367) c4(246541) c5(246542, 246543, 246544, 246545, 246546, 246547) c6( ) c7(alf, n, py, d, as); #c1(ZNF382) c2(NP_001243767) c3(zinc finger protein 382 isoform 2) c4(91681) c5(91682, 91683, 91684, 91685, 91686, 91687) c6( ) c7(ci); #c1(ZNF382) c2(NP_116214) c3(zinc finger protein 382 isoform 1) c4(225808) c5(225809, 225810, 225811, 225812, 225813, 225814, 225815) c6( ) c7(ci); #c1(ZNF383) c2(XP_011524892) c3(zinc finger protein 383 isoform X1) c4(319298) c5(319299, 319300, 319301, 319302, 319303, 319304) c6( ) c7(bM); #c1(ZNF384) c2(NP_001035009) c3(zinc finger protein 384 isoform c) c4(32423) c5(32424, 32425, 32426, 32427, 32428, 32429) c6( ) c7(ck, x, ci, k, hF, bA); #c1(ZNF384) c2(NP_001129206) c3(zinc finger protein 384 isoform d) c4(51195, 51196) c5(51197, 51198, 51199, 51200, 51201, 51202, 51203, 51204, 51205, 51206) c6( ) c7(ck, x, ci, k, hF, bA); #c1(ZNF384) c2(NP_597733) c3(zinc finger protein 384 isoform a) c4(235131, 235132) c5(235133, 235134, 235135, 235136, 235137, 235138, 235139, 235140, 235141, 235142, 235143, 235144, 235145, 235146, 235147, 235148, 235149, 235150, 235151, 235152, 235153, 235154, 235155) c6( ) c7(ck, x, ci, k, hF, bA); #c1(ZNF384) c2(XP_011519223) c3(zinc finger protein 384 isoform X1) c4(313810) c5( ) c6( ) c7(ck, x, ci, k, hF, bA); #c1(ZNF384) c2(XP_011519226) c3(zinc finger protein 384 isoform X3) c4( ) c5(313811, 313812, 313813, 313814, 313815, 313816) c6( ) c7(ck, x, ci, k, hF, bA); #c1(ZNF384) c2(XP_011519227) c3(zinc finger protein 384 isoform X4) c4( ) c5(313817, 313818, 313819, 313820, 313821) c6( ) c7(ck, x, ci, k, hF, bA); #c1(ZNF385A) c2(NP_001124439) c3(zinc finger protein 385A isoform a) c4(48896) c5(48897, 48898, 48899, 48900, 48901, 48902, 48903, 48904, 48905) c6( ) c7(IR); #c1(ZNF385A) c2(NP_001124440) c3(zinc finger protein 385A isoform b) c4(48906) c5(48907, 48908, 48909, 48910, 48911, 48912) c6( ) c7(IR); #c1(ZNF385A) c2(NP_001276930) c3(zinc finger protein 385A isoform d) c4( ) c5(108649, 108650, 108651, 108652, 108653) c6( ) c7(IR); #c1(ZNF385A) c2(NP_001276931) c3(zinc finger protein 385A isoform c) c4( ) c5(108654, 108655, 108656, 108657, 108658, 108659, 108660, 108661, 108662, 108663, 108664, 108665, 108666, 108667, 108668, 108669, 108670, 108671, 108672, 108673, 108674, 108675, 108676, 108677, 108678, 108679) c6( ) c7(IR); #c1(ZNF385A) c2(XP_011536470) c3(zinc finger protein 385A isoform X2) c4(333820, 333821, 333822, 333823) c5(333824, 333825, 333826) c6( ) c7(IR); #c1(ZNF385A) c2(XP_011536473) c3(zinc finger protein 385A isoform X5) c4(333827) c5(333828) c6( ) c7(IR); #c1(ZNF385B) c2(NP_001106868) c3(zinc finger protein 385B isoform 2) c4(42644, 42645, 42646) c5(42647, 42648, 42649, 42650, 42651, 42652, 42653, 42654, 42655) c6( ) c7(Na, c, an, aj, v, Nc, m, nA, bX, avQ, bN); #c1(ZNF385B) c2(NP_001106869) c3(zinc finger protein 3858 isoform 3) c4( ) c5(42656, 42657, 42658, 42659, 42660, 42661, 42662, 42663, 42664, 42665, 42666, 42667, 42668) c6( ) c7(Na, c, an, aj, v, Nc, m, nA, bX, avQ, bN); #c1(ZNF385B) c2(NP_689733) c3(zinc finger protein 385B isoform 1) c4(243802, 243803) c5(243804, 243805, 243806, 243807, 243808) c6( ) c7(Na, c, an, aj, v, Nc, m, nA, bX, avQ, bN); #c1(ZNF385B) c2(XP_011509016) c3(zinc finger protein 385B isoform X2) c4(299613) c5( ) c6( ) c7(Na, c, an, aj, v, Nc, m, nA, bX, avQ, bN); #c1(ZNF385B) c2(XP_011509017) c3(zinc finger protein 385B isoform X3) c4( ) c5(299614) c6( ) c7(Na, c, an, aj, v, Nc, m, nA, bX, avQ, bN); #c1(ZNF385B) c2(XP_011509019) c3(zinc finger protein 385B isoform X4) c4(299615) c5( ) c6( ) c7(Na, c, an, aj, v, Nc, m, nA, bX, avQ, bN); #c1(ZNF385B) c2(XP_011509025) c3(zinc finger protein 385B isoform X8) c4( ) c5(299616, 299617, 299618, 299619, 299620, 299621, 299622, 299623, 299624, 299625, 299626, 299627, 299628, 299629, 299630, 299631, 299632, 299633) c6( ) c7(Na, c, an, aj, v, Nc, m, nA, bX, avQ, bN); #c1(ZNF385D) c2(NP_078973) c3(zinc finger protein 385D) c4(216360) c5(216361, 216362, 216363, 216364, 216365, 216366) c6( ) c7(e, oe, VT, lr, j, bK, lB, fq, xa); #c1(ZNF391) c2(XP_011512871) c3(zinc finger protein 391 isoform X1) c4(304153, 304154) c5(304155, 304156, 304157, 304158, 304159, 304160, 304161, 304162, 304163, 304164, 304165, 304166) c6( ) c7(y); #c1(ZNF395) c2(NP_061130) c3(zinc finger protein 395) c4(199272, 199273, 199274, 199275, 199276, 199277) c5(199278, 199279, 199280, 199281, 199282, 199283, 199284, 199285, 199286, 199287, 199288, 199289, 199290, 199291, 199292, 199293, 199294, 199295, 199296, 199297, 199298, 199299, 199300, 199301, 199302, 199303, 199304, 199305, 199306, 199307, 199308, 199309, 199310, 199311, 199312, 199313, 199314, 199315, 199316) c6( ) c7(T, hd, A, nB, iR, aW, oV, eH, eZ, aT, h, hz, cL, aq, oX, bde, pz, eF, mK, o); #c1(ZNF398) c2(NP_065832) c3(zinc finger protein 398 isoform b) c4( ) c5(205728, 205729, 205730, 205731, 205732, 205733, 205734, 205735, 205736, 205737, 205738, 205739, 205740, 205741, 205742, 205743, 205744) c6( ) c7(ae, T, eM, yk, ff, aT, h, m, en, t, bz, B); #c1(ZNF398) c2(NP_733787) c3(zinc finger protein 398 isoform a) c4(247109) c5(247110, 247111, 247112, 247113, 247114, 247115, 247116, 247117, 247118, 247119) c6( ) c7(ae, T, eM, yk, ff, aT, h, m, en, t, bz, B); #c1(ZNF398) c2(XP_011514741) c3(zinc finger protein 398 isoform X3) c4(307386, 307387) c5(307388, 307389, 307390, 307391, 307392, 307393, 307394, 307395) c6( ) c7(ae, T, eM, yk, ff, aT, h, m, en, t, bz, B); #c1(ZNF407) c2(NP_001139661) c3(zinc finger protein 407 isoform 2) c4( ) c5(59636, 59637, 59638, 59639, 59640, 59641) c6( ) c7(fq); #c1(ZNF407) c2(NP_001139662) c3(zinc finger protein 407 isoform 3) c4(59642) c5(59643, 59644, 59645, 59646, 59647, 59648, 59649, 59650, 59651, 59652) c6( ) c7(fq); #c1(ZNF407) c2(NP_060227) c3(zinc finger protein 407 isoform 1) c4(194861, 194862) c5(194863, 194864, 194865, 194866, 194867, 194868) c6( ) c7(fq); #c1(ZNF41) c2(NP_700359) c3(zinc finger protein 41) c4(245925) c5(245926, 245927, 245928, 245929, 245930, 245931, 245932, 245933, 245934, 245935, 245936) c6( ) c7(nd, nf, nA, xg, he, iJ); #c1(ZNF41) c2(XP_006724618) c3(zinc finger protein 41 isoform X4) c4(296120, 296121) c5(296122, 296123, 296124, 296125, 296126, 296127, 296128, 296129, 296130, 296131, 296132, 296133) c6( ) c7(nd, nf, nA, xg, he, iJ); #c1(ZNF410) c2(NP_001229853) c3(zinc finger protein 410 isoform a) c4(85421) c5(85422, 85423, 85424, 85425, 85426, 85427) c6( ) c7(T, A, eR, ig, bE, aW, hl, r, aT, uz, bB, at, qj, fe, ER, ei, bw, HI, q, pC); #c1(ZNF410) c2(NP_001229855) c3(zinc finger protein 410 isoform c) c4(85434, 85435, 85436, 85437) c5(85438, 85439, 85440, 85441, 85442, 85443, 85444, 85445, 85446, 85447, 85448, 85449, 85450, 85451, 85452, 85453, 85454, 85455, 85456, 85457, 85458, 85459, 85460, 85461, 85462, 85463, 85464, 85465, 85466) c6( ) c7(T, A, eR, ig, bE, aW, hl, r, aT, uz, bB, at, qj, fe, ER, ei, bw, HI, q, pC); #c1(ZNF410) c2(NP_001229856) c3(zinc finger protein 410 isoform d) c4(85467) c5(85468, 85469, 85470, 85471, 85472, 85473) c6( ) c7(T, A, eR, ig, bE, aW, hl, r, aT, uz, bB, at, qj, fe, ER, ei, bw, HI, q, pC); #c1(ZNF410) c2(NP_001229857) c3(zinc finger protein 410 isoform e) c4( ) c5(85474, 85475) c6( ) c7(T, A, eR, ig, bE, aW, hl, r, aT, uz, bB, at, qj, fe, ER, ei, bw, HI, q, pC); #c1(ZNF410) c2(NP_067011) c3(zinc finger protein 410 isoform b) c4(208328, 208329, 208330, 208331) c5(208332, 208333, 208334, 208335, 208336, 208337, 208338, 208339, 208340, 208341, 208342, 208343, 208344, 208345, 208346, 208347, 208348, 208349, 208350) c6( ) c7(T, A, eR, ig, bE, aW, hl, r, aT, uz, bB, at, qj, fe, ER, ei, bw, HI, q, pC); #c1(ZNF415) c2(NP_001129510) c3(zinc finger protein 415 isoform 1) c4(51786) c5(51787, 51788, 51789, 51790, 51791, 51792) c6( ) c7(T); #c1(ZNF415) c2(NP_060825) c3(zinc finger protein 415 isoform 2) c4( ) c5(198007, 198008, 198009, 198010, 198011) c6( ) c7(T); #c1(ZNF415) c2(XP_006723331) c3(zinc finger protein 415 isoform X5) c4(295129) c5(295130) c6( ) c7(T); #c1(ZNF415) c2(XP_006723333) c3(zinc finger protein 415 isoform X2) c4( ) c5(295131) c6( ) c7(T); #c1(ZNF415) c2(XP_006723337) c3(zinc finger protein 415 isoform X8) c4(295132, 295133) c5(295134, 295135, 295136, 295137, 295138, 295139, 295140, 295141, 295142, 295143, 295144, 295145, 295146, 295147, 295148, 295149) c6( ) c7(T); #c1(ZNF415) c2(XP_011525400) c3(zinc finger protein 415 isoform X3) c4(319845) c5( ) c6( ) c7(T); #c1(ZNF415) c2(XP_011525408) c3(zinc finger protein 415 isoform X6) c4( ) c5(319846, 319847, 319848, 319849, 319850) c6( ) c7(T); #c1(ZNF419) c2(NP_001091961) c3(zinc finger protein 419 isoform 1) c4(39019, 39020) c5(39021, 39022, 39023, 39024, 39025, 39026, 39027, 39028, 39029, 39030, 39031, 39032) c6( ) c7(fr); #c1(ZNF419) c2(NP_001091962) c3(zinc finger protein 419 isoform 3) c4(39033, 39034, 39035) c5(39036, 39037, 39038, 39039, 39040, 39041, 39042, 39043, 39044, 39045, 39046, 39047, 39048, 39049, 39050) c6( ) c7(fr); #c1(ZNF419) c2(NP_001091963) c3(zinc finger protein 419 isoform 4) c4(39051, 39052) c5(39053, 39054, 39055, 39056, 39057, 39058, 39059, 39060, 39061, 39062, 39063, 39064, 39065, 39066, 39067) c6( ) c7(fr); #c1(ZNF419) c2(NP_001091964) c3(zinc finger protein 419 isoform 5) c4(39068) c5(39069, 39070, 39071, 39072, 39073, 39074) c6( ) c7(fr); #c1(ZNF419) c2(NP_001091965) c3(zinc finger protein 419 isoform 6) c4(39075) c5(39076, 39077, 39078, 39079, 39080) c6( ) c7(fr); #c1(ZNF419) c2(NP_001091966) c3(zinc finger protein 419 isoform 7) c4(39081) c5(39082, 39083, 39084, 39085, 39086, 39087, 39088) c6( ) c7(fr); #c1(ZNF419) c2(NP_001278672) c3(zinc finger protein 419 isoform 8) c4(109963) c5(109964, 109965, 109966, 109967, 109968, 109969) c6( ) c7(fr); #c1(ZNF419) c2(NP_078967) c3(zinc finger protein 419 isoform 2) c4(216319) c5(216320, 216321, 216322, 216323, 216324, 216325) c6( ) c7(fr); #c1(ZNF423) c2(NP_001258549) c3(zinc finger protein 423 isoform 2) c4( ) c5(96126, 96127, 96128, 96129, 96130, 96131) c6( ) c7(bM, iU, aW, cVl, eZ, aT, cm, Xr, hF, xa, y); #c1(ZNF423) c2(NP_055884) c3(zinc finger protein 423 isoform 1) c4(184158) c5(184159, 184160, 184161, 184162, 184163, 184164, 184165, 184166) c6( ) c7(bM, iU, aW, cVl, eZ, aT, cm, Xr, hF, xa, y); #c1 (ZNF423) c2(XP_005255914) c3(zinc finger protein 423 isoform X4) c4(273894) c5( ) c6( ) c7(bM, iU, aW, cVl, eZ, aT, cm, Xr, hF, xa, y); #c1(ZNF423) c2(XP_011521264) c3(zinc finger protein 423 isoform X2) c4( ) c5(316172, 316173) c6( ) c7(bM, iU, aW, cVl, eZ, aT, cm, Xr, hF, xa, y); #c1 (ZNF430) c2(NP_001166142) c3(zinc finger protein 430 isoform 2) c4(68305, 68306) c5(68307, 68308, 68309, 68310, 68311, 68312, 68313, 68314, 68315, 68316, 68317, 68318) c6( ) c7(kl); #c1(ZNF430) c2(NP_079465) c3(zinc finger protein 430 isoform 1) c4(218201) c5(218202, 218203, 218204, 218205, 218206, 218207) c6( ) c7(kl); #c1(ZNF432) c2(XP_011525850) c3(zinc finger protein 432 isoform X1) c4(320272) c5(320273, 320274, 320275, 320276, 320277, 320278, 320279) c6( ) c7(fe, aT); #c1(ZNF433) c2(NP_001073880) c3(zinc finger protein 433) c4( ) c5(36214, 36215, 36216, 36217, 36218, 36219) c6( ) c7(V); #c1(ZNF433) c2(XP_011526024) c3(zinc finger protein 433 isoform X1) c4(320369) c5( ) c6( ) c7(V); #c1(ZNF433) c2(XP_011526025) c3(zinc finger protein 433 isoform X2) c4(320370) c5( ) c6( ) c7(V); #c1(ZNF433) c2(XP_011526027) c3(zinc finger protein 433 isoform X4) c4( ) c5(320371) c6( ) c7(V); #c1(ZNF438) c2(NP_001137240) c3(zinc finger protein 438 isoform a) c4(55104, 55105, 55106) c5(55107, 55108, 55109, 55110, 55111, 55112, 55113, 55114, 55115, 55116, 55117, 55118, 55119, 55120, 55121, 55122, 55123, 55124, 55125, 55126, 55127) c6( ) c7(Y); #c1(ZNF438) c2(NP_00137241) c3(zinc finger protein 438 isoform c) c4( ) c5(55128, 55129, 55130, 55131, 55132, 55133) c6( ) c7(Y); #c1(ZNF438) c2(NP_001137243) c3(zinc finger protein 438 isoform b) c4(55134, 55135) c5(55136, 55137, 55138, 55139, 55140, 55141, 55142, 55143, 55144, 55145, 55146, 55147, 55148, 55149, 55150, 55151, 55152, 55153, 55154, 55155, 55156, 55157, 55158, 55159) c6( ) c7(Y); #c1(ZNF44) c2(NP_001157748) c3(zinc finger protein 44 isoform 1) c4(62525, 62526) c5(62527, 62528, 62529, 62530, 62531, 62532, 62533, 62534, 62535, 62536, 62537, 62538) c6( ) c7(ck, cOR, fb); #c1(ZNF44) c2(NP_057348) c3(zinc finger protein 44 isoform 2) c4(190253, 190254) c5(190255, 190256, 190257, 190258, 190259, 190260, 190261, 190262, 190263, 190264, 190265, 190266, 190267, 190268) c6( ) c7(ck, cOR, fb); #c1(ZNF44) c2(XP_011526364) c3(zinc finger protein 44 isoform X1) c4( ) c5(320658, 320659, 320660) c6( ) c7(ck, cOR, fb); #c1(ZNF44) c2(XP_011526365) c3(zinc finger protein 44 isoform X2) c4( ) c5(320661) c6( ) c7(ck, cOR, fb); #c1(ZNF443) c2(NP_005806) c3(zinc finger protein 443) c4(159595, 159596) c5(159597, 159598, 159599, 159600, 159601, 159602, 159603, 159604, 159605, 159606, 159607, 159608, 159609) c6( ) c7(B); #c1(ZNF444) c2(XP_011525370) c3(zinc finger protein 444 isoform X1) c4(319819) c5(319820, 319821, 319822, 319823, 319824, 319825) c6( ) c7(YP); #c1(ZNF45) c2(NP_003416) c3(zinc finger protein 45) c4(138282, 138283) c5(138284, 138285, 138286, 138287, 138288, 138289, 138290, 138291, 138292, 138293, 138294, 138295) c6( ) c7(V); #c1(ZNF451) c2(NP_001026794) c3(zinc finger protein 451 isoform 1) c4(29633) c5(29634, 29635, 29636, 29637, 29638, 29639) c6( ) c7(ck, ae, kS, ad, j); #c1(ZNF451) c2(NP_001244202) c3(zinc finger protein 451 isoform 3) c4(92025) c5(92026, 92027, 92028, 92029, 92030, 92031, 92032) c6( ) c7(ck, ae, kS, ad, j); #c1(ZNF451) c2(NP_056370) c3(zinc finger protein 451 isoform 2) c4(187327) c5(187328, 187329, 187330, 187331, 187332, 187333) c6( ) c7(ck, ae, kS, ad, j); #c1(ZNF462) c2(NP_067047) c3(zinc finger protein 462) c4(208713) c5(208714, 208715, 208716, 208717, 208718, 208719) c6( ) c7(ad); #c1(ZNF462) c2(XP_006717278) c3(zinc finger protein 462 isoform X3) c4(290858) c5(290859, 290860, 290861, 290862, 290863, 290864) c6( ) c7(ad); #c1(ZNF462) c2(XP_006717281) c3(zinc finger protein 462 isoform X4) c4(290865, 290866) c5(290867, 290868, 290869, 290870, 290871, 290872, 290873, 290874, 290875, 290876, 290877, 290878, 290879) c6( ) c7(ad); #c1(ZNF469) c2(NP_001120936) c3(zinc finger protein 469) c4(44974) c5(44975, 44976, 44977, 44978, 44979, 44980, 44981) c6( ) c7(aTf, nX, ev, cTV, la, ab, K, aqf, aOm, aOl); #c1(ZNF483) c2(NP_001007170) c3(zinc finger protein 483 isoform b) c4(22525) c5(22526, 22527, 22528, 22529, 22530, 22531, 22532, 22533, 22534, 22535, 22536, 22537, 22538, 22539, 22540, 22541, 22542, 22543, 22544) c6( ) c7(v); #c1(ZNF483) c2(NP_597721) c3(zinc finger protein 483 isoform a) c4(235103, 235104, 235105) c5(235106, 235107, 235108, 235109, 235110, 235111) c6( ) c7(v); #c1(ZNF490) c2(NP_065765) c3(zinc finger protein 490) c4(205267) c5(205268, 205269, 205270, 205271, 205272, 205273, 205274) c6( ) c7(bK); #c1(ZNF492) c2(NP_065906) c3(zinc finger protein 492) c4(206157) c5(206158, 206159, 206160, 206161, 206162, 206163, 206164, 206165, 206166, 206167, 206168) c6( ) c7(ab, aw); #c1(ZNF496) c2(NP_116141) c3(zinc finger protein 496) c4(225472) c5(225473, 225474, 225475, 225476, 225477, 225478, 225479) c6( ) c7(c); #c1(ZNF496) c2(XP_005273385) c3(zinc finger protein 496 isoform X4) c4(284549) c5(284550, 284551, 284552, 284553, 284554, 284555) c6( ) c7(c); #c1(ZNF501) c2(NP_659481) c3(zinc finger protein 501) c4(240137, 240138) c5(240139, 240140, 240141, 240142, 240143, 240144, 240145, 240146, 240147, 240148, 240149, 240150) c6( ) c7(ck, cOR, fb); #c1(ZNF507) c2(NP_001129628) c3(zinc finger protein 507) c4(52077, 52078) c5(52079, 52080, 52081, 52082, 52083, 52084, 52085, 52086, 52087, 52088, 52089, 52090, 52091, 52092, 52093, 52094) c6( ) c7(he, bN); #c1(ZNF512) c2(NP_001258215) c3(zinc finger protein 512 isoform b) c4(95992) c5(95993, 95994, 95995, 95996, 95997, 95998, 95999) c6( ) c7(om); #c1(ZNF512) c2(NP_001258217) c3(zinc finger protein 512 isoform c) c4( ) c5(96000, 96001, 96002, 96003, 96004) c6( ) c7(om); #c1(ZNF512) c2(NP_001258218) c3(zinc finger protein 512 isoform d) c4( ) c5(96005, 96006, 96007, 96008, 96009) c6( ) c7(om); #c1(ZNF512) c2(NP_115810) c3(zinc finger protein 512 isoform a) c4(224017, 224018, 224019, 224020) c5(224021, 224022, 224023, 224024, 224025, 224026, 224027, 224028, 224029, 224030, 224031, 224032, 224033) c6( ) c7(om); #c1(ZNF512B) c2(NP_065764) c3(zinc finger protein 512B) c4( ) c5(205256, 205257, 205258, 205259, 205260, 205261, 205262, 205263, 205264, 205265, 205266) c6( ) c7(c); #c1(ZNF512B) c2(XP_005260283) c3(zinc finger protein 512B isoform X2) c4(276508) c5(276509, 276510, 276511) c6( ) c7(c); #c1(ZNF513) c2(NP_001188388) c3(zinc finger protein 513 isoform 2) c4( ) c5(79106, 79107, 79108, 79109, 79110) c6( ) c7(dB, uz, cVJ, nk); #c1(ZNF513) c2(NP_653232) c3(zinc finger protein 513 isoform 1) c4(239248, 239249) c5(239250, 239251, 239252, 239253, 239254, 239255, 239256, 239257) c6( ) c7(dB, uz, cVJ, nk); #c1(ZNF516) c2(NP_055458) c3(zinc finger protein 516) c4(181305) c5(181306, 181307, 181308, 181309, 181310, 181311, 181312, 181313, 181314, 181315, 181316) c6( ) c7(q, r); #c1(ZNF516) c2(XP_011524579) c3(zinc finger protein 516 isoform X3) c4(319147) c5(319148) c6( ) c7(q, r); #c1(ZNF519) c2(XP_011523906) c3(zinc finger protein 519 isoform X1) c4(318433) c5(318434, 318435, 318436, 318437, 318438, 318439, 318440, 318441, 318442, 318443) c6( ) c7(c); #c1(ZNF521) c2(NP_056276) c3(zinc finger protein 521) c4(186869, 186870, 186871) c5(186872, 186873, 186874, 186875, 186876, 186877, 186878, 186879, 186880, 186881, 186882, 186883, 186884, 186885, 186886, 186887, 186888, 186889, 186890) c6( ) c7(dB, x, jf, ck, k); #c1(ZNF536) c2(NP_055532) c3(zinc finger protein 536) c4(181806) c5(181807, 181808, 181809, 181810, 181811) c6( ) c7(e, fq, fr); #c1(ZNF536) c2(XP_011525861) c3(zinc finger protein 536 isoform X2) c4( ) c5(320287) c6( ) c7(e, fq, fr); #c1(ZNF555) c2(NP_001166246) c3(zinc finger protein 555 isoform 2) c4(68529) c5(68530, 68531, 68532, 68533, 68534, 68535) c6( ) c7(ac); #c1(ZNF555) c2(NP_690004) c3(zinc finger protein 555 isoform 1) c4(244704) c5(244705, 244706, 244707, 244708, 244709, 244710) c6( ) c7(ac); #c1(ZNF559) c2(NP_001189335) c3(zinc finger protein 559 isoform a) c4(79283, 79284) c5(79285, 79286, 79287) c6( ) c7(aF, aA); #c1(ZNF559) c2(NP_001189336) c3(zinc finger protein 559 isoform c) c4( ) c5(79288, 79289, 79290, 79291, 79292, 79293) c6( ) c7(aF, aA); #c1(ZNF559) c2(NP_115886) c3(zinc finger protein 559 isoform b) c4( ) c5(224522, 224523, 224524, 224525, 224526, 224527, 224528, 224529, 224530, 224531, 224532) c6( ) c7(aF, aA); #c1(ZNF568) c2(NP_001191766) c3(zinc finger protein 568 isoform 3) c4( ) c5(81739, 81740, 81741, 81742, 81743) c6( ) c7(j); #c1(ZNF568) c2(NP_001191767) c3(zinc finger protein 568 isoform 5) c4(81744) c5(81745, 81746, 81747, 81748, 81749, 81750, 81751, 81752, 81753, 81754, 81755, 81756) c6( ) c7(j); #c1(ZNF568) c2(NP_001191768) c3(zinc finger protein 568 isoform 6) c4(81757) c5( ) c6( ) c7(j); #c1(ZNF568) c2(NP_940941) c3(zinc finger protein 568 isoform 1) c4(260158, 260159) c5(260160, 260161, 260162, 260163, 260164, 260165, 260166) c6( ) c7(j); #c1(ZNF569) c2(NP_689697) c3(zinc finger protein 569) c4( ) c5(243604, 243605, 243606, 243607, 243608, 243609, 243610, 243611, 243612, 243613, 243614, 243615) c6( ) c7(T, dA, WT, ci, PJ, eW, hX, h, BZ, K, cOR, fb, avQ, ak, anR, d, CT, lo, as, pb, ck, Qi, iO, vW, c, iT, eB, U, bC, bt, xg, B); #c1(ZNF569) c2(XP_005258621) c3(zinc finger protein 569 isoform X3) c4( ) c5(275702, 275703, 275704, 275705, 275706) c6( ) c7(T, dA, WT, ci, PJ, eW, hX, h, BZ, K, cOR, fb, avQ, ak, anR, d, CT, lo, as, pb, ck, Qi, iO, vW, c, iT, eB, U, bC, bt, xg, B); #c1(ZNF569) c2(XP_006723111) c3(zinc finger protein 569 isoform X2) c4(295011, 295012, 295013) c5(295014) c6( ) c7(T, dA, WT, ci, PJ, eW, hX, h, BZ, K, cOR, fb, avQ, ak, anR, d, CT, lo, as, pb, ck, Qi, iO, vW, c, iT, eB, U, bC, bt, xg, B); #c1(ZNF577) c2(NP_001129062) c3(zinc finger protein 577 isoform b) c4( ) c5(50705, 50706, 50707, 50708, 50709, 50710, 50711, 50712, 50713, 50714, 50715, 50716, 50717, 50718, 50719, 50720, 50721, 50722) c6( ) c7(g, ff, pm, en); #c1(ZNF577) c2(XP_011525714) c3(zinc finger protein 577 isoform X1) c4(320123, 320124, 320125, 320126, 320127) c5(320128, 320129, 320130, 320131, 320132, 320133, 320134, 320135, 320136, 320137, 320138, 320139) c6( ) c7(g, ff, pm, en); #c1(ZNF580) c2(NP_057286) c3(zinc finger protein 580) c4( ) c5(189853, 189854, 189855, 189856, 189857, 189858, 189859, 189860, 189861, 189862, 189863, 189864, 189865, 189866) c6( ) c7(j); #c1(ZNF580) c2(XP_005259024) c3(zinc finger protein 580 isoform X4) c4(275924) c5( ) c6( ) c7(j); #c1(ZNF581) c2(XP_006723303) c3(zinc finger protein 581 isoform X1) c4(295108) c5(295109, 295110, 295/11, 295112, 295113, 295114, 295115) c6( ) c7(j); #c1(ZNF582) c2(NP_653291) c3(zinc finger protein 582) c4( ) c5(239518, 239519, 239520, 239521, 239522) c6( ) c7(if, QO, qN); #c1(ZNF582) c2(XP_006723107) c3(zinc finger protein 582 isoform X1) c4(295009) c5(295010) c6( ) c7(if, QO, qN); #c1(ZNF583) c2(XP_011524819) c3(zinc finger protein 583 isoform X1) c4(319228, 319229, 319230) c5(319231, 319232, 319233, 319234, 319235, 319236, 319237, 319238, 319239, 319240, 319241, 319242, 319243, 319244, 319245, 319246, 319247, 319248, 319249, 319250, 319251, 319252) c6( ) c7(r); #c1(ZNF585B) c2(NP_689492) c3(zinc finger protein 585B) c4(242895, 242896, 242897) c5(242898, 242899, 242900, 242901, 242902, 242903, 242904, 242905, 242906, 242907, 242908, 242909, 242910, 242911, 242912, 242913, 242914, 242915, 242916, 242917) c6( ) c7(bM); #c1(ZNF592) c2(NP_055445) c3(zinc finger protein 592) c4(181179) c5(181180, 181181, 181182, 181183, 181184, 181185, 181186, 181187, 181188, 181189, 181190, 181191) c6( ) c7(gZ, bFu, nA, cVK); #c1(ZNF596) c2(XP_011533044) c3(zinc finger protein 596 isoform X1) c4(329251, 329252) c5(329253, 329254, 329255, 329256, 329257, 329258, 329259, 329260, 329261, 329262) c6( ) c7(bX); #c1(ZNF596) c2(XP_011533046) c3(zinc finger protein 596 isoform X2) c4( ) c5(329263, 329264, 329265, 329266, 329267, 329268) c6( ) c7(bX); #c1(ZNF606) c2(NP_079303) c3(zinc finger protein 606) c4(217709, 217710, 217711) c5(217712, 217713, 217714, 217715, 217716, 217717, 217718, 217719, 217720, 217721, 217722, 217723, 217724, 217725, 217726, 217727, 217728, 217729) c6( ) c7(IB); #c1(ZNF607) c2(NP_001166148) c3(zinc finger protein 607 isoform 2) c4(68339) c5(68340, 68341, 68342, 68343, 68344, 68345) c6( ) c7(ac); #c1(ZNF607) c2(NP_116078) c3(zinc finger protein 607 isoform 1) c4(225332) c5(225333, 225334, 225335, 225336, 225337, 225338) c6( ) c7(ac); #c1(ZNF608) c2(NP_065798) c3(zinc finger protein 608) c4(205515, 205516) c5(205517, 205518, 205519, 205520, 205521, 205522, 205523, 205524, 205525, 205526, 205527, 205528, 205529, 205530, 205531, 205532, 205533, 205534, 205535, 205536, 205537, 205538, 205539) c6( ) c7(v, cd); #c1(ZNF608) c2(XP_005272096) c3(zinc finger protein 608 isoform X2) c4(283674) c5( ) c6( ) c7(v, cd); #c1(ZNF608) c2(XP_011541822) c3(zinc finger protein 608 isoform X3) c4(340392) c5( ) c6( ) c7(v, cd); #c1(ZNF608) c2(XP_011541823) c3(zinc finger protein 608 isoform X4) c4( ) c5(340393) c6( ) c7(v, cd); #c1(ZNF615) c2(XP_011525128) c3(zinc finger protein 615 isoform X1) c4(319542, 319543, 319544) c5(319545, 319546, 319547, 319548, 319549, 319550, 319551, 319552, 319553, 319554, 319555, 319556, 319557, 319558, 319559, 319560, 319561, 319562) c6( ) c7(an); #c1(ZNF615) c2(XP_011525129) c3(zinc finger protein 615 isoform X4) c4(319563) c5(319564, 319565, 319566, 319567, 319568, 319569) c6( ) c7(an); #c1(ZNF615) c2(XP_011525130) c3(zinc finger protein 615 isoform X2) c4(319570) c5(319571, 319572, 319573, 319574, 319575, 319576) c6( ) c7(an); #c1(ZNF618) c2(NP_588615) c3(zinc finger protein 618) c4(234825) c5(234826, 234827, 234828, 234829, 234830, 234831) c6( ) c7(Na, Nc, oe, dy, ac); #c1(ZNF618) c2(XP_011516505) c3 (zinc finger protein 618 isoform X9) c4(309901, 309902) c5(309903, 309904, 309905, 309906, 309907, 309908, 309909, 309910, 309911, 309912, 309913, 309914) c6( ) c7(Na, Nc, oe, dy, ac); #c1(ZNF627) c2(NP_660338) c3(zinc finger protein 627 isoform 1) c4(241000) c5(241001, 241002, 241003, 241004, 241005, 241006) c6( ) c7(cH, aw); #c1(ZNF629) c2(NP_001073886) c3(zinc finger protein 629) c4(36309) c5(36310, 36311, 36312, 36313, 36314, 36315) c6( ) c7(T, dA, WT, ci, PJ, eW, hX, h, BZ, K, cOR, fb, avQ, ak, anR, d, CT, lo, as, pb, ck, Qi, iO, vW, c, iT, eB, U, bC, bt, xg, B); #c1(ZNF639) c2(NP_001290355) c3(zinc finger protein 639) c4(115241, 115242, 115243, 115244, 115245) c5(115246, 115247, 115248, 115249, 115250, 115251, 115252, 115253, 115254, 115255, 115256, 115257, 115258, 115259, 115260, 115261, 115262, 115263, 115264, 115265, 115266, 115267, 115268, 115269, 115270, 115271, 115272, 115273, 115274, 115275, 115276, 115277, 115278, 115279, 115280, 115281, 115282, 115283, 115284) c6( ) c7(h, oe, ff, en); #c1(ZNF644) c2(XP_011540563) c3(zinc finger protein 644 isoform X1) c4(339050) c5( ) c6( ) c7(cVL, asg); #c1(ZNF645) c2(NP_689790) c3(E3 ubiquitin-protein ligase ZNF645) c4(243976) c5(243977, 243978, 243979, 243980, 243981, 243982, 243983) c6( ) c7(cnT, T, os, dt, fM, hl, yS, al, jh, bB, MF, aj, v, BF, ci, C, nA, oZ, aY, yU, fr, q, J, ad, hX, eA, eW, oT, bgk, aE, lg, py, rw, aT, h, yT, yZ, bl, K, jj, bU, an, pq, ae, cnS, pi, bF, ak, JI, aW, Y, Ls, cr, bTw, nf, aiT, dq, lo, r, aq, Tp, iR, bw, bz, bN, hq, ck, dS, es, bAn, alC, jV, eM, iA, bE, iT, agl, U, aS, art, y, bt, kS, B, VB); #c1(ZNF646) c2(NP_055514) c3(zinc finger protein 646) c4(181637, 181638, 181639) c5(181640, 181641, 181642, 181643, 181644, 181645, 181646, 181647, 181648, 181649, 181650, 181651) c6( ) c7(an); #c1(ZNF646) c2(XP_005255769) c3(zinc finger protein 646 isoform X2) c4(273850) c5(273851, 273852, 273853, 273854, 273855, 273856, 273857, 273858, 273859, 273860, 273861, 273862, 273863, 273864) c6( ) c7(an); #c1(ZNF652) c2(NP_001138837) c3(zinc finger protein 652) c4(57912) c5(57913, 57914, 57915, 57916, 57917, 57918, 57919, 57920, 57921, 57922) c6( ) c7(T, aW, oe, cz, aT, ad); #c1(ZNF652) c2(XP_005257223) c3(zinc finger protein 652 isoform X1) c4(274806) c5(274807, 274808, 274809, 274810, 274811, 274812) c6( ) c7(T, aW, oe, cz, aT, ad); #c1(ZNF654) c2(NP_060763) c3(zinc finger protein 654) c4(197671) c5(197672, 197673, 197674, 197675, 197676, 197677) c6( ) c7(T, dA, v, ci, eA, q, hd, aT, d, aW, Y, oV, ff, r, aq, en, bu, t, bz, iP, eM, nB, eD, B); #c1(ZNF664) c2(NP_689650) c3(zinc finger protein 664) c4(243440, 243441) c5(243442, 243443, 243444, 243445, 243446, 243447, 243448, 243449, 243450, 243451, 243452, 243453, 243454, 243455, 243456, 243457, 243458, 243459, 243460, 243461, 243462, 243463, 243464, 243465) c6( ) c7(aw); #c1(ZNF667) c2(NP_071386) c3(zinc finger protein 667) c4(211313, 211314, 211315, 211316) c5(211317, 211318, 211319, 211320, 211321, 211322, 211323, 211324, 211325, 211326, 211327, 211328, 211329, 211330, 211331, 211332) c6( ) c7(de); #c1(ZNF667) c2(XP_011525513) c3(zinc finger protein 667 isoform X2) c4( ) c5(319929, 319930, 319931, 319932, 319933, 319934, 319935, 319936, 319937, 319938, 319939, 319940) c6( ) c7(oe); #c1(ZNF668) c2(NP_078982) c3(zinc finger protein 668 isoform a) c4(216396, 216397) c5(216398, 216399, 216400, 216401, 216402, 216403, 216404, 216405, 216406, 216407, 216408, 216409, 216410, 216411, 216412, 216413, 216414, 216415, 216416, 216417, 216418, 216419, 216420, 216421, 216422, 216423) c6( ) c7(aW, XV, aT); #c1(ZNF668) c2(XP_011544251) c3(zinc finger protein 668 isoform X1) c4(343400, 343401, 343402, 343403) c5(343404, 343405, 343406, 343407, 343408, 343409, 343410, 343411, 343412, 343413, 343414, 343415, 343416, 343417, 343418, 343419, 343420, 343421, 343422, 343423, 343424, 343425, 343426, 343427, 343428, 343429, 343430, 343431) c6( ) c7(aW, XV, aT); #c1(ZNF674) c2(NP_001139763) c3(zinc finger protein 674 isoform 2) c4(59908) c5(59909, 59910, 59911, 59912, 59913, 59914) c6( ) c7(nf, nx, nA); #c1(ZNF674) c2(NP_001177346) c3(zinc finger protein 674 isoform 3) c4( ) c5(72694) c6( ) c7(nf, nx, nA); #c1(ZNF674) c2(XP_011542244) c3(zinc finger protein 674 isoform X1) c4(340755) c5(340756, 340757, 340758, 340759, 340760, 340761, 340762, 340763) c6( ) c7(nf, nx, nA); #c1(ZNF674) c2(XP_011542245) c3(zinc finger protein 674 isoform X2) c4( ) c5(340764, 340765) c6( ) c7(nf, nx, nA); #c1(ZNF676) c2(XP_011526034) c3(zinc finger protein 676 isoform X1) c4( ) c5(320372) c6( ) c7(hz); #c1(ZNF676) c2(XP_011526042) c3(zinc finger protein 676 isoform X5) c4(320373) c5(320374, 320375, 320376, 320377, 320378) c6( ) c7(hz); #c1(ZNF678) c2(NP_848644) c3(zinc finger protein 678) c4(254048, 254049, 254050) c5(254051, 254052, 254053, 254054, 254055, 254056, 254057, 254058, 254059, 254060, 254061, 254062, 254063, 254064, 254065, 254066, 254067, 254068) c6( ) c7(xb); #c1(ZNF683) c2(XP_011539498) c3(zinc finger protein 683 isoform X2) c4(337698) c5(337699) c6( ) c7(NI, ae, ad); #c1(ZNF687) c2(NP_001291693) c3(zinc finger protein 687) c4(116342) c5(116343, 116344, 116345, 116346, 116347, 116348, 116349, 116350, 116351, 116352, 116353, 116354, 116355, 116356, 116357, 116358, 116359, 116360, 116361) c6( ) c7(bt); #c1(ZNF687) c2(XP_005245423) c3(zinc finger protein 687 isoform X2) c4(266565, 266566) c5(266567, 266568, 266569) c6( ) c7(bt); #c1(ZNF689) c2(NP_612456) c3(zinc finger protein 689) c4(236535, 236536) c5(236537, 236538, 236539, 236540, 236541, 236542, 236543, 236544, 236545, 236546, 236547, 236548, 236549) c6( ) c7(iR, aq, B, eZ); #c1(ZNF7) c2(NP_001269725) c3(zinc finger protein 7 isoform 3) c4(102720) c5(102721, 102722, 102723, 102724, 102725, 102726) c6( ) c7(bL, pk); #c1(ZNF7) c2(XP_006716719) c3(zinc finger protein 7 isoform X2) c4( ) c5(290440, 290441, 290442, 290443, 290444) c6( ) c7(bL, pk); #c1(ZNF7) c2(XP_011515593) c3(zinc finger protein 7 isoform X4) c4( ) c5(308421) c6( ) c7(bL, pk); #c1(ZNF7) c2(XP_011515594) c3(zinc finger protein 7 isoform X3) c4(308422) c5(308423, 308424, 308425, 308426, 308427, 308428) c6( ) c7(bL, pk); #c1(ZNF7) c2(XP_011515596) c3(zinc finger protein 7 isoform X1) c4(308429, 308430) c5(308431, 308432, 308433, 308434, 308435, 308436, 308437, 308438, 308439, 308440, 308441, 308442) c6( ) c7(bL, pk); #c1(ZNF703) c2(NP_079345) c3(zinc finger protein 703) c4(217769, 217770) c5(217771, 217772, 217773, 217774, 217775, 217776, 217777, 217778, 217779, 217780, 217781, 217782, 217783) c6( ) c7(aW, T, aT); #c1(ZNF704) c2(NP_001028895) c3(zinc finger protein 704) c4( ) c5(30697, 30698, 30699, 30700, 30701) c6( ) c7(iU, bt, c); #c1(ZNF704) c2(XP_005251336) c3(zinc finger protein 704 isoform X1) c4(270882) c5(270883, 270884) c6( ) c7(iU, bt, c); #c1(ZNF704) c2(XP_006716527) c3(zinc finger protein 704 isoform X2) c4(290371) c5(290372, 290373, 290374, 290375, 290376) c6( ) c7(iU, bt, c); #c1(ZNF706) c2(NP_001254638) c3(zinc finger protein 706) c4(94743, 94744) c5(94745, 94746, 94747, 94748, 94749, 94750, 94751, 94752, 94753, 94754, 94755, 94756, 94757, 94758, 94759, 94760, 94761, 94762, 94763, 94764, 94765, 94766, 94767, 94768, 94769, 94770, 94771, 94772, 94773, 94774, 94775) c6( ) c7(cd); #c1(ZNF711) c2(NP_068838) c3(zinc finger protein 711) c4(210861) c5(210862, 210863, 210864, 210865, 210866, 210867, 210868, 210869, 210870, 210871, 210872) c6( ) c7(nf, cVM); #c1(ZNF711) c2(XP_005262245) c3(zinc finger protein 711 isoform X3) c4(277793, 277794) c5(277795, 277796, 277797, 277798, 277799, 277800, 277801, 277802, 277803, 277804, 277805, 277806) c6( ) c7(nf, cVM); #c1(ZNF716) c2(NP_001152751) c3(zinc finger protein 716) c4(60129, 60130) c5(60131, 60132, 60133, 60134, 60135, 60136, 60137, 60138, 60139, 60140, 60141, 60142) c6( ) c7(fq, Ea); #c1(ZNF717) c2(NP_001277137) c3(zinc finger protein 717 isoform a) c4( ) c5(109114, 109115, 109116, 109117, 109118) c6( ) c7(aur, Kc, oe, dY, aj); #c1(ZNF717) c2(XP_006712975) c3(zinc finger protein 717 isoform X5) c4(286929) c5(286930, 286931, 286932, 286933, 286934, 286935) c6( ) c7(aur, Kc, oe, dY, aj); #c1(ZNF717) c2(XP_011531546) c3(zinc finger protein 717 isoform X2) c4(327671) c5( ) c6( ) c7(aur, Kc, oe, dY, aj); #c1(ZNF717) c2(XP_011531547) c3(zinc finger protein 717 isoform X3) c4( ) c5(327672) c6( ) c7(aur, Kc, oe, dY, aj); #c1(ZNF717) c2(XP_011531551) c3(zinc finger protein 717 isoform X8) c4( ) c5(327673) c6( ) c7(aur, Kc, oe, dY, aj); #c1(ZNF74) c2(NP_001243453) c3(zinc finger protein 74 isoform a) c4(90826, 90827, 90828) c5(90829, 90830, 90831, 90832, 90833, 90834, 90835, 90836, 90837, 90838, 90839, 90840, 90841, 90842, 90843, 90844, 90845, 90846, 90847, 90848, 90849, 90850, 90851) c6( ) c7(RX); #c1(ZNF746) c2(NP_001156946) c3(zinc finger protein 746 isoform 1) c4(62040) c5(62041, 62042, 62043, 62044, 62045, 62046) c6( ) c7(K, c); #c1(ZNF746) c2(NP_689770) c3(zinc finger protein 746 isoform 2) c4(243916) c5(243917, 243918, 243919, 243920, 243921, 243922) c6( ) c7(K, c); #c1(ZNF750) c2(NP_078978) c3(zinc finger protein 750) c4(216374, 216375) c5(216376, 216377, 216378, 216379, 216380, 216381, 216382, 216383, 216384, 216385, 216386, 216387) c6( ) c7(aOR, cVN, azP, cr); #c1(ZNF76) c2(NP_001278961) c3(zinc finger protein 76 isoform 2) c4(111117) c5(111118, 111119, 111120, 111121, 111122, 111123, 111124, 111125, 111126, 111127, 111128, 111129, 111130, 111131, 111132, 111133, 111134, 111135, 111136, 111137, 111138, 111139, 111140, 111141) c6( ) c7(de); #c1(ZNF76) c2(NP_003418) c3(zinc finger protein 76 isoform 1) c4(138296, 138297, 138298, 138299, 138300) c5(138301, 138302, 138303, 138304, 138305, 138306, 138307, 138308, 138309, 138310, 138311, 138312, 138313) c6( ) c7(oe); #c1(ZNF76) c2(XP_011513157) c3(zinc finger protein 76 isoform X7) c4( ) c5(305013) c6( ) c7(oe); #c1(ZNF76) c2(XP_011513158) c3(zinc finger protein 76 isoform X8) c4( ) c5(305014) c6( ) c7(de); #c1(ZNF763) c2(NP_001012771) c3(zinc finger protein 763) c4(25296, 25297, 25298, 25299) c5(25300, 25301, 25302, 25303, 25304, 25305, 25306, 25307, 25308, 25309, 25310, 25311, 25312, 25313, 25314, 25315, 25316, 25317, 25318, 25319, 25320, 25321, 25322, 25323) c6( ) c7(T, dA, WT, ci, PJ, eW, hX, h, BZ, K, cOR, fb, avQ, ak, anR, d, CT, lo, as, pb, ck, Qi, iO, vW, c, iT, eB, U, bC, bt, xg, B); #c1(ZNF764) c2(NP_001166150) c3(zinc finger protein 764 isoform 2) c4(68346) c5(68347, 68348, 68349, 68350, 68351, 68352) c6( ) c7(pv); #c1(ZNF764) c2(NP_219363) c3(zinc finger protein 764 isoform 1) c4(228566) c5(228567, 228568, 228569, 228570, 228571, 228572, 228573) c6( ) c7(pv); #c1(ZNF765) c2(XP_006723529) c3(zinc finger protein 765 isoform X1) c4(295234) c5(295235, 295236, 295237, 295238, 295239, 295240, 295241) c6( ) c7(fr); #c1(ZNF765) c2(XP_006723530) c3(zinc finger protein 765 isoform X2) c4(295242) c5(295243, 295244, 295245, 295246, 295247) c6( ) c7(fr); #c1(ZNF774) c2(NP_001004309) c3(zinc finger protein 774) c4(20525, 20526) c5(20527, 20528, 20529, 20530, 20531, 20532, 20533, 20534, 20535, 20536, 20537, 20538) c6( ) c7(y); #c1(ZNF776) c2(NP_775903) c3(zinc finger protein 776) c4(250647, 250648, 250649) c5(250650, 250651, 250652, 250653, 250654, 250655) c6( ) c7(fq); #c1(ZNF778) c2(NP_001188336) c3(zinc finger protein 778 isoform 1) c4( ) c5(79085, 79086, 79087, 79088, 79089) c6( ) c7(bN); #c1(ZNF778) c2(NP_872337) c3(zinc finger protein 778 isoform 2) c4(256138) c5(256139, 256140, 256141, 256142, 256143, 256144) c6( ) c7(bN); #c1(ZNF778) c2(XP_011521241) c3(zinc finger protein 778 isoform X2) c4(316153) c5(316154) c6( ) c7(bN); #c1(ZNF784) c2(NP_976308) c3(zinc finger protein 784) c4(263095) c5(263096, 263097, 263098, 263099, 263100, 263101, 263102) c6( ) c7(aw); #c1(ZNF79) c2(NP_001273625) c3(zinc finger protein 79 isoform b) c4( ) c5(105495, 105496, 105497, 105498, 105499) c6( ) c7(ac); #c1(ZNF79) c2(NP_009066) c3(zinc finger protein 79 isoform a) c4(170551, 170552) c5(170553, 170554, 170555, 170556, 170557, 170558, 170559, 170560) c6( ) c7(ac); #c1(ZNF80) c2(NP_009067) c3(zinc finger protein 80) c4(170561) c5(170562, 170563, 170564, 170565, 170566, 170567, 170568, 170569, 170570, 170571, 170572) c6( ) c7(Z, bS, ct); #c1(ZNF800) c2(XP_005250238) c3(zinc finger protein 800 isoform X1) c4( ) c5(270221, 270222, 270223, 270224, 270225, 270226, 270227, 270228, 270229, 270230, 270231, 270232, 270233, 270234, 270235, 270236, 270237, 270238, 270239, 270240, 270241, 270242, 270243, 270244, 270245, 270246, 270247, 270248, 270249, 270250, 270251, 270252) c6( ) c7(bL); #c1(ZNF800) c2(XP_011514161) c3(zinc finger protein 800 isoform X2) c4(306739, 306740, 306741, 306742) c5( ) c6( ) c7(bL); #c1(ZNF804A) c2(NP_919226) c3(zinc finger protein 804A) c4(258261) c5(258262, 258263, 258264, 258265, 258266, 258267) c6( ) c7(mU, cA, rz, mW, mZ, ha, mS, gl, mY, he, bK, uJ, yA, fr, mV, lB, mT, bN, mX); #c1(ZNF804B) c2(NP_857597) c3(zinc finger protein 804B) c4(255276) c5(255277, 255278, 255279, 255280, 255281, 255282) c6( ) c7(gT, fq, ac, iX); #c1(ZNF81) c2(XP_011542204) c3(zinc finger protein 81 isoform X1) c4(340683, 340684, 340685, 340686) c5(340687, 340688, 340689, 340690, 340691, 340692, 340693, 340694, 340695, 340696, 340697, 340698, 340699, 340700, 340701, 340702, 340703, 340704, 340705, 340706, 340707, 340708, 340709, 340710, 340711, 340712, 340713, 340714, 340715, 340716, 340717, 340718) c6( ) c7(nf, cVO, iJ, nA); #c1(ZNF812) c2(XP_011526544) c3(putative zinc finger protein 812 isoform X1) c4( ) c5(320840) c6( ) c7(bK); #c1(ZNF812) c2(XP_011526545) c3(putative zinc finger protein 812 isoform X2) c4(320841) c5(320842, 320843, 320844, 320845, 320846, 320847) c6( ) c7(bk); #c1(ZNF813) c2(NP_001004301) c3(zinc finger protein 813) c4(20518) c5(20519, 20520, 20521, 20522, 20523, 20524) c6( ) c7(j); #c1(ZNF816) c2(NP_001189386) c3(zinc finger protein 816) c4(79319, 79320) c5(79321, 79322, 79323, 79324, 79325, 79326, 79327, 79328, 79329, 79330, 79331, 79332, 79333, 79334, 79335, 79336) c6( ) c7(cr, Jr); #c1(ZNF823) c2(NP_001073962) c3(zinc finger protein 823 isoform 1) c4(36707, 36708, 36709) c5(36710, 36711, 36712, 36713, 36714, 36715, 36716) c6( ) c7(d, aW, oe, dh, aT, aj, v, ei, y, J, hX); #c1(ZNF823) c2(NP_001284539) c3(zinc finger protein 823 isoform 3) c4( ) c5(112002, 112003, 112004, 112005, 112006) c6( ) c7(d, aW, oe, dh, aT, aj, v, ei, y, J, hX); #c1(ZNF823) c2(NP_059977) c3(zinc finger protein 823 isoform 2) c4( ) c5(193430, 193431, 193432, 193433, 193434, 193435) c6( ) c7(d, aW, oe, dh, aT, aj, v, ei, y, J, hX); #c1(ZNF827) c2(NP_849157) c3(zinc finger protein 827) c4(254320) c5(254321, 254322, 254323, 254324, 254325) c6( ) c7(oe); #c1(ZNF827) c2(XP_011529935) c3(zinc finger protein 827 isoform X2) c4( ) c5(325739) c6( ) c7(oe); #c1(ZNF827) c2(XP_011529936) c3(zinc finger protein 827 isoform X3) c4( ) c5(325740, 325741, 325742) c6( ) c7(oe); #c1(ZNF829) c2(NP_001165450) c3(zinc finger protein 829 isoform 1) c4(67749) c5(67750, 67751) c6( ) c7(q, r); #c1(ZNF829) c2(XP_011525235) c3(zinc finger protein 829 isoform X1) c4( ) c5(319704, 319705, 319706, 319707, 319708, 319709, 319710, 319711) c6( ) c7(q, r); #c1(ZNF831) c2(NP_848552) c3(zinc finger protein 831) c4(253834) c5(253835, 253836, 253837, 253838, 253839, 253840, 253841) c6( ) c7(Y, aC, ac, Be); #c1(ZNF91) c2(NP_001287880) c3(zinc finger protein 91 isoform 2) c4(113111) c5(113112, 113113, 113114, 113115, 113116, 113117) c6( ) c7(wu, wm); #c1(ZNF91) c2(NP_003421) c3(zinc finger protein 91 isoform 1) c4(138314) c5(138315, 138316, 138317, 138318, 138319, 138320, 138321) c6( ) c7(wu, wm); #c1(ZNF91) c2(XP_006722943) c3(zinc finger protein 91 isoform X2) c4( ) c5(294956) c6( ) c7(wu, wm); #c1(ZNF92) c2(NP_001274461) c3(zinc finger protein 92 isoform 3) c4(106555) c5(106556, 106557, 106558, 106559, 106560, 106561, 106562) c6( ) c7(ae, ad); #c1(ZNF92) c2(NP_009070) c3(zinc finger protein 92 isoform 1) c4( ) c5(170573, 170574, 170575, 170576, 170577) c6( ) c7(ae, ad); #c1(ZNF92) c2(NP_689839) c3(zinc finger protein 92 isoform 2) c4(244122, 244123) c5(244124, 244125, 244126, 244127, 244128, 244129, 244130) c6( ) c7(ae, ad); #c1(ZNF93) c2(NP_112495) c3(zinc finger protein 93) c4(221165, 221166) c5(221167, 221168, 221169, 221170, 221171, 221172, 221173, 221174, 221175, 221176, 221177, 221178, 221179) c6( ) c7(T, dx); #c1(ZNF93) c2(XP_011526641) c3(zinc finger protein 93 isoform X1) c4(320942) c5(320943, 320944, 320945, 320946, 320947, 320948) c6( ) c7(T, dx); #c1(ZNF98) c2(NP_001092096) c3(zinc finger protein 98) c4(39331, 39332) c5(39333, 39334, 39335, 39336, 39337, 39338, 39339) c6( ) c7(ab, aw); #c1(ZNFX1) c2(NP_066363) c3(NFX1-type zinc finger-containing protein 1) c4(207225, 207226, 207227, 207228, 207229, 207230) c5(207231, 207232, 207233, 207234, 207235, 207236, 207237, 207238, 207239, 207240, 207241, 207242, 207243, 207244, 207245, 207246, 207247, 207248, 207249, 207250, 207251, 207252, 207253, 207254, 207255, 207256, 207257, 207258, 207259, 207260, 207261, 207262, 207263, 207264) c6( ) c7(ad); #c1(ZNFX1) c2(XP_011527226) c3(NFX1-type zinc finger-containing protein 1 isoform X2) c4( ) c5(321773, 321774, 321775, 321776, 321777, 321778) c6( ) c7(ad); #c1(ZNHIT2) c2(NP_055020) c3(zinc finger HIT domain-containing protein 2) c4(178327) c5(178328, 178329, 178330, 178331, 178332, (78333) c6( ) c7(nJ, Y); #c1(ZNHIT3) c2(NP_004764) c3(zinc finger HIT domain-containing protein 3 isoform 1) c4(149401) c5(149402, 149403, 149404, 149405, 149406, 149407) c6( ) c7(go, ey, k); #c1(ZNRD1) c2(NP_001265715) c3(DNA-directed RNA polymerase 1 subunit RPA12) c4(99190) c5(99191, 99192, 99193, 99194, 99195, 99196, 99197, 99198, 99199, 99200, 99201, 99202, 99203, 99204, 99205, 99206, 99207, 99208, 99209, 99210, 99211, 99212, 99213, 99214, 99215, 99216, 99217, 99218, 99219, 99220, 99221, 99222, 99223, 99224, 99225, 99226, 99227, 99228, 99229, 99230, 99231, 99232, 99233, 99234, 99235, 99236, 99237, 99238, 99239, 99240, 99241, 99242, 99243, 99244, 99245, 99246, 99247, 99248, 99249, 99250, 99251, 99252, 99253, 99254, 99255, 99256, 99257, 99258, 99259, 99260, 99261, 99262, 99263, 99264, 99265, 99266, 99267, 99268, 99269, 99270, 99271, 99272, 99273, 99274, 99275, 99276, 99277, 99278, 99279, 99280, 99281, 99282, 99283, 99284, 99285, 99286, 99287, 99288, 99289, 99290, 99291, 99292, 99293, 99294, 99295, 99296, 99297, 99298, 99299, 99300) c6( ) c7(ck, bM, iQ, aDy, aD, ht, qh, eZ, aA, h, aF, hu, g, Sm); #c1(ZNRF3) c2(NP_001193927) c3(E3 ubiquitin-protein ligase ZNRF3 isoform 1 precursor) c4(83195, 83196) c5(83197, 83198, 83199, 83200, 83201, 83202, 83203, 83204, 83205, 83206, 83207, 83208, 83209, 83210, 83211, 83212, 83213, 83214, 83215, 83216, 83217, 83218, 83219, 83220, 83221) c6( ) c7(iv, T, aC); #c1(ZNRF3) c2(XP_011528740) c3(E3 ubiquitin-protein ligase ZNRF3 isoform X3) c4(324052) c5( ) c6( ) c7(iv, T, aC); #c1(ZNRF3) c2(XP_011528741) c3(E3 ubiquitin-protein ligase ZNRF3 isoform X4) c4( ) c5(324053) c6( ) c7(iv, T, aC); #c1(ZP1) c2(NP_997224) c3(zona pellucida sperm-binding protein 1 precursor) c4(265203) c5(265204, 265205, 265206, 265207, 265208, 265209, 265210, 265211, 265212) c6( ) c7(qq); #c1(ZP1) c2(XP_011543155) c3(zona pellucida sperm-binding protein 1 isoform X1) c4(341983) c5(341984, 341985, 341986, 341987, 341988) c6( ) c7(qq); #c1(ZP4) c2(NP_067009) c3(zona pellucida sperm-binding protein 4 preproprotein) c4(208302) c5(208303, 208304, 208305, 208306, 208307, 208308, 208309) c6( ) c7(V, pZ, cz); #c1(ZPBP2) c2(NP_942141) c3(zona pellucida-binding protein 2 isoform 1 precursor) c4(260545) c5(260546, 260547, 260548, 260549, 260550, 260551) c6( ) c7(bM, nH, A, eZ, iV, y); #c1(ZPBP2) c2(NP_955353) c3(zona pellucida-binding protein 2 isoform 2 precursor) c4(261531) c5(261532, 261533, 261534, 261535, 261536, 261537) c6( ) c7(bM, nH, A, eZ, iV, y); #c1(ZPLD1) c2(NP_778226) c3(zona pellucida-like domain-containing protein 1 precursor) c4(251419) c5(251420, 251421, 251422, 251423, 251424, 251425, 251426, 251427) c6( ) c7(bL, ac, aw, v, j, cd); #c1(ZPLD1) c2(XP_011510713) c3(zona pellucida-like domain-containing protein 1 isoform X1) c4(301148) c5( ) c6( ) c7(bL, ac, aw, v, j, cd); #c1(ZPLD1) c2(XP_011510714) c3(zona pellucida-like domain-containing protein 1 isoform X2) c4( ) c5(301149, 301150, 301151, 301152, 301153, 301154, 301155, 301156) c6( ) c7(bL, ac, aw, v, j, cd); #c1(ZPR1) c2(NP_003895) c3(zinc finger protein ZPR1) c4(141932) c5(141933, 141934, 141935, 141936, 141937, 141938, 141939) c6( ) c7(cO, om, e, kf, j, ae, ad); #c1(ZRANB3) c2(NP_001273497) c3(DNA annealing helicase and endonuclease ZRANB3 isoform 2) c4(105059, 105060, 105061, 105062, 105063, 105064) c5(105065, 105066, 105067, 105068, 105069, 105070, 105071, 105072, 105073, 105074, 105075, 105076, 105077, 105078, 105079, 105080, 105081, 105082, 105083, 105084, 105085, 105086, 105087, 105088, 105089, 105090, 105091, 105092, 105093, 105094) c6( ) c7(cm, K, ek); #c1(ZRANB3) c2(NP_115519) c3(DNA annealing helicase and endonuclease ZRANB3 isoform 1) c4(223056) c5(223057, 223058, 223059, 223060, 223061, 223062, 223063) c6( ) c7(cm, K, ek); #c1(ZRANB3) c2(XP_011510269) c3(DNA annealing helicase and endonuclease ZRANB3 isoform X4) c4( ) c5(300863, 300864, 300865, 300866, 300867) c6( ) c7(cm, K, ek); #c1(ZRSR2) c2(NP_005080) c3(U2 small nuclear ribonucleoprotein auxiliary factor 35 kDa subunit-related protein 2) c4(152721, 152722) c5(152723) c6( ) c7(gV, T, bs, bw, bt, af, br); #c1(ZRSR2) c2(XP_006724582) c3(U2 small nuclear ribonucleoprotein auxiliary factor 35 kDa subunit-related protein 2 isoform X2) c4(296094) c5( ) c6( ) c7(gV, T, bs, bw, bt, af, br); #c1(ZRSR2) c2(XP_011543891) c3(U2 small nuclear ribonucleoprotein auxiliary factor 35 kDa subunit-related protein 2 isoform X1) c4( ) c5(343063, 343064, 343065, 343066, 343067, 343068) c6( ) c7(gV, T, bs, bw, bt, af, br); #c1(ZRSR2) c2(XP_011543892) c3(U2 small nuclear ribonucleoprotein auxiliary factor 35 kDa subunit-related protein 2 isoform X3) c4( ) c5(343069, 343070, 343071, 343072, 343073, 343074, 343075) c6( ) c7(gV, T, bs, bw, bt, af, br); #c1(ZSCAN18) c2(NP_001139014) c3(zinc finger and SCAN domain-containing protein 18 isoform 1) c4(58598) c5(58599, 58600, 58601, 58602, 58603, 58604, 58605, 58606, 58607, 58608, 58609) c6( ) c7(fr, jz, q, gX, r); #c1(ZSCAN18) c2(NP_001139016) c3(zinc finger and SCAN domain-containing protein 18 isoform 2) c4(58610) c5(58611, 58612, 58613, 58614, 58615, 58616) c6( ) c7(fr, jz, q, gX, r); #c1(ZSCAN18) c2(XP_005259231) c3(zinc finger and SCAN domain-containing protein 18 isoform X1) c4(276009) c5(276010, 276011, 276012, 276013, 276014) c6( ) c7(fr, jz, q, gX, r); #c1(ZSCAN22) c2(NP_862829) c3(zinc finger and SCAN domain-containing protein 22) c4(255804) c5(255805, 255806, 255807, 255808, 255809, 255810) c6( ) c7(aq); #c1(ZSCAN26) c2(NP_001104509) c3(zinc finger and SCAN domain-containing protein 26 isoform b) c4( ) c5(42152) c6( ) c7(eZ); #c1(ZSCAN31) c2(NP_001230173) c3(zinc finger and SCAN domain-containing protein 31 isoform 2) c4( ) c5(86041, 86042, 86043, 86044, 86045) c6( ) c7(fq, eZ); #c1(ZSCAN31) c2(XP_011513115) c3(zinc finger and SCAN domain-containing protein 31 isoform X1) c4(304833, 304834, 304835, 304836, 304837, 304838, 304839, 304840, 304841, 304842) c5(304843, 304844, 304845, 304846, 304847, 304848, 304849, 304850, 304851, 304852, 304853, 304854, 304855, 304856, 304857, 304858, 304859, 304860, 304861, 304862, 304863, 304864, 304865, 304866, 304867, 304868, 304869, 304870, 304871, 304872, 304873, 304874, 304875, 304876, 304877, 304878, 304879, 304880, 304881, 304882, 304883, 304884, 304885, 304886, 304887, 304888, 304889, 304890, 304891, 304892, 304893, 304894, 304895, 304896, 304897, 304898, 304899, 304900, 304901, 304902, 304903, 304904, 304905, 304906, 304907, 304908) c6( ) c7(fq, eZ); #c1(ZSCAN32) c2(NP_001271458) c3(zinc finger and SCAN domain-containing protein 32 isoform c) c4( ) c5(103980, 103981, 103982, 103983, 103984) c6( ) c7(oe); #c1(ZSCAN32) c2(XP_005255461) c3(zinc finger and SCAN domain-containing protein 32 isoform X5) c4( ) c5(273672) c6( ) c7(oe); #c1(ZSCAN32) c2(XP_011520857) c3(zinc finger and SCAN domain-containing protein 32 isoform X1) c4(315839, 315840, 315841, 315842) c5(315843, 315844, 315845, 315846, 315847, 315848, 315849, 315850, 315851, 315852, 315853, 315854, 315855, 315856, 315857, 315858, 315859, 315860, 315861) c6( ) c7(De); #c1(ZSCAN32) c2(XP_011520859) c3(zinc finger and SCAN domain-containing protein 32 isoform X2) c4(315862) c5(315863, 315864, 315865, 315866, 315867, 315868, 315869, 315870, 315871, 315872) c6( ) c7(oe); #c1(ZSCAN9) c2(NP_001186408) c3(zinc finger and SCAN domain-containing protein 9 isoform 1) c4( ) c5(77625, 77626, 77627, 77628, 77629) c6( ) c7(fq, eZ); #c1(ZSCAN9) c2(NP_006290) c3(zinc finger and SCAN domain-containing protein 9 isoform 2) c4(163718) c5(163719, 163720, 163721, 163722, 163723, 163724, 163725, 163726, 163727, 163728, 163729, 163730, 163731) c6( ) c7(fq, eZ); #c1(ZSCAN9) c2(XP_011513178) c3(zinc finger and SCAN domain-containing protein 9 isoform X2) c4(305108) c5(305109) c6( ) c7(fq, eZ); #c1(ZSCAN9) c2(XP_011513180) c3(zinc finger and SCAN domain-containing protein 9 isoform X4) c4(305110) c5(305111, 305112, 305113, 305114) c6( ) c7(fq, eZ); #c1(ZSWIM2) c2(NP_872327) c3(E3 ubiquitin-protein ligase ZSWIM2) c4(256125) c5(256126, 256127, 256128, 256129, 256130, 256131) c6( ) c7(ac); #c1(ZSWIM6) c2(NP_065979) c3(zinc finger SWIM domain-containing protein 6) c4(206591) c5(206592, 206593, 206594, 206595, 206596, 206597) c6( ) c7(cVP, atb); #c1(ZW10) c2(NP_004715) c3(centromere/kinetochore protein zw10 homolog) c4(149022, 149023) c5(149024, 149025, 149026, 149027, 149028, 149029, 149030, 149031, 149032, 149033, 149034, 149035) c6( ) c7(eF); #c1(ZWILCH) c2(NP_001274752) c3(protein zwilch homolog isoform 2) c4( ) c5(106765, 106766, 106767, 106768, 106769, 106770, 106771, 106772) c6( ) c7(aW, aT); #c1(ZWILCH) c2(NP_060445) c3(protein zwilch homolog isoform 1) c4(195976, 195977) c5(195978, 195979, 195980, 195981, 195982, 195983, 195984, 195985) c6( ) c7(aW, aT); #c1(ZWINT) c2(NP_001005413) c3(ZW10 interactor isoform b) c4(21654) c5(21655, 21656, 21657, 21658, 21659, 21660, 21661, 21662, 21663, 21664, 21665, 21666) c6( ) c7(aW, aT, ey, an, ae, af, ad); #c1(ZWINT) c2(NP_008988) c3(ZW10 interactor isoform a) c4(170092, 170093, 170094) c5(170095, 170096, 170097, 170098, 170099, 170100, 170101, 170102, 170103, 170104, 170105, 170106, 170107, 170108, 170109, 170110) c6( ) c7(aW, aT, ey, an, ae, af, ad); #c1(ZYX) c2(NP_001010972) c3(zyxin) c4(24360, 24361, 24362) c5(24363, 24364, 24365, 24366, 24367, 24368, 24369, 24370, 24371, 24372, 24373, 24374, 24375, 24376, 24377, 24378, 24379, 24380) c6( ) c7(ck, T, bE, Y, ff, hj, cR, bB, Gn, K, en, rA, bz, B, dx); #c1(ZYX) c2(XP_011514869) c3(zyxin isoform X1) c4( ) c5(307617) c6( ) c7(ck, T, bE, Y, ff, hj, cR, bB, Gn, K, en, rA, bz, B, dx); #c1(ZYX) c2(XP_011514871) c3(zyxin isoform X3) c4(307618, 307619) c5(307620, 307621, 307622, 307623, 307624, 307625, 307626, 307627, 307628, 307629, 307630, 307631) c6( ) c7(ck, T, bE, Y, ff, hj, cR, bB, Gn, K, en, rA, bz, B, dx); #c1(ZZEF1) c2(NP_055928) c3(zinc finger ZZ-type and EF-hand domain-containing protein 1) c4(184490) c5(184491, 184492, 184493, 184494, 184495, 184496) c6( ) c7(T); #c1(ZZEF1) c2(XP_011522061) c3(zinc finger ZZ-type and EF-hand domain-containing protein 1 isoform X6) c4( ) c5(316717) c6( ) c7(T); #c1(ZZZ3) c2(XP_011539507) c3(ZZ-type zinc finger-containing protein 3 isoform X3) c4(337706) c5(337707, 337708, 337709, 337710, 337711, 337712, 337713) c6( ) c7(v);

TABLE C
Abbreviation Dictionary for the “Related disease, disorder or condition” corresponding to feature “c7”, whereby
each Related disease, disorder or condition is depicted with a specific abbreviation and its written form, i.e.
prostate cancer corresponds to the abbreviation “A” and vice versa.
Formula: Related disease, disorder or condition = Abbreviation;

type 1 diabetes mellitus = A; stomach cancer = aA; lung disease = aa; cerebral vasospasm = Aa; renal phosphaturia = AA;
ascending colon cancer = aAa; apolipoprotein c-ii deficiency = aaa; chlamydophila infections = aaA; high grade dysplastic
nodule = aAA; lupus-like syndrome = aAB; mental retardation and microcephaly with pontine and cerebellar hypoplasia =
aAb; postoperative confusion = aaB; chylomicronemia syndrome = aab; low grade dysplastic nodule = aAC; fg syndrome 4 =
aAc; myotonic muscular dystrophy = aac; carbon monoxide poisoning = aaC; x-linked infantile nystagmus = aAd; deficiency
of triacylglycerol lipase = aad; anthracosis = aAD; warfarin sensitivity = aaD; germinoma = aAE; fg syndrome = aAe; steatorrhea =
aae; grn-related frontotemporal dementia = aaE; marden-walker syndrome = aAf; acquired cjd = aaf; apolipoprotein c-iii
deficiency = aaf; supratentorial primitive neuroectodermal tumor = aAF; corticobasal degeneration = aaG; tissue sensitivity =
aAG; fat redistribution = aag; bacterial sepsis = aAg; alzheimer's disease with early onset = aaH; intestinal carcinoma =
aah; cystinosis = aAh; hyperparathyroidism 1 = aAH; amebiasis = aAi; hyperparathyroidism, neonatal severe primary =
aAI; akathisia, drug-induced = aaI; hypotriglyceridaemia = aai; autoimmune lymphoproliferative syndrome, type iia = aAj;
subarachnoid hemorrhage, aneurysmal = aaJ; dermatofibrosarcoma protuberans = aaj; epilepsy, idiopathyic generalized,
susceptibility to, 8 = aAJ; lipoprotein glomerulopathy = aak; spinal osteophytosis = aaK; hypocalcemia, autosomal dominant
1 = aAK; caspase 8 deficiency = aAk; dementia, familial danish = aal; vertebral artery dissection = aaL; hydrosalpinx =
aAl; hyperparathyroidism 3 = aAL; perceptual disorders = aaM; neuroferritinopathy = aAm; pyruvate dehydrogenase e3-binding
protein deficiency = aam; hypercalciuric hypercalcemia = aAM; fragile × tremor ataxia syndrome = aan; disorders of excessive
somnolence = aaN; nephrolithiasis, calcium oxalate = aAN; reoviridae infections = aAn; inclusion body myopathy, sporadic =
aaO; hyperparathyroid = aAO; cerebral amyloid angiopathy, familial = aao; posterior capsule opacification = aAo; idiopathyic
hypercalciuria = aAP; alcohol withdrawal seizures = aap; soft drusen = aaP; spinal cord stroke = aAp; speech-sound disorder =
aaq; neonatal hyperparathyroidism = aAQ; toxicosis = aaQ; erosisve esophagitis = aAq; infiltrating duct carcinoma =
aAr; calcium metabolism disease = aAR; stomatitis, herpetic = aaR; plaque, atherosclerotic = aar; hypocapnia = aAs; depressive
syndrome = aaS; fetal nutrition disorders = aas; lactose intolerance = aAS; mercury poisoning, nervous system = aat;
ebola hemorrhagic fever = aAT; hypersensitive syndrome = aaT; krabbe disease = aAt; glaucomatous retinal degeneration =
aAU; mercury poisoning = aau; vulva cancer = aAu; keratitis, herpetic = aaU; encephalyitozoonosis = aAv; amyotrophyic
lateral sclerosis-parkinsonism/dementia complex 1 = aav; vulvovaginal atrophy = aAV; lupus vasculitis, central nervous
system = aaV; neurobehavioral manifestations = aaw; enteropathyogenic escherichia coli gastrointestinal tract infection =
aAw; cross infection = aaW; recessive dystrophyic epidermaolysis bullosa = aAW; stage, neuroblastoma = aAx; yin deficiency =
aaX; abnormalities, drug-induced = aAX; sensation disorders = aax; amyloidosis, primary cutaneous = aay; terminal ileitis =
aAy; acatalasemia japanese type = aAY; closed head injuries = aaY; renoprival hypertension = aAZ; tumors in children =
aAz; head injuries, closed = aaz; persistent vegetative state = aaZ; keratoconus = ab; nemaline myopathy 3 = Ab; peptic
ulcer disease = aB; familial x-linked hypophosphatemiac vitamin d refractory rickets = AB; intravenous drug abuse = abA;
muscular dystrophy, limb-girdle, type 1c = aBA; cerebral hemorrhage, traumatic = aba; radiotoxicity = aBa; arteriosclerosis
of aorta = abB; deficiency of catalase = aBb; long qt syndrome 9 = aBB; diarrhea, infantile = abb; paramyxoviridae infections =
abc; creatine phosphokinase, elevated serum = aBC; progressive multiple sclerosis = abC; hypocatalasemia = aBc; dementia
in parkinson's disease = abd; embryopathy = aBd; rippling muscle disease, 1 = aBD; forgetting = abD; reproductive tract
infections = aBe; mixed dementia = abe; chronic anxiety = abE; myopathy, distal, tateyama type = aBE; sexual abuse =
abf; soft neurological signs = abF; female genital tract infection = aBf; rippling muscle disease 2 (disorder) = aBF;
progressive dementia = abg; hyperirritability = aBG; post-concussion syndrome = abG; arterial leg ulcer = aBg; mitral
valve endocarditis = aBh; rippling muscle disease = aBH; leprosy--patients = abh; subcortical vascular dementia = abH;
loss of memory ability = abI; executive dysfunction = abi; core binding factor acute myeloid leukemia = aBI; acute ulcer =
aBi; pipecolic acidemia = aBj; anxiety and fear = abj; sarcoma of mesentery = aBJ; prodromal period = abJ; aspergilloma =
aBk; legius syndrome = aBK; visuospatial deficit = abK; osteoporosis risk = abk; aspergillosis = aBl; dysphoric mood =
abl; daytime somnolence = abL; noonan syndrome-like disorder with or without juvenile myelomaonocytic leukemia = aBL;
prodrome = abM; dementia associated with alcoholism = abm; pericarditis = aBm; clubbing of nail = aBM; mobility poor =
abN; refsum disease = aBn; primary hypertrophyic osteoarthropathy = aBN; early onset schizophrenia = abn; post-traumatic
amnesia = abo; cystathionine beta-synthase deficiency = aBO; recurrent genital herpes simplex = abO; allergic bronchopulmonary
aspergillosis = aBo; sensory denervation = abP; acatalasia = aBp; carotid artery, internal, dissection = aBP; cerebrovascular
amyloidosis = abp; severe dementia = abq; homocystinuria, pyridoxine-responsive = aBQ; photosensitive trichothiodystrophy =
aBq; severe depression = abQ; acute nephropathy = abR; variegate porphyria = aBr; intracranial sinus thrombosis = aBR;
mobility limitation = abr; iatrogenic creutzfeldt-jakob disease = abs; clinical depression = abS; gonorrhea = aBs; 46,
xy sex reversal 5 = aBS; congenital dyserythropoietic anemia, type i = aBT; cardiovascular infections = abT; actinomycosis =
aBt; iodine deficiency syndrome = abt; spermatogenic failure 7 = aBu; tetralogy of fallot with pulmonary atresia =
abu; tuberous xanthoma = abU; otitis media, susceptibility to = aBU; disease = abV; mental retardation, autosomal recessive
3 = aBV; lipodystrophy, congenital generalized, type 3 = aBv; frontal dementia = abv; circulatory arrest = abw; pulmonary
hypertension, primary, 3 = aBw; optic neuritis = abW; coach syndrome = aBW; hypothalamic-pituitary-adrenal axis dysfunction =
abx; joubert syndrome 9 = aBX; hypopharyngeal carcinoma = aBx; siderosis = abX; triplegia = aby; mixed oligoastrocytoma =
aBy; nasopharyngitis = abY; meckel syndrome, type 6 = aBY; intracranial atherosclerosis = abz; meckel syndrome = aBZ;
uterine carcinoma = aBz; tic disorder = abZ; cerebrovascular disease = ac; myopathy, actin, congenital, with cores =
Ac; motor disturbances = AC; pancreatic cancer = aC; hennekam syndrome = aCa; hypoplastic left heart syndrome = acA;
intracranial hypertension = aca; allergic conjunctivitis = aCA; ciliary dyskinesia, primary, 17 = aCb; exanthem = acB;
vitamin k deficiency hemorrhagic disease = acb; herpes gestationis = aCB; ciliary dyskinesia, primary, 20 = aCc; schizophreniform
disorder = acc; macular degeneration, age-related, 2 = aCC; leprosy, multibacillary = acC; ciliary dyskinesia, primary,
14 = aCd; leprosy, paucibacillary = acD; recurrent respiratory papillomatosis = aCD; choroid disease = acd; focal segmental
glomerulosclerosis 4, susceptibility to = acE; patau syndrome = ace; ciliary dyskinesia, primary, 15 = aCe; pulmonary
aspergillosis = aCE; familial hematuria = acF; skin symptom = aCF; deafness, autosomal dominant 44 = aCf; complex partial
epilepsy = acf; mandibuloacral dysplasia with type a lipodystrophy = aCg; pseudopelade = acG; paragonimiasis = aCG; communication
disorder = acg; adenomatous goiter = aCh; aids (disease) = aCH; lichen planus follicularis = acH; quadriplegia = ach;
color blindness = aCi; dermatomycosis = aci; extrinsic allergic alveolitis = aCI; phencyclidine abuse = acI; alveolitis =
aCJ; ciliary dyskinesia, primary, 27 = aCj; intracranial vasospasm = acj; cytochrome-c oxidase deficiency disease =
acJ; cerebral amyloid angiopathy, app-related = acK; cadasil = ack; generalized progressive retinal atrophy = aCk; mycobacterium
tuberculosis, susceptibility to = aCK; coronary artery disease, modifier of = aCL; myopathy, centronuclear, 4 = aCl;
ventricular septal defect = acl; alzheimer disease type 4 = acL; neuropathy, painful = aCM; spondylosis = acm; three
m syndrome 1 = aCm; dementia, familial british = acM; ampulla of vater carcinoma = acn; cerebral hemorrhage with amyloidosis,
hereditary, dutch type = acN; three m syndrome 3 = aCn; nephropathy, chronic tubulointerstitial = aCN; alzheimer disease,
familial, 1 = acO; acute recurrent pancreatitis = aCO; 3-m syndrome = aCo; sea-blue histiocyte syndrome = aco; angiomyolipom =
aCp; bacterial peritonitis = aCP; macular retinal edema = acp; alzheimer disease, early-onset, with cerebral amyloid
angiopathy = acP; cerebral amyloid angiopathy, hereditary = acQ; coronary occlusion = aCQ; nominal aphasia = acq; chronic
dermatitis = aCq; chronic ulcer of skin = aCr; holoprosencephalyy = acr; general paresis = acR; cumulative trauma disorders =
aCR; chronic constipation = aCs; carotid artery occlusion = acS; arteriolosclerosis = acs; cancer pain = aCS; morphine
dependencye = aCt; behavior disturbance = acT; asthmatic pulmonary eosinophilia = aCT; progressive supranuclear palsy =
act; phenylketonuria = acU; herpes zoster = acu; cancer angiogenesis = aCU; idiopathyic environmental intolerance =
aCu; muscle amp deaminase deficiency = acV; viral myocarditis = aCV; high grade astrocytic tumor = aCv; pityriasis versicolor =
acv; multiple chemical sensitivity = aCw; alagille syndrome = acw; viral arthritis = aCW; fibrous dysplasia = acW;
osteodystrophy = acX; infection by leishmania braziliensis = aCX; asthma, susceptibility to = aCx; carotid artery dissection =
acx; leishmaniasis, diffuse cutaneous = aCY; granuloma, respiratory tract = aCy; exophthalmos = acy; deficiency of
phosphorylase kinase = acY; otitis = aCZ; galns deficiency = acZ; human immunodeficiency virus type 1, susceptibility
to = aCz; hypopituitarism = acz; baraitser-winter syndrome = AD; congenital myopathy = Ad; prostate cancer = ad; gastritis =
aD; acute miliary tuberculosis = aDa; priapism = adA; sandhoff disease = aDA; cardiac glycogenosis = ada; seronegative
rheumatoid arthritis = aDb; intestinal schistosomiasis = aDB; diabetes insipidus, nephrogenic, x-linked = adB; conduction
system abnormalities = adb; viral meningoencephalyitis = aDc; diabetes insipidus, nephrogenic, autosomal = adC; methemoglobinemia =
adc; nondisjunction = aDC; toxoplasmosis, animal = aDD; congenital nephrogenic diabetes insipidus = adD; digestive
system cancers = aDd; glycogen storage disease vii = add; mucopolysaccharidosis iv = ade; borderline tuberculoid leprosy =
aDe; balance disorder = adE; pregnancy complications, infectious = aDE; lobar pneumonia = aDF; arthritis symptoms =
aDf; neurohypophyseal diabetes insipidus = adF; mccune albright syndrome = adf; lung disease granulomatous = aDg; diabetes
insipidus = adG; late onset asthma = aDG; skin squamous cell carcinoma = adg; gil blood group = adH; renal vasculitis =
aDh; wolff-parkinson-white syndrome = adh; viral respiratory infection = aDH; pseudopseudohypoparathyroidism = adi;
auricular swelling = aDi; dysequilibrium syndrome = adI; pneumonia in children = aDI; collagen-vascular disease = aDJ;
clear cell sarcoma = adj; recurrent optic neuritis = adJ; pelvic inflammatory disease = aDj; myelitis, transverse = adK;
skin damage = aDK; optic nerve glioma = adk; acute myocarditis = aDk; periapical periodontitis = aDl; acute myocardial
ischemia = adL; yusho disease = aDL; danon disease = adl; atrioventricular block = adm; posterior uveitis = aDM; bilateral
optic neuritis = adM; dengue hemorrhagic fever = aDm; hyponatremic = adN; orchitis = aDn; adenine phosphoribosyltransferase
deficiency = adn; boutonneuse fever = aDN; granulomatous angiitis = aDo; spastic ataxia charlevoix-saguenay type = ado;
obstructive hydrocephalyus = adO; yellow fever = aDO; myelitis = adP; nonspecific interstitial pneumonia = aDP; early-onset
ataxia with oculomotor apraxia and hypoalbuminemia = adp; bronchiolitis = aDp; endocervicitis = aDq; diffuse palmoplantar
keratoderma, bothnian type = adQ; ataxia telangiectasisa like disorder = adq; idiopathyic osteoarthritis = aDQ; marinesco-sjogren
syndrome = adr; degenerative joint disease involving multiple joints = aDR; varicose veinss = aDr; morphologically altered
structure = adR; early onset cerebellar ataxia = ads; cerebral cavernous malformations 2 = aDS; cutaneous leishmaniasis =
aDs; keratitis-ichthyosis-deafness syndrome = adS; mucocutaneous leishmaniasis = aDt; perivascular tumor = adT; cross-dependencye =
adt; richter's syndrome = aDT; acute heart failure = adU; pulmonary eosinophilia = aDu; limb-girdle muscular dystrophy,
type 2b = adu; inflammatory cardiomyopathy = aDU; edema, cardiac = adv; friedreich ataxia 1 = adV; asymptomatic multiple
myeloma = aDV; pancreatic cystadenoma = aDv; congenital dyserythropoietic anemia = adw; esophageal disease = aDW; 46,
xy disorders of sex development = adW; t-lymphocytopenia = aDw; retinal veins occlusion = adx; myasthenia = aDx; coxsackievirus
infections = aDX; congenital adrenal hyperplasia due to 21 hydroxylase deficiency = adX; gastrooesophageal cancer = aDY;
hypospadias 1, x-linked = adY; leishmaniasis = ady; aspirin-sensitive asthma with nasal polyps = aDy; epithelial hyperplasia =
aDZ; 21-hydroxylase deficiency = adZ; visceral leishmaniasis = adz; rocio virus encephalyitis = aDz; muscle degeneration =
Ae; prostate carcinoma = ae; pancreatic carcinoma = aE; qualitative abnormality of granulocyte = AE; mantle lymphoma =
aEa; diabetes mellitus, insulin-dependent, 22 = aEA; reactive thrombocytosis = aeA; bulbospinal neuronopathy, x-linked
recessive = aea; primary graft dysfunction = aEB; gonadal dysgenesis, 45,x = aeB; stage, kaposi's sarcoma = aEb; heredodegenerative
disorders, nervous system = aeb; hiv disease progression = aEC; testicular dysgenesis syndrome = aeC; male hypogonadism =
aec; endometrial sarcoma = aEc; malignant neoplasm of male breast = aed; hernia congenital = aeD; maladjustment = aEd;
substance abuse, intravenous = aED; diseases or conditions = aeE; infertility tubal factor = aEE; high grade endometrial
stromal sarcoma = aEe; acne vulgaris = aee; hiv-1 infection = aEF; supernumerary structure = aeF; endometrial stromal
sarcoma, high grade = aEf; spinal diseases = aef; transsexualism = aeg; asymptomatic human immunodeficiency virus infection =
aEG; palisaded myofibroblastoma = aEg; bilateral inguinal hernia = aeG; cecal benign neoplasm = aEh; intratubular malignant
germ cells = aeH; morning stiffness (>1 hour) = aEH; phenotypic heterogeneity = aeh; laryngeal disease = aEi; neoplasms,
hormone-dependent = aei; infections specific to perinatal period = aEI; disorder of neutrophyils = aeI; ocular surface
disease = aEJ; large prostate = aej; vulva carcinoma = aEj; endocrine glands--cancer = aeJ; summer-type hypersensitivity
pneumoniatis = aeK; 46, xx testicular disorders of sex development = aek; flaviviral diseases = aEK; adenomayoma = aEk;
verrucous carcinoma = aEl; 5-alpha reductase deficiency = ael; dysplastic nodule = aeL; biliary stricture = aEL; endometrial
stromal tumor = aEm; end stage aids = aEM; lower motor neuron disease = aem; alopecia = aeM; motor symptoms = aen; prostate
malignant phyllodes tumor = aeN; primary hiv infection = aEN; anus neoplasm = aEn; atypical polypoid adenomayoma = aEo;
peripheral nervous system neoplasm = aeO; chromosome 11p deletion syndrome = aeo; upper urinary tract infection = aEO;
hypertrophyic pyloric stenosis = aeP; poorly differentiated carcinoma = aep; bubonic plague = aEP; malignant ciliary
body melanoma = aEp; female reproductive system disease = aEQ; gynecomastia = aeQ; senile angioma = aEq; congenital malformation
of genital organs = aeq; subfertility, male = aer; neurofibroma = aeR; thoracic injuries = aEr; natural killer cell leukemia =
aER; amenorrhea = aeS; alphavirus infections = aEs; biliary tract disease = aES; bulbospinal neuronopathy = aes; acute
kidney tubular necrosis = aET; loss of scalp hair = aet; anterior uveitis idiopathyic = aEt; 46 xy gonadal dysgenesis =
aeT; west nile fever = aEU; male breast cancer = aeU; primary disease = aeu; alphaviral infection = aEu; female infertility
associated with anovulation = aev; pediatric human immunodeficiency virus infection = aEv; mycobacterium avium complex
disease = aEV; benign breast phyllodes tumor = aeV; netherton syndrome = aEW; apocrine gland carcinoma = aew; benign
prostate phyllodes tumor = aeW; recurrent aphthous ulcer = aEw; idiopathyic hirsutism = aex; lymphoma, extranodal nk-t-cell =
aEx; myelomaonocytic leukemia = aEX; pleomorphic adenoma carcinoma = aeX; hereditary motor neuron disease = aey; mitochondrial
pathyology = aEY; hereditary allergy = aEy; tenosynovial giant cell tumor = aeY; neuropathy, hereditary sensory, with
spastic paraplegia, autosomal recessive = aEZ; chancroid = aEz; pseudohermaphrodite (non-specific) = aez; sertoli cell
tumor = aeZ; alzheimer's disease = af; fryns-aftimos syndrome = AF; cap myopathy = Af; stomach carcinoma = aF; placental
site trophyoblastic tumor = afa; hereditary sensory and autonomic neuropathyies = aFa; transfusion related acute lung
injury = aFA; retinoschisis 1, x-linked, juvenile = afA; pleomorphic adenoma = afb; alveolar bone loss = aFb; hair disease =
afB; immune neutropenia = aFB; relative polycythemia = aFC; hyperargininemia = afC; enterovirus infections = aFc; infertility =
afc; gram-negative bacterial infections = aFd; antibody deficiency due to defect in cd19 = aFD; hereditary breast ovarian
cancer = afd; carbambyl phosphate synthetase i deficiency disease = afD; hunger = afE; pulmonary sclerosisng hemangioma =
afe; pemphigus foliaceus = aFE; periodontal attachment loss = aFe; acne = aff; community-acquired infections = aFf;
embryonal testis carcinoma = afF; antibody deficiency syndrome = aFF; viral meningitis = aFG; adams-oliver syndrome =
afG; aicardi syndrome = afg; chlamydia pneumoniae infection = aFg; microphthalmia, syndromeic 7 = afH; prurigo = aFh;
atrophyic muscular disease = afh; alveolar pyorrhea = aFH; arthritis by pattern of joint involvement = aFI; coronary
spastic angina = afI; congenital heart block = afi; septicemia due to gram-negative organism, unspecified = aFi; condyloma =
aFJ; gingival fibromatosi = afj; cholestasis in newborn = aFj; nephrotic syndrome, idiopathyic, steroid-resistant =
afJ; acute lyme disease = aFk; neutropenia, severe congenital, autosomal recessive 3 = afK; genital infection = aFK;
arthrogryposis renal dysfunction cholestasis syndrome = afk; coronary arteritis = aFl; giving-way = afl; birbeck granule
deficiency = aFL; nephrotic syndrome, type 8 = afL; acute malaria = aFm; respiratory tract infections = afm; lichen simplex
chronicus = afM; cavitation of lung = aFM; mannose-binding protein deficiency = afn; immunodeficiency due to defect in
cd3-zeta = aFN; neurodermatitis = afN; distal colitis = aFn; obstructive disease = afo; sepsis of the newborn = aFO;
tomaculous neuropathy = afO; refractory cytopenia with multilineage dysplasia (rcmd) = aFo; slowed nerve conduction velocity,
autosomal dominant = afP; acute arthritis = aFp; copd, severe early-onset = afp; bacterial sepsis of newborn = aFP; peeling
skin syndrome = aFQ; basal cell carcinoma = afQ; bacterial diarrhoea = aFq; bronchiectasis = afq; keratoacanthoma = afr;
x-linked retinitis pigmentosa = afR; bacterial vaginosis = aFr; lymphoproliferative syndrome 2 = aFR; echinococcosis =
afS; sympathyetic ophthalmia = aFS; burning mouth syndrome = aFs; tonsillitis = afs; cardiac sarcoidosis = afT; appendicitis =
aFt; inflammatory neuropathy = aFT; myoclonic epilepsy, familial infantile = aft; paracoccidioidomycosis = aFU; choledochal
cyst = aFu; aarskog-scott syndrome = afU; vesicular stomatitis = afu; rheumatoid vasculitis = aFV; gitelman syndrome =
afV; atrioventricular bloc = afv; eczema herpeticum = aFv; pulmonary paracoccidioidomycosis = aFW; mean platelet volume =
afW; heterotopia, periventricular, autosomal recessive = afw; nephropathy with pretibial epidermaolysis bullosa and
deafness = aFw; fuchs' heterochromic uveitis = aFX; opioid abuse = aFx; mohr-tranebjaerg syndrome = afX; periventricular
heterotopia = afx; hypoplasia of corpus callosum = afy; mental retardation, x-linked 46 = afY; myasthenic syndrome, congenital,
slow-channel = aFY; capillary malformations, congenital, 1 = aFy; double cortex = afZ; focal segmental glomerulosclerosis
3, susceptibility to = aFZ; periventricular nodular heterotopia = afz; thalassemia minor = aFz; familial glucocorticoid
deficiency 1 = aG; nemaline myopathy, autosomal recessive = Ag; severe hypoxic ischemiac encephalyopathy = AG; thrombophilia =
ag; schizoaffective disorder, bipolar type = aga; lymphoid depletion = aGA; talkativeness = aGa; macular degeneration,
age-related, 8 = agA; retinal pigment epithelial detachment = agB; immunodeficiency 18 = aGB; frasier syndrome = aGb;
prodromal symptoms = agb; t lymphocyte deficiency = aGC; schizotypal personality disorder = agc; methylmalonic aciduria
due to transcobalamin receptor defect = aGc; choroiditis = agC; thrombocytopenia 1 = aGd; immunodeficiency 17 = aGD;
retinal drusen = agD; velocardiofacial syndrome = agd; okt4 epitope deficiency = aGE; hypopituitaris = agE; gaucher disease,
type i = aGe; acquired polycythemia = age; gaucher disease, type iii = aGf; kidney hypertrophy = agf; infection by human
herpesvirus 7 = aGF; ovary serous adenocarcinoma = agF; mucopolysaccharidosis ii = agG; immunodeficiency with hyper-igm,
type 3 = aGG; hyperekplexia and epilepsy = agg; heat stroke = aGg; quebec platelet disorder = aGh; mental retardation,
autosomal dominant 12 = agh; porokeratosis, disseminated superficial actinic 1 = agH; subacute combined degeneration =
aGH; retinal vasculitis = agI; cancer regression = aGi; osteoporosis (osteopenia) = aGI; scaphycephaly = agi; neurotic
disorder = agJ; synostosis = agj; rupture, spontaneous = aGJ; acute-on-chronic liver failure = aGj; coffin-siris syndrome =
agk; fibrous papule of nose = aGk; anxiety, separation = agK; sclerosis of the skin = aGK; immunoglobulin deposition
disease = aGL; fibrous papule of face = aGl; angelman syndrome = agl; metachromatic leukodystrophy, congenital type =
agL; metachromatic leukodystrophy, juvenile type = agM; maple syrup urine disease = agm; lymphangioma = aGm; spastic
dysphonia = aGM; adult disease = agn; metachromatic leukodystrophy, adult type = agN; poxviridae infections = aGN; fibrous
histiocytoma = aGn; meckel syndrome type 1 = ago; arylsulfatase a deficiency = agO; cutaneous fibrous histiocytoma =
aGo; meige syndrome = aGO; platelet glycoprotein iv deficiency = aGp; diverticulitis = aGP; joubert syndrome 8 = agp;
chronic psychosis = agP; erythema infectiosum = aGQ; retinitis pigmentosa with or without situs inversus = agq; pseudoarylsulfatase
a deficiency = agQ; plasmodium falciparum blood infection level = aGq; retinitis pigmentosa 55 = agr; sulfatiduria =
agR; coronary heart disease, susceptibility to, 7 = aGr; oral hairy leukoplakia = aGR; bardet-biedl syndrome 3 = ags;
pterygium of conjunctiva and cornea = aGs; cavity = agS; toxoplasmosis, cerebral = aGS; septal hypertrophy = aGt; immunodeficiency
congenital = aGT; spastic paraplegia 61, autosomal recessive = agt; unverricht-lundborg syndrome = agT; demyelinating
polyneuropathy = agU; ciliary dyskinesia, primary, 23 = agu; exercise-induced angina = aGu; autoimmune thrombocytopenia =
aGU; breast liposarcoma = aGv; hyperaldosteronis = agv; mucosulfatidosis = agV; hyper-igm immunodeficiency, x-linked,
with ectodermal dysplasia, hypohidrotic = aGV; lymphocytosis - absolute = aGw; cardiac symptom = agW; currarino triad =
agw; tuberculoid leprosy = aGW; lubs x-linked mental retardation syndrome = agX; mixed connective tissue disease =
aGX; severe combined immunodeficiency, autosomal recessive, t cell negative, b cell positive, nk cell positive = aGx;
incontinentia pigmenti, familial male-lethal type = agx; human immunodeficiency virus, type 2 [hiv-2] = agy; urban schosser
spohn syndrome = aGY; severe combined immunodeficiency, autosomal recessive, t cell-negative, b cell-positive, nk cell-negative =
aGy; 22q11 deletion syndrome = agY; bloch-sulzberger syndrome = agz; cognitive function 1, social = agZ; immunodeficiency
19 = aGz; incoherent thinking = aGZ; bjornstad syndrome = aH; dengue disease = AH; intranuclear rod myopathy = Ah; nephrotic
syndrome = ah; farber lipogranulomatosis = ahA; specific language impairment = aha; stage ib cervical cancer = aHa; myxoid
chondrosarcoma = aHA; pneumonia due to gram negative bacteria = aHb; glaucoma 1, open angle, f = ahB; congenital fibrosarcoma =
aHB; facial hemihypertrophy = ahb; intraductal papilloma = aHc; visual symptoms = ahC; synpolydactyly = aHC; autistic
behavior = ahc; chromosome 22q11.2 microduplication syndrome = ahd; gliomatosis cerebri = aHd; orofaciodigital syndrome =
ahD; aquagenic pruritus = aHD; obsession = ahe; hirschsprung disease 1 = ahE; omodysplasia = aHe; basophilic leukemia =
aHE; patent foramen ovale = ahf; wound, healed = aHF; cchs with hirschsprung disease = ahF; hemolytic uremic syndrome,
atypical, susceptibility to, 2 = aHf; mucoepidermaoid tumor = ahG; diarrhea-negative hemolytic uremic syndrome = aHg;
asperger syndrome = ahg; buffalo hump = aHG; lichen disease = aHH; streptococcus pyogenes infection = aHh; cerebrovascular
occlusion = ahH; chondrodysplasia punctata, brachytelephalangic = ahh; shipyard eye = aHi; hirschsprung's disease = ahI;
x-linked chondrodysplasia punctata 1 = ahi; agammaglobulinemia 3, autosomal recessive = aHI; islet cell tumor = ahJ;
chondrodysplasia punctata = ahj; autoimmune hemolytic anemia = aHj; agammaglobulinemia 6, autosomal recessive = aHJ;
olfactory neuroblastoma = ahk; thymus squamous cell carcinoma = aHk; vitreoretinochoroidopathy = ahk; chromosome 12,
12p trisomy = aHK; cd59 deficiency = aHl; differentiating neuroblastoma = ahl; lissencephaly, x-linked, 2 = ahL; neuroborreliosis =
aHL; nephrotic syndrome in children = aHM; cromer blood group system = aHm; taste sour = ahM; epileptic encephalyopathy,
early infantile, 1 = ahm; primary progressive multiple sclerosis = ahN; swine vesicular disease = aHn; functional proteinuria =
aHN; mental retardation, x-linked, with or without seizures, arx-related = ahn; hyperalgesia, primary = ahO; immunodeficiency,
common variable, 6 = aHO; proud syndrome = aho; intravascular hemolysis = aHo; partington x-linked mental retardation
syndrome = ahp; hemoglobinuria = aHp; anticoagulants causing adverse effects in therapeutic use = aHP; autonomic nervous
system imbalance = ahP; mastitis-metritis-agalactia syndrome = aHq; cryptogenic west syndrome = ahq; decubitus ulce =
ahQ; poisoning by anticoagulant = aHQ; bladder cancer stage = aHR; paroxysmal nonkinesigenic dyskinesia = ahR; brain
cyst = ahr; 22q13.3 deletion syndrome = aHr; pseudohypoaldosteronism = ahS; partington syndrome = ahs; lipomatosis, multiple =
aHs; dental pulp exposure = aHS; cd8 deficiency, familial = aHT; hexadactyly = aHt; hydranencephaly = aht; skin/hair/eye
pigmentation, variation in, 9 = ahT; pigment deposition = ahU; myoma = aHu; ideomotor apraxia = ahu; multiple sclerosis,
chronic progressive = aHU; neoplasms, glandular and epithelial = ahv; plasmodium ovale infection = aHV; porphyria cutanea
tarda = aHv; skin pigmentation disorder = ahV; urea cycle disorder = ahW; acute hiv infection = aHW; lipomatous cancer =
aHw; combined saposin deficiency = ahw; congenital mesoblastic nephroma = aHx; pineal gland cancer = ahX; jankovic
rivera syndrome = ahx; opitz trigonocephaly syndrome = aHX; glomus tumor = aHY; pseudohypoparathyroidism type 1b = ahy;
cellular myxoid liposarcoma = aHy; asparagine synthetase deficiency = ahY; microcephaly, congenital = ahZ; saposin d
(disease) = ahz; intravenous leiomyomatosis = aHz; blastema predominant kidney wilms' tumor = aHZ; arthritis = ai; peroxisome
biogenesis disorders = aI; nemaline myopathy, autosomal dominant = Ai; dysostosis = AI; hypertyrosinemia, richner-hanhart
type = aiA; deafness, autosomal recessive = aIA; secondary hemochromatosis = aIa; aminoacylase 2 deficiency = aia; cerebral
degeneration = aib; juvenile macular degeneration and hypotrichosis = aIB; unilateral multicystic dysplastic kidney =
aIb; ductal breast carcinoma = aiB; antithrombin iii deficiency = aiC; congenital hypotrichia = aIC; pectus excavatum =
aIc; canavan disease = aic; purine nucleoside phosphorylase deficiency = aiD; no-reflow phenomenon = aID; neonatal
anemia = aId; choledocholithiasis = aid; cone-rod dystrophy 15 = aIE; cerebellar ataxia, cayman type = aiE; pica disease =
aIe; ectopia lentis, spontaneous filtering blebs, and craniofacial dysmorphism = aie; vascular degeneration = aif;
keratoacanthoma familial = aIf; clear cell sarcoma, of tendons and aponeuroses = aif; leber congenital amaurosi = aIF;
pemphigus and fogo selvagem = aIg; round cell sarcoma = aiG; hermaphroditis = aIG; hepatobiliary benign neoplasm = aig;
myoepithelioma = aiH; corneal dystrophy and perceptive deafness = aIH; microcephaly, primary autosomal recessive, 5 =
aih; meier-gorlin syndrome 5 = aIh; cerebellar lesion = aiI; meier-gorlin syndrome = aIi; chandler syndrome = aII; autosomal
recessive primary microcephaly = aii; hereditary wilms' tumor = aIj; primary microcephaly = aij; varicose ulcer = aiJ;
vascular ischemia = aIJ; intervertebral disc disease = aik; acquired atrophy of ovary = aIK; polyp of corpus uter = aIk;
secondary infection = aik; vulvar disease = aIl; heartburn = aiL; congenital torticollis = ail; melanoma, cutaneous malignant,
susceptibility to, 3 = aIL; childhood kidney cell carcinoma = aim; lobular neoplasmia = aIm; inflammatory mfh = aIM;
west nile viral infection = aiM; spongiotic dermatitis = aIn; intracortical osteogenic sarcoma = aIN; thromboembolism
in children = ain; infective dermatitis = aiN; gastric signet ring cell adenocarcinoma = aIo; recurrent depression =
aiO; multiple pathyologies = aIO; masa syndrome = aio; hyperammonemia = aip; malignant mesenchymoma = aIp; chronic myelopathy =
aiP; developmental reading disorder = aIP; isodicentric chromosome 15 syndrome = aIQ; argininosuccinic acid synthetase
deficiency, complete = aiq; hyperplastic lymph node = aIq; inflammatory disease of the central nervous system = aiQ;
supraglottis squamous cell carcinoma = aIR; familial tremor = air; invasive tubular breast carcinoma = aIr; central nervous
system lesion = aiR; ectodermal dysplasia, ectrodactyly, and macular dystrophy = aIs; microcephaly, primary autosomal
recessive, 3 = aIS; bone marrow disease = aiS; citrullinemia = ais; transient myeloproliferative disorder of down syndrome =
aIT; bohring syndrome = ait; mental retardation, autosomal dominant 3 = aIt; rna virus infections = aiT; hypereosinophilia =
aiu; inflammatory bowel disease 10 = aiU; epileptic encephalyopathy, early infantile, 2 = aIU; hypotrichosis = aIu;
nance-horan syndrome = aIV; single gene defect = aiv; cryptococcosis = aiV; compulsive personality disorder = aIv; vacuolar
myopathy = aiW; anemia hemoglobin = aiw; gastric cancer stage = aIw; rett syndrome, atypical = aIW; neonatal hemochromatosis =
aIX; secondary malignant neoplasm = aix; aicar transformylase inosine monophosphate cyclohydrolase deficiency = aiX;
usher syndrome, type i = aIx; spastic paraplegia 3, autosomal dominant = aiY; bainbridge-ropers syndrome = aiy; myoclonic
encephalyopathy = aIY; usher syndrome, type id = aIy; plexiform neurofibroma = aiz; spastic paraplegia 10, autosomal
dominant = aiZ; usher syndrome, type 1d = aIz; blackout = aIZ; moyamoya disease 1 = aJ; actin-accumulation myopathy =
Aj; diabetes mellitus = aj; tonsil cancer = AJ; neuropathy, hereditary sensory, type id = aja; leukemiac infiltration =
ajA; familial primary gastric lymphoma = aJA; nodular ganglioneuroblastoma = aJa; hereditary autosomal dominant spastic
paraplegia = ajb; invasive bladder transitional cell carcinoma = aJb; autosomal aneuploidy = aJB; metastases to ovary =
ajB; chronic pelvic pain of female = ajC; cerebral palsy, spastic, diplegiac = ajc; superficial gastritis = aJC; immunodeficiency
13 = aJc; idiopathyic interstitial pneumonia = ajd; multiple endocrine neoplasmia, type iv = aJd; proliferative verrucous
oral leukoplakia = aJD; chondritis = ajD; megacolon, not hirschsprung's = aJe; polychondritis = ajE; retinomas (translucent,
grayish retinal mass protruding into the vitreous) = aJE; donohue syndrome = aje; neuropathy, hereditary sensory, type
if = ajf; tubular adenomas and adenocarcinomas = aJF; megacolon = aJf; pelvic pain = ajF; myoclonic cerebellar dyssynergia =
ajG; clear cell adenofibroma = aJg; vaginal cancer = aJG; at, complementation group e = ajg; cystadenofibroma = aJh;
ataxia-telangiectasisa variant = ajh; cervicitis = aJH; sly syndrome = ajH; peniale neoplasm = aJI; intrauterine growth
retardation, metaphyseal dysplasia, adrenal hypoplasia congenita, and genital anomalies = aJi; diurnal dystonia = aji;
dacryoadenitis = ajI; gestational choriocarcinoma = aJj; anus cancer = aJJ; contralateral breast cancer = ajj; central
nervous system leukemia = ajJ; relapsing polychondritis = ajK; supratentorial cancer = aJK; polyomavirus infections =
ajk; ulnar-mammary syndrome = aJk; sarcoma family syndrome of li and fraumeni = aJl; ovarian brenner tumor = aJL; krukenberg
carcinoma = ajL; aggressive periodontitis, generalized = ajl; trichoepithelioma multiple familial = aJm; aggressive non-hodgkin
lymphoma = ajm; liver angiosarcoma = aJM; brugada syndrome = ajM; radiation damage = ajn; cerebellar liponeurocytoma =
ajN; melanoma-pancreatic cancer syndrome = aJn; bladder squamous cell carcinoma = aJN; amelanotic melanoma = aJO; lymphoblastic
leukemia in children = ajo; melanoma, cutaneous malignant, susceptibility to, 2 = aJo; retinal nonattachment, nonsyndromeic
congenital = ajO; eye carcinoma in situ = ajP; cervical cancer stage = ajp; melanoma astrocytoma syndrome = aJp; papillary
squamous carcinoma = aJP; oculopalateocerebral syndrome = aJq; benign meningioma = aJQ; hodgkin's disease in children =
ajq; persistent hyperplastic primary vitreou = ajQ; cerebral small vessel diseases = aFr; isolated cervical dystonia =
ajr; vascular: intracranial = ajR; brain stem astrocytic neoplasm = aJR; microcystic adenoma = aJS; idiopathyic polypoidal
choroidal vasculopathy = ajs; physical function = aJs; neuropathy ataxia and retinis pigmentosa = ajS; keratinizing squamous
cell carcinoma = aJT; gastro-esophageal reflux disease with esophagitis = alt; renal tubular acidosis = ajT; eales disease =
ajt; cervix small cell carcinoma = aJU; laryngopharyngeal cancer = aju; leigh disease = ajU; chromosome 10, monosomy
10q = aJu; pineal region germinoma = aJV; gastrointestinal symptom = ajV; li-fraumeni syndrome = ajv; glandular neoplasms =
aJv; hypopharynx cancer = aJW; infantile malignant osteopetrosis = ajW; neuroblastoma recurrent = aJw; coats disease =
ajw; sensorineural hearing loss, bilateral = ajX; human t-cell lymphotrophyic virus, type i [htlv-i] = aJx; infantile
refsum disease = ajx; juvenile myelomonocytic leukemia = aJX; meningitis, meningococcal = ajy; vulval intraepithelial
neoplasmia grade 3 = aJy; alcohol dependence = aJY; deformity of bone = ajY; meningitis, pneumococcal = ajz; leukemia,
chronic lymphocytic, susceptibility to, 2 = aJz; holoprosencephaly 11 = aJZ; recurrent ulcer = ajZ; lewy body dementia =
ak; metachromatic leukodystrophy = AK; glucocorticoid deficiency = ak; neuromuscular disease = Ak; lipomatosis = aKA;
non-st elevation (nstemi) myocardial infarction = aka; muscle relaxation = akA; meier-gorlin syndrome 4 = aka; hyperostosis =
akb; wolman disease = aKB; barrett's esophague akb; akb; central core myopathy = akB; darier disease, segmental = akC;
bile reflux = akc; eosinophilic esophagitis = akc; disseminated eosinophilic collagen disease = akC; loeffler syndrome =
aKD; dyspepsia = akd; glandular cystitis = aKd; darier disease, acral hemorrhagic type = akD; keratosis follicularis =
akE; speech and language disorder = ake; mucinous intrahepatic cholangiocarcinoma = ake; pancreatic insufficiency =
aKE; benign epilepsy with centrotemporal spikes = akf; pancreatic malabsorption = aKF; myocardial stunning = akF; deafness,
autosomal dominant 4 = aKf; exocrine pancreatic insufficiency = aKG; episodic ataxia with nystagmus = akg; hailey-hailey
disease = akG; deafness, autosomal dominant 4b = aKg; follicular carcinoma, widely invasive = aKh; hemiplegiac migraine,
familial type 2 = akh; sphingolipidosis = akH; congenital hypoplasia of thymus = aKH; deafness, autosomal recessive 12 =
akI; alternating hemiplegia of childhood = aki; chromosome 10, monosomy 10p = aKI; colon carcinoma in situ = aKi; alternating
hemiplegia of childhood 1 = akj; microcephaly, primary autosomal recessive, 6 = aKJ; encephalyomyelitis = akJ; myeloid
tumor suppressor = aKj; migraine, familial basilar = akk; familial acute myeloid leukemia with mutated cebpa = aKk; pericentrin =
aKK; spinocerebellar ataxia, x-linked 1 = akK; microcephaly 8, primary, autosomal recessive = aKL; ventilator-induced
lung injury = akl; histiocytic leukemia = aKl; olivopontocerebellar atrophy = akL; mitochondrial complex v (atp synthase)
deficiency, nuclear type 4 = akM; microcephaly 9, primary, autosomal recessive = aKM; specific granule deficiency = aKm;
hemiplegiac migraine = akm; seckel syndrome 5 = aKN; polyarteritis nodos = aKn; spinocerebellar ataxia 17 = akN; epilepsy
and migraine = akn; mitochondrial complex v (atp synthase) deficiency, nuclear type 3 = akO; transient neurological symptoms =
ako; symptoms/complaints of eyelids = aKo; ataxia telangiectasis = aKO; amyloidosis, hereditary, transthyretin-related =
aKp; migraine with typical aura = akp; nephronophthisis 15 = aKP; complex v deficiency = akP; benign familial neonatal
epilepsy = akq; maturity-onset diabetes of the young, type 8, with exocrine dysfunction = aKq; morbid obesity and spermatogenic
failure = aKQ; kearns-sayre syndrome = akQ; joubert syndrome 5 = aKR; marie unna congenital hypotrichosis = akR; cerebral
palsy, ataxiac, autosomal recessive = aKr; episodic ataxia = akr; hereditary sensory autonomic neuropathy, type 2 = akS;
senile cardiac amyloidosis = aKs; senior-loken syndrome 6 = aKS; dystonia 12 = aks; leber congenital amaurosis 10 = aKT;
dystonia 3, torsion, x-linked = akt; benign symmetrical lipomatosis = aKt; acute hepatitis e = akT; meckel syndrome,
type 4 = aKU; cervix disease = akl; alternating hemiplegia of childhood 2 = aku; late-onset disorders = aKu; capos syndrome =
akv; anogenital venereal wart = akV; protein misfolding disorders = aKv; bardet-biedl syndrome 14 = aKV; joubert syndrome
15 = aKW; greig cephalyopolysyndactyly syndrome = akW; ulnar nerve entrapment = aKw; neonatal infection = akw; atrophy
of pancreas = aKx; mosaic variegated aneuploidy syndrome 2 = aKX; motor disorder = akx; cowpox = akX; seckel syndrome
6 = aKY; acute respiratory disease = aKy; esophagitis = akY; chronic mountain sickness = aky; brody myopathy = akz; mental
retardation, x-linked, with epilepsy = akZ; alcohol-induced chronic pancreatitis = aKz; nephronophthisis 2 = aKZ; ophthalmoplegia =
Al; hellp syndrome = AL; zellweger spectrum = aL; liver cirrhosis = al; cutaneous telangiectasisa and cancer syndrome,
familial = alA; hemorrhagic colitis = aLA; retinitis pigmentosa 26 = aLa; parkinsonism with spasticity, x-linked = ala;
renal disease (acute) nos = aLB; blepharophimosis = alB; dna virus infections = alb; ichthyosis, congenital, autosomal
recessive 9 = aLb; wrinkly skin syndrome = alc; nonexudative age-related macular degeneration = aLC; williams-beuren
syndrome = alC; primary hypercholesterolemia = aLc; cutis laxa, autosomal recessive, type iia = ald; heterotaxy, visceral,
6, autosomal = aLd; costeff optic atrophy syndrome = alD; hiv nephropathy = aLD; enlarged vestibular aqueduct = ale;
immunodeficiency syndrome, variable = alE; ocular sarcoidosis = aLE; van der woude syndrome 2 = aLe; renal tubular acidosis,
distal, autosomal recessive = alf; alpha-thalassemia myelodysplasia syndrome = alF; hemolytic uremic syndrome, atypical,
susceptibility to, 4 = aLf; verotoxigenic escherichia coli gastrointestinal tract infection = aLF; psychomotor impairment =
alG; renal tubular acidosis, distal, autosomal recessive, with late-onset sensorineural hearing loss = alg; complement
factor b deficiency = aLg; complement abnormality = aLG; endocapillary glomerulonephritis = aLH; necrotizing ulcerative
gingivitis = aLh; renal tubular acidosis, distal, with progressive nerve deafness = alh; generalized pruritus = alH;
panuveitis = aLI; rubinstein-taybi syndrome = alI; conotruncal cardiac defects = aLi; neural hearing loss = ali; relapsing
fever = aLJ; adult astrocytic tumorur = alJ; distal myopathy, nonaka type = alj; heterotaxy, visceral, 2, autosomal =
aLj; occipital horn syndrome = alk; transposition of the great arteries, dextro-looped 2 = aLk; tick-borne relapsing
fever = aLK; coffin-lowry syndrome = alK; misalignment = aLl; spinocerebellar ataxia 2 = alL; spinal muscular atrophy,
distal, x-linked 3 = all; congenital afibrinogenemia = aLL; proliferative glomerulonephritis = aLM; polysplenia syndrome =
aLm; salla disease = alm; spinocerebellar degenerations = alM; cfhr5 deficiency = aLN; hypocupremia = aln; progressive
cerebellar degeneration = alN; double outlet right ventricle = aLn; thin basement membrane disease = aLO; blotchy = alo;
decreased tendon reflex = alO; transposition of the great arteries, dextro-looped 1 = aLo; disorder of complement = aLP;
complement factor d deficiency = aLp; spinocerebellar ataxia 10 = alP; sialuria = alp; neisseriaceae infections = aLq;
hemolytic uremic syndrome, atypical, susceptibility to, 3 = aLQ; spinocerebellar ataxia 36 = alQ; deficiency of ferroxidase =
alq; kayser-fleischer ring = alr; macular degeneration, age-related, 13 = aLR; tdp-43 proteinopathyies = alR; arthus
reaction = aLr; cirrhosis, familial, with pulmonary hypertension = als; rigid spine syndrome = aLs; acute hemorrhagic
leukoencephalyitis = aLS; autosomal dominant parkinsonism = alS; heart cancer = alt; action tremor = alT; macular degeneration,
age-related, 4 = aLt; nemaline myopathy 7 = aLT; calcaneal apophysitis = alU; cerebellar ataxia, mental retardation,
and dysequilibrium syndrome 4 = alu; properdin deficiency, x-linked = aLU; complement factor i deficiency = aLu; basal
laminar drusen = alv; end-position nystagmus = alV; attention deficit and disruptive behavior disorders = alv; properdin
deficiency, type iii = aLV; properdin deficiency, type ii = aLW; reducing-body myopathy = aLw; nervous system--degeneration =
alW; cholestasis, intrahepatic, of pregnancy, 1 = alw; simple febrile seizure = aLX; scapuloperoneal myopathy = aLx;
dyssomnias = alX; idiopathyic chronic pancreatitis = alx; mitochondrial complex v (atp synthase) deficiency, nuclear
type 1 = aly; myotonia congenita = aLY; cataplexy and narcolepsy = alY; properdin deficiency disease = aLy; mitochondrial
complex v (atp synthase) deficiency, atpaf2 type = alz; bronchiectasis with or without elevated sweat chloride 1 = aLZ;
multifocal choroiditis = aLz; amyloid polyneuropathy type i = alZ; nemaline myopathy = Am; endometrial adenocarcinoma =
AM; alpha I-antitrypsin deficiency = am; nicotinamide adenine dinucleotide coenzyme q reductase deficiency = aM; pure
autonomic failure = ama; genetic diseases, x-linked = amA; colonic disease = aMA; lactose intolerance, adult type = aMa;
osteomalacia = amB; lactase persistence = aMb; effects of heat = amb; typhoid fever = aMB; hepatobiliary disease = aMC;
x-linked hypophosphatemiac rickets = amC; hereditary antithrombin iii deficiency = amc; pancreatitis idiopathyic = aMc;
ileus = aMD; exotropia = amd; conotruncal anomaly face syndrome = amD; pancreas divisum = aMd; echogenic bowel = aMe;
mite infestation = aME; protein c deficiency = ame; caudal duplication anomaly = amE; burkholderia infections = aMf;
bicuspid aortic valve = amF; choroidal dystrophy, central areolar 2 = amf; congenital secretory chloride diarrhea = aMF;
uterus leiomyosarcoma = amg; 3b-hydroxysteroid dehydrogenase deficiency = aMG; oligodontia-colorectal cancer syndrome =
amG; child nutrition disorders = aMg; epidermaolysa bullosa simplex and limb girdle muscular dystrophy = aMH; male
infertility with large-headed, multiflagellar, polyploid spermatozoa = amh; congenital absence of vas deferens = aMh;
tooth agenisi, selective, 1 = amH; congenital absence of kidney = aMi; primary infertility = ami; thyroid cancer, follicular =
aMI; hypodontia = amI; dysmorphic features = amj; autoimmune pancreatitis = aMj; cleft palate with cleft lip = amJ;
severe hereditary factor viii deficiency disease = aMJ; wasting syndrome = amK; parainfluenza = aMk; nephrogenic syndrome
of inappropriate antidiuresis = amk; hormone related neoplasm/cancer = aMK; hypertelorism with esophageal abnormality
and hypospadias = aml; antibiotic-associated diarrhea = amL; fibrocystic disease of pancreas = aMl; temper tantrum =
aML; pulmonary mycobacterium avium complex infection = aMm; driving under influence of alcohol or drugs = amM; familial
diabetes insipidus = amm; congenital myasthenic syndrome with episodic apnea = aMM; cytomaegalovirus encephalyitis =
amN; epileptic encephalyopathy, childhood-onset = aMN; chronic lung injury = aMn; familial central diabetes insipidus =
amn; infectious disorder of bronchus = aMo; scoliosis, idiopathyic, susceptibility to, 3 = aMO; multiple trauma = amo;
amyloidosis, familial visceral = amO; idiopathyic bronchiectasis = aMp; paraproteinemias = amP; idiopathyic hypogonadotropic
hypogonadism = aMP; mixed sensory-motor polyneuropathy = amp; hemodialysis-associated amyloidosis = amQ; bilateral agenisi =
aMq; polydipsia, psychogenic = amq; hypogonadotropic hypogonadism 5 with or without anosmia = aMQ; distal ileal obstruction
syndrome = aMr; kallmann syndrome 5 = aMR; hypersexuality state = amr; peripheral neuroepithelioma = amR; plasmacytic
leukemia = amS; chronic emphysema = aMs; hemifacial microsomia = aMS; chronic hyponatremia = ams; dysthymic disorder =
amt; respiratory morbidity = aMt; blood group antigen p = amT; ear malformation = aMT; goldenhar syndrome = aMU; premature
ejaculation = amu; muscular dystrophy-dystroglycanopathy (congenital with brain and eye anomalies), type a, 11 = amU;
teratospermia = aMu; krause-kivlin syndrome = amV; facial paralysis = aMV; inappropriate adh syndrome = amv; abnormal
spermatogenesis = aMv; leukemia, large granular lymphocytic = amW; flat ductal epithelial atypia = amw; choanal atresia =
aMW; hypotonic dehydration = aMw; autism, susceptibility to, 18 = aMX; t-cell lymphocytosis = amX; aspartylglucosaminuria =
amx; paranasal sinus disease = aMx; melancholia = amy; gastrointestinal system disease = aMy; hodgkin's lymphoma, nodular
sclerosis = amY; right ventricular outflow tract obstruction = aMY; li-fraumeni syndrome 2 = aMZ; maxillary sinusitis =
aMz; azotemia = amz; multiple joint dislocations, short stature, craniofacial dysmorphism, and congenital heart defects =
amZ; van der woude syndrome = An; thyroid adenoma = AN; parkinson's disease = an; spastic paraplegia type 7 = aN; encounter
due to estrogen receptor status = aNa; extraskeletal myxoid chondrosarcoma = aNA; axenfeld-rieger syndrome = anA; spastic
paraplegia 26, autosomal recessive = ana; endometrial diseases = aNb; mobius syndrome = anB; chronic non-a non-b hepatitis =
aNB; congenital disorder of glycosylation type 2d = anb; childhood teratoma = aNc; congenital disorder of glycosylation,
type iiid = anc; fibrous bands = anC; fumarase deficiency = aNC; endometrioid ovary carcinoma = anD; ehlers-danlos syndrome
progeroid typ = and; skeletal malformation = aNd; social anhedonia = aND; muscular dystrophy-dystroglycanopathy (congenital
with brain and eye anomalies), type a, 13 = ane; congenital anomaly of skeletal bone = aNe; luft disease = aNE; diabetes
mellitus, insulin-dependent, 4 = anE; bardet-biedl syndrome 1 = anF; islet cell tumor syndrome = aNf; carnitine deficiency =
aNF; meckel syndrome, type 9 = anf; cecum adenocarcinoma = aNg; bardet-biedl syndrome 10 = anG; meckel syndrome, type
10 = ang; mitochondrial myopathy = aNG; pyruvate decarboxylase deficiency = aNH; bardet-biedl syndrome 12 = anH; hypercholanemia,
familial = anh; small intestine neuroendocrine neoplasm = aNh; brain stem glioma = aNI; zollinger-ellison syndrome =
aNi; mckusick kaufman syndrome = anI; hemolytic uremic syndrome, atypical, susceptibility to, 1 = ani; down syndrome
critical region = anj; collagenous colitis = aNj; bardet-biedl syndrome 2 = anJ; carnitine palmitoyltransferase ii deficiency =
aNJ; gastrinom = anK; lymphocytic colitis = aNk; head--cancer = ank; mandibular hypoplasia = aNK; asthma-related traits,
susceptibility to, 7 = aNl; cornealia de lange syndrome = aNL; bardet-biedl syndrome 4 = anL; periampullary adenocarcinoma =
anl; bardet-biedl syndrome 7 = anM; myopathy, myofibrillar, bag3-related = anm; herniated disk = aNm; megalocornea =
aNM; gram-positive bacterial infections = aNn; catatonia = aNN; cardiomyopathy, dilated, 1hh = ann; exencephaly = anN;
bardet-biedl syndrome 9 = anO; infectious pustular vulvovaginitis = ano; chitotriosisdase deficiency = aNo; chromosome
15q13.3 microdeletion syndrome = aNO; crescentic glomerulonephritis = anP; filariasis = aNp; airway constriction = aNP;
benzodiazepine abuse = anp; pemphigu = aNQ; nestor-guillermo progeria syndrome = anq; rapidly progressive glomerulonephritis =
anQ; myoglobinuria, recurrent = aNq; endometrial clear cell adenocarcinoma = anr; familial dilated cardiomyopathy =
anR; muscular dystrophy, congenital, megaconial type = aNr; bladder disease = aNR; myasthenic syndrome, congenital, fast-channel =
aNS; contiguous abcd1/dxs1375e deletion syndrome = anS; pontocerebellar hypoplasia, type 8 = aNs; multifocal motor
neuropathy = ans; myasthenic syndrome, congenital, postsynaptic slow-channel = aNT; cornea cancer = ant; htlv-ii infections =
anT; frontotemporal dementia, chromosome 3-linked = aNt; tumor predisposition syndrome = anu; tobacco dependencye =
aNU; developmental verbal dyspraxia = anU; amyotrophyic lateral sclerosis, chmp2b-related = aNu; estrogen resistance =
anV; benign pleural mesothelioma = anv; epilepsy, nocturnal frontal lobe, type 4 = aNV; anemia, hypochromic microcytic =
aNv; oral pharyngeal disorder = anW; cataract, posterior polar, 3 = aNw; benign intermediate mesothelioma = anw; autosomal
dominant nocturnal frontal lobe epilepsy = aNW; frontal lobe epilepsy = aNX; yersinia infections = anX; paraganglioma =
anx; duane retraction syndrome 2 = aNx; tuberculosis, extrapulmonary = anY; duane syndrome = aNy; iridogoniodysgenesis,
dominant type = any; lung cancer susceptibility 2 (disorder) = aNY; rieger eye malformation sequence = anz; smokers lung =
aNZ; manifest vertical squint = aNz; breast cancer (non-specific) premenopausal = anZ; aortic aneurysm, familial thoracic
6 = Ao; breast ductal carcinoma = AO; atherosclerotic occlusive disease = aO; cystic fibrosis = ao; bamforth syndrome =
aoa; uterine cervical dysplasia = aoA; epilepsy, nocturnal frontal lobe, type 1 = aOa; deafness, autosomal recessive
48 = aOA; electroencephalyogram, low-voltage = aOb; butyrylcholinesterase deficiency = aob; thomsen disease = aOB; venous
retinal branch occlusion = aoB; lipodystrophy, familial partial, type 5 = aOC; ring chromosome 20 syndrome = aOc; chronic
idiopathyic neutropenia = aoC; gestational trophyoblastic disease = aoc; bare lymphocyte syndrome, type ii, complementation
group a = aOD; superinfection = aoD; nocturnal epilepsy = aOd; apnea, postanesthetic = aod; north american indian childhood
cirrhosis = aOE; butyrylcholinesterase deficiency, fluoride-resistant, japanese type = aoe; schizophrenia 13 = aOe; oropharynx
(excludes nasopharynx) = aoE; weakness of limb = aoF; ichthyosis, leukocyte vacuoles, alopecia, and sclerosisng cholangitis =
aOF; myasthenic syndrome, congenital, type id = aOf; myelokathexis = aof; attention deficit-hyperactivity = aog; wolfram
syndrome 2 = aOG; canarypox = aoG; epilepsy, nocturnal frontal lobe, type 3 = aOg; mixed cryoglobulinemia = aoH; skin
manifestations = aOh; wolfram syndrome = aOH; adult-onset obesity = aoh; pena shokeir syndrome, type 1 = aOi; primary
cutaneous b-cell lymphoma = aoI; prolonged neuromuscular block = aoi; diabetes insipidu = aOI; growth hormone treatment =
aoJ; clinically isolated syndrome = aOJ; pregnancy complications, parasitic = aOj; churg-strauss syndrome = aoj; maple
syrup urine disease, type ia = aok; lymphocytic lymphoma = aoK; intrauterine infection = aOK; ehlers-danlos syndrome,
musculocontractural type 1 = aOk; follicular lymphoma, grade 1 = aoL; filarial elephantiasis = aOL; branched-chain ketoacid
dehydrogenase kinase deficiency = aol; ehlers-danlos syndrome 6b = aOl; kidney clear cell sarcoma = aOM; joint instability =
aOm; multicystic dysplastic kidney = aoM; neoplasms, plasma cell = aom; follicular neoplasm = aon; ventricular septal
defect 2 = aON; spondyloepiphyseal dysplasia, omani type = aOn; malignant lymphoma - lymphocytic, intermediate differentiation =
aoN; atrial septal defect 8 = aOO; in situ cancer = aoO; gastritis hpylori = aoo; apparent mineralocorticoid excess =
aOo; larsen syndrome, recessive type = aOp; plasma cell tumor, malignant = aop; primary cutaneous diffuse large cell
b-cell lymphoma = aoP; cervical dystonia, primary = aOP; adult-onset dystonias = aOQ; sertoli-leydig cell tumor of intermediate
differentiation = aOq; follicular non-hodgkin's lymphoma, large cell (clinical) = aoQ; overnutrition = aoq; hay-wells
syndrome = aOR; plasma cell neoplasm = aor; sertoli-leydig cell tumor = aOr; stage 1 endometrial cancer = aoR; colon
mucinous adenocarcinoma = aOs; rapp-hodgkin syndrome = aOS; enterochromaffin-like cell carcinoid = aoS; fetal hemoglobin
quantitative trait locus 5 = aos; sindbis virus infection = aoT; hemoglobinopathy = aot; macular corneal dystrophy, type
ii = aOt; propping zerres syndrome = aOT; codependency = aoU; corneal dystrophy, fuchs' endothelial, 1 = aou; corneal
dystrophy = aOu; ectrodactyly-cleft lip/palate syndrome = aOU; foot deformities, congenital = aOV; macular corneal dystrophy =
aOv; diamond-blackfan anemia 1 = aov; essential mixed cryoglobulinemia = aoV; minor oral aphthous ulceration = aOW;
temtamy preaxial brachydactyly syndrome = aOw; syndactyly, type iv = aow; endometrial cancer stage = aoW; extranodal
lymphoma = aoX; persistent polyclonal b-cell lymphocytosis = aox; cleft lip, isolated = aOX; tumoral calcinosis, hyperphosphatemiac,
familial = aOx; hyperostosis-hyperphosphatemia syndrome = aOy; follicular lymphoma, grade 3 = aoY; hypertrichosis congenital
generalized x-linked = aoy; limbal stem cell deficiency = aOY; depletion of mitochondrial dna = aoz; usher syndrome,
type 1j = aOz; clouston syndrome = aOZ; laryngeal papillomatosis = aoZ; obstructive lung disease = ap; mural thrombus
of heart = aP; aortic aneurysm, familial thoracic 1 = Ap; von economo's disease = AP; cerebral lymphoma = apA; acalculous
cholecystitis = aPA; atypical burkitt's lymphoma = apa; gray zone lymphoma = aPa; fibrillary astrocytoma = apB; gelatinous
drop-like corneal dystrophy = aPB; aphthous stomatitis = aPb; epithelial inclusion cyst = apb; protoplasmic astrocytoma =
apC; ulcerative stomatitis = aPc; cerebellar decompression injury = apc; deafness, autosomal recessive 29 = aPC; lymphoma,
follicular, grade 2 = apd; precursor b lymphoblastic lymphoma/leukemia = apD; exertional rhabdomyolysis = aPd; manz syndrome =
aPD; intraocular lymphoma = apE; lymphoma of intestine = aPE; punctate inner choroidopathy = aPe; venous hypertension =
ape; urinary outflow obstruction = apf; menstrual spotting = aPF; liver lymphoma = apF; hepatic encephalyopathy = aPf;
cold-induced sweating syndrome 2 = aPg; piebaldism = aPG; mast-cell leukemia = apG; disuse osteoporosis = apg; scheuermann's
disease = aPH; grade 3b follicular lymphoma = aph; xeroderma pigmentosaum = apH; cold-induced sweating syndrome 1 = aPh;
survival motor neuron spinal muscular atrophy = apI; congenital myotonia, autosomal recessive form = aPi; drugged state =
api; familial chronic mucocutaneous candidiasis = aPI; dermatitis and eczema = apJ; vulvovaginal candidiasis = aPJ;
morphea = apj; obscure african cardiomyopathy = aPj; precursor b-cell lymphoblastic lymphoma = apk; thymus neoplasms =
apK; acute gastric mucosal erosison = aPK; myotonia levior = aPk; intestinal pseudo-obstructio = aPL; myotonic disease =
aPl; nervous system cancer = apl; functionless pituitary adenoma = apL; cleft lip with or without cleft palate = apM;
thyroid lymphoma = apm; myotonic dystrophy type = aPm; ceroid lipofuscinosis, neuronal, 2 = aPM; juvenile disease = aPN;
rabies = apn; megalencephalyic leukoencephalyopathy with subcortical cysts = aPn; otofaciocervical syndrome 1 = apN;
htlv infections = apO; retinitis = apo; infantile disease = aPO; leukoencephalyopathy with ataxia = aPo; epilepsy with
grand mal seizures on awakening = aPp; oculopharyngeal muscular dystrophy = app; alkalemia = aPP; hodgkin lymphoma, nodular
lymphocyte predominance = apP; primary cutaneous large b-cell lymphoma of the leg = apQ; ceroid lipofuscinosis, neuronal
5 = aPQ; werdnig-hoffmann disease = apq; low molecular weight proteinuria with hypercalciuria and nephrocalcinosis =
aPq; parkinson disease 9 = aPR; idiopathyic cd4-positive t-lymphocytopenia = apR; granular cell tumor = apr; nephrolithiasis,
x-linked recessive, with renal failure = aPr; muscle benign neoplasm = aps; renal tubular transport disease = aPs; ceroid
lipofuscinosis, neuronal 8 = aPS; composite lymphoma = apS; dent disease = aPt; cryoglobulinemia = apt; high grade b-cell
non-hodgkin's lymphoma = apT; adult neuronal ceroid lipofuscinosis = aPT; ceroid lipofuscinosis, neuronal, 6 = aPU; osteopetrosis,
autosomal recessive 4 = aPu; hypercarotenemia and vitamin a deficiency, autosomal dominant = apU; female reproductive
endometrioid cancer = apu; ceroid lipofuscinosis, neuronal, 8 = aPV; osteopetrosis, autosomal dominant 2 = aPv; microphthalmia,
syndromeic 2 = apV; thoracic outlet syndrome = apv; thinness = aPW; bartter syndrome, type 4a = aPw; inverted papilloma =
apw; microphthalmia, syndromeic 1 = apW; cervical adenocarcinoma = apx; bartter syndrome, type 4b = aPx; anorexia and
bulimia syndrome = aPX; myeloid metaplasia = apX; eosinophilic leukemia = apY; mesenchymal chondrosarcoma = apy; bartter
syndrome, type 3, with hypocalciuria = aPy; winter depression = aPY; sleep-wake schedule disorder, advanced phase type =
aPZ; hypokalemic alkalosis = aPz; focal epithelial hyperplasia = apz; autosomal recessive agammaglobulinemia = apZ;
lung oat cell carcinoma = AQ; mural thrombus = aQ; multisystemic smooth muscle dysfunction syndrome = Aq; liver cancer =
aq; reversible cerebral vasoconstriction syndrome = aqA; circadian rhythm disorder = aQa; stereotypic movement disorder =
aqa; amaurosis hypertrichosis = aQA; delayed sleep phase = aQb; post-traumatic vegetative state = aqB; jalili syndrome =
aQB; mitochondrial complex iii deficiency = aqb; high-functioning autism = aQc; psychosocial stressor = aqC; mitochondrial
complex iii deficiency, nuclear type 1 = aqc; aortic valve insufficienc = aQC; adult attention deficit hyperactivity
disorder = aqD; q fever = aQd; leigh syndrome due to mitochondrial complex iii deficiency = aqd; iv drug use = aQD; gonadal
dysgenesis xx type deafness = aQe; ubiquinone dehydrogenase = aqe; spine degeneration = aqE; polysubstance abuse = aQE;
hepatic veins thrombosis = aqf; perrault syndrome 3 = aQf; nerve pain = aqF; perfectionism = aQF; hydroxyacyl-coa dehydrogenase,
type 2, deficiency = aqg; intrapartum fetal hypoxia = aqG; deafness, autosomal recessive 81 = aQg; abdominal discomfort =
aQG; dissociative disorder = aqH; tobacco addiction, susceptibility to = aqh; retinitis pigmentosa 61 = aQh; cold-induced
sweating syndrome = aQH; eating disorder = aqI; myopathy, congenital, compton-north = aQI; wounds, penetrating = aqi;
circling behavior = aQi; epilepsy, familial adult myoclonic, 5 = aQJ; pain, referred = aqj; riley-day syndrome = aqJ;
auditory system disease = aQj; pitt-hopkins syndrome = aQK; aggressive periodontitis, localized = aQk; heroin abuse =
aqk; wagr syndrome = aqK; cortical dysplasia-focal epilepsy syndrome = aQL; conversion disorder = aqL; social stress =
aql; adult atopic dermatitis = aQl; wernicke encephalyopathy = aqM; aortic stenosis symptomatic = aQm; language development
disorders = aQM; methamphetamine dependencye = aqm; postpartum depression = aqN; autism, susceptibility to, 15 = aQN;
telangiectasisa, hereditary benign = aQn; brain morphology = aqn; impulsive aggression = aqo; pitt-hopkins-like syndrome
1 = aQO; spinocerebellar ataxia 31 = aqO; porokeratosis = aQo; selective mutism = aQP; neurologic manifestations = aQp;
depressive episode, unspecified = aqp; marie's cerebellar ataxia = aqP; chronic depression = aqq; retinitis pigmentosa
49 = aQq; stuttering = aQQ; spinocerebellar ataxia 4 = aqQ; neurodegeneration with brain iron accumulation 6 = aQR; achromatopsia
3 = aQr; depressive disorder, treatment-resistant = aqr; hemangiom = aqR; achromatopsia 2 = aQs; pseudohypoparathyroidism
and pseudopseudohypoparathyroidism = aQS; bestrophyinopathy = aqS; psychiatric hospitalization = aqs; psychiatric diagnosis =
aqt; achromatopsia 1 = aQt; deafness, autosomal dominant 9 = aQT; doyne honeycomb retinal dystrophy = aqT; achromatopsia =
aQu; self-induced vomiting = aqu; dilated cardiomyopathy 3b = aqU; usher syndrome, type 1f = aQU; feeling despair =
aqv; late-onset retinal degeneration = aqV; retinitis pigmentosa 45 = aQv; dominant sensorineural hearing loss = aQV;
retinitis pigmentosa, concentric = aqW; progressive retinal atrophy = aQw; autoimmune sensorineural hearing loss = aQW;
emotional disturbance = aqw; clinical anxiety = aqx; stargardt disease 1 = aQx; macular dystrophy, vitelliform, adult-onset =
aqX; inner ear disease = aQX; acute disseminated encephalyitis = aqy; congenital hypothyroidis = aQy; ocular hypotension =
aQY; macular dystrophy, vitelliform = aqY; hypomagnesemia 6, renal = aQz; sarcoglycanopathyies = aqZ; vegetative state =
aqz; labyrinthine disease = aQZ; pancreatitis = ar; moyamoya disease 5 = Ar; gastrinoma = AR; nervous system disease =
aR; split-hand/foot malformation = arA; vestibular disease = aRa; fibrochondrogenesis 2 = aRA; macular dystrophy, concentric
annular = ara; grip strength decreased = aRB; limb defects = arB; congenital disorder of glycosylation, type 2g = aRb;
microcornea, rod-cone dystrophy, cataract, and posterior staphyloma = arb; exostosis = aRC; cerebrocostomandibular-like
syndrome = aRc; glucose-galactose malabsorption = arC; dmd-associated dilated cardiomyopathy = arc; cerebrocostomandibular
syndrome = aRd; sorsby's fundus dystrophy = ard; paroxysmal ventricular fibrillation = arD; smith-mccort dysplasia =
aRD; deformity of limb = are; erythrokeratoderma = arE; epidermaolysis bullosa, junctional, non-herlitz type = aRE; congenital
disorder of glycosylation, type iij = aRe; epidermaolysis bullosa, junctional, localisata variant (disorder) = aRF; congenital
disorder of glycosylation, type iii = aRf; myopathy with abnormal lipid metabolism = arf; carbohydrate intolerance =
arF; inherited platelet disorder = arG; congenital disorder of glycosylation, type iii = aRg; age-related osteoporosis =
arg; linear iga bullous dermatosis = aRG; high weight = arh; duane retraction syndrome = arH; shaheen syndrome = aRh;
junctional epidermaolysis bullosa mitis = aRH; lichen planus pemphigoides = aRI; idiopathyic juvenile osteoporosis =
ari; glanzmann's thrombasthenia = arI; congenital disorder of glycosylation type Ze = aRi; autoimmune skin disease =
aRJ; wet macular degeneration = arj; congenital disorder of glycosylation, type iih = aRj; leukocyte adhesion deficiency =
arJ; cataract, cortical, juvenile-onset = ark; localized junctional epidermaolysis bullosa = aRK; opitz gbbb syndrome,
x-linked = aRk; poikiloderma of kindler = arK; cicatricial pemphigoid = aRL; epidermaolysis bullosa letalis = arL; metaphyseal
chondrodysplasia schmid type = aRl; cataract, age-related nuclear = arl; mast-cell sarcoma = aRM; generalized epidermaolysis
bullosa simplex = arM; arthropathy, progressive pseudorheumatoid, of childhood = aRm; cataract, autosomal dominant =
arm; congenital anosmia = aRN; coxa vara (acquired) = aRn; cataract 12, multiple types = arn; rundown = arN; cartilage
disease = aRo; d-2-hydroxyglutaric aciduria 2 = aRO; cataract, autosomal dominant, multiple types 1 = aro; epidermaolysis
bullosa simplex = arO; short-sleeper = arP; vitamin d-resistant rickets, hereditary = arp; osteogenesis imperfecta, recessive
perinatal lethal = aRP; weaver syndrome = aRp; oi/eds combined syndrome = aRQ; choline deficiency disease = arQ; hyaloideoretinal
degeneration of wagner = aRq; dental fluorosis = arq; fibrochondrogenesis 1 = aRr; heterotaxy, visceral, 5, autosomal =
arR; vitamin d-resistant rickets = arr; spinal fractures = aRR; oral manifestations = aRS; lumbar disc herniation,
susceptibility to = aRs; rickets = ars; polycystic kidney disease 1 = arS; soft tissue injuries = aRT; saethre-chotzen
syndrome = art; vitreous syneresis = aRt; renal dysplasia, cystic, susceptibility to = arT; renal dysplasia diffuse cystic =
arU; fibrochondrogenesi = aRu; ossifying fibroma = aru; giant cell fibroblastoma = aRU; deafness, autosomal dominant
13 = aRv; bruck syndrome = aRV; polycystic kidney - body part = arV; camurati-engelmann disease = arv; brittle bone disorder =
aRW; spinal muscular atrophy, lower extremity-predominant, 2, autosomal dominant = arW; pierre robin syndrome with
fetal chondrodysplasia = aRw; scleroatonic muscular dystrophy = arw; juvenile spinal muscular atrophy = arX; chondrodysplasia
punctata, x-linked dominant type = arx; lipodermatosclerosis = aRX; deafness, autosomal recessive 53 = aRx; peritoneal
mesothelioma = arY; chief cell adenocarcinoma = ary; megaepiphyseal dwarfism = aRy; collagen disease = aRY; stickler
syndrome, type 3 = aRz; caffey disease = aRZ; split-hand/foot malformation with long bone deficiency = arz; lesion of
stomach = arZ; vascular disease = aS; astrocytoma = as; aortic aneurysm and dissection = As; follicular adenoma = AS;
laron syndrome = asA; extranodal marginal zone b-cell lymphoma = asa; cervical incompetence = aSa; maxillofacial abnormalities =
aSA; familial amyloid polyneuropathy, type iv = asB; pseudosarcomatous fibromatosis = asb; aortic aneurysm, familial
abdominal 1 = aSB; sclerosteosis = aSb; autosomal recessive chronic granulomatous disease = asC; gastrointestinal lymphoma =
asc; ehlers-danlos syndrome, cardiac valvular form = aSc; cystocele = aSC; acrogeria = aSD; childhood malignant schwannoma =
asd; osteogenesis imperfecta with blue sclerae and normal teeth = aSd; brucellosis = asD; old thrombus = aSe; hiatus
hernia = aSE; bruton-type agammaglobulinemia = asE; intrinsic asthma = ase; alexander disease = asF; type iii ehlers-danlos
syndrome = aSF; stage iv skin melanoma = asf; fasciitis = aSf; melorheostosis = aSG; basal-like breast carcinoma = asG;
serum creatinine = aSg; degenerative myopia = asg; cervical polyp = asH; maturity-onset diabetes of the young, type 11 =
ash; platyspondylic lethal skeletal dysplasia, torrance type = aSh; rectal prolapse = aSH; stickler syndrome, type
i, nonsyndromeic ocular = aSi; haematotoxicity = asi; childhood medulloblastoma = asI; angiopathy, hereditary, with nephropathy,
aneurysms, and muscle cramps = aSI; chromosome instability syndromes = asj; osteogenesis imperfecta, type xiii = asJ;
stickler syndrome, type 1 = aSj; cerebral autosomal recessive arteriopathy with subcortical infarcts and leukoencephalyopathy =
aSJ; hepatitis, infectious canine = aSK; spondylometaphyseal dysplasia, kozlowski type = aSk; rothmund-thomson syndrome =
ask; pneumocystis infections = asK; epiphyseal dysplasia, multiple, with myopia and conductive deafness = aSl; irido-corneal
dysgenesis = aSL; mutagen sensitivity = asl; epidermaolysis bullosa dystrophyica = asL; trichorhinophalangeal syndrome
type ii = asM; osteoarthritis with mild chondrodysplasia = aSm; agammaglobulinemia 4, autosomal recessive = asm; paralytic
stroke = aSM; persistent generalized lymphadenopathy = asn; ovarian dysgenesis 2 = asN; spondyloperipheral dysplasia
short ulna = aSn; porencephaly 2 = aSN; complement component deficiency = aso; czech dysplasia, metatarsal type = aSo;
pierson syndrome = aSO; premature ovarian failure 4 = asO; parasitic helminthiasis infectious disease = asp; corneal
dystrophy, posterior polymorphous, 1 = aSP; jaw abnormalities = aSp; ovarian insufficiency = asP; alport syndrome, autosomal
recessive = aSQ; 46 xx gonadal dysgenesis = asQ; mast cell neoplasm = asq; rhegmatogenous retinal detachment, autosomal
dominant = aSq; benign mastocytoma = asr; hematuria, benign familial = aSR; ossification of the posterior longitudinal
ligament of the spine = asR; vitreoretinopathy with phalangeal epiphyseal dysplasia = aSr; cholesteatoma = asS; toxoplasmosis,
ocular = aSs; alport syndrome, autosomal dominant = aSS; chronic venous insufficiency = ass; trichorhinophalangeal syndrome =
aSt; trigeminal neuralgia = asT; biogenesis of lysosome-related organelles complex 1, subunit 3 = ast; alport syndrome,
recessive type = aST; bacterial osteomyelitis = asu; congenital infectious disease = aSu; root resorption = asU; benign
hematuria = aSU; hermansky-pudlak syndrome 9 = asv; avascular necrosis of the capital femoral epiphysis = aSv; chondroblastoma =
asV; retinal lattice degeneration = aSV; oculodentodigital dysplasia = asW; eye diseases, hereditary = aSW; emphysema,
hereditary pulmonary = asw; claudication = aSw; chronic idiopathyic pulmonary fibrosis = aSX; hyperbiliverdinemia = asx;
orofacial cleft 11 = asX; congenital vitreous anomaly = aSx; empty vitreous = aSy; axenfeld-rieger syndrome, type 3 =
asY; anterior lenticonus = aSY; pygmy = asy; nutrition disease = asz; microphthalmia, syndromeic 6 = asZ; antisynthetase
syndrome = aSZ; legg-calve-perthes disease = aSz; chronic obstructive pulmonary disease = at; mydriasis, congenital =
At; choroideremia = AT; breast cancer = aT; short syndrome = ata; sporadic primary pulmonary hypertension = atA; waardenburg's
syndrome = aTa; epiphyseal dysplasia, multiple, 6 = aTA; vascular occlusion = atB; acquired equinovarus deformity = aTB;
limb deformities, congenital = atb; esophagus leiomyoma = aTb; anorectal atresia = atc; deafness, x-linked 6 = aTc; myeloblastosis =
atC; epiphyseal dysplasia, multiple, 2 = aTC; stickler syndrome, type v = aTD; leiomyomatosis, esophageal and vulval,
with nephropathy = aTd; banti's syndrome = atD; aortic sclerosis = atd; multiple mitochondrial dysfunctions syndrome
2 = atE; chronic sciatica = aTE; kyphoscoliosis deformity of spine = aTe; aortic valve sclerosis = ate; dysmorphism =
atf; multiple mitochondrial dysfunctions syndrome = atF; corneal thinning = aTf; oculoskeletal dysplasia = aTF; epiphyseal
dysplasia, multiple, 3 = aTG; hypertrophyic obesity = atg; floppy = aTg; bisphosphoglycerate mutase = atG; cleft palate-lateral
synechia syndrome = ath; incontinentia pigmenti achromians = aTh; deficiency of bisphosphoglycerate mutase = atH; carnevale
syndrome = aTH; mucopolysaccharidosis i = ati; oculopalateoskeletal syndrome = aTI; keratosis pilaris = aTi; polycythemi =
atI; peptic esophagitis = atj; dengue shock syndrome = atJ; malpuech facial clefting syndrome = aTJ; myosclerosis,
autosomal recessive = aTj; holt-oram syndrome = atk; contagious pustular dermatitis = aTk; dog diseases = aTK; bacterial
pneumonia = atK; cirrhosis and chronic liver disease = aTL; benign scapuloperoneal muscular dystrophy with cardiomyopathy =
aTl; craniosynostosi = atL; hemochromatosis, type 2 = atl; polymyalgia rheumatica = atM; proximal weakness = aTm; sclerosteosi =
atm; epiphyseal dysplasia, multiple, 1 = aTM; pseudochondroplasia = aTN; andersen-tawil syndrome = atN; degenerative
spondylolisthesis = atn; epidermaolysis bullosa, pretibial = aln; renal artery occlusion = ato; pain, intractable = aTO;
transient bullous dermaolysis of the newborn = aTo; limb-girdle muscular dystrophy type 2a = atO; corneal fibrosis =
atp; epidermaolysis bullosa dystrophyica, pasini type = aTp; hearing loss, cisplatin-induced, susceptibility to = aTP;
nephrotic syndrome, type 3 = atP; noonan syndrome 7 = atQ; spondylolysis = atq; nociceptive pain = aTQ; epidermaolysis
bullosa pruriginosa = aTq; leopard syndrome 3 = atR; toenail dystrophy, isolated = aTr; spinal stenosis = atr; response
to pain = aTR; epidermaolysis bullosa with congenital localized absence of skin and deformity of nails = aTs; disorder
of endocrine ovary = aTS; diaphanospondylodysostosis = ats; abnormalities, multiple = atS; epidermaolysis bullosa dystrophyica
inversa, autosomal recessive = aTt; pain syndrome = aTT; neoplasms, multiple primary = atT; polyposis syndrome, hereditary
mixed, 2 = att; epidermaolysis bullosa dystrophyica, autosomal recessive, localisata variant = aTu; premenstrual dysphoric
disorder = aTU; skin/hair/eye pigmentation, variation in, 2 = atU; chromosome 10q23 deletion syndrome = atu; chondromalacia =
atv; dominant dystrophyic epidermaolysis bullosa = aTv; papillary microcarcinoma = atV; chronic tension-type headache =
aTV; generalized dystrophyic epidermaolysis bullosa = aTw; low grade serous carcinoma = atW; ebstein anomaly = atw;
maduromycosis = aTW; melanoma stage = atX; cushing's symphalangism = atx; acute postoperative pain = aTX; localized recessive
dystrophyic epidermaolysis bullosa = aTx; desmoplastic melanoma = atY; chondrodysplasia, acromesomelic, with genital
anomalies = aty; homicidal = aTY; epidermaolysis bullosa acquisita = aTy; pulmonary hypertension, primary, fenfluramine-associated =
atz; anxiety generalized = aTZ; serrated polyp = atZ; corneal dystrophy, posterior polymorphous, 2 = aTz; liposarcoma =
au; patent ductus arteriosus = Au; cryptogenic organizing pneumonia = AU; dystonia = aU; harderoporphyria = aUA; chronic
daily headache = aUa; nevus cell = aua; giant cell glioblastoma = auA; manic symptom = aUb; serrated adenocarcinoma =
aub; mucinous cystadenocarcinoma = auB; aplasia cutis congenita, reticulolinear, with microcephaly, facial dysmorphism,
and other congenital anomalies = aUB; familial multiple coagulation factor deficiency i = aUC; childhood anaplastic astrocytoma =
auc; stereotyped behavior = aUc; craniopharyngioma = auC; lobular panniculitis = aud; narcissism = aUd; papillary craniopharyngioma =
auD; von willebrand disease type 2n = aUD; acneiform dermatitis = auE; widespread disease = aue; stimulant dependencye =
aUe; von willebrand disease type 2m = aUE; familial nonmedullary thyroid cancer = auf; common bile duct neoplasm =
auF; dysfibrinogenemia = aUF; aggressive personality = aUf; prothrombin gene mutation = aUG; ovarian low malignant potential
tumorur = aug; maxillary neoplasm = auG; chronic low back pain = aUg; chronic pain syndrome = aUh; pleomorphic xanthoastrocytoma =
auH; mild hereditary factor viii deficiency disease = aUH; metastatic malignant peripheral nerve sheath tumor = auh;
free-floating anxiety = aUi; pilocytic astrocytoma of cerebellum = auI; eruptive melanocytic nevi = aui; moderate hereditary
factor viii deficiency disease = aUI; carrier of hereditary factor viii deficiency disease = aUJ; headache severe = aUj;
stage iii melanoma = auj; optic nerve astrocytoma = auJ; impulse control disorder = aUk; ovarian germ cell teratoma =
auK; endocrine carcinoma = auk; familial multiple factor deficiency syndrome, type i = aUK; acquired haemophilia = aUL;
urachal adenocarcinoma = aul; malignant struma ovarii = auL; ocular motility disease = aUl; perianal fistula = aum; severe
hereditary factor viii deficiency disease without inhibitor = aUM; lung adenoma = auM; eumycotic mycetoma = aUm; speech
disorder = aUn; malignant choroid melanoma = auN; irreversible pulpitis = aUN; infratentorial cancer = aun; multiple
system atrophy 1, susceptibility to = aUo; follicular dendritic cell sarcoma = auO; pseudotumor cerebri = aUO; cerebellum
cancer = auo; central retinal veins occlusion = aUP; metanephric adenoma = auP; articulation disorder = aup; coenzyme
q10 deficiency, primary, 6 = aUp; common cold = auq; hyperferritinemia, hereditary, with congenital cataracts = aUQ;
malignant iris melanoma = auQ; coenzyme q10 deficiency, primary, 5 = aUq; frontal lobe neoplasm = aur; hcl-v = auR; benign
essential blepharospasm = aUR; electron transport chain deficiencyies, mitochondrial = aUr; muir-torre syndrome = auS;
hepatitis, animal = aUS; syndactyly cenani lenz type = aUs; nodular goiter = aus; cryptosporidiosis = auT; pasteurellaceae
infections = aUT; sclerosteosis 2 = aUt; alexia = aut; preeclampsia/eclampsia 5 = aUu; rigidity and multifocal seizure
syndrome, lethal neonatal = auU; familial apoceruloplasmin deficiency = aUU; isovaleric acidemia = auu; riddle syndrome =
auV; basal ganglion degeneration = aUV; polysyndactyly = aUv; panniculitis = auv; iron metabolism disease = aUW; conjunctival
cancer = auw; immunodeficiency 8 = aUw; breast cancer 3 = auW; hemosiderosis = aUX; li-fraumeni-like syndrome = auX;
leigh syndrome due to mitochondrial complex iv deficiency = aUx; malignant conjunctiva melanoma = aux; cardioencephalyomyopathy,
fatal infantile, due to cytochrome c oxidase deficiency 2 = aUy; high grade serous carcinoma = auY; infarct of liver =
aUY; mandibular cancer = auy; breast cancer stage = auZ; blepharospasm = aUZ; exocrine pancreatic insufficiency, dyserythropoietic
anemia, and calvarial hyperostosis = aUz; struma ovarii = auz; lung benign neoplasm = av; hereditary spastic paraplegia =
aV; subfertility = AV; loeys-dietz syndrome = Av; ocular cancer = avA; febrile seizures, familial, 11 = aVa; atypical
medullary carcinoma = ava; poikiloderma with neutropenia = aVA; occult malignancy = avb; floating-harbor syndrome = aVB;
tumor of exocrine pancreas = avB; epilepsy, familial temporal lobe, 5 = aVb; pulmonary thromboembolism = aVc; ovarian
carcinosarcoma = avC; chromosome 16p13.3 deletion syndrome = aVC; breast cancer, familial male = avc; lupus erythematodes =
aVD; breast secretory carcinoma = avD; hyperproinsulinemia = aVd; pelvic cancer = avd; carboxypeptidase n deficiency =
aVe; neonatal leukemia = aVE; spinal cord primitive neuroectodermal neoplasm = avE; occult carcinoma = ave; sexual
precocity = aVF; curb = avf; antley-bixler syndrome = avF; hereditary coproporphyria = aVf; cleft palate x-linked = aVg;
atrioventricular septal defect, partial, with heterotaxy syndrome = aVG; lobular carcinoma in situ = avg; severe anemia =
avG; atrioventricular septal defect, susceptibility to, 2 = aVH; cleft palate with ankyloglossia = aVh; loss of interest =
avh; adrenal hyperplasia = avH; nonproliferative fibrocystic disease = avi; complete atrioventricular septal defect =
aVI; coproporphyria = aVi; idiopathyic epilepsy = avI; atrioventricular septal defect: atrial and ventricular components =
aVJ; hypergonadotropic amenorrhea = avJ; infection due to brucella suis = aVj; difficulty sleeping = avj; malignant
neoplasm of abdomen = avk; maturation defect = aVK; chromosome 3, monosomy 3p = avK; erythropoietic protoporphyri = aVk;
obesity in adolescence = avl; la crosse encephalyitis = aVl; cystic ovaries = avL; acth deficiency, isolated = aVL; psychophysiologic
disorders = aVM; breast carcinoma in situ = avm; testicular dysfunction = avM; malaria, severe, resistance to = aVm;
fecal peritonitis = aVN; mouth disease = avn; gonadotrophy adenoma = avN; proliferative nephritis unspecified = aVn;
hyperprolactinemia = avO; dysplastic nevus syndrome = avo; systemic lupus erythematosus, susceptibility to, 9 = aVo;
sarcoid arthritis = aVO; female reproductive organ cancer = avp; pituitary apoplexy = aVP; immunodeficiency, common variable,
7 = aVp; panhypopituitarism = avP; charge syndrome = avQ; hypothalamic disease = aVQ; uveal cancer = avq; oral hairy
leukoplaki = aVq; fallopian tube carcinoma = avr; dyspnea, paroxysmal = aVR; mental retardation, autosomal recessive
34 = aVr; perry syndrome = avR; fallopian tube cancer = avs; functional colonic disease = aVS; retinitis pigmentosa 12 =
aVs; hereditary persistence of fetal hemoglobin thalassemia = avS; stuve-wiedemann syndrome = aVT; leber congenital
amaurosis 8 = aVt; stinging sensation = avT; microglandular adenosis = avt; familiar ovarian carcinoma = avu; nut midline
carcinoma = avU; kaposi varicelliform eruption = aVU; anetoderma = aVu; xeroderma pigmentosum, group e = aVV; slow acetylator
due to n-acetyltransferase enzyme variant = avV; microglandular adenosis of breast = avv; hyperopia = aVv; inflammatory
disorder of breast = aVW; incisional hernia = avW; retinal telangiectasisa = aVw; fanconi anemia, complementation group
di = avw; acute uveitis = aVX; pituitary dwarfism 1 = avx; mental retardation, autosomal recessive 2 = aVx; cerebral
salt-wasting syndrome = avX; cocaine use = aVy; chorioretinitis = avY; pneumococcal bacteraemia = aVY; breast-ovarian
cancer, familial, susceptibility to, 2 = avy; vulva squamous cell carcinoma = avZ; bacteremia due to staphylococcus aureus =
aVZ; progressive aphasia = avz; small cell osteogenic sarcoma = aVz; juvenile-onset dystonia = Aw; leprosy = AW; myocardial
infarction = aw; breast carcinoma = aW; dentinogenesis imperfecta = awa; sickle cell-beta-thalassemia = aWa; premature
chromatid separation trait = awA; cataract, congenital, cerulean type, 2 = aWA; chronic peri-aortitis = aWb; anauxetic
dysplasia = awB; cataract, congenital nuclear, autosomal recessive 2 = aWB; dentin dysplasia = awb; legionnaires' disease =
aWc; fanconi anemia, complementation group j = awc; cataract 39, multiple types = aWC; mean corpuscular hemoglobin =
awC; nose disease = aWd; cataract, punctate, progressive juvenile-onset = aWD; fanconi syndrome = awd; fatty acid oxidation
defects = awD; angiomas = awe; albinism, oculocutaneous, type vii = awE; cataract, crystalline aculeiform = aWE; paratyphoid
fever = aWe; progressive external ophthalmoplegia with mitochondrial dna deletions, autosomal dominant, 3 = awF; seronegative
arthritis = aWF; mental retardation, x-linked 93 = awf; septic arthritis = aWf; senile cataract = aWG; hereditary motor
neuronopathy = awg; osteogenesis imperfecta type vii = aWg; infantile onset spinocerebellar ataxia = awG; osteogenesis
imperfecta, type 7 = aWh; sensory ataxiac neuropathy, dysarthria, and ophthalmoparesis = awH; cataract 20, multiple types =
aWH; blood group--ok = awh; pseudoaphakia = aWI; eccrine acrospiroma = aWi; chikungunya fever = awi; mitochondrial
dna depletion syndrome 3 (hepatocerebral type) = awI; delayed emergence from anesthesia = awj; parkinson disease, mitochondrial =
awJ; clear cell hidradenoma = aWj; giardiasis = aWJ; flea infestations = aWK; leber congenital amaurosis 7 = aWk; restless
legs syndrome, susceptibility to, 6 = awk; optic atrophy = awK; chronic progressive external ophthalmoplegia = awL; spotted
fever = aWL; cone-rod dystrophy 2 = aWl; attention-deficit/hyperactivity disorder, predominantly inattentive type = awl;
periodic limb movement disorder = awm; tay-sachs disease = awM; heroin dependency = aWm; typhus = aWM; melas syndrome =
awN; biotin deficiency = awn; myopathy, myofibrillar, desmin-related = aWn; hyperexplexia hereditary = aWN; gliosis,
familial progressive subcortical = aWO; multiple carboxylase deficiency = awo; alpha-b crystallinopathy = aWo; chondroid
lipoma = awO; benign ependymom = awP; hereditary diffuse leukoencephalyopathy with spheroids = aWP; holocarboxylase synthetase
deficiency = awp; cardiomyopathy, dilated, iii = aWp; cataract, posterior polar, 2 = aWq; protein-energy malnutrition =
awq; localized vitisligo = awQ; fibrosisng adenosis = aWQ; thymic carcinoma = awr; sclerosisng adenosis of breast =
aWR; temtamy syndrome = awR; cataract 16, multiple types = aWr; type c thymoma = aws; neutrophyilia, hereditary = aWS;
disorder of skeletal muscle = aWs; microphthalmia and mental deficiency = awS; congenital posterior polar cataract =
aWt; hypogammaglobulinemia and isolated growth hormone deficiency, x-linked = awt; combined oxidative phosphorylation
deficiency 7 = awT; dystonia, dopa-responsive, due to sepiapterin reductase deficiency = aWT; band-like calcification
with simplified gyration and polymicrogyria = aWU; hypogammaglobulinemia, x-linked = awu; cataract, congenital zonular,
with sutural opacities = aWu; spastic paraplegia 55, autosomal recessive = awU; cataract 23 = aWv; recurrent bacterial
infection = aw; abdominal neoplasms = awV; surfactant metabolism dysfunction, pulmonary, 1 = aWV; anemia, congenital
dyserythropoietic, type ib = awW; pyoderma = aww; cataract, lamellar 2 = aWw; surfactant metabolism dysfunction, pulmonary,
4 = aWW; segawa syndrome, autosomal recessive = aWX; rubell = awx; hydronephrosi = awX; cataract, congenital nuclear,
autosomal recessive 3 = aWx; cataract microcornea syndrome = aWy; sarcoidosis, susceptibility to, 2 = awy; purpura fulminans =
aWY; prostatic adenoma = awY; neurodegeneration with brain iron accumulation 4 = awZ; bone marrow failure syndromes =
aWZ; lofgrens syndrome = awz; cataract, coppock-like = aWz; cardiomyopathy, familial restrictive, 1 = AX; iris coloboma
with ptosis, hypertelorism, and mental retardation = Ax; adrenal cortical hypofunction = ax; early disease onset = ax;
follicular cyst = aXa; fatal familial insomni = axA; severe beta thalassemia = aXA; neurodegeneration with brain iron
accumulation (nbia) = axa; amelogenesis imperfecta, hypomaturation type, iia4 = axB; spastic paraplegia 43, autosomal
recessive = axb; eosinophilia, familial = aXb; clinical chorioamnionitis = aXB; wolf-hirschhorn syndrome = axC; selenium
deficiency = axc; chronic neutrophyilia = aXC; staphylococcal skin infections = aXc; lepromatous leprosy = axd; primary
ulcer of intestine = aXD; protein intolerance = aXd; gonococcal arthritis dermatitis syndrome = axD; ectopic tooth =
aXE; acute flaccid paralysis = aXe; hypersensitivity reaction type iii disease = axE; retrograde degeneration = axe;
lens disease = axf; transient myocardial ischemia = axF; splenic infarction = aXF; meningitis/encephalyitis = aXf; acute
necrotizing myelitis = aXg; complement 1s deficiency = axg; pericardial mesothelioma = aXG; bronchopneumonia = axG; joubert
syndrome 17 = axH; cockayne syndrome a = aXH; homocarnosinase deficiency = aXh; complement component cls deficiency =
axh; myeloradiculopathy = aXi; amyotrophy, monomelic = axl; breast cancer and pregnancy = aXI; macular degeneration,
age-related, 14 = axi; pulmonary eosinophilic granuloma = aXj; complement component 6 deficiency = axJ; complement deficiency =
axj; isolated growth hormone deficiency, type ii = aXJ; 15q24 microdeletion = aXK; periadenitis mucosa necrotica recurrens =
aXk; c6 deficiency, subtotal = axK; intracranial glioma = axk; complement component 7 deficiency = axL; parietal lobe
neoplasm = aXL; ectopic pancreas = axl; stromal keratitis = aXl; advanced sleep phase syndrome, familial, 2 = aXM; ciliary
dyskinesia, primary, 26 = axm; cone-rod dystrophy 16 = axM; mumps meningitis = aXm; multiple sclerosis in children =
aXn; macular degeneration, age-related, 9 = axn; c9 deficiency = axN; advanced sleep-phase syndrome, familial = aXN;
hemolytic uremic syndrome, atypical, susceptibility to, 5 = axo; hib meningitis = aXo; macular degeneration, age-related,
15 = axO; advanced sleep phase = aX0; brainstem encephalyitis = aXp; pneumonia, pneumococcal = axp; facial dermatosis =
axP; classical glioblastoma = aXP; complement component 3 deficiency, autosomal recessive = axq; joubert syndrome 21 =
aXQ; polycystic lipomembranous osteodysplasia with sclerosisng leukoencephalyopathy = axQ; enterovirus meningitis =
aXq; drug exanthema = aXr; frontotemporal dementia with motor neuron disease = axR; immune complex nephritis = axr; cardiomyopathy,
dilated, 1 m = aXR; cardiomyopathy, familial hypertrophyic, 12 = aXS; anterior uveitis = axs; familial dementia = axS;
lymphohistiocytosis = aXs; complement component 3 deficiency = axt; hereditary cystatin c amyloid angiopathy = aXT; eyelid
disease = aXt; amyotrophyic lateral sclerosis with dementia = axT; hereditary cerebral amyloid angiopathy, icelandic
type = aXU; pemphigus = axu; intermittent explosive disorder = aXu; huntington disease-like syndrome = axU; progressive
muscular atrophy = axV; intracranial hypotension = aXv; methemoglobinemia, type i = axv; macular degeneration, age-related,
11 = aXV; papillon-lefevre disease = axW; ectopic pregnanc-aXw; aXw; complement component 4a deficiency = axw; amyloidosis,
hereditary = aXW; salt-losing congenital adrenal hyperplasia = axx; surfactant metabolism dysfunction, pulmonary, 5 =
aXx; hyperchlorhidrosis, isolated = axX; sporadic cerebral amyloid angiopathy = aXX; somatosensory disorders = aXy; niemann-pick
disease, type c2 = aXY; glaucoma both eyes = axY; adrenal incidentaloma = axy; complement component 4b deficiency = axz;
exfoliative ichthyosis, autosomal recessive, ichthyosis bullosa of siemens-like = aXZ; neutropenia, severe chronic =
aXz; non-specific brain syndrome = axZ; cardiomyopathy, familial hypertrophyic, 1 = AY; paraneoplasmtic pemphigus = ay;
kidney failure = aY; familial hypophosphatemiac rickets = Ay; mucolipidosis type i = aYa; nutritional deficiency disease =
aya; blood autoimmune disorders = aYA; x-linked csnb = ayA; action myoclonus = aYb; astigmatism = ayB; intermediate
uveitis = aYB; retinitis pigmentosa 17 = ayb; basal ganglia calcification = aYc; epilepsy, childhood absence, susceptibility
to, 6 = ayC; arrhythmogenic right ventricular dysplasia, familial, 13 = aYC; carbonic anhydrase va deficiency, hyperammonemia
due to = ayc; epilepsy, idiopathyic generalized, susceptibility to, 6 = ayD; trichohepatoenteric syndrome = aYD; cerebroretinal
microangiopathy with calcifications and cysts = aYd; decayed, missing and filled tooth count = ayd; mental retardation,
autosomal dominant 19 = aYE; gastric antral vascular ectasisa = aYe; malignant hyperthermia susceptibility type 5 = ayE;
xerophthalmia = aye; thyrotoxic periodic paralysis, susceptibility to, 1 = ayF; hepatitis b and hepatitis c = aYF; cockayne
syndrome b = aYf; cerebellar ataxia, mental retardation, and dysequilibrium syndrome 3 = ayf; adamantinous craniopharyngioma =
aYG; neurilemmomatosis = ayg; malignant hyperthermia, susceptibility to, 5 = ayG; mental retardation, autosomal dominant
21 = aYg; periodic paralysis = ayH; deafness, autosomal recessive 93 = ayh; leukemia, megakaryoblastic, of down syndrome =
aYh; classic pulmonary blastoma = aYH; familial periodic paralysis = ayI; small intestine carcinoma = aYI; night blindness,
congenital stationary, type 2b = ayi; sibling jealousy = aYi; epilepsy, idiopathyic generalized, susceptibility to, 11 =
ayJ; night blindness = ayj; congenital cataracts, facial dysmorphism, and neuropathy = aYj; cri-du-chat syndrome =
aYJ; zimmerman laband syndrome = ayK; pulmonary congestion = aYk; episodic ataxia, type 1 = ayk; cystinosis, late-onset
juvenile or adolescent nephropathyic type = aYK; cystinosis, ocular nonnephropathyic = aYL; hyperosmolality = aYl; chronic
back pain = ayL; spinocerebellar ataxia 26 = ayl; episodic ataxia, type 7 = aym; retinal cone dystrophy 4 = ayM; severe
persistent asthma = aYm; cystinosis, atypical nephropathyic (disorder) = aYM; brugada syndrome 4 = ayN; cystinosis, infantile
nephropathyic = aYN; torticollis = ayn; skin amelanotic melanoma = aYn; restrictive cardiomyopathy = aYo; crossed hemiplegia =
ayo; episodic ataxia, type 5 = ayO; pancreatitis, chronic, susceptibility to = aYO; neuraminidase deficiency with beta-galactosidase
deficiency = aYP; mental retardation, autosomal dominant 10 = ayP; familial migraine = ayp; gingival fibromatosis = aYp;
diabetes mellitus, insulin-dependent, 12 = aYq; neuraminidase 1 deficiency = aYQ; spell = ayq; familial paroxysmal dystonia =
ayQ; gangliosidosis gml = aYR; timothy syndrome = ayr; celiac disease, susceptibility to, 3 = aYr; disease of mental
healty = ayR; hypothyroidism, autoimmune = aYs; brugada syndrome 3 = ays; cancer relapse = ayS; pycnodysostosis = aYS;
keratolytic winter erythema = aYT; microscopic polyangiitis = aYt; high grade cervical squamous intraepithelial neoplasmia =
ayT; feeling powerless = ayt; primary aldosteronism, seizures, and neurologic abnormalities = ayu; cervical cancer
squamous cell = ayU; glycosphingolipidoses = aYU; pretibial myxedema = aYu; sinoatrial node dysfunction and deafness =
ayv; purulent sputum = aYV; autoimmune response = aYv; complicated pneumoconiosis = ayV; clonorchiasis = aYW; familial
mediaterranean feve = ayW; aland island eye disease = ayw; thyroid associated orbitopathy = aYw; chronic chagas' disease =
aYx; fascioliasis = aYX; adenofibroma = ayX; cone-rod dystrophy 3 = ayx; cone-rod dystrophy, x-linked, 3 = ayy; keratosis
palmoplantaris with periodontopathyia and onychogryposis = aYY; deficiency of vitamin d3 = aYy; sarcomatoid mesothelioma =
ayY; atrophy of optic disc = ayz; tropical calcific pancreatitis = ayZ; early onset periodontitis = aYZ; ulcerative
jejunitis = aYz; cardiomyopathy, familial hypertrophyic, 11 = AZ; helicobacter pylori [h. pylori] = az; craniofacial
abnormalities = Az; ovarian disease = aZ; foot ulcer = aza; agoraphobie= azA; myh9-related disorders = aZA; deficiency
of cathepsin c = aZa; mass of thyroid gland = azb; subacromial bursitis = aZB; hypotonia-cystinuria syndrome = azB; ceroid
lipofuscinosis, neuronal, 10 = aZb; visually threatening diabetic retinopathy = aZC; c cell tumor = azc; neuronal ceroid
lipofuscinosis, congenital = aZc; isolated cystinuria = azC; postpartum cardiomyopathy = aZd; neutropenia chronic = azD;
pain during injection = azd; bursitis = aZD; subgingival plaque = azE; tertiary neurosyphilis = aZE; hypoalgesia = aze;
ceroid lipofuscinosis, neuronal, 13 = aZe; rosacea = azF; platelet thrombus = aZf; lung large cell carcinoma = azf; gastric
diffuse adenocarcinoma = aZF; hyperparathyroidism = azg; dyggve-melchior-clausen syndrome = aZG; cerebellar ataxia, nonprogressive,
with mental retardation = azG; sprains and strains = aZg; epithelioid hemangioendothelioma = azH; hypercalcemia = azh;
secondary anemia = aZh; aspiration pneumoniatis = aZH; cherubism = azi; desbuquois dysplasia = azI; aspiration pneumonia =
aZI; light chain myeloma = aZi; peritonsillar abscess = aZJ; pseudohypoaldosteronism, type iie = aZj; supravalvular
aortic stenosis = azj; diabetes mellitus, noninsulin-dependent, 1 = azJ; diarrhea persistent = aZK; tibial muscular dystrophy,
tardive = azK; femoral neck fractures = azk; mental retardation, x-linked, with brachydactyly and macroglossia = aZk;
aggressive outburst = aZl; diffuse panbronchiolitis = aZL; alpha-sarcoglycanopathyies = azL; acute closed-angle glaucoma =
azl; chronic mesangial proliferative glomerulonephritis = aZm; arthritis, reactive = azm; lichenoid eruptions = aZM;
myositis, eosinophilic = azM; periapical diseases = aZn; rhinosinusitis = aZN; residual cancer = azn; immunodeficiency
11 = azN; adnexal diseases = aZO; cutaneous hypersensitivity = aZo; whooping cough = azo; psoriasis susceptibility 2 =
azO; dysentery, bacillary = aZP; familial psoriasis = azP; floppy lid syndrome = azp; leukocytoclastic vasculitis =
aZp; histoplasmosis = azq; small fibre neuropathy = aZQ; macular degeneration, age-related, 12 = aZq; familial pityriasis
rubra pilaris = azQ; headache recurrent = aZr; chronic secretory otitis media = aZR; pityriasis rubra pilaris = azR;
hemangiopericytoma = azr; orofacial cleft = azs; intestinal obstruction = aZs; first myocardial infarction = azS; fertility
disorders = aZS; thyroid cancer metastatic = aZT; thickening of pleura = azT; neonatal necrotizing enterocolitis = aZt;
oculocutaneous albinism type 1 = azt; listeriosis = azU; insulin resistance - type a = azu; rapidly progressive periodontitis =
aZU; adenoviral keratitis = aZu; prodromal stage = aZV; acute hepatitis c = aZv; candidiasis familial chronic mucocutaneous,
autosomal recessive = azV; surrogate mother = azv; nose symptoms = aZW; erythema migrans = aZw; candidemia = azW; ampulla
of vater neoplasm = azw; dermatophytosis = azX; onchocerciasis = azx; diarrhea in children = aZX; pulpitis = aZx; clostridium
difficile diarrhoea = aZY; scarlet fever = azy; invasive aspergillosis = azY; geographic tongue = aZy; bilis = aZZ; periapical
granuloma = aZz; microcephaly, primary autosomal recessive, 4 = azZ; cardiomyopathy, familial hypertrophyic, 19 = azz;
spots on skin = b; carcinoma, hepatocellular = B; osteoporosis = bA; gingival disease = ba; atrial septal defect 5 =
Ba; pancreatic ductal carcinoma = BA; allergic symptom = baA; spastic paraplegia 46, autosomal recessive = bAa; incurable
diseases = baa; cataract, congenital or juvenile = bAA; familial cardiomyopathy = bAB; eosinophilic syndrome = bab; methemoglobinemia
type iv = baB; polyglucosan body disease, adult form = bAb; recurrent sinusitis = bac; gsd iv, neuromuscular form, congenital =
bAc; 17,20-lyase deficiency, isolated = baC; adult i blood group phenotype = bAC; seropositive rheumatoid arthritis =
bAD; staphylococcus aureus endocarditis = bad; urinary symptoms = bAd; hemoglobin m disease = baD; autosomal recessive
scid = bAE; nadh cytochrome b5 reductase deficiency = baE; glycogen storage disease iv = bAe; gastric ulcer benign =
bae; methemoglobinemia, type ii = baF; cells in urine = bAF; polymyalgia rheumatic = bAf; salpingitis = baf; isocyanate
induced asthma = bAG; bullous keratopathy = bag; granulomatous disease, chronic, autosomal recessive, cytochrome b-negative =
baG; diabetes mellitus, type ii, autosomal dominant = bAg; noninsulin-dependent diabetes mellitus with deafness = bAh;
ichthyosis exfoliativa = bAH; granulomatous disease, chronic, x-linked = baH; bronchial disease = bah; atypical mycobacteriosis,
familial, x-linked 2 = baI; reactive hypoglycemia = bAi; splenic sequestration = bai; autosomal dominant ichthyosis =
bAI; zap70 deficiency = baJ; protein-deficient diabetes mellitus = bAJ; newborn respiratory distress syndrome = baj;
oculopharyngodistal myopathy = bAj; dhpr deficiency = bAk; acute pancreatitiss = bak; colon (non-specific) inflammation =
baK; alcohol myopathy = bAK; chronic granulomatous disease, type ii = baL; cecum cancer = bAL; hyperphenylalaninemia,
bh4-deficient, b = bAl; pancreatitiss = bal; dystonia 6, torsion = bAm; rem sleep behavior disorder = bAM; mitochondrial
complex iii deficiency, nuclear type 6 = baM; hand dermatosis = bam; thrombocytopenia 4 = baN; juvenile parkinson's disease =
bAn; pediatric osteosarcoma = ban; hyperglycinemia, transient neonatal = bAN; lung giant cell carcinoma = bao; suicidal
ideation = bAO; beta thalassemia, heterozygous = baO; childhood onset dystonias = bAo; clostridium difficile colitis =
bap; charcot-marie-tooth disease, type 4a = bAP; mechanical pain = bAp; nonepidermaolytic palmoplantar keratoderma =
baP; cystitis and urethritis = baq; eccrine dermal cylindroma = baQ; charcot-marie-tooth disease, type 2k = bAQ; oromandibular
dystonia = bAq; secondary dystonia = bAr; pneumonia due to escherichia coli = baR; acute cystitis = bar; charcot-marie-tooth
disease, type 4a, axonal form = bAR; mastitis = bas; transmissible mink encephalyopathy (tme) = baS; charcot-marie-tooth
disease, recessive intermediate a = bAS; lumbar radiculopathy = bAs; pleural plaque = bat; transposition of the great
arteries, dextro-looped 3 = bAT; short bowel syndrome = baT; vestibulodynia = bAt; telangiectasisa, hereditary hemorrhagic,
type 5 = bAU; healing ulcer = bau; spiradenoma = baU; hyperinsulinemiac hypoglycemia, familial, 3 = bAu; renal cancer
metastatic = bav; lipoid congenital adrenal hyperplasia = baV; diabetes in youth = bAv; klippel feil syndrome dominant
type = bAV; klippel-feil syndrome 3, autosomal dominant = bAW; hyperviscosity syndrome = baw; adrenal insufficiency,
congenital, with 46,xy sex reversal, partial or complete = baW; hypoglycaemic episode = bAw; microphthalmia, isolated
7 = bAX; adrenal insufficiency, congenital = baX; gamma-glutamylcysteine synthetase deficiency, hemolytic anemia due
to = bAx; painful bladder syndrome = bax; congenital adrenal hyperplasia due to 11-beta-hydroxylase deficiency = baY;
mycobacterium avium infection = bay; coronary vessel anomalies = bAy; microphthalmia, isolated, with coloboma 6 = bAY;
klippel-feil syndrome = bAZ; hypoparathyroidism, autosomal recessive = bAz; familial testotoxicosis = baZ; t lymphoblastic
leukemia/lymphoma = baz; cardiomyopathy, dilated, 1r = Bb; galloway-mowat syndrome = BB; colon cancer = bB; pick's disease =
bb; hemorrhoid = bbA; chondrodysplasia, grebe type = bBa; 18-hydroxylase deficiency = bba; myasthenic syndrome, congenital,
with tubular aggregates 1 = bBA; acromesomelic dysplasia, maroteaux type = bBb; sporadic porphyria cutanea tarda = bbB;
corticosterone methyloxidase type ii deficiency = bbb; myasthenia, limb-girdle, with tubular aggregates = bBB; lingual-facial-buccal
dyskinesi = bBC; fibular hypoplasia and complex brachydactyly = bBc; sprays = bbC; 11-beta-hydroxylase deficiency = bbc;
deficiency of monooxygenase = bbd; multiple synostoses syndrome 2 = bBd; pseudoxanthoma elasticum-like disorder with
multiple coagulation factor deficiency = bBD; endometriosis of pelvis = bbD; glaucoma 1, open angle, a = bbE; familial
hyperaldosteronism = bbe; vertical talus, congenital = bBe; pseudoxanthoma elasticu = bBE; familial hyperaldosteronism
type 1 = bbf; brachydactyly, type al, c = bBf; glaucoma 3, primary congenital, a = bbF; glutathionuria = bBF; aldosterone
to renin ratio, increased = bbg; glaucoma of childhood = bbG; radial-renal syndrome = bBG; acromesomelic dysplasia =
bBg; aldosterone hypertension = bbh; symphalangism, proximal, 1b = bBh; early-onset glaucoma = bbH; tropical eosinophilia =
bBH; hyperreninemiac hypoaldosteronism = bbi; infection by wuchereria bancrofti = bBI; hyperandrogenism, nonclassic
type, due to 21-hydroxylase deficiency = bbI; microphthalmia, isolated 4 = bBi; leber congenital amaurosis 17 = bBj;
late-onset congenital adrenal hyperplasia = bbJ; high renin hypertension = bbj; pituitary hormone deficiency, combined,
2 = bBJ; spondylocostal dysostosis 4, autosomal dominant = bBk; adrenal hyperplasia, congenital, type 5 = bbk; mineralocorticoid
deficiency = bbK; isolated growth hormone deficiency, type ib = bBK; kowarski syndrome = bBL; gdp dissociation inhibitor
1 = bBl; deficiency of lyase = bbl; mild steroid 21-hydroxylase deficiency = bbL; short stature, idiopathyic, autosomal =
bBM; growth control, y-chromosome influenced = bbm; hypercalcemia, idiopathyic, of infancy = bbM; mental retardation,
x-linked 3 = bBm; genitalia external ambiguous = bbn; insulin-like growth factor i deficiency = bBN; hirschsprung disease,
susceptibility to, 3 = bBn; osteomalacia secondary to drug = bbN; sexual infantilism = bbo; hirschsprung disease type
3 = bBo; idiopathyic infantile hypercalcemia - mild form = bbO; secondary hypothyroidism = bBO; secondary parkinson disease =
bBp; dysmetabolic syndrome = bbp; adenocarcinoma of liver = bbP; tumor-induced hypoglycemia = bBP; birdshot chorioretinopathy =
bBq; radiohumeral fusions with other skeletal and craniofacial anomalies = bbQ; coenuriasis = bbq; potassium depletion =
bBQ; keratoconjunctivitis = bBR; urination disorders = bBr; estrogen excess = bbr; focal facial dermal dysplasia 4 =
bbR; dementia of frontal lobe type = bbS; leydig cell tumor = bbs; gemistocytic astrocytoma = bBs; pituitary hypoplasia =
bBS; cerebrotendinous xanthomatosis = bbT; small for gestational age = bBT; myopathy, mitochondrial progressive, with
congenital cataract, hearing loss, and developmental delay = bBt; froelich's syndrome = bbt; prognathism = bBU; polyp
of corpus uteri = bbu; carbon tetrachloride poisoning = bBu; vitamin d hydroxylation-deficient rickets, type 1a = bbU;
vitamin d-dependent rickets, type 2a = bbV; neutropenia, severe congenital, autosomal dominant 2 = bBv; environmental
illness = bbv; hypothyroidism, congenital, nongoitrous, 3 = bBV; atrichia with papular lesions = bbW; aryl hydrocarbon
hydroxylase inducibility = bbw; neutropenia, nonimmune chronic idiopathyic, adult = bBw; somatotrophy hyperplasia = bBW;
constitutional delay of growth and puberty = bBX; thrombasthenia-thrombocytopenia, hereditary = bBx; hidradenitis suppurativa =
bbx; rickets, hereditary vitamin d-resistant = bbX; cystadenocarcinoma = bby; combined oxidative phosphorylation deficiency
1 = bBy; tsh secreting adenoma = bBY; primary osteoporosis = bbY; acute lower respiratory tract infection = bbZ; oxidative
phosphorylation deficiencyies = bBz; gastric cardia carcinoma = bbz; congenital deficiency of intrinsic factor = bBZ;
ataxia telangiectasisa = bC; primary biliary cirrhosis = bc; myocardial reperfusion injury = Bc; spinocerebellar ataxia
5 = BC; congenital sensorineural hearing loss = bCA; normal bowel habits = bCa; atrichia = bca; debrisoquine, ultrarapid
metabolism of = bcA; parkinson disease 11 = bCb; postoperative vomiting = bcB; multidrug-resistant tuberculosis = bcb;
hearing impaired children = bCB; coumarin resistance = bcc; deafness, autosomal recessive 15 = bCc; pseudoparkinsonism =
bcC; hearing loss associated with syndrome = bCC; sensorineural hearing los = bCd; basal ganglia disease = bcD; eccrine
nevus = bCD; liver diseases, parasitic = bed; follicular occlusion triad - hidradenitis, acne conglobata, dissecting
cellulitis of scalp = bCE; lung cancer, protection against = bce; limited scleroderma = bcE; syndactyly, type 3 = bCe;
tegafur, poor metabolism of = bcf; atrioventricular septal defect 3 = bCf; mutism = bCF; alcoholic liver damage = bcF;
halothane hepatitis = bcG; oculodentodigital dysplasia, autosomal recessive = bCg; infestation = bcg; charcot-marie-tooth
disease type 2 = bCG; cirrhosis - non-alcoholic = bcH; opisthorchiasis = bch; deafness, autosomal dominant 3b = bCH;
craniometaphyseal dysplasia, autosomal recessive type = bCh; bietti crystalline dystrophy = bci; crisscross heart = bCi;
deafness, autosomal recessive 1b = bCI; vitamin d hydroxylation-deficient rickets, type b = bcI; congenital hearing disorder =
bCJ; spastic paraplegia 56, autosomal recessive = bcJ; lamellar ichthyosis, type 3 = bcj; hallermann-streiff syndrome =
bCj; deafness, digenic, gjb2/gjb6 (disorder) = bCK; cataract, zonular pulverulent 3 = bCk; efavirenz, poor metabolism
of = bcK; deficiency of isomerase = bck; cataract, zonular pulverulent 1 = bCl; leukodystrophy, hypomyelinating, 2 =
bCL; drug-induced hepatitis = bcl; arteriolar hyalinosis = bcL; food-drug interactions = bcm; axonal sensorimotor neuropathy =
bCm; ichthyosis, nonlamellar and nonerythrodermaic, congenital, autosomal recessive = bcM; lymphedema, hereditary,
ic = bCM; hepatic impairment = bcn; torulopsis = bcN; spastic paraplegia 44, autosomal recessive = bCN; cns symptom =
bCn; chromosome xp21 deletion syndrome = bCO; vegetation = bcO; charcot-marie-tooth disease type = bCo; drug metabolism,
poor, cyp2c19-related = bco; neurological pain = bCP; mephenytoin, poor metabolism of = bcp; neuromyelitis optic = bcP;
deafness, autosomal recessive 1a = bCp; acroparesthesia = bCQ; vohwinkel syndrome = bCq; premenstrual syndrome = bcq;
spastic paraplegia 5a, autosomal recessive = bcQ; spastic paraplegia type 5a, recessive = bcR; cornea verticillata =
bCR; knuckle pads, leuconychia and sensorineural deafness = bCr; rhabdomyolysis, cerivastatin-induced = bcr; jaw diseases =
bcs; keratoderma palmoplantar deafness = bCs; pseudo-hurler polydystrophy = bCS; autosomal recessive hereditary spastic
paraplegia = bcS; sensory ataxia = bcT; gml-gangliosidosis, type ii = bCT; deafness, autosomal dominant 3a = bCt; acute
gastrointestinal hemorrhage = bct; d-2-hydroxyglutaric aciduria 1 = bcU; keratitis, ichthyosis, and deafness (kid) syndrome =
bCu; gml-gangliosidosis, type i = bCU; coumarin sensitivity = bcu; hyperkeratosis of the palms and soles and esophageal
papillomas = bCv; diaphragm disease = bcv; adult gml gangliosidosis = bCV; 2-hydroxyglutaricaciduria = bcV; d-2(oh) glutaric
aciduria = bcW; hid syndrome = bCw; amelogenesis imperfecta, type ib = bcw; spastic diplegia infantile type = bCW; otofaciocervical
syndrome = bcX; acroerythrokeratoderma = bCx; drug metabolism, poor, cyp2d6-related = bcx; nonpersistence = bCX; muscular
dystrophy-dystroglycanopathy (limb-girdle), type c, 9 = bcY; occlusive thrombus = bCY; wilms tumor 4 = bcy; deaf mutism =
bCy; recessive sensorineural hearing loss = bCz; gangliosidosis = bCZ; debrisoquine, poor metabolism of = bcz; alpha-dystroglycanopathyies =
bcZ; congenital heart disease = bD; rotavirus infections = BD; moyamoya disease = bd; refractive error = Bd; gangliosidosis
gm2 = bDa; angiokeratoma = bdA; cortical thymoma = bDA; lassa fever = bda; chromosome 15q11.2 deletion syndrome = bdB;
epithelial malignant thymom = bDB; delusion of persecution = bdb; lethal arthrogryposis with anterior horn cell disease =
bDb; necrobiosis lipoidica diabeticorum = bDc; delusional disorder = bdc; giant axonal neuropathy, autosomal dominant =
bdC; alacrima, achalasia, and mental retardation syndrome = bDC; synovial osteochondromatosis = bDd; mirror movements
1 = bdD; muscular dystrophy-dy stroglycanopathy (congenital with mental retardation), type b, 14 = bDD; conjunctival
disease = bdd; muscular dystrophy-dystroglycanopathy (limb-girdle), type c, 14 = bDE; holoprosencephaly 9 = bDe; pediatric
lymphoma = bde; developmental coordination disorder = bdE; specific reading disorder = bdF; mediastinal gray zone lymphoma =
bdf; pituitary anomalies = bDf; muscular dystrophy-dy stroglycanopathy (congenital with brain and eye anomalies), type
a, 14 = bDF; imperforate anus = bDg; van maldergem syndrome = bdG; hypomyelination with brainstem and spinal cord involvement
and leg spasticity = bdg; hypocalciuric hypercalcemia, familial, type 2 = bDG; hypocalcemia, autosomal dominant 2 = bDH;
spinal cord involvement = bdh; polydactyly, preaxial 4 = bDh; severe combined immunodeficiency with sensitivity to ionizing
radiation = bdH; drug resistant tuberculosis = bDI; reticuloendotheliosis, familial, with eosinophilia = bdI; leukoencephalyopathy
with brainstem and spinal cord involvement and lactate elevation = bdi; carpenter syndrome 1 = bDi; heavy metal toxicity =
bdj; severe combined immunodeficiency, partial = bdJ; postaxial polydactyly, type b = bDj; extensively drug-resistant
tuberculosis = bDJ; polydactyly of toes = bDk; familial ventricular tachycardia = bDK; corneal dystrophy, congenital
stromal = bdK; hantavirus infection = bdk; inflammatory bowel disease 12 = bDL; sex chromosome disorders of sex development =
bdl; ehlers-danlos syndrome progeroid type = bdL; carpenter syndrome = bDl; dopamine beta hydroxylase deficiency =
bdm; mammary paget's disease = bdM; nephronophthisis 7 = bDm; acidophil adenoma = bDM; dystonia 25 = bDN; atrophy of
kidney = bDn; autonomic nervous system diseases = bdn; neuronopathy, distal hereditary motor, type viib = bdN; segmental
dystonia = bDO; psychoticism = bdo; diabetes mellitus, neonatal, with congenital hypothyroidism = bDo; behavioral syndrome
associated with physiological disturbance and physical factors = bdO; foot-and-mouth disease = bdP; glomus vagale tumors =
bDp; epileptic encephalyopathy, early infantile, 17 = bDP; positive and negative symptoms = bdp; bullous pemphigoi =
bdQ; depressive psychosis = bdq; venous malformation = bDq; melanotic neurilemmoma = bDQ; central nervous system melanocytic
neoplasm = bDR; rift valley fever = bdR; severe major depression with psychotic features = bdr; congenital abnormality
of veins = bDr; hyperekplexia 2 = bDs; osseous heteroplasia, progressive = bDS; general symptom = bds; classical lissencephalyies
and subcortical band heterotopias = bdS; hypothyroidism, congenital, nongoitrous, 1 = bDT; woodhouse sakati syndrome =
bdt; lissencephaly and agenisi of corpus callosum = bdT; anemia, sideroblastic, pyridoxine-refractory, autosomal recessive =
bDt; leucinosis = bDu; pseudohypoparathyroidism type 1c = bDU; agyria = bdU; langer mesomelic dysplasia = bdu; atrial
standstill = bdv; toxic thyroid adenoma = bDV; glutamine deficiency, congenital = bDv; laminar heterotopia = bdV; toxic
multinodular goiter = bDW; epilepsy, cryptogenic = bdW; hhh syndrome = bDw; ambras syndrome = bdw; d-glyceric aciduria =
bDx; pentosuria = bdX; parvovirus infections = bdx; idiopathyic parathyroidism = bDX; d-glycericacidemia = bDy; chromosome
x pentasomy = bdy; calcinosis cutis = bDY; facial neoplasms = bdY; deficiency of aromatic-I-amino-acid decarboxylase =
bdZ; cutaneous mucormycosis = bdz; tay-sachs disease ab variant = bDz; brachymetacarpia = bDZ; myopia = Be; colon carcinoma =
bE; thoracic aortic aneurysm = be; invagination = BE; hunt's syndrome = bEA; adamantinoma = bEa; as if’ personality =
beA; aromatic amino acid decarboxylase deficiency = bea; juxtacortical osteosarcoma = bEb; spastic paraplegia 28, autosomal
recessive = beb; geniculate herpes zoster = bEB; epilepsy, partial, with variable foci = beB; aspartate aminotransferase,
serum level of, quantitative trait locus 1 = bEC; spastic paraplegia 54, autosomal recessive = bec; breast duct papilloma =
beC; ollier disease = bEc; chromophobe adenoma = beD; viral gastroenteritis due to rotavirus = bed; von willebrand
disease, platelet type = bED; night blindness, congenital stationary, autosomal dominant 3 = bEd; hepatosplenic t-cell
lymphoma = beE; bernard-soulier syndrome, type a2, autosomal dominant = bEE; pelizaeus-merzbacher disease = bee; charcot-marie-tooth
disease, dominant intermediate f = bEe; spondylometaepiphyseal dysplasia, short limb-hand type = bef; nonarteritic anterior
ischemiac optic neuropathy, susceptibility to = bEF; inclusion body myopathy autosomal recessive = bEf; cardiomyopathy,
dilated,1i= beF; vaso-occlusive crisis = bEG; granulomatous disorder = bEg; dwarfism, levi type = beg; muscular dystrophy,
limb-girdle, type 2r = beG; warsaw breakage syndrome = beh; blood vessel tumor = beH; glycine n-methyltransferase deficiency =
bEh; bleeding disorder, platelet-type, 11 = bEH; sporadic disorder = beI; rhizomelic chondrodysplasia punctata, type
2 = bEi; roberts syndrome = bei; bernard-soulier syndrome, type c = bEI; neoplasms, vascular tissue = beJ; ovarian embryonal
carcinoma = bEJ; spinal muscular atrophy with respiratory distress 1 = bej; glycosylphosphatidylinositol deficiency =
bEj; watermelon stomach disease = bek; hypertriglyceridemia, transient infantile = bEK; nephrotoxic serum nephritis =
bEk; deafness, autosomal dominant 5 = beK; brugada syndrome 2 = bEL; mucolipidosis ii alpha beta = bEl; deafness, autosomal
recessive 31 = beL; amyopathyic dermatomyositis = bel; adult-onset citrullinemia type 2 = bEM; gastric antral vascular
ectasis = bem; circling gait = beM; mucolipidosis iii gamma = bEm; hydrolethalus syndrome = ben; deafness, autosomal
recessive 59 = beN; cerebellar ataxia and hypogonadotropic hypogonadism = bEn; citrin deficiency = bEN; hypogonadotropic
hypogonadism 12 with or without anosmia = bEo; auditory neuropathy spectrum disorder = beO; cystinuria = beo; molybdenum
cofactor deficiency, complementation group c = bEO; t-lymphocyte deficiency = beP; flavivirus infections = bep; eunuchoidism,
familial hypogonadotropic = bEp; hemolytic anemia, nonspherocytic, due to glucose phosphate isomerase deficiency = bEP;
staring = bEq; sertoli cell-only syndrome, y-linked = beq; macrocephaly and epileptic encephalyopathy = beQ; opsoclonus
myoclonus = bEQ; glaucoma-related pigment dispersion syndrome = bER; hyperglycemia = beR; spermatocytoma = ber; fertile
eunuch syndrome = bEr; arterivirus infections = bes; nephrotic syndrome, type 7 = beS; acromegal = bES; vaccinia = bEs;
hemangioma of liver = bEt; mental retardation, x-linked 63 = beT; orofaciodigital syndrome i = bet; nystagmus 6, congenital,
x-linked = bET; night blindness, congenital stationary, type 1e = bEU; grade i astrocytoma = bEu; hypospadia = beU; infection
pyogenic = beu; induced hypothermia = bEV; deoxyguanosine kinase deficiency = beV; paraneoplasmtic encephalyomyelitis =
bev; urofacial syndrome = bEv; actinobacillus infections = bew; chudley-mccullough syndrome = bEW; globozoospermia =
bEw; lathosterolosis = beW; postoperative endophthalmiatis = bex; congenital hemidysplasia with ichthyosisform erythroderma
and limb defects = beX; gerodermaia osteodysplastica = bEx; keshan disease = bEX; tinea circinata = bey; loose anagen
hair syndrome = bEY; desmosterolosis = beY; tuberculosis, central nervous system = bEy; vaginal disease = bez; epilepsy,
progressive myoclonic 6 = bEz; glycoproteinosis = beZ; deafness, autosomal dominant 28 = bEZ; familial adenomatous polyposis =
bF; aortic aneurysm = bf; shigellosis = BF; deafness, autosomal dominant 20 = Bf; non-fluent aphasia = bFA; central
nervous system viral diseases = bFa; megaloblastic anemia due to dihydrofolate reductase deficiency = bfa; euthyroid
sick syndrome = bfA; mental retardation, fralZa type = bfB; dysgraphia = bFB; mass lesion = bFb; tetrahydrobiopterin
deficiency = bfb; subependymal glioma = bFc; schizophrenia 9 = bfC; central nervous system sensitization = bFC; deficiency
of dihydrofolate reductase = bfc; vascular dementi = bFd; brain dysfunction = bfD; 46,xy gonadal dysgenesis, partial,
with minifascicular neuropathy = bfd; deafness, autosomal recessive 25 = bFD; 46,xy gonadal dysgenesis, complete or partial,
dhh-related = bfe; severe recurrent major depression = bfE; abnormal extension = bFE; mental retardation, x-linked 94 =
bFe; rasmussen syndrome = bFf; short stature, auditory canal atresia, mandibular hypoplasia, skeletal abnormalities =
bFF; multiple personality disorder = bfF; pure gonadal dysgenesis 46,xy = bff; pure gonadal dysgenesis = bfg; dyskeratosis
congenita, x-linked = bfG; mental retardation, autosomal recessive 6 = bFg; hepatoma, novikoff = bFG; mixed gonadal dysgenesis =
bfh; hypophosphatemiac rickets, autosomal dominant = bfH; mental retardation, autosomal dominant 8 = bFh; meretoja
syndrome = bFH; hypophosphatemiac rickets, autosomal recessive, 1 = bfI; genee-wiedemann syndrome = bfi; epilepsy, focal,
with speech disorder and with or without mental retardation = bFi; lattice corneal dystrophy = bFI; inherited systemic
amyloidosis = bFJ; hyperostosis corticalis generalisata = bfJ; 2-aminoadipic 2-oxoadipic aciduria = bfj; rolandic epilepsy,
mental retardation, and speech dyspraxia, autosomal dominant = bFj; continuous spikes and waves during slow-wave sleep
syndrome = bFk; pyruvate dehydrogenase e2 deficiency = bfK; charcot-marie-tooth disease, axonal, type 2q = bfk; glutathione
synthetase deficiency of erythrocytes, hemolytic anemia due to = bFK; hereditary macular coloboma = bfl; glutathione
synthase deficiency with 5-oxoprolinuria = bFL; liver disease parenchymal = bfL; landau-kleffner syndrome = bFl; lymphogranuloma
venereum = bfm; ascorbic acid deficiency = bFM; mental retardation, autosomal dominant 6 = bFm; leukocyte adhesion deficiency,
type iii = bfM; deafness, autosomal dominant 64 = bfn; egg allergy = bFn; unspecified infectious and parasitic diseases =
bFN; lactic acidosis, congenital infantile, due to lad deficiency = bfN; deafness, autosomal dominant 1 = bfo; enhanced
s-cone syndrome = bFo; complete spermatogenic arrest = bFO; congenital immunodeficiency disease = bfO; mayer-rokitansky-kuster-hauser
syndrome = bfP; norwegian scabies = bFP; amaurosis congenita of leber, type 1 = bFp; premature ovarian failure 2a = bfp;
respiratory sounds = bFQ; mental retardation, x-linked 90 = bfQ; auditory neuropathy, autosomal dominant, 1 = bfq; oguchi
disease 2 = bFq; goiter, multinodular 1, with or without sertoli-leydig cell tumors = bfr; airway remodeling = bFR; retinal
cone dystrophy 1 = bfR; leber congenital amaurosis 12 = bFr; formaldehyde poisoning = bFS; rhabdomyosarcoma, embryonal,
2 = bfs; uniparental disomy, paternal, chromosome 14 = bfS; congenital stationary night blindnes = bFs; skeletal muscle
neoplasm = bfT; dicerl syndrome = bft; spinocerebellar ataxia, autosomal recessive 13 = bFt; drug-induced liver injury,
chronic = bFT; nonmedullary thyroid carcinoma = bfu; jarcho-levin syndrome = bfU; congenital cerebellar ataxia = bFu;
tumor virus infections = bFU; non-toxic multinodular goiter = bfv; delta-thalassemia = bfV; non-arteritic ischemiac optic
neuropathy = bFV; night blindness, congenital stationary, type 1b = bFv; left ventricular noncompaction = bfW; lipofuscinosis =
bFw; sex cord-gonadal stromal tumor = bfw; anemia in children = bFW; semantic impairment = bFx; hereditary persistence
of fetal haemoglobin [hpfh] = bfX; cone dystrophy 3 = bFX; endemic goiter = bfx; palinopsia = bFy; retinitis pigmentosa
48 = bFY; delta-beta thalassemia = bfY; kidney sarcoma = bfy; word finding difficulty = bFz; hand, foot and mouth disease =
bfZ; pleuropulmonary blastoma = bfz; moyamoya disease 6 with achalasia = bFZ; punctate palmoplantar keratoderma = bG;
baraitser-winter syndrome 2 = Bg; periodontal disease = bg; wiskott-aldrich syndrome = BG; spondylocostal dysostosis,
autosomal recessive = bga; perrault syndrome 2 = bGA; ovotesticular disorders of sex development = bgA; achalasi = bGa;
amelogenesis imperfecta, type iv = bgb; diarrhea 6 = bGb; progressive external ophthalmoplegia with mitochondrial dna
deletions, autosomal dominant, 6 = bgB; polymorphic reticulosis = bGB; taurodontism = bgc; clivus chordoma = bGC; ciliary
dyskinesia, primary, 13 = bgC; irritable bowel syndrome characterized by constipation = bGc; ige responsiveness, atopic =
bGD; retinal cone dystrophy 2 = bGd; auriculo-condylar syndrome = bgd; ciliary dyskinesia, primary, 10 = bgD; split-hand/foot
malformation with sensorineural hearing loss = bge; primary ciliary dyskinesia, 2 = bgE; chronic cervicitis = bGE; cone-rod
dystrophy 5 = bGe; cone-rod dystrophy 1 = bGf; nose neoplasms = bgf; neonatal purpura fulminans (homozygous protein c
deficiency) = bgF; mucormycosis = bGF; ciliary dyskinesia, primary, 18 = bgG; hereditary night blindness = bgg; histiocytosis
with joint contractures and sensorineural deafness = bGG; choroidal dystrophy central areolar = bGg; congenital stationary
night blindness = bgh; chromosome 7, trisomy 7q = bGh; heinz body anemias = bGH; heterotaxy, visceral, x-linked = bgH;
abnormal granulation tissue = bGi; limb-girdle muscular dystrophy, type 2c = bgi; ciliary dyskinesia, primary, 7 = bgI;
alpha-thalassemia mental retardation syndrome, deletion-type = bGI; anemia of pregnancy = bGJ; primary ciliary dyskinesia,
3 = bgJ; glycogen storage disease xv = bGj; hyperglycerolemia = bgj; hypoparathyroidism, x-linked = bgK; glycogen storage
disease 0, muscle = bGk; parkinson disease 2, autosomal recessive juvenile = bgk; severe alpha thalassemia = bGK; neuroacanthocytosis,
mcleod type = bgl; beta thalassemia minor = bGL; glycogen storage disease 0, liver = bGl; rhyns syndrome = bgL; hyaline
body = bgM; keratosis follicularis spinulosa decalvans, x-linked = bgm; hemoglobin bart's hydrops syndrome = bGM; epidermal
necrosis = bGm; maternally inherited leigh syndrome = bgN; gamma delta beta thalassemia = bGN; polyp benign = bGn; muscular
dystrophy, animal = bgn; normocytic hypochromic anemia = bGO; severe childhood autosomal recessive muscular dystrophy =
bgo; stunned myocardium = bgO; carotid stenosis, susceptibility to = bGo; congenital absence of adrenal gland = bgp;
delta thalassemia = bGP; hyperinsulinemiac hypoglycemia, familial, 4 = bGp; chronic pyelonephritis = bgP; hammer toe =
bGq; hemoglobin d trait = bGQ; dystrophyic cardiomyopathy = bgq; mitral valve disease = bgQ; merrf syndrome = bgR;
hemoglobin h constant spring = bGR; muscle fibrosis = bgr; carnitine-acylcarnitine translocase deficiency = bGr; cardiomyopathy
associated with another disorder = bgs; acute fatty liver of pregnancy = bGs; ciliary dyskinesia, primary, 9 = bgS; alpha-thalassemia,
deletion type = bGS; spinal muscular atrophy, distal, autosomal recessive, 5 = bgT; x-linked muscular dystrophy with
abnormal dystrophyin = bgt; reye-like syndrome = bGt; beta thalassemia, dominant inclusion body type = bGT; muscular
dystrophy, limb-girdle, type le = bgU; beta-sarcoglycanopathy = bgu; beta thalassemia major anemia = bGU; hemochromatosis,
type 3 = bGu; hemochromatosis, type 2a = bGv; retinoschisis = bgv; fetal hemoglobin quantitative trait locus 1 = bGV;
3-methylglutaconic aciduria, type v = bgV; hemoglobin e = bGW; hemochromatosis, type 2b = bGw; ceroid lipofuscinosis,
neuronal, 4b, autosomal dominant = bgW; fibrous meningioma = bgw; dominant thalassemia = bGX; bacterial cholangitis =
bGx; ciliary dyskinesia, primary, 16 = bgX; dimethylglycine dehydrogenase deficiency = bgx; charcot-marie-tooth disease,
x-linked, 1 = bgy; systemic lupus erythematosus 16 = bgY; laurin-sandrow syndrome = bGy; acute abdominal pain = bGY;
usher syndrome, type iiib = bGz; hypochromic anemia = bGZ; cardiogenic syncope = bgz; parental alcoholism = bgZ; nonsyndromeic
deafness = Bh; autoimmune hepatitis = bh; necrotizing fasciitis = bH; acute lung injury = BH; skin/hair/eye pigmentation,
variation in, 1 = bHA; hemoglobin c disease = bHa; hallucinogen persisting perception disorder = bha; ventricular fibrillation,
paroxysmal familial, 2 = bhA; color of iris = bHB; encephalyopathy, lethal, due to defective mitochondrial and peroxisomal
fission = bhb; penias carcinoma in situ = bHb; neuroleptic-induced tardive dyskinesia = bhB; gamma thalassemia = bHc;
spermatogenic failure 9 = bhC; spondylocostal dysostosis 4, autosomal recessive = bHC; charcot-marie-tooth disease, type
2b = bhc; spastic paraplegia type 4 = bhD; boomerang dysplasia = bhd; delta beta thalassemia = bHd; hypopituitarism and
septooptic ‘dysplasia’ = bHD; cyanosis, transient neonatal = bHe; growth hormone deficiency with pituitary anomalies =
bHE; dihydropyrimidinase deficiency = bhE; myopathy, centronuclear, autosomal dominant = bhe; central cyanosis = bHf;
panhypopituitaris = bHF; charcot-marie-tooth disease, dominant intermediate b = bhf; dihydropyrimidine dehydrogenase
deficiency = bhF; larsen syndrome = bhg; thymom = bhG; diffuse cutaneous mastocytosis = bHg; tay-sachs disease, juvenile =
bHG; epidural abscess = bHh; tay-sachs disease, variant b1 = bHH; lethal congenital contracture syndrome 5 = bhh; ciliary
dyskinesia, primary, 21 = bhH; inherited peripheral neuropathy = bhi; focal dermal hypoplasia = bHi; hexosaminidase alpha-subunit
deficiency (variant b) = bHI; tracheal stenosis = bhI; binge-eating disorder = bhJ; sick sinus syndrome 2, autosomal
dominant = bHj; hereditary sensory and autonomic neuropathy type 1e = bhj; infantile sandhoff disease = bHJ; cerebellar
ataxia, deafness, and narcolepsy, autosomal dominant = bhk; brugada syndrome 8 = bHk; opium abuse = bhK; sandhoff disease,
adult type = bHK; emotional stress = bhL; tachy-brady syndrome = bHl; cerebellar ataxia, deafness, and narcolepsy = bhl;
sandhoff disease, infantile type = bHL; childhood aggression = bhM; latent autoimmune diabetes mellitus in adult = bhm;
sick sinus syndrome = bHm; sandhoff disease, juvenile type = bHM; victimizing others = bhN; juvenile sandhoff disease =
bHN; adams-oliver syndrome 2 = bhn; narcolepsy 1 = bHn; cortical blindness = bho; hemochromatosis, type 4 = bHO; hypersomnolence =
bHo; acute schizophrenia = bhO; air cyst = bhp; drug use disorders = bhP; hfe gene = bHP; narcolepsy without cataplexy =
bHp; job's syndrome = bhq; porphyria, south african type = bHQ; hypersomnia, post-traumatic = bHq; cluster a personality
disorder = bhQ; microvascular complications of diabetes, susceptibility to, 7 = bHR; keratoconu = bhr; chondrodysplasia
with platyspondyly, distinctive brachydactyly, hydrocephaly, and microphthalmia = bHr; opioid use = bhR; opium addiction =
bhS; pseudomyopathyic myasthenia = bhs; cornealia de lange syndrome 5 = bHs; hepatitis, viral, human = bHS; schizoid
personality disorder = bhT; proximal myopathy = bht; chronic fatigue = bHT; wilson-turner x-linked mental retardation
syndrome = bHt; pathyological gambling = bhU; congenital disorder of glycosylation, type 1m = bhu; flaviviridae infections =
bHU; acute intestinal obstruction = bHu; gambling = bhV; primary hypertriglyceridemia = bHV; congenital disorder of
glycosylation type 1j = bhv; megalencephalyic leukoencephalyopathy with subcortical cysts 2a = bHv; metal metabolism
disorder = bHW; tremor hereditary essential, 1 = bhW; megalencephalyic leukoencephalyopathy with subcortical cysts 2b,
remitting, with or without mental retardation = bHw; myasthenic syndrome, congenital, with tubular aggregates 2 = bhw;
macrocephaly, benign familial = bHx; congenital disorder of glycosylation, type 1u = bhx; obsessive-compulsive personality
disorder = bhX; selective igg deficiency disease = bHX; rapadilino syndrome = bHY; oculocutaneous albinism type 2 = bHy;
congenital disorder of glycosylation, type 1o = bhy; novelty seeking personality trait = bhY; mental retardation, autosomal
recessive 38 = bHz; premature ovarian failure 9 = bHZ; adenomatoid tumor = bhz; hyperkinetic conduct disorder = bhZ;
delayed sleep phase syndrome, susceptibility to = bI; immune reconstitution inflammatory syndrome = Bi; in situ carcinoma =
BI; paget's disease of bone = bi; hair morphology 2 = biA; hemolytic anemia due to hexokinase deficiency = bIA; finding
relating to sexuality and sexual activity = bia; ochronotic arthropathy = bIa; eye infections, fungal = bIB; moderate
obesity = bib; dentin dysplasia, type ii = biB; ochronotic arthritis = bIb; dentinogenesis imperfecta, shields type 3 =
biC; alveolar echinococcosis = bIC; female sexual dysfunction = bic; ochronosis = bIc; dependent personality disorder =
bid; paraneoplasmtic syndromes, nervous system = bID; alkaptonuri = bId; deafness, autosomal dominant 39, with dentinogenesis
imperfecta 1 = bil; sexually transmitted diseases, viral = bIE; diarrhea 3, secretory sodium, congenital = bIe; iga pemphigus =
bie; hereditary opalescent dentin (disorder) = biE; subcorneal pustular dermatosis = bif; arthrofibrosis = bIF; deafness,
autosomal recessive 39 = bIf; epidermaolysis bullosa simplex, autosomal recessive = biF; arrhythmogenic right ventricular
dysplasia, familial, 11 = big; neuropathy, hereditary sensory and autonomic, type vi = biG; ulcer of artery = bIg; plasmodium
falciparum infection = bIG; hypotrichosis and recurrent skin vesicles = bih; bites and stings = bIH; epidermaolysis bullosa
simplex, autosomal recessive 2 = biH; cutaneous neurofibroma = bIh; drug reaction with eosinophilia and systemic symptoms =
bII; renal hypodysplasia, nonsyndromeic, 1 = biI; cardiomyopathy, right ventricular = bii; fibrosis of urinary bladder =
bIi; ehlers-danlos syndrome, musculocontractural type 2 = bij; noncompaction of left ventricular myocardium, familial
isolated, autosomal dominant 1 = biJ; chest--diseases = bIj; ocular myasthenia gravis = bIJ; lumbar meningomyelocele =
bIK; keratosis palmoplantaris striata 1 = bik; nephronophthisis - medullary cystic disease = bIk; hermansky-pudlak
syndrome 7 = biK; moderate chronic obstructive pulmonary disease = biL; erythroderma, congenital, with palmoplantar keratoderma,
hypotrichosis, and hyper-ige = bil; involutional paraphrenia = bIL; familial renal papillary carcinoma = bIl; crimean-congo
hemorrhagic fever = bIm; thyroid dyshormonogenesis 6 = biM; ritter's disease = bim; birdshot chorioretinitis = bIM; conjunctivitis =
bIN; chromosome lp36 deletion syndrome = bIn; hormone deficiency = biN; arrhythmogenic right ventricular dysplasia,
familial, 10 = bin; subclinical hypothyroidism = biO; cardiomyopathy, dilated, ibb = bio; pars planitis = bIO; beta-hydroxyisobutyryl
coa deacylase deficiency = bIo; male germ cell tumor = bIp; transient hypothyroidism = biP; skeletal tuberculosis = bIP;
pemphigus vulgaris, familial = bip; disorder of mucous membrane = biq; thyroid dyshormonogenesis 5 = biQ; nut allergic
reaction = bIQ; myocardial hypoxia = bIq; hypotrichosis, localized, autosomal recessive 1 = bir; pityriasis rosea = bIR;
dyshormonogenic goiter = biR; myeloma, endothelial = bIr; epidermal growth factor receptor negative non-small cell lung
cancer = bIs; systemic vasculitis = bIS; klatskin's tumor = bis; monilethrix = bis; acyanotic congenital heart disease =
bIt; hypogonadotropic hypogonadism 19 with or without anosmia = biT; skin fragility-woolly hair syndrome = bit; abacavir
hypersensitivity, susceptibility to = bIT; malocclusion, angle class iii = biU; back pain mechanical = bIU; spindle cell
hemangioma = bIu; arrhythmogenic right ventricular dysplasia, familial, 8 = biu; cysticercosis = biV; naxos disease =
biv; non-congenital cyst of kidne = bIv; corneal degeneration = bIV; tenosynovitis = bIW; ectodermal dysplasia/skin fragility
syndrome = biw; neurobehavioral problems = bIw; potocki-lupski syndrome = biW; renal glycosuria = bIX; spinal muscular
atrophy, childhood, proximal, autosomal dominant = bix; cardiomyopathy dilated with woolly hair and keratoderma = bix;
typhoid feve = bIx; hemolytic anemia, nonspherocytic, due to hexokinase deficiency = bIy; epidermaolysis bullosa, lethal
acantholytic = biy; charcot-marie-tooth disease, axonal, type 20 = biY; psoriasis susceptibility 1 = bIY; mental retardation,
autosomal dominant 13 = biZ; neuropathy, hereditary motor and sensory, russe type = bIz; guttate psoriasis = bIZ; keratosis
palmoplantaris striata ii = biz; cowden disease = bj; body mass = bJ; childhood leukemia = Bj; granulosa cell tumor =
BJ; asphyxiating thoracic dystrophy 3 = bja; conjunctival scar = bJa; hypohidrotic ectodermal dysplasia = bjA; macrophagic
myofasciitis = bJA; polyglandular failure = bJB; tooth agenesi = bjB; mainzer-saldino disease = bjb; fissured tongue =
bJb; ectodermal dysplasia 3, anhidrotic = bjC; lupus cutaneous = bJC; scalp dermatosis = bJc; short rib-polydactyly
syndrome, verma-naumoff type = bjc; ectodermal dysplasia 11a, hypohidrotic/hair/tooth type, autosomal dominant = bjD;
leg dermatosis = bJd; undifferentiated inflammatory arthritis = bJD; vacterl association = bjd; sclerosisng lymphocytic
lobulitis = bJE; dmac = bJe; mental retardation, autosomal dominant 7 = bje; ectodermal dysplasia flb, hypohidrotic/hair/tooth
type, autosomal recessive = bjE; physical symptom = bjf; question mark ears, isolated = bjF; opticospinal multiple sclerosis =
bJf; subchondral cyst = bJF; color blindnes = bjg; ige-mediated allergic asthma = bJG; auriculocondylar syndrome 3 =
bjG; tungsten-carbide disease = bJg; renal involvement in scleroderma = bJH; small-vessel disease = bjH; arthritis
of hip = bJh; myopathy, distal, with anterior tibial onset = bjh; welander distal myopathy = bji; borderline leprosy =
bJI; gluten sensitivity = bJi; diabetic heart disease = bjI; strongyloidiasis = bJJ; uterovaginal prolapse = bjJ; fibrocalculous
pancreatic diabetes = bJj; ciliary dyskinesia, primary, 25 = bjj; fibrosis of bile duct = bjk; elephantiasis = bjK; latex
hypersensitivity = bJk; synovitiss = bJK; hepatosplenic schistosomiasis = bJl; benign peritoneal mesothelioma = bjl;
acute chest syndrome = bjL; iritis = bJL; folliculitis = bJM; macrocytosis, familial = bjm; allergic fungal sinusitis =
bJm; waardenburg syndrome, type 4b = bjM; lymph node disease = bjn; diffuse infiltrative lymphocytosis syndrome = bJN;
waardenburg syndrome, type 4 = bjN; celiac disease in children = bJn; hirschsprung disease, susceptibility to, 4 = bjO;
microscopic colitis = bJO; sequoiosis = bJo; paranoid traits = bjo; allergy to fruit = bJp; hepatic cancer metastatic =
bjP; echovirus infections = bJP; combined oxidative phosphorylation deficiency 12 = bjp; chondrodysplasia punctata
2, x-linked dominant = bjq; acute disseminated encephalyomyelitis = bJq; impetigo = bjQ; sarcoid arthropathy = bJQ; whipple
disease = bJr; hirschsprung disease, susceptibility to, 2 = bjR; major histocompatibility complex class ii deficiency =
bJR; arthrogryposis, distal, type 5d = bjr; abcd syndrome = bjS; camptodactyly acquired = bjs; subacute thyroiditis =
bJS; multiple sclerosis, susceptibility to = bJs; insulin autoimmune syndrome = bJt; epiglottitis = bJT; camptodactyly
congenital = bjt; digestive system abnormalities = bjT; congenital intestinal aganglionosis = bjU; intravenous bisphosphonates =
bJU; echinococcosis, hepatic = bJu; tooth agenisi, selective, x-linked, 1 = bju; pigeon-fanciers' disease = bJv; ectodermal
dysplasia (anhidrotic) = bjv; neuronal intestinal dysplasia = bjV; autoimmune vasculitis = bJV; hypohidrosis = bjw; disorder
of the genitourinary system = bJw; wasting = bjW; erysipelothrix infections = bJW; herpes simplex type 1 infection =
bJX; epidemic nephropathy = bJx; anhidrosis = bjx; chronic idiopathyic urticaria = bjX; lung sarcoma = bJY; actinic prurigo =
bJy; malattia leventinese = bjY; anodontia = bjy; porphyria, acute intermittent, nonerythroid variant = bJZ; craniofrontonasal
syndrome = bjz; dominant drusen = bjZ; ankle arthritis = bJz; visceral myopathy, familial = Bk; bipolar disorder = bK;
axonal neuropathy = bk; thalamic neoplasm = BK; pituitary eosinophilic adenoma = bka; porphobilinogen deaminase deficiency =
bKa; amyotrophyic lateral sclerosis 20 = bKA; alkaline phosphatase, plasma level of, quantitative trait locus 1 = bkA;
pulmonary lymphangiomyomatosis = bKb; inclusion body myopathy with early-onset paget disease with or without frontotemporal
dementia 2 = bKB; cutis laxa, autosomal recessive, type 1b = bkb; pancreatic cancer stage = bkB; cutis laxa, recessive =
bkc; multiple basal cell papillomata = bkC; athabaskan brainstem dysgenesis = bKC; lipoma of colon = bKc; vagina leiomyoma =
bKd; epilepsy with generalized tonic-clonic seizure = bkd; larynx squamous papilloma = bkD; bosley-salih-alorainy syndrome =
bKD; periosteal disorder = bke; benign mesenchymoma = bKe; thrombocytopenia-absent radius syndrome = bKE; dermaoid
cyst = bkE; mixed cell type cancer = bkF; venezuelan equine encephalyitis = bkf; myofibroma = bKf; radioulnar synostosis
with amegakaryocytic thrombocytopenia = bKF; statins, attenuated cholesterol lowering by = bKg; growth and mental retardation,
mandibulofacial dysostosis, microcephaly, and cleft palate = bkg; adenosarcoma = bkG; pelvic prolapse = bKG; heme oxygenase
1 deficiency = bKh; acute interstitial pneumonia = bkH; hand foot uterus syndrome = bKH; esophageal atresia = bkh; invasive
malignant thymoma = bkI; axenfeld-rieger syndrome, type 1 = bki; preaxial deficiency, postaxial polydactyly and hypospadias =
bKI; infection by leishmania infantum = bKi; microtia, hearing impairment, and cleft palate = bKJ; hypomagnesemia 4,
renal = bkj; breast metaplastic carcinoma = bkJ; pancreatitis, graft = bKj; pineal parenchymal tumor of intermediate
differentiation = bkK; spastic paraplegia 20, autosomal recessive = bkk; facial paresis, hereditary congenital, 3 = bKK;
pleurisy = bKk; facial paresis, hereditary, congenital = bKL; mixed glioma = bkL; oculoauricular syndrome = bKl; fast
acetylator due to n-acetyltransferase enzyme variant = bkl; mature teratoma = bkM; maturity-onset diabetes of the young,
type 3 = bKm; mesenchymal glioblastoma = bkm; constant squint = bKM; myxopapillary ependymom = bKN; laron syndrome type
2 = bKn; peripheral nerve sheath neoplasm = bkn; cervical adenosquamous carcinoma = bkN; lymphoepithelioma-like carcinoma =
bkO; trophyoblastic neoplasm = bko; diabetes mellitus, insulin-dependent, 20 = bKo; ectodermal dysplasia, pure hair-nail
type = bKO; uterine carcinosarcoma = bkP; papillary transitional carcinoma = bkp; lower extremity deformities, congenital =
bKP; hepatic adenomas, familial = bKp; kidney angiomyolipoma = bkq; syndactyly, type v = bKQ; esophageal basaloid squamous
cell carcinoma = bkQ; insulin resistance, susceptibility to = bKq; brachydactyly, type d = bKR; molluscum contagiosum =
bkr; erythrocytosis, familial, 3 = bkR; low renal threshold for glucose = bKr; brachydactyly-syndactyly syndrome =
bKS; juvenile gout = bKs; pneumonia, staphylococcal = bks; maladaptive behavior associated with physical illness = bkS;
synpolydactyly with foot anomalies = bKT; amino acid transport disorder = bkT; otorhinolaryngologic neoplasms = bkt;
medullary cystic kidney disease 1 = bKt; primary polycythemia = bkU; prostate cancer, hereditary, 11 = bKu; recurrent
lung cancer = bku; simple syndactyly of toes, first web space = bKU; brachymesophalangy 2 and 5 = bKV; hyperuricemiac
nephropathy = bKv; retroperitoneal sarcoma = bkV; bronchial dysplasia = bkv; medullary cystic kidney = bKw; anhaptoglobinemia =
bKW; charcot-marie-tooth disease, type 4e = bkW; familial ependymoma = bkw; cerebral metastases = bkx; foodborne diseases =
bKX; charcot-marie-tooth disease, type 1d = bkX; maturity-onset diabetes of the young, type 1 = bKx; dysplastic oral
leukoplakia = bky; dejerine-sottas disease = bkY; senile systemic amyloidosis = bKY; detoni fanconi syndrome = bKy; inclusion
body myopathy with early-onset paget disease with or without frontotemporal dementia 3 = bkz; asthmatic bronchitis =
bkz; carotid disease = bKZ; myelinopathy = bkZ; myositis ossificans = BL; muscular atrophy = bl; megaduodenum and/or
megacystis = Bl; type 2 diabetes mellitus = bL; scurvy = bLa; prostate cancer, hereditary, 12 = bla; fetal adenoma =
bLA; septicemia due to escherichia coli [e. coli] = blA; thrombophilia due to histidine-rich glycoprotein deficiency =
bLB; enterococcal infection = blb; air trapping = blB; tyrosinemia type iii = bLb; thrombophilia due to elevated histidine-rich
glycoprotein = bLC; hawkinsinuria = bLc; biphasic synovial sarcoma = blC; hypothalamic syndrome = blc; bell's palsy =
bLD; staggering gait = bld; pancreatic islet cell tumors = blD; digital clubbing, isolated congenital = bLd; cranioosteoarthropathy =
bLe; hypogonadotropic hypogonadism 15 with or without anosmia = bLE; deafness, autosomal recessive 88 = blE; polymicrogyria
with optic nerve hypoplasia = ble; buschke-ollendorff syndrome = blF; fanconi renotubular syndrome 3 = blf; unilateral
undescended testis = bLf; cortisone reductase deficiency = bLF; cortisone reductase deficiency 2 = bLG; glucagonoma =
blg; cutis laxa, autosomal dominant 1 = blG; basal cell carcinoma, multiple = bLg; hypothalamic obesity = bLH; kleefstra
syndrome = blh; cutis laxa, autosomal dominant = blH; deficiency of transferase = bLh; apparent mineralocorticoid excess
syndrome = bLI; hemangiomatosis, familial pulmonary capillary = bli; self-injurious behavior = bLi; fenestration = blI;
elastosis perforans serpiginosa = blJ; acute urate nephropathy = bLj; hydroxysteroid (11-beta) dehydrogenase 2 = bLJ;
amino acid deficiency = blj; hyporeninemiac hypoaldosteronism = bLK; hps3 gene = bLk; vanishing white matter leukodystrophy
with ovarian failure = blk; proximal aortic dissection = blK; hermansky-pudlak syndrome 4 = bLl; vanishing white matter
disease = bll; hypertensive end stage renal disease = bLL; cutis laxa, acquired type = blL; constrictive bronchiolitis =
blM; mental retardation, x-linked, syndromeic 10 = bLM; hps5 gene = bLm; hereditary central nervous system demyelinating
diseases = blm; mental retardation, x-linked 17 = bLN; congenital supravalvular aortic stenosis = blN; glycogen storage
disease iiib = bln; hps6 gene = bLn; alloimmune thrombocytopenia = bLo; deficiency of enoyl-coa hydratase = bLO; osteopoikilosis =
blO; glycogen synthase deficiency = blo; pulmonary valve stenosis = blP; breast lymphoma = bLp; beta-ketothiolase deficiency =
bLP; hyperkalemic periodic paralysis = blp; bile acid synthesis defect, congenital, 1 = bLQ; richieri costa pereira
syndrome = blq; cone-rod dystrophy 7 = blQ; diffuse alveolar damage = bLq; stargardt disease 3 = blR; autism, susceptibility
to, 19 = blr; hajdu-cheney syndrome = bLr; cataract, zonular = bLR; ichthyosis, spastic quadriplegia, and mental retardation =
blS; parkinson disease 18 = bls; compartment syndrome = bLs; congenital lamellar cataract = bLS; chronic hand eczema =
blT; transposition = bLT; alopecia universalis congenita = bLt; cardiovirus infections = blt; cat-scratch disease =
blU; asthma, occupational = blu; marie unna hereditary hypotrichosis 1 = bLu; primary cortisol resistance = bLU; limb-girdle
muscular dystrophy, type 1b = blV; prostate cancer, hereditary, 2 = blv; endocrine disease of the skin = bLV; alopeci =
bLv; bowen-conradi syndrome = blW; protozoan diseases = bLW; combined oxidative phosphorylation deficiency 17 = blw;
myopathy, congenital, with excess of muscle spindles = bLw; familial schizencephaly = blX; plasminogen deficiency, type
1 = blx; sighing respiration = bLX; endemic diseases = bLx; amelogenesis imperfecta, type 1c = blY; parasitic protozoa
infectious disease = bLY; neutropenia, severe congenital, autosomal dominant 1 = bly; phacomatosis pigmentokeratotica =
bLy; neonatal meningitis = bLZ; open bite = blZ; plantar wart = bLz; ligneous conjunctivitis = blz; asthma = bM; abdominal
aortic aneurysm = bm; lateral meningocele syndrome = Bm; atrioventricular septal defect = BM; dental enamel hypoplasia =
bma; hemoglobin c trait = bmA; leukodystrophy, hypomyelinating, 4 = bMa; peripheral neuropathyic pain = bMA; infectious
gastroenteritis = bMb; saccular aneurysm = bmb; vici syndrome = bmB; major affective disorder = bMB; segmental vitisligo =
bmc; alcoholic psychosis = bMC; gestosis = bmC; high altitude illness = bMc; corneal dystrophy, fuchs' endothelial,
2 = bmD; sarcoid uveitis = bMd; sexual dysfunction, physiological = bMD; lymphocytic hypopituitarism = bmd; resectable
hepatocellular carcinoma = bMe; glycogen storage disease xiii = bme; perseveration = bME; myotonic dystrophy type 2 =
bmE; drug-induced depressive state = bMF; adducted thumbs syndrome = bmf; charcot-marie-tooth disease, type 2f = bMf;
congenital absence of liver = bmF; ureterocele = bmG; hallucinosis = bMG; hypophosphatemiac rickets, autosomal recessive,
2 = bmg; neuronopathy, distal hereditary motor, type iib = bMg; upper motor neuron sign = bMh; cole disease = bmh; bulla
of lung = bmH; hyperdistention = bMH; spondyloarthropathy, susceptibility to, 1 = bmi; system disorder of the nervous
system = bMI; microvascular complications of diabetes, susceptibility to, 2 = bmI; neuronopathy, distal hereditary motor,
type iic = bMi; macular degeneration, age-related, 7 = bMJ; bile duct disease = bmj; charcot-marie-tooth disease, type
2i = bMj; chronic anemia = bmJ; cadmium poisoning = bmk; confluent drusen = bMK; neuropathy, distal hereditary motor,
type iia = bMk; heterozygous hemoglobinopathy = bmK; spastic paraplegia 13, autosomal dominant = bMl; anemia in malignant
neoplasmtic disease = bmL; spastic paraplegia 64, autosomal recessive = bml; parkinson disease 13, autosomal dominant,
susceptibility to = bML; inguinal lymphadenopathy = bmM; juvenile onset huntington's disease = bMM; head injuries, penetrating =
bmm; spastic paralysis = bMm; anemia of chronic renal failure = bmN; fourth disease = bMN; adams-oliver syndrome 4 =
bmn; diffuse lepromatous leprosy = bMn; mental retardation, x-linked, syndromeic, turner type = bMO; anemia of renal
disease = bmO; hypoxia, brain = bmo; inflammatory acne = bMo; anemia of prematurity = bmP; rubinstein-taybi syndrome
2 = bmp; nocardiosis = bMp; hyaluronidase deficiency = bMP; ossifying fibromyxoid tumor = bmq; ciliary dyskinesia, primary,
5 = bMQ; dyssegmental dysplasia = bMq; periventricular leukomalacia = bmQ; erythrocytosis, familial, 4 = bmr; phosphohydroxylysinuria =
bMR; cerebellar angioblastoma = bmR; periodic fever, menstrual cycle-dependent = bMr; hydroxylysinuria = bMS; diabetic
macular edema = bmS; respiratory distress syndrome in premature infants = bms; depression anxiety disorder = bMs; somatostatinoma =
bmt; hydrolethalus syndrome 1 = bMT; affective symptoms = bMt; myeloperoxidase deficiency = bmT; offensive aggression =
bMu; pancreatic somatostatinoma = bmu; presentey anomaly = bmU; multiple mitochondrial dysfunctions syndrome 3 = bMU;
aura = bMv; corneal dystrophy, posterior amorphous = bmV; mental retardation, autosomal dominant 11 = bmv; central neuroblastoma =
bMV; spherocytosis, type 5 = bmw; juvenile psoriatic arthritis = bmW; sleep arousal disorders = bMw; malaria, cerebral,
susceptibility to (finding) = bMW; diurnal enuresis = bMx; diarrhea 5, with tufting enteropathy, congenital = bmx; stasis
dermatitis = bMX; glioma susceptibility 1 = bmX; colorectal cancer, hereditary nonpolyposis, type 8 = bmy; prodromal
states = bMy; disorder characterized by fever = bMY; congenital duodenal obstruction due to malrotation of intestine =
bmY; lynch syndrome i (site-specific colonic cancer) = bmz; keratopathy = bMZ; sleep bruxism = bMz; radicular pain =
bmZ; neoplasms, second primary = BN; disorder of smooth muscle = Bn; achalasia = bn; autistic disorder = bN; landsteiner-wiener
phenotype = bNa; trichothiodystrophy, photosensitive = bnA; somatoform disorder = bNA; periodic alternating nystagmus =
bna; epidermal growth factor receptor positive non-small cell lung cancer = bnb; intestinal perforation = bNB; brittle
hair = bnB; endocrine-cerebroosteodysplasia = bNb; henipavirus infections = bNC; epidermaolytic hyperkeratosis = bnC;
colon (non-specific) lesion = bnc; immunodeficiency, common variable, 1 = bNc; xeroderma pigmentosum b/cockayne syndrome =
bnD; cerebellar degeneration, subacute = bnd; chronic eczema = bNd; skin papilloma = bND; neurocytoma = bNe; fanconi
anemia, complementation group q = bnE; hepatitis b virus, susceptibility to = bNE; hormone receptor positive tumor =
bne; chromosome 8, trisomy 8p = bNf; paraneoplasmtic cerebellar degeneration, anti-yo-associated = bnf; sendai virus
infection = bNF; radiation sickness = bnF; xeroderma pigmentosum, type g/cockayne syndrome = bnG; rebound nystagmus =
bng; entamoebiasis = bNG; chondroblastic osteosarcoma = bNg; salmonella infections, animal = bNH; uv-sensitive syndrome
1 = bnH; uterine corpus cancer = bnh; retinitis pigmentosa 46 = bNh; pneumoconiosis = bni; aplastic anemia, susceptibility
to (finding) = bNI; bone marrow failure syndrome 2 = bnI; paroxysmal extreme pain disorder = bNi; uv-sensitive syndrome
2 = bnJ; dermaopathy = bNJ; alpha-l-iduronidase deficiency = bNj; ovary epithelial cancer = bnj; microcephaly, epilepsy,
and diabetes syndrome = bNk; enterobacteriaceae infections = bNK; empty sella syndrome = bnk; craniosynostosis 4 = bnK;
gastroesophageal junction adenocarcinoma = bnl; microcephaly with simplified gyral pattern = bNl; picornaviridae infections =
bNL; synostotic posterior plagiocephaly = bnL; complex craniosynostosis = bnM; diabetes mellitus, insulin-dependent,
19 = bNm; apocrine adenosis of breast = bnm; heymann nephritis = bNM; breast apocrine carcinoma = bnn; aplasia of muscle =
bNn; primary bacterial peritonitis = bNN; friend leukemia = bnN; osteogenesis imperfecta, type v = bNo; typhlocolitis =
bNO; gastric tubular adenocarcinoma = bno; mild visual impairment = bnO; fundus albipunctatus = bnP; osteogenesis imperfecta,
type vi = bNp; breast intraductal proliferative lesion = bnp; fatal infectious mononucleosis = bNP; lethal congenital
contracture syndrome 1 = bnq; blue color blindness = bnQ; myd88 deficiency = bNq; bronchopulmonary aspergillosis = bNQ;
specific viral infections = bNr; ectomesenchymoma = bnR; lethal congenital contracture syndrome 2 = bnr; fungal keratitis =
bNR; prostate small cell carcinoma = bnS; spirochaetales infections = bNS; paresthesia, distal = bNs; secondary malignant
neoplasm of the omentum = bns; spastic paraplegia 18, autosomal recessive = bnT; choroid plexus papilloma = bnt; glomerulonephritis
proliferative chronic = bNT; myocarditis, chronic = bNt; tuberculous pleural effusion = bNu; amyotrophyic lateral sclerosis
19 = bnu; sle glomerulonephritis syndrome, who class iv = bNU; antigen in scianna blood group system = bnU; disseminated
due to other mycobacteria = bNv; atopic cataract = bNV; radiculopathy = bnv; radin blood group antigen = bnV; xfe progeroid
syndrome = bnw; periventricular nodular heterotopia 6 = bnW; avian influenza = bNw; toxoplasmosis chorioretinitis = bNW;
melanoma recurrent = bNX; cerebrooculofacioskeletal syndrome 4 = bnx; vipoma = bNx; congenital heart bloc = bnX; erythromelalgia =
bNy; gastrointestinal toxicity = bny; pseudoainhum = bnY; drug-induced erythema multiforme = bNY; hepatitis d, chronic =
bNz; focal infection = bnZ; cerebrooculofacioskeletal syndrome 2 = bnz; autologous graft versus host disease = bNZ;
lynch syndrome = BO; congenital dilatation of bladder = Bo; endogenous depression = bO; hypoglycemia = bo; vacterl association
with hydrocephalyus = bOA; myocarditis, active = bOa; argentinian hemorrhagic fever = boa; premenstrual symptom = boA;
aseptic peritonitis = bob; hernia sac = boB; cranioectodermal dysplasia 3 = bOB; chlamydophila pneumoniae infections =
bOb; asphyxiating thoracic dystrophy 2 = bOC; eosinophilic pustular folliculitis = bOc; murray valley encephalyitis =
boc; deafness, autosomal recessive 35 = boC; corpus callosum, agenisi of, with mental retardation, ocular coloboma,
and micrognathia = bOD; western equine encephalyitis = bod; lattice corneal dystrophy type 1 = boD; panarteritis = bOd;
glossitis = boe; wrist injuries = bOE; male sexual dysfunction = boE; mycobacterium leprae infection = bOe; osteodysplasia =
bOF; zoonotic form of cutaneous leishmaniasis = bOf; colorado tick feve = bof; bacteroides infections = boF; overproduction
of growth hormone = bOG; disseminated coccidioidomycosis = bOg; mesoblastic nephroma, cellular = boG; bilateral retinoblastoma =
bog; secondary generalized epilepsy = bOh; cystic hypersecretory carcinoma = boH; axial myopia = bOH; deafness, autosomal
recessive 36, with or without vestibular involvement = boh; weyers acrofacial dysostosis = boI; disseminated bacillus
calmette-guerin infection = bOi; deafness, autosomal recessive 36, without vestibular involvement = boi; catabolic state =
bOI; smooth muscle cancer = bOJ; corneal ulcer = bOj; ellis-van creveld syndrome = boJ; alzheimer disease 12 = boj;
trichuriasis = bOk; suicidal behavior = bok; hidradenoma = boK; ring chromosome 15 syndrome = bOK; campylobacteriosis =
bOl; vulvar vestibulitis = bol; muscle cancer = bOL; cauda equina neoplasm = boL; in vitro fertilization = bom; sarcomatosis =
boM; diabetes mellitus, insulin-dependent, 2 = bOM; cystic echinococcosis = bOm; lumbar spondylosis = bon; rapidly
involuting congenital hemangioma = bON; chilblain lupus = boN; endophthalmitis = bOn; non-involuting congenital hemangioma =
bOO; mucopolysaccharidosis v = boO; chronic rhinitis = bOo; prolapse of female genital organs = boo; mycobacterium
infections, nontuberculous = bOp; postpartum psychosis = bop; experimental organism islet cell adenoma neoplasm = bOP;
dermatitis herpetiformi = boP; atypical carcinoid tumor = boq; mycobacterial disease, mendelian susceptibility to = bOq;
pontocerebellar hypoplasia type 1 = boQ; mesoblastic nephroma = bOQ; meninges hemangiopericytoma = bOR; pontocerebellar
hypoplasia, type 1b = boR; interferon gamma receptor deficiency = bOr; vulvar intraepithelial neoplasmia, usual type =
bor; polyhydramnios = bOS; disseminated atypical mycobacterial infection = bOs; male genital abnormalities = bos; infection
by leishmania mexicana = boS; omphalocele = bOT; epidermaolysis bullosa, nonspecific, autosomal recessive = bol; helicobacter
pylori infection, susceptibility to = bOt; desquamative gingivitis = bot; infection in children = bOu; epidermaolysis
bullosa simple = boU; papillary urothelial carcinoma = bou; diabetes mellitus, insulin-dependent, 8 = bOU; microglial
nodules = bOV; exostoses, multiple, type ii = boV; ovary transitional cell carcinoma = bov; primary tuberculosis = bOv;
leprosy in children = bOw; metachondromatosis = boW; breast adenoid cystic carcinoma = bow; semantic dementia = bOW;
locked-in syndrome = bOX; cranioectodermal dysplasia = bOx; sclerosisng hemangioma = box; renal failure, progressive,
with hypertension = boX; fragile x chromosome = boY; immature teratoma of ovary = boy; chronic liver failure = bOY; progressive
diaphyseal dysplasia = bOy; posttransfusion viral hepatitis = boZ; perivascular epithelioid cell tumor = bOZ; testicular
hydrocele = boz; short-rib thoracic dysplasia 10 with or without polydactyly = bOz; barth syndrome = bp; retinoblastoma =
bP; weissenbacher-zweymAAller syndrome = BP; spina bifida cystica = Bp; adrenal neuroblastoma = bPa; dysplasia epiphysealis
hemimelica = bpa; chronic malaria = bPA; hearing loss, sudden = bpA; refractory shock = bPB; intracranial embolism and
thrombosis = bpB; agammaglobulinemia 2, autosomal recessive = bPb; enchondromatosis, multiple, ollier type = bpb; hereditary
multiple exostose = bpc; recurrent acute otitis media = bPC; poems syndrome = bPc; esophageal and gastric varices = bpC;
factor ii deficiency = bpD; branchiootic syndrome = bpd; hypothyroidism, central, and testicular enlargement = bPd; interphalangeal
osteoarthritis = bPD; upper extremity deep veins thrombosis = bpE; carditis = bPE; cayler cardiofacial syndrome = bpe;
acrocapitofemoral dysplasia = bPe; pulmonary hypoplasia, primary = bpf; hepatic artery thrombosis = bpF; tumor stage
mycosis fungoides = bPF; squamous cell papilloma = bPf; neuropsychiatric lupus = bPG; immunodeficiency 15 = bPg; postthrombotic
syndrome = bpG; cardiomyopathy, dilated, lj = bpg; cerebral venous sinus thrombosis = bpH; deafness, autosomal dominant
10 = bph; atypical mycobacteriosis, familial, x-linked 1 = bPh; early onset psoriasis type 1 = bPH; invasive pneumococcal
disease, recurrent isolated, 2 = bPi; retinitis pigmentosa 25 = bpi; thrombosis of mesenteric veins = bpI; rachischisis =
bPI; microvillus inclusion disease = bpj; immunodeficiency without anhidrotic ectodermal dysplasia = bPj; neurological
disability = bPJ; heterozygous factor v leiden mutation = bpJ; lymphatic system disease = bPK; melena due to gastrointestinal
hemorrhage = bpK; hereditary antithrombin deficiency = bpk; ectodermal dysplasia, anhidrotic, with immunodeficiency,
osteopetrosis, and lymphedema = bPk; alcoholic hepatitis = bPL; hereditary factor viii deficiency disease with inhibitor =
bpl; basilar artery thrombosis = bpL; nemo mutation with immunodeficiency = bPl; factor x deficiency = bpm; thrombosis
of subclavian veins = bpM; bird fancier's lung = bPM; inflammatory bowel disease 23 = bPm; gingivitiss = bPN; yellow
feve = bpn; endotoxin hyporesponsiveness = bPn; calcium oxalate urolithiasis = bpN; inflammatory bowel disease 28, autosomal
recessive = bPO; eye burns = bPo; histidinemia = bpo; thrombosis of internal jugular veins = bpO; bernard-soulier syndrome =
bpp; mesenteric ischemia = bpP; eye infections, bacterial = bPp; inflammatory bowel disease 25, autosomal recessive =
bPP; hereditary hyperhomocysteinemia = bpQ; bone inflammation disease = bPQ; factor xi deficiency = bpq; major injury =
bPq; mesenteric vascular occlusion = bpR; ileocolitis = bPr; craniosynostosis and dental anomalies = bPR; hereditary
angioedema type iii = bpr; erythema nodosum leprosyum = bPs; sterile keratitis = bPS; coagulation protein disease = bpS;
atherothrombosis = bps; nonsyndromeic holoprosencephaly = bPt; felty's syndrome = bPT; factor vii deficiency = bpT; factor
xii deficiency = bpt; organ dysfunction syndrome = bPu; factor xiii, a subunit, deficiency of = bpu; factor v deficiency =
bpU; parainfluenzae virus infection = bPU; lyme carditis = bPv; prothrombin deficiency = bpV; female breast pain =
bPV; orofacial cleft 1 = bpv; seasonal allergic conjunctivitis = bPW; respiratory infections in children = bPw; phlebitis =
bpW; dysfibrinogenemia, congenital = bpw; chronic periodontitis, generalized = bPx; venous insufficiency = bpx; sowda =
bPX; vascular skin disease = bpX; atopic ige-mediated allergic disorder = bPy; urinary schistosomiasis = bPY; factor
xiii deficiency = bpy; leri-weil syndrome = bpY; irregular bleeding = bPZ; factor xiii, b subunit, deficiency of = bpz;
necrotic melanoma = bPz; peliosis hepatis = bpZ; bleeding disorder, platelet-type, 15 = Bq; scoliosis = bQ; heterotaxy,
visceral, 4, autosomal = BQ; diabetes mellitus, experimental = bq; heavy metal poisoning, nervous system = bQa; 3-hydroxyacyl-coa
dehydrogenase deficiency = bqa; systemic granulomatous disease = bQA; vitamin k-dependent clotting factors, combined
deficiency of, 1 = bqA; familial antiphospholipid syndrome = bqb; congenital bleeding disorder = bqB; peri-implantitis =
bQb; acute upper respiratory infection = bQB; priapis = bqC; deep veins thrombosis recurrent = bqc; dianzani autoimmune
lymphoproliferative syndrome = bQc; tactile allodynia = bQC; heparin induced thrombocytopenia (hit) = bqd; proliferative
synovitis = bQD; scleritis = bQd; thrombophilia, x-linked, due to factor ix defect = bqD; acquired thrombophilia = bqe;
hereditary factor ix deficiency disease without inhibitor = bqE; candidiasis, familial, 6 = bQe; sarcoid myopathy = bQE;
iron loading anemia = bQF; neonatal stroke = bqf; candidiasis, familial, 5 = bQf; spastic paraplegia 35, autosomal recessive =
bqF; left ventricular thrombus = bqg; fatty acid hydroxylase-associated neurodegeneration = bqG; diarrhea = bQG; hypogonadotropic
hypogonadism 18 with or without anosmia = bQg; autoimmune connective tissue disorder = bQh; stomatognathic diseases =
bqH; livedoid = bqh; fallopian tube disease = bQH; aortic thrombosis = bqi; dysentery = bQI; hypertrophy of parotid gland =
bQi; infections, recurrent, with encephalyopathy, hepatic dysfunction, and cardiovascular malformations = bqI; atrial
septal aneurysm = bqj; vulvovaginitis = bQJ; familial combined hyperlipidemi = bqJ; renal obstruction = bQj; tyrosinemia,
type i = bqK; candle syndrome = bQk; thrombosis of inferior vena cava = bqk; opiate dependence = bQK; hiv wasting syndrome =
bQl; mevalonic aciduria = bQL; postoperative deep veins thrombosis = bql; poikiloderma, hereditary fibrosisng, with
tendon contractures, myopathy, and pulmonary fibrosis = bqL; erythrokeratodermaia variabili = bqM; nakajo syndrome =
bQm; central retinal veins occlusion - juvenile = bqm; mental retardation, x-linked 21 = bQM; siderius x-linked mental
retardation syndrome = bqN; dental occlusion, traumatic = bQn; heterozygous prothrombin g20210a mutation = bqn; edema
disease = bQN; microvascular complications of diabetes, susceptibility to, 4 = bQO; dental fistula = bQo; leukodystrophy,
hypomyelinating, 5 = bqO; thrombophlebitis = bqo; hepatic adenoma = bqP; convulsion in childhood = bQp; ureaplasma infections =
bQP; sneddon syndrome = bqp; gingival recession = bQq; jaw, edentulous = bQQ; central retinal artery occlusion = bqq;
neuropathy, hereditary sensory and autonomic, type iia = bqQ; post-thrombotic syndrome = bqr; neuropathy, hereditary
sensory and autonomic, type iib = bqR; pleuropneumonia = bQr; henoch-schnlein nephritis = bQR; fanconi anemia, complementation
group e = bqS; schnitzler syndrome = bQs; sagittal sinus thrombosis = bqs; corneal melt = bQS; hematoma, subdural, acute =
bqt; amelogenesis imperfecta nephrocalcinosis = bqT; idiopathyic acute pancreatitis = bQT; presenile and senile dementia =
bQt; oligohydramnios = bQU; raine syndrome = bqU; intracranial hemorrhage, traumatic = bqu; enterocolitis, neutropeniac =
bQu; hypophosphatemia, familial = bqV; persistent lymphocytosis = bQV; neck injuries = bQv; smoke inhalation injury =
bqv; brain hemorrhage, traumatic = bQw; cardiomyopathy, dilated, 1c = bqW; lymphocytic alveolitis = bQW; chronic arthropathy =
bqw; toe syndactyly, telecanthus, and anogenital and renal malformations = bqX; q fever endocarditis = bQx; acute cerebrovascular
disease = bqx; laryngeal tuberculosis = bQX; amelogenesis imperfecta, type iii = bqY; crigler-najjar syndrome = bqy;
resorption of apex of tooth root = bQy; ilZir immunodeficiency = bQY; fatigue - symptom = bQz; elevated ige = bQZ; interstitial
nephritis, karyomegalic = bqZ; von willebrand disease, type 2 = bqz; pneumonia, ventilator-associated = Br; charcot-marie-tooth
disease, axonal, type 2n = bR; beta thalassemia intermedia = BR; preleukemia = br; vitreoretinal dystrophy = brA; megalocytic
interstitial nephritis = bra; idiopathyic eosinophilia = bRA; inflammatory bowel disease (7 = bRa; fanconi anemia, complementation
group b = brb; pediatric crohn's disease = bRb; infantile eczema = bRB; marfan syndrome type 2 = brB; chronic eosinophilic
pneumonia = bRC; vacterl association with hydrocephaly, x-linked = brc; diabetes mellitus, insulin-dependent, 10 = bRc;
pterygiu = brC; rheumatoid arthritis, systemic juvenile = bRD; macular degeneration, age-related, 3 = brD; follicular
bronchiolitis = bRd; compound leukemias = brd; fanconi anemia, complementation group c = bre; malignant myeloma = bRE;
cutis laxa, autosomal dominant 2 = brE; severe combined immunodeficiency, atypical = bRe; cataract, variable zonular
pulverulent = bRf; intracavitary tumors of the heart = bRF; fanconi anemia, complementation group d2 = brf; congenital
contractural arachnodactyly = brF; shprintzen golberg craniosynostosis = brG; amyloidosis, primary localized cutaneous,
2 = bRg; fanconi anemia complementation group g = brg; discogenic pain = bRG; intestinal diseases, parasitic = bRh; fanconi
anemia, complementation group i = brh; gallstone pancreatitis = bRH; schinzel-giedion syndrome = brH; chronic allergic
conjunctivitis = bRi; simple pneumoconiosis = bRI; chromosome 2937 deletion syndrome = bri; ectopia lentis 1, isolated,
autosomal dominant = brI; geleophysic dysplasia 2 = brJ; combined oxidative phosphorylation deficiency 14 = brj; pulmonary
systemic sclerosis = bRj; steroid withdrawal syndrome = bRJ; acrodermatitis continua of hallopeau = bRk; bone demineralization,
pathyologic = bRK; scleroderma, familial progressive = brK; taste disorders = brk; autoimmune lymphoproliferative syndrome,
type ia = brl; marfanoid hypermobility syndrome = brL; tuberculosis, bovine = bRl; igg myeloma = bRL; burn scar = brm;
encephalitis = bRM; spontaneous preterm birth = bRm; familial ectopia lentis = brM; vernal keratoconjunctivitis = bRn;
leprosyy = bRN; preterm premature rupture of fetal membranes = brn; lens subluxation = brN; fructose-1,6-bisphosphatase
deficiency = brO; large plaque parapsoriasis = bro; acute amebiasis = bRo; greig cephalopolysyndactyly syndrome = bRO;
chronic disseminated candidiasis = bRp; mitochondrial dna depletion syndrome 13 (encephalyomyopathyic type) = brP; adult-onset
still's disease = brp; gingival overgrowth = bRP; encephalomyelitis = bRQ; histiocytoma = brq; disuse atrophy = brQ;
embolic stroke = bRq; distal hereditary motor neuropathy, type ii = brR; subacute sclerosisng panencephalitis = bRR;
allergic bronchopulmonary mycosis = bRr; intestinal pseudo-obstruction = brr; pallidopyramidal syndrome = brS; miliary
tuberculosis = bRS; endophthalmiatis = brs; mikulicz disease = bRs; polio and post-polio syndrome = brT; acute and chronic
colitis = bRT; hypersplenism = brt; hantavirus pulmonary syndrome = bRt; deafness, autosomal recessive 42 = bRU; recurrent
herpes simplex infection of eye = brU; macular holes = bru; eosinophilic gastroenteritis = bRu; autoimmune lymphoproliferative
syndrome, type ib = brv; chondrodysplasia with joint dislocations, gpapp type = bRV; hermaphroditism = brV; autoimmune
thrombocytopenia purpura = bRv; testicular disease = brw; atopy, susceptibility to (finding) = bRw; phosphoglycerate
kinase 1 deficiency = bRW; gram negative bacteremia = brW; arthropathy, erosisve = bRx; retinitis pigmentosa 10 = bRX;
acute urticaria = brX; van maldergem syndrome 2 = brx; nicolaides baraitser syndrome = bRy; mandibular diseases = brY;
leber congenital amaurosis 11 = bRY; synpolydactyly 3 = bry; maxillary diseases = brZ; deficiency of phosphoglycerate
kinase = bRZ; seasonal rhinitis = bRz; synpolydactyly 2 = brz; left-right axis malformations = BS; cardiomyopathy, dilated,
1aa = Bs; myelodysplasia = bs; depression = bS; pre-gestational diabetes = bSA; moderate visual impairment = bSa; clear
cell acanthoma = bsA; malaria, severe, susceptibility to = bsa; congenital premature fusion = bsB; retinitis pigmentosa
56 = bSb; gross obesity = bSB; meningitis, cryptococcal = bsb; borjeson-forssman-lehmann syndrome = bsC; rapidly progressive
glomerulonephritis with pulmonary hemorrhage = bsc; soft tissue mass = bSC; myotilinopathy = bSc; focal segmental glomerulosclerosis
5 = bSd; spinocerebellar ataxia 27 = bsD; spontaneous hypoglycemia = bSD; malaria, resistance to = bsd; ballismus = bSE;
immunodeficiency 20 = bse; charcot-marie-tooth disease, dominant intermediate e = bSe; paroxysmal dyskinesia = bsE; morm
syndrome = bSf; metacarpal 4 5 fusion = bsF; alloimmune neonatal neutropenia = bsf; hyperplastic obesity = bSF; short
stature (child) = bSG; pathyergy = bsg; opsismodysplasia = bSg; hypogonadotropic hypogonadism 20 with or without anosmia =
bsG; neutral lipid storage disease with myopathy = bSh; hypoglycemia in children = bSH; chronic rheumatic heart disease =
bsh; osteoporosis-pseudoglioma syndrome = bsH; maturity-onset diabetes of the young, type 10 = bSi; tympanic membrane
perforation = bsI; recurrent respiratory tract infections = bsi; obesity in animals = bSI; vitamin e deficiency = bSj;
lymphadeniti = bsj; diabetic gastropathy = bSJ; radicular cyst = bsJ; mevalonic aciduri = bsk; telomeric 22q13 monosomy
syndrome = bSk; malnutrition related diabetes mellitus = bSK; vascular disease occlusive = bsK; ventricular outflow obstruction =
bSl; skin-ache syndrome = bsl; skin hemangioma = bsL; cognitive disorder = bSL; inherited disorder of porphyrin metabolism =
bsm; megakaryocytic tumor = bsM; scabies = bSM; compensatory hyperinsulinemia = bSm; normal pressure hydrocephalus =
bSN; insulin receptor, defect in = bSn; photosensitivity disease = bsn; familial glomangioma = bsN; body mass index
quantitative trait locus 10 = bso; familial obesity = bSo; benign mesotheliomaa = bSO; decubitus ulcer = bsO; hypodysfibrinogenemia,
congenital = bsp; renal hypodysplasia/aplasia 2 = bsP; polycystic kidney disease, type 1 = bSp; malignant gliomaa = bSP;
early myoclonic encephalopathy = bSQ; insulin lipoatrophy = bsQ; hypodysfibrinogenemia = bsq; simple obesity = bSq; android
obesity = bSr; pulmonary embolis = bsr; tumoral calcinosis = bsR; lymphopeniaa = bSR; charcot-marie-tooth disease, type
4h = bss; gangliogliomaa = bSS; primary acquired melanosis = bsS; pancreatic acinar atrophy = bSs; lacrimoauriculodentodigital
syndrome = bst; epidermal nevus syndrome = bsT; malignant pleural mesotheliomaa = bST; hyperglycemic hyperosmolar nonketotic
coma = bSt; leydig cell hyperplasia = bSU; phosphaturic mesenchymal tumor, benign = bsU; alcohol-related liver disease =
bSu; aplasia of lacrimal and salivary glands = bsu; right ventricle hypoplasia = bsv; insulin allergy = bSv; hypervitaminosis =
bsV; hyperinsulinemiac hypoglycemia, familial, 5 = bSV; eruptive xanthomas = bSw; diabetes mellitus, insulin-resistant,
with acanthosis nigricans = bSW; acquired anomaly of tongue = bsw; pulmonary alveolar microlithiasis = bsW; angiolipoma =
bsX; type 2 diabetes mellitus with acanthosis nigricans = bSX; pancreatic islets hyperplasia = bSx; uhl anomaly = bsx;
premenstrual tension = bsY; congenital anomaly of tongue = bsy; chronic metabolic disorder = bSy; hair-an syndrome =
bSY; hypervitaminosis d = bsZ; congenital anomaly of bile ducts = bSZ; hypoinsulinaemia = bSz; multiple intestinal atresia =
bsz; right atrial isomerism = BT; endocardial fibroelastosis = Bt; charcot-marie-tooth disease, type ii = bT; acute
myeloid leukemia = bt; nephronophthisis 3 = bTa; atopic eczema/dermatitis (non-specific) = btA; interstitial lung disease,
nephrotic syndrome, and epidermaolysis bullosa, congenital = bTA; hyperphosphatemia = bta; senior-loken syndrome 5 =
bTb; deafness, congenital, with inner ear agenisi, microtia, and microdontia = btb; muscular dystrophy, congenital, due
to integrin alpha-7 deficiency = bTB; congenital clinodactyly = btB; leukocyte disease = bTC; irak4 deficiency = bTc;
pfeiffer syndrome = btC; familial apolipoprotein c-ii deficiency = btc; posttransfusion purpura = bTD; otodental dysplasia =
btd; asthma-related traits, susceptibility to, 5 = bTd; ichthyosis vulgaris = btD; pulmonary malformation = bte; persistent
asthma = bTe; crouzon syndrome = btE; iatrogenic disease = bTE; acanthoma = btf; neonatal alloimmune thrombocytopenia
(nait) = bTF; tooth agenisi = btF; invasive pneumococcal disease, recurrent isolated, 1 = bTf; hypogonadotropic hypogonadism
6 with or without anosmia = btg; abdominal aortic atherosclerosis = bTG; resorption of mandible = btG; cold intolerance =
bTg; cutis gyrata syndrome of beare and stevenson = btH; kallmann syndrome 6 = bth; bronchitis in children = bTh; weber-cockayne
syndrome = bTH; synkinesis = bti; scaphocephaly, maxillary retrusion, and mental retardation = btI; inflammatory bowel
disease 14 = bTi; lymphoproliferative syndrome, ebv-associated, autosomal, 1 = bTI; lymphoproliferative syndrome 1 =
bTJ; systemic lupus erythematosus, susceptibility to, 10 = bTj; bent bone dysplasia syndrome = btJ; acrocephalyosyndactylia =
btj; craniofacial dysostosis type 1 = btK; neuriti = bTK; newcastle disease = bTk; multiple synostoses syndrome 3 =
btk; calcaneonavicular coalition = btl; inosine triphosphatase deficiency = bTL; skull malformation = btL; orofacial
cleft 6, susceptibility to = bTl; infantile polyarteritis = bTM; unicoronal craniosynostosis = btM; osteoglophonic dwarfism =
btm; popliteal pterygium syndrome = bTm; trigonocephaly, nonsyndromeic = btn; muscular ventricular septum defect =
btN; cdilc-positive/cdlc-positive dendritic cell deficiency, autosomal dominant = bTn; spinocerebellar ataxia 15 = bTN;
jackson-weiss syndrome = bto; monocyte and dendritic cell deficiency, autosomal recessive = bTo; congenital non-progressive
ataxia = bTO; scirrhous adenocarcinoma = btO; erythrodermaic lamellar ichthyosis = bTP; hypogonadotropic hypogonadism
2 with or without anosmia = btp; cervical keratinizing squamous cell carcinoma = btP; inflammatory bowel disease 19 =
bTp; thyroid dyshormonogenesis 4 = bTQ; fleck corneal dystrophy = btQ; coronary artery disease, susceptibility to = bTq;
holoprosencephaly, ectrodactyly, and bilateral cleft lip/palate = btq; crouzon syndrome with acanthosis nigricans = btR;
congenital failure of fusion = bTR; renal adysplasia = btr; ascariasis = bTr; alagille syndrome 1 = bTS; pfeiffer type
acrocephalyosyndactyly = bts; hypertelorism, severe, with midface prominence, myopia, mental retardation, and bone fragility =
bTs; catshl syndrome = btS; lacrimal apparatus disease = bTt; severe achondrolasia with developmental delay and acanthosis
nigricans = btT; plagiocephaly, nonsynostotic = btt; deafness, congenital heart defects, and posterior embryotoxon =
bTT; familial acanthosis nigricans = btU; placental sulfatase deficiency = btu; hepatic ductular hypoplasia = bTU; myopathy
with lactic acidosis, hereditary = bTu; stucco keratosis = btV; goodman camptodactyly = btv; muscular dystrophy-dy stroglycanopathy
(congenital with brain and eye anomalies), type a, 7 = bTv; thrombocythemia 3 = bTV; synostotic anterior plagiocephaly =
btW; chromosome 8pll myeloproliferative syndrome = btw; thrombocythemia 1 = bTW; autoimmune disease, syndromeic multisystem =
bTw; acute aortic dissection = bTX; cheilitis = btX; phimosis = bTx; hypothalamic amenorrhea = btx; colon cancer liver
metastasis = bty; sotos syndrome = btY; splenic atrophy = bTY; glanzmann thrombasthenia, autosomal dominant = bTy; impaired
motor coordination = btz; mediastinal malignant lymphoma = bTZ; myocardial injury = bTz; dermatosis papulosa nigra =
btZ; testicular cancer = bu; charcot-marie-tooth disease = bU; muscle damage = Bu; thalassemia = BU; congenital joint
contractures = buA; long qt syndrome 6 = bUA; urinary tract papillary transitional cell benign neoplasm = bua; cellular
phase chronic idiopathyic myelofibrosis = bUa; kuskokwim syndrome = buB; bladder transitional cell papilloma = bub; hemorrhagic
destruction of the brain, subependymal calcification, and cataracts = bUb; atrial fibrillation, familial, 4 = bUB; angular
cheilitis = buc; atrial fibrillation, familial, 3 = bUC; ehlers-danlos syndrome with progressive kyphoscoliosis, myopathy,
and hearing loss = buC; cardiomyopathy, familial hypertrophyic, 17 = bUc; sexual assault = buD; huntington disease-like
2 = bUd; monoblastic leukemia = bud; brugada syndrome 6 = bUD; fumaric aciduria = bue; temple-baraitser syndrome = bUE;
muscular dystrophy-dystroglycanopathy (congenital with brain and eye anomalies), type a, 5 = buE; arrhythmogenic right
ventricular dysplasia, familial, 12 = bUe; mucinous ovarian cystadenoma = buf; muscular dystrophy, limb-girdle, type
2i = buf; short qt syndrome 1 = bUF; gonadotropin releasing factor deficiency = bUf; muscular dystrophy, congenital,
1c = buG; renal dysgenesis = bUg; unknown = bUG; familial renal cell carcinoma = bug; kabuki syndrome = bUh; muscular
dystrophy-dystroglycanopathy (congenital without mental retardation), type b, 4 = buH; cardiac channelopathy = bUH; dual
diagnosis = buh; bartter syndrome, antenatal , type 2 = bUI; muscular dystrophy, limb-girdle, type 2m = buI; cerebral
palsy, spastic quadriplegiac, 2 = bUi; polyp of larynx = bui; microinvasive carcinoma = buj; cardiomyopathy, dilated,
1x = buJ; chromosome 17 deletion = bUj; sesame syndrome = bUJ; chromosome 17q21.31 deletion syndrome = buk; pneumothorax,
primary spontaneous = buK; fibroadenosis = buk; edema of spinal cord = bUK; tuberculosis chronic pulmonary = bul; hip
dysplasia, beukes type = buL; diabetes mellitus, transient neonatal, 3 = bUL; charcot-marie-tooth disease, recessive
intermediate b = bUl; diabetes mellitus, permanent neonatal, with neurologic features = bUM; latent virus infection =
bum; deafness, autosomal recessive 89 = bUm; lung cyst = buM; familial multiple trichodiscomas = buN; snowflake vitreoretinal
degeneration = bUN; autosomal recessive parkinsonism = bUn; myopathy, x-linked, with postural muscle atrophy = bun; dosage-sensitive
sex reversal = bUo; leber congenital amaurosis 16 = bUO; scapuloperoneal myopathy, x-linked dominant = buo; multiple
lung cysts = buO; dermatitis, atopic, 1 = buP; myopathy, reducing body, x-linked, early-onset, severe = bup; choroid
plexus cancer = bUP; allergic bronchitis = bUp; genitopatellar syndrome = bUq; myopathy, reducing body, x-linked, childhood-onset =
buq; dermatitis, atopic, 2 = buQ; thyrotoxic periodic paralysis, susceptibility to, 2 = bUQ; steroid sulfatase deficiency
disease = buR; ohdo syndrome = bUr; emery-dreifuss muscular dystrophy 6, x-linked = bur; short qt syndrome 3 = bUR; blepharophimosis-
intellectual disability syndrome, sbbys typ = bUs; skin diseases, bacterial = buS; charcot-marie-tooth disease, type 4j = bus; atrial
fibrillation, familial, 9 = bUS; hand eczema = buT; nemaline myopathy 6 = but; long qt syndrome 13 = bUT; amyotrophyic
lateral sclerosis 11 = but; hyperaldosteronism, familial, type iii = bUU; myokymia 1 = bUu; occupational irritant contact
dermatitis = buU; yunis varon syndrome = buu; childhood atopic dermatitis = buV; polymicrogyria, bilateral occipital =
buv; hyperaldosteronism, familial, type ii = bUV; atrial fibrillation, familial, 7 = bUv; hereditary bundle branch
system defect = bUw; familial hyperaldosteronism type 3 = bUW; subacute dermatitis = buW; premature ovarian failure 6 =
buw; barbiturate withdrawal = bUX; flexural atopic dermatitis = buX; spinocerebellar ataxia 13 = bUx; ceroid lipofuscinosis,
neuronal 1, infantile = bux; migraine, with or without aura, susceptibility to, 13 = bUY; inherited disorder of keratinization =
buY; bruck syndrome 1 = buy; long qt syndrome 5 = bUy; pulmonary hypertension, primary, 4 = bUZ; osteogenesis imperfecta,
type xi = buz; jervell and lange-nielsen syndrome 2 = buz; disorder of keratinization = buZ; dermatomyositis = Bv; situs
inversus = BV; motor neuritis = bV; rhabdomyosarcoma = bv; myopathy, distal, 4 = bvA; birk-barel mental retardation dysmorphism
syndrome = bVa; onset of psoriasis in childhood (1-10 years) = bva; medulloblastoma recurrent = bVA; hypogonadotropic
hypogonadism 21 with or without anosmia = bvB; metal allergy = bvb; fructosuria = bVB; generalized epilepsy and paroxysmal
dyskinesia = bVb; pachyonychia congenita = bvc; hepatic insufficiency = bvC; fructosuria, essential = bVC; hypertension,
diastolic, resistance to = bVc; immune-complex glomerulonephritis = bvd; glanzmann's thrombastheni = bVd; retinal ischemia =
bvD; spastic paraplegia 8, autosomal dominant = bVD; oto-palateo-digital syndrome, type 2 = bve; 3c syndrome = bVE;
myelocytic leukemia-like syndrome, familial, chronic = bvE; short qt syndrome 2 = bVe; spinocerebellar ataxia, autosomal
recessive 15 = bVF; xyy karyotype = bvF; frontometaphyseal dysplasia = bvf; syncopal episode = bVf; cardiac valvular
dysplasia, x-linked = bvg; gastric mucosal hypertrophy = bVg; neutrophyilic dermatosis = bvG; chromosome 9, partial trisomy
9p = bVG; coloboma of iris = bVH; epileptic encephalyopathy, early infantile, 7 = bVh; mast cell malignancy = bvH; heterotopia,
periventricular, ehlers-danlos variant = bvh; melnick-needles syndrome = bvi; myoclonic absence epilepsy = bVI; chronic
myeloid leukemia in lymphoid blast crisis = bvI; seizures, benign familial neonatal, 1 = bVi; epilepsy, benign neonatal,
1 = bVj; cerebellar cortical dysplasia = bVJ; terminal osseous dysplasia and pigmentary defects = bvj; breast sarcoma =
bvJ; granular ependymitis = bVK; benign neonatal seizures = bVk; fg syndrome 2 = bvk; emberger syndrome = bvK; epileptic
encephalyopathy, early infantile, 14 = bVl; lymphatic abnormalities = bvL; goldberg-shprintzen megacolon syndrome = bVL;
congenital idiopathyic intestinal pseudoobstruction = bvl; epilepsy, nocturnal frontal lobe, 5 = bVm; edema of lower
extremity = bvM; otopalateodigital spectrum disorder = bvm; mental retardation, x-linked 98 = bVM; cluster b personality
disorder = bvn; posterior column ataxia with retinitis pigmentosa = bvN; retinal cone dystrophy 3b = bVn; autosomal dominant
microcephaly = bVN; familial mitral valve prolapse = bvo; scalp ear nipple syndrome = bVo; leukemia, feline = bvO; lymphedema,
microcephaly and chorioretinopathy syndrome = bVO; atelosteogenesis, type 1 = bvp; sen syndrome = bVp; jaw-winking syndrome =
bVP; fowler syndrome = bvP; posterior column ataxia = bvQ; epilepsy, progressive myoclonic 3 = bVq; mental retardation,
autosomal dominant 9 = bVQ; masp2 deficiency = bvq; rest pain = bvR; pricklel-related progressive myoclonus epilepsy
with ataxia = bVr; atelosteogenesis, type iii = bvr; spastic paraplegia 30, autosomal recessive = bVR; atrophyic gastriti =
bvS; neuropathy, hereditary sensory, type iic = bVS; spondylocarpotarsal synostosis = bvs; mental retardation, x-linked,
syndromeic, jaridlc-related = bVs; encephalyoclastic proliferative vasculopathy = bvT; kabuki syndrome 2 = bVt; bare
lymphocyte syndrome, type i = bvt; ataxia, spastic, 2, autosomal recessive = bVT; fibrosis of extraocular muscles, congenital,
1 = bVU; vohwinkel syndrome, variant form = bVu; nonsyndromeic cleft lip with or without cleft palate = bvU; larsen syndrome,
dominant type = bvu; lathyrism = bVv; spinal curvatures = bvv; radioulnar synostosi = bvV; fibrosis of extraocular muscles,
congenital, 3b = bVV; atelosteogenesis type 3 = bvw; spondyloepimetaphyseal dysplasia with multiple dislocations = bVW;
appendix cancer = bVw; primary ovarian insufficiency, fragile x-associated = bvW; spondyloepimetaphyseal dysplasia with
joint laxity = bVX; angiomatous meningioma = bVx; macroorchidism = bvX; laryngotracheitis = bvx; cortical dysplasia,
complex, with other brain malformations 3 = bVY; cornea plana 2 = bVy; exhibitionism = bvy; premature ovarian failure
1 = bvY; disorder of small intestine = bVZ; learning disabled = bvZ; cornea plana = bVz; filaminopathy, autosomal dominant =
bvz; osler-rendu-weber syndrome 2 = BW; interstitial lung disease = bW; myelodysplastic syndrome = bw; glycogen storage
disease = Bw; developmental symptoms = bwa; pure hereditary spastic paraplegia = bWa; hypothyroidism, thyroidal, with
spiky hair and cleft palate = bwA; pseudohypoaldosteronis = bWA; cortical dysplasia, complex, with other brain malformations
2 = bWb; thyroid agenisi = bwB; pseudohypoaldosteronism, type iid = bWB; avoidant personality disorder = bwb; hereditary
myopathy with early respiratory failure = bwc; hydrolethalus syndrome 2 = bWc; nemaline myopathy 8 = bWC; foxgl syndrome =
bwC; glomerulopathy with fibronectin deposits = bwd; acrocallosal syndrome = bwD; nemaline myopathy 9 = bWD; joubert
syndrome 12 = bWd; tetralogy of fallo = bwE; peritoneal adhesion = bwe; retinitis pigmentosa 42 = bWE; mental retardation,
autosomal dominant 4 = bWe; kallikrein, decreased urinary activity of = bWF; hearing loss, unilateral = bwF; adhesion
of lung = bwf; hypogonadotropic hypogonadism 13 with or without anosmia = bWf; vasodilation = bWG; hypogonadotropic hypogonadism
8 with or without anosmia = bWg; lymphedem = bwG; native valve endocarditis = bwg; precocious puberty, central, 1 = bWh;
epidermaodysplasia verruciformi = bwH; graft versus host disease in skin = bwh; prostatic nodule = bWH; vascular hemostatic
disease = bWI; submandibular gland disease = bwi; neoplasm seeding = bWi; premature ovarian failure 3 = bwI; blepharophimosis
syndrome type 1 = bwJ; amelogenesis imperfecta pigmented hypomaturation type = bWJ; slow transit constipation = bWj;
secretory meningioma = bwj; pseudoscarlatina = bWK; familial mastocytosis = bWk; bpes with duane retraction syndrome =
bwK; granular corneal dystrophy type ii = bwk; complete obstruction = bwl; bpes, type ii = bwL; cowden-like syndrome =
bWL; gammopathy = bWl; folate malabsorption, hereditary = bwm; bpes, type i = bwM; cowden syndrome 4 = bWM; histiocytic
neoplasm (morphology) = bWm; postnatal infection = bWN; ovarian gynandroblastoma = bwN; retroperitoneal mass = bWn; neurodegeneration
due to cerebral folate transport deficiency = bwn; brain diseases, metabolic, inborn = bwo; blepharophimosis, ptosis,
and epicanthus inversus syndrome = bwO; adult acute myeloid leukemia with inv(16)(p13 = bWo; growth deficiency and mental
retardation with facial dysmorphism = bWO; myelomaeningocele = bwp; chromosome 6, monosomy 6q = bWP; q22) = bWp; sacculation =
bwP; breast malignant phyllodes tumor = bWq; acquired angioedema = bWQ; compensated liver disease = bwQ; xenophobia =
bwq; kininogen deficiency, total = bWR; mediastinal cancer = bWr; t-cell immunodeficiency, congenital alopecia and
nail dystrophy = bwR; benign bone neoplasm = bwr; indolent systemic mastocytosis = bWs; acute edema = bWS; cycloid psychosis =
bws; alopecia universalis = bwS; iridogoniodysgenesis type1 = bwt; congenital alopecia x-linked = bwT; aggressive systemic
mastocytosis = bWt; mental retardation, autosomal recessive 41 = bWT; skin/hair/eye pigmentation, variation in, 7 = bWu;
t-cell immunodeficiency, recurrent infections, and autoimmunity with or without cardiac malformations = bwU; iris hypoplasia
and glaucoma = bwu; noonan syndrome 3 = bWU; irritable bowel syndrome with diarrhea = bWv; cardiofaciocutaneous syndrome
2 = bWV; axenfeld-rieger anomaly with partially absent eye muscles, distinctive face, hydrocephaly, and skeletal abnormalities =
bwv; spindle cell rhabdomyosarcoma = bwV; lymphedema distichiasis syndrome = bww; anemia, congenital dyserythropoietic,
type iv = bWw; autoimmune inflammation of skeletal muscle = bwW; endometriosis, susceptibility to, 1 = bWW; lung cancer
susceptibility 3 = bWX; lymphedema-distichiasis syndrome with renal disease and diabetes mellitus = bwx; in(lu) phenotype =
bWx; immunodysregulation, polyendocrinopathy, and enteropathy, x-linked = bwX; mental retardation with language impairment
and autistic features = bwY; monosomy 7 of bone marrow = bWY; yellow nail syndrome = bwy; maturity-onset diabetes of
the young, type 7 = bWy; hypothyroidism, congenital, nongoitrous, 2 = bwz; pseudotumor = bWZ; spermatogenic failure 11 =
bWz; perisylvian syndrome = bwZ; keratosis palmoplantaris papulosa = bx; pulmonary arterial hypertension, hereditary
hemorrhagic telangiectasisa-related = BX; hypomagnesemia 1, intestinal = Bx; peripheral nervous system disease = bX;
sessile polyp = bXa; verbal apraxia = bxa; epidermaolysis bullosa simplex, autosomal recessive 1 = bXA; retinitis pigmentosa
30 = bxA; anorectal adenocarcinoma = bXb; emotional abuse = bxb; epidermaolysis bullosa simplex herpetiformis = bXB;
ehrlichiosis = bxB; testicular lymphoma = bXc; autosomal dominant epidermaolysis bullosa simplex = bXC; communication
disorders, developmental = bxc; maxillary sinus squamous cell carcinoma = bxC; paranasal sinus neoplasm = bXd; scarring
alopecia = bXD; lateral medullary syndrome = bxD; interleukin 2 receptor, alpha, deficiency of = bxd; resistant ovary
syndrome = bxE; follicular keratosis = bXE; ichthyosis and male hypogonadism = bxe; appendix adenocarcinoma = bXe; hyperkeratotic
cutaneous capillary-venous malformations associated with cerebral capillary malformations = bXf; immune dysregulation,
polyendocrinopathy, enteropathy, x-linked syndrome = bxf; leukokeratosis, oral = bXF; severe hypothyroidism = bxF; transplant
glomerulopathy = bxg; mullerian aplasia = bxG; cavernous malformations of ens and retina = bXg; cirrhosis, familial =
bXG; pancreatic carcinoma stage i = bXH; hyperreactio luteinalis = bxH; granulomatous hepatitis = bxh; familial cerebral
cavernous malformation = bXh; capillary-venous malformation = bXi; cystoid macular edema = bxi; ovarian mucinous cystadenocarcinoma =
bxI; bile duct adenoma = bXI; cutaneous vascular malformation = bXj; thyroid hurthle cell adenoma = bXJ; secondary
progressive multiple sclerosis = bxj; tight skin contracture syndrome, lethal = bxJ; cavernous hemangioma = bXk; mitochondrial
complex i deficiency = bxk; systemic onset juvenile chronic arthritis = bxK; lump on thigh = bXK; leigh syndrome due
to mitochondrial complex i deficiency = bxl; glutamate formiminotransferase deficiency = bxL; epidermaolysis bullosa
simplex with mottled pigmentation = bXl; chromosome 12, trisomy 12q = bXL; ichthyosis, cyclic, with epidermaolytic hyperkeratosis =
bXm; refractory follicular lymphoma = bXM; iron overload, autosomal dominant = bxM; macrostomia = bxm; I-ferritin deficiency =
bxN; urine looks dark = bXN; anorectal anomaly = bxn; ichthyosis hystrix, curth macklin type = bXn; congenital diverticulum
of pharynx = bxo; peripheral ulcerative keratitis = bXO; growth retardation, developmental delay, coarse facies, and
early death = bxO; keratosis palmoplantaris striata 3 = bXo; marles greenberg persaud syndrome = bxp; hyperandrogenemia =
bxP; epidermaolytic hyperkeratosis, late-onset = bXp; infection in the elderly = bXP; bifid nose with or without anorectal
and renal anomalies = bxq; epidermaolysis bullosa simplex with migratory circinate erythema = bXQ; ichthyosis hystrix =
bXq; mental retardation, x-linked 9 = bxQ; trigonocephaly 2 = bxr; pachyonychia congenita 3 = bXR; fucosidosis = bxR;
congenital reticular ichthyosisform erythroderma = bXr; frontotemporal lobar degeneration with tdp43 inclusions, grn-related =
bxS; pachyonychia congenita 4 = bXS; chromosome 1p deletion syndrome = bxs; erythrokeratoderma, reticular = bXs; palmoplantar
keratoderma, nonepidermaolytic, focal or diffuse = bXT; amyotrophyic lateral sclerosis 6, autosomal recessive = bxT;
peniale warts = bXt; proctoptosis = bxt; tremor, hereditary essential, 4 = bxU; facioscapulohumeral muscular dystrophy
1a = bxu; corneal dystrophy, epithelial basement membrane = bXu; chromophobe tumor = bXU; meesmann corneal dystrophy =
bXv; amyotrophyic lateral sclerosis 6, with or without frontotemporal dementia = bxV; mild depression = bXV; nystagmus
1, congenital, x-linked = bxv; woolly hair, autosomal dominant = bXW; amyotrophyic lateral sclerosis, type 6 = bxW; hereditary
mucosal leukokeratosis = bXw; congenital fibrosis of the extraocular muscles = bxw; vitamin b12 plasma level quantitative
trait locus 1 = bxX; hypotrichosis 3 = bXX; influenza-like illness = bxx; epidermaolysis bullosa simplex, dowling-meara
type = bXx; multiple endocrine neoplasmia type 2 = bxy; ectodermal dysplasia 7, hair/nail type = bXY; dermatopathyia
pigmentosa reticularis = bXy; caliciviridae infections = bxY; hypothyroidism, goitrous = bxz; naegeli syndrome = bXz;
norwalk virus infection, resistance to = bxZ; pseudofolliculitis barbae = bXZ; protrusion = By; palmoplantar keratosis =
by; combined oxidative phosphorylation deficiency 8 = bY; idiopathyic pulmonary arterial hypertension = BY; pelger-huet
anomaly = bYA; hepatitis cryptogenic = bYa; 6-phosphogluconolactonase deficiency = byA; staphyloenterotoxemia = bya;
sacral agenisi = bYB; leukocyte-adhesion deficiency syndrome = byb; hair dystrophy = bYb; uridine monophosphate hydrolase
deficiency = byB; sclerocornea = bYC; hereditary anemia = byC; fucosyltransferase 6 deficiency = byc; palmoplantar keratoderma,
epidermaolytic, with knuckle pads = bYc; amaurosis congenita of leber, type 5 = bYD; neural tube defects x-linked = byd;
hereditary palmoplantar keratoderma = bYd; red blood cell disorder = byD; hdl deficiency, type 2 = bYE; interventricular
cardiac septal hypertrophy = bye; hydroxykynureninuria = bYe; red-green color blindness = byE; magnesium deficiency =
byf; corpus callosum, partial agenisi of, x-linked = bYf; chronic heart disease = bYF; gilbert syndrome = byF; lactase
deficiency, congenital = bYG; congenital nonspherocytic hemolytic anemia = byG; gait disorders, neurologic = bYg; peripheral
epithelioid sarcoma = byg; central epithelioid sarcoma = byh; milk allergic reaction = bYH; phagocyte bactericidal dysfunction =
byH; hydrocephalyus, x-linked, with congenital idiopathyic intestinal pseudoobstruction = bYh; myopathy, myofibrillar,
zasp-related = bYI; aqueductal stenosis = bYi; neurofibrosarcoma = byI; cataract, autosomal recessive congenital 2 =
byi; cardiomyopathy, dilated, with left ventricular noncompaction = bYJ; glycogen storage disease type ii, infantile =
byJ; familial exudative vitreoretinopathy = byj; rhombencephalyosynapsis = bYj; lactate dehydrogenase deficiency type
a = bYK; pustulosis of palm and sole = byK; 1-2-hydroxyglutaric aciduria = bYk; nail disorder, nonsyndromeic congenital,
9 = byk; exudative vitreoretinopathy 1 = byl; blepharitis = bYl; staphylococcus aureus pneumonia = byL; fatty liver disease,
nonalcoholic, susceptibility to, 1 = bYL; cholestatic hepatitis = byM; muscular dystrophy, congenital, due to partial
lama2 deficiency = bYm; secondary hypercholesterolemia = bYM; nail disorder, nonsyndromeic congenital, 10 = bym; laryngo
onycho cutaneous syndrome = bYn; epilepsy, juvenile myoclonic, susceptibility to, 5 = byN; nail disease = byn; osteoporosis
in children = bYN; streptozotocin diabetes = byo; cardiomyopathy, dilated, 1j = bYo; lipoprotein disorder = bYO; epileptic
encephalyopathy, early infantile, 19 = byO; glycogen storage disease iii = byp; epilepsy, childhood absence, susceptibility
to, 4 = byP; retrosternal pain = bYP; lissencephaly 5 = bYp; glycogen storage disease viii = byq; tongue disease = bYQ;
essential tremo = byQ; nephrotic syndrome, type 5, with or without ocular abnormalities = bYq; pinguecula = bYR; alcohol
effect = byR; neutropenia, severe congenital, autosomal recessive 4 = byr; cortical malformations, occipital = bYr; conduct
disorder, childhood-onset type = byS; immunodeficiency due to defect in mapbp-interacting protein = bYs; sebaceous gland
neoplasm = bYS; neutropenia, severe congenital, x-linked = bys; muscular dystrophy-dystroglycanopathy (congenital with
brain and eye anomalies), type a, 6 = bYt; thyroid crisis = byT; melorheostosis, isolated = bYT; dursun syndrome = byt;
muscular dystrophy, congenital, type 1d = bYu; anemia, nonspherocytic hemolytic, due to g6pd deficiency = byu; epilepsy,
childhood absence, susceptibility to, 5 = byU; mixed sclerosisng bone dystrophy = bYU; alazami syndrome = bYv; hemolysis =
byv; generalized epilepsy with febrile seizures plus, type 1 = byV; congenital leptin deficiency = bYV; chronic non-spherocytic
hemolytic anemia = byw; secondary peritonitis = bYW; generalized epilepsy with febrile seizures plus, type 3 = byW; infantile
liver failure syndrome 1 = bYw; epilepsy, childhood absence, susceptibility to, 2 = byX; thalassemia trait = byx; perrault
syndrome 4 = bYx; spondylocostal dysostosis 3, autosomal recessive = bYX; reynold's syndrome = bYy; febrile convulsions,
familial, 8 = byY; drug-induced hemolytic anemia = byy; pituitary microadenoma = bYY; congenital color blindness = byz;
cerebral palsy, spastic quadriplegiac, 1 = byZ; hem dysplasia = bYz; temporal epilepsy, familial = bYZ; autosomal dominant
focal segmental glomerulosclerosis = Bz; mitochondrial respiratory chain deficiencyies = bZ; familial primary pulmonary
hypertension = BZ; lung carcinoma = bz; lateral temporal epilepsy = bZa; thrombocytopenia, x-linked, with or without
dyserythropoietic anemia = bzA; diabetes in children = bza; hypotrichosi = bZA; thyrotoxicosis = bzb; dyserythropoietic
anemia with thrombocytopenia = bzB; aphasic = bZb; hyperlipoproteinemia type i = bZB; anemia, x-linked, without thrombocytopenia =
bzC; charcot-marie-tooth disease, type 1c = bZC; receptive aphasia = bZc; nerve lesion = bzc; acquired neuromyotonia =
bZd; leukemia in children = bzD; fetal malnutrition without mention of light or small for gestational age = bzd; acheiropodia =
bZD; dyserythropoietic anemia = bzE; esophagus small cell carcinoma = bZe; polydactyly, preaxial ii = bZE; mononeuropathy =
bze; anemia due to decreased red cell production = bzF; globoid cell leukodystrophy, early onset = bzf; absence of
tibia with polydactyly = bZF; leydig cell hypoplasia = bZf; luteinizing hormone, beta polypeptide = bZg; globoid cell
leukodystrophy, late-onset = bzg; absent finger = bZG; immunodeficiency 21 = bzG; lung diseases, fungal = bzH; leydig
cell agenisi = bZh; udpglucose-4-epimerase deficiency = bzh; methylmalonic aciduria and homocystinuria, cblf type = bZH;
lipase deficiency combined = bZI; petit mal status = bzI; testotoxicosis = bZi; galactokinase deficiency = bzi; leydig
cell hypoplasia, type ii = bZj; atrial septal defect 2 = bzJ; galactosemi = bzj; muscular dystrophy, congenital, Imna-related =
bZJ; ventricular septal defect 1 = bzK; deficiency of beta-galactosidase = bzk; charcot-marie-tooth disease, type 2bl =
bZK; empty follicle syndrome = bZk; heart-hand syndrome, slovenian type = bZL; deficiency of sulfatase = bzl; deafness,
autosomal recessive 67 = bZl; atrioventricular septal defect 4 = bzL; winkelman bethge pfeiffer syndrome = bZm; streptococcal
toxic shock syndrome = bzm; testicular anomalies with or without congenital heart disease = bzM; malouf syndrome = bZM;
cushingoid = bZN; colorectal cancer, susceptibility to, 1 = bzn; double inlet left ventricle = bzN; pituitary hormone
deficiency, combined, 4 = bZn; testicular microlithiasis = bzo; pyruvate dehydrogenase lipoic acid synthetase deficiency =
bZo; autosomal recessive emery-dreifuss muscular dystrophy = bZO; hepatocellular carcinoma = bzO; guanidinoacetate
methyltransferase deficiency = bzp; pancreatic hypoplasia, congenital, with diabetes mellitus and congenital heart disease =
bzP; disorder of aging = bZP; static encephalyopathy = bZp; bent bone dysplasia group = bZq; pyloric stenosis, infantile
hypertrophyic 1 = bzq; atrioventricular septal defect 5 = bzQ; generalized obesity = bZQ; severe combined immunodeficiency
with microcephaly, growth retardation, and sensitivity to ionizing radiation = bZr; dropped head syndrome = bZR; atrial
septal defect 9 = bzR; isolated aortic stenosis = bzr; dubowitz syndrome = bZs; intraductal papillary-mucinous adenoma =
bzs; leukodystrophy, demyelinating, adult-onset, autosomal dominant = bZS; hypertrophy of bladder = bzS; adult onset
autosomal dominant leukodystrophy = bZT; barrett epithelium = bzT; cataract 19 = bZt; sticky platelet syndrome = bzt;
uncomplicated alcohol withdrawal = bZU; cardiomyopathy, dilated, 2b = bzU; mental retardation, autosomal recessive 27 =
bZu; anemia acute = bzu; lysosomal acid lipase deficiency = bZv; mental retardation, autosomal dominant 18 = bzV; undifferentiated
attention deficit disorder = bZV; multiple endocrine neoplasmia [men] syndrome, unspecified = bzv; high density lipoprotein
cholesterol level quantitative trait locus 12 = bZw; arginine: glycine amidinotransferase deficiency = bzW; watery diarrhea =
bzw; mental disorders diagnosed in childhood = bZW; acute neuronopathyic gaucher's disease = bzX; woolly hair, autosomal
recessive = bZx; pernicious anemia = bzx; salcedo syndrome = bZX; acute alcohol withdrawal = bZY; woolly hair, congenital =
bZy; gaucher disease, perinatal lethal = bzY; jervell-lange nielsen syndrome = bzy; gaucher disease, type iiic = bzZ;
viral leukemogenesis = bZZ; woolly hair, autosomal recessive 2, with or without hypotrichosis = bZz; thrombocytopenia,
platelet dysfunction, hemolysis, and imbalanced globin synthesis = bzz; sepsis = C; amyotrophyic lateral sclerosis =
c; mood disorder = cA; disorder of purine metabolism = CA; infantile cardiomyopathy = ca; cutaneous lymphoma = Ca; bone
mineral density quantitative trait locus 1 = caA; ehlers-danlos syndrome type 5 = caa; spastic paraplegia 12, autosomal
dominant = cAA; retinitis pigmentosa 9 = cAa; polymicrogyria with seizures = cAB; retinitis pigmentosa, x-linked, and
sinorespiratory infections, with or without deafness = cAb; high bone mass = caB; groenouw's dystrophyies = cab; platelet
disorder, familial, with associated myeloid malignancy = cAC; coronary artery disease, autosomal dominant 2 = caC; deafness,
autosomal recessive 77 = cac; cone-rod dystrophy, x-linked, type 1 = cAc; secondary glaucoma = cad; macular degeneration,
x-linked atrophyic = cAd; stomatognathic system abnormalities = cAD; neural tube defect, folate-sensitive = caD; 1p(a)
deficiency, congenital = cae; myopia 23, autosomal recessive = caE; metaphyseal dysplasia with maxillary hypoplasia and
brachydactyly = cAE; cone-rod dystrophy 13 = cAe; leber congenital amaurosis 6 = cAf; recurrent abdominal pain = caF;
hypotrichosis, localized, autosomal recessive, 3 = caf; cleidocranial dysplasia, forme fruste, with brachydactyly = cAF;
woolly hair, autosomal recessive 1, with or without hypotrichosis = cag; leigh syndrome , french canadian type = caG;
lack of bone formation = cAG; meckel syndrome, type 5 = cAg; joubert syndrome 7 = cAh; ciliary dyskinesia, primary, 19 =
caH; testicular yolk sac tumor = cAH; myoglobinuria, acute recurrent, autosomal recessive = cah; malignant hyperthermia
susceptibility type 1 = cAI; sapho syndrome = cai; ribose 5-phosphate isomerase deficiency = cAi; agammaglobulinemia
5, autosomal dominant = caI; autism, susceptibility to, x-linked 5 = cAj; type i hyperlipidaemia = caj; king denborough
syndrome = cAJ; parkinson disease 8, autosomal dominant = caJ; idiopathyic disease = caK; ectopic liver = cak; diamond-blackfan
anemia 12 = cAk; neuromuscular disease, congenital, with uniform type 1 fiber = cAK; genital herpe = cAl; charcot-marie-tooth
disease, axonal, type 2p = caL; familial lipoprotein deficiency = cal; fetal akinesia = cAL; secondary hypertriglyceridemia =
cam; ventricular tachycardia, catecholaminergic polymorphic, 2 = cAM; deafness, autosomal recessive 63 = caM; brucellosi =
cAm; diamond-blackfan anemia 9 = cAn; jacobs syndrome = can; arrhythmogenic right ventricular dysplasia, familial,
2 = cAN; microcephalyic osteodysplastic primordial dwarfism, type 1 = caN; leber congenital amaurosis 14 = cao; leprosy,
early-onset, susceptibility to = caO; anemia, diamond-blackfan, 3 = cAo; caffeine dependencye = cAO; wounds, nonpenetrating =
caP; effort syncope = cAP; retinitis punctata albescens (disorder) = cap; mental retardation, x-linked 19 = cAp; bidirectional
tachycardia = cAQ; retinal dystrophy, early-onset severe, Irat-related = caq; tetraplegiac cerebral palsy = caQ; arrhythmogenic
right ventricular dysplasia, familial, 5 = cAq; meibomian gland dysfunction = cAR; immunodeficiency, common variable,
8, with autoimmunity = car; mitochondrial dna depletion syndrome 8a (encephalyomyopathyic type with renal tubulopathy) =
cAr; byssinosis = caR; progressive external ophthalmoplegia with mitochondrial dna deletions, autosomal dominant, 5 =
cAs; night blindness, congenital stationary, type 1f = cas; parachordoma = cAS; fish diseases = caS; thoracic aorta
dilation = cAT; omphalocele, autosomal = cat; infection with mycobacterium marinum = caT; mitochondrial dna depletion
syndrome 8b (mngie type) = cAt; oedema auricular = caU; retinitis pigmentosa 47 = cAU; malignant tumor of optic nerve =
cau; primary retinal cyst = cAu; donnai-barrow syndrome = cav; glaucoma 3, primary congenital, d = caV; ciliary dyskinesia,
primary, 24 = cAv; townes-brocks-branchiootorenal-like syndrome = cAV; duane-radial ray syndrome = cAW; worth syndrome =
caw; weill-marchesani syndrome 3 = caW; ciliary dyskinesia, primary, 11 = cAw; ciliary dyskinesia, primary, 12 = cAx;
heart failure with reduced ejection fraction = caX; osteopetrosis autosomal dominant type 1 = cax; oculootoradial syndrome =
cAX; tumoral calcinosis, normophosphatemiac, familial = cAY; exudative vitreoretinopathy 4 = cay; tooth agenisi, selective,
6 = caY; nail disorder, nonsyndromeic congenital, 4 = cAy; aicardi-goutieres syndrome 5 = cAZ; cutis laxa with severe
pulmonary, gastrointestinal, and urinary abnormalities = caZ; van buchem disease type 2 = caz; dyskeratosis congenita,
autosomal recessive, 5 = cAz; vascular malformations = Cb; leukodystrophy = cb; status epilepticus = cB; human immunodefiency
virus encephalyitis = CB; chilblain lupus 2 = cBa; beta-mannosidosis = cbA; epileptic encephalyopathy, early infantile,
11 = cBA; pulmonary disease, chronic obstructive, severe early-onset = cba; seizures, benign familial infantile, 3 =
cBB; brunner syndrome = cbB; aspirin sensitivity = cbb; sarcosinemia = cBb; antisocial behavior following childhood maltreatment,
susceptibility to = cbC; unspecified circulatory system disorder = cbc; hyperuricemia, pulmonary hypertension, renal
failure, and alkalosis = cBc; epilepsy, benign neonatal, 3 = cBC; gestational diabete = cbd; late onset epilepsy = cBD;
adult antisocial behavior = cbD; cleft palate, isolated, and mental retardation = cBd; rituals = cbE; chromosome 2q32-q33
deletion syndrome = cBe; febrile seizures associated with afebrile seizures = cBE; combined oxidative phosphorylation
deficiency 19 = cbe; atrial fibrillation, familial, 14 = cBF; cleft palate, isolated = cBf; chronic abdominal pain =
cbf; atypical depressive disorder = cbF; hypothyroidism = cbG; corpus callosum, agenisi of, with facial anomalies and
robin sequence = cBg; epilepsy in children = cBG; schwannomatosis 2 = cbg; neuropil threads = cbH; charcot-marie-tooth
disease, type 4b3 = cBh; charcot-marie-tooth disease, type 2a = cbh; brugada syndrome 7 = cBH; charcot-marie-tooth neuropathy,
type 4b2, with early-onset glaucoma = cBi; cardiofaciocutaneous syndrome 3 = cbI; adhesion of intestine = cbi; hypokalemic
periodic paralysis, type 2 = cBI; cataract, pulverulent, juvenile-onset = cbj; cardiofaciocutaneous syndrome 4 = cbJ;
myasthenic syndrome, congenital, acetazolamide-responsive = cBJ; high density lipoprotein cholesterol level quantitative
trait locus 6 = cBj; marden walker like syndrome = cBk; normokalemic periodic paralysis, potassium-sensitive = cBK; osteolysis,
hereditary, of carpal bones with or without nephropathy = cbk; 46,xy sex reversal 6 = cbK; central nervous system demyelination =
cbl; long qt syndrome 10 = cBL; dermatophilosis due to dermatophilus congolensis = cbL; polyposis syndrome, hereditary
mixed, 1 = cBl; cat allergy = cBm; long qt syndrome 3 = cBM; hepatoma, morris = cbM; nerve compression syndrome = cbm;
episodic pain syndrome, familial, 2 = cBn; pulsus trigeminus = cbN; appendiciti = cbn; sick sinus syndrome 1, autosomal
recessive = cBN; neuropathy, hereditary sensory and autonomic, type vii = cBo; prader-willi-like syndrome = cbo; cardiomyopathy,
dilated, 1e = cBO; urethral obstruction = cbO; mental retardation, x-linked 95 = cbp; epileptic encephalyopathy, lennox-gastaut
type = cbP; episodic pain syndrome, familial, 3 = cBp; long qt syndrome type 3 = cBP; ventricular fibrillation, paroxysmal
familial, 1 = cBQ; immunodeficiency, x-linked, with magnesium defect, epstein-barr virus infection, and neoplasmia =
cbq; precapillary pulmonary hypertension = cbQ; migraine, familial hemiplegiac, 3 = cBq; retinitis pigmentosa 62 = cbr;
febrile convulsions, familial, 3a = cBr; atrial fibrillation, familial, 10 = cBR; familial encephalyopathy with neuroserpin
inclusion bodies = cbR; immunodeficiency 12 = cbs; myoclonic astatic epilepsy = cBs; progressive supranuclear palsy atypical =
cbS; heart block, nonprogressive = cBS; benign occipital epilepsy of childhood - early onset variant = cBt; thymus
mucoepidermaoid carcinoma = cbt; supranuclear paralysis = cbT; cardiac conduction defect, nonprogressive = cBT; hemispatial
neglect = cbU; thymus adenosquamous carcinoma = cbu; poor short-term memory = cBu; long qt syndrome 3, acquired, susceptibility
to (finding) = cBU; hypospadias 2, x-linked = cbv; gaze palsy = cbV; lenegre's disease = cBV; grand mal status epilepticus =
cBv; scrotal hypospadias = cbw; sudden unexpected nocturnal death syndrome = cBW; symptomatic generalized epilepsy =
cBw; progressive subcortical gliosis = cbW; familial sick sinus syndrome = cBX; mental retardation, autosomal recessive
15 = cbx; brugada syndrome 5 = cBx; familial progressive supranuclear palsy = cbX; infantile liver failure syndrome 2 =
cbY; bradyarrhythmia = cBY; alpha-mannosidosis = cby; atrial fibrillation, familial, 13 = cBy; sick sinus = cBZ; cardiac
conduction defect, nonspecific = cBz; ataxia, spastic, 3, autosomal recessive = cbZ; lysosomal beta-mannosidase deficiency =
cbz; infantile epileptic encephalyopathy = cC; cardiomyopathy = cc; combined immunodeficiency disease = CC; brain abscess =
Cc; deafness, autosomal recessive 49 = cca; thyroid hormone metabolism, abnormal = cCA; neurally-mediated syncope =
cCa; olmsted syndrome = ccA; ichthyosis follicularis atrichia photophobia syndrome = ccB; epiphyseal dysplasia, multiple,
5 = ccb; diabetic maculopathy = cCB; epileptic encephalyopathy, early infantile, 13 = cCb; shwartzman phenomenon = cCC;
cognitive impairment with or without cerebellar ataxia = cCc; spondyloepimetaphyseal dysplasia, matrilin-3 related =
ccc; palmoplantar keratoderma, mutilating, with periorificial keratotic plaques, x-linked = ccC; burns, electric = cCD;
amyotrophyic lateral sclerosis 21 = ccd; ifap syndrome with or without bresheck syndrome = ccD; indifference to pain,
congenital, autosomal recessive = cCd; myopathy, distal 2 = cce; ichthyosis follicularis with alopecia and photophobia
(ifap) = ccE; erythermalgia, primary = cCe; hypogonadotropic hypogonadism 16 with or without anosmia = cCE; brain anomalies,
retardation, ectodermal dysplasia, skeletal malformations, hirschsprung disease, ear/eye anomalies, cleft pa . . . =
ccF; brachial plexus lesion = cCF; infection due to vancomycin intermediate staphylococcus aureus = ccf; generalized
epilepsy with febrile seizures plus, 7 = cCf; keloidalis nuchae = ccG; infection due to vancomycin resistant staphylococcus
aureus = ccg; febrile seizures, familial, 3b = cCg; brachial plexus lesio = cCG; folliculitis = ccH; infections, bunyavirus =
cch; retinitis pigmentosa 35 = cCH; bronchiectasis with or without elevated sweat chloride 2 = cCh; cone-rod dystrophy
10 = cCI; melanoma, cutaneous malignant, susceptibility to, 5 = ccI; bronchiectasis with or without elevated sweat chloride
3 = cCi; crush syndrome = cci; vaginal yeast = ccj; melanoma, cutaneous malignant, susceptibility to, 1 = ccJ; chronic
cerebrovascular accident = cCJ; immunoglobulin g3 deficiency = cCj; increased analgesia from kappa-opioid receptor agonist,
female-specific = ccK; type vi ehlers-danlos syndrome = cCK; mental retardation, autosomal dominant 1 = cck; infantile
encephalyopathy and lactic acidosis = cCk; uv-induced skin damage, susceptibility to = ccL; atypical pneumonia = ccl;
immunoglobulin g4 deficiency = cCl; histiocytosis = cCL; blood group, john milton hagen system = cCM; secondary immune
deficiency = ccm; cardioencephalyomyopathy, fatal infantile, due to cytochrome c oxidase deficiency = cCm; protein-losing
enteropathy = ccM; body mass index quantitative trait locus 9 = ccN; dengue hemorrhagic feve = cCN; myopia 6 = cCn; tuberculosis
in children = con; hidradenitis = ccO; amyotrophyic neuralgia = cCO; bronchopulmonary disease = cco; senior-loken syndrome
7 = cCo; mitochondrial complex ii deficiency = cCp; septicemia due to streptococcus pneumoniae = ccp; laryngitis = ccP;
brachial plexus neuropathy = cCP; 3-methylcrotonyl coa carboxylase 2 deficiency = ccQ; brachial plexus neuritis = cCQ;
paragangliomas 5 = cCq; arthritis of hand = ccq; cardiomyopathy, dilated, 1gg = cCr; cranial arteritis = ccr; spermatogenic
failure 10 = cCR; propionic acidemi = ccR; pontocerebellar hypoplasia, type 2d = cCS; leigh syndrome due to mitochondrial
complex ii deficiency = cCs; postoperative myocardial infarction = ccs; methylmalonyl-coa epimerase deficiency = ccS;
otitis media in children = cct; succinate-coenzyme q reductase deficiency = cCt; indifference to pain = ccT; 3-methylglutaconic
aciduria with deafness, encephalyopathy, and leigh-like syndrome = cCT; factor v and factor viii, combined deficiency
of, 2 = ccU; neonatal pneumonia = ccu; paragangliomas 2 = cCu; thiamine deficiency = cCU; natural killer cell deficiency,
familial isolated = ccV; fungal pneumonia = ccv; paragangliomas 3 = cCv; panacinar emphysema = cCV; chromosome 18, trisomy
18q = ccw; autosomal hereditary disorder = cCW; cowden syndrome 3 = cCw; microcephaly, primary autosomal recessive, 1 =
ccW; paragangliomas with sensorineural hearing loss = cCx; chronic disease of respiratory system = cCX; chromosome
18q syndrome = ccx; mucocutaneous ulcer = ccX; accelerated tumor formation, susceptibility to = ccY; craniolenticulosutural
dysplasia = cCy; obstructive emphysema = cCY; vacuolar myelopathy = ccy; nodular nonsuppurative panniculitis = cCZ; hemorrhagic
fever, american = ccz; pneumocystosi = cCz; retractile mesenteritis = ccZ; congenital vascular anomaly = Cd; reflex epilepsy =
cD; congestive heart failure = cd; pediatric disease or disorder = CD; amyotrophyic lateral sclerosis 4, juvenile =
cDA; pancreatoblastoma = cDa; abdomen, acute = cdA; connective tissue cancer = cda; spindle cell lipoma = cdb; polyserosistis =
cdB; cholinergic urticaria = cDb; transitional meningioma = cDB; malignant fibrous histiocytoma of bone = cdc; recurrent
fevers = cdC; pulmonary alveolar proteinosis, congenital = cDC; corticosteroid-binding globulin deficiency = cDc; inherited
thyroxine-binding globulin deficiency = cDd; acute suppurative arthritis = cdD; surfactant metabolism dysfunction, pulmonary,
2 = cDD; adult medulloblastoma = cdd; thyroxine-binding globulin deficiency = cDe; combined oxidative phosphorylation
deficiency 18 = cDE; unilateral retinoblastoma = cde; myopathy, areflexia, respiratory distress, and dysphagia, early-onset =
cdE; muscular dystrophy, limb-girdle, type 2d = cDF; deafness, autosomal recessive 91 = cDf; spindle cell liposarcoma =
cdf; carpenter syndrome 2 = cdF; mental retardation, x-linked, syndromeic 13 = cdg; limb-girdle muscular dystrophy,
type 2e = cDG; pituitary macroadenoma = cdG; palmoplantar keratoderma, nagashima type = cDg; antithrombin iii deficiency,
familial = cDh; limb-girdle muscular dystrophy type 2f = cDH; pancreatic gastrinoma = cdH; autism, susceptibility to,
x-linked 3 = cdh; anticoagulant disorders = cDi; cardiomyopathy, dilated, 11 = cDI; klippel feil syndrome recessive type =
cdI; encephalyopathy, neonatal severe, due to mecp2 mutations = cdi; heparin cofactor ii deficiency = cDj; hereditary
protein s deficiency = cdJ; mucopolysaccharidosis, type iiia = cDJ; rett syndrome, zappella variant = cdj; plasminogen
activator inhibitor-1 deficiency = cDk; induced ventricular tachycardia = cdk; lupus hepatitis = cDK; eventration = cdK;
giant cell reparative granuloma = cDL; thrombosis of renal veins = cDl; symptomatic human immunodeficiency virus infection =
cdL; lubs syndrome = cdl; fibrinolytic defect = cDm; ter haar syndrome = cDM; rett syndrome, preserved speech variant =
cdm; pendular nystagmus = cdM; borrone di rocco crovato syndrome = cDN; mosaicism, chromosomal = cdN; fibrous nodule =
cDn; ppm-x syndrome = cdn; giardia infections = cdO; myocardial infarction (subendocardial) = cDo; charcot-marie-tooth
disease, type 4c = cDO; lujan fryns syndrome = cdo; osteogenesis imperfecta, type xii = cDp; cystic lymphangioma = cdP;
ohdo syndrome, maat-kievit-brunner type = cdp; mononeuropathy of the median nerve, mild = cDP; anti-plasmin deficiency,
congenital = cDq; retinitis pigmentosa 38 = cdQ; inherited optic neuropathy = cdq; autism, susceptibility to, 17 = cDQ;
legal blindness = cdr; severe expressive language delay = cDR; dysostosi = cdR; alpha-2-antiplasmin deficiency = cDr;
deaf-blind disorders = cds; autism, susceptibility to, 9 = cdS; 22q deletion syndrome = cDS; familial hemorrhagic diathesis =
cDs; congenital naevus = cdT; diffuse peritoneal leiomyomatosis = cdt; holoprosencephaly 3 = cDT; angioedema, hereditary,
type i = cDt; microcephaly, postnatal progressive, with seizures and brain atrophy = cdu; microphthalmia, isolated, with
coloboma 5 = cDU; malignant fibroxanthom = cdU; complement component 4, partial deficiency of = cDu; hereditary angioedema
type ii = cDv; melanotic neuroectodermal tumor = cdV; mental retardation, autosomal recessive 18 = cdv; absence of tibia =
cDV; epithelial ingrowth = cDW; charcot-marie-tooth disease, type 2b2 = cdw; nanophthalmos 2 = cdW; osteogenesis imperfecta,
type x = cDw; ectopic gastric tissue = cDX; microphthalmia, posterior, with retinitis pigmentosa, foveoschisis, and optic
disc drusen = cdX; pancreatic insufficiency, combined exocrine = cDx; coronary artery disease, autosomal dominant, 1 =
cdx; noonan syndrome-like disorder with loose anagen hair = cDY; emphysema or copd = cDy; optic disk drusen = cdY;
mental retardation, autosomal dominant 20 = cdy; mental retardation, autosomal dominant 23 = cDz; familial mediaterranean
fever, autosomal dominant = cdz; hyperopi = cdZ; noonan-like syndrome with loose anagen hair = cDZ; gastroesophageal
reflux disease = cE; saccharopinuria = ce; secondary acquired sideroblastic anemia = CE; pulmonary involvement = Ce;
short stature, idiopathyic, x-linked = cEa; ceroid lipofuscinosis, neuronal, 7 = cea; erythrocyte lactate transporter
defect = cEA; cecum carcinoma = ceA; hyperinsulinemiac hypoglycemia, familial, 7 = cEB; stocco dos santos syndrome =
cEb; congenital disorder of glycosylation type 2a = ceb; colorectal cancer, hereditary nonpolyposis, type 7 = ceB; low
t3 syndrome = cEC; allergic colitis = ceC; disaccharidase deficiency = cEc; pleomorphic hyalinizing angiectatic tumor =
cec; mitochondrial dna depletion syndrome 11 = ced; acute myeloid leukemia, llq23 abnormalities = ceD; autoimmune disease,
susceptibility to, 6 = cEd; cataract, juvenile, with microcornea and glucosuria = cED; copd exacerbation = cEe; renal
glycosuri = cEE; keutel syndrome = cee; griscelli syndrome type 3 = ceE; singleton merten syndrome = cef; amyotrophyic
lateral sclerosis 16, juvenile = cEf; x-linked hypophosphatemiac ricket = cEF; methylmalonic aciduria cbla type = ceF;
warm autoimmune hemolytic anemia = cEg; glycogen storage disease ic = cEG; adenosylcobalamin synthesis defect = ceG;
vitamin k deficiency coagulation disorder = ceg; disorder of vitamin b12 = ceH; uric acid concentration, serum, quantitative
trait locus 4 = cEH; left ventricular noncompaction 7 = ceh; binge drinking = cEh; tendinitis = cEi; refractory periodontitis =
ceI; endocervical adenocarcinoma in situ = cei; infantile sialic acid storage disease = cEI; methylmalonic aciduria
and homocystinuria, cold type = ceJ; pellagra = cEj; free sialic acid storage disease = cEJ; atypical cartilaginous tumor =
cej; branchiootic syndrome 3 = cEk; homocystinuria, cold type, variant 1 = ceK; deafness, autosomal dominant 25 = cEK;
conjunctiva squamous cell carcinoma = cek; thiamine responsive megaloblastic anemia syndrome = cEL; methylmalonic aciduria,
cbld type, variant 2 = ceL; deafness, autosomal dominant 23 = cEl; myopathy with extrapyramidal signs = cel; branchiootorenal
syndrome 2 = cEm; congenital cleft larynx and opitz-frias syndrome = cem; miyoshi muscular dystrophy 2 = ceM; thiamine-responsive
megaloblastic anemia = cEM; basal ganglia disease, biotin-responsive = cEN; malignant lymphoma - centrocytic = ceN; cataract
15, multiple types = cen; microphthalmia, isolated, with cataract 2 = cEn; microcystic stromal tumor = ceO; dicarboxylicaminoaciduria =
cEO; legionellosis = ceo; acid base disorder = cEo; superior limbic keratoconjunctivitis = ceP; schizophrenia 18 =
cEP; tietz syndrome = cep; paraquat lung = cEp; progressing stroke = cEQ; melanoma, cutaneous malignant, susceptibility
to, 8 = ceq; decreased drug resistance = ceQ; trichohepatoenteric syndrome 2 = cEq; astrocytoma of brain = ceR; bile
acid malabsorption, primary = cEr; episodic ataxia, type 6 = cER; albinism ocular late onset sensorineural deafness =
cer; renal hypouricemia = cES; albinism, ocular, with sensorineural deafness = ces; conjunctivochalasis = ceS; mycobacterium
tuberculosis, susceptibility to infection by = cEs; rhabdomyosarcoma 1 = cET; buruli ulcer, susceptibility to = cEt;
spondyloepimetaphyseal dysplasia, missouri type = ceT; crest syndrome = cet; inflammatory bowel disease 5 = cEU; metaphyseal
anadysplasia = ceU; cutaneous leishmaniasis, ethiopian = cEu; bardet-biedl syndrome 6 = ceu; systemic primary carnitine
deficiency disease = cEV; anemia, hypochromic microcytic, with iron overload = cEv; recurrent malignant peripheral nerve
sheath tumor = ceV; hydrometrocolpos = cev; night blindness, congenital stationary, type 1d = cEW; winchester syndrome =
ceW; precocious puberty, central, 2 = cew; anemia, hypochromic microcytic, with iron overload 1 = cEw; kaler garrity
stern syndrome = ceX; variola major = cex; skin/hair/eye pigmentation, variation in, 6 = cEX; bartter syndrome, antenatal
type 1 = cEx; bardet-biedl syndrome 13 = cey; skin/hair/eye pigmentation, variation in, 4 = cEY; bronchial neoplasm =
ceY; concealed vomiting = cEy; dystonias, sporadic = cEz; sebaceous glands--tumors = cez; hypomyelination, global cerebral =
cEZ; osteolysis hereditary multicentric = ceZ; angiodysplasia of colon = Cf; narcolepsy = cF; combined t cell and b
cell immunodeficiency = CF; hyperlysinemia, type i = cf; autosomal recessive hypophosphatemiac bone disease = cFA; neonatal-onset
citrullinemia type 2 = cFa; acute ischemiac colitis = cfa; congenital disorder of glycosylation, type iib = cfA; aminoacidemia =
cFb; valvular disease = cfb; fanconi renotubular syndrome 2 = cFB; mitochondrial pyruvate carrier deficiency = cfB;
idiopathyic multicentric osteolysis = cfc; congenital disorder of glycosylation type 1f = cfC; amish lethal microcephaly =
cFc; potassium deficiency = cFC; hydrocephalyus, nonsyndromeic, autosomal recessive 2 = cfD; microlithiasis = CFD;
mycetoma = cfd; thiamine metabolism dysfunction syndrome 4 (bilateral striatal degeneration and progressive polyneuropathy
type) = cFd; congenital disorder of glycosylation type 1b = cfE; hypophosphatemiac rickets with hypercalciuria, hereditary =
cFE; wrinkle = cfe; mitochondrial phosphate carrier deficiency = cFe; medullary nephrocalcinosis = cFF; progressive
external ophthalmoplegia with mitochondrial dna deletions, autosomal dominant, 2 = cFf; parotitis = cfF; recurrent pterygium =
cff; deficiency of mannose-6-phosphate isomerase = cfG; clear cell meningioma = cfg; mitochondrial dna depletion syndrome
12 (cardiomyopathyic type) = cFg; congenital disorder of glycosylation, type iif = cFG; atelosteogenesis type 2 = cfh;
amelogenesis imperfecta, hypomaturation type, iia2 = cfh; impaired left ventricular function = cfH; congenital disorder
of glycosylation, type iim = cFH; arthrogryposis, mental retardation, and seizures = cFI; epiphyseal dysplasia, multiple,
4 = cFi; coronary heart disease, susceptibility to, 6 = cfi; mirror hands = cfI; achondrogenesis type 1b = cFj; interdigitating
dendritic cell sarcoma = cFJ; mercaptolactate-cysteine disulfiduria = cfJ; aortic root dilation = cfj; de la chapelle
dysplasia = cFk; navajo neurohepatopathy = cfK; iminoglycinuria = cFK; disorder of tendon = cfk; spermatocele = cFl;
cochlear disease = cfL; glycinuria with or without oxalate urolithiasis = cFL; gastrointestinal ulcer = cfl; cerebral
arterial disease = cfm; tick-borne relapsing feve = cFM; deafness, autosomal recessive 61 = cFm; charcot-marie-tooth
disease, type 2j = cfM; cystic fibrosis, modifier of, 1 = cFn; internal carotid artery stenosis = cfn; foveal hypoplasia
2 = cFN; charcot-marie-tooth disease, dominant intermediate d = cfN; mucus cast = cFo; metaphyseal anadysplasia 2 = cfo;
foveal hypoplasia and anterior segment dysgenesis = cFO; peripheral demyelinating neuropathy, central dysmyelination,
waardenburg syndrome, and hirschsprung disease = cfO; respirovirus infections = cfp; hereditary areflexic dystasisa =
cfP; spondylocheirodysplasia, ehlers-danlos syndrome-like = cFP; intestinal obstructio = cfp; anterior subcapsular cataract =
cfq; ichthyosis prematurity syndrome = cFq; tonic pupil = cfQ; inherited aminoaciduria = cFQ; hereditary stomatocytosis =
cfR; chromosome 12p deletion = cfr; cystinuria, type a = cFR; west nile encephalyitis = cfr; dysosteosclerosis = cFs;
left ventricular hypoplasia = cFS; glucocorticoid deficiency 2 = cfS; rhabdoid meningioma = cfs; combined oxidative phosphorylation
deficiency 9 = cfT; qualitative platelet disorder = cft; familial renal glucosuria = cFt; atrial enlargement = cFT; hypouricemia,
renal, 2 = cFu; combined oxidative phosphorylation deficiency 16 = cfU; familial meningioma = cfu; primary intraocular
non-hodgkin malignant lymphoma = cFU; hypermanganesemia with dystonia polycythemia and cirrhosis = cFv; oculocutaneous
albinism, type iv = cFV; anorectal anomalies = cfv; combined oxidative phosphorylation deficiency 2 = cfV; skin/hair/eye
pigmentation, variation in, 5 = cFW; acrodermatitis = cFw; combined oxidative phosphorylation deficiency 5 = cfW; meningocele =
cfw; molybdenum cofactor deficiency, complementation group a = cfx; immunodeficiency, common variable, 5 = cfX; spherocytosis,
type 4 = cFX; spastic paraplegia 42, autosomal dominant = cFx; renal tubular acidosis, distal, with hemolytic anemia =
cFY; sebaceous adenoma = cfY; molybdenum cofactor deficiency, complementation group b = cfy; congenital cataracts,
hearing loss, and neurodegeneration = cFy; cerebral primitive neuroectodermal tumor = cfZ; blood group--wright antigen =
cFZ; nephrolithiasis-osteoporosis, hypophosphatemiac, 1 = cFz; narcolepsy 7 = cfz; trypanosomiasis = CG; niemann-pick
disease, type b = cG; dural arteriovenous fistula = Cg; hyperlysinemia = cg; negativism in catatonia = cGA; renal tubular
acidosis, distal, with normal red cell morphology = cGa; colorectal cancer, hereditary nonpolyposis, type 5 = cga; hemifacial
spasm = cgA; physical illness = cGB; prostate cancer, hereditary, 13 = cgb; spinal injuries = cgB; blood group--waldner
type = cGb; blushing = cGC; ck syndrome = cgc; he, stomatocytic = cGc; congenital cardiac malformations = cgC; generalized
social phobia = cGD; congenital anomaly of aortic arch = cgD; hereditary prostate carcinoma = cgd; blood group--froese =
cGd; pseudohyperkalemia cardiff = cGe; deafness, autosomal recessive 74 = cge; bullying = cGE; encephalyomalacia =
cgE; performance anxiety = cGF; lateral sinus thrombosis = cgF; lipom = cgf; corneal dystrophy, fuchs endothelial, 4 =
cGf; discrete subaortic stenosis = cgG; corneal endothelial dystrophy type 2 = cGg; myostatin-related muscle hypertrophy =
cgg; hyperekplexia 3 = cGG; myelomeningocele = cgH; brain edem = cGH; corneal endothelial dystrophy 1, autosomal dominant =
cGh; orofacial cleft 5 = cgh; retinitis pigmentosa 68 = cGI; congenital hereditary endothelial dystrophy = cGi; familial
hypodontia = cgi; charcot-marie-tooth disease, type 4b1 = cgI; corneal edema = cGj; charcot-marie-tooth disease, type
4b2 = cgJ; ostium secundum atrial septal defect = cgj; immune deficiency disease = cGJ; unilateral cleft lip = cgk; congenital
muscle disorder = cgK; chronic idiopathyic thrombocytopeniac purpura = cGK; renal tubular acidosis, proximal, with ocular
abnormalities and mental retardation = cGk; tooth and nail syndrome = cgl; bilateral glaucoma = cGl; combined oxidative
phosphorylation deficiency 10 = cgL; lysinuric protein intolerance = cGL; craniosynostosis, type 2 = cgm; recombinant
chromosome & syndrome = cGm; spastic ataxia 4, autosomal recessive = cgM; cystinuria, type b = cGM; nephrolithiasis-osteoporosis,
hypophosphatemiac, 2 = cGN; homocystinuria-megaloblastic anemia due to defect in cobalamin metabolism, cble complementation
type = cgN; parietal foramina with cleidocranial dysplasia = cgn; brown-vialetto-van laere syndrome = cGn; brown-vialetto-van
laere syndrome 2 = cGo; parietal foramina 1 = cgo; biliary carcinoma = cGO; abdominal obesity metabolic syndrome = cgO;
cleidocranial dysplasia = cgp; medullary cystic kidney disease type 1 = cgP; progressive bulbar palsy of childhood =
cGp; episodic kinesigenic dyskinesia 2 = cGP; atopic keratoconjunctivitis = cgQ; progressive bulbar palsy = cGq; mental
retardation, x-linked, syndromeic, christianson type = cGQ; activated pi3k-delta syndrome = cgq; lymphoblastic leukemia,
acute, with lymphomatous features = cgr; christianson syndrome = cGR; thyroid dyshormonogenesis 1 = cGr; ileoanal reservoir =
cgR; fibrosis of pleura = cgs; cestode infections = cgS; neuropathy, distal hereditary motor, type viia = cGs; gangrenous
pneumonia = cGS; combined oxidative phosphorylation deficiency 15 = cgt; bile duct cystadenocarcinoma = cgT; panic symptoms =
cGt; abscess of breast = cGT; intestinal nematode infection = cgU; body mass index quantitative trait locus 11 = cGu;
neonatal diarrhea = cgu; healtyhcare associated pneumonia = cGU; arthralgia of temporomandibular joint = cGv; methylcobalamin
deficiency, cblg type = cgv; myasthenia gravis, generalized = cgV; ischemiac foot = cGV; homocystinuria due to deficiency
of n(5,10)-methylenetetrahydrofolate reductase activity = cgw; adverse reaction to drug = cGw; diabetic foot infection =
cGW; methylmalonic aciduria due to methylmalonyl-coa mutase deficiency = cgW; otitis externa = cGX; loneliness in adolescence =
cGx; methylenetetrahydrofolate reductase deficiency = cgx; methylmalonic aciduria, mut(—) type = cgX; mediastiniti =
cGY; methylmalonic aciduria, mut(0) type = cgY; depression in children = cGy; thrombosis of retinal veins = cgy; ritualistic
behavior (symptom) = cGz; colorectal adenomatous polyposis, autosomal recessive = cgZ; fetus affected by placental transfer
of anticonvulsant = cgz; autism, susceptibility to, 16 = cGZ; squamous cell carcinoma of pharynx = ch; pleural tuberculosis =
CH; hypercholesterolemia = cH; thromboxane synthetase deficiency = Ch; hyper-igd periodic fever syndrome (hids) = cha;
hearing disability = chA; b-cell immunodeficiency, distal limb anomalies, and urogenital malformations = cHA; rotor syndrome =
cHa; anophthalmos with limb anomalies = cHB; hypertrophyic osteoarthropathy, primary, autosomal recessive, 2 = cHb;
inclusion body myopathy 3, autosomal dominant = chB; african swine fever = chb; epstein syndrome = chC; polymorphous
low grade adenocarcinoma = chc; niemann-pick disease, intermediate, protracted neurovisceral = cHC; deafness, cochlear,
with myopia and intellectual impairment = cHc; arthrogryposis, distal, type 1b = chd; deafness, x-linked 4 = cHD; inclusion
body myopathy, autosomal dominant = chD; peri-menopausal = cHd; snyder robinson syndrome = cHE; distal arthrogryposis
type 2b = chE; lethal congenital contracture syndrome 4 = che; loeys-dietz syndrome 3 = che; arthrogryposis multiplex
congenita, distal type 1 = chF; waardenburg syndrome, type 2d = cHF; juvenile polyposis with hereditary hemorrhagic telangiectasisa =
cHf; cardiomyopathy, familial hypertrophyic, 4 = chf; left ventricular noncompaction 10 = chg; growth mental deficiency
syndrome of myhre = cHg; retrognathia = chG; neuromuscular manifestations = cHG; cardiomyopathy, familial hypertrophyic,
4, susceptibility to = chh; cardiomyopathy, familial hypertrophyic, 14 = chH; cerebral dysgenesis = cHH; colorectal cancer,
susceptibility to, 3 = cHh; primary hypertrophyic cardiomyopathy = chi; parkinson disease 1, autosomal dominant = cHI;
pulmonary hypertension, primary, 2 = cHi; cardiomyopathy, dilated, lee = chI; sick sinus syndrome 3, susceptibility to =
chJ; spinal meningioma = cHj; parkinson disease 4, autosomal dominant lewy body = cHJ; metastatic pheochromocytoma =
chj; progonoma = chk; atrial septal defect 3 = chK; lameness, animal = cHK; impaired social interaction = cHk; inflammatory
carcinoma = chl; rhabdoid tumor predisposition syndrome 2 = cHl; cocaine delirium = CHL; cardiomyopathy, dilated, 1s =
chL; left ventricular noncompaction 5 = chM; psychomotor retardation, epilepsy, and craniofacial dysmorphism = cHM;
mental retardation, autosomal dominant 16 = cHm; lymphomas in children = chm; rhabdoid tumor predisposition syndrome =
cHn; retinitis pigmentosa 33 = cHN; ebstein's anomaly = chN; medullomyoblastoma = chn; hecht syndrome = chO; hypotrichosis
11 = cHO; mature b-cell neoplasm = cho; rhabdoid tumor predisposition syndrome 1 = cHo; mental retardation, autosomal
dominant 15 = cHp; carney complex variant = chP; neuroblastoma, susceptibility to = chp; mediastinum teratoma = cHP;
macrothrombocytopenia progressive deafness = chQ; chromosome 1, monosomy 1p = chq; long qt syndrome 12 = cHQ; malignant
rhabdoid tumor, somatic = cHq; central neurocytoma = chr; teratoid tumor, atypical = cHr; osteopetrosis, autosomal recessive
8 = cHR; fechtner syndrome = chR; ependymoblastoma = cHs; intermixed schwannian stroma-rich ganglioneuroblastoma = chs;
may-hegglin anomaly = chS; microcephaly, microphthalmia, ectrodactyly of lower limbs, and prognathism = cHS; neurological
ventriculitis = cht; deafness, autosomal dominant 17 = chT; mental retardation, anterior maxillary protrusion, and strabismus =
cHT; brain ependymoma = cHt; deafness, autosomal dominant nonsyndromeic sensorineural 17 = chU; epithelioid malignant
peripheral nerve sheath tumor = cHu; myopathy, centronuclear, 3 = chu; dermatitis, atopic, 4 = cHU; acute nephritis =
cHV; cochleosaccular degeneration of the inner ear and progressive cataracts = chV; familial hyperlipidemi = cHv; aortic
aneurysm, familial thoracic 4 = chv; cornealia de lange syndrome 3 = cHw; clq nephropathy = chW; mitochondrial cytopathy =
cHW; acute myelomaonoblastic leukemia = chw; small intestinal sarcoma = chx; degenerative myelopathy = cHX; facioscapulohumeral
muscular dystrophy 1b = cHx; pathyognomonic sign = chX; adult spinal muscular atrophy = cHy; myopathy, myosin storage =
chy; stiff limbs = cHY; bladder neck obstructio = chY; peripheral neuropathy, myopathy, hoarseness, and hearing loss =
chz; cardiomyopathy, familial hypertrophyic, 10 = chZ; bulbar weakness = cHz; microvascular complications of diabetes,
susceptibility to, 6 = cHZ; hypercholesterolemia, familial = cI; mental condensation = Ci; congenital hemolytic anemia =
CI; carcinoma = ci; chronic pelvic pain syndrome = cIa; acetylation, fast = ciA; cardiomyopathy, familial hypertrophyic,
8 = cia; spastic paraplegia 11, autosomal recessive = cIA; lactic acidosis, fatal infantile = cIb; schindler disease,
type i = ciB; aortic aneurysm, familial thoracic 7 = cib; spastic paraplegia 15, autosomal recessive = cIB; deafness,
autosomal recessive 3 = cic; atrophy of corpus callosum = cIC; kanzaki disease = ciC; noonan syndrome 4 = cIc; neuroaxonal
dystrophy = ciD; deafness, autosomal dominant 48 = cid; craniodiaphyseal dysplasia, autosomal dominant = cId; mast syndrome =
cID; spastic paraplegia 7, autosomal recessive = cIE; n-acetyl glutamate synthetase deficiency = ciE; focal segmental
glomerulosclerosis 6 = cie; osteoarthritis, spine = cIe; hypotonia, infantile, with psychomotor retardation and characteristic
facies = ciF; craniodiaphyseal dysplasia = cIf; acute erythroleukemia = cIF; deafness, autosomal recessive 30 = cif;
diarrhea 3, secretory sodium, congenital, syndromeic = cIG; griscelli syndrome type 1 = cig; spermatogenic failure 12 =
ciG; waardenburg syndrome, type 4c = cIg; yemenite deaf-blind hypopigmentation syndrome = cIh; elejalde disease = cih;
langerhans cell histiocytosis of lung = cIH; anemia due to infection = ciH; diarrhea intractable = cii; tympanosclerosis =
cII; waardenburg syndrome, type 2e = cIi; peripheral osteosarcoma = ciI; parkinson disease 3, autosomal dominant lewy
body = cIJ; deafness, autosomal recessive 37 = cij; flaccid paralysis = ciJ; chronic intestinal pseudo-obstruction =
cIj; neuronal intestinal dysplasia, type b = cIK; deafness, autosomal dominant nonsyndromeic sensorineural 22 = cik;
plasma glucose concentration = ciK; childhood pilocytic astrocytoma = cIk; deafness, sensorineural, with hypertrophyic
cardiomyopathy = cil; hypogonadotropic hypogonadism 17 with or without anosmia = cIL; vesicoureteral reflux 3 = cIl;
sigmoid neoplasm = ciL; hypotrichosis-lymphedema-telangiectasisa syndrome = cIm; deafness, autosomal recessive 2 = cim;
n-acetylaspartate deficiency = ciM; elliptocytosis 2 = cIM; deafness, autosomal dominant 11 = cin; mental retardation,
x-linked, with panhypopituitarism = cIn; short stature, optic nerve atrophy, and pelger-huet anomaly = ciN; spherocytosis,
type 3 = cIN; usher syndrome, type ib = cio; congenital hypertrichosis lanuginosa = cIo; nk/t-cell lymphoma, nasal and
nasal-type = ciO; epileptic encephalyopathy, early infantile, 5 = cIO; spectrin, beta, erythrocytic = cIP; lymph node
tuberculosis = ciP; celiac disease, susceptibility to, 4 = cip; mental retardation, x-linked, with isolated growth hormone
deficiency = cIp; elliptocytosis 3 = cIQ; congenital hypertrichosis = cIq; glaucoma due to combination of mechanisms =
ciq; granulomatous disease, chronic, autosomal recessive, cytochrome b-positive, type i = ciQ; granulomatous disease,
chronic, autosomal recessive, cytochrome b-positive, type ii = ciR; campomelic dysplasia with autosomal sex reversal =
cIr; spinocerebellar ataxia, autosomal recessive 14 = cIR; steroid-induced glaucoma = cir; iga myeloma = cis; neuropathy,
hereditary sensory and autonomic, type ic = cIS; 46,xx sex reversal 2 = cIs; systemic aspergillosis = ciS; streptococcal
impetigo = cit; coloboma, ocular, and ichthyosis, brain malformations, and endocrine abnormalities = cIT; kyphomelic
dysplasia = cIt; granulomatous disease, chronic, autosomal recessive, cytochrome b-positive, type iii = ciT; autoimmune
thyroiditi = ciU; torsion abnormality = cIu; kahrizi syndrome = cIU; myopathy, spheroid body = ciu; bone marrow failure
syndrome 1 = cIV; ciliary dyskinesia, primary, 28 = cIv; posterior lenticonus = ciV; muscular dystrophy, limb-girdle,
type 1a = civ; culture shock = cIw; cardiomyopathy, familial hypertrophyic, 16 = ciw; malaria, mild, susceptibility to =
ciW; rolandic epilepsy, mental retardation, and speech dyspraxia, x-linked = cIW; hidradenitis suppurativa, familial =
ciX; 46,xy gonadal dysgenesis, complete, sry-related = cIX; spastic paraplegia 4, autosomal dominant = cIx; cardiomyopathy,
dilated, 1kk = cix; sclerotylosis = cIY; microdysgenesis = cIy; duration of sleep = ciy; microhydranencephaly = ciY;
lissencephaly 4 = ciZ; ogden syndrome = ciz; oculomaxillofacial dysostosis = cIz; 46, xx true hermaphrodite = cIZ; chronic
lymphocytic leukemia = cj; familial hypoalphalipoproteinemia = cJ; diamond-blackfan anemia = CJ; hereditary hemorrhagic
telangiectasisa = Cj; monophasic synovial sarcoma = cJa; epileptic encephalyopathy, early infantile, 4 = cJA; microlissencephaly =
cja; bone epithelioid hemangioma = cjA; exudative vitreoretinopathy, familial, x-linked recessive = cjb; bronchial
adenocarcinoma = cjB; esophageal varix = cJb; symptomatic west syndrome = cJB; somatostatin analog, resistance to = cJc;
neuropathy, hereditary motor and sensory, lom type = cjc; colorado tick fever = cjC; epileptic spasms = cJC; sotos syndrome
2 = cjD; ichthyosis with hypotrichosis, autosomal recessive = cJd; mitochondrial encephalyopathy, lactic acidosis and
stroke-like episodes (melas syndrome) = cjd; hemophagocytic lymphohistiocytosis, familial, 5 = cJD; thyroid hurthle cell
carcinoma = cje; amish infantile epilepsy syndrome = cJe; mitochondrial dna depletion syndrome 5 (encephalyomyopathyic
with or without methylmalonic aciduria) = cJE; marshall-smith syndrome = cjE; osteochondrodysplasia, rhizomelic, with
callosal agenisi, thrombocytopenia, hydrocephalyus, and hypertension = cjF; mitochondrial dna depletion syndrome 2 (myopathyic
type) = cJF; sarcoidosis, early-onset = cjf; native american myopathy = cJf; sensory polyneuropathy = cjg; benign multiple
sclerosis = cjG; premature ovarian failure 8 = cJg; mitochondrial dna depletion syndrome 9 (encephalyomyopathyic type
with methylmalonic aciduria) = cJG; orthostatic hypotensive disorder, streeten type = cjh; chronic gouty arthritis =
cjH; microcephaly-capillary malformation syndrome = cJh; glutaric aciduria iii = cJH; mycobacterial and viral infections,
susceptibility to, autosomal recessive = cJi; photogenic epilepsy = cji; comedocarcinoma, noninfiltrating = cJI; pediatric
ulcerative colitis = cjI; never in mitosis gene a-related kinase 1 = cjj; candidiasis, familial, 7 = cJj; lyssavirus
infection = cjJ; mammographic breast density = cJJ; subdural empyema = cjK; retinitis pigmentosa 67 = cjk; hyper-ige
recurrent infection syndrome, autosomal dominant = cJk; orofacial cleft 10 = cJK; immunodeficiency, common variable,
10 = cjL; nephronophthisis 9 = cjl; diabetes mellitus, insulin-dependent, 5 = cJL; systemic lupus erythematosus, susceptibility
to, 11 = cJl; renal-hepatic-pancreatic dysplasia 2 = cjm; ectodermal dysplasia, anhidrotic, with t-cell immunodeficiency,
autosomal dominant = cjM; anemia, hypochromic microcytic, with iron overload 2 = cJm; follicular mucinosis = cJM; microcephaly,
primary autosomal recessive, 7 = cJn; renal pelvis transitional cell carcinoma = cjn; pleural empyema = cjN; arrest of
spermatogenesis = cJN; myopathy, tubular aggregate = cJo; epilepsy, x-linked, with variable learning disabilityies and
behavior disorders = cJO; sialidosis, type i = cjo; multiple mitochondrial dysfunctions syndrome 1 = cjO; stormorken
syndrome = cJp; neuropathy, hereditary sensory and autonomic, type v = cjP; mody, type 6 = cjp; emery-dreifuss muscular
dystrophy 4 = cJP; spinocerebellar ataxia, autosomal recessive 8 = cJQ; diabetes mellitus, insulin-dependent, 7 = cjq;
pseudopseudohypoparathyroidis = cJq; deep pain = cjQ; emery-dreifuss muscular dystrophy 5, autosomal dominant = cJR;
diarrhea 4, malabsorptive, congenital = cjr; myofascial pain syndrome = cjR; preeclampsia eclampsia 4 = cJr; polyhydramnios,
megalencephaly, and symptomatic epilepsy = cJs; congenital disorder of deglycosylation = cjS; deafness, autosomal recessive
76 = cJS; neurofibromatosis-noonan syndrome = cjs; epilepsy, progressive myoclonic 2b = cjT; mental retardation, autosomal
dominant 5 = cJT; deafness, autosomal recessive 16 = cJt; neurofibromatosis, familial spinal = cjt; parkinson disease
20, early-onset = cJU; dyskeratosis congenita, autosomal recessive, 1 = cjU; rud syndrome = cJu; caf-au-lait macules
with pulmonary stenosis = cju; mental retardation, x-linked 96 = cJV; dyskeratosis congenita, autosomal recessive = cjV;
retention hyperkeratosis = cJv; neurofibromatosis type 5 = cjv; congenital disorder of glycosylation, type iw = cJw;
learning problems = cjw; spinocerebellar ataxia, autosomal recessive 11 = cJW; dyskeratosis congenita, autosomal recessive,
2 = cjW; congenital disorder of glycosylation, type ix = cJx; cataract, congenital, with microcornea or slight microphthalmia =
cjX; fibrous hamartoma = cjx; sacral agenisi with vertebral anomalies = cJX; seckel syndrome 7 = cjY; myxedema = cJY;
hemophagocytic lymphohistiocytosis, familial, 4 = cly; radiation induced meningioma = cjy; congenital heart defects,
multiple types, 2 = cJZ; spastic paraplegia 6, autosomal dominant = cjZ; tumorlet = cjz; horse diseases = cJz; congenital
hypoplastic anemia = CK; telangiectasis = Ck; leukemia = ck; familial hdl deficiency = cK; chronic cryptosporidiosis =
cKa; ghosal hematodiaphyseal dysplasia = cKA; ichthyosis, congenital, autosomal recessive 6 = cka; glucocorticoid deficiency
4 = ckA; eye manifestations = ckb; cardiomyopathy, dilated, 1n = cKB; hypogonadotropic hypogonadism 10 with or without
anosmia = cKb; premature ovarian failure 5 = ckB; hypogonadotropic hypogonadism 11 with or without anosmia = cKc; muscular
dystrophy, limb-girdle, type 2g = cKC; choreoathetosis, hypothyroidism, and neonatal respiratory distress = ckc; external
auditory canal, bilateral atresia of, with congenital vertical talus = ckC; granulomatous mastitis = ckD; chorea, benign
hereditary = ckd; craniosynostosis 3 = cKD; mental retardation, autosomal recessive 40 = cKd; diabetes mellitus, insulin-dependent,
21 = cKe; corneal dystrophy, fuchs endothelial, 3 = cKE; granulomatous uveitis = ckE; progressive chorea = cke; glomerulonephritis
sparse hair telangiectases = cKf; precursor lymphoblastic lymphoma/leukemia = cKF; neurohypophysis granular cell tumor =
ckf; anus disease = ckF; cranial nerve disease = ckG; transcobalamin ii = cKG; transaldolase deficiency = cKg; hypothyroidism,
congenital, nongoitrous, 5 = ckg; atrial septal defect 7 with or without atrioventricular conduction defects = ckh; transcobalamin
ii deficiency = cKH; situs inversu = ckH; reactive arthritis = cKh; maxillary sinus neoplasm = cKi; nog-related-symphalangism
spectrum disorder = ckI; ventricular septal defect 3 = cki; joubert syndrome 13 = cKI; tectonic family, member 2 = cKJ;
splenic hypoplasia = ckj; thiourea tasting = cKj; symphalangism-brachydactyly syndrome = ckJ; brachydactyly, type b2 =
ckK; hypoplastic left heart syndrome 2 = ckk; joubert syndrome 18 = cKK; hypertyrosinemia = cKk; isolated noncompaction
of the ventricular myocardium = ckl; stapes ankylosis with broad thumb and toes = ckL; forebrain defects = cKL; warburg
micro syndrome 4 = cKl; multiple ventricular septal defects = ckm; epileptic encephalyopathy, early infantile, 16 = cKm;
esophageal atresia with or without tracheoesophageal fistula = ckM; schizophrenia = cKM; deafness, autosomal recessive
86 = cKn; spinocerebellar ataxia, autosomal recessive, with axonal neuropathy = cKN; ostium secundum type atrial septal
defect = ckn; stapes fixation = ckN; carpal synostosis = ckO; ovarian endodermal sinus tumor = cko; digitorenocerebral
syndrome = cKo; cataract 36 = cKO; autism, susceptibility to, x-linked 1 = ckp; sveinsson chorioretinal atrophy = cKP;
kenny caffey syndrome = cKp; myoclonus, familial cortical = ckP; kenny-caffey syndrome, type 1 = cKq; asperger syndrome,
x-linked, susceptibility to, 1 = ckq; spastic paraplegia 49, autosomal recessive = cKQ; achalasia, familial esophageal =
ckQ; atypical autism = ckr; hypoparathyroidism-retardation-dysmorphism syndrome = cKr; mental retardation, autosomal
recessive 14 = cKR; salivary gland basal cell adenocarcinoma = ckR; deafness, autosomal dominant 12 = cKS; asperger syndrome,
x-linked, susceptibility to, 2 = cks; qt interval, variation in = ckS; emanuel syndrome = cKs; deafness, autosomal recessive
21 = cKT; cervical aortic arch = cKt; lymph node infected = ckT; autism, susceptibility to, x-linked 2 = ckt; corneal
intraepithelial dyskeratosis and ectodermal dysplasia = cku; herpes zoste = cKU; facial deformity = cKu; alzheimer disease,
late-onset, susceptibility to = ckU; hypertension in children = ckV; venous malformations, multiple cutaneous and mucosal =
cKV; atrial septal defect 4 = cKv; familial cold autoinflammatory syndrome 2 = ckv; intramuscular hemangioma = cKW;
asthma and nasal polyps = cKw; ventricular failure = ckW; primary gout = ckw; atherosclerotic renal artery stenosis =
ckX; anal canal carcinoma = ckx; abruzzo erickson syndrome = cKx; placenta accreta = cKX; ciliary dyskinesia, primary,
6 = cky; microphthalmia, isolated, with coloboma 9 = cKY; ischiopatellar dysplasia = cKy; cor pulmonale = ckY; congenital
defect of skull and scalp = ckZ; dyskeratosis congenita, autosomal dominant = cKZ; bleeding disorder, platelet-type,
13, susceptibility to = cKz; amaurosis congenita of leber, type 9 = ckz; endometrial carcinoma = cl; angiodysplasia =
Cl; trichomoniasis = CL; arthritis, juvenile = cL; adrenal hypoplasia, congenital, with precocious puberty = clA; valve
anomalies = cla; pulmonary fibrosis and/or bone marrow failure, telomere-related, 1 = cLa; loeys-dietz syndrome 4 = cLA;
dyskeratosis congenita, autosomal dominant, 2 = cLb; alagille syndrome 2 = clb; furlong syndrome = cLB; mineralocorticoid
deficiency, isolated = clB; aneurysm of aortic root = cLC; congenital adrenal hypoplasia, x-linked = clC; serpentine
fibula polycystic kidney syndrome = clc; melanoma, cutaneous malignant, susceptibility to, 9 = cLc; rienhoff syndrome =
cLD; fibromatosis, congenital generalized = cld; primary spermatogenic failure = clD; meningothelial meningioma = cLd;
retinitis pigmentosa 37 = clE; arrhythmogenic right ventricular dysplasia, familial, 1 = cLE; congenital atransferrinemia =
cLe; myofibromatosis, infantile, 2 = cle; corneal dystrophy, lattice type iiia = cLF; goldmann-favre syndrome = clF;
lacune = clf; char syndrome = cLf; binswanger's disease = clg; spastic paraplegia 57, autosomal recessive = cLg; metamorphopsia =
clG; groenouw type i corneal dystrophy = cLG; stromal corneal dystrophy = cLH; neuropathy, hereditary motor and sensory,
okinawa type = cLh; paraneoplasmtic opsoclonus-myoclonus ataxia = clh; macular retinoschisis = clH; cryptosporidiosi =
clI; amyloid of cornea = cLI; corneal guttata = cli; acquired protein s deficiency = cLi; hypochondriasis = clj; corneal
deposit = cLJ; arenaviridae infections = cLj; bosch-boonstra optic atrophy syndrome = clJ; niemann-pick disease, type
a = clk; 46, xx disorders of sex development = clK; corneal granular dystrophy = cLK; howell jolly bodies = cLk; african
hemochromatosis = cLl; reis-bucklers corneal dystrophy = cLL; pseudohermaphroditism, female, with hypokalemia, due to
glucocorticoid resistance = clL; niemann-pick disease, type c1 = cll; thyroid dyshormonogenesis 3 = cLm; thiel-behnke
corneal dystrophy = cLM; niemann-pick disease, type d = clm; microbial colonization = clM; body composition, beneficial =
clN; aortic aneurysm, familial thoracic 3 = cLN; autoimmune thyroid disease, susceptibility to, 3 = cLn; renal-hepatic-pancreatic
dysplasia = cln; nelson syndrome = clO; renal hepatic pancreatic dysplasia dandy walker cyst = cLo; thyroid dysfunction
of mother, postpartum condition or complication = clo; colorectal cancer, hereditary nonpolyposis, type 6 = cLO; pseudohypoaldosteronism,
type i, autosomal dominant = clP; senior-loken syndrome 4 = clp; chronic thyroiditis = cLp; loeys-dietz syndrome, type
1b = cLP; ichthyosis, congenital, autosomal recessive 1 = cLQ; nontoxic goiter = cLq; nephronophthisis 4 = clq; pseudohypoaldosteronism,
type i, autosomal recessive = clQ; hypertension, early-onset, autosomal dominant, with severe exacerbation in pregnancy =
clR; thyroid angiosarcoma = cLr; autosomal recessive ichthyosis = cLR; normal pressure hydrocephalyu = clr; minimal
lesion = cls; keratinization of ocular surface = cLS; estrogen receptor positive tumor = cLs; deficiency of dehydrogenase =
clS; denture stomatitis = clT; dentin dysplasia, type 1 = cLt; asthma-related traits, susceptibility to, 2 = clt; ectropion =
cLT; xanthinuria = cLU; premature ovarian failure 7 = clU; mils = clu; acute mucositis = cLu; ribbing disease = cLv;
peeling skin syndrome, acral type = cLV; bronchioliti = clv; spermatogenic failure 8 = clV; transglutaminase 6 = cLW;
radiation esophagitis = cLw; expressed emotion = clW; after pains = clw; carcinogenesis, radiation = cLx; chronic drug
abuse = clx; laryngeal dystonia = cLX; retinitis pigmentosa 27 = clX; brain ischemiaa = cLy; neural retina leucine zipper =
clY; pancreatic cholera = cly; hemolytic uremic syndrome, atypical, susceptibility to, 6 = cLY; thrombophilia due to
thrombomodulin defect = cLZ; leukemia, post-chemotherapy, susceptibility to = clz; retinal degeneration, autosomal recessive,
clumped pigment type = clZ; epithelioid cell melanoma = cLz; aminoacylase 1 deficiency = Cm; neuroblastoma = cm; lymphopenia =
CM; recurrence = cM; idiopathyic megacolon = cma; leprosy, susceptibility to, 3 = cMA; alpha-ketoglutarate dehydrogenase
deficiency = cmA; laryngeal atresia, encephalyocele, and limb deformities = cMa; schizophrenia 17 = cmb; corneal allograft
rejection = cMb; mixed type cataract = cmB; prosthetic joint infection = cMB; pitt-hopkins-like syndrome 2 = cmc; beaulieu-boycott-innes
syndrome = cMc; sexually transmitted diseases, bacterial = cMC; glaucoma, normal tension, susceptibility to = cmC; osteomyelofibrosis =
cMd; optic atrophy 1 and deafness = cmD; streptococcal sepsis = cMD; verruciform xanthoma of ski = cmd; experimental
pneumococcal meningitis = cMe; optic atrophy with or without deafness, ophthalmoplegia, myopathy, ataxia, and neuropathy =
cmE; rheumatic fever in children = cME; hypogonadotropic hypogonadism 9 with or without anosmia = cme; optic atrophy
and cataract, autosomal dominant = cmF; candida sepsis = cMF; focal thyroiditis = cMf; mental retardation, autosomal
recessive 5 = cmf; 3-methylglutaconic aciduria type iv = cmG; spastic paraplegia 45, autosomal recessive = cmg; supragingival
dental plaque = cMG; hypothyroidism, congenital, nongoitrous, 6 = cMg; 3-methylglutaconic aciduria = cmH; listeria meningitis =
cMH; uridine 5-prime monophosphate hydrolase deficiency, hemolytic anemia due to = cmh; thyroid hormone resistance,
generalized, autosomal dominant = cMh; thyroid hormone resistance, selective pituitary = cMi; herpes simplex encephalyitis,
susceptibility to, 2 = cMI; mental retardation, x-linked, with cerebellar hypoplasia and distinctive facial appearance =
cmI; atrial septal defect 1 = cmi; glaucoma 1, open angle, o = cmj; thyroid hormone resistance, generalized, autosomal
recessive = cMj; blue cone monochromatism = cmJ; macular degeneration, age-related, 10 = cMJ; pain insensitivity, congenital =
cmk; colorblindness, partial, protan series = cmK; tsh producing pituitary tumor = cMk; chronic urinary tract infection =
cMK; acne conglobata = cML; robinow syndrome = cml; herpes simplex encephalyitis, susceptibility to, 4 = cMl; red color
blindness = cmL; dilated cardiomyopathy secondary to viral myocarditis = cMm; tritanopia = cmM; graft ischaemia = cMM;
obesity, hyperphagia, and developmental delay = cmm; osteomyelitis = cMN; jensen syndrome = cMn; oculomotor nerve injuries =
cmn; opioid withdrawal = cmN; necrotizing scleritis = cMo; schizophrenia 3 = cmo; craving for alcohol = cmO; ileiti =
cMO; exercise anaphylaxis = cmp; legionnaire disease, susceptibility to = cMP; secondary disease = cMp; narcotic addiction =
cmP; fundus dystrophy, pseudoinflammatory, of sorsby = cMq; meningitis in children = cMQ; ischemiac pain = cmQ; striatonigral
degeneration infantile = cmq; night blindness, congenital stationary, type la = cmr; revesz debuse syndrome = cMr; neuronal
intestinal pseudoobstruction = cMR; amyotrophyic lateral sclerosis 12 = cmR; glaucoma 1, open angle, e = cmS; ornithine
aminotransferase = cms; dyskeratosis congenita, autosomal dominant, 3 = cMs; specific language impairment 5 = cMS; mycobacterium
tuberculosis, protection against = cMt; infections, arenavirus = cMT; vasomotor rhinitis = cmT; hyperornithinemia = cmt;
toxemia = cmU; atrial septal defect 6 = cMu; deafness, autosomal recessive 7 = cMU; gyrate atrophy = cmu; three m syndrome
2 = cmv; leprosy, susceptibility to, 5 = cMv; severe dehydration = cmV; deafness, autosomal dominant 36 = cMV; autosomal
recessive ocular albinism = cmw; arthrogryposis multiplex congenita neurogenic type = cmW; cerebrofaciothoracic dysplasia =
cMW; antepartum malaria = cMw; optic atrophy 7 = cMX; pain neck/shoulder = cmX; brown oculocutaneous albinism = cmx;
recurrent cystitis = cMx; joubert syndrome 16 = cMY; gram positive sepsis = cMy; joubert syndrome 10 = cmy; c3hex, ability
to smell = cmY; leprosy reversal reaction = cMz; simpson-golabi-behmel syndrome, type 2 = cmz; congenital disorder of
glycosylation, type iik = cMZ; negative rheumatoid factor polyarthritis = cmZ; biotinidase deficiency = Cn; cervical
squamous cell carcinoma = cn; membranoproliferative glomerulonephritis, x-linked = cN; gestational trophyoblastic neoplasm =
CN; joubert syndrome 2 = cNa; childhood type dermatomyositi = cna; myopia, high, with cataract and vitreoretinal degeneration =
cnA; liver scarring = cNA; meckel syndrome type 2 = cNb; segmental ileitis = cNB; dysphagia, progressive = cnB; stomatin-like
protein-2, hyperphosphorylation of = cnb; immune dysfunction with t-cell inactivation due to calcium entry defect 2 =
cnc; ischemiac myocardial dysfunction = cNC; mental retardation, autosomal dominant 17 = cnC; meckel syndrome, type 11 =
cNc; chronic q fever = cND; immune dysfunction with t-cell inactivation due to calcium entry defect 1 = cnd; urate
nephropathy = cnD; joubert syndrome 20 = cNd; coxiella burnetii infection = cNE; endometrioid adenoma = cnE; joubert
syndrome 14 = cNe; meier-gorlin syndrome 2 = cne; severe asthma = cnF; disc disorder = cNF; osteogenesis imperfecta,
type xiv = cNf; meier-gorlin syndrome 3 = cnf; amyloidosis ix = cng; hyperphenylalaninemia, non-pku mild = cnG; emery-dreifuss
muscular dystrophy 7, autosomal dominant = cNg; bunion = cNG; muscular dystrophy-dystroglycanopathy (congenital with
brain and eye anomalies), type a, 10 = cNh; persistent hyperphenylalaninemia = cnH; abdominal abscess = cNH; osteopetrosis,
autosomal recessive 1 = cnh; activity intolerance = cnI; osteopetrosis, autosomal recessive 5 = cni; induration of breast =
cNI; joubert syndrome 6 = cNi; nephronophthisis 11 = cNj; meningoencephalitis = cNJ; retinitis pigmentosa 3 = cnj;
mental retardation, x-linked 30 = cnJ; oroticaciduria 1 = cnk; fanconi anemia, complementation group n = cnK; japanese
encephalitis = cNK; encephalyocardiomyopathy, mitochondrial, neonatal, due to atp synthase deficiency = cNk; conjunctivitiss =
cNL; vomit recurrent = cnl; pancreatic cancer, susceptibility to, 3 = cnL; deafness, autosomal recessive 6 = cNl; pancreatic
cancer, susceptibility to, 1 = cnM; deafness, autosomal recessive 22 = cnm; hepatic encephalopathy = cNM; epsilon-trimethyllysine
hydroxylase deficiency = cNm; spondyloepimetaphyseal dysplasia, pakistani type = cnN; tenosynovitiss = cNN; deafness,
autosomal recessive 9 = cnn; cardiomyopathy, dilated, 1t = cNn; enterokinase deficiency = cNo; spondylodysplasia and
premature pubarche = cnO; deafness, autosomal recessive 18b = cno; farmer's lung = cNO; deafness, autosomal recessive
84b = cnp; brachyolmia = cnP; pigmented villonodular synovitiss = cNP; deafness, childhood-onset neurosensory, autosomal
recessive 8 = cNp; shortened trunk = cnQ; progressive multifocal leukoencephalopathy = cNQ; deafness, autosomal recessive
10 = cNq; disseminated intravascular coagulatio = cnq; deafness, autosomal dominant 56 = cNr; catch = cNR; dysgnathia
complex = cnr; anovulatio = cnR; microphthalmia, syndromeic 5 = cns; limb-girdle muscular dystrophy type 2h = cnS; osteopetrosis,
autosomal recessive 7 = cNS; achilles tendon injury = cNs; periostitis = cNt; microphthalmia, syndromeic 3 = cnt; osteopetrosis,
mild autosomal recessive form = cNT; mulibrey nanism = cnT; allergic encephalyitis = cnU; polyostotic osteolytic dysplasia,
hereditary expansile = cNU; pituitary hormone deficiency, combined, 6 = cnu; carcinoma of supraglottis = cNu; diabetic
polyneuropathy = cnV; familial paget's disease of bone = cNV; schizophrenia 15 = cnv; psoriatic arthritis, susceptibility
to = cNv; anaplasmosis = cNw; otocephaly = cnw; hyperostosis corticalis deformans juvenilis = cNW; large cell medulloblastoma =
cnW; succinyl-coa:3-oxoacid coa transferase deficiency = cnx; human immunodefiency virus encephalitis = cNx; pyrophosphate
arthritis = cNX; otofaciocervical syndrome 2 = cnX; bleeding disorder due to p2ry12 defect = cny; chromosome 7, trisomy
7p = cnY; immunoglobulin a deficiency 2 = cNY; myelomonocytic leukemia = cNy; immunodeficiency, common variable, 2 =
cNZ; deafness, autosomal dominant 41 = cnz; coloboma of optic nerve = cnZ; borderline lepromatous leprosy = cNz; bronchus
carcinoma = co; hypertriglyceridemia = cO; adenosine deaminase deficiency = CO; severe combined immunodeficiency, autosomal
recessive, t cell-negative, b cell-negative, nk cell-negative, due to adenosine d . . . = Co; hypertrophy of tonsils =
cOa; usher syndrome, type ig = coA; craniofacial deafness hand syndrome = coa; hypermobility syndrome = cOA; klein-waardenberg's
syndrome = cob; retinitis pigmentosa 31 = cOB; transient hypogammaglobulinemia of infancy = cOb; deafness, autosomal
recessive 23 = coB; usher syndrome, type if = coC; immunodeficiency, common variable, 4 = cOc; dystonia, focal, task-specific =
cOC; maturity-onset diabetes of the young, type 9 = coc; multiple sclerosis, susceptibility to, 5 = cOd; psychogenic
dystonia = cOD; epilepsy, female-restricted, with mental retardation = coD; sulfocysteinuria = cod; infantile cerebral
palsy, unspecified = cOE; polysubstance dependencye = coE; adenitis = cOe; leukemia, acute lymphoblastic, susceptibility
to, 3 = coe; skin diseases, viral = cOf; urosepsis = cof; torticollis, familial = cOF; phosphoenolpyruvate carboxykinase
2 deficiency = coF; adrenocortical carcinoma, hereditary = cOG; malignant struma ovari = coG; sprue = cOg; liver abscess,
pyogenic = cog; basal cell carcinoma, susceptibility to, 7 = cOH; pilar and trichilemmal cysts = coH; ornithosis = coh;
breast squamous cell carcinoma = cOh; iga nephropathy, susceptibility to, 1 = cOi; megaesophagus = coI; langerhans cell
sarcoma = coi; trigeminal nerve injuries = cOI; immunoglobulin g4 related sclerosisng disease = cOj; roseolovirus infections =
cOJ; microcephalyic osteodysplastic primordial dwarfism, type ii = coJ; aniridia cerebellar ataxia mental deficiency =
coj; seckel syndrome 2 = coK; keratitis, hereditary = cok; porokeratosis of mibelli = cOK; polyclonal hypergammaglobulinemia =
cOk; actinic cheilitis = cOL; ectopia pupillae = col; inflammatory bowel disease 16 = cOl; osteodysplastic primordial
dwarfism = coL; proprotein convertase 1 3 deficiency = coM; immunodeficiency 16 = cOm; o'donnell pappas syndrome = com;
chronic hyperplastic candidiasis = cOM; sweat gland adenocarcinoma = cON; cardiomyopathy, dilated, 1z = cOn; foveal hypoplasia
1 = con; body mass index quantitative trait locus 12 = coN; sacral defect and anterior sacral meningocele = coO; cardiomyopathy,
familial hypertrophyic, 13 = cOo; cancer, embryonal = cOO; aniridia, type 2 = coo; cardiomyopathy, dilated, 2a = cOp;
knee pain = coP; noninvasive carcinoma ex pleomorphic adenoma = cOP; optic nerve aplasia, bilateral = cop; malignant
stromal tumor = cOQ; cardiomyopathy, dilated, 1ff = cOq; spondylometaphyseal dysplasia with cone-rod dystrophy = coQ;
foveal hypoplasia, isolated = coq; foot diseases = cOR; systemic lupus erythematosus, susceptibility to, 2 = coR; cardiomyopathy,
familial hypertrophyic, 7 = cOr; manifest-latent nystagmus = cor; solitary keratoacanthoma = cOS; fetal microcephaly =
cos; cerebral cavernous malformations 3 = coS; cardiac hypertrophy = cOs; nemaline myopathy 5 = cOt; primary pigmented
nodular adrenocortical disease = coT; congenital ptosis = cot; desmoplastic cerebral astrocytoma of infancy = cOT; cardiomyopathy,
dilated, 1d = cOu; placenta cancer = cou; acrodysostosis 2 with or without hormone resistance = coU; mucinous cystic
tumor of borderline malignancy = cOU; ischaemic cerebral infarction = coV; cyst benign = cOV; tooth agenisi, selective,
3 = cov; cardiomyopathy, familial hypertrophyic, 2 = cOv; choroid plexus carcinoma, childhood = cOW; nonpuerperal galactorrhea =
cow; retinitis pigmentosa 43 = coW; cardiomyopathy, familial restrictive, 3 = cOw; muscular dystrophy, limb-girdle,
type 1f = cOx; adult diffuse astrocytoma = cOX; night blindness, congenital stationary, autosomal dominant 2 = cox; galactorrhea
associated with childbirth = cox; hyperphenylalaninemia with primapterinuria = coy; chondrosarcoma of bone = cOY; ehlers-danlos
syndrome caused by tenascin-x deficiency = cOy; retinitis pigmentosa 40 = coY; recurrent dislocation of joint = cOz;
lymph node cancer = cOZ; cone dystrophy 4 = coZ; organic acidemias = coz; metastatic melanoma = cp; severe combined immunodeficiency
due to adenosine deaminase deficiency = Cp; peyronie's disease = CP; steatohepatitis = cP; ectrodactyly, ectodermal dysplasia,
and cleft lip/palate syndrome 3 = cPA; coenzyme q10 deficiency, primary, 2 = cpA; erythroplakia of mouth = cPa; achromatopsia
5 = cpa; split-hand/foot malformation 4 = cPB; adnexal lesion = cPb; joubert syndrome 22 = cpb; coenzyme q10 deficiency,
primary, 3 = cpB; retinitis pigmentosa 57 = cpc; flat warts = cPc; limb-mammary syndrome = cPC; pancreatic agenisi, congenital =
cpC; chronic cholangitis = cpD; retinal cone dystrophy 3a = cpd; orofacial cleft 8 = cPD; ischemiaa = cPd; sweat gland
neoplasm = cPE; parotid gland cancer = cPe; achromatopsi = cpe; hereditary x-linked recessive spastic paraplegia = cpE;
pneumoniaa = cPf; spinocerebellar ataxia 23 = cpF; skin/hair/eye pigmentation, variation in, 10 = cPF; striatal degeneration,
autosomal dominant = cpf; transient cerebral ischemiaa = cPg; pigmented nodular adrenocortical disease, primary, 3 =
cpg; late cortical cerebellar atrophy = cpG; undifferentiated somatoform disorder = cPG; attention deficit-hyperactivity
disorder, susceptibility to, 7 = cPH; ovary sarcoma = cPh; hypothyroidism in pregnancy = cph; horizontal nystagmus =
cpH; harding ataxia = cpI; triosephosphate isomerase deficiency = cPI; glottis carcinoma = cPi; bone diseases, endocrine =
cpi; basal ganglia calcification, idiopathyic, 5 = cpj; prostate carcinoma in situ = cpJ; triose phosphate isomerase
deficiency = cPJ; papillary cystadenocarcinoma = cPj; localized osteosarcoma = cPk; usher syndrome, type 2a = cpK; thiamine
metabolism dysfunction syndrome 5 (episodic encephalyopathy type) = cPK; diabetic ulcer = cpk; oral dyskinesia = cpL;
adult fibrosarcoma = cpl; cardiomyopathy, familial hypertrophyic, 3 = cPL; multifocal osteogenic sarcoma = cPl; advanced
sleep phase syndrome, familial, 1 = cpM; cardiomyopathy, dilated, 1y = cPM; respiratory system abnormalities = cpm; sarcomatoid
renal cell carcinoma = cPm; nemaline myopathy 4 = cPN; regular astigmatism - corneal = cpn; bone giant cell sarcoma =
cPn; acute q fever = cpN; zebra body myopathy = cPO; addicted to cocaine = cpO; eosinophilic granulomatous polyp = cpo;
gliofibroma = cPo; nasal cavity adenocarcinoma = cPp; proneural glioblastoma = cpp; nemaline myopathy 1 = cPP; recurrent
hypersomnia = cpP; myxopapillary ependymoma = cpq; ileocolic intussusception = cPQ; peroxisome biogenesis disorder 2b =
cpQ; endometrial squamous cell carcinoma = cPq; thiopurine s methyltranferase deficiency = cPR; clear cell chondrosarcoma =
cPr; basal ganglia calcification, idiopathyic, 4 = cpr; peroxisome biogenesis disorder 3a (zellweger) = cpR; ovarian
mucinous neoplasm = cPs; peroxisome biogenesis disorder 3b = cpS; pulmonary capillary hemangiomatosis = cps; gastrointestinal
irritation = cPS; vesiculobullous skin disease = cPT; pdgfrb-associated chronic eosinophilic leukemia = cpt; non-invasive
bladder papillary urothelial neoplasm = cPt; peroxisome biogenesis disorder 11b = cpT; malignant triton tumor = cPu;
thyroid nodule cold = cPU; peroxisome biogenesis disorder 11a (zellweger) = cpU; pyruvate metabolism, inborn errors =
cpu; fibrosarcomatous osteosarcoma = cPv; pyruvate dehydrogenase el-beta deficiency = cpv; peroxisome biogenesis disorder
13a (zellweger) = cpV; toxic diffuse goiter = cPV; spinocerebellar ataxia, autosomal recessive 7 = cPW; maturity-onset
diabetes of the young, type 4 = cpw; peroxisome biogenesis disorder 8b = cpW; atypical follicular adenoma = cPw; lip
cancer = cPx; deafness, autosomal recessive 79 = cPX; charcot-marie-tooth disease, x-linked dominant, 6 = cpx; peroxisome
biogenesis disorder 8a (zellweger) = cpX; herpes simplex encephalyitis, susceptibility to, 3 = cPY; bladder carcinoma
in situ = cPy; peroxisome biogenesis disorder 12a (zellweger) = cpY; hyperplasia of tonsils = cpy; peroxisome biogenesis
disorder 5b = cpZ; multiple myelomaa = cPz; candidiasis, familial, 8 = cPZ; pyruvate dehydrogenase phosphatase deficiency =
cpz; myeloblastic leukemia = cq; epilepsy, idiopathyic generalized = Cq; microalbuminuria = cQ; measles = CQ; dermais
tumor = cQA; peroxisome biogenesis disorder 5a (zellweger) = cqa; psoriasis susceptibility 13 = cQa; glycogen storage
disease, type ixa2 = cqA; peroxisome biogenesis disorder 7a (zellweger) = cqb; progressive familial heart block, type
ib = cQB; glycogen storage disease ixb = cqB; muscular dystrophy, limb-girdle, type 2s = cQb; glycogen storage disease
ixc = cqC; mental retardation, autosomal recessive 13 = cQc; peroxisome biogenesis disorder 7b = cqc; trichorhinophalangeal
syndrome, type iii = cQC; trichorhinophalangeal syndrome type i = cQD; peroxisome biogenesis disorder 10a (zellweger) =
cqd; fibrosis of extraocular muscles, congenital, 2 = cqD; ventricular tachycardia, catecholaminergic polymorphic,
5, with or without muscle weakness = cQd; cerebrohepatorenal syndrome, variant types = cqe; prurigo nodularis = cQE;
trehalase deficiency = cQe; hirschsprung disease ganglioneuroblastoma = cqE; gastrointestinal sensation = cQF; peroxisome
biogenesis disorder 2a (zellweger) = cqf; vasculopathy, retinal, with cerebral leukodystrophy = cQf; neuroblastoma, susceptibility
to, 2 = cqF; peroxisome biogenesis disorder 4a (zellweger) = cqg; chilblain lupus 1 = cQg; urinary incontinence, urge =
cQG; hypoventilation syndrome = cqG; coma, post-head injury = cQh; peroxisome biogenesis disorder 4b = cqh; taste salty =
cQH; chronic respiratory failure = cqH; tertiary hypothyroidism = cQi; sleep-related respiratory failure = cqI; headache
disorders, primary = cQI; refsum disease, adult, 2 = cqi; metatropic dwarfism = cQJ; refsum disease, adult, 1 = cqJ;
familial alcoholism = cQj; amyotrophyic lateral sclerosis 18 = cqj; thyrotropin-releasing hormone resistance, generalized =
cQk; bronchiti = cqK; brachyolmia type 3 = cQK; dimauro disease = cqk; thyrotropin-releasing hormone receptor = cQl;
parastremmatic dwarfism = cQL; 22q11 partial monosomy syndrome = cqL; hyperphosphatasisa with mental retardation = cql;
hyperphosphatasisa with mental retardation syndrome 3 = cqm; spinal muscular atrophy, scapulaperoneal = cQM; placental
abruption = cqM; charcot-marie-tooth disease, axonal, type 2r = cQm; hereditary motor and sensory neuropathy, type iic =
cQN; mabry syndrome = cqn; subacute cutaneous lupus = cQn; testicular infection = cqN; hyperphosphatasisa with mental
retardation syndrome 4 = cqo; brachyolmia type 2 = cQO; lupus erythematosus overlap syndrome = cQo; oculomelic amyoplasia =
cqO; gordon syndrome = cqP; annular erythema = cQp; antral ulcer = cqp; digital arthropathy-brachydactyly, familial =
cQP; multiple congenital anomalies-hypotonia-seizures syndrome 2 = cqQ; spinal muscular atrophy, distal, congenital
nonprogressive = cQQ; mulibrey nanis = cQq; meckel syndrome type 3 = cqq; cerebrovascular trauma = cqr; sodium serum
level quantitative trait locus 1 = cQR; paroxysmal nocturnal hemoglobinuria 1 = cqR; deafness, autosomal recessive 28 =
cQr; nongonococcal urethritis = cqs; achondrogenesis type 1a = cQs; zunich neuroectodermal syndrome = cqS; charcot-marie-tooth
disease, type 2c = cQS; liver failure, infantile, transient = cQt; multiple congenital anomalies-hypotonia-seizures syndrome
1 = cqT; microcephaly 11, primary, autosomal recessive = cqt; focal cortical dysplasia of taylor = cQT; hyperphosphatasisa
with mental retardation syndrome 2 = cqU; deafness, aminoglycoside-induced = cQu; giant cell astrocytoma = cQU; dentin,
secondary = cqu; episodic pain syndrome, familial, 1 = cQv; pontocerebellar hypoplasia type 2 = cQV; heart sarcoma =
cqv; marasmus = cqV; allergy to chemicals = cQw; multiple congenital anomalies-hypotonia-seizures syndrome 3 = cqW; pontocerebellar
hypoplasia type 2a = cQW; phosphoglycerate dehydrogenase deficiency = cqw; pontocerebellar hypoplasia type 4 = cQX; paroxysmal
nocturnal hemoglobinuria 2 = cqX; late onset cerebellar ataxia = cQx; neu laxova syndrome = cqx; congenital lipomatous
overgrowth, vascular malformations, and epidermal nevi = cqY; glycogen storage disease, type ixd = cqy; focal segmental
glomerulosclerosis 2 = cQy; combined oxidative phosphorylation deficiency 3 = cQY; phosphorylase kinase, alpha 2 (liver) =
cqz; hyperthyroidism, nonautoimmune = cQZ; csnblc = cQz; megalencephaly cutis marmorata telangiectatica congenita =
cqZ; psoriasis = cr; tuberculous peritonitis = CR; sleep initiation and maintenance disorders = Cr; endothelial dysfunction =
cR; dystonia-parkinsonism, adult-onset = crA; hyperthyroidism, familial gestational = cRa; cowden syndrome 5 = cra;
cardiomyopathy, dilated, 1g = cRA; thyroid carcinoma with thyrotoxicosis = cRb; infected ascites = crb; cardiomyopathy,
familial hypertrophyic, 9 = cRB; nbia2b = crB; thyroid adenoma, hyperfunctioning = cRc; external auditory canal ceruminous
adenoma = crc; karak syndrome = crC; muscular dystrophy, limb-girdle, type 2j = cRC; stage iv endometrial cancer = crd;
myopathy, early-onset, with fatal cardiomyopathy = cRD; swallowing problem = crD; congenital hyperthyroidism = cRd; obstructive
chronic bronchitis with (acute) exacerbation = cre; neonatal hyperthyroidism = cRe; platelet-activating factor acetylhydrolase
deficiency = crE; tibial muscular dystrophy = cRE; familial restrictive cardiomyopathy = cRF; subclinical hyperthyroidism =
cRf; breast mucinous carcinoma = crf; intestinal atresia = crF; orbital congestion = cRg; centriacinar emphysema =
crG; dystransthyretinemiac euthyroidal hyperthyroxinemia = cRG; ceruminoma = crg; amyloid of vitreous = cRH; agammaglobulinemia
7, autosomal recessive = crh; diffuse goiter = cRh; hematoma, epidural, spinal = crH; thrombophilia, familial, due to
decreased release of tissue plasminogen activator = crI; age-related amyloidosis = cRI; toxic goiter = cRi; ataxia-oculomotor
apraxia 3 = cri; embolism and thrombosis = crJ; familial polyneuropathy = cRJ; heaves = crj; plummer's disease = cRj;
postoperative adhesion = crK; gastrointestinal dysfunction = cRK; neonatal thyrotoxicosis = cRk; cystic breast disease =
crk; chronic cerebral ischemia = cRl; lethal congenital contractural syndrome 3 = crl; cardiac tamponade = crL; hereditary
cardiac amyloidosis = cRL; absent peristalsis = cRm; hepatic amyloidosis = cRM; cholesterol embolism = crM; synostosis,
carpal, with dysplastic elbow joints and brachydactyly = crm; astrocytic hamartoma = crn; congenital pelviureteric junction
obstruction = cRn; eye lesion = cRN; strongylida infections = crN; epileptic encephalyopathy, early infantile, 12 = crO;
exudative vitreoretinopathy 5 = cRo; ring dermaoid of cornea = cro; amyloid myopathy = cRO; single atrium = crp; mental
retardation, x-linked 58 = cRp; migrating partial seizures in infancy = crP; monoparesis - arm = cRP; disabling disease =
cRQ; deafness, autosomal recessive 98 = cRq; cataract, posterior polar, 4 = crq; auriculocondylar syndrome 2 = crQ;
sudden infant death with dysgenesis of the testes syndrome = cRr; edema vascular = crr; leukonychia totalis = crR; early
satiety = cRR; autoinflammation, antibody deficiency, and immune dysregulation, plcg2-associated = crS; presbyopia =
cRS; vascular aneurysm = crs; spinocerebellar ataxia 11 = cRs; lissencephaly 3 = cRT; familial cold autoinflammatory
syndrome 3 = crT; vascular fragility = crt; mitochondrial complex ili deficiency, nuclear type 2 = cRt; nephronophthisis
12 = cRu; benign neurilemmoma = cru; urticaria due to cold = crU; macrothrombocytopenia, autosomal dominant, tubbl-related =
cRU; alzheimer disease 19 = crV; dextrocardia = crv; short-rib thoracic dysplasia 4 with or without polydactyly = cRv;
cortical dysplasia, complex, with other brain malformations 5 = cRV; multiple cysts = crw; gou = cRw; shipyard ey = crW;
polymicrogyria, symmetric or asymmetric = cRW; adenosine triphosphate, elevated, of erythrocytes = crx; polymicrogyria,
asymmetric = cRX; retinitis pigmentosa 51 = cRx; epidermaolysis bullosa simplex, ogna type = crX; fibrosis of extraocular
muscles, congenital, 3a, with or without extraocular involvement = cRY; arrhythmogenic right ventricular dysplasia, familial,
9 = cry; epidermaolysis bullosa simplex with pyloric atresia = crY; bardet-biedl syndrome 8 = cRy; mental retardation,
autosomal recessive 39 = cRz; fleck retina, familial benign = crz; muscular dystrophy, limb-girdle, type 2q = crZ; cortical
dysplasia, complex, with other brain malformations 1 = cRZ; scid due to ada deficiency, delayed onset = Cs; gamma aminobutyric
acid transaminase deficiency = cs; carotid atherosclerosis = cS; polyarteritis nodosa = CS; chromosome 17, trisomy 17p =
csA; infection by anisakis larva = csa; congenital absence of vagina = cSA; dystonia musculorum deformans 4 = cSa;
infection by anisakidae = csb; rufous oculocutaneous albinism = cSB; leukodystrophy, hypomyelinating, 6 = cSb; disorder
of hand = csB; skin/hair/eye pigmentation, variation in, 11 = cSC; multiple cranial neuropathy = csC; anisakiasis = csc;
cortical dysplasia, complex, with other brain malformations 4 = cSc; osteopetrosis, autosomal recessive 6 = csd; combined
oxidative phosphorylation deficiency 4 = cSd; colorectal cancer, hereditary nonpolyposis, type 4 = csD; arthrogryposis
multiplex congenita, distal, x-linked = cSD; generalized osteopenia = cse; retinitis pigmentosa 14 = cSe; epileptic encephalyopathy,
early infantile, 10 = csE; mental retardation, x-linked, syndromeic, nascimento type = cSE; dna repair-deficiency disorders =
csF; alpha-2-plasmin inhibitor deficiency = csf; leber congenital amaurosis 15 = cSf; kaufman oculocerebrofacial syndrome =
cSF; bone marrow oedema = csg; pancreatic triacylglycerol lipase deficiency = csG; chromosome 15q, trisomy = cSG; central
topographic island = cSg; encephalyomyeliti = csH; mental retardation, autosomal recessive 7 = cSh; blepharophimosis-ptosis-intellectual
disability syndrome = cSH; axillary veins thrombosis = csh; saethre-chotzen syndrome with eyelid anomalies = cSi; terminal
cancer = csI; streptococcal infection of skin = csi; schnyder corneal dystrophy = cSI; ichthyosis, congenital, autosomal
recessive 10 = csJ; amyotrophyic lateral sclerosis 15, with or without frontotemporal dementia = cSJ; robinow sorauf
syndrome = cSj; membranous conjunctivitis = csj; baller-gerold syndrome = cSk; pneumoniac plague = csk; spastic paraplegia
39, autosomal recessive = csK; johanson-blizzard syndrome = cSK; lipodystrophy, familial partial, type 4 = csl; chorioretinal
dystrophy, spinocerebellar ataxia, and hypogonadotropic hypogonadism = csL; facial ectodermal dysplasia = cSl; parkinson
disease 5, autosomal dominant = cSL; neurodegeneration with optic atrophy, childhood-onset = cSM; cardiomyopathy, dilated,
1p = csm; pyridoxamine 5-prime-phosphate oxidase deficiency = csM; diabetic embryopathy = cSm; deafness, autosomal recessive
70 = csN; cardiomyopathy, familial hypertrophyic, 18 = csn; body mass index quantitative trait locus 4 = cSN; tyrosine
kinase 2 deficiency = cSn; combined oxidative phosphorylation deficiency 13 = csO; bruck syndrome 2 = cso; hyperbilirubinemia,
transient familial neonatal = cSO; encephalyomyopathy = cSo; small intestine cancer = cSP; spastic paraplegia 2, x-linked =
csp; basosquamous carcinoma = cSp; short stature, onychodysplasia, facial dysmorphism, and hypotrichosis = csP; pelizaeus-merzbacher
disease, atypical = csq; opiate withdrawal symptoms = cSQ; poisoning by fluorouracil = cSq; aberrant right subclavian
artery = csQ; medullary cystic kidney disease 2 = cSR; occult macular dystrophy = csr; rectal cancer stage = cSr; single
umbilical artery = csR; glomerulocystic kidney disease with hyperuricemia and isosthenuria = cSS; oculocutaneous albinism
type 1b = cSs; hypergranular promyelocytic leukemia = css; aberrant subclavian artery = csS; premature ovarian failure
2b = csT; hemophagocytic lymphohistiocytosis, familial, 3 = cST; loosening of prosthesis = cst; oculocutaneous albinism
type 3 = cSt; herpes simplex encephalyitis, susceptibility to, 1 = cSU; charcot-marie-tooth disease, type 2e = csu; albinism,
oculocutaneous, type i, temperature-sensitive = cSu; dowling-degos disease 2 = csU; stage ii melanoma = cSv; charcot-marie-tooth
disease, demyelinating, type ie = csv; dowling-degos disease 4 = csV; immunodeficiency with hyper-igm, type 5 = cSV;
tyrosinase-negative albinism = cSw; amyotrophy, hereditary neuralgic = csw; beta-ureidopropionase deficiency = cSW; colorectal
cancer, susceptibility to, 10 = csW; hepatic venoocclusive disease with immunodeficiency = csx; mandibular hypoplasia,
deafness, progeroid features, and lipodystrophy syndrome = csX; autoimmune endocrine disease = cSx; adenosquamous pancreas
carcinoma = cSX; colorectal cancer, susceptibility to, 12 = csY; mental retardation, x-linked, syndromeic 14 = cSY; charcot-marie-tooth
disease and deafness = csy; secondary malignant neoplasm of spleen = cSy; mitochondrial complex iii deficiency, nuclear
type 5 = cSZ; pes cavus = csz; facial dysmorphism, immunodeficiency, livedo, and short stature = csZ; iris disease =
cSz; xeroderma pigmentosum = CT; depressive disorder = ct; allergic disposition = cT; priapism, familial idiopathyic =
Ct; mitochondrial complex iii deficiency, nuclear type 4 = cTa; progressive external ophthalmoplegia with mitochondrial
dna deletions, autosomal recessive = cta; total retinal detachment = cTA; muscular dystrophy-dy stroglycanopathy (limb-girdle),
type c, 3 = ctA; muscular dystrophy-dystroglycanopathy (congenital with mental retardation), type b, 3 = ctB; progressive
external ophthalmoplegia with mitochondrial dna deletions, autosomal dominant, 1 = ctb; urocanase deficiency = cTb; clinically
significant macular edema = cTB; muscular dystrophy-dystroglycanopathy (congenital with brain and eye anomalies), type
a, 3 = ctC; acute bacterial endocarditis = cTC; intermittent ataxia = cTc; mitochondrial dna depletion syndrome 4b (mngie
type) = ctc; familial porphyria cutanea tarda = cTd; muscular dystrophy-dystroglycanopathy (congenital with brain and
eye anomalies), type a, 8 = ctD; abnormal yolk sac = cTD; mitochondrial dna depletion syndrome 4a (alpers type) = ctd;
homozygous porphyria cutanea tarda = cTe; rotator cuff syndrome = cTE; hydrocephalyu = ctE; ataxia neuropathy spectrum =
cte; epilepsyia partialis continua = ctf; disease attributes = cTf; tuberculosis = cTF; muscular dystrophy-dystroglycanopathy
(congenital with brain and eye anomalies), type a, 12 = ctF; spinocerebellar ataxia with epilepsy = ctg; keratosis linearis
with ichthyosis congenita and sclerosisng keratoderma = ctG; rubeosis iridis = cTG; hyperactive child = cTg; central
nervous system tuberculosis = cTH; muscular dystrophy-dystroglycanopathy (limb-girdle), type c, 1 = ctH; hepatoerythropoietic
porphyria = cTh; infertility/subfertility = cth; muscular dystrophy-dystroglycanopathy (congenital with mental retardation),
type b, 1 = ctI; progressive external ophthalmoplegia with mitochondrial dna deletions, autosomal dominant, 4 = cti;
cutaneous porphyri = cTi; neovascular glaucoma = cTI; deafness, autosomal recessive 18 = cTj; muscular dystrophy-dystroglycanopathy
(limb-girdle), type c, 2 = ctJ; xeroderma pigmentosum, variant type = ctj; central retinal vein occlusion = cTJ; xanthogranulomatous
cholecystitis = cTK; mandibulofacial dysostosis, treacher collins type, autosomal recessive = ctk; usher syndrome, type
ic = cTk; muscular dystrophy-dystroglycanopathy (congenital with mental retardation), type b, 2 = ctK; muscular dystrophy-dystroglycanopathy
(congenital with brain and eye anomalies), type a, 2 = ctL; treacher collins syndrome 2 = ctl; retinitis pigmentosa 39 =
cTl; left ventricular diastolic dysfunction = cTL; erythrocytosis, familial, 2 = cTM; anophthalmia with pulmonary hypoplasia =
ctm; agricultural workers' diseases = ctM; mental retardation, x-linked 99 = cTm; leukodystrophy, hypomyelinating,
with hypodontia and hypogonadotropic hypogonadism = ctn; inferior myocardial infarction = cTN; uv-sensitive syndrome
3 = cTn; microvascular complications of diabetes, susceptibility to, 5 = ctN; enzyme activity finding = ctO; arthrogryposis,
renal dysfunction, and cholestasis 2 = cTO; leukodystrophy, hypomyelinating, 8, with or without oligodontia and/or hypogonadotropic
hypogonadism = cto; amyotrophyic lateral sclerosis 8 = cTo; pesticide poisoning = ctP; hypocortisolism secondary to another
disorder = ctp; vitamin k-dependent clotting factors, combined deficiency of, type 2 = cTP; spinal muscular atrophy,
proximal, adult, autosomal dominant = cTp; antley-bixler syndrome with genital anomalies and disordered steroidogenesis =
ctQ; myopathy, x-linked, with excessive autophagy = cTQ; spinal muscular atrophy, late-onset, finkel type = cTq; melanoderma =
ctq; microphthalmia, syndromeic 11 = cTr; parkinson disease 17 = cTR; periodontitis, aggressive, 2 = ctR; excessive
drinking = ctr; cardiomyopathy, familial hypertrophyic, 15 = cTs; pituitary hormone deficiency, combined, 1 = ctS; obesity
hypoventilation syndrome = cts; spastic paraplegia 53, autosomal recessive = cTS; progressive hearing loss stapes fixation =
ctT; syndactyly of toes = cTT; cardiomyopathy, dilated, 1w = cTt; conn adenoma = ctt; amyotrophyic lateral sclerosis
14, with or without frontotemporal dementia = cTu; gamma-cystathionase deficiency = cTU; cervical abnormality = ctU;
micronodular adrenal hyperplasia = ctu; idiopathyic hypopituitarism = ctv; deafness, autosomal dominant 15 = ctV; atherosclerosis,
susceptibility to = cTv; keratoconus 1 = cTV; craniofacial anomalies and anterior segment dysgenesis syndrome = cTW;
apparent life-threatening event = ctw; polyembryoma = ctW; periodontal atrophy = cTw; adrenal cortex disease = ctx; mediastinum
seminoma = ctX; sarcoid type granuloma = cTx; microphthalmia, isolated 2 = cTX; microphthalmia, isolated, with coloboma
3 = cTY; critical lower limb ischemia = cTy; pancreatic endocrine carcinoma = cty; phenylketonuri = ctY; microphthalmia,
cataracts, and iris abnormalities = cTZ; subvalvular aortic stenosis = ctz; wilms tumor and radial bilateral aplasia =
ctZ; retrograde menstruation = cTz; reticular dysgenesis = CU; respiratory syncytial virus (rsv) = cU; x-linked chronic
granulomatous disease = Cu; cocaine-related disorders = cu; von willebrand disease, type iic = cU; brazilian purpuric
fever = cuA; cataract, nuclear total = cUA; sympathyomimetic disorder = cua; wiskott-aldrich syndrome 2 = cUB; dilated
cardiomyopathy secondary to metabolic disorder = cub; platelet aggregation, spontaneous = cUb; noise-induced temporary
threshold shift = cuB; central nervous system vasculitis = cuC; hypertensive encephalyopathy = cuc; von willebrand disease,
type iid = cUc; pseudohypoaldosteronism, type iic = cUC; epicondylitis = cuD; pseudohypoaldosteronism, type iib = cUD;
congenital von willebrand's disease = cUd; carotid intimal medial thickness 1 = cud; pseudohypoaldosteronism, type iia =
cUE; montreal platelet syndrome = cUe; hereditary protein c deficiency = cuE; childhood myelodysplastic syndrome =
cue; osteogenesis imperfecta, type xv = cUF; homozygous protein c deficiency = cuF; osteogenesis imperfecta, type ix =
cuf; acquired von willebrand's disease = cUf; partial stenosis = cUg; brain stem cancer = cug; heterozygous protein
c deficiency = cuG; bone mineral density quantitative trait locus 16 = cUG; homozygous protein s deficiency = cuH; thrombocytopenia,
x-linked, intermittent = cUh; odontoonychodermal dysplasia = cUH; maple syrup urine disease, mild variant = cuh; tooth
agenisi, selective, 4 = cUI; bardet-biedl syndrome 15 = cUi; homozygous variegate porphyria = cui; umbilical bleeding =
cuI; hypogonadotropic hypogonadism 14 with or without anosmia = cUj; variegate porphyri = cuj; preeclamptic toxemia =
cul; schopf-schulz-passarge syndrome = cUJ; odonto-onycho-dermal dysplasia = cUK; aleutian mink disease = cuK; bartonellosis =
cuk; nephronophthisis 13 = cUk; cranioectodermal dysplasia 4 = cUl; split-hand/foot malformation 6 = cUL; epilepsy,
progressive myoclonic 5 = cuL; spinocerebellar ataxia 12 = cul; diverticulitis = cUM; short-rib thoracic dysplasia 5
with or without polydactyly = cUm; tremor, limb = cum; myopia 22, autosomal dominant = cuM; progressive familial heart
block, type ii = cun; short-rib thoracic dysplasia 11 with or without polydactyly = cUn; tetra-amelia autosomal recessive =
cUN; cardiomyopathy, familial hypertrophyic, 6 = cuN; cranioectodermal dysplasia 2 = cUo; glycogen storage disease
of heart, lethal congenital = cuO; multiple endocrine neoplasmia type = cuo; sex reversal, female, with dysgenesis of
kidneys, adrenals, and lungs = cUO; renpenning syndrome 1 = cup; fuhrmann syndrome = cUP; short-rib thoracic dysplasia
7 with or without polydactyly = cUp; fasciculoventricular accessory pathyway = cuP; glaucoma 1, open angle, g = cUq;
von willebrand disease type 2a = cuq; diabetes in old age = cuQ; al awadi syndrome = cUQ; dyskeratosis congenita, autosomal
recessive, 3 = cUR; neurodegeneration with brain iron accumulation 5 = cUr; carbohydrate metabolic disorder = cuR; drug-induced
immune thrombocytopenia = cur; short-rib thoracic dysplasia 8 with or without polydactyly = cUs; capsule opacification =
cUS; atrial myxoma, familial = cuS; prolactinom = cus; retinitis pigmentosa 36 = cut; microcephaly 2, primary, autosomal
recessive, with or without cortical malformations = cUt; acrodysostosis 1 with or without hormone resistance = cuT; nephrotic
syndrome, type 4 = cUT; meacham syndrome = cUU; brittle cornea syndrome 2 = cuu; kuzniecky syndrome = cUu; large cell
calcifying sertoli cell tumor = cuU; nuclear sclerosis = cuv; amelogenesis imperfecta, hypomaturation type, iia3 = cUv;
meacham winn culler syndrome = cUV; immunodeficiency, common variable, 9 = cuV; cerebellar ataxia, mental retardation,
and dysequilibrium syndrome 2 = cUw; chromosome 2, trisomy 2p = cuw; bilateral wilms tumor = cUW; spinocerebellar ataxia
14 = cuW; infantile convulsions and paroxysmal choreoathetosis, familial = cuX; euthymic mood = cux; defect of diaphragm =
cUX; deafness, autosomal dominant 6 = cUx; retrograde amnesia = cuy; childhood kidney neoplasm = cUY; familial paroxysmal
kinesigenic dyskinesia = cuY; cataract 41 = cUy; wolfram-like syndrome, autosomal dominant = cUz; severe combined immunodeficiency,
autosomal recessive, t cell-negative, b cell-negative, nk cell-positive = cuZ; mycoplasma pneumoniae infections = cuz;
skull base meningioma = cUZ; dyskinesia, drug-induced = cv; lymphocytosis = Cv; alzheimer disease 18 = CV; channelopathyies =
cV; massive hepatic necrosis = cva; diaphragmatic eventration = cVA; malignant epithelial mesothelioma = cVa; hypogonadotropic
hypogonadism 3 with or without anosmia = cvA; aortic aneurysm, familial thoracic 8 = cvb; kallmann syndrome, type 3,
recessive = cvB; spinocerebellar ataxia, autosomal recessive 12 = cVb; spastic paraplegia 33, autosomal dominant = cVB;
macular dystrophy, retinal, 2 = cvC; dystonia 16 = cvc; dextrocardi = cVC; scarred macula = cVc; ciliary dyskinesia,
primary, 22 = cVD; microtia-anotia = cvd; retinitis pigmentosa 41 = cvD; abiotrophy = cVd; stargardt disease 4 = cvE;
angioedema induced by ace inhibitors, susceptibility to = cVe; fetal resorption = cve; polydactyly, postaxial, type aG =
cVE; macroprolactinoma = cvf; cone-rod dystrophy 12 = cvF; angioedem = cVf; tooth agenisi, selective, 5 = cVF; microcephaly
10, primary, autosomal recessive = cVG; refractory angina = cvG; idiopathyic hyperprolactinemia = cvg; nephronophthisis-like
nephropathy 1 = cVg; nephrolithiasis, uric acid, susceptibility to = cVH; invasive pituitary adenoma = cvh; pituitary
mass = cvH; upper gi cancer = cVh; coloboma, uveal, with cleft lip and palate and mental retardation = cVi; thrombophilia
due to protein s deficiency, autosomal dominant = cvI; multiple fibroadenomas of the breast = cvi; nephronophthisis 14 =
cVI; huntington disease-like 1 = cvj; thrombophilia due to protein s deficiency, autosomal recessive = cvJ; myopathy,
lactic acidosis, and sideroblastic anemia 2 = cVj; retinitis pigmentosa 58 = cVJ; spinocerebellar ataxia, autosomal recessive
5 = cVK; protein z deficiency = cvK; t cell immunodeficiency primary = cVk; spongiform encephalyopathy with neuropsychiatric
features = cvk; plasma cell myeloma/plasmacytoma = cVl; retinitis pigmentosa 18 = cvL; myopia 21, autosomal dominant =
cVL; amyloidosis, cerebral, with spongiform encephalyopathy = cvl; cns degeneration = cvm; mental retardation, x-linked,
znf711-related = cVM; skeletal defects, genital hypoplasia, and mental retardation = cVm; retinitis pigmentosa fl = cvM;
retinitis pigmentosa 60 = cvN; partial tetrasomy = cvn; chromosome 11q43-944 deletion syndrome = cVn; seborrhea-like
dermatitis with psoriasisform elements = cVN; mental retardation, autosomal dominant 22 = cVo; mental retardation, x-linked
45 = cVO; retinitis pigmentosa 13 = cvO; chromosome 15q, tetrasomy = cvo; peripheral retinal degeneration = cvP; acromelic
frontonasal dysostosis = cVP; human transmissible spongiform encephalyopathyies, inherited = cvp; primrose syndrome =
cVp; immunodeficiency-centromeric instability-facial anomalies syndrome 2 = cVq; retinitis pigmentosa 7 = cvQ; chronic
wasting disease = cvq; patterned dystrophy of retinal pigment epithelium = cvR; scrapie = cvr; wieacker syndrome = cVr;
cystoid macular edem = cvS; morphine dependency = cVs; kuru = cvs; mental retardation, x-linked 91 = cVt; arts syndrome =
cvT; thrombophilia, hereditary, due to protein c deficiency, autosomal recessive = cvt; thrombophilia, hereditary,
due to protein c deficiency, autosomal dominant = cvu; optic atrophy polyneuropathy deafness = cvU; mental retardation,
x-linked, syndromeic, raymond type = cVu; congenital thrombotic disease, due to protein c deficiency = cvv; deafness,
x-linked 1 = cvV; corneal dystrophy, fuchs endothelial, 6 = cVv; renal thrombosis = cvw; phosphoribosylpyrophosphate
synthetase superactivity = cvW; corneal dystrophy, posterior polymorphous, 3 = cVw; congenital ptosi = cVx; hypoglycemiac
coma = cvx; dystonia 18 = cvX; hypogonadotropic hypogonadism 4 with or without anosmia = cvy; nager acrofacial dysostosi =
cVy; convulsions, benign familial infantile, 2 = cvY; kallmann syndrome 4 = cvz; acrofacial dysostosis, nager type =
cvZ; diaphragmatic hernia 3 = cVz; recurrent infections (sinusitis and bacterial pneumonia and meningitis) = Cw; reflex,
abnormal = cw; familial alzheimer disease = CW; coronary atherosclerosis = cW; congenital absence of jaw = cwa; dentigerous
cyst = cwA; jaw cysts = cwB; autosomal dominant hereditary pancreatitis = cwb; pancreatic symptom = cwc; 9q22.3 microdeletion =
cwC; mental retardation, autosomal recessive 1 = cwd; open spina bifida = cwD; lenz majewski hyperostotic dwarfism =
cwE; gastroenteriti = cwe; charcot-marie-tooth disease, demyelinating, type 4f = cwf; macrocephaly autism syndrome =
cwF; metachromatic leukodystrophy due to saposin b deficiency = cwg; vacterl hydrocephaly = cwG; gaucher disease, atypical,
due to saposin c deficiency = cwh; vater association with macrocephaly and ventriculomegaly = cwH; krabbe disease, atypical,
due to saposin a deficiency = cwi; pten hamartoma tumor syndrome with granular cell tumor = cwI; proteus-like syndrome
(disorder) = cwJ; phosphoserine aminotransferase deficiency = cwj; cronkite-canada syndrome = cwK; linitis plastica =
cwk; cardiomyopathy, dilated, 1u = cwl; arteriovenous graft = cwL; endometritis = cwM; alzheimer disease, familial, type
3 = cwm; ampulla of vater adenocarcinoma = cwN; alzheimer disease, familial, 3, with spastic paraparesis and apraxia =
cwn; alzheimer disease, familial, 3, with spastic paraparesis and unusual plaques = cwo; multilocular clear cell renal
cell carcinoma = cwO; acne inversa, familial, 3 = cwp; cervical endometrioid adenocarcinoma = cwP; coliti = cwQ; frontal
lobe syndrome = cwq; diabetes mellitus, permanent neonatal, with cerebellar agenisi = cwR; disorientation = cwr; kluver-bucy
syndrome = cws; asthma-related traits, susceptibility to, 1 = cwS; cardiomyopathy, dilated, lv = cwt; medulloblastoma
of cerebellum = cwT; acute inflammatory demyelinating polyneuropathy = cwU; acne inversa, familial, 2 = cwu; ovarian
dysgenesis 3 = cwv; asthma, nasal polyps, and aspirin intolerance = cwV; asthma, aspirin-induced, susceptibility to =
cwW; deficiency of phosphoserine phosphatase = cww; carcinoma-associated retinopathy = cwx; body temperature changes =
cwX; holoprosencephaly 7 = cwy; capillariasis = cwY; eccrine carcinoma = cwZ; basal cell carcinoma, susceptibility
to, 1 = cwz; malignant neoplasm of female breast = cX; cardiac dilatation = CX; partial adenosine deaminase deficiency =
Cx; psychomotor disorders = cx; jansen type metaphyseal chondrodysplasia = cxa; hepatitis c virus, susceptibility to =
cxA; hypophosphatasisa, adult = cxb; metastatic tumors of the ewing's family = cxB; primary hypoparathyroidism = cxc;
nephrotic syndrome, type 6 = cxC; primary hyperthyroidism = cxd; deafness, autosomal recessive 84a = cxD; lipodystrophy,
congenital generalized, type 4 = cxE; senile osteoporosis = cxe; adynamic bone disease = cxf; 6-pyruvoyl-tetrahydropterin
synthase deficiency = cxF; verheij syndrome = cxG; osteitis fibrosa = cxg; chondrodysplasia blomstrand type = cxh; myopathy
with lactic acidosis and sideroblastic anemia = cxH; failure of tooth eruption, primary = cxi; allergy to fish = cxI;
poliomayelitis, paralytic = cxJ; eiken skeletal dysplasia = cxj; failure of tooth eruption = cxk; rosselli-gulienetti
syndrome = cxK; chondrodysplasia blomstrand typ = cxl; orofacial cleft 7 = cxL; brachydactyly, type e2 = cxm; ectodermal
dysplasia-syndactyly syndrome 1 = cxM; sclerocornea, autosomal recessive = cxN; bone carcinoma = cxn; angiomyoma = cxo;
cutis laxa, autosomal recessive, type iib = cxO; cutis laxa, autosomal recessive, type iiib = cxP; sclerosisng hepatic
carcinoma = cxp; male turner syndrome = cxq; posturing = cxQ; stomach atrophy = cxr; hypertensive heart and renal disease =
cxR; noonan like syndrome = cxs; hyperandrogenis = cxS; martsolf syndrome = cxT; heart displacement = cxt; cleft leaflet
of mitral valve = cxu; warburg micro syndrome 3 = cxU; warburg micro syndrome = cxV; hereditary oculoleptomeningeal amyloid
angiopathy = cxv; secondary malignant neoplasm of lymph nodes of neck = cxW; communicating hydrocephalyus = cxw; cone-rod
dystrophy 18 = cxx; choanal atresia and lymphedema = cxx; hereditary hemorrhagic telangiectasis = cxy; smith-mccort dysplasia
2 = cxY; infection - suppurative = cxz; mental retardation, x-linked 72 = cxZ; pricking of skin = Cy; lymphangiosis carcinomatosa =
CY; tumor vasculature = cY; ischemia = cy; warburg micro syndrome 2 = cya; cone-rod dystrophy 11 = cyA; martin-probst
deafness-mental retardation syndrome = cyb; macular degeneration, age-related, 6 = cyB; microcephaly with mental retardation
and digital anomalies = cyC; kaposi sarcoma, susceptibility to = cyc; tarp syndrome = cyD; neutrophyil immunodeficiency
syndrome = cyd; grade iii meningioma = cye; cardiomyopathy, dilated, 1dd = cyE; cornealia de lange syndrome 4 = cyf;
alopecia, neurologic defects, and endocrinopathy syndrome = cyF; nijmegen breakage syndrome-like disorder = cyg; carcinoma
of sigmoid colon = cyG; spermatogenic failure, nonobstructive, y-linked = cyH; mirror movements 2 = cyh; fanconi anemia,
complementation group o = cyi; retinitis pigmentosa 66 = cyI; breast-ovarian cancer, familial, susceptibility to, 3 =
cyj; retinal dystrophy, iris coloboma, and comedogenic acne syndrome = cyJ; keratomalacia = cyK; breast-ovarian cancer,
familial, susceptibility to, 4 = cyk; adams-oliver syndrome 3 = cyL; noonan syndrome 5 = cyl; visceral myopathy = cyM;
leopard syndrome, 2 = cym; melanoma-associated retinopathy = cyN; benign struma ovarii = cyn; leber congenital amaurosis
13 = cyO; alpha/beta t-cell lymphopenia with gamma/delta t-cell expansion, severe cytomaegalovirus infection, and autoimmunity =
cyo; combined cellular and humoral immune defects with granulomas = cyp; leber congenital amaurosis 3 = cyP; encephalyopathy,
acute, infection-induced, susceptibility to, 3 = cyq; deafness, autosomal recessive, 24 = cyQ; tick-borne diseases =
cyr; spastic paraplegia 31, autosomal dominant = cyR; neuronopathy, distal hereditary motor, type vb = cyS; hyperchloremia =
cys; myasthenic syndrome, congenital, with facial dysmorphism, associated with acetylcholine receptor deficiency =
cyt; spastic paraplegia 72, autosomal recessive = cyT; pancreas exocrine = cyU; microphthalmia, syndromeic 12 = cyu;
norman roberts lissencephaly syndrome = cyV; pontocerebellar hypoplasia type = cyv; bilateral superior oblique palsy =
cyW; capillary malformation-arteriovenous malformation = cyw; capillary malformation without arteriovenous malformation =
cyx; hyperuricemiac nephropathy, familial juvenile 2 = cyX; histiocytoid hemangioma = cyy; brachydactyly with hypertension =
cyY; high-renin essential hypertension = cyZ; microphthalmia, isolated 3 = cyz; macrocytosis = Cz; hypertension = cz;
candidiasis = CZ; ganglion cysts = cZ; cutaneous lichen amyloidosis = cza; macrocephaly, alopecia, cutis laxa, and scoliosis =
czA; mucosal neuromas = czb; popliteal pterygium syndrome, lethal type = czB; papillary carcinoma of the breast = czc;
cocoon syndrome = czC; thyroid nodule solitary = czd; noonan syndrome 8 = czD; bothnia retinal dystrophy = czE; thyroid
hyalinizing trabecular adenoma = cze; congenital disorder of glycosylation, type in = czf; newfoundland rod-cone dystrophy =
czF; combined oxidative phosphorylation deficiency 11 = czG; bare lymphocyte syndrome, type ii, complementation group
c = czg; bare lymphocyte syndrome, type ii, complementation group e = czh; bacterial sinusitis = czH; mitchell-riley
syndrome = czi; aicardi-goutieres syndrome 4 = czI; aicardi-goutieres syndrome 2 = czJ; bare lymphocyte syndrome, type
ii, complementation group b = czj; aicardi-goutieres syndrome 3 = czK; bare lymphocyte syndrome, type ii, complementation
group d = czk; leukoencephalyopathy, cystic, without megalencephaly = czL; retinitis pigmentosa 44 = czl; postmortem
changes = czm; autoimmune thrombocytopeniac purpur = czM; macrocephaly, macrosomia, facial dysmorphism syndrome = czN;
bradyopsia = czn; rh-null, regulator type = czo; hereditary renal cancer associated 1 = czO; stomatocytosis i = czp;
plagu = czP; keratosis palmoplantaris with esophageal cancer = czq; ataxia, sensory, autosomal dominant = czQ; rh-null
disease, amorph type = czr; moyamoya disease 2 = czR; vesicoureteral reflux 2 = czS; hemolytic disease of fetus or newborn
due to rhd isoimmunization = czs; night blindness, congenital stationary, autosomal dominant 1 = czt; gaze palsy, familial
horizontal, with progressive scoliosis = czT; retinitis pigmentosa 4 = czu; horizontal gaze palsy = czU; scotopic sensitivity =
czv; kohlschutter tonz syndrome = czV; retina--diseases = czw; retinitis pigmentosa 7, digenic = czW; sarcoma, avian =
czx; robinow syndrome, autosomal recessive = czX; acrodermatitis atrophyicans chronica = czy; retinitis pigmentosa
2 = czY; ebola hemorrhagic feve = czz; chromosome xp11.3 deletion syndrome = czZ; glioblastoma multiforme = d; wounds
and injuries = D; severe combined immunodeficiency = dA; carcinoma, lewis lung = DA; diseases in twins = da; relapsing-remitting
multiple sclerosis = Da; hypoglossal nerve injuries = DB; brain cancer = dB; papillary carcinoma = Db; drusen = db; reticulate
acropigmentation of kitamur = Dc; microspherophakia = DC; coinfection = dc; aortic atherosclerosis = dC; aqueous humor
disorders = DD; cancer recurrence = Dd; atherosclerosis = dD; synovial cyst = dd; chronic ulcer = De; weill-marchesani
syndrome = DE; plague = dE; photoreceptor degeneration = de; neutrophyilia = df; edwards syndrome = Df; adrenoleukodystrophy =
dF; airway disease = DF; external hyperostosis = Dg; hyperglycemia = dG; familial disease = dg; ataxia with vitamin
e deficiency = DG; mammary carcinoma, human = Dh; primary open angle glaucoma = dH; complement factor h deficiency =
DH; vascular inflammations = dh; gastric ulcer = dI; von willebrand disease type 2b = DI; inflammatory joint disease =
Di; memory loss = di; osteophyte = Dj; thrombotic thrombocytopeniac purpura, acquired = DJ; high density lipoprotein
deficiency = dj; amnestic disorder = du; middle east respiratory syndrome = dk; membranous glomerulonephritis = dK; acute
disease = DK; inflammatory skin and bowel disease, neonatal = Dk; congenital deficiency = DL; adjuvant disease = Dl;
urinary bladder cancer = dL; immunoglobulin g2 deficiency = dl; henoch-schoenlein purpura = dM; primary antiphospholipid
syndrome = dm; diabetic foot ulcer = Dm; single organ dysfunction = DM; meningococcemia = DN; esophageal candidiasis =
Dn; dermatitis = dN; extramedullary hematopoiesis = dn; neurogenic hypertension = do; endocrine gland cancer = Do;
pneumocystosis = dO; malignant state = DO; familial thrombotic thrombocytopeniac purpura = DP; placental malformation =
dp; pulmonary function = Dp; endometriosis = dP; disease of metabolism = dq; inclusion body myositis = Dq; familial
hyperlipidemia = dQ; arterionephrosclerosis = DQ; microangiopathyic hemolytic anemia = DR; myotonic dystrophy type 1 =
dR; genetic disease = dr; congenital hypoplasia = Dr; bronchiolitis, viral = Ds; influenza = ds; anthrax disease =
DS; creutzfeldt-jakob disease = dS; prion disease = dt; pulmonary alveolar proteinosis = dT; respiratory syncytial virus
bronchiolitis = Dt; thrombotic thrombocytopeniac purpura = DT; asthma chronic = Du; thrombocytopenia = DU; lipid storage
disease = du; cutaneous mastocytosis = dU; malaria = dV; congenital disorder of glycosylation = dv; nasal allergy = Dv;
disseminated intravascular coagulation = DV; glaucoma = dw; membranoproliferative glomerulonephritis = DW; aplastic anemia =
dW; respiratory syncytial virus infectious disease = Dw; cone-rod dystrophy 9 = Dx; purpura = DX; peripheral primitive
neuroectodermal tumor = dx; blood coagulation disease = dX; kidney disease = dy; carotid artery disease = dY; skin melanoma =
Dy; autoimmune thrombocytopeniac purpura = DY; intracranial arteriosclerosis = dZ; breast fibroadenoma = Dz; bone marrow
cancer = dz; ehlers-danlos syndrome, type vii, autosomal recessive = DZ; cardiovascular system disease = e; acute kidney
injury = E; arteriosclerosis = ea; lymphocytic choriomeningitis = EA; body fat distribution = Ea; malignant glioma =
eA; eclampsia = eb; weill-marchesani-like syndrome = Eb; hepatitis d = EB; chronic granulomatous disease = eB; index
myopia = Ec; syringomyelia = eC; familial hypercholesterolemia = ec; reticulate acropigmentation of kitamura = EC; embryonal
carcinoma = eD; hypolipoproteinemia = ed; knobloch syndrome = Ed; mental retardation, autosomal recessive 36 = ED; spinocerebellar
ataxia, autosomal recessive 9 = EE; tangier disease = ee; atopic dermatitis = eE; microcornea, myopiac chorioretinal
atrophy, and telecanthus = Ee; sexual dysfunctions, psychological = Ef; osteosarcoma = eF; norum disease = ef; coenzyme
q10 deficiency, primary, 1 = EF; plasmodium falciparum malaria = eg; coenzyme q10 deficiency = EG; psychotic symptom =
Eg; fibrosarcoma = eG; ehlers-danlos syndrome = Eh; cerebral malaria = eh; bone osteosarcoma = eH; ataxiac = EH; post-traumatic
stress disorder = Ei; nephrotic syndrome, type 9 = EI; intracranial thrombosis = eI; metabolic syndrome x = ei; pancreas
adenocarcinoma = eJ; cholesterol ester storage disease = ej; pseudohypoparathyroidism, type ia = EJ; laryngeal squamous
cell carcinoma = Ej; lumbar disc prolapse = Ek; hypercalciuria, absorptive, 2 = EK; cerebral infarction = eK; niemann-pick
disease = ek; corneal neovascularization = El; smith-lemli-opitz syndrome = el; bladder carcinoma = eL; pseudohypoparathyroidism =
EL; non-small cell lung carcinoma = eM; geleophysic dysplasia 1 = Em; thoracic cancer = em; severe chronic obstructive
pulmonary disease = EM; acromicric dysplasia = En; olfaction disorders = EN; squamous cell carcinoma = en; coronary stenosis =
eN; mitral valve stenosis = eo; hand deformities, congenital = Eo; sebaceous adenocarcinoma = eO; dyskinesia, familial,
with facial myokymia = EO; geleophysic dysplasia = Ep; ehrlich tumor carcinoma = eP; menkes disease = ep; cystic kidne =
EP; ectopia lentis 2, isolated, autosomal recessive = Eq; electrocardiogram (ekg, ecg) = EQ; arteriosclerotic cardiovascular
disease = eq; leber hereditary optic neuropathy = eQ; ectopia lentis isolated = Er; chronic kidney failure = eR; nephroblastoma =
er; alpha-thalassemia = ER; ectopia lentis with ectopia of pupil = Es; urinary bladder, overactive = ES; female breast
carcinoma = eS; kidney cancer = es; intestinal volvulus = eT; chronic myeloproliferative disease = et; miosis = ET; ectopia
lentis, isolated, autosomal recessive = Et; brain ischemia = eu; sezary's disease = eU; pancreatitis, acute necrotizing =
EU; simple ectopia lentis = Eu; colon adenocarcinoma = ev; nfl microdeletion syndrome = Ev; facial nerve injuries =
EV; hodgkin's lymphoma = eV; neurofibromatosis = Ew; bloom syndrome = ew; partial trisomy = EW; herpes simplex = eW;
glycogen storage disease i = ex; myeloma kidney = EX; aicardi-goutieres syndrome = Ex; hodgkin's granuloma = eX; wilson
disease = eY; aicardi-goutieres syndrome 6 = Ey; cystitis = EY; iga glomerulonephritis = ey; sudden infant death syndrome =
EZ; aicardi goutieres syndrome = Ez; antiphospholipid syndrome = ez; systemic lupus erythematosus = eZ; down syndrome =
f; hepatitis c, chronic = F; rds - infants = fA; polymicrogyria, bilateral perisylvian, autosomal recessive = FA; cerebral
amyloid angiopathy = fa; symptoms of stress = Fa; pulmonary interstitial glycogenosis = fB; prostate adenocarcinoma =
Fb; congenital anomaly of brain = FB; gastrointestinal stromal tumor = fb; fatty liver disease = fc; oligodendroglioma =
Fc; cobblestone complex = FC; persistent pulmonary hypertension = fC; xerocytosis, hereditary = Fd; lissencephaly =
FD; multiple myeloma = fd; pneumonia = fD; neonatal respiratory failure = fE; hypernatremia = Fe; inflammatory bowel
disease = fe; secondary hyperparathyroidis = FE; gastroschisis = Ff; scoliosis, idiopathyic, susceptibility to, 1 = FF;
tongue squamous cell carcinoma = ff; adult respiratory distress syndrome = fF; simpson-golabi-behmel syndrome = fg; pulmonary
fibrosis = fG; adolescent idiopathyic scoliosis = FG; conn's syndrome = Fg; meniere's disease = Fh; persistent fetal
circulation syndrome = fH; gastriti = FH; paroxysmal nocturnal hemoglobinuria = fh; familial combined hyperlipidemia =
Fi; behavior disorder = FI; chylomicron retention disease = fi; desquamative interstitial pneumonia = fI; allergic
contact dermatitis = fJ; hyperaldosteronism = Fj; lamellar ichthyosis, type 2 = fj; autosomal dominant lateral temporal
lobe epilepsy = FJ; usher syndrome, type 2c = FK; chondroma = Fk; harlequin type ichthyosis = fk; alzheimer's disease
with late onset = fK; febrile convulsions, familial, 4 = FL; endolymphatic hydrop = Fl; lupus erythematosus = fL; nevus
elasticus = fl; recovery of function = fm; spherocytosis, type 1 = Fm; usher syndrome, type iic, gpr98/pdzd7, digenic =
FM; b-cell lymphoma = fM; congenital ichthyosisform erythroderma = fn; hand skill, relative = Fn; decompression sickness =
FN; sjogren's syndrome = fN; arthritis, experimental = fO; benzodiazepines causing adverse effects in therapeutic use =
FO; pseudoxanthoma elasticum = fo; attention deficit hyperactivity disorder = Fo; cednik syndrome = fp; perineurioma =
Fp; poisoning by benzodiazepine-based tranquilizer = FP; renal insufficiency = fP; usher syndrome type 2 = FQ; encounter
for body mass index [bmi] = fq; liver cirrhosis, experimental = fQ; biliary atresia = Fq; afebrile seizure = FR; alpers
syndrome = Fr; infection = fR; renal cell carcinoma = fr; audiogenic seizure = FS; endolymphatic hydrops = Fs; drug hypersensitivity =
fS; low grade glioma = fs; balo concentric sclerosis = Ft; helicobacter infections = fT; substance addiction = FT;
lung small cell carcinoma = ft; xanthoma tendinosum = FU; neuropathy = fu; myeloid sarcoma = Fu; renal insufficiency,
chronic = fU; enzyme inhibition = FV; pulmonary large cell neuroendocrine carcinoma = Fv; surfactant metabolism dysfunction,
pulmonary, 3 = fv; drug-related side effects and adverse reactions = fV; respiratory tract diseases = fw; nonmedullary
thyroid carcinoma, with or without cell oxyphilia = fW; prenatal exposure delayed effects = FW; emery-dreifuss muscular
dystrophy = Fw; alcohol withdrawal syndrome = FX; surfactant dysfunction = fx; multicystic renal dysplasia, bilateral =
fX; carcinoma, anaplastic = Fx; inflammatory bowel disease 13 = fY; alcohol-related disorders = FY; polymicrogyria,
bilateral frontoparietal = Fy; interstitial fibrosis = fy; neoplasmtic processes = fZ; chronic interstitial lung disease =
fz; cocarcinogenesis = FZ; ehlers-danlos syndrome, type iv, autosomal dominant = Fz; prostatic hyperplasia = G; adenocarcinoma =
g; oropharynx cancer = GA; alcohol-induced disorders = Ga; hiv-infection/aids = gA; postoperative complications = ga;
parkinson disease, late-onset = GB; abnormal renal function = gB; femur head necrosis = gb; heavy drinking = Gb; birth
defects = Gc; drug usage = gC; colchicine resistance = gc; multiple system atrophy = GC; colorectal cancer metastatic =
gd; hippocampal sclerosis = gD; succinic semialdehyde dehydrogenase deficiency = GD; alcohol use disorder = Gd; monocytic
leukemia = gE; sensory discomfort = Ge; disease of metabolis = GE; chronic ulcerative colitis = ge; depressive symptom =
gf; schizophrenia and related disorders = gF; alcohol affecting fetus or newborn via placenta or breast milk = Gf;
choroidal neovascularization = GF; associated symptom = Gg; hiv-associated lipodystrophy syndrome = GG; steroid-sensitive
nephrotic syndrome = gG; kidney replaced by transplant = gg; androgen independent prostate cancer = gh; prostatic hypertrophy =
gH; adiponectin, serum level of, quantitative trait locus 3 = GH; drunk driving = Gh; withdrawal sign or symptom =
gI; hazardous drinking = Gi; adiponectin, serum level of, quantitative trait locus i = GI; steroid-resistant nephrotic
syndrome = gi; ovarian cancer stage = gJ; pancreatitis, calcific = Gj; glucocorticoid receptor deficiency = gj; adiponectin,
serum level of, quantitative trait locus 2 = GJ; obesity, visceral = GK; acute myeloid leukemia arising from previous
myelodysplastic syndrome = gK; cirrhosis = gk; alcohol abuse or dependencye = Gk; cns depression = gL; laryngeal benign
neoplasm = Gl; psychosis = gl; hypoadiponectinemia = GL; wernicke-korsakoff syndrome = Gm; drug withdrawal syndrome =
gM; cytogenetically normal acute myeloid leukemia = gm; headache disorders = GM; larynx cancer = Gn; insulin resistance
in diabetes = GN; retroviridae infections = gn; pediatric aids = gN; sexual dysfunction = gO; atrophy of testis = Go;
carotid artery plaque = GO; clear-cell metastatic renal cell carcinoma = go; conduct disorder = Gp; philadelphia chromosome
positive chronic myelogenous leukemia = gp; diabetes mellitus risk = GP; testicular carcinoma = gP; cardiac event = gq;
macrocytic anemia = Gq; sarcoma, metastatic = gQ; microalbuminuric diabetic nephropathy = GQ; lipodystrophy = GR; postoperative
nausea and vomiting = gr; alcoholic neuropathy = Gr; postoperative nausea = gR; glucose metabolism disease = GS; breast
cancer, lobular = gs; alcohol abuse = Gs; sphenoid wing meningioma = gS; pouchitis = gt; hypothyroidism = gT; essential
tremor = Gt; arteriosclerosis obliterans = GT; alcohol dependencye = Gu; erythroblastosis = GU; male infertility = gU;
absence of pain sensation = gu; nicotine dependencye = Gv; menopause present = gv; neutropenia = gV; adiponectin deficiency =
GV; skin toxicity = gw; urinary system benign neoplasm = gW; hypermethioninemia due to adenosine kinase deficiency =
GW; pharynx cancer = Gw; gastrointestinal system cancer = gX; cocaine dependency = Gx; visceral steatosis, congenital =
GX; early pregnancy = gx; pancolitis = gy; streptococcal lymphadenitis of swine = Gy; tooth eruption = GY; heart septal
defect = gY; heart--cancer = gz; recurrent seizures = GZ; heart disease = gZ; sjogren-larsson syndrome = Gz; human immunodeficiency
virus infectious disease = h; plaque, amyloid = H; vitreoretinopathy, neovascular inflammatory = Ha; vogt-koyanagi-harada
disease = HA; epilepsy syndrome = ha; agranulocytosis = hA; gallbladder cancer = hB; brain disease = hb; renal fibrosis =
Hb; carney complex = HB; nerve degeneration = HC; bilirubin metabolic disorder = hC; glomerulonephritis = hc; gastrointestinal
tract vascular insufficiency = Hc; epilepsy, post-traumatic = HD; dysglycemia = Hd; thyroid carcinoma = hd; graves' disease =
hD; hepatitis a = hE; myopia 2 = HE; disease of mental healtyh = he; bipolar ii disorder = He; hematologic cancer =
hf; syndrome of infant of diabetic mother = Hf; tardive dyskinesia = HF; acute leukemia = hF; leukopenia = hG; stricture
of artery = Hg; idiopathyic generalized epilepsy = hg; chronic progressive chorea = HG; abnormal involuntary movement =
HH; encephalyitis = hh; behcet's disease = hH; medullary tumors = Hh; osteonecrosis = hi; renal interstitial fibrosis =
HI; swelling of limb = Hi; tuberous sclerosis = hI; lipoma = hj; macular fibrosis = Hj; phobic disorder = HJ; cholestasis =
hJ; lesch-nyhan syndrome = HK; asphyxia neonatorum = Hk; skin benign neoplasm = hk; addison's disease = hK; liver disease =
hl; lingual-facial-buccal dyskinesia = HL; primary hyperparathyroidism = Hl; endometrial cancer = hL; acute myocardial
infarction = hm; hypercholesterolemia, autosomal recessive = HM; myasthenia gravis = hM; diabetic ketoacidosis = Hm;
sclerosisng cholangitis = hN; teratocarcinoma = Hn; lymphoblastic leukemia = hn; childhood absence epilepsy = HN; acute
retinal necrosis syndrome = Ho; congenital long qt syndrome = HO; fabry disease = hO; prolymphocytic leukemia = ho; malignant
peripheral nerve sheath tumor = hP; pilocytic astrocytoma = Hp; celiac disease = hp; sciatica = HP; x-linked ichthyosis =
hQ; anxiety disorder = hq; low tension glaucoma = HQ; choroid plexus carcinoma = Hq; varicose veins = Hr; hyperthyroidism =
hr; pituitary cancer = hR; bladder neck obstruction = HR; lymphadenitis = hs; malignant mesothelioma = hS; proliferative
vitreoretinopathy = Hs; alport syndrome, x-linked = HS; esophageal cancer = ht; hypokalemic periodic paralysis, type
1 = HT; ileitis = Ht; peritoneal carcinoma = hT; adrenal gland cancer = Hu; esophageal carcinoma = hu; cannabis dependencye =
hU; night blindness, congenital stationary, type 2a = HU; hypoxia-ischemia, brain = Hv; malignant fibroxanthoma = hV;
ehlers-danlos syndrome type 2 = HV; vasculitis = hv; kniest dysplasia = HW; uterine cancer = hw; optic nerve injuries =
Hw; leiomyosarcoma = hW; gastroparesis = hx; generalized epilepsy with febrile seizures plus, type 2 = HX; tauopathy =
Hx; hepatitis b = hX; head and neck cancer = hy; kernicterus = hY; hearing loss, bilateral = Hy; hyperpigmentation,
familial progressive = HY; tn syndrome = HZ; thyroid gland disease = hz; osteosclerosis = Hz; opiate dependencye = hZ;
exanthema = i; aural atresia, congenital = I; osteogenesis imperfecta, type la = IA; papillary serous adenocarcinoma =
ia; hemiplegiac migraine, familial type 1 = Ia; renal carcinoma = iA; mucoepidermaoid carcinoma = iB; spinocerebellar
ataxia 6 = Ib; deficiency of trypsin = IB; benign mesothelioma = ib; selective iga deficiency disease = IC; benign ependymoma =
iC; spondyloepiphyseal dysplasia tarda, x-linked = Ic; spleen cancer = ic; transitional cell carcinoma = id; varicocele =
ID; porencephaly = Id; juvenile myoclonic epilepsy = iD; pelvic organ prolapse = Ie; bronchiolo-alveolar adenocarcinoma =
iE; mucopolysaccharidosis iii = IE; liver sarcoma = ie; cervical cancer = if; extraosseous ewings sarcoma-primitive
neuroepithelial tumor = iF; muscle eye brain disease = If; stiff-person syndrome = IF; thyrotoxic periodic paralysis =
Ig; alcoholic pancreatitis = iG; pulmonary tuberculosis = ig; skin sarcoidosis = IG; schizoaffective disorder = iH;
hypochondrogenesis = Ih; familial mediaterranean fever = ih; excessive tearing = IH; osteopeniac nonfracture syndrome =
Ii; premature ovarian failure = iI; balkan nephropathy = ii; interstitial cystitis = II; hypokalemic periodic paralysis =
IJ; central nervous system lymphoma = ij; synovial sarcoma = iJ; urinary incontinence, stress = Ij; head and neck squamous
cell carcinoma = iK; aids-related complex = Ik; chordoma = ik; lipoid proteinosis = IK; temporal lobe epilepsy = il;
type i ehlers-danlos syndrome = IL; adult acute lymphocytic leukemia = iL; hyperexplexia = Il; acute t cell leukemia =
iM; diffuse scleroderma = IM; breast adenocarcinoma = im; epileptic encephalyopathy = Im; pleomorphic liposarcoma =
IN; chronic interstitial cystitis = IN; invasive lobular carcinoma = in; chondrodysplasia = In; anti-neutrophyil cytoplasmic
antibody-associated vasculitis = Io; hepatoblastoma = iO; scleredema adultorum = IO; gallbladder carcinoma = io; meningioma =
ip; acquired porencephaly = Ip; cutis laxa = IP; uveal melanoma = iP; congenital scoliosis = Iq; acquired immunodeficiency
syndrome = iQ; toxic encephalyopathy = iq; granuloma annulare = IQ; age-related cognitive decline = Ir; ovary adenocarcinoma =
ir; liver carcinoma = iR; scleroderma = IR; asymptomatic bacteriuria = Is; parathyroid adenoma = iS; pyruvate carboxylase
deficiency disease = is; leiomyomatosis = IS; myeloid leukemia = iT; chronic inflammatory disorder = It; esophagus squamous
cell carcinoma = it; prolactinoma = IT; lung adenocarcinoma = iu; spastic = Iu; oral submucous fibrosis = IU; chronic
myeloid leukemia = iU; homozygous alpha thalassemia = Iv; ulcerative colitis = iV; dermatitis herpetiformis = IV; adrenocortical
carcinoma = iv; malignant hyperthermia = IW; drug habituation = Iw; lymphosarcoma = iW; adrenal cortex cancer = iw; uterine
corpus sarcoma = ix; anorexia nervosa = iX; eosinophilia-myalgia syndrome = IX; lymphocytic infiltration = Ix; coronary
restenosis = iy; contracture of joint = Iy; megakaryocytic leukemia = iY; immunoglobulin alpha deficiency = IY; heavy
chain disease = IZ; arterial aneurysm = Iz; phototoxic dermatitis = iz; acute monocytic leukemia = iZ; diabetes = J;
coronary artery disease = j; nasopharynx carcinoma = ja; acute asthma = JA; acth-independent macronodular adrenal hyperplasia =
Ja; ornithine carbamoyltransferase deficiency = jA; cholestasis, progressive familial intrahepatic 3 = jB; cannabis
abuse = jb; asthma adult onset = JB; visceral pain = Jb; neonatal abstinence syndrome = jc; substance use = Jc; myocardial
failure = JC; scott syndrome = jC; rheumatic mitral stenosis = JD; inattention = Jd; cholestasis, intrahepatic, of pregnancy
3 = jD; heroin dependencye = jd; non-cardiogenic pulmonary edema = JE; adult t-cell leukemia = je; motion sickness =
Je; hepatitis b, chronic = jE; mild persistent asthma = JF; cholesterol gallstones = jF; medulloblastoma = jf; vasoconstriction =
Jf; gamma chain deficiency = jg; nocturnal cough = JG; advanced cirrhosis = jG; neoplasm of uncertain behavior of mediastinum =
Jg; bacterial meningitis = JH; diarrheal disorder = jH; functional gastrointestinal disorder = Jh; lymphoma = jh; portal
fibrosis = jI; gastric adenocarcinoma = JI; colitis = ji; constipation = Ji; hydrophthalmos = JJ; acute promyelocytic
leukemia = j; stage, chronic lymphocytic leukemia = jJ; cocaine abuse = Jj; afibrinogenemia congenital = jk; trachoma =
JK; cholestasis chronic = jK; resting heart rate = Jk; obstructive jaundice = jL; acanthosis nigricans = JL; social
fear = Jl; gallbladder disease 1 = jl; rhinitis = JM; hemangioma = Jm; cholestasis, progressive familial intrahepatic
2 = jm; cholangitis = jM; cholera = jN; capillary hemangioma = Jn; cholestasis, benign recurrent intrahepatic, 2 = jn;
status asthmaticus = JN; cholecystitis = jO; endotoxemia = jo; 5q-syndrome = Jo; inguinal hernia = JO; liver neoplasms,
experimental = jp; respiratory hypersensitivity = Jp; calcification of joints and arteries = jP; lipodystrophy, congenital
generalized, type 2 = JP; precocious pubarche = JQ; vestibulocochlear nerve injuries = Jq; intrahepatic cholestasis of
pregnancy = jq; brooke-spiegler syndrome = jQ; psoriasis vulgaris = Jr; lipoatrophyic diabetes = JR; hot spots = jr;
microphthalmia, isolated, with coloboma 7 = jR; chromosome 5, trisomy 5q = Js; dyschromatosis universalis hereditaria
3 = jS; cholemia = js; multiple symmetrical lipomatosis = JS; psychological distress = Jt; embryo loss = JT; immune suppression =
jT; chronic hepatic failure = jt; cardiac function, impaired = JU; intestinal disease = ju; blood group, langereis
system = jU; parasitic diseases = Ju; asthma night-time symptoms = Jv; placenta disease = jv; hypertensive left ventricular
hypertrophy = JV; creutzfeldt-jakob disease, sporadic = jV; acromegaly = JW; intrahepatic cholestasis = jw; chiari malformation
type i = jW; peak expiratory flow rate = Jw; hepatitis c = jx; whim syndrome = JX; variant creutzfeldt-jakob disease =
jX; allergic sensitization = Jx; hyperinsulinism = jy; pregnancy related = Jy; adenylosuccinate lyase deficiency =
JY; creutzfeldt-jakob disease, familial = jY; cholangiocarcinoma = jz; acute type b viral hepatitis = JZ; parasitemia =
jZ; signs and symptoms, respiratory = Jz; stress (psychology) = K; precursor cell lymphoblastic leukemia-lymphoma =
k; oculomotor apraxia = KA; sideroblastic anemia = kA; argininosuccinic aciduria = Ka; caries = ka; chronic plaque-like
oral candidiasis = kb; eczema herpeticu = Kb; hereditary ataxia = KB; autosomal dominant cerebellar ataxia = kB; mitochondrial
metabolism disease = kC; memories = Kc; dream disorder = kc; intelligence quantitative trait locus 1 = KC; thrombocytosis =
kD; acute respiratory infections = kd; chronic infectious disease = Kd; unable to balance = KD; finnish lethal neonatal
metabolic syndrome = kE; pathyologic nystagmus = KE; central nervous system disease = ke; acute infectious disease =
Ke; mutyh-associated polyposis = Kf; spinal muscular atrophy = kf; metaphyseal chondrodysplasia = kF; cogan syndrome =
KF; increased drug resistance = kG; mucopolysaccharidosis = kg; spindle cell sarcoma = Kg; ptosis = KG; t-cell leukemia =
kH; spinocerebellar ataxia, autosomal recessive 1 = KH; spinocerebellar ataxia 1 = Kh; chondrosarcoma = kh; arteriovenous
malformation = kI; nephrosis, congenital = KI; fraxe = Ki; dental caries = ki; microphthalmia = kj; spinal cancer = kJ;
alpha-fetoprotein deficiency = KJ; spinobulbar atrophy = Kj; alpha-fetoprotein, hereditary persistence of = KK; mental
handicap = Kk; dubin-johnson syndrome = kK; tetanus = kk; sex chromosome aberrations = KL; fraxa = Kl; polycystic ovary
syndrome = kl; alcoholic liver cirrhosis = kL; congenital disorders = Km; cartilage-hair hypoplasia = kM; liver regeneration =
KM; limb-girdle muscular dystrophy = km; duodenitis = kN; coloboma = kn; borderline mental retardation (i.q. 70-85) =
Kn; genetic syndrome = KN; speech delay = Ko; lichen planus = kO; aniridia = ko; multiple tumors = KO; triple negative
breast neoplasms = kP; moderate mental retardation (i.q. 35-49) = Kp; uv-sensitive syndrome = KP; pancreatic ductal adenocarcinoma =
kp; developmentally delayed = Kq; renal tubular disorder = kQ; gerstmann-straussler-scheinker syndrome = kq; nonseminomatous
germ cell tumor = KQ; language delay = Kr; telangiectatic osteogenic sarcoma = kr; apocrine gland secretion, variation
in = kR; tumor thrombus = KR; promyelocytic leukemia = kS; mental deficiency = Ks; actinic keratosis = ks; autoimmune
liver disease = KS; atrophyic gastritis = kt; restenosis = kT; foot tapping = Kt; benign neoplasm of liver = KT; fatal
familial insomnia = ku; juvenile dermatomyositis = kU; agnosia = KU; fragile x syndrome = Ku; intestinal cancer = KV;
pseudomonas aeruginosa infection = kV; costello syndrome = kv; machado-joseph disease = Kv; occipital lobe neoplasm =
Kw; bromhidrosis = kW; oculocerebrorenal syndrome = KW; dyschromatosis universalis hereditaria = kw; anemia, sideroblastic
spinocerebellar ataxia = kx; abdominal sepsis = kX; cutaneous porphyria = KX; kennedy's disease = Kx; cerebellar ataxia =
ky; endodermal sinus tumor = KY; spinocerebellar ataxia 28 = Ky; bacterial keratitis = KY; gonadal disease = KZ; refractory
anemia (clinical) = kz; spastic ataxia 5, autosomal recessive = Kz; pneumonia due to klebsiella pneumoniae = kZ; senile
plaque = L; cholelithiasis = l; corneal disease = la; cockayne syndrome = La; thromboangiitis obliterans = LA; right
ventricular failure = lA; substance-related disorder = lB; idiopathyic pulmonary fibrosis = LB; myositis = lb; germ cell
and embryonal cancer = Lb; sweat gland disease = lc; denys-drash syndrome = Lc; limb ischemia = LC; pulmonary hypertension =
lC; klippel-trenaunay syndrome = LD; spondyloarthropathy = ld; primary pulmonary hypertension = lD; testicular germ
cell cancer = Ld; keratitis = le; platelet storage pool deficiency = lE; dysgerminoma = Le; cataract and cardiomyopathy =
LE; testicular malignant germ cell cancer = Lf; cataract 38 = LF; hermansky-pudlak syndrome = lF; kawasaki disease =
lf; reperfusion injury = lG; latent tuberculosis = LG; ganglioglioma = Lg; papilloma = lg; active tuberculosis = LH;
malignant teratoma = Lh; childhood type dermatomyositis = lh; bone destruction = lH; pallister-hall syndrome = LI; ischemiac
cardiomyopathy = lI; pancreatic acinar cell adenocarcinoma = Li; skin atrophy = li; primary angle-closure glaucoma =
lJ; toxic hepatitis = lj; autoimmune thyroiditis = Lj; buruli ulcer disease = LJ; umbilical hernia = Lk; arterial calcification
of infancy = lK; neuronopathy, distal hereditary motor, type v = LK; salmonella infections = lk; omphalocel = Ll; lipodystrophy,
partial, acquired = LL; delayed renal graft function = ll; arterial calcification, generalized, of infancy, 2 = lL; multisystem
disorder = lM; lipodystrophy, congenital generalized, type 1 = LM; severe diarrhea = lm; hypoparathyroidism familial
isolated = Lm; spastic paraplegia 17 = LN; metabolic disturbance = lN; bone marrow toxicity = ln; androgenetic alopecia =
Ln; mendelian disorders = lO; silver-russell syndrome = LO; viral infectious disease = lo; congenital, hereditary,
and neonatal diseases and abnormalities = Lo; tethered spinal cord syndrome = lp; retinal toxicity = lP; storage disease =
Lp; rhizomelic chondrodysplasia punctata, type 3 = LP; constrictive pericarditis = lq; lysosomal storage disease =
Lq; skin disease = lQ; sarcoma, experimental = LQ; focal epilepsy = lr; retinal disease = lR; aspartylglucosaminuri =
Lr; rhizomelic chondrodysplasia punctata = LR; calcinosis = lS; extrahepatic cholestasis = ls; neurilemmoma = Ls; hormone
refractory prostate cancer = LS; staphylococcal toxic shock syndrome = LT; bile duct cancer = lt; angioid streaks = lT;
corneal dystrophy, fuchs endothelial, 8 = Lt; bullous impetigo = LU; fuchs' endothelial dystrophy = Lu; myelomaeningocel =
lU; clear cell adenocarcinoma = lu; focal nodular hyperplasia = lv; hyperinsulinemiac hypoglycemia, familial, 6 = lV;
pancreatic intraepithelial neoplasmia (panin) = LV; hyperoxia = Lv; bone peripheral neuroepithelioma = lw; peritoneal
fibrosis = Lw; virchow's node = LW; diabetes mellitus, transient neonatal, 1 = lW; pulmonary hypertension, primary, 1 =
lx; hypoglycemia, leucine-induced = lX; toxic shock syndrome = LX; lung injury = Lx; barrett's adenocarcinoma = LY;
pulmonary arterial hypertension = ly; aortic calcification = Ly; diabetes mellitus, transient neonatal, 2 = lY; barrett's
esophagus = LZ; aa amyloidosis = Lz; secretory diarrhea = lz; fanconi-bickel syndrome = lZ; ischemiac cerebrovascular
accident = M; ovarian carcinoma = m; color vision defects = mA; hyperinsulinemiac hypoglycemia, familial, 1 = ma; glucocorticoid-remediable
aldosteronism = MA; borderline ovarian tumorur = Ma; hyperinsulinemiac hypoglycemia, familial, 2 = mb; primary hyperoxaluria =
MB; intrahepatic cholangiocarcinoma = Mb; hypoadrenocorticism, familial = mB; congenital myasthenic syndrome ib = Mc;
usher syndrome, type lc = mc; lupus vulgaris = mC; peutz-jeghers syndrome = MC; inflammatory breast carcinoma = MD; cardiac
defects = md; myasthenic syndrome, congenital, with pre-and postsynaptic defects = Md; adrenomyeloneuropathy = mD; peroxisomal
disease = mE; congenital bilateral aplasia of vas deferens = Me; goodpasture syndrome = ME; developmental delay, epilepsy,
and neonatal diabetes = me; aids defining illness = Mf; liddle syndrome = MF; demyelinating disease = mF; glucose intolerance =
mf; kartagener syndrome = MG; tetralogy of fallot = Mg; sensorineural hearing loss = mG; gestational diabetes = mg;
spinal cord disease = mH; neonatal diabetes mellitus = mh; crigler najjar syndrome, type 2 = Mh; radiation injuries,
experimental = MH; prehypertension = MI; hyperinsulinemiac hypoglycemia = mi; endocrine system disease = mI; aortic rupture =
Mi; acute stroke = MJ; neointima = Mj; carpal tunnel syndrome = mJ; insulinoma = mj; brain edema = mk; rheumatic disease
of heart valve = MK; acquired long qt syndrome = Mk; autoimmune lymphoproliferative syndrome = mK; female breast hyperplasia =
ML; iga nephropathy, progression to renal failure in, susceptibility to = Ml; beckwith-wiedemann syndrome = ml; zellweger
syndrome = mL; secondary hypertension = MM; methylmalonic aciduria and homocystinuria, cblj type = mM; hypertriglyceridemic
waist = Mm; cantu syndrome = mm; vitamin b 12 deficiency = mN; adrenal rest tumor = MN; renal anemia = Mn; cardiomyopathy,
dilated, lo = mn; inherited metabolic disorder = mO; lesion of brain = Mo; primary idiopathyic dilated cardiomyopathy =
mo; cystadenoma = MO; atrial fibrillation, familial, 12 = mp; thrombotic stroke = Mp; placental insufficiency = MP;
graves ophthalmopathy = mP; intracranial embolism = MQ; myocardial infarction, susceptibility to, 1 (finding) = mq; bleeding
ulcer = Mq; hypersensitivity reaction type i disease = mQ; ankylosing spondylitis = mR; aging, premature = MR; fibrillation =
mr; low-renin essential hypertension = Mr; renal dysplasia = MS; early repolarization associated with ventricular fibrillation =
ms; major affective disorder 1 = mS; renin induced hypertension = Ms; atresia = MT; major affective disorder 5 = mT;
prinzmetal's variant angina = mt; multi vessel coronary artery disease = Mt; pineoblastoma = MU; long qt syndrome = mu;
mesial temporal sclerosis = Mu; major affective disorder 8 = mU; major affective disorder 9 = mV; hypertrichosis = mv;
renin-dependent hypertension = Mv; subacute bacterial endocarditis = MV; hypertensive heart failure = Mw; coronary artery
vasospasm = mw; familial retinoblastoma = MW; major affective disorder 7 = mW; homocystinuria = MX; dilated cardiomyopathy =
mx; major affective disorder 4 = mX; chronic hypotension = Mx; sciatic neuropathy = My; major affective disorder 2 =
mY; congenital disorder of glycosylation type la = MY; imerslund-grasbeck syndrome = my; major affective disorder 6 =
mZ; hypermethioninemia with deficiency of s-adenosylhomocysteine hydrolase = MZ; prediabetes syndrome = Mz; intrinsic
factor deficiency = mz; recurrent malignant neoplasm = n; alpha-1-antitrypsin deficiency, autosomal recessive = N; splenic
disease = NA; ‘as if’ personality = na; intellectual disability = nA; cleft palate = Na; thyroid cancer = nB; ureteral
benign neoplasm = NB; macular degeneration = nb; pyridoxine deficiency anemia = Nb; chronobiology disease = NC; retinitis
pigmentosa = nC; cleft lip = Nc; creatine deficiency, x-linked = nc; gout = nD; mental retardation, x-linked 1 = nd;
joubert syndrome 3 = Nd; oligospermia = ND; human t-cell lymphotropic virus 1 infection = Ne; mucinous adenocarcinoma =
NE; neoplasm recurrence, local = ne; erythropoietic protoporphyria = nE; peanut allergic reaction = NF; squamous cell
neoplasm = nF; mental retardation, x-linked = nf; tumors in animals = Nf; cerebellar disease = Ng; congenital nystagmus =
NG; folic acid deficiency = ng; neuroendocrine carcinoma = nG; photoparoxysmal response 1 = nh; leber congenital amaurosis =
Nh; hyperuricemia = nH; uterine disease = NH; placental choriocarcinoma = nI; barakat syndrome = NI; gastroenteritis =
Ni; renal cell carcinoma, nonpapillary = ni; choriocarcinoma = nJ; periodic syndrome = NJ; cystic kidney = Nj; blood
group, junior system = nj; autosomal recessive retinitis pigmentosa = nk; dysembryoplastic neuroepithelial tumor = nK;
parvoviridae infections = NK; hepatitis e = Nk; photosensitization = nl; chloracne = Nl; gallbladder adenocarcinoma =
nL; primary infection nos = NL; malignant biphasic mesothelioma = Nm; infection induced = NM; endocarditis, bacterial =
nm; papillary thyroid carcinoma = nM; nevus, epidermal = Nn; parkinson disease, familial, type 1 = NN; melioidosis =
nN; serum uric acid = nn; pancreatic cancer, adult = No; dental white spot = NO; malignant pleural mesothelioma = nO;
weakness = no; neuromyelitis optica = nP; multiple malignancy = np; bone resorption = Np; asbestosis = NP; scrub typhus =
NQ; gout tophaceous = nq; ischemiac colitis = nQ; eosinophilic disorder = Nq; tuberculosis of intestines = nr; disorder
characterized by eosinophilia = Nr; rectal tumors = NR; sitosterolemia = nR; insulin resistance syndrome = nS; transient
ischemia = ns; rectal neoplasm = NS; penias agenisi = Ns; diarrhea due to drug = nt; polygenic hypercholesterolemia =
nT; rectum cancer = NT; sleep disorder = Nt; uroporphyrinuria = Nu; congenital dyserythropoietic anemia, type ii = NU;
lipid metabolism disorder = nU; chronic tophaceous gout = nu; terminal insomnia = Nv; thyroid medullary carcinoma = nv;
immunodeficiency with hyper-igm, type 2 = NV; xanthomatosis = nV; asthma attack = Nw; gallbladder disease 4 = nW; retinal
degeneration = nw; minicore myopathy with external ophthalmoplegia = NW; syndrome = nX; chronic nonspecific lung disease =
Nx; fundus dystrophy = nx; hereditary autoinflammatory diseases = NX; biliary tract cancer = nY; silicosis = Ny; periodic
fever = NY; pleural cancer = ny; bladder calculus = Nz; light fixation seizure syndrome = NZ; tongue cancer = nz; bile
duct carcinoma = nZ; embolism, paradoxical = O; juvenile rheumatoid arthritis = o; autosomal recessive hyper-igm syndrome =
Oa; acute undifferentiated leukemia = oA; pituitary adenoma, growth hormone-secreting = OA; polyneuropathy, hearing
loss, ataxia, retinitis pigmentosa, and cataract = oa; gigantism and acromegaly = OB; sialadenitis = Ob; polyneuropathy =
ob; leukaemia secondary = oB; usher syndrome = oc; b acute lymphoblastic leukemia with t(9 = oC; acute intermittent
porphyria = OC; selective ige deficiency disease = Oc; cII/sII = Od; pituitary-dependent cushing's disease = OD; coronary
aneurysm = od; t(9;22)(q34;q11) = oD; x-linked hyper igm syndrome = Oe; q11.2) = oE; pituitary carcinoma = OE; arterial
pressure = oe; hormone producing pituitary cancer = OF; immunoglobulin a deficiency 1 = of; ber-abl1 = oF; macroglobulinemia =
Of; triglyceride storage disease with impaired long-chain fatty acid oxidation = og; pre b-cell acute lymphoblastic
leukemia = oG; growth hormone secreting pituitary adenoma = OG; lymphoplasmacytic lymphoma = Og; acth-secreting pituitary
adenoma = OH; alcohol-induced disorders, nervous system = Oh; malignant disease = oH; protein deficiency = oh; neuritis,
autoimmune, experimental = Oi; bipolar i disorder = oi; leber congenital amaurosis 4 = OI; monocytosis = oI; vasculitic
neuropathy = Oj; myopathy = oj; ouabain resistance = oJ; retinitis pigmentosa, juvenile, aipli-related = OJ; substance
abuse = ok; granulocytosis = oK; meningoencephalyitis = Ok; retinal dystrophy, early onset severe = OK; motor neuron
disease = Ol; cone dystrophy = OL; multiple acyl-coa dehydrogenase deficiency = ol; leukaemia recurrent = oL; polyglandular
autoimmune syndrome type i = OM; metabolic syndrome = om; neuritis = Om; stage, chronic myelogenous leukemia = oM; gliosarcoma =
On; spastic syndrome = on; indolence = ON; autoimmune polyendocrinopathy syndrome, type 1 = ON; chlamydia trachomatis
infection = oo; bacterial infectious disease = oO; autoimmune polyendocrinopathy syndrome, type i, autosomal dominant =
OO; combined oxidative phosphorylation deficiency 6 = Oo; autoimmune polyendocrinopathy syndrome, type i, with reversible
metaphyseal dysplasia = OP; leiomyoma = op; myelodysplastic myeloproliferative cancer = oP; cowchock syndrome = Op; eye
disease = oQ; kashin-beck disease = oq; idiopathyic hypoparathyroidism = OQ; distal spinal muscular atrophy = Oq; hemophagocytic
lymphohistiocytosis, familial, 2 = or; rhabdoid cancer = Or; hepatitis immune = OR; cerebral palsy = oR; polyglandular
autoimmune syndrome type ii = OS; thymus lymphoma = oS; fanconi anemia, complementation group a = os; pneumococcal infections =
Os; recurrent herpes simplex = OT; polycythemia = oT; memory disorders = ot; vascular lesions = Ot; autoimmune primary
adrenal insufficiency = OU; respiratory failure = oU; neuroectodermal tumor = Ou; precursor b-cell lymphoblastic leukemia-lymphoma =
ou; autoimmune polyendocrine syndrome = OV; neoplasms, experimental = ov; primary immunodeficiency disease = oV; pelizaeus-merzbacher-like
disease, autosomal recessive, 2 = Ov; alopecia areata = OW; philadelphia chromosome positive = ow; porphyria, acute hepatic =
Ow; anemia = oW; pancytopenia = oX; hypoparathyroidism = OX; nematode infections = Ox; philadelphia chromosome-positive
acute lymphoblastic leukemia = ox; hypereosinophilic syndrome, idiopathyic = oy; hematopoietic system disease = oY; guillain-barre
syndrome = OY; pituitary adenoma, familial isolated = Oy; fanconi's anemia = oZ; chronic disease = oz; keratoconjunctivitis
sicca = OZ; familial isolated pituitary adenoma = Oz; mitochondrial cardiomyopathy = p; deep thrombophlebitis = P; epidermaodysplasia
verruciformis = pa; prostatic cancer, castration-resistant = PA; chronic mucocutaneous candidiasis = Pa; von willebrand
disease, type 1 = pA; cervical cancer metastasis = PB; tricho-dento-osseous syndrome = pB; systemic scleroderma = pb;
adenylate kinase deficiency, hemolytic anemia due to = Pb; vitisligo-associated multiple autoimmune disease susceptibility
1 = pC; adrenal carcinoma = PC; deficiency of adenylate kinase = Pc; hemorrhagic thrombocythemia = pc; androgen insensitivity
syndrome = PD; meningococcal infections = pD; thymoma = pd; hemophilia b = Pd; mammographic density = PE; xerosis = Pe;
plasmacytoma = pe; pregnancy complications, cardiovascular = pE; primary ciliary dyskinesia = Pf; esophagus adenocarcinoma =
pf; paranoid ideation = PF; pseudomonas infections = pF; mild dementia = PG; tendinopathy = pG; myelofibrosis = pg;
lyme disease = Pg; t-cell adult acute lymphocytic leukemia = ph; bile acid synthesis defect, congenital, 2 = PH; oral
infection = Ph; neuroticism = pH; progressive multifocal leukoencephalyopathy = pi; precancerous lesions = pI; motor
skills disorders = Pi; gonadoblastoma = PI; amyotrophyic lateral sclerosis 1 = PJ; uremic syndrome = pJ; long qt syndrome
11 = Pj; localized scleroderma = pj; arsenic poisoning = Pk; stress, psychological = pK; macular dystrophy, corneal type
1 = PK; burkitt lymphoma = pk; breast-ovarian cancer, familial, susceptibility to, 1 = PL; breast fibrocystic disease =
Pl; rubella = pl; superficial ulcer = pL; polycythemia vera = pm; childhood asthma = pM; megalanecephaly polymicrogyria-polydactyly
hydrocephalyus syndrome = PM; chronic arthritis = Pm; milroy's disease = PN; bowel dysfunction = Pn; hematocrit = pN;
nail-patella syndrome = pn; overweight and obesity = pO; adrenal gland disease = Po; eosinophilia = po; lymphedema, hereditary,
ii = PO; carcinogenicity = pP; b- and t-cell mixed leukemia = pp; alzheimer disease 5 = PP; hypospadias = Pp; juvenile
myelomaonocytic leukemia = pq; prostate cancer, hereditary, 7 = PQ; diabetic cataract = Pq; diminished ovarian reserve =
pQ; acute gastroenteritis = pR; campomelic dysplasia = pr; pten hamartoma tumor syndrome = PR; temporal arteritis =
Pr; dyschromatosis symmetrica hereditaria = ps; galactosemia = Ps; abo incompatibility = pS; cowden syndrome 6 = PS;
sensory peripheral neuropathy = Pt; azoospermia, nonobstructive = pt; cardioembolic stroke = pT; methamphetamine abuse =
PT; clinical infection = pU; prostate disease = pu; schimke immunoosseous dysplasia = Pu; progesterone resistance =
PU; stage, non-small cell lung cancer = PV; gastric cancer, intestinal = pV; thyroid hormone resistance syndrome = pv;
hypoprebetalipoproteinemia, acanthocytosis, retinitis pigmentosa, and pallidal degeneration = Pv; fast acetylator phenotype =
pW; beta thalassemia = pw; pantothenate kinase-associated neurodegeneration = Pw; psychiatric symptom = PW; neuroendocrine
tumor = pX; mitochondrial encephalyomyopathy = px; colon cancer stage iv = PX; endometriosis of ovary = Px; macroencephaly =
PY; multiple endocrine neoplasmia type 1 = pY; renal clear cell carcinoma = py; male pseudohermaphroditism due to deficiency
of testicular 17,20-desmolase = Py; dentinogenesis imperfecta - shield's type ii = Pz; open-angle glaucoma = pZ; acute
retention of urine = PZ; hemophagocytic lymphohistiocytosis = pz; large intestine cancer = q; alpha-2-macroglobulin deficiency =
Q; beta ketothiolase deficiency = qA; essential hypertension = qa; myxoid liposarcoma = QA; multiple symptoms = Qa;
corpus callosum agenisi neuronopathy = qB; anal squamous cell carcinoma = QB; leukemia, natural killer cell large granular
lymphocytic = Qb; sickle cell anemia = qb; atypical lipomatous tumor = QC; anastomosis, roux-en-y = Qc; chronic active
hepatitis = qC; obsessive-compulsive disorder = qc; beta thalassemia trait = qD; ovarian serous cystadenocarcinoma =
QD; fetal erythroblastosis = qd; schizophrenia 8 = Qd; alpha thalassemia = qe; chronic nk-cell lymphocytosis = QE; invasive
pulmonary aspergillosis = qE; intestinal benign neoplasm = Qe; multiple endocrine neoplasmia type 2a = QF; neonatal jaundice =
qf; aggressive cancer = qF; mesenchymal cell neoplasm = Qf; stomatitis = qg; multiple endocrine neoplasmia type 2b =
Qg; connective tissue benign neoplasm = QG; gingivitis = qG; atypical teratoid rhabdoid tumor = qH; factor viii deficiency =
qh; common wart = Qh; hypoinsulinemiac hypoglycemia with hemihypertrophy = QH; paranoid schizophrenia = qi; motor peripheral
neuropathy = qI; rett syndrome = Qi; insulin resistant diabetes = QI; vitisligo = qj; familial partial lipodystrophy =
QJ; follicular thyroid carcinoma = qJ; anal margin carcinoma = Qj; microcephaly = QK; von willebrand's disease = qk;
acoustic neuroma = Qk; salivary gland adenoid cystic carcinoma = qK; carotid artery thrombosis = QL; mixed oligodendroglioma-astrocytoma =
qL; hemolytic-uremic syndrome = ql; proteus syndrome = Ql; carcinosarcoma = Qm; pure red-cell aplasia = qm; morbid
obesity = qM; monster = QM; cervix carcinoma = qN; paraplegia = Qn; coccidioidomycosis = qn; syndactyly of toes with
fusion of bones = QN; glucosephosphate dehydrogenase deficiency = qo; hyperinsulinis = qO; cervix uteri carcinoma in
situ = QO; skin carcinoma = Qo; isobutyryl-coa dehydrogenase deficiency = qP; duodenal ulcer = qp; manganese poisoning =
QP; langerhans-cell histiocytosis = Qp; hemorrhagic disease = qq; childhood obesity = qQ; papillary adenocarcinoma =
Qq; lead poisoning, nervous system = QQ; embryonal rhabdomyosarcoma = Qr; pharyngitis = qr; lead poisoning, nervous
system, adult = QR; erythema nodosum = qR; anastomosis = qS; genitourinary cancer = QS; ovarian cystadenoma = Qs; hereditary
elliptocytosis = qs; kidney rhabdoid cancer = Qt; osmotic diarrhea = qT; tyrosinemia = QT; invasive ductal carcinoma =
qt; lung squamous cell carcinoma = Qu; social phobia = QU; intestinal infectious disease = qU; blood group incompatibility =
qu; porphyria = QV; bilateral breast cancer = qv; portal veins thrombosis = qV; papillary follicular thyroid adenocarcinoma =
Qv; erdheim-chester disease = Qw; uveitis = qW; sporadic breast cancer = qw; acute porphyria = QW; collecting duct
carcinoma = Qx; acute intermittent porphyri = QX; miller dieker syndrome = qx; fibromyalgia = qX; protoporphyria, erythropoietic,
x-linked dominant = QY; pyoderma gangrenosum = qY; grade iii astrocytoma = qy; large cell carcinoma = Qy; retinal cancer =
Qz; subacute sclerosisng panencephalyitis = qZ; juvenile pilocytic astrocytoma = qz; thalassemia intermedia = QZ; amyloidosis =
R; colorectal cancer = r; irritable bowel syndrome = ra; pyridoxine-responsive sideroblastic anemia = Ra; mixed germ
cell cancer = RA; laryngeal carcinoma = rA; mumps = RB; acyl-coa dehydrogenase family, member 9, deficiency of = rb;
tropical spastic paraparesis = rB; x chromosome-linked pyridoxine responsive sideroblastic anemia = Rb; pericardial effusion =
RC; prader-willi syndrome = rc; microcytic anemia = Rc; progeria = rC; achondroplasia = rD; mucopolysaccharidosis vi =
Rd; autonomic neuropathy = RD; preneoplasmtic state = rd; osteochondritis dissecans = rE; hypoproteinemia, hypercatabolic =
Re; 2-methylbutyrylglycinuria = re; hyperlipoproteinemia type iv = RE; aseptic meningitis = RF; hyperthyroxinemia,
familial dysalbuminemiac = Rf; dowling-degos disease = rF; cardiac arrest = rf; taqi polymorphism = rG; zinc, elevated
plasma = Rg; spondyloepimetaphyseal dysplasia, aggrecan type = rg; miller fisher syndrome = RG; ehlers-danlos syndrome,
type vii, autosomal dominant = Rh; spondyloepiphyseal dysplasia, congenita = rh; persistent infection = rH; kwashiorkor =
RH; duchenne and becker muscular dystrophy = Ri; dry eyes = rI; familial osteochondritis dissecans = ri; hemangioblastoma =
RI; mammary adenocarcinoma = rJ; hereditary angioedema = RJ; spondyloepiphyseal dysplasia, kimberley type = rj; isolated
somatotropin deficiency = Rj; occupational diseases = rk; hyperthyroxinemia = RK; smallpox = rK; dysferlinopathy = Rk;
amino acid metabolic disorder = rL; intervertebral disc displacement = rl; cholangiolocellular carcinoma = RL; pallidoluysian
degeneration = Rl; dry eye syndrome = rM; substance use disorder = Rm; croup = RM; sacroiliitis = rm; tendon injuries =
rn; congenital hypothyroidism = RN; leptospirosis = rN; danish type familial amyloid cardiomyopathy = Rn; disorder
of achilles tendon = ro; parvovirus b19 = Ro; gout, hprt-related = RO; teratoma = rO; de barsy syndrome = RP; lumbar
disc disease = rp; anti-diuresis = Rp; papillary renal cell carcinoma = rP; subcapsular cataract = RQ; hand osteoarthritis =
rq; chromophobe adenocarcinoma = rQ; single tumor = Rq; huntington's disease = rR; dysgenesis of corpus callosum =
RR; idiopathyic scoliosis = rr; acral pseudolymphomatous angiokeratoma of children (apache) = Rr; alcohol sensitivity,
acute = RS; intermediate coronary syndrome = rS; dysalbuminemiac hyperthyroxinemia = Rs; osteoarthritis of distal interphalangeal
joint = rs; tendinosis = rt; prostate cancer stage = RT; high-oxygen-affinity hemoglobin = Rt; cohen syndrome = rT; collagenopathy,
type 2 alpha 1 = ru; macroalbuminuric diabetic nephropathy = Ru; spinocerebellar ataxia-7 = rU; micropapillary carcinoma =
RU; fibromyxosarcoma = RV; acute viral hepatitis = Rv; osteochondrodysplasia = rv; brachydactyly = rV; fetal anemia =
Rw; hoyeraal hreidarsson syndrome = rW; epithelioid sarcoma = RW; arthropathy = rw; dysproteinemia = Rx; cystic disease =
rX; facioscapulohumeral muscular dystrophy = rx; digeorge syndrome = RX; multiple epiphyseal dysplasia = ry; neuronal
ceroid lipofuscinosis = rY; dental disorder = Ry; microphthalmia, isolated 8 = RY; choanal atresi = RZ; atrial heart
septal defect = rz; tooth disease = Rz; anisometropia = rZ; hemolytic anemia = S; adenoma = s; hangover, susceptibility
to (finding) = Sa; microvascular complications of diabetes, susceptibility to, 3 = sA; methylmalonate semialdehyde dehydrogenase
deficiency = SA; angle-closure glaucoma = sa; alcohol withdrawal delirium = Sb; 3-hydroxyisobutyric aciduria = SB; dyskeratosis
congenita = sb; heat stress disorders = sB; pyridoxine-dependent epilepsy = SC; familial melanoma = sc; water intoxication =
sC; melanosis = Sc; brain small vessel disease with hemorrhage = sd; emphysema = sD; secondary pulmonary hypertension =
SD; non-smoker = Sd; hangover = Se; lactic acidosis = SE; thrombophilia due to activated protein c resistance = se;
graft occlusion, vascular = sE; sarcopenia = sF; allanson pantzar mcleod syndrome = sf; glycogen storage disease xii =
SF; alcohol flush reaction = Sf; critical illness = sG; hereditary fructose intolerance syndrome = SG; hangover effect =
Sg; mass syndrome = sg; borna disease = SH; microvascular complications of diabetes, susceptibility to, 1 = sh; melanosis
coli = Sh; genetic diseases, inborn = sH; puerperal disorders = sI; congenital disorder of glycosylation type 1k = SI;
aortic valve disease 2 = si; alcohol problem = Si; long qt syndrome 2 = SJ; sarcoidosis, susceptibility to, 1 = sJ; albuminuria =
sj; drunk = Sj; multiple lacunar infarcts = Sk; segmental glomerulosclerosis = sk; coronary heart disease, susceptibility
to, 5 = sK; congenital disorder of glycosylation, type ip = SK; diabetic cardiomyopathyies = sl; congenital disorder
of glycosylatio = SL; chlorpropamide-alcohol flushing = Sl; angiotensin i-converting enzyme, benign serum increase =
sL; stomach disease = Sm; vascular system injuries = sm; homocysteinemia = sM; congenital disorder of glycosylation type
le = SM; congenital disorder of glycosylation type 1g = SN; cardiac fibrosis = sN; occupational dermatitis = Sn; abortion,
habitual = sn; hyperlipoproteinemia type ii = so; congenital disorder of glycosylation, type is = SO; mild cognitive
impairment = sO; ethylmalonic encephalyopathy = So; senior loken syndrome = Sp; congenital disorder of glycosylation
type ii = SP; bronchial hyperreactivity = sp; coronavirus infections = sP; congenital disorder of glycosylation type
1d = SQ; apraxia, oculomotor, cogan type = Sq; pregnancy complications, hematologic = sQ; activated protein c resistance =
sq; infant, premature, diseases = sr; altitude sickness = sR; hereditary macular dystrophy = Sr; congenital disorder
of glycosylation type 1h = SR; experimental organism basal cell carcinoma = sS; spastic diplegia = Ss; thrombophilia,
hereditary = ss; congenital disorder of glycosylation type 11 = SS; neuroblastoma, susceptibility to, 3 = ST; microvascular
angina = st; generalized glycogen storage disease of infants = sT; hyperprolinemia type 2 = St; kantaputra gorlin syndrome =
SU; aspirin resistance = sU; peptic ulcer hemorrhage = su; brain diseases, metabolic = Su; scarring = sV; ewing sarcoma/peripheral
primitive neuroectodermal tumor = SV; psychoses, substance-induced = sv; mental suffering = Sv; lymphatic metastasis =
sw; leukoaraiosis = sW; neural crest tumor = SW; convulsions/seizures = Sw; atrioventricular canal defect = sx; chronic
berylliosis = sX; neurological morbidity = Sx; disorder of pericardium = SX; ventricular remodeling = sy; phoria = Sy;
pleural disease = SY; infection of amniotic sac and membranes = sY; gastric lymphoma = SZ; status epilepticu = Sz; pulmonary
edema of mountaineers = sZ; sleep deprivation = sz; ovarian cancer = t; cancer = T; urticaria pigmentosa = Ta; unspecified
convalescence = tA; seasonal affective disorder = ta; bronchitis = TA; gangliocytoma = Tb; anoxemia = tB; panic disorder
1 = tb; generalized atherosclerosis = TB; chronic heart failure = tc; pigmented villanodular synovitis = TC; mastocytosis =
Tc; thrombophilia due to thrombin defect = tC; insulin sensitivity = td; ichthyosis, lamellar, 5 = TD; pulmonary plasma
cell granuloma = Td; primary iga nephropathy = tD; stroke, lacunar = te; ichthyosis, congenital, autosomal recessive
3 = TE; bladder adenocarcinoma = Te; brain injury, chronic = tE; convalescence = tF; spindle cell carcinoma = Tf; wolcott-rallison
syndrome = TF; aortic stiffness = tf; hypercortisolemia = tg; acute mountain sickness = tG; hypocalciuric hypercalcemia,
familial, type 1 = TG; ganglioneuroblastoma = Tg; pleomorphic lipoma = TH; endometrial stromal sarcoma = Th; reflux nephropathy =
th; nephroangiosclerosis = tH; pulmonary blastoma = Ti; schizophrenics = ti; nephrocalcinosis = TI; genetic hypertension =
tI; completed suicide = tJ; adenosquamous carcinoma = Tj; amyloid angiopathy = tj; hypophosphatasisa = TJ; pleomorphic
carcinoma = Tk; aspirin intolerance = tk; embryonal cancer = TK; proliferative retinopathy = tK; primary lateral sclerosis
juvenile = TL; malignant histiocytosis = Tl; cakut = tL; alexithymia = tl; liver inflammatory pseudotumor = Tm; amyotrophyic
lateral sclerosis 2, juvenile = TM; infant, very low birth weight = tM; age-associated memory impairment = tm; hereditary
spastic paralysis, infantile onset ascending = TN; chronic cough = tn; chronic closed-angle glaucoma = Tn; chronic urticaria =
tN; lone atrial fibrillation = tO; mixed anxiety and depressive disorder = To; beryllium disease = to; complicated
hereditary spastic paraplegia = TO; diabetic foot = tp; upper motor neuron disease = TP; bardet-biedl syndrome = Tp;
ablepharon macrostomia syndrome = tP; nodular glomerulosclerosis = tq; lateral sclerosis = TQ; alstrom syndrome = Tq;
aortic aneurysm of unspecified site without mention of rupture = tQ; frontonasal dysplasia = TR; lacunar infarction =
tr; congenital toxoplasmosis = Tr; fibrosis of pancreas = tR; ichthyosisform erythroderma, brocq congenital, nonbullous
form = Ts; macroangiopathy = ts; diabetic peripheral neuropathy = tS; frontonasal dysplasia 3 = TS; acrania = TT; self-healing
collodion baby = Tt; stalking = tT; diabetic microangiopathy = tt; dry cough = tU; asthma, aspirin-induced = Tu; midline
facial cleft - tessier cleft 0 = TU; clostridium = tu; post mi = tV; difficile (disorder) = tv; asthma, diminished response
to antileukotriene treatment in = Tv; parietal foramina 2 = TV; hyperhomocystinemia = Tw; end organ damage = tw; frontonasal
dysplasia 2 = TW; cardiovascular morbidity = tW; cognitive deterioration = tx; asthma, exercise-induced = Tx; coronary
artery atheroma = tX; craniosynostosis 5, susceptibility to = TX; cerebral white matter lesion = ty; moderate persistent
asthma = Ty; primary vesicoureteric reflux = tY; hereditary multiple exostoses = TY; parietal foramin = TZ; pulmonary
hypertension due to chronic exposure to high altitude = tZ; recurrent pregnancy loss = tz; aggressive periodontitis =
Tz; cholecystolithiasis = u; neurodegenerative disease = U; intersex conditions = UA; chronic small plaque psoriasis =
ua; bile acid synthesis defect, congenital, 4 = Ua; viral pneumonia = uA; microinvasive gastric cancer = uB; prostate
cancer, familial = Ub; pseudohermaphroditism = UB; persistent dry cough = ub; hypersensitivity vasculitis = UC; pre-eclampsia =
uC; skin fold (abnormality) = uc; oxyphilic adenoma = Uc; interstitial nephritis = uD; left ventricular dilatation =
ud; amelogenesis imperfecta = Ud; gonadal dysgenesis = UD; hypogonadism = UE; nephritis = uE; endocarditis = Ue; chronic
thromboembolic pulmonary hypertension = ue; ventricular dilatation = uf; klinefelter's syndrome = UF; central nervous
system cancer = Uf; pyelonephritis = uF; neurogenic bladder = Ug; portal hypertension = uG; anovulation = UG; left atrial
hypertrophy = ug; ovarian hyperstimulation syndrome = UH; malignant hypertension = uH; bacteriuria = Uh; silent myocardial
ischemia = uh; sertoli cell-only syndrome = UI; hydrocephalyus = uI; overanxious disorder = ui; renal infectious disease =
Ui; borderline personality disorder = uJ; cutis laxa, autosomal recessive, type i = Uj; persistent mullerian duct syndrome =
UJ; chronic systolic heart failure = uj; cri du chat = UK; antisocial personality disorder = uK; chronic uremia = uk;
geographic atrophy = Uk; sex differentiation disease = UL; hard drusen = Ul; low density lipoprotein receptor mutation =
ul; lipoid nephrosis = uL; alport syndrome, mental retardation, midface hypoplasia, and elliptocytosis = UM; alport
syndrome = uM; renal artery disease = um; kuhnt-junius degeneration = Um; asthma with copd = un; cutis lax = Un; meconium
aspiration syndrome = uN; neurogenic inflammation = UN; hydronephrosis = uO; amelogenesis imperfecta, hypoplastic/hypomaturation,
x-linked 1 = Uo; connective tissue disease = uo; extravasation of diagnostic and therapeutic materials = UO; megaloblastic
anemia = UP; heart valve disease = up; urinary tract obstruction = uP; osteopathyia striata cranial sclerosis = Up; sarcoidosis =
uQ; anterior ischemiac optic neuropathy = uq; japanese encephalyitis = Uq; spastic paraplegia 63, autosomal recessive =
UQ; pulmonary edema = uR; pontocerebellar hypoplasia = UR; migraine with aura = ur; female urogenital diseases = Ur;
chronic colitis = Us; mitral valve insufficiency = uS; intracranial aneurysm = us; erythrocyte amp deaminase deficiency =
US; musculoskeletal system disease = ut; diabetic neuropathy = uT; ovarian dysgenesis = Ut; glycogen storage disease
vi = UT; myopathy, centronuclear, autosomal recessive = UU; hypertensive heart disease = uU; streak gonad = Uu; migraine =
uu; hyperandrogenism = uV; anovulatory = Uv; renal hypertension = uv; autosomal recessive centronuclear myopathy =
UV; klinefelter's syndrome, male with more than two x chromosomes = Uw; autoimmune disease of the nervous system = UW;
nephrosclerosis = uW; raynaud disease = uw; anorchia = Ux; diabetic angiopathy = uX; congenital structural myopathy =
UX; peripheral vascular disease = ux; orphan diseases = UY; duchenne muscular dystrophy = uY; berylliosis = uy; inflammatory
polyarthritis = Uy; glycine encephalyopathy = UZ; hepatorenal syndrome = uZ; periodontitis = uz; chronic anterior uveitis =
Uz; obesity = v; multiple sclerosis = V; gnathomiasis = Va; stage i lung cancer = VA; nephrosis = va; uterine fibroid =
vA; von hippel-lindau disease = VB; pulmonary sarcoidosis = vB; coronary thrombosis = vb; prothrombin g20210a mutation =
Vb; amyotrophyic lateral sclerosis 9 = Vc; craniometaphyseal dysplasia, autosomal dominant = VC; hypertrophyic cardiomyopathy =
vc; retinal vascular occlusion = vC; optic nerve disease = vd; exfoliation syndrome = vD; amyotrophyic lateral sclerosis,
sporadic = Vd; chondrocalcinosis 2 = VD; wegener's granulomatosis = ve; malaria, susceptibility to = VE; complex regional
pain syndrome = vE; acute-phase reaction = Ve; human anaplasmosis due to anaplasma phagocytophilum = VF; cushing's syndrome =
vf; gout acute = Vf; aortic disease = vF; familial lipoprotein lipase deficiency = vG; adrenal gland hyperfunction =
vg; obstructive chronic pancreatitis = Vg; schwartz-lelek syndrome = VG; inherited blood coagulation disease = vh;
proctitis = Vh; azoospermia = vH; familial chondrocalcinosis = VH; hepatitis = vi; alveolar soft part sarcoma = Vi; calcium
pyrophosphate dihydrate deposition = VI; marfan syndrome = vI; pterygium = Vj; secondary hyperparathyroidism = vj; turner
syndrome = vJ; hereditary spherocytosis = VJ; diastrophyic dysplasia = vK; ankylosis = VK; anaphylaxis = Vk; retinal
detachment = vk; major depressive disorder = vL; capillary disease = vl; cardiac arrhythmia, ankyrin-b-related = VL;
astrocytoma, low grade = Vl; pad mass = Vm; angioedema = vM; nephronophthisis = vm; intestinal volvulu = VM; mental retardation,
autosomal recessive 37 = VN; normal pressure hydrocephalyus = vN; femoral artery occlusion = Vn; cerebral atherosclerosis =
vn; neuroma = VO; migraine without aura = vo; rheumatic fever = vO; pulmonary arterial hypertension associated with
portal hypertension = Vo; renovascular hypertension = vP; goiter = Vp; cyclothymic disorder = VP; endomyocardial fibrosis =
vp; extramedullary plasmacytoma = Vq; alcoholic cardiomyopathy = vq; chondrocalcinosis = VQ; aortic valve stenosis =
vQ; superficial urinary bladder cancer = Vr; carotid stenosis = vr; retinal vein occlusion = vR; opioid-related disorders =
VR; reflex sympathyetic dystrophy = vS; dopamine receptor d2, reduced brain density of = VS; transient cerebral ischemia =
vs; ectopic pregnancy = Vs; aneurysm of ascending aorta = Vt; germ cell cancer = vT; retinopathy of prematurity = vt;
language disorder = VT; neuroleptic malignant syndrome = VU; intracranial arterial disease = vu; gaucher's disease =
vU; telangiectatic focal nodular hyperplasia = Vu; lymphedema = Vv; takayasu's arteritis = vV; retinal artery occlusion =
vv; cocaine dependencye = VV; familial nephrotic syndrome = vW; scimitar syndrome = VW; focal segmental glomerulosclerosis =
vw; capillary leak syndrome = Vw; proliferative diabetic retinopathy = vx; glycogen storage disease v = vX; vasculogenic
impotence = Vx; child development disorders, pervasive = VX; angiosarcoma = Vy; kbg syndrome = VY; diabetic retinopathy =
vy; glycogen storage disease ii = vY; contact dermatitis = vZ; caroli disease = Vz; temporal lobe neoplasm = VZ; background
diabetic retinopathy = vz; fibrodysplasia ossificans progressiva = w; age related macular degeneration = W; bronchiolitis
obliterans = wa; renal oncocytoma = WA; thrombocytopenia chromosome breakage = Wa; vascular dementia = wA; parapsoriasis =
WB; polycystic kidney disease = wB; acute pancreatitis = wb; gallbladder disease = Wb; hepatopulmonary syndrome = wC;
methylmalonic aciduria cblb type = WC; nephronophthisis 16 = Wc; severe acute respiratory syndrome = wc; psoriatic arthritis =
wD; osteogenesis imperfecta, levin type = Wd; hemangioma, capillary infantile = WD; renal artery obstruction = wd;
hyperhomocysteinemia = wE; seckel syndrome 1 = WE; embryonic cyst = We; anuria = we; ocular hypertension = wF; mental
retardation-hypotonic facies syndrome, x-linked, 1 = WF; familial mediterranean fever = wf; diabetic gastroparesis =
Wf; brain infarction = wg; growth retardation, alopecia, pseudoanodontia and optic atrophy = WG; spinocerebellar ataxia,
autosomal recessive 10 = Wg; vesicoureteral reflux = wG; sarcoma 180 = Wh; systolic heart failure = wH; cellulitis =
wh; alpha thalassemia-mental retardation syndrome = WH; myoclonic dystonia = Wi; middle cerebral artery infarction =
wi; severe intellectual disabilityies = WI; diastolic heart failure = wI; dystonia 24 = Wj; intermittent claudication =
wj; mitral valve prolapse = wJ; inhalation anthrax = WJ; arrhythmogenic right ventricular cardiomyopathy = wK; breast
medullary carcinoma = WK; congenital diaphragmatic hernia = wk; primary dystonia = Wk; tricuspid valve insufficiency =
wl; nijmegen breakage syndrome = WL; cervical dystonia = Wl; atrial fibrillation = wL; epstein-barr virus hepatitis =
WM; seminoma = wm; obstructive sleep apnea = wM; generalized dystonia = Wm; cardiac arres = wN; persian gulf syndrome =
wn; gingival hypertrophy = WN; bethlem myopathy = Wn; uremia = wo; miyoshi muscular dystrophy 3 = Wo; lissencephaly,
x-linked, 1 = WO; middle cerebral artery occlusion = wO; muscular dystrophy, limb-girdle, type 21 = Wp; dysgerminoma
of ovary = wp; paratuberculosis = WP; psychological symptom = wP; temporal pain = wQ; asymptomatic diseases = WQ; acute
pyelonephritis = wq; fukuyama muscular dystrophy = Wq; disorder of skeletal system = Wr; bladder pain syndrome = WR;
fungal infectious disease = wR; werner syndrome = wr; acute peritonitis = WS; distal muscular dystrophy = Ws; hartnup
disease = wS; aortic valve insufficiency = ws; proteinuria = wt; peniale cancer = WT; common variable immunodeficiency =
Wt; friedreich ataxia = wT; penias carcinoma = WU; testis seminoma = wu; aortic valve disease = wU; seborrheic dermatitis =
Wu; penias squamous cell carcinoma = WV; sucrase-isomaltase deficiency, congenital = Wv; myocarditis = wV; agoraphobia =
wv; panic disorder = ww; epidemic diarrhea = Ww; thanatophoric dysplasia, type i = wW; gastric cardia adenocarcinoma =
WW; bare lymphocyte syndrome 2 = WX; endplate acetylcholinesterase deficiency = wX; pulmonary embolism = wx; chromosome
8, monosomy &p = Wx; chronic fatigue syndrome = wy; isaacs syndrome = wY; frontotemporal lobar degeneration = WY; leukostasis =
Wy; endometriosis of uterus = WZ; organophosphate poisoning = wZ; perinatal necrotizing enterocolitis = wz; brucella
melitensis brucellosis = Wz; acute lymphocytic leukemia = x; colonic benign neoplasm = X; congenital myasthenic syndrome =
xA; mhc class ii deficiency = Xa; congenital pigmentation = XA; amphetamine-related disorders = xa; growth disorders =
xb; frontotemporal dementia = xB; urethritis = Xb; hemorrhagic fever with renal syndrome = XB; lambert-eaton myasthenic
syndrome = xC; chediak-higashi syndrome = XC; ovarian clear cell adenocarcinoma = Xc; alzheimer disease type 2 = xc;
prenatal injuries = xd; gonorrhe = Xd; herpesviridae infections = xD; bartter disease = XD; cyclic hematopoiesis = XE;
myeloproliferative disorder, chronic, with eosinophilia = Xe; myasthenia gravis, autoimmune, experimental = xe; collagen
arthritis = xE; stress disorders, traumatic = xf; hereditary sensory-motor neuropathy, type i = xF; pregnancy loss, recurrent,
susceptibility to, 3 = Xf; albinism with hemorrhagic diathesis and pigmented reticuloendothelial cells = XF; treacher
collins syndrome = Xg; hemolytic disorder = xG; epidermaolysis bullosa with pyloric atresia = XG; cognitive deficits =
xg; acute stress = xh; varicella zoster = xH; spastic paraplegia 47, autosomal recessive = XH; angiomyolipoma = Xh;
cerebellar function = XI; absence of sensation = xi; lymphangioleiomyomatosis = Xi; pseudorabies = xI; plasmodium vivax
infection = xJ; congenital myasthenia = xj; inherited epidermaolysis bullosa = XJ; subependymal giant cell astrocytoma =
Xj; plasmodium vivax malaria = xK; epidermal differentiation complex = Xk; migraineous vertigo = XK; fatigability =
xk; rh isoimmunization = xL; food allergy = Xl; slow channel syndrome = xl; propionic acidemia = XL; myasthenic syndrome =
xm; epidermaolysis bullosa = XM; genital herpes = xM; eosinophili = Xm; junctional epidermaolysis bullosa = XN; ileal
diseases = xN; xanthinuri = Xn; familial olivopontocerebellar atrophy = xn; erythrokeratodermaia variabilis 3 = Xo; anemia
of chronic disease = xO; anxiety neurosis = xo; bullous pemphigoid = XO; disorder of copper metabolism = Xp; irritant
dermatitis = xP; anxiety state = xp; spastic paraplegia 51, autosomal recessive = XP; serous cystadenocarcinoma = xQ;
mental retardation, x-linked, syndromeic 5 = Xq; cerebral arteriosclerosis = xq; spastic paraplegia-50, autosomal recessive =
XQ; dandy-walker syndrome = Xr; mycosis fungoides = xR; parkinsonism, experimental = xr; spastic paraplegia 52, autosomal
recessive = XR; stevens-johnson syndrome = xS; muscular dystrophy = xs; spastic paraplegia 48, autosomal recessive =
XS; pustular psoriasis = Xs; branchio oculo facial syndrome = Xt; learning disability = xt; granular corneal dystrophy
type i = xT; nevi and melanomas = XT; acrodystrophyic neuropathy = xU; alveolar rhabdomyosarcoma = Xu; plant diseases =
XU; blood platelet disease = xu; total hypotrichosis, mari type = xV; nodular malignant melanoma = XV; orbital disease =
xv; familial benign hypercalcemia, type 3 = Xv; hypercalcemi = Xw; clubfoot = XW; movement disease = xw; pseudohypoparathyroidism,
type ib = xW; thanatophoric dysplasia = xx; bladder transitional cell carcinoma = XX; hermansky pudlak syndrome 2 = Xx;
autosomal dominant retinitis pigmentosa = xX; bitter taste = xY; nicotine dependency = XY; thyroiditis = xy; bartter
syndrome, type 3 = Xy; inflammatory pain = xZ; usher syndrome, type 1b = Xz; sleeping sickness = XZ; centronuclear myopathy =
xz; rheumatoid arthritis = y; melanoma = Y; dyslexia = yA; mosaic variegated aneuploidy syndrome = Ya; neuroepithelioma =
ya; rectal disease = YA; desmoid disease, hereditary = Yb; duodenum cancer = YB; rhizomelic chondrodysplasia punctata,
type 1 = yB; pulmonary cystic fibrosis = yb; taste sweet = yc; deficiency of oxidase = yC; intestinal polyps = Yc; duodenum
adenocarcinoma = YC; peroxisomal acyl-coa oxidase deficiency = yD; seborrheic keratosis = YD; acquired aplastic anemia =
yd; intestinal polyposis = Yd; ectrodactyly, ectodermal dysplasia, and cleft lip/palate syndrome 1 = Ye; parathyroid
carcinoma = YE; osteoporosis, postmenopausal = yE; renal sclerosis with hypertension = ye; rectum adenocarcinoma = YF;
desmoid tumor caused by somatic mutation = Yf; autoimmune thyroid disease = yF; clumsiness - motor delay = yf; autoimmune
diabetes = yG; protein s deficiency = YG; bone cancer = yg; brain tumor-polyposis syndrome 2 (disorder) = Yg; congenital
plasminogen deficiency = Yh; parathyroid gland disease = yh; nevoid basal cell carcinoma syndrome = YH; immunopathyology =
yH; carcinoid tumor = Yi; acinar cell carcinoma = YI; cryptorchidism = yi; generalized vitisligo = yI; jejunal neoplasm =
YJ; nephrogenic diabetes insipidus = yj; convulsive disorder = yJ; traditional serrated adenoma = Yj; aniridia type
1 = yK; hyperprothrombinemia = Yk; anal carcinoma = YK; skin cancer = yk; acrodysostosis = yl; signet ring cell adenocarcinoma =
YL; congenital hypertrophy = Yl; gilles de la tourette syndrome = yL; vascular cancer = ym; palindromic rheumatism =
yM; bacteroides fragilis = Ym; extrahepatic bile duct carcinoma = YM; glucose-6-phosphate dehydrogenase deficiency =
yN; duodenal disease = YN; kallmann syndrome = yn; adenoviral infections = Yn; gastric adenoma = Yo; leopard syndrome =
yO; pseudomyxoma peritonei = YO; pituitary adenoma = yo; congenital hypertrophy of retinal pigment epithelium = Yp;
embryoma = yp; generalized anxiety disorder = yP; myoepithelial carcinoma = YP; branchiootorenal syndrome = yQ; small
intestine adenocarcinoma = YQ; kaposi's sarcoma = yq; polyposis of gastric fundus without polyposis coli = Yq; early
myoclonic encephalyopathy = yr; systemic mastocytosis = yR; hepatocellular fibrolamellar carcinoma = YR; intestinal adenocarcinoma =
Yr; noonan syndrome = yS; juvenile polyposis coli = Ys; pilomatrixoma = YS; cerebral cortex myoclonus = ys; neoplasm
invasiveness = yt; lafora disease = yT; basaloid squamous cell carcinoma = YT; blood in stool = Yt; progressive myoclonus
epilepsy = yU; mixed-type liposarcoma = YU; clinical sepsis = Yu; hereditary hemochromatosis = yu; eccrine porocarcinoma =
YV; pregnancy in diabetics = yv; mesangial proliferative glomerulonephritis = yV; poroma = Yv; iron deficiency = yw;
polyp of duodenum = Yw; intussusception = YW; chronic inflammatory demyelinating polyradiculoneuropathy = yW; epilepsy
with generalized tonic-clonic seizures = yX; toxoplasmosis = yx; mucous membrane hyperplasia = Yx; hypotrichosis simplex =
YX; ileal pouch = Yy; hemochromatosis = yy; lymphoproliferative syndrome, x-linked, 2 = YY; restless legs syndrome =
yY; dentatorubral-pallidoluysian atrophy = yZ; primary systemic amyloidosis = YZ; infantile cerebellar-retinal degeneration =
yz; duodenal benign neoplasm = Yz; mental depression = Z; allergic asthma = z; gomez lopez hernandez syndrome = za;
steatocystoma multiplex = ZA; candidiasis, cutaneous = Za; primary immune deficiency disorder = zA; hyper-igm immunodeficiency
syndrome, type 1 = zb; infection by candida albicans = Zb; hyperalphalipoproteinemia 1 = ZB; hyperalgesia, thermal =
zB; localized amyloidosis = Zc; macular degeneration, age-related, 1 = ZC; combined immunodeficiency with autoimmunity
and spondylometaphyseal dysplasia = zc; renal ischaemia = zC; mechanical allodynia = zD; hepatic lipase deficiency =
ZD; dysgammaglobulinemia = Zd; spondyloenchondrodysplasia = zd; asthenozoospermia = ZE; fowlpox = ze; breast disease =
zE; agammaglobulinemia = Ze; oral candidiasis = Zf; breast cyst = zF; hepatorenal form of glycogen storage disease =
ZF; oestrogen deficiency = zf; hypobetalipoproteinemia, familial = ZG; polydactyly = zG; callosities = zg; infectious
mononucleosis = Zg; corneal pannus = zh; lenticular opacities = Zh; hydatidiform mole, recurrent, 1 = ZH; prostatitis =
zH; hypobetalipoproteinemia, familial, apolipoprotein b = ZI; adenocarcinoma in situ = zI; reaction to colouring =
zi; micronuclei, chromosome-defective = Zi; monoclonal gammopathy = zJ; tophus = zj; papillomavirus infections = Zj;
hypobetalipoproteinemia, familial, 1 = ZJ; primary cutaneous follicle center lymphoma = zK; cyanotic congenital heart
disease = zk; hypobetalipoproteinemia, familial, 2 = ZK; subcutaneous fibrosis = Zk; pancreatic disorders (not diabetes) =
zl; hodgkin's-like = zL; beta thalassemia = ZL; pneumoniatis = Zl; distemper = zm; lown-ganong-levine syndrome = zM;
acanthocytosis = ZM; keratosis = Zm; hairy cell leukemia = zn; meningitis = Zn; malonic aciduria = zN; alcoholic steatohepatitis =
ZN; otitis media = zo; transient global amnesia = Zo; ruptured cerebral aneurysm = ZO; combined malonic and methylmalonic
aciduria = zO; mammary neoplasms, experimental = zP; fat intolerance = ZP; osteogenesis imperfecta = zp; smooth muscle
tumor = Zp; mammary neoplasms, animal = zQ; renal osteodystrophy = zq; intraocular retinoblastoma = Zq; lentivirus infections =
ZQ; synovitis = zr; pediatric intraocular retinoblastoma = Zr; triple vessel disease = ZR; starvation = zR; osteopetrosis =
zs; adult growth hormone deficiency = ZS; symptoms in children = zS; pulmonary venoocclusive disease = Zs; bulimia
nervosa = ZT; poliomayelitis = zT; fish-eye disease = Zt; bone giant cell tumor = zt; abetalipoproteinemia = ZU; cholesteryl
ester transfer protein deficiency = Zu; syndactyly = zU; giant cell tumor = zu; hypobetalipoproteinemia = ZV; combined
hyperlipidaemia = Zv; reticulosarcoma = zv; craniosynostosis = zV; ptosi = zW; holoprosencephaly = ZW; bone disease =
zw; mixed hyperlipidemia = Zw; familial hyperchylomicronemia syndrome = Zx; adenocarcinoma, endometrioid = zX; acrodermatitis
enteropathyica = zx; ariboflavinosis = ZX; hyperlipoproteinemia type v = Zy; mycoplasma infections = zy; postmenopausal
endometrium = zY; scaly skin = ZY; enzymopathy = zz; hyperlipoproteinemia type iii = Zz; tubular adenocarcinoma = ZZ;
premature menopause = zZ;

1-60. (canceled)

61. A method for treating a subject in need thereof by increasing the level of methylmalonyl-CoA mutase (MUT), the method comprising administering to the subject a pharmaceutical composition comprising (a) at least one high performance liquid chromatographically-purified mRNA polynucleotide encoding MUT, said mRNA comprising a sequence at least 95% identical to SEQ ID NO: 5601; and (b) a pharmaceutically acceptable lipid nanoparticle.

62. The method according to claim 61, wherein the pharmaceutical composition comprises a lipid which is selected from the group consisting of DLin-DMA, DLin-K-DMA, DLin-KC2-DMA, 98N12-5, C12-200, DLin-MC3-DMA, 1,2-dimyristoyl-sn-glycero-3-phosphoethanol-amine-N-methoxy(polyethyleneglycol) (PEG-DMG) and PEGylated lipids and mixtures thereof.

63. The method according to claim 61, wherein the pharmaceutical composition is administered by injection.

64. The method according to claim 61, wherein the pharmaceutical composition is administered at a total daily dose of between 0,001 μg and 150 μg.

65. The method according to claim 61, wherein the at least one mRNA polynucleotide comprises at least one modification.

66. The method according to claim 65, wherein the at least one modification is a modification selected from the group consisting of codon optimization, untranslated region (UTR) modification, addition of a poly(A) tail, addition of a 5′-CAP structure, and incorporation of at least one chemically modified nucleotide.

67. The method according to claim 66, wherein the at least one modification is the addition of a 5′-CAP structure selected from the group consisting of Cap1, anti-reverse CAP analogue (ARCA), inosine, N1-methyl-guanosine, 2′-fluoro-guanosine, 7-deaza-guanosine, 8-oxo-guanosine, 2-amino-guanosine, locked nucleic acid (LNA)-guanosine, and 2-azido-guanosine.

68. The method according to claim 66, wherein the at least one modification is the addition of a 5′-CAP structure that is a chemically modified ARCA-CAP.

69. The method according to claim 68, wherein the ARCA-CAP is modified with phosphorothioate.

70. The method according to claim 66, wherein the chemically modified nucleotide is selected from the group consisting of pyridine-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl)adenosine, N6-glycinylcarbamoyladenosine, N6-threonylcarbamoyladenosine, 2-methylthio-N6-threonylcarbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

71. The method according to claim 63, wherein the pharmaceutical composition is administered by intravenous injection.

72. The method of claim 61, wherein mRNA comprises a sequence at least 97% identical to SEQ ID NO: 5601.

73. The method of claim 61, wherein the subject has methylmalonic acidemia (MMA).

74. The method of claim 66, wherein the chemically modified nucleotide is 5-methoxyuridine.

75. The method of claim 65, wherein the mRNA comprises a 5′Cap, a 5′ UTR, a 3′ UTR and a Poly-A sequence.

76. The method of claim 75, wherein the coding sequence of the mRNA is codon optimized.

77. The method of claim 61, wherein the subject has a cancer.

78. The method of claim 77, wherein the subject has breast cancer, colorectal cancer, thyroid cancer; prostate cancer, cervical cancer, meningioma, non-small cell lung carcinoma or retinoblastoma.

79. The method of claim 4, wherein the pharmaceutical composition comprises PEG-DMG.
